(19) United States
(12) Patent Application Publication
(10) Pub. No.: US 2024/0194304 A1
(43) Pub. Date: Jun. 13, 2024

(54) Prediction Method, Prediction Device, and Prediction Program for New Indication of Desired Known Drug or Equivalent Material Thereof

(71) Applicant: KARYDO THERAPEUTIX, INC., Tokyo (JP)

(72) Inventor: Narutoku Sato, Soraku-gun, Kyoto (JP)

(73) Assignee: KARYDO THERAPEUTIX, INC., Tokyo (JP)

(21) Appl. No.: 17/793,468

(22) PCT Filed: Jan. 15, 2021
(86) PCT No.: PCT/JP2021/001277

§ 371 Date: Jul. 18, 2022

(30) Foreign Application Priority Data
Jan. 17, 2020 (JP) 2020-006259

(51) Int. Cl. G16C 20/70; G16H 20/10

(52) U.S. Cl. CPC G16C 20/70; G16H 20/10

(57) ABSTRACT

An object of the present invention is to achieve drug repositioning and/or drug repurposing without conducting animal experiments.

The problems are solved by a method for predicting a new indication for a known drug of interest or its equivalent substance, including the step of predicting a new indication for the known drug of interest or its equivalent substance using an artificial intelligence model trained based on test data which is information about adverse events and/or side effects reported for the known drug of interest or its equivalent substance.

### TECHNICAL FIELD

This description discloses a method, a device and a program for predicting a new indication for a known drug of interest or its equivalent substance; a method, a device and a program for training an artificial intelligence for making the prediction; and a composition for use against a predicted new indication.

### BACKGROUND ART

Discovery and development of a drug takes a long time and a huge amount of money, and there are risks involved in the process. It is said that discovery and development of a new drug takes an average of 12 years and costs about 2.6 billion dollars. Despite such tremendous efforts, it is said that only 13.8% of drug candidates succeed in clinical trials. To avoid these problems, several strategies and approaches have been proposed and put into practice. One of them is repositioning and repurposing (DR) of existing drugs (Non-Patent Document 1).

DR is a method of exploring further therapeutic indication(s) (TI(s)) for clinically approved existing pharmaceutical products. In DR, the required development time is short and the cost is not as high as that for new drug development. Also, the pharmaceutical products have already been approved for use in treating at least one disease or symptom in humans. Thus, there is less concern about toxicity in humans. It is, therefore, possible in DR to skip the phase I clinical trials and proceed immediately to the phase II trials. In addition, because these drugs are already mass-produced for human use, the production process for clinical use has already been optimized. These characteristics of DR can lead to significant saving of time and cost in the development and approval processes (Non-Patent Document 1).

Currently, there are two main types of DR approaches. One of them is a method in which new indications and/or applications for each DR drug candidate are rationally designed and screened by thoroughly studying and understanding its biological, pharmacological, and/or structural properties. The other is a method depending on serendipity (incidental discovery). In other words, there may be the case where new indication and/or new applications are discovered incidentally during preclinical trials, clinical trials, and/or monitoring of new drugs in the real world. These general approaches are relatively ineffective and are the bottleneck of the current DR discovery process (Non-Patent Document 1).

As a method for assisting the exploration of candidate substances for new drugs in the development of a new drug, Patent Document 1 discloses a method including comparing test data of an organ-related index factor in each organ obtained from cells or tissues derived from one or more organs of individuals to which a test substance has been administered with preliminarily determined corresponding standard data of the organ-related index factor to obtain a pattern similarity indicating the similarity of the pattern of the organ-related index factor, and predicting the efficacies or side effects of the test substance in the one or more organs and/or in organs other than the one or more organs using the pattern similarity of the organ-related index factor as an index.

Also, as a method for predicting efficacies or side effects of a candidate substance in the development of a new drug, Patent Document 2 and non-Patent Document 3 disclose an artificial intelligence model for predicting one or more effects of a test substance on humans from the behavior of transcriptome in multiple different organs which are the same as those collected from non-human animals to which the test substance has been administered to prepare training data.

The method includes inputting a data collection indicating the behavior of transcriptome in multiple different organs collected from non-human animals to which multiple known substances with known effects on humans have been individually administered for each of the non-human animals and data indicating known effects of each known substance on humans into the artificial intelligence model as training data to train the artificial intelligence model.

### CITATION LIST

### Patent Document

[Patent Document 1] WO2016/208776
[Patent Document 2] Japanese Patent No. 6559850

### Non-Patent Document

[Non-Patent Document 1] Pushpakom, S et al., (2019): Nature reviews Drug discovery 18, 41-58.
[Non-Patent Document 2] Perwitasari, O et al., (2013): Pharmaceuticals (Basel) 6, 124-160.
[Non-Patent Document 3] Kozawa, S et al., (2020): iScience (DOI: 10.1016/j. isci.2019.100791)
[Non-Patent Document 4] Li, J., and Lu, Z. (2012): Proceedings (IEEE Int Conf Bioinformatics Biomed) 2012, 1-4.

### SUMMARY OF INVENTION

### Technical Problem

On the other hand, in the methods described in Patent Document 1, Patent Document 2 and Non-Patent Document 3, it is necessary to administer drugs to mice and collect one or more organs from the mice to prepare data indicating the behavior of a biomarker in respective organs.

Also, for more accurate prediction, it is necessary to administer many types of drugs to mice individually to acquire data indicating the behavior of a biomarker in one or more organs. Thus, a reasonable number of animal experiments are required.

One object of the present invention is to achieve drug repositioning and/or drug repurposing without conducting animal experiments.

### Solution to Problem

As a result of intensive studies, the present inventor found that it is possible to achieve drug repositioning and/or drug repurposing using an artificial intelligence based on information about adverse events and/or side effects related to various known drugs and information about indications therefor registered in a public drug database or the like.

The present invention has been made based on the finding, and includes the following aspects.

Embodiment 1. A method for predicting a new indication for a known drug of interest or its equivalent substance, including: a step of predicting a new indication for the known drug of interest or its equivalent substance using an artificial intelligence model trained based on test data which is information about adverse events and/or side effects reported for the known drug of interest or its equivalent substance.

Embodiment 2. The prediction method according to Embodiment 1, in which the information about adverse events and/or side effects corresponds to the presence or absence of multiple adverse events and/or side effects, or occurrence frequencies thereof.

Embodiment 3. The prediction method according to Embodiment 1 or 2, in which the artificial intelligence model corresponds to one indication.

Embodiment 4. The prediction method according to Embodiment 1 or 2, in which the artificial intelligence model corresponds to multiple indications.

Embodiment 5. A device for predicting a new indication for a known drug of interest or its equivalent substance, including a processing part, in which the processing part is configured to predict anew indication for the known drug of interest or its equivalent substance using an artificial intelligence model trained based on test data which is information about adverse events and/or side effects reported for the known drug of interest or its equivalent substance.

Embodiment 6. A computer program for predicting a new indication for a known drug of interest or its equivalent substance, executable by a computer to cause the computer to execute processing including a step of predicting a new indication for the known drug of interest or its equivalent substance using an artificial intelligence model trained based on test data which is information about adverse events and/or side effects reported for the known drug of interest or its equivalent substance.

Embodiment 7. A method for training an artificial intelligence model, including training an artificial intelligence model by means of a set of training data, in which each item of training data is data in which (I) information about adverse events and/or side effects reported for individual known drugs is/are associated with (II) indication data reported for the known drugs, and in which the artificial intelligence model predicts a new indication for a known drug of interest or its equivalent substance.

Embodiment 8. The training method according to Embodiment 7, in which each item of the training data is generated by linking a label indicating an indication for the known drug and information about adverse events and/or side effects reported for the known drug by means of a label indicating the name of the known drug.

Embodiment 9. The training method according to Embodiment 7 or 8, in which the information about adverse event and/or side effects corresponds to the presence or absence of multiple adverse events and/or side effects; or occurrence frequencies of adverse events and/or side effects.

Embodiment 10. The training method according to any one of Embodiments 7 to 9, in which the artificial intelligence model corresponds to one indication.

Embodiment 11. The training method according to any one of Embodiments 7 to 9, in which the artificial intelligence model corresponds to multiple indications.

Embodiment 12. A device for training an artificial intelligence model, comprising a processing part, in which the processing part is configured to train an artificial intelligence model by means of a set of training data, in which each item of training data is data in which (I) information about an adverse event and/or side effect reported for an individual known drug is associated with (II) indication data reported for the known drug, and in which the artificial intelligence model predicts a new indication for a known drug of interest or its equivalent substance.

Embodiment 13. A program for training an artificial intelligence model, executable by a computer to cause the computer to execute processing including a step of training an artificial intelligence model by means of a set of training data, in which each item of training data is data in which information about an adverse event and/or side effect reported for an individual known drug is associated with indication data reported for the known drug, and in which the artificial intelligence model predicts a new indication for a known drug of interest or its equivalent substance.

Embodiment 14. A composition containing a drug selected from a drug list shown in the description in order to use the drug in treatment or prevention of a new indication predicted for the drug by the prediction method according to Embodiment 1 or 2.

### Advantageous Effect of Invention

It is possible to achieve drug repositioning and/or drug repurposing without conducting animal experiments.

### BRIEF DESCRIPTION OF DRAWINGS

FIG. 1 illustrates an overview of drug repositioning and/or drug repurposing disclosed in this description.

FIG. 2 shows examples of training data. FIG. 2(A) shows an example of a set of training data for nerve injury. FIG. 2(B) shows a set of training data for type 2 diabetes mellitus.

FIG. 3 illustrates a hardware configuration of a training device 10.

FIG. 4 shows a flowchart of training processing.

FIG. 5 illustrates a hardware configuration of a prediction device 20.

FIG. 6 shows a flowchart of prediction processing.

FIG. 7 shows distributions of accuracy, recall and precision scores for all drugs.

FIG. 8 shows respective scores of the top 50 drugs having accuracy, precision and recall scores that are all 1.0 among drugs for which indication prediction was performed.

FIG. 9 shows distribution of accuracy, recall and precision scores for all indications.

FIG. 10 shows respective scores of the top 50 indications having accuracy, precision and recall scores that are all 1.0 among predicted indications.

FIG. 11 shows results of blind evaluation.

FIG. 12 shows comparison between the invention of this disclosure and a prior art.

### DESCRIPTION OF EMBODIMENTS

1. Overview of training method and prediction method, and description of terms First, a method for training an artificial intelligence and a prediction method as certain embodiments of this disclosure are outlined. The prediction method predicts a new indication for a known drug of interest or its equivalent substance (in this description, a known drug or its equivalent substance may be collectively referred to simply as “known drug or the like”). Preferably, the prediction method predicts an indication (therapeutic indication: TI) for a known drug or the like based on information about adverse events (AEs) and/or side effects (SEs) reported for the known drug of interest or its equivalent substance. The prediction is achieved using artificial intelligence models. Here, for convenience sake, an example using adverse events is shown.

### (1) Training Phase

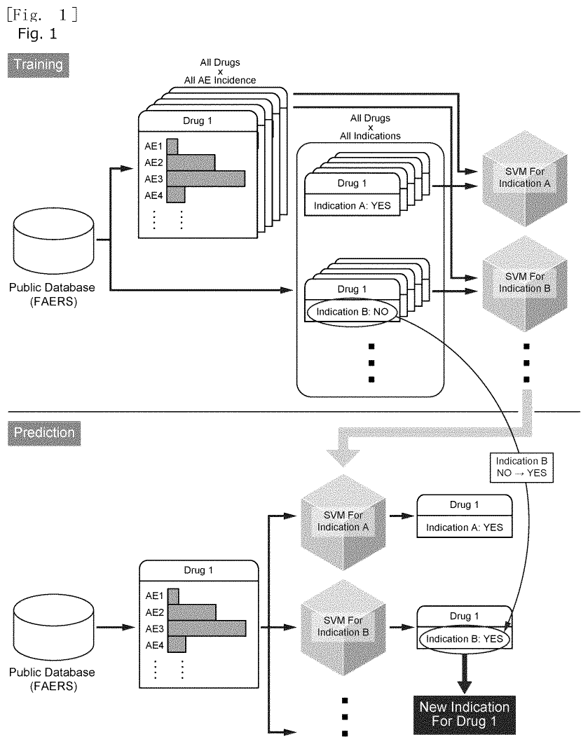

The upper part of FIG. 1 shows an overview of a training phase. Training data includes information about adverse events reported for known drugs and indication data reported for the known drugs based on information available from a public drug database. FAERS, which is described later, is shown as an example in FIG. 1, and reported and unreported adverse events are registered for each drug in this drug database. In other words, information about whether or not each of multiple adverse events has appeared is registered for each drug. In this description, information about whether or not a certain adverse event has appeared (the presence or absence of a certain adverse event) for one drug is referred to as adverse event data. Adverse event data is linked with a label indicating a drug name that indicates to which drug the adverse event data belongs. In the drug database, multiple items of adverse event data are registered per drug, and these constitute a set of adverse event data. Thus, the information about adverse events may include (i) a set of adverse event data registered for one drug, or (ii) a set of occurrence frequency data for each adverse event calculated based on a set of adverse event data for one drug. The occurrence frequency data is linked with a label indicating a drug name that indicates to which drug the occurrence frequency data belongs.

Similarly, for indications as well, applicable diseases or symptoms, and diseases or symptoms for which applicability has not been reported are registered for each drug. In other words, for multiple diseases or symptoms, information indicating whether or not each disease or symptom is an indication is registered for each drug. In this description, information indicating whether or not one drug may be applicable to a certain disease or symptom is referred to as “indication data.” Indication data is linked with a label indicating a drug name that indicates to which drug the indication data belongs. In a drug database, multiple items of indication data are registered per drug, and these constitute a set of indication data. The information indicating whether or not a disease or symptom is an indication that is included in the training data is merely information registered in a drug database and may include information that has not been experimentally confirmed if the drug is actually applicable.

Here, the term “linked” is merely intended to mean that a label is attached so that the correspondence relationship between each item of data and a drug to which the data belongs can be understood. No label indicating a drug name is attached to the information about adverse events and the indication data to be input into an artificial intelligence.

In the upper part of FIG. 1, pieces of information about adverse events (AE1, AE2, AE3, AE4, . . . in FIG. 1) reported for individual known drugs (Drug 1, . . . in FIG. 1) can be linked with each item of indication data (Indication A: YES, Indication B: NO) for each drug based on, for example, labels indicating the drug names.

By way of example, FIG. 1 shows an example in which artificial intelligence models that do not have a neural network structure such as support vector machines (SVMs) are used.

In this example, one artificial intelligence model is used for one indication, and an artificial intelligence model is trained for each indication.

Thus, in order to predict the applicability to a predetermined indication (for example, Indication A), pieces of information about adverse events reported for individual known drugs (AE1, AE2, AE3, AE4, . . . in FIG. 1), and indication data corresponding to each drug (for example, Indication A: YES) are input in combination into one artificial intelligence model to train the artificial intelligence model. Similarly, in order to predict the applicability to another indication (for example, Indication B), pieces of information about adverse events reported for individual known drugs (AE1, AE2, AE3, AE4, . . . in FIG. 1), and indication data corresponding to each drug (for example, Indication B: No) are input in combination into one artificial intelligence model to train the artificial intelligence model.

The drugs may or may not include drugs for which test data that is used in the prediction phase is acquired.

### (2) Prediction Phase

Next, the trained artificial intelligence models are used to predict a new indication for a known drug of interest or its equivalent substance. In the prediction phase, information about adverse events related to a drug of interest for which an indication is desired to be predicted that is generated in the same manner as the information about adverse events that is acquired in the training phase is used as test data. The test data is input into the artificial intelligence models trained as in Section (1) above to predict an indication.

The lower part of FIG. 1 shows an example of a prediction phase. Here, Drug 1 used in the training phase is used as a drug for which a new indication is desired to be predicted. The pieces of information AE1, AE2, AE3, AE4, . . . about adverse events reported for Drug 1 used in the above training phase are respectively input into artificial intelligence models trained for each indication (SVM for Indication A, and SVM for Indication B in FIG. 1) as test data. Because Drug 1 has been originally reported to be effective against Indication A, a label “YES” indicating that there is applicability is output from the SVM for Indication A for predicting the applicability to Indication A. On the other hand, if a label “YES” is also output from the SVM for Indication B, it is predicted that Drug 1 may be applicable to Indication B, which has not been reported for Drug 1.

The “information about adverse events” used in the prediction phase is defined in the same manner as in (1) training phase.

This is due to the fact that public drug databases are constructed based on the results from actual clinical trials or the like and therefore diseases or symptoms that are not the targets of clinical trials or the like are registered as those to which there is no applicability.

In other words, according to a prediction method disclosed in this description, it is indicated that drug repositioning and/or drug repurposing can be achieved even when information about adverse events reported for drugs included in the training data is used as test data.

### (3) Description of Terms

In this disclosure, the term “drug” includes pharmaceutical products, quasi-pharmaceutical products, cosmeceutical products, foods, foods for specified health uses, foods with functional claims and candidates therefor. Also, the term “drug” includes substances whose tests were suspended or terminated during a preclinical trial or clinical trial for pharmaceutical approval. Here, the term “drug” may include single drugs and concomitant drugs in which multiple drugs are combined.

The “known drug” is not limited as long as it is an existing drug. Preferably, it is a drug with known effects on humans. Also, the term “equivalent substance of a drug” may include drugs that have a similar structure and a similar effect to an existing drug. The term “similar effect” here is intended to mean having the same kind of effect as a known drug although the intensity of the effect is different.

The “adverse event” is not limited as long as it is an effect that is determined to be harmful to humans. Preferred examples include adverse events listed in public drug databases such as FAERS (https://www.fda.gov/Drugs/GuidanceComplianceRegulatorylnformation/Surveillance/Adverse DrugEffects/ucm082193.htm) or clinicaltrials.gov (https://clinicaltrials.gov/).

The term “side effect” is intended to mean an effect on humans other than the indication for each drug, not limited to adverse events. Examples of the side effect include those listed in a public drug database such as SIDER4.1 (http://sideeffects.embl.de).

The occurrence frequency of an adverse event or side effect can be obtained by the following method. A word or phrase indicating the name of an adverse event is extracted by, for example, text extraction from a database as described above such as clinicaltrials.gov, FAERS, or all drug labels of DAILYMED. One extracted word or phrase can be counted as one reported adverse event. When an adverse event is taken as an example, for one known drug, the occurrence frequency can be obtained according to the equation: Occurrence frequency=(the number of cases reported for one adverse event)/(the total number of cases of adverse events reported for the known drug). When explanations related to effects are registered in text form in a database, syntactic analysis, word segmentation, semantic analysis or the like may be performed on the registered texts by natural language processing before the extraction of the texts corresponding to the effects.

The “indication” is not limited as long as it is a disorder or symptom in humans that should be mitigated, treated, arrested or prevented. Examples of the disorder or symptom include disorders or symptoms listed in a public drug database such as the above-mentioned FAERS, all drug labels of DAILYMED (https://dailymed.nlm.nih.gov/dailymed/spl-resources-all-drug-labels.cfm), Medical Subject Headings (https://www.nlm.nih.gov/mesh/meshhome.html), Drugs@FDA (https://www.accessdata.fda.gov/scripts/cder/daf/), or International Classification of Diseases (https://www.who.int/health-topics/international-classification-of-diseases). More specifically, examples of the indication include ischemic diseases such as thrombosis, embolism and stenosis (in particular, heart, brain, lungs, large intestine, etc.); circulatory disorders such as aneurysm, phlebeurysm, congestion and hemorrhage (aortae, veins, lungs, liver, spleen, retinae, etc.); allergic diseases such as allergic bronchitis and glomerulonephritis; dementia such as Alzheimer's dementia; degenerative diseases such as Parkinson's disease, amyotrophic lateral sclerosis and myasthenia gravis (nerves, skeletal muscles, etc.); tumors (benign epithelial tumor, benign non-epithelial tumor, malignant epithelial tumor, malignant non-epithelial tumor); metabolic diseases (abnormal carbohydrate metabolism, abnormal lipid metabolism, electrolyte imbalance); infectious diseases (bacteria, viruses, rickettsiae, chlamydiae, fungi, protozoa, parasites, etc.); and symptoms or illnesses associated with autoimmune diseases or the like such as renal diseases, systemic lupus erythematodes and multiple sclerosis.

In this disclosure, the term “artificial intelligence model” means a unit of algorithms that can output a result of interest from a set of input data. Examples of the artificial intelligence model may include support vector machine (SVM), relevance vector machine (RVM), naive Bayes, logistic regression, random forest, feedforward neural network, deep learning, K-nearest neighbor algorithm, AdaBoost, bagging, C4.5, Kernel approximation, stochastic gradient descent (SGD) classifier, Lasso, ridge regression, Elastic Net, SGD regression, kernel regression, LOWESS regression, matrix fractorization, nonnegative matrix fractorization, kernel matrix fractorization, interpolation, kernel smoother, and collaborative filtering.

In this disclosure, training an artificial intelligence model may include validation, generalization or the like. Examples of the validation and generalization include holdout method, cross-validation method, AIC (An Information Theoretical Criterion/Akaike Information Criterion), MDL (Minimum Description Length), and WAIC (Widely Applicable Information Criterion).

### 2. Construction of Artificial Intelligence Model

Construction of an artificial intelligence model is described using adverse events as an example.

### 2-1. Generation of Training Data

A method for generating training data is described. The training data includes information about adverse events reported for known drugs and indication data reported for the known drugs, which are generated based on information available from a public drug database 60.

For the definition of the terms “adverse event data”, “information about adverse events” and “indication data,” the description in Section 1(1) above is incorporated here.

Some drug databases, such as FAERS, basically include both adverse event data and indication data for each drug. In such a case, adverse event data reported for known drugs and indication data reported for the known drugs can be acquired from one drug database.

On the other hand, because only information about adverse events is described in, for example, clinicaltrials.gov or the like, the indications for each drug can be obtained from another drug database, such as FAERS, all drug labels of DAILYMED, Medical Subject Headings, Drugs@FDA, International Classification of Diseases or the like.

As described in Section 1(1) above, the adverse event data and indication data registered in a drug database are linked with labels indicating drug names so that one can understood to which drug each item of data belongs. The labels may be the drug names themselves or may be the registration numbers or the like of the drugs.

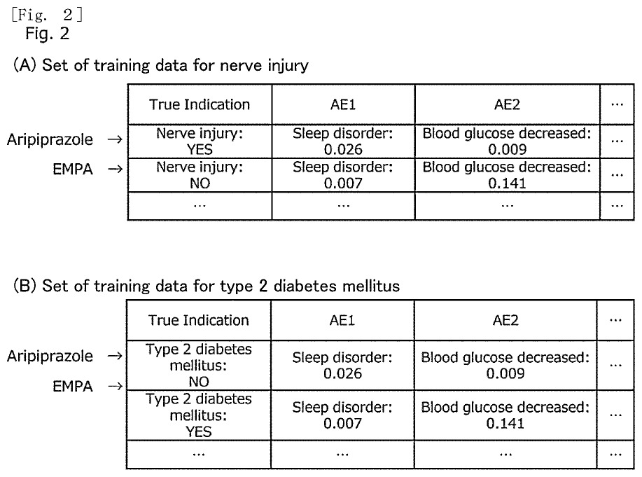

FIG. 2 shows examples of training data. FIG. 2(A) shows an example of a set of training data for nerve injury, and FIG. 2(B) shows a set of training data for type 2 diabetes mellitus. The names, such as Nerve injury and Type 2 diabetes mellitus, serve as labels indicating indication names. In FIG. 2, aripiprazole and empagliflozin (EMPA) are shown as examples of known drugs.

Aripiprazole and EMPA serve as labels indicating drug names. In FIG. 2, “True Indication” is intended to mean an indication against which the drug has been proved to be effective that is registered in a drug database. For example, “True Indication” is nerve injury in FIG. 2(A), and “True Indication” is type 2 diabetes mellitus in FIG. 2(B). Because aripiprazole is a drug that is applicable to nerve injury, “Nerve injury: YES” has been entered in the column of “True Indication” in FIG. 2(A). Because EMPA is a drug that is not applicable to nerve injury, “Nerve injury: NO” has been entered in the column of “True Indication.” Because aripiprazole is a drug that is not applicable to type 2 diabetes mellitus, “Type 2 diabetes mellitus: NO” has been entered in the column of “True Indication” in FIG. 2(B). Because EMPA is a drug that is applicable to type 2 diabetes mellitus, “Type 2 diabetes mellitus: YES” has been entered in the column of “True Indication.”

“Nerve injury: YES,” “Nerve injury: NO,” “Type 2 diabetes mellitus: NO,” and “Type 2 diabetes mellitus: YES” serve as items of indication data.

The labels indicating whether or not a drug is effective against an indication that have been registered in a drug database may be “Y” and “N,” “1” and “0,” “1” and “−1” or the like besides “YES” and “NO.”

As described in Section 1(1) above, multiple items of indication data are registered per drug in a drug database, and these constitute a set of indication data.

In FIG. 2, Sleep disorder and Blood glucose decreased are shown as examples of adverse events. In FIG. 2(A), “Sleep disorder:0.026” and “Blood glucose decreased:0.009” are contained in the row of aripiprazole. The values “0.026” and “0.009” represent the occurrence frequencies of the respective adverse events. Thus, “Sleep disorder:0.026” and “Blood glucose decreased: 0.009” serve as occurrence frequency data for the respective adverse events. Thus, “Sleep disorder:0.026” and “Blood glucose decreased:0.009” constitute information about adverse events related to aripiprazole. Thus, in the row of aripiprazole in FIG. 2(A), “Nerve injury: YES” as indication data is linked with “Sleep disorder:0.026” and “Blood glucose decreased:0.009” as information about adverse events. In other words, the combination of “Nerve injury: YES” with “Sleep disorder:0.026” and “Blood glucose decreased:0.009” linked therewith (which may be represented as [“Nerve injury: YES” “Sleep disorder:0.026”+“Blood glucose decreased:0.009” ]) constitutes one item of training data.

Also, in FIG. 2(A), “Sleep disorder:0.007” and “Blood glucose decreased:0.141” are contained in the row of EMPA. “Sleep disorder:0.007” and “Blood glucose decreased:0.141” constitute information about adverse events related to EMPA. Thus, a combination in which indication data “Nerve injury: NO” is linked with these pieces of information about adverse events (which may be represented as [“Nerve injury: NO”_“Sleep disorder:0.007”+“Blood glucose decreased:0.141” ]) constitutes one item of training data.

In FIG. 2(B), “Sleep disorder:0.026” and “Blood glucose decreased:0.009” are contained as information about adverse events in the row of aripiprazole. In FIG. 2(B), indication data for aripiprazole is “Type 2 diabetes mellitus: NO.” The combination of “Type 2 diabetes mellitus: NO” with the information about adverse events (which may be represented as [“Type 2 diabetes mellitus: NO”_“Sleep disorder:0.026”+“Blood glucose decreased:0.009” ]) constitutes one item of training data.

In FIG. 2(B), “Sleep disorder:0.007” and “Blood glucose decreased:0.141” are contained as information about adverse events in the row of EMPA. In FIG. 2(B), indication data for aripiprazole is “Type 2 diabetes mellitus: YES.” The combination of “Type 2 diabetes mellitus: NO” with the information about adverse events (which may be represented as [“Nerve injury: YES”_“Sleep disorder:0.007”+“Blood glucose decreased:0.141” ] constitutes one item of training data.

When the artificial intelligence models are artificial intelligence models that do not have a neural network structure such as support vector machines (SVMs), one artificial intelligence model is used for one indication, and one artificial intelligence model is trained for each indication.

Thus, a set of training data include [“Nerve injury: YES”_“Sleep disorder:0.026”+“Blood glucose decreased:0.009” ] and [“Nerve injury: NO” “Sleep disorder:0.007”+“Blood glucose decreased: 0.141” ].

When the artificial intelligence models are artificial intelligence models having a neural network structure, one artificial intelligence model is trained for multiple indications. In other words, one trained artificial intelligence model corresponds to prediction of multiple indications.

Thus, a set of training data includes [“Nerve injury: YES”+“Nerve injury: NO” “Sleep disorder: 0.026”+“Blood glucose decreased:0.009” ] and [“Type 2 diabetes mellitus: NO”+“Type 2 diabetes mellitus: YES”_“Sleep disorder:0.026”+“Blood glucose decreased:0.009” ]. The set of training data for artificial intelligence models having a neural network structure is not limited as long as information about adverse events related to multiple drugs is associated with a set of indication data for the multiple drugs.

For convenience sake, two drugs and two adverse events are shown as examples in FIG. 2, and two items of indication data are respectively shown in FIG. 2(A) and FIG. 2(B) as examples. To increase predictable indications, it is preferred to use as many types of drugs as possible and adverse events data and indication data corresponding thereto.

The drug is not limited as long as it is a drug with which adverse event data and indication data can be linked in a drug database as described above. The number of drugs is preferably 1,000 or more, 2,000 or more, 3,000 or more, or 4,000 or more. The upper limit is the number of drugs registered in the drug database.

The number of items of indication data registered per drug is preferably 1,000 or more, 5,000 or more, or 10,000 or more. The upper limit is the number of items of indication data registered in the drug database.

The number of items of adverse event data registered per drug is preferably 1,000 or more, 5,000 or more, or 10,000 or more. The upper limit is the number of items of adverse event data registered in the drug database.

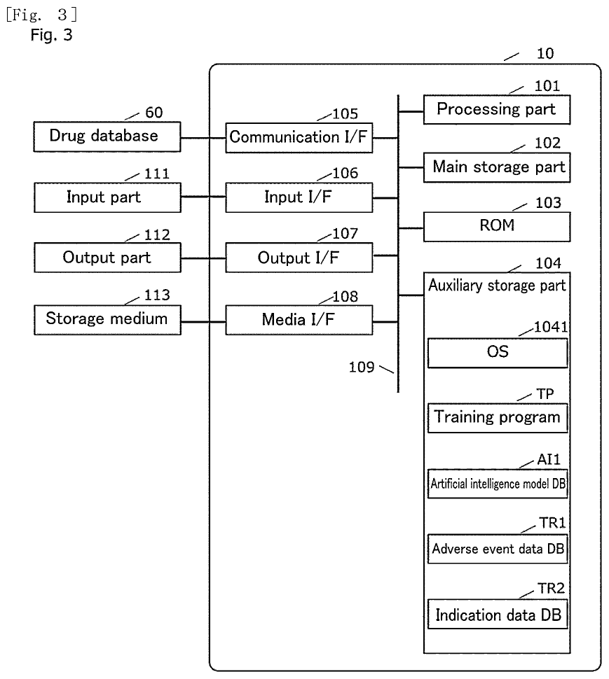

For the acquisition of adverse event data or a set of adverse event data from the drug database 60 shown in FIG. 3, a processing part 101 of a training device 10 starts the acquisition via a communication I/F 105 when the processing part 101 accepts a request to acquire data from an operator. The adverse event data and the set of adverse event data acquired are recorded in an adverse event database (DB) TR1 stored in an auxiliary storage part 104 by the processing part 101. Also, for the acquisition of indication data and a set of indication data from the drug database 60 shown in FIG. 3 as well, the processing part 101 of the training device 10 starts the acquisition via the communication I/F 105 when the processing part 101 accepts a request to acquire data from the operator. The indication data and the set of indication data acquired are recorded in an indication database (DB) TR2 of the auxiliary storage part 104 shown in FIG. 3 by the processing part 101.

### 2-2. Device for Training Artificial Intelligence Model

The training of an artificial intelligence model as described above can be achieved using, for example, the training device 10 (which is hereinafter referred to also as “device 10”).

In the description of the device 10 and the processing in the device 10, for the terms that are common to those described in Sections 1. and 2-1. above, the above description is incorporated here.

FIG. 3 illustrates a hardware configuration of the device 10. The device 10 includes at least the processing part 101 and a storage part. The storage part is constituted of a main storage part 102 and/or an auxiliary storage part 104. The device 10 may be connected to an input part 111, an output part 112, and a storage medium 113. Also, the device 10 is communicably connected to the drug database 60 such as FAERS, all drug labels of DAILYMED, Medical Subject Headings, Drugs@FDA, International Classification of Diseases, or clinicaltrials.gov.

In the device 10, the processing part 101, the main storage part 102, a ROM (read only memory) 103, the auxiliary storage part 104, the communication interface (I/F) 105, an input interface (I/F) 106, an output interface (I/F) 107, and a media interface (I/F) 108 are connected for mutual data communication by a bus 109.

The processing part 101 is constituted of a CPU, MPU, GPU or the like. The processing part 101 executes a computer program stored in the auxiliary storage part 104 or the ROM 103 and processes the acquired data, whereby the device 10 functions. The processing part 101 trains an artificial intelligence model using training data as described in Section 1. above.

The ROM 103 is constituted of a mask ROM, a PROM, an EPROM, an EEPROM or the like, and stores computer programs that are executed by the processing part 101 and data that are used thereby. The ROM 103 stores a boot program that is executed by the processing part 101 when the device 10 is started up, and programs and settings relating to the operation of the hardware of the device 10.

The main storage part 102 is constituted of a RAM (Random access memory) such as an SRAM or DRAM. The main storage part 102 is used to read out the computer programs stored in the ROM 103 and the auxiliary storage part 104. The main storage part 102 is also utilized as a workspace when the processing part 101 executes these computer programs. The main storage part 102 temporarily stores training data or the like acquired via a network, functions of the artificial intelligence model read out by the auxiliary storage part 104, and so on.

The auxiliary storage part 104 is constituted of a hard disk, a semiconductor memory element such as a flash memory, an optical disk, or the like. In the auxiliary storage part 104, various computer programs to be executed by the processing part 101 such as an operating system and application programs, and various setting data for use in executing the computer programs are stored. Specifically, the auxiliary storage part 104 stores operation software (OS) 1041, a training program TP, an artificial intelligence model database (DB) AI1, an adverse event database (DB) TR1 for storing adverse event data for drugs and/or occurrence frequency data for adverse events and information about adverse events acquired from the drug database 60, and an indication database (DB) TR2 for storing indication data for drugs acquired from the drug database 60 in a non-volatile manner. The training program TP performs processing for training an artificial intelligence model as described later in corporation with the operation software (OS) 1041. In the artificial intelligence model database AI1, untrained and trained artificial intelligence models may be stored.

The communication I/F 105 is constituted of a serial interface such as a USB, IEEE1394 or RS-232C, a parallel interface such as an SCSI, IDE or IEEE1284, and an analog interface constituted of a D/A converter, A/D converter or the like, a network interface controller (NIC) and so on. The communication I/F 105, under the control of the processing part 101, receives data from a measurement part 30 or other external devices, and, when necessary, transmits information stored in or generated by the device 10 to the measurement part 30 or to the outside, or displays it.

The communication I/F 105 may communicate with the measurement part 30 or other external devices (not shown, e.g., other computers or cloud systems) via a network.

The input I/F 106 is constituted of a serial interface such as a USB, IEEE1394 or RS-232C, a parallel interface such as an SCSI, IDE or IEEE1284, an analog interface constituted of a D/A converter, A/D converter or the like, and so on. The input I/F 106 accepts character input, clicks, sound input or the like from the input part 111. The accepted inputs are stored in the main storage part 102 or the auxiliary storage part 104.

The input part 111 is constituted of a touch panel, keyboard, mouse, pen tablet, microphone or the like, and performs character input or sound input into the device 10. The input part 111 may be externally connected to the device 10 or may be integrated with the device 10.

The output I/F 107 is constituted, for example, of an interface similar to that for the input I/F 106. The output I/F 107 outputs information generated by the processing part 101 to the output part 112. The output I/F 107 outputs information generated by the processing part 101 and stored in the auxiliary storage part 104 to the output part 112.

The output part 112 is constituted, for example, of a display, a printer or the like, and displays measurement results transmitted from the measurement part 30, various operation windows in the device 10, respective items of training data, an artificial intelligence model, and so on.

The media I/F 108 reads out, for example, application software or the like stored in the storage medium 113. The read out application software or the like is stored in the main storage part 102 or the auxiliary storage part 104. Also, the media I/F 108 writes information generated by the processing part 101 into the storage medium 113. The media I/F 108 writes information generated by the processing part 101 and stored in the auxiliary storage part 104 into the storage medium 113.

The storage medium 113 is constituted of a flexible disk, a CD-ROM, a DVD-ROM or the like. The storage medium 113 is connected to the media I/F 108 by a flexible disk drive, a CD-ROM drive, a DVD-ROM drive or the like. An application program or the like for a computer to execute an operation may be stored in the storage medium 113.

The processing part 101 may acquire application software and various settings necessary for control of the device 10 via a network instead of reading them out of the ROM 103 or the auxiliary storage part 104. It is also possible that the application program is stored in an auxiliary storage part of a server computer on a network and the device 10 accesses this server computer to download the computer program and stores it in the ROM 103 or the auxiliary storage part 104.

Also, in the ROM 103 or the auxiliary storage part 104, an operation system that provides a graphical user interface environment, such as Windows (trademark) manufactured and sold by Microsoft Corporation in the United States, has been installed, for example. The training program TP shall operate on the operating system. In other words, the device 10 may be a personal computer or the like.

### 2-3. Processing by Training Program

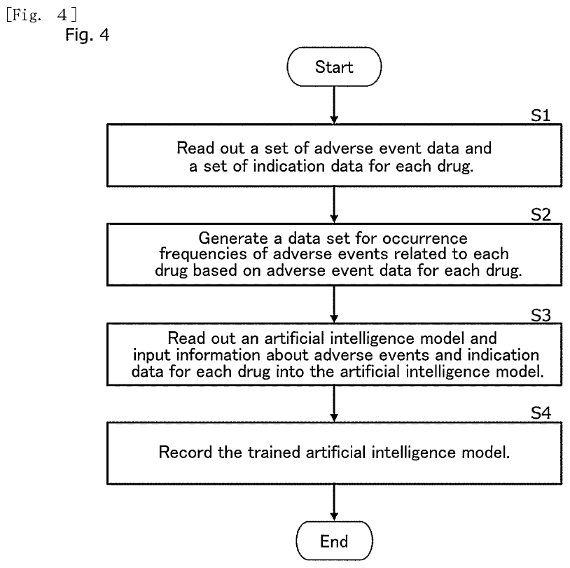

Referring to FIG. 4, the flow of processing for training an artificial intelligence model is described.

The processing part 101 accepts a request to start processing input by an operator through the input part 111, and, in step S1, reads out a set of adverse event data and a set of indication data for each drug from the database TR1 and the database TR2, respectively, stored in the auxiliary storage part 104.

In step S2, when necessary, the processing part 101 generates a data set for occurrence frequencies from the set of adverse event data for each drug. The method for calculating an occurrence frequency is as described in Section 1.(3) above.

In step S3, the processing part 101 generates information about adverse events for each drug according to the method described in Section 2-1. above. Also, the processing part 101 reads out an artificial intelligence model from the artificial intelligence model database AI1 stored in the auxiliary storage part 104, and inputs the generated information about adverse events and a set of indication data linked with the generated information about adverse events into the artificial intelligence model to train the artificial intelligence model. Here, the artificial intelligence model read out in step S3 may be an artificial intelligence model that has not been trained yet or an artificial intelligence model that has been already trained.

The processing part 101 records the trained artificial intelligence model into the auxiliary storage part 104 in step S4, and terminates the processing.

The training of an artificial intelligence model can be carried out using, for example, software such as Python.

### 3. Prediction of Indication by Artificial Intelligence Model

Taking adverse events as an example, prediction of an indication is described.

### 3-1. Generation and Recording of Test Data

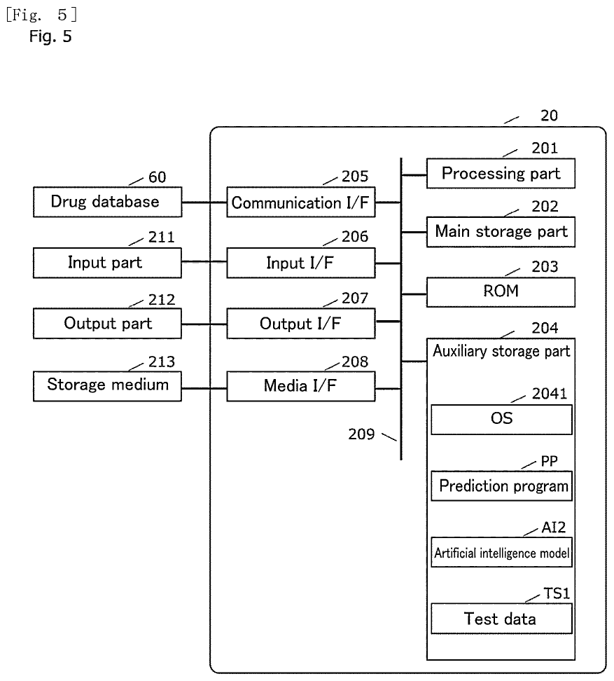

Test data is generated according to a method for generating information about adverse events as described in Section 2-1. above. At this time, for the acquisition of adverse event data and a set of adverse event data from the drug database 60 shown in FIG. 5, a processing part 201 of a prediction device 20 starts the acquisition via a communication I/F 205 when the processing part 201 accepts a request to acquire data from an operator. The processing part 201 records the adverse event data and the set of adverse event data acquired in a test data database TS1 (which may be hereinafter referred to simply as “database TS1”) stored in an auxiliary storage part 204.

Alternatively, the prediction device 20 may acquire at least one selected from adverse event data, a set of adverse event data and information about adverse events for a drug of interest from the adverse event database TR1 recorded in the auxiliary storage part 104 of the device 10 described in FIG. 3 via a network or a storage medium 213 and record it in the database TS1 in the auxiliary storage part 204.

### 3-2. Device for Predicting Indication

Prediction of an indication can be achieved using the prediction device 20 (which may be hereinafter referred to simply as “device 20”), for example.

In the description of the device 20 and the processing in the device 20, for the terms that are the common to those described in Sections 1. and 2-1. above, the above description is incorporated here.

FIG. 5 illustrates a hardware configuration of the prediction device 20 (which may be hereinafter referred to also as “device 20”). The device 20 includes at least the processing part 201 and a storage part. The storage part is constituted of a main storage part 202 and/or the auxiliary storage part 204. The device 20 may be connected to an input part 211, an output part 212, and a storage medium 213. Also, the device 20 is communicably connected to the drug database 60 such as FAERS, all drug labels of DAILYMED, Medical Subject Headings, Drugs@FDA, International Classification of Diseases, or clinicaltrials.gov. Further, the device 20 may be communicably connected to the device 10 via a network.

In the device 20, the processing part 201, the main storage part 202, a ROM (read only memory) 203, the auxiliary storage part 204, the communication interface (I/F) 205, an input interface (I/F) 206, an output interface (I/F) 207, and a media interface (I/F) 208 are connected for mutual data communication by a bus 209.

Because the basic hardware configuration of the device 20 is the same as that of the device 10, the description in Section 2-2 above is incorporated here.

However, in the auxiliary storage part 204 of the device 20, operation software (OS) 2041, a prediction program PP, an artificial intelligence model database AI2 for storing atrained artificial intelligence model, and a database TS1 for storing test data are stored in a non-volatile manner in place of the operation software (OS) 1041, the training program TP, the artificial intelligence model database AI1, the adverse event database TR1 and the indication database TR2. The prediction program PP performs processing for predicting an indication as described later in cooperation with the operation software (OS) 2041. Here, the trained artificial intelligence model is one that has been acquired from the auxiliary storage part 104 of the device 10 described in FIG. 3 via a network or the storage medium 213 and recorded in the auxiliary storage part 204 by the processing part 201 of the prediction the device 20.

### 3-3. Processing by Prediction Program

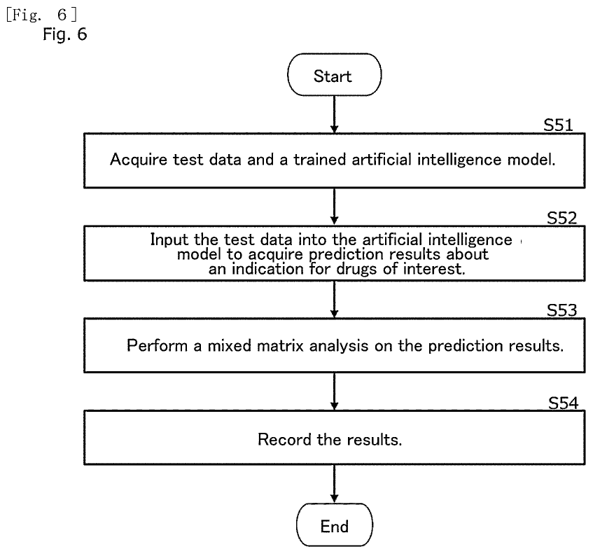

Referring to FIG. 6, the flow of processing for predicting an indication is described.

The processing part 201 accepts a request to start processing input by an operator through the input part 211, and, in step S51 of FIG. 6, read outs test data from the database TS1 stored in the auxiliary storage part 204. Also, the processing part 201 reads out a trained artificial intelligence model from the artificial intelligence model database AI2 stored in the auxiliary storage part 204.

Next, the processing part 201 accepts a request to start prediction input by the operator through the input part 211, and, in step S52, inputs the test data into the trained artificial intelligence model to acquire prediction results about an indication for a drug of interest. The prediction result may be output from the trained artificial intelligence model as a combination of a label indicating an indication name with a label indicating whether or not the indication is an indication for a drug of interest. As a label indicating whether or not the indication is an indication for the drug of interest, “1” can be output when the drug of interest is predicted to be “effective” against the corresponding indication by the artificial intelligence model and “0” or “−1” can be output when it is determined to be “ineffective.” For example, when the indication is “Nerve injury” and when the drug of interest is predicted to be effective against nerve injury, “Nerve injury:1” is output as a prediction result. When the drug of interest is predicted to be ineffective against nerve injury, “Nerve injury:0 or “Nerve injury:−1” is output as a prediction result. The processing part 201 records these prediction results into the auxiliary storage part 204.

Next, the processing part 201 accepts a request to analyze prediction results input by the operator through the input part 211, and, in step S54, performs a mixed matrix analysis on the prediction results acquired in step S53 to determine whether the prediction result for an indication output for each drug is true positive (TP) or false positive (FP). When the result is true positive, a label “1” is attached to the label indicating the indication name, for example. When the result is false positive, a label “0” is attached to the label indicating the indication name, for example. True positive means that the indication is registered as an “indication” (against which the drug is effective) for each drug registered in the drug database 60, and is also predicted as an “indication” therefor in a prediction result. False positive means that the indication is not registered as an “indication” for each drug registered in the drug database 60 but is predicted as an “indication” in a prediction result. The indication determined to be false positive will be a new indication for the drug of interest. Specifically, as described in Section 1. and 2-1. above, the indication data for each drug has a label indicating an indication name and a label indicating whether or not each drug is effective against the indication attached thereto. For example, when the prediction result is “Nerve injury:1” even though the indication data is “Nerve injury:0” or “Nerve injury:−1,” the indication can be determined as being false positive. When the indication data is “Nerve injury: 1” and the prediction result is “Nerve injury:1,” the indication is true positive.

Next, the processing part 201 accepts a request to record the analysis results input by the operator through the input part 211, and, in step S55, records the analysis results from step S54 into the auxiliary storage part 204 and then terminates the processing.

After step S55, the processing part 201 may accept a request to output input by the operator through the input part 211 or may be triggered by the completion of step S55 to output the analysis results to the output part 212.

The prediction processing can be carried out using, for example, software such as Python.

The mixed matrix analysis can be carried out using, for example, software “R.”

### 4. Computer Program

### 4-1. Training Program

The computer program is a computer program that causes a computer to execute the processing including steps S1 to S4 in FIG. 6 described above in connection with training of an artificial intelligence model to cause the computer to function as the training device 10.

### 4-2. Prediction Program

The compute program is a computer program that causes a computer to execute the processing including steps S51 to S54 described above in connection with prediction of effects of a test substance to cause the computer to function as the prediction device 20.

### 5. Storage Medium Having Computer Programs Stored Therein

This section relates to a storage medium having the above computer programs stored therein. The computer programs are stored in a storage medium such as a hard disk, a semiconductor memory element such as a flash memory, or an optical disk. Also, the computer programs may be stored in a storage medium connectable via a network such as a cloud server. The computer programs may be program products that are in a downloadable form or stored in a storage medium.

The storage format of the programs in the storage medium is not limited as long as a device as described above can read the programs. The storage in the storage medium is preferably in a non-volatile manner.

### 6. Modifications

In this description, the same reference numeral attached to hardware indicates the same part or same function.

In Section 2. above, an embodiment is shown in which the training device 10 and the prediction device 20 are different computers. However, one computer may perform training of an artificial intelligence model and prediction.

In Sections 1. to 5. above, information about adverse events is used for the explanation of training of an artificial intelligence model and indication prediction. However, side effects may be used instead of adverse events. In this case, the term “adverse events” in each device, each processing and each method can be replaced by the term “side effects” except for the definition of the terms.

### 7. Composition

This description includes, as a certain embodiment of the present invention, a composition for using a drug selected from a drug list as described later in treatment or prevention of each indication listed correspondingly to each drug in the drug list. The indication corresponding to each drug listed in the drug name list is a new indication predicted by the above prediction method.

Here, the term “treatment” can include improving or curing. The term “prevention” includes inhibiting the onset, recurrence or progression of an indication.

The drugs listed in the drug list are known drugs. Thus, administration methods and so on are known. Also, because the adverse event data and indication data used in the prediction method are based on events observed when each drug is administered under normal dose and usage, the dose and usage in using each drug against a new indication can be determined in reference to the known dose range and usage.

The composition can be prepared in combination with appropriate carriers and additives in addition to a drug shown in the drug list. Examples of the carriers and additives for use in the preparation of the composition are those commonly used in drugs and include excipients, binders, disintegrators, lubricants, colorants, flavoring substances, odorants, and surfactants.

### 8. Verification of Effects of Artificial Intelligence Model (1) Training of Artificial Intelligence Model, and Evaluation of Performance of Trained Artificial Intelligence Model

For all drugs reported to the U.S. Food & Drug Adverse Event Reporting System (FAERS) from the third quarter of 2014 to the fourth quarter of 2017, all occurrence frequency data for adverse events and all indication data registered for each drug were acquired. There are 11,310 indications. Specifically, for 4,885 drugs, a data set including a set of occurrence frequency data and a set of indication data was acquired.

Using all the data, an SVM was trained for each indication according to the generation of training data as described in Section 2-1. above to generate a trained artificial intelligence model.

Occurrence frequency data for 17,155 adverse events registered for respective 4,885 drugs registered in FAERS was individually calculated to generate a set of occurrence frequency data for adverse events for each drug. The sets of occurrence frequency data for adverse events for respective drugs were individually input as test data into the trained artificial intelligence model to perform prediction of indications.

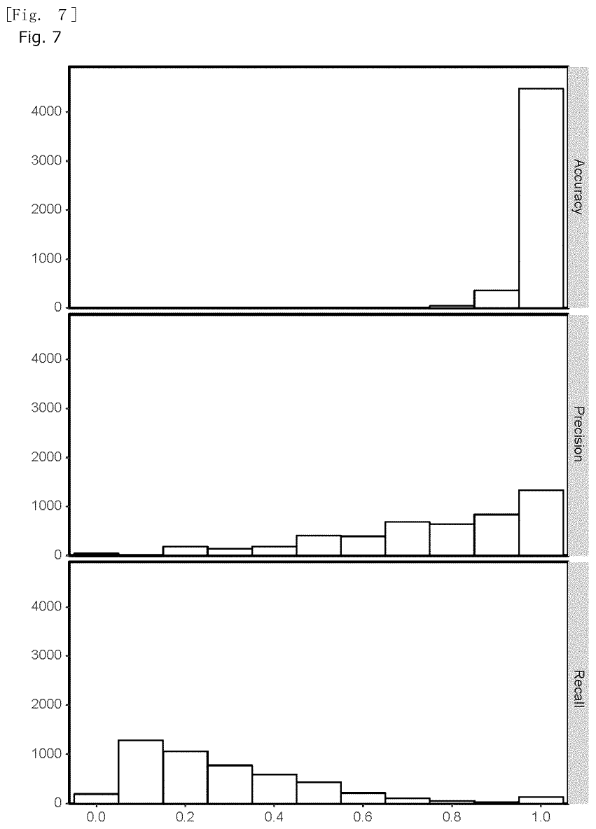

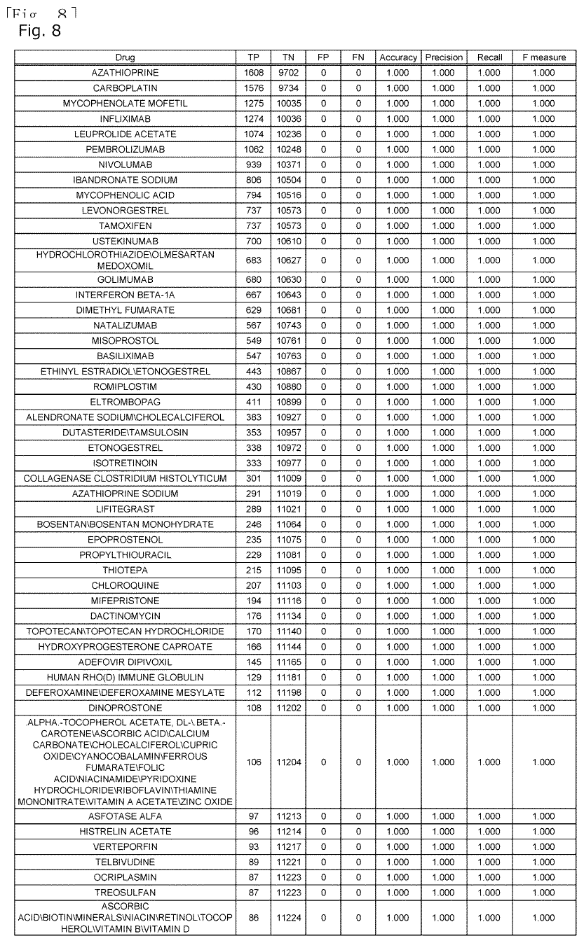

The results are shown in FIG. 7 to FIG. 10. FIG. 7 and FIG. 8 show results showing how accurately the indications reported for respective drugs were able to be predicted.

FIG. 7 shows, for all drugs, the distributions of accuracy score, which indicates the accuracy of prediction, recall score, which indicates the coverage in the case of being predicted as an “indication,” and precision score, which indicates the reliability in the case of being predicted as an “indication,” in rod graphs. The accuracy score and the precision score are more accurate as they are closer to 1.0. The correctness of an indication against which the drug is reported to be “effective” is intended to approach 100% as the recall score is closer to 1.

The vertical axes of the graphs show the number of drugs that belong to each quantile when the score ranging from −0.1 to 1.0 is divided into 11 quantiles of 0.1.

For all drugs input as test data into the trained artificial intelligence model, the accuracy score of the results of prediction of indications was as high as not lower than 90% for 4,764 drugs out of 4,885 drugs (97.5%).

Out of 4,885 drugs, 1,790 drugs (36.6% of all drugs) showed a precision score of 90% or higher, 3,252 drugs (66.6% of all drugs) showed a precision score of 70% or higher, and 4,238 drugs (86.8% of all drugs) showed a precision score of 50% or higher.

Out of 4,885 drugs, 746 drugs (15.3% of all drugs) showed a recall score of 50% or higher, 1,951 drugs (39.9% of all drugs) showed a recall score of 30% or higher, and 4,092 drugs (83.8% of all drugs) showed a recall score of 10% or higher.

FIG. 8 shows respective scores of the top 50 drugs having accuracy, precision and recall scores that are all 1.0 among the 4,885 drugs. In FIG. 8, TN represents true negative, TP represents true positive, FN represents false negative, and FP represents true positive. True negative indicates the number of items that were able to be predicted as not being indications for those that are not indications, and true positive indicates the number of items that were able to be predicted as being indications for those that are indications. False negative indicates the number of items that were predicted as being not indications for those that are indications, and false positive indicates the number of items that were predicted as being indications for those that are not indications. The F-measure score is a harmonic mean between the precision score and the recall score, and is an index for evaluating how much accuracy is obtained when the precision score and the recall score are integrated.

Also, the TN, TP, FN, FP, accuracy score, precision score, recall score and F-measure score of all drugs are shown as a data list 1 at the end of the Detailed Description of the Invention.

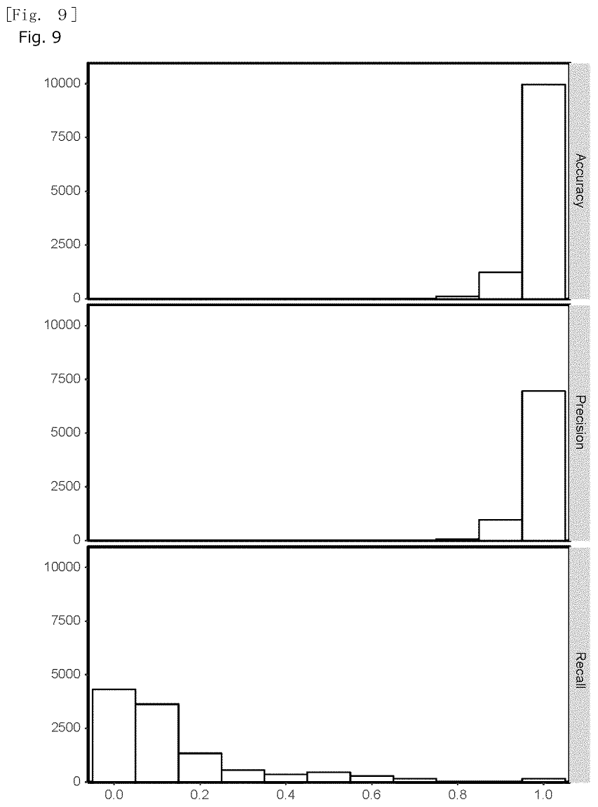

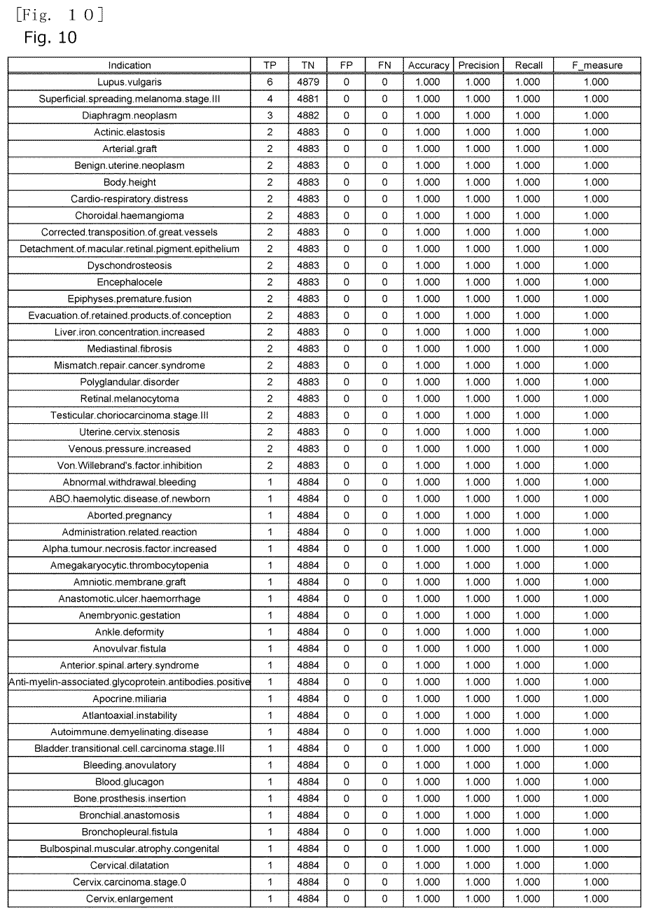

FIG. 9 and FIG. 10 show results showing how accurately the results of prediction of indications derived from the trained artificial intelligence model predicted each indication reported (registered in FAERS).

FIG. 9 shows, for all indications, the distributions of accuracy score, recall score, and precision score in rod graphs. The configuration of the graphs is the same as FIG. 7.

For all reported indications, the accuracy score of the prediction results was as high as not lower than 90% for 10,929 indications out of 11,310 indications (96.6%).

Out of 11,310 indications, 7,230 indications (63.9% of all TIs) showed a precision score of 90% or higher, and 8,016 indications (70.9% of all TIs) showed a precision score of 80% or higher.

Out of 11,310 indications, 972 indications (8.6% of all TIs) showed a recall score of 50% or higher, 1,786 indications (15.8% of all TIs) showed a recall score of 30% or higher, and 4,873 indications (43.1% of all TIs) showed a recall score of 10% or higher.

FIG. 10 shows respective scores of top 50 indications having accuracy, precision and recall scores that are all 1.0 among the 11,310 indications. The terms used in FIG. 10 are the same as those in FIG. 8.

Also, the TN, TP, FN, FP, accuracy score, precision score, recall score, and F-measure score of all indications are shown as a data list 2 at the end of Detailed Description of the Invention.

The above evaluation results indicate that the trained artificial intelligence model disclosed in this description can predict indications from information about adverse events.

### (2) Blind Evaluation Using Trained Artificial Intelligence Model

Next, it was evaluated whether accurate prediction can be made using information about adverse events that are not included in a set of training data.

The drugs used for training of an artificial intelligence model in Section 8.(1) above include drugs approved by U.S. Food and Drug Administration (FDA) and/or Pharmaceuticals and Medical Devices Agency (PMDA) from 2017 to 2019, and 61 drugs reported by repositioning by Perwitasari et al. (Non-Patent Document 2).

Thus, in the blind evaluation of an artificial intelligence model, an SVM was trained in the same manner as described in Section 8.(1) above using a set of training data which does not include information about adverse events and a set of indication data of the 61 drugs.

Next, the information about adverse events related to the 61 drugs was input into the trained artificial intelligence model, and prediction of indications was performed in the same manner as described in Section 7.(1) above.

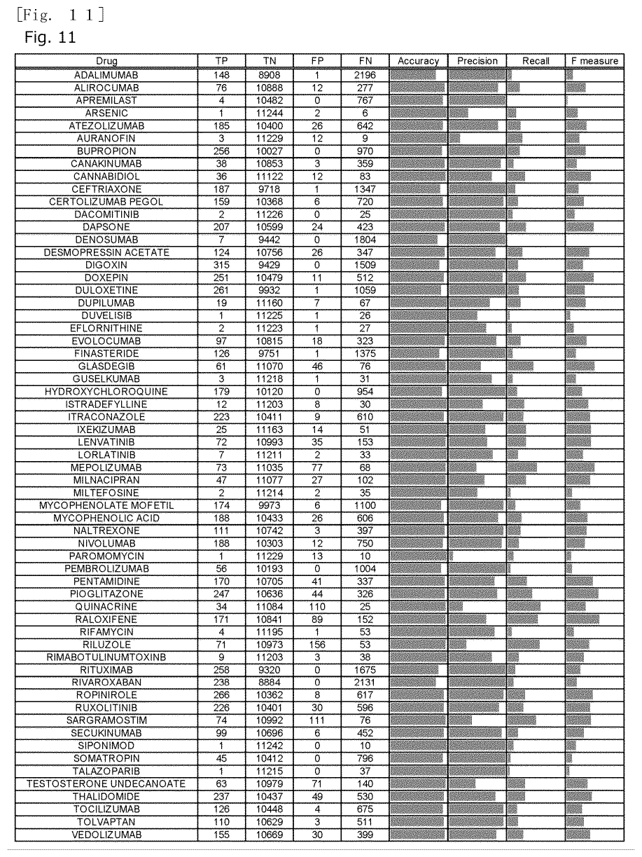

The results are summarized in FIG. 11. The terms used in FIG. 11 have the same meaning as those in FIG. 8.

Out of the 61 drugs, 54 drugs (88.5% of the drugs) showed an accuracy score of 90% or higher. Out of the 61 drugs, 27 drugs (44.3%) showed a precision score of 90% or higher, 44 drugs (72.1%) showed a precision score of 70% or higher, 53 drugs (86.9%) showed a precision score of 50% or higher. Out of the 61 drugs, 4 drugs (6.6%) showed a recall score of 50% or higher, 17 drugs (27.9%) showed a recall score of 30% or higher, and 45 drugs (73.8%) showed a recall score of 10% or higher.

These results indicate that prediction of indications can be made for drugs that are not included in a set of training data with accuracy guaranteed.

(3) Comparison of Performance with Prior Art

Non-Patent Document 4 (Li and Lu) reports a method for predicting an indication using machine learning. Thus, performance was compared between the method reported by Li and Lu and the prediction method according to this embodiment. The method reported by Li and Lu utilizes fingerprinting/target/mutual effect information of drugs and utilizes a potential relationship between side effects and indications.

The parallel comparison between the method reported by Li and Lu and the prediction method according to this embodiment was made using 15 drugs (bupropion, ceftriaxone, dapsone, digoxin, doxepin, finasteride, hydroxychloroquine, itraconazole, mycophenolate mofetil, mycophenolic acid, naltrexone, paromomycin, pioglitazone, riluzole, and ropinirole) that satisfies the following two criteria. The two conditions are as follows. 1) The drug must be present in both the prediction result obtained according to this embodiment and the prediction result obtained using fingerprinting/target/mutual effect information, and 2) A TIERS label for the drug is available in both the FAERS database and the SIDER database.

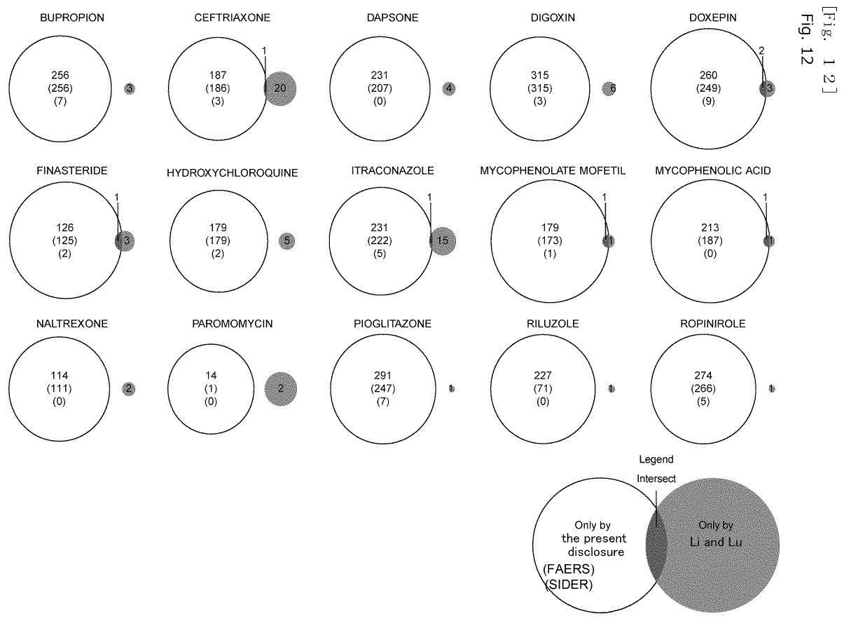

FIG. 12 shows the results as Venn diagrams. In FIG. 12, white circles indicate prediction results obtained according to this embodiment, and gray circles indicate prediction results by the method reported by Li and Lu.

It was demonstrated that indications that cannot be predicted by the method reported by Li and Lu can be predicted by the prediction method of this embodiment.

(4) Drugs with Predicted New Indication and their Indications

Drugs that show “NO” in indication data in a drug database but that showed “YES” (potential applicability) for an indication by the above prediction method and their indications are shown in a drug list. The numerical value in the right column of the drug list indicates “importance” in the SVM. The “importance” indicates how far away the indication is from the decision boundary at which the SVM determines YES or NO. The higher the importance is, the more reliable the prediction is.

<Drug list>

The predicted new indication is shown for each drug in the following order: (Drug:indication:importance).

(AMPHETAMINE ASPARTATE\AMPHETAMINE SULFATE\DEXTROAMPHETAMINE SACCHARATE\DEXTROAMPHETAMINE SULFATE:5q minus syndrome:0.42),(ARMODAFINIL:5q minus syndrome:0.06), (BECLOMETHASONE DIPROPIONATE:5q minus syndrome:0.08), (BISOPROLOL FUMARATE:5q minus syndrome:0.29), (BORTEZOMIB:5q minus syndrome:0.27), (CALCIUM CARBONATE\CHOLECALCIFEROL:5q minus syndrome:0.29), (CANDESARTAN:5q minus syndrome:0.01), (CANDESARTAN CILEXETIL:5q minus syndrome:0.2), (CARVEDILOL PHOSPHATE:5q minus syndrome:0.02), (CELECOXIB:5q minus syndrome:0.39), (CERTOLIZUMAB PEGOL:5q minus syndrome:0.32), (CLINDAMYCIN\CLINDAMYCIN PHOSPHATE:5q minus syndrome:0.24), (CLONIDINE:5q minus syndrome:0.15), (DEXLANSOPRAZOLE:5q minus syndrome:1.31), (DIPHENHYDRAMINE:5q minus syndrome:0.42), (DIVALPROEX SODIUM:5q minus syndrome:0.3), (DOXAZOSIN\DOXAZOSIN MESYLATE:5q minus syndrome:0.23), (EPINEPHRINE:5q minus syndrome:0.09), (EVEROLIMUS:5q minus syndrome:0.06), (EVOLOCUMAB:5q minus syndrome:0.06), (FLECAINIDE:5q minus syndrome:0.08), (GLYBURIDE:5q minus syndrome:0.05), (HYOSCYAMINE:5q minus syndrome:0.08), (IBRUTINIB:5q minus syndrome:0.81), (IMATINIB MESYLATE:5q minus syndrome:0.07), (LINAGLIPTIN:5q minus syndrome:0.13), (LISDEXAMFETAMINE DIMESYLATE:5q minus syndrome:0.18), (METAXALONE:5q minus syndrome:0.56), (METRONIDAZOLE:5q minus syndrome:0.46), (NILOTINIB:5q minus syndrome:0.01), (OMEGA-3-ACID ETHYL ESTERS:5q minus syndrome:0.34), (OXYCODONE:5q minus syndrome:0.33), (PANTOPRAZOLE MAGNESIUM:5q minus syndrome:0.07), (POLYETHYLENE GLYCOLS:5q minus syndrome:0.25), (PONATINIB:5q minus syndrome:0.28), (PRAVASTATIN SODIUM:5q minus syndrome:0.84), (SACUBITRIL\VALSARTAN:5q minus syndrome:0.09), (SODIUM OXYBATE:5q minus syndrome:0.45), (SULFAMETHOXAZOLE:5q minus syndrome:0.14), (SUMATRIPTAN\SUMATRIPTAN SUCCINATE:5q minus syndrome:0.08), (.ALPHA.1-PROTEINASE INHIBITOR HUMAN:Abdominal discomfort:0.83), (ACENOCOUMAROL:Abdominal discomfort:0.97), (ACETAMINOPHEN\PROPOXYPHENE HYDROCHLORIDE:Abdominal discomfort:0.65), (ACETAMINOPHEN\PROPOXYPHENE NAPSYLATE:Abdominal discomfort:0.74), (ACITRETIN:Abdominal discomfort:0.53), (ALBIGLUTIDE:Abdominal discomfort:0.57), (ALCOHOL:Abdominal discomfort:1.04), (ALDESLEUKIN:Abdominal discomfort:0.08), (ALENDRONIC ACID:Abdominal discomfort:1.45), (ALFUZOSIN HYDROCHLORIDE:Abdominal discomfort:0.74), (AMINO ACIDS\DEXTROSE\ELECTROLYTES NOS:Abdominal discomfort:0.67), (AMISULPRIDE:Abdominal discomfort:1.23), (AMLODIPINE\PERINDOPRIL:Abdominal discomfort:0.12), (AMMONIUM CHLORIDE\DEXTROMETHORPHAN\SODIUM CITRATE:Abdominal discomfort:0.25), (AMMONIUM LACTATE:Abdominal discomfort:0.84), (AMOXICILLIN\CLARITHROMYCIN\ESOMEPRAZOLE:Abdominal discomfort:0.16), (ANAGRELIDE HYDROCHLORIDE:Abdominal discomfort:0.14), (ANTIHISTAMINES NOS:Abdominal discomfort:0.51), (ARGININE:Abdominal discomfort:0), (ARIPIPRAZOLE LAUROXIL:Abdominal discomfort:0.11), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Abdominal discomfort:0.24), (ASPIRIN\MAGNESIUM OXIDE:Abdominal discomfort:0.05), (ATROPINE SULFATE:Abdominal discomfort:0.25), (AZELNIDIPINE:Abdominal discomfort:0.3), (BALSALAZIDE DISODIUM:Abdominal discomfort:0.23), (BETAMETHASONE DIPROPIONATE\CALCIPOTRIENE\CALCIPOTRIENE HYDRATE:Abdominal discomfort:0.92), (BETAMETHASONE\CALCIPOTRIENE:Abdominal discomfort:0.76), (BLINATUMOMAB:Abdominal discomfort:0.42), (BUPRENORPHINE HYDROCHLORIDE:Abdominal discomfort:1.12), (CABOZANTINIB S-MALATE:Abdominal discomfort:0.4), (CALCIPOTRIENE:Abdominal discomfort:0.95), (CALCIUM CARBONATE\ERGOCALCIFEROL\RETINOL:Abdominal discomfort:0.37), (CALCIUM\MAGNESIUM:Abdominal discomfort:0.97), (CANNABIS SATIVA SUBSP. INDICA TOP:Abdominal discomfort:0.57), (CAPSAICIN:Abdominal discomfort:0.68), (CARBIDOPA:Abdominal discomfort:0.62), (CASPOFUNGIN:Abdominal discomfort:0.06), (CEFEPIME\CEFEPIME HYDROCHLORIDE:Abdominal discomfort:0.1), (CEFTAZIDIME:Abdominal discomfort:0.34), (CERITINIB:Abdominal discomfort:0.63), (CHLORHEXIDINE GLUCONATE:Abdominal discomfort:0.65), (CHLORPHENIRAMINE\HYDROCODONE:Abdominal discomfort:0.65), (CLOMIPRAMINE HYDROCHLORIDE:Abdominal discomfort:0.19), (COLESTIPOL:Abdominal discomfort:0.46), (CORTISONE ACETATE:Abdominal discomfort:0.27), (CYPROHEPTADINE HYDROCHLORIDE:Abdominal discomfort:0.23), (DABRAFENIB MESYLATE:Abdominal discomfort:0.26), (DACLATASVIR:Abdominal discomfort:0.47), (DACLIZUMAB:Abdominal discomfort:1.56), (DANAZOL:Abdominal discomfort:1.23), (DAPAGLIFLOZIN PROPANEDIOL\METFORMIN HYDROCHLORIDE:Abdominal discomfort:0.2), (DARATUMUMAB:Abdominal discomfort:0.09), (DASABUVIR:Abdominal discomfort:0.13), (DEXAMETHASONE ACETATE:Abdominal discomfort:1.17), (DIFLUNISAL:Abdominal discomfort:0.05), (DIGITOXIN:Abdominal discomfort:0.64), (DIPYRIDAMOLE:Abdominal discomfort:1.24), (DIPYRONE\PROMETHAZINE HYDROCHLORIDE:Abdominal discomfort:0.05), (DOLASETRON MESYLATE:Abdominal discomfort:0.7), (DORZOLAMIDE:Abdominal discomfort:0.96), (DUTASTERIDE\TAMSULOSIN HYDROCHLORIDE:Abdominal discomfort:0.45), (ECONAZOLE:Abdominal discomfort:0.66), (EFINACONAZOLE:Abdominal discomfort:0.42), (ELBASVIR\GRAZOPREVIR:Abdominal discomfort:0.33), (ELOTUZUMAB:Abdominal discomfort:0.9), (ELTROMBOPAG OLAMINE:Abdominal discomfort:0.45), (ERIBULIN:Abdominal discomfort:0.49), (ERTAPENEM SODIUM:Abdominal discomfort:0.05), (ESTRIOL:Abdominal discomfort:0.18), (ESTRONE\SUS SCROFA OVARY:Abdominal discomfort:1.13), (ETHACRYNIC ACID:Abdominal discomfort:0.24), (ETHINYL ESTRADIOL\NORGESTREL:Abdominal discomfort:0.31), (ETRETINATE:Abdominal discomfort:0.6), (FENOTEROL HYDROBROMIDE\IPRATROPIUM BROMIDE:Abdominal discomfort:0.33), (FERROUS BISGLYCINATE:Abdominal discomfort:0.1), (FERROUS SULFATE ANHYDROUS:Abdominal discomfort:0.21), (FERROUS SULFATE\GLYCINE:Abdominal discomfort:0.1), (FLUINDIONE:Abdominal discomfort:0.61), (FONDAPARINUX:Abdominal discomfort:0), (FONDAPARINUX SODIUM:Abdominal discomfort:0.38), (FORMOTEROL:Abdominal discomfort:0.85), (FOSINOPRIL SODIUM:Abdominal discomfort:0.06), (GENTAMICIN:Abdominal discomfort:0.35), (GIMERACIL\OTERACIL\TEGAFUR:Abdominal discomfort:1.14), (GRANULOCYTE COLONY-STIMULATING FACTOR NOS:Abdominal discomfort:0.25), (GUANFACINE HYDROCHLORIDE:Abdominal discomfort:0.59), (HEPARIN CALCIUM:Abdominal discomfort:0.07), (HYALURONATE SODIUM:Abdominal discomfort:0.76), (HYDROCHLOROTHIAZIDE\TELMISARTAN:Abdominal discomfort:1.21), (HYDROCODONE BITARTRATE\IBUPROFEN:Abdominal discomfort:0.97), (HYOSCYAMINE SULFATE\METHENAMINE\METHYLENE BLUE\PHENYL SALICYLATE\SODIUM PHOSPHATE, MONOBASIC, MONOHYDRATE:Abdominal discomfort:0.07), (IDELALISIB:Abdominal discomfort:0.64), (ILOPROST:Abdominal discomfort:1.94), (IMMUNE GLOBULIN NOS:Abdominal discomfort:0.33), (INTERFERON ALFA-2A:Abdominal discomfort:0.78), (INTERFERON ALFA-2B:Abdominal discomfort:1.05), (INTERFERON GAMMA-1B:Abdominal discomfort:0.06), (IODINE:Abdominal discomfort:0.8), (IRINOTECAN HYDROCHLORIDE:Abdominal discomfort:1.15), (IRON\POLYSACCHARIDES:Abdominal discomfort:0.15), (IVABRADINE HYDROCHLORIDE:Abdominal discomfort:0.15), (LANREOTIDE:Abdominal discomfort:0.14), (LENVATINIB:Abdominal discomfort:0.31), (LEVALBUTEROL:Abdominal discomfort:0.42), (LEVODOPA:Abdominal discomfort:0.16), (LEVOLEUCOVORIN CALCIUM:Abdominal discomfort:0.54), (LUTETIUM LU-177:Abdominal discomfort:0.2), (MEDROXYPROGESTERONE:Abdominal discomfort:0.32), (MEGESTROL:Abdominal discomfort:0.47), (MEPOLIZUMAB:Abdominal discomfort:0.67), (METFORMIN HYDROCHLORIDE\ROSIGLITAZONE MALEATE:Abdominal discomfort:0.38), (METHENAMINE MANDELATE:Abdominal discomfort:0.04), (MIDODRINE HYDROCHLORIDE:Abdominal discomfort:0.94), (MOEXIPRIL HYDROCHLORIDE:Abdominal discomfort:0.5), (MOXONIDINE:Abdominal discomfort:0.4), (MUPIROCIN\MUPIROCIN CALCIUM:Abdominal discomfort:1.44), (NADROPARIN CALCIUM:Abdominal discomfort:0.5), (NALOXONE HYDROCHLORIDE\TILIDINE HYDROCHLORIDE:Abdominal discomfort:0.69), (NEPAFENAC:Abdominal discomfort:0.13), (NIRAPARIB:Abdominal discomfort:0.92), (NOREPINEPHRINE:Abdominal discomfort:0.02), (NORETHINDRONE:Abdominal discomfort:0.9), (OLAPARIB:Abdominal discomfort:0.83), (OXAPROZIN:Abdominal discomfort:0.22), (OXYBUTYNIN\OXYBUTYNIN CHLORIDE:Abdominal discomfort:0.53), (PARATHYROID HORMONE:Abdominal discomfort:0.09), (PASIREOTIDE:Abdominal discomfort:0.24), (PEGINTERFERON BETA-1A:Abdominal discomfort:1.37), (PILSICAINIDE HYDROCHLORIDE:Abdominal discomfort:0.2), (PRAMLINTIDE ACETATE:Abdominal discomfort:0.03), (PRAMOXINE HYDROCHLORIDE:Abdominal discomfort:0.04), (PREDNISOLONE SODIUM PHOSPHATE:Abdominal discomfort:0.56), (PROTRIPTYLINE HYDROCHLORIDE:Abdominal discomfort:0.55), (PYRAZINAMIDE:Abdominal discomfort:0.75), (QUINACRINE:Abdominal discomfort:0.26), (RADIUM RA-223 DICHLORIDE:Abdominal discomfort:0.25), (RAMUCIRUMAB:Abdominal discomfort:0.67), (REGORAFENIB:Abdominal discomfort:1.57), (SALMETEROL:Abdominal discomfort:0.31), (SARPOGRELATE:Abdominal discomfort:0.25), (SELEGILINE HYDROCHLORIDE:Abdominal discomfort:0.03), (SIPULEUCEL-T:Abdominal discomfort:0.35), (SOFOSBUVIR\VELPATASVIR:Abdominal discomfort:0.2), (TAFLUPROST:Abdominal discomfort:0.34), (TASIMELTEON:Abdominal discomfort:0.05), (TEDUGLUTIDE\WATER:Abdominal discomfort:0.44), (TELOTRISTAT ETHYL:Abdominal discomfort:0.52), (TESTOSTERONE UNDECANOATE:Abdominal discomfort:0.35), (TETRABENAZINE:Abdominal discomfort:0.08), (TETRACYCLINE:Abdominal discomfort:1.84), (TINZAPARIN:Abdominal discomfort:0.14), (TRAMETINIB DIMETHYL SULFOXIDE:Abdominal discomfort:0.22), (TRIPTORELIN PAMOATE:Abdominal discomfort:0.25), (VARDENAFIL HYDROCHLORIDE TRIHYDRATE:Abdominal discomfort:0.29), (VILAZODONE HYDROCHLORIDE:Abdominal discomfort:1.2), (VISMODEGIB:Abdominal discomfort:0.21), (WARFARIN POTASSIUM:Abdominal discomfort:0.38), (ZOLMITRIPTAN:Abdominal discomfort:0.64), (.ALPHA.-TOCOPHEROL:Abdominal distension:0.49), (ACETAMINOPHEN\BUTALBITAL\CAFFEINE:Abdominal distension:0.89), (ACETAMINOPHEN\OXYCODONE\OXYCODONE HYDROCHLORIDE:Abdominal distension:0.07), (AMISULPRIDE:Abdominal distension:0.12), (BENAZEPRIL\BENAZEPRIL HYDROCHLORIDE:Abdominal distension:0.15), (BRINZOLAMIDE:Abdominal distension:0.21), (BUPRENORPHINE HYDROCHLORIDE:Abdominal distension:0.53), (CALCITONIN SALMON:Abdominal distension:0.05), (CALCITRIOL:Abdominal distension:0.94), (CALCIUM CARBONATE\VITAMIN D:Abdominal distension:0.37), (CANDESARTAN CILEXETIL\HYDROCHLOROTHIAZIDE:Abdominal distension:0.74), (CARVEDILOL PHOSPHATE:Abdominal distension:0.47), (CEFOTAXIME SODIUM:Abdominal distension:0.08), (CERTOLIZUMAB PEGOL:Abdominal distension:1.47), (CHOLESTYRAMINE:Abdominal distension:1.21), (CLINDAMYCIN:Abdominal distension:0.61), (CLOTRIMAZOLE:Abdominal distension:0.57), (DACLIZUMAB:Abdominal distension:0.16), (DANAZOL:Abdominal distension:0.03), (DASATINIB:Abdominal distension:1.25), (DESVENLAFAXINE SUCCINATE:Abdominal distension:0.29), (DEVICE:Abdominal distension:0.1), (DOFETILIDE:Abdominal distension:0.19), (EDOXABAN TOSYLATE:Abdominal distension:0.17), (ELETRIPTAN HYDROBROMIDE:Abdominal distension:0.47), (EPIRUBICIN:Abdominal distension:0.01), (EPLERENONE:Abdominal distension:0.05), (ESOMEPRAZOLE MAGNESIUM\NAPROXEN:Abdominal distension:0.02), (ESTROGENS, CONJUGATED\MEDROXYPROGESTERONE ACETATE:Abdominal distension:0.41), (EVOLOCUMAB:Abdominal distension:0.58), (EZETIMIBE\SIMVASTATIN:Abdominal distension:0.47), (FINGOLIMOD HYDROCHLORIDE:Abdominal distension:0.52), (FLUTICASONE:Abdominal distension:0.54), (FLUTICASONE FUROATE:Abdominal distension:0.21), (FORMOTEROL FUMARATE:Abdominal distension:0.06), (FORMOTEROL FUMARATE DIHYDRATE\MOMETASONE FUROATE:Abdominal distension:0.16), (FULVESTRANT:Abdominal distension:0.93), (GLYCOPYRRONIUM\INDACATEROL:Abdominal distension:0.07), (HERBALS\TURMERIC:Abdominal distension:0.13), (HYDROXYZINE PAMOATE:Abdominal distension:0.92), (IBRUTINIB:Abdominal distension:1.16), (IMATINIB MESYLATE:Abdominal distension:0.94), (IMIPRAMINE HYDROCHLORIDE:Abdominal distension:0.23), (INDAPAMIDE\PERINDOPRIL:Abdominal distension:0.22), (INVESTIGATIONAL PRODUCT:Abdominal distension:0.04), (IPRATROPIUM:Abdominal distension:0.72), (IRINOTECAN HYDROCHLORIDE:Abdominal distension:0.41), (ISOSORBIDE MONONITRATE:Abdominal distension:1.46), (ITRACONAZOLE:Abdominal distension:0.1), (LERCANIDIPINE:Abdominal distension:0.46), (LEVOLEUCOVORIN CALCIUM:Abdominal distension:0.21), (LINAGLIPTIN:Abdominal distension:0.64), (LISDEXAMFETAMINE DIMESYLATE:Abdominal distension:0.54), (METHENAMINE HIPPURATE:Abdominal distension:0.16), (METHYLPREDNISOLONE ACETATE:Abdominal distension:0.69), (MYCOPHENOLATE MOFETIL\MYCOPHENOLATE MOFETIL HYDROCHLORIDE:Abdominal distension:0.12), (NABUMETONE:Abdominal distension:0.14), (NEBIVOLOL:Abdominal distension:0.44), (NEVIRAPINE:Abdominal distension:0.35), (NILOTINIB:Abdominal distension:1.19), (NINTEDANIB:Abdominal distension:0.32), (NITROFURANTOIN\NITROFURANTOIN MONOHYDRATE:Abdominal distension:0.13), (OLANZAPINE:Abdominal distension:1.31), (OLAPARIB:Abdominal distension:0.05), (OLODATEROL HYDROCHLORIDE\TIOTROPIUM BROMIDE MONOHYDRATE:Abdominal distension:0.21), (PANTOPRAZOLE MAGNESIUM:Abdominal distension:0.68), (PAZOPANIB HYDROCHLORIDE:Abdominal distension:0.3), (PHENYTOIN:Abdominal distension:0.31), (PIROXICAM:Abdominal distension:0.01), (PITAVASTATIN CALCIUM:Abdominal distension:0.38), (POSACONAZOLE:Abdominal distension:0.38), (PREDNISOLONE ACETATE:Abdominal distension:0.21), (PRIMIDONE:Abdominal distension:0.77), (RALOXIFENE HYDROCHLORIDE:Abdominal distension:0.09), (RANOLAZINE:Abdominal distension:0.7), (REGORAFENIB:Abdominal distension:0.43), (RIZATRIPTAN BENZOATE:Abdominal distension:0.55), (SCOPOLAMINE:Abdominal distension:0.38), (SECUKINUMAB:Abdominal distension:0.51), (SOTALOL:Abdominal distension:0.59), (SUMATRIPTAN:Abdominal distension:0.16), (SUMATRIPTAN\SUMATRIPTAN SUCCINATE:Abdominal distension:0.83), (TERIFLUNOMIDE:Abdominal distension:0.65), (TIMOLOL:Abdominal distension:0.61), (TIOTROPIUM:Abdominal distension:0.25), (VALGANCICLOVIR HYDROCHLORIDE:Abdominal distension:0.16), (VARENICLINE TARTRATE:Abdominal distension:1.17), (.ALPHA.-TOCOPHEROL\VITAMINS:Abdominal pain:0.01), (ACETAMINOPHEN\CAFFEINE:Abdominal pain:0.28), (ACETAMINOPHEN\PROPOXYPHENE NAPSYLATE:Abdominal pain:0.78), (ACETAZOLAMIDE:Abdominal pain:1.37), (ACITRETIN:Abdominal pain:0.22), (ACTIVATED CHARCOAL:Abdominal pain:0.37), (AGALSIDASE BETA:Abdominal pain:0.82), (ALBUTEROL\IPRATROPIUM:Abdominal pain:0.79), (ALDESLEUKIN:Abdominal pain:0.43), (ALISKIREN HEMIFUMARATE:Abdominal pain:0.8), (ALUMINUM HYDROXIDE\MAGNESIUM OXIDE\OXETHAZAINE:Abdominal pain:0.15), (AMANTADINE:Abdominal pain:0.75), (AMILORIDE HYDROCHLORIDE\HYDROCHLOROTHIAZIDE:Abdominal pain:1.1), (AMLODIPINE BESYLATE\BENAZEPRIL HYDROCHLORIDE:Abdominal pain:0.16), (AMLODIPINE BESYLATE\HYDROCHLOROTHIAZIDE\OLMESARTAN MEDOXOMIL:Abdominal pain:0.82), (AMLODIPINE BESYLATE\HYDROCHLOROTHIAZIDE\VALSARTAN:Abdominal pain:0.73), (AMLODIPINE BESYLATE\TELMISARTAN:Abdominal pain:0.28), (AMLODIPINE\VALSARTAN:Abdominal pain:0.93), (AMMONIUM CHLORIDE\DEXTROMETHORPHAN\SODIUM CITRATE:Abdominal pain:0), (AMOXICILLIN\CLARITHROMYCIN\ESOMEPRAZOLE:Abdominal pain:0.62), (ANTIHISTAMINES NOS:Abdominal pain:0.52), (ASPARAGINASE:Abdominal pain:0.41), (ASPIRIN\CLOPIDOGREL:Abdominal pain:0.31), (ASPIRIN\MAGNESIUM OXIDE:Abdominal pain:0.44), (ATRACURIUM:Abdominal pain:0.42), (ATROPINE SULFATE:Abdominal pain:0.62), (AZELASTINE:Abdominal pain:0.43), (AZILSARTAN KAMEDOXOMIL:Abdominal pain:0.97), (AZITHROMYCIN OR METHOTREXATE SODIUM:Abdominal pain:0.16), (AZOSEMIDE:Abdominal pain:0.22), (BALSALAZIDE DISODIUM:Abdominal pain:0.85), (BECLOMETHASONE DIPROPIONATE\FORMOTEROL:Abdominal pain:0), (BECLOMETHASONE\FORMOTEROL:Abdominal pain:0.17), (BENZTROPINE:Abdominal pain:0.09), (BETAMETHASONE DIPROPIONATE\CALCIPOTRIENE:Abdominal pain:0.13), (BETAMETHASONE DIPROPIONATE\CALCIPOTRIENE\CALCIPOTRIENE HYDRATE:Abdominal pain:0.32), (BETAMETHASONE DIPROPIONATE\CLOTRIMAZOLE:Abdominal pain:0.19), (BETAMETHASONE\CALCIPOTRIENE:Abdominal pain:0.62), (BETHANECHOL CHLORIDE:Abdominal pain:0.22), (BOCEPREVIR:Abdominal pain:0.22), (BOTULINUM TOXIN TYPE A:Abdominal pain:0.29), (BRIMONIDINE TARTRATE\TIMOLOL MALEATE:Abdominal pain:0.12), (BUDESONIDE\FORMOTEROL FUMARATE:Abdominal pain:1.15), (BUPRENORPHINE HYDROCHLORIDE\NALOXONE HYDROCHLORIDE:Abdominal pain:0.59), (CABAZITAXEL:Abdominal pain:0.18), (CABOZANTINIB:Abdominal pain:0.12), (CABOZANTINIB S-MALATE:Abdominal pain:0.31), (CALCIPOTRIENE:Abdominal pain:0.43), (CANAGLIFLOZIN\METFORMIN HYDROCHLORIDE:Abdominal pain:1.38), (CARBOXYMETHYLCELLULOSE SODIUM:Abdominal pain:0.02), (CARVEDILOL PHOSPHATE:Abdominal pain:1.64), (CEFOTAXIME\CEFOTAXIME SODIUM:Abdominal pain:0.8), (CEFPROZIL:Abdominal pain:0.28), (CERTOPARIN SODIUM:Abdominal pain:0.21), (CETIRIZINE HYDROCHLORIDE\PSEUDOEPHEDRINE HYDROCHLORIDE:Abdominal pain:0.42), (CEVIMELINE HYDROCHLORIDE:Abdominal pain:0.03), (CHLOROTHIAZIDE:Abdominal pain:0.39), (CIPROFIBRATE:Abdominal pain:0.05), (CLOBAZAM:Abdominal pain:0.45), (COBICISTAT\ELVITEGRAVIR\EMTRICITABINE\TENOFOVIR ALAFENAMIDE FUMARATE:Abdominal pain:0.26), (COD LIVER OIL:Abdominal pain:0.35), (COLISTIMETHATE SODIUM:Abdominal pain:0.58), (COLISTIN:Abdominal pain:0.58), (CYAMEMAZINE:Abdominal pain:0.04), (DABRAFENIB MESYLATE:Abdominal pain:0.44), (DACLATASVIR:Abdominal pain:0.3), (DACLIZUMAB:Abdominal pain:1.49), (DANAZOL:Abdominal pain:1.16), (DANTROLENE SODIUM:Abdominal pain:0.29), (DAPSONE:Abdominal pain:1.38), (DARATUMUMAB:Abdominal pain:0.63), (DESFLURANE:Abdominal pain:0.78), (DESMOPRESSIN:Abdominal pain:0.21), (DESONIDE:Abdominal pain:0.76), (DEXMEDETOMIDINE HYDROCHLORIDE:Abdominal pain:0.74), (DOBUTAMINE HYDROCHLORIDE:Abdominal pain:0.4), (DOFETILIDE:Abdominal pain:1.14), (DOLASETRON MESYLATE:Abdominal pain:0.7), (DONEPEZIL HYDROCHLORIDE:Abdominal pain:0.09), (DORZOLAMIDE:Abdominal pain:0.57), (DORZOLAMIDE HYDROCHLORIDE:Abdominal pain:0.12), (DOXEPIN HYDROCHLORIDE:Abdominal pain:0.72), (EMPAGLIFLOZIN\LINAGLIPTIN:Abdominal pain:0.81), (ENALAPRIL MALEATE\HYDROCHLOROTHIAZIDE:Abdominal pain:0.06), (EPHEDRINE:Abdominal pain:0.09), (EPHEDRINE HYDROCHLORIDE:Abdominal pain:0.03), (EPIRUBICIN HYDROCHLORIDE:Abdominal pain:0.17), (ERIBULIN:Abdominal pain:0.97), (ERIBULIN MESYLATE:Abdominal pain:0.27), (ESTROGENS, CONJUGATED\MEDROXYPROGESTERONE ACETATE:Abdominal pain:0.75), (ETHACRYNIC ACID:Abdominal pain:0.21), (ETHINYL ESTRADIOL\GESTODENE:Abdominal pain:0.18), (ETRETINATE:Abdominal pain:0.18), (EVOLOCUMAB:Abdominal pain:1.14), (FERRIC CARBOXYMALTOSE:Abdominal pain:0.04), (FERROUS SULFATE\GLYCINE:Abdominal pain:0.65), (FLUDROCORTISONE ACETATE:Abdominal pain:1.1), (FLUOROMETHOLONE:Abdominal pain:0.3), (FLUOROURACIL\IRINOTECAN\LEUCOVORIN:Abdominal pain:0.37), (FLUVASTATIN SODIUM:Abdominal pain:0.59), (FORMOTEROL FUMARATE:Abdominal pain:1.6), (FORMOTEROL FUMARATE\MOMETASONE FUROATE:Abdominal pain:0.95), (FOSFOMYCIN:Abdominal pain:0.6), (FOSPHENYTOIN\FOSPHENYTOIN SODIUM:Abdominal pain:0.41), (GEFITINIB:Abdominal pain:0.51), (GLUCAGON:Abdominal pain:0.26), (GLYCOPYRROLATE\INDACATEROL MALEATE:Abdominal pain:0.19), (GOSERELIN ACETATE:Abdominal pain:0.2), (GUANFACINE:Abdominal pain:0.11), (HYDROCHLOROTHIAZIDE\LOSARTAN:Abdominal pain:0.34), (HYDROXYCHLOROQUINE DIPHOSPHATE:Abdominal pain:0.67), (IBRITUMOMAB TIUXETAN:Abdominal pain:0.52), (ILOPROST:Abdominal pain:1.09), (IMATINIB MESYLATE:Abdominal pain:2.28), (IMMUNE GLOBULIN NOS:Abdominal pain:0.08), (INTERFERON ALFA-2A:Abdominal pain:0.87), (INTERFERON ALFA-2B:Abdominal pain:1.09), (INTERFERON GAMMA-1B:Abdominal pain:0.6), (IRON\POLYSACCHARIDES:Abdominal pain:0.06), (ISOFLURANE:Abdominal pain:0.63), (IVABRADINE:Abdominal pain:0.14), (LECITHIN:Abdominal pain:0.12), (LIOTHYRONINE:Abdominal pain:0.43), (LUMACAFTOR:Abdominal pain:0.21), (LUTETIUM LU-177:Abdominal pain:0.17), (MAGNESIUM ASPARTATE\POTASSIUM ASPARTATE:Abdominal pain:0.12), (MEDROXYPROGESTERONE:Abdominal pain:0.32), (MEPOLIZUMAB:Abdominal pain:0.7), (METFORMIN HYDROCHLORIDE\SAXAGLIPTIN HYDROCHLORIDE:Abdominal pain:0.03), (METHENOLONE ENANTHATE:Abdominal pain:0.16), (METHYLENE BLUE:Abdominal pain:0.21), (MEXILETINE:Abdominal pain:0.06), (MIDODRINE HYDROCHLORIDE:Abdominal pain:0.64), (MOEXIPRIL HYDROCHLORIDE:Abdominal pain:0.33), (MOXONIDINE:Abdominal pain:0.88), (NATEGLINIDE:Abdominal pain:0.76), (NEOMYCIN:Abdominal pain:0.37), (ONABOTULINUMTOXINA:Abdominal pain:0.38), (ORPHENADRINE:Abdominal pain:0.22), (PARICALCITOL:Abdominal pain:0.28), (PATIROMER:Abdominal pain:0.36), (PEGINTERFERON BETA-1A:Abdominal pain:0.84), (PENTOSTATIN:Abdominal pain:0.88), (PIMECROLIMUS:Abdominal pain:0.24), (PIRFENIDONE:Abdominal pain:0.17), (PORFIMER SODIUM:Abdominal pain:0.66), (PREDNISOLONE SODIUM PHOSPHATE:Abdominal pain:0.9), (PRISTINAMYCIN:Abdominal pain:0.5), (PYRIDOSTIGMINE BROMIDE:Abdominal pain:0.6), (PYRIMETHAMINE:Abdominal pain:0.14), (QUINACRINE:Abdominal pain:0.18), (QUINAPRIL:Abdominal pain:0.43), (RADIATION THERAPY:Abdominal pain:1.21), (RETINOL:Abdominal pain:1.51), (RIVASTIGMINE:Abdominal pain:0.49), (RIZATRIPTAN:Abdominal pain:0.38), (ROFLUMILAST:Abdominal pain:1.83), (RUCAPARIB CAMSYLATE:Abdominal pain:0.05), (SACUBITRIL\VALSARTAN:Abdominal pain:0.54), (SALMETEROL XINAFOATE:Abdominal pain:0.37), (SELEXIPAG:Abdominal pain:0.72), (SIPULEUCEL-T:Abdominal pain:0.29), (SODIUM CITRATE:Abdominal pain:0.25), (SOFOSBUVIR\VELPATASVIR:Abdominal pain:0.13), (SORBITOL:Abdominal pain:0.1), (SOTALOL HYDROCHLORIDE:Abdominal pain:0.33), (SPIRAMYCIN:Abdominal pain:0.19), (SUFENTANIL:Abdominal pain:0.99), (SUFENTANIL CITRATE:Abdominal pain:0.79), (SULINDAC:Abdominal pain:0.21), (TAZOBACTAM:Abdominal pain:0.88), (TELOTRISTAT ETHYL:Abdominal pain:0.83), (TESTOSTERONE UNDECANOATE:Abdominal pain:0.59), (THIOPENTAL:Abdominal pain:0.33), (THYROID, PORCINE:Abdominal pain:0.83), (THYROID, PORCINE\THYROID, UNSPECIFIED:Abdominal pain:0.35), (TICLOPIDINE:Abdominal pain:0.18), (TIGECYCLINE:Abdominal pain:0.9), (TRAMETINIB DIMETHYL SULFOXIDE:Abdominal pain:0.43), (TRANDOLAPRIL:Abdominal pain:0.41), (TREPROSTINIL DIOLAMINE:Abdominal pain:0.18), (TRICHLORMETHIAZIDE:Abdominal pain:0.1), (TRIHEXYPHENIDYL:Abdominal pain:0), (TRIMIPRAMINE MALEATE:Abdominal pain:0.44), (UMECLIDINIUM BROMIDE:Abdominal pain:0.61), (UMECLIDINIUM BROMIDE\VILANTEROL TRIFENATATE:Abdominal pain:1.11), (VOGLIBOSE:Abdominal pain:0.1), (YEAST:Abdominal pain:0.12), (ZAFIRLUKAST:Abdominal pain:0.58), (ZILEUTON:Abdominal pain:0.23), (ZINC:Abdominal pain:0.94), (.ALPHA.1-PROTEINASE INHIBITOR HUMAN:Abdominal pain upper:1.17), (ACETAMINOPHEN\PROPOXYPHENE NAPSYLATE:Abdominal pain upper:0.64), (ACITRETIN:Abdominal pain upper:0.38), (AGALSIDASE BETA:Abdominal pain upper:0.73), (ALDESLEUKIN:Abdominal pain upper:0.35), (ALISKIREN HEMIFUMARATE:Abdominal pain upper:0.56), (ALOGLIPTIN BENZOATE:Abdominal pain upper:0.38), (AMANTADINE:Abdominal pain upper:0.68), (AMINO ACIDS\DEXTROSE\ELECTROLYTES NOS:Abdominal pain upper:0.72), (AMLODIPINE BESYLATE\ATORVASTATIN CALCIUM:Abdominal pain upper:0.69), (AMLODIPINE BESYLATE\HYDROCHLOROTHIAZIDE\OLMESARTAN MEDOXOMIL:Abdominal pain upper:0.83), (AMLODIPINE BESYLATE\HYDROCHLOROTHIAZIDE\VALSARTAN:Abdominal pain upper:0.8), (AMOXICILLIN\CLARITHROMYCIN\ESOMEPRAZOLE:Abdominal pain upper:0.27), (AMPICILLIN SODIUM\SULBACTAM SODIUM:Abdominal pain upper:0.82), (ANAGRELIDE HYDROCHLORIDE:Abdominal pain upper:0.06), (ANTIHISTAMINES NOS:Abdominal pain upper:0.18), (ASPIRIN\DIPYRIDAMOLE:Abdominal pain upper:0.04), (ASPIRIN\MAGNESIUM OXIDE:Abdominal pain upper:0.46), (ATOVAQUONE:Abdominal pain upper:0.71), (ATROPINE\DIPHENOXYLATE:Abdominal pain upper:0.5), (AZITHROMYCIN OR METHOTREXATE SODIUM:Abdominal pain upper:0.61), (BACITRACIN:Abdominal pain upper:0.14), (BECLOMETHASONE:Abdominal pain upper:0.26), (BENZTROPINE:Abdominal pain upper:0.12), (BENZTROPINE MESYLATE:Abdominal pain upper:0.04), (BETAMETHASONE DIPROPIONATE\CALCIPOTRIENE:Abdominal pain upper:0.29), (BETAMETHASONE DIPROPIONATE\CALCIPOTRIENE\CALCIPOTRIENE HYDRATE:Abdominal pain upper:0.46), (BETAMETHASONE DIPROPIONATE\CLOTRIMAZOLE:Abdominal pain upper:0.15), (BICALUTAMIDE:Abdominal pain upper:0.4), (BISOPROLOL FUMARATE\HYDROCHLOROTHIAZIDE:Abdominal pain upper:0.09), (BLINATUMOMAB:Abdominal pain upper:0.95), (BUDESONIDE\FORMOTEROL:Abdominal pain upper:0.09), (CABAZITAXEL:Abdominal pain upper:0.26), (CALCITONIN:Abdominal pain upper:0.82), (CALCITONIN SALMON:Abdominal pain upper:1.27), (CALCIUM ACETATE:Abdominal pain upper:1.74), (CALCIUM CARBONATE\ERGOCALCIFEROL\RETINOL:Abdominal pain upper:0.17), (CALCIUM LACTATE:Abdominal pain upper:0.36), (CALCIUM POLYCARBOPHIL:Abdominal pain upper:0.42), (CALCIUM\MAGNESIUM:Abdominal pain upper:0.48), (CALCIUM\MAGNESIUM\ZINC:Abdominal pain upper:0.05), (CANAGLIFLOZIN\METFORMIN HYDROCHLORIDE:Abdominal pain upper:1.1), (CARVEDILOL PHOSPHATE:Abdominal pain upper:1.43), (CASANTHRANOL\DOCUSATE SODIUM:Abdominal pain upper:0.55), (CEFOTAXIME\CEFOTAXIME SODIUM:Abdominal pain upper:0.5), (CEFUROXIME AXETIL:Abdominal pain upper:0.47), (CELIPROLOL:Abdominal pain upper:0.25), (CERTOPARIN SODIUM:Abdominal pain upper:0), (CHLOROTHIAZIDE:Abdominal pain upper:0.09), (CLOMIPRAMINE HYDROCHLORIDE:Abdominal pain upper:0.59), (CODEINE PHOSPHATE\GUAIFENESIN:Abdominal pain upper:0.31), (COLCHICINE\DICYCLOMINE HYDROCHLORIDE:Abdominal pain upper:0), (CYPROHEPTADINE HYDROCHLORIDE:Abdominal pain upper:0.45), (DABIGATRAN:Abdominal pain upper:1), (DABRAFENIB MESYLATE:Abdominal pain upper:0.38), (DACLIZUMAB:Abdominal pain upper:1.22), (DANTROLENE SODIUM:Abdominal pain upper:0.07), (DAPSONE:Abdominal pain upper:0.41), (DASABUVIR:Abdominal pain upper:0.26), (DEGARELIX:Abdominal pain upper:0.13), (DESFLURANE:Abdominal pain upper:0.48), (DESMOPRESSIN:Abdominal pain upper:0.05), (DIGITOXIN:Abdominal pain upper:0.92), (DIPHENHYDRAMINE HYDROCHLORIDE\LIDOCAINE HYDROCHLORIDE\NYSTATIN:Abdominal pain upper:0.08), (DOBUTAMINE HYDROCHLORIDE:Abdominal pain upper:0.27), (DOFETILIDE:Abdominal pain upper:1.23), (DOLASETRON MESYLATE:Abdominal pain upper:0.43), (DORZOLAMIDE:Abdominal pain upper:0.23), (DORZOLAMIDE HYDROCHLORIDE:Abdominal pain upper:0.17), (DOXYLAMINE SUCCINATE:Abdominal pain upper:0.41), (DUTASTERIDE\TAMSULOSIN HYDROCHLORIDE:Abdominal pain upper:0.51), (ELBASVIR\GRAZOPREVIR:Abdominal pain upper:0.15), (EMPAGLIFLOZIN\LINAGLIPTIN:Abdominal pain upper:0.7), (ENALAPRIL MALEATE\HYDROCHLOROTHIAZIDE:Abdominal pain upper:0.24), (ERIBULIN:Abdominal pain upper:0.74), (ERIBULIN MESYLATE:Abdominal pain upper:0.08), (ERTAPENEM SODIUM:Abdominal pain upper:0.1), (ESMOLOL HYDROCHLORIDE:Abdominal pain upper:0.28), (ESTROGENS, CONJUGATED\MEDROXYPROGESTERONE ACETATE:Abdominal pain upper:1.15), (ETRETINATE:Abdominal pain upper:0.7), (EVOLOCUMAB:Abdominal pain upper:1.47), (FENOTEROL HYDROBROMIDE\IPRATROPIUM BROMIDE:Abdominal pain upper:0.65), (FERROUS SULFATE\GLYCINE:Abdominal pain upper:0.36), (FISH OIL\OMEGA-3 FATTY ACIDS:Abdominal pain upper:0.36), (FLAVIN ADENINE DINUCLEOTIDE:Abdominal pain upper:0), (FLUDROCORTISONE:Abdominal pain upper:0.31), (FLUTICASONE FUROATE:Abdominal pain upper:1.26), (FONDAPARINUX:Abdominal pain upper:0.24), (FORMOTEROL FUMARATE:Abdominal pain upper:1.67), (FORMOTEROL FUMARATE\MOMETASONE FUROATE:Abdominal pain upper:1.07), (FOSPHENYTOIN\FOSPHENYTOIN SODIUM:Abdominal pain upper:0.19), (FUSIDIC ACID:Abdominal pain upper:1.45), (GEMCITABINE HYDROCHLORIDE:Abdominal pain upper:0.86), (GLYCOPYRROLATE\INDACATEROL MALEATE:Abdominal pain upper:0.34), (GLYCOPYRRONIUM\INDACATEROL:Abdominal pain upper:1.7), (GLYCOSAMINOGLYCANS:Abdominal pain upper:0.38), (GRANULOCYTE COLONY-STIMULATING FACTOR NOS:Abdominal pain upper:0.57), (GUANFACINE:Abdominal pain upper:0.28), (HERBALS\TURMERIC:Abdominal pain upper:1.11), (HYDROCHLOROTHIAZIDE\LOSARTAN:Abdominal pain upper:0.15), (HYDROCODONE BITARTRATE\IBUPROFEN:Abdominal pain upper:0.59), (HYDROXYCHLOROQUINE DIPHOSPHATE:Abdominal pain upper:0.64), (IBRITUMOMAB TIUXETAN:Abdominal pain upper:1.08), (ILOPROST:Abdominal pain upper:1.19), (IMIPRAMINE HYDROCHLORIDE:Abdominal pain upper:0.92), (INFLIXIMAB-DYYB:Abdominal pain upper:0.94), (INTERFERON:Abdominal pain upper:0.59), (INTERFERON ALFA-2A:Abdominal pain upper:1.17), (INTERFERON ALFA-2B:Abdominal pain upper:1.02), (ISONIAZID\PYRAZINAMIDE\RIFAMPIN:Abdominal pain upper:0.33), (IVACAFTOR:Abdominal pain upper:0.28), (LABETALOL HYDROCHLORIDE:Abdominal pain upper:1.29), (LACTASE:Abdominal pain upper:0.68), (LENVATINIB:Abdominal pain upper:0.39), (LEVOTHYROXINE SODIUM\POTASSIUM IODIDE:Abdominal pain upper:0.24), (LOMITAPIDE MESYLATE:Abdominal pain upper:0.57), (LOMUSTINE:Abdominal pain upper:0.01), (LUTETIUM LU-177:Abdominal pain upper:0.11), (MAGNESIUM ASPARTATE\POTASSIUM ASPARTATE:Abdominal pain upper:0.15), (MEDROXYPROGESTERONE:Abdominal pain upper:0.02), (MELPERONE:Abdominal pain upper:0.34), (METFORMIN HYDROCHLORIDE\SAXAGLIPTIN HYDROCHLORIDE:Abdominal pain upper:0.08), (METHENOLONE ENANTHATE:Abdominal pain upper:0.16), (MIDODRINE:Abdominal pain upper:0.86), (MIDODRINE HYDROCHLORIDE:Abdominal pain upper:0.51), (MITOXANTRONE HYDROCHLORIDE:Abdominal pain upper:0.67), (MOEXIPRIL HYDROCHLORIDE:Abdominal pain upper:0.07), (NATEGLINIDE:Abdominal pain upper:0.71), (NICARDIPINE:Abdominal pain upper:1.19), (NITROFURANTOIN\NITROFURANTOIN MONOHYDRATE:Abdominal pain upper:0.56), (NOREPINEPHRINE BITARTRATE:Abdominal pain upper:0.54), (OLMESARTAN:Abdominal pain upper:0.71), (OLOPATADINE:Abdominal pain upper:0.4), (ORPHENADRINE:Abdominal pain upper:0.25), (OSELTAMIVIR:Abdominal pain upper:1.3), (OSIMERTINIB:Abdominal pain upper:0.46), (OXYBUTYNIN\OXYBUTYNIN CHLORIDE:Abdominal pain upper:0.06), (PARATHYROID HORMONE:Abdominal pain upper:0.01), (PATIROMER:Abdominal pain upper:0.32), (PENTOSTATIN:Abdominal pain upper:0.44), (PILSICAINIDE HYDROCHLORIDE:Abdominal pain upper:0.14), (PIMOBENDAN:Abdominal pain upper:0.18), (PIOGLITAZONE:Abdominal pain upper:0.49), (PIZOTYLINE:Abdominal pain upper:0.18), (PRAZOSIN HYDROCHLORIDE:Abdominal pain upper:0.48), (PREDNISOLONE SODIUM PHOSPHATE:Abdominal pain upper:0.59), (PRIMIDONE:Abdominal pain upper:2.01), (PYRIDOSTIGMINE BROMIDE:Abdominal pain upper:0.76), (PYRIMETHAMINE:Abdominal pain upper:0.11), (QUINACRINE:Abdominal pain upper:0.3), (RALOXIFENE HYDROCHLORIDE:Abdominal pain upper:1.56), (RILMENIDINE PHOSPHATE:Abdominal pain upper:0.61), (RILPIVIRINE HYDROCHLORIDE:Abdominal pain upper:0.03), (RIOCIGUAT:Abdominal pain upper:0.69), (ROCURONIUM BROMIDE:Abdominal pain upper:0.4), (SACUBITRIL\VALSARTAN:Abdominal pain upper:0.82), (SARPOGRELATE:Abdominal pain upper:0.61), (SIPULEUCEL-T:Abdominal pain upper:0.07), (SOFOSBUVIR\VELPATASVIR:Abdominal pain upper:0.06), (SPIRAMYCIN:Abdominal pain upper:0.24), (SUFENTANIL:Abdominal pain upper:0.05), (SUFENTANIL CITRATE:Abdominal pain upper:0.07), (TAMOXIFEN CITRATE:Abdominal pain upper:0.05), (TAZOBACTAM:Abdominal pain upper:0.64), (TEDUGLUTIDE\WATER:Abdominal pain upper:0.22), (TELOTRISTAT ETHYL:Abdominal pain upper:0.43), (TEMSIROLIMUS:Abdominal pain upper:0.22), (TERAZOSIN HYDROCHLORIDE:Abdominal pain upper:0.55), (TERBINAFINE:Abdominal pain upper:0.04), (THALIDOMIDE:Abdominal pain upper:2.27), (THEOPHYLLINE:Abdominal pain upper:0.16), (TIMOLOL MALEATE\TRAVOPROST:Abdominal pain upper:0.09), (TINZAPARIN:Abdominal pain upper:1.03), (TIOTROPIUM:Abdominal pain upper:1.37), (TOLTERODINE:Abdominal pain upper:0.34), (TRAMETINIB DIMETHYL SULFOXIDE:Abdominal pain upper:0.3), (TRICHLORMETHIAZIDE:Abdominal pain upper:0.62), (TRIHEXYPHENIDYL:Abdominal pain upper:0.1), (TRIPTORELIN PAMOATE:Abdominal pain upper:0.02), (UMECLIDINIUM BROMIDE:Abdominal pain upper:0.45), (VINORELBINE\VINORELBINE TARTRATE:Abdominal pain upper:0.25), (ZAFIRLUKAST:Abdominal pain upper:0.4), (ZILEUTON:Abdominal pain upper:0.18), (ZIPRASIDONE HYDROCHLORIDE\ZIPRASIDONE MESYLATE:Abdominal pain upper:0.16), (ZIV-AFLIBERCEPT:Abdominal pain upper:0.17), (AMANTADINE HYDROCHLORIDE:Abnormal behaviour:0.04), (AMIODARONE:Abnormal behaviour:0.07), (AMISULPRIDE:Abnormal behaviour:0.53), (ARIPIPRAZOLE LAUROXIL:Abnormal behaviour:0.25), (BRIVARACETAM:Abnormal behaviour:0.85), (BUMETANIDE:Abnormal behaviour:0.01), (BUPRENORPHINE HYDROCHLORIDE\NALOXONE HYDROCHLORIDE:Abnormal behaviour:0.23), (CALCITRIOL:Abnormal behaviour:0.01), (CALCIUM CARBONATE\CHOLECALCIFEROL:Abnormal behaviour:0.19), (CANDESARTAN CILEXETIL:Abnormal behaviour:0.1), (DILTIAZEM:Abnormal behaviour:0.05), (ENOXAPARIN:Abnormal behaviour:0.75), (ESZOPICLONE:Abnormal behaviour:0.08), (EVOLOCUMAB:Abnormal behaviour:0.06), (FERROUS SULFATE:Abnormal behaviour:0.12), (FLURAZEPAM\FLURAZEPAM HYDROCHLORIDE:Abnormal behaviour:0.08), (HYDROMORPHONE HYDROCHLORIDE:Abnormal behaviour:0), (METHYLPREDNISOLONE SODIUM SUCCINATE:Abnormal behaviour:0.3), (NEBIVOLOL:Abnormal behaviour:0.37), (OMEPRAZOLE MAGNESIUM:Abnormal behaviour:0.43), (PRAZOSIN:Abnormal behaviour:0.22), (PRIMIDONE:Abnormal behaviour:0.41), (ROPINIROLE HYDROCHLORIDE:Abnormal behaviour:0.19), (SOTALOL:Abnormal behaviour:0.06), (TIZANIDINE HYDROCHLORIDE:Abnormal behaviour:0.09), (VARENICLINE TARTRATE:Abnormal behaviour:0.75), (WARFARIN:Abnormal behaviour:0.65), (ALBUTEROL SULFATE\IPRATROPIUM BROMIDE:Abnormal faeces:0.61), (AMIODARONE HYDROCHLORIDE:Abnormal faeces:0.37), (AMITRIPTYLINE HYDROCHLORIDE:Abnormal faeces:0.01),(ARMODAFINIL:Abnormal faeces:0.4), (BENAZEPRIL HYDROCHLORIDE:Abnormal faeces:0.17), (BISOPROLOL FUMARATE:Abnormal faeces:0.47), (CANDESARTAN:Abnormal faeces:0.31), (CANDESARTAN CILEXETIL:Abnormal faeces:0.33), (CANDESARTAN CILEXETIL\HYDROCHLOROTHIAZIDE:Abnormal faeces:0.29), (CARFILZOMIB:Abnormal faeces:0.11), (CARVEDILOL PHOSPHATE:Abnormal faeces:0.65), (CERTOLIZUMAB PEGOL:Abnormal faeces:0.86), (CIPROFLOXACIN HYDROCHLORIDE:Abnormal faeces:0.31), (CLONIDINE:Abnormal faeces:0.85), (CORTISONE\HYDROCORTISONE:Abnormal faeces:0.29), (DASATINIB:Abnormal faeces:0.59), (DEVICE:Abnormal faeces:0.08), (DIPHENHYDRAMINE:Abnormal faeces:0.7), (DOXAZOSIN\DOXAZOSIN MESYLATE:Abnormal faeces:0.38), (DULOXETINE:Abnormal faeces:0.33), (DUTASTERIDE:Abnormal faeces:0.44), (ELETRIPTAN HYDROBROMIDE:Abnormal faeces:0.31), (ENOXAPARIN:Abnormal faeces:0.91), (ESZOPICLONE:Abnormal faeces:0.33), (EVEROLIMUS:Abnormal faeces:0.44), (EVOLOCUMAB:Abnormal faeces:0.76), (FLUTICASONE:Abnormal faeces:0.37), (FULVESTRANT:Abnormal faeces:0.23), (HEPARIN SODIUM:Abnormal faeces:0.45), (HYDROXYCHLOROQUINE SULFATE:Abnormal faeces:0.47), (HYOSCYAMINE:Abnormal faeces:0.38), (ILOPROST:Abnormal faeces:0.17), (INSULIN HUMAN:Abnormal faeces:0.3), (INSULIN LISPRO:Abnormal faeces:0.27), (IRBESARTAN:Abnormal faeces:0.51), (LABETALOL\LABETALOL HYDROCHLORIDE:Abnormal faeces:0.15), (LEDIPASVIR\SOFOSBUVIR:Abnormal faeces:0.08), (LERCANIDIPINE:Abnormal faeces:0.26), (METHENAMINE HIPPURATE:Abnormal faeces:0.08), (METRONIDAZOLE:Abnormal faeces:0.68), (MOXIFLOXACIN HYDROCHLORIDE:Abnormal faeces:0.41), (NEBIVOLOL HYDROCHLORIDE:Abnormal faeces:0.39), (NILOTINIB:Abnormal faeces:0.32), (OXCARBAZEPINE:Abnormal faeces:0.6), (PANTOPRAZOLE MAGNESIUM:Abnormal faeces:0.27), (PRAMIPEXOLE:Abnormal faeces:0.1), (RABEPRAZOLE SODIUM:Abnormal faeces:0.05), (RALOXIFENE HYDROCHLORIDE:Abnormal faeces:0.32), (RANOLAZINE:Abnormal faeces:1.37), (SACUBITRIL\VALSARTAN:Abnormal faeces:0.66), (THYROID, PORCINE:Abnormal faeces:0.17), (TOLTERODINE TARTRATE:Abnormal faeces:0.05), (UMECLIDINIUM BROMIDE\VILANTEROL TRIFENATATE:Abnormal faeces:0.41), (VARENICLINE TARTRATE:Abnormal faeces:0.68), (ACETAMINOPHEN\CODEINE PHOSPHATE:Abscess:0.14), (BUDESONIDE:Abscess:0.43), (CELECOXIB:Abscess:0.3), (CIT ALOPRAM HYDROBROMIDE:Abscess:0.56), (DEXLANSOPRAZOLE:Abscess:0.07), (EPINEPHRINE: Abscess:0.04), (FERROUS SULFATE:Abscess:0.15), (IBRUTINIB:Abscess:0.46), (MELOXICAM:Abscess:0.1), (NIFEDIPI NE:Abscess:0.03), (OLANZAPINE:Abscess:0.32), (OMEPRAZOLE MAGNESIUM:Abscess:0.19), (OXCARBAZEPINE:Abscess:0.15), (TOFACITINIB CITRATE:Abscess:0.03), (OMEPRAZOLE MAGNESIUM:Accidental exposure to product:0.24), (BISOPROLOL FUMARATE:Acidosis:0.5), (BUDESONIDE:Acidosis:0.88), (CALCIUM CARBONATE\CHOLECALCIFEROL:Acidosis:0.61), (CALCIUM\VITAMIN D:Acidosis:0.11), (CEPHALEXIN:Acidosis:0.3), (CERTOLIZUMAB PEGOL:Acidosis:0.87), (CLINDAMYCIN\CLINDAMYCIN PHOSPHATE:Acidosis:0.44), (CLONIDINE:Acidosis:0.33), (DILTIAZEM:Acidosis:0.51), (DIV ALPROEX SODIUM:Acidosis:0.46), (ENOXAPARIN:Acidosis:0.84), (ESZOPICLONE:Acidosis:0.22), (EV OLOCUMAB:Acidosis:0.4), (FENOFIBRATE:Acidosis:0.04), (GLIPIZIDE:Acidosis:0.23), (LA BETALOL\LABETALOL HYDROCHLORIDE:Acidosis:0.12), (MELATONIN:Acidosis:0.05), (METRONIDAZOLE\MET RONIDAZOLE HYDROCHLORIDE:Acidosis:0.01), (NAPROXEN SODIUM:Acidosis:0.05), (NEBIVOLOL:Acidosis:0.38), (NILOTINIB:Acidosis:0.17), (OXCAR BAZEPINE:Acidosis:0.84), (OXYGEN:Acidosis:0.33), (POLYETHYLENE GLYCOLS:Acidosis:0.27), (PRIMIDONE:Acidosis:0.08), (RANOLAZINE:Acidosis:0.2), (RISE DRONATE SODIUM:Acidosis:0.19), (TERIFLUNOMIDE:Acidosis:0.2), (TIOTROPIUM BROMIDE MONOHYDRATE:Acidosis:0.16), (.ALPHA.1-PROTEINASE INHIBITOR HUMAN:Acne:0.65), (ACETAMINOPHEN\OXYCODONE\OXYCODONE HYDROCHLORIDE:Acne:0.83), (ACETAMINOPHEN\PROPOXYPHENE NAPSYLATE:Acne:0.86), (ACETAMINOPHEN\TRAMADOL:Acne:0.06), (ADO-TRASTUZUMAB EMTANSINE:Acne:0.09), (AMANTADINE:Acne:0.09), (AMINO ACIDS\DEXTROSE\ELECTROLYTES NOS:Acne:0.07), (AMIODARONE HYDROCHLORIDE:Acne:0.33), (AMMONIUM LACTATE:Acne:0.33), (APREPITANT:Acne:0.1), (AZELASTINE HYDROCHLORIDE\FLUTICASONE PROPIONATE:Acne:0.13), (BACITRACIN:Acne:0.17), (BEVACIZUMAB:Acne:1.01), (BICAL UTAMIDE:Acne:0.13), (BISOPROLOL FUMARATE:Acne:0.38), (BRINZOLAMIDE:Acne:0.27), (BUDESONIDE\FORMOTEROL FUMARATE:Acne:0.09), (BUPRENORPHINE HYDROCHLORIDE:Acne:1.42), (CALCITONIN SALMON:Acne:0.83), (CALCIUM ACETATE:Acne:0.78), (CALCIUM\MAGNESIUM:Acne:0.44), (CALCIUM\MAGNESIUM\ZINC:Acne:0.39), (CANAGLIFLOZIN\METFORMIN HYDROCHLORIDE:Acne:0.41), (CANAKINUMAB:Acne:0.46), (CANDESARTAN:Acne:0.83), (CANDESARTAN CILEXETIL:Acne:0.98), (CANDESARTAN CILEXETIL\HYDROCHLOROTHIAZIDE:Acne:0.59), (CARVEDILOL PHOSPHATE:Acne:0.8), (CASANTHRANOL\DOCUSATE SODIUM:Acne:0.11), (CEVIMELINE HYDROCHLORIDE:Acne:0.04), (CHLORPROMAZINE:Acne:0.26), (CHOLESTYRAMINE: Acne:1.86), (CODEINE PHOSPHATE:Acne:0.06), (COLESEVELAM HYDROCHLORIDE:Acne:0.52), (CORTICOTROPIN:Acne:0.34), (DACLIZUMAB:Acne:1.26), (DALTEPARIN:Acne:0.31), (DAPAGLIFLOZIN PROPANEDIOL:Acne:0.19), (DIENOGEST:Acne:0.35), (DIMENHYDRINATE:Acne:1.34), (DI PYRONE:Acne:0), (DOCETAXEL:Acne:0.22), (DOCUSATE CALCIUM:Acne:0.79), (DOFETILIDE:Acne:0.07), (DOXAZOSIN\DOXAZOSIN MESYLATE:Acne:0.18), (DOXEPIN HYDROCHLORIDE:Acne:0.27), (EDOXABAN TOSYLATE:Acne:0.12), (EFINACONAZOLE:Acne:0.4), (EMPAGLIFLOZIN\LINAGLIPTIN: Acne:0.04), (ESTRADIOL HEMIHYDRATE:Acne:0.15), (ESTRADIOL\LEVONORGESTREL:Acne:0.17), (ESTRADIOL\NORETHINDRONE ACETATE:Acne:0.59), (ETRETINATE:Acne:0.12), (EVOLOCUMAB:Acne:0.91), (EZETIMIBE\SIMVASTATIN:Acne:0.76), (FESOTERODINE FUMARATE:Acne:0.64), (FLECAINIDE:Acne:0.52), (FLUOCINOLONE ACETONIDE:Acne:0.35), (FLURAZEPAM\FLURAZEPAM HYDROCHLORIDE:Acne:0.09), (FLUTICASONE:Acne:0.92), (FLUTICASONE FUROATE\VILANTEROL TRIFENATATE:Acne:0.75), (FORMOTEROL FUMARATE\MOMETASONE FUROATE:Acne:0.42), (FULVESTRANT:Acne:0.05), (FUSIDIC ACID:Acne:0.61), (HYDROCHLOROTHIAZIDE\IRBESARTAN:Acne:0.33), (HYDROCODO NE BITARTRATE\IBUPROFEN:Acne:0.47), (IBRUTINIB:Acne:0.74), (IMATINIB MESYLATE:Acne:0.03), (INFLIXIMAB-DYYB:Acne:0.92), (IODINE:Acne:0.2), (IRINOTECAN:Acne:0.38), (ISOSORBIDE:Acne:0.24), (ISOSORBIDE MONONITRATE:Acne:0.62), (LERCANIDIPINE HYDROCHLORIDE:Acne:0.09), (LEVOLEUCOVORIN CALCIUM:Acne:0.58), (LEVOMILNACIPRAN HYDROCHLORIDE:Acne:0.12), (LIOTHYRONINE:Acne:0.4), (LOMITAPIDE MESYLATE:Acne:0.09), (MAGNESIUM GLUCONATE:Acne:0.01), (MEFENAMIC ACID:Acne:0.34), (MEPOLIZUMAB:Acne:0.35), (METHENAMINE HIPPURATE:Acne:0.55), (METHYLPREDNISOLONE ACETATE:Acne:1.58), (MOXIFLOXACIN:Acne:0.22), (NALOXONE HYDROCHLORIDE\OXYCODONE HYDROCHLORIDE:Acne:0.2), (NILOTINIB:Acne:0.89), (NINTEDANIB:Acne:0.03), (OLODA TEROL HYDROCHLORIDE\TIOTROPIUM BROMIDE MONOHYDRATE:Acne:0.38), (OXAZEPAM:Acne:0.19), (PANTOPRAZOLE MAGNESIUM:Acne:1.38), (PEGINTERFERON BETA-1A:Acne:0.28), (PERAMPANEL:Acne:0.1), (PHENYTOIN SODIUM:Acne:0.46), (PILOCARPINE:Acne:0.03), (PIROXICAM:Acne:0.22), (PRAMIPEXOL E:Acne:0.61), (PRAMIPEXOLE DIHYDROCHLORIDE:Acne:0.96), (PRAMOXINE HYDROCHLORIDE:Acne:0.01), (PRAZOSIN HYDROCHLORIDE:Acne:0.2), (PREDNISOLONE SODIUM PHOSPHATE:Acne:0.29), (PROTRIPTYLINE HYDROCHLORIDE:Acne:0.16), (RALOXIFENE HYDROCHLORIDE:Acne:0.09), (RALTEGRAVIR POTASSIUM:Acne:0.77), (RANOLAZINE:Acne:0.41), (RIVASTIGMINE:Acne:0.04), (ROFLU MILAST:Acne:0.84), (SACCHAROMYCES CEREVISIAE:Acne:0.05), (SALMETEROL XINAFOATE:Acne:0.03), (TERIFLUNOMIDE:Acne:0.63), (TRIAMTERENE:Acne:0.56), (UM ECLIDINIUM BROMIDE:Acne:0.09), (UMECLIDINIUM BROMIDEE\VILANTEROL TRIFENATATE:Acne:1.83), (VALGANCICLOVIR HYDROCHLORIDE:Acne:0.27), (OMEPRAZOLE MAGNESIUM:Acne cystic:0.01), (VARENICLINE TARTRATE:Acne cystic:0.24), (COAGULATION FACTOR IX RECOMBINANT HUMAN:Acquired haemophilia:0.05), (CERTOLIZUMAB PEGOL:Acquired immunodeficiency syndrome:0.11), (EMTRICITABINE\RILPIVIRINE HYDROCHLORIDE\TENOFOVIR DISOPROXIL FUMARATE:Acquired immunodeficiency syndrome:0.79), (NAPROXEN:Acquired immunodeficiency syndrome:0.08), (NELFINAVIR:Acquired immunodeficiency syndrome:0.71), (PHENOBARBITAL:Acquired immunodeficiency syndrome:0.03), (ACETAMINOPHEN\OXYCODONE\OXYCODONE HYDROCHLORIDE:Acromegaly:0.09), (ALISKIREN:Acromegaly:0.16), (AMISULPRIDE: Acromegaly:0.08), (AMLODIPINE\VALSARTAN:Acromegaly:0.14), (ATROPINE SULFATE\DIPHENOXYLATE HYDROCHLORIDE:Acromegaly:0.27), (BENAZEPRIL\BENAZEPRIL HYDROCHLORIDE:Acromegaly:0.19), (BRINZOLAMIDE:Acromegaly:0.11), (BUPRENORP HINE HYDROCHLORIDE:Acromegaly:0.08), (BUSPIRONE HYDROCHLORIDE:Acromegaly:0.79), (CALCIUM CARBONATE\VITAMIN D:Acromegaly:0.04), (CANAGLIFLOZIN\METFORMIN HYDROCHLORIDE:Acromegaly:0.39), (CARVEDILOL PHOSPHATE:Acromegaly:0.77), (CERTOLIZUMAB PEGOL:Acromegaly:0.85), (CHLORDIAZEPOXIDE HYDROCHLORIDE\CLIDINIUM BROMIDE:Acromegaly:0.11), (CHOLESTYRAMINE:Acromegaly:1.5), (CLINDAMYCIN\CLI NDAMYCIN PHOSPHATE:Acromegaly:0.63), (CLONIDINE HYDROCHLORIDE:Acromegaly:0.05), (CLOTRIMAZOLE:Acromegaly:0.28), (CLOZAPINE: Acromegaly:0.41), (CORTICOTROPIN:Acromegaly:0.18), (DABIGATRAN:Acromegaly:0.05), (DASATINIB:Acromegaly:0.4), (DEFLAZACORT:Acromegaly:0.23), (DIPHENHYDRAMINE: Acromegaly:0.54), (DOFETILIDE:Acromegaly:0.38), (DOXEPIN HYDROCHLORIDE:Acromegaly:0.19), (EDOXABAN TOSYLATE:Acromegaly:0.32), (ELETRIPTAN HYDROBROMIDE:Acromegaly:0.73), (EMPAGLIFLOZIN\LINAGLIPTIN:Acromegaly:0.95), (EMPAGLIFLOZIN\METFORMIN HYDROCHLORIDE:Acromegaly:0.05), (EPINEPHRINE:Acromegaly:0.17), (EVOLOCUMAB: Acromegaly:1.1), (FESOTERODINE FUMARATE:Acromegaly:0.65), (FUSIDIC ACID:Acromegaly:0.15), (GLYCOPYRRONIUM\INDACATEROL:Acromegaly:0.41), (HERB ALS\TURMERIC:Acromegaly:0.35), (HYDROXYZINE PAMOATE:Acromegaly:0.91), (IBRUTINIB:Acromegaly:0.03), (INSULIN GLULISINE:Acromegaly:0.94), (INSULIN PORK\INSULIN PURIFIED PORK:Acromegaly:0), (IPRATROPIUM BROMIDE:Acromegaly:0.18), (LOMITAPIDE MESYLATE:Acromegaly:0.07), (LUTEIN:Acromegaly:0.03), (METAXALONE:Acromegaly:0.73), (METHENAMINE HIPPURATE:Acromegaly:0.05), (METHYLPREDNISOLONE ACETATE:Acromegaly:1), (METHYLPREDNISOLONE SODIUM SUCCINATE:Acromegaly:0.17), (METRONIDAZOLE\METRONIDAZOLE HYDROCHLORIDE:Acromegaly:0.25), (MOXIFLOXACIN HYDROCHLORIDE:Acromegaly:1.16), (MUPIROCIN\MUPIROCIN CALCIUM:Acromegaly:0), (NALOXONE HYDROCHLORIDE\TILIDINE HYDROCHLORIDE:Acromegaly:0.23), (NATEGLINIDE:Acromegaly:0.38), (NILOTINIB: Acromegaly:0.27), (PANTOPRAZOLE MAGNESIUM:Acromegaly:1.13), (PAZOPANIB HYDROCHLORIDE:Acromegaly:0.03), (PREDNISOLONE\PREDNISOLONE ACETATE\PREDNISOLONE SODIUM SUCCINATE:Acromegaly:0.15), (RALOXIFENE HYDROCHLORIDE:Acromegaly:0.5), (RANOLAZINE:Acromegaly:1.73), (ROPINIROLE HYDROCHLORIDE:Acromegaly:0.59), (SCOPOLAMINE:Acromegaly:0.36), (SUCRALFATE: Acromegaly:0.62), (SUMATRIPTAN:Acromegaly:0.46), (UMECLIDINIUM BROMIDE\VILANTEROL TRIFENATATE:Acromegaly:0.3), (VARENICLINE TARTRATE:Acromegaly:0.86), (VILAZODONE HYDROCHLORIDE:Acromegaly:0.06), (ACETAMINOPHEN\OXYCODONE HYDROCHLORIDE:Actinic keratosis:0.2), (ALBUTEROL SULFATE\IPRATROPIUM BROMIDE:Actinic keratosis:0.12), (AMIODARONE HYDROCHLORIDE:Actinic keratosis:0.39), (AMLODIPINE BESYLATE\HYDROCHLOROTHIAZIDE\OLMESARTAN MEDOXOMIL:Actinic keratosis:0.2), (CALCIUM CITRATE:Actinic keratosis:0.13), (CERTOLIZUMAB PEGOL:Actinic keratosis:0.81), (CLOTRIMAZOLE:Actinic keratosis:0.25), (DASATINIB:Actinic keratosis:0.5), (DICYCLOMINE HYDROCHLORIDE:Actinic keratosis:0.31), (DOFETILIDE:Actinic keratosis:0.07), (ENOXAPARIN:Actinic keratosis:0.45), (ERYTHROPOIETIN:Actinic keratosis:0.03), (ESZOPICLONE:Actinic keratosis:0.09), (EVOLOCUMAB:Actinic keratosis:0.54), (EZETIMIBE\SIMVASTATIN:Actinic keratosis:0.21), (FLECAINIDE:Actinic keratosis:0.02), (FLUCONAZOLE:Actinic keratosis:0.04), (IDELALISIB:Actinic keratosis:0.02), (INSULIN HUMAN:Actinic keratosis:0.12), (IRON:Actinic keratosis:0.81), (LERCANIDIPINE HYDROCHLORIDE:Actinic keratosis:0.1), (METAXALONE:Actinic keratosis:0.57), (METHYLPREDNISOLONE SODIUM SUCCINATE:Actinic keratosis:0.39), (MOXIFLOXACIN HYDROCHLORIDE:Actinic keratosis:0.27), (NEBIVOLOL HYDROCHLORIDE:Actinic keratosis:0.22), (OMEGA-3-ACID ETHYL ESTERS:Actinic keratosis:0.14), (PANTOPRAZOLE MAGNESIUM:Actinic keratosis:0.29), (PONATINIB:Actinic keratosis:0.23), (PROMETHAZINE\PROMETHAZINE HYDROCHLORIDE:Actinic keratosis:0.08), (SACUBITRIL\VALSARTAN:Actinic keratosis:0.69), (SODIUM OXYBATE:Actinic keratosis:0.16), (SOTALOL:Actinic keratosis:0.37), (TERIFLUNOMIDE:Actinic keratosis:0.06), (TIZANIDINE HYDROCHLORIDE:Actinic keratosis:0.12), (UMECLIDINIUM BROMIDE\VILANTEROL TRIFENATATE:Actinic keratosis:0.19), (ACETAMINOPHEN\BUTALBITAL\CAFFEINE:Acute coronary syndrome:0.17), (ACETAMINOPHEN\CAFFEINE\OPIUM:Acute coronary syndrome:0.18), (ACETAMINOPHEN\OXYCODONE\OXYCODONE HYDROCHLORIDE:Acute coronary syndrome:0.19), (AGALSIDASE BETA:Acute coronary syndrome:0.16), (ALBUMIN HUMAN:Acute coronary syndrome:0.13), (ALFUZOSIN HYDROCHLORIDE:Acute coronary syndrome:0.08), (AMLODIPINE BESYLATE\HYDROCHLOROTHIAZIDE\OLMESARTAN MEDOXOMIL:Acute coronary syndrome:0.08), (AMLODIPINE BESYLATE\HYDROCHLOROTHIAZIDE\VALSARTAN:Acute coronary syndrome:0.23), (AMLODIPINE\VALSARTAN:Acute coronary syndrome:0.21), (AMPHETAMINE ASPARTATE\AMPHETAMINE SULFATE\DEXTROAMPHETAMINE SACCHARATE\DEXTROAMPHETAMINE SULFATE:Acute coronary syndrome:0.49),(ARMODAFINIL:Acute coronary syndrome:0.17), (BENAZEPRIL\BENAZEPRIL HYDROCHLORIDE:Acute coronary syndrome:0.37), (BUDESONIDE\FORMOTEROL FUMARATE:Acute coronary syndrome:0.58), (BUPRENORPHINE HYDROCHLORIDE:Acute coronary syndrome:0), (BUSPIRONE HYDROCHLORIDE:Acute coronary syndrome:0.59), (CALCIUM CITRATE:Acute coronary syndrome:0.19), (CANAGLIFLOZIN\METFORMIN HYDROCHLORIDE:Acute coronary syndrome:0.08), (CARFILZOMIB:Acute coronary syndrome:0.54), (CARVEDILOL PHOSPHATE:Acute coronary syndrome:0.97), (CERTOLIZUMAB PEGOL:Acute coronary syndrome:0.49), (CHOLESTYRAMINE:Acute coronary syndrome:0.67), (CLINDAMYCIN:Acute coronary syndrome:0.38), (CLOZAPINE:Acute coronary syndrome:0.14), (DAPAGLIFLOZIN PROPANEDIOL:Acute coronary syndrome:0.3), (DASATINIB:Acute coronary syndrome:1.12), (DIGITOXIN:Acute coronary syndrome:0.6), (DIMENHYDRINATE:Acute coronary syndrome:0.11), (DIVALPROEX SODIUM:Acute coronary syndrome:0.3), (DONEPEZIL:Acute coronary syndrome:0.02), (DRONABINOL:Acute coronary syndrome:0.11), (DUTASTERIDE\TAMSULOSIN HYDROCHLORIDE:Acute coronary syndrome:0.26), (ECONAZOLE:Acute coronary syndrome:0.48), (EDOXABAN TOSYLATE:Acute coronary syndrome:1.34), (ELETRIPTAN HYDROBROMIDE:Acute coronary syndrome:0.17), (ESZOPICLONE:Acute coronary syndrome:0.52), (EVEROLIMUS:Acute coronary syndrome:0.35), (FLECAINIDE:Acute coronary syndrome:1.92), (FLUTICASONE FUROATE:Acute coronary syndrome:0.04), (FORMOTEROL FUMARATE DIHYDRATE\MOMETASONE FUROATE:Acute coronary syndrome:0.17), (GLIMEPIRIDE\ROSIGLITAZONE MALEATE:Acute coronary syndrome:0.44), (HYDROXYZINE PAMOATE:Acute coronary syndrome:0.56), (IDARUCIZUMAB:Acute coronary syndrome:0.84), (IMATINIB MESYLATE:Acute coronary syndrome:0.45), (INSULIN BEEF:Acute coronary syndrome:0.06), (IPRATROPIUM:Acute coronary syndrome:1.13), (LEDIPASVIR\SOFOSBUVIR:Acute coronary syndrome:0.52), (METFORMIN HYDROCHLORIDE\ROSIGLITAZONE MALEATE:Acute coronary syndrome:0.82), (METHYLPREDNISOLONE ACETATE:Acute coronary syndrome:0.13), (NALOXONE HYDROCHLORIDE\OXYCODONE HYDROCHLORIDE:Acute coronary syndrome:1.18), (NATEGLINIDE:Acute coronary syndrome:0.4), (NICARDIPINE:Acute coronary syndrome:0.63), (NILOTINIB:Acute coronary syndrome:1.37), (NORTRIPTYLINE:Acute coronary syndrome:0.31), (OLANZAPINE:Acute coronary syndrome:0.67), (OLODATEROL HYDROCHLORIDE\TIOTROPIUM BROMIDE MONOHYDRATE:Acute coronary syndrome:0.02), (PANTOPRAZOLE MAGNESIUM:Acute coronary syndrome:0.78), (PATIROMER:Acute coronary syndrome:0.09), (PAZOPANIB HYDROCHLORIDE:Acute coronary syndrome:0.08), (PENICILLIN:Acute coronary syndrome:0.43), (PRAMIPEXOLE DIHYDROCHLORIDE:Acute coronary syndrome:0.69), (PREDNISOLONE\PREDNISOLONE ACETATE\PREDNISOLONE SODIUM SUCCINATE:Acute coronary syndrome:0.15), (PROCHLORPERAZINE MALEATE:Acute coronary syndrome:0.07), (PROPAFENONE:Acute coronary syndrome:0.43), (PROPAFENONE HYDROCHLORIDE:Acute coronary syndrome:0.22), (PROPRANOLOL\PROPRANOLOL HYDROCHLORIDE:Acute coronary syndrome:0.11), (RALOXIFENE HYDROCHLORIDE:Acute coronary syndrome:0.11), (ROPINIROLE:Acute coronary syndrome:0.09), (SCOPOLAMINE:Acute coronary syndrome:0.2), (SECUKINUMAB:Acute coronary syndrome:0.07), (SEVELAMER HYDROCHLORIDE:Acute coronary syndrome:0.2), (SODIUM OXYBATE:Acute coronary syndrome:0.23), (SOTALOL HYDROCHLORIDE:Acute coronary syndrome:0.08), (SULFAMETHOXAZOLE:Acute coronary syndrome:0.03), (SUMATRIPTAN\SUMATRIPTAN SUCCINATE:Acute coronary syndrome:0.01), (TAFAMIDIS MEGLUMINE:Acute coronary syndrome:0.27), (TEMAZEPAM:Acute coronary syndrome:1.07), (TESTOSTERONE CYPIONATE:Acute coronary syndrome:0.37), (TETRACYCLINE:Acute coronary syndrome:0.6), (THALIDOMIDE:Acute coronary syndrome:0.07), (TICLOPIDINE:Acute coronary syndrome:0.29), (TIZANIDINE HYDROCHLORIDE:Acute coronary syndrome:0.51), (TOPIRAMATE:Acute coronary syndrome:1), (URSODIOL:Acute coronary syndrome:0.38), (VALGANCICLOVIR HYDROCHLORIDE:Acute coronary syndrome:0.53), (VANCOMYCIN HYDROCHLORIDE:Acute coronary syndrome:0.52), (VASOPRESSIN:Acute coronary syndrome:0.34), (ZONISAMIDE:Acute coronary syndrome:0.17), (ISONIAZID:Acute febrile neutrophilic dermatosis:0.37), (ABACAVIR:Acute HIV infection:0.7), (ACETAMINOPHEN\CODEINE PHOSPHATE:Acute kidney injury:0.61), (ALENDRONIC ACID:Acute kidney injury:0.01), (ANASTROZOLE:Acute kidney injury:0.65), (ATENOLOL:Acute kidney injury:0.68), (BUDESONIDE:Acute kidney injury:1.05), (BUPROPION:Acute kidney injury:0.01), (CANDESARTAN CILEXETIL\HYDROCHLOROTHIAZIDE:Acute kidney injury:0.39), (CEFTRIAXONE SODIUM:Acute kidney injury:0.53), (CHOLESTYRAMINE:Acute kidney injury:0.29), (CLOZAPINE:Acute kidney injury:0.08), (DASATINIB:Acute kidney injury:0.81), (DOXAZOSIN\DOXAZOSIN MESYLATE:Acute kidney injury:0.1), (ENALAPRIL:Acute kidney injury:1.71), (ENALAPRIL MALEATE:Acute kidney injury:0.08), (ESZOPICLONE:Acute kidney injury:0.04), (EVOLOCUMAB:Acute kidney injury:0.33), (EZETIMIBE:Acute kidney injury:0.12), (FLECAINIDE:Acute kidney injury:0.13), (FLUTICASONE PROPIONATE:Acute kidney injury:1.02), (GLIPIZIDE:Acute kidney injury:0.37), (ILOPROST:Acute kidney injury:0.04), (INDAPAMIDE\PERINDOPRIL:Acute kidney injury:0.1), (IPRATROPIUM:Acute kidney injury:0.03), (IRBESARTAN:Acute kidney injury:0.29), (LEDIPASVIR\SOFOSBUVIR:Acute kidney injury:0.22), (LETROZOLE:Acute kidney injury:0.14), (LORATADINE:Acute kidney injury:0.14), (MELOXICAM:Acute kidney injury:0.4), (NILOTINIB:Acute kidney injury:0.67), (NITROFURANTOIN:Acute kidney injury:0.31), (OLANZAPINE:Acute kidney injury:0.04), (PANTOPRAZOLE MAGNESIUM:Acute kidney injury:0.01), (RANOLAZINE:Acute kidney injury:0.33), (SERTRALINE HYDROCHLORIDE:Acute kidney injury:0.88), (TIGECYCLINE:Acute kidney injury:0.04), (TINZAPARIN SODIUM:Acute kidney injury:0.08), (TRAMADOL HYDROCHLORIDE:Acute kidney injury:0.02), (VALGANCICLOVIR HYDROCHLORIDE:Acute kidney injury:0.61), (CASPOFUNGIN:Acute leukaemia:0.03), (CLADRIBINE:Acute leukaemia:0.11), (IBRITUMOMAB TIUXETAN:Acute leukaemia:0.34), (THALIDOMIDE:Acute leukaemia:0.05), (ACARBOSE:Acute lymphocytic leukaemia:0.07), (ACETAMINOPHEN\OXYCODONE\OXYCODONE HYDROCHLORIDE:Acute lymphocytic leukaemia:0.26), (ACETAMINOPHEN\PROPOXYPHENE NAPSYLATE:Acute lymphocytic leukaemia:0.24), (ACLARUBICIN HYDROCHLORIDE:Acute lymphocytic leukaemia:1.45), (ALCOHOL:Acute lymphocytic leukaemia:0.33), (ALENDRONIC ACID:Acute lymphocytic leukaemia:0.12), (ALFACALCIDOL:Acute lymphocytic leukaemia:0.33), (ALUMINUM HYDROXIDE\MAGNESIUM OXIDE\OXETHAZAINE:Acute lymphocytic leukaemia:0.15), (AMISULPRIDE:Acute lymphocytic leukaemia:0.24), (AMMONIUM LACTATE:Acute lymphocytic leukaemia:0.19), (AMPHETAMINE ASPARTATE\AMPHETAMINE SULFATE\DEXTROAMPHETAMINE SACCHARATE\DEXTROAMPHETAMINE SULFATE:Acute lymphocytic leukaemia:0.46), (ATROPINE SULFATE:Acute lymphocytic leukaemia:0.04), (BELATACEPT:Acute lymphocytic leukaemia:0.35), (BENAZEPRIL\BENAZEPRIL HYDROCHLORIDE:Acute lymphocytic leukaemia:0.11), (BEVACIZUMAB:Acute lymphocytic leukaemia:0.06), (BRINCIDOFOVIR:Acute lymphocytic leukaemia:0.88), (BRINZOLAMIDE:Acute lymphocytic leukaemia:0.02), (BUDESONIDE\FORMOTEROL FUMARATE:Acute lymphocytic leukaemia:0.06), (BUPRENORPHINE HYDROCHLORIDE\NALOXONE HYDROCHLORIDE:Acute lymphocytic leukaemia:0.34), (CALCITONIN:Acute lymphocytic leukaemia:0.15), (CALCITONIN SALMON:Acute lymphocytic leukaemia:0.28), (CANAKINUMAB:Acute lymphocytic leukaemia:0.62), (CANDESARTAN CILEXETIL\HYDROCHLOROTHIAZIDE:Acute lymphocytic leukaemia:1.04), (CARISOPRODOL:Acute lymphocytic leukaemia:0.13), (CARVEDILOL PHOSPHATE:Acute lymphocytic leukaemia:0.04), (CEFOTAXIME\CEFOTAXIME SODIUM:Acute lymphocytic leukaemia:0.55), (CERTOLIZUMAB PEGOL:Acute lymphocytic leukaemia:0.98), (CHLORTHALIDONE:Acute lymphocytic leukaemia:0.14), (CLOMIPRAMINE HYDROCHLORIDE:Acute lymphocytic leukaemia:0.04), (CLOZAPINE:Acute lymphocytic leukaemia:0.71), (DABRAFENIB:Acute lymphocytic leukaemia:0.19), (DACLIZUMAB:Acute lymphocytic leukaemia:1.07), (DEXLANSOPRAZOLE:Acute lymphocytic leukaemia:1.03), (DIPYRIDAMOLE:Acute lymphocytic leukaemia:0.16), (DOBUTAMINE HYDROCHLORIDE:Acute lymphocytic leukaemia:0.1), (DOFETILIDE:Acute lymphocytic leukaemia:0), (DOXAZOSIN MESYLATE:Acute lymphocytic leukaemia:0.36), (ELETRIPTAN HYDROBROMIDE:Acute lymphocytic leukaemia:0.61), (ELOTUZUMAB:Acute lymphocytic leukaemia:0.62), (ERYTHROMYCIN:Acute lymphocytic leukaemia:0.86), (ERYTHROPOIETIN:Acute lymphocytic leukaemia:1.06), (ESZOPICLONE:Acute lymphocytic leukaemia:1.03), (EVOLOCUMAB:Acute lymphocytic leukaemia:0.02), (EZETIMIBE\SIMVASTATIN:Acute lymphocytic leukaemia:0.19), (FESOTERODINE FUMARATE:Acute lymphocytic leukaemia:0.38), (FLUINDIONE:Acute lymphocytic leukaemia:0.44), (FLUTICASONE FUROATE:Acute lymphocytic leukaemia:0.08), (FORMOTEROL:Acute lymphocytic leukaemia:0.43), (FORMOTEROL FUMARATE:Acute lymphocytic leukaemia:0.25), (FORMOTEROL FUMARATE DIHYDRATE\MOMETASONE FUROATE:Acute lymphocytic leukaemia:0.14), (FOSFOMYCIN:Acute lymphocytic leukaemia:1.1), (FUSIDIC ACID:Acute lymphocytic leukaemia:0.32), (GLYBURIDE:Acute lymphocytic leukaemia:0.22), (GLYCOPYRRONIUM\INDACATEROL:Acute lymphocytic leukaemia:0.17), (GUSPERIMUS TRIHYDROCHLORIDE:Acute lymphocytic leukaemia:0.49), (HEPARIN CALCIUM:Acute lymphocytic leukaemia:0.26), (HYDROCHLOROTHIAZIDE\TELMISARTAN:Acute lymphocytic leukaemia:0.2), (IBRITUMOMAB TIUXETAN:Acute lymphocytic leukaemia:1.9), (IMMUNE GLOBULIN NOS:Acute lymphocytic leukaemia:0.36), (INDAPAMIDE\PERINDOPRIL:Acute lymphocytic leukaemia:0.52), (INTERFERON GAMMA-1B:Acute lymphocytic leukaemia:0.53), (IRBESARTAN:Acute lymphocytic leukaemia:0.61), (LEDIPASVIR\SOFOSBUVIR:Acute lymphocytic leukaemia:0.59), (LEVALBUTEROL HYDROCHLORIDE:Acute lymphocytic leukaemia:0.63), (LOMUSTINE:Acute lymphocytic leukaemia:0.53), (MAGNESIUM ASPARTATE HYDROCHLORIDE:Acute lymphocytic leukaemia:0), (METAXALONE:Acute lymphocytic leukaemia:0.6), (METHENAMINE HIPPURATE:Acute lymphocytic leukaemia:0.17), (METHENOLONE ENANTHATE:Acute lymphocytic leukaemia:0.66), (METHOTREXATE\METHOTREXATE SODIUM:Acute lymphocytic leukaemia:0.59), (METOLAZONE:Acute lymphocytic leukaemia:0.02), (MIDODRINE:Acute lymphocytic leukaemia:0.19), (MOGAMULIZUMAB:Acute lymphocytic leukaemia:0.87), (MUROMONAB-CD3:Acute lymphocytic leukaemia:0.09), (NADOLOL:Acute lymphocytic leukaemia:0.02), (NIACIN:Acute lymphocytic leukaemia:0.1), (NORTRIPTYLINE:Acute lymphocytic leukaemia:0.34), (PANTOPRAZOLE MAGNESIUM:Acute lymphocytic leukaemia:0.7), (PAROXETINE:Acute lymphocytic leukaemia:0.43), (PAZUFLOXACIN:Acute lymphocytic leukaemia:0.13), (PEMETREXED:Acute lymphocytic leukaemia:0.37), (PEMETREXED DISODIUM:Acute lymphocytic leukaemia:0.15), (PENTOSTATIN:Acute lymphocytic leukaemia:1.31), (PHENYTOIN SODIUM:Acute lymphocytic leukaemia:0.4), (PITAVASTATIN CALCIUM:Acute lymphocytic leukaemia:0.26), (PLERIXAFOR:Acute lymphocytic leukaemia:0.27), (POLYMYXIN B:Acute lymphocytic leukaemia:0.21), (PRAMIPEXOLE:Acute lymphocytic leukaemia:0.07), (PRAMIPEXOLE DIHYDROCHLORIDE:Acute lymphocytic leukaemia:0.28), (PROBIOTICS NOS:Acute lymphocytic leukaemia:0.64), (RABEPRAZOLE SODIUM:Acute lymphocytic leukaemia:0.74), (RANOLAZINE:Acute lymphocytic leukaemia:0.67), (RISEDRONATE SODIUM:Acute lymphocytic leukaemia:0.46), (RIZATRIPTAN BENZOATE:Acute lymphocytic leukaemia:0.34), (ROFLUMILAST:Acute lymphocytic leukaemia:0.37), (SECUKINUMAB:Acute lymphocytic leukaemia:0.32), (SODIUM OXYBATE:Acute lymphocytic leukaemia:0.3), (SUFENTANIL CITRATE:Acute lymphocytic leukaemia:0.08), (SULFAMERAZINE\TRIMETHOPRIM:Acute lymphocytic leukaemia:0.23), (TERBINAFINE HYDROCHLORIDE:Acute lymphocytic leukaemia:0.1), (TERBUTALINE:Acute lymphocytic leukaemia:0.41), (TETRACYCLINE:Acute lymphocytic leukaemia:0.46), (THALIDOMIDE:Acute lymphocytic leukaemia:2.08), (TIMOLOL:Acute lymphocytic leukaemia:0.49), (TIZANIDINE:Acute lymphocytic leukaemia:0.35), (TOLTERODINE TARTRATE:Acute lymphocytic leukaemia:0.11), (TRAMETINIB:Acute lymphocytic leukaemia:0.08), (UMECLIDINIUM BROMIDE\VILANTEROL TRIFENATATE:Acute lymphocytic leukaemia:0.13), (VADASTUXIMAB TALIRINE:Acute lymphocytic leukaemia:0.11), (VARENICLINE TARTRATE:Acute lymphocytic leukaemia:0.28), (VITAMIN A:Acute lymphocytic leukaemia:0.1), (VITAMIN B:Acute lymphocytic leukaemia:0.54), (ZINC:Acute lymphocytic leukaemia:0.33), (FOLIC ACID:Acute lymphocytic leukaemia recurrent:0.14), (IBRITUMOMAB TIUXETAN:Acute lymphocytic leukaemia recurrent:0.36), (THALIDOMIDE:Acute lymphocytic leukaemia recurrent:0.59), (ASPARAGINASE:Acute monocytic leukaemia:0.67), (ACEBUTOLOL HYDROCHLORIDE:Acute myeloid leukaemia:0.02), (ACETAMINOPHEN\CAFFEINE\OPIUM:Acute myeloid leukaemia:0.26), (ACETAMINOPHEN\PROPOXYPHENE HYDROCHLORIDE:Acute myeloid leukaemia:0.5), (ACETAMINOPHEN\PROPOXYPHENE NAPSYLATE:Acute myeloid leukaemia:0.49), (ADO-TRASTUZUMAB EMTANSINE:Acute myeloid leukaemia:0.78), (AGALSIDASE BETA:Acute myeloid leukaemia:0.18), (ALCOHOL:Acute myeloid leukaemia:0.05), (AMANTADINE:Acute myeloid leukaemia:0.74), (AMANTADINE HYDROCHLORIDE:Acute myeloid leukaemia:0.05), (AMILORIDE HYDROCHLORIDE\HYDROCHLOROTHIAZIDE:Acute myeloid leukaemia:0.7), (AMISULPRIDE:Acute myeloid leukaemia:0.34), (AMLODIPINE BESYLATE\HYDROCHLOROTHIAZIDE\OLMESARTAN MEDOXOMIL:Acute myeloid leukaemia:0.28), (AMLODIPINE BESYLATE\VALSARTAN:Acute myeloid leukaemia:0.2), (AMLODIPINE\VALSARTAN:Acute myeloid leukaemia:0.34), (AMMONIUM GLYCYRRHIZATE\CYSTEINE HYDROCHLORIDE\GLYCINE:Acute myeloid leukaemia:0.28), (AMMONIUM LACTATE:Acute myeloid leukaemia:0.5), (ANTIHISTAMINES NOS:Acute myeloid leukaemia:0.01), (ASPIRIN\MAGNESIUM OXIDE:Acute myeloid leukaemia:0.22), (AXITINIB:Acute myeloid leukaemia:0.16), (AZELASTINE:Acute myeloid leukaemia:0.23), (AZITHROMYCIN OR METHOTREXATE SODIUM:Acute myeloid leukaemia:0.09), (BELATACEPT:Acute myeloid leukaemia:0.02), (BENAZEPRIL\BENAZEPRIL HYDROCHLORIDE:Acute myeloid leukaemia:0.67), (BIAPENEM:Acute myeloid leukaemia:0.25), (BRINCIDOFOVIR:Acute myeloid leukaemia:0.04), (BRINZOLAMIDE:Acute myeloid leukaemia:0.89), (BUDESONIDE\FORMOTEROL:Acute myeloid leukaemia:0.16), (BUDESONIDE\FORMOTEROL FUMARATE:Acute myeloid leukaemia:0.42), (BUPRENORPHINE HYDROCHLORIDE:Acute myeloid leukaemia:0.47), (BUPRENORPHINE HYDROCHLORIDE\NALOXONE HYDROCHLORIDE:Acute myeloid leukaemia:0.14), (CABAZITAXEL:Acute myeloid leukaemia:0.08), (CALCITONIN:Acute myeloid leukaemia:0.64), (CALCIUM CITRATE:Acute myeloid leukaemia:0.92), (CALCIUM PANTOTHENATE\CYANOCOBALAMIN\NIACINAMIDE\PYRIDOXINE HYDROCHLORIDE\RIBOFLAVIN\THIAMINE MONONITRATE:Acute myeloid leukaemia:0.14), (CANAKINUMAB:Acute myeloid leukaemia:0.86), (CANDESARTAN CILEXETIL\HYDROCHLOROTHIAZIDE:Acute myeloid leukaemia:1.54), (CARBIMAZOLE:Acute myeloid leukaemia:0.25), (CARVEDILOL PHOSPHATE:Acute myeloid leukaemia:0.72), (CEFOTAXIME\CEFOTAXIME SODIUM:Acute myeloid leukaemia:0.74), (CEFPIROME:Acute myeloid leukaemia:0.11), (CERITINIB:Acute myeloid leukaemia:0.16), (CERTOLIZUMAB PEGOL:Acute myeloid leukaemia:1.06), (CHLORDIAZEPOXIDE HYDROCHLORIDE\CLIDINIUM BROMIDE:Acute myeloid leukaemia:0.03), (CHLORTHALIDONE:Acute myeloid leukaemia:0.86), (CHONDROITIN SULFATE (BOVINE)\GLUCOSAMINE HYDROCHLORIDE:Acute myeloid leukaemia:0.29), (CICLESONIDE:Acute myeloid leukaemia:0.11), (CLONIDINE HYDROCHLORIDE:Acute myeloid leukaemia:0.33), (CLOZAPINE:Acute myeloid leukaemia:0.88), (CORTICOTROPIN:Acute myeloid leukaemia:0.01), (CORTISONE\HYDROCORTISONE:Acute myeloid leukaemia:0.82), (CYCLOPHOSPHAMIDE\CYTARABINE\DEXAMETHASONE\DOXORUB ICIN\METHOTREXATE\VINCRISTINE:Acute myeloid leukaemia:0.91), (CYPROHEPTADINE HYDROCHLORIDE:Acute myeloid leukaemia:0.06), (DABIGATRAN:Acute myeloid leukaemia:0.6), (DABRAFENIB:Acute myeloid leukaemia:0.86), (DABRAFENIB MESYLATE:Acute myeloid leukaemia:0.02), (DACLIZUMAB:Acute myeloid leukaemia:1.19), (DALFAMPRIDINE:Acute myeloid leukaemia:0.03), (DEVICE:Acute myeloid leukaemia:0.04), (DIPYRIDAMOLE:Acute myeloid leukaemia:0.53), (DIPYRONE:Acute myeloid leukaemia:0.55), (DOBUTAMINE HYDROCHLORIDE:Acute myeloid leukaemia:0.53), (DORIPENEM:Acute myeloid leukaemia:0.75), (DOXEPIN:Acute myeloid leukaemia:0.41), (DOXEPIN HYDROCHLORIDE:Acute myeloid leukaemia:0.07), (DRONEDARONE:Acute myeloid leukaemia:0.07), (ECONAZOLE:Acute myeloid leukaemia:1.46), (EDOXABAN TOSYLATE:Acute myeloid leukaemia:0.95), (ELETRIPTAN HYDROBROMIDE:Acute myeloid leukaemia:0.54), (ELOTUZUMAB:Acute myeloid leukaemia:1), (EPIRUBICIN HYDROCHLORIDE:Acute myeloid leukaemia:0.04), (EPLERENONE:Acute myeloid leukaemia:0.69), (ESTROGENS, CONJUGATED\MEDROXYPROGESTERONE ACETATE:Acute myeloid leukaemia:0.22), (EVOLOCUMAB:Acute myeloid leukaemia:0.14), (FERROUS SULFATE\FOLIC ACID:Acute myeloid leukaemia:0.03), (FLUCYTOSINE:Acute myeloid leukaemia:0.23), (FLUDROCORTISONE ACETATE:Acute myeloid leukaemia:0.33), (FLUOCINONIDE:Acute myeloid leukaemia:0.18), (FLUTICASONE FUROATE:Acute myeloid leukaemia:0.55), (FLUVASTATIN SODIUM:Acute myeloid leukaemia:0.35), (FLUVOXAMINE MALEATE:Acute myeloid leukaemia:0.03), (FORMOTEROL FUMARATE DIHYDRATE\MOMETASONE FUROATE:Acute myeloid leukaemia:0.52), (FORMOTEROL FUMARATE\MOMETASONE FUROATE:Acute myeloid leukaemia:0.04), (FOSPHENYTOIN\FOSPHENYTOIN SODIUM:Acute myeloid leukaemia:0.09), (FUSIDIC ACID:Acute myeloid leukaemia:0.89), (GIMERACIL\OTERACIL\TEGAFUR:Acute myeloid leukaemia:0.46), (HEPARIN CALCIUM:Acute myeloid leukaemia:0.68), (HYDROCHLOROTHIAZIDE\TELMISARTAN:Acute myeloid leukaemia:0.51), (IBRITUMOMAB TIUXETAN:Acute myeloid leukaemia:1.78), (IDELALISIB:Acute myeloid leukaemia:1.67), (ILOPROST:Acute myeloid leukaemia:0.08), (INDACATEROL:Acute myeloid leukaemia:0.31), (INOTUZUMAB OZOGAMICIN:Acute myeloid leukaemia:1.65), (INTERFERON ALFA-2A:Acute myeloid leukaemia:0.46), (INTERFERON ALFA-2B:Acute myeloid leukaemia:0), (INTERFERON GAMMA-1B:Acute myeloid leukaemia:0.87), (IRINOTECAN HYDROCHLORIDE:Acute myeloid leukaemia:0.97), (IRON SUCROSE:Acute myeloid leukaemia:0.45), (ISOSORBIDE:Acute myeloid leukaemia:0.89), (KETOROLAC\KETOROLAC TROMETHAMINE:Acute myeloid leukaemia:0.32), (LACTOBACILLUS RHAMNOSUS:Acute myeloid leukaemia:0.42), (LEDIPASVIR\SOFOSBUVIR:Acute myeloid leukaemia:0.62), (LERCANIDIPINE HYDROCHLORIDE:Acute myeloid leukaemia:1.54), (LEVALBUTEROL HYDROCHLORIDE:Acute myeloid leukaemia:1.13), (LEVOLEUCOVORIN CALCIUM:Acute myeloid leukaemia:0.42), (LIOTHYRONINE:Acute myeloid leukaemia:0.05), (LISDEXAMFETAMINE DIMESYLATE:Acute myeloid leukaemia:0.08), (MAGNESIUM ASPARTATE\POTASSIUM ASPARTATE:Acute myeloid leukaemia:0.33), (MELPHALAN HYDROCHLORIDE:Acute myeloid leukaemia:0.02), (METAXALONE:Acute myeloid leukaemia:1.28), (METFORMIN HYDROCHLORIDE\VILDAGLIPTIN:Acute myeloid leukaemia:0.23), (METHENAMINE HIPPURATE:Acute myeloid leukaemia:0.77), (METHOCARBAMOL:Acute myeloid leukaemia:0.94), (METHOTREXATE\METHOTREXATE SODIUM:Acute myeloid leukaemia:0.16), (MIDODRINE HYDROCHLORIDE:Acute myeloid leukaemia:0.09), (MOGAMULIZUMAB:Acute myeloid leukaemia:0.4), (NABUMETONE:Acute myeloid leukaemia:0.31), (NELARABINE:Acute myeloid leukaemia:1.28), (NIRAPARIB:Acute myeloid leukaemia:0.03), (OFATUMUMAB:Acute myeloid leukaemia:0.86), (OLODATEROL HYDROCHLORIDE\TIOTROPIUM BROMIDE MONOHYDRATE:Acute myeloid leukaemia:0.47), (OSPEMIFENE:Acute myeloid leukaemia:0.01), (PALBOCICLIB:Acute myeloid leukaemia:0.07), (PANCRELIPASE:Acute myeloid leukaemia:0.32), (PAZOPANIB HYDROCHLORIDE:Acute myeloid leukaemia:1.32), (PEMETREXED:Acute myeloid leukaemia:0.85), (PEMETREXED DISODIUM:Acute myeloid leukaemia:0.83), (PENTOSTATIN:Acute myeloid leukaemia:1.77), (PENTOXIFYLLINE:Acute myeloid leukaemia:0.13), (PERTUZUMAB:Acute myeloid leukaemia:0.2), (PHENOBARBITAL:Acute myeloid leukaemia:1.43), (PIRARUBICIN:Acute myeloid leukaemia:0.46), (PIROXICAM:Acute myeloid leukaemia:0.11), (PITAVASTATIN CALCIUM:Acute myeloid leukaemia:0.42), (PREDNISOLONE SODIUM PHOSPHATE:Acute myeloid leukaemia:1.01), (PRISTINAMYCIN:Acute myeloid leukaemia:0.22), (PSYLLIUM HUSK:Acute myeloid leukaemia:0.11), (PYRAZINAMIDE:Acute myeloid leukaemia:0.75), (PYRIMETHAMINE:Acute myeloid leukaemia:0.08), (QUINAPRIL:Acute myeloid leukaemia:0.04), (RAMUCIRUMAB:Acute myeloid leukaemia:0.11), (REGORAFENIB:Acute myeloid leukaemia:0.65), (RETINOL:Acute myeloid leukaemia:0.42), (RIVASTIGMINE TARTRATE:Acute myeloid leukaemia:0.28), (RIZATRIPTAN BENZOATE:Acute myeloid leukaemia:0.62), (ROFLUMILAST:Acute myeloid leukaemia:0.79), (ROPINIROLE HYDROCHLORIDE:Acute myeloid leukaemia:0.72), (SECUKINUMAB:Acute myeloid leukaemia:0.2), (SODIUM OXYBATE:Acute myeloid leukaemia:0.41), (SUFENTANIL CITRATE:Acute myeloid leukaemia:0.28), (SULFAMERAZINE\TRIMETHOPRIM:Acute myeloid leukaemia:0.03), (TAMOXIFEN CITRATE:Acute myeloid leukaemia:0.03), (TEDUGLUTIDE\WATER:Acute myeloid leukaemia:0.22), (TERBUTALINE:Acute myeloid leukaemia:0.84), (TISAGENLECLEUCEL:Acute myeloid leukaemia:0.64), (TRABECTEDIN:Acute myeloid leukaemia:0.31), (UMECLIDINIUM BROMIDE\VILANTEROL TRIFENATATE:Acute myeloid leukaemia:0.56), (VARENICLINE TARTRATE:Acute myeloid leukaemia:0.63), (VEMURAFENIB:Acute myeloid leukaemia:0.68), (ZONISAMIDE:Acute myeloid leukaemia:0.54), (BLINATUMOMAB:Acute myeloid leukaemia recurrent:0.15), (AGALSIDASE BETA:Acute myocardial infarction:0.12), (ALBUMIN HUMAN:Acute myocardial infarction:0.07), (AMLODIPINE\VALSARTAN:Acute myocardial infarction:0.14), (ANASTROZOLE:Acute myocardial infarction:1.13), (BENAZEPRIL HYDROCHLORIDE:Acute myocardial infarction:0.44), (BISACODYL OR DOCUSATE SODIUM:Acute myocardial infarction:0.34), (BUDESONIDE\FORMOTEROL FUMARATE:Acute myocardial infarction:0.09), (BUPROPION:Acute myocardial infarction:0.18), (CALCIUM\VITAMIN D:Acute myocardial infarction:0.01), (CHOLESTYRAMINE:Acute myocardial infarction:0.04), (CLINDAMYCIN:Acute myocardial infarction:0.02), (CLINDAMYCIN\CLINDAMYCIN PHOSPHATE:Acute myocardial infarction:1.11), (CLOTRIMAZOLE:Acute myocardial infarction:0.35), (COAGULATION FACTOR IX HUMAN\COAGULATION FACTOR VII HUMAN\COAGULATION FACTOR X HUMAN\PROTEIN C\PROTEIN S HUMANPROTHROMBIN:Acute myocardial infarction:0.05), (CORTISONEHYDROCORTISONE:Acute myocardial infarction:0.37), (CYCLOBENZAPRINE:Acute myocardial infarction:0.05), (DASATINIB:Acute myocardial infarction:0.84), (DEVICE:Acute myocardial infarction:0.2), (DEXLANSOPRAZOLE:Acute myocardial infarction:0.49), (DEXTROSE\HEPARIN:Acute myocardial infarction:0.27), (DOFETILIDE:Acute myocardial infarction:0.18), (DROTRECOGIN ALFA (ACTIVATED):Acute myocardial infarction:0.06), (ESZOPICLONE:Acute myocardial infarction:0.28), (ETORICOXIB:Acute myocardial infarction:0.32), (FESOTERODINE FUMARATE:Acute myocardial infarction:0.16), (FLECAINIDE:Acute myocardial infarction:1.33), (FLUINDIONE:Acute myocardial infarction:0.12), (FLUTICASONE:Acute myocardial infarction:0.17), (GLIMEPIRIDE\ROSIGLITAZONE MALEATE:Acute myocardial infarction:0.39), (HYDROCHLOROTHIAZIDE\TELMISARTAN:Acute myocardial infarction:0.19), (HYDROXYCHLOROQUINE SULFATE:Acute myocardial infarction:0.3), (HYDROXYUREA:Acute myocardial infarction:0.61), (IDARUCIZUMAB:Acute myocardial infarction:1.56), (ILOPROST:Acute myocardial infarction:0.87), (INDAPAMIDE\PERINDOPRIL:Acute myocardial infarction:1.15), (LEDIPASVIR\SOFOSBUVIR:Acute myocardial infarction:0.65), (LEVALBUTEROL HYDROCHLORIDE:Acute myocardial infarction:0.21), (LITHIUM CARBONATE:Acute myocardial infarction:0.15), (METFORMIN HYDROCHLORIDE\ROSIGLITAZONE MALEATE:Acute myocardial infarction:0.69), (MIDODRINE:Acute myocardial infarction:0.23), (MOXIFLOXACIN HYDROCHLORIDE:Acute myocardial infarction:0.89), (NALOXONE HYDROCHLORIDE\OXYCODONE HYDROCHLORIDE:Acute myocardial infarction:0.47), (NICARDIPINE:Acute myocardial infarction:0.55), (NOREPINEPHRINE BITARTRATE:Acute myocardial infarction:0.51), (OLANZAPINE:Acute myocardial infarction:0.09), (OLODATEROL HYDROCHLORIDE\TIOTROPIUM BROMIDE MONOHYDRATE:Acute myocardial infarction:0.22), (PARICALCITOL:Acute myocardial infarction:0.61), (PHENOBARBITAL:Acute myocardial infarction:0.17), (PILSICAINIDE HYDROCHLORIDE:Acute myocardial infarction:0.09), (POLYETHYLENE GLYCOLS:Acute myocardial infarction:1.02), (PRAMIPEXOLE DIHYDROCHLORIDE:Acute myocardial infarction:0.07), (PROPAFENONE:Acute myocardial infarction:0.37), (PROPAFENONE HYDROCHLORIDE:Acute myocardial infarction:0.25), (RABEPRAZOLE:Acute myocardial infarction:0.1), (ROSIGLITAZONE MALEATE:Acute myocardial infarction:0.82), (SCOPOLAMINE:Acute myocardial infarction:0.04), (SELEXIPAG:Acute myocardial infarction:0.11), (SEVELAMER HYDROCHLORIDE:Acute myocardial infarction:0.22), (TAFAMIDIS MEGLUMINE:Acute myocardial infarction:0.19), (THALIDOMIDE:Acute myocardial infarction:0.49), (TOPIRAMATE:Acute myocardial infarction:0.71), (TRANEXAMIC ACID:Acute myocardial infarction:0.14), (VALGANCICLOVIR HYDROCHLORIDE:Acute myocardial infarction:0.18), (VANCOMYCIN HYDROCHLORIDE:Acute myocardial infarction:0.18), (ASPARAGINASE:Acute promyelocytic leukaemia:0.04), (BUMETANIDE:Acute respiratory failure:0.07), (ATORVASTATIN CALCIUM:Acute sinusitis:0.4), (CERTOLIZUMAB PEGOL:Acute sinusitis:0.8), (ENALAPRIL:Acute sinusitis:0.37), (ESZOPICLONE:Acute sinusitis:0.04), (EVOLOCUMAB:Acute sinusitis:0.03), (IBRUTINIB:Acute sinusitis:0.78), (PRAVASTATIN SODIUM:Acute sinusitis:0.11), (TERIFLUNOMIDE:Acute sinusitis:0.18), (TORSEMIDE:Acute sinusitis:0.04), (ACETAMINOPHEN\CODEINE PHOSPHATE:Addison's disease:0.17), (AMITRIPTYLINE:Addison's disease:0.13), (ATENOLOL:Addison's disease:0.42), (BUMETANIDE:Addison's disease:0.16), (CALCIUM CARBONATE\CHOLECALCIFEROL:Addison's disease:0.25), (CERTOLIZUMAB PEGOL:Addison's disease:0.34), (CLINDAMYCIN\CLINDAMYCIN PHOSPHATE:Addison's disease:0.12), (CYCLOBENZAPRINE:Addison's disease:0.08), (DALFAMPRIDINE:Addison's disease:0.25), (DICLOFENAC SODIUM:Addison's disease:0.06), (ELETRIPTAN HYDROBROMIDE:Addison's disease:0.12), (EPINEPHRINE:Addison's disease:0.26), (ESZOPICLONE:Addison's disease:0.09), (EVEROLIMUS:Addison's disease:0.14), (EVOLOCUMAB:Addison's disease:0.26), (IBRUTINIB:Addison's disease:0.41), (ILOPROST:Addison's disease:0.06), (LISDEXAMFETAMINE DIMESYLATE:Addison's disease:0.03), (LOVASTATIN:Addison's disease:0.37), (MELATONIN:Addison's disease:0.29), (POLYETHYLENE GLYCOLS:Addison's disease:0.02), (PRAVASTATIN SODIUM:Addison's disease:0.21), (RAMIPRIL:Addison's disease:0.08), (SACUBITRIL\VALSARTAN:Addison's disease:0.03), (TAMSULOSIN:Addison's disease:0.23), (TERIFLUNOMIDE:Addison's disease:0.57), (TOPIRAMATE:Addison's disease:0.87), (VARENICLINE TARTRATE:Addison's disease:0.29), (AMIODARONE:Adenocarcinoma:0.56), (ANASTROZOLE:Adenocarcinoma:1.38), (BUMETANIDE:Adenocarcinoma:0.37), (CALCITRIOL:Adenocarcinoma:0.1), (CALCIUM\VITAMIN D:Adenocarcinoma:0.02), (DASATINIB:Adenocarcinoma:0.59), (DICLOFENAC:Adenocarcinoma: 0.3), (DIGOXIN:Adenocarcinoma:0.74), (EVOLOCUMAB:Adenocarcinoma:0.07), (FLUTIC ASONE PROPIONATE:Adenocarcinoma:0.49), (IBRUTINIB:Adenocarcinoma:0.3), (INDAPAMIDE\PE RINDOPRIL:Adenocarcinoma:0.1), (LEDIPASVIR\SOFOSBUVIR:Adenocarcinoma:0), (LERC ANIDIPINE HYDROCHLORIDE:Adenocarcinoma:0.05), (LOVASTATIN:Adenocarcinoma:0.31), (MAGNE SIUM:Adenocarcinoma:0.06), (MELOXICAM:Adenocarcinoma:0), (NIFEDIPINE:Adenocarcinoma: 0.14), (NILOTINIB:Adenocarcinoma:0.65), (NITROFURANTOIN:Adenocarcinoma:0.04), (O LAPARIB:Adenocarcinoma:0.28), (PERTUZUMAB:Adenocarcinoma:0.47), (PRAVASTATIN: Adenocarcinoma:0.21), (SERTRALINE HYDROCHLORIDE:Adenocarcinoma:0), (SOTALOL:Adenocarcinoma:0.13), (CETUXIMAB:A denocarcinoma gastric:0.56), (ETHAMBUTOL HYDROCHLORIDE:Adenocarcinoma gastric:0.82), (NILOTINIB:Adenocarcinoma gastric:0.13), (PANITUMUMAB:Adenocarcinoma gastric:0.17), (AMIODARONE:Adenocarcinoma of colon:0.17), (BUMETANIDE:Adenocarcinoma of colon:0.03), (CALCIUM CARBONATE\CHOLECALCIFEROL:Adenocarcinoma of colon:0.17), (DILTIAZEM HYDROCHLORIDE:Adenocarcinoma of colon:0.12), (FERROUS SULFATE\FERROUS SULFATE, DRIED:Adenocarcinoma of colon:0.06), (FLUTICASONE PROPIONATE:Adenocarcinoma of colon:0.1), (ANASTROZOLE:Adenocarcinoma pancreas:0.4), (BUDESONIDE:Adenocarcinoma pancreas:0.47), (CERTOLIZUMAB PEGOL:Adenocarcinoma pancreas:0.08), (CLONIDINE:Adenocarcinoma pancreas:0.24), (CLOTRIMAZOLE:Adenocarcinoma pancreas:0.01), (DEXLANSOPRAZOLE:Adenocarcinoma pancreas:0.58), (DICLOFENAC:Adenocarcinoma pancreas:0.3), (DIPHENHYDRAMINE:Adenocarcinoma pancreas:0.41), (EPINEPHRINE:Adenocarcinoma pancreas:0.1), (EPIRUBICIN:Adenocarcinoma pancreas:0.05), (EVEROLIMUS:Adenocarcinoma pancreas:0.24), (EVOLOCUMAB:Adenocarcinoma pancreas:0.13), (FERROUS SULFATE\FERROUS SULFATE, DRIED:Adenocarcinoma pancreas:0.76), (INDAPAMIDE\PERINDOPRIL:Adenocarcinoma pancreas:0.12), (ISOSORBIDE MONONITRATE:Adenocarcinoma pancreas:0.07), (NIFEDIPINE:Adenocarcinoma pancreas:0.46), (NILOTINIB:Adenocarcinoma pancreas:0.56), (OMEPRAZOLE MAGNESIUM:Adenocarcinoma pancreas:0.41), (PANITUMUMAB:Adenocarcinoma pancreas:0.24), (PAZOPANIB HYDROCHLORIDE:Adenocarcinoma pancreas:0.2), (PRAVASTATIN SODIUM:Adenocarcinoma pancreas:0.13), (TIMOLOL:Adenocarcinoma pancreas:0.08), (TIOTROPIUM BROMIDE MONOHYDRATE:Adenocarcinoma pancreas:0.06), (VERAPAMIL HYDROCHLORIDE:Adenocarcinoma pancreas:0.49), (ZOLPIDEM TARTRATE:Adenocarcinoma pancreas:0.07), (ACETAMINOPHEN\OXYCODONE\OXYCODONE HYDROCHLORIDE:Adjuvant therapy:0.1), (ALCOHOL:Adjuvant therapy:0.45), (ALENDRONIC ACID:Adjuvant therapy:0.1), (AMISULPRIDE:Adjuvant therapy:0.41), (AZITHROMYCIN DIHYDRATE:Adjuvant therapy:0.03), (BENDAMUSTINE HYDROCHLORIDE:Adjuvant therapy:1.05), (BLINATUMOMAB:Adjuvant therapy:0.41), (BUPRENORPHINE HYDROCHLORIDE:Adjuvant therapy:0.55), (CALCIUM ACETATE:Adjuvant therapy:0.3), (CALCIUM CITRATE:Adjuvant therapy:0.42), (CANAKINUMAB:Adjuvant therapy:0.04), (CANDESARTAN:Adjuvant therapy:0.54), (CANDESARTAN CILEXETIL:Adjuvant therapy:0.74), (CARISOPRODOL:Adjuvant therapy:0.12), (CARVEDILOL PHOSPHATE:Adjuvant therapy:0.18), (CHLORAMBUCIL:Adjuvant therapy:0.25), (CIPROFLOXACIN HYDROCHLORIDE:Adjuvant therapy:0.48), (CLINDAMYCIN:Adjuvant therapy:0.22), (CLOTRIMAZOLE:Adjuvant therapy:0.41), (CODEINE:Adjuvant therapy:0.6), (DACLIZUMAB:Adjuvant therapy:0.23), (DALFAMPRIDINE:Adjuvant therapy:0.76), (DASATINIB:Adjuvant therapy:1.34), (DEVICE:Adjuvant therapy:0.12), (DIMENHYDRINATE:Adjuvant therapy:0.22), (DOXAZOSIN MESYLATE:Adjuvant therapy:0.18), (DUTASTERIDE:Adjuvant therapy:0.6), (ENALAPRIL MALEATE:Adjuvant therapy:0.55), (EVOLOCUMAB:Adjuvant therapy:0.82), (EZETIMIBE\SIMVASTATIN:Adjuvant therapy:0.09), (FLECAINIDE:Adjuvant therapy:0.32), (FLUTICASONE:Adjuvant therapy:0.2), (HYDROCHLOROTHIAZIDE\IRBESARTAN:Adjuvant therapy:0.17), (HYDROXYCHLOROQUINE DIPHOSPHATE:Adjuvant therapy:0), (IMIPRAMINE HYDROCHLORIDE:Adjuvant therapy:0.15), (INDAPAMIDE:Adjuvant therapy:0.03), (INDAPAMIDE\PERINDOPRIL:Adjuvant therapy:0.09), (INFLUENZA VIRUS VACCINE:Adjuvant therapy:0.06), (IPRATROPIUM:Adjuvant therapy:0.44), (IRINOTECAN:Adjuvant therapy:0.47), (IRINOTECAN HYDROCHLORIDE:Adjuvant therapy:0.05), (LEDIPASVIR\SOFOSBUVIR:Adjuvant therapy:0.69), (LEVALBUTEROL HYDROCHLORIDE:Adjuvant therapy:0.31), (LIDOCAINE\PRILOCAINE:Adjuvant therapy:0.3), (LITHIUM CARBONATE:Adjuvant therapy:0.68), (METAXALONE:Adjuvant therapy:0.64), (METHENAMINE HIPPURATE:Adjuvant therapy:0.24), (METHYLPREDNISOLONE ACETATE:Adjuvant therapy:0.27), (MICAFUNGIN:Adjuvant therapy:0.2), (MYCOPHENOLATE MOFETIL\MYCOPHENOLATE MOFETIL HYDROCHLORIDE:Adjuvant therapy:0.02), (NALOXONE HYDROCHLORIDE\OXYCODONE HYDROCHLORIDE:Adjuvant therapy:0.21), (OLAPARIB:Adjuvant therapy:0.13), (PANTOPRAZOLE MAGNESIUM:Adjuvant therapy:0.11), (PAZOPANIB HYDROCHLORIDE:Adjuvant therapy:0.75), (PEMETREXED:Adjuvant therapy:0.27), (PENICILLIN:Adjuvant therapy:0.53), (PHENOBARBITAL:Adjuvant therapy:0.34), (PITAVASTATIN CALCIUM:Adjuvant therapy:0.07), (PONATINIB:Adjuvant therapy:0.63), (RABEPRAZOLE:Adjuvant therapy:0.07), (RIZATRIPTAN BENZOATE:Adjuvant therapy:0.26), (SACUBITRIL\VALSARTAN:Adjuvant therapy:1.07), (SCOPOLAMINE:Adjuvant therapy:0.55), (SULFAMETHOXAZOLE:Adjuvant therapy:0.33), (SUMATRIPTAN\SUMATRIPTAN SUCCINATE:Adjuvant therapy:0.57), (TERIFLUNOMIDE:Adjuvant therapy:0.33), (TIOTROPIUM:Adjuvant therapy:0.02), (TOCILIZUMAB:Adjuvant therapy:0.36), (TOLTERODINE TARTRATE:Adjuvant therapy:0.07), (UMECLIDINIUM BROMIDE\VILANTEROL TRIFENATATE:Adjuvant therapy:0.08), (VALGANCICLOVIR HYDROCHLORIDE:Adjuvant therapy:0.13), (VANCOMYCIN HYDROCHLORIDE:Adjuvant therapy:0.34), (VINDESINE SULFATE:Adjuvant therapy:0.13), (ZONISAMIDE:Adjuvant therapy:0.26), (NAPROXEN:Adrenal disorder:0), (ADO-TRASTUZUMAB EMTANSINE:Adrenal insufficiency:0.14), (ALENDRONIC ACID:Adrenal insufficiency:0.23), (ANASTROZOLE:Adrenal insufficiency:1.27), (APREPITANT:Adrenal insufficiency:0.08), (BICALUTAMIDE:Adrenal insufficiency:0.52), (BUPROPION:Adrenal insufficiency:0.6), (CALCIUM CARBONATE\VITAMIN D:Adrenal insufficiency:0.07), (CANDESARTAN CILEXETIL\HYDROCHLOROTHIAZIDE:Adrenal insufficiency:0.58), (CARVEDILOL PHOSPHATE:Adrenal insufficiency:0.17), (CEFOTAXIME SODIUM:Adrenal insufficiency:0.14), (CETUXIMAB:Adrenal insufficiency:0.05), (CILASTATIN SODIUM\IMIPENEM:Adrenal insufficiency:0.31), (CLOTRIMAZOLE:Adrenal insufficiency:0.51), (CLOZAPINE:Adrenal insufficiency:0.19), (DACLIZUMAB:Adrenal insufficiency:0.51), (DASATINIB:Adrenal insufficiency:0.55), (DESLORATADINE:Adrenal insufficiency:0.25), (DEVICE:Adrenal insufficiency:0.04), (DIMENHYDRINATE:Adrenal insufficiency:0.51), (DIVALPROEX SODIUM:Adrenal insufficiency:1.04), (DOCUSATE:Adrenal insufficiency:0.26), (DOCUSATE CALCIUM:Adrenal insufficiency:0.07), (DOXAZOSIN\DOXAZOSIN MESYLATE:Adrenal insufficiency:0.21), (ELETRIPTAN HYDROBROMIDE:Adrenal insufficiency:0.61), (ENALAPRIL MALEATE:Adrenal insufficiency:0.81), (ETORICOXIB:Adrenal insufficiency:0.01), (EVOLOCUMAB:Adrenal insufficiency:0.59), (EZETIMIBE\SIMVASTATIN:Adrenal insufficiency:0.21), (FLECAINIDE:Adrenal insufficiency:0.45), (FLUTICASONE FUROATE\VILANTEROL TRIFENATATE:Adrenal insufficiency:0.33), (GLICLAZIDE:Adrenal insufficiency:0.23), (GLYBURIDE:Adrenal insufficiency:0.3), (HYDROXYCHLOROQUINE DIPHOSPHATE:Adrenal insufficiency:0.08), (IDELALISIB:Adrenal insufficiency:0.04), (ILOPROST:Adrenal insufficiency:0.38), (IMIPRAMINE HYDROCHLORIDE:Adrenal insufficiency:0.22), (INDAPAMIDE\PERINDOPRIL:Adrenal insufficiency:0.41), (IRINOTECAN HYDROCHLORIDE:Adrenal insufficiency:0.19), (ISONIAZID:Adrenal insufficiency:0.49), (ISOSORBIDE:Adrenal insufficiency:0.3), (LABETALOL\LABETALOL HYDROCHLORIDE:Adrenal insufficiency:0.82), (LACOSAMIDE:Adrenal insufficiency:0.35), (LEDIPASVIR\SOFOSBUVIR:Adrenal insufficiency:0.36), (LERCANIDIPINE HYDROCHLORIDE:Adrenal insufficiency:0.45), (LIDOCAINE\PRILOCAINE:Adrenal insufficiency:0.31), (LOVASTATIN:Adrenal insufficiency:0.64), (MESNA:Adrenal insufficiency:0.03), (METHENAMINE HIPPURATE:Adrenal insufficiency:0.11), (METHYLPREDNISOLONE ACETATE:Adrenal insufficiency:0.13), (METRONIDAZOLE\METRONIDAZOLE HYDROCHLORIDE:Adrenal insufficiency:0.73), (MOXIFLOXACIN HYDROCHLORIDE:Adrenal insufficiency:0.25), (NADOLOL:Adrenal insufficiency:0.11), (NIACIN:Adrenal insufficiency:0.33), (PANTOPRAZOLE:Adrenal insufficiency:1.75), (PAROXETINE\PAROXETINE HYDROCHLORIDE:Adrenal insufficiency:0.49), (PRAMIPEXOLE:Adrenal insufficiency:0.34), (PRIMIDONE:Adrenal insufficiency:0.59), (PYRIDOXINE:Adrenal insufficiency:0.23), (QUETIAPINE:Adrenal insufficiency:0.35), (ROFLUMILAST:Adrenal insufficiency:0.28), (SACUBITRIL\VALSARTAN:Adrenal insufficiency:0.66), (SULFAMETHOXAZOLE:Adrenal insufficiency:0.12), (SUMATRIPTAN:Adrenal insufficiency:0.29), (THALIDOMIDE:Adrenal insufficiency:0.73), (TIOTROPIUM:Adrenal insufficiency:0.04), (TIZANIDINE:Adrenal insufficiency:0.26), (TOCILIZUMAB:Adrenal insufficiency:0.45), (TRIAMTERENE:Adrenal insufficiency:0.18), (UMECLIDINIUM BROMIDE\VILANTEROL TRIFENATATE:Adrenal insufficiency:0.04), (VALGANCICLOVIR HYDROCHLORIDE:Adrenal insufficiency:0.68), (VALSARTAN:Adrenal insufficiency:1.27), (PYRAZINAMIDE:Adrenocortical insufficiency acute:0.56), (PYRAZINAMIDE:Adrenocorticotropic hormone deficiency:0.56), (PYRAZINAMIDE:Adult T-cell lymphoma/leukaemia:0.59), (ADO-TRASTUZUMAB EMTANSINE:Adverse drug reaction:0.01), (ALCOHOL:Adverse drug reaction:0.45), (AMITRIPTYLINE:Adverse drug reaction:0.42), (BUMETANIDE:Adverse drug reaction:0.2), (BUPRENORPHINE HYDROCHLORIDE\NALOXONE HYDROCHLORIDE:Adverse drug reaction:0.22), (CEPHALEXIN:Adverse drug reaction:0.21), (CHOLESTYRAMINE:Adverse drug reaction:0.07), (CYCLOBENZAPRINE HYDROCHLORIDE:Adverse drug reaction:0.33), (DALFAMPRIDINE:Adverse drug reaction:0.1), (DEXLANSOPRAZOLE:Adverse drug reaction:0.72), (DICLOFENAC:Adverse drug reaction:0.83), (DILTIAZEM HYDROCHLORIDE:Adverse drug reaction:0.48), (ELETRIPTAN HYDROBROMIDE:Adverse drug reaction:0.18), (EPIRUBICIN:Adverse drug reaction:0.47), (ESZOPICLONE:Adverse drug reaction:0.44), (EVEROLIMUS:Adverse drug reaction:0.41), (FENOFIBRATE:Adverse drug reaction:0.19), (FERROUS SULFATE\FERROUS SULFATE, DRIED:Adverse drug reaction:0.37), (FULVESTRANT:Adverse drug reaction:0.81), (IBRUTINIB:Adverse drug reaction:0.59), (IRINOTECAN:Adverse drug reaction:0.29), (LEDIPASVIR\SOFOSBUVIR:Adverse drug reaction:0.27), (METAXALONE:Adverse drug reaction:0.01), (METRONIDAZOLE:Adverse drug reaction:0.81), (PAROXETINE:Adverse drug reaction:0.66), (PHENOBARBITAL:Adverse drug reaction:0.31), (PRAMIPEXOLE:Adverse drug reaction:0.21), (PRAVASTATIN:Adverse drug reaction:0.35), (PRAVASTATIN SODIUM:Adverse drug reaction:0.47), (PRIMIDONE:Adverse drug reaction:0.04), (PROGESTERONE:Adverse drug reaction:0.49), (SACUBITRIL\VALSARTAN:Adverse drug reaction:0.33), (SECUKINUMAB:Adverse drug reaction:0.9), (TERIFLUNOMIDE:Adverse drug reaction:0.24), (VERAPAMIL HYDROCHLORIDE:Adverse drug reaction:0.4), (VITAMIN B:Adverse drug reaction:0.04), (ZONISAMIDE:Adverse drug reaction:0.23), (ACENOCOUMAROL:Adverse event:0.14), (ACETAMINOPHEN\BUTALBITAL\CAFFEINE:Adverse event:0.55), (ACETAMINOPHEN\OXYCODONE\OXYCODONE HYDROCHLORIDE:Adverse event:0.5), (AGALSIDASE BETA:Adverse event:0.03), (AMANTADINE:Adverse event:0.28), (AMLODIPINE\PERINDOPRIL:Adverse event:0.03), (AMLODIPINE\VALSARTAN:Adverse event:0.09),(ARMODAFINIL:Adverse event:0.65), (AZELASTINE:Adverse event:0.04), (BECLOMETHASONE DIPROPIONATE:Adverse event:0.26), (BENDAMUSTINE HYDROCHLORIDE:Adverse event:0.15), (BRINZOLAMIDE:Adverse event:0.75), (BUDESONIDE\FORMOTEROL FUMARATE:Adverse event:0.19), (BUPRENORPHINE HYDROCHLORIDE\NALOXONE HYDROCHLORIDE:Adverse event:0.35), (CABAZITAXEL:Adverse event:0.22), (CALCITONIN:Adverse event:0.06), (CALCIUM CARBONATE\CHOLECALCIFEROL\MAGNESIUM CARBONATE:Adverse event:0.31), (CALCIUM CARBONATE\VITAMIN D:Adverse event:0.38), (CALCIUM CITRATE:Adverse event:0.63), (CANAKINUMAB:Adverse event:0.58), (CAPTOPRIL:Adverse event:0.16), (CARVEDILOL PHOSPHATE:Adverse event:0.86), (CASPOFUNGIN:Adverse event:0.28), (CEFOTAXIME SODIUM:Adverse event:0.72), (CERTOLIZUMAB PEGOL:Adverse event:1.69), (CHLORAMBUCIL:Adverse event:0.34), (CHLORTHALIDONE:Adverse event:0.41), (CHONDROITIN SULFATE (BOVINE)\GLUCOSAMINE HYDROCHLORIDE:Adverse event:0.01), (CIMETIDINE:Adverse event:0.64), (CLOMIPRAMINE HYDROCHLORIDE:Adverse event:0.09), (DABIGATRAN:Adverse event:0.28), (DABRAFENIB:Adverse event:0.07), (DACLIZUMAB:Adverse event:0.3), (DALFAMPRIDINE:Adverse event:0.25), (DALTEPARIN:Adverse event:0.66), (DANAZOL:Adverse event:0.3), (DEFLAZACORT:Adverse event:0.33), (DEVICE:Adverse event:0.24), (DIPYRONE:Adverse event:0.32), (DOLASETRON MESYLATE:Adverse event:0.03), (DONEPEZIL:Adverse event:0), (DOXEPIN HYDROCHLORIDE:Adverse event:0.09), (ELETRIPTAN HYDROBROMIDE:Adverse event:0.8), (ESTROGENS, CONJUGATED\MEDROXYPROGESTERONE ACETATE:Adverse event:0.21), (ETORICOXIB:Adverse event:0.5), (EVEROLIMUS:Adverse event:1.46), (EVOLOCUMAB:Adverse event:0.45), (FINGOLIMOD HYDROCHLORIDE:Adverse event:0.08), (FLECAINIDE:Adverse event:0.79), (FLUINDIONE:Adverse event:0.34), (FLUOXETINE\FLUOXETINE HYDROCHLORIDE:Adverse event:0.79), (FLUTICASONE FUROATE\VILANTEROL TRIFENATATE:Adverse event:0.07), (FORMOTEROL FUMARATE:Adverse event:0.13), (FORMOTEROL FUMARATE DIHYDRATE\MOMETASONE FUROATE:Adverse event:0.18), (FOSPHENYTOIN\FOSPHENYTOIN SODIUM:Adverse event:0.04), (GEFITINIB:Adverse event:0.01), (GEMFIBROZIL:Adverse event:0.01), (GLYCOPYRRONIUM\ANDACATEROL:Adverse event:0.45), (HEPARIN CALCIUM:Adverse event:0.21), (HYDROCHLOROTHIAZIDE\TELMISARTAN:Adverse event:0.15), (HYDROXYCHLOROQUINE DIPHOSPHATE:Adverse event:0.26), (IBRITUMOMAB TIUXETAN:Adverse event:0.39), (IDELALISIB:Adverse event:0.4), (ILOPROST:Adverse event:0.3), (IMATINIB MESYLATE:Adverse event:0.99), (IMIPENEM:Adverse event:0.39), (INOTUZUMAB OZOGAMICIN:Adverse event:0.14), (IRINOTECAN:Adverse event:0.28), (IRINOTECAN HYDROCHLORIDE:Adverse event:0.58), (ISOSORBIDE:Adverse event:0.69), (ITRACONAZOLE:Adverse event:0.7), (LABETALOL HYDROCHLORIDE:Adverse event:0.4), (LACTOBACILLUS RHAMNOSUS:Adverse event:0.09), (LAFUTIDINE:Adverse event:0.11), (LAPATINIB DITOSYLATE:Adverse event:0.22), (LEDIPASVIR\SOFOSBUVIR:Adverse event:1.11), (LERCANIDIPINE HYDROCHLORIDE:Adverse event:0.46), (LETROZOLE:Adverse event:1.33), (LEVALBUTEROL HYDROCHLORIDE:Adverse event:0.9), (LEVOLEUCOVORIN CALCIUM:Adverse event:0.26), (LITHIUM:Adverse event:0.39), (METAXALONE:Adverse event:0.48), (METFORMIN HYDROCHLORIDE\VILDAGLIPTIN:Adverse event:0.09), (METHENAMINE HIPPURATE:Adverse event:0.44), (METHENOLONE ENANTHATE:Adverse event:0.13), (METOLAZONE:Adverse event:0.32), (MIDODRINE:Adverse event:0.17), (MIZORIBINE:Adverse event:0.09), (MUPIROCIN:Adverse event:0.34), (MUPIROCIN\MUPIROCIN CALCIUM:Adverse event:0.2), (NADOLOL:Adverse event:0.14), (NADROPARIN CALCIUM:Adverse event:0.61), (NILOTINIB:Adverse event:2.41), (OCTREOTIDE ACETATE:Adverse event:0.12), (OLAPARIB:Adverse event:0.17), (OMEGA-3-ACID ETHYL ESTERS:Adverse event:0.85), (PAROXETINE:Adverse event:0.88), (PAROXETINE\PAROXETINE HYDROCHLORIDE:Adverse event:0.26), (PEGINTERFERON BETA-1A:Adverse event:0.08), (PEMETREXED:Adverse event:0.63), (PIROXICAM:Adverse event:0.25), (PONATINIB:Adverse event:0.91), (PRAMIPEXOLE:Adverse event:0.71), (PRIMIDONE:Adverse event:0.92), (PROGESTERONE:Adverse event:0.14), (PYRAZINAMIDE:Adverse event:0.72), (RADIATION THERAPY:Adverse event:0.16), (RALOXIFENE HYDROCHLORIDE:Adverse event:0.23), (RALTEGRAVIR POTASSIUM:Adverse event:0.49), (RAMUCIRUMAB:Adverse event:0.39), (REGORAFENIB:Adverse event:0.63), (RIVASTIGMINE:Adverse event:0.07), (RIZATRIPTAN BENZOATE:Adverse event:0.35), (ROFLUMILAST:Adverse event:0.5), (SACUBITRIL\VALSARTAN:Adverse event:0.47), (SECUKINUMAB:Adverse event:0.6), (SEVELAMER HYDROCHLORIDE:Adverse event:0.14), (SODIUM FERROUS CITRATE:Adverse event:0.11), (SODIUM OXYBATE:Adverse event:0.6), (SULFAMETHOXAZOLE:Adverse event:1.04), (SUMATRIPTAN:Adverse event:0.32), (SUMATRIPTAN\SUMATRIPTAN SUCCINATE:Adverse event:0.28), (SUNITINIB MALATE:Adverse event:0.07), (TERBUTALINE SULFATE:Adverse event:0.23), (TERIFLUNOMIDE:Adverse event:0.03), (TIMOLOL MALEATE:Adverse event:0.27), (TIOTROPIUM:Adverse event:0.55), (TRAMETINIB:Adverse event:0.04), (TROSPIUM CHLORIDE:Adverse event:0.29), (UMECLIDINIUM BROMIDE\VILANTEROL TRIFENATATE:Adverse event:0.34), (VITAMIN A:Adverse event:0.24), (ZONISAMIDE:Adverse event:0.37), (ALCOHOL:Affect lability:0.12), (AMISULPRIDE:Affect lability:0.03), (AMOXICILLIN:Affect lability:0), (CALCIUM CARBONATE\CHOLECALCIFEROL:Affect lability:0.09), (DEVICE:Affect lability:0.14), (DIGOXIN:Affect lability:0.47), (ESZOPICLONE:Affect lability:0.32), (EVOLOCUMAB:Affect lability:0.25), (FERROUS SULFATE:Affect lability:0.25), (GUANFACINE:Affect lability:0.09), (HYDROMORPHONE HYDROCHLORIDE:Affect lability:0.19), (INSULIN HUMAN:Affect lability:0.35), (NITROGLYCERIN:Affect lability:0.05), (PANTOPRAZOLE:Affect lability:0.47), (PAROXETINE:Affect lability:0.47), (PHENYTOIN:Affect lability:0.27), (POLYETHYLENE GLYCOLS:Affect lability:0.2), (PRAMIPEXOLE DIHYDROCHLORIDE:Affect lability:0.83), (PRAVASTATIN:Affect lability:0.46), (RAMIPRIL:Affect lability:0.36), (RANOLAZINE:Affect lability:0.44), (ROPINIROLE HYDROCHLORIDE:Affect lability:0.27), (SACUBITRIL\VALSARTAN:Affect lability:0.21), (TIMOLOL:Affect lability:0.1), (TRIAMCINOLONE ACETONIDE:Affect lability:0.02), (VARENICLINE TARTRATE:Affect lability:1.22), (. ALPHA.1-PROTEINASE INHIBITOR HUMAN:Affective disorder:0.36), (ACARBOSE:Affective disorder:0.34), (ACETAMINOPHEN\DICHLORALPHENAZONE\ISOMETHEPTENE:Affectiv e disorder:0.03), (ACETAMINOPHEN\OXYCODONE HYDROCHLORIDE\OXYCODONE TEREPHTHALATE:Affective disorder:0.73), (ALFACALCIDOL:Affective disorder:0.44), (AMILORIDE HYDROCHLORIDE\HYDROCHLOROTHIAZIDE:Affective disorder:0.07), (AMLODIPINE BESYLATE\HYDROCHLOROTHIAZIDE\OLMESARTAN MEDOXOMIL:Affective disorder:0.27), (AMLODIPINE\VALSARTAN:Affective disorder:0.23), (AMPHETAMINE SULFATE:Affective disorder:0.31), (AMPICILLIN:Affective disorder:0), (ANTIHISTAMINES NOS:Affective disorder:0.17), (APREPITANT:Affective disorder:0.03), (ARIPIPRAZOLE LAUROXIL:Affective disorder:1.37), (BENSERAZIDE\LEVODOPA:Affective disorder:0.6), (BETAMETHASONE DIPROPIONATE\CALCIPOTRIENE\CALCIPOTRIENE HYDRATE:Affective disorder:0.07), (BIFIDOBACTERIUM LONGUM SUBSP. INFANTIS:Affective disorder:0.68), (BRINZOLAMIDE:Affective disorder:0.23), (BRIVARACETAM:Affective disorder:0.35), (BROTIZOLAM:Affective disorder:0.36), (BUDESONIDE\FORMOTEROL FUMARATE:Affective disorder:0.33), (BUTYLSCOPOLAMINE BROMIDE:Affective disorder:0.52), (CALCIUM ACETATE:Affective disorder:1.11), (CALCIUM\MAGNESIUM:Affective disorder:0.39), (CANAGLIFLOZIN\METFORMIN HYDROCHLORIDE:Affective disorder:0.87), (CANAKINUMAB:Affective disorder:0.1), (CANNABIS SATIVA SUBSP. SATIVA FLOWERING TOP:Affective disorder:0.02), (CAPTOPRIL:Affective disorder:0.55), (CETIRIZINE HYDROCHLORIDE\PSEUDOEPHEDRINE HYDROCHLORIDE:Affective disorder:0.58), (CHLORPROTHIXENE:Affective disorder:0.33), (CHONDROITIN SULFATE (BOVINE)\GLUCOSAMINE HYDROCHLORIDE:Affective disorder:0.73), (CIMETIDINE:Affective disorder:0.98), (CINACALCET HYDROCHLORIDE:Affective disorder:0.35), (CLOMETHIAZOLE:Affective disorder:0.1), (CODEINE PHOSPHATE:Affective disorder:0.23), (DANAZOL:Affective disorder:0.16), (DAPAGLIFLOZIN:Affective disorder:0.04), (DASATINIB:Affective disorder:1.28), (DEFLAZACORT:Affective disorder:0.37), (DIAMORPHINE:Affective disorder:0.16), (DIPYRONE:Affective disorder:0.04), (DOFETILIDE:Affective disorder:0.78), (DRONEDARONE:Affective disorder:0.02), (DUTASTERIDE\TAMSULOSIN HYDROCHLORIDE:Affective disorder:0.37), (EDOXABAN TOSYLATE:Affective disorder:0.27), (ELETRIPTAN HYDROBROMIDE:Affective disorder:2.15), (EPLERENONE:Affective disorder:0.6), (ESTRADIOL\NORETHINDRONE ACETATE:Affective disorder:0.4), (ESTROGENS:Affective disorder:0.26), (ETHAMBUTOL HYDROCHLORIDE:Affective disorder:0.95), (ETORICOXIB:Affective disorder:0.2), (FLECAINIDE:Affective disorder:1.18), (FLUDROCORTISONE:Affective disorder:0.39), (FORMOTEROL FUMARATE\MOMETASONE FUROATE:Affective disorder:0.14), (FROVATRIPTAN SUCCINATE:Affective disorder:0.06), (GARLIC:Affective disorder:0.19), (GLYCOPYRRONIUM\INDACATEROL:Affective disorder:0.44), (HYDROCHLOROTHIAZIDE\IRBESARTAN:Affective disorder:0.77), (HYDROCHLOROTHIAZIDE\TELMISARTAN:Affective disorder:0.46), (HYDROXYCHLOROQUINE DIPHOSPHATE:Affective disorder:0.27), (HYPERICUM PERFORATUM:Affective disorder:0.38), (IMATINIB MESYLATE:Affective disorder:0.34), (INDAPAMIDE\PERINDOPRIL:Affective disorder:0.81), (INFLIXIMAB-DYYB:Affective disorder:0.01), (INSULIN DEGLUDEC:Affective disorder:0.44), (INTERFERON:Affective disorder:0.15), (INTERFERON ALFA-2B:Affective disorder:0.09), (ISTRADEFYLLINE:Affective disorder:0.27), (ITRACONAZOLE:Affective disorder:0.05), (LABETALOL HYDROCHLORIDE:Affective disorder:0.47), (LACTASE:Affective disorder:0.34), (LITHIUM SULFATE:Affective disorder:0.83), (LOMITAPIDE MESYLATE:Affective disorder:0.72), (LYSERGIDE:Affective disorder:0.18), (MEFENAMIC ACID:Affective disorder:0.08), (METHYLPREDNISOLONE ACETATE:Affective disorder:0.98), (MIDAZOLAM\MIDAZOLAM HYDROCHLORIDE:Affective disorder:0.02), (MIDODRINE HYDROCHLORIDE:Affective disorder:0.66), (MINOCYCLINE HYDROCHLORIDE:Affective disorder:0.31), (MORPHINE SULFATE\NALTREXONE HYDROCHLORIDE:Affective disorder:0.13), (MORPHINE\MORPHINE HYDROCHLORIDE\MORPHINE SULFATE\MORPHINE TARTRATE:Affective disorder:0.35), (MUPIROCIN\MUPIROCIN CALCIUM:Affective disorder:0.58), (NALOXONE:Affective disorder:0.14), (NILOTINIB:Affective disorder:1.05), (NINTEDANIB:Affective disorder:0.29), (NITROFURANTOIN\NITROFURANTOIN MONOHYDRATE:Affective disorder:0.69), (NORETHINDRONE ACETATE:Affective disorder:0.15), (OLODATEROL HYDROCHLORIDE\TIOTROPIUM BROMIDE MONOHYDRATE:Affective disorder:0.11), (ORPHENADRINE:Affective disorder:0.24), (OXYBUTYNIN\OXYBUTYNIN CHLORIDE:Affective disorder:0.01), (PATIROMER:Affective disorder:0.15), (PEGFILGRASTIM:Affective disorder:0.33), (PEGINTERFERON BETA-1A: Affective disorder:0.82), (PEROSPIRONE:Affective disorder:0.19), (PHENTERMINE HYDROCHLORIDE:Affective disorder:0.36), (PONATINIB:Affective disorder:0.64), (POSACONAZOLE:Affective disorder:0.08), (PREDNISOLONE ACETATE:Affective disorder:0.13), (PREDNISOLONE SODIUM PHOSPHATE:Affective disorder:0.42), (PREDNISOLONE\PREDNISOLONE ACETATE\PREDNISOLONE SODIUM SUCCINATE:Affective disorder:0.11), (PROTRIPTYLINE HYDROCHLORIDE:Affective disorder:0.52), (PSEUDOEPHEDRINE HYDROCHLORIDE:Affective disorder:0.34), (RASAGILINE:Affective disorder:0.5), (SALMETEROL XINAFOATE:Affective disorder:0.21), (SERTINDOLE:Affective disorder:0.41), (TERBINAFINE HYDROCHLORIDE:Affective disorder:0.72), (TERBUTALINE SULFATE:Affective disorder:1.09), (TIAPRIDE:Affective disorder:0.24), (TOLTERODINE:Affective disorder:0.18), (ZINC:Affective disorder:0.91), (ZUCLOPENTHIXOL HYDROCHLORIDE:Affective disorder:0.01), (ACARBOSE:Age-related macular degeneration:0.15), (ALENDRONIC ACID:Age-related macular degeneration:0.01), (BISACODYL:Age-related macular degeneration:0.03), (BISACODYL OR DOCUSATE SODIUM:Age-related macular degeneration:0.27), (BUDESONIDE\FORMOTEROL FUMARATE:Age-related macular degeneration:0.22), (CALCITRIOL:Age-related macular degeneration:0.68), (CARFILZOMIB:Age-related macular degeneration:0.04), (CARVEDILOL PHOSPHATE:Age-related macular degeneration:0.12), (CERTOLIZUMAB PEGOL:Age-related macular degeneration:0.51), (CHOLESTYRAMINE:Age-related macular degeneration:0.26), (CLOTRIMAZOLE:Age-related macular degeneration:0.21), (DASATINIB:Age-related macular degeneration:0.23), (DEVICE:Age-related macular degeneration:0.07), (DIPHENHYDRAMINE:Age-related macular degeneration:0.24), (DOFETILIDE:Age-related macular degeneration:0.13), (ENOXAPARIN:Age-related macular degeneration:0.42), (ESZOPICLONE:Age-related macular degeneration:0.23), (EVEROLIMUS:Age-related macular degeneration:0.9), (EVOLOCUMAB:Age-related macular degeneration:0.63), (FERROUS SULFATE:Age-related macular degeneration:0.74), (FESOTERODINE FUMARATE:Age-related macular degeneration:0.29), (FLUOCINOLONE ACETONIDE:Age-related macular degeneration:0.17), (FLUTICASONE FUROATE\VILANTEROL TRIFENATATE:Age-related macular degeneration:0.2), (GLYCOPYRRONIUM\INDACATEROL:Age-related macular degeneration:0.42), (HEPARIN SODIUM:Age-related macular degeneration:0.15), (HYDROCHLOROTHIAZIDE\LISINOPRIL:Age-related macular degeneration:0.37), (IBRUTINIB:Age-related macular degeneration:0.59), (ISOSORBIDE:Age-related macular degeneration:0.61), (LEDIPASVIR\SOFOSBUVIR:Age-related macular degeneration:0.13), (LEVALBUTEROL HYDROCHLORIDE:Age-related macular degeneration:0.07), (METHYLPREDNISOLONE SODIUM SUCCINATE:Age-related macular degeneration:0.54), (MOXIFLOXACIN:Age-related macular degeneration:0.07), (NALOXONE HYDROCHLORIDE\OXYCODONE HYDROCHLORIDE:Age-related macular degeneration:0.49), (NILOTINIB:Age-related macular degeneration:0.14), (NITROFURANTOIN:Age-related macular degeneration:0.46), (OLODATEROL HYDROCHLORIDE\TIOTROPIUM BROMIDE MONOHYDRATE:Age-related macular degeneration:0.23), (OXYGEN:Age-related macular degeneration:0.52), (PAZOPANIB HYDROCHLORIDE:Age-related macular degeneration:0.18), (PENTOXIFYLLINE:Age-related macular degeneration:0.05), (PHENYLEPHRINE HYDROCHLORIDE\TROPICAMIDE:Age-related macular degeneration:0.68), (PRAMIPEXOLE:Age-related macular degeneration:0.49), (ROFLUMILAST:Age-related macular degeneration:0.08), (ROPINIROLE HYDROCHLORIDE:Age-related macular degeneration:0.06), (SACUBITRIL\VALSARTAN:Age-related macular degeneration:0.35), (SECUKINUMAB:Age-related macular degeneration:0.45), (TIMOLOL\TRAVOPROST:Age-related macular degeneration:0.63), (TOPIRAMATE:Age-related macular degeneration:0.15), (UMECLIDINIUM BROMIDE\VILANTEROL TRIFENATATE:Age-related macular degeneration:0.13), (VALACYCLOVIR HYDROCHLORIDE:Age-related macular degeneration:0.05), (ARIPIPRAZOLE LAUROXIL:Aggression:0.54),(ARMODAFINIL:Aggression:0.25), (ATOMOXETINE:Aggression: 0.08), (BUPRENORPHINE HYDROCHLORIDE:Aggression:0.58), (BUPRENORPHINE HYDROCHLORIDE\NALOXONE HYDROCHLORIDE:Aggression:0.15), (CERTOLIZUMAB PEGOL:Aggression:0.08), (CHOLESTYRAMINE:Aggression:0.08), (CYCLOBENZAPRINE: Aggression:0.25), (DILTIAZEM:Aggression:0.18), (ENOXAPARIN:Aggression:0.33), (EVOLOC UMAB:Aggression:0.31), (FENOFIBRATE:Aggression:0.02), (IBRUTINIB:Aggression:0.2), (L OVASTATIN:Aggression:0.37), (METHYLPREDNISOLONE SODIUM SUCCINATE:Aggression:0.06), (OXYCODONE:Aggression:0.08), (POLYETHYLENE GLYCOLS:Aggression:0.24), (PRIMIDONE:Aggression:0.25), (RANOLAZINE:Aggression:0.15), (SCOPOLAMINE:Aggression:0.09), (TERIFLUNOMIDE:Aggression:0.01), (TIZANIDINE HYDROCHLORIDE:Aggression:0.19), (TRIHEXYPHENIDYL:Aggression:0.15), (VARENICLI NE TARTRATE:Aggression:1.18), (VERAPAMIL HYDROCHLORIDE:Aggression:0.35), (.ALPHA.1-PROTEINASE INHIBITOR HUMAN:Agitation:0.19), (ABACAVIR SULFATE\DOLUTEGRAVIR SODIUM\LAMIVUDINE:Agitation:0.17), (ACETAMINOPHEN\OXYCODONE\OXYCODON E HYDROCHLORIDE:Agitation:0.81), (ADO-TRASTUZUMAB EMTANSINE:Agitation:0.19), (AGALSIDASE BETA:Agitation:0.5), (ALEMTUZUMAB:Agitation:0.02), (ALENDRONIC ACID:Agitation:1.02), (ALFACALCIDOL:Agitation:0.34), (AMIKACIN:Agitation:0.53), (AMO BARBITAL SODIUM:Agitation:0.04), (ASENAPINE:Agitation:0.37), (ATRACURIUM BESYLATE:Agitation:0.24), (AZACITIDINE:Agitation:0.11), (BENPERIDOL:Agitation:0.03), (BLONANSERIN:Agitation:0.4), (CALCITONIN SALMON:Agitation:0.33), (CANAGLIFLOZIN:Agitation:0.03), (CANAKINUMAB:Agitation:0.14), (CANDESARTAN:Agitation:1.63), (CANNABIS SATIVA SUBSP. SATIVA FLOWERING TOP:Agitation:0.02), (CARBIDOPA:Agitation:0.46), (CARBIDOPA\MELEVODOPA:Agitation: 0.16), (CARVEDILOL PHOSPHATE:Agitation:0.75), (CEFEPIME\CEFEPIME HYDROCHLORIDE:Agitation:0.2), (CEFOTAXIME\CEFOTAXIME SODIUM:Agitation:0.05), (CERTOLIZUMAB PEGOL:Agitation:0.95), (CHLORAMBUCIL:Agitation:0.08), (CHOLESTYRAMINE:Agitation: 1.11), (CHONDROITIN SULFATE (BOVINE)\GLUCOSAMINE HYDROCHLORIDE:Agitation:0.11), (CICLESONIDE:Agitation:0.4), (CIMETIDINE:Agitation: 0.68), (CINACALCET HYDROCHLORIDE:Agitation:0.05), (CISAPRIDE:Agitation:0.04), (CLINDAMYCIN:Agitation:0.79), (COLISTIMETHATE SODIUM:Agitation:0.47), (CORTICOTROPIN:Agitation:0.12), (CORTISONE\HYDROCORTIS ONE:Agitation:0.78), (DABIGATRAN:Agitation:0.63), (DANAZOL:Agitation:0.97), (DASATIN 1B:Agitation:1.44), (DEFLAZACORT:Agitation:0.41), (DEVICE:Agitation:0.13), (DIPYRIDAM OLE:Agitation:0.03), (DOBUTAMINE HYDROCHLORIDE:Agitation:0.33), (DOCUSATE CALCIUM:Agitation:0.5), (DOFETILIDE:Agitation:0.43), (ELETRIPTAN HYDROBROMIDE:Agitation:1.25), (ESLICARBAZEPINE:Agitation:0.06), (ESMOLOL HYDROCHLORIDE:Agitation:0.87), (ESTROGENS, CONJUGATED\MEDROXYPROGESTERONE ACETATE:Agitation:0.56), (ETORICOXIB:Agitation:0.62), (EVOLOCUMAB:Agitation:0.3), (E XEMESTANE:Agitation:0.09), (FENOTEROL HYDROBROMIDE\IPRATROPIUM BROMIDE:Agitation:0.24), (FERROUS SULFATE\GLYCINE:Agitation:0.33), (FESOTERODINE FUMARATE:Agitation:1.16), (FLUDROCORTISONE ACETATE:Agitation:0.85), (FLUPHENAZINE HYDROCHLORIDE:Agitation:0.08), (FLUTICASONE FUROATE:Agitation:0.5), (FLUVASTATIN SODIUM:Agitation:0.2), (FLUVOXAMINE MALEATE:Agitation:0.84), (FORMOTEROL:Agitation:0.67), (FORMOTEROL FUMARATE\MOMETASONE FUROATE:Agitation:0.15), (FOSFOMYCIN:Agitation:0.22), (HYPERICUM PERFORATUM:Agitation:0.17), (ILOPROST:Agitation:0.26), (INDAPAMIDE\PERINDOPRIL: Agitation:1.35), (INVESTIGATIONAL PRODUCT:Agitation:0.24), (ISOFLURANE:Agitation:0.94), (LEDIPASVIR\SOFOSBUVIR: Agitation:0.84), (LEVALBUTEROL:Agitation:0.06), (LISDEXAMFETAMINE:Agitation:0.36), (M ECLIZINE\MECLIZINE HYDROCHLORIDE:Agitation:0.02), (METHENAMINE HIPPURATE:Agitation:1.04), (METHYLENEDIOXYMETHAMPHETAMINE:Agitation:0.04), (METHYLPREDNISOLONE ACETATE:Agitation:1.41), (MIDODRINE HYDROCHLORIDE:Agitation:0.29), (MINOCYCLINE\MINOCYCLINE HYDROCHLORIDE:Agitation:0.98), (MOXIFLOXACIN:Agitation:0.81), (MOXIFLOXACIN HYDROCHLORIDE:Agitation:1.99), (MOXONIDINE:Agitation:0.58), (MUPIROCIN\MUPIRO CIN CALCIUM:Agitation:0.51), (NABUMETONE:Agitation:0.29), (NADOLOL:Agitation:0.09), (NA DROPARIN CALCIUM:Agitation:0.83), (NALTREXONE:Agitation:0.69), (NATEGLINIDE:Agitation:0.03), (NILOTINIB:Agitation:1.68), (NORTRIPTYLINE:Agitation:1.13), (OMEGA-3-ACID ETHYL ESTERS:Agitation:1.04), (OSELTAMIVIR:Agitation:0.82), (OXYMORPHONE:Agitation:0.66), (PANCRELIPASE:Agitation:0.47), (PANTOPRAZOLE MAGNESIUM:Agitation:1.03), (PEGFILGRASTIM:Agitation:0.6), (PEGINTERFERON BETA-1A:Agitation:0.27), (PENICILLIN V:Agitation:0.02), (PEROSPIRONE:Agitation:0.55), (PHENPROCOUMON:Agitation:0.18), (PI RIBEDIL:Agitation:0.61), (PIROXICAM:Agitation:0.16), (PITAVASTATIN CALCIUM:Agitation:0.39), (PLANTAGO SEED:Agitation:0.17), (POSACONAZOLE:Agitation:1.15), (PREDNISOLONE SODIUM PHOSPHATE:Agitation:0.21), (PREDNISOLONE\PREDNISOLONE ACETATE\PREDNISOLONE SODIUM SUCCINATE:Agitation:0.41), (PROCYCLIDINE HYDROCHLORIDE:Agitation:0.08), (PSYLLIUM HUSK:Agitation:0.04), (RANOLAZINE:Agitation:1.38), (RASAGILINE:Agitation:0.42), (RETI NOL:Agitation:0.06), (ROPIVACAINE:Agitation:0.32), (SACCHAROMYCES CEREVISIAE:Agitation:0.02), (SAFINAMIDE:Agitation:0.17), (SECUKINUMAB:Agitation:0.53), (SELEGILINE:Agitation:0.98), (SERTINDOLE:Agitation:0.16), (SILODOSIN:Agitation:0.38), (SUCCINYLCHOLINE CHLORIDE:Agitation:0.83), (SUCRALFATE:Agitation:0.92), (SUGAMMADEX:Agitation:0.09), (SULFAMETHOXAZOLE:Agitation:0.73), (SULPIRIDE:Agitation:0.02), (SUVOREXANT: Agitation:0.3), (THALIDOMIDE:Agitation:1.16), (THIOPENTAL:Agitation:0.47), (THYROID, PORCINE:Agitation:0.01), (TIGECYCLINE:Agitation:0.37), (TIMOLOL:Agitation:0.65), (TOC OPHEROL:Agitation:0.05), (TOLCAPONE:Agitation:0.04), (TRIAMCINOLONE:Agitation:0.13), (TRIMIPRAMINE MALEATE:Agitation:0.72), (TROPINE BENZYLATE:Agitation:0.32), (TROSPIUM CHLORIDE:Agitation:0.72), (UMECLIDINIUM BROMIDE\VILANTEROL TRIFENATATE:Agitation:1.01), (VALGANCICLOVIR HYDROCHLORIDE:Agitation:0.98), (VINCRISTINE SULFATE:Agitation:0.49),(ARMODAFINIL:Alcoholism:0.39), (CERTOLIZUMAB PEGOL:Alcoholism:0.6), (CHOLESTYRAMINE:Alcoholism:0.55), (CIPROFLOXACIN:Alcoholism: 0.12), (DALFAMPRIDINE:Alcoholism:0.51), (DIGOXIN:Alcoholism:0.01), (DILTIAZEM: Alcoholism:0.03), (DIPHENHYDRAMINE:Alcoholism:0.06), (ENALAPRIL MALEATE:Alcoholism:0.07), (EVOLOCUMAB:Alcoholism:0.9), (FERROUS SULFATE\FERROUS SULFATE, DRIED:Alcoholism:0.59), (GUANFACINE:Alcoholism:0.02), (INDOMETHACIN:Alcoholism:00.23), (INSULIN DETEMIR:Alcoholism:0.17), (LEDIPASVIR\SOFOSBUVIR:Alcoholism:0.66), (LOVASTATIN:Alcoholism:0.5), (METAXALONE:Alcoholism:0.03), (METHYLPREDNISOLONE SODIUM SUCCINATE:Alcoholism:0.25), (METRONIDAZOLE\METRONIDAZOLE HYDROCHLORIDE:Alcoholism:0.02), (MODAFINIL:Alcoholism:0.12), (POLYETHYLENE GLYCOLS:Alcoholism:0.38), (PRAMIPEXOLE:Alcoholism:0.21), (PRAVASTATIN SODIUM:Alcoholism:0.65), (RANOLAZINE:Alcoholism:0.06), (SECUKINUMAB:Alcoholism: 0.02), (SUMATRIPTAN\SUMATRIPTAN SUCCINATE:Alcoholism:0.12), (TELMISARTAN:Alcoholism:0), (UMECLIDINIUM BROMIDE\VILANTEROL TRIFENATATE:Alcoholism:0.04), (WARFARIN:Alcoholism:0.44), (WARFARIN SODIUM:Alcoholism:0.14), (AMPHETAMINE ASPARTATE\AMPHETAMINE SULFATE\DEXTROAMPHETAMINE SACCHARATE\DEXTROAMPHETAMINE SULFATE:Allergic sinusitis:0.01), (DEXLANSOPRAZOLE:Allergic sinusitis:0.25), (DIPHENHYDRAMINE:Allergic sinusitis:0.26), (EVOLOCUMAB:Allergic sinusitis:0.25), (MELATONIN:Allergic sinusitis:0.01), (PRAVASTATIN SODIUM:Allergic sinusitis:0.21), (TERIFLUNOMIDE:Allergic sinusitis:0.22), (ADO-TRASTUZUMAB EMTANSINE:Allergy prophylaxis:0.2), (ALBUTEROL SULFATE\IPRATROPIUM BROMIDE:Allergy prophylaxis:0.32), (ALENDRONIC ACID:Allergy prophylaxis:0.31), (AMIODARONE:Allergy prophylaxis:0.79), (BENAZEPRIL HYDROCHLORIDE:Allergy prophylaxis:0.11), (BLINATUMOMAB:Allergy prophylaxis:0.51), (BUPROPION:Allergy prophylaxis:0.04), (CALCITONIN SALMON:Allergy prophylaxis:0.12), (CANAKINUMAB:Allergy prophylaxis:0.16), (CANDESARTAN CILEXETIL\HYDROCHLOROTHIAZIDE:Allergy prophylaxis:0.33), (CERTOLIZUMAB PEGOL:Allergy prophylaxis:1.06), (CHLORAMBUCIL:Allergy prophylaxis:0.25), (CHOLESTYRAMINE:Allergy prophylaxis:0.26), (CLOTRIMAZOLE:Allergy prophylaxis:0.29), (CLOZAPINE:Allergy prophylaxis:0.1), (CORTISONE\HYDROCORTISONE:Allergy prophylaxis:0.61), (CRIZOTINIB:Allergy prophylaxis:0.2), (DEXLANSOPRAZOLE:Allergy prophylaxis:0.78), (DIPYRONE:Allergy prophylaxis:0.04), (DOCUSATE:Allergy prophylaxis:0.62), (DUTASTERIDE:Allergy prophylaxis:0.08), (ELOTUZUMAB:Allergy prophylaxis:0.2), (ENALAPRIL:Allergy prophylaxis:1.76), (ENALAPRIL MALEATE:Allergy prophylaxis:0.15), (EVEROLIMUS:Allergy prophylaxis:0.61), (EVOLOCUMAB:Allergy prophylaxis:0.06), (FENOFIBRATE:Allergy prophylaxis:0.41), (FERROUS SULFATE\FERROUS SULFATE, DRIED:Allergy prophylaxis:1.13), (FINGOLIMOD HYDROCHLORIDE:Allergy prophylaxis:0.27), (FLECAINIDE:Allergy prophylaxis:0.24), (FLUTICASONE FUROATE\SALMETEROL XINAFOATE:Allergy prophylaxis:0.07), (GIMERACIL\OTERACIL\TEGAFUR:Allergy prophylaxis:0), (HYDROCHLOROTHIAZIDE\IRBESARTAN:Allergy prophylaxis:0), (HYDROCHLOROTHIAZIDE\LISINOPRIL:Allergy prophylaxis:0.68), (HYDROXYCHLOROQUINE DIPHOSPHATE:Allergy prophylaxis:0.21), (IBRITUMOMAB TIUXETAN:Allergy prophylaxis:0.39), (IMATINIB MESYLATE:Allergy prophylaxis:0.83), (IMMUNE GLOBULIN NOS:Allergy prophylaxis:0.33), (INSULIN DETEMIR:Allergy prophylaxis:0.14), (IPRATROPIUM:Allergy prophylaxis:0.26), (ISOSORBIDE MONONITRATE:Allergy prophylaxis:0.5), (LATANOPROST:Allergy prophylaxis:0.17), (LEDIPASVIR\SOFOSBUVIR:Allergy prophylaxis:0.13), (LERCANIDIPINE HYDROCHLORIDE:Allergy prophylaxis:0.06), (LOVASTATIN:Allergy prophylaxis:0.4), (MOXIFLOXACIN:Allergy prophylaxis:0.24), (OXYGEN:Allergy prophylaxis:0.56), (PANTOPRAZOLE MAGNESIUM:Allergy prophylaxis:0.15), (PENICILLIN:Allergy prophylaxis:0.21), (PERINDOPRIL:Allergy prophylaxis:0.34), (RABEPRAZOLE:Allergy prophylaxis:0.1), (REGORAFENIB:Allergy prophylaxis:0.43), (ROFLUMILAST:Allergy prophylaxis:0.03), (SECUKINUMAB:Allergy prophylaxis:0.23), (TERIFLUNOMIDE:Allergy prophylaxis:0.12), (TIZANIDINE HYDROCHLORIDE:Allergy prophylaxis:0.15), (TOCILIZUMAB:Allergy prophylaxis:0.46), (VENLAFAXINE HYDROCHLORIDE:Allergy prophylaxis:0.57), (VITAMIN B:Allergy prophylaxis:0.03), (EVOLOCUMAB:Allergy to animal:0.12), (.ALPHA.1-PROTEINASE INHIBITOR HUMAN:Alopecia:0.53), (ACARBOSE:Alopecia:0.32), (ACETAMINOPHEN\PROPOXYPHEN E HYDROCHLORIDE:Alopecia:0.2), (ACETAMINOPHEN\TRAMADOL:Alopecia:0.17), (AMA NTADINE HYDROCHLORIDE:Alopecia:0.25), (AMLODIPINE BESYLATE\HYDROCHLOROTHIAZIDE\OLMESARTAN MEDOXOMIL:Alopecia:0.29), (AMLODIPINE\VALSARTAN:Alopecia:0.05), (AMMONIUM LACTATE:Alopecia:0.5), (ASPIRIN\BUTALBITAL\CAFFEINE:Alopecia:0.02), (BETAMETH ASONE\CALCIPOTRIENE:Alopecia:0.08), (BRIMONIDINE TARTRATE\TIMOLOL MALEATE:Alopecia:0.1), (BROMAZEPAM:Alopecia:0.05), (BUPRENORPHINE HYDROCHLORIDE:Alopecia:0.62), (CALCIUM ACETATE:Alopecia:0.84), (CANAGLIFLOZIN\METFORMIN HYDROCHLORIDE:Alopecia:0.68), (CANAKINUMAB:Alopecia:0.03), (CANDESARTAN CILEXETIL\HYDROCHLOROTHIAZIDE:Alopecia:0.81), (CAPSAICIN:Alopecia:0.42), (CAR VEDILOL PHOSPHATE:Alopecia:1.27), (CEVIMELINE HYDROCHLORIDE:Alopecia:0.14), (CHLORDIAZEPOXIDE HYDROCHLORIDE\CLIDINIUM BROMIDE:Alopecia:0.46), (CLARITHROMYCIN:Alopecia:0.02), (CLOZAPINE:Alopecia:0.71), (DEFLAZACORT:Alopecia:0.02), (DOFETILIDE:Alopecia:0.63), (DORZOLAMIDE:Alopecia:0.15), (DOXAZOSIN MESYLATE:Alopecia:0.74), (DUTASTERIDE\TAMSULOSIN HYDROCHLORIDE:Alopecia:0.05), (EDOXABAN TOSYLATE:Alopecia:0.52), (ELETRIPTAN HYDROBROMIDE:Alopecia:1.96), (EMPAGLIFLOZIN\LINAGLIPTIN:Alopecia:0.74), (ERY THROMYCIN:Alopecia:0.55), (ERYTHROPOIETIN:Alopecia:0.51), (ESOMEPRAZOLE MAGNESIUM\NAPROXEN:Alopecia:0.54), (ETHINYL ESTRADIOL\NORETHINDRONE:Alopecia:0.1), (ETORICOXIB:Alopecia:0.43), (FLUTICAS ONE FUROATE:Alopecia:0.64), (FLUTICASONE FUROATE\SALMETEROL XINAFOATE:Alopecia:0.08), (FORMOTEROL FUMARATE:Alopecia:0.18), (FUSIDIC ACID:Alopecia:0.17), (GLYCOPYRRONIUM\INDACATEROL:Alopecia:0.18), (GUANFACIN E:Alopecia:0.5), (GUANFACINE HYDROCHLORIDE:Alopecia:0.07), (HYDROCHLOROTHIAZIDE\IRBESARTAN:Alopecia: 0.47), (HYDROCHLOROTHIAZIDE\TELMISARTAN:Alopecia:0.4), (HYDROCORTISONE ACETATE:Alopecia:0.23), (IBRUTINIB:Alopecia:0.49), (INSULIN DEGLUDEC:Alopecia:0.28), (IPRATROPIUM:Alopecia:0.87), (LERCANIDIPINE:Alopecia:0.3), (LERCANIDIPINE HYDROCHLORIDE:Alopecia:0.31), (LEVOMILNACIPRAN HYDROCHLORIDE:Alopecia:0.33), (LIDOCAINE HYDROCHLORIDE:Alopecia:0.63), (LIDOCAINE\PRILOCAINE:Alopecia:0.27), (LOMITAPI DE MESYLATE:Alopecia:0.71), (MEPOLIZUMAB:Alopecia:0.16), (METHYLPREDNISOLONE ACETATE:Alopecia:1.01), (MIDODRINE HYDROCHLORIDE:Alopecia:0.13), (MUPIROCIN\MUPIROCIN CALCIUM:Alopecia:0.49), (NARATRIPTAN HYDROCHLORIDE:Alopecia:0.02), (PALIPERIDONE PALMITATE:Alopecia:0.17), (PAZOPANIB HYDROCHLORIDE:Alopecia:1.01), (PEGFILGRASTIM:Alopecia:0.02), (PEGINTERFERON BETA-1A:Alopecia:0.83), (PILOCARPINE HYDROCHLORIDE:Alopecia:0.04), (PRAZOSIN:Alopecia:0.71), (PRAZOSIN HYDROCHLORIDE:Alopecia:0.36), (PREDNISOLONE ACETATE:Alopecia:0.86), (PROTRIPTYLINE HYDROCHLORIDE:Alopecia:0.57), (PYRIDOSTIGMINE BROMIDE:Alopecia:0.01), (ROFECOXIB:Alopecia:0), (SACCHAROMYCES CEREVISIAE:Alopecia:0.13), (SCOPOLAMINE:Alopecia:0.95), (SELEGILINE:Alopecia:0.4), (STARCH, WHEAT:Alopecia:0.01), (SULFAMETHOXAZOLE:Alopecia:0.57), (SUMATRIPTAN\SUMAT RIPTAN SUCCINATE:Alopecia:2.53), (TERBUTALINE SULFATE:Alopecia:0.79), (TIOTROPIUM:Alopecia:0.01), (TROSPIUM CHLORIDE:Alopecia:0), (ZAFIRLUKAST:Alopecia:0.4), (ACETAMINOPHEN\OXYCODON E\OXYCODONE HYDROCHLORIDE:Alpha-1 anti-trypsin deficiency:0.48), (ALBIGLUTIDE:Alpha-1 anti-trypsin deficiency:0.47), (ALUMINUM HYDROXIDE\DIMETHICONE\MAGNESIUM HYDROXIDE:Alpha-1 anti-trypsin deficiency:0.03), (AMIODARONE HYDROCHLORIDE:Alpha-1 anti-trypsin deficiency:0.55), (AMISULPRIDE:Alpha-1 anti-trypsin deficiency:0.1), (AMITRIPTYLINE HYDROCHLORIDE:Alpha-1 anti-trypsin deficiency:0.61), (AMLODIPINE BESYLATE\HYDROCHLOROTHIAZIDE\OLMESARTAN MEDOXOMIL:Alpha-1 anti-trypsin deficiency:0.14), (AMLODIPINE\VALSARTAN:Alpha-1 anti-trypsin deficiency:0.05), (ANASTROZOLE:Alpha-1 anti-trypsin deficiency:0.95), (BENAZEPRIL\BENAZEPRIL HYDROCHLORIDE:Alpha-1 anti-trypsin deficiency:0.4), (BRINZOLAMIDE:Alpha-1 anti-trypsin deficiency:0.53), (BUDESONIDE\FORMOTEROL FUMARATE:Alpha-1 anti-trypsin deficiency:0.86), (BUPRENORPHINE HYDROCHLORIDE:Alpha-1 anti-trypsin deficiency:0.51), (CALCIUM CARBONATE\VITAMIN D:Alpha-1 anti-trypsin deficiency:0.39), (CANAGLIFLOZIN\METFORMIN HYDROCHLORIDE:Alpha-1 anti-trypsin deficiency:0.21), (CANAKINUMAB:Alpha-1 anti-trypsin deficiency:0.23), (CANDESARTAN:Alpha-1 anti-trypsin deficiency:0.63), (CANDESARTAN CILEXETIL\HYDROCHLOROTHIAZIDE:Alpha-1 anti-trypsin deficiency:0.41), (CAPTOPRIL:Alpha-1 anti-trypsin deficiency:0.59), (CARFILZOMIB:Alpha-1 anti-trypsin deficiency:0.25), (CERTOLIZUMAB PEGOL:Alpha-1 anti-trypsin deficiency:0.9), (CLINDAMYCIN:Alpha-1 anti-trypsin deficiency:0.48), (CORTICOTROPIN:Alpha-1 anti-trypsin deficiency:0.38), (CYPROHEPTADINE:Alpha-1 anti-trypsin deficiency:0.04), (DACLIZUMAB:Alpha-1 anti-trypsin deficiency:0.52), (DEXTROAMPHETAMINE SULFATE:Alpha-1 anti-trypsin deficiency:0.11), (EMPAGLIFLOZIN\LINAGLIPTIN:Alpha-1 anti-trypsin deficiency:0.07), (ENALAPRIL MALEATE:Alpha-1 anti-trypsin deficiency:0.89), (ENOXAPARIN:Alpha-1 anti-trypsin deficiency:1.24), (EPLERENONE:Alpha-1 anti-trypsin deficiency:0.24), (EVOLOCUMAB:Alpha-1 anti-trypsin deficiency:1.25), (FESOTERODINE FUMARATE:Alpha-1 anti-trypsin deficiency:0.88), (FLUVOXAMINE MALEATE:Alpha-1 anti-trypsin deficiency:0.05), (FORMOTEROL FUMARATE\MOMETASONE FUROATE:Alpha-1 anti-trypsin deficiency:1.18), (GLICLAZIDE:Alpha-1 anti-trypsin deficiency:0.15), (GLYCOPYRROLATE:Alpha-1 anti-trypsin deficiency:0.43), (GLYCOPYRRONIUM\INDACATEROL:Alpha-1 anti-trypsin deficiency:0.57), (HYDROCHLOROTHIAZIDE\IRBESARTAN:Alpha-1 anti-trypsin deficiency:0.45), (HYDROCODONE BITARTRATE\IBUPROFEN:Alpha-1 anti-trypsin deficiency:0.03), (HYDROXYUREA:Alpha-1 anti-trypsin deficiency:0.06), (IBRUTINIB:Alpha-1 anti-trypsin deficiency:0.37), (ILOPROST:Alpha-1 anti-trypsin deficiency:0.82), (IMIPRAMINE HYDROCHLORIDE:Alpha-1 anti-trypsin deficiency:0.5), (INDAPAMIDE:Alpha-1 anti-trypsin deficiency:0.14), (INSULIN GLULISINE:Alpha-1 anti-trypsin deficiency:0.12), (LACTASE:Alpha-1 anti-trypsin deficiency:0.29), (LEDIPASVIR\SOFOSBUVIR:Alpha-1 anti-trypsin deficiency:0.01), (LENALIDOMIDE:Alpha-1 anti-trypsin deficiency:0.4), (LITHIUM CARBONATE:Alpha-1 anti-trypsin deficiency:0.77), (LOMITAPIDE MESYLATE:Alpha-1 anti-trypsin deficiency:0.21), (METAXALONE:Alpha-1 anti-trypsin deficiency:1.8), (METHENAMINE HIPPURATE:Alpha-1 anti-trypsin deficiency:0.69), (METHYLPREDNISOLONE ACETATE:Alpha-1 anti-trypsin deficiency:0.85), (MIDODRINE HYDROCHLORIDE:Alpha-1 anti-trypsin deficiency:0.03), (MINOCYCLINE\MINOCYCLINE HYDROCHLORIDE:Alpha-1 anti-trypsin deficiency:0.29), (NALOXONE HYDROCHLORIDE\OXYCODONE HYDROCHLORIDE:Alpha-1 anti-trypsin deficiency:0.31), (NATEGLINIDE:Alpha-1 anti-trypsin deficiency:0.14), (NILOTINIB:Alpha-1 anti-trypsin deficiency:0.11), (NINTEDANIB:Alpha-1 anti-trypsin deficiency:0.45), (NORTRIPTYLINE HYDROCHLORIDE:Alpha-1 anti-trypsin deficiency:0.23), (OLOPATADINE:Alpha-1 anti-trypsin deficiency:0.08), (PANTOPRAZOLE MAGNESIUM:Alpha-1 anti-trypsin deficiency:1.65), (PEGINTERFERON BETA-1A:Alpha-1 anti-trypsin deficiency:0.06), (PENTOSAN POLYSULFATE SODIUM:Alpha-1 anti-trypsin deficiency:0.21), (PIMECROLIMUS:Alpha-1 anti-trypsin deficiency:0.09), (PONATINIB:Alpha-1 anti-trypsin deficiency:0.42), (PYRAZINAMIDE:Alpha-1 anti-trypsin deficiency:0.64), (QUETIAPINE:Alpha-1 anti-trypsin deficiency:0.79), (SCOPOLAMINE:Alpha-1 anti-trypsin deficiency:0.18), (SECUKINUMAB:Alpha-1 anti-trypsin deficiency:0.03), (SELEXIPAG:Alpha-1 anti-trypsin deficiency:0.3), (SODIUM OXYBATE:Alpha-1 anti-trypsin deficiency:1.82), (TERBUTALINE SULFATE:Alpha-1 anti-trypsin deficiency:0.46), (TETRACYCLINE:Alpha-1 anti-trypsin deficiency:0.61), (THYROID, PORCINE\THYROID, UNSPECIFIED:Alpha-1 anti-trypsin deficiency:0.07), (TICAGRELOR:Alpha-1 anti-trypsin deficiency:0.06), (TIMOLOL:Alpha-1 anti-trypsin deficiency:0.92), (TIMOLOL MALEATE:Alpha-1 anti-trypsin deficiency:0.38), (VALGANCICLOVIR HYDROCHLORIDE:Alpha-1 anti-trypsin deficiency:0.58), (VANCOMYCIN HYDROCHLORIDE:Alpha-1 anti-trypsin deficiency:0.13), (ZILEUTON:Alpha-1 anti-trypsin deficiency:0.29), (ZINC:Alpha-1 anti-trypsin deficiency:0.76), (ZOPICLONE:Alpha-1 anti-trypsin deficiency:0.53), (CALCIUM ACETATE:Amnesia:0.13), (CALCIUM CARBONATE\CHOLECALCIFEROL:Amnesia:0.39), (CARVEDILOL PHOSPHATE:Amnesia:0.04), (CEPHALEXIN:Amnesia:0.05), (CERTOLIZUMAB PEGOL:Amnesia:0.44), (CHOLESTYRAMINE:Amnesia:0.17), (CIPROFLOXACIN HYDROCHLORIDE:Amnesia:0), (CLONIDINE:Amnesia:0.42), (DASATINIB:Amnesia:0.12), (EVEROLIMUS:Amnesia:0.23), (EVOLOCUMAB:Amnesia:0.71), (FERROUS SULFATE\FERROUS SULFATE, DRIED:Amnesia:0.34), (FLECAINIDE:Amnesia:0.17), (HYDROMORPHONE HYDROCHLORIDE:Amnesia:0.32), (HYDROXYZINE PAMOATE:Amnesia:0.37), (HYOSCYAMINE:Amnesia:0.04), (IBRUTINIB:Amnesia:0.83), (IN SULIN GLARGINE:Amnesia:0.4), (LABETALOL\LABETALOL HYDROCHLORIDE:Amnesia:0.05), (LEDIPASVIR\SOFOSBUVIR:Amnesia:0.17), (LEVALB UTEROL HYDROCHLORIDE:Amnesia:0), (LOVASTATIN:Amnesia:1.05), (METHOCARBAMOL: Amnesia:0.05), (METHYLPREDNISOLONE SODIUM SUCCINATE:Amnesia:0.14), (METRONIDAZOLE:Amnesia:0.5), (MOXIFLOXACIN HYDROCHLORIDE:Amnesia:0.44), (NEBIVOLOL:Amnesia:0.08), (NILOTINIB:Amnesia:0.12), (PANTOPRAZOLE MAGNESIUM:Amnesia:0.05), (PEGINTERFERON BETA-1A:Amnesia:0.12), (PHENOBARBITAL:Amnesia:0.1), (PROMETHAZINE\PROMETHAZINE HYDROCHLORIDE:Amnesia:0.21), (ROPINIROLE HYDROCHLORIDE:Amnesia:0.44), (SUMATRIPTAN\SUMATRIPTAN SUCCINATE:Amnesia:0.2), (TERIFLUNOMIDE:Amnesia:0.48), (TOFACITINIB CITRATE:Amnesia:0.14), (TRIAMCINOLONE ACETONIDE:Amnesia:0.36), (TRIAMTERENE:Amnesia:0.12), (VARENICLINE TARTRATE:Amnesia:1.11), (ALBUTEROL SULFATE\IPRATROPIUM BROMIDE:Amyloidosis:0.19), (BUDESONIDE:Amyloidosis:1.02), (BUPROPION:Amyloidosis: 0.04), (CANDESARTAN CILEXETIL\HYDROCHLOROTHIAZIDE:Amyloidosis:0.06), (CARVEDILOL PHOSPHATE:Amyloidosis:0.03), (CERTOLIZUMAB PEGOL:Amyloidosis:0.92), (CLINDAMYCIN\CLINDAMYCIN PHOSPHATE:Amyloidosis:0.61), (CLOTRIMAZOLE:Amyloidosis:0.28), (DASATINIB:Amyloidosis: 0.17), (DICLOFENAC SODIUM:Amyloidosis:0.01), (DOXAZOSIN\DOXAZOSIN MESYLATE:Amyloidosis:0.16), (EPINEPHRINE:Amyloidosis:0.45), (EVEROLIMUS:Amyloidosis: 0.1), (EVOLOCUMAB:Amyloidosis:0.49), (FLUTICASONE:Amyloidosis:0.16), (HYDROC HLOROTHIAZIDE\LISINOPRIL:Amyloidosis:0.28), (ILOPROST:Amyloidosis:0.56), (INSULI N DETEMIR:Amyloidosis:0.08), (IPRATROPIUM:Amyloidosis:0.04), (LEDIPASVIR\SOFOSBU VIR:Amyloidosis:0.18), (LERCANIDIPINE:Amyloidosis:0.37), (LIDOCAINE:Amyloidosis:0.39), (LIDOCAINE\PRILOCAINE:Amyloidosis:0.05), (LINAGLIPTIN:Amyloidosis:0.06), (MELO XICAM:Amyloidosis:0.68), (METHYLPREDNISOLONE SODIUM SUCCINATE:Amyloidosis:0.8), (NEBIVOLOL:Amyloidosis:0.63), (NILOTINIB:Amyloidosis:0.1), (OLANZAPINE:Amyloidosis:0.07), (OMEGA-3-ACID ETHYL ESTERS:Amyloidosis:0.08), (PANTOPRAZOLE MAGNESIUM:Amyloidosis:0.07), (PRAMIPEXOLE DIHYDROCHLORIDE:Amyloidosis:0.08), (PRIMIDONE:Amyloidosis:0.02), (RABEPRAZOL E SODIUM:Amyloidosis:0.11), (RISEDRONATE SODIUM:Amyloidosis:0.29), (SACUBITRIL\VALSARTAN:Amyloidosis:0.28), (SELEXIPAG: Amyloidosis:0.32), (SOTALOL:Amyloidosis:0.01), (TERIFLUNOMIDE:Amyloidosis:0.06), (TI MOLOL:Amyloidosis:0.24), (TOFACITINIB CITRATE:Amyloidosis:0.25), (VERAPAMIL HYDROCHLORIDE:Amyloidosis:0.97), (.ALPHA.-TOCOPHEROL\RETINOL:Anaemia:0.42), (.ALPHA.1-PROTEINASE INHIBITOR HUMAN:Anaemia:1.3), (ABACAVIR SULFATE\DOLUTEGRAVIR SODIUM\LAMIVUDINE:Anaemia:0.56), (ABOBOTULINUMTOXINA:Anaemia:0.2), (ACAM PROSATE:Anaemia:0.02), (ACETAMINOPHEN\APRONALIDE\CAFFEINE\PROPYPHENA ZONE:Anaemia:0.11), (ACETAMINOPHEN\METHOCARBAMOL:Anaemia:0.64), (ADENINE:Anaemia:0.41), (ALBIGLUTIDE:Anaemia:0.16), (ALDESLEUKIN:Anaemia:0.91), (ALECTINI B:Anaemia:0.3), (ALGLUCOSIDASE ALFA:Anaemia:0.56), (ALISKIREN HEMIFUMARATE\HYDROCHLOROTHIAZIDE:Anaemia:0.11), (ALOGLIPTIN:Anaemia:0.23), (ALUMINUM SILICATE:Anaemia:0.27), (AMEZINIUM METILSULFATE:Anaemia:1.63), (AMLODIPINE BESYLATE\AZILSARTAN:Anaemia:0.03), (AMLODIPINE BESYLATE\HYDROCHLOROTHIAZIDE\OLMESARTAN MEDOXOMIL:Anaemia:0.66), (AMLODIPINE BESYLATE\HYDROCHLOROTHIAZIDE\VALSARTAN:Anaemia:1.87), (AMMONIUM GLYCYRRHIZATE\CYSTEINE HYDROCHLORIDE\GLYCINE:Anaemia:0.44), (AMOXAPINE:Anaemia:0.5), (AMOXICILLIN SODIUM:Anaemia:0.26), (AMPICILLIN SODIUM:Anaemia:0.72), (ANHYDROUS CITRIC ACID\ASPIRIN\SODIUM BICARBONATE:Anaemia:0.27), (ANTIHISTAMINES NOS:Anaemia:0.95), (APRINDINE HYDROCHLORIDE:Anaemia:0.31), (ARIPIPRAZOLE LAUROXIL:Anaemia:0.12), (ASPARAGINASE:Anaemia:0.69), (ASPIRIN\LANSOPRAZOLE: Anaemia:0.8), (ATORVASTATIN CALCIUM\EZETIMIBE:Anaemia:0.07), (ATRACURIUM BESYLATE:Anaemia:0.3), (AZITHROMYCIN MONOHYDRATE:Anaemia:0.24), (AZITHROMYCIN OR METHOTREXATE SODIUM:Anaemia:1.13), (BACITRACIN ZINC:Anaemia:0.03), (BAMIFYLLINE HYDROCHLORIDE:Anaemia:0.26), (BAZEDOXIFENE ACETATE\ESTROGENS, CONJUGATED:Anaemia:0.09), (BENIDIPINE:Anaemia:0.72), (BETAMETHASONE DIPROPIONATE\CALCIPOTRIENE HYDRATE:Anaemia:0.44), (BETAMETHASONE DIPROPIONATE\CALCIPOTRIENE\CALCIPOTRIENE HYDRATE:Anaemia:1.45), (BETAMETHASONE SODIUM PHOSPHATE\NEOMYCIN SULFATE:Anaemia:0.15), (BETAMIPRON\PANIPENEM:Anaemia:0.61), (BETHANECHOL CHLORIDE:Anaemia:1.16), (BIAPENEM:Anaemia:0.16), (BIFIDOBACTERIUM ANIMALIS LACTIS:Anaemia:0.16), (BISACODYL\GLYCERIN\MINERAL OIL\SODIUM PHOSPHATE, DIBASIC\SODIUM PHOSPHATE, MONOBASIC:Anaemia:0.86), (BISMUTH SUBSALICYLATE\CALCIUM CARBONATE:Anaemia:0.18), (BIXALOMER:Anaemia:0.39), (BLESELUMAB:Anaemia:0.98), (BOTULINUM TOXIN TYPE A:Anaemia:0.55), (BRINZOLAMIDE\TIMOLOL MALEATE:Anaemia:0.03), (BROMHEXINE:Anaemia:0.73), (BUFORMIN:Anaemia:0.11), (CA BOZANTINIB:Anaemia:0.92), (CABOZANTINIB S-MALATE:Anaemia:0.41), (CALCIUM BICARBONATE:Anaemia:0.03), (CALCIUM CARBONATE\ERGOCALCIFEROL:Anaemia:0.55), (CALCIUM\MAGNESIUM\ZINC:Anaemia: 1.4), (CALCIUM\VITAMINS:Anaemia:0.06), (CAMPHOR\DIPHENHYDRAMINE\ZINC OXIDE:Anaemia:0.26), (CAMPHOR\MENTHOL\METHYL SALICYLATE:Anaemia:0.36), (CANAGLIFLOZIN\METFORMIN HYDROCHLORIDE:Anaemia:2.04), (CARMUSTINE:Anaemia:0.45), (CARTEOLOL HYDROCHLORIDE:Anaemia:0.43), (CARVEDILOL PHOSPHATE:Anaemia:3.06), (CEFADROXIL:Anaemia:0.63), (CEFOZOPRAN HYDROCHLORIDE:Anaemia:0.26), (CEFPROZIL:Anaemia:0.52), (CEPHARANTHINE:Anaemia: 0.39), (CERIVASTATIN SODIUM:Anaemia:0.06), (CHIMAPHILA UMBELLATA\MANGANESE CHLORIDE\POPULUS TREMULOIDES\SODIUM TAUROCHOLATE:Anaemia:0.28), (CHLOROQUINE PHOSPHATE:Anaemia:0.24), (CHOLESTEROL:Anaemia:0.24), (CHOLINE BITARTRATE\CYSTEINE\INOSITOL\MAMMAL LIVER:Anaemia:0.06), (CICLOPIROX OLAMINE:Anaemia:0.71), (CIFENLINE SUCCINATE:Anaemia:0.09), (CLADRIBINE:Anaemia:0.43), (CLENBUTEROL HYDROCHLORIDE:Anaemia:0.81), (CLOPERASTINE:Anaemia:0.15), (CLOXACILLIN SODIUM:Anaemia:0.51), (COAGULATION FACTOR VIIA RECOMBINANT HUMAN:Anaemia:0.12), (COLESTILAN CHLORIDE:Anaemia:0.02), (COLISTIMETHATE SODIUM:Anaemia:0.72), (COLISTIN:Anaemia:0.91), (CYPROTERONE ACETATE:Anaemia:0.35), (CYPROTERONE ACETATE\ETHINYL ESTRADIOL:Anaemia:0.39), (DACLIZUMAB:Anaemia:2.3), (DANTROLENE SODIUM:Anaemia:1.02), (DAPAGLIFLOZIN PROPANEDIOL\METFORMIN HYDROCHLORIDE:Anaemia:0.61), (DAUNORUBICIN HYDROCHLORIDE:Anaemia:0.33), (DEQUALINIUM CHLORIDE:Anaemia:0.25), (DESLANOSIDE:Anaemia:0.39), (DICLOXACILLIN:Anaemia:0.89), (DILAZEP:Anaemia:0.96), (DIPHENIDOL HYDROCHLORIDE:Anaemia:1.04), (DISTIGMINE BROMIDE:Anaemia:0.67), (DOCUSATE SODIUM\SENNOSIDES A AND B:Anaemia:0.45), (DULAGLUTIDE:Anaemia:0.55), (ELBASVIR:Anaemia:0.05), (ELBASVIR\GRAZOPREVIR:Anaemia:0.3), (EMPAGLIFLOZIN\LINAGLIPTIN:Anaemia:1.54), (ENALAP RIL MALEATE\LERCANIDIPINE HYDROCHLORIDE:Anaemia:0.94), (ENTACAPONE:Anaemia:0.08), (EPHEDRA SINICA STEM\HERBALS:Anaemia:0.15), (EPHEDRINE:Anaemia:0.46), (ERIBULIN:Anaemia:1.18), (E RYTHROMYCIN\SULFISOXAZOLE:Anaemia:0.23), (ESTRADIOL HEMIHYDRATE:Anaemia:0.22), (ESTRADIOL\LEVONORGESTREL:Anaemia:0.02), (ESTR OGENS:Anaemia:0.88), (ESTROGENS, CONJUGATED\MEDROXYPROGESTERONE ACETATE:Anaemia:1.73), (ETHACRYNIC ACID:Anaemia:1.01), (ETHINYL ESTRADIOL\GESTODENE:Anaemia:0.82), (ETHINYL ESTRADIOL\NORETHINDRONE:Anaemia:1.06), (ETHIONAMIDE:Anaemia:0.17), (ETIMICI N SULFATE:Anaemia:0.99), (ETRETINATE:Anaemia:1.71), (FAROPENEM SODIUM:Anaemia:0.77), (FELBAMATE:Anaemia:0.42), (FESOTERODINE:Anaemia:0.04), (F EXOFENADINE\PSEUDOEPHEDRINE:Anaemia:1.06), (FLAVIN ADENINE DINUCLEOTIDE:Anaemia:0.63), (FLOMOXEF:Anaemia:0.24), (FLUNARIZINE HYDROCHLORIDE:Anaemia:0.09), (FLUOROURACIL\LEUCOVORIN CALCIUM\OXALIPLATIN:Anaemia:0.44), (FORMOTEROL FUMARATE\MOMETASONE FUROATE:Anaemia:1.08), (FOSFLUCONAZOLE:Anaemia:0.42), (FOSINOPRIL SODIUM\HYDROCHLOROTHIAZIDE:Anaemia:0.05), (FROVATRIPTAN SUCCINATE:Anaemia:0.08), (FURSULTIAMINE:Anaemia:0.81), (FURSULTIAMINE HYDROCHLORIDE:Anaemia:0.07), (GABEXATE MESYLATE:Anaemia:0.51), (GADOLINIUM:Anaemia:0.8), (GARENOXACIN:Anaemia:0.05), (GLIMEPIRIDE\METFORMIN:Anaemia:0.02), (GLIMEPIRIDE\ROSIGLITAZONE MALEATE:Anaemia:0.09), (GLYCOPYRROLATE\INDACATEROL MALEATE:Anaemia:0.95), (GLYCYRRHIZIN:Anaemia:0.07), (GRAZOPREVIR:Anaemia:0.03), (GUANFACINE:Anaemia:0.85), (GUANFACINE HYDROCHLORIDE:Anaemia:0.64), (HIGH DENSITY POLYETHYLENE:Anaemia:0.02), (HORSE CHESTNUT:Anaemia:0.01), (HYDROCHLOROTHIAZIDE\METOPROLOL SUCCINATE:Anaemia:0.98), (HYDROCHLOROTHIAZIDE\RAMIPRIL:Anaemia:0.85), (HYD ROCORTISONE HEMISUCCINATE:Anaemia:0.23), (HYDROXOCOBALAMIN\PYRIDOXINE HYDROCHLORIDE\THIAMINE HYDROCHLORIDE:Anaemia:0.11), (HYOSCYAMINE SULFATE\METHENAMINE\METHYLENE BLUE\PHENYL SALICYLATE\SODIUM PHOSPHATE, MONOBASIC, MONOHYDRATE:Anaemia:0.15), (IBRITUMOMAB TIUXETAN:Anaemia:1.52), (ICOSAPENT ETHYL:Anaemia:1.3), (IFENPRODIL:Anaemia:0.17), (IMMUNE GLOBULIN NOS:Anaemia:1.59), (INSULIN ASPART\INSULIN DEGLUDEC:Anaemia:0.3), (INSULIN PORK\INSULIN PURIFIED PORK:Anaemia:0.53), (INTERFERON ALFA-2A:Anaemia:1.94), (INTERFERON GAMMA-1B:Anaemia:0.96), (ISOFLURANE:Anaemia:0.75), (IXABEPILONE:Anaemia:0.42), (LANREO TIDE:Anaemia:0.67), (LENVATINIB:Anaemia:0.71), (LEVOMEFOLATE CALCIUM:Anaemia:0.34), (LEVOSIMENDAN:Anaemia:0.78), (LEVOTHYROXINE SODIUM\POTASSIUM IODIDE:Anaemia:1.73), (LOMUSTINE:Anaemia:0.87), (LOPINAVIR:Anaemia:0.49), (LULICO NAZOLE:Anaemia:0.12), (LUTETIUM LU-177:Anaemia:0.39), (MAGNESIUM CARBONATE:Anaemia:0.08), (MAGNESIUM PIDOLATE:Anaemia:0.54), (MAGNESIUM\PYRIDOXINE:Anaemia:0.67), (MAPROTILINE HYDROCHLORIDE:Anaemia:0.06), (MECLIZINE MONOHYDROCHLORIDE\NIACIN:Anaemia:0.02), (MELPERONE:Anaemia:1.05), (MEPOLI ZUMAB:Anaemia:0.86), (METFORMIN HYDROCHLORIDE\ROSIGLITAZONE MALEATE:Anaemia:0.43), (METFORMIN HYDROCHLORIDE\SAXAGLIPTIN:Anaemia:0.61), (METFORMIN HYDROCHLORIDE\SAXAGLIPTIN HYDROCHLORIDE:Anaemia:0.87), (METFORMIN PAMOATE:Anaemia:1.08), (METFORMIN\SITAGLIPTIN:Anaemia:0.56), (METHENAMINE MANDELATE:Anaemia:0.18), (METHENOLONE ACETATE:Anaemia:0.29), (METYRAPONE:Anaemia:0.18), (MEXILETINE:Anaemia:0.68), (M IGLUSTAT:Anaemia:0.79), (MILRINONE LACTATE:Anaemia:0.31), (MITIGLINIDE CALCIUM HYDRATE\VOGLIBOSE:Anaemia:0.43), (NABIXIMOLS:Anaemia:0), (NALBUPHINE HYDROCHLORIDE:Anaemia:1.15), (NALOXONE HYDROCHLORIDE:Anaemia:0.18), (NAPROXEN SODIUM\SUMATRIPTAN SUCCINATE:Anaemia:0.03), (NICERGOLINE:Anaemia:0.05), (NISOLDIPINE:Anaemia:0.31), (OPIPRAMOL:Anaemia:1.12), (ORPHENADRINE:Anaemia:1.09), (OXYBUTYNIN\OXYBUT YNIN CHLORIDE:Anaemia:0.58), (PAZUFLOXACIN MESILATE:Anaemia:0.3), (PEGINTERFERON ALFA-2A OR PEGINTERFERON ALFA-2B:Anaemia:0.78), (PEGINTERFERON BETA-1A:Anaemia:1.68), (PENTAERYTHRITOL TETRANITRATE:Anaemia:0.04), (PERPHENAZINE:Anaemia:0.11), (PHENAZOPYRIDINE HYDROCHLORIDE:Anaemia:0.62), (PHENTOLAMINE:Anaemia:0.12), (PHLOROGLUCINO L\1,3,5-TRIMETHOXYBENZENE:Anaemia:1.43), (PHOSPHORUS:Anaemia:0.42), (PIMECROLIMUS:Anaemia:0.77), (PIMOBENDAN:Anaemia:2.19), (PIRACETAM:Anaemia:0.01), (PIZOTYLINE:Anaemia:0.32), (PLASMA PROTEIN FRACTION (HUMAN):Anaemia:0.15), (POLYMYXIN:Anaemia:0.08), (POLYMYXIN B SULFATE\TRIMETHOPRIM SULFATE:Anaemia:0.36), (PORFIMER SODIUM:Anaemia:0.75), (POTASSIUM CHLORIDE\SODIUM CHLORIDE\SODIUM LACTATE:Anaemia:0.35), (POTASSIUM IODIDE:Anaemia:0.97), (POTASSIUM PHOSPHATE\SODIUM PHOSPHATE:Anaemia:0.11), (PROCARBAZINE HYDROCHLORIDE:Anaemia:0.08), (PROCATEROL HYDROCHLORIDE:Anaemia:0.84), (PROTRIPTYLINE HYDROCHLORIDE:Anaemia:0.35), (PYGEUM:Anaemia:0.16), (PYRIMETHAMINE:Anaemia: 0.8), (QUINACRINE:Anaemia:1.25), (RADIATION THERAPY:Anaemia:1.85), (REMIFENTANIL:Anaemia:0.66), (RHUBARB:Anaemia:0.28), (RIL PIVIRINE HYDROCHLORIDE:Anaemia:0.87), (RISEDRONIC ACID:Anaemia:0.24), (ROPIVACAINE:Anaemia:0.2), (ROXATIDINE ACETATE HYDROCHLORIDE:Anaemia:0.17), (RUFINAMIDE:Anaemia:0.17), (RUPATADINE FUMARATE:Anaemia:0.28), (SAPROPTERIN DIHYDROCHLORIDE:Anaemia:0.28), (SARPOGRELATE HYDROCHLORIDE:Anaemia:0.3), (SAXAGLIPTIN:Anaemia:0.1), (SCOPOLAMINE HYDROBROMIDE:Anaemia:0.99), (SELEGILINE:Anaemia:0.46), (SENNOSIDE B:Anaemia:0.3), (SERRAPEPTASE:Anaemia:0.25), (SITAFLOXACIN:Anaemia:0.27), (SIVELE STAT SODIUM:Anaemia:0.3), (SODIUM GUALENATE MONOHYDRATE:Anaemia:0.29), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Anaemia:0.13), (STREPTOMYCIN\STREPTOMYCIN SULFATE:Anaemia:0.88), (SUPLATAST TOSILATE:Anaemia:0.9), (TAFAMIDIS MEGLUMINE:Anaemia:0.69), (TELOTRISTAT ETHYL:Anaemia:0.73), (THIAMYLAL SODIUM:Anaemia:0.42), (THIOCTIC ACID:Anaemia:1.11), (THIOPENTAL:Anaemia:0.79), (THROMBOPOIETIN:Anaemia:0.46), (T IMOLOL\TRAVOPROST:Anaemia:0.14), (TOFACITINIB:Anaemia:0.05), (TREPROSTINIL DIOLAMINE:Anaemia:0.65), (TRIHEXYPHENIDYL HYDROCHLORIDE:Anaemia:0.05), (TRIMIPRAMINE MALEATE:Anaemia:1.36), (TRIPTORELIN:Anaemia:0.03), (TRIPTORELIN PAMOATE:Anaemia:0.9), (VALDECOXIB:Anaemia:0.59), (VANDETANIB:Anaemia:0.15), (VI NBLASTINE:Anaemia:0.28), (VINDESINE SULFATE:Anaemia:0.12), (VONOPRAZAN:Anaemia:0.4), (YEAST:Anaemia:0.74), (ZICONOT IDE ACETATE:Anaemia:0.6), (AMIODARONE:Anaemia prophylaxis:0.89), (ANASTROZOLE:Anaemia prophylaxis:0.62), (BUPROPION HYDROCHLORIDE:Anaemia prophylaxis:0.08), (CANDESARTAN:Anaemia prophylaxis:0.75), (CANDESARTAN CILEXETIL:Anaemia prophylaxis:0.7), (CANDESARTAN CILEXETIL\HYDROCHLOROTHIAZIDE:Anaemia prophylaxis:0.37), (CEFOTAXIME SODIUM:Anaemia prophylaxis:0.05), (CHOLESTYRAMINE:Anaemia prophylaxis:0.12), (CLINDAMYCIN\CLINDAMYCIN PHOSPHATE:Anaemia prophylaxis:0.65), (CORTISONE\HYDROCORTISONE:Anaemia prophylaxis:0.15), (DANAZOL:Anaemia prophylaxis:0.08), (DASATINIB:Anaemia prophylaxis:0.71), (DEXAMETHASONE ACETATE:Anaemia prophylaxis:0.01), (EPINEPHRINE:Anaemia prophylaxis:0.08), (EPIRUBICIN:Anaemia prophylaxis:0.03), (EZETIMIBE:Anaemia prophylaxis:0.14), (GADOLINIUM:Anaemia prophylaxis:0.61), (HYDROCHLOROTHIAZIDE\LISINOPRIL:Anaemia prophylaxis:0.01), (HYDROMORPHONE:Anaemia prophylaxis:0.02), (HYDROXYCHLOROQUINE SULFATE:Anaemia prophylaxis:0.37), (ILOPROST:Anaemia prophylaxis:0.23), (INDAPAMIDE\PERINDOPRIL:Anaemia prophylaxis:0.36), (IRINOTECAN:Anaemia prophylaxis:0.41), (ISOSORBIDE MONONITRATE:Anaemia prophylaxis:0.2), (LEDIPASVIR\SOFOSBUVIR:Anaemia prophylaxis:0.65), (LOVASTATIN:Anaemia prophylaxis:0.56), (MELOXICAM:Anaemia prophylaxis:0.36), (METRONIDAZOLE:Anaemia prophylaxis:1.04), (MICAFUNGIN:Anaemia prophylaxis:0.03), (NAPROXEN:Anaemia prophylaxis:0.71), (NITROFURANTOIN:Anaemia prophylaxis:0), (NYSTATIN:Anaemia prophylaxis:0.25), (SACUBITRIL\VALSARTAN:Anaemia prophylaxis:0.47), (SECUKINUMAB:Anaemia prophylaxis:0.15), (SELEXIPAG:Anaemia prophylaxis:0.3), (SOTALOL:Anaemia prophylaxis:0.32), (THALIDOMIDE:Anaemia prophylaxis:0.74), (TOCILIZUMAB:Anaemia prophylaxis:0.37), (ACENOCOUMAROL:Anaesthesia:0.02), (ACETAMINOPHEN\BUTALBITAL\CAFFEINE:Anaesthesia:0.59), (ACETAMINOPHEN\OXYCODONE\OXYCODONE HYDROCHLORIDE:Anaesthesia:0.29), (ADO-TRASTUZUMAB EMTANSINE:Anaesthesia:0.24), (ALCOHOL:Anaesthesia:0.9), (ALEMTUZUMAB:Anaesthesia: 0.29), (AMIKACIN SULFATE:Anaesthesia:0.22), (AMLODIPINE\VALSARTAN:Anaesthesia:0.14), (ATOVAQUO NE:Anaesthesia:0.2), (BENAZEPRIL HYDROCHLORIDE:Anaesthesia:0.29), (BENAZEPRIL\BENAZEPRIL HYDROCHLORIDE:Anaesthesia:0.31), (BUDESONIDE\FORMOTEROL FUMARATE:Anaesthesia:0), (CALCITONIN SALMON:Anaesthesia:0.39), (CANAKINUMAB:Anaesthesia:0.18), (CANDESARTAN CILEXETIL\HYDROCHLOROTHIAZIDE:Anaesthesia:0.94), (CARBON DIOXIDE:Anaesthesia:0.47), (CARVEDILOL PHOSPHATE:Anaesthesia:0.3), (CEFOTAXIME SODIUM:Anaesthesia:1.15), (CEFOTAXIME\CEFOTAXIME SODIUM:Anaesthesia:0.2), (CHLORTHALIDONE:Anaesthesia:0.22), (DABIGATRAN:Anaesthesia: 0.29), (DACLIZUMAB:Anaesthesia:0.17), (DALFAMPRIDINE:Anaesthesia:0.29), (DARBE POETIN ALFA:Anaesthesia:0.07), (DASATINIB:Anaesthesia:0.79), (DIMENHYDRINATE:Anaesthesia: 0.56), (DOBUTAMINE HYDROCHLORIDE:Anaesthesia:0.85), (DRONABINOL:Anaesthesia:0.65), (EDOXABAN TOSYLATE:Anaesthesia:0.18), (ELETRIPTAN HYDROBROMIDE:Anaesthesia:0.48), (EPIRUBICIN:Anaesthesia:0.37), (ESTROGENS, CONJUGATED:Anaesthesia:0.5), (ESTROGENS, CONJUGATED\MEDROXYPROGESTERONE ACETATE:Anaesthesia:0.3), (ESZOPICLONE:Anaesthesia:0.85), (ETORICOXIB:Anaesthesia:00.21), (EVOLOCUMAB:Anaesthesia:0.64), (FESOTERODINE FUMARATE:Anaesthesia:0.17), (FIBRINOGEN HUMAN:Anaesthesia:0.36), (FLECAINIDE:Anaesthesia:1.01), (FLUTICASONE FUROATE\SALMETEROL XINAFOATE:Anaesthesia:0.55), (FLUTICASONE\FLUTICASONE PROPIONATE:Anaesthesia:0.24), (FONDAPARINUX:Anaesthesia:0.29), (GLYCOPYRRONIU M\INDACATEROL:Anaesthesia:0.29), (GOSERELIN ACETATE:Anaesthesia:0.06), (HERBALS:Anaesthesia:0.06), (HYDROCHLOROTHIAZIDE\IR BESARTAN:Anaesthesia:0.68), (HYDROCHLOROTHIAZIDE\LISINOPRIL:Anaesthesia:0.92), (HYDROCHLOROTHIAZIDE\TELMISARTAN:Anaesthesia:0.01), (HYDROXYCHLOROQU INE DIPHOSPHATE:Anaesthesia:0.09), (HYDROXYCHLOROQUINE SULFATE:Anaesthesia:0.37), (IBRUTINIB:Anaesthesia:1.21), (IMATINIB MESYLATE:Anaesthesia:0.46), (IMIPENEM:Anaesthesia:0.47), (IMIPRAMINE HYDROCHLORIDE:Anaesthesia:0.36), (IPRATROPIUM:Anaesthesia:0.79), (ITRACONAZOL E:Anaesthesia:0.4), (LINAGLIPTIN:Anaesthesia:0.3), (MEGESTROL ACETATE:Anaesthesia:0.08), (METARAMINOL:Anaesthesia:0.57), (METHENAMINE HIPPURATE:Anaesthesia:0.44), (MICAFUNGIN:Anaesthesia:0.16), (MORPHINE\MORPHINE HYDROCHLORIDE\MORPHINE SULFATE\MORPHINE TARTRATE:Anaesthesia:0.21), (NABUMETONE:Anaesthesia:0.08), (NALOXONE HYDROCHLORIDE\OXYCODONE HYDROCHLORIDE:Anaesthesia:0.69), (NEBIVOLOL:Anaesthesia:0.85), (NORTRIPTYLINE: Anaesthesia:0.45), (OCTREOTIDE ACETATE:Anaesthesia:0.14), (OMEGA-3-ACID ETHYL ESTERS:Anaesthesia:0.44), (OXCARBAZEPINE:Anaesthesia:2.37), (OXYBUTYNIN CHLORIDE:Anaesthesia:0.24), (PANTOPRAZOLE MAGNESIUM:Anaesthesia:0.4), (PAZOPANIB HYDROCHLORIDE:Anaesthesia:0.09), (PEMETREXED DISODIUM:Anaesthesia:0.03), (PENICILLIN:Anaesthesia:0.89), (PHENYLEPHRINE HYDROCHLORIDE\TROPICAMIDE:Anaesthesia:0.07), (POLYMYXIN B SULFATE\TRIMETHOPRIM SULFATE:Anaesthesia:0.29), (PONATINIB:Anaesthesia:0.39), (POSACONAZOLE:Anaesthesia: 0.42), (PRAMIPEXOLE DIHYDROCHLORIDE:Anaesthesia:0.32), (PREDNISOLONE SODIUM PHOSPHATE:Anaesthesia:0.22), (PROBIOTICS NOS:Anaesthesia:0.85), (RANOLAZINE:Anaesthesia:0.85), (RIZATRIPTAN BENZOATE:Anaesthesia:0.33), (ROFLUMILAST:Anaesthesia:0.11), (ROPINIROLE HYDROCHLORIDE:Anaesthesia:0.22), (SACUBITRIL\VALSARTAN:Anaesthesia:0.6), (SCOP OLAMINE HYDROBROMIDE:Anaesthesia:0.02), (SUMATRIPTAN:Anaesthesia:0.41), (TAZOBACTAM: Anaesthesia:0.11), (TERIFLUNOMIDE:Anaesthesia:0.34), (TETRACAINE\TETRACAINE HYDROCHLORIDE:Anaesthesia:0.1), (TETRACYCLINE:Anaesthesia:0.55), (TINZAPARIN SODIUM:Anaesthesia:0.29), (TIZANIDINE HYDROCHLORIDE:Anaesthesia:0.89), (TOCILIZUMAB:Anaesthesia:0.39), (UMECLIDINIUM BROMIDE\VILANTEROL TRIFENATATE:Anaesthesia:0.62), (VALGANCICLOVIR HYDROCHLORIDE:Anaesthesia:1.09), (VARENICLINE TARTRATE:Anaesthesia:0.93), (VINCRISTINE SULFATE:Anaesthesia:0.72), (VITAMIN A:Anaesthesia:0.01), (BUPIVACAINE\EPINEPHRINE:Anaesthesia procedure:0.15), (KETAMINE HYDROCHLORIDE:Anaesthesia procedure:0.04), (NEOSTIGMINE:Anaesthetic premedication:0.89), (THIOPENTAL:Anaesthetic premedication:0), (EVOLOCUMAB:Analgesic intervention supportive therapy:0.08), (.ALPHA.1-PROTEINASE INHIBITOR HUMAN:Analgesic therapy:0.93), (ABOBOTULINUMTOXINA:Analgesic therapy:0.35), (ACEBUTOLOL HYDROCHLORIDE:Analgesic therapy:0.65), (ACETAMINOPHEN\PROPOXYPHENE NAPSYLATE:Analgesic therapy:0.59), (ADENINE:Analgesic therapy:0.7), (ADENOSINE:Analgesic therapy:0.01), (ADENOSINE TRIPHOSPHATE:Analgesic therapy:0.26), (AGALSIDASE BETA:Analgesic therapy:1.09), (ALBUTEROL\IPRATROPIUM:Analgesic therapy:0.7), (ALDESLEUKIN:Analgesic therapy:0.88), (ALECTINIB:Analgesic therapy:0.14), (ALISKIREN HEMIFUMARATE:Analgesic therapy:0.91), (ALUMINUM HYDROXIDE\MAGNESIUM OXIDE\OXETHAZAINE:Analgesic therapy:0.3), (AMBROXOL\CLENBUTEROL:Analgesic therapy:0.85), (AMEZINIUM METILSULFATE:Analgesic therapy:0.84), (AMILORIDE HYDROCHLORIDE\HYDROCHLOROTHIAZIDE:Analgesic therapy:1.29), (AMINOSALICYLIC ACID:Analgesic therapy:0.05), (AMISULPRIDE:Analgesic therapy:1.67), (AMLODIPINE BESYLATE\OLMESARTAN MEDOXOMIL:Analgesic therapy:0.56), (AMLODIPINE\PERINDOPRIL:Analgesic therapy:0.84), (AMMONIUM CHLORIDE\DEXTROMETHORPHAN\SODIUM CITRATE:Analgesic therapy:0.16), (AMMONIUM GLYCYRRHIZATE:Analgesic therapy:0.14), (AMMONIUM GLYCYRRHIZATE\CYSTEINE HYDROCHLORIDE\GLYCINE:Analgesic therapy:0.21), (AMOXICILLIN\CLARITHROMYCIN\ESOMEPRAZOLE:Analgesic therapy:0.85), (AMPICILLIN SODIUM:Analgesic therapy:0.11), (ANAGLIPTIN:Analgesic therapy:0.15), (ANAGRELIDE:Analgesic therapy:0.21), (ASPIRIN\CLOPIDOGREL:Analgesic therapy:0.46), (ASPIRIN\DIPYRIDAMOLE:Analgesic therapy:0.4), (ASPIRIN\MAGNESIUM OXIDE:Analgesic therapy:0.74), (ATROPINE\DIPHENOXYLATE:Analgesic therapy:0.18), (AZELASTINE HYDROCHLORIDE\FLUTICASONE PROPIONATE:Analgesic therapy:0.08), (BELATACEPT:Analgesic therapy:0.4), (BENZTROPINE:Analgesic therapy:0.45), (BENZTROPINE MESYLATE:Analgesic therapy:0.29), (BEPRIDIL:Analgesic therapy:1.19), (BETAMETHASONE BUTYRATE PROPIONATE:Analgesic therapy:0.64), (BETAMETHASONE DIPROPIONATE\CALCIPOTRIENE\CALCIPOTRIENE HYDRATE:Analgesic therapy:0.32), (BETAMETHASONE SODIUM PHOSPHATE\NEOMYCIN SULFATE:Analgesic therapy:0.02), (BETAMETHASONE\CALCIPOTRIENE:Analgesic therapy:0.7), (BETAMIPRON\PANIPENEM:Analgesic therapy:0.24), (BICARBONATE ION:Analgesic therapy:0.63), (BIFIDOBACTERIUM LONGUM SUBSP. INFANTIS:Analgesic therapy:0.58), (BIPERIDEN HYDROCHLORIDE:Analgesic therapy:0.68), (BLESELUMAB:Analgesic therapy:0.15), (BLINATUMOMAB:Analgesic therapy:1.4), (BOCOCIZUMAB:Analgesic therapy:0.29), (BOTULINUM TOXIN TYPE A:Analgesic therapy:0.15), (BOTULISM ANTITOXIN:Analgesic therapy:0.13), (BRIMONIDINE TARTRATE\TIMOLOL MALEATE:Analgesic therapy:0.07), (BUDESONIDE\FORMOTEROL FUMARATE:Analgesic therapy:1.31), (BUPIVACAINE\EPINEPHRINE:Analgesic therapy:0.46), (CALCIPOTRIENE:Analgesic therapy:0.64), (CALCIUM ACETATE\MAGNESIUM ACETATE\POTASSIUM ACETATE\SODIUM ACETATE\SODIUM CHLORIDE:Analgesic therapy:0.13), (CALCIUM CARBONATE\ERGOCALCIFEROL\RETINOL:Analgesic therapy:0.68), (CALCIUM CITRATE\VITAMIN D:Analgesic therapy:0.09), (CALCIUM\MAGNESIUM:Analgesic therapy:0.54), (CALCIUM\MAGNESIUM\ZINC:Analgesic therapy:0.59), (CAMPHOR\MENTHOL\METHYL SALICYLATE:Analgesic therapy:0.11), (CANAGLIFLOZIN\METFORMIN HYDROCHLORIDE:Analgesic therapy:1.42), (CARBON DIOXIDE:Analgesic therapy:0.04), (CARVEDILOL PHOSPHATE:Analgesic therapy:1.77), (CEFOTAXIME\CEFOTAXIME SODIUM:Analgesic therapy:0.95), (CEFPODOXIME PROXETIL:Analgesic therapy:0.89), (CELLULASE\DIASTASE\LIPASE:Analgesic therapy:0.22), (CEPHARANTHINE:Analgesic therapy:0.29), (CEVIMELINE HYDROCHLORIDE:Analgesic therapy:0.14), (CHIMAPHILA UMBELLATA\MANGANESE CHLORIDE\POPULUS TREMULOIDESSODIUM TAUROCHOLATE:Analgesic therapy:0.2), (CHLOROTHIAZIDE:Analgesic therapy:0.2), (CIFENLINE:Analgesic therapy:0.53), (CIPROFIBRATE:Analgesic therapy:0.88), (CLADRIBINE:Analgesic therapy:0.23), (CLENBUTEROL HYDROCHLORIDE:Analgesic therapy:0.63), (CLOMIPRAMINE HYDROCHLORIDE:Analgesic therapy:0.83), (COD LIVER OIL:Analgesic therapy:0.71), (COLESTIPOL:Analgesic therapy:0.04), (CORTICOTROPIN:Analgesic therapy:0.72), (CROTALIDAE POLYVALENT IMMUNE FAB (OVINE):Analgesic therapy:0.22), (CROTAMITON:Analgesic therapy:0.09), (CYPROTERONE ACETATE:Analgesic therapy:0.05), (DANAPAROID SODIUM:Analgesic therapy:0.02), (DAPAGLIFLOZIN PROPANEDIOL\METFORMIN HYDROCHLORIDE:Analgesic therapy:0.08), (DASABUVIR:Analgesic therapy:0.59), (DESVENLAFAXINE:Analgesic therapy:0.39), (DEXTROAMPHETAMINE SULFATE:Analgesic therapy:0.51), (DILAZEP:Analgesic therapy:0.74), (DIMETHICONE\SILICON DIOXIDE\SIMETHICONE:Analgesic therapy:0.22), (DIPHENHYDRAMINE HYDROCHLORIDE\LIDOCAINE HYDROCHLORIDE\NYSTATIN:Analgesic therapy:0.1), (DOFETILIDE:Analgesic therapy:1.18), (DOLASETRON MESYLATE:Analgesic therapy:0.68), (DOLUTEGRAVIR:Analgesic therapy:0.22), (DOXEPIN HYDROCHLORIDE:Analgesic therapy:0.98), (DROXIDOPA:Analgesic therapy:0.08), (DUTASTERIDE\TAMSULOSIN HYDROCHLORIDE:Analgesic therapy:0.79), (ECABET SODIUM:Analgesic therapy:0.07), (ECONAZOLE:Analgesic therapy:1.79), (EDARAVONE:Analgesic therapy:0.64), (ELBASVIR\GRAZOPREVIR:Analgesic therapy:0.34), (EMPAGLIFLOZIN\LINAGLIPTIN:Analgesic therapy:1.02), (EMTRICITABINE:Analgesic therapy:0.13), (ENALAPRIL MALEATEHYDROCHLOROTHIAZIDE:Analgesic therapy:0.34), (ERIBULIN:Analgesic therapy:1), (ERYTHROMYCIN LACTOBIONATE:Analgesic therapy:0.12), (ESTRONE\SUS SCROFA OVARY:Analgesic therapy:0.78), (ETHAMBUTOL HYDROCHLORIDE:Analgesic therapy:0.95), (ETHINYL ESTRADIOL\NORGESTREL:Analgesic therapy:0.07), (ETHYL ICOSAPENTATE\ICOSAPENT:Analgesic therapy:0.37), (ETRETINATE:Analgesic therapy:1.38), (FAROPENEM SODIUM:Analgesic therapy:0.32), (FENOTEROL HYDROBROMIDE:Analgesic therapy:0.84), (FERRIC CARBOXYMALTOSE:Analgesic therapy:0.15), (FERROUS GLYCINE SULFATE:Analgesic therapy:0.16), (FERROUS SULFATE ANHYDROUS:Analgesic therapy:0.46), (FERROUS SULFATE\GLYCINE:Analgesic therapy:1.28), (FEXOFENADINE\PSEUDOEPHEDRINE:Analgesic therapy:0.5), (FISH OIL\OMEGA-3 FATTY ACIDS:Analgesic therapy:0.2), (FLAVIN ADENINE DINUCLEOTIDE:Analgesic therapy:0.4), (FLUPHENAZINE:Analgesic therapy:0.01), (FLURAZEPAM\FLURAZEPAM HYDROCHLORIDE:Analgesic therapy:0.53), (FLUTICASONE PROPIONATE\FORMOTEROL FUMARATE:Analgesic therapy:0.48), (FORMOTEROL FUMARATE\MOMETASONE FUROATE:Analgesic therapy:1.01), (FOSFLUCONAZOLE:Analgesic therapy:0.25), (FURSULTIAMINE:Analgesic therapy:0.53), (GLYCOPYRROLATE\INDACATEROL MALEATE:Analgesic therapy:0.6), (GUANABENZ ACETATE:Analgesic therapy:0.11), (GUANFACINE:Analgesic therapy:0.18), (GUANFACINE HYDROCHLORIDE:Analgesic therapy:0.08), (HALOTHANE:Analgesic therapy:0.08), (HYDROCHLOROTHIAZIDE\LOSARTAN:Analgesic therapy:0.19), (HYDROCHLOROTHIAZIDE\TELMISARTAN:Analgesic therapy:1.59), (HYDROCORTISONE HEMISUCCINATE:Analgesic therapy:0.08), (HYOSCYAMINE:Analgesic therapy:0.81), (IBRITUMOMAB TIUXETAN:Analgesic therapy:0.84), (IBUDILAST:Analgesic therapy:0.04), (IGURATIMOD:Analgesic therapy:0.28), (IMMUNE GLOBULIN NOS:Analgesic therapy:0.49), (INDACATEROL:Analgesic therapy:1.07), (INTERFERON:Analgesic therapy:1.09), (INTERFERON ALFA-2A:Analgesic therapy:1.36), (INTERFERON ALFA-2B:Analgesic therapy:1.42), (ISONIAZID\RIFAMPIN:Analgesic therapy:0.31), (ITOPRIDE:Analgesic therapy:0.22), (IVABRADINE HYDROCHLORIDE:Analgesic therapy:0.12), (LABETALOL HYDROCHLORIDE:Analgesic therapy:1.68), (LACTASE:Analgesic therapy:0.58), (LACTOBACILLUS ACIDOPHILUS\LACTOBACILLUS DELBRUECKII SUBSP. BULGARICUS:Analgesic therapy:0.23), (LACTOBACILLUS RHAMNOSUS:Analgesic therapy:0.84), (LANREOTIDE:Analgesic therapy:0.2), (LAPATINIB DITOSYLATE:Analgesic therapy:0.96), (LEVOCARNITINE HYDROCHLORIDE:Analgesic therapy:0.58), (LEVODOPA:Analgesic therapy:0.27), (LINSIDOMINE:Analgesic therapy:0.24), (LISDEXAMFETAMINE DIMESYLATE:Analgesic therapy:0.99), (LOMITAPIDE MESYLATE:Analgesic therapy:0.45), (LOTEPREDNOL ETABONATE:Analgesic therapy:0.41), (LURASIDONE HYDROCHLORIDE:Analgesic therapy:0.19), (LUTETIUM LU-177:Analgesic therapy:0.02), (LYSOZYME HYDROCHLORIDE:Analgesic therapy:0.48), (MAGNESIUM ASPARTATE HYDROCHLORIDE:Analgesic therapy:0.8), (MAGNESIUM SULFATE HEPTAHYDRATE:Analgesic therapy:0.04), (MAXACALCITOL:Analgesic therapy:0.63), (MELPERONE:Analgesic therapy:0.35), (METHENAMINE HIPPURATE:Analgesic therapy:1.68), (METHENOLONE ENANTHATE:Analgesic therapy:1.81), (METHYLPREDNISOLONE OR METHYLPREDNISOLONE ACETATE:Analgesic therapy:0.01), (MEXILETINE:Analgesic therapy:0.17), (MICONAZOLE NITRATE:Analgesic therapy:0.66), (MITIGLINIDE:Analgesic therapy:0.93), (MITIGLINIDE CALCIUM HYDRATEVOGLIBOSE:Analgesic therapy:0.23), (MIZORIBINE:Analgesic therapy:1.6), (MOEXIPRIL HYDROCHLORIDE:Analgesic therapy:0.3), (NALTREXONE:Analgesic therapy:0.39), (OLOPATADINE:Analgesic therapy:0.51), (OPIPRAMOL:Analgesic therapy:0.29), (ORPHENADRINE:Analgesic therapy:0.54), (OXYBUTYNIN\OXYBUTYNIN CHLORIDE:Analgesic therapy:0.01), (PARICALCITOL:Analgesic therapy:0.42), (PAZUFLOXACIN MESILATE:Analgesic therapy:0.27), (PEGINTERFERON BETA-1A:Analgesic therapy:0.88), (PEGZEREPOETIN ALFA:Analgesic therapy:0.33), (PHENELZINE:Analgesic therapy:0.24), (PILOCARPINE:Analgesic therapy:0.52), (PILSICAINIDE:Analgesic therapy:0.28), (PIMOBENDAN:Analgesic therapy:1.3), (PIPERACILLIN SODIUM:Analgesic therapy:0.19), (PIRENZEPINE:Analgesic therapy:0.22), (PNEUMOCOCCAL VACCINE, POLYVALENT 23:Analgesic therapy:0.42), (POLYETHYLENE GLYCOL 3350\POTASSIUM CHLORIDE\SODIUM BICARBONATE\SODIUM CHLORIDE\SODIUM SULFATE ANHYDROUS:Analgesic therapy:0.36), (POLYMYXIN B SULFATE:Analgesic therapy:0.01), (POLYSTYRENE:Analgesic therapy:0.07), (PORFIMER SODIUM:Analgesic therapy:0.44), (POTASSIUM CITRATE\TRISODIUM CITRATE DIHYDRATE:Analgesic therapy:1.19), (PREDNISOLONE SODIUM PHOSPHATE:Analgesic therapy:1.22), (PRISTINAMYCIN:Analgesic therapy:0.68), (PROPAFENONE:Analgesic therapy:1.01), (QUINACRINE:Analgesic therapy:0.4), (QUINAPRIL:Analgesic therapy:0.68), (RETINOL:Analgesic therapy:1.01), (RIFABUTIN:Analgesic therapy:0.09), (RILPIVIRINE HYDROCHLORIDE:Analgesic therapy:0.3), (RIVASTIGMINE TARTRATE:Analgesic therapy:1.59), (ROFLUMILAST:Analgesic therapy:1.67), (ROXITHROMYCIN:Analgesic therapy:0.04), (SARPOGRELATE HYDROCHLORIDE:Analgesic therapy:0.31), (SELEXIPAG:Analgesic therapy:0.67), (SENNOSIDE B:Analgesic therapy:0.21), (SEVELAMER HYDROCHLORIDE:Analgesic therapy:1.19), (SIPULEUCEL-T:Analgesic therapy:0.37), (SODIUM GUALENATE MONOHYDRATE:Analgesic therapy:0.22), (SOFOSBUVIR\VELPATASVIR:Analgesic therapy:0.22), (SULBACTAM SODIUM:Analgesic therapy:0.79), (SULODEXIDE:Analgesic therapy:0.07), (SUPLATAST TOSILATE:Analgesic therapy:0.98), (TABALUMAB:Analgesic therapy:0.07), (TAFLUPROST:Analgesic therapy:0.06), (TEDUGLUTIDE\WATER:Analgesic therapy:0.43), (TEMOCAPRIL HYDROCHLORIDE:Analgesic therapy:0.62), (TEMSIROLIMUS:Analgesic therapy:0.47), (TETRACYCLINE:Analgesic therapy:1.37), (THIAMYLAL:Analgesic therapy:0.18), (THIOCTIC ACID:Analgesic therapy:0.15), (THROMBOPOIETIN:Analgesic therapy:0.25), (TICLOPIDINE:Analgesic therapy:0.52), (TOFACITINIB:Analgesic therapy:0.15), (TRAVOPROST:Analgesic therapy:0.38), (TRIHEXYPHENIDYL:Analgesic therapy:0.66), (TRIHEXYPHENIDYL HYDROCHLORIDE:Analgesic therapy:0.12), (TRIPTORELIN PAMOATE:Analgesic therapy:0.26), (UMECLIDINIUM BROMIDE:Analgesic therapy:0.84), (UMECLIDINIUM BROMIDE\VILANTEROL TRIFENATATE:Analgesic therapy:1.22), (VINDESINE SULFATE:Analgesic therapy:0.03), (VONOPRAZAN:Analgesic therapy:0.12), (ZILEUTON:Analgesic therapy:0.39), (ZIV-AFLIBERCEPT:Analgesic therapy:0.55), (ACETAMINOPHEN\PROPOXYPHENE NAPSYLATE:Anaphylactic reaction:0.12), (ACLIDINIUM BROMIDE:Anaphylactic reaction:0.83), (ALBIGLUTIDE:Anaphylactic reaction:0.16), (ALCOHOL:Anaphylactic reaction:0.12), (ALENDRONIC ACID:Anaphylactic reaction:0.35), (AMLODIPINE BESYLATE\HYDROCHLOROTHIAZIDE\OLMESARTAN MEDOXOMIL:Anaphylactic reaction:0.08), (AMLODIPINE\VALSARTAN:Anaphylactic reaction:0.19), (BALSALAZIDE DISODIUM:Anaphylactic reaction:0.27), (BENAZEPRIL\BENAZEPRIL HYDROCHLORIDE:Anaphylactic reaction:0.78), (BETAMETHASONE\CALCIPOTRIENE:Anaphylactic reaction:0.09), (BRIMONIDINE TARTRATE\TIMOLOL MALEATE:Anaphylactic reaction:0.08), (BRINZOLAMIDE:Anaphylactic reaction:0.74), (BUPRENORPHINE HYDROCHLORIDE:Anaphylactic reaction:0.26), (BUPRENORPHINE HYDROCHLORIDE\NALOXONE HYDROCHLORIDE:Anaphylactic reaction:0.63), (BUTYLSCOPOLAMINE BROMIDE:Anaphylactic reaction:0.24), (CALCITONIN:Anaphylactic reaction:0.01), (CALCITONIN SALMON:Anaphylactic reaction:0.88), (CALCIUM\MAGNESIUM\ZINC:Anaphylactic reaction:0.08), (CANAGLIFLOZIN\METFORMIN HYDROCHLORIDE:Anaphylactic reaction:0.16), (CANAKINUMAB:Anaphylactic reaction:0.79), (CANDESARTAN:Anaphylactic reaction:0.88), (CANDESARTAN CILEXETIL:Anaphylactic reaction:0.78), (CANDESARTAN CILEXETIL\HYDROCHLOROTHIAZIDE:Anaphylactic reaction:0.58), (CAPTOPRIL:Anaphylactic reaction:0.61), (CARFILZOMIB:Anaphylactic reaction:0.81), (CASANTHRANOL\DOCUSATE SODIUM:Anaphylactic reaction:0.07), (CEFOTAXIME SODIUM:Anaphylactic reaction:0.11), (CEFPROZIL:Anaphylactic reaction:0.32), (CHLOROTHIAZIDE:Anaphylactic reaction:0.2), (CORTICOTROPIN:Anaphylactic reaction:0.51), (DACLIZUMAB:Anaphylactic reaction:1.32), (DAPAGLIFLOZIN PROPANEDIOL:Anaphylactic reaction:0.23), (DASATINIB:Anaphylactic reaction:1.24), (DEFLAZACORT:Anaphylactic reaction:0.5), (DIMENHYDRINATE:Anaphylactic reaction:1), (DIPYRONE:Anaphylactic reaction:0.26), (DOXAZOSIN MESYLATE:Anaphylactic reaction:0.51), (EDOXABAN TOSYLATE:Anaphylactic reaction:0.23), (EMPAGLIFLOZIN\LINAGLIPTIN:Anaphylactic reaction:0.33), (EPHEDRINE HYDROCHLORIDE\GUAIFENESIN:Anaphylactic reaction:0.19), (EPLERENONE:Anaphylactic reaction:0.21), (ESTROGENS, CONJUGATED\MEDROXYPROGESTERONE ACETATE:Anaphylactic reaction:1.1), (ESTRONE\SUS SCROFA OVARY:Anaphylactic reaction:0.46), (EVEROLIMUS:Anaphylactic reaction:0.44), (FENOFIBRIC ACID:Anaphylactic reaction:0.19), (FINGOLIMOD HYDROCHLORIDE:Anaphylactic reaction:0.2), (FLUOCINONIDE:Anaphylactic reaction:0.8), (FLUTICASONE FUROATE\SALMETEROL XINAFOATE:Anaphylactic reaction:1.31), (FORMOTEROL:Anaphylactic reaction:0.22), (FUSIDIC ACID:Anaphylactic reaction:0.32), (GLICLAZIDE:Anaphylactic reaction:0.2), (GLYBURIDE:Anaphylactic reaction:0.49), (GLYCOPYRROLATE\INDACATEROL MALEATE:Anaphylactic reaction:0.05), (GLYCOPYRRONIUM\INDACATEROL:Anaphylactic reaction:0.7), (GUANFACINE:Anaphylactic reaction:0.4), (HERBALS\TURMERIC:Anaphylactic reaction:0.5), (HYDROCHLOROTHIAZIDE\IRBESARTAN:Anaphylactic reaction:0.55), (HYDROCODONE BITARTRATE\IBUPROFEN:Anaphylactic reaction:0.21), (HYDROCORTISONE ACETATE:Anaphylactic reaction:0.42), (HYDROXYUREA:Anaphylactic reaction:0.46), (ILOPROST:Anaphylactic reaction:0.57), (INDAPAMIDE\PERINDOPRIL:Anaphylactic reaction:0.02), (INDOMETHACIN:Anaphylactic reaction:0.8), (INFLIXIMAB-DYYB:Anaphylactic reaction:0.29), (ISOSORBIDE MONONITRATE:Anaphylactic reaction:1.24), (LABETALOL\LABETALOL HYDROCHLORIDE:Anaphylactic reaction:1.56), (LEDIPASVIR\SOFOSBUVIR:Anaphylactic reaction:0.42), (LERCANIDIPINE:Anaphylactic reaction:0.04), (LINAGLIPTIN:Anaphylactic reaction:0.57), (LOMITAPIDE MESYLATE:Anaphylactic reaction:0.36), (LUMACAFTOR:Anaphylactic reaction:0.24), (MEGESTROL ACETATE:Anaphylactic reaction:0.23), (MEPOLIZUMAB:Anaphylactic reaction:1.6), (MIRABEGRON:Anaphylactic reaction:0.42), (MOXIFLOXACIN:Anaphylactic reaction:0.65), (NALOXONE HYDROCHLORIDE\OXYCODONE HYDROCHLORIDE:Anaphylactic reaction:0.47), (NATEGLINIDE:Anaphylactic reaction:0.07), (NEBIVOLOL:Anaphylactic reaction:0.11), (NILOTINIB:Anaphylactic reaction:0.75), (NINTEDANIB:Anaphylactic reaction:0.63), (OMEPRAZOLE SODIUM:Anaphylactic reaction:0.01), (ORPHENADRINE:Anaphylactic reaction:0.04), (OSELTAMIVIR:Anaphylactic reaction:0.25), (OXAZEPAM:Anaphylactic reaction:0.07), (PAMIDRONATE DISODIUM:Anaphylactic reaction:0.26), (PANCRELIPASE:Anaphylactic reaction:0.05), (PATIROMER:Anaphylactic reaction:0.12), (PAZOPANIB HYDROCHLORIDE:Anaphylactic reaction:0.17), (PEGFILGRASTIM:Anaphylactic reaction:0.15), (PEGINTERFERON BETA-1A:Anaphylactic reaction:0.68), (PENICILLIN G:Anaphylactic reaction:0.06), (PHENOBARBITAL:Anaphylactic reaction:1.22), (PHENYTOIN SODIUM:Anaphylactic reaction:0.29), (POLYETHYLENE GLYCOLS:Anaphylactic reaction:1.54), (PREDNISOLONE\PREDNISOLONE ACETATE\PREDNISOLONE SODIUM SUCCINATE:Anaphylactic reaction:0.27), (PROTRIPTYLINE HYDROCHLORIDE:Anaphylactic reaction:0.12), (PYRAZINAMIDE:Anaphylactic reaction:0.72), (SACUBITRIL\VALSARTAN:Anaphylactic reaction:0.16), (TERAZOSIN HYDROCHLORIDE:Anaphylactic reaction:0.08), (TERIFLUNOMIDE:Anaphylactic reaction:0.62), (THEOPHYLLINE:Anaphylactic reaction:0.46), (TOCOPHEROL:Anaphylactic reaction:0.3), (TOLTERODINE:Anaphylactic reaction:0.04), (UMECLIDINIUM BROMIDE:Anaphylactic reaction:0.54), (ZOPICLONE:Anaphylactic reaction:1.07), (PHENYTOIN:Anaplastic astrocytoma:0.04), (AMIODARONE HYDROCHLORIDE:Androgen deficiency:0.21), (CALCIUM CARBONATE\CHOLECALCIFEROL:Androgen deficiency:0.02), (CANDESARTAN CILEXETIL:Androgen deficiency:0.14), (CELECOXIB:Androgen deficiency:0.29), (CERTOLIZUMAB PEGOL:Androgen deficiency:0.1), (DEVICE:Androgen deficiency:0.16), (DEXLANSOPRAZOLE:Androgen deficiency:0.3), (ENOXAPARIN:Androgen deficiency:0.22), (FLECAINIDE:Androgen deficiency:0.02), (ILOPROST:Androgen deficiency:0.24), (LEDIPASVIR\SOFOSBUVIR:Androgen deficiency:0.03), (LOVASTATIN:Androgen deficiency:0.26), (MELOXICAM:Androgen deficiency:0.35), (OXCARBAZEPINE:Androgen deficiency:0.07), (POLYETHYLENE GLYCOLS:Androgen deficiency:0.02), (SACUBITRIL\VALSARTAN:Androgen deficiency:0.08), (TERIFLUNOMIDE:Androgen deficiency:0.15), (TIOTROPIUM BROMIDE MONOHYDRATE:Androgen deficiency:0.72), (TOPIRAMATE:Androgen deficiency:0.64), (UMECLIDINIUM BROMIDE\VILANTEROL TRIFENATATE:Androgen deficiency:0.05), (ACARBOSE:Androgen replacement therapy:0.18), (ACETAZOLAMIDE:Androgen replacement therapy:0.51), (ALBIGLUTIDE:Androgen replacement therapy:0.02), (ATROPINE SULFATE\DIPHENOXYLATE HYDROCHLORIDE:Androgen replacement therapy:0.51), (BENAZEPRIL\BENAZEPRIL HYDROCHLORIDE:Androgen replacement therapy:0.14), (BISACODYL:Androgen replacement therapy:0.3), (BRINZOLAMIDE:Androgen replacement therapy:0.14), (BUPRENORPHINE HYDROCHLORIDE:Androgen replacement therapy:0.3), (CANDESARTAN:Androgen replacement therapy:0.52), (CANDESARTAN CILEXETIL\HYDROCHLOROTHIAZIDE:Androgen replacement therapy:0.3), (CARVEDILOL PHOSPHATE:Androgen replacement therapy:1.01), (CERTOLIZUMAB PEGOL:Androgen replacement therapy:0.42), (CLINDAMYCIN\CLINDAMYCIN PHOSPHATE:Androgen replacement therapy:0.73), (CLOZAPINE:Androgen replacement therapy:0.02), (CORTISONE\HYDROCORTISONE:Androgen replacement therapy:0.08), (DABIGATRAN:Androgen replacement therapy:0.31), (DACLIZUMAB:Androgen replacement therapy:0.15), (DASATINIB:Androgen replacement therapy:0.21), (DEVICE:Androgen replacement therapy:0.6), (DIETARY SUPPLEMENT:Androgen replacement therapy:0.16), (DOFETILIDE:Androgen replacement therapy:0.36), (EDOXABAN TOSYLATE:Androgen replacement therapy:0.32), (ERYTHROPOIETIN:Androgen replacement therapy:0.26), (ESTROGENS, CONJUGATED:Androgen replacement therapy:0.46), (ESTROGENS, CONJUGATED\MEDROXYPROGESTERONE ACETATE:Androgen replacement therapy:0.13), (EVEROLIMUS:Androgen replacement therapy:0.05), (EVOLOCUMAB:Androgen replacement therapy:1.03), (FESOTERODINE FUMARATE:Androgen replacement therapy:0.66), (FISH OIL\OMEGA-3 FATTY ACIDS:Androgen replacement therapy:0.23), (FLUTICASONE FUROATE\VILANTEROL TRIFENATATE:Androgen replacement therapy:0.51), (GLICLAZIDE:Androgen replacement therapy:0.26), (GUAIFENESIN:Androgen replacement therapy:0.21), (HALOPERIDOL:Androgen replacement therapy:0.01), (HERBALS\TURMERIC:Androgen replacement therapy:0.02), (IBRUTINIB:Androgen replacement therapy:0.66), (ILOPROST:Androgen replacement therapy:0.3), (IMIPRAMINE HYDROCHLORIDE:Androgen replacement therapy:0.21), (INSULIN DEGLUDEC:Androgen replacement therapy:0.14), (LERCANIDIPINE HYDROCHLORIDE:Androgen replacement therapy:0.08), (LIDOCAINE HYDROCHLORIDE:Androgen replacement therapy:0.57), (LINACLOTIDE:Androgen replacement therapy:0.1), (LOMITAPIDE MESYLATE:Androgen replacement therapy:0.13), (METHIMAZOLE:Androgen replacement therapy:0.3), (METHYLPREDNISOLONE ACETATE:Androgen replacement therapy:0.5), (METHYLPREDNISOLONE SODIUM SUCCINATE:Androgen replacement therapy:0.32), (METRONIDAZOLEMETRONIDAZOLE HYDROCHLORIDE:Androgen replacement therapy:0.12), (NALTREXONE:Androgen replacement therapy:0.04), (NITROFURANTOIN:Androgen replacement therapy:0.28), (PANTOPRAZOLE MAGNESIUM:Androgen replacement therapy:0.64), (PEGINTERFERON BETA-1A:Androgen replacement therapy:0.1), (PENICILLIN:Androgen replacement therapy:0.6), (PHENOBARBITAL:Androgen replacement therapy:0.21), (PROBIOTICS NOS:Androgen replacement therapy:0.57), (PROPAFENONE HYDROCHLORIDE:Androgen replacement therapy:0.29), (PYRIDOXINE:Androgen replacement therapy:0.13), (PYRIDOXINE HYDROCHLORIDE:Androgen replacement therapy:0.17), (RALOXIFENE HYDROCHLORIDE:Androgen replacement therapy:0.32), (ROFLUMILAST:Androgen replacement therapy:0.29), (SODIUM CHLORIDE:Androgen replacement therapy:0.1), (SODIUM OXYBATE:Androgen replacement therapy:1.18), (SUMATRIPTAN\SUMATRIPTAN SUCCINATE:Androgen replacement therapy:0.81), (TERIFLUNOMIDE:Androgen replacement therapy:0.5), (TETRACYCLINE:Androgen replacement therapy:0.66), (UMECLIDINIUM BROMIDE\VILANTEROL TRIFENATATE:Androgen replacement therapy:0.81), (VILAZODONE HYDROCHLORIDE:Androgen replacement therapy:0.29), (ZOPICLONE:Androgen replacement therapy:0.23), (ALCOHOL:Anger:0.2), (ATORVASTATIN CALCIUM:Anger:0.67), (BUPRENORPHINE HYDROCHLORIDE:Anger:0.23), (CYCLOBENZAPRINE HYDROCHLORIDE:Anger:0.1), (ENALAPRIL:Anger:0.02), (EVOLOCUMAB:Anger:0.07), (N APROXEN:Anger:0.69), (PRAMIPEXOLE:Anger:0), (PRIMIDONE:Anger:0.22), (RALOXIFEN E HYDROCHLORIDE:Anger:0.01), (ZONISAMIDE:Anger:0.41), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Angina pectoris:0.07), (.ALPHA.-TOCOPHEROL\RETINOL:Angina pectoris:0.47), (.ALPHA.1-PROTEINASE INHIBITOR HUMAN:Angina pectoris:0.56), (ACETAMINOPHEN\APRONALIDE\CAFFEINE\PROPYPHENAZONE:Angin a pectoris:0.35), (ACETAMINOPHEN\DIPHENHYDRAMINE:Angina pectoris:0.21), (ACITRETIN:Angina pectoris:0.18), (ACLIDINIUM BROMIDE\FORMOTEROL FUMARATE:Angina pectoris:0.03), (ADENINE:Angina pectoris:1.02), (ADUCANUMAB:Angina pectoris:0.16), (AMLODIPINE BESYLATE\HYDROCHLOROTHIAZIDE\VALSARTAN:Angina pectoris:1.4), (AMLODIPINE\VALSARTAN:Angina pectoris:1.22), (AMPHETAMINE ASPARTATE\AMPHETAMINE SULFATE\DEXTROAMPHETAMINE SACCHARATE\DEXTROAMPHETAMINE SULFATE:Angina pectoris:1.84), (AMPHOTERICIN B:Angina pectoris:0.27), (ANTIHISTAMINES NOS:Angina pectoris:0.39), (ARIPIPRAZOLE LAUROXIL:Angina pectoris:0.05), (ATENOLOL\CHLORTHALIDONE:Angina pectons:0.54), (ATROPINE SULFATE:Angina pectoris:0.31), (ATROPINE\DIPHENOXYLATE:Angina pectoris:0.35), (BACITRACIN:Angina pectoris:0.06), (BECLOMETHASONE DIPROPIONATE\FORMOTEROL:Angina pectoris:0.12), (BENSERAZIDE\LEVODOPA:Angina pectoris:0.1), (BERAPROST:Angina pectoris:0.14), (BETAMETHASONE BUTYRATE PROPIONATE:Angina pectoris:0.29), (BETAMETHASONE SODIUM PHOSPHATE\NEOMYCIN SULFATE:Angina pectoris:0.38), (BETHANECHOL CHLORIDE:Angina pectoris:0.67), (BICARBONATE ION:Angina pectoris:0.1), (BILASTINE:Angina pectoris:0.63), (BIMATOPROST\TIMOLOL:Angina pectoris:0.18), (BROMHEXINE:Angina pectoris:0.01), (BUCOLOME:Angina pectoris:0.15), (BUFORMIN:Angina pectoris:0.52), (CALCITONIN:Angina pectoris:0.7), (CALCITONIN SALMON:Angina pectoris:1), (CALCIUM CARBONATE\ERGOCALCIFEROL\RETINOL:Angina pectoris:0.69), (CALCIUM CHLORIDE ANHYDROUS\POTASSIUM CHLORIDE\SODIUM CHLORIDE\SODIUM LACTATE:Angina pectoris:0.54), (CALCIUM LACTATE:Angina pectoris:0.78), (CALCIUM\MAGNESIUM:Angina pectoris:0.47), (CALCIUM\MAGNESIUM\ZINC:Angina pectoris:0.66), (CAMPHOR\MENTHOL\METHYL SALICYLATE:Angina pectoris:0.33), (CANAGLIFLOZIN\METFORMIN HYDROCHLORIDE:Angina pectoris:1.91), (CARBIDOPA\ENTACAPONE\LEVODOPA:Angina pectoris:0.01), (CARBOHYDRATES\POTASSIUM CHLORIDE\SODIUM CHLORIDE\SODIUM LACTATE:Angina pectoris:0.4), (CARTEOLOL HYDROCHLORIDE:Angina pectoris:0.55), (CASANTHRANOL\DOCUSATE SODIUM:Angina pectoris:0.31), (CELLULASE\DIASTASE\LIPASE:Angina pectoris:0.6), (CEPHARANTHINE:Angina pectoris:0.14), (CERITINIB:Angina pectoris:0.69), (CERTOPARIN SODIUM:Angina pectoris:0.25), (CETIRIZINE HYDROCHLORIDE\PSEUDOEPHEDRINE HYDROCHLORIDE:Angina pectoris:0.52), (CEVIMELINE HYDROCHLORIDE:Angina pectoris:0.2), (CHLORDIAZEPOXIDE HYDROCHLORIDE\CLIDINIUM BROMIDE:Angina pectoris:0.23), (CLENBUTEROL HYDROCHLORIDE:Angina pectoris:0.98), (CLOMIPRAMINE HYDROCHLORIDE:Angina pectoris:0.72), (CORTICOTROPIN:Angina pectoris:0.97), (CROTAMITON:Angina pectoris:0.19), (CYAMEMAZINE:Angina pectoris:0.01), (CYANOCOBALAMIN\LIDOCAINE HYDROCHLORIDE\PYRIDOXINE HYDROCHLORIDE\THIAMINE HYDROCHLORIDE:Angina pectoris:0.01), (DABRAFENIB:Angina pectoris:0.57), (DACLIZUMAB:Angina pectoris:0.63), (DANAZOL:Angina pectoris:1.31), (DANTROLENE SODIUM:Angina pectoris:0.15), (DAPAGLIFLOZIN PROPANEDIOL\METFORMIN HYDROCHLORIDE:Angina pectoris:0.38), (DAPSONE:Angina pectoris:0.58), (DASABUVIR\OMBITASVIR\PARITAPREVIR\RITONAVIR:Angina pectoris:0.48), (DASATINIB:Angina pectoris:2.75), (DESMOPRESSIN:Angina pectoris:0.11), (DEXMEDETOMIDINE HYDROCHLORIDE:Angina pectoris:0.05), (DEXTROAMPHETAMINE SULFATE:Angina pectoris:0.34), (DICLOFENAC SODIUM\MISOPROSTOL:Angina pectoris:0.13), (DIFLUCORTOLONE VALERATE\LIDOCAINE:Angina pectoris:0.11), (DIFLUPREDNATE:Angina pectoris:0.14), (DIGITALIS:Angina pectoris:0.22), (DIGITOXIN:Angina pectoris:1.8), (DIMEMORFAN PHOSPHATE:Angina pectoris:0.13), (DIPYRONE:Angina pectoris:0.35), (DOLASETRON MESYLATE:Angina pectoris:0.17), (DOXYCYCLINE HYCLATE:Angina pectoris:0.21), (DOXYLAMINE SUCCINATE:Angina pectoris:0.27), (DRONABINOL:Angina pectoris:1.28), (DUTASTERIDE\TAMSULOSIN HYDROCHLORIDE:Angina pectoris:1.49), (ECONAZOLE:Angina pectoris:1.37), (ELTROMBOPAG OLAMINE:Angina pectoris:0.18), (EMPAGLIFLOZIN\LINAGLIPTIN:Angina pectoris:1.16), (ENALAPRIL MALEATE\LERCANIDIPINE HYDROCHLORIDE:Angina pectoris:0.23), (ENTACAPONE:Angina pectoris:0.16), (EPIRUBICIN:Angina pectoris:0.77), (ERIBULIN:Angina pectoris:0.15), (ESTROGENS, CONJUGATED\MEDROXYPROGESTERONE ACETATE:Angina pectoris:0.75), (ETHACRYNIC ACID:Angina pectoris:0.35), (ETRETINATE:Angina pectoris:1.4), (FAROPENEM SODIUM:Angina pectoris:0.39), (FENOTEROL HYDROBROMIDE:Angina pectoris:0.99), (FERROUS GLYCINE SULFATE:Angina pectoris:0.22), (FERROUS SULFATE\FOLIC ACID:Angina pectoris:0.4), (FEXOFENADINE\PSEUDOEPHEDRINE:Angina pectoris:0.41), (FINGOLIMOD HYDROCHLORIDE:Angina pectoris:0.88), (FLAVIN ADENINE DINUCLEOTIDE:Angina pectoris:0.58), (FLUTAMIDE:Angina pectoris:0.11), (FOLATE SODIUM:Angina pectoris:0.31), (FORMOTEROL FUMARATE\MOMETASONE FUROATE:Angina pectoris:0.79), (FOSFOMYCIN CALCIUM:Angina pectoris:0.18), (FUDOSTEINE:Angina pectoris:0.31), (FURSULTIAMINE:Angina pectoris:1.43), (GLIMEPIRIDE\ROSIGLITAZONE MALEATE:Angina pectoris:0.8), (GLIPIZIDE\METFORMIN HYDROCHLORIDE:Angina pectoris:0.21), (GLYCOPYRROLATE\INDACATEROL MALEATE:Angina pectoris:0.65), (GUANABENZ ACETATE:Angina pectoris:1.21), (GUANFACINE:Angina pectoris:0.84), (HYOSCYAMINE:Angina pectoris:0.9), (IFENPRODIL:Angina pectoris:0.36), (IGURATIMOD:Angina pectoris:0.32), (IMIGLUCERASE:Angina pectoris:0.45), (INSULIN ASPART\INSULIN DEGLUDEC:Angina pectoris:1.01), (INSULIN PORK\INSULIN PURIFIED PORK:Angina pectoris:0.24), (INTERFERON:Angina pectoris:0.3), (INTERFERON ALFA-2A:Angina pectoris:0.47), (INTERFERON ALFA-2B:Angina pectoris:0.53), (IPRAGLIFLOZIN:Angina pectoris:0.13), (ISOLEUCINE\LEUCINE\VALINE:Angina pectoris:0), (LABETALOL HYDROCHLORIDE:Angina pectoris:1.2), (LACTOBACILLUS RHAMNOSUS:Angina pectoris:0.71), (LANDIOLOL HYDROCHLORIDE:Angina pectoris:0.45), (LANREOTIDE:Angina pectoris:0.25), (LAPATINIB DITOSYLATE:Angina pectoris:0.59), (LENVATINIB:Angina pectoris:0.35), (LEVOCABASTINE HYDROCHLORIDE:Angina pectoris:0.88), (LICORICE ROOT EXTRACT\RHUBARB:Angina pectoris:0.19), (LIOTHYRONINE:Angina pectoris:0.3), (LORNOXICAM:Angina pectoris:0.43), (LOTEPREDNOL ETABONATE:Angina pectoris:0.34), (LULICONAZOLE:Angina pectoris:0.28), (LURASIDONE HYDROCHLORIDE:Angina pectoris:0.22), (LUTETIUM LU-177:Angina pectoris:0.15), (LYSOZYME HYDROCHLORIDE:Angina pectoris:0.29), (MAGNESIUM ASPARTATE HYDROCHLORIDE:Angina pectoris:0.23), (MEFENAMIC ACID:Angina pectoris:0.34), (MEGESTROL:Angina pectoris:0.08), (MEPENZOLATE BROMIDE:Angina pectoris:0.28), (MEPERIDINE HYDROCHLORIDE:Angina pectoris:0.53), (MEPOLIZUMAB:Angina pectoris:0.81), (METFORMIN HYDROCHLORIDE\ROSIGLITAZONE MALEATE:Angina pectoris:1.12), (METFORMIN HYDROCHLORIDE\SAXAGLIPTIN:Angina pectoris:0.33), (METFORMIN HYDROCHLORIDE\SAXAGLIPTIN HYDROCHLORIDE:Angina pectoris:0.55), (METFORMIN\SITAGLIPTIN:Angina pectoris:0.22), (METRELEPTIN:Angina pectoris:0.42), (MORPHINE SULFATE\NALTREXONE HYDROCHLORIDE:Angina pectoris:0.06), (NADROPARIN CALCIUM:Angina pectoris:1.52), (NARATRIPTAN HYDROCHLORIDE:Angina pectoris:0.02), (NIRAPARIB:Angina pectoris:0.72), (NITROFURANTOIN\NITROFURANTOIN MONOHYDRATE:Angina pectoris:0.49), (NOREPINEPHRINE:Angina pectoris:0.36), (NORETHINDRONE:Angina pectoris:0.15), (OLAPARIB:Angina pectoris:0.26), (ORPHENADRINE:Angina pectoris:0.14), (OSELTAMIVIR:Angina pectoris:0.23), (OXYBUTYNIN\OXYBUTYNIN CHLORIDE:Angina pectoris:0.42), (OXYMETAZOLINE:Angina pectoris:0.25), (OZAGREL:Angina pectoris:0.04), (PANCRELIPASE AMYLASE\PANCRELIPASE LIPASE\PANCRELIPASE PROTEASE:Angina pectoris:0.63), (PEGINTERFERON BETA-1A:Angina pectoris:1.27), (PENICILLIN:Angina pectoris:1.52), (PENICILLIN G:Angina pectoris:0.35), (PHLOROGLUCINOL\1,3,5-TRIMETHOXYBENZENE:Angina pectoris:0.16), (PIMECROLIMUS:Angina pectoris:0.21), (PIPERACILLIN:Angina pectoris:0.15), (POLYETHYLENE GLYCOL 3350\POTASSIUM CHLORIDE\SODIUM BICARBONATE\SODIUM CHLORIDE\SODIUM SULFATE ANHYDROUS:Angina pectoris:0.5), (POTASSIUM CHLORIDE\SODIUM CHLORIDE\SODIUM LACTATE:Angina pectoris:0.25), (PRAZEPAM:Angina pectoris:0.07), (PROPIVERINE:Angina pectoris:0.57), (PYRIDOXINE:Angina pectoris:0.28), (RADIATION THERAPY:Angina pectoris:0.35), (RILPIVIRINE HYDROCHLORIDE:Angina pectoris:0.42), (RIZATRIPTAN BENZOATE:Angina pectoris:1.1), (ROSIGLITAZONE:Angina pectoris:0.23), (ROXITHROMYCIN:Angina pectoris:0.03), (SELEXIPAG:Angina pectoris:0.69), (SIPULEUCEL-T:Angina pectoris:0.06), (SIVELESTAT SODIUM:Angina pectoris:0.11), (SODIUM CITRATE:Angina pectoris:0.15), (SODIUM PHOSPHATE, DIBASIC\SODIUM PHOSPHATE, MONOBASIC:Angina pectoris:0.7), (SOFALCONE:Angina pectoris:0.2), (SOFOSBUVIR\VELPATASVIR:Angina pectoris:0.37), (SUMATRIPTAN\SUMATRIPTAN SUCCINATE:Angina pectoris:1.37), (SUPLATAST TOSILATE:Angina pectoris:0.88), (TAFLUPROST:Angina pectoris:0.26), (TEDUGLUTIDE\WATER:Angina pectoris:0.08), (TELOTRISTAT ETHYL:Angina pectoris:0.1), (TEMSIROLIMUS:Angina pectoris:0.15), (TIMOLOL MALEATE\TRAVOPROST:Angina pectoris:0.32), (TIMOLOL\TRAVOPROST:Angina pectoris:0.14), (TIPEPIDINE HIBENZATE:Angina pectoris:0.44), (TIZANIDINE HYDROCHLORIDE:Angina pectoris:1.52), (TOBRAMYCIN:Angina pectoris:0.43), (TRAMETINIB:Angina pectoris:0.53), (TREPROSTINIL DIOLAMINE:Angina pectoris:0.02), (TRIHEXYPHENIDYL:Angina pectoris:0.14), (TRIMIPRAMINE MALEATE:Angina pectoris:0.35), (UMECLIDINIUM BROMIDE:Angina pectoris:0.76), (UROKINASE:Angina pectoris:0.82), (VASOPRESSIN:Angina pectoris:0.32), (VINCRISTINE SULFATE:Angina pectoris:0.33), (VINORELBINE TARTRATE:Angina pectoris:0.03), (VONOPRAZAN:Angina pectoris:0.4), (XIPAMIDE:Angina pectoris:0.29), (ZILEUTON:Angina pectoris:0.56), (ZOLMITRIPTAN:Angina pectoris:0.12), (ZOTEPINE:Angina pectoris:0.06), (ACENOCOUMAROL:Angina unstable:0), (CALCIUM:Angina unstable:0.44), (CALCIUM CARBONATE\CHOLECALCIFEROL:Angina unstable:0.49), (COLCHICINE:Angina unstable:0.17), (DASATINIB:Angina unstable:0.08), (DICLOFENAC:Angina unstable:0.1), (FERROUS SULFATE\FERROUS SULFATE, DRIED:Angina unstable:0.4), (FLECAINIDE:Angina unstable:0.48), (FLUTICASONE PROPIONATE:Angina unstable:0.31), (HYDROCHLOROTHIAZIDE\LISINOPRIL:Angina unstable:0.04), (INSULIN LISPRO:Angina unstable:0.65), (LEDIPASVIR\SOFOSBUVIR:Angina unstable:0.13), (LOVASTATIN:Angina unstable:0.76), (OXYGEN:Angina unstable:0.43), (POTASSIUM:Angina unstable:0.75), (TRAMADOL:Angina unstable:0.44), (DILTIAZEM HYDROCHLORIDE:Angioedema:0.5), (FERROUS SULFATE\FERROUS SULFATE, DRIED:Angioedema:0.03), (METRONIDAZOLE:Angioedema:0.37), (ROSUVASTATIN CALCIUM:Angioedema:0.07), (VERAPAMIL HYDROCHLORIDE:Angioedema:0.08), (BRIVARACETAM:Angiomyolipoma:0.33), (DEXLA NSOPRAZOLE:Angiomyolipoma:0.23), (ENALAPRIL:Angiomyolipoma:0.07), (LOVASTATIN:Angiomyolipoma:0.07), (SODIUM OXYBATE:Angiomyolipoma:0.13), (VARENICLINE TARTRATE:Angiomyolipoma:0.06), (ACENOCOUMAROL:Angiopathy:0.12), (ANASTROZO LE:Angiopathy:0.44), (BISACODYL OR DOCUSATE SODIUM:Angiopathy:0.08), (CALCIUM ACETATE:Angiopathy:0.33), (CANDESARTAN CILEXETIL\HYDROCHLOROTHIAZIDE:Angiopathy:0.28), (CERTOLIZUMAB PEGOL:Angiopathy:0.09), (CLINDAMYCIN\CLINDAMYCIN PHOSPHATE:Angiopathy:0.54), (CLOTRIMAZOLE:Angiopathy:0.05), (DASATINIB:Angiopathy: 0.09), (DICLOFENAC SODIUM:Angiopathy:0.44), (ENALAPRIL MALEATE:Angiopathy:0.31), (EPLERENONE:Angiopathy:0.03), (ERYTHROPOIETIN:Angiopathy: 0.51), (EVOLOCUMAB:Angiopathy:0.72), (FERROUS SULFATE\FERROUS SULFATE, DRIED:Angiopathy:0.69), (FLUTICASONE FUROATE\VILANTEROL TRIFENATATE:Angiopathy:0.11), (GLICLAZIDE:Angiopathy:0.09), (HYDROMORPHONE HYDROCHLORIDE:Angiopathy:0.06), (IBRUTINIB:Angiopathy:0.78), (INDAPAMIDE:Angiopathy: 0.08), (INDAPAMIDE\PERINDOPRIL:Angiopathy:0.04), (LEDIPASVIR\SOFOSBUVIR: Angiopathy:0.06), (LERCANIDIPINE HYDROCHLORIDE:Angiopathy:0.16), (LEVALBUTEROL HYDROCHLORIDE:Angiopathy:0.01), (MELATONIN:Angiopathy:0.09), (METRONIDAZOL E:Angiopathy:0.41), (NAPROXEN SODIUM:Angiopathy:0.01), (NILOTINIB:Angiopathy:0.02), (NITROFURANTOIN:Angiopathy: 0.1), (OLANZAPINE:Angiopathy:0.06), (OMEGA-3-ACID ETHYL ESTERS:Angiopathy:0.33), (PAROXETINE\PAROXETINE HYDROCHLORIDE:Angiopathy:0.17), (PHENYTOIN:Angiopathy:0.11), (PRAMIPEXOLE DIHYDROCHLORIDE:Angiopathy:0.2), (PROMETHAZINE\PROMETHAZINE HYDROCHLORIDE:Angiopathy:0.1), (RABEPRAZOLE SODIUM:Angiopathy:0.26), (RALOXIFENE HYDROCHLORIDE:Angiopathy:0.02), (RIOCIGUAT:Angiopathy:0.89), (SACUBITRIL\VALS ARTAN:Angiopathy:0.4), (SODIUM OXYBATE:Angiopathy:0.03), (SOTALOL:Angiopathy:0.31), (TRIAMCINOLONE ACETONIDE:Angiopathy:0.21), (IBRUTINIB:Angioplasty:0.47), (NAPROXEN:Angioplasty:0.19), (.ALPHA.1-PROTEINASE INHIBITOR HUMAN:Ankylosing spondylitis:0.28), (ABOBOTULINUMTOXINA:Ankylosing spondylitis:0.42), (AGALSIDASE BETA:Ankylosing spondylitis:0.84), (ALBUTEROL\IPRATROPIUM:Ankylosing spondylitis:0.01), (AMANTADINE:Ankylosing spondylitis:1.26), (AMMONIUM LACTATE:Ankylosing spondylitis:0.93), (ANAKINRA:Ankylosing spondylitis:0.91), (ANASTROZOLE:Ankylosing spondylitis:1.5), (ANTIHISTAMINES NOS:Ankylosing spondylitis:0.41), (ASCORBIC ACID/CHROMIUM/SELENIUM/VANADYL SULFATE:Ankylosing spondylitis:0.28), (ASPIRIN\BUTALBITAL\CAFFEINE:Ankylosing spondylitis:0.06), (ATROPINE\DIPHENOXYLATE:Ankylosing spondylitis:0.2), (AZILSARTAN KAMEDOXOMIL:Ankylosing spondylitis:0.65), (BETAMETHASONE DIPROPIONATE\CALCIPOTRIENE:Ankylosing spondylitis:0.35), (BETAMETHASONE DIPROPIONATE\CALCIPOTRIENE HYDRATE:Ankylosing spondylitis:0.16), (BETHANECHOL CHLORIDE:Ankylosing spondylitis:0.24), (BIMATOPROST\TIMOLOL:Ankylosing spondylitis:0.11), (BOTULINUM TOXIN TYPE A:Ankylosing spondylitis:0.4), (BROMFENAC SODIUM:Ankylosing spondylitis:0.1), (BUPIVACAINE HYDROCHLORIDE:Ankylosing spondylitis:0.27), (CALCITONIN SALMON:Ankylosing spondylitis:1.24), (CALCIUM CITRATE\VITAMIN D:Ankylosing spondylitis:0.14), (CALCIUM PANTOTHENATE\CYANOCOBALAMIN\NIACINAMIDE\PYRIDOXINE HYDROCHLORIDE\RIBOFLAVIN\THIAMINE MONONITRATE:Ankylosing spondylitis:0.09), (CALCIUM\MAGNESIUM\ZINC:Ankylosing spondylitis:0.97), (CANAGLIFLOZIN\METFORMIN HYDROCHLORIDE:Ankylosing spondylitis:0.69), (CARBIMAZOLE:Ankylosing spondylitis:0.27), (CARFILZOMIB:Ankylosing spondylitis:0.57), (CARTEOLOL HYDROCHLORIDE:Ankylosing spondylitis:0.5), (CARVEDILOL PHOSPHATE:Ankylosing spondylitis:2.01), (CASANTHRANOL\DOCUSATE SODIUM:Ankylosing spondylitis:0.35), (CEFAZOLIN:Ankylosing spondylitis:0.13), (CEFPODOXIME PROXETIL:Ankylosing spondylitis:0.23), (CERIVASTATIN SODIUM:Ankylosing spondylitis:0.18), (CHLORPHENIRAMINE\HYDROCODONE:Ankylosing spondylitis:0.08), (CILASTATIN SODIUM\IMIPENEM:Ankylosing spondylitis:0.53), (CLIKSTAR:Ankylosing spondylitis:0.49), (CONVALLARIA MAJALIS\CRATAEGUS LAEVIGATA LEAF\PROXYPHYLLINE:Ankylosing spondylitis:0.24), (DABIGATRAN:Ankylosing spondylitis:0.68), (DACLIZUMAB:Ankylosing spondylitis:1.08), (DANAZOL:Ankylosing spondylitis:0.76), (DASATINIB:Ankylosing spondylitis:1.01), (DEXAMETHASONE ACETATE:Ankylosing spondylitis:0.02), (DIGITOXIN:Ankylosing spondylitis:0.28), (EDOXABAN TOSYLATE:Ankylosing spondylitis:1.01), (ERTAPENEM SODIUM:Ankylosing spondylitis:0.01), (ETHACRYNIC ACID:Ankylosing spondylitis:0.19), (EVEROLIMUS:Ankylosing spondylitis:0.69), (FENOTEROL HYDROBROMIDE:Ankylosing spondylitis:1.14), (FERROUS BISGLYCINATE:Ankylosing spondylitis:0.74), (FERROUS SULFATE\GLYCINE:Ankylosing spondylitis:0.9), (FLUNARIZINE HYDROCHLORIDE:Ankylosing spondylitis:0.02), (FLUOCINOLONE ACETONIDE:Ankylosing spondylitis:1.04), (FONDAPARINUX:Ankylosing spondylitis:0.13), (FONDAPARINUX SODIUM:Ankylosing spondylitis:0.01), (FORMOTEROL FUMARATE\MOMETASONE FUROATE:Ankylosing spondylitis:1.14), (GLYCOPYRROLATE\INDACATEROL MALEATE:Ankylosing spondylitis:0.55), (GLYCOPYRRONIUM\INDACATEROL:Ankylosing spondylitis:1.33), (GOLD SODIUM THIOMALATE:Ankylosing spondylitis:0.04), (HEPARIN CALCIUM:Ankylosing spondylitis:0.72), (HORSE CHESTNUT:Ankylosing spondylitis:0.16), (HYDROCHLOROTHIAZIDE\RAMIPRIL:Ankylosing spondylitis:0.06), (HYDROCORTISONE ACETATE:Ankylosing spondylitis:1.73), (IBRUTINIB:Ankylosing spondylitis:0.64), (IMATINIB MESYLATE:Ankylosing spondylitis:0.54), (INSULIN DEGLUDEC:Ankylosing spondylitis:0.59), (INSULIN PORK\INSULIN PURIFIED PORK:Ankylosing spondylitis:0.08), (INTERFERON ALFA-2A:Ankylosing spondylitis:0.32), (IPRATROPIUM:Ankylosing spondylitis:1.82), (IXAZOMIB:Ankylosing spondylitis:0.19), (LABETALOL HYDROCHLORIDE:Ankylosing spondylitis:1.96), (LEDIPASVIR\SOFOSBUVIR:Ankylosing spondylitis:0.65), (LEVALBUTEROL:Ankylosing spondylitis:0.16), (LEVOMILNACIPRAN HYDROCHLORIDE:Ankylosing spondylitis:0.02), (LIDOCAINE\PRILOCAINE:Ankylosing spondylitis:0.49), (LOMITAPIDE MESYLATE:Ankylosing spondylitis:0.35), (MAGNESIUM ASPARTATE HYDROCHLORIDE:Ankylosing spondylitis:0.37), (MEDROXYPROGESTERONE:Ankylosing spondylitis:0.41), (MELPERONE:Ankylosing spondylitis:0.05), (MEMANTINE HYDROCHLORIDE:Ankylosing spondylitis:0.4), (METFORMIN HYDROCHLORIDE\VILDAGLIPTIN:Ankylosing spondylitis:0.62), (METHENAMINE HIPPURATE:Ankylosing spondylitis:1.1), (METRELEPTIN:Ankylosing spondylitis:0.42), (MICONAZOLE NITRATE:Ankylosing spondylitis:0.52), (MIDODRINE HYDROCHLORIDE:Ankylosing spondylitis:0.23), (NALBUPHINE HYDROCHLORIDE:Ankylosing spondylitis:0.15), (NICARDIPINE:Ankylosing spondylitis:0.43), (NICORANDIL:Ankylosing spondylitis:0.08), (NILOTINIB:Ankylosing spondylitis:1.13), (OXYMETAZOLINE:Ankylosing spondylitis:0.23), (PALIPERIDONE PALMITATE:Ankylosing spondylitis:0.31), (PAZOPANIB HYDROCHLORIDE:Ankylosing spondylitis:0.47), (PEGINTERFERON BETA-1A:Ankylosing spondylitis:0.84), (PEMETREXED DISODIUM:Ankylosing spondylitis:0.23), (PENICILLIN G:Ankylosing spondylitis:0.52), (PENICILLIN V:Ankylosing spondylitis:0.24), (PHENYTOIN SODIUM:Ankylosing spondylitis:1.02), (PONATINIB:Ankylosing spondylitis:0.39), (POVIDONE:Ankylosing spondylitis:0.11), (PRAZOSIN HYDROCHLORIDE:Ankylosing spondylitis:0.65), (PREDNISOLONE\PREDNISOLONE ACETATE\PREDNISOLONE SODIUM SUCCINATE:Ankylosing spondylitis:0.7), (PROTRIPTYLINE HYDROCHLORIDE:Ankylosing spondylitis:0.47), (RETINOL:Ankylosing spondylitis:0.57), (RILMENIDINE PHOSPHATE:Ankylosing spondylitis:0.54), (RIVASTIGMINE:Ankylosing spondylitis:0.74), (RIVASTIGMINE TARTRATE:Ankylosing spondylitis:0.95), (SALMETEROL:Ankylosing spondylitis:0.67), (SALMETEROL XINAFOATE:Ankylosing spondylitis:0.64), (SELEGILINE:Ankylosing spondylitis:0.26), (SIBUTRAMINE HYDROCHLORIDE:Ankylosing spondylitis:0.32), (SITAGLIPTIN:Ankylosing spondylitis:0.84), (SODIUM PHOSPHATE, DIBASIC\SODIUM PHOSPHATE, MONOBASIC:Ankylosing spondylitis:0.1), (SUNITINIB MALATE:Ankylosing spondylitis:0.22), (TERBUTALINE:Ankylosing spondylitis:0.72), (TERIFLUNOMIDE:Ankylosing spondylitis:0.67), (TESTOSTERONE UNDECANOATE:Ankylosing spondylitis:0.93), (TETRACYCLINE:Ankylosing spondylitis:1.86), (THEOPHYLLINE:Ankylosing spondylitis:0.09), (THIOCTIC ACID:Ankylosing spondylitis:0.22), (TINZAPARIN SODIUM:Ankylosing spondylitis:0.44), (TOFACITINIB:Ankylosing spondylitis:0.8), (TRIHEXYPHENIDYL:Ankylosing spondylitis:0.04), (TRIMIPRAMINE MALEATE:Ankylosing spondylitis:0.77), (TRIPTORELIN PAMOATE:Ankylosing spondylitis:0.01), (UMECLIDINIUM BROMIDE:Ankylosing spondylitis:0.5), (VALDECOXIB:Ankylosing spondylitis:0.49), (VALGANCICLOVIR HYDROCHLORIDE:Ankylosing spondylitis:0.51), (ZILEUTON:Ankylosing spondylitis:0.34), (ACETAMINOPHEN\TRAMADOL:Antacid therapy:0), (ADO-TRASTUZUMAB EMTANSINE:Antacid therapy:0.66), (ALISKIREN HEMIFUMARATE:Antacid therapy:0.21), (AMIKACIN SULFATE:Antacid therapy:0), (AMISULPRIDE:Antacid therapy:0.04), (AZELASTINE:Antacid therapy:0), (BENAZEPRIL\BENAZEPRIL HYDROCHLORIDE:Antacid therapy:0.37), (BLINATUMOMAB:Antacid therapy:0.26), (BRINZOLAMIDE:Antacid therapy:0.11), (BUDESONIDE\FORMOTEROL FUMARATE:Antacid therapy:0.17), (BUPRENORPHINE HYDROCHLORIDE:Antacid therapy:0.05), (BUPRENORPHINE HYDROCHLORIDE\NALOXONE HYDROCHLORIDE:Antacid therapy:0.08), (CANAKINUMAB:Antacid therapy:0.24), (CARVEDILOL PHOSPHATE:Antacid therapy:0.59), (CEFOTAXIME SODIUM:Antacid therapy:0.47), (CHLORTHALIDONE:Antacid therapy:0.36), (CINACALCET HYDROCHLORIDE:Antacid therapy:0.01), (CLONIDINE HYDROCHLORIDE:Antacid therapy:0.1), (DACLIZUMAB:Antacid therapy:0.79), (DANAZOL:Antacid therapy:0.22), (DAPAGLIFLOZIN PROPANEDIOL:Antacid therapy:0.56), (DAPSONE:Antacid therapy:0.24), (DESVENLAFAXINE SUCCINATE:Antacid therapy:0.24), (DIPYRONE:Antacid therapy:0.18), (DOXEPIN HYDROCHLORIDE:Antacid therapy:0.07), (ELETRIPTAN HYDROBROMIDE:Antacid therapy:0.59), (ELOTUZUMAB:Antacid therapy:0.4), (EMPAGLIFLOZIN\LINAGLIPTIN:Antacid therapy:0.54), (ENALAPRIL MALEATE:Antacid therapy:0.94), (ESTROGENS, CONJUGATED\MEDROXYPROGESTERONE ACETATE:Antacid therapy:0), (EVOLOCUMAB:Antacid therapy:1.22), (FLUDROCORTISONE ACETATE:Antacid therapy:0.06), (FLUTICASONE FUROATE:Antacid therapy:0.19), (FORMOTEROL FUMARATE:Antacid therapy:0.11), (FUSIDIC ACID:Antacid therapy:0.14), (GLYCOPYRRONIUM\lNDACATEROL:Antacid therapy:0.28), (HERBALS\TURMERIC:Antacid therapy:0.27), (HYDROCHLOROTHIAZIDE\IRBESARTAN:Antacid therapy:0.44), (HYDROCHLOROTHIAZIDE\TELMISARTAN:Antacid therapy:0.16), (HYDROCORTISONE ACETATE:Antacid therapy:0), (IMIPRAMINE HYDROCHLORIDE:Antacid therapy:0.35), (INDAPAMIDE\PERINDOPRIL:Antacid therapy:0.64), (INSULIN DEGLUDEC:Antacid therapy:0.18), (IRINOTECAN HYDROCHLORIDE:Antacid therapy:0.19), (ITRACONAZOLE:Antacid therapy:0.14), (LACTOBACILLUS RHAMNOSUS:Antacid therapy:0.03), (LAPATINIB DITOSYLATE:Antacid therapy:0.05), (LEDIPASVIR\SOFOSBUVIR:Antacid therapy:0.79), (LERCANIDIPINE:Antacid therapy:1.04), (LERCANIDIPINE HYDROCHLORIDE:Antacid therapy:0.42), (LIDOCAINE\PRILOCAINE:Antacid therapy:0.56), (LITHIUM CARBONATE:Antacid therapy:0.59), (LOMITAPIDE MESYLATE:Antacid therapy:0.2), (MEGESTROL:Antacid therapy:0.02), (MEPERIDINE HYDROCHLORIDE:Antacid therapy:0.07), (METFORMIN HYDROCHLORIDE\VILDAGLIPTIN:Antacid therapy:0.21), (METHENAMINE HIPPURATE:Antacid therapy:0.62), (METHIMAZOLE:Antacid therapy:0.02), (METHYLPREDNISOLONE ACETATE:Antacid therapy:0.47), (MINOCYCLINE\MINOCYCLINE HYDROCHLORIDE:Antacid therapy:0.28), (NABUMETONE:Antacid therapy:0.11), (NALOXONE HYDROCHLORIDE\OXYCODONE HYDROCHLORIDE:Antacid therapy:0.68), (NINTEDANIB:Antacid therapy:0.22), (NIRAPARIB:Antacid therapy:0.14), (NOREPINEPHRINE BITARTRATE:Antacid therapy:0.04), (OLAPARIB:Antacid therapy:0.06), (OLODATEROL HYDROCHLORIDE\TIOTROPIUM BROMIDE MONOHYDRATE:Antacid therapy:0.11), (OMEPRAZOLE SODIUM:Antacid therapy:0.19), (PANTOPRAZOLE MAGNESIUM:Antacid therapy:0.7), (PEGINTERFERON BETA-1A:Antacid therapy:0.24), (PEMETREXED:Antacid therapy:0.34), (PENICILLIN:Antacid therapy:0.82), (PHENTOLAMINE MESYLATE:Antacid therapy:0.24), (PHENYTOIN SODIUM:Antacid therapy:0.11), (PHOSPHOCREATINE SODIUM:Antacid therapy:0.81), (PIROXICAM:Antacid therapy:0.24), (PRAZOSIN HYDROCHLORIDE:Antacid therapy:0.02), (PREDNISOLONE SODIUM PHOSPHATE:Antacid therapy:0.08), (PREDNISOLONE\PREDNISOLONE ACETATE\PREDNISOLONE SODIUM SUCCINATE:Antacid therapy:0.21), (QUETIAPINE:Antacid therapy:0.8), (RASBURICASE:Antacid therapy:0.06), (REGORAFENIB:Antacid therapy:0.17), (RIZATRIPTAN BENZOATE:Antacid therapy:0.45), (SCOPOLAMINE:Antacid therapy:0.67), (SEVELAMER CARBONATE:Antacid therapy:0.11), (SUMATRIPTAN:Antacid therapy:0.29), (SUMATRIPTAN\SUMATRIPTAN SUCCINATE:Antacid therapy:0.54), (TERBINAFINE HYDROCHLORIDE:Antacid therapy:0.38), (TETRACYCLINE:Antacid therapy:0.7), (THYROID, PORCINE:Antacid therapy:0.03), (TIMOLOL:Antacid therapy:0.41), (TIOTROPIUM:Antacid therapy:0.43), (UMECLIDINIUM BROMIDE\VILANTEROL TRIFENATATE:Antacid therapy:0.53), (VINORELBINE TARTRATE:Antacid therapy:0.11), (VITAMIN A:Antacid therapy:0.22), (ZINC:Antacid therapy:0.33), (ZONISAMIDE:Antacid therapy:0.26), (ALFACALCIDOL:Anti-infective therapy:0.05), (AMPICILLIN:Anti-infective therapy:0.54), (BENDAMUSTINE:Anti-infective therapy:0.16), (BLESELUMAB:Anti-infective therapy:0.09), (BLINATUMOMAB:Anti-infective therapy:0.62), (BUPROPION HYDROCHLORIDE:Anti-infective therapy:0.32), (CALCIUM:Anti-infective therapy:0.58), (CALCIUM ACETATE:Anti-infective therapy:0.04), (CANDESARTAN:Anti-infective therapy:0.13), (CANDESARTAN CILEXETIL:Anti-infective therapy:0.06), (CEFAZOLIN:Anti-infective therapy:0.22), (CHLORAMBUCIL:Anti-infective therapy:0.01), (CLINDAMYCIN\CLINDAMYCIN PHOSPHATE:Anti-infective therapy:1.2), (DASATINIB:Anti-infective therapy:0.27), (DEXLANSOPRAZOLE:Anti-infective therapy:0.12), (DIPHENHYDRAMINE:Anti-infective therapy:0.37), (ELOTUZUMAB:Anti-infective therapy:0.24), (EPINEPHRINE:Anti-infective therapy:0.27), (ETIMICIN SULFATE:Anti-infective therapy:0.36), (EVEROLIMUS:Anti-infective therapy:0.23), (FERROUS SULFATE:Anti-infective therapy:0.15), (FERROUS SULFATE\FERROUS SULFATE, DRIED:Anti-infective therapy:0.64), (IBRITUMOMAB TIUXETAN:Anti-infective therapy:0.4), (LEUCOGEN:Anti-infective therapy:0.38), (LEVOFLOXACIN HYDROCHLORIDE:Anti-infective therapy:0.29), (METHENOLONE ENANTHATE:Anti-infective therapy:0.1), (NAPROXEN:Anti-infective therapy:0.12), (NILOTINIB:Anti-infective therapy:0.51), (POTASSIUM:Anti-infective therapy:0.07), (PREDNISOLONE\PREDNISOLONE ACETATE\PREDNISOLONE SODIUM SUCCINATE:Anti-infective therapy:0.14), (SACUBITRIL\VALSARTAN:Anti-infective therapy:0.16), (SERTRALINE HYDROCHLORIDE:Anti-infective therapy:0.27), (SULBACTAM SODIUM:Anti-infective therapy:0.01), (THALIDOMIDE:Anti-infective therapy:0.45), (VINCRISTINE SULFATE:Anti-infective therapy:0.44), (ACETAMINOPHEN\BUTALBITAL\CAFFEINE:Antiallergic therapy:0.85), (ACLIDINIUM BROMIDE:Antiallergic therapy:0.16), (ADO-TRASTUZUMAB EMTANSINE:Antiallergic therapy:0.14), (AMPHETAMINE ASPARTATE\AMPHETAMINE SULFATE\DEXTROAMPHETAMINE SACCHARATE\DEXTROAMPHETAMINE SULFATE:Antiallergic therapy:0.62), (CALCITONIN SALMON:Antiallergic therapy:0.2), (CALCIUM CARBONATE\VITAMIN D:Antiallergic therapy:0.35), (CANDESARTAN CILEXETIL:Antiallergic therapy:0.75), (CANDESARTAN CILEXETIL\HYDROCHLOROTHIAZIDE:Antiallergic therapy:0.35), (CARFILZOMIB:Antiallergic therapy:0.67), (CARISOPRODOL:Antiallergic therapy:0.14), (CARVEDILOL PHOSPHATE:Antiallergic therapy:0.28), (CEFOTAXIME SODIUM:Antiallergic therapy:0.06), (CEFTRIAXONE SODIUM:Antiallergic therapy:0.6), (CHOLESTYRAMINE:Antiallergic therapy:0.89), (CICLESONIDE:Antiallergic therapy:0.16), (CLOZAPINE:Antiallergic therapy:0.16), (COLCHICINE:Antiallergic therapy:0.58), (CRIZOTINIB:Antiallergic therapy:0.25), (DACLIZUMAB:Antiallergic therapy:0.19), (DASATINIB:Antiallergic therapy:0.95), (DIPYRONE:Antiallergic therapy:0.1), (DOCUSATE CALCIUM:Antiallergic therapy:0.03), (DOFETILIDE:Antiallergic therapy:0.08), (DOXAZOSIN\DOXAZOSIN MESYLATE:Antiallergic therapy:0.33), (DUTASTERIDE:Antiallergic therapy:0.14), (EDOXABAN TOSYLATE:Antiallergic therapy:0.12), (EMPAGLIFLOZIN\LINAGLIPTIN:Antiallergic therapy:0.08), (ENALAPRIL MALEATE:Antiallergic therapy:0.61), (ERYTHROMYCIN:Antiallergic therapy:0.23), (ESTROGENS, CONJUGATED\MEDROXYPROGESTERONE ACETATE:Antiallergic therapy:0.08), (FESOTERODINE FUMARATE:Antiallergic therapy:0.24), (FLECAINIDE:Antiallergic therapy:0.33), (FLUOXETINE\FLUOXETINE HYDROCHLORIDE:Antiallergic therapy:0.38), (GLYCOPYRRONIUM\INDACATEROL:Antiallergic therapy:0.15), (HYDROCHLOROTHIAZIDE\IRBESARTAN:Antiallergic therapy:0.04), (HYDROXYCHLOROQUINE DIPHOSPHATE:Antiallergic therapy:0.03), (HYOSCYAMINE:Antiallergic therapy:0.34), (IMATINIB MESYLATE:Antiallergic therapy:0.69), (IPRATROPIUM:Antiallergic therapy:0.95), (IRINOTECAN:Antiallergic therapy:0.28), (IRINOTECAN HYDROCHLORIDE:Antiallergic therapy:0.61), (ISOSORBIDE:Antiallergic therapy:0.43), (LABETALOL\LABETALOL HYDROCHLORIDE:Antiallergic therapy:0.97), (LACOSAMIDE:Antiallergic therapy:0.14), (LEDIPASVIR\SOFOSBUVIR:Antiallergic therapy:0.16), (LERCANIDIPINE:Antiallergic therapy:0.24), (LEUCOVORIN CALCIUM:Antiallergic therapy:0.71), (LITHIUM CARBONATE:Antiallergic therapy:0.33), (METHENAMINE HIPPURATE:Antiallergic therapy:0.36), (METHYLPREDNISOLONE ACETATE:Antiallergic therapy:0.54), (NABUMETONE:Antiallergic therapy:0.06), (NALOXONE HYDROCHLORIDE\OXYCODONE HYDROCHLORIDE:Antiallergic therapy:0.4), (NEBIVOLOL:Antiallergic therapy:0.65), (NILOTINIB:Antiallergic therapy:1.42), (PANTOPRAZOLE MAGNESIUM:Antiallergic therapy:0.93), (PEGINTERFERON BETA-1A:Antiallergic therapy:0.15), (PEMETREXED:Antiallergic therapy:0.03), (PERINDOPRIL:Antiallergic therapy:0.21), (PHENYTOIN:Antiallergic therapy:0.33), (PHENYTOIN SODIUM:Antiallergic therapy:0), (PONATINIB:Antiallergic therapy:0.14), (PRAVASTATIN:Antiallergic therapy:1.46), (PRIMIDONE:Antiallergic therapy:0.87), (PYRAZINAMIDE:Antiallergic therapy:0.68), (RALOXIFENE HYDROCHLORIDE:Antiallergic therapy:0.06), (RANOLAZINE:Antiallergic therapy:0.71), (ROFLUMILAST:Antiallergic therapy:0.62), (SACUBITRIL\VALSARTAN:Antiallergic therapy:0.71), (SECUKINUMAB:Antiallergic therapy:0.58), (TERIFLUNOMIDE:Antiallergic therapy:0.56), (TETRACYCLINE:Antiallergic therapy:0.04), (THALIDOMIDE:Antiallergic therapy:1.01), (TIOTROPIUM:Antiallergic therapy:0.11), (TOCILIZUMAB:Antiallergic therapy:0.53), (TOFACITINIB CITRATE:Antiallergic therapy:0.25), (TRIAMCINOLONE ACETONIDE:Antiallergic therapy:0.64), (UMECLIDINIUM BROMIDE\VILANTEROL TRIFENATATE:Antiallergic therapy:0.35), (VALGANCICLOVIR HYDROCHLORIDE:Antiallergic therapy:0.22), (VARENICLINE TARTRATE:Antiallergic therapy:0.53), (.ALPHA.1-PROTEINASE INHIBITOR HUMAN:Antibiotic prophylaxis:0.61), (ACETAMINOPHEN\PROPOXYPHENE HYDROCHLORIDE:Antibiotic prophylaxis:0.44), (ACLARUBICIN HYDROCHLORIDE:Antibiotic prophylaxis:0.96), (ADO-TRASTUZUMAB EMTANSINE:Antibiotic prophylaxis:0.68), (ALISKIREN HEMIFUMARATE:Antibiotic prophylaxis:0.12), (ALUMINUM HYDROXIDE\MAGNESIUM OXIDE\OXETHAZAINE:Antibiotic prophylaxis:0.54), (AMINO ACIDS\CARBOHYDRATES\ELECTROLYTES NOS\VITAMINS:Antibiotic prophylaxis:0), (AMISULPRIDE:Antibiotic prophylaxis:0.43), (AMLODIPINE\VALSARTAN:Antibiotic prophylaxis:0.81), (AMMONIUM GLYCYRRHIZATE\CYSTEINE HYDROCHLORIDE\GLYCINE:Antibiotic prophylaxis:0.42), (AMMONIUM LACTATE:Antibiotic prophylaxis:0.77), (AMPICILLIN SODIUM:Antibiotic prophylaxis:0.03), (ANAGRELIDE HYDROCHLORIDE:Antibiotic prophylaxis:0.09), (ANAKINRA:Antibiotic prophylaxis:0.11), (ARGATROBAN:Antibiotic prophylaxis:0.02), (AXITINIB:Antibiotic prophylaxis:0.08), (AZELASTINE:Antibiotic prophylaxis:0.34), (AZELNIDIPINE:Antibiotic prophylaxis:0.16), (AZITHROMYCIN OR METHOTREXATE SODIUM:Antibiotic prophylaxis:0.83), (BALSALAZIDE DISODIUM:Antibiotic prophylaxis:0.33), (BECLOMETHASONE DIPROPIONATE\FORMOTEROL:Antibiotic prophylaxis:0.33), (BENAZEPRIL\BENAZEPRIL HYDROCHLORIDE:Antibiotic prophylaxis:0.82), (BERAPROST:Antibiotic prophylaxis:0.02), (BETAMETHASONE\CALCIPOTRIENE:Antibiotic prophylaxis:0.04), (BIFIDOBACTERIUM LONGUM SUBSP. INFANTIS:Antibiotic prophylaxis:0.03), (BILASTINE:Antibiotic prophylaxis:0.61), (BLESELUMAB:Antibiotic prophylaxis:0.69), (BOTULISM ANTITOXIN:Antibiotic prophylaxis:0.15), (BRINCIDOFOVIR:Antibiotic prophylaxis:0.99), (BUMETANIDE:Antibiotic prophylaxis:0.94), (BUPRENORPHINE HYDROCHLORIDE\NALOXONE HYDROCHLORIDE:Antibiotic prophylaxis:0.18), (CABAZITAXEL:Antibiotic prophylaxis:0.16), (CALCITONIN:Antibiotic prophylaxis:0.68), (CALCITONIN SALMON:Antibiotic prophylaxis:1.08), (CALCIUM CARBONATE\ERGOCALCIFEROL\RETINOL:Antibiotic prophylaxis:0.01), (CALCIUM CARBONATE\VITAMIN D:Antibiotic prophylaxis:0.7), (CALCIUM\MAGNESIUM:Antibiotic prophylaxis:0.25), (CANAGLIFLOZIN\METFORMIN HYDROCHLORIDE:Antibiotic prophylaxis:0.43), (CARBON DIOXIDE:Antibiotic prophylaxis:0.09), (CARVEDILOL PHOSPHATE:Antibiotic prophylaxis:0.96), (CHLORTHALIDONE:Antibiotic prophylaxis:0.98), (CHONDROITIN SULFATE (BOVINE)\GLUCOSAMINE HYDROCHLORIDE:Antibiotic prophylaxis:0.27), (CHONDROITIN SULFATE (CHICKEN):Antibiotic prophylaxis:0.63), (CLOMIPRAMINE HYDROCHLORIDE:Antibiotic prophylaxis:0.44), (COAGULATION FACTOR IX HUMAN\COAGULATION FACTOR VII HUMAN\COAGULATION FACTOR X HUMAN\PROTEIN C\PROTEIN S HUMAN\PROTHROMBIN:Antibiotic prophylaxis:0.02), (COD LIVER OIL:Antibiotic prophylaxis:0.11), (CODEINE PHOSPHATE\GUAIFENESIN:Antibiotic prophylaxis:0.13), (CORTICOTROPIN:Antibiotic prophylaxis:0.04), (CYAMEMAZINE:Antibiotic prophylaxis:0.44), (CYCLOPHOSPHAMIDE\CYTARABINE\DEXAMETHASONE\DOXORU BICIN\METHOTREXATE\VINCRISTINE:Antibiotic prophylaxis:0.57), (DABRAFENIB:Antibiotic prophylaxis:0.42), (DANAZOL:Antibiotic prophylaxis:1.6), (DANTROLENE SODIUM:Antibiotic prophylaxis:0.26), (DARUNAVIR\RITONAVIR:Antibiotic prophylaxis:0.01), (DEFERIPRONE:Antibiotic prophylaxis:0.15), (DEFLAZACORT:Antibiotic prophylaxis:1.06), (DESVENLAFAXINE SUCCINATE:Antibiotic prophylaxis:0.36), (DEXAMETHASONE ACETATE:Antibiotic prophylaxis:1.7), (DIDANOSINE:Antibiotic prophylaxis:0.14), (DIGITOXIN:Antibiotic prophylaxis:0.72), (DIPYRIDAMOLE:Antibiotic prophylaxis:1.31), (DOFETILIDE:Antibiotic prophylaxis:0.62), (DOLASETRON MESYLATE:Antibiotic prophylaxis:0.05), (DORZOLAMIDE HYDROCHLORIDE:Antibiotic prophylaxis:0.34), (DOXEPIN HYDROCHLORIDE:Antibiotic prophylaxis:0.22), (DOXYLAMINE SUCCINATE:Antibiotic prophylaxis:0.32), (DRONEDARONE:Antibiotic prophylaxis:0.29), (DROSPIRENONE\ETHINYL ESTRADIOL:Antibiotic prophylaxis:0.03), (ECONAZOLE:Antibiotic prophylaxis:1.78), (ELTROMBOPAG OLAMINE:Antibiotic prophylaxis:0.13), (EMPAGLIFLOZIN\LINAGLIPTIN:Antibiotic prophylaxis:0.29), (EVOLOCUMAB:Antibiotic prophylaxis:1.02), (FENOFIBRIC ACID:Antibiotic prophylaxis:0.13), (FERROUS SULFATE ANHYDROUS:Antibiotic prophylaxis:0.31), (FERROUS SULFATE\GLYCINE:Antibiotic prophylaxis:0.09), (FESOTERODINE FUMARATE:Antibiotic prophylaxis:1.21), (FLURAZEPAM\FLURAZEPAM HYDROCHLORIDE:Antibiotic prophylaxis:0.05), (FLUTICASONE:Antibiotic prophylaxis:1.02), (FORMOTEROL FUMARATE:Antibiotic prophylaxis:1.21), (FOSFLUCONAZOLE:Antibiotic prophylaxis:0.86), (FOSFOMYCIN:Antibiotic prophylaxis:1.4), (FOSPHENYTOIN SODIUM:Antibiotic prophylaxis:0.55), (FOSPHENYTOIN\FOSPHENYTOIN SODIUM:Antibiotic prophylaxis:1.16), (GABEXATE MESYLATE:Antibiotic prophylaxis:0.13), (GEMCITABINE\GEMCITABINE HYDROCHLORIDE:Antibiotic prophylaxis:0.24), (GIMERACIL\OTERACIL\TEGAFUR:Antibiotic prophylaxis:0), (GLUCAGON:Antibiotic prophylaxis:0.23), (GLYCOPYRROLATE\INDACATEROL MALEATE:Antibiotic prophylaxis:0.23), (GLYCOPYRRONIUM\INDACATEROL:Antibiotic prophylaxis:1.34), (HERBALS\TURMERIC:Antibiotic prophylaxis:0.05), (HUMAN HEPATITIS B VIRUS IMMUNE GLOBULIN:Antibiotic prophylaxis:0), (HYDROCORTISONE ACETATE:Antibiotic prophylaxis:1.2), (HYDROCORTISONE HEMISUCCINATE:Antibiotic prophylaxis:0.12), (HYOSCYAMINE:Antibiotic prophylaxis:0.21), (IBRITUMOMAB TIUXETAN:Antibiotic prophylaxis:1.65), (ILOPROST:Antibiotic prophylaxis:0.39), (IMIPRAMINE HYDROCHLORIDE:Antibiotic prophylaxis:0.86), (INDACATEROL:Antibiotic prophylaxis:0.78), (INFLIXIMAB-DYYB:Antibiotic prophylaxis:0.39), (INSULIN DEGLUDEC:Antibiotic prophylaxis:0.1), (INTERFERON:Antibiotic prophylaxis:0.23), (INTERFERON ALFA-2A:Antibiotic prophylaxis:0.68), (INTERFERON GAMMA-1B:Antibiotic prophylaxis:0.88), (IRINOTECAN HYDROCHLORIDE:Antibiotic prophylaxis:0.25), (IVABRADINE:Antibiotic prophylaxis:0.09), (LACTASE:Antibiotic prophylaxis:0.25), (LANREOTIDE ACETATE:Antibiotic prophylaxis:0.08), (LEVOLEUCOVORIN CALCIUM:Antibiotic prophylaxis:0.16), (LIOTHYRONINE SODIUM:Antibiotic prophylaxis:0.08), (LITHIUM CARBONATE:Antibiotic prophylaxis:1.42), (LOTEPREDNOL ETABONATE:Antibiotic prophylaxis:0.03), (LOVASTATIN:Antibiotic prophylaxis:1.3), (MAGNESIUM ASPARTATE\POTASSIUM ASPARTATE:Antibiotic prophylaxis:0.31), (MECLIZINE\MECLIZINE HYDROCHLORIDE:Antibiotic prophylaxis:0.41), (MELPERONE:Antibiotic prophylaxis:0.26), (MELPHALAN HYDROCHLORIDE:Antibiotic prophylaxis:0.19), (MENATETRENONE:Antibiotic prophylaxis:0.13), (MEPOLIZUMAB:Antibiotic prophylaxis:0.46), (METFORMIN HYDROCHLORIDE\VILDAGLIPTIN:Antibiotic prophylaxis:0.35), (METHENOLONE ENANTHATE:Antibiotic prophylaxis:1.22), (METHOTREXATE\METHOTREXATE SODIUM:Antibiotic prophylaxis:0.27), (METHYLPREDNISOLONE ACETATE:Antibiotic prophylaxis:1.53), (METHYLPREDNISOLONE OR METHYLPREDNISOLONE ACETATE:Antibiotic prophylaxis:0.1), (MIDODRINE HYDROCHLORIDE:Antibiotic prophylaxis:0.2), (MOGAMULIZUMAB:Antibiotic prophylaxis:0.59), (MORPHINE SULFATE\NALTREXONE HYDROCHLORIDE:Antibiotic prophylaxis:0.23), (NABUMETONE:Antibiotic prophylaxis:0.58), (NALBUPHINE HYDROCHLORIDE:Antibiotic prophylaxis:0.21), (NALOXONE:Antibiotic prophylaxis:0.82), (NATEGLINIDE:Antibiotic prophylaxis:0.43), (OCTREOTIDE ACETATE:Antibiotic prophylaxis:0.08), (OLMESARTAN:Antibiotic prophylaxis:0.62), (OLODATEROL HYDROCHLORIDE\TIOTROPIUM BROMIDE MONOHYDRATE:Antibiotic prophylaxis:0.8), (ORPHENADRINE:Antibiotic prophylaxis:0.04), (OXCARBAZEPINE:Antibiotic prophylaxis:2.22), (PANCRELIPASE:Antibiotic prophylaxis:0.53), (PANTOPRAZOLE MAGNESIUM:Antibiotic prophylaxis:1.68), (PARICALCITOL:Antibiotic prophylaxis:0.51), (PAZUFLOXACIN:Antibiotic prophylaxis:0.57), (PAZUFLOXACIN MESILATE:Antibiotic prophylaxis:0.01), (PEGINTERFERON ALFA-2B:Antibiotic prophylaxis:0.09), (PEGINTERFERON BETA-1A:Antibiotic prophylaxis:0.54), (PEMETREXED:Antibiotic prophylaxis:0.61), (PENTOBARBITAL:Antibiotic prophylaxis:0.14), (PHENOBARBITAL SODIUM:Antibiotic prophylaxis:0.39), (PHENPROCOUMON:Antibiotic prophylaxis:0.91), (PILSICAINIDE HYDROCHLORIDE:Antibiotic prophylaxis:0.27), (PIMOBENDAN:Antibiotic prophylaxis:0.38), (PIOGLITAZONE:Antibiotic prophylaxis:0.17), (PIPERACILLIN SODIUM:Antibiotic prophylaxis:0.22), (PIRARUBICIN:Antibiotic prophylaxis:0.04), (PIRITRAMIDE:Antibiotic prophylaxis:0.5), (PIROXICAM:Antibiotic prophylaxis:0.37), (PNEUMOCOCCAL VACCINE, POLYVALENT 23:Antibiotic prophylaxis:0.44), (POLYMYXIN B SULFATE\TRIMETHOPRIM SULFATE:Antibiotic prophylaxis:0.22), (POTASSIUM CITRATE\TRISODIUM CITRATE DIHYDRATE:Antibiotic prophylaxis:0.42), (PRANLUKAST:Antibiotic prophylaxis:0.26), (PRAZOSIN HYDROCHLORIDE:Antibiotic prophylaxis:0.13), (PREDNISOLONE SODIUM SUCCINATE:Antibiotic prophylaxis:0.55), (PROCARBAZINE:Antibiotic prophylaxis:0.06), (PSYLLIUM HUSK:Antibiotic prophylaxis:0.34), (PYRIMETHAMINE\SULFADOXINE:Antibiotic prophylaxis:0.02), (QUINACRINE:Antibiotic prophylaxis:0.07), (QUINAPRIL:Antibiotic prophylaxis:0.1), (RALOXIFENE HYDROCHLORIDE:Antibiotic prophylaxis:0.11), (RANOLAZINE:Antibiotic prophylaxis:1.08), (REGORAFENIB:Antibiotic prophylaxis:0.12), (RIVASTIGMINE TARTRATE:Antibiotic prophylaxis:0.8), (RIZATRIPTAN BENZOATE:Antibiotic prophylaxis:0.67), (SACUBITRIL\VALSARTAN:Antibiotic prophylaxis:0.6), (SARPOGRELATE:Antibiotic prophylaxis:0.73), (SCOPOLAMINE HYDROBROMIDE:Antibiotic prophylaxis:0.08), (SIPULEUCEL-T:Antibiotic prophylaxis:0.04), (SODIUM CITRATE:Antibiotic prophylaxis:0.78), (SODIUM PHOSPHATE, DIBASIC\SODIUM PHOSPHATE, MONOBASIC:Antibiotic prophylaxis:0.69), (SUGAMMADEX SODIUM:Antibiotic prophylaxis:0.75), (TAFLUPROST:Antibiotic prophylaxis:0.06), (TAMOXIFEN CITRATE:Antibiotic prophylaxis:0.05), (TEDUGLUTIDE\WATER:Antibiotic prophylaxis:0.1), (TENIPOSIDE:Antibiotic prophylaxis:0.05), (TESTOSTERONE UNDECANOATE:Antibiotic prophylaxis:0.26), (TETRACAINE HYDROCHLORIDE:Antibiotic prophylaxis:0.21), (TETRACYCLINE:Antibiotic prophylaxis:0.97), (THEOPHYLLINE:Antibiotic prophylaxis:0.13), (THIAMYLAL SODIUM:Antibiotic prophylaxis:1.28), (TICLOPIDINE:Antibiotic prophylaxis:0.09), (TRAMETINIB:Antibiotic prophylaxis:0.24), (TRANDOLAPRIL:Antibiotic prophylaxis:0.27), (TRIAMTERENE:Antibiotic prophylaxis:0.26), (TRIMIPRAMINE MALEATE:Antibiotic prophylaxis:0.04), (TROPATEPINE:Antibiotic prophylaxis:0.44), (UMECLIDINIUM BROMIDE:Antibiotic prophylaxis:0.26), (UMECLIDINIUM BROMIDE\VILANTEROL TRIFENATATE:Antibiotic prophylaxis:1.03), (VINDESINE SULFATE:Antibiotic prophylaxis:0.41), (VINORELBINE TARTRATE:Antibiotic prophylaxis:0.25), (VINORELBINE\VINORELBINE TARTRATE:Antibiotic prophylaxis:0.31), (VITAMIN A:Antibiotic prophylaxis:0.84), (XIPAMIDE:Antibiotic prophylaxis:0.13), (.ALPHA.1-PROTEINASE INHIBITOR HUMAN:Antibiotic therapy:1.03), (ACARBOSE:Antibiotic therapy:0.79), (ACETAMINOPHEN\CAFFEINE\OPIUM:Antibiotic therapy:0.12), (ACETAMINOPHEN\PROPOXYPHENE NAPSYLATE:Antibiotic therapy:0.28), (ACLARUBICIN HYDROCHLORIDE:Antibiotic therapy:0.2), (AGALSIDASE BETA:Antibiotic therapy:0.62), (ALCOHOL:Antibiotic therapy:0.7), (ALGLUCOSIDASE ALFA:Antibiotic therapy:0.22), (ALISKIREN:Antibiotic therapy:0.29), (ALUMINUM HYDROXIDE\MAGNESIUM OXIDE\OXETHAZAINE:Antibiotic therapy:0.31), (AMILORIDE HYDROCHLORIDE\HYDROCHLOROTHIAZIDE:Antibiotic therapy:0.35), (AMISULPRIDE:Antibiotic therapy:0.62), (AMLODIPINE BESYLATE\ATORVASTATIN CALCIUM:Antibiotic therapy:0.01), (AMLODIPINE BESYLATE\HYDROCHLOROTHIAZIDE\OLMESARTAN MEDOXOMIL:Antibiotic therapy:0.09), (AMLODIPINE\VALSARTAN:Antibiotic therapy:0.48), (AMMONIUM LACTATE:Antibiotic therapy:0.22), (ANASTROZOLE:Antibiotic therapy:1.95),(ARMODAFINIL:Antibiotic therapy:1.11), (ARSENIC TRIOXIDE:Antibiotic therapy:0.06), (ASPARAGINASE ERWINIA CHRYSANTHEMI:Antibiotic therapy:0.27), (ASPIRIN\MAGNESIUM OXIDE:Antibiotic therapy:0.13), (ATAZANAVIR:Antibiotic therapy:0.05), (ATROPINE SULFATE:Antibiotic therapy:0.22), (AXITINIB:Antibiotic therapy:0.07), (AZELASTINE:Antibiotic therapy:0.35), (BELATACEPT:Antibiotic therapy:0.84), (BIFIDOBACTERIUM LONGUM SUBSP. INFANTIS:Antibiotic therapy:0.04), (BLESELUMAB:Antibiotic therapy:0.14), (BLINATUMOMAB:Antibiotic therapy:2.21), (BOTULINUM TOXIN TYPE A:Antibiotic therapy:0.05), (BROTIZOLAM:Antibiotic therapy:0.41), (BUPRENORPHINE HYDROCHLORIDE\NALOXONE HYDROCHLORIDE:Antibiotic therapy:0.26), (CALCIUM CARBONATE\VITAMIN D:Antibiotic therapy:0.93), (CALCIUM PHOSPHATE, DIBASIC, ANHYDROUS\FERROUS SUCCINATE\MAGNESIUM HYDROXIDE:Antibiotic therapy:0.05), (CANDESARTAN CILEXETIL\\HYDROCHLOROTHIAZIDE:Antibiotic therapy:1.87), (CAPSAICIN:Antibiotic therapy:0.01), (CARBIMAZOLE:Antibiotic therapy:0.35), (CARMUSTINE:Antibiotic therapy:0.03), (CASANTHRANOL\DOCUSATE SODIUM:Antibiotic therapy:0.21), (CEFOZOPRAN:Antibiotic therapy:0.03), (CEFPROZIL:Antibiotic therapy:0.06), (CHLORPHENIRAMINE\HYDROCODONE:Antibiotic therapy:0.18), (CHONDROITIN SULFATE (BOVINE)\GLUCOSAMINE HYDROCHLORIDE:Antibiotic therapy:0.32), (CINACALCET HYDROCHLORIDE:Antibiotic therapy:0.59), (CLOFAZIMINE:Antibiotic therapy:0.14), (CLOMIPRAMINE HYDROCHLORIDE:Antibiotic therapy:0.28), (COD LIVER OIL:Antibiotic therapy:0.06), (CORTICOTROPIN:Antibiotic therapy:0.24), (CORTISONE\HYDROCORTISONE:Antibiotic therapy:1.15), (CYCLOPHOSPHAMIDE\CYTARABINE\DEXAMETHASONE\DOXORUBICI N\METHOTREXATE\VINCRISTINE:Antibiotic therapy:0.32), (CYPROHEPTADINE HYDROCHLORIDE:Antibiotic therapy:0.14), (DABRAFENIB:Antibiotic therapy:0.6), (DACLIZUMAB:Antibiotic therapy:1.57), (DANAZOL:Antibiotic therapy:0.98), (DESONIDE:Antibiotic therapy:0.27), (DIMETHICONE\SILICON DIOXIDE\SIMETHICONE:Antibiotic therapy:0.32), (DOLASETRON MESYLATE:Antibiotic therapy:0.18), (DOXEPIN HYDROCHLORIDE:Antibiotic therapy:0.15), (DOXYLAMINE SUCCINATE:Antibiotic therapy:0.15), (DRONEDARONE:Antibiotic therapy:0.3), (DUTASTERIDE\TAMSULOSIN HYDROCHLORIDE:Antibiotic therapy:0.04), (EDOXABAN TOSYLATE:Antibiotic therapy:1.17), (ELETRIPTAN HYDROBROMIDE:Antibiotic therapy:1.18), (ELOTUZUMAB:Antibiotic therapy:1), (ELTROMBOPAG OLAMINE:Antibiotic therapy:0.03), (EMPAGLIFLOZIN\LINAGLIPTIN:Antibiotic therapy:0.68), (EPHEDRINE:Antibiotic therapy:0.12), (EVOLOCUMAB:Antibiotic therapy:1.06), (FENOFIBRIC ACID:Antibiotic therapy:0.23), (FENOTEROL HYDROBROMIDE:Antibiotic therapy:0.38), (FERROUS SULFATE ANHYDROUS:Antibiotic therapy:0.13), (FESOTERODINE FUMARATE:Antibiotic therapy:0.97), (FLUTICASONE FUROATE:Antibiotic therapy:0.8), (FLUTICASONE FUROATE\VILANTEROL TRIFENATATE:Antibiotic therapy:1.27), (FLUVOXAMINE MALEATE:Antibiotic therapy:0.14), (FORMOTEROL FUMARATE\MOMETASONE FUROATE:Antibiotic therapy:0.89), (FOSPHENYTOIN\FOSPHENYTOIN SODIUM:Antibiotic therapy:0.94), (GIMERACIL\OTERACIL\TEGAFUR:Antibiotic therapy:0.31), (GLYCOPYRRONIUM\INDACATEROL:Antibiotic therapy:1.19), (GOSERELIN ACETATE:Antibiotic therapy:0.23), (GUANFACINE HYDROCHLORIDE:Antibiotic therapy:0.05), (GUSPERIMUS TRIHYDROCHLORIDE:Antibiotic therapy:0.25), (HERBALS\TURMERIC:Antibiotic therapy:0.19), (HYDROCHLOROTHIAZIDE\TELMISARTAN:Antibiotic therapy:0.71), (HYDROCORTISONE ACETATE:Antibiotic therapy:0.77), (HYDROXYCHLOROQUINE DIPHOSPHATE:Antibiotic therapy:0.35), (IBRITUMOMAB TIUXETAN:Antibiotic therapy:1.3), (INDACATEROL:Antibiotic therapy:0.41), (INFLIXIMAB-DYYB:Antibiotic therapy:0.19), (INTERFERON ALFA-2A:Antibiotic therapy:0.24), (INTERFERON ALFA-2B:Antibiotic therapy:0.19), (INTERFERON GAMMA-1B:Antibiotic therapy:0.87), (IXAZOMIB:Antibiotic therapy:0.44), (LAPATINIB DITOSYLATE:Antibiotic therapy:0.2), (LIDOCAINE\PRILOCAINE:Antibiotic therapy:1.03), (LIOTHYRONINE:Antibiotic therapy:0.01), (LIRAGLUTIDE:Antibiotic therapy:0.1), (MAGNESIUM ASPARTATE HYDROCHLORIDE:Antibiotic therapy:0.27), (MAGNESIUM ASPARTATE\POTASSIUM ASPARTATE:Antibiotic therapy:0.03), (MEPERIDINE:Antibiotic therapy:0.89), (METFORMIN HYDROCHLORIDE\VILDAGLIPTIN:Antibiotic therapy:0.34), (METHENAMINE HIPPURATE:Antibiotic therapy:0.83), (METHENOLONE ENANTHATE:Antibiotic therapy:0.04), (MODAFINIL:Antibiotic therapy:0.48), (NIRAPARIB:Antibiotic therapy:0.19), (NORETHINDRONE:Antibiotic therapy:0.56), (OLODATEROL HYDROCHLORIDE\TIOTROPIUM BROMIDE MONOHYDRATE:Antibiotic therapy:0.53), (OLOPATADINE:Antibiotic therapy:0.74), (PANITUMUMAB:Antibiotic therapy:0.29), (PATIROMER:Antibiotic therapy:0.06), (PEGINTERFERON BETA-1A: Antibiotic therapy:0.44), (PEMETREXED:Antibiotic therapy:0.62), (PENTOSTATIN:Antibiotic therapy:1.14), (PENTOXIFYLLINE:Antibiotic therapy:0.39), (PIMECROLIMUS:Antibiotic therapy:0.1), (PIRITRAMIDE:Antibiotic therapy:0.06), (PIROXICAM:Antibiotic therapy:0.35), (PNEUMOCOCCAL VACCINE, POLYVALENT 23:Antibiotic therapy:0.13), (PRAZOSIN HYDROCHLORIDE:Antibiotic therapy:0.1), (PREDNISOLONE SODIUM PHOSPHATE:Antibiotic therapy:1.21), (PROPAFENONE:Antibiotic therapy:0.09), (PSYLLIUM HUSK:Antibiotic therapy:0.17), (QUINAPRIL:Antibiotic therapy:0.04), (RADIATION THERAPY:Antibiotic therapy:0.44), (RALOXIFENE HYDROCHLORIDE:Antibiotic therapy:0.17), (RAMUCIRUMAB:Antibiotic therapy:0.09), (REGORAFENIB:Antibiotic therapy:0.51), (RETINOL:Antibiotic therapy:0.53), (RIOCIGUAT:Antibiotic therapy:0.03), (RIVASTIGMINE TARTRATE:Antibiotic therapy:0.42), (RIZATRIPTAN:Antibiotic therapy:0.02), (RIZATRIPTAN BENZOATE:Antibiotic therapy:0.78), (ROPINIROLE HYDROCHLORIDE:Antibiotic therapy:0.73), (ROPIVACAINE:Antibiotic therapy:0.64), (SODIUM CITRATE:Antibiotic therapy:0.41), (SODIUM OXYBATE:Antibiotic therapy:0.92), (SODIUM PHOSPHATE, DIBASIC\SODIUM PHOSPHATE, MONOBASIC:Antibiotic therapy:0.31), (TERAZOSIN HYDROCHLORIDE:Antibiotic therapy:0.31), (TERBINAFINE HYDROCHLORIDE:Antibiotic therapy:0.74), (THEOPHYLLINE:Antibiotic therapy:0.06), (THIAMYLAL SODIUM:Antibiotic therapy:0.12), (THIOPENTAL:Antibiotic therapy:0.7), (TIMOLOL:Antibiotic therapy:0.94), (TINZAPARIN:Antibiotic therapy:0.89), (TISAGENLECLEUCEL:Antibiotic therapy:0.14), (TRAMETINIB:Antibiotic therapy:0.62), (TRANDOLAPRIL:Antibiotic therapy:0.04), (TRIAMTERENE:Antibiotic therapy:0.27), (UMECLIDINIUM BROMIDE:Antibiotic therapy:0.21), (UMECLIDINIUM BROMIDE\VILANTEROL TRIFENATATE:Antibiotic therapy:0.98), (VILAZODONE HYDROCHLORIDE:Antibiotic therapy:0.13), (VINBLASTINE:Antibiotic therapy:0), (VINORELBINE TARTRATE:Antibiotic therapy:0.46), (ZIV-AFLIBERCEPT:Antibiotic therapy:0.11), (.ALPHA.-TOCOPHEROL\VITAMINS:Anticoagulant therapy:0.09), (.BETA.-HYDROXYISOVALERIC ACID\ARGININE\GLUTAMINE:Anticoagulant therapy:0.18), (ABACAVIR SULFATE\DOLUTEGRAVIR SODIUM\LAMIVUDINE:Anticoagulant therapy:0.31), (ABOBOTULINUMTOXINA:Anticoagulant therapy:0.84), (ACECLOFENAC:Anticoagulant therapy:0.26), (ACETAMINOPHEN\METHOCARBAMOL:Anticoagulant therapy:0.19), (ACETIC ACID\SODIUM ACETATE:Anticoagulant therapy:0.31), (ACTIVATED CHARCOAL:Anticoagulant therapy:1.18), (ADO-TRASTUZUMAB EMTANSINE:Anticoagulant therapy:2.18), (ALBUTEROL\IPRATROPIUM BROMIDE:Anticoagulant therapy:0.28), (ALDESLEUKIN:Anticoagulant therapy:0.79), (ALISKIREN HEMIFUMARATE\HYDROCHLOROTHIAZIDE:Anticoagulant therapy:0.35), (ALMOTRIPTAN MALATE:Anticoagulant therapy:0.05), (AMANTADINE:Anticoagulant therapy:2.22), (AMILORIDE HYDROCHLORIDE\FUROSEMIDE:Anticoagulant therapy:0.19), (AMINO ACIDS\CALCIUM CHLORIDE\MAGNESIUM CHLORIDE\SODIUM CHLORIDE\SODIUM LACTATE:Anticoagulant therapy:0), (AMINO ACIDS\DEXTROSE\ELECTROLYTES NOS:Anticoagulant therapy:1.87), (AMISULPRIDE:Anticoagulant therapy:1.52), (AMLODIPINE BESYLATE\IRBESARTAN:Anticoagulant therapy:0.18), (AMMONIUM CHLORIDE\DEXTROMETHORPHAN\SODIUM CITRATE:Anticoagulant therapy:0.79), (AMOXICILLIN\CLARITHROMYCIN\ESOMEPRAZOLE:Anticoagulant therapy:0.93), (ANAKINRA:Anticoagulant therapy:0.5), (ANIDULAFUNGIN:Anticoagulant therapy:0.12), (ANTIHEMOPHILIC FACTOR, HUMAN RECOMBINANT:Anticoagulant therapy:0.28), (ANTIHISTAMINES NOS:Anticoagulant therapy:1.22), (ARIPIPRAZOLE LAUROXIL:Anticoagulant therapy:0.68), (ASCORBIC ACID/CHROMIUM/SELENIUM/VANADYL SULFATE:Anticoagulant therapy:0.44), (ASPERGILLUS NIGER ALPHA.-GALACTOSIDASE:Anticoagulant therapy:0.27), (ASPIRIN\CAFFEINE\SALICYLAMIDE:Anticoagulant therapy:0.37), (ASPIRIN\MAGNESIUM HYDROXIDE:Anticoagulant therapy:1.02), (ASUNAPREVIR:Anticoagulant therapy:0.69), (ATRACURIUM BESYLATE:Anticoagulant therapy:0.71), (AZITHROMYCIN OR METHOTREXATE SODIUM:Anticoagulant therapy:0.05), (BENIDIPINE:Anticoagulant therapy:0.45), (BENIDIPINE HYDROCHLORIDE:Anticoagulant therapy:0.26), (BEPOTASTINE BESILATE:Anticoagulant therapy:0.27), (BETAHISTINE MESILATE:Anticoagulant therapy:0.25), (BIFIDOBACTERIUM LONGUM\LACTOBACILLUS ACIDOPHILUS\LACTOBACILLUS RHAMNOSUS:Anticoagulant therapy:0.05), (BIFIDOBACTERIUM SPP.:Anticoagulant therapy:0.4), (BISACODYL\GLYCERIN\MINERAL OIL\SODIUM PHOSPHATE, DIBASIC\SODIUM PHOSPHATE, MONOBASIC:Anticoagulant therapy:0.22), (BLESELUMAB:Anticoagulant therapy:0.05), (BLINATUMOMAB:Anticoagulant therapy:1.21), (BOTULINUM TOXIN TYPE A:Anticoagulant therapy:0.94), (BROMFENAC SODIUM:Anticoagulant therapy:0.34), (BUPIVACAINE HYDROCHLORIDE:Anticoagulant therapy:1.13), (BUPIVACAINE\EPINEPHRINE:Anticoagulant therapy:0.22), (BUPRENORPHINE\NALOXONE:Anticoagulant therapy:0.19), (CABAZITAXEL:Anticoagulant therapy:0.67), (CALCIUM BICARBONATE:Anticoagulant therapy:0.01), (CALCIUM CARBONATE\MAGNESIUM CARBONATE\MAGNESIUM TRISILICATE:Anticoagulant therapy:0.18), (CALCIUM PHOSPHATE:Anticoagulant therapy:0.12), (CALCIUM\MAGNESIUM:Anticoagulant therapy:1.86), (CARBAMIDE PEROXIDE:Anticoagulant therapy:0.11), (CARBON DIOXIDE:Anticoagulant therapy:0.07), (CARBOXYMETHYLCELLULOSE SODIUM\GLYCERIN\POLYSORBATE 80:Anticoagulant therapy:0.4), (CARTEOLOL:Anticoagulant therapy:0.26), (CARTEOLOL HYDROCHLORIDE:Anticoagulant therapy:0.61), (CEFEPIME\CEFEPIME HYDROCHLORIDE:Anticoagulant therapy:1.36), (CEFOTAXIME\CEFOTAXIME SODIUM:Anticoagulant therapy:0.67), (CEFTAZIDIME SODIUM:Anticoagulant therapy:0.31), (CEFUROXIME SODIUM:Anticoagulant therapy:0.15), (CERTOPARIN SODIUM:Anticoagulant therapy:0.85), (CHONDROITIN SULFATE (CHICKEN):Anticoagulant therapy:0.6), (CICLOPIROX OLAMINE:Anticoagulant therapy:0.3), (CIFENLINE:Anticoagulant therapy:0.13), (CILNIDIPINE:Anticoagulant therapy:0.52), (CIPROFLOXACIN\DEXAMETHASONE:Anticoagulant therapy:0.16), (CISAPRIDE:Anticoagulant therapy:0.22), (CITICOLINE:Anticoagulant therapy:0.05), (CLENBUTEROL HYDROCHLORIDE:Anticoagulant therapy:0.15), (COBICISTAT\ELVITEGRAVIR\EMTRICITABINE\TENOFOVIR ALAFENAMIDE FUMARATE:Anticoagulant therapy:0.31), (COBICISTAT\ELVITEGRAVIR\EMTRICITABINE\TENOFOVIR DISOPROXIL FUMARATE:Anticoagulant therapy:0.01), (CODEINE PHOSPHATE\PROMETHAZINE:Anticoagulant therapy:0.11), (COLISTIN:Anticoagulant therapy:0.54), (CONVALLARIA MAJALIS\CRATAEGUS LAEVIGATA LEAF\PROXYPHYLLINE:Anticoagulant therapy:0.35), (CRANBERRY JUICE:Anticoagulant therapy:0.32), (CROTALIDAE POLYVALENT IMMUNE FAB (OVINE):Anticoagulant therapy:0.51), (CYAMEMAZINE:Anticoagulant therapy:0.32), (CYANOCOBALAMIN\FOLIC ACID\PYRIDOXINE:Anticoagulant therapy:0.64), (CYPROHEPTADINE:Anticoagulant therapy:1.81), (DANTROLENE SODIUM:Anticoagulant therapy:1.1), (DASATINIB:Anticoagulant therapy:3.39), (DECITABINE:Anticoagulant therapy:0.17), (DESIPRAMINE:Anticoagulant therapy:0.02), (DEXTROSE\HEPARIN:Anticoagulant therapy:0.98), (DIMETHICONE 410:Anticoagulant therapy:0.19), (DIMETHINDENE:Anticoagulant therapy:0.48), (DIMETHYL SULFOXIDE:Anticoagulant therapy:0.32), (DIPHENHYDRAMINE HYDROCHLORIDE\LIDOCAINE HYDROCHLORIDE\NYSTATIN:Anticoagulant therapy:0.51), (DOLASETRON MESYLATE:Anticoagulant therapy:1.3), (EFINACONAZOLE:Anticoagulant therapy:0.58), (ELTROMBOPAG OLAMINE:Anticoagulant therapy:1.31), (EMPAGLIFLOZIN\METFORMIN HYDROCHLORIDE:Anticoagulant therapy:0.11), (EMTRICITABINE\RILPIVIRINE HYDROCHLORIDE\TENOFOVIR DISOPROXIL FUMARATE:Anticoagulant therapy:0.01), (EPHEDRINE HYDROCHLORIDE\GUAIFENESIN:Anticoagulant therapy:0.19), (ERIBULIN:Anticoagulant therapy:0.88), (ESTRADIOL\NORETHINDRONE ACETATE:Anticoagulant therapy:0.78), (ESTROPIPATE:Anticoagulant therapy:0.05), (ETHINYL ESTRADIOL\GESTODENE:Anticoagulant therapy:0.34), (ETHINYL ESTRADIOL\NORGESTIMATE:Anticoagulant therapy:0.75), (ETHINYL ESTRADIOL\NORGESTREL:Anticoagulant therapy:0.59), (ETIMICIN SULFATE:Anticoagulant therapy:0.13), (ETOPOSIDE PHOSPHATE:Anticoagulant therapy:0.39), (ETRETINATE:Anticoagulant therapy:0.51), (FENOTEROL HYDROBROMIDE\IPRATROPIUM BROMIDE:Anticoagulant therapy:1.73), (FERRIC HYDROXIDE:Anticoagulant therapy:1.11), (FERRIC POLYSACCHARIDE COMPLEX:Anticoagulant therapy:0.25), (FERROUS SULFATE\GLYCINE:Anticoagulant therapy:1.1), (FIBRINOGEN HUMAN:Anticoagulant therapy:1.68), (FLUPHENAZINE:Anticoagulant therapy:0.07), (FLURAZEPAM HYDROCHLORIDE:Anticoagulant therapy:0.01), (FLURBIPROFEN:Anticoagulant therapy:0.44), (FLUVOXAMINE:Anticoagulant therapy:0.39), (FORMOTEROL FUMARATE\MOMETASONE FUROATE:Anticoagulant therapy:1.54), (FOSPHENYTOIN\FOSPHENYTOIN SODIUM:Anticoagulant therapy:0.71), (FUROSEMIDE\SPIRONOLACTONE:Anticoagulant therapy:0.53), (GLIMEPIRIDE\ROSIGLITAZONE MALEATE:Anticoagulant therapy:1.26), (GOLD:Anticoagulant therapy:0.08), (GUANFACINE HYDROCHLORIDE:Anticoagulant therapy:0.56), (HALOPERIDOL DECANOATE:Anticoagulant therapy:0.19), (HYDRAZINE SULFATE:Anticoagulant therapy:0.34), (HYDROCODONE BITARTRATE\IBUPROFEN:Anticoagulant therapy:1.18), (HYDROXYPROPYL CELLULOSE:Anticoagulant therapy:0.05), (HYOSCYAMINE SULFATE\METHENAMINE\METHYLENE BLUE\PHENYL SALICYLATE\SODIUM PHOSPHATE, MONOBASIC, MONOHYDRATE:Anticoagulant therapy:0.39), (IBRITUMOMAB TIUXETAN:Anticoagulant therapy:0.05), (INDACATEROL MALEATE:Anticoagulant therapy:0.03), (INFLIXIMAB-DYYB:Anticoagulant therapy:0.53), (INSULIN ASPART\INSULIN DEGLUDEC:Anticoagulant therapy:0.07), (INSULIN PORK\INSULIN PURIFIED PORK:Anticoagulant therapy:0.66), (INTERFERON:Anticoagulant therapy:2.19), (INTERFERON ALFA-2A:Anticoagulant therapy:1.95), (INTERFERON ALFA-2B:Anticoagulant therapy:1.74), (INTERFERON GAMMA-1B:Anticoagulant therapy:0.43), (IRON\POLYSACCHARIDES:Anticoagulant therapy:0.67), (IRON\VITAMINS:Anticoagulant therapy:0.38), (IVERMECTIN:Anticoagulant therapy:0.34), (KETAMINE HYDROCHLORIDE:Anticoagulant therapy:1.05), (LAFUTIDINE:Anticoagulant therapy:0.4), (LAPATINIB:Anticoagulant therapy:0.1), (LARONIDASE:Anticoagulant therapy:0.09), (LATANOPROST\TIMOLOL:Anticoagulant therapy:0.42), (LENVATINIB:Anticoagulant therapy:1.07), (LEVOBUPIVACAINE:Anticoagulant therapy:0.26), (LEVOTHYROXINE SODIUM\POTASSIUM IODIDE:Anticoagulant therapy:0.99), (LIMAPROST:Anticoagulant therapy:0.31), (LINSIDOMINE:Anticoagulant therapy:0.84), (LUTETIUM LU-177:Anticoagulant therapy:0.77), (MAGNESIUM ASPARTATE HYDROCHLORIDE:Anticoagulant therapy:1.08), (MAGNESIUM ASPARTATE\POTASSIUM ASPARTATE:Anticoagulant therapy:0.61), (MAGNESIUM GLUCONATE:Anticoagulant therapy:0.75), (MANIDIPINE:Anticoagulant therapy:0.19), (MELPERONE:Anticoagulant therapy:0.65), (METFORMIN HYDROCHLORIDE\ROSIGLITAZONE MALEATE:Anticoagulant therapy:1.61), (METFORMIN HYDROCHLORIDE\SAXAGLIPTIN:Anticoagulant therapy:0.54), (METHENOLONE ENANTHATE:Anticoagulant therapy:1.12), (METHYLENE BLUE:Anticoagulant therapy:0.01), (METHYLNALTREXONE BROMIDE:Anticoagulant therapy:0.1), (METRELEPTIN:Anticoagulant therapy:1.03), (MIGLUSTAT:Anticoagulant therapy:0.31), (MINERAL OIL\PETROLATUM:Anticoagulant therapy:0.31), (MITOXANTRONE HYDROCHLORIDE:Anticoagulant therapy:0.04), (MIZORIBINE:Anticoagulant therapy:0.72), (MOLSIDOMINE:Anticoagulant therapy:0.67), (NALOXONE:Anticoagulant therapy:1.31), (NAPROXEN SODIUM\SUMATRIPTAN SUCCINATE:Anticoagulant therapy:0.27), (NARATRIPTAN HYDROCHLORIDE:Anticoagulant therapy:1.03), (NEFAZODONE:Anticoagulant therapy:0.26), (NEFAZODONE HYDROCHLORIDE:Anticoagulant therapy:0.34), (NEOMYCIN:Anticoagulant therapy:0.61), (ORPHENADRINE:Anticoagulant therapy:1.26), (PARATHYROID HORMONE:Anticoagulant therapy:0.8), (PENICILLAMINE:Anticoagulant therapy:0.04), (PENTOSTATIN:Anticoagulant therapy:0.83), (PERPHENAZINE:Anticoagulant therapy:0.5), (PHOSPHORUS:Anticoagulant therapy:0.15), (PIMOBENDAN:Anticoagulant therapy:0.88), (PINDOLOL:Anticoagulant therapy:0.5), (PITOFENONE:Anticoagulant therapy:0.06), (POLYMYXIN B SULFATE\TRIMETHOPRIM SULFATE:Anticoagulant therapy:0.44), (PORFIMER SODIUM:Anticoagulant therapy:0.35), (POTASSIUM IODIDE:Anticoagulant therapy:0.02), (POTASSIUM PHOSPHATE, MONOBASIC:Anticoagulant therapy:0.64), (PRAMOXINE HYDROCHLORIDE:Anticoagulant therapy:0.24), (PRAZEPAM:Anticoagulant therapy:0.69), (PREDNISOLONE SODIUM PHOSPHATE:Anticoagulant therapy:0.85), (PREGNENOLONE:Anticoagulant therapy:0.15), (PROBUCOL:Anticoagulant therapy:0.26), (PROCATEROL HYDROCHLORIDE:Anticoagulant therapy:0.02), (PRUCALOPRIDE SUCCINATE:Anticoagulant therapy:0.31), (QUINACRINE:Anticoagulant therapy:0.8), (RASBURICASE:Anticoagulant therapy:0.42), (RETEPLASE:Anticoagulant therapy:0.91), (ROPIVACAINE HYDROCHLORIDE:Anticoagulant therapy:0.92), (SARGRAMOSTIM:Anticoagulant therapy:0.06), (SIBUTRAMINE HYDROCHLORIDE:Anticoagulant therapy:0.64), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Anticoagulant therapy:0.02), (SOFOSBUVIR\VELPATASVIR:Anticoagulant therapy:0.9), (SORBITOL:Anticoagulant therapy:0.95), (SUPLATAST TOSILATE:Anticoagulant therapy:0.11), (TEDUGLUTIDE:Anticoagulant therapy:0.55), (TEMSIROLIMUS:Anticoagulant therapy:0.65), (TENECTEPLASE:Anticoagulant therapy:1.76), (TENELIGLIPTIN:Anticoagulant therapy:0.07), (TESAMORELIN:Anticoagulant therapy:0.21), (TETRAZEPAM:Anticoagulant therapy:0.04), (THEOPHYLLINE:Anticoagulant therapy:0.83), (THIAMYLAL SODIUM:Anticoagulant therapy:0.27), (TIBOLONE:Anticoagulant therapy:0.58), (TILIDINE:Anticoagulant therapy:0.55), (TREPROSTINIL SODIUM:Anticoagulant therapy:0.43), (TRIHEXYPHENIDYL:Anticoagulant therapy:0.43), (TRIHEXYPHENIDYL HYDROCHLORIDE:Anticoagulant therapy:0.07), (VANDETANIB:Anticoagulant therapy:0.03), (VINORELBINE\VINORELBINE TARTRATE:Anticoagulant therapy:0.67), (ZICONOTIDE ACETATE:Anticoagulant therapy:0.92), (ZILEUTON:Anticoagulant therapy:1.24), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Antidepressant therapy:0.1), (.ALPHA.1-PROTEINASE INHIBITOR HUMAN:Antidepressant therapy:0.26), (ACARBOSE:Antidepressant therapy:0.42), (ACETAMINOPHEN\DIPHENHYDRAMINE:Antidepressant therapy:0.04), (ACETAMINOPHEN\PROPOXYPHENE HYDROCHLORIDE:Antidepressant therapy:0.08), (ACETAMINOPHENPROPOXYPHENE NAPSYLATE:Antidepressant therapy:0.09), (ADO-TRASTUZUMAB EMTANSINE:Antidepressant therapy:0.6), (AGALSIDASE BETA:Antidepressant therapy:0.58), (ALFACALCIDOL:Antidepressant therapy:0.38), (ALFUZOSIN HYDROCHLORIDE:Antidepressant therapy:0.64), (ALISKIREN HEMIFUMARATE:Antidepressant therapy:0.13), (AMLODIPINE BESYLATE\HYDROCHLOROTHIAZIDE\OLMESARTAN MEDOXOMIL:Antidepressant therapy:0.2), (AMLODIPINE BESYLATEOLMESARTAN MEDOXOMIL:Antidepressant therapy:0.15), (AMLODIPINE\VALSARTAN:Antidepressant therapy:0.39), (AMPICILLIN:Antidepressant therapy:0.05), (ANTIHISTAMINES NOS:Antidepressant therapy:0.13), (ARIPIPRAZOLE LAUROXIL:Antidepressant therapy:0.54), (AZELASTINE:Antidepressant therapy:0.48), (BECLOMETHASONE\FORMOTEROL:Antidepressant therapy:0.14), (BETAMETHASONE DIPROPIONATE:Antidepressant therapy:0.03), (BETAMETHASONE\CALCIPOTRIENE:Antidepressant therapy:0.04), (BLINATUMOMAB:Antidepressant therapy:0.1), (BRINZOLAMIDE:Antidepressant therapy:0.72), (BUPIVACAINE HYDROCHLORIDE:Antidepressant therapy:0.14), (BUPRENORPHINE\NALOXONE:Antidepressant therapy:0.27), (CALCITONIN SALMON:Antidepressant therapy:0.37), (CALCIUM POLYCARBOPHIL:Antidepressant therapy:0), (CALCIUM\MAGNESIUM:Antidepressant therapy:0.11), (CANAKINUMAB:Antidepressant therapy:0.45), (CAPSAICIN:Antidepressant therapy:0.02), (CARFILZOMIB:Antidepressant therapy:0.88), (CARVEDILOL PHOSPHATE:Antidepressant therapy:1.27), (CEFUROXIME AXETIL:Antidepressant therapy:0.12), (CHLORDIAZEPOXIDE HYDROCHLORIDE\CLIDINIUM BROMIDE:Antidepressant therapy:0.32), (CICLESONIDE:Antidepressant therapy:0.65), (CILASTATIN SODIUM\IMIPENEM:Antidepressant therapy:0.59), (CINACALCET HYDROCHLORIDE:Antidepressant therapy:0.1), (CODEINE PHOSPHATE\GUAIFENESIN:Antidepressant therapy:0.01), (CORTICOTROPIN:Antidepressant therapy:0.56), (CRIZOTINIB:Antidepressant therapy:0.23), (CYPROHEPTADINE:Antidepressant therapy:0.89), (DACLIZUMAB:Antidepressant therapy:0.92), (DALTEPARIN:Antidepressant therapy:0.62), (DANAZOL:Antidepressant therapy:0.41), (DEXTROAMPHETAMINE SULFATE:Antidepressant therapy:0.45), (DIGITOXIN:Antidepressant therapy:0.09), (DRONEDARONE:Antidepressant therapy:0.22), (DULAGLUTIDE:Antidepressant therapy:0.36), (DUTASTERIDE\TAMSULOSIN HYDROCHLORIDE:Antidepressant therapy:0.33), (EDOXABAN TOSYLATE:Antidepressant therapy:0.39), (EMPAGLIFLOZIN\LINAGLIPTIN:Antidepressant therapy:0.73), (ESTRADIOL\NORETHINDRONE ACETATE:Antidepressant therapy:0.08), (ESTRONE\SUS SCROFA OVARY:Antidepressant therapy:0.61), (FERROUS GLUCONATE:Antidepressant therapy:0.27), (FEXOFENADINE\PSEUDOEPHEDRINE:Antidepressant therapy:0.41), (FISH OIL\OMEGA-3 FATTY ACIDS:Antidepressant therapy:0.5), (FLUOCINONIDE:Antidepressant therapy:0.49), (FLUTICASONE:Antidepressant therapy:1.11), (FLUVASTATIN SODIUM:Antidepressant therapy:0.06), (FORMOTEROL FUMARATE\MOMETASONE FUROATE:Antidepressant therapy:0.32), (GARLIC:Antidepressant therapy:0.24), (GENTAMICIN:Antidepressant therapy:0.46), (GLYCOPYRRONIUM:Antidepressant therapy:0.14), (GLYCOPYRRONIUM\INDACATEROL:Antidepressant therapy:1.16), (GRANISETRON:Antidepressant therapy:0.03), (GUANFACINE HYDROCHLORIDE:Antidepressant therapy:0.81), (HERBALS\TURMERIC:Antidepressant therapy:0.81), (HYDROCHLOROTHIAZIDE\TELMISARTAN:Antidepressant therapy:0.63), (HYDROCODONE BITARTRATE\IBUPROFEN:Antidepressant therapy:0.15), (HYDROCORTISONE ACETATE:Antidepressant therapy:0.23), (HYDROXYCHLOROQUINE DIPHOSPHATE:Antidepressant therapy:0.65), (IDELALISIB:Antidepressant therapy:0.39), (ILOPROST:Antidepressant therapy:0.39), (IMATINIB MESYLATE:Antidepressant therapy:1.35), (INSULIN DEGLUDEC:Antidepressant therapy:0.17), (INTERFERON:Antidepressant therapy:0.32), (INTERFERON ALFA-2A:Antidepressant therapy:0.35), (IPRATROPIUM:Antidepressant therapy:1.71), (LACTASE:Antidepressant therapy:0.28), (LACTOBACILLUS RHAMNOSUS:Antidepressant therapy:0.24), (LERCANIDIPINE HYDROCHLORIDE:Antidepressant therapy:0.99), (LIDOCAINE HYDROCHLORIDE:Antidepressant therapy:1.15), (MEPOLIZUMAB:Antidepressant therapy:0.07), (METFORMIN HYDROCHLORIDE\VILDAGLIPTIN:Antidepressant therapy:0.37), (MICONAZOLE NITRATE:Antidepressant therapy:0.21), (MIDODRINE:Antidepressant therapy:0.59), (MOXONIDINE:Antidepressant therapy:0.15), (MUPIROCIN\MUPIROCIN CALCIUM:Antidepressant therapy:0.58), (NADROPARIN CALCIUM:Antidepressant therapy:0.41), (NALOXONE:Antidepressant therapy:0.32), (NALOXONE HYDROCHLORIDE\TILIDINE HYDROCHLORIDE:Antidepressant therapy:0.04), (NATEGLINIDE:Antidepressant therapy:0.26), (NIRAPARIB:Antidepressant therapy:0.12), (OLODATEROL HYDROCHLORIDE\TIOTROPIUM BROMIDE MONOHYDRATE:Antidepressant therapy:0.8), (PAZOPANIB HYDROCHLORIDE:Antidepressant therapy:1.06), (PEMETREXED DISODIUM:Antidepressant therapy:0.55), (PENICILLIN V:Antidepressant therapy:0.02), (PIMECROLIMUS:Antidepressant therapy:0.01), (POLYETHYLENE GLYCOL 4000:Antidepressant therapy:0.02), (PROPAFENONE HYDROCHLORIDE:Antidepressant therapy:0.26), (PROTRIPTYLINE HYDROCHLORIDE:Antidepressant therapy:0.16), (REGORAFENIB:Antidepressant therapy:0.22), (RETINOL:Antidepressant therapy:0.13), (SALMETEROL:Antidepressant therapy:0.29), (SODIUM FLUORIDE:Antidepressant therapy:0.29), (TERAZOSIN HYDROCHLORIDE:Antidepressant therapy:0.25), (TERBUTALINE SULFATE:Antidepressant therapy:0.96), (TETRABENAZINE:Antidepressant therapy:0.23), (TOCOPHEROL:Antidepressant therapy:0.4), (TRANDOLAPRIL:Antidepressant therapy:0.08), (TRIMIPRAMINE MALEATE:Antidepressant therapy:0.35), (TROSPIUM CHLORIDE:Antidepressant therapy:0.45), (UMECLIDINIUM BROMIDE:Antidepressant therapy:0.3), (UMECLIDINIUM BROMIDE\VILANTEROL TRIFENATATE:Antidepressant therapy:1.2), (VARDENAFIL HYDROCHLORIDE TRIHYDRATE:Antidepressant therapy:0.04), (VINCRISTINE SULFATE:Antidepressant therapy:0.69), (VORICONAZOLE:Antidepressant therapy:0.31), (ADO-TRASTUZUMAB EMTANSINE:Antiemetic supportive care:1.31), (ALCOHOL:Antiemetic supportive care:0.52), (ALFACALCIDOL:Antiemetic supportive care:0.35), (AMISULPRIDE:Antiemetic supportive care:0.18), (ANASTROZOLE:Antiemetic supportive care:1.71), (ASPARAGINASE:Antiemetic supportive care:0.38), (ATRACURIUM:Antiemetic supportive care:0.37), (ATRACURIUM BESYLATE:Antiemetic supportive care:0.06), (BENAZEPRIL HYDROCHLORIDE:Antiemetic supportive care:0.15), (BISACODYL OR DOCUSATE SODIUM:Antiemetic supportive care:0.59), (BUPRENORPHINE HYDROCHLORIDE:Antiemetic supportive care:0.59), (BUPROPION HYDROCHLORIDE:Antiemetic supportive care:0.93), (CALCITONIN SALMON:Antiemetic supportive care:0.32), (CALCIUM CARBONATE\VITAMIN D:Antiemetic supportive care:0.01), (CANAKINUMAB:Antiemetic supportive care:0.2), (CANDESARTAN CILEXETIL\HYDROCHLOROTHIAZIDE:Antiemetic supportive care:0.75), (CEFUROXIME:Antiemetic supportive care:0.79), (CEPHALEXIN:Antiemetic supportive care:1.54), (CERTOLIZUMAB PEGOL:Antiemetic supportive care:0.79), (CISATRACURIUM:Antiemetic supportive care:0.4), (CLINDAMYCIN:Antiemetic supportive care:0.54), (CORTISONE\HYDROCORTISONE:Antiemetic supportive care:0.58), (DIVALPROEX SODIUM:Antiemetic supportive care:0.84), (DOLASETRON MESYLATE:Antiemetic supportive care:0.03), (DOPAMINE HYDROCHLORIDE:Antiemetic supportive care:0.13), (DULOXETINE:Antiemetic supportive care:0.37), (ECONAZOLE:Antiemetic supportive care:0.23), (ELOTUZUMAB:Antiemetic supportive care:0.32), (ENALAPRIL MALEATE:Antiemetic supportive care:0.61), (ERIBULIN:Antiemetic supportive care:0.44), (ESZOPICLONE:Antiemetic supportive care:0.76), (ETORICOXIB:Antiemetic supportive care:0.06), (EVOLOCUMAB:Antiemetic supportive care:0.08), (FERROUS SULFATE\FERROUS SULFATE, DRIED:Antiemetic supportive care:1.46), (FESOTERODINE FUMARATE:Antiemetic supportive care:0.02), (FLUTICASONE\FLUTICASONE PROPIONATE:Antiemetic supportive care:0.06), (FORMOTEROL FUMARATE:Antiemetic supportive care:0.09), (FOSPHENYTOIN\FOSPHENYTOIN SODIUM:Antiemetic supportive care:0.04), (FUSIDIC ACID:Antiemetic supportive care:0.02), (GLYCOPYRRONIUM\INDACATEROL:Antiemetic supportive care:0.23), (HYDROCHLOROTHIAZIDE\IRBESARTAN:Antiemetic supportive care:0.26), (IBRITUMOMAB TIUXETAN:Antiemetic supportive care:0.84), (ILOPROST:Antiemetic supportive care:0.13), (INFLUENZA VIRUS VACCINE:Antiemetic supportive care:0.26), (IPRATROPIUM:Antiemetic supportive care:0.7), (IRON:Antiemetic supportive care:0.84), (ISOFLURANE:Antiemetic supportive care:0.69), (LACOSAMIDE:Antiemetic supportive care:0.17), (LAPATINIB DITOSYLATE:Antiemetic supportive care:0.23), (LATANOPROST:Antiemetic supportive care:0.22), (LEDIPASVIR\SOFOSBUVIR:Antiemetic supportive care:0.32), (LERCANIDIPINE:Antiemetic supportive care:0.84), (LERCANIDIPINE HYDROCHLORIDE:Antiemetic supportive care:0.46), (LIDOCAINE\PRILOCAINE:Antiemetic supportive care:0.52), (LINAGLIPTIN:Antiemetic supportive care:0.41), (LITHIUM CARBONATE:Antiemetic supportive care:0.36), (LOMUSTINE:Antiemetic supportive care:0.05), (LOVASTATIN:Antiemetic supportive care:0.67), (METHENAMINE HIPPURATE:Antiemetic supportive care:0.05), (METHENOLONE ENANTHATE:Antiemetic supportive care:0), (MINOCYCLINE\MINOCYCLINE HYDROCHLORIDE:Antiemetic supportive care:0.24), (MOXIFLOXACIN HYDROCHLORIDE:Antiemetic supportive care:1.22), (NALOXONE:Antiemetic supportive care:0), (NICARDIPINE:Antiemetic supportive care:0.08), (NILOTINIB:Antiemetic supportive care:1.79), (NOREPINEPHRINE:Antiemetic supportive care:0.4), (OBINUTUZUMAB:Antiemetic supportive care:0.13), (OLAPARIB:Antiemetic supportive care:0.58), (OSELTAMIVIR:Antiemetic supportive care:0.87), (OSIMERTINIB:Antiemetic supportive care:0.02), (OXCARBAZEPINE:Antiemetic supportive care:1.54), (PALBOCICLIB:Antiemetic supportive care:0.04), (PAZOPANIB HYDROCHLORIDE:Antiemetic supportive care:0.82), (PENICILLIN:Antiemetic supportive care:0.73), (PENICILLIN V:Antiemetic supportive care:0.2), (PENTAMIDINE ISETHIONATE:Antiemetic supportive care:0.11), (PENTOSTATIN:Antiemetic supportive care:0.14), (PHENOBARBITAL:Antiemetic supportive care:0.7), (PITAVASTATIN CALCIUM:Antiemetic supportive care:0.15), (PONATINIB:Antiemetic supportive care:0.69), (PRAMIPEXOLE:Antiemetic supportive care:0.15), (PRAMIPEXOLE DIHYDROCHLORIDE:Antiemetic supportive care:0.1), (RANOLAZINE:Antiemetic supportive care:0.18), (ROFLUMILAST:Antiemetic supportive care:0.32), (ROPIVACAINE:Antiemetic supportive care:0.65), (SACUBITRIL\VALSARTAN:Antiemetic supportive care:0.33), (SECUKINUMAB:Antiemetic supportive care:0.26), (SOTALOL:Antiemetic supportive care:0.72), (SUFENTANIL CITRATE:Antiemetic supportive care:0.17), (TAMOXIFEN CITRATE:Antiemetic supportive care:0.13), (TAZOBACTAM:Antiemetic supportive care:0.35), (THIOPENTAL:Antiemetic supportive care:0.18), (TIMOLOL:Antiemetic supportive care:0.39), (TIOTROPIUM:Antiemetic supportive care:0.38), (TIZANIDINE HYDROCHLORIDE:Antiemetic supportive care:0.21), (TOCILIZUMAB:Antiemetic supportive care:0.5), (TOFACITINIB CITRATE:Antiemetic supportive care:0.36), (TRIAMCINOLONE ACETONIDE:Antiemetic supportive care:0.16), (VEMURAFENIB:Antiemetic supportive care:0.16), (VINDESINE SULFATE:Antiemetic supportive care:0.01), (VITAMIN B:Antiemetic supportive care:0.11), (.ALPHA.1-PROTEINASE INHIBITOR HUMAN:Antifungal prophylaxis:0.59), (ACEBUTOLOL HYDROCHLORIDE:Antifungal prophylaxis:0.09), (ACETAMINOPHEN\OXYCODONE\OXYCODONE HYDROCHLORIDE:Antifungal prophylaxis:0.76), (ACETAMINOPHEN\PROPOXYPHENE NAPSYLATE:Antifungal prophylaxis:0.16), (ACITRETIN:Antifungal prophylaxis:0.21), (ACTIVATED CHARCOAL:Antifungal prophylaxis:0.19), (ADENINE:Antifungal prophylaxis:0.05), (ADO-TRASTUZUMAB EMTANSINE:Antifungal prophylaxis:0.59), (AGALSIDASE BETA:Antifungal prophylaxis:0.34), (ALUMINUM HYDROXIDE\MAGNESIUM OXIDE\OXETHAZAINE:Antifungal prophylaxis:0.5), (ALUMINUM SILICATE:Antifungal prophylaxis:0.12), (AMANTADINE:Antifungal prophylaxis:0.26), (AMILORIDE HYDROCHLORIDE\HYDROCHLOROTHIAZIDE:Antifungal prophylaxis:0.32), (AMINO ACIDS\CARBOHYDRATES\MINERALS\SOYBEAN OIL\VITAMINS:Antifungal prophylaxis:0.66), (AMISULPRIDE:Antifungal prophylaxis:0.36), (AMLODIPINE\PERINDOPRIL:Antifungal prophylaxis:0.29), (AMLODIPINE\VALSARTAN:Antifungal prophylaxis:0.39), (AMMONIUM GLYCYRRHIZATE\GLYCINE\METHIONINE:Antifungal prophylaxis:0.12), (AMPHETAMINE ASPARTATE\AMPHETAMINE SULFATE\DEXTROAMPHETAMINE SACCHARATE\DEXTROAMPHETAMINE SULFATE:Antifungal prophylaxis:0.36), (AMPICILLIN SODIUM:Antifungal prophylaxis:0.07),(ARMODAFINIL:Antifungal prophylaxis:0.41), (ARSENIC TRIOXIDE:Antifungal prophylaxis:0.73), (ASPIRIN\MAGNESIUM OXIDE:Antifungal prophylaxis:0.41), (BENAZEPRIL\BENAZEPRIL HYDROCHLORIDE:Antifungal prophylaxis:0.6), (BENZBROMARONE:Antifungal prophylaxis:0.68), (BERAPROST:Antifungal prophylaxis:0.18), (BETAHISTINE MESILATE:Antifungal prophylaxis:0.41), (BICARBONATE ION:Antifungal prophylaxis:0.11), (BLESELUMAB:Antifungal prophylaxis:0.31), (BRINZOLAMIDE:Antifungal prophylaxis:0.67), (BUDESONIDE\FORMOTEROL:Antifungal prophylaxis:0.15), (BUDESONIDE\FORMOTEROL FUMARATE:Antifungal prophylaxis:0.64), (BUPRENORPHINE HYDROCHLORIDE\NALOXONE HYDROCHLORIDE:Antifungal prophylaxis:0.35), (CABAZITAXEL:Antifungal prophylaxis:0.48), (CALCITONIN:Antifungal prophylaxis:0.59), (CALCITONIN SALMON:Antifungal prophylaxis:1.08), (CALCIUM ASPARTATE:Antifungal prophylaxis:0.19), (CALCIUM CARBONATE\ERGOCALCIFEROL\RETINOL:Antifungal prophylaxis:0.14), (CALCIUM\MAGNESIUM:Antifungal prophylaxis:0.21), (CAMPHOR\MENTHOL\METHYL SALICYLATE:Antifungal prophylaxis:0.24), (CANAGLIFLOZIN\METFORMIN HYDROCHLORIDE:Antifungal prophylaxis:0.25), (CANAKINUMAB:Antifungal prophylaxis:1.04), (CARISOPRODOL:Antifungal prophylaxis:0.16), (CARVEDILOL PHOSPHATE:Antifungal prophylaxis:0.71), (CEFMETAZOLE:Antifungal prophylaxis:0.11), (CEFPROZIL:Antifungal prophylaxis:0.04), (CEPHARANTHINE:Antifungal prophylaxis:0.4), (CHLORAMPHENICOL:Antifungal prophylaxis:0.08), (CHLORPHENIRAMINE\HYDROCODONE:Antifungal prophylaxis:0.03), (CHLORTHALIDONE:Antifungal prophylaxis:0.55), (CHONDROITIN SULFATE (BOVINE):Antifungal prophylaxis:0.22), (CICLESONIDE:Antifungal prophylaxis:0.37), (CLOMIPRAMINE HYDROCHLORIDE:Antifungal prophylaxis:0.1), (COBICISTAT\DARUNAVIR ETHANOLATE:Antifungal prophylaxis:0.09), (CYCLOPHOSPHAMIDE\CYTARABINE\DEXAMETHASONE\DOXORU BICIN\METHOTREXATE\VINCRISTINE:Antifungal prophylaxis:0.86), (DABRAFENIB:Antifungal prophylaxis:0.78), (DAPAGLIFLOZIN:Antifungal prophylaxis:0.04), (DAPAGLIFLOZIN PROPANEDIOL:Antifungal prophylaxis:0.03), (DAUNORUBICIN CITRATE:Antifungal prophylaxis:0.25), (DESFLURANE:Antifungal prophylaxis:0.71), (DESONIDE:Antifungal prophylaxis:0.33), (DEXMEDETOMIDINE HYDROCHLORIDE:Antifungal prophylaxis:1.19), (DILAZEP:Antifungal prophylaxis:0.53), (DISTIGMINE BROMIDE:Antifungal prophylaxis:0.2), (DOCETAXEL:Antifungal prophylaxis:0.53), (DOFETILIDE:Antifungal prophylaxis:0.41), (DOLASETRON MESYLATE:Antifungal prophylaxis:0.03), (DOXYCYCLINE HYCLATE:Antifungal prophylaxis:0.09), (DUTASTERIDE\TAMSULOSIN HYDROCHLORIDE:Antifungal prophylaxis:0.37), (ECABET SODIUM:Antifungal prophylaxis:0.21), (EDARAVONE:Antifungal prophylaxis:0.25), (ELETRIPTAN HYDROBROMIDE:Antifungal prophylaxis:0.74), (EMTRICITABINE\RILPIVIRINE\TENOFOVIR DISOPROXIL FUMARATE:Antifungal prophylaxis:0.27), (ESMOLOL HYDROCHLORIDE:Antifungal prophylaxis:0.21), (ESTROGENS, CONJUGATED\MEDROXYPROGESTERONE ACETATE:Antifungal prophylaxis:0.38), (ETORICOXIB:Antifungal prophylaxis:1), (EVOLOCUMAB:Antifungal prophylaxis:0.29), (FERRIC CARBOXYMALTOSE:Antifungal prophylaxis:0.03), (FERROUS GLUCONATE:Antifungal prophylaxis:0.19), (FERROUS SULFATE ANHYDROUS:Antifungal prophylaxis:0.23), (FERROUS SULFATE\GLYCINE:Antifungal prophylaxis:0.15), (FLUOCINOLONE ACETONIDE:Antifungal prophylaxis:0.22), (FLUOCINONIDE:Antifungal prophylaxis:0.13), (FLUTICASONE PROPIONATE\FORMOTEROL FUMARATE:Antifungal prophylaxis:0.13), (FORMOTEROL FUMARATE DIHYDRATE\MOMETASONE FUROATE:Antifungal prophylaxis:0.74), (FORMOTEROL FUMARATE\MOMETASONE FUROATE:Antifungal prophylaxis:0.23), (FOSPHENYTOIN\FOSPHENYTOIN SODIUM:Antifungal prophylaxis:0.8), (FUSIDIC ACID:Antifungal prophylaxis:1.16), (GIMERACIL\OTERACIL\TEGAFUR:Antifungal prophylaxis:1.02), (GLYCOPYRRONIUM:Antifungal prophylaxis:0.22), (GUSPERIMUS TRIHYDROCHLORIDE:Antifungal prophylaxis:1.04), (HYDROCHLOROTHIAZIDE\IRBESARTAN:Antifungal prophylaxis:1.09), (HYDROCORTISONE HEMISUCCINATE:Antifungal prophylaxis:0.1), (HYDROXYCHLOROQUINE DIPHOSPHATE:Antifungal prophylaxis:0.13), (IBRITUMOMAB TIUXETAN:Antifungal prophylaxis:2.7), (ICOSAPENT:Antifungal prophylaxis:0.05), (IDARUCIZUMAB:Antifungal prophylaxis:0.02), (ILOPROST:Antifungal prophylaxis:0.82), (IMIPRAMINE HYDROCHLORIDE:Antifungal prophylaxis:0.64), (INDAPAMIDE\PERINDOPRIL:Antifungal prophylaxis:1.67), (INFLIXIMAB-DYYB:Antifungal prophylaxis:0.11), (INTERFERON ALFA-2A:Antifungal prophylaxis:0.57), (INTERFERON GAMMA-1B:Antifungal prophylaxis:0.83), (ISOFLURANE:Antifungal prophylaxis:0.33), (LANDIOLOL HYDROCHLORIDE:Antifungal prophylaxis:0.16), (LEVOCABASTINE HYDROCHLORIDE:Antifungal prophylaxis:0.03), (LEVOLEUCOVORIN CALCIUM:Antifungal prophylaxis:0.88), (LITHIUM CARBONATE:Antifungal prophylaxis:1.15), (LOXOPROFEN SODIUM DIHYDRATE:Antifungal prophylaxis:0.44), (LULICONAZOLE:Antifungal prophylaxis:0.04), (LYMPHOCYTES:Antifungal prophylaxis:0.24), (MAGNESIUM ASPARTATE HYDROCHLORIDE:Antifungal prophylaxis:0.29), (MAGNESIUM ASPARTATE\POTASSIUM ASPARTATE:Antifungal prophylaxis:0.39), (MAXACALCITOL:Antifungal prophylaxis:0.37), (MECLIZINE HYDROCHLORIDE:Antifungal prophylaxis:0.2), (MECLIZINE\MECLIZINE HYDROCHLORIDE:Antifungal prophylaxis:0.09), (MEPOLIZUMAB:Antifungal prophylaxis:0.07), (METFORMIN PAMOATE:Antifungal prophylaxis:0.01), (METHENAMINE HIPPURATE:Antifungal prophylaxis:0.44), (METHENOLONE ACETATE:Antifungal prophylaxis:0.12), (METHIMAZOLE:Antifungal prophylaxis:1.16), (METHOTREXATE\METHOTREXATE SODIUM:Antifungal prophylaxis:0.42), (MEXILETINE HYDROCHLORIDE:Antifungal prophylaxis:0.5), (MODAFINIL:Antifungal prophylaxis:0.2), (MOSAPRIDE CITRATE DIHYDRATE:Antifungal prophylaxis:0.12), (MOXONIDINE:Antifungal prophylaxis:0.36), (NABUMETONE:Antifungal prophylaxis:0.3), (NALOXONE HYDROCHLORIDE\TILIDINE HYDROCHLORIDE:Antifungal prophylaxis:0.92), (NOREPINEPHRINE:Antifungal prophylaxis:1.32), (NOREPINEPHRINE BITARTRATE:Antifungal prophylaxis:0.59), (OLODATEROL HYDROCHLORIDE\TIOTROPIUM BROMIDE MONOHYDRATE:Antifungal prophylaxis:0.68), (OXYBUTYNIN CHLORIDE:Antifungal prophylaxis:0.65), (PANITUMUMAB:Antifungal prophylaxis:0.3), (PANTOPRAZOLE MAGNESIUM:Antifungal prophylaxis:1.27), (PARICALCITOL:Antifungal prophylaxis:0.1), (PAROXETINE:Antifungal prophylaxis:0.57), (PAZUFLOXACIN MESILATE:Antifungal prophylaxis:0.29), (PEGINTERFERON ALFA-2B:Antifungal prophylaxis:0.45), (PENICILLIN V POTASSIUM:Antifungal prophylaxis:0.04), (PENTOXIFYLLINE:Antifungal prophylaxis:0.33), (PHENOBARBITAL SODIUM:Antifungal prophylaxis:0.14), (PIMOBENDAN:Antifungal prophylaxis:0.54), (PIROXICAM:Antifungal prophylaxis:0.2), (PNEUMOCOCCAL VACCINE, POLYVALENT 23:Antifungal prophylaxis:0.05), (POTASSIUM CITRATE\TRISODIUM CITRATE DIHYDRATE:Antifungal prophylaxis:1.24), (PRAMIPEXOLE DIHYDROCHLORIDE:Antifungal prophylaxis:0.66), (PRIMIDONE:Antifungal prophylaxis:1.2), (PRISTINAMYCIN:Antifungal prophylaxis:0.31), (QUINACRINE:Antifungal prophylaxis:0.09), (RALOXIFENE HYDROCHLORIDE:Antifungal prophylaxis:0.31), (RAMUCIRUMAB:Antifungal prophylaxis:0.59), (RANOLAZINE:Antifungal prophylaxis:1.26), (REGORAFENIB:Antifungal prophylaxis:0.19), (RETINOL:Antifungal prophylaxis:0.54), (RILMENIDINE PHOSPHATE:Antifungal prophylaxis:0.09), (RIVASTIGMINE:Antifungal prophylaxis:0.14), (RIVASTIGMINE TARTRATE:Antifungal prophylaxis:0.31), (ROFLUMILAST:Antifungal prophylaxis:1.28), (SACUBITRIL\VALSARTAN:Antifungal prophylaxis:0.47), (SALMETEROL:Antifungal prophylaxis:0.92), (SARPOGRELATE HYDROCHLORIDE:Antifungal prophylaxis:0.3), (SECUKINUMAB:Antifungal prophylaxis:0.99), (SITAFLOXACIN:Antifungal prophylaxis:0.02), (SODIUM OXYBATE:Antifungal prophylaxis:0.25), (SUCCINYLCHOLINE CHLORIDE:Antifungal prophylaxis:0.13), (SUFENTANIL:Antifungal prophylaxis:0.1), (SUFENTANIL CITRATE:Antifungal prophylaxis:0.31), (SUMATRIPTAN\SUMATRIPTAN SUCCINATE:Antifungal prophylaxis:1.02), (SUNITINIB MALATE:Antifungal prophylaxis:0.18), (SUPLATAST TOSILATE:Antifungal prophylaxis:0.22), (TEMOCAPRIL HYDROCHLORIDE:Antifungal prophylaxis:0.4), (TENELIGLIPTIN:Antifungal prophylaxis:0.31), (TENIPOSIDE:Antifungal prophylaxis:0.32), (TERAZOSIN\TERAZOSIN HYDROCHLORIDE:Antifungal prophylaxis:0.02), (TERBUTALINE:Antifungal prophylaxis:0.68), (TETRACYCLINE:Antifungal prophylaxis:0.98), (THEOPHYLLINE:Antifungal prophylaxis:0.02), (THROMBOPOIETIN:Antifungal prophylaxis:0.16), (TOLVAPTAN:Antifungal prophylaxis:0.15), (TROSPIUM CHLORIDE:Antifungal prophylaxis:0.41), (UMECLIDINIUM BROMIDE\VILANTEROL TRIFENATATE:Antifungal prophylaxis:0.17), (VADASTUXIMAB TALIRINE:Antifungal prophylaxis:0.08), (VARENICLINE TARTRATE:Antifungal prophylaxis:0.46), (VASOPRESSIN:Antifungal prophylaxis:0.25), (VECURONIUM BROMIDE:Antifungal prophylaxis:0.38), (VINDESINE SULFATE:Antifungal prophylaxis:1.46), (VINORELBINE TARTRATE:Antifungal prophylaxis:0.09), (VINORELBINE\VINORELBINE TARTRATE:Antifungal prophylaxis:0.19), (ZINC:Antifungal prophylaxis:0.47), (ACETAMINOPHEN\CODEINE PHOSPHATE:Antifungal treatment:0.64), (ALBUTEROL SULFATE\IPRATROPIUM BROMIDE:Antifungal treatment:0.23), (ALCOHOL:Antifungal treatment:0.01), (AMIKACIN SULFATE:Antifungal treatment:0.12), (AMIODARONE HYDROCHLORIDE:Antifungal treatment:0.3), (BISACODYL OR DOCUSATE SODIUM:Antifungal treatment:0.35), (BISOPROLOL FUMARATE:Antifungal treatment:0.18), (BLINATUMOMAB:Antifungal treatment:0.92), (BUPRENORPHINE HYDROCHLORIDE\NALOXONE HYDROCHLORIDE:Antifungal treatment:0.03), (CALCIUM ACETATE:Antifungal treatment:0.14), (CANAKINUMAB:Antifungal treatment:0), (CANDESARTAN CILEXETIL\HYDROCHLOROTHIAZIDE:Antifungal treatment:0.09), (CERTOLIZUMAB PEGOL:Antifungal treatment:0.42), (CHLORAMBUCIL:Antifungal treatment:0.29), (CHOLESTYRAMINE:Antifungal treatment:0.34), (COLISTIMETHATE SODIUM:Antifungal treatment:0.09), (CYCLOBENZAPRINE:Antifungal treatment:0.26), (DACLIZUMAB:Antifungal treatment:0.12), (DALFAMPRIDINE:Antifungal treatment:0.09), (DEXLANSOPRAZOLE:Antifungal treatment:0.66), (DILTIAZEM HYDROCHLORIDE:Antifungal treatment:0.46), (DIVALPROEX SODIUM:Antifungal treatment:0.75), (ENALAPRIL:Antifungal treatment:1.35), (ERYTHROMYCIN:Antifungal treatment:0.16), (EVOLOCUMAB:Antifungal treatment:0.14), (EZETIMIBE:Antifungal treatment:0.19), (FLECAINIDE:Antifungal treatment:0.03), (GLIMEPIRIDE:Antifungal treatment:0.1), (IBRITUMOMAB TIUXETAN:Antifungal treatment:0.36), (ILOPROST:Antifungal treatment:0.21), (IMATINIB MESYLATE:Antifungal treatment:0.32), (INTERFERON GAMMA-1B:Antifungal treatment:0.11), (IPRATROPIUM:Antifungal treatment:0.2), (LERCANIDIPINE:Antifungal treatment:0.06), (LIDOCAINE\PRILOCAINE:Antifungal treatment:0.14), (LINAGLIPTIN:Antifungal treatment:0), (LOVASTATIN:Antifungal treatment:0.53), (METAXALONE:Antifungal treatment:0.18), (MOXIFLOXACIN HYDROCHLORIDE:Antifungal treatment:0.44), (NEBIVOLOL:Antifungal treatment:0.41), (NILOTINIB:Antifungal treatment:0.92), (NITROFURANTOIN:Antifungal treatment:0.27), (OXCARBAZEPINE:Antifungal treatment:1.23), (PAROXETINE:Antifungal treatment:0.01), (PEGFILGRASTIM:Antifungal treatment:0.31), (PRIMIDONE:Antifungal treatment:0.18), (QUETIAPINE:Antifungal treatment:0), (RANOLAZINE:Antifungal treatment:0.39), (SACUBITRIL\VALSARTAN:Antifungal treatment:0.39), (SODIUM OXYBATE:Antifungal treatment:0.11), (SOTALOL:Antifungal treatment:0.19), (SULFAMETHOXAZOLE:Antifungal treatment:0.44), (TERIFLUNOMIDE:Antifungal treatment:0.21), (TETRACYCLINE:Antifungal treatment:0.12), (THALIDOMIDE:Antifungal treatment:1.07), (TOCILIZUMAB:Antifungal treatment:0.05), (TORSEMIDE:Antifungal treatment:0.46), (VARENICLINE TARTRATE:Antifungal treatment:0.04), (VERAPAMIL HYDROCHLORIDE:Antifungal treatment:0.53), (VINCRISTINE SULFATE:Antifungal treatment:1.13), (ZOLPIDEM\ZOLPIDEM TARTRATE:Antifungal treatment:0.32), (.ALPHA.1-PROTEINASE INHIBITOR HUMAN:Antiinflammatory therapy:0.01), (ACETAMINOPHEN\PROPOXYPHENE HYDROCHLORIDE:Antiinflammatory therapy:0.18), (ACETAMINOPHEN\PROPOXYPHENE NAPSYLATE:Antiinflammatory therapy:0.19), (ADO-TRASTUZUMAB EMTANSINE:Antiinflammatory therapy:0.48), (ALENDRONIC ACID:Antiinflammatory therapy:0.52), (ALUMINUM HYDROXIDE\DIMETHICONE\MAGNESIUM HYDROXIDE:Antiinflammatory therapy:0.15), (AMISULPRIDE:Antiinflammatory therapy:0.29), (AMLODIPINE\VALSARTAN:Antiinflammatory therapy:0.22), (ANTIHISTAMINES NOS:Antiinflammatory therapy:0.02), (ATOVAQUONE:Antiinflammatory therapy:0.21), (ATRACURIUM:Antiinflammatory therapy:0.31), (BENAZEPRIL\BENAZEPRIL HYDROCHLORIDE:Antiinflammatory therapy:0.66), (BETAMETHASONE DIPROPIONATE:Antiinflammatory therapy:0.07), (BETAMETHASONE\CALCIPOTRIENE:Antiinflammatory therapy:0.01), (BIFIDOBACTERIUM LONGUM SUBSP. INFANTIS:Antiinflammatory therapy:0.21), (BUPRENORPHINE HYDROCHLORIDE\NALOXONE HYDROCHLORIDE:Antiinflammatory therapy:0.89), (CALCITONIN:Antiinflammatory therapy:0.04), (CALCIUM\MAGNESIUM:Antiinflammatory therapy:0.03), (CALCIUM\MAGNESIUM\ZINC:Antiinflammatory therapy:0.16), (CANAGLIFLOZIN\METFORMIN HYDROCHLORIDE:Antiinflammatory therapy:0.4), (CANDESARTAN CILEXETIL\HYDROCHLOROTHIAZIDE:Antiinflammatory therapy:1.11), (CARVEDILOL PHOSPHATE:Antiinflammatory therapy:1.12), (CHLORAMBUCIL:Antiinflammatory therapy:0.04), (CHLORDIAZEPOXIDE HYDROCHLORIDE\CLIDINIUM BROMIDE:Antiinflammatory therapy:0.23), (CHLORHEXIDINE:Antiinflammatory therapy:0.18), (COD LIVER OIL:Antiinflammatory therapy:0.2), (CORTICOTROPIN:Antiinflammatory therapy:0.26), (CRIZOTINIB:Antiinflammatory therapy:0.36), (DABIGATRAN:Antiinflammatory therapy:0.05), (DACLIZUMAB:Antiinflammatory therapy:1.28), (DALTEPARIN:Antiinflammatory therapy:0.44), (DANAZOL:Antiinflammatory therapy:0.09), (DASATINIB:Antiinflammatory therapy:1.15), (DESFLURANE:Antiinflammatory therapy:0.58), (DIPYRONE:Antiinflammatory therapy:0.69), (DOCUSATE CALCIUM:Antiinflammatory therapy:0.71), (DOFETILIDE:Antiinflammatory therapy:0.45), (ECONAZOLE:Antiinflammatory therapy:0.13), (EMPAGLIFLOZIN\LINAGLIPTIN:Antiinflammatory therapy:0.66), (ESMOLOL HYDROCHLORIDE:Antiinflammatory therapy:0.34), (ESTROGENS:Antiinflammatory therapy:0.17), (ESTROGENS, CONJUGATED\MEDROXYPROGESTERONE ACETATE:Antiinflammatory therapy:0.84), (EVOLOCUMAB:Antiinflammatory therapy:1.5), (FENOFIBRIC ACID:Antiinflammatory therapy:0.44), (FEXOFENADINE\PSEUDOEPHEDRINE:Antiinflammatory therapy:0.03), (FISH OIL\OMEGA-3 FATTY ACIDS:Antiinflammatory therapy:0.24), (FLECAINIDE:Antiinflammatory therapy:0.99), (FORMOTEROL:Antiinflammatory therapy:0.15), (FORMOTEROL FUMARATE:Antiinflammatory therapy:0.67), (FORMOTEROL FUMARATE DIHYDRATE\MOMETASONE FUROATE:Antiinflammatory therapy:0.52), (FORMOTEROL FUMARATE\MOMETASONE FUROATE:Antiinflammatory therapy:0.21), (FULVESTRANT:Antiinflammatory therapy:0.49), (GLYCOPYRROLATE:Antiinflammatory therapy:0.39), (GLYCOPYRRONIUM\INDACATEROL:Antiinflammatory therapy:0.44), (HYALURONATE SODIUM:Antiinflammatory therapy:0.11), (HYDROCHLOROTHIAZIDE\IRBESARTAN:Antiinflammatory therapy:0.74), (HYDROCHLOROTHIAZIDE\LOSARTAN:Antiinflammatory therapy:0.27), (HYDROCHLOROTHIAZIDE\TELMISARTAN:Antiinflammatory therapy:0.34), (HYDROXYCHLOROQUINE DIPHOSPHATE:Antiinflammatory therapy:0.53), (HYOSCYAMINE:Antiinflammatory therapy:0.72), (INDAPAMIDE\PERINDOPRIL:Antiinflammatory therapy:0.51), (INFLIXIMAB-DYYB:Antiinflammatory therapy:0.02), (INSULIN BEEF:Antiinflammatory therapy:0.02), (INSULIN DEGLUDEC:Antiinflammatory therapy:0.04), (IODINE:Antiinflammatory therapy:0.12), (IPRATROPIUM:Antiinflammatory therapy:1.22), (IRINOTECAN HYDROCHLORIDE:Antiinflammatory therapy:0.37), (ISOFLURANE:Antiinflammatory therapy:0.63), (ITRACONAZOLE:Antiinflammatory therapy:0.48), (IXAZOMIB:Antiinflammatory therapy:0.01), (LABETALOL HYDROCHLORIDE:Antiinflammatory therapy:0.4), (LABETALOL\LABETALOL HYDROCHLORIDE:Antiinflammatory therapy:1.11), (LACTASE:Antiinflammatory therapy:0.05), (LACTOBACILLUS RHAMNOSUS:Antiinflammatory therapy:0.05), (LEDIPASVIR\SOFOSBUVIR:Antiinflammatory therapy:0.7), (LERCANIDIPINE HYDROCHLORIDE:Antiinflammatory therapy:0.65), (LEVOLEUCOVORIN CALCIUM:Antiinflammatory therapy:0.14), (LIDOCAINE\PRILOCAINE:Antiinflammatory therapy:0.41), (LINAGLIPTIN:Antiinflammatory therapy:1.14), (MIDODRINE:Antiinflammatory therapy:0.08), (MINOCYCLINE\MINOCYCLINE HYDROCHLORIDE:Antiinflammatory therapy:1.08), (NADROPARIN CALCIUM:Antiinflammatory therapy:0.12), (NALOXONE:Antiinflammatory therapy:0.02), (NEOSTIGMINE:Antiinflammatory therapy:0.88), (NOREPINEPHRINE:Antiinflammatory therapy:0.34), (NORETHINDRONE:Antiinflammatory therapy:0.54), (OCTREOTIDE ACETATE:Antiinflammatory therapy:0.11), (OLODATEROL HYDROCHLORIDE\TIOTROPIUM BROMIDE MONOHYDRATE:Antiinflammatory therapy:0.63), (ONABOTULINUMTOXINA:Antiinflammatory therapy:0.16), (OXAZEPAM:Antiinflammatory therapy:0.37), (PAMIDRONATE DISODIUM:Antiinflammatory therapy:0.39), (PANTOPRAZOLE MAGNESIUM:Antiinflammatory therapy:1.33), (PAROXETINE:Antiinflammatory therapy:0.94), (PEGINTERFERON BETA-1A:Antiinflammatory therapy:0.43), (PEMETREXED:Antiinflammatory therapy:0.17), (PENICILLIN G:Antiinflammatory therapy:0.08), (PHENYTOIN SODIUM:Antiinflammatory therapy:0.15), (PRAMIPEXOLE:Antiinflammatory therapy:0.76), (PRAZOSIN HYDROCHLORIDE:Antiinflammatory therapy:0.26), (QUINAPRIL:Antiinflammatory therapy:0.03), (RADIATION THERAPY:Antiinflammatory therapy:0.23), (RALOXIFENE HYDROCHLORIDE:Antiinflammatory therapy:0.53), (REGORAFENIB:Antiinflammatory therapy:0.2), (RETINOL:Antiinflammatory therapy:0.27), (RIZATRIPTAN:Antiinflammatory therapy:0.05), (ROFLUMILAST:Antiinflammatory therapy:0.85), (ROPIVACAINE:Antiinflammatory therapy:0.23), (SACCHAROMYCES CEREVISIAE:Antiinflammatory therapy:0.04), (SACUBITRIL\VALSARTAN:Antiinflammatory therapy:0.77), (SITAGLIPTIN:Antiinflammatory therapy:0.08), (SUNITINIB MALATE:Antiinflammatory therapy:0.31), (THIOPENTAL:Antiinflammatory therapy:0.08), (TIMOLOL:Antiinflammatory therapy:0.94), (TIMOLOL MALEATE:Antiinflammatory therapy:0.34), (VANCOMYCIN HYDROCHLORIDE:Antiinflammatory therapy:0.73), (ACETAMINOPHEN\CODEINE PHOSPHATE:Antiphospholipid syndrome:0.25), (AMIODARONE:Antiphospholipid syndrome:0.16), (AMIODARONE HYDROCHLORIDE:Antiphospholipid syndrome:0.14), (CALCIUM:Antiphospholipid syndrome:0.75), (CERTOLIZUMAB PEGOL:Antiphospholipid syndrome:0.09), (DEXLANSOPRAZOLE:Antiphospholipid syndrome:0.52), (DIVALPROEX SODIUM:Antiphospholipid syndrome:0.68), (ESZOPICLONE:Antiphospholipid syndrome:0.12), (EVOLOCUMAB:Antiphospholipid syndrome:0.32), (IBRUTINIB:Antiphospholipid syndrome:0.31), (INDOMETHACIN:Antiphospholipid syndrome:0.03), (LOVASTATIN:Antiphospholipid syndrome:0.44), (POTASSIUM:Antiphospholipid syndrome:0.55), (SECUKINUMAB:Antiphospholipid syndrome:0.13), (SELEXIPAG:Antiphospholipid syndrome:0.21), (TORSEMIDE:Antiphospholipid syndrome:0.29), (TRAZODONE HYDROCHLORIDE:Antiphospholipid syndrome:0.45), (VERAPAMIL HYDROCHLORIDE:Antiphospholipid syndrome:0.16), (ACEBUTOLOL HYDROCHLORIDE:Antiplatelet therapy:0.19), (ACETAMINOPHEN\BUTALBITAL\CAFFEINE:Antiplatelet therapy:0.54), (ACETAMINOPHEN\OXYCODONE\OXYCODONE HYDROCHLORIDE:Antiplatelet therapy:0.3), (ACETAZOLAMIDE:Antiplatelet therapy:0.49), (ACTIVATED CHARCOAL:Antiplatelet therapy:0.07), (ADO-TRASTUZUMAB EMTANSINE:Antiplatelet therapy:0), (AGALSIDASE BETA:Antiplatelet therapy:0.62), (AMANTADINE:Antiplatelet therapy:0.31), (AMISULPRIDE:Antiplatelet therapy:0.22), (AMLODIPINE BESYLATE\HYDROCHLOROTHIAZIDE\VALSARTAN:Antiplatelet therapy:0.26), (AMPHETAMINE ASPARTATE\AMPHETAMINE SULFATE\DEXTROAMPHETAMINE SACCHARATE\DEXTROAMPHETAMINE SULFATE:Antiplatelet therapy:0.7), (AMPHOTERICIN B:Antiplatelet therapy:0.38), (ATRACURIUM:Antiplatelet therapy:0.15), (ATROPINE SULFATE\DIPHENOXYLATE HYDROCHLORIDE:Antiplatelet therapy:0.14), (BENAZEPRIL\BENAZEPRIL HYDROCHLORIDE:Antiplatelet therapy:0.59), (BOCOCIZUMAB:Antiplatelet therapy:0.03), (BRINZOLAMIDE:Antiplatelet therapy:0.23), (BUDESONIDE\FORMOTEROL FUMARATE:Antiplatelet therapy:0.49), (CANAGLIFLOZIN\METFORMIN HYDROCHLORIDE:Antiplatelet therapy:0.65), (CARBIMAZOLE:Antiplatelet therapy:0.08), (CEFTRIAXONE SODIUM:Antiplatelet therapy:0.87), (CERTOLIZUMAB PEGOL:Antiplatelet therapy:1.27), (CILASTATIN SODIUM\IMIPENEM:Antiplatelet therapy:0.77), (CIMETIDINE:Antiplatelet therapy:0.62), (CLINDAMYCIN\CLINDAMYCIN PHOSPHATE:Antiplatelet therapy:1.56), (CLOMIPRAMINE HYDROCHLORIDE:Antiplatelet therapy:0.04), (CLOTRIMAZOLE:Antiplatelet therapy:1.31), (COAGULATION FACTOR IX HUMAN\COAGULATION FACTOR VII HUMAN\COAGULATION FACTOR X HUMAN\PROTEIN C\PROTEIN S HUMAN\PROTHROMBIN:Antiplatelet therapy:0.04), (CORTISONE\HYDROCORTISONE:Antiplatelet therapy:0.82), (DANAZOL:Antiplatelet therapy:0.6), (DAPSONE:Antiplatelet therapy:0.05), (DASATINIB:Antiplatelet therapy:1.39), (DESFLURANE:Antiplatelet therapy:0.64), (DEXTROSE\HEPARIN:Antiplatelet therapy:0.23), (DIMENHYDRINATE:Antiplatelet therapy:0.39), (DRONABINOL:Antiplatelet therapy:0.5), (DUTASTERIDE\TAMSULOSIN HYDROCHLORIDE:Antiplatelet therapy:0.25), (ECONAZOLE:Antiplatelet therapy:0.56), (ELETRIPTAN HYDROBROMIDE:Antiplatelet therapy:0.53), (ELOTUZUMAB:Antiplatelet therapy:0.01), (EMPAGLIFLOZIN\LINAGLIPTIN:Antiplatelet therapy:0.42), (EPINASTINE HYDROCHLORIDE:Antiplatelet therapy:0.02), (ERYTHROMYCIN:Antiplatelet therapy:0.48), (ESMOLOL HYDROCHLORIDE:Antiplatelet therapy:1), (ESTROGENS, CONJUGATED\MEDROXYPROGESTERONE ACETATE:Antiplatelet therapy:0.03), (ETORICOXIB:Antiplatelet therapy:0.47), (FENTANYL CITRATE:Antiplatelet therapy:0.1), (FINGOLIMOD HYDROCHLORIDE:Antiplatelet therapy:0.33), (FULVESTRANT:Antiplatelet therapy:0), (GEMFIBROZIL:Antiplatelet therapy:0.21), (GLIMEPIRIDE\ROSIGLITAZONE MALEATE:Antiplatelet therapy:0.4), (GLYCOPYRRONIUM\INDACATEROL:Antiplatelet therapy:0.87), (HYDROCHLOROTHIAZIDE\IRBESARTAN:Antiplatelet therapy:1.21), (HYDROXYCHLOROQUINE DIPHOSPHATE:Antiplatelet therapy:0.03), (HYOSCYAMINE:Antiplatelet therapy:0.02), (IDARUCIZUMAB:Antiplatelet therapy:1.52), (ILOPROST:Antiplatelet therapy:1.5), (IMIPRAMINE HYDROCHLORIDE:Antiplatelet therapy:0.3), (INDACATEROL:Antiplatelet therapy:0.12), (INDOMETHACIN:Antiplatelet therapy:0.35), (INSULIN BEEF:Antiplatelet therapy:0.05), (INSULIN GLULISINE:Antiplatelet therapy:0.54), (INTERFERON:Antiplatelet therapy:0.05), (INTERFERON ALFA-2A:Antiplatelet therapy:0.11), (IXAZOMIB:Antiplatelet therapy:0.17), (LACOSAMIDE:Antiplatelet therapy:0.22), (LEDIPASVIR\SOFOSBUVIR:Antiplatelet therapy:1.8), (LEUCOVORIN CALCIUM:Antiplatelet therapy:0.13), (LISDEXAMFETAMINE DIMESYLATE:Antiplatelet therapy:0.04), (LITHIUM CARBONATE:Antiplatelet therapy:1.05), (MAGNESIUM ASPARTATE HYDROCHLORIDE:Antiplatelet therapy:0.39), (MEGESTROL ACETATE:Antiplatelet therapy:0.15), (METFORMIN HYDROCHLORIDE\ROSIGLITAZONE MALEATE:Antiplatelet therapy:0.73), (METHENAMINE HIPPURATE:Antiplatelet therapy:0.34), (METHYLPHENIDATE HYDROCHLORIDE:Antiplatelet therapy:0.16), (METHYLPREDNISOLONE ACETATE:Antiplatelet therapy:0.41), (MIDODRINE:Antiplatelet therapy:0.68), (MODAFINIL:Antiplatelet therapy:0.38), (MUPIROCIN\MUPIROCIN CALCIUM:Antiplatelet therapy:0.11), (OSELTAMIVIR:Antiplatelet therapy:0.27), (PARICALCITOL:Antiplatelet therapy:0.76), (PATIROMER:Antiplatelet therapy:0.22), (PEGINTERFERON BETA-1A:Antiplatelet therapy:0.46), (PEMETREXED DISODIUM:Antiplatelet therapy:0.41), (PHENYTOIN SODIUM:Antiplatelet therapy:0.22), (PILSICAINIDE HYDROCHLORIDE:Antiplatelet therapy:0.32), (PIMOBENDAN:Antiplatelet therapy:0.01), (PIROXICAM:Antiplatelet therapy:0.25), (POSACONAZOLE:Antiplatelet therapy:0.38), (PRAZOSIN HYDROCHLORIDE:Antiplatelet therapy:0.03), (PREDNISOLONE\PREDNISOLONE ACETATE\PREDNISOLONE SODIUM SUCCINATE:Antiplatelet therapy:0.82), (PROPAFENONE HYDROCHLORIDE:Antiplatelet therapy:0.75), (QUINAPRIL\QUINAPRIL HYDROCHLORIDE:Antiplatelet therapy:0.1), (RIZATRIPTAN BENZOATE:Antiplatelet therapy:0.2), (ROPINIROLE:Antiplatelet therapy:0.35), (ROSIGLITAZONE MALEATE:Antiplatelet therapy:0.81), (SELEXIPAG:Antiplatelet therapy:0.54), (SODIUM PHOSPHATE, DIBASIC\SODIUM PHOSPHATE, MONOBASIC:Antiplatelet therapy:0.06), (SOLIFENACIN SUCCINATE:Antiplatelet therapy:0.47), (SOTALOL HYDROCHLORIDE:Antiplatelet therapy:0.24), (STREPTOKINASE:Antiplatelet therapy:0.11), (SUGAMMADEX:Antiplatelet therapy:0.06), (SULFAMETHOXAZOLE:Antiplatelet therapy:0.48), (SUMATRIPTAN:Antiplatelet therapy:0.25), (SUMATRIPTAN\SUMATRIPTAN SUCCINATE:Antiplatelet therapy:0.21), (SUNITINIB MALATE:Antiplatelet therapy:0.29), (TERAZOSIN HYDROCHLORIDE:Antiplatelet therapy:0.2), (TERBINAFINE HYDROCHLORIDE:Antiplatelet therapy:0.03), (TERBUTALINE:Antiplatelet therapy:0.36), (TESTOSTERONE CYPIONATE:Antiplatelet therapy:0.62), (TESTOSTERONE UNDECANOATE:Antiplatelet therapy:0.05), (TETRACYCLINE:Antiplatelet therapy:0.32), (TINZAPARIN:Antiplatelet therapy:0.14), (TOCILIZUMAB:Antiplatelet therapy:0.3), (UMECLIDINIUM BROMIDE\VILANTEROL TRIFENATATE:Antiplatelet therapy:0.4), (VASOPRESSIN:Antiplatelet therapy:0.29), (VECURONIUM BROMIDE:Antiplatelet therapy:0.55), (VORICONAZOLE:Antiplatelet therapy:0.09), (ZINC:Antiplatelet therapy:0.15), (ZONISAMIDE:Antiplatelet therapy:0.09), (THALIDOMIDE:Antipyresis:0.08), (ATAZANAVIR SULFATE\RITONAVIR:Antiretroviral therapy:0.16), (ATOVAQUONE:Antiretroviral therapy:0.35), (BISOPROLOL FUMARATE:Antiretroviral therapy:0.29), (BUMETANIDE:Antiretroviral therapy:0.03), (CERTOLIZUMAB PEGOL:Antiretroviral therapy:0.05), (CLINDAMYCIN\CLINDAMYCIN PHOSPHATE:Antiretroviral therapy:0.02), (CLOZAPINE:Antiretroviral therapy:0.13), (COBICISTAT:Antiretroviral therapy:0.16), (DOCUSATE SODIUM:Antiretroviral therapy:0.09), (ELVITEGRAVIR:Antiretroviral therapy:0.34), (EMTRICITABINE\RILPIVIRINE HYDROCHLORIDE\TENOFOVIR ALAFENAMIDE FUMARATE:Antiretroviral therapy:0.28), (ITRACONAZOLE:Antiretroviral therapy:0.2), (LITHIUM CARBONATE:Antiretroviral therapy:0.08), (LOVASTATIN:Antiretroviral therapy:0.15), (NAPROXEN:Antiretroviral therapy:0.41), (NEBIVOLOL:Antiretroviral therapy:0.05), (OMEPRAZOLE MAGNESIUM:Antiretroviral therapy:0.37), (OXCARBAZEPINE:Antiretroviral therapy:0.03), (PAROXETINE:Antiretroviral therapy:0.15), (PAROXETINE\PAROXETINE HYDROCHLORIDE:Antiretroviral therapy:0.07), (STREPTOMYCIN SULFATE:Antiretroviral therapy:0.18), (ZOLPIDEM\ZOLPIDEM TARTRATE:Antiretroviral therapy:0.05), (VINCRISTINE SULFATE:Antitussive therapy:0.01), (ACEBUTOLOL HYDROCHLORIDE:Antiviral prophylaxis:0.58), (ACETAMINOPHEN\CAFFEINE:Antiviral prophylaxis:0.1), (ACETAMINOPHEN\DIPHENHYDRAMINE:Antiviral prophylaxis:0.01), (ACETAMINOPHEN\PROPOXYPHENE NAPSYLATE:Antiviral prophylaxis:0.38), (ACLARUBICIN HYDROCHLORIDE:Antiviral prophylaxis:1.24), (ACTIVATED CHARCOAL:Antiviral prophylaxis:0.58), (ADO-TRASTUZUMAB EMTANSINE:Antiviral prophylaxis:0.87), (AGALSIDASE BETA:Antiviral prophylaxis:0.98), (ALCOHOL:Antiviral prophylaxis:0.59), (ALISKIREN HEMIFUMARATE:Antiviral prophylaxis:0.65), (AMINO ACIDS\CARBOHYDRATES\MINERALS\SOYBEAN OIL\VITAMINS:Antiviral prophylaxis:0.13), (AMISULPRIDE:Antiviral prophylaxis:0.88), (AMLODIPINE BESYLATE\HYDROCHLOROTHIAZIDE\VALSARTAN:Antiviral prophylaxis:0.21), (AMOXAPINE:Antiviral prophylaxis:0.03), (AMPICILLIN SODIUM:Antiviral prophylaxis:0.05), (ANTIHISTAMINES NOS:Antiviral prophylaxis:0.23),(ARMODAFINIL:Antiviral prophylaxis:1.13), (ASPARAGINASE ERWINIA CHRYSANTHEMI:Antiviral prophylaxis:0.39), (ASPIRIN\CLOPIDOGREL:Antiviral prophylaxis:0.32), (ASPIRIN\MAGNESIUM OXIDE:Antiviral prophylaxis:0.51), (ASUNAPREVIR:Antiviral prophylaxis:0.5), (ATAZANAVIR:Antiviral prophylaxis:0.55), (AXITINIB:Antiviral prophylaxis:0.58), (AZILSARTAN KAMEDOXOMIL:Antiviral prophylaxis:1.47), (AZITHROMYCIN OR METHOTREXATE SODIUM:Antiviral prophylaxis:0.42), (BENIDIPINE HYDROCHLORIDE:Antiviral prophylaxis:0.1), (BETAHISTINE MESILATE:Antiviral prophylaxis:0.01), (BETAMETHASONE DIPROPIONATE\CALCIPOTRIENE\CALCIPOTRIENE HYDRATE:Antiviral prophylaxis:0.06), (BILASTINE:Antiviral prophylaxis:0.58), (BOTULINUM TOXIN TYPE A:Antiviral prophylaxis:0.02), (BRIMONIDINE TARTRATE\TIMOLOL MALEATE:Antiviral prophylaxis:0.41), (BUCILLAMINE:Antiviral prophylaxis:0.33), (CABAZITAXEL:Antiviral prophylaxis:0.33), (CALCITONIN:Antiviral prophylaxis:0.81), (CALCIUM CARBONATE\CHOLECALCIFEROL\MAGNESIUM CARBONATE:Antiviral prophylaxis:0.28), (CALCIUM CARBONATE\ERGOCALCIFEROL\RETINOL:Antiviral prophylaxis:0.85), (CARISOPRODOL:Antiviral prophylaxis:0.78), (CARVEDILOL PHOSPHATE:Antiviral prophylaxis:1.36), (CEFOPERAZONE\SULBACTAM:Antiviral prophylaxis:0.04), (CEFPROZIL:Antiviral prophylaxis:0.33), (CHLORPHENIRAMINE\HYDROCODONE:Antiviral prophylaxis:0.5), (CHONDROITIN SULFATE (CHICKEN):Antiviral prophylaxis:0.18), (CLOMIPRAMINE HYDROCHLORIDE:Antiviral prophylaxis:0.64), (COBICISTAT\ELVITEGRAVIR\EMTRICITABINE\TENOFOVIR ALAFENAMIDE FUMARATE:Antiviral prophylaxis:0.39), (COBICISTAT\ELVITEGRAVIR\EMTRICITABINE\TENOFOVIR DISOPROXIL FUMARATE:Antiviral prophylaxis:0.28), (CORTICOTROPIN:Antiviral prophylaxis:0.65), (CYAMEMAZINE:Antiviral prophylaxis:0.21), (CYCLOPHOSPHAMIDE\CYTARABINE\DEXAMETHASONE\DOXORU BICIN\METHOTREXATE\VINCRISTINE:Antiviral prophylaxis:0.7), (DABRAFENIB:Antiviral prophylaxis:0.58), (DANTROLENE SODIUM:Antiviral prophylaxis:0.29), (DAPAGLIFLOZIN PROPANEDIOL:Antiviral prophylaxis:0.69), (DEFERIPRONE:Antiviral prophylaxis:0.35), (DEFLAZACORT:Antiviral prophylaxis:1.5), (DEGARELIX:Antiviral prophylaxis:0.19), (DESMOPRESSIN:Antiviral prophylaxis:0.05), (DESONIDE:Antiviral prophylaxis:0.75), (DESVENLAFAXINE SUCCINATE:Antiviral prophylaxis:0.71), (DEXTROAMPHETAMINE SULFATE:Antiviral prophylaxis:0.03), (DIGITOXIN:Antiviral prophylaxis:0.87), (DOLASETRON MESYLATE:Antiviral prophylaxis:0.17), (DORNASE ALFA:Antiviral prophylaxis:0.23), (DORZOLAMIDE HYDROCHLORIDE:Antiviral prophylaxis:0.4), (DOXYLAMINE SUCCINATE:Antiviral prophylaxis:0.31), (DRONEDARONE:Antiviral prophylaxis:0.64), (DUTASTERIDE\TAMSULOSIN HYDROCHLORIDE:Antiviral prophylaxis:0.24), (ECONAZOLE:Antiviral prophylaxis:2), (ELDECALCITOL:Antiviral prophylaxis:1.29), (ELTROMBOPAG OLAMINE:Antiviral prophylaxis:0.39), (EMPAGLIFLOZIN\LINAGLIPTIN:Antiviral prophylaxis:0.75), (EMTRICITABINE\TENOFOVIR:Antiviral prophylaxis:0.13), (ESTAZOLAM:Antiviral prophylaxis:0.02), (ESTROGENS, CONJUGATED\MEDROXYPROGESTERONE ACETATE:Antiviral prophylaxis:0.41), (ESTRONE\SUS SCROFA OVARY:Antiviral prophylaxis:0.26), (ETHACRYNIC ACID:Antiviral prophylaxis:0.06), (ETHINYL ESTRADIOL\NORGESTIMATE:Antiviral prophylaxis:0.16), (ETOPOSIDE PHOSPHATE:Antiviral prophylaxis:1.34), (ETRETINATE:Antiviral prophylaxis:0.73), (EVOLOCUMAB:Antiviral prophylaxis:1.51), (FAROPENEM SODIUM:Antiviral prophylaxis:0.02), (FENOFIBRIC ACID:Antiviral prophylaxis:0.45), (FESOTERODINE FUMARATE:Antiviral prophylaxis:1.59), (FEXOFENADINE\PSEUDOEPHEDRINE:Antiviral prophylaxis:0.2), (FLUOCINOLONE ACETONIDE:Antiviral prophylaxis:0.53), (FLUOROMETHOLONE:Antiviral prophylaxis:0.18), (FLUTICASONE PROPIONATE\FORMOTEROL FUMARATE:Antiviral prophylaxis:0.06), (FLUVOXAMINE MALEATE:Antiviral prophylaxis:0.65), (FONDAPARINUX:Antiviral prophylaxis:0.13), (FORMOTEROL FUMARATE:Antiviral prophylaxis:1.45), (FORMOTEROL FUMARATE\MOMETASONE FUROATE:Antiviral prophylaxis:0.92), (FOSPHENYTOIN\FOSPHENYTOIN SODIUM:Antiviral prophylaxis:0.45), (FULVESTRANT:Antiviral prophylaxis:0.47), (GARENOXACIN:Antiviral prophylaxis:0.76), (GIMERACIL\OTERACIL\TEGAFUR:Antiviral prophylaxis:0.54), (GLYCOPYRROLATE\INDACATEROL MALEATE:Antiviral prophylaxis:0.21), (GLYCOPYRRONIUM:Antiviral prophylaxis:0.74), (GUSPERIMUS TRIHYDROCHLORIDE:Antiviral prophylaxis:2), (HUMAN BLOOD CELLS:Antiviral prophylaxis:0.06), (HYDROCHLOROTHIAZIDE\TELMISARTAN:Antiviral prophylaxis:1.28), (HYDROCORTISONE HEMISUCCINATE:Antiviral prophylaxis:0.21), (HYDROXYCHLOROQUINE DIPHOSPHATE:Antiviral prophylaxis:0.37), (HYOSCYAMINE:Antiviral prophylaxis:0.84), (IBRITUMOMAB TIUXETAN:Antiviral prophylaxis:2.21), (IDARUBICIN HYDROCHLORIDE:Antiviral prophylaxis:1.34), (IMIGLUCERASE:Antiviral prophylaxis:0.44), (IMIPRAMINE HYDROCHLORIDE:Antiviral prophylaxis:0.4), (INFLIXIMAB-DYYB:Antiviral prophylaxis:0.01), (INTERFERON ALFA-2A:Antiviral prophylaxis:0.98), (INTERFERON ALFA-2B:Antiviral prophylaxis:0.59), (INTERFERON GAMMA-1B:Antiviral prophylaxis:0.96), (IODINE:Antiviral prophylaxis:0.12), (IOPAMIDOL:Antiviral prophylaxis:0.07), (ISAVUCONAZOLE:Antiviral prophylaxis:0.18), (LAFUTIDINE:Antiviral prophylaxis:0.52), (LANREOTIDE ACETATE:Antiviral prophylaxis:0.26), (LEVOCARNITINE HYDROCHLORIDE:Antiviral prophylaxis:0.04), (LEVOLEUCOVORIN CALCIUM:Antiviral prophylaxis:0.56), (LEVOTHYROXINE SODIUM\POTASSIUM IODIDE:Antiviral prophylaxis:0.39), (LOMITAPIDE MESYLATE:Antiviral prophylaxis:0.15), (LYMPHOCYTES:Antiviral prophylaxis:0.37), (MAGNESIUM ASPARTATE HYDROCHLORIDE:Antiviral prophylaxis:0.89), (MAGNESIUM ASPARTATE\POTASSIUM ASPARTATE:Antiviral prophylaxis:0.36), (MECLIZINE\MECLIZINE HYDROCHLORIDE:Antiviral prophylaxis:0.37), (MEDROXYPROGESTERONE:Antiviral prophylaxis:0.1), (MEFENAMIC ACID:Antiviral prophylaxis:0.14), (MELPERONE:Antiviral prophylaxis:0.05), (METFORMIN HYDROCHLORIDE\VILDAGLIPTIN:Antiviral prophylaxis:0.56), (METFORMIN PAMOATE:Antiviral prophylaxis:0.19), (METHENAMINE HIPPURATE:Antiviral prophylaxis:1.33), (METHOTREXATE\METHOTREXATE SODIUM:Antiviral prophylaxis:0.19), (MEXILETINE HYDROCHLORIDE:Antiviral prophylaxis:0.04), (MIGLUSTAT:Antiviral prophylaxis:0.48), (MIZORIBINE:Antiviral prophylaxis:1.27), (MOEXIPRIL HYDROCHLORIDE:Antiviral prophylaxis:0.01), (NATEGLINIDE:Antiviral prophylaxis:0.96), (NIRAPARIB:Antiviral prophylaxis:0.35), (NORTRIPTYLINE:Antiviral prophylaxis:1.25), (OMEGA-3-ACID ETHYL ESTERS:Antiviral prophylaxis:1.53), (PANTOPRAZOLE MAGNESIUM:Antiviral prophylaxis:1.85), (PARATHYROID HORMONE:Antiviral prophylaxis:0.24), (PARICALCITOL:Antiviral prophylaxis:0.74), (PATIROMER:Antiviral prophylaxis:0.29), (PAZOPANIB HYDROCHLORIDE:Antiviral prophylaxis:1.6), (PAZUFLOXACIN:Antiviral prophylaxis:0.28), (PEGINTERFERON ALFA-2A OR PEGINTERFERON ALFA-2B:Antiviral prophylaxis:0.05), (PEGINTERFERON BETA-1A:Antiviral prophylaxis:1.05), (PEMETREXED:Antiviral prophylaxis:1.04), (PEMETREXED DISODIUM:Antiviral prophylaxis:1.5), (PENICILLIN G:Antiviral prophylaxis:0.84), (PHOSPHOLIPID:Antiviral prophylaxis:0.45), (PILSICAINIDE HYDROCHLORIDE:Antiviral prophylaxis:0.07), (PIMOBENDAN:Antiviral prophylaxis:0.44), (PIRITRAMIDE:Antiviral prophylaxis:0.31), (POLYMYXIN:Antiviral prophylaxis:0.26), (PORFIMER SODIUM:Antiviral prophylaxis:0.26), (PREDNISOLONE SODIUM PHOSPHATE:Antiviral prophylaxis:1.77), (PYRIMETHAMINE:Antiviral prophylaxis:0.19), (QUINACRINE:Antiviral prophylaxis:0.21), (QUINAPRIL\QUINAPRIL HYDROCHLORIDE:Antiviral prophylaxis:0.05), (RAMUCIRUMAB:Antiviral prophylaxis:0.23), (RANIMUSTINE:Antiviral prophylaxis:0.63), (REGORAFENIB:Antiviral prophylaxis:0.71), (RETINOL:Antiviral prophylaxis:1.04), (RIZATRIPTAN:Antiviral prophylaxis:0.27), (ROFLUMILAST:Antiviral prophylaxis:1.48), (SACUBITRIL\VALSARTAN:Antiviral prophylaxis:1.01), (SALMETEROL:Antiviral prophylaxis:0.94), (SALMETEROL XINAFOATE:Antiviral prophylaxis:0.35), (SARPOGRELATE:Antiviral prophylaxis:0.59), (SECUKINUMAB:Antiviral prophylaxis:1.5), (SIPULEUCEL-T:Antiviral prophylaxis:0.06), (SODIUM CITRATE:Antiviral prophylaxis:0.27), (SODIUM FERROUS CITRATE:Antiviral prophylaxis:0.83), (SODIUM OXYBATE:Antiviral prophylaxis:0.97), (SOTALOL HYDROCHLORIDE:Antiviral prophylaxis:0.07), (SUFENTANIL CITRATE:Antiviral prophylaxis:0.26), (SULBACTAM SODIUM:Antiviral prophylaxis:0.35), (SUMATRIPTAN\SUMATRIPTAN SUCCINATE:Antiviral prophylaxis:1.47), (SUPLATAST TOSILATE:Antiviral prophylaxis:0.09), (TAPENTADOL HYDROCHLORIDE:Antiviral prophylaxis:0.1), (TEDUGLUTIDE\WATER:Antiviral prophylaxis:0.21), (THEOPHYLLINE:Antiviral prophylaxis:0.12), (THROMBOPOIETIN:Antiviral prophylaxis:0.81), (THYROID, PORCINE:Antiviral prophylaxis:0.14), (TRAMETINIB:Antiviral prophylaxis:0.44), (TRANDOLAPRIL:Antiviral prophylaxis:0.29), (TRIHEXYPHENIDYL:Antiviral prophylaxis:0.07), (TRIMIPRAMINE MALEATE:Antiviral prophylaxis:0.36), (UMECLIDINIUM BROMIDE:Antiviral prophylaxis:0.43), (VEMURAFENIB:Antiviral prophylaxis:0.39), (VILAZODONE HYDROCHLORIDE:Antiviral prophylaxis:0.38), (VINDESINE SULFATE:Antiviral prophylaxis:1.21), (VINORELBINE\VINORELBINE TARTRATE:Antiviral prophylaxis:0.24), (ZAFIRLUKAST:Antiviral prophylaxis:0.26), (ZILEUTON:Antiviral prophylaxis:0.28), (ZINC:Antiviral prophylaxis:1.07), (ZOLMITRIPTAN:Antiviral prophylaxis:0.12), (ACETAMINOPHEN\BUTALBITAL\CAFFEINE:Antiviral treatment:0.29), (ADO-TRASTUZUMAB EMTANSINE:Antiviral treatment:0.01), (ALBUTEROL SULFATE\IPRATROPIUM BROMIDE:Antiviral treatment:0.3), (ALCOHOL:Antiviral treatment:0.41), (ALENDRONIC ACID:Antiviral treatment:0.16), (ALFACALCIDOL:Antiviral treatment:0.1), (AMPHETAMINE ASPARTATE\AMPHETAMINE SULFATE\DEXTROAMPHETAMINE SACCHARATE\DEXTROAMPHETAMINE SULFATE:Antiviral treatment:0.36),(ARMODAFINIL:Antiviral treatment:0.24), (ASPARAGINASE:Antiviral treatment:0.46), (BELATACEPT:Antiviral treatment:0.38), (BLINATUMOMAB:Antiviral treatment:1.01), (BUMETANIDE:Antiviral treatment:0.69), (BUSPIRONE HYDROCHLORIDE:Antiviral treatment:0.25), (CALCIUM ACETATE:Antiviral treatment:0.53), (CANDESARTAN CILEXETIL\HYDROCHLOROTHIAZIDE:Antiviral treatment:0.63), (CEFTRIAXONE SODIUM:Antiviral treatment:1), (CEFUROXIME:Antiviral treatment:0.12), (CHOLESTYRAMINE:Antiviral treatment:0.87), (CLOZAPINE:Antiviral treatment:0.39), (CORTISONE\HYDROCORTISONE:Antiviral treatment:0.17), (DACLIZUMAB:Antiviral treatment:0.82), (DALTEPARIN:Antiviral treatment:0.28), (DANAZOL:Antiviral treatment:0.09), (DARATUMUMAB:Antiviral treatment:0.11), (DARUNAVIR\RITONAVIR:Antiviral treatment:0.25), (DESLORATADINE:Antiviral treatment:0.32), (DIVALPROEX SODIUM:Antiviral treatment:1.05), (DULOXETINE:Antiviral treatment:0.41), (ELETRIPTAN HYDROBROMIDE:Antiviral treatment:0.01), (ESZOPICLONE:Antiviral treatment:0.95), (EVOLOCUMAB:Antiviral treatment:0.82), (EZETIMIBE\SIMVASTATIN:Antiviral treatment:0.02), (FESOTERODINE FUMARATE:Antiviral treatment:0.05), (FLUTICASONE FUROATE\SALMETEROL XINAFOATE:Antiviral treatment:0.11), (FLUTICASONE\FLUTICASONE PROPIONATE:Antiviral treatment:0.12), (FOSAMPRENAVIR:Antiviral treatment:0.04), (FULVESTRANT:Antiviral treatment:0.03), (HYDROCORTISONE SODIUM SUCCINATE:Antiviral treatment:0.04), (HYDROXYCHLOROQUINE DIPHOSPHATE:Antiviral treatment:0.02), (HYDROXYZINE PAMOATE:Antiviral treatment:0.2), (IBRITUMOMAB TIUXETAN:Antiviral treatment:0.95), (IMATINIB MESYLATE:Antiviral treatment:0.96), (IMIPRAMINE HYDROCHLORIDE:Antiviral treatment:0.07), (INDAPAMIDE:Antiviral treatment:0.03), (INDAPAMIDE\PERINDOPRIL:Antiviral treatment:0.16), (INTERFERON GAMMA-1B:Antiviral treatment:0.06), (IPRATROPIUM:Antiviral treatment:0.11), (ISOSORBIDE:Antiviral treatment:0.16), (LABETALOL\LABETALOL HYDROCHLORIDE:Antiviral treatment:0.4), (LERCANIDIPINE:Antiviral treatment:1.06), (LERCANIDIPINE HYDROCHLORIDE:Antiviral treatment:0.2), (LINAGLIPTIN:Antiviral treatment:0.26), (LITHIUM CARBONATE:Antiviral treatment:0.15), (METAMIZOLE:Antiviral treatment:0.23), (METHYLPREDNISOLONE ACETATE:Antiviral treatment:0.41), (NELFINAVIR:Antiviral treatment:0.1), (OXCARBAZEPINE:Antiviral treatment:1.23), (PANTOPRAZOLE MAGNESIUM:Antiviral treatment:0.07), (PAROXETINE:Antiviral treatment:0.39), (PAZOPANIB HYDROCHLORIDE:Antiviral treatment:0.26), (PERINDOPRIL:Antiviral treatment:0.21), (PHENYTOIN:Antiviral treatment:0.39), (PREDNISOLONE SODIUM PHOSPHATE:Antiviral treatment:0.43), (PRIMIDONE:Antiviral treatment:0.02), (RANOLAZINE:Antiviral treatment:0.37), (SACUBITRIL\VALSARTAN:Antiviral treatment:0.56), (SECUKINUMAB:Antiviral treatment:0.81), (SODIUM OXYBATE:Antiviral treatment:0.17), (STREPTOMYCIN\STREPTOMYCIN SULFATE:Antiviral treatment:0.36), (SUMATRIPTAN\SUMATRIPTAN SUCCINATE:Antiviral treatment:0.12), (TELMISARTAN:Antiviral treatment:0.59), (TETRACYCLINE:Antiviral treatment:0.66), (TIMOLOL:Antiviral treatment:0.17), (TIOTROPIUM:Antiviral treatment:0.01), (TIZANIDINE:Antiviral treatment:0.13), (TOCILIZUMAB:Antiviral treatment:0.32), (TOFACITINIB CITRATE:Antiviral treatment:0.09), (VARENICLINE TARTRATE:Antiviral treatment:0.2), (VERAPAMIL HYDROCHLORIDE:Antiviral treatment:0.91), (ZONISAMIDE:Antiviral treatment:0.05), (.ALPHA.-TOCOPHEROL ACETATE, D-\ASCORBIC ACID\CALCIUM PANTOTHENATE\FOLIC ACID\MANGANESE\NIACINAMIDE\PYRIDOXINE\RIBOFLAVIN\THIAMINE\VITAMIN A\ZINC OXIDE:Anxiety:0.51), (.ALPHA.-TOCOPHEROL\ASCORBIC ACID\\BIOTIN\CALCIUM CARBONATE\CHOLECALCIFEROL\COPPER\CYANOCOBALAMIN\FERROUS FUMARATE\FOLIC ACID\IODINE\MAGNESIUM\NIACINAMIDE\PANTOTHENIC ACID\PYRIDOXINE\RIBOFLAVIN\THIAMINE\VITAMIN A\ZINC:Anxiety:0.07), (.BETA.-ACETYLDIGOXIN:Anxiety:0.37), (.BETA.-HYDROXYISOVALERIC ACID\ARGININE\GLUTAMINE:Anxiety:0.54), (2-HYDROXYETHYL METHACRYLATE:Anxiety:0.19), (ACETAMINOPHEN\ASPIRIN:Anxiety:0.43), (ACETAMI NOPHEN\ASPIRIN\CAFFEINE\CODEINE PHOSPHATE:Anxiety:0.1), (ACETAMINOPHEN\BUTYLSCOPOLAMINE BROMIDE:Anxiety:0.17), (ACETAMINOPHEN\CAFFEINE\CODEINE PHOSPHATE:Anxiety:0.05), (ACETAMINOPHEN\CAFFEINE\PROPOXYPHENE:Anxiety:0.73), (ACETAMINOPHEN\CHLORPHENIRAMINE MALEATE\DEXTROMETHORPHAN HYDROBROMIDE\PSEUDOEPHEDRINE HYDROCHLORIDE:Anxiety:0.44), (ACETAMINOPHEN\DIPHENHYDRAMINE\DIPHENH YDRAMINE CITRATE:Anxiety:0.31), (ACETAMINOPHEN\PHENYLEPHRINE:Anxiety:0.08), (ACETIC ACID\SODIUM ACETATE:Anxiety:0.2), (ACETOHYDROXAMIC ACID:Anxiety:0.4), (ACETYLCYSTEINE\BENZALKONIUM CHLORIDE\TUAMINOHEPTANE SULFATE:Anxiety:1.01), (ACRIVASTINE:Anxiety:0.14), (ADENINE:Anxiety:0.13), (ALBUTE ROL\GUAIFENESIN:Anxiety:0.74), (ALBUTEROL\IPRATROPIUM BROMIDE:Anxiety:0.95), (ALDESLEUKIN:Anxiety:1.34), (ALISKIREN HEMIFUMARATE\HYDROCHLOROTHIAZIDE:Anxiety:0.96), (ALISKIREN HEMIFUMARATE\VALSARTAN:Anxiety:0.43), (ALISKIREN\HYDROCHLOROTHIAZIDE:Anxiety:0.42), (ALLANTOIN\BENZOCAINE\CAMPHOR (NATURAL)\DIMETHICONE\MENTHOL:Anxiety:0.69), (ALPROSTADIL\PAPAVERINE\P HENTOLAMINE:Anxiety:0.31), (ALUMINUM HYDROXIDE\MAGNESIUM OXIDE\OXETHAZAINE:Anxiety:1.23), (AMILORIDE HYDROCHLORIDE\FUROSEMIDE:Anxiety:1.27), (AMINO ACIDS\CARBOHYDRATES\MINERALS\SOYBEAN OIL\VITAMINS:Anxiety:0.26), (AMINOBENZOIC ACID\CALCIUM PANTOTHENATE\CYSTINE\KERATIN\THIAMINE MONONITRATE:Anxiety:1.01), (AMINOPHYLLINE\QUININE SULFATE:Anxiety:0.26), (AMLODIPINE BESYLATE\ATORVASTATIN CALCIUM TRIHYDRATE:Anxiety:0.13), (AMLODIPINE BESYLATE\IRBESARTAN:Anxiety:0.55), (AMLODIPINE MALEATE:Anxiety:0.65), (AMMONIUM GLYCYRRHIZATE\GLYCINE\METHIONINE:Anxiety:0.03), (AMOROLFINE:Anxiety:0.16), (AMPHETAMINE ASPARTATE MONOHYDRATE\AMPHETAMINE SULFATE\DEXTROAMPHETAMINE SACCHARATE\DEXTROAMPHETAMINE SULFATE:Anxiety:0.54), (AMPHETAMINE ASPARTATE\DEXTROAMPHETAMINE SACCHARATE:Anxiety:0.69), (AMPICILLIN SODIUM:Anxiety:0.51), (ANHYDROUS CITRIC ACID\MAGNESIUM OXIDE\SODIUM PICOSULFATE:Anxiety:0.6), (ANIDULAFUNGIN:Anxiety:1.14), (APOMORPHINE:Anxiety:00.66), (ARCTOSTAPHYLOS UVA-URSI LEAF\URTICA DIOICA:Anxiety:0.73), (ARGATROBAN:Anxiety:0.03), (ARNICA MONTANA:Anxiety:0.02), (ARTEMETHER\LUMEFANTRINE:Anxiety:0.14), (ARTICAINE HYDROCHLORIDE\EPINEPHRINE BITARTRATE:Anxiety:0.09), (ASCORBIC ACID\BIOTIN\CALCIUM PANTOTHENATE\CYANOCOBALAMIN\FOLIC ACID\NIACINAMIDE\PYRIDOXINE HYDROCHLORIDE\RIBOFLAVIN\THIAMINE HYDROCHLORIDE:Anxiety:0.49), (ASCORBIC ACID\IRON:Anxiety:0.99), (ASPARTAME:Anxiety:0.12), (ASPIRIN\CAFFEINE\SALICYLA MIDE:Anxiety:1.13), (ATOVAQUONE\PROGUANIL:Anxiety:0.3), (ATROPA BELLADONNA:Anxiety:0.13), (ATROPINE\DIPHENOXYLATE HYDROCHLORIDE:Anxiety:0.07), (AVOBENZONE\HOMOSALATE\OCTISALATE:Anxiety: 0.22), (BACILLUS CALMETTE-GUERIN SUBSTRAIN CONNAUGHT LIVE ANTIGEN:Anxiety:0.01), (BACITRACIN ZINC:Anxiety:1.08), (BACITRACIN\NEOMYCIN SULFATE\POLYMYXIN B SULFATE:Anxiety:0.12), (BACLOFEN\CYCLOBENZAPRINE\DICLOFENAC\GABAPENTI N\TETRACAINE:Anxiety:0.65), (BALSAM PERU\BISMUTH HYDROXIDE\BISMUTH SUBGALLATE\BORIC ACID\ZINC OXIDE:Anxiety:0.15), (BENPERIDOL:Anxiety:0.22), (BENZALKONIUM CHLORIDE:Anxiety:0.17), (BENZOCAINE\RESORCINOL:Anxiety:0.89), (BEPOTASTINE\B EPOTASTINE BESILATE:Anxiety:0.19), (BEPRIDIL:Anxiety:0.72), (BETA GLUCAN:Anxiety:0.57), (BETAINE:Anxiety:0.48), (BETAMETHASONE ACETATE\BETAMETHASONE SODIUM PHOSPHATE:Anxiety:0.71), (BETAMETHASONE DIPROPIONATE\CALCIPOTRIENE HYDRATE:Anxiety:1.03), (BETAMETHASONE DIPROPIONATE\CLOTRIMAZOLE\GENTAMICIN SULFATE:Anxiety:0.06), (BEXAROTENE:Anxiety:0.26), (BORIC ACID:Anxiety:1.05), (BORNAPRINE HYDROCHLORIDE:Anxiety:0.21), (BORON:Anxiety:0.8), (BOTULINUM TOXIN TYPE A:Anxiety:2.5), (BRINZOLAMIDE\TIMOLOL MALEATE:Anxiety:0.96), (BUDESONIDE\INDACATEROL MALEATE:Anxiety:0.17), (CAFFEINE\ERGOTAMINE TARTRATE:Anxiety:0.52), (CAFFEINE\ISOMETHEPTENE\METHIMAZOLE:Anxiety:0.7), (CAJUPUT OIL\EUCALYPTUS OIL\PEPPERMINT OIL:Anxiety:1.1), (CALCIUM ACETATE\MAGNESIUM ACETATE\POTASSIUM ACETATE\SODIUM ACETATE\SODIUM CHLORIDE:Anxiety:0.72), (CALCIUM BICARBONATE:Anxiety:0.57), (CALCIUM CARBIMIDE:Anxiety:0.59), (CALCIUM CARBONATE\ETIDRONATE DISODIUM:Anxiety:1.31), (CALCIUM PANTOTHENATE\NIACINAMIDE\PYRIDOXINE HYDROCHLORIDE\RIBOFLAVIN\THIAMINE HYDROCHLORIDE:Anxiety:0.03), (CALCIUM PHOSPHATE:Anxiety:0.38), (CAMPHOR (SYNTHETIC)\LEVOMENTHOL\MENTHYL SALICYLATE OR CAMPHOR (SYNTHETIC)\MENTHOL\MENTHYL SALICYLATE OR MENTHOL\MENTHYL SALICYLATE:Anxiety:0.35), (CANNABIS SATIVA SEED OIL:Anxiety:0.36), (CAPSICUM:Anxiety:0.66), (CARBASPIRIN:Anxiety:0.5), (CARBOPROST TROMETHAMINE:Anxiety:0.08), (CARBOXYMETHYLCELLULOSE SODIUM\HYPROMELLOSES:Anxiety:0.21), (CARBOXYMETHYLCELLULOSE SODIUM\SORBINICATE:Anxiety:0.5), (CARMUSTINE:Anxiety:0.69), (CARTEOLOL HYDROCHLORIDE:Anxiety:0.65), (CASTOR OIL:Anxiety:0.14), (CAT'S CLAW:Anxiety:0.07), (CEFOPERAZONE\SULBACTAM:Anxiety:0.02), (CEFOTAXIME\CEF OTAXIME SODIUM:Anxiety:2.1), (CELERY SEED:Anxiety:0.35), (CEPHALOSPORIN C:Anxiety:0.4), (CEPHRADINE:Anxiety:0.45), (CERIVASTATIN SODIUM:Anxiety:1.33), (CHLOPHEDIANOL HYDROCHLORIDE\DEXBROMPHENIRAMINE MALEATE\PHENYLEPHRINE HYDROCHLORIDE:Anxiety:0.52), (CHLORMADINONE ACETATE\ETHINYL ESTRADIOL:Anxiety:0.9), (CHLOROCRESOL:Anxiety:0.08), (CHLOROQUINE PHOSPHATE:Anxiety:0.5), (CHLORPHENIRAMINE MALEATE\HYDROCODONE BITARTRATE\PSEUDOEPHEDRINE HYDROCHLORIDE:Anxiety:0.11), (CHLORPHENIRAMINE MALEATE\PHENYLEPHRINE HYDROCHLORIDE:Anxiety:0.36), (CHOLINE BITARTRATE:Anxiety:0.14), (CHOLINE\TRAZODONE HYDROCHLORIDE:Anxiety:0.39), (CICLOPIROX OLAMINE:Anxiety:1.46), (CIMETIDINE\FAMOTIDINE\RANITIDINE HYDROCHLORIDE:Anxiety:0.11), (CINNAMON OIL:Anxiety:0.08), (CIPROFLOXACIN\DEXAMETHASONE:Anxiety:0.24), (CITICOLINE:Anxiety: 0.89), (CITRIC ACID MONOHYDRATE\POTASSIUM CITRATE:Anxiety:0.66), (CITRIC ACID MONOHYDRATE\TRISODIUM CITRATE DIHYDRATE:Anxiety:0.25), (CLADRIBINE:Anxiety:1.79), (CLIDINIUM BROMIDE:Anxiety:0.62), (CLONIXIN\CYCLOBENZAPRINE:Anxiety:0.31), (CLOPIDOGRE L BESILATE:Anxiety:0.01), (CLOPREDNOL:Anxiety:0.03), (CLOSTRIDIUM TETANI\CLOSTRIDIUM TETANI TOXOID ANTIGEN (FORMALDEHYDE INACTIVATED)\CORYNEBACTERIUM DIPHTHERIAE\CORYNEBACTERIUM DIPHTHERIAE TOXOID ANTIGEN (FORMALDEHYDE INACTIVATED):Anxiety:0.26), (COBALAMIN:Anxiety:1.41), (CODEINE PHOSPHATE\DICLOFENAC SODIUM:Anxiety:1.06), (CODEINE PHOSPHATE\GUAIFENESIN\PSEUDOEPHEDRINE HYDROCHLORIDE:Anxiety:0.42), (CODEINE PHOSPHATE\PHENYLEPHRINE HYDROCHLORIDE\PROMETHAZINE HYDROCHLORIDE:Anxiety:0.24), (COLISTIMETHATE SODIUM:Anxiety:2.09), (COPPER GLUCONATE:Anxiety:0.18), (COPPER\INTRAUTERINE DEVICE:Anxiety:0.03), (COUMARIN:Anxiety:0.55), (CREATININE:Anxiety:0.6), (CROTALI DAE POLYVALENT IMMUNE FAB (OVINE):Anxiety:0.48), (CUMIN:Anxiety:0), (CYANOCOBALAMIN\FOLIC ACID\IRON:Anxiety:0.39), (CYANOCOBALAMIN\PYRIDOXINE HYDROCHLORIDE\RIBOFLAVIN\SODIUM PANTOTHENATE\THIAMINE HYDROCHLORIDE\VITAMIN B COMPLEX:Anxiety:0.34), (CYANOCOBALAMIN\PYRIDOXINE HYDROCHLORIDE\THIAMINE DISULFIDE:Anxiety:1.13), (CYSTEINE:Anxiety:0.36), (DEMECLOCYCLINE:Anxiety:0.44), (DEOXYRIBONUCLIEC ACID:Anxiety:0.53), (DESLORATADINE\PSEUDOEPHEDRINE SULFATE:Anxiety:0.58), (DEUTETRABENAZINE:Anxiety:1.13), (DEXAMETHASONE\FRA MYCETIN SULFATE\GRAMICIDIN:Anxiety:0.42), (DEXAMETHASONE\NEOMYCIN\POLYMYXIN B SULFATE:Anxiety:0.06), (DEXTROMETHORPHAN HYDROBROMIDE\GUAIFENESIN\PHENYLEPHRINE HYDROCHLORIDE:Anxiety:0.17), (DEXTROMETHORPHAN HYDROBROMIDE\GUAIFENESIN\PSEUDOEPHEDRINE HYDROCHLORIDE:Anxiety:0.31), (DEXTROSE MONOHYDRATE\SODIUM CHLORIDE:Anxiety:0.13), (DICHLORPHENAMIDE:Anxiety:0.69), (DICLOFENAC DIETHYLAMINE:Anxiety:0.79), (DIDANOSINE:Anxiety:0.57), (DIETARY SUPPLEMENT\FLAVOCOXID:Anxiety:0.58), (DIETHYLPROPION:Anxiety:0.28), (DIETHY LSTILBESTROL:Anxiety:0.04), (DIGITALIS:Anxiety:0.65), (DIHYDRALAZINE SULFATE:Anxiety:0.39), (DIMETHYL SULFOXIDE:Anxiety:1.48), (DOCONEXENT\ICOSAPENT\PHOSPHATIDYL SERINE:Anxiety:0.26), (DROSPIRENONE\ESTRADIOL:Anxiety:1.12), (ECHINACEA PURPUREA:Anxiety:0.26), (EDOXABAN TOSILATE MONOHYDRATE:Anxiety:0.79), (EFLORNITHINE HYDROCHLORIDE:Anxiety:0.72), (ELECTROLYTES NOS\POLYETHYLENE GLYCOL 3350:Anxiety:0.53), (EMTRICITABINE\TENOFOVIR:Anxiety:0.21), (ENALAPRIL MALEATE\LERCANIDIPINE HYDROCHLORIDE:Anxiety:1.29), (EPHEDRA:Anxiety:0.14), (ERGOLOID MESYLATES:Anxiety:1.11), (ERYTHROMYCIN\SULFISOXAZOLE:Anxiety:0.9), (ESCHERI CHIA COLI:Anxiety:0.87), (ESTRADIOL HEMIHYDRATE\NORETHINDRONE ACETATE:Anxiety:0.27), (ESTRADIOL VALERATE:Anxiety:0.43), (ESTROGENS, CONJUGATED SYNTHETIC A:Anxiety:0.43), (ESTROGENS, CONJUGATED SYNTHETIC B:Anxiety:1), (ETEPLIRSEN:Anxiety:0.05), (ETHINYL ESTRADIOL\ETHYNODIOL:Anxiety:0.64), (ETHIODIZED OIL:Anxiety:0.32), (ETRETINATE:Anxiety:0.9), (EZETIMIBE\ROSUVASTATIN:Anxiety:0.18), (FECAL MICROBIOTA:Anxiety:0.05), (FERRIC HYDROXIDE:Anxiety:0.22), (FERROUS GLYCINE SULFATE:Anxiety:1.11), (FIBRINOGEN HUMAN:Anxiety:0.24), (FLAVOXATE HYDROCHLORIDE:Anxiety:0.45), (FLUCYTOSINE:Anxiety:1.11), (FLUNARIZINE HYDROCHLORIDE:Anxiety:1.61), (FLUOROURACIL\IRINOTECAN\LEUCOVORIN:Anxiety: 0.57), (FLUPENTIXOL DECANOATE:Anxiety:1.24), (FLUPENTIXOL DIHYDROCHLORIDE:Anxiety:0.93), (FLUPIRTINE:Anxiety:0.61), (FLURANDRENOLIDE: Anxiety:0.29), (FLUTAMIDE:Anxiety:0.93), (FOSCARNET:Anxiety:0.08), (FOSFOMYCIN TROMETHAMINE:Anxiety:0.1), (FRANGULA PURSHIANA BARK:Anxiety:0.44), (FUROSEMIDE\POTASSIUM CHLORIDE:Anxiety:0.32), (GADOBENATE DIMEGLUMINE:Anxiety:0.8), (GADOVERSETAMIDE:Anxiety:0.89), (GELATIN:Anxiety:0.55), (GENISTEIN:Anxiety:0.43), (GENTIAN VIOLET:Anxiety:0.42), (GILTERITINIB:Anxiety:0.03), (GIMERACIL\OTERACIL\TEGAFUR:Anxiety:1.74), (GINSENG EXTRACT:Anxiety:0.38), (GLUCONATE:Anxiety:0.3), (GOLD:Anxiety:0.01), (GOTU KOLA:Anxiety:0.52), (GRAPEFRUIT SEED EXTRACT:Anxiety:0.25), (HERBALS\VITIS VINIFERA SEED:Anxiety:0.27), (HEXACHLOROPHENE:Anxiety:0.12), (HIGH DENSITY POLYETHYLENE:Anxiety:1.29), (HUMAN COLOSTRUM:Anxiety:0.49), (HYDRALAZINE HYDROCHLORIDE\ISOSORBIDE DINITRATE:Anxiety:0.42), (HYDROCHLORIC ACID:Anxiety:0.53), (HYDROCHLOROTHIAZIDE\METOPROLOL:Anxiety:0.22), (HYDROC HLOROTHIAZIDE\METOPROLOL SUCCINATE:Anxiety:0.97), (HYDROCHLOROTHIAZIDE\PROPRANOLOL HYDROCHLORIDE:Anxiety:0.43), (HYDROCHLOROTHIAZIDE\RAMIPRIL:Anxiety:1.3), (HYDROCORTISONE HEMISUCCINATE:Anxiety:0.47), (HYDROCORTISONE\MICONAZOLE:Anxiety:0.57), (HYDROCORTISONE\NEOMYCIN SULFATE\POLYMYXIN B SULFATE:Anxiety:0.13), (HYDROXOCOBALAMIN ACETATE:Anxiety:0.27), (HYDROXOCOBALAMIN HYDROCHLORIDE:Anxiety:0.13), (INSULIN ASPART\INSULIN DEGLUDEC:Anxiety:0.26), (INSULIN PORK\INSULIN PURIFIED PORK:Anxiety:1.28), (INTERFERON ALFA-2A:Anxiety:2.71), (IODINE\POTASSIUM IODIDE:Anxiety:0.99), (IPRAGLIFLOZIN L-PROLINE:Anxiety:0), (ISAVUCONAZOLE:Anxiety:0.61), (ISAVUCONAZONIUM SULFATE:Anxiety:0.93), (ISOCARBOXAZID:Anxiety:0.31), (ISOFLAVONES SOY:Anxiety:0.43), (ISONIAZID\PYRAZINAMIDE\RIFAMPIN:Anxiety:1.85), (ISTRADEFY LLINE:Anxiety:0.63), (IXAZOMIB CITRATE:Anxiety:0.27), (JWH-018:Anxiety:0.1), (KERATIN:Anxiety:0.36), (LANOLIN\MINERAL OIL\PETROLATUM:Anxiety:0.46), (LARONIDASE:Anxiety:1.07), (LATANOPROST\TIMOL OL:Anxiety:1.15), (LETROZOLE\RIBOCICLIB:Anxiety:0.38), (LEVAMLODIPINE:Anxiety:0.47), (LEVOBUPIVACAINE:Anxiety:0.11), (LEVOBUPIVACAINE HYDROCHLORIDE:Anxiety:0.22), (LEVOMEFOLATE CALCIUM\METHYLCOBALAMIN\PYRIDOXAL 5-PHOSPHATE:Anxiety:0.32), (LEVOMEPROMAZINE HYDROCHLORIDE\LEVOMEPROMAZINE MALEATE:Anxiety:0.19), (LEVORPHANOL TARTRATE:Anxiety:0.44), (LEVOSIMENDAN:Anxiety:0.44), (LEVOTHYROXINE SODIUM\POTASSIUM IODIDE:Anxiety:1.32), (LINOLEIC ACID:Anxiety:0.02), (LIPASE:Anxiety:0.21), (LUTETIUM LU-177:Anxiety:1.61), (LYCOPENE:Anxiety:0.55), (LYNESTRENOL:Anxiety:0.26), (LYSERGIDE:Anxiety:0.24), (LYSINE HYDROCHLORIDE:Anxiety:0.37), (MAGNESIUM ASPARTATE HYDROCHLORIDE:Anxiety:1.74), (MAGNESIUM ASPARTATE\POTASSIUM ASPARTATE:Anxiety:1.29), (MAGNESIUM GLUCEPTATE:Anxiety:0.48), (MALIC ACID:Anxiety:0.04), (MANGANESE\MANGANESE CHLORIDE:Anxiety:0.75), (MANIDIPINE HYDROCHLORIDE:Anxiety:0.39), (MEFLOQUINE HYDROCHLORIDE:Anxiety:0.55), (METFORMIN HYDROCHLORIDE\SAXAGLIPTIN:Anxiety:1.42), (METHAZOLAMIDE:Anxiety:0.18), (ME THENAMINE MANDELATE:Anxiety:1.91), (METHENOLONE ENANTHATE:Anxiety:0.97), (METHYLENEDIOXYMETHAMPHETAMINE:Anxiety:0.42), ( METHYLERGONOVINE MALEATE:Anxiety:0.27), (METHYLPREDNISOLONE OR METHYLPREDNISOLONE ACETATE:Anxiety:0.75), (METYRAPONE:Anxiety:0.57), (METYROSINE:Anxiety:0.73), (MIF AMURTIDE:Anxiety:0.28), (MIGLUSTAT:Anxiety:2.02), (MINERALS\PROBIOTICS NOS\VITAMINS:Anxiety:0.13), (MIPOMERSEN SODIUM:Anxiety:0.52), (MITOTANE:Anxiety:0.14), (MIZORIBINE:Anxiety:0.1), (NADH:Anxiety: 0.63), (NIACINAMIDE\PYRIDOXINE\RIBOFLAVIN\THIAMINE:Anxiety:0.52), (NIFUR TOINOL:Anxiety:0.26), (NILUTAMIDE:Anxiety:0.71), (NUSINERSEN:Anxiety:1.15), (OBETI CHOLIC ACID:Anxiety:0.39), (OMEGA-6 FATTY ACIDS:Anxiety:1), (OPICAPONE:Anxiety:0.57), (OSTREA EDULIS SHELL:Anxiety:0.02), (OXETORONE:Anxiety:0.97), (PAMIDRONIC ACID:Anxiety:0.15), (PANCURONIUM:Anxiety:0.07), (PAPAYA:Anxiety:1.14), (PASCOPYR UM SMITHII POLLEN:Anxiety:0.17), (PASSIFLORA INCARNATA FLOWER:Anxiety:0.53), (PEGINTERFERON ALFA-2A OR PEGINTERFERON ALFA-2B OR PEGINTERFERON BETA-1A OR PEGINTERFERON LAMBDA-1A:Anxiety:0.02), (PEMOLINE:Anxiety:0.01), (PENICILLIN G BENZATHINE:Anxiety:0.32), (PENTAERYTHRITOL TETRANITRATE:Anxiety:0.88), (PENTOBARBITAL:Anxiety:0.47), (PERAZINE:Anxiety:0.25), (PEROSPIRONE:Anxiety:1.19), (PHENDIMETRAZINE:Anxiety:0.41), (PHENYLPROPANO LAMINE:Anxiety:0.14), (PIMAVANSERIN:Anxiety:0.3), (PIPERACILLIN SODIUM:Anxiety:0.05), (PIPOTIAZINE:Anxiety:0.37), (PIPOTIAZINE PALMITATE:Anxiety:0.43), (PIRARUBICIN:Anxiety:0.33), (PLANTAGO OVATA SEED COAT:Anxiety:0.13), (POLIDOCANOL:Anxiety:0.3), (PORFIMER SODIUM:Anxiety:0.64), (POTASSIUM BITARTRATE:Anxiety:0.31), (POTASSIUM BROMIDE:Anxiety:0.38), (POTASSIUM CHLORIDE\SODIUM CHLORIDE:Anxiety:0.45), (POTASSIUM IODIDE:Anxiety:1.41), (PRAMOXINE HYDROCHLORIDE:Anxiety:1.29), (PRANLUKAST:Anxiety:0.87), (PREDNISOLONE ACETATE\SULFACETAMIDE SODIUM:Anxiety:0.69), (PREDNISOLONE SODIUM METAZOATE:Anxiety:0.83), (PROMESTRIENE:Anxiety:0.06), (PROPANTHELINE:Anxiety: 0.41), (PROPARACAINE:Anxiety:0.13), (PROPIVERINE HYDROCHLORIDE:Anxiety:0.46), (PROPOXYPHENE NAPSYLATE:Anxiety:0.22), (PROTHIPENDYL HYDROCHLORIDE:Anxiety:0.11), (PSORALEN:Anxiety:0.49), (PYRIDOXAL PHOSPHATE:Anxiety:0.03), (QUINIDINE GLUCONATE:Anxiety:0.86), (RADIUM:Anxiety:0.36), (RASPBERRY KETONE:Anxiety:0.06), (RED CLOVER:Anxiety:1.07), (RHUBARB:Anxiety:0.36), (RILMAZAFONE:Anxiety:0.22), (RILON ACEPT:Anxiety:0.12), (RIMANTADINE HYDROCHLORIDE:Anxiety:0.18), (ROSIGLITAZONE:Anxiety:0.02), (SAFINAMIDE:Anxiety: 0.73), (SARPOGRELATE:Anxiety:1.21), (SEA WATER:Anxiety:0.24), (SEBELIPASE ALFA:Anxiety:0.35), (SECOBARBITAL SODIUM:Anxiety:0.02), (SENNOSIDE B:Anxiety:0.01), (SEROTONIN:Anxiety:0.28), (SERRAPEPTASE:Anxiety:0.58), (SHARK CARTILAGE\SHARK, UNSPECIFIED:Anxiety:0.34), (SHARK LIVER OIL:Anxiety:0.07), (SIBUTRAMINE:Anxiety:0.24), (SILICON DIOXIDE:Anxiety:1.75), (SILVER:Anxiety:0.16), (SODIUM CARBONATE:Anxiety:0.26), (SODIUM CITRATE:Anxiety:1.73), (SODIUM PHOSPHATE, MONOBASIC, ANHYDROUS:Anxiety:0.91), (SOLIFENACIN\TAMSULOSIN:Anxiety:0.28), (SOMATOSTA TIN:Anxiety:0.36), (SOMATREM:Anxiety:0.65), (SOY ISOFLAVONES:Anxiety:0.08), (STREPTOMYCIN\STREPTOMYCIN SULFATE:Anxiety:0.48), (STRONTIUM:Anxiety:0.34), (SULBUTIAMINE:Anxiety:0.78), (SUL FAMETHAZINE:Anxiety:0.2), (SULFANILAMIDE:Anxiety:0.02), (SULTHIAME:Anxiety:0.12), (SUPLATAST TOSILATE:Anxiety:0.37), (TABALUMAB:Anxiety:0.26), (TAZOBACTAM SODIUM:Anxiety:0.01), (TELITHROMYCIN:Anxiety:0.11), (TENOXICAM:Anxiety:0.85), (TE TRACAINE HYDROCHLORIDE:Anxiety:0.31), (THIAMYLAL SODIUM:Anxiety:0.48), (THIETHYLPERAZINE MALATE:Anxiety:0.25), (THIOPENTAL:Anxiety:1.32), (THIOPENTAL SODIUM:Anxiety:0.18), (THYMOL:Anxiety:0.02), (TIAGABINE:Anxiety:0.03), (TIAPROFENI C ACID:Anxiety:0.32), (TILIDINE:Anxiety:1.73), (TIOPRONIN:Anxiety:0.74), (TOLBUTAMIDE: Anxiety:0.05), (TOLMETIN:Anxiety:0.64), (TOLMETIN SODIUM:Anxiety:0.46), (TRIENTINE:Anxiety:0.18), (TRIENTINE HYDROCHLORIDE:Anxiety:0.38), (TRIMIPRAMINE:Anxiety:0.46), (TROGLITAZONE:Anxiety: 0.23), (UNDECYLENIC ACID:Anxiety:0.28), (URACIL:Anxiety:0.21), (VALERIAN EXTRACT:Anxiety:0.04), (VALRUBICIN:Anxiety:0.76), (VINBLASTINE SULFATE:Anxiety:0.24), (VINORELBINE\VINORELBINE TARTRATE:Anxiety:0.39), (VISCUM ALBUM FRUITING TOP:Anxiety:0.12), (VONOPRAZAN FUMURATE:Anxiety:0.25), (VORAPAXAR SULFATE:Anxiety:0.44), (XYLITOL:Anxiety:0.24), (YTTRIUM Y-90:Anxiety:0), (ZINC CITRATE:Anxiety:0.26), (ZOFENOPRIL:Anxiety:0.15), (ZUCLOPENTHIXOL DECANOATE:Anxiety:1.53), (ZUCLOPENTHIXOL HYDROCHLORIDE:Anxiety:0.58), (.ALPHA.1-PROTEINASE INHIBITOR HUMAN:Anxiety disorder:0.11), (ACARBOSE:Anxiety disorder:0.29), (ACETAMINOPHEN\OXYCODONE\OXYCODONE HYDROCHLORIDE:Anxiety disorder:0.31), (ACETAZOLAMIDE:Anxiety disorder:0.43), (ACTIVATED CHARCOAL:Anxiety disorder:0.12), (ALENDRONIC ACID:Anxiety disorder:0.37), (ALUMINUM HYDROXIDE\DIMETHICONE\MAGNESIUM HYDROXIDE:Anxiety disorder:0.07), (AMIODARONE:Anxiety disorder:1.61), (BENAZEPRIL HYDROCHLORIDE:Anxiety disorder:1.08), (BENAZEPRIL\BENAZEPRIL HYDROCHLORIDE:Anxiety disorder:0.03), (BENDAMUSTINE HYDROCHLORIDE:Anxiety disorder:0.2), (BETAMETHASONE DIPROPIONATE:Anxiety disorder:0.4), (BISACODYL OR DOCUSATE SODIUM:Anxiety disorder:0.7), (BRINZOLAMIDE:Anxiety disorder:0.24), (BUDESONIDE\FORMOTEROL FUMARATE:Anxiety disorder:0.24), (CALCIUM ACETATE:Anxiety disorder:0.44), (CALCIUM CITRATE:Anxiety disorder:0.54), (CANAGLIFLOZIN\METFORMIN HYDROCHLORIDE:Anxiety disorder:0.29), (CANDESARTAN CILEXETIL\HYDROCHLOROTHIAZIDE:Anxiety disorder:1.51), (CAPTOPRIL:Anxiety disorder:0.31), (CARVEDILOL PHOSPHATE:Anxiety disorder:0.64), (CHONDROITIN SULFATE (BOVINE)\GLUCOSAMINE HYDROCHLORIDE:Anxiety disorder:0.03), (CICLESONIDE:Anxiety disorder:0.11), (CILASTATIN SODIUM\IMIPENEM:Anxiety disorder:0.53), (CLOTRIMAZOLE:Anxiety disorder:0.82), (DACLIZUMAB:Anxiety disorder:0.69), (DALTEPARIN:Anxiety disorder:0.08), (DASATINIB:Anxiety disorder:1.74), (DEFLAZACORT:Anxiety disorder:0.11), (DIGITOXIN:Anxiety disorder:0.08), (DIPYRIDAMOLE:Anxiety disorder:0.43), (DOCUSATE:Anxiety disorder:0.68), (DOCUSATE CALCIUM:Anxiety disorder:0.51), (DUTASTERIDE\TAMSULOSIN HYDROCHLORIDE:Anxiety disorder:0.26), (EDOXABAN TOSYLATE:Anxiety disorder:1.01), (EFAVIRENZ\EMTRICITABINE\TENOFOVIR DISOPROXIL FUMARATE:Anxiety disorder:0.02), (EMPAGLIFLOZIN\LINAGLIPTIN:Anxiety disorder:0.17), (EPLERENONE:Anxiety disorder:0.53), (ESTROGENS, CONJUGATED\MEDROXYPROGESTERONE ACETATE:Anxiety disorder:0.7), (ETHINYL ESTRADIOL\NORGESTIMATE:Anxiety disorder:0.18), (FELODIPINE:Anxiety disorder:0.05), (FEXOFENADINE\PSEUDOEPHEDRINE:Anxiety disorder:0.08), (FISH OIL\OMEGA-3 FATTY ACIDS:Anxiety disorder:0.11), (FORMOTEROL FUMARATE:Anxiety disorder:0.33), (GUANFACINE:Anxiety disorder:0.89), (HYDROXYUREA:Anxiety disorder:0.29), (HYOSCYAMINE:Anxiety disorder:0.1), (IMIPRAMINE HYDROCHLORIDE:Anxiety disorder:0.86), (INDAPAMIDE\PERINDOPRIL:Anxiety disorder:0.87), (INTERFERON:Anxiety disorder:0.35), (INTERFERON ALFA-2A:Anxiety disorder:0.13), (IPRATROPIUM:Anxiety disorder:1.06), (ISOSORBIDE:Anxiety disorder:0.76), (IXAZOMIB:Anxiety disorder:0.22), (LABETALOL HYDROCHLORIDE:Anxiety disorder:0.11), (LABETALOL\LABETALOL HYDROCHLORIDE:Anxiety disorder:0.7), (LIDOCAINE\PRILOCAINE:Anxiety disorder:0.31), (LINACLOTIDE:Anxiety disorder:0.1), (METAXALONE:Anxiety disorder:0.65), (METFORMIN HYDROCHLORIDE\VILDAGLIPTIN:Anxiety disorder:0.15), (METHENAMINE HIPPURATE:Anxiety disorder:0.33), (METHYLPREDNISOLONE ACETATE:Anxiety disorder:0.34), (MOXIFLOXACIN:Anxiety disorder:0.04), (MOXIFLOXACIN HYDROCHLORIDE:Anxiety disorder:1.04), (MOXONIDINE:Anxiety disorder:0.21), (NABUMETONE:Anxiety disorder:0.14), (NADROPARIN CALCIUM:Anxiety disorder:0.06), (NALOXONE HYDROCHLORIDE\OXYCODONE HYDROCHLORIDE:Anxiety disorder:0.85), (NICOTINE:Anxiety disorder:0.54), (NORETHINDRONE:Anxiety disorder:0.29), (OSELTAMIVIR PHOSPHATE:Anxiety disorder:0.09), (PANTOPRAZOLE MAGNESIUM:Anxiety disorder:1.05), (PEGINTERFERON BETA-1A:Anxiety disorder:0.82), (PENTOSAN POLYSULFATE SODIUM:Anxiety disorder:0.21), (PILSICAINIDE HYDROCHLORIDE:Anxiety disorder:0.42), (PIROXICAM:Anxiety disorder:0.37), (PONATINIB:Anxiety disorder:0.19), (PRIMIDONE:Anxiety disorder:1.16), (RALTEGRAVIR POTASSIUM:Anxiety disorder:0.29), (RANOLAZINE:Anxiety disorder:1.28), (REGORAFENIB:Anxiety disorder:0.14), (RIVASTIGMINE TARTRATE:Anxiety disorder:0.32), (ROFLUMILAST:Anxiety disorder:0.62), (ROSUVASTATIN:Anxiety disorder:0.08), (SACUBITRIL\VALSARTAN:Anxiety disorder:1.49), (SECUKINUMAB:Anxiety disorder:1.11), (SITAGLIPTIN:Anxiety disorder:0.64), (SOLIFENACIN SUCCINATE:Anxiety disorder:0.58), (SOTALOL:Anxiety disorder:1.18), (TESTOSTERONE UNDECANOATE:Anxiety disorder:0.07), (TETRACYCLINE:Anxiety disorder:0.11), (TOBRAMYCIN:Anxiety disorder:0.07), (TOCILIZUMAB:Anxiety disorder:0.37), (TOLTERODINE TARTRATE:Anxiety disorder:0.7), (TRIHEXYPHENIDYL:Anxiety disorder:0.37), (UMECLIDINIUM BROMIDE\VILANTEROL TRIFENATATE:Anxiety disorder:0.64), (VALGANCICLOVIR HYDROCHLORIDE:Anxiety disorder:0.17), (VITAMIN A:Anxiety disorder:0.18), (WARFARIN POTASSIUM:Anxiety disorder:0.06), (ZINC:Anxiety disorder:0.21), (ACETAMINOPHEN\BUTALBITAL\CAFFEINE:Anxiolytic therapy:0.1), (ALBUTEROL SULFATE\IPRATROPIUM BROMIDE:Anxiolytic therapy:0.11), (BENAZEPRIL HYDROCHLORIDE:Anxiolytic therapy:0.05), (BUMETANIDE:Anxiolytic therapy:0.77), (BUPRENORPHINE HYDROCHLORIDE\NALOXONE HYDROCHLORIDE:Anxiolytic therapy:0.28), (CANDESARTAN:Anxiolytic therapy:0.8), (CEPHALEXIN:Anxiolytic therapy:0.43), (CIPROFLOXACIN HYDROCHLORIDE:Anxiolytic therapy:0.24), (COLCHICINE:Anxiolytic therapy:0.31), (CYCLOBENZAPRINE HYDROCHLORIDE:Anxiolytic therapy:0.25), (DALFAMPRIDINE:Anxiolytic therapy:1), (DASATINIB:Anxiolytic therapy:0.47), (DESLORATADINE:Anxiolytic therapy:0.35), (DEXLANSOPRAZOLE:Anxiolytic therapy:0.73), (DICLOFENAC SODIUM:Anxiolytic therapy:0.4), (DILTIAZEM:Anxiolytic therapy:0.48), (DONEPEZIL:Anxiolytic therapy:0.12), (DOXAZOSIN\DOXAZOSIN MESYLATE:Anxiolytic therapy:0.15), (ELETRIPTAN HYDROBROMIDE:Anxiolytic therapy:0.07), (ESZOPICLONE:Anxiolytic therapy:0.38), (ETORICOXIB:Anxiolytic therapy:0.27), (EVOLOCUMAB:Anxiolytic therapy:0.66), (FERROUS SULFATE:Anxiolytic therapy:0.85), (FLUTICASONE PROPIONATE:Anxiolytic therapy:0.84), (GLIPIZIDE:Anxiolytic therapy:0.07), (GLYCOPYRRONIUM\INDACATEROL:Anxiolytic therapy:0.06), (HYDROCHLOROTHIAZIDE\LISINOPRIL:Anxiolytic therapy:0.48), (INDAPAMIDE\PERINDOPRIL:Anxiolytic therapy:0.41), (INDOMETHACIN:Anxiolytic therapy:0.23), (IPRATROPIUM:Anxiolytic therapy:0.13), (LACOSAMIDE:Anxiolytic therapy:0.17), (LISDEXAMFETAMINE DIMESYLATE:Anxiolytic therapy:0.13), (METHYLPREDNISOLONE ACETATE:Anxiolytic therapy:0.49), (MODAFINIL:Anxiolytic therapy:0.06), (NAPROXEN SODIUM:Anxiolytic therapy:0.01), (PANTOPRAZOLE MAGNESIUM:Anxiolytic therapy:0.11), (PENICILLIN:Anxiolytic therapy:0.12), (PRIMIDONE:Anxiolytic therapy:0.43), (RANITIDINE HYDROCHLORIDE:Anxiolytic therapy:0.57), (RANOLAZINE:Anxiolytic therapy:0.49), (ROPINIROLE HYDROCHLORIDE:Anxiolytic therapy:0.31), (SCOPOLAMINE:Anxiolytic therapy:0.01), (SOTALOL:Anxiolytic therapy:0.4), (TERIFLUNOMIDE:Anxiolytic therapy:0.25), (THALIDOMIDE:Anxiolytic therapy:0.26), (TOFACITINIB CITRATE:Anxiolytic therapy:0.07), (TORSEMIDE:Anxiolytic therapy:0.45), (TRIAMCINOLONE ACETONIDE:Anxiolytic therapy:0.24), (RAMIPRIL:Aortic aneurysm:0.31), (VERAPAMIL HYDROCHLORIDE:Aortic aneurysm:0.01), (CERTOLIZUMAB PEGOL:Aortic valve replacement:0.34), (DABIGATRAN:Aortic valve replacement:0.21), (DASATINIB:Aortic valve replacement:0.03), (DEVICE:Aortic valve replacement:0.06), (ERYTHROPOIETIN:Aortic valve replacement:0.07), (EVEROLIMUS:Aortic valve replacement:0.62), (HYDROCHLOROTHIAZIDE\LISINOPRIL:Aortic valve replacement:0.03), (ILOPROST:Aortic valve replacement:0.36), (INDAPAMIDE:Aortic valve replacement:0.1), (INDAPAMIDE\PERINDOPRIL:Aortic valve replacement:0.34), (INSULIN HUMAN:Aortic valve replacement:0.14), (LORATADINE:Aortic valve replacement:0.22), (METAMIZOLE:Aortic valve replacement:0.05), (METHYLPREDNISOLONE SODIUM SUCCINATE:Aortic valve replacement:0.41), (METRONIDAZOLE:Aortic valve replacement:0.61), (PAROXETINE\PAROXETINE HYDROCHLORIDE:Aortic valve replacement:0.02), (PRAVASTATIN:Aortic valve replacement:0.47), (TOCILIZUMAB:Aortic valve replacement:0.14), (TOFACITINIB CITRATE:Aortic valve replacement:0.16), (ZONISAMIDE:Aortic valve replacement:0.06), (BUMETANIDE:Aplastic anaemia:0.18), (CALCITRIOL:Aplastic anaemia:0.05), (CANAKINUMAB:Aplastic anaemia:0.03), (DIVALPROEX SODIUM:Aplastic anaemia:0.09), (DOCUSATE:Aplastic anaemia:0.05), (ENALAPRIL:Aplastic anaemia:0.76), (ENOXAPARIN:Aplastic anaemia:0.7), (ESZOPICLONE:Aplastic anaemia:0.03), (FERROUS SULFATE\FERROUS SULFATE, DRIED:Aplastic anaemia:0.54), (IMATINIB MESYLATE:Aplastic anaemia:0.25), (IRON SUCROSE:Aplastic anaemia:0), (MOXIFLOXACIN:Aplastic anaemia:0.17), (NAPROXEN:Aplastic anaemia:0.05), (NEBIVOLOL:Aplastic anaemia:0.19), (OSELTAMIVIR:Aplastic anaemia:0.22), (OXCARBAZEPINE:Aplastic anaemia:0.37), (PONATINIB:Aplastic anaemia:0.09), (VERAPAMIL HYDROCHLORIDE:Aplastic anaemia:0.14), (WARFARIN SODIUM:Aplastic anaemia:0.25), (DICLOFENAC:Apnoea:0.03), (DIGOXIN:Apnoea:0.04), (DIVALPROEX SODIUM:Apnoea:0.63), (ENALAPRIL:Apnoea:0.04), (EVOLOCUMAB:Apnoea:0.06), (LOVAS TATIN:Apnoea:0.38), (PANTOPRAZOLE:Apnoea:0.05), (PHENOBARBITAL:Apnoea:0.06), (R AMIPRIL:Apnoea:0.07), (ROSUVASTATIN CALCIUM:Apnoea:0.14), (TERIFLUNOMIDE:Apnoea:0.15), (TIOTROPIUM BROMIDE MONOHYDRATE:Apnoea:0.18), (VARENICLINE TARTRATE:Apnoea:0.33), (VERAPAMIL HYDROCHLORIDE:Apnoea:0.02), (WARFARIN:Apnoea:0.3), (ZONISAMIDE:Apnoea:0.04), (ADO-TRASTUZUMAB EMTANSINE:Appetite disorder:0.12), (AMOXICILLIN:Appetite disorder:0.4), (BICALUTAMIDE:Appetite disorder:0.47), (BISOPROLOL:Appetite disorder:0.06), (BUPRENORPHINE HYDROCHLORIDE\NALOXONE HYDROCHLORIDE:Appetite disorder:0.3), (CALCIUM ACETATE:Appetite disorder:0.1), (CANDESARTAN CILEXETIL:Appetite disorder:0.23), (CEPHALEXIN:Appetite disorder:0.67), (CORTISONE\HYDROCORTISONE:Appetite disorder:0.17), (DACLIZUMAB:Appetite disorder:0.03), (DASATINIB:Appetite disorder:0.74), (DEVICE:Appetite disorder:0.21), (DOXAZOSIN MESYLATE:Appetite disorder:0.05), (DOXYCYCLINE:Appetite disorder:0.05), (ELETRIPTAN HYDROBROMIDE:Appetite disorder:0.4), (ESOMEPRAZOLE:Appetite disorder:0.2), (ESZOPICLONE:Appetite disorder:0.68), (EVOLOCUMAB:Appetite disorder:0.51), (FERROUS SULFATE\FERROUS SULFATE, DRIED:Appetite disorder:0.91), (FLUOXETINE\FLUOXETINE HYDROCHLORIDE:Appetite disorder:0.1), (FLUTICASONE:Appetite disorder:0.1), (FLUTICASONE FUROATE\VILANTEROL TRIFENATATE:Appetite disorder:0.35), (HYDROCHLOROTHIAZIDE\LISINOPRIL:Appetite disorder:0.56), (HYDROXYUREA:Appetite disorder:0.33), (HYDROXYZINE PAMOATE:Appetite disorder:0.27), (HYOSCYAMINE:Appetite disorder:0.03), (ILOPROST:Appetite disorder:0.09), (LACOSAMIDE:Appetite disorder:0.2), (LINAGLIPTIN:Appetite disorder:0.04), (LITHIUM CARBONATE:Appetite disorder:0.1), (METAXALONE:Appetite disorder:0.42), (METHENAMINE HIPPURATE:Appetite disorder:0.02), (METRONIDAZOLE:Appetite disorder:1.04), (MOXIFLOXACIN HYDROCHLORIDE:Appetite disorder:0.1), (NEBIVOLOL HYDROCHLORIDE:Appetite disorder:0.02), (NIFEDIPINE:Appetite disorder:0.12), (NILOTINIB:Appetite disorder:0.51), (PANTOPRAZOLE MAGNESIUM:Appetite disorder:0.32), (PAROXETINE:Appetite disorder:0.1), (PENICILLIN:Appetite disorder:0.01), (PHENYTOIN:Appetite disorder:0.4), (PONATINIB:Appetite disorder:0.27), (PRAMIPEXOLE DIHYDROCHLORIDE:Appetite disorder:0.47), (PRIMIDONE:Appetite disorder:0.5), (QUETIAPINE:Appetite disorder:0.33), (RALOXIFENE HYDROCHLORIDE:Appetite disorder:0.17), (RANOLAZINE:Appetite disorder:0.61), (REGORAFENIB:Appetite disorder:0.07), (ROPINIROLE HYDROCHLORIDE:Appetite disorder:0.03), (SACUBITRIL\VALSARTAN:Appetite disorder:0.45), (SUMATRIPTAN\SUMATRIPTAN SUCCINATE:Appetite disorder:0.11), (TELMISARTAN:Appetite disorder:0.19), (TRIAMCINOLONE ACETONIDE:Appetite disorder:0.29), (.ALPHA.1-PROTEINASE INHIBITOR HUMAN:Arrhythmia:0.78), (ABOBOTULINUMTOXINA:Arrhythmia:0.02), (ACETAMINOPH EN\OXYCODONE\OXYCODONE HYDROCHLORIDE:Arrhythmia:1.6), (ACITRETIN:Arrhythmia:0.52), (ADO-TRASTUZUMAB EMTANSINE:Arrhythmia:1.54), (ALBIGLUTIDE:Arrhythmia:0.21), (ALCOHOL:Arrhythmia:0.81), (ALEMTUZUMAB:Arrhythmia:0.52), (ALISKIREN HEMIFUMARATE:Arrhythmia:1.06), (ALOGLIPTIN:Arrhythmia:0.04), (ALOGLIPTIN BENZOATE\PIOGLITAZONE HYDROCHLORIDE:Arrhythmia:0), (ALUMINUM HYDROXIDE\MAGNESIUM OXIDE\OXETHAZAINE:Arrhythmia:0.02), (AMBROXOL:Arrhythmia:0.67), (AMBROXOL\C LENBUTEROL:Arrhythmia:0.17), (AMINO ACIDS\DEXTROSE\ELECTROLYTES NOS:Arrhythmia:0.44), (AMISULPRIDE:Arrhythmia:1.56), (AMOXICILLIN\CLARITHROMY CIN\ESOMEPRAZOLE:Arrhythmia:0.17), (AMPICILLIN:Arrhythmia:0.95), (AMPICILLIN SODIUM:Arrhythmia:0.04), (ANTIHISTAMINES NOS:Arrhythmia:0.35), (ASUNAPREVIR:Arrhythmia:1.06), (ATROPINE\DIPHENOXYLATE: Arrhythmia:0.31), (AZELNIDIPINE:Arrhythmia:1.16), (AZITHROMYCIN OR METHOTREXATE SODIUM:Arrhythmia:0.42), (BACITRACIN:Arrhythmia:0.4), (BAMIFYLLINE HYDROCHLORIDE:Arrhythmia:0.06), (BECLOMETHASONE:Arrhythmia:0.51), (BECLOME THASONE\FORMOTEROL:Arrhythmia:0.4), (BELATACEPT:Arrhythmia:0.74), (BENIDIPIN E:Arrhythmia:0.73), (BENZTROPINE MESYLATE:Arrhythmia:0.32), (BERAPROST:Arrhythmia:0.31), (BETAMETHASONE DIPROPIONATE:Arrhythmia:1.12), (BETAMETHASONE DIPROPIONATE\CALCIPOTRIENE:Arrhythmia:0.17), (BETAMETHASONE DIPROPIONATE\CALCIPOTRIENE\CALCIPOTRIENE HYDRATE:Arrhythmia:0.62), (BETAMETHASONE DIPROPIONATE\CLOTRIMAZOLE:Arrhythmia:0.76), (BETAMETHASONE\CALCIPOTRIE NE:Arrhythmia:0.48), (BLINATUMOMAB:Arrhythmia:0.62), (BOSUTINIB:Arrhythmia:0.06), (BOTULINUM TOXIN TYPE A:Arrhythmia:0.19), (BUPRENORPHINE HYDROCHLORIDE\NALOXONE HYDROCHLORIDE:Arrhythmia:0.66), (CALCITONIN:Arrhythmia:0.87), (CALCIUM CARBONATE\ERGOCALCIFEROL\RETINOL:Arrhythmia:0.43), (CALCIUM CARBONATE\VITAMIN D:Arrhythmia:1.35), (CALCIUM LACTATE:Arrhythmia:0.25), (CALCIUM\MAGNESIUM\ZINC:Arrhythmia:0.4), (CANAGLIF LOZIN\METFORMIN HYDROCHLORIDE:Arrhythmia:1.66), (CANNABIS SATIVA SUBSP. \INDICA TOP:Arrhythmia:0.12), (CAPSAICIN:Arrhythmia:0.02), (CARBASALATE:Arrhythmia:0.13), (C ARBIDOPA:Arrhythmia:0.67), (CARBOHYDRATES\FATTY ACIDS\MINERALS\PROTEIN\VITAMINS:Arrhythmia:0.28), (CARTEOLOL:Arrhythmia:0.31), (CASANTHRANOL\DOCUSATE SODIUM:Arrhythmia:0.63), (CEFAZOLIN SODIUM:Arrhythmia:1.26), (CEFEPIME\CEFEPIME HYDROCHLORIDE:Arrhythmia:0.53), (CERITINIB:Arrhythmia:0.96), (CHLORHEXIDINE GLUCONATE:Arrhythmia:0.5), (CHLOROQUINE PHOSPHATE:Arrhythmia:0.26), (CLENBUTEROL HYDROCHLORIDE:Arrhythmia:0.03), (CLOTRIMAZOLE:Arrhythmia:2.08), (COLISTIMETH ATE SODIUM:Arrhythmia:0.13), (CYPROHEPTADINE:Arrhythmia:0.77), (DABIGATRAN ETEXILATE:Arrhythmia:0.5), (DACLIZUMAB:Arrhythmia:10.65), (DANAZOL:Arrhythmia:1.46), (DANTROLENE SODIUM:Arrhythmia:0.18), (DAPAGLIFLOZIN PROPANEDIOL\METFORMIN HYDROCHLORIDE:Arrhythmia:0.25), (DECITABINE:Arrhythmia:0.12), (DEGARELIX:Arrhythmia: 0.66), (DESONIDE:Arrhythmia:0.75), (DEXTROAMPHETAMINE SULFATE:Arrhythmia:0.61), (DIFLUPREDNATE:Arrhythmia:0.04), (DIPHENIDOL HYDROCHLORIDE:Arrhythmia:0.03), (DIPYRONE\PROMETHAZINE HYDROCHLORIDE:Arrhythmia:0.19), (DOLASETRON MESYLATE:Arrhythmia:0.31), (ELTROMBOPAG OLAMINE:Arrhythmia:0.63), (EMPAGLIFLOZIN\LINAGLIPTIN:Arrhythmia:1.05), (EPIRUBI CIN:Arrhythmia:1.35), (ERIBULIN:Arrhythmia:0.59), (ETHACRYNIC ACID:Arrhythmia:0.36), (ETRETINATE:Arrhythmia:0.31), (FERRIC HYDROXIDE:Arrhythmia:1.12), (FERROUS GLYCINE SULFATE:Arrhythmia:0), (FERROUS SULFATE\GLYCINE:Arrhythmia:0.78), (FEXOFENADINE\PSEUDOEPHEDRINE:Arrhythmia: 0.58), (FLUDROCORTISONE ACETATE:Arrhythmia:1.54), (FLURAZEPAM\FLURAZEPAM HYDROCHLORIDE:Arrhythmia:0.3), (FLUVOXAMINE:Arrhythmia:0.04), (FLUVOXAMINE MALEATE:Arrhythmia:0.93), (FORMOTEROL FUMARATE\MOMETASONE FUROATE:Arrhythmia:0.81), (FOSPHENYTOIN\FOSPHENYTOIN SODIUM:Arrhythmia:0.02), (FULVESTRANT:Arrhythmia:0.77), (GENTAMICIN SULFATE:Arrhythmia:0.22), (GLYCERIN:Arrhythmia:0.57), (GLYCOPYRROLATE\INDACA TEROL MALEATE:Arrhythmia:0.76), (HERBALS\TURMERIC:Arrhythmia:0.96), (HYDROCHLOROT HIAZIDE\SPIRONOLACTONE:Arrhythmia:0.24), (HYDROCODONE BITARTRATE\IBUPROFEN:Arrhythmia:0.33), (HYDROXYCHLOROQUINE DIPHOSPHATE:Arrhythmia:0.88), (HYOSCYAMINE SULFATE:Arrhythmia:0.19), (IBRITUMOMAB TIUXETAN:Arrhythmia:0.45), (IMIGLUCERASE:Arrhythmia:0.69), (INFLIXIMAB-DYYB:Arrhythmia:0.56), (INSULIN PORK\INSULIN PURIFIED PORK:Arrhythmia:0.32), (INTERFERON:Arrhythmia:0.96), (INTERFERON ALFA-2A:Arrhythmia:1.43), (INTERFERON ALFA-2B:Arrhythmia:0.77), (IRINOTECAN HYDROCHLORIDE:Arrhythmia:1.25), (KETOROLAC\KETOROLAC TROMETHAMINE:Arrhythmia:0.65), (LABETALOL HYDROCHLORIDE:Arrhythmia:1.84), (LAPATINIB DITOSYLATE:Arrhythmia:0.58), (LEVOCETIRIZINE:Arrhythmia:0.27), (LEVOLEUCOVORI N CALCIUM:Arrhythmia:0.7), (LEVOSIMENDAN:Arrhythmia:0.5), (LIDOCAINE\PRILOCAIN E:Arrhythmia:0.9), (LINSIDOMINE:Arrhythmia:0.75), (LIOTHYRONINE:Arrhythmia:0.43), (LI SDEXAMFETAMINE DIMESYLATE:Arrhythmia:0.83), (LOMUSTINE:Arrhythmia:0.15), (LOXOPROFEN:Arrhythmia: 0.36), (LURASIDONE HYDROCHLORIDE:Arrhythmia:0.29), (LUTETIUM LU-177:Arrhythmia:0.15), (MAGNESIUM ASPARTATE HYDROCHLORIDE:Arrhythmia:0.87), (MEDROXYPROGESTERONE:Arrhythmia:0.03), (ME FENAMIC ACID:Arrhythmia:0.46), (MEGESTROL:Arrhythmia:0.4), (MELPERONE:Arrhythmia:0.88), (M EPERIDINE:Arrhythmia:1.63), (MEPOLIZUMAB:Arrhythmia:0.61), (MESNA:Arrhythmia:0.97), (METFORMIN HYDROCHLORIDE\ROSIGLITAZONE MALEATE:Arrhythmia:1.1), (METHENAMINE HIPPURATE:Arrhythmia:1.11), (METHENOLONE ENANTHATE:Arrhythmia:0.45), (METHYLPREDNISOLONE ACETATE:Arrhythmia:1.72), (METRELEPTIN:Arrhythmia:0.34), (MIGLUSTAT:Arrhythmia:0.18), (MILK THISTLE:Arrhythmia:0.44), (MILRINONE LACTATE:Arrhythmia:0.07), (MIZORIBINE:Arrhythmia:0.49), (MOEXIPRIL HYDROCHLORIDE:Arrhythmia:0.6), (MORPHINE SULFATE\NALTREXONE HYDROCHLORIDE:Arrhythmia:0.42), (NALOXONE:Arrhythmia:0.18), (NARATRIPTAN HYDROCHLORIDE:Arrhythmia:0.03), (NORETHINDRONE:Arrhythmia:0.31), (OXYBUTYNI N\OXYBUTYNIN CHLORIDE:Arrhythmia:0.35), (PARICALCITOL:Arrhythmia:1.46), (PEGINTERFERON BETA-1A:Arrhythmia:1.22), (PENTOSTATIN:Arrhythmia:0.36), (PILOCARPINE:Arrhythmia:0.73), (P IRITRAMIDE:Arrhythmia:0.8), (POLYETHYLENE GLYCOL 3350\POTASSIUM CHLORIDE\SODIUM BICARBONATE\SODIUM CHLORIDE\SODIUM SULFATE ANHYDROUS:Arrhythmia:0.58), (POLYSTYRENE:Arrhythmia:0.34), (POVIDONE:Arrhythmia: 0.18), (PRANLUKAST:Arrhythmia:0.34), (PREDNISOLONE SODIUM PHOSPHATE:Arrhythmia:0.66), (PYRAZINAMIDE:Arrhythmia:0.78), (RADIATION THERAPY:Arrhythmia:1.56), (RADIUM RA-223 DICHLORIDE:Arrhythmia:0.29), (REGADENOSON:Arrhythmia:0.03), (RIZATRIPTAN:Arrhythmia: 0.12), (ROSIGLITAZONE MALEATE:Arrhythmia:1.23), (SCOPOLAMINE:Arrhythmia:1.68), (SCOPOLAMINE HYDROBROMIDE:Arrhythmia:0.18), (SELEGILINE:Arrhythmia:0.26), (SEVELAMER:Arrhythmia: 0.37), (SIPULEUCEL-T:Arrhythmia:0.34), (STARCH, WHEAT:Arrhythmia:0.01), (SUMATRIPTAN:Arrhythmia:1.01), (SUMATRIPTAN\SUMATRIP TAN SUCCINATE:Arrhythmia:1.59), (TELOTRISTAT ETHYL:Arrhythmia:0.27), (TERAZOSIN HYDROCHLORIDE:Arrhythmia:1.08), (TESTOSTERONE ENANTHATE:Arrhythmia:0.28), (TESTOSTERONE UNDECANOATE:Arrhythmia:1.55), (THIOCTIC ACID:Arrhythmia:0.98), (TIGECYCLINE:Arrhythmia:0.61), (TIMOLOL\TRAVOPROST:Arrhythmia: 0.5), (TRAMETINIB DIMETHYL SULFOXIDE:Arrhythmia:0.32), (TRIHEXYPHENIDYL:Arrhythmia:0.44), (TRIMIPRAMINE MALEATE:Arrhythmia:1.14), (TRIPTORELIN PAMOATE:Arrhythmia:0.68), (UROKINASE:Arrhythmia:0.33), (VALDECOXIB:Arrhythmia:0.13), (XIPAMIDE:Arrhythmia:0.62), (ZILEUTON:Arrhythmia:0.4), (ZINC:Arrhythmia:1.24), (ZIV AFLIBERCEPT:Arrhythmia:0.06), (ZOFENOPRIL:Arrhythmia:0.01), (AMITRIPTYLINE:Arrhy thmia prophylaxis:0.12), (ANASTROZOLE:Arrhythmia prophylaxis:0.23), (CALCIUM ACETATE:Arrhythmia prophylaxis:0.09), (CANDESARTAN CILEXETIL:Arrhythmia prophylaxis:0.69), (CERTOLIZUMAB PEGOL:Arrhythmia prophylaxis:0.07), (CHOLESTYRAMINE:Arrhythmia prophylaxis:0.14), (DASATINIB:Arrhythmia prophylaxis:0.36), (DEXLANSOPRAZOLE:Arrhythmia prophylaxis:0.31), (DICLOFENAC:Arrhythmia prophylaxis:0.06), (DICLOFENAC SODIUM:Arrhythmia prophylaxis:0.22), (DIPHENHYDRAMINE:Arrhythmia prophylaxis:0.56), (DIPHENHYDRAMINE HYDROCHLORIDE:Arrhythmia prophylaxis:0), (ESZOPICLONE:Arrhythmia prophylaxis:0.05), (EVOLOCUMAB:Arrhythmia prophylaxis:0.41), (FEBUXOSTAT:Arrhythmia prophylaxis:0.03), (HYDROCHLOROTHIAZIDE\LISINOPRIL:Arrhythmia prophylaxis:0.19), (ILOPROST:Arrhythmia prophylaxis:0.03), (IMATINIB MESYLATE:Arrhythmia prophylaxis:0.37), (INDAPAMIDE\PERINDOPRIL:Arrhythmia prophylaxis:0.07), (IRBESARTAN:Arrhythmia prophylaxis:0.34), (LEDIPASVIR\SOFOSBUVIR:Arrhythmia prophylaxis:0.24), (LERCANIDIPINE:Arrhythmia prophylaxis:0.58), (LERCANIDIPINE HYDROCHLORIDE:Arrhythmia prophylaxis:0.21), (LOVASTATIN:Arrhythmia prophylaxis:0.8), (METRONIDAZOLE:Arrhythmia prophylaxis:0.44), (NILOTINIB:Arrhythmia prophylaxis:0.7), (NYSTATIN:Arrhythmia prophylaxis:0.11), (OMEGA-3-ACID ETHYL ESTERS:Arrhythmia prophylaxis:0.1), (OXCARBAZEPINE:Arrhythmia prophylaxis:0.21), (OXYGEN:Arrhythmia prophylaxis:0.54), (PHENOBARBITAL:Arrhythmia prophylaxis:0.03), (PRAVASTATIN SODIUM:Arrhythmia prophylaxis:0.57), (TRIAMCINOLONE ACETONIDE:Arrhythmia prophylaxis:0.05), (CALCITRIOL:Arterial disorder:0.24), (CERTOLIZUMAB PEGOL:Arterial disorder:0.07), (DICLOFENAC:Arterial disorder:0.25), (DOCUSATE SODIUM:Arterial disorder:0.06), (EVEROLIMUS:Arterial disorder:0.06), (EVOLOCUMAB:Arterial disorder:0.88), (FLECAINIDE:Arterial disorder:0.22), (ILOPROST:Arterial disorder:0.68), (IRBESARTAN:Arterial disorder:0.11), (MELOXICAM:Arterial disorder:0.34), (NAPROXEN:Arterial disorder:0.84), (RALOXIFENE HYDROCHLORIDE:Arterial disorder:0.19), (SACUBITRIL\VALSARTAN:Arterial disorder:0.46), (TAMSULOSIN HYDROCHLORIDE:Arterial disorder:0.02), (ACETAMINOPHEN\CODEINE PHOSPHATE:Arterial occlusive disease:0.41), (BISACODYL OR DOCUSATE SODIUM:Arterial occlusive disease:0.12), (BUMETANIDE:Arterial occlusive disease:0.53), (CALCIUM ACETATE:Arterial occlusive disease:0.23), (CALCIUM\VITAMIN D:Arterial occlusive disease:0.04), (CANDESARTAN:Arterial occlusive disease:0.53), (CANDESARTAN CILEXETIL\HYDROCHLOROTHIAZIDE:Arterial occlusive disease:0.35), (CARVEDILOL PHOSPHATE:Arterial occlusive disease:0.22), (CERTOLIZUMAB PEGOL:Arterial occlusive disease:0.54), (COLESEVELAM HYDROCHLORIDE:Arterial occlusive disease:0), (CYCLOBENZAPRINE HYDROCHLORIDE:Arterial occlusive disease:0.15), (DABIGATRAN:Arterial occlusive disease:0.05), (DAPAGLIFLOZIN PROPANEDIOL:Arterial occlusive disease:0.18), (DASATINIB:Arterial occlusive disease:0.1), (DOXAZOSIN\DOXAZOSIN MESYLATE:Arterial occlusive disease:0.35), (DUTASTERIDE:Arterial occlusive disease:0.18), (ENALAPRIL:Arterial occlusive disease:1.73), (EPLERENONE:Arterial occlusive disease:0.01), (ERGOCALCIFEROL:Arterial occlusive disease:0.31), (ESZOPICLONE:Arterial occlusive disease:0.03), (FLECAINIDE:Arterial occlusive disease:0.07), (IBRUTINIB:Arterial occlusive disease:1.34), (INSULIN DETEMIR:Arterial occlusive disease:0.75), (INSULIN GLULISINE:Arterial occlusive disease:0.09), (LABETALOL\LABETALOL HYDROCHLORIDE:Arterial occlusive disease:0.1), (LERCANIDIPINE:Arterial occlusive disease:0.46), (MELATONIN:Arterial occlusive disease:0.13), (METHYLPREDNISOLONE SODIUM SUCCINATE:Arterial occlusive disease:0.13), (METRONIDAZOLE:Arterial occlusive disease:0.25), (NILOTINIB:Arterial occlusive disease:0.32), (OMEGA-3-ACID ETHYL ESTERS:Arterial occlusive disease:0.38), (OXYGEN:Arterial occlusive disease:0.68), (PANTOPRAZOLE MAGNESIUM:Arterial occlusive disease:0.1), (PRAMIPEXOLE DIHYDROCHLORIDE:Arterial occlusive disease:0.03), (RALOXIFENE HYDROCHLORIDE:Arterial occlusive disease:0.08), (TERIFLUNOMIDE:Arterial occlusive disease:0.15), (TIZANIDINE HYDROCHLORIDE:Arterial occlusive disease:0.08), (TOPIRAMATE:Arterial occlusive disease:0.9), (TRIAMCINOLONE ACETONIDE:Arterial occlusive disease:0.41), (UMECLIDINIUM BROMIDE\VILANTEROL TRIFENATATE:Arterial occlusive disease:0.16), (ACETAMINOPHEN\BUTALBITAL\CAFFEINE:Arteriosclerosis:0.31), (ALISKI REN HEMIFUMARATE:Arteriosclerosis:0.03), (AMPHETAMINE ASPARTATE\AMPHETAMINE SULFATE\DEXTROAMPHETAMINE SACCHARATE\DEXTROAMPHETAMINE SULFATE:Arteriosclerosis:0.7), (ANASTROZOLE:Arteriosclerosis:1.1),(ARMODAFINIL:Arteriosclerosis: 0.34), (AZITHROMYCIN ANHYDROUS:Arteriosclerosis:0.19), (BENAZEPRIL\BENAZEPRIL HYDROCHLORIDE:Arteriosclerosis:0.09), (BENZONATATE:Arteriosclerosis:0.01), (BISACO DYL OR DOCUSATE SODIUM:Arteriosclerosis:0.8), (BUDESONIDE\FORMOTEROL FUMARATE:Arteriosclerosis:0.61), (BUSPIRONE HYDROCHLORIDE:Arteriosclerosis:0.6), (CALCIUM ACETATE:Arteriosclerosis:0.7), (CALCIUM CARBONATE\CHOLECALCIFEROL:Arteriosclerosis:1.27), (CANAGLIFLOZIN\METFORM IN HYDROCHLORIDE:Arteriosclerosis:0.43), (CARFILZOMIB:Arteriosclerosis:0.36), (CARVEDI LOL PHOSPHATE:Arteriosclerosis:0.74), (CHOLESTYRAMINE:Arteriosclerosis:0.52), (CICLESON IDE:Arteriosclerosis:0.2), (CLOTRIMAZOLE:Arteriosclerosis:0.46), (DANAZOL:Arteriosclerosis: 0.04), (DESLORATADINE:Arteriosclerosis:0.5), (DICLOFENAC:Arteriosclerosis:1.49), (DIG ITOXIN:Arteriosclerosis:0.28), (DIVALPROEX SODIUM:Arteriosclerosis:0.59), (ELETRIPTAN HYDROBROMIDE:Arteriosclerosis:0.26), (EMPAGLIFLOZIN\LINAGLIPTIN:Arteriosclerosis: 0.66), (FLUTICASONE FUROATE:Arteriosclerosis:0.11), (FLUTICASONE FUROATE\SALMETEROL XINAFOATE:Arteriosclerosis:0.41), (FORMOTEROL FUMARATE:Arteriosclerosis:0.45), (GLIMEPIRIDE\ROSIGLITAZONE MALEATE:Arteriosclerosis:0.11), (ILOPROST:Arteriosclerosis:0.43), (IMATINIB MESYLATE:Arteriosclerosis:0.87), (IMIPRAMINE HYDROCHLORIDE:Arteriosclerosis:0.09), (INDAPAMIDE\PERINDOPRIL:Arteriosclerosis:0.66), (IPRATROPIUM:Arteriosclerosis:0.67), (LISDEXAMFETAMINE DIMESYLATE:Arteriosclerosis:0.19), (LITHIUM CARBONATE:Arteriosclerosis:0.77), (LOMITAPIDE MESYLATE:Arteriosclerosis:0.08), (METAXALONE:Arteriosclerosis:0.37), (METFORMIN HYDROCHLORIDE\ROSIGLITAZONE MALEATE:Arteriosclerosis:0.32), (METHADONE HYDROCHLORIDE:Arteriosclerosis:0.45), (METHENAMINE HIPPURATE:Arteriosclerosis:0.16), (METHYLPREDNISOLONE ACETATE:Arteriosclerosis:0.45), (METRONIDAZOLE:Arteriosclerosis:1.3), (NADOLOL:Arteriosclerosis: 0.22), (NATEGLINIDE:Arteriosclerosis:0.31), (NITROFURANTOIN:Arteriosclerosis: 0.57), (NORTRIPTYLINE:Arteriosclerosis:0.33), (OLODATEROL HYDROCHLORIDE\TIOTROPIUM BROMIDE MONOHYDRATE:Arteriosclerosis:0.43), (PAROXETINE\PAROXETINE HYDROCHLORIDE:Arteriosclerosis:0.75), (PATIROMER:Arteriosclerosis:0.03), (PAZOPANI B HYDROCHLORIDE:Arteriosclerosis:0.6), (PEMETREXED DISODIUM:Arteriosclerosis:0.25), (PRAMIPEXOLE:Arteriosclerosis:0.66), (PRAMIPEXOLE DIHYDROCHLORIDE:Arteriosclerosis:0.51), (PROCHLORPERAZINE MALEATE:Arteriosclerosis:0.01), (RISEDRONATE SODIUM:Arteriosclerosis:0.43), (ROFLUMILAST:Arteriosclerosis:0.37), (SCOPOLAMINE:Arteriosclerosis: 0.34), (SECUKINUMAB:Arteriosclerosis:0.31), (SEVELAMER CARBONATE:Arteriosclerosis:0.07), (SEVELAMER HYDROCHLORIDE:Arteriosclerosis:0.13), (SUMATRIPTAN:Arteriosclerosis:0.03), (THALID OMIDE:Arteriosclerosis:0.51), (TIMOLOL:Arteriosclerosis:0.3), (TIOTROPIUM:Arteriosclerosis: 0.42), (VITAMIN B:Arteriosclerosis:0.6), (ZINC:Arteriosclerosis:0.1), (AMITRIPTYLINE:Arteriosclerosis coronary artery:0.09), (CALCIUM ACETATE:Arteriosclerosis coronary artery:0), (CANDESARTAN:Arteriosclerosis coronary artery:0.41), (CANDESARTAN CILEXETIL:Arteriosclerosis coronary artery:0.36), (CYCLOBENZAPRINE:Arteriosclerosis coronary artery:0.02), (DAPAGLIFLOZIN PROPANEDIOL:Arteriosclerosis coronary artery:0.04), (DIPHENHYDRAMINE:Arteriosclerosis coronary artery:0.29), (ENOXAPARIN:Arteriosclerosis coronary artery:0.07), (ERGOCALCIFEROL:Arteriosclerosis coronary artery:0.18), (ESCITALOPRAM OXALATE:Arteriosclerosis coronary artery:0.15), (FLECAINIDE:Arteriosclerosis coronary artery:0.04), (HYDROCHLOROTHIAZIDE\LISINOPRIL:Arteriosclerosis coronary artery:0.03), (METRONIDAZOLE:Arteriosclerosis coronary artery:0.64), (OXYGEN:Arteriosclerosis coronary artery:0.74), (POLYETHYLENE GLYCOLS:Arteriosclerosis coronary artery:0.05), (POTASSIUM:Arteriosclerosis coronary artery:0.7), (TOPIRAMATE:Arteriosclerosis coronary artery:0.27), (VERAPAMIL HYDROCHLORIDE:Arteriosclerosis coronary artery:0.88), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Arthralgia:1.02), (.ALPHA.1-PROTEINASE INHIBITOR HUMAN:Arthralgia:1.69), (ABACAVIR:Arthralgia:0.2), (ABOBOTULINUMTOXINA:Arthralgia: 0.98), (ACAMPROSATE:Arthralgia:0.02), (AGALSIDASE BETA:Arthralgia:2.2), (ALDESLEUKIN:Arthralgia:0.53), (ALISKIREN HEMIFUMARATE\HYDROCHLOROTHIAZIDE:Arthralgia:0.28), (ALOGLIPTIN:Arthralgia: 0.1), (AMEZINIUM METILSULFATE:Arthralgia:0.83), (AMILORIDE HYDROCHLORIDE\FUROSEMIDE:Arthralgia:0.36), (AMINOPHYLLINE:Arthralgia:0.47), (A MINOSALICYLIC ACID:Arthralgia:0.77), (AMLODIPINE BESYLATE\IRBESARTAN:Arthralgia:0.83), (AMMONIUM CHLORIDE\DEXTROMETHORPHAN\SODIUM CITRATE:Arthralgia:0.85), (AMOXAPINE:Arthralgia:0.5), (AMPICILLIN SODIUM:Arthralgia:0.26), (ANAGRELIDE:Arthralgia:0.27), (ARIPIPRAZOLE LAUROXIL:Arthralgia:0.78), (ASCORBIC ACID/CHROMIUM/SELENIUM/VANADYL SULFATE:Arthralgia:0.49), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Arthralgia:0.74), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Arthralgia:0.37), (ASPARAGINASE:Arthralgia: 0.04), (ASPIRIN\CAFFEINE\SALICYLAMIDE:Arthralgia:0.05), (ATORVASTATIN CALCIUM\EZETIMIBE:Arthralgia:0.38), (ATROPINE SULFATE\HYOSCYAMINE SULFATE\PHENOBARBITAL\SCOPOLAMINE HYDROBROMIDE:Arthralgia:0.26), (BALSALAZIDE:Arthralgia:0.65), (BAZEDOXIFENE ACETATE\ESTROGENS, CONJUGATED:Arthralgia:0.36), (BECLOMETHASONE:Arthralgia:1.42), (BENSERAZIDE:Arthralgia: 0.07), (BENZOYL PEROXIDE\CLINDAMYCIN PHOSPHATE:Arthralgia:0), (BEPRIDIL:Arthralgia:1.24), (BETAXOLOL HYDROCHLORIDE:Arthralgia:0.61), (BIFIDOBACTERIUM LONGUM\LACTOBACILLUS ACIDOPHILUS\LACTOBACILLUS RHAMNOSUS:Arthralgia:0.08), (BORON:Arthralgia:0.08), (BOTULINUM TOXIN TYPE A:Arthralgia:1.21), (BRINZOLAMIDE\TIMOLOL MALEATE:Arthralgia:0.53), (BROMHEXINE:Arthralgia:0.19), (BROMOPRIDE:Arthralgia:0.41), (BUTYLSCOPOLAMINE:Arthralgia:0.16), (CABOZANTINIB:Arthralgia:0.31), (CABOZAN TINIB S-MALATE:Arthralgia:0.63), (CALCIUM PANTOTHENATE\CYANOCOBALAMIN\NIACINAMIDE\PYRIDOXINE HYDROCHLORIDE\RIBOFLAVIN\THIAMINE MONONITRATE:Arthralgia:0.9), (CALCIUM\VITAMINS:Arthralgia:0.45), (CANRENOATE POTASSIUM:Arthralgia:0.11), (CARBAZOCHROME SODIUM SULFONATE:Arthralgia:0.48), (CARMUSTINE:Arthralgia:0.06), (CARTEOLOL:Arthralgia:0.15), (CARTEOLOL HYDROCHLORIDE:Arthralgia:0.5), (CEFOTAXIME\CEFOTAXIME SODIUM:Arthralgia:0.96), (CEFPROZIL:Arthralgia:0.41), (CEFTAZIDIME SODIUM:Arthralgia:0.17), (CEPHARANTHINE:Arthralgia:0), (CERIVASTATIN SODIUM:Arthralgia:0.64), (CETIRIZINE HYDROCHLORIDE\PSEUDOEPHEDRINE HYDROCHLORIDE:Arthralgia:1.45), (CETYLPYRIDINIUM CHLORIDE OR SODIUM FLUORIDE OR SODIUM MONOFLUOROPHOSPHATE OR GLUCOSE OXIDASE\LACTOFERRIN\LACTOPEROXIDASE BOVINE\LYSOZYME:Arthralgia:0), (CHLORDIAZEPOXIDE:Arthralgia:0.36), (CHLORMAD INONE ACETATE:Arthralgia:0.5), (CHLOROTHIAZIDE:Arthralgia:0.84), (CHLORPROTHIXENE:Arthralgia: 0.44), (CICLOPIROX OLAMINE:Arthralgia:0.31), (CIFENLINE:Arthralgia:0.27), (CLADRIBINE:Arthralgia:0.04), (C LIKSTAR:Arthralgia:0.78), (CLOBAZAM:Arthralgia:0.6), (CLOFAZIMINE:Arthralgia:0.65), (C LOMIPRAMINE:Arthralgia:0.19), (COBICISTAT\ELVITEGRAVIR\EMTRICITABINE\TENO FOVIR ALAFENAMIDE FUMARATE:Arthralgia:0.3), (COLISTIMETHATE SODIUM:Arthralgia:0.39), (COLISTIN:Arthralgia:0.35), (CYCLOPENTOLATE:Arthralgia:0.24), (DANTROLENE SODIUM:Arthralgia:1.08), (DEFEROXAMINE MESYLATE:Arthralgia:0.36), (DESFLURANE:Arthralgia:0.27), (DIENOGEST:Arthralgia:0.49), (DIETARY SUPPLEMENT\HERBALS\MILK THISTLE:Arthralgia:0.25), (DILAZEP:Arthralgia:0.98), (DIMETHICONE 410:Arthralgia:0.18), (DIPHENIDOL HYDROCHLORIDE:Arthralgia:0.7), (DISOPYRAMIDE:Arthralgia:0.44), (DOLASETRON MESYLATE:Arthralgia:1.18), (DOPAMINE\DOPAMINE HYDROCHLORIDE:Arthralgia:0.17), (DROSPIRENONE\ETHINYL ESTRADIOL\LEVOMEFOLATE CALCIUM:Arthralgia:0.45), (EDARAVONE:Arthralgia:0.63), (EDOXABAN TOSILATE MONOHYDRATE:Arthralgia:0.71), (EDOXABAN TOSYLATE:Arthralgia:3.05), (ELBASVIR\GRAZOPREVIR:Arthralgia:0.53), (EMTRICITABI NE\RILPIVIRINE HYDROCHLORIDE\TENOFOVIR DISOPROXIL FUMARATE:Arthralgia:0.06), (ENFUVIRTIDE:Arthralgia:0.01), (ERIBULIN:Arthralgia:0.87), (ERLOTINIB HYDROCHLORIDE:Arthralgia:0.19), (ERYTHROMYCIN\SULFISOXAZOLE:Arthralgia:0.12), (ESTRADIOL ACETATE:Arthralgia:0.19), (ESTRADIOL BENZOATE:Arthralgia:0.18), (ESTRADIOL\LEVONORGESTREL:Arthralgia:0.26), (ESTROG ENS\METHYLTESTOSTERONE:Arthralgia:0.14), (ETHACRYNIC ACID:Arthralgia:0.92), (ETHINYL ESTRADIOL\NORETHINDRONE:Arthralgia:1.34), (ETHYL ICOSAPENTATE\ICOSAPENT:Arthralgia:0.52), (ETOPOSIDE PHOSPHATE:Arthralgia:0.4), (EUCALYPTOL\MENTHOL\METHYL SALICYLATE\THYMOL:Arthralgia:0.24), (FAROPENEM SODIUM:Arthralgia:0.16), (FELODIPINE\METOPROLOL SUCCINATE:Arthralgia:0.05), (FERRIC CITRATE ANHYDROUS:Arthralgia:0.03), (FERRIC OXYHYDROXIDE:Arthralgia:0.1), (FERROUS BISGLYCINATE:Arthralgia:1.11), (FERROUS SULFATE\GLYCINE:Arthralgia:1.67), (FILGRASTIM-SNDZ:Arthralgia:0.01), (FLAVOXATE HYDROCHLORIDE:Arthralgia:0.3), (FLUNARIZINE HYDROCHLORIDE:Arthralgia:0.62), (FLUOCINOLONE:Arthralgia:0.1), (FLUPHENAZINE: Arthralgia:0.26), (FOSFOMYCIN:Arthralgia:0.5), (FOSPHENYTOIN\FOSPHENYTOIN SODIUM:Arthralgia:0.48), (GIMERACIL\OTERACIL\TEGAFUR:Arthralgia:1.06), (GLIMEPI RIDE\ROSIGLITAZONE MALEATE:Arthralgia:0.02), (GUANABENZ ACETATE:Arthralgia:0.06), (GUANFACINE HYDROCHLORIDE:Arthralgia:0.66), (HALCINONIDE:Arthralgia:0.12), (HALOBETASOL PROPIONATE:Arthralgia:0.36), (HOMATROPINE METHYLBROMIDE\HYDROCODONE BITARTRATE:Arthralgia:0.02), (HORSE CHESTNUT:Arthralgia:0.56), (HYDRAZINE SULFATE:Arthralgia:0.06), (HYDROCHLOROTHIAZIDE\METOPROLOL SUCCINATE:Arthralgia:0.57), (HYDROQUINONE:Arthralgia:0.11), (HYDROXYCITRIC ACID:Arthralgia:0.2), (IBRITUMOMAB TIUXETAN:Arthralgia:0.92), (ICOSAPENT:Arthralgia:0.7), (IDARUCIZUMAB:Arthralgia:0.31), (IFENPRODIL:Arthralgia:0.05), (ILOPROST:Arthralgia:1.52), (IMIGLUCERASE:Arthralgia:10.49), (INDACATEROL MALEATE:Arthralgia:0.06), (INSULIN PORK\ANSULIN PURIFIED PORK:Arthralgia:0.45), (INTERFERON:Arthralgia:2.35), (INTERFERON ALFA-2A:Arthralgia:1.82), (INTERFERON ALFA-2A OR INTERFERON ALFA-2B:Arthralgia:0.23), (INTERFERON GAMMA-1B:Arthralgia:0.61), (IPRAGLIFLOZIN L-PROLINE:Arthralgia:0.08), (IRON\VITAMINS:Arthralgia:0.41), (ITOPRIDE:Arthralgia:0.02), (I TRACONAZOLE\SODIUM CHLORIDE:Arthralgia:0.42), (IVABRADINE HYDROCHLORIDE:Arthralgia:0.43), (IVACAFTOR\LUMACAFTOR:Arthralgia:0.19), (KALL IDINOGENASE:Arthralgia:0.2), (LEVOCARNITINE HYDROCHLORIDE:Arthralgia:0.24), (LEVOLEUCOVORIN CALCIUM:Arthralgia:0.29), (LINSIDOMINE:Arthralgia:0.51), (LUTETIUM LU-177: Arthralgia:0.72), (LYSOZYME HYDROCHLORIDE:Arthralgia:0.3), (MAGNESIUM ASPARTATE\POTASSIUM ASPARTATE:Arthralgia:0.45), (MAGNESIUM PIDOLATE:Arthralgia:0.2), (MAPROTILINE HYDROCHLORIDE:Arthralgia:0.36), (MECLIZINE MONOHYDROCHLORIDE\NIACIN:Arthralgia:0.17), (MELPERONE:Arthralgia:1.01), (MEPO LIZUMAB:Arthralgia:1.56), (MESTRANOL\NORETHINDRONE:Arthralgia:0.08), (METFOR MIN HYDROCHLORIDE\ROSIGLITAZONE MALEATE:Arthralgia:0.15), (METFORMIN HYDROCHLORIDE\SAXAGLIPTIN:Arthralgia:0.5), (METHENOLONE ENANTHATE:Arthralgia:0.96), (METHYLNALTREXONE BROMIDE:Arthralgia:0.2), (METRELEPTIN:Arthralgia:0.7), (MEXILETINE:Arthralgia:0.56), (MIGLUSTAT:Arthralgia:0.38), (MILRINONE:Arthralgia:0.26), (MITIGLINIDE CALCIUM HYDRATE\VOGLIBOSE:Arthralgia:0.3), (NABILONE:Arthralgia:0.22), (NALOXONE:Arthralgia: 0.8), (NEFAZODONE:Arthralgia:0.23), (NEFAZODONE HYDROCHLORIDE:Arthralgia:0.47), (NIMODIPINE:Arthralgia:0.24), (NISOLDIPINE:Arthralgia: 0.57), (NOREPINEPHRINE BITARTRATE:Arthralgia:1.12), (OLODATEROL HYDROCHLORIDE:Arthralgia:0.12), (OXYBUTYNIN\OXYBUTYNIN CHLORIDE:Arthralgia:1.26), (PANITUMUMAB:Arthralgia:0.49), (PARATHYROID HORMONE:Arthralgia:0.5), (PARICALCITOL:Arthralgia:0.92), (PEGINTERFERON ALFA-2A OR PEGINTERFERON ALFA-2B:Arthralgia:0.36), (PEGINTERFERON ALFA-2A OR PEGINTERFERON ALFA-2B OR PEGINTERFERON BETA-1A OR PEGINTERFERON LAMBDA-1A:Arthralgia:0.01), (PEGZEREPOETIN ALFA:Arthralgia:0.32), (PENICILLAMINE:Arthralgia:0.41), (PENTAMIDINE:Arthralgia:0.42), (PENTOSTATIN:Arthralgia:0.71), (PERINDOPRIL ARGININE:Arthralgia:0.19), (PHENYTOIN SODIUM:Arthralgia:2.19), (PIMOBENDAN:Arthralgia:1.06), (PINDOLOL:Arthralgia:0.36), (PI PERACILLIN:Arthralgia:0.5), (PIRITRAMIDE:Arthralgia:0.72), (PITOFENONE:Arthralgia:0.06), (POLYVINYL ALCOHOL:Arthralgia:0), (PORFIMER SODIUM:Arthralgia:0.03), (POTASSIUM PHOSPHATE, MONOBASIC:Arthralgia:0.15), (POVIDONE:Arthralgia:0.65), (PRAMLINTIDE ACETATE:Arthralgia:0.32), (PRAMOXINE HYDROCHLORIDE:Arthralgia:0.5), (PROCYCLIDINE:Arthralgia:0.06), (PROTRIPTYLINE HYDROCHLORIDE:Arthralgia:1.41), (PRUCALOPRIDE SUCCINATE:Arthralgia:0.44), (PSORALEN:Arthralgia:0.1), (PYRIDOSTIGMINE:Arthralgia:0.2), (QUERCETIN:Arthralgia:0.07), (QUINACRINE:Arthralgia:1.96), (QUINIDINE GLUCONATE:Arthralgia:0.02), (RAMUCIRUMAB:Arthralgia:0.93), (RILMENIDINE:Arthralgia: 0.27), (RILPIVIRINE:Arthralgia:0.01), (RILPIVIRINE HYDROCHLORIDE:Arthralgia:0.9), (ROPIVACAINE HYDROCHLORIDE:Arthralgia:0.07), (RUPATADINE FUMARATE:Arthralgia:0.13), (SELEGILINE:Arthralgia:1.35), (SEVELAMER:Arthralgia:0.29), (SIBUTRAMINE HYDROCHLORIDE:Arthralgia:0.93), (SODIUM CITRATE:Arthralgia:0.51), (SORBITOL:Arthralgia:0.57), (STREPTOMYCIN\STREPTOMYCIN SULFATE:Arthralgia:0.34), (SUFENTANIL:Arthralgia:0.41), (SUFENTANIL CITRATE:Arthralgia:0.74), (SULFACETAMIDE SODIUM\SULFUR:Arthralgia:0.38), (SULPIRIDE:Arthralgia:0.58), (SUPLATAST TOSILATE:Arthralgia:0.83), (TAURINE:Arthralgia:0.21), (TAZOBACTAM:Arthralgia:0.95), (T EDUGLUTIDE:Arthralgia:0.51), (TEGASEROD MALEATE:Arthralgia:0.22), (TELOTRISTAT ETHYL:Arthralgia:0.61), (TEMOCAPRIL HYDROCHLORIDE:Arthralgia:0.25), (TEMSIROLIMUS:Arthralgia:0.38), (TIMOLOL MALEATE\TRAVOPROST:Arthralgia:1.42), (TIMOLOL\TRAVOPROST:Arthralgia:0.43), (TO REMIFENE CITRATE:Arthralgia:0.04), (TREPROSTINIL DIOLAMINE:Arthralgia:0.13), (TRIMEBUTINE:Arthralgia:0.25), (TRIMIPRAMINE MALEATE:Arthralgia:1.69), (TRYPTOPHAN:Arthralgia:0.05), (UBIQUINOL:Arthralgia:0.4), (VASOPRESSIN:Arthralgia:0.34), (VELAGLUCERASE ALFA:Arthralgia:0.1), (VINPOCETINE:Arthralgia:0.02), (ZILEUTON:Arthralgia:1.23), (ZIPRA SIDONE HYDROCHLORIDE:Arthralgia:0.02), (ZIV-AFLIBERCEPT:Arthralgia:0.11), (.ALPHA.1-PROTEINASE INHIBITOR HUMAN:Arthritis:1.93), (.BETA.-CAROTENE:Arthritis:0.03), (ABOBOTULINUMTOXINA:Arthritis:0.87), (ACEBUTOLOL HYDROCHLORIDE:Arthritis:0.69), (ACETAMINOPHEN\DICHLORALPHENAZONE\ISOM ETHEPTENE:Arthritis:0.62), (ACETAMINOPHEN\METHOCARBAMOL:Arthritis:1.24), (AC TIVATED CHARCOAL:Arthritis:0.58), (AMILORIDE HYDROCHLORIDE\FUROSEMIDE:Arthritis:0.54), (AMINO ACIDS\DEXTROSE\ELECTROLYTES NOS:Arthritis:1.38), (AMINOPHYLLINE:Arthritis:0.21), (AMLODIPINE BESYLATE\IRBESARTAN:Arthritis:0.07), (AMMONIUM CHLORIDE\DEXTROMETHORPHAN\SODIUM CITRATE:Arthritis:0.71), (ANAGRELIDE HYDROCHLORIDE:Arthritis:0.74), (ANTIHEMOPHILIC FACTOR, HUMAN RECOMBINANT RESIDUES 743-1636 DELETED:Arthritis:0.1), (ARIPIPRAZOLE LAUROXIL:Arthritis:0.92), (ASPIRIN\CAFFEINE\SALICYLAMIDE:Arthritis:0.62), (ATROPI NE SULFATE:Arthritis:0.4), (AZITHROMYCIN MONOHYDRATE:Arthritis:0.56), (BALSALAZIDE DISODIUM:Arthritis:1.53), (BECLOMETHASONE DIPROPIONATE\FORMOTEROL:Arthritis:0.15), (BELATACEPT:Arthritis:0.01), (BENZOCA INE:Arthritis:0.31), (BENZOYL PEROXIDE\CLINDAMYCIN PHOSPHATE:Arthritis:0.19), (BEPRIDIL:Arthritis:0.09), (BETAMETHASONE DIPROPIONATE\CALCIPOTRIENE HYDRATE:Arthritis:0.86), (BICARBONATE ION:Arthritis:0.05), (BIFIDOBACTERIUM SPP.:Arthritis:0.19), (BIMATOPROST\TIMOLOL:Arthritis:0.3), (BIPERIDEN HYDROCHLORIDE:Arthritis:0.04), (BLINATUMOMAB:Arthritis:0.4), (BORON:Arthritis:0.14), (CABOZANTINIB S-MALATE:Arthritis:0.64), (CAJUPUT OIL\EUCALYPTUS OIL\PEPPERMINT OIL:Arthritis:0.15), (CAMPHOR\DIPHENHYDRAMINE\ZINC OXIDE:Arthritis:0.2), (CARBIMAZOLE:Arthritis:0.92), (CARBOMER:Arthritis:0.74), (CARTE OLOL HYDROCHLORIDE:Arthritis:0.43), (CEFOTAXIME SODIUM:Arthritis:1.39), (CEFPODOXIME PROXETIL:Arthritis:0.77), (CEFPROZIL:Arthritis:0.4), (CEFTAZIDIME:Arthritis:0.4), (CERITI NIB:Arthritis:0.31), (CERIVASTATIN SODIUM:Arthritis:1.02), (CERTOPARIN SODIUM:Arthritis:0.22), (CHLORAMPHENICOL:Arthritis:0.1), (CHLORMADINONE ACETATE\ETHINYL ESTRADIOL:Arthritis:0.03), (CHOLECALCIFEROL\GENISTEIN\ZINC GLYCINATE:Arthritis:0.05), (CICLOPIROX OLAMINE:Arthritis:0.42), (CILASTATIN SODIUM\IMIPENEM:Arthritis:1.37), (CLIKSTAR:Arthritis:0.9), (CLONIXIN\CYCLOBENZA PRINE:Arthritis:0.03), (CODEINE PHOSPHATE\PROMETHAZINE:Arthritis:0.2), (CODEINE\PROMETHAZINE:Arthritis:0.13), (COUMARIN\TROXERUTIN:Arthritis:0.08), (DABRAFENIB MESYLATE:Arthritis:0.26), (DACLATASVIR:Arthritis:0.63), (DANTROLENE SODIUM:Arthritis:0.81), (DEGARELIX:Arthritis:0.17), (DEXAMETHASONE ACETATE:Arthritis:1.25), (DEXAMETHASONE SODIUM PHOSPHATE:Arthritis:1.05), (DEXMETHYLPHENIDATE HYDROCHLORIDE:Arthritis:0.28), (DIENOGEST:Arthritis:0.15), (DIGITOXIN:Arthritis:1.12), (DIMETHICONE 410:Arthritis:0.38), (DOBUTAMINE HYDROCHLORIDE:Arthritis:0.04), (DOLASETRON MESYLATE:Arthritis:0.9), (DOXORUBICIN HYDROCHLORIDE:Arthritis:0.05), (DOXYLAMINE SUCCINATE\PYRIDOXINE HYDROCHLORIDE:Arthritis:0.14), (DROSPIRENONE\ETHINYL ESTRADIOL\LEVOMEFOLATE CALCIUM:Arthritis:0.28), (ELOTUZUMAB:Arthritis:0.94), (ENSULIZOLE\EUCERIN\OCTIN OXATE\OCTISALATE\TITANIUM DIOXIDE\ZINC OXIDE:Arthritis:0.3), (EPHEDRINE HYDROCHLORIDE\GUAIFENESIN:Arthritis:0.22), (ERIBULIN:Arthritis:0.28), (ERTAPENE M SODIUM:Arthritis:0.3), (ESMOLOL HYDROCHLORIDE:Arthritis:0.02), (ESTRADIOL ACETATE:Arthritis:0.21), (ESTRADIOL BENZOATE:Arthritis:0.22), (ESTROGENS, ESTERIFIED:Arthritis:0.19), (ESTROGENS\METHYLTESTOSTERONE:Arthritis:0.19), (ETHI NYL ESTRADIOL\FERROUS FUMARATE\NORETHINDRONE:Arthritis:0), (ETHINYL ESTRADIOL\FERROUS FUMARATE\NORETHINDRONE ACETATE:Arthritis:0.2), (ETHINYL ESTRADIOL\NORGESTREL:Arthritis:1.08), (ETRETINATE:Arthritis:1.2), (FERRIC POLYSACCHARIDE COMPLEX:Arthritis:0.22), (FESOTERODINE:Arthritis:0.05), (FLUOCINOLONE ACETONIDE:Arthritis:1.31), (FORMOTEROL FUMARATE\MOMETASONE FUROATE:Arthritis:1.98), (GIMERACIL\OTERACIL\TEGAFUR:Arthritis:0.31), (GLYCOPYR RONIUM\INDACATEROL:Arthritis:2.41), (GUAIFENESIN\PHENYLEPHRINE HYDROCHLORIDE:Arthritis:0.02), (HALCINONIDE:Arthritis:0.16), (HEPARIN CALCIUM:Arthritis:0.75), (HERBALS\MINERALS\VITAMINS:Arthritis:0.38), (HORSE CHESTNUT:Arthritis:0.85), (HYDROQUINONE:Arthritis:0.48), (IMIGLUCERASE:Arthritis:0.98), (INSULIN PORK\INSULIN PURIFIED PORK:Arthritis:0.55), (INTERFERON ALFA-2A:Arthritis:1.16), (INTERFERON GAMMA-1B:Arthritis:0.77), (IRINOTECAN HYDROCHLORIDE:Arthritis:0.68), (IRON SUCROSE:Arthritis:1.65), (IRON\POLYSACCHARIDES:Arthritis:0.69), (ITRACONAZOLE\SODIUM CHLORIDE:Arthritis:0.23), (KETOTIFEN:Arthritis:0.04), (LENVATINIB:Arthritis:0.44), (LEV ODOPA:Arthritis:0.32), (LEVOLEUCOVORIN CALCIUM:Arthritis:0.02), (LEVOMEFOLIC ACID\METHYLCOBALAMIN\TURMERIC:Arthritis:0.43), (LEVOTHYROXINE SODIUM\POTASSIUM IODIDE:Arthritis:1.01), (LUMACAFTOR:Arthritis:0.03), (MECLIZINE MONOHYDROCHLORIDE\NIACIN:Arthritis:0.32), (MELPERONE:Arthritis:0.14), (MENAQ UINONE 6:Arthritis:0.35), (MENTHOL\ZINC OXIDE:Arthritis:0.43), (MESNA:Arthritis:0.47), (MESTRANOL\NORETHINDRONE:Arthritis: 0.02), (METFORMIN HYDROCHLORIDE\SAXAGLIPTIN:Arthritis:0.06), (METFORMIN PAMOATE:Arthritis:0.09), (METHENAMINE MANDELATE:Arthritis:0.94), (METHENOLONE ENANTHATE:Arthritis:0.13), (METHOXY POLYETHYLENE GLYCOL-EPOETIN BETA:Arthritis:0.22), (METRELEPTIN:Arthritis:0.56), (NADROPARIN CALCIUM:Arthritis:1.49), (NALOXONE:Arthritis:0.31), (NALTREXONE HYDROCHLORIDE:Arthritis:0.32), (NEOMYCIN:Arthritis:0.88), (NIMODIPINE:Arthritis:0.1), (NIRAPARIB:Arthritis:1.21), (NOREPINEPHRINE:Arthritis:0.03), (OCTREOTIDE:Arthritis:1.23), (OLODATEROL HYDROCHLORIDE:Arthritis:0.13), (OSIMERTINIB:Arthritis:0.04), (OXITRIPTAN:Arthritis:0.04), (PALONOSETRON HYDROCHLORIDE:Arthritis:0.84), (PANITUMUMAB:Arthritis:0.34), (PAPAYA:Arthritis:0.32), (PAZOPANIB HYDROCHLORIDE:Arthritis:2.99), (PERMETHRIN:Arthritis:0.39), (PHENYLEPHRINE HYDROCHLORIDE\PROMETHAZINE HYDROCHLORIDE:Arthritis:0.06), (POLYETHYLENE GLYCOL 3350\POTASSIUM CHLORIDE\SODIUM BICARBONATE\SODIUM CHLORIDE\SODIUM SULFATE ANHYDROUS:Arthritis:0.87), (POLYETHYLENE GLYCOL 4000:Arthritis:0.74), (POLYVINYL ALCOHOL:Arthritis:0.09), (POVIDONE:Arthritis:0.41), (PRANLUKAST:Arthritis:0.48), (PRED NISOLONE SODIUM PHOSPHATE:Arthritis:0.62), (PREDNISOLONE SODIUM SUCCINATE:Arthritis:0.12), (PREGNENOLONE:Arthritis:0.61), (PROCATEROL HYDROCHLORIDE:Arthritis:0.33), (PRUCALOPRIDE SUCCINATE:Arthritis:0.45), (PSORALEN:Arthritis:0.12), (PYRIDOSTIGMINE:Arthritis:0.08), (QUINIDINE GLUCONATE:Arthritis:0.21), (RADIATION THERAPY:Arthritis:1.21), (RAMUCIRUMAB:Arthritis:0.32), (RILPIVIRINE HYDROCHLORIDE:Arthritis:0.16), (RUCAPARIB CAMSYLATE:Arthritis:0.11), (SELEXIPAG:Arthritis:0.97), (SEVELAMER HYDROCHLORIDE:Arthritis:0.95), (SHARK LIVER OIL:Arthritis:0.05), (SILICON DIOXIDE:Arthritis:0.06), (SUFENTANIL CITRATE:Arthritis:0.01), (SULFACETAMIDE SODIUM\SULFUR:Arthritis:0.41), (SUPLATAST TOSILATE:Arthritis:0.2), (TAZAROTENE:Arthritis:0.23), (TAZOBACTAM:Arthritis:0.19), (TE A LEAF:Arthritis:0.16), (TELOTRISTAT ETHYL:Arthritis:0.84), (TEMOZOLOMIDE:Arthritis:0.36), (TESAMORELIN:Arthritis:0.68), (T HYMOL:Arthritis:0.01), (TIAGABINE HYDROCHLORIDE:Arthritis:0.03), (TICARCILLIN:Arthritis:0.11), (TILIDINE:Arthritis:0.51), (TIMOLOL MALEATE\TRAVOPROST:Arthritis:1.34), (TIMOLOL\TRAVOPROST:Arthritis:0.38), (TINZ APARIN:Arthritis:0.69), (TRAMETINIB DIMETHYL SULFOXIDE:Arthritis:0.29), (TRIMETAZIDINE DIHYDROCHLORIDE:Arthritis:0.18), (TRIMIPRAMINE MALEATE:Arthritis:1.01), (TRIPTORELIN PAMOATE:Arthritis:1.06), (TRYPTOPHAN:Arthritis:0.05), (UBIQUINOL:Arthritis:0.67), (VA NDETANIB:Arthritis:0.19), (VINORELBINE TARTRATE:Arthritis:0.68), (WARFARIN POTASSIUM:Arthritis:0.56), (ZILEUTON:Arthritis:1.45), (ACARBOSE:Arthropathy:0.15), (AC ETAZOLAMIDE:Arthropathy:0.56), (ADO-TRASTUZUMAB EMTANSINE:Arthropathy:0.05), (ALCOHOL:Arthropathy:0.16), (AMANTADINE:Arthropathy: 0.68), (AMIODARONE:Arthropathy:1.23), (AMLODIPINE BESYLATE\HYDROCHLOROTHIAZIDE\OLMESARTAN MEDOXOMIL:Arthropathy:0.05), (BETAMETHASONE\CALCIPOTRIENE:Arthropathy:0.03), (BISACODYL:Arthropathy:0.5), (BORTEZOMIB:Arthropathy:0.22), (BUDESONIDE\FORMO TEROL FUMARATE:Arthropathy:0.33), (BUPRENORPHINE HYDROCHLORIDE\NALOXONE HYDROCHLORIDE:Arthropathy:0.84), (CALCIUM\MAGNESIUM\ZINC:Arthropathy:0.65), (CANAGLIFLOZIN\METFORMIN HYDROCHLORIDE:Arthropathy:0.39), (CANAKINUMAB:Arthropathy:0.3), (CANDESARTA N CILEXETIL:Arthropathy:1.36), (CANDESARTAN CILEXETIL\HYDROCHLOROTHIAZIDE:Arthropathy:1.09), (CAPTOPRIL:Arthropathy:0.45), (CARFILZOMIB:Arthropathy:0.41), (CARVEDILOL PHOSPHATE:Arthropathy:1.17), (CICLESONIDE:Arthropathy:0.2), (CLINDAMYCIN:Arthropathy: 0.69), (CLINDAMYCIN PHOSPHATE:Arthropathy:0.1), (CLOZAPINE:Arthropathy:0.44), (CODEINE:Arthropathy:0.51), (DACLIZUMAB:Arthropathy:0.62), (DASATINIB:Arthropathy:1.25), (DESONIDE:Arthropathy: 0.43), (DRONABINOL:Arthropathy:0.84), (EMPAGLIFLOZIN\LINAGLIPTIN:Arthropathy:0.71), (ESTROGENS:Arthropathy:0.46), (FLUDROCORTISONE ACETATE:Arthropathy:0.38), (FLUTICASONE FUROATE:Arthropathy:0.34), (FLUTICASONE FUROATE\SALMETEROL XINAFOATE:Arthropathy:0.29), (FLUTICASONE FUROATE\VILANTEROL TRIFENATATE:Arthropathy:1.03), (FLUVOXAMINE MALEATE:Arthropathy:0.12), (FORMOTEROL FUMARATE DIHYDRATE\MOMETASONE FUROATE:Arthropathy:0.94), (FUSIDIC ACID:Arthropathy:0.17), (GLYBURIDE:Arthropathy:0.84), (GLYCOPYRRONIUM\INDACAT EROL:Arthropathy:0.27), (HEPARIN SODIUM:Arthropathy:0.2), (HYDROCODONE BITARTRATE\IBUPROFEN:Arthropathy:0.34), (HYDROCORTISONE ACETATE:Arthropathy:0.06), (HYDROMORPHONE:Arthropathy:0.94), (ILOPROST:Arthropathy: 0.06), (IMATINIB MESYLATE:Arthropathy:0.93), (INDAPAMIDE\PERINDOPRIL:Arthropathy:0.17), (INSULIN DEGLUDEC:Arthropathy:0.15), (LACTASE:Arthropathy:0.4), (LEDIPASVIR\SOFOSBUVIR: Arthropathy:0.57), (LIDOCAINE\PRILOCAINE:Arthropathy:0.16), (LOMITAPIDE MESYLATE:Arthropathy:0.51), (METHENAMINE HIPPURATE:Arthropathy:0.76), (MIDODRINE:Arthropathy:0.19), (MINOCYCLINE\MINOCY CLINE HYDROCHLORIDE:Arthropathy:0.8), (NADOLOL:Arthropathy:0.58), (NATEGLINIDE:Arthropathy: 0.2), (NORETHINDRONE:Arthropathy:0.34), (OXYBUTYNIN\OXYBUTYNIN CHLORIDE:Arthropathy:0.23), (PAMIDRONATE DISODIUM:Arthropathy:0.39), (PANTOPRAZOLE MAGNESIUM:Arthropathy:1.2), (PAROXETINE\PAROXETINE HYDROCHLORIDE:Arthropathy:1.07), (PENICILLIN:Arthropathy:0.97), (PONATINIB:Arthropathy: 0.42), (PRAZOSIN HYDROCHLORIDE:Arthropathy:0.17), (PROPAFENONE HYDROCHLORIDE:Arthropathy:0.4), (PROTRIPTYLINE HYDROCHLORIDE:Arthropathy:0.19), (QUINAPRIL\QUINAPRIL HYDROCHLORIDE:Arthropathy:0.09), (RIVASTIGMINE:Arthropathy:0.32), (ROFLUMILAS T:Arthropathy:0.39), (SCOPOLAMINE:Arthropathy:0.59), (SULFAMETHOXAZOLE:Arthropathy: 0.14), (SUMATRIPTAN:Arthropathy:0.89), (TERBINAFINE HYDROCHLORIDE:Arthropathy:0.72), (TERBUTALINE SULFATE:Arthropathy:0.42), (TETRACYCLINE:Arthropathy:0.86), (TIMOLOL MALEATE:Arthropathy:0.28), (TIOTROPIUM:Arthropathy:0.15), (TOLTERODINE:Arthropathy: 0.08), (UMECLIDINIUM BROMIDE\VILANTEROL TRIFENATATE:Arthropathy:0.76), (CERTOLIZUMAB PEGOL:Arthropod bite:0.03), (METRONIDAZOLE:Arthropod bite:0.08), (ACETAMINOPHEN\OXYCODONE HYDROCHLORIDE:Ascites:0), (ALCOHOL:Ascites:0.2), (ASCORBIC ACID:Ascites:0.02), (BROMAZEPAM:Ascites:0.52), (BUPRENORPHINE HYDROCHLORIDE\NALOXONE HYDROCHLORIDE:Ascites:0.11), (BUPROPION:Ascites:0.02), (CERTOLIZUMAB PEGOL:Ascites:1.3), (CLOTRIMAZOLE:Ascites:0.33), (CRIZOTINIB:Ascites:0.62), (DESLOR ATADINE:Ascites:0.27), (DEVICE:Ascites:0.14), (DICLOFENAC:Ascites:0.79), (DIVALPROE X SODIUM:Ascites:0.06), (DOXAZOSIN\DOXAZOSIN MESYLATE:Ascites:0.35), (DOXYCYCLINE:Ascites:0.05), (EDOXABAN TOSYLATE:Ascites:0.15), (EPINEPHRINE:Ascites:0.39), (EVOLOCUMAB:Ascites:0.49), (EX EMESTANE:Ascites:0.5), (FLECAINIDE:Ascites:0.15), (HYDROCHLOROTHIAZIDE\LISINO PRIL:Ascites:0.48), (ILOPROST:Ascites:0.42), (INDAPAMIDE:Ascites:0.15), (INDAPAMIDE\P ERINDOPRIL:Ascites:0.54), (INTERFERON:Ascites:0.05), (ISOSORBIDE:Ascites:0.19), (LEV OLEUCOVORIN CALCIUM:Ascites:0.75), (METOLAZONE:Ascites:0.18), (NILOTINIB:Ascites:1.64), (OLAPA RIB:Ascites:0.02), (PANTOPRAZOLE MAGNESIUM:Ascites:0.23), (PAROXETINE:Ascites:0.15), (PAROXETINE\PAROXETINE HYDROCHLORIDE:Ascites:0.01), (PEMETREXED:Ascites:0.23), (PERTUZUMAB:Ascites:0.06), (PITAVASTATIN CALCIUM:Ascites:0.24), (RANOLAZINE:Ascites:0.22), (ROFLUMILAST:Ascites:0.08), (SEC UKINUMAB:Ascites:0.54), (SELEXIPAG:Ascites:0.56), (SOTALOL:Ascites:0.51), (SULFAME THOXAZOLE:Ascites:0.04), (TETRACYCLINE:Ascites:0.08), (TOPIRAMATE:Ascites:0.39), (VALSARTAN:Ascites:1.36), (.ALPHA.1-PROTEINASE INHIBITOR HUMAN:Aspergillus infection:0.44), (ALBUMIN HUMAN:Aspergillus infection:0.21), (ANASTROZOLE:Aspergillus infection:0.16), (BECLOMETHASONE DIPROPIONATE:Aspergillus infection:0.4), (BELATACEPT:Aspergillus infection:0.04), (BENDAMUSTINE:Aspergillus infection:0.39), (BORTEZOMIB:Aspergillus infection:0.18), (BRINCIDOFOVIR:Aspergillus infection:0.02), (BROMAZEPAM:Aspergillus infection:0.16), (BUPROPION HYDROCHLORIDE:Aspergillus infection:0.48), (CALCITRIOL:Aspergillus infection:0.5), (CANAKINUMAB:Aspergillus infection:0.14), (CANDESARTAN:Aspergillus infection:0.42), (CANDESARTAN CILEXETIL\HYDROCHLOROTHIAZIDE:Aspergillus infection:0.49), (CEFOXITIN\CEFOXITIN SODIUM:Aspergillus infection:0.11), (CEFUROXIME:Aspergillus infection:0.32), (CELECOXIB:Aspergillus infection:0.73), (CERTOLIZUMAB PEGOL:Aspergillus infection:0.94), (CHLORAMBUCIL:Aspergillus infection:0.23), (CICLESONIDE:Aspergillus infection:0.52), (CLINDAMYCIN\CLINDAMYCIN PHOSPHATE:Aspergillus infection:1.46), (CLONIDINE:Aspergillus infection:0.2), (COLCHICINE:Aspergillus infection:0.44), (COLISTIMETHATE SODIUM:Aspergillus infection:1.04), (CYCLOBENZAPRINE:Aspergillus infection:0.11), (CYPROHEPTADINE:Aspergillus infection:0.19), (DEXLANSOPRAZOLE:Aspergillus infection:0.65), (DICLOFENAC SODIUM:Aspergillus infection:0.61), (DIVALPROEX SODIUM:Aspergillus infection:0.43), (ECONAZOLE:Aspergillus infection:0.33), (ELOTUZUMAB:Aspergillus infection:0.07), (ERYTHROMYCIN:Aspergillus infection:0.21), (ESZOPICLONE:Aspergillus infection:0.26), (ETOPOSIDE PHOSPHATE:Aspergillus infection:0.05), (EVOLOCUMAB:Aspergillus infection:0.08), (FENOTEROL HYDROBROMIDE\IPRATROPIUM BROMIDE:Aspergillus infection:0.15), (FLUTICASONE:Aspergillus infection:0.38), (FORMOTEROL FUMARATE DIHYDRATE\MOMETASONE FUROATE:Aspergillus infection:0.17), (FORMOTEROL FUMARATE\MOMETASONE FUROATE:Aspergillus infection:0.59), (GEMTUZUMAB OZOGAMICIN:Aspergillus infection:0.03), (GLYCOPYRROLATE:Aspergillus infection:0.09), (GLYCOPYRRONIUM:Aspergillus infection:0.42), (IBRITUMOMAB TIUXETAN:Aspergillus infection:0.27), (ILOPROST:Aspergillus infection:0.34), (IMATINIB MESYLATE:Aspergillus infection:0.39), (INDAPAMIDE\PERINDOPRIL:Aspergillus infection:0.57), (INFLUENZA VIRUS VACCINE:Aspergillus infection:0.06), (IRBESARTAN:Aspergillus infection:0.3), (IVACAFTOR\LUMACAFTOR:Aspergillus infection:0.29), (LABETALOL\LABETALOL HYDROCHLORIDE:Aspergillus infection:0.19), (LEDIPASVIR\SOFOSBUVIR:Aspergillus infection:0.02), (LENALIDOMIDE:Aspergillus infection:0.45), (LERCANIDIPINE HYDROCHLORIDE:Aspergillus infection:0.03), (LUMACAFTOR:Aspergillus infection:0.23), (MELOXICAM:Aspergillus infection:0.46), (MEPOLIZUMAB:Aspergillus infection:0.38), (METHYLPREDNISOLONE ACETATE:Aspergillus infection:0.19), (NILOTINIB:Aspergillus infection:0.48), (OLOPATADINE:Aspergillus infection:0.32), (OXCARBAZEPINE:Aspergillus infection:1.09), (PANTOPRAZOLE MAGNESIUM:Aspergillus infection:0.99), (PENTOSTATIN:Aspergillus infection:0.54), (PRAMIPEXOLE DIHYDROCHLORIDE:Aspergillus infection:0.04), (PRAVASTATIN:Aspergillus infection:0.69), (PREDNISOLONE SODIUM PHOSPHATE:Aspergillus infection:0.25), (PRIMIDONE:Aspergillus infection:0.32), (RANOLAZINE:Aspergillus infection:0.27), (RISEDRONATE SODIUM:Aspergillus infection:0.51), (SULFAMETHOXAZOLE:Aspergillus infection:0.36), (TETRACYCLINE:Aspergillus infection:0.09), (THALIDOMIDE:Aspergillus infection:0.08), (TOFACITINIB CITRATE:Aspergillus infection:0.03), (ABIRATERONE ACETATE:Asthenia:0.24), (ACARBOSE:Asthenia:0.3), (ACETAMINOPHEN\OXYCODONE HYDROCHLORIDE\OXYCODONE TEREPHTHALATE:Asthenia:0.28), (ACETAMINOPHEN\OXYCODONE\OXYCODONE HYDROCHLORIDE:Asthenia:0.5), (ACETAZOLAMIDE:Asthenia:0.41), (ALBIGLUTIDE:Asthenia: 0.15), (ALENDRONIC ACID:Asthenia:0.2), (ALFACALCIDOL:Asthenia:0.1), (ALISKIREN HEMIFUMARATE:Asthenia:0.08), (ALUMINUM HYDROXIDE\DIMETHICONE\MAGNESIUM HYDROXIDE:Asthenia:0.01), (BUDESONIDE\FORMOTEROL FUMARATE:Asthenia:0.13), (CALCIUM CARBONATE\VITAMIN D:Asthenia:0.89), (CALCIUM\MAGNESIUM:Asthenia:0.21), (CANAGLIFLOZIN\METFORMI N HYDROCHLORIDE:Asthenia:0.27), (CANAKINUMAB:Asthenia:0.15), (CANDESARTAN:Asthenia: 1.38), (CANDESARTAN CILEXETIL:Asthenia:0.96), (CARVEDILOL PHOSPHATE:Asthenia:0.93), (CETIRIZINE HYDROCHLORIDE\PSEUDOEPHEDRINE HYDROCHLORIDE:Asthenia:0.23), (CHLORDIAZEPOXIDE HYDROCHLORIDE\CLIDINIUM BROMIDE:Asthenia:0.07), (CILOSTAZOL:Asthenia:0.05), (CLINDAMYCIN\CLINDAMYCIN PHOSPHATE:Asthenia:1.36), (CLONIDINE HYDROCHLORIDE:Asthenia:0.03), (COD LIVER OIL:Asthenia:0.26), (COLCHICINE:Asthenia:1.03), (COLESEVELAM HYDROCHLORIDE:Asthenia:0.38), (DABIGATRAN:Asthenia:0.21), (DALTEPARIN:Asthenia: 0.2), (DALTEPARIN SODIUM:Asthenia:0.19), (DASATINIB:Asthenia:1.12), (DEFLAZACORT:Asthenia:0.04), (DIM ENHYDRINATE:Asthenia:0.53), (DRONABINOL:Asthenia:0.92), (EDOXABAN TOSYLATE:Asthenia:0.22), (EMPAGLIFLOZIN\LINAGLIPTIN:Asthenia:0.39), (ETORICOXI B:Asthenia:0.17), (EVOLOCUMAB:Asthenia:1.35), (FEXOFENADINE\PSEUDOEPHEDRINE: Asthenia:0.05), (FLUDROCORTISONE ACETATE:Asthenia:0.28), (FLUOCINONIDE:Asthenia:0.4), (FLUTICASONE FUROATE:Asthenia:0.37), (FLUTICASONE FUROATE\SALMETEROL XINAFOATE:Asthenia:0.25), (FORMOTEROL:Asthenia:0.02), (FORMOTEROL FUMARATE:Asthenia:0.16), (FUSIDIC ACID:Asthenia:0.25), (GADOLINIUM:Asthenia:0.39), (GLYCOPYRRONIUM\INDACATERO L:Asthenia:0.16), (HYDROCHLOROTHIAZIDE\TELMISARTAN:Asthenia:0.32), (HYDROCH LOROTHIAZIDE\VALSARTAN:Asthenia:1.07), (HYDROCODONE BITARTRATE\IBUPROFEN:Asthenia:0.04), (IMATINIB MESYLATE:Asthenia:1.2), (IMIPRAMINE HYDROCHLORIDE:Asthenia:0.61), (INDAPAMIDE\PERINDOPRIL:Asthenia:0.47), (INDOM ETHACIN:Asthenia:0.45), (INFLIXIMAB-DYYB:Asthenia:0.11), (INSULIN GLULISINE:Asthenia:0.28), (LANREOTIDE ACETATE:Asthenia:0.4), (LERCANIDIPINE:Asthenia:0.64), (LERCANIDIPINE HYDROCHLORIDE:Asthenia:0.68), (LIDOCAINE HYDROCHLORIDE:Asthenia:0.84), (LITHIUM CARBONATE:Asthenia:1.44), (LOMITAPIDE MESYLATE:Asthenia:0.37), (METHYLPREDNISOLONE ACETATE:Asthenia:0.55), (METRONIDAZOLE\METRONIDAZOLE HYDROCHLORIDE:Asthenia:1.01), (MUPIROCIN\MUPIROCIN CALCIUM:Asthenia:0.14), (NALTREXONE:Asthenia:0.26), (NATEGLINIDE:Asthenia:0.13), (NILOTINIB:Asthenia:1.4), (NIRAPARIB:Asthenia:0.07), (NORETHINDRONE:Asthenia:0.23), (NORTRIPTYLINE HYDROCHLORIDE:Asthenia:0.14), (OXAZEPAM:Asthenia:0.38), (PANTOPRAZOLE MAGNESIUM:Asthenia:0.92), (PAZOPANIB HYDROCHLORIDE:Asthenia:0.34), (PONATINIB:Asthenia:0.79), (PRAZOSIN:Asthenia:0.32), (PRAZOSIN HYDROCHLORIDE:Asthenia:0.14), (PROPAFENONE HYDROCHLORIDE:Asthenia:0.1), (RABEPRAZOLE:Asthenia:0.64), (RIZATRIPTAN BENZOATE:Asthenia:1.02), (SECUKINUMAB:Asthenia:0.82), (TERBINAFINE HYDROCHLORIDE:Asthenia:0.31), (TETRACYCLINE:Asthenia:0.84), (THALIDOMIDE:Asthenia: 0.88), (TIOTROPIUM:Asthenia:0.3), (TOCILIZUMAB:Asthenia:0.52), (TOCOPHEROL:Asthenia: 0.09), (UMECLIDINIUM BROMIDE\VILANTEROL TRIFENATATE:Asthenia:0.87), (VANCOMYCIN HYDROCHLORIDE:Asthenia:0), (VILAZODONE HYDROCHLORIDE:Asthenia:0.5), (ZONISAMIDE:Asthenia:0.25), (.ALPHA.-TOCOPHEROL\RETINOL:Asthma:0.96), (.BETA.-HYDROXYISOVALERIC ACID\ARGININE\GLUTAMINE:Asthma:0.45), (. GAMMA.-AMINOBUTYRIC ACID:Asthma:0.04), (ACEMETACIN:Asthma:0.08), (ACETAMINOPHEN\BROMISOVAL\DI HYDROCODEINE PHOSPHATE\DIPHENHYDRAMINE SALICYLATE\DYPHYLLINE\METHYLEPHEDRINE HYDROCHLORIDE, (+/−)-:Asthma:0.31), (ACETAMINOPHEN\CAFFEINE\CARISOPRODOL\DICLOFENAC SODIUM:Asthma:0.72), (ACETAMINOPHEN\CAFFEINE\OPIUM:Asthma:1.64), (ACETAMI NOPHEN\CHLORPHENIRAMINE MALEATE\PSEUDOEPHEDRINE HYDROCHLORIDE:Asthma:0.1), (ACETAMINOPHEN\DICHLORALPHENAZONE\ISOME THEPTENE HYDROCHLORIDE:Asthma:0.26), (ACETAMINOPHEN\GUAIFENESIN\PHENYLEPHRINE HYDROCHLORIDE:Asthma:0.02), (ACETAMINOPHEN\METHOCARBAMOL:Asthma:1.29), (ACETAMINOPHEN\PSEUDOEPHEDRINE HYDROCHLORIDE:Asthma:0.09), (ACYCLOVIR SODIUM:Asthma:0.12), (ALBUMIN BOVINE\TANNIC ACID:Asthma:0.82), (ALEFACEPT:Asthma:0.02), (ALFALFA:Asthma:0.66), (ALISKIREN HEMIFUMARATE\HYDROCHLOROTHIAZIDE:Asthma:0.43), (ALISKIREN HEMIFUMARATE\VALSARTAN:Asthma:0.53), (ALLANTOIN\BENZOCAINE\CAMPHOR (NATURAL)\DIMETHICONE\MENTHOL:Asthma:0.15), (ALOSETRON HYDROCHLORIDE:Asthma:0.04), (ALUMINUM HYDROXIDE\MAGNESIUM HYDROXIDE\SIMETHICONE:Asthma:0.86), (ALUMINUM HYDROXIDE\MAGNESIUM OXIDE\OXETHAZAINE:Asthma:0.3), (ALUMINUM SILICATE:Asthma:0.42), (AMILORIDE HYDROCHLORIDE:Asthma:0.21), (AMINO ACIDS\CARBOHYDRATES\ELECTROLYTES NOS\VITAMINS:Asthma:0.54), (AMINO ACIDS\CARBOHYDRATES\MINERALS\SOYBEAN OIL\VITAMINS:Asthma:0.29), (AMINOBENZOIC ACID\CALCIUM PANTOTHENATE\CYSTINE\KERATIN\THIAMINE MONONITRATE:Asthma:0.48), (AMITRIPTYLINE\CHLORDIAZEPOXIDE:Asthma:0.06), (A MLODIPINE\LOSARTAN:Asthma:0.25), (AMOXICILLIN SODIUM:Asthma:0.48), (AMOXICILLIN\CLARITHROMYCIN\LANSOPRAZOLE:Asthma:0.56), (ANIDULAFUNGIN:Asthma:0.22), (APRINDINE HYDROCHLORIDE:Asthma:0.83), (ARCTOSTAPHYLOS UVA-URSI LEAF\URTICA DIOICA:Asthma:0.48), (ASCORBIC ACID\BIOTIN\CALCIUM PANTOTHENATE\CYANOCOBALAMIN\FOLIC ACID\NIACINAMIDE\PYRIDOXINE HYDROCHLORIDE\RIBOFLAVIN\THIAMINE MONONITRATE:Asthma:0.66), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Asthma:1.24), (ASCORBIC ACID\FERROUS SULFATE\FOLIC ACID\PYRIDOXINE HYDROCHLORIDE\RIBOFLAVIN\THIAMINE MONONITRATE:Asthma:0.89), (ASCORBIC ACID\POLYETHYLENE GLYCOL 3350\POTASSIUM CHLORIDE\SODIUM ASCORBATE\SODIUM CHLORIDE\SODIUM SULFATE:Asthma:0.6), (ASCORBIC ACID\VITAMIN B COMPLEX:Asthma:0.34), (ASENAPINE:Asthma:0.23), (ASPARAGINASE:Asthma:0.43), (AT ORVASTATIN CALCIUM\EZETIMIBE:Asthma:0.43), (ATROPINE SULFATE\DIPHENOXYLATE:Asthma:0.21), (AVANAFIL:Asthma:0.37), (AVOCADO OIL\SOYBEAN OIL:Asthma:0.47), (AZITHROMYCIN OR METHOTREXATE SODIUM:Asthma:1.58), (BACITRACIN ZINC\POLYMYXIN B SULFATE:Asthma:0.43), (BACITRACIN\NEOMYCIN SULFATE:Asthma:0.17), (BACITRACIN\NEOMYCIN SULFATE\POLYMYXIN B SULFATE:Asthma:0.05), (BACLOFEN\CYCLOBENZAPRINE\DICLOFENAC\GABAPENTI N\TETRACAINE:Asthma:0.31), (BELATACEPT:Asthma:0.39), (BENIDIPINE:Asthma:0.33), (B ENPROPERINE PHOSPHATE:Asthma:0.02), (BENZOCAINE\RESORCINOL:Asthma:0.4), (BERAPROST:Asthma: 1.77), (BETA GLUCAN:Asthma:0.1), (BETAMETHASONE DIPROPIONATE\CALCIPOTRIENE HYDRATE:Asthma:0.47), (BIFIDOBACTERIUM LONGUM:Asthma:0.26), (BIFIDOBACTERIUM LONGUM\LACTOBACILLUS ACIDOPHILUS\LACTOBACILLUS RHAMNOSUS:Asthma:0.56), (BIPERIDEN LACTATE:Asthma:0.07), (BLINATUMOMAB:Asthma:1.3), (BORIC ACID:Asthma:0.53), (BORON:Asthma:0.37), (BOSUTINIB:Asthma:0.35), (BOTULISM ANTITOXIN:Asthma:0.49), (BROMPERIDOL:Asthma:0.16), (BUFORMIN:Asthma:0.17), (BUT ORPHANOL:Asthma:0.77), (CABOZANTINIB:Asthma:0.2), (CAFFEINE\ISOMETHEPTENE\METHIMAZOLE:Asthma:0.7), (CAFFEINE\MAGNESIUM SALICYLATE:Asthma:0.14), (CALCIUM CHLORIDE\DEXTROSE\POTASSIUM CHLORIDE\SODIUM ACETATE\SODIUM CHLORIDE:Asthma:0.66), (CALCIUM GLYCEROPHOSPHATE\POLYETHYLENE GLYCOLS\POWDERED CELLULOSE\SACCHARIN SODIUM DIHYDRATE\SODIUM HYDROXIDE\SODIUM LAURYL SULFATE\SODIUM MAGNESIUM SILICATE\SODIUM MONOFLUOROPHOSPHATE\SORBITOL\WATER:Asthma:0.32), (CALCIUM LACTATE:Asthma:1.63), (CALCIUM PANTOTHENATE\CYANOCOBALAMIN\NIACINAMIDE\PYRIDOXINE HYDROCHLORIDE\RIBOFLAVIN\THIAMINE MONONITRATE:Asthma:1.81), (CAMPHOR (SYNTHETIC)\LEVOMENTHOL\MENTHYL SALICYLATE OR CAMPHOR (SYNTHETIC)\MENTHOL\MENTHYL SALICYLATE OR MENTHOL\MENTHYL SALICYLATE:Asthma:0.23), (CAPSICUM:Asthma:0.11), (CARBOHYDRATES\FATTY ACIDS\MINERALS\PROTEIN\VITAMINS:Asthma:0.09), (CARMUSTINE:Asthma:0.06), (CA RTEOLOL HYDROCHLORIDE:Asthma:0.85), (CEFCAPENE PIVOXIL HYDROCHLORIDE:Asthma:0.88), (CEFOTAXIME\CEFOTAXIME SODIUM:Asthma:1.18), (CEFTERAM PIVOXIL:Asthma:1.07), (CERTOLIZUMAB:Asthma:0.23), (CHAMOMILE:Asthma:0.18), (CH LORMADINONE ACETATE\ETHINYL ESTRADIOL:Asthma:0.58), (CHLORMADINONE ACETATE\MESTRANOL:Asthma:0.01), (CHLORMEZANONE:Asthma:0.08), (CHLOROQUI NE PHOSPHATE:Asthma:0.57), (CHLORPHENIRAMINE MALEATE\HYDROCODONE BITARTRATE\PSEUDOEPHEDRINE HYDROCHLORIDE:Asthma:0.06), (CHLORPROMAZINE\PROMETHAZINE:Asthma:0.38), (CHOLINE\TRAZODONE HYDROCHLORIDE:Asthma:0.45), (CILAZAPRIL ANHYDROUS:Asthma:0.33), (CIPROFLOXACIN\DEXAMETHASONE:Asthma:0.08), (CLIK STAR:Asthma:1.05), (CLOCORTOLONE PIVALATE:Asthma:0.95), (CLOMETHIAZOLE:Asthma:0.16), (CLONIXIN\CYCLOBENZAP RINE:Asthma:0.07), (CLOXACILLIN SODIUM:Asthma:0.44), (CODEINE PHOSPHATE\GUAIFENESIN\PSEUDOEPHEDRINE HYDROCHLORIDE:Asthma:0.18), (COLESTILAN CHLORIDE:Asthma:0.77), (CONVALLARIA MAJALIS\CRATAEGUS LAEVIGATA LEAF\PROXYPHYLLINE:Asthma:1.21), (CRANBERRY CONCENTRATE:Asthma:0.59), (CREATINE MONOHYDRATE:Asthma:0.37), (CYANOCOBALAMIN CO-57\FOLIC ACID\PYRIDOXINE HYDROCHLORIDE:Asthma:0.23), (CYANOCOBALAMIN\DICLOFENAC SODIUM\PYRIDOXINE HYDROCHLORIDE\THIAMINE MONONITRATE:Asthma:0.86), (CYANOCOBALAMIN\FOLIC ACID\PYRIDOXINE HYDROCHLORIDE:Asthma:0.3), (CYCLOPENTOLATE:Asthma:0.21), (CYSTEAMINE BITARTRATE:Asthma:0), (CYSTEINE:Asthma:0.65), (DABRAFENIB MESYLATE:Asthma:0.56), (DACLATASVIR DIHYDROCHLORIDE:Asthma:0.33), (DANTROLENE:Asthma:0.21), (DARIFENACIN:Asthma: 0.11), (DEFERIPRONE:Asthma:1.09), (DEOXYRIBONUCLIEC ACID:Asthma:0.96), (DESVENLAFAXINE:Asthma:1.06), (DEXIBUPROFEN:Asthma:0.07), (D EXTROMETHORPHAN HYDROBROMIDE\GUAIFENESIN\PSEUDOEPHEDRINE HYDROCHLORIDE:Asthma:0.06), (DIACEREIN:Asthma:0.1), (DIASTASE:Asthma:0.04), (DI CHLORPHENAMIDE:Asthma:0.15), (DICLOFENAC DIETHYLAMINE:Asthma:0.47), (DIENOGEST:Asthma:1.82), (DIETARY SUPPLEMENT\RESVERATROL:Asthma:1), (DIFLUCORTOLONE VALERATE\LIDOCAINE:Asthma:0.18), (DIFLUNISAL:Asthma:1.77), (DIGITALIS:Asthma:0.54), (DIGITOXIN:Asthma:1.82), (DIPHENHYDRAMINE HYDROCHLORIDE\DOXYLAMINE SUCCINATE:Asthma:0.4), (DIPHTHERIA AND TETANUS TOXOIDS AND ACELLULAR PERTUSSIS VACCINE ADSORBED:Asthma:0.53), (DISTIGMINE BROMIDE:Asthma:1), (DOCUSATE SODIUM\SENNOSIDE A\SENNOSIDE B:Asthma:0.42), (DOLASETRON MESYLATE:Asthma:1.65), (DORIPENEM:Asthma:0), (DOXERCALCIFEROL:Asthma:0.09), (DOXYCYCLINE HYDROCHLORIDE:Asthma:0.64), (EFLORNITHINE HYDROCHLORIDE:Asthma:0.39), (ELOTUZUMAB:Asthma:2.01), (EMTRICITABINE\RILPI VIRINE\TENOFOVIR DISOPROXIL FUMARATE:Asthma:0.42), (EPHEDRINE HYDROCHLORIDE:Asthma:0.85), (ERGOLOID MESYLATES:Asthma:0.44), (ERIBULIN:Asthma:0.78), (ERIBULIN MESYLATE:Asthma:0.41), (ERTAPENEM:Asthma:0.34), (ERTAPENEM SODIUM:Asthma:0.99), (ERYTHROMYCIN\SULFISOXAZOLE:Asthma:0), (ESTRADIOL BENZOATE:Asthma:1.06), (ESTRADIOL VALERATE:Asthma:0.77), (ESTROGENS\METHYLTESTOSTERONE:Asthma:0.54), (ESTRO NE:Asthma:0.24), (ETEPLIRSEN:Asthma:0.34), (ETHINYL ESTRADIOL\ETHYNODIOL:Asthma:0.35), (ETHINYL ESTRADIOL\FERROUS FUMARATE\NORETHINDRONE:Asthma:0.97), (ETHYL ICOSAPENTATE\ICOSAPENT:Asthma:1.01), (ETILEFRINE HYDROCHLORIDE:Asthma:0.38), (ETOPOSIDE PHOSPHATE:Asthma:0.03), (ETRETINATE:Asthma:2.37), (FAROPENEM SODIUM:Asthma:0.95), (FERRIC OXYHYDROXIDE:Asthma:1.13), (FERRIC POLYSACCHARIDE COMPLEX:Asthma:0.84), (FERROUS BISGLYCINATE:Asthma:1.74), (FERROUS SULFATE\GLYCINE:Asthma:2.39), (FILGRASTIM-SNDZ:Asthma:0.29), (FINGOLIMOD:Asthma:0.2), (FLAVOXATE HYDROCHLORIDE:Asthma:0.74), (FLUPENTIXOL:Asthma:0.98), (FLUPENTIXOL DIHYDROCHLORIDE:Asthma:0.01), (FLUPHENAZINE DECANOATE:Asthma:0.23), (FLUTAMIDE:Asthma:0.96), (FOLIC ACID\IRON POLYMALTOSE:Asthma:0.06), (FOLLICLE STIMULATING HORMONE BETA POLYPEPTIDE\LUTROPIN ALFA:Asthma:0.09), (FOSAPREPITANT DIMEGLUMINE:Asthma:0.18), (FOSFOMYCIN:Asthma:1.02), (FOSFOMYCIN CALCIUM:Asthma:1), (FOSINOPRIL SODIUM\HYDROCHLOROTHIAZIDE:Asthma:0.16), (FUROSEMIDE\SPIRONOLACTONE: Asthma:0.3), (FURSULTIAMINE:Asthma:0.88), (GABEXATE MESYLATE:Asthma:0.18), (GADOBUTROL:Asthma:0.59), (GADOTERATE MEGLUMINE:Asthma:10.2), (GLIMEPIRIDE\METFORMIN:Asthma:0.35), (GLUCONATE:Asthma: 0.89), (GOLD:Asthma:0.03), (GUANABENZ ACETATE:Asthma:0.84), (HALOPERIDOL DECANOATE:Asthma:1.11), (HEPATITIS B VIRUS VACCINE:Asthma:0.98), (HERBALS\MINERALS\VITAMINS:Asthma:0.96), (HERBS\ROOT S:Asthma:0.16), (HORSE CHESTNUT:Asthma:1.3), (HYDROCHLORIC ACID:Asthma:0.23), (HYDROCHLOROTHIAZIDE\METOPROLOL SUCCINATE:Asthma:0.72), (HYDROCHLOROTHIAZIDE\METOPROLOL TARTRATE:Asthma:0.62), (HYDROCORTISONE PROBUTATE:Asthma:0.98), (HYDROCORTISONE VALERATE:Asthma:0.52), (HYDROXOCOBALAMIN ACETATE:Asthma:0.27), (HYPERICUM PERFORATUM:Asthma:0.06), (IBRITUMOMAB TIUXETAN:Asthma:0.53), (IFENPRODIL:Asthma:0.8), (IMIGLUCERASE:Asthma:2.19), (IMIP RAMINE PAMOATE:Asthma:0.42), (INCOBOTULINUMTOXINA:Asthma:0.13), (INFLUENZA VIRUS VACCINE TRIVALENT TYPE A+B AFLURIA:Asthma:0.72), (INSULIN ASPART\INSULIN DEGLUDEC:Asthma:0.28), (INSULIN PORK\INSULIN PURIFIED PORK:Asthma:0.98), (INTERFERON ALFA-2A OR INTERFERON ALFA-2B:Asthma:0.45), (INTERFERON ALFA-2B:Asthma:1.35), (IODINE\POTASSIUM IODIDE:Asthma:0.47), (IRINOTECAN HYDROCHLORIDE:Asthma:1.4), (ISOFLAVONES SOY:Asthma:0.35), (ISONIAZID\PYRAZINAMIDE\RIFAMPIN:Asthma:0.48), (KALLIDINOG ENASE:Asthma:0.5), (LACIDIPINE:Asthma:0.58), (LACTOPEROXIDASE BOVINE\LYSOZYME HYDROCHLORIDE:Asthma:0.03), (LANDIOLOL HYDROCHLORIDE:Asthma:0.49), (LANOLIN\MINERAL OIL\PETROLATUM:Asthma:0.33), (LATANOPROST\TIMOLOL:Asthma:0.37), (LETROZOL E\RIBOCICLIB:Asthma:0.01), (LEVAMLODIPINE:Asthma:0.1), (LEVAMLODIPINE BESYLATE:Asthma:0.13), (LEVOBUNOLOL:Asthma:0.04), (LEVOBUNOLOL HYDROCHLORIDE:Asthma:0.07), (LEVOMEFOLATE CALCIUM\METHYLCOBALAMIN\PYRIDOXAL 5-PHOSPHATE:Asthma:0.18), (LEVOMEFOLIC ACID\METHYLCOBALAMIN\TURMERIC:Asthma:0.02), (LEVOMEPROMAZINE MALEATE:Asthma:0.22), (LEVOSIMENDAN:Asthma:0.17), (LINSIDOMINE:Asthma:0.73), (L OMUSTINE:Asthma:0.02), (LOVASTATIN\NIACIN:Asthma:0.45), (LUMACAFTOR:Asthma: 2.06), (LUTETIUM LU-177:Asthma:1.18), (LYCOPENE:Asthma:0.04), (MAGALDRATE:Asthma:0.65), (MAGNESIUM CARBONATE:Asthma:0.91), (MAGNESIUM PIDOLATE:Asthma:0.41), (MAGNESIUM SULFATE\POTASSIUM SULFATE\SODIUM SULFATE:Asthma:0.06), (MAGNESIUM\PYRIDOXINE:Asthma:0.85), (MAPROTILINE HYDROCHLORIDE:Asthma:0.88), (MATRICARIA RECUTITA:Asthma:0.17), (MECLIZINE MONOHYDROCHLORIDE\NIACIN:Asthma:0.6), (MECLOZINE DIHYDROCHLORIDE:Asthma:0.58), (MENTHOL\ZINC OXIDE:Asthma:1.23), (MEPENZOLATE BROMIDE:Asthma:0.18), (MEPIVACAINE HYDROCHLORIDE:Asthma:0.31), (METAMIZOLE MAGNESIUM:Asthma:0.14), (METFORMIN HYDROCHLORIDE\SAXAGLIPTIN:Asthma:0.73), (METHENAMINE MANDELATE:Asthma:1.2), (METHENOLONE ENANTHATE:Asthma:2.09), (METHSUXIMIDE:Asthma:0.09), (METHYLENE BLUE:Asthma:0.52), (METYRAPONE:Asthma:0.37), (MILNACIPRAN:Asthma:0.48), (MILRIN ONE LACTATE:Asthma:0.32), (MIPOMERSEN SODIUM:Asthma:0.1), (MITIGLINIDE CALCIUM HYDRATE\VOGLIBOSE:Asthma:0.7), (MITOXANTRONE HYDROCHLORIDE:Asthma:0.51), (MORPHINE\MORPHINE HYDROCHLORIDE\MORPHINE SULFATE\MORPHINE TARTRATE:Asthma:0.53), (NAFAMOSTAT MESYLATE:Asthma:0.08), (NALBUPHINE HYDROCHLORIDE:Asthma:1.17), (NEOSTIGMINE METHYLSULFATE:Asthma:0.37), (NUSINERSEN:Asthma:1.97), (OBETICHOLIC ACID:Asthma:0.2), (OXANDROLONE:Asthma:0.69), (PAPAYA:Asthma:0.62), (PENICILLIN V BENZATHINE:Asthma:0.45), (PENTAERYTHRITOL TETRANITRATE:Asthma:0.16), (PENTOSTATIN:Asthma:0.77), (PERMETHRIN:Asthma:0.37), (PEROSPIRONE:Asthma:0.53), (PHENELZINE:Asthma:0.33), (PHENELZINE SULFATE:Asthma:0.58), (PHENOBARBITAL SODIUM:Asthma:1.25), (PHENYLEPHRINE HYDROCHLORIDE\PROMETHAZINE HYDROCHLORIDE:Asthma:0.77), (PHENYLEPHRINE HYDROCHLORIDE\WITCH HAZEL:Asthma:1.28), (PIMOBENDAN:Asthma:1.71), (PITOFENONE:Asthma:0.39), (PLANT AGO OVATA LEAF:Asthma:0.04), (POLYETHYLENE GLYCOL 3350\POTASSIUM CHLORIDE\SODIUM BICARBONATE\SODIUM CHLORIDE\SODIUM SULFATE ANHYDROUS:Asthma:1.71), (POLYMYXIN B SULFATE\TRIMETHOPRIM SULFATE:Asthma:0.56), (POLYSTYRENE:Asthma:0.35), (POLYSTYRENE SULFONIC ACID:Asthma:0.28), (POLYVINYL ALCOHOL\POVIDONE:Asthma:0.62), (PORFIMER SODIUM:Asthma:0.61), (POTASSIUM BITARTRATE:Asthma:0.11), (POTASSIUM BROMIDE:Asthma:0.26), (POTASSIUM CHLORIDE\SODIUM CHLORIDE:Asthma:0.07), (POTASSIUM CHLORIDE\SODIUM CHLORIDE\SODIUM LACTATE:Asthma:0.74), (POTASSIUM CITRATE\SODIUM CITRATE\ZINC:Asthma:0.43), (POTASSIUM PHOSPHATE, MONOBASIC:Asthma:0.64), (POTASSIUM PHOSPHATE\SODIUM PHOSPHATE:Asthma:0.22), (PREDNISOLONE ACETATE\SULFACETAMIDE SODIUM:Asthma:0.55), (PREDNISOLONE SODIUM METAZOATE:Asthma:0.04), (PROCAINE HYDROCHLORIDE:Asthma:0.1), (PROMAZINE:Asthma:0.3), (PROMAZINE HYDROCHLORIDE:Asthma:0.45), (PROPIVERINE:Asthma:0.87), (PROPOXYPHENE:Asthm a:0.1), (PSORALEN:Asthma:0.57), (PYRIMETHAMINE:Asthma:0.72), (QUERCUS SALICINA STEM:Asthma:0.09), (QUINACRINE:Asthma:2.46), (QUINAGOLIDE HYDROCHLORIDE:Asthma:0.99), (QUINIDINE GLUCONATE:Asthma:0.13), (RAMUCIRUMAB:Asthma:1.12), (RANIMUSTINE:Asthma:0.04), (RASAGILINE:Asthma:0.48), (RASPBERRY KETONE:Asthma:0.22), (RED CLOVER:Asthma:0.11), (REGADENOSON:Asthma:0.01), (RHUBARB:Asthma:1.13), (RILME NIDINE:Asthma:0.47), (RILPIVIRINE:Asthma:0.12), (ROPIVACAINE:Asthma:0.71), (RUCAP ARIB CAMSYLATE:Asthma:0.14), (RUFINAMIDE:Asthma:0.52), (SARGRAMOSTIM:Asthma:0.12), (SARPOGRELATE HYDROCHLORIDE:Asthma:0.81), (SEBELIPASE ALFA:Asthma:0.13), (SENNOSIDE B:Asthma:0.32), (SHARK CARTILAGE\SHARK, UNSPECIFIED:Asthma:0.14), (SIVELESTAT SODIUM:Asthma:0.16), (SODIUM BICARBONATE\SODIUM PHOSPHATE, MONOBASIC:Asthma:0.01), (SODIUM CITRATE:Asthma:1.36), (SODIUM FLUORIDE\VITAMINS:Asthma:0.18), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Asthma:0.96), (SODIUM PHOSPHATE, MONOBASIC, ANHYDROUS:Asthma:0.2), (SPIRAMYCIN:Asthma:0.11), (STREPTOMYCIN SULFATE:Asthma:0.12), (SUCCINYLCHOLINE CHLORIDE:Asthma:1.36), (SUFENTANIL CITRATE:Asthma:1.85), (SULBUTIAMINE:Asthma:0.06), (TABALUMAB:Asthma:0.58), (TAF AMIDIS MEGLUMINE:Asthma:0.36), (TALIGLUCERASE ALFA:Asthma:0.31), (TAURINE:Asthma:1.19), (TAZOBACTAM SODIUM:Asthma:0.02), (TBO-FILGRASTIM:Asthma:0.37), (TEGASEROD:Asthma:0.41), (TELOTRISTAT ETHYL:Asthma:0.7), (TEMOCAPRIL:Asthma:0.21), (TETRACAINE HYDROCHLORIDE:Asthma:0.36), (THIOPENTAL SODIUM:Asthma:0.26), (THIOTHIXENE HYDROCHLORIDE:Asthma:0.05), (THROMBOMODULIN ALFA:Asthma:0.24), (THYMOL:Asthma:0.27), (TIANEPTINE SODIUM:Asthma:0.11), (TIAPRIDE:Asthma:0.13), (TILIDINE HYDROCHLORIDE:Asthma:0.08), (TIMOLOL\TRAVOPROST:Asthma:0.95), (TIOPRONIN: Asthma:0.45), (TOFACITINIB:Asthma:0.66), (TOLMETIN:Asthma:0.03), (TOLMETIN SODIUM:Asthma:0.05), (TOPIROXOSTAT:Asthma:0.12), (TOREMIFENE CITRATE:Asthma:1.2), (TRAMETINIB DIMETHYL SULFOXIDE:Asthma:0.62), (TREPROSTINIL DIOLAMINE:Asthma:1.2), (TRIMIPRAMINE MALEATE:Asthma:2.11), (TRIPTORELIN:Asthma:0.16), (TRIPTORELIN PAMOATE:Asthma:1.44), (ULIPRISTAL:Asthma:0.23), (UROKINASE:Asthma:0.65), (VELAG LUCERASE ALFA:Asthma:0.69), (VINBLASTINE SULFATE:Asthma:0.09), (VITAMIN A PALMITATE:Asthma:0.51), (VORAPAXAR SULFATE:Asthma:0.04), (WARFARIN POTASSIUM:Asthma:2.29), (WHEY:Asthma:0.08), (ZALTOPROFEN:Asthma:0.65), (ZICONO TIDE ACETATE:Asthma:1.67), (ZIV-AFLIBERCEPT:Asthma:0.19), (ZUCLOPENTHIXOL DECANOATE:Asthma:0.7), (BRIVARACETAM:Astrocytoma, low grade:0.18), (BRIVARACETAM:Atonic seizures:0.02), (ABOBOTULINUMTOXINA:Atrial fibrillation:1.08), (ACETAMINOPHEN\APRONALIDE\CAFFEINE\PROPYPHENAZONE:Atri al fibrillation:1.3), (ACETAMINOPHEN\BROMISOVAL\DIHYDROCODEINE PHOSPHATE\DIPHENHYDRAMINE SALICYLATE\DYPHYLLINE\METHYLEPHEDRINE HYDROCHLORIDE, (+/−)-:Atrial fibrillation:0.28), (ACETAMINOPHEN\CAFFEINE\CARISOPRODOL\DICLOFENAC:Atrial fibrillation:0.45), (ACETAMINOPHEN\CHLORPHENIRAMINE MALEATE\PSEUDOEPHEDRINE HYDROCHLORIDE:Atrial fibrillation:0.71), (ACETAMINOPHEN\CHLORZOXAZONE:Atrial fibrillation:0.55), (ACETAMINOPHEN\METHOCARBAMOL:Atrial fibrillation:0.84), (ACETAMINOPHEN\OPIUM:Atrial fibrillation:0.17), (ACETAMINOPHEN\PSEUDOEPHEDRINE HYDROCHLORIDE:Atrial fibrillation:0.12), (ACETIC ACID\SODIUM ACETATE:Atrial fibrillation:0.58), (ACETYLCYSTEINE\BENZALKONIUM CHLORIDE\TUAMINOHEPTANE SULFATE:Atrial fibrillation:0.21), (ADENINE:Atrial fibrillation:1.46), (ADUCANUMAB:Atrial fibrillation:0.74), (ALBUTEROL\GUAIFENESIN:Atrial fibrillation:0.79), (ALCLOMETASONE DIPROPIONATE:Atrial fibrillation:0.38), (ALDESLEUKIN:Atrial fibrillation:0.66), (ALDIOXA:Atrial fibrillation:0.52), (ALECTINIB:Atrial fibrillation:0.55), (ALECTINIB HYDROCHLORIDE:Atrial fibrillation:0.25), (ALFALFA:Atrial fibrillation:0.06), (ALGELDRATE:Atrial fibrillation:0.07), (ALISKIREN\HYDROCHLOROTHIAZIDE:Atrial fibrillation:0.62), (ALPROSTADIL ALFADEX:Atrial fibrillation:0.32), (ALUMINUM HYDROXIDE\DIMETHICONE\DIPHENHYDRAMINE\HYDROXIDE ION\LIDOCAINE:Atrial fibrillation:0.13), (ALUMINUM HYDROXIDE\MAGNESIUM OXIDE\OXETHAZAINE:Atrial fibrillation:1.32), (ALUMINUM SILICATE:Atrial fibrillation:0.84), (AMANTADINE SULFATE:Atrial fibrillation:0.61), (AMINO ACIDS\VITAMINS:Atrial fibrillation:0.35), (AMINOCAPROIC ACID:Atrial fibrillation:0.56), (AMINOSALICYLIC ACID:Atrial fibrillation:0.32), (AMLODIPINE\LOSARTAN:Atrial fibrillation:0.58), (AMMONIUM GLYCYRRHIZATE\CYSTEINE HYDROCHLORIDE\GLYCINE:Atrial fibrillation:0.38), (AMOBARBITAL\BELLADONNA LEAF\DIMETHICONE\ERGOTAMINE TARTRATE:Atrial fibrillation:0.1), (AMOXAPINE:Atrial fibrillation:1.71), (AMOXICILLIN\CLARITHROMYCIN\ESOMEPRAZOLE:Atrial fibrillation:2.09), (AMPICILLIN SODIUM:Atrial fibrillation:1.19), (ANGIOTENSIN II:Atrial fibrillation:0.38), (ANTIHISTAMINES NOS:Atrial fibrillation:1.49), (APOMORPHINE:Atrial fibrillation:0.22), (APPLE CIDER VINEGAR:Atrial fibrillation:0.24), (ARSENIC TRIOXIDE:Atrial fibrillation:0.03), (ASCORBIC ACID\BIOTIN\CALCIUM PANTOTHENATE\CYANOCOBALAMIN\FOLIC ACID\NIACINAMIDE\PYRIDOXINE HYDROCHLORIDE\RIBOFLAVIN\THIAMINE HYDROCHLORIDE:Atrial fibrillation:0.37), (ASCORBIC ACID\FERROUS SULFATE\FOLIC ACID\PYRIDOXINE HYDROCHLORIDE\RIBOFLAVIN\THIAMINE MONONITRATE:Atrial fibrillation:0.32), (ASENAPINE:Atrial fibrillation:0.28), (ASPARAGINASE:Atrial fibrillation:0.14), (ASPARTIC ACID:Atrial fibrillation:0.12), (ASPERGILLUS NIGER.ALPHA.-GALACTOSIDASE:Atrial fibrillation:0.45), (ASPIRIN\BUTALBITAL\CAFFEINE\CODEINE PHOSPHATE:Atrial fibrillation:0.06), (ASPIRIN\CAFFEINE\SALICYLAMIDE:Atrial fibrillation:0.53), (ASPIRIN\DIHYDROXYALUMINUM AMINOACETATE\MAGNESIUM CARBONATE:Atrial fibrillation:0.41), (ATRACURIUM:Atrial fibrillation:0.79), (ATROPINE SULFATE\HYOSCYAMINE SULFATE\PHENOBARBITAL\SCOPOLAMINE HYDROBROMIDE:Atrial fibrillation:0.44), (AZASETRON HYDROCHLORIDE:Atrial fibrillation:0.07), (AZITHROMYCIN MONOHYDRATE:Atrial fibrillation:1.23), (BACITRACIN ZINC:Atrial fibrillation:0.04), (BACITRACIN ZINC\POLYMYXIN B SULFATE:Atrial fibrillation:0.48), (BACITRACIN\NEOMYCIN\POLYMYXIN B:Atrial fibrillation:0.1), (BALSAM PERU OIL\CASTOR OIL\TRYPSIN:Atrial fibrillation:0.3), (BAMIFYLLINE HYDROCHLORIDE:Atrial fibrillation:1.5), (BAZEDOXIFENE ACETATE\ESTROGENS, CONJUGATED:Atrial fibrillation:0.39), (BELIMUMAB:Atrial fibrillation:0.13), (BENFLUOREX:Atrial fibrillation:0.63), (BEPOTASTINE BESILATE:Atrial fibrillation:1.08), (BETAMETHASONE DIPROPIONATE\CALCIPOTRIENE HYDRATE:Atrial fibrillation:0.54), (BETAMETHASONE SODIUM PHOSPHATE\NEOMYCIN SULFATE:Atrial fibrillation:0.75), (BICARBONATE ION\POLYETHYLENE GLYCOLS\POTASSIUM CATION\SODIUM CHLORIDE\SODIUM SULFATE ANHYDROUS:Atrial fibrillation:0.23), (BIFIDOBACTERIUM LONGUM:Atrial fibrillation:0.68), (BISACODYL\GLYCERIN\MINERAL OIL\SODIUM PHOSPHATE, DIBASIC\SODIUM PHOSPHATE, MONOBASIC:Atrial fibrillation:0.1), (BISMUTH SUBSALICYLATE\GLYCERIN\PROPYLENE GLYCOL:Atrial fibrillation:0.11), (BIXALOMER:Atrial fibrillation:1.46), (BLACK COHOSH:Atrial fibrillation:0.49), (BLONANSERIN:Atrial fibrillation:0.55), (BOCEPREVIR:Atrial fibrillation:1.14), (BOTULISM ANTITOXIN:Atrial fibrillation:0.07), (BROMPERIDOL:Atrial fibrillation:0.56), (BUDESONIDE\INDACATEROL MALEATE:Atrial fibrillation:0.01), (BUFORMIN:Atrial fibrillation:1.34), (BUNAZOSIN HYDROCHLORIDE:Atrial fibrillation:0.3), (BUPIVACAINE\EPINEPHRINE:Atrial fibrillation:0.12), (CABOZANTINIB:Atrial fibrillation:1.22), (CAFFEINE\DIPYRONE\ORPHENADRINE:Atrial fibrillation:0.15), (CALCIUM BICARBONATE:Atrial fibrillation:0.86), (CALCIUM CARBONATE\ETIDRONATE DISODIUM:Atrial fibrillation:0.8), (CALCIUM CHLORIDE\DEXTROSE MONOHYDRATE\HYDROCHLORIC ACID\MAGNESIUM CHLORIDE\SODIUM BICARBONATE\SODIUM CHLORIDE\SODIUM HYDROXIDE\SODIUM LACTATE:Atrial fibrillation:0.03), (CALCIUM PANTOTHENATE\CYANOCOBALAMIN\NIACINAMIDE\PYRIDOXINE HYDROCHLORIDE\RIBOFLAVIN\THIAMINE HYDROCHLORIDE:Atrial fibrillation:0.16), (CALCIUM PANTOTHENATE\CYANOCOBALAMIN\NIACINAMIDE\PYRIDOXINE HYDROCHLORIDE\RIBOFLAVIN\THIAMINE MONONITRATE:Atrial fibrillation:1.27), (CALCIUM PHOSPHATE, DIBASIC, ANHYDROUS\FERROUS SUCCINATE\MAGNESIUM HYDROXIDE:Atrial fibrillation:0.28), (CALCIUM\ERGOCALCIFEROL:Atrial fibrillation:0.02), (CAMPHOR\DIPHENHYDRAMINE\ZINC OXIDE:Atrial fibrillation:0.53), (CARBASPIRIN:Atrial fibrillation:0.88), (CARBOXYMETHYLCELLULOSE SODIUM\HYPROMELLOSES:Atrial fibrillation:0.08), (CARPRONIUM CHLORIDE:Atrial fibrillation:0.1), (CEFCAPENE PIVOXIL:Atrial fibrillation:0.09), (CEFOZOPRAN HYDROCHLORIDE:Atrial fibrillation:0.29), (CEFTERAM PIVOXIL:Atrial fibrillation:0.34), (CEPHALOSPORIN C:Atrial fibrillation:0.19), (CEPHRADINE:Atrial fibrillation:0.09), (CERIVASTATIN SODIUM:Atrial fibrillation:1.75), (CHLORAL HYDRATE:Atrial fibrillation:0.12), (CHLOROQUINE PHOSPHATE:Atrial fibrillation:0.01), (CHLORPROMAZINE\PROMETHAZINE:Atrial fibrillation:0.25), (CHOLINE\TRAZODONE HYDROCHLORIDE:Atrial fibrillation:0.32), (CICLOPIROX OLAMINE:Atrial fibrillation:0.76), (CIPROFLOXACIN\DEXAMETHASONE:Atrial fibrillation:0.68), (CITRIC ACID MONOHYDRATE\TRISODIUM CITRATE DIHYDRATE:Atrial fibrillation:0.1), (CLIKSTAR:Atrial fibrillation:1.69), (CLOFAZIMINE:Atrial fibrillation:0.14), (CLOSTRIDIUM TETANI TOXOID ANTIGEN (FORMALDEHYDE INACTIVATED):Atrial fibrillation:0.15), (CLOSTRIDIUM TETANI\CLOSTRIDIUM TETANI TOXOID ANTIGEN (FORMALDEHYDE INACTIVATED)\CORYNEBACTERIUM DIPHTHERIAE\CORYNEBACTERIUM DIPHTHERIAE TOXOID ANTIGEN (FORMALDEHYDE INACTIVATED):Atrial fibrillation:0.36), (COAGULATION FACTOR IX RECOMBINANT HUMAN\ISOPROPYL ALCOHOL:Atrial fibrillation:0.13), (COBICISTAT\ELVITEGRAVIR\EMTRICITABINE\TENOFOVIR ALAFENAMIDE FUMARATE:Atrial fibrillation:0.56), (CODEINE PHOSPHATE\DICLOFENAC SODIUM:Atrial fibrillation:0.13), (CODEINE PHOSPHATE\GUAIFENESIN\PSEUDOEPHEDRINE HYDROCHLORIDE:Atrial fibrillation:0.31), (CODEINE\GUAIFENESIN:Atrial fibrillation:0.17), (COSYNTROPIN:Atrial fibrillation:0.06), (CRANBERRY CONCENTRATE:Atrial fibrillation:0.36), (CREATININE:Atrial fibrillation:0.1), (CUPRIC SULFATE\FERRIC CHLORIDE\MANGANESE CHLORIDE\POTASSIUM IODIDE\ZINC SULFATE:Atrial fibrillation:0.2), (CYANAMIDE:Atrial fibrillation:0.58), (CYANOCOBALAMIN\LIDOCAINE HYDROCHLORIDE\PYRIDOXINE HYDROCHLORIDE\THIAMINE HYDROCHLORIDE:Atrial fibrillation:0.79), (CYANOCOBALAMIN\PYRIDOXAMINE HYDROCHLORIDE\THIAMINE HYDROCHLORIDE:Atrial fibrillation:0.37), (CYANOCOBALAMIN\PYRIDOXINE HYDROCHLORIDE\THIAMINE DISULFIDE:Atrial fibrillation:0.13), (CYSTINE:Atrial fibrillation:0.29), (DASABUVIR SODIUM MONOHYDRATE:Atrial fibrillation:0.08), (DEMECLOCYCLINE:Atrial fibrillation:0.62), (DEOXYRIBONUCLIEC ACID:Atrial fibrillation:0.06), (DESOGESTREL:Atrial fibrillation:0.48), (DEXAMETHASONE\NEOMYCIN:Atrial fibrillation:0.14), (DEXAMETHASONE\NEOMYCIN\POLYMYXIN B SULFATE:Atrial fibrillation:0.14), (DEXAMETHASONE\OXYTETRACYCLINE:Atrial fibrillation:0.12), (DEXTROMETHORPHAN\DEXTROMETHORPHAN HYDROBROMIDE\GUAIFENESIN:Atrial fibrillation:0.02), (DEXTROSE MONOHYDRATE\SODIUM CHLORIDE:Atrial fibrillation:0.06), (DIACEREIN:Atrial fibrillation:0.44), (DIFLUCORTOLONE VALERATE\LIDOCAINE:Atrial fibrillation:0.43), (DILAZEP DIHYDROCHLORIDE:Atrial fibrillation:0.08), (DIMETHICONE 410:Atrial fibrillation:0.56), (DIMETHYL SULFOXIDE:Atrial fibrillation:0.27), (DIPHENHYDRAMINE HYDROCHLORIDE\DOXYLAMINE SUCCINATE:Atrial fibrillation:0.03), (DIPYRONE\PROMETHAZINE HYDROCHLORIDE:Atrial fibrillation:1.05), (DOLUTEGRAVIR:Atrial fibrillation:0.36), (DROSPIRENONE\ETHINYL ESTRADIOL:Atrial fibrillation:0.7), (DROTRECOGIN ALFA (ACTIVATED):Atrial fibrillation:0.48), (DUPILUMAB:Atrial fibrillation:0.05), (DYDROGESTERONE:Atrial fibrillation:0.59), (DYPHYLLINE:Atrial fibrillation:0.16), (ECABET SODIUM:Atrial fibrillation:1.73), (ECALLANTIDE:Atrial fibrillation:0.12), (ELECTROLYTES NOS\POLYETHYLENE GLYCOL 3350:Atrial fibrillation:0.01), (EMTRICITABINE\RILPIVIRINE HYDROCHLORIDE\TENOFOVIR DISOPROXIL FUMARATE:Atrial fibrillation:0.31), (EMTRICITABINE\RILPIVIRINE\TENOFOVIR DISOPROXIL FUMARATE:Atrial fibrillation:0.13), (EPHEDRINE HYDROCHLORIDE\GUAIFENESIN:Atrial fibrillation:0.07), (ERGOLOID MESYLATES:Atrial fibrillation:0.77), (ERIBULIN:Atrial fibrillation:0.77), (ESCHERICHIA COLI:Atrial fibrillation:0.15), (ESTRAMUSTINE:Atrial fibrillation:0.03), (ESTROGENS\METHYLTESTOSTERONE:Atrial fibrillation:0.08), (ETHINYL ESTRADIOL\GESTODENE:Atrial fibrillation:0.17), (ETHINYL ESTRADIOL\NORGESTIMATE:Atrial fibrillation:0.23), (ETHINYL ESTRADIOL\NORGESTREL:Atrial fibrillation:0.63), (ETRETINATE:Atrial fibrillation:2.82), (EUCALYPTOL\MENTHOL\METHYL SALICYLATE\THYMOL:Atrial fibrillation:0.32), (FENOPROFEN:Atrial fibrillation:0.08), (FERROUS BISGLYCINATE:Atrial fibrillation:1.4), (FERUMOXYTOL:Atrial fibrillation:0.34), (FLOMOXEF:Atrial fibrillation:0.52), (FLOMOXEF SODIUM:Atrial fibrillation:0), (FLUNARIZINE HYDROCHLORIDE:Atrial fibrillation:1.39), (FLUOXETINE HYDROCHLORIDE\OLANZAPINE:Atrial fibrillation:0.15), (FLUPENTIXOL DIHYDROCHLORIDE\MELITRACEN HYDROCHLORIDE:Atrial fibrillation:0.1), (FLUPHENAZINE:Atrial fibrillation:0.57), (FLURANDRENOLIDE:Atrial fibrillation:0.18), (FLUTOPRAZEPAM:Atrial fibrillation:0.17), (FOSBRETABULIN TROMETHAMINE:Atrial fibrillation:0.09), (FOSCARNET SODIUM:Atrial fibrillation:0.1), (FOSFLUCONAZOLE:Atrial fibrillation:0.32), (FOSFOMYCIN CALCIUM:Atrial fibrillation:0.84), (FOSINOPRIL SODIUM\HYDROCHLOROTHIAZIDE:Atrial fibrillation:1.45), (FURSULTIAMINE:Atrial fibrillation:2.26), (FUSIDATE SODIUM:Atrial fibrillation:0.64), (GADOBUTROL:Atrial fibrillation:0.59), (GADOPENTETATE DIMEGLUMINE:Atrial fibrillation:0.07), (GENTIAN VIOLET:Atrial fibrillation:0.46), (GLIMEPIRIDE\PIOGLITAZONE HYDROCHLORIDE:Atrial fibrillation:0.28), (GLIMEPIRIDE\ROSIGLITAZONE MALEATE:Atrial fibrillation:2), (GLUCONATE:Atrial fibrillation:0.39), (GLYCERIN\HYPROMELLOSE 2910 (15 MPA.S)\POLYETHYLENE GLYCOL 400\PROPYLENE GLYCOL:Atrial fibrillation:0.24), (GLYCOPYRRONIUM\FORMOTEROL FUMARATE:Atrial fibrillation:0.65), (GRISEOFULVIN:Atrial fibrillation:0.01), (GUAIAZULENE:Atrial fibrillation:0.11), (GUAIFENESIN\PHENYLEPHRINE HYDROCHLORIDE:Atrial fibrillation:0.05), (GUANABENZ ACETATE:Atrial fibrillation:1.89), (HALCINONIDE:Atrial fibrillation:0.46), (HEPARIN SODIUM\SODIUM CHLORIDE:Atrial fibrillation:0.08), (HERBALS\KARAYA GUM:Atrial fibrillation:1.05), (HERBS\ROOTS:Atrial fibrillation:0.1), (HORSE CHESTNUT:Atrial fibrillation:0.74), (HYDROCHLORIC ACID:Atrial fibrillation:0.18), (HYDROCORTISONE HEMISUCCINATE:Atrial fibrillation:0.43), (HYDROCORTISONE PROBUTATE:Atrial fibrillation:0.15), (HYDROCORTISONE VALERATE:Atrial fibrillation:0.19), (HYDROXYPROPYL CELLULOSE:Atrial fibrillation:0.27), (IBRITUMOMAB TIUXETAN:Atrial fibrillation:1.1), (IGURATIMOD:Atrial fibrillation:1.66), (IMMUNE GLOBULIN NOS:Atrial fibrillation:0.65), (INSULIN ASPART\INSULIN DEGLUDEC:Atrial fibrillation:2.26), (INSULIN PORKINSULIN PURIFIED PORK:Atrial fibrillation:1.9), (INTERFERON ALFA-2A:Atrial fibrillation:2.54), (INTERFERON ALFA-2A OR INTERFERON ALFA-2B:Atrial fibrillation:0.68), (INTERFERON ALFA-N1:Atrial fibrillation:0.42), (INTERFERON GAMMA-1B:Atrial fibrillation:0.92), (IODINE\POTASSIUM IODIDE:Atrial fibrillation:0.04), (IPRAGLIFLOZIN:Atrial fibrillation:1.15), (ISOFLAVONES SOY:Atrial fibrillation:0.07), (ISTRADEFYLLINE:Atrial fibrillation:0.06), (ITRACONAZOLE\SODIUM CHLORIDE:Atrial fibrillation:0.94), (IVACAFTOR:Atrial fibrillation:0.4), (IXABEPILONE:Atrial fibrillation:0.29), (KAOLIN:Atrial fibrillation:0.14), (LAPATINIB:Atrial fibrillation:0.27), (LATANOPROST\TIMOLOL:Atrial fibrillation:1.28), (LECITHIN\SODIUM BICARBONATE\SODIUM PHOSPHATE, MONOBASIC:Atrial fibrillation:0.34), (LEVOCETIRIZINE HYDROCHLORIDE:Atrial fibrillation:0.19), (LOBUCAVIR:Atrial fibrillation:0.6), (LOMUSTINE:Atrial fibrillation:0.3), (LUMACAFTOR:Atrial fibrillation:0.07), (LUTETIUM LU-177:Atrial fibrillation:1.32), (LYNESTRENOL:Atrial fibrillation:0.01), (MAGNESIUM SULFATE\POTASSIUM SULFATE\SODIUM SULFATE:Atrial fibrillation:0.1), (MECLOZINE DIHYDROCHLORIDE:Atrial fibrillation:0.25), (MEPENZOLATE BROMIDE:Atrial fibrillation:1.22), (MEPREDNISONE:Atrial fibrillation:0.04), (METFORMIN HYDROCHLORIDE\ROSIGLITAZONE MALEATE:Atrial fibrillation:2.19), (METFORMIN\VILDAGLIPTIN:Atrial fibrillation:0.4), (METHENAMINE MANDELATE:Atrial fibrillation:0.78), (METHENOLONE ACETATE:Atrial fibrillation:0.33), (METHYLENE BLUE:Atrial fibrillation:0.29), (MIGLUSTAT:Atrial fibrillation:0.24), (MILNACIPRAN:Atrial fibrillation:0.72), (MINERALS\PROBIOTICS NOS\VITAMINS:Atrial fibrillation:0.15), (MITOXANTRONE HYDROCHLORIDE:Atrial fibrillation:0.84), (MORPHINE\MORPHINE HYDROCHLORIDE\MORPHINE SULFATE\MORPHINE TARTRATE:Atrial fibrillation:0.12), (NABILONE:Atrial fibrillation:0.42), (NABIXIMOLS:Atrial fibrillation:0.43), (NADIFLOXACIN:Atrial fibrillation:0.36), (NAFAMOSTAT:Atrial fibrillation:0.08), (NALDEMEDINE:Atrial fibrillation:0.56), (NALFURAFINE HYDROCHLORIDE:Atrial fibrillation:0.62), (NAPROXEN SODIUM\SUMATRIPTAN SUCCINATE:Atrial fibrillation:0.21), (NARATRIPTAN HYDROCHLORIDE:Atrial fibrillation:1.02), (NEFAZODONE:Atrial fibrillation:0.7), (NEFAZODONE HYDROCHLORIDE:Atrial fibrillation:0.9), (NIAOULI OIL\QUININE BENZOATE\THIAMINE HYDROCHLORIDE:Atrial fibrillation:0.63), (NIFURTOINOL:Atrial fibrillation:0.11), (NIPRADILOL:Atrial fibrillation:0.01), (NITROGEN:Atrial fibrillation:0.1), (OBETICHOLIC ACID:Atrial fibrillation:0.53), (OCRELIZUMAB:Atrial fibrillation:0.15), (OK-432:Atrial fibrillation:0.26), (ORGOTEIN:Atrial fibrillation:0.1), (OSTREA EDULIS SHELL:Atrial fibrillation:0.43), (OXETHAZAINE:Atrial fibrillation:0.37), (OXETORONE:Atrial fibrillation:0.57), (PANTHENOL:Atrial fibrillation:0.23), (PARATHYROID HORMONE:Atrial fibrillation:0.79), (PEGVISOMANT:Atrial fibrillation:0.66), (PENICILLAMINE:Atrial fibrillation:0.74), (PENICILLIN V BENZATHINE:Atrial fibrillation:1.1), (PENTOSTATIN:Atrial fibrillation:1.62), (PEROSPIRONE:Atrial fibrillation:0.11), (PHENOXYBENZAMINE:Atrial fibrillation:0.08), (PHENTERMINE HYDROCHLORIDE:Atrial fibrillation:0.06), (PHENYLEPHRINE HYDROCHLORIDE\WITCH HAZEL:Atrial fibrillation:0.58), (PIRENZEPINE:Atrial fibrillation:0.63), (PLASMA PROTEIN FRACTION (HUMAN):Atrial fibrillation:0.12), (PLERIXAFOR:Atrial fibrillation:0.34), (PODOFILOX:Atrial fibrillation:0.08), (POLIDOCANOL:Atrial fibrillation:0.12), (POLYMYXIN B SULFATE:Atrial fibrillation:0.05), (POLYMYXIN B SULFATE\TRIMETHOPRIM SULFATE:Atrial fibrillation:0.47), (PORFIMER SODIUM:Atrial fibrillation:0.65), (POTASSIUM PHOSPHATE, DIBASIC\POTASSIUM PHOSPHATE, MONOBASIC\SODIUM PHOSPHATE, DIBASIC\SODIUM PHOSPHATE, MONOBASIC:Atrial fibrillation:0.01), (POTASSIUM SORBATE:Atrial fibrillation:0.05), (PRAMOXINE HYDROCHLORIDE:Atrial fibrillation:0.68), (PRANOPROFEN:Atrial fibrillation:0.09), (PREDNISOLONE ACETATE\SULFACETAMIDE SODIUM:Atrial fibrillation:0.49), (PREDNISOLONE SODIUM METAZOATE:Atrial fibrillation:0.45), (PREDNISOLONE SODIUM PHOSPHATE:Atrial fibrillation:0.4), (PREDNISOLONE VALERATE ACETATE:Atrial fibrillation:0.84), (PREGNENOLONE:Atrial fibrillation:0.2), (PROCYCLIDINE:Atrial fibrillation:0.57), (PROPANTHELINE BROMIDE:Atrial fibrillation:0.09), (PROTRIPTYLINE HYDROCHLORIDE:Atrial fibrillation:1.35), (PRUCALOPRIDE SUCCINATE:Atrial fibrillation:1.45), (PSORALEN:Atrial fibrillation:0.29), (PYRIDOXAL PHOSPHATE:Atrial fibrillation:1.22), (PYRIMETHAMINE:Atrial fibrillation:0.95), (QUINAGOLIDE HYDROCHLORIDE:Atrial fibrillation:0.42), (QUININE BISULFATE:Atrial fibrillation:0.4), (RANITIDINE BISMUTH CITRATE:Atrial fibrillation:0.41), (RASAGILINE:Atrial fibrillation:0.5), (RETEPLASE:Atrial fibrillation:0.72), (RHUBARB:Atrial fibrillation:0.84), (RIBOCICLIB:Atrial fibrillation:0.26), (RILOTUMUMAB:Atrial fibrillation:0.07), (RILPIVIRINE:Atrial fibrillation:0.26), (RILUZOLE:Atrial fibrillation:0.14), (RIMABOTULINUMTOXINB:Atrial fibrillation:0.03), (RIZATRIPTAN:Atrial fibrillation:1.29), (ROPIVACAINE HYDROCHLORIDE:Atrial fibrillation:1.17), (ROSIGLITAZONE:Atrial fibrillation:1.53), (ROXADUSTAT:Atrial fibrillation:0.88), (SENNOSIDE A\SENNOSIDE B\SENNOSIDES:Atrial fibrillation:0.24), (SENNOSIDE B:Atrial fibrillation:1.26), (SIBUTRAMINE HYDROCHLORIDE:Atrial fibrillation:1.2), (SIVELESTAT SODIUM:Atrial fibrillation:0.92), (SODIUM GUALENATE MONOHYDRATE:Atrial fibrillation:0.67), (SODIUM PHOSPHATE, DIBASIC:Atrial fibrillation:0.46), (SOFOSBUVIR\VELPATASVIR:Atrial fibrillation:1.2), (SUCCINYLCHOLINE CHLORIDE:Atrial fibrillation:1.42), (SULFADIAZINE:Atrial fibrillation:0.12), (SULFAMERAZINE\TRIMETHOPRIM:Atrial fibrillation:0.09), (SULTAMICILLIN:Atrial fibrillation:0.3), (SUPLATAST TOSILATE:Atrial fibrillation:2.17), (SUPROFEN:Atrial fibrillation:0.79), (TALIGLUCERASE ALFA:Atrial fibrillation:0.05), (TALTIRELIN:Atrial fibrillation:0.07), (TANDOSPIRONE:Atrial fibrillation:0.47), (TECELEUKIN:Atrial fibrillation:0.02), (TEDUGLUTIDE:Atrial fibrillation:0.92), (TEGASEROD:Atrial fibrillation:0.04), (TEGASEROD MALEATE:Atrial fibrillation:0.37), (TENECTEPLASE:Atrial fibrillation:1.73), (THIETHYLPERAZINE MALATE:Atrial fibrillation:0.06), (THIOPENTAL SODIUM:Atrial fibrillation:0.13), (THIOTHIXENE HYDROCHLORIDE:Atrial fibrillation:0.16), (THYMOL:Atrial fibrillation:0.1), (TIAPROFENIC ACID:Atrial fibrillation:0.7), (TIARAMIDE:Atrial fibrillation:0.48), (TIBOLONE:Atrial fibrillation:1.01), (TIOPRONIN:Atrial fibrillation:0.03), (TOBRAMYCIN SULFATE:Atrial fibrillation:0.19), (TOLCAPONE:Atrial fibrillation:0.2), (TOREMIFENE CITRATE:Atrial fibrillation:0.29), (TOSUFLOXACIN TOSYLATE:Atrial fibrillation:0.53), (TRANILAST:Atrial fibrillation:0.29), (TREPROSTINIL SODIUM:Atrial fibrillation:0.46), (TRIHEXYPHENIDYL:Atrial fibrillation:1.52), (TROGLITAZONE:Atrial fibrillation:0.39), (TROMETHAMINE:Atrial fibrillation:0.02), (URACIL:Atrial fibrillation:0.19), (VALDECOXIB:Atrial fibrillation:1.64), (VITAMIN A PALMITATE:Atrial fibrillation:0.13), (VORAPAXAR SULFATE:Atrial fibrillation:1.38), (XYLOMETAZOLINE:Atrial fibrillation:0.22), (ZALTOPROFEN:Atrial fibrillation:1.06), (ZIDOVUDINE:Atrial fibrillation:0.2), (ZINC CITRATE:Atrial fibrillation:0.55), (ZIPRASIDONE HYDROCHLORIDE:Atrial fibrillation:0.13), (ZIV-AFLIBERCEPT:Atrial fibrillation:1.06), (ZOTEPINE:Atrial fibrillation:0.25), (ACARBOSE:Atrial flutter:0.35), (ALBIGLUTIDE:Atrial flutter:0.13), (ALFACALCIDOL:Atrial flutter:0.11), (AMLODIPINE\VALSARTAN:Atrial flutter:0.01),(ARMODAFINIL:Atrial flutter:0.15), (ATROPINE SULFATE\DIPHENOXYLATE HYDROCHLORIDE:Atrial flutter:0.26), (BENZONATATE:Atrial flutter:0.19), (BISACODYL:Atrial flutter:0.44), (BUDESONIDE\FORMOTEROL FUMARATE:Atrial flutter:0.25), (CALCIUM ACETATE:Atrial flutter:0.91), (CANAGLIFLOZIN:Atrial flutter:0.18), (CARFILZOMIB:Atrial flutter:0.39), (CARVEDILOL PHOSPHATE:Atrial flutter:0.91), (CEFOTAXIME SODIUM:Atrial flutter:0.08), (CERTOLIZUMAB PEGOL:Atrial flutter:0.74), (CHLORTHALIDONE:Atrial flutter:0.15), (CHOLESTYRAMINE:Atrial flutter:0.61), (CINACALCET HYDROCHLORIDE:Atnal flutter:0.37), (CLINDAMYCIN\CLINDAMYCIN PHOSPHATE:Atrial flutter:1.01), (COLESEVELAM HYDROCHLORIDE:Atrial flutter:0.14), (CORTISONE\HYDROCORTISONE:Atrial flutter:0.4), (DASATINIB:Atrial flutter:1.57), (DICYCLOMINE HYDROCHLORIDE:Atrial flutter:0.47), (DOCUSATE CALCIUM:Atrial flutter:0.03), (DOMPERIDONE:Atrial flutter:0.05), (ECONAZOLE:Atrial flutter:0.08), (ELETRIPTAN HYDROBROMIDE:Atrial flutter:0.13), (EPINEPHRINE:Atrial flutter:0.57), (ERYTHROPOIETIN:Atrial flutter:1.11), (EVEROLIMUS:Atrial flutter:0.62), (EVOLOCUMAB:Atrial flutter:1.3), (FLUTICASONE FUROATE\SALMETEROL XINAFOATE:Atrial flutter:0.43), (FORMOTEROL FUMARATE:Atrial flutter:0.18), (GLYCOPYRRONIUM\INDACATEROL:Atrial flutter:0.47), (HYDROXYCHLOROQUINE SULFATE:Atrial flutter:1), (HYDROXYZINE PAMOATE:Atrial flutter:0.54), (HYOSCYAMINE:Atrial flutter:0.18), (ILOPROST:Atrial flutter:0.96), (INDAPAMIDE\PERINDOPRIL:Atrial flutter:0.98), (INSULIN HUMAN:Atrial flutter:1), (IPRATROPIUM:Atrial flutter:1.16), (LEDIPASVIR\SOFOSBUVIR:Atrial flutter:0.94), (LINAGLIPTIN:Atrial flutter:1.02), (LITHIUM CARBONATE:Atrial flutter:0.31), (METAMIZOLE:Atrial flutter:0.15), (METAXALONE:Atrial flutter:0.59), (METHADONE HYDROCHLORIDE:Atrial flutter:0.45), (METRONIDAZOLE:Atrial flutter:1.08), (NILOTINIB:Atrial flutter:1.31), (NINTEDANIB:Atrial flutter:0.11), (NITROFURANTOIN:Atrial flutter:0.49), (NORTRIPTYLINE:Atrial flutter:0.19), (PANTOPRAZOLE MAGNESIUM:Atnial flutter:0.49), (PAZOPANIB HYDROCHLORIDE:Atrial flutter:0.43), (PEMETREXED DISODIUM:Atrial flutter:0.34), (PENICILLIN:Atrial flutter:0.17), (PHENOBARBITAL:Atrial flutter:0.12), (PLANTAGO SEED:Atrial flutter:0.03), (PREDNISOLONE\PREDNISOLONE ACETATE\PREDNISOLONE SODIUM SUCCINATE:Atrial flutter:0.05), (PRIMIDONE:Atrial flutter:0.7), (PYRIDOXINE HYDROCHLORIDE:Atrial flutter:0.24), (ROFLUMILAST:Atrial flutter:0.23), (ROSIGLITAZONE MALEATE:Atrial flutter:0.16), (SELEXIPAG:Atrial flutter:0.38), (SODIUM OXYBATE:Atrial flutter:0.41), (SUMATRIPTAN\SUMATRIPTAN SUCCINATE:Atrial flutter:0.02), (TETRACYCLINE:Atrial flutter:0.35), (THALIDOMIDE:Atrial flutter:0.48), (TOPIRAMATE:Atrial flutter:1.09), (VALGANCICLOVIR HYDROCHLORIDE:Atrial flutter:0.13), (VARENICLINE TARTRATE:Atrial flutter:0.21), (VINCRISTINE SULFATE:Atrial flutter:0.27), (EVOLOCUMAB:Atrial tachycardia:0.09), (FERROUS SULFATE\FERROUS SULFATE, DRIED:Atrial tachycardia:0.06), (ILOPROST:Atrial tachycardia:0.17), (LOVASTATIN:Atrial tachycardia:0.27), (METHYLPREDNISOLONE SODIUM SUCCINATE:Atrial tachycardia:0.03), (NAPROXEN:Atrial tachycardia:0.24), (PANTOPRAZOLE:Atrial tachycardia:0.17), (TAMSULOSIN:Atrial tachycardia:0.02), (TOFACITINIB CITRATE:Atrial tachycardia:0.28), (ANASTROZOLE:Atrophic vulvovaginitis:0.12),(ARMODAFINIL:Atrophic vulvovaginitis:0.24), (CALCIUM CARBONATE\VITAMIN D:Atrophic vulvovaginitis:0.27), (CERTOLIZUMAB PEGOL:Atrophic vulvovaginitis:0.62), (DALFAMPRIDINE:Atrophic vulvovaginitis:0.74), (ELETRIPTAN HYDROBROMIDE:Atrophic vulvovaginitis:0.08), (EVOLOCUMAB:Atrophic vulvovaginitis:0.36), (FERROUS SULFATE:Atrophic vulvovaginitis:0.25), (FERROUS SULFATE\FERROUS SULFATE, DRIED:Atrophic vulvovaginitis:0.18), (GLIMEPIRIDE:Atrophic vulvovaginitis:0.25), (IBRUTINIB:Atrophic vulvovaginitis:0.56), (METAXALONE:Atrophic vulvovaginitis:0.24), (METHYLPREDNISOLONE SODIUM SUCCINATE:Atrophic vulvovaginitis:0.4), (OXCARBAZEPINE:Atrophic vulvovaginitis:0.39), (OXYGEN:Atrophic vulvovaginitis:0.23), (PRIMIDONE:Atrophic vulvovaginitis:0.1), (SACUBITRIL\VALSARTAN:Atrophic vulvovaginitis:0.01), (SODIUM CHLORIDE:Atrophic vulvovaginitis:0.05), (SODIUM OXYBATE:Atrophic vulvovaginitis:0.07), (TAMSULOSIN:Atrophic vulvovaginitis:0.02), (VARENICLINE TARTRATE:Atrophic vulvovaginitis:0.18), (.ALPHA.1-PROTEINASE INHIBITOR HUMAN:Attention deficit/hyperactivity disorder:1.49), (ACARBOSE:Attention deficit/hyperactivity disorder:0.06), (ACETAMINOPHEN\DICHLORALPHENAZONE\ISOMETHEPTENE:Attentio n deficit/hyperactivity disorder:1.04), (ACETAMINOPHEN\TRAMADOL HYDROCHLORIDE:Attention deficit/hyperactivity disorder:0.14), (ALENDRONIC ACID:Attention deficit/hyperactivity disorder:0.37), (AMILORIDE HYDROCHLORIDE\HYDROCHLOROTHIAZIDE:Attention deficit/hyperactivity disorder:0.31), (AMINO ACIDS\DEXTROSE\ELECTROLYTES NOS:Attention deficit/hyperactivity disorder:0.24), (AMIODARONE HYDROCHLORIDE:Attention deficit/hyperactivity disorder:0.55), (AMLODIPINE BESYLATE\HYDROCHLOROTHIAZIDE\OLMESARTAN MEDOXOMIL:Attention deficit/hyperactivity disorder:0.65), (AMLODIPINE\VALSARTAN:Attention deficit/hyperactivity disorder:0.48), (AMMONIUM CHLORIDE\DEXTROMETHORPHAN\SODIUM CITRATE:Attention deficit/hyperactivity disorder:0.13), (AMMONIUM LACTATE:Attention deficit/hyperactivity disorder:0.77), (ANHYDROUS CITRIC ACID\ASPIRIN\SODIUM BICARBONATE:Attention deficit/hyperactivity disorder:0.05), (BENAZEPRIL\BENAZEPRIL HYDROCHLORIDE:Attention deficit/hyperactivity disorder:0.66), (BETAMETHASONE DIPROPIONATE\CALCIPOTRIENE\CALCIPOTRIENE HYDRATE:Attention deficit/hyperactivity disorder:1.01), (BETAMETHASONE DIPROPIONATE\CLOTRIMAZOLE:Attention deficit/hyperactivity disorder:0.01), (BETAMETHASONE\CALCIPOTRIENE:Attention deficit/hyperactivity disorder:0.11), (BICALUTAMIDE:Attention deficit/hyperactivity disorder:0.91), (BISOPROLOL FUMARATE:Attention deficit/hyperactivity disorder:1.11), (BRINZOLAMIDE:Attention deficit/hyperactivity disorder:0.42), (BUTABARBITAL:Attention deficit/hyperactivity disorder:0.19), (BUTORPHANOL:Attention deficit/hyperactivity disorder:0.12), (CALCIPOTRIENE:Attention deficit/hyperactivity disorder:0.36), (CALCITONIN:Attention deficit/hyperactivity disorder:0.01), (CALCIUM CITRATE\VITAMIN D:Attention deficit/hyperactivity disorder:0.21), (CALCIUM\MAGNESIUM:Attention deficit/hyperactivity disorder:0.54), (CALCIUM\MAGNESIUM\ZINC:Attention deficit/hyperactivity disorder:0.89), (CANDESARTAN:Attention deficit/hyperactivity disorder:1.38), (CANDESARTAN CILEXETIL\HYDROCHLOROTHIAZIDE:Attention deficit/hyperactivity disorder:1.01), (CAPSAICIN:Attention deficit/hyperactivity disorder:0.59), (CARVEDILOL PHOSPHATE:Attention deficit/hyperactivity disorder:1.48), (CEFTRIAXONE SODIUM:Attention deficit/hyperactivity disorder:0.98), (CETIRIZINE HYDROCHLORIDE\PSEUDOEPHEDRINE HYDROCHLORIDE:Attention deficit/hyperactivity disorder:1.29), (CILOSTAZOL:Attention deficit/hyperactivity disorder:0.05), (CODEINE PHOSPHATE:Attention deficit/hyperactivity disorder:0.35), (COLESTIPOL:Attention deficit/hyperactivity disorder:0.32), (CORTICOTROPIN:Attention deficit/hyperactivity disorder:1), (CREATINE MONOHYDRATE:Attention deficit/hyperactivity disorder:0.07), (DACLIZUMAB:Attention deficit/hyperactivity disorder:1.42), (DALTEPARIN:Attention deficit/hyperactivity disorder:0), (DAPAGLIFLOZIN PROPANEDIOL:Attention deficit/hyperactivity disorder:0.65), (DARBEPOETIN ALFA:Attention deficit/hyperactivity disorder:0.47), (DARUNAVIR ETHANOLATE:Attention deficit/hyperactivity disorder:0.22), (DEFLAZACORT:Attention deficit/hyperactivity disorder:0.82), (DIENOGEST:Attention deficit/hyperactivity disorder:0.28), (DIFLUNISAL:Attention deficit/hyperactivity disorder:0.33), (DIPYRONE:Attention deficit/hyperactivity disorder:0.42), (DIPYRONE\PROMETHAZINE HYDROCHLORIDE:Attention deficit/hyperactivity disorder:0.03), (DORZOLAMIDE:Attention deficit/hyperactivity disorder:0.1), (DUTASTERIDE\TAMSULOSIN HYDROCHLORIDE:Attention deficit/hyperactivity disorder:0.46), (ELTROMBOPAG OLAMINE:Attention deficit/hyperactivity disorder:0.29), (EMPAGLIFLOZIN\LINAGLIPTIN:Attention deficit/hyperactivity disorder:0.24), (EMTRICITABINE\RILPIVIRINE HYDROCHLORIDE\TENOFOVIR DISOPROXIL FUMARATE:Attention deficit/hyperactivity disorder:0.03), (ENOXAPARIN:Attention deficit/hyperactivity disorder:1.42), (EPHEDRINE HYDROCHLORIDE\GUAIFENESIN:Attention deficit/hyperactivity disorder:0.26), (EPLERENONE:Attention deficit/hyperactivity disorder:0.14), (ESTRADIOL BENZOATE:Attention deficit/hyperactivity disorder:0.25), (ESTRADIOL\LEVONORGESTREL:Attention deficit/hyperactivity disorder:0.57), (EVOLOCUMAB:Attention deficit/hyperactivity disorder:2.04), (FERROUS BISGLYCINATE:Attention deficit/hyperactivity disorder:0.01), (FLUTICASONE FUROATE\SALMETEROL XINAFOATE:Attention deficit/hyperactivity disorder:1.02), (FORMOTEROL:Attention deficit/hyperactivity disorder:0.17), (FORMOTEROL FUMARATE:Attention deficit/hyperactivity disorder:0.86), (FORMOTEROL FUMARATE DIHYDRATE:Attention deficit/hyperactivity disorder:0.02), (FOSPHENYTOIN\FOSPHENYTOIN SODIUM:Attention deficit/hyperactivity disorder:0.06), (FUSIDIC ACID:Attention deficit/hyperactivity disorder:0.37), (GLICLAZIDE:Attention deficit/hyperactivity disorder:0.58), (GLYCOPYRRONIUM:Attention deficit/hyperactivity disorder:0.35), (GLYCOPYRRONIUM\INDACATEROL:Attention deficit/hyperactivity disorder:0.51), (HEMP:Attention deficit/hyperactivity disorder:0.12), (HYDROXYCHLOROQUINE DIPHOSPHATE:Attention deficit/hyperactivity disorder:0.57), (HYPERICUM PERFORATUM:Attention deficit/hyperactivity disorder:0.29), (ICOSAPENT ETHYL:Attention deficit/hyperactivity disorder:0.08), (INDACATEROL:Attention deficit/hyperactivity disorder:0.13), (INDAPAMIDE\PERINDOPRIL:Attention deficit/hyperactivity disorder:0.06), (INFLIXIMAB-DYYB:Attention deficit/hyperactivity disorder:0.21), (INSULIN BEEF:Attention deficit/hyperactivity disorder:0.1), (INTERFERON GAMMA-1B:Attention deficit/hyperactivity disorder:0.23), (ISOSORBIDE:Attention deficit/hyperactivity disorder:1), (KETOPROFEN:Attention deficit/hyperactivity disorder:0.22), (LABETALOL HYDROCHLORIDE:Attention deficit/hyperactivity disorder:0.95), (LANREOTIDE ACETATE:Attention deficit/hyperactivity disorder:0.45), (LARONIDASE:Attention deficit/hyperactivity disorder:0.47), (LEVALBUTEROL TARTRATE:Attention deficit/hyperactivity disorder:0.04), (LIDOCAINE HYDROCHLORIDE:Attention deficit/hyperactivity disorder:0.93), (LURASIDONE:Attention deficit/hyperactivity disorder:0.09), (LYSERGIDE:Attention deficit/hyperactivity disorder:0.51), (MESTRANOL\NORETHINDRONE:Attention deficit/hyperactivity disorder:0.22), (METHENAMINE HIPPURATE:Attention deficit/hyperactivity disorder:1.09), (METHSUXIMIDE:Attention deficit/hyperactivity disorder:0.09), (METHYLPREDNISOLONE ACETATE:Attention deficit/hyperactivity disorder:1.55), (MIDODRINE HYDROCHLORIDE:Attention deficit/hyperactivity disorder:0.79), (MIGLUSTAT:Attention deficit/hyperactivity disorder:0.29), (MORPHINE\MORPHINE HYDROCHLORIDE\MORPHINE SULFATE\MORPHINE TARTRATE:Attention deficit/hyperactivity disorder:0.17), (NALBUPHINE HYDROCHLORIDE:Attention deficit/hyperactivity disorder:0.15), (NALOXONE:Attention deficit/hyperactivity disorder:0.19), (NALOXONE HYDROCHLORIDE\OXYCODONE HYDROCHLORIDE:Attention deficit/hyperactivity disorder:0.68), (NEBIVOLOL:Attention deficit/hyperactivity disorder:0.03), (NIRAPARIB:Attention deficit/hyperactivity disorder:0.04), (OLODATEROL HYDROCHLORIDE\TIOTROPIUM BROMIDE MONOHYDRATE:Attention deficit/hyperactivity disorder:0.37), (OXAPROZIN:Attention deficit/hyperactivity disorder:0.06), (OXYBUTYNIN\OXYBUTYNIN CHLORIDE:Attention deficit/hyperactivity disorder:0.05), (PAMIDRONATE DISODIUM:Attention deficit/hyperactivity disorder:0.39), (PANCRELIPASE:Attention deficit/hyperactivity disorder:0.51), (PANTOPRAZOLE MAGNESIUM:Attention deficit/hyperactivity disorder:1.84), (PASIREOTIDE:Attention deficit/hyperactivity disorder:0.25), (PATIROMER:Attention deficit/hyperactivity disorder:0.03), (PAZOPANIB HYDROCHLORIDE:Attention deficit/hyperactivity disorder:1.01), (PEGFILGRASTIM:Attention deficit/hyperactivity disorder:0.43), (PEGINTERFERON BETA-1A:Attention deficit/hyperactivity disorder:1.44), (PEROSPIRONE:Attention deficit/hyperactivity disorder:0.06), (PHENYTOIN SODIUM:Attention deficit/hyperactivity disorder:0.99), (PIMAVANSERIN TARTRATE:Attention deficit/hyperactivity disorder:0.02), (PIOGLITAZONE:Attention deficit/hyperactivity disorder:0.25), (PIRBUTEROL ACETATE:Attention deficit/hyperactivity disorder:0.28), (PIROXICAM:Attention deficit/hyperactivity disorder:0.52), (PONATINIB:Attention deficit/hyperactivity disorder:0.62), (PREDNISOLONE SODIUM PHOSPHATE:Attention deficit/hyperactivity disorder:0.84), (PROCYCLIDINE:Attention deficit/hyperactivity disorder:0.41), (PROTRIPTYLINE HYDROCHLORIDE:Attention deficit/hyperactivity disorder:0.97), (QUINAPRIL:Attention deficit/hyperactivity disorder:0.16), (RALOXIFENE HYDROCHLORIDE:Attention deficit/hyperactivity disorder:1.28), (RASAGILINE MESYLATE:Attention deficit/hyperactivity disorder:0.09), (REBOXETINE:Attention deficit/hyperactivity disorder:0.01), (RISEDRONATE SODIUM:Attention deficit/hyperactivity disorder:0.78), (RIVASTIGMINE:Attention deficit/hyperactivity disorder:1.24), (ROFECOXIB:Attention deficit/hyperactivity disorder:0.01), (ROFLUMILAST:Attention deficit/hyperactivity disorder:1.2), (ROTIGOTINE:Attention deficit/hyperactivity disorder:0.32), (RUFINAMIDE:Attention deficit/hyperactivity disorder:0.65), (SACCHAROMYCES CEREVISIAE:Attention deficit/hyperactivity disorder:0.3), (SACUBITRIL\VALSARTAN:Attention deficit/hyperactivity disorder:0.21), (SERTINDOLE:Attention deficit/hyperactivity disorder:0.27), (SIBUTRAMINE HYDROCHLORIDE:Attention deficit/hyperactivity disorder:0.07), (SITAGLIPTIN:Attention deficit/hyperactivity disorder:0.44), (SULFACETAMIDE SODIUM\SULFUR:Attention deficit/hyperactivity disorder:0.02), (TAZAROTENE:Attention deficit/hyperactivity disorder:0.04), (TEDUGLUTIDE\WATER:Attention deficit/hyperactivity disorder:0.12), (TERCONAZOLE:Attention deficit/hyperactivity disorder:0.13), (THEOPHYLLINE:Attention deficit/hyperactivity disorder:0.12), (THIAMINE:Attention deficit/hyperactivity disorder:0.18), (TIBOLONE:Attention deficit/hyperactivity disorder:0.02), (TIOTROPIUM:Attention deficit/hyperactivity disorder:0.36), (TROSPIUM CHLORIDE:Attention deficit/hyperactivity disorder:0.59), (UMECLIDINIUM BROMIDE:Attention deficit/hyperactivity disorder:0.62), (VALGANCICLOVIR HYDROCHLORIDE:Attention deficit/hyperactivity disorder:0.7), (YEAST:Attention deficit/hyperactivity disorder:0.34), (ZILEUTON:Attention deficit/hyperactivity disorder:0.47), (PYRAZINAMIDE:Atypical mycobacterial lymphadenitis:0.52), (PYRAZINAMIDE:Atypical mycobacterium test positive:0.55), (VARENICLINE TARTRATE:Autism spectrum disorder:0.17), (AMIODARONE HYDROCHLORIDE:Autoimmune disorder:0.02), (BENAZEPRIL HYDROCHLORIDE:Autoimmune disorder:0.05), (BISOPROLOL FUMARATE:Autoimmune disorder:0.31), (BORTEZOMIB:Autoimmune disorder:0.3), (BUPRENORPHINE HYDROCHLORIDE\NALOXONE HYDROCHLORIDE:Autoimmune disorder:0.29), (CALCIUM CITRATE:Autoimmune disorder:0.14), (CANDESARTAN CILEXETIL:Autoimmune disorder:0.41), (CEPHALEXIN:Autoimmune disorder:0.49), (CLINDAMYCIN\CLINDAMYCIN PHOSPHATE:Autoimmune disorder:0.34), (DACLIZUMAB:Autoimmune disorder:0.56), (DALFAMPRIDINE:Autoimmune disorder:0.33), (DASATINIB:Autoimmune disorder:0.24), (DIMENHYDRINATE:Autoimmune disorder:0.19), (DIVALPROEX SODIUM:Autoimmune disorder:1.25), (ELETRIPTAN HYDROBROMIDE:Autoimmune disorder:0.8), (ENALAPRIL MALEATE:Autoimmune disorder:0.14), (ESZOPICLONE:Autoimmune disorder:0.86), (EVOLOCUMAB:Autoimmune disorder:1.41), (FERROUS SULFATE\FERROUS SULFATE, DRIED:Autoimmune disorder:1.05), (FLUTICASONE:Autoimmune disorder:0.26), (HYDROCHLOROTHIAZIDE\LISINOPRIL:Autoimmune disorder:0.31), (HYDROCHLOROTHIAZIDE\VALSARTAN:Autoimmune disorder:0.05), (HYDROXYUREA:Autoimmune disorder:0.16), (HYDROXYZINE PAMOATE:Autoimmune disorder:0.44), (HYOSCYAMINE:Autoimmune disorder:0.17), (IPRATROPIUM:Autoimmune disorder:0.01), (METAXALONE:Autoimmune disorder:0.57), (METRONIDAZOLE\METRONIDAZOLE HYDROCHLORIDE:Autoimmune disorder:0.28), (NILOTINIB:Autoimmune disorder:0.1), (NORETHINDRONE:Autoimmune disorder:0.08), (OXCARBAZEPINE:Autoimmune disorder:1.14), (PANTOPRAZOLE:Autoimmune disorder:1.26), (PANTOPRAZOLE MAGNESIUM:Autoimmune disorder:0.27), (PEGINTERFERON BETA-1A:Autoimmune disorder:0.02), (PENICILLIN:Autoimmune disorder:0.02), (POLYETHYLENE GLYCOLS:Autoimmune disorder:0.25), (PRAVASTATIN SODIUM:Autoimmune disorder:0.85), (PRIMIDONE:Autoimmune disorder:0.47), (RIOCIGUAT:Autoimmune disorder:0.41), (RISEDRONATE SODIUM:Autoimmune disorder:0.02), (SECUKINUMAB:Autoimmune disorder:0.97), (TERIFLUNOMIDE:Autoimmune disorder:0.73), (TIZANIDINE:Autoimmune disorder:0.24), (UMECLIDINIUM BROMIDE\VILANTEROL TRIFENATATE:Autoimmune disorder:0.16), (AMIODARONE:Autoimmune haemolytic anaemia:0.01), (BUMETANIDE:Autoimmune haemolytic anaemia:0.44), (DILTIAZEM:Autoimmune haemolytic anaemia:0.14), (ENALAPRIL:Autoimmune haemolytic anaemia:0.27), (ILOPROST:Autoimmune haemolytic anaemia:0.02), (NIFEDIPINE:Autoimmune haemolytic anaemia:0.19), (NILOTINIB:Autoimmune haemolytic anaemia:0.07), (PANTOPRAZOLE:Autoimmune haemolytic anaemia:0), (.ALPHA.1-PROTEINASE INHIBITOR HUMAN:Autoimmune thyroiditis:0.02), (ACARBOSE:Autoimmune thyroiditis:0.02), (ACENOCOUMAROL:Autoimmune thyroiditis:0.02), (ACETAZOLAMIDE:Autoimmune thyroiditis:0.25), (ADO-TRASTUZUMAB EMTANSINE:Autoimmune thyroiditis:0.48), (ALCOHOL:Autoimmune thyroiditis:0.43), (AZITHROMYCIN DIHYDRATE:Autoimmune thyroiditis:0.19), (BENAZEPRIL HYDROCHLORIDE:Autoimmune thyroiditis:0.94), (BENAZEPRIL\BENAZEPRIL HYDROCHLORIDE:Autoimmune thyroiditis:0.13), (BENZONATATE:Autoimmune thyroiditis:0.16), (BUDESONIDE\FORMOTEROL FUMARATE:Autoimmune thyroiditis:0.1), (BUPRENORPHINE HYDROCHLORIDE\NALOXONE HYDROCHLORIDE:Autoimmune thyroiditis:0.98), (CALCIPOTRIENE:Autoimmune thyroiditis:0.01), (CALCITONIN SALMON:Autoimmune thyroiditis:0.12), (CALCIUM ACETATE:Autoimmune thyroiditis:0.07), (CALCIUM CARBONATE\VITAMIN D:Autoimmune thyroiditis:0.39), (CANAGLIFLOZIN\METFORMIN HYDROCHLORIDE:Autoimmune thyroiditis:0.03), (CANAKINUMAB:Autoimmune thyroiditis:0.22), (CANDESARTAN CILEXETIL\HYDROCHLOROTHIAZIDE:Autoimmune thyroiditis:1.13), (CARVEDILOL PHOSPHATE:Autoimmune thyroiditis:0.37), (CETIRIZINE HYDROCHLORIDE\PSEUDOEPHEDRINE HYDROCHLORIDE:Autoimmune thyroiditis:0.23), (CLOBAZAM:Autoimmune thyroiditis:0.08), (CLOZAPINE:Autoimmune thyroiditis:1.23), (DASATINIB:Autoimmune thyroiditis:1.34), (DEFLAZACORT:Autoimmune thyroiditis:0.06), (DOCUSATE:Autoimmune thyroiditis:0.5), (DOCUSATE CALCIUM:Autoimmune thyroiditis:0.44), (DOXAZOSIN MESYLATE:Autoimmune thyroiditis:0.45), (DOXAZOSIN\DOXAZOSIN MESYLATE:Autoimmune thyroiditis:0.51), (DOXEPIN HYDROCHLORIDE:Autoimmune thyroiditis:0.23), (DRONABINOL:Autoimmune thyroiditis:0.53), (EDOXABAN TOSYLATE:Autoimmune thyroiditis:0.01), (EMPAGLIFLOZIN\LINAGLIPTIN:Autoimmune thyroiditis:0.18), (ENOXAPARIN:Autoimmune thyroiditis:1.45), (ESTROGENS:Autoimmune thyroiditis:0.07), (FERROUS FUMARATE:Autoimmune thyroiditis:0.14), (FERROUS SULFATE\FERROUS SULFATE, DRIED:Autoimmune thyroiditis:1.58), (FESOTERODINE FUMARATE:Autoimmune thyroiditis:0.51), (GLICLAZIDE:Autoimmune thyroiditis:0.41), (HYDRALAZINE HYDROCHLORIDE:Autoimmune thyroiditis:0.43), (INDAPAMIDE:Autoimmune thyroiditis:0.68), (INSULIN HUMAN:Autoimmune thyroiditis:0.78), (IPRATROPIUM:Autoimmune thyroiditis:0.34), (IRINOTECAN:Autoimmune thyroiditis:0.1), (ISOSORBIDE MONONITRATE:Autoimmune thyroiditis:1.91), (LABETALOL\LABETALOL HYDROCHLORIDE:Autoimmune thyroiditis:0.36), (LEUCOVORIN CALCIUM:Autoimmune thyroiditis:0.49), (LOVASTATIN:Autoimmune thyroiditis:1.5), (METHENAMINE HIPPURATE:Autoimmune thyroiditis:0.17), (MINOCYCLINE\MINOCYCLINE HYDROCHLORIDE:Autoimmune thyroiditis:0.53), (MIRABEGRON:Autoimmune thyroiditis:0.25), (NABUMETONE:Autoimmune thyroiditis:0.31), (NAPROXEN:Autoimmune thyroiditis:2.8), (OXYBUTYNIN CHLORIDE:Autoimmune thyroiditis:0.59), (PAZOPANIB HYDROCHLORIDE:Autoimmune thyroiditis:1.1), (PEGINTERFERON BETA-1A:Autoimmune thyroiditis:0.79), (PEMETREXED DISODIUM:Autoimmune thyroiditis:0.04), (ROFLUMILAST:Autoimmune thyroiditis:0.37), (SACUBITRIL\VALSARTAN:Autoimmune thyroiditis:0.87), (SCOPOLAMINE:Autoimmune thyroiditis:0.79), (SULFAMETHOXAZOLE:Autoimmune thyroiditis:0.26), (TERBUTALINE SULFATE:Autoimmune thyroiditis:0.17), (TETRACYCLINE:Autoimmune thyroiditis:0.3), (TINZAPARIN SODIUM:Autoimmune thyroiditis:0.23), (UMECLIDINIUM BROMIDE\VILANTEROL TRIFENATATE:Autoimmune thyroiditis:0.3), (VEMURAFENIB:Autoimmune thyroiditis:0.03), (VILAZODONE HYDROCHLORIDE:Autoimmune thyroiditis:0.48), (ZINC:Autoimmune thyroiditis:0.42), (CALCIUM CARBONATE\CHOLECALCIFEROL:Autonomic nervous system imbalance:0.08), (DIVALPROEX SODIUM:Autonomic nervous system imbalance:0.59), (ENALAPRIL:Autonomic nervous system imbalance:0.18), (EVOLOCUMAB:Autonomic nervous system imbalance:0.01), (IBRUTINIB:Autonomic nervous system imbalance:0.36), (NAPROXEN:Autonomic nervous system imbalance:0.56), (OXCARBAZEPINE:Autonomic nervous system imbalance:0.5), (PAROXETINE:Autonomic nervous system imbalance:0.09), (SIMVASTATIN:Autonomic nervous system imbalance:0.3), (TAMSULOSIN:Autonomic nervous system imbalance:0.02), (TERIFLUNOMIDE:Autonomic nervous system imbalance:0.13), (VARENICLINE TARTRATE:Autonomic nervous system imbalance:0.53), (BENAZEPRIL HYDROCHLORIDE:Autonomic neuropathy:0.29), (BISOPROLOL FUMARATE:Autonomic neuropathy:0.1), (BUMETANIDE:Autonomic neuropathy:0.41), (CANDESARTAN:Autonomic neuropathy:0.1), (CANDESARTAN CILEXETIL:Autonomic neuropathy:0.31), (CARVEDILOL PHOSPHATE:Autonomic neuropathy:0.12), (CERTOLIZUMAB PEGOL:Autonomic neuropathy:0.53), (CLINDAMYCIN\CLINDAMYCIN PHOSPHATE:Autonomic neuropathy:0.21), (DEVICE:Autonomic neuropathy:0.13), (DOXAZOSIN\DOXAZOSIN MESYLATE:Autonomic neuropathy:0.1), (ENALAPRIL MALEATE:Autonomic neuropathy:0.14), (EVEROLIMUS:Autonomic neuropathy:0.04), (EVOLOCUMAB:Autonomic neuropathy:0.5), (FLUOXETINE\FLUOXETINE HYDROCHLORIDE:Autonomic neuropathy:0.08), (HYDROXYZINE PAMOATE:Autonomic neuropathy:0.44), (HYOSCYAMINE:Autonomic neuropathy:0.19), (IBRUTINIB:Autonomic neuropathy:1.2), (ILOPROST:Autonomic neuropathy:0.28), (IPRATROPIUM:Autonomic neuropathy:0.04), (METAXALONE:Autonomic neuropathy:0.29), (METRONIDAZOLE\METRONIDAZOLE HYDROCHLORIDE:Autonomic neuropathy:0.15), (NEBIVOLOL:Autonomic neuropathy:0.1), (NIFEDIPINE:Autonomic neuropathy:0.15), (NITROFURANTOIN:Autonomic neuropathy:0.16), (OMEGA-3-ACID ETHYL ESTERS:Autonomic neuropathy:0.34), (PHENYTOIN:Autonomic neuropathy:0.26), (PRAMIPEXOLE:Autonomic neuropathy:0.16), (SACUBITRIL\VALSARTAN:Autonomic neuropathy:0.28), (SODIUM BICARBONATE:Autonomic neuropathy:0.24), (SODIUM OXYBATE:Autonomic neuropathy:0.8), (SUMATRIPTAN:Autonomic neuropathy:0.2), (TELMISARTAN:Autonomic neuropathy:0.02), (TERIFLUNOMIDE:Autonomic neuropathy:0.39), (TIMOLOL:Autonomic neuropathy:0.16), (VARENICLINE TARTRATE:Autonomic neuropathy:0.78), (IBRITUMOMAB TIUXETAN:B precursor type acute leukaemia:0.14), (IDARUBICIN HYDROCHLORIDE:B precursor type acute leukaemia:0.32), (THALIDOMIDE:B precursor type acute leukaemia:0.19), (.ALPHA.1-PROTEINASE INHIBITOR HUMAN:B-cell lymphoma:0.35), (ACETAMINOPHEN\PROPOXYPHENE NAPSYLATE:B-cell lymphoma:0.14), (ADO-TRASTUZUMAB EMTANSINE:B-cell lymphoma:0.6), (AGALSIDASE BETA:B-cell lymphoma:0.37), (ALCOHOL:B-cell lymphoma:0.22), (ALUMINUM HYDROXIDE\MAGNESIUM OXIDE\OXETHAZAINE:B-cell lymphoma:0.29), (AMANTADINE:B-cell lymphoma:0.31), (AMILORIDE HYDROCHLORIDE\HYDROCHLOROTHIAZIDE:B-cell lymphoma:0.27), (AMLODIPINE BESYLATE\OLMESARTAN MEDOXOMIL:B-cell lymphoma:0.04), (AMMONIUM LACTATE:B-cell lymphoma:0.21), (AMPICILLIN SODIUM\SULBACTAM SODIUM:B-cell lymphoma:0.25), (ANIDULAFUNGIN:B-cell lymphoma:0.13),(ARMODAFINIL:B-cell lymphoma:0.59), (ATROPINE SULFATE:B-cell lymphoma:0.09), (BENAZEPRIL\BENAZEPRIL HYDROCHLORIDE:B-cell lymphoma:0.64), (BETAMETHASONE DIPROPIONATE:B-cell lymphoma:0.3), (BRINZOLAMIDE:B-cell lymphoma:0.46), (BUPRENORPHINE HYDROCHLORIDE\NALOXONE HYDROCHLORIDE:B-cell lymphoma:0.37), (CABAZITAXEL:B-cell lymphoma:0.02), (CALCITONIN SALMON:B-cell lymphoma:0.72), (CANAKINUMAB:B-cell lymphoma:0.31), (CARISOPRODOL:B-cell lymphoma:0.37), (CASPOFUNGIN ACETATE:B-cell lymphoma:0.2), (CEFOTAXIME\CEFOTAXIME SODIUM:B-cell lymphoma:0.18), (CERITINIB:B-cell lymphoma:0.11), (CERTOLIZUMAB PEGOL:B-cell lymphoma:1.51), (CLINDAMYCIN HYDROCHLORIDE:B-cell lymphoma:0.15), (CLOFARABINE:B-cell lymphoma:0.17), (COLESEVELAM HYDROCHLORIDE:B-cell lymphoma:0.16), (COLISTIMETHATE SODIUM:B-cell lymphoma:0.06), (CYPROHEPTADINE:B-cell lymphoma:0.37), (DABIGATRAN:B-cell lymphoma:0.87), (DABRAFENIB:B-cell lymphoma:0.77), (DACLIZUMAB:B-cell lymphoma:1.15), (DARATUMUMAB:B-cell lymphoma:0.47), (DASATINIB:B-cell lymphoma:1.9), (DECITABINE:B-cell lymphoma:0.46), (DESFLURANE:B-cell lymphoma:0.06), (DIPYRIDAMOLE:B-cell lymphoma:0.4), (DOFETILIDE:B-cell lymphoma:0.58), (DOXEPIN:B-cell lymphoma:0.33), (DOXEPIN HYDROCHLORIDE:B-cell lymphoma:0.01), (DOXYCYCLINE HYCLATE:B-cell lymphoma:0.2), (DRONEDARONE:B-cell lymphoma:0.11), (ELETRIPTAN HYDROBROMIDE:B-cell lymphoma:0.79), (ELOTUZUMAB:B-cell lymphoma:0.87), (ESTROGENS, CONJUGATED\MEDROXYPROGESTERONE ACETATE:B-cell lymphoma:0.42), (ETORICOXIB:B-cell lymphoma:0.4), (EVOLOCUMAB:B-cell lymphoma:0.31), (EXEMESTANE:B-cell lymphoma:0.11), (FESOTERODINE FUMARATE:B-cell lymphoma:1.22), (FORMOTEROL FUMARATE:B-cell lymphoma:0.93), (FORMOTEROL FUMARATE\MOMETASONE FUROATE:B-cell lymphoma:0.26), (GIMERACIL\OTERACIL\TEGAFUR:B-cell lymphoma:0.44), (GLYCOPYRROLATE:B-cell lymphoma:0.35), (GLYCOPYRRONIUM\INDACATEROL:B-cell lymphoma:0.97), (HEPARIN CALCIUM:B-cell lymphoma:0.83), (HERBALS\TURMERIC:B-cell lymphoma:0.08), (HYDROCORTISONE ACETATE:B-cell lymphoma:0.13), (HYDROXYZINE PAMOATE:B-cell lymphoma:1.21), (HYOSCYAMINE:B-cell lymphoma:0.26), (IDARUBICIN:B-cell lymphoma:1.03), (IDARUBICIN HYDROCHLORIDE:B-cell lymphoma:0.19), (ILOPROST:B-cell lymphoma:0.2), (IMATINIB MESYLATE:B-cell lymphoma:1.11), (IMIPRAMINE HYDROCHLORIDE:B-cell lymphoma:0.39), (IMMUNE GLOBULIN NOS:B-cell lymphoma:0.07), (INDAPAMIDE\PERINDOPRIL:B-cell lymphoma:1.84), (INDOMETHACIN:B-cell lymphoma:0.65), (INTERFERON GAMMA-1B:B-cell lymphoma:0.11), (IRINOTECAN:B-cell lymphoma:0.61), (LABETALOL HYDROCHLORIDE:B-cell lymphoma:0.58), (LACTOBACILLUS RHAMNOSUS:B-cell lymphoma:0.29), (LAPATINIB DITOSYLATE:B-cell lymphoma:0.07), (LEDIPASVIR\SOFOSBUVIR:B-cell lymphoma:0.9), (LISDEXAMFETAMINE DIMESYLATE:B-cell lymphoma:0.07), (MAGNESIUM ASPARTATE\POTASSIUM ASPARTATE:B-cell lymphoma:0.2), (METHENAMINE HIPPURATE:B-cell lymphoma:0.77), (METHENOLONE ENANTHATE:B-cell lymphoma:0.09), (MIDODRINE:B-cell lymphoma:0.44), (MINOCYCLINE HYDROCHLORIDE:B-cell lymphoma:0.46), (MODAFINIL:B-cell lymphoma:0.42), (MUPIROCIN\MUPIROCIN CALCIUM:B-cell lymphoma:0.43), (MYCOPHENOLATE MOFETIL\MYCOPHENOLATE MOFETIL HYDROCHLORIDE:B-cell lymphoma:1.07), (NATEGLINIDE:B-cell lymphoma:0.18), (NICARDIPINE:B-cell lymphoma:0.45), (NINTEDANIB:B-cell lymphoma:0.37), (OLAPARIB:B-cell lymphoma:0.04), (OLODATEROL HYDROCHLORIDE\TIOTROPIUM BROMIDE MONOHYDRATE:B-cell lymphoma:0.34), (OXCARBAZEPINE:B-cell lymphoma:1.64), (PANITUMUMAB:B-cell lymphoma:0.09), (PAZOPANIB HYDROCHLORIDE:B-cell lymphoma:1.06), (PEMETREXED DISODIUM:B-cell lymphoma:0.73), (PHENOBARBITAL:B-cell lymphoma:0.87), (PHENYTOIN SODIUM:B-cell lymphoma:0.32), (PONATINIB:B-cell lymphoma:1.51), (PROPAFENONE HYDROCHLORIDE:B-cell lymphoma:0.1), (REGORAFENIB:B-cell lymphoma:0.66), (RIVASTIGMINE TARTRATE:B-cell lymphoma:0.34), (ROPINIROLE:B-cell lymphoma:0.38), (SACUBITRIL\VALSARTAN:B-cell lymphoma:0.87), (SALMETEROL XINAFOATE:B-cell lymphoma:0.07), (SECUKINUMAB:B-cell lymphoma:0.74), (SODIUM OXYBATE:B-cell lymphoma:0.58), (SUNITINIB MALATE:B-cell lymphoma:0.32), (TERBUTALINE:B-cell lymphoma:0.3), (TERBUTALINE SULFATE:B-cell lymphoma:1.07), (TIGECYCLINE:B-cell lymphoma:0.67), (TOPIRAMATE:B-cell lymphoma:1.62), (TRAMETINIB:B-cell lymphoma:0.75), (TRANDOLAPRIL:B-cell lymphoma:0.09), (TRETINOIN:B-cell lymphoma:0.06), (TRIAMTERENE:B-cell lymphoma:0.16), (UMECLIDINIUM BROMIDE:B-cell lymphoma:0.06), (UMECLIDINIUM BROMIDE\VILANTEROL TRIFENATATE:B-cell lymphoma:0.97), (VALGANCICLOVIR HYDROCHLORIDE:B-cell lymphoma:1.57), (VARENICLINE TARTRATE:B-cell lymphoma:0.94), (VEMURAFENIB:B-cell lymphoma:0.44), (ZIV-AFLIBERCEPT:B-cell lymphoma:0.03), (VINCRISTINE SULFATE:B-cell lymphoma recurrent:0.14), (ACETAMINOPHEN\CODEINE PHOSPHATE:B-cell small lymphocytic lymphoma:0.33), (ALBUTEROL SULFATE\IPRATROPIUM BROMIDE:B-cell small lymphocytic lymphoma:0.03), (AMIODARONE HYDROCHLORIDE:B-cell small lymphocytic lymphoma:0.06), (AMPHETAMINE ASPARTATE\AMPHETAMINE SULFATE\DEXTROAMPHETAMINE SACCHARATE\DEXTROAMPHETAMINE SULFATE:B-cell small lymphocytic lymphoma:0.07), (CALCIUM ACETATE:B-cell small lymphocytic lymphoma:0.01), (CANDESARTAN CILEXETIL:B-cell small lymphocytic lymphoma:0.32), (CERTOLIZUMAB PEGOL:B-cell small lymphocytic lymphoma:0.23), (CHOLESTYRAMINE:B-cell small lymphocytic lymphoma:0.29), (CLINDAMYCIN\CLINDAMYCIN PHOSPHATE:B-cell small lymphocytic lymphoma:0.39), (DASATINIB:B-cell small lymphocytic lymphoma:0.09), (DEXLANSOPRAZOLE:B-cell small lymphocytic lymphoma:0.79), (DIPHENHYDRAMINE:B-cell small lymphocytic lymphoma:0.36), (ENOXAPARIN:B-cell small lymphocytic lymphoma:0.15), (EVOLOCUMAB:B-cell small lymphocytic lymphoma:0.23), (FENOFIBRATE:B-cell small lymphocytic lymphoma:0.15), (FERROUS SULFATE\FERROUS SULFATE, DRIED:B-cell small lymphocytic lymphoma:0.6), (METAXALONE:B-cell small lymphocytic lymphoma:0.04), (METRONIDAZOLE:B-cell small lymphocytic lymphoma:1.03), (NEBIVOLOL:B-cell small lymphocytic lymphoma:0.02), (NILOTINIB:B-cell small lymphocytic lymphoma:0.09), (NYSTATIN:B-cell small lymphocytic lymphoma:0.17), (OXYGEN:B-cell small lymphocytic lymphoma:0.23), (RANOLAZINE:B-cell small lymphocytic lymphoma:0.28), (SODIUM OXYBATE:B-cell small lymphocytic lymphoma:0.03), (TERIFLUNOMIDE:B-cell small lymphocytic lymphoma:0.11), (THALIDOMIDE:B-cell small lymphocytic lymphoma:0.64), (TRIAMCINOLONE ACETONIDE:B-cell small lymphocytic lymphoma:0.02), (UNSPECIFIED INGREDIENT:B-cell small lymphocytic lymphoma:1.29), (VERAPAMIL HYDROCHLORIDE:B-cell small lymphocytic lymphoma:0.67), (CLADRIBINE:B-cell type acute leukaemia:0.1), (IBRITUMOMAB TIUXETAN:B-cell type acute leukaemia:0.11), (THIOGUANINE:B-cell type acute leukaemia:0.14), (.ALPHA.1-PROTEINASE INHIBITOR HUMAN:Back disorder:0.08), (ALBIGLUTIDE:Back disorder:0.33), (ALCOHOL:Back disorder:0.13), (BISACODYL:Back disorder:0.01), (BISOPROLOL:Back disorder:0.23), (BISOPROLOL FUMARATE:Back disorder:0.15), (BUDESONIDE\FORMOTEROL FUMARATE:Back disorder:0.11), (BUMETANIDE:Back disorder:0.53), (CALCIUM ACETATE:Back disorder:0.06), (CALCIUM CARBONATE\CHOLECALCIFEROL:Back disorder:0.7), (CANDESARTAN:Back disorder:0.31), (CANDESARTAN CILEXETIL\HYDROCHLOROTHIAZIDE:Back disorder:0.12), (CARVEDILOL PHOSPHATE:Back disorder:0.54), (CEPHALEXIN:Back disorder:0.87), (CERTOLIZUMAB PEGOL:Back disorder:1.25), (CETIRIZINE HYDROCHLORIDE\PSEUDOEPHEDRINE HYDROCHLORIDE:Back disorder:0.15), (CHLORTHALIDONE:Back disorder:0.36), (CIMETIDINE:Back disorder:0.18), (CIPROFLOXACIN:Back disorder:0.05), (CLINDAMYCIN\CLINDAMYCIN PHOSPHATE:Back disorder:0.37), (CLOTRIMAZOLE:Back disorder:0.28), (CLOZAPINE:Back disorder:0.15), (COD LIVER OIL:Back disorder:0.12), (CORTICOTROPIN:Back disorder:0.08), (DACLIZUMAB:Back disorder:0.22), (DAPAGLIFLOZIN PROPANEDIOL:Back disorder:0.2), (DOXEPIN:Back disorder:0.01), (DOXEPIN HYDROCHLORIDE:Back disorder:0.24), (ESOMEPRAZOLE:Back disorder:0.02), (ESTROGENS:Back disorder:0.07), (EVEROLIMUS:Back disorder:0.39), (EVOLOCUMAB:Back disorder:1.34), (FESOTERODINE FUMARATE:Back disorder:0.26), (FLECAINIDE:Back disorder:0.37), (GLYBURIDE:Back disorder:0.43), (HEPARIN SODIUM:Back disorder:0.18), (HYDROXYCHLOROQUINE DIPHOSPHATE:Back disorder:0.13), (HYDROXYUREA:Back disorder:0.4), (HYDROXYZINE PAMOATE:Back disorder:1.54), (IBRUTINIB:Back disorder:0.62), (ILOPROST:Back disorder:0.65), (IMIPRAMINE HYDROCHLORIDE:Back disorder:0.07), (LEVALBUTEROL HYDROCHLORIDE:Back disorder:0.88), (LEVOMEFOLATE CALCIUM:Back disorder:0.06), (LINAGLIPTIN:Back disorder:0.15), (LOMITAPIDE MESYLATE:Back disorder:0.12), (MECLIZINE\MECLIZINE HYDROCHLORIDE:Back disorder:0.02), (METHENAMINE HIPPURATE:Back disorder:0.37), (METHYLPREDNISOLONE ACETATE:Back disorder:0.64), (METHYLPREDNISOLONE SODIUM SUCCINATE:Back disorder:0.48), (MOXIFLOXACIN HYDROCHLORIDE:Back disorder:0.59), (NADOLOL:Back disorder:0.19), (PEGINTERFERON BETA-1A:Back disorder:0.53), (PHENOBARBITAL:Back disorder:0.75), (PRAZOSIN:Back disorder:0.1), (PROPAFENONE HYDROCHLORIDE:Back disorder:0.04), (RABEPRAZOLE SODIUM:Back disorder:0.34), (RALOXIFENE HYDROCHLORIDE:Back disorder:0.72), (SCOPOLAMINE:Back disorder:0.14), (SELEXIPAG:Back disorder:0.38), (SOLIFENACIN SUCCINATE:Back disorder:0.07), (TERBUTALINE SULFATE:Back disorder:0.19), (TETRACYCLINE:Back disorder:0.37), (THYROID, PORCINE:Back disorder:0.34), (TIMOLOL:Back disorder:0.16), (TOCILIZUMAB:Back disorder:0.4), (UMECLIDINIUM BROMIDE\VILANTEROL TRIFENATATE:Back disorder:0.88), (VITAMIN A:Back disorder:0.14), (ZINC:Back disorder:0.27), (ZIPRASIDONE HYDROCHLORIDE\ZIPRASIDONE MESYLATE:Back disorder:0.07), (ZONISAMIDE:Back disorder:0.22), (ZOPICLONE:Back disorder:0), (CALCIUM:Back injury:0.9), (DEXLANSOPRAZOLE:Back injury:0.57), (EVOLOCUMAB:Back injury:0.37), (OLANZAPINE:Back injury:0.12), (PANTOPRAZOLE:Back injury:0.13), (POTASSIUM:Back injury:0.12), (RAMIPRIL:Back injury:0.03), (SUMATRIPTAN\SUMATRIPTAN SUCCINATE:Back injury:0.01), (TERIFLUNOMIDE:Back injury:0.13), (VARENICLINE TARTRATE:Back injury:0.27), (.ALPHA.-TOCOPHEROL\VITAMINS:Back pain:0.57), (.BETA.-ACETYLDIGOXIN:Back pain:0.1), (.BETA.-CAROTENE:Back pain:0.1), (.BETA.-HYDROXYISOVALERIC ACID\ARGININE\GLUTAMINE:Back pain:0.25), (ABOBOTULINUMTOXINA:Back pain:0.79), (ACAMPROSATE:Back pain:0.23), (ACETAMINOPHEN\BROMISOVAL\DIHYDROCODEINE PHOSPHATE\DIPHENHYDRAMINE SALICYLATE\DYPHYLLINE\METHYLEPHEDRINE HYDROCHLORIDE, (+/−)-:Back pain:0.13), (ACETAMINOPHEN\CHLORPHENIRAMINE MALEATE\PSEUDOEPHEDRINE HYDROCHLORIDE:Back pain:0.11), (ACRIVASTINE\PSEUDOEPHEDRINE HYDROCHLORIDE:Back pain:0.35), (ADENINE:Back pain:1.25), (AGOMELATINE:Back pain:0.07), (ALBUTEROL\IPRATROPIUM BROMIDE:Back pain:0.27), (ALCLOMETASONE:Back pain:0.11), (ALDESLEUKIN:Back pain:0.73), (ALFALFA:Back pain:0.08), (ALISKIREN HEMIFUMARATE\HYDROCHLOROTHIAZIDE:Back pain:0.61), (ALISKIREN HEMIFUMARATE\VALSARTAN:Back pain:0.2), (ALUMINUM HYDROXIDE\MAGNESIUM OXIDE\OXETHAZAINE:Back pain:0.36), (ALUMINUM SILICATE:Back pain:0.85), (AMILORIDE HYDROCHLORIDE\FUROSEMIDE:Back pain:0.56), (AMINO ACIDS\DEXTROSE\ELECTROLYTES NOS:Back pain:2.39), (AMINOBENZOIC ACID\CALCIUM PANTOTHENATE\CYSTINE\KERATIN\THIAMINE MONONITRATE:Back pain:0.1), (AMINOCAPROIC ACID:Back pain:0.01), (AMINOSALICYLIC ACID:Back pain:0.27), (AMMONIUM CHLORIDE\DEXTROMETHORPHAN\SODIUM CITRATE:Back pain:1.1), (AMOBARBITAL SODIUM:Back pain:0.07), (AMOXAPINE:Back pain:1.64), (AMPHETAMINE\DEXTROAMPHETAMINE:Back pain:0.07), (AMPICILLIN SODIUM:Back pain:1.34), (ANAGLIPTIN:Back pain:0.98), (ANAGRELIDE HYDROCHLORIDE:Back pain:1.27), (ANAKINRA:Back pain:0.58), (ANTIHISTAMINES NOS:Back pain:1.71), (ASCORBIC ACID/CHROMIUM/SELENIUM/VANADYL SULFATE:Back pain:0.84), (ASCORBIC ACID\BIOTIN\CALCIUM PANTOTHENATE\CYANOCOBALAMIN\FOLIC ACID\NIACINAMIDE\PYRIDOXINE HYDROCHLORIDE\RIBOFLAVIN\THIAMINE HYDROCHLORIDE:Back pain:0.08), (ASCORBIC ACID\FERROUS SULFATE\FOLIC ACID\PYRIDOXINE HYDROCHLORIDE\RIBOFLAVIN\THIAMINE MONONITRATE:Back pain:0.15), (ASPIRIN\CAFFEINE\SALICYLAMIDE:Back pain:0.21), (ASPIRIN\DIHYDROXYALUMINUM AMINOACETATE\MAGNESIUM CARBONATE:Back pain:0.02), (ASUNAPREVIR:Back pain:1.09), (ATORVASTATIN CALCIUM\EZETIMIBE:Back pain:0.36), (ATOVAQUONE\PROGUANIL HYDROCHLORIDE:Back pain:0), (ATRACURIUM:Back pain:0.4), (ATRACURIUM BESYLATE:Back pain:0.41), (AZITHROMYCIN OR METHOTREXATE SODIUM:Back pain:0.88), (BACITRACIN\NEOMYCIN SULFATE:Back pain:0.19), (BAMIFYLLINE HYDROCHLORIDE:Back pain:0.96), (BENPROPERINE PHOSPHATE:Back pain:0.26), (BENZOCAINE\RESORCINOL:Back pain:0.08), (BEPOTASTINE BESILATE:Back pain:0.73), (BEPRIDIL:Back pain:2.55), (BERAPROST:Back pain:1.07), (BETAMETHASONE DIPROPIONATE\CALCIPOTRIENE HYDRATE:Back pain:0.65), (BETAMETHASONE DIPROPIONATE\GENTAMICIN SULFATE:Back pain:0.11), (BETAMETHASONE SODIUM PHOSPHATE\NEOMYCIN SULFATE:Back pain:0.5), (BETAMIPRON\PANIPENEM:Back pain:0.19), (BEXAROTENE:Back pain:0.12), (BIFIDOBACTERIUM LONGUM\LACTOBACILLUS ACIDOPHILUS:Back pain:0.24), (BLONANSERIN:Back pain:0.76), (BORIC ACID:Back pain:0.27), (BORON:Back pain:0.32), (BOTULINUM TOXIN TYPE A:Back pain:0.87), (BROMPERIDOL:Back pain:0.27), (BUFORMIN:Back pain:0.36), (BUPROPION HYDROBROMIDE:Back pain:0.04), (CABOZANTINIB:Back pain:0.82), (CAJUPUT OIL\EUCALYPTUS OIL\PEPPERMINT OIL:Back pain:0.36), (CALCIUM BICARBONATE:Back pain:0.47), (CALCIUM CARBONATE\ETIDRONATE DISODIUM:Back pain:0.65), (CALCIUM CHLORIDE\ICODEXTRIN\MAGNESIUM CHLORIDE\SODIUM CHLORIDE\SODIUM LACTATE:Back pain:0.04), (CALCIUM PANTOTHENATE\CYANOCOBALAMIN\NIACINAMIDE\PYRIDOXINE HYDROCHLORIDE\RIBOFLAVIN\THIAMINE MONONITRATE:Back pain:1), (CALCIUM PHOSPHATE:Back pain:0.16), (CALCIUM PHOSPHATE, DIBASIC, ANHYDROUS\FERROUS SUCCINATE\MAGNESIUM HYDROXIDE:Back pain:0.21), (CARBAMIDE PEROXIDE:Back pain:0.56), (CARBOXYMETHYLCELLULOSE SODIUM\GLYCERIN\POLYSORBATE 80:Back pain:0.98), (CARMUSTINE:Back pain:0.28), (CARTEOLOL:Back pain:0.16), (CEFOTAXIME\CEFOTAXIME SODIUM:Back pain:1.39), (CEFOTIAM HYDROCHLORIDE:Back pain:0.28), (CEFPROZIL:Back pain:1.1), (CEFTERAM PIVOXIL:Back pain:0.44), (CERIVASTATIN SODIUM:Back pain:1.1), (CETRAXATE HYDROCHLORIDE:Back pain:0.06), (CETYLPYRIDINIUM CHLORIDE OR SODIUM FLUORIDE OR SODIUM MONOFLUOROPHOSPHATE OR GLUCOSE OXIDASE\LACTOFERRIN\LACTOPEROXIDASE BOVINE\LYSOZYME:Back pain:0.4), (CHLORAL HYDRATE:Back pain:0.24), (CHLORMADINONE ACETATE\ETHINYL ESTRADIOL:Back pain:0.28), (CHLORPROMAZINE HYDROCHLORIDE:Back pain:0.25), (CHLORPROMAZINE\PROMETHAZINE:Back pain:0.16), (CHLORPROTHIXENE:Back pain:0.86), (CHOLINE ALFOSCERATE:Back pain:0.24), (CHOLINE BITARTRATE\CYSTEINE\INOSITOL\MAMMAL LIVER:Back pain:0.49), (CHONDROITIN SULFATE (CHICKEN):Back pain:1.22), (CICLOPIROX OLAMINE:Back pain:0.74), (CIFENLINE:Back pain:1.19), (CIPROFLOXACIN\DEXAMETHASONE:Back pain:0.17), (CITICOLINE:Back pain:0.31), (CLENBUTEROL HYDROCHLORIDE:Back pain:1.34), (CLIKSTAR:Back pain:0.98), (CLOSTRIDIUM TETANI TOXOID ANTIGEN (FORMALDEHYDE INACTIVATED):Back pain:0.14), (CLOXAZOLAM:Back pain:1.21), (COBICISTAT\DARUNAVIR ETHANOLATE:Back pain:0.13), (COBICISTAT\ELVITEGRAVIR\EMTRICITABINE\TENOFOVIR:Back pain:0.08), (CODEINE PHOSPHATE\PROMETHAZINE:Back pain:0.22), (COLISTIMETHATE SODIUM:Back pain:0.96), (CROTALIDAE POLYVALENT IMMUNE FAB (OVINE):Back pain:0.29), (CYANOCOBALAMIN CO-57\FOLIC ACID\PYRIDOXINE HYDROCHLORIDE:Back pain:0.06), (CYANOCOBALAMIN\PYRIDOXINE HYDROCHLORIDE\RIBOFLAVIN\SODIUM PANTOTHENATE\THIAMINE HYDROCHLORIDE\VITAMIN B COMPLEX:Back pain:0.03), (CYCLOPENTOLATE:Back pain:0.61), (CYPROTERONE:Back pain:0.21), (CYPROTERONE ACETATE\ETHINYL ESTRADIOL:Back pain:0.96), (CYSTEINE:Back pain:0.04), (DANTROLENE SODIUM:Back pain:1.72), (DARIFENACIN\DARIFENACIN HYDROBROMIDE:Back pain:0.17), (DARUNAVIR\RITONAVIR:Back pain:0.37), (DEFERIPRONE:Back pain:1.03), (DEHYDROEPIANDROSTERONE-3-SULFATE SODIUM:Back pain:0.27), (DEMECLOCYCLINE:Back pain:0.02), (DIASTASE:Back pain:0.04), (DIGITALIS:Back pain:0.41), (DIMETHICONE 410:Back pain:0.54), (DIMETHYL SULFOXIDE:Back pain:0.42), (DOCUSATE SODIUM\SENNOSIDE A\SENNOSIDE B:Back pain:0.01), (DYDROGESTERONE:Back pain:0.1), (ECALLANTIDE:Back pain:0.23), (EFLORNITHINE HYDROCHLORIDE:Back pain:0.06), (ELBASVIR\GRAZOPREVIR:Back pain:0.39), (EMTRICITABINE\TENOFOVIR ALAFENAMIDE FUMARATE:Back pain:0.01), (EPHEDRINE HYDROCHLORIDE:Back pain:0.82), (EPHEDRINE HYDROCHLORIDE\GUAIFENESIN:Back pain:0.2), (EPINEPHRINE\LIDOCAINE:Back pain:0.15), (EPRAZINONE:Back pain:0.61), (ERGOLOID MESYLATES:Back pain:0.24), (ERIBULIN MESYLATE:Back pain:0.96), (ERYTHROMYCIN LACTOBIONATE:Back pain:0.66), (ERYTHROMYCIN\SULFISOXAZOLE:Back pain:0.72), (ESTRADIOL ACETATE:Back pain:0.54), (ESTRADIOL BENZOATE:Back pain:0.68), (ESTRADIOL\LEVONORGESTREL:Back pain:0.55), (ESTROGENS\METHYLTESTOSTERONE:Back pain:0.19), (ETHACRYNIC ACID:Back pain:1.3), (ETHINYL ESTRADIOL:Back pain:0.03), (ETHINYL ESTRADIOL\ETHYNODIOL DIACETATE:Back pain:0.14), (ETHINYL ESTRADIOL\FERROUS FUMARATE\NORETHINDRONE:Back pain:0.21), (ETOMIDATE:Back pain:0.41), (ETRETINATE:Back pain:2.84), (EUCALYPTOL\MENTHOL\METHYL SALICYLATE\THYMOL:Back pain:0.76), (FELBAMATE:Back pain:0.43), (FELODIPINE\METOPROLOL SUCCINATE:Back pain:0.27), (FENOPROFEN CALCIUM:Back pain:0.24), (FERRIC SODIUM CITRATE:Back pain:0.31), (FERROUS BISGLYCINATE:Back pain:1.37), (FERROUS GLYCINE SULFATE:Back pain:0.5), (FEXOFENADINE HYDROCHLORIDE\PSEUDOEPHEDRINE HYDROCHLORIDE:Back pain:0.14), (FIBRINOGEN HUMAN:Back pain:0.16), (FLAVIN ADENINE DINUCLEOTIDE:Back pain:1.67), (FLOMOXEF:Back pain:0.5), (FLUNARIZINE HYDROCHLORIDE:Back pain:0.96), (FLUPENTIXOL:Back pain:0.18), (FLUPHENAZINE:Back pain:1.05), (FLUPHENAZINE HYDROCHLORIDE:Back pain:0.1), (FLURANDRENOLIDE:Back pain:0.41), (FOSFOMYCIN CALCIUM:Back pain:0.55), (FOSINOPRIL SODIUM\HYDROCHLOROTHIAZIDE:Back pain:0.15), (FURSULTIAMINE:Back pain:1.22), (FUSIDATE SODIUM:Back pain:0.05), (GADOBENATE DIMEGLUMINE:Back pain:0.94), (GADOTERATE MEGLUMINE:Back pain:0.17), (GADOVERSETAMIDE:Back pain:0.86), (GALANTAMINE HYDROBROMIDE:Back pain:0.09), (GELATIN:Back pain:0.17), (GENTIAN VIOLET:Back pain:0.12), (GLIMEPIRIDE\METFORMIN:Back pain:0.39), (GLIPIZIDE\METFORMIN HYDROCHLORIDE:Back pain:1.11), (GLUTAMINE\SODIUM GUALENATE:Back pain:0.41), (GLYCOPYRROLATE\INDACATEROL MALEATE:Back pain:1.44), (GOLD:Back pain:0.11), (GREEN TEA LEAF:Back pain:0.02), (HALOPERIDOL DECANOATE:Back pain:0.98), (HERBALS\MINERALS\VITAMINS:Back pain:0.45), (HORSE CHESTNUT:Back pain:0.81), (HYDRALAZINE HYDROCHLORIDE\ISOSORBIDE DINITRATE:Back pain:0.11), (HYDRAZINE SULFATE:Back pain:0.25), (HYDROCHLORIC ACID:Back pain:0), (HYDROCHLOROTHIAZIDE\QUINAPRIL HYDROCHLORIDE:Back pain:1), (HYDROCORTISONE ACETATE\PRAMOXINE HYDROCHLORIDE:Back pain:0.25), (HYDROCORTISONE PROBUTATE:Back pain:0.4), (HYDROQUINONE:Back pain:0.43), (HYDROXYCITRIC ACID:Back pain:0.35), (HYOSCYAMINE SULFATE\METHENAMINE\METHYLENE BLUE\PHENYL SALICYLATE\SODIUM PHOSPHATE, MONOBASIC, MONOHYDRATE:Back pain:1.08), (HYPERICUM PERFORATUM:Back pain:0.06), (IBRITUMOMAB TIUXETAN:Back pain:1.01), (IFENPRODIL:Back pain:0.98), (IFENPRODIL TARTRATE:Back pain:0.17), (ILOPROST:Back pain:1.57), (IMIDAPRIL HYDROCHLORIDE:Back pain:0.46), (IMIGLUCERASE:Back pain:1.69), (IMIPRAMINE PAMOATE:Back pain:0.08), (IMMUNE GLOBULIN NOS:Back pain:1.34), (INFLIXIMAB-DYYB:Back pain:1.77), (INFLUENZA A VIRUS A/CALIFORNIA/7/2009 X-179A (H1N1) ANTIGEN (FORMALDEHYDE INACTIVATED)\INFLUENZA A VIRUS A/VICTORIA/210/2009 X-187 (H3N2) ANTIGEN (FORMALDEHYDE INACTIVATED)\INFLUENZA B VIRUS B/BRISBANE/60/2008 ANTIGEN (FORMALDEHYDE INACTIVATED):Back pain:0.2), (INFLUENZA VIRUS VACCINE TRIVALENT TYPE A+B AFLURIA:Back pain:0.26), (INSULIN ASPART\INSULIN DEGLUDEC:Back pain:0.38), (INSULIN PORK\INSULIN PURIFIED PORK:Back pain:0.78), (INTERFERON ALFA-2A:Back pain:1.79), (INTERFERON ALFA-2A OR INTERFERON ALFA-2B:Back pain:0.48), (INTERFERON ALFA-N1:Back pain:0), (INTERFERON BETA:Back pain:0.59), (INTERFERON GAMMA-1B:Back pain:1.12), (IODINE\POTASSIUM IODIDE:Back pain:0.03), (IRBESARTAN\TRICHLORMETHIAZIDE:Back pain:0.1), (ISOFLURANE:Back pain:0.41), (ISONIAZID\RIFAMPIN:Back pain:1.69), (ITOPRIDE:Back pain:0.94), (IVABRADINE HYDROCHLORIDE:Back pain:0.45), (IXABEPILONE:Back pain:0.28), (IXEKIZUMAB:Back pain:0.23), (LACTOBACILLUS ACIDOPHILUS\LACTOBACILLUS RHAMNOSUS:Back pain:0.14), (LANDIOLOL HYDROCHLORIDE:Back pain:0.9), (LATANOPROST\TIMOLOL:Back pain:0.6), (LEVALBUTEROL:Back pain:1.48), (LEVOMEPROMAZINE MALEATE:Back pain:0.1), (LEVOSIMENDAN:Back pain:0.54), (LICORICE ROOT EXTRACT\RHUBARB:Back pain:0.1), (LINSIDOMINE:Back pain:0.76), (LOMUSTINE:Back pain:0.1), (LOVASTATIN\NIACIN:Back pain:0.13), (LUMACAFTOR:Back pain:0.16), (LURASIDONE:Back pain:0.04), (LUTETIUM LU-177:Back pain:0.96), (LYCOPENE:Back pain:0.05), (MAGNESIUM PIDOLATE:Back pain:0.42), (MAGNESIUM SULFATE\POTASSIUM SULFATE\SODIUM SULFATE:Back pain:0.02), (MAGNESIUM\PYRIDOXINE:Back pain:0.46), (MECLOZINE DIHYDROCHLORIDE:Back pain:0.21), (MELPERONE:Back pain:1.88), (MEPENZOLATE BROMIDE:Back pain:0.59), (MEPOLIZUMAB:Back pain:1.85), (MEQUITAZINE:Back pain:0.27), (MESTRANOL\NORETHINDRONE:Back pain:0.49), (METFORMIN HYDROCHLORIDE\SAXAGLIPTIN:Back pain:0.98), (METHENAMINE MANDELATE:Back pain:1.02), (METHYLENE BLUE:Back pain:0.62), (METRELEPTIN:Back pain:0.41), (MIGLUSTAT:Back pain:0.79), (MILRINONE LACTATE:Back pain:0.05), (MOEXIPRIL HYDROCHLORIDE:Back pain:1.98), (MOSAPRIDE CITRATE DIHYDRATE:Back pain:1.01), (NAFAMOSTAT MESYLATE:Back pain:0.49), (NAFTIFINE HYDROCHLORIDE:Back pain:0.26), (NALFURAFINE HYDROCHLORIDE:Back pain:0.06), (NEOMYCIN:Back pain:1.07), (NEOSTIGMINE METHYLSULFATE:Back pain:0.22), (NILVADIPINE:Back pain:0.25), (NUSINERSEN:Back pain:0.14), (OK-432:Back pain:0.15), (OLANZAPINE PAMOATE:Back pain:0.42), (OLODATEROL HYDROCHLORIDE:Back pain:0.22), (OXYBUTYNIN\OXYBUTYNIN CHLORIDE:Back pain:1.57), (PAPAYA:Back pain:0.45), (PAZUFLOXACIN MESILATE:Back pain:0.36), (PENICILLAMINE:Back pain:0.5), (PENTOSTATIN:Back pain:1.23), (PEROSPIRONE:Back pain:0.55), (PHENELZINE:Back pain:0.59), (PHENYLEPHRINE HYDROCHLORIDE\WITCH HAZEL:Back pain:0.29), (PHOSPHORUS:Back pain:0.32), (PIMOBENDAN:Back pain:2.16), (PINDOLOL:Back pain:0.55), (PIPERACILLIN SODIUM:Back pain:0.27), (PIRENZEPINE:Back pain:0.23), (PIZOTYLINE:Back pain:0.79), (PODOFILOX:Back pain:0.35), (POLYMYXIN B SULFATE:Back pain:0.I8), (POLYSTYRENE SULFONIC ACID:Back pain:0.77), (POLYVINYL ALCOHOL\POVIDONE:Back pain:0.67), (PRAMOXINE HYDROCHLORIDE:Back pain:1.08), (PRASUGREL:Back pain:0.35), (PREDNISOLONE ACETATE\SULFACETAMIDE SODIUM:Back pain:0.4), (PREDNISOLONE SODIUM METAZOATE:Back pain:0.17), (PROBENECID:Back pain:0.36), (PROPATYL NITRATE:Back pain:0.68), (PROTRIPTYLINE HYDROCHLORIDE:Back pain:1.83), (PSORALEN:Back pain:0.08), (PYGEUM:Back pain:0.11), (PYRAZINAMIDE:Back pain:0.8), (PYRIDOSTIGMINE:Back pain:0.44), (PYRIDOXAL PHOSPHATE:Back pain:0.89), (PYRIMETHAMINE:Back pain:0.67), (QUERCETIN:Back pain:0.17), (QUINACRINE:Back pain:1.98), (QUINAGOLIDE HYDROCHLORIDE:Back pain:0.03), (QUINIDINE GLUCONATE:Back pain:0.25), (RASAGILINE:Back pain:0.29), (RESVERATROL:Back pain:0), (RETINOL:Back pain:1.95), (RUCAPARIB CAMSYLATE:Back pain:0.4), (RUFINAMIDE:Back pain:0.33), (SCOPOLAMINE HYDROBROMIDE:Back pain:0.15), (SELENIUM SULFIDE:Back pain:0.37), (SELEXIPAG:Back pain:0.45), (SENNOSIDE B:Back pain:0.09), (SEVOFLURANE:Back pain:0.84), (SILICON DIOXIDE:Back pain:0.79), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Back pain:0.37), (SODIUM PHOSPHATE, MONOBASIC, ANHYDROUS:Back pain:0.03), (SUGAMMADEX:Back pain:0.58), (SULFACETAMIDE SODIUM\SULFUR:Back pain:0.31), (SULTOPRIDE HYDROCHLORIDE:Back pain:0.35), (SUPLATAST TOSILATE:Back pain:1.66), (TAFAMIDIS MEGLUMINE:Back pain:0.45), (TAZAROTENE:Back pain:0.06), (TBO-FILGRASTIM:Back pain:0.46), (TEDUGLUTIDE\WATER:Back pain:1.99), (TEGASEROD MALEATE:Back pain:0.85), (TELOTRISTAT ETHYL:Back pain:1.08), (TEMSIROLIMUS:Back pain:0.81), (TERCONAZOLE:Back pain:0.87), (TETRACAINE HYDROCHLORIDE:Back pain:0.06), (TETRACYCLINE:Back pain:2.26), (THIOCTIC ACID:Back pain:0.96), (THIOPENTAL SODIUM:Back pain:0.15), (THROMBOMODULIN ALFA:Back pain:0.77), (TIAPRIDE:Back pain:0.2), (TIAPROFENIC ACID:Back pain:0.1), (TIGECYCLINE:Back pain:0.76), (TILIDINE HYDROCHLORIDE:Back pain:0.38), (TINIDAZOLE:Back pain:0.4), (TIOPRONIN:Back pain:0.06), (TIQUIZIUM BROMIDE:Back pain:0.66), (TOPIROXOSTAT:Back pain:0.24), (TOREMIFENE CITRATE:Back pain:0.64), (TOSUFLOXACIN TOSYLATE:Back pain:0.74), (TRANILAST:Back pain:0.15), (TREPROSTINIL DIOLAMINE:Back pain:0.28), (TRIMETAZIDINE:Back pain:0.51), (TRIPTORELIN:Back pain:0.02), (TRIPTORELIN PAMOATE:Back pain:1.46), (TRYPTOPHAN:Back pain:0.35), (VECURONIUM BROMIDE:Back pain:0.95), (VELAGLUCERASE ALFA:Back pain:0.34), (VINPOCETINE:Back pain:0.33), (VORAPAXAR SULFATE:Back pain:0.13), (ZANAMIVIR:Back pain:0.16), (ZINC ACETATE:Back pain:0.09), (ZINC CITRATE:Back pain:0.13), (ZOTEPINE:Back pain:0.73), (ZUCLOPENTHIXOL DECANOATE:Back pain:0.74), (ACETAMINOPHEN\CODEINE PHOSPHATE:Bacteraemia:0.31), (AMITRIPTYLINE:Bacteraemia:0.07), (BORTEZOMIB:Bact eraemia:0.32), (CALCIUM:Bacteraemia:0.18), (CANDESARTAN:Bacteraemia:0.13), (CELECO XIB:Bacteraemia:0.24), (CERTOLIZUMAB PEGOL:Bacteraemia:0.77), (CLOZAPINE:Bacteraemia:0.06), (INDAPAMIDE\PERINDOPRIL: Bacteraemia:0.07), (IRON:Bacteraemia:0.35), (LERCANIDIPINE:Bacteraemia:0.16), (NAPROX EN:Bacteraemia:0.58), (NILOTINIB:Bacteraemia:0.12), (NITROGLYCERIN:Bacteraemia:0.04), (PONATINIB:Bacteraemia:0.45), (PRAVASTATIN:Bacteraemia:0.08), (SULFAMETHOXAZO LE:Bacteraemia:0.17), (TELMISARTAN:Bacteraemia:0.01), (TIOTROPIUM BROMIDE MONOHYDRATE:Bacteraemia:0.22), (VERAPAMIL HYDROCHLORIDE:Bacteraemia:0.21), (.ALPHA.1-PROTEINASE INHIBITOR HUMAN:Bacterial infection:0.28), (ACARBOSE:Bacterial infection:0.11), (ACETAMINOPHEN\OXYCODONE\OXYCODONE HYDROCHLORIDE:Bacterial infection:0.19), (ACETAZOLAMIDE:Bacterial infection:0.58), (ADO-TRASTUZUMAB EMTANSINE:Bacterial infection:0), (ALCOHOL:Bacterial infection:0.25), (ALENDRONIC ACID:Bacterial infection:0.48), (AMINOSALICYLIC ACID:Bacterial infection:0.05), (AMISULPRIDE:Bacterial infection:0.37), (ANASTROZOLE:Bacterial infection:1), (ANIDULAFUNGIN:Bacterial infection:0.05),(ARMODAFINIL:Bacterial infection:0.76), (BENAZEPRIL HYDROCHLORIDE:Bacterial infection:0.49), (BENAZEPRIL\BENAZEPRIL HYDROCHLORIDE:Bacterial infection:0.13), (BENDAMUSTINE HYDROCHLORIDE:Bacterial infection:0.37), (BRINZOLAMIDE:Bacterial infection:0.34), (BROTIZOLAM:Bacterial infection:0.21), (CALCITONIN SALMON:Bacterial infection:0.23), (CALCIUM ACETATE:Bacterial infection:0.73), (CALCIUM CARBONATE\VITAMIN D:Bacterial infection:0.41), (CANAKINUMAB:Bacterial infection:0.21), (CAPECITABINE:Bacterial infection:0.06), (CARVEDILOL PHOSPHATE:Bacterial infection:0.57), (CHOLESTYRAMINE:Bacterial infection:1.07), (CICLESONIDE:Bacterial infection:0.27), (CLOBAZAM:Bacterial infection:0.15), (CLOFARABINE:Bacterial infection:0), (CYPROHEPTADINE:Bacterial infection:0.31), (DACLIZUMAB:Bacterial infection:0.55), (DALTEPARIN:Bacterial infection:0.48), (DANAZOL:Bacterial infection:0.28), (DESLORATADINE:Bacterial infection:0.79), (DICLOFENAC:Bacterial infection:1.27), (DIVALPROEX SODIUM:Bacterial infection:1.16), (DOFETILIDE:Bacterial infection:0.3), (DOXAZOSIN\DOXAZOSIN MESYLATE:Bacterial infection:0.43), (ECONAZOLE:Bacterial infection:0.73), (EDOXABAN TOSYLATE:Bacterial infection:0.41), (ELETRIPTAN HYDROBROMIDE:Bacterial infection:0.54), (ELOTUZUMAB:Bacterial infection:0.36), (EPLERENONE:Bacterial infection:0.13), (EVOLOCUMAB:Bacterial infection:0.4), (EZETIMIBE:Bacterial infection:0.82), (EZETIMIBE\SIMVASTATIN:Bacterial infection:0.2), (FERROUS FUMARATE:Bacterial infection:0.11), (FESOTERODINE FUMARATE:Bacterial infection:0.33), (FLUOXETINE\FLUOXETINE HYDROCHLORIDE:Bacterial infection:0.61), (FORMOTEROL FUMARATE\MOMETASONE FUROATE:Bacterial infection:0.2), (GLICLAZIDE:Bacterial infection:0.31), (GLYCOPYRROLATE:Bacterial infection:0.02), (HYDROCHLOROTHIAZIDE\LOSARTAN POTASSIUM:Bacterial infection:0.31), (HYDROCHLOROTHIAZIDE\TELMISARTAN:Bacterial infection:0.05), (HYDROCORTISONE ACETATE:Bacterial infection:0.28), (HYDROXYUREA:Bacterial infection:0.22), (IBRITUMOMAB TIUXETAN:Bacterial infection:0.76), (ILOPROST:Bacterial infection:0.31), (IMIPRAMINE HYDROCHLORIDE:Bacterial infection:0.7), (IRBESARTAN:Bacterial infection:0.77), (IRINOTECAN HYDROCHLORIDE:Bacterial infection:0.36), (IXAZOMIB:Bacterial infection:0.1), (LEDIPASVIR\SOFOSBUVIR:Bacterial infection:0.51), (LEFLUNOMIDE:Bacterial infection:0.11), (LERCANIDIPINE HYDROCHLORIDE:Bacterial infection:0.53), (LEVOLEUCOVORIN CALCIUM:Bacterial infection:0.27), (LIDOCAINE\PRILOCAINE:Bacterial infection:0.36), (METAXALONE:Bacterial infection:0.73), (METHENAMINE HIPPURATE:Bacterial infection:0.22), (METHENOLONE ENANTHATE:Bacterial infection:0.04), (METHOCARBAMOL:Bacterial infection:0.84), (METHYLPHENIDATE HYDROCHLORIDE:Bacterial infection:0.49), (NABUMETONE:Bacterial infection:0.15), (NINTEDANIB:Bacterial infection:0.13), (NORETHINDRONE:Bacterial infection:0.03), (OLODATEROL HYDROCHLORIDE\TIOTROPIUM BROMIDE MONOHYDRATE:Bacterial infection:0.41), (OLOPATADINE:Bacterial infection:0.05), (OMEGA-3-ACID ETHYL ESTERS:Bacterial infection:0.3), (OXAZEPAM:Bacterial infection:0.7), (PAMIDRONATE DISODIUM:Bacterial infection:0.29), (PANTOPRAZOLE MAGNESIUM:Bacterial infection:1.06), (PAROXETINE\PAROXETINE HYDROCHLORIDE:Bacterial infection:0.72), (PAZOPANIB HYDROCHLORIDE:Bacterial infection:0.03), (PENTOSTATIN:Bacterial infection:0.27), (PHENOBARBITAL:Bacterial infection:0.93), (PRAMIPEXOLE DIHYDROCHLORIDE:Bacterial infection:0.68), (PROPRANOLOL\PROPRANOLOL HYDROCHLORIDE:Bacterial infection:0.25), (RISEDRONATE SODIUM:Bacterial infection:0.55), (RIZATRIPTAN BENZOATE:Bacterial infection:0.51), (SACUBITRIL\VALSARTAN:Bacterial infection:0.6), (SCOPOLAMINE:Bacterial infection:0.46), (SECUKINUMAB:Bacterial infection:0.55), (SUMATRIPTAN\SUMATRIPTAN SUCCINATE:Bacterial infection:0.69), (TERBINAFINE HYDROCHLORIDE:Bacterial infection:0.16), (TINZAPARIN:Bacterial infection:0.15), (TINZAPARIN SODIUM:Bacterial infection:0.23), (TRIAMCINOLONE ACETONIDE:Bacterial infection:1.05), (UMECLIDINIUM BROMIDE\VILANTEROL TRIFENATATE:Bacterial infection:0.42), (VARENICLINE TARTRATE:Bacterial infection:1.14), (VITAMIN A:Bacterial infection:0.15), (VITAMIN B:Bacterial infection:0.74), (ACETAMINOPHEN\BUTALBITAL\CAFFEINE:Balance disorder:0.15), (BISOPROLOL FUMARATE:Balance disorder:0.17), (CALCIUM CARBONATE\CHOLECALCIFEROL:Balance disorder:0.36), (CERTOLIZUMAB PEGOL:Balance disorder:0.32), (CORTISONE\HYDROCORTISONE:Balance disorder:0.17), (EPINEPHRINE:Balance disorder:0.11), (EVEROLIMUS:Balance disorder:0.18), (FENOFIBRATE:Balance disorder:0.28), (FERROUS SULFATE:Balance disorder:0.17), (FERROUS SULFATE\FERROUS SULFATE, DRIED:Balance disorder:0.24), (GLIPIZIDE:Balance disorder:0.09), (HYDROMORPHONE HYDROCHLORIDE:Balance disorder:0.33), (IBRUTINIB:Balance disorder:0.39), (INSULIN ASPART:Balance disorder:0.03), (METRONIDAZOLE:Balance disorder:0.76), (NORTRIPTYLINE:Balance disorder:0.18), (OLANZAPINE:Balance disorder:0.4), (OXCARBAZEPINE:Balance disorder:0.68), (PANTOPRAZOLE:Balance disorder:0.97), (POLYETHYLENE GLYCOLS:Balance disorder:0.13), (QUETIAPINE:Balance disorder:0.24), (SACUBITRIL\VALSARTAN:Balance disorder:0.62), (SODIUM OXYBATE:Balance disorder:0.3), (TEMAZEPAM:Balance disorder:0.36), (WARFARIN SODIUM:Balance disorder:0.49), (ACARBOSE:Barrett's oesophagus:0.16), (ACETAMINOPHEN\OXYCODONE\OXYCODONE HYDROCHLORIDE:Barrett's oesophagus:0.08), (ALCOHOL:Barrett's oesophagus:0.01), (AMLODIPINE BESYLATE\HYDROCHLOROTHIAZIDE\OLMESARTAN MEDOXOMIL:Barrett's oesophagus:0.03), (AZELASTINE:Barrett's oesophagus:0.05), (BENAZEPRIL\BENAZEPRIL HYDROCHLORIDE:Barrett's oesophagus:0.1), (BISACODYL:Barrett's oesophagus:0.29), (BISACODYL OR DOCUSATE SODIUM:Barrett's oesophagus:0.87), (BROMAZEPAM:Barrett's oesophagus:0.09), (BUDESONIDE\FORMOTEROL FUMARATE:Barrett's oesophagus:0.26), (BUPRENORPHINE HYDROCHLORIDE:Barrett's oesophagus:0.18), (BUPRENORPHINE HYDROCHLORIDE\NALOXONE HYDROCHLORIDE:Barrett's oesophagus:0.46), (CALCIUM ACETATE:Barrett's oesophagus:0.48), (CALCIUM CARBONATE\VITAMIN D:Barrett's oesophagus:0.14), (CANAGLIFLOZIN\METFORMIN HYDROCHLORIDE:Barrett's oesophagus:0.09), (CAPTOPRIL:Barrett's oesophagus:0.23), (CARFILZOMIB:Barrett's oesophagus:0.31), (CARVEDILOL PHOSPHATE:Barrett's oesophagus:0.72), (CEPHALEXIN:Barrett's oesophagus:0.88), (CETIRIZINE HYDROCHLORIDE\PSEUDOEPHEDRINE HYDROCHLORIDE:Barrett's oesophagus:0.03), (CORTICOTROPIN:Barrett's oesophagus:0.08), (DACLIZUMAB:Barrett's oesophagus:0.38), (DARBEPOETIN ALFA:Barrett's oesophagus:0.16), (DASATINIB:Barrett's oesophagus:1.2), (DESLORATADINE:Barrett's oesophagus:0.49), (DOXEPIN HYDROCHLORIDE:Barrett's oesophagus:0.04), (DRONABINOL:Barrett's oesophagus:0.26), (ELETRIPTAN HYDROBROMIDE:Barrett's oesophagus:0.84), (EMPAGLIFLOZIN\LINAGLIPTIN:Barrett's oesophagus:0.22), (ENALAPRIL MALEATE:Barrett's oesophagus:0.85), (ENOXAPARIN:Barrett's oesophagus:0.95), (ESOMEPRAZOLE MAGNESIUM\NAPROXEN:Barrett's oesophagus:0.32), (ESZOPICLONE:Barrett's oesophagus:0.79), (FEBUXOSTAT:Barrett's oesophagus:0.04), (FINGOLIMOD HYDROCHLORIDE:Barrett's oesophagus:0.01), (FLUTICASONE FUROATE\SALMETEROL XINAFOATE:Barrett's oesophagus:0.01), (FORMOTEROL FUMARATE:Barrett's oesophagus:0.16), (GLYCOPYRRONIUM\INDACATEROL:Barrett's oesophagus:0.09), (HYDROCHLOROTHIAZIDE\IRBESARTAN:Barrett's oesophagus:0.26), (HYDROCHLOROTHIAZIDE\VALSARTAN:Barrett's oesophagus:0.44), (HYDROXYCHLOROQUINE DIPHOSPHATE:Barrett's oesophagus:0.42), (HYDROXYUREA:Barrett's oesophagus:0.09), (HYDROXYZINE PAMOATE:Barrett's oesophagus:0.84), (ILOPROST:Barrett's oesophagus:0.32), (INDAPAMIDE:Barrett's oesophagus:0.37), (INDAPAMIDE\PERINDOPRIL:Barrett's oesophagus:0.05), (INDOMETHACIN:Barrett's oesophagus:0.33), (LERCANIDIPINE HYDROCHLORIDE:Barrett's oesophagus:0.24), (LOMITAPIDE MESYLATE:Barrett's oesophagus:0.27), (METHENAMINE HIPPURATE:Barrett's oesophagus:0.22), (MIDODRINE:Barrett's oesophagus:0.02), (MOXIFLOXACIN HYDROCHLORIDE:Barrett's oesophagus:0.72), (NALOXONE HYDROCHLORIDE\OXYCODONE HYDROCHLORIDE:Barrett's oesophagus:0.53), (NILOTINIB:Barrett's oesophagus:0.41), (ONABOTULINUMTOXINA:Barrett's oesophagus:0.19), (PANTOPRAZOLE MAGNESIUM:Barrett's oesophagus:1.22), (PAROXETINE:Barrett's oesophagus:0.68), (PEGINTERFERON BETA-1A:Barrett's oesophagus:0.39), (PHENOBARBITAL:Barrett's oesophagus:0.59), (PRAZOSIN:Barrett's oesophagus:0.01), (PREDNISOLONE ACETATE:Barrett's oesophagus:0.08), (PROPAFENONE HYDROCHLORIDE:Barrett's oesophagus:0.01), (SACUBITRIL\VALSARTAN:Barrett's oesophagus:0.72), (SCOPOLAMINE:Barrett's oesophagus:0.11), (SECUKINUMAB:Barrett's oesophagus:0.71), (SUMATRIPTAN:Barrett's oesophagus:0.29), (TETRACYCLINE:Barrett's oesophagus:0.52), (TRASTUZUMAB:Barrett's oesophagus:0.04), (UMECLIDINIUM BROMIDE\VILANTEROL TRIFENATATE:Barrett's oesophagus:1.06), (ZONISAMIDE:Barrett's oesophagus:0.25), (ACARBOSE:Basal cell carcinoma:0.16), (ACETAMINOPHEN\BUTALBITAL\CAFFEINE:Basal cell carcinoma:0.55), (ACETAZOLAMIDE:Basal cell carcinoma:0.17), (ALENDRONIC ACID:Basal cell carcinoma:0.18), (ALISKIREN:Basal cell carcinoma:0.01), (AMLODIPINE BESYLATE\HYDROCHLOROTHIAZIDE\OLMESARTAN MEDOXOMIL:Basal cell carcinoma:0.22), (AMPHETAMINE ASPARTATE\AMPHETAMINE SULFATE\DEXTROAMPHETAMINE SACCHARATE\DEXTROAMPHETAMINE SULFATE:Basal cell carcinoma:0.42), (BORTEZOMIB:Basal cell carcinoma:0.26), (BUDESONIDE\FORMOTEROL FUMARATE:Basal cell carcinoma:0.11), (CARFILZOMIB:Basal cell carcinoma:0.14), (CARVEDILOL PHOSPHATE:Basal cell carcinoma:0.62), (CERTOLIZUMAB PEGOL:Basal cell carcinoma:0.3), (CLOZAPINE:Basal cell carcinoma:0.1), (CORTISONE\HYDROCORTISONE:Basal cell carcinoma:0.09), (DABIGATRAN:Basal cell carcinoma:0.29), (DALTEPARIN:Basal cell carcinoma:0.08), (DEVICE:Basal cell carcinoma:0.03), (DIPHENHYDRAMINE:Basal cell carcinoma:0.72), (DOFETILIDE:Basal cell carcinoma:0.52), (ECONAZOLE:Basal cell carcinoma:0.06), (EDOXABAN TOSYLATE:Basal cell carcinoma:0.26), (ELETRIPTAN HYDROBROMIDE:Basal cell carcinoma:0.08), (ENOXAPARIN:Basal cell carcinoma:1.62), (ESZOPICLONE:Basal cell carcinoma:0.46), (EVEROLIMUS:Basal cell carcinoma:0.89), (EVOLOCUMAB:Basal cell carcinoma:0.5), (FEBUXOSTAT:Basal cell carcinoma:0.13), (FESOTERODINE FUMARATE:Basal cell carcinoma:0.67), (FLUDROCORTISONE ACETATE:Basal cell carcinoma:0.1), (GLYCOPYRRONIUM\INDACATEROL:Basal cell carcinoma:0.47), (HYDROCHLOROTHIAZIDE\TELMISARTAN:Basal cell carcinoma:0.09), (HYDROCODONE:Basal cell carcinoma:0.8), (HYDROXYUREA:Basal cell carcinoma:0.02), (HYOSCYAMINE:Basal cell carcinoma:0.27), (ILOPROST:Basal cell carcinoma:0.17), (IMATINIB MESYLATE:Basal cell carcinoma:0.07), (INDAPAMIDE\PERINDOPRIL:Basal cell carcinoma:1), (LITHIUM CARBONATE:Basal cell carcinoma:0.26), (METHYLPREDNISOLONE ACETATE:Basal cell carcinoma:0.1), (METRONIDAZOLE\METRONIDAZOLE HYDROCHLORIDE:Basal cell carcinoma:0.25), (MOXIFLOXACIN HYDROCHLORIDE:Basal cell carcinoma:0.73), (MOXONIDINE:Basal cell carcinoma:0.04), (NADOLOL:Basal cell carcinoma:0.33), (NILOTINIB:Basal cell carcinoma:0.42), (OMEGA-3-ACID ETHYL ESTERS:Basal cell carcinoma:0.92), (PANTOPRAZOLE MAGNESIUM:Basal cell carcinoma:0.76), (PAROXETINE\PAROXETINE HYDROCHLORIDE:Basal cell carcinoma:0.61), (PAZOPANIB HYDROCHLORIDE:Basal cell carcinoma:0.06), (PENICILLIN:Basal cell carcinoma:0.12), (PENTOXIFYLLINE:Basal cell carcinoma:0.01), (PHENOBARBITAL:Basal cell carcinoma:0.61), (PONATINIB:Basal cell carcinoma:0.32), (POTASSIUM:Basal cell carcinoma:1.4), (PREDNISOLONE ACETATE:Basal cell carcinoma:0.01), (PROPAFENONE HYDROCHLORIDE:Basal cell carcinoma:0.19), (PYRIDOXINE:Basal cell carcinoma:0.21), (SACUBITRIL\VALSARTAN:Basal cell carcinoma:0.55), (SITAGLIPTIN:Basal cell carcinoma:0.23), (SODIUM OXYBATE:Basal cell carcinoma:0.2), (SOTALOL:Basal cell carcinoma:1.28), (SUNITINIB MALATE:Basal cell carcinoma:0.1), (TERIFLUNOMIDE:Basal cell carcinoma:0.03), (TETRACYCLINE:Basal cell carcinoma:0.54), (THALIDOMIDE:Basal cell carcinoma:0.02), (THYROID, PORCINE:Basal cell carcinoma:0.13), (TINZAPARIN SODIUM:Basal cell carcinoma:0.07), (TIOTROPIUM:Basal cell carcinoma:0.3), (TIZANIDINE HYDROCHLORIDE:Basal cell carcinoma:0.34), (TOLTERODINE TARTRATE:Basal cell carcinoma:0.01), (UMECLIDINIUM BROMIDE\VILANTEROL TRIFENATATE:Basal cell carcinoma:0.44), (VARENICLINE TARTRATE:Basal cell carcinoma:0.22), (VITAMIN B:Basal cell carcinoma:0.86), (ZINC:Basal cell carcinoma:0.12), (ALCOHOL:Basedow's disease:0.06), (AMIODARONE:Basedow's disease:0.38), (AMIODARONE HYDROCHLORIDE:Basedow's disease:0.3), (BISACODYL OR DOCUSATE SODIUM:Basedow's disease:0.1), (BUMETANIDE:Basedow's disease:0.09), (BUSPIRONE HYDROCHLORIDE:Basedow's disease:0.67), (CANDESARTAN CILEXETIL\HYDROCHLOROTHIAZIDE:Basedow's disease:0.13), (CIPROFLOXACIN:Basedow's disease:0.11), (CLOTRIMAZOLE:Basedow's disease:0.09), (CORTISONE\HYDROCORTISONE:Basedow's disease:0.45), (CYCLOBENZAPRINE HYDROCHLORIDE:Basedow's disease:0.19), (DACLIZUMAB:Basedow's disease:0.1), (DASATINIB:Basedow's disease:0.34), (DICLOFENAC:Basedow's disease:0.34), (DICYCLOMINE HYDROCHLORIDE:Basedow's disease:0.22), (DIMENHYDRINATE:Basedow's disease:0.11), (DIVALPROEX SODIUM:Basedow's disease:0.79), (DOXAZOSIN\DOXAZOSIN MESYLATE:Basedow's disease:0.11), (DULOXETINE:Basedow's disease:0.54), (ENALAPRIL MALEATE:Basedow's disease:0.26), (ENOXAPARIN:Basedow's disease:0.39), (ESOMEPRAZOLE:Basedow's disease:0.41), (EVEROLIMUS:Basedow's disease:0.85), (EVOLOCUMAB:Basedow's disease:1.06), (EZETIMIBE\SIMVASTATIN:Basedow's disease:0.14), (FLUTICASONE:Basedow's disease:0.01), (HYDROCHLOROTHIAZIDE\LISINOPRIL:Basedow's disease:0.49), (IBRUTINIB:Basedow's disease:1.06), (IRON:Basedow's disease:0.85), (LISDEXAMFETAMINE DIMESYLATE:Basedow's disease:0.57), (LITHIUM CARBONATE:Basedow's disease:0.54), (LOSARTAN POTASSIUM:Basedow's disease:0.19), (METAXALONE:Basedow's disease:0.2), (METHYLPREDNISOLONE ACETATE:Basedow's disease:0.14), (MOXIFLOXACIN HYDROCHLORIDE:Basedow's disease:0.14), (NEBIVOLOL:Basedow's disease:0.12), (NITROGLYCERIN:Basedow's disease:0.11), (NORTRIPTYLINE:Basedow's disease:0.11), (OMEGA-3-ACID ETHYL ESTERS:Basedow's disease:0.12), (PAROXETINE:Basedow's disease:0.01), (PAROXETINE\PAROXETINE HYDROCHLORIDE:Basedow's disease:0.24), (PAZOPANIB HYDROCHLORIDE:Basedow's disease:0), (PEGINTERFERON BETA-1A:Basedow's disease:0.11), (PHENYTOIN:Basedow's disease:0.06), (PRAMIPEXOLE:Basedow's disease:0.03), (PYRAZINAMIDE:Basedow's disease:0.67), (RALOXIFENE HYDROCHLORIDE:Basedow's disease:0.24), (RANOLAZINE:Basedow's disease:0.41), (RISPERIDONE:Basedow's disease:0.1), (SACUBITRIL\VALSARTAN:Basedow's disease:0.78), (SUMATRIPTAN:Basedow's disease:0.3), (TAMSULOSIN HYDROCHLORIDE:Basedow's disease:0.25), (THALIDOMIDE:Basedow's disease:0.01), (TOLTERODINE TARTRATE:Basedow's disease:0.01), (UMECLIDINIUM BROMIDE\VILANTEROL TRIFENATATE:Basedow's disease:0.04), (BUMETANIDE:Behcet's syndrome:0.15), (CEFTRIAXONE SODIUM:Behcet's syndrome:0.13), (CLINDAMYCIN\CLINDAMYCIN PHOSPHATE:Behcet's syndrome:0.35), (DASATINIB:Behcet's syndrome:0.04), (DIGOXIN:Behcet's syndrome:0.23), (DILTIAZEM:Behcet's syndrome:0.23), (DIVALPROEX SODIUM:Behcet's syndrome:0.54), (DOCUSATE SODIUM:Behcet's syndrome:0.54), (DULOXETINE:Behcet's syndrome:0.19), (ESZOPICLONE:Behcet's syndrome:0.56), (EVEROLIMUS:Behcet's syndrome:0.13), (EVOLOCUMAB:Behcet's syndrome:0.65), (GLIPIZIDE:Behcet's syndrome:0.03), (HYDROCHLOROTHIAZIDE\LISINOPRIL:Behcet's syndrome:0.24), (HYDROXYCHLOROQUINE DIPHOSPHATE:Behcet's syndrome:0.29), (IBRUTINIB:Behcet's syndrome:0.77), (IMATINIB MESYLATE:Behcet's syndrome:0.1), (LOVASTATIN:Behcet's syndrome:0.7), (METHYLPREDNISOLONE ACETATE:Behcet's syndrome:0.21), (METRONIDAZOLE\METRONIDAZOLE HYDROCHLORIDE:Behcet's syndrome:0.32), (NEBIVOLOL:Behcet's syndrome:0.38), (NILOTINIB:Behcet's syndrome:0.05), (PROBIOTICS NOS:Behcet's syndrome:0.07), (SECUKINUMAB:Behcet's syndrome:0.48), (SULFAMETHOXAZOLE:Behcet's syndrome:0), (PYRAZINAMIDE:Benign neoplasm of bladder:0.52), (.ALPHA.1-PROTEINASE INHIBITOR HUMAN:Benign prostatic hyperplasia:0.65), (ACETAMINOPHEN\APRONALIDE\CAFFEINE\PROPYPHENAZONE:Be nign prostatic hyperplasia:0.41), (ACETAMINOPHEN\CAFFEINE\PROMETHAZINE\SALICYLAMIDE:Ben ign prostatic hyperplasia:0.38), (ACETAMINOPHEN\PROPOXYPHENE HYDROCHLORIDE:Benign prostatic hyperplasia:0.79), (ACETAMINOPHEN\PROPOXYPHENE NAPSYLATE:Benign prostatic hyperplasia:0.06), (ACITRETIN:Benign prostatic hyperplasia:1.08), (ADENINE:Benign prostatic hyperplasia:1.43), (AGALSIDASE BETA:Benign prostatic hyperplasia:1.22), (ALISKIREN HEMIFUMARATE\HYDROCHLOROTHIAZIDE:Benign prostatic hyperplasia:0.02), (ALUMINUM HYDROXIDE\MAGNESIUM OXIDE\OXETHAZAINE:Benign prostatic hyperplasia:0.16), (AMINOSALICYLIC ACID:Benign prostatic hyperplasia:0.31), (AMMONIUM CHLORIDE\DEXTROMETHORPHAN\SODIUM CITRATE:Benign prostatic hyperplasia:0.11), (AMOXAPINE:Benign prostatic hyperplasia:1.48), (AMPICILLIN SODIUM:Benign prostatic hyperplasia:0.29), (ANAGRELIDE:Benign prostatic hyperplasia:0.08), (ARIPIPRAZOLE LAUROXIL:Benign prostatic hyperplasia:0.25), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Benign prostatic hyperplasia:0.36), (ASPIRIN\CLOPIDOGREL BISULFATE:Benign prostatic hyperplasia:0.21), (ASPIRIN\DIHYDROXYALUMINUM AMINOACETATE ANHYDROUS\MAGNESIUM CARBONATE:Benign prostatic hyperplasia:1.03), (ASPIRIN\DIHYDROXYALUMINUM AMINOACETATE\MAGNESIUM CARBONATE:Benign prostatic hyperplasia:0.16), (ATAZANAVIR:Benign prostatic hyperplasia:0.2), (ATORVASTATIN CALCIUM\EZETIMIBE:Benign prostatic hyperplasia:0.29), (AVOCADO OIL\SOYBEAN OIL:Benign prostatic hyperplasia:0.03), (AZELASTINE HYDROCHLORIDE\FLUTICASONE PROPIONATE:Benign prostatic hyperplasia:0.33), (AZITHROMYCIN OR METHOTREXATE SODIUM:Benign prostatic hyperplasia:0.12), (BALSALAZIDE DISODIUM:Benign prostatic hyperplasia:0.16), (BECLOMETHASONE DIPROPIONATE\FORMOTEROL:Benign prostatic hyperplasia:0.47), (BENSERAZIDE\LEVODOPA:Benign prostatic hyperplasia:0.93), (BENZTROPINE:Benign prostatic hyperplasia:0.67), (BENZTROPINE MESYLATE:Benign prostatic hyperplasia:0.6), (BETAMETHASONE DIPROPIONATE\CLOTRIMAZOLE:Benign prostatic hyperplasia:0.99), (BETAMETHASONE SODIUM PHOSPHATE\NEOMYCIN SULFATE:Benign prostatic hyperplasia:0.49), (BETAMETHASONE\CALCIPOTRIENE:Benign prostatic hyperplasia:0.93), (BILASTINE:Benign prostatic hyperplasia:1.16), (BIPERIDEN:Benign prostatic hyperplasia:0.34), (BIXALOMER:Benign prostatic hyperplasia:0.27), (BLINATUMOMAB:Benign prostatic hyperplasia:0.81), (BROMPERIDOL:Benign prostatic hyperplasia:0.09), (BUCILLAMINE:Benign prostatic hyperplasia:1.54), (BUFORMIN:Benign prostatic hyperplasia:0.26), (CABOZANTINIB:Benign prostatic hyperplasia:0.18), (CALCITONIN:Benign prostatic hyperplasia:1.27), (CALCITONIN SALMON:Benign prostatic hyperplasia:1.2), (CALCIUM CARBONATE\ERGOCALCIFEROL\RETINOL:Benign prostatic hyperplasia:0.94), (CALCIUM CITRATE\VITAMIN D:Benign prostatic hyperplasia:0.09), (CALCIUM PANTOTHENATE\CYANOCOBALAMIN\NIACINAMIDE\PYRIDOXINE HYDROCHLORIDE\RIBOFLAVIN\THIAMINE MONONITRATE:Benign prostatic hyperplasia:0.21), (CALCIUM\MAGNESIUM:Benign prostatic hyperplasia:1.04), (CAMPHOR\MENTHOL\METHYL SALICYLATE:Benign prostatic hyperplasia:0.38), (CAPSAICIN:Benign prostatic hyperplasia:0.41), (CARBOHYDRATES\FATTY ACIDS\MINERALS\PROTEIN\VITAMINS:Benign prostatic hyperplasia:0.17), (CARTEOLOL:Benign prostatic hyperplasia:0.25), (CASPOFUNGIN:Benign prostatic hyperplasia:0.46), (CEFEPIME\CEFEPIME HYDROCHLORIDE:Benign prostatic hyperplasia:0.59), (CEFOTAXIME SODIUM:Benign prostatic hyperplasia:1.7), (CEFOTAXIME\CEFOTAXIME SODIUM:Benign prostatic hyperplasia:0.17), (CERTOPARIN SODIUM:Benign prostatic hyperplasia:0.63), (CEVIMELINE HYDROCHLORIDE:Benign prostatic hyperplasia:0.44), (CHLOROTHIAZIDE:Benign prostatic hyperplasia:0.22), (CIPROFIBRATE:Benign prostatic hyperplasia:1.41), (CLENBUTEROL HYDROCHLORIDE:Benign prostatic hyperplasia:0.94), (CLIKSTAR:Benign prostatic hyperplasia:0.51), (CLOXACILLIN SODIUM:Benign prostatic hyperplasia:0.16), (COBICISTAT\ELVITEGRAVIR\EMTRICITABINE\TENOFOVIR ALAFENAMIDE FUMARATE:Benign prostatic hyperplasia:0.14), (COLESTILAN CHLORIDE:Benign prostatic hyperplasia:0.1), (COLISTIN:Benign prostatic hyperplasia:0.06), (CORTICOTROPIN:Benign prostatic hyperplasia:1.09), (CORTISONE ACETATE:Benign prostatic hyperplasia:0.06), (CYAMEMAZINE:Benign prostatic hyperplasia:0.84), (CYANAMIDE:Benign prostatic hyperplasia:0.05), (CYANOCOBALAMIN\LIDOCAINE HYDROCHLORIDE\PYRIDOXINE HYDROCHLORIDE\THIAMINE HYDROCHLORIDE:Benign prostatic hyperplasia:0.12), (CYPROHEPTADINE:Benign prostatic hyperplasia:0.91), (CYPROHEPTADINE HYDROCHLORIDE:Benign prostatic hyperplasia:0.39), (DACLIZUMAB:Benign prostatic hyperplasia:1.53), (DEFLAZACORT:Benign prostatic hyperplasia:2.17), (DEXMEDETOMIDINE HYDROCHLORIDE:Benign prostatic hyperplasia:0.13), (DEXTROAMPHETAMINE SULFATE:Benign prostatic hyperplasia:0.72), (DIMEMORFAN PHOSPHATE:Benign prostatic hyperplasia:0.29), (DIMETHICONE\SILICON DIOXIDE\SIMETHICONE:Benign prostatic hyperplasia:0.05), (DIOSMIN:Benign prostatic hyperplasia:0.32), (DOLASETRON MESYLATE:Benign prostatic hyperplasia:0.6), (EDARAVONE:Benign prostatic hyperplasia:0.97), (ELBASVIR:Benign prostatic hyperplasia:0.39), (ELCATONIN:Benign prostatic hyperplasia:0.26), (EMPAGLIFLOZIN\LINAGLIPTIN:Benign prostatic hyperplasia:1.27), (ERIBULIN:Benign prostatic hyperplasia:0.92), (ERIBULIN MESYLATE:Benign prostatic hyperplasia:0.43), (ERYTHROMYCIN\SULFISOXAZOLE:Benign prostatic hyperplasia:0.36), (ESTRIOL:Benign prostatic hyperplasia:0.25), (ESTROGENS, CONJUGATED\MEDROXYPROGESTERONE ACETATE:Benign prostatic hyperplasia:0.78), (ESTRONE\SUS SCROFA OVARY:Benign prostatic hyperplasia:0.38), (ETHACRYNIC ACID:Benign prostatic hyperplasia:0.28), (ETOPOSIDE PHOSPHATE:Benign prostatic hyperplasia:0.04), (ETRETINATE:Benign prostatic hyperplasia:2.56), (EXEMESTANE:Benign prostatic hyperplasia:1.44), (FAROPENEM SODIUM:Benign prostatic hyperplasia:0.58), (FELBINAC:Benign prostatic hyperplasia:0.33), (FERRIC CARBOXYMALTOSE:Benign prostatic hyperplasia:0.11), (FERRIC OXYHYDROXIDE:Benign prostatic hyperplasia:0.67), (FLUNARIZINE HYDROCHLORIDE:Benign prostatic hyperplasia:0.23), (FLUOCINOLONE ACETONIDE:Benign prostatic hyperplasia:0.66), (FLURBIPROFEN AXETIL:Benign prostatic hyperplasia:0.08), (FOSFOMYCIN CALCIUM:Benign prostatic hyperplasia:0.24), (FOSPHENYTOIN\FOSPHENYTOIN SODIUM:Benign prostatic hyperplasia:0.3), (FULVESTRANT:Benign prostatic hyperplasia:0.6), (FURSULTIAMINE:Benign prostatic hyperplasia:1.19), (GLIMEPIRIDE\ROSIGLITAZONE MALEATE:Benign prostatic hyperplasia:0.41), (GLYCOPYRROLATE\INDACATEROL MALEATE:Benign prostatic hyperplasia:1.01), (GRAZOPREVIR:Benign prostatic hyperplasia:0.36), (GUANABENZ ACETATE:Benign prostatic hyperplasia:0.89), (GUANFACINE:Benign prostatic hyperplasia:0.89), (GUANFACINE HYDROCHLORIDE:Benign prostatic hyperplasia:0.43), (HYDROCHLOROTHIAZIDE\METOPROLOL SUCCINATE:Benign prostatic hyperplasia:0.74), (IBRITUMOMAB TIUXETAN:Benign prostatic hyperplasia:0.4), (IDARUCIZUMAB:Benign prostatic hyperplasia:0.89), (IFENPRODIL TARTRATE:Benign prostatic hyperplasia:0.2), (IGURATIMOD:Benign prostatic hyperplasia:0.92), (ILOPROST:Benign prostatic hyperplasia:1.55), (IMIPENEM:Benign prostatic hyperplasia:0.32), (IMMUNE GLOBULIN NOS:Benign prostatic hyperplasia:0.23), (INOTUZUMAB OZOGAMICIN:Benign prostatic hyperplasia:0.25), (INSULIN ASPART\INSULIN DEGLUDEC:Benign prostatic hyperplasia:0.7), (INSULIN PORK\INSULIN PURIFIED PORK:Benign prostatic hyperplasia:0.25), (INTERFERON:Benign prostatic hyperplasia:1.55), (INTERFERON ALFA-2A OR INTERFERON ALFA-2B:Benign prostatic hyperplasia:0.08), (INTERFERON ALFA-2B:Benign prostatic hyperplasia:1.54), (INTERFERON ALFA-N1:Benign prostatic hyperplasia:0.03), (IODINE:Benign prostatic hyperplasia:0.94), (ISONIAZID\PYRAZINAMIDE\RIFAMPIN:Benign prostatic hyperplasia:0.31), (ISONIAZID\RIFAMPIN:Benign prostatic hyperplasia:0.51), (ISTRADEFYLLINE:Benign prostatic hyperplasia:0.23), (IVABRADINE HYDROCHLORIDE:Benign prostatic hyperplasia:0.54), (KALLIDINOGENASE:Benign prostatic hyperplasia:0.5), (LABETALOL HYDROCHLORIDE:Benign prostatic hyperplasia:1.59), (LACTASE:Benign prostatic hyperplasia:0.87), (LACTOBACILLUS ACIDOPHILUS\LACTOBACILLUS DELBRUECKII SUBSP. BULGARICUS:Benign prostatic hyperplasia:0.15), (LANDIOLOL HYDROCHLORIDE:Benign prostatic hyperplasia:0.31), (LANREOTIDE:Benign prostatic hyperplasia:0.7), (LAPATINIB DITOSYLATE:Benign prostatic hyperplasia:1.08), (LETROZOLE:Benign prostatic hyperplasia:1.28), (LEVOSIMENDAN:Benign prostatic hyperplasia:0.12), (LINSIDOMINE:Benign prostatic hyperplasia:0.83), (LOBUCAVIR:Benign prostatic hyperplasia:0.09), (LOMUSTINE:Benign prostatic hyperplasia:0.14), (LURASIDONE HYDROCHLORIDE:Benign prostatic hyperplasia:0.53), (LYSOZYME HYDROCHLORIDE:Benign prostatic hyperplasia:0.57), (MAGNESIUM PIDOLATE:Benign prostatic hyperplasia:0.26), (MECLIZINE\MECLIZINE HYDROCHLORIDE:Benign prostatic hyperplasia:1.48), (MEDROXYPROGESTERONE:Benign prostatic hyperplasia:0.12), (MENATETRENONE:Benign prostatic hyperplasia:0.77), (MEPENZOLATE BROMIDE:Benign prostatic hyperplasia:0.39), (MEPOLIZUMAB:Benign prostatic hyperplasia:1.08), (METFORMIN HYDROCHLORIDE\ROSIGLITAZONE MALEATE:Benign prostatic hyperplasia:0.72), (METHENAMINE HIPPURATE:Benign prostatic hyperplasia:1.53), (METRELEPTIN:Benign prostatic hyperplasia:0.15), (MIGLUSTAT:Benign prostatic hyperplasia:0.07), (MIRIPLATIN:Benign prostatic hyperplasia:0.17), (MITIGLINIDE CALCIUM HYDRATE\VOGLIBOSE:Benign prostatic hyperplasia:1.03), (MITOXANTRONE HYDROCHLORIDE:Benign prostatic hyperplasia:0), (NAFAMOSTAT MESYLATE:Benign prostatic hyperplasia:0.14), (NALDEMEDINE:Benign prostatic hyperplasia:0.05), (NARATRIPTAN HYDROCHLORIDE:Benign prostatic hyperplasia:0.3), (NEOMYCIN:Benign prostatic hyperplasia:0.21), (NIRAPARIB:Benign prostatic hyperplasia:1.24), (NOREPINEPHRINE BITARTRATE:Benign prostatic hyperplasia:0.61), (OMEPRAZOLE\SODIUM BICARBONATE:Benign prostatic hyperplasia:0.05), (OPIPRAMOL:Benign prostatic hyperplasia:0.46), (ORPHENADRINE:Benign prostatic hyperplasia:0.76), (OXYBUTYNIN\OXYBUTYNIN CHLORIDE:Benign prostatic hyperplasia:0.57), (PARICALCITOL:Benign prostatic hyperplasia:1.15), (PASIREOTIDE:Benign prostatic hyperplasia:0.65), (PEGINTERFERON ALFA-2A OR PEGINTERFERON ALFA-2B:Benign prostatic hyperplasia:0.1), (PEGINTERFERON BETA-1A:Benign prostatic hyperplasia:1.39), (PEGVISOMANT:Benign prostatic hyperplasia:0.09), (PENICILLIN V POTASSIUM:Benign prostatic hyperplasia:0.12), (PERINDOPRIL ARGININE:Benign prostatic hyperplasia:0.14), (PERPHENAZINE:Benign prostatic hyperplasia:0.18), (PHENOBARBITAL SODIUM:Benign prostatic hyperplasia:0.04), (PHLOROGLUCINOL1,3,5-TRIMETHOXYBENZENE:Benign prostatic hyperplasia:0.85), (PILOCARPINE:Benign prostatic hyperplasia:1.04), (PIRENZEPINE:Benign prostatic hyperplasia:0.04), (POLYSTYRENE SULFONIC ACID:Benign prostatic hyperplasia:0.63), (POTASSIUM CHLORIDE\SODIUM CHLORIDE\SODIUM LACTATE:Benign prostatic hyperplasia:0.23), (POTASSIUM IODIDE:Benign prostatic hyperplasia:0.67), (POTASSIUM PHOSPHATE, MONOBASIC:Benign prostatic hyperplasia:0.02), (PRISTINAMYCIN:Benign prostatic hyperplasia:1.16), (PROMAZINE:Benign prostatic hyperplasia:0.04), (PROMAZINE HYDROCHLORIDE:Benign prostatic hyperplasia:0.09), (PROTRIPTYLINE HYDROCHLORIDE:Benign prostatic hyperplasia:0.07), (PRUCALOPRIDE SUCCINATE:Benign prostatic hyperplasia:0.06), (QUINACRINE:Benign prostatic hyperplasia:0.19), (RALOXIFENE HYDROCHLORIDE:Benign prostatic hyperplasia:1.34), (RALTEGRAVIR:Benign prostatic hyperplasia:0.46), (RETINOL:Benign prostatic hyperplasia:0.87), (RIFABUTIN:Benign prostatic hyperplasia:0.3), (RISEDRONIC ACID:Benign prostatic hyperplasia:0.12), (RIZATRIPTAN:Benign prostatic hyperplasia:0.2), (RUCAPARIB CAMSYLATE:Benign prostatic hyperplasia:0.17), (SARPOGRELATE HYDROCHLORIDE:Benign prostatic hyperplasia:0.95), (SCOPOLAMINE HYDROBROMIDE:Benign prostatic hyperplasia:0.17), (SENNOSIDE B:Benign prostatic hyperplasia:0.05), (SERRAPEPTASE:Benign prostatic hyperplasia:0.11), (SITAFLOXACIN:Benign prostatic hyperplasia:0.46), (SODIUM CITRATE:Benign prostatic hyperplasia:0.16), (SODIUM PHOSPHATE, DIBASIC\SODIUM PHOSPHATE, MONOBASIC:Benign prostatic hyperplasia:0.63), (SOFALCONE:Benign prostatic hyperplasia:0.08), (STREPTOMYCIN\STREPTOMYCIN SULFATE:Benign prostatic hyperplasia:0.17), (SUNITINIB:Benign prostatic hyperplasia:0.05), (SUPLATAST TOSILATE:Benign prostatic hyperplasia:1.14), (TAMOXIFEN CITRATE:Benign prostatic hyperplasia:0.28), (TEDUGLUTIDE\WATER:Benign prostatic hyperplasia:0.51), (TELOTRISTAT ETHYL:Benign prostatic hyperplasia:0.77), (THEOPHYLLINE:Benign prostatic hyperplasia:0.18), (THROMBOMODULIN ALFA:Benign prostatic hyperplasia:0.19), (TIGECYCLINE:Benign prostatic hyperplasia:0.59), (TIMOLOL MALEATE\TRAVOPROST:Benign prostatic hyperplasia:0.87), (TIMOLOL\TRAVOPROST:Benign prostatic hyperplasia:0.77), (TOLCAPONE:Benign prostatic hyperplasia:0.07), (TOSUFLOXACIN TOSYLATE:Benign prostatic hyperplasia:0.21), (TRIMIPRAMINE MALEATE:Benign prostatic hyperplasia:1.18), (TROXIPIDE:Benign prostatic hyperplasia:0.6), (UMECLIDINIUM BROMIDE:Benign prostatic hyperplasia:1.1), (VANDETANIB:Benign prostatic hyperplasia:0.13), (VIDARABINE:Benign prostatic hyperplasia:0.01), (VINORELBINE\VINORELBINE TARTRATE:Benign prostatic hyperplasia:0.64), (ZALTOPROFEN:Benign prostatic hyperplasia:0.47), (ZILEUTON:Benign prostatic hyperplasia:0.72), (ZIPRASIDONE HYDROCHLORIDE\ZIPRASIDONE MESYLATE:Benign prostatic hyperplasia:0.17), (ZIV-AFLIBERCEPT:Benign prostatic hyperplasia:0.14), (ZOLMITRIPTAN:Benign prostatic hyperplasia:0.67), (ZOTEPINE:Benign prostatic hyperplasia:0.28), (ACETAMINOPHEN\CODEINE PHOSPHATE:Biliary cirrhosis primary:0.44), (AMIODARONE:Biliary cirrhosis primary:0.12), (ANASTROZOLE:Biliary cirrhosis primary:0.26), (BISACODYL OR DOCUSATE SODIUM:Biliary cirrhosis primary:0.05), (BISOPROLOL FUMARATE:Biliary cirrhosis primary:0.21), (CYCLOBENZAPRINE HYDROCHLORIDE:Biliary cirrhosis primary:0.03), (DALFAMPRIDINE:Biliary cirrhosis primary:0.2), (DARBEPOETIN ALFA:Biliary cirrhosis primary:0), (DICLOFENAC SODIUM:Biliary cirrhosis primary:0.21), (EVEROLIMUS:Biliary cirrhosis primary:0.47), (FERROUS SULFATE\FERROUS SULFATE, DRIED:Biliary cirrhosis primary:0.47), (IBRUTINIB:Biliary cirrhosis primary:0.44), (ILOPROST:Biliary cirrhosis primary:0.34), (LIDOCAINE:Biliary cirrhosis primary:0.17), (LISDEXAMFETAMINE DIMESYLATE:Biliary cirrhosis primary:0.1), (METOLAZONE:Biliary cirrhosis primary:0.03), (METOPROLOL SUCCINATE:Biliary cirrhosis primary:0.02), (METRONIDAZOLE:Biliary cirrhosis primary:0.73), (METRONIDAZOLE\METRONIDAZOLE HYDROCHLORIDE:Biliary cirrhosis primary:0.11), (MORPHINE:Biliary cirrhosis primary:0.05), (NEBIVOLOL:Biliary cirrhosis primary:0.2), (OMEGA-3-ACID ETHYL ESTERS:Biliary cirrhosis primary:0.01), (PANTOPRAZOLE MAGNESIUM:Biliary cirrhosis primary:0.05), (PRAMIPEXOLE DIHYDROCHLORIDE:Biliary cirrhosis primary:0.28), (RANOLAZINE:Biliary cirrhosis primary:0.5), (SODIUM CHLORIDE:Biliary cirrhosis primary:0.14), (SODIUM OXYBATE:Biliary cirrhosis primary:0.38), (TAMSULOSIN HYDROCHLORIDE:Biliary cirrhosis primary:0.15), (TERIFLUNOMIDE:Biliary cirrhosis primary:0.52), (TIZANIDINE HYDROCHLORIDE:Biliary cirrhosis primary:0.04), (VARENICLINE TARTRATE:Biliary cirrhosis primary:0.13), (VITAMIN B:Biliary cirrhosis primary:0.01), (ABOBOTULINUMTOXINA:Bipolar disorder:0.38), (ACETAMINOPHEN\CAFFEINE\OPIUM:Bipolar disorder:0.8), (ACETAMINOPHEN\PROPOXYPHENE NAPSYLATE:Bipolar disorder:0.62), (ADENOSINE:Bipolar disorder:0), (AGALSIDASE BETA:Bipolar disorder:0.77), (ALUMINUM HYDROXIDE\DIMETHICONE\MAGNESIUM HYDROXIDE:Bipolar disorder:1.27), (AMLODIPINE BESYLATE\TELMISARTAN:Bipolar disorder:0.23), (AMLODIPINE\VALSARTAN:Bipolar disorder:1.08), (AMMONIUM CHLORIDE\DEXTROMETHORPHAN\SODIUM CITRATE:Bipolar disorder:0.35), (AMOBARBITAL SODIUM:Bipolar disorder:0.4), (AMOXICILLIN\CLARITHROMYCIN\ESOMEPRAZOLE:Bipolar disorder:0.06), (AMPHETAMINE\DEXTROAMPHETAMINE:Bipolar disorder:0.37), (ANHYDROUS CITRIC ACID\ASPIRIN\SODIUM BICARBONATE:Bipolar disorder:0.12), (ANTIHISTAMINES NOS:Bipolar disorder:0.76), (ASUNAPREVIR:Bipolar disorder:0.41), (ATROPINE\DIPHENOXYLATE:Bipolar disorder:0.19), (AXITINIB:Bipolar disorder:0.57), (BETAMETHASONE DIPROPIONATE\CLOTRIMAZOLE:Bipolar disorder:0.29), (BETAMETHASONE\CALCIPOTRIENE:Bipolar disorder:0.6), (BILASTINE:Bipolar disorder:0.25), (BLINATUMOMAB:Bipolar disorder:0.08), (BRIVARACETAM:Bipolar disorder:0.56), (BUDESONIDE\FORMOTEROL:Bipolar disorder:0.47), (BUPIVACAINE HYDROCHLORIDE:Bipolar disorder:0.33), (BUTYLSCOPOLAMINE BROMIDE:Bipolar disorder:1.55), (CALCITONIN:Bipolar disorder:0.26), (CALCIUM CARBONATE\ERGOCALCIFEROL\RETINOL:Bipolar disorder:0.55), (CALCIUM\MAGNESIUM:Bipolar disorder:0.97), (CARVEDILOL PHOSPHATE:Bipolar disorder:2.24), (CEFPODOXIME PROXETIL:Bipolar disorder:0.11), (CEVIMELINE HYDROCHLORIDE:Bipolar disorder:0.43), (CHLORAMBUCIL:Bipolar disorder:0.03), (CHOLESTEROL:Bipolar disorder:0.13), (CLIKSTAR:Bipolar disorder:0.09), (COBICISTAT\ELVITEGRAVIR\EMTRICITABINE\TENOFOVIR ALAFENAMIDE FUMARATE:Bipolar disorder:0.37), (COD LIVER OIL:Bipolar disorder:1.05), (COLISTIMETHATE SODIUM:Bipolar disorder:0.25), (CRANBERRY JUICE:Bipolar disorder:0), (CYPROTERONE ACETATE\ETHINYL ESTRADIOL:Bipolar disorder:0.07), (CYTARABINE:Bipolar disorder:0.21), (DABIGATRAN:Bipolar disorder:1.05), (DASATINIB:Bipolar disorder:2.25), (DEFLAZACORT:Bipolar disorder:1.75), (DESFLURANE:Bipolar disorder:0.72), (DEUTETRABENAZINE:Bipolar disorder:0.21), (DEXAMETHASONE ACETATE:Bipolar disorder:0.26), (DEXAMETHASONE SODIUM PHOSPHATE:Bipolar disorder:0.04), (DEXMEDETOMIDINE HYDROCHLORIDE:Bipolar disorder:0.2), (DIAMORPHINE:Bipolar disorder:0.81), (DICLOXACILLIN:Bipolar disorder:0.17), (DIGITOXIN:Bipolar disorder:0.1), (DIHYDROERGOTAMINE MESYLATE:Bipolar disorder:0.05), (DIPYRONE\PROMETHAZINE HYDROCHLORIDE:Bipolar disorder:0.26), (DOFETILIDE:Bipolar disorder:1.43), (DORZOLAMIDE HYDROCHLORIDE:Bipolar disorder:0.18), (ELDECALCITOL:Bipolar disorder:0.14), (ELOTUZUMAB:Bipolar disorder:0.22), (ELTROMBOPAG OLAMINE:Bipolar disorder:0.51), (ESMOLOL HYDROCHLORIDE:Bipolar disorder:0.36), (ESOMEPRAZOLE MAGNESIUM\NAPROXEN:Bipolar disorder:0.64), (ESTRADIOL BENZOATE:Bipolar disorder:0.34), (ESTRADIOL HEMIHYDRATE:Bipolar disorder:0.15), (ESTRADIOL\LEVONORGESTREL:Bipolar disorder:0.18), (ESTRIOL:Bipolar disorder:0.16), (ETHINYL ESTRADIOL\ETHYNODIOL DIACETATE:Bipolar disorder:0.04), (ETHINYL ESTRADIOL\FERROUS FUMARATE\NORETHINDRONE ACETATE:Bipolar disorder:0.33), (FELBAMATE:Bipolar disorder:0.16), (FENOPROFEN CALCIUM:Bipolar disorder:0.07), (FENOTEROL HYDROBROMIDE:Bipolar disorder:0.21), (FERROUS SULFATE\GLYCINE:Bipolar disorder:0.24), (FONDAPARINUX SODIUM:Bipolar disorder:0.29), (FORMOTEROL:Bipolar disorder:1.32), (FORMOTEROL FUMARATE DIHYDRATE:Bipolar disorder:0.06), (FORMOTEROL FUMARATE\MOMETASONE FUROATE:Bipolar disorder:1.11), (FOSFOMYCIN:Bipolar disorder:0.1), (FOSINOPRIL:Bipolar disorder:0.07), (FOSPHENYTOIN\FOSPHENYTOIN SODIUM:Bipolar disorder:0.71), (FUSIDIC ACID:Bipolar disorder:1.34), (GINGER:Bipolar disorder:0.46), (GLIPIZIDE\METFORMIN HYDROCHLORIDE:Bipolar disorder:0.33), (GLYCERIN:Bipolar disorder:0.52), (HERBALS\SAW PALMETTO:Bipolar disorder:0.74), (HYPERICUM PERFORATUM:Bipolar disorder:0.84), (IDELALISIB:Bipolar disorder:0.03), (ILOPROST:Bipolar disorder:0.95), (IMATINIB MESYLATE:Bipolar disorder:1.47), (IMIGLUCERASE:Bipolar disorder:0.26), (IMIQUIMOD:Bipolar disorder:0.17), (INDACATEROL:Bipolar disorder:0.73), (INTERFERON:Bipolar disorder:1.11), (INTERFERON ALFA-2A:Bipolar disorder:0.47), (INTERFERON ALFA-2B:Bipolar disorder:0.43), (INTERFERON GAMMA-1B:Bipolar disorder:0.17), (IRON SUCROSE:Bipolar disorder:0.62), (ISOFLURANE:Bipolar disorder:0.2), (ISOPROPYL ALCOHOL\LEUPROLIDE ACETATE\NORETHINDRONE ACETATE:Bipolar disorder:0.71), (ITRACONAZOLE:Bipolar disorder:0.75), (IVABRADINE HYDROCHLORIDE:Bipolar disorder:0.29), (IVERMECTIN:Bipolar disorder:0.07), (IXAZOMIB:Bipolar disorder:0.34), (LABETALOL HYDROCHLORIDE:Bipolar disorder:1.44), (LANREOTIDE:Bipolar disorder:0.19), (LAPATINIB DITOSYLATE:Bipolar disorder:0.02), (LEUCOVORIN CALCIUM:Bipolar disorder:0.03), (LIDOCAINE\PRILOCAINE:Bipolar disorder:1.03), (LISDEXAMFETAMINE:Bipolar disorder:0.27), (LUMACAFTOR:Bipolar disorder:0.05), (LUTETIUM LU-177:Bipolar disorder:0.05), (LYSERGIDE:Bipolar disorder:0.46), (MAGNESIUM ASPARTATE HYDROCHLORIDE:Bipolar disorder:0.05), (MATRICARIA RECUTITA:Bipolar disorder:0.04), (MEPOLIZUMAB:Bipolar disorder:0.99), (METHSUXIMIDE:Bipolar disorder:0.19), (METHYLPREDNISOLONE ACETATE:Bipolar disorder:1.84), (MIGLUSTAT:Bipolar disorder:0.69), (MOEXIPRIL HYDROCHLORIDE:Bipolar disorder:0.77), (MORPHINE SULFATE\NALTREXONE HYDROCHLORIDE:Bipolar disorder:0.55), (MORPHINE\MORPHINE HYDROCHLORIDE\MORPHINE SULFATE\MORPHINE TARTRATE:Bipolar disorder:0.91), (NALOXONE HYDROCHLORIDE\TILIDINE HYDROCHLORIDE:Bipolar disorder:0.66), (NEOSTIGMINE:Bipolar disorder:0.57), (NINTEDANIB:Bipolar disorder:0.74), (NIRAPARIB:Bipolar disorder:0.39), (NOREPINEPHRINE BITARTRATE:Bipolar disorder:0.2), (NOREPINEPHRINE\NOREPINEPHRINE BITARTRATE:Bipolar disorder:0.25), (OCTREOTIDE:Bipolar disorder:0.41), (OSELTAMIVIR:Bipolar disorder:0.53), (OXITRIPTAN:Bipolar disorder:0.5), (OXYBUTYNIN\OXYBUTYNIN CHLORIDE:Bipolar disorder:0.57), (PAMIDRONATE DISODIUM:Bipolar disorder:0.74), (PASIREOTIDE:Bipolar disorder:0.15), (PATIROMER:Bipolar disorder:0.44), (PAZOPANIB HYDROCHLORIDE:Bipolar disorder:2.15), (PHENOBARBITAL SODIUM:Bipolar disorder:0.22), (PIMECROLIMUS:Bipolar disorder:0.46), (PIPOTIAZINE PALMITATE:Bipolar disorder:0.01), (PIRACETAM:Bipolar disorder:0.14), (PIRIBEDIL:Bipolar disorder:0.18), (PNEUMOCOCCAL VACCINE, POLYVALENT 23:Bipolar disorder:0.18), (POLYETHYLENE GLYCOL 3350\POTASSIUM CHLORIDE\SODIUM BICARBONATE\SODIUM CHLORIDE\SODIUM SULFATE ANHYDROUS:Bipolar disorder:0.19), (PONATINIB:Bipolar disorder:1.35), (POTASSIUM IODIDE:Bipolar disorder:0.22), (PRAMLINTIDE ACETATE:Bipolar disorder:0.08), (PREDNISOLONE ACETATE:Bipolar disorder:0.99), (PREDNISOLONE SODIUM PHOSPHATE:Bipolar disorder:0.75), (PRISTINAMYCIN:Bipolar disorder:0.06), (PROTRIPTYLINE HYDROCHLORIDE:Bipolar disorder:1.26), (RETINOL:Bipolar disorder:1.21), (RILMENIDINE PHOSPHATE:Bipolar disorder:0.35), (RILPIVIRINE HYDROCHLORIDE:Bipolar disorder:0.68), (ROFECOXIB:Bipolar disorder:0.21), (RUFINAMIDE:Bipolar disorder:0.24), (SACUBITRIL\VALSARTAN:Bipolar disorder:0.64), (SALMETEROL:Bipolar disorder:0.77), (SELEXIPAG:Bipolar disorder:0.16), (SERTINDOLE:Bipolar disorder:0.83), (SEVELAMER HYDROCHLORIDE:Bipolar disorder:0.03), (SILVER SULFADIAZINE:Bipolar disorder:0.31), (SODIUM PHOSPHATE, DIBASIC\SODIUM PHOSPHATE, MONOBASIC:Bipolar disorder:0.33), (SOTALOL HYDROCHLORIDE:Bipolar disorder:0.26), (TAMOXIFEN CITRATE:Bipolar disorder:0.12), (TASIMELTEON:Bipolar disorder:0.77), (TAURINE:Bipolar disorder:0), (TERBUTALINE:Bipolar disorder:1.74), (THALIDOMIDE:Bipolar disorder:1.51), (THEOPHYLLINE:Bipolar disorder:0.24), (TIAPRIDE:Bipolar disorder:0.62), (TIGECYCLINE:Bipolar disorder:0.06), (TIMOLOL MALEATE:Bipolar disorder:0.61), (TINZAPARIN:Bipolar disorder:0.21), (TRIPTORELIN PAMOATE:Bipolar disorder:0.26), (TROPINE BENZYLATE:Bipolar disorder:0.02), (TRYPTOPHAN:Bipolar disorder:0.03), (VALGANCICLOVIR HYDROCHLORIDE:Bipolar disorder:2.35), (ZICONOTIDE ACETATE:Bipolar disorder:0.12), (ZILEUTON:Bipolar disorder:0.57), (ZUCLOPENTHIXOL HYDROCHLORIDE:Bipolar disorder:0.26), (ACARBOSE:Bipolar I disorder:0.2), (ACETAMINOPHEN\OXYCODONE\OXYCODONE HYDROCHLORIDE:Bipolar I disorder:0.15), (ALFACALCIDOL:Bipolar I disorder:0.26), (AMANTADINE:Bipolar I disorder:0.49), (ATOMOXETINE:Bipolar I disorder:0.18), (AZITHROMYCIN DIHYDRATE:Bipolar I disorder:0.08), (BISACODYL:Bipolar I disorder:0.23), (BLONANSERIN:Bipolar I disorder:0.05), (BUPRENORPHINE\NALOXONE:Bipolar I disorder:0.11), (CANAGLIFLOZIN\METFORMIN HYDROCHLORIDE:Bipolar I disorder:0.02), (CHOLESTYRAMINE:Bipolar I disorder:0.63), (CLOTRIMAZOLE:Bipolar I disorder:0.31), (CODEINE:Bipolar I disorder:0.02), (CORTISONE\HYDROCORTISONE:Bipolar I disorder:0.37), (DACLIZUMAB:Bipolar I disorder:0.23), (DALFAMPRIDINE:Bipolar I disorder:0.37), (DASATINIB:Bipolar I disorder:0.39), (DEVICE:Bipolar I disorder:0.81), (DIMENHYDRINATE:Bipolar I disorder:0.22), (DOCUSATE:Bipolar I disorder:0.01), (DOFETILIDE:Bipolar I disorder:0.13), (DOXAZOSIN MESYLATE:Bipolar I disorder:0.2), (DOXEPIN HYDROCHLORIDE:Bipolar I disorder:0.11), (DUTASTERIDE:Bipolar I disorder:0.78), (ELETRIPTAN HYDROBROMIDE:Bipolar I disorder:1.08), (ENALAPRIL MALEATE:Bipolar I disorder:0.5), (ESTROGENS, CONJUGATED\MEDROXYPROGESTERONE ACETATE:Bipolar I disorder:0.52), (EVEROLIMUS:Bipolar I disorder:0.07), (EVOLOCUMAB:Bipolar I disorder:1.15), (EZETIMIBE\SIMVASTATIN:Bipolar I disorder:0.41), (FENTANYL CITRATE:Bipolar I disorder:0.08), (FESOTERODINE FUMARATE:Bipolar I disorder:0.51), (FLECAINIDE:Bipolar I disorder:0.48), (FLUDROCORTISONE ACETATE:Bipolar I disorder:0.11), (FLUTICASONE FUROATE\VILANTEROL TRIFENATATE:Bipolar I disorder:0.14), (HEPARIN SODIUM:Bipolar I disorder:0.41), (HYDRALAZINE HYDROCHLORIDE:Bipolar I disorder:0.16), (HYDROMORPHONE:Bipolar I disorder:0.02), (HYDROMORPHONE HYDROCHLORIDE:Bipolar I disorder:0.62), (IBRUTINIB:Bipolar I disorder:0.9), (IMIPRAMINE HYDROCHLORIDE:Bipolar I disorder:0.16), (INDOMETHACIN:Bipolar I disorder:0.07), (IPRATROPIUM:Bipolar I disorder:0.12), (ISOSORBIDE:Bipolar I disorder:0.39), (LEVALBUTEROL HYDROCHLORIDE:Bipolar I disorder:0.16), (LEVOFLOXACIN:Bipolar I disorder:0.13), (LINAGLIPTIN:Bipolar I disorder:0.63), (METAXALONE:Bipolar I disorder:1.05), (METHENAMINE HIPPURATE:Bipolar I disorder:0.09), (METHYLPREDNISOLONE ACETATE:Bipolar I disorder:0.26), (NADOLOL:Bipolar I disorder:0.11), (NALOXONE HYDROCHLORIDE\OXYCODONE HYDROCHLORIDE:Bipolar I disorder:0.4), (PEGINTERFERON BETA-1A:Bipolar I disorder:0.03), (PENICILLIN:Bipolar I disorder:0.23), (PITAVASTATIN CALCIUM:Bipolar I disorder:0.1), (PROGESTERONE:Bipolar I disorder:0.06), (RABEPRAZOLE:Bipolar I disorder:0.2), (RIVASTIGMINE:Bipolar I disorder:0.45), (SOTALOL:Bipolar I disorder:0.64), (TERIFLUNOMIDE:Bipolar I disorder:0.27), (TESTOSTERONE:Bipolar I disorder:0.43), (TETRACYCLINE:Bipolar I disorder:0.14), (THALIDOMIDE:Bipolar I disorder:0.01), (THYROID, PORCINE:Bipolar I disorder:0.25), (TOCILIZUMAB:Bipolar I disorder:0.47), (TORSEMIDE:Bipolar I disorder:0.15), (ZINC:Bipolar I disorder:0.25), (ZONISAMIDE:Bipolar I disorder:0.31), (BUPRENORPHINE HYDROCHLORIDE\NALOXONE HYDROCHLORIDE:Bipolar II disorder:0.62), (DEXLANSOPRAZOLE:Bipolar II disorder:0.1), (GUANFACINE:Bipolar II disorder:0.03), (POTASSIUM:Bipolar II disorder:0.05), (PROGESTERONE:Bipolar II disorder:0.28), (ACENOCOUMAROL:Bladder cancer:0.51), (ACETAMINOPHEN\BUTALBITAL\CAFFEINE:Bladder cancer:0.04), (ANASTROZOLE:Bladder cancer:1.66), (BUPROPION:Bladder cancer:0.19), (CALCIUM ACETATE:Bladder cancer:0.16), (CALCIUM\VITAMIN D:Bladder cancer:0.56), (CARFILZOMIB:Bladder cancer:0.49), (CERITINIB:Bladder cancer:0.13), (CHOLESTYRAMINE:Bladder cancer:0.39), (CLOTRIMAZOLE:Bladder cancer:0.27), (CORTISONE\HYDROCORTISONE:Bladder cancer:0.43), (CYCLOBENZAPRINE:Bladder cancer:0.2), (DABRAFENIB:Bladder cancer:0.27), (DARATUMUMAB:Bladder cancer:0.01), (DESLORATADINE:Bladder cancer:0.02), (DIVALPROEX SODIUM:Bladder cancer:0.01), (DRONABINOL:Bladder cancer:0.21), (DULOXETINE:Bladder cancer:0.11), (ESZOPICLONE:Bladder cancer:0.21), (EVOLOCUMAB:Bladder cancer:0.44), (EXEMESTANE:Bladder cancer:0.57), (GEFITINIB:Bladder cancer:0.21), (HYDROCHLOROTHIAZIDE\IRBESARTAN:Bladder cancer:0.22), (HYDROXYUREA:Bladder cancer:0.39), (IMATINIB MESYLATE:Bladder cancer:0.75), (INDAPAMIDE:Bladder cancer:0.12), (INSULIN ASPART:Bladder cancer:0.88), (ISOSORBIDE:Bladder cancer:0.19), (LEDIPASVIR\SOFOSBUVIR:Bladder cancer:0.32), (LENVATINIB:Bladder cancer:0), (LERCANIDIPINE HYDROCHLORIDE:Bladder cancer:0.21), (LETROZOLE:Bladder cancer:0.98), (LEVALBUTEROL HYDROCHLORIDE:Bladder cancer:0.12), (LEVOLEUCOVORIN CALCIUM:Bladder cancer:0.3), (LIDOCAINE\PRILOCAINE:Bladder cancer:0.4), (LINAGLIPTIN:Bladder cancer:0.43), (MELATONIN:Bladder cancer:0.23), (METAXALONE:Bladder cancer:0.14), (MOXIFLOXACIN HYDROCHLORIDE:Bladder cancer:0.4), (NIRAPARIB:Bladder cancer:0.01), (OMEGA-3-ACID ETHYL ESTERS:Bladder cancer:0.24), (OXCARBAZEPINE:Bladder cancer:0.02), (PANTOPRAZOLE MAGNESIUM:Bladder cancer:0.22), (PEMETREXED:Bladder cancer:0.41), (PENICILLIN:Bladder cancer:0.01), (PERTUZUMAB:Bladder cancer:0.34), (PONATINIB:Bladder cancer:0.1), (PRAMIPEXOLE DIHYDROCHLORIDE:Bladder cancer:0.2), (PROMETHAZINE\PROMETHAZINE HYDROCHLORIDE:Bladder cancer:0.38), (PYRAZINAMIDE:Bladder cancer:0.67), (RANOLAZINE:Bladder cancer:0.52), (RIOCIGUAT:Bladder cancer:0.15), (SACUBITRIL\VALSARTAN:Bladder cancer:0.85), (SODIUM OXYBATE:Bladder cancer:0.14), (TERIFLUNOMIDE:Bladder cancer:0.25), (THALIDOMIDE:Bladder cancer:0.96), (TIMOLOL:Bladder cancer:0.05), (TIZANIDINE HYDROCHLORIDE:Bladder cancer:0.16), (TOCILIZUMAB:Bladder cancer:0.4), (TRAMETINIB:Bladder cancer:0.27), (TRIAMCINOLONE ACETONIDE:Bladder cancer:0.02), (UMECLIDINIUM BROMIDE\VILANTEROL TRIFENATATE:Bladder cancer:0.16), (VARENICLINE TARTRATE:Bladder cancer:0.1), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Bladder disorder:0.27), (.ALPHA.1-PROTEINASE INHIBITOR HUMAN:Bladder disorder:0.05), (AGALSIDASE BETA:Bladder disorder:0.55), (ALBIGLUTIDE:Bladder disorder:0.25), (ALFACALCIDOL:Bladder disorder:0.16), (ALISKIREN HEMIFUMARATE:Bladder disorder:0.75), (AMISULPRIDE:Bladder disorder:0.22), (AMLODIPINE BESYLATE\ATORVASTATIN CALCIUM:Bladder disorder:0.19), (AMLODIPINE BESYLATE\HYDROCHLOROTHIAZIDE\OLMESARTAN MEDOXOMIL:Bladder disorder:0.55), (AMLODIPINE BESYLATE\HYDROCHLOROTHIAZIDE\VALSARTAN:Bladder disorder:0.35), (AMLODIPINE BESYLATE\OLMESARTAN MEDOXOMIL:Bladder disorder:0.45), (AMLODIPINE\VALSARTAN:Bladder disorder:0.35), (AMMONIUM LACTATE:Bladder disorder:0.47), (ANTIHISTAMINES NOS:Bladder disorder:0.08), (ARIPIPRAZOLE LAUROXIL:Bladder disorder:0.21), (BETAMETHASONE DIPROPIONATE\CALCIPOTRIENE\CALCIPOTRIENE HYDRATE:Bladder disorder:0.52), (BETAMETHASONE DIPROPIONATE\CLOTRIMAZOLE:Bladder disorder:0.09), (BETAMETHASONE\CALCIPOTRIENE:Bladder disorder:0), (BORTEZOMIB:Bladder disorder:0.39), (BUDESONIDE\FORMOTEROL FUMARATE:Bladder disorder:0.7), (CALCIPOTRIENE:Bladder disorder:0.2), (CALCITONIN SALMON:Bladder disorder:0.45), (CALCIUM POLYCARBOPHIL:Bladder disorder:0.03), (CALCIUM\MAGNESIUM:Bladder disorder:0.63), (CANAGLIFLOZIN\METFORMIN HYDROCHLORIDE:Bladder disorder:1), (CANAKINUMAB:Bladder disorder:0.01), (CANDESARTAN CILEXETIL\HYDROCHLOROTHIAZIDE:Bladder disorder:1.38), (CAPTOPRIL:Bladder disorder:0.93), (CEFTRIAXONE SODIUM:Bladder disorder:0.14), (CEVIMELINE HYDROCHLORIDE:Bladder disorder:0.02), (CHLORPROMAZINE:Bladder disorder:0.04), (CHOLESTYRAMINE:Bladder disorder:1.56), (CICLESONIDE:Bladder disorder:0.39), (CLIKSTAR:Bladder disorder:0.14), (CLINDAMYCIN:Bladder disorder:0.92), (COD LIVER OIL:Bladder disorder:0.53), (CYPROHEPTADINE:Bladder disorder:0.01), (DAPAGLIFLOZIN PROPANEDIOL:Bladder disorder:1.25), (DASATINIB:Bladder disorder:1.08), (DEFLAZACORT:Bladder disorder:0.79), (DEXTROAMPHETAMINE SULFATE:Bladder disorder:0.34), (DORZOLAMIDE HYDROCHLORIDE\TIMOLOL MALEATE:Bladder disorder:0.05), (DOXEPIN HYDROCHLORIDE:Bladder disorder:0.75), (DRONABINOL:Bladder disorder:0.84), (EDOXABAN TOSYLATE:Bladder disorder:0.82), (EMPAGLIFLOZIN\LINAGLIPTIN:Bladder disorder:1.22), (ENOXAPARIN:Bladder disorder:1.38), (EPLERENONE:Bladder disorder:0.77), (ESTRIOL:Bladder disorder:0.05), (EVEROLIMUS:Bladder disorder:0.96), (FEBUXOSTAT:Bladder disorder:0.74), (FLUTICASONE FUROATE:Bladder disorder:0.69), (FLUVOXAMINE MALEATE:Bladder disorder:0.41), (FORMOTEROL FUMARATE:Bladder disorder:0.5), (FORMOTEROL FUMARATE\MOMETASONE FUROATE:Bladder disorder:0.09), (FUSIDIC ACID:Bladder disorder:0.61), (GLYCOPYRRONIUM\INDACATEROL:Bladder disorder:0.59), (GUANFACINE:Bladder disorder:0.27), (GUANFACINE HYDROCHLORIDE:Bladder disorder:0.07), (HYDROXYCHLOROQUINE DIPHOSPHATE:Bladder disorder:0.25), (ILOPROST:Bladder disorder:0.38), (IMATINIB MESYLATE:Bladder disorder:0.77), (INDAPAMIDE\PERINDOPRIL:Bladder disorder:0.37), (INSULIN BEEF:Bladder disorder:0.25), (LABETALOL HYDROCHLORIDE:Bladder disorder:0.61), (LACTOBACILLUS RHAMNOSUS:Bladder disorder:0.1), (LANREOTIDE ACETATE:Bladder disorder:0.06), (LEDIPASVIR\SOFOSBUVIR:Bladder disorder:0.87), (LERCANIDIPINE HYDROCHLORIDE:Bladder disorder:0.8), (LEVOMEFOLATE CALCIUM:Bladder disorder:0.67), (LEVOMILNACIPRAN HYDROCHLORIDE:Bladder disorder:0.07), (LIDOCAINE\PRILOCAINE:Bladder disorder:0.18), (LURASIDONE HYDROCHLORIDE:Bladder disorder:0.35), (METHYLPREDNISOLONE ACETATE:Bladder disorder:1.24), (MIDODRINE HYDROCHLORIDE:Bladder disorder:0.2), (NALOXONE HYDROCHLORIDE\OXYCODONE HYDROCHLORIDE:Bladder disorder:0.97), (NILOTINIB:Bladder disorder:0.94), (NORETHINDRONE:Bladder disorder:0.15), (ORPHENADRINE:Bladder disorder:0.15), (PANTOPRAZOLE MAGNESIUM:Bladder disorder:1.34), (PAZOPANIB HYDROCHLORIDE:Bladder disorder:0.46), (PEGINTERFERON BETA-1A:Bladder disorder:1.09), (PEMETREXED DISODIUM:Bladder disorder:0.11), (PIRFENIDONE:Bladder disorder:0.02), (PRAZOSIN HYDROCHLORIDE:Bladder disorder:0.42), (PROPAFENONE:Bladder disorder:0.04), (PROPAFENONE HYDROCHLORIDE:Bladder disorder:0.73), (PROTRIPTYLINE HYDROCHLORIDE:Bladder disorder:0.25), (SALMETEROL:Bladder disorder:0.15), (SENNOSIDES:Bladder disorder:0.63), (SULINDAC:Bladder disorder:0.14), (TERBUTALINE SULFATE:Bladder disorder:1.01), (VALGANCICLOVIR HYDROCHLORIDE:Bladder disorder:0.41), (ZIPRASIDONE HYDROCHLORIDE\ZIPRASIDONE MESYLATE:Bladder disorder:0.32), (PYRAZINAMIDE:Bladder neoplasm:0.57), (CALCIUM CARBONATE\CHOLECALCIFEROL:Bladder pain:0.01), (CITALOPRAM HYDROBROMIDE:Bladder pain:0.45), (DOCUSATE SODIUM:Bladder pain:0.23), (EVOLOCUMAB:Bladder pain:0.03), (MELOXICAM:Bladder pain:0.08), (METHYLPREDNISOLONE SODIUM SUCCINATE:Bladder pain:0.25), (PANTOPRAZOLE:Bladder pain:0.07), (SACUBITRIL\VALSARTAN:Bladder pain:0.06), (TERIFLUNOMIDE:Bladder pain:0.79), (TRAZODONE HYDROCHLORIDE:Bladder pain:0.41), (VARENICLINE TARTRATE:Bladder pain:0.03), (AZITHROMYCIN ANHYDROUS:Bladder spasm:0.27), (BENAZEPRIL HYDROCHLORIDE:Bladder spasm:0.61), (BISACODYL OR DOCUSATE SODIUM:Bladder spasm:0.58), (BISOPROLOL FUMARATE:Bladder spasm:0.38), (BUPRENORPHINE HYDROCHLORIDE:Bladder spasm:0.04), (BUPRENORPHINE HYDROCHLORIDE\NALOXONE HYDROCHLORIDE:Bladder spasm:0.42), (CALCITRIOL:Bladder spasm:0.44), (CALCIUM ACETATE:Bladder spasm:0.21), (CALCIUM CARBONATE\CHOLECALCIFEROL:Bladder spasm:1.04), (CANDESARTAN:Bladder spasm:0.34), (CANDESARTAN CILEXETIL:Bladder spasm:0.43), (CIMETIDINE:Bladder spasm:0), (CINACALCET HYDROCHLORIDE:Bladder spasm:0.06), (CLINDAMYCIN\CLINDAMYCIN PHOSPHATE:Bladder spasm:0.85), (CLOZAPINE:Bladder spasm:0.12), (CORTISONE\HYDROCORTISONE:Bladder spasm:0.41), (DACLIZUMAB:Bladder spasm:0.23), (DASATINIB:Bladder spasm:0.44), (DEVICE:Bladder spasm:0.16), (DIMENHYDRINATE:Bladder spasm:0.31), (ENALAPRIL:Bladder spasm:1.47), (ENALAPRIL MALEATE:Bladder spasm:0.43), (ESTROGENS, CONJUGATED\MEDROXYPROGESTERONE ACETATE:Bladder spasm:0.11), (EVEROLIMUS:Bladder spasm:0.16), (EVOLOCUMAB:Bladder spasm:0.81), (HERBALS\TURMERIC:Bladder spasm:0.03), (INDOMETHACIN:Bladder spasm:0.48), (IRBESARTAN:Bladder spasm:0.17), (ISOSORBIDE:Bladder spasm:0.17), (LABETALOL\LABETALOL HYDROCHLORIDE:Bladder spasm:0.23), (LACOSAMIDE:Bladder spasm:0.11), (LATANOPROST:Bladder spasm:0.3), (LETROZOLE:Bladder spasm:0.24), (LISDEXAMFETAMINE DIMESYLATE:Bladder spasm:0.84), (MEDROXYPROGESTERONE ACETATE:Bladder spasm:0.09), (METHADONE HYDROCHLORIDE:Bladder spasm:0.18), (METHYLPREDNISOLONE SODIUM SUCCINATE:Bladder spasm:1.21), (MOXIFLOXACIN HYDROCHLORIDE:Bladder spasm:0.58), (NADOLOL:Bladder spasm:0.07), (NEBIVOLOL:Bladder spasm:0.07), (NILOTINIB:Bladder spasm:0.31), (OMEGA-3-ACID ETHYL ESTERS:Bladder spasm:0.29), (OXCARBAZEPINE:Bladder spasm:1.21), (PEGFILGRASTIM:Bladder spasm:0.24), (PEGINTERFERON BETA-1A:Bladder spasm:0.12), (PENICILLIN:Bladder spasm:0.19), (PONATINIB:Bladder spasm:0.13), (PRIMIDONE:Bladder spasm:0.43), (PROPRANOLOL\PROPRANOLOL HYDROCHLORIDE:Bladder spasm:0.01), (RANOLAZINE:Bladder spasm:0.61), (RIZATRIPTAN BENZOATE:Bladder spasm:0.69), (ROFLUMILAST:Bladder spasm:0.04), (SACUBITRIL\VALSARTAN:Bladder spasm:0.58), (SECUKINUMAB:Bladder spasm:0.03), (SUMATRIPTAN:Bladder spasm:0.32), (TOFACITINIB CITRATE:Bladder spasm:0.31), (IBRUTINIB:Bladder transitional cell carcinoma:0.07), (CARBOXYMETHYLCELLULOSE SODIUM\GLYCERIN\POLYSORBATE 80:Blepharitis:0.65), (NAPROXEN:Blepharitis:0.51), (ACETAMINOPHEN\CODEINE PHOSPHATE:Blood calcium:0.25), (BUMETANIDE:Blood calcium:0.24), (BUSPIRONE HYDROCHLORIDE:Blood calcium:0.11), (CERTOLIZUMAB PEGOL:Blood calcium:0.67), (CITALOPRAM HYDROBROMIDE:Blood calcium:0.68), (COLCHICINE:Blood calcium:0.01), (DASATINIB:Blood calcium:0.05), (DICLOFENAC:Blood calcium:0.11), (DIVALPROEX SODIUM:Blood calcium:0.22), (ENOXAPARIN:Blood calcium:0.09), (EVEROLIMUS:Blood calcium:0.65), (EVOLOCUMAB:Blood calcium:0.54), (HYDROCHLOROTHIAZIDE\VALSARTAN:Blood calcium:0.06), (INSULIN DETEMIR:Blood calcium:0.11), (INSULIN HUMAN:Blood calcium:0.36), (NAPROXEN:Blood calcium:0.84), (NEBIVOLOL:Blood calcium:0.08), (POLYETHYLENE GLYCOLS:Blood calcium:0.13), (POTASSIUM:Blood calcium:0.77), (PRAVASTATIN:Blood calcium:0.4), (QUETIAPINE FUMARATE:Blood calcium:0.07), (RANOLAZINE:Blood calcium:0.48), (SACUBITRIL\VALSARTAN:Blood calcium:0.63), (TERIFLUNOMIDE:Blood calcium:0.58), (TRAMADOL HYDROCHLORIDE:Blood calcium:0.02), (VERAPAMIL HYDROCHLORIDE:Blood calcium:0.55), (RISEDRONATE SODIUM:Blood calcium abnormal:0.08), (AMIODARONE HYDROCHLORIDE:Blood calcium decreased:0.55), (CANDESARTAN CILEXETIL:Blood calcium decreased:0.55), (CHOLESTYRAMINE:Blood calcium decreased:0.06), (COLCHICINE:Blood calcium decreased:0.36), (DASATINIB:Blood calcium decreased:0.82), (DEXLANSOPRAZOLE:Blood calcium decreased:1.04), (DIPHENHYDRAMINE:Blood calcium decreased:0.84), (DIVALPROEX SODIUM:Blood calcium decreased:0.32), (DOFETILIDE:Blood calcium decreased:0.17), (DOXAZOSIN MESYLATE:Blood calcium decreased:0.12), (DUTASTERIDE:Blood calcium decreased:0.15), (ELETRIPTAN HYDROBROMIDE:Blood calcium decreased:0.09), (ESZOPICLONE:Blood calcium decreased:0.1), (EVOLOCUMAB:Blood calcium decreased:0.7), (FESOTERODINE FUMARATE:Blood calcium decreased:0.02), (FINGOLIMOD HYDROCHLORIDE:Blood calcium decreased:0.08), (FLUTICASONE:Blood calcium decreased:0.11), (GLYBURIDE:Blood calcium decreased:0.2), (ILOPROST:Blood calcium decreased:0.14), (ISONIAZID:Blood calcium decreased:0.44), (ISOSORBIDE MONONITRATE:Blood calcium decreased:0.42), (LEDIPASVIR\SOFOSBUVIR:Blood calcium decreased:0.1), (LEVALBUTEROL HYDROCHLORIDE:Blood calcium decreased:0.12), (LIDOCAINE:Blood calcium decreased:0.6), (LIDOCAINE\PRILOCAINE:Blood calcium decreased:0.02), (METHENAMINE HIPPURATE:Blood calcium decreased:0), (NEBIVOLOL:Blood calcium decreased:0.39), (NITROFURANTOIN:Blood calcium decreased:0.1), (NITROGLYCERIN:Blood calcium decreased:0.36), (OXCARBAZEPINE:Blood calcium decreased:0.28), (PANTOPRAZOLE MAGNESIUM:Blood calcium decreased:0.34), (PRIMIDONE:Blood calcium decreased:0.2), (PROMETHAZINE\PROMETHAZINE HYDROCHLORIDE:Blood calcium decreased:0.45), (RABEPRAZOLE SODIUM:Blood calcium decreased:0.47), (RANOLAZINE:Blood calcium decreased:0.93), (SECUKINUMAB:Blood calcium decreased:0.01), (SULFAMETHOXAZOLE:Blood calcium decreased:0.18), (TERIFLUNOMIDE:Blood calcium decreased:0.64), (TIMOLOL:Blood calcium decreased:0.16), (TOCILIZUMAB:Blood calcium decreased:0.51), (TRIAMTERENE:Blood calcium decreased:0.09), (VALGANCICLOVIR HYDROCHLORIDE:Blood calcium decreased:0.03), (VARENICLINE TARTRATE:Blood calcium decreased:0.06), (.ALPHA.-TOCOPHEROL\VITAMINS:Blood cholesterol:0.09), (.BETA.-CAROTENE:Blood cholesterol:0), (ABACAVIR SULFATE\LAMIVUDINE:Blood cholesterol:0.04), (ABOBOTULINUMTOXINA:Blood cholesterol:0.62), (ACEBUTOLOL HYDROCHLORIDE:Blood cholesterol:0.56), (ACETAMINOPHEN\CAFFEINE\CARISOPRODOL\DICLOFENAC SODIUM:Blood cholesterol:0.53), (ACETAMINOPHEN\METHOCARBAMOL:Blood cholesterol:0.5), (ACETYLCARNITINE HYDROCHLORIDE:Blood cholesterol:0.27), (ALBUTEROL\IPRATROPIUM BROMIDE:Blood cholesterol:0.05), (ALISKIREN HEMIFUMARATE\HYDROCHLOROTHIAZIDE:Blood cholesterol:0.45), (ALUMINUM HYDROXIDE\MAGNESIUM HYDROXIDE\SIMETHICONE:Blood cholesterol:0.29), (AMILORIDE HYDROCHLORIDE\FUROSEMIDE:Blood cholesterol:0.14), (AMINO ACIDS\DEXTROSE\ELECTROLYTES NOS:Blood cholesterol:0.85), (AMPICILLIN:Blood cholesterol:0.86), (AMPICILLIN SODIUM\SULBACTAM SODIUM:Blood cholesterol:0.26), (ANTIHISTAMINES NOS:Blood cholesterol:1.18), (ARIPIPRAZOLE LAUROXIL:Blood cholesterol:1.07), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Blood cholesterol:0.32), (ASPERGILLUS NIGER.ALPHA.-GALACTOSIDASE:Blood cholesterol:0.4), (ASPIRIN\CAFFEINE\SALICYLAMIDE:Blood cholesterol:0.03), (ASPIRIN\MAGNESIUM OXIDE:Blood cholesterol:0.27), (ASUNAPREVIR:Blood cholesterol:0.02), (ATROPINE SULFATE:Blood cholesterol:0.13), (ATROPINE SULFATE\HYOSCYAMINE SULFATE\PHENOBARBITAL\SCOPOLAMINE HYDROBROMIDE:Blood cholesterol:0.06), (AZITHROMYCIN MONOHYDRATE:Blood cholesterol:0.62), (BACITRACIN:Blood cholesterol:1.33), (BELATACEPT:Blood cholesterol:0.36), (BENZOCAINE:Blood cholesterol:0.25), (BLINATUMOMAB:Blood cholesterol:0.59), (BOCEPREVIR:Blood cholesterol:0.53), (BOTULINUM TOXIN TYPE A:Blood cholesterol:0.42), (BROTIZOLAM:Blood cholesterol:0.15), (BUPIVACAINE HYDROCHLORIDE:Blood cholesterol:0.04), (BUPRENORPHINE\NALOXONE:Blood cholesterol:0.12), (BUTALBITAL:Blood cholesterol:0.43), (CABOZANTINIB S-MALATE:Blood cholesterol:0.55), (CALCIUM CARBONATE\ERGOCALCIFEROL:Blood cholesterol:0.11), (CALCIUM CARBONATE\ETIDRONATE DISODIUM:Blood cholesterol:0.03), (CALCIUM PANTOTHENATE\CYANOCOBALAMIN\NIACINAMIDE\PYRIDOXINE HYDROCHLORIDE\RIBOFLAVIN\THIAMINE MONONITRATE:Blood cholesterol:0.21), (CALCIUM\VITAMIN D\VITAMIN K5:Blood cholesterol:0.3), (CARBIMAZOLE:Blood cholesterol:0.5), (CEFAZOLIN SODIUM:Blood cholesterol:0.42), (CEFOTAXIME SODIUM:Blood cholesterol:0.45), (CEFPODOXIME PROXETIL:Blood cholesterol:0.13), (CEFPROZIL:Blood cholesterol:0.28), (CERITINIB:Blood cholesterol:0.47), (CERIVASTATIN SODIUM:Blood cholesterol:0.59), (CILASTATIN SODIUM\IMIPENEM:Blood cholesterol:1.26), (CURCUMIN:Blood cholesterol:0.68), (DABRAFENIB MESYLATE:Blood cholesterol:0.49), (DACLATASVIR:Blood cholesterol:0.55), (DACLIZUMAB:Blood cholesterol:2.1), (DANAZOL:Blood cholesterol:1.39), (DANTROLENE SODIUM:Blood cholesterol:0.35), (DEXAMETHASONE ACETATE:Blood cholesterol:0.46), (DEXAMETHASONE SODIUM PHOSPHATE:Blood cholesterol:0.23), (DEXMETHYLPHENIDATE HYDROCHLORIDE:Blood cholesterol:0.27), (DIFLUNISAL:Blood cholesterol:0.68), (DIPYRONE\PROMETHAZINE HYDROCHLORIDE:Blood cholesterol:0.83), (DOLASETRON MESYLATE:Blood cholesterol:0.93), (ELBASVIR\GRAZOPREVIR:Blood cholesterol:0.66), (ELTROMBOPAG OLAMINE:Blood cholesterol:0.56), (ENALAPRIL MALEATE\LERCANIDIPINE HYDROCHLORIDE:Blood cholesterol:0.06), (ENSULIZOLE\EUCERIN\OCTINOXATE\OCTISALATE\TITANIUM DIOXIDE\ZINC OXIDE:Blood cholesterol:0.19), (EPIRUBICIN:Blood cholesterol:0.31), (ERIBULIN:Blood cholesterol:0.01), (ESOMEPRAZOLE SODIUM:Blood cholesterol:0.37), (ESTRADIOL BENZOATE:Blood cholesterol:0.06), (ESTROGENS\METHYLTESTOSTERONE:Blood cholesterol:0), (FENOTEROL HYDROBROMIDE\IPRATROPIUM BROMIDE:Blood cholesterol:0.62), (FERROUS SULFATE\GLYCINE:Blood cholesterol:0.1), (FLURAZEPAM\FLURAZEPAM HYDROCHLORIDE:Blood cholesterol:0.23), (FLURBIPROFEN:Blood cholesterol:0.02), (FORMOTEROL:Blood cholesterol:1.63), (GIMERACIL\OTERACIL\TEGAFUR:Blood cholesterol:0.13), (GLUTAMINE:Blood cholesterol:0.29), (GLYCOPYRROLATE\INDACATEROL MALEATE:Blood cholesterol:0.93), (GLYCOPYRRONIUM\INDACATEROL:Blood cholesterol:2.24), (HALCINONIDE:Blood cholesterol:0.17), (HALOPERIDOL DECANOATE:Blood cholesterol:0.09), (HEPARIN CALCIUM:Blood cholesterol:0.13), (HERBALS\MINERALS\VITAMINS:Blood cholesterol:0.1), (HORSE CHESTNUT:Blood cholesterol:0.45), (HYDRAZINE SULFATE:Blood cholesterol:0.09), (HYDROXYCITRIC ACID:Blood cholesterol:0.03), (HYDROXYPROPYL CELLULOSE:Blood cholesterol:0.18), (HYOSCYAMINE SULFATE\METHENAMINE\METHYLENE BLUE\PHENYL SALICYLATE\SODIUM PHOSPHATE, MONOBASIC, MONOHYDRATE:Blood cholesterol:0.55), (INTERFERON:Blood cholesterol:1.74), (INTERFERON ALFA-2A:Blood cholesterol:1.37), (INTERFERON GAMMA-1B:Blood cholesterol:0.56), (LACTOBACILLUS ACIDOPHILUS\LACTOBACILLUS DELBRUECKII SUBSP. BULGARICUS:Blood cholesterol:0.42), (LANREOTIDE:Blood cholesterol:0.98), (LENVATINIB:Blood cholesterol:0.56), (LEVOBUNOLOL:Blood cholesterol:0.05), (LEVOLEUCOVORIN CALCIUM:Blood cholesterol:0.14), (LUTETIUM LU-177:Blood cholesterol:0.91), (MECLOZINE DIHYDROCHLORIDE:Blood cholesterol:0.01), (MENTHOL\ZINC OXIDE:Blood cholesterol:0.08), (METFORMIN HYDROCHLORIDE\ROSIGLITAZONE MALEATE:Blood cholesterol:1.14), (METHENAMINE MANDELATE:Blood cholesterol:0.62), (NALOXONE HYDROCHLORIDE\TILIDINE HYDROCHLORIDE:Blood cholesterol:1.39), (NARATRIPTAN HYDROCHLORIDE:Blood cholesterol:1.06), (NEOMYCIN:Blood cholesterol:0.61), (NICARDIPINE:Blood cholesterol:0.5), (NOREPINEPHRINE BITARTRATE:Blood cholesterol:0.49), (OMEPRAZOLE SODIUM:Blood cholesterol:1.06), (OPIPRAMOL:Blood cholesterol:0.17), (OXAPROZIN:Blood cholesterol:0.65), (OXYMORPHONE:Blood cholesterol:0.37), (PAPAYA:Blood cholesterol:0.31), (PARATHYROID HORMONE:Blood cholesterol:0.78), (PASIREOTIDE:Blood cholesterol:1.29), (PEMETREXED:Blood cholesterol:0.64), (PENTOSTATIN:Blood cholesterol:0.27), (PHENAZOPYRIDINE HYDROCHLORIDE:Blood cholesterol:0.93), (PHENTERMINE HYDROCHLORIDE:Blood cholesterol:0.08), (POVIDONE:Blood cholesterol:0.22), (PRAMOXINE HYDROCHLORIDE:Blood cholesterol:0.45), (PREDNISOLONE\PREDNISOLONE ACETATE\PREDNISOLONE SODIUM SUCCINATE:Blood cholesterol:0.46), (PROTRIPTYLINE HYDROCHLORIDE:Blood cholesterol:1.3), (PSEUDOEPHEDRINE:Blood cholesterol:0.19), (PYRAZINAMIDE:Blood cholesterol:0.78), (QUINACRINE:Blood cholesterol:0.35), (QUINIDINE GLUCONATE:Blood cholesterol:0.23), (RADIATION THERAPY:Blood cholesterol:1.01), (REGORAFENIB:Blood cholesterol:1.39), (ROFECOXIB:Blood cholesterol:0.3), (SALMETEROL:Blood cholesterol:1.27), (SELEXIPAG:Blood cholesterol:0.8), (SIBUTRAMINE HYDROCHLORIDE:Blood cholesterol:0.55), (SODIUM PHOSPHATE, DIBASIC\SODIUM PHOSPHATE, MONOBASIC:Blood cholesterol:0.79), (SULFACETAMIDE SODIUM\SULFUR:Blood cholesterol:0.11), (TAZAROTENE:Blood cholesterol:0.03), (TEDUGLUTIDE\WATER:Blood cholesterol:0.94), (TELAPREVIR:Blood cholesterol:0.04), (TELOTRISTAT ETHYL:Blood cholesterol:1.12), (TERBUTALINE:Blood cholesterol:0.42), (THEOPHYLLINE:Blood cholesterol:0.36), (TILIDINE:Blood cholesterol:0.01), (TIMOLOL MALEATE\TRAVOPROST:Blood cholesterol:0.82), (TINZAPARIN SODIUM:Blood cholesterol:1.06), (TRAMETINIB DIMETHYL SULFOXIDE:Blood cholesterol:0.46), (TREPROSTINIL DIOLAMINE:Blood cholesterol:0.22), (TRIMIPRAMINE MALEATE:Blood cholesterol:0.6), (TRIPTORELIN PAMOATE:Blood cholesterol:1.07), (UBIQUINOL:Blood cholesterol:0.53), (VALDECOXIB:Blood cholesterol:0.49), (VANDETANIB:Blood cholesterol:0.21), (VINCRISTINE SULFATE:Blood cholesterol:0.03), (VINORELBINE TARTRATE:Blood cholesterol:0.47), (VORAPAXAR SULFATE:Blood cholesterol:0.05), (ZILEUTON:Blood cholesterol:0.96), (.ALPHA.1-PROTEINASE INHIBITOR HUMAN:Blood cholesterol abnormal:1.03), (ABOBOTULINUMTOXINA:Blood cholesterol abnormal:0.23), (ACETAMINOPHEN\CAFFEINE\CARISOPRODOL\DICLOFENAC SODIUM:Blood cholesterol abnormal:0.66), (ACETAMINOPHEN\CAFFEINE\OPIUM:Blood cholesterol abnormal:0.13), (ACTIVATED CHARCOAL:Blood cholesterol abnormal:0.05), (ADO-TRASTUZUMAB EMTANSINE:Blood cholesterol abnormal:0.96), (AGALSIDASE BETA:Blood cholesterol abnormal:1.58), (ALISKIREN HEMIFUMARATE\HYDROCHLOROTHIAZIDE:Blood cholesterol abnormal:0.29), (AMANTADINE:Blood cholesterol abnormal:1.61), (AMINO ACIDS\DEXTROSE\ELECTROLYTES NOS:Blood cholesterol abnormal:0.43), (AMISULPRIDE:Blood cholesterol abnormal:1.02), (AMOXICILLIN\CLARITHROMYCIN\ESOMEPRAZOLE:Blood cholesterol abnormal:0.18), (ANAGRELIDE HYDROCHLORIDE:Blood cholesterol abnormal:0.41), (ANAKINRA:Blood cholesterol abnormal:0.01), (ANTIHISTAMINES NOS:Blood cholesterol abnormal:1), (APREPITANT:Blood cholesterol abnormal:0.44), (ARIPIPRAZOLE LAUROXIL:Blood cholesterol abnormal:0.55), (ASPIRIN\CAFFEINE\SALICYLAMIDE:Blood cholesterol abnormal:0.06), (ASPIRIN\MAGNESIUM OXIDE:Blood cholesterol abnormal:0.09), (ATOVAQUONE:Blood cholesterol abnormal:0.08), (AVANAFIL:Blood cholesterol abnormal:0.06), (AZITHROMYCIN MONOHYDRATE:Blood cholesterol abnormal:0.28), (BACITRACIN ZINC:Blood cholesterol abnormal:0.31), (BALSALAZIDE DISODIUM:Blood cholesterol abnormal:0.42), (BETAMETHASONE DIPROPIONATE\CALCIPOTRIENE:Blood cholesterol abnormal:0.2), (BETAMETHASONE DIPROPIONATE\CALCIPOTRIENE\CALCIPOTRIENE HYDRATE:Blood cholesterol abnormal:1.44), (BETAMETHASONE\CALCIPOTRIENE:Blood cholesterol abnormal:0.77), (BETAXOLOL HYDROCHLORIDE:Blood cholesterol abnormal:0.03), (BETHANECHOL CHLORIDE:Blood cholesterol abnormal:0.6), (BOTULINUM TOXIN TYPE A:Blood cholesterol abnormal:0.08), (BUPRENORPHINE HYDROCHLORIDE\NALOXONE HYDROCHLORIDE:Blood cholesterol abnormal:1.01), (CABOZANTINIB S-MALATE:Blood cholesterol abnormal:0.24), (CALCIUM CARBONATE\ERGOCALCIFEROL\RETINOL:Blood cholesterol abnormal:0.24), (CALCIUM PANTOTHENATECYANOCOBALAMIN\NIACINAMIDE\PYRIDOXINE HYDROCHLORIDE\RIBOFLAVIN\THIAMINE MONONITRATE:Blood cholesterol abnormal:0.18), (CANAKINUMAB:Blood cholesterol abnormal:0.74), (CARBIMAZOLE:Blood cholesterol abnormal:0.26), (CARBOMER:Blood cholesterol abnormal:0.11), (CEFAZOLIN:Blood cholesterol abnormal:0.28), (CHLORHEXIDINE:Blood cholesterol abnormal:0.64), (CHLORPROMAZINE:Blood cholesterol abnormal:0.36), (CILASTATIN SODIUM\IMIPENEM:Blood cholesterol abnormal:0.57), (CORTICOTROPIN:Blood cholesterol abnormal:1.51), (CYPROHEPTADINE HYDROCHLORIDE:Blood cholesterol abnormal:0.37), (DACLIZUMAB:Blood cholesterol abnormal:1.37), (DALTEPARIN:Blood cholesterol abnormal:1.05), (DANTROLENE SODIUM:Blood cholesterol abnormal:0.18), (DIFLUNISAL:Blood cholesterol abnormal:0.51), (DIMETHICONE 410:Blood cholesterol abnormal:0.03), (DIPHENHYDRAMINE HYDROCHLORIDE\LIDOCAINE HYDROCHLORIDE\NYSTATIN:Blood cholesterol abnormal:0.01), (DOLASETRON MESYLATE:Blood cholesterol abnormal:0.59), (DROXIDOPA:Blood cholesterol abnormal:0.53), (ECHINACEA, UNSPECIFIED:Blood cholesterol abnormal:0.04), (ECONAZOLE:Blood cholesterol abnormal:0.85), (ELBASVIR\GRAZOPREVIR:Blood cholesterol abnormal:0.01), (ELOTUZUMAB:Blood cholesterol abnormal:0.06), (ELTROMBOPAG OLAMINE:Blood cholesterol abnormal:0.48), (EMPAGLIFLOZIN\METFORMIN HYDROCHLORIDE:Blood cholesterol abnormal:0.37), (EPIRUBICIN:Blood cholesterol abnormal:0.12), (ESTROPIPATE:Blood cholesterol abnormal:0.13), (ETHINYL ESTRADIOL\NORGESTREL:Blood cholesterol abnormal:0.31), (EVENING PRIMROSE OIL:Blood cholesterol abnormal:0.15), (FAMOTIDINE\IBUPROFEN:Blood cholesterol abnormal:0.16), (FENOTEROL HYDROBROMIDE\IPRATROPIUM BROMIDE:Blood cholesterol abnormal:0.52), (FERRIC POLYSACCHARIDE COMPLEX:Blood cholesterol abnormal:0.31), (FERROUS SULFATE\GLYCINE:Blood cholesterol abnormal:0.25), (FLUOCINOLONE ACETONIDE:Blood cholesterol abnormal:1.09), (FLURAZEPAM\FLURAZEPAM HYDROCHLORIDE:Blood cholesterol abnormal:0.13), (FOLATE SODIUM:Blood cholesterol abnormal:1.11), (FROVATRIPTAN SUCCINATE:Blood cholesterol abnormal:0.43), (FUSIDIC ACID:Blood cholesterol abnormal:1.17), (GINGER:Blood cholesterol abnormal:0.44), (GLIMEPIRIDE\ROSIGLITAZONE MALEATE:Blood cholesterol abnormal:0.48), (GLUTAMINE:Blood cholesterol abnormal:0.06), (GLUTATHIONE:Blood cholesterol abnormal:0.01), (GLYCOPYRROLATE\INDACATEROL MALEATE:Blood cholesterol abnormal:0.74), (HORSE CHESTNUT:Blood cholesterol abnormal:0.37), (ILOPROST:Blood cholesterol abnormal:1.33), (IMIGLUCERASE:Blood cholesterol abnormal:0.33), (INSULIN PORK\INSULIN PURIFIED PORK:Blood cholesterol abnormal:0.59), (INTERFERON:Blood cholesterol abnormal:1.09), (INTERFERON ALFA-2A:Blood cholesterol abnormal:0.81), (IRON SUCROSE:Blood cholesterol abnormal:0.48), (IXAZOMIB:Blood cholesterol abnormal:0.69), (LANREOTIDE:Blood cholesterol abnormal:0.33), (LAPATINIB DITOSYLATE:Blood cholesterol abnormal:0.54), (LENVATINIB:Blood cholesterol abnormal:0.07), (LEVALBUTEROL:Blood cholesterol abnormal:0.58), (LEVOLEUCOVORIN CALCIUM:Blood cholesterol abnormal:0.04), (LEVOMILNACIPRAN HYDROCHLORIDE:Blood cholesterol abnormal:0.54), (LORCASERIN HYDROCHLORIDE:Blood cholesterol abnormal:0.07), (LUTETIUM LU-177:Blood cholesterol abnormal:0.56), (MEGESTROL:Blood cholesterol abnormal:0.26), (MEPOLIZUMAB:Blood cholesterol abnormal:1.15), (METHENAMINE MANDELATE:Blood cholesterol abnormal:0.48), (METRELEPTIN:Blood cholesterol abnormal:0.66), (MOEXIPRIL HYDROCHLORIDE:Blood cholesterol abnormal:0.69), (MOXIFLOXACIN:Blood cholesterol abnormal:1.03), (MOXONIDINE:Blood cholesterol abnormal:0.54), (NADROPARIN CALCIUM:Blood cholesterol abnormal:0.51), (NALOXONE HYDROCHLORIDE\OXYCODONE HYDROCHLORIDE:Blood cholesterol abnormal:1.94), (NALOXONE HYDROCHLORIDE\TILIDINE HYDROCHLORIDE:Blood cholesterol abnormal:0.9), (NALTREXONE:Blood cholesterol abnormal:1), (NAPROXEN SODIUM\SUMATRIPTAN SUCCINATE:Blood cholesterol abnormal:0.2), (NARATRIPTAN HYDROCHLORIDE:Blood cholesterol abnormal:0.62), (NEOMYCIN:Blood cholesterol abnormal:0.17), (NISOLDIPINE:Blood cholesterol abnormal:0.43), (NORETHINDRONE:Blood cholesterol abnormal:0.97), (OLOPATADINE:Blood cholesterol abnormal:0.3), (OMEPRAZOLE SODIUM:Blood cholesterol abnormal:0.99), (OSELTAMIVIR PHOSPHATE:Blood cholesterol abnormal:0.71), (OXAPROZIN:Blood cholesterol abnormal:0.92), (OXYMORPHONE:Blood cholesterol abnormal:0.33), (PALONOSETRON HYDROCHLORIDE:Blood cholesterol abnormal:0.26), (PAPAYA:Blood cholesterol abnormal:0.14), (PARATHYROID HORMONE:Blood cholesterol abnormal:0.5), (PASIREOTIDE:Blood cholesterol abnormal:0.7), (PEGINTERFERON BETA-1A:Blood cholesterol abnormal:1.58), (PEMETREXED:Blood cholesterol abnormal:0.2), (PENICILLIN V POTASSIUM:Blood cholesterol abnormal:0.21), (PERTUZUMAB:Blood cholesterol abnormal:0.11), (PHENPROCOUMON:Blood cholesterol abnormal:0.8), (PINAVERIUM BROMIDE:Blood cholesterol abnormal:0.03), (PINDOLOL:Blood cholesterol abnormal:0.18), (PIRFENIDONE:Blood cholesterol abnormal:0.48), (POLYETHYLENE GLYCOL 3350\POTASSIUM CHLORIDE\SODIUM BICARBONATE\SODIUM CHLORIDE\SODIUM SULFATE ANHYDROUS:Blood cholesterol abnormal:0.56), (PRAMOXINE HYDROCHLORIDE:Blood cholesterol abnormal:0.17), (PREDNISOLONE SODIUM PHOSPHATE:Blood cholesterol abnormal:0.06), (PREDNISOLONE\PREDNISOLONE ACETATE\PREDNISOLONE SODIUM SUCCINATE:Blood cholesterol abnormal:0.4), (PROTRIPTYLINE HYDROCHLORIDE:Blood cholesterol abnormal:1.26), (QUINACRINE:Blood cholesterol abnormal:0.35), (RADIATION THERAPY:Blood cholesterol abnormal:0.73), (RIOCIGUAT:Blood cholesterol abnormal:0.55), (RUCAPARIB CAMSYLATE:Blood cholesterol abnormal:0.05), (SELEGILINE:Blood cholesterol abnormal:0.73), (SIBUTRAMINE HYDROCHLORIDE:Blood cholesterol abnormal:0.61), (SOFOSBUVIR\VELPATASVIR:Blood cholesterol abnormal:0.75), (TAFLUPROST:Blood cholesterol abnormal:0.42), (TEDUGLUTIDE\WATER:Blood cholesterol abnormal:0.44), (TELOTRISTAT ETHYL:Blood cholesterol abnormal:0.58), (TESAMORELIN:Blood cholesterol abnormal:0.36), (TETRACYCLINE:Blood cholesterol abnormal:1.79), (THALIDOMIDE:Blood cholesterol abnormal:1.41), (TIMOLOL\TRAVOPROST:Blood cholesterol abnormal:0.2), (TINZAPARIN:Blood cholesterol abnormal:0.55), (TRIMIPRAMINE MALEATE:Blood cholesterol abnormal:0.5), (TRIPTORELIN PAMOATE:Blood cholesterol abnormal:0.62), (VANCOMYCIN HYDROCHLORIDE:Blood cholesterol abnormal:0.18), (VEMURAFENIB:Blood cholesterol abnormal:0.63), (VINORELBINE TARTRATE:Blood cholesterol abnormal:0.04), (VORAPAXAR SULFATE:Blood cholesterol abnormal:0.04), (ZILEUTON:Blood cholesterol abnormal:0.87), (AMPHETAMINE ASPARTATE\AMPHETAMINE SULFATE\DEXTROAMPHETAMINE SACCHARATE\DEXTROAMPHETAMINE SULFATE:Blood cholesterol decreased:0.24),(ARMODAFINIL:Blood cholesterol decreased:0.11), (BENAZEPRIL HYDROCHLORIDE:Blood cholesterol decreased:0.2), (BISOPROLOL FUMARATE:Blood cholesterol decreased:0.55), (CALCITRIOL:Blood cholesterol decreased:0.23), (CALCIUM ACETATE:Blood cholesterol decreased:0.01), (CANDESARTAN:Blood cholesterol decreased:0.47), (CANDESARTAN CILEXETIL:Blood cholesterol decreased:0.42), (CLINDAMYCIN\CLINDAMYCIN PHOSPHATE:Blood cholesterol decreased:0.15), (CLOTRIMAZOLE:Blood cholesterol decreased:0.05), (COLCHICINE:Blood cholesterol decreased:0.08), (DALFAMPRIDINE:Blood cholesterol decreased:0.6), (DASATINIB:Blood cholesterol decreased:0.34), (DICLOFENAC:Blood cholesterol decreased:0.52), (DUTASTERIDE:Blood cholesterol decreased:0.38), (EMPAGLIFLOZIN\LINAGLIPTIN:Blood cholesterol decreased:0.01), (ENOXAPARIN:Blood cholesterol decreased:0.16), (ESTROGENS, CONJUGATED:Blood cholesterol decreased:0.24), (ESZOPICLONE:Blood cholesterol decreased:0.07), (FESOTERODINE FUMARATE:Blood cholesterol decreased:0.15), (GLICLAZIDE:Blood cholesterol decreased:0.01), (HYDROCHLOROTHIAZIDE\LOSARTAN POTASSIUM:Blood cholesterol decreased:0.14), (HYDROMORPHONE HYDROCHLORIDE:Blood cholesterol decreased:0.22), (HYDROXYZINE PAMOATE:Blood cholesterol decreased:0.07), (IBRUTINIB:Blood cholesterol decreased:0.8), (LITHIUM CARBONATE:Blood cholesterol decreased:0.14), (METRONIDAZOLE:Blood cholesterol decreased:0.67), (NEBIVOLOL:Blood cholesterol decreased:0.1), (PANTOPRAZOLE MAGNESIUM:Blood cholesterol decreased:0.4), (PROMETHAZINE\PROMETHAZINE HYDROCHLORIDE:Blood cholesterol decreased:0.16), (RISEDRONATE SODIUM:Blood cholesterol decreased:0.06), (SECUKINUMAB:Blood cholesterol decreased:0.24), (SODIUM BICARBONATE:Blood cholesterol decreased:0.05), (TERIFLUNOMIDE:Blood cholesterol decreased:0.68), (VARENICLINE TARTRATE:Blood cholesterol decreased:0.06), (ZOLEDRONIC ACID:Blood cholesterol decreased:0.08), (.ALPHA.-TOCOPHEROL ACETATE, D-\ASCORBIC ACID\CALCIUM PANTOTHENATE\FOLIC ACID\MANGANESE\NIACINAMIDE\PYRIDOXINE\RIBOFLAVIN\THIAMINE\VITAMIN A\ZINC OXIDE:Blood cholesterol increased:0.21), (. GAMMA.-AMINOBUTYRIC ACID:Blood cholesterol increased:0.08), (ACAMPROSATE:Blood cholesterol increased:0.02), (ACETAMINOPHEN\CAFFEINE\CARISOPRODOL\DICLOFENAC:Blood cholesterol increased:0.66), (ACETAMINOPHEN\CAFFEINE\OPIUM:Blood cholesterol increased:0.17), (ACETAMINOPHEN\CHLORPHENIRAMINE MALEATE\PHENYLEPHRINE HYDROCHLORIDE OR CETIRIZINE HYDROCHLORIDE OR CHLORPHENIRAMINE MALEATE OR CLEMASTINE FUMARATE OR DIPHENHYDRAMINE HYDROCHLORIDE OR FEXOFENADINE HYDROCHLORIDE OR LORATADINE:Blood cholesterol increased:0.05), (ACETAMINOPHEN\CHLORPHENIRAMINE MALEATE\PSEUDOEPHEDRINE HYDROCHLORIDE:Blood cholesterol increased:0.58), (ACETAMINOPHEN\METHOCARBAMOL:Blood cholesterol increased:1.28), (ACETYLCARNITINE HYDROCHLORIDE:Blood cholesterol increased:0.89), (ACRIVASTINE\PSEUDOEPHEDRINE HYDROCHLORIDE:Blood cholesterol increased:0.39), (ADAPALENE\BENZOYL PEROXIDE:Blood cholesterol increased:0.16), (ALDESLEUKIN:Blood cholesterol increased:0.1), (ALFALFA:Blood cholesterol increased:0.73), (ALISKIREN\HYDROCHLOROTHIAZIDE:Blood cholesterol increased:0.17), (ALLANTOIN\BENZOCAINE\CAMPHOR (NATURAL)\DIMETHICONE\MENTHOL:Blood cholesterol increased:0.3), (ALPHA-TOCOPHEROL\ASCORBIC ACID\CHOLECALCIFEROL\COPPER\CYANOCOBALAMIN\FOLIC ACID\LUTEIN\OMEGA-3 FATTY ACIDS\PYRIDOXINE HYDROCHLORIDE\TAURINE\VITAMIN A PALMITATE\ZEAXANTHIN\ZINC PICOLINATE:Blood cholesterol increased:0.06), (ALUMINUM CHLORIDE:Blood cholesterol increased:0.36), (AMIKACIN:Blood cholesterol increased:0.27), (AMILORIDE HYDROCHLORIDE\FUROSEMIDE:Blood cholesterol increased:0.74), (AMITRIPTYLINE\PERPHENAZINE:Blood cholesterol increased:0.22), (AMOXICILLIN\CLARITHROMYCIN\ESOMEPRAZOLE:Blood cholesterol increased:l), (AMPHETAMINE SULFATE\DEXTROAMPHETAMINE:Blood cholesterol increased:0.08), (AMPHETAMINE\DEXTROAMPHETAMINE:Blood cholesterol increased:0), (AMPICILLIN SODIUM\SULBACTAM SODIUM:Blood cholesterol increased:0.41), (ARNICA MONTANA:Blood cholesterol increased:0.03), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Blood cholesterol increased:0.95), (ASCORBIC ACID\FERROUS SULFATE\FOLIC ACID\PYRIDOXINE HYDROCHLORIDE\RIBOFLAVIN\THIAMINE MONONITRATE:Blood cholesterol increased:0.34), (ASPIRIN\BUTALBITAL\CAFFEINE\CODEINE PHOSPHATE:Blood cholesterol increased:0.57), (ASPIRIN\CAFFEINE\SALICYLAMIDE:Blood cholesterol increased:0.97), (ASPIRIN\CLOPIDOGREL:Blood cholesterol increased:0.2), (ATOMOXETINE:Blood cholesterol increased:0.46), (ATROPINE SULFATE\DIPHENOXYLATE:Blood cholesterol increased:0.09), (AZITHROMYCIN MONOHYDRATE:Blood cholesterol increased:0.87), (AZTREONAM:Blood cholesterol increased:0.06), (BACITRACIN ZINC:Blood cholesterol increased:0.82), (BACITRACIN\NEOMYCIN SULFATE\POLYMYXIN B SULFATE:Blood cholesterol increased:0.29), (BACITRACIN\NEOMYCIN\POLYMYXIN B:Blood cholesterol increased:0.01), (BECLOMETHASONE DIPROPIONATE\FORMOTEROL:Blood cholesterol increased:0.17), (BENSERAZIDE HYDROCHLORIDE\LEVODOPA:Blood cholesterol increased:0.05), (BEPOTASTINE\BEPOTASTINE BESILATE:Blood cholesterol increased:0.08), (BETAMETHASONE DIPROPIONATE\CALCIPOTRIENE HYDRATE:Blood cholesterol increased:0.26), (BICARBONATE ION:Blood cholesterol increased:0.02), (BIFIDOBACTERIUM LONGUM\LACTOBACILLUS ACIDOPHILUS:Blood cholesterol increased:0.28), (BIPERIDEN HYDROCHLORIDE:Blood cholesterol increased:0.67), (BISMUTH SUBSALICYLATE\GLYCERIN\PROPYLENE GLYCOL:Blood cholesterol increased:0.19), (BLINATUMOMAB:Blood cholesterol increased:0.65), (BOCEPREVIR:Blood cholesterol increased:0.75), (BORIC ACID:Blood cholesterol increased:0.22), (BUPRENORPHINE\NALOXONE:Blood cholesterol increased:0.93), (BUPROPION HYDROBROMIDE:Blood cholesterol increased:0.57), (BUTABARBITAL:Blood cholesterol increased:0.14), (CABOZANTINIB:Blood cholesterol increased:0.28), (CALCIUM BICARBONATE:Blood cholesterol increased:0.17), (CALCIUM CARBIMIDE:Blood cholesterol increased:0.11), (CALCIUM CHLORIDE\DEXTROSE MONOHYDRATE\MAGNESIUM CHLORIDE\SODIUM CHLORIDE\SODIUM LACTATE:Blood cholesterol increased:0.04), (CALCIUM PANTOTHENATE\CUPRIC SULFATE\CYANOCOBALAMIN\FERROUS ASPARTO GLYCINATE\FERROUS FUMARATE\FOLIC ACID\MAGNESIUM SULFATE\NIACINAMIDE\PYRIDOXINE HYDROCHLORIDE\RIBOFLAVIN\SODIUM ASCORBATE\THIAMINE MONONITRATE\ZINC SULFATE:Blood cholesterol increased:0.02), (CALCIUM PANTOTHENATE\CYANOCOBALAMIN\NIACINAMIDE\PYRIDOXINE HYDROCHLORIDE\RIBOFLAVIN\THIAMINE HYDROCHLORIDE:Blood cholesterol increased:0.68), (CALCIUM\MAGNESIUM\VITAMIN D\ZINC:Blood cholesterol increased:0.04), (CAMPHOR (SYNTHETIC)\LEVOMENTHOL\MENTHYL SALICYLATE OR CAMPHOR (SYNTHETIC)\MENTHOL\MENTHYL SALICYLATE OR MENTHOL\MENTHYL SALICYLATE:Blood cholesterol increased:0.17), (CAPSICUM:Blood cholesterol increased:0.6), (CARBIMAZOLE:Blood cholesterol increased:0.87), (CEFAZOLIN:Blood cholesterol increased:1.38), (CEFEPIME\CEFEPIME HYDROCHLORIDE:Blood cholesterol increased:0.37), (CEFOTAXIME SODIUM:Blood cholesterol increased:0.49), (CEFPROZIL:Blood cholesterol increased:0.55), (CERITINIB:Blood cholesterol increased:0.69), (CHLORDIAZEPOXIDE\CLIDINIUM:Blood cholesterol increased:0.36), (CHLORMADINONE ACETATE\ETHINYL ESTRADIOL:Blood cholesterol increased:0.13), (CHLOROQUINE PHOSPHATE:Blood cholesterol increased:0.39), (CHLORPROTHIXENE:Blood cholesterol increased:0.29), (CHOLECALCIFEROL\GENISTEIN\ZINC GLYCINATE:Blood cholesterol increased:0.46), (CHOLINE\TRAZODONE HYDROCHLORIDE:Blood cholesterol increased:0.28), (CHONDROITIN SULFATE (CHICKEN):Blood cholesterol increased:0.09), (CILASTATIN SODIUM\IMIPENEM:Blood cholesterol increased:1.48), (CIPROFLOXACIN\CIPROFLOXACIN HYDROCHLORIDE:Blood cholesterol increased:0.3), (CIPROFLOXACIN\DEXAMETHASONE:Blood cholesterol increased:0.04), (COCOA BUTTER\PETROLATUM\PHENYLEPHRINE HYDROCHLORIDE\PHENYLEPHRINE HYDROCHLORIDE OR MINERAL OIL:Blood cholesterol increased:0.06), (CODEINE PHOSPHATE\PROMETHAZINE:Blood cholesterol increased:0.51), (COLISTIMETHATE SODIUM:Blood cholesterol increased:0), (CRANBERRY CONCENTRATE:Blood cholesterol increased:0.73), (CREATINE MONOHYDRATE:Blood cholesterol increased:0.46), (CREATININE:Blood cholesterol increased:0.1), (CROFELEMER:Blood cholesterol increased:0.35), (CYANOCOBALAMIN\DICLOFENAC SODIUM\PYRIDOXINE HYDROCHLORIDE\THIAMINE MONONITRATE:Blood cholesterol increased:0.76), (CYCLOPENTOLATE:Blood cholesterol increased:0.41), (CYPROHEPTADINE HYDROCHLORIDE:Blood cholesterol increased:1.07), (DACLATASVIR:Blood cholesterol increased:0.22), (DACLIZUMAB:Blood cholesterol increased:2.41), (DANTROLENE SODIUM:Blood cholesterol increased:0.97), (DEFERIPRONE:Blood cholesterol increased:0.61), (DEGARELIX:Blood cholesterol increased:0.07), (DESOGESTREL\ETHINYL ESTRADIOL:Blood cholesterol increased:0.11), (DEXAMETHASONE\NEOMYCIN\POLYMYXIN B SULFATE:Blood cholesterol increased:0.16), (DEXTROMETHORPHAN\DEXTROMETHORPHAN HYDROBROMIDE\GUAIFENESIN:Blood cholesterol increased:0.04), (DICHLORPHENAMIDE:Blood cholesterol increased:0.43), (DICLOFENAC DIETHYLAMINE:Blood cholesterol increased:0.36), (DIETARY SUPPLEMENT\FLAVOCOXID:Blood cholesterol increased:0.31), (DIETARY SUPPLEMENT\HERBALS\MILK THISTLE:Blood cholesterol increased:0.65), (DIETARY SUPPLEMENT\RESVERATROL:Blood cholesterol increased:0.43), (DIHYDROERGOTAMINE MESYLATE:Blood cholesterol increased:0.55), (DIMETHYL SULFOXIDE:Blood cholesterol increased:0.81), (DOCONEXENT\ICOSAPENT\FISH OIL\OMEGA-3 FATTY ACIDS:Blood cholesterol increased:0.1), (DOLASETRON MESYLATE:Blood cholesterol increased:1.51), (DOXYCYCLINE HYDROCHLORIDE:Blood cholesterol increased:0.35), (DOXYLAMINE SUCCINATE\PYRIDOXINE HYDROCHLORIDE:Blood cholesterol increased:0.11), (DROSPIRENONE\ESTRADIOL:Blood cholesterol increased:0.26), (ELBASVIR\GRAZOPREVIR:Blood cholesterol increased:0.23), (ELOTUZUMAB:Blood cholesterol increased:1), (EMTRICITABINE\RILPIVIRINE HYDROCHLORIDE\TENOFOVIR DISOPROXIL FUMARATE:Blood cholesterol increased:0.77), (EMTRICITABINE\RILPIVIRINE\TENOFOVIR DISOPROXIL FUMARATE:Blood cholesterol increased:0.1), (EMTRICITABINE\TENOFOVIR ALAFENAMIDE FUMARATE:Blood cholesterol increased:0.1), (ENALAPRIL MALEATE\LERCANIDIPINE HYDROCHLORIDE:Blood cholesterol increased:0.25), (EPHEDRINE HYDROCHLORIDE\GUAIFENESIN:Blood cholesterol increased:0.6), (EPIRUBICIN HYDROCHLORIDE:Blood cholesterol increased:0.12), (ERIBULIN:Blood cholesterol increased:0.47), (ERTAPENEM SODIUM:Blood cholesterol increased:0.53), (ESTRADIOL ACETATE:Blood cholesterol increased:1.2), (ESTRADIOL BENZOATE:Blood cholesterol increased:1.04), (ESTRADIOL\LEVONORGESTREL:Blood cholesterol increased:0.69), (ESTROGENS\METHYLTESTOSTERONE:Blood cholesterol increased:0.68), (ESTRONE:Blood cholesterol increased:0), (ETHINYL ESTRADIOL\FERROUS FUMARATE\NORETHINDRONE:Blood cholesterol increased:0.41), (ETHOSUXIMIDE:Blood cholesterol increased:0.45), (FENOPROFEN CALCIUM:Blood cholesterol increased:0.5), (FERRIC HYDROXIDE:Blood cholesterol increased:0.47), (FERRIC POLYSACCHARIDE COMPLEX:Blood cholesterol increased:1.14), (FERRITIN:Blood cholesterol increased:0.09), (FERROUS SULFATE\FOLIC ACID:Blood cholesterol increased:0.14), (FEXOFENADINE HYDROCHLORIDE\PSEUDOEPHEDRINE HYDROCHLORIDE:Blood cholesterol increased:0.83), (FILGRASTIM-SNDZ:Blood cholesterol increased:0.42), (FLUINDIONE:Blood cholesterol increased:0.87), (FLUPENTIXOL DIHYDROCHLORIDE\MELITRACEN HYDROCHLORIDE:Blood cholesterol increased:0.05), (FLURBIPROFEN:Blood cholesterol increased:0.41), (FLUTAMIDE:Blood cholesterol increased:0.2), (GENISTEIN:Blood cholesterol increased:0.57), (GENTIAN VIOLET:Blood cholesterol increased:0.09), (GIMERACIL\OTERACIL\TEGAFUR:Blood cholesterol increased:0.32), (GLATIRAMER:Blood cholesterol increased:0.52), (GLUCAGON HYDROCHLORIDE:Blood cholesterol increased:0.04), (GLUCONATE:Blood cholesterol increased:0.38), (GLUCOSAMINE HYDROCHLORIDE:Blood cholesterol increased:0.24), (GLYCOPYRROLATE\INDACATEROL MALEATE:Blood cholesterol increased:1.45), (GREEN TEA LEAF:Blood cholesterol increased:0.42), (GUAIFENESIN\PHENYLEPHRINE HYDROCHLORIDE:Blood cholesterol increased:0.16), (HEMP:Blood cholesterol increased:0.15), (HEPARIN CALCIUM:Blood cholesterol increased:0.13), (HERBALS\HOMEOPATHICS\GOLDENSEAL:Blood cholesterol increased:0.32), (HERBALS\MINERALS\VITAMINS:Blood cholesterol increased:0.86), (HERBALS\VITIS VINIFERA SEED:Blood cholesterol increased:0.52), (HIGH DENSITY POLYETHYLENE:Blood cholesterol increased:0.33), (HYDROCHLORIC ACID:Blood cholesterol increased:0.7), (HYDROCORTISONE\NEOMYCIN SULFATE\POLYMYXIN B SULFATE:Blood cholesterol increased:0.04), (HYDROQUINONE:Blood cholesterol increased:0.7), (HYPERICUM PERFORATUM:Blood cholesterol increased:0.33), (ILOPROST:Blood cholesterol increased:1.47), (IMIPENEM:Blood cholesterol increased:0.1), (IMMUNE GLOBULIN NOS:Blood cholesterol increased:0.38), (INCOBOTULINUMTOXINA:Blood cholesterol increased:0.12), (INSULIN PORK\INSULIN PURIFIED PORK:Blood cholesterol increased:1.45), (INTERFERON:Blood cholesterol increased:2.4), (INTERFERON ALFA-2A:Blood cholesterol increased:1.42), (INTERFERON ALFA-2B:Blood cholesterol increased:1.29), (INTERFERON GAMMA-1B:Blood cholesterol increased:0.89), (IRINOTECAN HYDROCHLORIDE:Blood cholesterol increased:1.28), (IRON DEXTRAN:Blood cholesterol increased:0.16), (ISOFLAVONES SOY:Blood cholesterol increased:0.14), (IXAZOMIB CITRATE:Blood cholesterol increased:0), (KERATIN:Blood cholesterol increased:0.09), (LACTOPEROXIDASE BOVINE\LYSOZYME HYDROCHLORIDE:Blood cholesterol increased:0.25), (LENVATINIB:Blood cholesterol increased:0.66), (LETROZOLE\RIBOCICLIB:Blood cholesterol increased:0.11), (LEVAMLODIPINE:Blood cholesterol increased:0.17), (LEVOLEUCOVORIN CALCIUM:Blood cholesterol increased:0.14), (LEVOMEFOLATE CALCIUM\METHYLCOBALAMIN\PYRIDOXAL 5-PHOSPHATE:Blood cholesterol increased:0.23), (LEVOMEFOLIC ACID\METHYLCOBALAMIN\TURMERIC:Blood cholesterol increased:0.6), (LEVORPHANOL TARTRATE:Blood cholesterol increased:0.05), (LEVOTHYROXINE SODIUM\POTASSIUM IODIDE:Blood cholesterol increased:0.11), (LINSIDOMINE:Blood cholesterol increased:0.25), (LOMUSTINE:Blood cholesterol increased:0.07), (LUTETIUM LU-177:Blood cholesterol increased:1.24), (MAGNESIUM SULFATE\POTASSIUM SULFATE\SODIUM SULFATE:Blood cholesterol increased:0.46), (MAPROTILINE HYDROCHLORIDE:Blood cholesterol increased:0.09), (MATRICARIA RECUTITA:Blood cholesterol increased:0.16), (MELPERONE:Blood cholesterol increased:0.17), (MESNA:Blood cholesterol increased:0.66), (MESTRANOL\NORETHINDRONE:Blood cholesterol increased:0.33), (METFORMIN HYDROCHLORIDE\SAXAGLIPTIN:Blood cholesterol increased:1.16), (METHENAMINE MANDELATE:Blood cholesterol increased:1.4), (METHOXY POLYETHYLENE GLYCOL-EPOETIN BETA:Blood cholesterol increased:0.24), (METRELEPTIN:Blood cholesterol increased:1.33), (MIGLUSTAT:Blood cholesterol increased:0), (NADH:Blood cholesterol increased:0), (NALOXONE HYDROCHLORIDE:Blood cholesterol increased:0.45), (NALOXONE HYDROCHLORIDE\PENTAZOCINE HYDROCHLORIDE:Blood cholesterol increased:0.16), (NIACINAMIDE\PYRIDOXINE\RIBOFLAVIN\THIAMINE:Blood cholesterol increased:0.06), (NICARDIPINE:Blood cholesterol increased:0.9), (NOREPINEPHRINE:Blood cholesterol increased:0.29), (OBETICHOLIC ACID:Blood cholesterol increased:0.08), (OMEGA-6 FATTY ACIDS:Blood cholesterol increased:0.06), (OPIPRAMOL:Blood cholesterol increased:1.01), (OXYQUINOLINE SULFATE\SODIUM LAURYL SULFATE:Blood cholesterol increased:0.09), (PARSLEY:Blood cholesterol increased:0.46), (PEMETREXED DISODIUM:Blood cholesterol increased:1.56), (PENICILLAMINE:Blood cholesterol increased:0.13), (PENTOSTATIN:Blood cholesterol increased:0.44), (PEPPERMINT:Blood cholesterol increased:0.27), (PHENYLEPHRINE HYDROCHLORIDE\PROMETHAZINE HYDROCHLORIDE:Blood cholesterol increased:0.39), (PHENYLEPHRINE HYDROCHLORIDE\WITCH HAZEL:Blood cholesterol increased:0.26), (PIZOTYLINE:Blood cholesterol increased:0.13), (PLERIXAFOR:Blood cholesterol increased:0.07), (PODOFILOX:Blood cholesterol increased:0.69), (POLYVINYL ALCOHOL\POVIDONE:Blood cholesterol increased:1.03), (POTASSIUM BITARTRATE:Blood cholesterol increased:0.16), (POTASSIUM PHOSPHATE, MONOBASIC:Blood cholesterol increased:0.39), (PREDNISOLONE ACETATE\SULFACETAMIDE SODIUM:Blood cholesterol increased:0.64), (PREDNISOLONE\PREDNISOLONE ACETATE\PREDNISOLONE SODIUM SUCCINATE:Blood cholesterol increased:0.81), (PREGNENOLONE:Blood cholesterol increased:1.26), (PROCAINE:Blood cholesterol increased:0.38), (PRUCALOPRIDE SUCCINATE:Blood cholesterol increased:0.53), (PSORALEN:Blood cholesterol increased:0.31), (QUINACRINE:Blood cholesterol increased:1.34), (QUINAGOLIDE HYDROCHLORIDE:Blood cholesterol increased:0.2), (QUINIDINE GLUCONATE:Blood cholesterol increased:0.77), (RAMUCIRUMAB:Blood cholesterol increased:0.05), (RESLIZUMAB:Blood cholesterol increased:0.25), (RILMENIDINE PHOSPHATE:Blood cholesterol increased:0.44), (RUCAPARIB CAMSYLATE:Blood cholesterol increased:0.7), (RUFINAMIDE:Blood cholesterol increased:0.16), (RUPATADINE FUMARATE:Blood cholesterol increased:0.01), (SEVELAMER:Blood cholesterol increased:0.36), (SODIUM FLUORIDE\VITAMINS:Blood cholesterol increased:0.48), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Blood cholesterol increased:0.14), (SOFOSBUVIR\VELPATASVIR:Blood cholesterol increased:1.32), (SUFENTANIL CITRATE:Blood cholesterol increased:0.16), (SULTOPRIDE HYDROCHLORIDE:Blood cholesterol increased:0.22), (TALIGLUCERASE ALFA:Blood cholesterol increased:0.1), (TEDUGLUTIDE:Blood cholesterol increased:0.53), (TEGASEROD:Blood cholesterol increased:0.14), (TEGASEROD MALEATE:Blood cholesterol increased:0.67), (TENOXICAM:Blood cholesterol increased:0.19), (THIOTHIXENE:Blood cholesterol increased:0.49), (THYROTROPIN ALFA:Blood cholesterol increased:0.08), (TICLOPIDINE:Blood cholesterol increased:0.27), (TIGECYCLINE:Blood cholesterol increased:0.1), (TILIDINE:Blood cholesterol increased:0.31), (TOLMETIN SODIUM:Blood cholesterol increased:0.09), (TREPROSTINIL DIOLAMINE:Blood cholesterol increased:0.47), (TRIENTINE HYDROCHLORIDE:Blood cholesterol increased:0), (TRIMIPRAMINE MALEATE:Blood cholesterol increased:1.15), (TYROSINE:Blood cholesterol increased:0.14), (UNDECYLENIC ACID:Blood cholesterol increased:0.06), (VITAMIN A PALMITATE:Blood cholesterol increased:0.47), (VORAPAXAR SULFATE:Blood cholesterol increased:0.55), (XYLITOL:Blood cholesterol increased:0), (ZIDOVUDINE:Blood cholesterol increased:0.03), (ZUCLOPENTHIXOL:Blood cholesterol increased:0.11), (.ALPHA.1-PROTEINASE INHIBITOR HUMAN:Blood disorder:0.6), (ACARBOSE:Blood disorder:0.68), (ACETAMINOPHEN\OXYCODONE HYDROCHLORIDE\OXYCODONE TEREPHTHALATE:Blood disorder:0.1), (ALCOHOL:Blood disorder:0.24), (ALEMTUZUMAB:Blood disorder:0.04), (ALENDRONIC ACID:Blood disorder:0.24), (ALFACALCIDOL:Blood disorder:0.27), (AMANTADINE:Blood disorder:0.42), (AMLODIPINE BESYLATE\HYDROCHLOROTHIAZIDE\OLMESARTAN MEDOXOMIL:Blood disorder:0.03), (AMMONIUM LACTATE:Blood disorder:0.15), (BENAZEPRIL\BENAZEPRIL HYDROCHLORIDE:Blood disorder:0.56), (BETAMETHASONE DIPROPIONATE\CALCIPOTRIENE\CALCIPOTRIENE HYDRATE:Blood disorder:0.16), (BICALUTAMIDE:Blood disorder:0.49), (BIFIDOBACTERIUM LONGUM SUBSP. INFANTIS:Blood disorder:0.13), (BUPRENORPHINE HYDROCHLORIDE:Blood disorder:0.03), (CANAKINUMAB:Blood disorder:0.26), (CEFTRIAXONE SODIUM:Blood disorder:0.03), (CETIRIZINE HYDROCHLORIDE\PSEUDOEPHEDRINE HYDROCHLORIDE:Blood disorder:0.2), (CILASTATIN SODIUM\IMIPENEM:Blood disorder:0.23), (CLARITHROMYCIN:Blood disorder:0.09), (CLINDAMYCIN\CLINDAMYCIN PHOSPHATE:Blood disorder:1.03), (CODEINE:Blood disorder:0.03), (CORTICOTROPIN:Blood disorder:0.36), (DABIGATRAN:Blood disorder:0.46), (DACLIZUMAB:Blood disorder:0.73), (DALFAMPRIDINE:Blood disorder:1), (DASATINIB:Blood disorder:1.43), (DEFLAZACORT:Blood disorder:0.09), (DESLORATADINE:Blood disorder:0.69), (DIMENHYDRINATE:Blood disorder:0.34), (DRONABINOL:Blood disorder:0.61), (EMPAGLIFLOZIN\LINAGLIPTIN:Blood disorder:1), (EMPAGLIFLOZIN\METFORMIN HYDROCHLORIDE:Blood disorder:0.16), (ESTROGENS, CONJUGATED\MEDROXYPROGESTERONE ACETATE:Blood disorder:0.26), (ETORICOXIB:Blood disorder:0.3), (FENOFIBRIC ACID:Blood disorder:0.29), (FINGOLIMOD HYDROCHLORIDE:Blood disorder:0.33), (FLUOCINONIDE:Blood disorder:0.05), (GLYCOPYRRONIUM\INDACATEROL:Blood disorder:0.89), (HYDROXYCHLOROQUINE DIPHOSPHATE:Blood disorder:0.14), (ILOPROST:Blood disorder:0.25), (IMATINIB MESYLATE:Blood disorder:1), (INDAPAMIDE\PERINDOPRIL:Blood disorder:0.6), (ITRACONAZOLE:Blood disorder:0.04), (LERCANIDIPINE:Blood disorder:1.05), (LIDOCAINE HYDROCHLORIDE:Blood disorder:0.72), (LISDEXAMFETAMINE DIMESYLATE:Blood disorder:0.82), (LITHIUM CARBONATE:Blood disorder:0.84), (LOMITAPIDE MESYLATE:Blood disorder:0.55), (MEGESTROL ACETATE:Blood disorder:0.13), (MEROPENEM:Blood disorder:0.13), (METHENAMINE HIPPURATE:Blood disorder:0.64), (METRONIDAZOLE\METRONIDAZOLE HYDROCHLORIDE:Blood disorder:0.31), (MINOCYCLINE\MINOCYCLINE HYDROCHLORIDE:Blood disorder:0.09), (NALOXONE HYDROCHLORIDE\OXYCODONE HYDROCHLORIDE:Blood disorder:0.68), (NATEGLINIDE:Blood disorder:0.6), (NINTEDANIB:Blood disorder:0.33), (PARATHYROID HORMONE:Blood disorder:0.01), (PATIROMER:Blood disorder:0.36), (PEGINTERFERON BETA-1A:Blood disorder:0.77), (PEMETREXED DISODIUM:Blood disorder:0.01), (PENTOSAN POLYSULFATE SODIUM:Blood disorder:0.01), (PIROXICAM:Blood disorder:0.11), (PONATINIB:Blood disorder:0.75), (PREDNISOLONE ACETATE:Blood disorder:0.26), (PROPAFENONE HYDROCHLORIDE:Blood disorder:0.44), (QUETIAPINE:Blood disorder:0.91), (RABEPRAZOLE:Blood disorder:0.9), (RISEDRONATE SODIUM:Blood disorder:0.78), (ROSIGLITAZONE MALEATE:Blood disorder:0.05), (SALMETEROL:Blood disorder:0.21), (SITAGLIPTIN:Blood disorder:0.07), (SUMATRIPTAN\SUMATRIPTAN SUCCINATE:Blood disorder:0.66), (TERAZOSIN HYDROCHLORIDE:Blood disorder:0.19), (TERBINAFINE HYDROCHLORIDE:Blood disorder:0.32), (TETRACYCLINE:Blood disorder:0.51), (TIOTROPIUM:Blood disorder:0.56), (TOBRAMYCIN:Blood disorder:0.08), (TOCILIZUMAB:Blood disorder:0.5), (UMECLIDINIUM BROMIDE:Blood disorder:0.12), (VILAZODONE HYDROCHLORIDE:Blood disorder:0.27), (VITAMIN A:Blood disorder:0.36), (ZAFIRLUKAST:Blood disorder:0.26), (ZILEUTON:Blood disorder:0.06), (AMPHETAMINE ASPARTATE\AMPHETAMINE SULFATE\DEXTROAMPHETAMINE SACCHARATE\DEXTROAMPHETAMINE SULFATE:Blood glucose:0.04), (ANASTROZOLE:Blood glucose:0.26), (BUDESONIDE\FORMOTEROL FUMARATE:Blood glucose:0.19), (BUPROPION:Blood glucose:0.09), (CALCIUM ACETATE:Blood glucose:0.63), (CANAGLIFLOZIN\METFORMIN HYDROCHLORIDE:Blood glucose:0.16), (CANDESARTAN CILEXETIL\HYDROCHLOROTHIAZIDE:Blood glucose:0.53), (CAPTOPRIL:Blood glucose:0.05), (CARVEDILOL PHOSPHATE:Blood glucose:0.3), (CEPHALEXIN:Blood glucose:0.44), (CHOLESTYRAMINE:Blood glucose:0.62), (CORTISONE\HYDROCORTISONE:Blood glucose:0.25), (CYCLOBENZAPRINE:Blood glucose:0.36), (DACLIZUMAB:Blood glucose:0.02), (DIVALPROEX SODIUM:Blood glucose:0.07), (DOXAZOSIN\DOXAZOSIN MESYLATE:Blood glucose:0.51), (DUTASTERIDE:Blood glucose:0.43), (EMPAGLIFLOZIN\METFORMIN HYDROCHLORIDE:Blood glucose:0.25), (EPLERENONE:Blood glucose:0.12), (EVOLOCUMAB:Blood glucose:1.2), (EZETIMIBE\SIMVASTATIN:Blood glucose:0.18), (FESOTERODINE FUMARATE:Blood glucose:0.08), (FLECAINIDE:Blood glucose:0.39), (FLUTICASONE:Blood glucose:0.32), (HYDROMORPHONE HYDROCHLORIDE:Blood glucose:0.11), (HYDROXYCHLOROQUINE SULFATE:Blood glucose:0.07), (IBRUTINIB:Blood glucose:1.14), (ILOPROST:Blood glucose:0.08), (INDAPAMIDE\PERINDOPRIL:Blood glucose:0.13), (LEDIPASVIR\SOFOSBUVIR:Blood glucose:0.02), (LERCANIDIPINE:Blood glucose:1.08), (LEVALBUTEROL HYDROCHLORIDE:Blood glucose:0.28), (METRONIDAZOLE\METRONIDAZOLE HYDROCHLORIDE:Blood glucose:0), (NALOXONE HYDROCHLORIDE\OXYCODONE HYDROCHLORIDE:Blood glucose:0.23), (NORTRIPTYLINE:Blood glucose:0.01), (OLANZAPINE:Blood glucose:0.07), (OXCARBAZEPINE:Blood glucose:0.05), (PANTOPRAZOLE MAGNESIUM:Blood glucose:0.58), (PAROXETINE\PAROXETINE HYDROCHLORIDE:Blood glucose:0.18), (PRAMIPEXOLE DIHYDROCHLORIDE:Blood glucose:0.28), (PRIMIDONE:Blood glucose:0.22), (RISEDRONATE SODIUM:Blood glucose:0.09), (ROFLUMILAST:Blood glucose:0.12), (SACUBITRIL\VALSARTAN:Blood glucose:0.67), (SODIUM OXYBATE:Blood glucose:0.04), (SOTALOL:Blood glucose:0.58), (TERIFLUNOMIDE:Blood glucose:0.6), (THALIDOMIDE:Blood glucose:0.01), (TIZANIDINE HYDROCHLORIDE:Blood glucose:0.21), (TOCILIZUMAB:Blood glucose:0.05), (TRIAMTERENE:Blood glucose:0.06), (VALGANCICLOVIR HYDROCHLORIDE:Blood glucose:0.2), (ACETAMINOPHEN\OXYCODONE HYDROCHLORIDE\OXYCODONE TEREPHTHALATE:Blood glucose abnormal:0.19), (ACETAMINOPHEN\TRAMADOL:Blood glucose abnormal:0.07), (ACETAZOLAMIDE:Blood glucose abnormal:0.54), (AGALSIDASE BETA:Blood glucose abnormal:0.04), (ALISKIREN HEMIFUMARATE:Blood glucose abnormal:0.46), (ALUMINUM HYDROXIDE\DIMETHICONE\MAGNESIUM HYDROXIDE:Blood glucose abnormal:0.06), (BETAMETHASONE DIPROPIONATE\CALCIPOTRIENE\CALCIPOTRIENE HYDRATE:Blood glucose abnormal:0.03), (BIFIDOBACTERIUM LONGUM SUBSP. INFANTIS:Blood glucose abnormal:0.27), (BORTEZOMIB:Blood glucose abnormal:0.54), (BRINZOLAMIDE:Blood glucose abnormal:0.4), (BROMAZEPAM:Blood glucose abnormal:0.19), (CALCIUM\MAGNESIUM\ZINC:Blood glucose abnormal:0.5), (CANAKINUMAB:Blood glucose abnormal:0.06), (CANDESARTAN CILEXETIL\HYDROCHLOROTHIAZIDE:Blood glucose abnormal:1.08), (CARVEDILOL PHOSPHATE:Blood glucose abnormal:1.34), (CERTOLIZUMAB PEGOL:Blood glucose abnormal:1.12), (CETIRIZINE HYDROCHLORIDE\PSEUDOEPHEDRINE HYDROCHLORIDE:Blood glucose abnormal:0.2), (CIMETIDINE:Blood glucose abnormal:0.41), (CLOZAPINE:Blood glucose abnormal:0.55), (CODEINE:Blood glucose abnormal:0.14), (DABIGATRAN:Blood glucose abnormal:0.28), (DACLIZUMAB:Blood glucose abnormal:0.94), (DASATINIB:Blood glucose abnormal:1.08), (DOXEPIN HYDROCHLORIDE:Blood glucose abnormal:0.29), (DRONABINOL:Blood glucose abnormal:0.4), (ELETRIPTAN HYDROBROMIDE:Blood glucose abnormal:1.04), (ERYTHROMYCIN:Blood glucose abnormal:0.2), (ESOMEPRAZOLE MAGNESIUM\NAPROXEN:Blood glucose abnormal:0.1), (ESTROGENS, CONJUGATED\MEDROXYPROGESTERONE ACETATE:Blood glucose abnormal:0.36), (ETORICOXIB:Blood glucose abnormal:0.48), (FLUDROCORTISONE ACETATE:Blood glucose abnormal:0.22), (FLUOCINONIDE:Blood glucose abnormal:0.11), (FLUTICASONE:Blood glucose abnormal:0.95), (FLUTICASONE FUROATE\SALMETEROL XINAFOATE:Blood glucose abnormal:0.61), (FORMOTEROL:Blood glucose abnormal:0.03), (FORMOTEROL FUMARATE\MOMETASONE FUROATE:Blood glucose abnormal:0.71), (GLYCOPYRRONIUM:Blood glucose abnormal:0.26), (GLYCOPYRRONIUM\INDACATEROL:Blood glucose abnormal:0.75), (HERBALS\TURMERIC:Blood glucose abnormal:0.52), (HYDROMORPHONE:Blood glucose abnormal:0.81), (HYDROXYCHLOROQUINE DIPHOSPHATE:Blood glucose abnormal:0.07), (HYDROXYUREA:Blood glucose abnormal:0.05), (HYDROXYZINE PAMOATE:Blood glucose abnormal:1.31), (ILOPROST:Blood glucose abnormal:0.05), (IMATINIB MESYLATE:Blood glucose abnormal:0.25), (INDAPAMIDE\PERINDOPRIL:Blood glucose abnormal:0.46), (INDOMETHACIN:Blood glucose abnormal:0.41), (KETOROLAC\KETOROLAC TROMETHAMINE:Blood glucose abnormal:0.11), (LANREOTIDE ACETATE:Blood glucose abnormal:0.56), (LEDIPASVIR\SOFOSBUVIR:Blood glucose abnormal:0.53), (LERCANIDIPINE:Blood glucose abnormal:1.43), (LEUCOGEN:Blood glucose abnormal:0.05), (LIOTHYRONINE:Blood glucose abnormal:0.05), (LISDEXAMFETAMINE DIMESYLATE:Blood glucose abnormal:0.89), (LOMITAPIDE MESYLATE:Blood glucose abnormal:0.62), (MEPOLIZUMAB:Blood glucose abnormal:0.43), (METHIMAZOLE:Blood glucose abnormal:0.07), (MIDODRINE:Blood glucose abnormal:0.5), (MOXIFLOXACIN:Blood glucose abnormal:0.46), (MUPIROCIN\MUPIROCIN CALCIUM:Blood glucose abnormal:0.23), (NINTEDANIB:Blood glucose abnormal:0.33), (OXCARBAZEPINE:Blood glucose abnormal:1.35), (OXYBUTYNIN\OXYBUTYNIN CHLORIDE:Blood glucose abnormal:0.1), (PANCRELIPASE AMYLASE\PANCRELIPASE LIPASE\PANCRELIPASE PROTEASE:Blood glucose abnormal:0.03), (PANTOPRAZOLE MAGNESIUM:Blood glucose abnormal:1.58), (PATIROMER:Blood glucose abnormal:0.3), (PEGINTERFERON BETA-1A:Blood glucose abnormal:0.59), (PIROXICAM:Blood glucose abnormal:0.25), (POSACONAZOLE:Blood glucose abnormal:0.36), (PRAZOSIN HYDROCHLORIDE:Blood glucose abnormal:0.26), (PREDNISOLONE\PREDNISOLONE ACETATE\PREDNISOLONE SODIUM SUCCINATE:Blood glucose abnormal:0.29), (PROGESTERONE:Blood glucose abnormal:0.24), (PROPAFENONE HYDROCHLORIDE:Blood glucose abnormal:0.33), (PYRIDOXINE:Blood glucose abnormal:0.22), (RISEDRONATE SODIUM:Blood glucose abnormal:0.66), (ROFLUMILAST:Blood glucose abnormal:0.95), (SACCHAROMYCES CEREVISIAE:Blood glucose abnormal:0.06), (SALMETEROL XINAFOATE:Blood glucose abnormal:0.32), (SCOPOLAMINE:Blood glucose abnormal:0.57), (SENNOSIDES:Blood glucose abnormal:0.55), (SODIUM PHOSPHATE, DIBASIC\SODIUM PHOSPHATE, MONOBASIC:Blood glucose abnormal:0.33), (SUMATRIPTAN:Blood glucose abnormal:0.48), (SUMATRIPTAN\SUMATRIPTAN SUCCINATE:Blood glucose abnormal:1.15), (TERBINAFINE HYDROCHLORIDE:Blood glucose abnormal:0.37), (TETRACYCLINE:Blood glucose abnormal:0.91), (THALIDOMIDE:Blood glucose abnormal:0.35), (THYROID, PORCINE\THYROID, UNSPECIFIED:Blood glucose abnormal:0.09), (TOCILIZUMAB:Blood glucose abnormal:0.32), (UMECLIDINIUM BROMIDE:Blood glucose abnormal:0.03), (ZINC:Blood glucose abnormal:0.75), (AMIODARONE HYDROCHLORIDE:Blood glucose decreased:0.16), (AMITRIPTYLINE:Blood glucose decreased:0.29), (CALCITRIOL:Blood glucose decreased:0.41), (CALCIUM ACETATE:Blood glucose decreased:0.09), (CANAGLIFLOZIN\METFORMIN HYDROCHLORIDE:Blood glucose decreased:0.11), (CANDESARTAN:Blood glucose decreased:0.12), (CANDESARTAN CILEXETIL:Blood glucose decreased:0.14), (CERTOLIZUMAB PEGOL:Blood glucose decreased:0.31), (CHOLESTYRAMINE:Blood glucose decreased:0.32), (COLCHICINE:Blood glucose decreased:0.4), (CYCLOBENZAPRINE:Blood glucose decreased:0.34), (DICLOFENAC:Blood glucose decreased:0.12), (DOXYCYCLINE:Blood glucose decreased:0.14), (EMPAGLIFLOZIN\LINAGLIPTIN:Blood glucose decreased:0.49), (ENALAPRIL MALEATE:Blood glucose decreased:0.05), (ERYTHROPOIETIN:Blood glucose decreased:0.32), (ESZOPICLONE:Blood glucose decreased:0.06), (FERROUS SULFATE\FERROUS SULFATE, DRIED:Blood glucose decreased:0.88), (IBRUTINIB:Blood glucose decreased:0.86), (ILOPROST:Blood glucose decreased:0.22), (IRBESARTAN:Blood glucose decreased:0.27), (LERCANIDIPINE:Blood glucose decreased:0.04), (LOVASTATIN:Blood glucose decreased:0.58), (METHADONE HYDROCHLORIDE:Blood glucose decreased:0.04), (MOXIFLOXACIN HYDROCHLORIDE:Blood glucose decreased:0.31), (NEBIVOLOL:Blood glucose decreased:0.04), (NILOTINIB:Blood glucose decreased:0.02), (NITROFURANTOIN:Blood glucose decreased:0.08), (PANTOPRAZOLE MAGNESIUM:Blood glucose decreased:0.06), (PEGFILGRASTIM:Blood glucose decreased:0.28), (RANOLAZINE:Blood glucose decreased:0.83), (SACUBITRIL\VALSARTAN:Blood glucose decreased:0.21), (TERIFLUNOMIDE:Blood glucose decreased:0.17), (DEVICE:Blood glucose fluctuation:0.37), (EVOLOCUMAB:Blood glucose fluctuation:0.08), (.ALPHA.1-PROTEINASE INHIBITOR HUMAN:Blood glucose increased:0.36), (ACETAMINOPHEN\OXYCODONE HYDROCHLORIDE\OXYCODONE TEREPHTHALATE:Blood glucose increased:0.04), (ACETAMINOPHEN\OXYCODONE\OXYCODONE HYDROCHLORIDE:Blood glucose increased:0.58), (ACETAMINOPHEN\TRAMADOL:Blood glucose increased:0.05), (ACETAZOLAMIDE:Blood glucose increased:0.58), (ADO-TRASTUZUMAB EMTANSINE:Blood glucose increased:0.09), (AGALSIDASE BETA:Blood glucose increased:0.1), (ALCOHOL:Blood glucose increased:0.39), (AMIODARONE HYDROCHLORIDE:Blood glucose increased:1.31), (AMISULPRIDE:Blood glucose increased:0.27), (AMLODIPINE BESYLATE\ATORVASTATIN CALCIUM:Blood glucose increased:0.04), (AMLODIPINE BESYLATE\HYDROCHLOROTHIAZIDE\OLMESARTAN MEDOXOMIL:Blood glucose increased:0.06), (AZILSARTAN KAMEDOXOMIL:Blood glucose increased:0.04), (AZITHROMYCIN DIHYDRATE:Blood glucose increased:0.51), (BIMATOPROST:Blood glucose increased:0.17), (BUPRENORPHINE HYDROCHLORIDE:Blood glucose increased:0.24), (CALCIUM ACETATE:Blood glucose increased:1.42), (CANAKINUMAB:Blood glucose increased:0.29), (CARVEDILOL PHOSPHATE:Blood glucose increased:1.13), (CEFEPIME HYDROCHLORIDE:Blood glucose increased:0.12), (CEFOTAXIME SODIUM:Blood glucose increased:0.22), (CEFUROXIME:Blood glucose increased:0.1), (CETIRIZINE HYDROCHLORIDE\PSEUDOEPHEDRINE HYDROCHLORIDE:Blood glucose increased:0.28), (CLINDAMYCIN:Blood glucose increased:0.83), (DABIGATRAN:Blood glucose increased:0.24), (DACLIZUMAB:Blood glucose increased:1.25), (DANAZOL:Blood glucose increased:0.26), (DASATINIB:Blood glucose increased:1.09), (DESLANOSIDE:Blood glucose increased:0.22), (DEXTROAMPHETAMINE SULFATE:Blood glucose increased:0.02), (DIPYRONE:Blood glucose increased:0.12), (DOCUSATE:Blood glucose increased:0.81), (DOXEPIN:Blood glucose increased:0.14), (DOXEPIN HYDROCHLORIDE:Blood glucose increased:0.44), (DRONABINOL:Blood glucose increased:0.63), (EDOXABAN TOSYLATE:Blood glucose increased:0.59), (ELETRIPTAN HYDROBROMIDE:Blood glucose increased:1.15), (FESOTERODINE FUMARATE:Blood glucose increased:0.92), (FLUTICASONE FUROATE\SALMETEROL XINAFOATE:Blood glucose increased:0.83), (FORMOTEROL:Blood glucose increased:0.11), (GLYCOPYRRONIUM:Blood glucose increased:0.27), (GLYCOPYRRONIUM\INDACATEROL:Blood glucose increased:0.81), (HYDROXYCHLOROQUINE DIPHOSPHATE:Blood glucose increased:0.3), (HYDROXYUREA:Blood glucose increased:0.35), (ILOPROST:Blood glucose increased:0.18), (INFLUENZA VIRUS VACCINE:Blood glucose increased:0.85), (INSULIN PORK\INSULIN PURIFIED PORK:Blood glucose increased:0.02), (INVESTIGATIONAL PRODUCT:Blood glucose increased:0.17), (LEDIPASVIR\SOFOSBUVIR:Blood glucose increased:0.56), (LERCANIDIPINE:Blood glucose increased:1.67), (LEVOFLOXACIN HYDROCHLORIDE:Blood glucose increased:0.82), (LIDOCAINE\PRILOCAINE:Blood glucose increased:0.13), (LINEZOLID:Blood glucose increased:0.22), (LISDEXAMFETAMINE DIMESYLATE:Blood glucose increased:0.74), (LOMITAPIDE MESYLATE:Blood glucose increased:0.68), (MEGESTROL ACETATE:Blood glucose increased:0.17), (MEPOLIZUMAB:Blood glucose increased:0.42), (METHENAMINE HIPPURATE:Blood glucose increased:0.77), (METHYLPREDNISOLONE ACETATE:Blood glucose increased:1.12), (MUPIROCIN:Blood glucose increased:0.14), (MUPIROCIN\MUPIROCIN CALCIUM:Blood glucose increased:0.04), (NABUMETONE:Blood glucose increased:0.41), (NALOXONE HYDROCHLORIDE\OXYCODONE HYDROCHLORIDE:Blood glucose increased:0.9), (NITROFURANTOIN\NITROFURANTOIN MONOHYDRATE:Blood glucose increased:0.38), (NORTRIPTYLINE HYDROCHLORIDE:Blood glucose increased:0.15), (PAMIDRONATE DISODIUM:Blood glucose increased:0.06), (PATIROMER:Blood glucose increased:0.32), (PEGINTERFERON BETA-1A:Blood glucose increased:0.75), (PENTOSAN POLYSULFATE SODIUM:Blood glucose increased:0.12), (PHENYTOIN:Blood glucose increased:0.08), (PIROXICAM:Blood glucose increased:0.24), (PRAMIPEXOLE DIHYDROCHLORIDE:Blood glucose increased:0.83), (PRAZOSIN:Blood glucose increased:0.15), (PRAZOSIN HYDROCHLORIDE:Blood glucose increased:0.41), (PREDNISOLONE ACETATE:Blood glucose increased:0.11), (PROPAFENONE HYDROCHLORIDE:Blood glucose increased:0.14), (RETINOL:Blood glucose increased:0.05), (RIVASTIGMINE TARTRATE:Blood glucose increased:0.21), (RIZATRIPTAN:Blood glucose increased:0.06), (RIZATRIPTAN BENZOATE:Blood glucose increased:0.73), (SULBACTAM SODIUM:Blood glucose increased:0.51), (TETRACYCLINE:Blood glucose increased:0.57), (THALIDOMIDE:Blood glucose increased:0.52), (THEOPHYLLINE ANHYDROUS:Blood glucose increased:0.32), (TOBRAMYCIN:Blood glucose increased:0.63), (TOCOPHEROL:Blood glucose increased:0.16), (UMECLIDINIUM BROMIDE\VILANTEROL TRIFENATATE:Blood glucose increased:1.31), (VITAMIN A:Blood glucose increased:0.44), (ZINC:Blood glucose increased:0.74), (ACETAMINOPHEN\CODEINE PHOSPHATE:Blood iron:0.06), (ATROPINE SULFATE\DIPHENOXYLATE HYDROCHLORIDE:Blood iron:0.04), (BISOPROLOL FUMARATE:Blood iron:0.04), (CERTOLIZUMAB PEGOL:Blood iron:0.4), (CHOLESTYRAMINE:Blood iron:0.06), (DEVICE:Blood iron:0.19), (DEXLANSOPRAZOLE:Blood iron:0.75), (DILTIAZEM HYDROCHLORIDE:Blood iron:0.23), (EVOLOCUMAB:Blood iron:0.41), (IBRUTINIB:Blood iron:0.11), (ILOPROST:Blood iron:0.17), (LANSOPRAZOLE:Blood iron:0.29), (METOPROLOL TARTRATE:Blood iron:0.01), (PRAVASTATIN:Blood iron:0.31), (RAMIPRIL:Blood iron:0.25), (RIOCIGUAT:Blood iron:0.1), (VALSARTAN:Blood iron:0.33), (.ALPHA.1-PROTEINASE INHIBITOR HUMAN:Blood iron decreased:0.23), (AMIODARONE HYDROCHLORIDE:Blood iron decreased:0.53), (AMITRIPTYLINE HYDROCHLORIDE:Blood iron decreased:0.04), (AMLODIPINE BESYLATE\OLMESARTAN MEDOXOMIL:Blood iron decreased:0.04), (BENAZEPRIL HYDROCHLORIDE:Blood iron decreased:0.33), (BISACODYL:Blood iron decreased:0.2), (BISOPROLOL FUMARATE:Blood iron decreased:0.91), (BUDESONIDE\FORMOTEROL FUMARATE:Blood iron decreased:0.01), (CALCIUM ACETATE:Blood iron decreased:0.62), (CANDESARTAN:Blood iron decreased:0.74), (CARFILZOMIB:Blood iron decreased:0.13), (CEFTRIAXONE SODIUM:Blood iron decreased:0.09), (CLINDAMYCIN\CLINDAMYCIN PHOSPHATE:Blood iron decreased:0.62), (CLOTRIMAZOLE:Blood iron decreased:0.55), (CORTISONE\HYDROCORTISONE:Blood iron decreased:0.46), (DALFAMPRIDINE:Blood iron decreased:0.59), (DASATINIB:Blood iron decreased:0.85), (DESLORATADINE:Blood iron decreased:0.07), (DIETARY SUPPLEMENT:Blood iron decreased:0.17), (DIPHENHYDRAMINE:Blood iron decreased:0.78), (DOFETILIDE:Blood iron decreased:0.36), (DOXAZOSIN MESYLATE:Blood iron decreased:0.42), (EMPAGLIFLOZIN\LINAGLIPTIN:Blood iron decreased:0), (ENALAPRIL MALEATE:Blood iron decreased:0.73), (ENOXAPARIN:Blood iron decreased:0.69), (EPINEPHRINE:Blood iron decreased:0.45), (EVOLOCUMAB:Blood iron decreased:1.2), (FISH OIL\OMEGA-3 FATTY ACIDS:Blood iron decreased:0.03), (FLECAINIDE:Blood iron decreased:0.35), (FLUTICASONE:Blood iron decreased:0.29), (FLUTICASONE FUROATE:Blood iron decreased:0.02), (FLUTICASONE\FLUTICASONE PROPIONATE:Blood iron decreased:0.24), (FORMOTEROL FUMARATE DIHYDRATE\MOMETASONE FUROATE:Blood iron decreased:0.15), (HEPARIN SODIUM:Blood iron decreased:0.25), (ILOPROST:Blood iron decreased:0.1), (INDOMETHACIN:Blood iron decreased:0.38), (IPRATROPIUM:Blood iron decreased:0.48), (LEDIPASVIR\SOFOSBUVIR:Blood iron decreased:0.7), (LERCANIDIPINE:Blood iron decreased:0.78), (LIDOCAINE\PRILOCAINE:Blood iron decreased:0.15), (LINAGLIPTIN:Blood iron decreased:0.81), (LITHIUM CARBONATE:Blood iron decreased:0.11), (LOMITAPIDE MESYLATE:Blood iron decreased:0.19), (METHENAMINE HIPPURATE:Blood iron decreased:0.16), (METHYLPREDNISOLONE ACETATE:Blood iron decreased:0.02), (MIDODRINE:Blood iron decreased:0.05), (MOXIFLOXACIN HYDROCHLORIDE:Blood iron decreased:0.16), (NALOXONE HYDROCHLORIDE\OXYCODONE HYDROCHLORIDE:Blood iron decreased:0.24), (NEBIVOLOL:Blood iron decreased:0.03), (NILOTINIB:Blood iron decreased:0.8), (OLANZAPINE:Blood iron decreased:0.73), (PAROXETINE:Blood iron decreased:0.48), (PEGINTERFERON BETA-1A:Blood iron decreased:0.1), (PENICILLIN:Blood iron decreased:0.37), (PONATINIB:Blood iron decreased:0.21), (PRAMIPEXOLE:Blood iron decreased:0.36), (SACUBITRIL\VALSARTAN:Blood iron decreased:0.8), (SCOPOLAMINE:Blood iron decreased:0.21), (SECUKINUMAB:Blood iron decreased:0.75), (SULFAMETHOXAZOLE:Blood iron decreased:0.16), (TETRACYCLINE:Blood iron decreased:0.82), (THALIDOMIDE:Blood iron decreased:0.3), (THYROID, PORCINE:Blood iron decreased:0.37), (TOCILIZUMAB:Blood iron decreased:0.17), (TOLTERODINE TARTRATE:Blood iron decreased:0.05), (TRIAMCINOLONE ACETONIDE:Blood iron decreased:0.67), (.ALPHA.1-PROTEINASE INHIBITOR HUMAN:Blood magnesium decreased:0.08), (ACETAMINOPHEN\OXYCODONE\OXYCODONE HYDROCHLORIDE:Blood magnesium decreased:0.42), (ALEMTUZUMAB:Blood magnesium decreased:0.01), (AMLODIPINE\VALSARTAN:Blood magnesium decreased:0.07), (BICALUTAMIDE:Blood magnesium decreased:0.83), (BISACODYL OR DOCUSATE SODIUM:Blood magnesium decreased:1.12), (BUDESONIDE\FORMOTEROL FUMARATE:Blood magnesium decreased:0.16), (CANAGLIFLOZIN\METFORMIN HYDROCHLORIDE:Blood magnesium decreased:0.18), (CANAKINUMAB:Blood magnesium decreased:0.06), (CEFOTAXIME SODIUM:Blood magnesium decreased:0.2), (CHLORTHALIDONE:Blood magnesium decreased:0.19), (CHONDROITIN SULFATE (BOVINE)\GLUCOSAMINE HYDROCHLORIDE:Blood magnesium decreased:0.01), (CIMETIDINE:Blood magnesium decreased:0.37), (CLOZAPINE:Blood magnesium decreased:0.22), (DACLIZUMAB:Blood magnesium decreased:0.46), (DASATINIB:Blood magnesium decreased:1.15), (DOCUSATE CALCIUM:Blood magnesium decreased:0.25), (DOXAZOSIN MESYLATE:Blood magnesium decreased:0.46), (DULAGLUTIDE:Blood magnesium decreased:0.31), (ELETRIPTAN HYDROBROMIDE:Blood magnesium decreased:0.51), (ERYTHROMYCIN:Blood magnesium decreased:0.29), (EVOLOCUMAB:Blood magnesium decreased:0.93), (EXEMESTANE:Blood magnesium decreased:0.22), (FINGOLIMOD HYDROCHLORIDE:Blood magnesium decreased:0.08), (FORMOTEROL FUMARATE:Blood magnesium decreased:0.25), (FORMOTEROL FUMARATE\MOMETASONE FUROATE:Blood magnesium decreased:0), (GLYCOPYRRONIUM\ANDACATEROL:Blood magnesium decreased:0.38), (HERBALS\TURMERIC:Blood magnesium decreased:0.01), (HYOSCYAMINE:Blood magnesium decreased:0.29), (ILOPROST:Blood magnesium decreased:0.19), (IMIPENEM:Blood magnesium decreased:0.3), (IMIPRAMINE HYDROCHLORIDE:Blood magnesium decreased:0.48), (INDAPAMIDE\PERINDOPRIL:Blood magnesium decreased:0.46), (INDOMETHACIN:Blood magnesium decreased:0.2), (INSULIN GLULISINE:Blood magnesium decreased:0.19), (ISONIAZID:Blood magnesium decreased:0.45), (KETOROLAC\KETOROLAC TROMETHAMINE:Blood magnesium decreased:0), (LABETALOL\LABETALOL HYDROCHLORIDE:Blood magnesium decreased:1.02), (LEDIPASVIR\SOFOSBUVIR:Blood magnesium decreased:0.28), (LERCANIDIPINE HYDROCHLORIDE:Blood magnesium decreased:0.28), (LINACLOTIDE:Blood magnesium decreased:0.13), (LITHIUM CARBONATE:Blood magnesium decreased:0.27), (LOMITAPIDE MESYLATE:Blood magnesium decreased:0.1), (METAXALONE:Blood magnesium decreased:1.11), (METHENAMINE HIPPURATE:Blood magnesium decreased:0.64), (MUPIROCIN\MUPIROCIN CALCIUM:Blood magnesium decreased:0.16), (NADOLOL:Blood magnesium decreased:0.26), (NATEGLINIDE:Blood magnesium decreased:0.14), (NEBIVOLOL:Blood magnesium decreased:0.34), (NILOTINIB:Blood magnesium decreased:1.19), (PANTOPRAZOLE MAGNESIUM:Blood magnesium decreased:1.06), (PATIROMER:Blood magnesium decreased:0.03), (PAZOPANIB HYDROCHLORIDE:Blood magnesium decreased:0.06), (PENICILLIN:Blood magnesium decreased:0.63), (PHENOBARBITAL:Blood magnesium decreased:0.13), (PRIMIDONE:Blood magnesium decreased:0.78), (PROGESTERONE:Blood magnesium decreased:0.11), (PYRIDOXINE:Blood magnesium decreased:0.28), (QUETIAPINE:Blood magnesium decreased:0.45), (RALOXIFENE HYDROCHLORIDE:Blood magnesium decreased:0.31), (RIZATRIPTAN BENZOATE:Blood magnesium decreased:0.24), (SECUKINUMAB:Blood magnesium decreased:0.18), (SODIUM PHOSPHATE, DIBASIC\SODIUM PHOSPHATE, MONOBASIC:Blood magnesium decreased:0.11), (SULFAMETHOXAZOLE:Blood magnesium decreased:0.62), (SUMATRIPTAN\SUMATRIPTAN SUCCINATE:Blood magnesium decreased:0.48), (TERAZOSIN HYDROCHLORIDE:Blood magnesium decreased:0.05), (TERBUTALINE SULFATE:Blood magnesium decreased:0.14), (TIGECYCLINE:Blood magnesium decreased:0.13), (TIMOLOL:Blood magnesium decreased:0.52), (TINZAPARIN SODIUM:Blood magnesium decreased:0.01), (TIOTROPIUM:Blood magnesium decreased:0.38), (TOLTERODINE TARTRATE:Blood magnesium decreased:0.23), (VANCOMYCIN HYDROCHLORIDE:Blood magnesium decreased:0.73), (VARENICLINE TARTRATE:Blood magnesium decreased:1.08), (VITAMIN A:Blood magnesium decreased:0.02), (ENALAPRIL:Blood phosphorus increased:0.27), (IBRUTINIB:Blood phosphorus increased:0.03), (RAMIPRIL:Blood phosphorus increased:0.23), (AMPHETAMINE ASPARTATE\AMPHETAMINE SULFATE\DEXTROAMPHETAMINE SACCHARATE\DEXTROAMPHETAMINE SULFATE:Blood potassium:0.12), (BISACODYL OR DOCUSATE SODIUM:Blood potassium:0.13), (CALCIUM CARBONATE\CHOLECALCIFEROL:Blood potassium:0.55), (CANDESARTAN:Blood potassium:0.37), (CANDESARTAN CILEXETIL:Blood potassium:0.25), (CARFILZOMIB:Blood potassium:0.23), (CHOLESTYRAMINE:Blood potassium:0.03), (CLINDAMYCIN\CLINDAMYCIN PHOSPHATE:Blood potassium:0.53), (CLOTRIMAZOLE:Blood potassium:0.06), (CYCLOBENZAPRINE:Blood potassium:0.21), (DARBEPOETIN ALFA:Blood potassium:0.36), (DASATINIB:Blood potassium:0.12), (DICLOFENAC:Blood potassium:0.19), (DICLOFENAC SODIUM:Blood potassium:0.51), (DIVALPROEX SODIUM:Blood potassium:0.2), (EPINEPHRINE:Blood potassium:0.4), (ESOMEPRAZOLE:Blood potassium:0.22), (EVOLOCUMAB:Blood potassium:0.84), (FLECAINIDE:Blood potassium:0.3), (FLUTICASONE:Blood potassium:0.07), (LATANOPROST:Blood potassium:0.05), (LINAGLIPTIN:Blood potassium:0.06), (LORATADINE:Blood potassium:0.52), (METAXALONE:Blood potassium:0.17), (NAPROXEN SODIUM:Blood potassium:0.04), (NILOTINIB:Blood potassium:0.19), (NITROFURANTOIN:Blood potassium:0.32), (PANTOPRAZOLE MAGNESIUM:Blood potassium:0.23), (PAROXETINE\PAROXETINE HYDROCHLORIDE:Blood potassium:0.09), (PRIMIDONE:Blood potassium:0.16), (RANOLAZINE:Blood potassium:1.13), (SECUKINUMAB:Blood potassium:0.21), (TERIFLUNOMIDE:Blood potassium:1.06), (THALIDOMIDE:Blood potassium:0), (TRIAMCINOLONE ACETONIDE:Blood potassium:0.12), (ACARBOSE:Blood potassium abnormal:0.08), (ACETAMINOPHEN\BUTALBITAL\CAFFEINE:Blood potassium abnormal:0.45), (AMPHETAMINE ASPARTATE\AMPHETAMINE SULFATE\DEXTROAMPHETAMINE SACCHARATE\DEXTROAMPHETAMINE SULFATE:Blood potassium abnormal:0.53), (BISACODYL:Blood potassium abnormal:0), (CALCIUM ACETATE:Blood potassium abnormal:0.35), (CANDESARTAN CILEXETIL:Blood potassium abnormal:0.37), (CANDESARTAN CILEXETIL\HYDROCHLOROTHIAZIDE:Blood potassium abnormal:0.12), (CARFILZOMIB:Blood potassium abnormal:0.11), (CERTOLIZUMAB PEGOL:Blood potassium abnormal:0.14), (CINACALCET HYDROCHLORIDE:Blood potassium abnormal:0.23), (CLINDAMYCIN\CLINDAMYCIN PHOSPHATE:Blood potassium abnormal:0.87), (COLESEVELAM HYDROCHLORIDE:Blood potassium abnormal:0.09), (DAPAGLIFLOZIN PROPANEDIOL:Blood potassium abnormal:0.22), (DASATINIB:Blood potassium abnormal:0.27), (DEVICE:Blood potassium abnormal:0.58), (DIVALPROEX SODIUM:Blood potassium abnormal:0.69), (DOXYCYCLINE:Blood potassium abnormal:0.21), (DUTASTERIDE:Blood potassium abnormal:0.15), (ELETRIPTAN HYDROBROMIDE:Blood potassium abnormal:0.24), (ERYTHROPOIETIN:Blood potassium abnormal:0.37), (EVEROLIMUS:Blood potassium abnormal:0.39), (EVOLOCUMAB:Blood potassium abnormal:0.73), (EZETIMIBE\SIMVASTATIN:Blood potassium abnormal:0.14), (HYDROCHLOROTHIAZIDE\LISINOPRIL:Blood potassium abnormal:0.36), (HYDROXYZINE PAMOATE:Blood potassium abnormal:0.52), (IBRUTINIB:Blood potassium abnormal:1.13), (ILOPROST:Blood potassium abnormal:0.36), (LACOSAMIDE:Blood potassium abnormal:0.14), (LIDOCAINE HYDROCHLORIDE:Blood potassium abnormal:0.26), (LIDOCAINE\PRILOCAINE:Blood potassium abnormal:0.1), (LISDEXAMFETAMINE DIMESYLATE:Blood potassium abnormal:0.02), (METAXALONE:Blood potassium abnormal:0.38), (METRONIDAZOLE:Blood potassium abnormal:0.21), (MODAFINIL:Blood potassium abnormal:0.18), (PANTOPRAZOLE MAGNESIUM:Blood potassium abnormal:0.1), (PAROXETINE\PAROXETINE HYDROCHLORIDE:Blood potassium abnormal:0.1), (POLYETHYLENE GLYCOLS:Blood potassium abnormal:0.66), (PRIMIDONE:Blood potassium abnormal:0.79), (SULFAMETHOXAZOLE:Blood potassium abnormal:0.07), (TELMISARTAN:Blood potassium abnormal:0.22), (TERIFLUNOMIDE:Blood potassium abnormal:0.38), (TIMOLOL:Blood potassium abnormal:0.17), (TIZANIDINE HYDROCHLORIDE:Blood potassium abnormal:0.6), (UMECLIDINIUM BROMIDE\VILANTEROL TRIFENATATE:Blood potassium abnormal:0.4), (VALACYCLOVIR HYDROCHLORIDE:Blood potassium abnormal:0.09), (VARENICLINE TARTRATE:Blood potassium abnormal:0.08), (VITAMIN B:Blood potassium abnormal:0.57), (ZOLPIDEM\ZOLPIDEM TARTRATE:Blood potassium abnormal:0.87), (ZONISAMIDE:Blood potassium abnormal:0.01), (.ALPHA.1-PROTEINASE INHIBITOR HUMAN:Blood potassium decreased:0.33), (ACETAMINOPHEN\PROPOXYPHENE HYDROCHLORIDE:Blood potassium decreased:0.26), (ACETAMINOPHEN\PROPOXYPHENE NAPSYLATE:Blood potassium decreased:0.15), (AGALSIDASE BETA:Blood potassium decreased:0.26), (ALCOHOL:Blood potassium decreased:0.48), (ALEMTUZUMAB:Blood potassium decreased:0.39), (ALFUZOSIN HYDROCHLORIDE:Blood potassium decreased:0.17), (ALISKIREN HEMIFUMARATE:Blood potassium decreased:0.23), (AMANTADINE HYDROCHLORIDE:Blood potassium decreased:0.04), (AMINO ACIDS\DEXTROSE\ELECTROLYTES NOS:Blood potassium decreased:0.17), (AMISULPRIDE:Blood potassium decreased:0.59), (AMLODIPINE\VALSARTAN:Blood potassium decreased:0.49), (ANTIHISTAMINES NOS:Blood potassium decreased:0.12), (ASPIRIN LYSINE:Blood potassium decreased:0.01), (BETAMETHASONE DIPROPIONATE:Blood potassium decreased:0.07), (BETAMETHASONE DIPROPIONATE\CALCIPOTRIENE\CALCIPOTRIENE HYDRATE:Blood potassium decreased:0.04), (BETAMETHASONE\CALCIPOTRIENE:Blood potassium decreased:0), (BROMAZEPAM:Blood potassium decreased:0.29), (BUPRENORPHINE HYDROCHLORIDE:Blood potassium decreased:0.52), (BUTYLSCOPOLAMINE BROMIDE:Blood potassium decreased:0.38), (CALCIUM CARBONATE\VITAMIN D:Blood potassium decreased:0.59), (CALCIUM\MAGNESIUM:Blood potassium decreased:0.27), (CALCIUM\MAGNESIUM\ZINC:Blood potassium decreased:0.3), (CANAGLIFLOZIN\METFORMIN HYDROCHLORIDE:Blood potassium decreased:0.69), (CANAKINUMAB:Blood potassium decreased:0.53), (CEFOTAXIME SODIUM:Blood potassium decreased:0.58), (CHLORAMBUCIL:Blood potassium decreased:0.29), (CHLORDIAZEPOXIDE HYDROCHLORIDE\CLIDINIUM BROMIDE:Blood potassium decreased:0.22), (CIMETIDINE:Blood potassium decreased:1.37), (CLINDAMYCIN:Blood potassium decreased:1.55), (CLOBAZAM:Blood potassium decreased:0.17), (COD LIVER OIL:Blood potassium decreased:0.46), (COLISTIMETHATE SODIUM:Blood potassium decreased:0), (CYPROHEPTADINE:Blood potassium decreased:0.66), (DABIGATRAN:Blood potassium decreased:0.26), (DACLIZUMAB:Blood potassium decreased:1.05), (DEFLAZACORT:Blood potassium decreased:0.23), (DEXAMETHASONE ACETATE:Blood potassium decreased:0.16), (DIGITOXIN:Blood potassium decreased:0.02), (DIPYRONE:Blood potassium decreased:0.43), (DOLASETRON MESYLATE:Blood potassium decreased:0.21), (DOXYCYCLINE HYCLATE:Blood potassium decreased:0.36), (DULAGLUTIDE:Blood potassium decreased:0.1), (EDOXABAN TOSYLATE:Blood potassium decreased:0.74), (ELTROMBOPAG OLAMINE:Blood potassium decreased:0.05), (EMPAGLIFLOZIN\LINAGLIPTIN:Blood potassium decreased:0.84), (ESTROGENS, CONJUGATED\MEDROXYPROGESTERONE ACETATE:Blood potassium decreased:0.77), (EVOLOCUMAB:Blood potassium decreased:1.66), (FINGOLIMOD HYDROCHLORIDE:Blood potassium decreased:0.66), (FLUTICASONE FUROATE:Blood potassium decreased:0.75), (FLUVOXAMINE MALEATE:Blood potassium decreased:0.15), (FORMOTEROL:Blood potassium decreased:0.62), (FORMOTEROL FUMARATE:Blood potassium decreased:0.84), (FORMOTEROL FUMARATE\MOMETASONE FUROATE:Blood potassium decreased:0.41), (GIMERACIL\OTERACIL\TEGAFUR:Blood potassium decreased:0.37), (GLYCOPYRRONIUM:Blood potassium decreased:0.05), (HYDROXYCHLOROQUINE DIPHOSPHATE:Blood potassium decreased:0.28), (HYOSCYAMINE:Blood potassium decreased:1.18), (ILOPROST:Blood potassium decreased:0.96), (IMIPENEM:Blood potassium decreased:0.39), (INDACATEROL:Blood potassium decreased:0.11), (INDAPAMIDE\PERINDOPRIL:Blood potassium decreased:0.56), (INSULIN DEGLUDEC:Blood potassium decreased:0.09), (INSULIN GLULISINE:Blood potassium decreased:0.17), (INTERFERON ALFA-2A:Blood potassium decreased:0.15), (INTERFERON ALFA-2B:Blood potassium decreased:0.32), (INTERFERON GAMMA-1B:Blood potassium decreased:0.3), (IRINOTECAN HYDROCHLORIDE:Blood potassium decreased:0.54), (IXAZOMIB:Blood potassium decreased:0.47), (LACTOBACILLUS RHAMNOSUS:Blood potassium decreased:0.52), (LEDIPASVIR\SOFOSBUVIR:Blood potassium decreased:1.56), (LERCANIDIPINE:Blood potassium decreased:1.08), (LEVALBUTEROL:Blood potassium decreased:0.03), (LEVOLEUCOVORIN CALCIUM:Blood potassium decreased:0.06), (LITHIUM CARBONATE:Blood potassium decreased:1.21), (LOMITAPIDE MESYLATE:Blood potassium decreased:0.76), (MEFENAMIC ACID:Blood potassium decreased:0.04), (MEPOLIZUMAB:Blood potassium decreased:0.06), (MESNA:Blood potassium decreased:0.42), (METFORMIN HYDROCHLORIDE\VILDAGLIPTIN:Blood potassium decreased:0.2), (NATEGLINIDE:Blood potassium decreased:0.55), (NOREPINEPHRINE BITARTRATE:Blood potassium decreased:0.05), (OCTREOTIDE:Blood potassium decreased:0.18), (OLOPATADINE:Blood potassium decreased:0.11), (OMEPRAZOLE SODIUM:Blood potassium decreased:0.24), (OXYMETAZOLINE:Blood potassium decreased:0.08), (PANTOPRAZOLE MAGNESIUM:Blood potassium decreased:1.55), (PARICALCITOL:Blood potassium decreased:0.17), (PATIROMER:Blood potassium decreased:0.46), (PEMETREXED:Blood potassium decreased:0.46), (PEMETREXED DISODIUM:Blood potassium decreased:0.67), (PHENYTOIN SODIUM:Blood potassium decreased:0.34), (PIMECROLIMUS:Blood potassium decreased:0.06), (PIOGLITAZONE:Blood potassium decreased:0.11), (PIRFENIDONE:Blood potassium decreased:0.07), (PIROXICAM:Blood potassium decreased:0.5), (PRAZOSIN HYDROCHLORIDE:Blood potassium decreased:0.36), (PREDNISOLONE SODIUM PHOSPHATE:Blood potassium decreased:0.22), (RADIATION THERAPY:Blood potassium decreased:0.45), (RALOXIFENE HYDROCHLORIDE:Blood potassium decreased:1.21), (REGORAFENIB:Blood potassium decreased:0.35), (RIVASTIGMINE:Blood potassium decreased:0.15), (SACCHAROMYCES CEREVISIAE:Blood potassium decreased:0.41), (SALMETEROL XINAFOATE:Blood potassium decreased:0.31), (SECUKINUMAB:Blood potassium decreased:1.11), (SITAGLIPTIN:Blood potassium decreased:0.24), (SULFAMETHOXAZOLE:Blood potassium decreased:1.16), (TEDUGLUTIDE\WATER:Blood potassium decreased:0.06), (TELMISARTAN:Blood potassium decreased:1.86), (TERBINAFINE HYDROCHLORIDE:Blood potassium decreased:0.56), (TERIFLUNOMIDE:Blood potassium decreased:0.7), (TETRACYCLINE:Blood potassium decreased:1.44), (TIOTROPIUM:Blood potassium decreased:0.87), (VALGANCICLOVIR HYDROCHLORIDE:Blood potassium decreased:1.15), (VANCOMYCIN HYDROCHLORIDE:Blood potassium decreased:0.85), (VEMURAFENIB:Blood potassium decreased:0.05), (VINORELBINE TARTRATE:Blood potassium decreased:0.21), (ZAFIRLUKAST:Blood potassium decreased:0.37), (ZIPRASIDONE HYDROCHLORIDE\ZIPRASIDONE MESYLATE:Blood potassium decreased:0.09), (ACARBOSE:Blood potassium increased:0.02), (BISACODYL:Blood potassium increased:0.05), (CANDESARTAN:Blood potassium increased:0.57), (CANDESARTAN CILEXETIL:Blood potassium increased:0.72), (CEPHALEXIN:Blood potassium increased:0.74), (CHOLESTYRAMINE:Blood potassium increased:0.06), (CLINDAMYCIN\CLINDAMYCIN PHOSPHATE:Blood potassium increased:0.52), (COLCHICINE:Blood potassium increased:0.19), (DASATINIB:Blood potassium increased:0.09), (DEVICE:Blood potassium increased:0.24), (DICYCLOMINE HYDROCHLORIDE:Blood potassium increased:0.21), (DULAGLUTIDE:Blood potassium increased:0.25), (ESZOPICLONE:Blood potassium increased:0.04), (EVEROLIMUS:Blood potassium increased:0.31), (EVOLOCUMAB:Blood potassium increased:0.53), (FLECAINIDE:Blood potassium increased:0.09), (FLUTICASONE:Blood potassium increased:0.01), (HYDROCHLOROTHIAZIDE\LISINOPRIL:Blood potassium increased:0.32), (HYDROCODONE:Blood potassium increased:0.3), (ILOPROST:Blood potassium increased:0.03), (LACOSAMIDE:Blood potassium increased:0.15), (LEDIPASVIR\SOFOSBUVIR:Blood potassium increased:0.1), (LEVALBUTEROL HYDROCHLORIDE:Blood potassium increased:0.25), (MELOXICAM:Blood potassium increased:0.49), (METRONIDAZOLE:Blood potassium increased:0.39), (MYCOPHENOLATE MOFETIL\MYCOPHENOLATE MOFETIL HYDROCHLORIDE:Blood potassium increased:0.04), (NAPROXEN:Blood potassium increased:0.57), (NAPROXEN SODIUM:Blood potassium increased:0.02), (NEBIVOLOL:Blood potassium increased:0.06), (OXCARBAZEPINE:Blood potassium increased:0.66), (PRAMIPEXOLE DIHYDROCHLORIDE:Blood potassium increased:0.06), (RAMIPRIL:Blood potassium increased:1.06), (RANOLAZINE:Blood potassium increased:0.94), (SODIUM OXYBATE:Blood potassium increased:0.08), (SOTALOL:Blood potassium increased:0.24), (TERIFLUNOMIDE:Blood potassium increased:0.34), (TOPIRAMATE:Blood potassium increased:0.89), (UMECLIDINIUM BROMIDE\VILANTEROL TRIFENATATE:Blood potassium increased:0.11), (.ALPHA.1-PROTEINASE INHIBITOR HUMAN:Blood pressure:0.1), (ABOBOTULINUMTOXINA:Blood pressure:0.11), (ACENOCOUMAROL:Blood pressure:0.69), (ACETAMINOPHEN\CAFFEINE:Blood pressure:0), (ACETAMINOPHEN\DICHLORALPHENAZONE\ISOMETHEPTENE:Blood pressure:0.03), (ACETAMINOPHEN\PROPOXYPHENE NAPSYLATE:Blood pressure:0.96), (ACETAMINOPHEN\TRAMADOL HYDROCHLORIDE:Blood pressure:0.27), (ADO-TRASTUZUMAB EMTANSINE:Blood pressure:0.64), (ALBIGLUTIDE:Blood pressure:0.2), (ALENDRONIC ACID:Blood pressure:1.2), (ALFACALCIDOL:Blood pressure:0.86), (ALUMINUM HYDROXIDE\DIMETHICONE\MAGNESIUM HYDROXIDE:Blood pressure:0.83), (AMANTADINE HYDROCHLORIDE:Blood pressure:0.7), (AMINO ACIDS\DEXTROSE\ELECTROLYTES NOS:Blood pressure:0.24), (AMMONIUM CHLORIDE\DEXTROMETHORPHAN\SODIUM CITRATE:Blood pressure:0.09), (ANTIHISTAMINES NOS:Blood pressure:0.44), (APREPITANT:Blood pressure:0.13), (ARIPIPRAZOLE LAUROXIL:Blood pressure:0.4), (ATOVAQUONE:Blood pressure:0.03), (ATROPINE\DIPHENOXYLATE:Blood pressure:0.32), (BELATACEPT:Blood pressure:0.04), (BETAMETHASONE DIPROPIONATE:Blood pressure:0.32), (BETAMETHASONE DIPROPIONATE\CLOTRIMAZOLE:Blood pressure:0.48), (BETHANECHOL CHLORIDE:Blood pressure:0.13), (BUPRENORPHINE HYDROCHLORIDE:Blood pressure:1.15), (CALCIUM\MAGNESIUM\ZINC:Blood pressure:1.24), (CANAKINUMAB:Blood pressure:0.54), (CARBOMER:Blood pressure:0.11), (CASANTHRANOL\DOCUSATE SODIUM:Blood pressure:0.38), (CEFAZOLIN:Blood pressure:0.46), (CEFAZOLIN SODIUM:Blood pressure:0.04), (CERIVASTATIN SODIUM:Blood pressure:0.16), (CHLORHEXIDINE GLUCONATE:Blood pressure:0.64), (CHLORPHENIRAMINE\HYDROCODONE:Blood pressure:0.3), (CHONDROITIN SULFATE (BOVINE)\GLUCOSAMINE HYDROCHLORIDE:Blood pressure:1.06), (CILASTATIN SODIUM\IMIPENEM:Blood pressure:0.2), (CLINDAMYCIN:Blood pressure:1.62), (CURCUMIN:Blood pressure:0.1), (DABIGATRAN:Blood pressure:0.88), (DACLIZUMAB:Blood pressure:1.4), (DAPAGLIFLOZIN PROPANEDIOL\METFORMIN HYDROCHLORIDE:Blood pressure:0.1), (DEFLAZACORT:Blood pressure:1.43), (DIMENHYDRINATE:Blood pressure:1.14), (DOLASETRON MESYLATE:Blood pressure:0.22), (DOXEPIN HYDROCHLORIDE:Blood pressure:1.07), (ECONAZOLE:Blood pressure:0.38), (EDOXABAN TOSYLATE:Blood pressure:1.36), (EFINACONAZOLE:Blood pressure:0.13), (EMPAGLIFLOZIN\LINAGLIPTIN:Blood pressure:1.08), (EPIRUBICIN:Blood pressure:0.01), (ESTRIOL:Blood pressure:0.22), (ESTROGENS:Blood pressure:0.8), (ETHINYL ESTRADIOL\NORGESTREL:Blood pressure:0.63), (EVOLOCUMAB:Blood pressure:2.1), (FENOTEROL HYDROBROMIDE:Blood pressure:0.23), (FERROUS BISGLYCINATE:Blood pressure:0.32), (FERROUS SULFATE:Blood pressure:1.93), (FLUINDIONE:Blood pressure:0.07), (FLUTICASONE:Blood pressure:1.42), (FLUVOXAMINE MALEATE:Blood pressure:0.59), (FORMOTEROL:Blood pressure:0.41), (FORMOTEROL FUMARATE\MOMETASONE FUROATE:Blood pressure:0.58), (FROVATRIPTAN SUCCINATE:Blood pressure:0.1), (FUSIDIC ACID:Blood pressure:0.54), (GLIMEPIRIDE\ROSIGLITAZONE MALEATE:Blood pressure:0.14), (GLYCOPYRROLATE\INDACATEROL MALEATE:Blood pressure:0.15), (GUANFACINE HYDROCHLORIDE:Blood pressure:0.55), (HERBALS\TURMERIC:Blood pressure:1.15), (HYDROCORTISONE SODIUM SUCCINATE:Blood pressure:0.2), (HYDROXYCHLOROQUINE DIPHOSPHATE:Blood pressure:0.54), (HYDROXYUREA:Blood pressure:0.61), (ICOSAPENT ETHYL:Blood pressure:0.11), (ILOPROST:Blood pressure:0.49), (INFLIXIMAB-DYYB:Blood pressure:0.02), (INSULIN DEGLUDEC:Blood pressure:0.84), (INSULIN PORK\INSULIN PURIFIED PORK:Blood pressure:0.08), (INTERFERON:Blood pressure:0.31), (INTERFERON ALFA-2A:Blood pressure:0.33), (INTERFERON GAMMA-1B:Blood pressure:0.05), (IRON SUCROSE:Blood pressure:0.36), (ISOPROPYL ALCOHOL\LEUPROLIDE ACETATE\NORETHINDRONE ACETATE:Blood pressure:0.15), (LANREOTIDE ACETATE:Blood pressure:0.18), (LAPATINIB DITOSYLATE:Blood pressure:0.22), (LURASIDONE HYDROCHLORIDE:Blood pressure:0.5), (MAGNESIUM GLUCONATE:Blood pressure:0.34), (MEFENAMIC ACID:Blood pressure:0.49), (MEPERIDINE:Blood pressure:0.36), (MEPOLIZUMAB:Blood pressure:0.28), (MINOCYCLINE HYDROCHLORIDE:Blood pressure:0.44), (MORPHINE SULFATE\NALTREXONE HYDROCHLORIDE:Blood pressure:0.12), (NALOXEGOL OXALATE:Blood pressure:0.04), (NALOXONE HYDROCHLORIDE\OXYCODONE HYDROCHLORIDE:Blood pressure:1.35), (NALOXONE HYDROCHLORIDE\TILIDINE HYDROCHLORIDE:Blood pressure:0.58), (NINTEDANIB:Blood pressure:0.57), (NIRAPARIB:Blood pressure:0.3), (OCTREOTIDE:Blood pressure:0.14), (OLODATEROL HYDROCHLORIDE\TIOTROPIUM BROMIDE MONOHYDRATE:Blood pressure:1.13), (OMEPRAZOLE SODIUM:Blood pressure:0.29), (OSELTAMIVIR:Blood pressure:0.08), (PALBOCICLIB:Blood pressure:0.3), (PANTOPRAZOLE:Blood pressure:2.82), (PARICALCITOL:Blood pressure:1), (PAROXETINE:Blood pressure:1.24), (PATIROMER:Blood pressure:0.66), (PEGINTERFERON BETA-1A:Blood pressure:1.17), (PENICILLIN G:Blood pressure:0.24), (PENICILLIN V:Blood pressure:0.19), (PENTOXIFYLLINE:Blood pressure:0.8), (PHYTONADIONE:Blood pressure:0.4), (PIMECROLIMUS:Blood pressure:0.68), (PONATINIB:Blood pressure:1.35), (POVIDONE:Blood pressure:0.12), (PRASTERONE:Blood pressure:0.46), (PRAVASTATIN:Blood pressure:3.09), (PREDNISOLONE SODIUM PHOSPHATE:Blood pressure:0.12), (PROTRIPTYLINE HYDROCHLORIDE:Blood pressure:0.64), (PYRIDOSTIGMINE BROMIDE:Blood pressure:0.7), (QUINAPRIL:Blood pressure:0.7), (RANITIDINE:Blood pressure:1.52), (REGORAFENIB:Blood pressure:0.22), (RIZATRIPTAN:Blood pressure:0.6), (ROFECOXIB:Blood pressure:0.03), (SACUBITRIL\VALSARTAN:Blood pressure:0.83), (SALMETEROL:Blood pressure:0.27), (SALMETEROL XINAFOATE:Blood pressure:0.65), (SCOPOLAMINE:Blood pressure:1.34), (SEVOFLURANE:Blood pressure:0.47), (SODIUM PHOSPHATE, DIBASIC\SODIUM PHOSPHATE, MONOBASIC:Blood pressure:0.48), (SOTALOL HYDROCHLORIDE:Blood pressure:0.3), (TAFLUPROST:Blood pressure:0.19), (TAMOXIFEN CITRATE:Blood pressure:0.5), (TASIMELTEON:Blood pressure:0.02), (TEDUGLUTIDE\WATER:Blood pressure:0.13), (TERBUTALINE SULFATE:Blood pressure:1.56), (THYROID, PORCINE:Blood pressure:1.62), (TIMOLOL MALEATE\TRAVOPROST:Blood pressure:0.25), (TIMOLOL\TRAVOPROST:Blood pressure:0.02), (TOCOPHEROL:Blood pressure:0.82), (TRIMIPRAMINE MALEATE:Blood pressure:0.04), (UBIQUINOL:Blood pressure:0), (UMECLIDINIUM BROMIDE:Blood pressure:0.48), (UMECLIDINIUM BROMIDE\VILANTEROL TRIFENATATE:Blood pressure:1.25), (VINORELBINE TARTRATE:Blood pressure:0.07), (YEAST:Blood pressure:0.37), (ZILEUTON:Blood pressure:0.37), (ZINC:Blood pressure:1.32), (ZOLMITRIPTAN:Blood pressure:0.57), (.ALPHA.1-PROTEINASE INHIBITOR HUMAN:Blood pressure abnormal:0.63), (ABOBOTULINUMTOXINA:Blood pressure abnormal:0.29), (ACETAMINOPHEN\CAFFEINE:Blood pressure abnormal:0.75), (ACETAMINOPHEN\TRAMADOL:Blood pressure abnormal:1.25), (ADO-TRASTUZUMAB EMTANSINE:Blood pressure abnormal:1.38), (AGALSIDASE BETA:Blood pressure abnormal:2.01), (ALFALFA:Blood pressure abnormal:0.05), (ALISKIREN HEMIFUMARATE\VALSARTAN:Blood pressure abnormal:0.2), (ALMOTRIPTAN MALATE:Blood pressure abnormal:0.13), (AMILORIDE HYDROCHLORIDE\FUROSEMIDE:Blood pressure abnormal:0.07), (AMINO ACIDS\DEXTROSE\ELECTROLYTES NOS:Blood pressure abnormal:1.01), (AMMONIUM CHLORIDE\DEXTROMETHORPHAN\SODIUM CITRATE:Blood pressure abnormal:0.77), (AMOXICILLIN\CLARITHROMYCIN\ESOMEPRAZOLE:Blood pressure abnormal:0.52), (APREPITANT:Blood pressure abnormal:0.76), (ASCORBIC ACID/CHROMIUM/SELENIUM/VANADYL SULFATE:Blood pressure abnormal:0.4), (ASIAN GINSENG:Blood pressure abnormal:0.5), (ASPIRIN\CAFFEINE\SALICYLAMIDE:Blood pressure abnormal:0.34), (AZITHROMYCIN MONOHYDRATE:Blood pressure abnormal:1.07), (BALSALAZIDE DISODIUM:Blood pressure abnormal:0.77), (BECLOMETHASONE:Blood pressure abnormal:0.69), (BELATACEPT:Blood pressure abnormal:0.25), (BENSERAZIDE HYDROCHLORIDE\LEVODOPA:Blood pressure abnormal:0.25), (BETAMETHASONE DIPROPIONATE\CALCIPOTRIENE\CALCIPOTRIENE HYDRATE:Blood pressure abnormal:1.53), (BETAMETHASONE\CALCIPOTRIENE:Blood pressure abnormal:0.83), (BETAXOLOL HYDROCHLORIDE:Blood pressure abnormal:0.08), (BISACODYL\GLYCERIN\MINERAL OIL\SODIUM PHOSPHATE, DIBASIC\SODIUM PHOSPHATE, MONOBASIC:Blood pressure abnormal:0.15), (BOCEPREVIR:Blood pressure abnormal:0.26), (BOCOCIZUMAB:Blood pressure abnormal:0.21), (BOSUTINIB:Blood pressure abnormal:0.03), (BOTULINUM TOXIN TYPE A:Blood pressure abnormal:0.06), (BRINZOLAMIDE\TIMOLOL MALEATE:Blood pressure abnormal:0.42), (BUDESONIDE\FORMOTEROL:Blood pressure abnormal:0.37), (CABOZANTINIB S-MALATE:Blood pressure abnormal:0.3), (CALCIUM CARBONATE\ERGOCALCIFEROL\RETINOL:Blood pressure abnormal:0.83), (CALCIUM CARBONATE\ETIDRONATE DISODIUM:Blood pressure abnormal:0.17), (CALCIUM CHLORIDE\DEXTROSE MONOHYDRATE\MAGNESIUM CHLORIDE\SODIUM CHLORIDE\SODIUM LACTATE:Blood pressure abnormal:0.36), (CALCIUM\VITAMIN D\VITAMIN K5:Blood pressure abnormal:0.2), (CARBASALATE:Blood pressure abnormal:0.02), (CARBIMAZOLE:Blood pressure abnormal:0.6), (CASPOFUNGIN:Blood pressure abnormal:0.11), (CEFEPIME\CEFEPIME HYDROCHLORIDE:Blood pressure abnormal:0.42), (CEFPODOXIME PROXETIL:Blood pressure abnormal:0.32), (CEFTAZIDIME:Blood pressure abnormal:0.29), (CHLORDIAZEPOXIDE\CLIDINIUM:Blood pressure abnormal:0.1), (CICLOPIROX OLAMINE:Blood pressure abnormal:0.08), (CISATRACURIUM:Blood pressure abnormal:0.09), (CLIKSTAR:Blood pressure abnormal:1.18), (CODEINE PHOSPHATE:Blood pressure abnormal:0.45), (CODEINE PHOSPHATE\DICLOFENAC SODIUM:Blood pressure abnormal:0.2), (CORTISONE ACETATE:Blood pressure abnormal:0.62), (CYPROHEPTADINE HYDROCHLORIDE:Blood pressure abnormal:0.32), (DABRAFENIB MESYLATE:Blood pressure abnormal:0.05), (DACLIZUMAB:Blood pressure abnormal:1.88), (DALTEPARIN:Blood pressure abnormal:1.14), (DEFERIPRONE:Blood pressure abnormal:0.13), (DEGARELIX ACETATE:Blood pressure abnormal:0.2), (DEHYDROEPIANDROSTERONE-3-SULFATE SODIUM:Blood pressure abnormal:0.12), (DEXAMETHASONE ACETATE:Blood pressure abnormal:0.4), (DEXMEDETOMIDINE HYDROCHLORIDE:Blood pressure abnormal:0.24), (DIFLUNISAL:Blood pressure abnormal:0.69), (DIMETHYL SULFOXIDE:Blood pressure abnormal:0.01), (DOLASETRON MESYLATE:Blood pressure abnormal:0.98), (ECONAZOLE:Blood pressure abnormal:0.74), (ELBASVIR\GRAZOPREVIR:Blood pressure abnormal:0.02), (ELOTUZUMAB:Blood pressure abnormal:0.69), (EMPAGLIFLOZIN\METFORMIN HYDROCHLORIDE:Blood pressure abnormal:0.53), (ESTRADIOL ACETATE:Blood pressure abnormal:0.18), (ETHINYL ESTRADIOL\NORETHINDRONE ACETATE:Blood pressure abnormal:0.51), (ETHINYL ESTRADIOL\NORGESTREL:Blood pressure abnormal:1), (ETIMICIN SULFATE:Blood pressure abnormal:0.43), (FERRIC HYDROXIDE:Blood pressure abnormal:0.59), (FERROUS GLYCINE SULFATE:Blood pressure abnormal:0.26), (FLAVOXATE HYDROCHLORIDE:Blood pressure abnormal:0), (FLUINDIONE:Blood pressure abnormal:0.65), (FLUNARIZINE HYDROCHLORIDE:Blood pressure abnormal:0.62), (FLUOCINOLONE:Blood pressure abnormal:0.29), (FONDAPARINUX:Blood pressure abnormal:0.41), (FONDAPARINUX SODIUM:Blood pressure abnormal:0.58), (FORMOTEROL FUMARATE\MOMETASONE FUROATE:Blood pressure abnormal:1.36), (GIMERACIL\OTERACIL\TEGAFUR:Blood pressure abnormal:0.12), (GLUTAMINE:Blood pressure abnormal:0.18), (GLYCOPYRRONIUM:Blood pressure abnormal:0.93), (GOLD:Blood pressure abnormal:0.07), (HEPARIN CALCIUM:Blood pressure abnormal:0.09), (HERBALS\MINERALS\VITAMINS:Blood pressure abnormal:0.18), (HYDRALAZINE HYDROCHLORIDE\ISOSORBIDE DINITRATE:Blood pressure abnormal:0.08), (HYDROXOCOBALAMIN:Blood pressure abnormal:0.1), (HYDROXYCITRIC ACID:Blood pressure abnormal:0.17), (IDARUCIZUMAB:Blood pressure abnormal:0.14), (ILOPROST:Blood pressure abnormal:1.43), (IMIGLUCERASE:Blood pressure abnormal:0.27), (IMIPRAMINE PAMOATE:Blood pressure abnormal:0.15), (INTERFERON ALFA-2A:Blood pressure abnormal:0.93), (INTERFERON ALFA-2B:Blood pressure abnormal:0.6), (INTERFERON GAMMA-1B:Blood pressure abnormal:0.4), (IODINE:Blood pressure abnormal:1.14), (IRINOTECAN:Blood pressure abnormal:0.53), (IRON\POLYSACCHARIDES:Blood pressure abnormal:0.83), (ISOFLURANE:Blood pressure abnormal:0.23), (ITRACONAZOLE\SODIUM CHLORIDE:Blood pressure abnormal:0.01), (IXAZOMIB:Blood pressure abnormal:0.98), (LENVATINIB:Blood pressure abnormal:0.27), (LEVOFLOXACIN HYDROCHLORIDE:Blood pressure abnormal:0.2), (LEVOLEUCOVORIN CALCIUM:Blood pressure abnormal:0.01), (LEVOTHYROXINE SODIUM\POTASSIUM IODIDE:Blood pressure abnormal:0.14), (LORMETAZEPAM:Blood pressure abnormal:0.07), (LORNOXICAM:Blood pressure abnormal:0.25), (MECLIZINE MONOHYDROCHLORIDE\NIACIN:Blood pressure abnormal:0.15), (MECLOZINE DIHYDROCHLORIDE:Blood pressure abnormal:0.05), (MENTHOL\ZINC OXIDE:Blood pressure abnormal:0.2), (METHENAMINE MANDELATE:Blood pressure abnormal:0.96), (METRELEPTIN:Blood pressure abnormal:0.64), (MORNIFLUMATE:Blood pressure abnormal:0.1), (NAFTIFINE HYDROCHLORIDE:Blood pressure abnormal:0.02), (NEOMYCIN:Blood pressure abnormal:0.32), (OPIPRAMOL:Blood pressure abnormal:0.45), (OSELTAMIVIR PHOSPHATE:Blood pressure abnormal:0.93), (PAMIDRONATE DISODIUM:Blood pressure abnormal:1.71), (PARATHYROID HORMONE:Blood pressure abnormal:0.79), (PEGINTERFERON ALFA-2B:Blood pressure abnormal:0.08), (PEMETREXED:Blood pressure abnormal:0.49), (PEMETREXED DISODIUM:Blood pressure abnormal:0.58), (PHOSPHOLIPID:Blood pressure abnormal:0.08), (PIMECROLIMUS:Blood pressure abnormal:1.02), (PINAVERIUM BROMIDE:Blood pressure abnormal:0.24), (PINDOLOL:Blood pressure abnormal:0.3), (PIRFENIDONE:Blood pressure abnormal:0.24), (POLYETHYLENE GLYCOL 4000:Blood pressure abnormal:0.35), (PRAMOXINE HYDROCHLORIDE:Blood pressure abnormal:0.56), (PREDNISOLONE SODIUM PHOSPHATE:Blood pressure abnormal:0.88), (PREDNISOLONE\PREDNISOLONE ACETATE\PREDNISOLONE SODIUM SUCCINATE:Blood pressure abnormal:1.1), (PREGNENOLONE:Blood pressure abnormal:0.26), (PROTRIPTYLINE HYDROCHLORIDE:Blood pressure abnormal:1.46), (PRUCALOPRIDE SUCCINATE:Blood pressure abnormal:0.26), (PYRAZINAMIDE:Blood pressure abnormal:0.78), (QUINACRINE:Blood pressure abnormal:0.39), (QUINAGOLIDE HYDROCHLORIDE:Blood pressure abnormal:0.2), (QUINIDINE GLUCONATE:Blood pressure abnormal:0.15), (ROSIGLITAZONE:Blood pressure abnormal:0.14), (SEVELAMER:Blood pressure abnormal:0.11), (SODIUM CITRATE:Blood pressure abnormal:0.8), (SOFOSBUVIR\VELPATASVIR:Blood pressure abnormal:0.55), (SORBITOL:Blood pressure abnormal:0.04), (SUPLATAST TOSILATE:Blood pressure abnormal:0.2), (TAFAMIDIS MEGLUMINE:Blood pressure abnormal:0.13), (TAZOBACTAM:Blood pressure abnormal:0.01), (TELOTRISTAT ETHYL:Blood pressure abnormal:0.78), (TERBUTALINE:Blood pressure abnormal:0.83), (TESAMORELIN:Blood pressure abnormal:0.45), (TESTOSTERONE UNDECANOATE:Blood pressure abnormal:1.7), (THIOCTIC ACID:Blood pressure abnormal:0.74), (TIMOLOL MALEATE\TRAVOPROST:Blood pressure abnormal:1.05), (TIMOLOL\TRAVOPROST:Blood pressure abnormal:0.48), (TINZAPARIN:Blood pressure abnormal:0.27), (TINZAPARIN SODIUM:Blood pressure abnormal:0.71), (TRAMETINIB DIMETHYL SULFOXIDE:Blood pressure abnormal:0.07), (TREPROSTINIL DIOLAMINE:Blood pressure abnormal:0.15), (TRIMIPRAMINE MALEATE:Blood pressure abnormal:0.46), (TRIPTORELIN PAMOATE:Blood pressure abnormal:1.28), (TROSPIUM:Blood pressure abnormal:0.43), (VECURONIUM BROMIDE:Blood pressure abnormal:0.28), (VINORELBINE TARTRATE:Blood pressure abnormal:0.66), (VISMODEGIB:Blood pressure abnormal:0.38), (VITAMIN A PALMITATE:Blood pressure abnormal:0.23), (VORAPAXAR SULFATE:Blood pressure abnormal:0.12), (ACETAMINOPHEN\CODEINE PHOSPHATE:Blood pressure decreased:0.47), (ALBUTEROL SULFATE\IPRATROPIUM BROMIDE:Blood pressure decreased:0.03), (BUMETANIDE:Blood pressure decreased:0.05), (BUPROPION HYDROCHLORIDE:Blood pressure decreased:0.8), (CALCIUM ACETATE:Blood pressure decreased:0.52), (CALCIUM CARBONATE\CHOLECALCIFEROL:Blood pressure decreased:0.19), (CEFOPERAZONE:Blood pressure decreased:0.06), (CEFOTIAM:Blood pressure decreased:0.02), (CLOTRIMAZOLE:Blood pressure decreased:0.04), (DESLANOSIDE:Blood pressure decreased:0.34), (DEXLANSOPRAZOLE:Blood pressure decreased:0.66), (DOXYCYCLINE:Blood pressure decreased:0.09), (ENALAPRIL MALEATE:Blood pressure decreased:0.05), (ESMOLOL HYDROCHLORIDE:Blood pressure decreased:0.53), (ETIMICIN SULFATE:Blood pressure decreased:0.74), (EVEROLIMUS:Blood pressure decreased:0.59), (EVOLOCUMAB:Blood pressure decreased:0.76), (EZETIMIBE:Blood pressure decreased:0.19), (FLECAINIDE:Blood pressure decreased:0.03), (HYDROMORPHONE:Blood pressure decreased:0), (IBRUTINIB:Blood pressure decreased:0.78), (ILOPROST:Blood pressure decreased:0.12), (ISOFLURANE:Blood pressure decreased:0.81), (ISOSORBIDE:Blood pressure decreased:0.11), (LENALIDOMIDE:Blood pressure decreased:0.56), (LERCANIDIPINE:Blood pressure decreased:0.21), (LEUCOGEN:Blood pressure decreased:0.78), (LEVOFLOXACIN HYDROCHLORIDE:Blood pressure decreased:0.55), (LOVASTATIN:Blood pressure decreased:0.43), (NAPROXEN:Blood pressure decreased:0.9), (NEBIVOLOL:Blood pressure decreased:0.01), (NEOSTIGMINE:Blood pressure decreased:0.92), (NILOTINIB:Blood pressure decreased:0.3), (NYSTATIN:Blood pressure decreased:0.23), (OLANZAPINE:Blood pressure decreased:0.13), (POLYETHYLENE GLYCOLS:Blood pressure decreased:0.37), (PRAVASTATIN:Blood pressure decreased:0.59), (PROTAMINE SULFATE:Blood pressure decreased:0.06), (RANOLAZINE:Blood pressure decreased:0.76), (SPIRONOLACTONE:Blood pressure decreased:1.3), (SUFENTANIL:Blood pressure decreased:0.34), (TERIFLUNOMIDE:Blood pressure decreased:0.17), (THIOPENTAL:Blood pressure decreased:0.32), (TRICHLORMETHIAZIDE:Blood pressure decreased:0.02), (VALGANCICLOVIR HYDROCHLORIDE:Blood pressure decreased:0.65), (VARENICLINE TARTRATE:Blood pressure decreased:0.01), (IBRUTINIB:Blood pressure fluctuation:0.16), (NAPROXEN:Blood pressure fluctuation:0.02), (OMEPRAZOLE MAGNESIUM:Blood pressure fluctuation:0.07), (.ALPHA.1-PROTEINASE INHIBITOR HUMAN:Blood pressure increased:0.6), (ACETAMINOPHEN\CAFFEINE:Blood pressure increased:0.25), (ACETAMINOPHEN\DIPHENHYDRAMINE HYDROCHLORIDE:Blood pressure increased:0.04), (ACETAMINOPHEN\OXYCODONE\OXYCODONE HYDROCHLORIDE:Blood pressure increased:1.59), (ACTIVATED CHARCOAL:Blood pressure increased:0.57), (ALBIGLUTIDE:Blood pressure increased:0.31), (ALDESLEUKIN:Blood pressure increased:0.15), (ALEMTUZUMAB:Blood pressure increased:0.35), (AMANTADINE HYDROCHLORIDE:Blood pressure increased:0.48), (AMINO ACIDS\DEXTROSE\ELECTROLYTES NOS:Blood pressure increased:0.07), (AMINOPHYLLINE:Blood pressure increased:0.12), (AMLODIPINE\PERINDOPRIL:Blood pressure increased:0.2), (AMMONIUM CHLORIDE\DEXTROMETHORPHAN\SODIUM CITRATE:Blood pressure increased:0.2), (AMOXICILLIN\CLARITHROMYCIN\ESOMEPRAZOLE:Blood pressure increased:0.06), (AMPICILLIN SODIUM\SULBACTAM SODIUM:Blood pressure increased:0.08), (ANTIHISTAMINES NOS:Blood pressure increased:0.34), (AVANAFIL:Blood pressure increased:0.16), (BACITRACIN:Blood pressure increased:0.15), (BELATACEPT:Blood pressure increased:0.21), (BETAMETHASONE DIPROPIONATE:Blood pressure increased:0.46), (BETAMETHASONE DIPROPIONATE\CALCIPOTRIENE\CALCIPOTRIENE HYDRATE:Blood pressure increased:0.28), (BETAMETHASONE\CALCIPOTRIENE:Blood pressure increased:0.39), (BIPERIDEN HYDROCHLORIDE:Blood pressure increased:0.07), (BLESELUMAB:Blood pressure increased:0.04), (BLINATUMOMAB:Blood pressure increased:0.21), (BUPRENORPHINE HYDROCHLORIDE:Blood pressure increased:1.26), (CALCITONIN:Blood pressure increased:0.57), (CALCIUM\MAGNESIUM:Blood pressure increased:0.59), (CARBIMAZOLE:Blood pressure increased:0.38), (CASANTHRANOL\DOCUSATE SODIUM:Blood pressure increased:0.38), (CEFAZOLIN:Blood pressure increased:0.95), (CEFEPIME HYDROCHLORIDE:Blood pressure increased:0.82), (CEFPODOXIME PROXETIL:Blood pressure increased:0.09), (CETIRIZINE HYDROCHLORIDE\PSEUDOEPHEDRINE HYDROCHLORIDE:Blood pressure increased:0.57), (CETUXIMAB:Blood pressure increased:0.75), (CHLORDIAZEPOXIDE:Blood pressure increased:0.04), (CHLORHEXIDINE GLUCONATE:Blood pressure increased:0.28), (CHLORPHENIRAMINE\HYDROCODONE:Blood pressure increased:0.33), (CISATRACURIUM:Blood pressure increased:0.01), (CLIKSTAR:Blood pressure increased:0.22), (CLOBAZAM:Blood pressure increased:0.31), (CLOTRIMAZOLE:Blood pressure increased:1.76), (CODEINE PHOSPHATE\GUAIFENESIN:Blood pressure increased:0.25), (CORTISONE ACETATE:Blood pressure increased:0.05), (CYPROHEPTADINE:Blood pressure increased:0.86), (DABIGATRAN:Blood pressure increased:1.44), (DACLIZUMAB:Blood pressure increased:1.47), (DAPAGLIFLOZIN PROPANEDIOL\METFORMIN HYDROCHLORIDE:Blood pressure increased:0.48), (DARATUMUMAB:Blood pressure increased:0.02), (DEFLAZACORT:Blood pressure increased:1.26), (DEGARELIX ACETATE:Blood pressure increased:0.03), (DESONIDE:Blood pressure increased:0.72), (DEXAMETHASONE ACETATE:Blood pressure increased:0.61), (DEXMEDETOMIDINE HYDROCHLORIDE:Blood pressure increased:0.44), (DIGITOXIN:Blood pressure increased:0.86), (DOLASETRON MESYLATE:Blood pressure increased:0.3), (DORZOLAMIDE:Blood pressure increased:0.74), (DOXYLAMINE SUCCINATE:Blood pressure increased:0.02), (ECONAZOLE:Blood pressure increased:0.5), (ELOTUZUMAB:Blood pressure increased:0.83), (EMPAGLIFLOZIN\METFORMIN HYDROCHLORIDE:Blood pressure increased:0.01), (ERIBULIN:Blood pressure increased:0.54), (ERLOTINIB:Blood pressure increased:0.02), (ESTRADIOL\NORETHINDRONE ACETATE:Blood pressure increased:0), (ETOPOSIDE:Blood pressure increased:0.05), (ETORICOXIB:Blood pressure increased:1.21), (FENOTEROL HYDROBROMIDE:Blood pressure increased:0.38), (FERROUS SULFATE\GLYCINE:Blood pressure increased:0.21), (FLUINDIONE:Blood pressure increased:0.83), (FLURAZEPAM\FLURAZEPAM HYDROCHLORIDE:Blood pressure increased:0.09), (FLUVOXAMINE MALEATE:Blood pressure increased:0.69), (FOLATE SODIUM:Blood pressure increased:0.5), (FONDAPARINUX SODIUM:Blood pressure increased:0.25), (FORMOTEROL FUMARATE\MOMETASONE FUROATE:Blood pressure increased:0.79), (GIMERACIL\OTERACIL\TEGAFUR:Blood pressure increased:0.36), (GLIPIZIDE\METFORMIN HYDROCHLORIDE:Blood pressure increased:0.28), (GLYCOPYRROLATE\INDACATEROL MALEATE:Blood pressure increased:0.43), (GLYCOPYRRONIUM:Blood pressure increased:0.58), (HERBALS\SAW PALMETTO:Blood pressure increased:0.03), (HYALURONATE SODIUM:Blood pressure increased:0.15), (ICOSAPENT ETHYL:Blood pressure increased:0.35), (ILOPROST:Blood pressure increased:1.05), (INSULIN GLULISINE:Blood pressure increased:1.07), (INSULIN PORK\ANSULIN PURIFIED PORK:Blood pressure increased:0.12), (INTERFERON ALFA-2A:Blood pressure increased:0.66), (INTERFERON ALFA-2B:Blood pressure increased:0.77), (IOPAMIDOL:Blood pressure increased:0.33), (IRINOTECAN HYDROCHLORIDE:Blood pressure increased:0.73), (IVABRADINE HYDROCHLORIDE:Blood pressure increased:0.11), (IXAZOMIB:Blood pressure increased:0.45), (LACTASE:Blood pressure increased:0.66), (LANREOTIDE:Blood pressure increased:0.36), (LAPATINIB DITOSYLATE:Blood pressure increased:0.71), (LEVOFLOXACIN HYDROCHLORIDE:Blood pressure increased:1.12), (LEVOMEFOLATE CALCIUM\METHYLCOBALAMIN\PYRIDOXAL PHOSPHATE ANHYDROUS:Blood pressure increased:0.22), (LIDOCAINE\PRILOCAINE:Blood pressure increased:0.97), (LUTETIUM LU-177:Blood pressure increased:0.35), (MEGESTROL:Blood pressure increased:0.15), (MEPOLIZUMAB:Blood pressure increased:0.51), (MESNA:Blood pressure increased:0.11), (METFORMIN HYDROCHLORIDE\ROSIGLITAZONE MALEATE:Blood pressure increased:0.49), (METHENAMINE HIPPURATE:Blood pressure increased:1.52), (METHENOLONE ENANTHATE:Blood pressure increased:0.14), (METILDIGOXIN:Blood pressure increased:0.18), (MICONAZOLE NITRATE:Blood pressure increased:0.42), (MINOCYCLINE HYDROCHLORIDE:Blood pressure increased:0.78), (MOEXIPRIL HYDROCHLORIDE:Blood pressure increased:0.52), (NALOXONE:Blood pressure increased:0.22), (NARATRIPTAN HYDROCHLORIDE:Blood pressure increased:0.25), (NATEGLINIDE:Blood pressure increased:1.36), (NORETHINDRONE:Blood pressure increased:0.65), (OLOPATADINE:Blood pressure increased:0.25), (ONABOTULINUMTOXINA:Blood pressure increased:0.64), (OSELTAMIVIR:Blood pressure increased:0.46), (OXYBUTYNIN\OXYBUTYNIN CHLORIDE:Blood pressure increased:0.49), (PAMIDRONATE DISODIUM:Blood pressure increased:0.94), (PANITUMUMAB:Blood pressure increased:0.08), (PARATHYROID HORMONE:Blood pressure increased:0.44), (PEGINTERFERON ALFA-2B:Blood pressure increased:0.63), (PENICILLIN G:Blood pressure increased:0.3), (PENTOSTATIN:Blood pressure increased:0.18), (PILOCARPINE:Blood pressure increased:0.5), (PIMOBENDAN:Blood pressure increased:0.07), (PRAMIPEXOLE:Blood pressure increased:1.66), (PRAMLINTIDE ACETATE:Blood pressure increased:0.06), (PREDNISOLONE ACETATE:Blood pressure increased:1.58), (PREDNISOLONE SODIUM PHOSPHATE:Blood pressure increased:0.06), (PYRAZINAMIDE:Blood pressure increased:0.76), (PYRIDOSTIGMINE BROMIDE:Blood pressure increased:0.48), (RETINOL:Blood pressure increased:1.04), (RIZATRIPTAN:Blood pressure increased:0.35), (SALMETEROL XINAFOATE:Blood pressure increased:0.74), (SARPOGRELATE:Blood pressure increased:0.09), (SILVER SULFADIAZINE:Blood pressure increased:0.02), (SIPULEUCEL-T:Blood pressure increased:0.36), (SODIUM CITRATE:Blood pressure increased:0.46), (STARCH, WHEAT:Blood pressure increased:0.09), (SUMATRIPTAN SUCCINATE:Blood pressure increased:0.8), (TAZOBACTAM:Blood pressure increased:0.15), (TERBUTALINE:Blood pressure increased:0.42), (TETRACYCLINE:Blood pressure increased:1.44), (THEOPHYLLINE:Blood pressure increased:0.1), (THIOCTIC ACID:Blood pressure increased:0.21), (TINZAPARIN:Blood pressure increased:0.78), (TINZAPARIN SODIUM:Blood pressure increased:0.64), (TIPIRACIL HYDROCHLORIDE\TRIFLURIDINE:Blood pressure increased:0.04), (TRIAMCINOLONE:Blood pressure increased:0.76), (TRIPTORELIN PAMOATE:Blood pressure increased:0.38), (UMECLIDINIUM BROMIDE:Blood pressure increased:0.63), (VINORELBINE TARTRATE:Blood pressure increased:0.78), (YEAST:Blood pressure increased:0.15), (ZALEPLON:Blood pressure increased:0), (ZILEUTON:Blood pressure increased:0.42), (ZONISAMIDE:Blood pressure increased:0.5), (ACETAMINOPHEN\OXYCODONE\OXYCODONE HYDROCHLORIDE:Blood pressure management:0.56), (ACETAZOLAMIDE:Blood pressure management:0.75), (AGALSIDASE BETA:Blood pressure management:0.35), (ALBIGLUTIDE:Blood pressure management:0.16), (ALCOHOL:Blood pressure management:0.07), (ALENDRONIC ACID:Blood pressure management:0.25), (ALISKIREN HEMIFUMARATE:Blood pressure management:0.24), (AMISULPRIDE:Blood pressure management:0.16),(ARMODAFINIL:Blood pressure management:0.97), (ASPIRIN LYSINE:Blood pressure management:0.22), (ATRACURIUM:Blood pressure management:0.94), (BUPIVACAINE\EPINEPHRINE:Blood pressure management:0.11), (CALCITONIN SALMON:Blood pressure management:0.11), (CALCIUM ACETATE:Blood pressure management:1.31), (CALCIUM CARBONATE\VITAMIN D:Blood pressure management:0.05), (CANAGLIFLOZIN\METFORMIN HYDROCHLORIDE:Blood pressure management:0.69), (CEFUROXIME:Blood pressure management:0.16), (CERTOLIZUMAB PEGOL:Blood pressure management:1.06), (CIMETIDINE:Blood pressure management:0.65), (CINACALCET HYDROCHLORIDE:Blood pressure management:0.29), (CLARITHROMYCIN:Blood pressure management:0.17), (CLOMIPRAMINE HYDROCHLORIDE:Blood pressure management:0), (CLOTRIMAZOLE:Blood pressure management:0.99), (CLOZAPINE:Blood pressure management:0.85), (DACLIZUMAB:Blood pressure management:0.2), (DANAZOL:Blood pressure management:0.01), (DASATINIB:Blood pressure management:0.91), (DESLORATADINE:Blood pressure management:0.57), (DEXAMETHASONE ACETATE:Blood pressure management:0.16), (DIGITOXIN:Blood pressure management:0.4), (DIPYRIDAMOLE:Blood pressure management:0.41), (DOXEPIN HYDROCHLORIDE:Blood pressure management:0.23), (DRONABINOL:Blood pressure management:0.5), (EDOXABAN TOSYLATE:Blood pressure management:1.02), (EMPAGLIFLOZIN\LINAGLIPTIN:Blood pressure management:0.95), (EMPAGLIFLOZIN\METFORMIN HYDROCHLORIDE:Blood pressure management:0.12), (ESZOPICLONE:Blood pressure management:0.82), (FERROUS SULFATE\GLYCINE:Blood pressure management:0.02), (FISH OIL\OMEGA-3 FATTY ACIDS:Blood pressure management:0.22), (FLUDROCORTISONE ACETATE:Blood pressure management:0.33), (FLUNITRAZEPAM:Blood pressure management:0.01), (FLUTICASONE\FLUTICASONE PROPIONATE:Blood pressure management:0.67), (FORMOTEROL FUMARATE:Blood pressure management:0.26), (GLYCOPYRRONIUM\INDACATEROL:Blood pressure management:0.43), (HERBALS\TURMERIC:Blood pressure management:0.21), (HYALURONATE SODIUM:Blood pressure management:0.01), (ILOPROST:Blood pressure management:1.02), (IMIPRAMINE HYDROCHLORIDE:Blood pressure management:0.44), (INDAPAMIDE\PERINDOPRIL:Blood pressure management:0.8), (INFLUENZA VIRUS VACCINE:Blood pressure management:0.61), (IRON SUCROSE:Blood pressure management:0.08), (KETAMINE:Blood pressure management:0.06), (KETOPROFEN:Blood pressure management:0.05), (LERCANIDIPINE HYDROCHLORIDE:Blood pressure management:0.71), (LIDOCAINE\PRILOCAINE:Blood pressure management:0.06), (LIOTHYRONINE SODIUM:Blood pressure management:0.18), (LOMITAPIDE MESYLATE:Blood pressure management:0.3), (LOPERAMIDE:Blood pressure management:0.12), (MEMANTINE HYDROCHLORIDE:Blood pressure management:0.1), (METFORMIN HYDROCHLORIDE\ROSIGLITAZONE MALEATE:Blood pressure management:0.23), (METFORMIN HYDROCHLORIDE\VILDAGLIPTIN:Blood pressure management:0.25), (METHENAMINE HIPPURATE:Blood pressure management:0.62), (METHIMAZOLE:Blood pressure management:0.44), (METHYLPHENIDATE HYDROCHLORIDE:Blood pressure management:0.63), (MOXIFLOXACIN:Blood pressure management:0.06), (NALOXONE HYDROCHLORIDE\OXYCODONE HYDROCHLORIDE:Blood pressure management:0.61), (NALOXONE HYDROCHLORIDE\TILIDINE HYDROCHLORIDE:Blood pressure management:0.11), (NATEGLINIDE:Blood pressure management:0.4), (NEOSTIGMINE:Blood pressure management:1.36), (NILOTINIB:Blood pressure management:1.19), (OSELTAMIVIR:Blood pressure management:0.02), (PANTOPRAZOLE MAGNESIUM:Blood pressure management:1), (PEGINTERFERON BETA-1A:Blood pressure management:0.3), (PEMETREXED DISODIUM:Blood pressure management:0.23), (PENICILLIN:Blood pressure management:0.7), (PHENOBARBITAL:Blood pressure management:0.71), (PHYTONADIONE:Blood pressure management:0.1), (PIROXICAM:Blood pressure management:0.07), (PLANTAGO SEED:Blood pressure management:0.31), (POLYETHYLENE GLYCOLS:Blood pressure management:1.31), (PREDNISOLONE ACETATE:Blood pressure management:0.19), (PYRIDOSTIGMINE BROMIDE:Blood pressure management:0.2), (PYRIDOXINE:Blood pressure management:0.06), (RABEPRAZOLE:Blood pressure management:0.68), (RALOXIFENE HYDROCHLORIDE:Blood pressure management:0.71), (RISEDRONATE SODIUM:Blood pressure management:0.61), (RIVASTIGMINE:Blood pressure management:0.04), (RIVASTIGMINE TARTRATE:Blood pressure management:0.39), (RIZATRIPTAN BENZOATE:Blood pressure management:0.51), (SECUKINUMAB:Blood pressure management:0.3), (SODIUM CITRATE:Blood pressure management:0.25), (SUFENTANIL CITRATE:Blood pressure management:0.15), (SUGAMMADEX SODIUM:Blood pressure management:0.16), (SUMATRIPTAN\SUMATRIPTAN SUCCINATE:Blood pressure management:0.87), (TERBINAFINE HYDROCHLORIDE:Blood pressure management:0.19), (TERBUTALINE:Blood pressure management:0.27), (TERBUTALINE SULFATE:Blood pressure management:0.16), (THIAMYLAL SODIUM:Blood pressure management:0.48), (THYROID, PORCINE:Blood pressure management:0.08), (TOCILIZUMAB:Blood pressure management:0.25), (TRIAMCINOLONE:Blood pressure management:0.03), (TROSPIUM CHLORIDE:Blood pressure management:0.13), (UMECLIDINIUM BROMIDE\VILANTEROL TRIFENATATE:Blood pressure management:0.88), (VILAZODONE HYDROCHLORIDE:Blood pressure management:0.32), (VITAMIN B:Blood pressure management:0.93), (ZINC:Blood pressure management:0.14), (ABOBOTULINUMTOXINA:Blood pressure measurement:0.39), (ACEBUTOLOL HYDROCHLORIDE:Blood pressure measurement:0.02), (ACETAMINOPHEN\BUTALBITAL\CAFFEINE\CODEINE PHOSPHATE:Blood pressure measurement:0.12), (ACETAMINOPHEN\CAFFEINE\CARISOPRODOL\DICLOFENAC SODIUM:Blood pressure measurement:0.12), (ACETAMINOPHEN\CHLORZOXAZONE:Blood pressure measurement:0.02), (ACETAMINOPHEN\METHOCARBAMOL:Blood pressure measurement:0.4), (ALBUTEROL\IPRATROPIUM BROMIDE:Blood pressure measurement:0.01), (ALUMINUM HYDROXIDE\MAGNESIUM HYDROXIDE\SIMETHICONE:Blood pressure measurement:0.11), (AMIKACIN:Blood pressure measurement:0.29), (AMINO ACIDS\DEXTROSE\ELECTROLYTES NOS:Blood pressure measurement:1.09), (AMINOPHYLLINE:Blood pressure measurement:0.11), (AMMONIUM CHLORIDE\DEXTROMETHORPHAN\SODIUM CITRATE:Blood pressure measurement:0.8), (AMOXICILLIN\CLARITHROMYCIN\ESOMEPRAZOLE:Blood pressure measurement:0.49), (AMPICILLIN SODIUM\SULBACTAM SODIUM:Blood pressure measurement:0.3), (ANTIHISTAMINES NOS:Blood pressure measurement:1.03), (ASCORBIC ACID\BIOTIN\CALCIUM PANTOTHENATE\CYANOCOBALAMIN\FOLIC ACID\NIACINAMIDE\PYRIDOXINE HYDROCHLORIDE\RIBOFLAVIN\THIAMINE MONONITRATE:Blood pressure measurement:0.2), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Blood pressure measurement:0.12), (ASIAN GINSENG:Blood pressure measurement:0.21), (ASPIRIN\CLOPIDOGREL:Blood pressure measurement:0.06), (BENAZEPRIL\BENAZEPRIL HYDROCHLORIDE:Blood pressure measurement:2.2), (BENZOCAINE:Blood pressure measurement:0.13), (BEPOTASTINE BESILATE:Blood pressure measurement:0.04), (BETAMETHASONE\CALCIPOTRIENE:Blood pressure measurement:0.88), (BIMATOPROST\TIMOLOL:Blood pressure measurement:0.12), (BISMUTH SUBSALICYLATE\CALCIUM CARBONATE:Blood pressure measurement:0.3), (BOCOCIZUMAB:Blood pressure measurement:0.15), (BOTULINUM TOXIN TYPE A:Blood pressure measurement:0.02), (BROMFENAC SODIUM:Blood pressure measurement:0.27), (BROTIZOLAM:Blood pressure measurement:0.32), (BUPRENORPHINE\NALOXONE:Blood pressure measurement:0.36), (CABOZANTINIB S-MALATE:Blood pressure measurement:0.34), (CALCITONIN:Blood pressure measurement:1.1), (CALCIUM CARBONATE\ERGOCALCIFEROL:Blood pressure measurement:0.28), (CALCIUM PANTOTHENATE\CYANOCOBALAMIN\NIACINAMIDE\PYRIDOXINE HYDROCHLORIDE\RIBOFLAVIN\THIAMINE MONONITRATE:Blood pressure measurement:0.01), (CAMPHOR\DIPHENHYDRAMINE\ZINC OXIDE:Blood pressure measurement:0), (CARBIMAZOLE:Blood pressure measurement:0.34), (CARTEOLOL HYDROCHLORIDE:Blood pressure measurement:0.08), (CEFAZOLIN SODIUM:Blood pressure measurement:0.54), (CEFEPIME\CEFEPIME HYDROCHLORIDE:Blood pressure measurement:0.1), (CEFOTAXIME SODIUM:Blood pressure measurement:0.67), (CERITINIB:Blood pressure measurement:0.26), (CERIVASTATIN SODIUM:Blood pressure measurement:0.44), (CHLORPHENIRAMINE\HYDROCODONE:Blood pressure measurement:1.06), (CILASTATIN SODIUM\IMIPENEM:Blood pressure measurement:1.2), (CISATRACURIUM:Blood pressure measurement:0.26), (COLISTIMETHATE SODIUM:Blood pressure measurement:0.02), (CYPROHEPTADINE HYDROCHLORIDE:Blood pressure measurement:0.29), (DACLIZUMAB:Blood pressure measurement:2.01), (DANAZOL:Blood pressure measurement:1.57), (DANTROLENE SODIUM:Blood pressure measurement:0.38), (DESFLURANE:Blood pressure measurement:0.9), (DESMOPRESSIN:Blood pressure measurement:0.95), (DEXAMETHASONE ACETATE:Blood pressure measurement:0.28), (DEXAMETHASONE\TOBRAMYCIN:Blood pressure measurement:0.59), (DEXMEDETOMIDINE HYDROCHLORIDE:Blood pressure measurement:0.65), (DIFLUNISAL:Blood pressure measurement:0.73), (DIMETHICONE\SILICON DIOXIDE\SIMETHICONE:Blood pressure measurement:0.17), (DOBUTAMINE HYDROCHLORIDE:Blood pressure measurement:0.21), (DOLASETRON MESYLATE:Blood pressure measurement:0.79), (DROSPIRENONE\ETHINYL ESTRADIOL:Blood pressure measurement:0.75), (ECONAZOLE:Blood pressure measurement:0.87), (ELBASVIR\GRAZOPREVIR:Blood pressure measurement:0.27), (ENSULIZOLE\EUCERIN\OCTINOXATE\OCTISALATE\TITANIUM DIOXIDE\ZINC OXIDE:Blood pressure measurement:0.08), (ESTRADIOL HEMIHYDRATE:Blood pressure measurement:0.01), (ESTRIOL:Blood pressure measurement:0.63), (ETHAMBUTOL HYDROCHLORIDE:Blood pressure measurement:0.01), (ETHINYL ESTRADIOL\NORGESTREL:Blood pressure measurement:0.78), (ETORICOXIB:Blood pressure measurement:1.92), (FENOTEROL HYDROBROMIDE\IPRATROPIUM BROMIDE:Blood pressure measurement:0.5), (FERRIC HYDROXIDE:Blood pressure measurement:0.27), (FERRIC POLYSACCHARIDE COMPLEX:Blood pressure measurement:0.14), (FERROUS SULFATE\GLYCINE:Blood pressure measurement:0.34), (FLUINDIONE:Blood pressure measurement:0.41), (FLUNARIZINE HYDROCHLORIDE:Blood pressure measurement:0.25), (FONDAPARINUX:Blood pressure measurement:0.12), (FORMOTEROL:Blood pressure measurement:1.25), (FROVATRIPTAN SUCCINATE:Blood pressure measurement:0.8), (GEFITINIB:Blood pressure measurement:0.15), (GIMERACIL\OTERACIL\TEGAFUR:Blood pressure measurement:0.11), (GLUTAMINE:Blood pressure measurement:0.04), (GLYCOPYRROLATE\INDACATEROL MALEATE:Blood pressure measurement:0.79), (GOLD:Blood pressure measurement:0.2), (GRANISETRON HYDROCHLORIDE:Blood pressure measurement:0.08), (HORSE CHESTNUT:Blood pressure measurement:0.24), (HYDROXYPROPYL CELLULOSE:Blood pressure measurement:0.2), (HYOSCYAMINE SULFATE\METHENAMINE\METHYLENE BLUE\PHENYL SALICYLATE\SODIUM PHOSPHATE, MONOBASIC, MONOHYDRATE:Blood pressure measurement:0.52), (INDAPAMIDE\PERINDOPRIL:Blood pressure measurement:1.76), (INSULIN PORK\INSULIN PURIFIED PORK:Blood pressure measurement:0.67), (INTERFERON:Blood pressure measurement:1.3), (INTERFERON ALFA-2A:Blood pressure measurement:0.96), (INTERFERON GAMMA-1B:Blood pressure measurement:0.41), (IRINOTECAN HYDROCHLORIDE:Blood pressure measurement:0.97), (IRON\POLYSACCHARIDES:Blood pressure measurement:0.26), (IVERMECTIN:Blood pressure measurement:0.22), (LACTASE:Blood pressure measurement:1.44), (LACTOBACILLUS ACIDOPHILUS\LACTOBACILLUS DELBRUECKII SUBSP. BULGARICUS:Blood pressure measurement:0.15), (LACTOBACILLUS RHAMNOSUS:Blood pressure measurement:1.3), (LANREOTIDE:Blood pressure measurement:0.71), (LATANOPROST\TIMOLOL:Blood pressure measurement:0.25), (LENVATINIB:Blood pressure measurement:0.18), (LEVOLEUCOVORIN CALCIUM:Blood pressure measurement:0.13), (LUTETIUM LU-177:Blood pressure measurement:0.68), (MEPOLIZUMAB:Blood pressure measurement:1.02), (METHENAMINE MANDELATE:Blood pressure measurement:0.46), (METRELEPTIN:Blood pressure measurement:0.6), (MILRINONE:Blood pressure measurement:0.53), (MOEXIPRIL HYDROCHLORIDE:Blood pressure measurement:1.2), (NADROPARIN CALCIUM:Blood pressure measurement:1.01), (NALOXONE:Blood pressure measurement:0.68), (NALOXONE HYDROCHLORIDE\TILIDINE HYDROCHLORIDE:Blood pressure measurement:0.62), (NAPHAZOLINE HYDROCHLORIDE:Blood pressure measurement:0.2), (NAPROXEN SODIUM\SUMATRIPTAN SUCCINATE:Blood pressure measurement:0.26), (NARATRIPTAN HYDROCHLORIDE:Blood pressure measurement:0.89), (NEOMYCIN:Blood pressure measurement:0.32), (NEOSTIGMINE:Blood pressure measurement:1.02), (PAPAYA:Blood pressure measurement:0.22), (PARATHYROID HORMONE:Blood pressure measurement:0.4), (PASIREOTIDE:Blood pressure measurement:1.02), (PEGINTERFERON BETA-1A:Blood pressure measurement:1.98), (PEMETREXED:Blood pressure measurement:0.73), (PENICILLIN V:Blood pressure measurement:0.88), (PENTOSTATIN:Blood pressure measurement:0.26), (PERMETHRIN:Blood pressure measurement:0.1), (PHENPROCOUMON:Blood pressure measurement:0.8), (POLYETHYLENE GLYCOL 3350\POTASSIUM CHLORIDE\SODIUM BICARBONATE\SODIUM CHLORIDE\SODIUM SULFATE ANHYDROUS:Blood pressure measurement:0.55), (POLYETHYLENE GLYCOL 400:Blood pressure measurement:0.52), (POVIDONE:Blood pressure measurement:0.04), (PREDNISOLONE\PREDNISOLONE ACETATE\PREDNISOLONE SODIUM SUCCINATE:Blood pressure measurement:0.66), (QUINACRINE:Blood pressure measurement:0.25), (QUINIDINE GLUCONATE:Blood pressure measurement:0.04), (RADIUM RA-223 DICHLORIDE:Blood pressure measurement:0.05), (RALTEGRAVIR POTASSIUM:Blood pressure measurement:0.69), (REGORAFENIB:Blood pressure measurement:1.05), (ROSIGLITAZONE:Blood pressure measurement:0.07), (RUCAPARIB CAMSYLATE:Blood pressure measurement:0.44), (SEVELAMER:Blood pressure measurement:0), (SIBUTRAMINE HYDROCHLORIDE:Blood pressure measurement:0.36), (ST. JOHN'S WORT:Blood pressure measurement:0.21), (SUFENTANIL:Blood pressure measurement:0.17), (TAFAMIDIS MEGLUMINE:Blood pressure measurement:0.03), (TELOTRISTAT ETHYL:Blood pressure measurement:0.91), (TERBUTALINE:Blood pressure measurement:0.77), (THEOPHYLLINE:Blood pressure measurement:0.23), (TIBOLONE:Blood pressure measurement:0.31), (TIMOLOL\TRAVOPROST:Blood pressure measurement:0.31), (TINZAPARIN SODIUM:Blood pressure measurement:0.5), (TRIMIPRAMINE MALEATE:Blood pressure measurement:0.34), (VALDECOXIB:Blood pressure measurement:0.45), (VINORELBINE TARTRATE:Blood pressure measurement:0.79), (VORAPAXAR SULFATE:Blood pressure measurement:0.04), (ZILEUTON:Blood pressure measurement:0.9), (RAMIPRIL:Blood pressure systolic increased:0.12), (WARFARIN SODIUM:Blood pressure systolic increased:0.1), (ACARBOSE:Blood testosterone decreased:0.37), (ALENDRONIC ACID:Blood testosterone decreased:0.07), (ALFACALCIDOL:Blood testosterone decreased:0.14), (AMMONIUM LACTATE:Blood testosterone decreased:0.23), (ARIPIPRAZOLE LAUROXIL:Blood testosterone decreased:0.2), (ASPIRIN\DIHYDROXYALUMINUM AMINOACETATE ANHYDROUS\MAGNESIUM CARBONATE:Blood testosterone decreased:0.16), (BRIMONIDINE TARTRATE:Blood testosterone decreased:0.31), (BROMAZEPAM:Blood testosterone decreased:0.07), (BUDESONIDE\FORMOTEROL FUMARATE:Blood testosterone decreased:0.42), (BUPRENORPHINE HYDROCHLORIDE:Blood testosterone decreased:0.72), (CALCITONIN SALMON:Blood testosterone decreased:0.16), (CALCIUM CARBONATE\CHOLECALCIFEROL:Blood testosterone decreased:0.97), (CALCIUM CARBONATE\VITAMIN D:Blood testosterone decreased:0.12), (CALCIUM\MAGNESIUM:Blood testosterone decreased:0.38), (CALCIUM\MAGNESIUM\ZINC:Blood testosterone decreased:0.65), (CANAGLIFLOZIN\METFORMIN HYDROCHLORIDE:Blood testosterone decreased:0.72), (CANAKINUMAB:Blood testosterone decreased:0.11), (CANDESARTAN:Blood testosterone decreased:1.36), (CAPTOPRIL:Blood testosterone decreased:0.89), (CARFILZOMIB:Blood testosterone decreased:0.49), (CARVEDILOL PHOSPHATE:Blood testosterone decreased:1.7), (CEFTRIAXONE SODIUM:Blood testosterone decreased:0.03), (CHLORDIAZEPOXIDE HYDROCHLORIDE\CLIDINIUM BROMIDE:Blood testosterone decreased:0.08), (CINACALCET HYDROCHLORIDE:Blood testosterone decreased:0.38), (CIPROFIBRATE:Blood testosterone decreased:0.23), (CLIKSTAR:Blood testosterone decreased:0.01), (CLINDAMYCIN PHOSPHATE:Blood testosterone decreased:0.24), (CORTICOTROPIN:Blood testosterone decreased:0.52), (DABIGATRAN:Blood testosterone decreased:0.4), (DACLIZUMAB:Blood testosterone decreased:0.83), (DASATINIB:Blood testosterone decreased:1.05), (DIMENHYDRINATE:Blood testosterone decreased:0.18), (DIMETHYL SULFOXIDE:Blood testosterone decreased:0.03), (DORZOLAMIDE HYDROCHLORIDE:Blood testosterone decreased:0.15), (EMPAGLIFLOZIN\LINAGLIPTIN:Blood testosterone decreased:0.89), (ESTROGENS, CONJUGATED\MEDROXYPROGESTERONE ACETATE:Blood testosterone decreased:0.77), (ESTRONE\SUS SCROFA OVARY:Blood testosterone decreased:0.2), (FLUTICASONE FUROATE\SALMETEROL XINAFOATE:Blood testosterone decreased:0.26), (FUSIDIC ACID:Blood testosterone decreased:0.01), (GLICLAZIDE:Blood testosterone decreased:0.77), (GLIMEPIRIDE\ROSIGLITAZONE MALEATE:Blood testosterone decreased:0.16), (GLYCOPYRRONIUM\INDACATEROL:Blood testosterone decreased:0.23), (GUANFACINE:Blood testosterone decreased:0.39), (HALOPERIDOL:Blood testosterone decreased:0.17), (HYDROCODONE BITARTRATE\IBUPROFEN:Blood testosterone decreased:0.41), (IBRUTINIB:Blood testosterone decreased:0.54), (ILOPROST:Blood testosterone decreased:0.12), (IMIPRAMINE:Blood testosterone decreased:0.01), (INSULIN DEGLUDEC:Blood testosterone decreased:0.95), (KETOROLAC\KETOROLAC TROMETHAMINE:Blood testosterone decreased:0.33), (LABETALOL HYDROCHLORIDE:Blood testosterone decreased:0.01), (LEDIPASVIR\SOFOSBUVIR:Blood testosterone decreased:0.62), (LERCANIDIPINE:Blood testosterone decreased:0.28), (LERCANIDIPINE HYDROCHLORIDE:Blood testosterone decreased:0.17), (LIDOCAINE\PRILOCAINE:Blood testosterone decreased:0.52), (LOMITAPIDE MESYLATE:Blood testosterone decreased:0.68), (MEDROXYPROGESTERONE ACETATE:Blood testosterone decreased:0.08), (METHENAMINE HIPPURATE:Blood testosterone decreased:0.92), (METHIMAZOLE:Blood testosterone decreased:0.25), (METHYLPREDNISOLONE ACETATE:Blood testosterone decreased:0.94), (NALOXONE HYDROCHLORIDE\OXYCODONE HYDROCHLORIDE:Blood testosterone decreased:0.15), (NATEGLINIDE:Blood testosterone decreased:0.68), (NILOTINIB:Blood testosterone decreased:0.68), (NITROFURANTOIN:Blood testosterone decreased:0.78), (NITROFURANTOIN\NITROFURANTOIN MONOHYDRATE:Blood testosterone decreased:0.36), (OLODATEROL HYDROCHLORIDE\TIOTROPIUM BROMIDE MONOHYDRATE:Blood testosterone decreased:0.47), (OXYBUTYNIN\OXYBUTYNIN CHLORIDE:Blood testosterone decreased:0.2), (PALIPERIDONE PALMITATE:Blood testosterone decreased:0.15), (PANCRELIPASE:Blood testosterone decreased:0.15), (PANTOPRAZOLE MAGNESIUM:Blood testosterone decreased:0.96), (PARATHYROID HORMONE:Blood testosterone decreased:0.05), (PATIROMER:Blood testosterone decreased:0.15), (PAZOPANIB HYDROCHLORIDE:Blood testosterone decreased:0.14), (PEGFILGRASTIM:Blood testosterone decreased:0.09), (PEGINTERFERON BETA-1A:Blood testosterone decreased:0.87), (PENICILLIN:Blood testosterone decreased:1.19), (PENTOSAN POLYSULFATE SODIUM:Blood testosterone decreased:0.44), (PIROXICAM:Blood testosterone decreased:0.28), (PONATINIB:Blood testosterone decreased:1.08), (PROPAFENONE HYDROCHLORIDE:Blood testosterone decreased:0.61), (PROTRIPTYLINE HYDROCHLORIDE:Blood testosterone decreased:0.14), (RALOXIFENE HYDROCHLORIDE:Blood testosterone decreased:1.29), (RISEDRONATE SODIUM:Blood testosterone decreased:0.67), (RIVASTIGMINE:Blood testosterone decreased:0.2), (ROFLUMILAST:Blood testosterone decreased:0.66), (SACCHAROMYCES CEREVISIAE:Blood testosterone decreased:0.18), (SACUBITRIL\VALSARTAN:Blood testosterone decreased:0.64), (SECUKINUMAB:Blood testosterone decreased:1.19), (SUNITINIB MALATE:Blood testosterone decreased:0.25), (TERBUTALINE SULFATE:Blood testosterone decreased:0.76), (TERIFLUNOMIDE:Blood testosterone decreased:0.79), (TETRACYCLINE:Blood testosterone decreased:1.42), (TOCILIZUMAB:Blood testosterone decreased:0.25), (TOLTERODINE:Blood testosterone decreased:0.39), (TROSPIUM CHLORIDE:Blood testosterone decreased:0.04), (UMECLIDINIUM BROMIDE\VILANTEROL TRIFENATATE:Blood testosterone decreased:1.43), (ACARBOSE:Blood triglycerides:0.26), (AMLODIPINE BESYLATE\HYDROCHLOROTHIAZIDE\OLMESARTAN MEDOXOMIL:Blood triglycerides:0.15), (BISACODYL:Blood triglycerides:0.19), (BISACODYL OR DOCUSATE SODIUM:Blood triglycerides:0.57), (BUDESONIDE\FORMOTEROL FUMARATE:Blood triglycerides:0.18), (CALCIUM ACETATE:Blood triglycerides:0.25), (CANAGLIFLOZIN\METFORMIN HYDROCHLORIDE:Blood triglycerides:0.16), (CHOLESTYRAMINE:Blood triglycerides:0.66), (CLOTRIMAZOLE:Blood triglycerides:0.33), (CORTICOTROPIN:Blood triglycerides:0), (CORTISONE\HYDROCORTISONE:Blood triglycerides:0.4), (DACLIZUMAB:Blood triglycerides:0.3), (DASATINIB:Blood triglycerides:0.33), (DICYCLOMINE HYDROCHLORIDE:Blood triglycerides:0.77), (DIPHENHYDRAMINE:Blood triglycerides:0.69), (DOXEPIN HYDROCHLORIDE:Blood triglycerides:0), (ELETRIPTAN HYDROBROMIDE:Blood triglycerides:0.66), (EMPAGLIFLOZIN\LINAGLIPTIN:Blood triglycerides:0.81), (EMPAGLIFLOZIN\METFORMIN HYDROCHLORIDE:Blood triglycerides:0.35), (ENOXAPARIN:Blood triglycerides:0.46), (FERROUS SULFATE\FERROUS SULFATE, DRIED:Blood triglycerides:1.35), (FESOTERODINE FUMARATE:Blood triglycerides:0.39), (HYDROCHLOROTHIAZIDE\IRBESARTAN:Blood triglycerides:0.43), (HYDROCHLOROTHIAZIDE\TELMISARTAN:Blood triglycerides:0.04), (HYOSCYAMINE:Blood triglycerides:0.32), (ILOPROST:Blood triglycerides:0.2), (IPRATROPIUM:Blood triglycerides:0.18), (LEDIPASVIR\SOFOSBUVIR:Blood triglycerides:0.11), (LERCANIDIPINE:Blood triglycerides:0.45), (LERCANIDIPINE HYDROCHLORIDE:Blood triglycerides:0.14), (LINACLOTIDE:Blood triglycerides:0.11), (METHENAMINE HIPPURATE:Blood triglycerides:0.23), (METHIMAZOLE:Blood triglycerides:0.11), (METHYLPREDNISOLONE ACETATE:Blood triglycerides:0.01), (METHYLPREDNISOLONE SODIUM SUCCINATE:Blood triglycerides:0.36), (METRONIDAZOLE:Blood triglycerides:0.7), (MIDODRINE:Blood triglycerides:0.02), (NALOXONE HYDROCHLORIDE\OXYCODONE HYDROCHLORIDE:Blood triglycerides:0.11), (NATEGLINIDE:Blood triglycerides:0.07), (NEBIVOLOL:Blood triglycerides:0.52), (NILOTINIB:Blood triglycerides:0.2), (OXYBUTYNIN CHLORIDE:Blood triglycerides:0.33), (PAROXETINE:Blood triglycerides:0.4), (PAROXETINE\PAROXETINE HYDROCHLORIDE:Blood triglycerides:0.4), (PEGINTERFERON BETA-1A:Blood triglycerides:0.09), (PENICILLIN:Blood triglycerides:0.01), (PERINDOPRIL:Blood triglycerides:0.08), (PRAMIPEXOLE DIHYDROCHLORIDE:Blood triglycerides:0.77), (PROMETHAZINE\PROMETHAZINE HYDROCHLORIDE:Blood triglycerides:0.64), (QUETIAPINE:Blood triglycerides:0.15), (RABEPRAZOLE:Blood triglycerides:0.28), (RALOXIFENE HYDROCHLORIDE:Blood triglycerides:0.66), (RIZATRIPTAN BENZOATE:Blood triglycerides:0.2), (SUMATRIPTAN:Blood triglycerides:0.2), (SUMATRIPTAN\SUMATRIPTAN SUCCINATE:Blood triglycerides:0.39), (TETRACYCLINE:Blood triglycerides:0.29), (THALIDOMIDE:Blood triglycerides:0.03), (THYROID, PORCINE:Blood triglycerides:0.2), (TIZANIDINE HYDROCHLORIDE:Blood triglycerides:0.76), (TOLTERODINE TARTRATE:Blood triglycerides:0.32), (ZOPICLONE:Blood triglycerides:0.1), (ACARBOSE:Blood triglycerides abnormal:0.18), (ACETAMINOPHEN\BUTALBITAL\CAFFEINE:Blood triglycerides abnormal:0.29), (ATROPINE SULFATE\DIPHENOXYLATE HYDROCHLORIDE:Blood triglycerides abnormal:0.33), (BUPRENORPHINE HYDROCHLORIDE:Blood triglycerides abnormal:0.23), (CALCIUM ACETATE:Blood triglycerides abnormal:0.13), (CANAGLIFLOZIN\METFORMIN HYDROCHLORIDE:Blood triglycerides abnormal:0.03), (CANDESARTAN:Blood triglycerides abnormal:0.94), (CARISOPRODOL:Blood triglycerides abnormal:0.02), (CARVEDILOL PHOSPHATE:Blood triglycerides abnormal:0.27), (CHOLESTYRAMINE:Blood triglycerides abnormal:0.5), (CLOTRIMAZOLE:Blood triglycerides abnormal:0.21), (COLCHICINE:Blood triglycerides abnormal:0.53), (DALFAMPRIDINE:Blood triglycerides abnormal:0.23), (DAPAGLIFLOZIN PROPANEDIOL:Blood triglycerides abnormal:0.66), (DESLORATADINE:Blood triglycerides abnormal:0.05), (DUTASTERIDE:Blood triglycerides abnormal:0.68), (EDOXABAN TOSYLATE:Blood triglycerides abnormal:0.05), (ELETRIPTAN HYDROBROMIDE:Blood triglycerides abnormal:0.26), (EMPAGLIFLOZIN\LINAGLIPTIN:Blood triglycerides abnormal:0.45), (EMPAGLIFLOZIN\METFORMIN HYDROCHLORIDE:Blood triglycerides abnormal:0.14), (ENOXAPARIN:Blood triglycerides abnormal:0.1), (ESZOPICLONE:Blood triglycerides abnormal:0.53), (FESOTERODINE FUMARATE:Blood triglycerides abnormal:0.22), (FLUTICASONE:Blood triglycerides abnormal:0.11), (FLUTICASONE\FLUTICASONE PROPIONATE:Blood triglycerides abnormal:0.06), (GLUCOSAMINE:Blood triglycerides abnormal:0.03), (HYDROMORPHONE HYDROCHLORIDE:Blood triglycerides abnormal:0.14), (HYDROXYZINE PAMOATE:Blood triglycerides abnormal:0.53), (HYOSCYAMINE:Blood triglycerides abnormal:0.16), (IBRUTINIB:Blood triglycerides abnormal:0.69), (ILOPROST:Blood triglycerides abnormal:0.06), (INDOMETHACIN:Blood triglycerides abnormal:0.29), (INSULIN DEGLUDEC:Blood triglycerides abnormal:0.16), (LEDIPASVIR\SOFOSBUVIR:Blood triglycerides abnormal:0.35), (LENALIDOMIDE:Blood triglycerides abnormal:0.2), (LIDOCAINE:Blood triglycerides abnormal:0.15), (LOMITAPIDE MESYLATE:Blood triglycerides abnormal:0.06), (METHADONE HYDROCHLORIDE:Blood triglycerides abnormal:0.37), (METHYLPHENIDATE HYDROCHLORIDE:Blood triglycerides abnormal:0.4), (METHYLPREDNISOLONE ACETATE:Blood triglycerides abnormal:0.04), (METHYLPREDNISOLONE SODIUM SUCCINATE:Blood triglycerides abnormal:0.18), (NADOLOL:Blood triglycerides abnormal:0.16), (NALOXONE HYDROCHLORIDE\OXYCODONE HYDROCHLORIDE:Blood triglycerides abnormal:0.15), (NAPROXEN SODIUM:Blood triglycerides abnormal:0.51), (OXYBUTYNIN CHLORIDE:Blood triglycerides abnormal:0), (PENICILLIN:Blood triglycerides abnormal:0.04), (POLYETHYLENE GLYCOLS:Blood triglycerides abnormal:0.52), (RABEPRAZOLE:Blood triglycerides abnormal:0.17), (RALOXIFENE HYDROCHLORIDE:Blood triglycerides abnormal:0.65), (RANOLAZINE:Blood triglycerides abnormal:1.79), (ROPINIROLE HYDROCHLORIDE:Blood triglycerides abnormal:0.13), (SCOPOLAMINE:Blood triglycerides abnormal:0.19), (SECUKINUMAB:Blood triglycerides abnormal:0.23), (SUMATRIPTAN\SUMATRIPTAN SUCCINATE:Blood triglycerides abnormal:0.1), (TERIFLUNOMIDE:Blood triglycerides abnormal:0.46), (TETRACYCLINE:Blood triglycerides abnormal:0.22), (TOCILIZUMAB:Blood triglycerides abnormal:0.39), (TRIAMTERENE:Blood triglycerides abnormal:0.12), (UMECLIDINIUM BROMIDE\VILANTEROL TRIFENATATE:Blood triglycerides abnormal:0.56), (VILAZODONE HYDROCHLORIDE:Blood triglycerides abnormal:0.08), (ACETAMINOPHEN\OXYCODONE\OXYCODONE HYDROCHLORIDE:Blood triglycerides increased:0.67), (ALCOHOL:Blood triglycerides increased:0.1), (ALFACALCIDOL:Blood triglycerides increased:0.16), (AMANTADINE:Blood triglycerides increased:0.41), (BECLOMETHASONE DIPROPIONATE:Blood triglycerides increased:0.17), (BICALUTAMIDE:Blood triglycerides increased:0.21), (BUDESONIDE\FORMOTEROL FUMARATE:Blood triglycerides increased:0.44), (BUPRENORPHINE HYDROCHLORIDE\NALOXONE HYDROCHLORIDE:Blood triglycerides increased:0.8), (CALCIUM\MAGNESIUM\ZINC:Blood triglycerides increased:0.89), (CANDESARTAN:Blood triglycerides increased:1.42), (CARVEDILOL PHOSPHATE:Blood triglycerides increased:1.59), (CLOTRIMAZOLE:Blood triglycerides increased:0.84), (CODEINE:Blood triglycerides increased:0.24), (CORTICOTROPIN:Blood triglycerides increased:0.55), (DABIGATRAN:Blood triglycerides increased:0.29), (DACLIZUMAB:Blood triglycerides increased:0.63), (DARBEPOETIN ALFA:Blood triglycerides increased:0.17), (DASATINIB:Blood triglycerides increased:0.94), (DEFLAZACORT:Blood triglycerides increased:0.25), (DESONIDE:Blood triglycerides increased:0.28), (DEXTROAMPHETAMINE SULFATE:Blood triglycerides increased:0.31), (DRONABINOL:Blood triglycerides increased:0.52), (EDOXABAN TOSYLATE:Blood triglycerides increased:0.61), (EMPAGLIFLOZIN\METFORMIN HYDROCHLORIDE:Blood triglycerides increased:0.39), (EPINEPHRINE:Blood triglycerides increased:1.2), (ESOMEPRAZOLE MAGNESIUM\NAPROXEN:Blood triglycerides increased:0.55), (ESTROGENS:Blood triglycerides increased:0.25), (FORMOTEROL FUMARATE:Blood triglycerides increased:0.06), (FUSIDIC ACID:Blood triglycerides increased:0.04), (GLYCOPYRRONIUM\INDACATEROL:Blood triglycerides increased:0.21), (GUANFACINE:Blood triglycerides increased:0.09), (HYDROCODONE BITARTRATE\IBUPROFEN:Blood triglycerides increased:0.32), (HYDROXYCHLOROQUINE DIPHOSPHATE:Blood triglycerides increased:0.15), (ILOPROST:Blood triglycerides increased:0.17), (IMATINIB MESYLATE:Blood triglycerides increased:0.14), (IMIPRAMINE HYDROCHLORIDE:Blood triglycerides increased:0.77), (INDAPAMIDE\PERINDOPRIL:Blood triglycerides increased:0.1), (INDOMETHACIN:Blood triglycerides increased:0.64), (INFLUENZA VIRUS VACCINE:Blood triglycerides increased:0.04), (LABETALOL\LABETALOL HYDROCHLORIDE:Blood triglycerides increased:1.12), (LERCANIDIPINE:Blood triglycerides increased:0.84), (LERCANIDIPINE HYDROCHLORIDE:Blood triglycerides increased:0.39), (LEVOMEFOLATE CALCIUM:Blood triglycerides increased:0.43), (LIDOCAINE HYDROCHLORIDE:Blood triglycerides increased:0.7), (MEDROXYPROGESTERONE ACETATE:Blood triglycerides increased:0.66), (METHENAMINE HIPPURATE:Blood triglycerides increased:0.47), (METHIMAZOLE:Blood triglycerides increased:0.23), (METHYLPREDNISOLONE ACETATE:Blood triglycerides increased:1.13), (NATEGLINIDE:Blood triglycerides increased:0.64), (NICOTINE:Blood triglycerides increased:0.27), (NILOTINIB:Blood triglycerides increased:0.49), (NORETHINDRONE:Blood triglycerides increased:0.13), (NORTRIPTYLINE HYDROCHLORIDE:Blood triglycerides increased:0.27), (OLMESARTAN:Blood triglycerides increased:0.04), (OXYBUTYNIN\OXYBUTYNIN CHLORIDE:Blood triglycerides increased:0.25), (PANTOPRAZOLE MAGNESIUM:Blood triglycerides increased:0.97), (PATIROMER:Blood triglycerides increased:0.43), (PAZOPANIB HYDROCHLORIDE:Blood triglycerides increased:0.38), (PEGINTERFERON BETA-1A:Blood triglycerides increased:0.83), (PIMECROLIMUS:Blood triglycerides increased:0.21), (PRAZOSIN HYDROCHLORIDE:Blood triglycerides increased:0.29), (PREDNISOLONE ACETATE:Blood triglycerides increased:0.57), (PROPAFENONE HYDROCHLORIDE:Blood triglycerides increased:0.41), (PROTRIPTYLINE HYDROCHLORIDE:Blood triglycerides increased:0.03), (RABEPRAZOLE:Blood triglycerides increased:0.62), (SACCHAROMYCES CEREVISIAE:Blood triglycerides increased:0.1), (SALMETEROL XINAFOATE:Blood triglycerides increased:0.14), (SCOPOLAMINE:Blood triglycerides increased:0.26), (SECUKINUMAB:Blood triglycerides increased:0.81), (SENNOSIDES:Blood triglycerides increased:0.21), (SITAGLIPTIN:Blood triglycerides increased:0.02), (SODIUM PHOSPHATE, DIBASIC\SODIUM PHOSPHATE, MONOBASIC:Blood triglycerides increased:0.01), (SUMATRIPTAN:Blood triglycerides increased:0.89), (TERBUTALINE SULFATE:Blood triglycerides increased:0.44), (TIMOLOL MALEATE:Blood triglycerides increased:0.02), (TOLTERODINE:Blood triglycerides increased:0.11), (VALGANCICLOVIR HYDROCHLORIDE:Blood triglycerides increased:0.16), (ZOPICLONE:Blood triglycerides increased:0.66), (ALBUTEROL SULFATE:Blood uric acid:0.01), (BUDESONIDE:Blood uric acid:0.18), (CALCIUM CARBONATE\CHOLECALCIFEROL:Blood uric acid:0.42), (CANDESARTAN:Blood uric acid:0.46), (CANDESARTAN CILEXETIL\HYDROCHLOROTHIAZIDE:Blood uric acid:0.04), (DEXLANSOPRAZOLE:Blood uric acid:0.17), (DICLOFENAC:Blood uric acid:0.25), (EVOLOCUMAB:Blood uric acid:0.69), (FERROUS SULFATE\FERROUS SULFATE, DRIED:Blood uric acid:0.55), (MELOXICAM:Blood uric acid:0.19), (PRAVASTATIN:Blood uric acid:0.33), (RANOLAZINE:Blood uric acid:0.49), (SACUBITRIL\VALSARTAN:Blood uric acid:0.63), (SERTRALINE HYDROCHLORIDE:Blood uric acid:0.05), (TERIFLUNOMIDE:Blood uric acid:0.03), (TRAMADOL:Blood uric acid:0.49), (TRAZODONE HYDROCHLORIDE:Blood uric acid:0.03), (ANASTROZOLE:Blood uric acid abnormal:0.09), (BUDESONIDE:Blood uric acid abnormal:0.04), (DASATINIB:Blood uric acid abnormal:0.03), (DEVICE:Blood uric acid abnormal:0.08), (DEXLANSOPRAZOLE:Blood uric acid abnormal:0.06), (DICLOFENAC SODIUM:Blood uric acid abnormal:0.37), (DILTIAZEM:Blood uric acid abnormal:0.08), (ERGOCALCIFEROL:Blood uric acid abnormal:0.03), (EVOLOCUMAB:Blood uric acid abnormal:0.64), (FERROUS SULFATE\FERROUS SULFATE, DRIED:Blood uric acid abnormal:0.35), (GLIPIZIDE:Blood uric acid abnormal:0.3), (IMATINIB MESYLATE:Blood uric acid abnormal:0.26), (LEDIPASVIR\SOFOSBUVIR:Blood uric acid abnormal:0.01), (LERCANIDIPINE:Blood uric acid abnormal:0.29), (LOVASTATIN:Blood uric acid abnormal:0.17), (MELOXICAM:Blood uric acid abnormal:0.04), (PRAVASTATIN:Blood uric acid abnormal:0.36), (PRAVASTATIN SODIUM:Blood uric acid abnormal:0.44), (TERIFLUNOMIDE:Blood uric acid abnormal:0.01), (TRAMADOL:Blood uric acid abnormal:0.4), (VENLAFAXINE HYDROCHLORIDE:Blood uric acid abnormal:0.03), (ALENDRONIC ACID:Blood uric acid increased:0.21), (AMPHETAMINE ASPARTATE\AMPHETAMINE SULFATE\DEXTROAMPHETAMINE SACCHARATE\DEXTROAMPHETAMINE SULFATE:Blood uric acid increased:0.19), (ANASTROZOLE:Blood uric acid increased:0.88), (ATOVAQUONE:Blood uric acid increased:0.08), (BENAZEPRIL HYDROCHLORIDE:Blood uric acid increased:0.6), (BICYCLOL:Blood uric acid increased:0.23), (BISACODYL OR DOCUSATE SODIUM:Blood uric acid increased:0.61), (BUDESONIDE\FORMOTEROL FUMARATE:Blood uric acid increased:0.13), (BUPROPION:Blood uric acid increased:0.2), (BUPROPION HYDROCHLORIDE:Blood uric acid increased:1.1), (BUSPIRONE HYDROCHLORIDE:Blood uric acid increased:0.05), (CALCIUM ACETATE:Blood uric acid increased:0.82), (CANAGLIFLOZIN\METFORMIN HYDROCHLORIDE:Blood uric acid increased:0.07), (CARVEDILOL PHOSPHATE:Blood uric acid increased:0.1), (CEFOTAXIME SODIUM:Blood uric acid increased:0.01), (CEPHALEXIN:Blood uric acid increased:1.35), (CHOLESTYRAMINE:Blood uric acid increased:0.43), (CILOSTAZOL:Blood uric acid increased:0.04), (CLOTRIMAZOLE:Blood uric acid increased:0.42), (CLOZAPINE:Blood uric acid increased:0.25), (CORTISONE\HYDROCORTISONE:Blood uric acid increased:0.99), (DANAZOL:Blood uric acid increased:0.3), (DIVALPROEX SODIUM:Blood uric acid increased:0.28), (DOFETILIDE:Blood uric acid increased:0.35), (EDOXABAN TOSYLATE:Blood uric acid increased:0.16), (EPLERENONE:Blood uric acid increased:0.44), (ETIMICIN SULFATE:Blood uric acid increased:0.3), (EVOLOCUMAB:Blood uric acid increased:1.31), (FLUTICASONE FUROATE\SALMETEROL XINAFOATE:Blood uric acid increased:0.16), (HYDROXYCHLOROQUINE DIPHOSPHATE:Blood uric acid increased:0.02), (HYDROXYCHLOROQUINE SULFATE:Blood uric acid increased:0.71), (HYDROXYZINE PAMOATE:Blood uric acid increased:0.09), (INDAPAMIDE\PERINDOPRIL:Blood uric acid increased:0.83), (INSULIN DEGLUDEC:Blood uric acid increased:0.16), (ITRACONAZOLE:Blood uric acid increased:0.07), (LEDIPASVIR\SOFOSBUVIR:Blood uric acid increased:0.79), (LERCANIDIPINE HYDROCHLORIDE:Blood uric acid increased:0.66), (LEUCOGEN:Blood uric acid increased:0.24), (LEVALBUTEROL HYDROCHLORIDE:Blood uric acid increased:0.26), (METHENAMINE HIPPURATE:Blood uric acid increased:0.06), (METHOCARBAMOL:Blood uric acid increased:0.05), (MIDODRINE:Blood uric acid increased:0.16), (NATEGLINIDE:Blood uric acid increased:0.15), (NORTRIPTYLINE:Blood uric acid increased:0.1), (OLANZAPINE:Blood uric acid increased:0.17), (OXCARBAZEPINE:Blood uric acid increased:0.36), (OXYBUTYNIN CHLORIDE:Blood uric acid increased:0.12), (OXYGEN:Blood uric acid increased:1.22), (PANTOPRAZOLE MAGNESIUM:Blood uric acid increased:0.55), (PRAVASTATIN SODIUM:Blood uric acid increased:1.15), (PRIMIDONE:Blood uric acid increased:0.14), (SUCRALFATE:Blood uric acid increased:0.26), (TIZANIDINE HYDROCHLORIDE:Blood uric acid increased:0.29), (TRIAMCINOLONE ACETONIDE:Blood uric acid increased:0.16), (UMECLIDINIUM BROMIDE\VILANTEROL TRIFENATATE:Blood uric acid increased:0.39), (VANCOMYCIN HYDROCHLORIDE:Blood uric acid increased:0.39), (VARENICLINE TARTRATE:Blood uric acid increased:0.17), (AMIODARONE:Body temperature increased:0.2), (BUMETANIDE:Body temperature increased:0.51), (CALCITRIOL:Body temperature increased:0.05), (CELECOXIB:Body temperature increased:0), (FISH OIL:Body temperature increased:0.22), (ISOSORBIDE MONONITRATE:Body temperature increased:0.12), (LAMOTRIGINE:Body temperature increased:0.28), (LOVASTATIN:Body temperature increased:0.17), (NAPROXEN:Body temperature increased:0.32), (PANTOPRAZOLE:Body temperature increased:0.3), (SACUBITRIL\VALSARTAN:Body temperature increased:0.21), (TAMSULOSIN:Body temperature increased:0.45), (TOPIRAMATE:Body temperature increased:0.72), (VERAPAMIL HYDROCHLORIDE:Body temperature increased:0.27), (ZONISAMIDE:Body temperature increased:0.11), (ACETAMINOPHEN\BUTALBITAL\CAFFEINE:Bone cancer:0.39), (ACETAMINOPHEN\OXYCODONE\OXYCODONE HYDROCHLORIDE:Bone cancer:0.05), (ALENDRONIC ACID:Bone cancer:0.11), (AMITRIPTYLINE:Bone cancer:0.49),(ARMODAFINIL:Bone cancer:0.37), (BENAZEPRIL\BENAZEPRIL HYDROCHLORIDE:Bone cancer:0.16), (BISOPROLOL FUMARATE:Bone cancer:0.09), (BUDESONIDE:Bone cancer:1.03), (BUMETANIDE:Bone cancer:0.75), (CALCITONIN SALMON:Bone cancer:0.07), (CALCITRIOL:Bone cancer:0.36), (CANDESARTAN:Bone cancer:0.62), (CANDESARTAN CILEXETIL\HYDROCHLOROTHIAZIDE:Bone cancer:0.15), (CARVEDILOL PHOSPHATE:Bone cancer:0.53), (CERTOLIZUMAB PEGOL:Bone cancer:0.54), (CLOZAPINE:Bone cancer:0.02), (COLCHICINE:Bone cancer:0.37), (CORTISONE\HYDROCORTISONE:Bone cancer:0.41), (DALFAMPRIDINE:Bone cancer:0.21), (DARATUMUMAB:Bone cancer:0.03), (DEXLANSOPRAZOLE:Bone cancer:1.01), (ELETRIPTAN HYDROBROMIDE:Bone cancer:0.15), (EPINEPHRINE:Bone cancer:0.67), (EVOLOCUMAB:Bone cancer:0.61), (FERROUS SULFATE\FERROUS SULFATE, DRIED:Bone cancer:0.98), (GIMERACIL\OTERACIL\TEGAFUR:Bone cancer:0.37), (HERBALS\TURMERIC:Bone cancer:0.28), (HYOSCYAMINE:Bone cancer:0.06), (IBRUTINIB:Bone cancer:1.32), (IDELALISIB:Bone cancer:0.26), (INDOMETHACIN:Bone cancer:0.13), (ISOSORBIDE:Bone cancer:0.35), (LEUCOVORIN CALCIUM:Bone cancer:0.78), (LISDEXAMFETAMINE DIMESYLATE:Bone cancer:0.12), (METAXALONE:Bone cancer:0.43), (NIRAPARIB:Bone cancer:0.46), (OLAPARIB:Bone cancer:0.96), (OMEGA-3-ACID ETHYL ESTERS:Bone cancer:0.38), (OXYBUTYNIN CHLORIDE:Bone cancer:0.32), (PANITUMUMAB:Bone cancer:0.33), (PANTOPRAZOLE MAGNESIUM:Bone cancer:0.25), (PENICILLIN:Bone cancer:0.29), (PHENOBARBITAL:Bone cancer:0.25), (PONATINIB:Bone cancer:0.33), (PRAMIPEXOLE DIHYDROCHLORIDE:Bone cancer:0.03), (PRIMIDONE:Bone cancer:0.58), (PROGESTERONE:Bone cancer:0.13), (QUETIAPINE FUMARATE:Bone cancer:0.09), (RABEPRAZOLE:Bone cancer:0.02), (RADIATION THERAPY:Bone cancer:0.95), (RALOXIFENE HYDROCHLORIDE:Bone cancer:0.29), (RAMUCIRUMAB:Bone cancer:0.08), (RANOLAZINE:Bone cancer:0.58), (RISEDRONATE SODIUM:Bone cancer:0.5), (ROFLUMILAST:Bone cancer:0.09), (SACUBITRIL\VALSARTAN:Bone cancer:0.33), (SODIUM OXYBATE:Bone cancer:0.48), (SOTALOL:Bone cancer:0.13), (TELMISARTAN:Bone cancer:0.44), (TERIFLUNOMIDE:Bone cancer:0.28), (TINZAPARIN SODIUM:Bone cancer:0.14), (TORSEMIDE:Bone cancer:0.52), (TRIAMCINOLONE ACETONIDE:Bone cancer:0.72), (UMECLIDINIUM BROMIDE\VILANTEROL TRIFENATATE:Bone cancer:0.36), (VINCRISTINE SULFATE:Bone cancer:0.03), (ALBUTEROL SULFATE\IPRATROPIUM BROMIDE:Bone density abnormal:0.12), (CALCIUM CARBONATE\VITAMIN D:Bone density abnormal:0.02), (CANDESARTAN CILEXETIL:Bone density abnormal:0.01), (CLINDAMYCIN\CLINDAMYCIN PHOSPHATE:Bone density abnormal:0.45), (COLCHICINE:Bone density abnormal:0.3), (DACLIZUMAB:Bone density abnormal:0.03), (DICLOFENAC:Bone density abnormal:0.45), (ELETRIPTAN HYDROBROMIDE:Bone density abnormal:0.49), (ESOMEPRAZOLE:Bone density abnormal:0), (EVOLOCUMAB:Bone density abnormal:0.59), (HYOSCYAMINE:Bone density abnormal:0.15), (INSULIN HUMAN:Bone density abnormal:0.27), (LISDEXAMFETAMINE DIMESYLATE:Bone density abnormal:0.35), (METAXALONE:Bone density abnormal:0.57), (METRONIDAZOLE\METRONIDAZOLE HYDROCHLORIDE:Bone density abnormal:0.23), (OLANZAPINE:Bone density abnormal:0.14), (OXCARBAZEPINE:Bone density abnormal:0.02), (PANTOPRAZOLE MAGNESIUM:Bone density abnormal:0.22), (POLYETHYLENE GLYCOLS:Bone density abnormal:0.21), (PRIMIDONE:Bone density abnormal:0.05), (RANOLAZINE:Bone density abnormal:0.63), (SACUBITRIL\VALSARTAN:Bone density abnormal:0.51), (SODIUM CHLORIDE:Bone density abnormal:0.02), (SUMATRIPTAN:Bone density abnormal:0.01), (TOCILIZUMAB:Bone density abnormal:0.45), (TORSEMIDE:Bone density abnormal:0.01), (TRIAMTERENE:Bone density abnormal:0.23), (VARENICLINE TARTRATE:Bone density abnormal:0.81), (AMPHETAMINE ASPARTATE\AMPHETAMINE SULFATE\DEXTROAMPHETAMINE SACCHARATE\DEXTROAMPHETAMINE SULFATE:Bone density decreased:0.05), (BORTEZOMIB:Bone density decreased:0.08), (DALFAMPRIDINE:Bone density decreased:0.82), (DASATINIB:Bone density decreased:0.03), (DEXLANSOPRAZOLE:Bone density decreased:0.85), (DIPHENHYDRAMINE:Bone density decreased:0.04), (ESZOPICLONE:Bone density decreased:0.28), (EVOLOCUMAB:Bone density decreased:0.48), (FERROUS SULFATE:Bone density decreased:0.15), (FERROUS SULFATE\FERROUS SULFATE, DRIED:Bone density decreased:0.31), (FINGOLIMOD HYDROCHLORIDE:Bone density decreased:0.04), (HEPARIN SODIUM:Bone density decreased:0.01), (IBRUTINIB:Bone density decreased:0.37), (INDOMETHACIN:Bone density decreased:0.04), (METAXALONE:Bone density decreased:0.22), (PRAVASTATIN:Bone density decreased:0.34), (PROMETHAZINE\PROMETHAZINE HYDROCHLORIDE:Bone density decreased:0.1), (PYRIDOXINE:Bone density decreased:0.15), (RANOLAZINE:Bone density decreased:0.15), (SACUBITRIL\VALSARTAN:Bone density decreased:0.19), (SECUKINUMAB:Bone density decreased:0.13), (SODIUM OXYBATE:Bone density decreased:0.35), (SPIRONOLACTONE:Bone density decreased:0.9), (TRIAMTERENE:Bone density decreased:0.08), (.ALPHA.1-PROTEINASE INHIBITOR HUMAN:Bone disorder:0.59), (ACETAMINOPHEN\METHOCARBAMOL:Bone disorder:0.14), (ADO-TRASTUZUMAB EMTANSINE:Bone disorder:1.42), (ALBIGLUTIDE:Bone disorder:0.23), (ALCOHOL:Bone disorder:0.6), (ALISKIREN:Bone disorder:0.26), (ALISKIREN HEMIFUMARATE\HYDROCHLOROTHIAZIDE:Bone disorder:0.03), (ALUMINUM HYDROXIDE\MAGNESIUM HYDROXIDE\SIMETHICONE:Bone disorder:0.03), (AMMONIUM CHLORIDE\DEXTROMETHORPHAN\SODIUM CITRATE:Bone disorder:0.12), (AMPHOTERICIN B:Bone disorder:0.22), (AMPICILLIN:Bone disorder:0.64), (ANAKINRA:Bone disorder:0.31), (ANHYDROUS CITRIC ACID\ASPIRIN\SODIUM BICARBONATE:Bone disorder:0.04), (ANTIHISTAMINES NOS:Bone disorder:0.44), (ASPIRIN LYSINE:Bone disorder:0.03), (ASPIRIN\BUTALBITAL\CAFFEINE:Bone disorder:0.13), (ATROPINE SULFATE\HYOSCYAMINE SULFATE\PHENOBARBITAL\SCOPOLAMINE HYDROBROMIDE:Bone disorder:0.03), (BACITRACIN:Bone disorder:1.05), (BAMIFYLLINE HYDROCHLORIDE:Bone disorder:0.34), (BENAZEPRIL\BENAZEPRIL HYDROCHLORIDE:Bone disorder:1.77), (BETAMETHASONE DIPROPIONATE\CALCIPOTRIENE\CALCIPOTRIENE HYDRATE:Bone disorder:0.87), (BUPRENORPHINE HYDROCHLORIDE:Bone disorder:0.75), (CABOZANTINIB S-MALATE:Bone disorder:0.08), (CALCIUM CARBONATE\ERGOCALCIFEROL\RETINOL:Bone disorder:0.07), (CARBIDOPA:Bone disorder:0.18), (CEFAZOLIN:Bone disorder:0.44), (CEFOTAXIME SODIUM:Bone disorder:0.14), (CEFUROXIME:Bone disorder:0.41), (CERITINIB:Bone disorder:0.02), (CHLORAMBUCIL:Bone disorder:0.07), (CLINDAMYCIN HYDROCHLORIDE:Bone disorder:0.29), (CORTICOTROPIN:Bone disorder:0.99), (CYANOCOBALAMIN\FOLIC ACID\PYRIDOXINE:Bone disorder:0.13), (DACLIZUMAB:Bone disorder:0.89), (DANAZOL:Bone disorder:0.67), (DAPAGLIFLOZIN PROPANEDIOL\METFORMIN HYDROCHLORIDE:Bone disorder:0.07), (DASATINIB:Bone disorder:2.46), (DEXAMETHASONE ACETATE:Bone disorder:0.14), (DEXPANTHENOL\NIACINAMIDE\PYRIDOXINE HYDROCHLORIDE\RIBOFLAVIN 5′-PHOSPHATE SODIUM\THIAMINE HYDROCHLORIDE:Bone disorder:0.2), (DIGITOXIN:Bone disorder:0.28), (DIPHENHYDRAMINE HYDROCHLORIDE\LIDOCAINE HYDROCHLORIDE\NYSTATIN:Bone disorder:0.07), (DOLASETRON MESYLATE:Bone disorder:0.68), (DUTASTERIDE\TAMSULOSIN HYDROCHLORIDE:Bone disorder:0.63), (ECONAZOLE:Bone disorder:0.38), (EDOXABAN TOSYLATE:Bone disorder:1.07), (EMPAGLIFLOZIN\LINAGLIPTIN:Bone disorder:1.15), (EPLERENONE:Bone disorder:1.09), (ERIBULIN:Bone disorder:0.09), (ETHINYL ESTRADIOL\NORGESTREL:Bone disorder:0.2), (FENOTEROL HYDROBROMIDE\IPRATROPIUM BROMIDE:Bone disorder:0.11), (FERRIC POLYSACCHARIDE COMPLEX:Bone disorder:0.08), (FERROUS SULFATE\GLYCINE:Bone disorder:0.02), (FISH OIL\TOCOPHEROL:Bone disorder:0.04), (FLUOCINOLONE ACETONIDE:Bone disorder:0.61), (FLUOROMETHOLONE:Bone disorder:0.09), (FORMOTEROL FUMARATE\MOMETASONE FUROATE:Bone disorder:1.25), (FOSINOPRIL:Bone disorder:0.13), (FOSINOPRIL SODIUM:Bone disorder:0.21), (GEMCITABINE HYDROCHLORIDE:Bone disorder:0.36), (GIMERACIL\OTERACIL\TEGAFUR:Bone disorder:0.02), (GLIMEPIRIDE\ROSIGLITAZONE MALEATE:Bone disorder:0.02), (GLIPIZIDE\METFORMIN HYDROCHLORIDE:Bone disorder:0.29), (GLYCOPYRROLATE\INDACATEROL MALEATE:Bone disorder:0.39), (GOLD:Bone disorder:0.06), (IDELALISIB:Bone disorder:0.38), (ILOPROST:Bone disorder:0.57), (INDAPAMIDE\PERINDOPRIL:Bone disorder:1.21), (INFLIXIMAB-DYYB:Bone disorder:0.19), (INSULIN PORK\INSULIN PURIFIED PORK:Bone disorder:0.18), (INTERFERON:Bone disorder:0.37), (INTERFERON ALFA-2A:Bone disorder:0.6), (INTERFERON ALFA-2B:Bone disorder:0.08), (INTERFERON GAMMA-1B:Bone disorder:0.12), (INVESTIGATIONAL PRODUCT:Bone disorder:0.03), (IRINOTECAN:Bone disorder:0.38), (IRON\POLYSACCHARIDES:Bone disorder:0.33), (LACTOBACILLUS ACIDOPHILUS\LACTOBACILLUS DELBRUECKII SUBSP. BULGARICUS:Bone disorder:0.13), (LAPATINIB DITOSYLATE:Bone disorder:0.64), (LEDIPASVIR\SOFOSBUVIR:Bone disorder:1.18), (LIDOCAINE HYDROCHLORIDE:Bone disorder:1.92), (LISDEXAMFETAMINE DIMESYLATE:Bone disorder:0.76), (LOMITAPIDE MESYLATE:Bone disorder:1.04), (LORNOXICAM:Bone disorder:0.04), (LURASIDONE HYDROCHLORIDE:Bone disorder:0.1), (LUTETIUM LU-177:Bone disorder:0.17), (MEPERIDINE:Bone disorder:0.21), (MEROPENEM:Bone disorder:0.18), (METFORMIN HYDROCHLORIDE\ROSIGLITAZONE MALEATE:Bone disorder:0.36), (METFORMIN HYDROCHLORIDE\SAXAGLIPTIN HYDROCHLORIDE:Bone disorder:0.34), (METHENAMINE HIPPURATE:Bone disorder:1.81), (MICONAZOLE NITRATE:Bone disorder:0.33), (MIDODRINE HYDROCHLORIDE:Bone disorder:0.41), (MINOCYCLINE HYDROCHLORIDE:Bone disorder:0.49), (MOEXIPRIL HYDROCHLORIDE:Bone disorder:0.23), (MOXONIDINE:Bone disorder:0.23), (NALOXONE HYDROCHLORIDE\TILIDINE HYDROCHLORIDE:Bone disorder:0.75), (NALTREXONE:Bone disorder:0.29), (NARATRIPTAN HYDROCHLORIDE:Bone disorder:0.27), (NIRAPARIB:Bone disorder:0.5), (NISOLDIPINE:Bone disorder:0.21), (OLODATEROL HYDROCHLORIDE\TIOTROPIUM BROMIDE MONOHYDRATE:Bone disorder:1.52), (OPIPRAMOL:Bone disorder:0.2), (OXYBUTYNIN\OXYBUTYNIN CHLORIDE:Bone disorder:0.76), (OXYMETAZOLINE:Bone disorder:0.61), (PAPAYA:Bone disorder:0.04), (PARATHYROID HORMONE:Bone disorder:0.29), (PASIREOTIDE:Bone disorder:0.22), (PEGINTERFERON BETA-1A:Bone disorder:1.25), (PEMETREXED:Bone disorder:0.3), (PENICILLIN G:Bone disorder:0.45), (PENTOXIFYLLINE:Bone disorder:0.72), (PHENAZOPYRIDINE HYDROCHLORIDE:Bone disorder:0.29), (PHENPROCOUMON:Bone disorder:0.24), (PHENYTOIN SODIUM:Bone disorder:0.45), (PIMECROLIMUS:Bone disorder:0.52), (PIRFENIDONE:Bone disorder:0.1), (POLYETHYLENE GLYCOL 3350\POTASSIUM CHLORIDE\SODIUM BICARBONATE\SODIUM CHLORIDE\SODIUM SULFATE ANHYDROUS:Bone disorder:0.14), (PRAZOSIN:Bone disorder:0.75), (PRAZOSIN HYDROCHLORIDE:Bone disorder:0.84), (PREDNISOLONE\PREDNISOLONE ACETATE\PREDNISOLONE SODIUM SUCCINATE:Bone disorder:0.07), (PROTRIPTYLINE HYDROCHLORIDE:Bone disorder:0.82), (QUINACRINE:Bone disorder:0.25), (SALMETEROL:Bone disorder:0.28), (SITAGLIPTIN:Bone disorder:0.51), (TAMOXIFEN CITRATE:Bone disorder:1.21), (TERBINAFINE HYDROCHLORIDE:Bone disorder:1.23), (TERBUTALINE SULFATE:Bone disorder:1.43), (TESTOSTERONE UNDECANOATE:Bone disorder:0.57), (THEOPHYLLINE:Bone disorder:0.28), (TIMOLOL MALEATE\TRAVOPROST:Bone disorder:0.13), (TRANDOLAPRIL:Bone disorder:0.79), (TRIMIPRAMINE MALEATE:Bone disorder:0.22), (TROSPIUM CHLORIDE:Bone disorder:0.78), (VALDECOXIB:Bone disorder:0.51), (VARDENAFIL HYDROCHLORIDE TRIHYDRATE:Bone disorder:0.39), (VEMURAFENIB:Bone disorder:0.06), (VINCRISTINE SULFATE:Bone disorder:0.15), (VISMODEGIB:Bone disorder:0.02), (ZALEPLON:Bone disorder:0.31), (ZILEUTON:Bone disorder:0.57), (PANTOPRAZOLE:Bone lesion:0.05), (ACETAMINOPHEN\BUTALBITAL\CAFFEINE:Bone loss:0.29), (AMPHETAMINE ASPARTATE\AMPHETAMINE SULFATE\DEXTROAMPHETAMINE SACCHARATE\DEXTROAMPHETAMINE SULFATE:Bone loss:0.69),(ARMODAFINIL:Bone loss:0.55), (BISACODYL OR DOCUSATE SODIUM:Bone loss:0.12), (CANDESARTAN CILEXETIL\HYDROCHLOROTHIAZIDE:Bone loss:0), (CEPHALEXIN:Bone loss:0.22), (CERTOLIZUMAB PEGOL:Bone loss:0.32), (CIPROFLOXACIN HYDROCHLORIDE:Bone loss:0.19), (CLINDAMYCIN\CLINDAMYCIN PHOSPHATE:Bone loss:0.04), (CLONIDINE:Bone loss:0.46), (COLCHICINE:Bone loss:0.07), (CYCLOBENZAPRINE:Bone loss:0.22), (DACLIZUMAB:Bone loss:0.12), (DICLOFENAC:Bone loss:0.01), (DILTIAZEM HYDROCHLORIDE:Bone loss:0.76), (ELETRIPTAN HYDROBROMIDE:Bone loss:0.44), (ESZOPICLONE:Bone loss:0.34), (EVEROLIMUS:Bone loss:0.52), (EVOLOCUMAB:Bone loss:0.39), (FLUTICASONE:Bone loss:0.08), (FLUTICASONE FUROATE\VILANTEROL TRIFENATATE:Bone loss:0.08), (HYDROCHLOROTHIAZIDE\LISINOPRIL:Bone loss:0.21), (IBRUTINIB:Bone loss:0.99), (ILOPROST:Bone loss:0.15), (INSULIN LISPRO:Bone loss:0.26), (METAXALONE:Bone loss:0.49), (METHYLPREDNISOLONE SODIUM SUCCINATE:Bone loss:0.18), (METRONIDAZOLE:Bone loss:0.07), (NITROGLYCERIN:Bone loss:0.11), (OXCARBAZEPINE:Bone loss:0.11), (PRAMIPEXOLE DIHYDROCHLORIDE:Bone loss:0.18), (PRAVASTATIN SODIUM:Bone loss:0.59), (PRIMIDONE:Bone loss:0.03), (RANOLAZINE:Bone loss:0.54), (SACUBITRIL\VALSARTAN:Bone loss:0.33), (VARENICLINE TARTRATE:Bone loss:0.18), (VITAMIN B:Bone loss:0.4), (ALBUTEROL SULFATE:Bone marrow conditioning regimen:0.34), (ALBUTEROL SULFATE\IPRATROPIUM BROMIDE:Bone marrow conditioning regimen:0), (ALFACALCIDOL:Bone marrow conditioning regimen:0.03), (ANIDULAFUNGIN:Bone marrow conditioning regimen:0.98), (ARSENIC TRIOXIDE:Bone marrow conditioning regimen:0.25), (BELATACEPT:Bone marrow conditioning regimen:0.32), (BLESELUMAB:Bone marrow conditioning regimen:0.01), (BRINCIDOFOVIR:Bone marrow conditioning regimen:1.26), (CALCITRIOL:Bone marrow conditioning regimen:0.22), (CALCIUM CARBONATE\CHOLECALCIFEROL:Bone marrow conditioning regimen:0.1), (CANAKINUMAB:Bone marrow conditioning regimen:0.46), (CEFOZOPRAN:Bone marrow conditioning regimen:0.05), (CYCLOPHOSPHAMIDE\CYTARABINE\DEXAMETHASONE\DOXORUBIC IN\METHOTREXATE\VINCRISTINE:Bone marrow conditioning regimen:0.57), (DACLIZUMAB:Bone marrow conditioning regimen:0.62), (DEXAMETHASONE ACETATE:Bone marrow conditioning regimen:0.48), (DILTIAZEM HYDROCHLORIDE:Bone marrow conditioning regimen:0.11), (DIVALPROEX SODIUM:Bone marrow conditioning regimen:0.76), (DOXYCYCLINE:Bone marrow conditioning regimen:0.07), (ELOTUZUMAB:Bone marrow conditioning regimen:0.13), (ESZOPICLONE:Bone marrow conditioning regimen:0.02), (FERROUS SULFATE:Bone marrow conditioning regimen:0.09), (FERROUS SULFATE\FERROUS SULFATE, DRIED:Bone marrow conditioning regimen:0.2), (FLUCYTOSINE:Bone marrow conditioning regimen:0.39), (FOSFOMYCIN:Bone marrow conditioning regimen:0.38), (GUSPERIMUS TRIHYDROCHLORIDE:Bone marrow conditioning regimen:0.91), (IBUPROFEN:Bone marrow conditioning regimen:0.32), (IDELALISIB:Bone marrow conditioning regimen:0.5), (INTERFERON GAMMA-1B:Bone marrow conditioning regimen:0.22), (LACTOFERRIN:Bone marrow conditioning regimen:0.11), (LIDOCAINE\PRILOCAINE:Bone marrow conditioning regimen:0.3), (LYMPHOCYTES:Bone marrow conditioning regimen:0.8), (MELATONIN:Bone marrow conditioning regimen:0.01), (METHENOLONE ENANTHATE:Bone marrow conditioning regimen:0.94), (METHOTREXATE\METHOTREXATE SODIUM:Bone marrow conditioning regimen:0.41), (MOGAMULIZUMAB:Bone marrow conditioning regimen:0.81), (NELARABINE:Bone marrow conditioning regimen:0.61), (NOREPINEPHRINE:Bone marrow conditioning regimen:0.55), (OXCARBAZEPINE:Bone marrow conditioning regimen:0.77), (PANTOPRAZOLE:Bone marrow conditioning regimen:0.45), (POTASSIUM:Bone marrow conditioning regimen:0.16), (PREDNISOLONE SODIUM PHOSPHATE:Bone marrow conditioning regimen:0.76), (PYRAZINAMIDE:Bone marrow conditioning regimen:0.66), (SULFAMERAZINE\TRIMETHOPRIM:Bone marrow conditioning regimen:0.42), (TAZOBACTAM:Bone marrow conditioning regimen:0.18), (TENIPOSIDE:Bone marrow conditioning regimen:0.01), (TISAGENLECLEUCEL:Bone marrow conditioning regimen:0.47), (TOPIRAMATE:Bone marrow conditioning regimen:0.34), (TRANEXAMIC ACID:Bone marrow conditioning regimen:0.14), (TRAZODONE HYDROCHLORIDE:Bone marrow conditioning regimen:0.28), (VERAPAMIL HYDROCHLORIDE:Bone marrow conditioning regimen:0.13), (VINDESINE SULFATE:Bone marrow conditioning regimen:0.71), (WARFARIN SODIUM:Bone marrow conditioning regimen:0.25), (IDARUBICIN:Bone marrow failure:0.01), (IMATINIB MESYLATE:Bone marrow failure:0.44), (IRINOTECAN:Bone marrow failure:0.82), (LEVOLEUCOVORIN CALCIUM:Bone marrow failure:0.17), (RAMIPRIL:Bone marrow failure:0.04), (THALIDOMIDE:Bone marrow failure:0.13), (BLINATUMOMAB:Bone marrow transplant:0.03), (IRINOTECAN:Bone neoplasm:0.04), (ACARBOSE:Bone pain:0.38), (ACETAMINOPHEN\PROPOXYPHENE NAPSYLATE:Bone pain:0.11), (ACETAZOLAMIDE:Bone pain:0.12), (AGALSIDASE BETA:Bone pain:0.37), (ALCOHOL:Bone pain:0.86), (ALDESLEUKIN:Bone pain:0.14), (ALUMINUM HYDROXIDE\DIMETHICONE\MAGNESIUM HYDROXIDE:Bone pain:0.48), (AMILORIDE HYDROCHLORIDE\HYDROCHLOROTHIAZIDE:Bone pain:0.11), (AMLODIPINE\PERINDOPRIL:Bone pain:0.08),(ARMODAFINIL:Bone pain:0.86), (ASPIRIN\MAGNESIUM OXIDE:Bone pain:0.23), (AZITHROMYCIN DIHYDRATE:Bone pain:0.31), (BENZONATATE:Bone pain:0.38), (BUDESONIDE\FORMOTEROL FUMARATE:Bone pain:0.43), (BUMETANIDE:Bone pain:1), (BUPRENORPHINE HYDROCHLORIDE\NALOXONE HYDROCHLORIDE:Bone pain:0.57), (CALCITONIN:Bone pain:0.38), (CANAGLIFLOZIN\METFORMIN HYDROCHLORIDE:Bone pain:0.06), (CARVEDILOL PHOSPHATE:Bone pain:0.69), (CEFEPIME\CEFEPIME HYDROCHLORIDE:Bone pain:0.01), (CEFUROXIME AXETIL:Bone pain:0.11), (CETUXIMAB:Bone pain:0.43), (CHLORTHALIDONE:Bone pain:0.85), (CICLESONIDE:Bone pain:0.24), (CILASTATIN SODIUM\IMIPENEM:Bone pain:1.43), (CIMETIDINE:Bone pain:1.18), (CLOZAPINE:Bone pain:1.24), (COD LIVER OIL:Bone pain:0.03), (DABIGATRAN:Bone pain:0.19), (DABRAFENIB MESYLATE:Bone pain:0.23), (DACLIZUMAB:Bone pain:0.64), (DALFAMPRIDINE:Bone pain:0.57), (DANAZOL:Bone pain:0.08), (DEFLAZACORT:Bone pain:0.16), (DEVICE:Bone pain:0.2), (DOCUSATE CALCIUM:Bone pain:0.49), (DOFETILIDE:Bone pain:0.58), (DOLASETRON MESYLATE:Bone pain:0.38), (DOXEPIN HYDROCHLORIDE:Bone pain:0.03), (DOXYCYCLINE HYCLATE:Bone pain:0.05), (EDOXABAN TOSYLATE:Bone pain:0.81), (EPLERENONE:Bone pain:0.42), (ERIBULIN:Bone pain:0.49), (ERYTHROMYCIN:Bone pain:0.56), (ESTROGENS, CONJUGATED\MEDROXYPROGESTERONE ACETATE:Bone pain:0.56), (EVOLOCUMAB:Bone pain:0.48), (FENOFIBRIC ACID:Bone pain:0.19), (FERROUS SULFATE\GLYCINE:Bone pain:0.15), (FESOTERODINE FUMARATE:Bone pain:1.03), (FLUOCINONIDE:Bone pain:0.08), (GEFITINIB:Bone pain:0.59), (GENTAMICIN:Bone pain:0.43), (GLYCOPYRRONIUM\INDACATEROL:Bone pain:0.91), (HEPARIN CALCIUM:Bone pain:0.81), (HERBALS\SAW PALMETTO:Bone pain:0.02), (HYDROCHLOROTHIAZIDE\TELMISARTAN:Bone pain:0.61), (HYDROXYCHLOROQUINE DIPHOSPHATE:Bone pain:0.18), (HYDROXYZINE PAMOATE:Bone pain:1.07), (HYOSCYAMINE:Bone pain:0.31), (IBRITUMOMAB TIUXETAN:Bone pain:0.34), (IMIPENEM:Bone pain:0.11), (IMIPRAMINE HYDROCHLORIDE:Bone pain:0.76), (INFLIXIMAB-DYYB:Bone pain:0.02), (INTERFERON ALFA-2A:Bone pain:0.54), (INTERFERON ALFA-2B:Bone pain:0.44), (IRINOTECAN:Bone pain:0.17), (LEDIPASVIR\SOFOSBUVIR:Bone pain:0.42), (MECLIZINE\MECLIZINE HYDROCHLORIDE:Bone pain:0.33), (METFORMIN HYDROCHLORIDE\VILDAGLIPTIN:Bone pain:0.58), (METHENAMINE HIPPURATE:Bone pain:0.87), (METHIMAZOLE:Bone pain:0.47), (METHYLPREDNISOLONE ACETATE:Bone pain:1.21), (METOLAZONE:Bone pain:0.15), (MODAFINIL:Bone pain:0.29), (MUPIROCIN:Bone pain:0.15), (MYCOPHENOLATE MOFETIL\MYCOPHENOLATE MOFETIL HYDROCHLORIDE:Bone pain:0.2), (NADOLOL:Bone pain:0.19), (NATEGLINIDE:Bone pain:0.12), (NICARDIPINE:Bone pain:0.12), (NINTEDANIB:Bone pain:0.4), (OLODATEROL HYDROCHLORIDE\TIOTROPIUM BROMIDE MONOHYDRATE:Bone pain:0.11), (OMEGA-3-ACID ETHYL ESTERS:Bone pain:1.04), (PANTOPRAZOLE MAGNESIUM:Bone pain:1.25), (PEGINTERFERON BETA-1A:Bone pain:0.07), (PENICILLIN:Bone pain:1.08), (PHENOBARBITAL:Bone pain:1.27), (PHENYTOIN SODIUM:Bone pain:0.63), (PHLOROGLUCINOL1,3,5-TRIMETHOXYBENZENE:Bone pain:0.01), (PYRIDOXINE:Bone pain:0.51), (RADIATION THERAPY:Bone pain:0.87), (RAMUCIRUMAB:Bone pain:0.61), (RIVASTIGMINE TARTRATE:Bone pain:0.07), (RIZATRIPTAN BENZOATE:Bone pain:0.62), (ROFLUMILAST:Bone pain:0.64), (ROPINIROLE:Bone pain:0.6), (SACCHAROMYCES CEREVISIAE:Bone pain:0.05), (SACUBITRIL\VALSARTAN:Bone pain:1.09), (TAMOXIFEN CITRATE:Bone pain:0.53), (TAZOBACTAM:Bone pain:0.1), (TERAZOSIN HYDROCHLORIDE:Bone pain:0.14), (TERIFLUNOMIDE:Bone pain:0.19), (TIMOLOL:Bone pain:0.68), (TRANDOLAPRIL:Bone pain:0.01), (VALGANCICLOVIR HYDROCHLORIDE:Bone pain:0.69), (VANCOMYCIN HYDROCHLORIDE:Bone pain:0.77), (VARENICLINE TARTRATE:Bone pain:0.88), (VITAMIN A:Bone pain:0.29), (ZINC:Bone pain:0.25), (ZIV-AFLIBERCEPT:Bone pain:0.05), (ZONISAMIDE:Bone pain:0.29), (ATORVASTATIN CALCIUM:Borderline personality disorder:0.15), (BUPRENORPHINE HYDROCHLORIDE:Borderline personality disorder:0.34), (PAROXETINE\PAROXETINE HYDROCHLORIDE:Borderline personality disorder:0.09), (ACETAMINOPHEN\BUTALBITAL\CAFFEINE:Bowel movement irregularity:0.26), (AMIODARONE:Bowel movement irregularity:0.61), (BISOPROLOL FUMARATE:Bowel movement irregularity:0.47), (BUPRENORPHINE HYDROCHLORIDE\NALOXONE HYDROCHLORIDE:Bowel movement irregularity:0.06), (BUPROPION HYDROCHLORIDE:Bowel movement irregularity:1.43), (CANDESARTAN:Bowel movement irregularity:0.34), (CARVEDILOL PHOSPHATE:Bowel movement irregularity:0.08), (CLOTRIMAZOLE:Bowel movement irregularity:0.01), (CORTISONE\HYDROCORTISONE:Bowel movement irregularity:0.25), (DASATINIB:Bowel movement irregularity:0.33), (DEVICE:Bowel movement irregularity:0.11), (DOXAZOSIN MESYLATE:Bowel movement irregularity:0.08), (ELETRIPTAN HYDROBROMIDE:Bowel movement irregularity:0.36), (ESZOPICLONE:Bowel movement irregularity:0.74), (EZETIMIBE\SIMVASTATIN:Bowel movement irregularity:0.07), (FLECAINIDE:Bowel movement irregularity:0.03), (FLUTICASONE\FLUTICASONE PROPIONATE:Bowel movement irregularity:0.01), (HEPARIN SODIUM:Bowel movement irregularity:0.19), (HYDRALAZINE HYDROCHLORIDE:Bowel movement irregularity:0.07), (HYDROMORPHONE:Bowel movement irregularity:0.08), (HYDROXYZINE PAMOATE:Bowel movement irregularity:0.22), (INDOMETHACIN:Bowel movement irregularity:0.19), (INSULIN DETEMIR:Bowel movement irregularity:0.16), (LEDIPASVIR\SOFOSBUVIR:Bowel movement irregularity:0.4), (LISDEXAMFETAMINE DIMESYLATE:Bowel movement irregularity:0.43), (METAXALONE:Bowel movement irregularity:0.7), (METHYLPREDNISOLONE SODIUM SUCCINATE:Bowel movement irregularity:0.58), (MOXIFLOXACIN HYDROCHLORIDE:Bowel movement irregularity:0.11), (NEBIVOLOL:Bowel movement irregularity:0.1), (NEBIVOLOL HYDROCHLORIDE:Bowel movement irregularity:0.19), (NILOTINIB:Bowel movement irregularity:0.04), (OMEGA-3-ACID ETHYL ESTERS:Bowel movement irregularity:0.25), (PANTOPRAZOLE MAGNESIUM:Bowel movement irregularity:0.18), (PAROXETINE:Bowel movement irregularity:0.35), (PAZOPANIB HYDROCHLORIDE:Bowel movement irregularity:0.21), (PHENYTOIN:Bowel movement irregularity:0.78), (PRAMIPEXOLE:Bowel movement irregularity:0.11), (PRAMIPEXOLE DIHYDROCHLORIDE:Bowel movement irregularity:0.59), (QUETIAPINE:Bowel movement irregularity:0.42), (RALOXIFENE HYDROCHLORIDE:Bowel movement irregularity:0.03), (RANOLAZINE:Bowel movement irregularity:0.59), (RISEDRONATE SODIUM:Bowel movement irregularity:0.01), (ROPINIROLE HYDROCHLORIDE:Bowel movement irregularity:0.13), (SACUBITRIL\VALSARTAN:Bowel movement irregularity:0.41), (SUMATRIPTAN\SUMATRIPTAN SUCCINATE:Bowel movement irregularity:0.37), (TERIFLUNOMIDE:Bowel movement irregularity:0.73), (VITAMIN B:Bowel movement irregularity:0.5), (ZINC:Bowel movement irregularity:0.03), (ACETAMINOPHEN\HYDROCODONE BITARTRATE:Bowel preparation:0.04), (AMIODARONE HYDROCHLORIDE:Bowel preparation:0.13), (BUPROPION HYDROCHLORIDE:Bowel preparation:0.55), (CERTOLIZUMAB PEGOL:Bowel preparation:0.21), (DICLOFENAC SODIUM:Bowel preparation:0.13), (ESZOPICLONE:Bowel preparation:0), (EVOLOCUMAB:Bowel preparation:0.48), (FERROUS SULFATE\FERROUS SULFATE, DRIED:Bowel preparation:0.37), (IBRUTINIB:Bowel preparation:0.78), (INSULIN HUMAN:Bowel preparation:0.17), (NILOTINIB:Bowel preparation:0.01), (PANTOPRAZOLE:Bowel preparation:0.49), (POTASSIUM:Bowel preparation:0.58), (RANOLAZINE:Bowel preparation:0.15), (TERIFLUNOMIDE:Bowel preparation:0.42), (TIOTROPIUM BROMIDE MONOHYDRATE:Bowel preparation:0.01), (ATRACURIUM:Brady cardia:0.55), (CALCIUM CARBONATE\CHOLECALCIFEROL:Bradycardia:0.04), (IBRUTINIB:Brady cardia:0.36), (ISO FLURANE:Bradycardia:0.85), (LOVASTATIN:Bradycardia:0.2), (OMEPRAZOLE MAGNESIUM:Bradycardia:0.14), (PYRAZINAMIDE:Bradycardia:0.62), (SUCCINYLCHOLIN E CHLORIDE:Bradycardia:0.24), (THIOPENTAL:Bradycardia:0.06), (BUDESONIDE:Brain neoplasm:0.29), (CALCIUM CARBONATE\CHOLECALCIFEROL:Brain neoplasm:0.31), (CANDESARTAN:Brain neoplasm:0.1), (CERTOLIZUMAB PEGOL:Brain neoplasm:0.35), (CLOZAPINE:Brain neoplasm:0.21), (DALFAMPRIDINE:Brain neoplasm:0.12), (DICLOFENAC SODIUM:Brain neoplasm:0.33), (ENALAPRIL MALEATE:Brain neoplasm:0.01), (ENOXAPARIN:Brain neoplasm:0.19), (EPIRUBICIN:Brain neoplasm:0.45), (HYDROCHLOROTHIAZIDE\LISINOPRIL:Brain neoplasm:0.13), (IBRUTINIB:Brain neoplasm:0.34), (LEUCOVORIN CALCIUM:Brain neoplasm:0.57), (MELOXICAM:Brain neoplasm:0.21), (METRONIDAZOLE:Brain neoplasm:0.06), (MORPHINE:Brain neoplasm:0.31), (NILOTINIB:Brain neoplasm:0.54), (OLANZAPINE:Brain neoplasm:0.41), (OXYGEN:Brain neoplasm:0), (REGORAFENIB:Brain neoplasm:0.11), (SPIRONOLACTONE:Brain neoplasm:0.66), (TERIFLUNOMIDE:Brain neoplasm:0.44), (TOCILIZUMAB:Brain neoplasm:0.08), (VARENICLINE TARTRATE:Brain neoplasm:0.06), (BRIVARACETAM:Brain neoplasm benign:0.1), (AMIODARONE HYDROCHLORIDE:Brain neoplasm malignant:0.12),(ARMODAFINIL:Brain neoplasm malignant:0.04), (ATROPINE SULFATE\DIPHENOXYLATE HYDROCHLORIDE:Brain neoplasm malignant:0.25), (BICALUTAMIDE:Brain neoplasm malignant:0.43), (BISOPROLOL FUMARATE:Brain neoplasm malignant:0.05), (BUMETANIDE:Brain neoplasm malignant:0.34), (CALCIUM ACETATE:Brain neoplasm malignant:0.09), (CALCIUM CARBONATE\CHOLECALCIFEROL:Brain neoplasm malignant:0.89), (CANDESARTAN:Brain neoplasm malignant:0.76), (CANDESARTAN CILEXETIL\HYDROCHLOROTHIAZIDE:Brain neoplasm malignant:0.2), (CARFILZOMIB:Brain neoplasm malignant:0.27), (CERTOLIZUMAB PEGOL:Brain neoplasm malignant:0.58), (CHOLESTYRAMINE:Brain neoplasm malignant:0.81), (CYCLOBENZAPRINE HYDROCHLORIDE:Brain neoplasm malignant:0.4), (DABRAFENIB:Brain neoplasm malignant:0.16), (DEVICE:Brain neoplasm malignant:0.27), (DICLOFENAC:Brain neoplasm malignant:0.13), (DICLOFENAC SODIUM:Brain neoplasm malignant:0.11), (DILTIAZEM:Brain neoplasm malignant:0.22), (ELETRIPTAN HYDROBROMIDE:Brain neoplasm malignant:0.08), (ERIBULIN:Brain neoplasm malignant:0.14), (ESTROGENS, CONJUGATED:Brain neoplasm malignant:0.31), (EVOLOCUMAB:Brain neoplasm malignant:0.22), (GRANULOCYTE COLONY-STIMULATING FACTOR NOS:Brain neoplasm malignant:0.42), (HYDROCHLOROTHIAZIDE\LISINOPRIL:Brain neoplasm malignant:0.56), (IBRUTINIB:Brain neoplasm malignant:1.09), (LERCANIDIPINE:Brain neoplasm malignant:0.08), (LEUCOVORIN CALCIUM:Brain neoplasm malignant:0.81), (LEVOLEUCOVORIN CALCIUM:Brain neoplasm malignant:0.42), (LISDEXAMFETAMINE DIMESYLATE:Brain neoplasm malignant:0.01), (MELOXICAM:Brain neoplasm malignant:0.79), (METAXALONE:Brain neoplasm malignant:0.39), (MOXIFLOXACIN HYDROCHLORIDE:Brain neoplasm malignant:0.57), (NAPROXEN:Brain neoplasm malignant:0.85), (NEBIVOLOL HYDROCHLORIDE:Brain neoplasm malignant:0.12), (NILOTINIB:Brain neoplasm malignant:1.38), (OLAPARIB:Brain neoplasm malignant:0.53), (OXYGEN:Brain neoplasm malignant:0.19), (PEMETREXED:Brain neoplasm malignant:0.27), (PENICILLIN:Brain neoplasm malignant:0.25), (PRAMIPEXOLE DIHYDROCHLORIDE:Brain neoplasm malignant:0.05), (PRIMIDONE:Brain neoplasm malignant:0.26), (QUETIAPINE:Brain neoplasm malignant:0.01), (RABEPRAZOLE:Brain neoplasm malignant:0), (ROFLUMILAST:Brain neoplasm malignant:0.06), (SACUBITRIL\VALSARTAN:Brain neoplasm malignant:0.09), (SODIUM OXYBATE:Brain neoplasm malignant:0.14), (SOTALOL:Brain neoplasm malignant:0.08), (SPIRONOLACTONE:Brain neoplasm malignant:1.13), (TIZANIDINE:Brain neoplasm malignant:0.06), (TIZANIDINE HYDROCHLORIDE:Brain neoplasm malignant:0.18), (TOCILIZUMAB:Brain neoplasm malignant:0.19), (TRAMETINIB:Brain neoplasm malignant:0.11), (VINORELBINE\VINORELBINE TARTRATE:Brain neoplasm malignant:0.15), (AMIODARONE:Brain oedema:0.13), (ANASTROZOLE:Brain oedema:0.58), (BUMETANIDE:Brain oedema:0.14), (BUPROPION HYDROCHLORIDE:Brain oedema:0.1), (CEPHALEXIN:Brain oedema:0.2), (CLINDAMYCIN\CLINDAMYCIN PHOSPHATE:Brain oedema:0.08), (CLOZAPINE:Brain oedema:0.23), (EXEMESTANE:Brain oedema:0.04), (FISH OIL:Brain oedema:0.33), (LITHIUM CARBONATE:Brain oedema:0.04), (METOPROLOL TARTRATE:Brain oedema:0.05), (NAPROXEN:Brain oedema:0.59), (NEBIVOLOL:Brain oedema:0.14), (NILOTINIB:Brain oedema:0.87), (OMEPRAZOLE MAGNESIUM:Brain oedema:0.36), (POLYETHYLENE GLYCOLS:Brain oedema:0.11), (PRIMIDONE:Brain oedema:0.05), (SACUBITRIL\VALSARTAN:Brain oedema:0.29), (TELMISARTAN:Brain oedema:0.06), (THALIDOMIDE:Brain oedema:0.77), (VENLAFAXINE HYDROCHLORIDE:Brain oedema:0.42), (ZOLPIDEM\ZOLPIDEM TARTRATE:Brain oedema:0.29), (.ALPHA.1-PROTEINASE INHIBITOR HUMAN:Breakthrough pain:0.16), (ACARBOSE:Breakthrough pain:0.19), (ACETAZOLAMIDE:Breakthrough pain:0.59), (ADO-TRASTUZUMAB EMTANSINE:Breakthrough pain:0.81), (ALBIGLUTIDE:Breakthrough pain:0.03), (ALEMTUZUMAB:Breakthrough pain:0.12), (ALENDRONIC ACID:Breakthrough pain:0.55), (AMANTADINE HYDROCHLORIDE:Breakthrough pain:0.07), (AMISULPRIDE:Breakthrough pain:0.3), (AMLODIPINE\VALSARTAN:Breakthrough pain:0.06), (AZITHROMYCIN ANHYDROUS:Breakthrough pain:0.82), (BENAZEPRIL\BENAZEPRIL HYDROCHLORIDE:Breakthrough pain:0.57), (BIMATOPROST:Breakthrough pain:0.11), (BROMAZEPAM:Breakthrough pain:0.26), (BUDESONIDE\FORMOTEROL FUMARATE:Breakthrough pain:0.14), (CALCITONIN:Breakthrough pain:0.23), (CANAGLIFLOZIN\METFORMIN HYDROCHLORIDE:Breakthrough pain:0.14), (CANAKINUMAB:Breakthrough pain:0.07), (CANDESARTAN:Breakthrough pain:1.47), (CANDESARTAN CILEXETIL\HYDROCHLOROTHIAZIDE:Breakthrough pain:0.76), (CAPTOPRIL:Breakthrough pain:0.19), (CARVEDILOL PHOSPHATE:Breakthrough pain:1.15), (CHLORDIAZEPOXIDE HYDROCHLORIDE\CLIDINIUM BROMIDE:Breakthrough pain:0.06), (CHOLESTYRAMINE:Breakthrough pain:1.45), (CLOZAPINE:Breakthrough pain:1.11), (COD LIVER OIL:Breakthrough pain:0.13), (COLCHICINE:Breakthrough pain:0.92), (DACLIZUMAB:Breakthrough pain:0.54), (DALTEPARIN:Breakthrough pain:0.19), (DANAZOL:Breakthrough pain:0.35), (DANTROLENE SODIUM:Breakthrough pain:0.1), (DAPAGLIFLOZIN PROPANEDIOL:Breakthrough pain:0.2), (DASATINIB:Breakthrough pain:1.29), (DEFLAZACORT:Breakthrough pain:0.09), (DESLORATADINE:Breakthrough pain:0.45), (DEXAMETHASONE ACETATE:Breakthrough pain:0.06), (DIPYRONE:Breakthrough pain:0.33), (DOXAZOSIN MESYLATE:Breakthrough pain:0.86), (DOXYLAMINE SUCCINATE:Breakthrough pain:0.01), (DULAGLUTIDE:Breakthrough pain:0.04), (EDOXABAN TOSYLATE:Breakthrough pain:0.61), (ELETRIPTAN HYDROBROMIDE:Breakthrough pain:1.36), (EMPAGLIFLOZIN\LINAGLIPTIN:Breakthrough pain:0.28), (ESTROGENS:Breakthrough pain:0.11), (ESTRONE\SUS SCROFA OVARY:Breakthrough pain:0.02), (EVOLOCUMAB:Breakthrough pain:1.09), (EZETIMIBE\SIMVASTATIN:Breakthrough pain:0.69), (FEXOFENADINE\PSEUDOEPHEDRINE:Breakthrough pain:0.09), (FLUTICASONE FUROATE:Breakthrough pain:0.5), (FLUVOXAMINE MALEATE:Breakthrough pain:0.14), (FORMOTEROL FUMARATE:Breakthrough pain:0.15), (FUSIDIC ACID:Breakthrough pain:0.38), (GLYCOPYRRONIUM\INDACATEROL:Breakthrough pain:0.07), (HYALURONATE SODIUM:Breakthrough pain:0.38), (HYDROXYCHLOROQUINE DIPHOSPHATE:Breakthrough pain:0.31), (ILOPROST:Breakthrough pain:0.15), (INDAPAMIDE:Breakthrough pain:0.52), (INDAPAMIDE\PERINDOPRIL:Breakthrough pain:0.18), (IXAZOMIB:Breakthrough pain:0.22), (LABETALOL\LABETALOL HYDROCHLORIDE:Breakthrough pain:1.55), (LACOSAMIDE:Breakthrough pain:0.1), (LACTASE:Breakthrough pain:0.06), (LAPATINIB DITOSYLATE:Breakthrough pain:0.15), (LEVOMEFOLATE CALCIUM:Breakthrough pain:0.02), (LOMITAPIDE MESYLATE:Breakthrough pain:0.35), (MEDROXYPROGESTERONE ACETATE:Breakthrough pain:0.42), (METHENAMINE HIPPURATE:Breakthrough pain:1.3), (METHIMAZOLE:Breakthrough pain:0.1), (METHYLPREDNISOLONE ACETATE:Breakthrough pain:0.9), (MIDODRINE HYDROCHLORIDE:Breakthrough pain:0.07), (MUPIROCIN\MUPIROCIN CALCIUM:Breakthrough pain:0.38), (NILOTINIB:Breakthrough pain:1.59), (NINTEDANIB:Breakthrough pain:0.49), (NIRAPARIB:Breakthrough pain:0.29), (OLAPARIB:Breakthrough pain:0.45), (OLODATEROL HYDROCHLORIDE\TIOTROPIUM BROMIDE MONOHYDRATE:Breakthrough pain:0.23), (OSELTAMIVIR PHOSPHATE:Breakthrough pain:0.02), (PARATHYROID HORMONE:Breakthrough pain:0.08), (PATIROMER:Breakthrough pain:0.2), (PEGINTERFERON BETA-1A:Breakthrough pain:0.73), (PEMETREXED:Breakthrough pain:0.26), (PENICILLIN:Breakthrough pain:1), (PERINDOPRIL:Breakthrough pain:0.52), (PERTUZUMAB:Breakthrough pain:0.06), (PITAVASTATIN CALCIUM:Breakthrough pain:0.65), (PONATINIB:Breakthrough pain:0.71), (PROPAFENONE HYDROCHLORIDE:Breakthrough pain:0.1), (RADIATION THERAPY:Breakthrough pain:0.52), (RALOXIFENE HYDROCHLORIDE:Breakthrough pain:1.03), (RAMUCIRUMAB:Breakthrough pain:0.46), (RIVASTIGMINE:Breakthrough pain:0.15), (RIZATRIPTAN:Breakthrough pain:0.05), (SACCHAROMYCES CEREVISIAE:Breakthrough pain:0.05), (TEDUGLUTIDE\WATER:Breakthrough pain:0.11), (TERBINAFINE HYDROCHLORIDE:Breakthrough pain:0.25), (TETRACYCLINE:Breakthrough pain:0.72), (TIOTROPIUM:Breakthrough pain:0.29), (VALGANCICLOVIR HYDROCHLORIDE:Breakthrough pain:0.2), (ZAFIRLUKAST:Breakthrough pain:0.01), (ZINC:Breakthrough pain:0.44), (PYRAZINAMIDE:Breast abscess:0.54), (.ALPHA.1-PROTEINASE INHIBITOR HUMAN:Breast cancer:0.53), (ABIRATERONE:Breast cancer:0.94), (ABOBOTULINUMTOXINA:Breast cancer:0.54), (ACITRETIN:Breast cancer:0.87), (ACTIVATED CHARCOAL:Breast cancer:0.76), (ADENINE:Breast cancer:0.17), (AGALSIDASE BETA:Breast cancer:1.44), (ALECTINIB:Breast cancer:0.27), (ALEMTUZUMAB:Breast cancer:0.76), (ALFUZOSIN:Breast cancer:1.17), (ALFUZOSIN HYDROCHLORIDE:Breast cancer:1.59), (ALUMINUM HYDROXIDE\MAGNESIUM OXIDE\OXETHAZAINE:Breast cancer:0.13), (AMBROXOL\CLENBUTEROL:Breast cancer:0.15), (AMIKACIN:Breast cancer:0.13), (AMINO ACIDS\DEXTROSE\ELECTROLYTES NOS:Breast cancer:0.61), (AMINO ACIDS\DEXTROSE\ELECTROLYTES NOS\SOYBEAN OIL:Breast cancer:0.01), (AMPICILLIN SODIUM:Breast cancer:0.84), (ANAGRELIDE HYDROCHLORIDE:Breast cancer:0.43), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Breast cancer:0.25), (ASPARAGINASE:Breast cancer:0.1), (ASPIRIN\CLOPIDOGREL:Breast cancer:0.17), (ASPIRIN\DIHYDROXYALUMINUM AMINOACETATE ANHYDROUS\MAGNESIUM CARBONATE:Breast cancer:0.53), (ATOVAQUONE:Breast cancer:0.47), (AZITHROMYCIN OR METHOTREXATE SODIUM:Breast cancer:0.33), (BALSALAZIDE DISODIUM:Breast cancer:0.33), (BAZEDOXIFENE ACETATE\ESTROGENS, CONJUGATED:Breast cancer:0.11), (BECLOMETHASONE DIPROPIONATE\FORMOTEROL:Breast cancer:0.25), (BENSERAZIDE\LEVODOPA:Breast cancer:0.2), (BENZOCAINE:Breast cancer:0.35), (BENZTROPINE MESYLATE:Breast cancer:0.52), (BETAMETHASONE DIPROPIONATE\CALCIPOTRIENE:Breast cancer:0.43), (BETAMETHASONE\CALCIPOTRIENE:Breast cancer:0.83), (BLINATUMOMAB:Breast cancer:1.26), (BOCEPREVIR:Breast cancer:0.51), (BOCOCIZUMAB:Breast cancer:0.12), (BOSUTINIB:Breast cancer:0.27), (BOTULINUM TOXIN TYPE A:Breast cancer:0.81), (BRINZOLAMIDE\TIMOLOL MALEATE:Breast cancer:0.34), (BUTALBITAL:Breast cancer:0.26), (CALCIUM CARBONATE\ERGOCALCIFEROL\RETINOL:Breast cancer:0.51), (CALCIUM PANTOTHENATE\CYANOCOBALAMIN\NIACINAMIDE\PYRIDOXINE HYDROCHLORIDE\RIBOFLAVIN\THIAMINE MONONITRATE:Breast cancer:0.66), (CALCIUM\MAGNESIUM\ZINC:Breast cancer:0.94), (CANAKINUMAB:Breast cancer:1.08), (CAPECITABINE\OXALIPLATIN:Breast cancer:0.14), (CARBASALATE:Breast cancer:0.28), (CARTEOLOL HYDROCHLORIDE:Breast cancer:0.24), (CEFOTAXIME SODIUM:Breast cancer:1.24), (CEFOTAXIME\CEFOTAXIME SODIUM:Breast cancer:0.17), (CEFTAZIDIME SODIUM:Breast cancer:0.16), (CEPHARANTHINE:Breast cancer:0.23), (CERITINIB:Breast cancer:1.75), (CHIMAPHILA UMBELLATA\MANGANESE CHLORIDE\POPULUS TREMULOIDES\SODIUM TAUROCHOLATE:Breast cancer:0.05), (CHLOROTHIAZIDE:Breast cancer:0.08), (CHOLESTEROL:Breast cancer:0.04), (CHROMIC CHLORIDE\CHROMIUM:Breast cancer:0.14), (CLADRIBINE:Breast cancer:0.23), (CLOMIPRAMINE:Breast cancer:0.06), (COLCHICINE\DICYCLOMINE HYDROCHLORIDE:Breast cancer:0.08), (CORTICOTROPIN:Breast cancer:0.93), (CYANOCOBALAMIN\FOLIC ACID\PYRIDOXINE:Breast cancer:0.23), (CYPROTERONE:Breast cancer:0.13), (CYPROTERONE ACETATE:Breast cancer:0.55), (DACLATASVIR DIHYDROCHLORIDE:Breast cancer:0.09), (DACLIZUMAB:Breast cancer:1.19), (DANAZOL:Breast cancer:1.36), (DANTROLENE SODIUM:Breast cancer:0.56), (DAPAGLIFLOZIN PROPANEDIOL\METFORMIN HYDROCHLORIDE:Breast cancer:0.24), (DASABUVIR\OMBITASVIR\PARITAPREVIR\RITONAVIR:Breast cancer:0.62), (DEFERIPRONE:Breast cancer:0.38), (DEGARELIX:Breast cancer:0.82), (DEGARELIX ACETATE:Breast cancer:0.51), (DEXTROMETHORPHAN HYDROBROMIDE\QUINIDINE SULFATE:Breast cancer:0.01), (DICLOXACILLIN:Breast cancer:0.07), (DILAZEP:Breast cancer:0.11), (DIPYRONE\PROMETHAZINE HYDROCHLORIDE:Breast cancer:0.37), (DOCUSATE SODIUM\SENNOSIDES A AND B:Breast cancer:0.1), (DOCUSATE\SENNOSIDES:Breast cancer:0.31), (DROXIDOPA:Breast cancer:0.36), (DUTASTERIDE:Breast cancer:3.09), (DUTASTERIDE\TAMSULOSIN HYDROCHLORIDE:Breast cancer:0.86), (ECONAZOLE:Breast cancer:1.38), (EFINACONAZOLE:Breast cancer:0.07), (ELOTUZUMAB:Breast cancer:1.32), (EMPAGLIFLOZIN\LINAGLIPTIN:Breast cancer:1.22), (EPACADOSTAT:Breast cancer:0.03), (EPERISONE HYDROCHLORIDE:Breast cancer:0.2), (ERTAPENEM SODIUM:Breast cancer:0.54), (ESTRADIOL\NORETHINDRONE ACETATE:Breast cancer:0.39), (ESTROGENS, CONJUGATED\MEDROXYPROGESTERONE ACETATE:Breast cancer:1.65), (ETHINYL ESTRADIOL\NORETHINDRONE:Breast cancer:0.15), (ETHINYL ESTRADIOL\NORGESTREL:Breast cancer:0.45), (ETRETINATE:Breast cancer:1.54), (FISH OIL\TOCOPHEROL:Breast cancer:0.1), (FLUNARIZINE HYDROCHLORIDE:Breast cancer:0.22), (FLUOCINOLONE ACETONIDE:Breast cancer:0.58), (FLUOROURACIL\IRINOTECAN\LEUCOVORIN:Breast cancer:0.73), (FLUOROURACIL\LEUCOVORIN CALCIUM\OXALIPLATIN:Breast cancer:1.19), (FLUTAMIDE:Breast cancer:0.93), (FORMOTEROL FUMARATE DIHYDRATE:Breast cancer:0.05), (FORMOTEROL FUMARATE\MOMETASONE FUROATE:Breast cancer:1.05), (FOSPHENYTOIN\FOSPHENYTOIN SODIUM:Breast cancer:0.03), (GADOTERATE MEGLUMINE:Breast cancer:0.15), (GUANFACINE:Breast cancer:0.79), (HERBALS\SAW PALMETTO:Breast cancer:1.21), (HYDROCHLOROTHIAZIDE\QUINAPRIL HYDROCHLORIDE:Breast cancer:0.32), (HYDROXYCHLOROQUINE DIPHOSPHATE:Breast cancer:0.85), (IBRITUMOMAB TIUXETAN:Breast cancer:0.47), (IMIGLUCERASE:Breast cancer:0.95), (INFLIXIMAB-DYYB:Breast cancer:0.28), (INOTUZUMAB OZOGAMICIN:Breast cancer:0.25), (INSULIN PORK\INSULIN PURIFIED PORK:Breast cancer:0.05), (INTERFERON:Breast cancer:1.49), (INTERFERON ALFA-2A OR INTERFERON ALFA-2B:Breast cancer:0.51), (IRON\POLYSACCHARIDES:Breast cancer:0.63), (KETAMINE HYDROCHLORIDE:Breast cancer:0.1), (LACTOBACILLUS ACIDOPHILUS\LACTOBACILLUS DELBRUECKII SUBSP. BULGARICUS:Breast cancer:0.44), (LANREOTIDE:Breast cancer:0.71), (LEVOMILNACIPRAN HYDROCHLORIDE:Breast cancer:0.03), (LINSIDOMINE:Breast cancer:0.54), (LOPRAZOLAM:Breast cancer:0.03), (LURASIDONE HYDROCHLORIDE:Breast cancer:0.71), (LUTETIUM LU-177:Breast cancer:0.71), (MAGNESIUM ASPARTATE HYDROCHLORIDE:Breast cancer:0.16), (MAGNESIUM PIDOLATE:Breast cancer:0.01), (MELPERONE:Breast cancer:0.23), (MENTHOL\ZINC OXIDE:Breast cancer:0.07), (MEPOLIZUMAB:Breast cancer:0.83), (METAXALONE:Breast cancer:2.5), (METFORMIN HYDROCHLORIDE\SAXAGLIPTIN HYDROCHLORIDE:Breast cancer:0.54), (METHENAMINE MANDELATE:Breast cancer:0.11), (METHOXY POLYETHYLENE GLYCOL-EPOETIN BETA:Breast cancer:0.16), (METRELEPTIN:Breast cancer:0.18), (MEXILETINE:Breast cancer:0.14), (MICAFUNGIN:Breast cancer:0.38), (MICONAZOLE NITRATE:Breast cancer:0.45), (MIDODRINE HYDROCHLORIDE:Breast cancer:1), (MIGLUSTAT:Breast cancer:0.16), (MINERALS:Breast cancer:0.38), (MINODRONIC ACID:Breast cancer:0.18), (MITIGLINIDE:Breast cancer:0.01), (MIZORIBINE:Breast cancer:0.48), (MOLSIDOMINE:Breast cancer:0.18), (NALOXONE:Breast cancer:0.39), (NEOMYCIN:Breast cancer:0.07), (NIACINAMIDE:Breast cancer:0.01), (NISOLDIPINE:Breast cancer:0.2), (NOREPINEPHRINE BITARTRATE:Breast cancer:0.19), (OLOPATADINE:Breast cancer:0.34), (OSIMERTINIB:Breast cancer:1.13), (PASIREOTIDE:Breast cancer:0.9), (PATIROMER:Breast cancer:0.83), (PEGINTERFERON ALFA-2B:Breast cancer:0.3), (PENICILLIN G:Breast cancer:0.99), (PENTAMIDINE ISETHIONATE:Breast cancer:0.32), (PENTOSTATIN:Breast cancer:0.94), (PILSICAINIDE HYDROCHLORIDE:Breast cancer:0.75), (PIMOBENDAN:Breast cancer:0.01), (PIRITRAMIDE:Breast cancer:0.25), (PITOFENONE:Breast cancer:0.06), (PORFIMER SODIUM:Breast cancer:0.07), (POTASSIUM CITRATE\TRISODIUM CITRATE DIHYDRATE:Breast cancer:0.07), (PREDNISOLONE SODIUM PHOSPHATE:Breast cancer:0.16), (PROCATEROL HYDROCHLORIDE:Breast cancer:0.04), (PROTRIPTYLINE HYDROCHLORIDE:Breast cancer:0.5), (PRUCALOPRIDE SUCCINATE:Breast cancer:0.11), (PYRAZINAMIDE:Breast cancer:0.79), (QUINACRINE:Breast cancer:0.05), (RASBURICASE:Breast cancer:0.58), (RILMENIDINE PHOSPHATE:Breast cancer:0.49), (RILPIVIRINE HYDROCHLORIDE:Breast cancer:0.09), (ROFECOXIB:Breast cancer:0.38), (SAMARIUM SM-153 LEXIDRONAM PENTASODIUM:Breast cancer:0.48), (SAW PALMETTO:Breast cancer:0.79), (SELEGILINE HYDROCHLORIDE:Breast cancer:0.36), (SELEXIPAG:Breast cancer:0.63), (SILICON DIOXIDE:Breast cancer:0.06), (SILODOSIN:Breast cancer:1.23), (SIPULEUCEL-T:Breast cancer:0.9), (SODIUM FLUORIDE:Breast cancer:1.3), (SORBITOL:Breast cancer:0.19), (SUFENTANIL CITRATE:Breast cancer:0.04), (SULINDAC:Breast cancer:0.76), (SUPLATAST TOSILATE:Breast cancer:0.38), (TEDUGLUTIDE\WATER:Breast cancer:0.36), (TELOTRISTAT ETHYL:Breast cancer:0.81), (TERAZOSIN HYDROCHLORIDE:Breast cancer:1.42), (TESTOSTERONE CYPIONATE:Breast cancer:0.41), (TESTOSTERONE UNDECANOATE:Breast cancer:1.19), (THEOPHYLLINE:Breast cancer:0.61), (THIOCTIC ACID:Breast cancer:0.33), (TIBOLONE:Breast cancer:0.39), (TILIDINE:Breast cancer:0.49), (TIMOLOL MALEATE\TRAVOPROST:Breast cancer:0.35), (TIMOLOL\TRAVOPROST:Breast cancer:0), (TRIFLURIDINE:Breast cancer:0.05), (TRIHEXYPHENIDYL:Breast cancer:0.39), (VALGANCICLOVIR HYDROCHLORIDE:Breast cancer:1.37), (VANDETANIB:Breast cancer:0.65), (ZAFIRLUKAST:Breast cancer:1.11), (ZICONOTIDE ACETATE:Breast cancer:0.11), (ZILEUTON:Breast cancer:0.64), (ABIRATERONE:Breast cancer female:0.01), (ABIRATERONE ACETATE:Breast cancer female:0.84), (ACARBOSE:Breast cancer female:0.34), (AFATINIB:Breast cancer female:0.47), (ALBIGLUTIDE:Breast cancer female:0.16), (ALCOHOL:Breast cancer female:0.46), (AMILORIDE HYDROCHLORIDE\HYDROCHLOROTHIAZIDE:Breast cancer female:0.1), (AMISULPRIDE:Breast cancer female:0.28), (AMLODIPINE BESYLATE\HYDROCHLOROTHIAZIDE\VALSARTAN:Breast cancer female:0.19), (AMLODIPINE\VALSARTAN:Breast cancer female:0.36), (ANTIHISTAMINES NOS:Breast cancer female:0.2), (AXITINIB:Breast cancer female:0.7), (AZELASTINE:Breast cancer female:0.54), (BACITRACIN:Breast cancer female:0.59), (BUPRENORPHINE HYDROCHLORIDE:Breast cancer female:0.4), (BUPRENORPHINE HYDROCHLORIDE\NALOXONE HYDROCHLORIDE:Breast cancer female:0.33), (CALCITONIN SALMON:Breast cancer female:1.01), (CANAGLIFLOZIN:Breast cancer female:0.57), (CANAGLIFLOZIN\METFORMIN HYDROCHLORIDE:Breast cancer female:0.39), (CANAKINUMAB:Breast cancer female:0.62), (CARFILZOMIB:Breast cancer female:1.12), (CERITINIB:Breast cancer female:0.3), (CERTOLIZUMAB PEGOL:Breast cancer female:0.87), (CETUXIMAB:Breast cancer female:0.66), (CEVIMELINE HYDROCHLORIDE:Breast cancer female:0.1), (CHONDROITIN SULFATE A\GLUCOSAMINE:Breast cancer female:0.04), (CILASTATIN SODIUM\IMIPENEM:Breast cancer female:0.11), (CLINDAMYCIN PHOSPHATE:Breast cancer female:0.59), (CLOZAPINE:Breast cancer female:0.96), (CORTICOTROPIN:Breast cancer female:0.19), (DABIGATRAN:Breast cancer female:0.07), (DABRAFENIB:Breast cancer female:0.33), (DABRAFENIB MESYLATE:Breast cancer female:0.2), (DACLIZUMAB:Breast cancer female:0.11), (DALTEPARIN:Breast cancer female:0.42), (DARATUMUMAB:Breast cancer female:0), (DEFLAZACORT:Breast cancer female:0.29), (DESONIDE:Breast cancer female:0.15), (DEVICE:Breast cancer female:0.56), (DEXAMETHASONE ACETATE:Breast cancer female:0.09), (DIGITOXIN:Breast cancer female:0.13), (DIMENHYDRINATE:Breast cancer female:0.39), (DIPYRONE:Breast cancer female:0.32), (DRONEDARONE:Breast cancer female:0.2), (DUTASTERIDE:Breast cancer female:1.37), (EDOXABAN TOSYLATE:Breast cancer female:0.91), (EMPAGLIFLOZIN\LINAGLIPTIN:Breast cancer female:0.45), (ESOMEPRAZOLE MAGNESIUM\NAPROXEN:Breast cancer female:0.43), (ESTROGENS, CONJUGATED\MEDROXYPROGESTERONE ACETATE:Breast cancer female:0.5), (ESTRONE\SUS SCROFA OVARY:Breast cancer female:0.07), (EVOLOCUMAB:Breast cancer female:0.87), (FLUVASTATIN SODIUM:Breast cancer female:0.11), (FLUVOXAMINE MALEATE:Breast cancer female:0.03), (FORMOTEROL FUMARATE:Breast cancer female:0.69), (FORMOTEROL FUMARATE\MOMETASONE FUROATE:Breast cancer female:0.02), (GLYCOPYRROLATE:Breast cancer female:0.05), (GLYCOPYRRONIUM\INDACATEROL:Breast cancer female:0.47), (HYALURONATE SODIUM:Breast cancer female:0.22), (HYDROCHLOROTHIAZIDE\LOSARTAN:Breast cancer female:0.13), (HYDROXYCHLOROQUINE DIPHOSPHATE:Breast cancer female:0.19), (ILOPROST:Breast cancer female:0.88), (INDAPAMIDE\PERINDOPRIL:Breast cancer female:0.58), (INTERFERON ALFA-2B:Breast cancer female:0.28), (IXAZOMIB:Breast cancer female:0.32), (KETOROLAC\KETOROLAC TROMETHAMINE:Breast cancer female:0.61), (LACOSAMIDE:Breast cancer female:0.25), (LEDIPASVIR\SOFOSBUVIR:Breast cancer female:0.65), (LERCANIDIPINE:Breast cancer female:0.49), (LEVOLEUCOVORIN CALCIUM:Breast cancer female:0.02), (LOMITAPIDE MESYLATE:Breast cancer female:0.51), (MEDROXYPROGESTERONE ACETATE:Breast cancer female:0.02), (MEPERIDINE:Breast cancer female:0.01), (MIDODRINE:Breast cancer female:0.43), (MIDODRINE HYDROCHLORIDE:Breast cancer female:0.06), (MUPIROCIN\MUPIROCIN CALCIUM:Breast cancer female:0.45), (NATEGLINIDE:Breast cancer female:0.59), (NICOTINE:Breast cancer female:0.18), (NILOTINIB:Breast cancer female:1.6), (OLMESARTAN:Breast cancer female:0.21), (ORPHENADRINE:Breast cancer female:0), (OXYMETAZOLINE:Breast cancer female:0.06), (PANITUMUMAB:Breast cancer female:0.08), (PANTOPRAZOLE MAGNESIUM:Breast cancer female:1.48), (PATIROMER:Breast cancer female:0.26), (PEGINTERFERON BETA-1A:Breast cancer female:0.3), (PEMETREXED:Breast cancer female:0.73), (PENTOSAN POLYSULFATE SODIUM:Breast cancer female:0.14), (PHENYTOIN SODIUM:Breast cancer female:0.25), (PIRFENIDONE:Breast cancer female:0.21), (PRAZOSIN:Breast cancer female:0.23), (PROGESTERONE:Breast cancer female:0.87), (RADIUM RA-223 DICHLORIDE:Breast cancer female:0.93), (RAMUCIRUMAB:Breast cancer female:0.63), (SALMETEROL XINAFOATE:Breast cancer female:0.25), (SECUKINUMAB:Breast cancer female:0.4), (SILODOSIN:Breast cancer female:0.19), (SIPULEUCEL-T:Breast cancer female:0.02), (SODIUM OXYBATE:Breast cancer female:1.27), (SUMATRIPTAN\SUMATRIPTAN SUCCINATE:Breast cancer female:1.22), (TERAZOSIN HYDROCHLORIDE:Breast cancer female:0.32), (TERAZOSIN\TERAZOSIN HYDROCHLORIDE:Breast cancer female:0.1), (TERBUTALINE SULFATE:Breast cancer female:0.52), (TERIFLUNOMIDE:Breast cancer female:0.03), (THALIDOMIDE:Breast cancer female:1.3), (TIOTROPIUM:Breast cancer female:0.65), (TIPIRACIL HYDROCHLORIDE\TRIFLURIDINE:Breast cancer female:0.16), (TOCILIZUMAB:Breast cancer female:0.03), (TOFACITINIB CITRATE:Breast cancer female:0.21), (TRAMETINIB:Breast cancer female:0.25), (TRANDOLAPRIL:Breast cancer female:0.12), (VANDETANIB:Breast cancer female:0.1), (ZAFIRLUKAST:Breast cancer female:0.31), (ZILEUTON:Breast cancer female:0.18), (ZIV-AFLIBERCEPT:Breast cancer female:0.08), (ACETAMINOPHEN\CAFFEINE\OPIUM:Breast cancer metastatic:0.29), (ACETAMINOPHEN\OXYCODONE\OXYCODONE HYDROCHLORIDE:Breast cancer metastatic:1.11), (ACETAMINOPHEN\PROPOXYPHENE NAPSYLATE:Breast cancer metastatic:0.78), (ACETAZOLAMIDE:Breast cancer metastatic:1.84), (AGALSIDASE BETA:Breast cancer metastatic:0.37), (ALCOHOL:Breast cancer metastatic:0.68), (ALECTINIB:Breast cancer metastatic:0.03), (ALFUZOSIN:Breast cancer metastatic:0.36), (ALFUZOSIN HYDROCHLORIDE:Breast cancer metastatic:0.45), (AMLODIPINE BESYLATE\HYDROCHLOROTHIAZIDE\VALSARTAN:Breast cancer metastatic:0.3), (AMLODIPINE\PERINDOPRIL:Breast cancer metastatic:0.05), (AMPICILLIN SODIUM:Breast cancer metastatic:0.27), (ANTIHISTAMINES NOS:Breast cancer metastatic:0.4),(ARMODAFINIL:Breast cancer metastatic:1.39), (ATROPINE SULFATE:Breast cancer metastatic:0.16), (AZACITIDINE:Breast cancer metastatic:0.4), (BETAMETHASONE\CALCIPOTRIENE:Breast cancer metastatic:0.19), (BLINATUMOMAB:Breast cancer metastatic:0.13), (BUPRENORPHINE HYDROCHLORIDE\NALOXONE HYDROCHLORIDE:Breast cancer metastatic:0.39), (CANAGLIFLOZIN\METFORMIN HYDROCHLORIDE:Breast cancer metastatic:0.6), (CANAKINUMAB:Breast cancer metastatic:0.73), (CARFILZOMIB:Breast cancer metastatic:1.7), (CEFPODOXIME PROXETIL:Breast cancer metastatic:0.06), (CERITINIB:Breast cancer metastatic:1.19), (CERTOLIZUMAB PEGOL:Breast cancer metastatic:1.28), (CETUXIMAB:Breast cancer metastatic:1.22), (CHLORPHENIRAMINE\HYDROCODONE:Breast cancer metastatic:0.37), (CHLORPROMAZINE:Breast cancer metastatic:0.08), (CORTICOTROPIN:Breast cancer metastatic:0.22), (CYPROHEPTADINE:Breast cancer metastatic:0.55), (DABRAFENIB:Breast cancer metastatic:1.36), (DACLIZUMAB:Breast cancer metastatic:0.35), (DAPAGLIFLOZIN PROPANEDIOL:Breast cancer metastatic:0.24), (DASATINIB:Breast cancer metastatic:2.41), (DEGARELIX:Breast cancer metastatic:0.05), (DESONIDE:Breast cancer metastatic:0.43), (DEVICE:Breast cancer metastatic:0.25), (DIFLUNISAL:Breast cancer metastatic:0.05), (DORZOLAMIDE:Breast cancer metastatic:0.51), (DOXYLAMINE SUCCINATE:Breast cancer metastatic:0.03), (DRONEDARONE:Breast cancer metastatic:0.44), (DUTASTERIDE:Breast cancer metastatic:1.96), (ECONAZOLE:Breast cancer metastatic:0.75), (EDOXABAN TOSILATE MONOHYDRATE:Breast cancer metastatic:0.05), (EDOXABAN TOSYLATE:Breast cancer metastatic:1.29), (ELOTUZUMAB:Breast cancer metastatic:0.74), (EMPAGLIFLOZIN\LINAGLIPTIN:Breast cancer metastatic:0.29), (EPLERENONE:Breast cancer metastatic:0.77), (ERTAPENEM SODIUM:Breast cancer metastatic:0.05), (ESTROGENS:Breast cancer metastatic:0.48), (ESTRONE\SUS SCROFA OVARY:Breast cancer metastatic:0.73), (ETRETINATE:Breast cancer metastatic:0.08), (EVOLOCUMAB:Breast cancer metastatic:0.83), (FERROUS SULFATE\GLYCINE:Breast cancer metastatic:0.05), (FEXOFENADINE\PSEUDOEPHEDRINE:Breast cancer metastatic:0.32), (FLUDROCORTISONE:Breast cancer metastatic:0.24), (FLUDROCORTISONE ACETATE:Breast cancer metastatic:0.88), (FLUOROURACIL\IRINOTECAN\LEUCOVORIN:Breast cancer metastatic:0.46), (FLUOROURACIL\LEUCOVORIN CALCIUM\OXALIPLATIN:Breast cancer metastatic:0.66), (FLUTAMIDE:Breast cancer metastatic:0.42), (FLUTICASONE FUROATE:Breast cancer metastatic:0.61), (FORMOTEROL FUMARATE\MOMETASONE FUROATE:Breast cancer metastatic:0.1), (FUSIDIC ACID:Breast cancer metastatic:1.1), (HERBALS\SAW PALMETTO:Breast cancer metastatic:0.28), (HYDROCHLOROTHIAZIDE\QUINAPRIL HYDROCHLORIDE:Breast cancer metastatic:0.07), (HYDROCODONE BITARTRATE\IBUPROFEN:Breast cancer metastatic:0.35), (HYDROCORTISONE ACETATE:Breast cancer metastatic:0.33), (HYDROXYCHLOROQUINE DIPHOSPHATE:Breast cancer metastatic:0.63), (IDELALISIB:Breast cancer metastatic:0.55), (ILOPROST:Breast cancer metastatic:0.19), (IMIPRAMINE:Breast cancer metastatic:0.07), (INDACATEROL:Breast cancer metastatic:0.27), (INTERFERON:Breast cancer metastatic:0.28), (INTERFERON ALFA-2A:Breast cancer metastatic:0.42), (INTERFERON ALFA-2B:Breast cancer metastatic:1.31), (IRON\POLYSACCHARIDES:Breast cancer metastatic:0.19), (LABETALOL HYDROCHLORIDE:Breast cancer metastatic:0.68), (LANREOTIDE:Breast cancer metastatic:0.22), (LEDIPASVIR\SOFOSBUVIR:Breast cancer metastatic:0.48), (LEUCOVORIN CALCIUM:Breast cancer metastatic:1.07), (LISDEXAMFETAMINE DIMESYLATE:Breast cancer metastatic:0.67), (LOMITAPIDE MESYLATE:Breast cancer metastatic:0.18), (MESNA:Breast cancer metastatic:0.18), (METFORMIN HYDROCHLORIDE\VILDAGLIPTIN:Breast cancer metastatic:0.85), (METHENAMINE HIPPURATE:Breast cancer metastatic:1.14), (METOLAZONE:Breast cancer metastatic:0.27), (MIDODRINE HYDROCHLORIDE:Breast cancer metastatic:0.15), (NALOXONE:Breast cancer metastatic:0.04), (NATEGLINIDE:Breast cancer metastatic:0.53), (NIRAPARIB:Breast cancer metastatic:0.78), (NOREPINEPHRINE:Breast cancer metastatic:0.02), (NORETHINDRONE:Breast cancer metastatic:0.53), (OSIMERTINIB:Breast cancer metastatic:0.66), (PASIREOTIDE:Breast cancer metastatic:0.07), (PATIROMER:Breast cancer metastatic:0.04), (PEGINTERFERON BETA-1A:Breast cancer metastatic:0.38), (PENICILLIN V:Breast cancer metastatic:0.23), (PENTOSAN POLYSULFATE SODIUM:Breast cancer metastatic:0.28), (PENTOSTATIN:Breast cancer metastatic:0.12), (PHENYTOIN SODIUM:Breast cancer metastatic:0.93), (PIMECROLIMUS:Breast cancer metastatic:0.19), (PIPERACILLIN:Breast cancer metastatic:0.02), (PIRFENIDONE:Breast cancer metastatic:0.16), (PNEUMOCOCCAL VACCINE, POLYVALENT 23:Breast cancer metastatic:0.09), (PRAZOSIN:Breast cancer metastatic:0.27), (PRIMIDONE:Breast cancer metastatic:1.57), (PYRIDOSTIGMINE BROMIDE:Breast cancer metastatic:0.11), (RADIUM RA-223 DICHLORIDE:Breast cancer metastatic:1.13), (RAMUCIRUMAB:Breast cancer metastatic:1.5), (RANOLAZINE:Breast cancer metastatic:1.2), (REGORAFENIB:Breast cancer metastatic:1.92), (RETINOL:Breast cancer metastatic:0.73), (RIVASTIGMINE:Breast cancer metastatic:0.63), (RIVASTIGMINE TARTRATE:Breast cancer metastatic:0.72), (ROFLUMILAST:Breast cancer metastatic:0.8), (RUCAPARIB CAMSYLATE:Breast cancer metastatic:0.11), (SALMETEROL XINAFOATE:Breast cancer metastatic:0.03), (SECUKINUMAB:Breast cancer metastatic:0.28), (SEVELAMER CARBONATE:Breast cancer metastatic:0), (SILODOSIN:Breast cancer metastatic:0.52), (SIPULEUCEL-T:Breast cancer metastatic:0.39), (SODIUM OXYBATE:Breast cancer metastatic:0.91), (STARCH, WHEAT:Breast cancer metastatic:0.12), (SULINDAC:Breast cancer metastatic:0.2), (TAFLUPROST:Breast cancer metastatic:0.03), (TAZOBACTAM:Breast cancer metastatic:0.32), (TEDUGLUTIDE\WATER:Breast cancer metastatic:0.15), (TERAZOSIN HYDROCHLORIDE:Breast cancer metastatic:0.56), (TERAZOSIN\TERAZOSIN HYDROCHLORIDE:Breast cancer metastatic:0.15), (TERBUTALINE:Breast cancer metastatic:0.43), (THALIDOMIDE:Breast cancer metastatic:1.93), (TIPIRACIL HYDROCHLORIDE\TRIFLURIDINE:Breast cancer metastatic:0.55), (TOCILIZUMAB:Breast cancer metastatic:0.26), (TOLTERODINE:Breast cancer metastatic:0.19), (TRIMIPRAMINE MALEATE:Breast cancer metastatic:0.24), (VANCOMYCIN HYDROCHLORIDE:Breast cancer metastatic:1.06), (VINCRISTINE SULFATE:Breast cancer metastatic:0.44), (ZIV-AFLIBERCEPT:Breast cancer metastatic:0.78), (ZONISAMIDE:Breast cancer metastatic:0.48), (RISEDRONATE SODIUM:Breast cancer recurrent:0.01), (ALLOPURINOL:Breast cancer stage IV:0.08), (BICALUTAMIDE:Breast cancer stage IV:0.56), (BISOPROLOL FUMARATE:Breast cancer stage IV:0.12), (BUMETANIDE:Breast cancer stage IV:0.07), (CERTOLIZUMAB PEGOL:Breast cancer stage IV:0.06), (CLINDAMYCIN\CLINDAMYCIN PHOSPHATE:Breast cancer stage IV:0.17), (DEXLANSOPRAZOLE:Breast cancer stage IV:0.86), (EVOLOCUMAB:Breast cancer stage IV:0.29), (FERROUS SULFATE\FERROUS SULFATE, DRIED:Breast cancer stage IV:0.26), (FOLIC ACID:Breast cancer stage IV:1.3), (GLIPIZIDE:Breast cancer stage IV:0.09), (IBRUTINIB:Breast cancer stage IV:0.75), (NITROFURANTOIN:Breast cancer stage IV:0.21), (OXYGEN:Breast cancer stage IV:0.36), (PRAVASTATIN:Breast cancer stage IV:0.22), (RAMIPRIL:Breast cancer stage IV:0.58), (RISEDRONATE SODIUM:Breast cancer stage IV:0.22), (TAMSULOSIN:Breast cancer stage IV:0.25), (ACENOCOUMAROL:Bronchial carcinoma:0.2), (ADO-TRASTUZUMAB EMTANSINE:Bronchial carcinoma:0.01), (CALCITRIOL:Bronchial carcinoma:0.1), (CANDESARTAN CILEXETIL\HYDROCHLOROTHIAZIDE:Bronchial carcinoma:0.66), (CERTOLIZUMAB PEGOL:Bronchial carcinoma:0.29), (CLOTRIMAZOLE:Bronchial carcinoma:0.08), (COLCHICINE:Bronchial carcinoma:0.25), (DEXLANSOPRAZOLE:Bronchial carcinoma:0.79), (DICLOFENAC:Bronchial carcinoma:0.17), (EPIRUBICIN:Bronchial carcinoma:0.15), (EVOLOCUMAB:Bronchial carcinoma:0.22), (FERROUS SULFATE\FERROUS SULFATE, DRIED:Bronchial carcinoma:0.71), (FLECAINIDE:Bronchial carcinoma:0.23), (IBRUTINIB:Bronchial carcinoma:1.22), (INDAPAMIDE\PERINDOPRIL:Bronchial carcinoma:0.4), (INSULIN HUMAN:Bronchial carcinoma:0.3), (IRON:Bronchial carcinoma:0.31), (ISOSORBIDE MONONITRATE:Bronchial carcinoma:0.04), (LERCANIDIPINE HYDROCHLORIDE:Bronchial carcinoma:0.34), (MELATONIN:Bronchial carcinoma:0), (NILOTINIB:Bronchial carcinoma:0.28), (NITROGLYCERIN:Bronchial carcinoma:0.23), (OXYGEN:Bronchial carcinoma:0.4), (PANTOPRAZOLE MAGNESIUM:Bronchial carcinoma:0.11), (RANOLAZINE:Bronchial carcinoma:0.05), (SACUBITRIL\VALSARTAN:Bronchial carcinoma:0.21), (SPIRONOLACTONE:Bronchial carcinoma:1.27), (THALIDOMIDE:Bronchial carcinoma:0.18), (TOFACITINIB CITRATE:Bronchial carcinoma:0.22), (TOPIRAMATE:Bronchial carcinoma:0.42), (UMECLIDINIUM BROMIDE\VILANTEROL TRIFENATATE:Bronchial carcinoma:0.09), (CERTOLIZUMAB PEGOL:Bronchial disorder:0.04), (DIGOXIN:Bronchial disorder:0.08), (DILTIAZEM HYDROCHLORIDE:Bronchial disorder:0.29), (MEPOLIZUMAB:Bronchial disorder:0.36), (NAPROXEN:Bronchial disorder:0.29), (NEBIVOLOL:Bronchial disorder:0), (PANTOPRAZOLE:Bronchial disorder:0.26), (PANTOPRAZOLE MAGNESIUM:Bronchial disorder:0.16), (PYRAZINAMIDE:Bronchial disorder:0.59), (TAMSULOSIN:Bronchial disorder:0), (.ALPHA.1-PROTEINASE INHIBITOR HUMAN:Bronchial hyperreactivity:0.03), (AMITRIPTYLINE:Bronchial hyperreactivity:0.14), (BISOPROLOL FUMARATE:Bronchial hyperreactivity:0.05), (BUMETANIDE:Bronchial hyperreactivity:0.49), (CEPHALEXIN:Bronchial hyperreactivity:0.34), (CLINDAMYCIN\CLINDAMYCIN PHOSPHATE:Bronchial hyperreactivity:0.35), (CYCLOBENZAPRINE:Bronchial hyperreactivity:0.01), (DIPHENHYDRAMINE:Bronchial hyperreactivity:0.32), (ELETRIPTAN HYDROBROMIDE:Bronchial hyperreactivity:0.1), (ENALAPRIL:Bronchial hyperreactivity:0.51), (ENOXAPARIN:Bronchial hyperreactivity:0.26), (ENOXAPARIN SODIUM:Bronchial hyperreactivity:0.15), (EVOLOCUMAB:Bronchial hyperreactivity:0.27), (IBRUTINIB:Bronchial hyperreactivity:1.92), (ILOPROST:Bronchial hyperreactivity:0.43), (LOVASTATIN:Bronchial hyperreactivity:0.2), (MAGNESIUM:Bronchial hyperreactivity:0.17), (METRONIDAZOLE:Bronchial hyperreactivity:0.31), (NITROGLYCERIN:Bronchial hyperreactivity:0.02), (PRAVASTATIN:Bronchial hyperreactivity:0.2), (PRAVASTATIN SODIUM:Bronchial hyperreactivity:0.17), (VARENICLINE TARTRATE:Bronchial hyperreactivity:0.33), (AMIODARONE:Bronchiectasis:1.06), (AMIODARONE HYDROCHLORIDE:Bronchiectasis:0.64), (ANASTROZOLE:Bronchiectasis:0.57), (ARMODA FINIL:Bronchiectasis:0.14), (BENAZEPRIL HYDROCHLORIDE:Bronchiectasis:0.02), (BENAZEPRIL\BENAZEPRIL HYDROCHLORIDE:Bronchiectasis:0.02), (BORTEZOMIB:Bronchiectasis:0.37), (BRINZOLA MIDE:Bronchiectasis:0.12), (CALCIUM ACETATE:Bronchiectasis:0.22), (CANDESARTAN:Bronchiectasis:0.64), (CEFOTAXIME SODIUM:Bronchiectasis:0.37), (CHOLESTYRAMINE:Bronchiectasis:0.33), (CILASTATIN SODIUM\IMIPENEM:Bronchiectasis:0.4), (CYCLOBENZAPRINE:Bronchiectasis:0.46), (CYP ROHEPTADINE:Bronchiectasis:0.25), (DASATINIB:Bronchiectasis:0.66), (DEVICE:Bronchiectasis: 0.05), (DICYCLOMINE HYDROCHLORIDE:Bronchiectasis:0.13), (DIMENHYDRINATE:Bronchiectasis:0.15), (DOXA ZOSIN MESYLATE:Bronchiectasis:0.16), (DULOXETINE:Bronchiectasis:0.11), (ELETRIPTAN HYDROBROMIDE:Bronchiectasis:0.41), (EPLERENONE:Bronchiectasis:0.2), (ESZOPICLON E:Bronchiectasis:0.41), (EVEROLIMUS:Bronchiectasis:0.24), (EVOLOCUMAB:Bronchiectasis: 0.62), (FEBUXOSTAT:Bronchiectasis:0.02), (FESOTERODINE FUMARATE:Bronchiectasis:0.11), (FORMOTEROL FUMARATE\MOMETASONE FUROATE:Bronchiectasis:1.37), (GLICLAZIDE:Bronchiectasis:0.14), (GLYCOPYRRONIUM: Bronchiectasis:1.12), (HYDROCHLOROTHIAZIDE\IRBESARTAN:Bronchiectasis:0.2), (HYD ROCHLOROTHIAZIDE\LISINOPRIL:Bronchiectasis:0.6), (HYDROMORPHONE:Bronchiectasis: 0.54), (HYDROMORPHONE HYDROCHLORIDE:Bronchiectasis:0.07), (ILOPROST:Bronchiectasis:0.68), (IMIPRAMINE HYDROCHLORIDE:Bronchiectasis:0.03), (INDAPAMIDE\PERINDOPRIL:Bronchiectasis:0.07), (INDOMETHACIN:Bronchiectasis:0.1), (LEDIPASVIR\SOFOSBUVIR:Bronchiectasis:0.14), (LERCANIDIPINE:Bronchiectasis:0.18), (LITHIUM CARBONATE:Bronchiectasis:0.2), (METHADONE HYDROCHLORIDE:Bronchiectasis:0.09), (MICAFUNGIN:Bronchiectasis:0.2), (NEBIVOLOL HYDROCHLORIDE:Bronchiectasis:0.32), (NINTEDANIB:Bronchiectasis:0.31), (NORTRIPTY LINE:Bronchiectasis:0.07), (OLANZAPINE:Bronchiectasis:0.17), (OLOPATADINE:Bronchiectasis: 0.34), (OMEGA-3-ACID ETHYL ESTERS:Bronchiectasis:0.34), (OSELTAMIVIR:Bronchiectasis:0.01), (PANTOPRAZOLE MAGNESIUM:Bronchiectasis:1.74), (PAROXETINE:Bronchiectasis:0.2), (PENICILLIN:Bronchiectasis: 0.03), (PONATINIB:Bronchiectasis:0.15), (PRAVASTATIN SODIUM:Bronchiectasis:1.01), (QUETIAPINE:Bronchiectasis:0.03), (SACUBITRIL\VALSART AN:Bronchiectasis:0.47), (SECUKINUMAB:Bronchiectasis:0.53), (SELEXIPAG:Bronchiectasis: 0.37), (SODIUM OXYBATE:Bronchiectasis:0.24), (SULFAMETHOXAZOLE:Bronchiectasis:0.31), (THALIDO MIDE:Bronchiectasis:0.1), (VANCOMYCIN HYDROCHLORIDE:Bronchiectasis:0.36), (VITAMIN B:Bronchiectasis:0.31), (ZOPICLONE:Bronchiectasis:0.26), (ABOBOTULINUMTOXINA:Bronchitis: 0.32), (ACETAMINOPHEN\CAFFEINE:Bronchitis:0.51), (ACETAMINOPHEN\OXYCO DONE\OXYCODONE HYDROCHLORIDE:Bronchitis:1.67), (ACETAMINOPHEN\PROPOXYPHENE NAPSYLATE:Bronchitis:0.61), (ACITRETIN:Bronchitis:0.57), (ACTIVATED CHARCOAL:Bronchitis:0.38), (ADO-TRASTUZUMAB EMTANSINE:Bronchitis:1.07), (AGALSIDASE BETA:Bronchitis:1.28), (ALBUMIN HUMAN:Bronchitis:0.48), (ALBUTEROL\GUAIFENESIN:Bronchitis:0.38), (ALGLUCOSIDA SE ALFA:Bronchitis:1.51), (ALUMINUM HYDROXIDE\MAGNESIUM OXIDE\OXETHAZAINE:Bronchitis:0.24), (AMINO ACIDS\CARBOHYDRATES\MINERALS\SOYBEAN OIL\VITAMINS:Bronchitis:0.15), (AMLODIPINE BESYLATE\HYDROCHLOROTHIAZIDE\OLMESARTAN MEDOXOMIL:Bronchitis:0.75), (AMOXAPINE:Bronchitis:0.83), (AMOXICILLIN\CLARITHR OMYCIN\ESOMEPRAZOLE:Bronchitis:0.37), (ANTIHISTAMINES NOS:Bronchitis:0.88), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Bronchitis:0.04), (ASPIRIN\MAGNESIUM OXIDE:Bronchitis:0.59), (ASUNAPREVIR:Bronchitis:0.22), (ATROPINE SULFATE:Bronchitis:0.54), (AXITINIB:Bronchitis:1.04), (AZILSARTAN KAMEDOXOMIL:Bronchitis:2.06), (BELATACEPT:Bronchitis:0.5), (BENZTROPINE:Bronchit is: 0.21), (BETAMETHASONE BUTYRATE PROPIONATE:Bronchitis:0.23), (BETAMETHASONE DIPROPIONATE\CALCIPOTRIENE:Bronchitis:0.37), (BETAMETHASONE DIPROPIONATE\CALCIPOTRIENE HYDRATE:Bronchitis:0.02), (BETAMETHASONE DIPROPIONATE\CLOTRIMAZOLE:Bronchitis:0.61), (BETAMETHASONE\CALCIPOTRIE NE:Bronchitis:0.81), (BETHANECHOL CHLORIDE:Bronchitis:0.51), (BEZAFIBRATE:Bronchitis:0.13), (BILASTINE:Bronchitis:1.06), (BLINATUMOMAB:Bronchitis:0.31), (BOTULINUM TOXIN TYPE A:Bronchitis:0.34), (BRINZOLAMIDE\TIMOLOL MALEATE:Bronchitis:0.07), (BUTALBITAL:Bronchitis:0.12), (CABOZANTINIB S-MALATE:Bronchitis:0.11), (CALCIPOTRIENE:Bronchitis:0.91), (CALCITONIN SALMON:Bronchitis:1.27), (CALCIUM CARBONATE\ERGOCALCIFEROL\RETINOL:Bronchitis:0.71), (CALCIUM CHLORIDE\DEXTROSE\POTASSIUM CHLORIDE\SODIUM ACETATE\SODIUM CHLORIDE:Bronchitis:0.18), (CALCIUM\MAGNESIUM\ZINC:Bronchitis:0.45), (CAMPHOR\MENTHOL\METHYL SALICYLATE:Bronchitis:0.53), (CANAGLIFLOZIN\METFORMIN HYDROCHLORIDE:Bronchitis:1.28), (CARVEDILOL PHOSPHATE:Bronchitis:2.19), (CASANTHRANOL\DOCUSATE SODIUM:Bronchitis:0.54), (CASPOFUNGIN:Bronchitis:0.5), (CEFOTAXIME\CEFOTAXIME SODIUM:Bronchitis:0.74), (CEPHARANTHINE:Bronchitis:0.24), (CETUXIMAB:Bronchitis:0.99), (CEVIMELINE HYDROCHLORIDE:Bronchitis:0.43), (COLISTIN:Bronchitis:0.54), (CORTICOTROPIN:Bronchitis: 1.09), (DABIGATRAN:Bronchitis:1.03), (DANAZOL:Bronchitis:0.88), (DANTROLENE SODIUM:Bronchitis:0.43), (DEGARELIX:Bronchitis:0.41), (DEXAMETHASONE ACETATE:Bronchitis:2.09), (DIMETHICONE\SILICON DIOXIDE\SIMETHICONE:Bronchitis:0.5), (DIPHENIDOL HYDROCHLORIDE:Bronchitis:0.59), (DIPYRONE\PROMETHAZINE HYDROCHLORIDE:Bronchitis:0.03), (DISTIGMINE BROMIDE:Bronchitis:0.2), (DOLASETRON MESYLATE:Bronchitis:0.3), (DOXEPIN HYDROCHLORIDE:Bronchitis:1.1), (DRONABINOL:Bronchitis:1.54), (ELBASVIR\GRAZOP REVIR:Bronchitis:0.16), (ELTROMBOPAG OLAMINE:Bronchitis:0.41), (EMPAGLIFLOZIN\LINAGLIPTIN:Bronchitis:0.96), (EPERISON E HYDROCHLORIDE:Bronchitis:0.73), (EPHEDRINE HYDROCHLORIDE\GUAIFENESIN:Bronchitis:0.03), (EPIRUBICIN:Bronchitis:0.69), (ESOM EPRAZOLE MAGNESIUM\NAPROXEN:Bronchitis:0.36), (ESTROGENS:Bronchitis:0.34), (ETHACRYNIC ACID:Bronchitis:0.43), (ETHINYL ESTRADIOL\NORETHINDRONE:Bronchitis:0.18), (ETHINYL ESTRADIOL\NORGESTREL:Bronchitis:0.3), (ETRETINATE:Bronchitis:1.6), (FAROPENEM SODIUM:Bronchitis:0.42), (FELBAMATE:Bronchitis:0.13), (FLUDROCORTISONE ACETATE:Bronchitis:1.56), (FLUOCINOLONE ACETONIDE:Bronchitis:1.08), (FLUTICASONE PROPIONATE\FORMOTEROL FUMARATE:Bronchitis:0.73), (FULVESTRANT:Bronchitis:0.36), (GIMERACIL\OTERACIL\TEGAFUR:Bronchitis:0.75), (GINGER:Bronchitis:0.06), (GLYCERIN:Bronchitis:0.77), (GLYCE RIN\HYPROMELLOSES\POLYETHYLENE GLYCOL 400:Bronchitis:0.02), (GUANABENZ ACETATE:Bronchitis:0.06), (GUANFACINE:Bronchitis:0.57), (GUANFACINE HYDROCHLORIDE:Bronchitis:0.16), (HYDROCHLOROTHIAZIDE\RAMIPRIL:Bronchitis:0.18), (HYDROCHLOROTHIAZIDE\SPIRONOLACTONE:Bronchitis:0.15), (HYDROCODONE BITARTRATE\IBUPROFEN:Bronchitis:0.66), (HYDROXYCHLOROQUINE DIPHOSPHATE:Bronchitis:0.76), (HYOSCYAMINE SULFATE:Bronchitis:0.57), (IBRITUMOMAB TIUXETAN:Bronchitis:0.21), (IGURATIMOD:Bronchitis:0.35), (ILOPROST:Bronchitis:1.03), (I MATINIB MESYLATE:Bronchitis:1.84), (IMIGLUCERASE:Bronchitis:0.62), (IMIPRAMINE:Bronchitis:00.18), (INDAPAMIDE\PERINDOPRIL:Bronchitis:2.87), (INOTUZUMAB OZOGAMICIN:Bronchitis:0.3), (INTERFERON ALFA-2A:Bronchitis:1), (INTERFERON ALFA-2B:Bronchitis:0.46), (IRINOTECAN HYDROCHLORIDE:Bronchitis:0.86), (ISONIAZID\PYRAZINAMIDE\RIFAMPIN:Bronchitis: 0.28), (KETOTIFEN:Bronchitis:0.23), (LABETALOL HYDROCHLORIDE:Bronchitis:2.04), (LACTOBACILLUS RHAMNOSUS:Bronchitis:1.3), (LANREOTIDE:Bronchitis:0.05), (LAPATINIB DITOSYLATE:Bronchitis:0.38), (LENVATINIB:Bronchitis:0.12), (LEVOMILNACIPRAN HYDROCHLORIDE:Bronchitis:0.09), (LEVOTHYROXINE SODIUM\POTASSIUM IODIDE:Bronchitis:0.45), (LOXOPROFEN SODIUM DIHYDRATE:Bronchitis:0.12), (LUMACAFTOR:Bronchitis:0.76), (LURASIDONE HYDROCHLORIDE:Bronchitis:0.31), (LUTETIUM LU-177:Bronchitis:0.01), (MAGNESIUM ASPARTATE HYDROCHLORIDE:Bronchitis:0.44), (MELPERONE:Bronchitis:0.29), (MEPOLIZUMAB:Bronchitis: 2.27), (MEXILETINE HYDROCHLORIDE:Bronchitis:0.44), (MIDODRINE HYDROCHLORIDE:Bronchitis:0.94), (MIGLITOL:Bronchitis:0.39), (MIGLUSTAT:Bronchitis: 0.83), (MOEXIPRIL HYDROCHLORIDE:Bronchitis:0.23), (MORPHINE HYDROCHLORIDE:Bronchitis:0.31), (MORPHINE SULFATE\NALTREXONE HYDROCHLORIDE:Bronchitis:0.37), (NALOXONE:Bronchitis:0.17), (NARATRIPTAN HYDROCHLORIDE:Bronchitis:0.19), (NIACINAMIDE:Bronchitis:0.04), (NIRAPARIB:Bronchitis: 0.44), (NOREPINEPHRINE:Bronchitis:0.41), (NOREPINEPHRINE BITARTRATE:Bronchitis:0.07), (NOREPINEPHRINE\NOREPINEPHRINE BITARTRATE:Bronchitis:0.06), (OCTREOTIDE:Bronchitis:0.27), (OCTREOTIDE ACETATE:Bronchitis:0.61), (OLMESARTAN:Bronchitis:0.73), (OXAPROZIN:Bronchitis:0.01), (OXYBUTYNIN\OXYBUTYNIN CHLORIDE:Bronchitis:0.29), (PALIPERIDONE PALMITATE:Bronchitis:0.16), (PANCRELIPASE\PANCRELIPASE AMYLASE\PANCRELIPASE LIPASE\PANCRELIPASE PROTEASE:Bronchitis:0.4), (PANITUMUMAB:Bronchitis:0.51), (PATIROMER:Bronchitis:0.62), (PENTOSTATIN:Bronchitis:0.03), (PERTUZUMAB:Bronchitis:0.09), (PILSICAINIDE HYDROCHLORIDE:Bronchitis:0.94), (PIMECROLIMUS:Bronchitis:0.38), (PIMOBENDAN:Bronchitis: 0.71), (PIRITRAMIDE:Bronchitis:0.23), (PNEUMOCOCCAL VACCINE, POLYVALENT 23:Bronchitis:0.91), (PORFIMER SODIUM:Bronchitis:0.35), (POTASSIUM CHLORIDE\SODIUM CHLORIDE\SODIUM LACTATE:Bronchitis:0.07), (POTASSIUM CITRATE\TRISODIUM CITRATE DIHYDRATE:Bronchitis:0.84), (PROTRIPTYLINE HYDROCHLORIDE:Bronchitis:0.37), (PYRIDOXINE:Bronchitis:0.63), (PYRIMETHAMINE:Bronchitis: 0.02), (QUINACRINE:Bronchitis:0.84), (REGORAFENIB:Bronchitis:1.11), (RILPIVIR INE HYDROCHLORIDE:Bronchitis:0.61), (RIZATRIPTAN:Bronchitis:0.63), (SARPOGRELATE HYDROCHLORIDE:Bronchitis:0.33), (SELEGILINE:Bronchitis:0.13), (SOLIFENACIN:Bronchitis: 0.02), (SUPLATAST TOSILATE:Bronchitis:0.66), (TEDUGLUTIDE\WATER:Bronchitis:0.35), (TELOTRISTAT ETHYL:Bronchitis:0.11), (TEMSIROLIMUS:Bronchitis:0.05), (TESTOSTERONE UNDECANOATE:Bronchitis:1.2), (TIMOLOL MALEATE\TRAVOPROST:Bronchitis:0.22), (TINZAPARIN:Bronchitis:0.92), (TIPIRACIL HYDROCHLORIDE\TRIFLURIDINE:Bronchitis:0.41), (TOCOPHEROL:Bronchitis:0.83), (TRIHEXYPHENIDYL: Bronchitis:0.35), (VINORELBINE TARTRATE:Bronchitis:0.5), (VONOPRAZAN FUMURATE:Bronchitis:0.28), (ACETAMINOPHEN\OXYCODONE HYDROCHLORIDE\OXYCODONE TEREPHTHALATE:Bronchitischronic: 0.13), (ACETAMINOPHEN\OXYCODONE\OXYCODONE HYDROCHLORIDE:Bronchitis chronic:0.43), (ALBIGLUTIDE:Bronchitis chronic:0.03), (ALENDRONIC ACID:Bronchitis chronic:0.07), (AMISULPRIDE:Bronchitis chronic:0.44), (AMLODIPINE\VALSARTAN:Bronchitis chronic:0.17), (BERAPROST:Bronchitis chronic:0), (CALCIUM CARBONATE\CHOLECALCIFEROL:Bronchitis chronic:1.5), (CANDESARTAN CILEXETIL\HYDROCHLOROTHIAZIDE:Bronchitis chronic:1.12), (CARVEDILOL PHOSPHATE:Bronchitis chronic:1.28), (CEFDITOREN PIVOXIL:Bronchitis chronic:0.21), (CEFOTAXIME SODIUM:Bronchitis chronic:0.1), (CHLORPHENIRAMINE\HYDROCODONE:Bronchitis chronic:0.1), (CHOLESTYRAMINE:Bronchitis chronic:1.07), (CILASTATIN SODIUM\IMIPENEM:Bronchitis chronic:0.27), (CIMETIDINE:Bronchitis chronic:0.52), (CORTICOTROPIN:Bronchitis chronic:0.05), (CYPROHEPTADINE:Bronchitis chronic:0.08), (DABIGATRAN:Bronchitis chronic:0.29), (DACLIZUMAB:Bronchitis chronic:0.13), (DALFAMPRIDINE:Bronchitis chronic:0.03), (DANAZOL:Bronchitis chronic:0.24), (DAPAGLIFLOZIN PROPANEDIOL:Bronchitis chronic:0.06), (DEFLAZACORT:Bronchitis chronic:0.42), (DIGITOXIN:Bronchitis chronic:0.02), (DILAZEP:Bronchitis chronic:0.02), (DOXEPIN HYDROCHLORIDE:Bronchitis chronic:0.23), (ENOXAPARIN:Bronchitis chronic:1.64), (EPERISONE HYDROCHLORIDE:Bronchitis chronic:0.02), (ESTROGENS, CONJUGATED\MEDROXYPROGESTERONE ACETATE:Bronchitis chronic:0.4), (EVOLOCUMAB:Bronchitis chronic:1.24), (FILGRASTIM:Bronchitis chronic:0.09), (FLAVIN ADENINE DINUCLEOTIDE:Bronchitis chronic:0.09), (FLUDROCORTISONE ACETATE:Bronchitis chronic:0.19), (FORMOTEROL FUMARATE\MOMETASONE FUROATE:Bronchitis chronic:1.29), (GEMFIBROZIL:Bronchitis chronic:0), (GUANFACINE:Bronchitis chronic:0.03), (HERBALS\TURMERIC:Bronchitis chronic:0.08), (HYDROCHLOROTHIAZIDE\TELMISARTAN:Bronchitis chronic:0.46), (HYDROXYUREA:Bronchitis chronic:0.56), (HYDROXYZINE PAMOATE:Bronchitis chronic:1.36), (HYOSCYAMINE:Bronchitis chronic:0.5), (ICOSAPENT ETHYL:Bronchitis chronic:0.19), (ILOPROST:Bronchitis chronic:1.62), (IMIPRAMINE HYDROCHLORIDE:Bronchitis chronic:0.38), (INDAPAMIDE\PERINDOPRIL:Bronchitis chronic:0.56), (KETOROLAC\KETOROLAC TROMETHAMINE:Bronchitis chronic:0.03), (LACTASE:Bronchitis chronic:0), (LEDIPASVIR\SOFOSBUVIR:Bronchitis chronic:1.07), (LERCANIDIPINE:Bronchitis chronic:0.66), (LERCANIDIPINE HYDROCHLORIDE:Bronchitis chronic:0.56), (LIDOCAINE\PRILOCAINE:Bronchitis chronic:0.27), (METHENAMINE HIPPURATE:Bronchitis chronic:0.39), (METHENOLONE ENANTHATE:Bronchitis chronic:0.06), (METHIMAZOLE:Bronchitis chronic:0.32), (METHYLPREDNISOLONE ACETATE:Bronchitis chronic:0.35), (MIDODRINE:Bronchitis chronic:0.14), (MINOCYCLINE\MINOCYCLINE HYDROCHLORIDE:Bronchitis chronic:0.66), (NALOXONE HYDROCHLORIDE\OXYCODONE HYDROCHLORIDE:Bronchitis chronic:0.76), (NATEGLINIDE:Bronchitis chronic:0.38), (NEVIRAPINE:Bronchitis chronic:0.1), (NITROFURANTOIN:Bronchitis chronic:1.08), (OLOPATADINE:Bronchitis chronic:0.1), (OMEPRAZOLE SODIUM:Bronchitis chronic:0.25), (OXAZEPAM:Bronchitis chronic:0.25), (PANTOPRAZOLE MAGNESIUM:Bronchitis chronic:2), (PAROXETINE\PAROXETINE HYDROCHLORIDE:Bronchitis chronic:0.85), (PAZOPANIB HYDROCHLORIDE:Bronchitis chronic:0.79), (PEGINTERFERON BETA-1A:Bronchitis chronic:0.15), (PEMETREXED DISODIUM:Bronchitis chronic:0.1), (PILSICAINIDE HYDROCHLORIDE:Bronchitis chronic:0.67), (PONATINIB:Bronchitis chronic:0.38), (PREDNISOLONE ACETATE:Bronchitis chronic:0.26), (PRIMIDONE:Bronchitis chronic:1.37), (PROPAFENONE HYDROCHLORIDE:Bronchitis chronic:0.42), (PYRIDOXINE:Bronchitis chronic:0.39), (PYRIDOXINE HYDROCHLORIDE:Bronchitis chronic:0.56), (RALOXIFENE HYDROCHLORIDE:Bronchitis chronic:0.75), (ROSUVASTATIN:Bronchitis chronic:0.44), (SCOPOLAMINE:Bronchitis chronic:0.72), (SUMATRIPTAN\SUMATRIPTAN SUCCINATE:Bronchitis chronic:0.91), (SUPLATAST TOSILATE:Bronchitis chronic:0.33), (TERIFLUNOMIDE:Bronchitis chronic:0.12), (TETRACYCLINE:Bronchitis chronic:0.11), (THALIDOMIDE:Bronchitis chronic:0.66), (TIMOLOL MALEATE:Bronchitis chronic:0.09), (TOCOPHEROL:Bronchitis chronic:0.07), (VANCOMYCIN HYDROCHLORIDE:Bronchitis chronic:0.11), (VILAZODONE HYDROCHLORIDE:Bronchitis chronic:0.21), (VOGLIBOSE:Bronchitis chronic:0.27), (WARFARIN POTASSIUM:Bronchitis chronic:0.5), (ZINC:Bronchitis chronic:0.13), (ZONISAMIDE:Bronchitis chronic:0), (PYRAZINAMIDE:Bronchopneumonia:0.57), (.ALPHA.1-PROTEINASE INHIBITOR HUMAN:Bronchopulmonary aspergillosis:0.32), (ACETAMINOPHEN\CODEINE PHOSPHATE:Bronchopulmonary aspergillosis:0.52), (ALBUTEROL SULFATE\IPRATROPIUM BROMIDE:Bronchopulmonary aspergillosis:0.6), (ANASTROZOLE:Bronchopulmonary aspergillosis:0.46), (BELATACEPT:Bronchopulmonary aspergillosis:0.2), (BISACODYL OR DOCUSATE SODIUM:Bronchopulmonary aspergillosis:0.02), (CALCITRIOL:Bronchopulmonary aspergillosis:0.42), (CALCIUM ACETATE:Bronchopulmonary aspergillosis:0.32), (CALCIUM\VITAMIN D:Bronchopulmonary aspergillosis:0.22), (CANAKINUMAB:Bronchopulmonary aspergillosis:0.36), (CANDESARTAN:Bronchopulmonary aspergillosis:0.94), (CANDESARTAN CILEXETIL\HYDROCHLOROTHIAZIDE:Bronchopulmonary aspergillosis:0.48), (CEFPIROME SULFATE:Bronchopulmonary aspergillosis:0.28), (CERTOLIZUMAB PEGOL:Bronchopulmonary aspergillosis:0.59), (CHOLESTYRAMINE:Bronchopulmonary aspergillosis:0.03), (CICLESONIDE:Bronchopulmonary aspergillosis:0.39), (CLOTRIMAZOLE:Bronchopulmonary aspergillosis:0.24), (DASATINIB:Bronchopulmonary aspergillosis:0.94), (DAUNORUBICIN HYDROCHLORIDE:Bronchopulmonary aspergillosis:0.29), (DEVICE:Bronchopulmonary aspergillosis:0.1), (DEXLANSOPRAZOLE:Bronchopulmonary aspergillosis:0.6), (DICLOFENAC:Bronchopulmonary aspergillosis:0.32), (DIPHENHYDRAMINE:Bronchopulmonary aspergillosis:0.93), (DIVALPROEX SODIUM:Bronchopulmonary aspergillosis:0.21), (DOXAZOSIN MESYLATE:Bronchopulmonary aspergillosis:0.18), (DUTASTERIDE:Bronchopulmonary aspergillosis:0.34), (ECONAZOLE:Bronchopulmonary aspergillosis:0.07), (ELOTUZUMAB:Bronchopulmonary aspergillosis:0.2), (ETOPOSIDE PHOSPHATE:Bronchopulmonary aspergillosis:0.19), (EVOLOCUMAB:Bronchopulmonary aspergillosis:0.5), (FENOTEROL HYDROBROMIDE\IPRATROPIUM BROMIDE:Bronchopulmonary aspergillosis:0.06), (FERROUS SULFATE:Bronchopulmonary aspergillosis:0.41), (FORMOTEROL FUMARATE\MOMETASONE FUROATE:Bronchopulmonary aspergillosis:0.4), (FOSFOMYCIN:Bronchopulmonary aspergillosis:0.68), (GEMTUZUMAB OZOGAMICIN:Bronchopulmonary aspergillosis:0.2), (GLICLAZIDE:Bronchopulmonary aspergillosis:0.19), (GUSPERIMUS TRIHYDROCHLORIDE:Bronchopulmonary aspergillosis:0.15), (HYDRALAZINE HYDROCHLORIDE:Bronchopulmonary aspergillosis:0.08), (HYDROCHLOROTHIAZIDE\IRBESARTAN:Bronchopulmonary aspergillosis:0), (HYDROCHLOROTHIAZIDE\LISINOPRIL:Bronchopulmonary aspergillosis:0.17), (IBRITUMOMAB TIUXETAN:Bronchopulmonary aspergillosis:1.49), (ILOPROST:Bronchopulmonary aspergillosis:0.31), (INDAPAMIDE\PERINDOPRIL:Bronchopulmonary aspergillosis:0.14), (INFLUENZA VIRUS VACCINE:Bronchopulmonary aspergillosis:0.28), (INOTUZUMAB OZOGAMICIN:Bronchopulmonary aspergillosis:0.08), (ISOSORBIDE MONONITRATE:Bronchopulmonary aspergillosis:0.29), (IVACAFTOR:Bronchopulmonary aspergillosis:0.02), (LABETALOL\LABETALOL HYDROCHLORIDE:Bronchopulmonary aspergillosis:0.09), (LERCANIDIPINE:Bronchopulmonary aspergillosis:0.44), (LERCANIDIPINE HYDROCHLORIDE:Bronchopulmonary aspergillosis:0.26), (LUMACAFTOR:Bronchopulmonary aspergillosis:0.08), (MEPOLIZUMAB:Bronchopulmonary aspergillosis:0.39), (MOGAMULIZUMAB:Bronchopulmonary aspergillosis:0.05), (NILOTINIB:Bronchopulmonary aspergillosis:0.89), (OLOPATADINE:Bronchopulmonary aspergillosis:0.2), (OXCARBAZEPINE:Bronchopulmonary aspergillosis:0.28), (PANTOPRAZOLE MAGNESIUM:Bronchopulmonary aspergillosis:0.49), (PEGASPARGASE:Bronchopulmonary aspergillosis:0.92), (PENTOSTATIN:Bronchopulmonary aspergillosis:0.1), (POLYETHYLENE GLYCOLS:Bronchopulmonary aspergillosis:0.98), (POLYMYXIN B SULFATE:Bronchopulmonary aspergillosis:0.1), (RANIMUSTINE:Bronchopulmonary aspergillosis:0.02), (RANITIDINE:Bronchopulmonary aspergillosis:0.07), (RANOLAZINE:Bronchopulmonary aspergillosis:0.67), (SACUBITRIL\VALSARTAN:Bronchopulmonary aspergillosis:0.38), (SALMETEROL:Bronchopulmonary aspergillosis:0.13), (SECUKINUMAB:Bronchopulmonary aspergillosis:0.48), (SODIUM BICARBONATE:Bronchopulmonary aspergillosis:0.15), (TAZOBACTAM:Bronchopulmonary aspergillosis:0.12), (TELMISARTAN:Bronchopulmonary aspergillosis:0.55), (TEMAZEPAM:Bronchopulmonary aspergillosis:0.21), (TOPIRAMATE:Bronchopulmonary aspergillosis:0.31), (VINDESINE SULFATE:Bronchopulmonary aspergillosis:0.15), (IVACAFTOR:Bronchopulmonary aspergillosis allergic:0.01), (PYRAZINAMIDE:Bronchoscopy:0.56), (ACETAMINOPHEN\OXYCODONE HYDROCHLORIDE:Bronchospasm:0.67), (AMIODARONE HYDROCHLORIDE:Bronchospasm:0.49), (AMPICILLIN:Bronchospasm:0.02), (ATOVAQUO NE:Bronchospasm:0.03), (BENAZEPRIL HYDROCHLORIDE:Bronchospasm:0.09), (BUMETANIDE:Bronchospasm:1.18), (BUSPIRON E HYDROCHLORIDE:Bronchospasm:0.73), (CALCITRIOL:Bronchospasm:0.58), (CALCIUM ACETATE:Bronchospasm:0.63), (CANAKINUMAB:Bronchospasm:0.14), (CARFILZOMIB:Bronchospasm: 0.6), (CEFOXITIN\CEFOXITIN SODIUM:Bronchospasm:0.21), (CERTOLIZUMAB PEGOL:Bronchospasm:0.97), (CHLORPHENIRAMINE\HYDROCODONE:Bronchospasm:0.04), (CHOLESTYRAMINE:Bronchospasm:0.33), (CLINDAMYCIN\CLINDAMYCIN PHOSPHATE:Bronchospasm:1.47), (CLOZAPINE:Bronchospasm:0.01), (CYPROHEPTADINE: Bronchospasm:0.11), (DACLIZUMAB:Bronchospasm:0.2), (DALFAMPRIDINE:Bronchospasm: 0.23), (DASATINIB:Bronchospasm:0.83), (DEOXYRIBONUCLIEC ACID:Bronchospasm:0.03), (DOXAZOSIN\DOXAZOSIN MESYLATE:Bronchospasm:0.17), (DUTASTERIDE:Bronchospasm:0.29), (ELETRIPTAN HYDROBROMIDE:Bronchospasm:0.35), (ENALAPRIL MALEATE:Bronchospasm:0.35), (EPLERENONE:Bronchospasm:0.05), (EVOLOCUMAB:Bronchospasm: 0.64), (EZETIMIBE\SIMVASTATIN:Bronchospasm:0.28), (FESOTERODINE FUMARATE:Bronchospasm:0.21), (FLECAINIDE:Bronchospasm:0.77), (FORMOTEROL:Bronchospasm: 0.5), (FORMOTEROL FUMARATE\MOMETASONE FUROATE:Bronchospasm:1.46), (FOSFOMYCIN:Bronchospasm:0.02), (HYDROCHLOROTHI AZIDE\LISINOPRIL:Bronchospasm:0.49), (HYDROCHLOROTHIAZIDE\VALSARTAN:Bronchospasm: 0.64), (HYDROXYCHLOROQUINE DIPHOSPHATE:Bronchospasm:0.1), (HYDROXYZINE PAMOATE:Bronchospasm:0.48), (ILOPROST:Bronchospasm:0.73), (IMATINIB MESYLATE:Bronchospasm:0.61), (IMIPRAMINE HYDROCHLORIDE:Bronchospasm:0.19), (INDAPAMIDE:Bronchospasm:0.03), (INDAPAMI DE\PERINDOPRIL:Bronchospasm:0.25), (INDOMETHACIN:Bronchospasm:0.1), (LATANOP ROST:Bronchospasm:0.13), (LEDIPASVIR\SOFOSBUVIR:Bronchospasm:0.14), (LERCANIDI PINE:Bronchospasm:0.01), (LERCANIDIPINE HYDROCHLORIDE:Bronchospasm:0.16), (LIDOCAINE\PRILOCAINE:Bronchospasm:0.15), (LITHIUM CARBONATE:Bronchospasm:0.1), (LUMACAFTOR:Bronchospasm:0.74), (METHENAMINE HIPPURATE:Bronchospasm:0.03), (METHYLPHENIDATE HYDROCHLORIDE:Bronchospasm:0.22), (METHYLPREDNISOLONE ACETATE:Bronchospasm:0.08), (METOLAZONE:Bronchospasm:0.1), (MICAFUNGIN:Bronchospasm: 0.52), (NALOXONE HYDROCHLORIDE\OXYCODONE HYDROCHLORIDE:Bronchospasm:0.23), (NEBIVOLOL:Bronchospasm:0.3), (NILOTINIB:Bronchospasm: 0.69), (OLANZAPINE:Bronchospasm:0.62), (OLOPATADINE:Bronchospasm:0.36), (OXYBUTYNIN CHLORIDE:Bronchospasm:0.1), (PANCRELIPASE\PANCRELIPASE AMYLASE\PANCRELIPASE LIPASE\PANCRELIPASE PROTEASE:Bronchospasm:0.09), (PANTOPRAZOLE MAGNESIUM:Bronchospasm:1.73), (PAROXETINE\PAROXETINE HYDROCHLORIDE:Bronchospasm:0.4), (PAZOPANIB HYDROCHLORIDE:Bronchospasm:0.05), (POLYETHYLENE GLYCOLS:Bronchospasm:1.19), (PRIMIDONE:Bronchospasm:0.71), (RISEDRONATE SODIUM:Bronchospasm:0.65), (SULFAMETHOXAZOLE:Bronchospasm:0.31), (SUMATRIPT AN\SUMATRIPTAN SUCCINATE:Bronchospasm:0.43), (TAMSULOSIN HYDROCHLORIDE:Bronchospasm:0.34), (TERIFLUNOMIDE:Bronchospasm:0.02), (THALID OMIDE:Bronchospasm:0.35), (TIMOLOL:Bronchospasm:0.1), (TOCILIZUMAB:Bronchospasm: 0.06), (TOPIRAMATE:Bronchospasm:1.33), (VANCOMYCIN HYDROCHLORIDE:Bronchospasm:0.73), (ZILEUTON:Bronchospasm:0.03), (ZONISAMIDE: Bronchospasm:0.41), (ALBUTEROL SULFATE\IPRATROPIUM BROMIDE:Burning sensation:0.12), (AMIODARONE:Burning sensation:0.35), (AMIODARONE HYDROCHLORIDE:Burning sensation:0),(ARMODAFINIL:Burning sensation:0.59), (BISACODYL OR DOCUSATE SODIUM:Burning sensation:0.48), (CALCIUM CITRATE:Burning sensation:0.09), (CARVEDILOL PHOSPHATE:Burning sensation:0.24), (CERTOLIZUMAB PEGOL:Burning sensation:1.25), (CLOTRIMAZOLE:Burning sensation:0.07), (DASATINIB:Burning sensation:0.26), (DEVICE:Burning sensation:0.36), (DOCUSATE:Burning sensation:0.02), (ELETRIPTAN HYDROBROMIDE:Burning sensation:0.26), (ENOXAPARIN:Burning sensation:0.19), (ERGOCALCIFEROL:Burning sensation:0.54), (ESZOPICLONE:Burning sensation:0.69), (EVOLOCUMAB:Burning sensation:1.09), (FESOTERODINE FUMARATE:Burning sensation:0), (FLUTICASONE:Burning sensation:0.01), (HYDROXYCHLOROQUINE DIPHOSPHATE:Burning sensation:0.2), (ILOPROST:Burning sensation:0.01), (INDOMETHACIN:Burning sensation:0.19), (LISDEXAMFETAMINE DIMESYLATE:Burning sensation:0.23), (METHYLPHENIDATE HYDROCHLORIDE:Burning sensation:0.04), (NILOTINIB:Burning sensation:0.19), (NITROFURANTOIN:Burning sensation:0.46), (OLANZAPINE:Burning sensation:0.32), (PENICILLIN:Burning sensation:0.11), (RIZATRIPTAN BENZOATE:Burning sensation:0.23), (SACUBITRIL\VALSARTAN:Burning sensation:0.53), (SUMATRIPTAN:Burning sensation:0.25), (TETRACYCLINE:Burning sensation:0.13), (TIOTROPIUM BROMIDE MONOHYDRATE:Burning sensation:0.64), (TRIAMTERENE:Burning sensation:0.16), (VITAMIN B:Burning sensation:0.64), (ANASTROZOLE:Bursitis:0.08), (CIPROFLOXACIN HYDROCHLORIDE:Bursitis:0.05), (CLONIDINE:Bursitis:0.01), (DIPHENHYDRAMINE:Bursitis: 0.52), (ENOXAPARIN:Bursitis:0.17), (FERROUS SULFATE:Bursitis:0.24), (FERROUS SULFATE\FERROUS SULFATE, DRIED:Bursitis:0.5), (HYDROXYCHLOROQUINE DIPHOSPHATE:Bursitis:0.13), (NIFEDIPINE:Bursitis:0.1), (OXCARBAZEPINE:Bursitis:0.26), (OXYGEN:Bursitis:0.4), (PANTOPRAZOLE:Bursitis:0.75), (PAROXETINE:Bursitis:0.1), (PRA MIPEXOLE DIHYDROCHLORIDE:Bursitis:0.14), (PRAVASTATIN SODIUM:Bursitis:0.24), (RANOLAZINE:Bursitis:0.21), (SACUBITRIL\VALSARTAN:Bursitis: 0.6), (TAMSULOSIN:Bursitis:0.75), (TEMAZEPAM:Bursitis:0.03), (TERIFLUNOMIDE:Bursitis: 0.74), (VITAMIN B:Bursitis:0.06), (ZOLPIDEM\ZOLPIDEM TARTRATE:Bursitis:0.1), (AMIODARONE HYDROCHLORIDE:Calcium deficiency:0.78), (AMPHETAMINE ASPARTATE\AMPHETAMINE SULFATE\DEXTROAMPHETAMINE SACCHARATE\DEXTROAMPHETAMINE SULFATE:Calcium deficiency:0.27), (BISACODYL OR DOCUSATE SODIUM:Calcium deficiency:0.3), (BORTEZOMIB:Calcium deficiency:0.91), (CANDESARTAN:Calcium deficiency:0.59), (CANDESARTAN CILEXETIL:Calcium deficiency:0.64), (CHOLESTYRAMINE:Calcium deficiency:0.78), (CLOTRIMAZOLE:Calcium deficiency:0.4), (CLOZAPINE:Calcium deficiency:0.25), (COLCHICINE:Calcium deficiency:0.58), (DALFAMPRIDINE:Calcium deficiency:0.22), (DASATINIB:Calcium deficiency:0.67), (DEVICE:Calcium deficiency:0.14), (DICLOFENAC:Calcium deficiency:0.65), (DIVALPROEX SODIUM:Calcium deficiency:0.28), (DOFETILIDE:Calcium deficiency:0.09), (DOXAZOSIN\DOXAZOSIN MESYLATE:Calcium deficiency:0.24), (DUTASTERIDE:Calcium deficiency:0.17), (ENALAPRIL MALEATE:Calcium deficiency:0.27), (ESZOPICLONE:Calcium deficiency:0.69), (EVOLOCUMAB:Calcium deficiency:0.68), (FEBUXOSTAT:Calcium deficiency:0.1), (HYDROCHLOROTHIAZIDE\IRBESARTAN:Calcium deficiency:0.25), (HYDROMORPHONE:Calcium deficiency:0.32), (HYDROMORPHONE HYDROCHLORIDE:Calcium deficiency:0.39), (HYDROXYCHLOROQUINE DIPHOSPHATE:Calcium deficiency:0.03), (HYDROXYZINE PAMOATE:Calcium deficiency:0.09), (ILOPROST:Calcium deficiency:0.08), (IMATINIB MESYLATE:Calcium deficiency:0.62), (INDAPAMIDE\PERINDOPRIL:Calcium deficiency:0.42), (INFLUENZA VIRUS VACCINE:Calcium deficiency:0.16), (LATANOPROST:Calcium deficiency:0), (LEVALBUTEROL HYDROCHLORIDE:Calcium deficiency:0.1), (MELATONIN:Calcium deficiency:0.17), (METHADONE HYDROCHLORIDE:Calcium deficiency:0.26), (METHYLPREDNISOLONE ACETATE:Calcium deficiency:0.25), (METRONIDAZOLE:Calcium deficiency:1.57), (METRONIDAZOLE\METRONIDAZOLE HYDROCHLORIDE:Calcium deficiency:0.21), (MOXIFLOXACIN HYDROCHLORIDE:Calcium deficiency:0.21), (NADOLOL:Calcium deficiency:0.06), (NIACIN:Calcium deficiency:0.07), (NILOTINIB:Calcium deficiency:0.59), (OMEGA-3-ACID ETHYL ESTERS:Calcium deficiency:0.31), (OXCARBAZEPINE:Calcium deficiency:0.6), (PAMIDRONATE DISODIUM:Calcium deficiency:0.1), (PANTOPRAZOLE MAGNESIUM:Calcium deficiency:0.68), (PAZOPANIB HYDROCHLORIDE:Calcium deficiency:0.03), (PEGFILGRASTIM:Calcium deficiency:0.09), (PERINDOPRIL:Calcium deficiency:0.29), (PRAMIPEXOLE:Calcium deficiency:0.05), (PRIMIDONE:Calcium deficiency:0.33), (QUETIAPINE:Calcium deficiency:0.12), (RABEPRAZOLE:Calcium deficiency:0.06), (RALOXIFENE HYDROCHLORIDE:Calcium deficiency:0.29), (RANOLAZINE:Calcium deficiency:0.87), (SACUBITRIL\VALSARTAN:Calcium deficiency:0.69), (SECUKINUMAB:Calcium deficiency:0.2), (TERIFLUNOMIDE:Calcium deficiency:0.1), (TOLTERODINE TARTRATE:Calcium deficiency:0.06), (TRIAMTERENE:Calcium deficiency:0.14), (UMECLIDINIUM BROMIDE\VILANTEROL TRIFENATATE:Calcium deficiency:0.26), (.ALPHA.-TOCOPHEROL\RETINOL:Cancer pain:0.6), (ACETAMINOPHEN\APRONALIDE\CAFFEINE\PROPYPHENAZONE:Cancer pain:0.6), (AGALSIDASE BETA:Cancer pain:0.11), (ALDESLEUKIN:Cancer pain:0.06), (ALENDRONIC ACID:Cancer pain:1.31), (ALFUZOSIN HYDROCHLORIDE:Cancer pain:0.22), (AMEZINIUM METILSULFATE:Cancer pain:1.31), (AMIODARONE HYDROCHLORIDE:Cancer pain:0.87), (AMISULPRIDE:Cancer pain:0.93), (AMLODIPINE BESYLATE\CANDESARTAN CILEXETIL:Cancer pain:0.85), (AMLODIPINE\VALSARTAN:Cancer pain:0.16), (ANAGLIPTIN:Cancer pain:0.49), (ATROPINE:Cancer pain:0.16), (BACITRACIN:Cancer pain:0.07), (BECLOMETHASONE DIPROPIONATE:Cancer pain:0.79), (BENAZEPRIL\BENAZEPRIL HYDROCHLORIDE:Cancer pain:0.38), (BENDAMUSTINE HYDROCHLORIDE:Cancer pain:0.25), (BERAPROST:Cancer pain:0.09), (BETAMETHASONE\DEXCHLORPHENIRAMINE MALEATE:Cancer pain:0.03), (BETAMIPRON\PANIPENEM:Cancer pain:0.09), (BICARBONATE ION\POLYETHYLENE GLYCOLS\POTASSIUM CATION\SODIUM CHLORIDE\SODIUM SULFATE ANHYDROUS:Cancer pain:0.31), (BUCOLOME:Cancer pain:0.02), (BUFORMIN:Cancer pain:0.05), (CALCITONIN SALMON:Cancer pain:1.06), (CALCIUM ACETATE:Cancer pain:0.68), (CALCIUM CITRATE:Cancer pain:0.76), (CANAGLIFLOZIN:Cancer pain:0.02), (CANAKINUMAB:Cancer pain:0.35), (CARPERITIDE:Cancer pain:0.58), (CARVEDILOL PHOSPHATE:Cancer pain:0.86), (CEFPIROME SULFATE:Cancer pain:0.05), (CERTOLIZUMAB PEGOL:Cancer pain:1.28), (CHLORAMBUCIL:Cancer pain:0.18), (CHLORHEXIDINE:Cancer pain:0.45), (CHLORPROMAZINE\PROMETHAZINE:Cancer pain:0.14), (CHLORTHALIDONE:Cancer pain:0.33), (CHOLESTYRAMINE:Cancer pain:0.97), (CICLESONIDE:Cancer pain:0.04), (CLENBUTEROL HYDROCHLORIDE:Cancer pain:0.35), (CLOTRIMAZOLE:Cancer pain:0.58), (COLCHICINE:Cancer pain:0.73), (COLESTILAN CHLORIDE:Cancer pain:0.23), (CROTAMITON:Cancer pain:0.7), (CYANOCOBALAMIN\LIDOCAINE HYDROCHLORIDE\PYRIDOXINE HYDROCHLORIDE\THIAMINE HYDROCHLORIDE:Cancer pain:0.23), (DABIGATRAN:Cancer pain:0), (DABRAFENIB:Cancer pain:0.95), (DACLIZUMAB:Cancer pain:0.06), (DANAZOL:Cancer pain:0.14), (DANTROLENE SODIUM:Cancer pain:0.08), (DEFLAZACORT:Cancer pain:0.42), (DEVICE:Cancer pain:0.06), (DIFLUCORTOLONE VALERATE\LIDOCAINE:Cancer pain:0.15), (DIGITOXIN:Cancer pain:0.43), (DIMEMORFAN PHOSPHATE:Cancer pain:0.68), (DIQUAFOSOL TETRASODIUM:Cancer pain:0.53), (DOFETILIDE:Cancer pain:0.06), (DOLASETRON MESYLATE:Cancer pain:0.32), (DOXEPIN HYDROCHLORIDE:Cancer pain:0.15), (ECABET:Cancer pain:0.18), (ECABET SODIUM:Cancer pain:0.4), (ELBASVIR:Cancer pain:0.05), (ELCATONIN:Cancer pain:0.69), (ELETRIPTAN HYDROBROMIDE:Cancer pain:0.67), (ELOTUZUMAB:Cancer pain:0.18), (EPHEDRINE HYDROCHLORIDE:Cancer pain:0.09), (EPRAZINONE:Cancer pain:0.14), (ERYTHROMYCIN LACTOBIONATE:Cancer pain:0.34), (ETHYL ICOSAPENTATE\ICOSAPENT:Cancer pain:0.78), (ETRETINATE:Cancer pain:1.8), (FERROUS SULFATE\GLYCINE:Cancer pain:0.02), (FLUOCINONIDE:Cancer pain:0.15), (FLUTICASONE:Cancer pain:0.75), (FLUTICASONE PROPIONATE\FORMOTEROL FUMARATE:Cancer pain:0.01), (FORMOTEROL FUMARATE:Cancer pain:0.7), (FORMOTEROL FUMARATE DIHYDRATE\MOMETASONE FUROATE:Cancer pain:0.54), (FOSFOMYCIN CALCIUM:Cancer pain:0.32), (FOSPHENYTOIN\FOSPHENYTOIN SODIUM:Cancer pain:0.32), (FURSULTIAMINE:Cancer pain:0.65), (FUSIDIC ACID:Cancer pain:0.31), (GEMFIBROZIL:Cancer pain:0.12), (GLYCOPYRROLATE:Cancer pain:0.35), (GRAZOPREVIR:Cancer pain:0.03), (GUAIAZULENE:Cancer pain:0.15), (GUANABENZ ACETATE:Cancer pain:0.43), (HERBALS\TURMERIC:Cancer pain:0.13), (HYDROCHLOROTHIAZIDE\IRBESARTAN:Cancer pain:0.83), (HYDROCHLOROTHIAZIDE\TELMISARTAN:Cancer pain:0.24), (HYDROCORTISONE PROBUTATE:Cancer pain:0.01), (IDELALISIB:Cancer pain:0.12), (IGURATIMOD:Cancer pain:1.09), (ILOPROST:Cancer pain:0.41), (IMATINIB MESYLATE:Cancer pain:1.75), (IMIDAFENACIN:Cancer pain:0.22), (IMIDAPRIL HYDROCHLORIDE:Cancer pain:0.33), (INTERFERON ALFA-2B:Cancer pain:0.56), (INTERFERON ALFA-N1:Cancer pain:0.18), (IRBESARTAN\TRICHLORMETHIAZIDE:Cancer pain:0.06), (ITOPRIDE:Cancer pain:0.27), (LABETALOL\LABETALOL HYDROCHLORIDE:Cancer pain:1.36), (LACTOBACILLUS RHAMNOSUS:Cancer pain:0), (LANDIOLOL HYDROCHLORIDE:Cancer pain:0.82), (LAPATINIB:Cancer pain:0.26), (LEDIPASVIR\SOFOSBUVIR:Cancer pain:0.49), (LERCANIDIPINE:Cancer pain:0.51), (LERCANIDIPINE HYDROCHLORIDE:Cancer pain:0.1), (LEVOCABASTINE HYDROCHLORIDE:Cancer pain:1.08), (LISDEXAMFETAMINE DIMESYLATE:Cancer pain:0.83), (LOVASTATIN:Cancer pain:0.81), (LYSOZYME HYDROCHLORIDE:Cancer pain:0.61), (MAXACALCITOL:Cancer pain:0.97), (MECLIZINE HYDROCHLORIDE:Cancer pain:0.13), (MECLIZINE\MECLIZINE HYDROCHLORIDE:Cancer pain:0.61), (MEPENZOLATE BROMIDE:Cancer pain:0.32), (METAXALONE:Cancer pain:0.68), (METHENAMINE HIPPURATE:Cancer pain:0.71), (METHENOLONE ACETATE:Cancer pain:0.01), (METHENOLONE ENANTHATE:Cancer pain:1.59), (METHYLPREDNISOLONE ACETATE:Cancer pain:0.46), (MITIGLINIDE CALCIUM HYDRATE\VOGLIBOSE:Cancer pain:0.56), (MIZORIBINE:Cancer pain:0.94), (NAFAMOSTAT MESYLATE:Cancer pain:0.14), (NIACIN:Cancer pain:0.52), (NILVADIPINE:Cancer pain:0.23), (NIPRADILOL:Cancer pain:0.16), (NITROFURANTOIN\NITROFURANTOIN MONOHYDRATE:Cancer pain:0.03), (NIZATIDINE:Cancer pain:0.02), (OMEPRAZOLE SODIUM:Cancer pain:0.01), (PANTOPRAZOLE MAGNESIUM:Cancer pain:0.99), (PAZUFLOXACIN MESILATE:Cancer pain:0.19), (PIRARUBICIN HYDROCHLORIDE:Cancer pain:0.03), (PLANTAGO SEED:Cancer pain:0.23), (POTASSIUM CHLORIDE\SODIUM CHLORIDE\SODIUM LACTATE:Cancer pain:0.43), (POTASSIUM IODIDE:Cancer pain:0.11), (PRAZOSIN:Cancer pain:0.33), (PREDNISOLONE VALERATE ACETATE:Cancer pain:0.33), (PROGESTERONE:Cancer pain:0.4), (PYRIDOXINE:Cancer pain:0.4), (PYRIDOXINE HYDROCHLORIDE:Cancer pain:0.46), (QUERCUS SALICINA STEM:Cancer pain:0.11), (RIVASTIGMINE:Cancer pain:0.27), (ROPINIROLE:Cancer pain:0.52), (ROXATIDINE ACETATE HYDROCHLORIDE:Cancer pain:0.24), (SACUBITRIL\VALSARTAN:Cancer pain:0.34), (SARPOGRELATE HYDROCHLORIDE:Cancer pain:0.99), (SECUKINUMAB:Cancer pain:0.17), (SEVELAMER HYDROCHLORIDE:Cancer pain:0.15), (SIPULEUCEL-T:Cancer pain:0.05), (SIVELESTAT SODIUM:Cancer pain:0.58), (SODIUM OXYBATE:Cancer pain:0.97), (SUFENTANIL CITRATE:Cancer pain:0.24), (SUMATRIPTAN\SUMATRIPTAN SUCCINATE:Cancer pain:0.41), (SUPLATAST TOSILATE:Cancer pain:0.54), (TECELEUKIN:Cancer pain:0.27), (TERAZOSIN HYDROCHLORIDE:Cancer pain:0.19), (TERBUTALINE SULFATE:Cancer pain:0.36), (THIAMYLAL SODIUM:Cancer pain:0.18), (TIARAMIDE:Cancer pain:0.05), (TIMOLOL:Cancer pain:0.77), (TINZAPARIN:Cancer pain:0.26), (TOCOPHEROL:Cancer pain:0.01), (TOREMIFENE:Cancer pain:0.14), (TRAMETINIB DIMETHYL SULFOXIDE:Cancer pain:0.01), (VALGANCICLOVIR HYDROCHLORIDE:Cancer pain:0.17), (VILAZODONE HYDROCHLORIDE:Cancer pain:0.13), (VITAMIN A:Cancer pain:0.15), (VITAMIN B:Cancer pain:0.65), (VONOPRAZAN:Cancer pain:0.68), (ZIV-AFLIBERCEPT:Cancer pain:0.1), (ZOTEPINE:Cancer pain:0.09), (ACARBOSE:Candida infection:0.38), (ACENOCOUMAROL:Candida infection:0.2), (ACETAMINOPHEN\BUTALBITAL\CAFFEINE:Candida infection:1.18), (ACETAMINOPHEN\OXYCODONE\OXYCODONE HYDROCHLORIDE:Candida infection:0.48), (ACETAMINOPHEN\PROPOXYPHENE HYDROCHLORIDE:Candida infection:0.1), (ADO-TRASTUZUMAB EMTANSINE:Candida infection:0.74), (AGALSIDASE BETA:Candida infection:0.06), (ALCOHOL:Candida infection:0.22), (ALEMTUZUMAB:Candida infection:0.31), (ALENDRONIC ACID:Candida infection:0.66), (ALUMINUM HYDROXIDE\DIMETHICONE\MAGNESIUM HYDROXIDE:Candida infection:0.43), (AMANTADINE:Candida infection:0.15), (AMISULPRIDE:Candida infection:0.22), (AMLODIPINE\VALSARTAN:Candida infection:0.04), (ANTIHISTAMINES NOS:Candida infection:0.03), (AZELASTINE:Candida infection:0.26), (BELATACEPT:Candida infection:0.36), (BLINATUMOMAB:Candida infection:1.01), (BRINZOLAMIDE:Candida infection:0.26), (BUPRENORPHINE HYDROCHLORIDE:Candida infection:0.27), (BUPROPION:Candida infection:1.02), (CALCITRIOL:Candida infection:1.3), (CAPTOPRIL:Candida infection:0.3), (CARVEDILOL PHOSPHATE:Candida infection:0.5), (CEFOTAXIME\CEFOTAXIME SODIUM:Candida infection:0.51), (CHOLESTYRAMINE:Candida infection:1.57), (CLOBETASOL:Candida infection:0.28), (CORTISONE\HYDROCORTISONE:Candida infection:0.95), (CRIZOTINIB:Candida infection:0.24), (CYPROHEPTADINE:Candida infection:0.35), (CYPROHEPTADINE HYDROCHLORIDE:Candida infection:0.18), (DABRAFENIB:Candida infection:0.37), (DACLIZUMAB:Candida infection:1.39), (DALFAMPRIDINE:Candida infection:0.14), (DANAZOL:Candida infection:0.24), (DASATINIB:Candida infection:1.47), (DEFLAZACORT:Candida infection:0.3), (DESLORATADINE:Candida infection:0.9), (DESONIDE:Candida infection:0.14), (DEVICE:Candida infection:0.09), (DEXAMETHASONE ACETATE:Candida infection:0.6), (DEXLANSOPRAZOLE:Candida infection:1.57), (DIGITOXIN:Candida infection:0.02), (DIPYRIDAMOLE:Candida infection:0.21), (DOFETILIDE:Candida infection:0.36), (DOLASETRON MESYLATE:Candida infection:0.13), (ELETRIPTAN HYDROBROMIDE:Candida infection:0.93), (ELOTUZUMAB:Candida infection:0.61), (EPIRUBICIN:Candida infection:0.42), (EVOLOCUMAB:Candida infection:0.35), (FENOTEROL HYDROBROMIDE:Candida infection:0.01), (FENOTEROL HYDROBROMIDE\IPRATROPIUM BROMIDE:Candida infection:0.26), (FEXOFENADINE HYDROCHLORIDE:Candida infection:0.31), (FINGOLIMOD HYDROCHLORIDE:Candida infection:0.18), (FLUDROCORTISONE ACETATE:Candida infection:0.17), (FORMOTEROL FUMARATE:Candida infection:1.08), (FORMOTEROL FUMARATE\MOMETASONE FUROATE:Candida infection:0.8), (FULVESTRANT:Candida infection:0.88), (GLYCOPYRRONIUM\INDACATEROL:Candida infection:0.85), (HERBALS\TURMERIC:Candida infection:0.28), (HYDROCHLOROTHIAZIDE\IRBESARTAN:Candida infection:0.81), (HYDROCHLOROTHIAZIDE\TELMISARTAN:Candida infection:0.13), (HYDROXYCHLOROQUINE DIPHOSPHATE:Candida infection:0.34), (IBRITUMOMAB TIUXETAN:Candida infection:0.75), (IDARUBICIN:Candida infection:0.26), (ILOPROST:Candida infection:0.32), (IMATINIB MESYLATE:Candida infection:0.73), (IMIPRAMINE HYDROCHLORIDE:Candida infection:0.34), (INDAPAMIDE\PERINDOPRIL:Candida infection:1.15), (IRINOTECAN:Candida infection:0.35), (IRINOTECAN HYDROCHLORIDE:Candida infection:0.26), (LABETALOL HYDROCHLORIDE:Candida infection:0.45), (LACTOBACILLUS RHAMNOSUS:Candida infection:0.24), (LAPATINIB DITOSYLATE:Candida infection:0.06), (LEDIPASVIR\SOFOSBUVIR:Candida infection:0.34), (LINAGLIPTIN:Candida infection:0.73), (LITHIUM CARBONATE:Candida infection:1.26), (MEGESTROL:Candida infection:0.06), (MEPOLIZUMAB:Candida infection:0.45), (METAXALONE:Candida infection:1.04), (METHENAMINE HIPPURATE:Candida infection:0.8), (METHENOLONE ENANTHATE:Candida infection:0.04), (METHYLPREDNISOLONE ACETATE:Candida infection:1.08), (MIRABEGRON:Candida infection:0), (NICARDIPINE:Candida infection:0.18), (NILOTINIB:Candida infection:1.59), (NITROFURANTOIN\NITROFURANTOIN MONOHYDRATE:Candida infection:0.12), (NOREPINEPHRINE:Candida infection:0.53), (NORETHINDRONE:Candida infection:0.03), (OLAPARIB:Candida infection:0.03), (OLOPATADINE:Candida infection:0.49), (OMEGA-3-ACID ETHYL ESTERS:Candida infection:0.71), (OXCARBAZEPINE:Candida infection:1.67), (PAROXETINE:Candida infection:0.71), (PAROXETINE\PAROXETINE HYDROCHLORIDE:Candida infection:0.93), (PEMETREXED DISODIUM:Candida infection:0.66), (PENTOSTATIN:Candida infection:0.28), (PHENOBARBITAL:Candida infection:0.72), (PIROXICAM:Candida infection:0.18), (PITAVASTATIN CALCIUM:Candida infection:0.31), (PONATINIB:Candida infection:0.79), (PRAMIPEXOLE DIHYDROCHLORIDE:Candida infection:0.62), (PRAVASTATIN\PRAVASTATIN SODIUM:Candida infection:0.42), (PREDNISOLONE SODIUM PHOSPHATE:Candida infection:0.51), (PRIMIDONE:Candida infection:1), (PYRIDOXINE:Candida infection:0.46), (RADIATION THERAPY:Candida infection:0.17), (RANOLAZINE:Candida infection:0.95), (REGORAFENIB:Candida infection:0.15), (SACUBITRIL\VALSARTAN:Candida infection:0.53), (SALMETEROL:Candida infection:0.2), (SOTALOL:Candida infection:0.93), (TELMISARTAN:Candida infection:1.13), (TERAZOSIN HYDROCHLORIDE:Candida infection:0.13), (TERIFLUNOMIDE:Candida infection:0.22), (THEOPHYLLINE:Candida infection:0.17), (TINZAPARIN:Candida infection:0.37), (TOCOPHEROL:Candida infection:0.03), (TRAMETINIB:Candida infection:0.48), (AMLODIPINE BESYLATE\HYDROCHLOROTHIAZIDE\OLMESARTAN MEDOXOMIL:Carcinoid syndrome:0.37), (BISACODYL OR DOCUSATE SODIUM:Carcinoid syndrome:0.54), (BISOPROLOL FUMARATE:Carcinoid syndrome:1.21), (BROMAZEPAM:Carcinoid syndrome:0.06), (CARVEDILOL PHOSPHATE:Carcinoid syndrome:0.2), (CERTOLIZUMAB PEGOL:Carcinoid syndrome:0.43), (CINACALCET HYDROCHLORIDE:Carcinoid syndrome:0.01), (CLINDAMYCIN\CLINDAMYCIN PHOSPHATE:Carcinoid syndrome:0.68), (CORTISONE\HYDROCORTISONE:Carcinoid syndrome:0.12), (DASATINIB:Carcinoid syndrome:0.67), (DEVICE:Carcinoid syndrome:0.02), (DOFETILIDE:Carcinoid syndrome:0.01), (ELETRIPTAN HYDROBROMIDE:Carcinoid syndrome:0.35), (ERYTHROPOIETIN:Carcinoid syndrome:0.26), (ESZOPICLONE:Carcinoid syndrome:0.58), (EVOLOCUMAB:Carcinoid syndrome:0.62), (EZETIMIBE:Carcinoid syndrome:0.53), (FLECAINIDE:Carcinoid syndrome:0.19), (HYDROCHLOROTHIAZIDE\IRBESARTAN:Carcinoid syndrome:0.15), (HYDROCHLOROTHIAZIDE\LISINOPRIL:Carcinoid syndrome:0.44), (HYDROXYZINE PAMOATE:Carcinoid syndrome:0.15), (IBRUTINIB:Carcinoid syndrome:0.83), (IPRATROPIUM:Carcinoid syndrome:0.1), (LEDIPASVIR\SOFOSBUVIR:Carcinoid syndrome:0.14), (LENALIDOMIDE:Carcinoid syndrome:0.2), (LERCANIDIPINE HYDROCHLORIDE:Carcinoid syndrome:0.15), (LINAGLIPTIN:Carcinoid syndrome:0.51), (LISDEXAMFETAMINE DIMESYLATE:Carcinoid syndrome:0.04), (METHYLPREDNISOLONE ACETATE:Carcinoid syndrome:0.25), (MODAFINIL:Carcinoid syndrome:0), (NADOLOL:Carcinoid syndrome:0.13), (NEBIVOLOL:Carcinoid syndrome:0.18), (NILOTINIB:Carcinoid syndrome:0.01), (OXYGEN:Carcinoid syndrome:0.38), (PANTOPRAZOLE MAGNESIUM:Carcinoid syndrome:0.37), (PAROXETINE\PAROXETINE HYDROCHLORIDE:Carcinoid syndrome:0.04), (PRAMIPEXOLE DIHYDROCHLORIDE:Carcinoid syndrome:0.35), (PRIMIDONE:Carcinoid syndrome:0.7), (QUETIAPINE FUMARATE:Carcinoid syndrome:0.21), (RABEPRAZOLE SODIUM:Carcinoid syndrome:0.08), (RANOLAZINE:Carcinoid syndrome:0.91), (TERIFLUNOMIDE:Carcinoid syndrome:0.21), (TETRACYCLINE:Carcinoid syndrome:0.66), (TOFACITINIB CITRATE:Carcinoid syndrome:0.02), (TRIAMCINOLONE ACETONIDE:Carcinoid syndrome:0.06), (UMECLIDINIUM BROMIDE\VILANTEROL TRIFENATATE:Carcinoid syndrome:0.35), (VARENICLINE TARTRATE:Carcinoid syndrome:0.43), (VERAPAMIL HYDROCHLORIDE:Carcinoid syndrome:1.16), (ZINC:Carcinoid syndrome:0.03), (ZONISAMIDE:Carcinoid syndrome:0.11), (AMIODARONE HYDROCHLORIDE:Carcinoid tumour:0.57), (AMPHETAMINE ASPARTATE\AMPHETAMINE SULFATE\DEXTROAMPHETAMINE SACCHARATE\DEXTROAMPHETAMINE SULFATE:Carcinoid tumour:0.29), (CARFILZOMIB:Carcinoid tumour:0.07), (CERTOLIZUMAB PEGOL:Carcinoid tumour:0.8), (CLOTRIMAZOLE:Carcinoid tumour:0.35), (DAPAGLIFLOZIN PROPANEDIOL:Carcinoid tumour:0.05), (DEVICE:Carcinoid tumour:0.32), (DICLOFENAC:Carcinoid tumour:0.58), (DICLOFENAC SODIUM:Carcinoid tumour:0.63), (DIPHENHYDRAMINE:Carcinoid tumour:0.75), (DOFETILIDE:Carcinoid tumour:0.15), (DOXAZOSIN MESYLATE:Carcinoid tumour:0.15), (ELETRIPTAN HYDROBROMIDE:Carcinoid tumour:0.29), (ERYTHROPOIETIN:Carcinoid tumour:0.32), (ESOMEPRAZOLE:Carcinoid tumour:0.37), (ESZOPICLONE:Carcinoid tumour:0.51), (EVOLOCUMAB:Carcinoid tumour:0.71), (EZETIMIBE\SIMVASTATIN:Carcinoid tumour:0.14), (FLUCONAZOLE:Carcinoid tumour:0.32), (FLUTICASONE:Carcinoid tumour:0.1), (HERBALS\TURMERIC:Carcinoid tumour:0.02), (HYDROCHLOROTHIAZIDE\IRBESARTAN:Carcinoid tumour:0.13), (IBRUTINIB:Carcinoid tumour:1.58), (ISOSORBIDE:Carcinoid tumour:0.31), (LERCANIDIPINE HYDROCHLORIDE:Carcinoid tumour:0.04), (METAXALONE:Carcinoid tumour:0.24), (METHOCARBAMOL:Carcinoid tumour:0.03), (METHYLPREDNISOLONE ACETATE:Carcinoid tumour:0.04), (NITROFURANTOIN:Carcinoid tumour:0.39), (OMEGA-3-ACID ETHYL ESTERS:Carcinoid tumour:0.35), (PONATINIB:Carcinoid tumour:0.33), (QUETIAPINE FUMARATE:Carcinoid tumour:0.03), (RABEPRAZOLE:Carcinoid tumour:0.15), (RALOXIFENE HYDROCHLORIDE:Carcinoid tumour:0.13), (RANOLAZINE:Carcinoid tumour:0.7), (SODIUM OXYBATE:Carcinoid tumour:0), (TERIFLUNOMIDE:Carcinoid tumour:0.11), (THALIDOMIDE:Carcinoid tumour:0.26), (TOCILIZUMAB:Carcinoid tumour:0.14), (TOFACITINIB CITRATE:Carcinoid tumour:0.21), (TRIAMTERENE:Carcinoid tumour:0.01), (ATRACURIUM:Cardiac arrest:0.11), (ISOFLURANE:Cardiac arrest:0.44), (SUGAMMADEX:Cardiac arrest:0.01), (VECURONIUM BROMIDE:Cardiac arrest:0.32), (. BETA.-CAROTENE:Cardiac disorder:0.49), (2-HYDROXYETHYL METHACRYLATE:Cardiac disorder:0.01), (ABOBOTULINUMTOXINA:Cardiac disorder:0.67), (ACETAMINOPHEN\CAFFEINE:Cardiac disorder:1.26), (ACETAMINOPHEN\CHLORPHENIRAMINE MALEATE\PSEUDOEPHEDRINE HYDROCHLORIDE:Cardiac disorder:0.33), (ACETAMINOPHEN\METHOCARBAMOL:Cardiac disorder:0.62), (ACETYLCARNITINE HYDROCHLORIDE:Cardiac disorder:0.5), (ALBUTEROL\IPRATROPIUM BROMIDE:Cardiac disorder:0.41), (ALFALFA:Cardiac disorder:0.17), (ALGLUCOSIDASE ALFA:Cardiac disorder:0.48), (ALISKIREN HEMIFUMARATE\VALSARTAN:Cardiac disorder:0.26), (ALMOTRIPTAN MALATE:Cardiac disorder:0.25), (AMINO ACIDS\DEXTROSE\ELECTROLYTES NOS:Cardiac disorder:1.2), (AMINO ACIDS\DEXTROSE\ELECTROLYTES NOS\SOYBEAN OIL:Cardiac disorder:0.03), (AMINOSALICYLIC ACID:Cardiac disorder:0.33), (AMMONIUM CHLORIDE\DEXTROMETHORPHAN\SODIUM CITRATE:Cardiac disorder:0.9), (AMOXICILLIN\CLARITHROMYCIN\ESOMEPRAZOLE:Cardiac disorder:0.63), (ANAGRELIDE:Cardiac disorder:0.24), (APREPITANT:Cardiac disorder:1.16), (ARIPIPRAZOLE LAUROXIL:Cardiac disorder:0.71), (ASPERGILLUS NIGER .ALPHA.-GALACTOSIDASE:Cardiac disorder:0.58), (ASUNAPREVIR:Cardiac disorder:0.39), (ATORVASTATIN CALCIUM\EZETIMIBE:Cardiac disorder:0.03), (BACITRACIN ZINC:Cardiac disorder:0.04), (BECLOMETHASONE DIPROPIONATE\FORMOTEROL:Cardiac disorder:0.29), (BERAPROST:Cardiac disorder:0.27), (BERAPROST SODIUM:Cardiac disorder:0.71), (BETHANECHOL CHLORIDE:Cardiac disorder:1.3), (BIFIDOBACTERIUM LONGUM\LACTOBACILLUS ACIDOPHILUS\LACTOBACILLUS RHAMNOSUS:Cardiac disorder:0.29), (BILASTINE:Cardiac disorder:1.01), (BISACODYL\GLYCERIN\MINERAL OIL\SODIUM PHOSPHATE, DIBASIC\SODIUM PHOSPHATE, MONOBASIC:Cardiac disorder:0.11), (BISMUTH SUBSALICYLATE:Cardiac disorder:0.36), (BORON:Cardiac disorder:0.2), (BOSUTINIB:Cardiac disorder:0.33), (BOTULINUM TOXIN TYPE A:Cardiac disorder:0.53), (BROMFENAC SODIUM:Cardiac disorder:0.31), (BUPIVACAINE HYDROCHLORIDE:Cardiac disorder:0.65), (CABOZANTINIB S-MALATE:Cardiac disorder:0.52), (CAJUPUT OIL\EUCALYPTUS OIL\PEPPERMINT OIL:Cardiac disorder:0.22), (CALCIUM CARBONATE\ERGOCALCIFEROL:Cardiac disorder:0.51), (CALCIUM CARBONATE\ETIDRONATE DISODIUM:Cardiac disorder:0.43), (CAMPHOR\DIPHENHYDRAMINE\ZINC OXIDE:Cardiac disorder:0.29), (CARBIMAZOLE:Cardiac disorder:1.27), (CARBOXYMETHYLCELLULOSE SODIUM\HYPROMELLOSES:Cardiac disorder:0.13), (CARTEOLOL:Cardiac disorder:0.21), (CARTEOLOL HYDROCHLORIDE:Cardiac disorder:0.43), (CASPOFUNGIN:Cardiac disorder:0.49), (CEFOTAXIME SODIUM:Cardiac disorder:1.25), (CEFPODOXIME PROXETIL:Cardiac disorder:0.73), (CEFPROZIL:Cardiac disorder:0.17), (CEFTAZIDIME:Cardiac disorder:1.05), (CEFTAZIDIME SODIUM:Cardiac disorder:0.03), (CERITINIB:Cardiac disorder:0.47), (CERTOPARIN SODIUM:Cardiac disorder:0.44), (CHLORDIAZEPOXIDE\CLIDINIUM:Cardiac disorder:0.34), (CHLOROQUINE PHOSPHATE:Cardiac disorder:0.22), (CHOLINE\TRAZODONE HYDROCHLORIDE:Cardiac disorder:0.05), (CILAZAPRIL ANHYDROUS:Cardiac disorder:0.21), (CINACALCET:Cardiac disorder:0.15), (CIPROFLOXACIN\DEXAMETHASONE:Cardiac disorder:0.01), (CLOFAZIMINE:Cardiac disorder:0.31), (CODEINE PHOSPHATE\PROMETHAZINE:Cardiac disorder:0.11), (COLISTIMETHATE SODIUM:Cardiac disorder:0.25), (COLISTIN:Cardiac disorder:0.21), (CYAMEMAZINE:Cardiac disorder:0.35), (DACLIZUMAB:Cardiac disorder:1.92), (DANTROLENE SODIUM:Cardiac disorder:0.77), (DEFEROXAMINE MESYLATE:Cardiac disorder:0), (DEGARELIX:Cardiac disorder:0.26), (DEXAMETHASONE ACETATE:Cardiac disorder:0.86), (DEXAMETHASONE\NEOMYCIN\POLYMYXIN B SULFATE:Cardiac disorder:0.09), (DEXTRAN 70\HYPROMELLOSE 2910 (4000 MPA.S):Cardiac disorder:0.09), (DICLOFENAC DIETHYLAMINE:Cardiac disorder:0.13), (DIFLUPREDNATE:Cardiac disorder:0.33), (DIMETHINDENE:Cardiac disorder:0.28), (DIMETHYL SULFOXIDE:Cardiac disorder:0.55), (DIPHENHYDRAMINE HYDROCHLORIDE\DOXYLAMINE SUCCINATE:Cardiac disorder:0.11), (DOCOSANOL:Cardiac disorder:0.01), (DOLASETRON MESYLATE:Cardiac disorder:1.2), (DOLUTEGRAVIR SODIUM:Cardiac disorder:0.22), (ECONAZOLE:Cardiac disorder:2.03), (EDARAVONE:Cardiac disorder:0.22), (EDOXABAN TOSILATE MONOHYDRATE:Cardiac disorder:0.03), (EMPAGLIFLOZIN\METFORMIN HYDROCHLORIDE:Cardiac disorder:0.42), (EPHEDRINE HYDROCHLORIDE\GUAIFENESIN:Cardiac disorder:0.39), (EPIRUBICIN:Cardiac disorder:1.08), (ERIBULIN:Cardiac disorder:0), (ESTRADIOL BENZOATE:Cardiac disorder:0.05), (ESTROGENS\METHYLTESTOSTERONE:Cardiac disorder:0.13), (ETHINYL ESTRADIOL\NORGESTREL:Cardiac disorder:0.79), (ETOPOSIDE PHOSPHATE:Cardiac disorder:0.23), (FERRIC HYDROXIDE:Cardiac disorder:0.75), (FERRIC POLYSACCHARIDE COMPLEX:Cardiac disorder:0.66), (FESOTERODINE:Cardiac disorder:0.27), (FEXOFENADINE HYDROCHLORIDE\PSEUDOEPHEDRINE HYDROCHLORIDE:Cardiac disorder:0.24), (FLUNARIZINE HYDROCHLORIDE:Cardiac disorder:0.94), (FLUOCINOLONE ACETONIDE:Cardiac disorder:1.06), (FLUOXETINE HYDROCHLORIDE\OLANZAPINE:Cardiac disorder:0.17), (FLUPHENAZINE:Cardiac disorder:0.05), (FLUTICASONE PROPIONATE\FORMOTEROL FUMARATE:Cardiac disorder:0.09), (FORMOTEROL FUMARATE\MOMETASONE FUROATE:Cardiac disorder:1.67), (FOSFOMYCIN:Cardiac disorder:0.28), (FROVATRIPTAN SUCCINATE:Cardiac disorder:1), (GIMERACIL\OTERACIL\TEGAFUR:Cardiac disorder:0.25), (GOLD:Cardiac disorder:0.1), (GREEN TEA LEAF:Cardiac disorder:0.15), (HALCINONIDE:Cardiac disorder:0.21), (HALOPERIDOL DECANOATE:Cardiac disorder:0.35), (HEPARIN CALCIUM:Cardiac disorder:1.42), (HERBALS\MINERALS\VITAMINS:Cardiac disorder:0.22), (HOMATROPINE METHYLBROMIDE\HYDROCODONE BITARTRATE:Cardiac disorder:0.14), (HUMAN C1-ESTERASE INHIBITOR:Cardiac disorder:0.34), (HUMAN HEPATITIS B VIRUS IMMUNE GLOBULIN:Cardiac disorder:0.27), (HYDROCHLOROTHIAZIDE\METOPROLOL TARTRATE:Cardiac disorder:0.18), (HYDROQUINONE:Cardiac disorder:0.15), (HYDROXYPROPYL CELLULOSE:Cardiac disorder:0.56), (HYOSCYAMINE SULFATE\METHENAMINE\METHYLENE BLUE\PHENYL SALICYLATE\SODIUM PHOSPHATE, MONOBASIC, MONOHYDRATE:Cardiac disorder:0.9), (IBRITUMOMAB TIUXETAN:Cardiac disorder:0.52), (IMIGLUCERASE:Cardiac disorder:0.95), (IMIPENEM:Cardiac disorder:0.53), (IMMUNE GLOBULIN NOS:Cardiac disorder:0.46), (INFLIXIMAB-DYYB:Cardiac disorder:0.29), (INSULIN ASPART\INSULIN DEGLUDEC:Cardiac disorder:0.16), (INSULIN PORK\INSULIN PURIFIED PORK:Cardiac disorder:1.32), (INTERFERON:Cardiac disorder:1.21), (INTERFERON ALFA-2A:Cardiac disorder:1.49), (INTERFERON ALFA-2B:Cardiac disorder:0.57), (INTERFERON GAMMA-1B:Cardiac disorder:0.76), (IRINOTECAN HYDROCHLORIDE:Cardiac disorder:0.64), (ITRACONAZOLE\SODIUM CHLORIDE:Cardiac disorder:0.4), (LANREOTIDE:Cardiac disorder:1.02), (LATANOPROST\TIMOLOL:Cardiac disorder:0.71), (LENVATINIB:Cardiac disorder:0.38), (LEVOBUNOLOL HYDROCHLORIDE:Cardiac disorder:0.34), (LEVOLEUCOVORIN CALCIUM:Cardiac disorder:0.01), (LEVOMEFOLIC ACID\METHYLCOBALAMIN\TURMERIC:Cardiac disorder:0.04), (LEVOSIMENDAN:Cardiac disorder:0.17), (LEVOTHYROXINE SODIUM\POTASSIUM IODIDE:Cardiac disorder:0.38), (LOMITAPIDE MESYLATE:Cardiac disorder:1.93), (LOMUSTINE:Cardiac disorder:0.33), (LUTETIUM LU-177:Cardiac disorder:0.76), (MAGNESIUM ASPARTATE HYDROCHLORIDE:Cardiac disorder:0.49), (MECLIZINE MONOHYDROCHLORIDE\NIACIN:Cardiac disorder:0.14), (MELPERONE:Cardiac disorder:0.26), (MENTHOL\ZINC OXIDE:Cardiac disorder:0.69), (MEPERIDINE:Cardiac disorder:1.58), (MESNA:Cardiac disorder:0.64), (METHENOLONE ENANTHATE:Cardiac disorder:0.37), (METRELEPTIN:Cardiac disorder:0.81), (MIGLUSTAT:Cardiac disorder:0.2), (MOLSIDOMINE:Cardiac disorder:0.26), (NAFTIFINE HYDROCHLORIDE:Cardiac disorder:0.28), (NALOXONE:Cardiac disorder:0.38), (NALOXONE HYDROCHLORIDE:Cardiac disorder:0.13), (NALTREXONE HYDROCHLORIDE:Cardiac disorder:0.15), (NAPROXEN SODIUM\SUMATRIPTAN SUCCINATE:Cardiac disorder:0.62), (NEVIRAPINE:Cardiac disorder:0.13), (NIRAPARIB:Cardiac disorder:1.19), (NOREPINEPHRINE:Cardiac disorder:0.92), (OLOPATADINE:Cardiac disorder:0.89), (OSELTAMIVIR:Cardiac disorder:1.13), (PANITUMUMAB:Cardiac disorder:0.15), (PAPAYA:Cardiac disorder:0.34), (PARATHYROID HORMONE:Cardiac disorder:0.78), (PEGZEREPOETIN ALFA:Cardiac disorder:0.1), (PENICILLAMINE:Cardiac disorder:0.08), (PENTAMIDINE ISETHIONATE:Cardiac disorder:0.37), (PENTOSTATIN:Cardiac disorder:0.64), (PEPSIN:Cardiac disorder:0.11), (PHENYLEPHRINE HYDROCHLORIDE\WITCH HAZEL:Cardiac disorder:0.56), (PHLOROGLUCINOL1,3,5-TRIMETHOXYBENZENE:Cardiac disorder:0.15), (PIMOBENDAN:Cardiac disorder:0.32), (PINAVERIUM BROMIDE:Cardiac disorder:0.31), (PIRITRAMIDE:Cardiac disorder:0.04), (POLYETHYLENE GLYCOL 400:Cardiac disorder:0.71), (POLYVINYL ALCOHOL:Cardiac disorder:0.33), (POTASSIUM PHOSPHATE, MONOBASIC:Cardiac disorder:0.42), (POTASSIUM PHOSPHATE, MONOBASIC\SODIUM PHOSPHATE, DIBASIC, ANHYDROUS\SODIUM PHOSPHATE, MONOBASIC, MONOHYDRATE:Cardiac disorder:0.03), (POVIDONE:Cardiac disorder:0.36), (PRAMOXINE HYDROCHLORIDE:Cardiac disorder:0.51), (PREDNISOLONE SODIUM PHOSPHATE:Cardiac disorder:0.93), (PREDNISOLONE\PREDNISOLONE ACETATE\PREDNISOLONE SODIUM SUCCINATE:Cardiac disorder:1.54), (PREGNENOLONE:Cardiac disorder:0.43), (PROTRIPTYLINE HYDROCHLORIDE:Cardiac disorder:1.69), (PYRAZINAMIDE:Cardiac disorder:0.79), (QUINACRINE:Cardiac disorder:0.71), (QUINAGOLIDE HYDROCHLORIDE:Cardiac disorder:0.08), (QUINIDINE GLUCONATE:Cardiac disorder:0.33), (RADIUM RA-223 DICHLORIDE:Cardiac disorder:0.12), (REGORAFENIB:Cardiac disorder:0.91), (RILPIVIRINE HYDROCHLORIDE:Cardiac disorder:0.44), (ROSIGLITAZONE:Cardiac disorder:0.53), (RUPATADINE FUMARATE:Cardiac disorder:0.02), (SODIUM BICARBONATE\SODIUM CHLORIDE:Cardiac disorder:0.19), (SODIUM CITRATE:Cardiac disorder:0.6), (SULFACETAMIDE SODIUM\SULFUR:Cardiac disorder:0.18), (TEDUGLUTIDE:Cardiac disorder:0.07), (TEDUGLUTIDE\WATER:Cardiac disorder:0.92), (TELOTRISTAT ETHYL:Cardiac disorder:0.67), (TENOFOVIR:Cardiac disorder:0.05), (TESTOSTERONE UNDECANOATE:Cardiac disorder:1.67), (TIAGABINE HYDROCHLORIDE:Cardiac disorder:0.09), (TINZAPARIN:Cardiac disorder:1.23), (TIOPRONIN:Cardiac disorder:0.04), (TREPROSTINIL DIOLAMINE:Cardiac disorder:0.71), (TRIMIPRAMINE MALEATE:Cardiac disorder:1.44), (UROKINASE:Cardiac disorder:0.05), (VALRUBICIN:Cardiac disorder:0.04), (VINORELBINE TARTRATE:Cardiac disorder:0.93), (VINORELBINE\VINORELBINE TARTRATE:Cardiac disorder:0.02), (VITAMIN A PALMITATE:Cardiac disorder:0.2), (XIPAMIDE:Cardiac disorder:0.75), (ZEAXANTHIN:Cardiac disorder:0.51), (ZICONOTIDE ACETATE:Cardiac disorder:0.66), (.ALPHA.-TOCOPHEROL\RETINOL:Cardiac failure:0.8), (.ALPHA.1-PROTEINASE INHIBITOR HUMAN:Cardiac failure:0.53), (ACETAMINOPHEN\APRONALIDE\CAFFEINE\PROPYPHENAZONE:Cardiac failure:0.66), (ACETAMINOPHEN\BUTALBITAL\CAFFEINE:Cardiac failure:1.35), (ACETAMINOPHEN\PROPOXYPHENE HYDROCHLORIDE:Cardiac failure:0.48), (ACETAMINOPHEN\PROPOXYPHENE NAPSYLATE:Cardiac failure:0.01), (ACITRETIN:Cardiac failure:0.49), (ADENINE:Cardiac failure:1.14), (ADO-TRASTUZUMAB EMTANSINE:Cardiac failure:1.28), (ADUCANUMAB:Cardiac failure:0.11), (ALCOHOL:Cardiac failure:0.83), (ALISKIREN HEMIFUMARATE:Cardiac failure:0.83), (ALOGLIPTIN:Cardiac failure:0.55), (ALOGLIPTIN BENZOATE\PIOGLITAZONE HYDROCHLORIDE:Cardiac failure:0.18), (ALUMINUM SILICATE:Cardiac failure:0.67), (AMOXAPINE:Cardiac failure:1.3), (AMOXICILLIN\CLARITHROMYCIN\ESOMEPRAZOLE:Cardiac failure:0.54), (AMPHETAMINE ASPARTATE\AMPHETAMINE SULFATE\DEXTROAMPHETAMINE SACCHARATE\DEXTROAMPHETAMINE SULFATE:Cardiac failure:0.88), (AMPICILLIN SODIUM:Cardiac failure:0.82), (ANAGLIPTIN:Cardiac failure:1.62), (ANTIHISTAMINES NOS:Cardiac failure:0.17),(ARMODAFINIL:Cardiac failure:0.53), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Cardiac failure:0.73), (ASCORBIC ACID\FERROUS SULFATE:Cardiac failure:0.06), (ASPIRIN\DIHYDROXYALUMINUM AMINOACETATE\MAGNESIUM CARBONATE:Cardiac failure:0.06), (AVOBENZONE\HOMOSALATE\OCTISALATE:Cardiac failure:0.11), (AZELASTINE HYDROCHLORIDE:Cardiac failure:0.22), (BACITRACIN:Cardiac failure:0.81), (BARNIDIPINE:Cardiac failure:0.09), (BECLOMETHASONE:Cardiac failure:0.64), (BENPROPERINE PHOSPHATE:Cardiac failure:0.05), (BENZTROPINE MESYLATE:Cardiac failure:0.16), (BETAMETHASONE BUTYRATE PROPIONATE:Cardiac failure:0.99), (BETAMETHASONE DIPROPIONATE\CALCIPOTRIENE\CALCIPOTRIENE HYDRATE:Cardiac failure:0.18), (BETAMETHASONE SODIUM PHOSPHATE\NEOMYCIN SULFATE:Cardiac failure:0.71), (BETAMETHASONE\CALCIPOTRIENE:Cardiac failure:0.51), (BETHANECHOL CHLORIDE:Cardiac failure:0.73), (BICARBONATE ION:Cardiac failure:0.73), (BIXALOMER:Cardiac failure:0.7), (BLESELUMAB:Cardiac failure:0.14), (BLINATUMOMAB:Cardiac failure:0.33), (BLONANSERIN:Cardiac failure:0.14), (BOTULINUM TOXIN TYPE A:Cardiac failure:0.16), (BROMPERIDOL:Cardiac failure:0.04), (BUFORMIN:Cardiac failure:0.42), (CABAZITAXEL:Cardiac failure:0.73), (CABOZANTINIB:Cardiac failure:0.19), (CALCITONIN:Cardiac failure:1.05), (CALCITONIN SALMON:Cardiac failure:0.8), (CALCIUM CARBONATE\ERGOCALCIFEROL\RETINOL:Cardiac failure:0.56), (CALCIUM\MAGNESIUM:Cardiac failure:0.39), (CALCIUM\MAGNESIUM\ZINC:Cardiac failure:0.08), (CAMPHOR\CAPSICUM\METHYL SALICYLATE:Cardiac failure:0.04), (CARBIDOPA\ENTACAPONE\LEVODOPA:Cardiac failure:0.25), (CARMUSTINE:Cardiac failure:0.01), (CARTEOLOL:Cardiac failure:0.09), (CEFOTAXIME\CEFOTAXIME SODIUM:Cardiac failure:0.48), (CEFTERAM PIVOXIL:Cardiac failure:0.07), (CELIPROLOL:Cardiac failure:0.64), (CELLULASE\DIASTASE\LIPASE:Cardiac failure:0.94), (CHLORAMPHENICOL:Cardiac failure:0.26), (CHLORDIAZEPOXIDE HYDROCHLORIDE\CLIDINIUM BROMIDE:Cardiac failure:0.23), (CHONDROITIN SULFATE (CHICKEN):Cardiac failure:0.55), (CIPROFLOXACIN\DEXAMETHASONE:Cardiac failure:0.1), (CLOPERASTINE:Cardiac failure:0.54), (CLOXACILLIN SODIUM:Cardiac failure:0.5), (CODEINE PHOSPHATE\GUAIFENESIN:Cardiac failure:0.36), (COLESTILAN CHLORIDE:Cardiac failure:0.18), (COLISTIMETHATE SODIUM:Cardiac failure:0.2), (COLISTIN:Cardiac failure:0.11), (CORTICOTROPIN:Cardiac failure:0.7), (CORTISONE\HYDROCORTISONE:Cardiac failure:1.87), (CYANAMIDE:Cardiac failure:0.09), (CYPROHEPTADINE HYDROCHLORIDE:Cardiac failure:0.18), (DACLIZUMAB:Cardiac failure:1.22), (DALFAMPRIDINE:Cardiac failure:0.49), (DANAZOL:Cardiac failure:1.36), (DAPAGLIFLOZIN PROPANEDIOL:Cardiac failure:1.18), (DASABUVIR\OMBITASVIR\PARITAPREVIR\RITONAVIR:Cardiac failure:0.31), (DEQUALINIUM CHLORIDE:Cardiac failure:0.23), (DESMOPRESSIN:Cardiac failure:0.37), (DEXTRAN:Cardiac failure:0.12), (DEXTROAMPHETAMINE SULFATE:Cardiac failure:0.27), (DIMEMORFAN PHOSPHATE:Cardiac failure:0.42), (DOLASETRON MESYLATE:Cardiac failure:0.37), (DOXEPIN HYDROCHLORIDE:Cardiac failure:0.63), (DOXYCYCLINE HYCLATE:Cardiac failure:0.17), (DUTASTERIDE\TAMSULOSIN HYDROCHLORIDE:Cardiac failure:1.37), (ECABET SODIUM:Cardiac failure:0.94), (EDARAVONE:Cardiac failure:1.13), (ELETRIPTAN HYDROBROMIDE:Cardiac failure:1.15), (ELTROMBOPAG OLAMINE:Cardiac failure:0.17), (EMPAGLIFLOZIN\LINAGLIPTIN:Cardiac failure:1.16), (ENTACAPONE:Cardiac failure:0.05), (EPALRESTAT:Cardiac failure:1.35), (EPHEDRA SINICA STEM\HERBALS:Cardiac failure:0.03), (ERIBULIN:Cardiac failure:0.32), (ESTROGENS, CONJUGATED:Cardiac failure:0.92), (ESTROGENS, CONJUGATED\MEDROXYPROGESTERONE ACETATE:Cardiac failure:0.09), (ETHAMBUTOL HYDROCHLORIDE:Cardiac failure:0.96), (ETRETINATE:Cardiac failure:1.95), (FELODIPINE\METOPROLOL SUCCINATE:Cardiac failure:0.11), (FISH OIL\OMEGA-3 FATTY ACIDS:Cardiac failure:0.33), (FLUNARIZINE HYDROCHLORIDE:Cardiac failure:0.05), (FLUOCINOLONE ACETONIDE:Cardiac failure:0.3), (FLUPHENAZINE:Cardiac failure:0.06), (FLURAZEPAM\FLURAZEPAM HYDROCHLORIDE:Cardiac failure:0.09), (FLUVOXAMINE:Cardiac failure:0.19), (FORMOTEROL FUMARATE\MOMETASONE FUROATE:Cardiac failure:0.73), (FOSFLUCONAZOLE:Cardiac failure:0.38), (FOSFOMYCIN:Cardiac failure:0.81), (FOSFOMYCIN CALCIUM:Cardiac failure:0.42), (FOSINOPRIL SODIUM:Cardiac failure:0.08), (FOSPHENYTOIN\FOSPHENYTOIN SODIUM:Cardiac failure:0.62), (FURSULTIAMINE:Cardiac failure:1.43), (GABEXATE MESYLATE:Cardiac failure:0.36), (GLIMEPIRIDE\METFORMIN:Cardiac failure:0.03), (GLIMEPIRIDE\ROSIGLITAZONE MALEATE:Cardiac failure:0.83), (GUANFACINE HYDROCHLORIDE:Cardiac failure:0.07), (HYDROCHLOROTHIAZIDE\LOSARTAN:Cardiac failure:0.11), (HYDROCODONE BITARTRATE\IBUPROFEN:Cardiac failure:0.08), (HYDROCORTISONE ACETATE:Cardiac failure:0.84), (HYOSCYAMINE:Cardiac failure:0.83), (IBUDILAST:Cardiac failure:0.39), (IFENPRODIL TARTRATE:Cardiac failure:0.47), (IGURATIMOD:Cardiac failure:1.27), (IMIGLUCERASE:Cardiac failure:0.61), (IMIPRAMINE HYDROCHLORIDE:Cardiac failure:0.59), (IMIQUIMOD:Cardiac failure:0.15), (IMMUNE GLOBULIN NOS:Cardiac failure:0.5), (INFLIXIMAB-DYYB:Cardiac failure:0.31), (INTERFERON:Cardiac failure:0.6), (INTERFERON ALFA-2B:Cardiac failure:0.51), (IPRAGLIFLOZIN:Cardiac failure:0.13), (IRINOTECAN HYDROCHLORIDE:Cardiac failure:1.27), (ISONIAZID\PYRAZINAMIDE\RIFAMPIN:Cardiac failure:0.59), (LANREOTIDE:Cardiac failure:0.19), (LAPATINIB DITOSYLATE:Cardiac failure:0.5), (LENVATINIB:Cardiac failure:0.38), (LEVOCABASTINE HYDROCHLORIDE:Cardiac failure:1.01), (LICORICE ROOT EXTRACT\RHUBARB:Cardiac failure:0.2), (LIOTHYRONINE:Cardiac failure:0.18), (LISDEXAMFETAMINE DIMESYLATE:Cardiac failure:0.31), (LOBUCAVIR:Cardiac failure:0.06), (LOPRAZOLAM:Cardiac failure:0.11), (LURASIDONE HYDROCHLORIDE:Cardiac failure:0.01), (LYSOZYME HYDROCHLORIDE:Cardiac failure:0.94), (MAGALDRATE:Cardiac failure:0.24), (MAGNESIUM PIDOLATE:Cardiac failure:0.1), (MEPENZOLATE BROMIDE:Cardiac failure:0.49), (MEPOLIZUMAB:Cardiac failure:0.53), (MESNA:Cardiac failure:0.91), (METFORMIN HYDROCHLORIDE\ROSIGLITAZONE MALEATE:Cardiac failure:1.22), (METFORMIN HYDROCHLORIDE\SAXAGLIPTIN:Cardiac failure:0.58), (METHENAMINE HIPPURATE:Cardiac failure:1.2), (METHENOLONE ACETATE:Cardiac failure:0.06), (METHYLPREDNISOLONE ACETATE:Cardiac failure:1.56), (MIGLUSTAT:Cardiac failure:0.46), (MOEXIPRIL HYDROCHLORIDE:Cardiac failure:0.54), (MORPHINE SULFATE\NALTREXONE HYDROCHLORIDE:Cardiac failure:0.46), (NABUMETONE:Cardiac failure:1.04), (NALFURAFINE HYDROCHLORIDE:Cardiac failure:0.43), (NALOXONE:Cardiac failure:0.36), (NORTRIPTYLINE HYDROCHLORIDE:Cardiac failure:0.17), (OLAPARIB:Cardiac failure:0.12), (ORPHENADRINE:Cardiac failure:0.45), (OXYBUTYNIN\OXYBUTYNIN CHLORIDE:Cardiac failure:0.19), (OXYMETAZOLINE:Cardiac failure:0.3), (PALIPERIDONE PALMITATE:Cardiac failure:0.09), (PANTOPRAZOLE MAGNESIUM:Cardiac failure:2.22), (PASIREOTIDE:Cardiac failure:0.7), (PAZUFLOXACIN MESILATE:Cardiac failure:0.2), (PEMETREXED DISODIUM:Cardiac failure:1.97), (PENTOSAN POLYSULFATE SODIUM:Cardiac failure:0.26), (PENTOSTATIN:Cardiac failure:0.46), (PEROSPIRONE HYDROCHLORIDE:Cardiac failure:0.09), (PIRENZEPINE:Cardiac failure:0.27), (PORFIMER SODIUM:Cardiac failure:0.28), (POTASSIUM PHOSPHATE, MONOBASIC:Cardiac failure:0.13), (POVIDONE:Cardiac failure:0.18), (PREDNISOLONE SODIUM PHOSPHATE:Cardiac failure:0.42), (PREDNISOLONE VALERATE ACETATE:Cardiac failure:0.25), (PROCATEROL HYDROCHLORIDE:Cardiac failure:0.58), (PYGEUM:Cardiac failure:0.51), (PYRIDOXAL PHOSPHATE:Cardiac failure:0.12), (PYRIDOXINE:Cardiac failure:0.68), (QUAZEPAM:Cardiac failure:0.32), (QUINACRINE:Cardiac failure:0.16), (QUINAPRIL HYDROCHLORIDE:Cardiac failure:0.16), (RADIATION THERAPY:Cardiac failure:0.77), (RAMUCIRUMAB:Cardiac failure:0.99), (RETINOL:Cardiac failure:0.56), (RIFABUTIN:Cardiac failure:0.06), (RILPIVIRINE HYDROCHLORIDE:Cardiac failure:0.41), (RISEDRONIC ACID:Cardiac failure:0.15), (RIZATRIPTAN BENZOATE:Cardiac failure:0.65), (ROXATIDINE ACETATE HYDROCHLORIDE:Cardiac failure:0.05), (SELEGILINE:Cardiac failure:0.04), (SENNOSIDE B:Cardiac failure:0.36), (SERRAPEPTASE:Cardiac failure:0.22), (SIPULEUCEL-T:Cardiac failure:0.14), (SODIUM FLUORIDE:Cardiac failure:1.2), (SODIUM GUALENATE MONOHYDRATE:Cardiac failure:0.2), (SOFALCONE:Cardiac failure:0.36), (SORBITOL:Cardiac failure:1.16), (SUCCINYLCHOLINE CHLORIDE:Cardiac failure:0.23), (SUMATRIPTAN:Cardiac failure:0.68), (SUMATRIPTAN\SUMATRIPTAN SUCCINATE:Cardiac failure:0.68), (SUPLATAST TOSILATE:Cardiac failure:1.33), (TAFLUPROST:Cardiac failure:0.39), (TANDOSPIRONE:Cardiac failure:0.21), (TEDUGLUTIDE\WATER:Cardiac failure:0.08), (TEGAFUR\URACIL:Cardiac failure:0.14), (TELOTRISTAT ETHYL:Cardiac failure:0.08), (TEMSIROLIMUS:Cardiac failure:0.5), (TENECTEPLASE:Cardiac failure:0.02), (TERIFLUNOMIDE:Cardiac failure:0.1), (TESTOSTERONE ENANTHATE:Cardiac failure:0.01), (TESTOSTERONE UNDECANOATE:Cardiac failure:1.08), (TETRACYCLINE:Cardiac failure:1.09), (TIGECYCLINE:Cardiac failure:0.82), (TIMOLOL\TRAVOPROST:Cardiac failure:0.47), (TIPIRACIL HYDROCHLORIDE\TRIFLURIDINE:Cardiac failure:0.13), (TRIHEXYPHENIDYL:Cardiac failure:0.83), (TRIMEPRAZINE:Cardiac failure:0.09), (TRIPTORELIN PAMOATE:Cardiac failure:0.04), (UROKINASE:Cardiac failure:1.1), (VARDENAFIL HYDROCHLORIDE TRIHYDRATE:Cardiac failure:0.46), (VILAZODONE HYDROCHLORIDE:Cardiac failure:0.07), (VONOPRAZAN:Cardiac failure:0.56), (ZAFIRLUKAST:Cardiac failure:0.43), (ZALTOPROFEN:Cardiac failure:0.46), (ZIV-AFLIBERCEPT:Cardiac failure:0), (ZOLMITRIPTAN:Cardiac failure:0.09), (ACEBUTOLOL HYDROCHLORIDE:Cardiac failure chronic:0.04), (ACETAMINOPHEN\APRONALIDE\CAFFEINE\PROPYPHENAZONE:Cardia c failure chronic:0.06), (ACLIDINIUM BROMIDE:Cardiac failure chronic:0.03), (ADENINE:Cardiac failure chronic:0.13), (AGALSIDASE BETA:Cardiac failure chronic:0.48), (ALBUMIN HUMAN:Cardiac failure chronic:0.53), (AMBROXOL\CLENBUTEROL:Cardiac failure chronic:0.59), (AMLODIPINE BESYLATE\ATORVASTATIN CALCIUM:Cardiac failure chronic:0.29), (AMLODIPINE\VALSARTAN:Cardiac failure chronic:0.19), (AMOXAPINE:Cardiac failure chronic:0.02), (ANAGRELIDE HYDROCHLORIDE:Cardiac failure chronic:0.12), (ASPIRIN\MAGNESIUM OXIDE:Cardiac failure chronic:0.03), (AXITINIB:Cardiac failure chronic:0.03), (AZITHROMYCIN DIHYDRATE:Cardiac failure chronic:0.18), (BENZONATATE:Cardiac failure chronic:0.19), (BEPRIDIL:Cardiac failure chronic:2.17), (BETAMETHASONE DIPROPIONATE:Cardiac failure chronic:0.05), (BETAMETHASONE SODIUM PHOSPHATE\NEOMYCIN SULFATE:Cardiac failure chronic:0.34), (BISACODYL:Cardiac failure chronic:0.73), (BIXALOMER:Cardiac failure chronic:0.23), (BROMAZEPAM:Cardiac failure chronic:0.68), (BUDESONIDE\FORMOTEROL FUMARATE:Cardiac failure chronic:0.91), (CALCIUM CARBONATE\CHOLECALCIFEROL:Cardiac failure chronic:1.23), (CALCIUM CHLORIDE\DEXTROSE\POTASSIUM CHLORIDE\SODIUM ACETATE\SODIUM CHLORIDE:Cardiac failure chronic:0.11), (CANAGLIFLOZIN\METFORMIN HYDROCHLORIDE:Cardiac failure chronic:0.23), (CARFILZOMIB:Cardiac failure chronic:0.63), (CEFCAPENE PIVOXIL HYDROCHLORIDE HYDRATE:Cardiac failure chronic:0.44), (CERTOLIZUMAB PEGOL:Cardiac failure chronic:1.05), (CHLORTHALIDONE:Cardiac failure chronic:0.34), (CICLESONIDE:Cardiac failure chronic:0.47), (CIMETIDINE:Cardiac failure chronic:0.1), (CLINDAMYCIN:Cardiac failure chronic:0.05), (CLINDAMYCIN\CLINDAMYCIN PHOSPHATE:Cardiac failure chronic:0.9), (CLOZAPINE:Cardiac failure chronic:0.57), (CORTISONE\HYDROCORTISONE:Cardiac failure chronic:0.73), (DANAZOL:Cardiac failure chronic:0.15), (DAPAGLIFLOZIN PROPANEDIOL:Cardiac failure chronic:0.39), (DASATINIB:Cardiac failure chronic:0.92), (DESFLURANE:Cardiac failure chronic:0.08), (DESLORATADINE:Cardiac failure chronic:0.58), (DEVICE:Cardiac failure chronic:0.61), (DIMENHYDRINATE:Cardiac failure chronic:0.37), (DISOPYRAMIDE:Cardiac failure chronic:0.19), (DIVALPROEX SODIUM:Cardiac failure chronic:0.27), (DOCUSATE CALCIUM:Cardiac failure chronic:0.26), (DRONABINOL:Cardiac failure chronic:0.15), (ECABET SODIUM:Cardiac failure chronic:0.23), (ECONAZOLE:Cardiac failure chronic:0.21), (ELETRIPTAN HYDROBROMIDE:Cardiac failure chronic:0.19), (EMPAGLIFLOZIN\LINAGLIPTIN:Cardiac failure chronic:0.05), (ERYTHROMYCIN:Cardiac failure chronic:0.19), (ETRETINATE:Cardiac failure chronic:0.44), (EVEROLIMUS:Cardiac failure chronic:1.55), (FAROPENEM SODIUM:Cardiac failure chronic:0.04), (FERRIC HYDROXIDE:Cardiac failure chronic:1.61), (FERRIC OXYHYDROXIDE:Cardiac failure chronic:0.44), (FLUTICASONE:Cardiac failure chronic:0.67), (FORMOTEROL FUMARATE DIHYDRATE\MOMETASONE FUROATE:Cardiac failure chronic:0.25), (FORMOTEROL FUMARATE\MOMETASONE FUROATE:Cardiac failure chronic:0.27), (FURSULTIAMINE:Cardiac failure chronic:0.96), (GEMFIBROZIL:Cardiac failure chronic:0.06), (GLIMEPIRIDE\ROSIGLITAZONE MALEATE:Cardiac failure chronic:0.6), (GUANABENZ ACETATE:Cardiac failure chronic:1.27), (HYDROCHLOROTHIAZIDE\IRBESARTAN:Cardiac failure chronic:1.01), (HYDROCHLOROTHIAZIDE\LISINOPRIL:Cardiac failure chronic:1.01), (HYDROCHLOROTHIAZIDE\METOPROLOL SUCCINATE:Cardiac failure chronic:0.04), (HYDROMORPHONE HYDROCHLORIDE:Cardiac failure chronic:0.59), (HYDROXYZINE PAMOATE:Cardiac failure chronic:0.33), (IBRUTINIB:Cardiac failure chronic:1.05), (IGURATIMOD:Cardiac failure chronic:0.21), (ILOPROST:Cardiac failure chronic:1.73), (IPRAGLIFLOZIN L-PROLINE:Cardiac failure chronic:0.44), (IRON SUCROSE:Cardiac failure chronic:0.66), (LANDIOLOL HYDROCHLORIDE:Cardiac failure chronic:0.14), (LERCANIDIPINE HYDROCHLORIDE:Cardiac failure chronic:1.34), (LIDOCAINE\PRILOCAINE:Cardiac failure chronic:0.08), (LITHIUM CARBONATE:Cardiac failure chronic:0.99), (MEPOLIZUMAB:Cardiac failure chronic:0.33), (METFORMIN HYDROCHLORIDE\ROSIGLITAZONE MALEATE:Cardiac failure chronic:1.08), (METHADONE HYDROCHLORIDE:Cardiac failure chronic:0.56), (METRONIDAZOLE\METRONIDAZOLE HYDROCHLORIDE:Cardiac failure chronic:0.02), (MINOCYCLINE\MINOCYCLINE HYDROCHLORIDE:Cardiac failure chronic:0.42), (NALOXONE HYDROCHLORIDE\TILIDINE HYDROCHLORIDE:Cardiac failure chronic:0.39), (NAPROXEN SODIUM:Cardiac failure chronic:0.35), (NATEGLINIDE:Cardiac failure chronic:0.75), (NILOTINIB:Cardiac failure chronic:1.77), (NINTEDANIB:Cardiac failure chronic:0.55), (NOREPINEPHRINE BITARTRATE:Cardiac failure chronic:0.69), (NORTRIPTYLINE:Cardiac failure chronic:0.09), (OMEGA-3-CARBOXYLIC ACIDS:Cardiac failure chronic:0.13), (OMEPRAZOLE SODIUM:Cardiac failure chronic:0.4), (OXCARBAZEPINE:Cardiac failure chronic:0.53), (PANCRELIPASE AMYLASE\PANCRELIPASE LIPASE\PANCRELIPASE PROTEASE:Cardiac failure chronic:0.07), (PANTOPRAZOLE MAGNESIUM:Cardiac failure chronic:1.07), (PATIROMER:Cardiac failure chronic:0.23), (PAZOPANIB HYDROCHLORIDE:Cardiac failure chronic:0.32), (PEGZEREPOETIN ALFA:Cardiac failure chronic:0.78), (PEMETREXED DISODIUM:Cardiac failure chronic:0.09), (PENICILLIN:Cardiac failure chronic:0.28), (PHENOBARBITAL:Cardiac failure chronic:0.09), (PHYTONADIONE:Cardiac failure chronic:0.15), (POLYETHYLENE GLYCOL 4000:Cardiac failure chronic:0.23), (PONATINIB:Cardiac failure chronic:0.24), (PREDNISOLONE ACETATE:Cardiac failure chronic:0.37), (PROMETHAZINE\PROMETHAZINE HYDROCHLORIDE:Cardiac failure chronic:0.65), (PROPAFENONE HYDROCHLORIDE:Cardiac failure chronic:0.77), (PROPIVERINE:Cardiac failure chronic:0.2), (RIVASTIGMINE TARTRATE:Cardiac failure chronic:0.11), (ROPINIROLE HYDROCHLORIDE:Cardiac failure chronic:0.37), (SALMETEROL:Cardiac failure chronic:0.15), (SALMETEROL XINAFOATE:Cardiac failure chronic:0.14), (SCOPOLAMINE:Cardiac failure chronic:0.32), (SORBITOL:Cardiac failure chronic:0.1), (SULFAMETHOXAZOLE:Cardiac failure chronic:0.2), (SUNITINIB MALATE:Cardiac failure chronic:0.03), (SUPLATAST TOSILATE:Cardiac failure chronic:0.65), (TAFAMIDIS MEGLUMINE:Cardiac failure chronic:0.47), (TERAZOSIN HYDROCHLORIDE:Cardiac failure chronic:0.16), (TERBINAFINE HYDROCHLORIDE:Cardiac failure chronic:0), (TERBUTALINE:Cardiac failure chronic:0.16), (TESTOSTERONE UNDECANOATE:Cardiac failure chronic:0.02), (THIOCTIC ACID:Cardiac failure chronic:0.34), (TICLOPIDINE:Cardiac failure chronic:0.46), (TRIAMTERENE:Cardiac failure chronic:0.03), (VARENICLINE TARTRATE:Cardiac failure chronic:0.18), (.ALPHA.1-PROTEINASE INHIBITOR HUMAN:Cardiac failure congestive:1.2), (ABACAVIR SULFATE\DOLUTEGRAVIR SODIUM\LAMIVUDINE:Cardiac failure congestive:0.36), (ACETAMINOPHEN\CAFFEINE:Cardiac failure congestive:0.3), (ACETAMINOPHEN\PROPOXYPHENE HYDROCHLORIDE:Cardiac failure congestive:0.27), (ACETAMINOPHEN\PROPOXYPHENE NAPSYLATE:Cardiac failure congestive:0.04), (ACITRETIN:Cardiac failure congestive:0.11), (ADO-TRASTUZUMAB EMTANSINE:Cardiac failure congestive:1.03), (ALBIGLUTIDE:Cardiac failure congestive:0.8), (ALEMTUZUMAB:Cardiac failure congestive:0.19), (AMANTADINE:Cardiac failure congestive:0.65), (AMINO ACIDS\DEXTROSE\ELECTROLYTES NOS:Cardiac failure congestive:0.58), (AMISULPRIDE:Cardiac failure congestive:0.76), (AMLODIPINE BESYLATE\HYDROCHLOROTHIAZIDE\OLMESARTAN MEDOXOMIL:Cardiac failure congestive:0.35), (AMLODIPINE BESYLATE\HYDROCHLOROTHIAZIDE\VALSARTAN:Cardiac failure congestive:0.62), (AMLODIPINE\PERINDOPRIL:Cardiac failure congestive:0.11), (ANTIHISTAMINES NOS:Cardiac failure congestive:0.01), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Cardiac failure congestive:0.24), (ASPIRIN\CLOPIDOGREL:Cardiac failure congestive:0.11), (ASPIRIN\DIHYDROXYALUMINUM AMINOACETATE ANHYDROUS\MAGNESIUM CARBONATE:Cardiac failure congestive:0.03), (ASPIRIN\MAGNESIUM OXIDE:Cardiac failure congestive:0.12), (BACITRACIN:Cardiac failure congestive:0.41), (BERAPROST:Cardiac failure congestive:0.57), (BETAMETHASONE DIPROPIONATE\CALCIPOTRIENE\CALCIPOTRIENE HYDRATE:Cardiac failure congestive:0.67), (BETAMETHASONE DIPROPIONATE\CLOTRIMAZOLE:Cardiac failure congestive:0.18), (BETHANECHOL CHLORIDE:Cardiac failure congestive:0.26), (BLINATUMOMAB:Cardiac failure congestive:0.16), (BUDESONIDE\FORMOTEROL FUMARATE:Cardiac failure congestive:1.11), (CABAZITAXEL:Cardiac failure congestive:0.1), (CALCITONIN:Cardiac failure congestive:0.59), (CALCIUM\MAGNESIUM:Cardiac failure congestive:0.57), (CALCIUM\MAGNESIUM\ZINC:Cardiac failure congestive:0.36), (CANAGLIFLOZIN\METFORMIN HYDROCHLORIDE:Cardiac failure congestive:1.43), (CEFOTAXIME SODIUM:Cardiac failure congestive:0.49), (CEFPODOXIME PROXETIL:Cardiac failure congestive:0.07), (CHLORDIAZEPOXIDE HYDROCHLORIDE\CLIDINIUM BROMIDE:Cardiac failure congestive:0.38), (CIFENLINE:Cardiac failure congestive:0.15), (CILASTATIN SODIUM\IMIPENEM:Cardiac failure congestive:1.19), (CIMETIDINE:Cardiac failure congestive:1.4), (CLINDAMYCIN PHOSPHATE:Cardiac failure congestive:0.37), (CLOBAZAM:Cardiac failure congestive:0.09), (CLOMIPRAMINE HYDROCHLORIDE:Cardiac failure congestive:0.1), (COD LIVER OIL:Cardiac failure congestive:0.26), (CORTISONE\HYDROCORTISONE:Cardiac failure congestive:1.07), (CRIZOTINIB:Cardiac failure congestive:0.94), (CYPROHEPTADINE:Cardiac failure congestive:0.22), (DACLATASVIR:Cardiac failure congestive:0.37), (DACLIZUMAB:Cardiac failure congestive:0.84), (DANAZOL:Cardiac failure congestive:1.08), (DANTROLENE SODIUM:Cardiac failure congestive:0.06), (DASATINIB:Cardiac failure congestive:2.62), (DEFLAZACORT:Cardiac failure congestive:1.14), (DEGARELIX:Cardiac failure congestive:0.29), (DEXTROAMPHETAMINE SULFATE:Cardiac failure congestive:0.1), (DIMETHICONE\SILICON DIOXIDE\SIMETHICONE:Cardiac failure congestive:0.03), (DOLASETRON MESYLATE:Cardiac failure congestive:0.22), (ECONAZOLE:Cardiac failure congestive:0.67), (EDARAVONE:Cardiac failure congestive:0.31), (ELTROMBOPAG OLAMINE:Cardiac failure congestive:0.65), (EMPAGLIFLOZIN\METFORMIN HYDROCHLORIDE:Cardiac failure congestive:0.04), (ENALAPRIL MALEATE\HYDROCHLOROTHIAZIDE:Cardiac failure congestive:0.21), (ESTROGENS, CONJUGATED\MEDROXYPROGESTERONE ACETATE:Cardiac failure congestive:0.39), (ESTRONE\SUS SCROFA OVARY:Cardiac failure congestive:0.24), (ETORICOXIB:Cardiac failure congestive:0.18), (FENOTEROL HYDROBROMIDE:Cardiac failure congestive:0.38), (FENOTEROL HYDROBROMIDE\IPRATROPIUM BROMIDE:Cardiac failure congestive:0.72), (FERRIC HYDROXIDE:Cardiac failure congestive:0.76), (FINGOLIMOD HYDROCHLORIDE:Cardiac failure congestive:0.48), (FLUINDIONE:Cardiac failure congestive:0.79), (FLUVOXAMINE MALEATE:Cardiac failure congestive:0.39), (FORMOTEROL FUMARATE\MOMETASONE FUROATE:Cardiac failure congestive:0.77), (FUSIDIC ACID:Cardiac failure congestive:0.94), (GIMERACIL\OTERACIL\TEGAFUR:Cardiac failure congestive:0.24), (GLYCOPYRROLATE\INDACATEROL MALEATE:Cardiac failure congestive:0.36), (HUMAN PLATELET, ALLOGENIC:Cardiac failure congestive:0.05), (HYDROXYCHLOROQUINE DIPHOSPHATE:Cardiac failure congestive:0.1), (HYOSCYAMINE:Cardiac failure congestive:1.05), (IBRITUMOMAB TIUXETAN:Cardiac failure congestive:0.42), (IMATINIB MESYLATE:Cardiac failure congestive:1.6), (IMIPENEM:Cardiac failure congestive:0.2), (IMIPRAMINE HYDROCHLORIDE:Cardiac failure congestive:1.02), (IMMUNE GLOBULIN NOS:Cardiac failure congestive:0.3), (INDAPAMIDE\PERINDOPRIL:Cardiac failure congestive:1.47), (INTERFERON:Cardiac failure congestive:0.55), (INTERFERON ALFA-2A:Cardiac failure congestive:0.14), (IODINE:Cardiac failure congestive:0.22), (LERCANIDIPINE:Cardiac failure congestive:1.82), (LINSIDOMINE:Cardiac failure congestive:0.05), (LIOTHYRONINE:Cardiac failure congestive:0.32), (LIOTHYRONINE SODIUM:Cardiac failure congestive:0.62), (LISDEXAMFETAMINE DIMESYLATE:Cardiac failure congestive:0.56), (LITHIUM:Cardiac failure congestive:0.11), (LITHIUM CARBONATE:Cardiac failure congestive:1.38), (MEFENAMIC ACID:Cardiac failure congestive:0.11), (METHENAMINE HIPPURATE:Cardiac failure congestive:1.41), (METHYLPREDNISOLONE ACETATE:Cardiac failure congestive:0.19), (MICAFUNGIN:Cardiac failure congestive:0.28), (MOSAPRIDE CITRATE:Cardiac failure congestive:0.66), (NADROPARIN CALCIUM:Cardiac failure congestive:0.65), (NALOXONE HYDROCHLORIDE\TILIDINE HYDROCHLORIDE:Cardiac failure congestive:0.22), (NICARDIPINE:Cardiac failure congestive:1.19), (NIRAPARIB:Cardiac failure congestive:0.32), (NITROFURANTOIN\NITROFURANTOIN MONOHYDRATE:Cardiac failure congestive:0.56), (OLAPARIB:Cardiac failure congestive:0.04), (OLOPATADINE:Cardiac failure congestive:0.06), (OMEPRAZOLE SODIUM:Cardiac failure congestive:1), (ORPHENADRINE:Cardiac failure congestive:0.03), (OSELTAMIVIR:Cardiac failure congestive:0.21), (OXYMETAZOLINE:Cardiac failure congestive:0.37), (PANTOPRAZOLE MAGNESIUM:Cardiac failure congestive:1.76), (PARATHYROID HORMONE:Cardiac failure congestive:0.26), (PEGINTERFERON BETA-1A:Cardiac failure congestive:1.17), (PEGZEREPOETIN ALFA:Cardiac failure congestive:0.07), (PEMETREXED:Cardiac failure congestive:0.33), (PENICILLIN V:Cardiac failure congestive:0.39), (PENTOSAN POLYSULFATE SODIUM:Cardiac failure congestive:0.47), (PERTUZUMAB:Cardiac failure congestive:0.39), (PHENAZOPYRIDINE HYDROCHLORIDE:Cardiac failure congestive:0.17), (PIMECROLIMUS:Cardiac failure congestive:0.12), (PIROXICAM:Cardiac failure congestive:0.71), (POLYETHYLENE GLYCOL 3350\POTASSIUM CHLORIDE\SODIUM BICARBONATE\SODIUM CHLORIDE\SODIUM SULFATE ANHYDROUS:Cardiac failure congestive:0.14), (POLYETHYLENE GLYCOL 4000:Cardiac failure congestive:0.36), (PRASUGREL:Cardiac failure congestive:0.46), (PREDNISOLONE SODIUM PHOSPHATE:Cardiac failure congestive:0.17), (PROPAFENONE HYDROCHLORIDE:Cardiac failure congestive:1.38), (PSEUDOEPHEDRINE HYDROCHLORIDE:Cardiac failure congestive:0.19), (RADIATION THERAPY:Cardiac failure congestive:0.1), (RETINOL:Cardiac failure congestive:0.14), (RIZATRIPTAN:Cardiac failure congestive:0.32), (RIZATRIPTAN BENZOATE:Cardiac failure congestive:1.37), (ROSIGLITAZONE:Cardiac failure congestive:0.17), (SALMETEROL:Cardiac failure congestive:0.51), (SARPOGRELATE:Cardiac failure congestive:0.67), (SODIUM CITRATE:Cardiac failure congestive:0.13), (SODIUM FERROUS CITRATE:Cardiac failure congestive:0.04), (SORBITOL:Cardiac failure congestive:0.17), (SUMATRIPTAN\SUMATRIPTAN SUCCINATE:Cardiac failure congestive:1.56), (TENELIGLIPTIN:Cardiac failure congestive:0.04), (TERBINAFINE HYDROCHLORIDE:Cardiac failure congestive:1), (TESTOSTERONE UNDECANOATE:Cardiac failure congestive:0.23), (TETRACYCLINE:Cardiac failure congestive:1.32), (TINZAPARIN:Cardiac failure congestive:0.12), (TOBRAMYCIN:Cardiac failure congestive:0.55), (TOLTERODINE:Cardiac failure congestive:0.69), (TRIAMTERENE:Cardiac failure congestive:1.2), (UROKINASE:Cardiac failure congestive:0.24), (VARDENAFIL HYDROCHLORIDE TRIHYDRATE:Cardiac failure congestive:0.16), (VINORELBINE TARTRATE:Cardiac failure congestive:0.02), (YEAST:Cardiac failure congestive:0.12), (ZILEUTON:Cardiac failure congestive:0.6), (ZINC:Cardiac failure congestive:0.74), (DEVICE:Cardiac murmur:0.4), (DEXLANSOPRAZOLE:Cardiac murmur:0.13), (EVOLOCUMAB:Cardiac murmur:0.19), (ACETAMINOPHEN\CODEINE PHOSPHATE:Cardiac operation:0.02), (DICLOFENAC:Cardiac operation:0), (DICLOFENAC SODIUM:Cardiac operation:0.18), (ENOXAPARIN:Cardiac operation:0.06), (FLECAINIDE:Cardiac operation:0.06), (IBRUTINIB:Cardiac operation:0.64), (IRBESARTAN:Cardiac operation:0.19), (LERCANIDIPINE:Cardiac operation:0.21), (LINAGLIPTIN:Cardiac operation:0.14), (NAPROXEN:Cardiac operation:0.41), (OXYGEN:Cardiac operation:0.49), (SACUBITRIL\VALSARTAN:Cardiac operation:0.22), (CANDESARTAN CILEXETIL:Cardiac pacemaker insertion:0.15), (FLUTICASONE PROPIONATE:Cardiac pacemaker insertion:0.25), (LANSOPRAZOLE:Cardiac pacemaker insertion:0.21), (MELOXICAM:Cardiac pacemaker insertion:0.22), (NEBIVOLOL:Cardiac pacemaker insertion:0.35), (PANTOPRAZOLE:Cardiac pacemaker insertion:0.35), (PRAVASTATIN SODIUM:Cardiac pacemaker insertion:0.06), (RANOLAZINE:Cardiac pacemaker insertion:0.39), (SACUBITRIL\VALSARTAN:Cardiac pacemaker insertion:0.23), (DIGOXIN:Cardiac stress test:0.27), (ENALAPRIL:Cardiac stress test:0.17), (EVOLOCUMAB:Cardiac stress test:0.06), (PRAVASTATIN SODIUM:Cardiac stress test:0.06), (AMITRIPTYLINE:Cardiac valve disease:0.18), (ANASTROZOLE:Cardiac valve disease:0.26), (BENAZEPRIL HYDROCHLORIDE:Cardiac valve disease:0.16), (BUMETANIDE:Cardiac valve disease:0.13), (BUSPIRONE HYDROCHLORIDE:Cardiac valve disease:0.04), (CALCIUM CARBONATE\CHOLECALCIFEROL:Cardiac valve disease:0.35), (CANDESARTAN:Cardiac valve disease:0.3), (DASATINIB:Cardiac valve disease:0.09), (DEXLANSOPRAZOLE:Cardiac valve disease:0.34), (DILTIAZEM HYDROCHLORIDE:Cardiac valve disease:1.44), (DIPHENHYDRAMINE:Cardiac valve disease:0.04), (DOCUSATE SODIUM:Cardiac valve disease:0.47), (DOXAZOSIN\DOXAZOSIN MESYLATE:Cardiac valve disease:0.05), (FERROUS SULFATE:Cardiac valve disease:0.58), (FLECAINIDE:Cardiac valve disease:0.14), (FULVESTRANT:Cardiac valve disease:0.04), (ILOPROST:Cardiac valve disease:0.61), (INSULIN HUMAN:Cardiac valve disease:0.98), (LERCANIDIPINE:Cardiac valve disease:0.55), (LOVASTATIN:Cardiac valve disease:0.89), (METHYLPREDNISOLONE SODIUM SUCCINATE:Cardiac valve disease:0.04), (NEBIVOLOL:Cardiac valve disease:0.5), (PANTOPRAZOLE:Cardiac valve disease:0.79), (POLYETHYLENE GLYCOLS:Cardiac valve disease:0.05), (RABEPRAZOLE SODIUM:Cardiac valve disease:0.15), (RALOXIFENE HYDROCHLORIDE:Cardiac valve disease:0.01), (RANOLAZINE:Cardiac valve disease:0.75), (SECUKINUMAB:Cardiac valve disease:0.37), (SELEXIPAG:Cardiac valve disease:0.3), (TOCILIZUMAB:Cardiac valve disease:0.34), (PYRAZINAMIDE:Cardio-respiratory arrest:0.56), (ACARBOSE:Cardiomyopathy:0.26), (ANASTROZOLE:Cardiomyopathy:1.18), (BE NAZEPRIL\BENAZEPRIL HYDROCHLORIDE:Cardiomyopathy:0.22), (CALCIUM ACETATE:Cardiomyopathy:0.94), (CALCIUM CITRATE:Cardiomyopathy:0.18), (CERTOLIZUMAB PEGOL:Cardiomyopathy:0.56), (CILOSTAZOL:Cardiomyopathy:0.23), (CLINDAMYCIN\CLI NDAMYCIN PHOSPHATE:Cardiomyopathy:1.29), (CLOTRIMAZOLE:Cardiomyopathy:0.57), (CYCLOBEN ZAPRINE:Cardiomyopathy:0.3), (DALFAMPRIDINE:Cardiomyopathy:0.21), (DASATINIB:Car diomyopathy:1.08), (DRONABINOL:Cardiomyopathy:0.24), (EDOXABAN TOSYLATE:Cardiomyopathy:0.71), (FLUTICASONE:Cardiomyopathy:0.57), (FORMOTEROL FUMARATE:Cardiomyopathy:0.24), (FORMOTEROL FUMARATE DIHYDRATE\MOMETASONE FUROATE:Cardiomyopathy:0.04), (HYDROMORPHONE HYDROCHLORIDE:Cardiomyopathy:0.29), (HYDROXYZINE PAMOATE:Cardiomyopathy:0.69), (ILOPROST:Cardiomyopathy:0.82), (IMATINIB MESYLATE:Cardiomyopathy:0.77), (INDAPAMIDE\PERINDOPRIL:Cardiomyopathy:0.67), (I NSULIN DEGLUDEC:Cardiomyopathy:0.08), (LATANOPROST:Cardiomyopathy:0.45), (LEDIPASVIR\SOFOSBUVIR:Cardiomyopathy:0.88), (LERCANIDIPINE:Cardiomyopathy:1.24), (LISDEXAM FETAMINE DIMESYLATE:Cardiomyopathy:0.46), (LITHIUM CARBONATE:Cardiomyopathy:0.77), (METRONIDAZOLE\METRONIDAZOLE HYDROCHLORIDE:Cardiomyopathy:0.03), (NADOLOL:Cardiomyopathy:0.17), (NALOXONE HYDROCHLORIDE\OXYCODONE HYDROCHLORIDE:Cardiomyopathy:0.27), (NAPROXEN:Cardiomyopathy:1.4), (NORTRIPT YLINE:Cardiomyopathy:0.11), (OXCARBAZEPINE:Cardiomyopathy:0.58), (OXYBUTYNIN CHLORIDE:Cardiomyopathy:0.09), (PAZOPANIB HYDROCHLORIDE:Cardiomyopathy:0.16), (PENICILLIN:Cardiomyopathy:0.26), (PIOGLITA ZONE HYDROCHLORIDE:Cardiomyopathy:0.28), (PRAMIPEXOLE DIHYDROCHLORIDE:Cardiomyopathy:0.56), (PROBIOTICS NOS:Cardiomyopathy:0.09), (PROPAFENONE HYDROCHLORIDE:Cardiomyopathy:0.33), (QUETIAPINE FUMARATE:Cardiomyopathy:0.4), (RABEPRAZOLE:Cardiomyopathy:0.19), (ROFLUMILAS T:Cardiomyopathy:0.31), (ROSIGLITAZONE MALEATE:Cardiomyopathy:0.18), (SUMATRIPTAN\SUMATRIPTAN SUCCINATE:Cardiomyopathy:0), (TERBUTALINE SULFATE:Cardiomyopathy:0.19), (TETRACYCLINE:Cardiomyopathy:0.2), (THALIDOMIDE: Cardiomyopathy:0.36), (TIMOLOL:Cardiomyopathy:0.2), (TIOTROPIUM:Cardiomyopathy:0.44), (TIZANIDINE:Cardiomyopathy:0.26), (TRIAMCINOLONE:Cardiomyopathy:0), (TRIAMTER ENE:Cardiomyopathy:0.17), (VITAMIN B:Cardiomyopathy:0.55), (.ALPHA.1-PROTEINASE INHIBITOR HUMAN:Cardiovascular disorder:0.36), (ACEBUTOLOL HYDROCHLORIDE:Cardiovascular disorder:0.12), (ACETAMINOPHEN\CAFFEINE\OPIUM:Cardiovascular disorder:0.26), (ACETAMINOPHEN\PROPOXYPHENE HYDROCHLORIDE:Cardiovascular disorder:0.17), (ACTIVATED CHARCOAL:Cardiovascular disorder:0.05), (AGALSIDASE BETA:Cardiovascular disorder:0.72), (AMINO ACIDS\DEXTROSE\ELECTROLYTES NOS\SOYBEAN OIL\VITAMINS:Cardiovascular disorder:0.03), (AMLODIPINE BESYLATE\HYDROCHLOROTHIAZIDE\OLMESARTAN MEDOXOMIL:Cardiovascular disorder:0.34), (AMLODIPINE BESYLATE\OLMESARTAN MEDOXOMIL:Cardiovascular disorder:0.63), (AMLODIPINE BESYLATE\TELMISARTAN:Cardiovascular disorder:0.02), (AMLODIPINE\PERINDOPRIL:Cardiovascular disorder:0.29), (AMMONIUM LACTATE:Cardiovascular disorder:0.15), (ANTIHISTAMINES NOS:Cardiovascular disorder:0.02), (APREPITANT:Cardiovascular disorder:0.29), (ATOVAQUONE:Cardiovascular disorder:0.14), (AXITINIB:Cardiovascular disorder:0.48), (AZELASTINE:Cardiovascular disorder:0.25), (BICALUTAMIDE:Cardiovascular disorder:0.29), (BIFIDOBACTERIUM LONGUM SUBSP. INFANTIS:Cardiovascular disorder:0.3), (BROTIZOLAM:Cardiovascular disorder:0.22), (BUPRENORPHINE HYDROCHLORIDE:Cardiovascular disorder:0.6), (CALCITONIN:Cardiovascular disorder:0.23), (CALCITONIN SALMON:Cardiovascular disorder:0.29), (CALCIUM\MAGNESIUM:Cardiovascular disorder:0.14), (CALCIUM\MAGNESIUM\ZINC:Cardiovascular disorder:0.77), (CANAGLIFLOZIN\METFORMIN HYDROCHLORIDE:Cardiovascular disorder:1.3), (CAPSAICIN:Cardiovascular disorder:0.17), (CARBIMAZOLE:Cardiovascular disorder:0.24), (CEFAZOLIN:Cardiovascular disorder:0.16), (CEFTAZIDIME:Cardiovascular disorder:0.12), (CHLORDIAZEPOXIDE HYDROCHLORIDE\CLIDINIUM BROMIDE:Cardiovascular disorder:0.18), (CHLORPROMAZINE:Cardiovascular disorder:0.16), (CORTICOTROPIN:Cardiovascular disorder:0.58), (DACLIZUMAB:Cardiovascular disorder:0.93), (DANAZOL:Cardiovascular disorder:0.88), (DAPAGLIFLOZIN PROPANEDIOL\METFORMIN HYDROCHLORIDE:Cardiovascular disorder:0.22), (DEXAMETHASONE ACETATE:Cardiovascular disorder:0.44), (DEXTROAMPHETAMINE SULFATE:Cardiovascular disorder:0.16), (DICLOFENAC SODIUM\MISOPROSTOL:Cardiovascular disorder:0.19), (DIGITOXIN:Cardiovascular disorder:1.01), (DOXEPIN:Cardiovascular disorder:0.6), (DUTASTERIDE\TAMSULOSIN HYDROCHLORIDE:Cardiovascular disorder:0.61), (ECONAZOLE:Cardiovascular disorder:0.68), (ELOTUZUMAB:Cardiovascular disorder:0.24), (EMPAGLIFLOZIN\METFORMIN HYDROCHLORIDE:Cardiovascular disorder:0.17), (EPIRUBICIN:Cardiovascular disorder:0.17), (ESTROGENS, CONJUGATED\MEDROXYPROGESTERONE ACETATE:Cardiovascular disorder:0.85), (FENOFIBRIC ACID:Cardiovascular disorder:0.77), (FLUDROCORTISONE:Cardiovascular disorder:0.12), (FLUVOXAMINE MALEATE:Cardiovascular disorder:0.15), (FONDAPARINUX:Cardiovascular disorder:0.01), (FORMOTEROL FUMARATE\MOMETASONE FUROATE:Cardiovascular disorder:0.67), (FOSINOPRIL:Cardiovascular disorder:0.14), (FUSIDIC ACID:Cardiovascular disorder:0.72), (GLIMEPIRIDE\ROSIGLITAZONE MALEATE:Cardiovascular disorder:0.5), (GLYCOPYRROLATE\INDACATEROL MALEATE:Cardiovascular disorder:0.36), (HYDROXYCHLOROQUINE DIPHOSPHATE:Cardiovascular disorder:0.44), (HYDROXYZINE PAMOATE:Cardiovascular disorder:1.61), (HYOSCYAMINE:Cardiovascular disorder:0.35), (IDARUCIZUMAB:Cardiovascular disorder:0), (ILOPROST:Cardiovascular disorder:1.23), (IMIPRAMINE HYDROCHLORIDE:Cardiovascular disorder:0.47), (INSULIN DEGLUDEC:Cardiovascular disorder:0.81), (INSULIN PORK\INSULIN PURIFIED PORK:Cardiovascular disorder:0.24), (INTERFERON:Cardiovascular disorder:0.19), (INTERFERON ALFA-2A:Cardiovascular disorder:0.41), (ITRACONAZOLE:Cardiovascular disorder:0.37), (KETOROLAC\KETOROLAC TROMETHAMINE:Cardiovascular disorder:0.37), (LISDEXAMFETAMINE DIMESYLATE:Cardiovascular disorder:0.36), (MEPERIDINE HYDROCHLORIDE:Cardiovascular disorder:0.43), (MEPOLIZUMAB:Cardiovascular disorder:0.41), (METFORMIN HYDROCHLORIDE\ROSIGLITAZONE MALEATE:Cardiovascular disorder:0.82), (METHYLPHENIDATE HYDROCHLORIDE:Cardiovascular disorder:0.61), (METRONIDAZOLE\METRONIDAZOLE HYDROCHLORIDE:Cardiovascular disorder:0.77), (MICAFUNGIN:Cardiovascular disorder:0.4), (MINOCYCLINE HYDROCHLORIDE:Cardiovascular disorder:0.08), (MORPHINE SULFATE\NALTREXONE HYDROCHLORIDE:Cardiovascular disorder:0.11), (NALTREXONE:Cardiovascular disorder:0.18), (NITROFURANTOIN\NITROFURANTOIN MONOHYDRATE:Cardiovascular disorder:0.01), (ONABOTULINUMTOXINA:Cardiovascular disorder:0.29), (OSELTAMIVIR:Cardiovascular disorder:0.49), (OSELTAMIVIR PHOSPHATE:Cardiovascular disorder:0.01), (OXYBUTYNIN\OXYBUTYNIN CHLORIDE:Cardiovascular disorder:0.26), (PEGINTERFERON ALFA-2A:Cardiovascular disorder:0.11), (PEGINTERFERON BETA-1A:Cardiovascular disorder:1.01), (PEMETREXED:Cardiovascular disorder:0.04), (PEMETREXED DISODIUM:Cardiovascular disorder:0.96), (PILOCARPINE:Cardiovascular disorder:0.16), (PREDNISOLONE\PREDNISOLONE ACETATE\PREDNISOLONE SODIUM SUCCINATE:Cardiovascular disorder:0.82), (PYRIDOSTIGMINE BROMIDE:Cardiovascular disorder:0.5), (RETINOL:Cardiovascular disorder:0.17), (RIZATRIPTAN:Cardiovascular disorder:0.08), (ROPINIROLE:Cardiovascular disorder:0.65), (SACCHAROMYCES CEREVISIAE:Cardiovascular disorder:0.48), (SALMETEROL:Cardiovascular disorder:0.2), (SELEXIPAG:Cardiovascular disorder:0.47), (SULBACTAM SODIUM:Cardiovascular disorder:0.21), (TAFAMIDIS MEGLUMINE:Cardiovascular disorder:0.19), (TERBUTALINE:Cardiovascular disorder:0.23), (TESTOSTERONE UNDECANOATE:Cardiovascular disorder:0.7), (TETRACYCLINE:Cardiovascular disorder:1.06), (TICLOPIDINE:Cardiovascular disorder:0.2), (TINZAPARIN:Cardiovascular disorder:0.12), (TRIPTORELIN PAMOATE:Cardiovascular disorder:0.06), (VALGANCICLOVIR HYDROCHLORIDE:Cardiovascular disorder:1.55), (VILAZODONE HYDROCHLORIDE:Cardiovascular disorder:0.37), (VINCRISTINE SULFATE:Cardiovascular disorder:1), (ZAFIRLUKAST:Cardiovascular disorder:0.35), (ZILEUTON:Cardiovascular disorder:0.11), (ZONISAMIDE:Cardiovascular disorder:0.27), (.ALPHA.1-PROTEINASE INHIBITOR HUMAN:Cardiovascular event prophylaxis:0.67), (ABACAVIR SULFATE\DOLUTEGRAVIR SODIUM\LAMIVUDINE:Cardiovascular event prophylaxis:0.51), (ABOBOTULINUMTOXINA:Cardiovascular event prophylaxis:0.35), (ACETAMINOPHEN\DIPHENHYDRAMINE HYDROCHLORIDE:Cardiovascular event prophylaxis:0.04), (ACETAMINOPHEN\OXYCODONE HYDROCHLORIDE\OXYCODONE TEREPHTHALATE:Cardiovascular event prophylaxis:1.2), (ACITRETIN:Cardiovascular event prophylaxis:0.37), (ACTIVATED CHARCOAL:Cardiovascular event prophylaxis:0.61), (ADO-TRASTUZUMAB EMTANSINE:Cardiovascular event prophylaxis:1.52), (ALDESLEUKIN:Cardiovascular event prophylaxis:0.24), (ALEMTUZUMAB:Cardiovascular event prophylaxis:0.2), (ALUMINUM HYDROXIDE\MAGNESIUM HYDROXIDE\SIMETHICONE:Cardiovascular event prophylaxis:0.01), (AMBROXOL:Cardiovascular event prophylaxis:0.11), (AMINO ACIDS\DEXTROSE\ELECTROLYTES NOS:Cardiovascular event prophylaxis:0.69), (AMISULPRIDE:Cardiovascular event prophylaxis:1.21), (AMLODIPINE BESYLATE\IRBESARTAN:Cardiovascular event prophylaxis:0.05), (ANTIHISTAMINES NOS:Cardiovascular event prophylaxis:0.67), (ARIPIPRAZOLE LAUROXIL:Cardiovascular event prophylaxis:0.02), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Cardiovascular event prophylaxis:0.09), (ASPIRIN\CLOPIDOGREL:Cardiovascular event prophylaxis:0.4), (ATOVAQUONE:Cardiovascular event prophylaxis:0.7), (ATROPINE\DIPHENOXYLATE:Cardiovascular event prophylaxis:0.54), (BELATACEPT:Cardiovascular event prophylaxis:0.05), (BEPRIDIL:Cardiovascular event prophylaxis:0.17), (BETAMETHASONE DIPROPIONATE\CALCIPOTRIENE:Cardiovascular event prophylaxis:0.04), (BETAMETHASONE DIPROPIONATE\CALCIPOTRIENE\CALCIPOTRIENE HYDRATE:Cardiovascular event prophylaxis:0.66), (BETAMETHASONE SODIUM PHOSPHATE:Cardiovascular event prophylaxis:0.23), (BETAXOLOL HYDROCHLORIDE:Cardiovascular event prophylaxis:0.24), (BETHANECHOL CHLORIDE:Cardiovascular event prophylaxis:0.6), (BLINATUMOMAB:Cardiovascular event prophylaxis:0.98), (BOTULINUM TOXIN TYPE A:Cardiovascular event prophylaxis:0.12), (CALCIUM CARBONATE\CHOLECALCIFEROL\MAGNESIUM CARBONATE:Cardiovascular event prophylaxis:0.14), (CALCIUM\MAGNESIUM\ZINC:Cardiovascular event prophylaxis:1.03), (CARBOMER:Cardiovascular event prophylaxis:0.2), (CASANTHRANOL\DOCUSATE SODIUM:Cardiovascular event prophylaxis:0.42), (CEFOTAXIME\CEFOTAXIME SODIUM:Cardiovascular event prophylaxis:0.01), (CEFPODOXIME PROXETIL:Cardiovascular event prophylaxis:0.4), (CETIRIZINE HYDROCHLORIDE\PSEUDOEPHEDRINE HYDROCHLORIDE:Cardiovascular event prophylaxis:0.68), (CETUXIMAB:Cardiovascular event prophylaxis:0.9), (CHLOROTHIAZIDE:Cardiovascular event prophylaxis:0.19), (CHOLESTEROL:Cardiovascular event prophylaxis:0.02), (CLEMASTINE FUMARATE:Cardiovascular event prophylaxis:0.11), (CLIKSTAR:Cardiovascular event prophylaxis:0.39), (CLOMIPRAMINE HYDROCHLORIDE:Cardiovascular event prophylaxis:0.43), (CORTICOTROPIN:Cardiovascular event prophylaxis:1.03), (CORTISONE\HYDROCORTISONE:Cardiovascular event prophylaxis:2.36), (DABRAFENIB MESYLATE:Cardiovascular event prophylaxis:0.34), (DACLIZUMAB:Cardiovascular event prophylaxis:1.38), (DANAZOL:Cardiovascular event prophylaxis:0.96), (DAPSONE:Cardiovascular event prophylaxis:0.6), (DARUNAVIR\RITONAVIR:Cardiovascular event prophylaxis:0.21), (DEFLAZACORT:Cardiovascular event prophylaxis:1.72), (DESMOPRESSIN:Cardiovascular event prophylaxis:0.1), (DEXTROAMPHETAMINE SULFATE:Cardiovascular event prophylaxis:0.89), (DIPYRONE:Cardiovascular event prophylaxis:0.77), (DOBUTAMINE HYDROCHLORIDE:Cardiovascular event prophylaxis:0.28), (DOLASETRON MESYLATE:Cardiovascular event prophylaxis:0.61), (DOXYLAMINE SUCCINATE:Cardiovascular event prophylaxis:0.29), (ECONAZOLE:Cardiovascular event prophylaxis:1.65), (EDOXABAN TOSILATE MONOHYDRATE:Cardiovascular event prophylaxis:0.19), (ELTROMBOPAG OLAMINE:Cardiovascular event prophylaxis:0.02), (EMPAGLIFLOZIN\LINAGLIPTIN:Cardiovascular event prophylaxis:1.33), (ENALAPRIL MALEATE\LERCANIDIPINE HYDROCHLORIDE:Cardiovascular event prophylaxis:0.21), (ERIBULIN:Cardiovascular event prophylaxis:0.22), (ERLOTINIB:Cardiovascular event prophylaxis:0.14), (ESMOLOL HYDROCHLORIDE:Cardiovascular event prophylaxis:0.05), (ESOMEPRAZOLE MAGNESIUM\NAPROXEN:Cardiovascular event prophylaxis:0.35), (ESTRONE\SUS SCROFA OVARY:Cardiovascular event prophylaxis:0.34), (ETOPOSIDE PHOSPHATE:Cardiovascular event prophylaxis:0.09), (ETRETINATE:Cardiovascular event prophylaxis:0.32), (FENOFIBRIC ACID:Cardiovascular event prophylaxis:1.19), (FERROUS SULFATE\GLYCINE:Cardiovascular event prophylaxis:0.39), (FEXOFENADINE\PSEUDOEPHEDRINE:Cardiovascular event prophylaxis:0.71), (FLURBIPROFEN:Cardiovascular event prophylaxis:0.12), (FLUTAMIDE:Cardiovascular event prophylaxis:0.01), (FONDAPARINUX SODIUM:Cardiovascular event prophylaxis:0.79), (FORMOTEROL FUMARATE\MOMETASONE FUROATE:Cardiovascular event prophylaxis:0.66), (FOSFOMYCIN:Cardiovascular event prophylaxis:0.15), (GEFITINIB:Cardiovascular event prophylaxis:0.43), (GIMERACIL\OTERACIL\TEGAFUR:Cardiovascular event prophylaxis:0.87), (GLIMEPIRIDE\ROSIGLITAZONE MALEATE:Cardiovascular event prophylaxis:0.38), (GLIPIZIDE\METFORMIN HYDROCHLORIDE:Cardiovascular event prophylaxis:0.17), (GLYCOPYRROLATE\INDACATEROL MALEATE:Cardiovascular event prophylaxis:0.59), (GUANFACINE HYDROCHLORIDE:Cardiovascular event prophylaxis:0.5), (HERBALS\TURMERIC:Cardiovascular event prophylaxis:1.38), (HYDROXYCHLOROQUINE DIPHOSPHATE:Cardiovascular event prophylaxis:1.06), (IBRITUMOMAB TIUXETAN:Cardiovascular event prophylaxis:0.46), (ILOPROST:Cardiovascular event prophylaxis:1.65), (INFLIXIMAB-DYYB:Cardiovascular event prophylaxis:0.2), (INTERFERON:Cardiovascular event prophylaxis:0.85), (INTERFERON ALFA-2A:Cardiovascular event prophylaxis:1.13), (INTERFERON ALFA-2B:Cardiovascular event prophylaxis:0.75), (IXAZOMIB:Cardiovascular event prophylaxis:0.83), (LACTASE:Cardiovascular event prophylaxis:0.83), (LACTOBACILLUS ACIDOPHILUS\LACTOBACILLUS DELBRUECKII SUBSP. BULGARICUS:Cardiovascular event prophylaxis:0.26), (LANREOTIDE:Cardiovascular event prophylaxis:0.12), (LAPATINIB DITOSYLATE:Cardiovascular event prophylaxis:0.72), (LENVATINIB:Cardiovascular event prophylaxis:0.44), (LEVOLEUCOVORIN CALCIUM:Cardiovascular event prophylaxis:0.31), (LOTEPREDNOL ETABONATE:Cardiovascular event prophylaxis:0.84), (LUTETIUM LU-177:Cardiovascular event prophylaxis:0.13), (MAGNESIUM ASPARTATE\POTASSIUM ASPARTATE:Cardiovascular event prophylaxis:0.23), (MEPOLIZUMAB:Cardiovascular event prophylaxis:0.49), (METFORMIN HYDROCHLORIDE\ROSIGLITAZONE MALEATE:Cardiovascular event prophylaxis:0.5), (METFORMIN HYDROCHLORIDE\SAXAGLIPTIN:Cardiovascular event prophylaxis:0.15), (METFORMIN HYDROCHLORIDE\SAXAGLIPTIN HYDROCHLORIDE:Cardiovascular event prophylaxis:0.51), (METFORMIN PAMOATE:Cardiovascular event prophylaxis:0.19), (METHENOLONE ENANTHATE:Cardiovascular event prophylaxis:0.22), (METRELEPTIN:Cardiovascular event prophylaxis:0.26), (MEXILETINE HYDROCHLORIDE:Cardiovascular event prophylaxis:0.29), (MIDODRINE HYDROCHLORIDE:Cardiovascular event prophylaxis:0.88), (MOEXIPRIL HYDROCHLORIDE:Cardiovascular event prophylaxis:0.55), (MORPHINE SULFATE\NALTREXONE HYDROCHLORIDE:Cardiovascular event prophylaxis:0.75), (NALOXONE:Cardiovascular event prophylaxis:0.4), (NARATRIPTAN HYDROCHLORIDE:Cardiovascular event prophylaxis:0.11), (NEOSTIGMINE:Cardiovascular event prophylaxis:0.08), (NIRAPARIB:Cardiovascular event prophylaxis:0.96), (NOREPINEPHRINE:Cardiovascular event prophylaxis:0.59), (NOREPINEPHRINE BITARTRATE:Cardiovascular event prophylaxis:0.82), (OCTREOTIDE:Cardiovascular event prophylaxis:0.01), (OSELTAMIVIR:Cardiovascular event prophylaxis:0.81), (OSIMERTINIB:Cardiovascular event prophylaxis:0.2), (OXYBUTYNIN\OXYBUTYNIN CHLORIDE:Cardiovascular event prophylaxis:0.47), (PALIPERIDONE PALMITATE:Cardiovascular event prophylaxis:0.42), (PANITUMUMAB:Cardiovascular event prophylaxis:0.35), (PARICALCITOL:Cardiovascular event prophylaxis:0.76), (PATIROMER:Cardiovascular event prophylaxis:0.97), (PEGASPARGASE:Cardiovascular event prophylaxis:0.12), (PENICILLIN G:Cardiovascular event prophylaxis:0.41), (PENTOSTATIN:Cardiovascular event prophylaxis:0.48), (PERPHENAZINE:Cardiovascular event prophylaxis:0.02), (PHENAZOPYRIDINE:Cardiovascular event prophylaxis:0.2), (PILSICAINIDE HYDROCHLORIDE:Cardiovascular event prophylaxis:0.61), (PIMECROLIMUS:Cardiovascular event prophylaxis:0.25), (POLYETHYLENE GLYCOL 3350\POTASSIUM CHLORIDE\SODIUM BICARBONATE\SODIUM CHLORIDE\SODIUM SULFATE ANHYDROUS:Cardiovascular event prophylaxis:0.56), (PORFIMER SODIUM:Cardiovascular event prophylaxis:0.09), (PRAMLINTIDE ACETATE:Cardiovascular event prophylaxis:0.13), (PREDNISOLONE SODIUM PHOSPHATE:Cardiovascular event prophylaxis:0.36), (PROTRIPTYLINE HYDROCHLORIDE:Cardiovascular event prophylaxis:0.09), (PYRAZINAMIDE:Cardiovascular event prophylaxis:0.76), (PYRIDOSTIGMINE BROMIDE:Cardiovascular event prophylaxis:0.97), (RADIATION THERAPY:Cardiovascular event prophylaxis:1.48), (RAMUCIRUMAB:Cardiovascular event prophylaxis:0.59), (RILPIVIRINE HYDROCHLORIDE:Cardiovascular event prophylaxis:0.19), (RIZATRIPTAN:Cardiovascular event prophylaxis:0.44), (RIZATRIPTAN BENZOATE:Cardiovascular event prophylaxis:1.23), (ROSIGLITAZONE MALEATE:Cardiovascular event prophylaxis:0.71), (SALMETEROL:Cardiovascular event prophylaxis:0.69), (SELEGILINE:Cardiovascular event prophylaxis:0.35), (SELEGILINE HYDROCHLORIDE:Cardiovascular event prophylaxis:0.21), (SELEXIPAG:Cardiovascular event prophylaxis:0.89), (SILVER SULFADIAZINE:Cardiovascular event prophylaxis:0.59), (SODIUM PHOSPHATE, DIBASIC\SODIUM PHOSPHATE, MONOBASIC:Cardiovascular event prophylaxis:0.84), (SOFOSBUVIR\VELPATASVIR:Cardiovascular event prophylaxis:0.29), (SUFENTANIL CITRATE:Cardiovascular event prophylaxis:0.26), (SUMATRIPTAN\SUMATRIPTAN SUCCINATE:Cardiovascular event prophylaxis:1.6), (TAFLUPROST:Cardiovascular event prophylaxis:0.2), (TAZOBACTAM:Cardiovascular event prophylaxis:0.49), (TENOFOVIR:Cardiovascular event prophylaxis:0.16), (THEOPHYLLINE:Cardiovascular event prophylaxis:0.14), (THIOPENTAL SODIUM:Cardiovascular event prophylaxis:0.04), (TICLOPIDINE:Cardiovascular event prophylaxis:0.53), (TILIDINE:Cardiovascular event prophylaxis:0.09), (TIMOLOL MALEATE:Cardiovascular event prophylaxis:1.23), (TRABECTEDIN:Cardiovascular event prophylaxis:0.18), (TRAMETINIB DIMETHYL SULFOXIDE:Cardiovascular event prophylaxis:0.25), (TRIHEXYPHENIDYL:Cardiovascular event prophylaxis:0.11), (TRIMIPRAMINE MALEATE:Cardiovascular event prophylaxis:0.6), (TROSPIUM CHLORIDE:Cardiovascular event prophylaxis:1.46), (UMECLIDINIUM BROMIDE:Cardiovascular event prophylaxis:0.93), (VASOPRESSIN:Cardiovascular event prophylaxis:0.18), (VECURONIUM BROMIDE:Cardiovascular event prophylaxis:0.01), (VINORELBINE\VINORELBINE TARTRATE:Cardiovascular event prophylaxis:0.46), (XIPAMIDE:Cardiovascular event prophylaxis:0.1), (YEAST:Cardiovascular event prophylaxis:0.26), (ZILEUTON:Cardiovascular event prophylaxis:0.35), (ZINC:Cardiovascular event prophylaxis:1.38), (ZONISAMIDE:Cardiovascular event prophylaxis:0.86), (AMIODARONE:Carotid artery stenosis:0.35), (AMIODARONE HYDROCHLORIDE:Carotid artery stenosis:0.09), (BUMETANIDE:Carotid artery stenosis:0.04), (CALCIUM CARBONATE\CHOLECALCIFEROL:Carotid artery stenosis:0.04), (RANOLAZINE:Carotid artery stenosis:0.13), (SACUBITRIL\VALSARTAN:Carotid artery stenosis:0.53), (UNSPECIFIED INGREDIENT:Carotid artery stenosis:0.39), (AMIODARONE:Carpal tunnel syndrome:0.01), (BISOPROLOL FUMARATE:Carpal tunnel syndrome:0.08), (CALCIUM CARBONATE\CHOLECALCIFEROL:Carpal tunnel syndrome:0.28), (CERTOLIZUMAB PEGOL:Carpal tunnel syndrome:0.67), (DALFAMPRIDINE:Carpal tunnel syndrome:0.45), (ENALAPRIL:Carpal tunnel syndrome:0.48), (EVOLOCUMAB:Carpal tunnel syndrome:0.83), (FERROUS SULFATE:Carpal tunnel syndrome:0.21), (ILOPROST:Carpal tunnel syndrome:0.12), (IRON:Carpal tunnel syndrome:0.52), (LISDEXAMFETAMINE DIMESYLATE:Carpal tunnel syndrome:0.12), (MAGNESIUM:Carpal tunnel syndrome:0.29), (METHYLPREDNISOLONE SODIUM SUCCINATE:Carpal tunnel syndrome:0.15), (OXCARBAZEPINE:Carpal tunnel syndrome:0.33), (PRAVASTATIN SODIUM:Carpal tunnel syndrome:0.42), (RAMIPRIL:Carpal tunnel syndrome:0.53), (SACUBITRIL\VALSARTAN:Carpal tunnel syndrome:0.23), (TAMSULOSIN:Carpal tunnel syndrome:0.29), (VERAPAMIL HYDROCHLORIDE:Carpal tunnel syndrome:0.53), (.ALPHA.-TOCOPHEROL ACETATE, D-\ASCORBIC ACID\CALCIUM PANTOTHENATE\FOLIC ACID\MANGANESE\NIACINAMIDE\PYRIDOXINE\RIBOFLAVIN\THIAMINE\VITAMIN A\ZINC OXIDE:Cataplexy:0.13), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Cataplexy:1.76), (.ALPHA.-TOCOPHEROL\VITAMINS:Cataplexy:0.33), (25-HYDROXYCHOLECALCIFEROL, 5-TRANS-:Cataplexy:0.65), (ABOBOTULINUMTOXINA:Cataplexy:0.24), (ACARBOSE:Cataplexy: 0.54), (ACETAMINOPHEN\ASPIRIN\CAFFEINE\CODEINE PHOSPHATE:Cataplexy:0.11), (ACETAMINOPHEN\CHLORPHENIRAMINE MALEATE\PSEUDOEPHEDRINE HYDROCHLORIDE:Cataplexy:0.39), (ACETAMINOPHEN\DIPHENHYDRAMINE\DIPHEN HYDRAMINE CITRATE:Cataplexy:0.16), (ACETAMINOPHEN\METHOCARBAMOL:Cataplexy:0.01), (ACI TRETIN:Cataplexy:0.32), (ACRIVASTINE\PSEUDOEPHEDRINE HYDROCHLORIDE:Cataplexy:0.37), (AJMALINE:Cataplexy:0.11), (ALEMTUZUMAB:Cataplexy: 0.31), (ALENDRONIC ACID:Cataplexy:0.72), (ALFACALCIDOL:Cataplexy:0.12), (ALLANTOIN\BENZOCAINE\CA MPHOR (NATURAL)\DIMETHICONE\MENTHOL:Cataplexy:0.65), (ALUMINUM HYDROXIDE\MAGNESIUM HYDROXIDE\SIMETHICONE:Cataplexy:0.36), (AMINO ACIDS\DEXTROSE\ELECTROLYTES NOS:Cataplexy:0.71), (AMISULPRIDE:Cataplexy:0.64), (AMITRIPTYLINE\PERPHENAZINE:Cataplexy:0.27), (AMPHETAMINE\DEXTROAMPHETAMINE:Cataplexy:0.23), (ARIPIPRAZ OLE LAUROXIL:Cataplexy:0.87), (ASCORBIC ACID/CHROMIUM/SELENIUM/VANADYL SULFATE:Cataplexy:1.01), (ASPIRIN\CAFFEINE\SALICYLAMIDE:Cataplexy:1.07), (ASPIRI N\DIHYDROXYALUMINUM AMINOACETATE ANHYDROUS\MAGNESIUM CARBONATE:Cataplexy:0.25), (ATENOLOL\HYDROCHLOROTHIAZIDE:Cataplexy:0.29), (AVANAFIL:Cataplexy:0.82), (AZITHROMYCIN MONOHYDRATE:Cataplexy:0.54), (BAMIFYLLINE HYDROCHLORIDE:Cataplexy:0.14), (BAZEDOXIFENE ACETATE\ESTROGENS, CONJUGATED:Cataplexy:0.47), (BECLOMETHASONE\FORMOTEROL:Cataplexy:0.2), (BE NAZEPRIL\BENAZEPRIL HYDROCHLORIDE:Cataplexy:1.6), (BETAMETHASONE\CALCIPOTRIENE:Cataplexy:0.87), (BIFIDOBACTERIUM LONGUM\LACTOBACILLUS ACIDOPHILUS\LACTOBACILLUS RHAMNOSUS:Cataplexy:00.01), (BORTEZOMIB:Cataplexy:0.16), (BRINZOLAMIDE:Cataplexy: 1.32), (BRIVARACETAM:Cataplexy:0.24), (BROMAZEPAM:Cataplexy:0.37), (BUDESONID E\FORMOTEROL:Cataplexy:0.08), (BUPRENORPHINE HYDROCHLORIDE\NALOXONE HYDROCHLORIDE DIHYDRATE:Cataplexy:0.12), (BUPRENORPHINE\NALOXONE:Cataplexy:1.48), (BUTABA RBITAL:Cataplexy:0.98), (BUTYLSCOPOLAMINE BROMIDE:Cataplexy:0.46), (CABOZANTINIB S-MALATE:Cataplexy:0.03), (CAFFEINE\DIPYRONE\ORPHENADRINE:Cataplexy:0.15), (CAL CITONIN:Cataplexy:0.7), (CALCIUM CARBONATE\ERGOCALCIFEROL:Cataplexy:0.1), (CALCIUM CARBONATE\ERGOCALCIFEROL\RETINOL:Cataplexy:0.32), (CALCIUM CARBONATE\ETIDRONATE DISODIUM:Cataplexy:0.18), (CAMPHOR\DIPHENHYDRAMINE\ZINC OXIDE:Cataplexy:0.14), (CANAKINUMAB:Cataplexy:0.98), (CANNABIDIOL:Cataplexy:0.06), (CANNABIS SATIVA SUBSP. SATIVA FLOWERING TOP:Cataplexy:0.03), (CAPSICUM:Cataplexy:0.37), (CARBOXYMETHYLCELLULOSE SODIUM\GLYCERIN\POLYSORBATE 80:Cataplexy:0.41), (CARFILZOMIB:Cataplexy:0.78), (CEFAZOLIN SODIUM:Cataplexy:0.2), (CERIVASTATIN SODIUM:Cataplexy:0.88), (CHLORMEZANONE:Cataplexy:0.14), (CHLOROTHIAZIDE:Cataplexy: 0.15), (CHLORPHENIRAMINE MALEATE\IBUPROFEN\PSEUDOEPHEDRINE HYDROCHLORIDE:Cataplexy:0.51), (CHLORPROTHIXENE:Cataplexy:0.14), (CHOLECALC IFEROL\GENISTEIN\ZINC GLYCINATE:Cataplexy:0.17), (CHOLINE BITARTRATE:Cataplexy:0.07), (CICLOPIROX OLAMINE:Cataplexy:0.45), (CISAPRIDE:Cataplexy:0.07), (CLIKSTAR:Cataplexy:0.35), (COD EINE PHOSPHATE\PROMETHAZINE:Cataplexy:0.19), (CRANBERRY JUICE:Cataplexy:0.57), (DACLIZUMAB:Cataplexy:1.62), (DALTEPARIN:Cataplexy:0.4), (DA NTROLENE SODIUM:Cataplexy:0.32), (DAPAGLIFLOZIN PROPANEDIOL\METFORMIN HYDROCHLORIDE:Cataplexy:0.14), (DASABUVIR\OMBITASVIR\PARITAPREVIR\RITO NAVIR:Cataplexy:0.61), (DEFLAZACORT:Cataplexy:0.91), (DEHYDROEPIANDROSTERON E-3-SULFATE SODIUM:Cataplexy:0.4), (DESOGESTREL:Cataplexy:0.34), (DEVICE:Cataplexy:0.89), (DICH LORPHENAMIDE:Cataplexy:0.26), (DIMETHICONE 410:Cataplexy:0.66), (DIMETHYL SULFOXIDE:Cataplexy:1.03), (DIPHENHYDRAMINE HYDROCHLORIDE\DOXYLAMINE SUCCINATE:Cataplexy:0.26), (DIPYRONE:Cataplexy:0.85), (DIPYRONE\PROMETHAZINE HYDROCHLORIDE:Cataplexy:0.93), (DOLASETRON MESYLATE:Cataplexy:0.31), (DOTHIEPIN\DOTHIEPIN HYDROCHLORIDE:Cataplexy:0.2), (DOXYLAMINE SUCCINATE\PYRIDOXINE HYDROCHLORIDE:Cataplexy:0.01), (ECALLANTIDE:Cataplexy:0.26), (EDOXABAN TOSYLATE:Cataplexy:0.86), (ELTROMBOPAG OLAMINE:Cataplexy:0.21), (EPHEDRINE HYDROCHLORIDE\GUAIFENESIN:Cataplexy:0.86), (ESSENTIAL OILS:Cataplexy:0.08), (ESTRADIOL ACETATE:Cataplexy:1.35), (ETHACRYNIC ACID:Cataplexy:0.46), (ETHINYL ESTRADIOL\GESTODENE:Cataplexy:0.36), (ETORICOXIB:Cataplexy:0.18), (EUCALYPTOL \MENTHOL\METHYL SALICYLATE\THYMOL:Cataplexy:0.16), (EVEROLIMUS:Cataplexy:0.27), (FELBAMATE:Cataplexy: 0.35), (FERROUS BISGLYCINATE:Cataplexy:0.87), (FLIBANSERIN:Cataplexy:0.31), (FLUNARIZINE HYDROCHLORIDE:Cataplexy:0.12), (FLUOXETINE HYDROCHLORIDE\OLANZAPINE:Cataplexy:1.45), (FLUTICASONE FUROATE\SALMETEROL XINAFOATE:Cataplexy:0.99), (FORMOTEROL:Cataplexy:0.24), (FORMOTEROL FUMARATE\MOMETASONE FUROATE:Cataplexy:0.79), (FUSIDIC ACID:Cataplexy:0.98), (GLYCOPYRROLATE\INDACATEROL MALEATE:Cataplexy:0.36), (GLYCOPYRRONIUM\INDACATEROL:Cataplexy:0.4), (GUAIF ENESIN\PHENYLEPHRINE HYDROCHLORIDE:Cataplexy:0.39), (HERBALS\HOMEOPATHICS\GOLDENSEAL:Cataplexy: 0.5), (HERBALS\VITIS VINIFERA SEED:Cataplexy:0.3), (HIGH DENSITY POLYETHYLENE:Cataplexy:0.46), (HORSE CHESTNUT:Cataplexy:0.97), (HUMAN C1-ESTERASE INHIBITOR:Cataplexy:0.06), (HYDROCHLORIC ACID:Cataplexy:0.18), (HYPERICUM PERFORATUM:Cataplexy:0.29), (IBRUTINIB:Cataplexy:0.56), (ILOPROST:Cataplexy:0.28), (I NFLIXIMAB-DYYB:Cataplexy:0.36), (INSULIN BEEF:Cataplexy:0.63), (INTERFERON:Cataplexy:0.35), (INTERFERON GAMMA-1B:Cataplexy:0.56), (IRON\POLYSACCHARIDES:Cataplexy:0.12), (ISOPROPYL ALCOHOL\LEUPROLIDE ACETATE\NORETHINDRONE ACETATE:Cataplexy:0.89), (ITRACONAZOLE\SODIUM CHLORIDE:Cataplexy:0.02), (IXAZOMIB:Cataplexy:0.18), (LACTOBACILLUS ACIDOPHILUS\LACTOBACILLUS DELBRUECKII SUBSP. BULGARICUS:Cataplexy:0.11), (LACTOPEROXIDASE BOVINE\LYSOZYME HYDROCHLORIDE:Cataplexy:0.07), (LEVOBUNOLOL:Cataplexy:0.06), (LEVOMEFOLIC ACID\METHYLCOBALAMIN\TURMERIC:Cataplexy:0.32), (LOFEPRAMINE:Cataplexy:0.01), (LOMITAPIDE MESYLATE:Cataplexy:1.61), (LOVASTATIN\NIACIN:Cataplexy:0.2), (MATRICARIA RECUTITA:Cataplexy:0.58), (MECLOZINE DIHYDROCHLORIDE:Cataplexy:0.63), (MENTHOL\ZINC OXIDE:Cataplexy:0.61), (MEPOLIZUMAB:Cataplexy:0.57), (METFORMIN HYDROCHLORIDE\SAXAGLIPTIN HYDROCHLORIDE:Cataplexy:0.24), (METHENAMINE MANDELATE:Cataplexy:1.08), (MOEXIPRIL HYDROCHLORIDE:Cataplexy:0.95), (NALOXONE HYDROCHLORIDE\OXYCODONE HYDROCHLORIDE:Cataplexy:0.89), (NAPHAZOLINE HYDROCHLORIDE:Cataplexy:0.49), (NEFAZODONE HYDROCHLORIDE:Cataplexy:1.02), (NILOTINIB:Cataplexy:1.22), (NINTEDANIB:Cataplexy: 0.4), (NIRAPARIB:Cataplexy:0.22), (OLODATEROL HYDROCHLORIDE\TIOTROPIUM BROMIDE MONOHYDRATE:Cataplexy:0.61), (OMEGA-6 FATTY ACIDS:Cataplexy:0.23), (OXYBUTYNIN\OXYBUTYNIN CHLORIDE:Cataplexy:1.34), (OXYQUINOLINE SULFATE\SODIUM LAURYL SULFATE:Cataplexy:0.39), (PAMIDRONATE DISODIUM:Cataplexy:0.69), (PAPAYA:Cataplexy:1.14), (PARATHYROID HORMONE:Cataplexy:0.54), (PARSLEY:Cataplexy:0.13), (PASSIFLORA INCARNATA FLOWER:Cataplexy:0.72), (PATIROMER:Cataplexy:0.76), (PAZOPANIB HYDROCHLORIDE:Cataplexy:0.44), (PEGINTERFERON BETA-1A:Cataplexy:1.1), (PENICILLIN V:Cataplexy:0.03), (PERAMPANEL:Cataplexy:0.41), (PINAVERIUM BROMIDE:Cataplexy:0.07), (PODOFILOX:Cataplexy:0.21), (PONATINIB:Cataplexy:1), (POTA SSIUM BITARTRATE:Cataplexy:0.46), (PREDNISOLONE ACETATE\SULFACETAMIDE SODIUM:Cataplexy:0.2), (PROPOXYPHENE:Cataplexy:0.07), (QUINAPRIL\QUINAPRIL HYDROCHLORIDE:Cataplexy:1.03), (QUINIDINE GLUCONATE:Cataplexy:0.8), (RABEPRAZOLE:Cataplexy:1.57), (RASAGILINE MESYLATE:Cataplexy:0.19), (RED CLOVER:Cataplexy:0.79), (RILPIVIRINE HYDROCHLORIDE:Cataplexy:0.04), (ROFECOXIB:Cataplexy:0.72), (RUFINAMIDE:Cataplexy: 0.03), (SALMETEROL:Cataplexy:0.26), (SAPROPTERIN DIHYDROCHLORIDE:Cataplexy:0.13), (SIBUTRAMINE HYDROCHLORIDE:Cataplexy:1.33), (SODIUM HYPOCHLORITE:Cataplexy:0.2), (SODIUM PHOSPHATE, DIBASIC\SODIUM PHOSPHATE, MONOBASIC:Cataplexy:0.38), (SOFOSBUVIR\VELPATASVIR:Cataplexy:0.59), (STRONTIU M:Cataplexy:0.03), (SUNITINIB MALATE:Cataplexy:0.44), (TEDUGLUTIDE\WATER:Cataplexy:0.82), (TERBUTALINE SULFATE:Cataplexy:1.73), (TESAMORELIN:Cataplexy:0.28), (THALIDOMIDE:Cataplexy:0.89), (THEOPHYLLINE:Cataplexy:0.77), (THIOTHIXENE:Cataplexy:0.43), (TIBOLONE:Cataplexy: 0.95), (TIOCONAZOLE:Cataplexy:0.03), (TIOPRONIN:Cataplexy:0.25), (TIOTROPIUM:Cataplexy: 0.65), (TOLMETIN:Cataplexy:0.13), (TOLTERODINE:Cataplexy:0.86), (TRIENTINE HYDROCHLORIDE:Cataplexy:0.22), (TRIFLUOPERAZINE HYDROCHLORIDE:Cataplexy:0.62), (TRIMETHADIONE:Cataplexy:0.42), (TRIPTORELIN PAMOATE:Cataplexy:0.01), (TROSPIUM:Cataplexy:0.05), (VALDECOXIB:Cataplexy:0.52), (V ALGANCICLOVIR HYDROCHLORIDE:Cataplexy:0.83), (VISMODEGIB:Cataplexy:0.06), (VITAMIN A PALMITATE:Cataplexy:0.39), (ZEAXANTHIN:Cataplexy:0.08), (ZICONOTIDE ACETATE:Cataplexy:0.89), (ACENOCOUMAROL:Cataract:0.07), (ACETAMINOPHEN\OXY CODONE\OXYCODONE HYDROCHLORIDE:Cataract:0.01), (ACETAMINOPHEN\TRAMADOL:Cataract:0.22), (BICA LUTAMIDE:Cataract:0.41), (BUDESONIDE\FORMOTEROL FUMARATE:Cataract:0.22), (BUPROPION HYDROCHLORIDE:Cataract:0.8), (BUSPIRONE HYDROCHLORIDE:Cataract:0.08), (CALCITRIOL:Cataract:0.78), (CALCIUM CARBONATE\CHOLECALCIFEROL\MAGNESIUM CARBONATE:Cataract:0.26), (CANAKINUMAB:Cataract:0.14), (CARVEDILOL PHOSPHATE:Cataract:0.25), (CHOLESTYRAMINE:Cataract:0.63), (CILASTATIN SODIUM\IMIPENEM:Cataract:0.24), (CYCLOPENTOLATE:Cataract:0.49), (DAPAGLIFLOZI N PROPANEDIOL:Cataract:0.25), (DASATINIB:Cataract:1.09), (DESLORATADINE:Cataract:0.36), (DEVICE:Cataract:0.8), (DRONABINOL:Cataract:0.07), (ELETRIPTAN HYDROBROMIDE:Cataract:0.02), (ETORICOXIB:Cataract:0.21), (EVEROLIMUS:Cataract:0.95), (EXEMESTANE:Cataract:0.05), (EZETIMIBE\SIMVASTATIN:Cataract:0.06), (FESOTERO DINE FUMARATE:Cataract:0.48), (FLECAINIDE:Cataract:0.48), (FLUTICASONE FUROATE\SALMETEROL XINAFOATE:Cataract:0.17), (FLUTICASONE FUROATE\VILANTEROL TRIFENATATE:Cataract:0.34), (FLUTICASONE\FLUTICASONE PROPIONATE:Cataract:0.29), (FORMOTEROL FUMARATE DIHYDRATE\MOMETASONE FUROATE:Cataract:0.09), (FULVESTRANT:Cataract:0.46), (GLYCOPYRRONIUM\ANDACA TEROL:Cataract:0.22), (HYDROCHLOROTHIAZIDE\IRBESARTAN:Cataract:0.35), (HYDRO CHLOROTHIAZIDE\LISINOPRIL:Cataract:0.77), (HYDROXYCHLOROQUINE SULFATE:Cataract:0.66), (HYDROXYZINE PAMOATE:Cataract:0.1), (INDAPAMIDE\PERINDOPRIL:Cataract:0.44), (INSULIN DEGLUDEC:Cataract:0.06), (INSULIN GLULISINE:Cataract:0.12), (IPRATROPIUM:Cataract:0.56), (ISOSORBIDE:Cataract:0.7), (LE DIPASVIR\SOFOSBUVIR:Cataract:0.26), (LERCANIDIPINE:Cataract:0.88), (LEVALBUTER OL HYDROCHLORIDE:Cataract:0.44), (LIDOCAINE\PRILOCAINE:Cataract:0.22), (LITHIUM CARBONATE:Cataract:0.43), (METAXALONE:Cataract:0.07), (METHADONE HYDROCHLORIDE:Cataract:0.1), (METHENAMINE HIPPURATE:Cataract:0.1), (METHENOLONE ENANTHATE:Cataract:0), (METHIMAZOLE:Cataract:0.22), (METHYLPREDNISOLONE ACETATE:Cataract:0.14), (METHYLPREDNISOLONE SODIUM SUCCINATE:Cataract:1.19), (MIDODRINE:Cataract:0.04), (MINOCYCLINE HYDROCHLORIDE:Cataract:0.02), (MINOCYCLINE\MINOCYCLINE HYDROCHLORIDE:Cataract:0.38), (NEBIVOLOL:Cataract:0.68), (NEBIVOLOL HYDROCHLORIDE:Cataract:0.39), (NORTRIPTYLINE:Cataract:0.05), (OLANZAPINE:Cataract: 0.59), (OLODATEROL HYDROCHLORIDE\TIOTROPIUM BROMIDE MONOHYDRATE:Cataract:0.27), (OMEGA-3-ACID ETHYL ESTERS:Cataract:0.62), (OXCARBAZEPINE:Cataract:0.29), (OXYBUTYNIN CHLORIDE:Cataract:0.13), (PANTOPRAZOLE MAGNESIUM:Cataract:0.77), (PAZOPANIB HYDROCHLORIDE:Cataract:0.3), (PEMETREXED DISODIUM:Cataract:0.23), (PILSICAINIDE HYDROCHLORIDE:Cataract:0.24), (POVIDONE:Cataract:0.08), (PRAMIPEXOLE:Cataract:0.55), (PRIMIDONE:Cataract:0.29), (PROMETHAZINE HYDROCHLORIDE:Cataract:0.02), (PYRAZINAMIDE:Cataract:0.65), (SACUBITRIL\VALSA RTAN:Cataract:0.66), (SCOPOLAMINE:Cataract:0.4), (SECUKINUMAB:Cataract:0.2), (SOTA LOL:Cataract:0.63), (SULFAMETHOXAZOLE:Cataract:0.17), (TETRACAINE\TETRACAINE HYDROCHLORIDE:Cataract:0.27), (TINZAPARIN SODIUM:Cataract:0.11), (TIOTROPIUM:Cataract:0.1), (TIZANIDINE HYDROCHLORIDE:Cataract:0.18), (TRAMADOL HYDROCHLORIDE:Cataract:0.39), (UMECLIDINIUM BROMIDE\VILANTEROL TRIFENATATE:Cataract:0.13), (VITAMIN B:Cataract:0.37), (CERTOLIZUMAB PEGOL:Cataract operation:0.34), (DEXLANSOPRAZOLE:Cataract operation:0.51), (DILTIAZEM HYDROCHLORIDE:Cataract operation:0.37), (EVOLOCUMAB:Cataract operation:0.52), (FLUTICASONE PROPIONATE:Cataract operation:0.4), (IBRUTINIB:Cataract operation:0.76), (LANSOPRAZOLE:Cataract operation:0.39), (NAPROXEN:Cataract operation:0.59), (OXYGEN:Cataract operation:0.02), (PRAVASTATIN SODIUM:Cataract operation:0.19), (RANOLAZINE:Cataract operation:0.05), (RIBOFLAVIN 5′-PHOSPHATE SODIUM:Cataract operation:0.21), (TOPIRAMATE:Cataract operation:0.32), (TRAZODONE HYDROCHLORIDE:Cataract operation:0.16), (AMINO ACIDS\DEXTROSE\ELECTROLYTES NOS:Catheter management:0.13),(ARMODAFINIL:Catheter management:0.4), (BORTEZOMIB:Catheter management:0.27), (CALCIUM ACETATE:Catheter management:0.3), (CALCIUM CARBONATE\CHOLECALCIFEROL:Catheter management:0.4), (CARVEDILOL PHOSPHATE:Catheter management:0.13), (CERTOLIZUMAB PEGOL:Catheter management:0.5), (CHOLESTYRAMINE:Catheter management:0.74), (DASATINIB:Catheter management:0.45), (DICLOFENAC SODIUM:Catheter management:0.07), (DILTIAZEM:Catheter management:0.28), (ELETRIPTAN HYDROBROMIDE:Catheter management:0.1), (ESZOPICLONE:Catheter management:0.69), (EVEROLIMUS:Catheter management:0.16), (HYDROXYZINE PAMOATE:Catheter management:0.43), (HYOSCYAMINE:Catheter management:0.09), (IBRUTINIB:Catheter management:0.8), (ILOPROST:Catheter management:0.3), (INDOMETHACIN:Catheter management:0.09), (IPRATROPIUM:Catheter management:0.33), (LEVALBUTEROL HYDROCHLORIDE:Catheter management:0.45), (MELOXICAM:Catheter management:0.5), (METHYLPHENIDATE HYDROCHLORIDE:Catheter management:0.05), (NIFEDIPINE:Catheter management:0.01), (PANTOPRAZOLE:Catheter management:0.71), (PHENYTOIN:Catheter management:0.01), (PRAMIPEXOLE DIHYDROCHLORIDE:Catheter management:0.3), (PRAVASTATIN:Catheter management:0.71), (PRIMIDONE:Catheter management:0.4), (RIZATRIPTAN BENZOATE:Catheter management:0.12), (SOTALOL:Catheter management:0.02), (SULFAMETHOXAZOLE:Catheter management:0.2), (TAMSULOSIN:Catheter management:0.77), (TORSEMIDE:Catheter management:0.21), (VARENICLINE TARTRATE:Catheter management:0.41), (VITAMIN B:Catheter management:0.11), (ABIRATERONE ACETATE:Cellulitis:0.1), (ACARBOSE:Cellulitis:0.51), (ACETAMINOPHEN\OXYCODONE\OXYCODONE HYDROCHLORIDE:Cellulitis:0.49), (ACETAMINOPHEN\PROPOXYPHENE NAPSYLATE:Cellulitis:0.27), (ACETAMINOPHEN\TRAMADOL:Cellulitis:0.2), (AGALSIDA SE BETA:Cellulitis:0.02), (ALBIGLUTIDE:Cellulitis:0.24), (ALUMINUM HYDROXIDE\DIMETHICONE\MAGNESIUM HYDROXIDE:Cellulitis:0.27), (AMISULPRIDE:Cellulitis:0.27), (AMLODIPINE\VALSARTA N:Cellulitis:0.21),(ARMODAFINIL:Cellulitis:0.97), (AZELASTINE:Cellulitis:0.21), (BICALUT AMIDE:Cellulitis:0.56), (BIFIDOBACTERIUM LONGUM SUBSP. INFANTIS:Cellulitis:0), (BROMAZEPAM:Cellulitis:0.28), (BUPRENORPHINE HYDROCHLORIDE:Cellulitis:0.87), (BUSPIRONE HYDROCHLORIDE:Cellulitis:1.01), (CALCITONIN SALMON:Cellulitis:0.46), (CALCIUM\MAGNESIUM:Cellulitis:0.1), (CANAGLIFLOZIN:Cellulitis: 0.43), (CANAGLIFLOZIN\METFORMIN HYDROCHLORIDE:Cellulitis:0.45), (CANDESARTAN CILEXETIL\HYDROCHLOROTHIAZIDE:Cellulitis:1.12), (CAPTOPRIL:Cellulitis:0.58), (CA RVEDILOL PHOSPHATE:Cellulitis:1.03), (CHLORPHENIRAMINE\HYDROCODONE:Cellulitis:0.02), (C LONIDINE HYDROCHLORIDE:Cellulitis:0.3), (CLOZAPINE:Cellulitis:1.12), (DACLIZUMAB:Cellulitis:00.65), (DANAZOL:Cellulitis:0.29), (DAPAGLIFLOZIN PROPANEDIOL:Cellulitis:0.34), (DESLORATADINE:Cellulitis:0.87), (DESVENLAFAXINE SUCCINATE:Cellulitis:0.42), (DEXAMETHASONE ACETATE:Cellulitis:0.69), (DEXLANSOPRAZOLE:Cellulitis:1.78), (DIPYRONE:Cellulitis:0.34), (DOCUSATE CALCIUM:Cellulitis:0.49), (DOLASETRON MESYLATE:Cellulitis:0.04), (DOXEPIN:Cellulitis:0.27), (DRONEDARONE:Cellulitis:0.05), (E CONAZOLE:Cellulitis:0.15), (EDOXABAN TOSYLATE:Cellulitis:0.78), (ELETRIPTAN HYDROBROMIDE:Cellulitis:0.87), (ELOTUZUMAB:Cellulitis:0.45), (EMPAGLIFLOZIN\LIN AGLIPTIN:Cellulitis:0.11), (EVOLOCUMAB:Cellulitis:1.36), (FENTANYL CITRATE:Cellulitis:0.08), (FINGOLIMOD HYDROCHLORIDE:Cellulitis:0.67), (FISH OIL\OMEGA-3 FATTY ACIDS:Cellulitis:0.16), (FORMOTEROL FUMARATE:Cellulitis:0.62), (FORMOTEROL FUMARATE\MOMETASONE FUROATE:Cellulitis:0.09), (GIMERACIL\OTERACIL\TEGAFUR:Cellulitis:0.34), (GLYCOPY RROLATE:Cellulitis:0.04), (GLYCOPYRRONIUM\INDACATEROL:Cellulitis:0.6), (HERBAL S\TURMERIC:Cellulitis:0.26), (HYDROCORTISONE ACETATE:Cellulitis:0.16), (HYDROXYCHLOROQUINE DIPHOSPHATE:Cellulitis:0.53), (HYDROXYUREA:Cellulitis:0.35), (IBRITUMOMAB TIUXETAN:Cellulitis:0.25), (ILOPROST:Cellulitis:0.5), (IMIPRAMINE HYDROCHLORIDE:Cellulitis:0.51), (INDAPAMIDE\PERINDOPRIL:Cellulitis:0.65), (INFLIX IMAB-DYYB:Cellulitis:0.04), (INSULIN DEGLUDEC:Cellulitis:0.3), (INSULIN GLULISINE:Cellulitis:0.18), (INTERFERON GAMMA-1B:Cellulitis:0.18), (IRINOTECAN HYDROCHLORIDE:Cellulitis:0.39), (IXAZOMIB:Cellulitis:0.49), (LACTASE:Cellulitis:0.03), (LERCANIDIPINE:Cellulitis:1.03), (LERCANIDIPINE HYDROCHLORIDE:Cellulitis:0.39), (LEVALBUTEROL:Cellulitis:0.07), (LITHIUM CARBONATE:Cellulitis:0.82), (LOMITAPIDE MESYLATE:Cellulitis:0.16), (MEGESTROL:Cellulitis:0.17), (MEGESTROL ACETATE:Cellulitis:0.74), (METFORMIN HYDROCHLORIDE\VILDAGLIPTIN:Cellulitis:0.19), (METHENAMINE HIPPURATE:Cellulitis:0.75), (METHYLPREDNISOLONE ACETATE:Cellulitis:1.25), (MIDODRINE:Cellulitis:0.37), (MIRABEGRON:Cellulitis:0.05), (MI TOXANTRONE HYDROCHLORIDE:Cellulitis:0.18), (NABUMETONE:Cellulitis:0.56), (NALOXONE HYDROCHLORIDE\OXYCODONE HYDROCHLORIDE:Cellulitis:0.85), (NATEGLINIDE:Cellulitis:0.38), (NINTEDANIB:Cellulitis: 0.58), (NORTRIPTYLINE HYDROCHLORIDE:Cellulitis:0.02), (OLODATEROL HYDROCHLORIDE\TIOTROPIUM BROMIDE MONOHYDRATE:Cellulitis:1.09), (OSELTAMIVIR:Cellulitis:0.57), (OXCARBAZEPINE:Cellulitis: 1.67), (PANTOPRAZOLE MAGNESIUM:Cellulitis:1.29), (PEGINTERFERON BETA-1A:Cellulitis:0.48), (PENTOSTATIN:Cellulitis:0.02), (PHENYTOIN SODIUM:Cellulitis:0.19), (PIMECROLIMUS:Cellulitis:0.04), (PIROXICAM:Cellulitis:0.3), (PR EDNISOLONE SODIUM PHOSPHATE:Cellulitis:0.19), (PRIMIDONE:Cellulitis:1.18), (PROPAFENONE HYDROCHLORIDE:Cellulitis:0.08), (PYRAZINAMIDE:Cellulitis:0.74), (REGORAFENIB:Cellulitis: 0.16), (RIVASTIGMINE TARTRATE:Cellulitis:0.03), (RIZATRIPTAN BENZOATE:Cellulitis:0.58), (SACUBITRIL\VALSARTAN:Cellulitis:0.8), (SELEXIPAG:Cellulitis: 0.63), (SEVELAMER HYDROCHLORIDE:Cellulitis:0.05), (SILODOSIN:Cellulitis:0), (SODIUM PHOSPHATE, DIBASIC\SODIUM PHOSPHATE, MONOBASIC:Cellulitis:0), (SUMATRIPTAN SUCCINATE:Cellulitis:0.09), (TERBUTALINE SULFATE:Cellulitis:0.81), (TERIFLUNOMIDE:Cellulitis:0.33), (TETRACYCLINE:Cellulitis:1.16), (THALIDOMIDE:Cellulitis:2.13), (THEOPHYLLINE ANHYDROUS:Cellulitis:0.2), (TOBRAMYCIN:Cellulitis:0.39), (TOCOPHEROL:Cellulitis:0.03), (VARENICLINE TARTRATE:Cellulitis:1.02), (VILAZODONE HYDROCHLORIDE:Cellulitis:0.07), (VITAMIN A:Cellulitis:0.28), (DOCUSATE SODIUM:Cerebral disorder:0.22), (EVOLOCUMAB:Cerebral disorder:0.08), (IBRUTINIB:Cerebral disorder:0.57), (NAPROXEN:Cerebral disorder:0.37), (PANTOPRAZOLE:Cerebral disorder:0.07), (SACUBITRIL\VALSARTAN:Cerebral disorder:0.13), (TAMSULOSIN:Cerebral disorder:0.2), (TERIFLUNOMIDE:Cerebral disorder:0.23), (TRAMADOL:Cerebral disorder:0.52), (ZOLPIDEM\ZOLPIDEM TARTRATE:Cerebral disorder:0.02), (ACENOCOUMAROL:Cerebral infarction:0.36), (ACETAMINOPHEN\CAFFEINE\PROMETHAZINE\SALICYLAMIDE:Cere bral infarction:0.19), (ACETAMINOPHEN\CODEINE PHOSPHATE:Cerebral infarction:0.6), (ADENINE:Cerebral infarction:1.02), (ADO-TRASTUZUMAB EMTANSINE:Cerebral infarction:0.33), (AMOXICILLIN:Cerebral infarction:0.74), (BENIDIPINE:Cerebral infarction:0.67), (BETAMETHASONE BUTYRATE PROPIONATE:Cerebral infarction:0.34), (BETAMETHASONE SODIUM PHOSPHATE\NEOMYCIN SULFATE:Cerebral infarction:0.44), (BETAMETHASONE VALERATE\GENTAMICIN SULFATE:Cerebral infarction:0.19), (BISACODYL OR DOCUSATE SODIUM:Cerebral infarction:0.27), (BIXALOMER:Cerebral infarction:0.35), (BROMAZEPAM:Cerebral infarction:0.41), (BUCOLOME:Cerebral infarction:0.06), (BUFORMIN:Cerebral infarction:0.16), (BUMETANIDE:Cerebral infarction:0.8), (CALCIUM LACTATE:Cerebral infarction:0.71), (CALCIUM\VITAMIN D:Cerebral infarction:0.04), (CAMPHOR\MENTHOL\METHYL SALICYLATE:Cerebral infarction:0.29), (CARBIMAZOLE:Cerebral infarction:0.02), (CEFOTIAM HYDROCHLORIDE:Cerebral infarction:0.01), (CEPHARANTHINE:Cerebral infarction:0.35), (CHIMAPHILA UMBELLATA\MANGANESE CHLORIDE\POPULUS TREMULOIDES\SODIUM TAUROCHOLATE:Cerebral infarction:0.28), (CHLORPHENIRAMINE MALEAT\DIHYDROCODEINE PHOSPHATE\METHYLEPHEDRINE HYDROCHLORIDE, DL-:Cerebral infarction:0.1), (CHOLESTYRAMINE:Cerebral infarction:0.02), (CIFENLINE:Cerebral infarction:0.95), (CILASTATIN SODIUM\IMIPENEM:Cerebral infarction:0.08), (CLENBUTEROL HYDROCHLORIDE:Cerebral infarction:0.4), (CLOPERASTINE:Cerebral infarction:0.27), (CLOTRIMAZOLE:Cerebral infarction:0.18), (CLOZAPINE:Cerebral infarction:1.56), (COLCHICINE:Cerebral infarction:0.47), (CORTISONE\HYDROCORTISONE:Cerebral infarction:0.35), (CYANOCOBALAMIN\LIDOCAINE HYDROCHLORIDE\PYRIDOXINE HYDROCHLORIDE\THIAMINE HYDROCHLORIDE:Cerebral infarction:0.05), (DESLORATADINE:Cerebral infarction:0.27), (DEVICE:Cerebral infarction:0.47), (DIFLUCORTOLONE VALERATE\LIDOCAINE:Cerebral infarction:0.02), (DIMEMORFAN:Cerebral infarction:0.33), (DIMEMORFAN PHOSPHATE:Cerebral infarction:0.1), (DISOPYRAMIDE:Cerebral infarction:0.14), (DOBUTAMINE HYDROCHLORIDE:Cerebral infarction:0.02), (DOCUSATE:Cerebral infarction:0.23), (ECABET:Cerebral infarction:0.36), (ECABET SODIUM:Cerebral infarction:0.58), (ELBASVIR:Cerebral infarction:0.36), (ERGOCALCIFEROL:Cerebral infarction:0.39), (ESOMEPRAZOLE:Cerebral infarction:1.01), (ETORICOXIB:Cerebral infarction:0.6), (ETRETINATE:Cerebral infarction:1.1), (EVEROLIMUS:Cerebral infarction:1.55), (FAROPENEM SODIUM:Cerebral infarction:0.5), (FELBINAC:Cerebral infarction:0.19), (FERRIC HYDROXIDE:Cerebral infarction:0.79), (FERROUS SULFATE\GLYCINE:Cerebral infarction:0.03), (FILGRASTIM:Cerebral infarction:0.46), (FISH OIL:Cerebral infarction:0.17), (FLECAINIDE:Cerebral infarction:0.52), (FLUTICASONE PROPIONATE:Cerebral infarction:0.2), (FULVESTRANT:Cerebral infarction:0.36), (FURSULTIAMINE:Cerebral infarction:1.12), (FURSULTIAMINE HYDROCHLORIDE:Cerebral infarction:0.03), (GLIPIZIDE:Cerebral infarction:0.5), (GLUTAMINE\SODIUM GUALENATE:Cerebral infarction:0.56), (GRAZOPREVIR:Cerebral infarction:0.34), (GUANABENZ ACETATE:Cerebral infarction:1.21), (HYDROCHLOROTHIAZIDE\LISINOPRIL:Cerebral infarction:0.75), (HYDROCODONE:Cerebral infarction:0.02), (IMIDAPRIL HYDROCHLORIDE:Cerebral infarction:0.28), (INDAPAMIDE\PERINDOPRIL:Cerebral infarction:0.24), (IPRATROPIUM:Cerebral infarction:0.01), (ISOSORBIDE:Cerebral infarction:0.53), (ITOPRIDE:Cerebral infarction:0.17), (LANDIOLOL HYDROCHLORIDE:Cerebral infarction:0.26), (LERCANIDIPINE:Cerebral infarction:0.74), (LEVOCABASTINE HYDROCHLORIDE:Cerebral infarction:0.66), (LICORICE ROOT EXTRACT\RHUBARB:Cerebral infarction:0.18), (LULICONAZOLE:Cerebral infarction:0.12), (METHADONE HYDROCHLORIDE:Cerebral infarction:0.27), (MINOCYCLINE\MINOCYCLINE HYDROCHLORIDE:Cerebral infarction:0.24), (MINODRONIC ACID:Cerebral infarction:1.2), (MORPHINE:Cerebral infarction:0.51), (NALFURAFINE HYDROCHLORIDE:Cerebral infarction:0.06), (NAPROXEN:Cerebral infarction:1.26), (NEBIVOLOL HYDROCHLORIDE:Cerebral infarction:0.01), (OMEPRAZOLE MAGNESIUM:Cerebral infarction:0.29), (PEMETREXED:Cerebral infarction:0.16), (PEMETREXED DISODIUM:Cerebral infarction:0.17), (PHENPROCOUMON:Cerebral infarction:0.05), (PIMOBENDAN:Cerebral infarction:1.5), (POLYETHYLENE GLYCOLS:Cerebral infarction:0.66), (POTASSIUM:Cerebral infarction:0.5), (POTASSIUM CHLORIDE\SODIUM CHLORIDE\SODIUM LACTATE:Cerebral infarction:0.4), (POTASSIUM CITRATE\TRISODIUM CITRATE DIHYDRATE:Cerebral infarction:0.98), (PREDNISOLONE SODIUM SUCCINATE:Cerebral infarction:0.39), (RANOLAZINE:Cerebral infarction:1.16), (RILPIVIRINE HYDROCHLORIDE:Cerebral infarction:0.13), (SACUBITRIL\VALSARTAN:Cerebral infarction:1.3), (SARPOGRELATE HYDROCHLORIDE:Cerebral infarction:0.64), (SCOPOLAMINE:Cerebral infarction:0.36), (SECUKINUMAB:Cerebral infarction:0.94), (SIVELESTAT SODIUM:Cerebral infarction:0.02), (SODIUM GUALENATE MONOHYDRATE:Cerebral infarction:0.14), (SOFALCONE:Cerebral infarction:0.01), (SOTALOL:Cerebral infarction:0.73), (SUPLATAST TOSILATE:Cerebral infarction:0.43), (TICAGRELOR:Cerebral infarction:0.42), (TIOTROPIUM:Cerebral infarction:0.1), (TIPEPIDINE HIBENZATE:Cerebral infarction:0.4), (TROXIPIDE:Cerebral infarction:0.31), (ZALTOPROFEN:Cerebral infarction:0.07), (ABACAVIR:Cerebral toxoplasmosis:0.7), (ATAZANAVIR:Cerebral toxoplasmosis:0.52), (ATAZANAVIR\RITONAVIR:Cerebral toxoplasmosis:0.02), (.ALPHA.1-PROTEINASE INHIBITOR HUMAN:Cerebrovascular accident:0.5), (ACETAMINOPHEN\CAFFEINE\OPIUM:Cerebrovascular accident:0.18), (ADO-TRASTUZUMAB EMTANSINE:Cerebrovascular accident:0.81), (ALBIGLUTIDE:Cerebrovascular accident:0.12), (ALISKIREN HEMIFUMARATE:Cerebrovascular accident:0.72), (ALUMINUM HYDROXIDE\DIMETHICONE\MAGNESIUM HYDROXIDE:Cerebrovascular accident:0.86), (AMINO ACIDS\DEXTROSE\ELECTROLYTES NOS:Cerebrovascular accident:0.19), (AMISULPRIDE:Cerebrovascular accident:0.51), (AMLODIPINE\VALSARTAN:Cerebrovascular accident:0.9), (ANTIHISTAMINES NOS:Cerebrovascular accident:0.44), (APREPITANT:Cerebrovascular accident:0.29), (ARIPIPRAZOLE LAUROXIL:Cerebrovascular accident:0.13), (ASUNAPREVIR:Cerebrovascular accident:0.16), (ATROPINE\DIPHENOXYLATE:Cerebrovascular accident:0.1), (BACITRACIN:Cerebrovascular accident:0.33), (BECLOMETHASONE DIPROPIONATE\FORMOTEROL:Cerebrovascular accident:0.01), (BENZTROPINE MESYLATE:Cerebrovascular accident:0.13), (BETAMETHASONE DIPROPIONATE\CALCIPOTRIENE\CALCIPOTRIENE HYDRATE:Cerebrovascular accident:0.7), (BETAMETHASONE\CALCIPOTRIENE:Cerebrovascular accident:0.17), (BOCOCIZUMAB:Cerebrovascular accident:0.09), (BUPRENORPHINE HYDROCHLORIDE:Cerebrovascular accident:1.02), (BUTYLSCOPOLAMINE BROMIDE:Cerebrovascular accident:0.33), (CALCITONIN:Cerebrovascular accident:0.63), (CALCITONIN SALMON:Cerebrovascular accident:0.84), (CALCIUM POLYCARBOPHIL:Cerebrovascular accident:0.24), (CALCIUM\MAGNESIUM\ZINC:Cerebrovascular accident:0.53), (CARBIDOPA:Cerebrovascular accident:0.47), (CASANTHRANOL\DOCUSATE SODIUM:Cerebrovascular accident:0.08), (CEFOTAXIME SODIUM:Cerebrovascular accident:0.21), (CEFPODOXIME PROXETIL:Cerebrovascular accident:0.02), (CETIRIZINE HYDROCHLORIDE\PSEUDOEPHEDRINE HYDROCHLORIDE:Cerebrovascular accident:0.54), (CETUXIMAB:Cerebrovascular accident:0.34), (CHLORDIAZEPOXIDE HYDROCHLORIDE\CLIDINIUM BROMIDE:Cerebrovascular accident:0.44), (CHLORHEXIDINE:Cerebrovascular accident:0.41), (CHLORPHENIRAMINE\HYDROCODONE:Cerebrovascular accident:0.03), (CHLORPROMAZINE:Cerebrovascular accident:0.31), (CICLESONIDE:Cerebrovascular accident:0.94), (CILASTATIN SODIUM\IMIPENEM:Cerebrovascular accident:0.7), (CIPROFIBRATE:Cerebrovascular accident:0.27), (CLOMIPRAMINE HYDROCHLORIDE:Cerebrovascular accident:0.34), (COD LIVER OIL:Cerebrovascular accident:0.51), (CODEINE PHOSPHATE:Cerebrovascular accident:0.23), (DABRAFENIB:Cerebrovascular accident:0.15), (DACLIZUMAB:Cerebrovascular accident:1.37), (DANTROLENE SODIUM:Cerebrovascular accident:0.17), (DEFLAZACORT:Cerebrovascular accident:1.05), (DESONIDE:Cerebrovascular accident:0.69), (DEXTROAMPHETAMINE SULFATE:Cerebrovascular accident:0.28), (DIOSMIN:Cerebrovascular accident:0.09), (DOLASETRON MESYLATE:Cerebrovascular accident:0.13), (DOXYLAMINE SUCCINATE:Cerebrovascular accident:0.22), (DRONABINOL:Cerebrovascular accident:1.29), (ELOTUZUMAB:Cerebrovascular accident:0.1), (EMPAGLIFLOZIN\METFORMIN HYDROCHLORIDE:Cerebrovascular accident:0.16), (EPIRUBICIN:Cerebrovascular accident:0.36), (FORMOTEROL FUMARATE:Cerebrovascular accident:1.49), (FORMOTEROL FUMARATE\MOMETASONE FUROATE:Cerebrovascular accident:0.55), (GARLIC:Cerebrovascular accident:0.32), (GLIMEPIRIDE\ROSIGLITAZONE MALEATE:Cerebrovascular accident:0.17), (GLYCOPYRROLATE\INDACATEROL MALEATE:Cerebrovascular accident:0.38), (GLYCOPYRRONIUM:Cerebrovascular accident:0.32), (GUANFACINE HYDROCHLORIDE:Cerebrovascular accident:0.05), (HYALURONATE SODIUM:Cerebrovascular accident:0.17), (HYDROCODONE BITARTRATE\IBUPROFEN:Cerebrovascular accident:0.47), (HYDROCORTISONE ACETATE:Cerebrovascular accident:0.28), (HYDROXYCHLOROQUINE DIPHOSPHATE:Cerebrovascular accident:0.57), (ILOPROST:Cerebrovascular accident:0.6), (IMIGLUCERASE:Cerebrovascular accident:0), (INFLIXIMAB-DYYB:Cerebrovascular accident:0.29), (INSULIN DEGLUDEC:Cerebrovascular accident:0.82), (INSULIN PORK\ANSULIN PURIFIED PORK:Cerebrovascular accident:0.12), (INTERFERON:Cerebrovascular accident:0.4), (INTERFERON ALFA-2A:Cerebrovascular accident:0.85), (IODINE:Cerebrovascular accident:0.25), (IRINOTECAN HYDROCHLORIDE:Cerebrovascular accident:0.31), (IRON SUCROSE:Cerebrovascular accident:0.24), (IXAZOMIB:Cerebrovascular accident:0.3), (LAPATINIB DITOSYLATE:Cerebrovascular accident:0.21), (LEVALBUTEROL HYDROCHLORIDE:Cerebrovascular accident:1.53), (LIDOCAINE\PRILOCAINE:Cerebrovascular accident:0.7), (LIOTHYRONINE:Cerebrovascular accident:0.44), (LISDEXAMFETAMINE DIMESYLATE:Cerebrovascular accident:0.89), (LURASIDONE HYDROCHLORIDE:Cerebrovascular accident:0.29), (LUTETIUM LU-177:Cerebrovascular accident:0), (MEDROXYPROGESTERONE ACETATE:Cerebrovascular accident:0.68), (MEFENAMIC ACID:Cerebrovascular accident:0.23), (MEGESTROL:Cerebrovascular accident:0.05), (METFORMIN HYDROCHLORIDE\ROSIGLITAZONE MALEATE:Cerebrovascular accident:0.5), (METFORMIN HYDROCHLORIDE\SAXAGLIPTIN:Cerebrovascular accident:0.09), (METFORMIN\SITAGLIPTIN:Cerebrovascular accident:0.05), (METRELEPTIN:Cerebrovascular accident:0.47), (MOXIFLOXACIN:Cerebrovascular accident:0.62), (NALOXONE:Cerebrovascular accident:0.16), (NIRAPARIB:Cerebrovascular accident:0.4), (ORPHENADRINE:Cerebrovascular accident:0.56), (OSELTAMIVIR:Cerebrovascular accident:0.15), (OSELTAMIVIR PHOSPHATE:Cerebrovascular accident:0.34), (OXYBUTYNIN\OXYBUTYNIN CHLORIDE:Cerebrovascular accident:0.3), (PANITUMUMAB:Cerebrovascular accident:0.14), (PARICALCITOL:Cerebrovascular accident:0.68), (PEGINTERFERON BETA-1A:Cerebrovascular accident:1.29), (PEMETREXED:Cerebrovascular accident:0.23), (PEMETREXED DISODIUM:Cerebrovascular accident:0.81), (PENICILLIN G:Cerebrovascular accident:0.06), (PIMECROLIMUS:Cerebrovascular accident:0.11), (PRASUGREL:Cerebrovascular accident:0.2), (PRAZOSIN HYDROCHLORIDE:Cerebrovascular accident:0.72), (PREDNISOLONE\PREDNISOLONE ACETATE\PREDNISOLONE SODIUM SUCCINATE:Cerebrovascular accident:0.61), (PRISTINAMYCIN:Cerebrovascular accident:0.24), (RADIATION THERAPY:Cerebrovascular accident:0.03), (RALTEGRAVIR POTASSIUM:Cerebrovascular accident:0.31), (RETINOL:Cerebrovascular accident:0.32), (RIZATRIPTAN:Cerebrovascular accident:0.38), (RIZATRIPTAN BENZOATE:Cerebrovascular accident:1.32), (SELEGILINE:Cerebrovascular accident:0.21), (SELEGILINE HYDROCHLORIDE:Cerebrovascular accident:0.14), (SEVELAMER HYDROCHLORIDE:Cerebrovascular accident:0.31), (SILVER SULFADIAZINE:Cerebrovascular accident:0.43), (SOFOSBUVIR\VELPATASVIR:Cerebrovascular accident:0.03), (SOLIFENACIN:Cerebrovascular accident:0.01), (STARCH, WHEAT:Cerebrovascular accident:0.04), (SULFAMETHOXAZOLE:Cerebrovascular accident:0.96), (TAFLUPROST:Cerebrovascular accident:0.07), (TEDUGLUTIDE\WATER:Cerebrovascular accident:0.04), (TELOTRISTAT ETHYL:Cerebrovascular accident:0.04), (TESTOSTERONE UNDECANOATE:Cerebrovascular accident:0.91), (TETRACYCLINE:Cerebrovascular accident:1.34), (THIOCTIC ACID:Cerebrovascular accident:0.3), (TIMOLOL MALEATE\TRAVOPROST:Cerebrovascular accident:0.26), (TIMOLOL\TRAVOPROST:Cerebrovascular accident:0.16), (TINZAPARIN:Cerebrovascular accident:0.69), (TIPIRACIL HYDROCHLORIDE\TRIFLURIDINE:Cerebrovascular accident:0.01), (TRIPTORELIN PAMOATE:Cerebrovascular accident:0.17), (UMECLIDINIUM BROMIDE:Cerebrovascular accident:0.71), (VANCOMYCIN HYDROCHLORIDE:Cerebrovascular accident:0.22), (VARDENAFIL HYDROCHLORIDE TRIHYDRATE:Cerebrovascular accident:0.25), (YEAST:Cerebrovascular accident:0.19), (ZILEUTON:Cerebrovascular accident:0.12), (ZOLMITRIPTAN:Cerebrovascular accident:0.61), (ZONISAMIDE:Cerebrovascular accident:0.51), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Cerebrovascular accident prophylaxis:0.64), (.ALPHA.1-PROTEINASE INHIBITOR HUMAN:Cerebrovascular accident prophylaxis:0.59), (ABACAVIR SULFATE\DOLUTEGRAVIR SODIUM\LAMIVUDINE:Cerebrovascular accident prophylaxis:0.51), (ABOBOTULINUMTOXINA:Cerebrovascular accident prophylaxis:0.35), (ACAMPROSATE:Cerebrovascular accident prophylaxis:0.23), (ACETAMINOPHEN\APRONALIDE\CAFFEINE\PROPYPHENAZONE:Ce rebrovascular accident prophylaxis:0.7), (ACETAMINOPHEN\CAFFEINE\PROMETHAZINE\SALICYLAMIDE:Cere brovascular accident prophylaxis:0.34), (ACETAMINOPHEN\DICHLORALPHENAZONE\ISOMETHEPTENE:Cere brovascular accident prophylaxis:0.05), (ACETAMINOPHEN\METHOCARBAMOL:Cerebrovascular accident prophylaxis:0.07), (ACETAMINOPHEN\PROPOXYPHENE HYDROCHLORIDE:Cerebrovascular accident prophylaxis:1.07), (ADO-TRASTUZUMAB EMTANSINE:Cerebrovascular accident prophylaxis:1.54), (ADUCANUMAB:Cerebrovascular accident prophylaxis:0.07), (AGALSIDASE BETA:Cerebrovascular accident prophylaxis:1.99), (ALEMTUZUMAB:Cerebrovascular accident prophylaxis:0.71), (ALUMINUM HYDROXIDE\MAGNESIUM HYDROXIDE\SIMETHICONE:Cerebrovascular accident prophylaxis:0.37), (ALUMINUM HYDROXIDE\MAGNESIUM OXIDE\OXETHAZAINE:Cerebrovascular accident prophylaxis:0.05), (ALUMINUM SILICATE:Cerebrovascular accident prophylaxis:0.27), (AMINO ACIDS\CARBOHYDRATES\ELECTROLYTES NOS\VITAMINS:Cerebrovascular accident prophylaxis:0.07), (AMINO ACIDS\CARBOHYDRATES\MINERALS\SOYBEAN OIL\VITAMINS:Cerebrovascular accident prophylaxis:0.2), (AMINO ACIDS\DEXTROSE\ELECTROLYTES NOS:Cerebrovascular accident prophylaxis:1.27), (AMOXICILLIN\CLARITHROMYCIN\ESOMEPRAZOLE:Cerebrovascular accident prophylaxis:1.12), (AMPICILLIN SODIUM:Cerebrovascular accident prophylaxis:0.65), (AMYLASE\LIPASE\PROTEASE:Cerebrovascular accident prophylaxis:0.02), (ANAGRELIDE HYDROCHLORIDE:Cerebrovascular accident prophylaxis:1.25), (ANHYDROUS CITRIC ACID\ASPIRIN\SODIUM BICARBONATE:Cerebrovascular accident prophylaxis:0.38), (ASCORBIC ACID\BIOTIN\CALCIUM PANTOTHENATE\CYANOCOBALAMIN\FOLIC ACID\NIACINAMIDE\PYRIDOXINE HYDROCHLORIDE\RIBOFLAVIN\THIAMINE MONONITRATE:Cerebrovascular accident prophylaxis:0.21), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Cerebrovascular accident prophylaxis:0.48), (ASCORBIC ACID\FERROUS SULFATE:Cerebrovascular accident prophylaxis:0.03), (ASIAN GINSENG:Cerebrovascular accident prophylaxis:0.16), (ATOVAQUONE:Cerebrovascular accident prophylaxis:0.31), (AZITHROMYCIN MONOHYDRATE:Cerebrovascular accident prophylaxis:0.38), (BAMIFYLLINE HYDROCHLORIDE:Cerebrovascular accident prophylaxis:0.56), (BENDAMUSTINE HYDROCHLORIDE:Cerebrovascular accident prophylaxis:0.12), (BENZOCAINE:Cerebrovascular accident prophylaxis:0.31), (BERAPROST:Cerebrovascular accident prophylaxis:0.27), (BETAMETHASONE BUTYRATE PROPIONATE:Cerebrovascular accident prophylaxis:0.49), (BETAMETHASONE SODIUM PHOSPHATE\NEOMYCIN SULFATE:Cerebrovascular accident prophylaxis:0.17), (BETAMETHASONE\CALCIPOTRIENE:Cerebrovascular accident prophylaxis:1.08), (BETAMETHASONE\DEXCHLORPHENIRAMINE MALEATE:Cerebrovascular accident prophylaxis:0.14), (BIPERIDEN:Cerebrovascular accident prophylaxis:0.14), (BIXALOMER:Cerebrovascular accident prophylaxis:0.32), (BLACK COHOSH:Cerebrovascular accident prophylaxis:0.01), (BLONANSERIN:Cerebrovascular accident prophylaxis:0.35), (BOCEPREVIR:Cerebrovascular accident prophylaxis:0.45), (BOSUTINIB:Cerebrovascular accident prophylaxis:0.13), (BROMHEXINE:Cerebrovascular accident prophylaxis:0.25), (BUFORMIN:Cerebrovascular accident prophylaxis:0.39), (CABAZITAXEL:Cerebrovascular accident prophylaxis:0.6), (CABOZANTINIB:Cerebrovascular accident prophylaxis:0.14), (CABOZANTINIB S-MALATE:Cerebrovascular accident prophylaxis:0.33), (CAJUPUT OIL\EUCALYPTUS OIL\PEPPERMINT OIL:Cerebrovascular accident prophylaxis:0.07), (CALCIUM CARBONATE\ETIDRONATE DISODIUM:Cerebrovascular accident prophylaxis:0.09), (CALCIUM CHLORIDE\DEXTROSE\POTASSIUM CHLORIDE\SODIUM ACETATE\SODIUM CHLORIDE:Cerebrovascular accident prophylaxis:0.28), (CALCIUM PANTOTHENATE\CYANOCOBALAMIN\NIACINAMIDE\PYRIDOXINE HYDROCHLORIDE\RIBOFLAVIN\THIAMINE MONONITRATE:Cerebrovascular accident prophylaxis:0.33), (CALCIUM\VITAMINS:Cerebrovascular accident prophylaxis:0.07), (CASPOFUNGIN:Cerebrovascular accident prophylaxis:0.14), (CERITINIB:Cerebrovascular accident prophylaxis:0.8), (CERIVASTATIN SODIUM:Cerebrovascular accident prophylaxis:0.89), (CEVIMELINE HYDROCHLORIDE:Cerebrovascular accident prophylaxis:0.65), (CHOLINE BITARTRATE\CYSTEINE\INOSITOL\MAMMAL LIVER:Cerebrovascular accident prophylaxis:0.03), (CLOPERASTINE:Cerebrovascular accident prophylaxis:0.15), (CLOXACILLIN SODIUM:Cerebrovascular accident prophylaxis:0.44), (COLCHICINE\DICYCLOMINE HYDROCHLORIDE:Cerebrovascular accident prophylaxis:0.58), (COLISTIMETHATE SODIUM:Cerebrovascular accident prophylaxis:0.12), (CONVALLARIA MAJALIS\CRATAEGUS LAEVIGATA LEAF\PROXYPHYLLINE:Cerebrovascular accident prophylaxis:0.23), (CORTICOTROPIN:Cerebrovascular accident prophylaxis:1.16), (CROTAMITON:Cerebrovascular accident prophylaxis:0.2), (CYPROTERONE:Cerebrovascular accident prophylaxis:0.07), (DABRAFENIB:Cerebrovascular accident prophylaxis:0.71), (DABRAFENIB MESYLATE:Cerebrovascular accident prophylaxis:0.12), (DACLIZUMAB:Cerebrovascular accident prophylaxis:1.47), (DANAZOL:Cerebrovascular accident prophylaxis:1.53), (DAPAGLIFLOZIN PROPANEDIOL\METFORMIN HYDROCHLORIDE:Cerebrovascular accident prophylaxis:0.65), (DASABUVIR\OMBITASVIR\PARITAPREVIR\RITONAVIR:Cerebrovasc ular accident prophylaxis:0.56), (DEGARELIX ACETATE:Cerebrovascular accident prophylaxis:0.15), (DESONIDE:Cerebrovascular accident prophylaxis:1.48), (DESVENLAFAXINE:Cerebrovascular accident prophylaxis:0.1), (DEXTRAN:Cerebrovascular accident prophylaxis:0.17), (DEXTROAMPHETAMINE:Cerebrovascular accident prophylaxis:0.38), (DEXTROAMPHETAMINE SULFATE:Cerebrovascular accident prophylaxis:0.96), (DEXTROMETHORPHAN HYDROBROMIDE\QUINIDINE SULFATE:Cerebrovascular accident prophylaxis:0.07), (DIAZOXIDE:Cerebrovascular accident prophylaxis:0.13), (DIPHENHYDRAMINE HYDROCHLORIDE\LIDOCAINE HYDROCHLORIDE\NYSTATIN:Cerebrovascular accident prophylaxis:0.1), (DIPYRONE:Cerebrovascular accident prophylaxis:0.62), (DIPYRONE\PROMETHAZINE HYDROCHLORIDE:Cerebrovascular accident prophylaxis:0.61), (DOBUTAMINE HYDROCHLORIDE:Cerebrovascular accident prophylaxis:1.5), (DOLASETRON MESYLATE:Cerebrovascular accident prophylaxis:0.86), (DOXYLAMINE SUCCINATE:Cerebrovascular accident prophylaxis:0.58), (ECABET SODIUM:Cerebrovascular accident prophylaxis:0.66), (ECONAZOLE:Cerebrovascular accident prophylaxis:2.64), (EFINACONAZOLE:Cerebrovascular accident prophylaxis:0.22), (ELOTUZUMAB:Cerebrovascular accident prophylaxis:0.88), (EMPAGLIFLOZIN\METFORMIN HYDROCHLORIDE:Cerebrovascular accident prophylaxis:0.17), (EPIRUBICIN:Cerebrovascular accident prophylaxis:0.6), (ERGOLOID MESYLATES:Cerebrovascular accident prophylaxis:0.04), (ERIBULIN MESYLATE:Cerebrovascular accident prophylaxis:0.03), (ERYTHROMYCIN\SULFISOXAZOLE:Cerebrovascular accident prophylaxis:0.63), (ESTROGENS, ESTERIFIED\METHYLTESTOSTERONE:Cerebrovascular accident prophylaxis:0.13), (ETHINYL ESTRADIOL\NORGESTIMATE:Cerebrovascular accident prophylaxis:0.09), (ETHINYL ESTRADIOL\NORGESTREL:Cerebrovascular accident prophylaxis:0.23), (ETRETINATE:Cerebrovascular accident prophylaxis:1.46), (EUCALYPTOL\MENTHOL\METHYL SALICYLATE\THYMOL:Cerebrovascular accident prophylaxis:0.08), (FAROPENEM SODIUM:Cerebrovascular accident prophylaxis:0.41), (FERRIC CITRATE ANHYDROUS:Cerebrovascular accident prophylaxis:1.11), (FERRIC HYDROXIDE:Cerebrovascular accident prophylaxis:1.17), (FERRIC OXYHYDROXIDE:Cerebrovascular accident prophylaxis:0.58), (FERROUS BISGLYCINATE:Cerebrovascular accident prophylaxis:0.81), (FLAVIN ADENINE DINUCLEOTIDE:Cerebrovascular accident prophylaxis:0.88), (FLUNARIZINE HYDROCHLORIDE:Cerebrovascular accident prophylaxis:0.51), (FLUPHENAZINE:Cerebrovascular accident prophylaxis:0.07), (FLUPIRTINE:Cerebrovascular accident prophylaxis:0.06), (FLURAZEPAM\FLURAZEPAM HYDROCHLORIDE:Cerebrovascular accident prophylaxis:0.4), (FORMOTEROL FUMARATE\MOMETASONE FUROATE:Cerebrovascular accident prophylaxis:1.2), (FOSFOMYCIN CALCIUM:Cerebrovascular accident prophylaxis:0.19), (GLIMEPIRIDE\ROSIGLITAZONE MALEATE:Cerebrovascular accident prophylaxis:0.92), (GLYCOPYRROLATE\INDACATEROL MALEATE:Cerebrovascular accident prophylaxis:1.19), (HERBALS\KARAYA GUM:Cerebrovascular accident prophylaxis:0.05), (HORSE CHESTNUT:Cerebrovascular accident prophylaxis:0.17), (IMIGLUCERASE:Cerebrovascular accident prophylaxis:1.01), (IMIPENEM:Cerebrovascular accident prophylaxis:0.22), (IMMUNE GLOBULIN NOS:Cerebrovascular accident prophylaxis:0.47), (INFLIXIMAB-DYYB:Cerebrovascular accident prophylaxis:1.17), (INSULIN PORK\INSULIN PURIFIED PORK:Cerebrovascular accident prophylaxis:0.57), (INTERFERON ALFA-2A:Cerebrovascular accident prophylaxis:1.09), (INTERFERON ALFA-2B:Cerebrovascular accident prophylaxis:0.39), (INTERFERON GAMMA-1B:Cerebrovascular accident prophylaxis:0.12), (IPRAGLIFLOZIN:Cerebrovascular accident prophylaxis:0.34), (ISONIAZID\PYRAZINAMIDE\RIFAMPIN:Cerebrovascular accident prophylaxis:0.4), (ITRACONAZOLE\SODIUM CHLORIDE:Cerebrovascular accident prophylaxis:0.04), (IXAZOMIB:Cerebrovascular accident prophylaxis:1.15), (LANREOTIDE:Cerebrovascular accident prophylaxis:0.61), (LAPATINIB DITOSYLATE:Cerebrovascular accident prophylaxis:0.74), (LENOGRASTIM:Cerebrovascular accident prophylaxis:0.92), (LEVOCARNITINE HYDROCHLORIDE:Cerebrovascular accident prophylaxis:0.76), (LEVOMILNACIPRAN HYDROCHLORIDE:Cerebrovascular accident prophylaxis:0.24), (LEVOSIMENDAN:Cerebrovascular accident prophylaxis:0.95), (LINSIDOMINE:Cerebrovascular accident prophylaxis:0.98), (LISDEXAMFETAMINE DIMESYLATE:Cerebrovascular accident prophylaxis:1.23), (LITHIUM:Cerebrovascular accident prophylaxis:1.19), (LOMITAPIDE MESYLATE:Cerebrovascular accident prophylaxis:1.35), (LUTETIUM LU-177:Cerebrovascular accident prophylaxis:0.47), (MAGALDRATE:Cerebrovascular accident prophylaxis:0.34), (MAGNESIUM PIDOLATE:Cerebrovascular accident prophylaxis:0.15), (MAGNESIUM\PYRIDOXINE:Cerebrovascular accident prophylaxis:0.01), (MAXACALCITOL:Cerebrovascular accident prophylaxis:1.31), (MENTHOL\ZINC OXIDE:Cerebrovascular accident prophylaxis:0.19), (MEPOLIZUMAB:Cerebrovascular accident prophylaxis:1.07), (METFORMIN HYDROCHLORIDE\ROSIGLITAZONE MALEATE:Cerebrovascular accident prophylaxis:0.96), (METFORMIN HYDROCHLORIDE\SAXAGLIPTIN:Cerebrovascular accident prophylaxis:0.83), (METHENAMINE MANDELATE:Cerebrovascular accident prophylaxis:0.24), (METHENOLONE ENANTHATE:Cerebrovascular accident prophylaxis:1.2), (METHOXY POLYETHYLENE GLYCOL-EPOETIN BETA:Cerebrovascular accident prophylaxis:1.19), (MILNACIPRAN:Cerebrovascular accident prophylaxis:0.15), (MOEXIPRIL HYDROCHLORIDE:Cerebrovascular accident prophylaxis:1.61), (MORPHINE HYDROCHLORIDE:Cerebrovascular accident prophylaxis:0.66), (MORPHINE SULFATE\NALTREXONE HYDROCHLORIDE:Cerebrovascular accident prophylaxis:0.8), (MOSAPRIDE CITRATE DIHYDRATE:Cerebrovascular accident prophylaxis:0.25), (NALOXEGOL OXALATE:Cerebrovascular accident prophylaxis:0.26), (NARATRIPTAN HYDROCHLORIDE:Cerebrovascular accident prophylaxis:0.32), (NEFAZODONE HYDROCHLORIDE:Cerebrovascular accident prophylaxis:0.22), (NIAOULI OIL\QUININE BENZOATE\THIAMINE HYDROCHLORIDE:Cerebrovascular accident prophylaxis:0.01), (NIRAPARIB:Cerebrovascular accident prophylaxis:0.93), (OMEGA-3-CARBOXYLIC ACIDS:Cerebrovascular accident prophylaxis:0.95), (OSELTAMIVIR:Cerebrovascular accident prophylaxis:1.44), (OSPEMIFENE:Cerebrovascular accident prophylaxis:0.7), (PALIPERIDONE PALMITATE:Cerebrovascular accident prophylaxis:0.48), (PARATHYROID HORMONE:Cerebrovascular accident prophylaxis:0.03), (PASIREOTIDE:Cerebrovascular accident prophylaxis:0.77), (PATIROMER:Cerebrovascular accident prophylaxis:1.17), (PAZOPANIB:Cerebrovascular accident prophylaxis:0.12), (PEGINTERFERON BETA-1A:Cerebrovascular accident prophylaxis:1.1), (PEGZEREPOETIN ALFA:Cerebrovascular accident prophylaxis:1.2), (PENTAMIDINE ISETHIONATE:Cerebrovascular accident prophylaxis:0.44), (PERTUZUMAB:Cerebrovascular accident prophylaxis:0.42), (PHENAZOPYRIDINE HYDROCHLORIDE:Cerebrovascular accident prophylaxis:0.67), (PHENOTHIAZINE:Cerebrovascular accident prophylaxis:0.12), (PIRENZEPINE:Cerebrovascular accident prophylaxis:0.08), (PITOFENONE:Cerebrovascular accident prophylaxis:0.07), (POLYMYXIN B SULFATE\TRIMETHOPRIM SULFATE:Cerebrovascular accident prophylaxis:0.07), (POLYSTYRENE:Cerebrovascular accident prophylaxis:1.01), (POLYSTYRENE SULFONIC ACID:Cerebrovascular accident prophylaxis:0.45), (PORFIMER SODIUM:Cerebrovascular accident prophylaxis:0.08), (POTASSIUM CHLORIDE\SODIUM CHLORIDE\SODIUM LACTATE:Cerebrovascular accident prophylaxis:0.38), (POTASSIUM PHOSPHATE, MONOBASIC:Cerebrovascular accident prophylaxis:0.3), (POVIDONE:Cerebrovascular accident prophylaxis:0.8), (PRAMLINTIDE ACETATE:Cerebrovascular accident prophylaxis:0.15), (PRAMOXINE HYDROCHLORIDE:Cerebrovascular accident prophylaxis:0.08), (PREDNISOLONE SODIUM PHOSPHATE:Cerebrovascular accident prophylaxis:0.01), (PREDNISOLONE VALERATE ACETATE:Cerebrovascular accident prophylaxis:0.04), (PROBUCOL:Cerebrovascular accident prophylaxis:1.87), (PROTRIPTYLINE HYDROCHLORIDE:Cerebrovascular accident prophylaxis:0.83), (PRUCALOPRIDE SUCCINATE:Cerebrovascular accident prophylaxis:0.61), (PYRIDOXAL PHOSPHATE:Cerebrovascular accident prophylaxis:0.02), (QUINACRINE:Cerebrovascular accident prophylaxis:0.55), (RADIATION THERAPY:Cerebrovascular accident prophylaxis:0.86), (RALTEGRAVIR POTASSIUM:Cerebrovascular accident prophylaxis:1.26), (RAMUCIRUMAB:Cerebrovascular accident prophylaxis:0.94), (REGORAFENIB:Cerebrovascular accident prophylaxis:1.62), (RIFABUTIN:Cerebrovascular accident prophylaxis:0.02), (RISEDRONIC ACID:Cerebrovascular accident prophylaxis:0.48), (ROSIGLITAZONE:Cerebrovascular accident prophylaxis:0.34), (RUPATADINE FUMARATE:Cerebrovascular accident prophylaxis:0.04), (SACUBITRIL:Cerebrovascular accident prophylaxis:0.05), (SARPOGRELATE HYDROCHLORIDE:Cerebrovascular accident prophylaxis:0.88), (SECUKINUMAB:Cerebrovascular accident prophylaxis:1.4), (SELEXIPAG:Cerebrovascular accident prophylaxis:0.79), (SENNOSIDE B:Cerebrovascular accident prophylaxis:0.21), (SIBUTRAMINE HYDROCHLORIDE:Cerebrovascular accident prophylaxis:0.53), (SIPULEUCEL-T:Cerebrovascular accident prophylaxis:0.3), (SITAFLOXACIN:Cerebrovascular accident prophylaxis:0.3), (SODIUM GUALENATE MONOHYDRATE:Cerebrovascular accident prophylaxis:0.14), (SODIUM OXYBATE:Cerebrovascular accident prophylaxis:1.66), (SOFOSBUVIR\VELPATASVIR:Cerebrovascular accident prophylaxis:0.62), (SULTOPRIDE HYDROCHLORIDE:Cerebrovascular accident prophylaxis:0.18), (SUPLATAST TOSILATE:Cerebrovascular accident prophylaxis:1.02), (TANDOSPIRONE:Cerebrovascular accident prophylaxis:0.12), (TEDUGLUTIDE\WATER:Cerebrovascular accident prophylaxis:0.71), (TEMSIROLIMUS:Cerebrovascular accident prophylaxis:0.38), (TESTOSTERONE UNDECANOATE:Cerebrovascular accident prophylaxis:1.82), (TIBOLONE:Cerebrovascular accident prophylaxis:0.5), (TIMOLOL\TRAVOPROST:Cerebrovascular accident prophylaxis:0.96), (TIPIRACIL HYDROCHLORIDE\TRIFLURIDINE:Cerebrovascular accident prophylaxis:0.28), (TRAMETINIB:Cerebrovascular accident prophylaxis:0.69), (TREPROSTINIL DIOLAMINE:Cerebrovascular accident prophylaxis:0.26), (TRIHEXYPHENIDYL:Cerebrovascular accident prophylaxis:0.82), (TRIPTORELIN PAMOATE:Cerebrovascular accident prophylaxis:0.72), (UROKINASE:Cerebrovascular accident prophylaxis:1.01), (VALDECOXIB:Cerebrovascular accident prophylaxis:0.92), (VINORELBINE TARTRATE:Cerebrovascular accident prophylaxis:0.57), (VISMODEGIB:Cerebrovascular accident prophylaxis:0.12), (VORAPAXAR SULFATE:Cerebrovascular accident prophylaxis:0.63), (ZICONOTIDE ACETATE:Cerebrovascular accident prophylaxis:0.04), (ZILEUTON:Cerebrovascular accident prophylaxis:0.9), (ZIV-AFLIBERCEPT:Cerebrovascular accident prophylaxis:0.37), (ZOTEPINE:Cerebrovascular accident prophylaxis:0.08), (LOVASTATIN:Cerebrovascular disorder:0.02), (PANTOPRAZOLE:Cerebrovascular disorder:0.08), (RANOLAZINE:Cerebrovascular disorder:0.02), (SACUBITRIL\VALSARTAN:Cerebrovascular disorder:0.08), (.ALPHA.1-PROTEINASE INHIBITOR HUMAN:Chemotherapy:0.04), (ACARBOSE:Chemotherapy:0.36), (ACETAMINOPHEN\PROP OXYPHENE HYDROCHLORIDE:Chemotherapy:0.16), (ACETAMINOPHEN\PROPOXYPHENE NAPSYLATE:Chemotherapy:0.35), (ACTIVATED CHARCOAL:Chemotherapy:0.04), (AGALSIDASE BETA:Chemotherapy:0.41), (ALBUTEROL\IPRATROPIUM:Chemotherapy:0.08), (ALECTINI B:Chemotherapy:0.12), (AMINO ACIDS\DEXTROSE\ELECTROLYTES NOS:Chemotherapy:0.46), (AMISULPRIDE:Chemotherapy:0.94), (AMMONIUM LACTATE:Chemotherapy:0.21), (ANIDULAFUNGIN:Chemotherapy:0.47), (ARSENIC TRIOXIDE:Chemotherapy:0.71), (AVELUMAB:Chemotherapy:0.04), (AZILSARTAN KAMEDOXOMIL:Chemotherapy:0.18), (BACITRACIN:Chemotherapy:0.19), (BELATACEPT: Chemotherapy:0.31), (BENAZEPRIL\BENAZEPRIL HYDROCHLORIDE:Chemotherapy:0.79), (BETAMETHASONE DIPROPIONATE\CLOTRIMAZOLE:Chemotherapy:0.04), (BETAMETHASONE\CALCIPOT RIENE:Chemotherapy:0.07), (BICARBONATE ION:Chemotherapy:0.14), (BOCEPREVIR:Chemotherapy:0.14), (BRINCIDOFOVIR:Chemotherapy: 0.05), (BUDESONIDE\FORMOTEROL FUMARATE:Chemotherapy:0.53), (BUPRENORPHINE HYDROCHLORIDE\NALOXONE HYDROCHLORIDE:Chemotherapy:0.72), (CABOZANTINIB:Chemotherapy:0.14), (CABOZA NTINIB S-MALATE:Chemotherapy:0.2), (CALCIPOTRIENE:Chemotherapy:0.02), (CALCITONIN SALMON:Chemotherapy:1.13), (CALCIUM ACETATE:Chemotherapy:1.05), (CALCIUM\MAGNESIUM:Chemotherapy:0.17), (CANAGLIF LOZIN:Chemotherapy:0.23), (CANAGLIFLOZIN\METFORMIN HYDROCHLORIDE:Chemotherapy:0.63), (CANAKINUMAB:Chemotherapy:0.66), (CARVEDI LOL PHOSPHATE:Chemotherapy:0.76), (CEFPODOXIME PROXETIL:Chemotherapy:0.18), (CERITINIB:Chemotherapy:1.53), (CLOMIPRAMINE HYDROCHLORIDE:Chemotherapy:0.41), (DANAZOL:Chemotherapy:0.95), (DAPAGLIFLOZIN PROPANEDIOL:Chemotherapy:0.07), (DIGITOXIN:Chemotherapy:0.83), (DIPHENHYDRAMINE HYDROCHLORIDE\LIDOCAINE HYDROCHLORIDE\NYSTATIN:Chemotherapy:0.32), (DOLASETRON MESYLATE:Chemotherapy:0.82), (DORIPENEM:Chemotherapy:0.22), (DOXEPIN HYDROCHLORIDE:Chemotherapy:0.26), (DURVALUMAB:Chemotherapy:0.38), (ECONAZO LE:Chemotherapy:0.81), (ELETRIPTAN HYDROBROMIDE:Chemotherapy:1.06), (ELTROMBOPAG OLAMINE:Chemotherapy:0.15), (EMPAGLIFLOZIN\LINAGLIPTIN:Chemotherapy:0.18), (EM TRICITABINE\TENOFOVIR:Chemotherapy:0.05), (EPLERENONE:Chemotherapy:0.51), (ERY THROMYCIN:Chemotherapy:1.04), (ESTROGENS, CONJUGATED\MEDROXYPROGESTERONE ACETATE:Chemotherapy:0.2), (ETORICOXIB:Chemotherapy:0.89), (ETRETINATE:Chemotherapy: 0.37), (FENOFIBRIC ACID:Chemotherapy:0.21), (FENOTEROL HYDROBROMIDE\IPRATROPIUM BROMIDE:Chemotherapy:0.01), (FERROUS SULFATE\GLYCINE:Chemotherapy:0.15), (FINGOLIMOD HYDROCHLORIDE:Chemotherapy:0.83), (FLUINDIONE:Chemotherapy:0.7), (FLUTAMIDE: Chemotherapy:0.05), (FORMOTEROL:Chemotherapy:0.89), (FORMOTEROL FUMARATE\MOMETASONE FUROATE:Chemotherapy:0.05), (FUSIDIC ACID:Chemotherapy:1.03), (GLYCOPYRROLATE:Chemotherapy:0.39), (GLYCOSAMINOGL YCANS:Chemotherapy:0.15), (HERBALS\TURMERIC:Chemotherapy:0.63), (HYDROCHLOR OTHIAZIDE\QUINAPRIL HYDROCHLORIDE:Chemotherapy:0.04), (HYDROCORTISONE ACETATE:Chemotherapy:0.05), (ILOPROST:Chemotherapy:0.05), (IMIGLUCERASE:Chemotherapy: 0.08), (IMIPRAMINE HYDROCHLORIDE:Chemotherapy:0.54), (INTERFERON ALFA-2A OR INTERFERON ALFA-2B:Chemotherapy:0.48), (INTERFERON GAMMA-1B:Chemotherapy:0.33), (IODINE:Chemotherapy:0.07), (LERCANIDIPINE HYDROCHLORIDE:Chemotherapy:1.47), (LIOTHYRONINE SODIUM:Chemotherapy:0.34), (LUTEIN:Chemotherapy:0.04), (MAGNESIUM ASPARTATE\POTASSIUM ASPARTATE:Chemotherapy:0.08), (MECLIZINE\MECLIZINE HYDROCHLORIDE:Chemotherapy:1.06), (MEFENAMIC ACID:Chemotherapy:0.17), (METAXALONE:Chemotherapy:1.1), (METHENOLONE ENANTHATE:Chemotherapy:0.75), (METOLAZONE:Chemotherapy:0.11), (MUPIROCIN\MU PIROCIN CALCIUM:Chemotherapy:0.3), (NALOXONE:Chemotherapy:0.25), (NALOXONE HYDROCHLORIDE\TILIDINE HYDROCHLORIDE:Chemotherapy:0.55), (NICARDIPINE:Chemotherapy:0.59), (NITROFURA NTOIN\NITROFURANTOIN MONOHYDRATE:Chemotherapy:0.55), (NOREPINEPHRINE:Chemotherapy:0.38), (OLMESA RTAN:Chemotherapy:0.68), (OLODATEROL HYDROCHLORIDE\TIOTROPIUM BROMIDE MONOHYDRATE:Chemotherapy:0.78), (OMEGA-3-ACID ETHYL ESTERS:Chemotherapy:1.35), (OSELTAMIVIR:Chemotherapy:0.52), (OSELTAMIVIR PHOSPHATE:Chemotherapy:0.53), (PANTOPRAZOLE MAGNESIUM:Chemotherapy:1.46), (PEGINTERFERON ALFA-2A:Chemotherapy:0.54), (PIRFENIDONE:Chemotherapy:0.06), (PIROXICAM:Chemotherapy:0.57), (PRAZOSIN HYDROCHLORIDE:Chemotherapy:0.08), (PYRIDOSTIGMINE BROMIDE:Chemotherapy:0.05), (RETINOL:Chemotherapy:0.54), (RIVASTIGMINE:Chemotherapy: 0.23), (RIVASTIGMINE TARTRATE:Chemotherapy:0.21), (RUCAPARIB CAMSYLATE:Chemotherapy:0.31), (SAMARIUM SM-153 LEXIDRONAM PENTASODIUM:Chemotherapy:0.36), (SODIUM OXYBATE:Chemotherapy:0.59), (SOMATOSTATIN:Chemotherapy:0.05), (SUMATRIPTAN:Chemotherapy: 0.69), (SUMATRIPTAN\SUMATRIPTAN SUCCINATE:Chemotherapy:0.96), (TEDUGLUTIDE\WATER:Chemotherapy:0.07), (TELOTRI STAT ETHYL:Chemotherapy:0.48), (TERBUTALINE:Chemotherapy:0.26), (TERIFLUNOMIDE:Chemotherapy: 0.16), (TIMOLOL MALEATE:Chemotherapy:0.01), (TOBRAMYCIN:Chemotherapy:0.37), (TRANDOLAPRIL:Chemotherapy: 0.13), (TRIPTORELIN PAMOATE:Chemotherapy:0.09), (TROSPIUM CHLORIDE:Chemotherapy:0.46), (UMECLIDINIUM BROMIDE:Chemotherapy:0.1), (UMECLIDINIUM BROMIDE\VILANTEROL TRIFENATATE:Chemotherapy:0.69), (URAPIDIL:Chemotherapy:0), (VANDETANIB:Chemotherapy: 0.21), (VILAZODONE HYDROCHLORIDE:Chemotherapy:0.06), (VISMODEGIB:Chemotherapy:0.02), (ZINC:Chemotherapy: 0.57), (.ALPHA.1-PROTEINASE INHIBITOR HUMAN:Chest discomfort:0.6), (ALBIGLUTIDE:Chest discomfort:0.03), (BISACODYL OR DOCUSATE SODIUM:Chest discomfort:0.23), (BORTEZOMIB:Chest discomfort:0.05), (BUDESONIDE\FORMOTEROL FUMARATE:Chest discomfort:0.12), (CALCIUM ACETATE:Chest discomfort:0.05), (CALCIUM\VITAMIN D:Chest discomfort:0.5), (CANDESARTAN CILEXETIL:Chest discomfort:0.45), (CANDESARTAN CILEXETIL\HYDROCHLOROTHIAZIDE:Chest discomfort:0.04), (CARVEDILOL PHOSPHATE:Chest discomfort:0.23), (CLOTRIMAZOLE:Chest discomfort:0.26), (CORTISONE\HYDROCORTISONE:Chest discomfort:0.37), (DACLIZUMAB:Chest discomfort:0.05), (DALFAMPRIDINE:Chest discomfort:0.55), (DASATINIB:Chest discomfort:0.27), (DEVICE:Chest discomfort:0.17), (DICLOFENAC:Chest discomfort:0.42), (DIVALPROEX SODIUM:Chest discomfort:0.15), (ELETRIPTAN HYDROBROMIDE:Chest discomfort:0.42), (ENALAPRIL MALEATE:Chest discomfort:0.09), (ESZOPICLONE:Chest discomfort:0.56), (EVEROLIMUS:Chest discomfort:0.05), (FILGRASTIM:Chest discomfort:0.01), (FLECAINIDE:Chest discomfort:0.16), (FLUTICASONE:Chest discomfort:0.33), (FLUTICASONE FUROATE\SALMETEROL XINAFOATE:Chest discomfort:0.07), (GLYCOPYRRONIUM\INDACATEROL:Chest discomfort:0), (HYDROCHLOROTHIAZIDE\LISINOPRIL:Chest discomfort:0.48), (HYDROMORPHONE:Chest discomfort:0.17), (HYDROXYCHLOROQUINE DIPHOSPHATE:Chest discomfort:0.08), (HYOSCYAMINE:Chest discomfort:0.26), (ILOPROST:Chest discomfort:0.47), (INDOMETHACIN:Chest discomfort:0.3), (LATANOPROST:Chest discomfort:0.06), (MEPOLIZUMAB:Chest discomfort:0.56), (METHADONE HYDROCHLORIDE:Chest discomfort:0.31), (METRONIDAZOLE:Chest discomfort:1.21), (MOXIFLOXACIN HYDROCHLORIDE:Chest discomfort:0.35), (OLANZAPINE:Chest discomfort:0), (OLODATEROL HYDROCHLORIDE\TIOTROPIUM BROMIDE MONOHYDRATE:Chest discomfort:0.02), (OMEGA-3-ACID ETHYL ESTERS:Chest discomfort:0.2), (PANTOPRAZOLE:Chest discomfort:0.96), (PAROXETINE:Chest discomfort:0.24), (PAROXETINE\PAROXETINE HYDROCHLORIDE:Chest discomfort:0.23), (PRAMIPEXOLE:Chest discomfort:0.01), (PRIMIDONE:Chest discomfort:0), (PROBIOTICS NOS:Chest discomfort:0.44), (RABEPRAZOLE:Chest discomfort:0.03), (SECUKINUMAB:Chest discomfort:0.03), (SODIUM OXYBATE:Chest discomfort:0.43), (SOTALOL:Chest discomfort:0.18), (TERIFLUNOMIDE:Chest discomfort:0.61), (ABOBOTULINUMTOXINA:Chest pain:0.13), (ACETAMINOPHEN\CAFFEINE:Chest pain:0.33), (AGALSIDASE BETA:Chest pain:1.4), (ALDESLEUKIN:Chest pain:0.23), (AMILORIDE HYDROCHLORIDE\HYDROCHLOROTHIAZIDE:Chest pain:1.02), (AMINO ACIDS\DEXTROSE\ELECTROLYTES NOS:Chest pain:0.74), (AMLODIPINE BESYLATE\TELMISARTAN:Chest pain:0.06), (AMMONIUM CHLORIDE\DEXTROMETHORPHAN\SODIUM CITRATE:Chest pain:0.37), (AMOXICILLIN\CLARITHROMYCIN\ESOMEPRAZOLE:Chest pain:0.36), (ANAGRELIDE HYDROCHLORIDE:Chest pain:0.46), (ANTIHISTAMINES NOS:Chest pain:0.73), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Chest pain:0.24), (ASPIRIN\CLOPIDOGREL:Chest pain:0.34), (ATOVAQUONE:Chest pain:0.57), (BELATACEPT:Chest pain:0.29), (BENZTROPINE MESYLATE:Chest pain:0.23), (BETAMETHASONE\CALCIPOTRIENE:Chest pain:0.55), (BETHANECHOL CHLORIDE:Chest pain:0.18), (BIFIDOBACTERIUM LONGUM SUBSP. INFANTIS:Chest pain:1.27), (BILASTINE:Chest pain:0.21), (BLINATUMOMAB:Chest pain:0.5), (BUPRENORPHINE HYDROCHLORIDE:Chest pain:1.64), (BUTYLSCOPOLAMINE BROMIDE:Chest pain:0.74), (CABAZITAXEL:Chest pain:0.08), (CABOZANTINIB S-MALATE:Chest pain:0.43), (CARBIMAZOLE:Chest pain:0.71), (CASPOFUNGIN:Chest pain:0.44), (CEFTAZIDIME:Chest pain:0.85), (CELIPROLOL:Chest pain:0), (CETUXIMAB:Chest pain:0.37), (CILASTATIN SODIUM\IMIPENEM:Chest pain:1.49), (CIMETIDINE:Chest pain:2.51), (CLIKSTAR:Chest pain:0.26), (CLOMIPRAMINE HYDROCHLORIDE:Chest pain:0.62), (COLISTIMETHATE SODIUM:Chest pain:0.4), (COLISTIN:Chest pain:0.21), (DABRAFENIB MESYLATE:Chest pain:0.48), (DANTROLENE SODIUM:Chest pain:0.27), (DAPAGLIFLOZIN PROPANEDIOL\METFORMIN HYDROCHLORIDE:Chest pain:0.13), (DAPSONE:Chest pain:0.74), (DEFLAZACORT:Chest pain:1.65), (DEGARELIX:Chest pain:0.2), (DEXAMETHASONE ACETATE:Chest pain:1.05), (DEXTROAMPHETAMINE SULFATE:Chest pain:0.81), (DIGITOXIN:Chest pain:1.28), (DOLASETRON MESYLATE:Chest pain:0.65), (DOXYLAMINE SUCCINATE:Chest pain:0.66), (ECONAZOLE:Chest pain:1.52), (ELTROMBOPAG OLAMINE:Chest pain:0.54), (EMPAGLIFLOZIN\LINAGLIPTIN:Chest pain:1.29), (EPINASTINE HYDROCHLORIDE:Chest pain:0.15), (EPIRUBICIN:Chest pain:0.44), (ERIBULIN:Chest pain:0.13), (ESTROGENS:Chest pain:0.49), (ESTRONE\SUS SCROFA OVARY:Chest pain:0.17), (ETHACRYNIC ACID:Chest pain:0.2), (ETHINYL ESTRADIOL\NORGESTREL:Chest pain:0.17), (ETRETINATE:Chest pain:0.11), (FENOTEROL HYDROBROMIDE:Chest pain:0.9), (FENOTEROL HYDROBROMIDE\IPRATROPIUM BROMIDE:Chest pain:1.02), (FLUOROMETHOLONE:Chest pain:0.25), (FLURAZEPAM\FLURAZEPAM HYDROCHLORIDE:Chest pain:0.11), (FORMOTEROL FUMARATE\MOMETASONE FUROATE:Chest pain:1.44), (FOSFOMYCIN:Chest pain:0.13), (FUSIDIC ACID:Chest pain:1.17), (GEMCITABINE HYDROCHLORIDE:Chest pain:0.64), (GIMERACIL\OTERACIL\TEGAFUR:Chest pain:0.62), (GLIMEPIRIDE\ROSIGLITAZONE MALEATE:Chest pain:0.36), (GLUTAMINE:Chest pain:0.07), (GLYCOPYRROLATE\INDACATEROL MALEATE:Chest pain:0.52), (GUANFACINE:Chest pain:0.65), (GUANFACINE HYDROCHLORIDE:Chest pain:0.17), (HYDROCHLOROTHIAZIDE\RAMIPRIL:Chest pain:0.14), (IDARUCIZUMAB:Chest pain:0.38), (ILOPROST:Chest pain:1.65), (IMIGLUCERASE:Chest pain:0.08), (INDAPAMIDE\PERINDOPRIL:Chest pain:2.17), (INFLIXIMAB-DYYB:Chest pain:0.55), (INOTUZUMAB OZOGAMICIN:Chest pain:0.11), (INTERFERON:Chest pain:0.73), (INTERFERON ALFA-2A:Chest pain:1.23), (INTERFERON ALFA-2B:Chest pain:1.02), (INTERFERON GAMMA-1B:Chest pain:0.35), (LANREOTIDE:Chest pain:0.27), (LAPATINIB DITOSYLATE:Chest pain:0.72), (LEUCOVORIN CALCIUM:Chest pain:0.68), (LEVOLEUCOVORIN CALCIUM:Chest pain:0.11), (LEVOMEFOLATE CALCIUM:Chest pain:0.39), (LEVOTHYROXINE SODIUM\POTASSIUM IODIDE:Chest pain:0.17), (LINEZOLID:Chest pain:0.55), (LUMACAFTOR:Chest pain:0.09), (LUTETIUM LU-177:Chest pain:0.1), (MAGNESIUM ASPARTATE\POTASSIUM ASPARTATE:Chest pain:0.11), (MEDROXYPROGESTERONE:Chest pain:0.08), (MELPHALAN:Chest pain:0.11), (MEPOLIZUMAB:Chest pain:1.34), (MESNA:Chest pain:0.27), (METFORMIN HYDROCHLORIDE\ROSIGLITAZONE MALEATE:Chest pain:0.67), (METFORMIN HYDROCHLORIDE\SAXAGLIPTIN:Chest pain:0.15), (METHENAMINE HIPPURATE:Chest pain:1.85), (METHENOLONE ENANTHATE:Chest pain:0.06), (MINOCYCLINE HYDROCHLORIDE:Chest pain:1.24), (NARATRIPTAN HYDROCHLORIDE:Chest pain:0.23), (NICARDIPINE:Chest pain:1.14), (NIRAPARIB:Chest pain:1.19), (NOREPINEPHRINE BITARTRATE:Chest pain:0.77), (OLAPARIB:Chest pain:0.63), (OSPEMIFENE:Chest pain:0.38), (OXAPROZIN:Chest pain:0.28), (OXYBUTYNIN\OXYBUTYNIN CHLORIDE:Chest pain:0.41), (PANITUMUMAB:Chest pain:0.11), (PANTOPRAZOLE MAGNESIUM:Chest pain:2.77), (PARICALCITOL:Chest pain:0.97), (PASIREOTIDE:Chest pain:0.21), (PEGINTERFERON ALFA-2B:Chest pain:0.1), (PEGINTERFERON BETA-1A:Chest pain:1.47), (PENTOSTATIN:Chest pain:0.21), (PHENPROCOUMON:Chest pain:1.01), (PILSICAINIDE HYDROCHLORIDE:Chest pain:0.14), (POLYETHYLENE GLYCOL 3350\POTASSIUM CHLORIDE\SODIUM BICARBONATE\SODIUM CHLORIDE\SODIUM SULFATE ANHYDROUS:Chest pain:0.49), (PRAMLINTIDE ACETATE:Chest pain:0), (PREDNISOLONE SODIUM PHOSPHATE:Chest pain:0.48), (PREDNISOLONE\PREDNISOLONE ACETATE\PREDNISOLONE SODIUM SUCCINATE:Chest pain:1.38), (PRISTINAMYCIN:Chest pain:0.17), (PROGESTERONE:Chest pain:0.87), (PROTRIPTYLINE HYDROCHLORIDE:Chest pain:0.36), (QUINACRINE:Chest pain:0.03), (RADIATION THERAPY:Chest pain:1.25), (RAMUCIRUMAB:Chest pain:0.6), (REPAGLINIDE:Chest pain:0.72), (RETINOL:Chest pain:1.14), (RIFAMPIN:Chest pain:0.81), (RILMENIDINE PHOSPHATE:Chest pain:0.46), (RIZATRIPTAN:Chest pain:0.48), (SELEXIPAG:Chest pain:0.47), (SEVELAMER:Chest pain:0.02), (SEVELAMER HYDROCHLORIDE:Chest pain:1.02), (SEVOFLURANE:Chest pain:0.36), (SODIUM CITRATE:Chest pain:0.2), (SOFOSBUVIR\VELPATASVIR:Chest pain:0.34), (TAFAMIDIS MEGLUMINE:Chest pain:0.05), (TAFLUPROST:Chest pain:0.08), (TEDUGLUTIDE\WATER:Chest pain:0.58), (TEMSIROLIMUS:Chest pain:0.23), (TESTOSTERONE UNDECANOATE:Chest pain:0.98), (TRAMETINIB DIMETHYL SULFOXIDE:Chest pain:0.41), (TRANDOLAPRIL:Chest pain:0.7), (TRIMIPRAMINE MALEATE:Chest pain:0.26), (VASOPRESSIN:Chest pain:0.13), (VINORELBINE TARTRATE:Chest pain:0.8), (WARFARIN POTASSIUM:Chest pain:0.04), (ALENDRONIC ACID:Chills:0.02), (AMIODARONE:Chills:0.32), (AMITRIPTYLINE:Chills:0.04), (ANASTRO ZOLE:Chills:0.35), (BUMETANIDE:Chills:0.48), (CANDESARTAN CILEXETIL:Chills:0.09), (CELECOXIB:Chills:0.3), (CEPHALEXIN:Chills:0.67), (CERTOLIZU MAB PEGOL:Chills:0.12), (CLOZAPINE:Chills:0.22), (DALFAMPRIDINE:Chills:0.22), (DASATINI B:Chills:0.37), (DILTIAZEM:Chills:0.32), (EPIRUBICIN:Chills:0.03), (EVEROLIMUS:Chills:0.22), (FERROUS SULFATE:Chills:0.2), (IRON:Chills:0.1), (ISOSORBIDE MONONITRATE:Chills:0.3), (MELATONIN:Chills:0.19), (NEBIVOLOL:Chills:0.14), (NITROF URANTOIN:Chills:0.08), (OLANZAPINE:Chills:0.27), (OXCARBAZEPINE:Chills:0.33), (SAC UBITRIL\VALSARTAN:Chills:0.8), (TERIFLUNOMIDE:Chills:0.1), (THALIDOMIDE:Chills: 0.93), (AMIODARONE:Cholangiocarcinoma:0.02), (BISOPROLOL FUMARATE:Cholangiocarcinoma:0.03), (CALCIUM CARBONATE\CHOLECALCIFEROL:Cholangiocarcinoma:0.34), (CANDESARTAN:Cholangiocarcinoma: 0.02), (CLOPIDOGREL BISULFATE:Cholangiocarcinoma:0.41), (DIGOXIN:Cholangiocarcinoma:0.57), (DILTIAZEM: Cholangiocarcinoma:0.18), (ENALAPRIL:Cholangiocarcinoma:0.55), (EXEMESTANE:Cholangiocarcinoma: 0.47), (FERROUS SULFATE\FERROUS SULFATE, DRIED:Cholangiocarcinoma:0.07), (FLUTICASONE PROPIONATE:Cholangiocarcinoma:0.31), (LENALIDOMIDE:Cholangiocarcinoma:0.11), (LEV OLEUCOVORIN CALCIUM:Cholangiocarcinoma:0.43), (PEMETREXED:Cholangiocarcinoma:0.01), (POTASSI UM:Cholangiocarcinoma:0.01), (PRAVASTATIN:Cholangiocarcinoma:0.07), (RAMUCIRUMA B:Cholangiocarcinoma:0.02), (VALSARTAN:Cholangiocarcinoma:0.1), (AMIODARONE:Cholelithiasis: 0.48), (AMIODARONE HYDROCHLORIDE:Cholelithiasis:0.06), (ANASTROZOLE:Cholelithiasis:0.33), (BUDESONI DE:Cholelithiasis:0.75), (BUMETANIDE:Cholelithiasis:0.16), (CALCITRIOL:Cholelithiasis:0.05), (CALCIUM CARBONATE\CHOLECALCIFEROL:Cholelithiasis:0.39), (CEPHALEXIN:Cholelithiasis:0), (CLONAZEPAM:Cholelithiasis:0.4), (COLCHICINE:Cholelithiasis:0.06), (DASATINIB:Cholelithiasis: 0.39), (DEXLANSOPRAZOLE:Cholelithiasis:0.24), (DIGOXIN:Cholelithiasis:0.45), (DIL TIAZEM:Cholelithiasis:0.02), (ENOXAPARIN:Cholelithiasis:0.27), (EVOLOCUMAB:Cholelithiasis: 0.58), (FERROUS SULFATE\FERROUS SULFATE, DRIED:Cholelithiasis:0.67), (HYDROCHLOROTHIAZIDE\LISINOPRIL:Cholelithiasis:0.32), (I BRUTINIB:Cholelithiasis:1.05), (IMATINIB MESYLATE:Cholelithiasis:0.41), (LOVASTATIN:Cholelithiasis:0.47), (MELOXICAM:Cholelithiasis: 0.21), (METHYLPREDNISOLONE SODIUM SUCCINATE:Cholelithiasis:0.41), (METRONIDAZOLE\METRONIDAZOLE HYDROCHLORIDE:Cholelithiasis:0.09), (NILOTINIB:Cholelithiasis:0.53), (OXCARBAZEPIN E:Cholelithiasis:0.2), (PAZOPANIB HYDROCHLORIDE:Cholelithiasis:0.03), (PROMETHAZINE\PROMETHAZINE HYDROCHLORIDE:Cholelithiasis:0.08), (RANOLAZINE:Cholelithiasis:0.21), (SACUBITRIL\VALSARTAN:Cholelithiasis:0.18), (SECUKINUMAB:Cholelithiasis:0.08), (TAMSULOSIN:Cholelithiasis: 0.55), (PYRAZINAMIDE:Chorioretinitis:0.58), (OMEPRAZOLE MAGNESIUM:Choroidal neovascularisation:0.08), (POLYMYXIN B SULFATE\TRIMETHOPRIM SULFATE:Choroidal neovascularisation:0.11), (ACETAMINOPHEN\BUTALBITAL\CAFFEINE:Chronic fatigue syndrome:0.38), (CERTOLIZUMAB PEGOL:Chronic fatigue syndrome:0.29), (CHOLESTYRAMINE:Chronic fatigue syndrome:0.34), (CIPROFLOXACIN HYDROCHLORIDE:Chronic fatigue syndrome:0.41), (DACLIZUMAB:Chronic fatigue syndrome:0.29), (DALFAMPRIDINE:Chronic fatigue syndrome:0.39), (DICLOFENAC SODIUM:Chronic fatigue syndrome:0.26), (DIMENHYDRINATE:Chronic fatigue syndrome:0.04), (DOCUSATE SODIUM:Chronic fatigue syndrome:0.72), (EVOLOCUMAB:Chronic fatigue syndrome:0.13), (INDOMETHACIN:Chronic fatigue syndrome:0.02), (PANTOPRAZOLE:Chronic fatigue syndrome:0.32), (PAROXETINE:Chronic fatigue syndrome:0.08), (PRAVASTATIN SODIUM:Chronic fatigue syndrome:0.17), (PROMETHAZINE HYDROCHLORIDE:Chronic fatigue syndrome:0.01), (RISEDRONATE SODIUM:Chronic fatigue syndrome:0.14), (RIZATRIPTAN BENZOATE:Chronic fatigue syndrome:0.51), (TERIFLUNOMIDE:Chronic fatigue syndrome:0.58), (VARENICLINE TARTRATE:Chronic fatigue syndrome:0.91), (VERAPAMIL HYDROCHLORIDE:Chronic fatigue syndrome:0.02), (VILAZODONE HYDROCHLORIDE:Chronic fatigue syndrome:0.22), (.ALPHA.-TOCOPHEROL\RETINOL:Chronic gastritis:0.77), (ACETAMINOPHEN\APRONALIDE\CAFFEINE\PROPYPHENAZONE:Chronic gastritis:0.74), (ACETAMINOPHEN\BROMISOVAL\DIHYDROCODEINE PHOSPHATE\DIPHENHYDRAMINE SALICYLATE\DYPHYLLINE\METHYLEPHEDRINE HYDROCHLORIDE, (+/−)-:Chronic gastritis:0.29), (ACETAMINOPHEN\PSEUDOEPHEDRINE HYDROCHLORIDE:Chronic gastritis:0.19), (ACTIVATED CHARCOAL:Chronic gastritis:0.81), (ADENINE:Chronic gastritis:1.55), (ALCOHOL:Chronic gastritis:0.1), (ALENDRONIC ACID:Chronic gastritis:0.83), (ALUMINUM SILICATE:Chronic gastritis:0.84), (AMISULPRIDE:Chronic gastritis:0.73), (AMPICILLIN SODIUM\SULBACTAM SODIUM:Chronic gastritis:0.45), (APRINDINE HYDROCHLORIDE:Chronic gastritis:0.62), (AROTINOLOL:Chronic gastritis:0.02), (ASPIRIN\DIHYDROXYALUMINUM AMINOACETATE\MAGNESIUM CARBONATE:Chronic gastritis:0.12), (AXITINIB:Chronic gastritis:0.16), (BENAZEPRIL HYDROCHLORIDE:Chronic gastritis:0.44), (BETAMETHASONE BUTYRATE PROPIONATE:Chronic gastritis:0.94), (BETAMETHASONE SODIUM PHOSPHATE\NEOMYCIN SULFATE:Chronic gastritis:0.64), (BICARBONATE ION\POLYETHYLENE GLYCOLS\POTASSIUM CATION\SODIUM CHLORIDE\SODIUM SULFATE ANHYDROUS:Chronic gastritis:0.27), (BISACODYL:Chronic gastritis:0.49), (BISACODYL OR DOCUSATE SODIUM:Chronic gastritis:0.55), (BROMHEXINE HYDROCHLORIDE:Chronic gastritis:0.46), (BUDESONIDE\FORMOTEROL FUMARATE:Chronic gastritis:0.37), (BUFORMIN:Chronic gastritis:0.27), (BUMETANIDE:Chronic gastritis:1.05), (BUPRENORPHINE HYDROCHLORIDE:Chronic gastritis:0.63), (BUPROPION:Chronic gastritis:0.3), (BUSPIRONE HYDROCHLORIDE:Chronic gastritis:0.11), (CALCIUM ACETATE:Chronic gastritis:0.26), (CALCIUM CHLORIDE ANHYDROUS\POTASSIUM CHLORIDE\SODIUM CHLORIDE\SODIUM LACTATE:Chronic gastritis:0.71), (CALCIUM CHLORIDE\DEXTROSE\POTASSIUM CHLORIDE\SODIUM ACETATE\SODIUM CHLORIDE:Chronic gastritis:0.72), (CALCIUM CITRATE:Chronic gastritis:0.01), (CALCIUM\VITAMIN D:Chronic gastritis:0.46), (CAMPHOR\CAPSICUM\METHYL SALICYLATE:Chronic gastritis:0.1), (CAMPHOR\MENTHOL\METHYL SALICYLATE:Chronic gastritis:0.75), (CANAGLIFLOZIN\METFORMIN HYDROCHLORIDE:Chronic gastritis:0.26), (CARBOHYDRATES\POTASSIUM CHLORIDE\SODIUM CHLORIDE\SODIUM LACTATE:Chronic gastritis:1.03), (CARFILZOMIB:Chronic gastritis:0.36), (CARVEDILOL PHOSPHATE:Chronic gastritis:0.06), (CEFTERAM PIVOXIL:Chronic gastritis:0.16), (CELLULASE\DIASTASE\LIPASE:Chronic gastritis:0.95), (CEPHALEXIN:Chronic gastritis:1.43), (CERITINIB:Chronic gastritis:0.19), (CHIMAPHILA UMBELLATA\MANGANESE CHLORIDE\POPULUS TREMULOIDES\SODIUM TAUROCHOLATE:Chronic gastritis:0.69), (CHLORPROMAZINE\PROMETHAZINE:Chronic gastritis:0.56), (CILASTATIN SODIUM\IMIPENEM:Chronic gastritis:0.56), (CLENBUTEROL HYDROCHLORIDE:Chronic gastritis:0.5), (CLINDAMYCIN\CLINDAMYCIN PHOSPHATE:Chronic gastritis:0.71), (CLOPERASTINE:Chronic gastritis:0.8), (CLOTRIMAZOLE:Chronic gastritis:0.37), (COLESTILAN CHLORIDE:Chronic gastntis:0.03), (CORTISONE\HYDROCORTISONE:Chronic gastntis:1.07), (CYANAMIDE:Chronic gastritis:0.15), (CYANOCOBALAMIN\LIDOCAINE HYDROCHLORIDE\PYRIDOXINE HYDROCHLORIDE\THIAMINE HYDROCHLORIDE:Chronic gastritis:0.39), (CYCLOBENZAPRINE HYDROCHLORIDE:Chronic gastritis:0.06), (DABRAFENIB:Chronic gastritis:0.06), (DAPAGLIFLOZIN PROPANEDIOL:Chronic gastritis:0.1), (DEFLAZACORT:Chronic gastritis:0.56), (DEVICE:Chronic gastritis:0.21), (DIFLUCORTOLONE VALERATE:Chronic gastritis:0.45), (DIFLUCORTOLONE VALERATE\LIDOCAINE:Chronic gastritis:0.26), (DIGITOXIN:Chronic gastritis:0.36), (DIMENHYDRINATE:Chronic gastritis:0.11), (DIPHENIDOL HYDROCHLORIDE:Chronic gastritis:1.53), (DIQUAFOSOL TETRASODIUM:Chronic gastritis:0.52), (DISOPYRAMIDE:Chronic gastritis:0.36), (DOCUSATE:Chronic gastritis:0.68), (DOCUSATE SODIUM:Chronic gastritis:1.11), (DOXYCYCLINE:Chronic gastritis:0.29), (DRONABINOL:Chronic gastritis:0.14), (DULOXETINE:Chronic gastritis:0.22), (ELETRIPTAN HYDROBROMIDE:Chronic gastritis:0), (ELOTUZUMAB:Chronic gastritis:0.17), (EPRAZINONE:Chronic gastritis:0.23), (ERYTHROMYCIN LACTOBIONATE:Chronic gastritis:0.06), (ETORICOXIB:Chronic gastritis:0.97), (EVOLOCUMAB:Chronic gastritis:0.56), (FAROPENEM SODIUM:Chronic gastritis:1.02), (FENOTEROL HYDROBROMIDE\IPRATROPIUM BROMIDE:Chronic gastritis:0.24), (FERRIC HYDROXIDE:Chronic gastritis:0.69), (FERRIC SODIUM CITRATE:Chronic gastritis:0.3), (FERROUS CITRATE\SODIUM:Chronic gastritis:0.07), (FISH OIL:Chronic gastritis:0.51), (FLECAINIDE:Chronic gastritis:0.83), (FLUOXETINE\FLUOXETINE HYDROCHLORIDE:Chronic gastritis:0.19), (FLUTICASONE:Chronic gastritis:0.34), (FOSFOMYCIN CALCIUM:Chronic gastritis:0.55), (FULVESTRANT:Chronic gastritis:0.82), (FUSIDIC ACID:Chronic gastritis:0.31), (GABEXATE MESYLATE:Chronic gastritis:0.08), (GUAIAZULENE:Chronic gastritis:0.16), (HEPARIN CALCIUM:Chronic gastritis:0.23), (HERBS\ROOTS:Chronic gastritis:0), (HYDROCORTISONE PROBUTATE:Chronic gastritis:0.02), (HYDROMORPHONE:Chronic gastritis:0.39), (HYDROXYZINE PAMOATE:Chronic gastritis:0.29), (IDARUCIZUMAB:Chronic gastritis:0.01), (IFENPRODIL TARTRATE:Chronic gastritis:0.64), (ILOPROST:Chronic gastritis:0.09), (IMIPRAMINE HYDROCHLORIDE:Chronic gastritis:0.57), (IMMUNE GLOBULIN NOS:Chronic gastntis:0.21), (INDAPAMIDE\PERINDOPRIL:Chronic gastritis:0.51), (INFLUENZA VIRUS VACCINE:Chronic gastritis:0.31), (INSULIN ASPART\INSULIN DEGLUDEC:Chronic gastritis:0.36), (IPRATROPIUM:Chronic gastritis:0.59), (IRINOTECAN:Chronic gastritis:0.73), (IRINOTECAN HYDROCHLORIDE:Chronic gastritis:0.93), (IRON:Chronic gastritis:0.82), (ISOSORBIDE:Chronic gastritis:0.66), (IXAZOMIB:Chronic gastritis:0.14), (LABETALOL\LABETALOL HYDROCHLORIDE:Chronic gastritis:0.39), (LEVALBUTEROL HYDROCHLORIDE:Chronic gastritis:0.05), (LEVOCARNITINE HYDROCHLORIDE:Chronic gastritis:1.59), (LEVOLEUCOVORIN CALCIUM:Chronic gastritis:0.86), (LICORICE ROOT EXTRACT\RHUBARB:Chronic gastritis:0.55), (LIDOCAINE\PRILOCAINE:Chronic gastritis:0.05), (LISDEXAMFETAMINE DIMESYLATE:Chronic gastritis:0.55), (MEPENZOLATE BROMIDE:Chronic gastritis:0.4), (MEPOLIZUMAB:Chronic gastritis:0.14), (MIGLUSTAT:Chronic gastritis:0.1), (MIRIPLATIN:Chronic gastritis:0.35), (NAFAMOSTAT MESYLATE:Chronic gastritis:0.25), (NIACIN:Chronic gastritis:0.08), (NILVADIPINE:Chronic gastritis:0.17), (OK-432:Chronic gastritis:0.02), (PANTOPRAZOLE MAGNESIUM:Chronic gastritis:0.62), (PAROXETINE\PAROXETINE HYDROCHLORIDE:Chronic gastritis:0.24), (PEGVISOMANT:Chronic gastritis:0.14), (PEMETREXED:Chronic gastntis:0.67), (PENTAZOCINE:Chronic gastritis:0.2), (PEROSPIRONE HYDROCHLORIDE:Chronic gastritis:0.34), (PHENYTOIN:Chronic gastritis:0.74), (POLYETHYLENE GLYCOL 3350:Chronic gastritis:0.25), (POLYETHYLENE GLYCOLS:Chronic gastritis:1.19), (PONATINIB:Chronic gastritis:0.18), (POTASSIUM CHLORIDE\SODIUM CHLORIDE\SODIUM LACTATE:Chronic gastritis:0.7), (POTASSIUM CITRATE\TRISODIUM CITRATE DIHYDRATE:Chronic gastritis:1.45), (POTASSIUM IODIDE:Chronic gastritis:0.63), (PREDNISOLONE VALERATE ACETATE:Chronic gastntis:0.3), (PRIMIDONE:Chronic gastritis:0.59), (PROMETHAZINE\PROMETHAZINE HYDROCHLORIDE:Chronic gastritis:0.28), (PROPRANOLOL\PROPRANOLOL HYDROCHLORIDE:Chronic gastritis:0.09), (PYRAZINAMIDE:Chronic gastritis:0.73), (PYRIDOXAL PHOSPHATE:Chronic gastritis:0.15), (PYRIDOXINE HYDROCHLORIDE:Chronic gastritis:0.02), (RAMUCIRUMAB:Chronic gastritis:1.01), (RANOLAZINE:Chronic gastritis:1.01), (REPAGLINIDE:Chronic gastritis:0.34), (RILPIVIRINE HYDROCHLORIDE:Chronic gastritis:0.11), (ROFLUMILAST:Chronic gastritis:0.52), (ROPINIROLE HYDROCHLORIDE:Chronic gastritis:0.16), (ROSUVASTATIN:Chronic gastritis:0.08), (SARPOGRELATE HYDROCHLORIDE:Chronic gastritis:1.2), (SENNOSIDE A:Chronic gastritis:0.07), (SENNOSIDE B:Chronic gastritis:0.06), (SERRAPEPTASE:Chronic gastritis:0.12), (SITAFLOXACIN:Chronic gastritis:0.47), (SIVELESTAT SODIUM:Chronic gastritis:0.39), (SOTALOL:Chronic gastritis:0.77), (SUPLATAST TOSILATE:Chronic gastritis:1.39), (TANDOSPIRONE:Chronic gastritis:0.24), (TEMAZEPAM:Chronic gastritis:0.6), (TEMOCAPRIL HYDROCHLORIDE:Chronic gastritis:1.22), (TERBUTALINE SULFATE:Chronic gastritis:0.29), (THALIDOMIDE:Chronic gastritis:1.53), (THROMBOMODULIN ALFA:Chronic gastritis:0.18), (TIARAMIDE:Chronic gastritis:0.14), (TICLOPIDINE:Chronic gastritis:0.14), (TIMOLOL:Chronic gastritis:0.19), (TINZAPARIN SODIUM:Chronic gastritis:0.24), (TIZANIDINE HYDROCHLORIDE:Chronic gastritis:0.3), (TOPIROXOSTAT:Chronic gastritis:0.16), (TRAMETINIB:Chronic gastritis:0.07), (UMECLIDINIUM BROMIDE\VILANTEROL TRIFENATATE:Chronic gastritis:0.04), (UROKINASE:Chronic gastritis:0.29), (VITAMIN B:Chronic gastritis:0.14), (BUMETANIDE:Chronic graft versus host disease:0.01), (CLINDAMYCIN\CLINDAMYCIN PHOSPHATE:Chronic graft versus host disease:0), (DILTIAZEM:Chronic graft versus host disease:0.04), (DIPHENHYDRAMINE:Chronic graft versus host disease:0.05), (RAMIPRIL:Chronic graft versus host disease:0.05), (ALBUTEROL:Chronic hepatitis B:0.13), (AMIODARONE HYDROCHLORIDE:Chronic hepatitis B:0.26), (BISOPROLOL FUMARATE:Chronic hepatitis B:0.57), (CALCIUM CARBONATE\CHOLECALCIFEROL:Chronic hepatitis B:0.72), (CEPHALEXIN:Chronic hepatitis B:0.47), (CERTOLIZUMAB PEGOL:Chronic hepatitis B:0.04), (CETUXIMAB:Chronic hepatitis B:0.18), (CLOTRIMAZOLE:Chronic hepatitis B:0.01), (COBICISTAT\ELVITEGRAVIR\EMTRICITABINE\TENOFOVIR ALAFENAMIDE FUMARATE:Chronic hepatitis B:0), (CRIZOTINIB:Chronic hepatitis B:0.67), (DARUNAVIR:Chronic hepatitis B:0.2), (DESLORATADINE:Chronic hepatitis B:0.08), (DEXLANSOPRAZOLE:Chronic hepatitis B:0.22), (DICLOFENAC:Chronic hepatitis B:0.39), (DILTIAZEM:Chronic hepatitis B:0.75), (DILTIAZEM HYDROCHLORIDE:Chronic hepatitis B:0.66), (DIPHENHYDRAMINE:Chronic hepatitis B:0.07), (DIVALPROEX SODIUM:Chronic hepatitis B:0.72), (ENOXAPARIN:Chronic hepatitis B:0.6), (EPIRUBICIN:Chronic hepatitis B:0.04), (EVOLOCUMAB:Chronic hepatitis B:0.31), (FERROUS SULFATE\FERROUS SULFATE, DRIED:Chronic hepatitis B:0.33), (FLUTICASONE PROPIONATE:Chronic hepatitis B:0.15), (GLIPIZIDE:Chronic hepatitis B:0.08), (LEUCOVORIN CALCIUM:Chronic hepatitis B:0.24), (LITHIUM CARBONATE:Chronic hepatitis B:0.37), (NEBIVOLOL:Chronic hepatitis B:0.13), (OXCARBAZEPINE:Chronic hepatitis B:0.77), (OXYGEN:Chronic hepatitis B:0.04), (PERINDOPRIL:Chronic hepatitis B:0.18), (PHENYTOIN:Chronic hepatitis B:0.17), (SECUKINUMAB:Chronic hepatitis B:0.42), (TIOTROPIUM BROMIDE MONOHYDRATE:Chronic hepatitis B:0.08), (VALGANCICLOVIR HYDROCHLORIDE:Chronic hepatitis B:0.18), (.ALPHA.1-PROTEINASE INHIBITOR HUMAN:Chronic hepatitis C:0.61), (ABACAVIR\LAMIVUDINE:Chronic hepatitis C:0.76), (ACETAMINOPHEN\APRONALIDE\CAFFEINE\PROPYPHENAZONE:Chronic hepatitis C:0.39), (ACETAMINOPHEN\BROMISOVAL\DIHYDROCODEINE PHOSPHATE\DIPHENHYDRAMINE SALICYLATE\DYPHYLLINE\METHYLEPHEDRINE HYDROCHLORIDE, (+/−)-:Chronic hepatitis C:0), (ACETAMINOPHEN\CAFFEINE:Chronic hepatitis C:0.36), (ACETAMINOPHEN\PROPOXYPHENE HYDROCHLORIDE:Chronic hepatitis C:0.38), (ADENINE:Chronic hepatitis C:0.87), (ADO-TRASTUZUMAB EMTANSINE:Chronic hepatitis C:0.31), (AGALSIDASE BETA:Chronic hepatitis C:0.94), (ALEMTUZUMAB:Chronic hepatitis C:0.18), (ALISKIREN HEMIFUMARATE:Chronic hepatitis C:0.57), (ALOGLIPTIN:Chronic hepatitis C:0.26), (ALUMINUM SILICATE:Chronic hepatitis C:0.46), (AMIKACIN:Chronic hepatitis C:0.02), (AMINO ACIDS\CARBOHYDRATES\MINERALS\SOYBEAN OIL\VITAMINS:Chronic hepatitis C:0.3), (AMLODIPINE BESYLATE\HYDROCHLOROTHIAZIDE\VALSARTAN:Chronic hepatitis C:1.17), (AMOXICILLIN\CLARITHROMYCIN\ESOMEPRAZOLE:Chronic hepatitis C:0.33), (ANAGLIPTIN:Chronic hepatitis C:0.91), (ANAGRELIDE HYDROCHLORIDE:Chronic hepatitis C:0.05), (ANTIHISTAMINES NOS:Chronic hepatitis C:0.18), (APREPITANT:Chronic hepatitis C:0.14), (APRINDINE HYDROCHLORIDE:Chronic hepatitis C:0.31), (ASPIRIN\MAGNESIUM OXIDE:Chronic hepatitis C:0.52), (ATROPINE:Chronic hepatitis C:0), (AXITINIB:Chronic hepatitis C:0.68), (AZELASTINE HYDROCHLORIDE:Chronic hepatitis C:0.4), (BELATACEPT:Chronic hepatitis C:0.26), (BEPRIDIL:Chronic hepatitis C:1.46), (BERAPROST:Chronic hepatitis C:0.18), (BETAMETHASONE DIPROPIONATE\CALCIPOTRIENE\CALCIPOTRIENE HYDRATE:Chronic hepatitis C:0.63), (BETAMETHASONE SODIUM PHOSPHATE\NEOMYCIN SULFATE:Chronic hepatitis C:0.59), (BETAMETHASONE\CALCIPOTRIENE:Chronic hepatitis C:0.48), (BICARBONATE ION:Chronic hepatitis C:0.66), (BILASTINE:Chronic hepatitis C:0.66), (BIXALOMER:Chronic hepatitis C:0.12), (BOCOCIZUMAB:Chronic hepatitis C:0.01), (BUFORMIN:Chronic hepatitis C:0.02), (CALCITONIN:Chronic hepatitis C:0.44), (CALCITONIN SALMON:Chronic hepatitis C:0.28), (CALCIUM CARBONATE\ERGOCALCIFEROL\RETINOL:Chronic hepatitis C:0.22), (CALCIUM CHLORIDE\DEXTROSE\POTASSIUM CHLORIDE\SODIUM ACETATE\SODIUM CHLORIDE:Chronic hepatitis C:0.51), (CALCIUM\MAGNESIUM:Chronic hepatitis C:0.51), (CALCIUM\MAGNESIUM\ZINC:Chronic hepatitis C:0.76), (CAMPHOR\MENTHOL\METHYL SALICYLATE:Chronic hepatitis C:0.54), (CANAGLIFLOZIN\METFORMIN HYDROCHLORIDE:Chronic hepatitis C:1.12), (CANAKINUMAB:Chronic hepatitis C:0.88), (CARBIDOPA:Chronic hepatitis C:0.35), (CARBOMER:Chronic hepatitis C:0.22), (CARFILZOMIB:Chronic hepatitis C:0.72), (CARVEDILOL PHOSPHATE:Chronic hepatitis C:1.83), (CEFDITOREN PIVOXIL:Chronic hepatitis C:1.48), (CEFEPIME HYDROCHLORIDE:Chronic hepatitis C:0.46), (CEFOTAXIME\CEFOTAXIME SODIUM:Chronic hepatitis C:0.2), (CEFPODOXIME PROXETIL:Chronic hepatitis C:0.35), (CEFTERAM PIVOXIL:Chronic hepatitis C:0.2), (CEFUROXIME AXETIL:Chronic hepatitis C:0.27), (CELIPROLOL:Chronic hepatitis C:0.14), (CEPHARANTHINE:Chronic hepatitis C:0.35), (CERTOLIZUMAB PEGOL:Chronic hepatitis C:2.23), (CETUXIMAB:Chronic hepatitis C:0.2), (CEVIMELINE HYDROCHLORIDE:Chronic hepatitis C:0.02), (CHLORAMPHENICOL:Chronic hepatitis C:0.51), (CHLORPHENIRAMINE\HYDROCODONE:Chronic hepatitis C:0.13), (CHONDROITIN SULFATE (BOVINE)\GLUCOSAMINE HYDROCHLORIDE:Chronic hepatitis C:0.4), (CIFENLINE:Chronic hepatitis C:0.78), (CIFENLINE SUCCINATE:Chronic hepatitis C:0.14), (CIPROFIBRATE:Chronic hepatitis C:0.46), (CLIKSTAR:Chronic hepatitis C:0.04), (CLINDAMYCIN HYDROCHLORIDE:Chronic hepatitis C:0.07), (CLOBAZAM:Chronic hepatitis C:0.01), (CLOBETASONE BUTYRATE:Chronic hepatitis C:0.01), (CLOPERASTINE:Chronic hepatitis C:0.43), (COLESTILAN CHLORIDE:Chronic hepatitis C:0.29), (CORTICOTROPIN:Chronic hepatitis C:0.62), (CRANBERRY:Chronic hepatitis C:0.18), (DACLIZUMAB:Chronic hepatitis C:1.08), (DALFAMPRIDINE:Chronic hepatitis C:0.52), (DALTEPARIN:Chronic hepatitis C:1.5), (DALTEPARIN SODIUM:Chronic hepatitis C:1.26), (DANAZOL:Chronic hepatitis C:1.1), (DANTROLENE SODIUM:Chronic hepatitis C:0.04), (DAPAGLIFLOZIN PROPANEDIOL\METFORMIN HYDROCHLORIDE:Chronic hepatitis C:0.06), (DEGARELIX:Chronic hepatitis C:0.67), (DESONIDE:Chronic hepatitis C:0.94), (DIFLUCORTOLONE VALERATE\LIDOCAINE:Chronic hepatitis C:0.02), (DIMEMORFAN PHOSPHATE:Chronic hepatitis C:0.17), (DISTIGMINE BROMIDE:Chronic hepatitis C:0.71), (DOBUTAMINE HYDROCHLORIDE:Chronic hepatitis C:0.21), (DORZOLAMIDE:Chronic hepatitis C:0.65), (DORZOLAMIDE HYDROCHLORIDE:Chronic hepatitis C:0.42), (DOXYLAMINE SUCCINATE:Chronic hepatitis C:0.02), (DUTASTERIDE\TAMSULOSIN HYDROCHLORIDE:Chronic hepatitis C:1.04), (ECONAZOLE:Chronic hepatitis C:1.61), (EDARAVONE:Chronic hepatitis C:0.44), (ELCATONIN:Chronic hepatitis C:0.26), (ELOTUZUMAB:Chronic hepatitis C:0.01), (ENALAPRIL MALEATE\LERCANIDIPINE HYDROCHLORIDE:Chronic hepatitis C:0.29), (ERYTHROMYCIN LACTOBIONATE:Chronic hepatitis C:0.36), (ESOMEPRAZOLE MAGNESIUM\NAPROXEN:Chronic hepatitis C:0.13), (ESTRADIOL\NORETHINDRONE ACETATE:Chronic hepatitis C:0.12), (ESTROGENS:Chronic hepatitis C:0.34), (ETRETINATE:Chronic hepatitis C:2.12), (EVOLOCUMAB:Chronic hepatitis C:1.78), (FERRIC HYDROXIDE:Chronic hepatitis C:0.6), (FEXOFENADINE\PSEUDOEPHEDRINE:Chronic hepatitis C:0.56), (FINGOLIMOD HYDROCHLORIDE:Chronic hepatitis C:0.15), (FISH OIL\OMEGA-3 FATTY ACIDS:Chronic hepatitis C:0.36), (FLUPHENAZINE:Chronic hepatitis C:0.03), (FLUVOXAMINE:Chronic hepatitis C:0.04), (FONDAPARINUX SODIUM:Chronic hepatitis C:0.24), (FUDOSTEINE:Chronic hepatitis C:0.12), (FULVESTRANT:Chronic hepatitis C:0.43), (GENTAMICIN:Chronic hepatitis C:0.76), (GLUTAMINE\SODIUM GUALENATE:Chronic hepatitis C:0.34), (GLYCERIN:Chronic hepatitis C:0.47), (GLYCOPYRROLATE:Chronic hepatitis C:0.66), (GLYCOPYRROLATE\INDACATEROL MALEATE:Chronic hepatitis C:0.27), (GUANFACINE HYDROCHLORIDE:Chronic hepatitis C:0.55), (HYDROCHLOROTHIAZIDE\LOSARTAN:Chronic hepatitis C:0.1), (IBRUTINIB:Chronic hepatitis C:0.38), (IDELALISIB:Chronic hepatitis C:0.07), (IFENPRODIL TARTRATE:Chronic hepatitis C:0.13), (ILOPROST:Chronic hepatitis C:0.55), (IMIGLUCERASE:Chronic hepatitis C:0.75), (IMIQUIMOD:Chronic hepatitis C:0.26), (IMMUNE GLOBULIN NOS:Chronic hepatitis C:0.11), (INFLIXIMAB-DYYB:Chronic hepatitis C:0.74), (IPRAGLIFLOZIN:Chronic hepatitis C:0.03), (IRINOTECAN HYDROCHLORIDE:Chronic hepatitis C:0.13), (ISONIAZID\PYRAZINAMIDE\RIFAMPIN:Chronic hepatitis C:0.11), (ISONIAZID\RIFAMPIN:Chronic hepatitis C:0.64), (ITOPRIDE:Chronic hepatitis C:0.41), (IXAZOMIB:Chronic hepatitis C:0.21), (LACTASE:Chronic hepatitis C:0.5), (LEVALBUTEROL:Chronic hepatitis C:0.05), (LEVODOPA:Chronic hepatitis C:0.02), (LEVOLEUCOVORIN CALCIUM:Chronic hepatitis C:0.12), (LEVOMEFOLATE CALCIUM\METHYLCOBALAMIN\PYRIDOXAL PHOSPHATE ANHYDROUS:Chronic hepatitis C:0.05), (LIOTHYRONINE:Chronic hepatitis C:0.15), (LOMITAPIDE MESYLATE:Chronic hepatitis C:0.61), (LULICONAZOLE:Chronic hepatitis C:0.49), (LYSOZYME HYDROCHLORIDE:Chronic hepatitis C:0.95), (MAGNESIUM ASPARTATE HYDROCHLORIDE:Chronic hepatitis C:0.46), (MEPENZOLATE BROMIDE:Chronic hepatitis C:0.22), (MEPERIDINE:Chronic hepatitis C:0.31), (MEPERIDINE HYDROCHLORIDE:Chronic hepatitis C:0.42), (MEPOLIZUMAB:Chronic hepatitis C:0.85), (METFORMIN HYDROCHLORIDE\SAXAGLIPTIN HYDROCHLORIDE:Chronic hepatitis C:0.12), (METHENAMINE HIPPURATE:Chronic hepatitis C:0.87), (METHENOLONE ENANTHATE:Chronic hepatitis C:1.02), (METHYLPHENIDATE:Chronic hepatitis C:0.35), (METHYLPREDNISOLONE ACETATE:Chronic hepatitis C:1.53), (METRELEPTIN:Chronic hepatitis C:0.13), (MIDAZOLAM\MIDAZOLAM HYDROCHLORIDE:Chronic hepatitis C:0.09), (MIRIPLATIN:Chronic hepatitis C:0.34), (MOEXIPRIL HYDROCHLORIDE:Chronic hepatitis C:0.31), (MORPHINE HYDROCHLORIDE:Chronic hepatitis C:0.14), (MORPHINE SULFATE\NALTREXONE HYDROCHLORIDE:Chronic hepatitis C:0.62), (NICARDIPINE HYDROCHLORIDE:Chronic hepatitis C:0.56), (NIRAPARIB:Chronic hepatitis C:0.11), (ORPHENADRINE:Chronic hepatitis C:0.16), (OSELTAMIVIR:Chronic hepatitis C:0.18), (OSPEMIFENE:Chronic hepatitis C:0.25), (OXYBUTYNIN\OXYBUTYNIN CHLORIDE:Chronic hepatitis C:0.11), (PACLITAXEL:Chronic hepatitis C:0.66), (PARICALCITOL:Chronic hepatitis C:0.73), (PATIROMER:Chronic hepatitis C:0.47), (PAZOPANIB HYDROCHLORIDE:Chronic hepatitis C:1.3), (PEMETREXED:Chronic hepatitis C:0.07), (PEMETREXED DISODIUM:Chronic hepatitis C:0.74), (PIRITRAMIDE:Chronic hepatitis C:0.24), (POLYSTYRENE:Chronic hepatitis C:0.63), (POLYSTYRENE SULFONIC ACID:Chronic hepatitis C:0.34), (PONATINIB:Chronic hepatitis C:0.54), (POTASSIUM CHLORIDE\SODIUM CHLORIDE\SODIUM LACTATE:Chronic hepatitis C:0.69), (PREDNISOLONE SODIUM SUCCINATE:Chronic hepatitis C:0.85), (PRIMIDONE:Chronic hepatitis C:1.41), (PRISTINAMYCIN:Chronic hepatitis C:0.71), (PROBUCOL:Chronic hepatitis C:0.59), (PROPAFENONE HYDROCHLORIDE:Chronic hepatitis C:0.75), (PSYLLIUM HUSK:Chronic hepatitis C:0.8), (PYRIDOXAL PHOSPHATE:Chronic hepatitis C:0.29), (QUINACRINE:Chronic hepatitis C:0.59), (QUINAPRIL:Chronic hepatitis C:0.29), (RAMUCIRUMAB:Chronic hepatitis C:0.07), (RIFABUTIN:Chronic hepatitis C:0.71), (ROFECOXIB:Chronic hepatitis C:0.05), (SACCHAROMYCES CEREVISIAE:Chronic hepatitis C:0.4), (SARPOGRELATE HYDROCHLORIDE:Chronic hepatitis C:0.9), (SECUKINUMAB:Chronic hepatitis C:1.57), (SERRAPEPTASE:Chronic hepatitis C:0.29), (SODIUM ALGINATE:Chronic hepatitis C:0.23), (SODIUM FLUORIDE:Chronic hepatitis C:0.92), (SODIUM PHOSPHATE, DIBASIC\SODIUM PHOSPHATE, MONOBASIC:Chronic hepatitis C:0.6), (SOFALCONE:Chronic hepatitis C:0.08), (STREPTOMYCIN SULFATE:Chronic hepatitis C:0.07), (SUNITINIB MALATE:Chronic hepatitis C:0.47), (TEDUGLUTIDE\WATER:Chronic hepatitis C:0.06), (TERBUTALINE:Chronic hepatitis C:0.85), (TERIFLUNOMIDE:Chronic hepatitis C:0.19), (TESTOSTERONE UNDECANOATE:Chronic hepatitis C:0.86), (THALIDOMIDE:Chronic hepatitis C:0.57), (THIOCTIC ACID:Chronic hepatitis C:0.14), (TIANEPTINE:Chronic hepatitis C:0.22), (TICLOPIDINE:Chronic hepatitis C:0.56), (TIMOLOL\TRAVOPROST:Chronic hepatitis C:0.1), (TOFISOPAM:Chronic hepatitis C:0.27), (TOLTERODINE:Chronic hepatitis C:0.22), (TRIHEXYPHENIDYL:Chronic hepatitis C:0.29), (TROSPIUM CHLORIDE:Chronic hepatitis C:1.04), (TROXIPIDE:Chronic hepatitis C:0.28), (UMECLIDINIUM BROMIDE:Chronic hepatitis C:0.46), (VANCOMYCIN HYDROCHLORIDE:Chronic hepatitis C:0.52), (VEMURAFENIB:Chronic hepatitis C:0.13), (VONOPRAZAN:Chronic hepatitis C:0.39), (ZAFIRLUKAST:Chronic hepatitis C:0.35), (ZILEUTON:Chronic hepatitis C:0.21), (AMIODARONE HYDROCHLORIDE:Chronic inflammatory demyelinating polyradiculoneuropathy:0.56), (AZITHROMYCIN DIHYDRATE:Chronic inflammatory demyelinating polyradiculoneuropathy:0.07), (BUMETANIDE:Chronic inflammatory demyelinating polyradiculoneuropathy:0.72), (BUSPIRONE HYDROCHLORIDE:Chronic inflammatory demyelinating polyradiculoneuropathy:0.7), (CANDESARTAN:Chronic inflammatory demyelinating polyradiculoneuropathy:0.25), (CANDESARTAN CILEXETIL:Chronic inflammatory demyelinating polyradiculoneuropathy:0.38), (CERTOLIZUMAB PEGOL:Chronic inflammatory demyelinating polyradiculoneuropathy:0.91), (CHOLESTYRAMINE:Chronic inflammatory demyelinating polyradiculoneuropathy:0.3), (CLINDAMYCIN\CLINDAMYCIN PHOSPHATE:Chronic inflammatory demyelinating polyradiculoneuropathy:0.7), (COLCHICINE:Chronic inflammatory demyelinating polyradiculoneuropathy:0.19), (DACLIZUMAB:Chronic inflammatory demyelinating polyradiculoneuropathy:0.18), (DALFAMPRIDINE:Chronic inflammatory demyelinating polyradiculoneuropathy:0.81), (DASATINIB:Chronic inflammatory demyelinating polyradiculoneuropathy:0.02), (ELETRIPTAN HYDROBROMIDE:Chronic inflammatory demyelinating polyradiculoneuropathy:0.17), (ENALAPRIL MALEATE:Chronic inflammatory demyelinating polyradiculoneuropathy:0.1), (ESZOPICLONE:Chronic inflammatory demyelinating polyradiculoneuropathy:0.74), (EVEROLIMUS:Chronic inflammatory demyelinating polyradiculoneuropathy:0.08), (EVOLOCUMAB:Chronic inflammatory demyelinating polyradiculoneuropathy:0.56), (FESOTERODINE FUMARATE:Chronic inflammatory demyelinating polyradiculoneuropathy:0.13), (FLECAINIDE:Chronic inflammatory demyelinating polyradiculoneuropathy:0.25), (FLUOXETINE\FLUOXETINE HYDROCHLORIDE:Chronic inflammatory demyelinating polyradiculoneuropathy:0.06), (HYDRALAZINE HYDROCHLORIDE:Chronic inflammatory demyelinating polyradiculoneuropathy:0.2), (HYDROMORPHONE HYDROCHLORIDE:Chronic inflammatory demyelinating polyradiculoneuropathy:0.31), (ILOPROST:Chronic inflammatory demyelinating polyradiculoneuropathy:0.07), (IPRATROPIUM:Chronic inflammatory demyelinating polyradiculoneuropathy:0.17), (ISOSORBIDE:Chronic inflammatory demyelinating polyradiculoneuropathy:0.07), (ISOSORBIDE MONONITRATE:Chronic inflammatory demyelinating polyradiculoneuropathy:0.32), (LEDIPASVIR\SOFOSBUVIR:Chronic inflammatory demyelinating polyradiculoneuropathy:0.09), (LOVASTATIN:Chronic inflammatory demyelinating polyradiculoneuropathy:0.39), (NILOTINIB:Chronic inflammatory demyelinating polyradiculoneuropathy:0.08), (NITROGLYCERIN:Chronic inflammatory demyelinating polyradiculoneuropathy:0.37), (POLYETHYLENE GLYCOLS:Chronic inflammatory demyelinating polyradiculoneuropathy:0.46), (QUETIAPINE:Chronic inflammatory demyelinating polyradiculoneuropathy:0.05), (RIZATRIPTAN BENZOATE:Chronic inflammatory demyelinating polyradiculoneuropathy:0.07), (TERIFLUNOMIDE:Chronic inflammatory demyelinating polyradiculoneuropathy:0.56), (THALIDOMIDE:Chronic inflammatory demyelinating polyradiculoneuropathy:0.17), (TOFACITINIB CITRATE:Chronic inflammatory demyelinating polyradiculoneuropathy:0.02), (UMECLIDINIUM BROMIDE\VILANTEROL TRIFENATATE:Chronic inflammatory demyelinating polyradiculoneuropathy:0.36), (ACETAMINOPHEN\BUTALBITAL\CAFFEINE:Chronic kidney disease:0.83), (ACETAMINOPHEN\OXYCODONE\OXYCODONE HYDROCHLORIDE:Chronic kidney disease:0.49), (ACLIDINIUM BROMIDE:Chronic kidney disease:0.13), (ADO-TRASTUZUMAB EMTANSINE:Chronic kidney disease:0.31), (AGALSIDASE BETA:Chronic kidney disease:0.95), (ALEMTUZUMAB:Chronic kidney disease:0.19), (ALFUZOSIN HYDROCHLORIDE:Chronic kidney disease:0.06), (ALISKIREN HEMIFUMARATE:Chronic kidney disease:0.15), (AMANTADINE HYDROCHLORIDE:Chronic kidney disease:0.12), (AMBROXOL\CLENBUTEROL:Chronic kidney disease:0.05), (AMIKACIN:Chronic kidney disease:0.25), (AMILORIDE HYDROCHLORIDE\HYDROCHLOROTHIAZIDE:Chronic kidney disease:0.74), (AMISULPRIDE:Chronic kidney disease:0.57), (AMLODIPINE BESYLATE\ATORVASTATIN CALCIUM:Chronic kidney disease:0.39), (AMLODIPINE BESYLATE\HYDROCHLOROTHIAZIDE\VALSARTAN:Chronic kidney disease:0.46), (AMLODIPINE\PERINDOPRIL:Chronic kidney disease:0.31), (AMLODIPINE\VALSARTAN:Chronic kidney disease:0.56), (APREPITANT:Chronic kidney disease:0.25),(ARMODAFINIL:Chronic kidney disease:0.72), (ASPIRIN\CLOPIDOGREL:Chronic kidney disease:0), (ASPIRIN\MAGNESIUM OXIDE:Chronic kidney disease:0.19), (AZELASTINE:Chronic kidney disease:0.11), (BENAZEPRIL\BENAZEPRIL HYDROCHLORIDE:Chronic kidney disease:0.89), (BENIDIPINE:Chronic kidney disease:0.16), (BUCILLAMINE:Chronic kidney disease:0.08), (BUPRENORPHINE HYDROCHLORIDE\NALOXONE HYDROCHLORIDE:Chronic kidney disease:0.52), (CALCITONIN:Chronic kidney disease:0.32), (CALCITONIN SALMON:Chronic kidney disease:0.2), (CALCIUM CARBONATE\VITAMIN D:Chronic kidney disease:0.19), (CALCIUM CHLORIDE\MAGNESIUM CHLORIDE\SODIUM CHLORIDE\SODIUM LACTATE:Chronic kidney disease:0.13), (CANAGLIFLOZIN:Chronic kidney disease:0.94), (CANAGLIFLOZIN\METFORMIN HYDROCHLORIDE:Chronic kidney disease:0.63), (CANAKINUMAB:Chronic kidney disease:0.68), (CARBAZOCHROME SODIUM SULFONATE:Chronic kidney disease:0.06), (CARVEDILOL PHOSPHATE:Chronic kidney disease:1.42), (CASPOFUNGIN:Chronic kidney disease:0.26), (CEFUROXIME:Chronic kidney disease:1.1), (CHLORHEXIDINE:Chronic kidney disease:0.05), (CHLOROTHIAZIDE:Chronic kidney disease:0.35), (CHOLESTYRAMINE:Chronic kidney disease:1.76), (CICLESONIDE:Chronic kidney disease:0.21), (CIFENLINE:Chronic kidney disease:0.3), (CODEINE:Chronic kidney disease:0.48), (CRIZOTINIB:Chronic kidney disease:0.27), (CYPROHEPTADINE:Chronic kidney disease:0.26), (DABIGATRAN:Chronic kidney disease:1.41), (DACLIZUMAB:Chronic kidney disease:0.33), (DALFAMPRIDINE:Chronic kidney disease:0.32), (DANAZOL:Chronic kidney disease:1.2), (DAPAGLIFLOZIN PROPANEDIOL:Chronic kidney disease:0.58), (DEFERIPRONE:Chronic kidney disease:0.35), (DEFLAZACORT:Chronic kidney disease:0.82), (DESLORATADINE:Chronic kidney disease:0.85), (DESONIDE:Chronic kidney disease:0.17), (DIAZOXIDE:Chronic kidney disease:0.02), (DIPYRONE:Chronic kidney disease:0.83), (DOFETILIDE:Chronic kidney disease:1.1), (DOXEPIN HYDROCHLORIDE:Chronic kidney disease:0.23), (DRONABINOL:Chronic kidney disease:0.65), (ECONAZOLE:Chronic kidney disease:0.84), (EDOXABAN TOSILATE MONOHYDRATE:Chronic kidney disease:0.11), (EDOXABAN TOSYLATE:Chronic kidney disease:2.26), (ELOTUZUMAB:Chronic kidney disease:0.48), (EMPAGLIFLOZIN\LINAGLIPTIN:Chronic kidney disease:0.27), (ENALAPRIL MALEATE\HYDROCHLOROTHIAZIDE:Chronic kidney disease:0.14), (EPIRUBICIN:Chronic kidney disease:0.34), (ESTROGENS, CONJUGATED\MEDROXYPROGESTERONE ACETATE:Chronic kidney disease:0.17), (ETRETINATE:Chronic kidney disease:0.44), (FENOTEROL HYDROBROMIDE\IPRATROPIUM BROMIDE:Chronic kidney disease:0.23), (FESOTERODINE FUMARATE:Chronic kidney disease:1), (FINGOLIMOD HYDROCHLORIDE:Chronic kidney disease:0.05), (FONDAPARINUX:Chronic kidney disease:0.05), (FULVESTRANT:Chronic kidney disease:0.32), (FUSIDIC ACID:Chronic kidney disease:0.75), (GLIMEPIRIDE\ROSIGLITAZONE MALEATE:Chronic kidney disease:0.26), (HYDROCHLOROTHIAZIDE\IRBESARTAN:Chronic kidney disease:1.29), (HYDROCHLOROTHIAZIDE\TELMISARTAN:Chronic kidney disease:0.91), (HYDROXYCHLOROQUINE DIPHOSPHATE:Chronic kidney disease:0.29), (HYDROXYZINE PAMOATE:Chronic kidney disease:1.06), (HYOSCYAMINE:Chronic kidney disease:0.17), (IDARUCIZUMAB:Chronic kidney disease:0.22), (IMMUNE GLOBULIN NOS:Chronic kidney disease:0.18), (INDACATEROL:Chronic kidney disease:0.16), (INDAPAMIDE\PERINDOPRIL:Chronic kidney disease:1.65), (INTERFERON:Chronic kidney disease:0.28), (IPRAGLIFLOZIN L-PROLINE:Chronic kidney disease:0.01), (IRINOTECAN HYDROCHLORIDE:Chronic kidney disease:0.07), (IRSOGLADINE MALEATE:Chronic kidney disease:0.07), (ITRACONAZOLE:Chronic kidney disease:0.76), (LEVALBUTEROL:Chronic kidney disease:0.08), (MEGESTROL:Chronic kidney disease:0.06), (MEPERIDINE:Chronic kidney disease:0), (MEPERIDINE HYDROCHLORIDE:Chronic kidney disease:0.04), (METAXALONE:Chronic kidney disease:0.55), (METFORMIN HYDROCHLORIDE\ROSIGLITAZONE MALEATE:Chronic kidney disease:0.49), (METFORMIN HYDROCHLORIDE\VILDAGLIPTIN:Chronic kidney disease:0.64), (METHENAMINE HIPPURATE:Chronic kidney disease:0.49), (METHYLPREDNISOLONE ACETATE:Chronic kidney disease:0.7), (MINOCYCLINE HYDROCHLORIDE:Chronic kidney disease:0.23), (MINOCYCLINE\MINOCYCLINE HYDROCHLORIDE:Chronic kidney disease:0.84), (MODAFINIL:Chronic kidney disease:0.38), (NABUMETONE:Chronic kidney disease:0.34), (NADOLOL:Chronic kidney disease:0.99), (NIRAPARIB:Chronic kidney disease:0.14), (OLODATEROL HYDROCHLORIDE\TIOTROPIUM BROMIDE MONOHYDRATE:Chronic kidney disease:0.27), (OMEPRAZOLE SODIUM:Chronic kidney disease:0.8), (OXCARBAZEPINE:Chronic kidney disease:2.01), (PAROXETINE\PAROXETINE HYDROCHLORIDE:Chronic kidney disease:0.84), (PEGINTERFERON ALFA-2B:Chronic kidney disease:0.03), (PEGINTERFERON BETA-1A:Chronic kidney disease:0.8), (PEMETREXED DISODIUM:Chronic kidney disease:0.19), (PENTAMIDINE ISETHIONATE:Chronic kidney disease:0.19), (PILSICAINIDE HYDROCHLORIDE:Chronic kidney disease:1.31), (PIROXICAM:Chronic kidney disease:0.47), (POLYSTYRENE SULFONIC ACID:Chronic kidney disease:0.02), (PRISTINAMYCIN:Chronic kidney disease:0.24), (PROPAFENONE HYDROCHLORIDE:Chronic kidney disease:0.64), (PYRIDOSTIGMINE BROMIDE:Chronic kidney disease:0.31), (REGORAFENIB:Chronic kidney disease:0.06), (RETINOL:Chronic kidney disease:0.01), (RIOCIGUAT:Chronic kidney disease:0.02), (RIZATRIPTAN BENZOATE:Chronic kidney disease:0.49), (ROFLUMILAST:Chronic kidney disease:0.83), (ROSIGLITAZONE MALEATE:Chronic kidney disease:0.61), (SECUKINUMAB:Chronic kidney disease:0.97), (SODIUM FLUORIDE:Chronic kidney disease:0.25), (SODIUM OXYBATE:Chronic kidney disease:0.42), (SODIUM PHOSPHATE, DIBASIC\SODIUM PHOSPHATE, MONOBASIC:Chronic kidney disease:0.86), (SOTALOL:Chronic kidney disease:2.08), (SUMATRIPTAN:Chronic kidney disease:0.5), (SUMATRIPTAN\SUMATRIPTAN SUCCINATE:Chronic kidney disease:0.54), (TAZOBACTAM:Chronic kidney disease:0.14), (TEDUGLUTIDE\WATER:Chronic kidney disease:0.28), (TERBINAFINE HYDROCHLORIDE:Chronic kidney disease:0.26), (TERBUTALINE:Chronic kidney disease:0.24), (TERIFLUNOMIDE:Chronic kidney disease:0.11), (THYROID, PORCINE:Chronic kidney disease:0.14), (TIGECYCLINE:Chronic kidney disease:0.16), (TINZAPARIN:Chronic kidney disease:0.18), (TINZAPARIN SODIUM:Chronic kidney disease:0.73), (TIOTROPIUM BROMIDE:Chronic kidney disease:0.03), (TRANDOLAPRIL:Chronic kidney disease:0.05), (TRIAMCINOLONE ACETONIDE:Chronic kidney disease:0.27), (TRIAMTERENE:Chronic kidney disease:0.02), (UMECLIDINIUM BROMIDE:Chronic kidney disease:0.03), (VARENICLINE TARTRATE:Chronic kidney disease:0.87), (.ALPHA.1-PROTEINASE INHIBITOR HUMAN:Chronic lymphocytic leukaemia:0.73), (ABOBOTULINUMTOXINA:Chronic lymphocytic leukaemia:0.25), (ACECLOFENAC:Chronic lymphocytic leukaemia:0.05), (ACETAMINOPHEN\CAFFEINE\OPIUM:Chronic lymphocytic leukaemia:1.11), (ADO-TRASTUZUMAB EMTANSINE:Chronic lymphocytic leukaemia:1.14), (AGALSIDASE BETA:Chronic lymphocytic leukaemia:1.65), (ALBUTEROL\IPRATROPIUM BROMIDE:Chronic lymphocytic leukaemia:0.02), (ALCOHOL:Chronic lymphocytic leukaemia:0.82), (ALDESLEUKIN:Chronic lymphocytic leukaemia:0.07), (AMINO ACIDS\DEXTROSE\ELECTROLYTES NOS:Chronic lymphocytic leukaemia:0.87), (AMINOSALICYLIC ACID:Chronic lymphocytic leukaemia:0.11), (AMISULPRIDE:Chronic lymphocytic leukaemia:0.88), (AMLODIPINE BESYLATE\TELMISARTAN:Chronic lymphocytic leukaemia:0.12), (AMPICILLIN SODIUM:Chronic lymphocytic leukaemia:0.11), (ANAGRELIDE HYDROCHLORIDE:Chronic lymphocytic leukaemia:0.48), (ANHYDROUS CITRIC ACID\ASPIRIN\SODIUM BICARBONATE:Chronic lymphocytic leukaemia:0.01), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Chronic lymphocytic leukaemia:0.13), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Chronic lymphocytic leukaemia:0.28), (ASPIRIN\DIHYDROXYALUMINUM AMINOACETATE ANHYDROUS\MAGNESIUM CARBONATE:Chronic lymphocytic leukaemia:0.57), (AVANAFIL:Chronic lymphocytic leukaemia:0.07), (AZITHROMYCIN OR METHOTREXATE SODIUM:Chronic lymphocytic leukaemia:0.76), (BAMIFYLLINE HYDROCHLORIDE:Chronic lymphocytic leukaemia:0.14), (BELATACEPT:Chronic lymphocytic leukaemia:0.77), (BENSERAZIDE\LEVODOPA:Chronic lymphocytic leukaemia:0.06), (BETAMETHASONE DIPROPIONATE\CALCIPOTRIENE:Chronic lymphocytic leukaemia:0.5), (BETAMETHASONE DIPROPIONATE\CALCIPOTRIENE\CALCIPOTRIENE HYDRATE:Chronic lymphocytic leukaemia:0.74), (BETAMETHASONE\CALCIPOTRIENE:Chronic lymphocytic leukaemia:0.76), (BETHANECHOL CHLORIDE:Chronic lymphocytic leukaemia:0.32), (BOCOCIZUMAB:Chronic lymphocytic leukaemia:0.18), (BOTULINUM TOXIN TYPE A:Chronic lymphocytic leukaemia:0.59), (BUPIVACAINE HYDROCHLORIDE:Chronic lymphocytic leukaemia:0.24), (BUPRENORPHINE HYDROCHLORIDE:Chronic lymphocytic leukaemia:1.18), (BUPRENORPHINE HYDROCHLORIDE\NALOXONE HYDROCHLORIDE:Chronic lymphocytic leukaemia:0.34), (CABAZITAXEL:Chronic lymphocytic leukaemia:0.09), (CALCIUM CARBONATE\ERGOCALCIFEROL:Chronic lymphocytic leukaemia:0.02), (CALCIUM CARBONATE\ERGOCALCIFEROL\RETINOL:Chronic lymphocytic leukaemia:0.36), (CALCIUM CITRATE\VITAMIN D:Chronic lymphocytic leukaemia:0.3), (CALCIUM PANTOTHENATE\CYANOCOBALAMIN\NIACINAMIDE\PYRIDOXINE HYDROCHLORIDE\RIBOFLAVIN\THIAMINE MONONITRATE:Chronic lymphocytic leukaemia:0.84), (CALCIUM\MAGNESIUM\ZINC:Chronic lymphocytic leukaemia:0.81), (CALCIUM\VITAMINS:Chronic lymphocytic leukaemia:0.09), (CANAGLIFLOZIN\METFORMIN HYDROCHLORIDE:Chronic lymphocytic leukaemia:1.1), (CANAKINUMAB:Chronic lymphocytic leukaemia:1.23), (CAPSAICIN:Chronic lymphocytic leukaemia:0.6), (CARFILZOMIB:Chronic lymphocytic leukaemia:1.84), (CARTEOLOL:Chronic lymphocytic leukaemia:0.63), (CARTEOLOL HYDROCHLORIDE:Chronic lymphocytic leukaemia:0.03), (CEFOTAXIME\CEFOTAXIME SODIUM:Chronic lymphocytic leukaemia:0.67), (CEFPROZIL:Chronic lymphocytic leukaemia:0.3), (CERITINIB:Chronic lymphocytic leukaemia:0.4), (CERTOLIZUMAB PEGOL:Chronic lymphocytic leukaemia:1.5), (CHLOROTHIAZIDE:Chronic lymphocytic leukaemia:0.19), (CHOLESTEROL:Chronic lymphocytic leukaemia:0.1), (CHONDROITIN SULFATE (CHICKEN):Chronic lymphocytic leukaemia:0.81), (CLIKSTAR:Chronic lymphocytic leukaemia:0.56), (CLOMIPRAMINE HYDROCHLORIDE:Chronic lymphocytic leukaemia:0.95), (CORTICOTROPIN:Chronic lymphocytic leukaemia:0.91), (CRIZOTINIB:Chronic lymphocytic leukaemia:0.35), (CYPROTERONE ACETATE:Chronic lymphocytic leukaemia:0.04), (DACLIZUMAB:Chronic lymphocytic leukaemia:2.14), (DALFAMPRIDINE:Chronic lymphocytic leukaemia:0.88), (DANTROLENE SODIUM:Chronic lymphocytic leukaemia:0.38), (DARATUMUMAB:Chronic lymphocytic leukaemia:0.55), (DASABUVIR\OMBITASVIR\PARITAPREVIR\RITONAVIR:Chronic lymphocytic leukaemia:0.3), (DEFLAZACORT:Chronic lymphocytic leukaemia:1.38), (DESFLURANE:Chronic lymphocytic leukaemia:0.42), (DEVICE:Chronic lymphocytic leukaemia:0.41), (DEXAMETHASONE ACETATE:Chronic lymphocytic leukaemia:0.86), (DEXTROAMPHETAMINE:Chronic lymphocytic leukaemia:0.21), (DEXTROAMPHETAMINE SULFATE:Chronic lymphocytic leukaemia:0.71), (DICLOFENAC SODIUM\MISOPROSTOL:Chronic lymphocytic leukaemia:0.29), (DIFLUNISAL:Chronic lymphocytic leukaemia:0.29), (DIPYRONE\PROMETHAZINE HYDROCHLORIDE:Chronic lymphocytic leukaemia:0.42), (DOPAMINE\DOPAMINE HYDROCHLORIDE:Chronic lymphocytic leukaemia:0.05), (DROXIDOPA:Chronic lymphocytic leukaemia:0.13), (ELETRIPTAN HYDROBROMIDE:Chronic lymphocytic leukaemia:2.13), (ELOTUZUMAB:Chronic lymphocytic leukaemia:1.33), (EMPAGLIFLOZIN\LINAGLIPTIN:Chronic lymphocytic leukaemia:0.64), (ESMOLOL HYDROCHLORIDE:Chronic lymphocytic leukaemia:0.09), (ESOMEPRAZOLE MAGNESIUM\NAPROXEN:Chronic lymphocytic leukaemia:0.18), (ESOMEPRAZOLE SODIUM:Chronic lymphocytic leukaemia:0.27), (ESTROGENS:Chronic lymphocytic leukaemia:0.65), (ETHINYL ESTRADIOL\NORGESTREL:Chronic lymphocytic leukaemia:0.17), (EVOLOCUMAB:Chronic lymphocytic leukaemia:1.45), (FERROUS BISGLYCINATE:Chronic lymphocytic leukaemia:0), (FLUNARIZINE HYDROCHLORIDE:Chronic lymphocytic leukaemia:0.18), (FLUNISOLIDE:Chronic lymphocytic leukaemia:0.36), (FORMOTEROL FUMARATE\MOMETASONE FUROATE:Chronic lymphocytic leukaemia:1.11), (FOSFOMYCIN:Chronic lymphocytic leukaemia:1.12), (GIMERACIL\OTERACIL\TEGAFUR:Chronic lymphocytic leukaemia:0), (GLUTAMINE:Chronic lymphocytic leukaemia:0.28), (GLYCERIN\HYPROMELLOSES\POLYETHYLENE GLYCOL 400:Chronic lymphocytic leukaemia:0.45), (GLYCOPYRROLATE\INDACATEROL MALEATE:Chronic lymphocytic leukaemia:0.78), (IBRITUMOMAB TIUXETAN:Chronic lymphocytic leukaemia:0.83), (IDARUCIZUMAB:Chronic lymphocytic leukaemia:0.09), (ILOPROST:Chronic lymphocytic leukaemia:0.47), (IMIGLUCERASE:Chronic lymphocytic leukaemia:0.47), (INFLIXIMAB-DYYB:Chronic lymphocytic leukaemia:0.42), (INOTUZUMAB OZOGAMICIN:Chronic lymphocytic leukaemia:0.53), (INSULIN PORKINSULIN PURIFIED PORK:Chronic lymphocytic leukaemia:0.11), (INTERFERON:Chronic lymphocytic leukaemia:0.79), (INTERFERON ALFA-2A:Chronic lymphocytic leukaemia:1.33), (INTERFERON ALFA-2B:Chronic lymphocytic leukaemia:0.16), (INTERFERON GAMMA-1B:Chronic lymphocytic leukaemia:1.23), (IRINOTECAN HYDROCHLORIDE:Chronic lymphocytic leukaemia:0.86), (ISONIAZID\RIFAMPIN:Chronic lymphocytic leukaemia:0.19), (IVABRADINE HYDROCHLORIDE:Chronic lymphocytic leukaemia:0.42), (IVERMECTIN:Chronic lymphocytic leukaemia:0.45), (LACTASE:Chronic lymphocytic leukaemia:1.14), (LANREOTIDE:Chronic lymphocytic leukaemia:0.28), (LAPATINIB DITOSYLATE:Chronic lymphocytic leukaemia:0.38), (LENVATINIB:Chronic lymphocytic leukaemia:0), (LEVOLEUCOVORIN CALCIUM:Chronic lymphocytic leukaemia:0.27), (LEVOMILNACIPRAN HYDROCHLORIDE:Chronic lymphocytic leukaemia:0), (LOMITAPIDE MESYLATE:Chronic lymphocytic leukaemia:0.65), (LOMUSTINE:Chronic lymphocytic leukaemia:0.75), (MAGNESIUM PIDOLATE:Chronic lymphocytic leukaemia:0.18), (MEDROXYPROGESTERONE:Chronic lymphocytic leukaemia:0.5), (MELPERONE:Chronic lymphocytic leukaemia:0.87), (MEPOLIZUMAB:Chronic lymphocytic leukaemia:0.75), (METFORMIN HYDROCHLORIDE\ROSIGLITAZONE MALEATE:Chronic lymphocytic leukaemia:0.17), (METFORMIN HYDROCHLORIDE\SAXAGLIPTIN:Chronic lymphocytic leukaemia:0.38), (METHENOLONE ENANTHATE:Chronic lymphocytic leukaemia:0.38), (MIANSERIN:Chronic lymphocytic leukaemia:0.53), (NALBUPHINE HYDROCHLORIDE:Chronic lymphocytic leukaemia:0.01), (NINTEDANIB:Chronic lymphocytic leukaemia:0.88), (NIRAPARIB:Chronic lymphocytic leukaemia:0.41), (NOREPINEPHRINE BITARTRATE:Chronic lymphocytic leukaemia:0.65), (OLAPARIB:Chronic lymphocytic leukaemia:0.12), (OSIMERTINIB:Chronic lymphocytic leukaemia:0.1), (OXYBUTYNIN\OXYBUTYNIN CHLORIDE:Chronic lymphocytic leukaemia:0.55), (PATIROMER:Chronic lymphocytic leukaemia:0.74), (PEGASPARGASE:Chronic lymphocytic leukaemia:0.06), (PEGINTERFERON ALFA-2A:Chronic lymphocytic leukaemia:0.06), (PEGINTERFERON BETA-1A:Chronic lymphocytic leukaemia:0.64), (PEMETREXED:Chronic lymphocytic leukaemia:0.89), (PERTUZUMAB:Chronic lymphocytic leukaemia:0.43), (PILOCARPINE HYDROCHLORIDE:Chronic lymphocytic leukaemia:0.38), (PINDOLOL:Chronic lymphocytic leukaemia:0.08), (PIRACETAM:Chronic lymphocytic leukaemia:0.01), (PIRFENIDONE:Chronic lymphocytic leukaemia:0.32), (PIRITRAMIDE:Chronic lymphocytic leukaemia:0.61), (POLYETHYLENE GLYCOL 3350\POTASSIUM CHLORIDE\SODIUM BICARBONATE\SODIUM CHLORIDE\SODIUM SULFATE ANHYDROUS:Chronic lymphocytic leukaemia:0.56), (PORFIMER SODIUM:Chronic lymphocytic leukaemia:0.02), (PROTRIPTYLINE HYDROCHLORIDE:Chronic lymphocytic leukaemia:0.32), (PYRAZINAMIDE:Chronic lymphocytic leukaemia:0.75), (QUINACRINE:Chronic lymphocytic leukaemia:0.19), (RADIATION THERAPY:Chronic lymphocytic leukaemia:1.49), (RALTEGRAVIR POTASSIUM:Chronic lymphocytic leukaemia:0.18), (RIZATRIPTAN:Chronic lymphocytic leukaemia:0.59), (SACUBITRIL\VALSARTAN:Chronic lymphocytic leukaemia:0.72), (SECUKINUMAB:Chronic lymphocytic leukaemia:1.21), (SIPULEUCEL-T:Chronic lymphocytic leukaemia:0.37), (SODIUM CITRATE:Chronic lymphocytic leukaemia:0.46), (SOFOSBUVIR\VELPATASVIR:Chronic lymphocytic leukaemia:0.51), (SORBITOL:Chronic lymphocytic leukaemia:0.37), (TAFAMIDIS MEGLUMINE:Chronic lymphocytic leukaemia:0.11), (TEDUGLUTIDE\WATER:Chronic lymphocytic leukaemia:0.48), (TESTOSTERONE CYPIONATE:Chronic lymphocytic leukaemia:0.78), (TESTOSTERONE UNDECANOATE:Chronic lymphocytic leukaemia:0.89), (TETRAZEPAM:Chronic lymphocytic leukaemia:0.23), (TIMOLOL MALEATE\TRAVOPROST:Chronic lymphocytic leukaemia:0.54), (TIMOLOL\TRAVOPROST:Chronic lymphocytic leukaemia:0.19), (TRIMIPRAMINE MALEATE:Chronic lymphocytic leukaemia:1.09), (VALDECOXIB:Chronic lymphocytic leukaemia:0.5), (VASOPRESSIN:Chronic lymphocytic leukaemia:0.14), (VINBLASTINE SULFATE:Chronic lymphocytic leukaemia:0.27), (VINORELBINE TARTRATE:Chronic lymphocytic leukaemia:0.78), (XIPAMIDE:Chronic lymphocytic leukaemia:0.12), (YEAST:Chronic lymphocytic leukaemia:0.31), (ZAFIRLUKAST:Chronic lymphocytic leukaemia:1.11), (ZICONOTIDE ACETATE:Chronic lymphocytic leukaemia:0.04), (ZILEUTON:Chronic lymphocytic leukaemia:0.62), (ZIPRASIDONE HYDROCHLORIDE\ZIPRASIDONE MESYLATE:Chronic lymphocytic leukaemia:0.23), (ZIV-AFLIBERCEPT: Chronic lymphocytic leukaemia:0.13), (.ALPHA.1-PROTEINASE INHIBITOR HUMAN:Chronic myeloid leukaemia:0.35), (ABOBOTULINUMTOXINA:Chronic myeloid leukaemia:0.14), (ACETAMINOPHEN\CAFFEINE:Chronic myeloid leukaemia:0.47), (ACETAMINOPHEN\PROPOXYPHENE NAPSYLATE:Chronic myeloid leukaemia:0.45), (ACETAZOLAMIDE:Chronic myeloid leukaemia:2.84), (ACITRETIN:Chronic myeloid leukaemia:0.73), (ADENOSINE:Chronic myeloid leukaemia:0.02), (ADO-TRASTUZUMAB EMTANSINE:Chronic myeloid leukaemia:1.15), (AFATINIB:Chronic myeloid leukaemia:0.13), (AGALSIDASE BETA:Chronic myeloid leukaemia:1.2), (ALCOHOL:Chronic myeloid leukaemia:0.61), (ALDESLEUKIN:Chronic myeloid leukaemia:0.04), (AMINO ACIDS\DEXTROSE\ELECTROLYTES NOS:Chronic myeloid leukaemia:0.85), (APREPITANT:Chronic myeloid leukaemia:0.94), (ASUNAPREVIR:Chronic myeloid leukaemia:0.03), (ATROPINE:Chronic myeloid leukaemia:0.48), (ATROPINE SULFATE:Chronic myeloid leukaemia:0.33), (AXITINIB:Chronic myeloid leukaemia:1.13), (AZITHROMYCIN OR METHOTREXATE SODIUM:Chronic myeloid leukaemia:0.21), (BACITRACIN:Chronic myeloid leukaemia:0.62), (BALSALAZIDE DISODIUM:Chronic myeloid leukaemia:0.11), (BAMIFYLLINE HYDROCHLORIDE:Chronic myeloid leukaemia:0.04), (BECLOMETHASONE DIPROPIONATE\FORMOTEROL:Chronic myeloid leukaemia:0.34), (BELATACEPT:Chronic myeloid leukaemia:0.31), (BENDAMUSTINE HYDROCHLORIDE:Chronic myeloid leukaemia:0.72), (BENZTROPINE MESYLATE:Chronic myeloid leukaemia:0.02), (BEPRIDIL:Chronic myeloid leukaemia:0.88), (BERAPROST:Chronic myeloid leukaemia:0.34), (BETAMETHASONE DIPROPIONATE\CALCIPOTRIENE\CALCIPOTRIENE HYDRATE:Chronic myeloid leukaemia:0.64), (BETAMETHASONE\CALCIPOTRIENE:Chronic myeloid leukaemia:0.6), (BIFIDOBACTERIUM LONGUM SUBSP. INFANTIS:Chronic myeloid leukaemia:0.67), (BIMATOPROST\TIMOLOL:Chronic myeloid leukaemia:0.18), (BIPERIDEN HYDROCHLORIDE:Chronic myeloid leukaemia:0.17), (BOCEPREVIR:Chronic myeloid leukaemia:0.31), (BOCOCIZUMAB:Chronic myeloid leukaemia:0.47), (BRINZOLAMIDE\TIMOLOL MALEATE:Chronic myeloid leukaemia:0.18), (BUDESONIDE\FORMOTEROL FUMARATE:Chronic myeloid leukaemia:1.53), (CABAZITAXEL:Chronic myeloid leukaemia:0.24), (CALCIPOTRIENE:Chronic myeloid leukaemia:0.79), (CALCIUM CARBONATE\ERGOCALCIFEROL\RETINOL:Chronic myeloid leukaemia:0.61), (CALCIUM LACTATE:Chronic myeloid leukaemia:0.2), (CALCIUM PANTOTHENATE\CYANOCOBALAMIN\NIACINAMIDE\PYRIDOXINE HYDROCHLORIDE\RIBOFLAVIN\THIAMINE MONONITRATE:Chronic myeloid leukaemia:0.23), (CALCIUM POLYCARBOPHIL:Chronic myeloid leukaemia:0.59), (CALCIUM\MAGNESIUM:Chronic myeloid leukaemia:0.66), (CALCIUM\MAGNESIUM\ZINC:Chronic myeloid leukaemia:0.67), (CANAGLIFLOZIN\\METFORMIN HYDROCHLORIDE:Chronic myeloid leukaemia:1.31), (CARBIDOPA:Chronic myeloid leukaemia:0.48), (CARFILZOMIB:Chronic myeloid leukaemia:1.61), (CARTEOLOL:Chronic myeloid leukaemia:0.23), (CARVEDILOL PHOSPHATE:Chronic myeloid leukaemia:2.3), (CEFOTAXIME\CEFOTAXIME SODIUM:Chronic myeloid leukaemia:0.02), (CERTOLIZUMAB PEGOL:Chronic myeloid leukaemia:1.76), (CETIRIZINE HYDROCHLORIDE\PSEUDOEPHEDRINE HYDROCHLORIDE:Chronic myeloid leukaemia:0.39), (CHLOROTHIAZIDE:Chronic myeloid leukaemia:0.74), (CHLORPHENIRAMINE\HYDROCODONE:Chronic myeloid leukaemia:0.7), (CHLORPROMAZINE:Chronic myeloid leukaemia:0.52), (CLIKSTAR:Chronic myeloid leukaemia:0.27), (CLOMIPRAMINE HYDROCHLORIDE:Chronic myeloid leukaemia:0.9), (COLCHICINE\DICYCLOMINE HYDROCHLORIDE:Chronic myeloid leukaemia:0.11), (COLISTIMETHATE SODIUM:Chronic myeloid leukaemia:0.09), (COLISTIN:Chronic myeloid leukaemia:0), (CORTICOTROPIN:Chronic myeloid leukaemia:0.72), (CYPROHEPTADINE HYDROCHLORIDE:Chronic myeloid leukaemia:0.09), (DABIGATRAN ETEXILATE:Chronic myeloid leukaemia:0.09), (DABRAFENIB:Chronic myeloid leukaemia:0.55), (DACLIZUMAB:Chronic myeloid leukaemia:1.14), (DANTROLENE SODIUM:Chronic myeloid leukaemia:0.1), (DAPAGLIFLOZIN PROPANEDIOL\\METFORMIN HYDROCHLORIDE:Chronic myeloid leukaemia:0.09), (DARATUMUMAB:Chronic myeloid leukaemia:0.14), (DEFLAZACORT:Chronic myeloid leukaemia:1.24), (DEGARELIX:Chronic myeloid leukaemia:0.28), (DESOXIMETASONE:Chronic myeloid leukaemia:0.13), (DEVICE:Chronic myeloid leukaemia:0.68), (DEXAMETHASONE ACETATE:Chronic myeloid leukaemia:1.24), (DOLASETRON MESYLATE:Chronic myeloid leukaemia:0.32), (DOPAMINE HYDROCHLORIDE:Chronic myeloid leukaemia:0.02), (DOXYLAMINE SUCCINATE:Chronic myeloid leukaemia:0.38), (DROSPIRENONE\ETHINYL ESTRADIOL:Chronic myeloid leukaemia:0.34), (EDOXABAN TOSILATE MONOHYDRATE:Chronic myeloid leukaemia:0.38), (ELDECALCITOL:Chronic myeloid leukaemia:0.66), (ELOTUZUMAB:Chronic myeloid leukaemia:0.94), (EMPAGLIFLOZIN\LINAGLIPTIN:Chronic myeloid leukaemia:0.6), (ENALAPRIL MALEATE\HYDROCHLOROTHIAZIDE:Chronic myeloid leukaemia:0.8), (ERIBULIN:Chronic myeloid leukaemia:0.08), (ERTAPENEM SODIUM:Chronic myeloid leukaemia:0.28), (ESTRIOL:Chronic myeloid leukaemia:0.14), (ETHACRYNIC ACID:Chronic myeloid leukaemia:0.41), (ETOPOSIDE PHOSPHATE:Chronic myeloid leukaemia:0.44), (EVOLOCUMAB:Chronic myeloid leukaemia:1.72), (FENOTEROL HYDROBROMIDE\\IPRATROPIUM BROMIDE:Chronic myeloid leukaemia:1.2), (FERRIC HYDROXIDE:Chronic myeloid leukaemia:0.36), (FERROUS BISGLYCINATE:Chronic myeloid leukaemia:0.03), (FLUTICASONE FUROATE\VILANTEROL TRIFENATATE:Chronic myeloid leukaemia:1.35), (FONDAPARINUX:Chronic myeloid leukaemia:0.54), (FUSIDIC ACID:Chronic myeloid leukaemia:1.69), (GLIMEPIRIDE\ROSIGLITAZONE MALEATE:Chronic myeloid leukaemia:0.22), (GLIPIZIDE\METFORMIN HYDROCHLORIDE:Chronic myeloid leukaemia:0.07), (GLUTAMINE:Chronic myeloid leukaemia:0.01), (GLYCOPYRROLATE\INDACATEROL MALEATE:Chronic myeloid leukaemia:0.59), (HEPARIN CALCIUM:Chronic myeloid leukaemia:1.88), (HYDROCORTISONE ACETATE:Chronic myeloid leukaemia:0.44), (IBRITUMOMAB TIUXETAN:Chronic myeloid leukaemia:0.7), (ICOSAPENT ETHYL:Chronic myeloid leukaemia:0.5), (IDARUCIZUMAB:Chronic myeloid leukaemia:0.43), (IMIGLUCERASE:Chronic myeloid leukaemia:0.66), (INFLIXIMAB-DYYB:Chronic myeloid leukaemia:0.4), (INOTUZUMAB OZOGAMICIN:Chronic myeloid leukaemia:0.35), (INTERFERON GAMMA-1B:Chronic myeloid leukaemia:0.07), (IRINOTECAN:Chronic myeloid leukaemia:0.48), (IRINOTECAN HYDROCHLORIDE:Chronic myeloid leukaemia:0.69), (IRON\POLYSACCHARIDES:Chronic myeloid leukaemia:0.06), (ISONIAZID\RIFAMPIN:Chronic myeloid leukaemia:0.01), (IVABRADINE HYDROCHLORIDE:Chronic myeloid leukaemia:0.29), (LANREOTIDE:Chronic myeloid leukaemia:0.2), (LANREOTIDE ACETATE:Chronic myeloid leukaemia:0.61), (LENVATINIB:Chronic myeloid leukaemia:0.08), (LEVOLEUCOVORIN CALCIUM:Chronic myeloid leukaemia:0.41), (LEVOMEFOLATE CALCIUM:Chronic myeloid leukaemia:0.1), (LEVOSIMENDAN:Chronic myeloid leukaemia:0.07), (LIOTHYRONINE:Chronic myeloid leukaemia:0.36), (LISDEXAMFETAMINE DIMESYLATE:Chronic myeloid leukaemia:0.41), (LOMITAPIDE MESYLATE:Chronic myeloid leukaemia:0.58), (LORNOXICAM:Chronic myeloid leukaemia:0.04), (LURASIDONE HYDROCHLORIDE:Chronic myeloid leukaemia:0.01), (MAGNESIUM ASPARTATE\POTASSIUM ASPARTATE:Chronic myeloid leukaemia:0.32), (MAXACALCITOL:Chronic myeloid leukaemia:0.34), (MEPOLIZUMAB:Chronic myeloid leukaemia:0.44), (METFORMIN HYDROCHLORIDE\ROSIGLITAZONE MALEATE:Chronic myeloid leukaemia:0.47), (METFORMIN HYDROCHLORIDE\SAXAGLIPTIN HYDROCHLORIDE:Chronic myeloid leukaemia:0.36), (METHENOLONE ENANTHATE:Chronic myeloid leukaemia:0.9), (METRELEPTIN:Chronic myeloid leukaemia:0.34), (MEXILETINE:Chronic myeloid leukaemia:0.19), (MICONAZOLE NITRATE:Chronic myeloid leukaemia:0.2), (MIDAZOLAM HYDROCHLORIDE:Chronic myeloid leukaemia:0.33), (MIDODRINE HYDROCHLORIDE:Chronic myeloid leukaemia:0.81), (MIGLUSTAT:Chronic myeloid leukaemia:0.19), (MILRINONE:Chronic myeloid leukaemia:0.5), (MINODRONIC ACID:Chronic myeloid leukaemia:0.06), (MITIGLINIDE:Chronic myeloid leukaemia:0.16), (MIZORIBINE:Chronic myeloid leukaemia:0.27), (MORPHINE SULFATE\NALTREXONE HYDROCHLORIDE:Chronic myeloid leukaemia:0.59), (NALBUPHINE HYDROCHLORIDE:Chronic myeloid leukaemia:0.17), (NALOXONE:Chronic myeloid leukaemia:0.24), (NARATRIPTAN HYDROCHLORIDE:Chronic myeloid leukaemia:0.18), (NEPAFENAC:Chronic myeloid leukaemia:0.09), (NINTEDANIB:Chronic myeloid leukaemia:1.11), (NIRAPARIB:Chronic myeloid leukaemia:0.45), (NOREPINEPHRINE:Chronic myeloid leukaemia:0.69), (OBINUTUZUMAB:Chronic myeloid leukaemia:0.07), (OLAPARIB:Chronic myeloid leukaemia:0.22), (OSIMERTINIB:Chronic myeloid leukaemia:0.08), (OSPEMIFENE:Chronic myeloid leukaemia:0.25), (OXAPROZIN:Chronic myeloid leukaemia:0.18), (OXYBUTYNIN\OXYBUTYNIN CHLORIDE:Chronic myeloid leukaemia:0.18), (PAMIDRONATE DISODIUM:Chronic myeloid leukaemia:0.8), (PANITUMUMAB:Chronic myeloid leukaemia:0.18), (PASIREOTIDE:Chronic myeloid leukaemia:0.35), (PATIROMER:Chronic myeloid leukaemia:0.67), (PAZOPANIB:Chronic myeloid leukaemia:0.04), (PEGASPARGASE:Chronic myeloid leukaemia:0.6), (PEGINTERFERON BETA-1A:Chronic myeloid leukaemia:1.04), (PEMETREXED:Chronic myeloid leukaemia:0.93), (PENICILLIN V POTASSIUM:Chronic myeloid leukaemia:0.28), (PENTOSTATIN:Chronic myeloid leukaemia:0.68), (PILOCARPINE:Chronic myeloid leukaemia:0.67), (PIMECROLIMUS:Chronic myeloid leukaemia:0.07), (PIPERACILLIN:Chronic myeloid leukaemia:0.33), (PIRFENIDONE:Chronic myeloid leukaemia:0.15), (PNEUMOCOCCAL VACCINE, POLYVALENT 23:Chronic myeloid leukaemia:0.48), (POLAPREZINC:Chronic myeloid leukaemia:0.13), (PREDNISOLONE SODIUM SUCCINATE:Chronic myeloid leukaemia:0.11), (PROBUCOL:Chronic myeloid leukaemia:0.03), (PYRIDOXAL:Chronic myeloid leukaemia:0.28), (RAMUCIRUMAB:Chronic myeloid leukaemia:0.47), (RILPIVIRINE HYDROCHLORIDE:Chronic myeloid leukaemia:0.04), (RIVASTIGMINE:Chronic myeloid leukaemia:0.99), (SECUKINUMAB:Chronic myeloid leukaemia:1.49), (SELEGILINE HYDROCHLORIDE:Chronic myeloid leukaemia:0.19), (SELEXIPAG:Chronic myeloid leukaemia:0.36), (SEVELAMER CARBONATE:Chronic myeloid leukaemia:0.69), (SIPULEUCEL-T:Chronic myeloid leukaemia:0.1), (SODIUM OXYBATE:Chronic myeloid leukaemia:1.09), (SOFOSBUVIR\VELPATASVIR:Chronic myeloid leukaemia:0.26), (SUPLATAST TOSILATE:Chronic myeloid leukaemia:0.12), (TAFAMIDIS MEGLUMINE:Chronic myeloid leukaemia:0.2), (TAZOBACTAM:Chronic myeloid leukaemia:0.8), (TEDUGLUTIDE\WATER:Chronic myeloid leukaemia:0.36), (TELOTRISTAT ETHYL:Chronic myeloid leukaemia:0.12), (TEMSIROLIMUS:Chronic myeloid leukaemia:0.19), (TENELIGLIPTIN:Chronic myeloid leukaemia:0.24), (TESTOSTERONE UNDECANOATE:Chronic myeloid leukaemia:0.83), (THIOCTIC ACID:Chronic myeloid leukaemia:0.7), (TIGECYCLINE:Chronic myeloid leukaemia:0.51), (TIMOLOL MALEATE\TRAVOPROST:Chronic myeloid leukaemia:0.05), (TOLVAPTAN:Chronic myeloid leukaemia:0.33), (TRAMETINIB:Chronic myeloid leukaemia:0.33), (TREPROSTINIL DIOLAMINE:Chronic myeloid leukaemia:0.03), (TRIPTORELIN PAMOATE:Chronic myeloid leukaemia:0.12), (UMECLIDINIUM BROMIDE\VILANTEROL TRIFENATATE:Chronic myeloid leukaemia:1.06), (UROKINASE:Chronic myeloid leukaemia:0.01), (VEMURAFENIB:Chronic myeloid leukaemia:0.53), (VILAZODONE HYDROCHLORIDE:Chronic myeloid leukaemia:0.59), (VINORELBINE TARTRATE:Chronic myeloid leukaemia:0.26), (VINORELBINE\VINORELBINE TARTRATE:Chronic myeloid leukaemia:0.28), (YEAST:Chronic myeloid leukaemia:0.27), (ZOLMITRIPTAN:Chronic myeloid leukaemia:0.6), (BUMETANIDE:Chronic myelomonocytic leukaemia:0.21), (ENALAPRIL:Chronic myelomonocytic leukaemia:0.31), (PANTOPRAZOLE:Chronic myelomonocytic leukaemia:0.16), (SACUBITRIL\VALSARTAN:Chronic myelomonocytic leukaemia:0.2), (TORSEMIDE:Chronic myelomonocytic leukaemia:1), (.ALPHA.-TOCOPHEROL\VITAMINS:Chronic obstructive pulmonary disease:0.64), (ABACAVIR SULFATE\DOLUTEGRAVIR SODIUM\LAMIVUDINE:Chronic obstructive pulmonary disease:1.64), (ABIRATERONE:Chronic obstructive pulmonary disease:0.19), (ABOBOTULINUMTOXINA:Chronic obstructive pulmonary disease:0.96), (ACAMPROSATE:Chronic obstructive pulmonary disease:0.3), (ACECLOFENAC:Chronic obstructive pulmonary disease:0.86), (ACETAMINOPHEN\CAFFEINE\CARISOPRODOL\DICLOFENAC SODIUM:Chronic obstructive pulmonary disease:0.26), (ACETAMINOPHEN\CAFFEINE\OPIUM:Chronic obstructive pulmonary disease:1.33), (ACETAMINOPHEN\DICHLORALPHENAZONE\ISOMETHEPTENE:Chronic obstructive pulmonary disease:0.64), (AGALSIDASE BETA:Chronic obstructive pulmonary disease:3.05), (ALBIGLUTIDE:Chronic obstructive pulmonary disease:1.52), (ALBUTEROL\GUAIFENESIN:Chronic obstructive pulmonary disease:0.88), (ALDESLEUKIN:Chronic obstructive pulmonary disease:0.51), (ALEMTUZUMAB:Chronic obstructive pulmonary disease:1.47), (ALGLUCOSIDASE ALFA:Chronic obstructive pulmonary disease:0.89), (ALISKIREN HEMIFUMARATE\HYDROCHLOROTHIAZIDE:Chronic obstructive pulmonary disease:0.39), (ALISKIREN\HYDROCHLOROTHIAZIDE:Chronic obstructive pulmonary disease:0.11), (ALOGLIPTIN:Chronic obstructive pulmonary disease:0.46), (AMOXAPINE:Chronic obstructive pulmonary disease:0.59), (AMPICILLIN SODIUM:Chronic obstructive pulmonary disease:0.6), (ANAGLIPTIN:Chronic obstructive pulmonary disease:0.27), (ANAGRELIDE:Chronic obstructive pulmonary disease:0.47), (ANAKINRA:Chronic obstructive pulmonary disease:0.38), (ANTIHISTAMINES NOS:Chronic obstructive pulmonary disease:1.62), (ARCTOSTAPHYLOS UVA-URSI LEAF\URTICA DIOICA:Chronic obstructive pulmonary disease:0.07), (ARIPIPRAZOLE LAUROXIL:Chronic obstructive pulmonary disease:0.87), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Chronic obstructive pulmonary disease:0.79), (ASIAN GINSENG:Chronic obstructive pulmonary disease:1.14), (ATORVASTATIN CALCIUM\EZETIMIBE:Chronic obstructive pulmonary disease:0.65), (AVOCADO OIL\SOYBEAN OIL:Chronic obstructive pulmonary disease:0.6), (AZITHROMYCIN MONOHYDRATE:Chronic obstructive pulmonary disease:0.35), (BELATACEPT:Chronic obstructive pulmonary disease:0.36), (BENIDIPINE:Chronic obstructive pulmonary disease:0.29), (BETAMETHASONE DIPROPIONATE\CALCIPOTRIENE HYDRATE:Chronic obstructive pulmonary disease:0.07), (BETAMETHASONE DIPROPIONATE\CALCIPOTRIENE\CALCIPOTRIENE HYDRATE:Chronic obstructive pulmonary disease:2.12), (BIFIDOBACTERIUM LONGUM\LACTOBACILLUS ACIDOPHILUS\LACTOBACILLUS RHAMNOSUS:Chronic obstructive pulmonary disease:0.36), (BORON:Chronic obstructive pulmonary disease:0.06), (CABOZANTINIB:Chronic obstructive pulmonary disease:0.26), (CAFFEINE\DIPYRONE\ORPHENADRINE:Chronic obstructive pulmonary disease:0.62), (CAJUPUT OIL\EUCALYPTUS OIL\PEPPERMINT OIL:Chronic obstructive pulmonary disease:0.27), (CALCIUM BICARBONATE:Chronic obstructive pulmonary disease:0.45), (CALCIUM CARBONATE\ERGOCALCIFEROL:Chronic obstructive pulmonary disease:0.43), (CALCIUM CARBONATE\ETIDRONATE DISODIUM:Chronic obstructive pulmonary disease:0.61), (CALCIUM CARBONATE\MAGNESIUM HYDROXIDE:Chronic obstructive pulmonary disease:0.13), (CALCIUM PANTOTHENATE\CYANOCOBALAMIN\NIACINAMIDE\PYRIDOXINE HYDROCHLORIDE\RIBOFLAVIN\THIAMINE HYDROCHLORIDE:Chronic obstructive pulmonary disease:0.06), (CAMPHOR\DIPHENHYDRAMINE\ZINC OXIDE:Chronic obstructive pulmonary disease:0.49), (CANAGLIFLOZIN\METFORMIN HYDROCHLORIDE:Chronic obstructive pulmonary disease:2.48), (CARPERITIDE:Chronic obstructive pulmonary disease:0.01), (CARTEOLOL:Chronic obstructive pulmonary disease:0.7), (CEFOTAXIME\CEFOTAXIME SODIUM:Chronic obstructive pulmonary disease:0.59), (CEFPROZIL:Chronic obstructive pulmonary disease:0.59), (CERIVASTATIN SODIUM:Chronic obstructive pulmonary disease:0.83), (CHLORAL HYDRATE:Chronic obstructive pulmonary disease:0), (CHLORDIAZEPOXIDE\CLIDINIUM:Chronic obstructive pulmonary disease:0.12), (CHLORPROMAZINE HYDROCHLORIDE:Chronic obstructive pulmonary disease:0.05), (CHLORPROTHIXENE:Chronic obstructive pulmonary disease:0.13), (CHOLINE\TRAZODONE HYDROCHLORIDE:Chronic obstructive pulmonary disease:0.25), (CICLOPIROX OLAMINE:Chronic obstructive pulmonary disease:0.07), (CIFENLINE:Chronic obstructive pulmonary disease:0.56), (CINACALCET:Chronic obstructive pulmonary disease:0.08), (CISAPRIDE:Chronic obstructive pulmonary disease:0.21), (CITICOLINE:Chronic obstructive pulmonary disease:0.13), (CLENBUTEROL HYDROCHLORIDE:Chronic obstructive pulmonary disease:0.35), (CLODRONATE DISODIUM:Chronic obstructive pulmonary disease:0.19), (CLOXACILLIN SODIUM:Chronic obstructive pulmonary disease:0.6), (CODEINE PHOSPHATE\DICLOFENAC SODIUM:Chronic obstructive pulmonary disease:0.01), (COSYNTROPIN:Chronic obstructive pulmonary disease:0.04), (CRANBERRY CONCENTRATE:Chronic obstructive pulmonary disease:0.12), (CYPROTERONE:Chronic obstructive pulmonary disease:0.35), (DARUNAVIR\RITONAVIR:Chronic obstructive pulmonary disease:0.29), (DEFERIPRONE:Chronic obstructive pulmonary disease:0.32), (DEOXYRIBONUCLIEC ACID:Chronic obstructive pulmonary disease:0.11), (DEXMETHYLPHENIDATE HYDROCHLORIDE:Chronic obstructive pulmonary disease:0.1), (DEXTRAN:Chronic obstructive pulmonary disease:0.08), (DIACEREIN:Chronic obstructive pulmonary disease:0.06), (DIAZOXIDE:Chronic obstructive pulmonary disease:0.23), (DICLOFENAC DIETHYLAMINE:Chronic obstructive pulmonary disease:0.17), (DILAZEP:Chronic obstructive pulmonary disease:0.3), (DIPHENIDOL HYDROCHLORIDE:Chronic obstructive pulmonary disease:0.38), (DIPYRONE\PROMETHAZINE HYDROCHLORIDE:Chronic obstructive pulmonary disease:1.44), (DISOPYRAMIDE:Chronic obstructive pulmonary disease:0.84), (DOBUTAMINE HYDROCHLORIDE:Chronic obstructive pulmonary disease:1.21), (DOCUSATE SODIUM\SENNOSIDE A\SENNOSIDE B:Chronic obstructive pulmonary disease:0.04), (DOLASETRON MESYLATE:Chronic obstructive pulmonary disease:1.56), (DOXYLAMINE SUCCINATE\PYRIDOXINE HYDROCHLORIDE:Chronic obstructive pulmonary disease:0.05), (DROPERIDOL:Chronic obstructive pulmonary disease:0.1), (DROSPIRENONE\ETHINYL ESTRADIOL:Chronic obstructive pulmonary disease:0.29), (ECALLANTIDE:Chronic obstructive pulmonary disease:0.12), (EDARAVONE:Chronic obstructive pulmonary disease:0.62), (EMTRICITABINE:Chronic obstructive pulmonary disease:0.58), (EMTRICITABINE\RILPIVIRINE HYDROCHLORIDE\TENOFOVIR DISOPROXIL FUMARATE:Chronic obstructive pulmonary disease:1.02), (EMTRICITABINE\RILPIVIRINE\TENOFOVIR DISOPROXIL FUMARATE:Chronic obstructive pulmonary disease:0.39), (EPIRUBICIN HYDROCHLORIDE:Chronic obstructive pulmonary disease:0.06), (ERGOLOID MESYLATES:Chronic obstructive pulmonary disease:0.31), (ERIBULIN:Chronic obstructive pulmonary disease:0.71), (ERIBULIN MESYLATE:Chronic obstructive pulmonary disease:0.53), (ERYTHROMYCIN\SULFISOXAZOLE:Chronic obstructive pulmonary disease:0.38), (ESMOLOL HYDROCHLORIDE:Chronic obstructive pulmonary disease:0.35), (ESTRADIOL BENZOATE:Chronic obstructive pulmonary disease:0.01), (ESTROGENS\METHYLTESTOSTERONE:Chronic obstructive pulmonary disease:0.02), (ETHINYL ESTRADIOL\NORGESTREL:Chronic obstructive pulmonary disease:0.5), (ETOPOSIDE PHOSPHATE:Chronic obstructive pulmonary disease:0.26), (ETRETINATE:Chronic obstructive pulmonary disease:1.65), (FELODIPINE\METOPROLOL SUCCINATE:Chronic obstructive pulmonary disease:0.23), (FERRIC HYDROXIDE:Chronic obstructive pulmonary disease:0.59), (FERRIC OXYHYDROXIDE:Chronic obstructive pulmonary disease:0.01), (FERRIC POLYSACCHARIDE COMPLEX:Chronic obstructive pulmonary disease:0.17), (FERROUS BISGLYCINATE:Chronic obstructive pulmonary disease:1), (FESOTERODINE:Chronic obstructive pulmonary disease:0.68), (FEXOFENADINE HYDROCHLORIDE\PSEUDOEPHEDRINE HYDROCHLORIDE:Chronic obstructive pulmonary disease:0.09), (FILGRASTIM-SNDZ:Chronic obstructive pulmonary disease:0.15), (FLAVIN ADENINE DINUCLEOTIDE:Chronic obstructive pulmonary disease:0.13), (FLUOCINOLONE:Chronic obstructive pulmonary disease:0.12), (FLUOXETINE HYDROCHLORIDE\OLANZAPINE:Chronic obstructive pulmonary disease:0.14), (FLUPENTIXOL:Chronic obstructive pulmonary disease:0.13), (FURSULTIAMINE:Chronic obstructive pulmonary disease:0.54), (FURSULTIAMINE HYDROCHLORIDE:Chronic obstructive pulmonary disease:0.07), (FUSIDATE SODIUM:Chronic obstructive pulmonary disease:0.06), (GABAPENTIN ENACARBIL:Chronic obstructive pulmonary disease:0.13), (GLIMEPIRIDE\ROSIGLITAZONE MALEATE:Chronic obstructive pulmonary disease:0.85), (GLUCONATE:Chronic obstructive pulmonary disease:0.22), (GOLD:Chronic obstructive pulmonary disease:0.09), (GUAIFENESIN\PHENYLEPHRINE HYDROCHLORIDE:Chronic obstructive pulmonary disease:0.17), (HALCINONIDE:Chronic obstructive pulmonary disease:0.31), (HORSE CHESTNUT:Chronic obstructive pulmonary disease:0.51), (HYDROCORTISONE ACETATE:Chronic obstructive pulmonary disease:1.8), (IBRITUMOMAB TIUXETAN:Chronic obstructive pulmonary disease:0.5), (ICOSAPENT:Chronic obstructive pulmonary disease:0.43), (INOTUZUMAB OZOGAMICIN:Chronic obstructive pulmonary disease:0.45), (INSULIN ASPART\INSULIN DEGLUDEC:Chronic obstructive pulmonary disease:0.52), (INTERFERON:Chronic obstructive pulmonary disease:2.01), (INTERFERON ALFA-2B:Chronic obstructive pulmonary disease:1.46), (INTERFERON GAMMA-1B:Chronic obstructive pulmonary disease:1.2), (IRON POLYMALTOSE:Chronic obstructive pulmonary disease:0.59), (ISONIAZID\PYRAZINAMIDE\RIFAMPIN:Chronic obstructive pulmonary disease:0.32), (ISONIAZID\RIFAMPIN:Chronic obstructive pulmonary disease:0.4), (ITOPRIDE:Chronic obstructive pulmonary disease:0.21), (LANREOTIDE:Chronic obstructive pulmonary disease:1.24), (LAPATINIB DITOSYLATE:Chronic obstructive pulmonary disease:1.48), (LENVATINIB:Chronic obstructive pulmonary disease:1.14), (LEVOBUNOLOL:Chronic obstructive pulmonary disease:0.08), (LEVOSIMENDAN:Chronic obstructive pulmonary disease:0.47), (LOMUSTINE:Chronic obstructive pulmonary disease:0.24), (LOXOPROFEN SODIUM DIHYDRATE:Chronic obstructive pulmonary disease:0.04), (LUMACAFTOR:Chronic obstructive pulmonary disease:0.77), (LUTETIUM LU-177:Chronic obstructive pulmonary disease:1.03), (LYCOPENE:Chronic obstructive pulmonary disease:0.09), (MAGNESIUM PIDOLATE:Chronic obstructive pulmonary disease:0.39), (MAGNESIUM\PYRIDOXINE:Chronic obstructive pulmonary disease:0.09), (MAPROTILINE HYDROCHLORIDE:Chronic obstructive pulmonary disease:0.31), (MECLIZINE MONOHYDROCHLORIDE\NIACIN:Chronic obstructive pulmonary disease:0.05), (MECLOZINE DIHYDROCHLORIDE:Chronic obstructive pulmonary disease:0.11), (METFORMIN HYDROCHLORIDE\ROSIGLITAZONE MALEATE:Chronic obstructive pulmonary disease:1.03), (METHENAMINE MANDELATE:Chronic obstructive pulmonary disease:0.71), (METRELEPTIN:Chronic obstructive pulmonary disease:0.93), (METYRAPONE:Chronic obstructive pulmonary disease:0.01), (MIGLUSTAT:Chronic obstructive pulmonary disease:0.55), (MILNACIPRAN:Chronic obstructive pulmonary disease:0.24), (MILRINONE:Chronic obstructive pulmonary disease:0.6), (MILRINONE LACTATE:Chronic obstructive pulmonary disease:0.25), (MITIGLINIDE CALCIUM HYDRATE\VOGLIBOSE:Chronic obstructive pulmonary disease:0.44), (MITOXANTRONE HYDROCHLORIDE:Chronic obstructive pulmonary disease:0.18), (MORNIFLUMATE:Chronic obstructive pulmonary disease:0.28), (NALTREXONE HYDROCHLORIDE:Chronic obstructive pulmonary disease:0.21), (NEOMYCIN:Chronic obstructive pulmonary disease:0.9), (NICARDIPINE:Chronic obstructive pulmonary disease:2.37), (OCRELIZUMAB:Chronic obstructive pulmonary disease:0.14), (OCTREOTIDE:Chronic obstructive pulmonary disease:1.92), (OMEGA-3-CARBOXYLIC ACIDS:Chronic obstructive pulmonary disease:0.04), (OSPEMIFENE:Chronic obstructive pulmonary disease:0.62), (PANTETHINE:Chronic obstructive pulmonary disease:0.29), (PAPAYA:Chronic obstructive pulmonary disease:0.19), (PEGZEREPOETIN ALFA:Chronic obstructive pulmonary disease:0.58), (PENICILLAMINE:Chronic obstructive pulmonary disease:0.48), (PENICILLIN V BENZATHINE:Chronic obstructive pulmonary disease:0.28), (PENTAMIDINE ISETHIONATE:Chronic obstructive pulmonary disease:0.92), (PENTOSTATIN:Chronic obstructive pulmonary disease:0.97), (PERMETHRIN:Chronic obstructive pulmonary disease:0.1), (PINAVERIUM BROMIDE:Chronic obstructive pulmonary disease:0.24), (PIRENZEPINE:Chronic obstructive pulmonary disease:0), (PITOFENONE:Chronic obstructive pulmonary disease:0.24), (PORFIMER SODIUM:Chronic obstructive pulmonary disease:0.61), (POTASSIUM CITRATE\TRISODIUM CITRATE DIHYDRATE:Chronic obstructive pulmonary disease:0.6), (POTASSIUM PHOSPHATE, MONOBASIC:Chronic obstructive pulmonary disease:0.61), (PRAMOXINE HYDROCHLORIDE:Chronic obstructive pulmonary disease:0.53), (PREGNENOLONE:Chronic obstructive pulmonary disease:0.22), (PROBUCOL:Chronic obstructive pulmonary disease:0.67), (PROPIVERINE:Chronic obstructive pulmonary disease:0.19), (PROTRIPTYLINE HYDROCHLORIDE:Chronic obstructive pulmonary disease:1.44), (PSORALEN:Chronic obstructive pulmonary disease:0.19), (PYRIDOXAL:Chronic obstructive pulmonary disease:0.3), (QUAZEPAM:Chronic obstructive pulmonary disease:0.04), (QUINACRINE:Chronic obstructive pulmonary disease:1.19), (QUINAGOLIDE HYDROCHLORIDE:Chronic obstructive pulmonary disease:0.08), (RETINOL:Chronic obstructive pulmonary disease:1.41), (RIFABUTIN:Chronic obstructive pulmonary disease:0.51), (RILPIVIRINE:Chronic obstructive pulmonary disease:0.01), (ROSIGLITAZONE:Chronic obstructive pulmonary disease:0.37), (RUCAPARIB CAMSYLATE:Chronic obstructive pulmonary disease:0.29), (RUPATADINE FUMARATE:Chronic obstructive pulmonary disease:0.36), (SCOPOLAMINE HYDROBROMIDE:Chronic obstructive pulmonary disease:0.18), (SELEGILINE HYDROCHLORIDE:Chronic obstructive pulmonary disease:1.22), (SERRAPEPTASE:Chronic obstructive pulmonary disease:0.75), (SIBUTRAMINE HYDROCHLORIDE:Chronic obstructive pulmonary disease:0.97), (SILICON DIOXIDE:Chronic obstructive pulmonary disease:0.55), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Chronic obstructive pulmonary disease:0.51), (SOMATOSTATIN:Chronic obstructive pulmonary disease:0), (SORBITOL:Chronic obstructive pulmonary disease:1.09), (SPIRAMYCIN:Chronic obstructive pulmonary disease:0.09), (SPIRULINA:Chronic obstructive pulmonary disease:0.03), (ST. JOHN'S WORT:Chronic obstructive pulmonary disease:0.24), (SUFENTANIL CITRATE:Chronic obstructive pulmonary disease:0.55), (TBO-FILGRASTIM:Chronic obstructive pulmonary disease:0.02), (TEDUGLUTIDE:Chronic obstructive pulmonary disease:0.39), (TEDUGLUTIDE\WATER:Chronic obstructive pulmonary disease:1.09), (TEGASEROD MALEATE:Chronic obstructive pulmonary disease:0.06), (TELOTRISTAT ETHYL:Chronic obstructive pulmonary disease:0.84), (TEMSIROLIMUS:Chronic obstructive pulmonary disease:0.82), (TETRABENAZINE:Chronic obstructive pulmonary disease:0.56), (TIANEPTINE SODIUM:Chronic obstructive pulmonary disease:0.09), (TIMOLOL\TRAVOPROST:Chronic obstructive pulmonary disease:0.93), (TOFACITINIB:Chronic obstructive pulmonary disease:0.12), (TRIMEPRAZINE:Chronic obstructive pulmonary disease:0.31), (VALDECOXIB:Chronic obstructive pulmonary disease:0.99), (VANDETANIB:Chronic obstructive pulmonary disease:0.3), (VELAGLUCERASE ALFA:Chronic obstructive pulmonary disease:0.45), (VINORELBINE\VINORELBINE TARTRATE:Chronic obstructive pulmonary disease:0.55), (VINPOCETINE:Chronic obstructive pulmonary disease:0.4), (ZALTOPROFEN:Chronic obstructive pulmonary disease:0.09), (ZIV-AFLIBERCEPT:Chronic obstructive pulmonary disease:0.55), (ZOFENOPRIL:Chronic obstructive pulmonary disease:0.03), (SELEXIPAG:Chronic respiratory failure:0.22), (WARFARIN SODIUM:Chronic respiratory failure:0.06), (.ALPHA.1-PROTEINASE INHIBITOR HUMAN:Chronic sinusitis:0.72), (ANASTROZOLE:Chronic sinusitis:0.61), (BISACODYL OR DOCUSATE SODIUM:Chronic sinusitis:0.29), (CANAKINUMAB:Chronic sinusitis:0.09), (CARISOPRODOL:Chronic sinusitis:0.09), (CERTOLIZUMAB PEGOL:Chronic sinusitis:0.95), (CLOTRIMAZOLE:Chronic sinusitis:0.15), (COLCHICINE:Chronic sinusitis:0.28), (DACLIZUMAB:Chronic sinusitis:0.38), (DALFAMPRIDINE:Chronic sinusitis:0.53), (DASATINIB:Chronic sinusitis:0.27), (DILTIAZEM HYDROCHLORIDE:Chronic sinusitis:0.69), (ENALAPRIL MALEATE:Chronic sinusitis:0.07), (ENOXAPARIN:Chronic sinusitis:0.48), (ESOMEPRAZOLE:Chronic sinusitis:0.06), (ESZOPICLONE:Chronic sinusitis:0.56), (ETORICOXIB:Chronic sinusitis:0.03), (EVOLOCUMAB:Chronic sinusitis:0.73), (FERROUS SULFATE:Chronic sinusitis:0.46), (FERROUS SULFATE\FERROUS SULFATE, DRIED:Chronic sinusitis:0.79), (FORMOTEROL FUMARATE\MOMETASONE FUROATE:Chronic sinusitis:0.7), (GLIPIZIDE:Chronic sinusitis:0.28), (GLYCOPYRRONIUM:Chronic sinusitis:0.2), (HYDROMORPHONE:Chronic sinusitis:0.36), (ILOPROST:Chronic sinusitis:0), (INDOMETHACIN:Chronic sinusitis:0.13), (INFLUENZA VIRUS VACCINE:Chronic sinusitis:0.07), (LUMACAFTOR:Chronic sinusitis:0.22), (NEBIVOLOL:Chronic sinusitis:0.05), (NILOTINIB:Chronic sinusitis:0.2), (OLANZAPINE:Chronic sinusitis:0.07), (OXCARBAZEPINE:Chronic sinusitis:0.42), (OXYBUTYNIN CHLORIDE:Chronic sinusitis:0.03), (OXYGEN:Chronic sinusitis:0.61), (PANTOPRAZOLE MAGNESIUM:Chronic sinusitis:0.69), (PAROXETINE:Chronic sinusitis:0.18), (POLYETHYLENE GLYCOLS:Chronic sinusitis:0.59), (PRAMIPEXOLE DIHYDROCHLORIDE:Chronic sinusitis:0.44), (RANOLAZINE:Chronic sinusitis:0.43), (ROFLUMILAST:Chronic sinusitis:0.25), (SECUKINUMAB:Chronic sinusitis:0.62), (TELMISARTAN:Chronic sinusitis:0.38), (TERIFLUNOMIDE:Chronic sinusitis:0.56), (UMECLIDINIUM BROMIDE\VILANTEROL TRIFENATATE:Chronic sinusitis:0.7), (VANCOMYCIN HYDROCHLORIDE:Chronic sinusitis:0.07), (ZINC:Chronic sinusitis:0.04), (BISOPROLOL FUMARATE:Chronic spontaneous urticaria:0.09), (CALCIUM:Chronic spontaneous urticaria:0.91), (CERTOLIZUMAB PEGOL:Chronic spontaneous urticaria:0.75), (DICLOFENAC SODIUM:Chronic spontaneous urticaria:0.1), (EVOLOCUMAB:Chronic spontaneous urticaria:0.35), (FERROUS SULFATE\FERROUS SULFATE, DRIED:Chronic spontaneous urticaria:0.01), (IRON:Chronic spontaneous urticaria:0.03), (LOVASTATIN:Chronic spontaneous urticaria:0.19), (MELOXICAM:Chronic spontaneous urticaria:0.27), (METRONIDAZOLE:Chronic spontaneous urticaria:0.42), (POTASSIUM:Chronic spontaneous urticaria:0.09), (SECUKINUMAB:Chronic spontaneous urticaria:0.32), (TOCILIZUMAB:Chronic spontaneous urticaria:0.01), (PYRAZINAMIDE:Chylothorax:0.56), (ACETAMINOPHEN\OXYCODONE HYDROCHLORIDE\OXYCODONE TEREPHTHALATE:Circadian rhythm sleep disorder:0.1), (ALBIGLUTIDE:Circadian rhythm sleep disorder:0.07), (AMIODARONE:Circadian rhythm sleep disorder:0.49), (BUDESONIDE:Circadian rhythm sleep disorder:1.26), (CALCIUM CARBONATE\CHOLECALCIFEROL:Circadian rhythm sleep disorder:0.33), (CANDESARTAN CILEXETIL:Circadian rhythm sleep disorder:0.39), (CEPHALEXIN:Circadian rhythm sleep disorder:0.43), (CERTOLIZUMAB PEGOL:Circadian rhythm sleep disorder:0.18), (CETIRIZINE HYDROCHLORIDE\PSEUDOEPHEDRINE HYDROCHLORIDE:Circadian rhythm sleep disorder:0.07), (CIPROFLOXACIN HYDROCHLORIDE:Circadian rhythm sleep disorder:0.47), (CLINDAMYCIN\CLINDAMYCIN PHOSPHATE:Circadian rhythm sleep disorder:0.31), (CLOBAZAM:Circadian rhythm sleep disorder:0.02), (CORTISONE\HYDROCORTISONE:Circadian rhythm sleep disorder:0.04), (DACLIZUMAB:Circadian rhythm sleep disorder:0.5), (DAPAGLIFLOZIN PROPANEDIOL:Circadian rhythm sleep disorder:0.22), (DEVICE:Circadian rhythm sleep disorder:0.35), (DILTIAZEM:Circadian rhythm sleep disorder:0.29), (DOXAZOSIN MESYLATE:Circadian rhythm sleep disorder:0.05), (DOXAZOSIN\DOXAZOSIN MESYLATE:Circadian rhythm sleep disorder:0.17), (DUTASTERIDE:Circadian rhythm sleep disorder:0.51), (ELETRIPTAN HYDROBROMIDE:Circadian rhythm sleep disorder:0.84), (ENOXAPARIN:Circadian rhythm sleep disorder:0.73), (ESTROGENS, CONJUGATED\MEDROXYPROGESTERONE ACETATE:Circadian rhythm sleep disorder:0.34), (EVEROLIMUS:Circadian rhythm sleep disorder:0.09), (EVOLOCUMAB:Circadian rhythm sleep disorder:0.87), (FESOTERODINE FUMARATE:Circadian rhythm sleep disorder:0.36), (FISH OIL\OMEGA-3 FATTY ACIDS:Circadian rhythm sleep disorder:0.02), (FLUTICASONE:Circadian rhythm sleep disorder:0.22), (GLUCOSAMINE:Circadian rhythm sleep disorder:0.04), (HEPARIN SODIUM:Circadian rhythm sleep disorder:0.01), (HYDROXYZINE PAMOATE:Circadian rhythm sleep disorder:1.25), (HYOSCYAMINE:Circadian rhythm sleep disorder:0.74), (IBRUTINIB:Circadian rhythm sleep disorder:0.25), (IPRATROPIUM:Circadian rhythm sleep disorder:0.43), (IRBESARTAN:Circadian rhythm sleep disorder:0.35), (METHENAMINE HIPPURATE:Circadian rhythm sleep disorder:0.1), (METHYLPREDNISOLONE ACETATE:Circadian rhythm sleep disorder:0.22), (METHYLPREDNISOLONE SODIUM SUCCINATE:Circadian rhythm sleep disorder:0.3), (METRONIDAZOLE:Circadian rhythm sleep disorder:0.56), (METRONIDAZOLE\METRONIDAZOLE HYDROCHLORIDE:Circadian rhythm sleep disorder:0.05), (MOXIFLOXACIN HYDROCHLORIDE:Circadian rhythm sleep disorder:0.63), (NITROFURANTOIN:Circadian rhythm sleep disorder:0.26), (OXYGEN:Circadian rhythm sleep disorder:0.52), (PANTOPRAZOLE MAGNESIUM:Circadian rhythm sleep disorder:0.26), (PAROXETINE:Circadian rhythm sleep disorder:0.38), (PENICILLIN:Circadian rhythm sleep disorder:0.38), (PHENOBARBITAL:Circadian rhythm sleep disorder:0.97), (PYRIDOXINE HYDROCHLORIDE:Circadian rhythm sleep disorder:0.14), (RABEPRAZOLE:Circadian rhythm sleep disorder:0.03), (RANOLAZINE:Circadian rhythm sleep disorder:1.27), (TERIFLUNOMIDE:Circadian rhythm sleep disorder:0.98), (TIMOLOL:Circadian rhythm sleep disorder:0.87), (TOLTERODINE TARTRATE:Circadian rhythm sleep disorder:0.27), (UMECLIDINIUM BROMIDE\VILANTEROL TRIFENATATE:Circadian rhythm sleep disorder:0.87), (ZINC:Circadian rhythm sleep disorder:0.4), (AMIODARONE HYDROCHLORIDE:Clear cell renal cell carcinoma:0.17), (DIGOXIN:Clear cell renal cell carcinoma:0.53), (DILTIAZEM HYDROCHLORIDE:Clear cell renal cell carcinoma:0.13), (IBRUTINIB:Clear cell renal cell carcinoma:0.08), (LOSARTAN:Clear cell renal cell carcinoma:0.27), (LOVASTATIN:Clear cell renal cell carcinoma:0.24), (NAPROXEN:Clear cell renal cell carcinoma:0.12), (VALSARTAN:Clear cell renal cell carcinoma:0.29), (ANASTROZOLE:Clostridium difficile colitis:0.33), (AZITHROMYCIN ANHYDROUS:Clostridium difficile colitis:0.27), (BUMETANIDE:Clostridium difficile colitis:0.38), (CALCITRIOL:Clostridium difficile colitis:0.07), (CANDESARTAN CILEXETIL:Clostridium difficile colitis:0.43), (CASPOFUNGIN:Clostridium difficile colitis:0.17), (CERTOLIZUMAB PEGOL:Clostridium difficile colitis:0.35), (CLONIDINE:Clostridium difficile colitis:0.16), (DEXLANSOPRAZOLE:Clostridium difficile colitis:0.42), (DICLOFENAC:Clostridium difficile colitis:0.36), (DILTIAZEM HYDROCHLORIDE:Clostridium difficile colitis:0.35), (DIVALPROEX SODIUM:Clostridium difficile colitis:0.34), (DOXYCYCLINE:Clostridium difficile colitis:0.03), (EPINEPHRINE:Clostridium difficile colitis:0.4), (ERGOCALCIFEROL:Clostridium difficile colitis:0.3), (EVOLOCUMAB:Clostridium difficile colitis:0.11), (GLIMEPIRIDE:Clostridium difficile colitis:0.2), (ILOPROST:Clostridium difficile colitis:0.26), (LERCANIDIPINE:Clostridium difficile colitis:0.08), (LOVASTATIN:Clostridium difficile colitis:0.51), (MELOXICAM:Clostridium difficile colitis:0.34), (METHYLPREDNISOLONE SODIUM SUCCINATE:Clostridium difficile colitis:0.75), (NIFEDIPINE:Clostridium difficile colitis:0.09), (NILOTINIB:Clostridium difficile colitis:0.18), (OXCARBAZEPINE:Clostridium difficile colitis:0.15), (PRAVASTATIN:Clostridium difficile colitis:0.12), (SACUBITRIL\VALSARTAN:Clostridium difficile colitis:0.19), (SECUKINUMAB:Clostridium difficile colitis:0.05), (THALIDOMIDE:Clostridium difficile colitis:0.33), (.ALPHA.1-PROTEINASE INHIBITOR HUMAN:Clostridium difficile infection:0.09), (ACETAMINOPHEN\BUTALBITAL\CAFFEINE:Clostridium difficile infection:0.11), (AMPHETAMINE ASPARTATE\AMPHETAMINE SULFATE\DEXTROAMPHETAMINE SACCHARATE\DEXTROAMPHETAMINE SULFATE:Clostridium difficile infection:0.19), (AZELASTINE:Clostridium difficile infection:0.07), (BECLOMETHASONE DIPROPIONATE:Clostridium difficile infection:0.26), (BENAZEPRIL\BENAZEPRIL HYDROCHLORIDE:Clostridium difficile infection:0.17), (BUDESONIDE\FORMOTEROL FUMARATE:Clostridium difficile infection:0.16), (CANAKINUMAB:Clostridium difficile infection:0.08), (CANDESARTAN CILEXETIL\HYDROCHLOROTHIAZIDE:Clostridium difficile infection:0.52), (CARVEDILOL PHOSPHATE:Clostridium difficile infection:0.47), (CHLORHEXIDINE:Clostridium difficile infection:0.06), (CLOBAZAM:Clostridium difficile infection:0.01), (CYCLOBENZAPRINE:Clostridium difficile infection:0.51), (DESLORATADINE:Clostridium difficile infection:0.19), (DOFETILIDE:Clostridium difficile infection:0.04), (DOXAZOSIN MESYLATE:Clostridium difficile infection:0.07), (ELETRIPTAN HYDROBROMIDE:Clostridium difficile infection:0.2), (ELOTUZUMAB:Clostridium difficile infection:0.08), (ENALAPRIL MALEATE:Clostridium difficile infection:0.5), (EVEROLIMUS:Clostridium difficile infection:0.51), (EVOLOCUMAB:Clostridium difficile infection:0.41), (EZETIMIBE:Clostridium difficile infection:0.61), (EZETIMIBE\SIMVASTATIN:Clostridium difficile infection:0.07), (FLECAINIDE:Clostridium difficile infection:0.41), (FORMOTEROL FUMARATE\MOMETASONE FUROATE:Clostridium difficile infection:0.14), (GLYCOPYRRONIUM\INDACATEROL:Clostridium difficile infection:0.23), (HYDROCHLOROTHIAZIDE\LOSARTAN POTASSIUM:Clostridium difficile infection:0.08), (ILOPROST:Clostridium difficile infection:0.47), (INDAPAMIDE\PERINDOPRIL:Clostridium difficile infection:0.17), (INTERFERON GAMMA-1B:Clostridium difficile infection:0.02), (ITRACONAZOLE:Clostridium difficile infection:0.08), (LACOSAMIDE:Clostridium difficile infection:0.19), (LATANOPROST:Clostridium difficile infection:0.22), (LITHIUM CARBONATE:Clostridium difficile infection:0.3), (MESNA:Clostridium difficile infection:0.11), (METAXALONE:Clostridium difficile infection:0.31), (METHADONE HYDROCHLORIDE:Clostridium difficile infection:0.11), (METHENAMINE HIPPURATE:Clostridium difficile infection:0.16), (METHYLPREDNISOLONE ACETATE:Clostridium difficile infection:0.56), (NEBIVOLOL:Clostridium difficile infection:0.88), (NIACIN:Clostridium difficile infection:0.07), (NILOTINIB:Clostridium difficile infection:0.91), (OLODATEROL HYDROCHLORIDE\TIOTROPIUM BROMIDE MONOHYDRATE:Clostridium difficile infection:0.16), (PANTOPRAZOLE MAGNESIUM:Clostridium difficile infection:0.76), (PAZOPANIB HYDROCHLORIDE:Clostridium difficile infection:0), (PEMETREXED DISODIUM:Clostridium difficile infection:0.01), (PENICILLIN:Clostridium difficile infection:0.16), (PERINDOPRIL:Clostridium difficile infection:0.05), (PRAMIPEXOLE DIHYDROCHLORIDE:Clostridium difficile infection:0.06), (PYRIDOXINE HYDROCHLORIDE:Clostridium difficile infection:0.24), (RALOXIFENE HYDROCHLORIDE:Clostridium difficile infection:0.01), (ROFLUMILAST:Clostridium difficile infection:0.16), (SACUBITRIL\VALSARTAN:Clostridium difficile infection:0.19), (SECUKINUMAB:Clostridium difficile infection:0.11), (SELEXIPAG:Clostridium difficile infection:0.08), (SODIUM OXYBATE:Clostridium difficile infection:0.14), (SOTALOL:Clostridium difficile infection:0.46), (SUMATRIPTAN\SUMATRIPTAN SUCCINATE:Clostridium difficile infection:0.02), (TELMISARTAN:Clostridium difficile infection:0.37), (TOCILIZUMAB:Clostridium difficile infection:0.18), (UMECLIDINIUM BROMIDE\VILANTEROL TRIFENATATE:Clostridium difficile infection:0.39), (VARENICLINE TARTRATE:Clostridium difficile infection:0), (VITAMIN B:Clostridium difficile infection:0.43), (ZONISAMIDE:Clostridium difficile infection:0.14), (AMPHETAMINE ASPARTATE\AMPHETAMINE SULFATE\DEXTROAMPHETAMINE SACCHARATE\DEXTROAMPHETAMINE SULFATE:Cluster headache:0.07), (CERTOLIZUMAB PEGOL:Cluster headache:0.17), (OXCARBAZEPINE:Cluster headache:0.12), (PAROXETINE:Cluster headache:0), (TERIFLUNOMIDE:Cluster headache:0.3), (ACARBOSE:Coagulopathy:0.62), (ACETAMINOPHEN\OXYCODONE\OXYC ODONE HYDROCHLORIDE:Coagulopathy:0.55), (ADO-TRASTUZUMAB EMTANSINE:Coagulopathy:0.42), (AGALSIDASE BETA:Coagulopathy:0.61), (ALBIGLUTIDE:Coagulopathy:0.15), (ALCOHOL:Coagulopathy:0.45), (ALEMTUZUMAB:Coagulopathy:0.22), (ALISKIREN HEMIFUMARATE:Coagulopathy:0.09), (AMANTADINE:Coagulopathy:0.57), (AMINO ACIDS\DEXTROSE\ELECTROLYTES NOS\SOYBEAN OIL\VITAMINS:Coagulopathy:0.18), (AMISULPRIDE:Coagulopathy:0.56), (AMPICILLIN:Coagulopathy: 0.5), (APREPITANT:Coagulopathy:0.36), (AZELASTINE:Coagulopathy:0.1), (AZITH ROMYCIN DIHYDRATE:Coagulopathy:0.58), (BICALUTAMIDE:Coagulopathy:0.06), (BRINZOLAMIDE:Coagulopathy:0.4), (CALCIUM CITRATE:Coagulopathy:0.93), (CALCIUM\\MAGNESIUM:Coagulopathy:0.06), (CALCIUM\M AGNESIUM\ZINC:Coagulopathy:0.19), (CANAGLIFLOZIN\METFORMIN HYDROCHLORIDE:Coagulopathy:0.61), (CANAKINUMAB:Coagulopathy:0.26), (CANDESA RTAN CILEXETIL\HYDROCHLOROTHIAZIDE:Coagulopathy:1.43), (CARVEDILOL PHOSPHATE:Coagulopathy:1.41), (CEFOTAXIME SODIUM:Coagulopathy:0.67), (CEFUROXIME:Coagulopathy:0.22), (CETIRIZINE HYDROCHLORIDE\PSEUDOEPHEDRINE HYDROCHLORIDE:Coagulopathy:0.02), (CHONDROITIN SULFATE (BOVINE)\GLUCOSAMINE HYDROCHLORIDE:Coagulopathy:0.09), (CICLESONIDE:Coagulopathy:0.39), (CLINDAMYC IN:Coagulopathy:0.96), (COD LIVER OIL:Coagulopathy:0.17), (CORTICOTROPIN:Coagulopathy:0.28), (CRIZOTINIB:Coagulopathy: 0.25), (DACLIZUMAB:Coagulopathy:0.53), (DANAZOL:Coagulopathy:0.68), (DEFLAZACOR T:Coagulopathy:0.4), (DIGITOXIN:Coagulopathy:0.61), (DIPYRONE:Coagulopathy:0.43), (DO XEPIN HYDROCHLORIDE:Coagulopathy:0.33), (DULAGLUTIDE:Coagulopathy:0.2), (DUTASTERI DE\TAMSULOSIN HYDROCHLORIDE:Coagulopathy:0.04), (ECONAZOLE:Coagulopathy:0.18), (EMPAGLIFLO ZIN\LINAGLIPTIN:Coagulopathy:0.75), (EPIRUBICIN:Coagulopathy:0.31), (ETIMICIN SULFATE:Coagulopathy:0.21), (ETORICOXIB:Coagulopathy:0.56), (FENOFIBRIC ACID:Coagulopathy:0.08), (FENOTEROL HYDROBROMIDE\IPRATROPIUM BROMIDE:Coagulopathy:0.08), (FENTANYL CITRATE:Coagulopathy:0.15), (FERROUS SULFATE\GLYCINE:Coagulopathy:0.13), (FINGOLIMOD HYDROCHLORIDE:Coagulopathy:0.19), (FISH OIL\OMEGA-3 FATTY ACIDS:Coagulopathy:0.07), (FLUTICASONE FUROATE:Coagulopathy:0.5), (FLUVOXAMINE MALEATE:Coagulopathy:0.08), (FORMOTEROL:Coagulopathy:0.27), (FORMOTEROL FUMARATE:Coagulopathy:0.68), (FORMOTEROL FUMARATE DIHYDRATE\MOMETASONE FUROATE:Coagulopathy:0.64), (FORMOTEROL FUMARATE\MOMETASONE FUROATE:Coagulopathy:0.1), (FUSIDIC ACID:Coagulopathy:0.08), (GLYCOPYRRONIUM\lNDACATEROL:Coagulopathy:0.89), (HER BALS\TURMERIC:Coagulopathy:0.3), (HYDROCHLOROTHIAZIDE\TELMISARTAN:Coagulopathy: 0.56), (ILOPROST:Coagulopathy:0.89), (IMIPRAMINE HYDROCHLORIDE:Coagulopathy:0.73), (INDAPAMIDE\PERINDOPRIL:Coagulopathy:1.03), (INSULIN DEGLUDEC:Coagulopathy:0.21), (INTERFERON ALFA-2A: Coagulopathy:0.08), (IRINOTECAN:Coagulopathy:0.17), (IRINOTECAN HYDROCHLORIDE:Coagulopathy:0.3), (ISOSORBIDE DINITRATE:Coagulopathy:0.19), (IXAZOMIB:Coagulopathy:0.23), (LACTOBACILLUS RHAMNOSUS:Coagulopathy:0.12), (LERCANIDIPINE HYDROCHLORIDE:Coagulopathy:0.89), (LEUCOGEN:Coagulopathy:0.16), (LEUCOVORIN CALCIUM:Coagulopathy:0.47), (LEVOLEUCOVORIN CALCIUM:Coagulopathy:0.07), (LOMITAPIDE MESYLATE:Coagulopathy:0.31), (METHENAMINE HIPPURATE:Coagulopathy:0.78), (METHENOLONE ENANTHATE:Coagulopathy:0.15), (METHIMAZOLE:Coagulopathy:0.27), (METHYLPREDNI SOLONE SODIUM SUCCINATE:Coagulopathy:1.45), (MUPIROCIN\MUPIROCIN CALCIUM:Coagulopathy:0.31), (MYCOPHENOLATE MOFETIL\MYCOPHENOLATE MOFETIL HYDROCHLORIDE:Coagulopathy:0.52), (NATEGLINIDE:Coagulopathy:0.46), (NICOTINE:Coagulopathy: 0.11), (NITROFURANTOIN\NITROFURANTOIN MONOHYDRATE:Coagulopathy:0.15), (NOREPINEPHRINE BITARTRATE:Coagulopathy:0.24), (OSELTAMIVIR:Coagulopathy:0.07), (PANCRELIPASE:C oagulopathy:0.04), (PARICALCITOL:Coagulopathy:0.13), (PATIROMER:Coagulopathy:0.23), (PAZOPANIB HYDROCHLORIDE:Coagulopathy:0.75), (PEGFILGRASTIM:Coagulopathy:0.57), (PEGINTE RFERON BETA-1A:Coagulopathy:0.62), (PEMETREXED:Coagulopathy:0.17), (PEMETREXED DISODIUM:Coagulopathy:0.47), (PENTOSAN POLYSULFATE SODIUM:Coagulopathy:0.02), (PHOSPHOCREATINE SODIUM:Coagulopathy:0.08), (PREDNISOLONE ACETATE:Coagulopathy:0.34), (RALOXIFENE HYDROCHLORIDE:Coagulopathy:0.52), (REGORAFENIB:Coagulopathy:0.23), (REPAGLINI DE:Coagulopathy:0.03), (RIZATRIPTAN BENZOATE:Coagulopathy:0.62), (ROFLUMILAST:Coagulopathy:0.95), (SECUKINUMAB:Coagulopathy: 0.61), (SELEXIPAG:Coagulopathy:0.26), (SODIUM PHOSPHATE, DIBASIC\SODIUM PHOSPHATE, MONOBASIC:Coagulopathy:0.04), (SOTALOL HYDROCHLORIDE:Coagulopathy:0.08), (SUMATRIPTAN\SUMATRIPTAN SUCCINATE:Coagulopathy:1.03), (TERBUTALINE:Coagulopathy:0.08), (TETRACYCLINE:C oagulopathy:0.18), (THALIDOMIDE:Coagulopathy:1.09), (THIAMINE:Coagulopathy:0.06), (TI MOLOL:Coagulopathy:0.91), (TIOTROPIUM:Coagulopathy:0.81), (TRIAMTERENE:Coagulopathy: 0.54), (UMECLIDINIUM BROMIDE:Coagulopathy:0.17), (UMECLIDINIUM BROMIDE\VILANTEROL TRIFENATATE:Coagulopathy:0.96), (VALGANCICLOVIR HYDROCHLORIDE:Coagulopathy:0.98), (VINCRISTINE SULFATE:Coagulopathy:0.57), (VITAMIN B:Coagulopathy:1.04), (ZINC:Coagulopathy:0.39), (ZONISAMIDE:Coagulopathy:0.33), (UNSPE CIFIED INGREDIENT:Coeliac disease:0.06), (ALCOHOL:Cognitive disorder:0.28), (ANASTROZOLE:Cognitive disorder:0.15), (BUMETANIDE:Cognitive disorder:0.22), (CIPROFLOXACIN HYDROCHLORIDE:Cognitive disorder:0.04), (CLINDAMYCIN\CLINDAMYCIN PHOSPHATE:Cognitive disorder:0.1), (COLCHICINE:Cognitive disorder:0.14), (DEVICE:Cognitive disorder:0.05), (DICLOFENAC:Cognitive disorder:0.3), (DILTIAZEM:Cognitive disorder:0.53), (DOXAZOSIN\DOXAZOSIN MESYLATE:Cognitive disorder:0.09), (ENOXAPARIN:Cognitive disorder:0.37), (EVOLOCUMAB:Cognitive disorder:0.63), (FLECAINIDE:Cognitive disorder:0.18), (HYDROMORPHONE HYDROCHLORIDE:Cognitive disorder:0.03), (HYDROXYZINE PAMOATE:Cognitive disorder:0.04), (IBRUTINIB:Cognitive disorder:1.03), (INDAPAMIDE:Cognitive disorder:0.26), (INDAPAMIDE\PERINDOPRIL:Cognitive disorder:0.27), (LITHIUM CARBONATE:Cognitive disorder:0.75), (METAXALONE:Cognitive disorder:0.15), (MORPHINE:Cognitive disorder:0.59), (NILOTINIB:Cognitive disorder:0), (OXYGEN:Cognitive disorder:0.06), (PHENYTOIN:Cognitive disorder:0.02), (PROMETHAZINE\PROMETHAZINE HYDROCHLORIDE:Cognitive disorder:0.11), (RABEPRAZOLE SODIUM:Cognitive disorder:0.23), (RANOLAZINE:Cognitive disorder:0.5), (SACUBITRIL\VALSARTAN:Cognitive disorder:0.76), (SOTALOL:Cognitive disorder:0.36), (TEMAZEPAM:Cognitive disorder:0.37), (TRAMADOL HYDROCHLORIDE:Cognitive disorder:0.03), (VARENICLINE TARTRATE:Cognitive disorder:0.88), (VITAMIN B:Cognitive disorder:0.26), (ACETAMINOPHEN\OXYCODONE HYDROCHLORIDE\OXYCODONE TEREPHTHALATE:Colitis:0.04), (ACETAMINOPHEN\OXYCODONE\OXYCODONE HYDROCHLORIDE:Colitis:0.22), (ACETAMINOPHEN\PROPOXYPHENE HYDROCHLORIDE:Colitis:0.09), (AGALSIDASE BETA:Colitis:0.06), (ALCOHOL:Colitis:0.05), (ALFACALCIDOL:Colitis:0.23), (ALISKIREN HEMIFUMARATE:Colitis:0), (AMANTADINE:Colitis:0.11), (AMIKACIN:Colitis:0.5), (AMIN O ACIDS\DEXTROSE\ELECTROLYTES NOS:Colitis:0.08), (AMLODIPINE BESYLATE\HYDROCHLOROTHIAZIDE\OLMESARTAN MEDOXOMIL:Colitis:0.16),(ARMODAFINIL:Colitis:1.02), (ATOVAQUONE:Colitis:0.01), (A ZELASTINE:Colitis:0.04), (AZELASTINE HYDROCHLORIDE:Colitis:0.02), (BENAZEPRIL\BENAZEPRIL HYDROCHLORIDE:Colitis:0.36), (BETAMETHASONE SODIUM PHOSPHATE:Colitis:0.06), (BISACODYL:Colitis:0.49), (BRINZOLAMIDE:Colitis:0.3), (CALC ITONIN SALMON:Colitis:0.13), (CALCIUM ACETATE:Colitis:0.84), (CALCIUM CARBONATE\VITAMIN D:Colitis:0.43), (CALCIUM CITRATE:Colitis:0.82), (CANAGLIFLOZIN\METFORMIN HYDROCHLORIDE:Colitis:0.19), (CANAKINUMAB:Colitis:0.41), (CANDESARTAN CILEXETIL\HYDROCHLOROTHIAZIDE:Colitis:1.22), (CARFILZOMIB:Colitis:0.9), (CARV EDILOL PHOSPHATE:Colitis:0.72), (CHONDROITIN SULFATE (BOVINE)\GLUCOSAMINE HYDROCHLORIDE:Colitis:0.35), (CLOZAPINE:Colitis:0.81), (CORTICOTROPIN:Colitis:0.2), (CYPROHEPTADINE:Colitis:0.1), (DACLIZUMAB:Colitis:0.89), (DANAZOL:Colitis:0.17), (D ASATINIB:Colitis:1), (DOXEPIN HYDROCHLORIDE:Colitis:0.27), (DULAGLUTIDE:Colitis:0.19), (EDOXABAN TOSYLATE:Colitis:0.07), (ELETRIPTAN HYDROBROMIDE:Colitis:1.03), (EMPAGLIFLOZIN\LINAGLIPTIN:Colitis:0.3), (EPLEREN ONE:Colitis:0.12), (ESTROGENS, CONJUGATED\MEDROXYPROGESTERONE ACETATE:Colitis:0.31), (FENOFIBRIC ACID:Colitis:0.13), (FLUOCINONIDE:Colitis:0.16), (FLUTICASONE FUROATE:Colitis:0.39), (FORMOTEROL FUMARATE:Colitis:0.47), (FUSIDIC ACID:Colitis:0.41), (GLYCOPYRRONIUM\INDACATEROL:Colitis:0.5), (HEPARIN CALCIUM:Colitis:0.02), (HERBALS\TURMERIC:Colitis:0.21), (HYDROCHLOROTHIAZIDE \IRBESARTAN:Colitis:0.68), (HYDROXYZINE PAMOATE:Colitis:1.2), (IBRUTINIB:Colitis:1.04), (ILOPROST:Colitis:0.12), (INDAPAMIDE: Colitis:0.61), (INSULIN DEGLUDEC:Colitis:0.03), (INVESTIGATIONAL PRODUCT:Colitis:0.16), (LABETALOL HYDROCHLORIDE:Colitis:0.66), (LACTASE:Colitis:0.22), (LEDIPASVIR\SOFOSBUVIR:Colitis: 0.57), (LITHIUM CARBONATE:Colitis:0.99), (LOMITAPIDE MESYLATE:Colitis:0.02), (METHIMAZOLE:Colitis:0.67), (METOLAZONE:Colitis:0.08), (MI DODRINE:Colitis:0.11), (MINOCYCLINE\MINOCYCLINE HYDROCHLORIDE:Colitis:0.81), (MUPIROCIN\MUPIROCIN CALCIUM:Colitis:0.39), (NILOTINIB:Colitis:1.05), (NINTEDANIB:Colitis:0.25), (NITROFUR ANTOIN:Colitis:0.92), (NITROFURANTOIN\NITROFURANTOIN MONOHYDRATE:Colitis:0.21), (OLODATEROL HYDROCHLORIDE\TIOTROPIUM BROMIDE MONOHYDRATE:Colitis:0.32), (OSELTAMIVIR:Colitis:0.5), (OXYBUTYNIN CHLORIDE:Colitis:0.61), (PAZOPANIB HYDROCHLORIDE:Colitis:0.06), (PEMETREXED DISODIUM:Colitis:0.18), (PHENOBARBITAL:Colitis:0.86), (PHENYTOIN SODIUM:Colitis:0.2), (PONATINIB:Colitis:0.76), (PREDNISOLONE ACETATE:Colitis:0.24), (PREDNISOLONE\PREDNISOLONE ACETATE\PREDNISOLONE SODIUM SUCCINATE:Colitis:0.34), (ROFLUMILAST:Colitis:0.66), (ROPINIROLE HYDROCHLORIDE:Colitis:0.4), (SENNA LEAF\SENNOSIDES\SENNOSIDES A AND B:Colitis:0.34), (SULFAMETHOXAZOLE:Colitis:1.09), (TERBINAFINE HYDROCHLORIDE:Colitis:0.3), (TETRACYCLINE:Colitis:0.93), (THALIDOMIDE:Colitis:0.69), (TOCOPHEROL:Colitis:0.07), (TOLTERODINE TARTRATE:Colitis:0.54), (VILAZODONE HYDROCHLORIDE:Colitis:0.23), (ZINC:Colitis:0.37), (ZONISAMIDE:Colitis:0.02), (TERIFLU NOMIDE:Colitis microscopic:0.4), (TOPIRAMATE:Colitis microscopic:0.46), (VARENICLINE TARTRATE:Colitis microscopic:0.03), (.ALPHA.-TOCOPHEROL\VITAMINS:Colitis ulcerative:0), (.ALPHA.1-PROTEINASE INHIBITOR HUMAN:Colitis ulcerative:0.58), (ABIRATERONE ACETATE:Colitis ulcerative:0.03), (ACETAMINOPHEN\CAFFEINE\OPIUM:Colitis ulcerative:0.61), (ACETAMINOPHEN\PROPOXYPHENE NAPSYLATE:Colitis ulcerative:1.07), (ADO-TRASTUZUMAB EMTANSINE:Colitis ulcerative:0.36), (AGALSIDASE BETA:Colitis ulcerative:0.77), (ALBUTEROL\IPRATROPIUM:Colitis ulcerative:0.47), (ALEMTUZUMAB:Colitis ulcerative:0.51), (ALISKIREN:Colitis ulcerative:0.3), (AMBROXOL:Colitis ulcerative:0.11), (AMIKACIN SULFATE:Colitis ulcerative:0.04), (AMLODIPINE\PERINDOPRIL:Colitis ulcerative:0.02), (AMOXICILLIN\CLARITHROMYCIN\ESOMEPRAZOLE:Colitis ulcerative:0.48), (ANHYDROUS CITRIC ACID\ASPIRIN\SODIUM BICARBONATE:Colitis ulcerative:0), (ARIPIPRAZOLE LAUROXIL:Colitis ulcerative:0.27), (ASPIRIN\MAGNESIUM OXIDE:Colitis ulcerative:0.23), (AXITINIB:Colitis ulcerative:0.47), (AZILSARTAN KAMEDOXOMIL:Colitis ulcerative:1.11), (BECLOMETHASONE\FORMOTEROL:Colitis ulcerative:0.05), (BERAPROST SODIUM:Colitis ulcerative:0.05), (BETAMETHASONE DIPROPIONATE\CALCIPOTRIENE:Colitis ulcerative:0.12), (BETAMETHASONE DIPROPIONATE\CLOTRIMAZOLE:Colitis ulcerative:0.37), (BETAMETHASONE\CALCIPOTRIENE:Colitis ulcerative:1.24), (BETHANECHOL CHLORIDE:Colitis ulcerative:0.5), (BUPIVACAINE HYDROCHLORIDE:Colitis ulcerative:0.01), (CALCIUM CARBONATE\ERGOCALCIFEROL\RETINOL:Colitis ulcerative:0.47), (CALCIUM\MAGNESIUM\ZINC:Colitis ulcerative:0.77), (CANAGLIFLOZIN\METFORMIN HYDROCHLORIDE:Colitis ulcerative:1.15), (CANDESARTAN CILEXETIL\HYDROCHLOROTHIAZIDE:Colitis ulcerative:2.2), (CARBOMER:Colitis ulcerative:0.11), (CASANTHRANOL\DOCUSATE SODIUM:Colitis ulcerative:0.68), (CASPOFUNGIN:Colitis ulcerative:0.02), (CEFEPIME\CEFEPIME HYDROCHLORIDE:Colitis ulcerative:0.19), (CETUXIMAB:Colitis ulcerative:0.45), (CEVIMELINE HYDROCHLORIDE:Colitis ulcerative:0.58), (CHLORAMBUCIL:Colitis ulcerative:0.2), (CLIKSTAR:Colitis ulcerative:0.08), (CLINDAMYCIN PHOSPHATE:Colitis ulcerative:1.08), (COBICISTAT\ELVITEGRAVIR\EMTRICITABINE\TENOFOVIR DISOPROXIL FUMARATE:Colitis ulcerative:0.06), (CORTICOTROPIN:Colitis ulcerative:0.96), (DACLIZUMAB:Colitis ulcerative:1.63), (DANAZOL:Colitis ulcerative:0.56), (DANTROLENE SODIUM:Colitis ulcerative:0.12), (DAPAGLIFLOZIN PROPANEDIOL\METFORMIN HYDROCHLORIDE:Colitis ulcerative:0.06), (DEXAMETHASONE ACETATE:Colitis ulcerative:0.5), (DEXAMETHASONE SODIUM PHOSPHATE:Colitis ulcerative:0.38), (DIFLUNISAL:Colitis ulcerative:0.27), (DIPYRONE\PROMETHAZINE HYDROCHLORIDE:Colitis ulcerative:0.3), (DOLASETRON MESYLATE:Colitis ulcerative:0.11), (DOXEPIN HYDROCHLORIDE:Colitis ulcerative:1.37), (DOXYLAMINE SUCCINATE:Colitis ulcerative:0.47), (ECONAZOLE:Colitis ulcerative:1.27), (ELOTUZUMAB:Colitis ulcerative:0.5), (EMPAGLIFLOZIN\LINAGLIPTIN:Colitis ulcerative:0.87), (ESTRADIOL HEMIHYDRATE:Colitis ulcerative:0.02), (ETHACRYNIC ACID:Colitis ulcerative:0.19), (ETHINYL ESTRADIOL\ETHYNODIOL:Colitis ulcerative:0.5), (ETRETINATE:Colitis ulcerative:0.36), (FENOTEROL HYDROBROMIDE\IPRATROPIUM BROMIDE:Colitis ulcerative:0.47), (FLUOCINOLONE ACETONIDE:Colitis ulcerative:0.57), (FLURBIPROFEN:Colitis ulcerative:0.09), (FORMOTEROL:Colitis ulcerative:1.36), (FORMOTEROL FUMARATE DIHYDRATE:Colitis ulcerative:0.04), (FOSFOMYCIN:Colitis ulcerative:0.1), (FROVATRIPTAN SUCCINATE:Colitis ulcerative:0.02), (GLIPIZIDE\METFORMIN HYDROCHLORIDE:Colitis ulcerative:0.09), (GLYCOPYRROLATE\INDACATEROL MALEATE:Colitis ulcerative:0.39), (HEPARIN CALCIUM:Colitis ulcerative:0.46), (HYOSCYAMINE SULFATE\METHENAMINE\METHYLENE BLUE\PHENYL SALICYLATE\SODIUM PHOSPHATE, MONOBASIC, MONOHYDRATE:Colitis ulcerative:0.01), (IDELALISIB:Colitis ulcerative:0.32), (IMATINIB MESYLATE:Colitis ulcerative:1.32), (IMIGLUCERASE:Colitis ulcerative:0.44), (INSULIN BEEF:Colitis ulcerative:0.73), (INTERFERON ALFA-2A:Colitis ulcerative:0.32), (INTERFERON GAMMA-1B:Colitis ulcerative:0.11), (IRINOTECAN HYDROCHLORIDE:Colitis ulcerative:0.55), (ISOPROPYL ALCOHOL\LEUPROLIDE ACETATE\NORETHINDRONE ACETATE:Colitis ulcerative:0.75), (IXAZOMIB:Colitis ulcerative:0.32), (LAPATINIB DITOSYLATE:Colitis ulcerative:0.04), (LENOGRASTIM:Colitis ulcerative:0.04), (LEVOLEUCOVORIN CALCIUM:Colitis ulcerative:0.29), (LEVOMEFOLATE CALCIUM\METHYLCOBALAMIN\PYRIDOXAL PHOSPHATE ANHYDROUS:Colitis ulcerative:0.5), (LOMITAPIDE MESYLATE:Colitis ulcerative:0.89), (LUTETIUM LU-177:Colitis ulcerative:0.17), (MAGNESIUM ASPARTATE HYDROCHLORIDE:Colitis ulcerative:0.24), (MAGNESIUM GLUCONATE:Colitis ulcerative:0.44), (MEGESTROL:Colitis ulcerative:0.22), (METFORMIN HYDROCHLORIDE\VILDAGLIPTIN:Colitis ulcerative:0.52), (METHENAMINE HIPPURATE:Colitis ulcerative:1.54), (METHYLPREDNISOLONE ACETATE:Colitis ulcerative:2.26), (METRELEPTIN:Colitis ulcerative:0.05), (MEXILETINE:Colitis ulcerative:0.14), (MICONAZOLE NITRATE:Colitis ulcerative:0.7), (MOEXIPRIL HYDROCHLORIDE:Colitis ulcerative:0.35), (MORPHINE SULFATE\NALTREXONE HYDROCHLORIDE:Colitis ulcerative:0.34), (NALOXONE:Colitis ulcerative:0.3), (NEOMYCIN:Colitis ulcerative:0.43), (NEPAFENAC:Colitis ulcerative:0.21), (NICARDIPINE:Colitis ulcerative:0.9), (NICARDIPINE HYDROCHLORIDE:Colitis ulcerative:0.08), (NIRAPARIB:Colitis ulcerative:0.3), (NOREPINEPHRINE BITARTRATE:Colitis ulcerative:0.1), (OSELTAMIVIR:Colitis ulcerative:0.75), (OXAPROZIN:Colitis ulcerative:0.35), (OXYBUTYNIN\OXYBUTYNIN CHLORIDE:Colitis ulcerative:0.37), (OXYMETAZOLINE:Colitis ulcerative:0.42), (OXYMORPHONE:Colitis ulcerative:0.14), (PALIPERIDONE PALMITATE:Colitis ulcerative:0.61), (PATIROMER:Colitis ulcerative:0.33), (PEMETREXED:Colitis ulcerative:0.05), (PENICILLIN G:Colitis ulcerative:0.49), (PERAMPANEL:Colitis ulcerative:0.01), (PHENAZOPYRIDINE HYDROCHLORIDE:Colitis ulcerative:0.15), (PILOCARPINE HYDROCHLORIDE:Colitis ulcerative:0.21), (PNEUMOCOCCAL VACCINE, POLYVALENT 23:Colitis ulcerative:0.4), (POLYETHYLENE GLYCOL 4000:Colitis ulcerative:0.21), (POMALIDOMIDE:Colitis ulcerative:0.17), (PONATINIB:Colitis ulcerative:1.31), (PROPAFENONE HYDROCHLORIDE:Colitis ulcerative:0.46), (PROTRIPTYLINE HYDROCHLORIDE:Colitis ulcerative:0.47), (QUINACRINE:Colitis ulcerative:0.45), (REMIFENTANIL:Colitis ulcerative:0.38), (RETINOL:Colitis ulcerative:1.32), (RIFABUTIN:Colitis ulcerative:0.06), (RILPIVIRINE HYDROCHLORIDE:Colitis ulcerative:0.42), (ROFECOXIB:Colitis ulcerative:0.01), (ROFLUMILAST:Colitis ulcerative:1.69), (SALMETEROL:Colitis ulcerative:0.72), (SARPOGRELATE:Colitis ulcerative:0.28), (SELEGILINE:Colitis ulcerative:0.23), (SENNA LEAF:Colitis ulcerative:0.07), (SEVELAMER CARBONATE:Colitis ulcerative:0.35), (SEVELAMER HYDROCHLORIDE:Colitis ulcerative:0.4), (SIBUTRAMINE HYDROCHLORIDE:Colitis ulcerative:0.04), (SODIUM POLYSTYRENE SULFONATE:Colitis ulcerative:0.16), (SULFACETAMIDE SODIUM\SULFUR:Colitis ulcerative:0.01), (TASIMELTEON:Colitis ulcerative:0.06), (TEDUGLUTIDE:Colitis ulcerative:0.55), (TELOTRISTAT ETHYL:Colitis ulcerative:0.09), (TERBUTALINE:Colitis ulcerative:0.97), (THEOPHYLLINE:Colitis ulcerative:0.31), (UMECLIDINIUM BROMIDE:Colitis ulcerative:0.54), (UMECLIDINIUM BROMIDE\VILANTEROL TRIFENATATE:Colitis ulcerative:1.97), (URAPIDIL:Colitis ulcerative:0.28), (VOGLIBOSE:Colitis ulcerative:0.11), (YEAST:Colitis ulcerative:0.38), (ZICONOTIDE ACETATE:Colitis ulcerative:0.03), (ZILEUTON:Colitis ulcerative:0.42), (ABIRATERONE:Colon cancer:0.08), (ACETAMINOPHEN\OXYCODONE HYDROCHLORIDE\OXYCODONE TEREPHTHALATE:Colon cancer:0.31), (ACETAZOLAMIDE:Colon cancer:1.18), (ACETYLCYSTEINE:Colon cancer:0.19), (ACTIVATED CHARCOAL:Colon cancer:0.22), (AGALSIDASE BETA:Colon cancer:0.27), (ALDESLEUKIN:Colon cancer:0.24), (ALEMTUZUMAB:Colon cancer:0.2), (ALISKIREN:Colon cancer:0.02), (ALISKIREN HEMIFUMARATE:Colon cancer:0.2), (AMLODIPINE BESYLATE\HYDROCHLOROTHIAZIDE\VALSARTAN:Colon cancer:0.12), (AMLODIPINE\VALSARTAN:Colon cancer:0.28), (ANTIHISTAMINES NOS:Colon cancer:0.01), (AZELASTINE:Colon cancer:0.13), (AZELASTINE HYDROCHLORIDE:Colon cancer:0.01), (AZOSEMIDE:Colon cancer:0.7), (BENAZEPRIL\BENAZEPRIL HYDROCHLORIDE:Colon cancer:0.59), (BEPRIDIL:Colon cancer:0.19), (BERAPROST SODIUM:Colon cancer:0.02), (BUPRENORPHINE HYDROCHLORIDE\NALOXONE HYDROCHLORIDE:Colon cancer:0.24), (CALCITONIN SALMON:Colon cancer:1.08), (CALCIUM CARBONATE\VITAMIN D:Colon cancer:0.41), (CALCIUM LACTATE:Colon cancer:0.26), (CANAGLIFLOZIN\METFORMIN HYDROCHLORIDE:Colon cancer:0.58), (CARBOXYMETHYLCELLULOSE SODIUM:Colon cancer:0.21), (CARFILZOMIB:Colon cancer:1.02), (CARVEDILOL PHOSPHATE:Colon cancer:1), (CASANTHRANOL\DOCUSATE SODIUM:Colon cancer:0.29), (CEFAZOLIN:Colon cancer:0.21), (CEFUROXIME:Colon cancer:0.06), (CERTOLIZUMAB PEGOL:Colon cancer:0.7), (CHLORDIAZEPOXIDE HYDROCHLORIDE\CLIDINIUM BROMIDE:Colon cancer:0.15), (CILASTATIN SODIUM\IMIPENEM:Colon cancer:0.04), (CLOBAZAM:Colon cancer:0.38), (CLOZAPINE:Colon cancer:0.87), (CORTICOTROPIN:Colon cancer:0.04), (CORTISONE\HYDROCORTISONE:Colon cancer:0.68), (DABRAFENIB MESYLATE:Colon cancer:0.59), (DACLIZUMAB:Colon cancer:0.29), (DANAZOL:Colon cancer:0.45), (DEFLAZACORT:Colon cancer:0.48), (DEVICE:Colon cancer:0.33), (DIGITOXIN:Colon cancer:0.32), (DOFETILIDE:Colon cancer:0.55), (DOPAMINE HYDROCHLORIDE:Colon cancer:0.07), (DOXEPIN HYDROCHLORIDE:Colon cancer:0.19), (DURVALUMAB:Colon cancer:0.24), (DUTASTERIDE\TAMSULOSIN HYDROCHLORIDE:Colon cancer:0.09), (ELDECALCITOL:Colon cancer:0.2), (ELETRIPTAN HYDROBROMIDE:Colon cancer:0.86), (EMPAGLIFLOZIN\LINAGLIPTIN:Colon cancer:0.65), (ERIBULIN:Colon cancer:0.56), (ERIBULIN MESYLATE:Colon cancer:0.17), (ESOMEPRAZOLE MAGNESIUM\NAPROXEN:Colon cancer:0.15), (ETRETINATE:Colon cancer:0.31), (EVOLOCUMAB:Colon cancer:0.21), (FESOTERODINE FUMARATE:Colon cancer:0.96), (FISH OIL\OMEGA-3 FATTY ACIDS:Colon cancer:0.07), (FLECAINIDE:Colon cancer:1.27), (FLUDROCORTISONE ACETATE:Colon cancer:0.78), (FLUTICASONE FUROATE:Colon cancer:0.58), (FORMOTEROL:Colon cancer:0.07), (FUSIDIC ACID:Colon cancer:0.93), (GEFITINIB:Colon cancer:0.71), (GLYCOPYRRONIUM\INDACATEROL:Colon cancer:0.28), (HYDROCODONE BITARTRATE\IBUPROFEN:Colon cancer:0.15), (HYDROXYUREA:Colon cancer:0.65), (IDELALISIB:Colon cancer:0.07), (IMIPRAMINE HYDROCHLORIDE:Colon cancer:0.43), (INDAPAMIDE\PERINDOPRIL:Colon cancer:1.32), (INFLUENZA VIRUS VACCINE:Colon cancer:0.28), (IPRATROPIUM BROMIDE:Colon cancer:0.5), (IXAZOMIB:Colon cancer:0.42), (LACTASE:Colon cancer:0.25), (LACTOBACILLUS RHAMNOSUS:Colon cancer:0.37), (LANREOTIDE:Colon cancer:0.29), (LIOTHYRONINE:Colon cancer:0.03), (LISDEXAMFETAMINE DIMESYLATE:Colon cancer:0.69), (LITHIUM CARBONATE:Colon cancer:1.15), (LOMITAPIDE MESYLATE:Colon cancer:0.25), (MECLIZINE\MECLIZINE HYDROCHLORIDE:Colon cancer:0.85), (MERCAPTOPURINE:Colon cancer:0.21), (METAXALONE:Colon cancer:1.15), (METHENOLONE ENANTHATE:Colon cancer:0.16), (METHYLPREDNISOLONE ACETATE:Colon cancer:0.72), (MORPHINE SULFATE\NALTREXONE HYDROCHLORIDE:Colon cancer:0.25), (MYCOPHENOLATE MOFETIL\MYCOPHENOLATE MOFETIL HYDROCHLORIDE:Colon cancer:0.19), (NABUMETONE:Colon cancer:0.37), (NADROPARIN CALCIUM:Colon cancer:0.78), (OSIMERTINIB:Colon cancer:0.56), (PANTOPRAZOLE MAGNESIUM:Colon cancer:1.21), (PATIROMER:Colon cancer:0.22), (PEGINTERFERON BETA-1A:Colon cancer:0.15), (PEMETREXED:Colon cancer:1.23), (PEMETREXED DISODIUM:Colon cancer:0.78), (PIROXICAM:Colon cancer:0.05), (POLYETHYLENE GLYCOL 4000:Colon cancer:0.19), (POMALIDOMIDE:Colon cancer:0.04), (PROGESTERONE:Colon cancer:0.06), (RALOXIFENE HYDROCHLORIDE:Colon cancer:0.39), (RIVASTIGMINE:Colon cancer:0.16), (RIVASTIGMINE TARTRATE:Colon cancer:0.15), (RIZATRIPTAN BENZOATE:Colon cancer:0.76), (RUCAPARIB CAMSYLATE:Colon cancer:0.13), (SACUBITRIL\VALSARTAN:Colon cancer:0.38), (SIPULEUCEL-T:Colon cancer:0.11), (SULFAMETHOXAZOLE:Colon cancer:0.82), (SUMATRIPTAN\SUMATRIPTAN SUCCINATE:Colon cancer:0.67), (TAMOXIFEN CITRATE:Colon cancer:0.38), (TEDUGLUTIDE\WATER:Colon cancer:0.5), (TEMOZOLOMIDE:Colon cancer:0.52), (TERBINAFINE HYDROCHLORIDE:Colon cancer:0.61), (TERBUTALINE SULFATE:Colon cancer:0.25), (TERIFLUNOMIDE:Colon cancer:0.51), (TIOTROPIUM:Colon cancer:0.52), (UMECLIDINIUM BROMIDE:Colon cancer:0.23), (VANDETANIB:Colon cancer:0.1), (VILAZODONE HYDROCHLORIDE:Colon cancer:0.07), (VINORELBINE TARTRATE:Colon cancer:0.8), (VINORELBINE\VINORELBINE TARTRATE:Colon cancer:0.24), (ANASTROZOLE:Colon cancer metastatic:0.21), (BUDESONIDE:Colon cancer metastatic:0.58), (CALCITRIOL:Colon cancer metastatic:0.01), (CALCIUM CARBONATE\CHOLECALCIFEROL:Colon cancer metastatic:0.68), (CANDESARTAN CILEXETIL:Colon cancer metastatic:0.05), (CERTOLIZUMAB PEGOL:Colon cancer metastatic:0.43), (CLINDAMYCIN\CLINDAMYCIN PHOSPHATE:Colon cancer metastatic:0.11), (CLONIDINE:Colon cancer metastatic:0.05), (DABRAFENIB:Colon cancer metastatic:0.16), (DEVICE:Colon cancer metastatic:0.14), (DOCETAXEL:Colon cancer metastatic:0.05), (ENALAPRIL MALEATE:Colon cancer metastatic:0.01), (EPINEPHRINE:Colon cancer metastatic:0.04), (ESOMEPRAZOLE:Colon cancer metastatic:0.14), (EVOLOCUMAB:Colon cancer metastatic:0.48), (EZETIMIBE:Colon cancer metastatic:0.02), (FERROUS SULFATE\FERROUS SULFATE, DRIED:Colon cancer metastatic:0.63), (FLUCONAZOLE:Colon cancer metastatic:0.24), (IBRUTINIB:Colon cancer metastatic:0.44), (INDAPAMIDE\PERINDOPRIL:Colon cancer metastatic:0.18), (LINAGLIPTIN:Colon cancer metastatic:0.32), (METRONIDAZOLE:Colon cancer metastatic:0.17), (MONTELUKAST SODIUM:Colon cancer metastatic:0.22), (NAPROXEN:Colon cancer metastatic:0.67), (NILOTINIB:Colon cancer metastatic:0.53), (RANOLAZINE:Colon cancer metastatic:0.02), (SOTALOL:Colon cancer metastatic:0.23), (TERIFLUNOMIDE:Colon cancer metastatic:0.06), (TOPIRAMATE:Colon cancer metastatic:0.05), (ACARBOSE:Colonoscopy:0.02),(ARMODAFINIL:Colonoscopy:0.48), (CAR VEDILOL PHOSPHATE:Colonoscopy:0.27), (CERTOLIZUMAB PEGOL:Colonoscopy:0.96), (CLINDAMYCIN\CLINDAMYCIN PHOSPHATE:Colonoscopy:0.6), (CLOTRIMAZOLE:Colonoscopy:0.2), (CODEINE:Colonoscopy: 0.09), (DAPAGLIFLOZIN PROPANEDIOL:Colonoscopy:0.04), (DASATINIB:Colonoscopy:0.58), (DIVALPROEX SODIUM:Colonoscopy:0.9), (ELETRIPTAN HYDROBROMIDE:Colonoscopy:0.26), (ESZOPICLONE:Colonoscopy:0.57), (EVEROLIMUS: Colonoscopy:0.34), (EVOLOCUMAB:Colonoscopy:0.56), (FESOTERODINE FUMARATE:Colonoscopy:0.17), (FLECAINIDE:Colonoscopy:0.21), (FLUCONAZOLE:Colonoscopy: 0.26), (FLUTICASONE:Colonoscopy:0.28), (HYOSCYAMINE:Colonoscopy:0.42), (INSU LIN HUMAN:Colonoscopy:0.67), (IPRATROPIUM:Colonoscopy:0.22), (LERCANIDIPINE HYDROCHLORIDE:Colonoscopy:0.12), (METHYLPREDNISOLONE ACETATE:Colonoscopy:0.03), (MOXIFLOXACIN HYDROCHLORIDE:Colonoscopy:0.18), (NALOXONE HYDROCHLORIDE\OXYCODONE HYDROCHLORIDE:Colonoscopy:0.3), (NEBIVOLOL:Colonoscopy:0.21), (PANTOPRAZOLE MAGNESIUM:Colonoscopy:0.28), (PEGFILGRASTIM:Colonoscopy:0.03), (PENICILLIN:Colonoscopy: 0.14), (PHENOBARBITAL:Colonoscopy:0.23), (PYRIDOXINE:Colonoscopy:0.18), (R ABEPRAZOLE:Colonoscopy:0.09), (RIZATRIPTAN BENZOATE:Colonoscopy:0.36), (SACUBITRIL\VALSARTAN:Colonoscopy:0.62), (SAXAGLI PTIN HYDROCHLORIDE:Colonoscopy:0.02), (SECUKINUMAB:Colonoscopy:0.02), (SUMATRIPT AN:Colonoscopy:0.03), (TERIFLUNOMIDE:Colonoscopy:0.36), (TETRACYCLINE:Colonoscopy: 0.75), (TOCILIZUMAB:Colonoscopy:0.13), (TOPIRAMATE:Colonoscopy:1.82), (TORSEMI DE:Colonoscopy:0.55), (UMECLIDINIUM BROMIDE\VILANTEROL TRIFENATATE:Colonoscopy:0.36), (VARENICLINE TARTRATE:Colonoscopy:1.05), (VITAMIN B:Colonoscopy:0.76), (IRINOTECAN:Colony stimulating factor prophylaxis:0.28), (THALIDOMIDE:Colony stimulating factor prophylaxis:0.13), (VINCRISTINE SULFATE:Colony stimulating factor therapy:0.07), (ACARBOSE:Colorectal cancer:0.03), (ADO-TRASTUZUMAB EMTANSINE:Colorectal cancer:1.09), (ALENDRONIC ACID:Colorectal cancer:0.05), (ANASTROZOLE:Colorectal cancer:1.59), (ASPIRIN LYSINE:Colorectal cancer:0.42), (BENAZEPRIL HYDROCHLORIDE:Colorectal cancer:0.17), (BISACODYL OR DOCUSATE SODIUM:Colorectal cancer:0.38), (BUDESONIDE:Colorectal cancer:1.26), (BUMETANIDE:Colorectal cancer:0.73), (CALCITONIN SALMON:Colorectal cancer:0.04), (CERTOLIZUMAB PEGOL:Colorectal cancer:1.14), (CLOTRIMAZOLE:Colorectal cancer:0.42), (CLOZAPINE:Colorectal cancer:0.07), (DASATINIB:Colorectal cancer:0.92), (DESLORATADINE:Colorectal cancer:0.18), (DEVICE:Colorectal cancer:0.16), (DEXLANSOPRAZOLE:Colorectal cancer:1.24), (DIGOXIN:Colorectal cancer:1.01), (DOXAZOSIN MESYLATE:Colorectal cancer:0.25), (DOXAZOSIN\DOXAZOSIN MESYLATE:Colorectal cancer:0.1), (ENALAPRIL MALEATE:Colorectal cancer:0.69), (EPIRUBICIN:Colorectal cancer:0.78), (ERIBULIN:Colorectal cancer:0.44), (EVOLOCUMAB:Colorectal cancer:0.31), (EZETIMIBE:Colorectal cancer:0.55), (GEFITINIB:Colorectal cancer:0.22), (HEPARIN CALCIUM:Colorectal cancer:0.03), (IBRUTINIB:Colorectal cancer:1.06), (INDAPAMIDE\PERINDOPRIL:Colorectal cancer:0.84), (IPRATROPIUM:Colorectal cancer:0.04), (LABETALOL\LABETALOL HYDROCHLORIDE:Colorectal cancer:0.09), (LAPATINIB DITOSYLATE:Colorectal cancer:0.44), (LEDIPASVIR\SOFOSBUVIR:Colorectal cancer:0.75), (LERCANIDIPINE HYDROCHLORIDE:Colorectal cancer:0.47), (LINAGLIPTIN:Colorectal cancer:0.85), (LOVASTATIN:Colorectal cancer:1.32), (MECLIZINE\MECLIZINE HYDROCHLORIDE:Colorectal cancer:0.03), (METHYLPREDNISOLONE ACETATE:Colorectal cancer:0.29), (MOXIFLOXACIN HYDROCHLORIDE:Colorectal cancer:0.58), (NILOTINIB:Colorectal cancer:1.21), (OMEGA-3-ACID ETHYL ESTERS:Colorectal cancer:0.28), (OSIMERTINIB:Colorectal cancer:0.13), (OXYGEN:Colorectal cancer:0.29), (PAMIDRONATE DISODIUM:Colorectal cancer:0.27), (PANTOPRAZOLE MAGNESIUM:Colorectal cancer:0.48), (PEMETREXED DISODIUM:Colorectal cancer:0.17), (PERTUZUMAB:Colorectal cancer:0.34), (PRAMIPEXOLE DIHYDROCHLORIDE:Colorectal cancer:0.08), (PREDNISOLONE\PREDNISOLONE ACETATE\PREDNISOLONE SODIUM SUCCINATE:Colorectal cancer:0.02), (PRIMIDONE:Colorectal cancer:0.59), (RADIATION THERAPY:Colorectal cancer:0.12), (RANOLAZINE:Colorectal cancer:0.26), (ROFLUMILAST:Colorectal cancer:0.22), (SECUKINUMAB:Colorectal cancer:0.05), (SODIUM OXYBATE:Colorectal cancer:0.12), (SULFAMETHOXAZOLE:Colorectal cancer:0), (SUNITINIB MALATE:Colorectal cancer:0.21), (TEMOZOLOMIDE:Colorectal cancer:0.24), (VARENICLINE TARTRATE:Colorectal cancer:0.05), (VITAMIN B:Colorectal cancer:0.09), (ACARBOSE:Colorectal cancer metastatic:0.16), (AMILORIDE HYDROCHLORIDE\HYDROCHLOROTHIAZIDE:Colorectal cancer metastatic:0.07), (AMPHETAMINE ASPARTATE\AMPHETAMINE SULFATE\DEXTROAMPHETAMINE SACCHARATE\DEXTROAMPHETAMINE SULFATE:Colorectal cancer metastatic:0.04), (BENAZEPRIL HYDROCHLORIDE:Colorectal cancer metastatic:0.19), (CALCITONIN SALMON:Colorectal cancer metastatic:0.5), (CALCITRIOL:Colorectal cancer metastatic:0.92), (CALCIUM ACETATE:Colorectal cancer metastatic:0.07), (CALCIUM CITRATE:Colorectal cancer metastatic:0), (CARFILZOMIB:Colorectal cancer metastatic:0.06), (CEFOTAXIME SODIUM:Colorectal cancer metastatic:0.26), (CERTOLIZUMAB PEGOL:Colorectal cancer metastatic:0.99), (CILASTATIN SODIUM\IMIPENEM:Colorectal cancer metastatic:0.13), (CLOZAPINE:Colorectal cancer metastatic:0.44), (DABIGATRAN:Colorectal cancer metastatic:0.08), (DABRAFENIB MESYLATE:Colorectal cancer metastatic:0.06), (DIPYRONE:Colorectal cancer metastatic:0.11), (DIVALPROEX SODIUM:Colorectal cancer metastatic:0.41), (ECONAZOLE:Colorectal cancer metastatic:0.45), (EDOXABAN TOSYLATE:Colorectal cancer metastatic:0.4), (EPIRUBICIN:Colorectal cancer metastatic:0.43), (EPLERENONE:Colorectal cancer metastatic:0.12), (ERIBULIN:Colorectal cancer metastatic:0.36), (FESOTERODINE FUMARATE:Colorectal cancer metastatic:0.22), (FLUOXETINE\FLUOXETINE HYDROCHLORIDE:Colorectal cancer metastatic:0.47), (FLUTICASONE FUROATE:Colorectal cancer metastatic:0.2), (HEPARIN CALCIUM:Colorectal cancer metastatic:0.72), (HYDROCHLOROTHIAZIDE\TELMISARTAN:Colorectal cancer metastatic:0.1), (HYDROXYUREA:Colorectal cancer metastatic:0.3), (IBRUTINIB:Colorectal cancer metastatic:0.75), (IMATINIB MESYLATE:Colorectal cancer metastatic:0.76), (INFLIXIMAB-DYYB:Colorectal cancer metastatic:0.06), (INTERFERON ALFA-2A:Colorectal cancer metastatic:0.06), (IPRATROPIUM:Colorectal cancer metastatic:0.65), (ISOSORBIDE:Colorectal cancer metastatic:0.47), (LEDIPASVIR\SOFOSBUVIR:Colorectal cancer metastatic:0.28), (LETROZOLE:Colorectal cancer metastatic:0.29), (LEVALBUTEROL HYDROCHLORIDE:Colorectal cancer metastatic:0.3), (LEVOTHYROXINE SODIUM\POTASSIUM IODIDE:Colorectal cancer metastatic:0.1), (LINAGLIPTIN:Colorectal cancer metastatic:1.16), (LITHIUM CARBONATE:Colorectal cancer metastatic:0.4), (MECLIZINE\MECLIZINE HYDROCHLORIDE:Colorectal cancer metastatic:0.32), (MELATONIN:Colorectal cancer metastatic:0.12), (METAXALONE:Colorectal cancer metastatic:0.02), (METHYLPREDNISOLONE ACETATE:Colorectal cancer metastatic:0.19), (NADOLOL:Colorectal cancer metastatic:0.17), (NIACIN:Colorectal cancer metastatic:0.11), (NILOTINIB:Colorectal cancer metastatic:1.51), (OLAPARIB:Colorectal cancer metastatic:0.35), (OMEGA-3-ACID ETHYL ESTERS:Colorectal cancer metastatic:0.44), (OSIMERTINIB:Colorectal cancer metastatic:0.25), (OXCARBAZEPINE:Colorectal cancer metastatic:0.48), (OXYGEN:Colorectal cancer metastatic:0.45), (PAZOPANIB HYDROCHLORIDE:Colorectal cancer metastatic:0.83), (PEMETREXED:Colorectal cancer metastatic:0.74), (PEMETREXED DISODIUM:Colorectal cancer metastatic:0.36), (PENICILLIN:Colorectal cancer metastatic:0.53), (PRAMIPEXOLE DIHYDROCHLORIDE:Colorectal cancer metastatic:0.32), (PRIMIDONE:Colorectal cancer metastatic:0.33), (PROBIOTICS NOS:Colorectal cancer metastatic:0.98), (RADIATION THERAPY:Colorectal cancer metastatic:0.07), (RANOLAZINE:Colorectal cancer metastatic:0.46), (ROFLUMILAST:Colorectal cancer metastatic:0.71), (ROPINIROLE HYDROCHLORIDE:Colorectal cancer metastatic:0.1), (TERBINAFINE HYDROCHLORIDE:Colorectal cancer metastatic:0.02), (TETRACYCLINE:Colorectal cancer metastatic:0.37), (TIZANIDINE:Colorectal cancer metastatic:0.04), (TIZANIDINE HYDROCHLORIDE:Colorectal cancer metastatic:0.21), (TOCILIZUMAB:Colorectal cancer metastatic:0.01), (TOPIRAMATE:Colorectal cancer metastatic:1.37), (VALACYCLOVIR HYDROCHLORIDE:Colorectal cancer metastatic:0.37), (VANCOMYCIN HYDROCHLORIDE:Colorectal cancer metastatic:0.27), (VARENICLINE TARTRATE:Colorectal cancer metastatic:0.21), (VINORELBINE TARTRATE:Colorectal cancer metastatic:0.17), (DEXLANSOPRAZOLE:Completed suicide:0.09), (DIVALPROEX SODIUM:Completed suicide:0.45), (PREDNISONE:Completed suicide:0.17), (ZONISAMIDE:Completed suicide:0.3), (ETHOSUXIMIDE:Complex partial seizures:0.12), (NAPROXEN:Complex partial seizures:0.01), (ALCOHOL:Complex regional pain syndrome:0.02), (AMIODARONE:Complex regional pain syndrome:0.44), (ANASTROZOLE:Complex regional pain syndrome:0.55), (BISACODYL OR DOCUSATE SODIUM:Complex regional pain syndrome:0.2), (CARVEDILOL PHOSPHATE:Complex regional pain syndrome:0.37), (CEPHALEXIN:Complex regional pain syndrome:0.66), (CERTOLIZUMAB PEGOL:Complex regional pain syndrome:0.92), (CLOTRIMAZOLE:Complex regional pain syndrome:0.08), (COLCHICINE:Complex regional pain syndrome:0.3), (CORTISONE\HYDROCORTISONE:Complex regional pain syndrome:0.06), (DACLIZUMAB:Complex regional pain syndrome:0.15), (DALFAMPRIDINE:Complex regional pain syndrome:1.04), (DILTIAZEM:Complex regional pain syndrome:0.38), (DILTIAZEM HYDROCHLORIDE:Complex regional pain syndrome:0.9), (ENOXAPARIN:Complex regional pain syndrome:0.32), (ESOMEPRAZOLE MAGNESIUM\NAPROXEN:Complex regional pain syndrome:0.02), (ESTROGENS, CONJUGATED\MEDROXYPROGESTERONE ACETATE:Complex regional pain syndrome:0.02), (EVOLOCUMAB:Complex regional pain syndrome:0.61), (FLUOXETINE\FLUOXETINE HYDROCHLORIDE:Complex regional pain syndrome:0.39), (FLUTICASONE:Complex regional pain syndrome:0.12), (GLIMEPIRIDE:Complex regional pain syndrome:0.08), (GLIPIZIDE:Complex regional pain syndrome:0.08), (ILOPROST:Complex regional pain syndrome:0.05), (LABETALOL\LABETALOL HYDROCHLORIDE:Complex regional pain syndrome:0.29), (METHYLPREDNISOLONE ACETATE:Complex regional pain syndrome:0.21), (METRONIDAZOLE\METRONIDAZOLE HYDROCHLORIDE:Complex regional pain syndrome:0.39), (NIFEDIPINE:Complex regional pain syndrome:0.08), (NITROFURANTOIN:Complex regional pain syndrome:0.31), (PANTOPRAZOLE MAGNESIUM:Complex regional pain syndrome:0.17), (PAROXETINE\PAROXETINE HYDROCHLORIDE:Complex regional pain syndrome:0.54), (PRAMIPEXOLE DIHYDROCHLORIDE:Complex regional pain syndrome:0.61), (PRIMIDONE:Complex regional pain syndrome:0.59), (RANOLAZINE:Complex regional pain syndrome:0.43), (RISEDRONATE SODIUM:Complex regional pain syndrome:0.19), (SECUKINUMAB:Complex regional pain syndrome:0.01), (TERIFLUNOMIDE:Complex regional pain syndrome:0.99), (TETRACYCLINE:Complex regional pain syndrome:0.09), (UMECLIDINIUM BROMIDE\VILANTEROL TRIFENATATE:Complex regional pain syndrome:0.55), (BUMETANIDE:Computerised tomogram:0.57), (BUPROPION HYDROCHLORIDE:Computerised tomogram:0.4), (CALCIUM CARBONATE\CHOLECALCIFEROL:Computerised tomogram:0.83), (CANDESARTAN:Computerised tomogram:0.38), (CANDESARTAN CILEXETIL\HYDROCHLOROTHIAZIDE:Computerised tomogram:0.14), (CEPHALEXIN:Computerised tomogram:0.3), (CERTOLIZUMAB PEGOL:Computerised tomogram:0.71), (CLINDAMYCIN\CLINDAMYCIN PHOSPHATE:Computerised tomogram:0.55), (DASATINIB:Computerised tomogram:0.03), (DICLOFENAC:Computerised tomogram:0.01), (DIGOXIN:Computerised tomogram:1.01), (DILTIAZEM:Computerised tomogram:0.54), (DULOXETINE:Computerised tomogram:0.04), (ERGOCALCIFEROL:Computerised tomogram:0.11), (ESCITALOPRAM OXALATE:Computerised tomogram:0.25), (EVOLOCUMAB:Computerised tomogram:0.38), (GADOLINIUM:Computerised tomogram:0.08), (HYDROCODONE:Computerised tomogram:0.13), (IBRUTINIB:Computerised tomogram:1.25), (INDAPAMIDE\PERINDOPRIL:Computerised tomogram:0.1), (INSULIN LISPRO:Computerised tomogram:0.34), (LINAGLIPTIN:Computerised tomogram:0.05), (MELOXICAM:Computerised tomogram:0.25), (NAPROXEN SODIUM:Computerised tomogram:0.12), (NILOTINIB:Computerised tomogram:0.09), (NITROFURANTOIN:Computerised tomogram:0.12), (PROMETHAZINE\PROMETHAZINE HYDROCHLORIDE:Computerised tomogram:0.23), (RANOLAZINE:Computerised tomogram:0.2), (SOTALOL:Computerised tomogram:0.07), (TELMISARTAN:Computerised tomogram:0.13), (TRIAMCINOLONE ACETONIDE:Computerised tomogram:0.23), (AMPHETAMINE ASPARTATE\AMPHETAMINE SULFATE\DEXTROAMPHETAMINE SACCHARATE\DEXTROAMPHETAMINE SULFATE:Condition aggravated:0.05), (CIPROFLOXACIN HYDROCHLORIDE:Condition aggravated:0.22), (CLINDAMYCIN\CLINDAMYCIN PHOSPHATE:Condition aggravated:0.34), (CYCLOBENZAPRINE:Condition aggravated:0.05), (DULOXETINE:Condition aggravated:0.02), (ENALAPRIL:Condition aggravated:0.88), (EPINEPHRINE:Condition aggravated:0.15), (EVOLOCUMAB:Condition aggravated:0.13), (FERROUS SULFATE\FERROUS SULFATE, DRIED:Condition aggravated:0.14), (HYDROXYCHLOROQUINE DIPHOSPHATE:Condition aggravated:0.1), (IRON:Condition aggravated:0.26), (MELATONIN:Condition aggravated:0.13), (NEBIVOLOL:Condition aggravated:0.08), (NIFEDIPINE:Condition aggravated:0.26), (OXCARBAZEPINE:Condition aggravated:0.15), (PAROXETINE:Condition aggravated:0.11), (POLYETHYLENE GLYCOLS:Condition aggravated:0), (SACUBITRIL\VALSARTAN:Condition aggravated:0.03), (SECUKINUMAB:Condition aggravated:0.17), (SODIUM OXYBATE:Condition aggravated:0.13), (TERIFLUNOMIDE:Condition aggravated:0.33), (THALIDOMIDE:Condition aggravated:0.2), (VERAPAMIL HYDROCHLORIDE:Condition aggravated:0.01), (ZOLPIDEM\ZOLPIDEM TARTRATE:Condition aggravated:0.29), (ACETAMINOPHEN\CODEINE PHOSPHATE:Confusional state:0.25), (ALCOHOL:Confusional state:0.19), (AMPHOTERICIN B:Confusional state:0.03), (BUDESONIDE:Confusional state:0.28), (BUPRENORPHINE HYDROCHLORIDE:Confusional state:0.38), (BUPROPION HYDROCHLORIDE:Confusional state:0.86), (CALCIUM\VITAMIN D:Confusional state:0.01), (CEFTRIAXONE SODIUM:Confusional state:0.12), (CEPHALEXIN:Confusional state:0.39), (CILASTATIN SODIUM\IMIPENEM:Confusional state:0.05), (DEXLANSOPRAZOLE:Confusional state:0.1), (DICLOFENAC:Confusional state:0.36), (DILTIAZEM HYDROCHLORIDE:Confusional state:0.7), (EVEROLIMUS:Confusional state:0.09), (GADOLINIUM:Confusional state:0.05), (HYDROXYUREA:Confusional state:0.27), (IBRUTINIB:Confusional state:0.4), (MELATONIN:Confusional state:0.11), (METHYLPREDNISOLONE SODIUM SUCCINATE:Confusional state:0.58), (NILOTINIB:Confusional state:0.02), (OXCARBAZEPINE:Confusional state:0.82), (PAROXETINE\PAROXETINE HYDROCHLORIDE:Confusional state:0.07), (RANOLAZINE:Confusional state:0.16), (SACUBITRIL\VALSARTAN:Confusional state:0.54), (SOTALOL:Confusional state:0.1), (VARENICLINE TARTRATE:Confusional state:0.03), (NAPROXEN:Congenital cystic kidney disease:0.11), (MELATONIN:Congenital hypogammaglobulinaemia:0.08), (DILTIAZEM HYDROCHLORIDE:Congestive cardiomyopathy:0.37), (LERCANIDIPINE:Congestive cardiomyopathy:0.01), (ACETAMINOPHEN\BUTALBITAL\CAFFEINE:Conjunctivitis:0.07), (ALBUTEROL SULFATE\IPRATROPIUM BROMIDE:Conjunctivitis:0.28), (AMIODARONE:Conjunctivitis:0.69), (AMPHETAMINE ASPARTATE\AMPHETAMINE SULFATE\DEXTROAMPHETAMINE SACCHARATE\DEXTROAMPHETAMINE SULFATE:Conjunctivitis:0.01), (ANASTROZOLE:Conjunctivitis:0.93), (BICALUTAMIDE:Conjunctivitis: 0.14), (BISACODYL OR DOCUSATE SODIUM:Conjunctivitis:0.01), (BRIMONIDINE TARTRATE\TIMOLOL MALEATE:Conjunctivitis:0.07), (BUMETANIDE:Conjunctivitis:0.62), (BUPROPION:Conjunctivitis: 0.03), (CALCIUM CARBONATE\CHOLECALCIFEROL:Conjunctivitis:0.88), (CALCIUM\VITAMIN D:Conjunctivitis:0.34), (CANDESARTAN CILEXETIL\HYDROCHLOROTHIAZIDE:Conjunctivitis:0.3), (CHOLESTYRAMINE:Conjunctivitis: 0.18), (CILASTATIN SODIUM\IMIPENEM:Conjunctivitis:0.16), (CLONIDINE:Conjunctivitis:0.38), (DALFAMPRID INE:Conjunctivitis:0.26), (DASATINIB:Conjunctivitis:0.8), (DEXLANSOPRAZOLE:Conjunctivitis: 0.76), (DULOXETINE:Conjunctivitis:0.12), (ELETRIPTAN HYDROBROMIDE:Conjunctivitis:0.03), (ERGOCALCIFEROL:Conjunctivitis:0.43), (ESZOPIC LONE:Conjunctivitis:0.42), (EVEROLIMUS:Conjunctivitis:0.58), (EVOLOCUMAB:Conjunctivitis: 0.19), (EXEMESTANE:Conjunctivitis:0.04), (FENOFIBRATE:Conjunctivitis:0.22), (FLECAI NIDE:Conjunctivitis:0.04), (FLUTICASONE FUROATE\SALMETEROL XINAFOATE:Conjunctivitis:0.02), (GLIPIZIDE:Conjunctivitis:0.22), (INSULIN ASPART:Conjunctivitis:0.49), (LERCANIDIPINE:Conjunctivitis:0.06), (LOVASTATIN:Conjunctivitis: 0.7), (OLODATEROL HYDROCHLORIDE\TIOTROPIUM BROMIDE MONOHYDRATE:Conjunctivitis:0.2), (OXCARBAZEPINE:Conjunctivitis:0.25), (OXYGEN:Conjunctivitis: 0.41), (PANTOPRAZOLE MAGNESIUM:Conjunctivitis:0.12), (POLYETHYLENE GLYCOLS:Conjunctivitis:0.52), (PRIMIDONE:Conjunctivitis:0.01), (RANOLAZINE:Conjunctivitis: 0.04), (SACUBITRIL\VALSARTAN:Conjunctivitis:0.86), (SOTALOL:Conjunctivitis:0.15), (TOFACITINIB CITRATE:Conjunctivitis:0.12), (TRIAMTERENE:Conjunctivitis:0.22), (VANCOMYCIN HYDROCHLORIDE:Conjunctivitis:0.31), (VITAMIN B:Conjunctivitis:0.25), (WARFARIN SODIUM:Conjunctivitis:0.72), (CITALOPRAM HYDROBROMIDE:Conjunctivitis allergic:0.09), (NAPROXEN:Conjunctivitis allergic:0.16), (VERAPAMIL HYDROCHLORIDE:Conjunctivitis allergic:0.06), (ACETAMINOPHEN\BUTALBITAL\CAFFEINE:Connective tissue disorder:0.14), (ANASTROZOLE:Connective tissue disorder:0.24), (BISACODYL OR DOCUSATE SODIUM:Connective tissue disorder:0.03), (BISOPROLOL FUMARATE:Connective tissue disorder:0.44), (BUPROPION:Connective tissue disorder:0.01), (CALCITRIOL:Connective tissue disorder:0.14), (CARVEDILOL PHOSPHATE:Connective tissue disorder:0.08), (CLINDAMYCIN\CLINDAMYCIN PHOSPHATE:Connective tissue disorder:0.03), (DASATINIB:Connective tissue disorder:0.35), (DIPHENHYDRAMINE:Connective tissue disorder:0.27), (DIVALPROEX SODIUM:Connective tissue disorder:0.43), (ESTROGENS, CONJUGATED\MEDROXYPROGESTERONE ACETATE:Connective tissue disorder:0.15), (ESZOPICLONE:Connective tissue disorder:0.27), (EVEROLIMUS:Connective tissue disorder:0.13), (EVOLOCUMAB:Connective tissue disorder:1.12), (FENOFIBRATE:Connective tissue disorder:0.06), (FLUTICASONE FUROATE\VILANTEROL TRIFENATATE:Connective tissue disorder:0.03), (GLIPIZIDE:Connective tissue disorder:0.13), (HYDROCODONE:Connective tissue disorder:0.18), (HYDROXYZINE PAMOATE:Connective tissue disorder:0.14), (IBRUTINIB:Connective tissue disorder:0.81), (INDOMETHACIN:Connective tissue disorder:0.11), (ISOSORBIDE MONONITRATE:Connective tissue disorder:0.02), (LENALIDOMIDE:Connective tissue disorder:0.27), (LEVALBUTEROL HYDROCHLORIDE:Connective tissue disorder:0.2), (LEVOFLOXACIN:Connective tissue disorder:0.08), (LISDEXAMFETAMINE DIMESYLATE:Connective tissue disorder:0.1), (NAPROXEN:Connective tissue disorder:1.73), (NEBIVOLOL:Connective tissue disorder:0.16), (NILOTINIB:Connective tissue disorder:0.1), (OMEGA-3-ACID ETHYL ESTERS:Connective tissue disorder:0.03), (OXCARBAZEPINE:Connective tissue disorder:0.13), (PAROXETINE:Connective tissue disorder:0.04), (PONATINIB:Connective tissue disorder:0.06), (PRAMIPEXOLE DIHYDROCHLORIDE:Connective tissue disorder:0.17), (RANOLAZINE:Connective tissue disorder:0.46), (RIZATRIPTAN BENZOATE:Connective tissue disorder:0.12), (SACUBITRIL\VALSARTAN:Connective tissue disorder:0.06), (SUMATRIPTAN\SUMATRIPTAN SUCCINATE:Connective tissue disorder:0.44), (TAMSULOSIN HYDROCHLORIDE:Connective tissue disorder:0.11), (TEMAZEPAM:Connective tissue disorder:0.23), (TERIFLUNOMIDE:Connective tissue disorder:1.04), (ZOLPIDEM\ZOLPIDEM TARTRATE:Connective tissue disorder:0.36), (.BETA.-ACETYLDIGOXIN:Constipation:0.36), (.BETA.-HYDROXYISOVALERIC ACID\ARGININE\GLUTAMINE:Constipation:1.25), (2-HYDROXYETHYL METHACRYLATE:Constipation:0.22), (ACAMPROSATE:Constipation:0.67), (ACETAMINOP HEN\BROMISOVAL\DIHYDROCODEINE PHOSPHATE\DIPHENHYDRAMINE SALICYLATE\DYPHYLLINE\METHYLEPHEDRINE HYDROCHLORIDE, (+/−)-:Constipation:1.36), (ACETAMINOPHEN\BUTYLSCOPOLAMINE BROMIDE:Constipation:0.35), (ACETAMINOPHEN\CHLORPHENIRAMINE MALEATE\PSEUDOEPHEDRINE HYDROCHLORIDE:Constipation:0.58), (ACETYLCYSTEINE\BENZALKONIUM CHLORIDE\TUAMINOHEPTANE SULFATE:Constipation:0.08), (ACLIDINIUM BROMIDE\FORMOTEROL FUMARATE:Constipation:0.82), (ACRIVASTINE\PSEUDOEPHEDRINE HYDROCHLORIDE:Constipation:0.61), (ALBUTEROL\GUAIFENESIN:Constipation:1.3), (AL CLOMETASONE:Constipation:0.51), (ALISKIREN HEMIFUMARATE\HYDROCHLOROTHIAZIDE:Constipation:0.86), (ALISKIREN HEMIFUMARATE\VALSARTAN:Constipation:0.62), (ALISKIREN\HYDROCHLOROTHIAZ IDE:Constipation:0.55), (ALLANTOIN\BENZOCAINE\CAMPHOR (NATURAL)\DIMETHICONE\MENTHOL:Constipation:0.33), (ALUMINUM HYDROXIDE\DIMETHICONE\DIPHENHYDRAMINE\HYDROXIDE ION\LIDOCAINE:Constipation:0.82), (AMCINONIDE:Constipation:0.08), (AMIFOSTINE:Constipation: 0.03), (AMINO ACIDS\VITAMINS:Constipation:1.11), (AMINOBENZOIC ACID\CALCIUM PANTOTHENATE\CYSTINE\KERATIN\THIAMINE MONONITRATE:Constipation:0.33), (AMINOSALICYLIC ACID:Constipation:0.01), (AMLODIPINE MALEATE:Constipation:0.25), (AMLODIPINE\LOSARTAN:Constipation:0.31), (AMPHETA MINE:Constipation:0.08), (AMPHETAMINE SULFATE:Constipation:0.05), (AMPHOTERICIN B\DIMYRISTOYLPHOSPHATIDYLCHOLINE, DL-\DIMYRISTOYLPHOSPHATIDYLGLYCEROL, DL-:Constipation:0.38), (ANGIOTENSIN II:Constipation:0.3), (APPLE CIDER VINEGAR:Constipation:0.61), (ASCORBIC ACID/CHROMIUM/SELENIUM/VANADYL SULFATE:Constipation:0.8), (ASCORBIC ACID\BIOTIN\CALCIUM PANTOTHENATE\CYANOCOBALAMIN\FOLIC ACID\NIACINAMIDE\PYRIDOXINE HYDROCHLORIDE\RIBOFLAVIN\THIAMINE HYDROCHLORIDE:Constipation:0.38), (ASCORBIC ACID\CHONDROITIN SULFATE (BOVINE)\GLUCOSAMINE SULFATE\MANGANESE:Constipation:0.05), (ASCORBIC ACID\CYANOCOBALAMIN\FOLIC ACID\IRON:Constipation:0.07), (ASCORBIC ACID\FERROUS FUMARATE\FOLIC ACID:Constipation:0.35), (ASCORBIC ACID\IRON:Constipation:0.27), (ASPIRIN\CAFFEINE\SALICYLAMIDE:Constipation:0.71), (ATOVAQUONE\PROGUANIL HYDROCHLORIDE:Constipation:0.18), (ATRACURIUM BESYLATE:Constipation:1.16), (ATROPINE SULFATE\DIPHENOXYLATE:Constipation:0.49), (AVOBENZONE\HOMOSALATE\OCTIS ALATE:Constipation:1.09), (AZASETRON:Constipation:0.6), (AZITHROMYCIN:Constipation: 0.52), (AZITHROMYCIN MONOHYDRATE:Constipation:1.58), (AZITHROMYCIN OR METHOTREXATE SODIUM:Constipation:1.25), (BACITRACIN\NEOMYCIN SULFATE\POLYMYXIN B SULFATE:Constipation:0.02), (BACLOFEN\CYCLOBENZAPRINE\DICLOFENAC\GABAPE NTIN\TETRACAINE:Constipation:0.38), (BALSAM PERU\CASTOR OIL\TRYPSIN:Constipation:0.14), (BAMIFYLLINE HYDROCHLORIDE:Constipation:1.24), (BARNIDIPINE HYDROCHLORIDE:Constipation:0.09), (BECLOMETHASONE DIPROPIONATE\FORMOTEROL:Constipation:1.53), (BELATACEPT:Constipation:1.4), (BEN RALIZUMAB:Constipation:0.22), (BENZOCAINE\RESORCINOL:Constipation:0.46), (BENZO IC ACID\HYOSCYAMINE SULFATE\METHENAMINE\METHYLENE BLUE\PHENYL SALICYLATE:Constipation:0.26), (BEPOTASTINE\BEPOTASTINE BESILATE:Constipation:0.22), (BEPRIDIL HYDROCHLORIDE:Constipation:0.55), (BETAMETHASONE ACETATE\BETAMETHASONE SODIUM PHOSPHATE:Constipation:0.19), (BETAMETHASONE DIPROPIONATE\BETAMETHASONE SODIUM PHOSPHATE:Constipation:0.15), (BETAMETHASONE DIPROPIONATE\CALCIPOTRIENE HYDRATE:Constipation:0.54), (BETAMETHASONE DIPROPIONATE\GENTAMICIN SULFATE:Constipation:0.68), (BETAMETHASONE SODIUM PHOSPHATE\NEOMYCIN SULFATE:Constipation:1.97), (BETAMETHASONE VALERATE\FUSIDIC ACID:Constipation:0.43), (BETAMIPRON\PANIPENEM:Constipation:1.94), (BEXAROTENE: Constipation:0.54), (BILBERRY:Constipation:0.07), (BISMUTH:Constipation:0.5), (BLESELUM AB:Constipation:0.85), (BOCEPREVIR:Constipation:1.35), (BORON:Constipation:0.18), (BOTU LISM ANTITOXIN:Constipation:0.75), (BREXPIPRAZOLE:Constipation:0.66), (BRODALUMAB:Constipation: 0.19), (CAFFEINE\ERGOTAMINE TARTRATE:Constipation:0.07), (CAJUPUT OIL\EUCALYPTUS OIL\PEPPERMINT OIL:Constipation:0.77), (CALCIUM ASCORBATE:Constipation:0.07), (CALCIUM CARBONATE\ETIDRONATE DISODIUM:Constipation:0.76), (CALCIUM CHLORIDE\DEXTROSE MONOHYDRATE\HYDROCHLORIC ACID\MAGNESIUM CHLORIDE\SODIUM BICARBONATE\SODIUM CHLORIDE\SODIUM HYDROXIDE\SODIUM LACTATE:Constipation:0.04), (CALCIUM PANTOTHENATE\CUPRIC SULFATE\CYANOCOBALAMIN\FERROUS ASPARTO GLYCINATE\FERROUS FUMARATE\FOLIC ACID\MAGNESIUM SULFATE\NIACINAMIDE\PYRIDOXINE HYDROCHLORIDE\RIBOFLAVIN\SODIUM ASCORBATE\THIAMINE MONONITRATE\ZINC SULFATE:Constipation:0.09), (CALCIUM PANTOTHENATE\CYANOCOBALAMIN\NIACINAMIDE\PYRIDOXINE HYDROCHLORIDE\RIBOFLAVIN\THIAMINE HYDROCHLORIDE:Constipation:0.55), (CALCIUM PHOSPHATE:Constipation:0.62), (CALCIUM\CHOLECALCIFEROL\COPPER\MAGNESIU M\MANGANESE\ZINC:Constipation:0.29), (CALCIUM\ERGOCALCIFEROL:Constipation:0.24), (CANNABIDIOL:Constipation:0.94), (CAPECITABINE\OXALIPLATIN:Constipation:0.24), (CARBIDOPA\MELEVODOPA:Constipation:0.3), (CARBON DIOXIDE:Constipation:0.18), (CARBOPLATIN\PACLITAXEL:Constipation:0.12), (CEFMETA ZOLE:Constipation:1.47), (CEFMINOX SODIUM:Constipation:0.2), (CEFOPERAZONE\SULBACTAM:Constipation:0.31), (CEFPIRO ME:Constipation:0.2), (CEFPIROME SULFATE:Constipation:1.7), (CEFPROZIL:Constipation:2.43), (CEFTERAM PIVOXIL:Constipation:1.64), (CERIVASTATIN SODIUM:Constipation:0.68), (CERTOLIZUMAB:Constipation:0.06), (CHLORDIAZEPOXIDE\CLIDINIUM:Constipation:0.33), (CHLORMADINONE ACETATE\ETHINYL ESTRADIOL:Constipation:0.44), (CHLORPHENIRAMINE MALEATE\HYDROCODONE BITARTRATE\PSEUDOEPHEDRINE HYDROCHLORIDE:Constipation:0.15), (CHOLECALCIFEROL\GENISTEIN\ZINC GLYCINATE:Constipation:0.02), (CHOLIC ACID:Constipation:0.36), (CHOLINE\TRAZODONE HYDROCHLORIDE:Constipation:0.27), (CHROMIC CATION\CUPRIC CATION\FERROUS CATION\FLUORIDE ION\IODIDE ION\MANGANESE CATION (2+)\SODIUM SELENIDE\ZINC CATION:Constipation:0.17), (CHROMIC CHLORIDE\CHROMIUM:Constipation:1.16), (CHYMOTRYPSIN:Constipation:0.01), (CILAS TATIN SODIUM:Constipation:0.44), (CINEPAZIDE MALEATE:Constipation:0.01), (CIPROFLOXACIN\DEXAMETHASONE:Constipation:0.01), (CITICOLINE:Constipation:0.58), (CITRIC ACID MONOHYDRATE\POTASSIUM CITRATE:Constipation:0.39), (CITRIC ACID, 1-STEARYL ESTER\SODIUM CITRATE:Constipation:0.04), (CLIKSTAR:Constipation:1.05), (CLOCORTOLONE PIVALATE:Constipation:0.49), (CLOSTRIDIUM TETANI\CLOSTRIDIUM TETANI TOXOID ANTIGEN (FORMALDEHYDE INACTIVATED)\CORYNEBACTERIUM DIPHTHERIAE\CORYNEBACTERIUM DIPHTHERIAE TOXOID ANTIGEN (FORMALDEHYDE INACTIVATED):Constipation:0.55), (CLOXACILLIN SODIUM:Constipation:0.46), (COBALAMIN:Constipation:0.44), (CODEINE PHOSPHATE\DICLOFENAC SODIUM:Constipation:0.19), (CODEINE PHOSPHATE\GUAIFENESIN\PSEUDOEPHEDRINE HYDROCHLORIDE:Constipation:0.2), (COLCHICINE\DICYCLOMINE HYDROCHLORIDE:Constipation:0.97), (COPPER OR LEVONORGESTREL:Constipation:0.16), (COUMARIN:Constipation:0.24), (CROTALIDAE POLYVALENT IMMUNE FAB (OVINE):Constipation:1.1), (CYANOCOBALAMIN\PYRIDOXINE HYDROCHLORIDE\RIBOFLAVIN\SODIUM PANTOTHENATE\THIAMINE HYDROCHLORIDE\VITAMIN B COMPLEX:Constipation:0.74), (CYCLOPENTOLATE:Constipation:1.04), (CYCLOPHOSPHA MIDE\DOXORUBICIN\PREDNISONE\RITUXIMAB\VINCRISTINE:Constipation:0.41), (CY PROTERONE ACETATE\ETHINYL ESTRADIOL:Constipation:0.86), (CYTARABINE OCFOSFATE:Constipation:1.21), (DASABUVIR SODIUM MONOHYDRATE\OMBITASVIR HEMINONAHYDRATE\PARITAPREVIR DIHYDRATE\RITONAVIR:Constipation:0.47), (DEOXYRIBONUCLIEC ACID:Constipation:0.62), (DEXAMETHASONE\NEOMYCIN:Constipation:0.16), (DEXTROM ETHORPHAN HYDROBROMIDE\GUAIFENESIN\PSEUDOEPHEDRINE HYDROCHLORIDE:Constipation:0.12), (DICLOFENAC DIETHYLAMINE:Constipation:0.44), (DICLOXACILLIN SODIUM:Constipation:0.46), (DIENOGEST:Constipation:1.36), (DIETARY SUPPLEMENT\FLAVOCOXID:Constipation:0.1), (DIFLUNISAL:Constipation:1.61), (DIGITA LIS:Constipation:0.41), (DILAZEP DIHYDROCHLORIDE:Constipation:0.63), (DIPHENHYDRAMINE HYDROCHLORIDE\DOXYLAMINE SUCCINATE:Constipation:0.28), (DIPYRONE\PROMETHAZINE HYDROCHLORIDE:Constipation:1.81), (DOLASETRON:Constipation:0.49), (DOXIFLURIDI NE:Constipation:0.62), (DOXYLAMINE SUCCINATE\PYRIDOXINE HYDROCHLORIDE:Constipation:1.1), (DOXYLAMINE\PYRIDOXINE:Constipation:0.03), (D UPILUMAB:Constipation:0.29), (DYCLONINE HYDROCHLORIDE:Constipation:0.09), (DYDROGESTERONE\ESTRADIOL:Constipation:0.52), (EMTRICITABINE\TENOFOVIR:Constipation:0.06), (ENALAPRIL MALEATE\HYDROCHLOROTHIAZIDE:Constipation:1.63), (ENASIDENIB MESYLATE:Constipation:0.01), (ENFUVIRTIDE:Constipation:0.08), (EPACADOSTAT:Constipation: 0.65), (EPHEDRINE HYDROCHLORIDE\GUAIFENESIN:Constipation:0.88), (ESOMEPRAZOLE STRONTIUM:Constipation:0.18), (ESTRADIOL ACETATE:Constipation:0.57), (ESTRADIOL BENZOATE:Constipation:0.81), (ESTRADIOL HEMIHYDRATE:Constipation:0.67), (ESTRADIOL\LEVONORGESTREL:Constipation:0.34), (ESTROGENS, CONJUGATED SYNTHETIC B:Constipation:0.06), (ESTROGENS\METHYLTESTOSTERONE:Constipation:0.43), (ETHINY L ESTRADIOL\ETHYNODIOL:Constipation:0.42), (ETHINYL ESTRADIOL\ETHYNODIOL DIACETATE:Constipation:0.56), (ETHINYL ESTRADIOL\FERROUS FUMARATE\NORETHINDRONE:Constipation:0.46), (ETHINYL ESTRADIOL\FERROUS FUMARATE\NORETHINDRONE ACETATE:Constipation:0.36), (ETHINYL ESTRADIOL\GESTODENE:Constipation:0.96), (ETHINYL ESTRADIOL\LEVONORGESTREL ACETATE:Constipation:0.48), (ETIFOXINE:Constipation:0.06), (ETIMICIN SULFATE:Constipation:1.16), (EUCALYPTOL\MENTHOL\METHYL SALICYLATE\THYMOL:Constipation:0.48), (EVOFOSFAMIDE:Constipation:0.09), (FELODI PINE\METOPROLOL SUCCINATE:Constipation:0.5), (FENOPROFEN:Constipation:0.07), (FERRIC CARBOXYMALTOSE:Constipation:1.25), (FERROGLYCINE SULFATE:Constipation:0.09), (FERROUS BISGLYCINATE:Constipation:1.43), (FERROUS SULFATE\PROTEASE:Constipation:0.33), (FIMASARTAN:Constipation:0.43), (FINGOLIMO D:Constipation:0.55), (FLOMOXEF:Constipation:1.89), (FLUDEOXYGLUCOSE F-18:Constipation:0.24), (FLUMETHASONE PIVALATE:Constipation:0.12), (FLUNARIZINE:Constipation:0.2), (FLUNARIZINE HYDROCHLORIDE:Constipation:1.27), (FLUPENTIXOL:Constipation:0.91), (FLUPREDNIDE NE ACETATE\MICONAZOLE NITRATE:Constipation:0.17), (FOSAMPRENAVIR CALCIUM:Constipation:0.14), (FOSBRETABULIN TROMETHAMINE:Constipation:0.46), (FOSINOPRIL SODIUM\HYDROCHLOROTHIAZIDE:Constipation:1.1), (FURSULTIAMINE:Constipation:2.62), (GILTERITINIB:Constipation:0.23), (GLIMEPIRIDE\METFORMIN:Constipation:0.92), (G LIQUIDONE:Constipation:0.09), (GLUCONATE:Constipation:0.81), (GLYCOPYRRONIUM\F ORMOTEROL FUMARATE:Constipation:0.2), (GREEN TEA LEAF:Constipation:0.44), (GUAIFENESIN\PHENYLEPHRINE HYDROCHLORIDE:Constipation:0.81), (HALCINONIDE:Constipation:0.38), (HEMIN:Constipation: 0.03), (HERBALS\VITIS VINIFERA SEED:Constipation:0.39), (HESPERIDIN:Constipation:0.05), (HOMATROPINE HYDROBROMIDE:Constipation:0.25), (HOMATROPINE METHYLBROMIDE\HYDROCODONE BITARTRATE:Constipation:0.89), (HORSE CHESTNUT:Constipation:0.77), (HUMAN IMMUNOGLOBULIN G\HYALURONIDASE RECOMBINANT HUMAN:Constipation:1), (HYDRAZINE SULFATE:Constipation:0.6), (HYDROCHLOROTHIAZIDE\METOPROLOL TARTRATE:Constipation:0.62), (HYDROCORTISONE HEMISUCCINATE:Constipation:0.58), (HYDROQUINONE:Constipation:0.61), (HYDROXOC OBALAMIN\PYRIDOXINE HYDROCHLORIDE\THIAMINE HYDROCHLORIDE:Constipation:1.03), (ICATIBANT ACETATE:Constipation:0.34), (IDARUCIZUMAB:Constipation:1.13), (IFOSFAMIDE\MESNA:Constipation:0.06), (IMIGLUCERASE:Constipation:2.03), (IMIPRAMINE PAMOATE:Constipation:0.14), (INDAPAMIDE\PERINDOPRIL ERBUMINE:Constipation:0.31), (INDIGOTINDISULFONATE SODIUM:Constipation:0.56), (INDISETRON HYDROCHLORIDE:Constipation:0.54), (INFLIXIMAB-DYYB:Constipation:1.58), (INFLUENZA VIRUS VACCINE TRIVALENT TYPE A+B AFLURIA:Constipation:0.56), (INSULIN PORK\INSULIN PURIFIED PORK:Constipation:1.31), (INTERFERON ALFA-2A:Constipation:2.14), (INTERFERON ALFA-N1:Constipation:1.31), (INTERFERON GAMMA-1B:Constipation:2.35), (INVESTIGATIONAL BIOSIMILARS:Constipation:0.35), (IODINE\POTASSIUM IODIDE:Constipation:0.38), (IRBESARTAN\TRICHLORMETHIAZIDE:Constipation:0.92), (IR ON\VITAMIN B:Constipation:0.15), (ISOFLAVONES SOY:Constipation:0.29), (ISOFLURANE:Constipation:0.95), (ISONIAZID\PYRAZINAMIDE\R IFAMPIN:Constipation:0.5), (IVABRADINE HYDROCHLORIDE:Constipation:0.64), (IXAZOMIB CITRATE:Constipation:0.21), (KAOLIN:Constipation:0.32), (KETAZOLAM:Constipation:0.05), (LACTOBACILLUS ACIDOPHILUS\STREPTOCOCCUS FAECAIS:Constipation:0.76), (LANINAMIVIR:Constipation:0.54), (LANOLIN\MINERAL OIL\PETROLATUM:Constipation:0.9), (LANOLIN\PETROLATUM:Constipation:0.38), (LAN REOTIDE:Constipation:2.37), (LARONIDASE:Constipation:1.17), (LATANOPROST\TIMOLO L:Constipation:0.78), (LESINURAD:Constipation:0.02), (LESTAURTINIB:Constipation:0.1), (L ETROZOLE\RIBOCICLIB:Constipation:0.28), (LEVAMLODIPINE:Constipation:0.43), (LEVO BUPIVACAINE:Constipation:0.56), (LEVOMEFOLATE CALCIUM\METHYLCOBALAMIN\PYRIDOXAL 5-PHOSPHATE:Constipation:0.09), (LEVOSIMENDAN:Constipation:1.24), (LIPASE:Constipation: 0.93), (LITHIUM SULFATE:Constipation:0.29), (LOMERIZINE HYDROCHLORIDE:Constipation:0.16), (LOVASTATIN\NIACIN:Constipation:0.18), (LUMA CAFTOR:Constipation:1.23), (LUTETIUM LU-177:Constipation:1.33), (LYNESTRENOL:Constipation:0.35), (MAGNESIUM LACTATE\PYRIDOXINE HYDROCHLORIDE:Constipation:0.18), (MAGNESIUM SULFATE\POTASSIUM SULFATE\SODIUM SULFATE:Constipation:0.4), (MARIZOMIB:Constipation:0.12), (MECLIZINE MONOHYDROCHLORIDE\NIACIN:Constipation:0.63), (MECLOZINE DIHYDROCHLORIDE:Constipation:0.53), (MESTRANOL\NORETHINDRONE:Constipation: 0.53), (METFORMIN HYDROCHLORIDE\SAXAGLIPTIN:Constipation:1.55), (METHENAMINE MANDELATE:Constipation:1.24), (METHSUXIMIDE:Constipation:0.03), (METHYLPREDNIS OLONE OR METHYLPREDNISOLONE ACETATE:Constipation:0.51), (METRELEPTIN:Constipation:0.98), (METRONIDAZOLE BENZOATE:Constipation:0.37), (MILRINONE LACTATE:Constipation:0.64), (MINERALS\PROBIOTICS NOS\VITAMINS:Constipation:0.27), (MORPHINE\MORPHINE HYDROCHLORIDE\MORPHINE SULFATE\MORPHINE TARTRATE:Constipation:0.45), (NAFAMOSTAT:Constipation:0.66), (NATURAL LATEX RUBBER:Constipation:0.23), (NEDAPLATIN:Constipation:0.27), (NEISSERIA MENINGITIDIS GROUP A CAPSULAR POLYSACCHARIDE DIPHTHERIA TOXOID CONJUGATE ANTIGEN\NEISSERIA MENINGITIDIS GROUP C CAPSULAR POLYSACCHARIDE DIPHTHERIA TOXOID CONJUGATE ANTIGEN\NEISSERIA MENINGITIDIS GROUP W-135 CAPSULAR POLYSACCHARIDE DIPHTHERIA TOXOID CONJUGATE ANTIGEN\NEISSERIA MENINGITIDIS:Constipation:0.29), (NIACINAMIDE\PYRIDOXINE\RIBOFLAVIN\THIAMI NE:Constipation:0), (NIFURTOINOL:Constipation:0.84), (NIMETAZEPAM:Constipation:0.05), (NIMUSTINE:Constipation:0.79), (NITRIC OXIDE:Constipation:0.05), (NITROFURANTOIN MONOHYDRATE:Constipation:0.17), (OK-432:Constipation:1.49), (OLODATEROL:Constipation:0.31), (OMBITASVIR HEMINONAHYDRATE\PARITAPREVIR DIHYDRATE\RITONAVIR:Constipation:0.11), (OPICAPONE:Constipation:0.2), (OPRELVEKI N:Constipation:0.04), (ORTERONEL:Constipation:0.2), (OSTREA EDULIS SHELL:Constipation:0.09), (OXANDROLONE:Constipation:0.92), (PAMIDRONIC ACID:Constipation:0.53), (PANCURONIUM:Constipation:0.24), (PANIPENEM:Constipation:0.32), (PAPAVERINE HYDROCHLORIDE:Constipation:0.91), (PAPAYA:Constipation:0.72), (PARATHYROID HORMONE:Constipation:0.8), (PAROXETINE MESYLATE:Constipation:0.1), (PARSLEY:Constipation:0.16), (PASCOPYRUM SMITHII POLLEN:Constipation:0.08), (PAZUFLOXACIN:Constipation:0.48), (PEGINTERFERON LAMBDA-1A:Constipation:0.26), (PENTAERYTHRITOL TETRANITRATE:Constipation:0.98), (PENTOBARBITAL:Constipation:0.23), (PENTOSTATI N:Constipation:2.54), (PERAZINE:Constipation:0.13), (PHENELZINE:Constipation:0.4), (PHEN ELZINE SULFATE:Constipation:0.26), (PHENTOLAMINE:Constipation:0.89), (PHENYLEPHRINE HYDROCHLORIDE\PROMETHAZINE HYDROCHLORIDE:Constipation:0.08), (PHENYLEPHRINE HYDROCHLORIDE\WITCH HAZEL:Constipation:0.7), (PILSICAINIDE:Constipation:0.9), (PIPAMPERONE:Constipation:0.37), (PIRETANIDE:Constipation:0.8), (PIRIBEDIL:Constipation:0.88), (PLASMA PROTEIN FRACTION (HUMAN):Constipation:0.94), (PODOFILOX:Constipation:0.44), (POMEGRANATE:Constipati on:0.04), (POTASSIUM BITARTRATE:Constipation:0.13), (POTASSIUM BROMIDE:Constipation:0.55), (POTASSIUM NITRATE\SODIUM FLUORIDE:Constipation:0.04), (PREDNISOLONE ACETATE\SULFACETAMIDE SODIUM:Constipation:0.44), (PREDNISOLONE SODIUM METAZOATE:Constipation:0.48), (PROCAINE:Constipation:0.16), (PROCAINE HYDROCHLORIDE:Constipation:0.36), (PROPARACAINE:Constipation:0.34), (PROPIOMAZ INE:Constipation:0.21), (PROPOXYPHENE:Constipation:0.05), (PROTEASE:Constipation:0.94), (PSORALEN:Constipation:0.29), (PYRIDOXAL PHOSPHATE:Constipation:2.25), (QUINACRINE:Constipation:2.07), (QUINIDINE GLUCONATE:Constipation:0.16), (QUININE HYDROCHLORIDE:Constipation:0.33), (RALTITREXED:Constipation:0.27), (RESLIZUMAB: Constipation:0.29), (RILMAZAFONE:Constipation:0.27), (RILOTUMUMAB:Constipation:0.28), (RILPIVIRINE HYDROCHLORIDE:Constipation:1.77), (RIMANTADINE HYDROCHLORIDE:Constipation:0.23), (ROLAPITANT:Constipation:0.03), (RUPATADINE FUMARATE:Constipation:1.33), (SAFINAMIDE:Constipation:0.55), (SAFINAMIDE MESYLATE:Constipation:0.34), (SEBELIPASE ALFA:Constipation:0.41), (SERTACONAZOLE NITRATE:Constipation:0.49), (SERTINDOLE:Constipation:0.16), (SESAME OIL:Constipation:0.34), (SHARK CARTILAGE\SHARK, UNSPECIFIED:Constipation:0.22), (SIBUTRAMINE HYDROCHLORIDE:Constipation:1.63), (SILICON DIOXIDE:Constipation:1.84), (SILTUXIMAB:Constipation:0.15), (SIMEPREVIR SODIUM:Constipation:0.22), (SIVELESTAT SODIUM:Constipation:2.05), (SOBUZOXANE:Constipation:0.23), (SODIUM CHONDROITIN SULFATE:Constipation:0.27), (SODIUM FERRIC GLUCONATE COMPLEX:Constipation:0.56), (SODIUM HYDROXIDE:Constipation:0.02), (SODIUM HYPOCHLORITE:Constipation:0.16), (SODIUM NITROPRUSSIDE:Constipation:0.53), (SOLCOSERYL:Constipation:0.42), (SOMATREM:Constipation: 0.39), (STRONTIUM:Constipation:0), (STRONTIUM CHLORIDE SR-89:Constipation:0.26), (SULBUTIAMINE:Constipation:0.16), (SULFANILAMIDE:Constipation: 0.02), (TEDIZOLID:Constipation:0.11), (TEDUGLUTIDE:Constipation:1.86), (TEDUGLUTIDE\WATER:Constipation:2.74), (TELOTRISTAT ETHYL:Constipation:1.64), (TEMOCAPRIL:Constipation:1.12), (TENOXICAM:Constipation:0.13), (TESTOSTERONE UNDECANOATE:Constipation:2.33), (TETRACAINE HYDROCHLORIDE:Constipation:0.07), (THIAMYLAL:Constipation:0.4), (THIETHYLPERAZ INE MALATE:Constipation:0.2), (THIOPENTAL SODIUM:Constipation:0.25), (THIOTHIXENE HYDROCHLORIDE:Constipation:0.21), (THROMBOPOIETIN:Constipation:1.05), (TIAPRIDE:Constipation:0.89), (TIAPROFENIC ACID:Constipation:0.6), (TIMOLOL\TRAVOPROST:Constipation:0.85), (TINIDAZOLE:Constipation: 0.61), (TISAGENLECLEUCEL:Constipation:0.81), (TOFOGLIFLOZIN:Constipation:0.68), (TOREMIFENE:Constipation:0.91), (TRANYLCYPROMINE SULFATE:Constipation:0.19), (TRAPIDIL:Constipation:0.02), (TREPROSTINIL DIOLAMINE:Constipation:1.3), (TRIFLUOPERAZINE\TRIFLUOPERAZINE HYDROCHLORIDE:Constipation:0.31), (TRIMETHOBENZAMIDE:Constipation:0.37), (TRO METHAMINE:Constipation:0.08), (TRYPTOPHAN:Constipation:0.46), (UBENIMEX:Constipation: 0.7), (URACIL:Constipation:0.64), (URAPIDIL HYDROCHLORIDE:Constipation:0.09), (VALDECOXIB:Constipation:1.55), (VALPROATE MAGNESIUM:Constipation:0.07), (VINBLASTINE SULFATE:Constipation:0.95), (VITAMIN A PALMITATE:Constipation:0.65), (VORAPAXAR SULFATE:Constipation:0.38), (ZILEUTON:Constipation:2.44), (ZINC ACETATE:Constipation:1.2), (ZINC CITRATE:Constipation:1.03), (ZOLEDRONATE DISODIUM:Constipation:0.52), (ZUCLOPENTHIXOL DECANOATE:Constipation:1.58), (.ALPHA.1-PROTEINASE INHIBITOR HUMAN:Constipation prophylaxis:0.58), (ACETAMINOPHEN\CAFFEINE:Constipation prophylaxis:0.09), (ACETAMINOPHEN\OXYCODONE HYDROCHLORIDE\OXYCODONE TEREPHTHALATE:Constipation prophylaxis:0.12), (ACETAMINOPHEN\OXYCODONE\OXYCODONE HYDROCHLORIDE:Constipation prophylaxis:1.14), (ACETAMINOPHEN\PROPOXYPHENE NAPSYLATE:Constipation prophylaxis:0.13), (ACITRETIN:Constipation prophylaxis:0.07), (ACLIDINIUM BROMIDE:Constipation prophylaxis:0.06), (AGALSIDASE BETA:Constipation prophylaxis:0.55), (ALDESLEUKIN:Constipation prophylaxis:0.46), (ALISKIREN HEMIFUMARATE:Constipation prophylaxis:0.55), (AMINO ACIDS\CARBOHYDRATES\MINERALS\SOYBEAN OIL\VITAMINS:Constipation prophylaxis:0.06), (AMINO ACIDS\DEXTROSE\ELECTROLYTES NOS:Constipation prophylaxis:0.64), (AMISULPRIDE:Constipation prophylaxis:0.68), (AMLODIPINE BESYLATE\ATORVASTATIN CALCIUM:Constipation prophylaxis:0.28), (AMLODIPINE BESYLATE\HYDROCHLOROTHIAZIDE\VALSARTAN:Constipation prophylaxis:0.07), (AMLODIPINE BESYLATE\IRBESARTAN:Constipation prophylaxis:0.06), (AMLODIPINE BESYLATE\TELMISARTAN:Constipation prophylaxis:0.35), (AMLODIPINE\VALSARTAN:Constipation prophylaxis:0.68), (AMOXAPINE:Constipation prophylaxis:0.13), (ANTIHISTAMINES NOS:Constipation prophylaxis:0.18), (ASPIRIN\MAGNESIUM OXIDE:Constipation prophylaxis:0.23), (ATROPINE SULFATE\DIPHENOXYLATE HYDROCHLORIDE:Constipation prophylaxis:0.58), (ATROPINE\DIPHENOXYLATE:Constipation prophylaxis:0.1), (AZELNIDIPINE:Constipation prophylaxis:0.52), (BECLOMETHASONE:Constipation prophylaxis:0.35), (BELATACEPT:Constipation prophylaxis:0.18), (BENIDIPINE HYDROCHLORIDE:Constipation prophylaxis:0.23), (BETAHISTINE MESILATE:Constipation prophylaxis:0.21), (BETAMETHASONE DIPROPIONATE\CLOTRIMAZOLE:Constipation prophylaxis:0.03), (BETAMETHASONE\CALCIPOTRIENE:Constipation prophylaxis:0.25), (BLINATUMOMAB:Constipation prophylaxis:0.68), (BRIMONIDINE TARTRATE:Constipation prophylaxis:0.32), (BUPRENORPHINE HYDROCHLORIDE\NALOXONE HYDROCHLORIDE:Constipation prophylaxis:0.39), (CABOZANTINIB S-MALATE:Constipation prophylaxis:0.17), (CALCIUM CARBONATE\ERGOCALCIFEROL\RETINOL:Constipation prophylaxis:0.11), (CALCIUM\MAGNESIUM:Constipation prophylaxis:0.16), (CARBIDOPA:Constipation prophylaxis:0.02), (CARVEDILOL PHOSPHATE:Constipation prophylaxis:1.26), (CEFOTAXIME\CEFOTAXIME SODIUM:Constipation prophylaxis:0.11), (CERTOPARIN SODIUM:Constipation prophylaxis:0.12), (CHLORPHENIRAMINE\HYDROCODONE:Constipation prophylaxis:0.36), (CLOMIPRAMINE HYDROCHLORIDE:Constipation prophylaxis:0.37), (COD LIVER OIL:Constipation prophylaxis:0.11), (COLISTIMETHATE SODIUM:Constipation prophylaxis:0.17), (COLISTIN:Constipation prophylaxis:0.12), (CORTICOTROPIN:Constipation prophylaxis:0.08), (CORTISONE\HYDROCORTISONE:Constipation prophylaxis:1.55), (DACLATASVIR:Constipation prophylaxis:0.09), (DACLIZUMAB:Constipation prophylaxis:1.44), (DANAZOL:Constipation prophylaxis:0.53), (DANTROLENE SODIUM:Constipation prophylaxis:0.31), (DAPAGLIFLOZIN:Constipation prophylaxis:0.24), (DAPAGLIFLOZIN PROPANEDIOL:Constipation prophylaxis:0.29), (DEFLAZACORT:Constipation prophylaxis:0.69), (DESONIDE:Constipation prophylaxis:0.52), (DEVICE:Constipation prophylaxis:0.1), (DIGITOXIN:Constipation prophylaxis:0.67), (DILAZEP:Constipation prophylaxis:0.01), (DIMETHICONE\SILICON DIOXIDE\SIMETHICONE:Constipation prophylaxis:0.17), (DOLASETRON MESYLATE:Constipation prophylaxis:0.56), (DOXEPIN:Constipation prophylaxis:0.69), (ECONAZOLE:Constipation prophylaxis:0.77), (EDOXABAN TOSYLATE:Constipation prophylaxis:1.72), (ELOTUZUMAB:Constipation prophylaxis:1.1), (EMPAGLIFLOZIN\LINAGLIPTIN:Constipation prophylaxis:0.57), (ERIBULIN MESYLATE:Constipation prophylaxis:0.24), (ERLOTINIB:Constipation prophylaxis:0.45), (ESTROGENS:Constipation prophylaxis:0.11), (ETRETINATE:Constipation prophylaxis:0.7), (EVOLOCUMAB:Constipation prophylaxis:0.64), (FENOTEROL HYDROBROMIDE\IPRATROPIUM BROMIDE:Constipation prophylaxis:0.49), (FERROUS SULFATE\FOLIC ACID:Constipation prophylaxis:0.11), (FERROUS SULFATE\GLYCINE:Constipation prophylaxis:0.07), (FLECAINIDE:Constipation prophylaxis:1.91), (FLUDROCORTISONE ACETATE:Constipation prophylaxis:0.98), (FLUOROURACIL\LEUCOVORIN CALCIUM\OXALIPLATIN:Constipation prophylaxis:0.04), (FLUTAMIDE:Constipation prophylaxis:0.03), (FOLATE SODIUM:Constipation prophylaxis:0.04), (FONDAPARINUX:Constipation prophylaxis:0.25), (FONDAPARINUX SODIUM:Constipation prophylaxis:0.56), (FORMOTEROL:Constipation prophylaxis:0.9), (FORMOTEROL FUMARATE:Constipation prophylaxis:1.09), (FORMOTEROL FUMARATE\MOMETASONE FUROATE:Constipation prophylaxis:0.44), (FOSPHENYTOIN\FOSPHENYTOIN SODIUM:Constipation prophylaxis:0.24), (FULVESTRANT:Constipation prophylaxis:0.75), (GLYCOPYRROLATE\INDACATEROL MALEATE:Constipation prophylaxis:0.06), (HERBALS\SAW PALMETTO:Constipation prophylaxis:0.31), (HYDROCHLOROTHIAZIDE\VALSARTAN:Constipation prophylaxis:1.18), (HYDROCORTISONE ACETATE:Constipation prophylaxis:0.48), (HYDROXYCHLOROQUINE DIPHOSPHATE:Constipation prophylaxis:0.08), (HYOSCYAMINE:Constipation prophylaxis:0.31), (IBRITUMOMAB TIUXETAN:Constipation prophylaxis:0.2), (ILOPROST:Constipation prophylaxis:0.88), (IMIPENEM:Constipation prophylaxis:0.64), (INDAPAMIDE\PERINDOPRIL:Constipation prophylaxis:1.55), (INOTUZUMAB OZOGAMICIN:Constipation prophylaxis:0.15), (INTERFERON:Constipation prophylaxis:0.44), (INTERFERON ALFA-2A:Constipation prophylaxis:0.89), (INTERFERON ALFA-2B:Constipation prophylaxis:0.94), (INTERFERON GAMMA-1B:Constipation prophylaxis:0.24), (IODINE:Constipation prophylaxis:0.08), (LAPATINIB DITOSYLATE:Constipation prophylaxis:0.68), (LENVATINIB:Constipation prophylaxis:0.7), (LERCANIDIPINE HYDROCHLORIDE:Constipation prophylaxis:1.21), (LIMAPROST:Constipation prophylaxis:0.29), (LIOTHYRONINE:Constipation prophylaxis:0.09), (LIOTHYRONINE SODIUM:Constipation prophylaxis:0.23), (LISDEXAMFETAMINE DIMESYLATE:Constipation prophylaxis:0.32), (LITHIUM:Constipation prophylaxis:0.18), (LOMITAPIDE MESYLATE:Constipation prophylaxis:0.18), (LOTEPREDNOL ETABONATE:Constipation prophylaxis:0.09), (MAGNESIUM ASPARTATE\POTASSIUM ASPARTATE:Constipation prophylaxis:0.01), (MAXACALCITOL:Constipation prophylaxis:0.02), (MECLIZINE\MECLIZINE HYDROCHLORIDE:Constipation prophylaxis:0.66), (MEPOLIZUMAB:Constipation prophylaxis:0.28), (METAXALONE:Constipation prophylaxis:1.25), (METHENOLONE ENANTHATE:Constipation prophylaxis:0.79), (METHYLPREDNISOLONE ACETATE:Constipation prophylaxis:0.92), (MICONAZOLE NITRATE:Constipation prophylaxis:0.15), (NADOLOL:Constipation prophylaxis:0.6), (NEVIRAPINE:Constipation prophylaxis:0.38), (NICARDIPINE:Constipation prophylaxis:0.88), (NOREPINEPHRINE:Constipation prophylaxis:0.78), (OLODATEROL HYDROCHLORIDE\TIOTROPIUM BROMIDE MONOHYDRATE:Constipation prophylaxis:1.12), (PATIROMER:Constipation prophylaxis:0.01), (PEGINTERFERON ALFA-2B:Constipation prophylaxis:0.58), (PEGINTERFERON BETA-1A:Constipation prophylaxis:0.22), (PENTOSTATIN:Constipation prophylaxis:0.54), (PHENYTOIN SODIUM:Constipation prophylaxis:0.87), (PILOCARPINE:Constipation prophylaxis:0.21), (PILSICAINIDE HYDROCHLORIDE:Constipation prophylaxis:0.69), (PIMOBENDAN:Constipation prophylaxis:0.25), (PIPERACILLIN:Constipation prophylaxis:0.38), (PNEUMOCOCCAL VACCINE, POLYVALENT 23:Constipation prophylaxis:0.04), (POTASSIUM CITRATE\TRISODIUM CITRATE DIHYDRATE:Constipation prophylaxis:0.42), (PRAZOSIN:Constipation prophylaxis:0.3), (PROGESTERONE:Constipation prophylaxis:0.37), (PROPAFENONE:Constipation prophylaxis:0.25), (PYRAZINAMIDE:Constipation prophylaxis:0.75), (PYRIDOSTIGMINE BROMIDE:Constipation prophylaxis:0.29), (PYRIDOXINE:Constipation prophylaxis:0.51), (QUINAPRIL:Constipation prophylaxis:0.19), (RADIATION THERAPY:Constipation prophylaxis:1.28), (RETINOL:Constipation prophylaxis:0.77), (RILPIVIRINE HYDROCHLORIDE:Constipation prophylaxis:0.05), (RIVASTIGMINE:Constipation prophylaxis:0.43), (ROFLUMILAST:Constipation prophylaxis:1.34), (SARPOGRELATE:Constipation prophylaxis:0.99), (SELEXIPAG:Constipation prophylaxis:0.47), (SEVELAMER HYDROCHLORIDE:Constipation prophylaxis:0.9), (SIPULEUCEL-T:Constipation prophylaxis:0.13), (SITAGLIPTIN:Constipation prophylaxis:1.39), (SODIUM CITRATE:Constipation prophylaxis:0.07), (TAMOXIFEN CITRATE:Constipation prophylaxis:0.25), (TEDUGLUTIDE\WATER:Constipation prophylaxis:0.07), (TERIFLUNOMIDE:Constipation prophylaxis:0.69), (TETRACYCLINE:Constipation prophylaxis:0.58), (THROMBOMODULIN ALFA:Constipation prophylaxis:0.04), (TIMOLOL:Constipation prophylaxis:1.42), (TRABECTEDIN:Constipation prophylaxis:0.04), (TRAMETINIB DIMETHYL SULFOXIDE:Constipation prophylaxis:0.26), (TRANDOLAPRIL:Constipation prophylaxis:0.26), (TRIMIPRAMINE MALEATE:Constipation prophylaxis:0.15), (UMECLIDINIUM BROMIDE:Constipation prophylaxis:0.35), (VILAZODONE HYDROCHLORIDE:Constipation prophylaxis:0.24), (VINORELBINE\VINORELBINE TARTRATE:Constipation prophylaxis:0.42), (ZAFIRLUKAST:Constipation prophylaxis:0.23), (ZILEUTON:Constipation prophylaxis:0), (ZIV-AFLIBERCEPT:Constipation prophylaxis:0.42), (ZOLMITRIPTAN:Constipation prophylaxis:0.01), (.ALPHA.1-PROTEINASE INHIBITOR HUMAN:Contraception:1.15), (ACEBUTOLOL HYDROCHLORIDE:Contraception:0.06), (ACETAMINOPHEN\OXYCODONE\OXYCODON E HYDROCHLORIDE:Contraception:1.4), (ADO-TRASTUZUMAB EMTANSINE:Contraception:0.1), (ALBUMIN HUMAN:Contraception:0.06), (ALCOHOL\ALUMINUM CHLORIDE:Contraception:0.11), (ALFUZOSIN HYDROCHLORIDE:Contraception:0.35), (ALISKIREN HEMIFUMARATE:Contraception:0.2), (AMIKACIN:Contraception:0.47), (AMINO ACIDS\DEXTROSE\ELECTROLYTES NOS:Contraception:1.18), (AMIODARONE HYDROCHLORIDE:Contraception:1.17), (AMLODIPINE BESYLATE\ATORVASTATIN CALCIUM:Contraception:0.42), (AMLODIPINE BESYLATE\HYDROCHLOROTHIAZIDE\OLMESARTAN MEDOXOMIL:Contraception:0.71), (AMLODIPINE BESYLATE\HYDROCHLOROTHIAZIDE\VALSARTAN:Contraception:0.39), (AMLODIPINE BESYLATE\OLMESARTAN MEDOXOMIL:Contraception:0.38), (AMLODIPINE\VALSARTAN:Contraception:0.36),(AMMONIUM CHLORIDE\DEXTROMETHORPHAN\SODIUM CITRATE:Contraception:0.39), (AMPHETAMINE SULFATE:Contraception:0.35), (AMPHOTERICIN B:Contraception:0.87), (ARIPIPRAZOLE LAUROXIL:Contraception:0.59), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Contraception:0.1), (ASPARAGINASE:Contraception: 0.53), (AZITHROMYCIN OR METHOTREXATE SODIUM:Contraception:0.35), (BAZEDOXIFENE ACETATE\ESTROGENS, CONJUGATED:Contraception:0.46), (BECLOMETHASONE:Contraception:0.55), (BENZOYL PEROXIDE\ERYTHROMYCIN:Contraception:0.08), (BETAMETHASONE DIPROPIONATE\CALCIPOTRIENE HYDRATE:Contraception:0.34), (BETAMETHASONE SODIUM PHOSPHATE:Contraception:1.17), (BICALUTAMIDE:Contraception:0.61), (BIFIDOBACTERIUM ANIMALIS SUBSP. LACTIS:Contraception:0.09), (BILASTINE:Contraception:0.51), (BORTEZOMIB:Contraception: 0.48), (BRINZOLAMIDE:Contraception:0.13), (BROTIZOLAM:Contraception:0.02), (BUDESO NIDE\FORMOTEROL:Contraception:0.05), (BUPRENORPHINE\NALOXONE:Contraception: 0.43), (CALCITONIN:Contraception:0.63), (CALCITONIN SALMON:Contraception:1.05), (CALCIUM POLYCARBOPHIL:Contraception:0.27), (CALCIUM\MAGNESIUM:Contraception:0.92), (CA LCIUM\MAGNESIUM\ZINC:Contraception:1.02), (CANAGLIFLOZIN\METFORMIN HYDROCHLORIDE:Contraception:1.27), (CAPSAICIN:Contraception:1.22), (CARBIDOPA:Contraception: 0.23), (CARFILZOMIB:Contraception:0.45), (CARVEDILOL PHOSPHATE:Contraception:1.42), (CASPOFUNGIN:Contraception:0.17), (CEFEPIME HYDROCHLORIDE:Contraception:0.5), (CEFOTAXIME SODIUM:Contraception:1.26), (CEVIMELINE HYDROCHLORIDE:Contraception:0.32), (CHLORAMBUCIL:Contraception:0.09), (CHLORO THIAZIDE:Contraception:0.61), (CHONDROITIN SULFATE (BOVINE)\GLUCOSAMINE HYDROCHLORIDE:Contraception:1.14), (CHONDROITIN SULFATE (CHICKEN):Contraception:0.09), (CICLOPIROX OLAMINE:Contraception:0.15), (CILASTATIN SODIUM\IMIPENEM:Contraception:0.9), (CLOMIPRAMINE:Contraception:0.05), (CORTICO TROPIN:Contraception:0.93), (CURCUMIN:Contraception:0.06), (DANAZOL:Contraception:0.88), (DANTROLENE SODIUM:Contraception:0.16), (DAPAGLIFLOZIN:Contraception:0.22), (DEXAMETHASONE SODIUM PHOSPHATE:Contraception:0.16), (DEXTROMETHORPHAN HYDROBROMIDE\QUINIDINE SULFATE:Contraception:0.23), (DIFLUNISAL:Contraception:0.53), (DIMETHINDENE:Contraception: 0.06), (DIPYRONE\PROMETHAZINE HYDROCHLORIDE:Contraception:0.34), (DOLASETRON MESYLATE:Contraception:0.01), (DONEPEZIL:Contraception:0.72), (DRONEDARONE:Contraception: 0.11), (DUTASTERIDE\TAMSULOSIN HYDROCHLORIDE:Contraception:0.28), (DYDROGESTERONE:Contraception:0.49), (EDOX ABAN TOSYLATE:Contraception:0.57), (EFINACONAZOLE:Contraception:0.39), (EMPAGLIFLOZI N\LINAGLIPTIN:Contraception:0.97), (EMTRICITABINE\RILPIVIRINE\TENOFOVIR DISOPROXIL FUMARATE:Contraception:0.04), (EPLERENONE:Contraception:0.22), (ERGONOVINE\OXY TOCIN:Contraception:0.6), (ERTAPENEM SODIUM:Contraception:0.19), (ESLICARBAZEPINE:Contraception:0.12), (ESMOLOL HYDROCHLORIDE:Contraception:0.24), (ESTRADIOL BENZOATE:Contraception:0.02), (ESTRADIOL HEMIHYDRATE:Contraception:0.91), (ESTRADIOL\LEVONORGESTREL:Contraception:0.87), (EVOLOCUMAB:Contraception:1.53), (EXEMESTANE:Contraception:0.17), (FERRITIN:Contraception: 0.49), (FERROUS SULFATE\GLYCINE:Contraception:0.43), (FLUOCINOLONE ACETONIDE:Contraception:0.34), (FLUTICASONE PROPIONATE\FORMOTEROL FUMARATE:Contraception:0.2), (FOLLICLE STIMULATING HORMONE BETA POLYPEPTIDE\LUTROPIN ALFA:Contraception:0.28), (FONDAPARINUX:Contraception:0.09), (FOSFOMYCIN:Contraception: 0.25), (FOSPHENYTOIN\FOSPHENYTOIN SODIUM:Contraception:0.84), (FULVESTRANT:Contraception:0.26), (GLIPIZIDE\METFORM IN HYDROCHLORIDE:Contraception:0.13), (GLYCOPYRROLATE\INDACATEROL MALEATE:Contraception:0.03), (GLYCOPYRRONIUM:Contraception:0.59), (GLYCOPYRRO NIUM\INDACATEROL:Contraception:0.71), (HALOPERIDOL DECANOATE:Contraception:0.17), (HERBALS\SAW PALMETTO:Contraception:0.06), (HYALURONATE SODIUM:Contraception:0.08), (HYDROCHLOROTHIAZIDE\LOSARTAN:Contraception:0), (HYDROCHLOROTHIAZIDE\TELMISARTAN:Contraception:0.68), (IBRITUMOMAB TIUXETAN:Contraception:0.3), (ICOSAPENT ETHYL:Contraception:0.21), (ILOPROST:Contraception:0.51), (IMIPENEM:Contraception:0.37), (INDACATEROL:Contraception:0.32), (INTERFERON:Contraception:0.74), (INTERFERON GAMMA-1B:Contraception:0.37), (ISOFLURANE:Contraception:0.22), (ISOSORBIDE:Contraception:0.92), (IXEKIZUMAB:Contraception:0.1), (LERCANIDIPINE HYDROCHLORIDE:Contraception:1.45), (LEUPROLIDE ACETATE\NORETHINDRONE ACETATE:Contraception:0.1), (LINAGLIPTIN:Contraception:1.24), (LINEZOLID:Contraception: 0.63), (LUTEIN:Contraception:0.56), (MEGESTROL ACETATE:Contraception:0.22), (MENTHOL\METHYL SALICYLATE:Contraception:0.01), (MESNA:Contraception:0.02), (METOLAZONE:Contraception: 0.27), (METRELEPTIN:Contraception:0.24), (MOEXIPRIL HYDROCHLORIDE:Contraception:0.37), (MORPHINE SULFATE\NALTREXONE HYDROCHLORIDE:Contraception:0.58), (MOXONIDINE:Contraception:0.55), (NALOXONE HYDROCHLORIDE\OXYCODONE HYDROCHLORIDE:Contraception:1.53), (NALOXONE HYDROCHLORIDE\TILIDINE HYDROCHLORIDE:Contraception:1.36), (NALTREXONE HYDROCHLORIDE:Contraception:0.07), (NEOSTIGMINE:Contraception:0.57), (NOREPINEP HRINE:Contraception:0.27), (OCTREOTIDE:Contraception:0.45), (OLODATEROL HYDROCHLORIDE\TIOTROPIUM BROMIDE MONOHYDRATE:Contraception:0.61), (OMEGA-6 FATTY ACIDS:Contraception:0.14), (OSELTAMIVIR:Contraception:1.11), (OSPEMIFENE:Contraception: 3.13), (OXYMORPHONE:Contraception:0.4), (PANCRELIPASE\PANCRELIPASE AMYLASE\PANCRELIPASE LIPASE\PANCRELIPASE PROTEASE:Contraception:0), (PARICALCITOL:Contraception:0.13), (PEMETREXED DISODIUM:Contraception:0.51), (PENTAMIDINE ISETHIONATE:Contraception:0.26), (PENTAZOCINE LACTATE:Contraception:0.02), (PIRBUTEROL ACETATE:Contraception:0.34), (PITAVASTATIN CALCIUM:Contraception:0.42), (PIZOTYLINE:Contraception:0.47), (PRAMOXINE HYDROCHLORIDE:Contraception:0.1), (PREDNISOLONE SODIUM PHOSPHATE:Contraception:0.96), (PREDNISOLONE\PREDNISOLONE ACETATE\PREDNISOLONE SODIUM SUCCINATE:Contraception:0.08), (PREGNENOLONE:Contraception:0.34), (PRISTINAMYCI N:Contraception:0.19), (PROTRIPTYLINE HYDROCHLORIDE:Contraception:0.77), (QUINACRINE:Contraception:0.68), (QUINAPRIL:Contraception: 0.15), (QUINIDINE GLUCONATE:Contraception:0.06), (RALOXIFENE HYDROCHLORIDE:Contraception:1.04), (RANOLAZINE:Contraception:1.24), (RETINOL:Contraception: 1), (ROFLUMILAST:Contraception:1.45), (ROPIVACAINE:Contraception:0.03), (S ALMETEROL:Contraception:0.36), (SAW PALMETTO:Contraception:0.05), (SELEXIPAG:Contraception:0.34), (SITAGLIPTIN:Contraception: 0.76), (SODIUM PHOSPHATE, DIBASIC\SODIUM PHOSPHATE, MONOBASIC:Contraception:0.18), (SPIRAMYCIN:Contraception:0.09), (SUGAMMADEX:Contraception: 0.03), (SUNITINIB MALATE:Contraception:0.02), (TASIMELTEON:Contraception:0.33), (TAURINE:Contraception: 0.22), (TEDUGLUTIDE:Contraception:0.23), (TEDUGLUTIDE\WATER:Contraception:0.94), (TERAZOSIN HYDROCHLORIDE:Contraception:0.15), (TERAZOSIN\TERAZOSIN HYDROCHLORIDE:Contraception:0.09), (TERBUTALINE:Contraception:1.51), (TESTOSTER ONE UNDECANOATE:Contraception:0.97), (THIOPENTAL SODIUM:Contraception:0.26), (TIMOLOL:Contraception:0.64), (TINZAPARIN SODIUM:Contraception:0.53), (TIOTROPIUM:Contraception:0.6), (TRANDOLAPRIL:Contraception: 0.36), (TRIHEXYPHENIDYL:Contraception:0.27), (TRIHEXYPHENIDYL HYDROCHLORIDE:Contraception:0.1), (TRIMIPRAMINE MALEATE:Contraception:0.5), (UMECLIDINIUM BROMIDE:Contraception:0.49), (VINBLASTINE:Contraception:0.07), (YEAST:Contraception:00.65), (ZAFIRLUKAST:Contraception:0.88), (ZICONOTIDE ACETATE:Contraception:0.68), (CITALOPRAM HYDROBROMIDE:Contusion:0.01), (DILTIAZEM HYDROCHLORIDE:Contusion:0.3), (EVOLOCUMAB:Contusion:0.1), (RAMIPRIL:Contusion: 0.35), (TERIFLUNOMIDE:Contusion:0.2), (TORSEMIDE:Contusion:0.44), (VERAPAMIL HYDROCHLORIDE:Contusion:0.14), (ACETAMINOPHEN\CODEINE PHOSPHATE:Convulsion:0.72), (BISOPROLOL FUMARATE:Convulsion:0.34), (BRIVARACETAM:Convulsion:0.7), (BUMETANIDE:Convulsion: 0.06), (BUPRENORPHINE HYDROCHLORIDE:Convulsion:0.48), (BUSPIRONE HYDROCHLORIDE:Convulsion:0.69), (CALCITRIOL:Convulsion:0.27), (CALCIUM CARBONATE\CHOLECALCIFEROL:Convulsion:0.43), (CALCIUM CITRATE:Convulsion:0.06), (CEPHALEXIN:Convulsion:0.17), (CHOLESTYRAMINE:Convulsion: 0.04), (CLINDAMYCIN\CLINDAMYCIN PHOSPHATE:Convulsion:0.34), (COLCHICINE:Convulsion:0.24), (CORTISONE\HYDROCO RTISONE:Convulsion:0.41), (DALFAMPRIDINE:Convulsion:1.22), (DULOXETINE:Convulsion: 0.39), (ETHAMBUTOL HYDROCHLORIDE:Convulsion:0.93), (EVOLOCUMAB:Convulsion:0.78), (FERROUS SULFATE:Convulsion:0.74), (HYDROCHLOROTHIAZIDE\LISINOPRIL:Convulsion:0.23), (I BRUTINIB:Convulsion:0.99), (ILOPROST:Convulsion:0.12), (INDOMETHACIN:Convulsion:0), (LEDIPASVIR\SOFOSBUVIR:Convulsion:0.27), (LERCANIDIPINE:Convulsion:0.13), (MET AXALONE:Convulsion:0.4), (METHYLPREDNISOLONE ACETATE:Convulsion:0.64), (MOXIFLOXACIN HYDROCHLORIDE:Convulsion:0.09), (PANTOPRAZOLE:Convulsion:0.82), (PAROXETINE: Convulsion:0.19), (PRAMIPEXOLE DIHYDROCHLORIDE:Convulsion:0.58), (PRAVASTATIN:Convulsion:0.82), (RANITIDINE: Convulsion:0.02), (RANOLAZINE:Convulsion:0.47), (RISEDRONATE SODIUM:Convulsion:0.06), (SACUBITRIL\VALSARTAN:Convulsion:0.15), (SECUKINUMA B:Convulsion:0.31), (SUMATRIPTAN:Convulsion:0.05), (SUMATRIPTAN SUCCINATE:Convulsion:0.03), (TOCILIZUMAB:Convulsion:0.99), (TRIAMCINOLONE ACETONIDE:Convulsion:0.06), (VITAMIN B:Convulsion:0.19), (ACETAMINOPHEN\OXYCODONE\OXYCODONE HYDROCHLORIDE:Convulsion prophylaxis:0.12), (ALBUTEROL SULFATE\IPRATROPIUM BROMIDE:Convulsion prophylaxis:0.59), (ALCOHOL:Convulsion prophylaxis:0.54), (AMIKACIN:Convulsion prophylaxis:0.24), (AMIODARONE:Convulsion prophylaxis:0.95), (APREPITANT:Convulsion prophylaxis:0.38), (AZITHROMYCIN DIHYDRATE:Convulsion prophylaxis:0.34), (BENAZEPRIL HYDROCHLORIDE:Convulsion prophylaxis:0.54), (BISACODYL OR DOCUSATE SODIUM:Convulsion prophylaxis:0.54), (BLINATUMOMAB:Convulsion prophylaxis:0.06), (BRINZOLAMIDE:Convulsion prophylaxis:0.09), (BUMETANIDE:Convulsion prophylaxis:0.68), (BUPRENORPHINE HYDROCHLORIDE\NALOXONE HYDROCHLORIDE:Convulsion prophylaxis:0.34), (BUTYLSCOPOLAMINE BROMIDE:Convulsion prophylaxis:0.02), (CALCIUM CITRATE:Convulsion prophylaxis:0.25), (CANDESARTAN:Convulsion prophylaxis:1.22), (CANDESARTAN CILEXETIL\HYDROCHLOROTHIAZIDE:Convulsion prophylaxis:0.54), (CARISOPRODOL:Convulsion prophylaxis:0.15), (CARVEDILOL PHOSPHATE:Convulsion prophylaxis:0.38), (CEFTAZIDIME:Convulsion prophylaxis:0.16), (CERTOLIZUMAB PEGOL:Convulsion prophylaxis:0.96), (CHLORTHALIDONE:Convulsion prophylaxis:0.19), (CILASTATIN SODIUM\IMIPENEM:Convulsion prophylaxis:0.79), (CLARITHROMYCIN:Convulsion prophylaxis:0.23), (COLCHICINE:Convulsion prophylaxis:0.38), (CORTISONE\HYDROCORTISONE:Convulsion prophylaxis:0.36), (DACLIZUMAB:Convulsion prophylaxis:0.3), (DALFAMPRIDINE:Convulsion prophylaxis:0.91), (DESLORATADINE:Convulsion prophylaxis:0.17), (DEVICE:Convulsion prophylaxis:0.15), (DEXAMETHASONE ACETATE:Convulsion prophylaxis:0.1), (DEXLANSOPRAZOLE:Convulsion prophylaxis:1.04), (DICLOFENAC SODIUM:Convulsion prophylaxis:0.72), (DRONABINOL:Convulsion prophylaxis:0.51), (ELETRIPTAN HYDROBROMIDE:Convulsion prophylaxis:0.4), (ENALAPRIL MALEATE:Convulsion prophylaxis:1.1), (EPIRUBICIN:Convulsion prophylaxis:0.21), (ESTROGENS, CONJUGATED\MEDROXYPROGESTERONE ACETATE:Convulsion prophylaxis:0.17), (ETHAMBUTOL HYDROCHLORIDE:Convulsion prophylaxis:0.94), (EVOLOCUMAB:Convulsion prophylaxis:0.31), (FESOTERODINE FUMARATE:Convulsion prophylaxis:0.15), (FINGOLIMOD HYDROCHLORIDE:Convulsion prophylaxis:0.54), (FLECAINIDE:Convulsion prophylaxis:0.37), (FLUTICASONE:Convulsion prophylaxis:0.15), (FLUTICASONE FUROATE\VILANTEROL TRIFENATATE:Convulsion prophylaxis:0.09), (FLUTICASONE\FLUTICASONE PROPIONATE:Convulsion prophylaxis:0.03), (GLIPIZIDE:Convulsion prophylaxis:0.56), (IBRUTINIB:Convulsion prophylaxis:1.18), (IMATINIB MESYLATE:Convulsion prophylaxis:1.05), (INDAPAMIDE:Convulsion prophylaxis:0.26), (INDAPAMIDE\PERINDOPRIL:Convulsion prophylaxis:0.1), (INFLUENZA VIRUS VACCINE:Convulsion prophylaxis:0.06), (INTERFERON ALFA-2B:Convulsion prophylaxis:0.03), (IPRATROPIUM:Convulsion prophylaxis:0.47), (IRBESARTAN:Convulsion prophylaxis:0.57), (ISOSORBIDE:Convulsion prophylaxis:0.36), (IXAZOMIB:Convulsion prophylaxis:0), (LEDIPASVIR\SOFOSBUVIR:Convulsion prophylaxis:0.42), (LERCANIDIPINE HYDROCHLORIDE:Convulsion prophylaxis:0), (LETROZOLE:Convulsion prophylaxis:0.4), (LISDEXAMFETAMINE DIMESYLATE:Convulsion prophylaxis:0.72), (LOVASTATIN:Convulsion prophylaxis:0.71), (METHENAMINE HIPPURATE:Convulsion prophylaxis:0.16), (METHYLPREDNISOLONE ACETATE:Convulsion prophylaxis:0.42), (MINOCYCLINE\MINOCYCLINE HYDROCHLORIDE:Convulsion prophylaxis:0.67), (MYCOPHENOLATE MOFETIL\MYCOPHENOLATE MOFETIL HYDROCHLORIDE:Convulsion prophylaxis:0.16), (NABUMETONE:Convulsion prophylaxis:0.01), (NALOXONE HYDROCHLORIDE\OXYCODONE HYDROCHLORIDE:Convulsion prophylaxis:0.44), (NILOTINIB:Convulsion prophylaxis:1.36), (OLAPARIB:Convulsion prophylaxis:0.1), (OSELTAMIVIR:Convulsion prophylaxis:0.3), (OXYGEN:Convulsion prophylaxis:0.53), (PENICILLIN:Convulsion prophylaxis:0.45), (PONATINIB:Convulsion prophylaxis:0.5), (PYRIDOXINE HYDROCHLORIDE:Convulsion prophylaxis:0.19), (RABEPRAZOLE:Convulsion prophylaxis:0.19), (REGORAFENIB:Convulsion prophylaxis:0.06), (RIZATRIPTAN BENZOATE:Convulsion prophylaxis:0.53), (ROFLUMILAST:Convulsion prophylaxis:0.06), (ROPINIROLE HYDROCHLORIDE:Convulsion prophylaxis:0.17), (SECUKINUMAB:Convulsion prophylaxis:0.15), (SITAGLIPTIN:Convulsion prophylaxis:0.01), (SOTALOL:Convulsion prophylaxis:0.58), (SUMATRIPTAN\SUMATRIPTAN SUCCINATE:Convulsion prophylaxis:0.62), (TIMOLOL:Convulsion prophylaxis:0.53), (TOCILIZUMAB:Convulsion prophylaxis:0.13), (UMECLIDINIUM BROMIDE\VILANTEROL TRIFENATATE:Convulsion prophylaxis:0.09), (VARENICLINE TARTRATE:Convulsion prophylaxis:1.15), (ZOPICLONE:Convulsion prophylaxis:0.5), (ACETAMINOPHEN\BUTALBITAL\CAFFEINE:Cor pulmonale chronic:0.47), (AMPHETAMINE ASPARTATE\AMPHETAMINE SULFATE\DEXTROAMPHETAMINE SACCHARATE\DEXTROAMPHETAMINE SULFATE:Cor pulmonale chronic:0.39),(ARMODAFINIL:Cor pulmonale chronic:0.25), (BISOPROLOL FUMARATE:Cor pulmonale chronic:0.57), (CANDESARTAN:Cor pulmonale chronic:0.1), (CAPTOPRIL:Cor pulmonale chronic:0.25), (CARVEDILOL PHOSPHATE:Cor pulmonale chronic:0.57), (CERTOLIZUMAB PEGOL:Cor pulmonale chronic:0.37), (DARBEPOETIN ALFA:Cor pulmonale chronic:0.07), (DOXAZOSIN MESYLATE:Cor pulmonale chronic:0.06), (ELETRIPTAN HYDROBROMIDE:Cor pulmonale chronic:0.3), (ESOMEPRAZOLE:Cor pulmonale chronic:0.17), (EVEROLIMUS:Cor pulmonale chronic:0.32), (EVOLOCUMAB:Cor pulmonale chronic:0.51), (FLUTICASONE:Cor pulmonale chronic:0.57), (HYDROCHLOROTHIAZIDE\LOSARTAN POTASSIUM:Cor pulmonale chronic:0.01), (HYDROCHLOROTHIAZIDE\VALSARTAN:Cor pulmonale chronic:0.09), (IBRUTINIB:Cor pulmonale chronic:0.81), (LIDOCAINE HYDROCHLORIDE:Cor pulmonale chronic:0.17), (LISDEXAMFETAMINE DIMESYLATE:Cor pulmonale chronic:0.03), (METAXALONE:Cor pulmonale chronic:0.59), (METHYLPREDNISOLONE ACETATE:Cor pulmonale chronic:0.08), (METHYLPREDNISOLONE SODIUM SUCCINATE:Cor pulmonale chronic:0.15), (METRONIDAZOLE\METRONIDAZOLE HYDROCHLORIDE:Cor pulmonale chronic:0.12), (MOXIFLOXACIN HYDROCHLORIDE:Cor pulmonale chronic:0.23), (NEBIVOLOL:Cor pulmonale chronic:0.24), (NORTRIPTYLINE:Cor pulmonale chronic:0.14), (OMEGA-3-ACID ETHYL ESTERS:Cor pulmonale chronic:0.49), (PANTOPRAZOLE:Cor pulmonale chronic:1.24), (PANTOPRAZOLE MAGNESIUM:Cor pulmonale chronic:0.42), (PONATINIB:Cor pulmonale chronic:0.03), (PRIMIDONE:Cor pulmonale chronic:0.64), (PROBIOTICS NOS:Cor pulmonale chronic:0.31), (RANOLAZINE:Cor pulmonale chronic:1.17), (SACUBITRIL\VALSARTAN:Cor pulmonale chronic:0.02), (SODIUM OXYBATE:Cor pulmonale chronic:0.38), (TERIFLUNOMIDE:Cor pulmonale chronic:0.29), (TIMOLOL:Cor pulmonale chronic:0.13), (TIZANIDINE HYDROCHLORIDE:Cor pulmonale chronic:0.56), (VARENICLINE TARTRATE:Cor pulmonale chronic:0.48), (VITAMIN B:Cor pulmonale chronic:0.46), (ACENOCOUMAROL:Coronary arterial stent insertion:0.11), (AMPHETAMINE ASPARTATE\AMPHETAMINE SULFATE\DEXTROAMPHETAMINE SACCHARATE\DEXTROAMPHETAMINE SULFATE:Coronary arterial stent insertion:0.36), (CALCIUM ACETATE:Coronary arterial stent insertion:0.35), (CANDESARTAN CILEXETIL\HYDROCHLOROTHIAZIDE:Coronary arterial stent insertion:0.33), (CLOTRIMAZOLE:Coronary arterial stent insertion:0.13), (DAPAGLIFLOZIN PROPANEDIOL:Coronary arterial stent insertion:0.05), (DASATINIB:Coronary arterial stent insertion:0.02), (DEVICE:Coronary arterial stent insertion:0.51), (DICLOFENAC:Coronary arterial stent insertion:0.66), (DIVALPROEX SODIUM:Coronary arterial stent insertion:0.06), (ELETRIPTAN HYDROBROMIDE:Coronary arterial stent insertion:0.05), (FLECAINIDE:Coronary arterial stent insertion:0.5), (FLUTICASONE:Coronary arterial stent insertion:0.34), (HYDROCHLOROTHIAZIDE\IRBESARTAN:Coronary arterial stent insertion:0.06), (IBRUTINIB:Coronary arterial stent insertion:0.83), (ILOPROST:Coronary arterial stent insertion:0.25), (INDAPAMIDE:Coronary arterial stent insertion:0.08), (LATANOPROST:Coronary arterial stent insertion:0.15), (LEDIPASVIR\SOFOSBUVIR:Coronary arterial stent insertion:0.71), (LERCANIDIPINE HYDROCHLORIDE:Coronary arterial stent insertion:0.01), (LISDEXAMFETAMINE DIMESYLATE:Coronary arterial stent insertion:0.21), (METAXALONE:Coronary arterial stent insertion:0.24), (METRONIDAZOLE:Coronary arterial stent insertion:0.59), (NILOTINIB:Coronary arterial stent insertion:0.3), (NITROFURANTOIN:Coronary arterial stent insertion:0.19), (NYSTATIN:Coronary arterial stent insertion:0.17), (OLANZAPINE:Coronary arterial stent insertion:0.05), (PANTOPRAZOLE MAGNESIUM:Coronary arterial stent insertion:0.18), (PAROXETINE\PAROXETINE HYDROCHLORIDE:Coronary arterial stent insertion:0.04), (POLYETHYLENE GLYCOLS:Coronary arterial stent insertion:0.49), (RABEPRAZOLE SODIUM:Coronary arterial stent insertion:0.43), (SECUKINUMAB:Coronary arterial stent insertion:0.17), (TERIFLUNOMIDE:Coronary arterial stent insertion:0.33), (TOCILIZUMAB:Coronary arterial stent insertion:0.15), (TOFACITINIB CITRATE:Coronary arterial stent insertion:0.02), (UMECLIDINIUM BROMIDE\VILANTEROL TRIFENATATE:Coronary arterial stent insertion:0.2), (VARENICLINE TARTRATE:Coronary arterial stent insertion:0.29), (VITAMIN B:Coronary arterial stent insertion:0.46), (ANASTROZOLE:Coronary artery bypass:0.3), (CALCITRIOL:Coronary artery bypass:0.25), (CARFILZOMIB:Coronary artery bypass:0.01), (CLINDAMYCIN\CLINDAMYCIN PHOSPHATE:Coronary artery bypass:0.14), (DASATINIB:Coronary artery bypass:0.29), (DEVICE:Coronary artery bypass:0.2), (DEXLANSOPRAZOLE:Coronary artery bypass:0.68), (DIPHENHYDRAMINE:Coronary artery bypass:0.16), (ENALAPRIL MALEATE:Coronary artery bypass:0.02), (ESZOPICLONE:Coronary artery bypass:0.03), (EVEROLIMUS:Coronary artery bypass:0.23), (FLUTICASONE PROPIONATE:Coronary artery bypass:0.75), (HYDROCHLOROTHIAZIDE\LISINOPRIL:Coronary artery bypass:0.24), (HYDROCODONE:Coronary artery bypass:0.11), (INDAPAMIDE\PERINDOPRIL:Coronary artery bypass:0.27), (INSULIN DETEMIR:Coronary artery bypass:0.27), (LERCANIDIPINE:Coronary artery bypass:0.56), (METHYLPREDNISOLONE SODIUM SUCCINATE:Coronary artery bypass:0.37), (NEBIVOLOL:Coronary artery bypass:0.6), (NILOTINIB:Coronary artery bypass:0.13), (OXYGEN:Coronary artery bypass:0.42), (PRAMIPEXOLE DIHYDROCHLORIDE:Coronary artery bypass:0.04), (PRAVASTATIN SODIUM:Coronary artery bypass:0.72), (TERIFLUNOMIDE:Coronary artery bypass:0.23), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Coronary artery disease:0.26), (ABOBOTULINUMTOXINA:Coronary artery disease:0.08), (ACETAMINOPHEN\CAFFEINE\OPIUM:Coronary artery disease:0.6), (ACETAMINOPHEN\DIPHENHYDRAMINE HYDROCHLORIDE:Coronary artery disease:0.03), (ACETAMINOPHEN\PROPOXYPHENE NAPSYLATE:Coronary artery disease:0.16), (ACETAMINOPHEN\TRAMADOL:Coronary artery disease:0.85), (ACTIVATED CHARCOAL:Coronary artery disease:0.73), (ADO-TRASTUZUMAB EMTANSINE:Coronary artery disease:1.59), (AGALSIDASE BETA:Coronary artery disease:1.53), (ALCOHOL:Coronary artery disease:0.8), (ALDESLEUKIN:Coronary artery disease:0.06), (ALISKIREN HEMIFUMARATE\HYDROCHLOROTHIAZIDE:Coronary artery disease:0.09), (AMIKACIN:Coronary artery disease:0.09), (AMINO ACIDS\DEXTROSE\ELECTROLYTES NOS:Coronary artery disease:0.55), (AMISULPRIDE:Coronary artery disease:1.29), (AMMONIUM CHLORIDE\DEXTROMETHORPHAN\SODIUM CITRATE:Coronary artery disease:0.03), (AMOXICILLIN\CLARITHROMYCIN\ESOMEPRAZOLE:Coronary artery disease:0.14), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Coronary artery disease:0.38), (ASUNAPREVIR:Coronary artery disease:0.18), (ATOVAQUONE:Coronary artery disease:0.47), (AVANAFIL:Coronary artery disease:0.05), (AXITINIB:Coronary artery disease:1.33), (BAMIFYLLINE HYDROCHLORIDE:Coronary artery disease:0.01), (BECLOMETHASONE DIPROPIONATE\FORMOTEROL:Coronary artery disease:0.06), (BENIDIPINE:Coronary artery disease:0.02), (BENZTROPINE:Coronary artery disease:0.03), (BENZTROPINE MESYLATE:Coronary artery disease:0.07), (BETAMETHASONE DIPROPIONATE\CALCIPOTRIENE:Coronary artery disease:0.02), (BETHANECHOL CHLORIDE:Coronary artery disease:0.44), (BILASTINE:Coronary artery disease:0.43), (BIPERIDEN HYDROCHLORIDE:Coronary artery disease:0.01), (BLINATUMOMAB:Coronary artery disease:0.43), (BOCEPREVIR:Coronary artery disease:0.23), (BUDESONIDE\FORMOTEROL FUMARATE:Coronary artery disease:1.72), (CABOZANTINIB S-MALATE:Coronary artery disease:0.21), (CALCITONIN SALMON:Coronary artery disease:1.3), (CALCIUM CHLORIDE\DEXTROSE MONOHYDRATE\MAGNESIUM CHLORIDE\SODIUM CHLORIDE\SODIUM LACTATE:Coronary artery disease:0.7), (CALCIUM PANTOTHENATE\CYANOCOBALAMIN\NIACINAMIDE\PYRIDOXINE HYDROCHLORIDE\RIBOFLAVIN\THIAMINE MONONITRATE:Coronary artery disease:0.13), (CALCIUM\MAGNESIUM:Coronary artery disease:0.64), (CALCIUM\MAGNESIUM\ZINC:Coronary artery disease:0.94), (CARBOHYDRATES\FATTY ACIDS\MINERALS\PROTEIN\VITAMINS:Coronary artery disease:0.31), (CARBOMER:Coronary artery disease:0.34), (CARTEOLOL:Coronary artery disease:0.01), (CASANTHRANOL\DOCUSATE SODIUM:Coronary artery disease:0.34), (CEFTAZIDIME:Coronary artery disease:0.64), (CELIPROLOL:Coronary artery disease:0.33), (CERITINIB:Coronary artery disease:0.72), (CETIRIZINE HYDROCHLORIDE\PSEUDOEPHEDRINE HYDROCHLORIDE:Coronary artery disease:0.4), (CHLORPHENIRAMINE\HYDROCODONE:Coronary artery disease:0.41), (CHONDROITIN SULFATE A\GLUCOSAMINE:Coronary artery disease:0.38), (CINACALCET:Coronary artery disease:0.3), (CLOBAZAM:Coronary artery disease:0.53), (CLOXAZOLAM:Coronary artery disease:0.06), (COAGULATION FACTOR IX HUMAN\COAGULATION FACTOR VII HUMAN\COAGULATION FACTOR X HUMAN\PROTEIN C\PROTEIN S HUMAN\PROTHROMBIN:Coronary artery disease:0.08), (CYAMEMAZINE:Coronary artery disease:0.4), (CYPROHEPTADINE:Coronary artery disease:0.41), (DABRAFENIB:Coronary artery disease:0.92), (DANTROLENE SODIUM:Coronary artery disease:0.3), (DEFLAZACORT:Coronary artery disease:1.48), (DEGARELIX:Coronary artery disease:0.11), (DEXAMETHASONE ACETATE:Coronary artery disease:0.76), (DEXTROAMPHETAMINE SULFATE:Coronary artery disease:0.41), (DEXTROSE\HEPARIN:Coronary artery disease:0.34), (DOBUTAMINE HYDROCHLORIDE:Coronary artery disease:0.36), (DOCETAXEL:Coronary artery disease:1.33), (DOLASETRON MESYLATE:Coronary artery disease:0.66), (DORZOLAMIDE HYDROCHLORIDE:Coronary artery disease:0.66), (DOXEPIN HYDROCHLORIDE:Coronary artery disease:0.94), (ECONAZOLE:Coronary artery disease:1.57), (ELOTUZUMAB:Coronary artery disease:0.8), (ELTROMBOPAG OLAMINE:Coronary artery disease:0.75), (ENALAPRIL MALEATE\LERCANIDIPINE HYDROCHLORIDE:Coronary artery disease:0.27), (ERIBULIN:Coronary artery disease:0.24), (ESOMEPRAZOLE MAGNESIUM\NAPROXEN:Coronary artery disease:0.24), (ESOMEPRAZOLE SODIUM:Coronary artery disease:0.04), (ESTRIOL:Coronary artery disease:0.09), (FERRIC HYDROXIDE:Coronary artery disease:0.67), (FEXOFENADINE\PSEUDOEPHEDRINE:Coronary artery disease:0.47), (FLUCLOXACILLIN:Coronary artery disease:0.07), (FLURBIPROFEN:Coronary artery disease:0.09), (FORMOTEROL FUMARATE\MOMETASONE FUROATE:Coronary artery disease:0.89), (FOSFOMYCIN:Coronary artery disease:0.15), (GEFITINIB:Coronary artery disease:0.41), (GLIMEPIRIDE\ROSIGLITAZONE MALEATE:Coronary artery disease:1.34), (HERBALS\TURMERIC:Coronary artery disease:1.26), (HYDRAZINE SULFATE:Coronary artery disease:0), (HYDROCODONE BITARTRATE\IBUPROFEN:Coronary artery disease:0.47), (HYDROCORTISONE ACETATE:Coronary artery disease:0.56), (HYDROXYCHLOROQUINE DIPHOSPHATE:Coronary artery disease:0.7), (IDARUCIZUMAB:Coronary artery disease:1.81), (IMIGLUCERASE:Coronary artery disease:0.52), (IMMUNE GLOBULIN NOS:Coronary artery disease:0.06), (INDAPAMIDE\PERINDOPRIL:Coronary artery disease:2.79), (INSULIN PORK\INSULIN PURIFIED PORK:Coronary artery disease:0.32), (INTERFERON:Coronary artery disease:0.75), (INTERFERON ALFA-2A:Coronary artery disease:1.06), (LABETALOL HYDROCHLORIDE:Coronary artery disease:1.53), (LACTOBACILLUS RHAMNOSUS:Coronary artery disease:0.61), (LANREOTIDE:Coronary artery disease:0.13), (LANREOTIDE ACETATE:Coronary artery disease:0.19), (LAPATINIB DITOSYLATE:Coronary artery disease:0.62), (LAPINE T-LYMPHOCYTE IMMUNE GLOBULIN:Coronary artery disease:0.04), (LENVATINIB:Coronary artery disease:0.38), (LEUCOVORIN CALCIUM:Coronary artery disease:1.44), (LEVOCETIRIZINE DIHYDROCHLORIDE:Coronary artery disease:0.54), (LEVOMEFOLATE CALCIUM:Coronary artery disease:0.1), (LIOTHYRONINE SODIUM:Coronary artery disease:0.83), (LISDEXAMFETAMINE DIMESYLATE:Coronary artery disease:0.69), (LUTETIUM LU-177:Coronary artery disease:0.13), (MEPOLIZUMAB:Coronary artery disease:0.88), (METFORMIN HYDROCHLORIDE\ROSIGLITAZONE MALEATE:Coronary artery disease:1.66), (METFORMIN PAMOATE:Coronary artery disease:0.33), (METHENOLONE ENANTHATE:Coronary artery disease:0.13), (METRELEPTIN:Coronary artery disease:0.1), (MOSAPRIDE CITRATE:Coronary artery disease:0.11), (NIRAPARIB:Coronary artery disease:0.83), (NOREPINEPHRINE BITARTRATE:Coronary artery disease:1.44), (ORPHENADRINE:Coronary artery disease:0.37), (OXYBUTYNIN\OXYBUTYNIN CHLORIDE:Coronary artery disease:0.37), (PANTOPRAZOLE MAGNESIUM:Coronary artery disease:2.34), (PENICILLIN G:Coronary artery disease:0.58), (PENICILLIN V:Coronary artery disease:0.67), (PENICILLIN V POTASSIUM:Coronary artery disease:0.04), (PENTAMIDINE ISETHIONATE:Coronary artery disease:0.31), (PENTOSAN POLYSULFATE SODIUM:Coronary artery disease:0.39), (PENTOSTATIN:Coronary artery disease:0.16), (PHENAZOPYRIDINE:Coronary artery disease:0.06), (PHENAZOPYRIDINE HYDROCHLORIDE:Coronary artery disease:0.16), (PHLOROGLUCINOL\1,3,5-TRIMETHOXYBENZENE:Coronary artery disease:0.08), (PILSICAINIDE HYDROCHLORIDE:Coronary artery disease:0.14), (PREDNISOLONE ACETATE:Coronary artery disease:1.84), (PROGESTERONE:Coronary artery disease:0.08), (RADIATION THERAPY:Coronary artery disease:1.51), (RETEPLASE:Coronary artery disease:0.32), (RETINOL:Coronary artery disease:0.57), (RILPIVIRINE HYDROCHLORIDE:Coronary artery disease:0.03), (RIOCIGUAT:Coronary artery disease:0.98), (ROSIGLITAZONE MALEATE:Coronary artery disease:1.95), (ROTIGOTINE:Coronary artery disease:0.17), (SCOPOLAMINE HYDROBROMIDE:Coronary artery disease:0.05), (SIPULEUCEL-T:Coronary artery disease:0.4), (SODIUM CITRATE:Coronary artery disease:0.51), (SODIUM PHOSPHATE, DIBASIC\SODIUM PHOSPHATE, MONOBASIC:Coronary artery disease:1.18), (SOFOSBUVIR\VELPATASVIR:Coronary artery disease:0.37), (SOLIFENACIN:Coronary artery disease:0.22), (SORBITOL:Coronary artery disease:0.13), (STREPTOKINASE:Coronary artery disease:0.1), (SULFAMETHOXAZOLE:Coronary artery disease:1.54), (TEDUGLUTIDE\WATER:Coronary artery disease:0.29), (TELOTRISTAT ETHYL:Coronary artery disease:0.25), (TEMOZOLOMIDE:Coronary artery disease:0.66), (TEMSIROLIMUS:Coronary artery disease:0.32), (TENECTEPLASE:Coronary artery disease:1.11), (TERBUTALINE:Coronary artery disease:0.93), (TESTOSTERONE UNDECANOATE:Coronary artery disease:0.74), (TIGECYCLINE:Coronary artery disease:0.28), (TIMOLOL\TRAVOPROST:Coronary artery disease:0), (TOBRAMYCIN:Coronary artery disease:0.72), (TRIHEXYPHENIDYL:Coronary artery disease:0.18), (TRIMIPRAMINE MALEATE:Coronary artery disease:0.73), (UROKINASE:Coronary artery disease:0.14), (VASOPRESSIN:Coronary artery disease:0.79), (VINCRISTINE SULFATE:Coronary artery disease:0.7), (VINORELBINE\VINORELBINE TARTRATE:Coronary artery disease:0.38), (ZILEUTON:Coronary artery disease:0.35), (ZOLMITRIPTAN:Coronary artery disease:0.29), (WARFARIN:Coronary artery insufficiency:0.04), (DILTIAZEM HYDROCHLORIDE:Coronary artery occlusion:0.14), (IBUPROFEN:Coronary artery occlusion:0.05), (NAPROXEN:Coronary artery occlusion:0.16), (VERAPAMIL HYDROCHLORIDE:Coronary artery occlusion:0.08), (WARFARIN:Coronary artery occlusion:0.44), (ZONISAMIDE:Cortical dysplasia:0.48), (.ALPHA.-TOCOPHEROL\VITAMINS:Cough:0.2), (ABOBOTULINUMTOXINA:Cough:0.25), (ACETA MINOPHEN\CAFFEINE:Cough:0.88), (AJMALINE:Cough:0.05), (ALBIGLUTIDE:Cough:0.34), (ALBUTEROL\GUAIFENESIN:Cough:0.91), (AMEZINIUM METILSULFATE:Cough:0.47), (AMILORIDE HYDROCHLORIDE\FUROSEMIDE:Cough:0.07), (AMINO ACIDS\CARBOHYDRATES\ELECTROLYTES NOS\VITAMINS:Cough:0.41), (AMINO ACIDS\CARBOHYDRATES\MINERALS\SOYBEAN OIL\VITAMINS:Cough:0.14), (AMISULPRIDE:Cough:1.21), (AMLODIPINE BESYLATE\HYDROCHLOROTHIAZIDE\VALSARTAN:Cough:1.31), (AMOXAPINE:Cough: 0.47), (AMOXICILLIN SODIUM:Cough:0.04), (AMPICILLIN SODIUM:Cough:0.09), (ANAGRELIDE HYDROCHLORIDE:Cough:0.47), (ANIDULAFUNGIN:Cough:0.45), (ANTIHISTAMINES NOS:Cough:1.33), (ARIPIPRAZOLE LAUROXIL:Cough:0.14), (ASCORBIC ACID/CHROMIUM/SELENIUM/VANADYL SULFATE:Cough:0.15), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Cough:0.37), (ASIAN GINSENG:Cough:0.61), (ASPIRIN\CAFFEINE\SALICYLAMIDE:Cough:0.19), (AVANAFIL:Cough: 0.43), (AZITHROMYCIN OR METHOTREXATE SODIUM:Cough:0.01), (BECLOMETHASONE DIPROPIONATE\FORMOTEROL:Cough:0.42), (BELATACEPT:Cough:0.98), (BEPOTASTIN E BESILATE:Cough:0.17), (BERAPROST:Cough:0.19), (BETAMIPRON\PANIPENEM:Cough:0.24), (BEZAFIBRATE:Cough:0.27), (BIFIDOBACTERIUM LONGUM\LACTOBACILLUS ACIDOPHILUS:Cough:0), (BIFIDOBACTERIUM LONGUM\LACTOBACILLUS ACIDOPHILUS\LACTOBACILLUS RHAMNOSUS:Cough:0.09), (BILASTINE:Cough:0.83), (BIMATOPROST\TIMOLOL:Cough:0.54), (BLESELUMAB:Cough:0.17), (BLINATUMOMAB:Cough:1.68), (BRINZOLAMIDE\TIM OLOL MALEATE:Cough:0.13), (BUCILLAMINE:Cough:0.58), (BUPIVACAINE HYDROCHLORIDE:Cough:0.19), (BUPRENORPHINE\NALOXONE:Cough:0.01), (CABOZA NTINIB:Cough:0.2), (CAFFEINE:Cough:0), (CALCIUM CARBONATE\ERGOCALCIFEROL:Cough:0.44), (CALCIUM CARBONATE\ETIDRONATE DISODIUM:Cough:0.01), (CALCIUM CHLORIDE\\ICODEXTRIN\MAGNESIUM CHLORIDE\SODIUM CHLORIDE\SODIUM LACTATE:Cough:0.13), (CALCIUM PANTOTHENATE\CYANOCOBALAMIN\NIACINAMIDE\PYRIDOXINE HYDROCHLORIDE\RIBOFLAVIN\THIAMINE MONONITRATE:Cough:0.66), (CAMPHOR\DIPHENHYDRAMINE\ZINC OXIDE:Cough:0.28), (CARPERITIDE:Cough:0.27), (CARTEOLOL:Cough:0.15), (CARTEOLO L HYDROCHLORIDE:Cough:0.03), (CEFCAPENE PIVOXIL HYDROCHLORIDE HYDRATE:Cough:0.49), (CEFOPERAZONE:Cough:0.25), (CEFOXITIN\CEFOXITIN SODIUM:Cough:0.5), (CEFTAZIDIME SODIUM:Cough:0.37), (CELLULASE\DIASTASE\LIPASE:Cough:0.03), (CEPHARANTHINE: Cough:0.1), (CERTOPARIN SODIUM:Cough:0.48), (CHLORDIAZEPOXIDE:Cough:0.35), (CHOLESTEROL:Cough:0.43), (CHONDROITIN SULFATE (CHICKEN):Cough:0.81), (CICLOPIROX OLAMINE:Cough:0.24), (CITRIC ACID MONOHYDRATE\POTASSIUM CITRATE:Cough:0.06), (CLADRIBINE:Cough:0.02), (CLIKSTAR:Cough:0.17), (CLODRONA TE DISODIUM:Cough:0.08), (CLOFARABINE:Cough:0.32), (CLOMIPRAMINE HYDROCHLORIDE:Cough:1.35), (CLOXAZOLAM:Cough:0.34), (COCONUT OIL:Cough:0.02), (CROTALIDAE POLYVALENT IMMUNE FAB (OVINE):Cough:0.03), (CYAMEMAZINE:Cough:0.38), (CYPROTERONE:Cough:0.12), (DABI GATRAN:Cough:1.4), (DACLIZUMAB:Cough:2.39), (DANTROLENE SODIUM:Cough:0.91), (DAPAGLIFLOZIN PROPANEDIOL\METFORMIN HYDROCHLORIDE:Cough:0.37), (DEFERIPRONE:Cough:0.79), (DEGARELIX:Cough:0.54), (DESFLURANE:Cough:0.79), (DESMOPRESSIN:Cough:1.34), (DESVENLAFAXINE:Cough:0.12), (DEXPANTHENOL\NIACINAMIDE\PYRIDOXINE HYDROCHLORIDE\RIBOFLAVIN 5′-PHOSPHATE SODIUM\THIAMINE HYDROCHLORIDE:Cough:0.22), (DEXTROAMPHETAMINE:Cough:0.54), (DEXTROAMPH ETAMINE SULFATE:Cough:1.32), (DIAZOXIDE:Cough:0.08), (DIFLUNISAL:Cough:0.36), (DILAZEP:Cough: 0.39), (DIPHENIDOL HYDROCHLORIDE:Cough:0.35), (DIPYRONE\PROMETHAZINE HYDROCHLORIDE:Cough:0.46), (DOBUTAMINE HYDROCHLORIDE:Cough:0.79), (DORIPENEM:Cough:0.34), (ECALLANTIDE:Cough:0.13), (EDARAVONE:Cough:0.12), (EDOXABAN TOSILATE MONOHYDRATE:Cough:0.01), (EMTRICITABINE\RILPIVIRINE HYDROCHLORIDE\TENOFOVIR DISOPROXIL FUMARATE:Cough:0.5), (EMTRICITABINE\RILPIVIRINE\TENOFOVIR DISOPROXIL FUMARATE:Cough:0.29), (ENALAPRIL MALEATE\HYDROCHLOROTHIAZIDE:Cough:0.59), (ENALAPRIL MALEATE\LERCANIDIPINE HYDROCHLORIDE:Cough:0.3), (EPHEDRINE HYDROCHLORIDE:Cough:0.06), (EPHEDRINE HYDROCHLORIDE\GUAIFENESIN:Cough:0.57), (EPIRUBICIN:Cough:1.15), (EPIRUBICIN HYDROCHLORIDE:Cough:0.05), (ERYTHROMYCIN LACTOBIONATE:Cough:0.39), (ESMOLOL HYDROCHLORIDE:Cough:0.35), (ESTROGENS, ESTERIFIED\METHYLTESTOSTERONE:Cough:0.29), (ESTROPIPATE:Cough:0.02), (ETHI NYL ESTRADIOL\NORETHINDRONE:Cough:0.33), (ETHINYL ESTRADIOL\NORGESTREL:Cough:1.05), (ETHYL ICOSAPENTATE\ICOSAPENT:Cough:0.14), (ETRETINATE:Cough:1.87), (EUCALYPTOL\M ENTHOL\METHYL SALICYLATE\THYMOL:Cough:0.11), (FELBAMATE:Cough:0.1), (FERRIC HYDROXIDE:Cough:0.05), (FERRIC POLYSACCHARIDE COMPLEX:Cough:0.05), (FERROUS BISGLYCINATE:Cough:0.16), (FERROUS SULFATE\FOLIC ACID:Cough:0.6), (FERROUS SULFATE\GLYCINE:Cough:0.95), (FIBRINOGEN HUMAN:Cough:0.09), (FILGRASTIM-SNDZ:Cough:0.17), (FISH OIL\TOCOPHEROL:Cough:0.21), (FLAVIN ADENINE DINUCLEOTIDE:Cough:0.41), (FLUCYTOSINE:Cough:0.66), (FLUNARIZINE HYDROCHLORIDE:Cough:0.4), (FLUOXETINE HYDROCHLORIDE\OLANZAPINE:Cough:0.05), (FLUTAMIDE:Cough:0.34), (FOSFLUCON AZOLE:Cough:0.21), (FOSFOMYCIN:Cough:1.12), (FOSINOPRIL SODIUM:Cough:0.33), (FROVATRIPTAN SUCCINATE:Cough:0.54), (GADOTERATE MEGLUMINE:Cough:0.17), (GLUCONATE:Cough:0.23), (GLYCOPYRROLATE\INDACATE ROL MALEATE:Cough:1.4), (GUAIFENESIN\PHENYLEPHRINE HYDROCHLORIDE:Cough:0.22), (GUSPERIMUS TRIHYDROCHLORIDE:Cough:0.29), (HIGH DENSITY POLYETHYLENE:Cough:0.03), (HUMAN C1-ESTERASE INHIBITOR:Cough:0.01), (HYDROCODONE BITARTRATE\IBUPROFEN:Cough:1.09), (HYDROCORTISONE ACETATE:Cough:1.79), (HYOSCYAMINE SULFATE\METHENAMINE\METHYLENE BLUE\PHENYL SALICYLATE\SODIUM PHOSPHATE, MONOBASIC, MONOHYDRATE:Cough:0.34), (IBRITUMOMAB TIUXETAN:Cough:0.69), (ILOPROST:Cough:2.17), (INDACATEROL MALEATE:Cough:0.05), (INDAPAMIDE\PERINDOPRIL:Cough:2.61), (INFLIXIMAB-DYYB:Cough:0.88), (INTERFERON ALFA-2A:Cough:1.83), (INTERFERON ALFA-2A OR INTERFERON ALFA-2B:Cough:0.26), (INTERFERON ALFA-2B: Cough:1.67), (INTERFERON GAMMA-B:Cough:1.76), (IRON\POLYSACCHARIDES:Cough:0.68), (ISOFLURANE:Cough:0.17), (ISO NIAZID\RIFAMPIN:Cough:0.26), (KETAMINE:Cough:0.27), (LANREOTIDE:Cough:1.16), (L ATANOPROST\TIMOLOL:Cough:0.09), (LEUCOGEN:Cough:0.7), (LEVOTHYROXINE SODIUM\POTASSIUM IODIDE:Cough:0.79), (LOMUSTINE:Cough:0.37), (LULICONAZOLE:Cough:0.09), (LUMACA FTOR:Cough:1.33), (LYSOZYME HYDROCHLORIDE:Cough:0.17), (MAGNESIUM ASPARTATE\POTASSIUM ASPARTATE:Cough:0.55), (MAXACALCITOL:Cough:0.64), (MELPERONE:Cough:0.2), (ME TFORMIN HYDROCHLORIDE\SAXAGLIPTIN:Cough:0.1), (METFORMIN HYDROCHLORIDE\SAXAGLIPTIN HYDROCHLORIDE:Cough:0.58), (METHENAMINE:Cough:0.02), (METHENAMINE HIPPURATE:Cough:2.41), (METHENAMINE MANDELATE:Cough:0.22), (METHENOLONE ENANTHATE:Cough:1.79), (METYRAPONE:Cough:0.12), (MEXILETINE:Cough:0.42), (MEX ILETINE HYDROCHLORIDE:Cough:0.63), (MIANSERIN:Cough:0.32), (MIGLUSTAT:Cough:0.52), (MI TOXANTRONE HYDROCHLORIDE:Cough:0.54), (MOEXIPRIL HYDROCHLORIDE:Cough:0.94), (NALOXONE:Cough:0.8), (NUSINERSEN:Cough:0.23), (0B ETICHOLIC ACID:Cough:0.05), (OCTREOTIDE:Cough:1.85), (OPIPRAMOL:Cough:0.58), (OXYBUTYNIN \OXYBUTYNIN CHLORIDE:Cough:0.81), (OXYMORPHONE:Cough:0.21), (PAPAYA:Cough:0.12), (PARATH YROID HORMONE:Cough:0.64), (PEGINTERFERON BETA-1A:Cough:1.53), (PEGZEREPOETIN ALFA:Cough:0.07), (PENICILLIN G:Cough:1.47), (PENICILLIN V:Cough:1.64), (PENICILLIN V BENZATHINE:Cough:0.1), (PENTOSTATIN:Cough:1.39), (PHENTERMINE HYDROCHLORIDE\TOPIRAMATE:Cough:0.04), (PINDOLOL:Cough:0.17), (POLYETHYLE NE GLYCOL 400:Cough:0.4), (POLYMYXIN B SULFATE:Cough:0.18), (POLYMYXIN B SULFATE\TRIMETHOPRIM SULFATE:Cough:0.57), (POLYVINYL ALCOHOL:Cough:0.19), (POLYVINYL ALCOHOL\POVIDONE:Cough:0.4), (PORFIMER SODIUM:Cough:0.61), (POTASSIUM CITRATE\TRISODIUM CITRATE DIHYDRATE:Cough:1), (PREDNISOLONE SODIUM SUCCINATE:Cough:0.68), (PROTRIPTYLINE HYDROCHLORIDE:Cough:0.84), (PYRIDOXAL:Cough:0.61), (PYRIDOXAL PHOSPHATE:Cough:0.27), (PYRIMETHAMINE:Cough:0.21), (QUINACRINE:Cough:0.76), (R ANIMUSTINE:Cough:0.21), (RHUBARB:Cough:0.23), (RILPIVIRINE HYDROCHLORIDE:Cough:0.57), (RUPATADINE FUMARATE:Cough:0.1), (SELEGILINE HYDROCHLORIDE:Cough:0.15), (SERRAPEPTASE:Cough:0.18), (SIBUTRAMINE HYDROCHLORIDE:Cough:0.47), (SILICON DIOXIDE:Cough:0.23), (SODIUM:Cough:0.08), (SODIUM CITRATE:Cough:0.75), (SODIUM GUALENATE MONOHYDRATE:Cough:0.18), (SOMATOSTATIN:Cough:0.02), (STREPTOMYCIN\STREPT OMYCIN SULFATE:Cough:0.27), (SUCCINYLCHOLINE CHLORIDE:Cough:0.17), (SUFENTANIL CITRATE:Cough:0.43), (SULBACTAM SODIUM:Cough:0.1), (SULPIRIDE:Cough:0.57), (SULTOPRIDE HYDROCHLORIDE:Cough:0.01), (SUPLATAST TOSILATE:Cough:1.13), (TABALUMAB:Cough:0.05), (TBO-FILGRASTIM:Cough:0.26), (TEDUGLUTIDE:Cough:0.37), (TEDUGLUTIDE\WATER:Cough: 1.35), (TELOTRISTAT ETHYL:Cough:1.03), (TEMOCAPRIL HYDROCHLORIDE:Cough:0.18), (TESTOSTERONE UNDECANOATE:Cough:1.63), (TETRABENAZINE:Cough:0.15), (THIOCTIC ACID:Cough:0.01), (THIOPENTAL SODIUM:Cough:0.23), (THROMBOMODULIN ALFA:Cough:0.54), (TIBOLONE:Cough:0.21), (TICLOPIDINE:Cough:0.32), (TIMOLOL MALEATE\TRAVOPROST:Cough:0.86), (TIMOLOL\TRAVOPROST:Cough:0.13), (TRABEC TEDIN:Cough:0.19), (TRIHEXYPHENIDYL:Cough:0.12), (TROSPIUM:Cough:0.02), (URAPID IL:Cough:1.18), (VINDESINE SULFATE:Cough:0.03), (WARFARIN POTASSIUM:Cough:1.06), (YEAST:Cough:1), (ZICONOTIDE ACETATE:Cough:0.06), (ZIV-AFLIBERCEPT:Cough:0.44), (PHENOBARBITAL:Craniocerebral injury:0.12), (IBRUTINIB:CREST syndrome:0.15), (UNSPECIFIED INGREDIENT:CREST syndrome:0.19), (ABIRATERONE ACETATE:Crohn's disease:0.03), (ABOBOTULINUMTOXINA:Crohn's disease:0.59), (ACETAMINOPHEN\CAFFEINE\CARISOPRODOL\DICLOFENAC:Crohn's disease:0.23), (ACETAMINOPHEN\PROPOXYPHENE NAPSYLATE:Crohn's disease:2.31), (ADENOSINE:Crohn's disease:0.22), (ADO-TRASTUZUMAB EMTANSINE:Crohn's disease:0.49), (AGALSIDASE BETA:Crohn's disease:1.59), (ALBENDAZOLE:Crohn's disease:0.02), (ALGLUCOSIDASE ALFA:Crohn's disease:0.23), (ALISKIREN:Crohn's disease:0.54), (ALISKIREN HEMIFUMARATE\HYDROCHLOROTHIAZIDE:Crohn's disease:0.54), (ALUMINUM HYDROXIDE\MAGNESIUM HYDROXIDE\SIMETHICONE:Crohn's disease:0.38), (AMILORIDE HYDROCHLORIDE\FUROSEMIDE:Crohn's disease:0.16), (AMINOPHYLLINE:Crohn's disease:0.05), (AMOXICILLIN\CLARITHROMYCIN\ESOMEPRAZOLE:Crohn's disease:1.28), (APPLE CIDER VINEGAR:Crohn's disease:0.29), (ARIPIPRAZOLE LAUROXIL:Crohn's disease:0.67), (ASCORBIC ACID/CHROMIUM/SELENIUM/VANADYL SULFATE:Crohn's disease:0.62), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Crohn's disease:0.33), (ASCORBIC ACID\FERROUS SULFATE\FOLIC ACID\PYRIDOXINE HYDROCHLORIDE\RIBOFLAVIN\THIAMINE MONONITRATE:Crohn's disease:0.38), (ASPIRIN DL-LYSINE:Crohn's disease:0.4), (ASPIRIN\CLOPIDOGREL:Crohn's disease:0.09), (ASPIRIN\MAGNESIUM OXIDE:Crohn's disease:0.66), (ATOVAQUONE\PROGUANIL HYDROCHLORIDE:Crohn's disease:0.15), (AVOCADO OIL\SOYBEAN OIL:Crohn's disease:0.2), (AXITINIB:Crohn's disease:0.69), (AZITHROMYCIN OR METHOTREXATE SODIUM:Crohn's disease:1.59), (BAMIFYLLINE HYDROCHLORIDE:Crohn's disease:0.5), (BAZEDOXIFENE ACETATE\ESTROGENS, CONJUGATED:Crohn's disease:0.45), (BELATACEPT:Crohn's disease:0.56), (BENDROFLUMETHIAZIDE:Crohn's disease:0.91), (BENSERAZIDE\LEVODOPA:Crohn's disease:0.26), (BETAMETHASONE ACETATE\BETAMETHASONE SODIUM PHOSPHATE:Crohn's disease:0.21), (BETAMETHASONE DIPROPIONATE\CALCIPOTRIENE HYDRATE:Crohn's disease:1.28), (BIFIDOBACTERIUM ANIMALIS LACTIS:Crohn's disease:0.29), (BIFIDOBACTERIUM LONGUM\LACTOBACILLUS ACIDOPHILUS:Crohn's disease:0.02), (BIMATOPROST\TIMOLOL:Crohn's disease:0.17), (BLINATUMOMAB:Crohn's disease:0.03), (BOCEPREVIR:Crohn's disease:0.33), (BORIC ACID:Crohn's disease:0.21), (BORON:Crohn's disease:0.01), (BRINZOLAMIDE\TIMOLOL MALEATE:Crohn's disease:0.2), (BROMPHENIRAMINE MALEATE\PSEUDOEPHEDRINE HYDROCHLORIDE:Crohn's disease:0.13), (BUPRENORPHINE\NALOXONE:Crohn's disease:0.16), (CABOZANTINIB S-MALATE:Crohn's disease:0), (CALCIUM CARBONATE\ERGOCALCIFEROL\RETINOL:Crohn's disease:1.08), (CALCIUM PANTOTHENATE\CYANOCOBALAMIN\NIACINAMIDE\PYRIDOXINE HYDROCHLORIDE\RIBOFLAVIN\THIAMINE HYDROCHLORIDE:Crohn's disease:0.08), (CAMPHOR\DIPHENHYDRAMINE\ZINC OXIDE:Crohn's disease:0.33), (CANAGLIFLOZIN\METFORMIN HYDROCHLORIDE:Crohn's disease:1.51), (CARBAMIDE PEROXIDE:Crohn's disease:0.16), (CARBIDOPA\ENTACAPONE\LEVODOPA:Crohn's disease:0.15), (CEFEPIME\CEFEPIME HYDROCHLORIDE:Crohn's disease:0.83), (CEFOTAXIME SODIUM:Crohn's disease:1.78), (CEFTAZIDIME SODIUM:Crohn's disease:0.03), (CEFUROXIME SODIUM:Crohn's disease:0.4), (CELIPROLOL:Crohn's disease:0.21), (CERIVASTATIN SODIUM:Crohn's disease:0.84), (CERTOPARIN SODIUM:Crohn's disease:0.41), (CEVIMELINE:Crohn's disease:0.13), (CHLORAMBUCIL:Crohn's disease:0.39), (CHLORMADINONE ACETATE:Crohn's disease:0.25), (CHLORMADINONE ACETATE\ETHINYL ESTRADIOL:Crohn's disease:0.44), (CHLOROTHIAZIDE:Crohn's disease:0.85), (CHONDROITIN SULFATE (CHICKEN):Crohn's disease:0.73), (CICLOPIROX OLAMINE:Crohn's disease:0.49), (CLIKSTAR:Crohn's disease:0.52), (CLOCORTOLONE PIVALATE:Crohn's disease:0.63), (CODEINE PHOSPHATE\DICLOFENAC SODIUM:Crohn's disease:0.27), (COLCHICINE\DICYCLOMINE HYDROCHLORIDE:Crohn's disease:0.14), (COLISTIMETHATE SODIUM:Crohn's disease:0.31), (CYANOCOBALAMIN\DICLOFENAC SODIUM\PYRIDOXINE HYDROCHLORIDE\THIAMINE MONONITRATE:Crohn's disease:0.19), (CYANOCOBALAMIN\PYRIDOXINE HYDROCHLORIDE\THIAMINE DISULFIDE:Crohn's disease:0.41), (DABIGATRAN:Crohn's disease:0.83), (DACLIZUMAB:Crohn's disease:2.28), (DANAZOL:Crohn's disease:1.45), (DANTROLENE SODIUM:Crohn's disease:0.83), (DAPTOMYCIN:Crohn's disease:0.4), (DASABUVIR\OMBITASVIR\PARITAPREVIR\RITONAVIR:Crohn's disease:0.98), (DEXAMETHASONE ACETATE:Crohn's disease:0.78), (DEXAMETHASONE SODIUM PHOSPHATE:Crohn's disease:1.11), (DEXTROMETHORPHAN\DEXTROMETHORPHAN HYDROBROMIDE\GUAIFENESIN:Crohn's disease:0.04), (DIGITOXIN:Crohn's disease:0.29), (DIOSMIN\HESPERIDIN:Crohn's disease:0.2), (DIPHTHERIA AND TETANUS TOXOIDS AND ACELLULAR PERTUSSIS VACCINE ADSORBED:Crohn's disease:0.3), (DOLUTEGRAVIR SODIUM:Crohn's disease:0.05), (DORZOLAMIDE HYDROCHLORIDE:Crohn's disease:1.4), (DOXYCYCLINE HYDROCHLORIDE:Crohn's disease:0.26), (DROSPIRENONE:Crohn's disease:0.25), (DROSPIRENONE\ESTRADIOL:Crohn's disease:0.07), (DYDROGESTERONE:Crohn's disease:0.42), (ECALLANTIDE:Crohn's disease:0.13), (EDOXABAN TOSYLATE:Crohn's disease:1.37), (EFINACONAZOLE:Crohn's disease:0.6), (ELTROMBOPAG OLAMINE:Crohn's disease:0.43), (ESTAZOLAM:Crohn's disease:0.05), (ESTRADIOL BENZOATE:Crohn's disease:0.65), (ESTRADIOL CYPIONATE:Crohn's disease:0.37), (ESTRADIOL HEMIHYDRATE:Crohn's disease:0.15), (ESTRADIOL\LEVONORGESTREL:Crohn's disease:0.22), (ESTROGENS\METHYLTESTOSTERONE:Crohn's disease:0.61), (ETHACRYNIC ACID:Crohn's disease:0.96), (ETHINYL ESTRADIOL\ETHYNODIOL:Crohn's disease:1.21), (ETRETINATE:Crohn's disease:0.28), (FELBAMATE:Crohn's disease:0.11), (FINGOLIMOD:Crohn's disease:0.06), (FLAVIN ADENINE DINUCLEOTIDE:Crohn's disease:0.01), (FLAVOXATE HYDROCHLORIDE:Crohn's disease:0.04), (FLUOXETINE HYDROCHLORIDE\OLANZAPINE:Crohn's disease:0.39), (FONDAPARINUX:Crohn's disease:0.8), (FOSINOPRIL SODIUM:Crohn's disease:0.64), (FOSPHENYTOIN\FOSPHENYTOIN SODIUM:Crohn's disease:0.22), (GADOLINIUM:Crohn's disease:0.38), (GLYCOPYRROLATE\INDACATEROL MALEATE:Crohn's disease:0.79), (GREEN TEA LEAF:Crohn's disease:0.15), (HALOPERIDOL DECANOATE:Crohn's disease:0.05), (HORSE CHESTNUT:Crohn's disease:0.85), (HYDROQUINONE:Crohn's disease:0.18), (IDELALISIB:Crohn's disease:0.31), (ILOPROST:Crohn's disease:0.09), (IMIGLUCERASE:Crohn's disease:1.01), (IMIPENEM:Crohn's disease:0.74), (INDACATEROL:Crohn's disease:1.06), (INOTUZUMAB OZOGAMICIN:Crohn's disease:0.83), (INSULIN PORK\INSULIN PURIFIED PORK:Crohn's disease:0.39), (INTERFERON:Crohn's disease:1.8), (INTERFERON ALFA-2A:Crohn's disease:0.66), (INTERFERON GAMMA-1B:Crohn's disease:1.26), (ITRACONAZOLE\SODIUM CHLORIDE:Crohn's disease:0.56), (IVACAFTOR:Crohn's disease:0.16), (IXAZOMIB:Crohn's disease:0.72), (LANREOTIDE:Crohn's disease:0.61), (LAPATINIB DITOSYLATE:Crohn's disease:0.24), (LERCANIDIPINE HYDROCHLORIDE:Crohn's disease:1.99), (LEVALBUTEROL TARTRATE:Crohn's disease:0.75), (LEVOLEUCOVORIN CALCIUM:Crohn's disease:0.6), (LOMITAPIDE MESYLATE:Crohn's disease:1.32), (LUTETIUM LU-177:Crohn's disease:0.64), (MAGNESIUM ASPARTATE HYDROCHLORIDE:Crohn's disease:0.97), (MAGNESIUM ASPARTATE\POTASSIUM ASPARTATE:Crohn's disease:0.01), (MECLOZINE DIHYDROCHLORIDE:Crohn's disease:0.02), (MELPERONE:Crohn's disease:1.03), (MEPOLIZUMAB:Crohn's disease:1.08), (METFORMIN HYDROCHLORIDE\VILDAGLIPTIN:Crohn's disease:0.71), (METHENAMINE MANDELATE:Crohn's disease:0.63), (MICAFUNGIN:Crohn's disease:0.21), (MIGLUSTAT:Crohn's disease:0.25), (MILNACIPRAN:Crohn's disease:0.06), (MOLSIDOMINE:Crohn's disease:0.01), (NEFAZODONE:Crohn's disease:0.32), (NEFAZODONE HYDROCHLORIDE:Crohn's disease:0.38), (NICARDIPINE HYDROCHLORIDE:Crohn's disease:0.8), (NIMESULIDE:Crohn's disease:0.69), (NIRAPARIB:Crohn's disease:0.29), (NISOLDIPINE:Crohn's disease:0.51), (NOREPINEPHRINE:Crohn's disease:0.61), (OLODATEROL HYDROCHLORIDE:Crohn's disease:0.06), (OMEGA-6 FATTY ACIDS:Crohn's disease:0.36), (OXYBUTYNIN\OXYBUTYNIN CHLORIDE:Crohn's disease:1.02), (PALIPERIDONE PALMITATE:Crohn's disease:0.7), (PALONOSETRON HYDROCHLORIDE:Crohn's disease:0.36), (PARATHYROID HORMONE:Crohn's disease:0.33), (PASIREOTIDE:Crohn's disease:0.67), (PEGINTERFERON ALFA-2A OR PEGINTERFERON ALFA-2B:Crohn's disease:0.17), (PEGINTERFERON ALFA-2A OR PEGINTERFERON ALFA-2B OR PEGINTERFERON BETA-1A OR PEGINTERFERON LAMBDA-1A:Crohn's disease:0.01), (PEMETREXED DISODIUM:Crohn's disease:0.53), (PENICILLAMINE:Crohn's disease:0.32), (PENTAMIDINE:Crohn's disease:0.16), (PENTAMIDINE ISETHIONATE:Crohn's disease:0.54), (PEPSIN:Crohn's disease:0.04), (PHENELZINE SULFATE:Crohn's disease:0.18), (PONATINIB:Crohn's disease:1.5), (POTASSIUM IODIDE:Crohn's disease:0.02), (POVIDONE:Crohn's disease:0.91), (PRAMLINTIDE ACETATE:Crohn's disease:0.42), (PROPATYL NITRATE:Crohn's disease:0.16), (PROPOXYPHENE:Crohn's disease:0.1), (PSORALEN:Crohn's disease:0.18), (PYRIDOSTIGMINE:Crohn's disease:0.23), (PYRIMETHAMINE:Crohn's disease:0.74), (QUINACRINE:Crohn's disease:1.72), (QUINIDINE GLUCONATE:Crohn's disease:0.08), (RADIATION THERAPY:Crohn's disease:0.31), (REGORAFENIB:Crohn's disease:0.7), (RILMENIDINE PHOSPHATE:Crohn's disease:0.69), (RILPIVIRINE HYDROCHLORIDE:Crohn's disease:0.62), (RISEDRONIC ACID:Crohn's disease:0.06), (RIVASTIGMINE:Crohn's disease:1.44), (ROFECOXIB:Crohn's disease:0.87), (SAPROPTERIN DIHYDROCHLORIDE:Crohn's disease:0.5), (SELEGILINE HYDROCHLORIDE:Crohn's disease:0.05), (SEVELAMER HYDROCHLORIDE:Crohn's disease:0.86), (SIBUTRAMINE HYDROCHLORIDE:Crohn's disease:1.22), (SILICON DIOXIDE:Crohn's disease:0.71), (SIPULEUCEL-T:Crohn's disease:0.05), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Crohn's disease:0.08), (SUFENTANIL CITRATE:Crohn's disease:0.48), (TASIMELTEON:Crohn's disease:0.53), (TEGASEROD:Crohn's disease:0.25), (TELOTRISTAT ETHYL:Crohn's disease:0.35), (TESAMORELIN:Crohn's disease:0.41), (TESTOSTERONE UNDECANOATE:Crohn's disease:1.8), (TIAGABINE HYDROCHLORIDE:Crohn's disease:0.17), (TRIPTORELIN PAMOATE:Crohn's disease:0.33), (UMECLIDINIUM BROMIDE:Crohn's disease:0.94), (VALDECOXIB:Crohn's disease:0.9), (VILDAGLIPTIN:Crohn's disease:0.41), (VINORELBINE TARTRATE:Crohn's disease:0.07), (ZICONOTIDE ACETATE:Crohn's disease:1), (ZINC ACETATE:Crohn's disease:0.31), (ZIPRASIDONE HYDROCHLORIDE:Crohn's disease:0.28), (ZIV-AFLIBERCEPT:Crohn's disease:0.53), (ACETAMINOPHEN\OXYCODONE\OXYCODONE HYDROCHLORIDE:Cushing's syndrome:0.01), (AMLODIPINE BESYLATE\OLMESARTAN MEDOXOMIL:Cushing's syndrome:0.02), (AMOXICILLIN:Cushing's syndrome:0.48), (BISOPROLOL FUMARATE:Cushing's syndrome:1.11), (CALCIUM CITRATE:Cushing's syndrome:0.31), (CANDESARTAN CILEXETIL:Cushing's syndrome:0.77), (CAPTOPRIL:Cushing's syndrome:0.09), (CHOLESTYRAMINE:Cushing's syndrome:1.2), (CLOTRIMAZOLE:Cushing's syndrome:0.22), (CORTISONE\HYDROCORTISONE:Cushing's syndrome:0.15), (DALFAMPRIDINE:Cushing's syndrome:0.41), (DASATINIB:Cushing's syndrome:0.29), (DOCUSATE CALCIUM:Cushing's syndrome:0.24), (DOFETILIDE:Cushing's syndrome:0.44), (DRONABINOL:Cushing's syndrome:0.13), (ELETRIPTAN HYDROBROMIDE:Cushing's syndrome:0.66), (EMPAGLIFLOZIN\LINAGLIPTIN:Cushing's syndrome:0.74), (ERYTHROPOIETIN:Cushing's syndrome:0.15), (ETHAMBUTOL HYDROCHLORIDE:Cushing's syndrome:0.92), (EVOLOCUMAB:Cushing's syndrome:1.46), (FLUTICASONE:Cushing's syndrome:0.26), (HYDROCHLOROTHIAZIDE\TELMISARTAN:Cushing's syndrome:0.1), (HYDROMORPHONE:Cushing's syndrome:0.01), (HYDROXYZINE PAMOATE:Cushing's syndrome:0.77), (HYOSCYAMINE:Cushing's syndrome:0.59), (IBRUTINIB:Cushing's syndrome:1), (ILOPROST:Cushing's syndrome:0.12), (INDOMETHACIN:Cushing's syndrome:0.4), (LEFLUNOMIDE:Cushing's syndrome:0.1), (LENALIDOMIDE:Cushing's syndrome:0.07), (LEVALBUTEROL HYDROCHLORIDE:Cushing's syndrome:0.31), (LIDOCAINE HYDROCHLORIDE:Cushing's syndrome:0.01), (LOMITAPIDE MESYLATE:Cushing's syndrome:0.1), (METHENAMINE HIPPURATE:Cushing's syndrome:0.15), (METHYLPHENIDATE HYDROCHLORIDE:Cushing's syndrome:0.37), (METHYLPREDNISOLONE SODIUM SUCCINATE:Cushing's syndrome:0.71), (METRONIDAZOLE\METRONIDAZOLE HYDROCHLORIDE:Cushing's syndrome:0.53), (MIDODRINE:Cushing's syndrome:0.08), (MOXIFLOXACIN HYDROCHLORIDE:Cushing's syndrome:0.37), (NALOXONE HYDROCHLORIDE\OXYCODONE HYDROCHLORIDE:Cushing's syndrome:0.18), (NITROFURANTOIN:Cushing's syndrome:0.36), (OLANZAPINE:Cushing's syndrome:0.68), (PANTOPRAZOLE MAGNESIUM:Cushing's syndrome:0.34), (PEGVISOMANT:Cushing's syndrome:0.17), (PENICILLIN:Cushing's syndrome:0.06), (PERINDOPRIL:Cushing's syndrome:0.01), (PHENOBARBITAL:Cushing's syndrome:0.31), (POLYETHYLENE GLYCOLS:Cushing's syndrome:0.62), (PYRIDOXINE HYDROCHLORIDE:Cushing's syndrome:0.06), (RALOXIFENE HYDROCHLORIDE:Cushing's syndrome:0.3), (SACUBITRIL\VALSARTAN:Cushing's syndrome:0.21), (SODIUM BICARBONATE:Cushing's syndrome:0.13), (SODIUM OXYBATE:Cushing's syndrome:0.9), (TERIFLUNOMIDE:Cushing's syndrome:0.42), (TETRACYCLINE:Cushing's syndrome:0.42), (UMECLIDINIUM BROMIDE\VILANTEROL TRIFENATATE:Cushing's syndrome:0.15), (ZINC:Cushing's syndrome:0.19), ((UNSPECIFIED INGREDIENT:Cutaneous lupus erythematosus:0.3), (PYRAZINAMIDE:Cutaneous sarcoidosis:0.57), (FOLIC ACID:Cyst:0.6), (ACETAMINOPHEN\DIPHENHYDRAMINE:Cystic fibrosis:0.04), (ACETAMINOPHEN\OXYCODONE\OXYCODONE HYDROCHLORIDE:Cystic fibrosis:0.39), (ACETAMINOPHEN\PROPOXYPHENE HYDROCHLORIDE:Cystic fibrosis:0.34), (ALBUTEROL\GUAIFENESIN:Cystic fibrosis:0.08), (ALCOHOL:Cystic fibrosis:0.29), (ALGLUCOSIDASE ALFA:Cystic fibrosis:0.09), (AMIODARONE:Cystic fibrosis:0.64), (AMIODARONE HYDROCHLORIDE:Cystic fibrosis:0.14), (AMISULPRIDE:Cystic fibrosis:0.05), (AMLODIPINE\VALSARTAN:Cystic fibrosis:0.03), (ANASTROZOLE:Cystic fibrosis:0.74),(ARMODAFINIL:Cystic fibrosis:1.22), (BENAZEPRIL HYDROCHLORIDE:Cystic fibrosis:0.35), (BENAZEPRIL\BENAZEPRIL HYDROCHLORIDE:Cystic fibrosis:0.6), (BIFIDOBACTERIUM LONGUM SUBSP. INFANTIS:Cystic fibrosis:0.11), (BIMATOPROST:Cystic fibrosis:0.01), (BISOPROLOL FUMARATE:Cystic fibrosis:0.22), (BUPRENORPHINE HYDROCHLORIDE:Cystic fibrosis:0.46), (CALCITONIN SALMON:Cystic fibrosis:0.3), (CANAKINUMAB:Cystic fibrosis:0.73), (CANDESARTAN:Cystic fibrosis:0.33), (CANDESARTAN CILEXETIL:Cystic fibrosis:0.38), (CANDESARTAN CILEXETIL\HYDROCHLOROTHIAZIDE:Cystic fibrosis:0.45), (CARFILZOMIB:Cystic fibrosis:0.37), (CARVEDILOL PHOSPHATE:Cystic fibrosis:0.74), (CEFAZOLIN SODIUM:Cystic fibrosis:0.11), (CEFPROZIL:Cystic fibrosis:0.47), (CEFTRIAXONE SODIUM:Cystic fibrosis:1.44), (CERTOLIZUMAB PEGOL:Cystic fibrosis:1.26), (CETIRIZINE HYDROCHLORIDE\PSEUDOEPHEDRINE HYDROCHLORIDE:Cystic fibrosis:0.11), (CIMETIDINE:Cystic fibrosis:0.47), (CINACALCET HYDROCHLORIDE:Cystic fibrosis:0.12), (COD LIVER OIL:Cystic fibrosis:0.23), (CODEINE PHOSPHATE\GUAIFENESIN:Cystic fibrosis:0.02), (COLESEVELAM HYDROCHLORIDE:Cystic fibrosis:0.36), (CORTICOTROPIN:Cystic fibrosis:0.15), (DALTEPARIN:Cystic fibrosis:0.32), (DAPTOMYCIN:Cystic fibrosis:0.02), (DASATINIB:Cystic fibrosis:0.57), (DIMENHYDRINATE:Cystic fibrosis:0.52), (DIMETHICONE\SILICON DIOXIDE\SIMETHICONE:Cystic fibrosis:0.32), (DOFETILIDE:Cystic fibrosis:0.12), (ECONAZOLE:Cystic fibrosis:0.23), (ELETRIPTAN HYDROBROMIDE:Cystic fibrosis:1.37), (ENALAPRIL MALEATE:Cystic fibrosis:1.05), (ESOMEPRAZOLE MAGNESIUM\NAPROXEN:Cystic fibrosis:0.22), (ESTROGENS, CONJUGATED\MEDROXYPROGESTERONE ACETATE:Cystic fibrosis:0.49), (EVOLOCUMAB:Cystic fibrosis:0.5), (FESOTERODINE FUMARATE:Cystic fibrosis:0.4), (FLECAINIDE:Cystic fibrosis:0.89), (FLUCYTOSINE:Cystic fibrosis:0.11), (FLUOCINONIDE:Cystic fibrosis:0.17), (FLUVOXAMINE MALEATE:Cystic fibrosis:0.01), (GLICLAZIDE:Cystic fibrosis:0.21), (HERBALS\TURMERIC:Cystic fibrosis:0.02), (HYDROCHLOROTHIAZIDE\IRBESARTAN:Cystic fibrosis:0.56), (HYDROCHLOROTHIAZIDE\LISINOPRIL:Cystic fibrosis:0.62), (HYDROCHLOROTHIAZIDE\LOSARTAN POTASSIUM:Cystic fibrosis:0.39), (HYDROCHLOROTHIAZIDE\VALSARTAN:Cystic fibrosis:0.11), (HYDROXYCHLOROQUINE DIPHOSPHATE:Cystic fibrosis:0.08), (IBRUTINIB:Cystic fibrosis:0.58), (IMIPRAMINE HYDROCHLORIDE:Cystic fibrosis:0.42), (INDAPAMIDE\PERINDOPRIL:Cystic fibrosis:0.04), (INTERFERON GAMMA-1B:Cystic fibrosis:0.9), (ISOSORBIDE:Cystic fibrosis:0.29), (LENALIDOMIDE:Cystic fibrosis:0.05), (LERCANIDIPINE:Cystic fibrosis:0.09), (LERCANIDIPINE HYDROCHLORIDE:Cystic fibrosis:0.02), (LIOTHYRONINE SODIUM:Cystic fibrosis:0.29), (LOVASTATIN:Cystic fibrosis:1.42), (MAGNESIUM GLUCONATE:Cystic fibrosis:0.1), (MECLIZINE HYDROCHLORIDE:Cystic fibrosis:0.03), (MEPOLIZUMAB:Cystic fibrosis:1.39), (METHENAMINE HIPPURATE:Cystic fibrosis:0.52), (METHYLPREDNISOLONE ACETATE:Cystic fibrosis:1.04), (METHYLPREDNISOLONE SODIUM SUCCINATE:Cystic fibrosis:0.82), (METOLAZONE:Cystic fibrosis:0.04), (MIDODRINE:Cystic fibrosis:0.11), (MODAFINIL:Cystic fibrosis:0.65), (NABUMETONE:Cystic fibrosis:0.29), (NINTEDANIB:Cystic fibrosis:0.55), (NITROFURANTOIN\NITROFURANTOIN MONOHYDRATE:Cystic fibrosis:0.3), (OLODATEROL HYDROCHLORIDE\TIOTROPIUM BROMIDE MONOHYDRATE:Cystic fibrosis:0.41), (OMEGA-3-ACID ETHYL ESTERS:Cystic fibrosis:0.64), (PHENYTOIN:Cystic fibrosis:0.44), (PIMECROLIMUS:Cystic fibrosis:0.18), (PONATINIB:Cystic fibrosis:0.83), (PRAVASTATIN\PRAVASTATIN SODIUM:Cystic fibrosis:0.2), (PROGESTERONE:Cystic fibrosis:0.69), (RANOLAZINE:Cystic fibrosis:0.35), (ROPINIROLE HYDROCHLORIDE:Cystic fibrosis:0.55), (ROSUVASTATIN:Cystic fibrosis:0.17), (SECUKINUMAB:Cystic fibrosis:0.4), (TELMISARTAN:Cystic fibrosis:0.47), (TERBINAFINE HYDROCHLORIDE:Cystic fibrosis:0.01), (TERIFLUNOMIDE:Cystic fibrosis:0.04), (THEOPHYLLINE:Cystic fibrosis:0.68), (TIMOLOL:Cystic fibrosis:0.43), (TRIAMTERENE:Cystic fibrosis:0.21), (ZIPRASIDONE HYDROCHLORIDE\ZIPRASIDONE MESYLATE:Cystic fibrosis:0.03), (LUMACAFTOR:Cystic fibrosis lung:0.1), (TIOTROPIUM BROMIDE MONOHYDRATE:Cystic fibrosis lung:0.01), (.ALPHA.1-PROTEINASE INHIBITOR HUMAN:Cystitis:0.14), (ACARBOSE:Cystitis:0.49), (ACETAMINOPHEN\OXYCODONE\OX YCODONE HYDROCHLORIDE:Cystitis:0.53), (ACLIDINIUM BROMIDE:Cystitis:0.18), (ACTIVATED CHARCOAL:Cystitis:0.09), (AGALSIDASE BETA:Cystitis:0.45), (AMANTADINE:Cystitis:0.58), (AMANTADINE HYDROCHLORIDE:Cystitis:0.12), (AMIODARONE HYDROCHLORIDE:Cystitis:1.23), (AMISULPRIDE:Cystitis:0.12), (AMLODIPINE BESYLATE\ATORVASTATIN CALCIUM:Cystitis:0), (AMLODIPINE\VALSARTAN:Cystitis:0.01), (ATOVAQUONE:Cystitis: 0.34), (BICALUTAMIDE:Cystitis:0.61), (BISACODYL OR DOCUSATE SODIUM:Cystitis:1.25), (BRINZOLAMIDE:Cystitis:0.57), (BUDESONIDE\FORMOTEROL FUMARATE:Cystitis:0.35), (CALCIPOTRIENE:Cystitis:0.1), (CALCITONIN SALMON:Cystitis:0.24), (CALCIUM ACETATE:Cystitis:0.36), (CANAGLIFLOZIN\METFORMIN HYDROCHLORIDE:Cystitis:0.39), (CANAKINUMAB:Cystitis:0.49), (CANDESARTAN CILEXETIL\HYDROCHLOROTHIAZIDE:Cystitis:1.42), (CARFILZOMIB:Cystitis:1.05), (CA RVEDILOL PHOSPHATE:Cystitis:0.89), (CASPOFUNGIN:Cystitis:0.01), (CEFAZOLIN SODIUM:Cystitis:0.23), (CHLORAMBUCIL:Cystitis:0.09), (CHONDROITIN SULFATE (BOVINE)\GLUCOSAMINE HYDROCHLORIDE:Cystitis:0.16), (CLOBAZAM:Cystitis:0.03), (CORTICOTROPIN:Cystitis:00.23), (CRIZOTINIB:Cystitis:0.16), (DACLIZUMAB:Cystitis:1.4), (DALTEPARIN:Cystitis:0.5), (DANAZOL:Cystitis:0.06), (DAPAGLIFLOZIN PROPANEDIOL:Cystitis:0.32), (DAPSONE:Cystitis:0.16), (DARBEPOETIN ALFA:Cystitis:0.07), (DESONIDE:Cystitis:0.16), (DESVENLAFAXINE SUCCINATE:Cystitis:0.26), (DEVICE:Cystitis:0.35), (DEXAMETHASONE ACETATE:Cystitis:0.58), (DOXEPIN HYDROCHLORIDE:Cystitis:0.2), (DRONABINOL:Cystitis:0.68), (ECONAZOLE:Cystitis:0.42), (ELOTUZUMAB:Cystitis:0.07), (EMPAGLIFLOZIN\LINAGLIPTIN:Cystitis:0.21), (ESTROG ENS:Cystitis:0.01), (ETHINYL ESTRADIOL\NORGESTIMATE:Cystitis:0.23), (ETRETINATE:Cystitis:0.39), (EVOLOCUMA B:Cystitis:1.26), (EZETIMIBE\SIMVASTATIN:Cystitis:0.67), (FENOFIBRIC ACID:Cystitis:0.17), (FEXOFENADINE\PSEUDOEPHEDRINE:Cystitis:0.06), (FISH OIL\OMEGA-3 FATTY ACIDS:Cystitis:0.04), (FLAVIN ADENINE DINUCLEOTIDE:Cystitis:0.09), (FLUDROCORTISONE ACETATE:Cystitis:0.45), (FLUOCINONIDE:Cystitis:0.3), (FLUTICASONE FUROATE:Cystitis:0.67), (FORMOTEROL FUMARATE:Cystitis:0.65), (FORMOTEROL FUMARATE\MOMETASONE FUROATE:Cystitis:0.09), (FULVESTRANT:Cystitis:0.47), (FUSIDIC ACID:Cystitis:0.41), (GIMERACIL\OTERACIL\TEGAFUR:Cystitis:0.34), (GLYCOPYRRONI UM\INDACATEROL:Cystitis:0.46), (HERBALS\TURMERIC:Cystitis:0.48), (HYDROCORTIS ONE ACETATE:Cystitis:0.38), (HYDROXYCHLOROQUINE DIPHOSPHATE:Cystitis:0.39), (HYDROXYUREA:Cystitis:0.15), (ILOPROST:Cystitis:0.27), (I MATINIB MESYLATE:Cystitis:0.74), (IMIPRAMINE HYDROCHLORIDE:Cystitis:0.87), (INDOMETHACIN:Cystitis:0.72), (INFLIXIMAB-DYYB:Cystitis:0.21), (ISOSORBIDE:Cystitis:0.73), (IXAZOMIB:Cystitis:0), (LABETALOL HYDROCHLORIDE:Cystitis:0.27), (LACOSAMIDE:Cystitis:0.22), (LAPATINIB DITOSYLATE:Cystitis:0.19), (LEDIPASVIR\SOFOSBUVIR:Cystitis:0.85), (LEVOLEUCOVO RIN CALCIUM:Cystitis:0.66), (LITHIUM CARBONATE:Cystitis:1.49), (MEGESTROL ACETATE:Cystitis:0.51), (METFORMIN HYDROCHLORIDE\VILDAGLIPTIN:Cystitis:0.23), (METHIMAZOLE:Cystitis:0.81), (METH YLPREDNISOLONE ACETATE:Cystitis:1.24), (METOLAZONE:Cystitis:0.29), (MUPIROCIN\MUPIROCIN CALCIUM:Cystitis:0.26), (NADOLOL:Cystitis:0.32), (NADROPARIN CALCIUM:Cystitis:0.22), (NICORANDIL:Cystitis:0.1), (NINTEDANIB:Cystitis:0.29), (NIRAP ARIB:Cystitis:0.12), (NORETHINDRONE:Cystitis:0.63), (PAMIDRONATE DISODIUM:Cystitis:0.12), (PANITUMUMAB:Cystitis:0.43), (PAZOPANIB HYDROCHLORIDE:Cystitis:0.95), (PEMETREXED:Cystitis:0.25), (PILSICAINIDE HYDROCHLORIDE:Cystitis:0.1), (PONATINIB:Cystitis:0.18), (PRAZOSIN:Cystitis:0.07), (PR AZOSIN HYDROCHLORIDE:Cystitis:0.05), (PYRIDOSTIGMINE BROMIDE:Cystitis:0.15), (PYRIDOXINE:Cystitis:0.21), (RAMUCIRUMAB:Cystitis:0.05), (RIV ASTIGMINE TARTRATE:Cystitis:0.3), (SARPOGRELATE:Cystitis:0.28), (SITAGLIPTIN:Cystitis:0.43), (SO DIUM FERROUS CITRATE:Cystitis:0.19), (TETRACYCLINE:Cystitis:0.62), (TIOTROPIUM:Cystitis:0.49), (TOC OPHEROL:Cystitis:0.04), (TRICHLORMETHIAZIDE:Cystitis:0.29), (UMECLIDINIUM BROMIDE:Cystitis:0.1), (UMECLIDINIUM BROMIDE\VILANTEROL TRIFENATATE:Cystitis:0.53), (ZINC:Cystitis:0.56), (ACETAMINOPHEN\BUTALBITAL\CA FFEINE:Cystitis interstitial:0.91), (ACETAMINOPHEN\OXYCODONE\OXYCODONE HYDROCHLORIDE:Cystitis interstitial:0.01), (AMIODARONE:Cystitis interstitial:0.26), (AZITHROMYCIN DIHYDRATE:Cystitis interstitial:0.19), (BENAZEPRIL HYDROCHLORIDE:Cystitis interstitial:0.27), (CALCIUM CARBONATE\VITAMIN D:Cystitis interstitial:0.26), (CARVEDILOL PHOSPHATE:Cystitis interstitial:0.09), (CERTOLIZUMAB PEGOL:Cystitis interstitial:0.67), (CETIRIZINE HYDROCHLORIDE\PSEUDOEPHEDRINE HYDROCHLORIDE:Cystitis interstitial:0.05), (CLINDAMYCIN\CLINDAMYCIN PHOSPHATE:Cystitis interstitial:0.53), (COLCHICINE:Cystitis interstitial:0.13), (CYANOCOBALAMIN\DEXPANTHENOL\NIACINAMIDE\PYRIDOXINE HYDROCHLORIDE\RIBOFLAVIN 5′-PHOSPHATE SODIUM\THIAMINE HYDROCHLORIDE\VITAMIN B COMPLEX:Cystitis interstitial:0.15), (DACLIZUMAB:Cystitis interstitial:0.08), (DIMENHYDRINATE:Cystitis interstitial:0.03), (DIPHENHYDRAMINE:Cystitis interstitial:0.55), (DOCUSATE CALCIUM:Cystitis interstitial:0.01), (ENALAPRIL MALEATE:Cystitis interstitial:0.21), (ENOXAPARIN:Cystitis interstitial:0.12), (EVOLOCUMAB:Cystitis interstitial:0.86), (EZETIMIBE\SIMVASTATIN:Cystitis interstitial:0.14), (FERROUS SULFATE:Cystitis interstitial:0.51), (FINGOLIMOD HYDROCHLORIDE:Cystitis interstitial:0.16), (FISH OIL\OMEGA-3 FATTY ACIDS:Cystitis interstitial:0.03), (FLUTICASONE:Cystitis interstitial:0.2), (HERBALS\TURMERIC:Cystitis interstitial:0.33), (HYDROXYCHLOROQUINE DIPHOSPHATE:Cystitis interstitial:0.17), (IBRUTINIB:Cystitis interstitial:0.67), (INDOMETHACIN:Cystitis interstitial:0.19), (INSULIN ASPART:Cystitis interstitial:0.87), (INSULIN LISPRO:Cystitis interstitial:0.6), (ISOSORBIDE MONONITRATE:Cystitis interstitial:0.53), (MEDROXYPROGESTERONE ACETATE:Cystitis interstitial:0.82), (METHYLPREDNISOLONE ACETATE:Cystitis interstitial:0.26), (OLANZAPINE:Cystitis interstitial:0.44), (OMEGA-3-ACID ETHYL ESTERS:Cystitis interstitial:0.26), (OXYGEN:Cystitis interstitial:0.47), (PAROXETINE\PAROXETINE HYDROCHLORIDE:Cystitis interstitial:0.44), (PEGINTERFERON BETA-1A:Cystitis interstitial:0.13), (PENICILLIN:Cystitis interstitial:0.35), (PYRIDOXINE:Cystitis interstitial:0.17), (RANITIDINE:Cystitis interstitial:0.11), (RANOLAZINE:Cystitis interstitial:0.61), (RISEDRONATE SODIUM:Cystitis interstitial:0.46), (SOTALOL:Cystitis interstitial:0.17), (TERIFLUNOMIDE:Cystitis interstitial:0.83), (TOCILIZUMAB:Cystitis interstitial:0.11), (UMECLIDINIUM BROMIDE\VILANTEROL TRIFENATATE:Cystitis interstitial:0.31), (VARENICLINE TARTRATE:Cystitis interstitial:1.18), (ZINC:Cystitis interstitial:0.08), (LOPINAVIR:Cytomegalovirus chorioretinitis:0.3), (ANASTROZOLE:Cytomegalovirus infection:0.14), (ANIDULAFUNGIN:Cytomegalovirus infection:0.51), (ASCORBIC ACID:Cytomegalovirus infection:0.11), (BETAMETHASONE SODIUM PHOSPHATE:Cytomegalovirus infection:0.12), (BISACODYL OR DOCUSATE SODIUM:Cytomegalovirus infection:0.34), (BLESELUMAB:Cytomegalovirus infection:0.22), (BLINATUMOMAB:Cytomegalovirus infection:0.56), (BUPROPION:Cytomegalovirus infection:0.08), (BUPROPION HYDROCHLORIDE:Cytomegalovirus infection:0.61), (CALCIUM ACETATE:Cytomegalovirus infection:0.44), (CALCIUM\VITAMIN D:Cytomegalovirus infection:0.32), (CANDESARTAN CILEXETIL\HYDROCHLOROTHIAZIDE:Cytomegalovirus infection:0.47), (CEFPIROME SULFATE:Cytomegalovirus infection:0.26), (CLOFARABINE:Cytomegalovirus infection:0.15), (CLONIDINE:Cytomegalovirus infection:0.61), (CORTISONE\HYDROCORTISONE:Cytomegalovirus infection:0.01), (DANAZOL:Cytomegalovirus infection:0.33), (DASATINIB:Cytomegalovirus infection:0.58), (DESLORATADINE:Cytomegalovirus infection:0.38), (DICLOFENAC:Cytomegalovirus infection:0.01), (DIPYRIDAMOLE:Cytomegalovirus infection:0.04), (DIVALPROEX SODIUM:Cytomegalovirus infection:0.82), (ECONAZOLE:Cytomegalovirus infection:0.64), (EDOXABAN TOSYLATE:Cytomegalovirus infection:0.13), (ELOTUZUMAB:Cytomegalovirus infection:0.32), (EZETIMIBE:Cytomegalovirus infection:0.17), (FERROUS SULFATE\FERROUS SULFATE, DRIED:Cytomegalovirus infection:1.02), (FLECAINIDE:Cytomegalovirus infection:0.02), (FOSFOMYCIN:Cytomegalovirus infection:0.25), (GARENOXACIN:Cytomegalovirus infection:0.11), (GUSPERIMUS TRIHYDROCHLORIDE:Cytomegalovirus infection:0.97), (HYDRALAZINE HYDROCHLORIDE:Cytomegalovirus infection:0.01), (HYDROCHLOROTHIAZIDE\IRBESARTAN:Cytomegalovirus infection:0.05), (HYDROCODONE:Cytomegalovirus infection:0.01), (HYDROCORTISONE ACETATE:Cytomegalovirus infection:0.13), (HYDROMORPHONE:Cytomegalovirus infection:0.57), (HYDROXYUREA:Cytomegalovirus infection:0.68), (IBRITUMOMAB TIUXETAN:Cytomegalovirus infection:1.01), (INDAPAMIDE\PERINDOPRIL:Cytomegalovirus infection:0.38), (IPRATROPIUM:Cytomegalovirus infection:0.07), (LERCANIDIPINE HYDROCHLORIDE:Cytomegalovirus infection:0.38), (LITHIUM CARBONATE:Cytomegalovirus infection:0.09), (LOVASTATIN:Cytomegalovirus infection:0.49), (MAGNESIUM ASPARTATE HYDROCHLORIDE:Cytomegalovirus infection:0.1), (MELOXICAM:Cytomegalovirus infection:0.41), (METHENOLONE ENANTHATE:Cytomegalovirus infection:0.63), (METOPROLOL SUCCINATE:Cytomegalovirus infection:0.18), (MITOXANTRONE HYDROCHLORIDE:Cytomegalovirus infection:0.05), (MIZORIBINE:Cytomegalovirus infection:0), (MUROMONAB-CD3:Cytomegalovirus infection:0.23), (NOREPINEPHRINE:Cytomegalovirus infection:0.23), (OSELTAMIVIR:Cytomegalovirus infection:0.58), (OXCARBAZEPINE:Cytomegalovirus infection:1.27), (PAROXETINE\PAROXETINE HYDROCHLORIDE:Cytomegalovirus infection:0.09), (POLYETHYLENE GLYCOL 3350:Cytomegalovirus infection:0.29), (POLYMYXIN B SULFATE:Cytomegalovirus infection:0.25), (PREDNISOLONE SODIUM PHOSPHATE:Cytomegalovirus infection:0.43), (QUETIAPINE:Cytomegalovirus infection:0.24), (RALTEGRAVIR:Cytomegalovirus infection:0.45), (SECUKINUMAB:Cytomegalovirus infection:0.56), (SOTALOL:Cytomegalovirus infection:0.41), (THALIDOMIDE:Cytomegalovirus infection:0.9), (TIGECYCLINE:Cytomegalovirus infection:0.68), (TINZAPARIN SODIUM:Cytomegalovirus infection:0.65), (RIFAMPIN:Cytomegalovirus test positive:0.74), (GUSPERIMUS TRIHYDROCHLORIDE:Cytomegalovirus viraemia:0.25), (METRONIDAZOLE:Cytomegalovirus viraemia:0.19), (THALIDOMIDE:Cytomegalovirus viraemia:0.34), (VINCRISTINE SULFATE:Cytopenia:0.1), (.ALPHA.1-PROTEINASE INHIBITOR HUMAN:Decreased appetite:0.61), (ACARBOSE:Decreased appetite:0.99), (ACENOCOUMAROL:Decreased appetite:0.69), (ACETAMINOPHEN\CAFFEINE:Decreased appetite:0.12), (ACTIVATED CHARCOAL:Decreased appetite:0.59), (ADENINE:Decreased appetite:0.72), (AGALSIDASE BETA:Decreased appetite:0.15), (ALBUMIN BOVINE\TANNIC ACID:Decreased appetite:0.32), (ALCOHOL:Decreased appetite:0.65), (ALEMTUZUMAB:Decreased appetite:0.46), (ALFUZOSIN HYDROCHLORIDE:Decreased appetite:0.5), (ALUMINUM SILICATE:Decreased appetite:0.3), (AMILORIDE HYDROCHLORIDE\HYDROCHLOROTHIAZIDE:Decreased appetite:0.5), (AMINO ACIDS\CARBOHYDRATES\MINERALS\SOYBEAN OIL\VITAMINS:Decreased appetite:0.34), (AMISULPRIDE:Decreased appetite:0.71), (AMLODIPINE BESYLATE\CANDESARTAN CILEXETIL:Decreased appetite:0.18), (AMLODIPINE BESYLATE\IRBESARTAN:Decreased appetite:0.03), (AMLODIPINE\VALSARTAN:Decreased appetite:0.25), (AMMONIUM GLYCYRRHIZATE:Decreased appetite:0.24), (AMOXAPINE:Decreased appetite:0.51), (AMOXICILLIN\CLARITHROMYCIN\ESOMEPRAZOLE:Decreased appetite:0.02), (AMPICILLIN:Decreased appetite:0.87), (AMPICILLIN SODIUM:Decreased appetite:0.09), (ANTIHISTAMINES NOS:Decreased appetite:0.25), (ASPIRIN LYSINE:Decreased appetite:1.11), (ASPIRIN\LANSOPRAZOLE:Decreased appetite:0.09), (ASPIRIN\MAGNESIUM OXIDE:Decreased appetite:0.08), (AZELNIDIPINE:Decreased appetite:0.7), (BECLOMETHASONE:Decreased appetite:0.22), (BENAZEPRIL HYDROCHLORIDE:Decreased appetite:1.27), (BENAZEPRIL\BENAZEPRIL HYDROCHLORIDE:Decreased appetite:0.91), (BENIDIPINE HYDROCHLORIDE:Decreased appetite:0.45), (BENZTROPINE:Decreased appetite:0.03), (BENZTROPINE MESYLATE:Decreased appetite:0.07), (BEPRIDIL:Decreased appetite:0.54), (BETAMETHASONE SODIUM PHOSPHATE\NEOMYCIN SULFATE:Decreased appetite:0.04), (BETAMETHASONE\CALCIPOTRIENE:Decreased appetite:0.06), (BETAMIPRON\PANIPENEM:Decreased appetite:0.37), (BLINATUMOMAB:Decreased appetite:0.49), (BRIMONIDINE TARTRATE\TIMOLOL MALEATE:Decreased appetite:0.03), (BUDESONIDE\FORMOTEROL:Decreased appetite:0.05), (CABAZITAXEL:Decreased appetite:0.35), (CABOZANTINIB S-MALATE:Decreased appetite:0.06), (CALCIPOTRIENE:Decreased appetite:0.13), (CALCITONIN:Decreased appetite:0.25), (CALCITONIN SALMON:Decreased appetite:1.15), (CALCIUM CARBONATE\ERGOCALCIFEROL\RETINOL:Decreased appetite:0.27), (CALCIUM CITRATE:Decreased appetite:0.95), (CALCIUM\MAGNESIUM:Decreased appetite:0.39), (CAMPHOR\MENTHOL\METHYL SALICYLATE:Decreased appetite:0.39), (CANAGLIFLOZIN\METFORMIN HYDROCHLORIDE:Decreased appetite:0.83), (CANAKINUMAB:Decreased appetite:0.63), (CANDESARTAN:Decreased appetite:2.38), (CAPTOPRIL:Decreased appetite:0.83), (CARPERITIDE:Decreased appetite:0.32), (CARVEDILOL PHOSPHATE:Decreased appetite:1.11), (CASANTHRANOL\DOCUSATE SODIUM:Decreased appetite:0.38), (CEFDINIR:Decreased appetite:0.04), (CEFPIROME SULFATE:Decreased appetite:0.28), (CEPHARANTHINE:Decreased appetite:0.69), (CERTOLIZUMAB PEGOL:Decreased appetite:1.37), (CHIMAPHILA UMBELLATA\MANGANESE CHLORIDE\POPULUS TREMULOIDES\SODIUM TAUROCHOLATE:Decreased appetite:0.1), (CHLORTHALIDONE:Decreased appetite:0.81), (CHONDROITIN SULFATE (BOVINE)\GLUCOSAMINE HYDROCHLORIDE:Decreased appetite:0.09), (CIFENLINE:Decreased appetite:0.01), (CLOBAZAM:Decreased appetite:0.05), (CLOPERASTINE:Decreased appetite:0.24), (CLOZAPINE:Decreased appetite:1.53), (COLISTIMETHATE SODIUM:Decreased appetite:0.24), (COLISTIN:Decreased appetite:0.13), (DABRAFENIB MESYLATE:Decreased appetite:0.4), (DACLIZUMAB:Decreased appetite:1.45), (DANAZOL:Decreased appetite:0.64), (DANTROLENE SODIUM:Decreased appetite:0.07), (DAPAGLIFLOZIN PROPANEDIOL:Decreased appetite:0.12), (DAPTOMYCIN:Decreased appetite:0.2), (DASATINIB:Decreased appetite:1.97), (DEFLAZACORT:Decreased appetite:0.3), (DEVICE:Decreased appetite:0.26), (DILAZEP:Decreased appetite:0.7), (DIMEMORFAN PHOSPHATE:Decreased appetite:0.13), (DIPYRONE:Decreased appetite:0.09), (DISTIGMINE BROMIDE:Decreased appetite:0.41), (DOBUTAMINE HYDROCHLORIDE:Decreased appetite:0.52), (DOFETILIDE:Decreased appetite:0.48), (DOXYCYCLINE HYCLATE:Decreased appetite:0.39), (DUTASTERIDE\TAMSULOSIN HYDROCHLORIDE:Decreased appetite:0.11), (ECONAZOLE:Decreased appetite:0.61), (ELETRIPTAN HYDROBROMIDE:Decreased appetite:1.13), (EMPAGLIFLOZIN\LINAGLIPTIN:Decreased appetite:0.2), (ERIBULIN:Decreased appetite:0.76), (ESTROGENS, CONJUGATED\MEDROXYPROGESTERONE ACETATE:Decreased appetite:0.34), (ETRETINATE:Decreased appetite:1.5), (EVOLOCUMAB:Decreased appetite:0.83), (FERROUS FUMARATE:Decreased appetite:0.08), (FESOTERODINE FUMARATE:Decreased appetite:1.29), (FLAVIN ADENINE DINUCLEOTIDE:Decreased appetite:0.36), (FLUDROCORTISONE ACETATE:Decreased appetite:0.57), (FLUINDIONE:Decreased appetite:0.23), (FLUTICASONE FUROATE\SALMETEROL XINAFOATE:Decreased appetite:1.43), (FORMOTEROL FUMARATE:Decreased appetite:0.98), (FORMOTEROL FUMARATE\MOMETASONE FUROATE:Decreased appetite:0.56), (FOSFLUCONAZOLE:Decreased appetite:0.33), (FOSPHENYTOIN\FOSPHENYTOIN SODIUM:Decreased appetite:0.05), (FURSULTIAMINE:Decreased appetite:0.1), (FUSIDIC ACID:Decreased appetite:0.94), (GLYCOPYRROLATE:Decreased appetite:0.55), (GLYCOPYRRONIUM\INDACATEROL:Decreased appetite:1.06), (GUANFACINE HYDROCHLORIDE:Decreased appetite:0.23), (HYDROCHLOROTHIAZIDE\IRBESARTAN:Decreased appetite:1.39), (HYDROCHLOROTHIAZIDE\TELMISARTAN:Decreased appetite:0.57), (HYDROXYCHLOROQUINE DIPHOSPHATE:Decreased appetite:0), (HYDROXYZINE PAMOATE:Decreased appetite:1.69), (HYOSCYAMINE:Decreased appetite:0.49), (IGURATIMOD:Decreased appetite:0.17), (ILOPROST:Decreased appetite:0.87), (IMIDAPRIL:Decreased appetite:0.56), (IMIPENEM:Decreased appetite:0.53), (IMIPRAMINE HYDROCHLORIDE:Decreased appetite:1.11), (IMMUNE GLOBULIN NOS:Decreased appetite:0.31), (INDAPAMIDE\PERINDOPRIL:Decreased appetite:1.08), (INOTUZUMAB OZOGAMICIN:Decreased appetite:0.3), (INTERFERON:Decreased appetite:0.29), (INTERFERON ALFA-2A:Decreased appetite:0.49), (INTERFERON ALFA-2B:Decreased appetite:0.95), (INTERFERON GAMMA-1B:Decreased appetite:0.27), (KETOROLAC\KETOROLAC TROMETHAMINE:Decreased appetite:0.38), (LANREOTIDE ACETATE:Decreased appetite:0.18), (LEVALBUTEROL:Decreased appetite:0.21), (LIOTHYRONINE SODIUM:Decreased appetite:0.36), (LOMITAPIDE MESYLATE:Decreased appetite:0.19), (LUMACAFTOR:Decreased appetite:0.05), (LYSOZYME HYDROCHLORIDE:Decreased appetite:0.41), (MAXACALCITOL:Decreased appetite:0.49), (MEFENAMIC ACID:Decreased appetite:0.13), (MEPOLIZUMAB:Decreased appetite:0.38), (METAXALONE:Decreased appetite:1.17), (METHENAMINE HIPPURATE:Decreased appetite:0.82), (METHIMAZOLE:Decreased appetite:0.65), (METHYLPREDNISOLONE ACETATE:Decreased appetite:0.34), (MEXILETINE HYDROCHLORIDE:Decreased appetite:0.44), (MIDODRINE HYDROCHLORIDE:Decreased appetite:0.28), (MODAFINIL:Decreased appetite:0.95), (MOSAPRIDE CITRATE DIHYDRATE:Decreased appetite:0.08), (NADOLOL:Decreased appetite:0.56), (NALOXONE HYDROCHLORIDE\TILIDINE HYDROCHLORIDE:Decreased appetite:0.33), (NATEGLINIDE:Decreased appetite:0.45), (NORETHINDRONE:Decreased appetite:0.68), (OK-432:Decreased appetite:0.12), (OLOPATADINE:Decreased appetite:0.51), (PANTOPRAZOLE MAGNESIUM:Decreased appetite:1.51), (PATIROMER:Decreased appetite:0.19), (PAZUFLOXACIN MESILATE:Decreased appetite:0.02), (PENTAMIDINE ISETHIONATE:Decreased appetite:0.23), (PHENYTOIN:Decreased appetite:0.38), (PIMOBENDAN:Decreased appetite:0.48), (PIROXICAM:Decreased appetite:0.37), (POLYSTYRENE SULFONIC ACID:Decreased appetite:0.09), (PORFIMER SODIUM:Decreased appetite:0.13), (POTASSIUM CITRATE\TRISODIUM CITRATE DIHYDRATE:Decreased appetite:1.11), (POTASSIUM IODIDE:Decreased appetite:0.05), (PRAZOSIN HYDROCHLORIDE:Decreased appetite:0.29), (RADIATION THERAPY:Decreased appetite:1.44), (RALOXIFENE HYDROCHLORIDE:Decreased appetite:0.33), (RANIMUSTINE:Decreased appetite:0.08), (RETINOL:Decreased appetite:0.29), (RUCAPARIB CAMSYLATE:Decreased appetite:0.05), (SACUBITRIL\VALSARTAN:Decreased appetite:0.77), (SALMETEROL:Decreased appetite:0.4), (SALMETEROL XINAFOATE:Decreased appetite:0.17), (SECUKINUMAB:Decreased appetite:0.98), (SELEXIPAG:Decreased appetite:0.48), (SEVOFLURANE:Decreased appetite:0.44), (SIVELESTAT SODIUM:Decreased appetite:0.1), (SODIUM FLUORIDE:Decreased appetite:0.14), (SUMATRIPTAN\SUMATRIPTAN SUCCINATE:Decreased appetite:1.34), (SUPLATAST TOSILATE:Decreased appetite:0.69), (TAMOXIFEN CITRATE:Decreased appetite:0.42), (TEDUGLUTIDE\WATER:Decreased appetite:0.12), (TEGAFUR\URACIL:Decreased appetite:0.16), (TELOTRISTAT ETHYL:Decreased appetite:0.09), (TENELIGLIPTIN:Decreased appetite:0.15), (TERAZOSIN\TERAZOSIN HYDROCHLORIDE:Decreased appetite:0.09), (TERBUTALINE:Decreased appetite:0.33), (THEOPHYLLINE:Decreased appetite:0.12), (TOCOPHEROL:Decreased appetite:0.32), (TOCOPHERYL NICOTINATE, D-.ALPHA.:Decreased appetite:0.29), (TRAMETINIB DIMETHYL SULFOXIDE:Decreased appetite:0.4), (TRANDOLAPRIL:Decreased appetite:0.09), (VARENICLINE TARTRATE:Decreased appetite:1.84), (VINDESINE SULFATE:Decreased appetite:0.09), (VINORELBINE\VINORELBINE TARTRATE:Decreased appetite:0.61), (VITAMIN A:Decreased appetite:0.78), (ZAFIRLUKAST:Decreased appetite:0.05), (ZIPRASIDONE HYDROCHLORIDE\ZIPRASIDONE MESYLATE:Decreased appetite:0.05), (ZIV-AFLIBERCEPT:Decreased appetite:0.41), (CANDESARTAN CILEXETIL:Decubitus ulcer:0.03), (CLINDAMYCIN\CLINDAMYCIN PHOSPHATE:Decubitus ulcer:0.24), (DEXLANSOPRAZOLE:Decubitus ulcer:0.07), (DILTIAZEM HYDROCHLORIDE:Decubitus ulcer:0.27), (EPINEPHRINE:Decubitus ulcer:0.11), (FERROUS SULFATE:Decubitus ulcer:0.16), (FLUTICASONE PROPIONATE:Decubitus ulcer:0.22), (LOSARTAN:Decubitus ulcer:0.08), (MELOXICAM:Decubitus ulcer:0.06), (PANTOPRAZOLE:Decubitus ulcer:0.22), (SACUBITRIL\VALSARTAN:Decubitus ulcer:0.36), (TERIFLUNOMIDE:Decubitus ulcer:0.23), (.ALPHA.1-PROTEINASE INHIBITOR HUMAN:Deep vein thrombosis:0.94), (ABACAVIR SULFATE\DOLUTEGRAVIR SODIUM\LAMIVUDINE:Deep vein thrombosis:0.22), (ABIRATERONE:Deep vein thrombosis:0.25), (ABOBOTULINUMTOXINA:Deep vein thrombosis:0.04), (ACETAMINOPHEN\DICHLORALPHENAZONE\ISOMETHEPTENE:Deep vein thrombosis:0.14), (ACTIVATED CHARCOAL:Deep vein thrombosis:1.04), (AGALSIDASE BETA:Deep vein thrombosis:1.4), (ALBIGLUTIDE:Deep vein thrombosis:0.19), (ALDESLEUKIN:Deep vein thrombosis:1.01), (ALUMINUM HYDROXIDE\MAGNESIUM OXIDE\OXETHAZAINE:Deep vein thrombosis:0.04), (AMBROXOL\CLENBUTEROL:Deep vein thrombosis:0.06), (AMINO ACIDS\CARBOHYDRATES\MINERALS\SOYBEAN OIL\VITAMINS:Deep vein thrombosis:0.02), (AMINOPHYLLINE:Deep vein thrombosis:0.2), (AMINOSALICYLIC ACID:Deep vein thrombosis:0.77), (AMISULPRIDE:Deep vein thrombosis:1.79), (AMLODIPINE BESYLATE\IRBESARTAN:Deep vein thrombosis:0.3), (AMLODIPINE\VALSARTAN:Deep vein thrombosis:1.5), (AMMONIUM GLYCYRRHIZATE\GLYCINE\METHIONINE:Deep vein thrombosis:0.06), (AMOXAPINE:Deep vein thrombosis:0), (AMOXICILLIN\CLARITHROMYCIN\ESOMEPRAZOLE:Deep vein thrombosis:0.72), (AMPICILLIN SODIUM:Deep vein thrombosis:0.18), (ANHYDROUS CITRIC ACID\ASPIRIN\SODIUM BICARBONATE:Deep vein thrombosis:0.06), (ASENAPINE MALEATE:Deep vein thrombosis:0.06), (ASPARAGINASE:Deep vein thrombosis:0.21), (ASPIRIN\BUTALBITAL\CAFFEINE:Deep vein thrombosis:0.03), (ASPIRIN\CLOPIDOGREL:Deep vein thrombosis:0.63), (ASPIRIN\DIHYDROXYALUMINUM AMINOACETATE ANHYDROUS\MAGNESIUM CARBONATE:Deep vein thrombosis:0.69), (ASUNAPREVIR:Deep vein thrombosis:0.02), (ATORVASTATIN CALCIUM\EZETIMIBE:Deep vein thrombosis:0.17), (AZITHROMYCIN OR METHOTREXATE SODIUM:Deep vein thrombosis:0.55), (BECLOMETHASONE DIPROPIONATE\FORMOTEROL:Deep vein thrombosis:0.35), (BELATACEPT:Deep vein thrombosis:0.48), (BEPRIDIL:Deep vein thrombosis:0.82), (BETAMETHASONE DIPROPIONATE\CALCIPOTRIENE:Deep vein thrombosis:0.46), (BETAMETHASONE DIPROPIONATE\CALCIPOTRIENE\CALCIPOTRIENE HYDRATE:Deep vein thrombosis:0.8), (BETAMETHASONE\CALCIPOTRIENE:Deep vein thrombosis:0.86), (BIPERIDEN HYDROCHLORIDE:Deep vein thrombosis:0.52), (BOTULINUM TOXIN TYPE A:Deep vein thrombosis:0.22), (BUCILLAMINE:Deep vein thrombosis:0.2), (BUDESONIDE\FORMOTEROL FUMARATE:Deep vein thrombosis:1.35), (BUPRENORPHINE\NALOXONE:Deep vein thrombosis:0.04), (CABOZANTINIB:Deep vein thrombosis:0.34), (CALCITONIN:Deep vein thrombosis:1.52), (CALCIUM CARBONATE\ERGOCALCIFEROL\RETINOL:Deep vein thrombosis:1.02), (CALCIUM POLYCARBOPHIL:Deep vein thrombosis:0.82), (CALCIUM\MAGNESIUM\ZINC:Deep vein thrombosis:0.75), (CAPSAICIN:Deep vein thrombosis:0.37), (CARBASALATE:Deep vein thrombosis:0.03), (CARBOHYDRATES\FATTY ACIDS\MINERALS\PROTEIN\VITAMINS:Deep vein thrombosis:0.29), (CARMUSTINE:Deep vein thrombosis:0.54), (CARVEDILOL PHOSPHATE:Deep vein thrombosis:2.52), (CASPOFUNGIN ACETATE:Deep vein thrombosis:0.3), (CEFOTAXIME SODIUM:Deep vein thrombosis:1.78), (CEFOTAXIME\CEFOTAXIME SODIUM:Deep vein thrombosis:0.92), (CHLOROTHIAZIDE:Deep vein thrombosis:0.78), (CICLESONIDE:Deep vein thrombosis:1.1), (CIPROFIBRATE:Deep vein thrombosis:0.63), (CLADRIBINE:Deep vein thrombosis:0.26), (CLEMASTINE FUMARATE:Deep vein thrombosis:0.45), (CLODRONATE DISODIUM:Deep vein thrombosis:0.03), (CLOMIPRAMINE:Deep vein thrombosis:0.13), (CLOXACILLIN SODIUM:Deep vein thrombosis:0.38), (COBICISTAT\ELVITEGRAVIR\EMTRICITABINE\TENOFOVIR ALAFENAMIDE FUMARATE:Deep vein thrombosis:0.4), (COLCHICINE\DICYCLOMINE HYDROCHLORIDE:Deep vein thrombosis:0.09), (CYPROHEPTADINE:Deep vein thrombosis:2.04), (DABIGATRAN ETEXILATE:Deep vein thrombosis:0.37), (DACLIZUMAB:Deep vein thrombosis:1.88), (DANAZOL:Deep vein thrombosis:1.56), (DANTROLENE SODIUM:Deep vein thrombosis:0.6), (DAPAGLIFLOZIN PROPANEDIOL\METFORMIN HYDROCHLORIDE:Deep vein thrombosis:0.08), (DASABUVIR\OMBITASVIR\PARITAPREVIR\RITONAVIR:Deep vein thrombosis:0.61), (DEFERIPRONE:Deep vein thrombosis:0.11), (DEFLAZACORT:Deep vein thrombosis:1.74), (DESOGESTREL\ETHINYL ESTRADIOL:Deep vein thrombosis:0.1), (DEXAMETHASONE\TOBRAMYCIN:Deep vein thrombosis:0.18), (DEXTROAMPHETAMINE SULFATE:Deep vein thrombosis:0.86), (DEXTROMETHORPHAN HYDROBROMIDE\QUINIDINE SULFATE:Deep vein thrombosis:0.01), (DICLOXACILLIN:Deep vein thrombosis:0.38), (DIFLUNISAL:Deep vein thrombosis:0.05), (DIGITOXIN:Deep vein thrombosis:1.72), (DIPYRONE\PROMETHAZINE HYDROCHLORIDE:Deep vein thrombosis:0.21), (DOLASETRON MESYLATE:Deep vein thrombosis:1.09), (DOLUTEGRAVIR:Deep vein thrombosis:0.05), (DOXYLAMINE SUCCINATE:Deep vein thrombosis:0.77), (DULAGLUTIDE:Deep vein thrombosis:0.3), (ECONAZOLE:Deep vein thrombosis:2.21), (EMPAGLIFLOZIN\LINAGLIPTIN:Deep vein thrombosis:1.12), (EPERISONE HYDROCHLORIDE:Deep vein thrombosis:0.19), (EPIRUBICIN HYDROCHLORIDE:Deep vein thrombosis:0.42), (ERIBULIN:Deep vein thrombosis:1.04), (ERYTHROMYCIN\SULFISOXAZOLE:Deep vein thrombosis:0.01), (ESMOLOL HYDROCHLORIDE:Deep vein thrombosis:0.43), (ESTAZOLAM:Deep vein thrombosis:0.1), (ESTRADIOL\NORETHINDRONE ACETATE:Deep vein thrombosis:0.39), (ETHACRYNIC ACID:Deep vein thrombosis:0.14), (ETHIODIZED OIL:Deep vein thrombosis:0.52), (ETOPOSIDE PHOSPHATE:Deep vein thrombosis:0.62), (ETRETINATE:Deep vein thrombosis:0.83), (FERROUS BISGLYCINATE:Deep vein thrombosis:0.06), (FERROUS GLYCINE SULFATE:Deep vein thrombosis:0.13), (FERROUS SULFATE\GLYCINE:Deep vein thrombosis:0.64), (FLUOCINOLONE ACETONIDE:Deep vein thrombosis:0.59), (FLUOROURACIL\LEUCOVORIN CALCIUM\OXALIPLATIN:Deep vein thrombosis:0.95), (FLUTAMIDE:Deep vein thrombosis:0.51), (FORMOTEROL FUMARATE\MOMETASONE FUROATE:Deep vein thrombosis:0.81), (FOSFOMYCIN:Deep vein thrombosis:0.66), (GINGER:Deep vein thrombosis:0.12), (GLIPIZIDE\METFORMIN HYDROCHLORIDE:Deep vein thrombosis:0.14), (GLYCOPYRROLATE:Deep vein thrombosis:1.14), (GLYCOPYRROLATE\INDACATEROL MALEATE:Deep vein thrombosis:0.61), (HALOPERIDOL DECANOATE:Deep vein thrombosis:0.08), (HYDROCHLOROTHIAZIDE\QUINAPRIL HYDROCHLORIDE:Deep vein thrombosis:0.3), (HYDROCHLOROTHIAZIDE\SPIRONOLACTONE:Deep vein thrombosis:0.14), (IDARUCIZUMAB:Deep vein thrombosis:0.79), (IMIGLUCERASE:Deep vein thrombosis:0.83), (IMIPRAMINE HYDROCHLORIDE:Deep vein thrombosis:2.04), (INOTUZUMAB OZOGAMICIN:Deep vein thrombosis:0.76), (INTERFERON ALFA-2A:Deep vein thrombosis:1.77), (INTERFERON ALFA-2A OR INTERFERON ALFA-2B:Deep vein thrombosis:0.05), (INTERFERON ALFA-2B:Deep vein thrombosis:1.98), (INTERFERON GAMMA-1B:Deep vein thrombosis:0.58), (IODINE:Deep vein thrombosis:0.9), (ISOFLURANE:Deep vein thrombosis:0.19), (ISONIAZID\PYRAZINAMIDE\RIFAMPIN:Deep vein thrombosis:0.51), (ISOPROPYL ALCOHOL\LEUPROLIDE ACETATE\NORETHINDRONE ACETATE:Deep vein thrombosis:0.03), (IVACAFTOR:Deep vein thrombosis:0.44), (LANREOTIDE:Deep vein thrombosis:0.49), (LAPINE T-LYMPHOCYTE IMMUNE GLOBULIN:Deep vein thrombosis:0.18), (LEVOSIMENDAN:Deep vein thrombosis:0.02), (LEVOTHYROXINE\LIOTHYRONINE:Deep vein thrombosis:0.33), (LINSIDOMINE:Deep vein thrombosis:0.57), (LUTETIUM LU-177:Deep vein thrombosis:0.3), (MAGNESIUM ASPARTATE HYDROCHLORIDE:Deep vein thrombosis:0.96), (MAGNESIUM ASPARTATE\POTASSIUM ASPARTATE:Deep vein thrombosis:0.49), (MAGNESIUM PIDOLATE:Deep vein thrombosis:0.06), (MEPOLIZUMAB:Deep vein thrombosis:0.58), (METFORMIN HYDROCHLORIDE\SAXAGLIPTIN HYDROCHLORIDE:Deep vein thrombosis:0.42), (METHENOLONE ENANTHATE:Deep vein thrombosis:0.69), (METRELEPTIN:Deep vein thrombosis:0.3), (MIGLITOL:Deep vein thrombosis:0.28), (MIGLUSTAT:Deep vein thrombosis:0.15), (MOEXIPRIL HYDROCHLORIDE:Deep vein thrombosis:1), (MORPHINE SULFATE\NALTREXONE HYDROCHLORIDE:Deep vein thrombosis:1.08), (MOXONIDINE:Deep vein thrombosis:1.62), (NALBUPHINE HYDROCHLORIDE:Deep vein thrombosis:0.02), (NALOXONE HYDROCHLORIDE\TILIDINE HYDROCHLORIDE:Deep vein thrombosis:2.42), (NEOSTIGMINE:Deep vein thrombosis:0.34), (OSPEMIFENE:Deep vein thrombosis:1.39), (OXAPROZIN:Deep vein thrombosis:0.15), (OXYBUTYNIN\OXYBUTYNIN CHLORIDE:Deep vein thrombosis:0.18), (PALIPERIDONE PALMITATE:Deep vein thrombosis:0.81), (PANTOPRAZOLE MAGNESIUM:Deep vein thrombosis:2.45), (PATIROMER:Deep vein thrombosis:0.71), (PEGINTERFERON ALFA-2A OR PEGINTERFERON ALFA-2B OR PEGINTERFERON BETA-1A OR PEGINTERFERON LAMBDA-1A:Deep vein thrombosis:0.1), (PEGINTERFERON BETA-1A:Deep vein thrombosis:1.36), (PEGVISOMANT:Deep vein thrombosis:0.42), (PENTOSAN POLYSULFATE SODIUM:Deep vein thrombosis:1.2), (PENTOSTATIN:Deep vein thrombosis:0.91), (PHENAZOPYRIDINE HYDROCHLORIDE:Deep vein thrombosis:0.4), (PHENOBARBITAL SODIUM:Deep vein thrombosis:0.01), (PHLOROGLUCINOL\1,3,5-TRIMETHOXYBENZENE:Deep vein thrombosis:0.98), (PIMOBENDAN:Deep vein thrombosis:0.68), (PNEUMOCOCCAL VACCINE, POLYVALENT 23:Deep vein thrombosis:0.58), (POLAPREZINC:Deep vein thrombosis:0.08), (POLYETHYLENE GLYCOL 3350\POTASSIUM CHLORIDE\SODIUM BICARBONATE\SODIUM CHLORIDE\SODIUM SULFATE ANHYDROUS:Deep vein thrombosis:0.61), (PORFIMER SODIUM:Deep vein thrombosis:0.15), (PROTRIPTYLINE HYDROCHLORIDE:Deep vein thrombosis:0.2), (QUINACRINE:Deep vein thrombosis:0.08), (RALTEGRAVIR POTASSIUM:Deep vein thrombosis:0.76), (RIFABUTIN:Deep vein thrombosis:0.27), (RILPIVIRINE HYDROCHLORIDE:Deep vein thrombosis:0.18), (RUCAPARIB CAMSYLATE:Deep vein thrombosis:0.33), (SELEGILINE HYDROCHLORIDE:Deep vein thrombosis:0.45), (SELEXIPAG:Deep vein thrombosis:0.64), (SIBUTRAMINE HYDROCHLORIDE:Deep vein thrombosis:0.01), (STREPTOMYCIN\STREPTOMYCIN SULFATE:Deep vein thrombosis:0.36), (SUCCINYLCHOLINE CHLORIDE:Deep vein thrombosis:0.1), (SUFENTANIL CITRATE:Deep vein thrombosis:0.38), (TAFAMIDIS MEGLUMINE:Deep vein thrombosis:0.24), (TAFLUPROST:Deep vein thrombosis:0.35), (TELOTRISTAT ETHYL:Deep vein thrombosis:0.9), (TERIFLUNOMIDE:Deep vein thrombosis:0.82), (TESTOSTERONE ENANTHATE:Deep vein thrombosis:0.06), (TETRACYCLINE:Deep vein thrombosis:2), (THEOPHYLLINE:Deep vein thrombosis:0.6), (THIOPENTAL:Deep vein thrombosis:0.13), (THIOPENTAL SODIUM:Deep vein thrombosis:0.14), (TILIDINE:Deep vein thrombosis:0.04), (TIMOLOL MALEATE\TRAVOPROST:Deep vein thrombosis:0.22), (TREPROSTINIL DIOLAMINE:Deep vein thrombosis:0.07), (TRIMETAZIDINE:Deep vein thrombosis:0.08), (TRIPTORELIN PAMOATE:Deep vein thrombosis:0.61), (UMECLIDINIUM BROMIDE\VILANTEROL TRIFENATATE:Deep vein thrombosis:1.26), (VECURONIUM BROMIDE:Deep vein thrombosis:0.43), (VINORELBINE TARTRATE:Deep vein thrombosis:1.41), (XIPAMIDE:Deep vein thrombosis:0.65), (ZICONOTIDE ACETATE:Deep vein thrombosis:0.23), (ZILEUTON:Deep vein thrombosis:0.26), (ZIV-AFLIBERCEPT:Deep vein thrombosis:1.01), (ACETAMINOPHEN\BUTALBITAL\CAFFEINE:Dehydration:0.77), (ACET AMINOPHEN\OXYCODONE\OXYCODONE HYDROCHLORIDE:Dehydration:0.35), (ACETAMINOPHEN\TRAMADOL:Dehydration:0.06), (ALBUTEROL\IPRATROPIUM:Dehydration:0), (ALCOHOL:Dehydration:0.03), (ALDESLE UKIN:Dehydration:0.01), (AMISULPRIDE:Dehydration:0.62), (AMLODIPINE\VALSARTAN: Dehydration:0.09), (ANASTROZOLE:Dehydration:1.68),(ARMODAFINIL:Dehydration:0.21), (BECLOMETHASONE DIPROPIONATE:Dehydration:0.39), (BENAZEPRIL HYDROCHLORIDE:Dehydration:0.75), (BENAZEPRIL\BENAZEPRIL HYDROCHLORIDE:Dehydration:0.36), (BICALUTAMIDE:Dehydration:0.18), (BLINATUMO MAB:Dehydration:0.11), (BUDESONIDE\FORMOTEROL FUMARATE:Dehydration:0.06), (BUPRENORPHINE HYDROCHLORIDE:Dehydration:0.59), (BUPRENORPHINE HYDROCHLORIDE\NALOXONE HYDROCHLORIDE:Dehydration:0.06), (CALCITONIN SALMON:Dehydration:0.54), (CANAGLIFLOZIN\METFORMIN HYDROCHLORIDE:Dehydration:0.17), (CANDESARTAN CILEXETIL\HYDROCHLOROTHIAZIDE:Dehydration:0.78), (CARVEDILOL PHOSPHATE:Dehydration:0.54), (CEFEPIME\CEFEPIME HYDROCHLORIDE:Dehydration:0.23), (CEFOTAXIME SODIUM:Dehydration:0.78), (CHLORAMBUCIL:Dehydration:0.26), (COLISTIMETHATE SODIUM:Dehydration:0.04), (CORTISONE\HYDROCORTISONE:Dehydration:0.82), (DACLI ZUMAB:Dehydration:0.43), (DANAZOL:Dehydration:0), (DASATINIB:Dehydration:1.23), (DE SLORATADINE:Dehydration:0.4), (DEVICE:Dehydration:0.26), (DEXLANSOPRAZOLE:Dehydration: 0.89), (DIGITOXIN:Dehydration:0.18), (DIPYRONE:Dehydration:0.12), (DIVALPROEX SODIUM:Dehydration:1.05), (DOBUTAMINE HYDROCHLORIDE:Dehydration:0.1), (DOCUSATE CALCIUM:Dehydration:0.37), (DOLASETRON MESYLATE:Dehydration:0.09), (DULOXETINE:Dehydration:0.64), (EDOXABAN TOSYLATE:Dehydration:0.65), (ELETRIPTAN HYDROBROMIDE:Dehydration:0.45), (ENALAPRIL MALEATE:Dehydration:1.1), (EPIRUBICIN:Dehydration:0.94), (EPLERENONE:Dehydration:00.25), (ERLOTINIB:Dehydration:0.36), (EVOLOCUMAB:Dehydration:0.14), (EXEMESTANE: Dehydration:0.52), (FLECAINIDE:Dehydration:0.75), (FLUOCINONIDE:Dehydration:0.36), (FLU OXETINE\FLUOXETINE HYDROCHLORIDE:Dehydration:0.53), (FLUTICASONE:Dehydration:0.62), (FORMOTEROL: Dehydration:0.39), (FULVESTRANT:Dehydration:0.37), (FUSIDIC ACID:Dehydration:0.17), (GEFITINIB:Dehydration:0.2), (GIMERACIL\OTERACIL\TEGAFUR:Dehydration:0.67), (GLIPIZIDE:Dehydration:0.67), (GLYCOPYRRONIUM\lNDACATEROL: Dehydration:0.45), (HERBALS\TURMERIC:Dehydration:0.15), (HYDROCHLOROTHIAZIDE\IRBESARTAN:Dehydration:0.58), (HYDROCHLOROTHIAZIDE\LOSARTAN POTASSIUM:Dehydration:0.4), (HYDROXYCHLOROQUINE SULFATE:Dehydration:0.45), (ILOPROST:Dehydration:0.09), (IMATINIB MESYLATE:Dehydration:1.21), (IMIPRAMINE HYDROCHLORIDE:Dehydration:0.31), (INDAPAMIDE\PERINDOPRIL:Dehydration:0.38), (I RINOTECAN HYDROCHLORIDE:Dehydration:0.93), (LACTOBACILLUS RHAMNOSUS:Dehydration:0.35), (LAPATINIB DITOSYLATE:Dehydration:0.28), (LEDIPASVIR\SOFOSBUVIR:Dehydration:0.5), (LERCANI DIPINE:Dehydration:1.12), (LERCANIDIPINE HYDROCHLORIDE:Dehydration:0.29), (LEVOLEUCOVORIN CALCIUM:Dehydration:0.64), (LISDEXAMFETAMINE DIMESYLATE:Dehydration:0.03), (MECLIZINE HYDROCHLORIDE:Dehydration:0.08), (METAXALONE:Dehydration:0.29), (METFORMIN HYDROCHLORIDE\VILDAGLIPTIN:Dehydration:0.12), (METHENAMINE HIPPURATE:Dehydration:0.53), (METHENOLONE ENANTHATE:Dehydration:0.07), (METHYLPREDNISOLONE ACETATE:Dehydration:0.28), (MUPIROCIN\MUPIROCIN CALCIUM:Dehydration:0.07), (NABUMETONE:Dehydration:0.07), (NEBIVOLOL:Dehydration: 0.35), (NITROFURANTOIN:Dehydration:1.25), (OLAPARIB:Dehydration:0.57), (OLODATE ROL HYDROCHLORIDE\TIOTROPIUM BROMIDE MONOHYDRATE:Dehydration:0.36), (OXCARBAZEPINE:Dehydration:1.32), (OXYBUTYNI N CHLORIDE:Dehydration:0.29), (PANITUMUMAB:Dehydration:0.3), (PANTOPRAZOLE MAGNESIUM:Dehydration:0.79), (PAROXETINE:Dehydration:0.52), (PAZOPANIB HYDROCHLORIDE:Dehydration:0.67), (PEGINTERFERON BETA-1A:Dehydration:0.04), (PENTOSTATIN:Dehydration:0.11), (PRAVASTATIN SODIUM:Dehydration:1.29), (PRIMIDONE:Dehydration:1.02), (RADIATION THERAPY:Dehydration:0.49), (RANOLAZINE:Dehydration:0.67), (RIZATRIPTAN BENZOATE:Dehydration:0.29), (ROFLUMILAST:Dehydration:0.57), (ROPINIROLE HYDROCHLORIDE:Dehydration:0.17), (SECUKINUMAB:Dehydration:0.4), (SEVELAMER HYDROCHLORIDE:Dehydration:0.04), (SODIUM CITRATE:Dehydration:0.12), (SODIUM OXYBATE:Dehydration:0.13), (SOTALOL:Dehydration:0.9), (SUMATRIPTAN\SUMATRIPT AN SUCCINATE:Dehydration:0.43), (TERBINAFINE HYDROCHLORIDE:Dehydration:0.11), (TERBUTALINE SULFATE:Dehydration:0.12), (TERIFLUNOMIDE:Dehydration:0.54), (THALIDOMIDE:Dehydration: 1.82), (TICAGRELOR:Dehydration:0.07), (TIGECYCLINE:Dehydration:0.47), (TIMOLO L:Dehydration:0.33), (TINZAPARIN:Dehydration:0.02), (TIOTROPIUM:Dehydration:0.51), (TR ABECTEDIN:Dehydration:0.02), (UMECLIDINIUM BROMIDE\VILANTEROL TRIFENATATE:Dehydration:0.32), (VARENICLINE TARTRATE:Dehydration:0.81), (VINORELBINE TARTRATE:Dehydration:0.38), (ZINC:Dehydration:0.01), (ACARBOSE:Delirium:0.15), (ACET AZOLAMIDE:Delirium:0.35), (ALCOHOL:Delirium:0.72), (AMOXICILLIN:Delirium:0.55), (A MPICILLIN:Delirium:0.11), (ANASTROZOLE:Delirium:0.28), (APREPITANT:Delirium:0.1), (ASPIRIN LYSINE:Delirium:0.13), (BENZTROPINE:Delirium:0.04), (BERAPROST SODIUM:Delirium:0.07), (BRINZOLAMIDE:Delirium:0.16), (CALCIUM\VITAMIN D:Delirium:0.07), (CANDESARTAN CILEXETIL\HYDROCHLOROTHIAZIDE:Delirium:0.47), (CAPTOPRIL:Delirium:0.1), (CARF ILZOMIB:Delirium:0.28), (CARVEDILOL PHOSPHATE:Delirium:0.11), (CEFAZOLIN:Delirium:0.44), (CEFOTAXIME SODIUM:Delirium:0.5), (CERTOLIZUMAB PEGOL:Delirium:0.46), (CHOLESTYRAMINE:Delirium:0.41), (CLOBAZAM:Delirium:0.02), (CYCLOBENZAPRINE:Delirium:0.19), (DASATINIB:Delirium:0.33), (DOXAZOSIN MESYLATE:Delirium:0.1), (EPLERENONE:Delirium:0.22), (EZETIMIBE:Delirium:0.41), (FER ROUS SULFATE\FERROUS SULFATE, DRIED:Delirium:0.61), (FLUOXETINE\FLUOXETINE HYDROCHLORIDE:Delirium:0.24), (FLUTICASONE:Delirium:0.16), (FLUTICASONE FUROATE\SALMETEROL XINAFOATE:Delirium:0.14), (GLIPIZIDE:Delirium:0.11), (GLYCOPYRRONIUM\INDACAT EROL:Delirium:0.06), (HEPARIN CALCIUM:Delirium:0.02), (HYDROCHLOROTHIAZIDE\IRBESARTAN:Delirium:0.02), (HY DROCHLOROTHIAZIDE\LISINOPRIL:Delirium:0.06), (HYDROXYZINE PAMOATE:Delirium:0.13), (IBRUTINIB:Delirium:0.4), (ILOPROST:Delirium:0.16), (INDAPA MIDE:Delirium:0.08), (LABETALOL\LABETALOL HYDROCHLORIDE:Delirium:0.36), (LERCANIDIPINE:Delirium:0.62), (LEVALBUTEROL HYDROCHLORIDE:Delirium:0.14), (LOVASTATIN:Delirium:0.26), (MOXIFLOXACIN HYDROCHLORIDE:Delirium:1.1), (MYCOPHENOLATE MOFETIL\MYCOPHENOLATE MOFETIL HYDROCHLORIDE:Delirium:0.03), (NADROPARIN CALCIUM:Delirium:0.27), (NILOTINIB:Delirium:1.02), (PAROXETINE\PAROXETINE HYDROCHLORIDE:Delirium:0.36), (PAZOPANIB HYDROCHLORIDE:Delirium:0.13), (PHENOBARBITAL:Delirium:0.61), (PHENYTOIN SODIUM:Delirium:0.16), (POLYETHYLENE GLYCOLS:Delirium:1.19), (POSACONAZOLE:Delirium:0), (PRAMIPEXOLE DIHYDROCHLORIDE:Delirium:0.56), (RABEPRAZOLE:Delirium:0.05), (RANOLAZINE:Delirium: 0.61), (RIVASTIGMINE TARTRATE:Delirium:0.26), (SOTALOL:Delirium:0.35), (THALIDOMIDE:Delirium:0.94), (TIN ZAPARIN SODIUM:Delirium:0.48), (TRIAMCINOLONE ACETONIDE:Delirium:0.46), (VALGANCICLOVIR HYDROCHLORIDE:Delirium:0.27), (ACARBOSE:Delusion:0.09), (ACENOCOUMAROL:Delusion: 0.02), (ACETAMINOPHEN\OXYCODONE\OXYCODONE HYDROCHLORIDE:Delusion:0.33), (ALFACALCIDOL:Delusion:0.14), (AMITRIPTYLINE HYDROCHLORIDE:Delusion:0.46), (BENSERAZIDE\LEVODOPA:Delusion:0.5), (BUDESO NIDE\FORMOTEROL FUMARATE:Delusion:0.04), (BUPRENORPHINE HYDROCHLORIDE:Delusion:1.05), (CANDESARTAN:Delusion:0.91), (CANDESARTAN CILEXETIL\HYDROCHLOROTHIAZIDE:Delusion:0.49), (CARVEDILOL PHOSPHATE:Delusion:0.91), (CERTOLIZUMAB PEGOL:Delusion:0.33), (CHLORTHALIDONE:Delusion:0.52), (CHOLESTYRAMINE:Delusion: 0.63), (CLINDAMYCIN\CLINDAMYCIN PHOSPHATE:Delusion:0.54), (CLONIDINE HYDROCHLORIDE:Delusion:0.21), (CLOTRIMAZOLE:Delusion:0.22), (CORTISONE\HYDR OCORTISONE:Delusion:0.17), (DACLIZUMAB:Delusion:0.21), (DAPAGLIFLOZIN PROPANEDIOL:Delusion:0.3), (DEVICE:Delusion:0.33), (DEXTROAMPHETAMINE SULFATE:Delusion:0.01), (DOFETILIDE:Delusion:0.13), (EDOXABAN TOSYLATE:Delusion:0.05), (ELETRIPTAN HYDROBROMIDE:Delusion:0.87), (EMPAGLIFLOZIN\LINAGLIPTIN:Delusion:0.08), (ENO XAPARIN:Delusion:0.8), (EPINEPHRINE:Delusion:0.53), (ERYTHROMYCIN:Delusion:0.09), (EVEROLIMUS:Delusion:0.27), (EVOLOCUMAB:Delusion:0.84), (FERROUS SULFATE\FERROUS SULFATE, DRIED:Delusion:0.98), (FLUPHENAZINE:Delusion:0.05), (FLUTICASONE FUROATE:Delusion:0), (FLUTICASONE FUROATE\SALMETEROL XINAFOATE:Delusion:0.24), (GLICLAZIDE:Delusion:0.09), (GUANFACINE HYDROCHLORIDE:Delusion:0.5), (HEPARIN SODIUM:Delusion:0.32), (HYALURONATE SODIUM:Delusion:0.13), (HYDROCHLOROTHIAZIDE\IRBESARTAN:Delusion:0.33), (HYD ROCHLOROTHIAZIDE\TELMISARTAN:Delusion:0.16), (HYDROCHLOROTHIAZIDE\VA LSARTAN:Delusion:0.85), (HYDROMORPHONE:Delusion:0.13), (HYOSCYAMINE:Delusion: 0.36), (IBRUTINIB:Delusion:0.01), (INDAPAMIDE:Delusion:0.43), (INDOMETHACIN:Delusion: 0.07), (LERCANIDIPINE:Delusion:0.16), (LIDOCAINE HYDROCHLORIDE:Delusion:0.2), (LISDEXAMFETAMINE DIMESYLATE:Delusion:1.45), (METAXALONE:Delusion:1.04), (METHADONE HYDROCHLORIDE:Delusion:0.69), (METHYLPREDNISOLONE ACETATE:Delusion:0.28), (METHYLPREDNISOLONE SODIUM SUCCINATE:Delusion:0.7), (METRONIDAZOLE:Delusion:0.46), (MOMETASONE FUROATE:Delusion:0.31), (NALOXONE HYDROCHLORIDE\OXYCODONE HYDROCHLORIDE:Delusion:0.37), (OXYMORPHONE:Delusion:0.04), (PANTOPRAZOLE MAGNESIUM:Delusion:0.31), (PAROXETINE\PAROXETINE HYDROCHLORIDE:Delusion:1.11), (PRAZOSIN:Delusion:0.49), (PRAZOSIN HYDROCHLORIDE:Delusion:0.06), (PREDNISOLONE ACETATE:Delusion:0.61), (RABEPRAZOLE:Delusion:0.46), (RABEPRAZOLE SODIUM:Delusion:0.59), (RIZATRIPTAN BENZOATE:Delusion:0.39), (SACUBITRIL\VALSARTAN:Delusion:0.29), (SODIUM OXYBATE:Delusion:1.56), (SUMATRIPTAN\SUMATRIPTAN SUCCINATE:Delusion:0.44), (TELMISARTAN:Delusion:0.88), (TERIFLUNOMIDE:Delusion: 0.8), (TIOTROPIUM:Delusion:0.14), (TORSEMIDE:Delusion:0.84), (VALACYCLOVIR HYDROCHLORIDE:Delusion:0.3), (.ALPHA.1-PROTEINASE INHIBITOR HUMAN:Dementia:0.34), (ACETAMINOPHEN\OXYCODONE\OXYCODONE HYDROCHLORIDE:Dementia:0.65), (ACTIVATED CHARCOAL:Dementia:0.25), (ADENINE:Dementia:0.25), (ADO-TRASTUZUMAB EMTANSINE:Dementia:0.21), (AGALSIDASE BETA:Dementia:0.68), (ALCOHOL:Dementia:0.54), (AMISULPRIDE:Dementia:1.11), (AMLO DIPINE\VALSARTAN:Dementia:0.24), (AMOXAPINE:Dementia:0.33), (AMPHETAMINE ASPARTATE\AMPHETAMINE SULFATE\DEXTROAMPHETAMINE SACCHARATE\DEXTROAMPHETAMINE SULFATE:Dementia:1.32), (APREPITANT:Dementia:0.15), (AXITINIB:Dementia:0.41), (BEPR IDIL:Dementia:0.62), (BLONANSERIN:Dementia:0.06), (BRINZOLAMIDE:Dementia:0.9), (BU TYLSCOPOLAMINE BROMIDE:Dementia:0.22), (CALCITONIN SALMON:Dementia:0.04), (CALCIUM ACETATE:Dementia:1.18), (CANAGLIFLOZIN:Dementia:0.54), (CANAGLIFLOZIN\METFO RMIN HYDROCHLORIDE:Dementia:0.28), (CARVEDILOL PHOSPHATE:Dementia:1.51), (CERTOLIZUMAB PEGOL:Dementia:1.19), (CHONDROITIN SULFATE (BOVINE)\GLUCOSAMINE HYDROCHLORIDE:Dementia:0.12), (CILASTATIN SODIUM\IMIPENEM:Dementia:0.12), (CIMETIDINE:Dementia:0.81), (CLOMIPRAMINE HYDROCHLORIDE:Dementia:0.44), (CORTICOTROPIN:Dementia:0.48), (CYPROHEPTADI NE:Dementia:0.01), (DACLIZUMAB:Dementia:0.49), (DANAZOL:Dementia:0.24), (DAPAGLI FLOZIN PROPANEDIOL:Dementia:0.49), (DEFLAZACORT:Dementia:0.89), (DEXAMETHASONE SODIUM PHOSPHATE:Dementia:0.04), (DIGITOXIN:Dementia:0.32), (DIMENHYDRINATE:Dementia: 0.75), (DOXEPIN:Dementia:0.68), (DOXEPIN HYDROCHLORIDE:Dementia:0.38), (DRONABINOL:Dementia:0.25), (ECONAZOLE:Dementia: 0.34), (ELETRIPTAN HYDROBROMIDE:Dementia:0.97), (EMPAGLIFLOZIN\LINAGLIPTIN:Dementia:0), (EPERI SONE HYDROCHLORIDE:Dementia:0.07), (ESTROGENS, CONJUGATED\MEDROXYPROGESTERONE ACETATE:Dementia:0.1), (ETORICOXIB:Dementia:0.11), (ETRETINATE:Dementia:0.34), (FE RROUS GLYCINE SULFATE:Dementia:0.06), (FLUOCINONIDE:Dementia:0.13), (FORMOTEROL:Dementia:0.32), (FORMOTEROL FUMARATE DIHYDRATE\MOMETASONE FUROATE:Dementia:0.91), (FORMOTEROL FUMARATE\MOMETASONE FUROATE:Dementia:0.36), (FURSULTIAMINE:Dementia:0.13), (FUSIDIC ACID:Dementia:0.24), (GIMERACIL\OTERACIL\TEGAFUR:Dementia:0.2), (GLYCOPYRRO LATE\INDACATEROL MALEATE:Dementia:0.02), (GLYCOPYRRONIUM\INDACATEROL:Dementia:1.13), (GUAN FACINE:Dementia:0.58), (GUANFACINE HYDROCHLORIDE:Dementia:0.29), (IBRUTINIB:Dementia:1.17), (ILOPROST:Dementia:1.14), (INFLUENZA VIRUS VACCINE:Dementia:0.43), (INSULIN GLULISINE:Dementia:0.19), (INTERFERON:Dementia:0.24), (IRINOTECAN HYDROCHLORIDE:Dementia:0.93), (IRON SUCROSE:Dementia:0.62), (ISTRADEFYLLINE:Dementia:0.4), (LACTOBACILLUS RHAMNOSUS:Dementia:0.15), (LERCANIDIPINE HYDROCHLORIDE:Dementia:0.81), (LEVALBUTEROL HYDROCHLORIDE:Dementia:1.38), (LIDOCAINE\PRILOCAINE:Dementia:0.07), (LOMITAP IDE MESYLATE:Dementia:0.32), (LOXOPROFEN:Dementia:0.07), (MEPOLIZUMAB:Dementia:0.24), (METAMIZOLE:Dementia:0.46), (METHENAMINE HIPPURATE:Dementia:0.88), (METHOCARBAMOL:Dementia:0.78), (METHYLPREDNISOL ONE ACETATE:Dementia:0.47), (MOXIFLOXACIN:Dementia:0.2), (NADOLOL:Dementia:0.58), (N ADROPARIN CALCIUM:Dementia:0.35), (NALOXONE HYDROCHLORIDE\OXYCODONE HYDROCHLORIDE:Dementia:1.37), (NALTREXONE:Dementia:0.42), (NIRAPARIB:Dementia: 0.39), (ONABOTULINUMTOXINA:Dementia:0.02), (ORPHENADRINE:Dementia:0.08), (PE GINTERFERON BETA-1A:Dementia:0.69), (PEMETREXED DISODIUM:Dementia:0.76), (PENTOSAN POLYSULFATE SODIUM:Dementia:0.03), (PHYTONADIONE:Dementia:0.34), (PILSICAINIDE HYDROCHLORIDE:Dementia:0.96), (PIMOBENDAN:Dementia:0.04), (PIRIBEDIL:Dementia: 0.01), (PIROXICAM:Dementia:0.41), (PONATINIB:Dementia:0.49), (PRAZOSIN HYDROCHLORIDE:Dementia:0.09), (PREDNISOLONE ACETATE:Dementia:0.6), (PROPRANOLOL\PROPRANOLOL HYDROCHLORIDE:Dementia:0.83), (REGORAFENIB:Dementia:0.25), (RIZATRIPTAN BENZOATE:Dementia:0.6), (SAFINAMIDE:Dementia:0.12), (SALMETEROL:Dementia:0.07), (SCOPOLAMINE:Dementia:0.88), (SECUKINUMAB:Dementia:0.65), (SOTALOL HYDROCHLORIDE:Dementia:0.06), (SUMATRIPTAN:Dementia:0.47), (TAPENTADOL HYDROCHLORIDE:Dementia:0.19), (TETRACYCLINE:Dementia:0.06), (TINZAPARIN SODIUM:Dementia:0.03), (TOCILIZUMAB:Dementia:0.19), (TRANDOLAPRIL:Dementia:0.1), (TRIHEXYPHENIDYL:Dementia:0.23), (TRIMIPRAMINE MALEATE:Dementia:0.38), (UMECLIDINIUM BROMIDE:Dementia:0.02), (UMECLIDINIUM BROMIDE\VILANTEROL TRIFENATATE:Dementia:0.94), (VALGANCICLOVIR HYDROCHLORIDE:Dementia:0.48), (VITAMIN A:Dementia:0.43), (ZAFIRLUKAST:Dementia:0.05), (ZIPRASIDONE HYDROCHLORIDE\ZIPRASIDONE MESYLATE:Dementia:0.23), (ACETAMINOPHEN\HYDROCODONE:Dementia Alzheimer's type:0.33), (ACETAMINOPHEN\OXYCODONE HYDROCHLORIDE\OXYCODONE TEREPHTHALATE:Dementia Alzheimer's type:0.01), (ACETAMINOPHEN\OXYCODONE\OXYCODONE HYDROCHLORIDE:Dementia Alzheimer's type:0.47), (ADENINE:Dementia Alzheimer's type:1.18), (AGALSIDASE BETA:Dementia Alzheimer's type:0.27), (ALCOHOL:Dementia Alzheimer's type:0.48), (ALISKIREN HEMIFUMARATE:Dementia Alzheimer's type:0.07), (ALOGLIPTIN:Dementia Alzheimer's type:0), (AMISULPRIDE:Dementia Alzheimer's type:1.3), (AMLODIPINE BESYLATE\HYDROCHLOROTHIAZIDE\OLMESARTAN MEDOXOMIL:Dementia Alzheimer's type:0.09), (AMLODIPINE BESYLATE\HYDROCHLOROTHIAZIDE\VALSARTAN:Dementia Alzheimer's type:0.55), (AMOXAPINE:Dementia Alzheimer's type:1.07), (ASPIRIN\LANSOPRAZOLE:Dementia Alzheimer's type:0.5), (BENAZEPRIL\BENAZEPRIL HYDROCHLORIDE:Dementia Alzheimer's type:0.45), (BENZBROMARONE:Dementia Alzheimer's type:0.08), (BENZONATATE:Dementia Alzheimer's type:0.29), (BETAMETHASONE SODIUM PHOSPHATE\NEOMYCIN SULFATE:Dementia Alzheimer's type:0.01), (BETHANECHOL CHLORIDE:Dementia Alzheimer's type:0.21), (BILASTINE:Dementia Alzheimer's type:0.21), (BIXALOMER:Dementia Alzheimer's type:0.06), (BOCOCIZUMAB:Dementia Alzheimer's type:0.08), (BUCILLAMINE:Dementia Alzheimer's type:0.13), (BUPRENORPHINE HYDROCHLORIDE:Dementia Alzheimer's type:0.86), (BUPRENORPHINE HYDROCHLORIDE\NALOXONE HYDROCHLORIDE:Dementia Alzheimer's type:0.06), (BUTYLSCOPOLAMINE BROMIDE:Dementia Alzheimer's type:0.34), (CALCIUM ACETATE:Dementia Alzheimer's type:1.04), (CALCIUM CARBONATE\VITAMIN D:Dementia Alzheimer's type:0.12), (CALCIUM CHLORIDE\POTASSIUM CHLORIDE\SODIUM CHLORIDE\SODIUM LACTATE\SORBITOL:Dementia Alzheimer's type:0), (CALCIUM LACTATE:Dementia Alzheimer's type:0.36), (CANAGLIFLOZIN\METFORMIN HYDROCHLORIDE:Dementia Alzheimer's type:0.48), (CARVEDILOL PHOSPHATE:Dementia Alzheimer's type:1.38), (CEFDITOREN PIVOXIL:Dementia Alzheimer's type:0.3), (CHLORPROMAZINE\PROMETHAZINE:Dementia Alzheimer's type:0.24), (CLOTRIMAZOLE:Dementia Alzheimer's type:0.83), (COLESTILAN CHLORIDE:Dementia Alzheimer's type:0.09), (CORTICOTROPIN:Dementia Alzheimer's type:0.06), (CYANAMIDE:Dementia Alzheimer's type:0), (DALFAMPRIDINE:Dementia Alzheimer's type:0.17), (DANAZOL:Dementia Alzheimer's type:0.2), (DAPAGLIFLOZIN PROPANEDIOL:Dementia Alzheimer's type:0.9), (DEGARELIX:Dementia Alzheimer's type:0.26), (DEXAMETHASONE ACETATE:Dementia Alzheimer's type:0.46), (DEXTROAMPHETAMINE SULFATE:Dementia Alzheimer's type:0.02), (DILAZEP:Dementia Alzheimer's type:0.34), (DOFETILIDE:Dementia Alzheimer's type:0.99), (DOXEPIN HYDROCHLORIDE:Dementia Alzheimer's type:0.45), (DOXYCYCLINE:Dementia Alzheimer's type:0.44), (DUTASTERIDE\TAMSULOSIN HYDROCHLORIDE:Dementia Alzheimer's type:0.37), (ECONAZOLE:Dementia Alzheimer's type:0.06), (EDARAVONE:Dementia Alzheimer's type:0.21), (ELETRIPTAN HYDROBROMIDE:Dementia Alzheimer's type:0.78), (EMPAGLIFLOZIN\LINAGLIPTIN:Dementia Alzheimer's type:0.22), (ENALAPRIL MALEATE\HYDROCHLOROTHIAZIDE:Dementia Alzheimer's type:0.32), (ETORICOXIB:Dementia Alzheimer's type:0.98), (ETRETINATE:Dementia Alzheimer's type:0.82), (EVEROLIMUS:Dementia Alzheimer's type:1.44), (EVOLOCUMAB:Dementia Alzheimer's type:1.08), (FENOTEROL HYDROBROMIDE\IPRATROPIUM BROMIDE:Dementia Alzheimer's type:0.43), (FENTANYL CITRATE:Dementia Alzheimer's type:0.32), (FERRIC CITRATE ANHYDROUS:Dementia Alzheimer's type:0.47), (FERRIC HYDROXIDE:Dementia Alzheimer's type:0.38), (FERRIC OXYHYDROXIDE:Dementia Alzheimer's type:0.06), (FERROUS GLYCINE SULFATE:Dementia Alzheimer's type:0.09), (FERROUS SULFATE\GLYCINE:Dementia Alzheimer's type:0.17), (FEXOFENADINE\PSEUDOEPHEDRINE:Dementia Alzheimer's type:0.16), (FLUTICASONE FUROATE:Dementia Alzheimer's type:0.48), (FORMOTEROL:Dementia Alzheimer's type:0.1), (FORMOTEROL FUMARATE DIHYDRATE\MOMETASONE FUROATE:Dementia Alzheimer's type:0.28), (FORMOTEROL FUMARATE\MOMETASONE FUROATE:Dementia Alzheimer's type:0.05), (FUSIDIC ACID:Dementia Alzheimer's type:0.21), (GLUTAMINE\SODIUM GUALENATE:Dementia Alzheimer's type:0.08), (GLYCOPYRROLATE\INDACATEROL MALEATE:Dementia Alzheimer's type:0.1), (GLYCOPYRRONIUM:Dementia Alzheimer's type:0.04), (GUANFACINE:Dementia Alzheimer's type:0.37), (GUANFACINE HYDROCHLORIDE:Dementia Alzheimer's type:0.01), (HYDROXYCHLOROQUINE DIPHOSPHATE:Dementia Alzheimer's type:0.08), (HYOSCYAMINE:Dementia Alzheimer's type:0.08), (IGURATIMOD:Dementia Alzheimer's type:0.05), (ILOPROST:Dementia Alzheimer's type:0.16), (IMATINIB MESYLATE:Dementia Alzheimer's type:0.84), (IMIPRAMINE HYDROCHLORIDE:Dementia Alzheimer's type:0.65), (INSULIN ASPART\INSULIN DEGLUDEC:Dementia Alzheimer's type:0.61), (IODINE:Dementia Alzheimer's type:0.03), (ISTRADEFYLLINE:Dementia Alzheimer's type:0.23), (LEDIPASVIR\SOFOSBUVIR:Dementia Alzheimer's type:1.31), (LEVALBUTEROL HYDROCHLORIDE:Dementia Alzheimer's type:0.73), (LEVOCABASTINE HYDROCHLORIDE:Dementia Alzheimer's type:0.38), (LEVOCARNITINE:Dementia Alzheimer's type:0.12), (LOXOPROFEN SODIUM DIHYDRATE:Dementia Alzheimer's type:0.13), (MAXACALCITOL:Dementia Alzheimer's type:0.41), (MEPERIDINE:Dementia Alzheimer's type:0.36), (MEPOLIZUMAB:Dementia Alzheimer's type:0.01), (METAXALONE:Dementia Alzheimer's type:0.68), (METHENAMINE HIPPURATE:Dementia Alzheimer's type:0.33), (METHYLPREDNISOLONE ACETATE:Dementia Alzheimer's type:0.81), (MOEXIPRIL HYDROCHLORIDE:Dementia Alzheimer's type:0.24), (MOXIFLOXACIN HYDROCHLORIDE:Dementia Alzheimer's type:1.41), (MYCOPHENOLATE MOFETIL\MYCOPHENOLATE MOFETIL HYDROCHLORIDE:Dementia Alzheimer's type:0.64), (NADOLOL:Dementia Alzheimer's type:0.6), (NADROPARIN CALCIUM:Dementia Alzheimer's type:0.52), (NALOXONE HYDROCHLORIDE\TILIDINE HYDROCHLORIDE:Dementia Alzheimer's type:0.61), (OLODATEROL HYDROCHLORIDE\TIOTROPIUM BROMIDE MONOHYDRATE:Dementia Alzheimer's type:0.91), (OMEPRAZOLE SODIUM:Dementia Alzheimer's type:0.61), (ONABOTULINUMTOXINA:Dementia Alzheimer's type:0.14), (OXYBUTYNIN\OXYBUTYNIN CHLORIDE:Dementia Alzheimer's type:0.12), (PALIPERIDONE PALMITATE:Dementia Alzheimer's type:0.57), (PATIROMER:Dementia Alzheimer's type:0.19), (PEGINTERFERON BETA-1A:Dementia Alzheimer's type:0.24), (PEGZEREPOETIN ALFA:Dementia Alzheimer's type:0.08), (PEMETREXED DISODIUM:Dementia Alzheimer's type:0.57), (PENICILLIN:Dementia Alzheimer's type:0.74), (PHENOTHIAZINE:Dementia Alzheimer's type:0.2), (PHYTONADIONE:Dementia Alzheimer's type:0.04), (PIMOBENDAN:Dementia Alzheimer's type:0.92), (PIROXICAM:Dementia Alzheimer's type:0.36), (POLYSTYRENE:Dementia Alzheimer's type:0.42), (POLYSTYRENE SULFONIC ACID:Dementia Alzheimer's type:0.01), (PONATINIB:Dementia Alzheimer's type:0.1), (POTASSIUM CITRATE\TRISODIUM CITRATE DIHYDRATE:Dementia Alzheimer's type:0.46), (PREDNISOLONE ACETATE:Dementia Alzheimer's type:0.2), (PROCATEROL HYDROCHLORIDE:Dementia Alzheimer's type:0.07), (PROCHLORPERAZINE MALEATE:Dementia Alzheimer's type:0.14), (PROCYCLIDINE:Dementia Alzheimer's type:0.04), (PYRIDOSTIGMINE BROMIDE:Dementia Alzheimer's type:0.06), (PYRIDOXINE:Dementia Alzheimer's type:0.23), (QUAZEPAM:Dementia Alzheimer's type:0.27), (RASAGILINE:Dementia Alzheimer's type:0.07), (RILMENIDINE PHOSPHATE:Dementia Alzheimer's type:0.08), (RIZATRIPTAN BENZOATE:Dementia Alzheimer's type:0.18), (ROFLUMILAST:Dementia Alzheimer's type:0.72), (SACUBITRIL\VALSARTAN:Dementia Alzheimer's type:1.33), (SALMETEROL XINAFOATE:Dementia Alzheimer's type:0.08), (SARPOGRELATE HYDROCHLORIDE:Dementia Alzheimer's type:0.57), (SCOPOLAMINE:Dementia Alzheimer's type:1.23), (SECUKINUMAB:Dementia Alzheimer's type:0.41), (SUMATRIPTAN:Dementia Alzheimer's type:0.2), (SUMATRIPTAN\SUMATRIPTAN SUCCINATE:Dementia Alzheimer's type:0.28), (SUNITINIB MALATE:Dementia Alzheimer's type:0.29), (SUPLATAST TOSILATE:Dementia Alzheimer's type:0.42), (TESTOSTERONE UNDECANOATE:Dementia Alzheimer's type:0.11), (TETRACYCLINE:Dementia Alzheimer's type:0.3), (THYROID, PORCINE:Dementia Alzheimer's type:0.32), (TIZANIDINE HYDROCHLORIDE:Dementia Alzheimer's type:1.22), (TOCILIZUMAB:Dementia Alzheimer's type:0.11), (TRIHEXYPHENIDYL:Dementia Alzheimer's type:0.43), (TRIMIPRAMINE MALEATE:Dementia Alzheimer's type:0.44), (VARENICLINE TARTRATE:Dementia Alzheimer's type:1.78), (ACETAMINOPHEN\CODEINE PHOSPHATE:Dementia with Lewy bodies:0.06), (AMITRIPTYLINE:Dementia with Lewy bodies:0.08), (BISOPROLOL FUMARATE:Dementia with Lewy bodies:0.16), (DEXLANSOPRAZOLE:Dementia with Lewy bodies:0.66), (DILTIAZEM:Dementia with Lewy bodies:0.17), (DILTIAZEM HYDROCHLORIDE:Dementia with Lewy bodies:0.2), (IBRUTINIB:Dementia with Lewy bodies:0.24), (LITHIUM CARBONATE:Dementia with Lewy bodies:0.04), (SACUBITRIL\VALSARTAN:Dementia with Lewy bodies:0.08), (VARENICLINE TARTRATE:Dementia with Lewy bodies:0.09), (BISOPROLOL FUMARATE:Dental care:0.02), (BORTEZOMIB:Dental care:0), (BUPROPION HYDROCHLORIDE:Dental care:0.84), (CALCIUM CARBONATE\CHOLECALCIFEROL:Dental care:0.59), (CERTOLIZUMAB PEGOL:Dental care:0.89), (CHOLESTYRAMINE:Dental care:0.02), (CLOTRIMAZOLE:Dental care:0.07), (DIVALPROEX SODIUM:Dental care:0.15), (DULOXETINE:Dental care:0.06), (ESZOPICLONE:Dental care:0.03), (EVOLOCUMAB:Dental care:0.31), (FINGOLIMOD HYDROCHLORIDE:Dental care:0.13), (IBRUTINIB:Dental care:1.74), (ILOPROST:Dental care:0.33), (METAXALONE:Dental care:0.01), (METHYLPREDNISOLONE SODIUM SUCCINATE:Dental care:0.29), (NEBIVOLOL:Dental care:0.43), (OLANZAPINE:Dental care:0.09), (PANTOPRAZOLE:Dental care:0.7), (PHENOBARBITAL:Dental care:0.01), (PRAVASTATIN:Dental care:0.38), (PROMETHAZINE\PROMETHAZINE HYDROCHLORIDE:Dental care:0.15), (RANOLAZINE:Dental care:0.32), (SACUBITRIL\VALSARTAN:Dental care:0.38), (TERIFLUNOMIDE:Dental care:0.41), (TOPIRAMATE:Dental care:0.92), (ACETAMINOPHEN\CODEINE PHOSPHATE:Dental disorder prophylaxis:0.12), (ANASTROZOLE:Dental disorder prophylaxis:0.11), (CHOLESTYRAMINE:Dental disorder prophylaxis:0.02), (DASATINIB:Dental disorder prophylaxis:0.02), (DEXLANSOPRAZOLE:Dental disorder prophylaxis:0.09), (DIVALPROEX SODIUM:Dental disorder prophylaxis:0.45), (ENALAPRIL:Dental disorder prophylaxis:0.43), (FERROUS SULFATE\FERROUS SULFATE, DRIED:Dental disorder prophylaxis:0.5), (IBRUTINIB:Dental disorder prophylaxis:1.04), (ILOPROST:Dental disorder prophylaxis:0.11), (INSULIN ASPART:Dental disorder prophylaxis:0), (METOPROLOL TARTRATE:Dental disorder prophylaxis:0.02), (NAPROXEN:Dental disorder prophylaxis:0.38), (NEBIVOLOL:Dental disorder prophylaxis:0.06), (NILOTINIB:Dental disorder prophylaxis:0.37), (PAROXETINE:Dental disorder prophylaxis:0.09), (PROMETHAZINE\PROMETHAZINE HYDROCHLORIDE:Dental disorder prophylaxis:0.08), (TAMSULOSIN:Dental disorder prophylaxis:0.5), (THALIDOMIDE:Dental disorder prophylaxis:0.76), (VARENICLINE TARTRATE:Dental disorder prophylaxis:0.12), (ACETAMINOPHEN\BUTALBITAL\CAFFEINE:Depressed mood:0.26), (ANASTROZOLE:Depressed mood:1.06), (BENZTROPINE:Depressed mood:0.04), (BENZTROPINE MESYLATE:Depressed mood:0.02), (BISACODYL OR DOCUSATE SODIUM:Depressed mood:0.37), (BUDESONIDE:Depressed mood:1.1), (BUPRENORPHINE HYDROCHLORIDE:Depressed mood:0.89), (CALCITRIOL:Depressed mood:0.12), (CALCIUM ACETATE:Depressed mood:0.02), (CEFTRIAXONE SODIUM:Depressed mood:0.08), (CERTOLIZUMAB PEGOL:Depressed mood:0.54), (CHOLESTYRAMINE:Depressed mood:0.37), (CLINDAMYCIN\CLINDAMYCIN PHOSPHATE:Depressed mood:0.66), (COLCHICINE:Depressed mood:0.34), (DAPAGLIFLOZIN PROPANEDIOL:Depressed mood:0.11), (DASATINIB:Depressed mood:0.37), (DICYCLOMINE HYDROCHLORIDE:Depressed mood:0.05), (DIPHENHYDRAMINE:Depressed mood:0.73), (DOXAZOSIN MESYLATE:Depressed mood:0.15), (DRONABINOL:Depressed mood:0.22), (DUTASTERIDE:Depressed mood:0.47), (ESTROGENS, CONJUGATED\MEDROXYPROGESTERONE ACETATE:Depressed mood:0.05), (EVOLOCUMAB:Depressed mood:0.77), (FLUTICASONE:Depressed mood:0.1), (GUANFACINE HYDROCHLORIDE:Depressed mood:0.04), (HYDROCHLOROTHIAZIDE\IRBESARTAN:Depressed mood:0.15), (HYDROCHLOROTHIAZIDE\LISINOPRIL:Depressed mood:0.69), (HYDROMORPHONE HYDROCHLORIDE:Depressed mood:0.7), (IMATINIB MESYLATE:Depressed mood:0.55), (IMIPRAMINE HYDROCHLORIDE:Depressed mood:0.02), (INDAPAMIDE\PERINDOPRIL:Depressed mood:0.44), (INDOMETHACIN:Depressed mood:0.18), (IRBESARTAN:Depressed mood:0.52), (LABETALOL\LABETALOL HYDROCHLORIDE:Depressed mood:0.33), (LERCANIDIPINE HYDROCHLORIDE:Depressed mood:0.4), (METAMIZOLE:Depressed mood:0.04), (METAXALONE:Depressed mood:0.26), (METHADONE HYDROCHLORIDE:Depressed mood:0.47), (METHOCARBAMOL:Depressed mood:0.36), (METHYLPREDNISOLONE SODIUM SUCCINATE:Depressed mood:1.1), (MOXIFLOXACIN HYDROCHLORIDE:Depressed mood:0.67), (NALTREXONE:Depressed mood:0.07), (NAPROXEN SODIUM:Depressed mood:0.42), (NEBIVOLOL HYDROCHLORIDE:Depressed mood:0.24), (OMEGA-3-ACID ETHYL ESTERS:Depressed mood:0.15), (OXYGEN:Depressed mood:0.38), (PANTOPRAZOLE MAGNESIUM:Depressed mood:0.18), (PITAVASTATIN CALCIUM:Depressed mood:0), (PRIMIDONE:Depressed mood:0.64), (PROGESTERONE:Depressed mood:0.1), (PYRIDOXINE HYDROCHLORIDE:Depressed mood:0.07), (RABEPRAZOLE SODIUM:Depressed mood:0.21), (RANOLAZINE:Depressed mood:0.79), (RIZATRIPTAN BENZOATE:Depressed mood:0.32), (ROPINIROLE HYDROCHLORIDE:Depressed mood:0.49), (SODIUM OXYBATE:Depressed mood:1.15), (TOCILIZUMAB:Depressed mood:0.2), (VALACYCLOVIR HYDROCHLORIDE:Depressed mood:0.33), (VERAPAMIL HYDROCHLORIDE:Depressed mood:1.03), (VITAMIN B:Depressed mood:0.34), (ZONISAMIDE:Depressed mood:0.48), (4-HYDROXYBUTANOIC ACID:Depression:0.08), (ABACAVIR\LAMIVUDINE\ZIDOVUDINE:Depression:0.14), (ABA LOPARATIDE:Depression:0.57), (ACETAMINOPHEN\ASPIRIN\SALICYLAMIDE:Depression: 0.48), (ACETAMINOPHEN\BROMISOVAL\DIHYDROCODEINE PHOSPHATE\DIPHENHYDRAMINE SALICYLATE\DYPHYLLINE\METHYLEPHEDRINE HYDROCHLORIDE, (+/−)-:Depression:0.01), (ACETAMINOPHEN\BUTYLSCOPOLAMINE BROMIDE:Depression:0.63), (ACETAMINOPHEN\CAFFEINE\DIHYDROCODEINE BITARTRATE:Depression:0.28), (ACETAMINOPHEN\CHLORPHENIRAMINE MALEATE\DEXTROMETHORPHAN HYDROBROMIDE\PSEUDOEPHEDRINE HYDROCHLORIDE:Depression:0.34), (ACETAMINOPHEN\CHLORPHENIRAMINE MALEATE\PHENYLEPHRINE HYDROCHLORIDE OR CETIRIZINE HYDROCHLORIDE OR CHLORPHENIRAMINE MALEATE OR CLEMASTINE FUMARATE OR DIPHENHYDRAMINE HYDROCHLORIDE OR FEXOFENADINE HYDROCHLORIDE OR LORATADINE:Depression:0.74), (ACETAMINOPHEN\OPIUM:Depression:0.77), (ACETAMI NOPHEN\PHENYLEPHRINE HYDROCHLORIDE:Depression:0.3), (ACETAMINOPHEN\PSEUDOEPHEDRINE:Depression: 0.14), (ACETIC ACID\SODIUM ACETATE:Depression:0.55), (ACETOHYDROXAMIC ACID:Depression:0.69), (ACLIDINIUM BROMIDE\FORMOTEROL FUMARATE:Depression:0.2), (ACRIVASTINE:Depression:0.55), (ACTIVATED CHARCOAL:Depression:2.02), (ADEFOVIR:Depression:0.12), (ADENINE:Depression:1.08), (A LBUTEROL\GUAIFENESIN:Depression:0.77), (ALBUTEROL\IPRATROPIUM BROMIDE:Depression:0.87), (ALDIOXA:Depression:0.14), (ALDOSTERONE:Depression:0.07), (ALFALFA\ALFALFA WHOLE:Depression:0.12), (ALISKIREN HEMIFUMARATE\VALSARTAN:Depression:1.08), (ALISKIREN\HYDROCHLOROTHIAZI DE:Depression:0.93), (ALOSETRON:Depression:0.45), (ALPHA-TOCOPHEROL\ASCORBIC ACID\CHOLECALCIFEROL\COPPER\CYANOCOBALAMIN\FOLIC ACID\LUTEIN\OMEGA-3 FATTY ACIDS\PYRIDOXINE HYDROCHLORIDE\TAURINE\VITAMIN A PALMITATE\ZEAXANTHIN\ZINC PICOLINATE:Depression:0.27), (ALPHA-TOCOPHEROL\CHOLECALCIFEROL:Depression:0.23), (ALPROSTADIL\PAPAVERINE\P HENTOLAMINE:Depression:0.22), (ALUMINUM ACETATE:Depression:0.34), (ALUMINUM HYDROXIDE\DIMETHICONE\DIPHENHYDRAMINE\HYDROXIDE ION\LIDOCAINE:Depression:0.11), (ALUMINUM HYDROXIDE\MAGNESIUM HYDROXIDE\MAGNESIUM TRISILICATE:Depression:0.03), (ALUMINUM HYDROXIDE\MAGNESIUM TRISILICATE:Depression:0), (ALUMINUM SILICATE:Depression:0.04), (AMINOPYRINE\ANTIPYRINE\CAFFEINE\PHENACETIN: Depression:0.99), (AMLODIPINE BESYLATE\ATORVASTATIN CALCIUM TRIHYDRATE:Depression:0.14), (AMLODIPINE\ATORVASTATIN:Depression:0.05), (AMM ONIUM GLYCYRRHIZATE\GLYCINE\METHIONINE:Depression:0.58), (AMOBARBITAL SODIUM:Depression:1.66), (AMPHETAMINE ADIPATE\DEXTROAMPHETAMINE:Depression:0.64), (AMPHETAMINE ASPARTATE MONOHYDRATE\AMPHETAMINE SULFATE\DEXTROAMPHETAMINE SACCHARATE\DEXTROAMPHETAMINE SULFATE:Depression:0.96), (AMPHETAMINE ASPARTATE\DEXTROAMPHETAMINE SACCHARATE:Depression:0.91), (AMPICILLIN TRIHYDRATE:Depression:0.03), (ANAGLIPTIN:Depression:0.79), (ANGIOTENSIN II:Depression:0.07), (ANTI-INHIBITOR COAGULANT COMPLEX:Depression:0.07), (ARABICA COFFEE BEAN\COFFEE BEAN:Depression:0.32), (ARCTOSTAPHYLOS UVA-URSI LEAF\URTICA DIOICA:Depression:0.93), (ARFORMOTEROL:Depression:0.48), (ARTEMETHER\LUMEFAN TRINE:Depression:0.39), (ARTICAINE HYDROCHLORIDE\EPINEPHRINE BITARTRATE:Depression:0.11), (ASCORBIC ACID\CHONDROITIN SULFATE (BOVINE)\GLUCOSAMINE SULFATE\MANGANESE:Depression:0.5), (ASCORBIC ACID\FERROUS ASPARTO GLYCINATE\FERROUS FUMARATE\FOLIC ACID\NIACIN:Depression:0.19), (ASPARTAME:Depression:0.55), (ASPIRIN\CAFFEINE\SAL ICYLAMIDE:Depression:1.14), (ASPIRIN\MAGNESIUM HYDROXIDE:Depression:0.2), (ATRACURIUM BESYLATE:Depression:0.42), (ATROPINE\DIPHENOXYLATE HYDROCHLORIDE:Depression:0.35), (AVOBENZONE\HOMOSALATE\OCTISALATE: Depression:1.58), (AZITHROMYCIN MONOHYDRATE:Depression:2.16), (BACILLUS SUBTILIS:Depression:0.1), (BACITRACIN ZINC:Depression:1.01), (BACITRACIN\NEOMYCIN SULFATE\POLYMYXIN B SULFATE:Depression:0.2), (BACLOFEN\CYCLOBENZAPRINE\DICLOFENAC\GABAPEN TIN\TETRACAINE:Depression:0.98), (BALSAM PERU OIL\CASTOR OIL\TRYPSIN:Depression:0.19), (BELATACEPT:Depression:0.93), (BENFOTIAMINE:Depression: 1.02), (BENZOCAINE\RESORCINOL:Depression:0.52), (BENZPHETAMINE HYDROCHLORIDE:Depression:0.16), (BEPOTASTINE\BEPOTASTINE BESILATE:Depression:0.34), (BEPRIDIL:Depression:1.71), (BEPRIDIL HYDROCHLORIDE:Depression:0.48), (BETA GLUCAN:Depression:0.85), (BETAMETHASONE BUTYRATE PROPIONATE:Depression:0.44), (BETAMETHASONE DIPROPIONATE\CALCIPOTRIENE HYDRATE:Depression:1.23), (BETAMETHASONE DIPROPIONATE\CLOTRIMAZOLE\GENTAMICIN SULFATE:Depression:0.37), (BETAMETHASONE DIPROPIONATE\GENTAMICIN SULFATE:Depression:0.36), (BIFIDOBACTERIUM LONGUM:Depression:0.26), (BIOFLAVONOIDS:Depression:0.01), (BIOTIN\CALCIUM PANTOTHENATE\CHOLINE BITARTRATE\CYANOCOBALAMIN\FOLIC ACID\INOSITOL\NIACINAMIDE\PYRIDOXINE\RIBOFLAVIN\THIAMINE:Depression:0.43), (BIPERIDEN LACTATE:Depression:1.9), (BISMUTH:Depression:0.06), (BISMUTH SUBSALICYLATE\GLYCERIN\PROPYLENE GLYCOL:Depression:0.19), (BIXALOMER:Depression:0.56), (BORNAPRINE HYDROCHLORIDE:Depression:1.2), (BOTULINUM TOXIN NOS:Depression:0.42), (BRIMONIDINE TARTRATE\DORZOLAMIDE HYDROCHLORIDE\TIMOLOL MALEATE:Depression:0.24), (BROMPHENIRAMINE:Depression:0.01), (BROMPHENIRAMI NE MALEATE\DEXTROMETHORPHAN HYDROBROMIDE\PSEUDOEPHEDRINE HYDROCHLORIDE:Depression:0.71), (BROMPHENIRAMINE MALEATE\PSEUDOEPHEDRINE HYDROCHLORIDE:Depression:0.59), (BUCOLOME:Depression:0.04), (BUFORMIN:Depression: 0.4), (BUTAMIRATE CITRATE:Depression:0.17), (BUTYLSCOPOLAMINE BROMIDE\METAMIZOLE SODIUM:Depression:0.2), (CAFFEINE\ERGOTAMINE TARTRATE:Depression:0.84), (CAFFEINE\INDOMETHACIN\PROCHLORPERAZINE MALEATE:Depression:0.22), (CAFFEINE\ISOMETHEPTENE\METHIMAZOLE:Depression:10.09), (CAJUPUT OIL\EUCALYPTUS OIL\PEPPERMINT OIL:Depression:1.73), (CALCIUM ASCORBATE:Depression:1.19), (CALCIUM CHLORIDE\DEXTROSE MONOHYDRATE\MAGNESIUM CHLORIDE\SODIUM CHLORIDE\SODIUM LACTATE:Depression:0.11), (CALCIUM CHLORIDE\MAGNESIUM CHLORIDE\POTASSIUM CHLORIDE\POTASSIUM PHOSPHATE, DIBASIC\POTASSIUM PHOSPHATE, MONOBASIC\SODIUM CHLORIDE:Depression:0.37), (CALCIUM CHLORIDE\POTASSIUM CHLORIDE\SODIUM CHLORIDE\SODIUM LACTATE\SORBITOL:Depression:0.61), (CALCIUM DOBESILATE:Depression:0.1), (CALCIUM PANTOTHENATE\NIACINAMIDE\PYRIDOXINE HYDROCHLORIDE\RIBOFLAVIN\THIAMINE HYDROCHLORIDE:Depression:0.46), (CAMPHOR\MENTHOL\METHYL SALICYLATE:Depression:0.33), (CANDESARTAN CILEXETIL\FELODIPINE:Depression:0.16), (CANNABIS SATIVA SEED OIL:Depression:0.75), (CAPSICUM:Depression:1), (CAPTOPRIL\HYDROCHLOROTHIAZID E:Depression:0.55), (CARBAZOCHROME SODIUM SULFONATE:Depression:0.86), (CARBOPROST TROMETHAMINE:Depression:0.06), (CARBOXYMETHYLCELLULOSE SODIUM\HYPROMELLOSES:Depression:0.2), (CARBOXYMETHYLCELLULOSE SODIUM\SORBINICATE:Depression:0.67), (CARMUSTINE:Depression:0.7), (CEFATRIZINE:Depression:0.05), (CEFCAPENE PIVOXIL:Depression:0.02), (CEFTERAM PIVOXIL:Depression:0.27), (CEPHALEXIN HYDROCHLORIDE:Depression:0.3), (CEPHRADINE:Depression:0.83), (CERIVASTATIN: Depression:0.94), (CHAMOMILE:Depression:1.28), (CHIMAPHILA UMBELLATA\MANGANESE CHLORIDE\POPULUS TREMULOIDES\SODIUM TAUROCHOLATE:Depression:0.2), (CHLOPHEDIANOL HYDROCHLORIDE\DEXBROMPHENIRAMINE MALEATE\PHENYLEPHRINE HYDROCHLORIDE:Depression:0.68), (CHLORAMPHENICOL\PREDNISOLONE:Depression: 0.65), (CHLORHEXIDINE GLUCONATE\LIDOCAINE HYDROCHLORIDE:Depression:0.19), (CHLORIDE ION:Depression:0.58), (CHLORMADINONE ACETATE\ETHINYL ESTRADIOL:Depression:1.3), (CHLORPHENIRAMINE MALEAT\DIHYDROCODEINE PHOSPHATE\METHYLEPHEDRINE HYDROCHLORIDE, DL-:Depression:0.19), (CHLORPHENIRAMINE MALEATE\HYDROCODONE BITARTRATE\PSEUDOEPHEDRINE HYDROCHLORIDE:Depression:0.3), (CHLORPHENIRAMINE MALEATEIBUPROFEN\PSEUDOEPHEDRINE HYDROCHLORIDE:Depression:0), (CHLORPHENIRAMINE MALEATE\PHENYLEPHRINE HYDROCHLORIDE:Depression:0.47), (CHLORPHENIRAMINE MALEATE\PSEUDOEPHEDRINE HYDROCHLORIDE:Depression:0.41), (CHLORPROMAZINE HIBENZATE:Depression:0.35), (CHOLECALCIFEROL\GENISTEIN\ZINC GLYCINATE:Depression:0.87), (CHOLINE BITARTRATE:Depression:0.44), (CHOLINE SALICYLATE:Depression:0.1), (CHOLINETRAZODONE HYDROCHLORIDE:Depression:0.69), (CICLOPIROX OLAMINE:Depression:1.55), (CIFENLINE SUCCINATE:Depression:0.68), (CINNAMON OIL:Depression:0.55), (CIPROFLOXACIN HYDROCHLORIDE\HYDROCORTISONE:Depression:0.54), (CIPROFLOXACIN\DEXAMET HASONE:Depression:0.53), (CITRIC ACID MONOHYDRATE\MAGNESIUM OXIDE\SODIUM PICOSULFATE:Depression:0.18), (CITRIC ACID MONOHYDRATE\POTASSIUM CITRATE:Depression:0.62), (CITRIC ACID MONOHYDRATE\TRISODIUM CITRATE DIHYDRATE:Depression:0.51), (CLADRIBINE:Depression:0.89), (CLARITHROMYCIN LACTOBIONATE:Depression:0.22), (CLOBETASONE BUTYRATE\NYSTATIN\OXYTETRACYCLINE CALCIUM:Depression:0.27), (CLOPENTHIXOL HYDROCHLORIDE:Depression:0.68), (CLOPERASTINE:Depression:0.45), (CLOPREDNOL: Depression:0.52), (CLORAZEPIC ACID:Depression:0.39), (CLOSTRIDIUM TETANI\CLOSTRIDIUM TETANI TOXOID ANTIGEN (FORMALDEHYDE INACTIVATED)\CORYNEBACTERIUM DIPHTHERIAE\CORYNEBACTERIUM DIPHTHERIAE TOXOID ANTIGEN (FORMALDEHYDE INACTIVATED):Depression:0.86), (COAGULATION FACTOR VIIA RECOMBINANT HUMAN:Depression:0.25), (COBAMAMIDE:Depression:0.27), (CODEINE PHOSPHATE\GUAIFENESIN\PHENIRAMINE MALEATE:Depression:0.14), (CODEINE PHOSPHATE\PROMETHAZINE:Depression:0.9), (COLESTILAN CHLORIDE:Depression:0.76), (COPPER GLUCONATE:Depression:0.13), (CORTIVAZOL:Depression:0.22), (COSYNTROPIN:Depression: 0.57), (CRATAEGUS LAEVIGATA FRUIT:Depression:0.34), (CROFELEMER:Depression:0.5), (CUMIN:Depression:0.36), (CYAN OCOBALAMIN\LIDOCAINE HYDROCHLORIDE\PYRIDOXINE HYDROCHLORIDE\THIAMINE HYDROCHLORIDE:Depression:0.31), (CYSTEAMINE BITARTRATE:Depression:0.2), (CYSTINE:Depression:0.75), (DACARBAZINE:Depression:0.29), (DASABUVIR SODIUM MONOHYDRATE:Depression:0.21), (DEFEROXAMINE MESYLATE:Depression:1.33), (DEGARELIX ACETATE:Depression:1.38), (DEMECLOCYCLINE:Depression:0.51), (DEOXYRIBONUCLIE C ACID:Depression:0.3), (DEPRODONE PROPIONATE:Depression:0.07), (DEQUALINIUM CHLORIDE:Depression:0.8), (DESLORATADINE\PSEUDOEPHEDRINE SULFATE:Depression:0.95), (DEXAMETHASONEFRAMYCETIN SULFATE\GRAMICIDIN:Depression:0.88), (DEXTROMETHORPHAN HYDROBROMIDE\GUAIFENESIN\PHENYLEPHRINE HYDROCHLORIDE:Depression:0.5), (DEXTROMETHORPHAN HYDROBROMIDE\GUAIFENESIN\PSEUDOEPHEDRINE HYDROCHLORIDE:Depression:0.83), (DEXTROSE MONOHYDRATE\SODIUM CHLORIDE:Depression:0.08), (DEXTROSE\FRUCTOSE\PHOSPHORIC ACID:Depression:0.07), (DIACETYL BENZOYL LATHYROL:Depression:0), (DIASTASE:Depression:0.52), (DICLOXACILLIN SODIUM:Depression:0.12), (DIETARY SUPPLEMENT\FLAVOCOXID:Depression:0.86), (DIETARY SUPPLEMENT\RESVERATROL:Depression:1.38), (DIFLUNISAL:Depression:2.36), (DIGITA LIS:Depression:1.15), (DIHYDROERGOCRISTINE:Depression:0.16), (DILAZEP:Depression:1.18), (DIMEMORFAN:Depression:0.36), (DIMETHYL SULFONE\GLUCOSAMINE SULFATE:Depression:0.63), (DIMETHYL SULFOXIDE:Depression:0.87), (DIPHENHYDRAMINE HYDROCHLORIDE\PHENYLEPHRINE HYDROCHLORIDE:Depression:1), (DOCONEXENT\ICOSAPENT\FISH OIL\OMEGA-3 FATTY ACIDS:Depression:0.65), (DOCONEXENT\ICOSAPENT:Depression:0.27), (DOCUSATE SODIUM\SENNOSIDE A\SENNOSIDE B:Depression:1.07), (DOLASETRON MESYLATE:Depression:2.02), (ELECTROLYTES NOS:Depression:0.13), (ELM:Depression:0.33), (ENALAPRIL MALEATE\NITRENDIPINE:Depression:0.3), (EPHEDRA:Depression:0.5), (EPHEDRA SINICA STEM\HERBALS:Depression:0.13), (EPHEDRINE HYDROCHLORIDE:Depression:0.35), (EPHEDRINE HYDROCHLORIDE\GUAIFENESIN:Depression:1.44), (EPINASTINE:Depression:0.3), (ERDO STEINE:Depression:0.2), (ERYTHROMYCIN LACTOBIONATE:Depression:0.74), (ERYTHROMYCIN\SULFISOXAZOLE:Depression:1.81), (ESCHERICHIA COLI:Depression:1.5), (ESMOLOL HYDROCHLORIDE:Depression:1.77), (ESTRADIOL HEMIHYDRATE\NORETHINDRONE ACETATE:Depression:0.85), (ESTRONE:Depression:0.82), (ETEPLIRSEN:Depression:0.28), (E TRETINATE:Depression:1.56), (EZOGABINE:Depression:0.07), (FENOPROFEN CALCIUM:Depression:1.34), (FERRIC POLYSACCHARIDE COMPLEX:Depression:1.19), (FERRIC SULFATE:Depression:0.42), (FERRITIN:Depression:1.76), (FERROUS SULFATE\GASTRIC MUCIN:Depression:0.08), (FEVERFEW:Depression:0.65), (FIMASARTAN:Depression:0.17), (F LAVIN ADENINE DINUCLEOTIDE:Depression:0.96), (FLAVOXATE HYDROCHLORIDE:Depression:0.77), (FLOMOXEF:Depression:0.33), (FLUCYTOSINE: Depression:0.02), (FLUOROURACIL\IRINOTECAN\LEUCOVORIN:Depression:0.43), (FLUOXY MESTERONE:Depression:0.04), (FLUPENTIXOL DIHYDROCHLORIDE:Depression:2.08), (FLUPREDNIDENE ACETATE\MICONAZOLE NITRATE:Depression:0.26), (FLURANDRENOLIDE:Depression:0.61), (FOLIC ACID\IRON POLYMALTOSE:Depression:0.29), (FOLLICLE STIMULATING HORMONE BETA POLYPEPTIDE\LUTROPIN ALFA:Depression:0.22), (FOSFOMYCIN TROMETHAMINE:Depression:0.48), (FUSIDATE SODIUM:Depression:0.56), (GADOBENATE DIMEGLUMINE:Depression:0.79), (GADOLINIUM:Depression:0.86), (GADOTERATE MEGLUMINE:Depression:0.73), (GADOTERIDOL:Depression:0.04), (GADOXETATE DISODIUM:Depression:0.11), (GENISTEIN:Depression:0.85), (GENTIAN VIOLET:Depression:0.7), (GLIMEPIRIDE\PIOGLITAZONE HYDROCHLORIDE:Depression:0.59), (GLIQUIDONE:Depression:0.57), (GLYCINE:Depression: 0.29), (GOLD:Depression:0.35), (GOTU KOLA:Depression:0.74), (GRAPEFRUIT:Depression:0.48), (GRAPEFRUIT JUICE:Depression:0.16), (GRAPEFRUIT SEED EXTRACT:Depression:0.65), (GREEN TEA LEAF:Depression:1.41), (GUANABENZ ACETATE:Depression:0.55), (HALCINONIDE:Depression:1.24), (HALOPERIDOL LACTATE:Depression:1), (HARPAGOPHYTUM PROCUMBENS ROOT:Depression:0.43), (HAWTHORN LEAF WITH FLOWER:Depression:0.24), (HELIUM:Depression:0.35), (HERBS\UNSPECIFIED INGREDIENT:Depression:0.17), (HONEY:Depression:0.03), (HUMAN COLOSTRUM:Depression:0.92), (HYALURONIC ACID\LIDOCAINE HYDROCHLORIDE:Depression:0.01), (HYDROCHLORIC ACID:Depression:0.67), (HYDROCODONE\HYDROCODONE BITARTRATE\IBUPROFEN:Depression:0.24), (HYDROCORTISONE HEMISUCCINATE:Depression:0.49), (HYDROCORTISONE PROBUTATE:Depression:0.07), (HYDROCORTISONE\PRAMOXINE:Depression:0.26), (HYD ROXOCOBALAMIN ACETATE:Depression:0.82), (HYDROXYPROGESTERONE:Depression:0.1), (IBRITUMOMA B TIUXETAN:Depression:1.24), (IBUPROFEN SODIUM:Depression:0.03), (IBUPROFEN\PHENYLEPHRINE HYDROCHLORIDE:Depression:0.09), (IFENPRODIL TARTRATE:Depression:0.09), (IGURATIMOD:Depression:0.39), (INDORAMIN:Depression:0.07), (INFLUENZA A VIRUS A/CALIFORNIA/7/2009 X-179A (H1N1) ANTIGEN (FORMALDEHYDE INACTIVATED)\INFLUENZA A VIRUS A/TEXAS/50/2012 X-223A (H3N2) ANTIGEN (FORMALDEHYDE INACTIVATED)\INFLUENZA B VIRUS B/BRISBANE/60/2008 ANTIGEN (FORMALDEHYDE INACTIVATED)\INFLUENZA B VIRUS B/MASSACHUSETTS/2/2012 ANTIGEN (FORMALDE:Depression:0.19), (INFLUENZA A VIRUS A/CALIFORNIA/7/2009 X-181 (H1N1) ANTIGEN (FORMALDEHYDE INACTIVATED)\INFLUENZA A VIRUS A/VICTORIA/210/2009 X-187 (H3N2) ANTIGEN (FORMALDEHYDE INACTIVATED)\INFLUENZA B VIRUS B/BRISBANE/60/2008 ANTIGEN (FORMALDEHYDE INACTIVATED)\INFLUENZA VIRUS VACCINE:Depression:0.06), (INOSINE PRANOBEX:Depression:0.23), (INSULIN DEGLUDEC\LIRAGLUTIDE:Depression:0.28), (INSULIN GLARGINE\LIXISENATIDE:Depression:0.25), (IOBITRIDOL:Depression:0.03), (IPRAGLIFL OZIN:Depression:0.6), (IPRAGLIFLOZIN L-PROLINE:Depression:1.17), (ISAVUCONAZOLE:Depression:0.4), (ISOFLAVONES SOY:Depression:0.8), (ISOLEUCINE:Depression:0.12), (ISTRADEFYLLINE:Depression:1.97), (ITRACONAZOLE\SODIUM CHLORIDE:Depression:1.45), (IXAZOMIB CITRATE:Depression:0.27), (KETOBEMIDONE:Depression:0.53), (KETOBEMIDONE HYDROCHLORIDE:Depression:0.64), (LACTOBACILLUS ACIDOPHILUS\LACTOBACILLUS RHAMNOSUS:Depression:0.56), (LACTOPEROXIDASE BOVINE\LYSOZYME HYDROCHLORIDE:Depression:1.11), (LANDIOLOL HYDROCHLORIDE:Depression:0.13), (LANOLIN\MINERAL OIL\PETROLATUM:Depression:0.7), (LESINURAD:Depression:0.41), (LETROZOLE\RIBOCI CLIB:Depression:0.55), (LEUPROLIDE:Depression:0.97), (LEVAMLODIPINE:Depression:0.69), (LEVAMLODIPINE BESYLATE:Depression:0.07), (LEVOBUPIVACAINE HYDROCHLORIDE:Depression:0.18), (LEVOMEFOLATE CALCIUM\METHYLCOBALAMIN\PYRIDOXAL 5-PHOSPHATE:Depression:0.47), (LEVOMEPROMAZINE HYDROCHLORIDE\LEVOMEPROMAZINE MALEATE:Depression:1.22), (LINCOMYCIN:Depression:0.06), (LINOLEIC ACID:Depression:0.42), (LIPASE:Depression:0.11), (LITHIUM OROTATE:Depression:0.88), (LOPINAVIR:Depression:0.01), (LYCOPENE:Depression:0.74), (LYNESTRENOL:Depression:0.57), (LYSINE HYDROCHLORIDE:Depression:0.64), (MAGNESIUM ACETATE:Depression:0.13), (MAGNESIUM ALUMINUM SILICATE:Depression:0.07), (MAGNESIUM STEARATE:Depression:0), (MALIC ACID:Depression:0.3), (MAXACALCITOL:Depression:1.33), (MECLIZINE\PYRIDOXINE: Depression:0.02), (MEFLOQUINE HYDROCHLORIDE:Depression:1.24), (MELDONIUM:Depression:0.24), (MENADIONE: Depression:0.04), (MENINGOCOCCAL GROUP B VACCINE:Depression:0.16), (MEPIVACAINE HYDROCHLORIDE:Depression:0.28), (MEPREDNISONE:Depression:1.15), (METFORMIN HYDROCHLORIDE\SAXAGLIPTIN:Depression:1.7), (METHAZOLAMIDE:Depression:0.42), (METHENAMINE MANDELATE:Depression:2.15), (METHENOLONE ENANTHATE:Depression:1.67), (METHSUXIMIDE:Depression:1.38), (METHYCLOTHIAZID E:Depression:0.18), (METHYL ALCOHOL:Depression:0.15), (METHYLPREDNISOLONE OR METHYLPREDNISOLONE ACETATE:Depression:0.3), (METHYSERGIDE MALEATE:Depression:0.75), (METRELEPTIN:Depression:2.57), (METYROSINE:Depression: 1.22), (MICONAZOLE NITRATE\PETROLATUM\ZINC OXIDE:Depression:0.17), (MIDOMAFETAMINE:Depression:0.42), (MIFAMURTIDE:Depression: 0.68), (MINERALS\PROBIOTICS NOS\VITAMINS:Depression:0.77), (MONOSODIUM GLUTAMATE:Depression:0.15), (NADH:Depression:1.27), (NALBUPHINE:Depression:0.3), (N ALFURAFINE HYDROCHLORIDE:Depression:0.27), (NALOXONE HYDROCHLORIDE\PENTAZOCINE HYDROCHLORIDE:Depression:0.8), (NAPHAZOLINE HYDROCHLORIDE:Depression:0.74), (NAPHAZOLINE NITRATE\PREDNISOLONE:Depression:0.79), (NEISSERIA MENINGITIDIS GROUP A CAPSULAR OLIGOSACCHARIDE DIPHTHERIA CRM197 PROTEIN CONJUGATE ANTIGEN\NEISSERIA MENINGITIDIS GROUP C CAPSULAR OLIGOSACCHARIDE DIPHTHERIA CRM197 PROTEIN CONJUGATE ANTIGEN\NEISSERIA MENINGITIDIS GROUP W-135 CAPSULAR OLIGOSACCHARIDE DIPHTHERIA CRM197 PROTEIN CONJUGATE ANTI:Depression:0.06), (NEISSERIA MENINGITIDIS GROUP A CAPSULAR POLYSACCHARIDE DIPHTHERIA TOXOID CONJUGATE ANTIGEN\NEISSERIA MENINGITIDIS GROUP C CAPSULAR POLYSACCHARIDE DIPHTHERIA TOXOID CONJUGATE ANTIGEN\NEISSERIA MENINGITIDIS GROUP W-135 CAPSULAR POLYSACCHARIDE DIPHTHERIA TOXOID CONJUGATE ANTIGEN\NEISSERIA MENINGITIDIS:Depression:0.19), (NELARABINE:Depression:0.5), (NEOMYCIN SULFATE:Depression:0.32), (NEOMYCIN\POLYMYXIN:Depression:0.13), (NEOSTIGMINE BROMIDE:Depression:0.6), (NEOSTIGMINE METHYLSULFATE:Depression:0.14), (NIACINAMIDE\PYRIDOXINE HYDROCHLORIDE\RIBOFLAVIN\THIAMINE HYDROCHLORIDE:Depression:0.03), (NIACINAMIDE\PYRIDOXINE\RIBOFLAVIN\THIA MINE:Depression:0.62), (NIAOULI OIL\QUININE BENZOATE\THIAMINE HYDROCHLORIDE:Depression:1.93), (NIFUROXAZIDE:Depression:0.22), (NIFURTOINOL: Depression:0.89), (NILUTAMIDE:Depression:0.78), (NITAZOXANIDE:Depression:0.64), (NIT ROGEN:Depression:0.12), (NOMEGESTROL:Depression:0.22), (NUSINERSEN:Depression:1.08), (OBETICHOLIC ACID:Depression:0.78), (OLODATEROL:Depression:0.46), (OLSALAZINE SODIUM:Depression:0.12), (OMACETAXINE:Depression:0.12), (OMBITASVIR HEMINONAHYDRATE\PARITAPREVIR DIHYDRATE\RITONAVIR:Depression:0.05), (OPICAPONE:Depression:1.78), (OREGANO: Depression:0.05), (ORNITHINE:Depression:0.14), (OSELTAMIVIR:Depression:2.76), (OSTREA EDULIS SHELL:Depression:0.77), (OXANDROLONE:Depression:0.78), (OXICONAZOLE NITRATE:Depression:0.04), (OXYMETAZOLINE HYDROCHLORIDE\PSEUDOEPHEDRINE HYDROCHLORIDE:Depression:0.06), (OXYMETHOLONE:Depression:0.01), (OXYPERTINE:Depression:0.68), (PALIVIZUMAB:Depression:0.14), (PAMIDRONIC ACID:Depression:0.2), (PANCRELIPASE\TYROSINE:Depression:0.26), (PARECOXIB SODIUM:Depression:0.06), (PARNAPARIN SODIUM:Depression:0.19), (PASCOPYRUM SMITHII POLLEN:Depression:0.5), (PASSIFLORA INCARNATA FLOWER:Depression:0.94), (PENBUTOLOL SULFATE:Depression:0.98), (PENTETRAZOL:Depression:0.07), (PENTOBARBITAL:Depression: 0.35), (PENTOBARBITAL SODIUM:Depression:0.36), (PENTOSTATIN:Depression:1.59), (PENTOXYVERINE:Depression: 0.77), (PEPPERMINT OIL:Depression:1.33), (PEPSIN:Depression:1.09), (PERAMIVIR:Depression:0.29), (PEROSPIR ONE:Depression:2.03), (PHENACETIN:Depression:0.05), (PHENCYCLIDINE:Depression:0.12), (PHENOXYBENZAMINE:Depression:0.24), (PHENOXYMETHYLPENICILLIN CALCIUM:Depression:0.35), (PHENYLEPHRINE HYDROCHLORIDE\PROMETHAZINE HYDROCHLORIDE:Depression:1.43), (PILSICAINIDE:Depression:0.12), (PIMOBENDAN: Depression:2.1), (PIPOTIAZINE:Depression:1.24), (PIRENZEPINE HYDROCHLORIDE:Depression:0.53), (PIRETANIDE:Depression:0.17), (PITOFENONE:Depression: 1.73), (PIZOTYLINE MALEATE:Depression:0.5), (PLANTAGO OVATA SEED COAT:Depression:0.7), (PLANTAGO OVATA SEED\PLANTAGO SEED:Depression:0.49), (PLASMA PROTEIN FRACTION (HUMAN):Depression:0.18), (POLIDOCANOL:Depression:0.26), (POLOXAMER:Depression:00.07), (POLYETHYLENE GLYCOL 400:Depression:1.15), (POLYSTYRENE SULFONIC ACID:Depression:0.55), (POTASSIUM BICARBONATE:Depression:0.66), (POTASSIUM BITARTRATE:Depression:0.33), (POTASSIUM BROMIDE:Depression:0.57), (POTASSIUM PHOSPHATE, DIBASIC\POTASSIUM PHOSPHATE, MONOBASIC:Depression:0.17), (POTASSIUM SORBATE:Depression:0.11), (PREDNISOLONE SODIUM METAZOATE:Depression:1.03), (PROCAINAMIDE HYDROCHLORIDE:Depression:0.36), (PROPOXYPHENE NAPSYLATE:Depression:0.83), (PSORALEN:Depression:0.74), (PYRIMETHAMINE\SULFA DOXINE:Depression:0.04), (QUINIDINE GLUCONATE:Depression:0.94), (QUININE BISULFATE:Depression:0.29), (RADIUM:Depression:0.68), (REISHI:Depression:0.55), (RESER PINE:Depression:0.38), (RESLIZUMAB:Depression:0.54), (RILMAZAFONE:Depression:0.97), (RUTIN:Depression:0.01), (RUXOLITINIB PHOSPHATE:Depression:0.14), (SAFINAMIDE:Depression:2.11), (SARPOGRELATE HYDROCHLORIDE:Depression:1.18), (SEBELIPASE ALFA:Depression:0.08), (SENNOSIDE A:Depression:0.21), (SENNOSIDE A\SENNOSIDE B\SENNOSIDES:Depression:0.34), (SHARK CARTILAGE\SHARK, UNSPECIFIED:Depression:0.74), (SHARK LIVER OIL:Depression:0.26), (SILTUXIMAB:Depression:0.19), (SITAFLOXACIN:Depression:0.22), (S ODIUM FLUORIDE\TRICLOSAN:Depression:0.21), (SODIUM FLUORIDE\VITAMINS:Depression:1.33), (SODIUM GUALENATE MONOHYDRATE:Depression:0.16), (SODIUM HYDROXIDE:Depression:0.02), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Depression:1.7), (SODIUM PHOSPHATE, MONOBASIC, ANHYDROUS:Depression:1.1), (SOLIFENACIN\TAMSULOSIN:Depression:0.67), (SOMATO STATIN:Depression:0.04), (SOMATREM:Depression:0.93), (SOY ISOFLAVONES:Depression:0.38), (STREPTOMYCIN\STREPTOMYCIN SULFATE:Depression:0.73), (STRONTIUM:Depression:0.54), (STRONTIUM CHLORIDE:Depression:0.04), (STRONTIUM RANELATE:Depression:0.51), (SULTHIAME:Depression:0.93), (SUPLATAST TOSILATE:Depression:1.56), (SUPROFEN:Depression:0.11), (TABALUMAB:Depression:0.18), (TAR:Depression:0.01), (TARAXACUM OFFICINALE POLLEN:Depression:0.27), (TAZOBACTAM SODIUM:Depression:0.14), (TBO-FILGRASTIM:Depression:0.39), (TECHNETIUM TC-99M SESTAMIBI\TETRAKIS(2-METHOXYISOBUTYLISOCYANIDE)COPPER(I) TETRAFLUOROBORATE:Depression:0.13), (TELITHROMYCIN:Depression:0.41), (TEMOC APRIL:Depression:0), (TENECTEPLASE:Depression:0.02), (TETRAHYDROZOLINE:Depression: 0.09), (THIABENDAZOLE:Depression:0.39), (THIAMYLAL SODIUM:Depression:0.19), (THIETHYLPERAZINE MALATE:Depression:0.21), (THIOCTIC ACID:Depression:2.03), (THROMBOMODULIN ALFA:Depression:0.06), (THYMOL:Depression:0.25), (THYROGLOBULIN:Depression:0.37), (TIAGABINE:Depression:0.2), (TIPEPIDINE HIBENZATE:Depression:0.42), (TIQUIZIUM BROMIDE:Depression:0.44), (TOBRAMYCIN SULFATE:Depression:0.15), (TOLMETIN:Depression:0.81), (TOLMETIN SODIUM:Depression:0.76), (TOLNAFTATE:Depression:0.3), (TOSUFLOXACIN TOSYLATE:Depression:0.24), (TRIENTINE HYDROCHLORIDE:Depression:0.54), (TRIMETHOPRIM SULFATE:Depression:0.52), (TRIPTORELIN PAMOATE:Depression:2.15), (TROMETHAMINE:Depression:0.43), (TROXIPIDE:Depression: 0.82), (ULIPRISTAL:Depression:0.85), (UNDECYLENIC ACID:Depression:0.72), (URACIL:Depression:0.32), (VALERIAN EXTRACT:Depression:0.46), (VALRUBICIN:Depression:0.64), (VITAMIN A PALMITATE:Depression:0.78), (VONOPRAZAN:Depression:0.41), (VORAPAXAR SULFATE:Depression:0.78), (WHEY:Depression:0.56), (XYLITOL:Depression:0.43), (XYLOM ETAZOLINE HYDROCHLORIDE:Depression:0.13), (YTTRIUM Y-90:Depression:0.14), (ZINC CITRATE:Depression:0.57), (ZIV-AFLIBERCEPT:Depression:1.28), (ZOTEPINE:Depression:1.78), (ALCOHOL:Depressive symptom:0.04), (BUPRENORPHINE HYDROCHLORIDE:Depressive symptom:0.1), (DALFAMPRIDINE:Depressive symptom:0.31), (DICLOFENAC:Depressive symptom:0.09), (ETHAMBUTOL HYDROCHLORIDE:Depressive symptom:0.87), (OMEPRAZOLE MAGNESIUM:Depressive symptom:0.15), (SPIRONOLACTONE:Depressive symptom:0), (TERIFLUNOMIDE:Depressive symptom:0), (TOPIRAMATE:Depressive symptom:0.33), (ABIRATERONE ACETATE:Dermatitis:0.01), (ACETAMINOPHEN\BUTALBITAL\CAFFEINE:Dermatitis:0.75), (ALBUTEROL SULFATE\IPRATROPIUM BROMIDE:Dermatitis:0.54), (BENZONATATE:Dermatitis:0.24), (BORTEZOMIB:Dermatitis:0.94), (CALCITRIOL:Dermatitis:0.06), (CALCIUM CITRATE:Dermatitis:0.21), (CANDESARTAN CILEXETIL\HYDROCHLOROTHIAZIDE:Dermatitis:0.59), (CARFILZOMIB:Dermatitis:0.45), (CARISOPRODOL:Dermatitis:0.2), (CARVEDILOL PHOSPHATE:Dermatitis:0.25), (CETIRIZINE HYDROCHLORIDE\PSEUDOEPHEDRINE HYDROCHLORIDE:Dermatitis:0), (CHOLESTYRAMINE:Dermatitis:0.99), (CODEINE:Dermatitis: 0.29), (DASATINIB:Dermatitis:1.22), (DEVICE:Dermatitis:0.08), (DIETARY SUPPLEMENT:Dermatitis:0.27), (DIMENHYDRINATE:Dermatitis:0.16), (DIVALPROEX SODIUM:Dermatitis:0.71), (DOCUSATE CALCIUM:Dermatitis:0.06), (DULOXETINE:Dermatitis:0.67), (EDOXABAN TOSYLATE:Dermatitis:0.36), (ELETRIPTAN HYDROBROMIDE:Dermatitis:0.56), (ERGOCALCIFEROL:Dermatitis:0.79), (ERYTHROPOIE TIN:Dermatitis:0.33), (ESOMEPRAZOLE:Dermatitis:0.9), (ESOMEPRAZOLE MAGNESIUM\NAPROXEN:Dermatitis:0.12), (ESTROGENS, CONJUGATED\MEDROXYPROGESTERONE ACETATE:Dermatitis:0.1), (ETHAMBUTOL HYDROCHLORIDE:Dermatitis:0.93), (EZETIMIBE\SIMVASTATIN:Dermatitis:0.26), (FENT ANYL CITRATE:Dermatitis:0.14), (FESOTERODINE FUMARATE:Dermatitis:0.37), (FLECAINIDE:Dermatitis:0.14), (FULVESTRANT:Dermatitis:0.36), (HERBALS\TURMERIC:Dermatitis:0.02), (HYDROCHLOROTHIAZIDE\LOSARTAN POTASSIUM:Dermatitis:0.26), (HYDROCORTISONE SODIUM SUCCINATE:Dermatitis:0.17), (HYDROXYCHLOROQUINE DIPHOSPHATE:Dermatitis:0.4), (HYDROXYUREA:Dermatitis:0), (HYDROXYZINE PAMOATE:Dermatitis:0.64), (HYOSCYAMINE:Dermatitis:0.04), (IMATINIB MESYLATE:Dermatitis:0.42), (IMIPRAMINE HYDROCHLORIDE:Dermatitis:0.11), (INFLIXIMAB-DYYB:Dermatitis:0.04), (IRINOTECAN HYDROCHLORIDE:Dermatitis:0.4), (ISOSORBIDE:Dermatitis:0.35), (LABETALOL\LABET ALOL HYDROCHLORIDE:Dermatitis:0.08), (LATANOPROST:Dermatitis:0.49), (LERCANIDIPINE: Dermatitis:0.4), (LERCANIDIPINE HYDROCHLORIDE:Dermatitis:0.15), (LIDOCAINE\PRILOCAINE:Dermatitis:0.25), (LITHIU M CARBONATE:Dermatitis:0.58), (MEGESTROL ACETATE:Dermatitis:0.18), (METAXALONE:Dermatitis:0.58), (METHOCARBAMOL:Dermatitis: 0.28), (NEBIVOLOL HYDROCHLORIDE:Dermatitis:0.31), (NIACIN:Dermatitis:0.42), (OXCARBAZEPINE:Dermatitis: 1.05), (PAMIDRONATE DISODIUM:Dermatitis:0.08), (PANTOPRAZOLE MAGNESIUM:Dermatitis:0.53), (PAROXETINE\PAROXETINE HYDROCHLORIDE:Dermatitis:0.46), (PENICILLIN:Dermatitis:0.24), (PERINDOPRIL:Dermatitis: 0.17), (POTASSIUM:Dermatitis:1.12), (PRAMIPEXOLE:Dermatitis:0.29), (PRAMIPEXOLE DIHYDROCHLORIDE:Dermatitis:0.42), (PREDNISOLONE\PREDNISOLONE ACETATE\PREDNISOLONE SODIUM SUCCINATE:Dermatitis:0.56), (PROMETHAZINE\PROMETHAZINE HYDROCHLORIDE:Dermatitis:0.59), (PROPRANOLOL\PROPRANOLOL HYDROCHLORIDE:Dermatitis:0.15), (QUETIAPINE:Dermatitis:0.5), (RANOLAZINE:Dermatitis: 0.46), (REGORAFENIB:Dermatitis:0.39), (RISEDRONATE SODIUM:Dermatitis:0.17), (ROFLUMILAST:Dermatitis:0.23), (ROPINIROLE HYDROCHLORIDE:Dermatitis:0.04), (SCOPOLAMINE:Dermatitis:0.32), (SUMATRIPTAN\S UMATRIPTAN SUCCINATE:Dermatitis:0.3), (TERBUTALINE SULFATE:Dermatitis:0.2), (TERIFLUNOMIDE:Dermatitis:0.43), (VANCOMYCIN HYDROCHLORIDE:Dermatitis:0.01), (VARENICLINE TARTRATE:Dermatitis:0.96), (ACETAMINOPHEN\CODEINE PHOSPHATE:Dermatitis atopic:0.12), (BUMETANIDE:Dermatitis atopic:0.19), (BUSPIRONE HYDROCHLORIDE:Dermatitis atopic:0.05), (CERTOLIZUMAB PEGOL:Dermatitis atopic:0.69), (DICLOFENAC:Dermatitis atopic:0.54), (DILTIAZEM:Dermatitis atopic:0.09), (DILTIAZEM HYDROCHLORIDE:Dermatitis atopic:0.3), (DIVALPROEX SODIUM:Dermatitis atopic:0.98), (DULOXETINE:Dermatitis atopic:0), (ENOXAPARIN:Dermatitis atopic:0.12), (EVOLOCUMAB:Dermatitis atopic:0.63), (FERROUS SULFATE\FERROUS SULFATE, DRIED:Dermatitis atopic:0.29), (IBRUTINIB:Dermatitis atopic:0.52), (LEDIPASVIR\SOFOSBUVIR:Dermatitis atopic:0.01), (LENALIDOMIDE:Dermatitis atopic:0.22), (METRONIDAZOLE:Dermatitis atopic:0.84), (MOXIFLOXACIN HYDROCHLORIDE:Dermatitis atopic:0.06), (PRAVASTATIN:Dermatitis atopic:0.08), (SACUBITRIL\VALSARTAN:Dermatitis atopic:0.02), (SODIUM OXYBATE:Dermatitis atopic:0.21), (TERIFLUNOMIDE:Dermatitis atopic:0.37), (UMECLIDINIUM BROMIDE\VILANTEROL TRIFENATATE:Dermatitis atopic:0.09), (VARENICLINE TARTRATE:Dermatitis atopic:0.57), (ZOLPIDEM\ZOLPIDEM TARTRATE:Dermatitis atopic:0.1), (PYRAZINAMIDE:Dermatitis bullous:0.58), (CYCLOBENZAPRINE:Dermatitis contact:0.1), (DALFAMPRIDINE:Dermatitis contact:0.06), (DIVALPROEX SODIUM:Dermatitis contact:0.43), (EVOLOCUMAB:Dermatitis contact:0.3), (FERROUS SULFATE:Dermatitis contact:0.26), (LOVASTATIN:Dermatitis contact:0.04), (OMEPRAZOLE MAGNESIUM:Dermatitis contact:0.4), (OXCARBAZEPINE:Dermatitis contact:0.55), (PANTOPRAZOLE:Dermatitis contact:0.28), (PRAVASTATIN SODIUM:Dermatitis contact:0.13), (TERIFLUNOMIDE:Dermatitis contact:0.16), (TIOTROPIUM BROMIDE MONOHYDRATE:Dermatitis contact:0.08), (TIZANIDINE HYDROCHLORIDE:Dermatitis contact:0.12), (AMIODARONE:Dermatomyositis:0.12), (BUMETANIDE:Dermatomyositis:0.52), (CYCLOBENZAPRINE:Dermatomyositis:0.14), (DEVICE:Dermatomyositis:0.04), (DEXLANS OPRAZOLE:Dermatomyositis:0.5), (DICLOFENAC:Dermatomyositis:0.65), (ESZOPICLONE: Dermatomyositis:0.21), (ETHAMBUTOL HYDROCHLORIDE:Dermatomyositis:0.92), (ETORICOXIB:Dermatomyositis:0.24), (EVEROL IMUS:Dermatomyositis:0.34), (EVOLOCUMAB:Dermatomyositis:0.69), (GLIPIZIDE:Dermatomyositis: 0.21), (IBRUTINIB:Dermatomyositis:0.76), (LEDIPASVIR\SOFOSBUVIR:Dermatomyositis: 0), (LORATADINE:Dermatomyositis:0.09), (LOVASTATIN:Dermatomyositis:0.38), (MA GNESIUM:Dermatomyositis:0.34), (MONTELUKAST SODIUM:Dermatomyositis:0.24), (NEBIVOLOL:Dermatomyositis:0.26), (PRAVASTATIN: Dermatomyositis:0.41), (SECUKINUMAB:Dermatomyositis:0.68), (SELEXIPAG:Dermatomyositis: 0.12), (TOPIRAMATE:Dermatomyositis:0.67), (VERAPAMIL HYDROCHLORIDE:Dermatomyositis:0.45), (ALBUMIN HUMAN:Device related infection:0.24), (ALENDRONIC ACID:Device related infection:0.2), (ANASTROZOLE:Device related infection:0.18), (BISACODYL OR DOCUSATE SODIUM:Device related infection:0.16), (BORTEZOMIB:Device related infection:0.04), (CALCITRIOL:Device related infection:0.22), (CANDESARTAN:Device related infection:0.13), (CANDESARTAN CILEXETIL\HYDROCHLOROTHIAZIDE:Device related infection:0.39), (CARFILZOMIB:Device related infection:0.06), (CASPOFUNGIN:Device related infection:0.51), (CERTOLIZUMAB PEGOL:Device related infection:0.72), (CETUXIMAB:Device related infection:0.3), (CLOTRIMAZOLE:Device related infection:0.15), (CLOZAPINE:Device related infection:0.33), (COLCHICINE:Device related infection:0.38), (CORTISONE\HYDROCORTISONE:Device related infection:0.26), (DASATINIB:Device related infection:0.39), (DEXLANSOPRAZOLE:Device related infection:0.27), (DIVALPROEX SODIUM:Device related infection:0.45), (ECONAZOLE:Device related infection:0.23), (EPIRUBICIN:Device related infection:0.12), (ERYTHROPOIETIN:Device related infection:0.34), (ESZOPICLONE:Device related infection:0.17), (EVOLOCUMAB:Device related infection:0.03), (FENOFIBRATE:Device related infection:0.02), (HYDROMORPHONE HYDROCHLORIDE:Device related infection:0.21), (IBRUTINIB:Device related infection:0.93), (ILOPROST:Device related infection:0.02), (INVESTIGATIONAL PRODUCT:Device related infection:0.03), (IRINOTECAN HYDROCHLORIDE:Device related infection:0.41), (IRON:Device related infection:0.39), (LEDIPASVIR\SOFOSBUVIR:Device related infection:0.09), (LITHIUM CARBONATE:Device related infection:0.21), (LOVASTATIN:Device related infection:0.38), (MELOXICAM:Device related infection:0.17), (METAMIZOLE:Device related infection:0.08), (METHYLPREDNISOLONE SODIUM SUCCINATE:Device related infection:1.24), (NILOTINIB:Device related infection:0.95), (NOREPINEPHRINE:Device related infection:0), (OSELTAMIVIR:Device related infection:0.76), (OXCARBAZEPINE:Device related infection:0.67), (PANTOPRAZOLE MAGNESIUM:Device related infection:0.05), (PONATINIB:Device related infection:0.07), (QUETIAPINE:Device related infection:0.33), (SECUKINUMAB:Device related infection:0.01), (SOTALOL:Device related infection:0.26), (SULFAMETHOXAZOLE:Device related infection:0.09), (TELMISARTAN:Device related infection:0.38), (THALIDOMIDE:Device related infection:0.32), (TINZAPARIN SODIUM:Device related infection:0.18), (TIOTROPIUM:Device related infection:0.08), (VALGANCICLOVIR HYDROCHLORIDE:Device related infection:0.51), (DIVALPROEX SODIUM:Diabetes insipidus:0.04), (HYDROXYCHLOROQUINE SULFATE:Diabetes insipidus:0.13), (IBRUTINIB:Diabetes insipidus:0.15), (OXCARBAZEPINE:Diabetes insipidus:0.27), (.ALPHA.-TOCOPHEROL ACETATE, D-\ASCORBIC ACID\CALCIUM PANTOTHENATE\FOLIC ACID\MANGANESE\NIACINAMIDE\PYRIDOXINE\RIBOFLAVIN\THIAMINE\VITAMIN A\ZINC OXIDE:Diabetes mellitus:0.11), (.ALPHA.-TOCOPHEROL SUCCINATE, D-\ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\SELENOCYSTEINE\THIAMINE MONONITRATE\ZINC CITRATE:Diabetes mellitus:0.59), (.GAMMA.-AMINOBUTYRIC ACID:Diabetes mellitus:0.13), (ABALOPARATIDE:Diabetes mellitus:0.04), (ACAMPROSATE:Diabetes mellitus:1.23), (ACETAMINOPHEN AND OR DEXTROMETHORPHAN AND OR DIPHENHYDRAMINE AND OR GUAIFENESIN AND OR PHENYLEPHRINE AND OR PSEUDOEPHEDRINE:Diabetes mellitus:0.14), (ACETAMINOPHEN\ASPIRIN:Diabetes mellitus:0.09), (ACETAMINOPHEN\ASPIRIN\SALICYLAMIDE:Diabetes mellitus:0.23), (ACETAMINOPHEN\BELLADONNA LEAF\CAFFEINE\MORPHINE:Diabetes mellitus:0.25), (ACETAMINOPHEN\BELLADONNA LEAF\CAFFEINE\OPIUM TINCTURE:Diabetes mellitus:0.31), (ACETAMINOPHEN\BUTALBITAL\CAFFEINE\CODEINE PHOSPHATE:Diabetes mellitus:1.35), (ACETAMINOPHEN\BUTYLSCOPOLAMINE BROMIDE:Diabetes mellitus:0.94), (ACETAMINOPHEN\CAFFEINE\CODEINE PHOSPHATE:Diabetes mellitus:0.34), (ACETAMINOPHEN\CHLORPHENIRAMINE MALEATE\PHENYLEPHRINE HYDROCHLORIDE OR CETIRIZINE HYDROCHLORIDE OR CHLORPHENIRAMINE MALEATE OR CLEMASTINE FUMARATE OR DIPHENHYDRAMINE HYDROCHLORIDE OR FEXOFENADINE HYDROCHLORIDE OR LORATADINE:Diabetes mellitus:0.42), (ACETAMINOPHEN\DEXTROMETHORPHAN HYDROBROMIDE\PHENYLEPHRINE HYDROCHLORIDE:Diabetes mellitus:0.19), (ACETAMINOPHEN\DICHLORALPHENAZONE\ISOMETHEPTENE HYDROCHLORIDE:Diabetes mellitus:0.27), (ACETAMINOPHEN\DIPHENHYDRAMINE\DIPHENHYDRAMINE CITRATE:Diabetes mellitus:0.06), (ACETAMINOPHEN\PHENYLEPHRINE HYDROCHLORIDE:Diabetes mellitus:0.42), (ACETAMINOPHEN\PSEUDOEPHEDRINE HYDROCHLORIDE:Diabetes mellitus:0.75), (ACETOHYDROXAMIC ACID:Diabetes mellitus:0.52), (ACETYLCYSTEINE\BENZALKONIUM CHLORIDE\TUAMINOHEPTANE SULFATE:Diabetes mellitus:0.83), (ACLARUBICIN HYDROCHLORIDE:Diabetes mellitus:0.28), (ADAPALENE:Diabetes mellitus:0.35), (ADENINE:Diabetes mellitus:1.95), (ADENOSINE TRIPHOSPHATE DISODIUM:Diabetes mellitus:0.82), (ADUCANUMAB:Diabetes mellitus:1.06), (ALBUTEROL\GUAIFENESIN:Diabetes mellitus:1.17), (ALCOHOL\ALUMINUM CHLORIDE:Diabetes mellitus:0.38), (ALDESLEUKIN:Diabetes mellitus:0.56), (ALFALFA:Diabetes mellitus:0.96), (ALISPORIVIR:Diabetes mellitus:0.55), (ALOSETRON:Diabetes mellitus:0.07), (ALPHA-TOCOPHEROL\ASCORBIC ACID\CHOLECALCIFEROL\COPPER\CYANOCOBALAMIN\FOLIC ACID\LUTEIN\OMEGA-3 FATTY ACIDS\PYRIDOXINE HYDROCHLORIDE\TAURINE\VITAMIN A PALMITATE\ZEAXANTHIN\ZINC PICOLINATE:Diabetes mellitus:0.17), (ALUMINUM CHLOROHYDRATE:Diabetes mellitus:0.43), (ALUMINUM HYDROXIDE\DIMETHICONE\DIPHENHYDRAMINE\HYDROXIDE ION\LIDOCAINE:Diabetes mellitus:0.45), (ALUMINUM HYDROXIDE\MAGNESIUM HYDROXIDE\MAGNESIUM TRISILICATE:Diabetes mellitus:0.26), (ALUMINUM HYDROXIDE\MAGNESIUM TRISILICATE:Diabetes mellitus:0.2), (ALVERINE CITRATE:Diabetes mellitus:0.16), (AMINO ACIDS\VITAMINS:Diabetes mellitus:1.06), (AMLODIPINE\INDAPAMIDE:Diabetes mellitus:0.1), (AMMONIUM CHLORIDE\DEXTROMETHORPHAN\SODIUM CITRATE:Diabetes mellitus:1.84), (AMOBARBITAL SODIUM:Diabetes mellitus:0.47), (AMOBARBITAL\BELLADONNA LEAF\DIMETHICONE\ERGOTAMINE TARTRATE:Diabetes mellitus:0.32), (AMOXICILLIN SODIUM:Diabetes mellitus:0.63), (AMPHETAMINE SULFATE:Diabetes mellitus:0.13), (AMPHETAMINE\DEXTROAMPHETAMINE:Diabetes mellitus:0.26), (AMPICILLIN SODIUM:Diabetes mellitus:2.28), (AMYLASE:Diabetes mellitus:0.55), (AMYLMETACRESOL\DICHLOROBENZYL ALCOHOL:Diabetes mellitus:0.04), (APOMORPHINE:Diabetes mellitus:0.14), (ARNICA MONTANA:Diabetes mellitus:0.07), (ASCORBIC ACID\CALCIUM PANTOTHENATE\CYANOCOBALAMIN\NIACINAMIDE\PYRIDOXINE HYDROCHLORIDE\RIBOFLAVIN\THIAMINE MONONITRATE:Diabetes mellitus:0.72), (ASCORBIC ACID\CHONDROITIN SULFATE (BOVINE)\GLUCOSAMINE SULFATE\MANGANESE:Diabetes mellitus:0.51), (ASCORBIC ACID\FERROUS ASPARTO GLYCINATE\FERROUS FUMARATE\FOLIC ACID\NIACIN:Diabetes mellitus:0.07), (ASCORBIC ACID\MINERALS\VITAMINS:Diabetes mellitus:0.65), (ASPARTIC ACID:Diabetes mellitus:0.47), (ASPIRIN\CAFFEINE\ORPHENADRINE CITRATE:Diabetes mellitus:0.22), (ASPIRIN\CAFFEINE\SALICYLAMIDE:Diabetes mellitus:0.91), (ASPIRIN\CARISOPRODOL:Diabetes mellitus:0.27), (ASPIRIN\CHLORPHENIRAMINE MALEATE\PHENYLEPHRINE BITARTRATE:Diabetes mellitus:0.07), (ASPIRIN\DIHYDROXYALUMINUM AMINOACETATE\MAGNESIUM CARBONATE:Diabetes mellitus:0.98), (ATAZANAVIR\RITONAVIR:Diabetes mellitus:0.61), (ATENOLOL\NIFEDIPINE:Diabetes mellitus:0.42), (ATOMOXETINE:Diabetes mellitus:0.5), (ATRACURIUM BESYLATE:Diabetes mellitus:0.68), (ATROPINE SULFATE\DIFENOXIN HYDROCHLORIDE:Diabetes mellitus:0.76), (AVOBENZONE\HOMOSALATE\OCTISALATE:Diabetes mellitus:1.39), (AZITHROMYCIN OR METHOTREXATE SODIUM:Diabetes mellitus:2.49), (BACILLUS CALMETTE-GUERIN SUBSTRAIN CONNAUGHT LIVE ANTIGEN:Diabetes mellitus:0.12), (BACITRACIN\NEOMYCIN SULFATE:Diabetes mellitus:0.99), (BACITRACIN\NEOMYCIN SULFATE\POLYMYXIN B SULFATE:Diabetes mellitus:0.62), (BALSAM PERU OIL\CASTOR OIL\TRYPSIN:Diabetes mellitus:0.97), (BALSAM PERU\CASTOR OIL\TRYPSIN:Diabetes mellitus:0.07), (BELLADONNA\ERGOTAMINE TARTRATE\PHENOBARBITAL:Diabetes mellitus:0.04), (BENZETHONIUM CHLORIDE:Diabetes mellitus:0.44), (BENZOCAINE\RESORCINOL:Diabetes mellitus:0.25), (BENZOIC ACID\HYOSCYAMINE SULFATE\METHENAMINE\METHYLENE BLUE\PHENYL SALICYLATE:Diabetes mellitus:0.34), (BEPOTASTINE\BEPOTASTINE BESILATE:Diabetes mellitus:0.3), (BETA GLUCAN:Diabetes mellitus:0.8), (BETAMETHASONE ACETATE\BETAMETHASONE SODIUM PHOSPHATE:Diabetes mellitus:0.15), (BETAMETHASONE SODIUM PHOSPHATE\NEOMYCIN SULFATE:Diabetes mellitus:1.3), (BIFIDOBACTERIUM BIFIDUM:Diabetes mellitus:0.22), (BIOTIN\CALCIUM PANTOTHENATE\CHOLINE BITARTRATE\CYANOCOBALAMIN\FOLIC ACID\INOSITOL\NIACINAMIDE\PYRIDOXINE\RIBOFLAVIN\THIAMINE:Diabetes mellitus:0.37), (BIPERIDEN LACTATE:Diabetes mellitus:0.05), (BISACODYL\GLYCERIN\MINERAL OIL\SODIUM PHOSPHATE, DIBASIC\SODIUM PHOSPHATE, MONOBASIC:Diabetes mellitus:1.27), (BISMUTH SUBSALICYLATE\GLYCERIN\PROPYLENE GLYCOL:Diabetes mellitus:0.46), (BLESELUMAB:Diabetes mellitus:0.23), (BORON:Diabetes mellitus:0.84), (BOTULINUM TOXIN TYPE A:Diabetes mellitus:1.97), (BOTULISM ANTITOXIN:Diabetes mellitus:0.24), (BRIMONIDINE TARTRATE\DORZOLAMIDE HYDROCHLORIDE\TIMOLOL MALEATE:Diabetes mellitus:0.45), (BRODALUMAB:Diabetes mellitus:0.19), (BROMFENAC\BROMFENAC SODIUM:Diabetes mellitus:0.02), (BROMPHENIRAMINE MALEATE\PSEUDOEPHEDRINE HYDROCHLORIDE:Diabetes mellitus:0.16), (BUFLOMEDIL:Diabetes mellitus:0.07), (BUPROPION HYDROBROMIDE:Diabetes mellitus:0.83), (BUTYLSCOPOLAMINE BROMIDE\METAMIZOLE SODIUM:Diabetes mellitus:0.66), (CAFFEINE\PHENOBARBITAL:Diabetes mellitus:0.06), (CAJUPUT OIL\EUCALYPTUS OIL\PEPPERMINT OIL:Diabetes mellitus:1.33), (CALCIUM ACETATE\MAGNESIUM ACETATE\POTASSIUM ACETATE\SODIUM ACETATE\SODIUM CHLORIDE:Diabetes mellitus:1.05), (CALCIUM ASCORBATE:Diabetes mellitus:0.7), (CALCIUM BICARBONATE:Diabetes mellitus:1.95), (CALCIUM CARBIMIDE:Diabetes mellitus:0.35), (CALCIUM CARBONATE\DIMETHICONE:Diabetes mellitus:0.25), (CALCIUM CHLORIDE\DEXTROSE MONOHYDRATE\HYDROCHLORIC ACID\MAGNESIUM CHLORIDE\SODIUM BICARBONATE\SODIUM CHLORIDE\SODIUM HYDROXIDE\SODIUM LACTATE:Diabetes mellitus:0.64), (CALCIUM CHLORIDE\MAGNESIUM CHLORIDE\POTASSIUM CHLORIDE\POTASSIUM PHOSPHATE, DIBASIC\POTASSIUM PHOSPHATE, MONOBASIC\SODIUM CHLORIDE:Diabetes mellitus:0.15), (CALCIUM CHLORIDE\MAGNESIUM CHLORIDE\POTASSIUM CHLORIDE\SODIUM ACETATE\SODIUM CHLORIDE:Diabetes mellitus:0.26), (CALCIUM CHLORIDE\POTASSIUM CHLORIDE\SODIUM CHLORIDE:Diabetes mellitus:0.2), (CALCIUM CL\DEXTROSE\MAGNESIUM CL\SODIUM BICAR\SODIUM CL\SODIUM LACT:Diabetes mellitus:0.38), (CALCIUM GLYCEROPHOSPHATE:Diabetes mellitus:0.85), (CALCIUM LACTATE\CALCIUM PHOSPHATE\CHOLECALCIFEROL:Diabetes mellitus:0.06), (CALCIUM PANTOTHENATE\CUPRIC SULFATE\CYANOCOBALAMIN\FERROUS ASPARTO GLYCINATE\FERROUS FUMARATE\FOLIC ACID\MAGNESIUM SULFATE\NIACINAMIDE\PYRIDOXINE HYDROCHLORIDE\RIBOFLAVIN\SODIUM ASCORBATE\THIAMINE MONONITRATE\ZINC SULFATE:Diabetes mellitus:0.23), (CALCIUM PANTOTHENATE\NIACINAMIDE\PYRIDOXINE HYDROCHLORIDE\RIBOFLAVIN\THIAMINE HYDROCHLORIDE:Diabetes mellitus:0.39), (CALCIUM PANTOTHENATE\NIACINAMIDE\PYRIDOXINE HYDROCHLORIDE\RIBOFLAVIN\THIAMINE MONONITRATE:Diabetes mellitus:0.08), (CALCIUM PHOSPHATE, DIBASIC, ANHYDROUS\FERROUS SUCCINATE\MAGNESIUM HYDROXIDE:Diabetes mellitus:0.23), (CALCIUM SULFATE:Diabetes mellitus:0.13), (CAMPHOR (SYNTHETIC)\CAPSICUM OLEORESIN\MENTHOL\METHYL SALICYLATE:Diabetes mellitus:0.01), (CAMPHOR (SYNTHETIC)\EUCALYPTUS OIL\MENTHOL:Diabetes mellitus:0.06), (CAMPHOR\DIPHENHYDRAMINE\ZINC OXIDE:Diabetes mellitus:1.32), (CANNABIS SATIVA SUBSP. SATIVA FLOWERING TOP:Diabetes mellitus:0.21), (CAPECITABINE\OXALIPLATIN:Diabetes mellitus:0.12), (CARBAMIDE PEROXIDE:Diabetes mellitus:1.24), (CARBOMER 934\GLYCERIN\METHYLPARABEN\PROPYLPARABEN\SODIUM HYDROXIDE:Diabetes mellitus:0.24), (CARBON DIOXIDE:Diabetes mellitus:0.08), (CARBOPLATIN\PACLITAXEL:Diabetes mellitus:0.16), (CARBOXYMETHYLCELLULOSE SODIUM\GELATIN\PECTIN:Diabetes mellitus:0.47), (CARBOXYMETHYLCELLULOSE SODIUM\HYPROMELLOSES:Diabetes mellitus:0.21), (CARBOXYMETHYLCELLULOSE SODIUM\SORBINICATE:Diabetes mellitus:0.83), (CARPRONIUM CHLORIDE:Diabetes mellitus:1.04), (CARRAGEENAN\TITANIUM\ZINC:Diabetes mellitus:0.02), (CAT'S CLAW:Diabetes mellitus:0.17), (CEFOPERAZONE:Diabetes mellitus:0.22), (CEFOTETAN DISODIUM:Diabetes mellitus:0.02), (CEFOXITIN\CEFOXITIN SODIUM:Diabetes mellitus:0.46), (CEFPIROME SULFATE:Diabetes mellitus:0.26), (CEFTERAM PIVOXIL:Diabetes mellitus:0.86), (CERTOLIZUMAB:Diabetes mellitus:0.55), (CETRAXATE HYDROCHLORIDE:Diabetes mellitus:0.32), (CHAMOMILE:Diabetes mellitus:0.16), (CHLORAMPHENICOL\PREDNISOLONE:Diabetes mellitus:0.19), (CHLORHEXIDINE HYDROCHLORIDE\NEOMYCIN SULFATE:Diabetes mellitus:0.36), (CHLORIDE ION:Diabetes mellitus:0.51), (CHLORMEZANONE:Diabetes mellitus:0.32), (CHLORPROMAZINE\PROMETHAZINE:Diabetes mellitus:0.54), (CHOLECALCIFEROL\GENISTEIN\ZINC GLYCINATE:Diabetes mellitus:0.87), (CHOLIC ACID:Diabetes mellitus:0.52), (CHOLINE:Diabetes mellitus:0.14), (CHOLINE BITARTRATE:Diabetes mellitus:0.07), (CHOLINE\TRAZODONE HYDROCHLORIDE:Diabetes mellitus:0.88), (CILASTATIN:Diabetes mellitus:0.15), (CIMETIDINE\FAMOTIDINE\RANITIDINE HYDROCHLORIDE:Diabetes mellitus:0.1), (CIPROFLOXACIN\CIPROFLOXACIN HYDROCHLORIDE:Diabetes mellitus:0.65), (CIPROFLOXACIN\DEXAMETHASONE:Diabetes mellitus:1.4), (CITRIC ACID MONOHYDRATE\DEXTROSE\SODIUM CITRATE:Diabetes mellitus:0.01), (CITRIC ACID MONOHYDRATE\TRISODIUM CITRATE DIHYDRATE:Diabetes mellitus:0.79), (CLEBOPRIDE MALATE\DIMETHICONE:Diabetes mellitus:0.25), (CLIDINIUM BROMIDE:Diabetes mellitus:0.02), (CLOBETASONE BUTYRATE\NYSTATIN\OXYTETRACYCLINE CALCIUM:Diabetes mellitus:0.13), (CLOCORTOLONE PIVALATE:Diabetes mellitus:1.13), (CLOMIPHENE CITRATE:Diabetes mellitus:0.09), (CLONIXIN\CYCLOBENZAPRINE:Diabetes mellitus:0.46), (CLOPREDNOL:Diabetes mellitus:0.51), (CLOSTRIDIUM TETANI TOXOID ANTIGEN (FORMALDEHYDE INACTIVATED)\CORYNEBACTERIUM DIPHTHERIAE TOXOID ANTIGEN (FORMALDEHYDE INACTIVATED):Diabetes mellitus:0.25), (CLOSTRIDIUM TETANI\CLOSTRIDIUM TETANI TOXOID ANTIGEN (FORMALDEHYDE INACTIVATED)\CORYNEBACTERIUM DIPHTHERIAE\CORYNEBACTERIUM DIPHTHERIAE TOXOID ANTIGEN (FORMALDEHYDE INACTIVATED):Diabetes mellitus:1.34), (COAGULATION FACTOR IX HUMAN\COAGULATION FACTOR VII HUMAN\COAGULATION FACTOR X HUMAN\PROTEIN C\PROTEIN S HUMAN\PROTHROMBIN:Diabetes mellitus:0.51), (COLCHICINE\OPIUM\TIEMONIUM METHYLSULFATE:Diabetes mellitus:0.47), (COPPER GLUCONATE:Diabetes mellitus:0.2), (COPPER OR LEVONORGESTREL:Diabetes mellitus:0.13), (CRATAEGUS LAEVIGATA FRUIT:Diabetes mellitus:0.18), (CREATININE:Diabetes mellitus:0.5), (CROFELEMER:Diabetes mellitus:0.84), (CYANAMIDE:Diabetes mellitus:0.81), (CYANOCOBALAMIN\PYRIDOXINE HYDROCHLORIDE\THIAMINE DISULFIDE:Diabetes mellitus:1.39), (CYCLOPHOSPHAMIDE\CYTARABINE\DEXAMETHASONE\DOXORUBIC IN\METHOTREXATE\VINCRISTINE:Diabetes mellitus:0.19), (CYPROTERONE ACETATE\ETHINYL ESTRADIOL:Diabetes mellitus:1.59), (CYSTEINE:Diabetes mellitus:0.86), (CYSTEINE\GLYCINE\GLYCYRRHIZIN:Diabetes mellitus:0.16), (CYTARABINE OCFOSFATE:Diabetes mellitus:0.18), (DACLIZUMAB:Diabetes mellitus:2.74), (DARUNAVIR\RITONAVIR:Diabetes mellitus:0.21), (DEMECLOCYCLINE:Diabetes mellitus:0.96), (DEMECLOCYCLINE HYDROCHLORIDE:Diabetes mellitus:0.5), (DEOXYRIBONUCLIEC ACID:Diabetes mellitus:0.2), (DEUTETRABENAZINE:Diabetes mellitus:0.21), (DEXIBUPROFEN:Diabetes mellitus:0.99), (DEXTRAN SULFATE SODIUM SALT:Diabetes mellitus:0.94), (DEXTROMETHORPHAN HYDROBROMIDE\GUAIFENESIN\PSEUDOEPHEDRINE HYDROCHLORIDE:Diabetes mellitus:0.37), (DEXTROSE MONOHYDRATE\SODIUM CHLORIDE:Diabetes mellitus:0.72), (DEXTROSE\SODIUM BICARBONATE\SODIUM CHLORIDE\SODIUM LACTATE:Diabetes mellitus:0.62), (DIBUCAINE:Diabetes mellitus:0.38), (DICLOFENAC DIETHYLAMINE:Diabetes mellitus:1.04), (DICLOXACILLIN SODIUM:Diabetes mellitus:0.56), (DIMETHYL SULFONE\GLUCOSAMINE SULFATE:Diabetes mellitus:0.35), (DIMETHYL SULFOXIDE:Diabetes mellitus:1.39), (DIPHENHYDRAMINE HYDROCHLORIDE\PHENYLEPHRINE HYDROCHLORIDE:Diabetes mellitus:0.46), (DOCUSATE SODIUM\SENNOSIDE A\SENNOSIDE B:Diabetes mellitus:0.27), (DOLASETRON:Diabetes mellitus:1.13), (DOLASETRON MESYLATE:Diabetes mellitus:2.36), (DOXIFLURIDINE:Diabetes mellitus:0.19), (DOXYCYCLINE HYDROCHLORIDE:Diabetes mellitus:1.48), (DOXYLAMINE SUCCINATE\PYRIDOXINE HYDROCHLORIDE:Diabetes mellitus:0.17), (DYCLONINE HYDROCHLORIDE:Diabetes mellitus:0.09), (DYDROGESTERONE\ESTRADIOL:Diabetes mellitus:1.19), (EFALIZUMAB:Diabetes mellitus:0.35), (EMTRICITABINE\RILPIVIRINE\TENOFOVIR DISOPROXIL FUMARATE:Diabetes mellitus:0.03), (EMU OIL:Diabetes mellitus:0.02), (ENFUVIRTIDE:Diabetes mellitus:0.13), (EPACADOSTAT:Diabetes mellitus:0.55), (ESCARGOT:Diabetes mellitus:0.14), (ESCHERICHIA COLI:Diabetes mellitus:1.04), (ESOMEPRAZOLE STRONTIUM:Diabetes mellitus:0.61), (ESSENTIAL OILS:Diabetes mellitus:0.05), (ESTRADIOL BENZOATE:Diabetes mellitus:1.27), (ESTRADIOL CYPIONATE:Diabetes mellitus:1.08), (ESTRADIOL\ESTRIOL:Diabetes mellitus:0.23), (ESTRADIOL\LEVONORGESTREL:Diabetes mellitus:0.63), (ESTRADIOL\PROGESTERONE:Diabetes mellitus:0.38), (ESTRAMUSTINE:Diabetes mellitus:0.36), (ESTRAMUSTINE PHOSPHATE:Diabetes mellitus:0.04), (ESTROGENS, CONJUGATED SYNTHETIC B:Diabetes mellitus:0.49), (ESTROGENS, ESTERIFIED:Diabetes mellitus:0.59), (ESTRONE:Diabetes mellitus:0.39), (ETELCALCETIDE HYDROCHLORIDE:Diabetes mellitus:0.61), (ETEPLIRSEN:Diabetes mellitus:0.05), (ETHINYL ESTRADIOL\FERROUS FUMARATE\NORETHINDRONE:Diabetes mellitus:0.49), (ETHOSUXIMIDE:Diabetes mellitus:0.26), (EZOGABINE:Diabetes mellitus:0.02), (FALDAPREVIR:Diabetes mellitus:0.44), (FENOPROFEN CALCIUM:Diabetes mellitus:0.75), (FERRITIN:Diabetes mellitus:1.23), (FERROUS SUCCINATE:Diabetes mellitus:0.31), (FERUMOXYTOL:Diabetes mellitus:0.24), (FESOTERODINE:Diabetes mellitus:1.7), (FLOMOXEF:Diabetes mellitus:1.12), (FLORBETAPIR F-18:Diabetes mellitus:0.07), (FLUPENTIXOL DIHYDROCHLORIDE:Diabetes mellitus:0.2), (FLUPHENAZINE DECANOATE:Diabetes mellitus:0.27), (FLUPREDNIDENE ACETATE\MICONAZOLE NITRATE:Diabetes mellitus:0.18), (FOLIC ACID\IRON\MINERALS\VITAMINS:Diabetes mellitus:0.63), (FORMALDEHYDE:Diabetes mellitus:0.1), (FOSBRETABULIN TROMETHAMINE:Diabetes mellitus:0.61), (FROVATRIPTAN SUCCINATE:Diabetes mellitus:1.77), (FUROSEMIDE SODIUM\SPIRONOLACTONE:Diabetes mellitus:0.04), (GADOBUTROL:Diabetes mellitus:0.5), (GADOLINIUM:Diabetes mellitus:0.86), (GARLIC OIL:Diabetes mellitus:0.89), (GELATIN:Diabetes mellitus:0.9), (GENISTEIN:Diabetes mellitus:0.39), (GENTIAN VIOLET:Diabetes mellitus:1.11), (GILTERITINIB:Diabetes mellitus:0.1), (GLATIRAMER:Diabetes mellitus:0.73), (GLUCONATE:Diabetes mellitus:1.15), (GLYCERIN\HYPROMELLOSE 2910 (15 MPA.S)\POLYETHYLENE GLYCOL 400\PROPYLENE GLYCOL:Diabetes mellitus:0.33), (GOLD SODIUM THIOMALATE:Diabetes mellitus:0.42), (GUAIAZULENE:Diabetes mellitus:0.55), (HALCINONIDE:Diabetes mellitus:0.96), (HAWTHORN LEAF WITH FLOWER:Diabetes mellitus:0.04), (HEPTAMINOL:Diabetes mellitus:0.27), (HERBALS\HOMEOPATHICS\GOLDENSEAL:Diabetes mellitus:0.04), (HERBALS\PLANTAGO OVATA LEAF:Diabetes mellitus:0.22), (HERBALS\VITIS VINIFERA SEED:Diabetes mellitus:0.42), (HEXACHLOROPHENE:Diabetes mellitus:0.01), (HOMATROPINE\HOMATROPINE HYDROBROMIDE:Diabetes mellitus:0.07), (HUMAN COLOSTRUM:Diabetes mellitus:0.42), (HYALURONIDASE RECOMBINANT HUMAN:Diabetes mellitus:0.02), (HYDROCHLORIC ACID:Diabetes mellitus:1.08), (HYDROCODONE BITARTRATE\PHENYLPROPANOLAMINE HYDROCHLORIDE:Diabetes mellitus:0.13), (HYDROCORTISONE HEMISUCCINATE:Diabetes mellitus:0.57), (HYDROCORTISONE PROBUTATE:Diabetes mellitus:0.87), (HYDROCORTISONE\NEOMYCIN SULFATE\POLYMYXIN B SULFATE:Diabetes mellitus:0.07), (HYDROQUINONE:Diabetes mellitus:0.96), (HYPERICUM PERFORATUM:Diabetes mellitus:0.25), (IBUPROFEN SODIUM:Diabetes mellitus:0.04), (IGURATIMOD:Diabetes mellitus:1.93), (IMIPRAMINE PAMOATE:Diabetes mellitus:0.58), (INDIGOTINDISULFONATE SODIUM:Diabetes mellitus:0.43), (INDINAVIR:Diabetes mellitus:0.09), (INDISETRON HYDROCHLORIDE:Diabetes mellitus:0.2), (INDORAMIN:Diabetes mellitus:0.5), (INFLUENZA A VIRUS A/CALIFORNIA/7/2009 X-179A (H1N1) ANTIGEN (FORMALDEHYDE INACTIVATED)\INFLUENZA A VIRUS A/TEXAS/50/2012 X-223A (H3N2) ANTIGEN (FORMALDEHYDE INACTIVATED)\INFLUENZA B VIRUS B/BRISBANE/60/2008 ANTIGEN (FORMALDEHYDE INACTIVATED)\INFLUENZA B VIRUS B/MASSACHUSETTS/2/2012 ANTIGEN (FORMALDE:Diabetes mellitus:0.1), (INFLUENZA VIRUS VACCINE TRIVALENT TYPE A+B AFLURIA:Diabetes mellitus:1.05), (INOSINE PRANOBEX:Diabetes mellitus:0.23), (INTERFERON ALFA-2A OR INTERFERON ALFA-2B:Diabetes mellitus:1.l), (INTERFERON ALFA-N1:Diabetes mellitus:0.76), (INTERFERON ALFACON-1:Diabetes mellitus:0.54), (INTERFERON GAMMA-1B:Diabetes mellitus:1.91), (IODINE\POTASSIUM IODIDE:Diabetes mellitus:0.85), (IPATASERTIB:Diabetes mellitus:0.27), (ISOPROTERENOL HYDROCHLORIDE:Diabetes mellitus:0.32), (IVY EXTRACT:Diabetes mellitus:0.17), (IXABEPILONE:Diabetes mellitus:0.56), (KONJAC MANNAN:Diabetes mellitus:0.25), (LACTOBACILLUS REUTERI:Diabetes mellitus:0.02), (LANOLIN\MINERAL OIL:Diabetes mellitus:0.08), (LANOLIN\MINERAL OIL\PETROLATUM:Diabetes mellitus:0.8), (LANOLIN\PETROLATUM:Diabetes mellitus:0.71), (LARONIDASE:Diabetes mellitus:0.5), (LATANOPROST\TIMOLOL:Diabetes mellitus:0.8), (LAVENDER OIL:Diabetes mellitus:0.32), (LECITHIN\SODIUM BICARBONATE\SODIUM PHOSPHATE, MONOBASIC:Diabetes mellitus:0.88), (LESINURAD:Diabetes mellitus:0.96), (LETROZOLE\RIBOCICLIB:Diabetes mellitus:0.48), (LEVOBUPIVACAINE HYDROCHLORIDE:Diabetes mellitus:0.28), (LEVOMEFOLATE CALCIUM\METHYLCOBALAMIN\PYRIDOXAL 5-PHOSPHATE:Diabetes mellitus:0.26), (LEVOMEPROMAZINE HYDROCHLORIDE\LEVOMEPROMAZINE MALEATE:Diabetes mellitus:0.3), (LEVORPHANOL TARTRATE:Diabetes mellitus:0.48), (LEVOTHYROXINE SODIUM\LIOTHYRONINE SODIUM:Diabetes mellitus:0.28), (LICORICE ROOT EXTRACT\RHUBARB:Diabetes mellitus:0.61), (LIDOCAINE\TRIBENOSIDE:Diabetes mellitus:0.55), (LINCOMYCIN:Diabetes mellitus:0.39), (LITHIUM SULFATE:Diabetes mellitus:0.14), (LOMERIZINE HYDROCHLORIDE:Diabetes mellitus:0.25), (LUTEINIZING HORMONE:Diabetes mellitus:0.21), (LYNESTRENOL:Diabetes mellitus:0.42), (MAGNESIUM ACETATE:Diabetes mellitus:0.43), (MAGNESIUM GLUCEPTATE:Diabetes mellitus:0.21), (MAGNESIUM STEARATE:Diabetes mellitus:0.24), (MAPROTILINE:Diabetes mellitus:0.33), (MARIZOMIB:Diabetes mellitus:0.09), (MATRICARIA RECUTITA:Diabetes mellitus:0.01), (MEBENDAZOLE:Diabetes mellitus:0.24), (MECHLORETHAMINE:Diabetes mellitus:0.08), (MECLOZINE DIHYDROCHLORIDE:Diabetes mellitus:1.27), (MELDONIUM:Diabetes mellitus:0.49), (MEPENZOLATE BROMIDE:Diabetes mellitus:1.78), (METHENAMINE MANDELATE:Diabetes mellitus:1.99), (METHENOLONE ACETATE:Diabetes mellitus:0.49), (METHSUXIMIDE:Diabetes mellitus:0.07), (METHYLPREDNISOLONE OR METHYLPREDNISOLONE ACETATE:Diabetes mellitus:0.46), (METHYSERGIDE MALEATE:Diabetes mellitus:0.29), (MEXAZOLAM:Diabetes mellitus:0.29), (MIGLUSTAT:Diabetes mellitus:1.67), (MITOMYCIN:Diabetes mellitus:0.05), (NADH:Diabetes mellitus:0.14), (NAFRONYL OXALATE:Diabetes mellitus:0.18), (NALDEMEDINE:Diabetes mellitus:0.95), (NALOXEGOL:Diabetes mellitus:0.13), (NAPROXEN SODIUM\SUMATRIPTAN SUCCINATE:Diabetes mellitus:1.29), (NATURAL LATEX RUBBER:Diabetes mellitus:0.39), (NEISSERIA MENINGITIDIS GROUP A CAPSULAR POLYSACCHARIDE DIPHTHERIA TOXOID CONJUGATE ANTIGEN\NEISSERIA MENINGITIDIS GROUP C CAPSULAR POLYSACCHARIDE DIPHTHERIA TOXOID CONJUGATE ANTIGEN\NEISSERIA MENINGITIDIS GROUP W-135 CAPSULAR POLYSACCHARIDE DIPHTHERIA TOXOID CONJUGATE ANTIGEN\NEISSERIA MENINGITIDIS:Diabetes mellitus:0.29), (NELARABINE:Diabetes mellitus:0.86), (NEOSTIGMINE BROMIDE:Diabetes mellitus:1.02), (NERATINIB:Diabetes mellitus:0.03), (NIFURTOINOL:Diabetes mellitus:0.72), (NILVADIPINE:Diabetes mellitus:0.97), (NIMUSTINE:Diabetes mellitus:0.1), (NITROGEN:Diabetes mellitus:0.78), (NUSINERSEN:Diabetes mellitus:0.48), (OCTOTIAMINE:Diabetes mellitus:0.65), (OK-432:Diabetes mellitus:0.7), (OLIVE OIL:Diabetes mellitus:0.63), (OLODATEROL:Diabetes mellitus:0.65), (OMACETAXINE:Diabetes mellitus:0.54), (OMEGA-6 FATTY ACIDS:Diabetes mellitus:0.19), (OPICAPONE:Diabetes mellitus:0.21), (OPRELVEKIN:Diabetes mellitus:0.25), (ORGOTEIN:Diabetes mellitus:0.29), (ORNITHINE:Diabetes mellitus:0.3), (ORTERONEL:Diabetes mellitus:0.18), (OSTREA EDULIS SHELL:Diabetes mellitus:1.17), (OXANDROLONE:Diabetes mellitus:1.3), (OXETORONE:Diabetes mellitus:0.55), (OXICONAZOLE NITRATE:Diabetes mellitus:0.09), (OZAGREL:Diabetes mellitus:0.9), (PANCRELIPASE\TYROSINE:Diabetes mellitus:0.41), (PANCURONIUM:Diabetes mellitus:0.69), (PANTHENOL:Diabetes mellitus:0.69), (PANTOTHENIC ACID:Diabetes mellitus:0.24), (PAPAYA:Diabetes mellitus:0.97), (PARAFFIN:Diabetes mellitus:0.78), (PARECOXIB SODIUM:Diabetes mellitus:0.59), (PARSLEY:Diabetes mellitus:0.66), (PASCOPYRUM SMITHII POLLEN:Diabetes mellitus:0.45), (PEGINTERFERON ALFA-2B\RIBAVIRIN:Diabetes mellitus:0.11), (PEGINTERFERON LAMBDA-1A:Diabetes mellitus:0.53), (PENBUTOLOL SULFATE:Diabetes mellitus:0.28), (PENCICLOVIR:Diabetes mellitus:0.36), (PENICILLIN V BENZATHINE:Diabetes mellitus:1.19), (PENTETRAZOL:Diabetes mellitus:0.11), (PENTOBARBITAL:Diabetes mellitus:0.46), (PENTOSTATIN:Diabetes mellitus:1.98), (PENTOXYVERINE:Diabetes mellitus:0.02), (PENTOXYVERINE CITRATE:Diabetes mellitus:0.42), (PERAZINE:Diabetes mellitus:0.17), (PERIFOSINE:Diabetes mellitus:0.29), (PHENAZEPAM:Diabetes mellitus:0.48), (PHENELZINE:Diabetes mellitus:0.65), (PHENOBARBITAL SODIUM:Diabetes mellitus:1.31), (PHENOTHIAZINE:Diabetes mellitus:1.12), (PHENYLEPHRINE HYDROCHLORIDE\WITCH HAZEL:Diabetes mellitus:1.94), (PHOSPHATIDYL SERINE:Diabetes mellitus:0.03), (PILSICAINIDE:Diabetes mellitus:0.97), (PIPOBROMAN:Diabetes mellitus:0.41), (PIRARUBICIN:Diabetes mellitus:0.15), (PIRIBEDIL:Diabetes mellitus:0.59), (PIRMENOL HYDROCHLORIDE:Diabetes mellitus:0.75), (PLATINUM:Diabetes mellitus:0.03), (POLIDOCANOL:Diabetes mellitus:0.42), (POLOXAMER:Diabetes mellitus:0.2), (POLYCARBOPHIL:Diabetes mellitus:1.43), (POLYETHYLENE GLYCOL 3350\POTASSIUM CHLORIDE\SODIUM BICARBONATE\SODIUM CHLORIDE\SODIUM SULFATE\SODIUM SULFATE ANHYDROUS:Diabetes mellitus:0.25), (POLYETHYLENE GLYCOL 400:Diabetes mellitus:0.84), (POLYMYXIN:Diabetes mellitus:0.19), (PORFIMER SODIUM:Diabetes mellitus:0.77), (POTASSIUM BICARBONATE\POTASSIUM CHLORIDE:Diabetes mellitus:0.33), (POTASSIUM BICARBONATE\POTASSIUM CITRATE:Diabetes mellitus:0.24), (POTASSIUM BITARTRATE:Diabetes mellitus:0.3), (POTASSIUM BROMIDE:Diabetes mellitus:0.32), (POTASSIUM CHLORIDE\SODIUM CHLORIDE:Diabetes mellitus:0.91), (POTASSIUM SORBATE:Diabetes mellitus:0.29), (POTASSIUM SULFATE:Diabetes mellitus:0.16), (PRAMOXINE:Diabetes mellitus:0.08), (PREDNISOLONE SODIUM METAZOATE:Diabetes mellitus:0.77), (PREDNISONE ACETATE:Diabetes mellitus:0.79), (PREGNENOLONE:Diabetes mellitus:1.14), (PROCARBAZINE:Diabetes mellitus:0.17), (PROMESTRIENE:Diabetes mellitus:0.08), (PROPANTHELINE BROMIDE:Diabetes mellitus:0.7), (PROPOXYPHENE:Diabetes mellitus:0.93), (PSORALEN:Diabetes mellitus:0.89), (PYRIDOXAL PHOSPHATE:Diabetes mellitus:1.58), (PYRIDOXINE\THIAMINE:Diabetes mellitus:0.12), (PYRIMETHAMINE:Diabetes mellitus:1.23), (QUINAGOLIDE HYDROCHLORIDE:Diabetes mellitus:1.25), (RADIUM:Diabetes mellitus:0.01), (RALTITREXED:Diabetes mellitus:0.26), (RANIMUSTINE:Diabetes mellitus:0.22), (RASPBERRY KETONE:Diabetes mellitus:0.12), (RED CLOVER:Diabetes mellitus:0.65), (RED YEAST:Diabetes mellitus:0.73), (REISHI:Diabetes mellitus:0.05), (RESLIZUMAB:Diabetes mellitus:0.27), (RHUBARB:Diabetes mellitus:1.8), (RILMAZAFONE:Diabetes mellitus:0.09), (RILOTUMUMAB:Diabetes mellitus:0.27), (RIMABOTULINUMTOXINB:Diabetes mellitus:0.39), (RIMANTADINE HYDROCHLORIDE:Diabetes mellitus:1.18), (RIPASUDIL HYDROCHLORIDE DIHYDRATE:Diabetes mellitus:0.24), (RISEDRONATE SODIUM HEMI-PENTAHYDRATE:Diabetes mellitus:0.53), (ROLAPITANT:Diabetes mellitus:0.06), (RUFINAMIDE:Diabetes mellitus:1), (RUXOLITINIB PHOSPHATE:Diabetes mellitus:0.25), (SAFINAMIDE:Diabetes mellitus:0.12), (SAQUINAVIR:Diabetes mellitus:0.54), (SARGRAMOSTIM:Diabetes mellitus:0.78), (SARPOGRELATE HYDROCHLORIDE:Diabetes mellitus:1.95), (SEA WATER:Diabetes mellitus:0.82), (SENNOSIDE A:Diabetes mellitus:0.46), (SENNOSIDE A\SENNOSIDE B\SENNOSIDES:Diabetes mellitus:0.57), (SEROTONIN:Diabetes mellitus:0.29), (SERTINDOLE:Diabetes mellitus:0.5), (SHARK CARTILAGE\SHARK, UNSPECIFIED:Diabetes mellitus:0.49), (SHARK LIVER OIL:Diabetes mellitus:0.59), (SILTUXIMAB:Diabetes mellitus:0.2), (SILVER:Diabetes mellitus:0.26), (SILVER NITRATE:Diabetes mellitus:0.94), (SIMEPREVIR SODIUM:Diabetes mellitus:0.18), (SIRUKUMAB:Diabetes mellitus:0.02), (SIVELESTAT SODIUM:Diabetes mellitus:1.17), (SODIUM FLUORIDE\TRICLOSAN:Diabetes mellitus:0), (SODIUM FLUORIDE\VITAMINS:Diabetes mellitus:0.22), (SODIUM HYDROXIDE:Diabetes mellitus:0.11), (SODIUM HYPOCHLORITE:Diabetes mellitus:0.37), (SODIUM PHOSPHATE, DIBASIC:Diabetes mellitus:1.63), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Diabetes mellitus:0.81), (SODIUM PHOSPHATE, DIBASIC, ANHYDROUS\SODIUM PHOSPHATE, MONOBASIC, MONOHYDRATE:Diabetes mellitus:0.48), (SOMATOSTATIN:Diabetes mellitus:0.82), (SOMATREM:Diabetes mellitus:0.47), (SOY ISOFLAVONES:Diabetes mellitus:0.09), (SPIRULINA:Diabetes mellitus:1.08), (STANNOUS FLUORIDE:Diabetes mellitus:0), (STREPTOKINASE:Diabetes mellitus:0.26), (STREPTOMYCIN SULFATE:Diabetes mellitus:0.86), (STREPTOZOCIN:Diabetes mellitus:0.34), (STRONTIUM CHLORIDE:Diabetes mellitus:0), (SULBACTAM SODIUM:Diabetes mellitus:0.03), (SULBUTIAMINE:Diabetes mellitus:0.45), (SULFACETAMIDE SODIUM:Diabetes mellitus:0.77), (SULFACETAMIDE SODIUM\SULFUR:Diabetes mellitus:0.98), (SULFAMERAZINE\TRIMETHOPRIM:Diabetes mellitus:0.76), (SULFAMETHAZINE:Diabetes mellitus:0.5), (SULTOPRIDE HYDROCHLORIDE:Diabetes mellitus:1.23), (SUNITINIB:Diabetes mellitus:0.91), (TANDOSPIRONE CITRATE:Diabetes mellitus:0.11), (TAR:Diabetes mellitus:0.12), (TASELISIB:Diabetes mellitus:0.04), (TECELEUKIN:Diabetes mellitus:0.35), (TELITHROMYCIN:Diabetes mellitus:0.33), (TEMOCAPRIL:Diabetes mellitus:0.73), (TERLIPRESSIN:Diabetes mellitus:0.07), (TETRACAINE HYDROCHLORIDE:Diabetes mellitus:0.69), (TETRACAINE\TETRACAINE HYDROCHLORIDE:Diabetes mellitus:0.45), (TETRAZEPAM:Diabetes mellitus:1.15), (THIAMYLAL SODIUM:Diabetes mellitus:0.76), (THIOGUANINE:Diabetes mellitus:0.32), (THYMOL:Diabetes mellitus:0.9), (TIARAMIDE:Diabetes mellitus:1.26), (TILIDINE HYDROCHLORIDE:Diabetes mellitus:1.35), (TIROFIBAN HYDROCHLORIDE:Diabetes mellitus:0.19), (TOBRAMYCIN SULFATE:Diabetes mellitus:0.43), (TOLCAPONE:Diabetes mellitus:0.26), (TOLMETIN SODIUM:Diabetes mellitus:0.49), (TOLPERISONE:Diabetes mellitus:0.22), (TOLPERISONE HYDROCHLORIDE:Diabetes mellitus:0.34), (TOREMIFENE:Diabetes mellitus:0.27), (TRAPIDIL:Diabetes mellitus:0.38), (TRIBENOSIDE:Diabetes mellitus:0.18), (TRICAPRYLIN:Diabetes mellitus:0.37), (TRICLOSAN:Diabetes mellitus:0.43), (TRIFLUOPERAZINE\TRIFLUOPERAZINE HYDROCHLORIDE:Diabetes mellitus:0.4), (TROLAMINE:Diabetes mellitus:0.34), (TRYPSIN:Diabetes mellitus:0.06), (TRYPTOPHAN:Diabetes mellitus:0.84), (TULOBUTEROL HYDROCHLORIDE:Diabetes mellitus:0.72), (UBIQUINOL:Diabetes mellitus:1.38), (UNDECYLENIC ACID:Diabetes mellitus:0.5), (URACIL:Diabetes mellitus:0.97), (VALRUBICIN:Diabetes mellitus:0.4), (VINBLASTINE SULFATE:Diabetes mellitus:0.61), (VITAMIN A PALMITATE:Diabetes mellitus:0.95), (WHEAT GERM OIL:Diabetes mellitus:0.34), (XYLITOL:Diabetes mellitus:0.38), (XYLOMETAZOLINE:Diabetes mellitus:0.8), (ZANAMIVIR:Diabetes mellitus:0.65), (ZINC ACETATE:Diabetes mellitus:0.7), (ZINC CITRATE:Diabetes mellitus:1.08), (AMIODARONE HYDROCHLORIDE:Diabetes mellitus inadequate control:0.34), (BENAZEPRIL HYDROCHLORIDE:Diabetes mellitus inadequate control:0.53), (BISOPROLOL FUMARATE:Diabetes mellitus inadequate control:0.52), (CALCITRIOL:Diabetes mellitus inadequate control:0.31), (CALCIUM CARBONATE\CHOLECALCIFEROL:Diabetes mellitus inadequate control:0.45), (CHOLESTYRAMINE:Diabetes mellitus inadequate control:0.15), (CITALOPRAM HYDROBROMIDE:Diabetes mellitus inadequate control:0.23), (DIPHENHYDRAMINE:Diabetes mellitus inadequate control:0.18), (ENOXAPARIN:Diabetes mellitus inadequate control:0.16), (EVOLOCUMAB:Diabetes mellitus inadequate control:1.09), (FERROUS SULFATE\FERROUS SULFATE, DRIED:Diabetes mellitus inadequate control:0.53), (IBRUTINIB:Diabetes mellitus inadequate control:0.36), (LERCANIDIPINE:Diabetes mellitus inadequate control:0.01), (LIDOCAINE:Diabetes mellitus inadequate control:0.06), (NEBIVOLOL:Diabetes mellitus inadequate control:0.2), (NILOTINIB:Diabetes mellitus inadequate control:0.06), (POLYETHYLENE GLYCOLS:Diabetes mellitus inadequate control:0.15), (RANOLAZINE:Diabetes mellitus inadequate control:1.16), (SACUBITRIL\VALSARTAN:Diabetes mellitus inadequate control:0.11), (SOTALOL:Diabetes mellitus inadequate control:0.04), (TERIFLUNOMIDE:Diabetes mellitus inadequate control:0.11), (TOPIRAMATE:Diabetes mellitus inadequate control:0.21), (AMIODARONE HYDROCHLORIDE:Diabetes mellitus management:0.46), (BENAZEPRIL HYDROCHLORIDE:Diabetes mellitus management:0.29), (CALCIUM CARBONATE\CHOLECALCIFEROL:Diabetes mellitus management:0.57), (CANDESARTAN CILEXETIL\HYDROCHLOROTHIAZIDE:Diabetes mellitus management:0.25), (CERTOLIZUMAB PEGOL:Diabetes mellitus management:0.15), (CORTISONE\HYDROCORTISONE:Diabetes mellitus management:0.1), (DASATINIB:Diabetes mellitus management:0.02), (DEXLANSOPRAZOLE:Diabetes mellitus management:0.53), (DICLOFENAC:Diabetes mellitus management:0.07), (DOCUSATE SODIUM:Diabetes mellitus management:0.42), (EMPAGLIFLOZIN\METFORMIN HYDROCHLORIDE:Diabetes mellitus management:0.3), (EVOLOCUMAB:Diabetes mellitus management:1.12), (FLECAINIDE:Diabetes mellitus management:0.13), (HYDROXYCHLOROQUINE SULFATE:Diabetes mellitus management:0.25), (LABETALOL\LABETALOL HYDROCHLORIDE:Diabetes mellitus management:0.18), (LATANOPROST:Diabetes mellitus management:0.13), (LERCANIDIPINE:Diabetes mellitus management:0.77), (METRONIDAZOLE:Diabetes mellitus management:0.06), (NEBIVOLOL:Diabetes mellitus management:0.01), (OXYGEN:Diabetes mellitus management:0.15), (SECUKINUMAB:Diabetes mellitus management:0.12), (SODIUM BICARBONATE:Diabetes mellitus management:0.44), (TERIFLUNOMIDE:Diabetes mellitus management:0.45), (POLYMYXIN B SULFATE\TRIMETHOPRIM SULFATE:Diabetic eye disease:0.24), (BUDESONIDE:Diabetic foot:0.72), (CANDESARTAN:Diabetic foot:0.14), (CANDESARTAN CILEXETIL:Diabetic foot:0.13), (CETIRIZINE HYDROCHLORIDE:Diabetic foot:0.18), (CYCLOBENZAPRINE:Diabetic foot:0.2), (DEVICE:Diabetic foot:0.51), (DILTIAZEM HYDROCHLORIDE:Diabetic foot:0.7), (ENOXAPARIN:Diabetic foot:0.37), (FLECAINIDE:Diabetic foot:0.05), (FLUTICASONE PROPIONATE:Diabetic foot:0.74), (IBRUTINIB:Diabetic foot:0.25), (IRBESARTAN:Diabetic foot:0.15), (METHYLPREDNISOLONE SODIUM SUCCINATE:Diabetic foot:0.01), (NAPROXEN:Diabetic foot:0.73), (OXCARBAZEPINE:Diabetic foot:0.11), (RANITIDINE HYDROCHLORIDE:Diabetic foot:0.19), (VERAPAMIL HYDROCHLORIDE:Diabetic foot:0.41), (BENAZEPRIL HYDROCHLORIDE:Diabetic nephropathy:0.29), (CANDESARTAN CILEXETIL\HYDROCHLOROTHIAZIDE:Diabetic nephropathy:0.19), (CARVEDILOL PHOSPHATE:Diabetic nephropathy:0.07), (CERTOLIZUMAB PEGOL:Diabetic nephropathy:0.27), (CHOLESTYRAMINE:Diabetic nephropathy:0.13), (CLOTRIMAZOLE:Diabetic nephropathy:0.04), (DASATINIB:Diabetic nephropathy:0.16), (DEXLANSOPRAZOLE:Diabetic nephropathy:0.64), (DICLOFENAC:Diabetic nephropathy:0.23), (DILTIAZEM:Diabetic nephropathy:0.43), (DIPHENHYDRAMINE:Diabetic nephropathy:0.11), (EDOXABAN TOSYLATE:Diabetic nephropathy:0.37), (ENOXAPARIN:Diabetic nephropathy:0.6), (EVOLOCUMAB:Diabetic nephropathy:1.05), (FERRIC HYDROXIDE:Diabetic nephropathy:0.27), (FLECAINIDE:Diabetic nephropathy:0.05), (IBRUTINIB:Diabetic nephropathy:0.73), (ILOPROST:Diabetic nephropathy:0.06), (LABETALOL\LABETALOL HYDROCHLORIDE:Diabetic nephropathy:0.15), (METRONIDAZOLE:Diabetic nephropathy:0.47), (NEBIVOLOL:Diabetic nephropathy:0.29), (NILOTINIB:Diabetic nephropathy:0.15), (OMEGA-3-ACID ETHYL ESTERS:Diabetic nephropathy:0.33), (PARICALCITOL:Diabetic nephropathy:0.18), (PILSICAINIDE HYDROCHLORIDE:Diabetic nephropathy:0.09), (POLYETHYLENE GLYCOL 3350:Diabetic nephropathy:0.17), (POLYETHYLENE GLYCOLS:Diabetic nephropathy:0.39), (PRIMIDONE:Diabetic nephropathy:0.2), (RANOLAZINE:Diabetic nephropathy:1.26), (SACUBITRIL\VALSARTAN:Diabetic nephropathy:0.33), (TERIFLUNOMIDE:Diabetic nephropathy:0.39), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Diabetic neuropathy:0.36), (ABIRATERONE ACETATE:Diabetic neuropathy:0.71), (ACETAMINOPHEN\CAFFEINE:Diabetic neuropathy:0.14), (ADO-TRASTUZUMAB EMTANSINE:Diabetic neuropathy:0.05), (AGALSIDASE BETA:Diabetic neuropathy:0.82), (ALCOHOL:Diabetic neuropathy:0.5), (ALENDRONIC ACID:Diabetic neuropathy:0.65), (ALISKIREN:Diabetic neuropathy:1.09), (ALUMINUM HYDROXIDE\DIMETHICONE\MAGNESIUM HYDROXIDE:Diabetic neuropathy:0.49), (AMINO ACIDS\DEXTROSE\ELECTROLYTES NOS:Diabetic neuropathy:0.04), (AMINO ACIDS\DEXTROSE\ELECTROLYTES NOS\SOYBEAN OIL\VITAMINS:Diabetic neuropathy:0.22), (AMISULPRIDE:Diabetic neuropathy:0.41), (AMLODIPINE BESYLATE\TELMISARTAN:Diabetic neuropathy:0.12), (AMLODIPINE\VALSARTAN:Diabetic neuropathy:0.64), (AMMONIUM CHLORIDE\DEXTROMETHORPHAN\SODIUM CITRATE:Diabetic neuropathy:0.12), (AMOXICILLIN\CLARITHROMYCIN\ESOMEPRAZOLE:Diabetic neuropathy:0.16), (AMOXICILLIN\CLAVULANIC ACID:Diabetic neuropathy:0.03), (ANTIHISTAMINES NOS:Diabetic neuropathy:0.06), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Diabetic neuropathy:0.02), (BECLOMETHASONE:Diabetic neuropathy:0.05), (BEPRIDIL:Diabetic neuropathy:0.3), (BETAMETHASONE\CALCIPOTRIENE:Diabetic neuropathy:0.17), (BETHANECHOL CHLORIDE:Diabetic neuropathy:0.18), (BIFIDOBACTERIUM SPP.:Diabetic neuropathy:0.05), (CALCITONIN SALMON:Diabetic neuropathy:0.05), (CALCIUM CARBONATE\VITAMIN D:Diabetic neuropathy:0.46), (CALCIUM\MAGNESIUM:Diabetic neuropathy:0.67), (CEFAZOLIN:Diabetic neuropathy:0.06), (CEFUROXIME AXETIL:Diabetic neuropathy:0.16), (CEVIMELINE HYDROCHLORIDE:Diabetic neuropathy:0.14), (CHLORPROMAZINE:Diabetic neuropathy:0.03), (CICLESONIDE:Diabetic neuropathy:0.68), (CILASTATIN SODIUM\IMIPENEM:Diabetic neuropathy:0.21), (CLOMIPRAMINE HYDROCHLORIDE:Diabetic neuropathy:0.14), (COD LIVER OIL:Diabetic neuropathy:0.53), (CORTICOTROPIN:Diabetic neuropathy:0.95), (DABIGATRAN:Diabetic neuropathy:1.31), (DACLIZUMAB:Diabetic neuropathy:1.16), (DALTEPARIN SODIUM:Diabetic neuropathy:0.11), (DANAZOL:Diabetic neuropathy:0.3), (DEFLAZACORT:Diabetic neuropathy:1.16), (DEGARELIX:Diabetic neuropathy:0.09), (DEXAMETHASONE ACETATE:Diabetic neuropathy:0.23), (DEXTROAMPHETAMINE SULFATE:Diabetic neuropathy:0.61), (DIGITOXIN:Diabetic neuropathy:0.38), (DIPYRIDAMOLE:Diabetic neuropathy:0.96), (DOFETILIDE:Diabetic neuropathy:1.61), (DORZOLAMIDE HYDROCHLORIDE:Diabetic neuropathy:0.22), (DRONABINOL:Diabetic neuropathy:1.32), (DUTASTERIDE\TAMSULOSIN HYDROCHLORIDE:Diabetic neuropathy:0.99), (ECONAZOLE:Diabetic neuropathy:0.37), (EDOXABAN TOSYLATE:Diabetic neuropathy:1.81), (ESTRONE\SUS SCROFA OVARY:Diabetic neuropathy:0.74), (ETRETINATE:Diabetic neuropathy:0.01), (EVEROLIMUS:Diabetic neuropathy:1.76), (EXEMESTANE:Diabetic neuropathy:0), (FERRIC CITRATE ANHYDROUS:Diabetic neuropathy:0.15), (FILGRASTIM:Diabetic neuropathy:0.24), (FLUINDIONE:Diabetic neuropathy:0.22), (FLUOROMETHOLONE:Diabetic neuropathy:0.03), (FORMOTEROL:Diabetic neuropathy:0.37), (FORMOTEROL FUMARATE:Diabetic neuropathy:0.78), (FORMOTEROL FUMARATE\MOMETASONE FUROATE:Diabetic neuropathy:0.22), (FURSULTIAMINE:Diabetic neuropathy:0.09), (FUSIDIC ACID:Diabetic neuropathy:0.69), (GLIMEPIRIDE\ROSIGLITAZONE MALEATE:Diabetic neuropathy:0.43), (GLYCOPYRROLATE:Diabetic neuropathy:0.31), (GLYCOPYRROLATE\INDACATEROL MALEATE:Diabetic neuropathy:0.07), (GOSERELIN ACETATE:Diabetic neuropathy:0.12), (GUANFACINE HYDROCHLORIDE:Diabetic neuropathy:0.14), (HEPARIN CALCIUM:Diabetic neuropathy:0.18), (HYDROXYCHLOROQUINE DIPHOSPHATE:Diabetic neuropathy:0.37), (ILOPROST:Diabetic neuropathy:0.85), (INSULIN ASPART\INSULIN DEGLUDEC:Diabetic neuropathy:0.26), (INTERFERON:Diabetic neuropathy:0.72), (INTERFERON ALFA-2A:Diabetic neuropathy:0), (IXAZOMIB:Diabetic neuropathy:0.07), (LACTASE:Diabetic neuropathy:0.38), (LACTOBACILLUS RHAMNOSUS:Diabetic neuropathy:0.4), (LOMITAPIDE MESYLATE:Diabetic neuropathy:1.42), (MEFENAMIC ACID:Diabetic neuropathy:0.18), (MEPOLIZUMAB:Diabetic neuropathy:0.27), (METFORMIN HYDROCHLORIDE\ROSIGLITAZONE MALEATE:Diabetic neuropathy:0.91), (METHENAMINE HIPPURATE:Diabetic neuropathy:1.25), (METHYLPREDNISOLONE ACETATE:Diabetic neuropathy:0.91), (METRELEPTIN:Diabetic neuropathy:0.53), (MOXIFLOXACIN:Diabetic neuropathy:0.09), (MOXIFLOXACIN HYDROCHLORIDE:Diabetic neuropathy:1.72), (MOXONIDINE:Diabetic neuropathy:0.57), (NALOXONE HYDROCHLORIDE\TILIDINE HYDROCHLORIDE:Diabetic neuropathy:0.22), (NALTREXONE:Diabetic neuropathy:0.81), (NINTEDANIB:Diabetic neuropathy:0.87), (NIRAPARIB:Diabetic neuropathy:0.22), (ONABOTULINUMTOXINA:Diabetic neuropathy:0.59), (ORPHENADRINE:Diabetic neuropathy:0.07), (OSELTAMIVIR PHOSPHATE:Diabetic neuropathy:0.16), (OSPEMIFENE:Diabetic neuropathy:0.58), (OXYBUTYNIN\OXYBUTYNIN CHLORIDE:Diabetic neuropathy:0.54), (PARATHYROID HORMONE:Diabetic neuropathy:0.07), (PAZOPANIB HYDROCHLORIDE:Diabetic neuropathy:1.09), (PEMETREXED DISODIUM:Diabetic neuropathy:0.12), (PHENOBARBITAL:Diabetic neuropathy:0.95), (PHYTONADIONE:Diabetic neuropathy:0.18), (PILSICAINIDE HYDROCHLORIDE:Diabetic neuropathy:0.74), (PIMOBENDAN:Diabetic neuropathy:0.26), (PROTRIPTYLINE HYDROCHLORIDE:Diabetic neuropathy:0.28), (REGORAFENIB:Diabetic neuropathy:0.11), (RILPIVIRINE HYDROCHLORIDE:Diabetic neuropathy:0.3), (RIVASTIGMINE TARTRATE:Diabetic neuropathy:0.93), (RIZATRIPTAN:Diabetic neuropathy:0.49), (SALMETEROL:Diabetic neuropathy:0.43), (SELEGILINE:Diabetic neuropathy:0.11), (SODIUM PHOSPHATE, DIBASIC\SODIUM PHOSPHATE, MONOBASIC:Diabetic neuropathy:0.73), (SODIUM PICOSULFATE:Diabetic neuropathy:0.08), (TELOTRISTAT ETHYL:Diabetic neuropathy:0), (TERBUTALINE SULFATE:Diabetic neuropathy:0.83), (TESTOSTERONE UNDECANOATE:Diabetic neuropathy:0.73), (TETRACYCLINE:Diabetic neuropathy:1.31), (THALIDOMIDE:Diabetic neuropathy:0.83), (TOLVAPTAN:Diabetic neuropathy:0), (TROSPIUM CHLORIDE:Diabetic neuropathy:0.57), (UMECLIDINIUM BROMIDE:Diabetic neuropathy:0.52), (VORTIOXETINE HYDROBROMIDE:Diabetic neuropathy:0.48), (YEAST:Diabetic neuropathy:0.33), (ZIPRASIDONE HYDROCHLORIDE\ZIPRASIDONE MESYLATE:Diabetic neuropathy:0.33), (ZOPICLONE:Diabetic neuropathy:1.22), (ALISKIREN:Diabetic retinal oedema:0.26), (AMLODIPINE BESYLATE\HYDROCHLOROTHIAZIDE\OLMESARTAN MEDOXOMIL:Diabetic retinal oedema:0.46), (AMLODIPINE BESYLATE\HYDROCHLOROTHIAZIDE\VALSARTAN:Diabetic retinal oedema:0.09), (BENAZEPRIL\BENAZEPRIL HYDROCHLORIDE:Diabetic retinal oedema:0.04), (BRINZOLAMIDE\TIMOLOL MALEATE:Diabetic retinal oedema:0.16), (CANAGLIFLOZIN\METFORMIN HYDROCHLORIDE:Diabetic retinal oedema:0.52), (CARBOXYMETHYLCELLULOSE SODIUM:Diabetic retinal oedema:0.47), (CARTEOLOL HYDROCHLORIDE:Diabetic retinal oedema:0.69), (CARVEDILOL PHOSPHATE:Diabetic retinal oedema:0.73), (CLOTRIMAZOLE:Diabetic retinal oedema:0.43), (DABIGATRAN:Diabetic retinal oedema:0.42), (DEXAMETHASONE SODIUM PHOSPHATE\\MOXIFLOXACIN HYDROCHLORIDE:Diabetic retinal oedema:0), (DIPHENHYDRAMINE:Diabetic retinal oedema:0.22), (DOFETILIDE:Diabetic retinal oedema:0.51), (DUTASTERIDE:Diabetic retinal oedema:0.82), (EDOXABAN TOSYLATE:Diabetic retinal oedema:0.47), (EMPAGLIFLOZIN\LINAGLIPTIN:Diabetic retinal oedema:0.58), (ESZOPICLONE:Diabetic retinal oedema:0.16), (EVEROLIMUS:Diabetic retinal oedema:0.5), (EVOLOCUMAB:Diabetic retinal oedema:0.8), (FLECAINIDE:Diabetic retinal oedema:0.62), (FLUTICASONE:Diabetic retinal oedema:0.18), (HEPARIN SODIUM:Diabetic retinal oedema:0.69), (HYDROCHLOROTHIAZIDE\IRBESARTAN:Diabetic retinal oedema:0.2), (HYDROCODONE:Diabetic retinal oedema:0.71), (HYDROMORPHONE HYDROCHLORIDE:Diabetic retinal oedema:0.03), (HYOSCYAMINE:Diabetic retinal oedema:0.03), (IBRUTINIB:Diabetic retinal oedema:0.58), (ILOPROST:Diabetic retinal oedema:0.13), (INDAPAMIDE\PERINDOPRIL:Diabetic retinal oedema:0.69), (IPRATROPIUM:Diabetic retinal oedema:0.08), (LEDIPASVIR\SOFOSBUVIR:Diabetic retinal oedema:0.04), (LERCANIDIPINE HYDROCHLORIDE:Diabetic retinal oedema:0.54), (LEVALBUTEROL HYDROCHLORIDE:Diabetic retinal oedema:0.06), (METAXALONE:Diabetic retinal oedema:0.42), (METFORMIN HYDROCHLORIDE\ROSIGLITAZONE MALEATE:Diabetic retinal oedema:0.33), (METHYLPREDNISOLONE SODIUM SUCCINATE:Diabetic retinal oedema:0.36), (NADOLOL:Diabetic retinal oedema:0.19), (OLODATEROL HYDROCHLORIDE\TIOTROPIUM BROMIDE MONOHYDRATE:Diabetic retinal oedema:0.07), (PANTOPRAZOLE MAGNESIUM:Diabetic retinal oedema:0.04), (PAROXETINE\PAROXETINE HYDROCHLORIDE:Diabetic retinal oedema:0.05), (PATIROMER:Diabetic retinal oedema:0.02), (PONATINIB:Diabetic retinal oedema:0.07), (PRAMIPEXOLE:Diabetic retinal oedema:0.13), (PROMETHAZINE\PROMETHAZINE HYDROCHLORIDE:Diabetic retinal oedema:0.33), (RABEPRAZOLE SODIUM:Diabetic retinal oedema:0.08), (RALOXIFENE HYDROCHLORIDE:Diabetic retinal oedema:0.22), (SACUBITRIL\VALSARTAN:Diabetic retinal oedema:0.02), (SODIUM OXYBATE:Diabetic retinal oedema:0.11), (SODIUM PHOSPHATE, DIBASIC\SODIUM PHOSPHATE, MONOBASIC:Diabetic retinal oedema:0.17), (TIMOLOL MALEATE\TRAVOPROST:Diabetic retinal oedema:0.83), (TRAZODONE HYDROCHLORIDE:Diabetic retinal oedema:1.01), (VALGANCICLOVIR HYDROCHLORIDE:Diabetic retinal oedema:0.18), (VITAMIN B:Diabetic retinal oedema:0.41), (BISOPROLOL FUMARATE:Diabetic retinopathy:0.87), (BUDESONIDE:Diabetic retinopathy:0.44), (CALCIUM:Diabetic retinopathy:0.24), (CALCIUM CARBONATE\CHOLECALCIFEROL:Diabetic retinopathy:0.12), (CARTEOLOL HYDROCHLORIDE:Diabetic retinopathy:0.31), (CARVEDILOL PHOSPHATE:Diabetic retinopathy:0.09), (CERTOLIZUMAB PEGOL:Diabetic retinopathy:0.03), (DAPAGLIFLOZIN PROPANEDIOL:Diabetic retinopathy:0.16), (DICLOFENAC:Diabetic retinopathy:0.16), (DUTASTERIDE:Diabetic retinopathy:0.09), (EVOLOCUMAB:Diabetic retinopathy:0.71), (FLECAINIDE:Diabetic retinopathy:0), (FLUOCINOLONE ACETONIDE:Diabetic retinopathy:0.35), (HEPARIN SODIUM:Diabetic retinopathy:0.04), (ISOSORBIDE:Diabetic retinopathy:0.24), (LERCANIDIPINE:Diabetic retinopathy:0.04), (LOTEPREDNOL ETABONATE:Diabetic retinopathy:0.07), (MELOXICAM:Diabetic retinopathy:0.35), (METHYLPREDNISOLONE SODIUM SUCCINATE:Diabetic retinopathy:0.01), (OMEGA-3-ACID ETHYL ESTERS:Diabetic retinopathy:0.32), (OXYGEN:Diabetic retinopathy:0.43), (PHENYLEPHRINE HYDROCHLORIDE\TROPICAMIDE:Diabetic retinopathy:0.77), (POLYETHYLENE GLYCOLS:Diabetic retinopathy:0.05), (SOTALOL:Diabetic retinopathy:0.19), (TAMSULOSIN:Diabetic retinopathy:0.7), (TIMOLOL\TRAVOPROST:Diabetic retinopathy:0.15), (TIOTROPIUM BROMIDE MONOHYDRATE:Diabetic retinopathy:0.01), (TOPIRAMATE:Diabetic retinopathy:0.59), (UNSPECIFIED INGREDIENT:Diabetic retinopathy:0.48), (VERAPAMIL HYDROCHLORIDE:Diabetic retinopathy:0.6), (BISOPROLOL FUMARATE:Diabetic ulcer:0.33), (BUDESONIDE:Diabetic ulcer:0.45), (DAPAGLIFLOZIN:Diabetic ulcer:0.01), (DEVICE:Diabetic ulcer:0.38), (EVOLOCUMAB:Diabetic ulcer:0.7), (FERROUS SULFATE\FERROUS SULFATE, DRIED:Diabetic ulcer:0.2), (IBRUTINIB:Diabetic ulcer:0.02), (NAPROXEN:Diabetic ulcer:0.59), (OMEGA-3-ACID ETHYL ESTERS:Diabetic ulcer:0.04), (OXYGEN:Diabetic ulcer:0.35), (RANITIDINE HYDROCHLORIDE:Diabetic ulcer:0.04), (VERAPAMIL HYDROCHLORIDE:Diabetic ulcer:0.23), (ZOLPIDEM\ZOLPIDEM TARTRATE:Diabetic ulcer:0.05), (ACETAMINOPHEN\BUTALBITAL\CAFFEINE:Diagnostic procedure:0.31), (ALCOHOL:Diagnostic procedure:0.22), (ALISKIREN:Diagnostic procedure:0.23), (AMPHETAMINE ASPARTATE\AMPHETAMINE SULFATE\DEXTROAMPHETAMINE SACCHARATE\DEXTROAMPHETAMINE SULFATE:Diagnostic procedure:0.22), (AMPICILLIN:Diagnostic procedure:0.33), (ATROPINE SULFATE\DIPHENOXYLATE HYDROCHLORIDE:Diagnostic procedure:0.01), (AZITHROMYCIN DIHYDRATE:Diagnostic procedure:0.24), (BENAZEPRIL\BENAZEPRIL HYDROCHLORIDE:Diagnostic procedure:0.06), (BISACODYL OR DOCUSATE SODIUM:Diagnostic procedure:0.56), (BUDESONIDE\FORMOTEROL FUMARATE:Diagnostic procedure:0.22), (BUPRENORPHINE HYDROCHLORIDE\NALOXONE HYDROCHLORIDE:Diagnostic procedure:0.15), (BUSPIRONE HYDROCHLORIDE:Diagnostic procedure:0.19), (CALCIUM CITRATE:Diagnostic procedure:0.03), (CARVEDILOL PHOSPHATE:Diagnostic procedure:0.1), (CERTOLIZUMAB PEGOL:Diagnostic procedure:1.12), (CHOLESTYRAMINE:Diagnostic procedure:0.78), (CILASTATIN SODIUM\IMIPENEM:Diagnostic procedure:0.42), (CLOTRIMAZOLE:Diagnostic procedure:0.42), (CORTISONE\HYDROCORTISONE:Diagnostic procedure:0.37), (DABIGATRAN:Diagnostic procedure:0.76), (DASATINIB:Diagnostic procedure:0.6), (DICYCLOMINE HYDROCHLORIDE:Diagnostic procedure:0.14), (DIGITOXIN:Diagnostic procedure:0.04), (DIMENHYDRINATE:Diagnostic procedure:0.14), (DOFETILIDE:Diagnostic procedure:0.07), (EDOXABAN TOSYLATE:Diagnostic procedure:0.62), (ETORICOXIB:Diagnostic procedure:0.02), (EVEROLIMUS:Diagnostic procedure:0.85), (EVOLOCUMAB:Diagnostic procedure:0.49), (FERROUS SULFATE:Diagnostic procedure:1.23), (FESOTERODINE FUMARATE:Diagnostic procedure:0.07), (FLECAINIDE:Diagnostic procedure:1.13), (FLUTICASONE FUROATE\VILANTEROL TRIFENATATE:Diagnostic procedure:0.38), (FULVESTRANT:Diagnostic procedure:0.04), (GLYCOPYRRONIUM\INDACATEROL:Diagnostic procedure:0.8), (GUAIFENESIN:Diagnostic procedure:0.05), (HYDROCHLOROTHIAZIDE\TELMISARTAN:Diagnostic procedure:0.02), (HYDROCHLOROTHIAZIDE\VALSARTAN:Diagnostic procedure:0.26), (HYDROXYZINE PAMOATE:Diagnostic procedure:0.47), (IDARUCIZUMAB:Diagnostic procedure:0.26), (INFLIXIMAB-DYYB:Diagnostic procedure:0.02), (IPRATROPIUM:Diagnostic procedure:0.77), (ISOSORBIDE:Diagnostic procedure:0.47), (LABETALOL HYDROCHLORIDE:Diagnostic procedure:0.19), (LACOSAMIDE:Diagnostic procedure:0.44), (LEDIPASVIR\SOFOSBUVIR:Diagnostic procedure:0.31), (LIDOCAINE\PRILOCAINE:Diagnostic procedure:0.02), (LITHIUM CARBONATE:Diagnostic procedure:0.67), (METHADONE HYDROCHLORIDE:Diagnostic procedure:0.46), (METHYLPREDNISOLONE ACETATE:Diagnostic procedure:0.24), (NALOXONE HYDROCHLORIDE\OXYCODONE HYDROCHLORIDE:Diagnostic procedure:0.53), (NILOTINIB:Diagnostic procedure:1.32), (NOREPINEPHRINE:Diagnostic procedure:0.13), (OXYGEN:Diagnostic procedure:0.52), (PANTOPRAZOLE MAGNESIUM:Diagnostic procedure:0.82), (PARICALCITOL:Diagnostic procedure:0.2), (PAROXETINE:Diagnostic procedure:0.28), (PEMETREXED DISODIUM:Diagnostic procedure:0.05), (PENICILLIN:Diagnostic procedure:0.35), (PRAVASTATIN:Diagnostic procedure:1.23), (PRIMIDONE:Diagnostic procedure:0.34), (RANOLAZINE:Diagnostic procedure:0.79), (ROFLUMILAST:Diagnostic procedure:0.24), (SACUBITRIL\VALSARTAN:Diagnostic procedure:0.28), (SODIUM OXYBATE:Diagnostic procedure:0.11), (SULFAMETHOXAZOLE:Diagnostic procedure:0.02), (TERBUTALINE:Diagnostic procedure:0.29), (TICLOPIDINE:Diagnostic procedure:0.53), (TINZAPARIN SODIUM:Diagnostic procedure:0.37), (TIZANIDINE HYDROCHLORIDE:Diagnostic procedure:0.16), (TOCILIZUMAB:Diagnostic procedure:0.27), (UMECLIDINIUM BROMIDE\VILANTEROL TRIFENATATE:Diagnostic procedure:0.43), (VALGANCICLOVIR HYDROCHLORIDE:Diagnostic procedure:0.26), (VARENICLINE TARTRATE:Diagnostic procedure:0.2), (BUDESONIDE:Dialysis:0.09), (DILTIAZEM:Dialysis:0.2), (EPINEPHRINE: Dialysis:0.07), (EVEROLIMUS:Dialysis:0.26), (EVOLOCUMAB:Dialysis:0.12), (FERRIC HYDROXIDE:Dialysis:0.02), (FLUTICASONE PROPIONATE:Dialysis:0.48), (LEDIPASVIR\SOFOSBUVIR:Dialysis:0.07), (LOVASTATIN: Dialysis:0.03), (METFORMIN HYDROCHLORIDE:Dialysis:0.34), (OXYGEN:Dialysis:0.14), (PHENOBARBITAL:Dialysis:0.26), (POTASSIUM:Dialysis:0.42), (PYRAZINAMIDE:Dialysis:0.61), (SACUBITRIL\VALSART AN:Dialysis:0.01), (TERIFLUNOMIDE:Dialysis:0.07), (VERAPAMIL HYDROCHLORIDE:Dialysis:0.47), (ZONISAMIDE:Dialysis:0.26), (.ALPHA. 1-PROTEINASE INHIBITOR HUMAN:Diarrhoea:1.91), (ACAMPROSATE:Diarrhoea:0.17), (ACEBUTOLOL HYDROCHLORIDE:Diarrhoea:1.49), (ACETAMINOPHEN\APRONALIDE\CAFFEINE\PROP YPHENAZONE:Diarrhoea:0.61), (ACETAMINOPHEN\BROMISOVAL\DIHYDROCODEINE PHOSPHATE\DIPHENHYDRAMINE SALICYLATE\DYPHYLLINE\METHYLEPHEDRINE HYDROCHLORIDE, (+/−)-:Diarrhoea:0.12), (ACETAMINOPHEN\BUTYLSCOPOLAMINE BROMIDE:Diarrhoea:0), (ACETAMINOPHEN\PROPOXYPHENE HYDROCHLORIDE:Diarrhoea:1.51), (ACETAMINOPHEN\PROPOXYPHENE NAPSYLATE:Diarrhoea:1.38), (ACITRETIN:Diarrhoea:1.51), (ACOTIAMIDE HYDROCHLORIDE:Diarrhoea:0.03), (AJMALINE:Diarrhoea:0.22), (ALBUTEROL\GUAIFEN ESIN:Diarrhoea:0.39), (ALBUTEROL\IPRATROPIUM BROMIDE:Diarrhoea:0.21), (ALISKIREN HEMIFUMARATE\HYDROCHLOROTHIAZIDE:Diarrhoea:0.29), (ALUMINUM HYDROXIDE\DIMETHICONE\DIPHENHYDRAMINE\HYDROXIDE ION\LIDOCAINE:Diarrhoea:0.19), (ALUMINUM HYDROXIDE\MAGNESIUM HYDROXIDE\SIMETHICONE:Diarrhoea:0.34), (ALUMINUM HYDROXIDE\MAGNESIUM OXIDE\OXETHAZAINE:Diarrhoea:1.07), (AMILORIDE HYDROCHLORIDE\FUROSEMIDE:Diarrhoea:0.11), (AMINO ACIDS\VITAMINS:Diarrhoea:0.1), (AMLODIPINE BESYLATE\HYDROCHLOROTHIAZIDE\VALSARTAN:Diarrhoea:1.62), (AMLODIPINE BESYLATE\TELMISARTAN:Diarrhoea:2.01), (AMLODIPINE\VALSARTAN:Diarrhoea:1.81), (AMMONIUM GLYCYRRHIZATE\CYSTEINE HYDROCHLORIDE\GLYCINE:Diarrhoea:1.12), (AMOXAPINE:Diarrhoea:1.21), (AMOXICILLIN SODIUM:Diarrhoea:0.57), (AMOXICILLIN\CLARITHROMYCIN\ESOMEPRAZOLE:Diarrhoea: 1.83), (ANAGLIPTIN:Diarrhoea:0.31), (ANAGRELIDE HYDROCHLORIDE:Diarrhoea:0.92), (APRINDINE HYDROCHLORIDE:Diarrhoea:0.03), (ARIPIPRAZOLE LAUROXIL:Diarrhoea:0.03), (AROTINOLOL:Diarrhoea:0.24), (ARSENIC TRIOXIDE:Diarrhoea:0.23), (ASCORBIC ACID/CHROMIUM/SELENIUM/VANADYL SULFATE:Diarrhoea:0.02), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Diarrhoea:0.91), (ASCORBIC ACID\CALCIUM PANTOTHENATE:Diarrhoea:0.87), (ASCORBIC ACID\VITAMIN B COMPLEX:Diarrhoea:0.31), (ASENAPINE MALEATE:Diarrhoea:0.13), (ASPARAGINASE:Diarrhoea:1.11), (ASPIRIN\CLOPIDOGREL: Diarrhoea:0.76), (ASPIRIN\DIHYDROXYALUMINUM AMINOACETATE ANHYDROUS\MAGNESIUM CARBONATE:Diarrhoea:1.19), (ATRACURIUM:Diarrhoea:0.67), (ATRACURIUM BESYLATE:Diarrhoea:0.63), (AVOBENZONE\HOMOSALATE\OCTISALATE:Diarrhoea:0.33), (AZITHROMYCIN MONOHYDRATE:Diarrhoea:0.24), (AZITHROMYCIN OR METHOTREXATE SODIUM:Diarrhoea:0.74), (BACITRACIN ZINC:Diarrhoea:0.02), (BALSAM PERU OIL\CASTOR OIL\TRYPSIN:Diarrhoea:0.12), (BAMIFYLLINE HYDROCHLORIDE:Diarrhoea:0.41), (BAZEDOXIFENE ACETATE\ESTROGENS, CONJUGATED:Diarrhoea:0.04), (BENIDIPINE:Diarrhoea:0.34), (BENPROPERINE PHOSPHATE:Diarrhoea:0.32), (BEPRIDIL:Diarrhoea:1.28), (BERAPROST:Diarrhoea:0.86), (B ETAMETHASONE DIPROPIONATE\CALCIPOTRIENE:Diarrhoea:1.1), (BETAMETHASONE DIPROPIONATE\CALCIPOTRIENE HYDRATE:Diarrhoea:0.4), (BETAMETHASONE SODIUM PHOSPHATE\NEOMYCIN SULFATE:Diarrhoea:0.76), (BETAMETHASONE\CLOTRIMAZOLE:Diarrhoea:0.1), (BETAM IPRON\PANIPENEM:Diarrhoea:1.31), (BIXALOMER:Diarrhoea:0.18), (BLESELUMAB:Diarrhoea: 0.99), (BLONANSERIN:Diarrhoea:0.45), (BOTULINUM TOXIN TYPE A:Diarrhoea:0.55), (BOTULISM ANTITOXIN:Diarrhoea:0.06), (BRINZOLAMIDE\TIMOLOL MALEATE:Diarrhoea:0.22), (BROMHEXINE:Diarrhoea:0.88), (BUFORMIN:Diarrhoea:0.39), (BUPIVACAINE HYDROCHLORIDE:Diarrhoea:0.81), (CALCIUM BICARBONATE:Diarrhoea:0.21), (CALCIUM CARBONATE\ETIDRONATE DISODIUM:Diarrhoea:0.07), (CALCIUM\MAGNESIUM\ZINC:Diarrhoea:1.44), (CALCIUM\V ITAMINS:Diarrhoea:0.45), (CANAGLIFLOZIN\METFORMIN HYDROCHLORIDE:Diarrhoea:2.11), (CANDESARTAN CILEXETIL\HYDROCHLOROTHIAZIDE:Diarrhoea:3.46), (CANNABIDIOL:Diarrhoea:0.14), (CARBAMIDE PEROXIDE:Diarrhoea:0.36), (CARNITINE:Diarrhoea:0.2), (CARTEOLOL:Diarrhoea:0.17), (CE FOPERAZONE\SULBACTAM:Diarrhoea:0.01), (CEFPIROME SULFATE:Diarrhoea:1), (CEFTAZIDIME SODIUM:Diarrhoea:0.68), (CHLORAL HYDRATE:Diarrhoea:0.19), (CHOLINE BITARTRATE\CYSTEINE\INOSITOL\MAMMAL LIVER:Diarrhoea:0.37), (CHONDROITIN:Diarrhoea:0.08), (CICLOPIROX OLAMINE:Diarrhoea:0.63), (CIFENLINE:Diarrhoea:0.9), (CLENBUTEROL HYDROCHLORIDE:Diarrhoea:1.06), (CLIKSTAR:Diarrhoea:0.48), (CLOFAZIMINE:Diarrhoea: 0.26), (COBICISTAT\ELVITEGRAVIR\EMTRICITABINE\TENOFOVIR ALAFENAMIDE FUMARATE:Diarrhoea:0.92), (CORTICOTROPIN:Diarrhoea:1.6), (CORTISONE ACETATE:Diarrhoea:0.52), (CROTALIDAE POLYVALENT IMMUNE FAB (OVINE):Diarrhoea:0.27), (CYANOCOBALAMIN\FOLIC ACID\PYRIDOXINE:Diarrhoea:0.6), (CYANOCOBALAMIN\LIDOCAINE HYDROCHLORIDE\PYRIDOXINE HYDROCHLORIDE\THIAMINE HYDROCHLORIDE:Diarrhoea:0.44), (CYANOCOBALAMIN\PYRIDOXINE HYDROCHLORIDE\THIAMINE DISULFIDE:Diarrhoea:0.4), (CYCLOPENTOLATE:Diarrhoea:0.16), (CYCLOPHOSPHAMIDE \CYTARABINE\DEXAMETHASONE\DOXORUBICIN\METHOTREXATE\VINCRISTINE: Diarrhoea:0.26), (CYPROTERONE:Diarrhoea:0.19), (DACLIZUMAB:Diarrhoea:2.65), (DANAZ OL:Diarrhoea:2.88), (DEFEROXAMINE MESYLATE:Diarrhoea:0.3), (DEGARELIX:Diarrhoea:1.1), (DEMECLOCYCLINE:Diarrhoea:0.22), (DENOPAMINE:Diarrhoea:0.07), (DEXMETHYLPHENIDATE HYDROCHLORIDE:Diarrhoea:0.15), (DEXTROAMPHETAMINE SULFATE:Diarrhoea:1.14), (DEXTROSE MONOHYDRATE\SODIUM CHLORIDE:Diarrhoea:0.26), (DIETARY SUPPLEMENT\HERBALS\MILK THISTLE:Diarrhoea:0.23), (DIFLUCORTOLONE VALERATE\LIDOCAINE:Diarrhoea:0.41), (DIFLUNISAL:Diarrhoea:0.72), (DILAZEP:Diarrhoea: 2.01), (DIMEMORFAN PHOSPHATE:Diarrhoea:0.79), (DIPYRONE\PROMETHAZINE HYDROCHLORIDE:Diarrhoea:0.64), (DISOPYRAMIDE PHOSPHATE:Diarrhoea:0.1), (DOXYCYCLINE HYDROCHLORIDE:Diarrhoea:0.06), (EDARAVONE:Diarrhoea:0.96), (ELBASVIR:Diarrhoea: 0.32), (ELBASVIR\GRAZOPREVIR:Diarrhoea:0.25), (ELTROMBOPAG OLAMINE:Diarrhoea:1.32), (EMPAGLIFLOZIN\LINAGLIPTIN:Diarrhoea:1.62), (EMTRICIT ABINE\RILPIVIRINE\TENOFOVIR DISOPROXIL FUMARATE:Diarrhoea:0.12), (EPACADOSTAT:Diarrhoea:0.03), (EPHEDRINE HYDROCHLORIDE:Diarrhoea:0.91), (EPHEDRINE HYDROCHLORIDE\GUAIFENESIN:Diarrhoea:0.08), (ESTRADIOL BENZOATE:Diarrhoea:0.03), (ESTROGENS\METHYLTESTOSTERONE:Diarrhoea:0), (ETHI NYL ESTRADIOL\ETHYNODIOL DIACETATE:Diarrhoea:0.01), (ETHIODIZED OIL:Diarrhoea:0.03), (ETOPOSIDE PHOSPHATE:Diarrhoea:0.82), (ETRETINATE:Diarrhoea:2.24), (FELBAMATE:Diarrhoea:0.47), (FENOPROFEN CALCIUM:Diarrhoea:0.14), (FERRIC HYDROXIDE:Diarrhoea:0.75), (FERRIC POLYSACCHARIDE COMPLEX:Diarrhoea:0.16), (FERROUS BISGLYCINATE:Diarrhoea:0.41), (FIBRINOGEN HUMAN:Diarrhoea:1.01), (FLAVIN ADENINE DINUCLEOTIDE:Diarrhoea:1.48), (FLOMOXEF:Diarrhoea:0.85), (FLOPROPIONE:Diarrhoea: 0.32), (FLUCYTOSINE:Diarrhoea:1.15), (FLUNARIZINE HYDROCHLORIDE:Diarrhoea:0.49), (FLUOROURACIL\IRINOTECAN\LEUCOVORIN:Diarrhoea: 0.63), (FLUOXETINE HYDROCHLORIDE\OLANZAPINE:Diarrhoea:0.04), (FLUPHENAZINE:Diarrhoea:0.08), (FO RMOTEROL FUMARATE\MOMETASONE FUROATE:Diarrhoea:1.74), (FROVATRIPTAN SUCCINATE:Diarrhoea:0.58), (FUROSEMIDE\SPIRONOLACTONE:Diarrhoea:0.18), (FURSU LTIAMINE:Diarrhoea:1.04), (GADOTERATE MEGLUMINE:Diarrhoea:0.14), (GARENOXACIN:Diarrhoea:0.32), (GENTIAN VIOLET:Diarrhoea:0.06), (GLUCONATE:Diarrhoea:0.17), (GLYCOPYRROLATE\INDACATE ROL MALEATE:Diarrhoea:1.18), (GRAZOPREVIR:Diarrhoea:0.28), (GUAIFENESIN\PHENYLEPH RINE HYDROCHLORIDE:Diarrhoea:0.26), (GUANABENZ ACETATE:Diarrhoea:1.1), (GUANFACINE HYDROCHLORIDE:Diarrhoea:0.9), (GUSPERIMUS TRIHYDROCHLORIDE:Diarrhoea:1.03), (HIGH DENSITY POLYETHYLENE:Diarrhoea:0.02), (HORSE CHESTNUT:Diarrhoea:0.11), (HYDROCHLOROTHIAZIDE\RAMIPRIL:Diarrhoea:0.76), (HY DROCORTISONE HEMISUCCINATE:Diarrhoea:0.21), (HYDROCORTISONE PROBUTATE:Diarrhoea:0.84), (HYDROCORTISONE SODIUM PHOSPHATE:Diarrhoea:0.34), (HYDROXOCOBALAMIN\PYRIDOXINE HYDROCHLORIDE\THIAMINE HYDROCHLORIDE:Diarrhoea:0.21), (IBRITUMOMAB TIUXETAN:Diarrhoea:1.65), (IFENPRODIL:Diarrhoea:0.67), (IGURATIMOD:Diarrhoea:1.07), (ILOPROST:Diarrhoea:1.61), (IMIDAPRIL HYDROCHLORIDE:Diarrhoea:0.48), (IMIGLUCERASE:Diarrhoea:1.5), (INDACATEROL MALEATE:Diarrhoea:0.08), (INFLIXIMAB-DYYB:Diarrhoea:0.89), (INSULIN ASPART\INSULIN DEGLUDEC:Diarrhoea:0.24), (INSULIN PORK\ANSULIN PURIFIED PORK:Diarrhoea:0.6), (INTERFERON ALFA-2A:Diarrhoea:1.95), (INTERFERON ALFA-2A OR INTERFERON ALFA-2B:Diarrhoea:0.67), (INTERFERON ALFA-N1:Diarrhoea:0.43), (INTERFERON BETA:Diarrhoea:0.3), (INTERFERON GAMMA-1B:Diarrhoea:2.06), (ISOFLURANE:Diarrhoea:0.97), (ISOLEUCINE\LEUCINE\VALINE:Diarrhoea: 0.2), (ISRADIPINE:Diarrhoea:0.15), (IVACAFTOR\LUMACAFTOR:Diarrhoea:0.3), (IXA BEPILONE:Diarrhoea:0.58), (KALLIDINOGENASE:Diarrhoea:0.48), (LACTITOL:Diarrhoea:0.23), (LANOLIN\MINERAL OIL\PETROLATUM:Diarrhoea:0.21), (LEVOFLOXACIN HYDROCHLORIDE:Diarrhoea:0.79), (LEVOSIMENDAN:Diarrhoea:0.32), (LIDOCAINE\TRI BENOSIDE:Diarrhoea:0.19), (LINAGLIPTIN\METFORMIN HYDROCHLORIDE:Diarrhoea:0.05), (LOPRAZOLAM:Diarrhoea:0.25), (LORCASERIN HYDROCHLORIDE:Diarrhoea:0.17), (LORNOXICAM:Diarrhoea:0.62), (LUMACAFTOR:Diarrhoea: 0.37), (LUTETIUM LU-177:Diarrhoea:0.84), (MAGNESIUM PIDOLATE:Diarrhoea:0.67), (MALTOSE:Diarrhoea:0.25), (MAPROTILINE HYDROCHLORIDE:Diarrhoea:0.23), (MEPOLIZUMAB:Diarrhoea:1.53), (METFORMIN HYDROCHLORIDE\SAXAGLIPTIN:Diarrhoea:0.93), (METHENAMINE:Diarrhoea:0.59), (ME THENAMINE MANDELATE:Diarrhoea:0.6), (METHENOLONE ACETATE:Diarrhoea:0.61), (METHENOLONE ENANTHATE:Diarrhoea:2.99), (METHYLENE BLUE:Diarrhoea:0.42), (METHYLNALTREXONE:Diarrhoea:0.59), (METRELEPTIN:Diarrhoea: 0.25), (MEXILETINE:Diarrhoea:0.89), (MITIGLINIDE CALCIUM HYDRATE\VOGLIBOSE:Diarrhoea:0.52), (MOGAMULIZUMAB:Diarrhoea:0.1), (NADIFLO XACIN:Diarrhoea:0.59), (NELARABINE:Diarrhoea:0.52), (NEUROTROPIN:Diarrhoea:0.37), (NILVADIPINE:Diarrhoea:0.22), (NIMODIPINE:Diarrhoea:0.32), (NUSINERSEN:Diarrhoea:0.48), (OBETICHOLIC ACID:Diarrhoea:0.2), (OK-432:Diarrhoea:0.46), (OLODATEROL HYDROCHLORIDE:Diarrhoea:0.22), (ORPHENADRINE:Diarrhoea:1.51), (OSPEMIFENE: Diarrhoea:0.99), (OXANDROLONE:Diarrhoea:0.29), (OXETHAZAINE:Diarrhoea:0.06), (OXYBUT YNIN\OXYBUTYNIN CHLORIDE:Diarrhoea:0.85), (PANTHENOL:Diarrhoea:0.33), (PAPAYA:Diarrhoea:0.03), (PAZ UFLOXACIN MESILATE:Diarrhoea:0.94), (PEGVISOMANT:Diarrhoea:0.35), (PEGZEREPOETIN ALFA:Diarrhoea:1.12), (PENTOSTATIN:Diarrhoea:1.81), (PERINDOPRIL ARGININE:Diarrhoea:0.36), (PHENTERMINE HYDROCHLORIDE\TOPIRAMATE:Diarrhoea:0.15), (PIMOBENDAN:Diarrhoea:1.84), (PIRA RUBICIN:Diarrhoea:0.23), (PIRARUBICIN HYDROCHLORIDE:Diarrhoea:0.3), (PIRBUTEROL ACETATE:Diarrhoea:0.37), (PIRENZEPINE:Diarrhoea:0.62), (PITOFENONE:Diarrhoea:0.09), (PIZOTYLINE:Diarrhoea:0.32), (PLASMA PROTEIN FRACTION (HUMAN):Diarrhoea:0.41), (POLYMYXIN:Diarrhoea:0.22), (POLYSTYRENE:Diarrhoea:0.8), (POLYSTYRENE SULFONIC ACID:Diarrhoea:1.16), (PORFIMER SODIUM:Diarrhoea:0.82), (POVIDONE:Diarrhoea:0.16), (PRAMLINTIDE ACETATE:Diarrhoea:0.14), (PRASUGREL:Diarrhoea:0.07), (PREGNENOLONE:Diarrhoea:0.11), (PROBUCOL:Diarrhoea:0.01), (PROCYCLIDINE:Diarrhoea:0.1), (PROTRIPTYLINE HYDROCHLORIDE:Diarrhoea:0.9), (PYRIMETHAMINE:Diarrhoea:0.71), (QUAZEPAM:Diarrhoea: 0.56), (QUERCUS SALICINA STEM:Diarrhoea:0.09), (RANIMUSTINE:Diarrhoea:0.69), (RHUBARB:Diarrhoea:1.03), (RILM ENIDINE PHOSPHATE:Diarrhoea:1.19), (RISEDRONIC ACID:Diarrhoea:0.44), (ROXATIDINE ACETATE HYDROCHLORIDE:Diarrhoea:0.56), (RUFINAMIDE:Diarrhoea:0.17), (RUPATADINE FUMARATE:Diarrhoea:0.54), (SALMETEROL XINAFOATE:Diarrhoea:1.36), (SAPROPTERIN DIHYDROCHLORIDE:Diarrhoea:0.32), (SARGRAMOSTIM:Diarrhoea:0.41), (SARPOGRELA TE HYDROCHLORIDE:Diarrhoea:1.05), (SELEGILINE HYDROCHLORIDE:Diarrhoea:0.45), (SENNOSIDE A:Diarrhoea:0.16), (SENNOSIDE B:Diarrhoea:0.76), (SENNOSIDES A AND B CALCIUM:Diarrhoea:0.12), (SERRAPEPTASE:Diarrhoea:0.64), (SIBUTRAMINE HYDROCHLORIDE:Diarrhoea:0.79), (SILICON DIOXIDE:Diarrhoea:0.5), (SIVELESTAT SODIUM:Diarrhoea:0.81), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Diarrhoea:0.55), (SOMATOSTATIN:Diarrhoea:0.57), (SORBITOL:Diarrhoea: 1.67), (SPIRAMYCIN:Diarrhoea:0.53), (STREPTOMYCIN SULFATE:Diarrhoea:0.02), (SULODEXIDE:Diarrhoea:0.24), (SULTOPRIDE HYDROCHLORIDE:Diarrhoea:0.29), (SUPLATAST TOSILATE:Diarrhoea:1.86), (TAMOXIFEN CITRATE:Diarrhoea:1.32), (TAURINE:Diarrhoea:0.19), (TAZOBACTAM SODIUM:Diarrhoea:0.06), (TECELEUKIN:Diarrhoea:0.32), (TEGASEROD MALEATE:Diarrhoea:0.12), (TEMOCAPRIL:Diarrhoea:0.24), (TEMOCAPRIL HYDROCHLORIDE:Diarrhoea:1.6), (TESTOSTERONE UNDECANOATE:Diarrhoea:1.68), (TETRABENAZINE:Diarrhoea:0.11), (THIAMYLAL SODIUM:Diarrhoea:0.94), (THIOTHIXENE:Diarrhoea:0.07), (THROMBOPOIETIN:Diarrhoea: 0.63), (TIANEPTINE:Diarrhoea:0.47), (TICLOPIDINE:Diarrhoea:1.12), (TILIDINE:Diarrhoea: 0.59), (TIMOLOL MALEATE\TRAVOPROST:Diarrhoea:0.82), (TIMOLOL\TRAVOPROST:Diarrhoea:0.13), (TIS AGENLECLEUCEL:Diarrhoea:0.23), (TOPIROXOSTAT:Diarrhoea:0.29), (TRIMIPRAMINE MALEATE:Diarrhoea:1.58), (TRIPTORELIN PAMOATE:Diarrhoea:0.93), (TULOBUTEROL HYDROCHLORIDE:Diarrhoea:0.06), (VELAGLUCERASE ALFA:Diarrhoea:0.16), (VINBLASTINE SULFATE:Diarrhoea:0.17), (VINDESINE SULFATE:Diarrhoea:0.15), (VINPOCETINE:Diarrhoea:0.06), (YEAST:Diarrhoea:1.13), (ZICON OTIDE ACETATE:Diarrhoea:1.14), (ZILEUTON:Diarrhoea:1.24), (SELEXIPAG:Diastolic dysfunction:0.04), (ABACAVIR SULFATE\DOLUTEGRAVIR SODIUM\LAMIVUDINE:Diffuse large B-cell lymphoma:0.18), (ACETAMINOPHEN\PROPOXYPHENE NAPSYLATE:Diffuse large B-cell lymphoma:0.34), (ADO-TRASTUZUMAB EMTANSINE:Diffuse large B-cell lymphoma:0.5), (AGALSIDASE BETA:Diffuse large B-cell lymphoma:0.25), (ALBUTEROL\IPRATROPIUM:Diffuse large B-cell lymphoma:0.2), (ALCOHOL:Diffuse large B-cell lymphoma:0.06), (ALUMINUM HYDROXIDE\MAGNESIUM OXIDE\OXETHAZAINE:Diffuse large B-cell lymphoma:0.29), (AMANTADINE:Diffuse large B-cell lymphoma:0.23), (AMLODIPINE BESYLATE\HYDROCHLOROTHIAZIDE\VALSARTAN:Diffuse large B-cell lymphoma:0.1), (AMLODIPINE\VALSARTAN:Diffuse large B-cell lymphoma:0.45), (AMPHETAMINE ASPARTATE\AMPHETAMINE SULFATE\DEXTROAMPHETAMINE SACCHARATE\DEXTROAMPHETAMINE SULFATE:Diffuse large B-cell lymphoma:0.5), (ANIDULAFUNGIN:Diffuse large B-cell lymphoma:0.24),(ARMODAFINIL:Diffuse large B-cell lymphoma:0.36), (ASPARAGINASE:Diffuse large B-cell lymphoma:0.03), (ASPIRIN\MAGNESIUM OXIDE:Diffuse large B-cell lymphoma:0.17), (AXITINIB:Diffuse large B-cell lymphoma:0.05), (AZILSARTAN KAMEDOXOMIL:Diffuse large B-cell lymphoma:0.11), (BELATACEPT:Diffuse large B-cell lymphoma:0.1), (BENAZEPRIL\BENAZEPRIL HYDROCHLORIDE:Diffuse large B-cell lymphoma:0.54), (BILASTINE:Diffuse large B-cell lymphoma:0.03), (BRINZOLAMIDE:Diffuse large B-cell lymphoma:0.43), (BUDESONIDE\FORMOTEROL FUMARATE:Diffuse large B-cell lymphoma:0.49), (BUPRENORPHINE HYDROCHLORIDE\NALOXONE HYDROCHLORIDE:Diffuse large B-cell lymphoma:0.02), (CABAZITAXEL:Diffuse large B-cell lymphoma:0.04), (CALCIUM CITRATE:Diffuse large B-cell lymphoma:0.67), (CALCIUM\MAGNESIUM:Diffuse large B-cell lymphoma:0.09), (CANAGLIFLOZIN\METFORMIN HYDROCHLORIDE:Diffuse large B-cell lymphoma:0.25), (CANAKINUMAB:Diffuse large B-cell lymphoma:0.33), (CARVEDILOL PHOSPHATE:Diffuse large B-cell lymphoma:0.69), (CASANTHRANOL\DOCUSATE SODIUM:Diffuse large B-cell lymphoma:0.09), (CERITINIB:Diffuse large B-cell lymphoma:0.19), (CERTOLIZUMAB PEGOL:Diffuse large B-cell lymphoma:1.24), (CETUXIMAB:Diffuse large B-cell lymphoma:0.35), (CLINDAMYCIN HYDROCHLORIDE:Diffuse large B-cell lymphoma:0.16), (COLISTIMETHATE SODIUM:Diffuse large B-cell lymphoma:0.11), (COLISTIN:Diffuse large B-cell lymphoma:0.21), (CYAMEMAZINE:Diffuse large B-cell lymphoma:0.02), (DABIGATRAN:Diffuse large B-cell lymphoma:0.28), (DABRAFENIB:Diffuse large B-cell lymphoma:0.55), (DACLIZUMAB:Diffuse large B-cell lymphoma:0.7), (DANAZOL:Diffuse large B-cell lymphoma:0.8), (DAPAGLIFLOZIN PROPANEDIOL:Diffuse large B-cell lymphoma:0.15), (DASATINIB:Diffuse large B-cell lymphoma:1.9), (DAUNORUBICIN HYDROCHLORIDE:Diffuse large B-cell lymphoma:0.02), (DEVICE:Diffuse large B-cell lymphoma:0.53), (DEXAMETHASONE ACETATE:Diffuse large B-cell lymphoma:0.99), (DIPYRONE:Diffuse large B-cell lymphoma:0.65), (DOCETAXEL:Diffuse large B-cell lymphoma:0.13), (DOXEPIN HYDROCHLORIDE:Diffuse large B-cell lymphoma:0.09), (DRONEDARONE:Diffuse large B-cell lymphoma:0.07), (EDOXABAN TOSYLATE:Diffuse large B-cell lymphoma:0.71), (ELETRIPTAN HYDROBROMIDE:Diffuse large B-cell lymphoma:0.58), (ELOTUZUMAB:Diffuse large B-cell lymphoma:0.96), (EMPAGLIFLOZIN\LINAGLIPTIN:Diffuse large B-cell lymphoma:0.58), (EPLERENONE:Diffuse large B-cell lymphoma:0.71), (ERTAPENEM SODIUM:Diffuse large B-cell lymphoma:0.08), (ETORICOXIB:Diffuse large B-cell lymphoma:0.61), (EVOLOCUMAB:Diffuse large B-cell lymphoma:0.2), (FENOTEROL HYDROBROMIDE:Diffuse large B-cell lymphoma:0.19), (FERROUS GLUCONATE:Diffuse large B-cell lymphoma:0.15), (FERROUS SULFATE\FOLIC ACID:Diffuse large B-cell lymphoma:0.34), (FERROUS SULFATE\GLYCINE:Diffuse large B-cell lymphoma:0.13), (FESOTERODINE FUMARATE:Diffuse large B-cell lymphoma:1.03), (FLUOCINONIDE:Diffuse large B-cell lymphoma:0.32), (FLUTICASONE FUROATE:Diffuse large B-cell lymphoma:0.52), (FORMOTEROL FUMARATE:Diffuse large B-cell lymphoma:0.8), (FOSFOMYCIN:Diffuse large B-cell lymphoma:0.33), (GEMFIBROZIL:Diffuse large B-cell lymphoma:0.43), (GIMERACIL\OTERACIL\TEGAFUR:Diffuse large B-cell lymphoma:0.34), (GLYCOPYRRONIUM:Diffuse large B-cell lymphoma:0.01), (GLYCOPYRRONIUM\INDACATEROL:Diffuse large B-cell lymphoma:0.91), (HYOSCYAMINE:Diffuse large B-cell lymphoma:0.15), (IMATINIB MESYLATE:Diffuse large B-cell lymphoma:1.39), (IMIPRAMINE HYDROCHLORIDE:Diffuse large B-cell lymphoma:0.21), (INTERFERON:Diffuse large B-cell lymphoma:0.21), (INTERFERON ALFA-2A:Diffuse large B-cell lymphoma:0.38), (INTERFERON GAMMA-1B:Diffuse large B-cell lymphoma:0.33), (IRINOTECAN HYDROCHLORIDE:Diffuse large B-cell lymphoma:0.3), (LEVOTHYROXINE SODIUM\POTASSIUM IODIDE:Diffuse large B-cell lymphoma:0.16), (LITHIUM CARBONATE:Diffuse large B-cell lymphoma:1.09), (MAGNESIUM ASPARTATE\POTASSIUM ASPARTATE:Diffuse large B-cell lymphoma:0.1), (MECLIZINE\MECLIZINE HYDROCHLORIDE:Diffuse large B-cell lymphoma:0.26), (MERCAPTOPURINE:Diffuse large B-cell lymphoma:0.52), (METAXALONE:Diffuse large B-cell lymphoma:0.9), (METHENAMINE HIPPURATE:Diffuse large B-cell lymphoma:0.93), (METHENOLONE ENANTHATE:Diffuse large B-cell lymphoma:0.21), (MINOCYCLINE\MINOCYCLINE HYDROCHLORIDE:Diffuse large B-cell lymphoma:1.17), (MODAFINIL:Diffuse large B-cell lymphoma:0.1), (MORPHINE SULFATE\NALTREXONE HYDROCHLORIDE:Diffuse large B-cell lymphoma:0.02), (NABUMETONE:Diffuse large B-cell lymphoma:0.23), (NELARABINE:Diffuse large B-cell lymphoma:0.07), (NILOTINIB:Diffuse large B-cell lymphoma:2.18), (NINTEDANIB:Diffuse large B-cell lymphoma:0.52), (NITROFURANTOIN\NITROFURANTOIN MONOHYDRATE:Diffuse large B-cell lymphoma:0.14), (OLAPARIB:Diffuse large B-cell lymphoma:0), (OLODATEROL HYDROCHLORIDE\TIOTROPIUM BROMIDE MONOHYDRATE:Diffuse large B-cell lymphoma:0.41), (PAZOPANIB HYDROCHLORIDE:Diffuse large B-cell lymphoma:0.99), (PEGASPARGASE:Diffuse large B-cell lymphoma:0.33), (PEMETREXED:Diffuse large B-cell lymphoma:1.02), (PHENOBARBITAL:Diffuse large B-cell lymphoma:1.42), (PHENYTOIN SODIUM:Diffuse large B-cell lymphoma:0.85), (PHLOROGLUCINOL\1,3,5-TRIMETHOXYBENZENE:Diffuse large B-cell lymphoma:0.19), (PIROXICAM:Diffuse large B-cell lymphoma:0.27), (PITAVASTATIN CALCIUM:Diffuse large B-cell lymphoma:0.88), (PNEUMOCOCCAL VACCINE, POLYVALENT 23:Diffuse large B-cell lymphoma:0.1), (PONATINIB:Diffuse large B-cell lymphoma:1.67), (PRAMIPEXOLE DIHYDROCHLORIDE:Diffuse large B-cell lymphoma:0.65), (PYRIDOSTIGMINE BROMIDE:Diffuse large B-cell lymphoma:0.28), (QUINAPRIL:Diffuse large B-cell lymphoma:0.01), (RALOXIFENE HYDROCHLORIDE:Diffuse large B-cell lymphoma:0.28), (RAMUCIRUMAB:Diffuse large B-cell lymphoma:0.17), (RETINOL:Diffuse large B-cell lymphoma:0.28), (RIVASTIGMINE TARTRATE:Diffuse large B-cell lymphoma:0.21), (ROFLUMILAST:Diffuse large B-cell lymphoma:0.73), (ROPINIROLE HYDROCHLORIDE:Diffuse large B-cell lymphoma:0.51), (SACUBITRIL\VALSARTAN:Diffuse large B-cell lymphoma:0.41), (SECUKINUMAB:Diffuse large B-cell lymphoma:0.26), (SODIUM CITRATE:Diffuse large B-cell lymphoma:0.1), (SODIUM OXYBATE:Diffuse large B-cell lymphoma:0.3), (SUNITINIB MALATE:Diffuse large B-cell lymphoma:0.46), (TERBUTALINE:Diffuse large B-cell lymphoma:0.41), (TETRACYCLINE:Diffuse large B-cell lymphoma:0.76), (TIOTROPIUM:Diffuse large B-cell lymphoma:0.88), (TOBRAMYCIN:Diffuse large B-cell lymphoma:0.27), (TOCOPHEROL:Diffuse large B-cell lymphoma:0.14), (TRAMETINIB:Diffuse large B-cell lymphoma:0.46), (TRIAMTERENE:Diffuse large B-cell lymphoma:0.25), (UMECLIDINIUM BROMIDE\VILANTEROL TRIFENATATE:Diffuse large B-cell lymphoma:0.12), (VEMURAFENIB:Diffuse large B-cell lymphoma:0.16), (ZINC:Diffuse large B-cell lymphoma:0.45), (BENAZEPRIL HYDROCHLORIDE:Discomfort:0.02), (BUMETANIDE:Discomfort:0.88), (BUPRENORPHIN E HYDROCHLORIDE\NALOXONE HYDROCHLORIDE:Discomfort:0.19), (CANDESARTAN CILEXETIL\HYDROCHLOROTHIAZIDE:Discomfort:0.08), (CLINDAMYCIN\CLINDAMYC IN PHOSPHATE:Discomfort:0.8), (CLOTRIMAZOLE:Discomfort:0.05), (DASATINIB:Discomfort:0.12), (DILTIAZEM HYDROCHLORIDE:Discomfort:0.95), (DUTASTERIDE:Discomfort:0.17), (ENOXAPARIN: Discomfort:0.88), (ESZOPICLONE:Discomfort:0.15), (ETHAMBUTOL HYDROCHLORIDE:Discomfort:0.92), (ETORICOXIB:Discomfort:0.06), (FLECAINIDE:Discomfort: 0.16), (IBRUTINIB:Discomfort:1.08), (INSULIN ASPART:Discomfort:0.45), (IRON:Discomfort:0.72), (METAXALONE:Discomfort:0), (NYSTA TIN:Discomfort:0.13), (OLANZAPINE:Discomfort:0.1), (PANTOPRAZOLE MAGNESIUM:Discomfort:0.11), (PAROXETINE\PAROXETINE HYDROCHLORIDE:Discomfort:0.08), (PENICILLIN:Discomfort:0), (PHENYTOIN:Discomfort: 0.04), (POLYETHYLENE GLYCOLS:Discomfort:0.45), (PRAMIPEXOLE DIHYDROCHLORIDE:Discomfort:0.12), (PRIMIDONE:Discomfort:0.24), (RABEPRAZOLE SODIUM:Discomfort:0.43), (SACUBITRIL\VALSARTAN:Discomfort:0.4), (SOTALOL:Discomfort: 0.28), (TERIFLUNOMIDE:Discomfort:0.21), (TIZANIDINE HYDROCHLORIDE:Discomfort:0.12), (TOPIRAMATE:Discomfort:0.86), (TRAZODONE HYDROCHLORIDE:Discomfort:0.87), (TRIAMCINOLONE ACETONIDE:Discomfort:0.2), (DASATINIB:Disease progression:0.08), (DAUNORUBICIN:Disease progression:0.15), (IBRITUMOMAB TIUXETAN:Disease progression:0.21), (NILOTINIB:Disease progression:0.2), (PAZOPANIB HYDROCHLORIDE:Disease progression:0.33), (REGORAFENIB:Disease progression:0.04), (BENDAMUSTINE HYDROCHLORIDE:Disease recurrence:0.08), (LEVOLEUCOVORIN CALCIUM:Disease recurrence:0.04), (PYRAZINAMIDE:Disseminated Bacillus Calmette-Guerin infection:0.54), (ATAZANAVIR:Disseminated tuberculosis:0.46), (CALCIUM CARBONATE\CHOLECALCIFEROL:Disturbance in attention:0.1), (CEPHALEXIN:Disturbance in attention:0.06), (CERTOLIZUMAB PEGOL:Disturbance in attention:0.27), (DEXLANSOPRAZOLE:Disturbance in attention:0.21), (DILTIAZEM HYDROCHLORIDE:Disturbance in attention:0.02), (DOCUSATE SODIUM:Disturbance in attention:0.6), (ELETRIPTAN HYDROBROMIDE:Disturbance in attention:0.26), (EVOLOCUMAB:Disturbance in attention:0.4), (FERROUS SULFATE\FERROUS SULFATE, DRIED:Disturbance in attention:0.12), (INDOMETHACIN:Disturbance in attention:0.24), (METHYLPREDNISOLONE SODIUM SUCCINATE:Disturbance in attention:0.22), (METRONIDAZOLE:Disturbance in attention:0.25), (PALIPERIDONE PALMITATE:Disturbance in attention:0.54), (PANTOPRAZOLE:Disturbance in attention:0.36), (SACUBITRIL\VALSARTAN:Disturbance in attention:0.2), (TAMSULOSIN:Disturbance in attention:0.13), (TRIAMCINOLONE ACETONIDE:Disturbance in attention:0.14), (VARENICLINE TARTRATE:Disturbance in attention:1.64), (ZONISAMIDE:Disturbance in attention:0.32), (ABOBOTULINUMTOXINA:Diuretic therapy:0.11), (ACETAMINOPHEN\CAFFEINE\OPIUM:Diuretic therapy:0.49), (ACETAMINOPHEN\METHOCARBAMOL:Diuretic therapy:0.12), (ACTIVATED CHARCOAL:Diuretic therapy:0.75), (ADO-TRASTUZUMAB EMTANSINE:Diuretic therapy:1.58), (ALUMINUM HYDROXIDE\MAGNESIUM OXIDE\OXETHAZAINE:Diuretic therapy:0.03), (AMINO ACIDS\DEXTROSE\ELECTROLYTES NOS:Diuretic therapy:0.5), (AMISULPRIDE:Diuretic therapy:1), (AMLODIPINE BESYLATE\TELMISARTAN:Diuretic therapy:0.12), (AMLODIPINE\PERINDOPRIL:Diuretic therapy:0.66), (AMMONIUM CHLORIDE\DEXTROMETHORPHAN\SODIUM CITRATE:Diuretic therapy:0.28), (AMOXICILLIN\CLARITHROMYCIN\ESOMEPRAZOLE:Diuretic therapy:0.1), (ANAKINRA:Diuretic therapy:0.27), (ANHYDROUS CITRIC ACID\ASPIRIN\SODIUM BICARBONATE:Diuretic therapy:0.14), (ANIDULAFUNGIN:Diuretic therapy:0.12), (ARGININE:Diuretic therapy:0.02), (ARIPIPRAZOLE LAUROXIL:Diuretic therapy:0.06), (ASCORBIC ACID\BIOTIN\CYANOCOBALAMIN\FOLIC ACID\NIACIN\PANTOTHENIC ACID\PYRIDOXINE\RIBOFLAVIN\THIAMINE:Diuretic therapy:0.08), (ASUNAPREVIR:Diuretic therapy:0.43), (ATORVASTATIN CALCIUM\EZETIMIBE:Diuretic therapy:0.03), (ATOVAQUONE:Diuretic therapy:1.23), (ATRACURIUM:Diuretic therapy:0.1), (ATROPINE\DIPHENOXYLATE:Diuretic therapy:0.51), (BAMIFYLLINE HYDROCHLORIDE:Diuretic therapy:0.26), (BECLOMETHASONE\FORMOTEROL:Diuretic therapy:0.38), (BELATACEPT:Diuretic therapy:0.94), (BENZTROPINE:Diuretic therapy:0.17), (BENZTROPINE MESYLATE:Diuretic therapy:0.24), (BETAMETHASONE\CALCIPOTRIENE:Diuretic therapy:0.72), (BETAXOLOL HYDROCHLORIDE:Diuretic therapy:0.13), (BETHANECHOL CHLORIDE:Diuretic therapy:0.6), (BICARBONATE ION:Diuretic therapy:0.27), (BIFIDOBACTERIUM ANIMALIS SUBSP. LACTIS:Diuretic therapy:0.11), (BILASTINE:Diuretic therapy:0.32), (BLESELUMAB:Diuretic therapy:0.04), (BLINATUMOMAB:Diuretic therapy:1.56), (BOCEPREVIR:Diuretic therapy:0.2), (BOCOCIZUMAB:Diuretic therapy:0.34), (BOSUTINIB:Diuretic therapy:0.04), (BUPIVACAINE HYDROCHLORIDE:Diuretic therapy:0.14), (CABAZITAXEL:Diuretic therapy:0.58), (CABOZANTINIB S-MALATE:Diuretic therapy:0.19), (CALCIUM CHLORIDE\DEXTROSE MONOHYDRATE\MAGNESIUM CHLORIDE\SODIUM CHLORIDE\SODIUM LACTATE:Diuretic therapy:0.08), (CALCIUM PANTOTHENATE\CYANOCOBALAMIN\NIACINAMIDE\PYRIDOXINE HYDROCHLORIDE\RIBOFLAVIN\THIAMINE MONONITRATE:Diuretic therapy:0.12), (CALCIUM\MAGNESIUM\ZINC:Diuretic therapy:1.13), (CARBOMER:Diuretic therapy:0.08), (CEFOTAXIME\CEFOTAXIME SODIUM:Diuretic therapy:0.33), (CERIVASTATIN SODIUM:Diuretic therapy:0.1), (CERTOPARIN SODIUM:Diuretic therapy:0.05), (CETUXIMAB:Diuretic therapy:0.35), (CHLORDIAZEPOXIDE HYDROCHLORIDE\CLIDINIUM BROMIDE:Diuretic therapy:0.84), (CHOLESTEROL:Diuretic therapy:0.03), (CICLOPIROX:Diuretic therapy:0.11), (CISATRACURIUM:Diuretic therapy:0.16), (CLADRIBINE:Diuretic therapy:0.04), (CLOMIPRAMINE HYDROCHLORIDE:Diuretic therapy:0.48), (CORTISONE ACETATE:Diuretic therapy:0.3), (CYAMEMAZINE:Diuretic therapy:0.12), (CYPROHEPTADINE HYDROCHLORIDE:Diuretic therapy:0.06), (DACLIZUMAB:Diuretic therapy:1.9), (DANTROLENE SODIUM:Diuretic therapy:0.25), (DEFERIPRONE:Diuretic therapy:0.26), (DEGARELIX:Diuretic therapy:0.15), (DEGARELIX ACETATE:Diuretic therapy:0.02), (DESFLURANE:Diuretic therapy:0.8), (DEXTROAMPHETAMINE:Diuretic therapy:0), (DEXTROAMPHETAMINE SULFATE:Diuretic therapy:1.11), (DOLASETRON MESYLATE:Diuretic therapy:0.63), (DOXYCYCLINE HYCLATE:Diuretic therapy:0.8), (DROXIDOPA:Diuretic therapy:0.28), (ELOTUZUMAB:Diuretic therapy:1.28), (ELTROMBOPAG OLAMINE:Diuretic therapy:0.38), (EMPAGLIFLOZIN\LINAGLIPTIN:Diuretic therapy:1.37), (ENALAPRIL MALEATE\LERCANIDIPINE HYDROCHLORIDE:Diuretic therapy:0.15), (ERIBULIN:Diuretic therapy:0.43), (ESMOLOL HYDROCHLORIDE:Diuretic therapy:0.7), (ETHINYL ESTRADIOL\NORGESTREL:Diuretic therapy:0.12), (ETOPOSIDE PHOSPHATE:Diuretic therapy:0.37), (FERRIC HYDROXIDE:Diuretic therapy:0.2), (FERROUS SULFATE ANHYDROUS:Diuretic therapy:0.46), (FERROUS SULFATE\GLYCINE:Diuretic therapy:0.65), (FLURBIPROFEN:Diuretic therapy:0.02), (FONDAPARINUX SODIUM:Diuretic therapy:0.9), (FORMOTEROL FUMARATE\MOMETASONE FUROATE:Diuretic therapy:1.12), (FOSFOMYCIN:Diuretic therapy:0.58), (GIMERACIL\OTERACIL\TEGAFUR:Diuretic therapy:0.48), (GLIMEPIRIDE\ROSIGLITAZONE MALEATE:Diuretic therapy:0.48), (GLUTAMINE:Diuretic therapy:0.02), (GLYCOPYRROLATE\INDACATEROL MALEATE:Diuretic therapy:0.7), (GUSPERIMUS TRIHYDROCHLORIDE:Diuretic therapy:0.3), (HYALURONATE SODIUM:Diuretic therapy:0.4), (HYDROCHLOROTHIAZIDE\RAMIPRIL:Diuretic therapy:0.29), (HYDROCODONE BITARTRATE\IBUPROFEN:Diuretic therapy:0.66), (IBRITUMOMAB TIUXETAN:Diuretic therapy:0.89), (IMMUNE GLOBULIN NOS:Diuretic therapy:0.05), (INFLIXIMAB-DYYB:Diuretic therapy:0.11), (INTERFERON:Diuretic therapy:1.31), (INTERFERON ALFA-2A:Diuretic therapy:1.44), (INTERFERON GAMMA-1B:Diuretic therapy:0.09), (IOPAMIDOL:Diuretic therapy:0.08), (IRINOTECAN:Diuretic therapy:0.32), (LACTASE:Diuretic therapy:1), (LANREOTIDE:Diuretic therapy:0.03), (LAPATINIB DITOSYLATE:Diuretic therapy:0.75), (LENVATINIB:Diuretic therapy:0.33), (LEVODOPA:Diuretic therapy:0.05), (LEVOFLOXACIN HYDROCHLORIDE:Diuretic therapy:0.42), (LEVOLEUCOVORIN CALCIUM:Diuretic therapy:0.1), (LEVOMEFOLATE CALCIUM:Diuretic therapy:0.43), (LEVOTHYROXINE SODIUM\POTASSIUM IODIDE:Diuretic therapy:0.17), (LINSIDOMINE:Diuretic therapy:0.17), (LUTETIUM LU-177: Diuretic therapy:0.18), (MAGNESIUM ASPARTATE HYDROCHLORIDE:Diuretic therapy:0.33), (MAGNESIUM ASPARTATE\POTASSIUM ASPARTATE:Diuretic therapy:0.2), (MEDROXYPROGESTERONE:Diuretic therapy:0.33), (MEPERIDINE HYDROCHLORIDE:Diuretic therapy:1.52), (MEPOLIZUMAB:Diuretic therapy:0.83), (METFORMIN HYDROCHLORIDE\SAXAGLIPTIN:Diuretic therapy:0.14), (METFORMIN\SITAGLIPTIN:Diuretic therapy:0), (METHENOLONE ENANTHATE:Diuretic therapy:0.44), (METILDIGOXIN:Diuretic therapy:0.06), (METRELEPTIN:Diuretic therapy:0.1), (MIDODRINE HYDROCHLORIDE:Diuretic therapy:0.84), (MILRINONE LACTATE:Diuretic therapy:0.09), (NALOXONE:Diuretic therapy:0.29), (NALOXONE HYDROCHLORIDE\TILIDINE HYDROCHLORIDE:Diuretic therapy:1.51), (NARATRIPTAN HYDROCHLORIDE:Diuretic therapy:0.29), (OLOPATADINE:Diuretic therapy:0.48), (ORPHENADRINE:Diuretic therapy:0.54), (PEGINTERFERON BETA-1A:Diuretic therapy:1.35), (PENTOSTATIN:Diuretic therapy:0.58), (PERTUZUMAB:Diuretic therapy:0.7), (PIMECROLIMUS:Diuretic therapy:0.32), (PIROXICAM:Diuretic therapy:1.28), (POLYETHYLENE GLYCOL 3350\POTASSIUM CHLORIDE\SODIUM BICARBONATE\SODIUM CHLORIDE\SODIUM SULFATE ANHYDROUS:Diuretic therapy:0.6), (POTASSIUM PHOSPHATE, MONOBASIC:Diuretic therapy:0.03), (PRISTINAMYCIN:Diuretic therapy:0.26), (PROGESTERONE:Diuretic therapy:1.13), (PROTRIPTYLINE HYDROCHLORIDE:Diuretic therapy:0.3), (PYRIDOSTIGMINE BROMIDE:Diuretic therapy:0.9), (QUINACRINE:Diuretic therapy:0.17), (RAMUCIRUMAB:Diuretic therapy:0.25), (REGORAFENIB:Diuretic therapy:0.77), (RETINOL:Diuretic therapy:1.1), (RILMENIDINE PHOSPHATE:Diuretic therapy:0.68), (SELEGILINE:Diuretic therapy:0.14), (SODIUM CITRATE:Diuretic therapy:0.32), (SORBITOL:Diuretic therapy:0.05), (SULBACTAM SODIUM:Diuretic therapy:0.16), (TEDUGLUTIDE\WATER:Diuretic therapy:0.22), (TELOTRISTAT ETHYL:Diuretic therapy:0.2), (TEMSIROLIMUS:Diuretic therapy:0.14), (TERBUTALINE:Diuretic therapy:0.88), (TESTOSTERONE ENANTHATE:Diuretic therapy:0.07), (TESTOSTERONE UNDECANOATE:Diuretic therapy:1.27), (TETRACYCLINE:Diuretic therapy:1.83), (THIOCTIC ACID:Diuretic therapy:0.5), (THIOPENTAL:Diuretic therapy:0.14), (TIMOLOL MALEATE\TRAVOPROST:Diuretic therapy:0.12), (TIPIRACIL HYDROCHLORIDE\TRIFLURIDINE:Diuretic therapy:0.29), (TRAMETINIB DIMETHYL SULFOXIDE:Diuretic therapy:0.4), (TRIMIPRAMINE MALEATE:Diuretic therapy:0.46), (TROSPIUM CHLORIDE:Diuretic therapy:1.17), (UBIQUINOL:Diuretic therapy:0.03), (UMECLIDINIUM BROMIDE:Diuretic therapy:0.99), (VEMURAFENIB:Diuretic therapy:0.66), (VINORELBINE\VINORELBINE TARTRATE:Diuretic therapy:0.25), (VISMODEGIB:Diuretic therapy:0.09), (WARFARIN POTASSIUM:Diuretic therapy:0.22), (ZILEUTON:Diuretic therapy:0.63), (AMITRIPTYLINE HYDROCHLORIDE:Diverticulitis:0.24), (AZITHROMYCIN DIHYDRATE:Diverticulitis:0.14), (BORTEZOMIB:Diverticulitis:0.29), (BRINZOLAMIDE: Diverticulitis:0.17), (BUDESONIDE\FORMOTEROL FUMARATE:Diverticulitis:0.24), (BUMETANIDE:Diverticulitis:0.92), (CARFILZOMIB:Diverticulitis: 0.34), (CARISOPRODOL:Diverticulitis:0.34), (COLESEVELAM HYDROCHLORIDE:Diverticulitis:0.12), (DACLIZUMAB:Diverticulitis:0.04), (DASATINIB: Diverticulitis:0.96), (DESLORATADINE:Diverticulitis:0.24), (DOXAZOSIN\DOXAZOSIN MESYLATE:Diverticulitis:0.32), (DOXEPIN HYDROCHLORIDE:Diverticulitis:0.08), (DUTASTERIDE:Diverticulitis:0.14), (EVEROLIMUS:Diverticulitis:0.53), (FESOTERODINE FUMARATE:Diverticulitis:0.26), (FLECAINIDE:Diverticulitis:0.51), (FORMOTEROL FUMARATE:Diverticulitis:0.05), (GLICLAZIDE:Diverticulitis:0.03), (HERBALS\TURMERIC: Diverticulitis:0.17), (HYDROCHLOROTHIAZIDE\IRBESARTAN:Diverticulitis:0.1), (HYDRO XYCHLOROQUINE DIPHOSPHATE:Diverticulitis:0.15), (HYDROXYZINE PAMOATE:Diverticulitis:0.65), (ILOPROST:Diverticulitis:0.16), (IMIPRAMINE HYDROCHLORIDE:Diverticulitis:0.14), (INDOMETHACIN:Diverticulitis:0.43), (INSULIN HUMAN:Diverticulitis:0.38), (IPRATROPIUM BROMIDE:Diverticulitis:0.03), (LABETALOL\LABETALOL HYDROCHLORIDE:Diverticulitis:0.42), (LERCANIDIPINE:Diverticulitis:0.32), (LINAGLIPTI N:Diverticulitis:0.22), (LISDEXAMFETAMINE DIMESYLATE:Diverticulitis:0.09), (LOMITAPIDE MESYLATE:Diverticulitis:0.04), (MEGESTROL ACETATE:Diverticulitis:0.04), (METHENAMINE HIPPURATE:Diverticulitis:0.19), (METHYLPHENIDATE HYDROCHLORIDE:Diverticulitis:0.03), (METHYLPREDNISOLONE ACETATE:Diverticulitis:0.45), (MODAFINIL:Diverticulitis:0.44), (NALOXONE HYDROCHLORIDE\OXYCODONE HYDROCHLORIDE:Diverticulitis:0.32), (NEBIVOLOL:Diverticulitis:0.94), (NIACIN:Diverticulitis: 0.34), (OMEGA-3-ACID ETHYL ESTERS:Diverticulitis:0.61), (OXCARBAZEPINE:Diverticulitis:0.96), (PANTOPRAZOLE MAGNESIUM:Diverticulitis:0.73), (PAROXETINE:Diverticulitis:0.56), (PAZOPANIB HYDROCHLORIDE:Diverticulitis:0.17), (PEGFILGRASTIM:Diverticulitis:0.01), (PENICILLI N:Diverticulitis:0.38), (PERINDOPRIL:Diverticulitis:0.28), (PONATINIB:Diverticulitis:0.2), (PR AVASTATIN SODIUM:Diverticulitis:0.92), (PYRIDOXINE:Diverticulitis:0.12), (QUETIAPINE:Diverticulitis: 0.03), (RABEPRAZOLE:Diverticulitis:0.43), (SACUBITRIL\VALSARTAN:Diverticulitis:0.97), (SULFAMETHOXAZOLE:Diverticulitis:0.15), (SUMATRIPTAN:Diverticulitis:0.03), (SUMAT RIPTAN\SUMATRIPTAN SUCCINATE:Diverticulitis:0.52), (TERIFLUNOMIDE:Diverticulitis:0.54), (TETRACYCLINE: Diverticulitis:0.37), (THALIDOMIDE:Diverticulitis:0.16), (TIOTROPIUM:Diverticulitis:0.02), (TOCILIZUMAB:Diverticulitis:0.11), (VILAZODONE HYDROCHLORIDE:Diverticulitis:0.04), (CERTOLIZUMAB PEGOL:Diverticulum:0.21), (DIGOXIN:Diverticulum:0.37), (ESCITALOPRAM OXALATE:Diverticulum:0.09), (IBRUTINIB:Diverticulum:0.33), (LOVASTATIN:Diverticulum: 0.22), (SACUBITRIL\VALSARTAN:Diverticulum:0.13), (VERAPAMIL HYDROCHLORIDE:Diverticulum:0.02), (.ALPHA.-TOCOPHEROL ACETATE, DL-\ASCORBIC ACID\CYANOCOBALAMIN\FLUORIDE ION\FOLIC ACID\NIACIN\PYRIDOXINE\RIBOFLAVIN\THIAMINE\VITAMIN A\VITAMIN D:Dizziness:0.27), (.ALPHA.1-PROTEINASE INHIBITOR HUMAN:Dizziness:0.39), (ACENOCOUMAROL:Dizziness:0.74), (ACETAMINOPHEN\OXYC ODONE\OXYCODONE HYDROCHLORIDE:Dizziness:1.19), (ACITRETIN:Dizziness:0.13), (ALBIGLUTIDE:Dizziness: 0.5), (ALCOHOL:Dizziness:0.64), (ALEMTUZUMAB:Dizziness:0.66), (ALFUZOSIN:Dizziness: 0.19), (ALISKIREN HEMIFUMARATE:Dizziness:0.45), (ALOGLIPTIN BENZOATE:Dizziness:0.43), (AMINO ACIDS\DEXTROSE\ELECTROLYTES NOS:Dizziness:0.33), (AMISULPRIDE:Dizziness:0.69), (AMLODIPINE BESYLATE\ATORVASTATIN CALCIUM:Dizziness:0.53), (AMLODIPINE BESYLATE\HYDROCHLOROTHIAZIDE\OLMESARTAN MEDOXOMIL:Dizziness:0.41), (AMLODIPINE\VALSARTAN:Dizziness:0.46), (AMOXAPIN E:Dizziness:0.05), (AMOXICILLIN\CLARITHROMYCIN\ESOMEPRAZOLE:Dizziness:0.15), (AMPICILLIN SODIUM\SULBACTAM SODIUM:Dizziness:0.09), (ANTIHISTAMINES NOS:Dizziness:0.23), (ARIPIPRAZOLE LAUROXIL:Dizziness:0.06), (ATROPINE\DIPHENOXYLATE:Dizziness:0.04), (BECLOMET HASONE:Dizziness:0.03), (BENDAMUSTINE HYDROCHLORIDE:Dizziness:0.25), (BENSERAZIDE\LEVODOPA:Dizziness:0.3), (BEPRIDI L:Dizziness:0.42), (BETAMETHASONE DIPROPIONATE\CALCIPOTRIENE\CALCIPOTRIENE HYDRATE:Dizziness:0.38), (BRIVARACETAM:Dizziness:0.04), (BUPRENORPHINE HYDROCHLORIDE:Dizziness:0.67), (BUPRENORPHINE HYDROCHLORIDE\NALOXONE HYDROCHLORIDE:Dizziness:0.77), (CABOZANTINIB S-MALATE:Dizziness:0), (CALCITONIN:Dizziness:0.68), (CALCIUM LACTATE:Dizziness:0.25), (CALCIUM\MAGNESIUM:Dizziness:0.36), (CALCIUM\MAGNES IUM\ZINC:Dizziness:0.77), (CAPSAICIN:Dizziness:0.16), (CARFILZOMIB:Dizziness:1.11), (C EFAZOLIN:Dizziness:0.42), (CEFDITOREN PIVOXIL:Dizziness:0.28), (CETIRIZINE HYDROCHLORIDE\PSEUDOEPHEDRINE HYDROCHLORIDE:Dizziness:0.52), (CHLORHEXIDINE:Dizziness:0.42), (CILNIDIPINE:Diz ziness:0.5), (CODEINE PHOSPHATE\GUAIFENESIN:Dizziness:0.39), (CYPROHEPTADINE:Dizziness:0.52), (DACL ATASVIR DIHYDROCHLORIDE:Dizziness:0.02), (DALTEPARIN SODIUM:Dizziness:0.9), (DANTROLENE SODIUM:Dizziness:0.28), (DAPAGLIFLOZIN PROPANEDIOL:Dizziness:0.79), (DASABUVIR\OMBITASVIR\PARITAPREVIR\RITONAVI R:Dizziness:0.44), (DEGARELIX:Dizziness:0.13), (DEXTROAMPHETAMINE SULFATE:Dizziness:0.6), (DICLOFENAC SODIUM\MISOPROSTOL:Dizziness:0.2), (DIGITOXIN:Dizziness:0.21), (DILAZEP:Dizziness: 0.15), (DIPYRONE:Dizziness:0.81), (DOLASETRON MESYLATE:Dizziness:0.12), (DORZOLAMIDE HYDROCHLORIDE:Dizziness:0.36), (DOXYLAMINE SUCCINATE:Dizziness:0.07), (DULAGLUTIDE:Dizziness:0.26), (ECONAZOLE:Dizziness:0.21), (EDOXABAN TOSYLATE:Dizziness:1.77), (ELOTUZUMAB:Dizziness:0.35), (ELTROMBOPAG OLAMINE:Dizziness:0.03), (EMPAGLIFLOZIN\LINAGLIPTIN:Dizziness:0.87), (EPIRUBICI N:Dizziness:0.26), (ESOMEPRAZOLE MAGNESIUM\NAPROXEN:Dizziness:0.5), (ESTROGENS, CONJUGATED\MEDROXYPROGESTERONE ACETATE:Dizziness:0.93), (ESTRONE\SUS SCROFA OVARY:Dizziness:0.69), (ETHINYL ESTRADIOL\NORGESTIMATE:Dizziness:0.09), (ETHINYL ESTRADIOL\NORGESTREL:Dizziness:0.02), (ETRETINATE:Dizziness:0.75), (FENOTEROL HYDROBROMIDE\IPRATROPIUM BROMIDE:Dizziness:0.05), (FERROUS SULFATE\GLYCINE:Dizziness:0.16), (FLAVIN ADENINE DINUCLEOTIDE:Dizziness:0.01), (FLURAZEPAM\FLURAZEPAM HYDROCHLORIDE:Dizziness:0.17), (FLUVOXAMINE MALEATE:Dizziness:0.9), (FORMOTEROL FUMARATE\MOMETASONE FUROATE:Dizziness:0.33), (FURSULTIAMINE:Dizziness:0.01), (FUSIDIC ACID:Dizziness:1.21), (GADOVERSETAMIDE:Dizziness:0.22), (GENTAMICIN:Dizziness:0.35), (GIMERACIL\OTERACIL\TEGAFUR:Dizziness:0.81), (GLYCOPYRROLATE\INDACATE ROL MALEATE:Dizziness:0.19), (GLYCOPYRRONIUM\INDACATEROL:Dizziness:1.13), (GUAN FACINE:Dizziness:0.8), (HERBALS\SAW PALMETTO:Dizziness:0.01), (HYALURONATE SODIUM:Dizziness:0.86), (HYDROCHLOROTHIAZIDE\LOSARTAN:Dizziness:0.02), (HYDR OCODONE BITARTRATE\IBUPROFEN:Dizziness:0.51), (ILOPROST:Dizziness:1.36), (IMMUNE GLOBULIN NOS:Dizziness:0.11), (INDACATEROL:Dizziness:0.48), (INDAPAMIDE\PERINDOPRIL:Dizziness: 0.78), (INFLIXIMAB-DYYB: Dizziness:0.25), (INTERFERON:Dizziness:0.23), (INTERFERON ALFA-2A:Dizziness:0.19), (IRINOTECAN:Dizziness:0.22), (IRINOTECAN HYDROCHLORIDE:Dizziness:1.04), (IRSOGLADINE MALEATE:Dizziness:0.06), (IXAZOMIB:Dizziness:0.31), (LABETALOL HYDROCHLORIDE:Dizziness:0.78), (LAPATINIB DITOSYLATE:Dizziness:0.46), (LENVATINIB:Dizziness:0.16), (LEUCOVORIN CALCIUM:Dizziness:0.87), (LOMITAPIDE MESYLATE:Dizziness:0.92), (MEGESTROL:Dizziness:0.02), (MEPOLIZUMAB:Dizziness:0.22), (MESNA:Dizziness:0.03), (METFORMIN HYDROCHLORIDE\SAXAGLIPTIN HYDROCHLORIDE:Dizziness:0.01), (METHENOLONE ENANTHATE:Dizziness:0.21), (MEXILETINE HYDROCHLORIDE:Dizziness:0.15), (MIGLITOL:Dizziness:0.08), (MORPHINE SULFATE\NALTREXONE HYDROCHLORIDE:Dizziness:0.27), (NALOXONE:Dizziness:0.11), (NATEGLINIDE:Dizziness: 0.78), (OCTREOTIDE:Dizziness:0.1), (OLMESARTAN:Dizziness:0.13), (OMEPRAZOLE SODIUM:Dizziness:0.55), (OSIMERTINIB:Dizziness:0.05), (PANTOPRAZOLE MAGNESIUM:Dizziness:1.83), (PARICALCITOL:Dizziness:0.25), (PATIROMER:Dizziness:0.48), (PEMETREXED:Dizziness:0.48), (PENICILLIN G:Dizziness:0.14), (PENICILLIN V:Dizziness:0.08), (PETROLATUM:Dizziness:0.02), (PHENPROCOUMON:Dizziness:0.03), (P HYTONADIONE:Dizziness:0.56), (PIROXICAM:Dizziness:0.87), (POLYETHYLENE GLYCOL 4000:Dizziness:0.11), (PONATINIB:Dizziness:1.21), (POSACONAZOLE:Dizziness:0.22), (PRE DNISOLONE SODIUM PHOSPHATE:Dizziness:0.28), (PYRIDOXAL:Dizziness:0.08), (QUINACRINE:Dizziness:0.12), (QUINAPRIL:Dizziness:0.33), (REPAGLINIDE:Dizziness:0.34), (RETINOL:Dizziness:0.27), (RI ZATRIPTAN:Dizziness:0.5), (SALMETEROL:Dizziness:0.31), (SEVELAMER HYDROCHLORIDE:Dizziness:0.79), (SODIUM FLUORIDE:Dizziness:0.64), (SODIUM PHOSPHATE, DIBASIC\SODIUM PHOSPHATE, MONOBASIC:Dizziness:0.15), (SODIUM POLYSTYRENE SULFONATE:Dizziness:0.13), (SOFOSBUVIR\VELPATASVIR:Dizziness:0.16), (SUPLATAS T TOSILATE:Dizziness:0.18), (TEDUGLUTIDE\WATER:Dizziness:0.14), (TELOTRISTAT ETHYL:Dizziness:0.1), (TESTOSTERONE UNDECANOATE:Dizziness:0.23), (TETRACYCLINE:Dizziness:1.21), (THYROID, PORCINE\THYROID, UNSPECIFIED:Dizziness:0.21), (TINZAPARIN SODIUM:Dizziness:0.56), (TIPIRACIL HYDROCHLORIDE\TRIFLURIDINE:Dizziness:0.42), (TOBRAMYCIN:Dizziness:0.05), (TOL TERODINE:Dizziness:0.48), (TRANDOLAPRIL:Dizziness:0.32), (TRIMIPRAMINE MALEATE:Dizziness:0.16), (VEMURAFENIB:Dizziness:0.42), (VINORELBINE TARTRATE:Dizziness:0.09), (VORTIOXETINE HYDROBROMIDE:Dizziness:0.37), (ZICONOTIDE ACETATE:Dizziness:0.05), (ZILEUTON:Dizziness:0.31), (CALCIUM CARBONATE\CHOLECALCIFEROL:Drug abuse:0.51), (CERTOLIZUMAB PEGOL:Drug abuse:0.2), (DEXLANSOPRAZOLE:Drug abuse:0.65), (DILTIAZEM HYDROCHLORIDE:Drug abuse:0.55), (ENOXAPARIN:Drug abuse:0.05), (EVOLOCUMAB:Drug abuse:0.18), (FERROUS SULFATE:Drug abuse:0.49), (FISH OIL:Drug abuse:0.36), (IBRUTINIB:Drug abuse:0.58), (LORATADINE:Drug abuse:0.27), (LOVASTATIN:Drug abuse:0.25), (MAGNESIUM:Drug abuse:0.11), (METOPROLOL TARTRATE:Drug abuse:0.05), (MONTELUKAST SODIUM:Drug abuse:0.51), (PANTOPRAZOLE:Drug abuse:0.63), (PRAMIPEXOLE DIHYDROCHLORIDE:Drug abuse:0.21), (PRAVASTATIN SODIUM:Drug abuse:0.06), (RANOLAZINE:Drug abuse:0.05), (TAMSULOSIN:Drug abuse:0.09), (VARENICLINE TARTRATE:Drug abuse:1.18), (AMIODARONE HYDROCHLORIDE:Drug dependence:0.07), (ANASTROZOLE:Drug dependence:0.44), (ATAZANAVIR:Drug dependence:0.57), (AZITHROMYCIN DIHYDRATE:Drug dependence:0.31), (BUMETANIDE:Drug dependence:0.46), (CALCIUM CITRATE:Drug dependence:0.27), (CANDESARTAN:Drug dependence:0.22), (CANDESARTAN CILEXETIL\HYDROCHLOROTHIAZIDE:Drug dependence:0.09), (CERTOLIZUMAB PEGOL:Drug dependence:0.83), (CIPROFLOXACIN HYDROCHLORIDE:Drug dependence:0.61), (COLCHICINE:Drug dependence:0.13), (DALFAMPRIDINE:Drug dependence:0.77), (DASATINIB:Drug dependence:0.15), (DILTIAZEM HYDROCHLORIDE:Drug dependence:1.33), (DONEPEZIL:Drug dependence:0.02), (ELETRIPTAN HYDROBROMIDE:Drug dependence:0.61), (ENALAPRIL MALEATE:Drug dependence:0.13), (ENOXAPARIN:Drug dependence:0.66), (EVEROLIMUS:Drug dependence:0.37), (EVOLOCUMAB:Drug dependence:0.82), (EZETIMIBE:Drug dependence:0.21), (FLECAINIDE:Drug dependence:0.14), (FLUTICASONE:Drug dependence:0.25), (GUANFACINE HYDROCHLORIDE:Drug dependence:0.27), (IMATINIB MESYLATE:Drug dependence:0.24), (IMIPRAMINE HYDROCHLORIDE:Drug dependence:0.15), (INFLUENZA VIRUS VACCINE:Drug dependence:0.04), (IPRATROPIUM:Drug dependence:0.07), (LERCANIDIPINE HYDROCHLORIDE:Drug dependence:0.01), (MARAVIROC:Drug dependence:0.34), (METRONIDAZOLE\METRONIDAZOLE HYDROCHLORIDE:Drug dependence:0.46), (MOXIFLOXACIN HYDROCHLORIDE:Drug dependence:0.03), (NITROFURANTOIN\NITROFURANTOIN MONOHYDRATE:Drug dependence:0.23), (NORTRIPTYLINE HYDROCHLORIDE:Drug dependence:0.01), (NYSTATIN:Drug dependence:0.24), (PRAMIPEXOLE:Drug dependence:0.38), (PRAMIPEXOLE DIHYDROCHLORIDE:Drug dependence:0.53), (PRAVASTATIN:Drug dependence:0.56), (RANOLAZINE:Drug dependence:0.36), (RISEDRONATE SODIUM:Drug dependence:0.17), (RIZATRIPTAN BENZOATE:Drug dependence:0.61), (SACUBITRIL\VALSARTAN:Drug dependence:0.29), (SECUKINUMAB:Drug dependence:0.17), (SOTALOL:Drug dependence:0.52), (THALIDOMIDE:Drug dependence:0.01), (TOCILIZUMAB:Drug dependence:0.1), (UMECLIDINIUM BROMIDE\VILANTEROL TRIFENATATE:Drug dependence:0.22), (VERAPAMIL HYDROCHLORIDE:Drug dependence:0.78), (ACETAMINOPHEN\BUTALBITAL\CAFFEINE:Drug hypersensitivity:0.09), (AMIODARONE:Drug hypersensitivity:0.41), (AMPHETAMINE ASPARTATE\AMPHETAMINE SULFATE\DEXTROAMPHETAMINE SACCHARATE\DEXTROAMPHETAMINE SULFATE:Drug hypersensitivity:0.52), (CHOLESTYRAMINE:Drug hypersensitivity:0.16), (COLCHICINE:Drug hypersensitivity:0.21), (DALFAMPRIDINE:Drug hypersensitivity:0.89), (DICLOFENAC:Drug hypersensitivity:0.84), (DIVALPROEX SODIUM:Drug hypersensitivity:0.18), (DULOXETINE:Drug hypersensitivity:0.37), (ELETRIPTAN HYDROBROMIDE:Drug hypersensitivity:0.05), (ESZOPICLONE:Drug hypersensitivity:0.35), (FERROUS SULFATE:Drug hypersensitivity:0.75), (FERROUS SULFATE\FERROUS SULFATE, DRIED:Drug hypersensitivity:0.72), (FLUCONAZOLE:Drug hypersensitivity:0.47), (HYDROXYCHLOROQUINE DIPHOSPHATE:Drug hypersensitivity:0.15), (IBRUTINIB:Drug hypersensitivity:0.89), (ILOPROST:Drug hypersensitivity:0.32), (NIFEDIPINE:Drug hypersensitivity:0.2), (NILOTINIB:Drug hypersensitivity:0.04), (NORTRIPTYLINE:Drug hypersensitivity:0.01), (PANTOPRAZOLE:Drug hypersensitivity:0.87), (PAROXETINE:Drug hypersensitivity:0.18), (POLYETHYLENE GLYCOLS:Drug hypersensitivity:0.35), (RISEDRONATE SODIUM:Drug hypersensitivity:0.17), (SACUBITRIL\VALSARTAN:Drug hypersensitivity:0.17), (SECUKINUMAB:Drug hypersensitivity:0.29), (SUMATRIPTAN\SUMATRIPTAN SUCCINATE:Drug hypersensitivity:0.18), (TAMSULOSIN:Drug hypersensitivity:0.69), (TAMSULOSIN HYDROCHLORIDE:Drug hypersensitivity:0.17), (TOFACITINIB CITRATE:Drug hypersensitivity:0.37), (UMECLIDINIUM BROMIDE\VILANTEROL TRIFENATATE:Drug hypersensitivity:0.17), (VARENICLINE TARTRATE:Drug hypersensitivity:0.5), (VITAMIN B:Drug hypersensitivity:0.2), (WARFARIN:Drug hypersensitivity:1.21), (WARFARIN SODIUM:Drug hypersensitivity:1.21), (ETHAMBUTOL HYDROCHLORIDE:Drug reaction with eosinophilia and systemic symptoms:0.79), (.ALPHA.1-PROTEINASE INHIBITOR HUMAN:Drug therapy:0.03), (ACENOCOUMAROL:Drug therapy:0.19), (ACETAMINOPHEN\PROPOXYPHENE HYDROCHLORIDE:Drug therapy:0.02), (ACETAZOLAMIDE:Drug therapy:0.96), (ADO-TRASTUZUMAB EMTANSINE:Drug therapy:0.58), (AGALSIDASE BETA:Drug therapy:0.13), (ALUMINUM HYDROXIDE\DIMETHICONE\MAGNESIUM HYDROXIDE:Drug therapy:0.12), (AMISULPRIDE:Drug therapy:0.92), (ARIPIPRAZOLE LAUROXIL:Drug therapy:0.14), (AZILSARTAN KAMEDOXOMIL:Drug therapy:0.26), (BENAZEPRIL\BENAZEPRIL HYDROCHLORIDE:Drug therapy:0.45), (BISACODYL OR DOCUSATE SODIUM:Drug therapy:1.25), (BUDESONIDE\FORMOTEROL FUMARATE:Drug therapy:0.57), (CALCITONIN SALMON:Drug therapy:0.23), (CALCIUM CHLORIDE\DEXTROSE MONOHYDRATE\MAGNESIUM CHLORIDE\SODIUM CHLORIDE\SODIUM LACTATE:Drug therapy:0.06), (CANAKINUMAB:Drug therapy:0.53), (CANDESARTAN CILEXETIL\HYDROCHLOROTHIAZIDE:Drug therapy:1.55), (CARVEDILOL PHOSPHATE:Drug therapy:1.26), (CEFUROXIME AXETIL:Drug therapy:0.04), (CERTOLIZUMAB PEGOL:Drug therapy:1.52), (CETIRIZINE HYDROCHLORIDE\PSEUDOEPHEDRINE HYDROCHLORIDE:Drug therapy:0.15), (CETUXIMAB:Drug therapy:0.02), (CIMETIDINE:Drug therapy:0.61), (CLINDAMYCIN:Drug therapy:0.88), (CLOMIPRAMINE HYDROCHLORIDE:Drug therapy:0.23), (CYPROHEPTADINE:Drug therapy:0.03), (DACLIZUMAB:Drug therapy:0.85), (DANAZOL:Drug therapy:0.56), (DAPAGLIFLOZIN:Drug therapy:0.3), (DARATUMUMAB:Drug therapy:0.07), (DEXAMETHASONE ACETATE:Drug therapy:0.29), (DICLOFENAC:Drug therapy:1.71), (DIGITOXIN:Drug therapy:0.07), (DIPYRONE:Drug therapy:0.38), (DOCUSATE CALCIUM:Drug therapy:0.54), (DOFETILIDE:Drug therapy:0.73), (DOXEPIN HYDROCHLORIDE:Drug therapy:0.37), (DULAGLUTIDE:Drug therapy:0.42), (ECONAZOLE:Drug therapy:0.51), (EDOXABAN TOSYLATE:Drug therapy:0.93), (EVOLOCUMAB:Drug therapy:1.65), (EXEMESTANE:Drug therapy:0.37), (FENTANYL CITRATE:Drug therapy:0.53), (FLUOCINONIDE:Drug therapy:0.37), (FLUTICASONE FUROATE:Drug therapy:0.54), (FLUVOXAMINE MALEATE:Drug therapy:0.03), (FONDAPARINUX:Drug therapy:0.43), (FORMOTEROL:Drug therapy:0.15), (FORMOTEROL FUMARATE:Drug therapy:0.65), (FORMOTEROL FUMARATE\MOMETASONE FUROATE:Drug therapy:0.13), (FUSIDIC ACID:Drug therapy:0.19), (GLYCOPYRROLATE:Drug therapy:0.14), (GLYCOPYRRONIUM\INDACATEROL:Drug therapy:0.63), (GUANFACINE HYDROCHLORIDE:Drug therapy:0.28), (HERBALS\TURMERIC:Drug therapy:0.42), (HYDROCHLOROTHIAZIDE\LOSARTAN:Drug therapy:0.41), (HYDROCORTISONE ACETATE:Drug therapy:0.53), (HYDROXYCHLOROQUINE DIPHOSPHATE:Drug therapy:0.46), (ILOPROST:Drug therapy:0.33), (IMATINIB MESYLATE:Drug therapy:1.03), (INDAPAMIDE\PERINDOPRIL:Drug therapy:0.89), (IXAZOMIB:Drug therapy:0.12), (LACOSAMIDE:Drug therapy:0.17), (LERCANIDIPINE HYDROCHLORIDE:Drug therapy:0.87), (LIDOCAINE HYDROCHLORIDE:Drug therapy:0.56), (LIDOCAINE\PRILOCAINE:Drug therapy:0.37), (LINACLOTIDE:Drug therapy:0.02), (LOMITAPIDE MESYLATE:Drug therapy:0.16), (MECLIZINE HYDROCHLORIDE:Drug therapy:0.3), (MEPERIDINE:Drug therapy:0.08), (METAMIZOLE:Drug therapy:0.17), (METFORMIN HYDROCHLORIDE\VILDAGLIPTIN:Drug therapy:0.1), (METHENAMINE HIPPURATE:Drug therapy:0.48), (MICAFUNGIN:Drug therapy:0.08), (MIDODRINE:Drug therapy:0.4), (NADOLOL:Drug therapy:0.47), (NATEGLINIDE:Drug therapy:0.16), (NILOTINIB:Drug therapy:1.99), (NORTRIPTYLINE:Drug therapy:0.86), (OCTREOTIDE ACETATE:Drug therapy:0.34), (OSPEMIFENE:Drug therapy:0.16), (PARICALCITOL:Drug therapy:0.01), (PEGINTERFERON BETA-1A:Drug therapy:0.77), (PENICILLIN V:Drug therapy:0.04), (PENTOSAN POLYSULFATE SODIUM:Drug therapy:0.32), (PERTUZUMAB:Drug therapy:0.08), (PIROXICAM:Drug therapy:0.35), (PONATINIB:Drug therapy:0.77), (QUINAPRIL:Drug therapy:0.04), (RABEPRAZOLE:Drug therapy:0.7), (RADIATION THERAPY:Drug therapy:0.04), (RALOXIFENE HYDROCHLORIDE:Drug therapy:0.5), (RASBURICASE:Drug therapy:0.35), (RIVASTIGMINE:Drug therapy:0.19), (RIVASTIGMINE TARTRATE:Drug therapy:0.1), (RIZATRIPTAN:Drug therapy:0.02), (ROPINIROLE HYDROCHLORIDE:Drug therapy:0.7), (SALMETEROL XINAFOATE:Drug therapy:0.04), (SECUKINUMAB:Drug therapy:1.07), (SITAGLIPTIN:Drug therapy:0.13), (SODIUM OXYBATE:Drug therapy:1.53), (SULFAMETHOXAZOLE:Drug therapy:0.55), (SUMATRIPTAN\SUMATRIPTAN SUCCINATE:Drug therapy:1.51), (TERBINAFINE HYDROCHLORIDE:Drug therapy:0.36), (TESTOSTERONE UNDECANOATE:Drug therapy:0.06), (TETRACYCLINE:Drug therapy:0.91), (TOCOPHEROL:Drug therapy:0.08), (TRASTUZUMAB:Drug therapy:0.33), (TRIAMCINOLONE ACETONIDE:Drug therapy:1.5), (TROSPIUM CHLORIDE:Drug therapy:0.22), (VALGANCICLOVIR HYDROCHLORIDE:Drug therapy:0.55), (CETUXIMAB:Drug toxicity prophylaxis:0.34), (DEXLANSOPRAZOLE:Drug toxicity prophylaxis:0), (EVOLOCUMAB:Drug toxicity prophylaxis:0.17), (IRINOTECAN HYDROCHLORIDE:Drug toxicity prophylaxis:0.08),(ARMODAFINIL:Drug use disorder:0.19), (DEXLANSOPRAZOLE:Drug use disorder:0.61), (ENALAPRIL:Drug use disorder:0.3), (EVOLOCUMAB:Drug use disorder:0.09), (MAGNESIUM:Drug use disorder:0.07), (METRONIDAZOLE:Drug use disorder:0), (PAROXETINE\PAROXETINE HYDROCHLORIDE:Drug use disorder:0.01), (SODIUM OXYBATE:Drug use disorder:0.51), (TERIFLUNOMIDE:Drug use disorder:0.01), (.ALPHA.1-PROTEINASE INHIBITOR HUMAN:Dry eye:0.49), (ABOBOTULINUMTOXINA:Dry eye:0.42), (ACEBUTOLOL HYDROCHLORIDE:Dry eye:0.1), (ACETAMINOPHEN\\CAFFEINE:Dry eye:0.47), (ACETAMINOPHEN\PROPOXYPHENE NAPSYLATE:Dry eye:1.06), (ACRIVASTINE\PSEUDOEPHEDRINE HYDROCHLORIDE:Dry eye:0.12), (ALBIGLUTIDE:Dry eye:0.4), (ALCOHOL:Dry eye:0.74), (ALEMTUZUMAB:Dry eye:0.95), (ALOGLIPTIN BENZOATE:Dry eye:0.15), (AMIKACIN:Dry eye:0.2), (AMINO ACIDS\DEXTROSE\ELECTROLYTES NOS:Dry eye:0.25), (AMISULPRIDE:Dry eye:0.77), (AMLODIPINE BESYLATE\HYDROCHLOROTHIAZIDE\OLMESARTAN MEDOXOMIL:Dry eye:0.28), (AMLODIPINE BESYLATE\TELMISARTAN:Dry eye:0.03), (AMLODIPINE\PERINDOPRIL:Dry eye:0.19), (AMMONIUM CHLORIDE\DEXTROMETHORPHAN\SODIUM CITRATE:Dry eye:0.33), (AMOXAPINE:Dry eye:0.12), (AMOXICILLIN\CLARITHROMYCIN\ESOMEPRAZOLE:Dry eye:0.24), (ANAGRELIDE HYDROCHLORIDE:Dry eye:0.06), (ARIPIPRAZOLE LAUROXIL:Dry eye:0.05), (ATROPINE\DIPHENOXYLATE:Dry eye:0.22), (BENZOCAINE:Dry eye:0.1), (BEPRIDIL:Dry eye:0.04), (BETAMETHASONE DIPROPIONATE\CALCIPOTRIENE:Dry eye:0.66), (BETAMETHASONE DIPROPIONATE\CALCIPOTRIENE\CALCIPOTRIENE HYDRATE:Dry eye:1.09), (BETAMETHASONE SODIUM PHOSPHATE:Dry eye:0.21), (BETAMETHASONE\CALCIPOTRIENE:Dry eye:0.91), (BETHANECHOL CHLORIDE:Dry eye:0.32), (BILASTINE:Dry eye:0.03), (BISOPROLOL FUMARATE\HYDROCHLOROTHIAZIDE:Dry eye:0.56), (BROMFENAC\BROMFENAC SODIUM:Dry eye:0.58), (BUPIVACAINE HYDROCHLORIDE:Dry eye:0.24), (BUPRENORPHINE HYDROCHLORIDE:Dry eye:0.87), (BUPRENORPHINE HYDROCHLORIDE\NALOXONE HYDROCHLORIDE:Dry eye:1.22), (CABOZANTINIB S-MALATE:Dry eye:0.5), (CALCITONIN:Dry eye:0.98), (CALCIUM CARBONATE\ERGOCALCIFEROL\RETINOL:Dry eye:0.46), (CALCIUM\VITAMINS:Dry eye:0.1), (CANAGLIFLOZIN\METFORMIN HYDROCHLORIDE:Dry eye:1.44), (CANAKINUMAB:Dry eye:1.13), (CARBAMIDE PEROXIDE:Dry eye:0.59), (CARBIDOPA:Dry eye:0.59), (CASANTHRANOL\DOCUSATE SODIUM:Dry eye:0.64), (CERITINIB:Dry eye:0.62), (CHLORPHENIRAMINE\HYDROCODONE:Dry eye:0.84), (CILASTATIN SODIUM\IMIPENEM:Dry eye:1.4), (CINACALCET HYDROCHLORIDE:Dry eye:0.57), (CLIKSTAR:Dry eye:0.26), (CLOBAZAM:Dry eye:0.42), (CYCLOPENTOLATE:Dry eye:0.2), (CYPROHEPTADINE HYDROCHLORIDE:Dry eye:0.46), (DABRAFENIB MESYLATE:Dry eye:0.53), (DANAZOL:Dry eye:0.83), (DANTROLENE SODIUM:Dry eye:0.24), (DAPAGLIFLOZIN PROPANEDIOL\METFORMIN HYDROCHLORIDE:Dry eye:0.03), (DASATINIB:Dry eye:2.75), (DEFLAZACORT:Dry eye:1.69), (DEGARELIX:Dry eye:0.14), (DEXPANTHENOL\NIACINAMIDE\PYRIDOXINE HYDROCHLORIDE\RIBOFLAVIN 5′-PHOSPHATE SODIUM\THIAMINE HYDROCHLORIDE:Dry eye:0.01), (DIGITOXIN:Dry eye:0.28), (DIPYRONE\PROMETHAZINE HYDROCHLORIDE:Dry eye:0.26), (DOLASETRON MESYLATE:Dry eye:0.76), (DULAGLUTIDE:Dry eye:0.31), (DUTASTERIDE\TAMSULOSIN HYDROCHLORIDE:Dry eye:0.65), (ECONAZOLE:Dry eye:0.97), (ELBASVIR\GRAZOPREVIR:Dry eye:0.36), (ELTROMBOPAG OLAMINE:Dry eye:0.68), (EPERISONE HYDROCHLORIDE:Dry eye:0.13), (ERIBULIN MESYLATE:Dry eye:0.07), (ESOMEPRAZOLE SODIUM:Dry eye:0.1), (ETHACRYNIC ACID:Dry eye:0.18), (ETHINYL ESTRADIOL\NORGESTREL:Dry eye:0.36), (ETRETINATE:Dry eye:1.53), (FENOTEROL HYDROBROMIDE:Dry eye:0.38), (FERROUS BISGLYCINATE:Dry eye:0.02), (FERROUS SULFATE ANHYDROUS:Dry eye:0.38), (FEXOFENADINE HYDROCHLORIDE\PSEUDOEPHEDRINE HYDROCHLORIDE:Dry eye:0.02), (FLAVIN ADENINE DINUCLEOTIDE:Dry eye:0.03), (FLUINDIONE:Dry eye:0.71), (FLURAZEPAM\FLURAZEPAM HYDROCHLORIDE:Dry eye:0.01), (FLUTAMIDE:Dry eye:0.06), (FLUTICASONE FUROATE\SALMETEROL XINAFOATE:Dry eye:1.32), (FONDAPARINUX SODIUM:Dry eye:0.52), (FORMOTEROL FUMARATE\MOMETASONE FUROATE:Dry eye:0.77), (FOSPHENYTOIN\FOSPHENYTOIN SODIUM:Dry eye:0.08), (FROVATRIPTAN SUCCINATE:Dry eye:0.19), (GEFITINIB:Dry eye:0.33), (GEMCITABINE\GEMCITABINE HYDROCHLORIDE:Dry eye:0.12), (GIMERACIL\OTERACIL\TEGAFUR:Dry eye:1.03), (GINGER:Dry eye:0.29), (GLIPIZIDE\METFORMIN HYDROCHLORIDE:Dry eye:0.33), (GLUTAMINE:Dry eye:0.13), (GLYCOPYRROLATE\INDACATEROL MALEATE:Dry eye:0.38), (GLYCOPYRRONIUM:Dry eye:0.26), (GUANFACINE HYDROCHLORIDE:Dry eye:0.4), (HEPARIN CALCIUM:Dry eye:0.14), (HERBALS\SAW PALMETTO:Dry eye:0.55), (HYOSCYAMINE SULFATE\METHENAMINE\METHYLENE BLUE\PHENYL SALICYLATE\SODIUM PHOSPHATE, MONOBASIC, MONOHYDRATE:Dry eye:0.2), (IBRITUMOMAB TIUXETAN:Dry eye:0.34), (ILOPROST:Dry eye:0.68), (IMATINIB MESYLATE:Dry eye:1.95), (IMIGLUCERASE:Dry eye:0.2), (IMIPENEM:Dry eye:0.18), (IMIQUIMOD:Dry eye:0.33), (INDACATEROL:Dry eye:0.83), (INDAPAMIDE\PERINDOPRIL:Dry eye:1.64), (INFLIXIMAB-DYYB:Dry eye:0.81), (INTERFERON:Dry eye:1.08), (INTERFERON ALFA-2A:Dry eye:1.52), (INTERFERON GAMMA-1B:Dry eye:0.47), (IODINE:Dry eye:0.56), (IVABRADINE:Dry eye:0.18), (IVABRADINE HYDROCHLORIDE:Dry eye:0.05), (LANREOTIDE:Dry eye:0.13), (LANREOTIDE ACETATE:Dry eye:0.72), (LAPATINIB DITOSYLATE:Dry eye:1.01), (LENVATINIB:Dry eye:0.52), (LEVOLEUCOVORIN CALCIUM:Dry eye:0.29), (LOMITAPIDE MESYLATE:Dry eye:1), (LUTETIUM LU-177:Dry eye:0.01), (MAGNESIUM GLUCONATE:Dry eye:0.19), (MEGESTROL:Dry eye:0.44), (MEPOLIZUMAB:Dry eye:0.71), (METFORMIN HYDROCHLORIDE\SAXAGLIPTIN HYDROCHLORIDE:Dry eye:0.3), (METFORMIN HYDROCHLORIDE\VILDAGLIPTIN:Dry eye:0.82), (METHENAMINE MANDELATE:Dry eye:0.24), (METHENOLONE ENANTHATE:Dry eye:0.3), (METHYLPREDNISOLONE ACETATE:Dry eye:2.03), (MIDODRINE HYDROCHLORIDE:Dry eye:0.68), (NALOXONE HYDROCHLORIDE\TILIDINE HYDROCHLORIDE:Dry eye:0.87), (NAPROXEN SODIUM\SUMATRIPTAN SUCCINATE:Dry eye:0.03), (NICARDIPINE:Dry eye:0.47), (NOREPINEPHRINE:Dry eye:0.04), (OLMESARTAN:Dry eye:0.02), (OPIPRAMOL:Dry eye:0.23), (PANITUMUMAB:Dry eye:0.53), (PANTOPRAZOLE MAGNESIUM:Dry eye:2), (PARICALCITOL:Dry eye:0.32), (PATIROMER:Dry eye:0.56), (PENTAMIDINE ISETHIONATE:Dry eye:0.16), (PENTOSTATIN:Dry eye:0.48), (PERMETHRIN:Dry eye:0.17), (POLYMYXIN B SULFATE\TRIMETHOPRIM:Dry eye:0.12), (PRAMOXINE HYDROCHLORIDE:Dry eye:0.16), (PROTRIPTYLINE HYDROCHLORIDE:Dry eye:0.82), (RADIATION THERAPY:Dry eye:1.24), (RAMUCIRUMAB:Dry eye:0.62), (RILPIVIRINE HYDROCHLORIDE:Dry eye:0.1), (SACCHAROMYCES CEREVISIAE:Dry eye:0.9), (SALMETEROL:Dry eye:0.48), (SIBUTRAMINE HYDROCHLORIDE:Dry eye:0.05), (SOFOSBUVIR\VELPATASVIR:Dry eye:0.16), (SULFACETAMIDE SODIUM\SULFUR:Dry eye:0.04), (TASIMELTEON:Dry eye:0.11), (TAZOBACTAM:Dry eye:0.15), (TELOTRISTAT ETHYL:Dry eye:0.2), (TERBUTALINE SULFATE:Dry eye:1.98), (TESTOSTERONE UNDECANOATE:Dry eye:0.61), (TETRACAINE HYDROCHLORIDE:Dry eye:0.1), (THEOPHYLLINE:Dry eye:0.26), (TIGECYCLINE:Dry eye:0.09), (TIPIRACIL HYDROCHLORIDE\TRIFLURIDINE:Dry eye:0.73), (TRAMETINIB DIMETHYL SULFOXIDE:Dry eye:0.48), (TRIMIPRAMINE MALEATE:Dry eye:0.18), (TRIPTORELIN PAMOATE:Dry eye:0.56), (TROSPIUM CHLORIDE:Dry eye:1.14), (UBIQUINOL:Dry eye:0.06), (VALDECOXIB:Dry eye:0.15), (VINORELBINE TARTRATE:Dry eye:1), (WARFARIN POTASSIUM:Dry eye:0.27), (YEAST:Dry eye:0.63), (ZICONOTIDE ACETATE:Dry eye:0.03), (ALCOHOL:Dry mouth:0.09), (AMIODARONE HYDROCHLORIDE:Dry mouth:0.21), (ANASTROZOLE:Dry mouth:2.14), (AZITHROMYCIN DIHYDRATE:Dry mouth:0.55), (BENAZEPRIL\BENAZEPRIL HYDROCHLORIDE:Dry mouth:0.24), (BICALUTAMIDE:Dry mouth:0.56), (CALCIUM ACETATE:Dry mouth:0.18), (CANAKINUMAB:Dry mouth:0.09), (CANDESARTAN CILEXETIL:Dry mouth:1), (CARVEDILOL PHOSPHATE:Dry mouth:0.49), (CEFTRIAXONE SODIUM:Dry mouth:0.18), (CHLORTHALIDONE:Dry mouth:0.21), (CHONDROITIN SULFATE (BOVINE)\GLUCOSAMINE HYDROCHLORIDE:Dry mouth:0), (COD LIVER OIL:Dry mouth:0.13), (CORTICOTROPIN:Dry mouth:0.05), (DALFAMPRIDINE:Dry mouth:0.99), (DASATINIB:Dry mouth:1.13), (DIGOXIN:Dry mouth:1.04), (DOFETILIDE:Dry mouth:0.01), (DOXAZOSIN MESYLATE:Dry mouth:0.45), (EDOXABAN TOSYLATE:Dry mouth:0.11), (ENALAPRIL:Dry mouth:1.66), (EPIRUBICIN:Dry mouth:0.03), (EVOLOCUMAB:Dry mouth:1.02), (FEXOFENADINE\PSEUDOEPHEDRINE:Dry mouth:0.04), (FINGOLIMOD HYDROCHLORIDE:Dry mouth:0.59), (FLUOXETINE\FLUOXETINE HYDROCHLORIDE:Dry mouth:0.66), (FULVESTRANT:Dry mouth:0.7), (HYDROXYCHLOROQUINE DIPHOSPHATE:Dry mouth:0.33), (HYDROXYUREA:Dry mouth:0.27), (HYOSCYAMINE:Dry mouth:0.48), (INDAPAMIDE:Dry mouth:0.13), (ISOSORBIDE:Dry mouth:0.51), (LABETALOL\LABETALOL HYDROCHLORIDE:Dry mouth:0.62), (LACTASE:Dry mouth:0.05), (LEDIPASVIR\SOFOSBUVIR:Dry mouth:0.17), (LERCANIDIPINE:Dry mouth:0.01), (LERCANIDIPINE HYDROCHLORIDE:Dry mouth:0.02), (LINAGLIPTIN:Dry mouth:0.47), (LISDEXAMFETAMINE DIMESYLATE:Dry mouth:0.7), (LITHIUM CARBONATE:Dry mouth:0.79), (METHENAMINE HIPPURATE:Dry mouth:0.47), (METHYLPREDNISOLONE ACETATE:Dry mouth:0.46), (MOXIFLOXACIN HYDROCHLORIDE:Dry mouth:0.82), (NITROFURANTOIN\NITROFURANTOIN MONOHYDRATE:Dry mouth:0.07), (PANTOPRAZOLE MAGNESIUM:Dry mouth:0.62), (PAROXETINE:Dry mouth:0.84), (PAZOPANIB HYDROCHLORIDE:Dry mouth:0.62), (PEGINTERFERON BETA-1A:Dry mouth:0.36), (PENICILLIN:Dry mouth:0.6), (PERINDOPRIL:Dry mouth:0.01), (PHENYTOIN:Dry mouth:0.21), (PIROXICAM:Dry mouth:0.16), (PREDNISOLONE SODIUM PHOSPHATE:Dry mouth:0.01), (REGORAFENIB:Dry mouth:0.26), (ROFLUMILAST:Dry mouth:0.46), (SACUBITRIL\VALSARTAN:Dry mouth:0.91), (SOTALOL:Dry mouth:0.57), (SUNITINIB MALATE:Dry mouth:0.03), (TERBUTALINE SULFATE:Dry mouth:0.15), (TIMOLOL:Dry mouth:0.28), (UMECLIDINIUM BROMIDE\VILANTEROL TRIFENATATE:Dry mouth:0.41), (VANCOMYCIN HYDROCHLORIDE:Dry mouth:0.04), (VILAZODONE HYDROCHLORIDE:Dry mouth:0.32), (ZINC:Dry mouth:0.32), (ABIRATERONE ACETATE:Dry skin:0.07), (ACITRETIN:Dry skin:0), (ALCOHOL:Dry skin:0.59), (ALEMTUZUMAB:Dry skin:0.17), (ALFUZOSIN:Dry skin:0.04), (AMISULPRIDE:Dry skin:0.4), (AMLODIPINE BESYLATE\ATORVASTATIN CALCIUM:Dry skin:0.06), (AMOXAPINE:Dry skin:0.07), (ANASTROZOLE:Dry skin:2.1), (BENAZEPRIL\BENAZEPRIL HYDROCHLORIDE:Dry skin:0.19), (BETAMETHASONE DIPROPIONATE\CALCIPOTRIENE\CALCIPOTRIENE HYDRATE:Dry skin:0.06), (BUPRENORPHINE HYDROCHLORIDE\NALOXONE HYDROCHLORIDE:Dry skin:0.75), (CALCITRIOL:Dry skin:0.89), (CALCIUM ACETATE:Dry skin:0.53), (CALCIUM LACTATE:Dry skin:0.26), (CANAGLIFLOZIN\METFORMIN HYDROCHLORIDE:Dry skin:0.3), (CARBIMAZOLE:Dry skin:0.01), (CARISOPRODOL:Dry skin:0.27), (CARVEDILOL PHOSPHATE:Dry skin:0.55), (CEFOTAXIME SODIUM:Dry skin:0.09), (CEFUROXIME AXETIL:Dry skin:0.03), (CILASTATIN SODIUM\IMIPENEM:Dry skin:0.85), (CILNIDIPINE:Dry skin:0.03), (CORTISONE\HYDROCORTISONE:Dry skin:0.83), (DABIGATRAN:Dry skin:0.01), (DABRAFENIB:Dry skin:0.7), (DANAZOL:Dry skin:0.25), (DAPAGLIFLOZIN PROPANEDIOL:Dry skin:0.2), (DASATINIB:Dry skin:1.55), (DEFLAZACORT:Dry skin:0.38), (DEVICE:Dry skin:0.37), (DILAZEP:Dry skin:0.01), (DIMENHYDRINATE:Dry skin:0.75), (DIPYRONE:Dry skin:0.06), (DOFETILIDE:Dry skin:0.2), (DOLASETRON MESYLATE:Dry skin:0.06), (DORZOLAMIDE:Dry skin:0.04), (EDOXABAN TOSILATE MONOHYDRATE:Dry skin:0.25), (ELETRIPTAN HYDROBROMIDE:Dry skin:0.5), (ELOTUZUMAB:Dry skin:0.33), (ENALAPRIL:Dry skin:2.27), (ESTROGENS, CONJUGATED\MEDROXYPROGESTERONE ACETATE:Dry skin:0.68), (ETRETINATE:Dry skin:0.59), (EVOLOCUMAB:Dry skin:0.64), (FESOTERODINE FUMARATE:Dry skin:0.85), (FLAVIN ADENINE DINUCLEOTIDE:Dry skin:0.17), (FLECAINIDE:Dry skin:0.67), (FLUDROCORTISONE ACETATE:Dry skin:0.26), (FLUOXETINE\FLUOXETINE HYDROCHLORIDE:Dry skin:0.72), (FLUVOXAMINE MALEATE:Dry skin:0.08), (FORMOTEROL FUMARATE:Dry skin:0.29), (FORMOTEROL FUMARATE DIHYDRATE\MOMETASONE FUROATE:Dry skin:0.57), (FULVESTRANT:Dry skin:0.5), (GEMCITABINE HYDROCHLORIDE:Dry skin:0.07), (GLUCOSAMINE:Dry skin:0.08), (HERBALS\SAW PALMETTO:Dry skin:0.01), (HERBALS\TURMERIC:Dry skin:0.36), (HYDROCORTISONE ACETATE:Dry skin:0.51), (HYOSCYAMINE:Dry skin:0.04), (IBRITUMOMAB TIUXETAN:Dry skin:0.18), (IMIPRAMINE HYDROCHLORIDE:Dry skin:0.69), (IMIQUIMOD:Dry skin:0.41), (INDAPAMIDE\PERINDOPRIL:Dry skin:0.88), (INFLIXIMAB-DYYB:Dry skin:0.38), (INTERFERON ALFA-2B:Dry skin:0.02), (ISOSORBIDE:Dry skin:0.81), (ITRACONAZOLE:Dry skin:0.64), (IXAZOMIB:Dry skin:0.58), (LABETALOL\LABETALOL HYDROCHLORIDE:Dry skin:0.83), (LACTOBACILLUS RHAMNOSUS:Dry skin:0.05), (LEVALBUTEROL HYDROCHLORIDE:Dry skin:0.9), (MESNA:Dry skin:0.69), (METFORMIN HYDROCHLORIDE\VILDAGLIPTIN:Dry skin:0.22), (METHENAMINE HIPPURATE:Dry skin:0.34), (METHENOLONE ENANTHATE:Dry skin:0.41), (METHYLPREDNISOLONE ACETATE:Dry skin:0.58), (MORPHINE SULFATE\NALTREXONE HYDROCHLORIDE:Dry skin:0.19), (NADOLOL:Dry skin:0.31), (NALOXONE HYDROCHLORIDE\OXYCODONE HYDROCHLORIDE:Dry skin:0.9), (NALOXONE HYDROCHLORIDE\TILIDINE HYDROCHLORIDE:Dry skin:0.12), (NATEGLINIDE:Dry skin:0.15), (NEBIVOLOL HYDROCHLORIDE:Dry skin:0.79), (NIRAPARIB:Dry skin:0.14), (NORETHINDRONE:Dry skin:0.06), (OLAPARIB:Dry skin:0.28), (OLODATEROL HYDROCHLORIDE\TIOTROPIUM BROMIDE MONOHYDRATE:Dry skin:0.56), (OXCARBAZEPINE:Dry skin:1.87), (PALBOCICLIB:Dry skin:0.14), (PAZOPANIB HYDROCHLORIDE:Dry skin:0.53), (PEGINTERFERON ALFA-2B:Dry skin:0.32), (PENICILLIN:Dry skin:0.53), (PENICILLIN G:Dry skin:0.04), (PENICILLIN V:Dry skin:0.13), (PENTOXIFYLLINE:Dry skin:0.01), (PHENOBARBITAL:Dry skin:0.87), (PIROXICAM:Dry skin:0.26), (POSACONAZOLE:Dry skin:0.05), (PREDNISOLONE ACETATE:Dry skin:0.89), (PREDNISOLONE SODIUM PHOSPHATE:Dry skin:0.24), (RIVASTIGMINE:Dry skin:0.25), (RIZATRIPTAN BENZOATE:Dry skin:0.86), (ROFLUMILAST:Dry skin:0.89), (SACUBITRIL\VALSARTAN:Dry skin:0.91), (SARPOGRELATE:Dry skin:0.19), (SODIUM OXYBATE:Dry skin:0.77), (TELMISARTAN:Dry skin:1.61), (TERAZOSIN HYDROCHLORIDE:Dry skin:0.02), (TERBUTALINE SULFATE:Dry skin:0.31), (THALIDOMIDE:Dry skin:1.98), (TIMOLOL:Dry skin:0.98), (TRICHLORMETHIAZIDE:Dry skin:0.2), (TROSPIUM CHLORIDE:Dry skin:0.03), (UMECLIDINIUM BROMIDE\VILANTEROL TRIFENATATE:Dry skin:0.28), (VANCOMYCIN HYDROCHLORIDE:Dry skin:0.65), (VARENICLINE TARTRATE:Dry skin:1.39), (VINORELBINE TARTRATE:Dry skin:0.18), (VITAMIN A:Dry skin:0.28), (ZINC:Dry skin:0.28), (ZONISAMIDE:Dry skin:0.2), (BEVACIZUMAB:Dry skin prophylaxis:0.08), (ACARBOSE:Duodenal ulcer:0.03), (ALBUTEROL SULFATE\IPRATROPIUM BROMIDE:Duodenal ulcer:0.06), (ANASTROZOLE:Duodenal ulcer:0.6), (BROMAZEPAM:Duodenal ulcer:0.19), (BUPROPION:Duodenal ulcer:0.03), (CALCIUM\VITAMIN D:Duodenal ulcer:0.45), (CEPHALEXIN:Duodenal ulcer:0.66), (CIPROFLOXACIN HYDROCHLORIDE:Duodenal ulcer:0.26), (CLONIDINE:Duodenal ulcer:0.27), (CLOTRIMAZOLE:Duodenal ulcer:0.03), (CORTISONE\HYDROCORTISONE:Duodenal ulcer:0.38), (DABIGATRAN:Duodenal ulcer:0), (DASATINIB:Duodenal ulcer:0.65), (DIPHENHYDRAMINE:Duodenal ulcer:0.7), (EDOXABAN TOSYLATE:Duodenal ulcer:0.08), (ENALAPRIL MALEATE:Duodenal ulcer:0.51), (ERYTHROPOIETIN:Duodenal ulcer:0.54), (EVOLOCUMAB:Duodenal ulcer:0.71), (EZETIMIBE\SIMVASTATIN:Duodenal ulcer:0.01), (FLECAINIDE:Duodenal ulcer:0.17), (GLYBURIDE:Duodenal ulcer:0.19), (HEPARIN SODIUM:Duodenal ulcer:0.58), (HYDRALAZINE HYDROCHLORIDE:Duodenal ulcer:0.08), (HYDROMORPHONE:Duodenal ulcer:0.1), (HYDROXYCHLOROQUINE SULFATE:Duodenal ulcer:0.67), (IBRUTINIB:Duodenal ulcer:1.12), (INSULIN DETEMIR:Duodenal ulcer:0.59), (IRINOTECAN HYDROCHLORIDE:Duodenal ulcer:0.12), (ISOSORBIDE:Duodenal ulcer:0.21), (LEUCOVORIN CALCIUM:Duodenal ulcer:0.66), (LITHIUM CARBONATE:Duodenal ulcer:0.44), (METHYLPREDNISOLONE ACETATE:Duodenal ulcer:0.26), (METRONIDAZOLE\METRONIDAZOLE HYDROCHLORIDE:Duodenal ulcer:0.22), (MOXIFLOXACIN HYDROCHLORIDE:Duodenal ulcer:0.03), (NALOXONE HYDROCHLORIDE\OXYCODONE HYDROCHLORIDE:Duodenal ulcer:0.06), (OMEGA-3-ACID ETHYL ESTERS:Duodenal ulcer:0.08), (OXYGEN:Duodenal ulcer:0.08), (PANTOPRAZOLE MAGNESIUM:Duodenal ulcer:0.06), (PAROXETINE:Duodenal ulcer:0.04), (PAROXETINE\PAROXETINE HYDROCHLORIDE:Duodenal ulcer:0.1), (PAZOPANIB HYDROCHLORIDE:Duodenal ulcer:0.12), (PRAMIPEXOLE DIHYDROCHLORIDE:Duodenal ulcer:0.17), (PROBIOTICS NOS:Duodenal ulcer:0.14), (RANOLAZINE:Duodenal ulcer:0.47), (SACUBITRIL\VALSARTAN:Duodenal ulcer:0.44), (SOTALOL:Duodenal ulcer:0.51), (THALIDOMIDE:Duodenal ulcer:0.15), (VITAMIN B:Duodenal ulcer:0.06), (IBRUTINIB:Dwarfism:0.18), (TOPIRAMATE:Dwarfism:0.47), (RIFAMPIN:Dyshidrotic eczema:0.65), (AMIODARONE:Dyskinesia:0.78), (AMIODARONE HYDROCHLORIDE:Dyskinesia:0.18), (AMOXICILLIN:Dyskinesia:0.3),(ARMODAFINIL: Dyskinesia:0.89), (CALCIUM CARBONATE\CHOLECALCIFEROL:Dyskinesia:0.6), (CANDESARTAN CILEXETIL:Dyskinesia:0.08), (CARISOPRODOL:Dyskinesia:0.2), (CARVEDILOL PHOSPHATE:Dyskinesia:0.22), (CERTOLIZUMAB PEGOL:Dyskinesia:0.23), (CLINDAMYCIN\CLINDAMYCIN PHOSPHATE:Dyskinesia:0.1), (COLCHICINE:Dyskinesia:0), (DIPHENHYDRAMINE:Dyskinesia: 0.35), (ENALAPRIL MALEATE:Dyskinesia:0.19), (ENOXAPARIN:Dyskinesia:0.37), (EVOLOCUMAB:Dyskinesia: 0.75), (FERROUS SULFATE:Dyskinesia:0.41), (FERROUS SULFATE\FERROUS SULFATE, DRIED:Dyskinesia:0.69), (FINGOLIMOD HYDROCHLORIDE:Dyskinesia:0.24), (FLUOXETINE\FLUOXETINE HYDROCHLORIDE:Dyskinesia:0.34), (ISTRADEFYLLINE:Dyskinesia:0.04), (METHYLPRE DNISOLONE SODIUM SUCCINATE:Dyskinesia:0.46), (METRONIDAZOLE:Dyskinesia:0.37), (OXYGEN:Dyskinesia: 0.33), (PROMETHAZINE HYDROCHLORIDE:Dyskinesia:0.03), (PYRAZINAMIDE:Dyskinesia:0.63), (SACUBITRIL\V ALSARTAN:Dyskinesia:0.28), (SCOPOLAMINE:Dyskinesia:0.06), (SELEGILINE HYDROCHLORIDE:Dyskinesia:0.12), (SOTALOL:Dyskinesia:0.14), (SUMATRIPTAN\SUMA TRIPTAN SUCCINATE:Dyskinesia:0.18), (TELMISARTAN:Dyskinesia:0), (TERIFLUNOMIDE:Dyskinesia: 0.58), (UMECLIDINIUM BROMIDE\VILANTEROL TRIFENATATE:Dyskinesia:0.04), (VARENICLINE TARTRATE:Dyskinesia:1.39), (.ALPHA.1-PROTEINASE INHIBITOR HUMAN:Dyslipidaemia:0.6), (.BETA.-ACETYLDIGOXIN:Dyslipidaemia:0.45), (ABACAVIR\LAMIVUDINE:Dyslipidaemia:0.03), (A CAMPROSATE:Dyslipidaemia:0.06), (ACETAMINOPHEN\BROMISOVALDIHYDROCODE INE PHOSPHATE\DIPHENHYDRAMINE SALICYLATEDYPHYLLINE\METHYLEPHEDRINE HYDROCHLORIDE, (+/−)-:Dyslipidaemia:0.1), (ACETAMINOPHENCAFFEINE:Dyslipidaemia:0.75), (ACETAMIN OPHEN\DIPHENHYDRAMINE HYDROCHLORIDE:Dyslipidaemia:0.13), (ACETAMINOPHEN\OXYCODONE\OXYCODON E HYDROCHLORIDE:Dyslipidaemia:1.61), (ACETAMINOPHENPROPOXYPHENE HYDROCHLORIDE:Dyslipidaemia:0.59), (ACLIDINIUM BROMIDE\FORMOTEROL FUMARATE:Dyslipidaemia:0.1), (ADUCANUMAB:Dyslipidaemia:0.2), (ALDESLEUKIN: Dyslipidaemia:0.25), (ALECTINIB:Dyslipidaemia:0.04), (ALUMINUM HYDROXIDE\MAGNESIUM OXIDE\OXETHAZAINE:Dyslipidaemia:0.29), (ALUMINUM SILICATE:Dyslipidaemia:0.82), (AMIKACIN SULFATE:Dyslipidaemia:0.71), (AMINO ACIDS\CARBOHYDRATES\MINERALS\SOYBEAN OIL\VITAMINS:Dyslipidaemia:0.38), (AMINOSALICYLIC ACID:Dyslipidaemia:0.26), (AMMONIUM LACTATE:Dyslipidaemia:0.48), (AMPICILLIN SODIUM:Dyslipidaemia:0.03), (ANAGRELIDE:Dyslipidaemia:0.16), (ANTIHISTAMINES NOS:Dyslipidaemia:0.23), (AROTINOLOL:Dyslipidaemia:0.3), (ASCORBIC ACID\BIOTIN\CALCIUM PANTOTHENATE\COBALAMIN\FOLIC ACID\NIACINAMIDE\PYRIDOXINE HYDROCHLORIDE\RIBOFLAVIN\THIAMINE MONONITRATE:Dyslipidaemia:0.43), (ASCORBIC ACID\BIOTINCYANOCOBALAMIN\FOLIC ACID\NIACIN\PANTOTHENIC ACID\PYRIDOXINE\RIBOFLAVIN\THIAMINE:Dyslipidaemia:0.03), (ASPIRIN\DIHYDROX YALUMINUM AMINOACETATE\MAGNESIUM CARBONATE:Dyslipidaemia:0.08), (ATAZANAVIR:Dyslipidaemia:0.56), (ATAZANAVIR\RI TONAVIR:Dyslipidaemia:0.04), (ATROPINE SULFATE:Dyslipidaemia:0.74), (AZELASTINE:Dyslipidaemia:0.4), (BACITRACIN:Dyslipidaemia: 0.28), (BECLOMETHASONE DIPROPIONATE\FORMOTEROL:Dyslipidaemia:1.05), (BETAMETHASONE DIPROPIONATE\CALCIPOTRIENE\CALCIPOTRIENE HYDRATE:Dyslipidaemia:0.61), (BETAMETHASONE DIPROPIONATE\CLOTRIMAZOLE:Dyslipidaemia:0.65), (BETAMETHASONE VALERATE\GENTAMICIN SULFATE:Dyslipidaemia:0.51), (BICARBONATE ION\POLYETHYLENE GLYCOLS\POTASSIUM CATION\SODIUM CHLORIDE\SODIUM SULFATE ANHYDROUS:Dyslipidaemia:0.1), (BIPERIDEN:Dyslipidaemia:0.49), (BLINATUMOMAB: Dyslipidaemia:0.19), (BOTULINUM TOXIN TYPE A:Dyslipidaemia:0.63), (BROMHEXINE:Dyslipidaemia:0.1), (BROMPERIDOL:Dyslipidaemia: 0.26), (BUDESONIDE\FORMOTEROL FUMARATE:Dyslipidaemia:1.87), (BUPRENORPHINE HYDROCHLORIDE\NALOXONE HYDROCHLORIDE:Dyslipidaemia:0.64), (CABOZANTINIB:Dyslipidaemia:0.11), (CALCITO NIN:Dyslipidaemia:0.7), (CALCITONIN SALMON:Dyslipidaemia:0.94), (CALCIUM GLYCEROPHOSPHATE\POLYETHYLENE GLYCOLS\POWDERED CELLULOSE\SACCHARIN SODIUM DIHYDRATE\SODIUM HYDROXIDE\SODIUM LAURYL SULFATE\SODIUM MAGNESIUM SILICATE\SODIUM MONOFLUOROPHOSPHATE\SORBITOL\WATER:Dyslipidaemia:0.1), (CALCIUM\MAGNE SIUM:Dyslipidaemia:0.67), (CALCIUM\MAGNESIUM\ZINC:Dyslipidaemia:0.59), (CARBAS ALATE:Dyslipidaemia:0.25), (CARBOXYMETHYLCELLULOSE SODIUM:Dyslipidaemia:0.3), (CARTEOLOL:Dyslipidaemia:0.32), (CASPOFUNGIN:Dyslipidaemia: 0.43), (CEFOTAXIME\CEFOTAXIME SODIUM:Dyslipidaemia:0.85), (CEFOTIAM HYDROCHLORIDE:Dyslipidaemia:0.11), (CEPHARANTHINE:Dyslipidaemia:0.68), (CETIRIZ INE HYDROCHLORIDE\PSEUDOEPHEDRINE HYDROCHLORIDE:Dyslipidaemia:0.11), (CHLORPHENIRAMINE\HYDROCODONE:Dyslipidaemia: 0.23), (CHLORPROMAZINE\PROMETHAZINE:Dyslipidaemia:0.32), (CHOLINE BITARTRATE\CYSTEINE\INOSITOL\MAMMAL LIVER:Dyslipidaemia:0.34), (CLIKSTAR:Dyslipidaemia:0.7), (COD LIVER OIL:Dyslipidaemia:0.25), (COLESTIPOL:Dyslipidaemia:0.02), (CORTICOTROPIN:Dyslipidaemia: 0.8), (CYANAMIDE:Dyslipidaemia:0.37), (CYPROHEPTADINE:Dyslipidaemia:0.38), (DA CLIZUMAB:Dyslipidaemia:1.31), (DANAZOL:Dyslipidaemia:1.07), (DEGARELIX:Dyslipidaemia: 1.19), (DEQUALINIUM CHLORIDE:Dyslipidaemia:0.24), (DEXAMETHASONE\OXYTETRACYCLINE:Dyslipidaemia: 0.01), (DEXTRAN:Dyslipidaemia:0.02), (DEXTROAMPHETAMINE SULFATE:Dyslipidaemia:0.04), (DIGITALIS:Dyslipidaemia:0.31), (DIGITOXIN:Dyslipidaemia: 2), (DOLASETRON MESYLATE:Dyslipidaemia:0.44), (DOPAMINE HYDROCHLORIDE:Dyslipidaemia:0.41), (DOXYLAMINE SUCCINATE:Dyslipidaemia:0.17), (DRONABINOL:Dyslipidaemia:1.7), (DUTASTERIDE\TA MSULOSIN HYDROCHLORIDE:Dyslipidaemia:1.79), (ECABET SODIUM:Dyslipidaemia:1.17), (ELBASVIR\GRAZOPREVIR:Dyslipidaemia:0.35), (ELTROM BOPAG OLAMINE:Dyslipidaemia:0.07), (EMTRICITABINE\RILPIVIRINE HYDROCHLORIDE\TENOFOVIR DISOPROXIL FUMARATE:Dyslipidaemia:0), (EMTRICITABINE\TENOFOVIR:Dyslipidaemia:0.25), (EPRA ZINONE:Dyslipidaemia:0.11), (ERIBULIN MESYLATE:Dyslipidaemia:0.26), (ERYTHROMYCIN\SULFISOXAZOLE:Dyslipidaemia:0.25), (ESMOLOL HYDROCHLORIDE:Dyslipidaemia:0.11), (ESTRIOL:Dyslipidaemia:0.09), (ESTRONE\SUS SCROFA OVARY:Dyslipidaemia:0.25), (ETRETINATE:Dyslipidaemia:2.17), (FERROUS GLYCINE SULFATE:Dyslipidaemia:0.59), (FESOTERODINE:Dyslipidaemia:0.02), (FEXOFENADINE\PSEUDOEPHEDRINE: Dyslipidaemia:0.31), (FLUOCINOLONE ACETONIDE:Dyslipidaemia:0.31), (FLUPHENAZINE:Dyslipidaemia:0.21), (FLURAZEPAM\FLURAZEPAM HYDROCHLORIDE:Dyslipidaemia:0.41), (FLURBIPROFEN AXETIL:Dyslipidaemia:0.18), (FLUTICASONE PROPIONATE\FORMOTEROL FUMARATE:Dyslipidaemia:0.69), (FURSULTIAMINE:Dyslipidaemia:1.89), (GIMERACIL\OTERACIL\TEGAFUR:Dyslipidaemia:1.36), (GLIMEPIRIDE\METFORMIN:Dyslipidaemia:0.05), (GLIMEPIRIDE\ROSIGLITAZONE MALEATE:Dyslipidaemia:0.81), (GLIPIZIDE\METFORMIN HYDROCHLORIDE:Dyslipidaemia:0.07), (GUANFACINE:Dyslipidaemia:0.26), (HYDROCHLOROTHIAZIDE\METOPROLOL SUCCINATE:Dyslipidaemia:1.25), (HYDROCODONE BITARTRATE\IBUPROFEN:Dyslipidaemia:0.21), (HYDROXYCHLOROQUINE DIPHOSPHATE:Dyslipidaemia:0.26), (HYOSCYAMINE:Dyslipidaemia:0.29), (IBRITUMOMAB TIUXETAN:Dyslipidaemia:0.89), (IBUDILAST:Dyslipidaemia:0.7), (IMIGLUCERASE:Dyslipidaemia: 0.78), (IMMUNE GLOBULIN NOS:Dyslipidaemia:0.26), (INFLIXIMAB-DYYB:Dyslipidaemia:1.27), (INSULIN PORK\INSULIN PURIFIED PORK:Dyslipidaemia:0.62), (INTERFERON:Dyslipidaemia:1.64), (INTERFERON ALFA-2A:Dyslipidaemia:1.46), (IODINE:Dyslipidaemia:0.56), (IOPAMIDOL:Dyslipidaemia:0.82), (IRINOTECAN HYDROCHLORIDE:Dyslipidaemia:1.37), (ISOLEUCINE\LEUCINE\VALINE:Dyslipidaemia: 0.44), (KETAMINE HYDROCHLORIDE:Dyslipidaemia:0.1), (LACTASE:Dyslipidaemia:0.03), (LACTITOL:Dyslipidaemia: 0.1), (LANDIOLOL HYDROCHLORIDE:Dyslipidaemia:0.73), (LANREOTIDE:Dyslipidaemia:0.37), (LENVATINI B:Dyslipidaemia:0.45), (LEVALBUTEROL:Dyslipidaemia:0.19), (LEVOCARNITINE HYDROCHLORIDE:Dyslipidaemia:1.5), (LEVOTHYROXINE SODIUM\POTASSIUM IODIDE:Dyslipidaemia:0.48), (LINACLOTIDE:Dyslipidaemia:0.12), (LINSIDOMINE:Dyslipidaemia: 1), (LIOTHYRONINE SODIUM:Dyslipidaemia:0.8), (LISDEXAMFETAMINE DIMESYLATE:Dyslipidaemia:0.54), (LOPINAVIR:Dyslipidaemia:0.68), (LUTETIUM LU-177:Dyslipidaemia:0.28), (MAGNESIUM ASPARTATE\POTASSIUM ASPARTATE:Dyslipidaemia:0.61), (MAGNESIUM PIDOLATE:Dyslipidaemia:0.24), (MEDROXYPROGESTERONE:Dyslipidaemia:0.05), (MEPE NZOLATE BROMIDE:Dyslipidaemia:0.64), (MEPERIDINE:Dyslipidaemia:1.44), (MEPOLIZUMAB:Dyslipidaemia: 0.84), (MESNA:Dyslipidaemia:0.69), (METFORMIN HYDROCHLORIDE\ROSIGLITAZONE MALEATE:Dyslipidaemia:1.08), (METHENAMINE HIPPURATE:Dyslipidaemia:1.38), (METHENOLONE ENANTHATE:Dyslipidaemia:1.44), (METHYLPHENIDATE HYDROCHLORIDE:Dyslipidaemia:1.02), (MIRIPLATIN:Dyslipidaemia:0.12), (MOEXIPRIL HYDROCHLORIDE:Dyslipidaemia:1.18), (MOMETASONE:Dyslipidaemia:0.03), (MORPHIN E SULFATE\NALTREXONE HYDROCHLORIDE:Dyslipidaemia:0.68), (MOSAPRIDE CITRATE DIHYDRATE:Dyslipidaemia:0.5), (NAFAMOSTAT MESYLATE:Dyslipidaemia:0.38), (NALDEMEDINE:Dyslipidaemia:0.04), (NALFURAFINE HYDROCHLORIDE:Dyslipidaemia:0.32), (NALTREXONE:Dyslipidaemia:0.31), (ONABOTUL INUMTOXINA:Dyslipidaemia:0.69), (ORPHENADRINE:Dyslipidaemia:0.36), (OXYBUTYNI N\OXYBUTYNIN CHLORIDE:Dyslipidaemia:0.48), (OXYMETAZOLINE:Dyslipidaemia:0.18), (PATIROMER: Dyslipidaemia:0.81), (PEGINTERFERON BETA-1A:Dyslipidaemia:1.22), (PENICILLIN G:Dyslipidaemia:0.45), (PENTAERYTHRITOL TETRANITRATE:Dyslipidaemia:0.32), (PENTOSTATIN:Dyslipidaemia:0.07), (PHENOTHIAZ INE:Dyslipidaemia:0.42), (PIMECROLIMUS:Dyslipidaemia:0.47), (PIRITRAMIDE:Dyslipidaemia: 0.97), (PIZOTYLINE:Dyslipidaemia:0.02), (POLYETHYLENE GLYCOL 3350\POTASSIUM CHLORIDE\SODIUM BICARBONATE\SODIUM CHLORIDE\SODIUM SULFATE ANHYDROUS:Dyslipidaemia:0.73), (POTASSIUM IODIDE:Dyslipidaemia:0.58), (PREDNISOLONE SODIUM PHOSPHATE:Dyslipidaemia:0.28), (PREDNISOLONE SODIUM SUCCINATE:Dyslipidaemia:1.42), (PREDNISOLONE VALERATE ACETATE:Dyslipidaemia:0.39), (PROMAZINE HYDROCHLORIDE:Dyslipidaemia:0.34), (PROPAFENONE HYDROCHLORIDE:Dyslipidaemia:1.36), (PYRIDOXAL PHOSPHATE:Dyslipidaemia:0.19), (QUERCUS SALICINA STEM:Dyslipidaemia:0.25), (QUINACRINE:Dyslipidaemia:0.46), (RADIATION THERAPY:Dyslipidaemia:0.97), (RHUBARB:Dyslipidaemia:0.14), (RIZATRIPTAN:Dyslipidaemia: 0.07), (ROSIGLITAZONE:Dyslipidaemia:0.36), (SCOPOLAMINE HYDROBROMIDE:Dyslipidaemia:0.29), (SENNOSIDE B:Dyslipidaemia:0.06), (SERRAPEPTASE:Dyslipidaemia:0.13), (SITAFLOXACIN:Dyslipidaemia: 0.57), (SIVELESTAT SODIUM:Dyslipidaemia:0.33), (SODIUM FLUORIDE:Dyslipidaemia:0.95), (SOFOSBUVIR\VELPATASVIR:Dyslipidaemia:0.58), (STRE PTOMYCIN SULFATE:Dyslipidaemia:0.3), (SUMATRIPTAN\SUMATRIPTAN SUCCINATE:Dyslipidaemia:1.04), (TAMOXIFEN CITRATE:Dyslipidaemia:0.31), (TANDOSPIRONE:Dyslipidaemia:0.23), (TEMSIROLIMUS: Dyslipidaemia:0.56), (TERBUTALINE SULFATE:Dyslipidaemia:2.39), (THEOPHYLLINE:Dyslipidaemia:0), (THROMBOMODULIN ALFA:Dyslipidaemia:0.13), (TIARAMIDE:Dyslipidaemia:0.12), (TIGECYCLINE:Dyslipidaemia: 0.48), (TILIDINE:Dyslipidaemia:0.44), (TIMOLOL\TRAVOPROST:Dyslipidaemia:0.97), (TIP EPIDINE HIBENZATE:Dyslipidaemia:0.89), (TIPIRACIL HYDROCHLORIDE\TRIFLURIDINE:Dyslipidaemia:0.31), (TOFISOPAM:Dyslipidaemia:0.42), (TRIMIPRAMINE MALEATE:Dyslipidaemia:1.49), (VILAZODONE HYDROCHLORIDE:Dyslipidaemia:0.32), (VITAMIN A:Dyslipidaemia:1), (XIPAMIDE:Dyslipidaemia:0.97), (YEAST:Dyslipidaemia:0.05), (ZILEUTO N:Dyslipidaemia:0.31), (CHOLESTYRAMINE:Dysmenorrhoea:0.33), (CLONIDINE:Dysmenorrhoea: 0.16), (CLOZAPINE:Dysmenorrhoea:0), (CODEINE:Dysmenorrhoea:0.29), (CORTISONE\HYDROCORTISONE:Dysmenorrhoea:0.16), (DACLIZUMAB:Dysmenorrhoea:0.15), (DILTIA ZEM HYDROCHLORIDE:Dysmenorrhoea:0.13), (DIMENHYDRINATE:Dysmenorrhoea:0.16), (DU LOXETINE:Dysmenorrhoea:0.59), (ENALAPRIL:Dysmenorrhoea:0.77), (ENALAPRIL MALEATE:Dysmenorrhoea:0.08), (EPINEPHRINE:Dysmenorrhoea:0.46), (EVOLOCUMAB: Dysmenorrhoea:0.34), (GLIMEPIRIDE:Dysmenorrhoea:0.02), (HYDROXYCHLOROQUINE DIPHOSPHATE:Dysmenorrhoea:0.06), (HYDROXYZINE PAMOATE:Dysmenorrhoea:0.93), (IBRUTINIB:Dysmenorrhoea:0.45), (ISOPROPYL ALCOHOL\LEUPROLIDE ACETATE\NORETHINDRONE ACETATE:Dysmenorrhoea:0.05), (METOPROLOL TARTRATE:Dysmenorrhoea:0.02), (NORETHINDRONE ACETATE:Dysmenorrhoea:0.1), (NORTRIPTYLINE:Dysmenorrhoea:0.06), (OXCARBAZEPIN E:Dysmenorrhoea:1.02), (OXYGEN:Dysmenorrhoea:0.08), (PAROXETINE\PAROXETINE HYDROCHLORIDE:Dysmenorrhoea:0.48), (PENICILLIN:Dysmenorrhoea:0.14), (PENTOSAN POLYSULFATE SODIUM:Dysmenorrhoea:0.04), (PRAMIPEXOLE DIHYDROCHLORIDE:Dysmenorrhoea:0.2), (PRIMIDONE:Dysmenorrhoea:0.34), (RAMIPRIL:Dysmenorrhoea:0.33), (TAMSULOSIN:Dysmenorrhoea:0.05), (TIZANIDINE:Dysmenorrhoea:00.03), (VERAPAMIL HYDROCHLORIDE:Dysmenorrhoea:0.17), (VILAZODONE HYDROCHLORIDE:Dysmenorrhoea:0.32), (ZIPRASIDONE HYDROCHLORIDE\ZIPRASIDONE MESYLATE:Dysmenorrhoea:0.15), (AMPHETAMINE ASPARTATE\AMPHETAMINE SULFATE\DEXTROAMPHETAMINE SACCHARATE\DEXTROAMPHETAMINE SULFATE:Dyspareunia:0.64),(ARMODAFINIL:Dyspareunia:0.41), (CERTOLIZUMAB PEGOL:Dyspareunia:0.03), (EVOLOCUMAB:Dyspareunia:0.17), (LISDEXAMFETAMINE DIMESYLATE:Dyspareunia:0.18), (PANTOPRAZOLE:Dyspareunia:0.12), (SODIUM OXYBATE:Dyspareunia:0.54), (TERIFLUNOMIDE:Dyspareunia:0.14), (.ALPHA.-TOCOPHEROL\VITAMINS:Dyspepsia:0.1), (.ALPHA.1-PROTEINASE INHIBITOR HUMAN:Dyspepsia:1.79), (ABOBOTULINUMTOXINA:Dyspepsia:1.09), (ACETAMINOPHE N\CAFFEINE\OPIUM:Dyspepsia:1.03), (ACETAMINOPHEN\DICHLORALPHENAZONE\IS OMETHEPTENE:Dyspepsia:0.56), (ACETAMINOPHEN\METHOCARBAMOL:Dyspepsia:0.51), (ALBUTEROL\GUAIFENESIN:Dyspepsia:0.04), (ALBUTEROL\IPRATROPIUM BROMIDE:Dyspepsia:0.23), (ALCOHOL:Dyspepsia:1.68), (ALISKIREN HEMIFUMARATE\HYDROCHLOROTHIAZIDE:Dyspepsia:0.1), (ALOGLIPTIN BENZOATE:Dyspepsia:0.43), (AMILORIDE HYDROCHLORIDE\FUROSEMIDE:Dyspepsia:0.21), (AMINO ACIDS\DEXTROSE\ELECTROLYTES NOS:Dyspepsia:1.82), (AMINOSALICYLIC ACID:Dyspepsia:0.08), (ANAGRELIDE:Dyspepsia:0.23), (ASCORBIC ACID/CHROMIUM/SELENIUM/VANADYL SULFATE:Dyspepsia:0.24), (ASCORBIC ACID\BIOTIN\CALCIUM PANTOTHENATE\CYANOCOBALAMIN\FOLIC ACID\NIACINAMIDE\PYRIDOXINE HYDROCHLORIDE\RIBOFLAVIN\THIAMINE MONONITRATE:Dyspepsia:0.19), (ASENAPINE MALEATE:Dyspepsia:0.11), (ASPIRIN\CLOPIDOGREL:Dyspepsia:0.77), (ATORVASTATIN CALCIUM\EZETIMIBE:Dyspepsia:0.1), (BAMIFYLLINE HYDROCHLORIDE:Dyspepsia:0.6), (BAZEDOXIFENE ACETATE\ESTROGENS, CONJUGATED:Dyspepsia:0.38), (BELATACEPT:Dyspepsia:0.7), (BENSERAZIDE\LEVODO PA:Dyspepsia:0.47), (BERAPROST SODIUM:Dyspepsia:0.77), (BETAMETHASONE DIPROPIONATE\CALCIPOTRIENE:Dyspepsia:1.04), (BETAMETHASONE DIPROPIONATE\CALCIPOTRIENE HYDRATE:Dyspepsia:0.14), (BILASTINE:Dyspepsia:1.18), (BIMATOPROST\TIMOLOL:Dyspepsia: 0.6), (BOSUTINIB:Dyspepsia:0.34), (BOTULINUM TOXIN TYPE A:Dyspepsia:1.17), (BUPIVACAINE HYDROCHLORIDE:Dyspepsia:0.54), (CABOZANTINIB S-MALATE:Dyspepsia:0.75), (CAFFEINE\DIPYRONE\ORPHENADRINE:Dyspepsia:0.51), (CALCIFEDIOL: Dyspepsia:0.05), (CALCIUM PANTOTHENATECYANOCOBALAMINNIACINAMIDEPYRIDOXINE HYDROCHLORIDERIBOFLAVINTHIAMINE MONONITRATE:Dyspepsia:0.86), (CARBAMIDE PEROXIDE:Dyspepsia:0.1), (CARBOXYMETHYLCELLULOSE SODIUMGLYCERINPOLYSORBATE 80:Dyspepsia:0.69), (CARTEOLOL:Dyspepsia:0.18), (CARVEDILOL PHOSPHATE:Dyspepsia:2.91), (CEFPROZIL:Dyspepsia:0.74), (CEFTAZIDIME SODIUM:Dyspepsia:0.31), (CEFUROXIME SODIUM:Dyspepsia:0.01), (CELIPROLOL:Dyspepsia:0.48), (CERIVASTATIN SODIUM:Dyspepsia:0.59), (CICLOPIROX OLAMINE:Dyspepsia:0.24), (CILNIDIPINE:Dyspepsia:0.11), (CLADRIBINE:Dyspepsia:0.08), (CLOFARABINE:Dyspepsia:0.18), (CLOXACILLIN SODIUM:Dyspepsia:0.13), (COBICISTATDARUNAVIR ETHANOLATE:Dyspepsia:0.59), (COLCHICINEDICYCLOMINE HYDROCHLORIDE:Dyspepsia:0.2), (CORTISONE ACETATE:Dyspepsia:0.71), (CYAMEMAZINE:Dyspepsia:0.28), (CYCLOPENTOLATE:Dyspepsia: 0.11), (CYPROHEPTADINE HYDROCHLORIDE:Dyspepsia:1.35), (CYPROTERONE:Dyspepsia:0.05), (DACLIZUMAB:Dyspepsia: 2.65), (DANTROLENE SODIUM:Dyspepsia:1.01), (DARUNAVIR:Dyspepsia:0.91), (DARUNAVIRRITONAVIR:Dyspepsia: 0.34), (DEGARELIX:Dyspepsia:0.76), (DEGARELIX ACETATE:Dyspepsia:0.7), (DEOXYCHOLIC ACID:Dyspepsia:0.1), (DEXAMETHASONE ACETATE:Dyspepsia:1.83), (DEXMETHYLPHENIDATE HYDROCHLORIDE:Dyspepsia:0.25), (DIENOGEST:Dyspepsia:0.04), (DIETARY SUPPLEMENTHERBALSMILK THISTLE:Dyspepsia:0.51), (DIETARY SUPPLEMENTRESVERATROL:Dyspepsia:0), (DIFLUNISAL:Dyspepsia:0.65), (DIPYRONE PROMETHAZINE HYDROCHLORIDE:Dyspepsia:0.83), (DISOPYRAMIDE:Dyspepsia:0.06), (DOCUSATE SODIUMSENNOSIDES A AND B:Dyspepsia:0.32), (DOPAMINE HYDROCHLORIDE:Dyspepsia:0.46), (DORZOLAMIDE HYDROCHLORIDE:Dyspepsia:1.74), (DOXERCALCIFEROL:Dyspepsia:0.01), (DROXIDOPA:Dyspepsia:0.6), (ECONAZOLE:Dyspepsia:2.68), (EDOXABAN TOSILATE MONOHYDRATE:Dyspepsia:0.22), (ELBASVIRGRAZOPREVIR:Dyspepsia:0.79), (ELOTUZ UMAB:Dyspepsia:1.86), (EMPAGLIFLOZINLINAGLIPTIN:Dyspepsia:1.92), (EMTRICITABI NERILPIVIRINE HYDROCHLORIDETENOFOVIR DISOPROXIL FUMARATE:Dyspepsia:0.89), (EMTRICITABINERILPIVIRINETENOFOVIR DISOPROXIL FUMARATE:Dyspepsia:0.44), (ENALAPRIL MALEATEHYDROCHLOROTHIAZIDE:Dyspepsia:1.12), (ENALAPRIL MALEATELERCANIDIPINE HYDROCHLORIDE:Dyspepsia:0.9), (EPINASTINE HYDROCHLORIDE:Dyspepsia:1.19), (ESMOLOL HYDROCHLORIDE:Dyspepsia:0.27), (ETHACRYNIC ACID:Dyspepsia:0.96), (ETHINYL ESTRADIOLGESTODENE:Dyspepsia:0.58), (ETHINYL ESTRADIOLNORGESTREL:Dyspepsia:0.99), (ETHIODIZED OIL:Dyspepsia:0.03), (ETOPOSIDE PHOSPHATE:Dyspepsia:0.76), (ETRETINATE:Dyspepsia:1.07), (EUCALYPTOLMENTHOL METHYL SALICYLATETHYMOL:Dyspepsia:0.13), (FENOTEROL HYDROBROMIDE:Dyspepsia:1.66), (FENOTEROL HYDROBROMIDEIPRATROPIUM BROMIDE:Dyspepsia:1.27), (FERROUS BISGLYCINATE:Dyspepsia:0.61), (FERROUS SULFATEFOLIC ACID:Dyspepsia:1.1), (FERROUS SULFATEGLYCINE:Dyspepsia:1.19), (FLUCYTOSINE:Dyspepsia:0.21), (FLUOROURACIL LEUCOVORIN CALCIUMOXALIPLATIN:Dyspepsia:0.29), (FLUOXETINE HYDROCHLORIDEOLANZAPINE:Dyspepsia:0.21), (FORMOTEROL FUMARATEMOMETASONE FUROATE:Dyspepsia:1.79), (FOSPHENYTOINFOSPHENYTOIN SODIUM:Dyspepsia:0.44), (FROVATRIPTAN SUCCINATE:Dyspepsia:0.5), (GIMERACILOTERACILTEGAFUR:Dyspepsia:1.42), (GLIME PIRIDEROSIGLITAZONE MALEATE:Dyspepsia:0.07), (GLYCOPYRROLATEINDACATEROL MALEATE:Dyspepsia:1.05), (GOLD:Dyspepsia:0.2), (GUANFACINE:Dyspepsia:1.23), (HALCI NONIDE:Dyspepsia:0.02), (HALOPERIDOL DECANOATE:Dyspepsia:0.38), (HIGH DENSITY POLYETHYLENE:Dyspepsia:0.07), (HORSE CHESTNUT:Dyspepsia:0.31), (HYDROCHLOROTHIAZIDEMETOPROLOL SUCCINATE:Dyspepsia:0.33), (HYDROCHLOROTHIAZIDEQUINAPRIL HYDROCHLORIDE:Dyspepsia:0.53), (HYDROCHLOROTHIAZIDERAMIPRIL:Dyspepsia:0.48), (IDARUBICIN:Dyspepsia:0.64), (ILOPROST:Dyspepsia:2.16), (IMIPENEM:Dyspepsia:1.15), (INTERFERON ALFA-2A OR INTERFERON ALFA-2B:Dyspepsia:0.53), (INTERFERON GAMMA-1B:Dyspepsia:1.36), (IRON POLYMALTOSE:Dyspepsia:0.35), (ISONIAZIDPYRAZINAMIDERIFAMPIN:Dyspepsia:0.05), (ISONIAZIDRIFAMPIN:Dyspepsia:0.69), (KETAMINE HYDROCHLORIDE:Dyspepsia:0.05), (LANREOTIDE:Dyspepsia:1.04), (LAPATINIB:Dyspepsia: 0.51), (LATANOPROSTTIMOLOL:Dyspepsia:0.37), (LEVOLEUCOVORIN CALCIUM:Dyspepsia:0.84), (LINSIDOMINE:Dyspepsia:0.36), (LORNOXICAM:Dyspepsia:0.29), (LUMACAFTOR:Dyspepsia:0.08), (LUTETIUM LU-177:Dyspepsia:0.81), (MAGNESIUM ASPARTATEPOTASSIUM ASPARTATE:Dyspepsia:0.45), (MAGNESIUM PIDOLATE:Dyspepsia:0.08), (MECLIZINE MONOHYDROCHLORIDENIACIN:Dyspepsia:0.11), (MELPERONE:Dyspepsia:0.26), (METF ORMIN HYDROCHLORIDESAXAGLIPTIN:Dyspepsia:0.48), (METFORMIN PAMOATE:Dyspepsia:0.7), (METFORMINSITAGLIPTIN:Dyspepsia:0.32), (METHENAMINE MANDELATE:Dyspepsia:0.41), (METILDIGOXIN:Dyspepsia:0.19), (MEXILETINE:Dyspepsia: 0.62), (MIGLUSTAT:Dyspepsia:0.49), (MOEXIPRIL HYDROCHLORIDE:Dyspepsia:1.46), (NALOXONE:Dyspepsia:0.9), (NAPROXEN SODIUMSUMATRIPTAN SUCCINATE:Dyspepsia:0.23), (NEFAZODONE:Dyspepsia:0.36), (NEFAZODONE HYDROCHLORIDE:Dyspepsia:0.23), (NEOMYCIN:Dyspepsia:0.96), (NEOSTIGMINE:Dyspepsia: 0.1), (OBETICHOLIC ACID:Dyspepsia:0.02), (OPIPRAMOL:Dyspepsia:0.91), (ORPHENADRINE:Dyspepsia:1.27), (O XYBUTYNINOXYBUTYNIN CHLORIDE:Dyspepsia:0.97), (PARATHYROID HORMONE:Dyspepsia:0.65), (PENICILLAMINE:Dyspepsia:0.23), (PENTOSTATIN:Dyspepsia: 1.31), (PIMOBENDAN:Dyspepsia:0.2), (PIRITRAMIDE:Dyspepsia:0.27), (POLYMYXIN B SULFATETRIMETHOPRIM SULFATE:Dyspepsia:0.03), (PORFIMER SODIUM:Dyspepsia:0.49), (PRAMOXINE HYDROCHLORIDE:Dyspepsia:0.29), (PREDNISOLONE ACETATESULFACETAMIDE SODIUM:Dyspepsia:0.03), (PREDNISOLONE SODIUM PHOSPHATE:Dyspepsia:1.07), (PRISTINAMYCIN:Dyspepsia:1.1), (PROTRIPTYLINE HYDROCHLORIDE:Dyspepsia:0.99), (PYRIMETHAMINE:Dyspepsia:0.52), (RADIATION THERAPY:Dyspepsia:2.16), (REGORAFENIB:Dyspepsia:2.36), (RILMENIDINE:Dyspepsia:0.16), (RISEDRONIC ACID:Dyspepsia:0.05), (RUPATADINE FUMARATE:Dyspepsia:0.25), (SARPOGRELATE:Dyspepsia:0.44), (SELEGILINE:Dyspepsia: 0.71), (SIBUTRAMINE HYDROCHLORIDE:Dyspepsia:0.66), (SILICON DIOXIDE:Dyspepsia:0.48), (SODIUM PHOSPHATE, DIBASIC, ANHYDROUSSODIUM PHOSPHATE, DIBASIC, HEPTAHYDRATESODIUM PHOSPHATE, MONOBASIC, MONOHYDRATE:Dyspepsia:0.2), (SOMATOSTATIN:Dyspepsia:0.03), (ST. JOHN'S WORT:Dyspepsia:0), (SUFENTANIL CITRATE:Dyspepsia:0.65), (SULFACETAMIDE SODIUMSULFUR:Dyspepsia:0.36), (TAFAMIDIS MEGLUMINE:Dyspepsia:0.08), (TAFLUPROST:Dyspepsia:1.01), (TASIMELTEON:Dyspepsia: 0.35), (TEDUGLUTIDE:Dyspepsia:0.57), (TERCONAZOLE:Dyspepsia:0.31), (TETRABENAZI NE:Dyspepsia:0.28), (TETRAZEPAM:Dyspepsia:0.12), (TRIMIPRAMINE MALEATE:Dyspepsia:1.28), (TRIPTORELIN PAMOATE:Dyspepsia:1.04), (URAPIDIL:Dyspepsia:1.14), (VELAGLUCERASE ALFA:Dyspepsia:0.24), (VOGLIBOSE:Dyspepsia:0.4), (WARFARIN POTASSIUM:Dyspepsia:0.59), (YEAST:Dyspepsia:1.43), (ZEAXANTHIN:Dyspepsia:0), (ZILE UTON:Dyspepsia:1.12), (ZIV-AFLIBERCEPT:Dyspepsia:0.55), (BENAZEPRIL HYDROCHLORIDE:Dysphagia:0.04), (BUPRENORPHINE HYDROCHLORIDE:Dysphagia:0.31), (BUSPIRONE HYDROCHLORIDE:Dysphagia:0.65), (CALCIUM CITRATE:Dysphagia:0.1), (CANDESARTAN:Dysphagia:0.38), (CANDESARTAN CILEXETILHYDROCHLOROTHIAZIDE:Dysphagia:0.11), (CARFILZOMIB:Dysphagia:0.17), (CARVEDILOL PHOSPHATE:Dysphagia:0.01), (CERTOLIZUMAB PEGOL:Dysphagia:0.9), (CLINDAMYCINCLINDAMYCIN PHOSPHATE:Dysphagia:0.95), (DASATINIB:Dysphagia:0.36), (DEVICE:Dysphagia:0.04), (DI CLOFENAC:Dysphagia:0.61), (DIVALPROEX SODIUM:Dysphagia:0.53), (DULOXETINE:Dysphagia:0.51), (DUTASTERIDE:Dysphagia:0.18), (ENALAPRIL:Dysphagia:1.6), (EVOLOCUMAB:Dysphagia:0.56), (FERROUS SULFATEFERROUS SULFATE, DRIED:Dysphagia:1.08), (FLUTICASONE:Dysphagia:0.11), (HYDROCHLOROTHIAZIDEVA LSARTAN:Dysphagia:0.09), (HYDROXYZINE PAMOATE:Dysphagia:0.49), (ILOPROST:Dysphagia:0.13), (INDOMETHACIN:Dysphagia:0.11), (LEVALBUTEROL HYDROCHLORIDE:Dysphagia:0.32), (LINAGLIPTIN:Dysphagia:0.32), (LITHIUM CARBONATE:Dysphagia:0.21), (METAXALONE:Dysphagia:0.35), (METHADONE HYDROCHLORIDE:Dysphagia:0.15), (METRONIDAZOLEMETRONIDAZOLE HYDROCHLORIDE:Dysphagia:0.45), (NAPROXEN SODIUM:Dysphagia:0.14), (NEBIVOLOL:Dysphagia:0.23), (NILOTINIB:Dysphagia:0.34), (NO RTRIPTYLINE:Dysphagia:0.06), (OMEGA-3-ACID ETHYL ESTERS:Dysphagia:0.08), (OXCARBAZEPINE:Dysphagia:0.57), (PAROXETINEPAROXETI NE HYDROCHLORIDE:Dysphagia:0.09), (PENICILLIN:Dysphagia:0.12), (PRAMIPEXOLE DIHYDROCHLORIDE:Dysphagia:0.51), (QUETIAPINE:Dysphagia:0.41), (RISEDRONATE SODIUM:Dysphagia:0.18), (RIZATRIPTAN BENZOATE:Dysphagia:0.03), (SACUBITRILVALSARTAN:Dysphagia:0.52), (SUMATRIPTA N:Dysphagia:0.03), (SUMATRIPTANSUMATRIPTAN SUCCINATE:Dysphagia:0.13), (TERIFLUNOMIDE:Dysphagia:0.78), (THALIDOMIDE:Dysphagia: 0.22), (UMECLIDINIUM BROMIDEVILANTEROL TRIFENATATE:Dysphagia:0.28), (ZOLPIDEMZOLPIDEM TARTRATE:Dysphagia:0.84), (ACECLOFENAC:Dyspnoea:0.15), (ACETAMINOPHENCAFF EINE:Dyspnoea:0.79), (ACETAMINOPHENCAFFEINEOPIUM:Dyspnoea:0.54), (ACETAMI NOPHENTRAMADOL:Dyspnoea:1.24), (AGALSIDASE BETA:Dyspnoea:2.29), (AJMALINE:Dyspnoea:0.21), (ALBUTEROLGUAIFENESIN:Dyspnoea: 0.55), (ALGLUCOSIDASE ALFA:Dyspnoea:1.71), (ALUMINUM HYDROXIDEMAGNESIUM HYDROXIDESIMETHICONE:Dyspnoea:0.28), (AMINO ACIDSDEXTROSEELECTROLYTES NOS:Dyspnoea:1.27), (AMLODIPINE BESYLATEATORVASTATIN CALCIUM:Dyspnoea:1.11), (AMLODIPINEPERINDOPRIL:Dyspnoea:1), (AMOXICILLINC LARITHROMYCINESOMEPRAZOLE:Dyspnoea:0.56), (ANAGRELIDE HYDROCHLORIDE:Dyspnoea:0.74), (ANAKINRA:Dyspnoea:0.18), (ARIPIPRAZOLE LAUROXIL:Dyspnoea:0.24), (ASCORBIC ACID/CHROMIUM/SELENIUM/VANADYL SULFATE:Dyspnoea:0.14), (ASCORBIC ACIDBIOTINCALCIUM PANTOTHENATECYANOCOBALAMINFOLIC ACIDNIACINAMIDEPYRIDOXINE HYDROCHLORIDERIBOFLAVINTHIAMINE MONONITRATE:Dyspnoea:0.2), (ASPIRINMAGNESIUM OXIDE:Dyspnoea:0.55), (AVANAFIL:Dyspnoea:0.11), (AZELNIDIPINE:Dyspnoea:0.15), (AZI THROMYCIN OR METHOTREXATE SODIUM:Dyspnoea:0.04), (BALSALAZIDE DISODIUM:Dyspnoea:0.83), (BECLOMETHASONE DIPROPIONATEFORMOTEROL:Dyspnoea:0.29), (BELATACEPT:Dyspnoea:0.42), (BERAP ROST:Dyspnoea:0.41), (BIFIDOBACTERIUM SPP.:Dyspnoea:0.26), (BILASTINE:Dyspnoea:0.81), (BIMATOPROSTTIMOLOL:Dyspnoea:0.11), (BOCOCIZUMAB:Dyspnoea:0.22), (BOSUTINIB:Dyspnoea:0.04), (BRINZOLAMIDETIM OLOL MALEATE:Dyspnoea:0.3), (BUPIVACAINE HYDROCHLORIDE:Dyspnoea:0.52), (BUPRENORPHINENALOXONE:Dyspnoea:0.28), (CA LCIUM CARBONATEERGOCALCIFEROL:Dyspnoea:0.24), (CALCIUM CARBONATEERGOCALCIFEROLRETINOL:Dyspnoea:1.2), (CALCIUM PANTOTHENATECYANOCOBALAMINNIACINAMIDEPYRIDOXINE HYDROCHLORIDERIBOFLAVINTHIAMINE MONONITRATE:Dyspnoea:0.17), (CANAGLIFLOZINMETFORMIN HYDROCHLORIDE:Dyspnoea:1.89), (CARBAMIDE PEROXIDE:Dyspnoea:0.07), (CARBIDOPAENTACAPONELEVODOPA:Dyspnoea:0.11), (C ARTEOLOL:Dyspnoea:0.03), (CEFOXITINCEFOXITIN SODIUM:Dyspnoea:0.4), (CEFPROZIL:Dyspnoea:0.55), (CEFTAZIDIME SODIUM:Dyspnoea:0.1), (CELIPROLOL:Dyspnoea:0.17), (CERIVASTATIN SODIUM:Dyspnoea:0.23), (CERTOPARIN SODIUM:Dyspnoea:0.03), (CEVIMELINE HYDROCHLORIDE:Dyspnoea:0.54), (CHLORDIAZEPOXIDE:Dyspnoea:0.14), (CHONDROI TIN SULFATE (CHICKEN):Dyspnoea:0.46), (CIPROFIBRATE:Dyspnoea:1.02), (CLIKSTAR:Dyspnoea:0.29), (CLOMIPRAMINE HYDROCHLORIDE:Dyspnoea:1.21), (CLOXACILLIN SODIUM:Dyspnoea:0.15), (CLOXAZOLAM:Dyspnoea:0.19), (DABRAFENIB MESYLATE:Dyspnoea:0.92), (DACLIZUMAB:Dyspnoea:2.24), (DEGARELIX ACETATE:Dyspnoea:0.06), (DEXAMETHASONE ACETATE:Dyspnoea:1.07), (DEXMEDETOMIDINE HYDROCHLORIDE:Dyspnoea:0.53), (DICLOXACILLIN:Dyspnoea:0.39), (DIFLUNISAL:Dyspnoea: 0.23), (DIMETHYL SULFOXIDE:Dyspnoea:0.18), (DISOPYRAMIDE:Dyspnoea:0.02), (DOBUTAMINEDOBUTA MINE HYDROCHLORIDE:Dyspnoea:0.88), (DOLASETRON MESYLATE:Dyspnoea:1.06), (DOLUTEGRAVIR SODIUM:Dyspnoea:0.08), (ECALLANTIDE:Dyspnoea:0.81), (EFAVIRENZEMTRICITABINE TENOFOVIR DISOPROXIL FUMARATE:Dyspnoea:0.16), (EFINACONAZOLE:Dyspnoea:0.13), (ELBASVIRGRAZOPRE VIR:Dyspnoea:0.64), (ELTROMBOPAG OLAMINE:Dyspnoea:1.03), (ENALAPRIL MALEATEHYDROCHLOROTHIAZIDE:Dyspnoea:0.55), (EPIRUBICIN HYDROCHLORIDE:Dyspnoea:0.06), (ERIBULIN:Dyspnoea:0.78), (ERTAPENEM SODIUM:Dyspnoea:0.79), (ESMOLOL HYDROCHLORIDE:Dyspnoea:0.58), (ESTRIOL:Dyspnoea:0.73), (ESTROGENS, ESTERIFIEDMETHYLTESTOSTERONE:Dyspnoea:0.24), (ETHACRYNIC ACID:Dyspnoea:0.83), (ETHINYL ESTRADIOLNORETHINDRONE:Dyspnoea:0.25), (ETHINYL ESTRADIOLNORGESTREL:Dyspnoea:0.53), (ETOPOSIDE PHOSPHATE:Dyspnoea:0.09), (ETRETINATE:Dyspnoea:0.55), (EUCALYPTOLMENTHOL METHYL SALICYLATETHYMOL:Dyspnoea:0.12), (FERROUS BISGLYCINATE:Dyspnoea:0.23), (FLUNARIZINE HYDROCHLORIDE:Dyspnoea:0.22), (FLURAZEPAMFLURAZEPAM HYDROCHLORIDE:Dyspnoea:0.56), (FLURBIPROFEN:Dyspnoea:0.34), (FOSFOMYCIN:Dyspnoea: 0.45), (FOSINOPRIL SODIUM:Dyspnoea:0.22), (FOSPHENYTOINFOSPHENYTOIN SODIUM:Dyspnoea:0.31), (GEFITINIB:Dyspnoea:0.58), (GIMERACILOTERACILTEGAFUR:Dyspnoea:1.21), (GLUTAMINE:Dyspnoea:0.45), (GUANFACINE:Dyspnoea:1.39), (HALOPER IDOL DECANOATE:Dyspnoea:0.01), (HYALURONATE SODIUM:Dyspnoea:1.08), (HYDROXYPROPYL CELLULOSE:Dyspnoea:0.02), (HYOSCYAMINE SULFATEMETHENAMINEMETHYLENE BLUEPHENYL SALICYLATESODIUM PHOSPHATE, MONOBASIC, MONOHYDRATE:Dyspnoea:0.3), (IDARUCIZUMAB:Dyspnoea:0.22), (IMIGLUCERASE:Dyspnoea: 0.98), (IMIPENEM:Dyspnoea:1.06), (IMIQUIMOD:Dyspnoea:0.35), (IMMUNE GLOBULIN NOS:Dyspnoea:0.49), (INDAPAMIDEPERINDOPRIL:Dyspnoea:2.5), (INFLIXIMAB-DYYB:Dyspnoea:0.86), (INTERFERON ALFA-2A OR INTERFERON ALFA-2B:Dyspnoea:0.16), (INTERFERON ALFA-2B:Dyspnoea:1.93), (IRONVITAMINS:Dyspnoea:0.31), (IVERMECTIN:Dyspnoea:0.28), (KET AMINE HYDROCHLORIDE:Dyspnoea:0.02), (LANREOTIDE:Dyspnoea:0.61), (LAPATINIB DITOSYLATE:Dyspnoea:1.18), (LATANOPROSTTIMOLOL:Dyspnoea:0.01), (LEVOMEFOL ATE CALCIUM:Dyspnoea:0.78), (LEVOTHYROXINE SODIUMPOTASSIUM IODIDE:Dyspnoea:0.36), (LINSIDOMINE:Dyspnoea:0.3), (LOMUSTINE:Dyspnoea:0.2), (LUM ACAFTOR:Dyspnoea:1.15), (LUTETIUM LU-177:Dyspnoea:0.39), (MEDROXYPROGESTERONE:Dyspnoea:0.8), (MELPERONE:Dyspnoea: 0.08), (METFORMIN HYDROCHLORIDEROSIGLITAZONE MALEATE:Dyspnoea:0.34), (METFORMIN HYDROCHLORIDESAXAGLIPTIN:Dyspnoea:0.06), (METFORMIN HYDROCHLORIDESAXAGLIPTIN HYDROCHLORIDE:Dyspnoea:0.59), (METFORMIN PAMOATE:Dyspnoea:0.2), (METHENAMINE MANDELATE:Dyspnoea:0.14), (METHENOLONE ENANTHATE:Dyspnoea:0.16), (MIGLUSTAT:Dyspnoea:0.5), (NEOMYCIN:Dyspnoea:0.46), (NEOSTIGMINE:Dyspnoea:0.11), (NOREPINEPHRINE:Dyspnoea:1.04), (OPIPRAMOL:Dyspnoea: 0.45), (OXYBUTYNINOXYBUTYNIN CHLORIDE:Dyspnoea:0.55), (OXYMORPHONE:Dyspnoea:0.28), (PARATHYROID HORMONE:Dyspnoea:0.45), (PEGINTERFERON ALFA-2A OR PEGINTERFERON ALFA-2B OR PEGINTERFERON BETA-1A OR PEGINTERFERON LAMBDA-1A:Dyspnoea:0.09), (PEGINTERFERON ALFA-2B:Dyspnoea:0.75), (PENTAMIDINE ISETHIONATE:Dyspnoea:0.77), (PENTOSTATIN:Dyspnoea:0.78), (PERPHENAZINE:Dyspnoea: 0.15), (PIRBUTEROL ACETATE:Dyspnoea:0.07), (POLYETHYLENE GLYCOL 400:Dyspnoea:0.22), (POLYETHYLENE GLYCOL 4000:Dyspnoea:1.28), (PORFIMER SODIUM:Dyspnoea:0.26), (PROTRIPTYLINE HYDROCHLORIDE:Dyspnoea:0.83), (PRUCALOPRIDE SUCCINATE:Dyspnoea:0.26), (PYRIMETHAMINE:Dyspnoea:0.12), (QUINACRINE:Dyspnoea: 0.81), (RADIUM RA-223 DICHLORIDE:Dyspnoea:0.31), (RALTEGRAVIR POTASSIUM:Dyspnoea:1.39), (REMIFENTANIL:Dyspnoea:0.07), (RESLIZUMAB:Dyspnoea:0.09), (RILMENIDINE PHOSPHATE:Dyspnoea:0.9), (RILPIVIRINE HYDROCHLORIDE:Dyspnoea:0.1), (SELEGILINE HYDROCHLORIDE:Dyspnoea:0.31), (SEVELAMER HYDROCHLORIDE:Dyspnoea:0.86), (SIBUTRAMINE HYDROCHLORIDE:Dyspnoea:0.37), (SILICON DIOXIDE:Dyspnoea:0.15), (SODIUM PHOSPHATE, DIBASIC, ANHYDROUSSODIUM PHOSPHATE, DIBASIC, HEPTAHYDRATESODIUM PHOSPHATE, MONOBASIC, MONOHYDRATE:Dyspnoea:0.3), (SOLIFENACIN:Dyspnoea:0.2), (STREPTOMYCINSTREP TOMYCIN SULFATE:Dyspnoea:0.48), (TEDUGLUTIDE:Dyspnoea:0.15), (TELAPREVIR:Dyspnoea:0.05), (TEMOZOLOMIDE:Dyspnoea:0.58), (TEMSIROLIMUS:Dyspnoea:0.53), (TESTOSTERONE UNDECANOATE:Dyspnoea:1.22), (TETRACYCLINE:Dyspnoea:1.93), (TIMOLOL MALEATETRAVOPROST:Dyspnoea:0.24), (TRABECTEDIN:Dyspnoea:0.26), (TRICHLORM ETHIAZIDE:Dyspnoea:0.19), (TRIMIPRAMINE MALEATE:Dyspnoea:0.64), (TRIPTORELIN PAMOATE:Dyspnoea:0.58), (URAPIDIL:Dyspnoea:1.03), (VALDECOXIB:Dyspnoea:0.04), (ZI CONOTIDE ACETATE:Dyspnoea:0.53), (ZIV-AFLIBERCEPT:Dyspnoea:0.16), (ALCOHOL:Dystonia:0.07), (AMIODARONE:Dystonia:0.05), (DACLIZUMAB:Dystonia:0.04), (DEXLANSOPRAZOLE:Dystonia:0.73), (DILTIAZEM HYDROCHLORIDE:Dystonia:0.26), (DULOXETINE:Dystonia:0.2), (ENALAPRIL:Dystonia:0.17), (EPINEPHRINE:Dystonia:0.23), (ESZOPICLONE:Dystonia:0.12), (FERROUS SULFATEFERROUS SULFATE, DRIED:Dystonia:0.18), (GUANFACINE:Dystonia:0.01), (HYDROXYZINE PAMOATE:Dystonia:0.46), (LISDEXAMFETAMINE DIMESYLATE:Dystonia:0.49), (PANTOPRAZOLE:Dystonia:0.24), (PAROXETINE:Dystonia:00.56), (PRAVASTATIN SODIUM:Dystonia:0.13), (PRIMIDONE:Dystonia:0.29), (SACUBITRILVALSARTAN:Dystonia: 0.14), (TERIFLUNOMIDE:Dystonia:0.4), (VARENICLINE TARTRATE:Dystonia:1.24), (VITAMIN B:Dystonia:0.02), (WARFARIN:Dystonia:0.16), (WARFARIN SODIUM:Dystonia:0.07), (ZOLPIDEMZOLPIDEM TARTRATE:Dystonia:0.29), (ACARBOSE:Dysuria:0.66), (ACETAMINOPHENBUTALBITAL CAFFEINE:Dysuria:0.84), (ACETAZOLAMIDE:Dysuria:0.21), (ADO-TRASTUZUMAB EMTANSINE:Dysuria:0.71), (AGALSIDASE BETA:Dysuria:0.1), (ALBIGLUTIDE:Dysuria:0.08), (ALEMTUZUMAB:Dysuria:0.1), (ALEND RONIC ACID:Dysuria:0.51), (ALUMINUM HYDROXIDEDIMETHICONEMAGNESIUM HYDROXIDE:Dysuria:0.03), (AMISULPRIDE:Dysuria:0.65), (AMLODIPINE BESYLATEATORVASTATIN CALCIUM:Dysuria:0.09), (ANASTROZOLE:Dysuria:1.67),(ARMODAFINIL:Dysuria:0.86), (A TOVAQUONE:Dysuria:0.05), (AXITINIB:Dysuria:0.19), (BENAZEPRILBENAZEPRIL HYDROCHLORIDE:Dysuria:0.16), (BRINZOLAMIDE:Dysuria:0.19), (BUPRENORPHINE HYDROCHLORIDENALOXONE HYDROCHLORIDE:Dysuria:0.64), (CALCIUM ACETATE:Dysuria:0.98), (CALCIUM POLYCARBOPHIL:Dysuria:0.03), (CANAGLIFLOZINMETFORMIN HYDROCHLORIDE:Dysuria:0.71), (CANDESARTAN:Dysuria:1.9), (CANDESARTAN CILEXETILHYDROCHLOROTHIAZIDE:Dysuria:1.02), (CAPTOPRIL:Dysuria:0.67), (CARV EDILOL PHOSPHATE:Dysuria:0.6), (CERITINIB:Dysuria:0.02), (CILASTATIN SODIUMIMIPENEM:Dysuria:0.45), (CINACALCET HYDROCHLORIDE:Dysuria:0.06), (CLINDAMYCINCLINDAMYCIN PHOSPHATE:Dysuria:1.19), (CLONIDINE HYDROCHLORIDE:Dysuria:0.22), (CLOTRIMAZOLE:Dysuria:0.89), (CORTICOTROPIN:Dysuria: 0.15), (CORTISONEHYDROCORTISONE:Dysuria:1.09), (CRIZOTINIB:Dysuria:0.53), ( CYCLOBENZAPRINE:Dysuria:0.67), (DABIGATRAN:Dysuria:0.13), (DABRAFENIB:Dysuria: 0.07), (DACLIZUMAB:Dysuria:0.6), (DANAZOL:Dysuria:0.32), (DASATINIB:Dysuria:1.03), (DOXEPIN HYDROCHLORIDE:Dysuria:0.17), (DRONABINOL:Dysuria:0.56), (ECONAZOLE:Dysuria:0.11), (ELETRIPTAN HYDROBROMIDE:Dysuria:0.61), (ELOTUZUMAB:Dysuria:0.13), (EMPAGLIFLOZINLINA GLIPTIN:Dysuria:0.2), (EPINEPHRINE:Dysuria:0.29), (ESTROGENS, CONJUGATEDMEDROXYPROGESTERONE ACETATE:Dysuria:0.19), (ETORICOXIB:Dysuria:0.47), (EVOLOCUMAB:Dysuria:1.02), (EXE MESTANE:Dysuria:0.29), (FERROUS FUMARATE:Dysuria:0.12), (FISH OIL\OMEGA-3 FATTY ACIDS:Dysuria:0.11), (FLUDROCORTISONE ACETATE:Dysuria:0.41), (FLUTICASONE FUROATE:Dysuria:0.43), (FLUVOXAMINE MALEATE:Dysuria:0.27), (FORMOTEROL:Dysuria:0.02), (FULVESTRANT:Dysuria:0.59), (G EMCITABINE HYDROCHLORIDE:Dysuria:0.22), (GIMERACILOTERACILTEGAFUR:Dysuria:0.67), (GL YCOPYRRONIUMINDACATEROL:Dysuria:0.53), (GUANFACINE:Dysuria:0.01), (HERBAL STURMERIC:Dysuria:0.15), (HYDROXYCHLOROQUINE DIPHOSPHATE:Dysuria:0.02), (ILOPROST:Dysuria:0.27), (IMATINIB MESYLATE:Dysuria:0.39), (IMIPRAMINE HYDROCHLORIDE:Dysuria:0.38), (INDAPAMIDEPERINDOPRIL:Dysuria:1.14), (INTERFE RON ALFA-2A:Dysuria:0.06), (IRINOTECAN HYDROCHLORIDE:Dysuria:1.13), (IRON SUCROSE:Dysuria:0.24), (LABETALOLLABETALOL HYDROCHLORIDE:Dysuria:0.69), (LAPATINIB DITOSYLATE:Dysuria:0.34), (LEUCOVORIN CALCIUM:Dysuria:1.14), (LEVALBUTEROL HYDROCHLORIDE:Dysuria:0.73), (LITHIUM:Dysuria:0.4), (LOMITAPIDE MESYLATE:Dysuria:0.01), (METAMIZOLE:Dysuria:0), (METHENAMINE HIPPURATE:Dysuria:0.44), (METHOCARBAMOL:Dysuria:0.47), (METRONIDAZOLE:Dysuria: 2.17), (NABUMETONE:Dysuria:0.16), (NALOXONE HYDROCHLORIDEOXYCODONE HYDROCHLORIDE:Dysuria:0.73), (NALTREXONE:Dysuria:0.22), (NATEGLINIDE:Dysuria: 0.18), (NILOTINIB:Dysuria:2.04), (NINTEDANIB:Dysuria:0.46), (NORETHINDRONE:Dysuria: 0.09), (PANTOPRAZOLE MAGNESIUM:Dysuria:0.86), (PEGINTERFERON ALFA-2B:Dysuria:0.28), (PEGINTERFERON BETA-1A:Dysuria:0.23), (PEMETREXED DISODIUM:Dysuria:0.59), (PHYTONADIONE:Dysuria:0.12), (PILSICAINIDE HYDROCHLORIDE:Dysuria:0.3), (PIROXICAM:Dysuria:0.18), (PONATINIB:Dysuria:0.1), (P RAZOSIN:Dysuria:0.37), (PRAZOSIN HYDROCHLORIDE:Dysuria:0.01), (PYRAZINAMIDE:Dysuria:0.71), (RABEPRAZOLE:Dysuria: 0.8), (RISEDRONATE SODIUM:Dysuria:0.7), (RIZATRIPTAN BENZOATE:Dysuria:0.65), (ROPINIROLE HYDROCHLORIDE:Dysuria:0.77), (SACUBITRILVALSARTAN:Dysuria:0.97), (SCOPOLA MINE:Dysuria:0.91), (SULFAMETHOXAZOLE:Dysuria:0.63), (TERBINAFINE HYDROCHLORIDE:Dysuria:0.35), (TERBUTALINE SULFATE:Dysuria:0.22), (THALIDOMIDE:Dysuria:0.86), (TINZAPARIN:Dysuria:0.01), (TIOT ROPIUM:Dysuria:0.56), (TRAMETINIB:Dysuria:0.16), (TRIAMTERENE:Dysuria:0.11), (UME CLIDINIUM BROMIDEVILANTEROL TRIFENATATE:Dysuria:0.56), (VILAZODONE HYDROCHLORIDE:Dysuria:0.24), (WARFARIN POTASSIUM:Dysuria:0.04), (ZONISAMIDE:Dysuria:0.02), (BUSPIRONE HYDROCHLORIDE:Ear disorder:0.02), (CALCIUM CARBONATECHOLECALCIFEROL:Ear disorder:0.1), (DICLOFENAC:Ear disorder:0.05), (DILTIAZEM HYDROCHLORIDE:Ear disorder:0.42), (DIPHENHYDRAMINE:Ear disorder:0.16), (DOCUSATE SODIUM:Ear disorder:0.13), (EVOLOCUMAB:Ear disorder:0.3), (IBRUTINIB:Ear disorder:0.41), (METRONIDAZOLE:Ear disorder:0.1), (TERIFLUNOMIDE:Ear disorder:0.09), (TOCILIZUMAB:Ear disorder:0.07), (TOFACITINIB CITRATE:Ear disorder:0.04), (AMIODARONE HYDROCHLORIDE:Ear infection:0.36), (AMPICILLIN:Ear infection:0), (BISOPROLOL FUMARATE:Ear infection:0.66), (BUMETANIDE:Ear infection:0.49), (CALCIUM CARBONATECHOLECALCIFEROL:Ear infection:0.92), (CALCIUM CITRATE:Ear infection:0.12), (CANDESARTAN CILEXETIL:Ear infection:0.17), (CANDESARTAN CILEXETILHYDROCHLOROTHIAZIDE:Ear infection:0.03), (CHOLESTYRAMINE:Ear infection:0.67), (CORTISONEHYDROCORTISONE:Ear infection:0.49), (DASATINIB:Ear infection:0.5), (DOCUSATE:Ear infection:0.14), (ELETRIPTAN HYDROBROMIDE:Ear infection:0.32), (ENALAPRIL MALEATE:Ear infection:0.36), (ENOXAPARIN:Ear infection:0.83), (ESZOPICLONE:Ear infection:0.79), (EZETIMIBESIMVASTATIN:Ear infection:0.05), (FERROUS SULFATEFERROUS SULFATE, DRIED:Ear infection:1.28), (FINGOLIMOD HYDROCHLORIDE:Ear infection:0.68), (FLUTICASONE:Ear infection:0.11), (HYDROCHLOROTHIAZIDELISINOPRIL:Ear infection:0.65), (HYDROXYCHLOROQUINE DIPHOSPHATE:Ear infection:0.06), (IDELALISIB:Ear infection:0.04), (ILOPROST:Ear infection:0.08), (IMATINIB MESYLATE:Ear infection:0.12), (INDOMETHACIN:Ear infection:0.37), (ISOSORBIDE:Ear infection:0.07), (ISOSORBIDE MONONITRATE:Ear infection:0.35), (LABETALOLLABETALOL HYDROCHLORIDE:Ear infection:0.39), (MEROPENEM:Ear infection:0.07), (METAXALONE:Ear infection:0.27), (METHADONE HYDROCHLORIDE:Ear infection:0.07), (METHYLPREDNISOLONE ACETATE:Ear infection:0.42), (NEBIVOLOL:Ear infection:0.4), (NILOTINIB:Ear infection:0.55), (NORTRIPTYLINE:Ear infection:0.2), (PANTOPRAZOLE MAGNESIUM:Ear infection:0.32), (PAROXETINEPAROXETINE HYDROCHLORIDE:Ear infection:0.21), (PEGINTERFERON BETA-1A:Ear infection:0.05), (PRIMIDONE:Ear infection:0.59), (PYRAZINAMIDE:Ear infection:0.66), (RABEPRAZOLE:Ear infection:0.13), (RIZATRIPTAN BENZOATE:Ear infection:0.25), (ROFLUMILAST:Ear infection:0), (ROPINIROLE HYDROCHLORIDE:Ear infection:0.01), (SACUBITRILVALSARTAN:Ear infection:0.7), (SODIUM BICARBONATE:Ear infection:0.14), (SUMATRIPTANSUMATRIPTAN SUCCINATE:Ear infection:0.19), (TERBUTALINE SULFATE:Ear infection:0.17), (TETRACYCLINE:Ear infection:0.23), (TOFACITINIB CITRATE:Ear infection:0.37), (TORSEMIDE:Ear infection:0.21), (VERAPAMIL HYDROCHLORIDE:Ear infection:0.77), (ATENOLOL:Ear pain:0.52), (CLINDAMYCINCLINDAMYCIN PHOSPHATE:Ear pain:0.03), (COLCHICINE:Ear pain:0.03), (DIGOXIN:Ear pain:0.5), (DOCUSATE SODIUM:Ear pain:0.33), (EPINEPHRINE:Ear pain:0.02), (EVOLOCUMAB:Ear pain:0.24), (FERROUS SULFATE:Ear pain:0.18), (GLIMEPIRIDE:Ear pain:0), (IRON:Ear pain:0.38), (LANSOPRAZOLE:Ear pain:0.51), (TERIFLUNOMIDE:Ear pain:0.27), (TOFACITINIB CITRATE:Ear pain:0.11), (WARFARIN:Ear pain:0.84), (BUDESONIDE:Echocardiogram:0.45), (CLINDAMYCINCLINDAMYCIN PHOSPHATE:Echocardiogram:0.1), (DEXLANSOPRAZOLE:Echocardiogram:0.26), (ENALAP RIL:Echocardiogram:0.99), (ESCITALOPRAM OXALATE:Echocardiogram:0.4), (EVOLOCUMAB:Echocardiogram:0.08), (FERROUS SULFATE:Echocardiogram:0.3), (IBRUTINIB:Echocardiogram:0.34), (ILOPROST:Echocardiogram: 0.1), (PANTOPRAZOLE:Echocardiogram:0.21), (UMECLIDINIUM BROMIDEVILANTEROL TRIFENATATE:Echocardiogram:0.03), (UNSPECIFIED INGREDIENT:Echocardiogram:0.93), (VERAPAMIL HYDROCHLORIDE:Echocardiogram:0.44), (.ALPHA.1-PROTEINASE INHIBITOR HUMAN:Eczema:1.67), (ACENOCOUMAROL:Eczema:0.14), (ACETAMINOPHENOXYCOD ONE HYDROCHLORIDEOXYCODONE TEREPHTHALATE:Eczema:0.33), (ACETAMINOPHENOXYCODONEOXYCODONE HYDROCHLORIDE:Eczema:0.68), (ADENINE:Eczema:0.16), (ADENOSINE TRIPHOSPHATE:Eczema:0.3), (AGALSIDASE BETA:Eczema:0.15), (ALEMTUZUMAB:Eczema:0.49), (AMBROXOLCLENBUTEROL:Eczema: 0.41), (AMEZINIUM METILSULFATE:Eczema:0.91), (AMILORIDE HYDROCHLORIDEHYDROCHLOROTHIAZIDE:Eczema:0.01), (AMISULPRIDE:Eczema:0.11), (AMLODIPINE BESYLATEATORVASTATIN CALCIUM:Eczema:0.17), (AMLODIPINE BESYLATEHYDROCHLOROTHIAZIDEVALSARTAN:Eczema:0.05), (AMMONIUM LACTATE:Eczema:0.22), (BENZBROMARONE:Eczema:0.16), (BEPRIDIL:Eczema:0.46), (BE RAPROST:Eczema:0.01), (BETAMETHASONECALCIPOTRIENE:Eczema:0.71), (BIFIDOBA CTERIUM LONGUM SUBSP. INFANTIS:Eczema:0.29), (BUPRENORPHINE HYDROCHLORIDE:Eczema:0.66), (BUPRENORPHINE HYDROCHLORIDENALOXONE HYDROCHLORIDE:Eczema:0.66), (CALCITONIN SALMON:Eczema:0.42), (CALCIUM ACETATE:Eczema:0.36), (CALCIUM CARBONATEVITAMIN D:Eczema:0.17), (CALCIUM POLYCARBOPHIL:Eczema:0.16), (CALCIUMMAGNESIUM:Eczema:0.4), (CALCIUMMAG NESIUMZINC:Eczema:0.04), (CAMPHORMENTHOLMETHYL SALICYLATE:Eczema:0.59), (CANAGLIFLOZINMETFORMIN HYDROCHLORIDE:Eczema:0.32), (CAPSAICIN:Eczema:0.26), (CARVEDILOL PHOSPHATE:Eczema:1.29), (CEFDITOREN PIVOXIL:Eczema:1.54), (CEFOTAXIME SODIUM:Eczema:0.96), (CELLULASEDIASTASELIPASE:Eczema:0.36), (CEPHARANTHI NE:Eczema:0.54), (CETUXIMAB:Eczema:0.87), (CHLORDIAZEPOXIDE HYDROCHLORIDECLIDINIUM BROMIDE:Eczema:0.04), (CILASTATIN SODIUMIMIPENEM:Eczema:0.49), (COLESEVELAM HYDROCHLORIDE:Eczema:0.33), (COLISTIMETHATE SODIUM:Eczema:0.01), (CORTICOTROPIN:Eczema:0.45), (CRIZOTINIB:Eczema:0.44), (DAB RAFENIB:Eczema:0.15), (DALTEPARIN:Eczema:0.73), (DANAZOL:Eczema:0.09), (DAPAGLI FLOZIN PROPANEDIOL:Eczema:0.29), (DASABUVIROMBITASVIRPARITAPREVIRRITONAVIR:Eczema:0.06), (DEFLAZACORT:Eczema:1.27), (DEVICE:Eczema:0.12), (DIVALPROEX SODIUM:Eczema:1.22), (DORZOLAMIDE HYDROCHLORIDE:Eczema:0.08), (ECONAZOLE:Eczema:0.67), (EDOXABAN TOSYLATE:Eczema:1.27), (EFINACONAZOLE:Eczema:0.19), (EMPAGLIFLOZINLINAGLI PTIN:Eczema:0.17), (ERYTHROMYCIN LACTOBIONATE:Eczema:0.17), (ESOMEPRAZOLE MAGNESIUMNAPROXEN:Eczema:0.43), (ESTROGENS, CONJUGATEDMEDROXYPROGESTERONE ACETATE:Eczema:0.54), (ETHYL ICOSAPENTATEICOSAPENT:Eczema:0.36), (ETORICOXIB:Eczema:0.66), (ETRETINATE: Eczema:1.92), (EVOLOCUMAB:Eczema:1.4), (EZETIMIBESIMVASTATIN:Eczema:0.83), (F AROPENEM SODIUM:Eczema:0.59), (FENOTEROL HYDROBROMIDE:Eczema:0.14), (FERROUS FUMARATE:Eczema:0.22), (FESOTERODINE FUMARATE:Eczema:0.82), (FINGOLIMOD HYDROCHLORIDE:Eczema:0.37), (FISH OIL\OMEGA-3 FATTY ACIDS:Eczema:0.03), (FLAVIN ADENINE DINUCLEOTIDE:Eczema:1.46), (FLECAINIDE:Eczema:1.18), (FLUDROCORTISONE ACETATE:Eczema:0.26), (FLUNITRAZEPAM:Eczema:0.9), (FLUTICASONE PROPIONATEFORMOTEROL FUMARATE:Eczema:0.18), (FOSFOMYCIN CALCIUM:Eczema:0.61), (FULVESTRANT:Eczema:0.31), (FURSULTIAMINE:Eczema:0.12), (GIMERACILOTERACILTEGAFUR:Eczema:0.6), (GLYCOPYRROLATEINDACATEROL MALEATE:Eczema:0.07), (GLYCOPYRRONIUM:Eczema:0.69), (GLYCOPYRRONIUMIND ACATEROL:Eczema:0.93), (GUANABENZ ACETATE:Eczema:0.58), (GUANFACINE:Eczema:0.12), (HERBALSSAW PALMETTO:Eczema:0.04), (HYDROXYCHLOROQUINE DIPHOSPHATE:Eczema:0.74), (HYDROXYUREA:Eczema:0.05), (IFENPRODIL:Eczema:0.01), (ILOPROST:Eczema:0.6), (IMATINIB MESYLATE:Eczema:0.54), (IMIDAPRIL:Eczema:0.51), (IMMUNE GLOBULIN NOS:Eczema:0.24), (INDAPAMIDEPERINDOPRIL:Eczema:0.63), (INSULIN BEEF:Eczema:0.02), (INSULIN DEGLUDEC:Eczema:0.01), (INTERFERON GAMMA-1B:Eczema:0.09), (IODINE:Eczema:0.02), (IRINOTECAN:Eczema:0.34), (IRINOTECAN HYDROCHLORIDE:Eczema:0.67), (IXAZOMIB:Eczema:0.02), (LAPATINIB DITOSYLATE:Eczema:0.33), (LERCANIDIPINE:Eczema:0.94), (LETROZOLE:Eczema:0.68), (LEVALBUTEROL:Eczema:0.45), (LEVOCABASTINE HYDROCHLORIDE:Eczema:0.83), (LINEZOLID:Eczema:0.26), (LIOTHYRONINE:Eczema:0.17), (LIOTHYRONINE SODIUM:Eczema:0.32), (LOMITAPIDE MESYLATE:Eczema:0.25), (LUMACAFTOR:Eczema:0.22), (MEDROXYPROGESTERONE: Eczema:0.07), (MEPERIDINE HYDROCHLORIDE:Eczema:0.15), (MEPOLIZUMAB:Eczema:1.25), (METFORMIN HYDROCHLORIDEVILDAGLIPTIN:Eczema:0.13), (METHENAMINE HIPPURATE:Eczema:0.83), (METHENOLONE ENANTHATE:Eczema:0.13), (METHIMAZOLE:Eczema:0.81), (MEXILETINE HYDROCHLORIDE:Eczema:0.19), (MILK THISTLE:Eczema:0.06), (MOSAPRIDE CITRATE DIHYDRATE:Eczema:0.18), (MOXIFLOXACIN:Eczema:0.8), (NADOLOL:Eczema:0.5), (NAL OXONE HYDROCHLORIDEOXYCODONE HYDROCHLORIDE:Eczema:0.9), (NALOXONE HYDROCHLORIDETILIDINE HYDROCHLORIDE:Eczema:0.05), (NICARDIPINE:Eczema:0.14), (NICOTINE:Eczema:0.63), (NINTEDANIB:Eczema:0.91), (NITROFURANTOINNITROFURANTOIN MONOHYDRATE:Eczema:0.05), (OLMESARTAN:Eczema:0.11), (PAMIDRONATE DISODIUM:Eczema:0.14), (PAZOPANIB HYDROCHLORIDE:Eczema:1.32), (PEMETREXED DISODIUM:Eczema:0.86), (PENICILLIN:Eczema:0.93), (PENICILLIN G:Eczema:0.07), (PENTOXIFYLLINE:Eczema:0.64), (PHENOBARBITAL:Eczema:0.42), (PHE NYTOIN:Eczema:0.01), (PILSICAINIDE HYDROCHLORIDE:Eczema:0.76), (PIROXICAM:Eczema:0.46), (POSACONAZOLE:Eczema: 0.08), (POTASSIUM CITRATETRISODIUM CITRATE DIHYDRATE:Eczema:0.64), (POTASSIUM IODIDE:Eczema:0.02), (PROPAFENONE HYDROCHLORIDE:Eczema:0.05), (PYRIDOSTIGMINE BROMIDE:Eczema:0.41), (PYRIDOXINE:Eczema:0.4), (QUINACRINE:Eczema:0.26), (RIFAM PIN:Eczema:0.78), (RIFAXIMIN:Eczema:0.05), (RIVASTIGMINE:Eczema:0.16), (RIZATRIPT AN:Eczema:0.22), (SACUBITRILVALSARTAN:Eczema:0.34), (SARPOGRELATE:Eczema:1.58), (SARPOGRELATE HYDROCHLORIDE:Eczema:0.3), (SERRAPEPTASE:Eczema:0.08), (SEVELAMER HYDROCHLORIDE:Eczema:0.25), (SODIUM FLUORIDE:Eczema:0.47), (STREPTOMYCIN SULFATE:Eczema:0.57), (SULFAMETHOXAZOLE:Eczema:0.83), (SUMATRIPTANSUMAT RIPTAN SUCCINATE:Eczema:1.37), (SUPLATAST TOSILATE:Eczema:0.39), (TEMOCAPRIL HYDROCHLORIDE:Eczema:0.65), (THEOPHYLLINE:Eczema:0.07), (THYROID, PORCINE:Eczema:0.57), (TINZAPARIN SODIUM:Eczema:0.4), (TIPIRACIL HYDROCHLORIDETRIFLURIDINE:Eczema:0.23), (TIZANIDINE:Eczema:0.87), (TOCOPHE ROL:Eczema:0.37), (TOLTERODINE TARTRATE:Eczema:0.65), (TRAMETINIB:Eczema:0.23), (UMECLIDINIUM BROMIDE:Eczema:0.5), (UMECLIDINIUM BROMIDEVILANTEROL TRIFENATATE:Eczema:1.03), (VALGANCICLOVIR HYDROCHLORIDE:Eczema:0.95), (VINORELBINE TARTRATE:Eczema:0.05), (VONOPRAZAN:Eczema:0.03), (WARFARIN POTASSIUM:Eczema:0.7), (ZAFIRLUKAST:Eczema:0.21), (ZILEUTON:Eczema:0.6), (ZONIS AMIDE:Eczema:0.09), (ALBUTEROL SULFATEIPRATROPIUM BROMIDE:Electrolyte imbalance:0.31), (AMIODARONE:Electrolyte imbalance:0.71), (AMITRIPTYLINE:Electrolyte imbalance:0.26), (AMPHETAMINE ASPARTATEAMPHETAMINE SULFATEDEXTROAMPHETAMINE SACCHARATEDEXTROAMPHETAMINE SULFATE:Electrolyte imbalance:0.09), (ANASTROZOLE:Electrolyte imbalance:0.26), (BUPROPION HYDROCHLORIDE:Electrolyte imbalance:0.82), (CALCIUM CARBONATECHOLECALCIFEROL:Electrolyte imbalance:0.6), (CERTOLIZUMAB PEGOL:Electrolyte imbalance:0.6), (CHOLESTYRAMINE:Electrolyte imbalance:0.02), (CITALOPRAM HYDROBROMIDE:Electrolyte imbalance:0.69), (CLOTRIMAZOLE:Electrolyte imbalance:0), (CLOZAPINE:Electrolyte imbalance:0.46), (DULOXETINE:Electrolyte imbalance:0.05), (EVOLOCUMAB:Electrolyte imbalance:0.26), (FLECAINIDE:Electrolyte imbalance:0.02), (FLUTICASONE:Electrolyte imbalance:0.06), (FLUTICASONE FUROATEVILANTEROL TRIFENATATE:Electrolyte imbalance:0.03), (HYDROCHLOROTHIAZIDELISINOPRIL:Electrolyte imbalance:0.31), (HYDROXYZINE PAMOATE:Electrolyte imbalance:0.16), (IBRUTINIB:Electrolyte imbalance:1.24), (ILOPROST:Electrolyte imbalance:0.2), (IPRATROPIUM:Electrolyte imbalance:0.09), (IRBESARTAN:Electrolyte imbalance:0.08), (ITRACONAZOLE:Electrolyte imbalance:0.02), (LEDIPASVIRSOFOSBUVIR:Electrolyte imbalance:0.17), (LERCANIDIPINE:Electrolyte imbalance:0.13), (MELATONIN:Electrolyte imbalance:0.13), (NILOTINIB:Electrolyte imbalance:0.36), (POLYETHYLENE GLYCOLS:Electrolyte imbalance:0.61), (PONATINIB:Electrolyte imbalance:0.08), (PRAVASTATIN:Electrolyte imbalance:0.63), (PRIMIDONE:Electrolyte imbalance:0.3), (RANOLAZINE:Electrolyte imbalance:0.53), (SACUBITRILVALSARTAN:Electrolyte imbalance:0.25), (SOTALOL:Electrolyte imbalance:0.05), (TELMISARTAN:Electrolyte imbalance:0.16), (TOCILIZUMAB:Electrolyte imbalance:0.02), (TOPIRAMATE:Electrolyte imbalance:0.81), (VALGANCICLOVIR HYDROCHLORIDE:Electrolyte imbalance:0.64), (VENLAFAXINE HYDROCHLORIDE:Electrolyte imbalance:0.46), (.ALPHA.1-PROTEINASE INHIBITOR HUMAN:Electrolyte substitution therapy:0.49), (ACETAMINOPHENOXYCODONEOXYCODONE HYDROCHLORIDE:Electrolyte substitution therapy:0.66), (ADO-TRASTUZUMAB EMTANSINE:Electrolyte substitution therapy:0.27), (AGALSIDASE BETA:Electrolyte substitution therapy:0.03), (ALCOHOL:Electrolyte substitution therapy:0.33), (ALENDRONIC ACID:Electrolyte substitution therapy:0.42), (AMISULPRIDE:Electrolyte substitution therapy:0.47), (AMLODIPINEVALSARTAN:Electrolyte substitution therapy:0.02),(ARMODAFINIL:Electrolyte substitution therapy:1.02), (ATRACURIUM:Electrolyte substitution therapy:0.39), (ATRACURIUM BESYLATE:Electrolyte substitution therapy:0.14), (BACITRACIN:Electrolyte substitution therapy:0.11), (BLINATUMOMAB:Electrolyte substitution therapy:0.45), (BRINZOLAMIDE:Electrolyte substitution therapy:0.69), (CALCITONIN SALMON:Electrolyte substitution therapy:0.3), (CANAGLIFLOZINMETFORMIN HYDROCHLORIDE:Electrolyte substitution therapy:0.51), (CANDESARTAN:Electrolyte substitution therapy:1.28), (CANDESARTAN CILEXETILHYDROCHLOROTHIAZIDE:Electrolyte substitution therapy:0.92), (CARISOPRODOL:Electrolyte substitution therapy:0.37), (CHLORAMBUCIL:Electrolyte substitution therapy:0.01), (CHLOROTHIAZIDE:Electrolyte substitution therapy:0), (CHLORPHENIRAMINEHYDROCODONE:Electrolyte substitution therapy:0.1), (CINACALCET HYDROCHLORIDE:Electrolyte substitution therapy:0.53), (CLINDAMYCIN:Electrolyte substitution therapy:0.83), (CLOMIPRAMINE HYDROCHLORIDE:Electrolyte substitution therapy:0.18), (CLOZAPINE:Electrolyte substitution therapy:1.16), (COLISTIMETHATE SODIUM:Electrolyte substitution therapy:0.38), (COLISTIN:Electrolyte substitution therapy:0.16), (CRIZOTINIB:Electrolyte substitution therapy:0.19), (DACLIZUMAB:Electrolyte substitution therapy:0.33), (DALTEPARIN:Electrolyte substitution therapy:0.66), (DANAZOL:Electrolyte substitution therapy:0.14), (DESLORATADINE:Electrolyte substitution therapy:0.59), (DESONIDE:Electrolyte substitution therapy:0.14), (DIGITOXIN:Electrolyte substitution therapy:0.02), (DIPYRIDAMOLE:Electrolyte substitution therapy:0.05), (DIPYRONE:Electrolyte substitution therapy:0.2), (DONEPEZIL:Electrolyte substitution therapy:0.01), (DULAGLUTIDE:Electrolyte substitution therapy:0.02), (ELETRIPTAN HYDROBROMIDE:Electrolyte substitution therapy:0.86), (EMPAGLIFLOZINLINAGLIPTIN:Electrolyte substitution therapy:0.31), (ESMOLOL HYDROCHLORIDE:Electrolyte substitution therapy:0.7), (ESOMEPRAZOLE MAGNESIUMNAPROXEN:Electrolyte substitution therapy:0.19), (EVOLOCUMAB:Electrolyte substitution therapy:1.02), (FENOTEROL HYDROBROMIDEIPRATROPIUM BROMIDE:Electrolyte substitution therapy:0.07), (FESOTERODINE FUMARATE:Electrolyte substitution therapy:0.85), (FLUDROCORTISONE ACETATE:Electrolyte substitution therapy:0.09), (FORMOTEROL:Electrolyte substitution therapy:0.39), (FORMOTEROL FUMARATEMOMETASONE FUROATE:Electrolyte substitution therapy:0.48), (FUSIDIC ACID:Electrolyte substitution therapy:0.35), (GIMERACILOTERACILTEGAFUR:Electrolyte substitution therapy:0.08), (GLYCOPYRRONIUM:Electrolyte substitution therapy:0.34), (HERBALSTURMERIC:Electrolyte substitution therapy:0.27), (HYDROCHLOROTHIAZIDEIRBESARTAN:Electrolyte substitution therapy:0.71), (HYDROCHLOROTHIAZIDETELMISARTAN:Electrolyte substitution therapy:0.11), (HYOSCYAMINE:Electrolyte substitution therapy:0.51), (INDACATEROL:Electrolyte substitution therapy:0.21), (INDAPAMIDEPERINDOPRIL:Electrolyte substitution therapy:0.56), (INSULIN DEGLUDEC:Electrolyte substitution therapy:0.08), (INTERFERON GAMMA-1B:Electrolyte substitution therapy:0.06), (ISOFLURANE:Electrolyte substitution therapy:0.82), (KETOROLACKETOROLAC TROMETHAMINE:Electrolyte substitution therapy:0.33), (LABETALOLLABETALOL HYDROCHLORIDE:Electrolyte substitution therapy:1.38), (LERCANIDIPINE:Electrolyte substitution therapy:1.1), (LERCANIDIPINE HYDROCHLORIDE:Electrolyte substitution therapy:0.34), (LETROZOLE:Electrolyte substitution therapy:0.11), (LISDEXAMFETAMINE DIMESYLATE:Electrolyte substitution therapy:0.86), (MEPOLIZUMAB:Electrolyte substitution therapy:0.19), (METAMIZOLE:Electrolyte substitution therapy:0.05), (METHYLPREDNISOLONE ACETATE:Electrolyte substitution therapy:0.45), (MIDODRINE:Electrolyte substitution therapy:0.6), (MIRABEGRON:Electrolyte substitution therapy:0.22), (MUPIROCINMUPIROCIN CALCIUM:Electrolyte substitution therapy:0.24), (NABUMETONE:Electrolyte substitution therapy:0.3), (NEBIVOLOL:Electrolyte substitution therapy:0.8), (NILOTINIB:Electrolyte substitution therapy:1.5), (NIRAPARIB:Electrolyte substitution therapy:0.05), (OLODATEROL HYDROCHLORIDETIOTROPIUM BROMIDE MONOHYDRATE:Electrolyte substitution therapy:0.74), (OLOPATADINE:Electrolyte substitution therapy:0.37), (OMEPRAZOLE SODIUM:Electrolyte substitution therapy:0.16), (OSELTAMIVIR:Electrolyte substitution therapy:0.37), (PANTOPRAZOLE MAGNESIUM:Electrolyte substitution therapy:1.58), (PATIROMER:Electrolyte substitution therapy:0.09), (PEGINTERFERON BETA-1A:Electrolyte substitution therapy:0.23), (PEMETREXED DISODIUM:Electrolyte substitution therapy:0.21), (PITAVASTATIN CALCIUM:Electrolyte substitution therapy:0.38), (PREDNISOLONEPREDNISOLONE ACETATEPREDNISOLONE SODIUM SUCCINATE:Electrolyte substitution therapy:0.65), (PROGESTERONE:Electrolyte substitution therapy:0.32), (PROPRANOLOLPROPRANOLOL HYDROCHLORIDE:Electrolyte substitution therapy:0.18), (QUETIAPINE:Electrolyte substitution therapy:1.14), (QUINAPRIL:Electrolyte substitution therapy:0.06), (REMIFENTANIL:Electrolyte substitution therapy:0.56), (RIZATRIPTAN BENZOATE:Electrolyte substitution therapy:0.6), (ROFLUMILAST:Electrolyte substitution therapy:0.86), (SALMETEROL XINAFOATE:Electrolyte substitution therapy:0.11), (SECUKINUMAB:Electrolyte substitution therapy:0.44), (SUFENTANIL:Electrolyte substitution therapy:0.58), (SUFENTANIL CITRATE:Electrolyte substitution therapy:0.13), (SUMATRIPTANSUMATRIPTAN SUCCINATE:Electrolyte substitution therapy:0.99), (SUNITINIB MALATE:Electrolyte substitution therapy:0.3), (TERBUTALINE:Electrolyte substitution therapy:0.42), (TERIFLUNOMIDE:Electrolyte substitution therapy:0.22), (TETRACYCLINE:Electrolyte substitution therapy:0.59), (THALIDOMIDE:Electrolyte substitution therapy:0.98), (THIOPENTAL:Electrolyte substitution therapy:0.23), (TOCILIZUMAB:Electrolyte substitution therapy:0.34), (TOCOPHEROL:Electrolyte substitution therapy:0.22), (UMECLIDINIUM BROMIDE:Electrolyte substitution therapy:0.01), (VALGANCICLOVIR HYDROCHLORIDE:Electrolyte substitution therapy:1.11), (VITAMIN A:Electrolyte substitution therapy:0.41), (ZINC:Electrolyte substitution therapy:0.37), (ACARBOSE:Embolism:0.29), (ALCOHOL:Embolism:0.18), (ALFACALCIDOL: Embolism:0.51), (AMANTADINE:Embolism:0.09), (AMISULPRIDE:Embolism:0.06), (AMITRI PTYLINE HYDROCHLORIDE:Embolism:0.18),(ARMODAFINIL:Embolism:0.3), (AZITHROMYCIN DIHYDRATE:Embolism:0.06), (BECLOMETHASONE DIPROPIONATE:Embolism:0.52), (BUDESONIDEFORMOTEROL FUMARATE:Embolism:0.27), (CALCITONIN:Embolism:0.02), (CALCIUM ACETATE:Embolism:0.78), (CAPTOPRIL:Embolism:0.09), (CARVEDILOL PHOSPHATE:Embolism:0.52), (CERTOLIZUMAB PEGOL:Embolism:0.78), (CHOLESTYRAMINE:Embolism:0.7), (CLINDAMYCIN:Embolism:00.11), (CLOTRIMAZOLE:Embolism:0.72), (CLOZAPINE:Embolism:1.14), (CORTISONEHYDR OCORTISONE:Embolism:0.52), (CRIZOTINIB:Embolism:0.38), (DABIGATRAN:Embolism:0.6), (DACLIZUMAB:Embolism:0.16), (DEVICE:Embolism:0.44), (DEXLANSOPRAZOLE:Embolism: 1.19), (DIGITOXIN:Embolism:0.47), (DOFETILIDE:Embolism:0.34), (DRONABINOL:Embolism: 0.27), (ELETRIPTAN HYDROBROMIDE:Embolism:0.17), (EVOLOCUMAB:Embolism:0.63), (FLUOXETINEFLU OXETINE HYDROCHLORIDE:Embolism:0.3), (FLUTICASONE:Embolism:0.52), (FLUTICASONE FUROATE:Embolism:0.16), (FLUTICASONE FUROATESALMETEROL XINAFOATE:Embolism:0.5), (FORMOTEROL FUMARATE DIHYDRATEMOMETASONE FUROATE:Embolism:0.28), (GLYCOPYRRONIUMINDACATEROL:Embolism:0.5), (HYDR OCHLOROTHIAZIDEIRBESARTAN:Embolism:0.64), (IBRUTINIB:Embolism:1.59), (IMIPR AMINE HYDROCHLORIDE:Embolism:0.05), (INDAPAMIDEPERINDOPRIL:Embolism:0.88), (INSU LIN GLULISINE:Embolism:0.02), (IRINOTECAN HYDROCHLORIDE:Embolism:0.3), (LEDIPASVIRSOFOSBUVIR:Embolism:0.67), (LERCA NIDIPINE HYDROCHLORIDE:Embolism:0.92), (LIDOCAINE HYDROCHLORIDE:Embolism:0.21), (LIDOCAINEPRILOCAINE:Embolism:0.53), (LISDEX AMFETAMINE DIMESYLATE:Embolism:0.34), (LITHIUM CARBONATE:Embolism:1.02), (MELATONIN:Embolism:0.7), (MESNA:Embolism:0.14), (ME TAXALONE:Embolism:0.21), (METHENAMINE HIPPURATE:Embolism:0.28), (METHIMAZOLE:Embolism:0.14), (MIDODRINE:Embolism:0.31), (MOXIFLOXACIN:Embolism:0.07), (NICORANDIL:Embolism:0.04), (NINTEDANIB:Embolism: 0.34), (OLODATEROL HYDROCHLORIDETIOTROPIUM BROMIDE MONOHYDRATE:Embolism:0.14), (OMEPRAZOLE SODIUM:Embolism:0), (OXCARBAZEPINE:Embolism:1.06), (PANTOPRAZOLE MAGNESIUM:Embolism:0.77), (PEGINTERFERON BETA-1A:Embolism:0.21), (PEMETREXED DISODIUM:Embolism:0.36), (PENICILLIN:Embolism:0.25), (PERTUZUMAB:Embolism:0.09), (PHENOBARBITAL:Embolism:0.53), (POSACONAZOLE:Embolism:0), (REGORAFENIB: Embolism:0.22), (RISEDRONATE SODIUM:Embolism:0.09), (SCOPOLAMINE:Embolism:0.57), (SECUKINUMAB:Embolism:0.15), (SELEXIPAG:Embolism:0.42), (TERBUTALINE SULFATE:Embolism:0.2), (TERIFLUNOMIDE:Embolism:0.31), (TOCILIZUMAB:Embolism:0.06), (TRAMETINIB:Embolism:0), (VALGANCICLOVIR HYDROCHLORIDE:Embolism:0.69), (VANCOMYCIN HYDROCHLORIDE:Embolism:0.25), (.ALPHA.-TOCOPHEROL:Embolism venous:0.54), (ACARBOSE:Embolism venous:0.56), (ACETAMINOPHENOXYCODONEOXYCODONE HYDROCHLORIDE:Embolism venous:0.3), (ACETAZOLAMIDE:Embolism venous:0.88), (ADO-TRASTUZUMAB EMTANSINE:Embolism venous:0.39), (AGALSIDASE BETA:Embolism venous:0.51), (AMISULPRIDE:Embolism venous:0.44),(ARMODAFINIL:Embolism venous:0.41), (ATROPINE SULFATEDIPHENOXYLATE HYDROCHLORIDE:Embolism venous:0.11), (AXITINIB:Embolism venous:0.06), (BENAZEPRILBENAZEPRIL HYDROCHLORIDE:Embolism venous:0.38), (BUDESONIDEFORMOTEROL FUMARATE:Embolism venous:0.86), (BUPRENORPHINE HYDROCHLORIDE:Embolism venous:0.99), (CALCITONIN:Embolism venous:0.25), (CALCITONIN SALMON:Embolism venous:0.39), (CANAGLIFLOZINMETFORMIN HYDROCHLORIDE:Embolism venous:0.02), (CARBIMAZOLE:Embolism venous:0.1), (CARFILZOMIB:Embolism venous:0.82), (CARVEDILOL PHOSPHATE:Embolism venous:1.16), (CEFAZOLIN SODIUM:Embolism venous:0.07), (CEFOTAXIME SODIUM:Embolism venous:0.18), (CETUXIMAB:Embolism venous:0.7), (CHLORPROMAZINE:Embolism venous:0.12), (CICLESONIDE:Embolism venous:0.33), (CILASTATIN SODIUMIMIPENEM:Embolism venous:0.61), (CLINDAMYCIN:Embolism venous:0.57), (CYAMEMAZINE:Embolism venous:0.12), (DANAZOL:Embolism venous:0.79), (DAPAGLIFLOZIN PROPANEDIOL:Embolism venous:0.05), (DEFLAZACORT:Embolism venous:0.64), (DEVICE:Embolism venous:0.26), (DEXAMETHASONE ACETATE:Embolism venous:0.07), (DIMENHYDRINATE:Embolism venous:0.69), (DOFETILIDE:Embolism venous:0.58), (DRONABINOL:Embolism venous:0.27), (DUTASTERIDETAMSULOSIN HYDROCHLORIDE:Embolism venous:0.35), (ELETRIPTAN HYDROBROMIDE:Embolism venous:0.75), (ESTROGENS, CONJUGATEDMEDROXYPROGESTERONE ACETATE:Embolism venous:0.08), (EVOLOCUMAB:Embolism venous:0.32), (FESOTERODINE FUMARATE:Embolism venous:1.1), (FLUTICASONE:Embolism venous:0.68), (FLUTICASONE FUROATEVILANTEROL TRIFENATATE:Embolism venous:1.2), (FORMOTEROL FUMARATEMOMETASONE FUROATE:Embolism venous:0.06), (FULVESTRANT:Embolism venous:0.12), (GLYCOPYRRONIUM:Embolism venous:0.08), (HYOSCYAMINE:Embolism venous:0.01), (IDARUCIZUMAB:Embolism venous:0), (IMIPRAMINE HYDROCHLORIDE:Embolism venous:0.44), (INSULIN BEEF:Embolism venous:0.1), (INTERFERON ALFA-2A:Embolism venous:0.06), (LABETALOL HYDROCHLORIDE:Embolism venous:0.64), (LEDIPASVIRSOFOSBUVIR:Embolism venous:1.08), (LERCANIDIPINE HYDROCHLORIDE:Embolism venous:1.64), (LEVOLEUCOVORIN CALCIUM:Embolism venous:0.5), (LEVOTHYROXINE SODIUMPOTASSIUM IODIDE:Embolism venous:0.05), (LISDEXAMFETAMINE DIMESYLATE:Embolism venous:0.2), (MELPERONE:Embolism venous:0.1), (MEPOLIZUMAB:Embolism venous:0.03), (METAXALONE:Embolism venous:0.25), (METHYLPREDNISOLONE ACETATE:Embolism venous:0.69), (MINOCYCLINE HYDROCHLORIDE:Embolism venous:0.04), (MINOCYCLINEMINOCYCLINE HYDROCHLORIDE:Embolism venous:0.29), (MODAFINIL:Embolism venous:0.33), (NADOLOL:Embolism venous:0.32), (NATEGLINIDE:Embolism venous:0.17), (NICARDIPINE:Embolism venous:0.36), (NILOTINIB:Embolism venous:2.43), (NINTEDANIB:Embolism venous:0.54), (NITROFURANTOINNITROFURANTOIN MONOHYDRATE:Embolism venous:0.23), (OLODATEROL HYDROCHLORIDETIOTROPIUM BROMIDE MONOHYDRATE:Embolism venous:0.86), (PANITUMUMAB:Embolism venous:0.29), (PANTOPRAZOLE MAGNESIUM:Embolism venous:1.31), (PAZOPANIB HYDROCHLORIDE:Embolism venous:0.55), (PEMETREXED:Embolism venous:0.23), (PROGESTERONE:Embolism venous:0.03), (PROPAFENONE HYDROCHLORIDE:Embolism venous:0.58), (PYRIDOXINE:Embolism venous:0.23), (RIVASTIGMINE:Embolism venous:0.51), (SACUBITRILVALSARTAN:Embolism venous:1.18), (SECUKINUMAB:Embolism venous:0.2), (SEVELAMER CARBONATE:Embolism venous:0.07), (SODIUM OXYBATE:Embolism venous:0.41), (SUNITINIB MALATE:Embolism venous:0.24), (TERBINAFINE HYDROCHLORIDE:Embolism venous:0.42), (TICLOPIDINE:Embolism venous:0.06), (TOCILIZUMAB:Embolism venous:0.29), (TOFACITINIB CITRATE:Embolism venous:0.2), (UMECLIDINIUM BROMIDE:Embolism venous:0.02), (UMECLIDINIUM BROMIDEVILANTEROL TRIFENATATE:Embolism venous:0.32), (VALGANCICLOVIR HYDROCHLORIDE:Embolism venous:0.37), (VARENICLINE TARTRATE:Embolism venous:0.83), (VITAMIN A:Embolism venous:0.21), (VITAMIN B:Embolism venous:0.9), (ZINC:Embolism venous:0.02), (CERTOLIZUMAB PEGOL:Emotional disorder:0.12), (HYDROXYCHLOROQUINE SULFATE:Emotional disorder:0.14), (MAGNESIUM:Emotional disorder:0.09), (TERIFLUNOMIDE:Emotional disorder:0.07), (ZONISAMIDE:Emotional disorder:0.01), (ACARBOSE:Emphysema:0.55), (ADO-TRASTUZUMAB EMTANSINE:Emphysema:0.25), (AGALSIDASE BETA:Emphysema:0.42), (ALBIGLUTIDE:Emphysema:1.01), (AMANTADINE HYDROCHLORIDE:Emphysema:0.1), (AMLODIPINEVALSARTAN:Emphysema:0.38), (BET AMETHASONE DIPROPIONATECALCIPOTRIENECALCIPOTRIENE HYDRATE:Emphysema:0.06), (BUPRENORPHINE HYDROCHLORIDE:Emphysema:0.79), (CALCIUMMAGNESIUM:Emphysema:0.13), (CALC IUMMAGNESIUMZINC:Emphysema:0.08), (CANAGLIFLOZINMETFORMIN HYDROCHLORIDE:Emphysema:0.22), (CANDESARTAN CILEXETILHYDROCHLOROTHIAZIDE:Emphysema:1.34), (CARFILZOMIB:Emphysema:0.97), (CINACALCET HYDROCHLORIDE:Emphysema:0.93), (CLINDAMYCIN:Emphysema:0.65), (COD LIVER OIL:Emphysema:0.4), (CODEINE PHOSPHATEGUAIFENESIN:Emphysema:0.15), (CORTICOTROPIN:Emphysema:0.53), (DA LFAMPRIDINE:Emphysema:0.56), (DASATINIB:Emphysema:2.14), (DESONIDE:Emphysema: 0.14), (DIGITOXIN:Emphysema:0.24), (DIMENHYDRINATE:Emphysema:0.44), (DIPYRIDA MOLE:Emphysema:0.19), (DUTASTERIDETAMSULOSIN HYDROCHLORIDE:Emphysema:0.36), (EMPAGLIFLOZINLINAGLIPTIN:Emphysema:0.35), (ESTROGENS, CONJUGATEDMEDROXYPROGESTERONE ACETATE:Emphysema:0.48), (ETORICOXIB:Emphysema:0.78), (EVOLOCUMAB:Emphysema: 2.04), (FEXOFENADINEPSEUDOEPHEDRINE:Emphysema:0.44), (FLUOCINONIDE: Emphysema:0.35), (FORMOTEROL FUMARATEMOMETASONE FUROATE:Emphysema:1.29), (FULVESTRANT:Emphysema:0.61), (FUSIDIC ACID:Emphysema:0.06), (HERBALSTURMERIC:Emphysema:0.5), (ILOPROST:Emphysema: 1.41), (LACTASE:Emphysema:0.02), (LAPATINIB DITOSYLATE:Emphysema:0.05), (LERCANIDIPINE:Emphysema:0.86), (LERCANIDIPINE HYDROCHLORIDE:Emphysema:0.43), (LIOTHYRONINE SODIUM:Emphysema:0.07), (LISDEXAMFETAMINE DIMESYLATE:Emphysema:1.07), (LOMITAPIDE MESYLATE:Emphysema:0.43), (METHENAMINE HIPPURATE:Emphysema:0.87), (METHYLPREDNISOLONE ACETATE:Emphysema:0.78), (MIDODRINE:Emphysema:0.61), (MIDODRINE HYDROCHLORIDE:Emphysema:0.06), (MINOCYCLINEMINOCYCLINE HYDROCHLORIDE:Emphysema:0.42), (NALOXONE HYDROCHLORIDEOXYCODONE HYDROCHLORIDE:Emphysema:0.81), (NALTREXONE:Emphysema:0.23), (NATEGLINIDE: Emphysema:0.44), (OMEPRAZOLE SODIUM:Emphysema:0.11), (OXYBUTYNINOXYBUTYNIN CHLORIDE:Emphysema:0.02), (PATIROMER:Emphysema:0.34), (PAZOPANIB HYDROCHLORIDE:Emphysema:1), (PEGINTERFERON BETA-1A:Emphysema:0.6), (PENICILLIN:Emphysema:0.73), (PIMECROLIMUS:Emphysema:0.02), (P IROXICAM:Emphysema:0.49), (PONATINIB:Emphysema:0.59), (PREDNISOLONE ACETATE:Emphysema:0.58), (PROPAFENONE:Emphysema:0.21), (PYRIDOXINE:Emphysema: 0.31), (RIZATRIPTAN BENZOATE:Emphysema:0.75), (SACCHAROMYCES CEREVISIAE:Emphysema:0.04), (SCOPOLAMINE:Emphysema:0.82), (SELEXIPAG:Emphysema: 0.44), (SODIUM FLUORIDE:Emphysema:0.16), (TETRACYCLINE:Emphysema:0.41), (TIMOLOL MALEATE:Emphysema:0.35), (ZILEUTON:Emphysema:0.7), (ZONISAMIDE:Emphysema:0.18), (ETHAMBUTOL HYDROCHLORIDE:Encephalitis brain stem:0.71), (ZONISAMIDE:Encephalitis viral:0.01), (BUPROPION HYDROCHLORIDE:Encephalopathy:0.18), (DEXLANSOPRAZOLE:Encephalopathy:0.12), (DI GOXIN:Encephalopathy:0.36), (DILTIAZEM:Encephalopathy:0.07), (DILTIAZEM HYDROCHLORIDE:Encephalopathy:0.03), (FISH OIL:Encephalopathy:0.45), (IBRUTINIB:Encephalopathy:0.47), (LOVASTATIN:Encephalopathy: 0.06), (MELOXICAM:Encephalopathy:0.08), (NIFEDIPINE:Encephalopathy:0.07), (TIOTROP IUM BROMIDE MONOHYDRATE:Encephalopathy:0.15), (VALSARTAN:Encephalopathy:0.33), (AMPHETAM INE ASPARTATEAMPHETAMINE SULFATEDEXTROAMPHETAMINE SACCHARATEDEXTROAMPHETAMINE SULFATE:End stage renal disease:0.03), (BUSPIRONE HYDROCHLORIDE:End stage renal disease:0.05), (CALCIUM CARBONATECHOLECALCIFEROL:End stage renal disease:0.24), (CALCIUM CHLORIDEDEXTROSE MONOHYDRATEMAGNESIUM CHLORIDESODIUM CHLORIDESODIUM LACTATE:End stage renal disease:1.09), (CANDESARTAN CILEXETIL:End stage renal disease:0.48), (CARVEDILOL PHOSPHATE:End stage renal disease:0.13), (CERTOLIZUMAB PEGOL:End stage renal disease:0.73), (CLOTRIMAZOLE:End stage renal disease:0.39), (DASATINIB:End stage renal disease:0.26), (DULAGLUTIDE:End stage renal disease:0.26), (DULOXETINE:End stage renal disease:0.29), (DUTASTERIDE:End stage renal disease:0.06), (ENOXAPARIN:End stage renal disease:1.25), (EVOLOCUMAB:End stage renal disease:0.43), (FERROUS SULFATEFERROUS SULFATE, DRIED:End stage renal disease:1.1), (FLECAINIDE:End stage renal disease:0.02), (FLUTICASONE FUROATEVILANTEROL TRIFENATATE:End stage renal disease:0.21), (HYDROCHLOROTHIAZIDELISINOPRIL:End stage renal disease:0.28), (HYDROCHLOROTHIAZIDEVALSARTAN:End stage renal disease:0.07), (HYDROXYZINE PAMOATE:End stage renal disease:0.18), (HYOSCYAMINE:End stage renal disease:0.01), (ILOPROST:End stage renal disease:0.25), (LEVALBUTEROL HYDROCHLORIDE:End stage renal disease:0.51), (LISDEXAMFETAMINE DIMESYLATE:End stage renal disease:0.06), (MELOXICAM:End stage renal disease:0.79), (METFORMIN HYDROCHLORIDE:End stage renal disease:0.78), (NAPROXEN:End stage renal disease:0.76), (NAPROXEN SODIUM:End stage renal disease:0.15), (NITROFURANTOIN:End stage renal disease:0.12), (PANTOPRAZOLE MAGNESIUM:End stage renal disease:0.07), (PHENOBARBITAL:End stage renal disease:0.48), (PRAMIPEXOLE DIHYDROCHLORIDE:End stage renal disease:0.16), (PRIMIDONE:End stage renal disease:0.38), (SOTALOL:End stage renal disease:0.38), (SULFAMETHOXAZOLE:End stage renal disease:0.12), (TERIFLUNOMIDE:End stage renal disease:0.02), (THALIDOMIDE:End stage renal disease:0.09), (TIMOLOL:End stage renal disease:0.37), (TIZANIDINE:End stage renal disease:0.06), (TIZANIDINE HYDROCHLORIDE:End stage renal disease:0.13), (UMECLIDINIUM BROMIDEVILANTEROL TRIFENATATE:End stage renal disease:0.14), (VITAMIN B:End stage renal disease:0.28), (ZONISAMIDE:End stage renal disease:0.17), (AZITHROMYCIN ANHYDROUS:Endocarditis:0.24), (CASPOFUNGIN:Endocarditis:0.21), (CEPHALEXIN:Endocarditis: 0.19), (FLUTICASONE PROPIONATE:Endocarditis:0.02), (IBRUTINIB:Endocarditis:0.69), (ILOPROST:Endocarditis:0.18), (LEVOTHYROXINE SODIUM:Endocarditis:0.24), (NAPROXEN:Endocarditis:0.14), (OSELTAMIVIR:Endocarditis:00.25), (ACETAMINOPHENPROPOXYPHENE HYDROCHLORIDE:Endometriosis:0), (AMIODARONE:Endometriosis:0.31), (AZELASTINE: Endometriosis:0.22), (BETAMETHASONE DIPROPIONATECALCIPOTRIENECALCIPOTRIENE HYDRATE:Endometriosis:0.01), (BICALUTAMIDE:Endometriosis:0.3), (BISOPROLOL FUMARATE:Endometriosis:0.06), (BUDESONIDEFORMOTEROL FUMARATE:Endometriosis:0.09), (CANDESARTAN CILEXETIL:Endometriosis:0.52), (CANDESARTAN CILEXETILHYDROCHLOROTHIAZIDE:Endometriosis:0.15), (CARVEDILOL PHOSPHATE:Endometriosis:0.5), (CIMETIDINE:Endometriosis:0.31), (CLOZAPINE:Endometriosis: 0.06), (CORTICOTROPIN:Endometriosis:0.19), (DACLIZUMAB:Endometriosis:0.77), (DA SATINIB:Endometriosis:0.34), (DEFLAZACORT:Endometriosis:0.01), (DEXTROAMPHETAM INE SULFATE:Endometriosis:0.07), (DILTIAZEM:Endometriosis:0.53), (DIPHENHYDRAMINE: Endometriosis:1.11), (DOXAZOSIN MESYLATE:Endometriosis:0.01), (DUTASTERIDE:Endometriosis:0.09), (ENALAPRIL MALEATE:Endometriosis:0.59), (ENOXAPARIN:Endometriosis:0.52), (ESOMEPRAZOLE MAGNESIUMNAPROXEN:Endometriosis:0.48), (ESTRADIOL HEMIHYDRATE:Endometriosis:0.04), (EVOLOCUMAB:Endometriosis:1.02), (FERROUS SULFATE:Endometriosis:0.83), (FLECAINIDE:Endometriosis:0.03), (FLUTICASONE FUROATEVILANTEROL TRIFENATATE:Endometriosis:0.17), (HALOPERIDOL:Endometriosis:0.12), (HUMAN PAPILLOMAVIRUS QUADRIVALENT (TYPES 6, 11, 16, AND 18) VACCINE, RECOMBINANT:Endometriosis:0.06), (HYDROCHLOROTHIAZIDEIRBESARTAN:Endometriosis: 0.2), (HYDROCHLOROTHIAZIDELOSARTAN POTASSIUM:Endometriosis:0.39), (HYDROCORTISONE ACETATE:Endometriosis:0.21), (HYDROXYCHLOROQUINE DIPHOSPHATE:Endometriosis:0.6), (IBRUTINIB:Endometriosis:0.66), (INDAPAMIDE:Endometriosis: 0.02), (ISOSORBIDE MONONITRATE:Endometriosis:0.75), (LEVOMEFOLATE CALCIUMMETHYLCOBALAMINPYRIDOXAL PHOSPHATE ANHYDROUS:Endometriosis:0.04), (LIDOCAINE HYDROCHLORIDE:Endometriosis:0.07), (LOMITAPIDE MESYLATE:Endometriosis:0.05), (MENOTROPINS:Endometriosis:0.05), (METHENAMINE HIPPURATE:Endometriosis:0.26), (METHYLPREDNISOLONE ACETATE:Endometriosis:1.24), (MUPIROCIN:Endometriosis:0), (NILOTINIB:Endometriosis:00.05), (OXYGEN:Endometriosis:0.72), (PANTOPRAZOLE MAGNESIUM:Endometriosis:0.86), (PEGFILGRASTIM:Endometriosis:0.02), (PEGINTERFER ON BETA-1A:Endometriosis:0.45), (PHENOBARBITAL:Endometriosis:0.46), (PIROXICAM:Endometriosis: 0.08), (PRAVASTATIN:Endometriosis:1.25), (PROTRIPTYLINE HYDROCHLORIDE:Endometriosis:0.09), (PYRIDOXINE:Endometriosis:0.38), (ROFLUMILA ST:Endometriosis:0.27), (SELEXIPAG:Endometriosis:0.14), (SENNOSIDES:Endometriosis:0.02), (SULINDAC:Endometriosis:0.04), (TAMSULOSIN:Endometriosis:1.02), (TELMISARTAN: Endometriosis:0.48), (TETRACYCLINE:Endometriosis:0.52), (TIMOLOL:Endometriosis:0.13), (T OCOPHEROL:Endometriosis:0.06), (UMECLIDINIUM BROMIDEVILANTEROL TRIFENATATE:Endometriosis:0.4), (ZINC:Endometriosis:0.52), (AMPICILLIN:Endotracheal intubation:0.02), (BUPIVACAINE HYDROCHLORIDE:Endotracheal intubation:0.09), (CLINDAMYCINCLINDAMYCIN PHOSPHATE:Endotracheal intubation:0.21), (DILTIAZEM:Endotracheal intubation:0.03), (HYDROMORPHONE HYDROCHLORIDE:Endotracheal intubation:0.08), (METARAMINOL:Endotracheal intubation:0.05), (MOXIFLOXACIN HYDROCHLORIDE:Endotracheal intubation:0.02), (OXCARBAZEPINE:Endotracheal intubation:0.35), (PENTOBARBITAL:Endotracheal intubation:0.26), (ROPIVACAINE HYDROCHLORIDE:Endotracheal intubation:1.16), (TOPIRAMATE:Endotracheal intubation:0.15), (VANCOMYCIN HYDROCHLORIDE:Endotracheal intubation:0.17), (IBUPROFEN:Energy increased:0.02), (OSELTAMIVIR:Enterococcal infection:0.19), (RIFAMPIN:Enterococcus test positive:0.62), (EVEROLIMUS:Enterocolitis:0.3), (NILOTINIB:Enterocolitis:0.03), (RAMIPRIL: Enterocolitis:0.25), (TOPIRAMATE:Enterocolitis:0.13), (ATRACURIUM:Epidural anaesthesia:0.48), (ISOFLURANE:Epidural anaesthesia:0.53), (SUCCINYLCHOLINE CHLORIDE:Epidural anaesthesia:0.02), (VECURONIUM BROMIDE:Epidural anaesthesia:0.67), (ACETAMINOPHENCODEINE PHOSPHATE:Epigastric discomfort:0.2), (AMIODARONE:Epigastric discomfort:0.2), (BISOPROLOL FUMARATE:Epigastric discomfort:0.2), (BUPROPION HYDROCHLORIDE:Epigastric discomfort:0.51), (CERTOLIZUMAB PEGOL:Epigastric discomfort:0.28), (DILTIAZEM HYDROCHLORIDE:Epigastric discomfort:0.72), (ESCITALOPRAM OXALATE:Epigastric discomfort:0.54), (EVEROLIMUS:Epigastric discomfort:0.16), (EVOLOCUMAB:Epigastric discomfort:0.32), (INSULIN ASPART:Epigastric discomfort:0.02), (MELATONIN:Epigastric discomfort:0.16), (METHYLPREDNISOLONE SODIUM SUCCINATE:Epigastric discomfort:0.27), (METRONIDAZOLE:Epigastric discomfort:0.51), (NEBIVOLOL:Epigastric discomfort:0.02), (OLANZAPINE:Epigastric discomfort:0.18), (OXCARBAZEPINE:Epigastric discomfort:0.13), (PRAVASTATIN SODIUM:Epigastric discomfort:0.16), (SACUBITRILVALSARTAN:Epigastric discomfort:0.41), (TERIFLUNOMIDE:Epigastric discomfort:0.29), (TOCILIZUMAB:Epigastric discomfort:0.07), (WARFARIN SODIUM:Epigastric discomfort:0.11), (.ALPHA.1-PROTEINASE INHIBITOR HUMAN:Epilepsy:1.35), (ACECLOFENAC:Epilepsy:0.32), (ACETAMINOPHENAPRONALI DECAFFEINEPROPYPHENAZONE:Epilepsy:0.18), (ACETAMINOPHENCAFFEINE:Epilepsy: 0.55), (ACETAMINOPHENDIPHENHYDRAMINE HYDROCHLORIDE:Epilepsy:0.2), (ACETAMINOPHENOXYCODONEOXYCODONE HYDROCHLORIDE:Epilepsy:1.51), (ACETAMINOPHENPROPOXYPHENE HYDROCHLORIDE:Epilepsy:0.62), (ACETAMINOPHENPROPOXYPHENE NAPSYLATE:Epilepsy:0.69), (ADENINE:Epilepsy:1.26), (AGALSIDASE BETA:Epilepsy:1.18), (ALDESLEUKIN:Epilepsy:0.52), (ALISKIREN HEMIFUMARATE:Epilepsy:0.22), (ALUMINUM HYDROXIDEMAGNESIUM OXIDEOXETHAZAINE:Epilepsy:0.14), (AMLODIPINE BESYLATEATORVASTATIN CALCIUM:Epilepsy:1.03), (AMLODIPINE BESYLATEBENAZEPRIL HYDROCHLORIDE:Epilepsy:0.22), (AMLODIPINE BESYLATECANDESARTAN CILEXETIL:Epilepsy:0.52), (AMLODIPINE BESYLATEHYDROCHLOROTHIAZIDEOLMESARTAN MEDOXOMIL:Epilepsy:0.08), (AMLODIPINEVALSARTAN:Epilepsy:1.34), (AMMONIUM GLYCYRRHIZATECYSTEINE HYDROCHLORIDEGLYCINE:Epilepsy:0.18), (AMMONIUM LACTATE:Epilepsy:0.66), (AMOBARBITAL SODIUM:Epilepsy:0.46), (AMPHETAMINE SULFATE:Epilepsy:0), (ANAGLIPTIN:Epilepsy:0.22), (ANAGRELIDE:Epilepsy:0.35), (ANAG RELIDE HYDROCHLORIDE:Epilepsy:0.03), (ANTIHISTAMINES NOS:Epilepsy:0.67), (APRINDINE HYDROCHLORIDE:Epilepsy:0.08), (ARIPIPRAZOLE LAUROXIL:Epilepsy:0.95), (ASPIRINLANSOPRAZOLE:Epilepsy:0.23), (ATENOLOLCHLO RTHALIDONE:Epilepsy:0.22), (AZELASTINE HYDROCHLORIDEFLUTICASONE PROPIONATE:Epilepsy:0.03), (AZITHROMYCIN OR METHOTREXATE SODIUM:Epilepsy:0.85), (BALSALAZIDE DISODIUM:Epilepsy:0.06), (BELATACEPT:Epilepsy:0.35), (BERAPROST:Epilepsy:0.65), (BE TAHISTINE MESILATE:Epilepsy:1.46), (BETAMETHASONE DIPROPIONATECALCIPOTRIENECALCIPOTRIENE HYDRATE:Epilepsy:0.32), (BETAMETHASONE VALERATEGENTAMICIN SULFATE:Epilepsy:0.18), (BETAMIPRONPANIPENEM:Epilepsy:0.24), (BOCEPREVIR:Epilepsy: 0.57), (BREXPIPRAZOLE:Epilepsy:0.31), (BRIMONIDINE TARTRATE:Epilepsy:0.57), (BRIMONIDINE TARTRATETIMOLOL MALEATE:Epilepsy:0.24), (BUPRENORPHINENALOXONE:Epilepsy:0.64), (CALCITONIN SALMON:Epilepsy:1.26), (CALCIUM ACETATEMAGNESIUM ACETATEPOTASSIUM ACETATESODIUM ACETATESODIUM CHLORIDE:Epilepsy:0.17), (CALCIUM CARBONATEERGOCALCIFEROL:Epilepsy:0.02), (CALCIUM CARBONATEERGOCALCIFEROLRETINOL:Epilepsy:0.46), (CALCIUM PANTOTHENATECYANOCOBALAMINNIACINAMIDEPYRIDOXINE HYDROCHLORIDERIBOFLAVINTHIAMINE MONONITRATE:Epilepsy:0.06), (CALCIUMMAGNESIUMZINC:Epilepsy:0.27), (CANAGLI FLOZINMETFORMIN HYDROCHLORIDE:Epilepsy:0.92), (CARBOXYMETHYLCELLULOSE SODIUM:Epilepsy:0.09), (CARFILZOMIB:Epilepsy:1.49), (CARTEOLOL:Epilepsy:0.36), (CAR TEOLOL HYDROCHLORIDE:Epilepsy:0.3), (CARVEDILOL PHOSPHATE:Epilepsy:2.11), (CASPOFUNGIN:Epilepsy:1.16), (CEFADROXIL:Epilepsy:0.06), (CEFOTAXIMECEFOTAXIME SODIUM:Epilepsy:0.84), (CEFPODOXIME PROXETIL:Epilepsy:1.05), (CEFPROZIL:Epilepsy:0.02), (CELIPROLOL:Epilepsy:0.78), (CELL ULASEDIASTASELIPASE:Epilepsy:0.56), (CETIRIZINE HYDROCHLORIDEPSEUDOEPHEDRINE HYDROCHLORIDE:Epilepsy:0.3), (CHLORMADINONE ACETATE:Epilepsy:0.27), (CHLOROQUINE PHOSPHATE:Epilepsy:0.33), (CHLOROTHIAZIDE:Epilepsy:0.97), (CHLORPHENIRAMINE HYDROCODONE:Epilepsy:0.6), (CHLORPROTHIXENE:Epilepsy:0.79), (CHOLINE BITARTRATECYSTEINEINOSITOLMAMMAL LIVER:Epilepsy:0), (CHONDROITIN SULFATE (BOVINE)GLUCOSAMINE HYDROCHLORIDE:Epilepsy:0.68), (CHONDROITIN SULFATE (CHICKEN):Epilepsy:0.67), (CLOPERASTINE:Epilepsy:0.68), (CLOXACILLIN SODIUM:Epilepsy:0.36), (CODEINE PHOSPHATEGUAIFENESIN:Epilepsy:0.59), (CYANAMIDE:Epilepsy:0.17), (CYCLOPENTO LATE:Epilepsy:0.03), (DANAZOL:Epilepsy:1.19), (DARUNAVIRRITONAVIR:Epilepsy:0.11), (DECITABINE:Epilepsy:0.34), (DEFEROXAMINE MESYLATE:Epilepsy:0.07), (DEGARELIX:Epilepsy:0.76), (DESONIDE:Epilepsy:0.76), (DIEN OGEST:Epilepsy:0.04), (DILAZEP:Epilepsy:1.34), (DIMETHICONESILICON DIOXIDESIMETHICONE:Epilepsy:0.44), (DOCUSATE SODIUMSENNOSIDES A AND B:Epilepsy:0.09), (DOLASETRON MESYLATE:Epilepsy:0.18), (DORIPENEM:Epilepsy:0.19), (DORZOLAMIDE HYDROCHLORIDE:Epilepsy:0.5), (DRONABINOL:Epilepsy:1.63), (DRONEDARONE:Epilepsy: 0.66), (DURVALUMAB:Epilepsy:0.43), (DUTASTERIDETAMSULOSIN HYDROCHLORIDE:Epilepsy:0.67), (ECABET SODIUM:Epilepsy:0.33), (ELCATONIN:Epilepsy:0.02), (EMPAGLIFLOZINLINAGLIPTIN: Epilepsy:0.54), (ENALAPRIL MALEATEHYDROCHLOROTHIAZIDE:Epilepsy:1.09), (ENFUVIRTIDE:Epilepsy:0.04), (EP ALRESTAT:Epilepsy:0.27), (EPHEDRA SINICA STEMHERBALS:Epilepsy:0.49), (ERIBULIN:Epilepsy:0.43), (ERYTHROMYCINSULFISOX AZOLE:Epilepsy:0.23), (ESMOLOL HYDROCHLORIDE:Epilepsy:1.42), (ESTROGENS, CONJUGATEDMEDROXYPROGESTERONE ACETATE:Epilepsy:1.15), (ETHOTOIN:Epilepsy:0.03), (ETRETINATE:Epilepsy:1.89), (EXEN ATIDE:Epilepsy:0.14), (FAROPENEM SODIUM:Epilepsy:0.62), (FELBINAC:Epilepsy:0.05), (FERRIC OXYHYDROXIDE:Epilepsy:0.08), (FISH OIL\OMEGA-3 FATTY ACIDS:Epilepsy:0.32), (FLURAZEPAM HYDROCHLORIDE:Epilepsy:0.11), (FLUTAMIDE:Epilepsy:0.32), (FORMOTEROL FUMARATEMOMETASONE FUROATE:Epilepsy:1.19), (FOSFOMYCIN CALCIUM:Epilepsy:0.29), (FURSULTIAMINE:Epilepsy:0.73), (GABEXATE MESYLATE:Epilepsy:0.35), (GADOBENATE DIMEGLUMINE:Epilepsy:0.15), (GLYCOPYRROLATEINDACATEROL MALEATE:Epilepsy:0.82), (HYDROCHLOROTHIAZIDEQUINAPRIL HYDROCHLORIDE:Epilepsy:0.1), (HYDROCHLOROTHIAZIDETRIAMTERENE:Epilepsy: 0.29), (HYDROCODONE BITARTRATEIBUPROFEN:Epilepsy:0.42), (HYDROCORTISONE HEMISUCCINATE:Epilepsy:0.09), (IBRITUMOMAB TIUXETAN:Epilepsy:0.93), (IDARUCIZUMAB:Epilepsy:0.54), (IFENPRODIL TARTRATE:Epilepsy:0.13), (IGURATIMOD:Epilepsy:0.84), (ILOPERIDONE:Epilepsy:0.36), (I MIPENEM:Epilepsy:1.19), (INFLIXIMAB-DYYB:Epilepsy:1.04), (INOTUZUMAB OZOGAMICIN:Epilepsy:0.68), (IRINOTECAN HYDROCHLORIDE:Epilepsy:1.07), (ITRACONAZOLESODIUM CHLORIDE:Epilepsy:0.14), (IVACAFTOR:Epilepsy:0.16), (IVACAFTORLUMACAFTOR:Epilepsy: 0.04), (IXAZOMIB:Epilepsy:0.7), (KALLIDINOGENASE:Epilepsy:0.37), (KETOTIFEN: Epilepsy:0.09), (LACTOBACILLUS ACIDOPHILUSLACTOBACILLUS DELBRUECKII SUBSP. BULGARICUS:Epilepsy:0.15), (LANDIOLOL HYDROCHLORIDE:Epilepsy:0.59), (LANREOTIDE:Epilepsy:0.07), (LEVOBUPIVACAINE: Epilepsy:0.54), (LEVOCABASTINE HYDROCHLORIDE:Epilepsy:0.62), (LEVOLEUCOVORIN CALCIUM:Epilepsy:0.81), (LEVOMEFOLATE CALCIUMMETHYLCOBALAMINPYRIDOXAL PHOSPHATE ANHYDROUS:Epilepsy:0.19), (LEVOTHYROXINELIOTHYRONINE:Epilepsy:0.03), (LINSI DOMINE:Epilepsy:0.33), (LITHIUM SULFATE:Epilepsy:0.31), (LOMITAPIDE MESYLATE:Epilepsy:0.29), (LURASIDONE:Epilepsy:0.29), (MAGNESIUM ASPARTATE HYDROCHLORIDE:Epilepsy:0.91), (MAPROTILINE HYDROCHLORIDE:Epilepsy:0.27), (MECLIZINEMECLIZINE HYDROCHLORIDE:Epilepsy:1.14), (METHENOLONE ENANTHATE:Epilepsy:1.74), (METHYLENE BLUE:Epilepsy:0.87), (METRELEPTIN:Epilepsy:0.08), (MICAFUNGIN:Epilepsy:1.34), (MIFA MURTIDE:Epilepsy:0.01), (MILRINONE:Epilepsy:0.48), (MITIGLINIDE CALCIUM HYDRATEVOGLIBOSE:Epilepsy:0.3), (MOEXIPRIL HYDROCHLORIDE:Epilepsy:0.11), (MORPHINEMORPHINE HYDROCHLORIDEMORPHINE SULFATEMORPHINE TARTRATE:Epilepsy:0.85), (NAFAMOSTAT MESYLATE:Epilepsy:0.13), (NATEGLINIDE:Epilepsy:0.74), (NELARABINE:Epilepsy:0.18), (NIRAPARIB:Epilepsy:0.73), (NITROFURANTOINNITROFURANTOIN MONOHYDRATE:Epilepsy:0.65), (NUSINERSEN:Epilepsy:1.18), (OLAPARIB:Epilepsy:0.14), (ORPHENADRINE:Epilepsy:0.97), (OSPEMIFENE:Epilepsy:0.39), (OXYMORPHONE:Epilepsy: 0.72), (PAMIDRONATE DISODIUM:Epilepsy:1.21), (PANCURONIUM:Epilepsy:0.11), (PANITUMUMAB:Epilepsy:0.51), (PARICALCITOL:Epilepsy:0.63), (PATIROMER:Epilepsy:0.19), (PAZUFLOXACIN MESILATE:Epilepsy:0.35), (PEGASPARGASE:Epilepsy:0.07), (PEGINTERFERON ALFA-2A OR PEGINTERFERON ALFA-2B:Epilepsy:0.13), (PEGVISOMANT:Epilepsy:0.22), (PENICILLIN V BENZATHINE:Epilepsy:0.01), (PENTAZOCINE:Epilepsy:0.24), (PENTOSAN POLYSULFATE SODIUM:Epilepsy:1.07), (PENTOSTATIN:Epilepsy:0.59), (PEROSPIRONE:Epilepsy:1.47), (PH ENELZINE:Epilepsy:0.74), (PHENTERMINE HYDROCHLORIDE:Epilepsy:0), (PILOCARPINE HYDROCHLORIDE:Epilepsy:0.04), (PIMAVANSERIN TARTRATE:Epilepsy:0.42), (PIMOBENDAN:Epilepsy:1.56), (PIRFENIDONE:Epilepsy:0.28), (PIZOTYLINE:Epilepsy:0.09), (PNEUMOCOCCAL VACCINE, POLYVALENT 23:Epilepsy:0.83), (POLYETHYLENE GLYCOL 3350\POTASSIUM CHLORIDESODIUM BICARBONATESODIUM CHLORIDESODIUM SULFATE ANHYDROUS:Epilepsy:0.16), (POLYMYXIN B SULFATETRIMETHOPRIM SULFATE:Epilepsy:0), (POLYSTYRENE:Epilepsy:0.23), (POLYSTYRENE SULFONIC ACID:Epilepsy:0.28), (PORFIMER SODIUM:Epilepsy:0.23), (POTASSIUM IODIDE:Epilepsy:0.6), (POVIDONE:Epilepsy:0.5), (PRAZOSIN HYDROCHLORIDE:Epilepsy:0.88), (PREDNISOLONE SODIUM PHOSPHATE:Epilepsy:1.79), (PROMAZINE:Epilepsy:0.4), (QUINACRINE:Epilepsy:0.35), (RA DIATION THERAPY:Epilepsy:1.56), (RAMUCIRUMAB:Epilepsy:0.81), (RASAGILINE:Epilepsy:0.14), (RISEDRONIC ACID:Epilepsy:0.13), (ROFECOXIB:Epilepsy:0.32), (ROFLUMILAST:Epilepsy:1.72), (SAPRO PTERIN DIHYDROCHLORIDE:Epilepsy:0.5), (SARPOGRELATE HYDROCHLORIDE:Epilepsy:0.91), (SELEXIPAG:Epilepsy:0.53), (SENNOSIDE ASENNOSIDE BSENNOSIDES:Epilepsy:0.19), (SENNOSIDE B:Epilepsy:0.21), (SERRAPEPTASE:Epilepsy:0.28), (SEVELAMER CARBONATE:Epilepsy:0.34), (SEVELAMER HYDROCHLORIDE:Epilepsy:1.3), (SILICON DIOXIDE:Epilepsy:0.16), (SILVER SULFADIAZINE:Epilepsy:0.75), (SITAFLOXACIN:Epilepsy:0.27), (SIVELESTAT SODIUM:Epilepsy:0.31), (SODIUM GUALENATE MONOHYDRATE:Epilepsy:0.11), (SOFALCONE:Epilepsy:0.18), (SORBITOL:Epilepsy:0.14), (STAVUDINE:Epilepsy:0.18), (STREPTOMYCIN SULFATE:Epilepsy:0.53), (STREPTOMYCINSTREPTOMYCIN SULFATE:Epilepsy:1.07), (SULINDAC:Epilepsy:0.33), (SUNITINIB:Epilepsy:0.02), (SUPLAT AST TOSILATE:Epilepsy:1.32), (TANDOSPIRONE CITRATE:Epilepsy:0.23), (TEDUGLUTIDEWATER:Epilepsy:0.36), (TESTOSTERONE UNDECANOATE:Epilepsy:0.89), (TETRACAINE HYDROCHLORIDE:Epilepsy:0.18), (THEOPHYLLINE:Epilepsy:0.46), (THYROID, PORCINETHYROID, UNSPECIFIED:Epilepsy:0), (TIBOLONE:Epilepsy:0.04), (TIMOLOL MALEATE:Epilepsy:0.93), (TIMOLOL MALEATETRAVOPROST:Epilepsy:0.4), (TOFISOPAM:Epilepsy:0.48), (TRABECTEDIN:Epilepsy: 0.08), (TRIFLUOPERAZINETRIFLUOPERAZINE HYDROCHLORIDE:Epilepsy:0.16), (TRIMETAZIDINE:Epilepsy:0.04), (TROSPIUM:Epilepsy: 0.14), (TROXIPIDE:Epilepsy:0.2), (UMECLIDINIUM BROMIDE:Epilepsy:1.01), (UREA:Epilepsy:0.26), (UROKINASE:Epilepsy:0.3), (VARDENAFI L HYDROCHLORIDE TRIHYDRATE:Epilepsy:0.34), (VONOPRAZAN:Epilepsy:0.26), (ZALTOPROFEN:Epilepsy:0.28), (ZANAMIVIR:Epilepsy:0.24), (ZICONOTIDE ACETATE:Epilepsy:0.48), (ZILEUTON:Epilepsy:0.44), (ZUCLOPENTHIXOL:Epilepsy:1.26), (ZUCLOPENTHIXOL DECANOATE:Epilepsy:0.47), (ANASTROZOLE:Epistaxis:0.14), (BORTEZOMIB:Epistaxis:0.33), (CALCIUM CARBONATECHOLECALCIFEROL:Epistaxis:0.31), (CARVEDILOL:Epistaxis:0.18), (CERT OLIZUMAB PEGOL:Epistaxis:0.11), (DEXLANSOPRAZOLE:Epistaxis:0.09), (DILTIAZEM HYDROCHLORIDE:Epistaxis:0.66), (EVEROLIMUS:Epistaxis:0.1), (HYDROCHLOROTHIA ZIDELISINOPRIL:Epistaxis:0.11), (IMATINIB MESYLATE:Epistaxis:0.06), (MELOXICAM:Epistaxis:0.12), (METRONIDAZOLE:Epistaxis:0.85), (NIFEDIPINE:Epistaxis:0.27), (NILOTINIB:Epistaxis:0.5), (POTASSIUM:Epistaxis:0.18), (P RAVASTATIN SODIUM:Epistaxis:0.22), (PROMETHAZINEPROMETHAZINE HYDROCHLORIDE:Epistaxis:0), (SACUBITRILVALSARTAN:Epistaxis:0.67), (TERIFLUNO MIDE:Epistaxis:0.18), (TIOTROPIUM BROMIDE MONOHYDRATE:Epistaxis:0.11), (VENLAFAXINE HYDROCHLORIDE:Epistaxis:0.07), (VINCRISTINE SULFATE:Epistaxis:0.07), (WARFARIN SODIUM:Epistaxis:0.69), (.ALPHA.1-PROTEINASE INHIBITOR HUMAN:Erectile dysfunction:0.13), (ABACAVIR:Erectile dysfunction:0.05), (ACENOCOUMAROL:Erectile dysfunction:0.52), (ACETAMINOPHENTRAMADOL:Erectile dysfunction:0.22), (ACITRETIN:Erectile dysfunction:0.14), (ADO-TRASTUZUMAB EMTANSINE:Erectile dysfunction:0.17), (AGALSIDASE BETA:Erectile dysfunction:0.34), (ALBUTEROLIPRATROPIUM:Erectile dysfunction:0.02), (ALENDRONIC ACID:Erectile dysfunction:0.53), (ALFACALCIDOL:Erectile dysfunction:0.72), (AMISULPRIDE:Erectile dysfunction:0.39), (AMPHOTERICIN B:Erectile dysfunction:0.05), (ANTIHISTAMINES NOS:Erectile dysfunction:0.17), (APREPITANT:Erectile dysfunction:0.34), (ARIPIPRAZOLE LAUROXIL:Erectile dysfunction:0.46), (BENZTROPINE MESYLATE:Erectile dysfunction:0.39), (BETAMETHASONECALCIPOTRIENE:Erectile dysfunction:0.15), (BUPRENORPHINE HYDROCHLORIDE:Erectile dysfunction:0.92), (CALCITONIN:Erectile dysfunction:0.42), (CALCITONIN SALMON:Erectile dysfunction:0.51), (CALCIUMMAGNESIUM:Erectile dysfunction:0.56), (CARVEDILOL PHOSPHATE:Erectile dysfunction:1.81), (CEFUROXIME AXETIL:Erectile dysfunction:0.02), (CETIRIZINE HYDROCHLORIDEPSEUDOEPHEDRINE HYDROCHLORIDE:Erectile dysfunction:0.63), (CEVIMELINE HYDROCHLORIDE:Erectile dysfunction:0.2), (CHLORDIAZEPOXIDE HYDROCHLORIDECLIDINIUM BROMIDE:Erectile dysfunction:0.28), (CILASTATIN SODIUMIMIPENEM:Erectile dysfunction:0.2), (CIMETIDINE:Erectile dysfunction:1.35), (CLIKSTAR:Erectile dysfunction:0.2), (DACLATASVIR:Erectile dysfunction:0.19), (DALTEPARIN:Erectile dysfunction:0.48), (DANAZOL:Erectile dysfunction:0.12), (DEFLAZACORT:Erectile dysfunction:0.53), (DEXTROMETHORPHAN HYDROBROMIDEGUAIFENESIN:Erectile dysfunction:0.11), (DIMETHYL SULFOXIDE:Erectile dysfunction:0.18), (DIPYRONE:Erectile dysfunction:0.51), (DOLASETRON MESYLATE:Erectile dysfunction:0.03), (EDOXABAN TOSYLATE:Erectile dysfunction:0.91), (EMPAGLIFLOZINMETFORMIN HYDROCHLORIDE:Erectile dysfunction:0.25), (EPIRUBICIN:Erectile dysfunction:0.06), (ESTROGENS, CONJUGATED:Erectile dysfunction:1.54), (ESTROGENS, CONJUGATEDMEDROXYPROGESTERONE ACETATE:Erectile dysfunction:0.72), (ESTRONESUS SCROFA OVARY:Erectile dysfunction:0.35), (EXEMESTANE:Erectile dysfunction:0.1), (FERROUS GLUCONATE:Erectile dysfunction:0.29), (FLUTICASONE FUROATESALMETEROL XINAFOATE:Erectile dysfunction:0.65), (FORMOTEROL:Erectile dysfunction:0.12), (FORMOTEROL FUMARATEMOMETASONE FUROATE:Erectile dysfunction:0.2), (FUSIDIC ACID:Erectile dysfunction:0.54), (GLIMEPIRIDEROSIGLITAZONE MALEATE:Erectile dysfunction:0), (GUANFACINE HYDROCHLORIDE:Erectile dysfunction:0.82), (HYDROCODONE BITARTRATEIBUPROFEN:Erectile dysfunction:0.52), (ILOPROST:Erectile dysfunction:0.35), (IMIQUIMOD:Erectile dysfunction:0.02), (INDAPAMIDEPERINDOPRIL:Erectile dysfunction:0.73), (INTERFERON ALFA-2A:Erectile dysfunction:0.37), (IODINE:Erectile dysfunction:0.26), (IRINOTECAN:Erectile dysfunction:0.37), (IRINOTECAN HYDROCHLORIDE:Erectile dysfunction:0.14), (LABETALOL HYDROCHLORIDE:Erectile dysfunction:0.02), (LAPATINIB DITOSYLATE:Erectile dysfunction:0.06), (LERCANIDIPINE HYDROCHLORIDE:Erectile dysfunction:0.59), (LEVALBUTEROL HYDROCHLORIDE:Erectile dysfunction:1.94), (LEVOMILNACIPRAN HYDROCHLORIDE:Erectile dysfunction:0.37), (LIDOCAINE HYDROCHLORIDE:Erectile dysfunction:1.48), (MEPOLIZUMAB:Erectile dysfunction:0.11), (MESNA:Erectile dysfunction:0.07), (METAMIZOLE:Erectile dysfunction:0.05), (METFORMIN HYDROCHLORIDEVILDAGLIPTIN:Erectile dysfunction:0.44), (METHIMAZOLE:Erectile dysfunction:0.34), (METHYLPREDNISOLONE ACETATE:Erectile dysfunction:1.5), (METRONIDAZOLEMETRONIDAZOLE HYDROCHLORIDE:Erectile dysfunction:1.24), (MOXIFLOXACIN:Erectile dysfunction:0.47), (NALOXONE HYDROCHLORIDEOXYCODONE HYDROCHLORIDE:Erectile dysfunction:0.98), (NINTEDANIB:Erectile dysfunction:0.71), (NIRAPARIB:Erectile dysfunction:0.35), (NITROFURANTOINNITROFURANTOIN MONOHYDRATE:Erectile dysfunction:0.43), (OXYBUTYNINOXYBUTYNIN CHLORIDE:Erectile dysfunction:0.45), (PALBOCICLIB:Erectile dysfunction:0.01), (PANTOPRAZOLE MAGNESIUM:Erectile dysfunction:1.53), (PARATHYROID HORMONE:Erectile dysfunction:0.04), (PEGINTERFERON BETA-1A:Erectile dysfunction:0.98), (PEMETREXED DISODIUM:Erectile dysfunction:0.65), (PHYTONADIONE:Erectile dysfunction:0.22), (PRAZOSIN HYDROCHLORIDE:Erectile dysfunction:0.43), (PROGESTERONE:Erectile dysfunction:0.09), (PROPAFENONE HYDROCHLORIDE:Erectile dysfunction:0.67), (PROTRIPTYLINE HYDROCHLORIDE:Erectile dysfunction:0.22), (PYRIDOSTIGMINE BROMIDE:Erectile dysfunction:0.21), (RADIATION THERAPY:Erectile dysfunction:0.25), (RALOXIFENE HYDROCHLORIDE:Erectile dysfunction:1.38), (REGORAFENIB:Erectile dysfunction:0.48), (RIVASTIGMINE:Erectile dysfunction:0.68), (RIVASTIGMINE TARTRATE:Erectile dysfunction:0.75), (RIZATRIPTAN:Erectile dysfunction:0.28), (ROFECOXIB:Erectile dysfunction:0.03), (SALMETEROL:Erectile dysfunction:0.35), (SCOPOLAMINE:Erectile dysfunction:0.96), (SEVELAMER CARBONATE:Erectile dysfunction:0.17), (TAPENTADOL HYDROCHLORIDE:Erectile dysfunction:0.18), (TETRACYCLINE:Erectile dysfunction:1.57), (TROSPIUM CHLORIDE:Erectile dysfunction:0.24), (UMECLIDINIUM BROMIDEVILANTEROL TRIFENATATE:Erectile dysfunction:1.33), (VALGANCICLOVIR HYDROCHLORIDE:Erectile dysfunction:0.79), (ZAFIRLUKAST:Erectile dysfunction:0.61), (ZILEUTON:Erectile dysfunction:0.22), (ZOLMITRIPTAN:Erectile dysfunction:0.36), (NAPROXEN:Erosive oesophagitis:0.34), (ANASTROZOLE:Erysipelas:0.15), (CALCIUM CARBONATECHOLECALCIFEROL:Erysipelas:0.29), (CELECOXIB:Erysipelas:0.42), (CITA LOPRAM HYDROBROMIDE:Erysipelas:0.24), (DASATINIB:Erysipelas:0.1), (DOXAZOSINDOXAZOS IN MESYLATE:Erysipelas:0.05), (FERROUS SULFATEFERROUS SULFATE, DRIED:Erysipelas:0.35), (FISH OIL:Erysipelas:0.16), (FLECAINIDE:Erysipelas:0.15), (IBRUTINIB:Erysipelas:0.71), (ISOSOR BIDE MONONITRATE:Erysipelas:0.16), (LEDIPASVIRSOFOSBUVIR:Erysipelas:0.16), (LOVAST ATIN:Erysipelas:0.25), (METAMIZOLE:Erysipelas:0.1), (NILOTINIB:Erysipelas:0.33), (OMEP RAZOLE MAGNESIUM:Erysipelas:0.21), (RANOLAZINE:Erysipelas:0.06), (SERTRALINE HYDROCHLORIDE:Erysipelas:0.1), (AMIODARONE HYDROCHLORIDE:Erythema:0.43), (AMPHETAMINE ASPARTATEAMPHETAMINE SULFATEDEXTROAMPHETAMINE SACCHARATEDEXTROAMPHETAMINE SULFATE:Erythema:0.13), (BISACODYL OR DOCUSATE SODIUM:Erythema:0.26), (BUMETANIDE:Erythema:0.56), (CANAKINUMAB:Erythema:0.09), (CANDESARTAN CILEXETIL:Erythema:0.77), (CANDESARTAN CILEXETILHYDROCHLOROTHIAZIDE:Erythema:0.38), (CHOLESTYRAMINE:Erythema:00.38), (CILASTATIN SODIUMIMIPENEM:Erythema:0.31), (CLOZAPINE:Erythema:0.15), (CODEINE:Erythema:0), (DASATINIB:Erythema:1.03), (DOCUSATE:Erythema:0.35), (ELETRIPTAN HYDROBROMIDE:Erythema:0.07), (ESZOPICLONE:Erythema:0.78), (ETORICOXIB:Erythema: 0.11), (EVOLOCUMAB:Erythema:0.76), (FINGOLIMOD HYDROCHLORIDE:Erythema:0.32), (FLUTICASONE FUROATEVILANTEROL TRIFENATATE:Erythema:0.07), (HYDRALAZINE HYDROCHLORIDE:Erythema:0.04), (HYDROCHLOROTHIAZIDEIRBESARTAN:Erythema: 0.03), (HYDROCHLOROTHIAZIDEVALSARTAN:Erythema:0.1), (HYDROMORPHONE: Erythema:0.38), (HYDROMORPHONE HYDROCHLORIDE:Erythema:0.46), (HYDROXYCHLOROQUINE DIPHOSPHATE:Erythema:0.16), (IRBESARTAN:Erythema:0.19), (ISOSORBIDE:Erythema:0.05), (LABETALOLLABETALOL HYDROCHLORIDE:Erythema:0.07), (LEDIPASVIRSOFOSBUVIR:Erythema:0.26), (METHA DONE HYDROCHLORIDE:Erythema:0.04), (METHYLPREDNISOLONE ACETATE:Erythema:0.31), (MOXIFLOXACIN:Erythema:0.01), (MOXIFLOXACIN HYDROCHLORIDE:Erythema:0.69), (NEBIVOLOL HYDROCHLORIDE:Erythema:0.01), (PANTOPRAZOLE MAGNESIUM:Erythema:0.1), (PAZOPANIB HYDROCHLORIDE:Erythema:0.15), (PHENOBARBITAL:Erythema:0.66), (PITAVASTATIN CALCIUM:Erythema:0.1), (PRIMIDONE:Erythema:0.64), (PROMETHAZINEPROMETHAZI NE HYDROCHLORIDE:Erythema:0.35), (PYRIDOXINE:Erythema:0.03), (QUETIAPINE:Erythema: 0.08), (RANOLAZINE:Erythema:0.32), (SACUBITRILVALSARTAN:Erythema:0.92), (SOTA LOL:Erythema:0.22), (SULFAMETHOXAZOLE:Erythema:0.18), (TEMAZEPAM:Erythema:0.37), (THALIDOMIDE:Erythema:0.97), (VARENICLINE TARTRATE:Erythema:0.23), (VITAMIN B:Erythema:0.3), (AMIODARONE:Escherichia infection:0.11), (BUMETANIDE:Escherichia infection:0.15), (DESFLURANE:Escherichia infection:0.12), (DEXLANSOPRAZOLE:Escherichia infection:0.13), (DILTIAZEM HYDROCHLORIDE:Escherichia infection:0.29), (FLUTICASONE PROPIONATE:Escherichia infection:0.08), (IBRUTINIB:Escherichia infection:0.12), (LERCANIDIPINE:Escherichia infection:0.19), (LEVOTHYROXINE:Escherichia infection:0.13), (THALIDOMIDE:Escherichia infection:0.01), (ACETAMINOPHENOXYCODONEOXYCODONE HYDROCHLORIDE:Essential hypertension:0.39), (ACETAMINOPHENPROPOXYPHENE HYDROCHLORIDE:Essential hypertension:0.13), (ACETAMINOPHENTRAMADOL:Essential hypertension:0.19), (ADO-TRASTUZUMAB EMTANSINE:Essential hypertension:0.44), (AGALSIDASE BETA:Essential hypertension:0.23), (ALBIGLUTIDE:Essential hypertension:0.14), (ALCOHOL:Essential hypertension:0.33), (ALFACALCIDOL:Essential hypertension:0.87), (ALISKIREN HEMIFUMARATE:Essential hypertension:0.07), (ALUMINUM HYDROXIDEDIMETHICONEMAGNESIUM HYDROXIDE:Essential hypertension:0.22), (AMPHOTERICIN B:Essential hypertension:0.09),(ARMODAFINIL:Essential hypertension:0.65), (AZELASTINE:Essential hypertension:0.04), (AZOSEMIDE:Essential hypertension:0.11), (BETAMETHASONE DIPROPIONATE:Essential hypertension:0.32), (BICALUTAMIDE:Essential hypertension:0.86), (BIFIDOBACTERIUM LONGUM SUBSP. INFANTIS:Essential hypertension:0.05), (BUPRENORPHINE HYDROCHLORIDENALOXONE HYDROCHLORIDE:Essential hypertension:0.45), (BUTYLSCOPOLAMINE BROMIDE:Essential hypertension:0.01), (CALCITONIN:Essential hypertension:0.01), (CEFOTAXIME SODIUM:Essential hypertension:0.35), (CHLORPROMAZINE:Essential hypertension:0.04), (CHONDROITIN SULFATE (BOVINE)GLUCOSAMINE HYDROCHLORIDE:Essential hypertension:0.22), (CIMETIDINE:Essential hypertension:0.74), (CLINDAMYCIN:Essential hypertension:0.71), (CORTICOTROPIN:Essential hypertension:0.16), (DABIGATRAN:Essential hypertension:1.38), (DACLIZUMAB:Essential hypertension:0.36), (DALTEPARIN:Essential hypertension:0.55), (DALTEPARIN SODIUM:Essential hypertension:0.71), (DANAZOL:Essential hypertension:0.49), (DASABUVIROMBITASVIRPARITAPREVIRRITONAVIR:Essential hypertension:0.08), (DASATINIB:Essential hypertension:2.12), (DEXAMETHASONE ACETATE:Essential hypertension:0.22), (DIMENHYDRINATE:Essential hypertension:0.47), (DRONABINOL:Essential hypertension:0.62), (DUTASTERIDETAMSULOSIN HYDROCHLORIDE:Essential hypertension:0.33), (ECONAZOLE:Essential hypertension:0.6), (ELETRIPTAN HYDROBROMIDE:Essential hypertension:0.84), (EMPAGLIFLOZINMETFORMIN HYDROCHLORIDE:Essential hypertension:0.27), (EPIRUBICIN:Essential hypertension:0.75), (EPLERENONE:Essential hypertension:1.14), (FENOTEROL HYDROBROMIDEIPRATROPIUM BROMIDE:Essential hypertension:0.41), (FLUDROCORTISONE ACETATE:Essential hypertension:0.44), (FLUOCINONIDE:Essential hypertension:0.17), (FLUVOXAMINE MALEATE:Essential hypertension:0.07), (FONDAPARINUX:Essential hypertension:0.04), (FORMOTEROL FUMARATEMOMETASONE FUROATE:Essential hypertension:0.35), (FULVESTRANT:Essential hypertension:0.19), (FUSIDIC ACID:Essential hypertension:0.41), (GLIMEPIRIDEROSIGLITAZONE MALEATE:Essential hypertension:0.2), (GLYCOPYRROLATEINDACATEROL MALEATE:Essential hypertension:0), (HERBALSTURMERIC:Essential hypertension:0.15), (HYDROXYCHLOROQUINE DIPHOSPHATE:Essential hypertension:0.48), (HYOSCYAMINE:Essential hypertension:0.44), (IDARUCIZUMAB:Essential hypertension:0.15), (IMIPRAMINE HYDROCHLORIDE:Essential hypertension:0.04), (IRINOTECAN HYDROCHLORIDE:Essential hypertension:0.33), (LACTASE:Essential hypertension:0.07), (LACTOBACILLUS RHAMNOSUS:Essential hypertension:0.04), (LEUCOVORIN CALCIUM:Essential hypertension:0.92), (LEVALBUTEROL HYDROCHLORIDE:Essential hypertension:1.11), (LIDOCAINEPRILOCAINE:Essential hypertension:0.47), (MECLIZINEMECLIZINE HYDROCHLORIDE:Essential hypertension:0.16), (MEPOLIZUMAB:Essential hypertension:0.38), (METHENAMINE HIPPURATE:Essential hypertension:0.5), (METHYLPHENIDATE HYDROCHLORIDE:Essential hypertension:0.63), (MIDODRINE HYDROCHLORIDE:Essential hypertension:0.07), (MINOCYCLINE HYDROCHLORIDE:Essential hypertension:0.02), (MINOCYCLINEMINOCYCLINE HYDROCHLORIDE:Essential hypertension:0.68), (NALOXONE HYDROCHLORIDEOXYCODONE HYDROCHLORIDE:Essential hypertension:1.44), (NATEGLINIDE:Essential hypertension:0.66), (NIRAPARIB:Essential hypertension:0.06), (PANITUMUMAB:Essential hypertension:0.06), (PANTOPRAZOLE MAGNESIUM:Essential hypertension:1.86), (PARICALCITOL:Essential hypertension:0.58), (PEGINTERFERON BETA-1A:Essential hypertension:0.58), (PEMETREXED:Essential hypertension:0.21), (PEMETREXED DISODIUM:Essential hypertension:0.77), (PHENYTOIN SODIUM:Essential hypertension:0.04), (PILSICAINIDE HYDROCHLORIDE:Essential hypertension:0.04), (PIROXICAM:Essential hypertension:0.51), (PLANTAGO SEED:Essential hypertension:0.47), (PSYLLIUM HUSK:Essential hypertension:0.2), (PYRIDOSTIGMINE BROMIDE:Essential hypertension:0.13), (RIVASTIGMINE TARTRATE:Essential hypertension:0.3), (RIZATRIPTAN BENZOATE:Essential hypertension:0.28), (ROPINIROLE:Essential hypertension:0.33), (ROSIGLITAZONE MALEATE:Essential hypertension:0.72), (SACCHAROMYCES CEREVISIAE:Essential hypertension:0.25), (SALMETEROL XINAFOATE:Essential hypertension:0.14), (SEVELAMER HYDROCHLORIDE:Essential hypertension:0.17), (SODIUM OXYBATE:Essential hypertension:0.81), (SODIUM PHOSPHATE, DIBASICSODIUM PHOSPHATE, MONOBASIC:Essential hypertension:0.13), (SUMATRIPTAN:Essential hypertension:0.38), (TERBINAFINE HYDROCHLORIDE:Essential hypertension:0.34), (TERBUTALINE SULFATE:Essential hypertension:0.74), (TICLOPIDINE:Essential hypertension:0.04), (TINZAPARIN:Essential hypertension:0.26), (TINZAPARIN SODIUM:Essential hypertension:0.55), (TROSPIUM CHLORIDE:Essential hypertension:0.18), (UMECLIDINIUM BROMIDE:Essential hypertension:0.24), (UMECLIDINIUM BROMIDEVILANTEROL TRIFENATATE:Essential hypertension:1.21), (VILAZODONE HYDROCHLORIDE:Essential hypertension:0.04), (VINCRISTINE SULFATE:Essential hypertension:0.22), (VITAMIN A:Essential hypertension:0.3), (ZINC:Essential hypertension:0.43), (ACENOCOUMAROL:Essential thrombocythaemia:0.02), (ANASTROZOLE:Essential thrombocythaemia:0.27), (BISACODYL OR DOCUSATE SODIUM:Essential thrombocythaemia:0.03), (BORTEZOMIB:Essential thrombocythaemia:0.2), (BROMAZEPAM:Essential thrombocythaemia:0.1), (BUDESONIDE:Essential thrombocythaemia:0.75), (CALCITRIOL:Essential thrombocythaemia:0.06), (CERTOLIZUMAB PEGOL:Essential thrombocythaemia:0.68), (CIPROFLOXACIN:Essential thrombocythaemia:0.11), (CORTISONEHYDROCORTISONE:Essential thrombocythaemia:0.13), (CYCLOBENZAPRINE:Essential thrombocythaemia:0.09), (DEXLANSOPRAZOLE:Essential thrombocythaemia:0.47), (DIPHENHYDRAMINE:Essential thrombocythaemia:0.17), (EDOXABAN TOSYLATE:Essential thrombocythaemia:0.23), (EPINEPHRINE:Essential thrombocythaemia:0.1), (EVEROLIMUS:Essential thrombocythaemia:0.53), (EVOLOCUMAB:Essential thrombocythaemia:0.53), (EZETIMIBE:Essential thrombocythaemia:0.52), (GLIPIZIDE:Essential thrombocythaemia:0.3), (IBRUTINIB:Essential thrombocythaemia:1.14), (ILOPROST:Essential thrombocythaemia:0.3), (IMATINIB MESYLATE:Essential thrombocythaemia:0.42), (INDAPAMIDEPERINDOPRIL:Essential thrombocythaemia:0.27), (INSULIN HUMAN:Essential thrombocythaemia:0.23), (IRBESARTAN:Essential thrombocythaemia:0.31), (ISOSORBIDE:Essential thrombocythaemia:0.2), (LEDIPASVIRSOFOSBUVIR:Essential thrombocythaemia:0.15), (NILOTINIB:Essential thrombocythaemia:0.61), (PAROXETINEPAROXETINE HYDROCHLORIDE:Essential thrombocythaemia:0), (POLYETHYLENE GLYCOLS:Essential thrombocythaemia:0.4), (PRAMIPEXOLE DIHYDROCHLORIDE:Essential thrombocythaemia:0.1), (RANOLAZINE:Essential thrombocythaemia:0.64), (SECUKINUMAB:Essential thrombocythaemia:0.15), (TERIFLUNOMIDE:Essential thrombocythaemia:0.4), (TOFACITINIB CITRATE:Essential thrombocythaemia:0.05), (TOPIRAMATE:Essential thrombocythaemia:0.28), (VITAMIN B:Essential thrombocythaemia:0.11), (AMIODARONE:Essential tremor:0.39), (AMIODARONE HYDROCHLORIDE:Essential tremor:0.12), (AMOXICILLIN:Essential tremor:0.32), (BENAZEPRIL HYDROCHLORIDE:Essential tremor:0.01), (BISACODYL OR DOCUSATE SODIUM:Essential tremor:0.19), (BUPRENORPHINE HYDROCHLORIDENALOXONE HYDROCHLORIDE:Essential tremor:0.14), (CALCITRIOL:Essential tremor:0.04), (CANDESARTAN CILEXETIL:Essential tremor:0.24), (CARVEDILOL PHOSPHATE:Essential tremor:0.23), (CEPHALEXIN:Essential tremor:0.33), (CLINDAMYCINCLINDAMYCIN PHOSPHATE:Essential tremor:0.08), (CLOBAZAM:Essential tremor:0.02), (CLOZAPINE:Essential tremor:0.26), (CORTISONEHYDROCORTISONE:Essential tremor:0.29), (DACLIZUMAB:Essential tremor:0.22), (DASATINIB:Essential tremor:0.39), (DICLOFENAC:Essential tremor:0.63), (DIPHENHYDRAMINE:Essential tremor:0.89), (EVEROLIMUS:Essential tremor:0.88), (EZETIMIBESIMVASTATIN:Essential tremor:0.02), (FLUOXETINEFLUOXETINE HYDROCHLORIDE:Essential tremor:0.16), (FLUTICASONE:Essential tremor:0.11), (FLUTICASONE FUROATEVILANTEROL TRIFENATATE:Essential tremor:0.03), (HEPARIN SODIUM:Essential tremor:0.2), (HYDROXYZINE PAMOATE:Essential tremor:0.55), (IBRUTINIB:Essential tremor:0.76), (INDOMETHACIN:Essential tremor:0.09), (LEDIPASVIRSOFOSBUVIR:Essential tremor:0.25), (METHYLPREDNISOLONE ACETATE:Essential tremor:0.15), (METRONIDAZOLE:Essential tremor:0.79), (NEBIVOLOL:Essential tremor:0.01), (NILOTINIB:Essential tremor:0.1), (OMEGA-3-ACID ETHYL ESTERS:Essential tremor:0.1), (OXCARBAZEPINE:Essential tremor:1.64), (OXYGEN:Essential tremor:0.4), (PENICILLIN:Essential tremor:0.13), (POLYETHYLENE GLYCOLS:Essential tremor:0.49), (PROMETHAZINEPROMETHAZINE HYDROCHLORIDE:Essential tremor:0.63), (RALOXIFENE HYDROCHLORIDE:Essential tremor:0.26), (SACUBITRILVALSARTAN:Essential tremor:0.31), (SCOPOLAMINE:Essential tremor:0), (SOTALOL:Essential tremor:0.02), (TERIFLUNOMIDE:Essential tremor:0.94), (UMECLIDINIUM BROMIDEVILANTEROL TRIFENATATE:Essential tremor:0.13), (VARENICLINE TARTRATE:Essential tremor:1.43), (VILAZODONE HYDROCHLORIDE:Essential tremor:0.21), (VITAMIN B:Essential tremor:0.44), (ZINC:Essential tremor:0.07), (AMIKACIN SULFATE:Evidence based treatment:0.02), (BLINATUMOMAB:Evidence based treatment:0.46), (CEPHALEXIN:Evidence based treatment:0.19), (FLUTICASONE PROPIONATE:Evidence based treatment:0.02), (IBRITUMOMAB TIUXETAN:Evidence based treatment:0.05), (NEBIVOLOL:Evidence based treatment:0.06), (OXCARBAZEPINE:Evidence based treatment:0.65), (THALIDOMIDE:Evidence based treatment:0.61), (ZONISAMIDE:Evidence based treatment:0.1), (AMIODARONE HYDROCHLORIDE:Ex-tobacco user:0.16), (AMISULPRIDE:Ex-tobacco user:0.15), (AMITRIPTYLINE HYDROCHLORIDE:Ex-tobacco user:0.06), (BORTEZOMIB:Ex-tobacco user:0.19), (CALCIUM CITRATE:Ex-tobacco user:0.03), (CARFILZOMIB:Ex-tobacco user:0.18), (CARVEDILOL PHOSPHATE:Ex-tobacco user:0.04), (CHOLESTYRAMINE:Ex-tobacco user:0.29), (CIPROFLOXACIN HYDROCHLORIDE:Ex-tobacco user:0.36), (CLOTRIMAZOLE:Ex-tobacco user:0.08), (CORTISONEHYDROCORTISONE:Ex-tobacco user:0.41), (DALFAMPRIDINE:Ex-tobacco user:1.03), (DASATINIB:Ex-tobacco user:0.1), (DEVICE:Ex-tobacco user:0.14), (DUTASTERIDE:Ex-tobacco user:0.16), (ELETRIPTAN HYDROBROMIDE:Ex-tobacco user:0.21), (ENALAPRIL:Ex-tobacco user:1.31), (EPINEPHRINE:Ex-tobacco user:0.48), (ESZOPICLONE:Ex-tobacco user:0.62), (EVEROLIMUS:Ex-tobacco user:0.13), (EVOLOCUMAB:Ex-tobacco user:0.78), (FERROUS SULFATE:Ex-tobacco user:0.94), (FESOTERODINE FUMARATE:Ex-tobacco user:0.07), (FLECAINIDE:Ex-tobacco user:0.18), (IBRUTINIB:Ex-tobacco user:0.82), (ILOPROST:Ex-tobacco user:0.15), (IMIPRAMINE HYDROCHLORIDE:Ex-tobacco user:0.04), (INDOMETHACIN:Ex-tobacco user:0.23), (ISOSORBIDE:Ex-tobacco user:0.19), (ISOSORBIDE MONONITRATE:Ex-tobacco user:0.59), (LEVALBUTEROL HYDROCHLORIDE:Ex-tobacco user:0.27), (LIDOCAINE HYDROCHLORIDE:Ex-tobacco user:0.02), (METHYLPREDNISOLONE ACETATE:Ex-tobacco user:0.28), (NEBIVOLOL:Ex-tobacco user:0.15), (NILOTINIB:Ex-tobacco user:0.65), (OXYBUTYNIN CHLORIDE:Ex-tobacco user:0.07), (PANTOPRAZOLE MAGNESIUM:Ex-tobacco user:0.24), (PENICILLIN:Ex-tobacco user:0.18), (PHENOBARBITAL:Ex-tobacco user:0.08), (POLYETHYLENE GLYCOL 3350:Ex-tobacco user:0.6), (PROGESTERONE:Ex-tobacco user:0.15), (QUETIAPINE:Ex-tobacco user:0.82), (RISEDRONATE SODIUM:Ex-tobacco user:0.32), (RIZATRIPTAN BENZOATE:Ex-tobacco user:0.14), (ROFLUMILAST:Ex-tobacco user:0.03), (SACUBITRILVALSARTAN:Ex-tobacco user:0.6), (SECUKINUMAB:Ex-tobacco user:0.61), (SUMATRIPTANSUMATRIPTAN SUCCINATE:Ex-tobacco user:0.47), (TELMISARTAN:Ex-tobacco user:0.22), (THALIDOMIDE:Ex-tobacco user:0.43), (FOLIC ACID:Exostosis:0.08), (BUPRENORPHINE HYDROCHLORIDE:Extrapyramidal disorder:0.42), (BUPRENORPHINE HYDROCHLORIDENALOXONE HYDROCHLORIDE:Extrapyramidal disorder:0), (ESZOPICLONE:Extrapyramidal disorder:0.01), (LACOSAMIDE:Extrapyramidal disorder:0.08), (METHYLPHENIDATE HYDROCHLORIDE:Extrapyramidal disorder:0.2), (PRAMIPEXOLE DIHYDROCHLORIDE:Extrapyramidal disorder:0.25), (VARENICLINE TARTRATE:Extrapyramidal disorder:0.66), (ZONISAMIDE:Extrapyramidal disorder:0.02), (FERROUS SULFATEFERROUS SULFATE, DRIED:Extrasystoles:0.06), (ILOPROST:Extrasystoles:0.24), (PANTOPRAZOLE:Extrasystoles: 0.02), (SACUBITRILVALSARTAN:Extrasystoles:0.13), (UNSPECIFIED INGREDIENT:Extrasystoles:0.14), (ISOFLURANE:Extubation:0.16), (CARBOXYMETHYLCE LLULOSE SODIUMGLYCERINPOLYSORBATE 80:Eye allergy:0.56), (IBUPROFEN:Eye allergy:0.34), (LATANOPROSTTIMOLOL:Eye allergy:0.12), (ACARBOSE:Eye disorder:0.43), (ACETAMINOPHENOXYCODONEOXYCODONE HYDROCHLORIDE:Eye disorder:0.75), (ACETAMINOPHENPROPOXYPHENE HYDROCHLORIDE:Eye disorder:0.24), (ALBIGLUTIDE:Eye disorder:0.2), (ALENDRONIC ACID:Eye disorder:0.38), (ALFACALCIDOL:Eye disorder:0.3), (AMANTADINE HYDROCHLORIDE:Eye disorder:0.07), (AMISULPRIDE:Eye disorder:0.01), (AMLODIPINE BESYLATEATORVASTATIN CALCIUM:Eye disorder:0.01), (AMLODIPINEVALSARTAN:Eye disorder:0.39), (AMMONIUM LACTATE:Eye disorder:0.24), (BETAMETHASONE DIPROPIONATECALCIPOTRIENECALCIPOTRIENE HYDRATE:Eye disorder:0.09), (BETAMETHASONECALCIPOTRIENE:Eye disorder:0.02), (BUDESONIDEFORMOTEROL FUMARATE:Eye disorder:0.33), (BUPRENORPHINE HYDROCHLORIDENALOXONE HYDROCHLORIDE:Eye disorder:0.34), (BUTYLSCOPOLAMINE BROMIDE:Eye disorder:0.05), (CALCITONIN:Eye disorder:0.08), (CANAGLIFLOZINMETFORMIN HYDROCHLORIDE:Eye disorder:0.51), (CANAKINUMAB:Eye disorder:0.11), (CANDESARTAN:Eye disorder:1.44), (CANDESARTAN CILEXETILHYDROCHLOROTHIAZIDE:Eye disorder:1), (CARBOXYMETHYLCELLULOSE SODIUMHYPROMELLOSES:Eye disorder:0.45), (CARVEDILOL PHOSPHATE:Eye disorder:1.48), (CEFTRIAXONE SODIUM:Eye disorder:0.18), (CHLORHEXIDINE:Eye disorder:0.05), (CICLESONIDE:Eye disorder:0.15), (CILOSTAZOL:Eye disorder:0.39), (CINACALCET HYDROCHLORIDE:Eye disorder:0.45), (CLARITHROMYCIN:Eye disorder:0.26), (CLOTRIMAZOLE:Eye disorder:0.93), (CORTICOTROPIN:Eye disorder:0.53), (DACLIZUMAB:Eye disorder:0.64), (DEXAMETHASONENEOMYCIN SULFATEPOLYMYXIN B SULFATE:Eye disorder:0.03), (DEXTROAMPHETAMINE SULFATE:Eye disorder:0.11), (DICLOFENAC SODIUMMISOPROSTOL:Eye disorder:0.08), (DIPYRONE:Eye disorder:0.08), (DOXEPIN HYDROCHLORIDE:Eye disorder:0.69), (DRONABINOL:Eye disorder:0.66), (DRONEDARONE:Eye disorder:0.22), (EDOXABAN TOSYLATE:Eye disorder:0.28), (ELETRIPTAN HYDROBROMIDE:Eye disorder:1.37), (EMPAGLIFLOZINLINAGLIPTIN:Eye disorder:0.43), (EPLERENONE:Eye disorder:0.5), (ESOMEPRAZOLE MAGNESIUMNAPROXEN:Eye disorder:0.33), (FENTANYL CITRATE:Eye disorder:0.16), (FERROUS GLUCONATE:Eye disorder:0.16), (FEXOFENADINEPSEUDOEPHEDRINE:Eye disorder:0.23), (FLUDROCORTISONE ACETATE:Eye disorder:0.31), (FLUOCINOLONE ACETONIDE:Eye disorder:0.62), (FLUTICASONE FUROATESALMETEROL XINAFOATE:Eye disorder:0.2), (FLUVOXAMINE MALEATE:Eye disorder:0.04), (FORMOTEROL FUMARATE:Eye disorder:0.3), (FUSIDIC ACID:Eye disorder:0.28), (HYDROCHLOROTHIAZIDETELMISARTAN:Eye disorder:0.45), (HYDROCODONE BITARTRATEIBUPROFEN:Eye disorder:0.31), (HYDROXYZINE PAMOATE:Eye disorder:1.42), (ILOPROST:Eye disorder:0.73), (IMATINIB MESYLATE:Eye disorder:0.43), (INSULIN GLULISINE:Eye disorder:0.32), (IODINE:Eye disorder:0.1), (KETOROLACKETOROLAC TROMETHAMINE:Eye disorder:0.6), (LERCANIDIPINE:Eye disorder:0.93), (LEVALBUTEROL HYDROCHLORIDE:Eye disorder:1.43), (LINAGLIPTIN:Eye disorder:1.12), (LISDEXAMFETAMINE DIMESYLATE:Eye disorder:0.74), (LOMITAPIDE MESYLATE:Eye disorder:0.61), (LOPERAMIDE:Eye disorder:0.35), (METAXALONE:Eye disorder:2.04), (METHIMAZOLE:Eye disorder:0.25), (METHYLPREDNISOLONE ACETATE:Eye disorder:1.22), (METRONIDAZOLEMETRONIDAZOLE HYDROCHLORIDE:Eye disorder:0.81), (MOXIFLOXACIN:Eye disorder:0.74), (NALOXONE HYDROCHLORIDEOXYCODONE HYDROCHLORIDE:Eye disorder:0.92), (NAPHAZOLINE HYDROCHLORIDE:Eye disorder:0.36), (OLODATEROL HYDROCHLORIDETIOTROPIUM BROMIDE MONOHYDRATE:Eye disorder:0.42), (PARATHYROID HORMONE:Eye disorder:0.19), (PEGINTERFERON BETA-1A:Eye disorder:0.44), (PEMETREXED DISODIUM:Eye disorder:0.02), (PHENOBARBITAL:Eye disorder:1.09), (PILOCARPINE HYDROCHLORIDE:Eye disorder:0.14), (POLYETHYLENE GLYCOL 400:Eye disorder:0.06), (POLYVINYL ALCOHOLPOVIDONE:Eye disorder:0.56), (POVIDONE:Eye disorder:0.19), (PRAZOSIN:Eye disorder:0.05), (PREDNISOLONE ACETATESULFACETAMIDE SODIUM:Eye disorder:0.38), (PROTRIPTYLINE HYDROCHLORIDE:Eye disorder:0.02), (QUINAPRIL:Eye disorder:0.34), (RIVASTIGMINE TARTRATE:Eye disorder:0.37), (SCOPOLAMINE:Eye disorder:0.61), (SECUKINUMAB:Eye disorder:1.21), (SODIUM PHOSPHATE, DIBASICSODIUM PHOSPHATE, MONOBASIC:Eye disorder:0.14), (TERAZOSIN HYDROCHLORIDE:Eye disorder:0.18), (TERBUTALINE SULFATE:Eye disorder:0.13), (TETRACYCLINE:Eye disorder:1.29), (TIMOLOL MALEATETRAVOPROST:Eye disorder:0.47), (TROSPIUM CHLORIDE:Eye disorder:0), (UMECLIDINIUM BROMIDEVILANTEROL TRIFENATATE:Eye disorder:1.), (VANCOMYCIN HYDROCHLORIDE:Eye disorder:0.04), (VILAZODONE HYDROCHLORIDE:Eye disorder:0.55), (BUPROPION HYDROCHLORIDE:Eye infection:0.25), (CELECOXIB:Eye infection:0.39), (DICLOFENAC SODIUM:Eye infection:0.06), (DILTIAZEM:Eye infection:0.3), (DIPHENHYDRAMINE:Eye infection:0.32), (EPINEPHRINE:Eye infection:0.32), (FERROUS SULFATE:Eye infection:0.11), (LIDOCAINE:Eye infection:0), (NAPROXEN SODIUM:Eye infection:0.08), (NEBIVOLOL:Eye infection:0.45), (OXYGEN:Eye infection:0.15), (PANTOPRAZOLE:Eye infection:0.39), (SACUBITRILVALSARTAN:Eye infection:0.03), (TOPIRAMATE:Eye infection:0.12), (TRIAMCINOLONE ACETONIDE:Eye infection:0.31), (BUPROPION HYDROCHLORIDE:Eye inflammation:0.05), (EVOLOCUMAB:Eye inflammation:0.2), (TOPIRAMATE:Eye inflammation:0.13), (VALSARTAN:Eye inflammation:0.04), (MOXIFLOXACINTRIAMCINOLONE:Eye irrigation:0.04), (RIBOFLAVIN 5′-PHOSPHATE SODIUM:Eye irrigation:0.23), (TETRACAINETETRACAINE HYDROCHLORIDE:Eye irrigation:0.62), (CELECOXIB:Eye irritation:0), (DEXLANSOPRAZOLE:Eye irritation:0), (DEXTRAN 70\GLYCERINHYPROMELLOSES:Eye irritation:0.11), (DILTIAZEM:Eye irritation:0.15), (LATANOPROSTTIMOLOL:Eye irritation:0.49), (RAMIPRIL:Eye irritation:0.05), (SPIRONOLACTONE:Eye irritation:0.22), (TRIAMCINOLONE ACETONIDE:Eye irritation:0.12), (CARBOXYMETHYLCELLULOSE SODIUMGLYCERINPOLYSORBATE 80:Eye lubrication therapy:0.56), (ALBUTEROL:Eye pain:0.04), (CERTOLIZUMAB PEGOL:Eye pain:0.23), (DEXLANSOPRAZOLE:Eye pain:0.06), (DILTIAZEM:Eye pain:0.11), (DIVALPROEX SODIUM:Eye pain:0.42), (EVOLOCUMAB:Eye pain:0.06), (PANTOPRAZOLE:Eye pain:0.2), (SACUBITRILVALSARTAN:Eye pain:0.03), (SPIRONOLACTONE:Eye pain:0.06), (TAMSULOSIN:Eye pain:0.2), (BISACODYL OR DOCUSATE SODIUM:Eye pruritus:0), (BRINZOLAMIDE:Eye pruritus:0.02), (CERTOLIZUMAB PEGOL:Eye pruritus:0.2), (CLINDAMYCINCLINDAMYCIN PHOSPHATE:Eye pruritus:0.25), (CYCLOBENZAPRINE:Eye pruritus:0.3), (DASATINIB:Eye pruritus:0.09), (DIGOXIN:Eye pruritus:0.58), (ESZOPICLONE:Eye pruritus:0.15), (EVOLOCUMAB:Eye pruritus:0.67), (FERROUS SULFATEFERROUS SULFATE, DRIED:Eye pruritus:0.3), (IBRUTINIB:Eye pruritus:0.34), (INSULIN HUMAN:Eye pruritus:0.12), (IRBESARTAN:Eye pruritus:0.06), (LIDOCAINE:Eye pruritus:0.24), (METAXALONE:Eye pruritus:0.31), (PANTOPRAZOLE MAGNESIUM:Eye pruritus:0.19), (PHENOBARBITAL:Eye pruritus:0.45), (POLYETHYLENE GLYCOLS:Eye pruritus:0.22), (PRAMIPEXOLE DIHYDROCHLORIDE:Eye pruritus:0.09), (RABEPRAZOLE SODIUM:Eye pruritus:0.12), (RALOXIFENE HYDROCHLORIDE:Eye pruritus:0.34), (TETRACYCLINE:Eye pruritus:0.24), (UMECLIDINIUM BROMIDEVILANTEROL TRIFENATATE:Eye pruritus:0.21), (ZONISAMIDE:Eye pruritus:0.56),(ARMODAFINIL:Fabry's disease:0.51), (CALCIUM CARBONATECHOLECALCIFEROL:Fabry's disease:0.3), (CARVEDILOL PHOSPHATE:Fabry's disease:0.22), (CEFTRIAXONE SODIUM:Fabry's disease:0.01), (CERTOLIZUMAB PEGOL:Fabry's disease:0.44), (CLOTRIMAZOLE:Fabry's disease:0.17), (EVEROLIMUS:Fabry's disease:0.24), (GLIMEPIRIDE:Fabry's disease:0.05), (IBRUTINIB:Fabry's disease:0.55), (INSULIN ASPART:Fabry's disease:0.25), (INSULIN HUMAN:Fabry's disease:0.09), (LACOSAMIDE:Fabry's disease:0.33), (LEDIPASVIRSOFOSBUVIR:Fabry's disease:0.27), (LEVALBUTEROL HYDROCHLORIDE:Fabry's disease:0.42), (METHYLPREDNISOLONE ACETATE:Fabry's disease:0.29), (METRONIDAZOLE:Fabry's disease:0.76), (METRONIDAZOLEMETRONIDAZOLE HYDROCHLORIDE:Fabry's disease:0.04), (MOXIFLOXACIN HYDROCHLORIDE:Fabry's disease:0.08), (NILOTINIB:Fabry's disease:0.03), (OXYGEN:Fabry's disease:0.28), (PANTOPRAZOLE MAGNESIUM:Fabry's disease:0.12), (PAROXETINEPAROXETINE HYDROCHLORIDE:Fabry's disease:0.08), (PHENOBARBITAL:Fabry's disease:0.6), (PRIMIDONE:Fabry's disease:0.72), (PROBIOTICS NOS:Fabry's disease:0.22), (QUETIAPINE:Fabry's disease:0.03), (QUETIAPINE FUMARATE:Fabry's disease:0.07), (SODIUM OXYBATE:Fabry's disease:0.61), (SUMATRIPTAN:Fabry's disease:0.09), (SUMATRIPTANSUMATRIPTAN SUCCINATE:Fabry's disease:0.19), (TIZANIDINE HYDROCHLORIDE:Fabry's disease:0.45), (TOCILIZUMAB:Fabry's disease:0.03), (UMECLIDINIUM BROMIDEVILANTEROL TRIFENATATE:Fabry's disease:0.11), (VARENICLINE TARTRATE:Fabry's disease:0.37), (BUSPIRONE HYDROCHLORIDE:Facial pain:0.02), (DIVALPROEX SODIUM:Facial pain:0.46), (METRONIDAZOLE:Facial pain:0.57), (NAPROXEN:Facial pain:0.53), (OLANZAPINE:Facial pain:0.13), (VARENICLINE TARTRATE:Facial pain:0.13), (ANTIHEMOPHILIC FACTOR, HUMAN RECOMBINANT RESIDUES 743-1636 DELETED:Factor IX deficiency:0.34), (DILTIAZEM HYDROCHLORIDE:Factor V Leiden mutation:0.26), (ENALAPRIL:Factor V Leiden mutation:0.2), (EVOLOCUMAB:Factor V Leiden mutation:0.23), (HYDROXYCHLOROQUINE SULFATE:Factor V Leiden mutation:0.51), (LANSOPRAZOLE:Factor V Leiden mutation:0.1), (LOVASTATIN:Factor V Leiden mutation:0.1), (MELOXICAM:Factor V Leiden mutation:0.03), (NAPROXEN:Factor V Leiden mutation:0.62), (NEBIVOLOL:Factor V Leiden mutation:0.19), (OXYGEN:Factor V Leiden mutation:0.09), (PANTOPRAZOLE:Factor V Leiden mutation:0.18), (TAMSULOSIN:Factor V Leiden mutation:0.11), (TERIFLUNOMIDE:Factor V Leiden mutation:0.01), (TOCILIZUMAB:Factor V Leiden mutation:0.08), (VARENICLINE TARTRATE:Factor V Leiden mutation:0.11), (VERAPAMIL HYDROCHLORIDE:Factor V Leiden mutation:0.33), (COAGULATION FACTOR IX RECOMBINANT HUMAN:Factor VIII deficiency:0.62), (NAPROXEN:Factor VIII deficiency:0.18), (NEVIRAPINE:Factor VIII deficiency:0.01), (.ALPHA.1-PROTEINASE INHIBITOR HUMAN:Faeces hard:0.01), (ACETAMINOPHENOXYCODONEOXYCODONE HYDROCHLORIDE:Faeces hard:0.01), (AGALSIDASE BETA:Faeces hard:0.03), (ALBIGLUTIDE:Faeces hard:0.21), (ALENDRONIC ACID:Faeces hard:0.13), (AMOXICILLINCLAVULANATE POTASSIUM:Faeces hard:0.26), (BECLOMETHASONE DIPROPIONATE:Faeces hard:0.22), (BEVACIZUMAB:Faeces hard:0.02), (BIMATOPROST:Faeces hard:0.15), (BISOPROLOL FUMARATE:Faeces hard:0.43), (CANAGLIFLOZIN:Faeces hard:0.14), (CANAGLIFLOZINMETFORMIN HYDROCHLORIDE:Faeces hard:0.07), (CANDESARTAN:Faeces hard:1), (CANDESARTAN CILEXETILHYDROCHLOROTHIAZIDE:Faeces hard:0.43), (CERTOLIZUMAB PEGOL:Faeces hard:0.9), (CHOLESTYRAMINE:Faeces hard:0.71), (CLONIDINE HYDROCHLORIDE:Faeces hard:0.03), (CLOZAPINE:Faeces hard:0.26), (CORTICOTROPIN:Faeces hard:0.02), (DACLIZUMAB:Faeces hard:0.28), (DAPAGLIFLOZIN PROPANEDIOL:Faeces hard:0.33), (DASATINIB:Faeces hard:0.83), (EDOXABAN TOSYLATE:Faeces hard:0.16), (EMPAGLIFLOZINLINAGLIPTIN:Faeces hard:0.03), (ENALAPRIL MALEATE:Faeces hard:0.62), (ERYTHROPOIETIN:Faeces hard:0.77), (ESOMEPRAZOLE MAGNESIUMNAPROXEN:Faeces hard:0.06), (ESTROGENS, CONJUGATEDMEDROXYPROGESTERONE ACETATE:Faeces hard:0.39), (ESZOPICLONE:Faeces hard:0.7), (EVOLOCUMAB:Faeces hard:1.01), (FISH OIL\OMEGA-3 FATTY ACIDS:Faeces hard:0.21), (FLECAINIDE:Faeces hard:0.52), (FLUOXETINEFLUOXETINE HYDROCHLORIDE:Faeces hard:0.42), (FLUTICASONE FUROATE:Faeces hard:0.13), (GLYCOPYRRONIUMINDACATEROL:Faeces hard:0.03), (ILOPROST:Faeces hard:0.4), (INDAPAMIDE:Faeces hard:0.17), (INDOMETHACIN:Faeces hard:0.24), (INFLUENZA VIRUS VACCINE:Faeces hard:0.31), (INSULIN HUMAN:Faeces hard:0.58), (IRINOTECAN:Faeces hard:0.07), (LEDIPASVIRSOFOSBUVIR:Faeces hard:0.03), (LITHIUM CARBONATE:Faeces hard:0.53), (METAXALONE:Faeces hard:1.1), (METHENAMINE HIPPURATE:Faeces hard:0.62), (MOXIFLOXACIN HYDROCHLORIDE:Faeces hard:0.74), (NABUMETONE:Faeces hard:0.11), (NADOLOL:Faeces hard:0.04), (NEBIVOLOL:Faeces hard:0.22), (NILOTINIB:Faeces hard:0.62), (NINTEDANIB:Faeces hard:0.11), (PAROXETINEPAROXETINE HYDROCHLORIDE:Faeces hard:0.09), (PEGINTERFERON BETA-1A:Faeces hard:0.41), (PENICILLIN:Faeces hard:0.36), (PHENOBARBITAL:Faeces hard:0.6), (PONATINIB:Faeces hard:0.46), (QUETIAPINE:Faeces hard:0.46), (RABEPRAZOLE SODIUM:Faeces hard:0.42), (RALOXIFENE HYDROCHLORIDE:Faeces hard:0.46), (RANOLAZINE:Faeces hard:1.67), (RIZATRIPTAN BENZOATE:Faeces hard:0.46), (SACUBITRILVALSARTAN:Faeces hard:1.19), (SOTALOL:Faeces hard:0.87), (SUMATRIPTANSUMATRIPTAN SUCCINATE:Faeces hard:0.7), (TELMISARTAN:Faeces hard:0.63), (TETRACYCLINE:Faeces hard:0.06), (THALIDOMIDE:Faeces hard:0.41), (TOCILIZUMAB:Faeces hard:0.38), (TRIAMCINOLONE ACETONIDE:Faeces hard:0.48), (VANCOMYCIN:Faeces hard:0.08), (VILAZODONE HYDROCHLORIDE:Faeces hard:0.22), (VITAMIN A:Faeces hard:0.05), (ALBUTEROL SULFATEIPRATROPIUM BROMIDE:Faeces soft:0.05), (AMPHETAMINE ASPARTATEAMPHETAMINE SULFATEDEXTROAMPHETAMINE SACCHARATEDEXTROAMPHETAMINE SULFATE:Faeces soft:0.7), (BENAZEPRIL HYDROCHLORIDE:Faeces soft:0.02), (CALCIUM CARBONATECHOLECALCIFEROL:Faeces soft:0.58), (CANDESARTAN CILEXETIL:Faeces soft:0.01), (CEPHALEXIN:Faeces soft:0.29), (CERTOLIZUMAB PEGOL:Faeces soft:0.29), (DASATINIB:Faeces soft:0.23), (DEXLANSOPRAZOLE:Faeces soft:1.05), (DICLOFENAC:Faeces soft:0.39), (DIVALPROEX SODIUM:Faeces soft:0.11), (ENOXAPARIN:Faeces soft:0.09), (ERYTHROPOIETIN:Faeces soft:0.07), (EVEROLIMUS:Faeces soft:0.3), (EVOLOCUMAB:Faeces soft:0.6), (FLUTICASONE:Faeces soft:0.11), (IBRUTINIB:Faeces soft:1.07), (ILOPROST:Faeces soft:0.18), (INSULIN NOS:Faeces soft:0.03), (LIDOCAINEPRILOCAINE:Faeces soft:0.1), (LISDEXAMFETAMINE DIMESYLATE:Faeces soft:0.07), (METHYLPREDNISOLONE SODIUM SUCCINATE:Faeces soft:0.18), (METRONIDAZOLE:Faeces soft:0.33), (NILOTINIB:Faeces soft:0.01), (OLANZAPINE:Faeces soft:0.15), (PHENOBARBITAL:Faeces soft:0.15), (PRAMIPEXOLE DIHYDROCHLORIDE:Faeces soft:0.06), (TERIFLUNOMIDE:Faeces soft:0.64), (TIZANIDINE HYDROCHLORIDE:Faeces soft:0.51), (TORSEMIDE:Faeces soft:0.44), (VERAPAMIL HYDROCHLORIDE:Faeces soft:0.61), (VITAMIN B:Faeces soft:0.16), (.ALPHA.1-PROTEINASE INHIBITOR HUMAN:Fatigue:0.83), (ACETAMINOPHENDIPHENHYDRAMINE HYDROCHLORIDE:Fatigue:0.08), (AGALSIDASE BETA:Fatigue:0.56), (ALBIGLUTIDE:Fatigue:0.43), (ALCOHOL:Fatigue:0.65), (AMINO ACIDSDEXTROSEELECTROLYTES NOS:Fatigue:0.05), (AMISULPRIDE:Fatigue:0.43), (AMLODIPINE BESYLATEHYDROCHLOROTHIAZIDEOLMESARTAN MEDOXOMIL:Fatigue:0.44), (AMLODIPINE BESYLATEHYDROCHLOROTHIAZIDEVALSARTAN:Fatigue:0.08), (AMMONIUM LACTATE:Fatigue:0.41), (AMPICILLIN SODIUMSULBACTAM SODIUM:Fatigue:0.1), (ANTIHISTAMINES NOS:Fatigue:0.26), (ARIPIPRAZOLE LAUROXIL:Fatigue:0.12), (ATROPINEDIPHENOXYLATE:Fatigue:0.13), (AZELASTINE:Fatigue: 0.6), (BACITRACIN:Fatigue:0.02), (BENZTROPINE:Fatigue:0.15), (BETAMETHASONE DIPROPIONATE:Fatigue:0.16), (BETAMETHASONE DIPROPIONATECALCIPOTRIENECALCIPOTRIENE HYDRATE:Fatigue:0.43), (BETAMETHASONECALCIPOTRIENE:Fatigue:0.34), (BUPRENO RPHINE HYDROCHLORIDE:Fatigue:0.77), (BUTYLSCOPOLAMINE BROMIDE:Fatigue:0.04), (CALCIPOTRIENE:Fatigue:0.36), (CALCITONIN:Fatigue:0.49), (CA LCIUM CITRATEVITAMIN D:Fatigue:0.13), (CANAGLIFLOZINMETFORMIN HYDROCHLORIDE:Fatigue:0.78), (CAPSAICIN:Fatigue:0.05), (CARBIDOPA:Fatigue:0.03), (CASANTHRANOLDOCUSATE SODIUM:Fatigue:0.06), (CEFDINIR:Fatigue:0.04), (CETIRIZINE HYDROCHLORIDEPSEUDOEPHEDRINE HYDROCHLORIDE:Fatigue:0.68), (CHLORPHENIRAMINEHYDROCODONE:Fatigue:0.2), (CILASTATIN SODIUMIMIPENEM:Fatigue:0.21), (CIPROFIBRATE:Fatigue:0.12), (CLOZAPINE:Fatigue:1.12), (CODEINE PHOSPHATE:Fatigue:0.16), (CODEINE PHOSPHATEGUAIFENESIN:Fatigue:0.37), (DABIGATRAN:Fatigue:0.44), (DALTEPARIN: Fatigue:0.58), (DALTEPARIN SODIUM:Fatigue:0.37), (DANTROLENE SODIUM:Fatigue:0.21), (DIGITOXIN:Fatigue:0.21), (DOLASETRON MESYLATE:Fatigue:0.17), (DORZOLAMIDE:Fatigue:0.26), (DROSPIRENONEETHINYL ESTRADIOLLEVOMEFOLATE CALCIUM:Fatigue:0.05), (EDOXABAN TOSYLATE:Fatigue:0.91), (ELOTUZUMAB:Fatigue:0.19), (EMPAGLIFLOZINLINAGLIPTI N:Fatigue:0.53), (EPIRUBICIN:Fatigue:0.21), (EPLERENONE:Fatigue:0.72), (EVOLOCUMAB: Fatigue:2.05), (FORMOTEROL:Fatigue:0.19), (FUSIDIC ACID:Fatigue:0.61), (GEMCITABINE HYDROCHLORIDE:Fatigue:0.2), (GIMERACILOTERACILTEGAFUR:Fatigue:0.24), (GLYC ERIN:Fatigue:0.01), (GLYCOPYRRONIUM:Fatigue:0.06), (GUANFACINE:Fatigue:0.72), (HY ALURONATE SODIUM:Fatigue:0.09), (HYDROCHLOROTHIAZIDETELMISARTAN:Fatigue:0.77), (HYDR OCORTISONE ACETATE:Fatigue:0.22), (ILOPROST:Fatigue:0.3), (INFLIXIMAB-DYYB:Fatigue:0.05), (INSULIN DEGLUDEC:Fatigue:0.2), (INTERFERON ALFA-2A:Fatigue:0.28), (INTERFERON ALFA-2B: Fatigue:0.49), (IRINOTECAN:Fatigue:0.66), (ISOPROPYL ALCOHOLLEUPROLIDE ACETATENORETHINDRONE ACETATE:Fatigue:0.12), (KETOROLAC TROMETHAMINE:Fatigue:0.03), (LABETALOL HYDROCHLORIDE:Fatigue:0.34), (LAPATINIB DITOSYLATE:Fatigue:0.29), (LEVOCETIRIZINE:Fatigue:0.03), (LEVOLEUCOVORIN CALCIUM:Fatigue:0.19), (LOMITAPIDE MESYLATE:Fatigue:0.87), (MEFENAMIC ACID:Fatigue:0.39), (MEPOLIZUMAB:Fatigue:0.48), (METFORMIN HYDROCHLORIDEVILDAGLIPTIN:Fatigue:0.42), (METHIMAZOLE:Fatigue:0.58), (METO LAZONE:Fatigue:0.37), (METRELEPTIN:Fatigue:0.1), (MINOCYCLINE HYDROCHLORIDE:Fatigue:0.61), (MORPHINE SULFATENALTREXONE HYDROCHLORIDE:Fatigue:0.33), (MOXIFLOXACIN:Fatigue:0.15), (NALOXONE HYDROCHLORIDETILIDINE HYDROCHLORIDE:Fatigue:0.03), (NICORANDIL:Fatigue:0.06), (NIRAPARIB:Fatigue:0.67), (NORETHINDRONE:Fatigue:1.05), (OCTREOTIDE ACETATE:Fatigue:0.24), (OSPEMIFENE:Fatigue:0.56), (OXYBUTYNINOXYBUTYNIN CHLORIDE:Fatigue:0.16), (PANCRELIPASE:Fatigue:0.12), (PANTOPRAZOLE MAGNESIUM:Fatigue:1.71), (PARICALCITOL:Fatigue:0.13), (PATIROMER:Fatigue:0.45), (P EMETREXED DISODIUM:Fatigue:0.82), (PENTOXIFYLLINE:Fatigue:0.01), (PERTUZUMAB:Fatigue:0.09), (PHYTONADIONE:Fatigue:0.26), (PIRFENIDONE:Fatigue:0.14), (PRAZOSIN:Fatigue:0.86), (P RAZOSIN HYDROCHLORIDE:Fatigue:0.69), (PROPAFENONE:Fatigue:0.06), (PROPAFENONE HYDROCHLORIDE:Fatigue:0.5), (PROTRIPTYLINE HYDROCHLORIDE:Fatigue:0.34), (QUINAPRIL:Fatigue:0.44), (QUINAPRILQUINAPRIL HYDROCHLORIDE:Fatigue:0.18), (RADIATION THERAPY:Fatigue:0.44), (RAMUCIRUMAB:Fatigue:0.06), (REGORAFENIB:Fatigue:1.09), (S ALMETEROL XINAFOATE:Fatigue:0.43), (SAXAGLIPTIN HYDROCHLORIDE:Fatigue:0.37), (SECUKINUMAB:Fatigue:1.32), (SILVER SULFADIAZINE:Fatigue:0.09), (SODIUM FLUORIDE:Fatigue:0.44), (SODIUM PHOSPHATE, DIBASICSODIUM PHOSPHATE, MONOBASIC:Fatigue:0.25), (SOFOSBUVIRVELPATASVIR:Fatigue:0.02), (SOTALOL:Fatigue: 1.6), (TAMOXIFEN CITRATE:Fatigue:0.13), (TASIMELTEON:Fatigue:0.08), (TETRACYCLINE:Fatigue:1.21), (TH ALIDOMIDE:Fatigue:1.18), (TIMOLOL:Fatigue:1.11), (TIMOLOL MALEATE:Fatigue:0.29), (TOCOPHEROL:Fatigue:0.52), (TRIPTORELIN PAMOATE:Fatigue:0.07), (UMECLIDINIUM BROMIDE:Fatigue:0.43), (VANCOMYCIN HYDROCHLORIDE:Fatigue:0.06), (VINCRISTINE SULFATE:Fatigue:0.27), (VINORELBINE TARTRATE:Fatigue:0.29), (ZONISAMIDE:Febrile convulsion:0.1), (.ALPHA.-TOCOPHEROL:Febrile neutropenia:0.02), (ACETAMINOPHENHYDROCODONE:Febrile neutropenia:0.12), (ACETAMINOPHENPROPOXYPHENE NAPSYLATE:Febrile neutropenia:0.1), (ACTIVATED CHARCOAL:Febrile neutropenia:0.07), (ALBUTEROLIPRATROPIUM:Febrile neutropenia:0.23), (ALCOHOL:Febrile neutropenia:0.13), (ALENDRONIC ACID:Febrile neutropenia:0.54), (AMISULPRIDE:Febrile neutropenia:0.31), (AMMONIUM GLYCYRRHIZATECYSTEINE HYDROCHLORIDEGLYCINE:Febrile neutropenia:0.61), (AMPHETAMINE ASPARTATEAMPHETAMINE SULFATEDEXTROAMPHETAMINE SACCHARATEDEXTROAMPHETAMINE SULFATE:Febrile neutropenia:0), (AMSACRINE:Febrile neutropenia:0.47), (ANASTROZOLE:Febrile neutropenia:1.38), (ANIDULAFUNGIN:Febrile neutropenia:0.8), (ARSENIC TRIOXIDE:Febrile neutropenia:0.29), (ASPARAGINASE ERWINIA CHRYSANTHEMI:Febrile neutropenia:0.24), (ASPIRIN LYSINE:Febrile neutropenia:0.22), (AZOSEMIDE:Febrile neutropenia:0.24), (BECLOMETHASONE:Febrile neutropenia:0.15), (BENAZEPRIL HYDROCHLORIDE:Febrile neutropenia:0.12), (BENAZEPRILBENAZEPRIL HYDROCHLORIDE:Febrile neutropenia:0.28), (BETAMETHASONE BUTYRATE PROPIONATE:Febrile neutropenia:0.03), (BETAMIPRONPANIPENEM:Febrile neutropenia:0.91), (BRINZOLAMIDE:Febrile neutropenia:0.14), (BUDESONIDEFORMOTEROL FUMARATE:Febrile neutropenia:0), (CALCITONIN:Febrile neutropenia:0.14), (CALCITONIN SALMON:Febrile neutropenia:0.74), (CALCIUM CITRATE:Febrile neutropenia:0.26), (CALCIUMVITAMIN D:Febrile neutropenia:0.69), (CANAKINUMAB:Febrile neutropenia:0.87), (CANDESARTAN CILEXETILHYDROCHLOROTHIAZIDE:Febrile neutropenia:0.75), (CARVEDILOL PHOSPHATE:Febrile neutropenia:0.28), (CEFOTAXIMECEFOTAXIME SODIUM:Febrile neutropenia:0.43), (CERTOLIZUMAB PEGOL:Febrile neutropenia:0.89), (CHLORTHALIDONE:Febrile neutropenia:0.02), (CLOZAPINE:Febrile neutropenia:0.72), (CORTISONEHYDROCORTISONE:Febrile neutropenia:0.43), (CRIZOTINIB:Febrile neutropenia:0.14), (CYCLOBENZAPRINE HYDROCHLORIDE:Febrile neutropenia:0.4), (CYCLOPHOSPHAMIDECYTARABINEDEXAMETHASONEDOXORUB ICINMETHOTREXATEVINCRISTINE:Febrile neutropenia:0.22), (DABRAFENIB:Febrile neutropenia:0.28), (DEXAMETHASONE ACETATE:Febrile neutropenia:0.94), (DICYCLOMINE HYDROCHLORIDE:Febrile neutropenia:0.21), (DIETARY SUPPLEMENT:Febrile neutropenia:0.05), (DIGITOXIN:Febrile neutropenia:0.11), (DIPYRIDAMOLE:Febrile neutropenia:0.25), (DIVALPROEX SODIUM:Febrile neutropenia:0.57), (DOBUTAMINE HYDROCHLORIDE:Febrile neutropenia:0.74), (DOXAZOSIN MESYLATE:Febrile neutropenia:0.54), (DOXAZOSINDOXAZOSIN MESYLATE:Febrile neutropenia:0.23), (EDOXABAN TOSYLATE:Febrile neutropenia:0.94), (ELETRIPTAN HYDROBROMIDE:Febrile neutropenia:0.01), (ELOTUZUMAB:Febrile neutropenia:0.74), (EPLERENONE:Febrile neutropenia:0.17), (ERIBULIN:Febrile neutropenia:0.02), (ERYTHROMYCIN:Febrile neutropenia:1.07), (ETORICOXIB:Febrile neutropenia:0.24), (ETRETINATE:Febrile neutropenia:0.37), (FERROUS CITRATE:Febrile neutropenia:0.21), (FERROUS FUMARATE:Febrile neutropenia:0.13), (FLUINDIONE:Febrile neutropenia:0.05), (FLUOXETINEFLUOXETINE HYDROCHLORIDE:Febrile neutropenia:0.2), (FORMOTEROL:Febrile neutropenia:0.78), (FORMOTEROL FUMARATE DIHYDRATEMOMETASONE FUROATE:Febrile neutropenia:0.25), (FOSFLUCONAZOLE:Febrile neutropenia:0.93), (GLYCOPYRRONIUMINDACATEROL:Febrile neutropenia:0.49), (HYDROCHLOROTHIAZIDELISINOPRIL:Febrile neutropenia:0.39), (HYDROCHLOROTHIAZIDETELMISARTAN:Febrile neutropenia:0), (HYDROXYZINE PAMOATE:Febrile neutropenia:0.78), (IBRITUMOMAB TIUXETAN:Febrile neutropenia:1.51), (ILOPROST:Febrile neutropenia:0.09), (IMMUNE GLOBULIN NOS:Febrile neutropenia:0.19), (INDAPAMIDEPERINDOPRIL:Febrile neutropenia:0.7), (INTERFERON GAMMA-1B:Febrile neutropenia:0.54), (ISOSORBIDE:Febrile neutropenia:0.31), (LACTOBACILLUS RHAMNOSUS:Febrile neutropenia:0.22), (LAPATINIB DITOSYLATE:Febrile neutropenia:0.06), (LEDIPASVIRSOFOSBUVIR:Febrile neutropenia:0.3), (LERCANIDIPINE HYDROCHLORIDE:Febrile neutropenia:0.33), (LEVALBUTEROL HYDROCHLORIDE:Febrile neutropenia:0.85), (LOMUSTINE:Febrile neutropenia:0.01), (METAXALONE:Febrile neutropenia:0.14), (METHENAMINE HIPPURATE:Febrile neutropenia:0), (METHIMAZOLE:Febrile neutropenia:0.32), (NEBIVOLOL:Febrile neutropenia:0.31), (NELARABINE:Febrile neutropenia:0.8), (NICARDIPINE:Febrile neutropenia:0.38), (NORTRIPTYLINE:Febrile neutropenia:0.17), (OLAPARIB:Febrile neutropenia:0.07), (OLODATEROL HYDROCHLORIDETIOTROPIUM BROMIDE MONOHYDRATE:Febrile neutropenia:0.36), (OXCARBAZEPINE:Febrile neutropenia:1.18), (PALIFERMIN:Febrile neutropenia:0.13), (PANITUMUMAB:Febrile neutropenia:0.25), (PANTOPRAZOLE MAGNESIUM:Febrile neutropenia:0.74), (PARICALCITOL:Febrile neutropenia:0.25), (PAROXETINE:Febrile neutropenia:0.02), (PAROXETINEPAROXETINE HYDROCHLORIDE:Febrile neutropenia:0.06), (PAZOPANIB HYDROCHLORIDE:Febrile neutropenia:0.59), (PAZUFLOXACIN:Febrile neutropenia:0.22), (PEMETREXED:Febrile neutropenia:0.8), (PENICILLIN:Febrile neutropenia:0.9), (PENICILLIN V:Febrile neutropenia:0.78), (PENTOSTATIN:Febrile neutropenia:0.98), (POTASSIUM CITRATETRISODIUM CITRATE DIHYDRATE:Febrile neutropenia:0.39), (PRAMIPEXOLE DIHYDROCHLORIDE:Febrile neutropenia:0.15), (PREDNISOLONE SODIUM PHOSPHATE:Febrile neutropenia:0.73), (PRIMIDONE:Febrile neutropenia:0.4), (RADIATION THERAPY:Febrile neutropenia:0.53), (RANOLAZINE:Febrile neutropenia:0.39), (REGORAFENIB:Febrile neutropenia:0.32), (ROFLUMILAST:Febrile neutropenia:0.53), (SECUKINUMAB:Febrile neutropenia:0.16), (SODIUM FERROUS CITRATE:Febrile neutropenia:0.38), (SOTALOL:Febrile neutropenia:0.86), (SUFENTANIL CITRATE:Febrile neutropenia:0.14), (SULFAMERAZINETRIMETHOPRIM:Febrile neutropenia:0.49), (TENIPOSIDE:Febrile neutropenia:0.13), (TERBUTALINE:Febrile neutropenia:0.45), (TETRACYCLINE:Febrile neutropenia:0.46), (TINZAPARIN:Febrile neutropenia:0.16), (TIZANIDINE:Febrile neutropenia:0.21), (TIZANIDINE HYDROCHLORIDE:Febrile neutropenia:0.26), (TOCILIZUMAB:Febrile neutropenia:0), (TRAMETINIB:Febrile neutropenia:0.28), (VINORELBINE TARTRATE:Febrile neutropenia:0.23), (VINORELBINEVINORELBINE TARTRATE:Febrile neutropenia:0.05), (VITAMIN A:Febrile neutropenia:0.04), (VITAMIN B:Febrile neutropenia:0.38), (PYRAZINAMIDE:Feeding disorder:0.57), (DIVALPROEX SODIUM:Feeling abnormal:0.57), (EVOLOCUMAB:Feeling abnormal:0.05), (TERIFLUNOMIDE:Feeling abnormal:0.31), (TOPIRAMATE:Feeling abnormal:0.54), (ALBUTEROL SULFATEIPRATROPIUM BROMIDE:Feeling of relaxation:0.09), (AMOXICILLIN:Feeling of relaxation:0.04), (ANASTROZOLE:Feeling of relaxation:0.21), (BISOPROLOL FUMARATE:Feeling of relaxation:0.14), (BUMETANIDE:Feeling of relaxation:0.23), (CALCIUM CARBONATECHOLECALCIFEROL:Feeling of relaxation:0.33), (CANDESARTAN:Feeling of relaxation:0.05), (CHOLESTYRAMINE:Feeling of relaxation:0.19), (COLCHICINE:Feeling of relaxation:0.16), (DALFAMPRIDINE:Feeling of relaxation:0.64), (DUTASTERIDE:Feeling of relaxation:0.14), (ELETRIPTAN HYDROBROMIDE:Feeling of relaxation:0.07), (ENOXAPARIN:Feeling of relaxation:0.26), (ESZOPICLONE:Feeling of relaxation:0.34), (EVEROLIMUS:Feeling of relaxation:0.14), (EVOLOCUMAB:Feeling of relaxation:0.4), (GLIMEPIRIDE:Feeling of relaxation:0.51), (HYDROCHLOROTHIAZIDELISINOPRIL:Feeling of relaxation:0.21), (HYDROMORPHONE HYDROCHLORIDE:Feeling of relaxation:0.17), (IBRUTINIB:Feeling of relaxation:0.42), (INSULIN ASPART:Feeling of relaxation:0.47), (INSULIN LISPRO:Feeling of relaxation:0.22), (LISDEXAMFETAMINE DIMESYLATE:Feeling of relaxation:0.7), (LITHIUM CARBONATE:Feeling of relaxation:0.41), (METAXALONE:Feeling of relaxation:0.33), (METHYLPREDNISOLONE SODIUM SUCCINATE:Feeling of relaxation:0.13), (METRONIDAZOLE:Feeling of relaxation:0.45), (MODAFINIL:Feeling of relaxation:0.19), (NAPROXEN:Feeling of relaxation:1.05), (NEBIVOLOL:Feeling of relaxation:0.01), (NITROFURANTOIN:Feeling of relaxation:0.03), (OMEGA-3-ACID ETHYL ESTERS:Feeling of relaxation:0.05), (PAROXETINEPAROXETINE HYDROCHLORIDE:Feeling of relaxation:0.31), (PRAVASTATIN SODIUM:Feeling of relaxation:0.3), (PRIMIDONE:Feeling of relaxation:0.33), (SACUBITRILVALSARTAN:Feeling of relaxation:0.22), (TERIFLUNOMIDE:Feeling of relaxation:0.29), (TOCILIZUMAB:Feeling of relaxation:0.02), (TRIAMCINOLONE ACETONIDE:Feeling of relaxation:0.08), (UMECLIDINIUM BROMIDEVILANTEROL TRIFENATATE:Feeling of relaxation:0.34), (VERAPAMIL HYDROCHLORIDE:Feeling of relaxation:0.53), (VILAZODONE HYDROCHLORIDE:Feeling of relaxation:0.04), (VITAMIN B:Feeling of relaxation:0.15), (ZONISAMIDE:Feeling of relaxation:0.13), (ABIRATERONE ACETATE:Fibromyalgia:0.15), (ABOBOTULINUMTOXINA:Fibromyalgia:0.51), (ACETAMI NOPHENCAFFEINECARISOPRODOLDICLOFENAC SODIUM:Fibromyalgia:0.93), (ADO-TRASTUZUMAB EMTANSINE:Fibromyalgia:0.1), (AGALSIDASE BETA:Fibromyalgia:1.44), (ALCOHOLALUMINUM CHLORIDE:Fibromyalgia:0.32), (ALEMTUZUMAB:Fibromyalgia:0.53), (ALENDRONIC ACID:Fibromyalgia:1.4), (ALFALFA:Fibromyalgia:0.32), (ALISKIREN HEMIFUMARATEHYDROCHLOROTHIAZIDE:Fibromyalgia:0.49), (ALLANTOINBENZO CAINECAMPHOR (NATURAL)DIMETHICONEMENTHOL:Fibromyalgia:0.12), (ALUMINUM HYDROXIDEMAGNESIUM HYDROXIDESIMETHICONE:Fibromyalgia:0.53), (ANAKINRA:Fibromyalgia:0.55), (APPLE CIDER VINEGAR:Fibromyalgia:0.36), (ARIPIPRAZOLE LAUROXIL:Fibromyalgia:0.72), (ASCORBIC ACIDBIOTINCALCIUM PANTOTHENATECOBALAMINFOLIC ACIDNIACINAMIDEPYRIDOXINE HYDROCHLORIDERIBOFLAVINTHIAMINE MONONITRATE:Fibromyalgia:0.27), (ASCORBIC ACIDBIOTINCYANOCOBALAMINFOLIC ACIDNIACINPANTOTHENIC ACIDPYRIDOXINERIBOFLAVINTHIAMINE:Fibromyalgia:0.06), (ASPIRINCAFFEINES ALICYLAMIDE:Fibromyalgia:0.73), (AXITINIB:Fibromyalgia:0.51), (AZILSARTAN KAMEDOXOMIL:Fibromyalgia:0.71), (AZITHROMYCIN MONOHYDRATE:Fibromyalgia:0.61), (AZTREONAM:Fibromyalgia:0.25), (BACITRACIN ZINC:Fibromyalgia:0.08), (BECLOMETHASONE:Fibromyalgia:0.94), (BENZOIC ACIDHYOSCYAMINE SULFATEMETHENAMINEMETHYLENE BLUEPHENYL SALICYLATE:Fibromyalgia:0.1), (BEPOTASTINE BESILATE:Fibromyalgia:0.07), (BETAMETHASONE DIPROPIONATECALCIPOTRIENE:Fibromyalgia:0.58), (BETAMETHASONE DIPROPIONATECALCIPOTRIENE HYDRATE:Fibromyalgia:0.06), (BETAMETHASONE DIPROPIONATECALCIPOTRIENECALCIPOTRIENE HYDRATE:Fibromyalgia:2.03), (BETAMETHASONECALCIPOTRIENE:Fibromyalgia:1.44), (BIFIDOBACTERIUM ANIMALIS LACTIS:Fibromyalgia:0.45), (BIPERIDEN HYDROCHLORIDE:Fibromyalgia:0.22), (BOCEPREVIR:Fibromyalgia:0.01), (BRINZOLAMI DETIMOLOL MALEATE:Fibromyalgia:0.26), (BUTORPHANOL:Fibromyalgia:0.52), (CABOZANTINIB S-MALATE:Fibromyalgia:0.05), (CAJUPUT OILEUCALYPTUS OILPEPPERMINT OIL:Fibromyalgia:0.27), (CALCIUM CARBONATEERGOCALCIFEROLRETINOL:Fibromyalgia:0.51), (CALCIUM CARBONATEETIDRONATE DISODIUM:Fibromyalgia:0.45), (CALCIUM CARBONATEMAGNESIUM HYDROXIDE:Fibromyalgia:0.21), (CANAKINUMAB:Fibromyalgia:1.24), (CAPSICUM:Fibromyalgia: 0.01), (CARBAMIDE PEROXIDE:Fibromyalgia:0.44), (CARBOXYMETHYLCELLULOSE SODIUMGLYCERINPOLYSORBATE 80:Fibromyalgia:0.41), (CEFAZOLIN:Fibromyalgia:0.55), (CEFAZOLIN SODIUM:Fibromyalgia:0.8), (CEFOTAXIME SODIUM:Fibromyalgia:0.31), (CEFPROZIL:Fibromyalgia:0.52), (CHAMOMILE:Fibromyalgia: 0.11), (CINACALCET HYDROCHLORIDE:Fibromyalgia:1.33), (CLIKSTAR:Fibromyalgia:0.78), (CODEINE PHOSPHATEPROMETHAZINE:Fibromyalgia:0.05), (DACLIZUMAB:Fibromyalgia:1.75), (D ALTEPARIN:Fibromyalgia:1.24), (DALTEPARIN SODIUM:Fibromyalgia:0.12), (DEXAMETHASONE ACETATE:Fibromyalgia:0.11), (DICHLORPHENAMIDE:Fibromyalgia:0.16), (DICLOXACILL IN:Fibromyalgia:0.13), (DIGITOXIN:Fibromyalgia:0.12), (DIMETHICONE 410:Fibromyalgia:0.5), (DIMETHYL SULFOXIDE:Fibromyalgia:0.02), (DIPHENHYDRAMINE HYDROCHLORIDEDOXYLAMINE SUCCINATE:Fibromyalgia:0.09), (DOLASETRON MESYLATE:Fibromyalgia:0.43), (DROSPIRENONEETHINYL ESTRADIOLLEVOMEFOLATE CALCIUM:Fibromyalgia:0.55), (ELOTUZUMAB:Fibromyalgia:0.2), (EPHEDRINE HYDROCHLORIDEGUAIFENESIN:Fibromyalgia:0.65), (ERTAPENEM SODIUM:Fibromyalgia:0.08), (ESTRADIOL BENZOATE:Fibromyalgia:1.09), (ESTRADIOL HEMIHYDRATE:Fibromyalgia:0.82), (ETHINYL ESTRADIOLFERROUS FUMARATENORETHINDRONE:Fibromyalgia:0.26), (EUCALYPTOLMENTHOLMETHY L SALICYLATETHYMOL:Fibromyalgia:0.35), (FENOTEROL HYDROBROMIDEIPRATROPIUM BROMIDE:Fibromyalgia:0.37), (FERRIC POLYSACCHARIDE COMPLEX:Fibromyalgia:0.62), (FERROUS SULFATE ANHYDROUS:Fibromyalgia:0.65), (FERROUS SULFATEGLYCINE:Fibromyalgia:0.44), (FLUCLOXACILLIN:Fibromyalgia:0.06), (FLUINDI ONE:Fibromyalgia:0.03), (FLUNARIZINE HYDROCHLORIDE:Fibromyalgia:0.58), (FLUPHENAZINE:Fibromyalgia:0.09), (FORMOTER OL FUMARATEMOMETASONE FUROATE:Fibromyalgia:1.69), (GARLIC OIL:Fibromyalgia:0.11), (GLIPIZIDEMETFORMIN HYDROCHLORIDE:Fibromyalgia:0.86), (GLYCOPYRRONIUMINDACATEROL:Fibromyalgia: 1.71), (GREEN TEA LEAF:Fibromyalgia:0.1), (GUAIFENESINPHENYLEPHRINE HYDROCHLORIDE:Fibromyalgia:0.31), (HALOBETASOL PROPIONATE:Fibromyalgia:0.48), (HALOPERIDOL DECANOATE:Fibromyalgia:0.21), (HERBALSHOMEOPATHICSGOLDENSEAL:Fibromyalgia: 0.02), (HERBALSSAW PALMETTO:Fibromyalgia:0.16), (HIGH DENSITY POLYETHYLENE:Fibromyalgia:0.15), (HORSE CHESTNUT:Fibromyalgia:0.98), (HYDROCHLORIC ACID:Fibromyalgia:0.08), (HYDROCORTISONE ACETATEPRAMOXINE HYDROCHLORIDE:Fibromyalgia:0.12), (HYDROGEN PEROXIDE:Fibromyalgia:0.06), (IMIPRAMINE PAMOATE:Fibromyalgia:0.32), (INDACATEROL MALEATE:Fibromyalgia:0.08), (INDAPAMIDEPERINDOPRIL:Fibromyalgia:1.27), (INFLIXI MAB-DYYB:Fibromyalgia:0.75), (INSULIN BEEF:Fibromyalgia:1.1), (INSULIN PORKINSULIN PURIFIED PORK:Fibromyalgia:0.21), (INTERFERON:Fibromyalgia:0.59), (INTERFERON ALFA-2A:Fibromyalgia:0.27), (INTERFERON GAMMA-1B:Fibromyalgia:0.91), (ITRACONAZOLESODIUM CHLORIDE:Fibromyalgia:0.09), (IVABRADINE:Fibromyalgia:0.08), (IVABRADINE HYDROCHLORIDE:Fibromyalgia:0.24), (IVACAFTOR:Fibromyalgia:0.44), (IXAZOMIB: Fibromyalgia:0.81), (LAPATINIB DITOSYLATE:Fibromyalgia:0.06), (LATANOPROSTTIMOLOL:Fibromyalgia:0.06), (LERCA NIDIPINE:Fibromyalgia:1.49), (LEVOMEFOLIC ACIDMETHYLCOBALAMINTURMERIC:Fibromyalgia:0.14), (LEVOTHYROXINE SODIUMPOTASSIUM IODIDE:Fibromyalgia:0.13), (LOMITAPIDE MESYLATE:Fibromyalgia:1.76), (LUTETIUM LU-177:Fibromyalgia:0.28), (MECLOZINE DIHYDROCHLORIDE:Fibromyalgia:0.34), (MEGESTROL:Fibromyalgia:0.39), (MENAQUIN ONE 6:Fibromyalgia:0.54), (METHENAMINE MANDELATE:Fibromyalgia:1.02), (METRELEPTIN:Fibromyalgia:0.32), (MOEXIPRIL HYDROCHLORIDE:Fibromyalgia:1.15), (MOXIFLOXACIN:Fibromyalgia:1.29), (NADROPA RIN CALCIUM:Fibromyalgia:0.18), (NAFTIFINE HYDROCHLORIDE:Fibromyalgia:0.29), (NICARDIPINE:Fibromyalgia:0.24), (NICORANDIL: Fibromyalgia:0.01), (NIRAPARIB:Fibromyalgia:0.22), (NOREPINEPHRINE BITARTRATE:Fibromyalgia:0.01), (OMEGA-6 FATTY ACIDS:Fibromyalgia:0.18), (ORLISTAT:Fibromyalgia:0.22), (OSELTAMIVIR:Fibromyalgia:0.51), (PAPAYA:Fibromyalgia:0.6), (PARSLEY:Fibromyalgia:0), (PAZOPANIB HYDROCHLORIDE:Fibromyalgia:1.31), (PEGINTERFERON BETA-1A:Fibromyalgia:1.92), (PEMETREXED DISODIUM:Fibromyalgia:0.06), (PENICILLIN G:Fibromyalgia:0.96), (PHENYLEPHRINE HYDROCHLORIDEPROMETHAZINE HYDROCHLORIDE:Fibromyalgia:0.27), (PHENYLEPHRINE HYDROCHLORIDEWITCH HAZEL:Fibromyalgia:0.02), (PODOFILOX:Fibromyalgia:0.28), (POLYMYXIN B SULFATETRIMETHOPRIM SULFATE:Fibromyalgia:0.51), (POLYVINYL ALCOHOL:Fibromyalgia:0.07), (PONATINIB:Fibromyalgia:1.38), (POVIDONE:Fibromyalgia:00.35), (PREDNISOLONE ACETATESULFACETAMIDE SODIUM:Fibromyalgia:0.08), (PREDNISOLONE SODIUM PHOSPHATE:Fibromyalgia:0.74), (PROPATYL NITRATE:Fibromyalgia:0.45), (PRUCALOPRIDE SUCCINATE:Fibromyalgia:0.18), (PYRAZINAMIDE:Fibromyalgia:0.76), (QUERCETIN:Fibromyalgia: 0.03), (QUINACRINE:Fibromyalgia:1.18), (QUINIDINE GLUCONATE:Fibromyalgia:0.62), (SERRAPEPTASE:Fibromyalgia:0.3), (SIBUTRAMINE HYDROCHLORIDE:Fibromyalgia:1.24), (SODIUM BICARBONATESODIUM CHLORIDE:Fibromyalgia:0.48), (SODIUM FLUORIDEVITAMINS:Fibromyalgia:0.13), (TAMOXIFEN CITRATE:Fibromyalgia:0.15), (TASIMELTEON:Fibromyalgia:1.04), (TAZAROTENE:Fibromyalgia: 0.32), (TEDUGLUTIDE:Fibromyalgia:0.37), (TEDUGLUTIDEWATER:Fibromyalgia:1.16), (TERBUTALINE SULFATE:Fibromyalgia:2.42), (THALIDOMIDE:Fibromyalgia:1.44), (TIBOLONE:Fibromyalgia: 1.27), (TILIDINE:Fibromyalgia:0.31), (TIOPRONIN:Fibromyalgia:0.05), (TOLMETIN SODIUM:Fibromyalgia:0.09), (TRIPTORELIN PAMOATE:Fibromyalgia:0.11), (VALDECOXIB:Fibromyalgia:0.91), (VORICONAZOLE:Fibromyalgia: 0.14), (ZEAXANTHIN:Fibromyalgia:0.08), (METRONIDAZOLE:Flank pain:0), (.ALPHA.1-PROTEINASE INHIBITOR HUMAN:Flatulence:0.59), (ACETAMINOPHENOXYCODONE HYDROCHLORIDEOXYCODONE TEREPHTHALATE:Flatulence:0.05), (ACETAMINOPHENOXYCODONEOXYCODONE HYDROCHLORIDE:Flatulence:0.55), (ACETAMINOPHENPROPOXYPHENE HYDROCHLORIDE:Flatulence:0), (ADO-TRASTUZUMAB EMTANSINE:Flatulence:0.25), (AGALSIDASE BETA:Flatulence:0.05), (ALCOHOL:Flatulence:0.11), (ALENDRONIC ACID:Flatulence:0.36), (ALFACALCIDOL:Flatulence:0.07), (ALISKIREN HEMIFUMARATE:Flatulence:0.04), (AMINO ACIDSDEXTROSEELECTROLYTES NOS:Flatulence:0.56), (AMITRIPTYLINE HYDROCHLORIDE:Flatulence:0.78), (AMLODIPINE BESYLATEHYDROCHLOROTHIAZIDEOLMESARTAN MEDOXOMIL:Flatulence:0.26), (AMLODIPINE BESYLATEOLMESARTAN MEDOXOMIL:Flatulence:0.05), (AMLODIPINEVALSARTAN:Flatulence:0.09), (BALSALAZ IDE DISODIUM:Flatulence:0.01), (BENAZEPRILBENAZEPRIL HYDROCHLORIDE:Flatulence:0.48), (BICALUTAMIDE:Flatulence:0.35), (CALCITONIN:Flatulence: 0.25), (CALCITONIN SALMON:Flatulence:0.61), (CANAGLIFLOZINMETFORMIN HYDROCHLORIDE:Flatulence:0.36), (CANDESARTAN CILEXETIL:Flatulence:1.06), (CARFILZOMIB:Flatulence:0.83), (CARVEDILOL PHOSPHATE:Flatulence:0.71), (CEFOTAXIME SODIUM:Flatulence:0.01), (CILASTATIN SODIUMIMIPENEM:Flatulence:0.06), (CLARITHROMYCIN:Flatulence:0.05), (CLINDAMY CIN PHOSPHATE:Flatulence:0.05), (CLINDAMYCINCLINDAMYCIN PHOSPHATE:Flatulence:1.66), (CORTICOTROPIN:Flatulence:0.36), (CYPROHEPTADINE: Flatulence:0.37), (DACLIZUMAB:Flatulence:0.56), (DANAZOL:Flatulence:0.34), (DEFLAZACO RT:Flatulence:0.3), (DESLORATADINE:Flatulence:0.51), (DOCETAXEL:Flatulence:0.46), (DO XAZOSIN MESYLATE:Flatulence:0.74), (DULAGLUTIDE:Flatulence:0.35), (DUTASTERIDE:Flatulence: 0.55), (EMPAGLIFLOZINLINAGLIPTIN:Flatulence:0.43), (EPLERENONE:Flatulence:0.13), (ERYTHROMYCIN:Flatulence:0.74), (ESOMEPRAZOLE MAGNESIUMNAPROXEN:Flatulence:0.28), (ESTROGENS, CONJUGATEDMEDROXYPROGESTERONE ACETATE:Flatulence:0.58), (EVOLOCUMAB:Flatulence:1.12), (FESOTERODINE FUMARATE:Flatulence:0.63), (FLUDROCORTISONE ACETATE:Flatulence:0.39), (FLUTICASONE FUROATE:Flatulence:0.66), (FLUTICASONE FUROATEVILANTEROL TRIFENATATE:Flatulence:0.66), (FORMOTEROL FUMARATEMOMETASONE FUROATE:Flatulence:0.49), (FUSIDIC ACID:Flatulence:0.35), (GLYCOPYRRONIUM:Flatulence:0.02), (GLYCOPYRRONIUMINDA CATEROL:Flatulence:0.3), (HYDROCHLOROTHIAZIDETELMISARTAN:Flatulence:0.25), (HYDROCORTISONE ACETATE:Flatulence:0.18), (ILOPROST:Flatulence:0.27), (IMATINIB MESYLATE:Flatulence:1.21), (IMIPRAMINE HYDROCHLORIDE:Flatulence:0.51), (INDAPAMIDEPERINDOPRIL:Flatulence:0.51), (INSU LIN GLULISINE:Flatulence:0.18), (IRINOTECAN HYDROCHLORIDE:Flatulence:0.41), (IXAZOMIB:Flatulence:0.11), (LACOSAMIDE:Flatulence: 0.42), (LACTASE:Flatulence:0.26), (LEDIPASVIRSOFOSBUVIR:Flatulence:0.59), (LEVOLE UCOVORIN CALCIUM:Flatulence:0.18), (LIOTHYRONINE:Flatulence:0.04), (LISDEXAMFETAMINE DIMESYLATE:Flatulence:0.52), (LOMITAPIDE MESYLATE:Flatulence:0.38), (MEPOLIZUMAB:Flatulence:0.2), (METHENAMINE HIPPURATE:Flatulence:0.95), (METHIMAZOLE:Flatulence:0.43), (METHYLPREDNISOLON E ACETATE:Flatulence:1.09), (MOXIFLOXACIN:Flatulence:0.12), (NATEGLINIDE:Flatulence: 0.13), (NILOTINIB:Flatulence:1.31), (NORETHINDRONE:Flatulence:0.2), (PANTOPRAZOLE MAGNESIUM:Flatulence:1.76), (PATIROMER:Flatulence:0.11), (PEGINTERFERON BETA-1A:Flatulence:0.67), (PHENYTOIN:Flatulence:0.52), (PREDNISOLONE ACETATE:Flatulence:0.63), (PREDNISOLONEPREDNISOLONE ACETATEPREDNISOLONE SODIUM SUCCINATE:Flatulence:0.05), (RISEDRONATE SODIUM:Flatulence:1.06), (SACUBITRILVALSARTAN:Flatulence:0.58), (SCOPOLAMINE: Flatulence:0.62), (SITAGLIPTIN:Flatulence:0.1), (SOTALOL:Flatulence:1.28), (SUMATRIPTAN SUCCINATE:Flatulence:0.06), (TERBINAFINE HYDROCHLORIDE:Flatulence:0.28), (TERBUTALINE SULFATE:Flatulence:0.63), (TERIFLUNOMIDE:Flatulence:0.97), (TETRACYCLINE:Flatulence: 0.79), (TINZAPARIN SODIUM:Flatulence:0.12), (TIOTROPIUM:Flatulence:0.29), (TOCOPHEROL:Flatulence:0.19), (UMECLIDINIUM BROMIDE:Flatulence:0.03), (UMECLIDINIUM BROMIDEVILANTEROL TRIFENATATE:Flatulence:0.68), (VEMURAFENIB:Flatulence:0.03), (ZAFIRLUKAST:Flatulence: 0.16), (AMIODARONE HYDROCHLORIDE:Fluid imbalance:0.29), (AMOXICILLIN:Fluid imbalance:0.05), (AMPHETAMINE ASPARTATEAMPHETAMINE SULFATEDEXTROAMPHETAMINE SACCHARATEDEXTROAMPHETAMINE SULFATE:Fluid imbalance:0.31), (CANDESARTAN CILEXETIL:Fluid imbalance:0.19), (CERTOLIZUMAB PEGOL:Fluid imbalance:0.01), (CINACALCET HYDROCHLORIDE:Fluid imbalance:0.13), (COLCHICINE:Fluid imbalance:0.19), (ESCITALOPRAM OXALATE:Fluid imbalance:0.63), (EVOLOCUMAB:Fluid imbalance:1.03), (ILOPROST:Fluid imbalance:0.44), (IMATINIB MESYLATE:Fluid imbalance:0.12), (IRON:Fluid imbalance:0.6), (ISOSORBIDE:Fluid imbalance:0.2), (LORATADINE:Fluid imbalance:0.25), (MAGNESIUM:Fluid imbalance:0.52), (METAXALONE:Fluid imbalance:0.45), (METRONIDAZOLE:Fluid imbalance:0.19), (OXCARBAZEPINE:Fluid imbalance:0.03), (PANTOPRAZOLE MAGNESIUM:Fluid imbalance:0.05), (RALOXIFENE HYDROCHLORIDE:Fluid imbalance:0.05), (RAMIPRIL:Fluid imbalance:1.04), (SACUBITRILVALSARTAN:Fluid imbalance:0.29), (SELEXIPAG:Fluid imbalance:0.14), (TOPIRAMATE:Fluid imbalance:0.51), (ANASTROZOLE:Fluid overload:0.46), (ATOVAQUONE:Fluid overload:0.28), (BISACODYL OR DOCUSATE SODIUM:Fluid overload:0.43), (CALCIUM CARBONATECHOLECALCIFEROL:Fluid overload:0.96), (CANDESARTAN CILEXETILHYDROCHLOROTHIAZIDE:Fluid overload:0.43), (CEFOTAXIME SODIUM:Fluid overload:0.23), (CERTOLIZUMAB PEGOL:Fluid overload:0.75), (CHOLESTYRAMINE:Fluid overload:0.5), (CLINDAMYCIN:Fluid overload:0.05), (CORTISONEHYDROCORTISONE:Fluid overload:0.16), (DASATINIB:Fluid overload:0.91), (DEVICE:Fluid overload:0.29), (DICLOFENAC:Fluid overload:0.31), (DICLOFENAC SODIUM:Fluid overload:0.11), (DOXAZOSIN MESYLATE:Fluid overload:0.07), (DUTASTERIDE:Fluid overload:0.13), (ENALAPRIL MALEATE:Fluid overload:0.37), (EPLERENONE:Fluid overload:0.13), (ESZOPICLONE:Fluid overload:0.4), (EVOLOCUMAB:Fluid overload:0.45), (FENOFIBRATE:Fluid overload:0.42), (FLECAINIDE:Fluid overload:0.45), (FLUTICASONE FUROATESALMETEROL XINAFOATE:Fluid overload:0.08), (GENTAMICIN:Fluid overload:0.23), (GLYBURIDE:Fluid overload:0.13), (HYDROCHLOROTHIAZIDELOSARTAN POTASSIUM:Fluid overload:0.04), (IDELALISIB:Fluid overload:0.2), (ILOPROST:Fluid overload:0.68), (INDAPAMIDEPERINDOPRIL:Fluid overload:0.09), (IPRATROPIUM:Fluid overload:0.49), (ITRACONAZOLE:Fluid overload:0.01), (LEDIPASVIRSOFOSBUVIR:Fluid overload:0.54), (LERCANIDIPINE:Fluid overload:0.95), (LEVALBUTEROL HYDROCHLORIDE:Fluid overload:0.33), (LINEZOLID:Fluid overload:0.13), (MIDODRINE:Fluid overload:0.24), (MOXIFLOXACIN HYDROCHLORIDE:Fluid overload:0.41), (NALOXONE HYDROCHLORIDEOXYCODONE HYDROCHLORIDE:Fluid overload:0.15), (NILOTINIB:Fluid overload:1.31), (NOREPINEPHRINE:Fluid overload:0.04), (OXCARBAZEPINE:Fluid overload:0.66), (PARICALCITOL:Fluid overload:0.06), (PENICILLIN:Fluid overload:0.15), (PHENOBARBITAL:Fluid overload:0.22), (PONATINIB:Fluid overload:0.59), (PRAMIPEXOLE DIHYDROCHLORIDE:Fluid overload:0.06), (PROBIOTICS NOS:Fluid overload:0.18), (PYRIDOXINE HYDROCHLORIDE:Fluid overload:0.04), (RABEPRAZOLE SODIUM:Fluid overload:0.05), (RIOCIGUAT:Fluid overload:0.92), (SULFAMETHOXAZOLE:Fluid overload:0.46), (THALIDOMIDE:Fluid overload:0.89), (VANCOMYCIN HYDROCHLORIDE:Fluid overload:0.68), (VITAMIN B:Fluid overload:0.18), (.ALPHA.1-PROTEINASE INHIBITOR HUMAN:Fluid replacement:0.13), (ACENOCOUMAROL:Fluid replacement:0.37), (ACETAMINOPHENOXYCODONEOXYCODONE HYDROCHLORIDE:Fluid replacement:0.5), (ADO-TRASTUZUMAB EMTANSINE:Fluid replacement:0.43), (AGALSIDASE BETA:Fluid replacement:0.12), (ALENDRONIC ACID:Fluid replacement:0.57), (AMISULPRIDE:Fluid replacement:0.45), (AMLODIPINEVALSARTAN:Fluid replacement:0.29), (ANASTROZOLE:Fluid replacement:1.15), (ANIDULAFUNGIN:Fluid replacement:0.17),(ARMODAFINIL:Fluid replacement:0.1), (BERAPROST:Fluid replacement:0.04), (BLINATUMOMAB:Fluid replacement:0.6), (BUDESONIDEFORMOTEROL FUMARATE:Fluid replacement:0.44), (BUSPIRONE HYDROCHLORIDE:Fluid replacement:0.31), (CALCITONIN:Fluid replacement:0.03), (CALCIUM CITRATE:Fluid replacement:0.29), (CANDESARTAN CILEXETILHYDROCHLOROTHIAZIDE:Fluid replacement:1.08), (CAPTOPRIL:Fluid replacement:0.31), (CARBIMAZOLE:Fluid replacement:0.01), (CARVEDILOL PHOSPHATE:Fluid replacement:0.48), (CHLOROTHIAZIDE:Fluid replacement:0.18), (CHLORTHALIDONE:Fluid replacement:0.31), (CLOBAZAM:Fluid replacement:0.21), (CLOMIPRAMINE HYDROCHLORIDE:Fluid replacement:0.26), (COLISTIMETHATE SODIUM:Fluid replacement:0.44), (COLISTIN:Fluid replacement:0.45), (CORTISONEHYDROCORTISONE:Fluid replacement:0.64), (CRIZOTINIB:Fluid replacement:0.02), (CYPROHEPTADINE:Fluid replacement:0.51), (DABIGATRAN:Fluid replacement:0.09), (DANAZOL:Fluid replacement:0.71), (DESLORATADINE:Fluid replacement:0.53), (DEXLANSOPRAZOLE:Fluid replacement:0.96), (DIMENHYDRINATE:Fluid replacement:0.51), (DIPYRONE:Fluid replacement:0.32), (DOFETILIDE:Fluid replacement:0.15), (DROPERIDOL:Fluid replacement:0.39), (DUTASTERIDE:Fluid replacement:0.5), (ECONAZOLE:Fluid replacement:0.28), (EDOXABAN TOSYLATE:Fluid replacement:0.83), (ELDECALCITOL:Fluid replacement:0.05), (ELETRIPTAN HYDROBROMIDE:Fluid replacement:0.39), (EPHEDRINE HYDROCHLORIDE:Fluid replacement:0.74), (EPIRUBICIN:Fluid replacement:0.32), (ERYTHROMYCIN:Fluid replacement:1.07), (ESMOLOL HYDROCHLORIDE:Fluid replacement:1.49), (ETIMICIN SULFATE:Fluid replacement:0.04), (ETORICOXIB:Fluid replacement:0.12), (EVOLOCUMAB:Fluid replacement:0.19), (EXEMESTANE:Fluid replacement:0.13), (EZETIMIBESIMVASTATIN:Fluid replacement:0.24), (FERROUS GLUCONATE:Fluid replacement:0.16), (FERROUS SULFATEGLYCINE:Fluid replacement:0.31), (FESOTERODINE FUMARATE:Fluid replacement:0.4), (FLUTICASONE FUROATESALMETEROL XINAFOATE:Fluid replacement:1.25), (FONDAPARINUX SODIUM:Fluid replacement:0.21), (FORMOTEROL FUMARATE DIHYDRATEMOMETASONE FUROATE:Fluid replacement:0.65), (FORMOTEROL FUMARATEMOMETASONE FUROATE:Fluid replacement:0.11), (FOSFOMYCIN:Fluid replacement:0.16), (FUSIDIC ACID:Fluid replacement:0.42), (GIMERACILOTERACILTEGAFUR:Fluid replacement:0.37), (GLYBURIDE:Fluid replacement:0.46), (GLYCOPYRRONIUMINDACATEROL:Fluid replacement:0.93), (HYDROCHLOROTHIAZIDEIRBESARTAN:Fluid replacement:0.78), (HYDROCHLOROTHIAZIDELOSARTAN POTASSIUM:Fluid replacement:0.44), (HYDROCHLOROTHIAZIDETELMISARTAN:Fluid replacement:0.1), (IBRITUMOMAB TIUXETAN:Fluid replacement:0.08), (ILOPROST:Fluid replacement:0.72), (IMIPENEM:Fluid replacement:0.93), (IMIPRAMINE HYDROCHLORIDE:Fluid replacement:0.05), (INDACATEROL:Fluid replacement:0.22), (INDAPAMIDEPERINDOPRIL:Fluid replacement:0.92), (IRINOTECAN HYDROCHLORIDE:Fluid replacement:0.21), (IXAZOMIB:Fluid replacement:0.13), (LAFUTIDINE:Fluid replacement:0.01), (LANDIOLOL HYDROCHLORIDE:Fluid replacement:0.13), (LEDIPASVIRSOFOSBUVIR:Fluid replacement:0.29), (LERCANIDIPINE:Fluid replacement:1.52), (LERCANIDIPINE HYDROCHLORIDE:Fluid replacement:0.55), (LEVALBUTEROL HYDROCHLORIDE:Fluid replacement:1.25), (LEVOLEUCOVORIN CALCIUM:Fluid replacement:0.18), (LITHIUM CARBONATE:Fluid replacement:0.81), (METAXALONE:Fluid replacement:0.12), (METHENAMINE HIPPURATE:Fluid replacement:0.52), (METHENOLONE ENANTHATE:Fluid replacement:0.77), (METHYLENE BLUE:Fluid replacement:0.12), (METHYLPREDNISOLONE ACETATE:Fluid replacement:0.25), (MUPIROCINMUPIROCIN CALCIUM:Fluid replacement:0.23), (NALOXONE:Fluid replacement:0.57), (NATEGLINIDE:Fluid replacement:0.16), (NILOTINIB:Fluid replacement:2.01), (NINTEDANIB:Fluid replacement:0.42), (NOREPINEPHRINE BITARTRATE:Fluid replacement:0.89), (NORTRIPTYLINE:Fluid replacement:0.5), (OLODATEROL HYDROCHLORIDETIOTROPIUM BROMIDE MONOHYDRATE:Fluid replacement:0.26), (OMEGA-3-ACID ETHYL ESTERS:Fluid replacement:0.51), (OXCARBAZEPINE:Fluid replacement:1.34), (OXYBUTYNIN CHLORIDE:Fluid replacement:0.21), (PANTOPRAZOLE MAGNESIUM:Fluid replacement:1.22), (PAROXETINE:Fluid replacement:0.53), (PAZOPANIB HYDROCHLORIDE:Fluid replacement:0.05), (PEGASPARGASE:Fluid replacement:0.17), (PENTAMIDINE ISETHIONATE:Fluid replacement:0.11), (PENTOSTATIN:Fluid replacement:0.29), (PRAMIPEXOLE:Fluid replacement:0.57), (PRANLUKAST:Fluid replacement:0.21), (PREDNISOLONE SODIUM PHOSPHATE:Fluid replacement:0.53), (PRIMIDONE:Fluid replacement:0.64), (PROTAMINE SULFATE:Fluid replacement:0.08), (QUETIAPINE:Fluid replacement:0.92), (RAMUCIRUMAB:Fluid replacement:0.25), (REGORAFENIB:Fluid replacement:0.03), (RETINOL:Fluid replacement:0.05), (ROFLUMILAST:Fluid replacement:0.69), (ROPINIROLE HYDROCHLORIDE:Fluid replacement:0.29), (SACUBITRILVALSARTAN:Fluid replacement:0.63), (SECUKINUMAB:Fluid replacement:0.29), (SUGAMMADEX SODIUM:Fluid replacement:0.34), (THIAMYLAL:Fluid replacement:0.27), (THROMBOMODULIN ALFA:Fluid replacement:0.3), (TIMOLOL:Fluid replacement:0.63), (TIZANIDINE:Fluid replacement:0.46), (TIZANIDINE HYDROCHLORIDE:Fluid replacement:0.41), (TRIAMCINOLONE ACETONIDE:Fluid replacement:0.53), (UMECLIDINIUM BROMIDEVILANTEROL TRIFENATATE:Fluid replacement:0.53), (VARENICLINE TARTRATE:Fluid replacement:0.48), (VITAMIN B:Fluid replacement:0.53), (VOGLIBOSE:Fluid replacement:0.03), (.ALPHA.-TOCOPHEROL ACETATE, DL-ASCORBIC ACIDCYANOCOBALAMINFLUORIDE IONFOLIC ACIDNIACINPYRIDOXINERIBOFLAVINTHIAMINEVITAMIN AVITAMIN D:Fluid retention:0.5), (ACEBUTOLOL HYDROCHLORIDE:Fluid retention:0.18), (ALBUTEROLIPRATROPIUM BROMIDE:Fluid retention:0.02), (ALEMTUZUMAB:Fluid retention:0.91), (AMINO ACIDSDEXTROSEELECTROLYTES NOS:Fluid retention:0.92), (AMINO ACIDSDEXTROSEELECTROLYTES NOSSOYBEAN OIL:Fluid retention:0.01), (AMISULPRIDE:Fluid retention:0.89), (AMLODIPINE BESYLATEHYDROCHLOROTHIAZIDEVALSARTAN:Fluid retention:0.87), (AMLODIPINE BESYLATEIRBESARTAN:Fluid retention:0.04), (AMLODIPINEPERINDOPRIL:Fluid retention:0.15), (AMMONIUM CHLORIDEDEXTROMETHORPHANSODIUM CITRATE:Fluid retention:0.41), (AMPICILLIN:Fluid retention:1.14), (ANAGRELIDE HYDROCHLORIDE:Fluid retention:0.37), (ANTIHISTAMINES NOS:Fluid retention:0.54), (ARIPIPRAZOLE LAUROXIL:Fluid retention:0.4), (ASPIRINCLOPIDOGREL:Fluid retention:0.1), (ASPIRINMAGNESIUM OXIDE:Fluid retention:0.01), (AZTREONAM:Fluid retention:0.12), (BACITRACIN:Fluid retention:0.76), (BAMIFYLLINE HYDROCHLORIDE:Fluid retention:0.01), (BELATACEPT:Fluid retention:0.11), (BENDAMUSTINE HYDROCHLORIDE:Fluid retention:0.52), (BEPRIDIL:Fluid retention:0.46), (BERAPROST:Fluid retention:0.48), (BETAHISTINE MESILATE:Fluid retention:0.1), (BETAMETHASONE DIPROPIONATECALCIPOTRIENE:Fluid retention:0.02), (BETAMETHASONECALCIPOTRIENE:Fluid retention:0.8), (BETHANECHOL CHLORIDE:Fluid retention:0.73), (BISMUTH SUBSALICYLATECALCIUM CARBONATE:Fluid retention:0.02), (BLINATUMOMAB:Fluid retention:0.73), (BOCEPREVIR:Fluid retention:0.02), (BOSUTINIB:Fluid retention:0), (BROMAZEPAM:Fluid retention:1.22), (CABAZITAXEL:Fluid retention:0.26), (CABOZANTINIB S-MALATE:Fluid retention:0.14), (CARBIMAZOLE:Fluid retention:0.6), (CARBOMER:Fluid retention:0.02), (CERITINIB:Fluid retention:0.36), (CETUXIMAB:Fluid retention:0.45), (CHLORAMBUCIL:Fluid retention:0.63), (CHOLESTEROL:Fluid retention:0.01), (CICLOPIROX:Fluid retention:0.01), (CILASTATIN SODIUMIMIPENEM:Fluid retention:1.5), (CLIKSTAR:Fluid retention:0.32), (CLOBAZAM:Fluid retention:0.21), (CLOMIPRAMINE HYDROCHLORIDE:Fluid retention:0.58), (COLISTIMETHATE SODIUM:Fluid retention:0.22), (CYPROHEPTADINE HYDROCHLORIDE:Fluid retention:0.38), (DABIGATRAN:Fluid retention:1.46), (DABRAFENIB:Fluid retention:0), (DABRAFENIB MESYLATE:Fluid retention:0.01), (DACLIZUMAB:Fluid retention:1.45), (DANAZOL:Fluid retention:1.18), (DEFLAZACORT:Fluid retention:1.69), (DESMOPRESSIN ACETATE:Fluid retention:0.12), (DESOXIMETASONE:Fluid retention:0), (DEXTROAMPHETAMINE:Fluid retention:0.28), (DIFLUNISAL:Fluid retention:0.16), (DIMETHICONESILICON DIOXIDESIMETHICONE:Fluid retention:0.22), (DIMETHYL SULFOXIDE:Fluid retention:0.06), (DIPYRONE:Fluid retention:0.89), (DOLASETRON MESYLATE:Fluid retention:0.59), (DROXIDOPA:Fluid retention:0.32), (ECONAZOLE:Fluid retention:0.9), (EDOXABAN TOSILATE MONOHYDRATE:Fluid retention:0.07), (ELDECALCITOL:Fluid retention:0.47), (ELTROMBOPAG OLAMINE:Fluid retention:0.48), (EMPAGLIFLOZINLINAGLIPTIN:Fluid retention:1.5), (ENALAPRIL MALEATEHYDROCHLOROTHIAZIDE:Fluid retention:0.12), (ERTAPENEM SODIUM:Fluid retention:0.06), (ESOMEPRAZOLE SODIUM:Fluid retention:0.18), (ESTROGENS:Fluid retention:0.67), (ETHINYL ESTRADIOLNORGESTREL:Fluid retention:0.5), (ETRETINATE:Fluid retention:0.44), (FENOTEROL HYDROBROMIDEIPRATROPIUM BROMIDE:Fluid retention:0.68), (FERRIC HYDROXIDE:Fluid retention:0.12), (FERROUS BISGLYCINATE:Fluid retention:0.2), (FERROUS SULFATEGLYCINE:Fluid retention:0.1), (FLURAZEPAMFLURAZEPAM HYDROCHLORIDE:Fluid retention:0.17), (FLUTICASONE FUROATESALMETEROL XINAFOATE:Fluid retention:1.79), (FONDAPARINUX:Fluid retention:0.25), (FORMOTEROL FUMARATEMOMETASONE FUROATE:Fluid retention:1.06), (FROVATRIPTAN SUCCINATE:Fluid retention:0.31), (GLYCOPYRROLATEINDACATEROL MALEATE:Fluid retention:0.62), (GRANISETRON HYDROCHLORIDE:Fluid retention:0.09), (GUANFACINE HYDROCHLORIDE:Fluid retention:0.13), (HYDROCORTISONE ACETATE:Fluid retention:0.89), (IBRITUMOMAB TIUXETAN:Fluid retention:0.29), (IMIGLUCERASE:Fluid retention:0.1), (INDAPAMIDEPERINDOPRIL:Fluid retention:1.61), (INFLIXIMAB-DYYB:Fluid retention:0.25), (INTERFERON:Fluid retention:0.75), (INTERFERON ALFA-2A:Fluid retention:0.61), (INTERFERON ALFA-2B:Fluid retention:0.52), (INTERFERON GAMMA-1B:Fluid retention:0.25), (IODINE:Fluid retention:0.64), (IRINOTECAN:Fluid retention:0.27), (IVABRADINE HYDROCHLORIDE:Fluid retention:0.33), (IXAZOMIB:Fluid retention:0.91), (LANREOTIDE:Fluid retention:0.66), (LANREOTIDE ACETATE:Fluid retention:1.39), (LAPATINIB DITOSYLATE:Fluid retention:0.67), (LENVATINIB:Fluid retention:0.18), (LEVOLEUCOVORIN CALCIUM:Fluid retention:0.08), (LEVOMEFOLATE CALCIUM:Fluid retention:0.88), (LEVOMILNACIPRAN HYDROCHLORIDE:Fluid retention:0.28), (LITHIUM:Fluid retention:0.79), (LUTETIUM LU-177:Fluid retention:0.27), (MAGNESIUM GLUCONATE:Fluid retention:0.36), (MEFENAMIC ACID:Fluid retention:0.57), (MEGESTROL:Fluid retention:0.61), (MEPOLIZUMAB:Fluid retention:0.89), (METFORMIN HYDROCHLORIDEROSIGLITAZONE MALEATE:Fluid retention:0.46), (METFORMIN HYDROCHLORIDESAXAGLIPTIN:Fluid retention:0.21), (METHENAMINE MANDELATE:Fluid retention:0.04), (METHENOLONE ENANTHATE:Fluid retention:0.55), (METRELEPTIN:Fluid retention:0.09), (NALOXONE:Fluid retention:0.35), (NALOXONE HYDROCHLORIDETILIDINE HYDROCHLORIDE:Fluid retention:0.66), (NIRAPARIB:Fluid retention:0.93), (NOREPINEPHRINE:Fluid retention:0.78), (OSELTAMIVIR:Fluid retention:0.4), (OXYBUTYNINOXYBUTYNIN CHLORIDE:Fluid retention:0.65), (PARATHYROID HORMONE:Fluid retention:0.32), (PEGINTERFERON ALFA-2B:Fluid retention:0.17), (PEGINTERFERON BETA-1A:Fluid retention:1.56), (PENICILLIN V:Fluid retention:0.62), (PENTOSTATIN:Fluid retention:0.27), (PILOCARPINE HYDROCHLORIDE:Fluid retention:0.3), (POLYETHYLENE GLYCOL 4000:Fluid retention:0.4), (PRANLUKAST:Fluid retention:0.28), (PREDNISOLONE SODIUM PHOSPHATE:Fluid retention:0.89), (PROTRIPTYLINE HYDROCHLORIDE:Fluid retention:0.71), (QUINACRINE:Fluid retention:0.16), (RADIATION THERAPY:Fluid retention:0.9), (ROSIGLITAZONE MALEATE:Fluid retention:0.51), (SIPULEUCEL-T:Fluid retention:0.51), (SODIUM CITRATE:Fluid retention:0.24), (TASIMELTEON:Fluid retention:0.11), (TAZOBACTAM:Fluid retention:0.15), (TEDUGLUTIDEWATER:Fluid retention:0.7), (TELOTRISTAT ETHYL:Fluid retention:0.57), (TEMSIROLIMUS:Fluid retention:0.02), (TESTOSTERONE UNDECANOATE:Fluid retention:1.05), (TINZAPARIN:Fluid retention:0.36), (TRAMETINIB DIMETHYL SULFOXIDE:Fluid retention:0.02), (TREPROSTINIL DIOLAMINE:Fluid retention:0.45), (TRIMIPRAMINE MALEATE:Fluid retention:0.1), (VARDENAFIL HYDROCHLORIDE TRIHYDRATE:Fluid retention:0.63), (ANASTROZOLE:Flushing:0.54),(ARMODAFINIL:Flushing:0.42), (BISACOD YL OR DOCUSATE SODIUM:Flushing:0.11), (BORTEZOMIB:Flushing:0.1), (CALCIUM ACETATE:Flushing:0.42), (DASATINIB:Flushing:0.24), (DEVICE:Flushing:0.33), (DICLOFEN AC SODIUM:Flushing:0.51), (DIGOXIN:Flushing:0.42), (ELETRIPTAN HYDROBROMIDE:Flushing:0.33), (ENOXAPARIN:Flushing:0.82), (ENOXAPARIN SODIUM:Flushing:0.01), (ESOMEPRAZOLE:Flushing:0.28), (EVOLOCUMAB:Flushing:0.58), (HYDROCHLOROTHIAZIDEVALSARTAN:Flushing:0.21), (IBRUTINIB:Flushing:1.13), (IN DOMETHACIN:Flushing:0.11), (ISOSORBIDE:Flushing:0.11), (LABETALOLLABETALOL HYDROCHLORIDE:Flushing:0.44), (LEVALBUTEROL HYDROCHLORIDE:Flushing:0.13), (METAXALONE:Flushing:0.26), (METHYLPHENIDATE HYDROCHLORIDE:Flushing:0.05), (MOXIFLOXACIN HYDROCHLORIDE:Flushing:0.03), (NILOTINIB:Flushing:0.32), (NITROFURANTOIN:Flushing: 0.22), (OMEGA-3-ACID ETHYL ESTERS:Flushing:0.03), (PANTOPRAZOLE MAGNESIUM:Flushing:0.14), (RIZATRIPTAN BENZOATE:Flushing:0.08), (SACUBITRILVALSARTAN:Flushing:0.41), (SUMATRIPTAN: Flushing:0.14), (SUMATRIPTANSUMATRIPTAN SUCCINATE:Flushing:0.34), (TERIFLUNOMIDE:Flushing:0.61), (THALIDOMIDE:Flushing:0.08), (TIZANIDINE HYDROCHLORIDE:Flushing:0.77), (TOCILIZUMAB:Flushing:0.37), (TOFACITINIB CITRATE:Flushing:0.02), (TRAMADOL HYDROCHLORIDE:Flushing:0.21), (ALBUTEROL SULFATEIPRATROPIUM BROMIDE:Foetal exposure during pregnancy:0.02), (ANASTROZOLE:Foetal exposure during pregnancy:0.09),(ARMODAFINIL:Foetal exposure during pregnancy:0.07), (ATORVASTATIN CALCIUM:Foetal exposure during pregnancy:0.87), (BUMETANIDE:Foetal exposure during pregnancy:0.1), (BUSPIRONE HYDROCHLORIDE:Foetal exposure during pregnancy:0.11), (CALCIUM CARBONATECHOLECALCIFEROL:Foetal exposure during pregnancy:0.66), (CALCIUMVITAMIN D:Foetal exposure during pregnancy:0.05), (CELECOXIB:Foetal exposure during pregnancy:0.52), (CIPROFLOXACIN HYDROCHLORIDE:Foetal exposure during pregnancy:0.1), (DESFLURANE:Foetal exposure during pregnancy:0.66), (DEXLANSOPRAZOLE:Foetal exposure during pregnancy:0.3), (DILTIAZEM HYDROCHLORIDE:Foetal exposure during pregnancy:0.13), (DULOXETINE:Foetal exposure during pregnancy:0.05), (ELETRIPTAN HYDROBROMIDE:Foetal exposure during pregnancy:0.13), (ESZOPICLONE:Foetal exposure during pregnancy:0.48), (EVOLOCUMAB:Foetal exposure during pregnancy:0.1), (FERROUS SULFATEGLYCINE:Foetal exposure during pregnancy:0.2), (IBRUTINIB:Foetal exposure during pregnancy:0.36), (ISOFLURANE:Foetal exposure during pregnancy:0.01), (LEVOFLOXACIN:Foetal exposure during pregnancy:0.45), (MELATONIN:Foetal exposure during pregnancy:0.25), (MELOXICAM:Foetal exposure during pregnancy:0.34), (METHYLPREDNISOLONE ACETATE:Foetal exposure during pregnancy:0.25), (MOXIFLOXACIN HYDROCHLORIDE:Foetal exposure during pregnancy:0.42), (RISPERIDONE:Foetal exposure during pregnancy:0.22), (ROSUVASTATIN CALCIUM:Foetal exposure during pregnancy:0.4), (SACUBITRILVALSARTAN:Foetal exposure during pregnancy:0.15), (TAMSULOSIN:Foetal exposure during pregnancy:0.39), (TELMISARTAN:Foetal exposure during pregnancy:0.16), (TEMAZEPAM:Foetal exposure during pregnancy:0.01), (TIZANIDINE HYDROCHLORIDE:Foetal exposure during pregnancy:0.39), (TOFACITINIB CITRATE:Foetal exposure during pregnancy:0.13), (VARENICLINE TARTRATE:Foetal exposure during pregnancy:0.5), (VERAPAMIL HYDROCHLORIDE:Foetal exposure during pregnancy:0.23), (ZOLPIDEMZOLPIDEM TARTRATE:Foetal exposure during pregnancy:0.48), (ALCOHOL:Folate deficiency:0.01), (AMISULPRIDE:Folate deficiency:0.21), (AMPHETAMINE ASPARTATEAMPHETAMINE SULFATEDEXTROAMPHETAMINE SACCHARATEDEXTROAMPHETAMINE SULFATE:Folate deficiency:0.4), (ANASTROZOLE:Folate deficiency:0.73), (BECLOMETHASONE DIPROPIONATE:Folate deficiency:0.07), (BROTIZOLAM:Folate deficiency:0.25), (BUDESONIDEFORMOTEROL FUMARATE:Folate deficiency:0.22), (BUPROPION:Folate deficiency:0.34), (CEPHALEXIN:Folate deficiency:0.93), (CILASTATIN SODIUMIMIPENEM:Folate deficiency:0.37), (CIPROFLOXACIN HYDROCHLORIDE:Folate deficiency:0.58), (CLOTRIMAZOLE:Folate deficiency:0.55), (CYANOCOBALAMINDEXPANTHENOLNIACINAMIDEPYRIDOXINE HYDROCHLORIDERIBOFLAVIN 5′-PHOSPHATE SODIUMTHIAMINE HYDROCHLORIDEVITAMIN B COMPLEX:Folate deficiency:0.07), (DACLIZUMAB:Folate deficiency:0.29), (DASATINIB:Folate deficiency:0.87), (DEXAMETHASONE ACETATE:Folate deficiency:0.06), (DICLOFENAC:Folate deficiency:1.15), (DIPHENHYDRAMINE:Folate deficiency:1.03), (DOXAZOSIN MESYLATE:Folate deficiency:0.35), (DOXYCYCLINE:Folate deficiency:0.38), (EPLERENONE:Folate deficiency:0.07), (EVOLOCUMAB:Folate deficiency:0.67), (FESOTERODINE FUMARATE:Folate deficiency:0.19), (FLUOXETINEFLUOXETINE HYDROCHLORIDE:Folate deficiency:0.22), (FLUTICASONE FUROATESALMETEROL XINAFOATE:Folate deficiency:0.09), (HEPARIN CALCIUM:Folate deficiency:0.15), (HYDROCHLOROTHIAZIDEIRBESARTAN:Folate deficiency:0.39), (HYDROCHLOROTHIAZIDELISINOPRIL:Folate deficiency:0.68), (HYDROMORPHONE:Folate deficiency:0.33), (HYDROXYZINE PAMOATE:Folate deficiency:0.36), (ILOPROST:Folate deficiency:0.01), (INDAPAMIDEPERINDOPRIL:Folate deficiency:0.63), (INFLUENZA VIRUS VACCINE:Folate deficiency:0.4), (IPRATROPIUM:Folate deficiency:0.19), (ISOSORBIDE:Folate deficiency:0.21), (LABETALOLLABETALOL HYDROCHLORIDE:Folate deficiency:0.15), (LERCANIDIPINE:Folate deficiency:1.22), (LERCANIDIPINE HYDROCHLORIDE:Folate deficiency:0.49), (LIDOCAINEPRILOCAINE:Folate deficiency:0.06), (MELATONIN:Folate deficiency:0.6), (METAXALONE:Folate deficiency:0.14), (METHYLPREDNISOLONE ACETATE:Folate deficiency:0.31), (MINOCYCLINEMINOCYCLINE HYDROCHLORIDE:Folate deficiency:0.06), (MODAFINIL:Folate deficiency:0.04), (NIACIN:Folate deficiency:0.03), (NORTRIPTYLINE:Folate deficiency:0.09), (NYSTATIN:Folate deficiency:0.57), (OMEGA-3-ACID ETHYL ESTERS:Folate deficiency:0.13), (OXYBUTYNIN CHLORIDE:Folate deficiency:0.06), (PANTOPRAZOLE MAGNESIUM:Folate deficiency:0.65), (PENICILLIN:Folate deficiency:0.13), (PHENOBARBITAL:Folate deficiency:0.44), (PIROXICAM:Folate deficiency:0.01), (PONATINIB:Folate deficiency:0.16), (PRAMIPEXOLE:Folate deficiency:0.72), (PROMETHAZINEPROMETHAZINE HYDROCHLORIDE:Folate deficiency:0.47), (PYRIDOXINE:Folate deficiency:0.06), (RALOXIFENE HYDROCHLORIDE:Folate deficiency:0.15), (RANOLAZINE:Folate deficiency:0.67), (RIZATRIPTAN BENZOATE:Folate deficiency:0.08), (SULFAMETHOXAZOLE:Folate deficiency:0.15), (TIMOLOL:Folate deficiency:0.22), (TOLTERODINE TARTRATE:Folate deficiency:0.01), (TRIAMCINOLONE ACETONIDE:Folate deficiency:0.43), (TRIAMTERENE:Folate deficiency:0.04), (VARENICLINE TARTRATE:Folate deficiency:0.83), (VINCRISTINE SULFATE:Folate deficiency:0.16), (ALBUTEROL SULFATE:Folliculitis:0), (CALCIUM CARBONATECHOLECALCIFEROL:Folliculitis:0.16), (DEXLANSOPRAZOLE:Folliculitis:0.2), (DIGOXIN:Folliculitis:0.17), (EPINEPHRINE:Folliculitis:0.23), (EVOLOCUMAB:Folliculitis: 0.23), (LEFLUNOMIDE:Folliculitis:0.07), (MELOXICAM:Folliculitis:0.16), (NEBIVOLOL:Folliculitis: 0.14), (PRAVASTATIN SODIUM:Folliculitis:0.08), (PYRAZINAMIDE:Folliculitis:0.62), (ROSUVASTATIN CALCIUM:Folliculitis:0.01), (VALSARTAN:Folliculitis:0.12), (WARFARIN SODIUM:Folliculitis:0.13), (DIVALPROEX SODIUM:Food allergy:0.19), (NAPROXEN:Food allergy:0.32), (TERIFLUNOMIDE:Food allergy:0.1), (VARENICLINE TARTRATE:Food allergy:0.43), (DEXLANSOPRAZOLE:Fracture:0.26), (DIVALPROEX SODIUM:Fracture:0.25), (ENALAPRIL:Fracture:0.44), (EVEROLIMUS:Fracture:0.19), (EVOL OCUMAB:Fracture:0.53), (FERROUS SULFATEFERROUS SULFATE, DRIED:Fracture:0.31), (IBRUTINIB:Fracture:0.9), (METRONIDAZOLE:Fracture:0.08), (NAPR OXEN SODIUM:Fracture:0.05), (RABEPRAZOLE SODIUM:Fracture:0.18), (SACUBITRILVALSARTAN:Fracture:0.1), (SPIRONOLACTONE: Fracture:0.57), (TAMSULOSIN HYDROCHLORIDE:Fracture:0.02), (TERIFLUNOMIDE:Fracture:0.24), (AMPHETAMINE ASPARTATEAMPHETAMINE SULFATEDEXTROAMPHETAMINE SACCHARATEDEXTROAMPHETAMINE SULFATE:Functional gastrointestinal disorder:0.28), (ANASTROZOLE:Functional gastrointestinal disorder:0.61), (BISOPROLOL FUMARATE:Functional gastrointestinal disorder:0.27), (BUMETANIDE:Functional gastrointestinal disorder:0.84), (CALCITRIOL:Functional gastrointestinal disorder:0.11), (CANDESARTAN:Functional gastrointestinal disorder:0.3), (CEPHALEXIN:Functional gastrointestinal disorder:0.1), (CLINDAMYCINCLINDAMYCIN PHOSPHATE:Functional gastrointestinal disorder:0.16), (COLCHICINE:Functional gastrointestinal disorder:0.38), (CORTISONEHYDROCORTISONE:Functional gastrointestinal disorder:0.07), (DASATINIB:Functional gastrointestinal disorder:0.19), (DIPHENHYDRAMINE:Functional gastrointestinal disorder:0.75), (ELETRIPTAN HYDROBROMIDE:Functional gastrointestinal disorder:0.04), (ENALAPRIL:Functional gastrointestinal disorder:1.39), (ESZOPICLONE:Functional gastrointestinal disorder:0.56), (EVOLOCUMAB:Functional gastrointestinal disorder:0.72), (FLUTICASONE:Functional gastrointestinal disorder:0.04), (HYDROXYZINE PAMOATE:Functional gastrointestinal disorder:0.03), (IBRUTINIB:Functional gastrointestinal disorder:1.44), (LABETALOLLABETALOL HYDROCHLORIDE:Functional gastrointestinal disorder:0.06), (LISDEXAMFETAMINE DIMESYLATE:Functional gastrointestinal disorder:0.02), (NILOTINIB:Functional gastrointestinal disorder:0.12), (NITROFURANTOIN:Functional gastrointestinal disorder:0.23), (NYSTATIN:Functional gastrointestinal disorder:0.04), (OLANZAPINE:Functional gastrointestinal disorder:0.36), (OMEGA-3-ACID ETHYL ESTERS:Functional gastrointestinal disorder:0.04), (OXYGEN:Functional gastrointestinal disorder:0.42), (PANTOPRAZOLE MAGNESIUM:Functional gastrointestinal disorder:0.15), (PEGFILGRASTIM:Functional gastrointestinal disorder:0.19), (PRIMIDONE:Functional gastrointestinal disorder:0.15), (RANOLAZINE:Functional gastrointestinal disorder:0.68), (SACUBITRILVALSARTAN:Functional gastrointestinal disorder:0.52), (SUCRALFATE:Functional gastrointestinal disorder:0.03), (TOCILIZUMAB:Functional gastrointestinal disorder:0.42), (TORSEMIDE:Functional gastrointestinal disorder:0.11), (UMECLIDINIUM BROMIDEVILANTEROL TRIFENATATE:Functional gastrointestinal disorder:0.06), (VARENICLINE TARTRATE:Functional gastrointestinal disorder:0.34), (VITAMIN B:Functional gastrointestinal disorder:0.36), (.ALPHA.1-PROTEINASE INHIBITOR HUMAN:Fungal infection:1.39), (ABOBOTULINUMTOXINA:Fungal infection:0.15), (ACETAMINOPHENCAFFEINE:Fungal infection:0.13), (ACETAMINOPHENCAFFEINEOPIUM:Fungal infection:0.05), (AGALSIDASE BETA:Fungal infection:0.78), (ALFUZOSIN HYDROCHLORIDE:Fungal infection:0.46), (ALUMINUM HYDROXIDEMAGNESIUM OXIDEOXETHAZAINE:Fungal infection:0.2), (AMANTADINE HYDROCHLORIDE:Fungal infection:0.32), (AMLODIPINE BESYLATEBENAZEPRIL HYDROCHLORIDE:Fungal infection:0.08), (AMLODIPINE BESYLATEHYDROCHLOROTHIAZIDEOLMESARTAN MEDOXOMIL:Fungal infection:0.22), (AMLODIPINE BESYLATEHYDROCHLOROTHIAZIDEVALSARTAN:Fungal infection:0.07), (AMLODIPINE BESYLATEOLMESARTAN MEDOXOMIL:Fungal infection:0.19), (AMLODIPINE BESYLATEVALSARTAN:Fungal infection:0.25), (AMLODIPINEVALSARTAN:Fungal infection:0.63), (AMMONIUM GLYCYRRHIZATECYSTEINE HYDROCHLORIDEGLYCINE:Fungal infection:0.14), (ANTIHISTAMINES NOS:Fungal infection:0.6), (ASPARAGINASE ERWINIA CHRYSANTHEMI:Fungal infection:0.12), (ATROPINEDIPHENOXYLATE:Fungal infection:0.11), (AZILSARTAN KAMEDOXOMIL:Fungal infection:0.38), (BALSALAZIDE DISODIUM:Fungal infection:0.26), (BELATACEPT:Fungal infection:0.04), (BENAZEPRILBENAZEPRIL HYDROCHLORIDE:Fungal infection:1.22), (BETAMIPRONPANIPENEM:Fungal infection:0.5), (BILASTINE:Fungal infection:0.1), (BRIMONIDINE TARTRATETIMOLOL MALEATE:Fungal infection:0.13), (BRINZOLAMIDE:Fungal infection:0.68), (BUPIVACAINE HYDROCHLORIDE:Fungal infection:0.07), (BUPRENORPHINE HYDROCHLORIDENALOXONE HYDROCHLORIDE:Fungal infection:0.79), (CALCITONIN:Fungal infection:0.77), (CALCIUM CARBONATEERGOCALCIFEROLRETINOL:Fungal infection:0.16), (CALCIUM POLYCARBOPHIL:Fungal infection:0.22), (CALCIUMMAGNESIUM:Fungal infection:0.74), (CALCIUMMAGNESIUMZINC:Fungal infection:0.45), (CANAGLIFLOZINMETFORMIN HYDROCHLORIDE:Fungal infection:1.06), (CARBIMAZOLE:Fungal infection:0.17), (CEFOXITINCEFOXITIN SODIUM:Fungal infection:0.12), (CEFPIROME SULFATE:Fungal infection:0.39), (CEFPROZIL:Fungal infection:0.51), (CHLORDIAZEPOXIDE HYDROCHLORIDECLIDINIUM BROMIDE:Fungal infection:0.2), (CHLOROTHIAZIDE:Fungal infection:0.25), (CLADRIBINE:Fungal infection:0.3), (CLEMASTINE FUMARATE:Fungal infection:0.16), (CODEINE PHOSPHATEGUAIFENESIN:Fungal infection:0.45), (CORTICOTROPIN:Fungal infection:0.58), (CYCLOPHOSPHAMIDECYTARABINEDEXAMETHASONEDOXORUBI CINMETHOTREXATEVINCRISTINE:Fungal infection:0.21), (CYPROHEPTADINE HYDROCHLORIDE:Fungal infection:0.69), (DABRAFENIB:Fungal infection:0.71), (DANTROLENE SODIUM:Fungal infection:0.09), (DASABUVIROMBITASVIRPARITAPREVIRRITONAVIR:Fungal infection:0.03), (DEFIBROTIDE (BOVINE):Fungal infection:0.35), (DEFLAZACORT:Fungal infection:0.61), (DESMOPRESSIN:Fungal infection:0.01), (DESONIDE:Fungal infection:0.97), (DIGITOXIN:Fungal infection:0.21), (DIMETHICONESILICON DIOXIDESIMETHICONE:Fungal infection:0.58), (DIPYRIDAMOLE:Fungal infection:0.61), (DOBUTAMINE HYDROCHLORIDE:Fungal infection:0.06), (DORZOLAMIDE HYDROCHLORIDE:Fungal infection:0.1), (DUTASTERIDE:Fungal infection:1.62), (ELOTUZUMAB:Fungal infection:0.81), (EMPAGLIFLOZINLINAGLIPTIN:Fungal infection:1.04), (EPINASTINE HYDROCHLORIDE:Fungal infection:0.07), (ESOMEPRAZOLE MAGNESIUMNAPROXEN:Fungal infection:0.17), (ESTRADIOLNORETHINDRONE ACETATE:Fungal infection:0.3), (ETOPOSIDE PHOSPHATE:Fungal infection:0.77), (ETORICOXIB:Fungal infection:0.59), (FENOFIBRIC ACID:Fungal infection:0.47), (FERROUS FUMARATE:Fungal infection:0.62), (FERROUS SULFATEGLYCINE:Fungal infection:0.06), (FEXOFENADINEPSEUDOEPHEDRINE:Fungal infection:0.32), (FLUOROMETHOLONE:Fungal infection:0.01), (FLURAZEPAMFLURAZEPAM HYDROCHLORIDE:Fungal infection:0.03), (FLUVOXAMINE MALEATE:Fungal infection:0.38), (FONDAPARINUX:Fungal infection:0.09), (FORMOTEROL FUMARATEMOMETASONE FUROATE:Fungal infection:1.22), (FOSPHENYTOINFOSPHENYTOIN SODIUM:Fungal infection:0.34), (GARENOXACIN:Fungal infection:0.01), (GIMERACILOTERACILTEGAFUR:Fungal infection:0.6), (GOSERELIN ACETATE:Fungal infection:0.06), (GUANFACINE:Fungal infection:0.04), (GUANFACINE HYDROCHLORIDE:Fungal infection:0.16), (HYDROCHLOROTHIAZIDEIRBESARTAN:Fungal infection:1.32), (HYDROCODONE BITARTRATEIBUPROFEN:Fungal infection:0.36), (HYDROXYCHLOROQUINE DIPHOSPHATE:Fungal infection:0.34), (IBRITUMOMAB TIUXETAN:Fungal infection:1.11), (ILOPROST:Fungal infection:0.5), (IMATINIB MESYLATE:Fungal infection:1.44), (IMIQUIMOD:Fungal infection:0.22), (INDAPAMIDEPERINDOPRIL:Fungal infection:1.67), (INTERFERON ALFA-2A:Fungal infection:0.55), (INTERFERON GAMMA-1B:Fungal infection:1.24), (IODINE:Fungal infection:0.07), (IRINOTECAN HYDROCHLORIDE:Fungal infection:1.07), (IXAZOMIB:Fungal infection:0.25), (KETOROLACKETOROLAC TROMETHAMINE:Fungal infection:0.86), (LABETALOL HYDROCHLORIDE:Fungal infection:1.01), (LANREOTIDE ACETATE:Fungal infection:0.54), (LAPATINIB DITOSYLATE:Fungal infection:0.33), (LERCANIDIPINE HYDROCHLORIDE:Fungal infection:1.15), (LEVALBUTEROL:Fungal infection:0.5), (LEVOMEFOLATE CALCIUM:Fungal infection:0.14), (LISDEXAMFETAMINE DIMESYLATE:Fungal infection:1.08), (LUMACAFTOR:Fungal infection:0.27), (MAGNESIUM ASPARTATEPOTASSIUM ASPARTATE:Fungal infection:0.17), (MEDROXYPROGESTERONE:Fungal infection:0.34), (MELPERONE:Fungal infection:0.09), (MEPOLIZUMAB:Fungal infection:0.98), (METFORMIN HYDROCHLORIDEVILDAGLIPTIN:Fungal infection:0.33), (MOGAMULIZUMAB:Fungal infection:0.09), (NALOXONE HYDROCHLORIDETILIDINE HYDROCHLORIDE:Fungal infection:0.29), (NICORANDIL:Fungal infection:0.24), (NILOTINIB:Fungal infection:2.38), (NINTEDANIB:Fungal infection:0.9), (NIRAPARIB:Fungal infection:0.38), (NOREPINEPHRINE:Fungal infection:1.19), (OLMESARTAN:Fungal infection:0.26), (OLODATEROL HYDROCHLORIDETIOTROPIUM BROMIDE MONOHYDRATE:Fungal infection:0.85), (OLOPATADINE:Fungal infection:1.17), (ORPHENADRINE:Fungal infection:0.3), (PANITUMUMAB:Fungal infection:0.38), (PANTOPRAZOLE MAGNESIUM:Fungal infection:2.06), (PATIROMER:Fungal infection:0.22), (PAZOPANIB HYDROCHLORIDE:Fungal infection:1.23), (PEGINTERFERON BETA-1A:Fungal infection:1.12), (PENICILLIN G:Fungal infection:0.42), (PHENYTOIN SODIUM:Fungal infection:0.96), (PILOCARPINE:Fungal infection:0.26), (PIROXICAM:Fungal infection:0.55), (PITAVASTATIN CALCIUM:Fungal infection:0.89), (PLERIXAFOR:Fungal infection:0.08), (PNEUMOCOCCAL VACCINE, POLYVALENT 23:Fungal infection:0.22), (PRAZOSIN HYDROCHLORIDE:Fungal infection:0.55), (PROPAFENONE HYDROCHLORIDE:Fungal infection:0.06), (QUINAPRIL:Fungal infection:0.37), (QUINAPRILQUINAPRIL HYDROCHLORIDE:Fungal infection:0.03), (RADIATION THERAPY:Fungal infection:0.96), (RALOXIFENE HYDROCHLORIDE:Fungal infection:0.88), (RAMUCIRUMAB:Fungal infection:0.3), (RIVASTIGMINE:Fungal infection:0.54), (ROFLUMILAST:Fungal infection:1.72), (SACUBITRILVALSARTAN:Fungal infection:0.42), (SILVER SULFADIAZINE:Fungal infection:0.45), (SODIUM CITRATE:Fungal infection:0.22), (SODIUM POLYSTYRENE SULFONATE:Fungal infection:0.18), (SUFENTANIL CITRATE:Fungal infection:0.55), (TAMOXIFEN CITRATE:Fungal infection:0.04), (TEDUGLUTIDEWATER:Fungal infection:0.51), (TERAZOSINTERAZOSIN HYDROCHLORIDE:Fungal infection:0.3), (TESTOSTERONE UNDECANOATE:Fungal infection:0.06), (TETRACYCLINE:Fungal infection:1.36), (TIOTROPIUM BROMIDE:Fungal infection:0.31), (TISAGENLECLEUCEL:Fungal infection:0.18), (TOLTERODINE:Fungal infection:0.29), (TRAMETINIB:Fungal infection:0.64), (TRANDOLAPRIL:Fungal infection:0.23), (TROSPIUM CHLORIDE:Fungal infection:0.58), (VINDESINE SULFATE:Fungal infection:0.3), (VINORELBINE TARTRATE:Fungal infection:0.38), (VINORELBINEVINORELBINE TARTRATE:Fungal infection:0.2), (ZILEUTON:Fungal infection:0.43), (ZIPRASIDONE HYDROCHLORIDEZIPRASIDONE MESYLATE:Fungal infection:0.17), (ZIV-AFLIBERCEPT: Fungal infection:0.12), (ANASTROZOLE:Fungal skin infection:0.36), (BUMETANIDE:Fungal skin infection:0.57), (BUSPIRONE HYDROCHLORIDE:Fungal skin infection:0.07), (CALCIUM CARBONATECHOLECALCIFEROL:Fungal skin infection:0.77), (CERTOLIZUMAB PEGOL:Fungal skin infection:0.29), (DEXLANSOPRAZOLE:Fungal skin infection:0.49), (ENALAPRIL:Fungal skin infection:0.4), (EPINEPHRINE:Fungal skin infection:0.4), (EVOLOCUMAB:Fungal skin infection:0.14), (IBRUTINIB:Fungal skin infection:0.96), (LOVASTATIN:Fungal skin infection:0.08), (NEBIVOLOL:Fungal skin infection:0.12), (OXYGEN:Fungal skin infection:0.06), (PEGFILGRASTIM:Fungal skin infection:0.12), (PRAVASTATIN SODIUM:Fungal skin infection:0.1), (RANOLAZINE:Fungal skin infection:0.02), (SACUBITRILVALSARTAN:Fungal skin infection:0.6), (SERTRALINE HYDROCHLORIDE:Fungal skin infection:0.89), (TERIFLUNOMIDE:Fungal skin infection:0.73), (TIZANIDINE HYDROCHLORIDE:Fungal skin infection:0.24), (VERAPAMIL HYDROCHLORIDE:Fungal skin infection:0.21), (.ALPHA.1-PROTEINASE INHIBITOR HUMAN:Gait disturbance:0.28), (ABOBOTULINUMTOXINA:Gait disturbance:0.25), (ACETAMINOPHENCAFFEINE:Gait disturbance:0.03), (ACETAMINOPHENMETHOCARBAMOL:Gait disturbance:0.05), (ACETAMINOPHENOXYCODONE HYDROCHLORIDEOXYCODONE TEREPHTHALATE:Gait disturbance:1.19), (ACETAMINOPHENTRAMADOL:Gait disturbance:0.64), (ACITRETIN:Gait disturbance:0.23), (AGALSIDASE BETA:Gait disturbance:0.59), (ALMOTRIPTAN MALATE:Gait disturbance:0.11), (AMILORIDE HYDROCHLORIDEHYDROCHLOROTHIAZIDE:Gait disturbance:0.3), (AMINO ACIDSDEXTROSEELECTROLYTES NOS:Gait disturbance:0.16), (AMLODIPINE BESYLATEATORVASTATIN CALCIUM:Gait disturbance:0.47), (AMLODIPINEVALSARTAN:Gait disturbance:0.69), (AMMONIUM CHLORIDEDEXTROMETHORPHANSODIUM CITRATE:Gait disturbance:0.07), (ARIPIPRAZOLE LAUROXIL:Gait disturbance:0.39), (ASPIRINDIHYDROXYALUMINUM AMINOACETATE ANHYDROUSMAGNESIUM CARBONATE:Gait disturbance:0.1), (ATENOLOLCHLORTHALIDONE:Gait disturbance:0.11), (ATROPINE SULFATEHYOSCYAMINE SULFATEPHENOBARBITALSCOPOLAMINE HYDROBROMIDE:Gait disturbance:0.1), (ATROPINEDIPHENOXYLATE:Gait disturbance:0.08), (AVANAFIL:Gait disturbance:0.21), (AXITINIB:Gait disturbance:0.06), (AZELASTINE HYDROCHLORIDEFLUTICASONE PROPIONATE:Gait disturbance:0.29), (BACITRACIN:Gait disturbance:0.53), (BALSALAZIDE DISODIUM:Gait disturbance:0.39), (BETAMETHASONECALCIPOTRIENE:Gait disturbance:0.29), (BIFIDOBACTERIUM ANIMALIS SUBSP. LACTIS:Gait disturbance:0.03), (BOTULINUM TOXIN TYPE A:Gait disturbance:0), (BRINZOLAMIDE:Gait disturbance:1), (BUPRENORPHINE HYDROCHLORIDE:Gait disturbance:1.02), (BUTYLSCOPOLAMINE BROMIDE:Gait disturbance:0.12), (CALCIPOTRIENE:Gait disturbance:0.54), (CANAKINUMAB:Gait disturbance:0.44), (CAPSAICIN:Gait disturbance:0.41), (CARBOMER:Gait disturbance:0.24), (CARBOXYMETHYLCELLULOSE SODIUM:Gait disturbance:0.16), (CEFAZOLIN SODIUM:Gait disturbance:0.05), (CERIVASTATIN SODIUM:Gait disturbance:0.24), (CHLORHEXIDINE:Gait disturbance:0.59), (CIPROFIBRATE:Gait disturbance:0.04), (CLIKSTAR:Gait disturbance:0.07), (CODEINE PHOSPHATE:Gait disturbance:0.09), (CODEINE PHOSPHATEGUAIFENESIN:Gait disturbance:0.64), (DABIGATRAN:Gait disturbance:0.21), (DALTEPARIN:Gait disturbance:0.58), (DANAZOL:Gait disturbance:0.43), (DAPAGLIFLOZIN PROPANEDIOLMETFORMIN HYDROCHLORIDE:Gait disturbance:0.04), (DASABUVIROMBITASVIRPARITAPREVIRRITONAVIR:Gait disturbance:0.11), (DASATINIB:Gait disturbance:1.15), (DIPYRIDAMOLE:Gait disturbance:0.21), (DIPYRONE:Gait disturbance:0.54), (DIPYRONEPROMETHAZINE HYDROCHLORIDE:Gait disturbance:0.05), (DOFETILIDE:Gait disturbance:0.92), (DOLASETRON MESYLATE:Gait disturbance:0.06), (DROSPIRENONEETHINYL ESTRADIOLLEVOMEFOLATE CALCIUM:Gait disturbance:0.21), (ECONAZOLE:Gait disturbance:0.3), (EDOXABAN TOSYLATE:Gait disturbance:0.66), (EMPAGLIFLOZINLINAGLIPTIN:Gait disturbance:0.81), (ERYTHROPOIETIN:Gait disturbance:0.96), (ESOMEPRAZOLE MAGNESIUMNAPROXEN:Gait disturbance:0.66), (ESTRADIOL BENZOATE:Gait disturbance:0.14), (ETHINYL ESTRADIOLGESTODENE:Gait disturbance:0.08), (ETORICOXIB:Gait disturbance:0.88), (EVEROLIMUS:Gait disturbance:0.73), (FERROUS BISGLYCINATE:Gait disturbance:0.31), (FILGRASTIM:Gait disturbance:0.15), (FLURAZEPAMFLURAZEPAM HYDROCHLORIDE:Gait disturbance:0.25), (FLUTICASONE FUROATESALMETEROL XINAFOATE:Gait disturbance:0.7), (FORMOTEROL:Gait disturbance:0.35), (FORMOTEROL FUMARATEMOMETASONE FUROATE:Gait disturbance:0.33), (FUSIDIC ACID:Gait disturbance:0.9), (GLYCOPYRROLATEINDACATEROL MALEATE:Gait disturbance:0.01), (GLYCOPYRRONIUMANDACATEROL:Gait disturbance:0.74), (GUANFACINE:Gait disturbance:0.77), (GUANFACINE HYDROCHLORIDE:Gait disturbance:0.18), (HYALURONIC ACID:Gait disturbance:0.07), (HYDROCHLOROTHIAZIDETELMISARTAN:Gait disturbance:1), (HYDROXYCHLOROQUINE DIPHOSPHATE:Gait disturbance:0.86), (ILOPROST:Gait disturbance:0.08), (INDAPAMIDEPERINDOPRIL:Gait disturbance:0.72), (INFLIXIMAB-DYYB:Gait disturbance:0.06), (LENALIDOMIDE:Gait disturbance:0.7), (LERCANIDIPINE HYDROCHLORIDE:Gait disturbance:0.83), (LEVALBUTEROL TARTRATE:Gait disturbance:0.03), (LIDOCAINE HYDROCHLORIDE:Gait disturbance:1.19), (LOMITAPIDE MESYLATE:Gait disturbance:1.11), (MEPOLIZUMAB:Gait disturbance:0.13), (METRELEPTIN:Gait disturbance:0.01), (MOEXIPRIL HYDROCHLORIDE:Gait disturbance:0.11), (MOXIFLOXACIN:Gait disturbance:0.5), (NADROPARIN CALCIUM:Gait disturbance:0.23), (NALOXONE:Gait disturbance:0.18), (NALOXONE HYDROCHLORIDEOXYCODONE HYDROCHLORIDE:Gait disturbance:1.3), (NALOXONE HYDROCHLORIDETILIDINE HYDROCHLORIDE:Gait disturbance:0.15), (NEPAFENAC:Gait disturbance:0.1), (NIRAPARIB:Gait disturbance:0.03), (OLODATEROL HYDROCHLORIDETIOTROPIUM BROMIDE MONOHYDRATE:Gait disturbance:0.12), (OPIPRAMOL:Gait disturbance:0.14), (OSELTAMIVIR PHOSPHATE:Gait disturbance:0.35), (OSPEMIFENE:Gait disturbance:0.57), (PAMIDRONATE DISODIUM:Gait disturbance:0.48), (PARATHYROID HORMONE:Gait disturbance:0.06), (PATIROMER:Gait disturbance:0.25), (PENICILLIN G:Gait disturbance:0.15), (PENICILLIN V:Gait disturbance:0.09), (PHENTERMINE HYDROCHLORIDETOPIRAMATE:Gait disturbance:0.03), (PHENYTOIN SODIUM:Gait disturbance:0.49), (PIMECROLIMUS:Gait disturbance:0.51), (PIRFENIDONE:Gait disturbance:0), (PONATINIB:Gait disturbance:0.71), (PRAMLINTIDE ACETATE:Gait disturbance:0.04), (PROTRIPTYLINE HYDROCHLORIDE:Gait disturbance:1.03), (PYRAZINAMIDE:Gait disturbance:0.69), (RALTEGRAVIR POTASSIUM:Gait disturbance:0.13), (ROFECOXIB:Gait disturbance:0.1), (ROFLUMILAST:Gait disturbance:1.31), (SACUBITRILVALSARTAN:Gait disturbance:0.78), (SALMETEROL:Gait disturbance:0.31), (SECUKINUMAB:Gait disturbance:0.92), (SELEGILINE:Gait disturbance:0.89), (SIBUTRAMINE HYDROCHLORIDE:Gait disturbance:0.4), (SOTALOL HYDROCHLORIDE:Gait disturbance:0.1), (SUNITINIB MALATE:Gait disturbance:0.51), (TASIMELTEON:Gait disturbance:0.22), (TEDUGLUTIDEWATER:Gait disturbance:0.09), (TERBUTALINE:Gait disturbance:0.18), (TERBUTALINE SULFATE:Gait disturbance:1.63), (TESTOSTERONE UNDECANOATE:Gait disturbance:0.29), (THALIDOMIDE:Gait disturbance:0.3), (TIBOLONE:Gait disturbance:0.11), (TRIMIPRAMINE MALEATE:Gait disturbance:0.65), (UMECLIDINIUM BROMIDE:Gait disturbance:0.38), (UMECLIDINIUM BROMIDEVILANTEROL TRIFENATATE:Gait disturbance:0.8), (VALGANCICLOVIR HYDROCHLORIDE:Gait disturbance:0.47), (ZILEUTON:Gait disturbance:0.18), (LEUCOVORIN CALCIUM:Gallbladder cancer:0.13), (RIFAMPIN:Gallbladder disorder:0.67), (BRIVARACETAM:Ganglioglioma:0.2), (CITALOPRAM HYDROBROMIDE:Gastric bypass:0.34), (DALFAMPRIDINE:Gastric bypass:0.11), (MELATONIN:Gastric bypass:0.03), (SIMVASTATIN:Gastric bypass:0.06), (TERIFLUNOMIDE:Gastric bypass:0.05), (VARENICLINE TARTRATE:Gastric bypass:0.33), (WARFARIN:Gastric bypass:0.15), (ABIRATERONE:Gastric cancer:0.04), (ABIRATERONE ACETATE:Gastric cancer:0.17), (ALEMTUZUMAB:Gastric cancer:0.01), (ALFACALCIDOL:Gastric cancer:1.12), (AMIODARONE:Gastric cancer:1.15), (AMIODARONE HYDROCHLORIDE:Gastric cancer:0.77), (ASPIRIN LYSINE:Gastric cancer:0.36), (ATROPINE SULFATEDIPHENOXYLATE HYDROCHLORIDE:Gastric cancer:0.1), (AZITHROMYCIN ANHYDROUS:Gastric cancer:0.05), (BENAZEPRIL HYDROCHLORIDE:Gastric cancer:0.12), (BORTEZOMIB:Gastric cancer:0.16), (BUDESONIDE:Gastric cancer:1.07), (BUMETANIDE:Gastric cancer:0.72), (BUPROPION:Gastric cancer:0.34), (BUPROPION HYDROCHLORIDE:Gastric cancer:0.47), (CALCITONIN SALMON:Gastric cancer:0.65), (CALCITRIOL:Gastric cancer:0.86), (CARFILZOMIB:Gastric cancer:0.19), (CERTOLIZUMAB PEGOL:Gastric cancer:1.06), (CHOLESTYRAMINE:Gastric cancer:0.5), (CLINDAMYCIN:Gastric cancer:0.15), (CLINDAMYCINCLINDAMYCIN PHOSPHATE:Gastric cancer:0.93), (CLOTRIMAZOLE:Gastric cancer:0.25), (CLOZAPINE:Gastric cancer:0.4), (CYANOCOBALAMINDEXPANTHENOLNIACINAMIDEPYRIDOXINE HYDROCHLORIDERIBOFLAVIN 5′-PHOSPHATE SODIUMTHIAMINE HYDROCHLORIDEVITAMIN B COMPLEX:Gastric cancer:0.07), (DABRAFENIB:Gastric cancer:0.95), (DASATINIB:Gastric cancer:1.13), (DEVICE:Gastric cancer:0.08), (EPINASTINE HYDROCHLORIDE:Gastric cancer:0.45), (ERIBULIN:Gastric cancer:0.81), (ERIBULIN MESYLATE:Gastric cancer:0.13), (ESZOPICLONE:Gastric cancer:0.89), (ETRETINATE:Gastric cancer:0.12), (EVOLOCUMAB:Gastric cancer:0.06), (EZETIMIBESIMVASTATIN:Gastric cancer:0.16), (FULVESTRANT:Gastric cancer:1.22), (GEFITINIB:Gastric cancer:0.71), (ILOPROST:Gastric cancer:0.01), (INDAPAMIDE:Gastric cancer:0.13), (IPRATROPIUM:Gastric cancer:0.03), (ISOSORBIDE:Gastric cancer:0.43), (LENVATINIB:Gastric cancer:0.29), (LERCANIDIPINE:Gastric cancer:0.34), (LERCANIDIPINE HYDROCHLORIDE:Gastric cancer:0.14), (METFORMIN HYDROCHLORIDEVILDAGLIPTIN:Gastric cancer:0.11), (METHADONE HYDROCHLORIDE:Gastric cancer:0.02), (MINOCYCLINE HYDROCHLORIDE:Gastric cancer:0.32), (MOXIFLOXACIN HYDROCHLORIDE:Gastric cancer:0.67), (NEBIVOLOL:Gastric cancer:0.22), (OMEGA-3-ACID ETHYL ESTERS:Gastric cancer:0.35), (OSIMERTINIB:Gastric cancer:0.3), (OXCARBAZEPINE:Gastric cancer:0.02), (QUETIAPINE FUMARATE:Gastric cancer:0.01), (RANOLAZINE:Gastric cancer:0.49), (ROFLUMILAST:Gastric cancer:0.42), (SEVELAMER HYDROCHLORIDE:Gastric cancer:0.05), (SIPULEUCEL-T:Gastric cancer:0.08), (SITAGLIPTIN:Gastric cancer:0.45), (SOTALOL:Gastric cancer:0.65), (SULFAMETHOXAZOLE:Gastric cancer:0.51), (TAMOXIFEN CITRATE:Gastric cancer:0.41), (TEMOZOLOMIDE:Gastric cancer:0.18), (TIMOLOL:Gastric cancer:0.49), (TINZAPARIN SODIUM:Gastric cancer:0.78), (TOFACITINIB CITRATE:Gastric cancer:0.08), (TRAMETINIB:Gastric cancer:1.06), (TRICHLORMETHIAZIDE:Gastric cancer:0.21), (VEMURAFENIB:Gastric cancer:0.73), (VINORELBINE TARTRATE:Gastric cancer:0.66), (.ALPHA.-TOCOPHEROL ACETATE, DL-ASCORBIC ACIDCYANOCOBALAMINFLUORIDE IONFOLIC ACIDNIACINPYRIDOXINERIBOFLAVINTHIAMINEVITAMIN AVITAMIN D:Gastric disorder:0.47), (.ALPHA.1-PROTEINASE INHIBITOR HUMAN:Gastric disorder:0.67), (ABOBOTULINUMTOXINA:Gastric disorder:0.15), (ACEBUTOLOL:Gastric disorder:0.15), (ACETAMINOPHENCAFFEINE:Gastric disorder:0.48), (ACETAMINOPHENCAFFEINEOPIUM:Gastric disorder:0.77), (ACETAMINOPHENMETHOCARBAMOL:Gastric disorder:0.23), (ACETYLCARNITINE HYDROCHLORIDE:Gastric disorder:0.11), (AGALSIDASE BETA:Gastric disorder:1.29), (ALBIGLUTIDE:Gastric disorder:0.36), (ALCOHOL:Gastric disorder:0.9), (ALDESLEUKIN:Gastric disorder:0.12), (ALISKIREN HEMIFUMARATEHYDROCHLOROTHIAZIDE:Gastric disorder:0.17), (AMINO ACIDSDEXTROSEELECTROLYTES NOS:Gastric disorder:0.88), (AMINOPHYLLINE:Gastric disorder:0.05), (AMINOSALICYLIC ACID:Gastric disorder:0.12), (AMISULPRIDE:Gastric disorder:1.26), (AMLODIPINEPERINDOPRIL:Gastric disorder:0.34), (AMMONIUM CHLORIDEDEXTROMETHORPHANSODIUM CITRATE:Gastric disorder:0.05), (ANAKINRA:Gastric disorder:0.59), (ANTIHISTAMINES NOS:Gastric disorder:0.83), (ARIPIPRAZOLE LAUROXIL:Gastric disorder:0.5), (ASCORBIC ACIDBIOTINCALCIUM PANTOTHENATECOBALAMINFOLIC ACIDNIACINAMIDEPYRIDOXINE HYDROCHLORIDERIBOFLAVINTHIAMINE MONONITRATE:Gastric disorder:0.35), (ASIAN GINSENG:Gastric disorder:0.12), (ASPIRIN DL-LYSINE:Gastric disorder:0.01), (ATROPINE SULFATE:Gastric disorder:0.24), (BETAMETHASONE DIPROPIONATECALCIPOTRIENE:Gastric disorder:0.52), (BETAMETHASONE DIPROPIONATECALCIPOTRIENECALCIPOTRIENE HYDRATE:Gastric disorder:1.08), (BETAMETHASONE VALERATE:Gastric disorder:0.41), (BICARBONATE ION:Gastric disorder:0.09), (BILASTINE:Gastric disorder:0.46), (BOTULINUM TOXIN TYPE A:Gastric disorder:0.11), (BROMFENAC SODIUM:Gastric disorder:0.13), (CABOZANTINIB S-MALATE:Gastric disorder:0.35), (CALCIUM CARBONATEERGOCALCIFEROLRETINOL:Gastric disorder:0.53), (CALCIUMMAGNESIUMZINC:Gastric disorder:1.19), (CARBOMER:Gastric disorder:0), (CARTEOLOL HYDROCHLORIDE:Gastric disorder:0.08), (CARVEDILOL PHOSPHATE:Gastric disorder:2.47), (CASANTHRANOLDOCUSATE SODIUM:Gastric disorder:0.54), (CASPOFUNGIN:Gastric disorder:0.46), (CEFAZOLIN:Gastric disorder:0.7), (CEFEPIME HYDROCHLORIDE:Gastric disorder:0.76), (CELIPROLOL:Gastric disorder:0.09), (CERIVASTATIN SODIUM:Gastric disorder:0.58), (CERTOPARIN SODIUM:Gastric disorder:0.01), (CHLORAMBUCIL:Gastric disorder:0.61), (CHLORPHENIRAMINEHYDROCODONE:Gastric disorder:0.67), (CHONDROITIN SULFATE (CHICKEN):Gastric disorder:0.12), (CICLOPIROX:Gastric disorder:0.1), (CLOFAZIMINE:Gastric disorder:0.03), (CODEINE PHOSPHATEGUAIFENESIN:Gastric disorder:0.63), (COLISTIN:Gastric disorder:0.01), (CORTICOTROPIN:Gastric disorder:1.1), (CURCUMIN:Gastric disorder:0.2), (DANTROLENE SODIUM:Gastric disorder:0.4), (DAPAGLIFLOZIN PROPANEDIOLMETFORMIN HYDROCHLORIDE:Gastric disorder:0.25), (DAPSONE:Gastric disorder:0.92), (DEFERIPRONE:Gastric disorder:0.07), (DEGARELIX:Gastric disorder:0.12), (DEXTROAMPHETAMINE:Gastric disorder:0.45), (DEXTROAMPHETAMINE SULFATE:Gastric disorder:1.55), (DIFLUNISAL:Gastric disorder:0.37), (DIFLUPREDNATE:Gastric disorder:0.22), (DIMETHINDENE:Gastric disorder:0.08), (DIPHENHYDRAMINE HYDROCHLORIDELIDOCAINE HYDROCHLORIDENYSTATIN:Gastric disorder:0.17), (DIPYRIDAMOLE:Gastric disorder:1.37), (DOCUSATE SODIUMSENNOSIDES:Gastric disorder:0.25), (DOLASETRON MESYLATE:Gastric disorder:0.61), (DROXIDOPA:Gastric disorder:0.29), (DUTASTERIDETAMSULOSIN HYDROCHLORIDE:Gastric disorder:1.06), (ECONAZOLE:Gastric disorder:1.23), (ELBASVIRGRAZOPREVIR:Gastric disorder:0.32), (ELTROMBOPAG OLAMINE:Gastric disorder:0.27), (EMPAGLIFLOZINLINAGLIPTIN:Gastric disorder:1.24), (ENALAPRIL MALEATELERCANIDIPINE HYDROCHLORIDE:Gastric disorder:0.03), (EPINASTINE HYDROCHLORIDE:Gastric disorder:0.01), (EPIRUBICIN:Gastric disorder:0.57), (ERIBULIN:Gastric disorder:0.59), (ERTAPENEM SODIUM:Gastric disorder:0.18), (ETHACRYNIC ACID:Gastric disorder:0.45), (ETHINYL ESTRADIOLGESTODENE:Gastric disorder:0.2), (ETHINYL ESTRADIOLNORGESTREL:Gastric disorder:0.59), (ETRETINATE:Gastric disorder:0.03), (EVOLOCUMAB:Gastric disorder:2.31), (FERROUS SULFATEGLYCINE:Gastric disorder:0.71), (FLURAZEPAMFLURAZEPAM HYDROCHLORIDE:Gastric disorder:0.32), (FLUVOXAMINE MALEATE:Gastric disorder:1.44), (FORMOTEROL FUMARATE DIHYDRATE:Gastric disorder:0.01), (FORMOTEROL FUMARATEMOMETASONE FUROATE:Gastric disorder:1.08), (FOSFOMYCIN:Gastric disorder:0.21), (FROVATRIPTAN SUCCINATE:Gastric disorder:0.15), (FUSIDIC ACID:Gastric disorder:1.38), (GEFITINIB:Gastric disorder:0.41), (GLYCOPYRROLATEINDACATEROL MALEATE:Gastric disorder:0.77), (HEPARIN CALCIUM:Gastric disorder:0.82), (HERBALSTURMERIC:Gastric disorder:1.65), (HORSE CHESTNUT:Gastric disorder:0.13), (HYALURONIC ACID:Gastric disorder:0.04), (HYOSCYAMINE SULFATEMETHENAMINEMETHYLENE BLUEPHENYL SALICYLATESODIUM PHOSPHATE, MONOBASIC, MONOHYDRATE:Gastric disorder:0.25), (IBRITUMOMAB TIUXETAN:Gastric disorder:0.17), (IMIGLUCERASE:Gastric disorder:0.49), (IMIQUIMOD:Gastric disorder:0.12), (INDAPAMIDEPERINDOPRIL:Gastric disorder:2.47), (INSULIN PORKINSULIN PURIFIED PORK:Gastric disorder:0.2), (INTERFERON ALFA-2A:Gastric disorder:1.37), (INTERFERON ALFA-2B:Gastric disorder:0.55), (INTERFERON GAMMA-1B:Gastric disorder:0.33), (ITRACONAZOLE:Gastric disorder:1.31), (IVERMECTIN:Gastric disorder:0.35), (LANREOTIDE:Gastric disorder:0.46), (LEVOLEUCOVORIN CALCIUM:Gastric disorder:0.76), (LEVOMILNACIPRAN HYDROCHLORIDE:Gastric disorder:0.22), (LOMITAPIDE MESYLATE:Gastric disorder:1.07), (MAGNESIUM GLUCONATE:Gastric disorder:0.61), (MECLIZINE MONOHYDROCHLORIDENIACIN:Gastric disorder:0.09), (MEPOLIZUMAB:Gastric disorder:0.73), (MESNA:Gastric disorder:0.35), (METFORMIN HYDROCHLORIDEROSIGLITAZONE MALEATE:Gastric disorder:0.03), (METFORMIN PAMOATE:Gastric disorder:0.12), (METHENAMINE MANDELATE:Gastric disorder:0.33), (METHENOLONE ENANTHATE:Gastric disorder:0.11), (METRELEPTIN:Gastric disorder:0.4), (MINOCYCLINE HYDROCHLORIDE:Gastric disorder:1.17), (MOEXIPRIL HYDROCHLORIDE:Gastric disorder:0.29), (NISOLDIPINE:Gastric disorder:0.12), (NOREPINEPHRINE BITARTRATE:Gastric disorder:0.08), (OLAPARIB:Gastric disorder:0.47), (OLOPATADINE:Gastric disorder:0.47), (OXYBUTYNINOXYBUTYNIN CHLORIDE:Gastric disorder:0.67), (PAMIDRONATE DISODIUM:Gastric disorder:1.31), (PASIREOTIDE:Gastric disorder:1.06), (PATIROMER:Gastric disorder:0.91), (PEGINTERFERON BETA-1A:Gastric disorder:1.42), (PENICILLIN G:Gastric disorder:0.76), (PENTOSTATIN:Gastric disorder:0.22), (PNEUMOCOCCAL VACCINE, POLYVALENT 23:Gastric disorder:0.64), (POLYETHYLENE GLYCOL 3350\POTASSIUM CHLORIDESODIUM BICARBONATESODIUM CHLORIDESODIUM SULFATE ANHYDROUS:Gastric disorder:0.62), (PRAMLINTIDE ACETATE:Gastric disorder:0.13), (PRANLUKAST:Gastric disorder:0.16), (PRAZEPAM:Gastric disorder:0.14), (QUINACRINE:Gastric disorder:0.6), (QUINAPRIL HYDROCHLORIDE:Gastric disorder:0.26), (RADIATION THERAPY:Gastric disorder:1.03), (RILMENIDINE PHOSPHATE:Gastric disorder:0.33), (ROFECOXIB:Gastric disorder:0.01), (RUCAPARIB CAMSYLATE:Gastric disorder:0.08), (SERRAPEPTASE:Gastric disorder:0.01), (SIPULEUCEL-T:Gastric disorder:0.4), (SODIUM PICOSULFATE:Gastric disorder:0.34), (SOFOSBUVIRVELPATASVIR:Gastric disorder:0.63), (TEDUGLUTIDEWATER:Gastric disorder:0.54), (TELOTRISTAT ETHYL:Gastric disorder:0.59), (TEMSIROLIMUS:Gastric disorder:0.08), (TERBUTALINE:Gastric disorder:0.2), (THALIDOMIDE:Gastric disorder:2.07), (TIGECYCLINE:Gastric disorder:0.36), (TOFACITINIB:Gastric disorder:0.14), (TRIMIPRAMINE MALEATE:Gastric disorder:1.02), (TRIPTORELIN PAMOATE:Gastric disorder:0.69), (UBIQUINOL:Gastric disorder:0.12), (UMECLIDINIUM BROMIDE:Gastric disorder:1.03), (UMECLIDINIUM BROMIDEVILANTEROL TRIFENATATE:Gastric disorder:1.34), (URAPIDIL:Gastric disorder:0.58), (VALDECOXIB:Gastric disorder:0.29), (VINORELBINE TARTRATE:Gastric disorder:0.58), (VISMODEGIB:Gastric disorder:0.08), (ACARBOSE:Gastric pH decreased:0.15), (ACETAMINOPHENOXYCODONEOXYCODONE HYDROCHLORIDE:Gastric pH decreased:0.18),(ARMODAFINIL:Gastric pH decreased:0.89), (BUPRENORPHINE HYDROCHLORIDENALOXONE HYDROCHLORIDE:Gastric pH decreased:0.27), (CALCIUM CITRATE:Gastric pH decreased:0.58), (CANAGLIFLOZINMETFORMIN HYDROCHLORIDE:Gastric pH decreased:0.06), (CANDESARTAN CILEXETILHYDROCHLOROTHIAZIDE:Gastric pH decreased:0.88), (CARFILZOMIB:Gastric pH decreased:0.37), (CERTOLIZUMAB PEGOL:Gastric pH decreased:1.35), (CLOZAPINE:Gastric pH decreased:0.52), (CORTISONEHYDROCORTISONE:Gastric pH decreased:0.29), (CYANOCOBALAMINDEXPANTHENOLNIACINAMIDEPYRIDOXINE HYDROCHLORIDERIBOFLAVIN 5′-PHOSPHATE SODIUMTHIAMINE HYDROCHLORIDEVITAMIN B COMPLEX:Gastric pH decreased:0.4), (DACLIZUMAB:Gastric pH decreased:0.09), (DAPAGLIFLOZIN PROPANEDIOL:Gastric pH decreased:0.29), (DASATINIB:Gastric pH decreased:0.5), (DESVENLAFAXINE SUCCINATE:Gastric pH decreased:0.02), (DIMENHYDRINATE:Gastric pH decreased:0.2), (DOFETILIDE:Gastric pH decreased:0.44), (DOXYCYCLINE:Gastric pH decreased:0.79), (EDOXABAN TOSYLATE:Gastric pH decreased:0.46), (ELETRIPTAN HYDROBROMIDE:Gastric pH decreased:0.36), (EPINEPHRINE:Gastric pH decreased:0.5), (EPLERENONE:Gastric pH decreased:0.19), (ESOMEPRAZOLE MAGNESIUMNAPROXEN:Gastric pH decreased:0), (ESTROGENS, CONJUGATEDMEDROXYPROGESTERONE ACETATE:Gastric pH decreased:0.16), (EVEROLIMUS:Gastric pH decreased:0.61), (EVOLOCUMAB:Gastric pH decreased:0.58), (FESOTERODINE FUMARATE:Gastric pH decreased:0.39), (FLECAINIDE:Gastric pH decreased:0.7), (HYDROXYCHLOROQUINE DIPHOSPHATE:Gastric pH decreased:0.09), (HYDROXYZINE PAMOATE:Gastric pH decreased:0.96), (ILOPROST:Gastric pH decreased:0.31), (INDAPAMIDE:Gastric pH decreased:0.29), (INDAPAMIDEPERINDOPRIL:Gastric pH decreased:0.26), (INSULIN GLULISINE:Gastric pH decreased:0.19), (LABETALOLLABETALOL HYDROCHLORIDE:Gastric pH decreased:0.73), (LEFLUNOMIDE:Gastric pH decreased:0.12), (LERCANIDIPINE:Gastric pH decreased:1.15), (LEVALBUTEROL HYDROCHLORIDE:Gastric pH decreased:0.37), (METHIMAZOLE:Gastric pH decreased:0.26), (METHYLPHENIDATE HYDROCHLORIDE:Gastric pH decreased:0.48), (METHYLPREDNISOLONE ACETATE:Gastric pH decreased:0.24), (METHYLPREDNISOLONE SODIUM SUCCINATE:Gastric pH decreased:1.09), (MIDODRINE:Gastric pH decreased:0.18), (NALOXONE HYDROCHLORIDEOXYCODONE HYDROCHLORIDE:Gastric pH decreased:0.53), (NEBIVOLOL:Gastric pH decreased:0.56), (OXCARBAZEPINE:Gastric pH decreased:0.92), (PEGFILGRASTIM:Gastric pH decreased:0.11), (PEGINTERFERON BETA-1A:Gastric pH decreased:0.05), (PENICILLIN:Gastric pH decreased:0.54), (PONATINIB:Gastric pH decreased:0.33), (PRIMIDONE:Gastric pH decreased:0.63), (PROGESTERONE:Gastric pH decreased:0.01), (SACUBITRILVALSARTAN:Gastric pH decreased:0.93), (SCOPOLAMINE:Gastric pH decreased:0.36), (SECUKINUMAB:Gastric pH decreased:0.02), (TELMISARTAN:Gastric pH decreased:0.92), (TERIFLUNOMIDE:Gastric pH decreased:0.45), (TETRACYCLINE:Gastric pH decreased:0.63), (TIOTROPIUM:Gastric pH decreased:0.14), (UMECLIDINIUM BROMIDEVILANTEROL TRIFENATATE:Gastric pH decreased:0.27), (.ALPHA.-TOCOPHEROLRETINOL:Gastric ulcer:0.77), (.ALPHA.1-PROTEINASE INHIBITOR HUMAN:Gastric ulcer:0.15), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Gastric ulcer:0.02), (ACETAMINOPHENCAFFEINE:Gastric ulcer:0.26), (ACETAMINOPHENOXYCODONEOXYCODONE HYDROCHLORIDE:Gastric ulcer:1.16), (ACETAMINOPHENPSEUDOEPHEDRINE HYDROCHLORIDE:Gastric ulcer:0.01), (ACITRETIN:Gastric ulcer:0.44), (ADENINE:Gastric ulcer:1.37), (ALBIGLUTIDE:Gastric ulcer:0.1), (ALBUMIN BOVINETANNIC ACID:Gastric ulcer:1), (ALBUTEROLIPRATROPIUM:Gastric ulcer:0.25), (ALDESLEUKIN:Gastric ulcer:0.27), (ALECTINIB:Gastric ulcer:0.1), (ALEMTUZUMAB:Gastric ulcer:0.67), (ALUMINUM SILICATE:Gastric ulcer:0.91), (AMINOSALICYLIC ACID:Gastric ulcer:0.14), (AMLODIPINEPERINDOPRIL:Gastric ulcer:0.46), (AMMONIUM GLYCYRRHIZATECYSTEINE HYDROCHLORIDEGLYCINE:Gastric ulcer:0.28), (ANASTROZOLE:Gastric ulcer:2.52), (ATRACURIUM:Gastric ulcer:0.22), (ATROPINEDIPHENOXYLATE:Gastric ulcer:0.01), (BECLOMETHASONE:Gastric ulcer:0.25), (BECLOMETHASONEFORMOTEROL:Gastric ulcer:0.02), (BENZTROPINE MESYLATE:Gastric ulcer:0.07), (BERAPROST:Gastric ulcer:0.55), (BETAMETHASONE BUTYRATE PROPIONATE:Gastric ulcer:1.21), (BETAMETHASONE DIPROPIONATECALCIPOTRIENE:Gastric ulcer:0.03), (BETAMETHASONE DIPROPIONATECALCIPOTRIENECALCIPOTRIENE HYDRATE:Gastric ulcer:0.36), (BETAMETHASONE DIPROPIONATECLOTRIMAZOLE:Gastric ulcer:0.11), (BETAMETHASONE SODIUM PHOSPHATE:Gastric ulcer:0.46), (BETAMETHASONE SODIUM PHOSPHATENEOMYCIN SULFATE:Gastric ulcer:0.56), (BETAMETHASONECALCIPOTRIENE:Gastric ulcer:0.57), (BETAMETHASONEDEXCHLORPHENIRAMINE MALEATE:Gastric ulcer:0.28), (BETHANECHOL CHLORIDE:Gastric ulcer:0.33), (BICARBONATE IONPOLYETHYLENE GLYCOLSPOTASSIUM CATIONSODIUM CHLORIDESODIUM SULFATE ANHYDROUS:Gastric ulcer:0.24), (BLINATUMOMAB:Gastric ulcer:0.51), (BLONANSERIN:Gastric ulcer:0.03), (BUFORMIN:Gastric ulcer:0.19), (BUPRENORPHINE HYDROCHLORIDENALOXONE HYDROCHLORIDE:Gastric ulcer:1.13), (CALCITONIN SALMON:Gastric ulcer:0.96), (CALCIUM CARBONATEVITAMIN D:Gastric ulcer:0.56), (CALCIUM CHLORIDE ANHYDROUSPOTASSIUM CHLORIDESODIUM CHLORIDESODIUM LACTATE:Gastric ulcer:0.75), (CAMPHORMENTHOLMETHYL SALICYLATE:Gastric ulcer:0.81), (CANAGLIFLOZINMETFORMIN HYDROCHLORIDE:Gastric ulcer:1.16), (CANNABIS SATIVA SUBSP. INDICA TOP:Gastric ulcer:0.1), (CANRENOATE POTASSIUM:Gastric ulcer:0), (CARBOXYMETHYLCELLULOSE SODIUM:Gastric ulcer:0), (CASANTHRANOLDOCUSATE SODIUM:Gastric ulcer:0.35), (CASPOFUNGIN:Gastric ulcer:0.34), (CEFOTAXIME SODIUM:Gastric ulcer:0.92), (CEFOTAXIMECEFOTAXIME SODIUM:Gastric ulcer:0.21), (CEFPIROME SULFATE:Gastric ulcer:0.03), (CEFUROXIME AXETIL:Gastric ulcer:0.06), (CEPHARANTHINE:Gastric ulcer:1.21), (CETIRIZINE HYDROCHLORIDEPSEUDOEPHEDRINE HYDROCHLORIDE:Gastric ulcer:0.09), (CHLORAMBUCIL:Gastric ulcer:0.7), (CHLORPHENIRAMINE MALEATDIHYDROCODEINE PHOSPHATEMETHYLEPHEDRINE HYDROCHLORIDE, DL-:Gastric ulcer:0.48), (CHLORPHENIRAMINEHYDROCODONE:Gastric ulcer:0.28), (CHLORPROMAZINEPROMETHAZINE:Gastric ulcer:0.11), (CHONDROITIN SULFATE (BOVINE)GLUCOSAMINE HYDROCHLORIDE:Gastric ulcer:0.31), (CICLESONIDE:Gastric ulcer:0.58), (CLENBUTEROL HYDROCHLORIDE:Gastric ulcer:0.91), (CLINDAMYCIN HYDROCHLORIDE:Gastric ulcer:0.03), (CLOMIPRAMINE HYDROCHLORIDE:Gastric ulcer:0.44), (COD LIVER OIL:Gastric ulcer:0.14), (CORTICOTROPIN:Gastric ulcer:0.55), (CRIZOTINIB:Gastric ulcer:1.2), (CUPRIC SULFATEFERRIC CHLORIDEMANGANESE CHLORIDEPOTASSIUM IODIDEZINC SULFATE:Gastric ulcer:0), (CYPROHEPTADINE HYDROCHLORIDE:Gastric ulcer:0.01), (DACLIZUMAB:Gastric ulcer:1.21), (DALFAMPRIDINE:Gastric ulcer:0.91), (DANAZOL:Gastric ulcer:1.36), (DAPAGLIFLOZIN PROPANEDIOL:Gastric ulcer:0.74), (DEFERIPRONE:Gastric ulcer:0.09), (DEFLAZACORT:Gastric ulcer:1.66), (DEXTROSEPOTASSIUM CHLORIDESODIUM CHLORIDESODIUM LACTATE:Gastric ulcer:0.23), (DIMEMORFAN PHOSPHATE:Gastric ulcer:0.56), (DIQUAFOSOL TETRASODIUM:Gastric ulcer:0.46), (DOFETILIDE:Gastric ulcer:0.86), (DOLASETRON MESYLATE:Gastric ulcer:0.15), (DORZOLAMIDE:Gastric ulcer:0.22), (DOXEPIN:Gastric ulcer:0.82), (DOXEPIN HYDROCHLORIDE:Gastric ulcer:0.6), (DRONEDARONE:Gastric ulcer:0.46), (DUTASTERIDETAMSULOSIN HYDROCHLORIDE:Gastric ulcer:0.56), (ELBASVIRGRAZOPREVIR:Gastric ulcer:0.05), (EMPAGLIFLOZINLINAGLIPTIN:Gastric ulcer:0.46), (EPHEDRA SINICA STEMHERBALS:Gastric ulcer:0.16), (EPRAZINONE:Gastric ulcer:0.18), (ERIBULIN:Gastric ulcer:0.44), (ERYTHROMYCIN LACTOBIONATE:Gastric ulcer:0.12), (ESTROGENS, CONJUGATEDMEDROXYPROGESTERONE ACETATE:Gastric ulcer:0.65), (ETHYL ICOSAPENTATEICOSAPENT:Gastric ulcer:1.51), (EZETIMIBESIMVASTATIN:Gastric ulcer:1.25), (FAMCICLOVIR:Gastric ulcer:0.04), (FAROPENEM SODIUM:Gastric ulcer:1.12), (FENOTEROL HYDROBROMIDE:Gastric ulcer:0.19), (FERRIC HYDROXIDE:Gastric ulcer:0.55), (FERROUS SULFATEFOLIC ACID:Gastric ulcer:0.26), (FERROUS SULFATEGLYCINE:Gastric ulcer:0.33), (FISH OIL\OMEGA-3 FATTY ACIDS:Gastric ulcer:0.21), (FLOMOXEF SODIUM:Gastric ulcer:0.03), (FLURAZEPAMFLURAZEPAM HYDROCHLORIDE:Gastric ulcer:0.02), (FOLATE SODIUM:Gastric ulcer:0.43), (FONDAPARINUX:Gastric ulcer:0.46), (FORMOTEROL:Gastric ulcer:0.73), (FORMOTEROL FUMARATEMOMETASONE FUROATE:Gastric ulcer:0.1), (FOSFOMYCIN:Gastric ulcer:0.02), (FUDOSTEINE:Gastric ulcer:0.41), (FURSULTIAMINE:Gastric ulcer:1.12), (FUSIDIC ACID:Gastric ulcer:1.19), (GLUCAGON:Gastric ulcer:0.15), (GLYCOPYRROLATEINDACATEROL MALEATE:Gastric ulcer:0.17), (GRANULOCYTE COLONY-STIMULATING FACTOR NOS:Gastric ulcer:0.62), (GUANFACINE:Gastric ulcer:0.29), (GUANFACINE HYDROCHLORIDE:Gastric ulcer:0.51), (HERBALSSAW PALMETTO:Gastric ulcer:0.34), (HERBALSTURMERIC:Gastric ulcer:0.52), (HUMAN PLASMA:Gastric ulcer:0.13), (HYDROCHLOROTHIAZIDEQUINAPRIL HYDROCHLORIDE:Gastric ulcer:0.02), (HYDROCODONE BITARTRATEIBUPROFEN:Gastric ulcer:0.13), (HYDROXYCHLOROQUINE DIPHOSPHATE:Gastric ulcer:0.61), (IBRITUMOMAB TIUXETAN:Gastric ulcer:0.67), (IDARUCIZUMAB:Gastric ulcer:0.55), (IMIGLUCERASE:Gastric ulcer:0.46), (INDAPAMIDEPERINDOPRIL:Gastric ulcer:1.5), (INTERFERON:Gastric ulcer:1.08), (INTERFERON ALFA-2A:Gastric ulcer:0.83), (INTERFERON ALFA-2B:Gastric ulcer:1.03), (INTERFERON ALFA-Nl:Gastric ulcer:0.04), (IODINE:Gastric ulcer:0.13), (ISOFLURANE:Gastric ulcer:0.45), (LABETALOL HYDROCHLORIDE:Gastric ulcer:0.85), (LACTASE:Gastric ulcer:0.16), (LAPATINIB DITOSYLATE:Gastric ulcer:0.8), (LEVALBUTEROL:Gastric ulcer:0.05), (LEVALBUTEROL HYDROCHLORIDE:Gastric ulcer:1.39), (LICORICE ROOT EXTRACTRHUBARB:Gastric ulcer:0.31), (LINSIDOMINE:Gastric ulcer:0.02), (LIOTHYRONINE:Gastric ulcer:0.19), (LIOTHYRONINE SODIUM:Gastric ulcer:0.41), (LISDEXAMFETAMINE DIMESYLATE:Gastric ulcer:1.15), (LOBUCAVIR:Gastric ulcer:0.23), (LORNOXICAM:Gastric ulcer:0.12), (LUTEIN:Gastric ulcer:0.01), (LYSOZYME HYDROCHLORIDE:Gastric ulcer:1.21), (MAGNESIUM ASPARTATEPOTASSIUM ASPARTATE:Gastric ulcer:0), (MANIDIPINE HYDROCHLORIDE:Gastric ulcer:0), (MEPENZOLATE BROMIDE:Gastric ulcer:0.35), (MEPOLIZUMAB:Gastric ulcer:0.14), (METFORMIN HYDROCHLORIDESAXAGLIPTIN HYDROCHLORIDE:Gastric ulcer:0.04), (METFORMIN PAMOATE:Gastric ulcer:0.1), (METHENAMINE HIPPURATE:Gastric ulcer:0.98), (METHENOLONE ACETATE:Gastric ulcer:0.15), (MICAFUNGIN:Gastric ulcer:0.59), (MICONAZOLE NITRATE:Gastric ulcer:0.58), (MIRIPLATIN:Gastric ulcer:0.19), (MITIGLINIDE CALCIUM HYDRATEVOGLIBOSE:Gastric ulcer:0.79), (MOEXIPRIL HYDROCHLORIDE:Gastric ulcer:0.55), (NALFURAFINE HYDROCHLORIDE:Gastric ulcer:0.02), (NALOXONE HYDROCHLORIDETILIDINE HYDROCHLORIDE:Gastric ulcer:0.8), (NEOSTIGMINE:Gastric ulcer:0.67), (NICERGOLINE:Gastric ulcer:0.03), (NILVADIPINE:Gastric ulcer:0.03), (NIRAPARIB:Gastric ulcer:0.75), (NORETHINDRONE:Gastric ulcer:0.61), (OK-432:Gastric ulcer:0.11), (OLMESARTAN:Gastric ulcer:0.42), (OSELTAMIVIR:Gastric ulcer:0.21), (PALBOCICLIB:Gastric ulcer:0.38), (PAMIDRONATE DISODIUM:Gastric ulcer:0.47), (PARICALCITOL:Gastric ulcer:0.73), (PATIROMER:Gastric ulcer:0.37), (PAZUFLOXACIN MESILATE:Gastric ulcer:0.21), (PEGINTERFERON BETA-1A:Gastric ulcer:1.01), (PENICILLIN G:Gastric ulcer:0.35), (PENTOSTATIN:Gastric ulcer:0.04), (PIROXICAM:Gastric ulcer:0.77), (POLYSTYRENE SULFONIC ACID:Gastric ulcer:0.89), (POTASSIUM CITRATE:Gastric ulcer:0.01), (POTASSIUM IODIDE:Gastric ulcer:0.42), (PREDNISOLONE SODIUM PHOSPHATE:Gastric ulcer:0.37), (PRISTINAMYCIN:Gastric ulcer:0.25), (PROBUCOL:Gastric ulcer:0.48), (QUINACRINE:Gastric ulcer:0.21), (QUINAPRIL:Gastric ulcer:0.26), (RADIATION THERAPY:Gastric ulcer:0.95), (RIVASTIGMINE:Gastric ulcer:0.94), (RIZATRIPTAN BENZOATE:Gastric ulcer:1.19), (SACCHAROMYCES CEREVISIAE:Gastric ulcer:0.23), (SCOPOLAMINE HYDROBROMIDE:Gastric ulcer:0.21), (SERRAPEPTASE:Gastric ulcer:0.1), (SODIUM CITRATE:Gastric ulcer:0.05), (SODIUM FLUORIDE:Gastric ulcer:0.55), (SODIUM GUALENATE MONOHYDRATE:Gastric ulcer:0.57), (SODIUM PHOSPHATE, DIBASICSODIUM PHOSPHATE, MONOBASIC:Gastric ulcer:0.38), (SOFALCONE:Gastric ulcer:0.15), (SORBITOL:Gastric ulcer:0.1), (SOTALOL HYDROCHLORIDE:Gastric ulcer:0.23), (STREPTOMYCIN SULFATE:Gastric ulcer:0.25), (SUCCINYLCHOLINE CHLORIDE:Gastric ulcer:0.43), (SUFENTANIL:Gastric ulcer:0.15), (SULINDAC:Gastric ulcer:0.05), (SUPLATAST TOSILATE:Gastric ulcer:0.98), (TAMOXIFEN CITRATE:Gastric ulcer:0.14), (TEDUGLUTIDEWATER:Gastric ulcer:0.26), (TEGAFURURACIL:Gastric ulcer:0.23), (TERIFLUNOMIDE:Gastric ulcer:0.48), (TESTOSTERONE UNDECANOATE:Gastric ulcer:0.36), (TETRACYCLINE:Gastric ulcer:0.91), (THIOPENTAL:Gastric ulcer:0.79), (THIOPENTAL SODIUM:Gastric ulcer:0.05), (THROMBOMODULIN ALFA:Gastric ulcer:0.87), (TIARAMIDE:Gastric ulcer:0.1), (TIOTROPIUM:Gastric ulcer:1.27), (TIPEPIDINE HIBENZATE:Gastric ulcer:1.06), (TOBRAMYCIN:Gastric ulcer:0.11), (TOCOPHEROL:Gastric ulcer:0.57), (TOPIROXOSTAT:Gastric ulcer:0.01), (TOSUFLOXACIN TOSYLATE:Gastric ulcer:0.63), (TRANDOLAPRIL:Gastric ulcer:0.4), (TRAVOPROST:Gastric ulcer:0.05), (TROSPIUM CHLORIDE:Gastric ulcer:0.72), (UMECLIDINIUM BROMIDE:Gastric ulcer:0.42), (VINDESINE SULFATE:Gastric ulcer:0.02), (ZAFIRLUKAST:Gastric ulcer:0.12), (ZILEUTON:Gastric ulcer:0.03), (ZOTEPINE:Gastric ulcer:0.17), (.ALPHA.-TOCOPHEROLRETINOL:Gastritis:1.12), (ACEBUTOLOL HYDROCHLORIDE:Gastritis:0.64), (ACETAMINOPHENAPRONALIDECAFFEINEPROP YPHENAZONE:Gastritis:1.07), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Gastritis:0.42), (ACETAMINOPHENOXYCODONEOXYCODONE HYDROCHLORIDE:Gastritis:1.58), (ACETAMINOPHENPROPOXYPHENE NAPSYLATE:Gastritis:0.07), (ACETAMINOPHENPSEUDOEPHEDRINE HYDROCHLORIDE:Gastritis:0.37), (ACOTIAMIDE HYDROCHLORIDE:Gastritis:0.17), (ADENINE:Gastritis:1.99), (ADUCANUMAB:Gastritis:0.08), (ALBUTEROLIPRATROPIUM:Gastritis:0.54), (ALDESLEUKIN:Gastritis:0.23), (ALISKIR EN HEMIFUMARATE:Gastritis:0.8), (ALUMINUM SILICATE:Gastritis:1.22), (AMINO ACIDSCARBOHYDRATESELECTROLYTES NOSVITAMINS:Gastritis:1.14), (AMINO ACIDSDEXTROSEELECTROLYTES NOS:Gastritis:0.91), (AMINO ACIDSVITAMINS:Gastritis:0.02), (AMLODIPINE BESYLATEAZILSARTAN:Gastritis:0), (AMLODIPINE BESYLATEHYDROCHLOROTHIAZIDEOLMESARTAN MEDOXOMIL:Gastritis:0.51), (AMMONIUM LACTATE:Gastritis:0.45), (AMPICILLIN SODIUM:Gastritis:0.38), (ANAGRELIDE HYDROCHLORIDE:Gastritis:0.82), (ANAKINRA:Gastritis:0.09), (ANTIHISTAMINES NOS:Gastritis:0.39), (ARIPIPRAZOLE LAUROXIL:Gastritis:0.1), (AROTINOLOL:Gastritis:0.27), (ASPIRINLANSOPRAZOLE:Gastritis: 1.4), (ATORVASTATIN CALCIUMEZETIMIBE:Gastritis:0.28), (ATROPINE SULFATE:Gastritis:0.52), (AZITHROMYCIN OR METHOTREXATE SODIUM:Gastritis:0.25), (BECLOMETHASONE DIPROPIONATEFORMOTEROL:Gastritis:0.13), (BENPROPERINE PHOSPHATE:Gastritis:0.67), (BERAPROST:Gastritis:0.44), (BETAMETHASONE DIPROPIONATECALCIPOTRIENE:Gastritis:0.62), (BETAMETHASONE DIPROPIONATECALCIPOTRIENECALCIPOTRIENE HYDRATE:Gastritis:0.75), (BETAMETHASONE DIPROPIONATECLOTRIMAZOLE:Gastritis:0.66), (BETAMETHASONE SODIUM PHOSPHATE:Gastritis:0.74), (BETAMETHASONE SODIUM PHOSPHATENEOMYCIN SULFATE:Gastritis:0.82), (BETAMIPRONPANIPENEM:Gastritis:0.34), (BIFIDOBACTERIU M LONGUM:Gastritis:0.56), (BIMATOPROSTTIMOLOL:Gastritis:0.17), (BROMHEXINE:Gastritis: 0.26), (BUCOLOME:Gastritis:0.37), (BUFORMIN:Gastritis:0.55), (BUTYLSCOPOLAMINE: Gastritis:0.08), (CABOZANTINIB:Gastritis:0), (CALCITONIN:Gastritis:1.28), (CALCITONIN SALMON:Gastritis:1.49), (CALCIUMMAGNESIUM:Gastritis:0.71), (CALCIUMMAGNESIU MZINC:Gastritis:0.77), (CAMPHORMENTHOLMETHYL SALICYLATE:Gastritis:1.17), (CAPSAICIN:Gastritis:0.25), (CARMUSTINE:Gastritis:0.06), (C ARVEDILOL PHOSPHATE:Gastritis:2.16), (CEFCAPENE PIVOXIL:Gastritis:0.1), (CEFOTAXIMECEFOTAXIME SODIUM:Gastritis:0.19), (CEFPIROME SULFATE:Gastritis:0.06), (CEFPODOXIME PROXETIL:Gastritis:1.05), (CEFTERAM PIVOXIL:Gastritis:0.38), (CETIRIZINE HYDROCHLORIDEPSEUDOEPHEDRINE HYDROCHLORIDE:Gastritis:0.16), (CEVIMELINE HYDROCHLORIDE:Gastritis:0.14), (CHLORPHENIRAMINE MALEATDIHYDROCODEINE PHOSPHATEMETHYLEPHEDRINE HYDROCHLORIDE, DL-:Gastritis:0.85), (CHLORPHENIRAMINEHYDROCODONE:Gastritis:0.55), (CHONDROI TIN SULFATE (CHICKEN):Gastritis:0.05), (CHONDROITIN SULFATE AGLUCOSAMINE:Gastritis:0.06), (CLENBUTEROL HYDROCHLORIDE:Gastritis:1.21), (CLOPERASTINE:Gastritis:1.09), (COBICISTATELVITE GRAVIREMTRICITABINETENOFOVIR ALAFENAMIDE FUMARATE:Gastritis:0.18), (COLESEVELAM HYDROCHLORIDE:Gastritis:0.81), (COLESTILAN CHLORIDE:Gastritis:0.46), (COLISTIMETHATE SODIUM:Gastritis:0.11), (CONVALLARIA MAJALISCRATAEGUS LAEVIGATA LEAFPROXYPHYLLINE:Gastritis:0.01), (CUPRIC SULFATEFERRIC CHLORIDEMANGANESE CHLORIDEPOTASSIUM IODIDEZINC SULFATE:Gastritis:0.06), (CYAMEMAZINE:Gastritis:0.47), (CYANAMIDE:Gastritis:0.3), (DA NAPAROID SODIUM:Gastritis:0.13), (DANTROLENE SODIUM:Gastritis:0.62), (DAPSONE:Gastritis:1.51), (DESMOPRESSIN:Gastritis:0.5), (DEXTR OAMPHETAMINE SULFATE:Gastritis:0.46), (DEXTROSEPOTASSIUM CHLORIDESODIUM CHLORIDESODIUM LACTATE:Gastritis:0.5), (DIFLUCORTOLONE VALERATELIDOCAINE:Gastritis:0.58), (DIPHENHYDRAMINE HYDROCHLORIDELIDOCAINE HYDROCHLORIDENYSTATIN:Gastritis:0.11), (DOBUTAMINE HYDROCHLORIDE:Gastritis:1.01), (DOLASETRON MESYLATE:Gastritis:0.45), (DOXEPIN HYDROCHLORIDE:Gastritis:1.01), (DRONABINOL:Gastritis:1.63), (DUTASTERIDETAMS ULOSIN HYDROCHLORIDE:Gastritis:0.99), (ECONAZOLE:Gastritis:1.35), (ELBASVIRGRAZOPRE VIR:Gastritis:0.03), (ELETRIPTAN HYDROBROMIDE:Gastritis:1.97), (ELOTUZUMAB:Gastritis:1.15), (EMPAGLIFLOZINLINA GLIPTIN:Gastritis:1.05), (ENALAPRIL MALEATELERCANIDIPINE HYDROCHLORIDE:Gastritis:0.25), (EPHEDRA SINICA STEMHERBALS:Gastritis:0.27), (ERIBULIN:Gastritis:1.01), (ERYTHROMYCIN LACTOBIONATE:Gastritis:0.77), (ESTROGENS:Gastritis:0.27), (ETHACRYNIC ACID:Gastritis:0.21), (ETHINYL ESTRADIOLNORGESTIMATE:Gastritis:0.37), (FERRIC SODIUM CITRATE:Gastritis:0.4), (FERROUS SULFATEGLYCINE:Gastritis:1.14), (FLAX SEED:Gastritis:0.29), (FLOMOXEF:Gastritis:0.11), (FLOMOXEF SODIUM:Gastritis:0.07), (FLUNARIZINE HYDROCHLORIDE:Gastritis:0.03), (FLUPHENAZINE:Gastritis:0.32), (FLURAZEPAMFLUR AZEPAM HYDROCHLORIDE:Gastritis:0.48), (FLUTICASONE PROPIONATEFORMOTEROL FUMARATE:Gastritis:1.01), (FORMOTEROL FUMARATEMOMETASONE FUROATE:Gastritis:1.03), (FOSFOMYCIN:Gastritis:0.53), (FURSULTIAMINE:Gastritis:1.62), (GARLIC:Gastritis:0.43), (GLUCAGON:Gastritis:0.22), (GLYCOPYRROLATEINDACATERO L MALEATE:Gastritis:0.83), (GUANABENZ ACETATE:Gastritis:1.52), (HYDROCHLOROTHIAZIDELOSARTAN:Gastritis:0.22), (HYDR OCHLOROTHIAZIDEMETOPROLOL SUCCINATE:Gastritis:0.57), (HYDROCHLOROTHIAZIDEQUINAPRIL HYDROCHLORIDE:Gastritis:0.16), (HYDROCODONE BITARTRATEIBUPROFEN:Gastritis:0.45), (HYDROCORTISONE ACETATE:Gastritis:0.94), (HYDROCORTISONE SODIUM PHOSPHATE:Gastritis:0.01), (IMIGLUCERASE:Gastritis:0.85), (IMIQUIMOD:Gastritis:0.2), (I MMUNE GLOBULIN NOS:Gastritis:0.82), (INDAPAMIDEPERINDOPRIL:Gastritis:2.51), (INFLIXIMAB-DYYB:Gastritis:0.73), (INOTUZUMAB OZOGAMICIN:Gastritis:0.28), (INSULIN ASPARTINSULIN DEGLUDEC:Gastritis:0.65), (INTERFERON:Gastritis:1.49), (INTERFERON ALFA-2A OR INTERFERON ALFA-2B:Gastritis:0.05), (INTERFERON ALFA-N1:Gastritis:0.25), (INTERFERON BETA:Gastritis:0.06), (IPRAGLIFLOZIN:Gastritis:0.02), (IRBESARTANTRICHLORMETHIA ZIDE:Gastritis:0.1), (LABETALOLLABETALOL HYDROCHLORIDE:Gastritis:2.45), (LACTASE:Gastritis:0.33), (LANREOTIDE:Gastritis:0.35), (LEVALBUTEROL:Gastritis:0.37), (LEVOMEFOLATE CALCIUMMETHYLCOBALAMINPYRIDOXAL PHOSPHATE ANHYDROUS:Gastritis:0.35), (LINSIDOMINE:Gastritis:0.4), (LIOTHYRONINE SODIUM:Gastritis:1.08), (LOMITAPIDE MESYLATE:Gastritis:0.8), (LOTEPREDNOL ETABONATE:Gastritis:0.49), (LUTEIN:Gastritis:0.67), (LUTETIUM LU-177:Gastritis:0.26), (LYSOZYME HYDROCHLORIDE:Gastritis:1.64), (MAGNESIUM ASPARTATE HYDROCHLORIDE:Gastritis:0.35), (MELPERONE:Gastritis:0.61), (MEPENZOLATE BROMIDE:Gastritis:0.75), (MEPOLIZUMAB:Gastritis:0.98), (METHENAMINE HIPPURATE:Gastritis:1.42), (METHENOLONE ACETATE:Gastritis:0.2), (METHYLPHENIDATE:Gastritis:0.13), (METRELEPTIN:Gastritis:0.34), (MIGLUSTAT:Gastritis:0.21), (MIRIPLATIN:Gastritis:0.31), (MOEXIPRIL HYDROCHLORIDE:Gastritis:0.67), (NALOXONE:Gastritis:0.16), (NARATRIPTAN HYDROCHLORIDE:Gastritis:0.15), (NICARDIPINE HYDROCHLORIDE:Gastritis:1.08), (OCTOTIAMINE:Gastritis:0.06), (OK-432:Gastritis:0.31), (ORPHENADRINE:Gastritis:0.44), (OSELTAMIVIR:Gastritis:0.37), (OSPE MIFENE:Gastritis:0.34), (PATIROMER:Gastritis:0.64), (PEGINTERFERON ALFA-2A OR PEGINTERFERON ALFA-2B OR PEGINTERFERON BETA-1A OR PEGINTERFERON LAMBDA-1A:Gastritis:0.36), (PEGVISOMANT:Gastritis:0.38), (PENICILLIN G:Gastritis:0.69), (PENTOSTATIN:Gastritis:0.41), (PEROSPIRONE:Gastritis:0.13), (PEROSPIR ONE HYDROCHLORIDE:Gastritis:0.6), (PERPHENAZINE:Gastritis:0.06), (PHOSPHOLIPID:Gastritis: 0.05), (PILOCARPINE:Gastritis:0.77), (PILOCARPINE HYDROCHLORIDE:Gastritis:0.07), (PIRITRAMIDE:Gastritis:0.36), (POLYETHYLENE GLYCOL 3350\POTASSIUM CHLORIDESODIUM BICARBONATESODIUM CHLORIDESODIUM SULFATE ANHYDROUS:Gastritis:0.39), (POLYSTYRENE SULFONIC ACID:Gastritis:1.05), (PORFIMER SODIUM:Gastritis:0.09), (POTASSIUM CHLORIDESODIUM CHLORIDESODIUM LACTATE:Gastritis:1.11), (POTASSIUM CITRATETRISODIUM CITRATE DIHYDRATE:Gastritis:2.03), (PRASUGREL:Gastritis:0.09), (PREDNISOLONE SODIUM PHOSPHATE:Gastritis:0.84), (PRISTINAMYCIN:Gastritis:0.83), (PROBUCOL:Gastritis:1.32), (QUERCUS SALICINA STEM:Gastritis:0.38), (QUINACRINE:Gastritis:0.64), (QUININE:Gastritis:0.01), (RADIATION THERAPY:Gastritis:1.53), (RANIMUSTINE:Gastritis:0.01), (RHUBARB:Gastritis:0.48), (RILPI VIRINE HYDROCHLORIDE:Gastritis:1.15), (RISEDRONIC ACID:Gastritis:0.13), (SACCHAROMYCES CEREVISIAE:Gastritis:0.69), (SARPOGRELATE HYDROCHLORIDE:Gastritis:1.59), (SCOPOLAMINE HYDROBROMIDE:Gastritis:0.23), (SELEXIPAG:Gastritis:0.69), (SENNOSIDE ASENNOSIDE BSENNOSIDES:Gastritis:0.01), (SENNOSIDE B:Gastritis:0.33), (SERRAPEPTASE:Gastritis:1.03), (SILVER SULFADIAZINE:Gastritis:0.71), (SIVELESTAT SODIUM:Gastritis:0.64), (SOLIFENACIN:Gastritis:0.29), (SORBITOL:Gastritis:0.56), (STARC H, WHEAT:Gastritis:0.17), (STREPTOMYCINSTREPTOMYCIN SULFATE:Gastritis:0.98), (SUNITINIB:Gastritis:0.04), (SUPLATAST TOSILATE:Gastritis:1.89), (TAFLUPROST:Gastritis:0.19), (TANDOSPIRONE CITRATE:Gastritis:0.01), (TECELEUKIN:Gastritis:0.1), (TEDUGLUTIDEWATER:Gastritis:0.18), (TELOTRISTAT ETHYL:Gastritis:0.23), (TENOFOVIR:Gastritis:0.25), (TERBUTALINE:Gastritis:0.62), (TESTO STERONE UNDECANOATE:Gastritis:1.12), (THEOPHYLLINE:Gastritis:0.23), (THYROID, PORCINE:Gastritis:0.61), (TIMOLOLTRAVOPROST:Gastritis:0.06), (TOFISOPAM:Gastritis:00.85), (TRABECTEDIN:Gastritis:0.03), (TRANILAST:Gastritis:0.02), (TRIMETAZIDINE:Gastritis: 0.13), (TRIMIPRAMINE MALEATE:Gastritis:0.98), (TRIPTORELIN PAMOATE:Gastritis:0.37), (UMECLIDINIUM BROMIDE:Gastritis:0.68), (UROKINASE:Gastritis:0.72), (VALGANCICLOVIR:Gastritis:0.38), (VASOPRESSIN:Gastritis:0.03), (VITAMIN A:Gastritis:1.27), (VONOPRAZAN:Gastritis:l), (ZILEUTON:Gastritis:0.41), (DIGOXIN:Gastritis erosive:0), (EVOLOCUMAB:Gastritis erosive:0.13), (ACEBUTOLOL HYDROCHLORIDE:Gastritis prophylaxis:0.23), (ACETAMINOPHENAPRONALIDECAFFEINEPROPYPHENAZONE:Ga stritis prophylaxis:0.79), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Gastritis prophylaxis:0.1), (ACETAMINOPHENBUTALBITALCAFFEINE:Gastritis prophylaxis:0.73), (ACETAMINOPHENHYDROCODONE:Gastritis prophylaxis:0.77), (ACETAMINOPHENOXYCODONEOXYCODONE HYDROCHLORIDE:Gastritis prophylaxis:0.62), (ACLARUBICIN HYDROCHLORIDE:Gastritis prophylaxis:0.01), (ADENINE:Gastritis prophylaxis:1.21), (AGALSIDASE BETA:Gastritis prophylaxis:0.22), (ALBUTEROLIPRATROPIUM:Gastritis prophylaxis:0.19), (ALDESLEUKIN:Gastritis prophylaxis:0.12), (ALECTINIB:Gastritis prophylaxis:0.28), (ALUMINUM HYDROXIDEMAGNESIUM OXIDEOXETHAZAINE:Gastritis prophylaxis:0.03), (ALUMINUM SILICATE:Gastritis prophylaxis:1.1), (AMANTADINE:Gastritis prophylaxis:0.53), (AMLODIPINEPERINDOPRIL:Gastritis prophylaxis:0.62), (AMPHETAMINE ASPARTATEAMPHETAMINE SULFATEDEXTROAMPHETAMINE SACCHARATEDEXTROAMPHETAMINE SULFATE:Gastritis prophylaxis:0.26), (AMPICILLIN SODIUM:Gastritis prophylaxis:0.29), (ANAGLIPTIN:Gastritis prophylaxis:0.96), (ANAGRELIDE HYDROCHLORIDE:Gastritis prophylaxis:0.26), (ANTIHISTAMINES NOS:Gastritis prophylaxis:0), (APRINDINE HYDROCHLORIDE:Gastritis prophylaxis:0.42),(ARMODAFINIL:Gastritis prophylaxis:0.36), (ASPIRINMAGNESIUM OXIDE:Gastritis prophylaxis:0.67), (BACITRACIN:Gastritis prophylaxis:0.02), (BENPROPERINE PHOSPHATE:Gastritis prophylaxis:0.45), (BETAMETHASONE DIPROPIONATECALCIPOTRIENE:Gastritis prophylaxis:0.18), (BETAMETHASONE SODIUM PHOSPHATENEOMYCIN SULFATE:Gastritis prophylaxis:1.08), (BICARBONATE IONPOLYETHYLENE GLYCOLSPOTASSIUM CATIONSODIUM CHLORIDESODIUM SULFATE ANHYDROUS:Gastritis prophylaxis:0.38), (BILASTINE:Gastritis prophylaxis:0.41), (BIXALOMER:Gastritis prophylaxis:0.28), (BLESELUMAB:Gastritis prophylaxis:0.19), (BLINATUMOMAB:Gastritis prophylaxis:0.89), (BROMHEXINE:Gastritis prophylaxis:0.19), (BUCOLOME:Gastritis prophylaxis:0.05), (BUDESONIDEFORMOTEROL:Gastritis prophylaxis:0.06), (BUFORMIN:Gastritis prophylaxis:0.25), (BUPRENORPHINE HYDROCHLORIDENALOXONE HYDROCHLORIDE:Gastritis prophylaxis:0.02), (CALCIPOTRIENE:Gastritis prophylaxis:0.18), (CALCITONIN:Gastritis prophylaxis:0.46), (CALCIUM CARBONATEERGOCALCIFEROLRETINOL:Gastritis prophylaxis:0.32), (CALCIUM CITRATE:Gastritis prophylaxis:0.5), (CALCIUMMAGNESIUMZINC:Gastritis prophylaxis:0.01), (CANAGLIFLOZINMETFORMIN HYDROCHLORIDE:Gastritis prophylaxis:0.56), (CEFCAPENE PIVOXIL:Gastritis prophylaxis:0.1), (CEFMETAZOLE:Gastritis prophylaxis:0.3), (CEFOTAXIMECEFOTAXIME SODIUM:Gastritis prophylaxis:0.22), (CEFOTIAM:Gastritis prophylaxis:0.2), (CEFPIROME SULFATE:Gastritis prophylaxis:0.67), (CERTOPARIN SODIUM:Gastritis prophylaxis:0.67), (CHOLESTYRAMINE:Gastritis prophylaxis:1.51), (CHOLINE BITARTRATECYSTEINEINOSITOLMAMMAL LIVER:Gastritis prophylaxis:0.43), (CHONDROITIN SULFATE (BOVINE)GLUCOSAMINE HYDROCHLORIDE:Gastritis prophylaxis:0.01), (CIFENLINE:Gastritis prophylaxis:0.96), (CLINDAMYCIN:Gastritis prophylaxis:1.64), (CUPRIC SULFATEFERRIC CHLORIDEMANGANESE CHLORIDEPOTASSIUM IODIDEZINC SULFATE:Gastritis prophylaxis:0.27), (CYAMEMAZINE:Gastritis prophylaxis:0.14), (CYANAMIDE:Gastritis prophylaxis:0.05), (CYANOCOBALAMINLIDOCAINE HYDROCHLORIDEPYRIDOXINE HYDROCHLORIDETHIAMINE HYDROCHLORIDE:Gastritis prophylaxis:0.43), (CYCLOBENZAPRINE:Gastritis prophylaxis:0.52), (CYTARABINE OCFOSFATE:Gastritis prophylaxis:0.02), (DACLIZUMAB:Gastritis prophylaxis:1.01), (DAPAGLIFLOZIN PROPANEDIOL:Gastritis prophylaxis:0.14), (DEFERIPRONE:Gastritis prophylaxis:0.41), (DESLORATADINE:Gastritis prophylaxis:1.29), (DESMOPRESSIN:Gastritis prophylaxis:0.05), (DESVENLAFAXINE SUCCINATE:Gastritis prophylaxis:0.24), (DEVICE:Gastritis prophylaxis:0.24), (DEXTROSEPOTASSIUM CHLORIDESODIUM CHLORIDESODIUM LACTATE:Gastritis prophylaxis:0.34), (DIAZOXIDE:Gastritis prophylaxis:0.27), (DIFLUCORTOLONE VALERATELIDOCAINE:Gastritis prophylaxis:0.26), (DISOPYRAMIDE:Gastritis prophylaxis:0.3), (DIVALPROEX SODIUM:Gastritis prophylaxis:1.31), (DOBUTAMINEDOBUTAMINE HYDROCHLORIDE:Gastritis prophylaxis:0.24), (DOCUSATE CALCIUM:Gastritis prophylaxis:0.59), (DOFETILIDE:Gastritis prophylaxis:0.44), (DOLASETRON MESYLATE:Gastritis prophylaxis:0.11), (DONEPEZIL:Gastritis prophylaxis:0.26), (DORIPENEM:Gastritis prophylaxis:0.48), (DOXEPIN HYDROCHLORIDE:Gastritis prophylaxis:0.16), (ELBASVIR:Gastritis prophylaxis:0.22), (ELCATONIN:Gastritis prophylaxis:0.55), (EMPAGLIFLOZINLINAGLIPTIN:Gastritis prophylaxis:0.22), (EPHEDRA SINICA STEMHERBALS:Gastritis prophylaxis:0.52), (ERYTHROMYCIN:Gastritis prophylaxis:1.1), (ESTROGENS, CONJUGATEDMEDROXYPROGESTERONE ACETATE:Gastritis prophylaxis:0.17), (ETHYL ICOSAPENTATEICOSAPENT:Gastritis prophylaxis:1.32), (ETIMICIN SULFATE:Gastritis prophylaxis:1.31), (ETRETINATE:Gastritis prophylaxis:1.88), (EVOLOCUMAB:Gastritis prophylaxis:0.97), (FENOFIBRIC ACID:Gastritis prophylaxis:0.05), (FERRIC HYDROXIDE:Gastritis prophylaxis:0.32), (FERRIC PYROPHOSPHATE:Gastritis prophylaxis:0.11), (FERRIC SODIUM CITRATE:Gastritis prophylaxis:0.2), (FERROUS GLUCONATE:Gastritis prophylaxis:0.08), (FLOMOXEF:Gastritis prophylaxis:0.46), (FLOPROPIONE:Gastritis prophylaxis:0.14), (FLUINDIONE:Gastritis prophylaxis:0.87), (FLUOCINONIDE:Gastritis prophylaxis:0.33), (FLUTAMIDE:Gastritis prophylaxis:0.11), (FLUTICASONE:Gastritis prophylaxis:0.72), (FORMOTEROL:Gastritis prophylaxis:0.98), (FOSFOMYCIN CALCIUM:Gastritis prophylaxis:0.74), (FURSULTIAMINE:Gastritis prophylaxis:1.37), (GARENOXACIN:Gastritis prophylaxis:0.3), (GLYCOPYRROLATE:Gastritis prophylaxis:0.38), (GLYCOPYRROLATEINDACATEROL MALEATE:Gastritis prophylaxis:0.17), (GLYCOPYRRONIUM:Gastritis prophylaxis:0.14), (GRAZOPREVIR:Gastritis prophylaxis:0.19), (GUAIAZULENE:Gastritis prophylaxis:0.14), (GUAIFENESIN:Gastritis prophylaxis:0.7), (GUSPERIMUS TRIHYDROCHLORIDE:Gastritis prophylaxis:0.35), (HERBALSTURMERIC:Gastritis prophylaxis:0.42), (HYDROCHLOROTHIAZIDETELMISARTAN:Gastritis prophylaxis:1.01), (HYDROCORTISONE ACETATE:Gastritis prophylaxis:0.62), (HYDROXYCHLOROQUINE DIPHOSPHATE:Gastritis prophylaxis:0.27), (HYDROXYZINE PAMOATE:Gastritis prophylaxis:1.41), (HYDROXYZINEHYDROXYZINE HYDROCHLORIDE:Gastritis prophylaxis:0.24), (IBRITUMOMAB TIUXETAN:Gastritis prophylaxis:1.08), (IBUDILAST:Gastritis prophylaxis:0.42), (IFENPRODIL:Gastritis prophylaxis:0.6), (IFENPRODIL TARTRATE:Gastritis prophylaxis:0.26), (IMIDAPRIL HYDROCHLORIDE:Gastritis prophylaxis:0.75), (IMIGLUCERASE:Gastritis prophylaxis:0.03), (INSULIN ASPARTINSULIN DEGLUDEC:Gastritis prophylaxis:0.07), (INTERFERON:Gastritis prophylaxis:0.35), (INTERFERON ALFA-2A:Gastritis prophylaxis:0.46), (INTERFERON ALFA-2B:Gastritis prophylaxis:0.49), (INTERFERON ALFA-N1:Gastritis prophylaxis:0.06), (INVERT SUGAR:Gastritis prophylaxis:0.15), (IODINE:Gastritis prophylaxis:0.02), (IOPAMIDOL:Gastritis prophylaxis:0.22), (IPRAGLIFLOZIN L-PROLINE:Gastritis prophylaxis:0.35), (KALLIDINOGENASE:Gastritis prophylaxis:0.73), (LABETALOL HYDROCHLORIDE:Gastritis prophylaxis:0.82), (LABETALOLLABETALOL HYDROCHLORIDE:Gastritis prophylaxis:1.57), (LEVALBUTEROL HYDROCHLORIDE:Gastritis prophylaxis:0.9), (LEVOCABASTINE HYDROCHLORIDE:Gastritis prophylaxis:1.19), (LICORICE ROOT EXTRACTRHUBARB:Gastritis prophylaxis:0.3), (LINSIDOMINE:Gastritis prophylaxis:0), (LITHIUM:Gastritis prophylaxis:0.02), (MAGNESIUM ASPARTATE HYDROCHLORIDE:Gastritis prophylaxis:0.2), (MANIDIPINE HYDROCHLORIDE:Gastritis prophylaxis:0.09), (MECLIZINE HYDROCHLORIDE:Gastritis prophylaxis:0.34), (METAXALONE:Gastritis prophylaxis:0.17), (METFORMIN PAMOATE:Gastritis prophylaxis:0.22), (METHENAMINE HIPPURATE:Gastritis prophylaxis:0.65), (METHENOLONE ACETATE:Gastritis prophylaxis:0.68), (METHOCARBAMOL:Gastritis prophylaxis:0.8), (METHYLPHENIDATE HYDROCHLORIDE:Gastritis prophylaxis:0.42), (METHYLPREDNISOLONE ACETATE:Gastritis prophylaxis:1.29), (METOLAZONE:Gastritis prophylaxis:0.39), (NADOLOL:Gastritis prophylaxis:0.33), (NALFURAFINE HYDROCHLORIDE:Gastritis prophylaxis:0.28), (NIACIN:Gastritis prophylaxis:0.86), (NILVADIPINE:Gastritis prophylaxis:0.3), (NIRAPARIB:Gastritis prophylaxis:0.29), (NITROFURANTOINNITROFURANTOIN MONOHYDRATE:Gastritis prophylaxis:0.01), (NOREPINEPHRINE BITARTRATE:Gastritis prophylaxis:0.24), (OCTOTIAMINE:Gastritis prophylaxis:0.21), (OK-432:Gastritis prophylaxis:0.29), (ORGOTEIN:Gastritis prophylaxis:0.02), (PANTOPRAZOLE MAGNESIUM:Gastritis prophylaxis:1.29), (PAPAVERINE HYDROCHLORIDE:Gastritis prophylaxis:0.3), (PARICALCITOL:Gastritis prophylaxis:0.04), (PAZUFLOXACIN MESILATE:Gastritis prophylaxis:0.5), (PEGASPARGASE:Gastritis prophylaxis:0.49), (PEGZEREPOETIN ALFA:Gastritis prophylaxis:0.75), (PENTOSTATIN:Gastritis prophylaxis:0.54), (PERAMIVIR:Gastritis prophylaxis:0.09), (PHENOTHIAZINE:Gastritis prophylaxis:0.12), (PIRITRAMIDE:Gastritis prophylaxis:0.33), (POLYSTYRENE:Gastritis prophylaxis:0.86), (PORFIMER SODIUM:Gastritis prophylaxis:0.63), (PREDNISOLONE SODIUM PHOSPHATE:Gastritis prophylaxis:0.6), (PRIMIDONE:Gastritis prophylaxis:1.36), (PROPAFENONE:Gastritis prophylaxis:0.48), (PSYLLIUM HUSK:Gastritis prophylaxis:0.16), (PYRIDOXAL PHOSPHATE:Gastritis prophylaxis:0.32), (QUAZEPAM:Gastritis prophylaxis:0.22), (QUERCUS SALICINA STEM:Gastritis prophylaxis:0.4), (QUINACRINE:Gastritis prophylaxis:0.05), (RADIATION THERAPY:Gastritis prophylaxis:1.27), (RANIMUSTINE:Gastritis prophylaxis:0.41), (RETINOL:Gastritis prophylaxis:0.36), (RILMENIDINE PHOSPHATE:Gastritis prophylaxis:0.67), (RIOCIGUAT:Gastritis prophylaxis:0.42), (ROFLUMILAST:Gastritis prophylaxis:0.99), (SALMETEROL:Gastritis prophylaxis:0.22), (SENNOSIDE A:Gastritis prophylaxis:0.13), (SENNOSIDE B:Gastritis prophylaxis:0.91), (SERRAPEPTASE:Gastritis prophylaxis:0.47), (SEVELAMER CARBONATE:Gastritis prophylaxis:0.24), (SILVER SULFADIAZINE:Gastritis prophylaxis:0.05), (SITAFLOXACIN:Gastritis prophylaxis:0.87), (SODIUM CITRATE:Gastritis prophylaxis:0.13), (SODIUM FLUORIDE:Gastritis prophylaxis:0.21), (SODIUM GUALENATE MONOHYDRATE:Gastritis prophylaxis:0.66), (SORBITOL:Gastritis prophylaxis:0.25), (SOTALOL HYDROCHLORIDE:Gastritis prophylaxis:0.29), (STREPTOCOCCUS FAECALIS:Gastritis prophylaxis:0.11), (SULBACTAM:Gastritis prophylaxis:0.25), (TECELEUKIN:Gastritis prophylaxis:0.15), (TEGAFURURACIL:Gastritis prophylaxis:0.36), (TEMSIROLIMUS:Gastritis prophylaxis:0.07), (TERBUTALINE:Gastritis prophylaxis:0.75), (TESTOSTERONE UNDECANOATE:Gastritis prophylaxis:0.24), (TETRACYCLINE:Gastritis prophylaxis:0.1), (THIAMYLAL SODIUM:Gastritis prophylaxis:0.6), (THIOCTIC ACID:Gastritis prophylaxis:0.14), (TIARAMIDE:Gastritis prophylaxis:0.09), (TIMOLOL MALEATE:Gastritis prophylaxis:0.36), (TOPIROXOSTAT:Gastritis prophylaxis:0.36), (TRABECTEDIN:Gastritis prophylaxis:0.02), (TRIMIPRAMINE MALEATE:Gastritis prophylaxis:0.19), (TULOBUTEROL HYDROCHLORIDE:Gastritis prophylaxis:0.05), (UROKINASE:Gastritis prophylaxis:1.11), (VASOPRESSIN:Gastritis prophylaxis:0.03), (VINDESINE SULFATE:Gastritis prophylaxis:0.54), (ZINC:Gastritis prophylaxis:0.44), (ZIV-AFLIBERCEPT:Gastritis prophylaxis:0.41), (ACETAMINOPHENCODEINE PHOSPHATE:Gastroenteritis:0.51), (AMEZINIUM METILSULFATE:Gastroenteritis:0.53), (AMLODIPINE BESYLATETELMISARTAN:Gastroenteritis:0.01), (ANASTROZOLE:Gastroenteritis:0.47), (A ZELNIDIPINE:Gastroenteritis:0.36), (BUMETANIDE:Gastroenteritis:0.61), (CALCIUM CARBONATECHOLECALCIFEROL:Gastroenteritis:0.81), (CAMPHORMENTHOLMETHY L SALICYLATE:Gastroenteritis:0.14), (CANDESARTAN CILEXETILHYDROCHLOROTHIAZIDE:Gastroenteritis:0.07), (CARFILZOMIB:Gastroenteritis: 0.13), (CEFCAPENE PIVOXIL HYDROCHLORIDE:Gastroenteritis:0.36), (CHOLESTYRAMINE:Gastroenteritis:0.51), (CLOT RIMAZOLE:Gastroenteritis:0.21), (COLCHICINE:Gastroenteritis:0.3), (CORTISONEHYDRO CORTISONE:Gastroenteritis:0.08), (DAPSONE:Gastroenteritis:0.3), (DASATINIB:Gastroenteritis: 0.51), (DEXAMETHASONE ACETATE:Gastroenteritis:0.83), (DILAZEP:Gastroenteritis:0.83), (DIPHENIDOL HYDROCHLORIDE:Gastroenteritis:0.41), (ERGOCALCIFEROL:Gastroenteritis:0.2), (ETRETI NATE:Gastroenteritis:0.28), (EVOLOCUMAB:Gastroenteritis:0.17), (FERROUS SULFATEFERROUS SULFATE, DRIED:Gastroenteritis:0.89), (FISH OIL:Gastroenteritis:0.6), (FLUTICASONE:Gastroenteritis:0.02), (FLUTICASONE PROPIONATE:Gastroenteritis:1.26), (GLIPIZIDE:Gastroenteritis:0.21), (HALOPERIDOL:Gastroenteritis: 0.14), (HEPARIN SODIUM:Gastroenteritis:0.36), (HYDROCHLOROTHIAZIDEIRBESARTAN:Gastroenteritis:001), (HYDROCHLOROTHIAZIDELISINOPRIL:Gastroenteritis:0.22), (HYDROXYUREA: Gastroenteritis:0.12), (IGURATIMOD:Gastroenteritis:0.07), (ILOPROST:Gastroenteritis:0.04), (IMA TINIB MESYLATE:Gastroenteritis:0.3), (IMMUNE GLOBULIN NOS:Gastroenteritis:0.22), (INDAPAMIDEPERINDOPRIL:Gastroenteritis:0.1), (INDOMETHA CIN:Gastroenteritis:0.06), (INSULIN HUMAN:Gastroenteritis:0.25), (ISOSORBIDE MONONITRATE:Gastroenteritis:0.04), (LERCANIDIPINE HYDROCHLORIDE:Gastroenteritis:0.04), (LEVOCABASTINE HYDROCHLORIDE:Gastroenteritis:0.1), (LIDOCAINE:Gastroenteritis:0.32), (LYSOZYME HYDROCHLORIDE:Gastroenteritis:0.16), (MAXACALCITOL:Gastroenteritis:0.07), (METOPR OLOL TARTRATE:Gastroenteritis:0.31), (NEBIVOLOL:Gastroenteritis:0.49), (OXCARBAZEPINE: Gastroenteritis:0.04), (PANTOPRAZOLE MAGNESIUM:Gastroenteritis:0.2), (POTASSIUM CITRATETRISODIUM CITRATE DIHYDRATE:Gastroenteritis:0.18), (QUETIAPINE:Gastroenteritis:0.13), (RIOCIGUAT:Gastroenteritis: 0.38), (SACUBITRILVALSARTAN:Gastroenteritis:0.47), (SECUKINUMAB:Gastroenteritis: 0.61), (SOTALOL:Gastroenteritis:0.12), (TERIFLUNOMIDE:Gastroenteritis:0.02), (TIPEPI DINE HIBENZATE:Gastroenteritis:0.06), (TOFACITINIB CITRATE:Gastroenteritis:0.28), (TOPIRAMATE:Gastroenteritis:0.95), (VANCOMYCIN HYDROCHLORIDE:Gastroenteritis:0.08), (VARENICLINE TARTRATE:Gastroenteritis:0.06), (ZOLPIDEMZOLPIDEM TARTRATE:Gastroenteritis:0.39), (LEVOLEUCOVORIN CALCIUM:Gastrointestinal carcinoma:0.17), (.ALPHA.-TOCOPHEROL ACETATE, DL-ASCORBIC ACIDCYANOCOBALAMINFLUORIDE IONFOLIC ACIDNIACINPYRIDOXINERIBOFLAVINTHIAMINEVITAMIN AVITAMIN D:Gastrointestinal disorder:0.6), (ABOBOTULINUMTOXINA:Gastrointestinal disorder:0.02), (ACETAMINOPHENCAFFEINEOPIUM:Gastrointestinal disorder:0.04), (ACETAMINOPHENOXYCODONEOXYCODONE HYDROCHLORIDE:Gastrointestinal disorder:1.75), (ACETAMINOPHENPROPOXYPHENE HYDROCHLORIDE:Gastrointestinal disorder:0.83), (ACETAMINOPHENPROPOXYPHENE NAPSYLATE:Gastrointestinal disorder:0.39), (ADO-TRASTUZUMAB EMTANSINE:Gastrointestinal disorder:1.27), (ALBIGLUTIDE:Gastrointestinal disorder:0.19), (ALISKIREN:Gastrointestinal disorder:0.89), (AMINO ACIDSDEXTROSEELECTROLYTES NOS:Gastrointestinal disorder:1.31), (AMLODIPINE BESYLATEOLMESARTAN MEDOXOMIL:Gastrointestinal disorder:0.85), (AMMONIUM CHLORIDEDEXTROMETHORPHANSODIUM CITRATE:Gastrointestinal disorder:0.18), (AMOXICILLINCLARITHROMYCINESOMEPRAZOLE:Gastrointestinal disorder:0.42), (ANAGRELIDE HYDROCHLORIDE:Gastrointestinal disorder:0.35), (ANTIHISTAMINES NOS:Gastrointestinal disorder:0.48), (ARIPIPRAZOLE LAUROXIL:Gastrointestinal disorder:0.06), (ASCORBIC ACIDBIOTINCALCIUM PANTOTHENATECOBALAMINFOLIC ACIDNIACINAMIDEPYRIDOXINE HYDROCHLORIDERIBOFLAVINTHIAMINE MONONITRATE:Gastrointestinal disorder:0.31), (ASPIRINMAGNESIUM OXIDE:Gastrointestinal disorder:0.3), (ATROPINE SULFATE:Gastrointestinal disorder:0.4), (AZOSEMIDE:Gastrointestinal disorder:0.74), (BECLOMETHASONEFORMOTEROL:Gastrointestinal disorder:0.1), (BELATACEPT:Gastrointestinal disorder:0.42), (BENDAMUSTINE HYDROCHLORIDE:Gastrointestinal disorder:0.29), (BETAMETHASONE DIPROPIONATECALCIPOTRIENECALCIPOTRIENE HYDRATE:Gastrointestinal disorder:0.79), (BETAMETHASONE DIPROPIONATECLOTRIMAZOLE:Gastrointestinal disorder:0.58), (BETAXOLOL HYDROCHLORIDE:Gastrointestinal disorder:0.02), (BICARBONATE ION:Gastrointestinal disorder:0.17), (BILASTINE:Gastrointestinal disorder:0.45), (BLINATUMOMAB:Gastrointestinal disorder:0.4), (BOCEPREVIR:Gastrointestinal disorder:0.33), (BUDESONIDEFORMOTEROL:Gastrointestinal disorder:0.25), (BUPIVACAINE HYDROCHLORIDE:Gastrointestinal disorder:0.14), (BUPRENORPHINE HYDROCHLORIDE:Gastrointestinal disorder:1.23), (BUTALBITAL:Gastrointestinal disorder:0.09), (CALCIPOTRIENE:Gastrointestinal disorder:0.85), (CALCITONIN:Gastrointestinal disorder:1.04), (CALCIUM CITRATEVITAMIN D:Gastrointestinal disorder:0.25), (CALCIUM PANTOTHENATECYANOCOBALAMINNIACINAMIDEPYRIDOXINE HYDROCHLORIDERIBOFLAVINTHIAMINE MONONITRATE:Gastrointestinal disorder:0.44), (CALCIUMMAGNESIUMZINC:Gastrointestinal disorder:0.86), (CANAGLIFLOZINMETFORMIN HYDROCHLORIDE:Gastrointestinal disorder:1.28), (CAPSAICIN:Gastrointestinal disorder:0.31), (CARBIMAZOLE:Gastrointestinal disorder:0.79), (CARTEOLOL HYDROCHLORIDE:Gastrointestinal disorder:0), (CASPOFUNGIN:Gastrointestinal disorder:0.37), (CEFUROXIME:Gastrointestinal disorder:0.86), (CEFUROXIME AXETIL:Gastrointestinal disorder:0.43), (CERIVASTATIN SODIUM:Gastrointestinal disorder:0.04), (CHLORAMBUCIL:Gastrointestinal disorder:0.41), (CHLORHEXIDINE:Gastrointestinal disorder:0.58), (CHLOROTHIAZIDE:Gastrointestinal disorder:0.24), (CILASTATIN SODIUMIMIPENEM:Gastrointestinal disorder:1.52), (CILNIDIPINE:Gastrointestinal disorder:0.13), (CLOMIPRAMINE HYDROCHLORIDE:Gastrointestinal disorder:0.15), (COBICISTATELVITEGRAVIREMTRICITABINETENOFOVIR ALAFENAMIDE FUMARATE:Gastrointestinal disorder:0.11), (CORTICOTROPIN:Gastrointestinal disorder:0.98), (CYAMEMAZINE:Gastrointestinal disorder:0.06), (CYPROHEPTADINE HYDROCHLORIDE:Gastrointestinal disorder:0.44), (DABRAFENIB:Gastrointestinal disorder:0.52), (DABRAFENIB MESYLATE:Gastrointestinal disorder:0.2), (DACLIZUMAB:Gastrointestinal disorder:1.72), (DALTEPARIN:Gastrointestinal disorder:1.96), (DANAZOL:Gastrointestinal disorder:1.93), (DANTROLENE SODIUM:Gastrointestinal disorder:0.42), (DAPAGLIFLOZIN PROPANEDIOL:Gastrointestinal disorder:0.74), (DEGARELIX:Gastrointestinal disorder:0.11), (DEXTROAMPHETAMINE:Gastrointestinal disorder:0.09), (DEXTROMETHORPHAN HYDROBROMIDEQUINIDINE SULFATE:Gastrointestinal disorder:0.05), (DICLOFENAC SODIUMMISOPROSTOL:Gastrointestinal disorder:0.3), (DOBUTAMINE HYDROCHLORIDE:Gastrointestinal disorder:0.08), (DOLASETRON MESYLATE:Gastrointestinal disorder:0.47), (DOXYCYCLINE HYCLATE:Gastrointestinal disorder:0.94), (DROXIDOPA:Gastrointestinal disorder:0.18), (DUTASTERIDETAMSULOSIN HYDROCHLORIDE:Gastrointestinal disorder:0.93), (ECONAZOLE:Gastrointestinal disorder:1.48), (ELTROMBOPAG OLAMINE:Gastrointestinal disorder:0.47), (EMPAGLIFLOZINLINAGLIPTIN:Gastrointestinal disorder:0.74), (ENALAPRIL MALEATEHYDROCHLOROTHIAZIDE:Gastrointestinal disorder:0.23), (ERIBULIN:Gastrointestinal disorder:0.2), (ERLOTINIB:Gastrointestinal disorder:0.18), (ERTAPENEM SODIUM:Gastrointestinal disorder:0.05), (ETHACRYNIC ACID:Gastrointestinal disorder:0.12), (ETHINYL ESTRADIOLNORETHINDRONE:Gastrointestinal disorder:0.3), (ETHINYL ESTRADIOLNORGESTREL:Gastrointestinal disorder:0.25), (ETRETINATE:Gastrointestinal disorder:0.61), (FAMCICLOVIR:Gastrointestinal disorder:0.06), (FERROUS SULFATEGLYCINE:Gastrointestinal disorder:0.16), (FEXOFENADINEPSEUDOEPHEDRINE:Gastrointestinal disorder:0.96), (FLURAZEPAMFLURAZEPAM HYDROCHLORIDE:Gastrointestinal disorder:0.14), (FLURBIPROFEN:Gastrointestinal disorder:0.02), (FLUVOXAMINE MALEATE:Gastrointestinal disorder:1.28), (FORMOTEROL FUMARATEMOMETASONE FUROATE:Gastrointestinal disorder:1.03), (FOSFOMYCIN:Gastrointestinal disorder:0.09), (GLYCOPYRROLATEINDACATEROL MALEATE:Gastrointestinal disorder:0.44), (GOSERELIN ACETATE:Gastrointestinal disorder:0.13), (GRANULOCYTE COLONY-STIMULATING FACTOR NOS:Gastrointestinal disorder:0.31), (HYDROCODONE BITARTRATEIBUPROFEN:Gastrointestinal disorder:0.78), (HYDROXYCHLOROQUINE DIPHOSPHATE:Gastrointestinal disorder:0.99), (IDELALISIB:Gastrointestinal disorder:0.23), (IMIGLUCERASE:Gastrointestinal disorder:0.63), (IMMUNE GLOBULIN NOS:Gastrointestinal disorder:0.45), (INOTUZUMAB OZOGAMICIN:Gastrointestinal disorder:0.17), (INSULIN DEGLUDEC:Gastrointestinal disorder:0.5), (INTERFERON ALFA-2A:Gastrointestinal disorder:1.17), (INTERFERON ALFA-2B:Gastrointestinal disorder:0.77), (IOPAMIDOL:Gastrointestinal disorder:0.08), (IVACAFTOR:Gastrointestinal disorder:0.25), (LABETALOL HYDROCHLORIDE:Gastrointestinal disorder:1.73), (LACTOBACILLUS ACIDOPHILUSLACTOBACILLUS DELBRUECKII SUBSP. BULGARICUS:Gastrointestinal disorder:0.09), (LEVODOPA:Gastrointestinal disorder:0.27), (LEVOMILNACIPRAN HYDROCHLORIDE:Gastrointestinal disorder:0.13), (LINSIDOMINE:Gastrointestinal disorder:0.01), (LUTETIUM LU-177:Gastrointestinal disorder:0.04), (MEDROXYPROGESTERONE:Gastrointestinal disorder:0.44), (METFORMIN HYDROCHLORIDESAXAGLIPTIN HYDROCHLORIDE:Gastrointestinal disorder:0.19), (METHOXY POLYETHYLENE GLYCOL-EPOETIN BETA:Gastrointestinal disorder:0.17), (MICAFUNGIN:Gastrointestinal disorder:0.42), (MOXIFLOXACIN:Gastrointestinal disorder:1.27), (MOXONIDINE:Gastrointestinal disorder:0.86), (NADROPARIN CALCIUM:Gastrointestinal disorder:1.49), (NALOXONE HYDROCHLORIDETILIDINE HYDROCHLORIDE:Gastrointestinal disorder:1.67), (NARATRIPTAN HYDROCHLORIDE:Gastrointestinal disorder:0.22), (NEPAFENAC:Gastrointestinal disorder:0.16), (NIRAPARIB:Gastrointestinal disorder:1.14), (NOREPINEPHRINE:Gastrointestinal disorder:0.42), (NOREPINEPHRINE BITARTRATE:Gastrointestinal disorder:0.44), (OLOPATADINE:Gastrointestinal disorder:0.45), (ORPHENADRINE:Gastrointestinal disorder:0.48), (OSELTAMIVIR:Gastrointestinal disorder:0.57), (OSELTAMIVIR PHOSPHATE:Gastrointestinal disorder:0.68), (OSPEMIFENE:Gastrointestinal disorder:0.32), (OXAPROZIN:Gastrointestinal disorder:0.14), (OXYBUTYNINOXYBUTYNIN CHLORIDE:Gastrointestinal disorder:0.29), (OXYMETAZOLINE:Gastrointestinal disorder:0.39), (PAMIDRONATE DISODIUM:Gastrointestinal disorder:0.9), (PASIREOTIDE:Gastrointestinal disorder:0.2), (PEGINTERFERON BETA-1A:Gastrointestinal disorder:1.27), (PEMETREXED:Gastrointestinal disorder:0.93), (PEMETREXED DISODIUM:Gastrointestinal disorder:1.22), (PILSICAINIDE HYDROCHLORIDE:Gastrointestinal disorder:0.34), (PIRFENIDONE:Gastrointestinal disorder:0.11), (POLYETHYLENE GLYCOL 3350\POTASSIUM CHLORIDESODIUM BICARBONATESODIUM CHLORIDESODIUM SULFATE ANHYDROUS:Gastrointestinal disorder:0.57), (POLYETHYLENE GLYCOL 4000:Gastrointestinal disorder:0.78), (PRANLUKAST:Gastrointestinal disorder:0.2), (PRAZEPAM:Gastrointestinal disorder:0.02), (PREDNISOLONE SODIUM PHOSPHATE:Gastrointestinal disorder:0.67), (PRISTINAMYCIN:Gastrointestinal disorder:0.12), (PROTRIPTYLINE HYDROCHLORIDE:Gastrointestinal disorder:0.46), (QUINACRINE:Gastrointestinal disorder:0.42), (RILMENIDINE PHOSPHATE:Gastrointestinal disorder:0.22), (RILPIVIRINE HYDROCHLORIDE:Gastrointestinal disorder:0.26), (ROFECOXIB:Gastrointestinal disorder:0.03), (SARPOGRELATE:Gastrointestinal disorder:0.47), (SELEGILINE:Gastrointestinal disorder:0.23), (SELEXIPAG:Gastrointestinal disorder:0.52), (SEVELAMER CARBONATE:Gastrointestinal disorder:0.92), (SIMEPREVIR:Gastrointestinal disorder:0.03), (SIPULEUCEL-T:Gastrointestinal disorder:0.32), (SODIUM PHOSPHATE, DIBASICSODIUM PHOSPHATE, MONOBASIC:Gastrointestinal disorder:0.87), (SOFOSBUVIRVELPATASVIR:Gastrointestinal disorder:0.37), (SORBITOL:Gastrointestinal disorder:0), (SUFENTANIL CITRATE:Gastrointestinal disorder:0.06), (TAMOXIFEN CITRATE:Gastrointestinal disorder:0.06), (TASIMELTEON:Gastrointestinal disorder:0.02), (TELOTRISTAT ETHYL:Gastrointestinal disorder:0.4), (TERBUTALINE:Gastrointestinal disorder:0.28), (TESTOSTERONE UNDECANOATE:Gastrointestinal disorder:0.79), (TETRACYCLINE:Gastrointestinal disorder:1.68), (THEOPHYLLINE:Gastrointestinal disorder:0.38), (TIGECYCLINE:Gastrointestinal disorder:0.32), (TRAMETINIB:Gastrointestinal disorder:0.5), (TRAMETINIB DIMETHYL SULFOXIDE:Gastrointestinal disorder:0.16), (TRIPTORELIN PAMOATE:Gastrointestinal disorder:0.18), (UMECLIDINIUM BROMIDE:Gastrointestinal disorder:0.83), (VARDENAFIL HYDROCHLORIDE TRIHYDRATE:Gastrointestinal disorder:0.17), (VINORELBINE TARTRATE:Gastrointestinal disorder:0.59), (YEAST:Gastrointestinal disorder:0.26), (ZAFIRLUKAST:Gastrointestinal disorder:0.84), (ZILEUTON:Gastrointestinal disorder:0.36), (AMISULPRIDE:Gastrointestinal disorder therapy:0.04), (BISACODYL OR DOCUSATE SODIUM:Gastrointestinal disorder therapy:0.32), (BUPROPION:Gastrointestinal disorder therapy:0.09), (CALCIUM CITRATE:Gastrointestinal disorder therapy:0.11), (CALCIUMVITAMIN D:Gastrointestinal disorder therapy:0.37), (CANDESARTAN CILEXETILHYDROCHLOROTHIAZIDE:Gastrointestinal disorder therapy:0.78), (CEPHALEXIN:Gastrointestinal disorder therapy:1.07), (CILASTATIN SODIUMIMIPENEM:Gastrointestinal disorder therapy:0.44), (CLONIDINE:Gastrointestinal disorder therapy:0.37), (CORTISONEHYDROCORTISONE:Gastrointestinal disorder therapy:0.17), (DABIGATRAN:Gastrointestinal disorder therapy:0.07), (DASATINIB:Gastrointestinal disorder therapy:0.46), (DIVALPROEX SODIUM:Gastrointestinal disorder therapy:0.4), (DUTASTERIDE:Gastrointestinal disorder therapy:0.35), (ESZOPICLONE:Gastrointestinal disorder therapy:0.66), (EVEROLIMUS:Gastrointestinal disorder therapy:0.41), (EVOLOCUMAB:Gastrointestinal disorder therapy:0.13), (FERROUS SULFATE:Gastrointestinal disorder therapy:0.87), (FLECAINIDE:Gastrointestinal disorder therapy:0.45), (FLUTICASONE:Gastrointestinal disorder therapy:0.13), (GLYCOPYRRONIUMINDACATEROL:Gastrointestinal disorder therapy:0.05), (HEPARIN CALCIUM:Gastrointestinal disorder therapy:0.03), (HYDROXYCHLOROQUINE DIPHOSPHATE:Gastrointestinal disorder therapy:0.07), (HYDROXYCHLOROQUINE SULFATE:Gastrointestinal disorder therapy:0.21), (HYDROXYZINE PAMOATE:Gastrointestinal disorder therapy:0.15), (IDELALISIB:Gastrointestinal disorder therapy:0.09), (ILOPROST:Gastrointestinal disorder therapy:0.15), (IPRATROPIUM:Gastrointestinal disorder therapy:0.13), (LABETALOLLABETALOL HYDROCHLORIDE:Gastrointestinal disorder therapy:0.34), (LATANOPROST:Gastrointestinal disorder therapy:0.14), (LEDIPASVIRSOFOSBUVIR:Gastrointestinal disorder therapy:0.38), (LOVASTATIN:Gastrointestinal disorder therapy:0.51), (MOXIFLOXACIN:Gastrointestinal disorder therapy:0.14), (NIACIN:Gastrointestinal disorder therapy:0.03), (OMEGA-3-ACID ETHYL ESTERS:Gastrointestinal disorder therapy:0.03), (PANTOPRAZOLE MAGNESIUM:Gastrointestinal disorder therapy:0.31), (PAROXETINE:Gastrointestinal disorder therapy:0.43), (PENICILLIN:Gastrointestinal disorder therapy:0.09), (PRAMIPEXOLE DIHYDROCHLORIDE:Gastrointestinal disorder therapy:0.09), (PROCHLORPERAZINE MALEATE:Gastrointestinal disorder therapy:0.01), (RANOLAZINE:Gastrointestinal disorder therapy:0.65), (SACUBITRILVALSARTAN:Gastrointestinal disorder therapy:0.73), (TERIFLUNOMIDE:Gastrointestinal disorder therapy:0.01), (TOLTERODINE TARTRATE:Gastrointestinal disorder therapy:0.02), (TRIAMCINOLONE ACETONIDE:Gastrointestinal disorder therapy:0.26), (VALGANCICLOVIR HYDROCHLORIDE:Gastrointestinal disorder therapy:0.21), (VANCOMYCIN HYDROCHLORIDE:Gastrointestinal disorder therapy:0.2), (VERAPAMIL HYDROCHLORIDE:Gastrointestinal disorder therapy:0.85), (VINCRISTINE SULFATE:Gastrointestinal disorder therapy:0.37), (VITAMIN B:Gastrointestinal disorder therapy:0.11), (ANASTROZOLE:Gastrointestinal haemorrhage:0.27), (BUPROPION HYDROCHLORIDE:Gastrointestinal haemorrhage:0.43), (CALCIUM CARBONATE:Gastrointestinal haemorrhage:0.31), (CANDESARTAN CILEXETILHYDROCHLOROTHIAZIDE:Gastrointestinal haemorrhage:0.41), (CARFILZOMIB:Gastrointestinal haemorrhage:0.17), (CERTOLIZUMAB PEGOL:Gastrointestinal haemorrhage:0.45), (CHOLESTYRAMINE:Gastrointestinal haemorrhage:0.14), (CLOTRIMAZOLE:Gastrointestinal haemorrhage:0.19), (CLOZAPINE:Gastrointestinal haemorrhage:0.21), (CORTISONEHYDROCORTISONE:Gastrointestinal haemorrhage:0.02), (DASATINIB:Gastrointestinal haemorrhage:0.57), (DEXLANSOPRAZOLE:Gastrointestinal haemorrhage:0.58), (DIVALPROEX SODIUM:Gastrointestinal haemorrhage:0.48), (DOXAZOSINDOXAZOSIN MESYLATE:Gastrointestinal haemorrhage:0.06), (ENALAPRIL MALEATE:Gastrointestinal haemorrhage:0.24), (ESZOPICLONE:Gastrointestinal haemorrhage:0.32), (EVEROLIMUS:Gastrointestinal haemorrhage:0.65), (EVOLOCUMAB:Gastrointestinal haemorrhage:0.5), (EZETIMIBE:Gastrointestinal haemorrhage:0.21), (FENOFIBRATE:Gastrointestinal haemorrhage:0.26), (FLECAINIDE:Gastrointestinal haemorrhage:0.33), (FLUTICASONE:Gastrointestinal haemorrhage:0.03), (ILOPROST:Gastrointestinal haemorrhage:0.26), (IMATINIB MESYLATE:Gastrointestinal haemorrhage:0.15), (IPRATROPIUM:Gastrointestinal haemorrhage:0.12), (LERCANIDIPINE HYDROCHLORIDE:Gastrointestinal haemorrhage:0.04), (LITHIUM CARBONATE:Gastrointestinal haemorrhage:0.09), (NALOXONE HYDROCHLORIDEOXYCODONE HYDROCHLORIDE:Gastrointestinal haemorrhage:0.17), (NEBIVOLOL:Gastrointestinal haemorrhage:1.3), (NEVIRAPINE:Gastrointestinal haemorrhage:0.22), (NILOTINIB:Gastrointestinal haemorrhage:0.87), (NITROFURANTOIN:Gastrointestinal haemorrhage:0.12), (PONATINIB:Gastrointestinal haemorrhage:0.36), (PRIMIDONE:Gastrointestinal haemorrhage:0), (PROMETHAZINEPROMETHAZINE HYDROCHLORIDE:Gastrointestinal haemorrhage:0.17), (RANOLAZINE:Gastrointestinal haemorrhage:0.63), (SECUKINUMAB:Gastrointestinal haemorrhage:0.01), (TINZAPARIN SODIUM:Gastrointestinal haemorrhage:0.01), (TOFACITINIB CITRATE:Gastrointestinal haemorrhage:0.12), (TRIAMCINOLONE ACETONIDE:Gastrointestinal haemorrhage:0.27), (VALGANCICLOVIR HYDROCHLORIDE:Gastrointestinal haemorrhage:0.19), (ZONISAMIDE:Gastrointestinal haemorrhage:0.05), (.ALPHA.1-PROTEINASE INHIBITOR HUMAN:Gastrointestinal motility disorder:0.08), (ACETAMINOPHENBUTALBITALCAFFEINE:Gastrointestinal motility disorder:0.13), (AMLODIPINE BESYLATEHYDROCHLOROTHIAZIDEOLMESARTAN MEDOXOMIL:Gastrointestinal motility disorder:0.01), (ANASTROZOLE:Gastrointestinal motility disorder:0.12),(ARMODAFINIL:Gastrointestinal motility disorder:0.11), (BUPROPION:Gastrointestinal motility disorder:0.12), (CALCIUM CARBONATECHOLECALCIFEROL:Gastrointestinal motility disorder:0.89), (CANDESARTAN:Gastrointestinal motility disorder:0.19), (CARFILZOMIB:Gastrointestinal motility disorder:0.05), (CEPHALEXIN:Gastrointestinal motility disorder:0.53), (CERTOLIZUMAB PEGOL:Gastrointestinal motility disorder:0.16), (CIPROFLOXACIN HYDROCHLORIDE:Gastrointestinal motility disorder:0.69), (CLINDAMYCINCLINDAMYCIN PHOSPHATE:Gastrointestinal motility disorder:0.95), (COLCHICINE:Gastrointestinal motility disorder:0.5), (CORTISONEHYDROCORTISONE:Gastrointestinal motility disorder:0.02), (CYCLOBENZAPRINE:Gastrointestinal motility disorder:0.36), (DASATINIB:Gastrointestinal motility disorder:0.22), (DOXAZOSIN MESYLATE:Gastrointestinal motility disorder:0.05), (ELETRIPTAN HYDROBROMIDE:Gastrointestinal motility disorder:0.05), (ENALAPRIL MALEATE:Gastrointestinal motility disorder:0.22), (ENOXAPARIN:Gastrointestinal motility disorder:0.84), (EPINEPHRINE:Gastrointestinal motility disorder:0.26), (ERGOCALCIFEROL:Gastrointestinal motility disorder:0.74), (EVEROLIMUS:Gastrointestinal motility disorder:0.57), (EVOLOCUMAB:Gastrointestinal motility disorder:0.22), (FLECAINIDE:Gastrointestinal motility disorder:0.17), (HYDROCHLOROTHIAZIDELISINOPRIL:Gastrointestinal motility disorder:0.15), (HYDROXYZINE PAMOATE:Gastrointestinal motility disorder:0.12), (ILOPROST:Gastrointestinal motility disorder:0.2), (ISOSORBIDE:Gastrointestinal motility disorder:0.13), (LABETALOLLABETALOL HYDROCHLORIDE:Gastrointestinal motility disorder:0.19), (LERCANIDIPINE:Gastrointestinal motility disorder:0.21), (LITHIUM CARBONATE:Gastrointestinal motility disorder:0.32), (METAXALONE:Gastrointestinal motility disorder:0.48), (NAPROXEN SODIUM:Gastrointestinal motility disorder:0.12), (NEBIVOLOL:Gastrointestinal motility disorder:0.25), (NILOTINIB:Gastrointestinal motility disorder:0.26), (OMEGA-3-ACID ETHYL ESTERS:Gastrointestinal motility disorder:0.07), (PANTOPRAZOLE MAGNESIUM:Gastrointestinal motility disorder:0.58), (PAROXETINE:Gastrointestinal motility disorder:0.05), (PENICILLIN:Gastrointestinal motility disorder:0.01), (PHENYTOIN:Gastrointestinal motility disorder:0.41), (PRAMIPEXOLE:Gastrointestinal motility disorder:0.38), (PRAMIPEXOLE DIHYDROCHLORIDE:Gastrointestinal motility disorder:0.49), (PRIMIDONE:Gastrointestinal motility disorder:0.38), (QUETIAPINE FUMARATE:Gastrointestinal motility disorder:0.25), (RANOLAZINE:Gastrointestinal motility disorder:0.56), (ROFLUMILAST:Gastrointestinal motility disorder:0.07), (SACUBITRILVALSARTAN:Gastrointestinal motility disorder:0.35), (SULFAMETHOXAZOLE:Gastrointestinal motility disorder:0.05), (SUMATRIPTANSUMATRIPTAN SUCCINATE:Gastrointestinal motility disorder:0.07), (TRIAMCINOLONE ACETONIDE:Gastrointestinal motility disorder:0.33), (VALGANCICLOVIR HYDROCHLORIDE:Gastrointestinal motility disorder:0.12), (VALSARTAN:Gastrointestinal motility disorder:1.17), (VARENICLINE TARTRATE:Gastrointestinal motility disorder:0.44), (VITAMIN B:Gastrointestinal motility disorder:0.32), (WARFARIN:Gastrointestinal motility disorder:1.4), (AMIODARONE:Gastrointestinal pain:0), (ANASTROZOLE:Gastrointestinal pain:0.28), (BISOPROLOL FUMARATE:Gastrointestinal pain:0.03), (BUSPIRONE HYDROCHLORIDE:Gastrointestinal pain:0.36), (CANDESARTAN CILEXETIL:Gastrointestinal pain:0.11), (CERTOLIZUMAB PEGOL:Gastrointestinal pain:1.1), (CODEINE:Gastrointestinal pain:0.05), (COLCHICINE:Gastrointestinal pain:0.12), (DIGOXIN:Gastrointestinal pain:0.51), (DIPHENHYDRAMINE:Gastrointestinal pain:0.38), (DIVALPROEX SODIUM:Gastrointestinal pain:0.8), (ERGOCALCIFEROL:Gastrointestinal pain:0.16), (EVEROLIMUS:Gastrointestinal pain:0.02), (EVOLOCUMAB:Gastrointestinal pain:0.55), (FERROUS SULFATEFERROUS SULFATE, DRIED:Gastrointestinal pain:0.43), (IBRUTINIB:Gastrointestinal pain:0.55), (METAXALONE:Gastrointestinal pain:0.5), (PAROXETINE:Gastrointestinal pain:0.15), (PRAMIPEXOLE DIHYDROCHLORIDE:Gastrointestinal pain:0.33), (PRAVASTATIN SODIUM:Gastrointestinal pain:0.38), (PRIMIDONE:Gastrointestinal pain:0), (RANOLAZINE:Gastrointestinal pain:0.02), (SACUBITRILVALSARTAN:Gastrointestinal pain:0.13), (TERIFLUNOMIDE:Gastrointestinal pain:0.52), (VARENICLINE TARTRATE:Gastrointestinal pain:0.79), (VITAMIN B:Gastrointestinal pain:0.15), (ACARBOSE:Gastrointestinal stromal tumour:0.02), (BUSPIRONE HYDROCHLORIDE:Gastrointestinal stromal tumour:0.22), (CALCIUM ACETATE:Gastrointestinal stromal tumour:0.06), (CERTOLIZUMAB PEGOL:Gastrointestinal stromal tumour:0.49), (CLINDAMYCINCLINDAMYCIN PHOSPHATE:Gastrointestinal stromal tumour:0.51), (COLCHICINE:Gastrointestinal stromal tumour:0.66), (DESLORATADINE:Gastrointestinal stromal tumour:0.12), (DOFETILIDE:Gastrointestinal stromal tumour:0.03), (EVOLOCUMAB:Gastrointestinal stromal tumour:0.54), (FULVESTRANT:Gastrointestinal stromal tumour:0.2), (HYDROMORPHONE HYDROCHLORIDE:Gastrointestinal stromal tumour:0.05), (HYDROXYUREA:Gastrointestinal stromal tumour:0.16), (IBRUTINIB:Gastrointestinal stromal tumour:1.11), (INSULIN HUMAN:Gastrointestinal stromal tumour:0.44), (LEDIPASVIRSOFOSBUVIR:Gastrointestinal stromal tumour:0.24), (MOXIFLOXACIN HYDROCHLORIDE:Gastrointestinal stromal tumour:0.03), (NALOXONE HYDROCHLORIDEOXYCODONE HYDROCHLORIDE:Gastrointestinal stromal tumour:0.29), (NAPROXEN:Gastrointestinal stromal tumour:1.21), (NEBIVOLOL:Gastrointestinal stromal tumour:0.81), (NIACIN:Gastrointestinal stromal tumour:0.22), (NITROFURANTOIN:Gastrointestinal stromal tumour:0.41), (OMEGA-3-ACID ETHYL ESTERS:Gastrointestinal stromal tumour:0.34), (OXCARBAZEPINE:Gastrointestinal stromal tumour:0.6), (PHENOBARBITAL:Gastrointestinal stromal tumour:0.16), (PRAMIPEXOLE:Gastrointestinal stromal tumour:0.18), (PRAMIPEXOLE DIHYDROCHLORIDE:Gastrointestinal stromal tumour:0.19), (QUETIAPINE:Gastrointestinal stromal tumour:0.07), (RABEPRAZOLE:Gastrointestinal stromal tumour:0.09), (RANOLAZINE:Gastrointestinal stromal tumour:0.62), (RISEDRONATE SODIUM:Gastrointestinal stromal tumour:0.26), (SACUBITRILVALSARTAN:Gastrointestinal stromal tumour:0.63), (SODIUM OXYBATE:Gastrointestinal stromal tumour:0.31), (TETRACYCLINE:Gastrointestinal stromal tumour:0.05), (TRIAMCINOLONE ACETONIDE:Gastrointestinal stromal tumour:0.26), (VARENICLINE TARTRATE:Gastrointestinal stromal tumour:0.01), (VERAPAMIL HYDROCHLORIDE:Gastrointestinal stromal tumour:1.2), (.ALPHA.-LIPOIC ACID, (+/−)-:Gastrooesophageal reflux disease:0.12), (.ALPHA.-TOCOPHEROL ACETATE, D-ASCORBIC ACIDCALCIUM PANTOTHENATEFOLIC ACIDMANGANESENIACINAMIDEPYRIDOXINERIBOFLAVINTHIAMINEVITAMIN AZINC OXIDE:Gastrooesophageal reflux disease:0.26), (.BETA.-ACETYLDIGOXIN:Gastrooesophageal reflux disease:0.36), (.BETA.-HYDROXYISOVALERIC ACIDARGININEGLUTAMINE:Gastrooesophageal reflux disease:0.99), (ACAMPROSATE:Gastrooesophageal reflux disease:0.85), (ACETAMINOPHEN AND OR DEXTROMETHORPHAN AND OR DIPHENHYDRAMINE AND OR GUAIFENESIN AND OR PHENYLEPHRINE AND OR PSEUDOEPHEDRINE:Gastrooesophageal reflux disease:0.02), (ACETAMINOPHENAPRONALIDECAFFEINEPROPYPHENAZONE:Gastro oesophageal reflux disease:1.6), (ACETAMINOPHENASPIRINSALICYLAMIDE:Gastrooesophageal reflux disease:0.12), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Gastrooesophageal reflux disease:0.7), (ACETAMINOPHENCAFFEINECARISOPRODOLDICLOFENAC:Gastrooesop hageal reflux disease:1), (ACETAMINOPHENCAFFEINECARISOPRODOLDICLOFENAC SODIUM:Gastrooesophageal reflux disease:0.61), (ACETAMINOPHENCAFFEINECODEINE PHOSPHATE:Gastrooesophageal reflux disease:0.21), (ACETAMINOPHENDEXTROMETHORPHAN HYDROBROMIDEPHENYLEPHRINE HYDROCHLORIDE:Gastrooesophageal reflux disease:0.26), (ACETAMINOPHENMETHOCARBAMOL:Gastrooesophageal reflux disease:1.76), (ACETAMINOPHENPHENYLEPHRINE:Gastrooesophageal reflux disease:0.1), (ACETAMINOPHENPHENYLEPHRINE HYDROCHLORIDE:Gastrooesophageal reflux disease:0.31), (ACETOHYDROXAMIC ACID:Gastrooesophageal reflux disease:0.34), (ACETYLCYSTEINEBENZALKONIUM CHLORIDETUAMINOHEPTANE SULFATE:Gastrooesophageal reflux disease:0.59), (ACLARUBICIN HYDROCHLORIDE:Gastrooesophageal reflux disease:0.25), (ACLIDINIUM BROMIDEFORMOTEROL FUMARATE:Gastrooesophageal reflux disease:0.65), (ALBUTEROLGUAIFENESIN:Gastrooesophageal reflux disease:1.27), (ALCOHOLALUMINUM CHLORIDE:Gastrooesophageal reflux disease:0.72), (ALDESLEUKIN:Gastrooesophageal reflux disease:0.45), (ALDIOXA:Gastrooesophageal reflux disease:0.39), (ALFALFA:Gastrooesophageal reflux disease:0.86), (ALISKIRENHYDROCHLOROTHIAZIDE:Gastrooesophageal reflux disease:0.91), (ALLANTOINBENZOCAINECAMPHOR (NATURAL)DIMETHICONEMENTHOL:Gastrooesophageal reflux disease:0.65), (ALPHA-TOCOPHEROLASCORBIC ACIDCHOLECALCIFEROLCOPPERCYANOCOBALAMINFOLIC ACIDLUTEINOMEGA-3 FATTY ACIDSPYRIDOXINE HYDROCHLORIDETAURINEVITAMIN A PALMITATEZEAXANTHINZINC PICOLINATE:Gastrooesophageal reflux disease:0.22), (ALUMINUM CHLORIDE:Gastrooesophageal reflux disease:0.55), (ALUMINUM SILICATE:Gastrooesophageal reflux disease:1.25), (AMANTADINE SULFATE:Gastrooesophageal reflux disease:0.29), (AMINO ACIDSVITAMINS:Gastrooesophageal reflux disease:0.54), (AMINOBENZOIC ACIDCALCIUM PANTOTHENATECYSTINEKERATINTHIAMINE MONONITRATE:Gastrooesophageal reflux disease:0.92), (AMINOPHYLLINEQUININE SULFATE:Gastrooesophageal reflux disease:0.3), (AMITRIPTYLINEPERPHENAZINE:Gastrooesophageal reflux disease:0.57), (AMLODIPINE BESYLATEATORVASTATIN CALCIUM TRIHYDRATE:Gastrooesophageal reflux disease:0.12), (AMLODIPINE MALEATE:Gastrooesophageal reflux disease:0.62), (AMLODIPINEATORVASTATIN:Gastrooesophageal reflux disease:0.18), (AMOXICILLIN SODIUM:Gastrooesophageal reflux disease:0.66), (AMPHETAMINE ASPARTATE MONOHYDRATEAMPHETAMINE SULFATEDEXTROAMPHETAMINE SACCHARATEDEXTROAMPHETAMINE SULFATE:Gastrooesophageal reflux disease:0), (AMPHETAMINE ASPARTATEDEXTROAMPHETAMINE SACCHARATE:Gastrooesophageal reflux disease:0.29), (AMPHETAMINE SULFATE:Gastrooesophageal reflux disease:0.75), (ANGIOTENSIN II:Gastrooesophageal reflux disease:0.02), (ANTIHEMOPHILIC FACTOR, HUMAN RECOMBINANT RESIDUES 743-1636 DELETED:Gastrooesophageal reflux disease:0.33), (ARCTOSTAPHYLOS UVA-URSI LEAFURTICA DIOICA:Gastrooesophageal reflux disease:0.85), (ARIPIPRAZOLE LAUROXIL:Gastrooesophageal reflux disease:1.35), (ASCORBIC ACIDBIOTINCALCIUM PANTOTHENATECYANOCOBALAMINFOLIC ACIDNIACINAMIDEPYRIDOXINE HYDROCHLORIDERIBOFLAVINTHIAMINE HYDROCHLORIDE:Gastrooesophageal reflux disease:0.38), (ASCORBIC ACIDCHONDROITIN SULFATE (BOVINE)GLUCOSAMINE SULFATEMANGANESE:Gastrooesophageal reflux disease:0.44), (ASCORBIC ACIDFERROUS SULFATEFOLIC ACIDPYRIDOXINE HYDROCHLORIDERIBOFLAVINTHIAMINE MONONITRATE:Gastrooesophageal reflux disease:0.9), (ASCORBIC ACIDVITAMIN B COMPLEX:Gastrooesophageal reflux disease:0.36), (ASPARTAME:Gastrooesophageal reflux disease:0.08), (ASPIRINCAFFEINESALICYLAMIDE:Gastrooesophageal reflux disease:1.47), (ASPIRINMAGNESIUM HYDROXIDE:Gastrooesophageal reflux disease:0.22), (AZASETRON HYDROCHLORIDE:Gastrooesophageal reflux disease:0.31), (AZITHROMYCIN:Gastrooesophageal reflux disease:0.63), (BACILLUS CALMETTE-GUERIN SUBSTRAIN CONNAUGHT LIVE ANTIGEN:Gastrooesophageal reflux disease:0.04), (BACITRACIN ZINC:Gastrooesophageal reflux disease:0.49), (BACITRACINNEOMYCIN SULFATE:Gastrooesophageal reflux disease:0.78), (BACITRACINNEOMYCIN SULFATEPOLYMYXIN B SULFATE:Gastrooesophageal reflux disease:0.29), (BACLOFENCYCLOBENZAPRINEDICLOFENACGABAPENTINTETRAC AINE:Gastrooesophageal reflux disease:0.63), (BALSAM PERUBISMUTH HYDROXIDEBISMUTH SUBGALLATEBORIC ACIDZINC OXIDE:Gastrooesophageal reflux disease:0.36), (BARNIDIPINE:Gastrooesophageal reflux disease:0.37), (BENIDIPINE:Gastrooesophageal reflux disease:1.43), (BENPROPERINE PHOSPHATE:Gastrooesophageal reflux disease:0.62), (BENRALIZUMAB:Gastrooesophageal reflux disease:0.09), (BENZALKONIUM CHLORIDE:Gastrooesophageal reflux disease:0.24), (BENZETHONIUM CHLORIDE:Gastrooesophageal reflux disease:0.14), (BENZOCAINERESORCINOL:Gastrooesophageal reflux disease:0.61), (BENZOIC ACIDHYOSCYAMINE SULFATEMETHENAMINEMETHYLENE BLUEPHENYL SALICYLATE:Gastrooesophageal reflux disease:0.91), (BEPRIDIL HYDROCHLORIDE:Gastrooesophageal reflux disease:0.33), (BERBERINEGERANIUM:Gastrooesophageal reflux disease:0.38), (BETAINE:Gastrooesophageal reflux disease:0.45), (BETAMETHASONE ACETATEBETAMETHASONE SODIUM PHOSPHATE:Gastrooesophageal reflux disease:0.1), (BETAMETHASONE DIPROPIONATECALCIPOTRIENE HYDRATE:Gastrooesophageal reflux disease:1.49), (BETAMETHASONE DIPROPIONATECLOTRIMAZOLEGENTAMICIN SULFATE:Gastrooesophageal reflux disease:0.21), (BETAMETHASONE DIPROPIONATEGENTAMICIN SULFATE:Gastrooesophageal reflux disease:0.29), (BETAMIPRONPANIPENEM:Gastrooesophageal reflux disease:0.58), (BEXAROTENE:Gastrooesophageal reflux disease:0.46), (BICARBONATE IONPOLYETHYLENE GLYCOLSPOTASSIUM CATIONSODIUM CHLORIDESODIUM SULFATE ANHYDROUS:Gastrooesophageal reflux disease:0.58), (BIOTINCALCIUM PANTOTHENATECHOLINE BITARTRATECYANOCOBALAMINFOLIC ACIDINOSITOLNIACINAMIDEPYRIDOXINERIBOFLAVINTHIAMINE:Gastrooesopha geal reflux disease:0.27), (BIPERIDEN LACTATE:Gastrooesophageal reflux disease:0.18), (BISMUTH:Gastrooesophageal reflux disease:0.47), (BISMUTH SUBCITRATERANITIDINE HYDROCHLORIDESUCRALFATE:Gastrooesophageal reflux disease:0.38), (BISMUTH SUBSALICYLATEGLYCERINPROPYLENE GLYCOL:Gastrooesophageal reflux disease:0.6), (BORIC ACID:Gastrooesophageal reflux disease:0.81), (BORON:Gastrooesophageal reflux disease:0.85), (BOTULINUM TOXIN TYPE A:Gastrooesophageal reflux disease:1.94), (BROMPERIDOL:Gastrooesophageal reflux disease:0.35), (BUCOLOME:Gastrooesophageal reflux disease:0.58), (BUDESONIDEINDACATEROL MALEATE:Gastrooesophageal reflux disease:0.28), (BUFORMIN:Gastrooesophageal reflux disease:1.03), (CAFFEINEDIPYRONEORPHENADRINE:Gastrooesophageal reflux disease:1.37), (CAFFEINEISOMETHEPTENEMETHIMAZOLE:Gastrooesophageal reflux disease:0.58), (CAJUPUT OILEUCALYPTUS OILPEPPERMINT OIL:Gastrooesophageal reflux disease:1.36), (CALCIUM BICARBONATE:Gastrooesophageal reflux disease:1.33), (CALCIUM CARBONATEDIMETHICONE:Gastrooesophageal reflux disease:0.06), (CALCIUM CARBONATEERGOCALCIFEROL:Gastrooesophageal reflux disease:1.75), (CALCIUM CHLORIDEDEXTROSE MONOHYDRATEMAGNESIUM CHLORIDESODIUM CHLORIDESODIUM LACTATE:Gastrooesophageal reflux disease:0.2), (CALCIUM CHLORIDEPOTASSIUM CHLORIDESODIUM CHLORIDESODIUM LACTATESORBITOL:Gastrooesophageal reflux disease:0.11), (CALCIUM PANTOTHENATENIACINAMIDEPYRIDOXINE HYDROCHLORIDERIBOFLAVINTHIAMINE HYDROCHLORIDE:Gastrooesophageal reflux disease:0.1), (CALCIUMERGOCALCIFEROL:Gastrooesophageal reflux disease:0.58), (CAMPHOR (SYNTHETIC)CAPSICUM OLEORESINMENTHOLMETHYL SALICYLATE:Gastrooesophageal reflux disease:0.16), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:Gastrooesophageal reflux disease:0.54), (CAPSICUM:Gastrooesophageal reflux disease:0.82), (CARBAZOCHROME:Gastrooesophageal reflux disease:0.18), (CARPRONIUM CHLORIDE:Gastrooesophageal reflux disease:0.73), (CAT'S CLAW:Gastrooesophageal reflux disease:0.31), (CEFCAPENE PIVOXIL HYDROCHLORIDE:Gastrooesophageal reflux disease:1.59), (CEFOXITINCEFOXITIN SODIUM:Gastrooesophageal reflux disease:0.6), (CEFOZOPRAN HYDROCHLORIDE:Gastrooesophageal reflux disease:0.57), (CEFPIROME SULFATE:Gastrooesophageal reflux disease:0.24), (CEFPROZIL:Gastrooesophageal reflux disease:1.88), (CEFTAZIDIME SODIUM:Gastrooesophageal reflux disease:1.09), (CEFTERAM PIVOXIL:Gastrooesophageal reflux disease:1.1), (CEFUROXIME SODIUM:Gastrooesophageal reflux disease:0.24), (CEPHALOSPORIN C:Gastrooesophageal reflux disease:0.09), (CERTOPARIN SODIUM:Gastrooesophageal reflux disease:1.85), (CHAMOMILE:Gastrooesophageal reflux disease:0.59), (CHIMAPHILA UMBELLATAMANGANESE CHLORIDEPOPULUS TREMULOIDESSODIUM TAUROCHOLATE:Gastrooesophageal reflux disease:0.89), (CHLOPHEDIANOL HYDROCHLORIDEDEXBROMPHENIRAMINE MALEATEPHENYLEPHRINE HYDROCHLORIDE:Gastrooesophageal reflux disease:0.38), (CHLORAMPHENICOLPREDNISOLONE:Gastrooesophageal reflux disease:0.05), (CHLORIDE ION:Gastrooesophageal reflux disease:0.06), (CHLORMADINONE ACETATEETHINYL ESTRADIOL:Gastrooesophageal reflux disease:1.3), (CHLOROQUINE PHOSPHATE:Gastrooesophageal reflux disease:0.36), (CHLORPHENIRAMINE MALEATEHYDROCODONE BITARTRATEPSEUDOEPHEDRINE HYDROCHLORIDE:Gastrooesophageal reflux disease:0.32), (CHLORPHENIRAMINE MALEATEPHENYLEPHRINE HYDROCHLORIDE:Gastrooesophageal reflux disease:0.25), (CHLORPROMAZINE HYDROCHLORIDEPHENOBARBITALPROMETHAZINE:Gastrooesophageal reflux disease:0.33), (CHLORPROMAZINEPROMETHAZINE:Gastrooesophageal reflux disease:0.39), (CHLORPROTHIXENE:Gastrooesophageal reflux disease:1.08), (CHOLECALCIFEROLGENISTEINZINC GLYCINATE:Gastrooesophageal reflux disease:0.86), (CHOLINE:Gastrooesophageal reflux disease:0.17), (CHOLINE BITARTRATE:Gastrooesophageal reflux disease:0.19), (CHOLINETRAZODONE HYDROCHLORIDE:Gastrooesophageal reflux disease:0.58), (CICLOPIROX OLAMINE:Gastrooesophageal reflux disease:1.69), (CINNAMON OIL:Gastrooesophageal reflux disease:0.01), (CIPROFLOXACINDEXAMETHASONE:Gastrooesophageal reflux disease:0.25), (CITICOLINE:Gastrooesophageal reflux disease:0.41), (CITRIC ACID MONOHYDRATETRISODIUM CITRATE DIHYDRATE:Gastrooesophageal reflux disease:0.47), (CLOBETASONE BUTYRATENYSTATINOXYTETRACYCLINE CALCIUM:Gastrooesophageal reflux disease:0.06), (CLOMETHIAZOLE:Gastrooesophageal reflux disease:0.47), (CLONIXINCYCLOBENZAPRINE:Gastrooesophageal reflux disease:0.3), (CLOSTRIDIUM TETANICLOSTRIDIUM TETANI TOXOID ANTIGEN (FORMALDEHYDE INACTIVATED)CORYNEBACTERIUM DIPHTHERIAECORYNEBACTERIUM DIPHTHERIAE TOXOID ANTIGEN (FORMALDEHYDE INACTIVATED):Gastrooesophageal reflux disease:0.26), (COAGULATION FACTOR IX HUMANCOAGULATION FACTOR VII HUMANCOAGULATION FACTOR X HUMANPROTEIN CPROTEIN S HUMANPROTHROMBIN:Gastrooesophageal reflux disease:0.22), (COAGULATION FACTOR IX RECOMBINANT HUMANISOPROPYL ALCOHOL:Gastrooesophageal reflux disease:0.48), (COBALAMIN:Gastrooesophageal reflux disease:0.68), (COBAMAMIDE:Gastrooesophageal reflux disease:0), (COBICISTATDARUNAVIR ETHANOLATE:Gastrooesophageal reflux disease:0.42), (CODEINE PHOSPHATEPHENYLEPHRINE HYDROCHLORIDEPROMETHAZINE HYDROCHLORIDE:Gastrooesophageal reflux disease:0.53), (CODEINE PHOSPHATEPROMETHAZINE:Gastrooesophageal reflux disease:1.17), (COLCHICINEDICYCLOMINE HYDROCHLORIDE:Gastrooesophageal reflux disease:0.94), (CREATINE MONOHYDRATE:Gastrooesophageal reflux disease:0.92), (CREATININE:Gastrooesophageal reflux disease:0.39), (CROFELEMER:Gastrooesophageal reflux disease:0.48), (CYANAMIDE:Gastrooesophageal reflux disease:0.16), (CYANOCOBALAMINDICLOFENAC SODIUMPYRIDOXINE HYDROCHLORIDETHIAMINE MONONITRATE:Gastrooesophageal reflux disease:0.35), (CYANOCOBALAMINPYRIDOXINE HYDROCHLORIDETHIAMINE DISULFIDE:Gastrooesophageal reflux disease:0.91), (CYPROTERONE:Gastrooesophageal reflux disease:0.89), (CYSTEAMINE BITARTRATE:Gastrooesophageal reflux disease:0.34), (CYSTINE:Gastrooesophageal reflux disease:0.45), (CYTARABINE OCFOSFATE:Gastrooesophageal reflux disease:0.09), (DARUNAVIRRITONAVIR:Gastrooesophageal reflux disease:0.35), (DASABUVIR SODIUM MONOHYDRATE:Gastrooesophageal reflux disease:0.16), (DAUNORUBICIN HYDROCHLORIDE:Gastrooesophageal reflux disease:0.69), (DEFIBROTIDE (BOVINE):Gastrooesophageal reflux disease:0.29), (DENOPAMINE:Gastrooesophageal reflux disease:0.1), (DEUTETRABENAZINE:Gastrooesophageal reflux disease:0.31), (DEXIBUPROFEN:Gastrooesophageal reflux disease:0.63), (DEXTRAN SULFATE SODIUM SALT:Gastrooesophageal reflux disease:0.53), (DEXTROMETHORPHANDEXTROMETHORPHAN HYDROBROMIDEGUAIFENESIN:Gastrooesophageal reflux disease:0.8), (DIACEREIN:Gastrooesophageal reflux disease:0.58), (DIASTASE:Gastrooesophageal reflux disease:0.53), (DICHLORPHENAMIDE:Gastrooesophageal reflux disease:0.67), (DICLOFENAC DIETHYLAMINE:Gastrooesophageal reflux disease:0.61), (DIENOGEST:Gastrooesophageal reflux disease:1.36), (DIETARY SUPPLEMENTFLAVOCOXID:Gastrooesophageal reflux disease:0.64), (DIETARY SUPPLEMENTRESVERATROL:Gastrooesophageal reflux disease:0.81), (DIFLUCORTOLONE VALERATELIDOCAINE:Gastrooesophageal reflux disease:0.87), (DIHYDRALAZINE SULFATE:Gastrooesophageal reflux disease:0.14), (DIHYDROERGOCRISTINE MESYLATE:Gastrooesophageal reflux disease:0.11), (DIMEMORFAN PHOSPHATE:Gastrooesophageal reflux disease:1.13), (DIMETHYL SULFOXIDE:Gastrooesophageal reflux disease:1.73), (DIPYRONEPROMETHAZINE HYDROCHLORIDE:Gastrooesophageal reflux disease:2.54), (DIQUAFOSOL TETRASODIUM:Gastrooesophageal reflux disease:0.61), (DOCONEXENT:Gastrooesophageal reflux disease:0.1), (DOCONEXENTICOSAPENT:Gastrooesophageal reflux disease:0.06), (DOCUSATE SODIUMSENNOSIDE ASENNOSIDE B:Gastrooesophageal reflux disease:0.73), (DROSPIRENONE:Gastrooesophageal reflux disease:0.31), (DYCLONINE HYDROCHLORIDE:Gastrooesophageal reflux disease:0.1), (ELM:Gastrooesophageal reflux disease:0.01), (EPHEDRINE HYDROCHLORIDE:Gastrooesophageal reflux disease:0.11), (EPHEDRINE HYDROCHLORIDEGUAIFENESIN:Gastrooesophageal reflux disease:1.48), (EPRAZINONE:Gastrooesophageal reflux disease:1.32), (ERGOLOID MESYLATES:Gastrooesophageal reflux disease:1.09), (ERYTHROMYCIN LACTOBIONATE:Gastrooesophageal reflux disease:1.35), (ERYTHROMYCINSULFISOXAZOLE:Gastrooesophageal reflux disease:0.94), (ESCHERICHIA COLI:Gastrooesophageal reflux disease:0.3), (ESMOLOL HYDROCHLORIDE:Gastrooesophageal reflux disease:0.87), (ESSENTIAL OILS:Gastrooesophageal reflux disease:0.16), (ESTRADIOL BENZOATE:Gastrooesophageal reflux disease:1.79), (ESTRADIOLLEVONORGESTREL:Gastrooesophageal reflux disease:0.76), (ESTRONE:Gastrooesophageal reflux disease:0.51), (ETELCALCETIDE HYDROCHLORIDE:Gastrooesophageal reflux disease:0.03), (ETEPLIRSEN:Gastrooesophageal reflux disease:0.17), (ETHINYL ESTRADIOL:Gastrooesophageal reflux disease:0.51), (ETHINYL ESTRADIOLETHYNODIOL:Gastrooesophageal reflux disease:0.04), (ETHOSUXIMIDE:Gastrooesophageal reflux disease:0.5), (ETILEFRINE HYDROCHLORIDE:Gastrooesophageal reflux disease:0.64), (FELODIPINEMETOPROLOL SUCCINATE:Gastrooesophageal reflux disease:0.46), (FENOPROFEN:Gastrooesophageal reflux disease:0.42), (FERRIC SULFATE:Gastrooesophageal reflux disease:0), (FERRITIN:Gastrooesophageal reflux disease:1.34), (FIBRINOGEN HUMAN:Gastrooesophageal reflux disease:0.45), (FIMASARTAN:Gastrooesophageal reflux disease:0.53), (FINGOLIMOD:Gastrooesophageal reflux disease:0.65), (FLOMOXEF:Gastrooesophageal reflux disease:0.89), (FLUCYTOSINE:Gastrooesophageal reflux disease:0.32), (FLUOROURACILLEUCOVORIN CALCIUMOXALIPLATIN:Gastrooesophageal reflux disease:1.05), (FLUPHENAZINE DECANOATE:Gastrooesophageal reflux disease:0.13), (FORMALDEHYDE:Gastrooesophageal reflux disease:0.02), (FOSFOMYCIN CALCIUM:Gastrooesophageal reflux disease:1.35), (FUSIDATE SODIUM:Gastrooesophageal reflux disease:0.37), (GENISTEIN:Gastrooesophageal reflux disease:0.34), (GENTIAN VIOLET:Gastrooesophageal reflux disease:0.87), (GINSENG EXTRACT:Gastrooesophageal reflux disease:0.16), (GLIMEPIRIDEMETFORMIN:Gastrooesophageal reflux disease:1.08), (GLIMEPIRIDEPIOGLITAZONE HYDROCHLORIDE:Gastrooesophageal reflux disease:0.13), (GLIMEPIRIDEROSIGLITAZONE MALEATE:Gastrooesophageal reflux disease:0.62), (GLYCERINMINERAL OILPARAFFIN:Gastrooesophageal reflux disease:0.02), (GLYCEROL PHENYLBUTYRATE:Gastrooesophageal reflux disease:0), (GOLD SODIUM THIOMALATE:Gastrooesophageal reflux disease:0.05), (GRAPEFRUIT SEED EXTRACT:Gastrooesophageal reflux disease:0.04), (HALCINONIDE:Gastrooesophageal reflux disease:0.85), (HARPAGOPHYTUM PROCUMBENS ROOT:Gastrooesophageal reflux disease:0.06), (HEMP:Gastrooesophageal reflux disease:0.38), (HERBALSHOMEOPATHICSGOLDENSEAL:Gastrooesophageal reflux disease:0.58), (HERBALSVITIS VINIFERA SEED:Gastrooesophageal reflux disease:0.81), (HUMAN COLOSTRUM:Gastrooesophageal reflux disease:0.37), (HYDROCHLOROTHIAZIDEMETOPROLOL:Gastrooesophageal reflux disease:0.17), (HYDROCHLOROTHIAZIDEMETOPROLOL SUCCINATE:Gastrooesophageal reflux disease:1.37), (HYDROCHLOROTHIAZIDEPROPRANOLOL HYDROCHLORIDE:Gastrooesophageal reflux disease:0.07), (HYDROCORTISONE HEMISUCCINATE:Gastrooesophageal reflux disease:0.06), (HYDROCORTISONE PROBUTATE:Gastrooesophageal reflux disease:1.03), (HYDROCORTISONEMICONAZOLE:Gastrooesophageal reflux disease:0.23), (HYDROQUINONE:Gastrooesophageal reflux disease:0.75), (HYDROXOCOBALAMIN ACETATE:Gastrooesophageal reflux disease:1.03), (HYDROXOCOBALAMINPYRIDOXINE HYDROCHLORIDETHIAMINE HYDROCHLORIDE:Gastrooesophageal reflux disease:0.41), (HYDROXYPROPYL CELLULOSE:Gastrooesophageal reflux disease:1.27), (IDURSULFASE:Gastrooesophageal reflux disease:0.23), (INCOBOTULINUMTOXINA:Gastrooesophageal reflux disease:0.12), (INSULIN DEGLUDECLIRAGLUTIDE:Gastrooesophageal reflux disease:0.12), (INSULIN PORKINSULIN PURIFIED PORK:Gastrooesophageal reflux disease:1.66), (INTERFERON ALFACON-1:Gastrooesophageal reflux disease:0.02), (INTERFERON GAMMA-1B:Gastrooesophageal reflux disease:2.23), (IRON POLYMALTOSE:Gastrooesophageal reflux disease:1.62), (ISAVUCONAZOLE:Gastrooesophageal reflux disease:0.48), (ISOCARBOXAZID:Gastrooesophageal reflux disease:0.1), (ISOFLURANE:Gastrooesophageal reflux disease:0.3), (IVY EXTRACT:Gastrooesophageal reflux disease:0.1), (IXAZOMIB CITRATE:Gastrooesophageal reflux disease:0.43), (LANDIOLOL HYDROCHLORIDE:Gastrooesophageal reflux disease:0.86), (LATANOPROSTTIMOLOL:Gastrooesophageal reflux disease:1.96), (LETROZOLERIBOCICLIB:Gastrooesophageal reflux disease:0.49), (LEVAMLODIPINE:Gastrooesophageal reflux disease:0.43), (LEVAMLODIPINE BESILATE:Gastrooesophageal reflux disease:0.03), (LEVAMLODIPINE BESYLATE:Gastrooesophageal reflux disease:0.29), (LEVOMEFOLATE CALCIUMMETHYLCOBALAMINPYRIDOXAL 5-PHOSPHATE:Gastrooesophageal reflux disease:0.4), (LEVOMEFOLIC ACIDMETHYLCOBALAMINTURMERIC:Gastrooesophageal reflux disease:0.35), (LEVOMEPROMAZINE MALEATE:Gastrooesophageal reflux disease:0.63), (LEVOMILNACIPRAN:Gastrooesophageal reflux disease:0.39), (LEVORPHANOL TARTRATE:Gastrooesophageal reflux disease:0.34), (LEVOSIMENDAN:Gastrooesophageal reflux disease:0.7), (LIDOCAINETRIBENOSIDE:Gastrooesophageal reflux disease:0.21), (LINCOMYCIN:Gastrooesophageal reflux disease:0.12), (LIPASE:Gastrooesophageal reflux disease:0.45), (LIXISENATIDE:Gastrooesophageal reflux disease:0.21), (LOXAPINE:Gastrooesophageal reflux disease:0.07), (LUMACAFTOR:Gastrooesophageal reflux disease:1.43), (LUTETIUM LU-177:Gastrooesophageal reflux disease:1.67), (LYNESTRENOL:Gastrooesophageal reflux disease:0.13), (LYSINE HYDROCHLORIDE:Gastrooesophageal reflux disease:0.3), (MAGNESIUM GLUCEPTATE:Gastrooesophageal reflux disease:0.43), (MAGNESIUM PIDOLATE:Gastrooesophageal reflux disease:0.96), (MAGNESIUM STEARATE:Gastrooesophageal reflux disease:0.03), (MALTOSE:Gastrooesophageal reflux disease:0.68), (MANGANESEMANGANESE CHLORIDE:Gastrooesophageal reflux disease:0.65), (MAPROTILINE HYDROCHLORIDE:Gastrooesophageal reflux disease:1.27), (MECHLORETHAMINE:Gastrooesophageal reflux disease:0.49), (MENINGOCOCCAL GROUP B VACCINE:Gastrooesophageal reflux disease:0.03), (MEPENZOLATE BROMIDE:Gastrooesophageal reflux disease:1.29), (MESTRANOLNORETHINDRONE:Gastrooesophageal reflux disease:1.01), (METAMIZOLE MAGNESIUM:Gastrooesophageal reflux disease:0.03), (METFORMIN HYDROCHLORIDESAXAGLIPTIN:Gastrooesophageal reflux disease:1.74), (METHENOLONE ACETATE:Gastrooesophageal reflux disease:0.29), (METHIONINE:Gastrooesophageal reflux disease:0.02), (METHSUXIMIDE:Gastrooesophageal reflux disease:0.45), (METHYCLOTHIAZIDE:Gastrooesophageal reflux disease:0.17), (METHYLPREDNISOLONE OR METHYLPREDNISOLONE ACETATE:Gastrooesophageal reflux disease:0.13), (METRONIDAZOLE BENZOATE:Gastrooesophageal reflux disease:0.18), (METYROSINE:Gastrooesophageal reflux disease:0.11), (MIDOSTAURIN:Gastrooesophageal reflux disease:0.01), (MINERALSPROBIOTICS NOSVITAMINS:Gastrooesophageal reflux disease:0.12), (NADH:Gastrooesophageal reflux disease:0.4), (NALDEMEDINE:Gastrooesophageal reflux disease:0.55), (NEISSERIA MENINGITIDIS GROUP A CAPSULAR POLYSACCHARIDE DIPHTHERIA TOXOID CONJUGATE ANTIGENNEISSERIA MENINGITIDIS GROUP C CAPSULAR POLYSACCHARIDE DIPHTHERIA TOXOID CONJUGATE ANTIGENNEISSERIA MENINGITIDIS GROUP W-135 CAPSULAR POLYSACCHARIDE DIPHTHERIA TOXOID CONJUGATE ANTIGENNEISSERIA MENINGITIDIS:Gastrooesophageal reflux disease:0.22), (NEOMYCINPOLYMYXIN:Gastrooesophageal reflux disease:0.16), (NEOSTIGMINE BROMIDE:Gastrooesophageal reflux disease:0.15), (NIACINAMIDEPYRIDOXINERIBOFLAVINTHIAMINE:Gastrooesophageal reflux disease:0.56), (NIAOULI OILQUININE BENZOATETHIAMINE HYDROCHLORIDE:Gastrooesophageal reflux disease:0.22), (NIFURTOINOL:Gastrooesophageal reflux disease:0.08), (NIMODIPINE:Gastrooesophageal reflux disease:1.12), (NIPRADILOL:Gastrooesophageal reflux disease:0.04), (NUSINERSEN:Gastrooesophageal reflux disease:1.12), (OBETICHOLIC ACID:Gastrooesophageal reflux disease:0.84), (OCTINOXATEOXYBENZONE:Gastrooesophageal reflux disease:0.14), (OCTOTIAMINE:Gastrooesophageal reflux disease:0.48), (OK-432:Gastrooesophageal reflux disease:0.33), (OLODATEROL:Gastrooesophageal reflux disease:0.44), (OMBITASVIR HEMINONAHYDRATEPARITAPREVIR DIHYDRATERITONAVIR:Gastrooesophageal reflux disease:0.06), (OMEGA-6 FATTY ACIDS:Gastrooesophageal reflux disease:0.76), (OSTREA EDULIS SHELL:Gastrooesophageal reflux disease:0.34), (OXANDROLONE:Gastrooesophageal reflux disease:0.78), (OXATOMIDE:Gastrooesophageal reflux disease:0.34), (OXYPERTINE:Gastrooesophageal reflux disease:0.06), (PAMABROM:Gastrooesophageal reflux disease:0.1), (PARSLEY:Gastrooesophageal reflux disease:0.66), (PASCOPYRUM SMITHII POLLEN:Gastrooesophageal reflux disease:0.19), (PASSIFLORA INCARNATA FLOWER:Gastrooesophageal reflux disease:0.23), (PEGINTERFERON ALFA-2A OR PEGINTERFERON ALFA-2B OR PEGINTERFERON BETA-1A OR PEGINTERFERON LAMBDA-1A:Gastrooesophageal reflux disease:0.12), (PENICILLIN G BENZATHINE:Gastrooesophageal reflux disease:0.06), (PENTAZOCINE HYDROCHLORIDE:Gastrooesophageal reflux disease:0.3), (PENTOSTATIN:Gastrooesophageal reflux disease:2.03), (PENTOXYVERINE:Gastrooesophageal reflux disease:0.13), (PERICIAZINE:Gastrooesophageal reflux disease:0.11), (PEROSPIRONE:Gastrooesophageal reflux disease:0.69), (PEROSPIRONE HYDROCHLORIDE:Gastrooesophageal reflux disease:0.84), (PHENAZEPAM:Gastrooesophageal reflux disease:0.02), (PHENYLEPHRINE HYDROCHLORIDEWITCH HAZEL:Gastrooesophageal reflux disease:0.5), (PIPAMPERONE:Gastrooesophageal reflux disease:0.12), (PIRARUBICIN HYDROCHLORIDE:Gastrooesophageal reflux disease:0.11), (PIRITRAMIDE:Gastrooesophageal reflux disease:1.38), (PITOFENONE:Gastrooesophageal reflux disease:0.88), (POLYETHYLENE GLYCOL 3350\POTASSIUM CHLORIDESODIUM BICARBONATESODIUM CHLORIDESODIUM SULFATESODIUM SULFATE ANHYDROUS:Gastrooesophageal reflux disease:0.14), (POTASSIUM ACETATE:Gastrooesophageal reflux disease:0.19), (POTASSIUM BITARTRATE:Gastrooesophageal reflux disease:0.48), (POTASSIUM CHLORIDESODIUM CHLORIDESODIUM LACTATE:Gastrooesophageal reflux disease:1.33), (POTASSIUM CITRATESODIUM CITRATEZINC:Gastrooesophageal reflux disease:0.3), (PREDNISOLONE SODIUM METAZOATE:Gastrooesophageal reflux disease:0.58), (PROMAZINE:Gastrooesophageal reflux disease:0.74), (PROMAZINE HYDROCHLORIDE:Gastrooesophageal reflux disease:0.57), (PROPANTHELINE BROMIDE:Gastrooesophageal reflux disease:0.4), (PROPOLIS WAX:Gastrooesophageal reflux disease:0.03), (PSORALEN:Gastrooesophageal reflux disease:0.57), (PYRIDOXAL PHOSPHATE:Gastrooesophageal reflux disease:1.24), (QUINACRINE:Gastrooesophageal reflux disease:2.86), (QUINAGOLIDE HYDROCHLORIDE:Gastrooesophageal reflux disease:0.75), (QUINIDINE GLUCONATE:Gastrooesophageal reflux disease:0.59), (RADIUM:Gastrooesophageal reflux disease:0.08), (RANIMUSTINE:Gastrooesophageal reflux disease:0.26), (RASPBERRY KETONE:Gastrooesophageal reflux disease:0.33), (RED CLOVER:Gastrooesophageal reflux disease:0.86), (REISHI:Gastrooesophageal reflux disease:0.14), (RESERPINE:Gastrooesophageal reflux disease:0.01), (RHUBARB:Gastrooesophageal reflux disease:1.34), (RILMAZAFONE:Gastrooesophageal reflux disease:0.08), (RILPIVIRINE:Gastrooesophageal reflux disease:0.37), (RIPASUDIL HYDROCHLORIDE DIHYDRATE:Gastrooesophageal reflux disease:0.03), (ROPIVACAINE HYDROCHLORIDE:Gastrooesophageal reflux disease:0.38), (ROSIGLITAZONE:Gastrooesophageal reflux disease:0.1), (RUPATADINE FUMARATE:Gastrooesophageal reflux disease:1.03), (SAFFLOWER OIL:Gastrooesophageal reflux disease:0.08), (SAFINAMIDE MESYLATE:Gastrooesophageal reflux disease:0.07), (SEA WATER:Gastrooesophageal reflux disease:0.7), (SENNOSIDE A:Gastrooesophageal reflux disease:0.23), (SEROTONIN:Gastrooesophageal reflux disease:0.23), (SERTINDOLE:Gastrooesophageal reflux disease:0.5), (SHARK CARTILAGESHARK, UNSPECIFIED:Gastrooesophageal reflux disease:0.56), (SILTUXIMAB:Gastrooesophageal reflux disease:0.05), (SIVELESTAT SODIUM:Gastrooesophageal reflux disease:0.69), (SODIUM FLUORIDEVITAMINS:Gastrooesophageal reflux disease:0.67), (SODIUM GUALENATE MONOHYDRATE:Gastrooesophageal reflux disease:0.99), (SODIUM HYDROXIDE:Gastrooesophageal reflux disease:0.23), (SODIUM HYPOCHLORITE:Gastrooesophageal reflux disease:0.12), (SODIUM PHOSPHATE, DIBASIC:Gastrooesophageal reflux disease:0.71), (SODIUM PHOSPHATE, DIBASIC, ANHYDROUSSODIUM PHOSPHATE, DIBASIC, HEPTAHYDRATESODIUM PHOSPHATE, MONOBASIC, MONOHYDRATE:Gastrooesophageal reflux disease:1.35), (SOFALCONE:Gastrooesophageal reflux disease:0.55), (SOLCOSERYL:Gastrooesophageal reflux disease:0.04), (SOLIFENACINTAMSULOSIN:Gastrooesophageal reflux disease:0.05), (SOMATOSTATIN:Gastrooesophageal reflux disease:0.05), (SOMATREM:Gastrooesophageal reflux disease:0.44), (STREPTOMYCINSTREPTOMYCIN SULFATE:Gastrooesophageal reflux disease:0.48), (STRONTIUM RANELATE:Gastrooesophageal reflux disease:0.07), (SUCCINYLCHOLINE CHLORIDE:Gastrooesophageal reflux disease:0.47), (SUFENTANIL CITRATE:Gastrooesophageal reflux disease:1.56), (SULBUTIAMINE:Gastrooesophageal reflux disease:0.37), (SULFAMERAZINETRIMETHOPRIM:Gastrooesophageal reflux disease:0.48), (SULFAMETHAZINE:Gastrooesophageal reflux disease:0.18), (TABALUMAB:Gastrooesophageal reflux disease:0.66), (TAZAROTENE:Gastrooesophageal reflux disease:0.64), (TECELEUKIN:Gastrooesophageal reflux disease:0.06), (TEGAFURURACIL:Gastrooesophageal reflux disease:0.7), (TELOTRISTAT ETHYL:Gastrooesophageal reflux disease:2.04), (TEMOCAPRIL:Gastrooesophageal reflux disease:0.53), (TENECTEPLASE:Gastrooesophageal reflux disease:0.03), (TENOXICAM:Gastrooesophageal reflux disease:0.58), (TETANUS TOXOID, REDUCED DIPHTHERIA TOXOID AND ACELLULAR PERTUSSIS VACCINE ADSORBED, TDAP:Gastrooesophageal reflux disease:0.43), (TETRACAINE HYDROCHLORIDE:Gastrooesophageal reflux disease:0.63), (TETRAZEPAM:Gastrooesophageal reflux disease:0.74), (THIAMYLAL SODIUM:Gastrooesophageal reflux disease:0.09), (THIETHYLPERAZINE MALATE:Gastrooesophageal reflux disease:0.09), (THIOTHIXENE HYDROCHLORIDE:Gastrooesophageal reflux disease:0.47), (THYROGLOBULIN:Gastrooesophageal reflux disease:0.11), (TIAPRIDE:Gastrooesophageal reflux disease:0.27), (TIAPROFENIC ACID:Gastrooesophageal reflux disease:0.42), (TIOPRONIN:Gastrooesophageal reflux disease:0.8), (TIPEPIDINE HIBENZATE:Gastrooesophageal reflux disease:1.62), (TIROPRAMIDE:Gastrooesophageal reflux disease:0.33), (TOBRAMYCIN SULFATE:Gastrooesophageal reflux disease:0.28), (TOFOGLIFLOZIN:Gastrooesophageal reflux disease:0.19), (TOLPERISONE HYDROCHLORIDE:Gastrooesophageal reflux disease:0.01), (TRANILAST:Gastrooesophageal reflux disease:0.6), (TRANYLCYPROMINE:Gastrooesophageal reflux disease:0.09), (TRIMIPRAMINE MALEATE:Gastrooesophageal reflux disease:2.79), (TRIPTORELIN:Gastrooesophageal reflux disease:0.12), (TROGLITAZONE:Gastrooesophageal reflux disease:0.36), (TROLAMINE:Gastrooesophageal reflux disease:0.11), (TROXERUTIN:Gastrooesophageal reflux disease:0.26), (TULOBUTEROL HYDROCHLORIDE:Gastrooesophageal reflux disease:0.31), (UNDECYLENIC ACID:Gastrooesophageal reflux disease:0.35), (URACIL:Gastrooesophageal reflux disease:0.37), (VINBLASTINE SULFATE:Gastrooesophageal reflux disease:0.35), (VINORELBINEVINORELBINE TARTRATE:Gastrooesophageal reflux disease:0.64), (VITAMIN A PALMITATE:Gastrooesophageal reflux disease:0.83), (ZANAMIVIR:Gastrooesophageal reflux disease:0.77), (ZINC ACETATE:Gastrooesophageal reflux disease:0.87), (ZIV-AFLIBERCEPT:Gastrooesophageal reflux disease:0.7), (ADO-TRASTUZUMAB EMTANSINE:Gastrooesophageal reflux prophylaxis:0.28), (ALBUTEROL SULFATEIPRATROPIUM BROMIDE:Gastrooesophageal reflux prophylaxis:0.61), (ALCOHOL:Gastrooesophageal reflux prophylaxis:0.12), (AMISULPRIDE:Gastrooesophageal reflux prophylaxis:0.25), (ANASTROZOLE:Gastrooesophageal reflux prophylaxis:1.32), (ATOVAQUONE:Gastrooesophageal reflux prophylaxis:0.21), (BISACODYL OR DOCUSATE SODIUM:Gastrooesophageal reflux prophylaxis:0.49), (BLINATUMOMAB:Gastrooesophageal reflux prophylaxis:0.04), (BUDESONIDEFORMOTEROL FUMARATE:Gastrooesophageal reflux prophylaxis:0.11), (CALCITRIOL:Gastrooesophageal reflux prophylaxis:0.79), (CALCIUM ACETATE:Gastrooesophageal reflux prophylaxis:0.02), (CIMETIDINE:Gastrooesophageal reflux prophylaxis:0.06), (CLINDAMYCIN:Gastrooesophageal reflux prophylaxis:0.18), (CYANOCOBALAMINDEXPANTHENOLNIACINAMIDEPYRIDOXIN E HYDROCHLORIDERIBOFLAVIN 5′-PHOSPHATE SODIUMTHIAMINE HYDROCHLORIDEVITAMIN B COMPLEX:Gastrooesophageal reflux prophylaxis:0.04), (DABIGATRAN:Gastrooesophageal reflux prophylaxis:0.03), (DALTEPARIN:Gastrooesophageal reflux prophylaxis:0.33), (DASATINIB:Gastrooesophageal reflux prophylaxis:1.73), (DEXAMETHASONE ACETATE:Gastrooesophageal reflux prophylaxis:0.09), (DIGITOXIN:Gastrooesophageal reflux prophylaxis:0.22), (DIPYRONE:Gastrooesophageal reflux prophylaxis:0.03), (DOFETILIDE:Gastrooesophageal reflux prophylaxis:0.09), (ECONAZOLE:Gastrooesophageal reflux prophylaxis:0.06), (ELOTUZUMAB:Gastrooesophageal reflux prophylaxis:0.18), (EPIRUBICIN:Gastrooesophageal reflux prophylaxis:0.72), (EVOLOCUMAB:Gastrooesophageal reflux prophylaxis:0.31), (EZETIMIBESIMVASTATIN:Gastrooesophageal reflux prophylaxis:0.1), (FENOFIBRATE:Gastrooesophageal reflux prophylaxis:0.86), (FERROUS SULFATEFERROUS SULFATE, DRIED:Gastrooesophageal reflux prophylaxis:1.65), (FORMOTEROL FUMARATE:Gastrooesophageal reflux prophylaxis:0.22), (FUSIDIC ACID:Gastrooesophageal reflux prophylaxis:0), (GLIMEPIRIDE:Gastrooesophageal reflux prophylaxis:1.26), (GLYCOPYRRONIUMINDACATEROL:Gastrooesophageal reflux prophylaxis:0.45), (ILOPROST:Gastrooesophageal reflux prophylaxis:0.21), (IPRATROPIUM:Gastrooesophageal reflux prophylaxis:0.55), (LEDIPASVIRSOFOSBUVIR:Gastrooesophageal reflux prophylaxis:1.23), (LERCANIDIPINE HYDROCHLORIDE:Gastrooesophageal reflux prophylaxis:0.86), (LEVALBUTEROL HYDROCHLORIDE:Gastrooesophageal reflux prophylaxis:0.23), (LEVOLEUCOVORIN CALCIUM:Gastrooesophageal reflux prophylaxis:0.29), (METHADONE HYDROCHLORIDE:Gastrooesophageal reflux prophylaxis:0.28), (METHYLPREDNISOLONE ACETATE:Gastrooesophageal reflux prophylaxis:0.17), (NEBIVOLOL HYDROCHLORIDE:Gastrooesophageal reflux prophylaxis:0.24), (OCTREOTIDE ACETATE:Gastrooesophageal reflux prophylaxis:0.07), (OMEGA-3-ACID ETHYL ESTERS:Gastrooesophageal reflux prophylaxis:0.18), (PANTOPRAZOLE MAGNESIUM:Gastrooesophageal reflux prophylaxis:0.69), (PAZOPANIB HYDROCHLORIDE:Gastrooesophageal reflux prophylaxis:0.87), (PITAVASTATIN CALCIUM:Gastrooesophageal reflux prophylaxis:0.39), (PRAMIPEXOLE DIHYDROCHLORIDE:Gastrooesophageal reflux prophylaxis:0.05), (PRIMIDONE:Gastrooesophageal reflux prophylaxis:0.41), (PROBIOTICS NOS:Gastrooesophageal reflux prophylaxis:0.05), (PROMETHAZINE HYDROCHLORIDE:Gastrooesophageal reflux prophylaxis:0.14), (RISEDRONATE SODIUM:Gastrooesophageal reflux prophylaxis:0.34), (ROFLUMILAST:Gastrooesophageal reflux prophylaxis:0.22), (SACUBITRILVALSARTAN:Gastrooesophageal reflux prophylaxis:0.85), (TIOTROPIUM:Gastrooesophageal reflux prophylaxis:0.37), (TOCILIZUMAB:Gastrooesophageal reflux prophylaxis:0.41), (TOPIRAMATE:Gastrooesophageal reflux prophylaxis:0.67), (VITAMIN B:Gastrooesophageal reflux prophylaxis:0.15), (AMITRIPTYLINE:Gaucher's disease:0.08), (CELECOXIB:Gaucher's disease:0.39), (CIPROFLOXACIN HYDROCHLORIDE:Gaucher's disease:0.11), (CLINDAMYCINCLINDAMYCIN PHOSPHATE:Gaucher's disease:0.01), (DEVICE:Gaucher's disease:0.02), (DICLOFENAC SODIUM:Gaucher's disease:0.2), (DILTIAZEM:Gaucher's disease:0.12), (DIVALPROEX SODIUM:Gaucher's disease:0.58), (ENOXAPARIN:Gaucher's disease:0.33), (EVOLOCUMAB:Gaucher's disease:0.2), (HYDROCODONE:Gaucher's disease:0.09), (IBRUTINIB:Gaucher's disease:0.54), (INDOMETHACIN:Gaucher's disease:0.06), (PRAVASTATIN:Gaucher's disease:0.03), (PRIMIDONE:Gaucher's disease:0.07), (PROMETHAZINEPROMETHAZINE HYDROCHLORIDE:Gaucher's disease:0.03), (RAMIPRIL:Gaucher's disease:0.17), (RANOLAZINE:Gaucher's disease:0.01), (SODIUM OXYBATE:Gaucher's disease:0.36), (TOCILIZUMAB:Gaucher's disease:0.12), (VERAPAMIL HYDROCHLORIDE:Gaucher's disease:0.32), (LACOSAMIDE:Gene mutation:0.03), (ALCOHOL:General anaesthesia:0.26), (BUMETANIDE:General anaesthesia:0.22), (CALCIUM CARBONATECHOLECALCIFEROL:General anaesthesia:0.28), (CARBON DIOXIDE:General anaesthesia:0.19), (CERTOLIZUMAB PEGOL:General anaesthesia:0.12), (CORTISONEHYDROCORTISONE:General anaesthesia:0.02), (CYCLOBENZAPRINE HYDROCHLORIDE:General anaesthesia:0.01), (DILTIAZEM:General anaesthesia:0.43), (EVEROLIMUS:General anaesthesia:0.09), (FLECAINIDE:General anaesthesia:0.13), (FLUTICASONE PROPIONATE:General anaesthesia:0.54), (HYDROXYZINE PAMOATE:General anaesthesia:0.06), (IBRUTINIB:General anaesthesia:0.2), (METARAMINOL:General anaesthesia:0.27), (METHADONE HYDROCHLORIDE:General anaesthesia:0.18), (MOXIFLOXACIN HYDROCHLORIDE:General anaesthesia:0.12), (NAPROXEN:General anaesthesia:0.49), (NEBIVOLOL:General anaesthesia:0.49), (OXCARBAZEPINE:General anaesthesia:1.38), (PENTOBARBITAL:General anaesthesia:0.54), (PRAMIPEXOLE:General anaesthesia:0.22), (PRAMIPEXOLE DIHYDROCHLORIDE:General anaesthesia:0.07), (PRAVASTATIN SODIUM:General anaesthesia:0.24), (PROMETHAZINE HYDROCHLORIDE:General anaesthesia:0.09), (SOTALOL:General anaesthesia:0.07), (TERIFLUNOMIDE:General anaesthesia:0.04), (TIZANIDINE HYDROCHLORIDE:General anaesthesia:0.15), (TORSEMIDE:General anaesthesia:0.54), (VALGANCICLOVIR HYDROCHLORIDE:General anaesthesia:0.12), (VARENICLINE TARTRATE:General anaesthesia:0.12), (VERAPAMIL HYDROCHLORIDE:General anaesthesia:0.26), (AMPHETAMINE ASPARTATEAMPHETAMINE SULFATEDEXTROAMPHETAMINE SACCHARATEDEXTROAMPHETAMINE SULFATE:General physical condition:0.07),(ARMODAFINIL:General physical condition:0.01), (BUSPIRONE HYDROCHLORIDE:General physical condition:0.24), (CANDESARTAN CILEXETIL:General physical condition:0.06), (CERTOLIZUMAB PEGOL:General physical condition:0.43), (CLONIDINE:General physical condition:0.1), (DEVICE:General physical condition:0.31), (EVOLOCUMAB:General physical condition:0.7), (ILOPROST:General physical condition:0.1), (INDOMETHACIN:General physical condition:0.08), (LOVASTATIN:General physical condition:0.37), (METAXALONE:General physical condition:0.11), (METHYLPREDNISOLONE SODIUM SUCCINATE:General physical condition:0.02), (METRONIDAZOLE:General physical condition:0.21), (PRAVASTATIN SODIUM:General physical condition:0.21), (RALOXIFENE HYDROCHLORIDE:General physical condition:0.1), (RANOLAZINE:General physical condition:0.23), (SACUBITRILVALSARTAN:General physical condition:0.81), (SODIUM OXYBATE:General physical condition:0.05), (TRIAMCINOLONE ACETONIDE:General physical condition:0.02), (VARENICLINE TARTRATE:General physical condition:0.13), (ACETAMINOPHENCODEINE PHOSPHATE:General physical health deterioration:0.19), (ANASTROZOLE:General physical health deterioration:0.48), (BUMETANIDE:General physical health deterioration:0.39), (CANDESARTAN:General physical health deterioration:0.03), (CANDESARTAN CILEXETIL:General physical health deterioration:0.1), (CLINDAMYCINCLINDAMYCIN PHOSPHATE:General physical health deterioration:0.16), (DALFAMPRIDINE:General physical health deterioration:0.07), (DEXLANSOPRAZOLE:General physical health deterioration:0.2), (DILTIAZEM:General physical health deterioration:0.25), (DIVALPROEX SODIUM:General physical health deterioration:0.18), (EVOLOCUMAB:General physical health deterioration:0.11), (IBRUTINIB:General physical health deterioration:0.64), (INSULIN ASPART:General physical health deterioration:0.32), (ISOSORBIDE MONONITRATE:General physical health deterioration:0.26), (NAPROXEN:General physical health deterioration:0.69), (NEBIVOLOL:General physical health deterioration:0.18), (NILOTINIB:General physical health deterioration:0.21), (PAROXETINE:General physical health deterioration:0.19), (RISEDRONATE SODIUM:General physical health deterioration:0.01), (TERIFLUNOMIDE:General physical health deterioration:0.32), (TOPIRAMATE:General physical health deterioration:0.51), (TRIAMCINOLONE ACETONIDE:General physical health deterioration:0.21), (VARENICLINE TARTRATE:General physical health deterioration:0.1), (AMIODARONE:Generalised anxiety disorder:0.27), (AMISULPRIDE:Generalised anxiety disorder:0.23), (CALCIUM CARBONATECHOLECALCIFEROL:Generalised anxiety disorder:0.86), (CALCIUM CITRATE:Generalised anxiety disorder:0.09), (CANDESARTAN CILEXETIL:Generalised anxiety disorder:0.11), (CERTOLIZUMAB PEGOL:Generalised anxiety disorder:0.77), (CHLORPROMAZINE:Generalised anxiety disorder:0.09), (CHOLESTYRAMINE:Generalised anxiety disorder:0.21), (CLINDAMYCINCLINDAMYCIN PHOSPHATE:Generalised anxiety disorder:0.04), (DALFAMPRIDINE:Generalised anxiety disorder:0.11), (DIMENHYDRINATE:Generalised anxiety disorder:0.09), (ENOXAPARIN:Generalised anxiety disorder:0.21), (ESTROGENS, CONJUGATEDMEDROXYPROGESTERONE ACETATE:Generalised anxiety disorder:0.1), (EVOLOCUMAB:Generalised anxiety disorder:0.82), (GUANFACINE:Generalised anxiety disorder:0.12), (IBRUTINIB:Generalised anxiety disorder:0.75), (IMIPRAMINE HYDROCHLORIDE:Generalised anxiety disorder:0.14), (INSULIN GLARGINE:Generalised anxiety disorder:0.41), (INSULIN LISPRO:Generalised anxiety disorder:0.26), (LABETALOLLABETALOL HYDROCHLORIDE:Generalised anxiety disorder:0.01), (LEDIPASVIRSOFOSBUVIR:Generalised anxiety disorder:0.25), (LIDOCAINE:Generalised anxiety disorder:0.28), (METRONIDAZOLE:Generalised anxiety disorder:0.79), (NITROFURANTOIN:Generalised anxiety disorder:0.2), (OSPEMIFENE:Generalised anxiety disorder:0.12), (PENICILLIN:Generalised anxiety disorder:0.19), (PRIMIDONE:Generalised anxiety disorder:0.33), (RABEPRAZOLE SODIUM:Generalised anxiety disorder:0.04), (RANOLAZINE:Generalised anxiety disorder:0.18), (RIZATRIPTAN BENZOATE:Generalised anxiety disorder:0.62), (ROPINIROLE HYDROCHLORIDE:Generalised anxiety disorder:0.22), (SACUBITRILVALSARTAN:Generalised anxiety disorder:0.06), (SECUKINUMAB:Generalised anxiety disorder:0.22), (SUMATRIPTANSUMATRIPTAN SUCCINATE:Generalised anxiety disorder:1.1), (TELMISARTAN:Generalised anxiety disorder:0.05), (TERIFLUNOMIDE:Generalised anxiety disorder:0.4), (UMECLIDINIUM BROMIDEVILANTEROL TRIFENATATE:Generalised anxiety disorder:0.05), (VITAMIN B:Generalised anxiety disorder:0.22), (ALCOHOL:Generalised tonic-clonic seizure:0.32), (ATRACURIUM:Generalised tonic-clonic seizure:0.06), (BUMETANIDE:Generalised tonic-clonic seizure:0.41), (BUPRENORPHINE HYDROCHLORIDE:Generalised tonic-clonic seizure:0.33), (BUPROPION:Generalised tonic-clonic seizure:0.49), (BUPROPION HYDROCHLORIDE:Generalised tonic-clonic seizure:1.55), (BUSPIRONE HYDROCHLORIDE:Generalised tonic-clonic seizure:0.57), (CEPHALEXIN:Generalised tonic-clonic seizure:0.33), (CERTOLIZUMAB PEGOL:Generalised tonic-clonic seizure:0.5), (CHOLESTYRAMINE:Generalised tonic-clonic seizure:0.22), (DESFLURANE:Generalised tonic-clonic seizure:0.79), (DEXMEDETOMIDINE HYDROCHLORIDE:Generalised tonic-clonic seizure:0.19), (DICLOFENAC SODIUM:Generalised tonic-clonic seizure:0.61), (DILTIAZEM:Generalised tonic-clonic seizure:0.26), (ESZOPICLONE:Generalised tonic-clonic seizure:0.63), (EVOLOCUMAB:Generalised tonic-clonic seizure:0.45), (FERROUS SULFATE:Generalised tonic-clonic seizure:0.74), (HYDROMORPHONE HYDROCHLORIDE:Generalised tonic-clonic seizure:0.66), (HYDROXYCHLOROQUINE SULFATE:Generalised tonic-clonic seizure:0.05), (IBRUTINIB:Generalised tonic-clonic seizure:1.23), (ILOPROST:Generalised tonic-clonic seizure:0.06), (IPRATROPIUM:Generalised tonic-clonic seizure:0.06), (METHYLPREDNISOLONE ACETATE:Generalised tonic-clonic seizure:0.54), (METRONIDAZOLEMETRONIDAZOLE HYDROCHLORIDE:Generalised tonic-clonic seizure:0.11), (MOXIFLOXACIN HYDROCHLORIDE:Generalised tonic-clonic seizure:0.1), (NALOXONE HYDROCHLORIDEOXYCODONE HYDROCHLORIDE:Generalised tonic-clonic seizure:0), (NORTRIPTYLINE:Generalised tonic-clonic seizure:0.14), (OXYCODONE HYDROCHLORIDE:Generalised tonic-clonic seizure:0.05), (PANTOPRAZOLE:Generalised tonic-clonic seizure:0.99), (PAROXETINE:Generalised tonic-clonic seizure:0.54), (PAROXETINEPAROXETINE HYDROCHLORIDE:Generalised tonic-clonic seizure:0.55), (PEGINTERFERON BETA-1A:Generalised tonic-clonic seizure:0), (PRAMIPEXOLE DIHYDROCHLORIDE:Generalised tonic-clonic seizure:0.27), (RANOLAZINE:Generalised tonic-clonic seizure:0.25), (RIZATRIPTAN BENZOATE:Generalised tonic-clonic seizure:0.17), (SUMATRIPTANSUMATRIPTAN SUCCINATE:Generalised tonic-clonic seizure:0.07), (TERIFLUNOMIDE:Generalised tonic-clonic seizure:0.54), (TETRACYCLINE:Generalised tonic-clonic seizure:0.14), (TOCILIZUMAB:Generalised tonic-clonic seizure:0.12), (TOFACITINIB CITRATE:Generalised tonic-clonic seizure:0.05), (VALGANCICLOVIR HYDROCHLORIDE:Generalised tonic-clonic seizure:0.04), (VARENICLINE TARTRATE:Generalised tonic-clonic seizure:1.06), (VECURONIUM BROMIDE:Generalised tonic-clonic seizure:0.52), (VITAMIN B:Generalised tonic-clonic seizure:0.09), (CALCIUM CARBONATECHOLECALCIFEROL:Genital herpes:0.13), (DALFAMPRIDINE:Genital herpes:0.19), (DIPHENHYDRAMINE:Genital herpes:0.28), (DULOXETINE:Genital herpes:0.06), (EVOLOCUMAB:Genital herpes:0.02), (FERROUS SULFATE:Genital herpes:0.15), (FERROUS SULFATEFERROUS SULFATE, DRIED:Genital herpes:0.54), (INSULIN NOS:Genital herpes:0.24), (METAXALONE:Genital herpes:0.09), (METRONIDAZOLE:Genital herpes:0.84), (PRAVASTATIN SODIUM:Genital herpes:0.09), (PROGESTERONE:Genital herpes:0.07), (SACUBITRILVALSARTAN:Genital herpes:0.04), (TAMSULOSIN:Genital herpes:0.06), (TERIFLUNOMIDE:Genital herpes:0.6), (VERAPAMIL HYDROCHLORIDE:Genital herpes:0.21), (.ALPHA.1-PROTEINASE INHIBITOR HUMAN:Glaucoma:0.37), (ACETAMINOPHENCAFFEINE:Glaucoma:0.62), (ACETAMINOP HENCHLORPHENIRAMINE MALEATEPSEUDOEPHEDRINE HYDROCHLORIDE:Glaucoma:0.22), (ACETAMINOPHENDIPHENHYDRAMINE HYDROCHLORIDE:Glaucoma:0.27), (ACETAMINOPHENMETHOCARBAMOL:Glaucoma: 0.16), (ADO-TRASTUZUMAB EMTANSINE:Glaucoma:1.51), (AGALSIDASE BETA:Glaucoma:1.47), (ALBIGLUTIDE:Glaucoma:0.34), (AMBROXOL:Glaucoma:0.27), (AMI NO ACIDSDEXTROSEELECTROLYTES NOS:Glaucoma:0.14), (AMISULPRIDE:Glaucoma:1.22), (AMLODIPINE BESYLATETELMISARTAN:Glaucoma:0.87), (AMMONIUM LACTATE:Glaucoma:1.17), (ASCORBIC ACIDBIOTINCALCIUM PANTOTHENATECOBALAMINFOLIC ACIDNIACINAMIDEPYRIDOXINE HYDROCHLORIDERIBOFLAVINTHIAMINE MONONITRATE:Glaucoma:0.35), (ASCORBIC ACIDBIOTINCYANOCOBALAMINFOLIC ACIDNIACINPANTOTHENIC ACIDPYRIDOXINERIBOFLAVINTHIAMINE:Glaucoma:0.34), (ASPIRINBUTALBITAL CAFFEINE:Glaucoma:0.03), (ASPIRINCLOPIDOGREL:Glaucoma:0.27), (ASUNAPREVIR: Glaucoma:0.62), (ATORVASTATIN CALCIUMEZETIMIBE:Glaucoma:0.01), (ATOVAQUONE:Glaucoma:0.72), (AVANAFIL:Glaucoma: 0.04), (AZELNIDIPINE:Glaucoma:0.53), (BECLOMETHASONE:Glaucoma:0.36), (BEC LOMETHASONE DIPROPIONATEFORMOTEROL:Glaucoma:0.07), (BELATACEPT:Glaucoma:0.18), (BEPOT ASTINE BESILATE:Glaucoma:0.42), (BEPRIDIL:Glaucoma:0.64), (BETAMETHASONE DIPROPIONATECLOTRIMAZOLE:Glaucoma:0.84), (BETAMETHASONECALCIPOTRIEN E:Glaucoma:0.77), (BETHANECHOL CHLORIDE:Glaucoma:0.53), (BILASTINE:Glaucoma:0.64), (BIPERIDEN HYDROCHLORIDE:Glaucoma:0.27), (BLINATUMOMAB:Glaucoma:0.26), (BOCEPREVIR: Glaucoma:0.1), (BOTULINUM TOXIN TYPE A:Glaucoma:0.44), (BROMFENACBROMFENAC SODIUM:Glaucoma:0.16), (BUCILLAMINE:Glaucoma:0.01), (BUPIVACAINE HYDROCHLORIDE:Glaucoma:0.13), (BUPRENORPHINE HYDROCHLORIDE:Glaucoma:1.26), (CABAZITAXEL:Glaucoma:0.12), (CALCIUM LACTATE:Glaucoma:0.2), (CALCIUM PANTOTHENATECYANOCOBALAMINNIACINAMIDEPYRIDOXINE HYDROCHLORIDERIBOFLAVINTHIAMINE MONONITRATE:Glaucoma:0.3), (CALCIUMMAGNESIUMZINC:Glaucoma:1.38), (CANAG LIFLOZINMETFORMIN HYDROCHLORIDE:Glaucoma:1.66), (CASPOFUNGIN:Glaucoma:0.1), (CEFOTAXIME SODIUM:Glaucoma:0.84), (CERIVASTATIN SODIUM:Glaucoma:0.26), (CETUXIMAB:Glaucoma:1.05), (CHLORDIAZEPOXIDE:Glaucoma: 0.24), (CHOLESTEROL:Glaucoma:0.15), (CIMETIDINE:Glaucoma:2.56), (CLEMASTINE FUMARATE:Glaucoma:0.04), (CLINDAMYCIN HYDROCHLORIDE:Glaucoma:0.51), (COBICISTATELVITEGRAVIREMTRICITABINET ENOFOVIR ALAFENAMIDE FUMARATE:Glaucoma:0.04), (CODEINE PHOSPHATEGUAIFENESIN:Glaucoma:0.41), (CONVALLARIA MAJALISCRATAEGUS LAEVIGATA LEAFPROXYPHYLLINE:Glaucoma:0.09), (CYPROHEPTADINE HYDROCHLORIDE:Glaucoma:0.06), (DACLIZUMAB:Glaucoma:1.42), (DANTROLENE SODIUM:Glaucoma:0.2), (DEGARELIX ACETATE:Glaucoma:0.05), (DESMOPRESSIN:Glaucoma:0.2), (DEXAMETHASONENEOM YCINPOLYMYXIN B SULFATE:Glaucoma:0.6), (DEXTRAN 70\GLYCERINHYPROMELLOSES:Glaucoma:0.19), (DEXTROAMPHETAMINE:Glaucoma: 0.07), (DEXTROAMPHETAMINE SULFATE:Glaucoma:0.99), (DICLOFENAC SODIUMMISOPROSTOL:Glaucoma:0.49), (DOLASETRON MESYLATE:Glaucoma:0.63), (ECONAZOLE:Glaucoma:1.46), (ELTROMBOPAG OLAMINE:Glaucoma:0.07), (EPERISONE HYDROCHLORIDE:Glaucoma:0.14), (ERIBULIN:Glaucoma:0.32), (ESTRIOL:Glaucoma:0.76), (ESTROGENS:Glaucoma:0.7), (ETHACRYNIC ACID:Glaucoma:0.33), (ETHINYL ESTRADIOLNORGESTREL:Glaucoma:0.12), (ETRETINATE:Glaucoma:1.31), (FERROUS GLYCINE SULFATE:Glaucoma:0.24), (FERROUS SULFATEFOLIC ACID:Glaucoma:0.29), (FERROUS SULFATEGLYCINE:Glaucoma:0.58), (FLUNARIZINE HYDROCHLORIDE:Glaucoma:0.25), (FLUTICASONE PROPIONATEFORMOTEROL FUMARATE:Glaucoma:0.03), (FONDAPARINUX:Glaucoma:0.41), (FUSIDIC ACID:Glaucoma:1.71), (GADOPENTETATE DIMEGLUMINE:Glaucoma:0.4), (GEFITINIB:Glaucoma:0.36), (GENTAMICIN SULFATE:Glaucoma:0.2), (GIMERACILOTERACILTEGAFUR:Glaucoma:1.18), (GINGER: Glaucoma:0.18), (GLIMEPIRIDEROSIGLITAZONE MALEATE:Glaucoma:0.24), (GLYCOPYRROLATEINDACATEROL MALEATE:Glaucoma:0.71), (GUANFACINE HYDROCHLORIDE:Glaucoma:0.42), (IDARUCIZUMAB:Glaucoma:0.02), (ILOPROST:Glauc oma:1.3), (IMIDAPRIL:Glaucoma:0.12), (IMIGLUCERASE:Glaucoma:0.39), (IMIQUIMOD:Gla ucoma:0.25), (INDACATEROL:Glaucoma:1.52), (INFLIXIMAB-DYYB:Glaucoma:0.74), (INSULIN ASPARTINSULIN DEGLUDEC:Glaucoma:0.11), (INTERFERON:Glaucoma:0.98), (INTERFERON ALFA-2A:Glaucoma:1.34), (INTERFERON ALFA-2B:Glaucoma:0.86), (IRINOTECAN:Glaucoma:0.6), (IRINOTECAN HYDROCHLORIDE:Glaucoma:1.33), (IRONPOLYSACCHARIDES:Glaucoma:0.23), (IVABR ADINE HYDROCHLORIDE:Glaucoma:0.34), (LECITHIN:Glaucoma:0.12), (LENVATINIB:Glaucoma: 0.58), (LEVOMEFOLATE CALCIUM:Glaucoma:0.37), (LINSIDOMINE:Glaucoma:0.13), (LIOTHYRONINE SODIUM:Glaucoma:1.36), (LOMITAPIDE MESYLATE:Glaucoma:1), (LUTETIUM LU-177:Glaucoma:0.39), (MAGNESIUM ASPARTATE HYDROCHLORIDE:Glaucoma:0.23), (MELPERONE:Glaucoma:0.42), (MEPERIDINE:Glauco ma:0.97), (MEPOLIZUMAB:Glaucoma:0.82), (MESNA:Glaucoma:0.57), (METFORMIN HYDROCHLORIDEROSIGLITAZONE MALEATE:Glaucoma:0.48), (METHENAMINE MANDELATE:Glaucoma:0.04), (METHENOLONE ENANTHATE:Glaucoma:0.43), (METHYLPREDNISOLONE ACETATE:Glaucoma:2.33), (METRELEPTIN:Glaucoma:0.06), (MICONAZOLE NITRATE:Glaucoma:0.44), (MIDODRINE HYDROCHLORIDE:Glaucoma:0.76), (MOEXIPRIL HYDROCHLORIDE:Glaucoma:0.91), (NALTREXONE:Glaucoma:0.8), (NAPHAZOLINE HYDROCHLORIDEPHENIRAMINE MALEATE:Glaucoma:0.31), (NATAMYCIN:Glaucoma:0.06), (NISOLDIPINE:Glaucoma:0.19), (NOREPINEPHRINE:Glaucoma:0.01), (NORETHINDRONE:Glaucoma:0.12), (OCTREOTIDE: Glaucoma:1.47), (OPIPRAMOL:Glaucoma:0.28), (OSELTAMIVIR:Glaucoma:0.88), (PANTOPR AZOLE MAGNESIUM:Glaucoma:2.63), (PATIROMER:Glaucoma:0.82), (PEGINTERFERON BETA-1A:Glaucoma:1.22), (PEMETREXED:Glaucoma:1.13), (PENICILLIN G:Glaucoma:0.54), (PENTAMIDINE ISETHIONATE:Glaucoma:0.06), (PENTOSTATIN:Glaucoma:0.22), (PERTUZUMAB:Glaucom a:0.56), (POLYETHYLENE GLYCOL 3350\POTASSIUM CHLORIDESODIUM BICARBONATESODIUM CHLORIDESODIUM SULFATE ANHYDROUS:Glaucoma:0.51), (POLYVINYL ALCOHOL:Glaucoma:0.16), (PRANLUKAST:Glaucoma:0.41), (PRAZOSIN:Glaucoma:1.11), (P REDNISOLONE SODIUM PHOSPHATE:Glaucoma:0.12), (PREDNISOLONE SODIUM SUCCINATE:Glaucoma:0), (PRISTINAMYCIN:Glaucoma:0.62), (PROTRIPTYLINE HYDROCHLORIDE:Glaucoma:0.59), (PYRAZINAMIDE:Glaucoma:0.78), (QUINACRINE:Gla ucoma:0.28), (RAMUCIRUMAB:Glaucoma:0.64), (ROFECOXIB:Glaucoma:0), (RUCAPARIB CAMSYLATE:Glaucoma:0.03), (SELEGILINE:Glaucoma:0.52), (SELEXIPAG:Glaucoma:0.82), (SENNA LEAF:Glaucoma:0.2), (SIPULEUCEL-T:Glaucoma:0.2), (SODIUM PHOSPHATE, DIBASICSODIUM PHOSPHATE, MONOBASIC:Glaucoma:0.58), (SOFOSBUVIRVELPATASVIR:Glaucoma:0.29), (STARCH, WHEAT:Glaucoma:0.48), (SUPLATAST TOSILATE:Glaucoma:0.13), (TELOTRISTAT ETHYL:Glaucoma:0.34), (TEMSIROLIMUS:Glaucoma:0.14), (TEPRENONE:Glaucoma:0.78), (TESTOSTERONE UNDECANOATE:Glaucoma:1.35), (TETRACAINE HYDROCHLORIDE:Glaucoma:0.71), (TETRACAINETETRACAINE HYDROCHLORIDE:Glaucoma:0.71), (THEOPHYLLINE:Glaucoma:0.06), (THIOCTIC ACID:Glaucoma:0.64), (TICLOPIDINE:Glaucoma:1.03), (TIPIRACIL HYDROCHLORIDETRIFLURIDINE:Glaucoma:0.57), (TRIPTORELIN PAMOATE:Glaucoma:0.76), (VALDECOXIB:Glaucoma:0.34), (VINORELBINE TARTRATE:Glaucoma:0.8), (VINORELBINEVINORELBINE TARTRATE:Glaucoma:0.05), (WARFARIN POTASSIUM:Glaucoma:0.79), (ZILEUTON:Glaucoma:0.47), (ZIV-AFLIBERCEPT:Glaucoma:0.06), (ACETAMINOPHENCODEINE PHOSPHATE:Glioblastoma:0.51), (ALBUTEROL SULFATEIPRATROPIUM BROMIDE:Glioblastoma:0.01), (AMIODARONE:Glioblastoma:0.53), (AMOXICILLIN:Gliobla stoma:0.56), (ANASTROZOLE:Glioblastoma:0.74), (BUMETANIDE:Glioblastoma:0.33), (CAL CITRIOL:Glioblastoma:0.61), (CANDESARTAN CILEXETILHYDROCHLOROTHIAZIDE:Glioblastoma:0.24), (CERTOLIZUMAB PEGOL:Glioblastoma:0.35), (CETUXIMAB:Glioblastoma:0.21), (CLOZAPINE:Glioblastoma:0.21), (CYCLOBENZAPRINE:Glioblastoma:0.09), (DEXLANSOPRAZOLE:Glioblastoma:0.76), (DICLOFENAC SODIUM:Glioblastoma:0.5), (DICYCLOMINE HYDROCHLORIDE:Glioblastoma:0), (DILTIAZEM:Glioblastoma:0.21), (DOXAZOSINDOX AZOSIN MESYLATE:Glioblastoma:0.04), (EPINEPHRINE:Glioblastoma:0.4), (ERYTHROPOIETIN:Gli oblastoma:0.24), (EVOLOCUMAB:Glioblastoma:0.04), (FERROUS SULFATE:Glioblastoma:0.12), (FERROUS SULFATEFERROUS SULFATE, DRIED:Glioblastoma:0.93), (GADOVERSETAMIDE:Glioblastoma:0.37), (IBRUTINIB:Glioblas toma:0.85), (LENALIDOMIDE:Glioblastoma:0.46), (LEVOLEUCOVORIN CALCIUM:Glioblastoma:0.31), (LINAGLIPTIN:Glioblastoma:0.32), (MELOXICAM:Glioblasto ma:0.32), (METRONIDAZOLE:Glioblastoma:0.36), (OXYGEN:Glioblastoma:0.01), (PRAMIPE XOLE:Glioblastoma:0.12), (PRAVASTATIN:Glioblastoma:0.56), (RANOLAZINE:Glioblastoma:0.05), (SACUBITRILVALSARTAN:Glioblastoma:0.12), (SOTALOL:Glioblastoma:0.29), (TIM OLOL:Glioblastoma:0.42), (TIZANIDINE HYDROCHLORIDE:Glioblastoma:0.19), (ANASTROZOLE:Glioblastoma multiforme:0.18), (BUPROPION HYDROCHLORIDE:Glioblastoma multiforme:0.08), (CALCIUM CARBONATECHOLECALCIFEROL:Glioblastoma multiforme:0.17), (IBRUTINIB:Glioblastoma multiforme:0.7), (IMATINIB MESYLATE:Glioblastoma multiforme:0.07), (LOVASTATIN:Glioblastoma multiforme:0.15), (MAGNESIUM:Glioblastoma multiforme:0.08), (NAPROXEN:Glioblastoma multiforme:0.28), (NILOTINIB:Glioblastoma multiforme:0.06), (POTASSIUM:Glioblastoma multiforme:0.2), (PRAVASTATIN:Glioblastoma multiforme:0.02), (PRIMIDONE:Glioblastoma multiforme:0.08), (TAMSULOSIN:Glioblastoma multiforme:0.11), (TORSEMIDE:Glioblastoma multiforme:0.26), (TRIAMCINOLONE ACETONIDE:Glioblastoma multiforme:0.19), (VERAPAMIL HYDROCHLORIDE:Glioblastoma multiforme:0.26), (LAMOTRIGINE:Glioma:0.59), (ACETAMINOPHENBUTALBITALCAFF EINE:Glucose tolerance impaired:0.8), (ACETAMINOPHENOXYCODONEOXYCODONE HYDROCHLORIDE:Glucose tolerance impaired:0.19), (ALCOHOL:Glucose tolerance impaired:0.06), (AMIODARONE:Glucose tolerance impaired:1.29), (ARIPIPRAZOLE LAUROXIL:Glucose tolerance impaired:0.1), (ATROPINE SULFATEDIPHENOXYLATE HYDROCHLORIDE:Glucose tolerance impaired:0.42), (AZITHROMYCIN ANHYDROUS:Glucose tolerance impaired:0.18), (BRINZOLAMIDE:Glucose tolerance impaired:0.48), (BUPRENORPHINE HYDROCHLORIDENALOXONE HYDROCHLORIDE:Glucose tolerance impaired:0.21), (CALCIUM ACETATE:Glucose tolerance impaired:0.8), (CARFILZOMIB:Glucose tolerance impaired:0.06), (CARVEDILOL PHOSPHATE:Glucose tolerance impaired:1.09), (CLINDAMYCIN:Glucose tolerance impaired:0.29), (CLINDAMYCINCLINDAMYCIN PHOSPHATE:Glucose tolerance impaired:0.42), (CODEINE:Glucose tolerance impaired:0.16), (DABIGATRAN:Glucose tolerance impaired:0.07), (DACLIZUMAB:Glucose tolerance impaired:0.79), (DASATINIB:Glucose tolerance impaired:0.95), (DEFLAZACORT:Glucose tolerance impaired:0.02), (DESLORATADINE:Glucose tolerance impaired:0.52), (DEVICE:Glucose tolerance impaired:1.42), (DIMENHYDRINATE:Glucose tolerance impaired:0.1), (DUTASTERIDE:Glucose tolerance impaired:1.06), (ELETRIPTAN HYDROBROMIDE:Glucose tolerance impaired:0.82), (EMPAGLIFLOZINMETFORMIN HYDROCHLORIDE:Glucose tolerance impaired:0.23), (ENOXAPARIN:Glucose tolerance impaired:0.88), (ENOXAPARIN SODIUM:Glucose tolerance impaired:0.34), (EPINEPHRINE:Glucose tolerance impaired:0.6), (ESTROGENS, CONJUGATEDMEDROXYPROGESTERONE ACETATE:Glucose tolerance impaired:0.14), (FERROUS SULFATE:Glucose tolerance impaired:1.28), (FESOTERODINE FUMARATE:Glucose tolerance impaired:0.63), (FLUCONAZOLE:Glucose tolerance impaired:0.85), (FLUTICASONE:Glucose tolerance impaired:0.54), (FLUTICASONE FUROATE:Glucose tolerance impaired:0.11), (FULVESTRANT:Glucose tolerance impaired:0.44), (GLYCOPYRRONIUMINDACATEROL:Glucose tolerance impaired:0.07), (HYDROCHLOROTHIAZIDEIRBESARTAN:Glucose tolerance impaired:0.5), (HYDROMORPHONE:Glucose tolerance impaired:0.21), (HYDROMORPHONE HYDROCHLORIDE:Glucose tolerance impaired:0.61), (HYDROXYCHLOROQUINE DIPHOSPHATE:Glucose tolerance impaired:0.23), (HYOSCYAMINE:Glucose tolerance impaired:0.69), (ILOPROST:Glucose tolerance impaired:0), (IMATINIB MESYLATE:Glucose tolerance impaired:0.35), (INFLUENZA VIRUS VACCINE:Glucose tolerance impaired:0.2), (INSULIN DEGLUDEC:Glucose tolerance impaired:0.66), (INSULIN GLULISINE:Glucose tolerance impaired:0.53), (LEDIPASVIRSOFOSBUVIR:Glucose tolerance impaired:0.71), (LERCANIDIPINE:Glucose tolerance impaired:0.57), (LERCANIDIPINE HYDROCHLORIDE:Glucose tolerance impaired:0.13), (LEVALBUTEROL HYDROCHLORIDE:Glucose tolerance impaired:0.7), (LIDOCAINEPRILOCAINE:Glucose tolerance impaired:0.1), (LOMITAPIDE MESYLATE:Glucose tolerance impaired:0.46), (METAXALONE:Glucose tolerance impaired:1.09), (METHENAMINE HIPPURATE:Glucose tolerance impaired:0.2), (METHIMAZOLE:Glucose tolerance impaired:0.09), (METRONIDAZOLEMETRONIDAZOLE HYDROCHLORIDE:Glucose tolerance impaired:0.36), (MIDODRINE:Glucose tolerance impaired:0.06), (MOXIFLOXACIN HYDROCHLORIDE:Glucose tolerance impaired:0.78), (MYCOPHENOLATE MOFETILMYCOPHENOLATE MOFETIL HYDROCHLORIDE:Glucose tolerance impaired:0.35), (NALOXONE HYDROCHLORIDEOXYCODONE HYDROCHLORIDE:Glucose tolerance impaired:0.24), (NAPROXEN SODIUM:Glucose tolerance impaired:0.59), (NEBIVOLOL:Glucose tolerance impaired:0.02), (NIRAPARIB:Glucose tolerance impaired:0.02), (NORTRIPTYLINE:Glucose tolerance impaired:0.57), (OCTREOTIDE:Glucose tolerance impaired:0.07), (OXYBUTYNIN CHLORIDE:Glucose tolerance impaired:0.6), (PANTOPRAZOLE MAGNESIUM:Glucose tolerance impaired:0.78), (PATIROMER:Glucose tolerance impaired:0.11), (PAZOPANIB HYDROCHLORIDE:Glucose tolerance impaired:0.61), (PEGINTERFERON BETA-1A:Glucose tolerance impaired:0.36), (PEMETREXED DISODIUM:Glucose tolerance impaired:0.16), (PENICILLIN:Glucose tolerance impaired:0.16), (PHENOBARBITAL:Glucose tolerance impaired:0.41), (POLYETHYLENE GLYCOLS:Glucose tolerance impaired:1), (PONATINIB:Glucose tolerance impaired:0.21), (RABEPRAZOLE:Glucose tolerance impaired:0.6), (RANOLAZINE:Glucose tolerance impaired:2.17), (SCOPOLAMINE:Glucose tolerance impaired:0.41), (SULFAMETHOXAZOLE:Glucose tolerance impaired:0.2), (SUMATRIPTAN:Glucose tolerance impaired:0.65), (TERBINAFINE HYDROCHLORIDE:Glucose tolerance impaired:0.04), (TERBUTALINE SULFATE:Glucose tolerance impaired:0.17), (TETRACYCLINE:Glucose tolerance impaired:0.41), (THALIDOMIDE:Glucose tolerance impaired:0.28), (TOLTERODINE TARTRATE:Glucose tolerance impaired:0.27), (UMECLIDINIUM BROMIDEVILANTEROL TRIFENATATE:Glucose tolerance impaired:0.96), (VITAMIN A:Glucose tolerance impaired:0.01), (ZINC:Glucose tolerance impaired:0.61), (BUDESONIDE:Glycosylated haemoglobin increased:0.15), (CANDESARTAN CILEXETIL:Glycosylated haemoglobin increased:0.01), (EMPAGLIFLOZINLINAGLIPTIN:Glycosylated haemoglobin increased:0.52), (NAPROXEN:Glycosylated haemoglobin increased:0.66), (PRAVASTATIN SODIUM:Glycosylated haemoglobin increased:0.53), (RANOLAZINE:Glycosylated haemoglobin increased:0.28), (WARFARIN SODIUM:Glycosylated haemoglobin increased:0.12), (ACETAMINOPHENCODEINE PHOSPHATE:Goitre:0.15), (AMIODARONE HYDROCHLORIDE:Goitre:0.09), (ANASTROZOLE:Goitre:0.48), (BUSPIRONE HYDROCHLORIDE:Goitre:0.01), (CALCIUM CARBONATECHOLECALCIFEROL:Goitre:0.5), (CARVEDILOL:Goitre:0.51), (CORTISONE HYDROCORTISONE:Goitre:0.16), (DASATINIB:Goitre:0.06), (EVOLOCUMAB:Goitre:0.68), (FERROUS SULFATEFERROUS SULFATE, DRIED:Goitre:0.33), (IBRUTINIB:Goitre:1.13), (INSULIN HUMAN:Goitre:0.13), (METHYLPREDNISOLONE SODIUM SUCCINATE:Goitre:0.32), (NAPROXEN:Goitre:0.93), (NIFEDIPINE:Goitre:0.03), (PRAVAST ATIN:Goitre:0.16), (RABEPRAZOLE SODIUM:Goitre:0.02), (RANITIDINE HYDROCHLORIDE:Goitre:0.15), (TOPIRAMATE:Goitre:0.42), (TRAZODONE HYDROCHLORIDE:Goitre:0.42), (WARFARIN SODIUM:Goitre:0.3), (.BETA.-CAROTENE:Gout:0.01), (ABACAVIR:Gout:0.01), (ABOBOTULINUMTOXINA:Gout:0.61), (A CETYLCARNITINE HYDROCHLORIDE:Gout:0.05), (ADO-TRASTUZUMAB EMTANSINE:Gout:2.22), (ALBUTEROLIPRATROPIUM BROMIDE:Gout:0.05), (ALDESLEUKIN:Gout:0.52), (ALUMINUM HYDROXIDEMAGNESIUM OXIDEOXETHAZAINE:Gout:0.23), (AMBROXOL:Gout:0.23), (AMINO ACIDSDEXTROSEELECTROLYTES NOS:Gout:1.17), (AMINO ACIDSDEXTROSEELECTROLYTES NOSSOYBEAN OIL:Gout:0.02), (AMMONIUM CHLORIDEDEXTROMETHORPHANSODIUM CITRATE:Gout:0.44), (AMOXICILLINCLARITHROMYCINESOMEPRAZOLE:Gout:0.7), (ARGININE:Gout:0.13), (ASPIRINDIHYDROXYALUMINUM AMINOACETATE ANHYDROUSMAGNESIUM CARBONATE:Gout:0.83), (AZITHROMYCIN OR METHOTREXATE SODIUM:Gout:0.36), (BAMIFYLLINE HYDROCHLORIDE:Gout:0.32), (BELATACEPT:Gout:0.55), (BENZOCAINE:Gout:0.24), (BE NZTROPINE MESYLATE:Gout:0.39), (BEPRIDIL:Gout:0.51), (BETAMETHASONE DIPROPIONATECALCIPOTRIENE:Gout:0.55), (BETAMETHASONE DIPROPIONATECALCIPOTRIENE HYDRATE:Gout:0.19), (BLINATUMOMAB:Gout:0.67), (BOCEPREVIR:Gout:0.78), (BOTULI NUM TOXIN TYPE A:Gout:0.57), (BRINZOLAMIDETIMOLOL MALEATE:Gout:0.29), (CABOZANTINIB S-MALATE:Gout:0.54), (CALCIUM CARBONATECHOLECALCIFEROLMAGNESIUM CARBONATE:Gout:0.28), (CALCIUM PANTOTHENATECYANOCOBALAMINNIACINAMIDEPYRIDOXINE HYDROCHLORIDERIBOFLAVINTHIAMINE MONONITRATE:Gout:0.22), (CALCIUMMAGNESIUMZINC:Gout:1.22), (CANNABIS SATIVA SUBSP. INDICA TOP:Gout:0.58), (CARTEOLOL HYDROCHLORIDE:Gout:0.32), (CASPOFUNGIN:Gout:0.5), (CEFOTAXIMECEFOTAXIME SODIUM:Gout:0.38), (CERIVASTATIN SODIUM:Gout:0.19), (CEVIMELINE HYDROCHLORIDE:Gout:0.51), (CHLORAMBUCIL:Gout:0.93), (CHLORHEXIDINE:Gout:1.4), (CHONDROITIN SULFATE (CHICKEN):Gout:0.45), (CICLOPIROX:Gout:0.28), (CINACALCET:Gout:0.14), (CLODRONA TE DISODIUM:Gout:0.01), (CLOMIPRAMINE HYDROCHLORIDE:Gout:0.72), (CLOSTRIDIUM BUTYRICUM SPORES STRAIN M-55:Gout:0.69), (CLOXACILLIN SODIUM:Gout:0.01), (COLISTIN:Gout:0.35), (CYAMEMAZINE:Gout:0.51), (CYPROHEPTAD INE:Gout:1.28), (DACLIZUMAB:Gout:2.14), (DEGARELIX ACETATE:Gout:0.17), (DEXTROAMPHETAMINE:Gout:0.13), (DIOSMIN:Gout:0.44), (DIPYR ONEPROMETHAZINE HYDROCHLORIDE:Gout:0.19), (DOBUTAMINEDOBUTAMINE HYDROCHLORIDE:Gout:0.05), (DOCUSATE SODIUMSENNA LEAF:Gout:0.13), (DOLASETRON MESYLATE:Gout:1.06), (DOXYLAMINE SUCCINATE:Gout:0.62), (ELETRIPTAN HYDROBROMIDE:Gout:2.42), (EPIRUBICIN:Gout:1.3), (ERIBULIN MESYLATE:Gout:0.2), (ESTRADIOLNORETHINDRONE ACETATE:Gout:0.31), (ESTRIOL:Gout:0.33), (ETOPOSIDE PHOSPHATE:Gout:0), (ETRETINATE:Gout:0.37), (FERRIC HYDROXIDE:Gout:0.67), (FERROUS BISGLYCINATE:Gout:0.14), (FERROUS GLYCINE SULFATE:Gout:0.34), (FLUNARIZINE HYDROCHLORIDE:Gout:0.12), (FLUOROMETHOLONE:Gout:0.52), (FLURBIPROFEN:Gout: 0.38), (FORMOTEROL FUMARATEMOMETASONE FUROATE:Gout:1.62), (GLIMEPIRIDEROSIGLITAZONE MALEATE:Gout:0.64), (GLUTAMINE:Gout:0.31), (GLYCOPYRROLATEINDACATEROL MALEATE:Gout:0.99), (GLYCOSAMINOGLYCANS:Gout:0.07), (HYALURONIC ACID:Gout:0.14), (HYDROCHLOROTHIAZIDEMETOPROLOL SUCCINATE:Gout:0.69), (HYDROCODONE BITARTRATEIBUPROFEN:Gout:0.99), (HYOSCYAMINE SULFATEMETHENAMINEMETHYLENE BLUEPHENYL SALICYLATESODIUM PHOSPHATE, MONOBASIC, MONOHYDRATE:Gout:0.08), (IBRITUMOMAB TIUXETAN:Gout:0.36), (IMIPENEM:Gout:0.55), (IMIQUIMOD:Gout:0.51), (INSULIN ASPARTINSULIN DEGLUDEC:Gout:0.02), (INSULIN PORKINSULIN PURIFIED PORK:Gout:0.37), (INTERFERON:Gout:1.35), (INTERFERON ALFA-2A:Gout:1.56), (INTERFERON ALFA-2A OR INTERFERON ALFA-2B: Gout:0.11), (INTERFERON ALFA-2B:Gout:1.4), (INTERFERON GAMMA-1B:Gout:0.45), (IPRAGLIFLOZIN L-PROLINE:Gout:0.04), (IVABRADINE HYDROCHLORIDE:Gout:0.75), (LANREOTIDE ACETATE:Gout:0.81), (LAPATINIB DITOSYLATE:Gout:1.16), (LENVATINIB:Gout:0.64), (LEVOMEFOLATE CALCIUM:Gout:0.59), (LEVOSIMENDAN:Gout:0.18), (LINSIDOMINE:Gout:0.61), (LUTETI UM LU-177:Gout:0.32), (MELPERONE:Gout:0.36), (METFORMIN HYDROCHLORIDEROSIGLITAZONE MALEATE:Gout:1.01), (METFORMIN HYDROCHLORIDESAXAGLIPTIN:Gout:0.59), (METFORMINSITAGLIPTIN:Gout:0.5), (M ETHENOLONE ENANTHATE:Gout:0.68), (METRELEPTIN:Gout:0.55), (MOEXIPRIL HYDROCHLORIDE:Gout:0.77), (NALOXONE:Gout:0.5), (NEOMYCIN:Gout:0.26), (NOREPI NEPHRINE:Gout:0.81), (NOREPINEPHRINE BITARTRATE:Gout:1.28), (ORPHENADRINE:Gout:0.78), (OSELTAMIVIR:Gout:1.11), (OSPE MIFENE:Gout:0.27), (OXYBUTYNINOXYBUTYNIN CHLORIDE:Gout:0.7), (PALIPERIDONE PALMITATE:Gout:0.35), (PALONOSETRON:Gout:0.07), (PANTOPRAZOLE MAGNESIUM:Gout:3.12), (PARATHYROID HORMONE:Gout:0.16), (PASIREOTIDE:Gout:0.25), (PEGINTERFERON BETA-1A:Gout:1.55), (PEGZEREPOETIN ALFA:Gout:0.48), (PEMETREXED:Gout:1.44), (PENTOSTATIN:Gout:0.48), (PHLOROGLUCI NOL\1,3,5-TRIMETHOXYBENZENE:Gout:0.84), (PILSICAINIDE HYDROCHLORIDE:Gout:0.93), (PIRACETAM:Gout:0.15), (POLYETHYLENE GLYCOL 3350\POTASSIUM CHLORIDESODIUM BICARBONATESODIUM CHLORIDESODIUM SULFATE ANHYDROUS:Gout:0.87), (PORFIMER SODIUM:Gout:0), (POTASSIUM PHOSPHATE, MONOBASIC:Gout:0.23), (PREDNISOLONE SODIUM PHOSPHATE:Gout:0.35), (PREDNISOLONE SODIUM SUCCINATE:Gout:0.02), (PROTRIPTYLINE HYDROCHLORIDE:Gout:0.42), (PRUCALOPRIDE SUCCINATE:Gout:0.09), (QUINACRINE:Gout:0.62), (RADIATION THERAPY:Gout:1.54), (RETINOL:Gout:1.14), (RILPIVIRINE HYDROCHLORIDE:Gout:0.44), (RIZATRIPTAN:Gout:0.81), (RIZATRIPTAN BENZOATE:Gout:1.99), (RUCAPARIB CAMSYLATE:Gout:0.33), (SCOPOLAMINE HYDROBROMIDE:Gout:0.06), (SELEGILINE HYDROCHLORIDE:Gout:0.31), (SELEXIPAG:Gout:0.99), (SODIUM CITRATE:Gout:0.13), (SOFOSBUVIRVELPATASVIR:Gout:0.68), (SORBITOL:Gout:0.62), (S UFENTANIL CITRATE:Gout:0.04), (TEDUGLUTIDEWATER:Gout:0.82), (TELOTRISTAT ETHYL:Gout:0.71), (TEMSIROLIMUS:Gout:0.59), (TENELIGLIPTIN:Gout:0.22), (TENOFOVI R:Gout:0.12), (TESTOSTERONE UNDECANOATE:Gout:1.18), (TIBOLONE:Gout:0.16), (TICLOPIDINE:Gout:0.72), (TILIDINE:Gout:0.09), (TIMOLOL MALEATETRAVOPROST:Gout:0.75), (TOLTERODINE:Gout:0.98), (TRAMETINIB DIMETHYL SULFOXIDE:Gout:0.62), (TRIPTORELIN PAMOATE:Gout:0.62), (VALDECOXIB:Gout:0.21), (VANDETANIB:Gout:0.03), (VINORELBI NE TARTRATE:Gout:1.23), (VINORELBINEVINORELBINE TARTRATE:Gout:0.32), (VORAPAXAR SULFATE:Gout:0.06), (ZICONOTIDE ACETATE:Gout:0.08), (ZILEUTON:Gout:0.98), (CANDESARTAN:Gouty arthritis:0.48), (CANDESARTAN CILEXETIL:Gouty arthritis:0.21), (DEXLANSOPRAZOLE:Gouty arthritis:0.05), (EVOLOCUMAB:Gouty arthritis:0.8), (FERROUS SULFATEFERROUS SULFATE, DRIED:Gouty arthritis:0.17), (FLECAINIDE:Gouty arthritis:0.03), (GLIPIZIDE:Gouty arthritis:0.19), (OXYGEN:Gouty arthritis:0.16), (PRAVASTATIN:Gouty arthritis:0.21), (RANOLAZINE:Gouty arthritis:0.3), (SACUBITRILVALSARTAN:Gouty arthritis:0.41), (ACETAMINOPHENCODEINE PHOSPHATE:Graft versus host disease:0.9), (ACETAMINOPHENOXYCODONEOXYCODONE HYDROCHLORIDE:Graft versus host disease:0.02), (ACETAZOLAMIDE:Graft versus host disease:0.52), (AMIODARONE HYDROCHLORIDE:Graft versus host disease:0.09), (AMMONIUM GLYCYRRHIZATECYSTEINE HYDROCHLORIDEGLYCINE:Graft versus host disease:0.13), (AMPHETAMINE ASPARTATEAMPHETAMINE SULFATEDEXTROAMPHETAMINE SACCHARATEDEXTROAMPHETAMINE SULFATE:Graft versus host disease:0.55), (ANIDULAFUNGIN:Graft versus host disease:0.7),(ARMODAFINIL:Graft versus host disease:0.36), (BELATACEPT:Graft versus host disease:0.25), (BENAZEPRIL HYDROCHLORIDE:Graft versus host disease:0.12), (BENDAMUSTINE HYDROCHLORIDE:Graft versus host disease:0.38), (BISACODYL OR DOCUSATE SODIUM:Graft versus host disease:0.5), (BISOPROLOL FUMARATE:Graft versus host disease:0.08), (BRINCIDOFOVIR:Graft versus host disease:0.6), (BUPRENORPHINE HYDROCHLORIDE:Graft versus host disease:0.32), (BUPRENORPHINE HYDROCHLORIDENALOXONE HYDROCHLORIDE:Graft versus host disease:0.09), (CANAKINUMAB:Graft versus host disease:0.6), (CANDESARTAN CILEXETILHYDROCHLOROTHIAZIDE:Graft versus host disease:0.02), (CEFPIROME:Graft versus host disease:0.16), (CERTOLIZUMAB PEGOL:Graft versus host disease:1.27), (CHLORAMBUCIL:Graft versus host disease:0.7), (CLINDAMYCIN:Graft versus host disease:0.85), (CLOZAPINE:Graft versus host disease:0.33), (CYCLOPHOSPHAMIDECYTARABINEDEXAMETHASONEDOXORUBICI NMETHOTREXATEVINCRISTINE:Graft versus host disease:0.19), (CYPROHEPTADINE:Graft versus host disease:0.12), (DEXAMETHASONE ACETATE:Graft versus host disease:0.23), (DEXLANSOPRAZOLE:Graft versus host disease:0.9), (DICLOFENAC:Graft versus host disease:0.59), (DIPYRONE:Graft versus host disease:0.03), (DIVALPROEX SODIUM:Graft versus host disease:1.11), (DOXAZOSIN MESYLATE:Graft versus host disease:0.07), (DOXAZOSINDOXAZOSIN MESYLATE:Graft versus host disease:0.14), (DUTASTERIDE:Graft versus host disease:0.28), (ELETRIPTAN HYDROBROMIDE:Graft versus host disease:0.27), (ELOTUZUMAB:Graft versus host disease:0.14), (ESZOPICLONE:Graft versus host disease:1.05), (FESOTERODINE FUMARATE:Graft versus host disease:0.04), (FLECAINIDE:Graft versus host disease:0.09), (FLUCYTOSINE:Graft versus host disease:0.2), (FLUTICASONE FUROATEVILANTEROL TRIFENATATE:Graft versus host disease:0.17), (FOSFOMYCIN:Graft versus host disease:0.38), (GARENOXACIN:Graft versus host disease:0.24), (GUSPERIMUS TRIHYDROCHLORIDE:Graft versus host disease:0.4), (HYDROCHLOROTHIAZIDELISINOPRIL:Graft versus host disease:0.23), (HYDROCODONE:Graft versus host disease:0.3), (HYDROXYCHLOROQUINE DIPHOSPHATE:Graft versus host disease:0.06), (IBRITUMOMAB TIUXETAN:Graft versus host disease:1.07), (IDELALISIB:Graft versus host disease:0.55), (ILOPROST:Graft versus host disease:0.22), (IMIPRAMINE HYDROCHLORIDE:Graft versus host disease:0.03), (INTERFERON GAMMA-1B:Graft versus host disease:0.37), (IRBESARTAN:Graft versus host disease:0.03), (ISOSORBIDE:Graft versus host disease:0.09), (LACOSAMIDE:Graft versus host disease:0.27), (LOVASTATIN:Graft versus host disease:0.64), (MELOXICAM:Graft versus host disease:0.86), (METHENOLONE ENANTHATE:Graft versus host disease:0.59), (METHOTREXATEMETHOTREXATE SODIUM:Graft versus host disease:0.02), (MOGAMULIZUMAB:Graft versus host disease:0.43), (MUROMONAB-CD3:Graft versus host disease:0.04), (NAPROXEN:Graft versus host disease:1.05), (NELARABINE:Graft versus host disease:0.43), (NOREPINEPHRINE:Graft versus host disease:0.11), (OSELTAMIVIR:Graft versus host disease:0.22), (PAROXETINEPAROXETINE HYDROCHLORIDE:Graft versus host disease:0.01), (PAZOPANIB HYDROCHLORIDE:Graft versus host disease:0.01), (PAZUFLOXACIN:Graft versus host disease:0.02), (PRAMIPEXOLE DIHYDROCHLORIDE:Graft versus host disease:0.3), (PRIMIDONE:Graft versus host disease:0.4), (PROBIOTICS NOS:Graft versus host disease:0.31), (PYRAZINAMIDE:Graft versus host disease:0.66), (PYRIDOXINE HYDROCHLORIDE:Graft versus host disease:0.2), (RABEPRAZOLE:Graft versus host disease:0.1), (RANIMUSTINE:Graft versus host disease:0), (RANOLAZINE:Graft versus host disease:0.34), (SECUKINUMAB:Graft versus host disease:0.51), (SODIUM OXYBATE:Graft versus host disease:0.24), (TAZOBACTAM:Graft versus host disease:0.01), (TERBUTALINE SULFATE:Graft versus host disease:0.33), (TERIFLUNOMIDE:Graft versus host disease:0.18), (TETRACYCLINE:Graft versus host disease:0.25), (THIOGUANINE:Graft versus host disease:0.14), (TISAGENLECLEUCEL:Graft versus host disease:0.2), (VARENICLINE TARTRATE:Graft versus host disease:0.62), (VINDESINE SULFATE:Graft versus host disease:0.29), (VITAMIN B:Graft versus host disease:0.38), (AMIODARONE HYDROCHLORIDE:Granulomatosis with polyangiitis:0.04), (AMOXICILLIN:Granulomatosis with polyangiitis:0.07), (CLINDAMYCINCLINDAMYCIN PHOSPHATE:Granulomatosis with polyangiitis:0.04), (EVEROLIMUS:Granulomatosis with polyangiitis:0.11), (EVOLOCUMAB:Granulomatosis with polyangiitis:0.37), (IBRUTINIB:Granulomatosis with polyangiitis:0.28), (LERCANIDIPINE:Granulomatosis with polyangiitis:0.17), (LOVASTATIN:Granulomatosis with polyangiitis:0.28), (MAGNESIUM:Granulomatosis with polyangiitis:0.1), (NEBIVOLOL:Granulomatosis with polyangiitis:0.25), (POLYETHYLENE GLYCOLS:Granulomatosis with polyangiitis:0.05), (POTASSIUM:Granulomatosis with polyangiitis:0.38), (PRAVASTATIN:Granulomatosis with polyangiitis:0.17), (SECUKINUMAB:Granulomatosis with polyangiitis:0.35), (TOFACITINIB CITRATE:Granulomatosis with polyangiitis:0.07), (VERAPAMIL HYDROCHLORIDE:Granulomatosis with polyangiitis:0.3), (PYRAZINAMIDE:Groin abscess:0.57), (ACETAMINOPHENOXYCODONEOXYCODONE HYDROCHLORIDE:Growth hormone deficiency:0.12), (ALBUTEROL SULFATEIPRATROPIUM BROMIDE:Growth hormone deficiency:0.6), (AMIODARONE HYDROCHLORIDE:Growth hormone deficiency:0.03),(ARMODAFINIL:Growth hormone deficiency:1.18), (ATROPINE SULFATEDIPHENOXYLATE HYDROCHLORIDE:Growth hormone deficiency:0.22), (AZITHROMYCIN ANHYDROUS:Growth hormone deficiency:0.21), (BENAZEPRIL HYDROCHLORIDE:Growth hormone deficiency:0.19), (BENAZEPRILBENAZEPRIL HYDROCHLORIDE:Growth hormone deficiency:0.08), (BIFIDOBACTERIUM LONGUM SUBSP. INFANTIS:Growth hormone deficiency:0.04), (BISOPROLOL FUMARATE:Growth hormone deficiency:0.38), (CALCITONIN SALMON:Growth hormone deficiency:0.06), (CALCIUM ACETATE:Growth hormone deficiency:0.27), (CALCIUM CARBONATEVITAMIN D:Growth hormone deficiency:0.95), (CANDESARTAN:Growth hormone deficiency:0.02), (CANDESARTAN CILEXETIL:Growth hormone deficiency:0.66), (CERTOLIZUMAB PEGOL:Growth hormone deficiency:1.47), (CETIRIZINE HYDROCHLORIDEPSEUDOEPHEDRINE HYDROCHLORIDE:Growth hormone deficiency:0.03), (CLINDAMYCIN:Growth hormone deficiency:0.25), (CLINDAMYCINCLINDAMYCIN PHOSPHATE:Growth hormone deficiency:0.7), (CORTICOTROPIN:Growth hormone deficiency:0.16), (DACLIZUMAB:Growth hormone deficiency:0.33), (DASATINIB:Growth hormone deficiency:0.05), (DEVICE:Growth hormone deficiency:0.22), (DIMENHYDRINATE:Growth hormone deficiency:0.42), (DOCUSATE CALCIUM:Growth hormone deficiency:0.31), (DOXEPIN HYDROCHLORIDE:Growth hormone deficiency:0.36), (DRONABINOL:Growth hormone deficiency:0.05), (ENOXAPARIN:Growth hormone deficiency:0.79), (ESOMEPRAZOLE MAGNESIUMNAPROXEN:Growth hormone deficiency:0.14), (ESZOPICLONE:Growth hormone deficiency:1.11), (EVEROLIMUS:Growth hormone deficiency:0.44), (EVOLOCUMAB:Growth hormone deficiency:0.76), (FESOTERODINE FUMARATE:Growth hormone deficiency:0.11), (FISH OIL\OMEGA-3 FATTY ACIDS:Growth hormone deficiency:0.04), (FLUTICASONE FUROATEVILANTEROL TRIFENATATE:Growth hormone deficiency:0.04), (IBRUTINIB:Growth hormone deficiency:0.45), (IPRATROPIUM:Growth hormone deficiency:0.26), (ISOSORBIDE:Growth hormone deficiency:0.18), (LACOSAMIDE:Growth hormone deficiency:0.81), (LANREOTIDE ACETATE:Growth hormone deficiency:0.46), (LEFLUNOMIDE:Growth hormone deficiency:0.06), (LINAGLIPTIN:Growth hormone deficiency:0.19), (MECLIZINE HYDROCHLORIDE:Growth hormone deficiency:0.01), (METHYLPREDNISOLONE ACETATE:Growth hormone deficiency:0.81), (NEBIVOLOL HYDROCHLORIDE:Growth hormone deficiency:0.45), (NILOTINIB:Growth hormone deficiency:0.07), (NITROFURANTOIN:Growth hormone deficiency:0.39), (NORETHINDRONE:Growth hormone deficiency:0.64), (OXYGEN:Growth hormone deficiency:0.23), (PALIPERIDONE PALMITATE:Growth hormone deficiency:0.01), (PAROXETINE:Growth hormone deficiency:0.26), (PAROXETINEPAROXETINE HYDROCHLORIDE:Growth hormone deficiency:0.72), (PENICILLIN:Growth hormone deficiency:0.95), (PENTOSAN POLYSULFATE SODIUM:Growth hormone deficiency:0.27), (PONATINIB:Growth hormone deficiency:0.04), (PRAMIPEXOLE:Growth hormone deficiency:0.28), (PRAVASTATINPRAVASTATIN SODIUM:Growth hormone deficiency:0.15), (PRIMIDONE:Growth hormone deficiency:1.17), (PROMETHAZINEPROMETHAZINE HYDROCHLORIDE:Growth hormone deficiency:0.89), (QUETIAPINE:Growth hormone deficiency:0.88), (RABEPRAZOLE:Growth hormone deficiency:0.05), (RANOLAZINE:Growth hormone deficiency:0.77), (ROFLUMILAST:Growth hormone deficiency:0.1), (SECUKINUMAB:Growth hormone deficiency:0.49), (SOTALOL:Growth hormone deficiency:0.18), (SULFAMETHOXAZOLE:Growth hormone deficiency:0.05), (TERIFLUNOMIDE:Growth hormone deficiency:0.64), (TIOTROPIUM BROMIDE MONOHYDRATE:Growth hormone deficiency:0.85), (UMECLIDINIUM BROMIDEVILANTEROL TRIFENATATE:Growth hormone deficiency:0.32), (VARENICLINE TARTRATE:Growth hormone deficiency:1.27), (VILAZODONE HYDROCHLORIDE:Growth hormone deficiency:0.27), (VITAMIN B:Growth hormone deficiency:0.9), (ZIPRASIDONE HYDROCHLORIDEZIPRASIDONE MESYLATE:Growth hormone deficiency:0.05), (NAPROXEN:Growth of eyelashes:0.42), (BICALUTAMIDE:Haematological malignancy:0.16), (BUDESONIDE:Haematological malignancy:0.02), (CALCIUM CARBONATECHOLECALCIFEROL:Haematological malignancy:0.03), (CITALOPRAM HYDROBROMIDE:Haematological malignancy:0.24), (CLINDAMYCINCLINDAMYCIN PHOSPHATE:Haematological malignancy:0.43), (CRIZOTINIB:Haematological malignancy:0.04), (DASATINIB:Haematological malignancy:0.19), (DIGOXIN:Haematological malignancy:0.2), (EPINEPHRINE:Haematological malignancy:0.14), (IBRITUMOMAB TIUXETAN:Haematological malignancy:0.07), (IMATINIB MESYLATE:Haematological malignancy:0.2), (SACUBITRILVALSARTAN:Haematological malignancy:0.04), (TORSEMIDE:Haematological malignancy:0.25), (ACETAMINOPHENCODEINE PHOSPHATE:Haemochromatosis:0.24), (AMIODARONE:Haemochromatosis:0.12), (BORTEZ OMIB:Haemochromatosis:0.14), (BUPROPION HYDROCHLORIDE:Haemochromatosis:0.4), (DEXLANSOPRAZOLE:Haemochromatosis:0.49), (DICLOFENAC SODIUM:Haemochromatosis:0.55), (EVEROLIMUS:Haemochromatosis:0.54), (EVOLOCUMA B:Haemochromatosis:0.03), (FERROUS SULFATE:Haemochromatosis:0.31), (FERROUS SULFATEFERROUS SULFATE, DRIED:Haemochromatosis:0.09), (IRON:Haemochromatosis:0.3), (NILOTINIB:Haemochromatosis: 0.05), (OXCARBAZEPINE:Haemochromatosis:0.16), (PRAVASTATIN SODIUM:Haemochromatosis:0.37), (SACUBITRILVALSARTAN:Haemochromatosis:0.18), (T IZANIDINE HYDROCHLORIDE:Haemochromatosis:0.06), (ATRACURIUM:Haemodynamic instability:0.23), (DESFLURANE:Haemodynamic instability:0.87), (NEOSTIGMINE:Haemodynamic instability:0.54), (SUGAMMADEX:Haemodynamic instability:0.04), (ADO-TRASTUZUMAB EMTANSINE:Haemoglobin decreased:0.2), (AMIODARONE HYDROCHLORIDE:Haemoglobin decreased:0.87), (ANASTROZOLE:Haemoglobin decreased:1.21), (AZITHROMYCIN ANHYDROUS:Haemoglobin decreased:0.62), (BROMAZEPAM:Haemoglobin decreased:0.13), (CALCIUM ACETATE:Haemoglobin decreased:0.39), (CANDESARTAN:Haemoglobin decreased:0.78), (CANDESARTAN CILEXETILHYDROCHLOROTHIAZIDE:Haemoglobin decreased:0.4), (CARVEDILOL PHOSPHATE:Haemoglobin decreased:0.05), (CHOLESTYRAMINE:Haemoglobin decreased:0.42), (CINACALCET HYDROCHLORIDE:Haemoglobin decreased:0.12), (CLOTRIMAZOLE:Haemoglobin decreased:0.35), (DICYCLOMINE HYDROCHLORIDE:Haemoglobin decreased:0.03), (DIPHENHYDRAMINE:Haemoglobin decreased:0.99), (DIVALPROEX SODIUM:Haemoglobin decreased:0.36), (EDOXABAN TOSYLATE:Haemoglobin decreased:0.09), (EPINEPHRINE:Haemoglobin decreased:0.37), (ESZOPICLONE:Haemoglobin decreased:0.55), (ETORICOXIB:Haemoglobin decreased:0.42), (EVEROLIMUS:Haemoglobin decreased:0.98), (EVOLOCUMAB:Haemoglobin decreased:0.55), (EXEMESTANE:Haemoglobin decreased:0.1), (FEBUXOSTAT:Haemoglobin decreased:0.31), (FLECAINIDE:Haemoglobin decreased:0.34), (FLUTICASONE:Haemoglobin decreased:0.39), (GLICLAZIDE:Haemoglobin decreased:0.13), (GLYCOPYRRONIUMINDACATEROL:Haemoglobin decreased:0.04), (HYDROCHLOROTHIAZIDEVALSARTAN:Haemoglobin decreased:0.31), (HYDROXYCHLOROQUINE DIPHOSPHATE:Haemoglobin decreased:0.14), (HYDROXYZINE PAMOATE:Haemoglobin decreased:0.22), (IDELALISIB:Haemoglobin decreased:0.03), (INDAPAMIDEPERINDOPRIL:Haemoglobin decreased:0.31), (INDOMETHACIN:Haemoglobin decreased:0.05), (IPRATROPIUM:Haemoglobin decreased:0.52), (ISOSORBIDE:Haemoglobin decreased:0.28), (LEDIPASVIRSOFOSBUVIR:Haemoglobin decreased:0.76), (LETROZOLE:Haemoglobin decreased:0.83), (LEVALBUTEROL HYDROCHLORIDE:Haemoglobin decreased:0.3), (LITHIUM CARBONATE:Haemoglobin decreased:0.23), (NILOTINIB:Haemoglobin decreased:1.38), (OLANZAPINE:Haemoglobin decreased:0.27), (OMEGA-3-ACID ETHYL ESTERS:Haemoglobin decreased:0.09), (OSELTAMIVIR:Haemoglobin decreased:0.1), (OXYBUTYNIN CHLORIDE:Haemoglobin decreased:0.04), (PANTOPRAZOLE MAGNESIUM:Haemoglobin decreased:0.32), (PAZOPANIB HYDROCHLORIDE:Haemoglobin decreased:0.22), (PENICILLIN:Haemoglobin decreased:0.37), (PONATINIB:Haemoglobin decreased:0.27), (PREDNISOLONEPREDNISOLONE ACETATEPREDNISOLONE SODIUM SUCCINATE:Haemoglobin decreased:0.08), (PRIMIDONE:Haemoglobin decreased:0.33), (PROBIOTICS NOS:Haemoglobin decreased:0.11), (PYRIDOXINE:Haemoglobin decreased:0.33), (SECUKINUMAB:Haemoglobin decreased:0.4), (SOTALOL:Haemoglobin decreased:0.56), (SULFAMETHOXAZOLE:Haemoglobin decreased:0.25), (TOFACITINIB CITRATE:Haemoglobin decreased:0.28), (VERAPAMIL HYDROCHLORIDE:Haemoglobin decreased:0.67), (VINCRISTINE SULFATE:Haemoglobin decreased:0.26), (ACARBOSE:Haemolytic uraemic syndrome:0.18), (AGALSIDASE BETA:Haemolytic uraemic syndrome:0.17), (ALCOHOL:Haemolytic uraemic syndrome:0.4), (ALENDRONIC ACID:Haemolytic uraemic syndrome:0.07), (AMINO ACIDSDEXTROSEELECTROLYTES NOS:Haemolytic uraemic syndrome:0.25), (ANASTROZOLE:Haemolytic uraemic syndrome:0.71), (ANIDULAFUNGIN:Haemolytic uraemic syndrome:0.16),(ARMODAFINIL:Haemolytic uraemic syndrome:0.54), (BENAZEPRILBENAZEPRIL HYDROCHLORIDE:Haemolytic uraemic syndrome:0.26), (BLESELUMAB:Haemolytic uraemic syndrome:0.27), (BRINCIDOFOVIR:Haemolytic uraemic syndrome:0.16), (BUDESONIDEFORMOTEROL FUMARATE:Haemolytic uraemic syndrome:0), (BUPRENORPHINE HYDROCHLORIDE:Haemolytic uraemic syndrome:0.66), (BUSPIRONE HYDROCHLORIDE:Haemolytic uraemic syndrome:0.52), (CALCIUM CITRATE:Haemolytic uraemic syndrome:0.49), (CANAKINUMAB:Haemolytic uraemic syndrome:0.69), (CANDESARTAN CILEXETILHYDROCHLOROTHIAZIDE:Haemolytic uraemic syndrome:0.65), (CARFILZOMIB:Haemolytic uraemic syndrome:0.44), (CARISOPRODOL:Haemolytic uraemic syndrome:0.14), (CARVEDILOL PHOSPHATE:Haemolytic uraemic syndrome:0.48), (CERTOLIZUMAB PEGOL:Haemolytic uraemic syndrome:1.44), (CHOLESTYRAMINE:Haemolytic uraemic syndrome:1.23), (CLOZAPINE:Haemolytic uraemic syndrome:0.39), (DABIGATRAN:Haemolytic uraemic syndrome:0.07), (DACLIZUMAB:Haemolytic uraemic syndrome:1.04), (DANAZOL:Haemolytic uraemic syndrome:0.34), (DASATINIB:Haemolytic uraemic syndrome:0.17), (DESLORATADINE:Haemolytic uraemic syndrome:0.48), (DEXAMETHASONE ACETATE:Haemolytic uraemic syndrome:0.03), (ECONAZOLE:Haemolytic uraemic syndrome:0.43), (EDOXABAN TOSYLATE:Haemolytic uraemic syndrome:0.34), (ELETRIPTAN HYDROBROMIDE:Haemolytic uraemic syndrome:0.53), (EPLERENONE:Haemolytic uraemic syndrome:0.11), (ESZOPICLONE:Haemolytic uraemic syndrome:0.99), (ETORICOXIB:Haemolytic uraemic syndrome:0.23), (EVOLOCUMAB:Haemolytic uraemic syndrome:0.61), (FESOTERODINE FUMARATE:Haemolytic uraemic syndrome:0.02), (FLECAINIDE:Haemolytic uraemic syndrome:0.61), (FLUINDIONE:Haemolytic uraemic syndrome:0.49), (FLUTICASONE:Haemolytic uraemic syndrome:0.41), (FLUTICASONE FUROATESALMETEROL XINAFOATE:Haemolytic uraemic syndrome:0.14), (FORMOTEROL:Haemolytic uraemic syndrome:0.05), (GLIMEPIRIDE:Haemolytic uraemic syndrome:0.21), (GLYBURIDE:Haemolytic uraemic syndrome:0.09), (HYDROCHLOROTHIAZIDELOSARTAN POTASSIUM:Haemolytic uraemic syndrome:0.24), (HYDROCHLOROTHIAZIDEVALSARTAN:Haemolytic uraemic syndrome:0.02), (HYDROCORTISONE ACETATE:Haemolytic uraemic syndrome:0.19), (HYDROXYUREA:Haemolytic uraemic syndrome:0.29), (IBRUTINIB:Haemolytic uraemic syndrome:0.35), (ILOPROST:Haemolytic uraemic syndrome:0.26), (IMIPENEM:Haemolytic uraemic syndrome:0.93), (INDAPAMIDEPERINDOPRIL:Haemolytic uraemic syndrome:0.66), (INDOMETHACIN:Haemolytic uraemic syndrome:0.62), (INVESTIGATIONAL PRODUCT:Haemolytic uraemic syndrome:0.18), (IPRATROPIUM:Haemolytic uraemic syndrome:0.72), (ISOSORBIDE:Haemolytic uraemic syndrome:0.16), (LACOSAMIDE:Haemolytic uraemic syndrome:0.7), (LEDIPASVIRSOFOSBUVIR:Haemolytic uraemic syndrome:0.31), (LERCANIDIPINE HYDROCHLORIDE:Haemolytic uraemic syndrome:0.66), (LINAGLIPTIN:Haemolytic uraemic syndrome:0.26), (LISDEXAMFETAMINE DIMESYLATE:Haemolytic uraemic syndrome:0.3), (LITHIUM CARBONATE:Haemolytic uraemic syndrome:0.75), (METHIMAZOLE:Haemolytic uraemic syndrome:0.54), (METHYLPREDNISOLONE ACETATE:Haemolytic uraemic syndrome:1.07), (NAPROXEN SODIUM:Haemolytic uraemic syndrome:0.14), (NILOTINIB:Haemolytic uraemic syndrome:0.56), (NOREPINEPHRINE:Haemolytic uraemic syndrome:0.74), (OMEGA-3-ACID ETHYL ESTERS:Haemolytic uraemic syndrome:0.26), (OMEPRAZOLE SODIUM:Haemolytic uraemic syndrome:0.05), (PANTOPRAZOLE MAGNESIUM:Haemolytic uraemic syndrome:0.26), (PAROXETINE:Haemolytic uraemic syndrome:0.53), (PONATINIB:Haemolytic uraemic syndrome:0.17), (PRAMIPEXOLE DIHYDROCHLORIDE:Haemolytic uraemic syndrome:0.53), (PRAVASTATIN:Haemolytic uraemic syndrome:1.17), (PRIMIDONE:Haemolytic uraemic syndrome:0.62), (PROBIOTICS NOS:Haemolytic uraemic syndrome:0.54), (RALOXIFENE HYDROCHLORIDE:Haemolytic uraemic syndrome:0.05), (RANOLAZINE:Haemolytic uraemic syndrome:0.6), (RIZATRIPTAN BENZOATE:Haemolytic uraemic syndrome:0.33), (ROFLUMILAST:Haemolytic uraemic syndrome:0.14), (SECUKINUMAB:Haemolytic uraemic syndrome:0.63), (SODIUM OXYBATE:Haemolytic uraemic syndrome:0.43), (SODIUM PHOSPHATE, DIBASICSODIUM PHOSPHATE, MONOBASIC:Haemolytic uraemic syndrome:0.29), (SOTALOL:Haemolytic uraemic syndrome:0.71), (SUMATRIPTAN:Haemolytic uraemic syndrome:0.21), (TERBUTALINE:Haemolytic uraemic syndrome:0.27), (TINZAPARIN SODIUM:Haemolytic uraemic syndrome:0.23), (TIOTROPIUM:Haemolytic uraemic syndrome:0.19), (TOFACITINIB CITRATE:Haemolytic uraemic syndrome:0.12), (VITAMIN A:Haemolytic uraemic syndrome:0.02), (ZINC:Haemolytic uraemic syndrome:0.14), (ACETAMINOPHENBUTALBITALCAFFEINE:Haemorrhage:0.34), (ANAS TROZOLE:Haemorrhage:0.71),(ARMODAFINIL:Haemorrhage:0.34), (AZITHROMYCIN DIHYDRATE:Haemorrhage:0.34), (BECLOMETHASONE DIPROPIONATE:Haemorrhage:0.12), (BENAZEPRIL HYDROCHLORIDE:Haemorrhage:0.04), (BUSPIRONE HYDROCHLORIDE:Haemorrhage:0.3), (CALCIUM CARBONATEVITAMIN D:Haemorrhage:0.29), (CANDESARTAN CILEXETILHYDROCHLOROTHIAZIDE:Haemorrhage:0.15), (CARFILZOMIB:Haemorrhage: 0.21), (CARVEDILOL PHOSPHATE:Haemorrhage:0.18), (CEPHALEXIN:Haemorrhage:0.97), (CLINDAMYCIN: Haemorrhage:0.12), (CLINDAMYCINCLINDAMYCIN PHOSPHATE:Haemorrhage:1.14), (CLOTRIMAZOLE:Haemorrhage:0.28), (CLOZAPINE: Haemorrhage:0.6), (DASATINIB:Haemorrhage:0.33), (DESLORATADINE:Haemorrhage:0.1), (DIP YRONE:Haemorrhage:0.07), (DOXYCYCLINE:Haemorrhage:0.62), (EDOXABAN TOSYLATE:Haemorrhage:0.2), (ELETRIPTAN HYDROBROMIDE:Haemorrhage:0.26), (ENALAPRIL MALEATE:Haemorrhage:0.51), (ERYTHROMYCIN:Haemorrhage:0.2), (ESMOLOL HYDROCHLORIDE:Haemorrhage:0.53), (EVEROLIMUS:Haemorrhage:0.78), (EVOLOCUMA B:Haemorrhage:0.43), (FENOFIBRATE:Haemorrhage:0.46), (FLECAINIDE:Haemorrhage:0.3), (FLUOXETINEFLUOXETINE HYDROCHLORIDE:Haemorrhage:0.08), (FLUTICASONE:Haemorrhage:0.1), (FLUTICASON E FUROATEVILANTEROL TRIFENATATE:Haemorrhage:0.21), (HYDROCHLOROTHIAZIDELISINOPRIL:Haemorrhage: 0.56), (HYDROCHLOROTHIAZIDEVALSARTAN:Haemorrhage:0.09), (HYDROCORTISO NE SODIUM SUCCINATE:Haemorrhage:0.04), (HYDROXYCHLOROQUINE DIPHOSPHATE:Haemorrhage:0.39), (IBRUTINIB:Haemorrhage:1.29), (IMATINIB MESYLATE:Haemorrhage:0.51), (IMIPRAMINE HYDROCHLORIDE:Haemorrhage:0.14), (INFLUENZA VIRUS VACCINE:Haemorrhage:0.26), (INVESTIGATIONAL PRODUCT:Haemorrhage:0), (ISOFLURANE:Haemorrhage:0.97), (LABETALOLLABETALO L HYDROCHLORIDE:Haemorrhage:0.39), (LACOSAMIDE:Haemorrhage:0.1), (LERCANIDIPI NE:Haemorrhage:0.56), (LERCANIDIPINE HYDROCHLORIDE:Haemorrhage:0.01), (LEVALBUTEROL HYDROCHLORIDE:Haemorrhage:0.05), (LOSARTAN POTASSIUM:Haemorrhage:0.15), (MELATONIN:Haemorrhage:0.41), (METHADONE HYDROCHLORIDE:Haemorrhage:0.28), (METHYLPHENIDATE HYDROCHLORIDE:Haemorrhage:0.13), (MOXIFLOXACIN HYDROCHLORIDE:Haemorrhage:0.37), (NOREPINEPHRINE:Haemorrhage:0.02), (OXYBUT YNIN CHLORIDE:Haemorrhage:0.06), (PAROXETINEPAROXETINE HYDROCHLORIDE:Haemorrhage:0.37), (PRAMIPEXOLE DIHYDROCHLORIDE:Haemorrhage:0.32), (RANOLAZINE:Haemorrhage:0.46), (RIZATRIPT AN BENZOATE:Haemorrhage:0.26), (ROPIVACAINE:Haemorrhage:0.7), (SACUBITRILVALSA RTAN:Haemorrhage:0.15), (SODIUM OXYBATE:Haemorrhage:0.97), (SUGAMMADEX:Haemorrhage:0.64), (SUMATRIPTANSUM ATRIPTAN SUCCINATE:Haemorrhage:0.89), (THIAMYLAL SODIUM:Haemorrhage:0.18), (THIOPENTAL:Haemorrhage:0.4), (TIMOLOL:Haemorrhage:0.03), (TOCILIZUMAB:Haemorrhage:0.82), (TOPIRAMATE:Haemorrhage:2.02), (TRIAMCINOL ONE ACETONIDE:Haemorrhage:0.61), (VALGANCICLOVIR HYDROCHLORIDE:Haemorrhage:0.48), (VANCOMYCIN HYDROCHLORIDE:Haemorrhage:0.46), (VERAPAMIL HYDROCHLORIDE:Haemorrhage:0.86), (ACETAMINOPHENOXYCODONE HYDROCHLORIDE:Haemorrhage prophylaxis:0.01), (ALENDRONIC ACID:Haemorrhage prophylaxis:0.27), (AMPICILLIN:Haemorrhage prophylaxis:0.23), (ANASTROZOLE:Haemorrhage prophylaxis:0.96), (ATOVAQUONE:Haemorrhage prophylaxis:0.24), (ATRACURIUM BESYLATE:Haemorrhage prophylaxis:0.27), (BENDAMUSTINE HYDROCHLORIDE:Haemorrhage prophylaxis:0.05), (BORTEZOMIB:Haemorrhage prophylaxis:0.69), (BUPROPION HYDROCHLORIDE:Haemorrhage prophylaxis:0.26), (CALCITRIOL:Haemorrhage prophylaxis:0.62), (CALCIUM ACETATE:Haemorrhage prophylaxis:0.28), (CALCIUMVITAMIN D:Haemorrhage prophylaxis:0.29), (CARFILZOMIB:Haemorrhage prophylaxis:0.34), (CEPHALEXIN:Haemorrhage prophylaxis:1.17), (CLINDAMYCINCLINDAMYCIN PHOSPHATE:Haemorrhage prophylaxis:1.13), (CLOTRIMAZOLE:Haemorrhage prophylaxis:0.2), (COLCHICINE:Haemorrhage prophylaxis:0.18), (CORTISONEHYDROCORTISONE:Haemorrhage prophylaxis:0.49), (DASATINIB:Haemorrhage prophylaxis:0.77), (DEXLANSOPRAZOLE:Haemorrhage prophylaxis:0.01), (DEXMEDETOMIDINE HYDROCHLORIDE:Haemorrhage prophylaxis:0.84), (DIGITOXIN:Haemorrhage prophylaxis:0.11), (DOCUSATE:Haemorrhage prophylaxis:0.41), (DOXAZOSINDOXAZOSIN MESYLATE:Haemorrhage prophylaxis:0), (ENALAPRIL MALEATE:Haemorrhage prophylaxis:0.16), (EPINASTINE HYDROCHLORIDE:Haemorrhage prophylaxis:0.13), (ERGOCALCIFEROL:Haemorrhage prophylaxis:0.37), (ESMOLOL HYDROCHLORIDE:Haemorrhage prophylaxis:0.59), (EVEROLIMUS:Haemorrhage prophylaxis:0.72), (EVOLOCUMAB:Haemorrhage prophylaxis:0.3), (FLUTICASONE FUROATESALMETEROL XINAFOATE:Haemorrhage prophylaxis:0.15), (GLIPIZIDE:Haemorrhage prophylaxis:0.2), (HEPARIN CALCIUM:Haemorrhage prophylaxis:0.14), (HYDROCHLOROTHIAZIDELISINOPRIL:Haemorrhage prophylaxis:0.2), (IBRUTINIB:Haemorrhage prophylaxis:1.13), (ILOPROST:Haemorrhage prophylaxis:0.04), (IMIPENEM:Haemorrhage prophylaxis:0.17), (INDAPAMIDEPERINDOPRIL:Haemorrhage prophylaxis:0.43), (IOPAMIDOL:Haemorrhage prophylaxis:0.05), (IPRATROPIUM:Haemorrhage prophylaxis:0.27), (IRBESARTAN:Haemorrhage prophylaxis:0.3), (IRON:Haemorrhage prophylaxis:0.34), (ISOSORBIDE:Haemorrhage prophylaxis:0.22), (LEUCOVORIN CALCIUM:Haemorrhage prophylaxis:1.1), (MELOXICAM:Haemorrhage prophylaxis:0.24), (METHENOLONE ENANTHATE:Haemorrhage prophylaxis:0.11), (METRONIDAZOLEMETRONIDAZOLE HYDROCHLORIDE:Haemorrhage prophylaxis:0.09), (MOXIFLOXACIN:Haemorrhage prophylaxis:0.17), (NEOSTIGMINE:Haemorrhage prophylaxis:0.96), (NILOTINIB:Haemorrhage prophylaxis:1.95), (NITROFURANTOIN:Haemorrhage prophylaxis:0.31), (OSELTAMIVIR:Haemorrhage prophylaxis:0.31), (OXCARBAZEPINE:Haemorrhage prophylaxis:0.12), (PAZOPANIB HYDROCHLORIDE:Haemorrhage prophylaxis:0.29), (PONATINIB:Haemorrhage prophylaxis:0.41), (ROPIVACAINE HYDROCHLORIDE:Haemorrhage prophylaxis:0.14), (SECUKINUMAB:Haemorrhage prophylaxis:0.32), (SOTALOL:Haemorrhage prophylaxis:0.43), (SUCCINYLCHOLINE CHLORIDE:Haemorrhage prophylaxis:0.68), (TEMAZEPAM:Haemorrhage prophylaxis:0.04), (THIOPENTAL:Haemorrhage prophylaxis:0.29), (TINZAPARIN SODIUM:Haemorrhage prophylaxis:0.22), (TOCILIZUMAB:Haemorrhage prophylaxis:0.23), (TOPIRAMATE:Haemorrhage prophylaxis:0.34), (UROKINASE:Haemorrhage prophylaxis:0.03), (VASOPRESSIN:Haemorrhage prophylaxis:0.05), (ABACAVIR SULFATELAMIVUDINE:Haemorrhoids:0.02), (ABIRATERONE ACETATE:Haemorrhoids:0.33), (ACENOCOUMAROL:Haemorrhoids:0.26), (ACETAMINOPH ENPROPOXYPHENE HYDROCHLORIDE:Haemorrhoids:0.08), (ACETAMINOPHENPROPOXYPHENE NAPSYLATE:Haemorrhoids:0.06), (ACETAZOLAMIDE:Haemorrhoids:0.33), (ALENDRONIC ACID:Haemorrhoids:0.69), (AMANTADINE:Haemorrhoids:0.16), (AMISULPRIDE:Haemorrhoids: 0.11),(ARMODAFINIL:Haemorrhoids:0.79), (ATOVAQUONE:Haemorrhoids:0.02), (AZILS ARTAN KAMEDOXOMIL:Haemorrhoids:0.55), (BENAZEPRILBENAZEPRIL HYDROCHLORIDE:Haemorrhoids:0.46), (BLINATUMOMAB:Haemorrhoids:0.27), (BRINZO LAMIDE:Haemorrhoids:0.15), (BUDESONIDEFORMOTEROL FUMARATE:Haemorrhoids:0.1), (BUPRENORPHINE HYDROCHLORIDE:Haemorrhoids:0.09), (BUPRENORPHINE HYDROCHLORIDENALOXONE HYDROCHLORIDE:Haemorrhoids:0.35), (BUPROPION:Haemorrhoids:0.98), (CALCITONIN SALMON:Haemorrhoids:0.48), (CALCIUM ACETATE:Haemorrhoids:0.91), (CALCIUM CARBONATECHOLECALCIFEROLMAGNESIUM CARBONATE:Haemorrhoids:0.07), (CALCIUM CITRATE:Haemorrhoids:0.83), (CANAGLIFLOZINMETFORMIN HYDROCHLORIDE:Haemorrhoids:0.13), (CANDESARTAN CILEXETILHYDROCHLOROTHIAZIDE:Haemorrhoids:1.2), (CAPTOPRIL:Haemorrhoids:0.21), (CARVEDILOL PHOSPHATE:Haemorrhoids:0.54), (CHLORTHALIDONE:Haemorrhoids:0.33), (CIMETIDINE: Haemorrhoids:1), (CLOBAZAM:Haemorrhoids:0.14), (CORTICOTROPIN:Haemorrhoids:0.08), (CRIZOTINIB:Haemorrhoids:0.95), (DABRAFENIB:Haemorrhoids:0.42), (DACLIZUMAB: Haemorrhoids:0.29), (DALTEPARIN:Haemorrhoids:0.68), (DALTEPARIN SODIUM:Haemorrhoids:0.44), (DANAZOL:Haemorrhoids:0.24), (DEVICE:Haemorrhoids:0.52), (DIETARY SUPPLEMENT:Haemorrhoids:0.42), (DOLASETRON MESYLATE:Haemorrhoids:0.1), (ELETRIPTAN HYDROBROMIDE:Haemorrhoids:0.79), (ELOTUZUMAB:Haemorrhoids:0.38), (ESOMEPRAZ OLE MAGNESIUMNAPROXEN:Haemorrhoids:0.2), (EVOLOCUMAB:Haemorrhoids:0.71), (EZET IMIBESIMVASTATIN:Haemorrhoids:0.64), (FINGOLIMOD HYDROCHLORIDE:Haemorrhoids:0.25), (FLUDROCORTISONE ACETATE:Haemorrhoids:0.35), (FLUOXETINEFLUOXETINE HYDROCHLORIDE:Haemorrhoids:0.82), (FLUTICASONE FUROATESALMETEROL XINAFOATE:Haemorrhoids:0.27), (FLUVOXAMINE MALEATE:Haemorrhoids:0.01), (FORMOTEROL:Haemorrhoids:0.05), (FULVESTRANT: Haemorrhoids:0.51), (GIMERACILOTERACILTEGAFUR:Haemorrhoids:0.72), (GLYCOPYRRO NIUMINDACATEROL:Haemorrhoids:0.25), (HERBALSTURMERIC:Haemorrhoids:0.31), (H YDROCHLOROTHIAZIDEIRBESARTAN:Haemorrhoids:0.58), (HYDROCHLOROTHIAZID ETELMISARTAN:Haemorrhoids:0.18), (HYDROXYCHLOROQUINE DIPHOSPHATE:Haemorrhoids:0.27), (HYDROXYZINE PAMOATE:Haemorrhoids:1.23), (IBRITUMOMAB TIUXETAN:Haemorrhoids:0.35), (IMIPENEM:Haemorrhoids:0.04), (IMIPRAMINE HYDROCHLORIDE:Haemorrhoids:0.21), (INDAPAMIDE:Haemorrhoids:0.66), (INDAPAMID EPERINDOPRIL:Haemorrhoids:0.78), (INFLIXIMAB-DYYB:Haemorrhoids:0.23), (INOTUZUMAB OZOGAMICIN:Haemorrhoids:0.19), (INTERFERON ALFA-2A:Haemorrhoids:0.07), (IXAZOMIB:Haemorrhoids:0.35), (LAPATINIB DITOSYLATE:Haemorrhoids:0.51), (LERCANIDIPINE:Haemorrhoids:1.15), (LERCANIDIPIN E HYDROCHLORIDE:Haemorrhoids:0.65), (LEVALBUTEROL HYDROCHLORIDE:Haemorrhoids:0.91), (LIOTHYRONINE SODIUM:Haemorrhoids:0.03), (LITHIUM:Haemorrhoids:0.01), (LITHIUM CARBONATE:Haemorrhoids:0.65), (METAXALONE:Haemorrhoids:1.17), (METHENAMINE HIPPURATE:Haemorrhoids:0.52), (METHIMAZOLE:Haemorrhoids:0.37), (METHYLPREDNI SOLONE ACETATE:Haemorrhoids:0.78), (MODAFINIL:Haemorrhoids:0.6), (MOXIFLOXACIN HYDROCHLORIDE:Haemorrhoids:1.4), (MUPIROCINMUPIROCIN CALCIUM:Haemorrhoids:0.53), (NADROPARIN CALCIUM:Haemorrhoids:0.27), (NALOXONE HYDROCHLORIDEOXYCODONE HYDROCHLORIDE:Haemorrhoids:0.86), (NALOXONE HYDROCHLORIDETILIDINE HYDROCHLORIDE:Haemorrhoids:0.11), (NATEGLINIDE:Haemorrhoids:0.02), (NEBIVOLOL HYDROCHLORIDE:Haemorrhoids:0.83), (NEVIRAPINE:Haemorrhoids:0.38), (NINTEDANIB: Haemorrhoids:0.42), (OLANZAPINE:Haemorrhoids:1.33), (OLAPARIB:Haemorrhoids:0.17), (O LODATEROL HYDROCHLORIDETIOTROPIUM BROMIDE MONOHYDRATE:Haemorrhoids:0.11), (OMEGA-3-ACID ETHYL ESTERS:Haemorrhoids:0.9), (OSELTAMIVIR:Haemorrhoids:0.15), (PANTOPRAZOLE MAGNESIUM:Haemorrhoids:1.05), (PAZOPANIB HYDROCHLORIDE:Haemorrhoids:0.97), (PEGINTERFERON BETA-1A:Haemorrhoids:0.14), (PENICILLIN:Haemorrhoids:0.75), (PENTOSAN POLYSULFATE SODIUM:Haemorrhoids:0.12), (PHENOBARBITAL:Haemorrhoids:0.61), (PREDNISOLONE SODIUM PHOSPHATE:Haemorrhoids:0.05), (PRIMIDONE:Haemorrhoids:0.96), (PROPRANOLOLPRO PRANOLOL HYDROCHLORIDE:Haemorrhoids:0.27), (PYRAZINAMIDE:Haemorrhoids:0.73), (RIVASTIG MINE TARTRATE:Haemorrhoids:0.24), (ROFLUMILAST:Haemorrhoids:0.67), (ROPINIROLE: Haemorrhoids:0.26), (SACUBITRILVALSARTAN:Haemorrhoids:1.04), (TERBINAFINE HYDROCHLORIDE:Haemorrhoids:0.57), (TERBUTALINE SULFATE:Haemorrhoids:0.11), (TERIFLUNOMIDE:Haemorrhoids:0.52), (TRAMETINIB: Haemorrhoids:0.41), (TRIAMTERENE:Haemorrhoids:0.25), (UMECLIDINIUM BROMIDEVILANTEROL TRIFENATATE:Haemorrhoids:0.42), (VINORELBINE TARTRATE:Haemorrhoids:0.22), (ZINC:Haemorrhoids:0.31), (ETIMICIN SULFATE:Haemostasis:0.62), (REMIFENTANIL:Haemostasis:0.19), (SULBACTAM SODIUM:Haemostasis:0.16), (AMOXICILLIN:Hair disorder:0.38), (BISACODYL OR DOCUSATE SODIUM:Hair disorder:0.09), (CARVEDILOL PHOSPHATE:Hair disorder:0.15), (CHOLESTYRAMINE:Hair disorder:0.35), (CIPROFLOXACIN HYDROCHLORIDE:Hair disorder:0.49), (CLINDAMYCINCLINDAMYCIN PHOSPHATE:Hair disorder:0.22), (CLOTRIMAZOLE:Hair disorder:0.07), (DALFAMPRIDINE:Hair disorder:0.26), (DEVICE:Hair disorder:0.03), (DICLOFENAC:Hair disorder:0.1), (DIVALPROEX SODIUM:Hair disorder:0.33), (ELETRIPTAN HYDROBROMIDE:Hair disorder:0.57), (EPINEPHRINE:Hair disorder:0.22), (ESZOPICLONE:Hair disorder:0.41), (EVOLOCUMAB:Hair disorder:0.94), (FLUTICASONE:Hair disorder:0.04), (FLUTICASONE FUROATEVILANTEROL TRIFENATATE:Hair disorder:0.07), (HYDROCHLOROTHIAZIDELISINOPRIL:Hair disorder:0.09), (HYDROXYCHLOROQUINE DIPHOSPHATE:Hair disorder:0.14), (HYOSCYAMINE:Hair disorder:0.04), (INDOMETHACIN:Hair disorder:0.19), (MODAFINIL:Hair disorder:0.41), (NAPROXEN:Hair disorder:1.73), (OXYGEN:Hair disorder:0.22), (PROMETHAZINE HYDROCHLORIDE:Hair disorder:0.08), (PROMETHAZINEPROMETHAZINE HYDROCHLORIDE:Hair disorder:0.57), (RALOXIFENE HYDROCHLORIDE:Hair disorder:0.09), (RANOLAZINE:Hair disorder:0.33), (RIZATRIPTAN BENZOATE:Hair disorder:0.18), (SUMATRIPTANSUMATRIPTAN SUCCINATE:Hair disorder:0.33), (TAMSULOSIN HYDROCHLORIDE:Hair disorder:0.33), (TRIAMCINOLONE ACETONIDE:Hair disorder:0.33), (UMECLIDINIUM BROMIDEVILANTEROL TRIFENATATE:Hair disorder:0.23), (ACETAMINOPHENCODEINE PHOSPHATE:Hair growth abnormal:0), (ACETAMINOPHENOXYCODONE HYDROCHLORIDE:Hair growth abnormal:0.12),(ARMODAFINIL:Hair growth abnormal:0.29), (BUDESONIDE:Hair growth abnormal:0.36), (CALCIUM CARBONATECHOLECALCIFEROL:Hair growth abnormal:0.42), (CERTOLIZUMAB PEGOL:Hair growth abnormal:0.22), (DALFAMPRIDINE:Hair growth abnormal:0.29), (DICYCLOMINE HYDROCHLORIDE:Hair growth abnormal:0.07), (DIGOXIN:Hair growth abnormal:0.79), (DIVALPROEX SODIUM:Hair growth abnormal:0.24), (ELETRIPTAN HYDROBROMIDE:Hair growth abnormal:0.22), (ESTROGENS, CONJUGATEDMEDROXYPROGESTERONE ACETATE:Hair growth abnormal:0.09), (EVEROLIMUS:Hair growth abnormal:0.04), (EVOLOCUMAB:Hair growth abnormal:1.04), (IBRUTINIB:Hair growth abnormal:0.09), (INSULIN HUMAN:Hair growth abnormal:0.29), (ISOSORBIDE MONONITRATE:Hair growth abnormal:0.17), (RANOLAZINE:Hair growth abnormal:0.53), (SACUBITRILVALSARTAN:Hair growth abnormal:0.31), (TERIFLUNOMIDE:Hair growth abnormal:0.68), (TIZANIDINE HYDROCHLORIDE:Hair growth abnormal:0.01), (TRIAMCINOLONE ACETONIDE:Hair growth abnormal:0.22), (UMECLIDINIUM BROMIDEVILANTEROL TRIFENATATE:Hair growth abnormal:0.17), (.ALPHA.-TOCOPHEROL ACETATE, DL-ASCORBIC ACIDCYANOCOBALAMINFLUORIDE IONFOLIC ACIDNIACINPYRIDOXINERIBOFLAVINTHIAMINEVITAMIN AVITAMIN D:Hallucination:0.05), (ACARBOSE:Hallucination:0.18), (ACENOCOUMAROL:Hallucination: 0.35), (ACETAMINOPHENOXYCODONE HYDROCHLORIDEOXYCODONE TEREPHTHALATE:Hallucination:0.15), (ACETAMINOPHENOXYCODONEOXYCODONE HYDROCHLORIDE:Hallucination:0.64), (ALFACALCIDOL:Hallucination:0.26), (AMLODIPI NEVALSARTAN:Hallucination:0.2), (ATOMOXETINE:Hallucination:0), (ATROPINE SULFATEDIPHENOXYLATE HYDROCHLORIDE:Hallucination:0.04), (BENAZEPRILBENAZEPRIL HYDROCHLORIDE:Hallucination:0.23), (BORTEZOMIB:Hallucination:0.11), (BUPRENORP HINE HYDROCHLORIDE:Hallucination:1.53), (BUPRENORPHINENALOXONE:Hallucination:0.06), (CALCIUMMAGNESIUMZINC:Hallucination:0.16), (CANAGLIFLOZINMETFORMIN HYDROCHLORIDE:Hallucination:0.03), (CANDESARTAN:Hallucination:1.04), (CANDESAR TAN CILEXETILHYDROCHLOROTHIAZIDE:Hallucination:0.81), (CARFILZOMIB:Hallucination: 0.22), (CARISOPRODOL:Hallucination:0.81), (CARVEDILOL PHOSPHATE:Hallucination:1.08), (CHLORDIAZEPOXIDE:Hallucination:0.08), (CHLORDIAZ EPOXIDE HYDROCHLORIDECLIDINIUM BROMIDE:Hallucination:0.03), (CHOLESTYRAMINE:Hallucination:0.92), (CICLESONIDE: Hallucination:0.13), (CLINDAMYCIN:Hallucination:0.54), (CLINDAMYCINCLINDAMYCIN PHOSPHATE:Hallucination:1.17), (CLONIDINE HYDROCHLORIDE:Hallucination:0.52), (CLOTRIMAZOLE:Hallucination:0.38), (CODEINE PHOSPHATE:Hallucination:0.01), (CORTICOTROPIN:Hallucination:0.19), (CORTISONEHY DROCORTISONE:Hallucination:0.56), (DABIGATRAN:Hallucination:0.19), (DACLIZUMAB: Hallucination:0.37), (DAPAGLIFLOZIN PROPANEDIOL:Hallucination:0.32), (DASATINIB:Hallucination:0.73), (DEVICE:Hallucination: 0.55), (DIMENHYDRINATE:Hallucination:0.19), (DOXEPIN HYDROCHLORIDE:Hallucination:0.43), (EDOXABAN TOSYLATE:Hallucination:0.18), (ELETRIPTAN HYDROBROMIDE:Hallucination:1.27), (ESTROGENS:Hallucination:0.05), (EVEROLIMUS: Hallucination:0.64), (EVOLOCUMAB:Hallucination:0.97), (FENTANYL CITRATE:Hallucination:0.02), (FERROUS SULFATEFERROUS SULFATE, DRIED:Hallucination:1.48), (FEXOFENADINEPSEUDOEPHEDRINE:Hallucination:0.21), (FLUTICASONE FUROATESALMETEROL XINAFOATE:Hallucination:0.74), (FORMOTEROL:Hallucination:0.03), (FORMOTEROL FUMARATE:Hallucination:0.34), (GLYCOPYRRONIUMINDACATEROL:Hallucination:0.48), (GUANFACINE HYDROCHLORIDE:Hallucination:0.88), (HYALURONATE SODIUM:Hallucination:0.13), (HYDROCHLOROTHIAZIDEIRBESARTAN:Hallucination:0.58), (HYDROCHLOROTHIAZIDETELMISARTAN:Hallucination:0.32), (HYDROCHLOROTH IAZIDEVALSARTAN:Hallucination:1.14), (INDAPAMIDE:Hallucination:0.57), (INDAPAMI DEPERINDOPRIL:Hallucination:0.5), (INFLUENZA VIRUS VACCINE:Hallucination:0.48), (INSULIN GLULISINE:Hallucination:0.1), (LACTASE:Hallucination:0.02), (LEDIPASVIRSOFOSBUVIR: Hallucination:0.17), (LIDOCAINE HYDROCHLORIDE:Hallucination:0.5), (LOMITAPIDE MESYLATE:Hallucination:0.06), (METAMIZOLE:Hallucination:0.09), (METAXALONE:Hallucination: 1.5), (METHENAMINE HIPPURATE:Hallucination:0.73), (METHYLPREDNISOLONE ACETATE:Hallucination:0.79), (METHYLPREDNISOLONE SODIUM SUCCINATE:Hallucination:1.3), (MOMETASONE FUROATE:Hallucination:0.6), (NILOTINIB:Hallucination:0.34), (OXYMORPHONE:Hallucination: 0.18), (PANTOPRAZOLE MAGNESIUM:Hallucination:0.85), (PEGINTERFERON BETA-1A:Hallucination:0.11), (PENTOSAN POLYSULFATE SODIUM:Hallucination:0.15), (PIRIBEDIL:Hallucination:0.2), (PONATINIB:Hallucination:0.3), (PRAZOSIN HYDROCHLORIDE:Hallucination:0.21), (PREDNISOLONE ACETATE:Hallucination:0.67), (PROCYCLIDINE:Hallucination:0.19), (PYRIDOXINE:Hallucination: 0.15), (RABEPRAZOLE:Hallucination:0.6), (RIZATRIPTAN BENZOATE:Hallucination:1), (SACUBITRILVALSARTAN:Hallucination:0.32), (SAFINAMI DE:Hallucination:0.25), (SECUKINUMAB:Hallucination:0.2), (SITAGLIPTIN:Hallucination:0.04), (TERBUTALINE SULFATE:Hallucination:0.07), (TETRACYCLINE:Hallucination:0.29), (THALIDOMIDE:Hallucination: 0.26), (TIOTROPIUM:Hallucination:0.48), (TOCILIZUMAB:Hallucination:0.16), (TOC OPHEROL:Hallucination:0.07), (VALGANCICLOVIR HYDROCHLORIDE:Hallucination:0.52), (ZINC:Hallucination:0.6), (ZUCLOPENTHIXOL DECANOATE:Hallucination:0.04), (BUPRENORPHINE HYDROCHLORIDE:Hallucination, auditory:0.3), (BUPRENORPHINE HYDROCHLORIDENALOXONE HYDROCHLORIDE:Hallucination, auditory:0.12), (ENALAPRIL:Hallucination, auditory:0.02), (ESOMEPRAZOLE MAGNESIUM:Hallucination, auditory:0.1), (CALCIUM CARBONATECHOLECALCIFEROL:Head and neck cancer:0.08), (IBRUTINIB:Head and neck cancer:0.11), (LEUCOVORIN CALCIUM:Head and neck cancer:0.43), (OMEPRAZOLE MAGNESIUM:Head and neck cancer:0.08), (PANTOPRAZOLE:Head and neck cancer:0.1), (RAMIPRIL:Head and neck cancer:0.43), (REGORAFENIB:Head and neck cancer:0.17), (.ALPHA.-TOCOPHEROLRETINOL:Headache:0.12), (.ALPHA.1-PROTEINASE INHIBITOR HUMAN:Headache:2.22), (ACAMPROSATE:Headache:0.11), (ACEBUTOLOL HYDROCHLORIDE:Headache:0.89), (ACECLOFENAC:Headache:0.74), (ACETAMINOPHEN CHLORZOXAZONE:Headache:0.2), (ACETAMINOPHENMETHOCARBAMOL:Headache:00.65), (ACRIVASTINEPSEUDOEPHEDRINE HYDROCHLORIDE:Headache:0.05), (ADAPALENE:Headache:0.15), (ADENINE:Headache:0.22), (ALBUTEROLGUAIFENESIN:Headache:0.15), (ALBUTEROLIPRATROPIUM BROMIDE:Headache:0.19), (ALCOHOLALUMINUM CHLORIDE:Headache:0.02), (ALDESLEUKIN:Headache:0.66), (ALISKIREN HEMIFUMARATEHYDROCHLOROTHIAZIDE:Headache:0.21), (ALUMINUM CHLORIDE:Headache:0.04), (AMBROXOLCLENBUTEROL:Headache:0.55), (AMILORIDE HYDROCHLORIDEFUROSEMIDE:Headache:0.21), (AMINO ACIDSDEXTROSEELECTROLYTES NOSSOYBEAN OIL:Headache:0.84), (AMLODIPINE BESYLATEIRBESARTAN:Headache:0.61), (AMOXAPINE:Headache:0.94), (AMPHETAMIN E SULFATE:Headache:0.14), (AMPHETAMINEDEXTROAMPHETAMINE:Headache:0.06), (A NAKINRA:Headache:0.64), (ANTIHEMOPHILIC FACTOR, HUMAN RECOMBINANT RESIDUES 743-1636 DELETED:Headache:0.02), (APPLE CIDER VINEGAR:Headache:0.37), (ARIPIPRAZOLE LAUROXIL:Headache:0.94), (ASCORBIC ACID/CHROMIUM/SELENIUM/VANADYL SULFATE:Headache:0.52), (ASCORBIC ACIDBIOTINCALCIUM PANTOTHENATECOBALAMINFOLIC ACIDNIACINAMIDEPYRIDOXINE HYDROCHLORIDERIBOFLAVINTHIAMINE MONONITRATE:Headache:0.54), (ASCORBIC ACIDBIOTINCALCIUM PANTOTHENATECYANOCOBALAMINFOLIC ACIDNIACINAMIDEPYRIDOXINE HYDROCHLORIDERIBOFLAVINTHIAMINE MONONITRATE:Headache:0.18), (ASPARAGINASE:Headache:0.47), (ASPIRINCAFFEINE SALICYLAMIDE:Headache:0.46), (ASPIRINCLOPIDOGREL:Headache:0.11), (ASPIRINMA GNESIUM OXIDE:Headache:1.17), (ASUNAPREVIR:Headache:0.18), (ATOMOXETINE:Headache:0.04), (ATORVASTATIN CALCIUMEZETIMIBE:Headache:0.2), (ATRACURIUM:Headache:0.2), (AVOCADO OILSOYBEAN OIL:Headache:0.19), (AZITHROMYCIN OR METHOTREXATE SODIUM:Headache:0.67), (BECLOMETHASONE DIPROPIONATEFORMOTEROL:Headache:0.57), (BECLOMETHASONEFORMOTEROL: Headache:1.02), (BELATACEPT:Headache:0.47), (BENSERAZIDE HYDROCHLORIDELEVODOPA:Headache:0.31), (BENZOCAINERESORCINOL:Headache: 0.08), (BENZOIC ACIDHYOSCYAMINE SULFATEMETHENAMINEMETHYLENE BLUEPHENYL SALICYLATE:Headache:0.08), (BEPOTASTINE BESILATE:Headache:0.58), (BERAPROST:Headache:0.69), (BETAMETHASONE DIPROPIONATECALCIPOTRIENE HYDRATE:Headache:0.49), (BETAXOLOL HYDROCHLORIDE:Headache:0.5), (BIFIDOBACTERIUM LONGUMLACTOBACILLUS ACIDOPHILUS:Headache:0.09), (BISMUTH SUBCITRATE POTASSIUMMETRONIDAZOLETETRACYCLINE HYDROCHLORIDE:Headache:0.02), (BORIC ACID:Headache:0.19), (BORON:Headache:0.01), (BRIMONIDINE TARTRATEBRINZOLAMIDE:Headache:0.15), (BROMHEXINE:Headache:0.36), (CABOZAN TINIB:Headache:0.17), (CAJUPUT OILEUCALYPTUS OILPEPPERMINT OIL:Headache:0.07), (CALCIUM ASPARTATE:Headache:0.09), (CALCIUM CARBONATEERGOCALCIFEROLRETINOL:Headache:1.41), (CALCIUM CARBONATEETIDRONATE DISODIUM:Headache:0.49), (CALCIUM PANTOTHENATECYANOCOBALAMINNIACINAMIDEPYRIDOXINE HYDROCHLORIDERIBOFLAVINTHIAMINE MONONITRATE:Headache:0.87), (CALCIUMVITAMINS:Headache:0.76), (CAMOSTAT MESYLATE:Headache:0.15), (CANAGLIFLOZINMETFORMIN HYDROCHLORIDE:Headache:1.99), (CARMUSTINE:Headache:0.59), (CARTEOLOL HYDROCHLORIDE:Headache:0.36), (CARVEDILOL PHOSPHATE:Headache:3.14), (CEFOTAXIMECEFOTAXIME SODIUM:Headache:0.91), (CEFTAZIDIME SODIUM:Headache:0.1), (CEFTERAM PIVOXIL:Headache:0.09), (CEFUROXIME SODIUM:Headache:0.14), (CELIPROLOL:Headache:0.1), (CERIVASTATIN SODIUM:Headache:0.35), (CHLOROQUINE PHOSPHATE:Headache:0.32), (CHLOROTHIAZIDE:Headache:1.61), (CICLOPIROX OLAMINE:Headache:0.69), (CIFENLINE:Headache:0.02), (CISATRACURIUM:Headache:0.43), (CLADRIBINE:Headache:0.45), (CLENBUTEROL HYDROCHLORIDE:Headache:0.34), (CLIKSTAR:Headache:0.65), (CLOFARABINE:Headache: 0.07), (CLOPERASTINE:Headache:0.09), (COAGULATION FACTOR VIIA RECOMBINANT HUMAN:Headache:0.4), (COBICISTATDARUNAVIR ETHANOLATE:Headache:0.1), (CROTALIDAE POLYVALENT IMMUNE FAB (OVINE):Headache:0.22), (CYAMEMAZINE:Headache:0.72), (DEGARELIX:Headache:0.63), (DEGARELIX ACETATE:Headache:0.16), (DEXAMETHASONE ACETATE:Headache:1.8), (DIETARY SUPPLEMENTRESVERATROL:Headache:0.05), (DIFLUNISAL:Headache:0.98), (DIGITOXI N:Headache:1.15), (DILAZEP:Headache:1.42), (DIOSMIN:Headache:0.66), (DIPHTHERIA AND TETANUS TOXOIDS AND ACELLULAR PERTUSSIS VACCINE ADSORBED:Headache:0.45), (DOBUTAMINE HYDROCHLORIDE:Headache:1.09), (DROSPIRENONE:Headache:0.18), (DYDROGESTERO NE:Headache:0.34), (ECABET SODIUM:Headache:0), (EFLORNITHINE HYDROCHLORIDE:Headache:0.02), (EMPAGLIFLOZINLINAGLIPTIN:Headache:1.55), (E MTRICITABINERILPIVIRINE HYDROCHLORIDETENOFOVIR DISOPROXIL FUMARATE:Headache:0.53), (EMTRICITABINERILPIVIRINETENOFOVIR DISOPROXIL FUMARATE:Headache:0.07), (EMTRICITABINETENOFOVIR ALAFENAMIDE FUMARATE:Headache:0.09), (ENALAPRIL MALEATEHYDROCHLOROTHIAZIDE:Headache:1.05), (EPHEDRINE HYDROCHLORIDE:Headache:0.18), (ERIBULIN:Headache:0.86), (ERYTHROMYCIN LACTOBIONATE:Headache:0.15), (ESMOLOL HYDROCHLORIDE:Headache:1.01), (ESTRADIOL ACETATE:Headache:0.76), (ESTRADIOL HEMIHYDRATE:Headache:0.67), (ESTROGENSMETHYLTESTOSTERONE:Headache:0.34), (ETHINYL ESTRADIOLFERROUS FUMARATENORETHINDRONE:Headache:0.68), (ETHINYL ESTRADIOLLEVONORGESTREL ACETATE:Headache:0.19), (ETOPOSIDE PHOSPHATE:Headache:0.64), (EUCALYPTOLMENTHOLMETHYL SALICYLATETHYMOL:Headache:0.27), (FENOPROFEN CALCIUM:Headache:0.25), (FERRITIN:Headache:0.27), (FERROUS BISGLYCINATE:Headache:1.14), (FESOTERODINE:Headache:0.11), (FLUMAZENIL:Headache: 0.01), (FLUOROURACILLEUCOVORIN CALCIUMOXALIPLATIN:Headache:0.19), (FLUPHENAZINE:Headache:0.5), (FLURANDRE NOLIDE:Headache:0.02), (FLURAZEPAMFLURAZEPAM HYDROCHLORIDE:Headache:1.12), (FLUTAMIDE:Headache:0.33), (FOSINOPRIL SODIUM:Headache:0.44), (FURSULTIAMINE:Headache:0.12), (GADOBENATE DIMEGLUMINE:Headache:0.7), (GADOVERSETAMIDE:Headache:0.82), (GARLIC OIL:Headache:0.23), (GLIPIZIDEMETFORMIN HYDROCHLORIDE:Headache:0.72), (GLYCOPYRRONIUM:Headache:1.59), (GUANFACINE:Headache:1.99), (HALOBETASOL PROPIONATE:Headache:0.41), (HALOPERIDOL DECANOATE:Headache:0.61), (HEPARIN CALCIUM:Headache:1.2), (HIGH DENSITY POLYETHYLENE:Headache:0.4), (HYDROCHLOROTHIAZIDELOSARTAN:Headache:0.89), (HYDROCORTISONE PROBUTATE:Headache:0.02), (HYDROXYCHLOROQUINE DIPHOSPHATE:Headache:1.91), (HYDROXYCITRIC ACID:Headache:0.19), (HYDROXYPROPYL CELLULOSE:Headache:0.42), (HYOSCYAMINE SULFATEMETHENAMINEMETHYLENE BLUEPHENYL SALICYLATESODIUM PHOSPHATE, MONOBASIC, MONOHYDRATE:Headache:0.85), (IBRITUMOMAB TIUXETAN:Headache:0.8), (ICOSAPENT:Headache:0.42), (IDARUCIZUMAB:Headache:0.38), (IGURATIMOD:Headache:0.29), (ILOPROST:Headache:1.75), (IMIPENEM:Headache:1.47), (I NDACATEROL MALEATE:Headache:0.11), (INOTUZUMAB OZOGAMICIN:Headache:0.99), (INSULIN PORKINSULIN PURIFIED PORK:Headache:0.5), (INTERFERON:Headache:2.09), (INTERFERON ALFA-2A:Headache:1.74), (INTERFERON ALFA-2A OR INTERFERON ALFA-2B:Headache:0.16), (INTERFERON ALFA-2B:Headache:1.69), (IODINEPOTASSIUM IODIDE:Headache:0.05), (IRON POLYMALTOSE:Headache:0.65), (ISAVUCONAZONIUM SULFATE:Headache:0.18), (ISOFLURANE:Headache:0.51), (ISONIAZIDPYRAZINAMIDER IFAMPIN:Headache:0.28), (ISONIAZIDRIFAMPIN:Headache:1.02), (KALLIDINOGENASE: Headache:0.17), (KETOTIFEN:Headache:0.43), (LARONIDASE:Headache:0.11), (LATANOPR OSTTIMOLOL:Headache:0.68), (LEVOBUNOLOL HYDROCHLORIDE:Headache:0.02), (LEVOCARNITINE HYDROCHLORIDE:Headache:0.8), (LEVOLEUCOVORIN CALCIUM:Headache:1.16), (LEVOTHYROXINE SODIUMPOTASSIUM IODIDE:Headache:1.48), (LINSIDOMINE:Headache:0.07), (LULICONAZOLE:Headache:0.35), (LUMACAFTOR:Headache:0.13), (LUTETIUM LU-177:Headache:0.81), (MAGNESIUM ASPARTATEPOTASSIUM ASPARTATE:Headache:0.27), (MAGNESIUM PIDOLATE:Headache:0), (MAGNESIUM SULFATEPOTASSIUM SULFATESODIUM SULFATE:Headache:0.06), (MAPROTILINE HYDROCHLORIDE:Headache:0.39), (MELPERONE:Headache:1.23), (MEPOLIZUMAB:Headache: 1.92), (MESTRANOLNORETHINDRONE:Headache:0.72), (METFORMIN HYDROCHLORIDESAXAGLIPTIN:Headache:0.38), (METFORMINSITAGLIPTIN:Headache: 0.15), (METHENAMINE:Headache:0.88), (METHENAMINE MANDELATE:Headache:0.9), (METHENOLONE ENANTHATE:Headache:1.33), (MIGLUSTAT:Headache:1.22), (MOEXIPRIL HYDROCHLORIDE:Headache:1.26), (MOSAPRIDE CITRATE DIHYDRATE:Headache:0.15), (NAFTOPIDIL:Headache:0.3), (NEFAZODONE HYDROCHLORIDE:Headache:0.44), (NELARABINE:Headache:0.18), (NEOSTIGMINE:Headache: 0.95), (NIACINAMIDE:Headache:0.64), (NISOLDIPINE:Headache:0.32), (NOREPINEPH RINE:Headache:1.24), (NUSINERSEN:Headache:0.55), (OBETICHOLIC ACID:Headache:0), (OLODATEROL HYDROCHLORIDE:Headache:0.27), (OLOPATADINE:Headache:1.71), (OMEGA-6 FATTY ACIDS:Headache:0.4), (OPIPRAMOL:Headache:1.33), (PANITUMUMAB:Headache:0.88), (PA PAYA:Headache:0.28), (PARATHYROID HORMONE:Headache:0.58), (PEGINTERFERON ALFA-2A OR PEGINTERFERON ALFA-2B:Headache:0.08), (PEGINTERFERON ALFA-2A OR PEGINTERFERON ALFA-2B OR PEGINTERFERON BETA-1A OR PEGINTERFERON LAMBDA-1A:Headache:0.09), (PENTOSTATIN:Headache:1.39), (PERINDOPRIL ARGININE:Headache:0.03), (PERPHENAZINE:Headache:0.74), (PHENELZINE:Headache:0.49), (PHENELZINE SULFATE:Headache:0.14), (PHENPROCOUMON:Headache:1.33), (PHLOROGLUCINOL\1,3, 5-TRIMETHOXYBENZENE:Headache:0.51), (PIMECROLIMUS:Headache:1.66), (PIMOBEND AN:Headache:0.91), (PIRITRAMIDE:Headache:0.5), (POLYMYXIN:Headache:0.04), (POLYM YXIN B SULFATETRIMETHOPRIM SULFATE:Headache:0.6), (PORFIMER SODIUM:Headache:0.53), (POTASSIUM CHLORIDESODIUM CHLORIDESODIUM LACTATE:Headache:0.1), (POTASSIUM IODIDE:Headache:1.21), (POTASSIUM PHOSPHATE, MONOBASICSODIUM PHOSPHATE, DIBASIC, ANHYDROUSSODIUM PHOSPHATE, MONOBASIC, MONOHYDRATE:Headache:0.08), (PREDNISOLONE ACETATESULFACETAMIDE SODIUM:Headache:0.21), (PREGNENOLONE:Headache:0.8), (PROPIVERINE:Headache:0.12), (PROTRIPTYLINE HYDROCHLORIDE:Headache:1.65), (PRUCALOPRIDE SUCCINATE:Headache:0.45), (PSORALEN:Headache:0.02), (PYRIDOSTIGMINE:Headache:0.49), (PYRIMETHAMINE:Headache:0.59), (QUINACRINE:Headache:1.76), (RALTEGRAVIR: Headache:0.58), (RAMUCIRUMAB:Headache:1.3), (RILMENIDINE PHOSPHATE:Headache:0.74), (RILPIVIRINE HYDROCHLORIDE:Headache:0.91), (ROFECOXIB:Headache:0.86), (ROPIVACAINE:Headache: 0.18), (RUPATADINE FUMARATE:Headache:0.09), (SALSALATE:Headache:0.04), (SELEGILINE HYDROCHLORIDE:Headache:0.47), (SIBUTRAMINE HYDROCHLORIDE:Headache:1.23), (SODIUM PHOSPHATE, DIBASIC, ANHYDROUSSODIUM PHOSPHATE, DIBASIC, HEPTAHYDRATESODIUM PHOSPHATE, MONOBASIC, MONOHYDRATE:Headache:0.2), (SOLIFENACIN:Headache:0.35), (SORBITOL:Headache:0.66), (SPIRULINA:Headache:0.2), (STREPTOMYCIN SULFATE:Headache:0.17), (STREPTOMYCINSTREPTOMYCIN SULFATE:Headache:0.48), (SULFACETAMIDE SODIUM:Headache:0.17), (SULTOPRIDE HYDROCHLORIDE:Headache:0.1), (SUPLATAST TOSILATE:Headache:1.16), (TAFLUPROST:Headache:0.98), (TAMOXIFEN CITRATE:Headache:1.08), (TEDUGLUTIDE:Headache:0.91), (TEGASEROD MALEATE:Headache:0.46), (TELOTRISTAT ETHYL:Headache:1.02), (TEMOCAPRIL HYDROCHLORIDE:Headache:0.48), (TESAMORELIN:Headache:0.37), (THIAMYLAL SODIUM:Headache:0.24), (THIOCTIC ACID:Headache:0.08), (THIOPENTAL SODIUM:Headache:0.22), (TIAGABINE HYDROCHLORIDE:Headache:0.46), (TICLOPIDINE:Headache:0.52), (TIMOLOLTRAVOPR OST:Headache:0.38), (TIOPRONIN:Headache:0.2), (TIPEPIDINE HIBENZATE:Headache:0.48), (TOREMIFENE CITRATE:Headache:0.04), (TOSUFLOXACIN TOSYLATE:Headache:0.14), (TRAMETINIB DIMETHYL SULFOXIDE:Headache:0.8), (TRANYLCYPROMINE SULFATE:Headache:0.34), (TRIPTORELIN PAMOATE:Headache:0.72), (TRYPTOPHAN:Headache:0.3), (UROKINASE:Headache:0.07), (V ALDECOXIB:Headache:0.72), (VANDETANIB:Headache:0.4), (VELAGLUCERASE ALFA:Headache:0.13), (VINORELBINEVINORELBINE TARTRATE:Headache:0.63), (VONOPRAZAN FUMURATE:Headache:0.47), (YEAST:Headache:1.46), (ZALTOPROFEN:Headache:0.27), (ZIL EUTON:Headache:1.4), (ZINC SULFATEZINC SULFATE ANHYDROUS:Headache:0.36), (ZIV-AFLIBERCEPT:Headache:0.39), (CALCIUM:Heart disease congenital:0.22), (DILTIAZEM HYDROCHLORIDE:Heart disease congenital:0.18), (IBRUTINIB:Heart disease congenital:0.87), (PRAVASTATIN SODIUM:Heart disease congenital:0.02), (RANOLAZINE:Heart disease congenital:0), (AMLODIPINE BESYLATEHYDROCHLOROTHIAZIDEOLMESARTAN MEDOXOMIL:Heart rate:0.2), (BENAZEPRIL HYDROCHLORIDE:Heart rate:0.28), (CALCIUM ACETATE:Heart rate:0.23), (CARVEDILOL PHOSPHATE:Heart rate:0.45), (CEPHALEXIN:Heart rate:0.37), (CHOLESTYRAMINE:Heart rate:0.51), (CIPROFLOXACIN HYDROCHLORIDE:Heart rate:0.02), (CLINDAMYCINCLINDAMYCIN PHOSPHATE:Heart rate:0.19), (CLOTRIMAZOLE:Heart rate:0.14), (DAPAGLIFLOZIN PROPANEDIOL:Heart rate:0.24), (DASATINIB:Heart rate:0.58), (DIPHENHYDRAMINE:Heart rate:0.1), (EDOXABAN TOSYLATE:Heart rate:0.08), (ELETRIPTAN HYDROBROMIDE:Heart rate:0.32), (EPINEPHRINE:Heart rate:0.57), (ESZOPICLONE:Heart rate:0.15), (EVEROLIMUS:Heart rate:0.36), (FERROUS SULFATE:Heart rate:0.53), (FESOTERODINE FUMARATE:Heart rate:0.16), (FLUCONAZOLE:Heart rate:0.04), (GLICLAZIDE:Heart rate:0.14), (HEPARIN SODIUM:Heart rate:0.68), (HYOSCYAMINE:Heart rate:0.18), (ILOPROST:Heart rate:0.16), (INDAPAMIDE:Heart rate:0.1), (INSULIN DEGLUDEC:Heart rate:0.05), (IPRATROPIUM:Heart rate:0.21), (LABETALOLLABETALOL HYDROCHLORIDE:Heart rate:0.19), (LEDIPASVIRSOFOSBUVIR:Heart rate:0.16), (LERCANIDIPINE:Heart rate:0.59), (LERCANIDIPINE HYDROCHLORIDE:Heart rate:0.08), (LIDOCAINE HYDROCHLORIDE:Heart rate:0.02), (LISDEXAMFETAMINE DIMESYLATE:Heart rate:0.12), (LOMITAPIDE MESYLATE:Heart rate:0.18), (METAXALONE:Heart rate:0.98), (METHADONE HYDROCHLORIDE:Heart rate:0), (METHYLPREDNISOLONE SODIUM SUCCINATE:Heart rate:0.55), (MORPHINE:Heart rate:0.33), (NALOXONE HYDROCHLORIDEOXYCODONE HYDROCHLORIDE:Heart rate:0.03), (NILOTINIB:Heart rate:0.29), (NYSTATIN:Heart rate:0.1), (OXCARBAZEPINE:Heart rate:0.48), (PANTOPRAZOLE MAGNESIUM:Heart rate:0.23), (PAROXETINE:Heart rate:0.16), (PAZOPANIB HYDROCHLORIDE:Heart rate:0), (PENICILLIN:Heart rate:0.01), (PONATINIB:Heart rate:0.17), (PRAMIPEXOLE:Heart rate:0.08), (QUETIAPINE FUMARATE:Heart rate:0.17), (RISEDRONATE SODIUM:Heart rate:0.12), (RIZATRIPTAN BENZOATE:Heart rate:0.14), (SECUKINUMAB:Heart rate:0.11), (SUMATRIPTAN:Heart rate:0.04), (TERIFLUNOMIDE:Heart rate:0.36), (TIMOLOL:Heart rate:0), (UMECLIDINIUM BROMIDEVILANTEROL TRIFENATATE:Heart rate:0.38), (VARENICLINE TARTRATE:Heart rate:0.59), (ACETAMINOPHENOXYCODONEOXYCODONE HYDROCHLORIDE:Heart rate abnormal:0.07), (AMPHETAMINE ASPARTATEAMPHETAMINE SULFATEDEXTROAMPHETAMINE SACCHARATEDEXTROAMPHETAMINE SULFATE:Heart rate abnormal:0.86), (ANASTROZOLE:Heart rate abnormal:0.65),(ARMODAFINIL:Heart rate abnormal:0.77), (ATROPINE SULFATEDIPHENOXYLATE HYDROCHLORIDE:Heart rate abnormal:0.25), (BISACODYL OR DOCUSATE SODIUM:Heart rate abnormal:0.75), (BUPROPION:Heart rate abnormal:0.45), (CALCIUM CARBONATECHOLECALCIFEROL:Heart rate abnormal:0.68), (CANDESARTAN CILEXETILHYDROCHLOROTHIAZIDE:Heart rate abnormal:0.47), (CAPTOPRIL:Heart rate abnormal:0.13), (CARFILZOMIB:Heart rate abnormal:0.02), (CARVEDILOL PHOSPHATE:Heart rate abnormal:0.74), (CORTISONEHYDROCORTISONE:Heart rate abnormal:0.18), (DALFAMPRIDINE:Heart rate abnormal:0.41), (DASATINIB:Heart rate abnormal:0.26), (DEVICE:Heart rate abnormal:0.96), (DIPHENHYDRAMINE:Heart rate abnormal:0.54), (DOXAZOSINDOXAZOSIN MESYLATE:Heart rate abnormal:0.72), (ELETRIPTAN HYDROBROMIDE:Heart rate abnormal:0.44), (EMPAGLIFLOZINLINAGLIPTIN:Heart rate abnormal:0.39), (ENOXAPARIN:Heart rate abnormal:0.82), (EPINEPHRINE:Heart rate abnormal:0.64), (EVEROLIMUS:Heart rate abnormal:0.42), (EZETIMIBESIMVASTATIN:Heart rate abnormal:0.4), (FLUTICASONE:Heart rate abnormal:0.38), (FLUTICASONE FUROATEVILANTEROL TRIFENATATE:Heart rate abnormal:0.2), (HERBALSTURMERIC:Heart rate abnormal:0.06), (HYDROCHLOROTHIAZIDETELMISARTAN:Heart rate abnormal:0.02), (HYDROMORPHONE:Heart rate abnormal:0.12), (HYOSCYAMINE:Heart rate abnormal:0.32), (ILOPROST:Heart rate abnormal:0.28), (INDOMETHACIN:Heart rate abnormal:0.15), (INSULIN DEGLUDEC:Heart rate abnormal:0.14), (INSULIN GLULISINE:Heart rate abnormal:0.12), (LENALIDOMIDE:Heart rate abnormal:0.37), (LERCANIDIPINE:Heart rate abnormal:0.43), (LITHIUM CARBONATE:Heart rate abnormal:0.51), (LOMITAPIDE MESYLATE:Heart rate abnormal:0.15), (MECLIZINE HYDROCHLORIDE:Heart rate abnormal:0.03), (MELOXICAM:Heart rate abnormal:1.45), (METHADONE HYDROCHLORIDE:Heart rate abnormal:0.41), (METHENAMINE HIPPURATE:Heart rate abnormal:0.24), (METHYLPREDNISOLONE ACETATE:Heart rate abnormal:0.08), (METHYLPREDNISOLONE SODIUM SUCCINATE:Heart rate abnormal:0.52), (MIDODRINE:Heart rate abnormal:0.28), (NALOXONE HYDROCHLORIDEOXYCODONE HYDROCHLORIDE:Heart rate abnormal:0.11), (PANTOPRAZOLE MAGNESIUM:Heart rate abnormal:0.49), (PAROXETINEPAROXETINE HYDROCHLORIDE:Heart rate abnormal:0.76), (PHENOBARBITAL:Heart rate abnormal:0.21), (POLYETHYLENE GLYCOLS:Heart rate abnormal:0.73), (PYRIDOXINE HYDROCHLORIDE:Heart rate abnormal:0.14), (RIZATRIPTAN BENZOATE:Heart rate abnormal:0.03), (SCOPOLAMINE:Heart rate abnormal:0.2), (SODIUM OXYBATE:Heart rate abnormal:0.65), (SUMATRIPTANSUMATRIPTAN SUCCINATE:Heart rate abnormal:0.24), (TERIFLUNOMIDE:Heart rate abnormal:0.58), (TIOTROPIUM:Heart rate abnormal:0.02), (TOCILIZUMAB:Heart rate abnormal:0.1), (UMECLIDINIUM BROMIDEVILANTEROL TRIFENATATE:Heart rate abnormal:0.45), (VILAZODONE HYDROCHLORIDE:Heart rate abnormal:0.03), (ZINC:Heart rate abnormal:0.12), (CITALOPRAM HYDROBROMIDE:Heart rate decreased:0.1), (EVOLOCUMAB:Heart rate decreased:0.4), (IBRUTINIB:Heart rate decreased:0.94), (ILOPROST:Heart rate decreased:0.31), (IRON:Heart rate decreased:0.12), (MELOXICAM:Heart rate decreased:0.11), (RANOLAZINE:Heart rate decreased:0.62), (.ALPHA.1-PROTEINASE INHIBITOR HUMAN:Heart rate increased:0.18), (ACARBOSE:Heart rate increased:0.22), (AMLODIPINE BESYLATEHYDROCHLOROTHIAZIDEOLMESARTAN MEDOXOMIL:Heart rate increased:0.37), (BISACODYL OR DOCUSATE SODIUM:Heart rate increased:0.95), (BROMAZEPAM:Heart rate increased:0.18), (BUPRENORPHINE HYDROCHLORIDE:Heart rate increased:0.17), (BUPRENORPHINE HYDROCHLORIDENALOXONE HYDROCHLORIDE:Heart rate increased:0.37), (CALCIUM ACETATE:Heart rate increased:0.75), (CALCIUMMAGNESIUMZINC:Heart rate increased:0.05), (CANAGLIFLOZINMETFORMIN HYDROCHLORIDE:Heart rate increased:0.43), (CARVEDILOL PHOSPHATE:Heart rate increased:1.18), (CETIRIZINE HYDROCHLORIDEPSEUDOEPHEDRINE HYDROCHLORIDE:Heart rate increased:0.28), (CIMETIDINE:Heart rate increased:0.29), (CLINDAMYCIN:Heart rate increased:0.07), (CLOTRIMAZOLE:Heart rate increased:0.4), (DABIGATRAN:Heart rate increased:0.34), (DACLIZUMAB:Heart rate increased:0.39), (DARBEPOETIN ALFA:Heart rate increased:0.01), (DASATINIB:Heart rate increased:0.9), (DEFLAZACORT:Heart rate increased:0), (DEVICE:Heart rate increased:1.07), (DIMENHYDRINATE:Heart rate increased:0.24), (DOCUSATE CALCIUM:Heart rate increased:0.67), (DOXEPIN:Heart rate increased:0.14), (DRONABINOL:Heart rate increased:0.29), (EMPAGLIFLOZINLINAGLIPTIN:Heart rate increased:0.66), (ESOMEPRAZOLE MAGNESIUMNAPROXEN:Heart rate increased:0.24), (EVEROLIMUS:Heart rate increased:0.6), (EZETIMIBESIMVASTATIN:Heart rate increased:0.81), (FLUDROCORTISONE ACETATE:Heart rate increased:0.08), (FLUTICASONE FUROATE:Heart rate increased:0.49), (GLYCOPYRRONIUMINDACATEROL:Heart rate increased:0.33), (HYDROCHLOROTHIAZIDEIRBESARTAN:Heart rate increased:0.48), (HYDROCHLOROTHIAZIDETELMISARTAN:Heart rate increased:0.09), (HYDROXYCHLOROQUINE DIPHOSPHATE:Heart rate increased:0.09), (ILOPROST:Heart rate increased:0.39), (IMATINIB MESYLATE:Heart rate increased:0.02), (INDAPAMIDEPERINDOPRIL:Heart rate increased:0.13), (INSULIN GLULISINE:Heart rate increased:0.23), (LENALIDOMIDE:Heart rate increased:0.83), (LERCANIDIPINE:Heart rate increased:0.68), (LERCANIDIPINE HYDROCHLORIDE:Heart rate increased:0.07), (LEVOMEFOLATE CALCIUM:Heart rate increased:0.08), (LIDOCAINEPRILOCAINE:Heart rate increased:0.05), (LOMITAPIDE MESYLATE:Heart rate increased:0.45), (MEPOLIZUMAB:Heart rate increased:0.1), (METHYLPREDNISOLONE ACETATE:Heart rate increased:0.63), (METHYLPREDNISOLONE SODIUM SUCCINATE:Heart rate increased:0.95), (MUPIROCIN:Heart rate increased:0.03), (NALOXONE HYDROCHLORIDEOXYCODONE HYDROCHLORIDE:Heart rate increased:0.55), (NATEGLINIDE:Heart rate increased:0.1), (NILOTINIB:Heart rate increased:0.66), (NORETHINDRONE:Heart rate increased:0.38), (OLODATEROL HYDROCHLORIDETIOTROPIUM BROMIDE MONOHYDRATE:Heart rate increased:0.45), (PANTOPRAZOLE MAGNESIUM:Heart rate increased:1.25), (PATIROMER:Heart rate increased:0.07), (PERINDOPRIL:Heart rate increased:0.25), (PHENOBARBITAL:Heart rate increased:0.72), (PRAZOSIN:Heart rate increased:0.01), (PRAZOSIN HYDROCHLORIDE:Heart rate increased:0.13), (PREDNISOLONE ACETATE:Heart rate increased:0.4), (RABEPRAZOLE:Heart rate increased:0.47), (RIZATRIPTAN BENZOATE:Heart rate increased:1.01), (SECUKINUMAB:Heart rate increased:0.39), (TERBUTALINE SULFATE:Heart rate increased:0.74), (TETRACYCLINE:Heart rate increased:0.85), (THYROID, PORCINETHYROID, UNSPECIFIED:Heart rate increased:0.02), (TIOTROPIUM:Heart rate increased:0.23), (TOCILIZUMAB:Heart rate increased:0.07), (UMECLIDINIUM BROMIDEVILANTEROL TRIFENATATE:Heart rate increased:1.41), (ZAFIRLUKAST:Heart rate increased:0.11), (ZINC:Heart rate increased:0.41), (ACARBOSE:Heart rate irregular:0.52), (ACETAMINOPHENOXYCODONEOXYCODONE HYDROCHLORIDE:Heart rate irregular:0.6), (ACETAMINOPHENTRAMADOL:Heart rate irregular:0), (ALBIGLUTIDE:Heart rate irregular:0.69), (ALENDRONIC ACID:Heart rate irregular:0.22), (AMISULPRIDE:Heart rate irregular:0), (AMLODIPINE BESYLATEHYDROCHLOROTHIAZIDEOLMESARTAN MEDOXOMIL:Heart rate irregular:0.37), (AMLODIPINEVALSARTAN:Heart rate irregular:0.18), (BISACODYL:Heart rate irregular:0.6), (BROMAZEPAM:Heart rate irregular:0.28), (CALCIUM ACETATE:Heart rate irregular:0.85), (CALCIUMMAGNESIUM:Heart rate irregular:0.06), (CANAKINUMAB:Heart rate irregular:0.04), (CARFILZOMIB:Heart rate irregular:0.5), (CETIRIZINE HYDROCHLORIDEPSEUDOEPHEDRINE HYDROCHLORIDE:Heart rate irregular:0.53), (COD LIVER OIL:Heart rate irregular:0.33), (DACLIZUMAB:Heart rate irregular:0.83), (DAPAGLIFLOZIN:Heart rate irregular:0.01), (DAPAGLIFLOZIN PROPANEDIOL:Heart rate irregular:0.91), (DARBEPOETIN ALFA:Heart rate irregular:0.28), (DASATINIB:Heart rate irregular:1.29), (DIMENHYDRINATE:Heart rate irregular:0.41), (DORZOLAMIDE HYDROCHLORIDE:Heart rate irregular:0.23), (DOXEPIN HYDROCHLORIDE:Heart rate irregular:0.51), (DRONABINOL:Heart rate irregular:0.68), (DULAGLUTIDE:Heart rate irregular:0.25), (DUTASTERIDETAMSULOSIN HYDROCHLORIDE:Heart rate irregular:0.04), (EMPAGLIFLOZINLINAGLIPTIN:Heart rate irregular:0.87), (ENOXAPARIN:Heart rate irregular:1.22), (ERYTHROMYCIN:Heart rate irregular:0.2), (FEXOFENADINEPSEUDOEPHEDRINE:Heart rate irregular:0.11), (FLUDROCORTISONE ACETATE:Heart rate irregular:0.09), (FLUOCINONIDE:Heart rate irregular:0.39), (FLUTICASONE FUROATESALMETEROL XINAFOATE:Heart rate irregular:0.53), (FORMOTEROL FUMARATEMOMETASONE FUROATE:Heart rate irregular:0.81), (HYDROCODONE BITARTRATEIBUPROFEN:Heart rate irregular:0.04), (ILOPROST:Heart rate irregular:0.54), (IMATINIB MESYLATE:Heart rate irregular:0.49), (IMIPRAMINE HYDROCHLORIDE:Heart rate irregular:0.79), (INDACATEROL:Heart rate irregular:0.1), (INDAPAMIDEPERINDOPRIL:Heart rate irregular:0.13), (INFLUENZA VIRUS VACCINE:Heart rate irregular:0.39), (INSULIN GLULISINE:Heart rate irregular:0.36), (KETOROLACKETOROLAC TROMETHAMINE:Heart rate irregular:0.01), (LEDIPASVIRSOFOSBUVIR:Heart rate irregular:1.04), (LERCANIDIPINE:Heart rate irregular:0.62), (LERCANIDIPINE HYDROCHLORIDE:Heart rate irregular:0.48), (LOMITAPIDE MESYLATE:Heart rate irregular:0.81), (METFORMIN HYDROCHLORIDEROSIGLITAZONE MALEATE:Heart rate irregular:0.29), (METHADONE HYDROCHLORIDE:Heart rate irregular:0.48), (METHENAMINE HIPPURATE:Heart rate irregular:0.82), (METHYLPREDNISOLONE ACETATE:Heart rate irregular:1), (MOXIFLOXACIN HYDROCHLORIDE:Heart rate irregular:1.41), (NALOXONE HYDROCHLORIDEOXYCODONE HYDROCHLORIDE:Heart rate irregular:0.65), (NEBIVOLOL:Heart rate irregular:0.64), (NILOTINIB:Heart rate irregular:0.79), (OXYBUTYNINOXYBUTYNIN CHLORIDE:Heart rate irregular:0.05), (PANTOPRAZOLE MAGNESIUM:Heart rate irregular:1.66), (PAROXETINEPAROXETINE HYDROCHLORIDE:Heart rate irregular:1.17), (PEGINTERFERON BETA-1A:Heart rate irregular:0.9), (PEMETREXED DISODIUM:Heart rate irregular:0.22), (PENICILLIN:Heart rate irregular:0.6), (PENTOSAN POLYSULFATE SODIUM:Heart rate irregular:0.18), (PIMECROLIMUS:Heart rate irregular:0.26), (PIOGLITAZONE:Heart rate irregular:0.08), (PONATINIB:Heart rate irregular:0.32), (PREDNISOLONE ACETATE:Heart rate irregular:1.11), (QUINAPRIL:Heart rate irregular:0.12), (RABEPRAZOLE:Heart rate irregular:0.79), (RIZATRIPTAN:Heart rate irregular:0.05), (RIZATRIPTAN BENZOATE:Heart rate irregular:0.97), (SCOPOLAMINE:Heart rate irregular:0.46), (SECUKINUMAB:Heart rate irregular:1.28), (SULFAMETHOXAZOLE:Heart rate irregular:0.01), (TERBINAFINE HYDROCHLORIDE:Heart rate irregular:0.52), (TERBUTALINE SULFATE:Heart rate irregular:0.8), (TETRACYCLINE:Heart rate irregular:1.06), (THALIDOMIDE:Heart rate irregular:0.04), (THYROID, PORCINETHYROID, UNSPECIFIED:Heart rate irregular:0.06), (TOCOPHEROL:Heart rate irregular:0.07), (UMECLIDINIUM BROMIDE:Heart rate irregular:0.34), (VILAZODONE HYDROCHLORIDE:Heart rate irregular:0.85), (VITAMIN A:Heart rate irregular:0.54), (ZILEUTON:Heart rate irregular:0.17), (ACETAMINOPHENCODEINE PHOSPHATE:Heart transplant:0.24), (BUPROPION HYDROCHLORIDE:Heart transplant:0.08), (CANDESARTAN CILEXETILHYDROCHLOROTHIAZIDE:Heart transplant:0.35), (CLINDAMYCINCLINDAMYCIN PHOSPHATE:Heart transplant:0.55), (DEXLANSOPRAZOLE:Heart transplant:0.32), (DIPHENHYDRAMINE:Heart transplant:0.32), (ENOXAPARIN:Heart transplant:0.86), (EVOLOCUMAB:Heart transplant:0.04), (FLECAINIDE:Heart transplant:0.23), (IBRUTINIB:Heart transplant:0.8), (ILOPROST:Heart transplant:0.07), (ISOSORBIDE:Heart transplant:0.13), (LEDIPASVIRSOFOSBUVIR:Heart transplant:0.07), (LERCANIDIPINE HYDROCHLORIDE:Heart transplant:0.03), (NAPROXEN:Heart transplant:0.2), (NILOTINIB:Heart transplant:0.03), (OXYGEN:Heart transplant:0.47), (RANOLAZINE:Heart transplant:0.74), (SACUBITRILVALSARTAN:Heart transplant:0.16), (TOPIRAMATE:Heart transplant:0.39), (VENLAFAXINE HYDROCHLORIDE:Heart transplant:0.08), (BUPROPION HYDROCHLORIDE:Heart valve replacement:0.08), (DEVICE:Heart valve replacement:0.18), (DEXLANSOPRAZOLE:Heart valve replacement:0.41), (DICLOFENAC:Heart valve replacement:0.14), (ENALAPRIL:Heart valve replacement:0.9), (EVOLOCUMAB:Heart valve replacement:0.91), (HYDROXYCHLOROQUINE SULFATE:Heart valve replacement:0.23), (IBRUTINIB:Heart valve replacement:0.45), (ISOSORBIDE MONONITRATE:Heart valve replacement:0.04), (LEDIPASVIRSOFOSBUVIR:Heart valve replacement:0.06), (LOVASTATIN:Heart valve replacement:0.23), (NEBIVOLOL:Heart valve replacement:0.61), (POLYETHYLENE GLYCOLS:Heart valve replacement:0.04), (SACUBITRILVALSARTAN:Heart valve replacement:0.06), (TERIFLUNOMIDE:Heart valve replacement:0.03), (AMIODARONE:Helicobacter infection:0.41), (AMIODARONE HYDROCHLORIDE:Helicobacter infection:0.36), (BROMAZEPAM:Helicobacter infection:0.12), (BUMETANIDE:Helicobacter infection:0.42), (CANDESARTAN CILEXETILHYDROCHLOROTHIAZIDE:Helicobacter infection:0.15), (DASATINIB:Helicobacter infection:0.08), (DICLOFENAC SODIUM:Helicobacter infection:0.48), (DIVALPROEX SODIUM:Helicobacter infection:0.2), (DOCUSATE SODIUM:Helicobacterinfection:0.3), (ENOXAPARIN:Helicobacter infection:0.29), (ENOXAPARIN SODIUM:Helicobacter infection:0.15), (ERGOCALCIFEROL:Helicobacter infection:0.11), (ERYTHROPOIETIN:Helicobacter infection:0.21), (EVOLOCUMAB:Helicobacter infection:0.38), (HYDROXYCHLOROQUINE SULFATE:Helicobacter infection:0.19), (INDAPAMIDEPERINDOPRIL:Helicobacter infection:0.04), (INSULIN LISPRO:Helicobacter infection:0.47), (NAPROXEN SODIUM:Helicobacter infection:0.14), (POLYETHYLENE GLYCOLS:Helicobacter infection:0.1), (PRAMIPEXOLE:Helicobacter infection:0.02), (PRAVASTATIN:Helicobacter infection:0.39), (RANOLAZINE:Helicobacter infection:0.04), (SACUBITRILVALSARTAN:Helicobacter infection:0.55), (ACETAMINOPHENCODEINE PHOSPHATE:Hepatic cancer:0.72), (ADO-TRASTUZUMAB EMTANSINE:Hepatic cancer:0.73), (ALBUTEROL SULFATEIPRATROPIUM BROMIDE:Hepatic cancer:0.28), (AMIODARONE HYDROCHLORIDE:Hepatic cancer:0.66), (AMPHETAMINE ASPARTATEAMPHETAMINE SULFATEDEXTROAMPHETAMINE SACCHARATEDEXTROAMPHETAMINE SULFATE:Hepatic cancer:0.06), (BISACODYL OR DOCUSATE SODIUM:Hepatic cancer:0.33), (BORTEZOMIB:Hepatic cancer:0.27), (BUDESONIDE:Hepatic cancer:0.93), (BUMETANIDE:Hepatic cancer:0.91), (CALCIUM CARBONATECHOLECALCIFEROL:Hepatic cancer:0.81), (CANDESARTAN:Hepatic cancer:0.71), (CANDESARTAN CILEXETIL:Hepatic cancer:0.58), (CANDESARTAN CILEXETILHYDROCHLOROTHIAZIDE:Hepatic cancer:0.22), (CERTOLIZUMAB PEGOL:Hepatic cancer:0.62), (CORTISONEHYDROCORTISONE:Hepatic cancer:0.46), (DALTEPARIN SODIUM:Hepatic cancer:0.06), (DEVICE:Hepatic cancer:0.26), (DULOXETINE:Hepatic cancer:0.18), (ERIBULIN:Hepatic cancer:0.06), (EVOLOCUMAB:Hepatic cancer:0.43), (EZETIMIBE:Hepatic cancer:0.39), (FLUTICASONE:Hepatic cancer:0.06), (IBRUTINIB:Hepatic cancer:1.35), (LERCANIDIPINE:Hepatic cancer:0.04), (MELATONIN:Hepatic cancer:0.26), (MOXIFLOXACIN HYDROCHLORIDE:Hepatic cancer:0.42), (NEBIVOLOL HYDROCHLORIDE:Hepatic cancer:0.13), (NILOTINIB:Hepatic cancer:1.28), (NIRAPARIB:Hepatic cancer:0.04), (NITROFURANTOIN:Hepatic cancer:0.62), (OLAPARIB:Hepatic cancer:0.22), (OMEGA-3-ACID ETHYL ESTERS:Hepatic cancer:0.35), (OXCARBAZEPINE:Hepatic cancer:0.15), (OXYGEN:Hepatic cancer:0.4), (PANTOPRAZOLE MAGNESIUM:Hepatic cancer:0.25), (PEMETREXED:Hepatic cancer:0.1), (PRIMIDONE:Hepatic cancer:0.45), (QUETIAPINE FUMARATE:Hepatic cancer:0.03), (RADIATION THERAPY:Hepatic cancer:0.06), (ROFLUMILAST:Hepatic cancer:0.01), (SACUBITRILVALSARTAN:Hepatic cancer:0.12), (SODIUM BICARBONATE:Hepatic cancer:0.28), (SOTALOL:Hepatic cancer:0.52), (TINZAPARIN SODIUM:Hepatic cancer:0.19), (TIZANIDINE HYDROCHLORIDE:Hepatic cancer:0.14), (UMECLIDINIUM BROMIDEVILANTEROL TRIFENATATE:Hepatic cancer:0.07), (VEMURAFENIB:Hepatic cancer:0.22), (VITAMIN B:Hepatic cancer:0.19), (ABIRATERONE ACETATE:Hepatic cirrhosis:0.13), (ACETAMINOPHENBUTALBITALCAFFEINE:Hepatic cirrhosis:0.44), (ACETAMINOPHENCODEINE PHOSPHATE:Hepatic cirrhosis:1.34), (ACTIVATED CHARCOAL:Hepatic cirrhosis:0.17), (ALCOHOL:Hepatic cirrhosis:0.09), (AMOXICILLINCLAVULANATE POTASSIUM:Hepatic cirrhosis:0.59), (AMOXICILLINCLAVULANIC ACID:Hepatic cirrhosis:0.19), (AMPHETAMINE ASPARTATEAMPHETAMINE SULFATEDEXTROAMPHETAMINE SACCHARATEDEXTROAMPHETAMINE SULFATE:Hepatic cirrhosis:0.2),(ARMODAFINIL:Hepatic cirrhosis:0.23), (BERAPROST SODIUM:Hepatic cirrhosis:0.83), (BIFIDOBACTERIUM SPP.:Hepatic cirrhosis:0.49), (BUDESONIDEFORMOTEROL FUMARATE:Hepatic cirrhosis:0.1), (CALCIUM ACETATE:Hepatic cirrhosis:0.54), (CANAGLIFLOZINMETFORMIN HYDROCHLORIDE:Hepatic cirrhosis:0.43), (CARVEDILOL PHOSPHATE:Hepatic cirrhosis:0.57), (CERTOLIZUMAB PEGOL:Hepatic cirrhosis:1.29), (CLINDAMYCINCLINDAMYCIN PHOSPHATE:Hepatic cirrhosis:1.04), (CLOZAPINE:Hepatic cirrhosis:0.67), (DACLIZUMAB:Hepatic cirrhosis:0.19), (DALFAMPRIDINE:Hepatic cirrhosis:0.16), (DAPAGLIFLOZIN PROPANEDIOL:Hepatic cirrhosis:0.03), (DASATINIB:Hepatic cirrhosis:1), (DEVICE:Hepatic cirrhosis:0.47), (DOCUSATE CALCIUM:Hepatic cirrhosis:0.22), (DOFETILIDE:Hepatic cirrhosis:0.19), (ELETRIPTAN HYDROBROMIDE:Hepatic cirrhosis:0.3), (EPINEPHRINE:Hepatic cirrhosis:0.72), (ERYTHROMYCIN:Hepatic cirrhosis:0.08), (ETRETINATE:Hepatic cirrhosis:0.27), (EVEROLIMUS:Hepatic cirrhosis:1.51), (EVOLOCUMAB:Hepatic cirrhosis:1.29), (FERROUS FUMARATE:Hepatic cirrhosis:0.11), (FESOTERODINE FUMARATE:Hepatic cirrhosis:0.4), (FLECAINIDE:Hepatic cirrhosis:0.83), (FLUDROCORTISONE ACETATE:Hepatic cirrhosis:0.01), (FLUTICASONE FUROATEVILANTEROL TRIFENATATE:Hepatic cirrhosis:0.91), (FORMOTEROL FUMARATE:Hepatic cirrhosis:0.02), (FUSIDIC ACID:Hepatic cirrhosis:0.22), (HYDROCHLOROTHIAZIDEIRBESARTAN:Hepatic cirrhosis:0.56), (HYDROCHLOROTHIAZIDELISINOPRIL:Hepatic cirrhosis:0.72), (HYDROXYZINE PAMOATE:Hepatic cirrhosis:0.59), (IBRUTINIB:Hepatic cirrhosis:1.67), (IDELALISIB:Hepatic cirrhosis:0.12), (INDOMETHACIN:Hepatic cirrhosis:0.06), (LERCANIDIPINE:Hepatic cirrhosis:1.44), (LIDOCAINE HYDROCHLORIDE:Hepatic cirrhosis:0.34), (LIDOCAINEPRILOCAINE:Hepatic cirrhosis:0.21), (LISDEXAMFETAMINE DIMESYLATE:Hepatic cirrhosis:0.11), (LOMITAPIDE MESYLATE:Hepatic cirrhosis:0.04), (LOVASTATIN:Hepatic cirrhosis:1), (METAMIZOLE:Hepatic cirrhosis:0.5), (METHOXY POLYETHYLENE GLYCOL-EPOETIN BETA:Hepatic cirrhosis:0.2), (METHYLPHENIDATE HYDROCHLORIDE:Hepatic cirrhosis:0), (MINOCYCLINEMINOCYCLINE HYDROCHLORIDE:Hepatic cirrhosis:0.01), (MODAFINIL:Hepatic cirrhosis:0.08), (MOXIFLOXACIN HYDROCHLORIDE:Hepatic cirrhosis:0.09), (NABUMETONE:Hepatic cirrhosis:0.01), (NALOXONE HYDROCHLORIDEOXYCODONE HYDROCHLORIDE:Hepatic cirrhosis:0.62), (NIACIN:Hepatic cirrhosis:0.25), (NINTEDANIB:Hepatic cirrhosis:0.15), (OLODATEROL HYDROCHLORIDETIOTROPIUM BROMIDE MONOHYDRATE:Hepatic cirrhosis:0.24), (OMEGA-3-ACID ETHYL ESTERS:Hepatic cirrhosis:0.7), (OXCARBAZEPINE:Hepatic cirrhosis:0.92), (PANTOPRAZOLE MAGNESIUM:Hepatic cirrhosis:0.66), (PAROXETINEPAROXETINE HYDROCHLORIDE:Hepatic cirrhosis:0.78), (PATIROMER:Hepatic cirrhosis:0.03), (PAZOPANIB HYDROCHLORIDE:Hepatic cirrhosis:0.71), (PEGINTERFERON BETA-1A:Hepatic cirrhosis:0.23), (PENICILLIN:Hepatic cirrhosis:0.13), (PHENPROCOUMON:Hepatic cirrhosis:0.21), (PIROXICAM:Hepatic cirrhosis:0.04), (PONATINIB:Hepatic cirrhosis:0.28), (PRAMIPEXOLE:Hepatic cirrhosis:0.59), (RANOLAZINE:Hepatic cirrhosis:1.41), (RIOCIGUAT:Hepatic cirrhosis:0.94), (ROFLUMILAST:Hepatic cirrhosis:0.28), (SARPOGRELATE:Hepatic cirrhosis:0.37), (SODIUM OXYBATE:Hepatic cirrhosis:0.22), (SULFAMETHOXAZOLE:Hepatic cirrhosis:0.2), (TERBUTALINE SULFATE:Hepatic cirrhosis:0.46), (TERIFLUNOMIDE:Hepatic cirrhosis:0.45), (TETRACYCLINE:Hepatic cirrhosis:0.27), (THALIDOMIDE:Hepatic cirrhosis:0.27), (TIMOLOL:Hepatic cirrhosis:0.22), (TIZANIDINE HYDROCHLORIDE:Hepatic cirrhosis:0.85), (UMECLIDINIUM BROMIDEVILANTEROL TRIFENATATE:Hepatic cirrhosis:0.28), (VARENICLINE TARTRATE:Hepatic cirrhosis:0.69), (ZOLEDRONIC ACID:Hepatic cirrhosis:0.07), (CALCIUMVITAMIN D:Hepatic encephalopathy:0.07), (CANDESARTAN:Hepatic encephalopathy:0.15), (CANDESARTAN CILEXETIL:Hepatic encephalopathy:0.37), (CEPHALEXIN:Hepatic encephalopathy:0.06), (CERTOLIZUMAB PEGOL:Hepatic encephalopathy:0.95), (CLINDAMYCINCLINDAMYCIN PHOSPHATE:Hepatic encephalopathy:0.14), (DILTIAZEM:Hepatic encephalopathy:0.46), (DIVALPROEX SODIUM:Hepatic encephalopathy:0.75), (ENALAPRIL:Hepatic encephalopathy:1.2), (EPINEPHRINE:Hepatic encephalopathy:0.04), (ESZOPICLONE:Hepatic encephalopathy:0.39), (EVEROLIMUS:Hepatic encephalopathy:0.48), (EVOLOCUMAB:Hepatic encephalopathy:0.8), (EZETIMIBE:Hepatic encephalopathy:0.17), (IBRUTINIB:Hepatic encephalopathy:0.27), (ILOPROST:Hepatic encephalopathy:0.12), (ISOSORBIDE:Hepatic encephalopathy:0.1), (LERCANIDIPINE:Hepatic encephalopathy:0.24), (METHYLPREDNISOLONE SODIUM SUCCINATE:Hepatic encephalopathy:0.27), (NAPROXEN:Hepatic encephalopathy:1.33), (NEBIVOLOL:Hepatic encephalopathy:0.23), (PANTOPRAZOLE MAGNESIUM:Hepatic encephalopathy:0.13), (PHENOBARBITAL:Hepatic encephalopathy:0.15), (RALOXIFENE HYDROCHLORIDE:Hepatic encephalopathy:0.06), (RANOLAZINE:Hepatic encephalopathy:0.57), (RISEDRONATE SODIUM:Hepatic encephalopathy:0.09), (SODIUM OXYBATE:Hepatic encephalopathy:0.08), (SOTALOL:Hepatic encephalopathy:0.17), (TELMISARTAN:Hepatic encephalopathy:0.27), (TOCILIZUMAB:Hepatic encephalopathy:0.11), (VARENICLINE TARTRATE:Hepatic encephalopathy:0.11), (VERAPAMIL HYDROCHLORIDE:Hepatic encephalopathy:0.45), (ENALAPRIL:Hepatic failure:0.14), (NAPROXEN:Hepatic failure:0.01), (TERIFLUNOMIDE:Hepatic failure:0.27), (ACETAMINOPHENCODEINE PHOSPHATE:Hepatic function abnormal:0), (ADENINE:Hepatic function abnormal:0.25), (ALBUTEROL:Hepatic function abnormal:0.21), (AMBROXOLCLENBUTEROL:Hepatic function abnormal:0.12), (AMIODARONE:Hepatic function abnormal:0.2), (ANASTROZOLE:Hepatic function abnormal:0.7), (AZELNIDIPINE:Hepatic function abnormal:0.6), (BENDAMUSTINE:Hepatic function abnormal:0.14), (BEPRIDIL:Hepatic function abnormal:0.89), (BETAHISTINE MESILATE:Hepatic function abnormal:0.26), (BETAMIPRONPANIPENEM:Hepatic function abnormal:0.13), (BUDESONIDE:Hepatic function abnormal:0.53), (BUMETANIDE:Hepatic function abnormal:0.25), (BUPROPION HYDROCHLORIDE:Hepatic function abnormal:0.08), (CALCITRIOL:Hepatic function abnormal:0.28), (CARFILZOMIB:Hepatic function abnormal:0.06), (CEFOTIAM HYDROCHLORIDE:Hepatic function abnormal:0.01), (CEPHALEXIN:Hepatic function abnormal:0.48), (CLINDAMYCIN:Hepatic function abnormal:0.02), (CRIZOTINIB:Hepatic function abnormal:0.18), (DASATINIB:Hepatic function abnormal:0.26), (DILAZEP:Hepatic function abnormal:0.22), (DOBUTAMINE HYDROCHLORIDE:Hepatic function abnormal:0.08), (EPIRUBICIN:Hepatic function abnormal:0.12), (ETRETINATE:Hepatic function abnormal:0.76), (EVOLOCUMAB:Hepatic function abnormal:0.22), (EXEMESTANE:Hepatic function abnormal:0.02), (FLAVIN ADENINE DINUCLEOTIDE:Hepatic function abnormal:0.1), (FLUTICASONE PROPIONATE:Hepatic function abnormal:0.31), (FULVESTRANT:Hepatic function abnormal:0.36), (IBRITUMOMAB TIUXETAN:Hepatic function abnormal:0.01), (IBRUTINIB:Hepatic function abnormal:0.64), (IMIDAPRIL:Hepatic function abnormal:0.51), (INSULIN DETEMIR:Hepatic function abnormal:0.34), (IRINOTECAN:Hepatic function abnormal:0.21), (LEUCOVORIN CALCIUM:Hepatic function abnormal:0.38), (LYSOZYME HYDROCHLORIDE:Hepatic function abnormal:0.23), (MAXACALCITOL:Hepatic function abnormal:0.11), (METOPROLOL:Hepatic function abnormal:0.22), (METRONIDAZOLE:Hepatic function abnormal:0.64), (MORPHINE:Hepatic function abnormal:0.05), (NYSTATIN:Hepatic function abnormal:0.2), (OXCARBAZEPINE:Hepatic function abnormal:0.22), (PAZOPANIB HYDROCHLORIDE:Hepatic function abnormal:0.44), (POLYETHYLENE GLYCOLS:Hepatic function abnormal:0.19), (POTASSIUM:Hepatic function abnormal:0.35), (PRANLUKAST:Hepatic function abnormal:0.44), (PRAVASTATIN:Hepatic function abnormal:0.25), (RAMIPRIL:Hepatic function abnormal:0.84), (RAMUCIRUMAB:Hepatic function abnormal:0.71), (SCOPOLAMINE:Hepatic function abnormal:0), (TEMOCAPRIL HYDROCHLORIDE:Hepatic function abnormal:0.24), (TINZAPARIN SODIUM:Hepatic function abnormal:0.17), (TIPEPIDINE HIBENZATE:Hepatic function abnormal:0.13), (TOPIRAMATE:Hepatic function abnormal:0.29), (TOSUFLOXACIN TOSYLATE:Hepatic function abnormal:0.12), (TRAZODONE HYDROCHLORIDE:Hepatic function abnormal:0.27), (UROKINASE:Hepatic function abnormal:0.06), (WARFARIN SODIUM:Hepatic function abnormal:0.66), (CALCIUM CARBONATECHOLECALCIFEROL:Hepatic steatosis:0.26), (DEVICE:Hepatic steatosis:0.28), (IBRUTINIB:Hepatic steatosis:0.02), (IBUPROFEN:Hepatic steatosis:0.8), (INSULIN HUMAN:Hepatic steatosis:0.32), (IRBESARTAN:Hepatic steatosis:0.02), (LOVASTATIN:Hepatic steatosis:0.5), (POTASSIUM:Hepatic steatosis:0.1), (SECUKINUMAB:Hepatic steatosis:0.24), (TOCILIZUMAB:Hepatic steatosis:0.09), (WARFARIN:Hepatic steatosis:0.03), (ARIPIPRAZOLE:Hepatitis:0.15), (CERTOLIZUMAB PEGOL:Hepatitis:0.24), (DEXLANSOPRAZOLE:Hepatitis:0.13), (DICLOFENAC SODIUM:Hepatitis:0.02), (EVOLOCUMAB:Hepatitis:0.23), (FLUCONAZOLE:Hepatitis:0.02), (FLUTICASONE PROPIONATE:Hepatitis:0.06), (IRON:Hepatitis:0.08), (METRONIDAZOLE:Hepatitis:0.63), (PR AVASTATIN SODIUM:Hepatitis:0.46), (SACUBITRILVALSARTAN:Hepatitis:0.18), (TERIFLUNOMIDE: Hepatitis:0.58), (TOPIRAMATE:Hepatitis:0.56), (WARFARIN SODIUM:Hepatitis:0.25), (ABACAVIRLAMIVUDINE:Hepatitis B:0.45), (AMIKACIN SULFATE:Hepatitis B:0.04), (AMIODARONE:Hepatitis B:1.1), (AMLODIPINEPERINDOPRIL:Hepatitis B:0.01), (ANASTROZOLE:Hepatitis B:1.41), (APREPITANT:Hepatitis B:0.4), (BELATACEPT:Hepatitis B:0.21), (BETAMETHASONE DIPROPIONATE:Hepatitis B:0.3), (BISACODYL OR DOCUSATE SODIUM:Hepatitis B:0.35), (BUDESONIDEFORMOTEROL FUMARATE DIHYDRATE:Hepatitis B:0.22), (BUPRENORPHINE HYDROCHLORIDENALOXONE HYDROCHLORIDE:Hepatitis B:0.05), (CALCIUM ACETATE:Hepatitis B:0.48), (CALCIUM CITRATE:Hepatitis B:0.13), (CANDESARTAN CILEXETILHYDROCHLOROTHIAZIDE:Hepatitis B:1.25), (CEFOTAXIMECEFOTAXIME SODIUM:Hepatitis B:0.02), (CIMETIDINE:Hepatitis B:0.07), (COLCHICINE:Hepatitis B:0.71), (CORTISONEHYDROCORTISONE:Hepatitis B:0.36), (CYCLOBENZAPRINE HYDROCHLORIDE:Hepatitis B:0.06), (DABRAFENIB:Hepatitis B:0.06), (DACLIZUMAB:Hepatitis B:0.11), (DALTEPARIN:Hepatitis B:0.5), (DALTEPARIN SODIUM:Hepatitis B:0.35), (DIVALPROEX SODIUM:Hepatitis B:0.88), (DOCUSATE:Hepatitis B:0.1), (DOFETILIDE:Hepatitis B:0.04), (DOXAZOSINDOXAZOSIN MESYLATE:Hepatitis B:0.64), (DRONABINOL:Hepatitis B:0.04), (ELOTUZUMAB:Hepatitis B:0.36), (EMTRICITABINERILPIVIRINE HYDROCHLORIDETENOFOVIR DISOPROXIL FUMARATE:Hepatitis B:0.61), (EVOLOCUMAB:Hepatitis B:0.71), (EXEMESTANE:Hepatitis B:0.17), (FESOTERODINE FUMARATE:Hepatitis B:0.04), (FLECAINIDE:Hepatitis B:0.41), (FLUTICASONE:Hepatitis B:0.37), (FLUVASTATIN SODIUM:Hepatitis B:0.09), (GENTAMICIN:Hepatitis B:0.04), (GLYCOPYRRONIUMINDACATEROL:Hepatitis B:0.18), (HYDRALAZINE HYDROCHLORIDE:Hepatitis B:0.44), (HYDROCHLOROTHIAZIDELISINOPRIL:Hepatitis B:0.85), (HYDROXYZINE PAMOATE:Hepatitis B:0.05), (IBRITUMOMAB TIUXETAN:Hepatitis B:0.03), (ILOPROST:Hepatitis B:0.34), (IMATINIB MESYLATE:Hepatitis B:1.47), (INDAPAMIDE:Hepatitis B:0.41), (INFLUENZA VIRUS VACCINE:Hepatitis B:0.34), (IRINOTECAN HYDROCHLORIDE:Hepatitis B:0.26), (IRON:Hepatitis B:0.95), (ISOSORBIDE:Hepatitis B:0.46), (IXAZOMIB:Hepatitis B:0.14), (LABETALOLLABETALOL HYDROCHLORIDE:Hepatitis B:0.15), (LEVOLEUCOVORIN CALCIUM:Hepatitis B:0.05), (METOLAZONE:Hepatitis B:0.03), (MICAFUNGIN:Hepatitis B:0.1), (MOXIFLOXACIN HYDROCHLORIDE:Hepatitis B:0.81), (NADROPARIN CALCIUM:Hepatitis B:0.09), (OLODATEROL HYDROCHLORIDETIOTROPIUM BROMIDE MONOHYDRATE:Hepatitis B:0.03), (PANITUMUMAB:Hepatitis B:0.2), (PANTOPRAZOLE MAGNESIUM:Hepatitis B:0.35), (PAZOPANIB HYDROCHLORIDE:Hepatitis B:1.27), (PEGINTERFERON ALFA-2A OR PEGINTERFERON ALFA-2B:Hepatitis B:0.3), (PEMETREXED:Hepatitis B:0.26), (PHENOBARBITAL:Hepatitis B:0.42), (PITAVASTATIN CALCIUM:Hepatitis B:0.23), (PRIMIDONE:Hepatitis B:0.07), (PYRAZINAMIDE:Hepatitis B:0.75), (RANOLAZINE:Hepatitis B:0.65), (ROFLUMILAST:Hepatitis B:0.15), (SACUBITRILVALSARTAN:Hepatitis B:0.72), (SITAGLIPTIN:Hepatitis B:0.26), (TIGECYCLINE:Hepatitis B:0.14), (TIOTROPIUM:Hepatitis B:0.2), (TIZANIDINE HYDROCHLORIDE:Hepatitis B:0.07), (TOCILIZUMAB:Hepatitis B:0.3), (TOPIRAMATE:Hepatitis B:0.96), (TRAMETINIB:Hepatitis B:0.02), (VANCOMYCIN HYDROCHLORIDE:Hepatitis B:0.25), (ZONISAMIDE:Hepatitis B:0.13), (.ALPHA.1-PROTEINASE INHIBITOR HUMAN:Hepatitis C:0.49), (ABOBOTULINUMTOXINA:Hepatitis C:0.34), (ACAMPROSATE:Hepatitis C:0.03), (ACECLOFENAC:Hepatitis C:0.11), (ACETAMINOPHENMETHOCARBAMOL:Hepatitis C:0.16), (ACETAMINOPHENPROPOXYPHENE NAPSYLATE:Hepatitis C:0.78), (ADENOSINE:Hepatitis C:0.15), (ADO-TRASTUZUMAB EMTANSINE:Hepatitis C:0.78), (AGALSIDASE BETA:Hepatitis C:1.49), (ALBENDAZOLE:Hepatitis C:0.24), (ALISKIREN HEMIFUMARATEHYDROCHLOROTHIAZIDE:Hepatitis C:0.03), (AMBROXOLCLENBUTEROL:Hepatitis C:0.02), (AMIKACIN SULFATE:Hepatitis C:0.18), (AMINO ACIDSDEXTROSEELECTROLYTES NOS:Hepatitis C:1.02), (AMINOPHYLLINE:Hepatitis C:0.05), (AMLODIPINE BESYLATEHYDROCHLOROTHIAZIDEVALSARTAN:Hepatitis C:1.63), (AMLODIPINEVALSARTAN:Hepatitis C:1.35), (AMMONIUM CHLORIDEDEXTROMETHORPHANSODIUM CITRATE:Hepatitis C:0.28), (AMOXICILLINCLARITHROMYCINESOMEPRAZOLE:Hepatitis C:0.68), (AMPICILLIN:Hepatitis C:1.2), (ANAGRELIDE:Hepatitis C:0.07), (ANAGRELIDE HYDROCHLORIDE:Hepatitis C:0.41), (ARIPIPRAZOLE LAUROXIL:Hepatitis C:0.39), (ASCORBIC ACID/CHROMIUM/SELENIUM/VANADYL SULFATE:Hepatitis C:0), (ASPIRINMAGNESIUM OXIDE:Hepatitis C:0.84), (AXITINIB:Hepatitis C:1.24), (AZACITIDINE:Hepatitis C:0.3), (AZITHROMYCIN OR METHOTREXATE SODIUM:Hepatitis C:0.09), (BALSALAZIDE:Hepatitis C:0.02), (BALSALAZIDE DISODIUM:Hepatitis C:0.46), (BELATACEPT:Hepatitis C:0.73), (BENDAMUSTINE HYDROCHLORIDE:Hepatitis C:0.54), (BENSERAZIDELEVODOPA:Hepatitis C:0.02), (BEPRIDIL:Hepatitis C:0.35), (BERAPROST SODIUM:Hepatitis C:0.69), (BETHANECHOL CHLORIDE:Hepatitis C:0.6), (BIMATOPROSTTIMOLOL:Hepatitis C:0.12), (BLINATUMOMAB:Hepatitis C:0), (BOCOCIZUMAB:Hepatitis C:0.32), (BUPRENORPHINENALOXONE:Hepatitis C:0.27), (CALCIUM CARBONATEERGOCALCIFEROL:Hepatitis C:0.01), (CALCIUM CARBONATEERGOCALCIFEROLRETINOL:Hepatitis C:0.93), (CARBIDOPAENTACAPONELEVODOPA:Hepatitis C:0.14), (CARTEOLOL:Hepatitis C:0.46), (CEFOTAXIMECEFOTAXIME SODIUM:Hepatitis C:0.61), (CERITINIB:Hepatitis C:0.11), (CERIVASTATIN SODIUM:Hepatitis C:0.54), (CERTOPARIN SODIUM:Hepatitis C:0.07), (CHLORAMBUCIL:Hepatitis C:0.43), (CHLOROTHIAZIDE:Hepatitis C:0.4), (CHLORPHENIRAMINEHYDROCODONE:Hepatitis C:0.59), (CHOLESTEROL:Hepatitis C:0.06), (CICLOPIROX OLAMINE:Hepatitis C:0.1), (CIFENLINE:Hepatitis C:0.02), (CLIKSTAR:Hepatitis C:0.63), (CLOFAZIMINE:Hepatitis C:0.17), (CLOMIPRAMINE:Hepatitis C:0.1), (COAGULATION FACTOR VIIA RECOMBINANT HUMAN:Hepatitis C:0.02), (COLCHICINEDICYCLOMINE HYDROCHLORIDE:Hepatitis C:0.42), (COLISTIMETHATE SODIUM:Hepatitis C:0.26), (COLISTIN:Hepatitis C:0.03), (CRIZOTINIB:Hepatitis C:0.17), (DANTROLENE SODIUM:Hepatitis C:0.07), (DAPAGLIFLOZIN PROPANEDIOLMETFORMIN HYDROCHLORIDE:Hepatitis C:0.41), (DEFERIPRONE:Hepatitis C:0.05), (DEGARELIX ACETATE:Hepatitis C:0.02), (DEXAMETHASONE SODIUM PHOSPHATE:Hepatitis C:0.39), (DEXTROMETHORPHAN HYDROBROMIDEQUINIDINE SULFATE:Hepatitis C:0.29), (DIFLUNISAL:Hepatitis C:0.21), (DIPYRONEPROMETHAZINE HYDROCHLORIDE:Hepatitis C:0.22), (DOLASETRON MESYLATE:Hepatitis C:0.39), (DOXYLAMINE SUCCINATEPYRIDOXINE HYDROCHLORIDE:Hepatitis C:0.01), (DUTASTERIDETAMSULOSIN HYDROCHLORIDE:Hepatitis C:1.91), (ECONAZOLE:Hepatitis C:2.5), (EDARAVONE:Hepatitis C:0.14), (EDOXABAN TOSYLATE:Hepatitis C:2.48), (EFINACONAZOLE:Hepatitis C:0.46), (ELOTUZUMAB:Hepatitis C:0.93), (EMPAGLIFLOZINMETFORMIN HYDROCHLORIDE:Hepatitis C:0.18), (ESOMEPRAZOLE MAGNESIUMNAPROXEN:Hepatitis C:0.19), (ETHACRYNIC ACID:Hepatitis C:0.56), (ETHINYL ESTRADIOLNORETHINDRONE:Hepatitis C:0.7), (ETHINYL ESTRADIOLNORGESTREL:Hepatitis C:0.55), (ETHYL ICOSAPENTATEICOSAPENT:Hepatitis C:0.06), (ETRETINATE:Hepatitis C:0.94), (EUCALYPTOLMENTHOLMETHYL SALICYLATETHYMOL:Hepatitis C:0.18), (EVOLOCUMAB:Hepatitis C:2.59), (FENOTEROL HYDROBROMIDE:Hepatitis C:0.59), (FERRIC CITRATE ANHYDROUS:Hepatitis C:0.12), (FERRIC HYDROXIDE:Hepatitis C:0.47), (FERROUS BISGLYCINATE:Hepatitis C:0.37), (FLECAINIDE ACETATE:Hepatitis C:0.15), (FLUNARIZINE HYDROCHLORIDE:Hepatitis C:0.06), (FLUOROMETHOLONE:Hepatitis C:0.95), (FOLIC ACIDIRONMINERALSVITAMINS:Hepatitis C:0.09), (FONDAPARINUX:Hepatitis C:0.28), (FORMOTEROL FUMARATEMOMETASONE FUROATE:Hepatitis C:0.87), (FOSPHENYTOINFOSPHENYTOIN SODIUM:Hepatitis C:0.01), (FROVATRIPTAN SUCCINATE:Hepatitis C:0.04), (FURSULTIAMINE:Hepatitis C:0.05), (GLUTAMINE:Hepatitis C:0.18), (GUANFACINE:Hepatitis C:1.09), (HYDROCHLOROTHIAZIDERAMIPRIL:Hepatitis C:0.41), (HYDROCORTISONE ACETATE:Hepatitis C:1.23), (HYDROXOCOBALAMIN:Hepatitis C:0.08), (HYOSCYAMINE SULFATEMETHENAMINEMETHYLENE BLUEPHENYL SALICYLATESODIUM PHOSPHATE, MONOBASIC, MONOHYDRATE:Hepatitis C:0.14), (IDELALISIB:Hepatitis C:0.83), (IMIGLUCERASE:Hepatitis C:1.24), (IMIPRAMINE HYDROCHLORIDE:Hepatitis C:1.43), (INFLIXIMAB-DYYB:Hepatitis C:0.7), (INTERFERON GAMMA-1B:Hepatitis C:0.52), (IRONPOLYSACCHARIDES:Hepatitis C:0.37), (ISONIAZIDPYRAZINAMIDERIFAMPIN:Hepatitis C:0.36), (ISONIAZIDRIFAMPIN:Hepatitis C:0.87), (ITRACONAZOLESODIUM CHLORIDE:Hepatitis C:0.37), (IVABRADINE:Hepatitis C:0.89), (IVACAFTOR:Hepatitis C:0.07), (IVERMECTIN:Hepatitis C:0.86), (LACTASE:Hepatitis C:0.83), (LACTOBACILLUS ACIDOPHILUSLACTOBACILLUS DELBRUECKII SUBSP. BULGARICUS:Hepatitis C:0.36), (LANREOTIDE ACETATE:Hepatitis C:0.26), (LIDOCAINEPRILOCAINE:Hepatitis C:0.85), (LOMITAPIDE MESYLATE:Hepatitis C:1.27), (LUTETIUM LU-177:Hepatitis C:0.14), (MACITENTAN:Hepatitis C:0.03), (MAGNESIUM ASPARTATE HYDROCHLORIDE:Hepatitis C:0.55), (MELPERONE:Hepatitis C:0.45), (MEPOLIZUMAB:Hepatitis C:0.68), (MESNA:Hepatitis C:0.1), (METFORMIN HYDROCHLORIDESAXAGLIPTIN:Hepatitis C:0.05), (METHENAMINE:Hepatitis C:0.14), (METHENAMINE MANDELATE:Hepatitis C:0.18), (METHENOLONE ENANTHATE:Hepatitis C:0.53), (METRELEPTIN:Hepatitis C:0.95), (MEXILETINE:Hepatitis C:0.28), (MEXILETINE HYDROCHLORIDE:Hepatitis C:0.13), (MINOCYCLINE HYDROCHLORIDE:Hepatitis C:1.35), (MOEXIPRIL HYDROCHLORIDE:Hepatitis C:0.96), (MOLSIDOMINE:Hepatitis C:0.25), (NINTEDANIB:Hepatitis C:1.14), (NIRAPARIB:Hepatitis C:0.56), (NOREPINEPHRINE:Hepatitis C:0.38), (OXYBUTYNINOXYBUTYNIN CHLORIDE:Hepatitis C:0.7), (PALIPERIDONE PALMITATE:Hepatitis C:1.04), (PAMIDRONATE DISODIUM:Hepatitis C:1.57), (PARATHYROID HORMONE:Hepatitis C:0.12), (PATIROMER:Hepatitis C:0.96), (PEGINTERFERON BETA-1A:Hepatitis C:1.42), (PEMETREXED DISODIUM:Hepatitis C:1.29), (PENICILLIN V POTASSIUM:Hepatitis C:0.35), (PHENYTOIN SODIUM:Hepatitis C:1.57), (PILOCARPINE HYDROCHLORIDE:Hepatitis C:0.41), (PIMOBENDAN:Hepatitis C:0.61), (PINDOLOL:Hepatitis C:0.05), (POTASSIUM CITRATETRISODIUM CITRATE DIHYDRATE:Hepatitis C:0.03), (POVIDONE:Hepatitis C:0.16), (PRAMLINTIDE ACETATE:Hepatitis C:0.02), (PREDNISOLONE SODIUM PHOSPHATE:Hepatitis C:0.8), (PREDNISOLONE SODIUM SUCCINATE:Hepatitis C:0.18), (PRISTINAMYCIN:Hepatitis C:1.16), (PROPATYL NITRATE:Hepatitis C:0.05), (PROTRIPTYLINE HYDROCHLORIDE:Hepatitis C:0.68), (PYRIDOSTIGMINE:Hepatitis C:0.01), (QUINACRINE:Hepatitis C:0.75), (REGORAFENIB:Hepatitis C:0.25), (RISEDRONIC ACID:Hepatitis C:0.07), (RIVASTIGMINE TARTRATE:Hepatitis C:1.66), (ROFECOXIB:Hepatitis C:0.45), (ROFLUMILAST:Hepatitis C:1.76), (SALMETEROL:Hepatitis C:0.98), (SARPOGRELATE:Hepatitis C:1.11), (SELEGILINE:Hepatitis C:0.7), (SELEGILINE HYDROCHLORIDE:Hepatitis C:0.34), (SELEXIPAG:Hepatitis C:0.33), (SIBUTRAMINE HYDROCHLORIDE:Hepatitis C:0.17), (SIPULEUCEL-T:Hepatitis C:0.14), (SODIUM BICARBONATESODIUM CHLORIDE:Hepatitis C:0.06), (SODIUM CITRATE:Hepatitis C:0.02), (SORBITOL:Hepatitis C:0.25), (STREPTOMYCINSTREPTOMYCIN SULFATE:Hepatitis C:0.07), (SULFACETAMIDE SODIUMSULFUR:Hepatitis C:0.06), (TAFAMIDIS MEGLUMINE:Hepatitis C:0.07), (TASIMELTEON:Hepatitis C:0.36), (TEDUGLUTIDEWATER:Hepatitis C:0.53), (TELOTRISTAT ETHYL:Hepatitis C:0.15), (TETRAZEPAM:Hepatitis C:0.12), (THALIDOMIDE:Hepatitis C:1.83), (THEOPHYLLINE:Hepatitis C:0.05), (THIOCTIC ACID:Hepatitis C:0.73), (TIBOLONE:Hepatitis C:0.32), (TIMOLOLTRAVOPROST:Hepatitis C:0.34), (TOFACITINIB:Hepatitis C:0.09), (TOLTERODINE:Hepatitis C:0.77), (TROSPIUM:Hepatitis C:0.15), (UMECLIDINIUM BROMIDE:Hepatitis C:1.02), (VEMURAFENIB:Hepatitis C:0.54), (YEAST:Hepatitis C:0.68), (ZALCITABINE:Hepatitis C:0.28), (ZALEPLON:Hepatitis C:0.14), (ZILEUTON:Hepatitis C:0.52), (ZIPRASIDONE HYDROCHLORIDE:Hepatitis C:0.04), (ACETAZOLAMIDE:Hepatocellular carcinoma:0.08), (ADO-TRASTUZUMAB EMTANSINE:Hepatocellular carcinoma:0.64), (AFATINIB:Hepatocellular carcinoma:0.38), (ALENDRONIC ACID:Hepatocellular carcinoma:0.23), (ALISKIREN:Hepatocellular carcinoma:0.5), (ALUMINUM HYDROXIDEDIMETHICONEMAGNESIUM HYDROXIDE:Hepatocellular carcinoma:0.13), (AMISULPRIDE:Hepatocellular carcinoma:0.01), (AMPICILLIN:Hepatocellular carcinoma:0.18), (ANASTROZOLE:Hepatocellular carcinoma:1.36), (APREPITANT:Hepatocellular carcinoma:0.54), (ASUNAPREVIR:Hepatocellular carcinoma:0.57), (BENAZEPRILBENAZEPRIL HYDROCHLORIDE:Hepatocellular carcinoma:0.16), (BOCEPREVIR:Hepatocellular carcinoma:0.02), (BORTEZOMIB:Hepatocellular carcinoma:0.36), (BUDESONIDEFORMOTEROL FUMARATE:Hepatocellular carcinoma:0.04), (CALCIUM ACETATE:Hepatocellular carcinoma:0.36), (CALCIUM CARBONATECHOLECALCIFEROLMAGNESIUM CARBONATE:Hepatocellular carcinoma:0.1), (CALCIUM CITRATE:Hepatocellular carcinoma:0.15), (CANAGLIFLOZINMETFORMIN HYDROCHLORIDE:Hepatocellular carcinoma:0.21), (CARVEDILOL PHOSPHATE:Hepatocellular carcinoma:0.19), (CERITINIB:Hepatocellular carcinoma:0.39), (CERTOLIZUMAB PEGOL:Hepatocellular carcinoma:1.08), (CHLORTHALIDONE:Hepatocellular carcinoma:0.08), (CLOZAPINE:Hepatocellular carcinoma:0.7), (CORTISONEHYDROCORTISONE:Hepatocellular carcinoma:0.75), (CYCLOBENZAPRINE HYDROCHLORIDE:Hepatocellular carcinoma:0.47), (DABRAFENIB:Hepatocellular carcinoma:0.56), (DAPAGLIFLOZIN PROPANEDIOL:Hepatocellular carcinoma:0.04), (DASATINIB:Hepatocellular carcinoma:1.38), (DEVICE:Hepatocellular carcinoma:0.1), (DEXAMETHASONE ACETATE:Hepatocellular carcinoma:0.11), (DIGITOXIN:Hepatocellular carcinoma:0.27), (DOCUSATE CALCIUM:Hepatocellular carcinoma:0.22), (DOFETILIDE:Hepatocellular carcinoma:0.31), (DRONEDARONE:Hepatocellular carcinoma:0.11), (ECONAZOLE:Hepatocellular carcinoma:0.26), (EDOXABAN TOSYLATE:Hepatocellular carcinoma:0.95), (ELETRIPTAN HYDROBROMIDE:Hepatocellular carcinoma:0.02), (EPINEPHRINE:Hepatocellular carcinoma:0.51), (EVOLOCUMAB:Hepatocellular carcinoma:0.65), (EXEMESTANE:Hepatocellular carcinoma:0.27), (FESOTERODINE FUMARATE:Hepatocellular carcinoma:0.27), (FLECAINIDE:Hepatocellular carcinoma:1.02), (FLUDROCORTISONE ACETATE:Hepatocellular carcinoma:0.19), (FLUOXETINEFLUOXETINE HYDROCHLORIDE:Hepatocellular carcinoma:0.43), (FLUVASTATIN SODIUM:Hepatocellular carcinoma:0.19), (FUSIDIC ACID:Hepatocellular carcinoma:0.64), (GEFITINIB:Hepatocellular carcinoma:0.24), (GLYCOPYRRONIUMINDACATEROL:Hepatocellular carcinoma:0.43), (GUAIFENESIN:Hepatocellular carcinoma:0.14), (HEPARIN CALCIUM:Hepatocellular carcinoma:0.64), (HERBALSSAW PALMETTO:Hepatocellular carcinoma:0.09), (HYDROXYCHLOROQUINE DIPHOSPHATE:Hepatocellular carcinoma:0.13), (ILOPROST:Hepatocellular carcinoma:0.19), (IMATINIB MESYLATE:Hepatocellular carcinoma:1.46), (INTERFERON:Hepatocellular carcinoma:0.54), (INTERFERON ALFA-2A:Hepatocellular carcinoma:0.31), (IRINOTECAN HYDROCHLORIDE:Hepatocellular carcinoma:1), (ISOSORBIDE:Hepatocellular carcinoma:0.71), (LAPATINIB DITOSYLATE:Hepatocellular carcinoma:0.16), (LEVALBUTEROL HYDROCHLORIDE:Hepatocellular carcinoma:0.2), (MAGNESIUM SULFATE:Hepatocellular carcinoma:0.1), (METAXALONE:Hepatocellular carcinoma:0.11), (METFORMIN HYDROCHLORIDEVILDAGLIPTIN:Hepatocellular carcinoma:0.1), (MORPHINE SULFATENALTREXONE HYDROCHLORIDE:Hepatocellular carcinoma:0.06), (MOXIFLOXACIN HYDROCHLORIDE:Hepatocellular carcinoma:0.9), (NILOTINIB:Hepatocellular carcinoma:1.69), (NIRAPARIB:Hepatocellular carcinoma:0.35), (OMEGA-3-ACID ETHYL ESTERS:Hepatocellular carcinoma:0.72), (PANITUMUMAB:Hepatocellular carcinoma:0.54), (PANTOPRAZOLE MAGNESIUM:Hepatocellular carcinoma:0.78), (PAROXETINE:Hepatocellular carcinoma:0.61), (PAROXETINEPAROXETINE HYDROCHLORIDE:Hepatocellular carcinoma:0.34), (PEMETREXED:Hepatocellular carcinoma:0.6), (PEMETREXED DISODIUM:Hepatocellular carcinoma:0.35), (PENTOXIFYLLINE:Hepatocellular carcinoma:0.09), (PLANTAGO SEED:Hepatocellular carcinoma:0.08), (PRAMIPEXOLE:Hepatocellular carcinoma:0.26), (PRAMIPEXOLE DIHYDROCHLORIDE:Hepatocellular carcinoma:0.15), (PRIMIDONE:Hepatocellular carcinoma:0.5), (PRISTINAMYCIN:Hepatocellular carcinoma:0.13), (PYRAZINAMIDE:Hepatocellular carcinoma:0.67), (RALOXIFENE HYDROCHLORIDE:Hepatocellular carcinoma:0.06), (RANOLAZINE:Hepatocellular carcinoma:0.84), (ROFLUMILAST:Hepatocellular carcinoma:0.34), (SECUKINUMAB:Hepatocellular carcinoma:0.56), (SEVELAMER CARBONATE:Hepatocellular carcinoma:0.28), (SEVELAMER HYDROCHLORIDE:Hepatocellular carcinoma:0.23), (SODIUM OXYBATE:Hepatocellular carcinoma:0.16), (SULFAMETHOXAZOLE:Hepatocellular carcinoma:0.28), (TELOTRISTAT ETHYL:Hepatocellular carcinoma:0.22), (TINZAPARIN:Hepatocellular carcinoma:0.27), (TIPIRACIL HYDROCHLORIDETRIFLURIDINE:Hepatocellular carcinoma:0.27), (TOFACITINIB CITRATE:Hepatocellular carcinoma:0.05), (TOPIRAMATE:Hepatocellular carcinoma:0.73), (TRIAMCINOLONE:Hepatocellular carcinoma:0.11), (URAPIDIL:Hepatocellular carcinoma:0.27), (VEMURAFENIB:Hepatocellular carcinoma:0.67), (VINORELBINE TARTRATE:Hepatocellular carcinoma:0.08), (ACETAZOLAMIDE:HER-2 positive breast cancer:0.21), (ALCOHOL:HER-2 positive breast cancer:0.37), (AMISULPRIDE:HER-2 positive breast cancer:0.21), (BICALUTAMIDE:HER-2 positive breast cancer:0.38), (CALCITRIOL:HER-2 positive breast cancer:0.46), (CANAKINUMAB:HER-2 positive breast cancer:0.08), (CARFILZOMIB:HER-2 positive breast cancer:0.32), (CARVEDILOL PHOSPHATE:HER-2 positive breast cancer:0.1), (CEFOTAXIME SODIUM:HER-2 positive breast cancer:0.33), (CERTOLIZUMAB PEGOL:HER-2 positive breast cancer:1.45), (CETUXIMAB:HER-2 positive breast cancer:0), (CHOLESTYRAMINE:HER-2 positive breast cancer:0.91), (CILASTATIN SODIUMIMIPENEM:HER-2 positive breast cancer:0.37), (COLCHICINE:HER-2 positive breast cancer:0.76), (CRIZOTINIB:HER-2 positive breast cancer:0.26), (DABRAFENIB:HER-2 positive breast cancer:0.05), (DOXAZOSIN MESYLATE:HER-2 positive breast cancer:0.17), (ELETRIPTAN HYDROBROMIDE:HER-2 positive breast cancer:0.16), (ESTROGENS, CONJUGATED:HER-2 positive breast cancer:0.09), (EVOLOCUMAB:HER-2 positive breast cancer:0.01), (FESOTERODINE FUMARATE:HER-2 positive breast cancer:0.14), (FLUOCINONIDE:HER-2 positive breast cancer:0.02), (FLUTICASONE:HER-2 positive breast cancer:0.3), (FLUTICASONE FUROATE:HER-2 positive breast cancer:0.09), (GLYBURIDE:HER-2 positive breast cancer:0.27), (GLYCOPYRRONIUMINDACATEROL:HER-2 positive breast cancer:0.23), (HYDROCODONE:HER-2 positive breast cancer:0.7), (HYDROXYCHLOROQUINE DIPHOSPHATE:HER-2 positive breast cancer:0.11), (IBRUTINIB:HER-2 positive breast cancer:1.4), (ILOPROST:HER-2 positive breast cancer:0.04), (IMATINIB MESYLATE:HER-2 positive breast cancer:0.14), (IRINOTECAN HYDROCHLORIDE:HER-2 positive breast cancer:0.42), (ISOSORBIDE:HER-2 positive breast cancer:0.08), (LEDIPASVIRSOFOSBUVIR:HER-2 positive breast cancer:0.11), (LENALIDOMIDE:HER-2 positive breast cancer:0.35), (LEVALBUTEROL HYDROCHLORIDE:HER-2 positive breast cancer:0.11), (LEVOLEUCOVORIN CALCIUM:HER-2 positive breast cancer:0.02), (LITHIUM CARBONATE:HER-2 positive breast cancer:0.67), (LOVASTATIN:HER-2 positive breast cancer:0.89), (METAXALONE:HER-2 positive breast cancer:0.1), (METHADONE HYDROCHLORIDE:HER-2 positive breast cancer:0.58), (METHOCARBAMOL:HER-2 positive breast cancer:0.19), (METHYLPREDNISOLONE ACETATE:HER-2 positive breast cancer:0.82), (NABUMETONE:HER-2 positive breast cancer:0.06), (NIACIN:HER-2 positive breast cancer:0.07), (NILOTINIB:HER-2 positive breast cancer:1.41), (OLAPARIB:HER-2 positive breast cancer:0.35), (OMEGA-3-ACID ETHYL ESTERS:HER-2 positive breast cancer:0.05), (OXYBUTYNIN CHLORIDE:HER-2 positive breast cancer:0.11), (PAZOPANIB HYDROCHLORIDE:HER-2 positive breast cancer:0.71), (PEMETREXED:HER-2 positive breast cancer:0.23), (PEMETREXED DISODIUM:HER-2 positive breast cancer:0.23), (PENICILLIN:HER-2 positive breast cancer:0.83), (PHENOBARBITAL:HER-2 positive breast cancer:0.17), (PRAMIPEXOLE:HER-2 positive breast cancer:0.21), (PREDNISOLONEPREDNISOLONE ACETATEPREDNISOLONE SODIUM SUCCINATE:HER-2 positive breast cancer:0.25), (PROGESTERONE:HER-2 positive breast cancer:0.38), (RAMUCIRUMAB:HER-2 positive breast cancer:0.05), (RANOLAZINE:HER-2 positive breast cancer:0.03), (REGORAFENIB:HER-2 positive breast cancer:0.08), (ROFLUMILAST:HER-2 positive breast cancer:0.33), (SACUBITRILVALSARTAN:HER-2 positive breast cancer:0.11), (SECUKINUMAB:HER-2 positive breast cancer:0.14), (SODIUM OXYBATE:HER-2 positive breast cancer:0.03), (SOTALOL:HER-2 positive breast cancer:0.78), (SULFAMETHOXAZOLE:HER-2 positive breast cancer:0.17), (TAMSULOSIN HYDROCHLORIDE:HER-2 positive breast cancer:0.29), (THALIDOMIDE:HER-2 positive breast cancer:0.05), (TIZANIDINE:HER-2 positive breast cancer:0.2), (TIZANIDINE HYDROCHLORIDE:HER-2 positive breast cancer:0.59), (TRAMETINIB:HER-2 positive breast cancer:0.01), (VARENICLINE TARTRATE:HER-2 positive breast cancer:0.27), (BENAZEPRIL HYDROCHLORIDE:Hereditary angioedema:0.12), (BENZONATATE:Hereditary angioedema:0.09), (BISACODYL OR DOCUSATE SODIUM:Hereditary angioedema:0.58), (BISOPROLOL:Hereditary angioedema:0.03), (BUDESONIDEFORMOTEROL FUMARATE:Hereditary angioedema:0.04), (BUPRENORPHINE HYDROCHLORIDENALOXONE HYDROCHLORIDE:Hereditary angioedema:0.02), (CALCIUM ACETATE:Hereditary angioedema:0.11), (CALCIUM CARBONATEVITAMIN D:Hereditary angioedema:0.01), (CANAKINUMAB:Hereditary angioedema:0.23), (CANDESARTAN CILEXETIL:Hereditary angioedema:0.25), (CARVEDILOL PHOSPHATE:Hereditary angioedema:0.42), (CIPROFLOXACIN:Hereditary angioedema:0.01), (COLCHICINE:Hereditary angioedema:0.44), (DACLIZUMAB:Hereditary angioedema:0.03), (DOXAZOSIN MESYLATE:Hereditary angioedema:0.11), (ENALAPRIL:Hereditary angioedema:1.37), (ENOXAPARIN:Hereditary angioedema:0.56), (ESZOPICLONE:Hereditary angioedema:1.49), (EVOLOCUMAB:Hereditary angioedema:0.71), (EZETIMIBESIMVASTATIN:Hereditary angioedema:0.32), (FISH OIL\OMEGA-3 FATTY ACIDS:Hereditary angioedema:0.04), (FLUOCINONIDE:Hereditary angioedema:0.04), (FLUTICASONE FUROATESALMETEROL XINAFOATE:Hereditary angioedema:0.1), (FORMOTEROL FUMARATE:Hereditary angioedema:0.07), (GLUCOSAMINE:Hereditary angioedema:0), (HYDROCHLOROTHIAZIDELISINOPRIL:Hereditary angioedema:0.46), (HYDROCHLOROTHIAZIDELOSARTAN POTASSIUM:Hereditary angioedema:0.13), (IBRUTINIB:Hereditary angioedema:0.16), (ILOPROST:Hereditary angioedema:0.24), (IRBESARTAN:Hereditary angioedema:0.14), (ISOSORBIDE:Hereditary angioedema:0.38), (LIOTHYRONINE SODIUM:Hereditary angioedema:0.49), (LOVASTATIN:Hereditary angioedema:1.32), (MEPOLIZUMAB:Hereditary angioedema:0.29), (METHENAMINE HIPPURATE:Hereditary angioedema:0.08), (MOXIFLOXACIN HYDROCHLORIDE:Hereditary angioedema:0.3), (NILOTINIB:Hereditary angioedema:0.23), (PAROXETINE:Hereditary angioedema:0.44), (PEGFILGRASTIM:Hereditary angioedema:0.11), (PENICILLIN:Hereditary angioedema:0.78), (PHENOBARBITAL:Hereditary angioedema:1.22), (PONATINIB:Hereditary angioedema:0.24), (PRAMIPEXOLE:Hereditary angioedema:0.46), (PYRIDOXINE HYDROCHLORIDE:Hereditary angioedema:0.33), (SECUKINUMAB:Hereditary angioedema:0.07), (SOTALOL:Hereditary angioedema:0.13), (TELMISARTAN:Hereditary angioedema:0.3), (TERIFLUNOMIDE:Hereditary angioedema:0.08), (TETRACYCLINE:Hereditary angioedema:0.08), (TORSEMIDE:Hereditary angioedema:0.6), (UMECLIDINIUM BROMIDEVILANTEROL TRIFENATATE:Hereditary angioedema:0.6), (ZINC:Hereditary angioedema:0.15), (BISOPROLOL FUMARATE:Hernia:0.06), (BUPROPION HYDROCHLORIDE:Hernia:0.52), (DALFAMPRIDINE:Hernia:0.72), (EVOLOCUMAB:Hernia: 0.44), (FERROUS SULFATEFERROUS SULFATE, DRIED:Hernia:0.24), (FISH OIL:Hernia:0.05), (IBRUTINIB:Hernia:0.44), (METHYLPREDNISOLONE SODIUM SUCCINATE:Hernia:0.17), (TAMSULOSIN:Hernia:0.2), (WARFARIN:Hernia:0.8), (ABACAVI R SULFATEDOLUTEGRAVIR SODIUMLAMIVUDINE:Herpes simplex:0.09), (ACETAMINOPHENOXYCODONEOXYCODONE HYDROCHLORIDE:Herpes simplex:0.11), (ACETAZOLAMIDE:Herpes simplex:0.1), (ALENDRONIC ACID:Herpes simplex:0.12), (ANASTROZOLE:Herpes simplex:0.98), (ATROPINE SULFATEDIPHENOXYLATE HYDROCHLORIDE:Herpes simplex:0.22), (BENAZEPRILBENAZEPRIL HYDROCHLORIDE:Herpes simplex:0.02), (BENDAMUSTINE HYDROCHLORIDE:Herpes simplex:0.31), (BEVACIZUMAB:Herpes simplex:0.12), (BISOPROLOL:Herpes simplex:0.41), (BISOPROLOL FUMARATE:Herpes simplex:0.5), (BUPRENORPHINE HYDROCHLORIDE:Herpes simplex:0.55), (CALCITRIOL:Herpes simplex:0.65), (CALCIUM ACETATE:Herpes simplex:0.54), (CANAKINUMAB:Herpes simplex:0.31), (CANDESARTAN:Herpes simplex:0.88), (CANDESARTAN CILEXETILHYDROCHLOROTHIAZIDE:Herpes simplex:0.54), (CARVEDILOL PHOSPHATE:Herpes simplex:0.18), (CEFOTAXIME SODIUM:Herpes simplex:0.24), (CIMETIDINE:Herpes simplex:0.31), (CLOZAPINE:Herpes simplex:0.43), (DALFAMPRIDINE:Herpes simplex:0.66), (DASATINIB:Herpes simplex:1.03), (DESVENLAFAXINE SUCCINATE:Herpes simplex:0.1), (DEVICE:Herpes simplex:0.07), (DOLUTEGRAVIR:Herpes simplex:0.1), (DOXAZOSINDOXAZOSIN MESYLATE:Herpes simplex:0.22), (DULOXETINE:Herpes simplex:0.75), (ECONAZOLE:Herpes simplex:0.04), (EDOXABAN TOSYLATE:Herpes simplex:0), (ERYTHROPOIETIN:Herpes simplex:0.79), (ESTROGENS, CONJUGATEDMEDROXYPROGESTERONE ACETATE:Herpes simplex:0.17), (ETORICOXIB:Herpes simplex:0.26), (EZETIMIBESIMVASTATIN:Herpes simplex:0.21), (FERROUS SULFATE:Herpes simplex:1.2), (FESOTERODINE FUMARATE:Herpes simplex:0.21), (FLECAINIDE:Herpes simplex:0.16), (FLUOXETINEFLUOXETINE HYDROCHLORIDE:Herpes simplex:0.32), (FLUTICASONE FUROATESALMETEROL XINAFOATE:Herpes simplex:0.01), (GLYBURIDE:Herpes simplex:0.22), (HYDROCHLOROTHIAZIDELISINOPRIL:Herpes simplex:1.08), (HYDROCHLOROTHIAZIDEVALSARTAN:Herpes simplex:0.24), (HYDROXYCHLOROQUINE DIPHOSPHATE:Herpes simplex:0.17), (HYDROXYZINE PAMOATE:Herpes simplex:0.83), (IBRITUMOMAB TIUXETAN:Herpes simplex:0.19), (IMIPENEM:Herpes simplex:0.28), (IMIPRAMINE HYDROCHLORIDE:Herpes simplex:0.38), (INDOMETHACIN:Herpes simplex:0.65), (ISOSORBIDE:Herpes simplex:0.39), (ITRACONAZOLE:Herpes simplex:0.12), (LERCANIDIPINE:Herpes simplex:0.79), (LERCANIDIPINE HYDROCHLORIDE:Herpes simplex:0.19), (LINEZOLID:Herpes simplex:0.21), (METHENAMINE HIPPURATE:Herpes simplex:0.29), (METHIMAZOLE:Herpes simplex:0.03), (NADOLOL:Herpes simplex:0.21), (NEBIVOLOL:Herpes simplex:0.42), (NEBIVOLOL HYDROCHLORIDE:Herpes simplex:0.44), (NEVIRAPINE:Herpes simplex:0.25), (OSELTAMIVIR:Herpes simplex:0.16), (OXCARBAZEPINE:Herpes simplex:1.76), (OXYBUTYNIN CHLORIDE:Herpes simplex:0.43), (PANTOPRAZOLE MAGNESIUM:Herpes simplex:0.58), (PEGINTERFERON BETA-1A:Herpes simplex:0.08), (PENICILLIN:Herpes simplex:0.4), (PITAVASTATIN CALCIUM:Herpes simplex:0.04), (PRAMIPEXOLE DIHYDROCHLORIDE:Herpes simplex:0.27), (PREDNISOLONEPREDNISOLONE ACETATEPREDNISOLONE SODIUM SUCCINATE:Herpes simplex:0.05), (PRIMIDONE:Herpes simplex:0.74), (QUETIAPINE:Herpes simplex:0.53), (RALOXIFENE HYDROCHLORIDE:Herpes simplex:0.14), (RANOLAZINE:Herpes simplex:0.66), (RIFAMPIN:Herpes simplex:0.78), (RISEDRONATE SODIUM:Herpes simplex:0.44), (ROFLUMILAST:Herpes simplex:0.14), (SOTALOL:Herpes simplex:0.57), (SUCRALFATE:Herpes simplex:0.39), (TERBUTALINE SULFATE:Herpes simplex:0.25), (TERIFLUNOMIDE:Herpes simplex:0.71), (TETRACYCLINE:Herpes simplex:0.45), (TIMOLOL:Herpes simplex:0.19), (TOCILIZUMAB:Herpes simplex:0.36), (TOLTERODINE TARTRATE:Herpes simplex:0.32), (UMECLIDINIUM BROMIDEVILANTEROL TRIFENATATE:Herpes simplex:0.42), (VARENICLINE TARTRATE:Herpes simplex:1.22), (VITAMIN A:Herpes simplex:0.04), (ACETAZOLAMIDE:Herpes virus infection:0.27), (ALCOHOL:Herpes virus infection:0.23), (ALENDRONIC ACID:Herpes virus infection:0.07), (AZITHROMYCIN DIHYDRATE:Herpes virus infection:0.4), (BENZONATATE:Herpes virus infection:0.17), (BISOPROLOL FUMARATE:Herpes virus infection:1.02), (BLINATUMOMAB:Herpes virus infection:0.29), (BROMAZEPAM:Herpes virus infection:0.18), (BUMETANIDE:Herpes virus infection:0.62), (CALCIUM ACETATE:Herpes virus infection:0.21), (CALCIUM CARBONATEVITAMIN D:Herpes virus infection:0.42), (CANAGLIFLOZINMETFORMIN HYDROCHLORIDE:Herpes virus infection:0.26), (CANDESARTAN CILEXETIL:Herpes virus infection:0.97), (CARVEDILOL PHOSPHATE:Herpes virus infection:0.04), (CEFEPIME HYDROCHLORIDE:Herpes virus infection:0.03), (CEFOTAXIME SODIUM:Herpes virus infection:0.24), (CIMETIDINE:Herpes virus infection:0.37), (CLINDAMYCIN:Herpes virus infection:0.56), (CLOZAPINE:Herpes virus infection:0.44), (CORTISONEHYDROCORTISONE:Herpes virus infection:0.57), (DAPAGLIFLOZIN PROPANEDIOL:Herpes virus infection:0.35), (DASATINIB:Herpes virus infection:0.89), (DEVICE:Herpes virus infection:0.6), (DIETARY SUPPLEMENT:Herpes virus infection:0.09), (DIMENHYDRINATE:Herpes virus infection:0.85), (DOCUSATE:Herpes virus infection:0.54), (DOLUTEGRAVIR:Herpes virus infection:0.51), (DOXAZOSIN MESYLATE:Herpes virus infection:0.42), (DOXEPIN HYDROCHLORIDE:Herpes virus infection:0.07), (DULAGLUTIDE:Herpes virus infection:0.13), (DUTASTERIDE:Herpes virus infection:0.73), (ECONAZOLE:Herpes virus infection:0.08), (EMPAGLIFLOZINLINAGLIPTIN:Herpes virus infection:0.26), (EVEROLIMUS:Herpes virus infection:0.35), (EVOLOCUMAB:Herpes virus infection:1.11), (FERROUS SULFATE:Herpes virus infection:1.3), (FESOTERODINE FUMARATE:Herpes virus infection:0.37), (FLUTICASONE FUROATESALMETEROL XINAFOATE:Herpes virus infection:0.21), (FLUTICASONE FUROATEVILANTEROL TRIFENATATE:Herpes virus infection:0.25), (HALOPERIDOL:Herpes virus infection:0.26), (HERBALSTURMERIC:Herpes virus infection:0.03), (HYDRALAZINE HYDROCHLORIDE:Herpes virus infection:0.57), (HYDROCHLOROTHIAZIDETELMISARTAN:Herpes virus infection:0.09), (HYDROXYCHLOROQUINE DIPHOSPHATE:Herpes virus infection:0.11), (HYDROXYZINE PAMOATE:Herpes virus infection:1.02), (IMATINIB MESYLATE:Herpes virus infection:0.5), (IMIPENEM:Herpes virus infection:0.01), (IMIPRAMINE HYDROCHLORIDE:Herpes virus infection:0.4), (INDAPAMIDEPERINDOPRIL:Herpes virus infection:0.35), (IRBESARTAN:Herpes virus infection:0.71), (LABETALOL HYDROCHLORIDE:Herpes virus infection:0.31), (LACOSAMIDE:Herpes virus infection:0.17), (LERCANIDIPINE HYDROCHLORIDE:Herpes virus infection:0.62), (LEVALBUTEROL HYDROCHLORIDE:Herpes virus infection:0.49), (MEDROXYPROGESTERONE ACETATE:Herpes virus infection:0.51), (METHENAMINE HIPPURATE:Herpes virus infection:0.29), (MOXIFLOXACIN HYDROCHLORIDE:Herpes virus infection:0.87), (NALOXONE HYDROCHLORIDEOXYCODONE HYDROCHLORIDE:Herpes virus infection:0.42), (NEBIVOLOL:Herpes virus infection:0.6), (NILOTINIB:Herpes virus infection:1), (NORTRIPTYLINE HYDROCHLORIDE:Herpes virus infection:0.02), (PAROXETINEPAROXETINE HYDROCHLORIDE:Herpes virus infection:0.8), (PAZOPANIB HYDROCHLORIDE:Herpes virus infection:0.1), (PIROXICAM:Herpes virus infection:0.04), (PONATINIB:Herpes virus infection:0.57), (PRAZOSIN:Herpes virus infection:0.09), (PRIMIDONE:Herpes virus infection:1.13), (PYRIDOXINE:Herpes virus infection:0.3), (RANOLAZINE:Herpes virus infection:0.9), (ROFLUMILAST:Herpes virus infection:0.39), (SACUBITRILVALSARTAN:Herpes virus infection:0.64), (SOLIFENACIN SUCCINATE:Herpes virus infection:0.19), (SOTALOL:Herpes virus infection:0.67), (TERBUTALINE SULFATE:Herpes virus infection:0.43), (TERIFLUNOMIDE:Herpes virus infection:0.78), (THALIDOMIDE:Herpes virus infection:0.67), (TIOTROPIUM:Herpes virus infection:0.03), (TOBRAMYCIN:Herpes virus infection:0.07), (UMECLIDINIUM BROMIDEVILANTEROL TRIFENATATE:Herpes virus infection:0.14), (VILAZODONE HYDROCHLORIDE:Herpes virus infection:0.39), (VITAMIN A:Herpes virus infection:0.39), (ZINC:Herpes virus infection:0.35), (ACENOCOUMAROL:Herpes zoster:1.24), (ADENINE:Herpes zoster:0.53), (AGALSIDASE BETA:Herpes zoster:1.07), (ALBIGLUTIDE:Herpes zoster:0.17), (ALBUMIN BOVINETANNIC ACID:Herpes zoster:0.44), (ALCOHOL:Herpes zoster:0.27), (ALISKIREN HEMIFUMARATE:Herpes zoster:0.93), (ALUMINUM SILICATE:Herpes zoster:0.38), (AMILORIDE HYDROCHLORIDEHYDROCHLOROTHIAZIDE:Herpes zoster:0.87), (AMINO ACIDSCARBOHYDRATESMINERALSSOYBEAN OILVITAMINS:Herpes zoster:0.08), (AMINO ACIDSDEXTROSEELECTROLYTES NOS:Herpes zoster:0.22), (AMISULPRIDE:Herpes zoster:0.41), (AMLODIPINE BESYLATEHYDROCHLOROTHIAZIDEOLMESARTAN MEDOXOMIL:Herpes zoster:0.09), (AMLODIPINE BESYLATEHYDROCHLOROTHIAZIDEVALSARTAN:Herpes zoster:0.39), (AMLODIPINE BESYLATEOLMESARTAN MEDOXOMIL:Herpes zoster:0.26), (AMLODIPINE BESYLATETELMISARTAN:Herpes zoster:1), (AMLODIPINEPERINDOPRIL:Herpes zoster:0.11), (AMLODIPINEVALSARTAN:Herpes zoster:0.78), (AMMONIUM GLYCYRRHIZATEGLYCINEMETHIONINE:Herpes zoster:0.31), (AMOXICILLINCLARITHROMYCINESOMEPRAZOLE:Herpes zoster:0.14), (AMPICILLIN SODIUM:Herpes zoster:0), (ANAGLIPTIN:Herpes zoster:0.65), (ANTIHISTAMINES NOS:Herpes zoster:0.24), (ATAZANAVIR:Herpes zoster:0.31), (ATENOLOLCHLORTHALIDONE:Herpes zoster:0.01), (ATROPINEDIPHENOXYLATE:Herpes zoster:0.09), (AZELASTINE HYDROCHLORIDEFLUTICASONE PROPIONATE:Herpes zoster:0.04), (BECLOMETHASONE:Herpes zoster:0.36), (BECLOMETHASONEFORMOTEROL:Herpes zoster:0.24), (BELATACEPT:Herpes zoster:0.3), (BENIDIPINE:Herpes zoster:0.02), (BENZBROMARONE:Herpes zoster:0.72), (BEPRIDIL:Herpes zoster:1.81), (BETAMETHASONE DIPROPIONATECALCIPOTRIENE:Herpes zoster:0.11), (BETAMETHASONE DIPROPIONATECALCIPOTRIENECALCIPOTRIENE HYDRATE:Herpes zoster:0.64), (BETAMETHASONE DIPROPIONATECLOTRIMAZOLE:Herpes zoster:0.35), (BETAMETHASONE SODIUM PHOSPHATENEOMYCIN SULFATE:Herpes zoster:0.25), (BETAMETHASONECALCIPOTRIENE:Herpes zoster:0.49), (BOCEPREVIR:Herpes zoster:0.15), (BUPRENORPHINE HYDROCHLORIDE:Herpes zoster:0.94), (CABAZITAXEL:Herpes zoster:0.06), (CALCITONIN:Herpes zoster:0.87), (CALCIUM CARBONATEERGOCALCIFEROLRETINOL:Herpes zoster:0.1), (CALCIUM CHLORIDEDEXTROSEPOTASSIUM CHLORIDESODIUM ACETATESODIUM CHLORIDE:Herpes zoster:0.53), (CALCIUMMAGNESIUM:Herpes zoster:0.76), (CALCIUMMAGNESIUMZINC:Herpes zoster:0.5), (CAMOSTAT MESYLATE:Herpes zoster:0.14), (CAMPHORMENTHOLMETHYL SALICYLATE:Herpes zoster:0.48), (CANAKINUMAB:Herpes zoster:1.09), (CARBAZOCHROME SODIUM SULFONATE:Herpes zoster:0.7), (CARBOXYMETHYLCELLULOSE SODIUM:Herpes zoster:0.29), (CARPERITIDE:Herpes zoster:0.03), (CARVEDILOL PHOSPHATE:Herpes zoster:1.84), (CEFOTAXIME SODIUM:Herpes zoster:0.87), (CEFOTAXIMECEFOTAXIME SODIUM:Herpes zoster:0.15), (CEFPODOXIME PROXETIL:Herpes zoster:0.25), (CEFUROXIME AXETIL:Herpes zoster:0.32), (CEPHARANTHINE:Herpes zoster:0.54), (CERITINIB:Herpes zoster:0.13), (CETIRIZINE HYDROCHLORIDEPSEUDOEPHEDRINE HYDROCHLORIDE:Herpes zoster:0.55), (CHLORPHENIRAMINEHYDROCODONE:Herpes zoster:0.46), (CLIKSTAR:Herpes zoster:0.03), (CLINDAMYCIN HYDROCHLORIDE:Herpes zoster:0.1), (COLISTIMETHATE SODIUM:Herpes zoster:0.02), (CYPROHEPTADINE HYDROCHLORIDE:Herpes zoster:0.13), (DABRAFENIB:Herpes zoster:0.37), (DACLIZUMAB:Herpes zoster:1.59), (DANTROLENE SODIUM:Herpes zoster:0.03), (DAPAGLIFLOZIN:Herpes zoster:0.19), (DEFLAZACORT:Herpes zoster:1.26), (DEGARELIX:Herpes zoster:0.28), (DEXTROAMPHETAMINE SULFATE:Herpes zoster:0.27), (DIMEMORFAN PHOSPHATE:Herpes zoster:0.08), (DIPYRONE:Herpes zoster:0.9), (DISOPYRAMIDE:Herpes zoster:0.12), (DOLASETRON MESYLATE:Herpes zoster:0.27), (DOLUTEGRAVIR:Herpes zoster:0.26), (DOPAMINE HYDROCHLORIDE:Herpes zoster:0), (DORZOLAMIDE HYDROCHLORIDE:Herpes zoster:0.58), (DUTASTERIDETAMSULOSIN HYDROCHLORIDE:Herpes zoster:0.66), (ECABET:Herpes zoster:0.04), (ECABET SODIUM:Herpes zoster:0.25), (ECONAZOLE:Herpes zoster:0.78), (ELBASVIRGRAZOPREVIR:Herpes zoster:0.12), (EMPAGLIFLOZINLINAGLIPTIN:Herpes zoster:0.61), (ENALAPRIL MALEATEHYDROCHLOROTHIAZIDE:Herpes zoster:0.04), (ESOMEPRAZOLE MAGNESIUMNAPROXEN:Herpes zoster:0.1), (ESTROGENS:Herpes zoster:0.34), (ESTRONESUS SCROFA OVARY:Herpes zoster:0.19), (ETRETINATE:Herpes zoster:1.88), (FAROPENEM SODIUM:Herpes zoster:0.6), (FENOFIBRIC ACID:Herpes zoster:0.52), (FERROUS SULFATEGLYCINE:Herpes zoster:0.06), (FLUOCINOLONE ACETONIDE:Herpes zoster:0.23), (FLUOCINONIDE:Herpes zoster:0.93), (FLUTICASONE PROPIONATEFORMOTEROL FUMARATE:Herpes zoster:0.01), (FONDAPARINUX SODIUM:Herpes zoster:0.21), (FORMOTEROL:Herpes zoster:0.79), (FORMOTEROL FUMARATEMOMETASONE FUROATE:Herpes zoster:0.96), (FOSFOMYCIN:Herpes zoster:0.03), (FOSFOMYCIN CALCIUM:Herpes zoster:0.08), (FOSINOPRIL:Herpes zoster:0.04), (FURSULTIAMINE:Herpes zoster:0.63), (GARLIC:Herpes zoster:0.34), (GIMERACILOTERACILTEGAFUR:Herpes zoster:0.87), (GLYCOPYRROLATEINDACATEROL MALEATE:Herpes zoster:0.36), (GLYCOPYRRONIUMINDACATEROL:Herpes zoster:1.43), (GUANABENZ ACETATE:Herpes zoster:1.08), (GUANFACINE:Herpes zoster:0.18), (HEPARIN CALCIUM:Herpes zoster:0.62), (HERBALSSAW PALMETTO:Herpes zoster:0.42), (HYDROCODONE BITARTRATEIBUPROFEN:Herpes zoster:0.42), (HYDROXYCHLOROQUINE DIPHOSPHATE:Herpes zoster:0.61), (IBRITUMOMAB TIUXETAN:Herpes zoster:0.23), (IFENPRODIL:Herpes zoster:0.11), (ILOPROST:Herpes zoster:0.63), (IMIGLUCERASE:Herpes zoster:0.21), (IMIPENEM:Herpes zoster:0.45), (IMMUNE GLOBULIN NOS:Herpes zoster:0.85), (INDACATEROL:Herpes zoster:0.74), (INDAPAMIDEPERINDOPRIL:Herpes zoster:1.57), (INFLIXIMAB-DYYB:Herpes zoster:0.29), (INTERFERON ALFA-2A:Herpes zoster:0.71), (INTERFERON ALFA-2B:Herpes zoster:0.47), (INTERFERON GAMMA-1B:Herpes zoster:0.21), (IODINE:Herpes zoster:0.39), (IPRAGLIFLOZIN L-PROLINE:Herpes zoster:0.12), (LABETALOL HYDROCHLORIDE:Herpes zoster:0.76), (LAPATINIB DITOSYLATE:Herpes zoster:0.26), (LENVATINIB:Herpes zoster:0.18), (LEVOCABASTINE HYDROCHLORIDE:Herpes zoster:0.79), (LEVOLEUCOVORIN CALCIUM:Herpes zoster:0.66), (LEVOMEFOLATE CALCIUMMETHYLCOBALAMINPYRIDOXAL PHOSPHATE ANHYDROUS:Herpes zoster:0.03), (LIOTHYRONINE:Herpes zoster:0.44), (LYSOZYME HYDROCHLORIDE:Herpes zoster:0.64), (MEGESTROL:Herpes zoster:0.35), (MEPOLIZUMAB:Herpes zoster:0.96), (METFORMIN HYDROCHLORIDESAXAGLIPTIN HYDROCHLORIDE:Herpes zoster:0.04), (METHENAMINE HIPPURATE:Herpes zoster:1.47), (METHENOLONE ENANTHATE:Herpes zoster:1.31), (METHOXY POLYETHYLENE GLYCOL-EPOETIN BETA:Herpes zoster:0.37), (MIDODRINE HYDROCHLORIDE:Herpes zoster:0.57), (MINOCYCLINEMINOCYCLINE HYDROCHLORIDE:Herpes zoster:1.77), (MITIGLINIDE CALCIUM HYDRATEVOGLIBOSE:Herpes zoster:0.42), (MOMETASONE:Herpes zoster:0), (MUPIROCINMUPIROCIN CALCIUM:Herpes zoster:0.88), (NADOLOL:Herpes zoster:0.85), (NALTREXONE:Herpes zoster:0.12), (NICARDIPINE:Herpes zoster:0.41), (NOREPINEPHRINE:Herpes zoster:0.07), (OLAPARIB:Herpes zoster:0), (OLODATEROL HYDROCHLORIDETIOTROPIUM BROMIDE MONOHYDRATE:Herpes zoster:1.52), (OLOPATADINE:Herpes zoster:0.2), (OXYBUTYNINOXYBUTYNIN CHLORIDE:Herpes zoster:0.24), (OXYMETAZOLINE:Herpes zoster:0.21), (PANITUMUMAB:Herpes zoster:0.48), (PARATHYROID HORMONE:Herpes zoster:0.14), (PARICALCITOL:Herpes zoster:0.42), (PATIROMER:Herpes zoster:0.52), (PEGINTERFERON ALFA-2B:Herpes zoster:0.27), (PEGINTERFERON BETA-1A:Herpes zoster:0.83), (PEGZEREPOETIN ALFA:Herpes zoster:0.15), (PEMETREXED:Herpes zoster:0.81), (PHENYTOIN:Herpes zoster:0.7), (PILOCARPINE HYDROCHLORIDE:Herpes zoster:0.16), (PIMECROLIMUS:Herpes zoster:0.33), (PIMOBENDAN:Herpes zoster:0.89), (POLYSTYRENE SULFONIC ACID:Herpes zoster:0.04), (POTASSIUM CHLORIDESODIUM CHLORIDESODIUM LACTATE:Herpes zoster:0.48), (PRANLUKAST:Herpes zoster:1.04), (PRAZOSIN HYDROCHLORIDE:Herpes zoster:0.56), (PREDNISOLONE SODIUM SUCCINATE:Herpes zoster:0.54), (PROBUCOL:Herpes zoster:0.11), (PROTRIPTYLINE HYDROCHLORIDE:Herpes zoster:0.13), (PYRIDOSTIGMINE BROMIDE:Herpes zoster:0.47), (QUINACRINE:Herpes zoster:0.39), (QUINAPRILQUINAPRIL HYDROCHLORIDE:Herpes zoster:0.38), (RALTEGRAVIR:Herpes zoster:0.51), (RAMUCIRUMAB:Herpes zoster:0.36), (REGORAFENIB:Herpes zoster:1.08), (RIZATRIPTAN:Herpes zoster:0.17), (SALMETEROL:Herpes zoster:0.74), (SITAFLOXACIN:Herpes zoster:0.08), (SODIUM FLUORIDE:Herpes zoster:0.9), (SODIUM PHOSPHATE, DIBASICSODIUM PHOSPHATE, MONOBASIC:Herpes zoster:0.4), (SUPLATAST TOSILATE:Herpes zoster:0.63), (TERBUTALINE:Herpes zoster:0.2), (TETRACYCLINE:Herpes zoster:1.12), (TIGECYCLINE:Herpes zoster:0.14), (TINZAPARIN:Herpes zoster:0.41), (TIPEPIDINE HIBENZATE:Herpes zoster:0.53), (TOLTERODINE:Herpes zoster:0.29), (TRAMETINIB:Herpes zoster:0.21), (TRANDOLAPRIL:Herpes zoster:0.41), (TRETINOIN:Herpes zoster:0.09), (TRIMIPRAMINE MALEATE:Herpes zoster:0.12), (TROSPIUM CHLORIDE:Herpes zoster:0.68), (TROXIPIDE:Herpes zoster:0.07), (VARDENAFIL HYDROCHLORIDE TRIHYDRATE:Herpes zoster:0.28), (VINORELBINE TARTRATE:Herpes zoster:0.23), (VONOPRAZAN:Herpes zoster:0.06), (ZILEUTON:Herpes zoster:0.42), (ZIPRASIDONE HYDROCHLORIDEZIPRASIDONE MESYLATE:Herpes zoster:0.01), (.ALPHA.1-PROTEINASE INHIBITOR HUMAN:Hiatus hernia:0.24), (ACARBOSE:Hiatus hernia:0.62), (ACETAMINOPHENOXYCODONEOXYCODONE HYDROCHLORIDE:Hiatus hernia:0.75), (ADO-TRASTUZUMAB EMTANSINE:Hiatus hernia:0.14), (AGALSIDASE BETA:Hiatus hernia:0.26), (ALBIGLUTIDE:Hiatus hernia:0.06), (ALEMTUZUMAB:Hiatus hernia:0.13), (ALISKIREN HEMIFUMARATE:Hiatus hernia:0.29), (AMANTADINE HYDROCHLORIDE:Hiatus hernia:0.28), (AMISULPRIDE:Hiatus hernia:0.26), (BENAZEPRILBENAZEPRIL HYDROCHLORIDE:Hiatus hernia:0.6), (BETAMETHASONE DIPROPIONATECALCIPOTRIENECALCIPOTRIENE HYDRATE:Hiatus hernia:0.2), (BETAMETHASONECALCIPOTRIENE:Hiatus hernia:0.08), (BISACODYL OR DOCUSATE SODIUM:Hiatus hernia:1.34), (BUPRENORPHINE HYDROCHLORIDENALOXONE HYDROCHLORIDE:Hiatus hernia:0.72), (CALCITONIN:Hiatus hernia:0.01), (CALCIUM ACETATE:Hiatus hernia:0.93), (CANAGLIFLOZINMETFORMIN HYDROCHLORIDE:Hiatus hernia:0.78), (CARVEDILOL PHOSPHATE:Hiatus hernia:1.29), (CEFTRIAXONE SODIUM:Hiatus hernia:0.53), (CETIRIZINE HYDROCHLORIDEPSEUDOEPHEDRINE HYDROCHLORIDE:Hiatus hernia:0.33), (CHONDROITIN SULFATE AGLUCOSAMINE:Hiatus hernia:0.01), (CLOBETASOL:Hiatus hernia:0.29), (CLONIDINE HYDROCHLORIDE:Hiatus hernia:0.44), (CODEINE PHOSPHATEGUAIFENESIN:Hiatus hernia:0.06), (DACLIZUMAB:Hiatus hernia:1.06), (DALTEPARIN SODIUM:Hiatus hernia:0.1), (DANAZOL:Hiatus hernia:0.39), (DARBEPOETIN ALFA:Hiatus hernia:0.7), (DASATINIB:Hiatus hernia:1.49), (DEXTROAMPHETAMINE SULFATE:Hiatus hernia:0.05), (DIGITOXIN:Hiatus hernia:0.02), (DIMENHYDRINATE:Hiatus hernia:0.67), (DIPYRONE:Hiatus hernia:0.26), (DORZOLAMIDE:Hiatus hernia:0.12), (DORZOLAMIDE HYDROCHLORIDE:Hiatus hernia:0.01), (DOXEPIN HYDROCHLORIDE:Hiatus hernia:0.5), (DUTASTERIDETAMSULOSIN HYDROCHLORIDE:Hiatus hernia:0.3), (ECONAZOLE:Hiatus hernia:0.22), (EDOXABAN TOSYLATE:Hiatus hernia:0.65), (EMPAGLIFLOZINLINAGLIPTIN:Hiatus hernia:1.15), (ENOXAPARIN:Hiatus hernia:1.42), (EPLERENONE:Hiatus hernia:0.64), (FENOTEROL HYDROBROMIDE:Hiatus hernia:0.03), (FENTANYL CITRATE:Hiatus hernia:0.01), (FLUOCINONIDE:Hiatus hernia:0.49), (FLUTICASONE:Hiatus hernia:1.1), (FORMOTEROL:Hiatus hernia:0.12), (FORMOTEROL FUMARATEMOMETASONE FUROATE:Hiatus hernia:0.67), (FUSIDIC ACID:Hiatus hernia:0.36), (HYDROCHLOROTHIAZIDETELMISARTAN:Hiatus hernia:0.68), (HYDROCODONE BITARTRATEIBUPROFEN:Hiatus hernia:0.22), (HYDROXYCHLOROQUINE DIPHOSPHATE:Hiatus hernia:0.38), (ILOPROST:Hiatus hernia:0.28), (IODINE:Hiatus hernia:0.09), (IPRATROPIUM:Hiatus hernia:1.4), (ISOSORBIDE DINITRATE:Hiatus hernia:0.09), (LACTASE:Hiatus hernia:0.13), (LEVOMEFOLATE CALCIUM:Hiatus hernia:0.18), (LIDOCAINEPRILOCAINE:Hiatus hernia:0.27), (LIOTHYRONINE:Hiatus hernia:0.15), (LITHIUM CARBONATE:Hiatus hernia:1.44), (LOMITAPIDE MESYLATE:Hiatus hernia:0.59), (MEGESTROL ACETATE:Hiatus hernia:0.45), (MEPERIDINE HYDROCHLORIDE:Hiatus hernia:0.1), (MEPOLIZUMAB:Hiatus hernia:0.47), (METFORMIN HYDROCHLORIDEVILDAGLIPTIN:Hiatus hernia:0.21), (METHYLPREDNISOLONE ACETATE:Hiatus hernia:1.19), (MIDODRINE:Hiatus hernia:0.29), (MOXIFLOXACIN HYDROCHLORIDE:Hiatus hernia:1.89), (MUPIROCINMUPIROCIN CALCIUM:Hiatus hernia:0.37), (NALOXONE HYDROCHLORIDEOXYCODONE HYDROCHLORIDE:Hiatus hernia:1.2), (NATEGLINIDE:Hiatus hernia:0.5), (NORETHINDRONE:Hiatus hernia:0), (OLMESARTAN:Hiatus hernia:0.13), (OXYBUTYNINOXYBUTYNIN CHLORIDE:Hiatus hernia:0.1), (PATIROMER:Hiatus hernia:0.18), (PEGINTERFERON BETA-1A:Hiatus hernia:0.86), (PEMETREXED DISODIUM:Hiatus hernia:0.26), (PENTOSAN POLYSULFATE SODIUM:Hiatus hernia:0.3), (PENTOXIFYLLINE:Hiatus hernia:0.18), (PONATINIB:Hiatus hernia:0.69), (PRAZOSIN HYDROCHLORIDE:Hiatus hernia:0.27), (PREDNISOLONE ACETATE:Hiatus hernia:0.86), (PREDNISOLONEPREDNISOLONE ACETATEPREDNISOLONE SODIUM SUCCINATE:Hiatus hernia:0.05), (SACCHAROMYCES CEREVISIAE:Hiatus hernia:0.18), (SACUBITRILVALSARTAN:Hiatus hernia:1.14), (SALMETEROL:Hiatus hernia:0.26), (SCOPOLAMINE:Hiatus hernia:0.91), (SILODOSIN:Hiatus hernia:0.05), (SITAGLIPTIN:Hiatus hernia:0.27), (SULFAMETHOXAZOLE:Hiatus hernia:0.68), (SULINDAC:Hiatus hernia:0.02), (TERAZOSIN HYDROCHLORIDE:Hiatus hernia:0.24), (THALIDOMIDE:Hiatus hernia:0.26), (TIMOLOL MALEATE:Hiatus hernia:0.37), (UMECLIDINIUM BROMIDE:Hiatus hernia:0.19), (VALGANCICLOVIR HYDROCHLORIDE:Hiatus hernia:0.39), (ZILEUTON:Hiatus hernia:0.1), (AMOXICILLIN:Hiccups:0.06), (ANASTROZOLE:Hiccups:0.31), (BUDESONIDE: Hiccups:0.57), (BUMETANIDE:Hiccups:0.75), (BUPROPION HYDROCHLORIDE:Hiccups:0.49), (CALCIUM CARBONATECHOLECALCIFEROL:Hiccups:0.24), (CERTOLIZUMAB PEGOL:Hiccups:0.35), (CETUXIMAB:Hiccups:0.47), (CLOZAPINE:Hiccups:0.04), (COLCHICI NE:Hiccups:0.05), (DASATINIB:Hiccups:0.15), (DILTIAZEM:Hiccups:0.44), (DIVALPROEX SODIUM:Hiccups:0.36), (EVOLOCUMAB:Hiccups:0.07), (FERROUS SULFATE:Hiccups:0.21), (HYDROCHLOROTHIAZIDELISINOPRIL:Hiccups:0.11), (IBRUTI NIB:Hiccups:0.6), (INSULIN DETEMIR:Hiccups:0.04), (LIDOCAINE:Hiccups:0.01), (NEBIVOLOL:Hiccups:0.07), (NILOTI NIB:Hiccups:0.16), (OXCARBAZEPINE:Hiccups:0.7), (OXYGEN:Hiccups:0.33), (PHENOBAR BITAL:Hiccups:0.2), (PRAVASTATIN SODIUM:Hiccups:0.38), (REGORAFENIB:Hiccups:0.1), (SACUBITRILVALSARTAN:Hiccups: 0.14), (TERIFLUNOMIDE:Hiccups:0.33), (THALIDOMIDE:Hiccups:0.69), (TOCILIZUMAB: Hiccups:0.09), (TRIAMCINOLONE ACETONIDE:Hiccups:0.09), (VALSARTAN:Hiccups:0.29), (VERAPAMIL HYDROCHLORIDE:Hiccups:0.12), (ANASTROZOLE:Hidradenitis:0.47),(ARMODAFINIL: Hidradenitis:0.48), (BISACODYL OR DOCUSATE SODIUM:Hidradenitis:0.15), (BISOPROLOL FUMARATE:Hidradenitis:0.33), (BUPRENORPHINE HYDROCHLORIDE:Hidradenitis:0.11), (CALCIUM CITRATE:Hidradenitis:0.09), (CANDESARTAN:Hidradenitis:0.05), (CANDESARTAN CILEXETILHYDROCHLOROTHIAZIDE:Hidradenitis:0.05), (CEFTRIAXONE SODIUM:Hidradenitis:0.09), (CLOTRIMAZOLE:Hidradenitis:0.16), (DEVICE:Hidradenitis:0.15), (ENOXAPARIN:Hidradenitis:0.91), (EVEROLIMUS:Hidradenitis:0.11), (EVOLOCUMAB: Hidradenitis:0.82), (HYDROXYCHLOROQUINE DIPHOSPHATE:Hidradenitis:0.21), (HYDROXYZINE PAMOATE:Hidradenitis:0.16), (IBRUTINIB:Hidradenitis:1.1), (ISOSORBIDE MONONITRATE:Hidradenitis:0.56), (LABETALOLLABETALOL HYDROCHLORIDE:Hidradenitis:0.12), (LERCANIDIPINE:Hidradenitis:0.2), (LERCANIDIPI NE HYDROCHLORIDE:Hidradenitis:0.03), (LOVASTATIN:Hidradenitis:0.9), (METHYLPREDNI SOLONE ACETATE:Hidradenitis:0.58), (NIFEDIPINE:Hidradenitis:0.19), (NILOTINIB:Hidradenitis:0.04), (NITROFURANTOIN:Hidradenitis:0.26), (NORETHINDRONE:Hidradenitis:0.01), (OXYGEN:Hidradenitis:0.19), (PANTOPRAZOLE MAGNESIUM:Hidradenitis:0.02), (PAROXETINEPAROXETINE HYDROCHLORIDE:Hidradenitis:0.21), (PENICILLIN:Hidradenitis:0.14), (PHENOBARBITAL:Hidradenitis:0.16), (POLYETHYLENE GLYCOLS:Hidradenitis:0.34), (PRAMIPEXOLE DIHYDROCHLORIDE:Hidradenitis:0.39), (PRAVASTATIN:Hidradenitis:0.77), (PROMETHA ZINE HYDROCHLORIDE:Hidradenitis:0.06), (RANOLAZINE:Hidradenitis:0.54), (SACUBITRILV ALSARTAN:Hidradenitis:0.2), (SODIUM CHLORIDE:Hidradenitis:0.17), (SODIUM OXYBATE:Hidradenitis:0.88), (SUMATRIPTANSUMATRIPTAN SUCCINATE:Hidradenitis:0.35), (TERIFLUNOMIDE:Hidradenitis:0.37), (TIOTROPIUM BROMIDE MONOHYDRATE:Hidradenitis:0.35), (BISOPROLOL FUMARATE:High density lipoprotein decreased:0.14), (DICLOFENAC SODIUM:High density lipoprotein decreased:0.32), (DULOXETINE HYDROCHLORIDE:High density lipoprotein decreased:0.06), (ENALAPRIL:High density lipoprotein decreased:0.26), (INSULIN ASPART:High density lipoprotein decreased:0.15), (WARFARIN:High density lipoprotein decreased:0.45), (AMPHETAMINE ASPARTATEAMPHETAMINE SULFATEDEXTROAMPHETAMINE SACCHARATEDEXTROAMPHETAMINE SULFATE:Hip arthroplasty:0.14), (BISOPROLOL FUMARATE:Hip arthroplasty:0.76), (BUDESONIDE:Hip arthroplasty:0.85), (CERTOLIZUMAB PEGOL:Hip arthroplasty:0.57), (CLOTRIMAZOLE:Hip arthroplasty:0.11), (DOXAZOSINDOXAZOSIN MESYLATE:Hip arthroplasty:0), (ERGOCALCIFEROL:Hip arthroplasty:0.13), (ESZOPICLONE:Hip arthroplasty:0.15), (EVOLOCUMAB:Hip arthroplasty:0.34), (FLECAINIDE:Hip arthroplasty:0.08), (GLIPIZIDE:Hip arthroplasty:0.35), (IBRUTINIB:Hip arthroplasty:1.29), (INSULIN HUMAN:Hip arthroplasty:0.24), (ISOSORBIDE MONONITRATE:Hip arthroplasty:0.32), (METHYLPREDNISOLONE SODIUM SUCCINATE:Hip arthroplasty:0.56), (MOXIFLOXACIN HYDROCHLORIDE:Hip arthroplasty:0.05), (NEBIVOLOL:Hip arthroplasty:0.5), (NIFEDIPINE:Hip arthroplasty:0.24), (OXYGEN:Hip arthroplasty:0.12), (PANTOPRAZOLE:Hip arthroplasty:0.82), (SACUBITRILVALSARTAN:Hip arthroplasty:0.25), (SOTALOL:Hip arthroplasty:0.17), (TERIFLUNOMIDE:Hip arthroplasty:0.1), (TOCILIZUMAB:Hip arthroplasty:0.11), (TRIAMCINOLONE ACETONIDE:Hip arthroplasty:0.24), (VERAPAMIL HYDROCHLORIDE:Hip arthroplasty:0.86), (VITAMIN B:Hip arthroplasty:0.11), (BLINATUMOMAB:Histiocytosis haematophagic:0.24), (DAUNORUBICIN:Histiocytosis haematophagic:0.08), (IBRITUMOMAB TIUXETAN:Histiocytosis haematophagic:0.24), (MITOXANTRONE HYDROCHLORIDE:Histiocytosis haematophagic:0.26), (PYRAZINAMIDE:Histoplasmosis:0.59), (PYRAZINAMIDE:HIV carrier:0.54), (ACARBOSE:HIV infection:0.89), (ACETAMINOPHENCAFFEINE:HIV infection:0.04), (ACETAMINOPHENPROPOXYPHENE HYDROCHLORIDE:HIV infection:0.41), (ACTIVATED CHARCOAL:HIV infection:0.21), (AGALSIDASE BETA:HIV infection:0.69), (ALBUTEROLIPRATROPIUM:HIV infection:0.12), (ALEMTUZUMAB:HIV infection:0.51), (ALISKIREN HEMIFUMARATE:HIV infection:0.87), (AMANTADINE:HIV infection:0.6), (AMANTADINE HYDROCHLORIDE:HIV infection:0.49), (AMLODIPINE BESYLATEATORVASTATIN CALCIUM:HIV infection:0.49), (AMLODIPINEVALSARTAN:HIV infection:0.86), (ANTIHEMOPHILIC FACTOR, HUMAN RECOMBINANT RESIDUES 743-1636 DELETED:HIV infection:0.01), (ANTIHISTAMINES NOS:HIV infection:0.24), (ASPIRINMAGNESIUM OXIDE:HIV infection:0.39), (AXITINIB:HIV infection:0.25), (AZITHROMYCIN OR METHOTREXATE SODIUM:HIV infection:0.24), (AZOSEMIDE:HIV infection:0.41), (BENDAMUSTINE:HIV infection:0.98), (BETAMETHASONE DIPROPIONATECALCIPOTRIENECALCIPOTRIENE HYDRATE:HIV infection:0.82), (BETAMETHASONECALCIPOTRIENE:HIV infection:0.35), (BETAXOLOL HYDROCHLORIDE:HIV infection:0.07), (BIFIDOBACTERIUM LONGUM SUBSP. INFANTIS:HIV infection:0.09), (BISACODYL OR DOCUSATE SODIUM:HIV infection:1.87), (BLINATUMOMAB:HIV infection:0.24), (BRENTUXIMAB VEDOTIN:HIV infection:0.56), (BRINZOLAMIDE:HIV infection:0.55), (BUPRENORPHINENALOXONE:HIV infection:0.07), (CALCIUM CARBONATEERGOCALCIFEROLRETINOL:HIV infection:0), (CALCIUM CITRATEVITAMIN D:HIV infection:0.12), (CALCIUM PANTOTHENATECYANOCOBALAMINNIACINAMIDEPYRIDOXINE HYDROCHLORIDERIBOFLAVINTHIAMINE MONONITRATE:HIV infection:0.11), (CALCIUMMAGNESIUM:HIV infection:0.33), (CALCIUMMAGNESIUMZINC:HIV infection:0.33), (CANAGLIFLOZINMETFORMIN HYDROCHLORIDE:HIV infection:0.51), (CAPSAICIN:HIV infection:0.69), (CARBIMAZOLE:HIV infection:0.44), (CARVEDILOL PHOSPHATE:HIV infection:1.38), (CASANTHRANOLDOCUSATE SODIUM:HIV infection:0.05), (CELIPROLOL:HIV infection:0.1), (CERTOLIZUMAB PEGOL:HIV infection:1.65), (CHLORAMBUCIL:HIV infection:0.43), (CHLORHEXIDINE:HIV infection:0.32), (CHLORMADINONE ACETATE:HIV infection:0.19), (CHONDROITIN SULFATE (CHICKEN):HIV infection:0.27), (CICLESONIDE:HIV infection:0.8), (CIDOFOVIR:HIV infection:0.66), (CLOMIPRAMINE:HIV infection:0.07), (CODEINE PHOSPHATEGUAIFENESIN:HIV infection:0), (COLISTIMETHATE SODIUM:HIV infection:0.26), (COLISTIN:HIV infection:0.17), (CORTICOTROPIN:HIV infection:0.38), (CRIZOTINIB:HIV infection:0.17), (DABRAFENIB:HIV infection:0.24), (DACLIZUMAB:HIV infection:1.29), (DANAZOL:HIV infection:0.92), (DAPAGLIFLOZIN:HIV infection:0.08), (DAPAGLIFLOZIN PROPANEDIOL:HIV infection:0.42), (DECITABINE:HIV infection:0.43), (DEFLAZACORT:HIV infection:0.51), (DESOXIMETASONE:HIV infection:0.16), (DIMETHICONESILICON DIOXIDESIMETHICONE:HIV infection:0), (DOFETILIDE:HIV infection:0.53), (DORZOLAMIDE:HIV infection:0.21), (DOXYLAMINE SUCCINATE:HIV infection:0.31), (DRONEDARONE:HIV infection:0.36), (DUTASTERIDETAMSULOSIN HYDROCHLORIDE:HIV infection:1.27), (ELETRIPTAN HYDROBROMIDE:HIV infection:1.81), (ENALAPRIL MALEATELERCANIDIPINE HYDROCHLORIDE:HIV infection:0.08), (ERTAPENEM SODIUM:HIV infection:0.31), (ETHINYL ESTRADIOLGESTODENE:HIV infection:0.01), (ETRETINATE:HIV infection:0.43), (EXEMESTANE:HIV infection:0.32), (FERROUS SULFATEGLYCINE:HIV infection:0.28), (FORMOTEROL FUMARATEMOMETASONE FUROATE:HIV infection:0.35), (FOSPHENYTOINFOSPHENYTOIN SODIUM:HIV infection:0.61), (GLYCOPYRROLATEINDACATEROL MALEATE:HIV infection:0.34), (GLYCOPYRRONIUMINDACATEROL:HIV infection:1.19), (GOSERELIN ACETATE:HIV infection:0.26), (GUANFACINE:HIV infection:0.95), (GUANFACINE HYDROCHLORIDE:HIV infection:0.87), (HYDROCHLOROTHIAZIDEQUINAPRIL HYDROCHLORIDE:HIV infection:0.29), (HYDROCORTISONE ACETATE:HIV infection:0.84), (HYOSCYAMINE:HIV infection:0.19), (IBRITUMOMAB TIUXETAN:HIV infection:0.87), (IDELALISIB:HIV infection:0.42), (IMIGLUCERASE:HIV infection:0.98), (IMMUNE GLOBULIN NOS:HIV infection:0.27), (INDAPAMIDEPERINDOPRIL:HIV infection:2.02), (INFLIXIMAB-DYYB: HIV infection:0.36), (INTERFERON GAMMA-1B:HIV infection:0.04), (IRINOTECAN HYDROCHLORIDE:HIV infection:0.32), (IXAZOMIB:HIV infection:0.46), (LACTOBACILLUS RHAMNOSUS:HIV infection:0.79), (LEVOMEFOLATE CALCIUM:HIV infection:0.11), (LEVOMEFOLATE CALCIUMMETHYLCOBALAMINPYRIDOXAL PHOSPHATE ANHYDROUS:HIV infection:0.08), (LEVOMILNACIPRAN HYDROCHLORIDE:HIV infection:0.12), (LEVOTHYROXINE SODIUMPOTASSIUM IODIDE:HIV infection:0.16), (LIDOCAINEPRILOCAINE:HIV infection:0.72), (LIOTHYRONINE:HIV infection:0.18), (LIOTHYRONINE SODIUM:HIV infection:0.39), (LOMITAPIDE MESYLATE:HIV infection:0.33), (LOMUSTINE:HIV infection:0.58), (LOVASTATIN:HIV infection:1.6), (LUTEIN:HIV infection:0.22), (MAGNESIUM ASPARTATE HYDROCHLORIDE:HIV infection:0.19), (METFORMIN PAMOATE:HIV infection:0.18), (METHENAMINE HIPPURATE:HIV infection:0.68), (METHENOLONE ENANTHATE:HIV infection:0.71), (METRELEPTIN:HIV infection:0.2), (MIDODRINE HYDROCHLORIDE:HIV infection:0.25), (MINOCYCLINEMINOCYCLINE HYDROCHLORIDE:HIV infection:2.96), (MIZORIBINE:HIV infection:0.16), (NADROPARIN CALCIUM:HIV infection:1.22), (NALBUPHINE HYDROCHLORIDE:HIV infection:0.31), (NATEGLINIDE:HIV infection:0.56), (NINTEDANIB:HIV infection:0.42), (NOREPINEPHRINE:HIV infection:0.37), (NORETHINDRONE:HIV infection:0.73), (OLODATEROL HYDROCHLORIDETIOTROPIUM BROMIDE MONOHYDRATE:HIV infection:0.87), (ONABOTULINUMTOXINA:HIV infection:1), (OSELTAMIVIR PHOSPHATE:HIV infection:0.55), (OSPEMIFENE:HIV infection:0.51), (PAMIDRONATE DISODIUM:HIV infection:0.53), (PANITUMUMAB:HIV infection:0.4), (PARICALCITOL:HIV infection:0.38), (PEGINTERFERON BETA-1A:HIV infection:0.14), (PEMETREXED DISODIUM:HIV infection:0.87), (PERPHENAZINE:HIV infection:0.35), (PHENOBARBITAL SODIUM:HIV infection:0.11), (PILOCARPINE:HIV infection:0.26), (PIMOBENDAN:HIV infection:0.01), (PONATINIB:HIV infection:1.13), (POSACONAZOLE:HIV infection:1.57), (PRANLUKAST:HIV infection:0.08), (PROPAFENONE HYDROCHLORIDE:HIV infection:0.56), (PSEUDOEPHEDRINE:HIV infection:0.32), (PSYLLIUM HUSK:HIV infection:0.47), (PYRIDOSTIGMINE:HIV infection:0.22), (QUINACRINE:HIV infection:0.07), (RETINOL:HIV infection:0.49), (RIVASTIGMINE:HIV infection:0.59), (RIVASTIGMINE TARTRATE:HIV infection:0.33), (RIZATRIPTAN:HIV infection:0.3), (RIZATRIPTAN BENZOATE:HIV infection:1.38), (SACCHAROMYCES CEREVISIAE:HIV infection:0.32), (SAPROPTERIN DIHYDROCHLORIDE:HIV infection:0.01), (SARPOGRELATE:HIV infection:0.28), (SODIUM PHOSPHATE, DIBASICSODIUM PHOSPHATE, MONOBASIC:HIV infection:0.04), (SOTALOL HYDROCHLORIDE:HIV infection:0.14), (SUNITINIB MALATE:HIV infection:0.48), (TAZOBACTAM:HIV infection:0.41), (TERIFLUNOMIDE:HIV infection:0.14), (TETRAZEPAM:HIV infection:0.08), (THEOPHYLLINE:HIV infection:0.04), (THYROID, PORCINETHYROID, UNSPECIFIED:HIV infection:0.06), (TIGECYCLINE:HIV infection:0.59), (TOCILIZUMAB:HIV infection:0.85), (TOCOPHEROL:HIV infection:0.88), (TOFACITINIB:HIV infection:0.15), (TRAMETINIB:HIV infection:0.11), (TRANDOLAPRIL:HIV infection:0.21), (TRIAMTERENE:HIV infection:0.37), (TROSPIUM CHLORIDE:HIV infection:0.68), (UMECLIDINIUM BROMIDE:HIV infection:0.44), (ZAFIRLUKAST:HIV infection:0.14), (ZILEUTON:HIV infection:0.16), (ATAZANAVIR:HIV infection CDC category C:0.24), (ATAZANAVIRRITONAVIR:HIV test positive:0.38), (COBICISTATELVITEGRAVIREMTRICITABINETENOFOVIR:HIV test positive:0.01), (EMTRICITABINETENOFOVIR:HIV test positive:0.04), (ABACAVIR SULFATELAMIVUDINE:Hodgkin's disease:0.04), (ADO-TRASTUZUMAB EMTANSINE:Hodgkin's disease:0.04), (ALFACALCIDOL:Hodgkin's disease:0.09), (ANASTROZOLE:Hodgkin's disease:1.04), (AZITHROMYCIN DIHYDRATE:Hodgkin's disease:0.03), (BICALUTAMIDE:Hodgkin's disease:0.02), (BISACODYL OR DOCUSATE SODIUM:Hodgkin's disease:0.11), (BUMETANIDE:Hodgkin's disease:0.57), (CANAKINUMAB:Hodgkin's disease:0.1), (CANDESARTAN:Hodgkin's disease:0.56), (CANDESARTAN CILEXETIL:Hodgkin's disease:0.65), (CARFILZOMIB:Hodgkin's disease:0.31), (CEFUROXIME:Hodgkin's disease:0.03), (CERTOLIZUMAB PEGOL:Hodgkin's disease:0.84), (CHOLESTYRAMINE:Hodgkin's disease:0.41), (CILASTATIN SODIUMIMIPENEM:Hodgkin's disease:0.5), (CLINDAMYCIN:Hodgkin's disease:0.25), (CLINDAMYCINCLINDAMYCIN PHOSPHATE:Hodgkin's disease:1.27), (CLONIDINE:Hodgkin's disease:0.52), (DANAZOL:Hodgkin's disease:0.06), (DEXLANSOPRAZOLE:Hodgkin's disease:1.19), (DILTIAZEM:Hodgkin's disease:0.78), (DOXAZOSIN MESYLATE:Hodgkin's disease:0.03), (DOXYCYCLINE:Hodgkin's disease:0.33), (DULOXETINE:Hodgkin's disease:0.07), (DUTASTERIDE:Hodgkin's disease:0.16), (ELETRIPTAN HYDROBROMIDE:Hodgkin's disease:0.03), (ENALAPRIL:Hodgkin's disease:1.47), (ENALAPRIL MALEATE:Hodgkin's disease:0.14), (EVOLOCUMAB:Hodgkin's disease:0.25), (EXEMESTANE:Hodgkin's disease:0.07), (EZETIMIBE:Hodgkin's disease:0.14), (EZETIMIBESIMVASTATIN:Hodgkin's disease:0.01), (FLECAINIDE:Hodgkin's disease:0.15), (HYDROCHLOROTHIAZIDELOSARTAN POTASSIUM:Hodgkin's disease:0.04), (IBRITUMOMAB TIUXETAN:Hodgkin's disease:0.44), (ILOPROST:Hodgkin's disease:0.02), (INDAPAMIDEPERINDOPRIL:Hodgkin's disease:0.18), (INDOMETHACIN:Hodgkin's disease:0.24), (LABETALOLLABETALOL HYDROCHLORIDE:Hodgkin's disease:0.38), (LEDIPASVIRSOFOSBUVIR:Hodgkin's disease:0.38), (LERCANIDIPINE:Hodgkin's disease:0.6), (LERCANIDIPINE HYDROCHLORIDE:Hodgkin's disease:0.21), (LETROZOLE:Hodgkin's disease:0.53), (MICAFUNGIN:Hodgkin's disease:0.02), (NALOXONE HYDROCHLORIDEOXYCODONE HYDROCHLORIDE:Hodgkin's disease:0.12), (PANTOPRAZOLE MAGNESIUM:Hodgkin's disease:0.07), (PAZOPANIB HYDROCHLORIDE:Hodgkin's disease:0.54), (PEMETREXED:Hodgkin's disease:0.08), (PEMETREXED DISODIUM:Hodgkin's disease:0.02), (PHENOBARBITAL:Hodgkin's disease:0.83), (PONATINIB:Hodgkin's disease:1.03), (QUETIAPINE:Hodgkin's disease:0.13), (RANOLAZINE:Hodgkin's disease:0.4), (RISEDRONATE SODIUM:Hodgkin's disease:0.41), (SECUKINUMAB:Hodgkin's disease:0.13), (SODIUM OXYBATE:Hodgkin's disease:0.31), (TETRACYCLINE:Hodgkin's disease:0.39), (TIZANIDINE HYDROCHLORIDE:Hodgkin's disease:0.28), (VARENICLINE TARTRATE:Hodgkin's disease:0.01), (VITAMIN B:Hodgkin's disease:0), (ZONISAMIDE:Hodgkin's disease:0.19), (ACETAMINOPHENBUTALBITALCAFFEINE:Hormone level abnormal:0.59), (ACETAMINOPHENCODEINE PHOSPHATE:Hormone level abnormal:0.68), (AMIODARONE:Hormone level abnormal:0), (BICALUTAMIDE:Hormone level abnormal:0.17), (BISACODYL OR DOCUSATE SODIUM:Hormone level abnormal:0.08), (BUDESONIDE:Hormone level abnormal:0.89), (CALCIUM CITRATE:Hormone level abnormal:0.68), (CARVEDILOL PHOSPHATE:Hormone level abnormal:0.16), (CODEINE:Hormone level abnormal:0.06), (DACLIZUMAB:Hormone level abnormal:0.18), (DIMENHYDRINATE:Hormone level abnormal:0.06), (DIPHENHYDRAMINE:Hormone level abnormal:0.34), (DROSPIRENONEETHINYL ESTRADIOLLEVOMEFOLATE CALCIUM:Hormone level abnormal:0.15), (ENALAPRIL:Hormone level abnormal:1.1), (EPINEPHRINE:Hormone level abnormal:0.15), (EVOLOCUMAB:Hormone level abnormal:0.67), (FLUCONAZOLE:Hormone level abnormal:0.05), (FLUTICASONE:Hormone level abnormal:0.12), (HYDROMORPHONE HYDROCHLORIDE:Hormone level abnormal:0.28), (HYDROXYCHLOROQUINE DIPHOSPHATE:Hormone level abnormal:0.16), (HYDROXYZINE PAMOATE:Hormone level abnormal:0.69), (HYOSCYAMINE:Hormone level abnormal:0.26), (IBRUTINIB:Hormone level abnormal:0.34), (INSULIN DETEMIR:Hormone level abnormal:0.03), (INSULIN LISPRO:Hormone level abnormal:0.3), (ISOPROPYL ALCOHOLLEUPROLIDE ACETATENORETHINDRONE ACETATE:Hormone level abnormal:0.51), (METHYLPREDNISOLONE ACETATE:Hormone level abnormal:0.18), (METHYLPREDNISOLONE SODIUM SUCCINATE:Hormone level abnormal:0.36), (METRONIDAZOLEMETRONIDAZOLE HYDROCHLORIDE:Hormone level abnormal:0.14), (OMEGA-3-ACID ETHYL ESTERS:Hormone level abnormal:0.17), (ONABOTULINUMTOXINA:Hormone level abnormal:0.01), (OXYBUTYNIN CHLORIDE:Hormone level abnormal:0.23), (PANTOPRAZOLE MAGNESIUM:Hormone level abnormal:0.13), (PENICILLIN:Hormone level abnormal:0.4), (PHENOBARBITAL:Hormone level abnormal:0.14), (PRAMIPEXOLE DIHYDROCHLORIDE:Hormone level abnormal:0.21), (QUETIAPINE:Hormone level abnormal:0.28), (RANOLAZINE:Hormone level abnormal:0.57), (TAMSULOSIN HYDROCHLORIDE:Hormone level abnormal:0.1), (TERIFLUNOMIDE:Hormone level abnormal:0.85), (TETRACYCLINE:Hormone level abnormal:0.21), (TRAMADOL HYDROCHLORIDE:Hormone level abnormal:0.23), (TRIAMCINOLONE ACETONIDE:Hormone level abnormal:0.51), (UMECLIDINIUM BROMIDEVILANTEROL TRIFENATATE:Hormone level abnormal:0.37), (ZINC:Hormone level abnormal:0.1), (ZONISAMIDE:Hormone level abnormal:0.19), (ABOBOTULINUMTOXINA:Hormone replacement therapy:0.22), (ACETAMINOPHENDIPHENHYDRAMINE HYDROCHLORIDE:Hormone replacement therapy:0.18), (ACITRETIN:Hormone replacement therapy:0.22), (AGALSIDASE BETA:Hormone replacement therapy:0.38), (ALBIGLUTIDE:Hormone replacement therapy:0.48), (ALCOHOL:Hormone replacement therapy:0.69), (ALEMTUZUMAB:Hormone replacement therapy:0.52), (ALIROCUMAB:Hormone replacement therapy:0.04), (AMINO ACIDSDEXTROSEELECTROLYTES NOS:Hormone replacement therapy:0.36), (AMISULPRIDE:Hormone replacement therapy:0.41), (AMLODIPINE BESYLATEHYDROCHLOROTHIAZIDEOLMESARTAN MEDOXOMIL:Hormone replacement therapy:0.36), (AMLODIPINE BESYLATEHYDROCHLOROTHIAZIDEVALSARTAN:Hormone replacement therapy:0.02), (AMOXICILLINCLAVULANIC ACID:Hormone replacement therapy:0.15), (ANHYDROUS CITRIC ACIDASPIRINSODIUM BICARBONATE:Hormone replacement therapy:0), (ANTIHISTAMINES NOS:Hormone replacement therapy:0.52), (APREPITANT:Hormone replacement therapy:0.45), (ARIPIPRAZOLE LAUROXIL:Hormone replacement therapy:0.55), (ATROPINEDIPHENOXYLATE:Hormone replacement therapy:0.1), (AXITINIB:Hormone replacement therapy:0.16), (BALSALAZIDE DISODIUM:Hormone replacement therapy:0.54), (BENDAMUSTINE HYDROCHLORIDE:Hormone replacement therapy:0.01), (BETAMETHASONE DIPROPIONATECALCIPOTRIENECALCIPOTRIENE HYDRATE:Hormone replacement therapy:0.95), (BETAMETHASONECALCIPOTRIENE:Hormone replacement therapy:0.56), (BETHANECHOL CHLORIDE:Hormone replacement therapy:0.01), (BICALUTAMIDE:Hormone replacement therapy:0.74), (BUPIVACAINE HYDROCHLORIDE:Hormone replacement therapy:0.07), (BUTYLSCOPOLAMINE BROMIDE:Hormone replacement therapy:0.75), (CANAKINUMAB:Hormone replacement therapy:0.89), (CARBIDOPA:Hormone replacement therapy:0.35), (CEFAZOLIN:Hormone replacement therapy:0.2), (CILASTATIN SODIUMIMIPENEM:Hormone replacement therapy:0.21), (CINACALCET HYDROCHLORIDE:Hormone replacement therapy:0.33), (CLOMIPRAMINE HYDROCHLORIDE:Hormone replacement therapy:0.44), (CODEINE PHOSPHATE:Hormone replacement therapy:0.16), (CRIZOTINIB:Hormone replacement therapy:0.13), (CYPROHEPTADINE:Hormone replacement therapy:1.04), (CYPROHEPTADINE HYDROCHLORIDE:Hormone replacement therapy:0.07), (DABIGATRAN:Hormone replacement therapy:0.33), (DANTROLENE SODIUM:Hormone replacement therapy:0.13), (DIENOGEST:Hormone replacement therapy:0.65), (DIPYRIDAMOLE:Hormone replacement therapy:0.44), (DIPYRONEPROMETHAZINE HYDROCHLORIDE:Hormone replacement therapy:0.34), (DOLASETRON MESYLATE:Hormone replacement therapy:0.16), (DUTASTERIDETAMSULOSIN HYDROCHLORIDE:Hormone replacement therapy:0.36), (ECONAZOLE:Hormone replacement therapy:0.37), (EMPAGLIFLOZINLINAGLIPTIN:Hormone replacement therapy:1.15), (ETHINYL ESTRADIOLFERROUS FUMARATENORETHINDRONE:Hormone replacement therapy:0.2), (ETHINYL ESTRADIOLFERROUS FUMARATENORETHINDRONE ACETATE:Hormone replacement therapy:0.1), (EVEROLIMUS:Hormone replacement therapy:1.41), (FAMCICLOVIR:Hormone replacement therapy:0.06), (FERROUS BISGLYCINATE:Hormone replacement therapy:0.26), (FERROUS FUMARATE:Hormone replacement therapy:0.04), (FLUOCINOLONE ACETONIDE:Hormone replacement therapy:0.18), (FORMOTEROL FUMARATEMOMETASONE FUROATE:Hormone replacement therapy:0.72), (FULVESTRANT:Hormone replacement therapy:0.52), (GLIPIZIDEMETFORMIN HYDROCHLORIDE:Hormone replacement therapy:0.13), (GLYCOPYRROLATEINDACATEROL MALEATE:Hormone replacement therapy:0.07), (GLYCOPYRRONIUM:Hormone replacement therapy:0.15), (GLYCOPYRRONIUMINDACATEROL:Hormone replacement therapy:0.63), (GUANFACINE HYDROCHLORIDE:Hormone replacement therapy:0.57), (HORSE CHESTNUT:Hormone replacement therapy:0.03), (HYALURONIC ACID:Hormone replacement therapy:0.21), (HYDROCHLOROTHIAZIDETELMISARTAN:Hormone replacement therapy:0.8), (HYDROCODONE BITARTRATEIBUPROFEN:Hormone replacement therapy:0.83), (HYDROXYCHLOROQUINE DIPHOSPHATE:Hormone replacement therapy:0.87), (ILOPROST:Hormone replacement therapy:0.29), (INFLIXIMAB-DYYB:Hormone replacement therapy:0.29), (INSULIN DEGLUDEC:Hormone replacement therapy:0.64), (INTERFERON:Hormone replacement therapy:0.08), (IRINOTECAN:Hormone replacement therapy:0.23), (IRON SUCROSE:Hormone replacement therapy:0.06), (LACOSAMIDE:Hormone replacement therapy:0.19), (LACTASE:Hormone replacement therapy:0.72), (LANREOTIDE ACETATE:Hormone replacement therapy:1.08), (LAPATINIB DITOSYLATE:Hormone replacement therapy:0.06), (LERCANIDIPINE:Hormone replacement therapy:0.87), (LERCANIDIPINE HYDROCHLORIDE:Hormone replacement therapy:0.57), (MEPOLIZUMAB:Hormone replacement therapy:0.55), (METAMIZOLE:Hormone replacement therapy:0.18), (METHYLPREDNISOLONE ACETATE:Hormone replacement therapy:1.73), (MIDODRINE:Hormone replacement therapy:0.6), (MIDODRINE HYDROCHLORIDE:Hormone replacement therapy:0.4), (NALOXONE:Hormone replacement therapy:0.2), (NALOXONE HYDROCHLORIDEOXYCODONE HYDROCHLORIDE:Hormone replacement therapy:1.15), (NALOXONE HYDROCHLORIDETILIDINE HYDROCHLORIDE:Hormone replacement therapy:0.35), (NAPROXEN SODIUMSUMATRIPTAN SUCCINATE:Hormone replacement therapy:0.13), (NATEGLINIDE:Hormone replacement therapy:0.56), (NILOTINIB:Hormone replacement therapy:2.18), (NIRAPARIB:Hormone replacement therapy:0.41), (OLODATEROL HYDROCHLORIDETIOTROPIUM BROMIDE MONOHYDRATE:Hormone replacement therapy:0.75), (OXAPROZIN:Hormone replacement therapy:0.18), (OXYBUTYNINOXYBUTYNIN CHLORIDE:Hormone replacement therapy:0.48), (PALIPERIDONE PALMITATE:Hormone replacement therapy:0.61), (PAMIDRONATE DISODIUM:Hormone replacement therapy:0.77), (PANTOPRAZOLE MAGNESIUM:Hormone replacement therapy:1.93), (PAPAYA:Hormone replacement therapy:0.01), (PARATHYROID HORMONE:Hormone replacement therapy:0.03), (PASIREOTIDE:Hormone replacement therapy:0.33), (PATIROMER:Hormone replacement therapy:0.15), (PAZOPANIB HYDROCHLORIDE:Hormone replacement therapy:1.17), (PENICILLIN G:Hormone replacement therapy:0.28), (PERTUZUMAB:Hormone replacement therapy:0.03), (PHENAZOPYRIDINE HYDROCHLORIDE:Hormone replacement therapy:0.12), (PNEUMOCOCCAL VACCINE, POLYVALENT 23:Hormone replacement therapy:0.22), (PONATINIB:Hormone replacement therapy:1.21), (PRAZOSIN:Hormone replacement therapy:1.15), (PRAZOSIN HYDROCHLORIDE:Hormone replacement therapy:0.91), (PREDNISOLONE ACETATE:Hormone replacement therapy:1.07), (PREDNISOLONE SODIUM PHOSPHATE:Hormone replacement therapy:0.25), (PROTRIPTYLINE HYDROCHLORIDE:Hormone replacement therapy:0.92), (PYRAZINAMIDE:Hormone replacement therapy:0.76), (QUINACRINE:Hormone replacement therapy:0.29), (RADIATION THERAPY:Hormone replacement therapy:0.01), (SALMETEROL XINAFOATE:Hormone replacement therapy:0.55), (SAXAGLIPTIN HYDROCHLORIDE:Hormone replacement therapy:0.13), (SIBUTRAMINE HYDROCHLORIDE:Hormone replacement therapy:0.25), (SOFOSBUVIRVELPATASVIR:Hormone replacement therapy:0.08), (TETRACYCLINE:Hormone replacement therapy:1.44), (THEOPHYLLINE:Hormone replacement therapy:0), (TRIMIPRAMINE MALEATE:Hormone replacement therapy:0.01), (TRIPTORELIN PAMOATE:Hormone replacement therapy:0.18), (UMECLIDINIUM BROMIDEVILANTEROL TRIFENATATE:Hormone replacement therapy:1.26), (VALGANCICLOVIR HYDROCHLORIDE:Hormone replacement therapy:0.49), (VINCRISTINE SULFATE:Hormone replacement therapy:0.1), (YEAST:Hormone replacement therapy:0.34), (ZILEUTON:Hormone replacement therapy:0.37), (ACARBOSE:Hormone therapy:0.15), (ACETAMINOPHENOXYCODONE HYDROCHLORIDEOXYCODONE TEREPHTHALATE:Hormone therapy:0.01), (ACETAMINOPHENTRAMADOL:Hormone therapy:0.02), (ACETAZOLAMIDE:Hormone therapy:0.5), (ALCOHOL:Hormone therapy:0.38), (ALENDRONIC ACID:Hormone therapy:0.14), (AMLODIPINE BESYLATEVALSARTAN:Hormone therapy:0.17), (AZELASTINE:Hormone therapy:0.06), (AZITHROMYCIN DIHYDRATE:Hormone therapy:0.3), (BORTEZOMIB:Hormone therapy:0.42), (BRINZOLAMIDE:Hormone therapy:0.22), (BUPRENORPHINE HYDROCHLORIDENALOXONE HYDROCHLORIDE:Hormone therapy:0.36), (CALCITONIN SALMON:Hormone therapy:0.05), (CALCIUM ACETATE:Hormone therapy:0.24), (CALCIUM CARBONATEVITAMIN D:Hormone therapy:0.27), (CANAGLIFLOZINMETFORMIN HYDROCHLORIDE:Hormone therapy:0.15), (CANAKINUMAB:Hormone therapy:0.35), (CANDESARTAN CILEXETIL:Hormone therapy:0.84), (CANDESARTAN CILEXETILHYDROCHLOROTHIAZIDE:Hormone therapy:0.8), (CARVEDILOL PHOSPHATE:Hormone therapy:1.08), (CETRORELIX ACETATE:Hormone therapy:0.39), (CHLORDIAZEPOXIDE HYDROCHLORIDECLIDINIUM BROMIDE:Hormone therapy:0.06), (CHONDROITIN SULFATE (BOVINE)GLUCOSAMINE HYDROCHLORIDE:Hormone therapy:0.22), (CLOZAPINE:Hormone therapy:0.57), (COD LIVER OIL:Hormone therapy:0.21), (CODEINE:Hormone therapy:0.4), (CORTISONEHYDROCORTISONE:Hormone therapy:1.19), (DACLIZUMAB:Hormone therapy:0.74), (DASATINIB:Hormone therapy:1.28), (DEVICE:Hormone therapy:0.7), (DIENOGEST:Hormone therapy:0.29), (DOXAZOSINDOXAZOSIN MESYLATE:Hormone therapy:0.36), (EMPAGLIFLOZINLINAGLIPTIN:Hormone therapy:0.37), (ENALAPRIL MALEATE:Hormone therapy:1.13), (ENOXAPARIN:Hormone therapy:0.79), (ERYTHROPOIETIN:Hormone therapy:0.22), (ETORICOXIB:Hormone therapy:0.23), (EVOLOCUMAB:Hormone therapy:1.32), (FENTANYL CITRATE:Hormone therapy:0.14), (FLECAINIDE:Hormone therapy:0.57), (FLUTICASONE FUROATEVILANTEROL TRIFENATATE:Hormone therapy:0.35), (FLUTICASONEFLUTICASONE PROPIONATE:Hormone therapy:0.6), (FORMOTEROL FUMARATEMOMETASONE FUROATE:Hormone therapy:0.05), (GLICLAZIDE:Hormone therapy:0.52), (GLYCOPYRRONIUMINDACATEROL:Hormone therapy:0.02), (HEPARIN SODIUM:Hormone therapy:0.04), (HYDROMORPHONE:Hormone therapy:0.45), (HYDROXYCHLOROQUINE DIPHOSPHATE:Hormone therapy:0.25), (HYDROXYUREA:Hormone therapy:0.03), (IBRUTINIB:Hormone therapy:0.54), (INDAPAMIDEPERINDOPRIL:Hormone therapy:0.06), (INFLUENZA VIRUS VACCINE:Hormone therapy:0.48), (INSULIN DEGLUDEC:Hormone therapy:0.01), (IPRATROPIUM:Hormone therapy:0.57), (LABETALOLLABETALOL HYDROCHLORIDE:Hormone therapy:0.48), (LACOSAMIDE:Hormone therapy:0.26), (LAPATINIB DITOSYLATE:Hormone therapy:0.24), (LEDIPASVIRSOFOSBUVIR:Hormone therapy:0.31), (LEVOMEFOLATE CALCIUMMETHYLCOBALAMINPYRIDOXAL PHOSPHATE ANHYDROUS:Hormone therapy:0.08), (LIDOCAINEPRILOCAINE:Hormone therapy:0.14), (LITHIUM:Hormone therapy:0.05), (LOMITAPIDE MESYLATE:Hormone therapy:0.01), (MESNA:Hormone therapy:0.07), (METHENAMINE HIPPURATE:Hormone therapy:0.25), (METHYLPREDNISOLONE ACETATE:Hormone therapy:0.6), (MICONAZOLE NITRATE:Hormone therapy:0.07), (NALOXONE HYDROCHLORIDEOXYCODONE HYDROCHLORIDE:Hormone therapy:0.13), (NORTRIPTYLINE HYDROCHLORIDE:Hormone therapy:0.14), (OCTREOTIDE:Hormone therapy:0.01), (PANTOPRAZOLE MAGNESIUM:Hormone therapy:0.89), (PAZOPANIB HYDROCHLORIDE:Hormone therapy:0.87), (PEGINTERFERON BETA-1A:Hormone therapy:0.64), (PONATINIB:Hormone therapy:0.26), (PRAZOSIN HYDROCHLORIDE:Hormone therapy:0.21), (PREDNISOLONE ACETATE:Hormone therapy:0.07), (PYRIDOXINE:Hormone therapy:0.32), (ROFLUMILAST:Hormone therapy:0.55), (SACUBITRILVALSARTAN:Hormone therapy:0.75), (SECUKINUMAB:Hormone therapy:0.62), (SENNA LEAFSENNOSIDESSENNOSIDES A AND B:Hormone therapy:0.21), (SOTALOL:Hormone therapy:0.59), (SUMATRIPTANSUMATRIPTAN SUCCINATE:Hormone therapy:1.57), (TETRACYCLINE:Hormone therapy:0.46), (TRIAMCINOLONE:Hormone therapy:0.26), (UMECLIDINIUM BROMIDEVILANTEROL TRIFENATATE:Hormone therapy:0.52), (ZOPICLONE:Hormone therapy:0.42), (ACENOCOUMAROL:Hormone-refractory prostate cancer:1.21), (ACETAMINOPHENBUTALBITALCAFFEINE:Hormone-refractory prostate cancer:0.86), (ACETAMINOPHENOXYCODONEOXYCODONE HYDROCHLORIDE:Hormone-refractory prostate cancer:0.29), (ACYCLOVIR:Hormone-refractory prostate cancer:0.27), (ADO-TRASTUZUMAB EMTANSINE:Hormone-refractory prostate cancer:0.39), (AGALSIDASE BETA:Hormone-refractory prostate cancer:0.16), (ALISKIREN HEMIFUMARATE:Hormone-refractory prostate cancer:0.4), (AMLODIPINE BESYLATEHYDROCHLOROTHIAZIDEOLMESARTAN MEDOXOMIL:Hormone-refractory prostate cancer:0), (AMLODIPINEVALSARTAN:Hormone-refractory prostate cancer:0.02), (ANASTROZOLE:Hormone-refractory prostate cancer:1.46),(ARMODAFINIL:Hormone-refractory prostate cancer:0.65), (BENAZEPRILBENAZEPRIL HYDROCHLORIDE:Hormone-refractory prostate cancer:0.45), (BISACODYL OR DOCUSATE SODIUM:Hormone-refractory prostate cancer:1.27), (BROMAZEPAM:Hormone-refractory prostate cancer:0.68), (CALCIUM ACETATE:Hormone-refractory prostate cancer:0.81), (CARBIMAZOLE:Hormone-refractory prostate cancer:0.21), (CARFILZOMIB:Hormone-refractory prostate cancer:0.66), (CARISOPRODOL:Hormone-refractory prostate cancer:0.21), (CARVEDILOL PHOSPHATE:Hormone-refractory prostate cancer:1.38), (CLOTRIMAZOLE:Hormone-refractory prostate cancer:0.5), (CLOZAPINE:Hormone-refractory prostate cancer:0.31), (CYCLOBENZAPRINE:Hormone-refractory prostate cancer:0.41), (DANAZOL:Hormone-refractory prostate cancer:0.06), (DAPAGLIFLOZIN PROPANEDIOL:Hormone-refractory prostate cancer:0.01), (DASATINIB:Hormone-refractory prostate cancer:1.02), (DEFLAZACORT:Hormone-refractory prostate cancer:0.18), (DESLORATADINE:Hormone-refractory prostate cancer:0.51), (DEVICE:Hormone-refractory prostate cancer:0.01), (DEXAMETHASONE ACETATE:Hormone-refractory prostate cancer:0.16), (DOXEPIN:Hormone-refractory prostate cancer:0.06), (DOXEPIN HYDROCHLORIDE:Hormone-refractory prostate cancer:0.1), (DRONABINOL:Hormone-refractory prostate cancer:0.36), (DUTASTERIDETAMSULOSIN HYDROCHLORIDE:Hormone-refractory prostate cancer:0.28), (ELETRIPTAN HYDROBROMIDE:Hormone-refractory prostate cancer:0.41), (EPINEPHRINE:Hormone-refractory prostate cancer:0.43), (ESTRADIOL:Hormone-refractory prostate cancer:0.05), (ESTROGENS, CONJUGATEDMEDROXYPROGESTERONE ACETATE:Hormone-refractory prostate cancer:0.14), (EXEMESTANE:Hormone-refractory prostate cancer:0.21), (FENOFIBRIC ACID:Hormone-refractory prostate cancer:0.2), (FLUTICASONE FUROATESALMETEROL XINAFOATE:Hormone-refractory prostate cancer:0.27), (FLUTICASONEFLUTICASONE PROPIONATE:Hormone-refractory prostate cancer:0.5), (FORMOTEROL FUMARATE DIHYDRATEMOMETASONE FUROATE:Hormone-refractory prostate cancer:0.07), (FULVESTRANT:Hormone-refractory prostate cancer:0.05), (FURSULTIAMINE:Hormone-refractory prostate cancer:0.04), (GUAIFENESIN:Hormone-refractory prostate cancer:0.33), (HEPARIN CALCIUM:Hormone-refractory prostate cancer:0.23), (HERBALSTURMERIC:Hormone-refractory prostate cancer:0.11), (IBRUTINIB:Hormone-refractory prostate cancer:0.78), (ILOPROST:Hormone-refractory prostate cancer:0.49), (IMATINIB MESYLATE:Hormone-refractory prostate cancer:0.14), (INVESTIGATIONAL PRODUCT:Hormone-refractory prostate cancer:0.23), (LEDIPASVIRSOFOSBUVIR:Hormone-refractory prostate cancer:0.19), (LEVALBUTEROL HYDROCHLORIDE:Hormone-refractory prostate cancer:1.04), (LIDOCAINE HYDROCHLORIDE:Hormone-refractory prostate cancer:1.16), (LISDEXAMFETAMINE DIMESYLATE:Hormone-refractory prostate cancer:0.34), (LITHIUM CARBONATE:Hormone-refractory prostate cancer:0.79), (MECLIZINEMECLIZINE HYDROCHLORIDE:Hormone-refractory prostate cancer:0.02), (METAXALONE:Hormone-refractory prostate cancer:0.62), (METHENAMINE HIPPURATE:Hormone-refractory prostate cancer:0.19), (METHYLPREDNISOLONE ACETATE:Hormone-refractory prostate cancer:0.11), (MINOCYCLINEMINOCYCLINE HYDROCHLORIDE:Hormone-refractory prostate cancer:0.02), (MOXIFLOXACIN HYDROCHLORIDE:Hormone-refractory prostate cancer:0.95), (MOXONIDINE:Hormone-refractory prostate cancer:0.1), (MYCOPHENOLATE MOFETILMYCOPHENOLATE MOFETIL HYDROCHLORIDE:Hormone-refractory prostate cancer:0.17), (NABUMETONE:Hormone-refractory prostate cancer:0.22), (NADOLOL:Hormone-refractory prostate cancer:0.12), (NATEGLINIDE:Hormone-refractory prostate cancer:0.16), (NILOTINIB:Hormone-refractory prostate cancer:1.09), (NINTEDANIB:Hormone-refractory prostate cancer:0.41), (OMEPRAZOLE SODIUM:Hormone-refractory prostate cancer:0.12), (OXCARBAZEPINE:Hormone-refractory prostate cancer:1.12), (PANTOPRAZOLE MAGNESIUM:Hormone-refractory prostate cancer:0.52), (PAROXETINEPAROXETINE HYDROCHLORIDE:Hormone-refractory prostate cancer:0.39), (PATIROMER:Hormone-refractory prostate cancer:0.08), (PEMETREXED DISODIUM:Hormone-refractory prostate cancer:0.46), (PENICILLIN:Hormone-refractory prostate cancer:0.57), (PENTOXIFYLLINE:Hormone-refractory prostate cancer:0.07), (PHENOBARBITAL:Hormone-refractory prostate cancer:0.36), (PHENYTOIN SODIUM:Hormone-refractory prostate cancer:0.29), (PONATINIB:Hormone-refractory prostate cancer:0.25), (PRIMIDONE:Hormone-refractory prostate cancer:0.84), (PROPAFENONE:Hormone-refractory prostate cancer:0.24), (PROPRANOLOLPROPRANOLOL HYDROCHLORIDE:Hormone-refractory prostate cancer:0.1), (PYRIDOXINE:Hormone-refractory prostate cancer:0.12), (RALOXIFENE HYDROCHLORIDE:Hormone-refractory prostate cancer:0.27), (RIZATRIPTAN BENZOATE:Hormone-refractory prostate cancer:0.27), (ROFLUMILAST:Hormone-refractory prostate cancer:0.4), (SACUBITRILVALSARTAN:Hormone-refractory prostate cancer:0.49), (SEVELAMER HYDROCHLORIDE:Hormone-refractory prostate cancer:0.34), (SODIUM OXYBATE:Hormone-refractory prostate cancer:0.92), (SUMATRIPTANSUMATRIPTAN SUCCINATE:Hormone-refractory prostate cancer:0.32), (SUNITINIB MALATE:Hormone-refractory prostate cancer:0.09), (TERIFLUNOMIDE:Hormone-refractory prostate cancer:0.15), (TETRACYCLINE:Hormone-refractory prostate cancer:0.24), (THYROID, PORCINE:Hormone-refractory prostate cancer:0.22), (TIOTROPIUM:Hormone-refractory prostate cancer:0.54), (TOPIRAMATE:Hormone-refractory prostate cancer:1.61), (UMECLIDINIUM BROMIDEVILANTEROL TRIFENATATE:Hormone-refractory prostate cancer:0.26), (VARENICLINE TARTRATE:Hormone-refractory prostate cancer:0.87), (VILAZODONE HYDROCHLORIDE:Hormone-refractory prostate cancer:0.01), (ACARBOSE:Hot flush:0.06), (ACETAMINOPHENOXYCODONEOXYCODONE HYDROCHLORIDE:Hot flush:0.55), (ALBIGLUTIDE:Hot flush:0.17), (ALCOHOL:Hot flush:0.49), (ALENDRONIC ACID:Hot flush:0.42), (AMIODARONE HYDROCHLORIDE:Hot flush:0.51), (AMISULPRIDE:Hot flush:0.06), (AMLODIPINE BESYLATEHYDROCHLOROTHIAZIDEOLMESARTAN MEDOXOMIL:Hot flush:0.02), (AZELASTINE:Hot flush:0.14), (BISACODYL OR DOCUSATE SODIUM:Hot flush:1.16), (BUTYLSCOPOLAMINE BROMIDE:Hot flush:0.08), (CALCIUMMAGNESIUM:Hot flush:0.15), (CANAGLIFLOZINMETFORMIN HYDROCHLORIDE:Hot flush:0.31), (CANAKINUMAB:Hot flush:0.14), (CAPSAICIN:Hot flush:0.06), (CAPTOPRIL:Hot flush:0.37), (CLINDAMYCINCLINDAMYCIN PHOSPHATE:Hot flush:1.07), (CORTICOTROPIN:Hot flush:0.4), (DACLIZUMAB:Hot flush:0.85), (DANAZOL:Hot flush:0.1), (DESONIDE:Hot flush:0), (DIMETHICONE:Hot flush:0.03), (DOXAZOSINDOXAZOSIN MESYLATE:Hot flush:0.44), (DRONABINOL:Hot flush:1.23), (EMPAGLIFLOZINLINAGLIPTIN:Hot flush:0.36), (ERYTHROPOIETIN:Hot flush:0.44), (ESOMEPRAZOLE MAGNESIUMNAPROXEN:Hot flush:0.14), (ETHINYL ESTRADIOLNORETHINDRONE:Hot flush:0.1), (FEBUXOSTAT:Hot flush:0.22), (FENTANYL CITRATE:Hot flush:0.04), (FISH OIL\OMEGA-3 FATTY ACIDS:Hot flush:0.64), (FLECAINIDE:Hot flush:0.56), (FLUDROCORTISONE ACETATE:Hot flush:0.21), (GUANFACINE:Hot flush:0.02), (HYDROCHLOROTHIAZIDETELMISARTAN:Hot flush:0.32), (HYDROCODONE BITARTRATEIBUPROFEN:Hot flush:0.23), (HYDROCORTISONE ACETATE:Hot flush:0.08), (HYDROXYCHLOROQUINE DIPHOSPHATE:Hot flush:0.45), (IMATINIB MESYLATE:Hot flush:0.81), (IMIPRAMINE HYDROCHLORIDE:Hot flush:0.68), (INFLUENZA VIRUS VACCINE:Hot flush:0.39), (INSULIN GLULISINE:Hot flush:0.13), (LAPATINIB DITOSYLATE:Hot flush:0.26), (LEDIPASVIRSOFOSBUVIR:Hot flush:0.29), (LERCANIDIPINE:Hot flush:0.04), (LERCANIDIPINE HYDROCHLORIDE:Hot flush:0.15), (LIDOCAINE HYDROCHLORIDE:Hot flush:0.46), (LOMITAPIDE MESYLATE:Hot flush:0.4), (LOPERAMIDE:Hot flush:0.05), (MEFENAMIC ACID:Hot flush:0.05), (METHIMAZOLE:Hot flush:0.13), (METHYLPREDNISOLONE ACETATE:Hot flush:0.78), (MOXIFLOXACIN HYDROCHLORIDE:Hot flush:0.97), (NALOXONE HYDROCHLORIDEOXYCODONE HYDROCHLORIDE:Hot flush:0.19), (NALTREXONE:Hot flush:0.17), (NEBIVOLOL:Hot flush:0.03), (PANTOPRAZOLE MAGNESIUM:Hot flush:0.77), (PAZOPANIB HYDROCHLORIDE:Hot flush:1.19), (PEGINTERFERON BETA-1A:Hot flush:0.96), (PENICILLIN:Hot flush:0.76), (PIROXICAM:Hot flush:0.26), (PONATINIB:Hot flush:0.45), (PRASTERONE:Hot flush:0), (PRAZOSIN:Hot flush:0.28), (PRAZOSIN HYDROCHLORIDE:Hot flush:0.2), (PROPAFENONE HYDROCHLORIDE:Hot flush:0.04), (PROTRIPTYLINE HYDROCHLORIDE:Hot flush:0), (RABEPRAZOLE:Hot flush:0.46), (RIVASTIGMINE:Hot flush:0.09), (ROFLUMILAST:Hot flush:0.41), (TERBINAFINE HYDROCHLORIDE:Hot flush:0.15), (TERBUTALINE SULFATE:Hot flush:0.07), (TETRACYCLINE:Hot flush:0.94), (THALIDOMIDE:Hot flush:0.56), (TRIAMCINOLONE:Hot flush:0.33), (UMECLIDINIUM BROMIDEVILANTEROL TRIFENATATE:Hot flush:0.9), (VINORELBINE TARTRATE:Hot flush:0.13), (ZIPRASIDONE HYDROCHLORIDEZIPRASIDONE MESYLATE:Hot flush:0.09), (ALCOHOL:Huntington's disease:0.2),(ARMODAFINIL:Huntington's disease:0.36), (BUDESONIDE:Huntington's disease:0.41), (BUPRENORPHINE HYDROCHLORIDE:Huntington's disease:0.08), (BUPRENORPHINE HYDROCHLORIDENALOXONE HYDROCHLORIDE:Huntington's disease:0.09), (CALCIUM CARBONATECHOLECALCIFEROL:Huntington's disease:0.32), (CERTOLIZUMAB PEGOL:Huntington's disease:0.5), (CLINDAMYCINCLINDAMYCIN PHOSPHATE:Huntington's disease:0.17), (DIPHENHYDRAMINE:Huntington's disease:0.21), (ELETRIPTAN HYDROBROMIDE:Huntington's disease:0.13), (ENALAPRIL:Huntington's disease:0.71), (ENOXAPARIN:Huntington's disease:0.09), (EPINEPHRINE:Huntington's disease:0.02), (EVOLOCUMAB:Huntington's disease:0.26), (FERROUS SULFATE:Huntington's disease:0.26), (FLUOXETINEFLUOXETINE HYDROCHLORIDE:Huntington's disease:0.16), (HYDROMORPHONE HYDROCHLORIDE:Huntington's disease:0.32), (IBRUTINIB:Huntington's disease:1.09), (LACOSAMIDE:Huntington's disease:0.01), (METAXALONE:Huntington's disease:0.4), (METHYLPHENIDATE HYDROCHLORIDE:Huntington's disease:0.15), (METHYLPREDNISOLONE SODIUM SUCCINATE:Huntington's disease:0.05), (MODAFINIL:Huntington's disease:0.13), (OXYGEN:Huntington's disease:0.22), (PRAMIPEXOLE DIHYDROCHLORIDE:Huntington's disease:0.52), (PROMETHAZINE HYDROCHLORIDE:Huntington's disease:0.01), (RABEPRAZOLE SODIUM:Huntington's disease:0.03), (RANOLAZINE:Huntington's disease:0.3), (SODIUM OXYBATE:Huntington's disease:0.45), (TERIFLUNOMIDE:Huntington's disease:0.6), (TRIAMCINOLONE ACETONIDE:Huntington's disease:0.04), (VARENICLINE TARTRATE:Huntington's disease:1.08), (ZONISAMIDE:Huntington's disease:0.04), (ETHAMBUTOL HYDROCHLORIDE:Hydrocephalus:0.73), (UNSPECIFIED INGREDIENT:Hyper IgM syndrome:0.33), (ACARBOSE:Hypercalcaemia:0.03), (ALCOHOL:Hypercalcaemia:0.14), (ALE NDRONIC ACID:Hypercalcaemia:0.36), (AMPHETAMINE ASPARTATEAMPHETAMINE SULFATEDEXTROAMPHETAMINE SACCHARATEDEXTROAMPHETAMINE SULFATE:Hypercalcaemia:0.17), (BUDESONIDE:Hypercalcaemia:1.04), (BUPROPION:Hypercalcaemia: 0.34), (CANAKINUMAB:Hypercalcaemia:0.11), (CANDESARTAN CILEXETILHYDROCHLOROTHIAZIDE:Hypercalcaemia:0.68), (CERTOLIZUMAB PEGOL:Hypercalcaemia:0.23), (CLOZAPINE:Hypercalcaemia:0.61), (COLCHICINE:Hypercalcaemia: 0.48), (CORTISONEHYDROCORTISONE:Hypercalcaemia:0.15), (CRIZOTINIB:Hypercalcaemia: 0.22), (CYCLOBENZAPRINE:Hypercalcaemia:0.23), (DALTEPARIN:Hypercalcaemia: 0), (DICYCLOMINE HYDROCHLORIDE:Hypercalcaemia:0.07), (DULOXETINE:Hypercalcaemia:0.04), (ELOTUZ UMAB:Hypercalcaemia:0.1), (EPIRUBICIN:Hypercalcaemia:0.41), (EPLERENONE:Hypercalcaemia: 0.01), (FERROUS SULFATE:Hypercalcaemia:0.87), (FERROUS SULFATEFERROUS SULFATE, DRIED:Hypercalcaemia:1.17), (FESOTERODINE FUMARATE:Hypercalcaemia:0.11), (GLYBURIDE:Hypercalcaemia:0.02), (HYDROCHLORO THIAZIDEIRBESARTAN:Hypercalcaemia:0.13), (HYDROCHLOROTHIAZIDEVALSARTA N:Hypercalcaemia:0.14), (IDELALISIB:Hypercalcaemia:0.03), (IMATINIB MESYLATE:Hypercalcaemia:1.23), (INDAPAMIDE:Hypercalcaemia:0.08), (INDAPAMIDEPE RINDOPRIL:Hypercalcaemia:0.28), (IPRATROPIUM:Hypercalcaemia:0.05), (ISOSORBIDE: Hypercalcaemia:0.3), (ISOSORBIDE MONONITRATE:Hypercalcaemia:0.92), (LERCANIDIPINE HYDROCHLORIDE:Hypercalcaemia:0.42), (LITHIUM CARBONATE:Hypercalcaemia:0.29), (MESNA:Hypercalcaemia:0.04), (METAXALONE:Hypercalcaemia: 0.08), (MOXIFLOXACIN:Hypercalcaemia:0.1), (NADROPARIN CALCIUM:Hypercalcaemia:0.13), (OLAPARIB:Hypercalcaemia:0.02), (OMEGA-3-ACID ETHYL ESTERS:Hypercalcaemia:0.19), (OXCARBAZEPINE:Hypercalcaemia:0.4), (PAZOPANIB HYDROCHLORIDE:Hypercalcaemia:0.57), (PEMETREXED:Hypercalcaemia:0.14), (PENICIL LIN:Hypercalcaemia:0.07), (PERTUZUMAB:Hypercalcaemia:0.25), (PONATINIB:Hypercalcaemia: 0.64), (PRIMIDONE:Hypercalcaemia:0.23), (RANOLAZINE:Hypercalcaemia:0.44), (SACU BITRILVALSARTAN:Hypercalcaemia:0.53), (SECUKINUMAB:Hypercalcaemia:0.12), (TIMO LOL:Hypercalcaemia:0.04), (TOPIRAMATE:Hypercalcaemia:0.77), (TORSEMIDE:Hypercalcaemia: 1.55), (VANCOMYCIN HYDROCHLORIDE:Hypercalcaemia:0.34), (VINCRISTINE SULFATE:Hypercalcaemia:0.63), (.ALPHA.1-PROTEINASE INHIBITOR HUMAN:Hyperchlorhydria:0.34), (ACETAMINOPHENOXYCODONE HYDROCHLORIDEOXYCODONE TEREPHTHALATE:Hyperchlorhydria:0.02), (ALBIGLUTIDE:Hyperchlorhydria:0.08), (AMIO DARONE HYDROCHLORIDE:Hyperchlorhydria:1.14), (ATROPINE SULFATEDIPHENOXYLATE HYDROCHLORIDE:Hyperchlorhydria:0.15), (BENAZEPRILBENAZEPRIL HYDROCHLORIDE:Hyperchlorhydria:0.42), (BICALUTAMIDE:Hyperchlorhydria:0.61), (BIS ACODYL OR DOCUSATE SODIUM:Hyperchlorhydria:0.98), (BROMAZEPAM:Hyperchlorhydria:0.3), (BUDESONIDEF ORMOTEROL FUMARATE:Hyperchlorhydria:0.41), (BUPRENORPHINE HYDROCHLORIDE:Hyperchlorhydria:0.07), (BUPRENORPHINE HYDROCHLORIDENALOXONE HYDROCHLORIDE:Hyperchlorhydria:0.15), (CANAGLIFLOZINMETFORMIN HYDROCHLORIDE:Hyperchlorhydria:0.27), (CANAKINUMAB:Hyperchlorhydria:0.2), (CAPT OPRIL:Hyperchlorhydria:0.45), (CARFILZOMIB:Hyperchlorhydria:0.81), (CARVEDILOL PHOSPHATE:Hyperchlorhydria:0.8), (CHLORTHALIDONE:Hyperchlorhydria:0.34), (CILAST ATIN SODIUMIMIPENEM:Hyperchlorhydria:0.13), (DABIGATRAN:Hyperchlorhydria:0.03), (DAC LIZUMAB:Hyperchlorhydria:0.7), (DALTEPARIN SODIUM:Hyperchlorhydria:0.06), (DANAZOL:Hyperchlorhydria:0.09), (DAPAGLIFLOZIN PROPANEDIOL:Hyperchlorhydria:0.44), (DASATINIB:Hyperchlorhydria:1.04), (DEFLAZACO RT:Hyperchlorhydria:0.03), (DEXAMETHASONE ACETATE:Hyperchlorhydria:0.04), (DOXEPIN HYDROCHLORIDE:Hyperchlorhydria:0.19), (DRONABINOL:Hyperchlorhydria:0.5), (EDOXA BAN TOSYLATE:Hyperchlorhydria:0.84), (EMPAGLIFLOZINLINAGLIPTIN:Hyperchlorhydria:0.28), (EPINEPHRINE:Hyperchlorhydria:1.07), (ESTROGENS, CONJUGATEDMEDROXYPROGESTERONE ACETATE:Hyperchlorhydria:0.18), (EVOLOCUMAB:Hyperchlorhydria:1.3), (FLUOXETINEF LUOXETINE HYDROCHLORIDE:Hyperchlorhydria:0.64), (FLUTICASONE FUROATESALMETEROL XINAFOATE:Hyperchlorhydria:0.41), (FORMOTEROL FUMARATE:Hyperchlorhydria:0.39), (FULVESTRANT:Hyperchlorhydria:0.54), (GLYCOPYR RONIUMINDACATEROL:Hyperchlorhydria:0.49), (HYDROXYUREA:Hyperchlorhydria:0.2), (ILOPROST:Hyperchlorhydria:0.82), (IMIPRAMINE HYDROCHLORIDE:Hyperchlorhydria:0.45), (LAPATINIB DITOSYLATE:Hyperchlorhydria:0.03), (LEFLUNOMIDE:Hyperchlorhydria:0.25), (LERCANI DIPINE:Hyperchlorhydria:0.72), (LERCANIDIPINE HYDROCHLORIDE:Hyperchlorhydria:0.38), (LIDOCAINE:Hyperchlorhydria:1.15), (LITHIUM CARBONATE:Hyperchlorhydria:0.7), (LOMITAPIDE MESYLATE:Hyperchlorhydria:0.19), (LOPERAMIDE:Hyperchlorhydria:0.06), (MEPOLIZUM AB:Hyperchlorhydria:0.11), (METHENAMINE HIPPURATE:Hyperchlorhydria:0.22), (METHYLPREDNISOLONE ACETATE:Hyperchlorhydria:0.35), (NATEGLINIDE:Hyperchlorhydria:0.23), (NILOTINIB: Hyperchlorhydria:1.3), (NINTEDANIB:Hyperchlorhydria:0.5), (NITROFURANTOIN:Hyperchlorhydria: 1.05), (OMEGA-3-ACID ETHYL ESTERS:Hyperchlorhydria:1.13), (PAROXETINEPAROXETINE HYDROCHLORIDE:Hyperchlorhydria:1.09), (PAZOPANIB HYDROCHLORIDE:Hyperchlorhydria:0.78), (PEGINTERFERON BETA-1A:Hyperchlorhydria:0.52), (PEMETREXED DISODIUM:Hyperchlorhydria:0.05), (PHENOBARBITAL:Hyperchlorhydria:0.2), (PHENYTOI N:Hyperchlorhydria:0.1), (PHYTONADIONE:Hyperchlorhydria:0.16), (POLYETHYLENE GLYCOLS:Hyperchlorhydria:1.42), (PONATINIB:Hyperchlorhydria:0.16), (PRAZOSIN:Hyperchlorhydria: 0.01), (PREDNISOLONEPREDNISOLONE ACETATEPREDNISOLONE SODIUM SUCCINATE:Hyperchlorhydria:0.57), (PROGESTERONE:Hyperchlorhydria:0.41), (RALOXIF ENE HYDROCHLORIDE:Hyperchlorhydria:0.11), (RALTEGRAVIR POTASSIUM:Hyperchlorhydria:0.1), (ROFLUMILAST:Hyperchlorhydria:0.78), (SACUBITRIL VALSARTAN:Hyperchlorhydria:1.22), (SITAGLIPTIN:Hyperchlorhydria:0.01), (SULFAMET HOXAZOLE:Hyperchlorhydria:0.26), (SUMATRIPTAN:Hyperchlorhydria:0.2), (TETRACYCLI NE:Hyperchlorhydria:0.29), (THALIDOMIDE:Hyperchlorhydria:0.67), (TIMOLOL:Hyperchlorhydria: 0.02), (TOCOPHEROL:Hyperchlorhydria:0), (UMECLIDINIUM BROMIDEVILANTEROL TRIFENATATE:Hyperchlorhydria:1.04), (VALGANCICLOVIR HYDROCHLORIDE:Hyperchlorhydria:0.11), (VITAMIN A:Hyperchlorhydria:0.28), (.ALPHA.-TOCOPHEROLRETINOL:Hypercholesterolaemia:0.36), (.BETA.-CAROTENE:Hypercholesterolaemia:0), (ABOBOTULINUMTOXINA:Hypercholesterolaemia:1.12), (ACAMPROSATE:Hypercholesterolaemia:0.59), (ACETAMINOPHENAPRONALIDEC AFFEINEPROPYPHENAZONE:Hypercholesterolaemia:0.02), (ACETAMINOPHENMETHO CARBAMOL:Hypercholesterolaemia:0.43), (ACETYLCARNITINE HYDROCHLORIDE:Hypercholesterolaemia:0.58), (ACLIDINIUM BROMIDEFORMOTEROL FUMARATE:Hypercholesterolaemia:0.34), (ADENINE:Hypercholesterolaemia:0.74), (AJMALI NE:Hypercholesterolaemia:0.34), (ALBUMIN BOVINETANNIC ACID:Hypercholesterolaemia:0.3), (ALBUTEROLGUAIFENESIN:Hypercholesterolaemia:0.09), (ALDESLEUKIN:Hypercholesterolaemia:1.03), (ALGLUCOSIDASE ALFA:Hypercholesterolaemia:0.22), (ALISKIREN HEMIFUMARATEHYDROCHLOROTHIAZIDE:Hypercholesterolaemia:0.22), (ALUMINUM HYDROXIDEMAGNESIUM HYDROXIDESIMETHICONE:Hypercholesterolaemia:0.25), (ALUMINUM HYDROXIDEMAGNESIUM OXIDEOXETHAZAINE:Hypercholesterolaemia:0.61), (AMINO ACIDSDEXTROSEELECTROLYTES NOS:Hypercholesterolaemia:1.21), (AMINO ACIDSDEXTROSEELECTROLYTES NOSSOYBEAN OIL:Hypercholesterolaemia:0.16), (AMINOSALICYLIC ACID:Hypercholesterolaemia:0.17), (AMMONIUM CHLORIDEDEXTROMETHORPHANSODIUM CITRATE:Hypercholesterolaemia:0.55), (AMMONIUM LACTATE:Hypercholesterolaemia:1.34), (AMOXICILLINCLARITHROMYCINESOMEPRA ZOLE:Hypercholesterolaemia:1.36), (AMPICILLIN SODIUM:Hypercholesterolaemia:0.2), (ANAKINRA:Hypercholesterolaemia:0.47), (ARIPIPRAZ OLE LAUROXIL:Hypercholesterolaemia:0.79), (ASCORBIC ACID/CHROMIUM/SELENIUM/VANADYL SULFATE:Hypercholesterolaemia:0), (ASCORBIC ACIDBIOTINCALCIUM PANTOTHENATECYANOCOBALAMINFOLIC ACIDNIACINAMIDEPYRIDOXINE HYDROCHLORIDERIBOFLAVINTHIAMINE MONONITRATE:Hypercholesterolaemia:0.18), (ASCORBIC ACIDBIOTINCYANOCOBALAMINFOLIC ACIDNIACINPANTOTHENIC ACIDPYRIDOXINERIBOFLAVINTHIAMINE:Hypercholesterolaemia:0.54), (ASCORBIC ACIDFERROUS SULFATE:Hypercholesterolaemia:0.56), (ASCORBIC ACIDFERROUS SULFATEFOLIC ACIDPYRIDOXINE HYDROCHLORIDERIBOFLAVINTHIAMINE MONONITRATE:Hypercholesterolaemia:0.06), (ASCORBIC ACIDPOLYETHYLENE GLYCOL 3350\POTASSIUM CHLORIDESODIUM ASCORBATESODIUM CHLORIDESODIUM SULFATE:Hypercholesterolaemia:0.09), (ASIAN GINSENG:Hypercholesterolaemia:0.49), (BALSALAZIDE:Hypercholesterolaemia:0.25), (BAMI FYLLINE HYDROCHLORIDE:Hypercholesterolaemia:0.84), (BENIDIPINE:Hypercholesterolaemia:1.08), (BENZOCAINE:Hypercholesterolaemia:0.41), (BETHANECHOL CHLORIDE:Hypercholesterolaemia:1.47), (BIPERIDEN:Hypercholesterolaemia:0.45), (BLINAT UMOMAB:Hypercholesterolaemia:1.02), (BUFORMIN:Hypercholesterolaemia:0.11), (BUPIVA CAINE HYDROCHLORIDE:Hypercholesterolaemia:0.51), (CABOZANTINIB:Hypercholesterolaemia:00.71), (CALCIUM BICARBONATE:Hypercholesterolaemia:0.16), (CALCIUM LACTATE:Hypercholesterolaemia:1.37), (CALCIUM PANTOTHENATECYANOCOBALAMINNIACINAMIDEPYRIDOXINE HYDROCHLORIDERIBOFLAVINTHIAMINE MONONITRATE:Hypercholesterolaemia:0.95), (CALCIUMMAGNESIUMZINC:Hypercholesterolaemia: 1.26), (CANNABIDIOL:Hypercholesterolaemia:0.26), (CANRENOIC ACID:Hypercholesterolaemia:0.1), (CARBIDOPA:Hypercholesterolaemia:1.65), (CARPERITID E:Hypercholesterolaemia:0.11), (CEFOTAXIMECEFOTAXIME SODIUM:Hypercholesterolaemia:1.35), (CEFPODOXIME PROXETIL:Hypercholesterolaemia:1.49), (CEFTAZIDIME SODIUM:Hypercholesterolaemia:0.26), (CELLULASEDIASTASELIPASE:Hypercholesterolaemia: 0.26), (CERIVASTATIN SODIUM:Hypercholesterolaemia:0.2), (CETIRIZINE HYDROCHLORIDEPSEUDOEPHEDRINE HYDROCHLORIDE:Hypercholesterolaemia:0.79), (CHIMAPHILA UMBELLATAMANGANESE CHLORIDEPOPULUS TREMULOIDESSODIUM TAUROCHOLATE:Hypercholesterolaemia:0.2), (CHOLESTEROL:Hypercholesterolaemia:0.23), (CICLOPIROX OLAMINE:Hypercholesterolaemia:0.35), (CIFENLINE SUCCINATE:Hypercholesterolaemia:0.34), (CLADRIBINE:Hypercholesterolaemia:0.05), (CLE NBUTEROL HYDROCHLORIDE:Hypercholesterolaemia:0.58), (CLIKSTAR:Hypercholesterolaemia:1.03), (CLOBETASONE BUTYRATE:Hypercholesterolaemia:0.19), (COLCHICINEOPIUMTIEMONIUM METHYLSULFATE:Hypercholesterolaemia:0.21), (CORTICOTROPIN:Hypercholesterolaemia: 1.64), (CRANBERRY JUICE:Hypercholesterolaemia:0.38), (CROTAMITON:Hypercholesterolaemia:0.14), (CYANOC OBALAMINFOLIC ACIDPYRIDOXINE:Hypercholesterolaemia:0.32), (DARUNAVIRRITONAVIR:Hypercholesterolaemia: 0.37), (DEMECLOCYCLINE:Hypercholesterolaemia:0.01), (DESFLURANE:Hypercholesterolaemia: 0.15), (DEXAMETHASONEOXYTETRACYCLINE:Hypercholesterolaemia:0.16), (DEXPANTHENOLNIACINAMIDEPYRIDOXINE HYDROCHLORIDERIBOFLAVIN 5′-PHOSPHATE SODIUMTHIAMINE HYDROCHLORIDE:Hypercholesterolaemia:0.1), (DIAZOXIDE:Hypercholesterolaemia:0.24), (DIFLUNISAL:Hypercholesterolaemia:0.47), (DIGITALIS:Hypercholesterolaemia:0.6), (DIHYD ROCODEINE PHOSPHATE:Hypercholesterolaemia:0.03), (DIPYRONEPROMETHAZINE HYDROCHLORIDE:Hypercholesterolaemia:0.51), (DISOPYRAMIDE:Hypercholesterolaemia: 0.79), (DOBUTAMINE HYDROCHLORIDE:Hypercholesterolaemia:0.99), (DOLASETRON MESYLATE:Hypercholesterolaemia:1.09), (DOPAMINEDOPAMINE HYDROCHLORIDE:Hypercholesterolaemia:0.7), (DOXYLAMINE SUCCINATEPYRIDOXINE HYDROCHLORIDE:Hypercholesterolaemia:0.41), (ECABET SODIUM:Hypercholesterolaemia:0.38), (ECONAZOLE:Hypercholesterolaemia:3.61), (ELBASV IRGRAZOPREVIR:Hypercholesterolaemia:0.8), (ELTROMBOPAG OLAMINE:Hypercholesterolaemia:0.37), (EMPAGLIFLOZINMETFORMIN HYDROCHLORIDE:Hypercholesterolaemia:0.59), (EMTRICITABINERILPIVIRINE HYDROCHLORIDETENOFOVIR DISOPROXIL FUMARATE:Hypercholesterolaemia:0.61), (EPACADOSTAT:Hypercholesterolaemia:0.16), (EP ERISONE HYDROCHLORIDE:Hypercholesterolaemia:1.07), (ERGOLOID MESYLATES:Hypercholesterolaemia:0.14), (ERIBULIN MESYLATE:Hypercholesterolaemia:0.65), (ETHACRYNIC ACID:Hypercholesterolaemia:0.73), (ETHINYL ESTRADIOLNORETHINDRONE:Hypercholesterolaemia:0.49), (ETIFOXINE:Hypercholesterolaemia: 0.18), (FERRIC HYDROXIDE:Hypercholesterolaemia:1.33), (FERROUS BISGLYCINATE:Hypercholesterolaemia:0.9), (FLUNARIZINE HYDROCHLORIDE:Hypercholesterolaemia:0.74), (FLUOROURACILLEUCOVORIN CALCIUMOXALIPLATIN:Hypercholesterolaemia:0.32), (FLUOXETINE HYDROCHLORIDEOLANZAPINE:Hypercholesterolaemia:0.08), (FORMOTEROL FUMARATEMOMETASONE FUROATE:Hypercholesterolaemia:1.69), (FOSPHENYTOINFOSPHENYTOIN SODIUM:Hypercholesterolaemia:0.5), (FURSULTIAMINE:Hypercholesterolaemia:1.02), (FUSI DATE SODIUM:Hypercholesterolaemia:0.08), (GADOPENTETATE DIMEGLUMINE:Hypercholesterolaemia:0.38), (GADOXETATE DISODIUM:Hypercholesterolaemia:0.34), (GIMERACILOTERACILTEGAFUR:Hypercholesterolaemia: 1.39), (GLIMEPIRIDEROSIGLITAZONE MALEATE:Hypercholesterolaemia:0.81), (GLYCOPYRROLATEINDACATEROL MALEATE:Hypercholesterolaemia:1.38), (GUANFACINE HYDROCHLORIDE:Hypercholesterolaemia:0.93), (HERBALSKARAYA GUM:Hypercholesterolaemia:0.15), (HYDROCODONE BITARTRATEIBUPROFEN:Hypercholesterolaemia:0.96), (HYDROCORTISONE ACETATE:Hypercholesterolaemia:2.41), (HYDROCORTISONE HEMISUCCINATE:Hypercholesterolaemia:0.02), (HYOSCYAMINE SULFATEMETHENAMINEMETHYLENE BLUEPHENYL SALICYLATESODIUM PHOSPHATE, MONOBASIC, MONOHYDRATE:Hypercholesterolaemia:0.12), (IBRITUMOMAB TIUXETAN:Hypercholesterolaemia:0.73), (IMIPENEM:Hypercholesterolaemia:0.73), (IMMUN E GLOBULIN NOS:Hypercholesterolaemia:0.11), (INTERFERON:Hypercholesterolaemia:2.82), (INTERFERO N ALFA-2A OR INTERFERON ALFA-2B:Hypercholesterolaemia:0.4), (INTERFERON BETA:Hypercholesterolaemia:0.02), (IPRAGLIFLOZIN:Hypercholesterolaemia:0.11), (IRONP OLYSACCHARIDES:Hypercholesterolaemia:0.45), (KETAMINE HYDROCHLORIDE:Hypercholesterolaemia:0), (LAPATINIB:Hypercholesterolaemia:0.21), (LE VOMILNACIPRAN HYDROCHLORIDE:Hypercholesterolaemia:0.28), (LEVOTHYROXINELIOTHYRONINE: Hypercholesterolaemia:0.47), (MAGNESIUM PIDOLATE:Hypercholesterolaemia:0.62), (MEDROXYPROGESTERONE:Hypercholesterolaemia: 0.96), (METFORMIN HYDROCHLORIDEROSIGLITAZONE MALEATE:Hypercholesterolaemia:1.05), (METHENAMINE MANDELATE:Hypercholesterolaemia:0.02), (METHENOLONE ENANTHATE:Hypercholesterolaemia:1.36), (MIGLUSTAT:Hypercholesterolaemia:0.12), (MIL RINONE LACTATE:Hypercholesterolaemia:0.08), (MINERALS:Hypercholesterolaemia:0.17), (MITOXA NTRONE HYDROCHLORIDE:Hypercholesterolaemia:0.51), (MOEXIPRIL HYDROCHLORIDE:Hypercholesterolaemia:1.69), (MORPHINE SULFATENALTREXONE HYDROCHLORIDE:Hypercholesterolaemia:1.67), (NALBUPHINE HYDROCHLORIDE:Hypercholesterolaemia:0.1), (NALFURAFINE HYDROCHLORIDE:Hypercholesterolaemia:0.17), (NALOXONE:Hypercholesterolaemia:0.62), (NARATRIPTAN HYDROCHLORIDE:Hypercholesterolaemia:0.66), (NEOMYCIN:Hypercholesterolaemia:0.53), (OBETICHOLIC ACID:Hypercholesterolaemia:0.16), (OLODATEROL HYDROCHLORIDE:Hypercholesterolaemia:0.29), (ORPHENADRINE:Hypercholesterolaemia: 1.54), (OXAPROZIN:Hypercholesterolaemia:0.64), (OXETORONE:Hypercholesterolaemia:0.11), (OXYBUTYNINOXYBUTYNIN CHLORIDE:Hypercholesterolaemia:0.99), (PENICILLAMINE:Hypercholesterolaemia:0.31), (PE NICILLIN V BENZATHINE:Hypercholesterolaemia:0.45), (PENTAMIDINE:Hypercholesterolaemia:0.3), (PE ROSPIRONE:Hypercholesterolaemia:0.01), (PEROSPIRONE HYDROCHLORIDE:Hypercholesterolaemia:0.22), (PINAVERIUM BROMIDE:Hypercholesterolaemia:0.16), (PIROXICAM BETADEX:Hypercholesterolaemia:0.12), (PIZOTYLINE:Hypercholesterolaemia:0.74), (POLYE THYLENE GLYCOL 3350\POTASSIUM CHLORIDESODIUM BICARBONATESODIUM CHLORIDESODIUM SULFATE ANHYDROUS:Hypercholesterolaemia:1.3), (POLYSTYRENE SULFONIC ACID:Hypercholesterolaemia:0.42), (PORFIMER SODIUM:Hypercholesterolaemia:0.18), (POTASSIUM CITRATETRISODIUM CITRATE DIHYDRATE:Hypercholesterolaemia:0.87), (POTASSIUM IODIDE:Hypercholesterolaemia:0.58), (PROCHLORPERAZINE EDISYLATE:Hypercholesterolaemia:0.21), (PROPARACAINE:Hypercholesterolaemia:0.05), (P ROTRIPTYLINE HYDROCHLORIDE:Hypercholesterolaemia:0.43), (PYRIDOXAL:Hypercholesterolaemia:0.57), (PYRIDOXAL PHOSPHATE:Hypercholesterolaemia:0.57), (PYRIMETHAMINE:Hypercholesterolaemia:0.43), (QUINACRINE:Hypercholesterolaemia:1.27), (RAMUCIRUMAB:Hypercholesterolaemia:1.31), (RILPIVIRINE:Hypercholesterolaemia:0.09), (RILPIVIRINE HYDROCHLORIDE:Hypercholesterolaemia:1.44), (RUCAPARIB CAMSYLATE:Hypercholesterolaemia:0.19), (RUPATADINE FUMARATE:Hypercholesterolaemia:0.25), (SARPOGRELATE HYDROCHLORIDE:Hypercholesterolaemia:0.63), (SERRAPEPTASE:Hypercholesterolaemia:00.49), (SIBUTRAMINE HYDROCHLORIDE:Hypercholesterolaemia:0.56), (SILICON DIOXIDE:Hypercholesterolaemia:0.19), (SITAFLOXACIN:Hypercholesterolaemia:0.05), (SODI UM CITRATE:Hypercholesterolaemia:0.35), (SODIUM FLUORIDE:Hypercholesterolaemia:1.72), (SODIUM PHOSPHATE, DIBASIC, ANHYDROUSSODIUM PHOSPHATE, DIBASIC, HEPTAHYDRATESODIUM PHOSPHATE, MONOBASIC, MONOHYDRATE:Hypercholesterolaemia:0.02), (SOFOSBUVIRVELPATASVIR:Hypercholesterolaemia: 1.08), (SPIRULINA:Hypercholesterolaemia:0.05), (SULTOPRIDE HYDROCHLORIDE:Hypercholesterolaemia:0.11), (SUNITINIB:Hypercholesterolaemia:0.08), (TEDUGLUTIDE:Hypercholesterolaemia:0.17), (TEDUGLUTIDEWATER:Hypercholesterolaemia: 0.81), (TELOTRISTAT ETHYL:Hypercholesterolaemia:0.78), (THEOPHYLLINE:Hypercholesterolaemia:0.76), (THIOP ENTAL SODIUM:Hypercholesterolaemia:0.03), (THYROID, PORCINETHYROID, UNSPECIFIED:Hypercholesterolaemia:0.47), (TIANEPTINE SODIUM:Hypercholesterolaemia:0.05), (TILIDINE:Hypercholesterolaemia:0.98), (TOREMIFEN E CITRATE:Hypercholesterolaemia:0.14), (TROSPIUM:Hypercholesterolaemia:0.8), (UROKINAS E:Hypercholesterolaemia:0.51), (VALDECOXIB:Hypercholesterolaemia:0.64), (VANDETANIB: Hypercholesterolaemia:0.38), (VINORELBINEVINORELBINE TARTRATE:Hypercholesterolaemia:0.59), (VONOPRAZAN:Hypercholesterolaemia:0.06), (VO RAPAXAR SULFATE:Hypercholesterolaemia:0.14), (YEAST:Hypercholesterolaemia:1.06), (ZAFIRLUKAS T:Hypercholesterolaemia:1.48), (ZALTOPROFEN:Hypercholesterolaemia:0.01), (ZIPRASIDON E HYDROCHLORIDE:Hypercholesterolaemia:0.07), (ZIV-AFLIBERCEPT:Hypercholesterolaemia:0.5), (ENALAPRIL:Hypercoagulation:0.21), (EPINEPH RINE:Hypercoagulation:0.11), (FERROUS SULFATEFERROUS SULFATE, DRIED:Hypercoagulation:0.09), (FISH OIL:Hypercoagulation:0.05), (NAPROXEN:Hypercoagulation:0.31), (RAMIPRIL:Hypercoagulation: 0.2), (ACEBUTOLOL HYDROCHLORIDE:Hyperglycaemia:0), (ACLIDINIUM BROMIDE:Hyperglycaemia:0.03), (AFATINIB:Hyperglycaemia:0.04), (AGALSIDASE BETA:Hyperglycaemia:0.77), (ALDESLEUKIN:Hyperglycaemia:0.14), (ALUMINUM HYDROXIDEDIMETHICONEMAGNESIUM HYDROXIDE:Hyperglycaemia:0.5), (ALUMINUM HYDROXIDEMAGNESIUM OXIDEOXETHAZAINE:Hyperglycaemia:0), (AMINO ACIDSDEXTROSEELECTROLYTES NOS:Hyperglycaemia:0.26), (AMISULPRIDE:Hyperglycaemia:0.28), (AMITRIPTYLINE HYDROCHLORIDE:Hyperglycaemia:0.58), (AMLODIPINE BESYLATEHYDROCHLOROTHIAZIDEVALSARTAN:Hyperglycaemia:0.01), (ATRACUR IUM:Hyperglycaemia:0.2), (AXITINIB:Hyperglycaemia:0.43), (BELATACEPT:Hyperglycaemia: 0.38), (BERAPROST SODIUM:Hyperglycaemia:0.24), (BIFIDOBACTERIUM SPP.:Hyperglycaemia:0.14), (BILASTINE:Hyperglycaemia:0.03), (BIMATOPROST:Hyperglycaemia: 0.06), (BUPRENORPHINE HYDROCHLORIDE:Hyperglycaemia:0.68), (BUPROPION:Hyperglycaemia:0.89), (CALCITO NIN SALMON:Hyperglycaemia:0.37), (CANAKINUMAB:Hyperglycaemia:0.18), (CARVEDILOL PHOSPHATE:Hyperglycaemia:0.93), (CEFOTAXIMECEFOTAXIME SODIUM:Hyperglycaemia:0.13), (CERTOLIZUMAB PEGOL:Hyperglycaemia:1.35), (CINACALCET HYDROCHLORIDE:Hyperglycaemia:0.06), (CISATRACURIUM:Hyperglycaemia:0.4), (CLIN DAMYCIN:Hyperglycaemia:1.24), (DABIGATRAN:Hyperglycaemia:0.94), (DABRAFENIB MESYLATE:Hyperglycaemia:0.1), (DACLATASVIR:Hyperglycaemia:0.08), (DACLIZUMAB: Hyperglycaemia:1.15), (DALFAMPRIDINE:Hyperglycaemia:0.22), (DANAZOL:Hyperglycaemia: 0.85), (DESFLURANE:Hyperglycaemia:0.45), (DESLORATADINE:Hyperglycaemia:0.88), (DI VALPROEX SODIUM:Hyperglycaemia:0.8), (DOFETILIDE:Hyperglycaemia:0.71), (DOLASETRON MESYLATE:Hyperglycaemia:0.21), (DOXEPIN:Hyperglycaemia:0.27), (DRONEDARONE: Hyperglycaemia:0.13), (EDOXABAN TOSYLATE:Hyperglycaemia:1.39), (ELETRIPTAN HYDROBROMIDE:Hyperglycaemia:0.46), (EMPAGLIFLOZINMETFORMIN HYDROCHLORIDE:Hyperglycaemia:0.13), (EPINASTINE HYDROCHLORIDE:Hyperglycaemia:0.17), (EPIRUBICIN:Hyperglycaemia:0.99), (ESMOLOL HYDROCHLORIDE:Hyperglycaemia:0.66), (ESTROGENS, CONJUGATED:Hyperglycaemia:0.6), (ESZOPICLONE:Hyperglycaemia:0.96), (ETORICOXIB: Hyperglycaemia:0.38), (FENOFIBRIC ACID:Hyperglycaemia:0.15), (FERROUS FUMARATE:Hyperglycaemia:0.47), (FERROUS GLUCONATE:Hyperglycaemia:0.09), (FESOTERODINE FUMARATE:Hyperglycaemia:0.97), (FLECAINIDE:Hyperglycaemia:1.41), (FLUINDIONE: Hyperglycaemia:0.71), (FLUOXETINEFLUOXETINE HYDROCHLORIDE:Hyperglycaemia:0.73), (FLUTICASONE:Hyperglycaemia:0.9), (FLUTICA SONE FUROATE:Hyperglycaemia:0.15), (FLUTICASONE FUROATESALMETEROL XINAFOATE:Hyperglycaemia:0.84), (FLUTICASONE FUROATEVILANTEROL TRIFENATATE:Hyperglycaemia:0.81), (FONDAPARINUX SODIUM:Hyperglycaemia:0.26), (FORMOTEROL FUMARATE:Hyperglycaemia:0.47), (FORMOTEROL FUMARATE DIHYDRATEMOMETASONE FUROATE:Hyperglycaemia:0.32), (FOSFOMYCIN:Hyperglycaemia:0.19), (FULVESTRANT: Hyperglycaemia:0.91), (GADOLINIUM:Hyperglycaemia:0.47), (GEFITINIB:Hyperglycaemia:0.25), (GIMERACILOTERACILTEGAFUR:Hyperglycaemia:0.72), (GLYCOPYRRONIUMINDA CATEROL:Hyperglycaemia:1.07), (HERBALSTURMERIC:Hyperglycaemia:0.11), (HYOSCY AMINE:Hyperglycaemia:0.01), (IBRITUMOMAB TIUXETAN:Hyperglycaemia:0.27), (ILOPROST:Hyperglycaemia:0.65), (IMIPRAMINE HYDROCHLORIDE:Hyperglycaemia:0.2), (IMMUNE GLOBULIN NOS:Hyperglycaemia:0.15), (INTERFERON:Hyperglycaemia:0.34), (INTERFERON ALFA-2A:Hyperglycaemia:0.52), (INTERFERON ALFA-2B:Hyperglycaemia:0.66), (LANREOTIDE ACETATE:Hyperglycaemia:0.11), (LAPATINIB DITOSYLATE:Hyperglycaemia:0.4), (LEFLUNOMIDE:Hyperglycaemia:0.16), (LERCANIDIPI NE:Hyperglycaemia:2.78), (LERCANIDIPINE HYDROCHLORIDE:Hyperglycaemia:1.38), (LEVALBUTEROL HYDROCHLORIDE:Hyperglycaemia:0.93), (LEVOLEUCOVORIN CALCIUM:Hyperglycaemia:0.42), (LITHIUM CARBONATE:Hyperglycaemia:1.28), (MAGNESIUM ASPARTATE HYDROCHLORIDE:Hyperglycaemia:0.03), (MEPERIDINE HYDROCHLORIDE:Hyperglycaemia:0.13), (METAXALONE:Hyperglycaemia:0.6), (METHEN OLONE ENANTHATE:Hyperglycaemia:0.2), (METHYLPREDNISOLONE ACETATE:Hyperglycaemia:0.52), (MIRABEGRON:Hyperglycaemia:0.29), (MOXONIDINE: Hyperglycaemia:0.77), (NABUMETONE:Hyperglycaemia:0.13), (NADOLOL:Hyperglycaemia:0.57), (NALOXONE HYDROCHLORIDETILIDINE HYDROCHLORIDE:Hyperglycaemia:0.49), (NEOSTIGMINE:Hyperglycaemia:0.58), (NINTED ANIB:Hyperglycaemia:0.5), (NITROFURANTOINNITROFURANTOIN MONOHYDRATE:Hyperglycaemia:0.1), (NOREPINEPHRINE:Hyperglycaemia:1.21), (NORTR IPTYLINE:Hyperglycaemia:0.5), (OLAPARIB:Hyperglycaemia:0.25), (OLODATEROL HYDROCHLORIDETIOTROPIUM BROMIDE MONOHYDRATE:Hyperglycaemia:0.24), (OXCARBAZEPINE:Hyperglycaemia:1.24), (PANT OPRAZOLE MAGNESIUM:Hyperglycaemia:0.91), (PAZOPANIB HYDROCHLORIDE:Hyperglycaemia:1.44), (PEGINTERFERON BETA-1A:Hyperglycaemia:0.2), (PEMETREXED DISODIUM:Hyperglycaemia:0.81), (PENTOSTATIN:Hyperglycaemia:0.55), (PENTOXIFYLLINE: Hyperglycaemia:0.03), (PILSICAINIDE HYDROCHLORIDE:Hyperglycaemia:0.06), (PIMOBENDAN:Hyperglycaemia:0.17), (PIROXICAM: Hyperglycaemia:0.14), (PRAMIPEXOLE DIHYDROCHLORIDE:Hyperglycaemia:0.63), (PRIMIDONE:Hyperglycaemia:0.75), (PRISTIN AMYCIN:Hyperglycaemia:0.13), (PSYLLIUM HUSK:Hyperglycaemia:0.11), (PYRIDOSTIGMINE BROMIDE:Hyperglycaemia:0.02), (PYRIDOXINE:Hyperglycaemia:0.4), (RADIATION THERAPY:Hyperglycaemia:0.51), (RALOXIFENE HYDROCHLORIDE:Hyperglycaemia:0.33), (RAMUCIRUMAB:Hyperglycaemia:0.47), (REGO RAFENIB:Hyperglycaemia:1.01), (RIZATRIPTAN BENZOATE:Hyperglycaemia:0.19), (ROFLUMILAST:Hyperglycaemia:0.67), (ROPINIROLE: Hyperglycaemia:0.22), (ROPINIROLE HYDROCHLORIDE:Hyperglycaemia:0.32), (SACCHAROMYCES CEREVISIAE:Hyperglycaemia:0.06), (SACUBITRILVALSARTAN:Hyperglycaemia:0.93), (SE VELAMER CARBONATE:Hyperglycaemia:0.35), (SEVELAMER HYDROCHLORIDE:Hyperglycaemia:0.49), (SEVOFLURANE:Hyperglycaemia:0.58), (SIPULE UCEL-T:Hyperglycaemia:0.11), (SODIUM FERROUS CITRATE:Hyperglycaemia:0.09), (SODIUM FLUORIDE:Hyperglycaemia:0.07), (SOTALOL HYDROCHLORIDE:Hyperglycaemia:0.02), (SUFENTANIL:Hyperglycaemia:0.38), (SUMATRI PTAN:Hyperglycaemia:0.43), (SUMATRIPTANSUMATRIPTAN SUCCINATE:Hyperglycaemia:0.22), (TERAZOSIN HYDROCHLORIDE:Hyperglycaemia:0.35), (TERBUTALINE:Hyperglycaemia:0.49), (TETRA CYCLINE:Hyperglycaemia:0.54), (THIOPENTAL:Hyperglycaemia:0.18), (TOCOPHEROL: Hyperglycaemia:0.02), (TROSPIUM CHLORIDE:Hyperglycaemia:0.37), (UMECLIDINIUM BROMIDEVILANTEROL TRIFENATATE:Hyperglycaemia:0.59), (VINCRISTINE SULFATE:Hyperglycaemia:1.12), (VINORELBINE TARTRATE:Hyperglycaemia:0.53), (VITAMIN B:Hyperglycaemia:0.8), (ZINC:Hyperglycaemia:0.27), (ANASTROZOLE:Hyperhidrosis:0.37), (CERTOLIZUMAB PEGOL:Hyperhidrosis:0.34), (DILTIAZEM:Hyperhidrosis:0.06), (DILTIAZEM HYDROCHLORIDE:Hyperhidrosis:0.35), (DIPHENHYDRAMINE:Hyperhidrosis:0.07), (DIVA LPROEX SODIUM:Hyperhidrosis:0.12), (EVOLOCUMAB:Hyperhidrosis:0.17), (FERROUS SULFATEFERROUS SULFATE, DRIED:Hyperhidrosis:0.09), (LOVASTATIN:Hyperhidrosis:0.76), (METHYLPHENIDATE HYDROCHLORIDE:Hyperhidrosis:0.26), (RALOXIFENE HYDROCHLORIDE:Hyperhidrosis:0.09), (RISEDRONATE SODIUM:Hyperhidrosis:0.13), (SACUBITRILVALSARTAN:Hyperhidrosis:0.15), (VARENIC LINE TARTRATE:Hyperhidrosis:0.6), (.ALPHA.-TOCOPHEROL ACETATE, DL-ASCORBIC ACIDCYANOCOBALAMINFLUORIDE IONFOLIC ACIDNIACINPYRIDOXINERIBOFLAVINTHIAMINEVITAMIN AVITAMIN D:Hyperkalaemia:0.18), (.ALPHA.1-PROTEINASE INHIBITOR HUMAN:Hyperkalaemia:0.26), (ACENOCOUMAROL:Hyperkalaemia:1.1), (ACETAMINOPH ENCAFFEINE:Hyperkalaemia:0.41), (ACETAMINOPHENPROPOXYPHENE HYDROCHLORIDE:Hyperkalaemia:0.25), (ACITRETIN:Hyperkalaemia:0.26), (AGALSIDASE BETA:Hyperkalaemia:1.07), (AMANTADINE:Hyperkalaemia:0.82), (AMINO ACIDSDEXTROSEELECTROLYTES NOS:Hyperkalaemia:0.59), (AMISULPRIDE:Hyperkalaemia:0.79), (AMLODIPINE BESYLATEIRBESARTAN:Hyperkalaemia:0.08), (AMLODIPINEVALSARTAN:Hyperkalaemia: 0.82), (AMMONIUM CHLORIDEDEXTROMETHORPHANSODIUM CITRATE:Hyperkalaemia:0.11), (AMPICILLIN SODIUMSULBACTAM SODIUM:Hyperkalaemia:0.28), (ANAGLIPTIN:Hyperkalaemia:0.03), (ASPIRINCLOPIDOGREL: Hyperkalaemia:0.19), (ASPIRINDIHYDROXYALUMINUM AMINOACETATE ANHYDROUSMAGNESIUM CARBONATE:Hyperkalaemia:0.07), (ASPIRINMAGNESIUM OXIDE:Hyperkalaemia:0.28), (ASUNAPREVIR:Hyperkalaemia:0.55), (BECLOMETHASONE: Hyperkalaemia:0.28), (BECLOMETHASONEFORMOTEROL:Hyperkalaemia:0.19), (BENDR OFLUMETHIAZIDE:Hyperkalaemia:0.21), (BEPRIDIL:Hyperkalaemia:0.85), (BETAMETHAS ONE DIPROPIONATECALCIPOTRIENE:Hyperkalaemia:0.11), (BETAMETHASONECALCIPOT RIENE:Hyperkalaemia:0.2), (BOCOCIZUMAB:Hyperkalaemia:0.11), (BUDESONIDEFORMO TEROL FUMARATE:Hyperkalaemia:0.84), (BUPRENORPHINE HYDROCHLORIDENALOXONE HYDROCHLORIDE:Hyperkalaemia:1.08), (CABAZITAXEL:Hyperkalaemia:0.34), (CALCITO NIN:Hyperkalaemia:0.67), (CALCIUMMAGNESIUM:Hyperkalaemia:0.33), (CALCIUMMAG NESIUMZINC:Hyperkalaemia:0.31), (CANAGLIFLOZINMETFORMIN HYDROCHLORIDE:Hyperkalaemia:1.54), (CARBIMAZOLE:Hyperkalaemia:0.55), (CARBOM ER:Hyperkalaemia:0.15), (CEFEPIMECEFEPIME HYDROCHLORIDE:Hyperkalaemia:0.26), (CERITINIB:Hyperkalaemia:0.48), (CERTOLIZUM AB PEGOL:Hyperkalaemia:1.69), (CETIRIZINE HYDROCHLORIDEPSEUDOEPHEDRINE HYDROCHLORIDE:Hyperkalaemia:0.59), (CHLORAMBUCIL:Hyperkalaemia:0.28), (CHOND ROITIN SULFATE AGLUCOSAMINE:Hyperkalaemia:0.28), (CIFENLINE:Hyperkalaemia:0.06), (CLIKSTAR: Hyperkalaemia:0.07), (COLESTIPOL:Hyperkalaemia:0.02), (DABIGATRAN:Hyperkalaemia:1.33), (DABRAFENIB:Hyperkalaemia:0.32), (DACLIZUMAB:Hyperkalaemia:0.96), (DALTEPARIN: Hyperkalaemia:1.1), (DANAZOL:Hyperkalaemia:1.37), (DAPAGLIFLOZIN PROPANEDIOLMETFORMIN HYDROCHLORIDE:Hyperkalaemia:0.14), (DASATINIB:Hyperkalaemia:2.47), (DICLOFENA C SODIUMMISOPROSTOL:Hyperkalaemia:0.12), (DIGITOXIN:Hyperkalaemia:0.89), (DIPYRO NE:Hyperkalaemia:0.54), (DOBUTAMINE HYDROCHLORIDE:Hyperkalaemia:0.42), (DOLASETRON MESYLATE:Hyperkalaemia:0.47), (DOPAMINE HYDROCHLORIDE:Hyperkalaemia:0.03), (DROXIDOPA:Hyperkalaemia:0.13), (EDOXABAN TOSILATE MONOHYDRATE:Hyperkalaemia:0.14), (ELOTUZUMAB:Hyperkalaemia:1.03), (ELTROMBO PAG OLAMINE:Hyperkalaemia:0.8), (ENALAPRIL MALEATELERCANIDIPINE HYDROCHLORIDE:Hyperkalaemia:0.33), (EPINASTINE HYDROCHLORIDE:Hyperkalaemia:0.69), (EPIRUBICIN:Hyperkalaemia:0.65), (ERIBULIN: Hyperkalaemia:0.05), (ESOMEPRAZOLE MAGNESIUMNAPROXEN:Hyperkalaemia:0.09), (ESTROGENS, CONJUGATEDMEDROXYPROGESTERONE ACETATE:Hyperkalaemia:1.02), (ESTRONESUS SCROFA OVARY:Hyperkalaemia:0.3), (ETORICOXIB:Hyperkalaemia:0.57), (EXEMESTANE:Hyperkalaemia: 0.81), (FENOTEROL HYDROBROMIDE:Hyperkalaemia:0.02), (FERROUS SULFATEGLYCINE:Hyperkalaemia:0.29), (FLUOROMETHOLONE:Hyperkalaemia:0.19), (F LUVOXAMINE MALEATE:Hyperkalaemia:0.61), (FONDAPARINUX:Hyperkalaemia:0.09), (FORMOTEROL: Hyperkalaemia:0.68), (FORMOTEROL FUMARATEMOMETASONE FUROATE:Hyperkalaemia:0.14), (FULVESTRANT:Hyperkalaemia:0.19), (GLIMEPIRIDERO SIGLITAZONE MALEATE:Hyperkalaemia:0.03), (GLYCOPYRROLATE:Hyperkalaemia:0.69), (GLYCOPYRR OLATEINDACATEROL MALEATE:Hyperkalaemia:0.13), (GLYCOPYRRONIUMINDACATEROL:Hyperkalaemia:1.22), (HYDROCHLOROTHIAZIDELOSARTAN:Hyperkalaemia:0.15), (HYDROCHLOROTHIA ZIDESPIRONOLACTONE:Hyperkalaemia:0.09), (HYDROCORTISONE ACETATE:Hyperkalaemia:0.44), (ILOPROST:Hyperkalaemia:1.46), (IMIGLUCERASE:Hyperkalaemia: 0.24), (IMIPRAMINE:Hyperkalaemia:0.12), (INTERFERON:Hyperkalaemia:0.4), (INTE RFERON ALFA-2A:Hyperkalaemia:0.49), (INTERFERON ALFA-2B:Hyperkalaemia:0.67), (INTERFERON GAMMA-1B:Hyperkalaemia:0.1), (INVESTIGATIONAL PRODUCT:Hyperkalaemia:0.89), (IRINOTECAN:Hyperkalaemia:0.47), (IRONPOLYSACCHA RIDES:Hyperkalaemia:0.02), (IXAZOMIB:Hyperkalaemia:0.98), (LACTASE:Hyperkalaemia:0.75), (LENVATINIB:Hyperkalaemia:0.23), (LERCANIDIPINE HYDROCHLORIDE:Hyperkalaemia:1.66), (LEVALBUTEROL:Hyperkalaemia:0.36), (LEVAL BUTEROL HYDROCHLORIDE:Hyperkalaemia:2.27), (LEVOLEUCOVORIN CALCIUM:Hyperkalaemia:0.82), (LITHIUM:Hyperkalaemia:0.5), (LOMITAPIDE MESYLATE:Hyperkalaemia:1.02), (MECLIZINEMECLIZINE HYDROCHLORIDE:Hyperkalaemia:1.05), (METFORMIN HYDROCHLORIDEROSIGLITAZONE MALEATE:Hyperkalaemia:0.31), (METFORMIN HYDROCHLORIDESAXAGLIPTIN:Hyperkalaemia:0.16), (METFORMIN HYDROCHLORIDEVILDAGLIPTIN:Hyperkalaemia:0.97), (METHENAMINE HIPPURATE:Hyperkalaemia:1.33), (METHYLPHENIDATE:Hyperkalaemia:0.2), (MIGLUSTA T:Hyperkalaemia:0.16), (MINODRONIC ACID:Hyperkalaemia:0.13), (MIZORIBINE:Hyperkalaemia:0.53), (MOEXIPRIL HYDROCHLORIDE:Hyperkalaemia:0.21), (MORPHINE SULFATENALTREXONE HYDROCHLORIDE:Hyperkalaemia:0.09), (NABUMETONE:Hyperkalaemia:1.09), (NADROP ARIN CALCIUM:Hyperkalaemia:0.9), (OLAPARIB:Hyperkalaemia:0.38), (OLMESARTAN:Hyperkalaemia: 0.47), (ONABOTULINUMTOXINA:Hyperkalaemia:0.13), (OSELTAMIVIR:Hyperkalaemia: 0.41), (OXYBUTYNINOXYBUTYNIN CHLORIDE:Hyperkalaemia:0.09), (PALBOCICLIB:Hyperkalaemia:0.43), (PANTOPRAZOLE MAGNESIUM:Hyperkalaemia:1.75), (PARATHYROID HORMONE:Hyperkalaemia:0.02), (PEGINTERFERON ALFA-2B:Hyperkalaemia:0.61), (PEGINTERFERON BETA-1A:Hyperkalaemia:0.66), (PEMETREXED:Hyperkalaemia:0.88), (PENICILLIN G:Hyperkalaemia:0.49), (PERTUZUMAB:Hyperkalaemia:0.41), (PILOCARPINE:Hyperkalaemia: 0.54), (PIMOBENDAN:Hyperkalaemia:1.11), (PIRFENIDONE:Hyperkalaemia:0.17), (POLYE THYLENE GLYCOL 4000:Hyperkalaemia:0.61), (PONATINIB:Hyperkalaemia:1.49), (POSACONAZOLE:Hyperkalaemia: 0.78), (PRANLUKAST:Hyperkalaemia:0.08), (PROGESTERONE:Hyperkalaemia:0.36), (QU INAPRILQUINAPRIL HYDROCHLORIDE:Hyperkalaemia:0.7), (RADIATION THERAPY:Hyperkalaemia:0.31), (RALOXIFENE HYDROCHLORIDE:Hyperkalaemia:1.34), (RIZATRIPTAN:Hyperkalaemia:0.16), (RIZATRIPT AN BENZOATE:Hyperkalaemia:1.42), (RUCAPARIB CAMSYLATE:Hyperkalaemia:0.02), (SALMETEROL:Hyperkalaemia:0.16), (SELEXIPAG: Hyperkalaemia:0.19), (SIMEPREVIR:Hyperkalaemia:0.25), (SIPULEUCEL-T:Hyperkalaemia:0.41), (SODIUM OXYBATE:Hyperkalaemia:1.38), (SULINDAC:Hyperkalaemia:0.01), (SUMATRIPTAN:Hyperkalaemia: 1.23), (SUMATRIPTANSUMATRIPTAN SUCCINATE:Hyperkalaemia:1.5), (TAZOBACTAM:Hyperkalaemia:0.16), (TEDUGLUTIDEW ATER:Hyperkalaemia:0.4), (TELOTRISTAT ETHYL:Hyperkalaemia:0.41), (TERBUTALINE:Hyperkalaemia:0.49), (TERIFLUNOMIDE:Hyperkalaemia: 0.4), (TESTOSTERONE UNDECANOATE:Hyperkalaemia:0.13), (THALIDOMIDE:Hyperkalaemia:2), (THEOPHYLLIN E:Hyperkalaemia:0.04), (THYROID, PORCINETHYROID, UNSPECIFIED:Hyperkalaemia:0.15), (TIGECYCLINE:Hyperkalaemia:0.18), (TOFACITINIB CITRATE:Hyperkalaemia:0.09), (TRAMETINIB:Hyperkalaemia:0.33), (TRAMETINIB DIMETHYL SULFOXIDE:Hyperkalaemia:0.09), (VASOPRESSIN:Hyperkalaemia:0.18), (VINORELBINE TARTRATE:Hyperkalaemia:0.45), (ZILEUTON:Hyperkalaemia:0.34), (ZOLMITRIPTAN:Hyperkalaemia: 0.07), (.ALPHA.1-PROTEINASE INHIBITOR HUMAN:Hyperlipidaemia:1.17), (ABOBOTULINUMTOXINA:Hyperlipidaemia:0.78), (ACAM PROSATE:Hyperlipidaemia:0.13), (ACETAMINOPHENBUTALBITALCAFFEINECODEIN E PHOSPHATE:Hyperlipidaemia:0.03), (ACETAMINOPHENCAFFEINEOPIUM:Hyperlipidaemia: 0.85), (ACETAMINOPHENMETHOCARBAMOL:Hyperlipidaemia:0.23), (ACETAMINO PHENPSEUDOEPHEDRINE HYDROCHLORIDE:Hyperlipidaemia:0.53), (ACOTIAMIDE HYDROCHLORIDE:Hyperlipidaemia:0.29), (ADENOSINE TRIPHOSPHATE DISODIUM:Hyperlipidaemia:0.17), (AJMALINE:Hyperlipidaemia:0.22), (ALBUTEROLIPRA TROPIUM BROMIDE:Hyperlipidaemia:0.03), (ALDESLEUKIN:Hyperlipidaemia:0.38), (ALDIOXA:Hyperlipidaemia: 0.14), (ALUMINUM HYDROXIDEMAGNESIUM OXIDEOXETHAZAINE:Hyperlipidaemia:0.08), (ALUMINUM SILICATE:Hyperlipidaemia:1.5), (AMIKACIN:Hyperlipidaemia:0.42), (AMILORIDE:Hyperlipi daemia:0.2), (AMINO ACIDSVITAMINS:Hyperlipidaemia:0.04), (AMLODIPINEPERINDOPRIL:Hyperlipidaemia:10.3), (AMMONIUM LACTATE:Hyperlipidaemia:1.76), (ANHYDROUS CITRIC ACIDASPIRINSODIUM BICARBONATE:Hyperlipidaemia:0.58), (ASPERGILLUS NIGER .ALPHA.-GALACTOSIDASE:Hyperlipidaemia:0.06), (ATRACURIUM BESYLATE:Hyperlipidaemia:0.11), (ATROPINE SULFATE:Hyperlipidaemia:1.19), (AVOCADO OILSOYBEAN OIL:Hyperlipidaemia:0.18), (AZITHROMYCIN OR METHOTREXATE SODIUM:Hyperlipidaemia:0.23), (BACITRACINNEOMYCIN SULFATE:Hyperlipidaemia:0.54), (BAMIFYLLINE HYDROCHLORIDE:Hyperlipidaemia:0.53), (BARNIDIPINE:Hyperlipidaemia:0.16), (BECLO METHASONE DIPROPIONATEFORMOTEROL:Hyperlipidaemia:0.59), (BENFOTIAMINE:Hyperlipidaemia: 0.04), (BENSERAZIDE:Hyperlipidaemia:0.04), (BEPRIDIL HYDROCHLORIDE:Hyperlipidaemia:0.01), (BETAMETHASONE DIPROPIONATECALCIPOTRIENE:Hyperlipidaemia:0.98), (BETAMETHASONE DIPROPIONATECALCIPOTRIENE HYDRATE:Hyperlipidaemia:0.06), (BETAMETHASONE SODIUM PHOSPHATENEOMYCIN SULFATE:Hyperlipidaemia:1.18), (BETAMIPRONPANIPENEM:Hyperlipidaemia:0.28), (BET HANECHOL CHLORIDE:Hyperlipidaemia:1.47), (BICARBONATE IONPOLYETHYLENE GLYCOLSPOTASSIUM CATIONSODIUM CHLORIDESODIUM SULFATE ANHYDROUS:Hyperlipidaemia:0.68), (BISACODYLGLYCERINMINERAL OILSODIUM PHOSPHATE, DIBASICSODIUM PHOSPHATE, MONOBASIC:Hyperlipidaemia:0.04), (BISMUTH SUBSALICYLATECALCIUM CARBONATE:Hyperlipidaemia:0.14), (BRIVARACETAM:Hyperlipidaemia:0.38), (BUCOLO ME:Hyperlipidaemia:0.6), (BUFORMIN:Hyperlipidaemia:1.11), (CABAZITAXEL:Hyperlipidae mia:0.82), (CABOZANTINIB:Hyperlipidaemia:0.39), (CABOZANTINIB S-MALATE:Hyperlipidaemia:0.59), (CALCIUM PANTOTHENATECYANOCOBALAMINNIACINAMIDEPYRIDOXINE HYDROCHLORIDERIBOFLAVINTHIAMINE MONONITRATE:Hyperlipidaemia:0.56), (CALCIUM PHOSPHATE, DIBASIC, ANHYDROUSFERROUS SUCCINATEMAGNESIUM HYDROXIDE:Hyperlipidaemia:0.01), (CALCIUMMAGNESIUMZINC:Hyperlipidaemia:1.7), (CAMPHORCAPSICUMMETHYL SALICYLATE:Hyperlipidaemia:0.48), (CAMPHORDIPHENHYDRAMINEZINC OXIDE:Hyperlipidaemia:0.27), (CANNABIDIOL:Hyperlipidaemia:0.16), (CANRENOATE POTASSIUM:Hyperlipidaemia:0.8), (CARBASALATE:Hyperlipidaemia:0.57), (CARBASPIRIN:Hyperlipidaemia:0.01), (CARMUSTINE:Hyperlipidaemia:0.07), (CASPOFUNGIN:Hyperlipidae mia:0.54), (CEFMETAZOLE:Hyperlipidaemia:0.02), (CEFOTAXIMECEFOTAXIME SODIUM:Hyperlipidaemia:0.43), (CEFOTIAM HYDROCHLORIDE:Hyperlipidaemia:0.77), (CEFPROZIL:Hyperlipidaemia:0.06), (CERIVAST ATIN SODIUM:Hyperlipidaemia:0.72), (CETIRIZINE HYDROCHLORIDEPSEUDOEPHEDRINE HYDROCHLORIDE:Hyperlipidaemia:1.17), (CHLOROTHIAZIDE:Hyperlipidaemia:1.03), (CH LORPHENESIN CARBAMATE:Hyperlipidaemia:0.04), (CHLORPROMAZINE HYDROCHLORIDE:Hyperlipidaemia:0.29), (CHOLINE BITARTRATECYSTEINEINOSITOLMAMMAL LIVER:Hyperlipidaemia:0.86), (CISATRACURIUM:Hyperlipidaemia:0.5), (CITICOLINE:Hype rlipidaemia:0.08), (CLIKSTAR:Hyperlipidaemia:1.2), (COBICISTATELVITEGRAVIREMTRI CITABINETENOFOVIR ALAFENAMIDE FUMARATE:Hyperlipidaemia:0.27), (COLCHICINEDICYCLOMINE HYDROCHLORIDE:Hyperlipidaemia:0.03), (COLISTIMETHATE SODIUM:Hyperlipidaemia:0.18), (COLISTIN:Hyperlipidaemia:0.06), (COLLAGEN:Hyperlipida emia:0.23), (CORTICOTROPIN:Hyperlipidaemia:1.82), (CUPRIC SULFATEFERRIC CHLORIDEMANGANESE CHLORIDEPOTASSIUM IODIDEZINC SULFATE:Hyperlipidaemia:0.21), (CYAMEMAZINE:Hyperlipidaemia:0.56), (CYANAMIDE:H yperlipidaemia:0.38), (CYANOCOBALAMINPYRIDOXAMINE HYDROCHLORIDETHIAMINE HYDROCHLORIDE:Hyperlipidaemia:0), (CYPROHEPTADINE:Hyperlipidaemia:1.81), (CYPR OTERONE:Hyperlipidaemia:0.12), (DACLATASVIR DIHYDROCHLORIDE:Hyperlipidaemia:0.03), (DACLIZUMAB:Hyperlipidaemia:2.21), (DAN AZOL:Hyperlipidaemia:1.8), (DANTROLENE SODIUM:Hyperlipidaemia:1.25), (DEFERIPRONE:Hyperlipidaemia:0.68), (DEFLAZACORT:H yperlipidaemia:2.39), (DEMECLOCYCLINE:Hyperlipidaemia:0.07), (DENOPAMINE:Hyperlipi daemia:0.19), (DEXMETHYLPHENIDATE HYDROCHLORIDE:Hyperlipidaemia:0.28), (DEXPANTHENOLNIACINAMIDEPYRIDOXI NE HYDROCHLORIDERIBOFLAVIN 5′-PHOSPHATE SODIUMTHIAMINE HYDROCHLORIDE:Hyperlipidaemia:0.34), (DEXTROAMPHETAMINE SULFATE:Hyperlipidaemia:1.21), (DEXTROSEPOTASSIUM CHLORIDESODIUM CHLORIDESODIUM LACTATE:Hyperlipidaemia:0.7), (DIASTASE:Hyperlipidaemia:0.08), (DIFLUNISAL:Hyperlipi daemia:0.81), (DIGITALIS:Hyperlipidaemia:0.48), (DIOSMIN:Hyperlipidaemia:0.81), (DIPYRO NEPROMETHAZINE HYDROCHLORIDE:Hyperlipidaemia:0.25), (DOLASETRON MESYLATE:Hyperlipidaemia:1.19), (DORIPENEM:Hyperlipidaemia:0.14), (DOXYLAMINE SUCCINATEPYRIDOXINE HYDROCHLORIDE:Hyperlipidaemia:0.17), (DYDROGESTERONE:Hyperlipidaemia:0.33), (E CABET SODIUM:Hyperlipidaemia:1.45), (ELBASVIR:Hyperlipidaemia:0.79), (EPHEDRINE HYDROCHLORIDE:Hyperlipidaemia:0.84), (ERGOLOID MESYLATES:Hyperlipidaemia:0.08), (ERIBULIN:Hyperlipidaemia:0.67), (ERYTHROMYCIN SULFISOXAZOLE:Hyperlipidaemia:0.3), (ESTRADIOL BENZOATE:Hyperlipidaemia:0.02), (ESTRADIOLNORETHINDRONE ACETATE:Hyperlipidaemia:0.78), (ESTRAMUSTINE PHOSPHATE SODIUM:Hyperlipidaemia:0.13), (EUCALYPTOLMENTHOLMETHYL SALICYLATETHYMOL:Hyperlipidaemia:0.13), (EVENING PRIMROSE OIL:Hyperlipidaemia:0.1), (FELBAMATE:Hyperlipidaemia:0.41), (FERROUS BISGLYCINATE:Hyperlipidaemia:0.86), (FERROUS GLYCINE SULFATE:Hyperlipidaemia:1.04), (FLOPROPIONE:Hyperlipidaemia:0.43), (FLUNARIZINE HYDROCHLORIDE:Hyperlipidaemia:0.39), (FLUOXETINE HYDROCHLORIDEOLANZAPINE:Hyperlipidaemia:0.41), (FLUPHENAZINE:Hyperlipidaem ia:0.77), (FOSFOMYCIN:Hyperlipidaemia:0.48), (FOSPHENYTOIN SODIUM:Hyperlipidaemia:0.32), (FROVATRIPTAN SUCCINATE:Hyperlipidaemia:0.18), (FUROSEMIDESPIRONOLACTONE:Hyperlipidaemia:00.4), (GLUCONATE:Hyperlipidaemia:0.04), (GLYCOPYRROLATEINDACATEROL MALEATE:Hyperlipidaemia:1.27), (GRAZOPREVIR:Hyperlipidaemia:0.76), (GUAIAZULENE:Hyperlipidaemia:0.38), (HALOPERIDOL DECANOATE:Hyperlipidaemia:0.62), (HIGH DENSITY POLYETHYLENE:Hyperlipidaemia:0.12), (HORSE CHESTNUT:Hyperlipidaemia:0.05), (HUMAN PLASMA:Hyperlipidaemia:0.15), (HYALURONIC ACID:Hyperlipidaemia:0.34), (HYDROCHLOROTHIAZIDEMETOPROLOL TARTRATE:Hyperlipidaemia:0.03), (HYDROCORTISONE PROBUTATE:Hyperlipidaemia:0.78), (HYDROCORTISONE SODIUM PHOSPHATE:Hyperlipidaemia:0.16), (IBRITUMOMAB TIUXETAN:Hyperlipidaemia:0.76), (IFENPRODIL TARTRATE:Hyperlipidaemia:0.79), (IMIGLUCERASE:Hyperlipidaemia:1.33), (INDACATER OL MALEATE:Hyperlipidaemia:0.14), (INFLIXIMAB-DYYB:Hyperlipidaemia:0.93), (INSULIN ASPARTINSULIN DEGLUDEC:Hyperlipidaemia:1.5), (INSULIN PORKINSULIN PURIFIED PORK:Hyperlipidaemia:0.9), (INTERFERON:Hyperlipidaemia:1.99), (INTERFERON ALFA-2A:Hyperlipidaemia:1.55), (INTERFERON ALFA-2B:Hyperlipidaemia:1.46), (INTERFERON ALFA-N1:Hyperlipidaemia:0.19), (INTERFERON GAMMA-1B:Hyperlipidaemia:0.63), (IRBESARTANTRICHLORMETHIAZIDE:Hyperlipidaemia:0.44), (IRON POLYMALTOSE:Hyperlipidaemia:0.06), (IRONVITAMINS:Hyperlipidaemia:0.02), (ITRACO NAZOLESODIUM CHLORIDE:Hyperlipidaemia:0.54), (LANDIOLOL HYDROCHLORIDE:Hyperlipidaemia:1.37), (LANREOTIDE:Hyperlipidaemia:0.91), (LAPATI NIB DITOSYLATE:Hyperlipidaemia:1.23), (LECITHINSODIUM BICARBONATESODIUM PHOSPHATE, MONOBASIC:Hyperlipidaemia:0.23), (LENVATINIB:Hyperlipidaemia:1.09), (LEVOMEPROM AZINE MALEATE:Hyperlipidaemia:0.11), (LEVOSIMENDAN:Hyperlipidaemia:0.7), (LINSIDOMINE: Hyperlipidaemia:0.74), (LUTETIUM LU-177:Hyperlipidaemia:0.7), (MAGALDRATE:Hyperlipidaemia:0.1), (MAGNESIUM ASPARTATEPOTASSIUM ASPARTATE:Hyperlipidaemia:0.53), (MAGNESIUM PIDOLATE:Hyperlipidaemia:0.29), (MENTHOLZINC OXIDE:Hyperlipidaemia:0.29), (MEPENZOLATE BROMIDE:Hyperlipidaemia:0.92), (MEPOLIZUMAB:Hyperlipidaemia:1.42), (METFORMIN HYDROCHLORIDESAXAGLIPTIN:Hyperlipidaemia:1.01), (METFORMIN PAMOATE:Hyperlipidaemia:0.96), (METHENAMINE MANDELATE:Hyperlipidaemia:0.47), (METHENOLONE ACETATE:Hyperlipidaemia:0.45), (METYRAPONE:Hyperlipidaemia:0.08), (MIGLUSTAT:Hy perlipidaemia:0.7), (MILRINONE:Hyperlipidaemia:0.44), (MIRIPLATIN:Hyperlipidaemia:0.22), (NALBUPHINE HYDROCHLORIDE:Hyperlipidaemia:0.1), (NALDEMEDINE:Hyperlipidaemia:0.46), (NAPRO XEN SODIUMSUMATRIPTAN SUCCINATE:Hyperlipidaemia:0.09), (NEFAZODONE:Hyperlipidaemia:0.09), (NEFAZODONE HYDROCHLORIDE:Hyperlipidaemia:0.36), (NEOMYCIN:Hyperlipidaemia:0.38), (OCTOTIA MINE:Hyperlipidaemia:0.18), (OK-432:Hyperlipidaemia:0.26), (OLANZAPINE PAMOATE:Hyperlipidaemia:0.09), (OLOPATADINE:Hyperlipidaemia:0.76), (ORYCTOLAGU S CUNICULUS SKIN:Hyperlipidaemia:0.18), (OXYBUTYNINOXYBUTYNIN CHLORIDE:Hyperlipidaemia:1.24), (OZAGREL:Hyperlipidaemia:0.16), (PAZUFLOXACIN MESILATE:Hyperlipidaemia:0.56), (PEGVISOMANT:Hyperlipidaemia:0.28), (PENICILLIN G:Hyperlipidaemia:1.34), (PENTOSTATIN:Hyperlipidaemia:0.85), (PEROSPIRONE:Hyperlipid aemia:0.6), (PIZOTYLINE:Hyperlipidaemia:0.11), (POLYMYXIN B SULFATE:Hyperlipidaemia:0.07), (PORFIMER SODIUM:Hyperlipidaemia:0.26), (POTASSIUM IODIDE:Hyperlipidaemia:1.26), (POVIDONE:Hyperlipidaemia:0.92), (PRAMOXINE HYDROCHLORIDE:Hyperlipidaemia:0.49), (PREGNENOLONE:Hyperlipidaemia:0.12), (PRIS TINAMYCIN:Hyperlipidaemia:0.88), (PROCYCLIDINE:Hyperlipidaemia:0.53), (PROMAZINE HYDROCHLORIDE:Hyperlipidaemia:0.02), (PROPARACAINE:Hyperlipidaemia:0.06), (PROP ATYL NITRATE:Hyperlipidaemia:0.53), (PROTRIPTYLINE HYDROCHLORIDE:Hyperlipidaemia:0.98), (PRUCALOPRIDE SUCCINATE:Hyperlipidaemia:0.39), (PYRIDOXAL PHOSPHATE:Hyperlipidaemia:0.63), (QUERCUS SALICINA STEM:Hyperlipidaemia:0.64), (QUINACRINE:Hyperlipidaemia:1.01), (RADIATION THERAPY:Hyperlipidaemia:1.97), (RAMOSETRON HYDROCHLORIDE:Hyperlipidaemia:0.3), (RILMENIDINE PHOSPHATE:Hyperlipidaemia:0.86), (ROFECOXIB:Hyperlipidaemia:1), (ROXADUSTAT:Hyp erlipidaemia:0.06), (RUCAPARIB CAMSYLATE:Hyperlipidaemia:0.32), (RUPATADINE FUMARATE:Hyperlipidaemia:0.1), (SELEGILINE:Hyperlipidaemia:1.22), (SENNOSIDE A:Hyperlipidaemia:0.36), (SIBUTRAMINE HYDROCHLORIDE:Hyperlipidaemia:0.5), (SILICON DIOXIDE:Hyperlipidaemia:0.28), (SIVELESTAT SODIUM:Hyperlipidaemia:0.94), (SODIUM BICARBONATESODIUM CHLORIDE:Hyperlipidaemia:0.08), (SODIUM CITRATE:Hyperlipidaemia:0.83), (SODIUM PHOSPHATE, DIBASIC:Hyperlipidaemia:0.12), (STREPTOMYCIN SULFATE:Hyperlipidaemia:0.11), (SUFENTANIL:Hyperlipidaemia:0.66), (SUFENTANIL CITRATE:Hyperlipidaemia:0.92), (SULTOPRIDE HYDROCHLORIDE:Hyperlipidaemia:0.44), (SUNITINIB:Hyperlipidaemia:0.09), (SUPLATAST TOSILATE:Hyperlipidaemia:2.35), (TABALUMAB:Hyperlipidaemia:0.28), (TECELEUKIN:Hy perlipidaemia:0.07), (TEDUGLUTIDEWATER:Hyperlipidaemia:1.02), (TELOTRISTAT ETHYL:Hyperlipidaemia:0.66), (TETRACAINE HYDROCHLORIDE:Hyperlipidaemia:0.33), (THIOPENTAL:Hyperlipidaemia:0.96), (THIOTHI XENE:Hyperlipidaemia:0.01), (TIANEPTINE:Hyperlipidaemia:0.04), (TIAPRIDE:Hyperlipidae mia:0.08), (TIBOLONE:Hyperlipidaemia:0.24), (TILIDINE:Hyperlipidaemia:0.61), (TIMOLOL MALEATETRAVOPROST:Hyperlipidaemia:1.66), (TIMOLOLTRAVOPROST:Hyperlipidae mia:0.9), (TINZAPARIN:Hyperlipidaemia:1.31), (TOFACITINIB:Hyperlipidaemia:0.08), (TORE MIFENE CITRATE:Hyperlipidaemia:0.4), (TRAMETINIB DIMETHYL SULFOXIDE:Hyperlipidaemia:0.57), (TRIMEPRAZINE:Hyperlipidaemia:0.01), (TRIPTORELI N PAMOATE:Hyperlipidaemia:1.03), (TULOBUTEROL HYDROCHLORIDE:Hyperlipidaemia:0.25), (UROKINASE:Hyperlipidaemia:1.41), (VECURO NIUM BROMIDE:Hyperlipidaemia:0.86), (VINORELBINEVINORELBINE TARTRATE:Hyperlipidaemia:0.59), (ZEAXANTHIN:Hyperlipidaemia:0.08), (ZIV-AFLIBERCEPT:Hyperlipidaemia:0.22), (ZUCLOPENTHIXOL DECANOATE:Hyperlipidaemia:0.45), (CALCIUM CARBONATECHOLECALCIFEROL:Hyperparathyroidism:0.58), (CANDESARTAN CILEXETILHYDROCHLOROTHIAZIDE:Hyperparathyroidism:0.22), (CELECOXIB:Hyperpa rathyroidism:0.26), (DICLOFENAC:Hyperparathyroidism:0.14), (ENOXAPARIN:Hyperparathyr oidism:0.02), (EVOLOCUMAB:Hyperparathyroidism:0.52), (IBRUTINIB:Hyperparathyroidism: 0.84), (ILOPROST:Hyperparathyroidism:0.06), (LEDIPASVIRSOFOSBUVIR:Hyperparathyroid ism:0.46), (LORATADINE:Hyperparathyroidism:0.03), (LOVASTATIN:Hyperparathyroidism:0.13), (MELOXICAM:Hyperparathyroidism:0.17), (NAPROXEN:Hyperparathyroidism:0.64), (OX YGEN:Hyperparathyroidism:0.12), (POLYETHYLENE GLYCOLS:Hyperparathyroidism:0.13), (TERIFLUNOMIDE:Hyperparathyroidism:1.02), (ALB UTEROL SULFATEIPRATROPIUM BROMIDE:Hyperparathyroidism secondary:0.08), (BISACODYL OR DOCUSATE SODIUM:Hyperparathyroidism secondary:0.33), (BUMETANIDE:Hyperparathyroidism secondary:0.61), (CANAGLIFLOZIN:Hyperparathyroidism secondary:0.02), (CANDESARTAN CILEXETILHYDROCHLOROTHIAZIDE:Hyperparathyroidism secondary:0.48), (CARVEDILOL PHOSPHATE:Hyperparathyroidism secondary:0.07), (CIPROFLOXACIN HYDROCHLORIDE:Hyperparathyroidism secondary:0.03), (CLOTRIMAZOLE:Hyperparathyroidism secondary:0.4), (DABIGATRAN:Hyperparathyroidism secondary:0.3), (DEXLANSOPRAZOLE:Hyperparathyroidism secondary:0.42), (EDOXABAN TOSYLATE:Hyperparathyroidism secondary:1.22), (ELDECALCITOL:Hyperparathyroidism secondary:0.03), (EPINEPHRINE:Hyperparathyroidism secondary:0.12), (EPLERENONE:Hyperparathyroidism secondary:0.37), (EVOLOCUMAB:Hyperparathyroidism secondary:0.19), (GADOLINIUM:Hyperparathyroidism secondary:0.3), (GUANABENZ ACETATE:Hyperparathyroidism secondary:0), (HYDROCHLOROTHIAZIDEIRBESARTAN:Hyperparathyroidism secondary:0.08), (HYDROCHLOROTHIAZIDELOSARTAN POTASSIUM:Hyperparathyroidism secondary:0.11), (HYDROCHLOROTHIAZIDEVALSARTAN:Hyperparathyroidism secondary:0.32), (IBRUTINIB:Hyperparathyroidism secondary:0.45), (ILOPROST:Hyperparathyroidism secondary:0.24), (IMATINIB MESYLATE:Hyperparathyroidism secondary:0.33), (LEDIPASVIRSOFOSBUVIR:Hyperparathyroidism secondary:0.54), (LERCANIDIPINE:Hyperparathyroidism secondary:0.57), (LERCANIDIPINE HYDROCHLORIDE:Hyperparathyroidism secondary:0.33), (METHADONE HYDROCHLORIDE:Hyperparathyroidism secondary:0.1), (METHYLPREDNISOLONE SODIUM SUCCINATE:Hyperparathyroidism secondary:0.17), (METILDIGOXIN:Hyperparathyroidism secondary:0.2), (METRONIDAZOLE:Hyperparathyroidism secondary:0.07), (NAPROXEN:Hyperparathyroidism secondary:0.34), (NILOTINIB:Hyperparathyroidism secondary:0.24), (OXCARBAZEPINE:Hyperparathyroidism secondary:0.18), (PHENOBARBITAL:Hyperparathyroidism secondary:0.15), (PILSICAINIDE HYDROCHLORIDE:Hyperparathyroidism secondary:0.57), (PIMOBENDAN:Hyperparathyroidism secondary:0.68), (POLYETHYLENE GLYCOLS:Hyperparathyroidism secondary:0.39), (PRIMIDONE:Hyperparathyroidism secondary:0.26), (PROMETHAZINEPROMETHAZINE HYDROCHLORIDE:Hyperparathyroidism secondary:0.11), (SACUBITRILVALSARTAN:Hyperparathyroidism secondary:0.02), (SITAGLIPTIN:Hyperparathyroidism secondary:0.56), (SODIUM PHOSPHATE, DIBASICSODIUM PHOSPHATE, MONOBASIC:Hyperparathyroidism secondary:0.01), (SOTALOL:Hyperparathyroidism secondary:0.6), (TIOTROPIUM BROMIDE MONOHYDRATE:Hyperparathyroidism secondary:0.68), (TOPIRAMATE:Hyperparathyroidism secondary:0.59), (VENLAFAXINE HYDROCHLORIDE:Hyperparathyroidism secondary:0.39), (ACETAZOLAMIDE:Hyperphosphataemia:0.31), (ALENDRONIC ACID:Hyperphosphataemia:0.39), (ANASTROZOLE:Hyperphosphataemia:0.41), (BENIDIPINE: Hyperphosphataemia:0.09), (BERAPROST:Hyperphosphataemia:0.01), (BUDESONIDE:Hyperp hosphataemia:1.2), (CALCIUM CARBONATECHOLECALCIFEROLMAGNESIUM CARBONATE:Hyperphosphataemia:0.03), (CALCIUM CITRATE:Hyperphosphataemia:0.01), (CANAGLIFLOZIN:Hyperphosphataemia:0.31), (CAPT OPRIL:Hyperphosphataemia:0.32), (CARFILZOMIB:Hyperphosphataemia:0.22), (CARVEDILO L PHOSPHATE:Hyperphosphataemia:0.29), (CEFAZOLIN:Hyperphosphataemia:0.35), (CERTOL IZUMAB PEGOL:Hyperphosphataemia:0.32), (CIFENLINE:Hyperphosphataemia:0.25), (CIPROFLOXAC IN HYDROCHLORIDE:Hyperphosphataemia:0.32), (CLOTRIMAZOLE:Hyperphosphataemia:0.72), (CYCLOBENZAPRINE:Hyperphosphataemia:0.19), (DABIGATRAN:Hyperphosphataemia:0.55), (DANAZOL:Hyperphosphataemia:0.17), (DASATINIB:Hyperphosphataemia:0.42), (DESLO RATADINE:Hyperphosphataemia:0.06), (DEXAMETHASONE ACETATE:Hyperphosphataemia:0.45), (DEXLANSOPRAZOLE:Hyperphosphataemia:0.3), (DI CLOFENAC:Hyperphosphataemia:0.09), (DIGITOXIN:Hyperphosphataemia:0.15), (DIPHENID OL HYDROCHLORIDE:Hyperphosphataemia:0.23), (DOBUTAMINE HYDROCHLORIDE:Hyperphosphataemia:0.06), (DOXYCYCLINE:Hyperphosphataemia:0.17), (DUTASTERIDE:Hyperphosphataemia:0.56), (EDOXABAN TOSYLATE:Hyperphosphataemia:1.7), (EPINEPHRINE:Hyperphosphataemia:0.65), (EPLEREN ONE:Hyperphosphataemia:0.67), (ETRETINATE:Hyperphosphataemia:0.5), (EVOLOCUMAB: Hyperphosphataemia:0.26), (EZETIMIBESIMVASTATIN:Hyperphosphataemia:0.01), (FESOT ERODINE FUMARATE:Hyperphosphataemia:0.18), (FLECAINIDE:Hyperphosphataemia:0.73), (FURSUL TIAMINE:Hyperphosphataemia:0.31), (GLYBURIDE:Hyperphosphataemia:0.39), (GLYCOPYR RONIUMINDACATEROL:Hyperphosphataemia:0.03), (HYDROCHLOROTHIAZIDETELMI SARTAN:Hyperphosphataemia:0.12), (HYDROCHLOROTHIAZIDEVALSARTAN:Hyperpho sphataemia:0.44), (IBRUTINIB:Hyperphosphataemia:0.71), (ILOPROST:Hyperphosphataemia:0.3), (IMATINIB MESYLATE:Hyperphosphataemia:0.29), (IMIPENEM:Hyperphosphataemia:0.09), (INDAPAMI DEPERINDOPRIL:Hyperphosphataemia:0.19), (IPRAGLIFLOZIN L-PROLINE:Hyperphosphataemia:0.06), (LEDIPASVIRSOFOSBUVIR:Hyperphosphataemia:0.9), (LERCANIDIPINE HYDROCHLORIDE:Hyperphosphataemia:0.36), (LEVALBUTEROL HYDROCHLORIDE:Hyperphosphataemia:0.46), (METHADONE HYDROCHLORIDE:Hyperphosphataemia:0.38), (METHENOLONE ENANTHATE:Hyperphosphataemia:0.13), (METILDIGOXIN:Hyperphosphataemia:0.72), (MIN OCYCLINEMINOCYCLINE HYDROCHLORIDE:Hyperphosphataemia:0.03), (MINODRONIC ACID:Hyperphosphataemia:0.6), (MOXONIDINE:Hyperphosphataemia:0.19), (NALOXONE HYDROCHLORIDEOXYCODONE HYDROCHLORIDE:Hyperphosphataemia:0.12), (NEBIVOLOL HYDROCHLORIDE:Hyperphosphataemia:0.25), (NIACIN:Hyperphosphataemia:0.05), (NICAR DIPINE:Hyperphosphataemia:0.13), (NILOTINIB:Hyperphosphataemia:0.98), (OXCARBAZEPI NE:Hyperphosphataemia:0.32), (PATIROMER:Hyperphosphataemia:0.11), (PENICILLIN:Hyper phosphataemia:0.02), (PONATINIB:Hyperphosphataemia:0.44), (PRAMIPEXOLE DIHYDROCHLORIDE:Hyperphosphataemia:0.27), (PRIMIDONE:Hyperphosphataemia:0.4), (P ROBUCOL:Hyperphosphataemia:0.05), (PROMETHAZINEPROMETHAZINE HYDROCHLORIDE:Hyperphosphataemia:0.6), (SACUBITRILVALSARTAN:Hyperphosphata emia:0.3), (SODIUM PHOSPHATE, DIBASICSODIUM PHOSPHATE, MONOBASIC:Hyperphosphataemia:0.17), (SULFAMETHOXAZOLE:Hyperphosphataemia:0.22), (TERIFLUNOMIDE:Hyperphosphataemia:0.05), (TIOTROPIUM:Hyperphosphataemia:0.14), (TOPIRAMATE:Hyperphosphataemia:0.66), (.ALPHA.-TOCOPHEROL ACETATE, D-ASCORBIC ACIDCALCIUM PANTOTHENATEFOLIC ACIDMANGANESENIACINAMIDEPYRIDOXINERIBOFLAVINTHIAMINEVITAMIN AZINC OXIDE:Hypersensitivity:0.17), (.ALPHA.-TOCOPHEROLVITAMINS:Hypersensitivity:0.85), (.BETA.-CAROTENE:Hypersensitivity:0.22), (.BETA.-HYDROXYISOVALERIC ACIDARGININEGLUTAMINE:Hypersensitivity:0.08), (ABACAVIR SULFATELAMIVUDINE:Hypersensitivity:0.9), (ACECLOFENAC:Hypersensitivity:0.57), (AC ER NEGUNDO POLLEN:Hypersensitivity:0.89), (ACETAMINOPHENCAFFEINECARISOPRODOLDICLO FENAC:Hypersensitivity:0.68), (ACETAMINOPHENCHLORPHENIRAMINE MALEATEPSEUDOEPHEDRINE HYDROCHLORIDE:Hypersensitivity:0.67), (ACETAMINOPHENMETHOCARBAMOL:Hyp ersensitivity:1.04), (ALBUTEROLGUAIFENESIN:Hypersensitivity:1), (ALBUTEROLIPRAT ROPIUM BROMIDE:Hypersensitivity:0.93), (ALFALFA:Hypersensitivity:0.54), (ALISKIREN HEMIFUMARATEHYDROCHLOROTHIAZIDE:Hypersensitivity:0.54), (ALISKIRENHYDR OCHLOROTHIAZIDE:Hypersensitivity:0.3), (ALLERGENIC EXTRACT-CANDIDA:Hypersensitivity:0.02), (ALTERNARIA ALTERNATA ASPERGILLUS NIGER VAR. NIGERCOCHLIOBOLUS SATIVUSCLADOSPORIUM SPHAEROSPERMUMPENICILLIUM CHRYSOGENUM VAR. CHRYSOGENUM:Hypersensitivity:0.24), (ALUMINUM CHLORIDE:Hypersensitivity:0.51), (ALUMINUM HYDROXIDEMAGNESIUM OXIDEOXETHAZAINE:Hypersensitivity:0.16), (AMARANTHUS RETROFLEXUS POLLENCHENOPODIUM ALBUM POLLENXANTHIUM STRUMARIUM POLLEN:Hypersensitivity:0.65), (AMILORIDE HYDROCHLORIDEFUROSEMIDE:Hypersensitivity:0.75), (AMINOLEVULINIC ACID HYDROCHLORIDE:Hypersensitivity:0.23), (AMISULPRIDE:Hypersensitivity:1.39), (AMITRI PTYLINECHLORDIAZEPOXIDE:Hypersensitivity:0.16), (AMLODIPINELOSARTAN:Hyper sensitivity:0.09), (AMMONIUM CHLORIDEDEXTROMETHORPHANSODIUM CITRATE:Hypersensitivity:1.45), (ANHYDROUS CITRIC ACIDMAGNESIUM OXIDESODIUM PICOSULFATE:Hypersensitivity:0.26), (ANIDULAFUNGIN:Hypersensitivity:0.16), (ARIPIPR AZOLE LAUROXIL:Hypersensitivity:1.53), (ASCORBIC ACID/CHROMIUM/SELENIUM/VANADYL SULFATE:Hypersensitivity:1.37), (ASCORBIC ACIDFERROUS SULFATEFOLIC ACIDPYRIDOXINE HYDROCHLORIDERIBOFLAVINTHIAMINE MONONITRATE:Hypersensitivity:0.3), (ASCORBIC ACIDPOLYETHYLENE GLYCOL 3350\POTASSIUM CHLORIDESODIUM ASCORBATESODIUM CHLORIDESODIUM SULFATE:Hypersensitivity:0.07), (ASPARAGINASE:Hypersensitivity:0.29), (ASPIRINDIHY DROXYALUMINUM AMINOACETATE ANHYDROUSMAGNESIUM CARBONATE:Hypersensitivity:1.37), (ATENOLOLHYDROCHLOROTHIAZIDE:Hypersensit ivity:0.18), (ATROPINE SULFATEDIPHENOXYLATE:Hypersensitivity:0.13), (AUREOBASIDIUM PULLULANS VAR. PULLUTANSCOCHLIOBOLUS SPICIFERGIBBERELLA FUJIKUROIMUCOR PLUMBEUSRHIZOPUS STOLONIFER:Hypersensitivity:0.62), (AZITHROMYCIN OR METHOTREXATE SODIUM:Hypersensitivity:0.4), (BACITRACIN ZINC:Hypersensitivity:0.46), (BACITRACINNEOMYCINPOLYMYXIN B:Hypersensitivity:0.87), (BELATACEPT:Hypersensitivity:0.67), (BERAPROST:Hypersensitivit y:0.31), (BETAMETHASONE DIPROPIONATECALCIPOTRIENE HYDRATE:Hypersensitivity:0.46), (BETULA NIGRA POLLENCARYA OVATA POLLENFAGUS GRANDIFOLIAFRAXINUS AMERICANA POLLEN POPULUS DELTOIDES POLLENQUERCUS RUBRA POLLENULMUS AMERICANA POLLEN:Hypersensitivity:0.65), (BEXAROTENE:Hypersensitivity:0.01), (BIPERIDEN HYDROCHLORIDE:Hypersensitivity:0.2), (BORAGE OIL:Hypersensitivity:0.11), (BORON:Hypersensitivity:0.45), (BRINZOLAMIDETIMOLOL MALEATE:Hypersensitivity:0.44), (BRIVARACETAM:Hypersensitivity:0.55), (BROMHEXIN E:Hypersensitivity:0.45), (BUPRENORPHINENALOXONE:Hypersensitivity:0.81), (BUPROPI ON HYDROBROMIDE:Hypersensitivity:0.5), (BUTORPHANOL:Hypersensitivity:0.79), (CABOZA NTINIB:Hypersensitivity:0.01), (CAFFEINEERGOTAMINE TARTRATE:Hypersensitivity:0.04), (CAFFEINEISOMETHEPTENEMETHIMAZOLE:Hyper sensitivity:0.16), (CAFFEINEMAGNESIUM SALICYLATE:Hypersensitivity:0.28), (CAJUPUT OILEUCALYPTUS OILPEPPERMINT OIL:Hypersensitivity:0.52), (CALCIUM ASCORBATE:Hypersensitivity:0.1), (CALCIUM BICARBONATE:Hypersensitivity:0.33), (CALCIUM CARBONATEETIDRONATE DISODIUM:Hypersensitivity:0.66), (CALCIUM PANTOTHENATECUPRIC SULFATECYANOCOBALAMINFERROUS ASPARTO GLYCINATEFERROUS FUMARATEFOLIC ACIDMAGNESIUM SULFATENIACINAMIDEPYRIDOXINE HYDROCHLORIDERIBOFLAVINSODIUM ASCORBATETHIAMINE MONONITRATEZINC SULFATE:Hypersensitivity:0.05), (CALCIUM PANTOTHENATECYANOCOBALAMINNIACINAMIDEPYRIDOXINE HYDROCHLORIDERIBOFLAVINTHIAMINE MONONITRATE:Hypersensitivity:1.11), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:Hypersensitivity:0.25), (CAPSICUM:Hypersensitivity:0.44), (CARMUSTINE:Hy persensitivity:0.61), (CARTEOLOL:Hypersensitivity:0.38), (CARTEOLOL HYDROCHLORIDE:Hypersensitivity:0.51), (CARYA OVATA POLLEN:Hypersensitivity:0.65), (CEFOTAXIMECEFOTAXIME SODIUM:Hypersensitivity:0.72), (CEFTAZIDIME SODIUM:Hypersensitivity:0.5), (CERIVASTATIN SODIUM:Hypersensitivity:1.01), (CERTOPARIN SODIUM:Hypersensitivity:0.21), (CHLORAMPHENICOLPREDNISOLONE:Hypersensitivity: 0.33), (CHLORPROTHIXENE:Hypersensitivity:0.15), (CHOLECALCIFEROLGENISTEINZI NC GLYCINATE:Hypersensitivity:0.15), (CHOLINETRAZODONE HYDROCHLORIDE:Hypersensitivity:0.23), (CILNIDIPINE:Hypersensitivity:0.01), (CIPROFIB RATE:Hypersensitivity:1.35), (CIPROFLOXACIN HYDROCHLORIDEHYDROCORTISONE:Hypersensitivity:0.25), (CITRIC ACID MONOHYDRATEPOTASSIUM CITRATE:Hypersensitivity:0.49), (CLADRIBINE:Hypersensitivity:0.1), (CLOBETASONE BUTYRATE:Hypersensitivity:0.04), (CLONIXINCYCLOBENZAPRINE:Hypersensitivity:0.28), (COAL TAR:Hypersensitivity:0.01), (COBICISTATDARUNAVIR ETHANOLATE:Hypersensitivity:0.17), (CODEINE PHOSPHATEPROMETHAZINE:Hypersensitivity:0.53), (CROTALIDAE POLYVALENT IMMUNE FAB (OVINE):Hypersensitivity:0.56), (CYANOCOBALAMINDICLOFENAC SODIUMPYRIDOXINE HYDROCHLORIDETHIAMINE MONONITRATE:Hypersensitivity:0.74), (CYCLOPHOSPHAMIDECYTARABINEDEXAME THASONEDOXORUBICINMETHOTREXATEVINCRISTINE:Hypersensitivity:0.08), (CYP ROTERONE ACETATEETHINYL ESTRADIOL:Hypersensitivity:0.62), (CYSTEAMINE BITARTRATE:Hypersensitivity:0.09), (DABRAFENIB MESYLATE:Hypersensitivity:0.82), (DANTROLENE SODIUM:Hypersensitivity:1.23), (DEFERIPRONE:Hypersensitivity:0.36), (DEGARELIX:Hyper sensitivity:0.21), (DEOXYRIBONUCLIEC ACID:Hypersensitivity:0.48), (DEXAMETHASONE ACETATE:Hypersensitivity:1.41), (DEXAMETHASONENEOMYCINPOLYMYXIN B SULFATE:Hypersensitivity:0.29), (DEXMEDETOMIDINE HYDROCHLORIDE:Hypersensitivity:0.23), (DICLOFENAC DIETHYLAMINE:Hypersensitivity:0.22), (DIENOGEST:Hypersensitivity:0.6), (DIETARY SUPPLEMENTFLAVOCOXID:Hypersensitivity:0.25), (DIETARY SUPPLEMENTHERBALSMILK THISTLE:Hypersensitivity:0.83), (DIETARY SUPPLEMENTRESVERATROL:Hypersensitivity:0.46), (DIHYDROCODEINE PHOSPHATE:Hypersensitivity:0.23), (DIMETHYL SULFOXIDE:Hypersensitivity:0.74), (DOBUTAMINEDOBUTAMINE HYDROCHLORIDE:Hypersensitivity:0.95), (DOCONEXENT ICOSAPENT FISH OIL OMEGA-3 FATTY ACIDS:Hypersensitivity:0.08), (DOCUSATE SODIUMSENNOSIDE ASENNOSIDE B:Hypersensitivity:0.26), (DOXERCALCIFEROL:Hypersensitivity:0.04), (DOXYLAMINE SUCCINATEPYRIDOXINE HYDROCHLORIDE:Hypersensitivity:0.68), (DROSPIRENONEESTRADIOL:Hypersensitivity: 0.27), (DYDROGESTERONEESTRADIOL:Hypersensitivity:0.41), (EDOXABAN TOSYLATE:Hypersensitivity:1.98), (ELOSULFASE ALFA:Hypersensitivity:0.64), (ELOTUZUMAB:Hypersensitivity:2.05), (EMPAGLIFLOZINME TFORMIN HYDROCHLORIDE:Hypersensitivity:0.26), (EMTRICITABINETENOFOVIR ALAFENAMIDE FUMARATE:Hypersensitivity:0.13), (ENALAPRIL MALEATELERCANIDIPINE HYDROCHLORIDE:Hypersensitivity:0.19), (EPHEDRINE HYDROCHLORIDEGUAIFENESIN:Hypersensitivity:1.42), (EPIRUBICIN HYDROCHLORIDE:Hypersensitivity:0.25), (ERGOLOID MESYLATES:Hypersensitivity:0.05), (ESTRADIOL BENZOATE:Hypersensitivity:1), (ESTROGENS, ESTERIFIED:Hypersensitivity:0.69), (ESTRONE:Hypersensitivity:0.19), (ETHINYL ESTRADIOLGESTODENE:Hypersensitivity:1.2), (ETRETINATE:Hypersensitivity:0.89), (EU CALYPTOLMENTHOLMETHYL SALICYLATETHYMOL:Hypersensitivity:0.5), (FENOPROFEN CALCIUM:Hypersensitivity:0.6), (FERRIC POLYSACCHARIDE COMPLEX:Hypersensitivity:0.67), (FERROUS BISGLYCINATE:Hypersensitivity:1.26), (FERROUS CITRATE:Hypersensitivity:0.12), (FERROUS SULFATEGLYCINE:Hypersensitivity:1.12), (FESOTERODINE:Hypersensitivity:0.2), (FIBRIN OGEN HUMAN:Hypersensitivity:0.57), (FLAVOXATE HYDROCHLORIDE:Hypersensitivity:0.53), (FLUCYTOSINE:Hypersensitivity:0.41), (FLUOR OURACILLEUCOVORIN CALCIUMOXALIPLATIN:Hypersensitivity:0.93), (FLUPENTIXOL DIHYDROCHLORIDEMELITRACEN HYDROCHLORIDE:Hypersensitivity:0.15), (FLUPHENAZINE HYDROCHLORIDE:Hypersensitivity:0.02), (FLUTAMIDE:Hypersensitivity:0.27), (FOLIC ACIDIRONMINERALSVITAMINS:Hypersensitivity:0.68), (FOSPHENYTOINFOSPHENY TOIN SODIUM:Hypersensitivity:0.19), (GARLIC OIL:Hypersensitivity:0.57), (GENTIAN VIOLET:Hypersensitivity:0.13), (GLUCONATE:Hypersensitivity:0.82), (GLYCOPYRROLATE INDACATEROL MALEATE:Hypersensitivity:1.63), (GLYCOSAMINOGLYCANS:Hypersensitivity:0.04), (GOL D:Hypersensitivity:0.21), (GREEN TEA LEAF:Hypersensitivity:0.45), (GUAIFENESINPHENYLEPHRINE HYDROCHLORIDE:Hypersensitivity:0.89), (HALCINONIDE:Hypersensitivity:0.7), (HALOPE RIDOL DECANOATE:Hypersensitivity:0.4), (HEMP:Hypersensitivity:0.12), (HERBALSHOMEOPAT HICSGOLDENSEAL:Hypersensitivity:0.73), (HERBALSVITIS VINIFERA SEED:Hypersensitivity:0.54), (HIGH DENSITY POLYETHYLENE:Hypersensitivity:0.54), (HORSE CHESTNUT:Hypersensitivity:1.33), (HYALURONIC ACIDLIDOCAINE HYDROCHLORIDE:Hypersensitivity:0.86), (HYALURONIDASE (HUMAN RECOMBINANT)RITUXIMAB:Hypersensitivity:0.4), (HYDROCHLORIC ACID:Hypersensitivity:0.25), (HYDROCHLOROTHIAZIDEMETOPROLOL TARTRATE:Hypersensitivity:0.14), (HYDROCHLOROTHIAZIDERAMIPRIL:Hypersensitivit y:0.25), (HYDROCODONEIBUPROFEN:Hypersensitivity:0.04), (IBRITUMOMAB TIUXETAN:Hypersensitivity:0.75), (IDARUBICIN:Hypersensitivity:0.45), (IMIPENEM:Hypers ensitivity:0.84), (IMIPRAMINE PAMOATE:Hypersensitivity:0.44), (INCOBOTULINUMTOXINA:Hypersensitivity:0.5), (INSU LIN PORKINSULIN PURIFIED PORK:Hypersensitivity:0.93), (INTERFERON:Hypersensitivity:2.65), (INTERFERON ALFA-2A:Hypersensitivity:1.64), (IODINEPOTASSIUM IODIDE:Hypersensitivity:0.63), (IOPAMIDOL:Hypersensitivity:1.08), (IRON POLYMALTOSE:Hypersensitivity:0.3), (IRONPOLYSACCHARIDES:Hypersensitivity:1.04), (ISOFLAVONES SOY:Hypersensitivity:0.35), (LAFUTIDINE:Hypersensitivity:0.03), (LANOLINMINERAL OILPETROLATUM:Hypersensitivity:0), (LATANOPROSTTIMOLOL:Hypersensitivity:0.48), (LETROZOLERIBOCICLIB:Hypersensitivity:0.12), (LEVAMLODIPINE:Hypersensitivity:0.11), (LEVAMLODIPINE BESYLATE:Hypersensitivity:0.45), (LEVOBUNOLOL:Hypersensitivity:0.07), (LEVOBUNOL OL HYDROCHLORIDE:Hypersensitivity:0.47), (LEVODOPA:Hypersensitivity:0.04), (LEVOLEUC OVORIN CALCIUM:Hypersensitivity:1.09), (LEVOMEFOLATE CALCIUMMETHYLCOBALAMINPYRIDOXAL 5-PHOSPHATE:Hypersensitivity:0.27), (LEVOMEFOLIC ACIDMETHYLCOBALAMINTURMERIC:Hypersensitivity:0.81), (LEVOTHYROXINE SODIUMPOTASSIUM IODIDE:Hypersensitivity:0.56), (LOMUSTINE:Hypersensitivity:0.35), (LUMACAFTOR:Hypers ensitivity:0.96), (LUTETIUM LU-177:Hypersensitivity:1.31), (LYCOPENE:Hypersensitivity:0.4), (MAGNESIUM GLUCEPTATE:Hypersensitivity:0.02), (MAGNESIUM PIDOLATE:Hypersensitivity:0.08), (MAPROTILINE HYDROCHLORIDE:Hypersensitivity:0.29), (MATRICARIA RECUTITA:Hypersensitivity:0.18), (MECHLORETHAMINE:Hypersensitivity:0.22), (MECLOZ INE DIHYDROCHLORIDE:Hypersensitivity:0.47), (MENINGOCOCCAL POLYSACCHARIDE VACCINE ANEISSERIA MENINGITIDIS GROUP A CAPSULAR POLYSACCHARIDE ANTIGEN:Hypersensitivity:0.11), (METFORMIN HYDROCHLORIDESAXAGLIPTIN:Hypersensitivity:0.44), (METFORMIN HYDROCHLORIDEVILDAGLIPTIN:Hypersensitivity:1.33), (METFORMIN PAMOATE:Hypersensitivity:0.06), (METHACHOLINE CHLORIDE:Hypersensitivity:0.01), (METHENAMINE MANDELATE:Hypersensitivity:1.24), (METHENOLONE ENANTHATE:Hypersensitivity:0.61), (METHYLPREDNISOLONE OR METHYLPREDNISOLONE ACETATE:Hypersensitivity:0.12), (MIGLUSTAT:Hypersensitivity:1.25), (MORNIFLUMATE: Hypersensitivity:0.34), (NABILONE:Hypersensitivity:0.36), (NADROPARIN CALCIUM:Hypersensitivity:1.76), (NALOXONE HYDROCHLORIDEPENTAZOCINE HYDROCHLORIDE:Hypersensitivity:0.12), (NEISSERIA MENINGITIDIS GROUP A CAPSULAR OLIGOSACCHARIDE DIPHTHERIA CRM197 PROTEIN CONJUGATE ANTIGENNEISSERIA MENINGITIDIS GROUP C CAPSULAR OLIGOSACCHARIDE DIPHTHERIA CRM197 PROTEIN CONJUGATE ANTIGENNEISSERIA MENINGITIDIS GROUP W-135 CAPSULAR OLIGOSACCHARIDE DIPHTHERIA CRM197 PROTEIN CONJUGATE ANTI:Hypersensitivity:0.05), (NICARDIPINE HYDROCHLORIDE:Hypersensitivity:0.43), (NOREPINEPHRINE:Hypersensitivity:1.04), (NUS INERSEN:Hypersensitivity:1.22), (NYSTATINTRIAMCINOLONE:Hypersensitivity:0.03), (OB ETICHOLIC ACID:Hypersensitivity:0.29), (OMEGA-6 FATTY ACIDS:Hypersensitivity:0.23), (ORLISTAT:Hypersensitivity:0.9), (ORNITHINE ASPARTATE:Hypersensitivity:0.16), (OXYQUINOLINE SULFATESODIUM LAURYL SULFATE:Hypersensitivity:0.18), (PAPAYA:Hypersensitivity:1.11), (PARSLEY:Hypersensitivit y:0.35), (PASSIFLORA INCARNATA FLOWER:Hypersensitivity:0.04), (PEGINTERFERON ALFA-2A OR PEGINTERFERON ALFA-2B:Hypersensitivity:0.07), (PENICILLIN V BENZATHINE:Hypersensitivity:0.01), (PENTOSTATIN:Hypersensitivity:1.14), (PERAMPANE L:Hypersensitivity:0.5), (PERINDOPRIL ARGININE:Hypersensitivity:0.26), (PERIPLANETA AMERICANA:Hypersensitivity:0.83), (PHENYLEPHRINE HYDROCHLORIDEWITCH HAZEL:Hypersensitivity:0.24), (PINUS STROBUS POLLEN:Hypersensitivity:0.36), (PLERIXAFOR:Hypersensitivity:0.44), (POLYETHYLENE GLYCOL 400:Hypersensitivity:0.58), (POLYMYXIN B SULFATETRIMETHOPRIM SULFATE:Hypersensitivity:0.34), (POTASSIUM BITARTRATE:Hypersensitivity:0.41), (POVIDONE:Hypersensitivity:0.77), (PREDNISOLONE ACETATESULFACETAMIDE SODIUM:Hypersensitivity:0.82), (PROCARBAZINE HYDROCHLORIDE:Hypersensitivity:0.07), (PROPARACAINE:Hypersensitivity:0.18), (PROT EIN:Hypersensitivity:0.13), (PROTRIPTYLINE HYDROCHLORIDE:Hypersensitivity:2.43), (PRUCALOPRIDE SUCCINATE:Hypersensitivity:0.6), (PSORALEN:Hypersensitivity:0.37), (QUERCUS RUBRA POLLEN:Hypersensitivity:0.38), (QUINIDINE GLUCONATE:Hypersensitivity:0.44), (RACEPINEPHRINE HYDROCHLORIDE:Hypersensitivity:0.01), (RED CLOVER:Hypersensitivity:0.35), (RED YEAST:Hypersensitivity:0.03), (REGORAFENIB:Hypersensitivity:2.07), (RESLIZUMAB:Hype rsensitivity:0.99), (RHUBARB:Hypersensitivity:0.3), (RILPIVIRINE HYDROCHLORIDE:Hypersensitivity:0.57), (SARILUMAB:Hypersensitivity:0.05), (SARPOGR ELATE:Hypersensitivity:0.27), (SELEGILINE HYDROCHLORIDE:Hypersensitivity:0.12), (SERRAPEPTASE:Hypersensitivity:0.98), (SODIU M CITRATE:Hypersensitivity:0.62), (SODIUM FLUORIDEVITAMINS:Hypersensitivity:0.46), (SODIUM PHOSPHATE, DIBASIC, ANHYDROUSSODIUM PHOSPHATE, DIBASIC, HEPTAHYDRATESODIUM PHOSPHATE, MONOBASIC, MONOHYDRATE:Hypersensitivity:0.76), (SODIUM PHOSPHATE, MONOBASIC, ANHYDROUS:Hypersensitivity:0), (STEMPHYLIUM SOLANI:Hypersensitivity:0.97), (SUFENTANIL CITRATE:Hypersensitivity:0.69), (TAZAROTENE:Hypersensitivity:0.58), (TEGASEROD:Hype rsensitivity:0.21), (TETANUS ANTITOXIN:Hypersensitivity:0.05), (TETRACAINE HYDROCHLORIDE:Hypersensitivity:0.1), (THEANINE:Hypersensitivity:0.2), (THYMOL:Hyp ersensitivity:0.11), (TIBOLONE:Hypersensitivity:1.38), (TILIDINE:Hypersensitivity:0.36), (TIM OLOL MALEATETRAVOPROST:Hypersensitivity:1.25), (TIMOLOLTRAVOPROST:Hypersensitivi ty:0.62), (TINIDAZOLE:Hypersensitivity:0.46), (TINZAPARIN:Hypersensitivity:1.53), (TIOPR ONIN:Hypersensitivity:0.64), (TOLMETIN:Hypersensitivity:0.15), (TOLMETIN SODIUM:Hypersensitivity:0.16), (TOLVAPTAN:Hypersensitivity:0.05), (TRAMETINIB DIMETHYL SULFOXIDE:Hypersensitivity:0.88), (TRANYLCYPROMINE:Hypersensitivity:0.14), (TRANY LCYPROMINE SULFATE:Hypersensitivity:0.74), (TRICHLORMETHIAZIDE:Hypersensitivity:0.34), (TRIENT INE HYDROCHLORIDE:Hypersensitivity:0.08), (TRIFLUOPERAZINE HYDROCHLORIDE:Hypersensitivity:0.09), (TRIPTORELIN PAMOATE:Hypersensitivity:1.27), (TYROSINE:Hypersensitivity:0.16), (VALRUBICIN:Hypers ensitivity:0.32), (VINORELBINEVINORELBINE TARTRATE:Hypersensitivity:0.73), (VITAMIN A PALMITATE:Hypersensitivity:0.76), (VOGLIBOSE:Hypersensitivity:0.17), (WARFARIN POTASSIUM:Hypersensitivity:0.25), (AMLODIPINE BESYLATEHYDROCHLOROTHIAZIDEOLMESARTAN MEDOXOMIL:Hypersomnia:0.2), (BENAZEPRIL HYDROCHLORIDE:Hypersomnia:0.16), (BIMATOPROST:Hypersomnia:0.25), (BRIVARACE TAM:Hypersomnia:0.02), (BUMETANIDE:Hypersomnia:0.24), (BUPRENORPHINE HYDROCHLORIDE:Hypersomnia:0.67), (CALCITRIOL:Hypersomnia:0.34), (CALCIUM CARBONATEVITAMIN D:Hypersomnia:0.22), (CALCIUMMAGNESIUMZINC:Hypersomnia:0.01), (CANNABIS SATIVA SUBSP. INDICA TOP:Hypersomnia:0.15), (CARVEDILOL PHOSPHATE:Hypersomnia:0.51), (CERTOLIZUMAB PEGOL:Hypersomnia:0.88), (CHLORTHALIDONE:Hypersomnia:0.19), (CIMETIDINE:Hypers omnia:0.14), (CLINDAMYCIN:Hypersomnia:0.34), (CLOTRIMAZOLE:Hypersomnia:0.09), (COD LIVER OIL:Hypersomnia:0.05), (CODEINE:Hypersomnia:0.27), (CORTICOTROPIN:Hypersomnia:0.01), (CORTISONEHYDROCORTISONE:Hypersomnia:0.17), (DACLIZUMAB:Hypersomnia:0.37), (DASATINIB:Hypersomnia:0.18), (DESLORATADINE:Hypersomnia:0.18), (DIGOXIN:Hyper somnia:0.54), (DIMENHYDRINATE:Hypersomnia:0.06), (DIPHENHYDRAMINE:Hypersomni a:0.77), (DOCUSATE:Hypersomnia:0.32), (DUTASTERIDE:Hypersomnia:0.44), (ENALAPRIL MALEATE:Hypersomnia:0.64), (ENOXAPARIN:Hypersomnia:0.34), (ERYTHROMYCIN:Hyp ersomnia:0.09), (ESTRADIOLNORETHINDRONE ACETATE:Hypersomnia:0.07), (ESTROGENS:Hypersomnia:0.18), (ESTRONESUS SCROFA OVARY:Hypersomnia:0.31), (ETHINYL ESTRADIOLNORGESTREL:Hypersomnia:0.12), (EVOLOCUMAB:Hypersomnia:0.83), (FEX OFENADINEPSEUDOEPHEDRINE:Hypersomnia:0.1), (FLUTICASONE:Hypersomnia:0.42), (FLUTICASONE FUROATE:Hypersomnia:0.13), (GUANFACINE:Hypersomnia:0.49), (HALOPERIDOL:Hypers omnia:0.64), (HERBALSTURMERIC:Hypersomnia:0.21), (HYDROCODONE BITARTRATEIBUPROFEN:Hypersomnia:0.08), (IBRUTINIB:Hypersomnia:0.28), (INSULIN DETEMIR:Hypersomnia:0.11), (ISOSORBIDE:Hypersomnia:0.18), (LABETALOLLABETAL OL HYDROCHLORIDE:Hypersomnia:0.46), (LACTASE:Hypersomnia:0.22), (LINAGLIPTIN:Hyp ersomnia:0.02), (METHYLPREDNISOLONE ACETATE:Hypersomnia:0.7), (MOXIFLOXACIN HYDROCHLORIDE:Hypersomnia:0.58), (NORETHINDRONE:Hypersomnia:0.41), (OSELTA MIVIR PHOSPHATE:Hypersomnia:0.03), (PANTOPRAZOLE:Hypersomnia:1.27), (PANTOPRAZOLE MAGNESIUM:Hypersomnia:0.45), (PEGINTERFERON BETA-1A:Hypersomnia:0.33), (PRAVASTATIN:Hypersomnia:1.33), (PRAZOSIN:Hypersomnia:0.37), (PROTRIPTYLINE HYDROCHLORIDE:Hypersomnia:0.07), (RABEPRAZOLE:Hypersomnia:0.05), (RANOLAZIN E:Hypersomnia:0.87), (RISEDRONATE SODIUM:Hypersomnia:0.4), (ROFLUMILAST:Hypersomnia:0.07), (SECUKINUMAB:Hyperso mnia:0.36), (SOTALOL:Hypersomnia:0.23), (SULFAMETHOXAZOLE:Hypersomnia:0.07), (TE LMISARTAN:Hypersomnia:0.26), (TERIFLUNOMIDE:Hypersomnia:0.75), (TIMOLOL:Hypers omnia:0.13), (TOFACITINIB CITRATE:Hypersomnia:0.09), (TORSEMIDE:Hypersomnia:0.11), (TRIAMTERENE:Hypersom nia:0.2), (UMECLIDINIUM BROMIDEVILANTEROL TRIFENATATE:Hypersomnia:0.84), (VITAMIN A:Hypersomnia:0.13), (.ALPHA.-TOCOPHEROLASCORBIC ACIDCALCIUM CITRATECHOLECALCIFEROLCOPPERDOCONEXENTDOCUSATE SODIUMFOLIC ACIDICOSAPENTIODINEIRONNIACINAMIDEPYRIDOXINE HYDROCHLORIDERIBOFLAVINTHIAMINEZINC:Hypertension:0.11), (.BETA.-HYDROXYISOVALERIC ACIDARGININEGLUTAMINE:Hypertension:1.34), (10-HYDROXYCAMPTOTHECIN:Hypertension:0.41), (3-METHOXYPHENCYCLIDINE:Hypertension:0.41), (4-HYDROXYBUTANOIC ACID:Hypertension:0.75), (6-ACETYLMORPHINE:Hypertension:0.2), (ABACAVIRLAMIVUDINEZIDOVUDINE:Hypert ension:0.82), (ACEPROMAZINE:Hypertension:1), (ACEPROMAZINE MALEATEACEPROMETAZINECLORAZEPATE DIPOTASSIUM:Hypertension:0.54), (ACETAMINOPHEN AND OR DEXTROMETHORPHAN AND OR DIPHENHYDRAMINE AND OR GUAIFENESIN AND OR PHENYLEPHRINE AND OR PSEUDOEPHEDRINE:Hypertension:1), (ACETAMINOPHENAMOBARBITALCAFFEINE CODEINE PHOSPHATE:Hypertension:0.97), (ACETAMINOPHENASCORBIC ACIDPHENIRAMINE MALEATE:Hypertension:0.7), (ACETAMINOPHENASCORBIC ACIDPHENYLEPHRINE HYDROCHLORIDESODIUM CITRATE:Hypertension:0.61), (ACETAMINOPHENASPIRINCAFFEINESALICYLAMIDE: Hypertension:0.78), (ACETAMINOPHENASPIRINDIPHENHYDRAMINE CITRATE:Hypertension:0.44), (ACETAMINOPHENASPIRINSALICYLAMIDE:Hypertensio n:0.99), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Hypertension:1.29), (ACETAMINOPHENBUCLIZINE HYDROCHLORIDECODEINE:Hypertension:0.93), (ACETAMINOPHENBUTABARBITAL CAFFEINE:Hypertension:0.77), (ACETAMINOPHENBUTYLSCOPOLAMINE BROMIDE:Hypertension:1.25), (ACETAMINOPHENCAFFEINEPHENYLEPHRINE HYDROCHLORIDE:Hypertension:0.55), (ACETAMINOPHENCAFFEINEPROPOXYPHEN E:Hypertension:0.75), (ACETAMINOPHENCAFFEINEPYRILAMINE MALEATE:Hypertension:0.51), (ACETAMINOPHENCHLORPHENIRAMINE MALEATEDEXTROMETHORPHAN HYDROBROMIDE:Hypertension:0.71), (ACETAMINOPHENCHLORPHENIRAMINE MALEATEDEXTROMETHORPHAN HYDROBROMIDEDOXYLAMINE SUCCINATEPHENYLEPHRINE HYDROCHLORIDE:Hypertension:0.66), (ACETAMINOPHENCHLORPHENIRAMINE MALEATEDEXTROMETHORPHAN HYDROBROMIDEPHENYLEPHRINE HYDROCHLORIDE OR ACETAMINOPHENDEXTROMETHORPHAN HYDROBROMIDEDOXYLAMINE SUCCINATEPHENYLEPHRINE HYDROCHLORIDE OR HOMEOPATHICS:Hypertension:0.9), (ACETAMINOPHENCHLORPHENIRAMINEDEXTR OMETHORPHANPHENYLEPHRINE:Hypertension:0.45), (ACETAMINOPHENCHLORZO XAZONE:Hypertension:1.19), (ACETAMINOPHENCODEINEPHENYLEPHRINE:Hypertens ion:0.27), (ACETAMINOPHENDEXTROMETHORPHAN HYDROBROMIDEDOXYLAMINE SUCCINATEPHENYLEPHRINE:Hypertension:0.43), (ACETAMINOPHENDEXTROMETH ORPHANDOXYLAMINEPSEUDOEPHEDRINE:Hypertension:0.84), (ACETAMINOPHEN DICHLORALPHENAZONEISOMETHEPTENE MUCATE:Hypertension:0.56), (ACETAMINOPHENDIPHENHYDRAMINE CITRATE:Hypertension:0.66), (ACETAMINOPHENDIPHENHYDRAMINE HYDROCHLORIDEPHENYLEPHRINE HYDROCHLORIDE:Hypertension:0.65), (ACETAMINOPHENDIPHENHYDRAMINE HYDROCHLORIDEPSEUDOEPHEDRINE HYDROCHLORIDE:Hypertension:0.62), (ACETAMINOPHENPHENYLEPHRINE:Hypertensi on:0.93), (ACETAMINOPHENPSEUDOEPHEDRINE:Hypertension:0.91), (ACETAMINOPHE NPSEUDOEPHEDRINE HYDROCHLORIDE:Hypertension:1.29), (ACETAMINOPHENPSEUDOEPHEDRINE HYDROCHLORIDETRIPROLIDINE HYDROCHLORIDE:Hypertension:0.41), (ACETIC ACIDCALCIUM CHLORIDEDEXTROSEMAGNESIUM CHLORIDEPOTASSIUM CHLORIDESODIUM CHLORIDE:Hypertension:0.72), (ACETOHYDROXAMIC ACID:Hypertension:1.16), (ACETONE:Hypertension:0.73), (ACETYLCHOLINE CHLORIDE:Hypertension:0.88), (ACLARUBICIN:Hypertension:0.34), (AGALSIDASE ALFA:Hypertension:0.83), (ALANINEAMINO ACIDSARGININECALCIUM CHLORIDEDEXTROSEGLYCINEHISTIDINEISOLEUCINELEUCINELYSINEMAGNE SIUM CHLORIDEMETHIONINEPHENYLALANINEPOTASSIUM PHOSPHATE, DIBASICPROLINEPROLINE, DL-SERINESODIUM ACETATESODIUM CHLORIDETHREONINETRYPTOPHANTYROSINEVALINE:Hypertension:0.24), (ALANI NEARGININEASPARTIC ACIDCYSTEINE HYDROCHLORIDEGLUTAMIC ACIDGLYCINEHISTIDINEISOLEUCINELEUCINELYSINE ACETATEMETHIONINEN-ACETYL-TYROSINEPHENYLALANINEPROLINESERINETAURINETHREONINETRYPTOPHA NTYROSINEVALINE:Hypertension:0.44), (ALANINEARGININEGLYCINEHISTIDINEI SOLEUCINELEUCINELYSINE HYDROCHLORIDEMETHIONINEPHENYLALANINEPROLINESERINETHREONINE TRYPTOPHANTYROSINEVALINE:Hypertension:0.43), (ALDESLEUKIN:Hypertension:1.21), (ALEFACEPT:Hypertension:0.67), (ALISERTIB:Hypertension:0.85), (ALISPORIVIR:Hypert ension:1.13), (ALITRETINOIN:Hypertension:0.47), (ALLANTOINBENZOCAINECAMPHOR (NATURAL)DIMETHICONEMENTHOL:Hypertension:1.23), (ALLERGENIC EXTRACT-CANDIDA:Hypertension:0.11), (ALMOTRIPTAN:Hypertension:0.84), (ALOSETRON:Hyperten sion:0.95), (ALPHA-TOCOPHEROL ACETATEASCORBIC ACIDBIOTINCHOLECALCIFEROLCYANOCOBALAMINDEXPANTHENOLFOLIC ACIDNIACINAMIDEPHYTONADIONEPYRIDOXINE HYDROCHLORIDERIBOFLAVIN 5′-PHOSPHATE SODIUMTHIAMINE HYDROCHLORIDEVITAMIN A PALMITATE:Hypertension:0.07), (ALPHA-TOCOPHEROLCHOLECALCIFEROL:Hypertension:1), (ALUMINUM ZIRCONIUM TETRACHLOROHYDREX GLY:Hypertension:0.47), (ALVIMOPAN:Hypertension:0.58), (ALVOCIDIB:Hypertension:0.62), (AMINOLEVULINIC ACID:Hypertension:0.3), (AMINOSALICYLATE SODIUM:Hypertension:0.7), (AMITRIPTYLINE HYDROCHLORIDECHLORDIAZEPOXIDE HYDROCHLORIDE:Hypertension:0.65), (AMITRIPTYLINEPERPHENAZINE:Hypertension: 0.92), (AMMONIA:Hypertension:0.57), (AMOBARBITAL SODIUM:Hypertension:1.27), (AMOROLFINE HYDROCHLORIDE:Hypertension:0.68), (AMPHETAMINE ADIPATEDEXTROAMPHETAMINE:Hypertension:0.21), (AMPHETAMINE ASPARTATE MONOHYDRATEAMPHETAMINE SULFATEDEXTROAMPHETAMINE SACCHARATEDEXTROAMPHETAMINE SULFATE:Hypertension:0.79), (AMPHETAMINEDEXTROAMPHETAMINE:Hypertension:0.85), (AMRUBICIN:Hypertension:0.82), (AMYLASE:Hypertension:1.16), (ANHYDROUS CITRIC ACIDBICARBONATE IONMAGNESIUM CATION:Hypertension:0.5), (ANHYDROUS CITRIC ACIDPOTASSIUM BICARBONATESODIUM BICARBONATE:Hypertension:0.54), (ANHYDROUS DEXTROSECALCIUM CHLORIDELACTIC ACIDMAGNESIUM CHLORIDEPOTASSIUM CHLORIDESODIUM BICARBONATESODIUM CHLORIDE:Hypertension:0.41), (ANHYDROUS DEXTROSELACTIC ACIDMAGNESIUM CHLORIDEPOTASSIUM CHLORIDESODIUM BICARBONATESODIUM CHLORIDE:Hypertension:0.38), (ANTHOXANTHUM ODORATUM POLLENDACTYLIS GLOMERATA POLLENLOLIUM PERENNE POLLENPHLEUM PRATENSE POLLENPOA PRATENSIS POLLEN:Hypertension:0.1), (ANTIHEMOPHILIC FACTOR HUMANFIBRINOGEN HUMANFACTOR XIII CONCENTRATE (HUMAN)VON WILLEBRAND FACTOR HUMAN:Hypertension:0.28), (ANTIHEMOPHILIC FACTOR, HUMAN RECOMBINANT RESIDUES 743-1636 DELETED:Hypertension:0.42), (ANTIPYRINEBENZOCAINE:Hypertension:0.74), (APATINI B:Hypertension:0.6), (APIS MELLIFERA VENOM:Hypertension:0.55), (APROTININ:Hypertension:0.5), (ARABICA COFFEE BEANATROPA BELLADONNACALCIUM PHOSPHATECHAMOMILE:Hypertension:0.56), (ARSENIC:Hypertension:0.47), (ARTESUN ATE:Hypertension:0.91), (ARTICAINE HYDROCHLORIDEEPINEPHRINE HYDROCHLORIDE:Hypertension:0.35), (ASCORBIC ACIDCYANOCOBALAMINFOLIC ACIDIRON:Hypertension:0.96), (ASCORBIC ACIDERGOCALCIFEROLRETINOLTOCOPHEROL:Hypertension:0.41), (ASCORBIC ACIDFERROUS FUMARATEFOLIC ACID:Hypertension:1.08), (ASCORBIC ACIDNIACINAMIDEPYRIDOXINE HYDROCHLORIDERIBOFLAVINTHIAMINE HYDROCHLORIDE:Hypertension:0.4), (ASCORBIC ACIDNIACINAMIDEPYRIDOXINERIBOFLAVINTHIAMINE:Hypertension:0.71), (ASPIR INCAFFEINEORPHENADRINE CITRATE:Hypertension:1.05), (ASPIRINCAFFEINEPROMETHAZINESALICYLAMIDE:H ypertension:1.05), (ASPIRINCAFFEINESALICYLAMIDE:Hypertension:1.05), (ASPIRINCA LCIUM CARBONATE:Hypertension:0.49), (ASPIRINCARISOPRODOL:Hypertension:0.55), (ASPIRI NCHLORPHENIRAMINE MALEATEPHENYLEPHRINE BITARTRATE:Hypertension:0.94), (ASPIRINDEXTROMETHORPHAN HYDROBROMIDEDOXYLAMINE SUCCINATEPHENYLEPHRINE BITARTRATE:Hypertension:0.85), (ASPIRINDIPHENHYDRAMINE:Hypertension:0.49), (AT AZANAVIR SULFATERITONAVIR:Hypertension:0.24), (ATLANTIC SALMON OIL:Hypertension:0.91), (ATOVAQUONEPROGUANIL:Hypertension:0.86), (ATROPA BELLADONNACALCIUM PHOSPHATECHAMOMILECOFFEA ARABICA FRUIT:Hypertension:0.41), (AURANOFIN:Hypertension:0.88), (AUTOLOGOUS CULTURED CHONDROCYTES:Hypertension:0.26), (AUTOLOGOUS CULTURED CHONDROCYTESSUS SCROFA COLLAGEN:Hypertension:0.82), (AVOBENZONEDIHYDROXYACETONEDIMETHICON E COPOLYOLHEXYL LAURATEHOMOSALATEOCTISALATEOCTOCRYLENEOXYBENZONEPROPYLEN E GLYCOL:Hypertension:0.18), (AVOBENZONEECAMSULEOCTOCRYLENE:Hypertension: 0.71), (AVOBENZONEHOMOSALATEOCTINOXATEOCTISALATEOCTOCRYLENEO XYBENZONE:Hypertension:0.26), (AVOBENZONEHOMOSALATEOCTINOXATEOCTIS ALATEOXYBENZONE:Hypertension:0.27), (AVOBENZONEHOMOSALATEOCTISALAT EOXYBENZONE:Hypertension:0.07), (AVOBENZONEOCTINOXATEOCTISALATEOXY BENZONE:Hypertension:0.36), (AVOBENZONEOCTISALATEOCTOCRYLENE:Hypertensi on:0.38), (AVOBENZONEOCTOCRYLENEOXYBENZONE:Hypertension:0.25), (BACILLU S CALMETTE-GUERIN SUBSTRAIN CONNAUGHT LIVE ANTIGEN:Hypertension:0.47), (BACITRACINNEOMYCIN SULFATE:Hypertension:1.44), (BACITRACINNEOMYCIN SULFATEPOLYMYXIN B SULFATE:Hypertension:1.23), (BACLOFENCYCLOBENZAPRINEDICLOFENACGABAP ENTINTETRACAINE:Hypertension:1.28), (BALSAM PERU OILCASTOR OILTRYPSIN:Hypertension:1.36), (BALSAM PERUCASTOR OIL:Hypertension:0.67), (BALSAM PERUCASTOR OILTRYPSIN:Hypertension:0.96), (BEDAQUILINE FUMARATE:Hypertension:1), (BELINOSTAT:Hypertension:0.85), (BELLADONNAERGOTA MINE TARTRATEPHENOBARBITAL:Hypertension:0.81), (BENFLUOREX:Hypertension:1.04), (BE NOXINATE HYDROCHLORIDEFLUORESCEIN SODIUM:Hypertension:0.16), (BENPERIDOL:Hypertension:0.93), (BENZOCAINELIDOCAIN E HYDROCHLORIDE:Hypertension:1.03), (BENZOCAINEMENTHOL:Hypertension:0.54), (BE NZOYL PEROXIDEERYTHROMYCIN:Hypertension:0.38), (BENZOYLECGONINE:Hypertension:0.49), (BERACTANT:Hypertension:0.25), (BETAINE:Hypertension:1.04), (BEZLOTOXUMAB:Hy pertension:0.68), (BICARBONATE IONCALCIUM CATIONLACTIC ACIDMAGNESIUM CATIONSODIUM CHLORIDE:Hypertension:0.13), (BIOTINCALCIUM PANTOTHENATECHOLINE BITARTRATECYANOCOBALAMINFOLIC ACIDINOSITOLNIACINAMIDEPYRIDOXINERIBOFLAVINTHIAMINE:Hypertension:10.04), (BIPERIDEN LACTATE:Hypertension:1.06), (BISMUTH SUBCITRATE:Hypertension:0.56), (BISMUTH TRISALICYLATE:Hypertension:0.29), (BLESELUMAB:Hypertension:1.39), (BORIC ACIDSODIUM BORATE:Hypertension:1.04), (BOTULINUM TOXIN NOS:Hypertension:0.47), (BOTULISM ANTITOXIN:Hypertension:1.13), (BROMPHENIRAMINE:Hypertension:0.78), (BROMPHENI RAMINE MALEATEDEXTROMETHORPHAN HYDROBROMIDEPHENYLEPHRINE HYDROCHLORIDE:Hypertension:0.66), (BUCLIZINE HYDROCHLORIDE:Hypertension:0.63), (BUDESONIDEINDACATEROL MALEATE:Hypertension:1.14), (BUPIVACAINE HYDROCHLORIDEFENTANYL CITRATE:Hypertension:0.41), (BUPRANOLOL:Hypertension:0.52), (BUPROPION HYDROBROMIDE:Hypertension:1.21), (BUSERELIN ACETATE:Hypertension:0.58), (CABOTEGRAVIR:Hypertension:0.55), (CAFFEINE CITRATE:Hypertension:0.76), (CAFFEINEMAGNESIUM SALICYLATE:Hypertension:0.91), (CAJUPUT OILEUCALYPTUS OILPEPPERMINT OIL:Hypertension:1.49), (CALCIUM CARBONATEDIMETHICONE:Hypertension:0.92), (CALCIUM CHLORIDEDEXTROSE MONOHYDRATEHYDROCHLORIC ACIDMAGNESIUM CHLORIDESODIUM BICARBONATESODIUM CHLORIDESODIUM HYDROXIDESODIUM LACTATE:Hypertension:1.09), (CALCIUM CHLORIDEDEXTROSE MONOHYDRATEPOTASSIUM CHLORIDESODIUM CHLORIDESODIUM LACTATE:Hypertension:0.02), (CALCIUM CHLORIDEDEXTROSEMAGNESIUM CHLORIDEOXIGLUTATIONEPOTASSIUM CHLORIDESODIUM BICARBONATESODIUM CHLORIDESODIUM PHOSPHATESODIUM PHOSPHATE, DIBASIC:Hypertension:0.04), (CALCIUM CHLORIDEMAGNESIUM CHLORIDEPOTASSIUM CHLORIDESODIUM BICARBONATESODIUM CHLORIDESODIUM PHOSPHATE, DIBASIC:Hypertension:0.65), (CALCIUM CHLORIDEMAGNESIUM CHLORIDESODIUM CHLORIDESODIUM LACTATE:Hypertension:0.69), (CALCIUM CHLORIDEPOTASSIUM CHLORIDESODIUM LACTATE:Hypertension:0.19), (CALCIUM CLDEXTROSEMAGNESIUM CLSODIUM BICARSODIUM CLSODIUM LACT:Hypertension:0.94), (CALCIUM CLMAGNESIUM CLPOTASSIUM CLSODIUM BICARSODIUM CLSODIUM PHOSPHATE,DIB:Hypertension:0.93), (CALCIUM GLYCEROPHOSPHATE:Hypertension:1.3), (CALCIUM LACTATECALCIUM PHOSPHATECHOLECALCIFEROL:Hypertension:1.02), (CALCIUM PANTOTHENATECUPRIC SULFATECYANOCOBALAMINFERROUS ASPARTO GLYCINATEFERROUS FUMARATEFOLIC ACIDMAGNESIUM SULFATENIACINAMIDEPYRIDOXINE HYDROCHLORIDERIBOFLAVINSODIUM ASCORBATETHIAMINE MONONITRATEZINC SULFATE:Hypertension:1.02), (CALFACTANT:Hypertension:0.18), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:Hypertension:1.15), (CAMPHOR (SYNTHETIC)MENTHOLMETHYL SALICYLATE:Hypertension:0.85), (CAMPHORATED PHENOL:Hypertension:0.64), (CAMPTOTHECIN:Hypertension:0.76), (CANDIDA ALBICANS:Hypertension:0.42), (CAPECITABINEOXALIPLATIN:Hypertension:0.89), (CAP REOMYCIN:Hypertension:0.74), (CAPSICUM:Hypertension:1.16), (CAPSICUM OLEORESIN:Hypertension:1), (CARBOPLATINPACLITAXEL:Hypertension:1.1), (CARBOX YMETHYLCELLULOSE SODIUMGELATINPECTIN:Hypertension:1.03), (CARGLUMIC ACID:Hypertension:0.69), (CARRAGEENANTITANIUMZINC:Hypertension:0.86), (CEFAM ANDOLE:Hypertension:0.72), (CEFATRIZINE:Hypertension:0.65), (CEFOZOPRAN:Hypertens ion:0.2), (CEFPROZIL:Hypertension:1.45), (CEFTIZOXIME:Hypertension:0.81), (CEFTOBIPR OLE:Hypertension:0.6), (CELERY SEED:Hypertension:0.72), (CENTELLA ASIATICA:Hypertension:1.04), (CEP-9722:Hypertension:0.57), (CEPHALEXIN ANHYDROUS:Hypertension:0.78), (CEPHALEXIN HYDROCHLORIDE:Hypertension:0.61), (CEPHALOSPORIN C:Hypertension:0.96), (CEPHALOTHIN SODIUM:Hypertension:0.67), (CERIVASTATIN SODIUM:Hypertension:1.51), (CETALKONIUM CHLORIDECHOLINE SALICYLATEMENTHOL:Hypertension:0.57), (CHAMOMILE:Hypertension:1), (CHENODIO L:Hypertension:0.74), (CHLOPHEDIANOL HYDROCHLORIDEDEXBROMPHENIRAMINE MALEATEPHENYLEPHRINE HYDROCHLORIDE:Hypertension:0.88), (CHLORAMPHENICOL PALMITATE:Hypertension:0.79), (CHLORELLA VULGARIS:Hypertension:0.77), (CHLORHEXIDINE ACETATE:Hypertension:0.47), (CHLORHEXIDINE GLUCONATECHLOROCRESOLHEXAMIDINE DIISETHIONATE:Hypertension:0.62), (CHLORODEHYDROMETHYLTESTOSTERONE:Hy pertension:0.71), (CHLOROPROCAINE:Hypertension:0.07), (CHLORPHENIRAMINE MALEATEHYDROCODONE BITARTRATE:Hypertension:0.13), (CHLORPHENIRAMINE MALEATEHYDROCODONE BITARTRATEPSEUDOEPHEDRINE HYDROCHLORIDE:Hypertension:1), (CHLORPROMAZINE HYDROCHLORIDEPHENOBARBITALPROMETHAZINE HYDROCHLORIDE:Hypertension:1), (CHOLECALCIFEROLCYANOCOBALAMIN CO-57\FOLIC ACIDPYRIDOXINE HYDROCHLORIDE:Hypertension:0.57), (CHOLIC ACID:Hypertension:1.23), (CHOLINE BITARTRATE:Hypertension:0.88), (CHOLINE SALICYLATE:Hypertension:0.74), (CHOLINETRAZODONE HYDROCHLORIDE:Hypertension:1.28), (CHONDROITIN SULFATE (BOVINE)HYALURONATE SODIUM:Hypertension:0.49), (CHROMIC CATIONCUPRIC CATIONFERROUS CATIONFLUORIDE IONIODIDE IONMANGANESE CATION (2+)SODIUM SELENIDEZINC CATION:Hypertension:0.99), (CHROMIC CHLORIDECUPRIC CHLORIDEMANGANESE CHLORIDEZINC CHLORIDE:Hypertension:0.57), (CILASTATIN SODIUM:Hypertension:0.95), (CILENGITIDE:Hypertension:0.6), (CIPROFLOXACINFLUOCI NOLONE ACETONIDE:Hypertension:0.21), (CITRIC ACID MONOHYDRATEDEXTROSESODIUM CITRATESODIUM PHOSPHATE, MONOBASIC:Hypertension:0.75), (CITRIC ACID MONOHYDRATEMETHYLCELLULOSE:Hypertension:0.58), (CITRIC ACID MONOHYDRATESODIUM CITRATE:Hypertension:0.47), (CITRULLINE:Hypertension:0.74), (CIXUTUMUMAB:Hyperte nsion:0.77), (CLINDAMYCIN PALMITATE HYDROCHLORIDE:Hypertension:0.87), (CLINDAMYCIN PHOSPHATETRETINOIN:Hypertension:0.74), (CLIOQUINOL:Hypertension:0.79), (CLOBET ASONE BUTYRATENYSTATINOXYTETRACYCLINE CALCIUM:Hypertension:1.12), (CLOPENTHIXOL HYDROCHLORIDE:Hypertension:0.6), (CLOPIDOGREL HYDROBROMIDE:Hypertension:0.89), (CLOPREDNOL:Hypertension:0.96), (CLORAZEPIC ACID:Hypertension:0.7), (COAGULATION FACTOR IX RECOMBINANT HUMAN:Hypertension:0.16), (COAGULATION FACTOR VII HUMAN:Hypertension:0.35), (COAGULATION FACTOR VIIA (RECOMBINANT):Hypertension:0.23), (COCAINE HYDROCHLORIDE:Hypertension:0.59), (CODEINE PHOSPHATEIBUPROFEN:Hypertension:0.74), (COLISTIN A SODIUM METHANESULFONATE:Hypertension:0.69), (COLISTIN SULFATE:Hypertension:0.83), (COLISTIN SULFATEHYDROCORTISONE ACETATENEOMYCIN SULFATETHONZONIUM BROMIDE:Hypertension:0.64), (COPPERINTRAUTERINE DEVICE:Hypertension:0.62), (COTININE:Hypertension:0.26), (CREATININE:Hypertension:1.07), (CRYOPRECIPITATED ANTIHEMOPHILIC FACTOR (HUMAN):Hypertension:0.16), (CUPRIC SULFATE ANHYDROUS:Hypertension:0.45), (CURCUMINDIETARY SUPPLEMENT:Hypertension:0.57), (CYCLOPENTOLATE HYDROCHLORIDEPHENYLEPHRINE HYDROCHLORIDE:Hypertension:0.33), (CYCLOPHOSPHAMIDECYTARABINEDEXAM ETHASONEDOXORUBICINMETHOTREXATEVINCRISTINE:Hypertension:1.01), (CYCL OPHOSPHAMIDEDOXORUBICINPREDNISONEVINCRISTINE:Hypertension:0.47), (CYS TEAMINE HYDROCHLORIDE:Hypertension:0.8), (CYSTEINE:Hypertension:1.27), (CYSTINE:Hypertens ion:1.26), (CYTARABINE HYDROCHLORIDE:Hypertension:0.5), (CYTARABINE OCFOSFATE:Hypertension:1.13), (DABRAFENIBTRAMETINIB:Hypertension:0.36), (DACE TUZUMAB:Hypertension:0.6), (DALBAVANCIN HYDROCHLORIDE:Hypertension:0.36), (DALFOPRISTINQUINUPRISTIN:Hypertension:0.4), (DALOTUZUMAB:Hypertension:0.69), (DASABUVIROMBITASVIRPARITAPREVIRRIB AVIRINRITONAVIR:Hypertension:0.82), (DAUNORUBICIN CITRATE:Hypertension:0.51), (DEOXYRIBONUCLIEC ACID:Hypertension:0.91), (DESLORATADINEPSEUDOEPHEDRINE SULFATE:Hypertension:1.06), (DEUTETRABENAZINE:Hypertension:1.05), (DEXPANTHEN OLNIACINAMIDEPYRIDOXINE HYDROCHLORIDERIBOFLAVINTHIAMINE HYDROCHLORIDE:Hypertension:0.7), (DEXTRAN 70\GLYCERINHYPROMELLOSES:Hypertension:0.64), (DEXTRAN 70\POLYETHYLENE GLYCOL 400\POVIDONETETRAHYDROZOLINE HYDROCHLORIDE:Hypertension:0.19), (DEXTRANHYPROMELLOSES:Hypertension:0.74), (DEXTROMETHORPHAN HYDROBROMIDEGUAIFENESINPSEUDOEPHEDRINE HYDROCHLORIDE:Hypertension:1.18), (DEXTROSEFRUCTOSETRISODIUM CITRATE DIHYDRATE:Hypertension:0.57), (DEXTROSEHEPARIN:Hypertension:0.65), (DEXTROSE LIDOCAINE HYDROCHLORIDE:Hypertension:0.74), (DEXTROSESODIUM CHLORIDE:Hypertension:0.76), (DIBUCAINE:Hypertension:1.07), (DIBUCAINE HYDROCHLORIDEPREDNISOLONE:Hypertension:1.08), (DICHLORPHENAMIDE:Hyperte nsion:1.23), (DIENOGEST:Hypertension:1.28), (DIHYDROERGOTAMINE:Hypertension:0.82), (DILOXANIDE FUROATETINIDAZOLE:Hypertension:0.46), (DINUTUXIMAB:Hypertension:0.51), (DIPHEN HYDRAMINE HYDROCHLORIDEPHENYLEPHRINE HYDROCHLORIDE:Hypertension:1.05), (DIPHENHYDRAMINE HYDROCHLORIDEZINC ACETATE:Hypertension:0.32), (DIPHTHERIA AND TETANUS TOXOIDS AND POLIOVIRUS VACCINE ANTIGENS:Hypertension:0.4), (DISODIUM FUMARATE:Hypertension:0.87), (DOCETAXEL ANHYDROUS:Hypertension:0.26), (DOCONEXENTICOSAPENTPHOSPHATIDYL SERINE:Hypertension:0.47), (DOCUSATE SODIUMSENNOSIDE ASENNOSIDE B:Hypertension:1.14), (DOXIFLURIDINE:Hypertension:0.76), (DOXYLAMINEPYRIDOXINE:Hypertension:0.27), (DROSPIRENONE:Hypertension:1), (DROTRECOGIN ALFA (ACTIVATED):Hypertension:0.47), (DUVELISIB:Hypertension:0.78), (DYCLONINE HYDROCHLORIDE:Hypertension:1), (EDETIC ACID:Hypertension:0.48), (EFALIZUMAB:Hypertension:1.09), (EFAVIRENZLAMIVUDINE ZIDOVUDINE:Hypertension:0.39), (EFLORNITHINE:Hypertension:0.71), (EFMOROCTOCOG ALFA:Hypertension:0.16), (ELECTROLYTES NOSMINERALSSORBITOL:Hypertension:0.37), (ELETRIPTAN:Hypertension:0.82), (ELM:H ypertension:0.7), (EMACTUZUMAB:Hypertension:0.87), (EMTRICITABINETENOFOVIRZI DOVUDINE:Hypertension:0.66), (EMU OIL:Hypertension:0.95), (ENASIDENIB MESYLATE:Hypertension:0.99), (ENBUCRILATE:Hypertension:0.38), (ENTINOSTAT:Hypert ension:0.78), (ENTOSPLETINIB:Hypertension:0.56), (EPHEDRINE HYDROCHLORIDEGUAIFENESIN:Hypertension:1.13), (EPHEDRINE SULFATE:Hypertension:0.83), (EPINEPHRINE HYDROCHLORIDE:Hypertension:1.19), (EPINEPHRINELIDOCAINE HYDROCHLORIDE:Hypertension:0.53), (EQUISETUM ARVENSE TOP:Hypertension:0.59), (ERGONOVINE MALEATE:Hypertension:0.74), (ERGONOVINEOXYTOCIN:Hypertension:0.18), (ERGOTA MINE TARTRATE:Hypertension:0.76), (ERYTHROMYCIN ESTOLATE:Hypertension:0.53), (ESCHERICHIA COLI:Hypertension:1.01), (ESSENTIAL OILS:Hypertension:0.99), (ESTRADIOL BENZOATE:Hypertension:1.34), (ESTRADIOL HEMIHYDRATENORETHINDRONE ACETATE:Hypertension:0.7), (ESTRADIOL VALERATENORETHINDRONE:Hypertension:0.71), (ESTRADIOLNOMEGESTROL ACETATE:Hypertension:0.44), (ESTRAMUSTINE PHOSPHATE:Hypertension:1.05), (ESTROGENS, CONJUGATEDNORGESTREL:Hypertension:0.64), (ESTROGENSPROGESTERONE:Hypert ension:0.45), (ESTRONE:Hypertension:0.99), (ETELCALCETIDE HYDROCHLORIDE:Hypertension:1.25), (ETEPLIRSEN:Hypertension:1.03), (ETHAMBUTOL HYDROCHLORIDEISONIAZIDPYRAZINAMIDERIFAMPIN:Hypertension:0.81), (ETHA MBUTOL HYDROCHLORIDEISONIAZIDPYRAZINAMIDERIFAMYCIN:Hypertension:0.74), (ETHI NYL ESTRADIOLETHYNODIOL:Hypertension:0.69), (ETHINYL ESTRADIOLLEVONORGESTREL ACETATE:Hypertension:0.91), (ETHOSUXIMIDE:Hypertension:0.3), (ETHOTOIN:Hypertensi on:0.1), (ETHYL CHLORIDE:Hypertension:0.49), (ETHYLMORPHINE:Hypertension:0.51), (ETHYLPHENIDA TE:Hypertension:0.82), (ETILEFRINE:Hypertension:0.3), (EZETIMIBEROSUVASTATIN:Hyp ertension:1.11), (FACTOR IX COMPLEX:Hypertension:0.8), (FARLETUZUMAB:Hypertension:0.57), (FECAL MICROBIOTA:Hypertension:0.29), (FENRETINIDE:Hypertension:0.84), (FENTANYL HYDROCHLORIDE:Hypertension:0.67), (FERROUS ASPARTO GLYCINATEFERROUS FUMARATE:Hypertension:0.34), (FERUMOXYTOL:Hypertension:1.16), (FEVERFEW:Hypert ension:0.79), (FILANESIB:Hypertension:0.9), (FLOXURIDINE:Hypertension:0.81), (FLUDEOX YGLUCOSE F-18:Hypertension:1.04), (FLUMETHASONE PIVALATE:Hypertension:1.21), (FLUOCORTOLONE:Hypertension:0.8), (FLUOROMETHOL ONE ACETATE:Hypertension:0.67), (FLUOROURACILIRINOTECANLEUCOVORINOXALIPL ATIN:Hypertension:0.65), (FLUPENTIXOL DIHYDROCHLORIDE:Hypertension:0.88), (FOSBRETABULIN TROMETHAMINE:Hypertension:1.31), (FRANGULA PURSHIANA BARK:Hypertension:0.95), (FROVATRIPTAN:Hypertension:0.8), (FRUCTOSE:Hypertension:0.72), (FURAZOLIDONE:Hypertension:0.84), (GADOFOSVESET TRISODIUM:Hypertension:0.11), (GADOTERIDOL:Hypertension:0.04), (GANITUMAB:Hyper tension:0.55), (GILTERITINIB:Hypertension:1), (GIMERACIL:Hypertension:0.65), (GINSENG EXTRACT:Hypertension:0.94), (GLUCARPIDASE:Hypertension:0.99), (GLUCONATE:Hypert ension:1.29), (GLYCERINHYPROMELLOSE 2910 (15 MPA.S)POLYETHYLENE GLYCOL 400\PROPYLENE GLYCOL:Hypertension:1.1), (GLYCERINHYPROMELLOSESPOLYETHYLENE GLYCOL 400\TETRAHYDROZOLINE HYDROCHLORIDE:Hypertension:0.24), (GLYCERINMEDIUM-CHAIN TRIGLYCERIDESPHOSPHOLIPIDSOYBEAN OIL:Hypertension:0.75), (GLYCEROL PHENYLBUTYRATE:Hypertension:0.46), (GOTU KOLA:Hypertension:0.72), (GRAPEFRUIT SEED EXTRACT:Hypertension:0.94), (HELIUM:Hypertension:0.57), (HEPARIN SODIUMSODIUM CHLORIDE:Hypertension:0.58), (HEPATITIS A VIRUS STRAIN CR 326F ANTIGEN (FORMALDEHYDE INACTIVATED):Hypertension:0.25), (HEPATITIS B VIRUS HBSAG SURFACE PROTEIN ANTIGEN:Hypertension:0.54), (HEPATITIS B VIRUS SUBTYPE ADW2 HBSAG SURFACE PROTEIN ANTIGEN:Hypertension:0.28), (HEPTAMINOL:Hypertension:1.02), (HERBALSHOMEOPAT HICSGOLDENSEAL:Hypertension:0.82), (HERBSUNSPECIFIED INGREDIENT:Hypertension:0.75), (HEXACHLOROPHENE:Hypertension:0.96), (HEXAMIDI NE DIISETHIONATE:Hypertension:0.56), (HEXAMINOLEVULINATE HYDROCHLORIDE:Hypertension:0.29), (HOMEOPATHICSZINC ACETATE ANHYDROUSZINC GLUCONATE:Hypertension:0.5), (HOMEOPATHICSZINC ACETATEZINC GLUCONATE:Hypertension:0.61), (HOMOSALATEOCTINOXATEOCTISALATEOXYBE NZONE:Hypertension:0.27), (HUMAN CLOSTRIDIUM TETANI TOXOID IMMUNE GLOBULIN:Hypertension:0.56), (HUMAN COAGULATION FACTOR VIII/VON WILLEBRAND FACTOR COMPLEX:Hypertension:0.28), (HUMAN COLOSTRUM:Hypertension:1.03), (HUMAN PAPILLOMAVIRUS QUADRIVALENT (TYPES 6, 11, 16, AND 18) VACCINE, RECOMBINANT:Hypertension:0.34), (HUMAN PAPILLOMAVIRUS TYPE 11 L1 CAPSID PROTEIN ANTIGENHUMAN PAPILLOMAVIRUS TYPE 16 L1 CAPSID PROTEIN ANTIGENHUMAN PAPILLOMAVIRUS TYPE 18 L1 CAPSID PROTEIN ANTIGENHUMAN PAPILLOMAVIRUS TYPE 31 L1 CAPSID PROTEIN ANTIGENHUMAN PAPILLOMAVIRUS TYPE 33 L1 CAPSID PROTEIN ANTIGENHUMAN PAPILLOMAVIRUS TYPE:Hypertension:0.29), (HUMAN ROTAVIRUS A TYPE GIP(8) STRAIN RIX4414 LIVE ANTIGEN:Hypertension:0.03), (HUMAN ROTAVIRUS A TYPE G1P7(5) STRAIN WI79 LIVE ANTIGENHUMAN ROTAVIRUS A TYPE G2P7(5) STRAIN SC2 LIVE ANTIGENHUMAN ROTAVIRUS A TYPE G3P7(5) STRAIN WI78 LIVE ANTIGENHUMAN ROTAVIRUS A TYPE G4P7(5) STRAIN BRB LIVE ANTIGENHUMAN ROTAVIRUS A TYPE G6P1A(8) STRAIN WI79 LIVE ANTIGEN:Hypertension:0.23), (HUMAN VARICELLA-ZOSTER IMMUNE GLOBULIN:Hypertension:0.81), (HYALURONIDASE:Hypertension:0.39), (HYALURONIDASE (HUMAN RECOMBINANT):Hypertension:0.36), (HYALURONIDASE RECOMBINANT HUMAN:Hypertension:0.97), (HYALURONIDASE, OVINE:Hypertension:0.21), (HYDROCODONEHYDROCODONE BITARTRATEIBUPROFEN:Hypertension:0.71), (HYDROCORTISONE PROBUTATE:Hypertension:1.21), (HYDROCORTISONEPRAMOXINE:Hypertension:0.71), (HYOSCYAMINE SULFATEMETHENAMINEMETHYLENE BLUESODIUM PHOSPHATE, MONOBASIC:Hypertension:0.81), (HYPOCHLORITE ION:Hypertension:0.52), (IBUPROFEN SODIUM:Hypertension:0.99), (IDEBENONE:Hypertension:0.9), (IFOSFAMIDEMESNA:Hyper tension:0.82), (ILOPROST TROMETHAMINE:Hypertension:0.68), (IMIPRAMINE PAMOATE:Hypertension:1.05), (INDIUM IN-111 CHLORIDEINDIUM IN-111 PENTETREOTIDEPENTETREOTIDE:Hypertension:0.62), (INFLUENZA A VIRUS A/CALIFORNIA/7/2009 (H1N1) RECOMBINANT HEMAGGLUTININ ANTIGENINFLUENZA A VIRUS A/VICTORIA/361/2011 (H3N2) RECOMBINANT HEMAGGLUTININ ANTIGENINFLUENZA B VIRUS B/WISCONSIN/1/2010 RECOMBINANT HEMAGGLUTININ ANTIGEN:Hypertension:0.4), (INFLUENZA A VIRUS A/CALIFORNIA/7/2009 X-179A (H1N1) ANTIGEN (FORMALDEHYDE INACTIVATED)INFLUENZA A VIRUS A/TEXAS/50/2012 X-223A (H3N2) ANTIGEN (FORMALDEHYDE INACTIVATED)INFLUENZA B VIRUS B/BRISBANE/60/2008 ANTIGEN (FORMALDEHYDE INACTIVATED)INFLUENZA B VIRUS B/MASSACHUSETTS/2/2012 ANTIGEN (FORMALDE:Hypertension:1.02), (INFLUENZA A VIRUS A/CALIFORNIA/7/2009 X-181 (H1N1) ANTIGEN (FORMALDEHYDE INACTIVATED)INFLUENZA A VIRUS A/VICTORIA/210/2009 X-187 (H3N2) ANTIGEN (FORMALDEHYDE INACTIVATED)INFLUENZA B VIRUS B/BRISBANE/60/2008 ANTIGEN (FORMALDEHYDE INACTIVATED)INFLUENZA VIRUS VACCINE:Hypertension:1.04), (INIPARIB:Hypertension:0.5), (INSULIN DEFALAN (PORCINE):Hypertension:0.83), (IOBENGUANE I-123:Hypertension:0.28), (IOBENGUANE I-131:Hypertension:0.36), (IODINE POVACRYLEXISOPROPYL ALCOHOL:Hypertension:0.39), (IODINETOSITUMOMAB:Hypertension:0.73), (IOTHALAM ATE MEGLUMINE:Hypertension:0.18), (IOTHALAMIC ACID:Hypertension:0.85), (IOXAGLATE MEGLUMINEIOXAGLATE SODIUM:Hypertension:0.72), (IRON ISOMALTOSIDE 1000:Hypertension:0.55), (ISATUXIMAB:Hypertension:0.73), (ISAVUCONAZONIUM:Hyperte nsion:0.61), (ISEPAMICIN:Hypertension:0.65), (ISOFLAVONES SOY:Hypertension:1.22), (ISOMETHEPTENE:Hypertension:0.75), (ISONIAZIDPYRIDOXINE:Hypertension:0.52), (ISONIAZIDPYRIDOXINE HYDROCHLORIDE:Hypertension:0.73), (ISOPROPYL ALCOHOLTENECTEPLASE:Hypertension:0.84), (ISOPROPYL MYRISTATE:Hypertension:0.86), (ISOPROTERENOL:Hypertension:0.92), (ISOSULFAN BLUE:Hypertension:0.44), (JWH-018:Hypertension:0.89), (KAOLIN:Hypertension:1.1), (KETAZOLAM:Hypertension:1.1), (KET OBEMIDONE:Hypertension:0.77), (LACTOFERRIN:Hypertension:0.46), (LAMIVUDINENEV IRAPINESTAVUDINE:Hypertension:0.45), (LAMIVUDINENEVIRAPINEZIDOVUDINE:H ypertension:0.02), (LAMIVUDINETENOFOVIR DISOPROXIL FUMARATE:Hypertension:0.01), (LANOLINMINERAL OIL:Hypertension:0.87), (LANOLINMINERAL OILPETROLATUM:Hypertension:1.29), (LAVENDER OIL:Hypertension:1.16), (LESTAURTINIB:Hypertension:0.86), (LETROZOLERIBOCICLIB:H ypertension:1.13), (LEUCINE:Hypertension:0.9), (LEUPROLIDE ACETATENORETHINDRONE ACETATE:Hypertension:0.1), (LEVAMISOLE:Hypertension:0.54), (LEVOBUPIVACAINE:Hy pertension:0.72), (LEVODOPAUNSPECIFIED INGREDIENT:Hypertension:0.71), (LEVOMEFOLIC ACIDMETHYLCOBALAMINTURMERIC:Hypertension:0.87), (LEVORPHANOL:Hypertens ion:0.94), (LEVOSIMENDAN:Hypertension:1.96), (LIDOCAINE HYDROCHLORIDEMETHYLPREDNISOLONE ACETATE:Hypertension:0.83), (LIDOCAINEPHENYLEPHRINE:Hypertension:0.2), (LINOLE IC ACID:Hypertension:0.91), (LIRILUMAB:Hypertension:0.69), (LITHIUM OROTATE:Hypertension:0.89), (LOBAPLATIN:Hypertension:0.2), (LORLATINIB:Hypertensio n:0.9), (LUMACAFTOR:Hypertension:1.07), (LYMPHOCYTES:Hypertension:0.17), (LYSERGI DE:Hypertension:0.3), (MAGNESIUM ACETATE:Hypertension:1.09), (MAGNESIUM ACETATE TETRAHYDRATEPOTASSIUM ACETATESODIUM CHLORIDE:Hypertension:0.67), (MAGNESIUM ALUMINUM SILICATE:Hypertension:0.87), (MAGNESIUM CHLORIDEPOTASSIUM CHLORIDEPOTASSIUM PHOSPHATESODIUM ACETATESODIUM CHLORIDESODIUM GLUCONATESODIUM PHOSPHATE, DIBASIC:Hypertension:0.67), (MAGNESIUM GLUCEPTATE:Hypertension:0.89), (MAGNESIUM PHOSPHATE, TRIBASIC, PENTAHYDRATE:Hypertension:0.85), (MAGNESIUM TRISILICATE:Hypertension:0.28), (MALIC ACID:Hypertension:0.84), (MAPROTILINE:Hypertension:1.18), (MARIZOMIB:Hypertension:1.1), (MATRICARIA RECUTITA:Hypertension:1), (MAZINDOL:Hypertension:0.83), (MEASLES VIRUS STRAIN ENDERS' ATTENUATED EDMONSTON LIVE ANTIGENMUMPS VIRUS STRAIN B LEVEL JERYL LYNN LIVE ANTIGENRUBELLA VIRUS STRAIN WISTAR RA 27/3 LIVE ANTIGENVARICELLA-ZOSTER VIRUS STRAIN OKA/MERCK LIVE ANTIGEN:Hypertension:0.01), (MECHLORETHAMINE OXIDE HYDROCHLORIDE:Hypertension:0.83), (MELITRACEN:Hypertension:0.53), (MEPHENESIN: Hypertension:0.67), (MEROPENEM ANHYDROUS:Hypertension:0.86), (METHACHOLINE CHLORIDE:Hypertension:0.58), (METHANDROSTENOLONE:Hypertension:0.64), (METHOT REXATEMETHOTREXATE SODIUM:Hypertension:0.59), (METHSCOPOLAMINEMETHSCOPOLAMINE BROMIDE:Hypertension:0.76), (METHSUXIMIDE:Hypertension:0.92), (METHYLPREDNISO LONE OR METHYLPREDNISOLONE ACETATE:Hypertension:1.34), (METHYLPREDNISOLONE SODIUM PHOSPHATE:Hypertension:0.98), (METHYSERGIDE MALEATE:Hypertension:0.92), (MEZLOCILLIN SODIUM:Hypertension:0.54), (MICONAZOLE NITRATE OR TERBINAFINE HYDROCHLORIDE OR TOLNAFTATE:Hypertension:0.64), (MIDOMAFETAMINE:Hypertension:0.52), (MIFAMURTI DE:Hypertension:1.01), (MILTEFOSINE:Hypertension:0.54), (MINERAL OIL EMULSIONPETROLATUM:Hypertension:0.69), (MITOGUAZONE:Hypertension:0.13), (MIT RAGYNINE:Hypertension:0.68), (MITRAGYNINEHERBALS:Hypertension:0.46), (MIVACU RIUM CHLORIDE:Hypertension:0.73), (MOGAMULIZUMAB:Hypertension:1.12), (MOLYBDENUM MO-99\TECHNETIUM TC-99M:Hypertension:0.05), (MONOSODIUM GLUTAMATE:Hypertension:0.67), (MOXIFLOXACINTRIAMCINOLONE:Hypertension:0.64), (MUROMONAB-CD3:Hypertension:0.2), (MYCOPHENOLATE MOFETIL HYDROCHLORIDE:Hypertension:0.94), (NALBUPHINE:Hypertension:1.41), (NALOXONE HYDROCHLORIDEPENTAZOCINE HYDROCHLORIDE:Hypertension:1), (NATURAL LATEX RUBBER:Hypertension:1.06), (NEOMYCIN SULFATEPOLYMYXIN B SULFATE:Hypertension:0.62), (NEOSTIGMINE BROMIDE:Hypertension:1.3), (NESIRITIDE:Hypertension:0.89), (NETILMICIN:Hypertension: 0.68), (NIACINAMIDEPYRIDOXINE HYDROCHLORIDERIBOFLAVINTHIAMINE HYDROCHLORIDE:Hypertension:0.96), (NIACINAMIDEPYRIDOXINERIBOFLAVINTHI AMINE:Hypertension:1.1), (NIMETAZEPAM:Hypertension:0.95), (NIMOTUZUMAB:Hyperten sion:0.59), (NORETHANDROLONE:Hypertension:1), (NORGESTIMATE:Hypertension:0.52), (NORGESTREL:Hypertension:0.45), (NUSINERSEN:Hypertension:0.78), (OCTENIDINEPHEN OXYETHANOL:Hypertension:0.42), (OCTINOXATEOCTISALATEZINC OXIDE:Hypertension:0.2), (OCTINOXATEOCTOCRYLENE:Hypertension:0.22), (OCTINOX ATEOXYBENZONE:Hypertension:0.53), (OCTINOXATETITANIUM DIOXIDEZINC OXIDE:Hypertension:0.29), (OCTOTIAMINE:Hypertension:1.32), (OK-432:Hypertension:1.34), (OLARATUMAB:Hypertension:0.62), (OLIVE OILSOYBEAN OIL:Hypertension:0.16), (OLSALAZINE:Hypertension:0.94), (OLSALAZINE SODIUM:Hypertension:0.48), (OMACETAXINE:Hypertension:1.18), (OPICAPONE:Hypertensi on:1.18), (OREGANO:Hypertension:0.81), (OTERACIL:Hypertension:0.54), (OTERACIL POTASSIUM:Hypertension:0.7), (OXITROPIUM BROMIDE:Hypertension:0.85), (OXYCODONE TEREPHTHALATE:Hypertension:0.69), (OXYMETAZOLINE HYDROCHLORIDEPSEUDOEPHEDRINE HYDROCHLORIDE:Hypertension:0.6), (OXYMETAZOLINE HYDROCHLORIDETETRACAINE HYDROCHLORIDE:Hypertension:0.56), (OXYQUINOLINE SULFATESODIUM LAURYL SULFATE:Hypertension:0.9), (PALIFERMIN:Hypertension:0.71), (PANCURONIUM:Hypertens ion:1.37), (PARALDEHYDE:Hypertension:0.72), (PAROMOMYCIN:Hypertension:0.79), (PAR OMOMYCIN SULFATE:Hypertension:0.87), (PARSLEY:Hypertension:1.21), (PASCOPYRUM SMITHII POLLEN:Hypertension:1.15), (PASSIFLORA INCARNATA FLOWER:Hypertension:0.81), (PEGADEMASE BOVINE:Hypertension:0.23), (PEGINTERFERON ALFA-2B\RIBAVIRIN:Hypertension:0.9), (PENBUTOLOL SULFATE:Hypertension:1.05), (PENICILLIN G PROCAINE:Hypertension:0.8), (PENICILLIN G SODIUM:Hypertension:0.58), (PENTACHLOROPHENOL:Hypertension:0.54), (PENTOBARBI TAL:Hypertension:1.1), (PENTOBARBITAL SODIUM:Hypertension:0.73), (PERAZINE:Hypertension:1.05), (PERGOLIDE:Hypertension:0.32), (PHENACETIN:Hypertension:0.83), (PHENETHICILLIN POTASSIUM:Hypertension:0.69), (PHENYLALANINE, DL-:Hypertension:0.7), (PHENYLEPHRINE HYDROCHLORIDEWITCH HAZEL:Hypertension:1.51), (PHOSPHAZIDE:Hypertension:0.36), (PHTHALYLSULFATHIAZ OLE:Hypertension:0.44), (PICOLAMINE SALICYLATE:Hypertension:1.07), (PIMAVANSERIN:Hypertension:0.74), (PIPER METHYSTICUM ROOT:Hypertension:0.77), (PIPOTIAZINE PALMITATE:Hypertension:0.81), (PLASMA PROTEIN FRACTION (HUMAN):Hypertension:1.43), (PLATELET CONCENTRATE HUMAN:Hypertension:0.1), (PLATINUM:Hypertension:1), (POLIOMYELITIS VACCINE:Hypertension:0.88), (POLOXAMER:Hypertension:1.01), (POLYETHYLENE GLYCOL 400\POLYVINYL ALCOHOL:Hypertension:0.24), (POLYLACTIDE, L-:Hypertension:0.36), (POLYMYXIN B SULFATETRIMETHOPRIM:Hypertension:0.15), (POTASSIUM BITARTRATE:Hypertension:1.05), (POTASSIUM BROMIDE:Hypertension:0.85), (POTASSIUM CITRATESODIUM CITRATEZINC:Hypertension:1.16), (POTASSIUM HYDROXIDEPOTASSIUM PHOSPHATE, DIBASICSODIUM PHOSPHATE, DIBASIC, ANHYDROUS:Hypertension:0.83), (PRACINOSTAT:Hypertension:0.78), (PRAZIQUANTEL: Hypertension:0.24), (PRETOMANID:Hypertension:0.49), (PROGUANIL:Hypertension:0.43), (P ROPARACAINE:Hypertension:0.24), (PROPOXYPHENE NAPSYLATE:Hypertension:0.79), (PROPYPHENAZONE:Hypertension:0.75), (PROSTATE:Hy pertension:0.94), (PROTEIN C:Hypertension:0.49), (PROTIONAMIDE:Hypertension:0.63), (PSORALEN:Hypertension:1.39), (PYRANTEL PAMOATE:Hypertension:0.56), (PYRIMETHAMINESULFADOXINE:Hypertension:0.53), (Q UINAGOLIDE HYDROCHLORIDE:Hypertension:0.91), (QUINIDINE GLUCONATE:Hypertension:1.2), (RABIES IMMUNE GLOBULIN (HUMAN):Hypertension:0.39), (RABIES VACCINE:Hypertension:0.32), (RACEPINEPHRINE HYDROCHLORIDE:Hypertension:0.69), (RADIUM:Hypertension:0.96), (RETAPAMULIN:Hy pertension:0.78), (RHAMNUS PURSHIANA BARK EXTRACT:Hypertension:0.51), (RIBOFLAVIN 5′-PHOSPHATE SODIUM:Hypertension:0.35), (RIBONUCLEIC ACID:Hypertension:0.8), (RIGOSERTIB:Hypertension:0.92), (RILOTUMUMAB:Hypertension: 0.97), (RINGER'S SOLUTION, LACTATED:Hypertension:0.43), (ROLAPITANT:Hypertension:0.96), (ROMOSOZUMAB:Hyp ertension:0.91), (RUBIDIUM CHLORIDE RB-82:Hypertension:0.41), (RUSCUS ACULEATUS ROOT:Hypertension:1.3), (RUXOLITINIB PHOSPHATE:Hypertension:1.06), (SACROSIDASE:Hypertension:0.67), (SAFINAMIDE:Hyper tension:1.07), (SALICYLIC ACIDTRIAMCINOLONE ACETONIDE:Hypertension:0.75), (SAMARIUM SM-153 LEXIDRONAM PENTASODIUM:Hypertension:0.5), (SARILUMAB:Hypertension:0.7), (SECOBARBITAL:Hyp ertension:0.48), (SECOBARBITAL SODIUM:Hypertension:0.72), (SELINEXOR:Hypertension:0.84), (SERMORELIN ACETATE:Hypertension:0.72), (SERTINDOLE:Hypertension:1.08), (SESAME OIL:Hypertension:0.98), (SHEA BUTTER:Hypertension:0.31), (SILTUXIMAB:Hypertension:1.02), (SIMEPREVIR SODIUM:Hypertension:1.15), (SOBUZOXANE:Hypertension:0.91), (SODIUM BENZOATESODIUM PHENYLACETATE:Hypertension:0.54), (SODIUM FLUORIDEVITAMINS:Hypertension:0.97), (SODIUM SALICYLATE:Hypertension:0.75), (SODIUM SULFIDE:Hypertension:0.49), (SOLIFENACINTAMSULOSIN:Hypertension:0.97), (SORAFE NIB TOSYLATE:Hypertension:0.73), (SPIRULINA:Hypertension:1.39), (STARCH, CORN:Hypertension:0.69), (STIRIPENTOL:Hypertension:0.43), (STREPTOMYCIN SULFATE:Hypertension:1.11), (STREPTOZOCIN:Hypertension:0.91), (STRONTIUM CHLORIDE:Hypertension:0.95), (STRONTIUM CHLORIDE SR-89:Hypertension:0.79), (SUCCIMER:Hypertension:0.33), (SULTHIAME:Hypertension:0.49), (S UPLATAST TOSILATE:Hypertension:1.78), (TANESPIMYCIN:Hypertension:0.78), (TAR:Hypertension:0.93), (TECHNETIUM TC-99M:Hypertension:0.26), (TECHNETIUM TC-99M EXAMETAZIME:Hypertension:0.47), (TECHNETIUM TC-99M MEBROFENIN:Hypertension:0.26), (TECHNETIUM TC-99M SODIUM PERTECHNETATE:Hypertension:0.3), (TECHNETIUM TC-99M SULFUR COLLOID:Hypertension:0), (TEDIZOLID:Hypertension:0.82), (TEMOCILLIN:Hypertension:0.58), (TENOFOVIR ALAFENAMIDE:Hypertension:0.37), (TENOFOVIR DISOPROXIL:Hypertension:0.65), (TERIZIDONE:Hypertension:0.87), (TESTOSTERONE DECANOATETESTOSTERONE ISOCAPROATETESTOSTERONE PHENYLPROPIONATETESTOSTERONE PROPIONATE:Hypertension:0.89), (TETANUS TOXOIDS:Hypertension:0.49), (TETRACAINE:Hypertension:0.77), (TETRAHYDROZOLINE: Hypertension:1.01), (THEANINE:Hypertension:0.63), (THIAMYLAL:Hypertension:0.65), (THIO GUANINE:Hypertension:0.51), (THIOGUANINE ANHYDROUS:Hypertension:0.34), (THIORIDAZINE HYDROCHLORIDE:Hypertension:0.76), (THROMBIN HUMAN:Hypertension:0.49), (THYMOL:Hypertension:1.33), (THYROGLOBULIN:Hypertensi on:0.94), (TICARCILLIN:Hypertension:0.43), (TISAGENLECLEUCEL:Hypertension:0.99), (TIS AGENLECLEUCEL-T:Hypertension:0.57), (TITANIUM DIOXIDE:Hypertension:0.21), (TOBRAMYCIN SULFATE:Hypertension:1.1), (TOLMETIN:Hypertension:0.41), (TOREMIFENE:Hypertension: 0.93), (TOSITUMOMABTOSITUMOMAB I-131:Hypertension:0.62), (TRANYLCYPROMINE:Hypertension:0.75), (TREBANANIB:Hyperte nsion:0.88), (TRENBOLONE:Hypertension:0.42), (TRIBENOSIDE:Hypertension:1.07), (TRIEN TINE:Hypertension:0.94), (TRIENTINE HYDROCHLORIDE:Hypertension:0.68), (TRISODIUM CITRATE DIHYDRATE:Hypertension:0.39), (TROLAMINE:Hypertension:1.16), (TROMETHAMINE:Hy pertension:1.23), (TROPATEPINE:Hypertension:0.31), (TRYPAN BLUE:Hypertension:0.39), (TRYPSIN:Hypertension:0.99), (TRYPTOPHAN:Hypertension:1.16), (TYPHOID VACCINE:Hypertension:0.75), (ULIPRISTAL:Hypertension:0.7), (UNDECYLENIC ACID:Hypertension:1.12), (URACIL:Hypertension:1.37), (URELUMAB:Hypertension:0.92), (U RIDINE TRIACETATE:Hypertension:0.52), (VADASTUXIMAB TALIRINE:Hypertension:0.87), (VALERIAN EXTRACT:Hypertension:0.79), (VELPATASVIR:Hypertension:0.77), (VERUPREVIR:Hyperten sion:0.85), (VINDESINE:Hypertension:0.72), (VINTAFOLIDE:Hypertension:0.97), (VON WILLEBRAND FACTOR (RECOMBINANT):Hypertension:0.31), (WHEAT GLUTEN:Hypertension:0.66), (WINE:Hypertension:0.18), (YELLOW FEVER VIRUS STRAIN 17D-204 LIVE ANTIGEN:Hypertension:0.1), (ZINC ACETATE:Hypertension:1.26), (ZINC ACETATE ANHYDROUSZINC GLUCONATE:Hypertension:0.54), (ZINC ACETATEZINC GLUCONATE:Hypertension:0.52), (ZINC CITRATE:Hypertension:1.37), (ZINC PICOLINATE:Hypertension:0.86), (ZOLEDRONATE DISODIUM:Hypertension:1.2), (ZUCLOPENTHIXOL HYDROCHLORIDE:Hypertension:0.89), (CELECOXIB:Hypertensive heart disease:0.27), (DEXLANSOPRAZOLE:Hypertensive heart disease:0.07), (DICLOFENAC:Hypertensive heart disease:0.16), (DICLOFENAC SODIUM:Hypertensive heart disease:0.35), (ENOXAPARIN:Hypertensive heart disease:0.08), (EVEROLIMUS:Hypertensive heart disease:0.33), (EVOLOCUMAB:Hypertensive heart disease:0.53), (FLECAINIDE:Hypertensive heart disease:0.35), (GLIPIZIDE:Hypertensive heart disease:0.26), (GLYBURIDE:Hypertensive heart disease:0.16), (INDAPAMIDEPERINDOPRIL:Hypertensive heart disease:0.25), (INSULIN HUMAN:Hypertensive heart disease:0.43), (ISOSORBIDE:Hypertensive heart disease:0.04), (NAPROXEN:Hypertensive heart disease:0.32), (OXYGEN:Hypertensive heart disease:0.84), (POTASSIUM:Hypertensive heart disease:0.37), (PRAVASTATIN:Hypertensive heart disease:0.27), (SACUBITRILVALSARTAN:Hypertensive heart disease:0.76), (UNSPECIFIED INGREDIENT:Hypertensive heart disease:0.44), (.ALPHA.1-PROTEINASE INHIBITOR HUMAN:Hyperthyroidism:0), (ACETAMINOPHENOXYCODONEOXYCODONE HYDROCHLORIDE:Hyperthyroidism:0.5), (ACETAMINOPHENPROPOXYPHENE HYDROCHLORIDE:Hyperthyroidism:0.28), (ACETAZOLAMIDE:Hyperthyroidism:1.12), (AD O-TRASTUZUMAB EMTANSINE:Hyperthyroidism:0.91), (AGALSIDASE BETA:Hyperthyroidism:0.39), (ALCOHOL:Hyperthyroidism:0.46), (ALFUZOSIN HYDROCHLORIDE:Hyperthyroidism:0.14), (ALISKIREN HEMIFUMARATE:Hyperthyroidism:0.25), (AMANTADINE:Hyperthyroidism:0.45), (AMANT ADINE HYDROCHLORIDE:Hyperthyroidism:0.13), (AMLODIPINEVALSARTAN:Hyperthyroidism: 0.35), (AZELASTINE:Hyperthyroidism:0.23), (AZITHROMYCIN DIHYDRATE:Hyperthyroidism:0.63), (BICALUTAMIDE:Hyperthyroidism:0.35), (BUDESONI DEFORMOTEROL FUMARATE:Hyperthyroidism:0.75), (BUTYLSCOPOLAMINE BROMIDE:Hyperthyroidism:0.9), (CALCIPOTRIENE:Hyperthyroidism:0.13), (CALCIUMCH OLECALCIFEROL:Hyperthyroidism:0.23), (CARVEDILOL PHOSPHATE:Hyperthyroidism:1.56), (CEFOTAXIME SODIUM:Hyperthyroidism:0.32), (CETIRIZINE HYDROCHLORIDEPSEUDOEPHEDRINE HYDROCHLORIDE:Hyperthyroidism:0.15), (CHLORDIAZEPOXIDE HYDROCHLORIDECLIDINIUM BROMIDE:Hyperthyroidism:0.19), (CIMETIDINE:Hyperthyroidism:0.97), (CLINDAMYCINC LINDAMYCIN PHOSPHATE:Hyperthyroidism:1.58), (CLOSTRIDIUM BUTYRICUM SPORES STRAIN M-55:Hyperthyroidism:0.2), (CLOTRIMAZOLE:Hyperthyroidism:1.34), (CORTICOTROPIN:Hype rthyroidism:0.39), (CYAMEMAZINE:Hyperthyroidism:0.02), (DACLATASVIR:Hyperthyroidis m:0.06), (DACLIZUMAB:Hyperthyroidism:0.44), (DALTEPARIN:Hyperthyroidism:0.53), (DAN AZOL:Hyperthyroidism:0.39), (DESONIDE:Hyperthyroidism:0.02), (DOXEPIN HYDROCHLORIDE:Hyperthyroidism:0.41), (ECONAZOLE:Hyperthyroidism:0.52), (ELETRIP TAN HYDROBROMIDE:Hyperthyroidism:1.4), (EMPAGLIFLOZINLINAGLIPTIN:Hyperthyroidis m:1.28), (EMPAGLIFLOZINMETFORMIN HYDROCHLORIDE:Hyperthyroidism:0.06), (ERYTHROMYCIN:Hyperthyroidism:0.72), (FEN OTEROL HYDROBROMIDEIPRATROPIUM BROMIDE:Hyperthyroidism:0.07), (FERROUS SULFATEGLYCINE:Hyperthyroidism:0.03), (FEXOFENADINEPSEUDOEPHEDRINE:Hype rthyroidism:0.08), (FISH OIL\OMEGA-3 FATTY ACIDS:Hyperthyroidism:0.24), (FLUDROCORTISONE ACETATE:Hyperthyroidism:0.69), (FLUOCINONIDE:Hyperthyroidism:0.27), (FORMOTEROL FUMARATEMOMETASONE FUROATE:Hyperthyroidism:0.08), (FUSIDIC ACID:Hyperthyroidism:0.63), (GIMERACILOTERACILTEGAFUR:Hyperthyroidism:0.1), (G LUCOSAMINE:Hyperthyroidism:0.4), (GLYCOPYRROLATE:Hyperthyroidism:0.05), (HYDR OXYCHLOROQUINE DIPHOSPHATE:Hyperthyroidism:0.19), (IDELALISIB:Hyperthyroidism:0.24), (IMIPRAMINE HYDROCHLORIDE:Hyperthyroidism:0.78), (INFLIXIMAB-DYYB:Hyperthyroidism:0.18), (INSULIN BEEF:Hyperthyroidism:0.25), (INTERFERON ALFA-2A:Hyperthyroidism:0.05), (INTERFERON ALFA-2B:Hyperthyroidism:0.06), (IRINOTECAN HYDROCHLORIDE:Hyperthyroidism:0.49), (ISOPROPYL ALCOHOLLEUPROLIDE ACETATENORETHINDRONE ACETATE:Hyperthyroidism:0.04), (IXAZOMIB:Hyperthyroidism:0.38), (KETOPROFEN:Hyper thyroidism:0.36), (KETOROLACKETOROLAC TROMETHAMINE:Hyperthyroidism:0.04), (LACTASE:Hyperthyroidism:0.17), (LOMITAPIDE MESYLATE:Hyperthyroidism:0.62), (LOTEPREDNOL ETABONATE:Hyperthyroidism:0.01), (METHENAMINE HIPPURATE:Hyperthyroidism:0.83), (METHYLPREDNISOLONE ACETATE:Hyperthyroidism:0.82), (MIDODRINE HYDROCHLORIDE:Hyperthyroidism:0.1), (MOXIFLOXACIN HYDROCHLORIDE:Hyperthyroidism:1.41), (MUPIROCINMUPIROCIN CALCIUM:Hyperthyroidism:0.37), (NICARDIPINE:Hyperthyroidism:0.4), (NIRAPARIB:Hyper thyroidism:0.47), (OCTREOTIDE:Hyperthyroidism:0.66), (OLAPARIB:Hyperthyroidism:0.1), (O SPEMIFENE:Hyperthyroidism:0.41), (OXYBUTYNIN CHLORIDE:Hyperthyroidism:0.8), (PAMIDRONATE DISODIUM:Hyperthyroidism:0.23), (PANTOPRAZOLE MAGNESIUM:Hyperthyroidism:1.59), (PAROXETINE:Hyperthyroidism:1.38), (PEMETREXE D DISODIUM:Hyperthyroidism:0.89), (PHENYTOIN:Hyperthyroidism:0.81), (PHENYTOIN SODIUM:Hyperthyroidism:0.35), (PHYTONADIONE:Hyperthyroidism:0.34), (PIMOBENDAN: Hyperthyroidism:0.14), (PONATINIB:Hyperthyroidism:1.14), (PRAMIPEXOLE DIHYDROCHLORIDE:Hyperthyroidism:1.12), (PRAZOSIN HYDROCHLORIDE:Hyperthyroidism:0.21), (PREDNISOLONEPREDNISOLONE ACETATEPREDNISOLONE SODIUM SUCCINATE:Hyperthyroidism:0.7), (REGORAFENIB:Hyperthyroidism:0.98), (RIFAXIMIN:H yperthyroidism:0.07), (RIOCIGUAT:Hyperthyroidism:0.28), (ROFLUMILAST:Hyperthyroidism:0.84), (ROSIGLITAZONE MALEATE:Hyperthyroidism:0.05), (SCOPOLAMINE:Hyperthyroidism:1.05), (SELEXIPAG:Hy perthyroidism:0.02), (SODIUM PHOSPHATE, DIBASICSODIUM PHOSPHATE, MONOBASIC:Hyperthyroidism:0.02), (SULFAMETHOXAZOLE:Hyperthyroidism:0.56), (TEL OTRISTAT ETHYL:Hyperthyroidism:0.15), (TERAZOSIN HYDROCHLORIDE:Hyperthyroidism:0.12), (TERBINAFINE HYDROCHLORIDE:Hyperthyroidism:0.44), (TERBUTALINE:Hyperthyroidism:0.22), (TERBU TALINE SULFATE:Hyperthyroidism:0.82), (TESTOSTERONE UNDECANOATE:Hyperthyroidism:0.4), (THIOCTIC ACID:Hyperthyroidism:0.22), (TIOTROPIUM:Hyperthyroidism:0.92), (TROSPIUM CHLORIDE:Hyperthyroidism:0.37), (UMECLIDINIUM BROMIDE:Hyperthyroidism:0.13), (VITAMIN A:Hyperthyroidism:0.45), (ZINC:Hyperthyroidism:0.62), (ATORVASTATIN CALCIUM:Hypertonia:0.09), (CALCIUM CARBONATECHOLECALCIFEROL:Hypertonia:0.02), (IMATINIB MESYLATE:Hypertonia:0.34), (OXCARBAZEPINE:Hypertonia:0.46), (SACUBITRILVALSA RTAN:Hypertonia:0.44), (WARFARIN:Hypertonia:0.32), (.ALPHA.1-PROTEINASE INHIBITOR HUMAN:Hypertonic bladder:0.18), (ABACAVIR SULFATEDOLUTEGRAVIR SODIUMLAMIVUDINE:Hypertonic bladder:0.31), (ACETAMINOPHENCAFFEINE:Hypertonic bladder:0.11), (ACETAZOLAMIDE:Hypertonic bladder:2.08), (ACITRETIN:Hypertonic bladder:0.09), (ACTIVATED CHARCOAL:Hypertonic bladder:0.48), (ADENINE:Hypertonic bladder:0.42), (AGALSIDASE BETA:Hypertonic bladder:1.23), (ALBIGLUTIDE:Hypertonic bladder:0.5), (ALCOHOL:Hypertonic bladder:0.51), (ALISKIREN HEMIFUMARATE:Hypertonic bladder:0.78), (AMBROXOLCLENBUTEROL:Hypertonic bladder:0.28), (AMISULPRIDE:Hypertonic bladder:0.98), (AMLODIPINE BESYLATEHYDROCHLOROTHIAZIDEVALSARTAN:Hypertonic bladder:0.61), (AMLODIPINEVALSARTAN:Hypertonic bladder:0.67), (AMMONIUM LACTATE:Hypertonic bladder:0.35), (AMOXAPINE:Hypertonic bladder:0.58), (AMPICILLIN SODIUMSULBACTAM SODIUM:Hypertonic bladder:0.47), (ANTIHISTAMINES NOS:Hypertonic bladder:0.24), (APOMORPHINE:Hypertonic bladder:0.03), (ARIPIPRAZOLE LAUROXIL:Hypertonic bladder:0.45), (ATOVAQUONE:Hypertonic bladder:0.25), (BECLOMETHASONEFORMOTEROL:Hypertonic bladder:0.12), (BETAMETHASONE VALERATE:Hypertonic bladder:0.37), (BLONANSERIN:Hypertonic bladder:0.11), (BUCILLAMINE:Hypertonic bladder:0.7), (BUDESONIDEFORMOTEROL FUMARATE:Hypertonic bladder:1.01), (BUPRENORPHINE HYDROCHLORIDE:Hypertonic bladder:0.95), (BUPRENORPHINE HYDROCHLORIDENALOXONE HYDROCHLORIDE:Hypertonic bladder:0.81), (BUTYLSCOPOLAMINE BROMIDE:Hypertonic bladder:0.51), (CALCITONIN:Hypertonic bladder:0.35), (CALCIUM CITRATEVITAMIN D:Hypertonic bladder:0.31), (CANAGLIFLOZINMETFORMIN HYDROCHLORIDE:Hypertonic bladder:1.23), (CANAKINUMAB:Hypertonic bladder:0.41), (CANNABIS SATIVA SUBSP. INDICA TOP:Hypertonic bladder:0.29), (CAPSAICIN:Hypertonic bladder:0.04), (CARBIDOPA:Hypertonic bladder:0.54), (CARFILZOMIB:Hypertonic bladder:0.92), (CETIRIZINE HYDROCHLORIDEPSEUDOEPHEDRINE HYDROCHLORIDE:Hypertonic bladder:0.64), (CETUXIMAB:Hypertonic bladder:0.44), (CHIMAPHILA UMBELLATAMANGANESE CHLORIDEPOPULUS TREMULOIDESSODIUM TAUROCHOLATE:Hypertonic bladder:0.01), (CHLORHEXIDINE:Hypertonic bladder:0.06), (CHLORPHENIRAMINEHYDROCODONE:Hypertonic bladder:0.21), (CIPROFIBRATE:Hypertonic bladder:0.3), (CLIKSTAR:Hypertonic bladder:0.21), (CODEINE PHOSPHATEGUAIFENESIN:Hypertonic bladder:0.44), (COLESTILAN CHLORIDE:Hypertonic bladder:0.11), (DABIGATRAN:Hypertonic bladder:0.88), (DALTEPARIN:Hypertonic bladder:0.56), (DALTEPARIN SODIUM:Hypertonic bladder:0.55), (DANAZOL:Hypertonic bladder:0.48), (DAPAGLIFLOZIN PROPANEDIOLMETFORMIN HYDROCHLORIDE:Hypertonic bladder:0.09), (DAPSONE:Hypertonic bladder:0.04), (DEFLAZACORT:Hypertonic bladder:1.47), (DESONIDE:Hypertonic bladder:0.74), (DEXTROMETHORPHAN HYDROBROMIDE:Hypertonic bladder:0.04), (DIGITOXIN:Hypertonic bladder:0.26), (DILAZEP:Hypertonic bladder:0.78), (DIPHENIDOL HYDROCHLORIDE:Hypertonic bladder:0.6), (DIPYRONE:Hypertonic bladder:0.17), (DOXYLAMINE SUCCINATE:Hypertonic bladder:0.2), (DROSPIRENONEETHINYL ESTRADIOL:Hypertonic bladder:0.79), (ECONAZOLE:Hypertonic bladder:0.17), (EDOXABAN TOSILATE MONOHYDRATE:Hypertonic bladder:0.29), (ELOTUZUMAB:Hypertonic bladder:0.05), (EMPAGLIFLOZINLINAGLIPTIN:Hypertonic bladder:0.95), (ETHINYL ESTRADIOLNORETHINDRONE:Hypertonic bladder:0.35), (ETRETINATE:Hypertonic bladder:1.09), (FENOTEROL HYDROBROMIDE:Hypertonic bladder:0.06), (FERROUS BISGLYCINATE:Hypertonic bladder:0.19), (FLURAZEPAMFLURAZEPAM HYDROCHLORIDE:Hypertonic bladder:0.26), (FOLATE SODIUM:Hypertonic bladder:0.03), (FORMOTEROL FUMARATEMOMETASONE FUROATE:Hypertonic bladder:0.03), (FOSINOPRIL:Hypertonic bladder:0.07), (FULVESTRANT:Hypertonic bladder:0.31), (FURSULTIAMINE:Hypertonic bladder:0.62), (FUSIDIC ACID:Hypertonic bladder:0.78), (GEMCITABINE HYDROCHLORIDE:Hypertonic bladder:0.01), (GLYBURIDEMETFORMIN HYDROCHLORIDE:Hypertonic bladder:0.03), (GLYCOPYRROLATE:Hypertonic bladder:0.4), (GLYCOPYRROLATEINDACATEROL MALEATE:Hypertonic bladder:0.23), (GUANABENZ ACETATE:Hypertonic bladder:0.12), (GUANFACINE:Hypertonic bladder:1), (GUANFACINE HYDROCHLORIDE:Hypertonic bladder:0.59), (HYDROCORTISONE ACETATE:Hypertonic bladder:0.21), (HYDROCORTISONE SODIUM SUCCINATE:Hypertonic bladder:0.18), (ILOPROST:Hypertonic bladder:0.59), (IMATINIB MESYLATE:Hypertonic bladder:1.5), (INDAPAMIDEPERINDOPRIL:Hypertonic bladder:1.3), (INFLIXIMAB-DYYB:Hypertonic bladder:0.13), (INSULIN BEEF:Hypertonic bladder:0.51), (INTERFERON:Hypertonic bladder:0.59), (INTERFERON ALFA-2A:Hypertonic bladder:0.36), (IRINOTECAN:Hypertonic bladder:0.29), (IRON SUCROSE:Hypertonic bladder:0.56), (IVABRADINE:Hypertonic bladder:0.06), (IXAZOMIB:Hypertonic bladder:0.13), (KETOROLACKETOROLAC TROMETHAMINE:Hypertonic bladder:0.43), (LAPATINIB DITOSYLATE:Hypertonic bladder:0.13), (LEDIPASVIRSOFOSBUVIR:Hypertonic bladder:2.16), (LEVOCARNITINE HYDROCHLORIDE:Hypertonic bladder:0.11), (LEVOLEUCOVORIN CALCIUM:Hypertonic bladder:0.29), (LIDOCAINEPRILOCAINE:Hypertonic bladder:0.71), (LISDEXAMFETAMINE DIMESYLATE:Hypertonic bladder:1.67), (LUTETIUM LU-177:Hypertonic bladder:0.01), (MAXACALCITOL:Hypertonic bladder:0.43), (MEPOLIZUMAB:Hypertonic bladder:0.07), (MESNA:Hypertonic bladder:0.04), (METAMIZOLE:Hypertonic bladder:0.74), (METFORMIN HYDROCHLORIDESAXAGLIPTIN HYDROCHLORIDE:Hypertonic bladder:0.13), (METFORMIN HYDROCHLORIDEVILDAGLIPTIN:Hypertonic bladder:0.86), (METHYLPREDNISOLONE ACETATE:Hypertonic bladder:1.69), (METRELEPTIN:Hypertonic bladder:0.45), (MIZORIBINE:Hypertonic bladder:0.83), (MOEXIPRIL HYDROCHLORIDE:Hypertonic bladder:0.34), (MORPHINE SULFATENALTREXONE HYDROCHLORIDE:Hypertonic bladder:0.01), (MOXONIDINE:Hypertonic bladder:0.39), (NADROPARIN CALCIUM:Hypertonic bladder:0.2), (NALOXONE HYDROCHLORIDETILIDINE HYDROCHLORIDE:Hypertonic bladder:0.48), (NIRAPARIB:Hypertonic bladder:0.52), (OLMESARTAN:Hypertonic bladder:0.05), (OPIPRAMOL:Hypertonic bladder:0.01), (PANCRELIPASE:Hypertonic bladder:0.19), (PANITUMUMAB:Hypertonic bladder:0), (PATIROMER:Hypertonic bladder:0.48), (PAZOPANIB HYDROCHLORIDE:Hypertonic bladder:1.77), (PEMETREXED:Hypertonic bladder:0.2), (PEMETREXED DISODIUM:Hypertonic bladder:1.07), (PETROLATUM:Hypertonic bladder:0.13), (PHYTONADIONE:Hypertonic bladder:0.21), (PIMOBENDAN:Hypertonic bladder:0.44), (PIOGLITAZONE:Hypertonic bladder:0.58), (POLYETHYLENE GLYCOL 3350\POTASSIUM CHLORIDESODIUM BICARBONATESODIUM CHLORIDESODIUM SULFATE ANHYDROUS:Hypertonic bladder:0.31), (POLYETHYLENE GLYCOL 4000:Hypertonic bladder:0.02), (PONATINIB:Hypertonic bladder:0.91), (POTASSIUM CITRATETRISODIUM CITRATE DIHYDRATE:Hypertonic bladder:0.42), (PROPAFENONE:Hypertonic bladder:0.66), (PROTRIPTYLINE HYDROCHLORIDE:Hypertonic bladder:0.56), (RADIATION THERAPY:Hypertonic bladder:0.17), (RILPIVIRINE HYDROCHLORIDE:Hypertonic bladder:0.45), (RIZATRIPTAN:Hypertonic bladder:0.6), (SARPOGRELATE HYDROCHLORIDE:Hypertonic bladder:0.27), (SEVELAMER CARBONATE:Hypertonic bladder:0.2), (SODIUM PHOSPHATE, DIBASICSODIUM PHOSPHATE, MONOBASIC:Hypertonic bladder:0.28), (SOFOSBUVIRVELPATASVIR:Hypertonic bladder:0.51), (SULFAMETHOXAZOLE:Hypertonic bladder:0.84), (TETRACYCLINE:Hypertonic bladder:1.24), (TIOTROPIUM BROMIDE:Hypertonic bladder:0.08), (TRIHEXYPHENIDYL:Hypertonic bladder:0.02), (TRIHEXYPHENIDYL HYDROCHLORIDE:Hypertonic bladder:0.03), (ZILEUTON:Hypertonic bladder:0.25), (ACETAMINOPHENCODEINE PHOSPHATE:Hypertriglyceridaemia:0.87), (AMLODIPINE BESYLATEOLMESARTAN MEDOXOMIL:Hypertriglyceridaemia:0.16), (ANASTROZOLE:Hypertriglyceridaemia:0.87), (A TROPINE SULFATEDIPHENOXYLATE HYDROCHLORIDE:Hypertriglyceridaemia:0.2), (BISACODYL OR DOCUSATE SODIUM:Hypertriglyceridaemia:0.41), (BUDESONIDE:Hypertriglyceridaemia:1.16), (BUMET ANIDE:Hypertriglyceridaemia:1.15), (BUPRENORPHINE HYDROCHLORIDE:Hypertriglyceridaemia:0.01), (BUPRENORPHINE HYDROCHLORIDENALOXONE HYDROCHLORIDE:Hypertriglyceridaemia:0.07), (CLINDAMYCINCLINDAMYCIN PHOSPHATE:Hypertriglyceridaemia:0.14), (CORTISONEHYDROCORTISONE:Hypertriglyce ridaemia:0.27), (CYCLOBENZAPRINE:Hypertriglyceridaemia:0.19), (DASABUVIROMBITAS VIRPARITAPREVIRRITONAVIR:Hypertriglyceridaemia:0.01), (DASATINIB:Hypertriglycer idaemia:0.8), (DICYCLOMINE HYDROCHLORIDE:Hypertriglyceridaemia:0.31), (DIGITOXIN:Hypertriglyceridaemia:0.01), (DOFETILIDE:Hypertriglyceridaemia:0.49), (DULAGLUTIDE:Hypertriglyceridaemia:0.43), (E MPAGLIFLOZINLINAGLIPTIN:Hypertriglyceridaemia:0.68), (EMPAGLIFLOZINMETFOR MIN HYDROCHLORIDE:Hypertriglyceridaemia:0.33), (ETORICOXIB:Hypertriglyceridaemia:0.1), (FESOTERODINE FUMARATE:Hypertriglyceridaemia:0.22), (FLUTICASONE FUROATEVILANTEROL TRIFENATATE:Hypertriglyceridaemia:0.05), (GLYCOPYRRONIUMINDACATEROL:Hypert riglyceridaemia:0.1), (HEPARIN SODIUM:Hypertriglyceridaemia:0.71), (HYDROXYCHLOROQUINE SULFATE:Hypertriglyceridaemia:0.56), (ILOPROST:Hypertriglyceridaemia:0.22), (LABETAL OLLABETALOL HYDROCHLORIDE:Hypertriglyceridaemia:0.26), (LERCANIDIPINE HYDROCHLORIDE:Hypertriglyceridaemia:1.22), (LISDEXAMFETAMINE DIMESYLATE:Hypertriglyceridaemia:0.32), (MELATONIN:Hypertriglyceridaemia:0.14), (ME TAXALONE:Hypertriglyceridaemia:0.21), (METFORMIN HYDROCHLORIDEROSIGLITAZONE MALEATE:Hypertriglyceridaemia:0.2), (MIDODRINE:Hypertriglyceridaemia:0.08), (MOXIFL OXACIN HYDROCHLORIDE:Hypertriglyceridaemia:0), (NADOLOL:Hypertriglyceridaemia:0.46), (NIT ROFURANTOIN:Hypertriglyceridaemia:0.29), (PANTOPRAZOLE MAGNESIUM:Hypertriglyceridaemia:0.42), (PAROXETINE:Hypertriglyceridaemia:0.46), (PAZ OPANIB HYDROCHLORIDE:Hypertriglyceridaemia:0.78), (PHENPROCOUMON:Hypertriglyceridaemi a:0.12), (PONATINIB:Hypertriglyceridaemia:0.19), (PROBIOTICS NOS:Hypertriglyceridaemia:0.37), (ROFLUMILAST:Hypertriglyceridaemia:0.07), (ROSIGLITA ZONE MALEATE:Hypertriglyceridaemia:0.31), (SACUBITRILVALSARTAN:Hypertriglyceridaemia: 0.72), (TERIFLUNOMIDE:Hypertriglyceridaemia:0.01), (TETRACYCLINE:Hypertriglyceridae mia:0.22), (TIOTROPIUM:Hypertriglyceridaemia:0.11), (TOCILIZUMAB:Hypertriglyceridaemi a:0.25), (UMECLIDINIUM BROMIDEVILANTEROL TRIFENATATE:Hypertriglyceridaemia:0.03), (VALGANCICLOVIR HYDROCHLORIDE:Hypertriglyceridaemia:0.06), (VITAMIN B:Hypertriglyceridaemia:0.38), (ENALAPRIL:Hypertrophic cardiomyopathy:0.49), (EVOLOCUMAB:Hypertrophic cardiomyopathy:0.14), (LOVASTATIN:Hypertrophic cardiomyopathy:0.04), (NAPROXEN:Hypertrophic cardiomyopathy:0.25), (SACUBITRILVALSARTAN:Hypertrophic cardiomyopathy:0.05), (TORSEMIDE:Hypertrophic cardiomyopathy:0.44), (.ALPHA.1-PROTEINASE INHIBITOR HUMAN:Hyperuricaemia:0.44), (ACETAMINOPHENAPRONALIDECAFFEINEPROPYPH ENAZONE:Hyperuricaemia:1.46), (ACETAMINOPHENBROMISOVALDIHYDROCODEIN E PHOSPHATEDIPHENHYDRAMINE SALICYLATEDYPHYLLINEMETHYLEPHEDRINE HYDROCHLORIDE, (+/−)-:Hyperuricaemia:0.78), (ACETAMINOPHENCAFFEINE:Hyperuricaemia:0.44), (ACETA MINOPHENPSEUDOEPHEDRINE HYDROCHLORIDE:Hyperuricaemia:0.6), (ACOTIAMIDE HYDROCHLORIDE:Hyperuricaemia:0.34), (ADENOSINE TRIPHOSPHATE DISODIUM:Hyperuricaemia:0.15), (ADO-TRASTUZUMAB EMTANSINE:Hyperuricaemia:1.37), (ADUCANUMAB:Hyperuricaemia:0.16), (ALBUTEROLI PRATROPIUM:Hyperuricaemia:0.27), (ALECTINIB:Hyperuricaemia:0.35), (ALISKIREN HEMIFUMARATE:Hyperuricaemia:0.17), (ALUMINUM SILICATE:Hyperuricaemia:1.63), (AMANTADINE:Hyperuricaemia:0.74), (AMINO ACIDSCARBOHYDRATESMINERALSSOYBEAN OILVITAMINS:Hyperuricaemia:1.18), (AMMONIUM LACTATE:Hyperuricaemia:0.01), (AMPHETAMINE ASPARTATEAMPHETAMINE SULFATEDEXTROAMPHETAMINE SACCHARATEDEXTROAMPHETAMINE SULFATE:Hyperuricaemia:0.32), (AMPICILLIN SODIUM:Hyperuricaemia:0.91),(ARMODAFINIL:Hyperuricaemia:0.17), (AROTINOLOL:Hyp eruricaemia:0.31), (ASCORBIC ACIDBIOTINCALCIUM PANTOTHENATECOBALAMINFOLIC ACIDNIACINAMIDEPYRIDOXINE HYDROCHLORIDERIBOFLAVINTHIAMINE MONONITRATE:Hyperuricaemia:0.11), (ASPIRINDIHYDROXYALUMINUM AMINOACETATEMAGNESIUM CARBONATE:Hyperuricaemia:0.63), (ASPIRINMAGNESIUM OXIDE:Hyperuricaemia:1.08), (ATAZANAVIR:Hyperuricaemia:0.49), (AZELASTINE:Hyperur icaemia:0.07), (AZITHROMYCIN OR METHOTREXATE SODIUM:Hyperuricaemia:0.14), (BECLOMETHASONE:Hyperuricaemia:0.42), (BELATACEP T:Hyperuricaemia:0.46), (BENAZEPRILBENAZEPRIL HYDROCHLORIDE:Hyperuricaemia:0.94), (BETAMETHASONE DIPROPIONATECALCIPOTRIENECALCIPOTRIENE HYDRATE:Hyperuricaemia:0.27), (BETAMETHASONE SODIUM PHOSPHATENEOMYCIN SULFATE:Hyperuricaemia:1.46), (BIPERIDEN:Hyperuricaemia:0.15), (BRIMONIDINE TARTRATETIMOLOL MALEATE:Hyperuricaemia:0.13), (BROMPERIDOL:Hyperuricaemia:0.27), (BUDESONIDEF ORMOTEROL:Hyperuricaemia:0.1), (BUFORMIN:Hyperuricaemia:0.84), (BUPRENORPHINE HYDROCHLORIDENALOXONE HYDROCHLORIDE:Hyperuricaemia:0.23), (BUPROPION HYDROCHLORIDE:Hyperuricaemia:1.46), (BUSPIRONE HYDROCHLORIDE:Hyperuricaemia:0.83), (CABAZITAXEL:Hyperuricaemia:0.75), (CABOZ ANTINIB:Hyperuricaemia:0.16), (CALCITONIN SALMON:Hyperuricaemia:0.56), (CALCIUM CARBONATEERGOCALCIFEROLRETINOL:Hyperuricaemia:0.26), (CALCIUM CARBONATEVITAMIN D:Hyperuricaemia:0.09), (CALCIUM CITRATE:Hyperuricaemia:0.62), (CALCIUM GLUCONATEDEXTROSEMAGNESIUM SULFATEPOTASSIUM ACETATEPOTASSIUM PHOSPHATE, DIBASICZINC SULFATE:Hyperuricaemia:0.05), (CALCIUMMAGNESIUM:Hyperuricaemia:0.18), (CANAGL IFLOZINMETFORMIN HYDROCHLORIDE:Hyperuricaemia:1.08), (CARBIDOPA:Hyperuricaemia:0.27), (CARISOPR ODOL:Hyperuricaemia:0.13), (CARMUSTINE:Hyperuricaemia:0.72), (CARPRONIUM CHLORIDE:Hyperuricaemia:0.67), (CARVEDILOL PHOSPHATE:Hyperuricaemia:1.66), (CEFAZOLIN:Hyperuricaemia:1.56), (CEFOTAXIMECE FOTAXIME SODIUM:Hyperuricaemia:0.53), (CEFOTIAM HYDROCHLORIDE:Hyperuricaemia:1.04), (CEFTERAM PIVOXIL:Hyperuricaemia:0.98), (CHLORPROMAZINEPROMETHAZINE:Hyperuricaemia:0.62), (CHOLINE ALFOSCERATE:Hyperuricaemia:0.29), (CHONDROITIN SULFATE (CHICKEN):Hyperuricaemia:0.08), (CLADRIBINE:Hyperuricaemia:0.11), (CLINDAMYCIN HYDROCHLORIDE:Hyperuricaemia:0.07), (CLOFARABINE:Hyperuricaemia:0.44), (CLOXA CILLIN SODIUM:Hyperuricaemia:0.07), (CORTICOTROPIN:Hyperuricaemia:0.12), (CUPRIC SULFATEFERRIC CHLORIDEMANGANESE CHLORIDEPOTASSIUM IODIDEZINC SULFATE:Hyperuricaemia:0.62), (CYANAMIDE:Hyperuricaemia:0.59), (DABIGATRAN ETEXILATE:Hyperuricaemia:0.33), (DACLIZUMAB:Hyperuricaemia:0.88), (DAPAGLIFLOZI N PROPANEDIOL:Hyperuricaemia:0.78), (DAPAGLIFLOZIN PROPANEDIOLMETFORMIN HYDROCHLORIDE:Hyperuricaemia:0), (DASABUVIROMBITASVIRPARITAPREVIRRIT ONAVIR:Hyperuricaemia:0.18), (DEFERIPRONE:Hyperuricaemia:0.19), (DEXLANSOPRAZO LE:Hyperuricaemia:1.09), (DEXTRAN:Hyperuricaemia:0.12), (DICYCLOMINE HYDROCHLORIDE:Hyperuricaemia:0.76), (DIETARY SUPPLEMENT:Hyperuricaemia:0.03), (DIPYRONE:Hyperuricaemia:0.47), (DIVALPROEX SODIUM:Hyperuricaemia:1.13), (DOCUSATE CALCIUM:Hyperuricaemia:1.03), (DOFETILIDE:Hyperuricaemia:1.15), (DOLASETRON MESYLATE:Hyperuricaemia:0.14), (DORZOLAMIDE:Hyperuricaemia:0.7), (DUTASTERIDE TAMSULOSIN HYDROCHLORIDE:Hyperuricaemia:0.89), (ECONAZOLE:Hyperuricaemia:1.8), (ELETRIPTA N HYDROBROMIDE:Hyperuricaemia:0.6), (EMPAGLIFLOZINMETFORMIN HYDROCHLORIDE:Hyperuricaemia:0.14), (ENTACAPONE:Hyperuricaemia:0.21), (EPHEDRI NE HYDROCHLORIDE:Hyperuricaemia:0.17), (EPINEPHRINE:Hyperuricaemia:1.2), (ERIBULIN: Hyperuricaemia:0.32), (ERIBULIN MESYLATE:Hyperuricaemia:0.4), (ERYTHROMYCINSULFISOXAZOLE:Hyperuricaemia:0.2), (ESMOLOL HYDROCHLORIDE:Hyperuricaemia:0.22), (FENOTEROL HYDROBROMIDE:Hyperuricaemia:0.98), (FLOMOXEF:Hyperuricaemia:0.45), (FLOPROPIO NE:Hyperuricaemia:0.49), (FLUOCINONIDE:Hyperuricaemia:0.27), (FLUVOXAMINE MALEATE:Hyperuricaemia:0.39), (FORMOTEROL FUMARATE DIHYDRATEMOMETASONE FUROATE:Hyperuricaemia:0.59), (FORMOTEROL FUMARATEMOMETASONE FUROATE:Hyperuricaemia:0.42), (FOSFOMYCIN:Hyperuricaemia:0.81), (FOSINOPRIL:Hyper uricaemia:0.15), (FOSINOPRIL SODIUMHYDROCHLOROTHIAZIDE:Hyperuricaemia:0.03), (FUDOSTEINE:Hyperuricaemi a:1.08), (GARENOXACIN:Hyperuricaemia:0.52), (GLUCAGON:Hyperuricaemia:0.13), (GLUT AMINESODIUM GUALENATE:Hyperuricaemia:1.32), (GLYCERINPARAFFIN:Hyperuricaemia:0.49), (GLYC OPYRROLATEINDACATEROL MALEATE:Hyperuricaemia:0.35), (GUAIAZULENE:Hyperuricaemia:0.63), (HYDROCORTIS ONE PROBUTATE:Hyperuricaemia:0.58), (HYDROCORTISONE SODIUM PHOSPHATE:Hyperuricaemia:0.17), (HYDROXOCOBALAMINPYRIDOXINE HYDROCHLORIDETHIAMINE HYDROCHLORIDE:Hyperuricaemia:0.06), (IBRITUMOMAB TIUXETAN:Hyperuricaemia:1.99), (IMIGLUCERASE:Hyperuricaemia:0.7), (INFLIXIMAB-DYYB:Hyperuricaemia:0.13), (INSULIN PORKINSULIN PURIFIED PORK:Hyperuricaemia:0.07), (INTERFERON ALFA-2A:Hyperuricaemia:0.98), (INTERFERON ALFA-2B:Hyperuricaemia:0.1), (INTERFERON ALFA-N1:Hyperuricaemia:0.29), (ISONIAZIDPYRAZINAMIDERIFAMPIN:Hyperuricaemia:0.3), (I TOPRIDE HYDROCHLORIDE:Hyperuricaemia:0.01), (LABETALOL HYDROCHLORIDE:Hyperuricaemia:1.94), (LACIDIPINE:Hyperuricaemia:0.17), (LACOSAMI DE:Hyperuricaemia:0.4), (LANDIOLOL HYDROCHLORIDE:Hyperuricaemia:1.32), (LECITHINSODIUM BICARBONATESODIUM PHOSPHATE, MONOBASIC:Hyperuricaemia:0.21), (LENVATINIB:Hyperuricaemia:0.48), (LEVOSIMENDA N:Hyperuricaemia:0.52), (LICORICE ROOT EXTRACTRHUBARB:Hyperuricaemia:0.89), (LIDOCAINETRIBENOSIDE:Hyperuricaemia: 0.31), (LITHIUM:Hyperuricaemia:0.12), (LOBUCAVIR:Hyperuricaemia:0.7), (LOTEPREDNOL ETABONATE:Hyperuricaemia:0.01), (MAGNESIUM ASPARTATE:Hyperuricaemia:0.07), (MAGNESIUM ASPARTATE HYDROCHLORIDE:Hyperuricaemia:1.22), (MECLIZINE HYDROCHLORIDE:Hyperuricaemia:0.35), (MEPENZOLATE BROMIDE:Hyperuricaemia:1.07), (MEPERIDINE:Hyperuricaemia:1.44), (MEPOLIZUMAB:Hy peruricaemia:0.54), (METAXALONE:Hyperuricaemia:0.25), (METFORMIN HYDROCHLORIDESAXAGLIPTIN HYDROCHLORIDE:Hyperuricaemia:0.05), (METHADONE HYDROCHLORIDE:Hyperuricaemia:1.38), (METHENAMINE HIPPURATE:Hyperuricaemia:0.7), (METHENOLONE ACETATE:Hyperuricaemia:0.69), (MIGLUSTAT:Hyperuricaemia:0.36), (MIRIPLATIN:Hyperu ricaemia:0.63), (MOGAMULIZUMAB:Hyperuricaemia:0.06), (MORPHINE SULFATENALTREXONE HYDROCHLORIDE:Hyperuricaemia:0.29), (NIPRADILOL:Hyperuricaemia:0.17), (NITROFUR ANTOINNITROFURANTOIN MONOHYDRATE:Hyperuricaemia:0.13), (NOREPINEPHRINE BITARTRATE:Hyperuricaemia:0.49), (NORTRIPTYLINE:Hyperuricaemia:0.61), (OK-432:Hyperuricaemia:0.45), (OLAPARIB:Hyperuricaemia:0.06), (ORGOTEIN:Hyperuricaemia:0.22), (OZAGREL:Hyperuricaemia:0.6), (PALBOCICLIB:Hyperuricaemia:0.11), (PANTOPRAZOLE MAGNESIUM:Hyperuricaemia:1.74), (PATIROMER:Hyperuricaemia:0.32), (PAZUFLOXACIN MESILATE:Hyperuricaemia:0.71), (PEGINTERFERON BETA-1A:Hyperuricaemia:0.47), (PEGVISOMANT:Hyperuricaemia:0.22), (PENICILLIN:Hyperuricae mia:1.2), (PENICILLIN G:Hyperuricaemia:0.45), (PENICILLIN V POTASSIUM:Hyperuricaemia:0), (PENTOSTATIN:Hyperuricaemia:0.8), (PENTOXYVERINE CITRATE:Hyperuricaemia:0.21), (PHENOBARBITAL SODIUM:Hyperuricaemia:0.11), (PILOCARPINE:Hyperuricaemia:0.19), (PILSICAINIDE:Hype ruricaemia:0.22), (PIRENZEPINE:Hyperuricaemia:0.2), (PNEUMOCOCCAL VACCINE, POLYVALENT 23:Hyperuricaemia:0.26), (POLYETHYLENE GLYCOL 3350\POTASSIUM CHLORIDESODIUM BICARBONATESODIUM CHLORIDESODIUM SULFATE ANHYDROUS:Hyperuricaemia:0.2), (PORFIMER SODIUM:Hyperuricaemia:0.63), (POTASSIUM CHLORIDESODIUM CHLORIDESODIUM LACTATE:Hyperuricaemia:1.56), (PRANOPROFEN:Hyperuricaemia:0.42), (PRAZEPAM:Hype ruricaemia:0.05), (PRAZOSIN:Hyperuricaemia:0.24), (PRAZOSIN HYDROCHLORIDE:Hyperuricaemia:0.09), (PREDNISOLONE SODIUM PHOSPHATE:Hyperuricaemia:0.59), (PROPAFENONE HYDROCHLORIDE:Hyperuricaemia:0.7), (QUINACRINE:Hyperuricaemia:0.21), (RADIATIO N THERAPY:Hyperuricaemia:0.6), (RHUBARB:Hyperuricaemia:0.37), (ROSIGLITAZONE MALEATE:Hyperuricaemia:0.06), (ROXITHROMYCIN:Hyperuricaemia:0.72), (SCOPOLAMI NE HYDROBROMIDE:Hyperuricaemia:0.21), (SENNOSIDE A:Hyperuricaemia:0.17), (SENNOSIDE B:Hyperuricaemia:0.55), (SERRAPEPTASE:Hyperuricaemia:0.56), (SIVELESTAT SODIUM:Hyperuricaemia:1.28), (SOFOSBUVIRVELPATASVIR:Hyperuricaemia:0.06), (SOLI FENACIN:Hyperuricaemia:0.27), (SPIRAMYCIN:Hyperuricaemia:0.09), (SUMATRIPTAN:Hyp eruricaemia:0.21), (SUMATRIPTAN SUCCINATE:Hyperuricaemia:0.11), (SUPLATAST TOSILATE:Hyperuricaemia:1.92), (TALTIRELIN:Hyperuricaemia:0.21), (TECELEUKIN:Hyper uricaemia:0.45), (TEGAFURURACIL:Hyperuricaemia:0.64), (TERAZOSIN HYDROCHLORIDE:Hyperuricaemia:0.56), (TERIPARATIDE ACETATE:Hyperuricaemia:0.03), (TETRACYCLINE:Hyperuricaemia:0.82), (THROMBOMOD ULIN ALFA:Hyperuricaemia:1.51), (TIANEPTINE:Hyperuricaemia:0.04), (TIMOLOL MALEATE:Hyperuricaemia:0.52), (TIMOLOLTRAVOPROST:Hyperuricaemia:0.03), (TOFISO PAM:Hyperuricaemia:1.03), (TRABECTEDIN:Hyperuricaemia:0.1), (TRANILAST:Hyperuricae mia:0.37), (TRAPIDIL:Hyperuricaemia:0.1), (TRAVOPROST:Hyperuricaemia:0.34), (TRIHEXY PHENIDYL:Hyperuricaemia:0.38), (TULOBUTEROL HYDROCHLORIDE:Hyperuricaemia:0.63), (VASOPRESSIN:Hyperuricaemia:0.07), (VINORE LBINE TARTRATE:Hyperuricaemia:0.36), (VONOPRAZAN:Hyperuricaemia:1.38), (ZILEUTON:Hype ruricaemia:0.16), (ZINC:Hyperuricaemia:0.54), (ZOTEPINE:Hyperuricaemia:0.53), (ACETAMIN OPHENOXYCODONE HYDROCHLORIDEOXYCODONE TEREPHTHALATE:Hypoaesthesia:0.03), (ACETAZOLAMIDE:Hypoaesthesia:0.26), (ADO-TRASTUZUMAB EMTANSINE:Hypoaesthesia:0.6), (ALBUTEROL SULFATEIPRATROPIUM BROMIDE:Hypoaesthesia:1.01), (ALEMTUZUMAB:Hypoaesthesia:0.1), (AMANTADINE HYDROCHLORIDE:Hypoaesthesia:0.03), (BENAZEPRILBENAZEPRIL HYDROCHLORIDE:Hypoaesthesia:0.39), (BISACODYL OR DOCUSATE SODIUM:Hypoaesthesia:1.1), (BUPRENORPHINE HYDROCHLORIDE:Hypoaesthesia:0.13), (BUPRENORPHINE HYDROCHLORIDENALOXONE HYDROCHLORIDE:Hypoaesthesia:0.29), (CALCITONIN SALMON:Hypoaesthesia:0.21), (CALCIUM ACETATE:Hypoaesthesia:0.55), (CANDESARTAN CILEXETILHYDROCHLOROTHIAZIDE:Hypoaesthesia:0.71), (CAPTOPRIL:Hypoaesthesia: 0.3), (CARVEDILOL PHOSPHATE:Hypoaesthesia:0.98), (CEFTRIAXONE SODIUM:Hypoaesthesia:0.03), (CETUXIMAB:Hypoaesthesia:0.38), (CHLORTHALIDONE:Hy poaesthesia:0.58), (CORTICOTROPIN:Hypoaesthesia:0.01), (DACLIZUMAB:Hypoaesthesia:0.5 3), (DASATINIB:Hypoaesthesia:0.72), (DEFLAZACORT:Hypoaesthesia:0.2), (DESLORATADI NE:Hypoaesthesia:0.48), (DOXEPIN:Hypoaesthesia:0.12), (DRONABINOL:Hypoaesthesia:0.68), (EDOXABAN TOSYLATE:Hypoaesthesia:0.88), (EMPAGLIFLOZINLINAGLIPTIN:Hypoaesthesia:0.4), (ESTROGENS, CONJUGATED\MEDROXYPROGESTERONE ACETATE:Hypoaesthesia:0.55), (ETORICOXIB:Hypoaesthesia:0.01), (ETRETINATE:Hypoaest hesia:0.18), (FERROUS CITRATE:Hypoaesthesia:0.01), (FISH OIL\OMEGA-3 FATTY ACIDS:Hypoaesthesia:0.09), (FLUTICASONE FUROATE:Hypoaesthesia:0.12), (FULVESTRANT:Hypoaesthesia:0.95), (FUSIDIC ACID:Hypoaesthesia:0.05), (GADOLINIUM:Hypoaesthesia:0.53), (HALOPERIDOL:Hypoaesth esia:0.12), (HERBALSTURMERIC:Hypoaesthesia:0.59), (HYDROCHLOROTHIAZIDEVALS ARTAN:Hypoaesthesia:0.37), (HYDROXYCHLOROQUINE DIPHOSPHATE:Hypoaesthesia:0.07), (HYOSCYAMINE:Hypoaesthesia:0.41), (IBRUTINIB:Hy poaesthesia:1.23), (ICOSAPENT ETHYL:Hypoaesthesia:0.05), (ILOPROST:Hypoaesthesia:0.26), (INDAPAMIDE:Hypoaesthesia: 0.6), (INDOMETHACIN:Hypoaesthesia:0.34), (INSULIN DEGLUDEC:Hypoaesthesia:0.16), (IPRATROPIUM:Hypoaesthesia:0.66), (IRINOTECAN:Hypo aesthesia:0.03), (ISOSORBIDE:Hypoaesthesia:0.77), (LERCANIDIPINE:Hypoaesthesia:0.52), (L EUCOVORIN CALCIUM:Hypoaesthesia:0.24), (LEVALBUTEROL HYDROCHLORIDE:Hypoaesthesia:0.68), (LOMITAPIDE MESYLATE:Hypoaesthesia:0.18), (MECLIZINEMECLIZINE HYDROCHLORIDE:Hypoaesthesia:0.11), (METHENAMINE HIPPURATE:Hypoaesthesia:0.57), (METHYLPREDNISOLONE ACETATE:Hypoaesthesia:0.01), (MYCOPHENOLATE MOFETILMYCOPHENOLATE MOFETIL HYDROCHLORIDE:Hypoaesthesia:0.36), (NATEGLINIDE:Hypoaesthesia:0.08), (NILOTINIB: Hypoaesthesia:1.3), (OLANZAPINE:Hypoaesthesia:0.82), (PANTOPRAZOLE MAGNESIUM:Hypoaesthesia:0.57), (PAZOPANIB HYDROCHLORIDE:Hypoaesthesia:0.72), (PEGINTERFERON BETA-1A:Hypoaesthesia:0.5), (PENICILLIN:Hypoaesthesia:0.14), (PERINDOPRIL:Hypoaesthesia:0.36), (PHENYTOIN:Hypoaesthesia:0.08), (PILSICAINIDE HYDROCHLORIDE:Hypoaesthesia:0.04), (POLYETHYLENE GLYCOLS:Hypoaesthesia:1.34), (PONATINIB:Hypoaesthesia:0.18), (PROGESTERONE:Hypoa esthesia:0.25), (PROPAFENONE HYDROCHLORIDE:Hypoaesthesia:0.03), (RABEPRAZOLE:Hypoaesthesia:0.83), (RANOLAZ INE:Hypoaesthesia:1.21), (RIZATRIPTAN BENZOATE:Hypoaesthesia:0.52), (SCOPOLAMINE:Hypoaesthesia:0.84), (SULFAMETHOXA ZOLE:Hypoaesthesia:0.17), (SUMATRIPTANSUMATRIPTAN SUCCINATE:Hypoaesthesia:0.43), (TETRACYCLINE:Hypoaesthesia:0.1), (THALIDOMIDE:H ypoaesthesia:0.53), (TIOTROPIUM:Hypoaesthesia:0.09), (UMECLIDINIUM BROMIDEVILANTEROL TRIFENATATE:Hypoaesthesia:0.31), (VITAMIN A:Hypoaesthesia:0.03), (ANASTROZOLE:Hypoalbuminaemia:0.49), (ATORVASTATIN:Hypoa lbuminaemia:0.24), (ATOVAQUONE:Hypoalbuminaemia:0.07), (BENDAMUSTINE:Hypoalbu minaemia:0.17), (BERAPROST SODIUM:Hypoalbuminaemia:0.02), (CANDESARTAN:Hypoalbuminaemia:0.46), (CARFILZO MIB:Hypoalbuminaemia:0.31), (CASPOFUNGIN:Hypoalbuminaemia:0.21), (CERTOLIZUMAB PEGOL:Hypoalbuminaemia:0.15), (CLOPIDOGREL BISULFATE:Hypoalbuminaemia:0.98), (CLOZAPINE:Hypoalbuminaemia:0.08), (DASATINIB: Hypoalbuminaemia:0.7), (DIVALPROEX SODIUM:Hypoalbuminaemia:0.35), (DOCETAXEL:Hypoalbuminaemia:0.1), (DOCUSATE:Hy poalbuminaemia:0.21), (EDOXABAN TOSYLATE:Hypoalbuminaemia:0.02), (EPIRUBICIN:Hypoalbuminaemia:0.06), (FENOFIBRA TE:Hypoalbuminaemia:0.13), (FERROUS SULFATE:Hypoalbuminaemia:0.75), (FLECAINIDE:Hypoalbuminaemia:0.03), (FOSPHENYT OINFOSPHENYTOIN SODIUM:Hypoalbuminaemia:0.46), (GLIPIZIDE:Hypoalbuminaemia:0.1), (IBRUTINIB:Hypoal buminaemia:0.52), (INVESTIGATIONAL PRODUCT:Hypoalbuminaemia:0.11), (LABETALOLLABETALOL HYDROCHLORIDE:Hypoalbuminaemia:0.27), (LIDOCAINEPRILOCAINE:Hypoalbuminaem ia:0.24), (LOVASTATIN:Hypoalbuminaemia:0.29), (METHENOLONE ENANTHATE:Hypoalbuminaemia:0.23), (METOPROLOL TARTRATE:Hypoalbuminaemia:0.1), (NILOTINIB:Hypoalbuminaemia:1.41), (NOREPINEPHR INE:Hypoalbuminaemia:0.24), (OLANZAPINE:Hypoalbuminaemia:0.07), (OXCARBAZEPINE: Hypoalbuminaemia:0.45), (PAZOPANIB HYDROCHLORIDE:Hypoalbuminaemia:0.19), (POSACONAZOLE:Hypoalbuminaemia:0.16), (SEVELAMER HYDROCHLORIDE:Hypoalbuminaemia:0.13), (SOTALOL:Hypoalbuminaemia:0.17), (SULFA METHOXAZOLE:Hypoalbuminaemia:0.55), (THALIDOMIDE:Hypoalbuminaemia:1.19), (THI OPENTAL:Hypoalbuminaemia:0.03), (TOPIRAMATE:Hypoalbuminaemia:0.39), (VALGANCI CLOVIR HYDROCHLORIDE:Hypoalbuminaemia:0.46), (VENLAFAXINE HYDROCHLORIDE:Hypoalbuminaemia:0.34), (.ALPHA.-TOCOPHEROL:Hypocalcaemia:0.16), (.ALPHA.1-PROTEINASE INHIBITOR HUMAN:Hypocalcaemia:0.01), (ACETAMINOPHENCAFFEINE:Hypocalcaemia:0.03), (ALC OHOL:Hypocalcaemia:0.38), (AMISULPRIDE:Hypocalcaemia:0.27), (AMLODIPINEPERIND OPRIL:Hypocalcaemia:0.13), (AMLODIPINEVALSARTAN:Hypocalcaemia:0.2), (ARMODAF INIL:Hypocalcaemia:0.11), (ASPIRINMAGNESIUM OXIDE:Hypocalcaemia:0.28), (ATROPINE SULFATE:Hypocalcaemia:0.07), (BENAZEPRIL HYDROCHLORIDE:Hypocalcaemia:0.68), (BENAZEPRILBENAZEPRIL HYDROCHLORIDE:Hypocalcaemia:0.79), (BENIDIPINE HYDROCHLORIDE:Hypocalcaemia:0.01), (BERAPROST:Hypocalcaemia:0.15), (BERAPROS T SODIUM:Hypocalcaemia:0.68), (BIOTIN:Hypocalcaemia:0.04), (BLINATUMOMAB:Hypocalc aemia:0.39), (BRINZOLAMIDE:Hypocalcaemia:0.16), (BUPRENORPHINE HYDROCHLORIDE:Hypocalcaemia:0.34), (BUPRENORPHINE HYDROCHLORIDENALOXONE HYDROCHLORIDE:Hypocalcaemia:0.05), (BUPROPION:Hypocalcaemia:0.84), (CABOZANTI NIB:Hypocalcaemia:0.29), (CANAGLIFLOZINMETFORMIN HYDROCHLORIDE:Hypocalcaemia:0.22), (CANAKINUMAB:Hypocalcaemia:0.59), (CARVE DILOL PHOSPHATE:Hypocalcaemia:0.81), (CASANTHRANOLDOCUSATE SODIUM:Hypocalcaemia:0.01), (CEFOTAXIME SODIUM:Hypocalcaemia:0.97), (CEFOTAXIMECEFOTAXIME SODIUM:Hypocalcaemia:0.46), (CEFPODOXIME PROXETIL:Hypocalcaemia:0.25), (COLISTIN:Hypocalcaemia:0), (CORTISONEHYDROCOR TISONE:Hypocalcaemia:0.72), (CYPROHEPTADINE:Hypocalcaemia:0.08), (DACLATASVIR: Hypocalcaemia:0.29), (DACLIZUMAB:Hypocalcaemia:0.75), (DANAZOL:Hypocalcaemia:0.89), (DASABUVIROMBITASVIRPARITAPREVIRRITONAVIR:Hypocalcaemia:0.06), (DASAT INIB:Hypocalcaemia:1.99), (DESFLURANE:Hypocalcaemia:0.15), (DIGITOXIN:Hypocalcaemi a:0.73), (DIPYRIDAMOLE:Hypocalcaemia:0.78), (DIPYRONE:Hypocalcaemia:0.3), (DIVALPR OEX SODIUM:Hypocalcaemia:0.22), (DOBUTAMINE HYDROCHLORIDE:Hypocalcaemia:0.67), (DOFETILIDE:Hypocalcaemia:0.4), (DOLASETRO N MESYLATE:Hypocalcaemia:0.34), (DOPAMINEDOPAMINE HYDROCHLORIDE:Hypocalcaemia:0.33), (DRONEDARONE:Hypocalcaemia:0.03), (ECONA ZOLE:Hypocalcaemia:0.6), (EDOXABAN TOSILATE MONOHYDRATE:Hypocalcaemia:0.07), (ELETRIPTAN HYDROBROMIDE:Hypocalcaemia:0.4), (ENALAPRIL MALEATE:Hypocalcaemia:1.45), (EPIRUBICIN:Hypocalcaemia:1.57), (EPIRUBICIN HYDROCHLORIDE:Hypocalcaemia:0.14), (ESMOLOL HYDROCHLORIDE:Hypocalcaemia:0.17), (ESTROGENS, CONJUGATED:Hypocalcaemia:0.31), (ETORICOXIB:Hypocalcaemia:0.36), (ETRETINATE:H ypocalcaemia:0.73), (EVOLOCUMAB:Hypocalcaemia:0.28), (FERROUS GLUCONATE:Hypocalcaemia:0.02), (FERROUS SULFATEGLYCINE:Hypocalcaemia:0.06), (FLUDROCORTISONE ACETATE:Hypocalcaemia:0.34), (FLUOCINONIDE:Hypocalcaemia:0), (FLUTICASONE FUROATE:Hypocalcaemia:0.1), (FLUTICASONE FUROATEVILANTEROL TRIFENATATE:Hypocalcaemia:0.57), (FORMOTEROL:Hypocalcaemia:0.49), (FORMOTERO L FUMARATE DIHYDRATEMOMETASONE FUROATE:Hypocalcaemia:0.32), (GLYCOPYRROLATE:Hypocalcaemia:0.27), (GLYCOPYRR ONIUMINDACATEROL:Hypocalcaemia:0.78), (HERBALSSAW PALMETTO:Hypocalcaemia:0.39), (HERBALSTURMERIC:Hypocalcaemia:0.19), (HYDROC HLOROTHIAZIDETELMISARTAN:Hypocalcaemia:0.31), (HYDROCORTISONE ACETATE:Hypocalcaemia:0.33), (HYDROXYCHLOROQUINE DIPHOSPHATE:Hypocalcaemia:0.02), (IBRITUMOMAB TIUXETAN:Hypocalcaemia:0.2), (ILOPROST:Hypocalcaemia:1.46), (IMIDAPRIL:Hypocalcae mia:0.04), (IMMUNE GLOBULIN NOS:Hypocalcaemia:0.06), (INDACATEROL:Hypocalcaemia:0.18), (INOTUZUMAB OZOGAMICIN:Hypocalcaemia:0.08), (INTERFERON:Hypocalcaemia:0.65), (INTERFERON ALFA-2A:Hypocalcaemia:0.51), (INTERFERON ALFA-2B:Hypocalcaemia:0.67), (IRINOTECAN:Hypocalcaemia:0.37), (IRINOTECAN HYDROCHLORIDE:Hypocalcaemia:1.27), (ISOFLURANE:Hypocalcaemia:0.2), (ISOSORBID E:Hypocalcaemia:0.92), (IXABEPILONE:Hypocalcaemia:0.13), (LABETALOL HYDROCHLORIDE:Hypocalcaemia:0.1), (LANREOTIDE ACETATE:Hypocalcaemia:0.3), (LAPATINIB:Hypocalcaemia:0.01), (LEDIPASVIRSOFOSBU VIR:Hypocalcaemia:1.64), (LERCANIDIPINE HYDROCHLORIDE:Hypocalcaemia:1.11), (LEVALBUTEROL:Hypocalcaemia:0.05), (LEVOL EUCOVORIN CALCIUM:Hypocalcaemia:0.8), (LITHIUM CARBONATE:Hypocalcaemia:0.75), (MECLIZINEMECLIZINE HYDROCHLORIDE:Hypocalcaemia:0.37), (MEPERIDINE HYDROCHLORIDE:Hypocalcaemia:0.46), (METAXALONE:Hypocalcaemia:0.17), (METHAD ONE HYDROCHLORIDE:Hypocalcaemia:1.15), (METHENAMINE HIPPURATE:Hypocalcaemia:0.47), (METHENOLONE ENANTHATE:Hypocalcaemia:0.55), (METHOCARBAMOL:Hypocalcaemia:0.28), (METHYLP REDNISOLONE ACETATE:Hypocalcaemia:0.21), (METILDIGOXIN:Hypocalcaemia:0.35), (MIDODRINE:Hyp ocalcaemia:0.46), (MINOCYCLINEMINOCYCLINE HYDROCHLORIDE:Hypocalcaemia:1.49), (MINODRONIC ACID:Hypocalcaemia:0.1), (MOXONIDINE:Hypocalcaemia:0.38), (NABUMETONE:Hypocalca emia:0.08), (NALOXONE:Hypocalcaemia:0.01), (NALOXONE HYDROCHLORIDETILIDINE HYDROCHLORIDE:Hypocalcaemia:0.48), (NATEGLINIDE:Hypocalcaemia:0.17), (NIACIN:H ypocalcaemia:0.37), (NIRAPARIB:Hypocalcaemia:0.62), (NOREPINEPHRINE:Hypocalcaemia: 0.83), (NORTRIPTYLINE:Hypocalcaemia:0.26), (OXCARBAZEPINE:Hypocalcaemia:0.88), (P ANTOPRAZOLE MAGNESIUM:Hypocalcaemia:0.96), (PENICILLIN G:Hypocalcaemia:0.32), (PENTOSTATIN:Hypocalcaemia:0.32), (PHLOROGLUCINOL\1,3,5-TRIMETHOXYBENZENE:Hypocalcaemia:0.01), (PILSICAINIDE HYDROCHLORIDE:Hypocalcaemia:0.26), (PIMOBENDAN:Hypocalcaemia:0.39), (POTASSIU M CITRATETRISODIUM CITRATE DIHYDRATE:Hypocalcaemia:0.16), (PRAMIPEXOLE DIHYDROCHLORIDE:Hypocalcaemia:0.36), (PREDNISOLONE SODIUM PHOSPHATE:Hypocalcaemia:0.17), (PRIMIDONE:Hypocalcaemia:0.77), (PRISTINAMYCIN: Hypocalcaemia:0.2), (PROBIOTICS NOS:Hypocalcaemia:0.85), (PROGESTERONE:Hypocalcaemia:0.11), (PROPRANOLOLPROP RANOLOL HYDROCHLORIDE:Hypocalcaemia:0.08), (RADIATION THERAPY:Hypocalcaemia:0.78), (RANOLAZINE:Hypocalcaemia:1.27), (RIVASTIGMINE:Hy pocalcaemia:0.13), (RIVASTIGMINE TARTRATE:Hypocalcaemia:0.35), (RIZATRIPTAN BENZOATE:Hypocalcaemia:0.26), (ROFLUMILAST:Hypocalcaemia:0.69), (ROPINIROLE:Hy pocalcaemia:0.13), (SACUBITRILVALSARTAN:Hypocalcaemia:0.56), (SIPULEUCEL-T:Hypocalcaemia:0.48), (SITAGLIPTIN:Hypocalcaemia:1.15), (SUCCINYLCHOLINE CHLORIDE:Hypocalcaemia:0.01), (SUMATRIPTANSUMATRIPTAN SUCCINATE:Hypocalcaemia:0.09), (TEDUGLUTIDEWATER:Hypocalcaemia:0), (TELOTRIS TAT ETHYL:Hypocalcaemia:0), (TERAZOSIN HYDROCHLORIDE:Hypocalcaemia:0.11), (TETRACYCLINE:Hypocalcaemia:0), (THEOPHY LLINE ANHYDROUS:Hypocalcaemia:0.11), (TIOTROPIUM:Hypocalcaemia:0.88), (TIPIRACIL HYDROCHLORIDETRIFLURIDINE:Hypocalcaemia:0.26), (TIZANIDINE HYDROCHLORIDE:Hypocalcaemia:0.34), (TOCOPHEROL:Hypocalcaemia:0.12), (TOPIRAM ATE:Hypocalcaemia:0.91), (TRABECTEDIN:Hypocalcaemia:0.22), (TRANDOLAPRIL:Hypoca lcaemia:0.01), (VARENICLINE TARTRATE:Hypocalcaemia:0.36), (VINORELBINE TARTRATE:Hypocalcaemia:0.81), (VITAMIN A:Hypocalcaemia:0.22), (VITAMIN B:Hypocalcaemia:0.74), (ZINC:Hypocalcaemia:0.1), (ZIV-AFLIBERCEPT:Hypocalcaemia:0.33), (ACETAMINOPHENOXYCODONEOXYCODONE HYDROCHLORIDE:Hypogammaglobulinaemia:0.72), (ACETAMINOPHENPROPOXYPHEN E NAPSYLATE:Hypogammaglobulinaemia:0.31), (AGALSIDASE BETA:Hypogammaglobulinaemia:0.34), (ALBIGLUTIDE:Hypogammaglobulinaemia:0.14), (A MIODARONE HYDROCHLORIDE:Hypogammaglobulinaemia:1.1), (AMLODIPINEVALSARTAN:Hypoga mmaglobulinaemia:0.17), (AMMONIUM LACTATE:Hypogammaglobulinaemia:0.3),(ARMODAFINIL:Hypogammaglobulinaemia:1.86), (BENAZEPRILBENAZEPRIL HYDROCHLORIDE:Hypogammaglobulinaemia:0.82), (BISACODYL OR DOCUSATE SODIUM:Hypogammaglobulinaemia:1.21), (BRIMONIDINE TARTRATETIMOLOL MALEATE:Hypogammaglobulinaemia:0.15), (BRINZOLAMIDE:Hypogammaglobulinaemia:0.8), (BROMAZEPAM:Hypogammaglobulinaemia:0.07), (BUDESONIDEFORMOTEROL FUMARATE:Hypogammaglobulinaemia:0.73), (BUPRENORPHINE HYDROCHLORIDE:Hypogammaglobulinaemia:0.36), (BUPRENORPHINE HYDROCHLORIDENALOXONE HYDROCHLORIDE:Hypogammaglobulinaemia:0.2), (BUTYLSCOPOLAMINE BROMIDE:Hypogammaglobulinaemia:0.19), (CALCIUMMAGNESIUMZINC:Hypogammagl obulinaemia:0.04), (CANAKINUMAB:Hypogammaglobulinaemia:0.92), (CANDESARTAN CILEXETIL:Hypogammaglobulinaemia:1.1), (CANDESARTAN CILEXETILHYDROCHLOROTHIAZIDE:Hypogammaglobulinaemia:0.77), (CAPSAICIN:Hy pogammaglobulinaemia:0.14), (CASPOFUNGIN:Hypogammaglobulinaemia:0.33), (CERTOLIZ UMAB PEGOL:Hypogammaglobulinaemia:1.47), (CLOZAPINE:Hypogammaglobulinaemia:0.39), (CO RTISONEHYDROCORTISONE:Hypogammaglobulinaemia:0.93), (CYPROHEPTADINE HYDROCHLORIDE:Hypogammaglobulinaemia:0.24), (DACLIZUMAB:Hypogammaglobulina emia:1.16), (DALTEPARIN:Hypogammaglobulinaemia:0.18), (DAPAGLIFLOZIN PROPANEDIOL:Hypogammaglobulinaemia:0.08), (DASATINIB:Hypogammaglobulinaemia:1.22), (DEVICE:Hypogammaglobulinaemia:0.26), (DEXAMETHASONE ACETATE:Hypogammaglobulinaemia:0.19), (DEXTROAMPHETAMINE SULFATE:Hypogammaglobulinaemia:0.01), (DIPYRIDAMOLE:Hypogammaglobulinaemia:0.23), (DIPYRONE:Hypogammaglobulinaemia:0.24), (DOXYLAMINE SUCCINATE:Hypogammaglobulinaemia:0.14), (EDOXABAN TOSYLATE:Hypogammaglobulinaemia:0.67), (ELOTUZUMAB:Hypogammaglobulinaemia:0.17), (ESOMEPRAZOLE MAGNESIUMNAPROXEN:Hypogammaglobulinaemia:0.15), (ESTROGENS:Hypogammaglo bulinaemia:0.33), (ESTRONESUS SCROFA OVARY:Hypogammaglobulinaemia:0.09), (EVEROLIMUS:Hypogammaglobulinaemia:0.34), (E VOLOCUMAB:Hypogammaglobulinaemia:0.9), (FENOFIBRIC ACID:Hypogammaglobulinaemia:0.2), (FORMOTEROL:Hypogammaglobulinaemia:0.44), (FOR MOTEROL FUMARATEMOMETASONE FUROATE:Hypogammaglobulinaemia:0.93), (GLYCOPYRROLATE:Hypogammaglobulinaemi a:0.64), (GLYCOPYRRONIUM:Hypogammaglobulinaemia:0.44), (HUMAN C1-ESTERASE INHIBITOR:Hypogammaglobulinaemia:0.04), (HYDROCHLOROTHIAZIDEVALSARTAN:H ypogammaglobulinaemia:0.95), (HYDROCORTISONE ACETATE:Hypogammaglobulinaemia:0.36), (HYDROXYUREA:Hypogammaglobulinaemia:0.03), (IBRITUMOMAB TIUXETAN:Hypogammaglobulinaemia:0.23), (ILOPROST:Hypogammaglobulinaemia:0.64), (I MATINIB MESYLATE:Hypogammaglobulinaemia:0.03), (IMIPENEM:Hypogammaglobulinaemia:0.44), (I NDAPAMIDE:Hypogammaglobulinaemia:0.21), (INDAPAMIDEPERINDOPRIL:Hypogamma globulinaemia:0.22), (INDOMETHACIN:Hypogammaglobulinaemia:0.69), (INFLIXIMAB-DYYB:Hypogammaglobulinaemia:0.08), (INTERFERON GAMMA-1B:Hypogammaglobulinaemia:0.75), (IODINE:Hypogammaglobulinaemia:0.04), (LACTASE:Hy pogammaglobulinaemia:0.56), (LEDIPASVIRSOFOSBUVIR:Hypogammaglobulinaemia:0.34), (LERCANIDIPINE:Hypogammaglobulinaemia:0.62), (LERCANIDIPINE HYDROCHLORIDE:Hypogammaglobulinaemia:0.31), (LINACLOTIDE:Hypogammaglobulina emia:0.44), (LINAGLIPTIN:Hypogammaglobulinaemia:0.68), (LIOTHYRONINE:Hypogammag lobulinaemia:0.21), (LITHIUM:Hypogammaglobulinaemia:0.16), (LOMITAPIDE MESYLATE:Hypogammaglobulinaemia:0.03), (MAGNESIUM GLUCONATE:Hypogammaglobulinaemia:0.11), (MEGESTROL:Hypogammaglobulinaemia:0.01), (MEGESTROL ACETATE:Hypogammaglobulinaemia:0.02), (MEPOLIZUMAB:Hypogammaglobulinaemia:0.7), (METHENAMINE HIPPURATE:Hypogammaglobulinaemia:1.12), (METHIMAZOLE:Hypogammaglobulinaemia:00.68), (METHYLPREDNISOLONE ACETATE:Hypogammaglobulinaemia:1.04), (NADOLOL:Hypogammaglobulinaemia:0.19), (N ALOXONE HYDROCHLORIDEOXYCODONE HYDROCHLORIDE:Hypogammaglobulinaemia:0.79), (NATEGLINIDE:Hypogammaglobulina emia:0.25), (NICOTINE:Hypogammaglobulinaemia:0.59), (NILOTINIB:Hypogammaglobulinae mia:1.44), (NINTEDANIB:Hypogammaglobulinaemia:0.47), (OSELTAMIVIR:Hypogammaglob ulinaemia:0.42), (OXYMETAZOLINE:Hypogammaglobulinaemia:0.11), (PANCRELIPASE:Hyp ogammaglobulinaemia:0.02), (PAROXETINEPAROXETINE HYDROCHLORIDE:Hypogammaglobulinaemia:0.65), (PATIROMER:Hypogammaglobulinaem ia:0.08), (PAZOPANIB HYDROCHLORIDE:Hypogammaglobulinaemia:0.28), (PEGINTERFERON BETA-1A:Hypogammaglobulinaemia:0.56), (PENICILLIN V:Hypogammaglobulinaemia:0.53), (PHENYTOIN:Hypogammaglobulinaemia:0.49), (PHENYT OIN SODIUM:Hypogammaglobulinaemia:0.4), (PONATINIB:Hypogammaglobulinaemia:1.24), (QUI NAPRIL:Hypogammaglobulinaemia:0.08), (RETINOL:Hypogammaglobulinaemia:0.4), (RIVAS TIGMINE TARTRATE:Hypogammaglobulinaemia:0.02), (SACUBITRILVALSARTAN:Hypogammaglob ulinaemia:0.46), (SALMETEROL:Hypogammaglobulinaemia:0.1), (SECUKINUMAB:Hypogam maglobulinaemia:0.57), (SODIUM OXYBATE:Hypogammaglobulinaemia:1.83), (TERAZOSIN HYDROCHLORIDE:Hypogammaglobulinaemia:0.05), (TERIFLUNOMIDE:Hypogammaglobul inaemia:0.8), (THEOPHYLLINE:Hypogammaglobulinaemia:0.23), (TIGECYCLINE:Hypogamm aglobulinaemia:0.12), (TIMOLOL:Hypogammaglobulinaemia:0.85), (TIMOLOL MALEATE:Hypogammaglobulinaemia:0.69), (TINZAPARIN SODIUM:Hypogammaglobulinaemia:0.14), (TOFACITINIB CITRATE:Hypogammaglobulinaemia:0.07), (TOLTERODINE:Hypogammaglobulinaemia:0.01), (TRETINOIN:Hypogammaglobulinaemia:0.08), (VARENICLINE TARTRATE:Hypogammaglobulinaemia:1.55), (VITAMIN A:Hypogammaglobulinaemia:0.71), (ACETAMINOPHENBUTALBITALCAFFEINE:Hypogly caemia:0.22), (ACLIDINIUM BROMIDE:Hypoglycaemia:0.03), (ALEMTUZUMAB:Hypoglycaemia:0), (AMISULPRIDE:Hyp oglycaemia:0.19), (ANASTROZOLE:Hypoglycaemia:0.63),(ARMODAFINIL:Hypoglycaemia:0 0.1), (BROMAZEPAM:Hypoglycaemia:0.04), (BUPRENORPHINE HYDROCHLORIDE:Hypoglycaemia:0.41), (BUPRENORPHINE HYDROCHLORIDENALOXONE HYDROCHLORIDE:Hypoglycaemia:0.03), (BUPROPION:Hypoglycaemia:0.42), (CALCIUM VITAMIN D:Hypoglycaemia:0.38), (CANDESARTAN CILEXETILHYDROCHLOROTHIAZIDE:Hypoglycaemia:0.65), (CARFILZOMIB:Hypoglyca emia:0.42), (CARVEDILOL PHOSPHATE:Hypoglycaemia:0.36), (CEFAZOLIN:Hypoglycaemia:0.61), (CEFOTAXIME SODIUM:Hypoglycaemia:0.02), (CERTOLIZUMAB PEGOL:Hypoglycaemia:0.61), (CIPROFLOXACIN HYDROCHLORIDE:Hypoglycaemia:0.15), (CLINDAMYCINCLINDAMYCIN PHOSPHATE:Hypoglycaemia:1.45), (COLCHICINE:Hypoglycaemia:0.52), (CORTISONEHY DROCORTISONE:Hypoglycaemia:0.18), (DESLORATADINE:Hypoglycaemia:0.22), (DICLOF ENAC:Hypoglycaemia:0.48), (DIVALPROEX SODIUM:Hypoglycaemia:0.93), (DOCETAXEL:Hypoglycaemia:0.25), (DOFETILIDE:Hypogly caemia:0.1), (DULOXETINE:Hypoglycaemia:0.29), (EDOXABAN TOSYLATE:Hypoglycaemia:0.3), (ELETRIPTAN HYDROBROMIDE:Hypoglycaemia:0.37), (EMPAGLIFLOZINLINAGLIPTIN:Hypoglycaemia:0.63), (EMPAGLIFLOZINMETFORMIN HYDROCHLORIDE:Hypoglycaemia:0.04), (EPIRUBICIN:Hypoglycaemia:0.29), (EPLERENO NE:Hypoglycaemia:0.11), (EVOLOCUMAB:Hypoglycaemia:0.59), (EZETIMIBESIMVASTAT IN:Hypoglycaemia:0.05), (FESOTERODINE FUMARATE:Hypoglycaemia:0.11), (FLECAINIDE:Hypoglycaemia:0.6), (FLUTICASONE FUROATEVILANTEROL TRIFENATATE:Hypoglycaemia:0.23), (FORMOTEROL:Hypoglycaemia:0.08), (HYDROCHLO ROTHIAZIDEIRBESARTAN:Hypoglycaemia:0.34), (HYDROCHLOROTHIAZIDELISINOP RIL:Hypoglycaemia:0.69), (HYDROCHLOROTHIAZIDELOSARTAN POTASSIUM:Hypoglycaemia:0.13), (HYDROCHLOROTHIAZIDETELMISARTAN:Hypogly caemia:0.03), (HYDROCHLOROTHIAZIDEVALSARTAN:Hypoglycaemia:0.56), (ILOPROST:Hypoglycaemia:0.46), (IMIPRAMINE HYDROCHLORIDE:Hypoglycaemia:0.07), (INDAPAMIDEPERINDOPRIL:Hypoglycaemia:0.35), (INSULIN DEGLUDEC:Hypoglycaemia:0.31), (IPRATROPIUM:Hypoglycaemia:0.72), (IRINOTECAN:Hy poglycaemia:0.44), (IRINOTECAN HYDROCHLORIDE:Hypoglycaemia:0.14), (ISOSORBIDE:Hypoglycaemia:0.53), (LEVALBUT EROL HYDROCHLORIDE:Hypoglycaemia:0.53), (LEVOLEUCOVORIN CALCIUM:Hypoglycaemia:0.16), (LISDEXAMFETAMINE DIMESYLATE:Hypoglycaemia:0.07), (LITHIUM CARBONATE:Hypoglycaemia:0.89), (METAXALONE:Hypoglycaemia:0.15), (METHENAMI NE HIPPURATE:Hypoglycaemia:0.23), (METHYLPREDNISOLONE ACETATE:Hypoglycaemia:0.04), (MIDODRINE:Hypoglycaemia:0.15), (MOXIFLOXACIN HYDROCHLORIDE:Hypoglycaemia:0.93), (MOXONIDINE:Hypoglycaemia:0.02), (NADOLO L:Hypoglycaemia:0.05), (NEBIVOLOL:Hypoglycaemia:0.47), (NEBIVOLOL HYDROCHLORIDE:Hypoglycaemia:0.25), (NIACIN:Hypoglycaemia:0.25), (NILOTINIB:Hypo glycaemia:1.29), (NITROFURANTOIN:Hypoglycaemia:0.62), (NORTRIPTYLINE:Hypoglycae mia:0.14), (OSELTAMIVIR:Hypoglycaemia:0.16), (PANTOPRAZOLE MAGNESIUM:Hypoglycaemia:0.32), (PENICILLIN:Hypoglycaemia:0.25), (PHENYTOIN SODIUM:Hypoglycaemia:0.48), (PYRAZINAMIDE:Hypoglycaemia:0.67), (PYRIDOXINE:Hyp oglycaemia:0.19), (RABEPRAZOLE:Hypoglycaemia:0.26), (RANOLAZINE:Hypoglycaemia:0.82), (SACUBITRILVALSARTAN:Hypoglycaemia:0.39), (SECUKINUMAB:Hypoglycaemia:0), (SODIUM OXYBATE:Hypoglycaemia:0.24), (SODIUM PHOSPHATE, DIBASICSODIUM PHOSPHATE, MONOBASIC:Hypoglycaemia:0.01), (SUMATRIPTAN:Hypoglycaemia:0.13), (SUMATRIPTA NSUMATRIPTAN SUCCINATE:Hypoglycaemia:0.13), (TAMSULOSIN HYDROCHLORIDE:Hypoglycaemia:0.57), (TEMAZEPAM:Hypoglycaemia:0.62), (TERIFLUN OMIDE:Hypoglycaemia:0.05), (THALIDOMIDE:Hypoglycaemia:1.08), (TIMOLOL:Hypoglyca emia:0.16), (TINZAPARIN SODIUM:Hypoglycaemia:0.18), (TIOTROPIUM:Hypoglycaemia:0.15), (TIZANIDINE HYDROCHLORIDE:Hypoglycaemia:0.72), (TOCILIZUMAB:Hypoglycaemia:0), (UMECLIDIN IUM BROMIDEVILANTEROL TRIFENATATE:Hypoglycaemia:0.05), (VITAMIN B:Hypoglycaemia:0.19), (ABACAVIR SULFATEDOLUTEGRAVIR SODIUMLAMIVUDINE:Hypogonadism:0.76), (ALISKIREN HEMIFUMARATE:Hypogonadism:0.03), (AMIODARONE HYDROCHLORIDE:Hypogonadism:0.68), (BISACODYL:Hypogonadism:0.09), (BISACODYL OR DOCUSATE SODIUM:Hypogonadism:0.73), (BISOPROLOL FUMARATE:Hypogonadism:0.73), (BUDESONIDEFORMOTEROL FUMARATE:Hypogonadism:0.03), (CALCIUM ACETATE:Hypogonadism:0.22), (CANAGLIFLOZINMETFORMIN HYDROCHLORIDE:Hypogonadism:0.08), (CANDESARTAN:Hypogonadism:0.72), (CANDES ARTAN CILEXETIL:Hypogonadism:0.74), (CARVEDILOL PHOSPHATE:Hypogonadism:0.36), (CEPHALEXIN:Hypogonadism:0.92), (CIMETIDINE:Hyp ogonadism:0.01), (CLOTRIMAZOLE:Hypogonadism:0.46), (CLOZAPINE:Hypogonadism:0.38), (DABIGATRAN:Hypogonadism:0.07), (DACLIZUMAB:Hypogonadism:0.15), (DALFAMPRI DINE:Hypogonadism:0.14), (DASATINIB:Hypogonadism:0.38), (DEVICE:Hypogonadism:1.09), (DOFETILIDE:Hypogonadism:0.07), (DOLUTEGRAVIR:Hypogonadism:0.33), (DRONABINO L:Hypogonadism:0.03), (DULAGLUTIDE:Hypogonadism:0.22), (DULOXETINE:Hypogonadis m:0.65), (ELETRIPTAN HYDROBROMIDE:Hypogonadism:0.53), (EMPAGLIFLOZINMETFORMIN HYDROCHLORIDE:Hypogonadism:0.2), (ESTROGENS, CONJUGATED:Hypogonadism:0.45), (ESTROGENS, CONJUGATEDMEDROXYPROGESTERONE ACETATE:Hypogonadism:0.28), (EVEROLIMUS:Hypogonadism:0.31), (FISH OIL\OMEGA-3 FATTY ACIDS:Hypogonadism:0.01), (FLECAINIDE:Hypogonadism:0.42), (GLICLAZIDE:Hypogonadi sm:0.21), (HYDROCHLOROTHIAZIDEIRBESARTAN:Hypogonadism:0.25), (HYOSCYAMI NE:Hypogonadism:0.23), (IBRUTINIB:Hypogonadism:1.16), (INDOMETHACIN:Hypogonadis m:0.27), (LANREOTIDE ACETATE:Hypogonadism:0.49), (LERCANIDIPINE HYDROCHLORIDE:Hypogonadism:0.33), (LEVALBUTEROL HYDROCHLORIDE:Hypogonadism:0.32), (LIDOCAINE HYDROCHLORIDE:Hypogonadism:0), (MOXIFLOXACIN HYDROCHLORIDE:Hypogonadism:0.49), (NATEGLINIDE:Hypogonadism:0.03), (NIFEDIPIN E:Hypogonadism:0.56), (NILOTINIB:Hypogonadism:0.4), (NITROFURANTOINNITROFURA NTOIN MONOHYDRATE:Hypogonadism:0.05), (NORTRIPTYLINE:Hypogonadism:0.37), (PANTOPR AZOLE:Hypogonadism:1.39), (PANTOPRAZOLE MAGNESIUM:Hypogonadism:0.19), (PENICILLIN:Hypogonadism:0.31), (PRIMIDONE:Hypog onadism:0.43), (PYRIDOXINE:Hypogonadism:0.09), (RALOXIFENE HYDROCHLORIDE:Hypogonadism:0.09), (RIZATRIPTAN BENZOATE:Hypogonadism:0.25), (ROFLUMILAST:Hypogonadism:0.06), (SECUKINUMAB: Hypogonadism:0.49), (SOLIFENACIN SUCCINATE:Hypogonadism:0.02), (SUCRALFATE:Hypogonadism:0.25), (SUMATRIPTAN:H ypogonadism:0.26), (SUMATRIPTANSUMATRIPTAN SUCCINATE:Hypogonadism:0.75), (TERIFLUNOMIDE:Hypogonadism:0.45), (TETRACYCLI NE:Hypogonadism:0.4), (THALIDOMIDE:Hypogonadism:0.04), (TOCILIZUMAB:Hypogonadi sm:0.42), (TRIAMCINOLONE:Hypogonadism:0.11), (UMECLIDINIUM BROMIDEVILANTEROL TRIFENATATE:Hypogonadism:0.64), (.ALPHA.1-PROTEINASE INHIBITOR HUMAN:Hypokalaemia:0.93), (ABOBOTULINUMTOXINA:Hypokalaemia:0.37), (ACETAMI NOPHENOXYCODONE HYDROCHLORIDEOXYCODONE TEREPHTHALATE:Hypokalaemia:1.03), (ACETAMINOPHENPROPOXYPHENE HYDROCHLORIDE:Hypokalaemia:0.98), (ACETAMINOPHENPROPOXYPHENE NAPSYLATE:Hypokalaemia:0.68), (ACITRETIN:Hypokalaemia:0.55), (ADENINE:Hypokalae mia:0.07), (ALDESLEUKIN:Hypokalaemia:1.11), (ALECTINIB:Hypokalaemia:0.18), (ALEMTU ZUMAB:Hypokalaemia:1.1), (ALGLUCOSIDASE ALFA:Hypokalaemia:0.22), (AMISULPRIDE:Hypokalaemia:1.22), (AMLODIPINE BESYLATEHYDROCHLOROTHIAZIDEOLMESARTAN MEDOXOMIL:Hypokalaemia:0.53), (AMLODIPINEPERINDOPRIL:Hypokalaemia:1.19), (AMLODIPINEVALSARTAN: Hypokalaemia:1.27), (AMMONIUM GLYCYRRHIZATECYSTEINE HYDROCHLORIDEGLYCINE:Hypokalaemia:0.67), (AMOXAPINE:Hypokalaemia:0.33), (A MOXICILLINCLARITHROMYCINESOMEPRAZOLE:Hypokalaemia:0.76), (AMPHOTERI CIN BDIMYRISTOYLPHOSPHATIDYLCHOLINE, DL-DIMYRISTOYLPHOSPHATIDYLGLYCEROL, DL-:Hypokalaemia:0.21), (ARSENIC TRIOXIDE:Hypokalaemia:0.45), (ASCORBIC ACIDBIOTINCALCIUM PANTOTHENATECOBALAMINFOLIC ACIDNIACINAMIDEPYRIDOXINE HYDROCHLORIDERIBOFLAVINTHIAMINE MONONITRATE:Hypokalaemia:0.33), (ASCORBIC ACIDFERROUS SULFATE:Hypokalaemia:0.02), (ASPIRINCLOPIDOGREL:Hypokalaemia:0.82), (ATRACURI UM BESYLATE:Hypokalaemia:0.7), (AZELASTINE HYDROCHLORIDEFLUTICASONE PROPIONATE:Hypokalaemia:0.05), (BALSALAZIDE DISODIUM:Hypokalaemia:0.51), (BELATACEPT:Hypokalaemia:1.09), (BEPRIDIL:Hypokalae mia:0.87), (BERAPROST:Hypokalaemia:0.29), (BETAMETHASONE DIPROPIONATECALCIPOTRIENE:Hypokalaemia:0.43), (BETAMETHASONE DIPROPIONATECALCIPOTRIENE HYDRATE:Hypokalaemia:0.01), (BETAMETHASONE DIPROPIONATECALCIPOTRIENECALCIPOTRIENE HYDRATE:Hypokalaemia:0.27), (BETAMETHASONECALCIPOTRIENE:Hypokalaemia:0.69), (BETAMIPRONPANIPENEM:Hypokalaemia:1.02), (BIFIDOBACTERIUM LONGUM SUBSP. INFANTIS:Hypokalaemia:0.75), (BILASTINE:Hypokalaemia:1.11), (BLESELUMAB:Hypokala emia:0.31), (BLINATUMOMAB:Hypokalaemia:2.3), (BOSUTINIB:Hypokalaemia:0.42), (BUCI LLAMINE:Hypokalaemia:0.7), (BUPRENORPHINE HYDROCHLORIDENALOXONE HYDROCHLORIDE:Hypokalaemia:0.68), (CABOZANTINIB:Hypokalaemia:0.19), (CALCIPO TRIENE:Hypokalaemia:0.56), (CALCIUM CARBONATEERGOCALCIFEROLRETINOL:Hypokalaemia:0.93), (CALCIUMMAGNESI UMZINC:Hypokalaemia:0.31), (CAPSAICIN:Hypokalaemia:0.1), (CARTEOLOL:Hypokalaemi a:0.25), (CARVEDILOL PHOSPHATE:Hypokalaemia:2.07), (CEFDITOREN PIVOXIL:Hypokalaemia:0.53), (CEFPODOXIME PROXETIL:Hypokalaemia:1.42), (CEFPROZIL:Hypokalaemia:0.28), (CEFTAZIDIME SODIUM:Hypokalaemia:0.1), (CEPHARANTHINE:Hypokalaemia:0.24), (CERTOPARIN SODIUM:Hypokalaemia:0.81), (CEVIMELINE HYDROCHLORIDE:Hypokalaemia:0.04), (CHONDROITIN SULFATE (BOVINE)GLUCOSAMINE HYDROCHLORIDE:Hypokalaemia:0.89), (CLADRIBINE:Hypokalaemia:0.6), (CLENBUTERO L HYDROCHLORIDE:Hypokalaemia:0.28), (CLODRONATE DISODIUM:Hypokalaemia:0.44), (CLOXACILLIN SODIUM:Hypokalaemia:0.53), (COBICISTATELVITEGRAVIREMTRICITABINETENOFO VIR ALAFENAMIDE FUMARATE:Hypokalaemia:0.61), (COBICISTATELVITEGRAVIREMTRICITABINETEN OFOVIR DISOPROXIL FUMARATE:Hypokalaemia:0.23), (COD LIVER OIL:Hypokalaemia:0.68), (COLISTIMETHATE SODIUM:Hypokalaemia:1), (CYCLOPHOSPHAMIDECYTARABINEDEXAMETHASONE DOXORUBICINMETHOTREXATEVINCRISTINE:Hypokalaemia:0.3), (DABIGATRAN ETEXILATE:Hypokalaemia:0.3), (DACLIZUMAB:Hypokalaemia:2.24), (DANAZOL:Hypokala emia:1.76), (DANTROLENE SODIUM:Hypokalaemia:0.9), (DAPAGLIFLOZIN PROPANEDIOLMETFORMIN HYDROCHLORIDE:Hypokalaemia:0.14), (DAUNORUBICIN HYDROCHLORIDE:Hypokalaemia:0.93), (DEFLAZACORT:Hypokalaemia:1.94), (DEGARELI X:Hypokalaemia:0.91), (DESFLURANE:Hypokalaemia:0.67), (DESMOPRESSIN:Hypokalaemia:0.91), (DEVICE:Hypokalaemia:0.63), (DEXTROAMPHETAMINE SULFATE:Hypokalaemia:0.28), (DICLOFENAC SODIUMMISOPROSTOL:Hypokalaemia:0.12), (DIPHENIDOL HYDROCHLORIDE:Hypokalaemia:0.29), (DOLASETRON MESYLATE:Hypokalaemia:0.97), (DOXYLAMINE SUCCINATE:Hypokalaemia:0.66), (DUTASTERIDETAMSULOSIN HYDROCHLORIDE:Hypokalaemia:0.92), (ELBASVIRGRAZOPREVIR:Hypokalaemia:0.43), (EMPAGLIFLOZINLINAGLIPTIN:Hypokalaemia:1.03), (ENALAPRIL MALEATEHYDROCHLOROTHIAZIDE:Hypokalaemia:0.9), (EPACADOSTAT:Hypokalaemi a:0.16), (EPHEDRINE:Hypokalaemia:0.41), (EPHEDRINE HYDROCHLORIDE:Hypokalaemia:0.5), (EPIRUBICIN HYDROCHLORIDE:Hypokalaemia:0.46), (ERIBULIN:Hypokalaemia:1.16), (ESTRONESUS SCROFA OVARY:Hypokalaemia:0.75), (ETHACRYNIC ACID:Hypokalaemia:0.37), (ETHINYL ESTRADIOLNORGESTREL:Hypokalaemia:0.08), (ETHIODIZED OIL:Hypokalaemia:0.08), (ETHYL ICOSAPENTATEICOSAPENT:Hypokalaemia:0.02), (ETRETINATE:Hypokalaemia:1.36), (EV OLOCUMAB:Hypokalaemia:1.61), (FAROPENEM SODIUM:Hypokalaemia:0.25), (FLUCYTOSINE:Hypokalaemia:0.43), (FLUOROURACILIRIN OTECANLEUCOVORIN:Hypokalaemia:0.12), (FLURAZEPAMFLURAZEPAM HYDROCHLORIDE:Hypokalaemia:0.14), (FLUTICASONE PROPIONATEFORMOTEROL FUMARATE:Hypokalaemia:0.21), (FLUVOXAMINE MALEATE:Hypokalaemia:1), (FORMOTEROL FUMARATE DIHYDRATE:Hypokalaemia:0.1), (FORMOTEROL FUMARATEMOMETASONE FUROATE:Hypokalaemia:1.01), (GARENOXACIN:Hypokalaemia:0.25), (GUSPERIMUS TRIHYDROCHLORIDE:Hypokalaemia:0.62), (HERBALSSAW PALMETTO:Hypokalaemia:1.45), (HYDROCHLOROTHIAZIDEMETOPROLOL SUCCINATE:Hypokalaemia:0.86), (HYDROCHLOROTHIAZIDESPIRONOLACTONE:Hypo kalaemia:0.21), (HYDROCORTISONE HEMISUCCINATE:Hypokalaemia:0.33), (HYDROXOCOBALAMINPYRIDOXINE HYDROCHLORIDETHIAMINE HYDROCHLORIDE:Hypokalaemia:0.01), (HYDROXYCHLOROQUINE DIPHOSPHATE:Hypokalaemia:0.74), (IBRITUMOMAB TIUXETAN:Hypokalaemia:2.03), (ICOSAPENT:Hypokalaemia:0.01), (ICOSAPENT ETHYL:Hypokalaemia:0.54), (IGURATIMOD:Hypokalaemia:0.18), (IMIGLUCERASE:Hypoka laemia:0.52), (INFLIXIMAB-DYYB:Hypokalaemia:0.29), (INTERFERON:Hypokalaemia:1.75), (INTERFERON ALFA-2A:Hypokalaemia:1.71), (INTERFERON ALFA-2A OR INTERFERON ALFA-2B:Hypokalaemia:0.3), (INTERFERON ALFA-2B:Hypokalaemia:1.32), (INTERFERON GAMMA-1B:Hypokalaemia:1.08), (ISOFLURANE:Hypokalaemia:0.92), (IXABEPILONE:Hypokalaemia: 0.2), (LACTASE:Hypokalaemia:0.82), (LACTITOL:Hypokalaemia:0.46), (LACTOBACILLUS ACIDOPHILUSLACTOBACILLUS DELBRUECKII SUBSP.

BULGARICUS:Hypokalaemia:0.49), (LACTOBACILLUS RHAMNOSUS:Hypokalaemia:1.21), (LAPATINIB DITOSYLATE:Hypokalaemia:0.95), (LEVOSIMENDAN:Hypokalaemia:0.34), (LEVOTHYRO XINE SODIUMPOTASSIUM IODIDE:Hypokalaemia:0.71), (LISDEXAMFETAMINE DIMESYLATE:Hypokalaemia:0.53), (LITHIUM:Hypokalaemia:0.61), (LOMUSTINE:Hypokala emia:0.49), (LUTETIUM LU-177:Hypokalaemia:0.16), (LYSOZYME HYDROCHLORIDE:Hypokalaemia:0.16), (MAGNESIUM PIDOLATE:Hypokalaemia:0.34), (MAXACALCITOL:Hypokalaemia:0.22), (METFORMIN HYDROCHLORIDESAXAGLIPTIN:Hypokalaemia:0.4), (METFORMIN HYDROCHLORIDESAXAGLIPTIN HYDROCHLORIDE:Hypokalaemia:0.36), (METFORMINSITAGLIPTIN:Hypokalaemia:0.44), (METHENAMINE HIPPURATE:Hypokalaemia:2.02), (METHOXY POLYETHYLENE GLYCOL-EPOETIN BETA:Hypokalaemia:0.5), (METYRAPONE:Hypokalaemia:0.08), (MEXILETINE:Hypokalaemi a:0.14), (MIGLUSTAT:Hypokalaemia:0.69), (MILRINONE LACTATE:Hypokalaemia:0.08), (MOEXIPRIL HYDROCHLORIDE:Hypokalaemia:0.2), (MOGAMULIZUMAB:Hypokalaemia:0.47), (NALOX ONE HYDROCHLORIDETILIDINE HYDROCHLORIDE:Hypokalaemia:2.44), (NATEGLINIDE:Hypokalaemia:1.35), (NORETHIN DRONE:Hypokalaemia:0.5), (NORTRIPTYLINE HYDROCHLORIDE:Hypokalaemia:0.54), (OPIPRAMOL:Hypokalaemia:0.19), (PARATHYROI D HORMONE:Hypokalaemia:0.31), (PARICALCITOL:Hypokalaemia:1.07), (PAZUFLOXACIN: Hypokalaemia:0.12), (PAZUFLOXACIN MESILATE:Hypokalaemia:0.29), (PEGINTERFERON BETA-1A:Hypokalaemia:1.2), (PEGVISOMANT:Hypokalaemia:0.24), (PEGZEREPOETIN ALFA:Hypokalaemia:0.28), (PENICILLIN V BENZATHINE:Hypokalaemia:0.24), (PENTOSTATIN:Hypokalaemia:1.95), (PHENOBARBITA L SODIUM:Hypokalaemia:0.39), (PIRFENIDONE:Hypokalaemia:0.42), (PORFIMER SODIUM:Hypokalaemia:0.73), (PRASUGREL:Hypokalaemia:0.23), (PREDNISOLONE SODIUM PHOSPHATE:Hypokalaemia:1.18), (PROGESTERONE:Hypokalaemia:0.4), (PROPAFENONE HYDROCHLORIDE:Hypokalaemia:0.67), (PYGEUM:Hypokalaemia:0.1), (PYRIMETHAMINE:Hypokalaemia:0.32), (QUINAPRILQUINAPRIL HYDROCHLORIDE:Hypokalaemia:0.45), (RADIATION THERAPY:Hypokalaemia:1.95), (RANIMUSTINE:Hypokalaemia:0.48), (RIFABUTIN:Hypokal aemia:0.08), (RILPIVIRINE HYDROCHLORIDE:Hypokalaemia:0.44), (RIZATRIPTAN:Hypokalaemia:0.43), (RUCAPARI B CAMSYLATE:Hypokalaemia:0.22), (SALMETEROL XINAFOATE:Hypokalaemia:0.72), (SARGRAMOSTIM:Hypokalaemia:0.06), (SARPOGRELA TE HYDROCHLORIDE:Hypokalaemia:0.08), (SCOPOLAMINE HYDROBROMIDE:Hypokalaemia:0.13), (SELEGILINE:Hypokalaemia:0.14), (SELEXIPAG:Hy pokalaemia:0.93), (SIVELESTAT SODIUM:Hypokalaemia:0.16), (SOFOSBUVIRVELPATASVIR:Hypokalaemia:0.23), (SOMAT OSTATIN:Hypokalaemia:0.23), (SPIRAMYCIN:Hypokalaemia:0.49), (STARCH, WHEAT:Hypokalaemia:0.43), (SUPLATAST TOSILATE:Hypokalaemia:0.13), (TABALUMAB:Hypokalaemia:0.08), (TAMOXIFEN CITRATE:Hypokalaemia:0.8), (TEDUGLUTIDE:Hypokalaemia:0.35), (TEDUGLUTIDEWAT ER:Hypokalaemia:1.1), (TEGAFURURACIL:Hypokalaemia:0), (TELOTRISTAT ETHYL:Hypokalaemia:0.7), (TESTOSTERONE UNDECANOATE:Hypokalaemia:1.43), (TETRACYCLINE:Hypokalaemia:1.45), (THIOPENTA L:Hypokalaemia:1.03), (TIANEPTINE:Hypokalaemia:0.29), (TICLOPIDINE HYDROCHLORIDE:Hypokalaemia:0.14), (TIMOLOL MALEATETRAVOPROST:Hypokalaemia:0.1), (TIOTROPIUM:Hypokalaemia:2.13), (TISAGE NLECLEUCEL:Hypokalaemia:0.41), (TOCOPHERYL NICOTINATE, D-.ALPHA.:Hypokalaemia:0.05), (TOSUFLOXACIN TOSYLATE:Hypokalaemia:0.36), (TRIHEXYPHENIDYL:Hypokalaemia:0.4), (UMECLIDINIU M BROMIDE:Hypokalaemia:0.96), (VARDENAFIL HYDROCHLORIDE TRIHYDRATE:Hypokalaemia:0.38), (YEAST:Hypokalaemia:0.02), (ZAFIRLUKAST:Hypokala emia:0.6), (ZALTOPROFEN:Hypokalaemia:0.13), (ZILEUTON:Hypokalaemia:0.42), (ZIV-AFLIBERCEPT:Hypokalaemia:0.83), (UNSPECIFIED INGREDIENT:Hypokalaemic syndrome:0.37), (ACETAMINOPHENOXYCODONEOXYCODONE HYDROCHLORIDE:Hypomagnesaemia:0.56), (ALFUZOSIN HYDROCHLORIDE:Hypomagnesaemia:0.06), (AMISULPRIDE:Hypomagnesaemia:0.44), (AMITRIPTYLINE HYDROCHLORIDE:Hypomagnesaemia:0.46), (AMLODIPINE BESYLATEOLMESARTAN MEDOXOMIL:Hypomagnesaemia:0.06), (AMLODIPINEPERINDOPRIL:Hypomagnesaemia:0.05), (AMLODIPINEVALSARTAN:Hypomagnesaemia:0.23), (ANASTROZOLE:Hypomagnesa emia:1.53), (ANIDULAFUNGIN:Hypomagnesaemia:0.03),(ARMODAFINIL:Hypomagnesaemi a:0.33), (ASPARAGINASE:Hypomagnesaemia:0.14), (ATRACURIUM BESYLATE:Hypomagnesaemia:0.17), (ATROPINE:Hypomagnesaemia:0.14), (BENAZEPRILB ENAZEPRIL HYDROCHLORIDE:Hypomagnesaemia:0.58), (BUDESONIDEFORMOTEROL FUMARATE:Hypomagnesaemia:0.23), (BUPRENORPHINE HYDROCHLORIDE:Hypomagnesaemia:0.44), (BUPRENORPHINE HYDROCHLORIDENALOXONE HYDROCHLORIDE:Hypomagnesaemia:0.23), (CALCITONIN SALMON:Hypomagnesaemia:0.29), (CANAKINUMAB:Hypomagnesaemia:0.02), (CAPTOPRI L:Hypomagnesaemia:0.38), (CARVEDILOL PHOSPHATE:Hypomagnesaemia:1.03), (CEFOTAXIME SODIUM:Hypomagnesaemia:1.18), (CEFOTAXIMECEFOTAXIME SODIUM:Hypomagnesaemia:0.01), (CEFUROXIME AXETIL:Hypomagnesaemia:0.05), (CERITINIB:Hypomagnesaemia:0.16), (CERTOLIZUMAB PEGOL:Hypomagnesaemia:0.95), (CHLORAMBUCIL:Hypomagnesaemia:0.76), (CICLESONID E:Hypomagnesaemia:0.04), (CLINDAMYCIN HYDROCHLORIDE:Hypomagnesaemia:0.05), (CLOZAPINE:Hypomagnesaemia:0.86), (COLIS TIN:Hypomagnesaemia:0.42), (CORTISONEHYDROCORTISONE:Hypomagnesaemia:0.39), (CRIZOTINIB:Hypomagnesaemia:1.2), (CYPROHEPTADINE:Hypomagnesaemia:0.48), (DABR AFENIB:Hypomagnesaemia:0.49), (DACLIZUMAB:Hypomagnesaemia:0.28), (DANAZOL:Hyp omagnesaemia:0.35), (DESFLURANE:Hypomagnesaemia:0.48), (DESLORATADINE:Hypomag nesaemia:0.41), (DESONIDE:Hypomagnesaemia:0.12), (DIGITOXIN:Hypomagnesaemia:0.35), (DIPYRONE:Hypomagnesaemia:0.12), (DOBUTAMINE HYDROCHLORIDE:Hypomagnesaemia:0.36), (DOCUSATE CALCIUM:Hypomagnesaemia:0.48), (DOLASETRON MESYLATE:Hypomagnesaemia:0.25), (DOXEPIN:Hypomagnesaemia:0.31), (ECONAZOLE:H ypomagnesaemia:0.59), (EDOXABAN TOSYLATE:Hypomagnesaemia:0.55), (ELETRIPTAN HYDROBROMIDE:Hypomagnesaemia:0.13), (ELOTUZUMAB:Hypomagnesaemia:0.67), (EMP AGLIFLOZINLINAGLIPTIN:Hypomagnesaemia:0.19), (EVOLOCUMAB:Hypomagnesaemia: 0.3), (EXEMESTANE:Hypomagnesaemia:0.46), (FENOTEROL HYDROBROMIDEIPRATROPIUM BROMIDE:Hypomagnesaemia:0.17), (FESOTERODINE FUMARATE:Hypomagnesaemia:0.66), (FLUINDIONE:Hypomagnesaemia:0.26), (FORMOTER OL:Hypomagnesaemia:0.65), (FORMOTEROL FUMARATE:Hypomagnesaemia:0.66), (FORMOTEROL FUMARATE DIHYDRATEMOMETASONE FUROATE:Hypomagnesaemia:0.71), (GEFITINIB:Hypomagnesaemia:0.17), (GIMERACILOT ERACILTEGAFUR:Hypomagnesaemia:0.45), (GLYCOPYRRONIUMINDACATEROL:Hypo magnesaemia:0.78), (HEPARIN CALCIUM:Hypomagnesaemia:0.11), (HERBALSTURMERIC:Hypomagnesaemia:0.03), (HYD ROCHLOROTHIAZIDETELMISARTAN:Hypomagnesaemia:0.11), (IBRITUMOMAB TIUXETAN:Hypomagnesaemia:0.35), (IDARUBICIN:Hypomagnesaemia:0.08), (ILOPROST:H ypomagnesaemia:0.73), (IMIPRAMINE HYDROCHLORIDE:Hypomagnesaemia:0.26), (INTERFERON ALFA-2A:Hypomagnesaemia:0.05), (INTERFERON ALFA-2B:Hypomagnesaemia:0.3), (INTERFERON GAMMA-1B:Hypomagnesaemia:0.43), (ISOFLURANE:Hypomagnesaemia:0.86), (KETOROLACKETOR OLAC TROMETHAMINE:Hypomagnesaemia:0.22), (LACTOBACILLUS RHAMNOSUS:Hypomagnesaemia:0.63), (LEDIPASVIRSOFOSBUVIR:Hypomagnesaemia:1.12), (LERCANIDIPINE:Hypomagnesaemia:1.76), (LERCANIDIPINE HYDROCHLORIDE:Hypomagnesaemia:0.69), (LEVALBUTEROL HYDROCHLORIDE:Hypomagnesaemia:1.4), (LEVOLEUCOVORIN CALCIUM:Hypomagnesaemia:0.62), (LISDEXAMFETAMINE DIMESYLATE:Hypomagnesaemia:0.14), (LITHIUM CARBONATE:Hypomagnesaemia:0.67), (METAXALONE:Hypomagnesaemia:0.78), (METHE NAMINE HIPPURATE:Hypomagnesaemia:0.72), (METHENOLONE ENANTHATE:Hypomagnesaemia:0.03), (METHIMAZOLE:Hypomagnesaemia:0.16), (METHY LPREDNISOLONE ACETATE:Hypomagnesaemia:0.04), (MINOCYCLINE HYDROCHLORIDE:Hypomagnesaemia:0.21), (NABUMETONE:Hypomagnesaemia:0.11), (NI CARDIPINE:Hypomagnesaemia:0.51), (OMEPRAZOLE SODIUM:Hypomagnesaemia:0.41), (OXCARBAZEPINE:Hypomagnesaemia:1.31), (PARICAL CITOL:Hypomagnesaemia:0.12), (PAROXETINEPAROXETINE HYDROCHLORIDE:Hypomagnesaemia:0.64), (PAZOPANIB HYDROCHLORIDE:Hypomagnesaemia:0.83), (PENICILLIN V:Hypomagnesaemia:0.34), (PENTOSTATIN:Hypomagnesaemia:0.51), (PHENYTOIN SODIUM:Hypomagnesaemia:0.15), (PRAMIPEXOLE:Hypomagnesaemia:0.31), (RADIATION THERAPY:Hypomagnesaemia:0.63), (RALOXIFENE HYDROCHLORIDE:Hypomagnesaemia:0.17), (RAMUCIRUMAB:Hypomagnesaemia:0.44), (R ISEDRONATE SODIUM:Hypomagnesaemia:0.44), (ROFLUMILAST:Hypomagnesaemia:0.81), (ROPIVACAIN E:Hypomagnesaemia:0.29), (SACUBITRILVALSARTAN:Hypomagnesaemia:0.45), (SODIUM OXYBATE:Hypomagnesaemia:0.18), (SODIUM PHOSPHATE, DIBASICSODIUM PHOSPHATE, MONOBASIC:Hypomagnesaemia:0.37), (SUFENTANIL CITRATE:Hypomagnesaemia:0.15), (SUGAMMADEX:Hypomagnesaemia:0.08), (SUMATRIPT ANSUMATRIPTAN SUCCINATE:Hypomagnesaemia:0.53), (TEDUGLUTIDEWATER:Hypomagnesaemia:0.38), (T ERBINAFINE HYDROCHLORIDE:Hypomagnesaemia:0.08), (TERBUTALINE:Hypomagnesaemia:0.48), (TE RBUTALINE SULFATE:Hypomagnesaemia:0.62), (THIOPENTAL:Hypomagnesaemia:0.06), (THYROID, PORCINE:Hypomagnesaemia:0), (TICAGRELOR:Hypomagnesaemia:0.08), (TOCOPHEROL:H ypomagnesaemia:0.08), (TRAMETINIB:Hypomagnesaemia:0.49), (UMECLIDINIUM BROMIDEVILANTEROL TRIFENATATE:Hypomagnesaemia:0.48), (VITAMIN A:Hypomagnesaemia:0.19), (ZINC:Hypomagnesaemia:0.12), (ZIV-AFLIBERCEPT:Hypomagnesaemia:0.01), (ACARBOSE:Hyponatraemia:0.06), (ACETAMINOP HENCODEINE PHOSPHATE:Hyponatraemia:1.12), (ADO-TRASTUZUMAB EMTANSINE:Hyponatraemia:0.21), (ALCOHOL:Hyponatraemia:0.14), (ALENDRONIC ACID:Hyponatraemia:0.49), (AMISULPRIDE:Hyponatraemia:0.26), (AMPHETAMINE ASPARTATEAMPHETAMINE SULFATEDEXTROAMPHETAMINE SACCHARATEDEXTROAMPHETAMINE SULFATE:Hyponatraemia:0.35),(ARMODAFINIL:Hyponatraemia:0.09), (ASPIRIN LYSINE:Hyponatraemia:0.23), (BUDESONIDEFORMOTEROL FUMARATE:Hyponatraemia:0.08), (CALCITONIN SALMON:Hyponatraemia:0.02), (CALCIUM CITRATE:Hyponatraemia:0.06), (CANAKINUMAB:Hyponatraemia:0.04), (CANDESARTAN: Hyponatraemia:1.38), (CARFILZOMIB:Hyponatraemia:0.61), (CARVEDILOL PHOSPHATE:Hyponatraemia:0.26), (CEFAZOLIN SODIUM:Hyponatraemia:0.05), (CIMETIDINE:Hyponatraemia:0.34), (CIPROFLOXACIN HYDROCHLORIDE:Hyponatraemia:0.64), (CLOTRIMAZOLE:Hyponatraemia:0.47), (CORTIS ONEHYDROCORTISONE:Hyponatraemia:0.28), (CYCLOBENZAPRINE:Hyponatraemia:0.42), (CYCLOBENZAPRINE HYDROCHLORIDE:Hyponatraemia:0.35), (DABRAFENIB:Hyponatraemia:0.04), (DESLORA TADINE:Hyponatraemia:0.19), (DOXAZOSIN MESYLATE:Hyponatraemia:0.3), (DUTASTERIDE:Hyponatraemia:0.43), (EDOXABAN TOSYLATE:Hyponatraemia:0.21), (ELETRIPTAN HYDROBROMIDE:Hyponatraemia:0.03), (EPIRUBICIN:Hyponatraemia:0.21), (ERYTHROMY CIN:Hyponatraemia:0.1), (ETORICOXIB:Hyponatraemia:0.23), (FLUTICASONE:Hyponatraemi a:0.32), (FLUTICASONEFLUTICASONE PROPIONATE:Hyponatraemia:0.1), (FORMOTEROL:Hyponatraemia:0.22), (FORMOTEROL FUMARATE:Hyponatraemia:0.17), (GLYCOPYRRONIUMINDACATEROL:Hyponatraemia:0 0.23), (HYDROCHLOROTHIAZIDEIRBESARTAN:Hyponatraemia:0.13), (HYDROXYCHLOR OQUINE DIPHOSPHATE:Hyponatraemia:0.03), (IDELALISIB:Hyponatraemia:0.05), (ILOPROST:Hypon atraemia:0.39), (IMATINIB MESYLATE:Hyponatraemia:0.63), (IMIPENEM:Hyponatraemia:0.18), (IMIPRAMINE HYDROCHLORIDE:Hyponatraemia:0.43), (INDAPAMIDE:Hyponatraemia:0.24), (INDAPAMI DEPERINDOPRIL:Hyponatraemia:0.63), (IRINOTECAN:Hyponatraemia:0.45), (IRINOTECA N HYDROCHLORIDE:Hyponatraemia:0.5), (LABETALOLLABETALOL HYDROCHLORIDE:Hyponatraemia:0.9), (LEDIPASVIRSOFOSBUVIR:Hyponatraemia:0.07), (LERCANIDIPINE:Hyponatraemia:0.94), (LEVOLEUCOVORIN CALCIUM:Hyponatraemia:0.37), (LITHIUM CARBONATE:Hyponatraemia:0.45), (METAXALONE:Hyponatraemia:0.24), (METHADONE HYDROCHLORIDE:Hyponatraemia:0.67), (MIDODRINE:Hyponatraemia:0.27), (MOXIFLOX ACIN:Hyponatraemia:0.35), (NINTEDANIB:Hyponatraemia:0.18), (NORTRIPTYLINE:Hypona traemia:0.07), (PANTOPRAZOLE MAGNESIUM:Hyponatraemia:0.59), (PAROXETINE:Hyponatraemia:0.55), (PAZOPANIB HYDROCHLORIDE:Hyponatraemia:0.25), (PONATINIB:Hyponatraemia:0.51), (POSACONAZ OLE:Hyponatraemia:0.38), (PRAMIPEXOLE:Hyponatraemia:0.42), (PRAMIPEXOLE DIHYDROCHLORIDE:Hyponatraemia:0.28), (PRAVASTATIN SODIUM:Hyponatraemia:1.19), (REGORAFENIB:Hyponatraemia:0.47), (ROFLUMILAST:Hyp onatraemia:0.38), (ROPINIROLE HYDROCHLORIDE:Hyponatraemia:0.2), (SCOPOLAMINE:Hyponatraemia:0.43), (SITAGLIP TIN:Hyponatraemia:0.15), (SUMATRIPTAN:Hyponatraemia:0), (TIGECYCLINE:Hyponatraemi a:0.05), (TIMOLOL:Hyponatraemia:0.24), (TINZAPARIN SODIUM:Hyponatraemia:0.44), (TIOTROPIUM:Hyponatraemia:0.4), (TOCILIZUMAB:Hypona traemia:0.21), (TOFACITINIB CITRATE:Hyponatraemia:0.34), (VERAPAMIL HYDROCHLORIDE:Hyponatraemia:0.99), (VITAMIN B:Hyponatraemia:0.25), (DEXLANSOPRAZOLE:Hypoparathyroidism:0.01), (DIVALPROEX SODIUM:Hypoparathyroidism:0.29), (EVOLOCUMAB:Hypoparathyroidism:0.15), (INSULIN HUMAN:Hypoparathyroidism:0.21), (LOVASTATIN:Hypoparathyroidism:0.1), (RABEPRAZO LE SODIUM:Hypoparathyroidism:0.02), (ALCOHOL:Hypophosphataemia:0.01), (ALENDRONIC ACID:Hypophosphataemia:0.12), (ALFACALCIDOL:Hypophosphataemia:0.04), (ANASTROZ OLE:Hypophosphataemia:0.83), (BUPROPION HYDROCHLORIDE:Hypophosphataemia:0.39), (CANDESARTAN:Hypophosphataemia:0.55), (CANDESARTAN CILEXETILHYDROCHLOROTHIAZIDE:Hypophosphataemia:0.37), (CERTOLIZUMAB PEGOL:Hypophosphataemia:0.12), (COLCHICINE:Hypophosphataemia:0.37), (DASATINIB:H ypophosphataemia:0.88), (DEXLANSOPRAZOLE:Hypophosphataemia:0.71), (DICLOFENAC SODIUM:Hypophosphataemia:0), (DILTIAZEM:Hypophosphataemia:0.55), (DOCETAXEL:Hy pophosphataemia:0.07), (DUTASTERIDE:Hypophosphataemia:0.14), (ELOTUZUMAB:Hypoph osphataemia:0.03), (ESZOPICLONE:Hypophosphataemia:0.39), (FERROUS SULFATEFERROUS SULFATE, DRIED:Hypophosphataemia:1.13), (FLECAINIDE:Hypophosphataemia:0.05), (GLIPIZIDE:Hyp ophosphataemia:0.35), (HYDROCODONE:Hypophosphataemia:0.28), (IMATINIB MESYLATE:Hypophosphataemia:0.27), (INDAPAMIDEPERINDOPRIL:Hypophosphataemia: 0.1), (ISOSORBIDE MONONITRATE:Hypophosphataemia:0.15), (ITRACONAZOLE:Hypophosphataemia:0.16), (LI NAGLIPTIN:Hypophosphataemia:0.12), (LOSARTAN:Hypophosphataemia:0.09), (MELOXICA M:Hypophosphataemia:0.37), (NITROFURANTOIN:Hypophosphataemia:0.29), (OXCARBAZE PINE:Hypophosphataemia:0.02), (PANTOPRAZOLE MAGNESIUM:Hypophosphataemia:0), (PERINDOPRIL:Hypophosphataemia:0.06), (PONATIN 1B:Hypophosphataemia:0.58), (SACUBITRILVALSARTAN:Hypophosphataemia:0.22), (SULF AMETHOXAZOLE:Hypophosphataemia:0.19), (THALIDOMIDE:Hypophosphataemia:0.89), (T IMOLOL:Hypophosphataemia:0.03), (TOPIRAMATE:Hypophosphataemia:0.38), (VINCRISTIN E SULFATE:Hypophosphataemia:0.32), (ALBUTEROL SULFATEIPRATROPIUM BROMIDE:Hypopituitarism:0.56), (AMIODARONE HYDROCHLORIDE:Hypopituitarism:0.5), (ATROPINE SULFATEDIPHENOXYLATE HYDROCHLORIDE:Hypopituitarism:0.36), (BENAZEPRILBENAZEPRIL HYDROCHLORIDE:Hypopituitarism:0.06), (BENZONATATE:Hypopituitarism:0.22), (BORTE ZOMIB:Hypopituitarism:0.06), (CALCIUM CARBONATEVITAMIN D:Hypopituitarism:0.44), (CALCIUM CITRATE:Hypopituitarism:0.79), (CANAKINUMAB:Hypopituitarism:0.03), (CANDESARTAN CILEXETILHYDROCHLOROTHIAZIDE:Hypopituitarism:0.43), (CARFILZOMIB:Hypopituit arism:0.1), (CHONDROITIN SULFATE (BOVINE)GLUCOSAMINE HYDROCHLORIDE:Hypopituitarism:0.17), (CIMETIDINE:Hypopituitarism:0.28), (CIPROFLO XACIN HYDROCHLORIDE:Hypopituitarism:0.81), (CLOZAPINE:Hypopituitarism:0.36), (DACLIZU MAB:Hypopituitarism:0.34), (DASATINIB:Hypopituitarism:0.46), (DEVICE:Hypopituitarism:0.43), (DILTIAZEM:Hypopituitarism:0.43), (DIMENHYDRINATE:Hypopituitarism:0.15), (DOCU SATE CALCIUM:Hypopituitarism:0.11), (DOFETILIDE:Hypopituitarism:0.04), (DOXEPIN HYDROCHLORIDE:Hypopituitarism:0.21), (EDOXABAN TOSYLATE:Hypopituitarism:0.08), (ENOXAPARIN:Hypopituitarism:1.01), (ETORICOXIB:Hy popituitarism:0.01), (EVEROLIMUS:Hypopituitarism:0.47), (EVOLOCUMAB:Hypopituitarism: 0.86), (FEBUXOSTAT:Hypopituitarism:0.19), (FESOTERODINE FUMARATE:Hypopituitarism:0.38), (FLUTICASONE FUROATEVILANTEROL TRIFENATATE:Hypopituitarism:0.13), (GLYBURIDE:Hypopituitarism:0.32), (HEPARIN SODIUM:Hypopituitarism:0.09), (HERBALSTURMERIC:Hypopituitarism:0.05), (HYDRALA ZINE HYDROCHLORIDE:Hypopituitarism:0.15), (HYDROCHLOROTHIAZIDEIRBESARTAN:Hy popituitarism:0.55), (HYDROCHLOROTHIAZIDELISINOPRIL:Hypopituitarism:0.76), (IBRU TINIB:Hypopituitarism:0.53), (IMATINIB MESYLATE:Hypopituitarism:0.28), (IMIPRAMINE HYDROCHLORIDE:Hypopituitarism:0.26), (INDAPAMIDE:Hypopituitarism:0.03), (INDOME THACIN:Hypopituitarism:0.18), (ISOSORBIDE:Hypopituitarism:0.36), (LABETALOLLABET ALOL HYDROCHLORIDE:Hypopituitarism:0.19), (LERCANIDIPINE HYDROCHLORIDE:Hypopituitarism:0.03), (LITHIUM:Hypopituitarism:0.39), (LOVASTATIN:Hypopituitarism:1.39), (METHIMAZOLE:Hypopituitarism:0.07), (METHYLPREDNISOLONE ACETATE:Hypopituitarism:0.64), (METRONIDAZOLEMETRONIDAZOLE HYDROCHLORIDE:Hypopituitarism:0.72), (NITROFURANTOIN:Hypopituitarism:0.64), (NO RETHINDRONE:Hypopituitarism:0.26), (NORTRIPTYLINE:Hypopituitarism:0.61), (PANTOP RAZOLE:Hypopituitarism:1.48), (PAROXETINE:Hypopituitarism:0.68), (PAROXETINEPAR OXETINE HYDROCHLORIDE:Hypopituitarism:0.7), (PAZOPANIB HYDROCHLORIDE:Hypopituitarism:0.1), (PEGFILGRASTIM:Hypopituitarism:0.39), (PENTO SAN POLYSULFATE SODIUM:Hypopituitarism:0.2), (PITAVASTATIN CALCIUM:Hypopituitarism:0.05), (PONATINIB:Hypopituitarism:0.01), (PRIMIDONE:Hypopit uitarism:0.85), (RABEPRAZOLE:Hypopituitarism:0.16), (RABEPRAZOLE SODIUM:Hypopituitarism:0.55), (RALOXIFENE HYDROCHLORIDE:Hypopituitarism:0.37), (ROFLUMILAST:Hypopituitarism:0.14), (SECUKI NUMAB:Hypopituitarism:0.2), (SENNA LEAFSENNOSIDESSENNOSIDES A AND B:Hypopituitarism:0.02), (SODIUM OXYBATE:Hypopituitarism:1.51), (SUMATRIPTANSUMATRIPTAN SUCCINATE:Hypopituitarism:1.31), (TEMAZEPAM:Hypopituitarism:0.88), (TETRACYCLINE:Hypopituitarism:0.23), (TOCILIZUMAB:Hypopituitarism:0.22), (TRIAMTERENE:Hypopituitar ism:0.07), (UMECLIDINIUM BROMIDEVILANTEROL TRIFENATATE:Hypopituitarism:0.51), (VARENICLINE TARTRATE:Hypopituitarism:1.17), (VILAZODONE HYDROCHLORIDE:Hypopituitarism:0.2), (ZONISAMIDE:Hypopituitarism:0.15), (ACETAMI NOPHENOXYCODONEOXYCODONE HYDROCHLORIDE:Hypotension:1.05), (ACETAMINOPHENPROPOXYPHENE HYDROCHLORIDE:Hypotension:0.42), (ACETAMINOPHENPROPOXYPHENE NAPSYLATE:Hypotension:0.23), (AGALSIDASE BETA:Hypotension:0.6), (ALBIGLUTIDE:Hypotension:0.16), (ALCOHOL:Hypotension:0.72), (ALFACALCIDOL:Hypotension:1.43), (ALISKIREN:Hypotension:0.09), (ALISKIREN HEMIFUMARATE:Hypotension:0.26), (AMINO ACIDSDEXTROSEELECTROLYTES NOS:Hypotension:0.57), (AMINO ACIDSDEXTROSEELECTROLYTES NOSSOYBEAN OILVITAMINS:Hypotension:0.11), (AMLODIPINE BESYLATEATORVASTATIN CALCIUM:Hypotension:0), (AMLODIPINE BESYLATEHYDROCHLOROTHIAZIDEOLMESARTAN MEDOXOMIL:Hypotension:0.2), (AMLODIPINEVALSARTAN:Hypotension:0.76), (AMMON IUM LACTATE:Hypotension:0.33), (AXITINIB:Hypotension:0.13), (AZELNIDIPINE:Hypotension:0. 02), (BACITRACIN:Hypotension:0.37), (BECLOMETHASONE:Hypotension:0.06), (BELATAC EPT:Hypotension:0.54), (BENAZEPRILBENAZEPRIL HYDROCHLORIDE:Hypotension:1.02), (BENZTROPINE:Hypotension:0.1), (BETAMETHAS ONE DIPROPIONATECALCIPOTRIENECALCIPOTRIENE HYDRATE:Hypotension:0.05), (BILASTINE:Hypotension:0.21), (BLESELUMAB:Hypotension: 0.2), (BUDESONIDEFORMOTEROL FUMARATE:Hypotension:0.68), (BUPIVACAINEEPINEPHRINE:Hypotension:0.12), (BUPRE NORPHINE HYDROCHLORIDENALOXONE HYDROCHLORIDE:Hypotension:0.7), (CALCIUM CARBONATEVITAMIN D:Hypotension:0.51), (CALCIUMMAGNESIUM:Hypotension:0.04), (CANAGLIFLOZINMET FORMIN HYDROCHLORIDE:Hypotension:0.57), (CANDESARTAN CILEXETILHYDROCHLOROTHIAZIDE:Hypotension:1.64), (CAPSAICIN:Hypotension:0.07), (CARBIMAZOLE:Hypotension:0.19), (CASANTHRANOLDOCUSATE SODIUM:Hypotension:0.19), (CEFOTAXIMECEFOTAXIME SODIUM:Hypotension:0.36), (CETIRIZINE HYDROCHLORIDEPSEUDOEPHEDRINE HYDROCHLORIDE:Hypotension:0.15), (CETUXIMAB:Hypotension:0.76), (CHLORAMBUCI L:Hypotension:0.7), (CICLESONIDE:Hypotension:0.29), (CLINDAMYCIN HYDROCHLORIDE:Hypotension:0.1), (CLINDAMYCIN PHOSPHATE:Hypotension:0.35), (CLOBAZAM:Hypotension:0.68), (CLOMIPRAMINE HYDROCHLORIDE:Hypotension:0.2), (CODEINE PHOSPHATEGUAIFENESIN:Hypotension:0.13), (COLISTIMETHATE SODIUM:Hypotension:0.24), (COLISTIN:Hypotension:0.23), (CYPROHEPTADINE:Hypotensi on:0.65), (DACLIZUMAB:Hypotension:1.6), (DALTEPARIN SODIUM:Hypotension:1.14), (DANAZOL:Hypotension:0.57), (DANTROLENE SODIUM:Hypotension:0.23), (DAPAGLIFLOZIN PROPANEDIOL:Hypotension:0.13), (DAPSONE:Hypotension:1.2), (DEFLAZACORT:Hypoten sion:0.82), (DESVENLAFAXINE SUCCINATE:Hypotension:0.84), (DEXAMETHASONE SODIUM PHOSPHATE:Hypotension:0.42), (DIGITOXIN:Hypotension:0.45), (DOLASETRON MESYLATE:Hypotension:0.18), (ECONAZOLE:Hypotension:0.52), (EDOXABAN TOSYLATE:Hypotension:1.22), (ELTROMBOPAG OLAMINE:Hypotension:0.14), (EMPAGLIFLOZINLINAGLIPTIN:Hypotension:0.32), (EPIRU BICIN:Hypotension:0.81), (EVOLOCUMAB:Hypotension:1.12), (EXEMESTANE:Hypotension: 0.34), (FENOTEROL HYDROBROMIDEIPRATROPIUM BROMIDE:Hypotension:0.05), (FERROUS SULFATEGLYCINE:Hypotension:0.41), (FESOTERODINE FUMARATE:Hypotension:1.07), (FEXOFENADINEPSEUDOEPHEDRINE:Hypotension:0.05), (FIBRINOGEN HUMAN:Hypotension:0.64), (FLURAZEPAMFLURAZEPAM HYDROCHLORIDE:Hypotension:0.26), (FLUTICASONE FUROATEVILANTEROL TRIFENATATE:Hypotension:1.31), (FLUVOXAMINE MALEATE:Hypotension:0.51), (FONDAPARINUX:Hypotension:0.15), (FORMOTEROL FUMARATE:Hypotension:0.77), (FORMOTEROL FUMARATEMOMETASONE FUROATE:Hypotension:0.02), (GIMERACILOTERACILTEGAFUR:Hypotension:0.36), (GL YCOPYRROLATEINDACATEROL MALEATE:Hypotension:0.02), (GLYCOPYRRONIUMINDACATEROL:Hypotension:0.91), (GUANFACINE:Hypotension:0.2), (GUSPERIMUS TRIHYDROCHLORIDE:Hypotension:0.38), (HERBALSTURMERIC:Hypotension:0.27), (HY DROCHLOROTHIAZIDEIRBESARTAN:Hypotension:1.43), (HYDROCODONE BITARTRATEIBUPROFEN:Hypotension:0.01), (HYDROXYCHLOROQUINE DIPHOSPHATE:Hypotension:0.19), (HYDROXYCHLOROQUINE SULFATE:Hypotension:0.87), (IBRITUMOMAB TIUXETAN:Hypotension:0.13), (ILOPROST:Hypotension:1.26), (IMATINIB MESYLATE:Hypotension:1.41), (INDAPAMIDEPERINDOPRIL:Hypotension:1.12), (INOTUZ UMAB OZOGAMICIN:Hypotension:0.14), (INTERFERON GAMMA-1B:Hypotension:0.19), (IRINOTECAN HYDROCHLORIDE:Hypotension:0.67), (LACTASE:Hypotension:0.23), (LERCANIDIPINE:Hy potension:2.12), (LEVALBUTEROL:Hypotension:0.07), (LEVOLEUCOVORIN CALCIUM:Hypotension:0.57), (LOMITAPIDE MESYLATE:Hypotension:0.26), (LORMETAZEPAM:Hypotension:0.05), (LURASIDONE HYDROCHLORIDE:Hypotension:0.03), (MEPERIDINE:Hypotension:0.79), (METFORMIN HYDROCHLORIDEVILDAGLIPTIN:Hypotension:0.27), (METHENAMINE HIPPURATE:Hypotension:1.27), (METILDIGOXIN:Hypotension:0.45), (MIGLUSTAT:Hypote nsion:0.05), (MIZORIBINE:Hypotension:0.17), (MORPHINEMORPHINE HYDROCHLORIDEMORPHINE SULFATEMORPHINE TARTRATE:Hypotension:0.06), (NATEGLINIDE:Hypotension:0.35), (NORTRIPTYLINE HYDROCHLORIDE:Hypotension:0.36), (OLAPARIB:Hypotension:0.05), (OLODATEROL HYDROCHLORIDETIOTROPIUM BROMIDE MONOHYDRATE:Hypotension:0.54), (PANCURONIUM:Hypotension:0.19), (PANTOPRAZO LE MAGNESIUM:Hypotension:1.24), (PARICALCITOL:Hypotension:0.7), (PASIREOTIDE:Hypot ension:0.5), (PAZOPANIB HYDROCHLORIDE:Hypotension:1.17), (PEGINTERFERON ALFA-2B:Hypotension:0.31), (PEGINTERFERON BETA-1A:Hypotension:0.67), (PEMETREXED DISODIUM:Hypotension:0.78), (PENICILLIN V:Hypotension:0.67), (PENTAMIDINE ISETHIONATE:Hypotension:0.35), (PENTOSTATIN:Hypotension:0.26), (PHOSPHOCREATIN E SODIUM:Hypotension:0.03), (PIMOBENDAN:Hypotension:0.25), (PIROXICAM:Hypotension: 0.43), (PITAVASTATIN CALCIUM:Hypotension:1.47), (PLANTAGO SEED:Hypotension:0.62), (POLYETHYLENE GLYCOL 4000:Hypotension:0.22), (PREDNISOLONE SODIUM PHOSPHATE:Hypotension:1.3), (PSYLLIUM HUSK:Hypotension:0.24), (QUINAPRIL:Hypotension:0.06), (RADIATION THERAPY:Hypotension:0.33), (RALTEGRAVIR POTASSIUM:Hypotension:0.2), (RAMUCIRUMAB:Hypotension:0.1), (REGORAFENIB:Hypot ension:0.56), (RETINOL:Hypotension:0.61), (ROFLUMILAST:Hypotension:1.03), (SALMETER OL XINAFOATE:Hypotension:0.12), (SARPOGRELATE:Hypotension:0.11), (SELEXIPAG:Hypote nsion:0.73), (SODIUM PHOSPHATE, DIBASICSODIUM PHOSPHATE, MONOBASIC:Hypotension:0.9), (SOTALOL HYDROCHLORIDE:Hypotension:0.1), (SUGAMMADEX SODIUM:Hypotension:0.55), (SUMATRIPTANSUMATRIPTAN SUCCINATE:Hypotension:1.74), (TEDUGLUTIDEWATER:Hypotension:0.03), (TERAZOSIN TERAZOSIN HYDROCHLORIDE:Hypotension:0.08), (TERBINAFINE HYDROCHLORIDE:Hypotension:0.47), (TERBUTALINE SULFATE:Hypotension:1.07), (THEOPHYLLINE ANHYDROUS:Hypotension:0.42), (THIAMYLAL SODIUM:Hypotension:0.84), (TOCOPHEROL:Hypotension:0.18), (TRANDOLAPRIL:Hypoten sion:0.15), (TRIAMTERENE:Hypotension:0.41), (TRICHLORMETHIAZIDE:Hypotension:0.39), (TRIHEXYPHENIDYL:Hypotension:0.06), (TRIMIPRAMINE MALEATE:Hypotension:0.17), (UMECLIDINIUM BROMIDE:Hypotension:0.24), (VILAZODONE HYDROCHLORIDE:Hypotension:0.47), (ZICONOTIDE ACETATE:Hypotension:0.14), (ZINC:Hypotension:0.59), (ZIPRASIDONE HYDROCHLORIDEZIPRASIDONE MESYLATE:Hypotension:0.05), (FISH OIL:Hypothalamo-pituitary disorder:0.35), (.BETA.-HYDROXYISOVALERIC ACIDARGININEGLUTAMINE:Hypothyroidism:0.36), (ABACAVIRLAMIVUDINE:Hypoth yroidism:0.05), (ACAMPROSATE:Hypothyroidism:0.63), (ACETAMINOPHENAPRONALID ECAFFEINEPROPYPHENAZONE:Hypothyroidism:0.16), (ACETAMINOPHENCAFFEINE PROMETHAZINESALICYLAMIDE:Hypothyroidism:0.24), (ACETAMINOPHENCHLORPH ENIRAMINE MALEATEPSEUDOEPHEDRINE HYDROCHLORIDE:Hypothyroidism:1.04), (ACETAMINOPHENDICHLORALPHENAZON EISOMETHEPTENE HYDROCHLORIDE:Hypothyroidism:0.09), (ACETAMINOPHENPHENYLEPHRINE HYDROCHLORIDE:Hypothyroidism:0.08), (ACETYLCYSTEINEBENZALKONIUM CHLORIDETUAMINOHEPTANE SULFATE:Hypothyroidism:0.43), (ACRIVASTINEPSEUDOEPHEDRINE HYDROCHLORIDE:Hypothyroidism:1.05), (ADENINE:Hypothyroidism:0.44), (ALBUTEROL GUAIFENESIN:Hypothyroidism:0.48), (ALBUTEROLIPRATROPIUM BROMIDE:Hypothyroidism:0.5), (ALCLOMETASONE:Hypothyroidism:0.25), (ALDESLEUKI N:Hypothyroidism:0.96), (ALFALFA:Hypothyroidism:0.53), (AMANTADINE SULFATE:Hypothyroidism:0.1), (AMDINOCILLIN PIVOXIL HYDROCHLORIDE:Hypothyroidism:0.36), (AMINO ACIDSDEXTROSEELECTROLYTES NOS:Hypothyroidism:2.87), (AMITRIPTYLINEPERPHENAZINE:Hypothyroidism:0.05), (AMOXAPINE: Hypothyroidism:0.87), (AMOXICILLIN SODIUM:Hypothyroidism:0.22), (AMOXICILLINCLARITHROMYCINESOMEPRAZOLE:H ypothyroidism:2.24), (AMPHETAMINEDEXTROAMPHETAMINE:Hypothyroidism:0.06), (ASCORBIC ACID/CHROMIUM/SELENIUM/VANADYL SULFATE:Hypothyroidism:1.31), (ASCORBIC ACIDBIOTINCALCIUM PANTOTHENATECYANOCOBALAMINFOLIC ACIDNIACINAMIDEPYRIDOXINE HYDROCHLORIDERIBOFLAVINTHIAMINE HYDROCHLORIDE:Hypothyroidism:0.38), (ASCORBIC ACIDIRON:Hypothyroidism:0.23), (ASENAPINE:Hypothyroidism:0.24), (ASPARAGINASE:H ypothyroidism:0.63), (ASPIRINCAFFEINESALICYLAMIDE:Hypothyroidism:0.54), (ASPIRI NCARISOPRODOL:Hypothyroidism:0.1), (ASPIRINCLOPIDOGREL BISULFATE:Hypothyroidism:0.32), (ATAZANAVIRRITONAVIR:Hypothyroidism:0.1), (ATR ACURIUM:Hypothyroidism:0.35), (ATRACURIUM BESYLATE:Hypothyroidism:0.17), (ATROPINE SULFATE:Hypothyroidism:1.66), (BACITRACINNEOMYCINPOLYMYXIN B:Hypothyroidism:0.32), (BACLOFENCYCLOBENZAPRINEDICLOFENACGABAPENTIN TETRACAINE:Hypothyroidism:0.33), (BELATACEPT:Hypothyroidism:1.49), (BENFOTIAMI NE:Hypothyroidism:0.2), (BENIDIPINE:Hypothyroidism:0.78), (BENZBROMARONE:Hypothy roidism:0.88), (BENZOIC ACIDHYOSCYAMINE SULFATEMETHENAMINEMETHYLENE BLUEPHENYL SALICYLATE:Hypothyroidism:0.13), (BEPOTASTINEBEPOTASTINE BESILATE:Hypothyroidism:0.06), (BEPRIDIL:Hypothyroidism:1.9), (BEPRIDIL HYDROCHLORIDE:Hypothyroidism:0.1), (BETAMETHASONE ACETATEBETAMETHASONE SODIUM PHOSPHATE:Hypothyroidism:0.5), (BETAMETHASONE DIPROPIONATEBETAMETHASONE SODIUM PHOSPHATE:Hypothyroidism:0.5), (BETAMETHASONE DIPROPIONATECALCIPOTRIENE HYDRATE:Hypothyroidism:1.37), (BETAMETHASONE DIPROPIONATEGENTAMICIN SULFATE:Hypothyroidism:0.19), (BETAMETHASONECALCIPOTRIENE:Hypothyroidism:2. 41), (BIFIDOBACTERIUM LONGUM:Hypothyroidism:0.09), (BISMUTH SUBSALICYLATEGLYCERINPROPYLENE GLYCOL:Hypothyroidism:0.31), (BIXALOMER:Hypothyroidism:0.14), (BORIC ACID:Hypothyroidism:0.42), (BROMHEXINE:Hypothyroidism:0.92), (BUTORPHANOL:Hypot hyroidism:0.8), (CAFFEINEISOMETHEPTENEMETHIMAZOLE:Hypothyroidism:0.63), (CAJ UPUT OILEUCALYPTUS OILPEPPERMINT OIL:Hypothyroidism:0.64), (CALCIUM ACETATEMAGNESIUM ACETATEPOTASSIUM ACETATESODIUM ACETATESODIUM CHLORIDE:Hypothyroidism:0.37), (CALCIUM CARBONATEETIDRONATE DISODIUM:Hypothyroidism:1.14), (CALCIUM GLYCEROPHOSPHATE:Hypothyroidism:0.53), (CALCIUM PANTOTHENATE:Hypothyroidism:0.24), (CALCIUM PANTOTHENATECYANOCOBALAMINNIACINAMIDEPYRIDOXINE HYDROCHLORIDERIBOFLAVINTHIAMINE MONONITRATE:Hypothyroidism:1.39), (CALCIUM PHOSPHATE:Hypothyroidism:0.33), (CALCIUMERGOCALCIFEROL:Hypothyroidism:0.03), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:Hypothyroidism:0.19), (CAMPHORMENTHOLMETHYL SALICYLATE:Hypothyroidism:0.12), (CANNABIS SATIVA SUBSP. SATIVA FLOWERING TOP:Hypothyroidism:0.16), (CAPSICUM:Hypothyroidism:0.15), (CARBASPIRIN:Hypothyroidi sm:0.31), (CARMUSTINE:Hypothyroidism:0.68), (CARTEOLOL:Hypothyroidism:0.7), (CEFME TAZOLE SODIUM:Hypothyroidism:0.06), (CEFOTAXIMECEFOTAXIME SODIUM:Hypothyroidism:1.66), (CEFTAZIDIME SODIUM:Hypothyroidism:0.68), (CERIVASTATIN SODIUM:Hypothyroidism:1.79), (CETRORELIX ACETATE:Hypothyroidism:0.25), (CHIMAPHILA UMBELLATAMANGANESE CHLORIDEPOPULUS TREMULOIDESSODIUM TAUROCHOLATE:Hypothyroidism:0.02), (CHLORPROMAZINE HYDROCHLORIDE:Hypothyroidism:0.44), (CHOLINETRAZODONE HYDROCHLORIDE:Hypothyroidism:0.23), (CIFENLINE SUCCINATE:Hypothyroidism:0.57), (CITICOLINE:Hypothyroidism:0.75), (CLADRIBINE:Hyp othyroidism:0.33), (CLENBUTEROL HYDROCHLORIDE:Hypothyroidism:0.84), (CLOCORTOLONE PIVALATE:Hypothyroidism:0.32), (CLOFARABINE:Hypothyroidism:0.14), (CLONIXINCYC LOBENZAPRINE:Hypothyroidism:0.18), (CLOSTRIDIUM TETANICLOSTRIDIUM TETANI TOXOID ANTIGEN (FORMALDEHYDE INACTIVATED)CORYNEBACTERIUM DIPHTHERIAECORYNEBACTERIUM DIPHTHERIAE TOXOID ANTIGEN (FORMALDEHYDE INACTIVATED):Hypothyroidism:0.08), (CLOTIAZEPAM:Hypothyroidism:0.22), (COBALAM IN:Hypothyroidism:0.44), (COBICISTATELVITEGRAVIREMTRICITABINETENOFOVIR: Hypothyroidism:0.53), (COBICISTATELVITEGRAVIREMTRICITABINETENOFOVIR ALAFENAMIDE FUMARATE:Hypothyroidism:1.24), (CODEINE PHOSPHATEDICLOFENAC SODIUM:Hypothyroidism:1.07), (CODEINE PHOSPHATEGUAIFENESINPSEUDOEPHEDRINE HYDROCHLORIDE:Hypothyroidism:0.28), (CODEINE PHOSPHATEPROMETHAZINE:Hypothyroidism:0.51), (CODEINE SULFATE:Hypothyroidism:0.72), (COLESTILAN CHLORIDE:Hypothyroidism:0.14), (COLISTIMETHATE SODIUM:Hypothyroidism:1.14), (COSYNTROPIN:Hypothyroidism:0.6), (COUMARIN:Hypoth yroidism:0.24), (CREATININE:Hypothyroidism:0.11), (CYANOCOBALAMINDICLOFENAC SODIUMPYRIDOXINE HYDROCHLORIDETHIAMINE MONONITRATE:Hypothyroidism:1.09), (CYANOCOBALAMINFOLIC ACIDIRON:Hypothyroidism:0.19), (CYANOCOBALAMINPYRIDOXINE HYDROCHLORIDERIBOFLAVINSODIUM PANTOTHENATETHIAMINE HYDROCHLORIDEVITAMIN B COMPLEX:Hypothyroidism:0.14), (CYPROTERONE:Hypothyroidism:1.01), (CYSTEAMINE BITARTRATE:Hypothyroidism:0.23), (CYSTEINE:Hypothyroidism:0.25), (DARIFENACIN:Hy pothyroidism:0.52), (DARUNAVIRRITONAVIR:Hypothyroidism:0.14), (DEFERIPRONE:Hyp othyroidism:0.99), (DEMECLOCYCLINE:Hypothyroidism:0.14), (DESFLURANE:Hypothyroidi sm:0.85), (DESLORATADINEPSEUDOEPHEDRINE SULFATE:Hypothyroidism:0.07), (DEXAMETHASONENEOMYCINPOLYMYXIN B SULFATE:Hypothyroidism:0.33), (DEXTRAN:Hypothyroidism:0.29), (DEXTRAN 70\HYPROMELLOSE 2910 (4000 MPA.S):Hypothyroidism:0.35), (DEXTROMETHORPHANDEXTROMETHORPHAN HYDROBROMIDEGUAIFENESIN:Hypothyroidism:0.26), (DICHLORPHENAMIDE:Hypothy roidism:0.32), (DIENOGEST:Hypothyroidism:2.18), (DIHYDROCODEINE PHOSPHATE:Hypothyroidism:0.71), (DILAZEP:Hypothyroidism:1.04), (DIMEMORFAN:Hypo thyroidism:0.3), (DIMETHYL SULFOXIDE:Hypothyroidism:0.98), (DIPHENHYDRAMINE HYDROCHLORIDEDOXYLAMINE SUCCINATE:Hypothyroidism:0.59), (DIPHENHYDRAMINE HYDROCHLORIDEPHENYLEPHRINE HYDROCHLORIDE:Hypothyroidism:0.33), (DIPHTHERIA AND TETANUS TOXOIDS AND ACELLULAR PERTUSSIS VACCINE ADSORBED:Hypothyroidism:0.85), (DOLASETRON MESYLATE:Hypothyroidism:1.95), (DOXERCALCIFEROL:Hypothyroidism:0.5), (DOXYCYC LINE HYDROCHLORIDE:Hypothyroidism:0.97), (DOXYLAMINE SUCCINATEPYRIDOXINE HYDROCHLORIDE:Hypothyroidism:1.11), (ECABET:Hypothyroidism:0.29), (ECABET SODIUM:Hypothyroidism:0.38), (ELCATONIN:Hypothyroidism:0.07), (ELOSULFASE ALFA:Hypothyroidism:0.09), (EPALRESTAT:Hypothyroidism:0.72), (EPHEDRINE HYDROCHLORIDE:Hypothyroidism:0.29), (EPHEDRINE HYDROCHLORIDEGUAIFENESIN:Hypothyroidism:0.49), (ERIBULIN:Hypothyroidism:1.2), (ERYTHROMYCIN LACTOBIONATE:Hypothyroidism:0.04), (ESCHERICHIA COLI:Hypothyroidism:0.37), (ESMOLOL HYDROCHLORIDE:Hypothyroidism:1.91), (ESTRADIOL ACETATE:Hypothyroidism:0.95), (ESTRADIOL BENZOATE:Hypothyroidism:0.84), (ETHINYL ESTRADIOL:Hypothyroidism:0.27), (ETHINYL ESTRADIOLETHYNODIOL:Hypothyroidism:0.11), (ETHINYL ESTRADIOLFERROUS FUMARATENORETHINDRONE ACETATE:Hypothyroidism:0.94), (ETHINYL ESTRADIOLLEVONORGESTREL ACETATE:Hypothyroidism:1.29), (ETHIODIZED OIL:Hypothyroidism:0.36), (ETHOSUXIMIDE:Hypothyroidism:0.58), (ETOPOSIDE PHOSPHATE:Hypothyroidism:0.94), (ETRETINATE:Hypothyroidism:2.34), (FAROPENEM SODIUM:Hypothyroidism:0.46), (FELODIPINEMETOPROLOL SUCCINATE:Hypothyroidism:0.48), (FENOPROFEN CALCIUM:Hypothyroidism:0.43), (FERRIC HYDROXIDE:Hypothyroidism:1.44), (FERRIC POLYSACCHARIDE COMPLEX:Hypothyroidism:0.68), (FERROUS SULFATEGLYCINE:Hypothyroidism:2.81), (FILGRASTIM-SNDZ:Hypothyroidism:0.41), (FLOMOXEF:Hypothyroidism:0.08), (FLUCYTOSINE:Hypothyr oidism:0.11), (FLUOROURACILIRINOTECANLEUCOVORIN:Hypothyroidism:0.08), (FLUO ROURACILLEUCOVORIN CALCIUMOXALIPLATIN:Hypothyroidism:0.79), (FLUPENTIXOL DECANOATE:Hypothyroidism:0.07), (FOLIC ACIDIRON POLYMALTOSE:Hypothyroidism:0.02), (FOSPHENYTOIN SODIUM:Hypothyroidism:0.17), (FOSPHENYTOINFOSPHENYTOIN SODIUM:Hypothyroidism:1.2), (FURSULTIAMINE HYDROCHLORIDE:Hypothyroidism:0.42), (FUSIDATE SODIUM:Hypothyroidism:0.28), (GADOPENTETATE DIMEGLUMINE:Hypothyroidism:0.38), (GADOTERATE MEGLUMINE:Hypothyroidism:0.98), (GADOXETATE DISODIUM:Hypothyroidism:0.35), (GENTIAN VIOLET:Hypothyroidism:0.39), (GLATIRAMER:Hypothyroidism:0.38), (GLIMEPIRIDEROSI GLITAZONE MALEATE:Hypothyroidism:0.7), (GLIPIZIDEMETFORMIN HYDROCHLORIDE:Hypothyroidism:1.55), (GLUCAGON HYDROCHLORIDE:Hypothyroidism:0.23), (GLUCONATE:Hypothyroidism:0.26), (GLYCOP YRROLATEINDACATEROL MALEATE:Hypothyroidism:1.99), (GOLD:Hypothyroidism:0.12), (GUAIFENESINPHENYLE PHRINE HYDROCHLORIDE:Hypothyroidism:0.72), (GUANABENZ ACETATE:Hypothyroidism:0.43), (HALCINONIDE:Hypothyroidism:0.62), (HERBALSHOME OPATHICSGOLDENSEAL:Hypothyroidism:0.09), (HIGH DENSITY POLYETHYLENE:Hypothyroidism:0.87), (HUMAN COLOSTRUM:Hypothyroidism:0), (HYDRALAZINE HYDROCHLORIDEISOSORBIDE DINITRATE:Hypothyroidism:0.52), (HYDRAZINE SULFATE:Hypothyroidism:0.66), (HYDROCHLORIC ACID:Hypothyroidism:0.23), (HYDROCHLOROTHIAZIDEMETOPROLOL TARTRATE:Hypothyroidism:0.84), (HYDROCORTISONE HEMISUCCINATE:Hypothyroidism:0.19), (HYDROCORTISONE VALERATE:Hypothyroidism:0.45), (HYDROQUINONE:Hypothyroidism:0.43), (HYDROXOC OBALAMIN ACETATE:Hypothyroidism:0.09), (HYPERICUM PERFORATUM:Hypothyroidism:0.19), (IBRITUMOMAB TIUXETAN:Hypothyroidism:1.4), (IFENPRODIL:Hypothyroidism:0.48), (IMIDAPRIL:Hypothy roidism:1.48), (INTERFERON ALFA-2A:Hypothyroidism:2.4), (INTERFERON ALFACON-1:Hypothyroidism:0.24), (INTERFERON GAMMA-1B:Hypothyroidism:1.72), (IPRAGLIFLOZIN:Hypothyroidism:0.26), (ISAVUCONAZOLE:Hyp othyroidism:0.01), (ISONIAZIDPYRAZINAMIDERIFAMPIN:Hypothyroidism:1.38), (IVACA FTOR:Hypothyroidism:1.1), (KALLIDINOGENASE:Hypothyroidism:0.38), (LACTOPEROXID ASE BOVINELYSOZYME HYDROCHLORIDE:Hypothyroidism:0.15), (LACTOSE:Hypothyroidism:0.21), (LANOLINMI NERAL OILPETROLATUM:Hypothyroidism:0.09), (LARONIDASE:Hypothyroidism:0.69), (LAVEND ER OIL:Hypothyroidism:0.01), (LEUPROLIDE:Hypothyroidism:0.47), (LEVOMEFOLIC ACIDMETHYLCOBALAMINTURMERIC:Hypothyroidism:0.16), (LEVOMILNACIPRAN:H ypothyroidism:0.01), (LINSEED OIL:Hypothyroidism:0.38), (LINSIDOMINE:Hypothyroidism:1.14), (LOMUSTINE:Hypothyroid ism:0.62), (LUMACAFTOR:Hypothyroidism:0.15), (LUTETIUM LU-177: Hypothyroidism:1.6), (LYSOZYME HYDROCHLORIDE:Hypothyroidism:0.57), (MAGNESIUM SULFATEPOTASSIUM SULFATESODIUM SULFATE:Hypothyroidism:0.49), (MALTOSE:Hypothyroidism:0.28), (MANGANESEMANG ANESE CHLORIDE:Hypothyroidism:0.21), (MECLOZINE DIHYDROCHLORIDE:Hypothyroidism:0.59), (METHENAMINE MANDELATE:Hypothyroidism:1.42), (METHSUXIMIDE:Hypothyroidism:0.06), (METHYLE NE BLUE:Hypothyroidism:0.43), (MIGLUSTAT:Hypothyroidism:1.57), (MITIGLINIDE CALCIUM HYDRATEVOGLIBOSE:Hypothyroidism:0.61), (MITOXANTRONE HYDROCHLORIDE:Hypothyroidism:0.8), (MOSAPRIDE CITRATE DIHYDRATE:Hypothyroidism:0.1), (NAFAMOSTAT MESYLATE:Hypothyroidism:0.07), (NAFARELIN ACETATE:Hypothyroidism:0.21), (NALOXONE:Hypothyroidism:1.71), (NELARABINE:Hypot hyroidism:0.24), (NEOSTIGMINE METHYLSULFATE:Hypothyroidism:0.12), (NIAOULI OILQUININE BENZOATETHIAMINE HYDROCHLORIDE:Hypothyroidism:0.09), (NUSINERSEN:Hypothyroidism:0.55), (NYSTATI NTRIAMCINOLONE ACETONIDE:Hypothyroidism:0.26), (OBETICHOLIC ACID:Hypothyroidism:0.53), (OLODATEROL:Hypothyroidism:0.11), (OPIUM:Hypothyroidism:0.04), (OTILONIUM BROMIDE:Hypothyroidism:0.42), (OXACILLIN SODIUM:Hypothyroidism:0.05), (OXANDROLONE:Hypothyroidism:0.71), (PARSLEY:Hypoth yroidism:0.2), (PEGINTERFERON ALFA-2A OR PEGINTERFERON ALFA-2B OR PEGINTERFERON BETA-1A OR PEGINTERFERON LAMBDA-1A:Hypothyroidism:0.34), (PENTAERYTHRITOL TETRANITRATE:Hypothyroidism:0.82), (PENTOSTATIN:Hypothyroidism:1.74), (PEPSIN:Hy pothyroidism:0.41), (PEROSPIRONE:Hypothyroidism:0.23), (PEROSPIRONE HYDROCHLORIDE:Hypothyroidism:0.18), (PHENOTHIAZINE:Hypothyroidism:0.14), (PHEN YLEPHRINE HYDROCHLORIDEPROMETHAZINE HYDROCHLORIDE:Hypothyroidism:0.49), (PHENYLEPHRINE HYDROCHLORIDEWITCH HAZEL:Hypothyroidism:0.82), (PODOFILOX:Hypothyroidism:0.21), (POTASSIUM CHLORIDESODIUM CHLORIDE:Hypothyroidism:0.26), (POTASSIUM CHLORIDESODIUM CHLORIDESODIUM LACTATE:Hypothyroidism:0.06), (POTASSIUM PHOSPHATE, MONOBASIC:Hypothyroidism:1.07), (PRAMOXINE HYDROCHLORIDE:Hypothyroidism:1.08), (PREDNISOLONE SODIUM METAZOATE:Hypothyroidism:0.41), (PRISTINAMYCIN:Hypothyroidism:1.97), (PROBUCOL:Hypothyroidism:1.2), (PROCAINE:Hypothyroidism:0.48), (PROCARBAZINE HYDROCHLORIDE:Hypothyroidism:0.03), (PROMAZINE:Hypothyroidism:0.25), (PROMAZI NE HYDROCHLORIDE:Hypothyroidism:0.56), (PROPANTHELINE BROMIDE:Hypothyroidism:0.02), (PROPIOMAZINE:Hypothyroidism:0.26), (PRUCALOPRID E:Hypothyroidism:0.03), (PSORALEN:Hypothyroidism:0.41), (QUINIDINE GLUCONATE:Hypothyroidism:0.28), (RED CLOVER:Hypothyroidism:0.16), (RED YEAST:Hypothyroidism:0.07), (REGADENOSON:Hypothyroidism:0.23), (RHUBARB:Hypothy roidism:0.72), (RILPIVIRINE:Hypothyroidism:0.44), (RILPIVIRINE HYDROCHLORIDE:Hypothyroidism:1.68), (ROXITHROMYCIN:Hypothyroidism:1.03), (RUFI NAMIDE:Hypothyroidism:0.71), (SARGRAMOSTIM:Hypothyroidism:0.42), (SARPOGRELAT E HYDROCHLORIDE:Hypothyroidism:0.42), (SCOPOLAMINE HYDROBROMIDE:Hypothyroidism:0.2), (SEA WATER:Hypothyroidism:0.01), (SERTINDOLE:Hypothyroidism:0.22), (SILICON DIOXIDE:Hypothyroidism:1.75), (SITAFLOXACIN:Hypothyroidism:0.08), (SODIUM:Hypothy roidism:0.96), (SODIUM FLUORIDEVITAMINS:Hypothyroidism:0.03), (SODIUM PHOSPHATE, DIBASIC, ANHYDROUSSODIUM PHOSPHATE, DIBASIC, HEPTAHYDRATESODIUM PHOSPHATE, MONOBASIC, MONOHYDRATE:Hypothyroidism:0.46), (SODIUM PHOSPHATE, MONOBASIC, ANHYDROUS:Hypothyroidism:0.69), (STREPTOMYCINSTREPTOMYCIN SULFATE:Hypothyroidism:0.53), (SUGAMMADEX:Hypothyroidism:0.24), (SULBUTIAMINE:Hypothyroidism:0.25), (SUPLATAST TOSILATE:Hypothyroidism:1.62), (TABALUMAB:Hypothyroidism:0.11), (TBO-FILGRASTIM:Hypothyroidism:0.44), (TEGAFURURACIL:Hypothyroidism:0.21), (TENELIG LIPTIN:Hypothyroidism:1.18), (THIAMYLAL SODIUM:Hypothyroidism:0.1), (THIOTHIXENE HYDROCHLORIDE:Hypothyroidism:0.07), (THYMOL:Hypothyroidism:0.01), (TIANEPTINE SODIUM:Hypothyroidism:0.74), (TIAPRIDE:Hypothyroidism:0.03), (TIAPROFENIC ACID:Hypothyroidism:0.42), (TOFISOPAM:Hypothyroidism:0.13), (TRANYLCYPROMINE SULFATE:Hypothyroidism:0.56), (TRICLOSAN:Hypothyroidism:0.02), (TROGLITAZONE:Hy pothyroidism:0.12), (TROXIPIDE:Hypothyroidism:0.39), (URACIL:Hypothyroidism:0.07), (VAL RUBICIN:Hypothyroidism:0.64), (VINBLASTINE:Hypothyroidism:0.35), (VITAMIN A PALMITATE:Hypothyroidism:0.23), (VORAPAXAR SULFATE:Hypothyroidism:0.47), (WHEY:Hypothyroidism:0.04), (YTTRIUM Y-90:Hypothyroidism:0.01), (ZANAMIVIR:Hypothyroidism:0.11), (ZUCLOPENTHIXOL DECANOATE:Hypothyroidism:0.73), (ALBUTEROL SULFATEIPRATROPIUM BROMIDE:Hypotonia:0.25), (ALCOHOL:Hypotonia:0.27),(ARMODAFINIL:Hypotonia:0.53), (BISOPROLOL FUMARATE:Hypotonia:0.46), (BUPIVACAINEEPINEPHRINE:Hypotonia:0.24), (BUPRENO RPHINE HYDROCHLORIDE:Hypotonia:0.21), (COLCHICINE:Hypotonia:0.41), (DACLIZUMAB:Hypo tonia:0), (DIPHENHYDRAMINE:Hypotonia:1.26), (DRONABINOL:Hypotonia:0.01), (ELETRIP TAN HYDROBROMIDE:Hypotonia:0.17), (ERGOCALCIFEROL:Hypotonia:0.46), (EVEROLIMUS: Hypotonia:0.28), (EVOLOCUMAB:Hypotonia:0.51), (FERROUS SULFATE:Hypotonia:0.77), (FERROUS SULFATEFERROUS SULFATE, DRIED:Hypotonia:0.74), (FLUTICASONE:Hypotonia:0.09), (GLIMEPIRIDE:Hypotonia:0.37), (HYDROCHLOROTHIAZIDELISINOPRIL:Hypotonia:0.33), (IBRUTINIB:Hypotonia:0.69), (IP RATROPIUM:Hypotonia:0.09), (LIDOCAINEPRILOCAINE:Hypotonia:0.26), (LISDEXAMFE TAMINE DIMESYLATE:Hypotonia:0.46), (LITHIUM CARBONATE:Hypotonia:0.29), (METHYLPREDNISOLONE ACETATE:Hypotonia:0.55), (METRONIDAZOLEMETRONIDAZOLE HYDROCHLORIDE:Hypotonia:0.54), (NALOXONE:Hypotonia:0.03), (NEBIVOLOL:Hypotoni a:0.21), (NILOTINIB:Hypotonia:0.03), (PAMIDRONATE DISODIUM:Hypotonia:0.06), (PENICILLIN:Hypotonia:0.25), (PRIMIDONE:Hypotonia:0.42), (P ROGESTERONE:Hypotonia:0.38), (RABEPRAZOLE SODIUM:Hypotonia:0.2), (RANOLAZINE:Hypotonia:0.36), (RISEDRONATE SODIUM:Hypotonia:0.02), (ROPINIROLE HYDROCHLORIDE:Hypotonia:0.18), (SACUBITRILVALSARTAN:Hypotonia:0.27), (SECU KINUMAB:Hypotonia:0.66), (SUMATRIPTANSUMATRIPTAN SUCCINATE:Hypotonia:0.13), (VARENICLINE TARTRATE:Hypotonia:1.14), (VERAPAMIL HYDROCHLORIDE:Hypotonia:0.5), (VITAMIN B:Hypotonia:0.41), (.ALPHA.1-PROTEINASE INHIBITOR HUMAN:Hypovitaminosis:0.57), (ABIRATERONE ACETATE:Hypovitaminosis:0.28), (ACARBOSE:Hypovitaminosis:0.65), (ACETAMINOPHEN OXYCODONEOXYCODONE HYDROCHLORIDE:Hypovitaminosis:0.52), (ACETAMINOPHENPROPOXYPHENE HYDROCHLORIDE:Hypovitaminosis:0.31), (ACETAMINOPHENTRAMADOL:Hypovitamin osis:0.08), (ACETAZOLAMIDE:Hypovitaminosis:0.83), (ADO-TRASTUZUMAB EMTANSINE:Hypovitaminosis:0.57), (ALBIGLUTIDE:Hypovitaminosis:0.02), (ALCOHOL:Hy povitaminosis:0.56), (ALENDRONIC ACID:Hypovitaminosis:0.46), (ALFUZOSIN HYDROCHLORIDE:Hypovitaminosis:0.21), (AMANTADINE HYDROCHLORIDE:Hypovitaminosis:0.18), (AMIKACIN:Hypovitaminosis:0.23), (AMISULPR IDE:Hypovitaminosis:0.56), (AMLODIPINE BESYLATEATORVASTATIN CALCIUM:Hypovitaminosis:0.02), (AMLODIPINE BESYLATEHYDROCHLOROTHIAZIDEOLMESARTAN MEDOXOMIL:Hypovitaminosis:0.06), (AMLODIPINEVALSARTAN:Hypovitaminosis:0.08), (AXITINIB:Hypovitaminosis:0.16), (BENAZEPRILBENAZEPRIL HYDROCHLORIDE:Hypovitaminosis:0.51), (BETAMETHASONE DIPROPIONATE:Hypovitaminosis:0.08), (BICALUTAMIDE:Hypovitaminosis:0.68), (BRINZO LAMIDE:Hypovitaminosis:0.31), (BUMETANIDE:Hypovitaminosis:1.62), (CALCITONIN:Hyp ovitaminosis:0.06), (CALCITONIN SALMON:Hypovitaminosis:0.04), (CALCIUMMAGNESIUM:Hypovitaminosis:0.04), (CANAG LIFLOZINMETFORMIN HYDROCHLORIDE:Hypovitaminosis:0.61), (CANAKINUMAB:Hypovitaminosis:0.44), (CAR VEDILOL PHOSPHATE:Hypovitaminosis:1.01), (CEFOTAXIME SODIUM:Hypovitaminosis:0.55), (CICLESONIDE:Hypovitaminosis:0.75), (CLOMIPRAMINE HYDROCHLORIDE:Hypovitaminosis:0.08), (COD LIVER OIL:Hypovitaminosis:0.08), (CORTICOTROPIN:Hypovitaminosis:0.48), (CORTISONEHYDR OCORTISONE:Hypovitaminosis:1.27), (DACLIZUMAB:Hypovitaminosis:1.12), (DANAZOL:H ypovitaminosis:0.68), (DASABUVIROMBITASVIRPARITAPREVIRRITONAVIR:Hypovita minosis:0.27), (DESONIDE:Hypovitaminosis:0.37), (DIMENHYDRINATE:Hypovitaminosis:0.7 3), (DIPYRIDAMOLE:Hypovitaminosis:0.06), (DOFETILIDE:Hypovitaminosis:0.67), (DOXAZ OSINDOXAZOSIN MESYLATE:Hypovitaminosis:1.09), (DOXEPIN:Hypovitaminosis:0.39), (DOXEPIN HYDROCHLORIDE:Hypovitaminosis:0.52), (DRONEDARONE:Hypovitaminosis:0.21), (DUT ASTERIDE:Hypovitaminosis:1.14), (ECONAZOLE:Hypovitaminosis:0.3), (EFAVIRENZEMT RICITABINETENOFOVIR DISOPROXIL FUMARATE:Hypovitaminosis:0.14), (ELETRIPTAN HYDROBROMIDE:Hypovitaminosis:1.22), (ELOTUZUMAB:Hypovitaminosis:0.09), (EMPAG LIFLOZINLINAGLIPTIN:Hypovitaminosis:0.7), (ESTROGENS, CONJUGATEDMEDROXYPROGESTERONE ACETATE:Hypovitaminosis:0.66), (FEXOFENADINEPSEUDOEPHEDRINE:Hypovitaminosi s:0.14), (FORMOTEROL:Hypovitaminosis:0.58), (FORMOTEROL FUMARATE:Hypovitaminosis:0.8), (FORMOTEROL FUMARATEMOMETASONE FUROATE:Hypovitaminosis:0.55), (FUSIDIC ACID:Hypovitaminosis:0.48), (GLYCOPYRROLATE:Hypovitaminosis:0.25), (GLYCOPYRRO NIUMINDACATEROL:Hypovitaminosis:0.79), (GUANFACINE:Hypovitaminosis:0.02), (GUA NFACINE HYDROCHLORIDE:Hypovitaminosis:0.03), (HERBALSTURMERIC:Hypovitaminosis:0.5), (H YDROXYCHLOROQUINE DIPHOSPHATE:Hypovitaminosis:0.32), (ILOPROST:Hypovitaminosis:0.48), (IMATINIB MESYLATE:Hypovitaminosis:1.58), (IMIPRAMINE HYDROCHLORIDE:Hypovitaminosis:0.42), (INDAPAMIDE:Hypovitaminosis:0.99), (INDAPA MIDEPERINDOPRIL:Hypovitaminosis:1.08), (INTERFERON:Hypovitaminosis:0.14), (INTER FERON ALFA-2A:Hypovitaminosis:0.12), (INTERFERON ALFA-2B:Hypovitaminosis:0.17), (IXAZOMIB:Hypovitaminosis:0.28), (LABETALOL HYDROCHLORIDE:Hypovitaminosis:0.02), (LIDOCAINEPRILOCAINE:Hypovitaminosis:0.5 6), (MEPOLIZUMAB:Hypovitaminosis:0.47), (METAXALONE:Hypovitaminosis:1.26), (METF ORMIN HYDROCHLORIDEVILDAGLIPTIN:Hypovitaminosis:0.21), (METHIMAZOLE:Hypovitamin osis:0.19), (METHYLPREDNISOLONE ACETATE:Hypovitaminosis:0.76), (MOXONIDINE:Hypovitaminosis:0.11), (NINTEDANIB:Hy povitaminosis:0.63), (NIRAPARIB:Hypovitaminosis:0.11), (OLODATEROL HYDROCHLORIDETIOTROPIUM BROMIDE MONOHYDRATE:Hypovitaminosis:0.32), (OSELTAMIVIR PHOSPHATE:Hypovitaminosis:0.07), (OXCARBAZEPINE:Hypovitaminosis:2.31), (PAMIDRO NATE DISODIUM:Hypovitaminosis:0.01), (PANTOPRAZOLE MAGNESIUM:Hypovitaminosis:1.56), (PARICALCITOL:Hypovitaminosis:0.13), (PATIROME R:Hypovitaminosis:0.16), (PHENYTOIN SODIUM:Hypovitaminosis:0.06), (PIMECROLIMUS:Hypovitaminosis:0.09), (PITAVASTATIN CALCIUM:Hypovitaminosis:0.64), (PONATINIB:Hypovitaminosis:1.08), (POSACONAZOLE: Hypovitaminosis:0.52), (PRAZOSIN HYDROCHLORIDE:Hypovitaminosis:0.06), (PREDNISOLONE ACETATE:Hypovitaminosis:0.31), (PROPAFENONE HYDROCHLORIDE:Hypovitaminosis:0.06), (QUINAPRIL:Hypovitaminosis:0.12), (RABEPRA ZOLE:Hypovitaminosis:0.73), (REGORAFENIB:Hypovitaminosis:0.2), (RIVASTIGMINE TARTRATE:Hypovitaminosis:0.65), (RIZATRIPTAN BENZOATE:Hypovitaminosis:0.97), (SACUBITRILVALSARTAN:Hypovitaminosis:1.09), (SC OPOLAMINE:Hypovitaminosis:0.84), (SUMATRIPTAN:Hypovitaminosis:0.59), (TERBINAFI NE HYDROCHLORIDE:Hypovitaminosis:0.36), (TETRACYCLINE:Hypovitaminosis:0.89), (TROS PIUM CHLORIDE:Hypovitaminosis:0.15), (UMECLIDINIUM BROMIDE:Hypovitaminosis:0.08), (UMECLIDINIUM BROMIDEVILANTEROL TRIFENATATE:Hypovitaminosis:1.19), (AMIODARONE:Hypoxia:0.47), (AMIODARONE HYDROCHLORIDE:Hypoxia:0.07), (AMITRIPTYLINE:Hypoxia:0.17), (BUMETANIDE:Hypo xia:0.99), (CERTOLIZUMAB PEGOL:Hypoxia:0.33), (CILASTATIN SODIUMIMIPENEM:Hypoxia:0.1), (DASATINIB:Hypoxia:0.03), (DICLOFENAC:Hypoxia:0.1 3), (DICLOFENAC SODIUM:Hypoxia:0.37), (ESZOPICLONE:Hypoxia:0.02), (EVEROLIMUS:Hypoxia:0.16), (HY DROCODONE:Hypoxia:0.23), (ISOSORBIDE MONONITRATE:Hypoxia:0.59), (MELOXICAM:Hypoxia:0.31), (NEBIVOLOL:Hypoxia:0.24), (NILOTINIB:Hypoxia:0.04), (OXCARBAZEPINE:Hypoxia:0.17), (PANTOPRAZOLE MAGNESIUM:Hypoxia:0.04), (POLYETHYLENE GLYCOLS:Hypoxia:0.28), (PRAVASTATIN:Hypoxia:0.36), (PRAVASTATIN SODIUM:Hypoxia:0.3), (PROMETHAZINEPROMETHAZINE HYDROCHLORIDE:Hypoxia:0.05), (RISEDRONATE SODIUM:Hypoxia:0.05), (SACUBITRILVALSARTAN:Hypoxia:0.32), (TEMAZEPAM:Hypox ia:0.07), (TOPIRAMATE:Hypoxia:0.58), (TRIAMCINOLONE ACETONIDE:Hypoxia:0.33), (UMECLIDINIUM BROMIDEVILANTEROL TRIFENATATE:Hypoxia:0.17), (VENLAFAXINE HYDROCHLORIDE:Hypoxia:0.2), (VERAPAMIL HYDROCHLORIDE:Hypoxia:0.3), (AMIODARONE:Hysterectomy:0.42), (ANASTROZOLE:H ysterectomy:0.29), (BUPRENORPHINE HYDROCHLORIDENALOXONE HYDROCHLORIDE:Hysterectomy:0.48), (CANDESARTAN:Hysterectomy:0.04), (CHOLESTY RAMINE:Hysterectomy:0.52), (CIMETIDINE:Hysterectomy:0), (COLCHICINE:Hysterectomy:0 0.33), (DIPHENHYDRAMINE:Hysterectomy:0.53), (DULOXETINE:Hysterectomy:0.46), (ENOX APARIN:Hysterectomy:0.17), (ESZOPICLONE:Hysterectomy:0.36), (FLUTICASONE:Hysterec tomy:0.23), (GLIPIZIDE:Hysterectomy:0.38), (HYDROXYCHLOROQUINE DIPHOSPHATE:Hysterectomy:0.05), (HYOSCYAMINE:Hysterectomy:0.2), (IBRUTINIB:Hyst erectomy:0.66), (IMIPRAMINE HYDROCHLORIDE:Hysterectomy:0.17), (INDOMETHACIN:Hysterectomy:0.64), (LABETAL OLLABETALOL HYDROCHLORIDE:Hysterectomy:0.07), (METHYLPREDNISOLONE ACETATE:Hysterectomy:0.06), (METHYLPREDNISOLONE SODIUM SUCCINATE:Hysterectomy:0.21), (METRONIDAZOLEMETRONIDAZOLE HYDROCHLORIDE:Hysterectomy:0.03), (NIACIN:Hysterectomy:0.05), (NITROFURANTOIN: Hysterectomy:0.09), (NORTRIPTYLINE:Hysterectomy:0.17), (OMEGA-3-ACID ETHYL ESTERS:Hysterectomy:0.18), (OXCARBAZEPINE:Hysterectomy:0.2), (OXYGEN:Hysterectom y:0.47), (PANTOPRAZOLE MAGNESIUM:Hysterectomy:0.06), (RANOLAZINE:Hysterectomy:0.32), (RISEDRONATE SODIUM:Hysterectomy:0.01), (SECUKINUMAB:Hysterectomy:0.49), (TAMSULOSIN:Hystere ctomy:0.82), (TETRACYCLINE:Hysterectomy:0.13), (TRIAMTERENE:Hysterectomy:0.36), (U MECLIDINIUM BROMIDEVILANTEROL TRIFENATATE:Hysterectomy:0.37), (.ALPHA.1-PROTEINASE INHIBITOR HUMAN:Idiopathic pulmonary fibrosis:1.21), (ACARBOSE:Idiopathic pulmonary fibrosis:0.87), (ACETAMINOPHENOXYCODONE HYDROCHLORIDEOXYCODONE TEREPHTHALATE:Idiopathic pulmonary fibrosis:0.84), (ACETAMINOPHENOXYCODONEOXYCODONE HYDROCHLORIDE:Idiopathic pulmonary fibrosis:1.08), (AGALSIDASE BETA:Idiopathic pulmonary fibrosis:0.96), (ALBUTEROLIPRATROPIUM BROMIDE:Idiopathic pulmonary fibrosis:0.05), (ALISKIREN:Idiopathic pulmonary fibrosis:0.78), (AMISULPRIDE:Idiopathic pulmonary fibrosis:0.09), (AMLODIPINE BESYLATEATORVASTATIN CALCIUM:Idiopathic pulmonary fibrosis:0.31), (AMLODIPINE BESYLATEHYDROCHLOROTHIAZIDEVALSARTAN:Idiopathic pulmonary fibrosis:0.34), (ANTIHISTAMINES NOS:Idiopathic pulmonary fibrosis:0.31), (AXITINIB:Idiopathic pulmonary fibrosis:0.23), (AZTREONAM:Idiopathic pulmonary fibrosis:0.2), (BETHANECHOL CHLORIDE:Idiopathic pulmonary fibrosis:0.05), (BILASTINE:Idiopathic pulmonary fibrosis:0.32), (BORTEZOMIB:Idiopathic pulmonary fibrosis:0.54), (BRINZOLAMIDETIMOLOL MALEATE:Idiopathic pulmonary fibrosis:0.1), (BUPRENORPHINE HYDROCHLORIDE:Idiopathic pulmonary fibrosis:0.01), (CALCIPOTRIENE:Idiopathic pulmonary fibrosis:0.09), (CALCITONIN:Idiopathic pulmonary fibrosis:0.4), (CALCIUMMAGNESIUMZINC:Idiopathic pulmonary fibrosis:0.3), (CANAGLIFLOZINMETFORMIN HYDROCHLORIDE:Idiopathic pulmonary fibrosis:0.52), (CANAKINUMAB:Idiopathic pulmonary fibrosis:0.43), (CARBOXYMETHYLCELLULOSE SODIUM:Idiopathic pulmonary fibrosis:0.02), (CARFILZOMIB:Idiopathic pulmonary fibrosis:0.96), (CARTEOLOL HYDROCHLORIDE:Idiopathic pulmonary fibrosis:0.04), (CEFAZOLIN SODIUM:Idiopathic pulmonary fibrosis:0.25), (CEVIMELINE HYDROCHLORIDE:Idiopathic pulmonary fibrosis:0.19), (CHLORDIAZEPOXIDE HYDROCHLORIDECLIDINIUM BROMIDE:Idiopathic pulmonary fibrosis:0.27), (CHLORHEXIDINE:Idiopathic pulmonary fibrosis:0.39), (CHLORPROMAZINE:Idiopathic pulmonary fibrosis:0.01), (DACLIZUMAB:Idiopathic pulmonary fibrosis:0.89), (DANAZOL:Idiopathic pulmonary fibrosis:0.72), (DAPAGLIFLOZIN PROPANEDIOL:Idiopathic pulmonary fibrosis:0.51), (DASATINIB:Idiopathic pulmonary fibrosis:1.59), (DESVENLAFAXINE SUCCINATE:Idiopathic pulmonary fibrosis:0.85), (DEXAMETHASONE ACETATE:Idiopathic pulmonary fibrosis:0.14), (DEXTROAMPHETAMINE SULFATE:Idiopathic pulmonary fibrosis:0.39), (DIPYRONE:Idiopathic pulmonary fibrosis:0.22), (DIVALPROEX SODIUM:Idiopathic pulmonary fibrosis:0.56), (ECONAZOLE:Idiopathic pulmonary fibrosis:0.69), (ELETRIPTAN HYDROBROMIDE:Idiopathic pulmonary fibrosis:1.71), (EMPAGLIFLOZINLINAGLIPTIN:Idiopathic pulmonary fibrosis:0.34), (ESOMEPRAZOLE MAGNESIUMNAPROXEN:Idiopathic pulmonary fibrosis:0.4), (ETRETINATE:Idiopathic pulmonary fibrosis:0.1), (FERROUS CITRATE:Idiopathic pulmonary fibrosis:0.29), (FONDAPARINUX SODIUM:Idiopathic pulmonary fibrosis:0.04), (FUSIDIC ACID:Idiopathic pulmonary fibrosis:0.87), (HEPARIN CALCIUM:Idiopathic pulmonary fibrosis:0.46), (HERBALSTURMERIC:Idiopathic pulmonary fibrosis:0.49), (HYDROCODONE BITARTRATEIBUPROFEN:Idiopathic pulmonary fibrosis:0.29), (HYDROXYCHLOROQUINE DIPHOSPHATE:Idiopathic pulmonary fibrosis:0.49), (HYDROXYUREA:Idiopathic pulmonary fibrosis:0.19), (IMIPENEM:Idiopathic pulmonary fibrosis:0.14), (IMIPRAMINE HYDROCHLORIDE:Idiopathic pulmonary fibrosis:1.48), (INFLIXIMAB-DYYB:Idiopathic pulmonary fibrosis:0.13), (IRON SUCROSE:Idiopathic pulmonary fibrosis:0.35), (LEDIPASVIRSOFOSBUVIR:Idiopathic pulmonary fibrosis:0.77), (LEVOMEFOLATE CALCIUM:Idiopathic pulmonary fibrosis:0.25), (LIDOCAINEPRILOCAINE:Idiopathic pulmonary fibrosis:0.46), (LINEZOLID:Idiopathic pulmonary fibrosis:0.36), (LIOTHYRONINE SODIUM:Idiopathic pulmonary fibrosis:0.48), (LOMITAPIDE MESYLATE:Idiopathic pulmonary fibrosis:0.58), (MEPOLIZUMAB:Idiopathic pulmonary fibrosis:1.27), (METFORMIN HYDROCHLORIDEVILDAGLIPTIN:Idiopathic pulmonary fibrosis:0.37), (NILOTINIB:Idiopathic pulmonary fibrosis:1.23), (OLOPATADINE:Idiopathic pulmonary fibrosis:0.53), (ONABOTULINUMTOXINA:Idiopathic pulmonary fibrosis:0.19), (ORPHENADRINE:Idiopathic pulmonary fibrosis:0.32), (OXYBUTYNINOXYBUTYNIN CHLORIDE:Idiopathic pulmonary fibrosis:0.02), (PAMIDRONATE DISODIUM:Idiopathic pulmonary fibrosis:0.07), (PATIROMER:Idiopathic pulmonary fibrosis:0.44), (PEGINTERFERON BETA-1A:Idiopathic pulmonary fibrosis:0.36), (PEMETREXED DISODIUM:Idiopathic pulmonary fibrosis:0.69), (PENICILLIN:Idiopathic pulmonary fibrosis:1.01), (PENICILLIN G:Idiopathic pulmonary fibrosis:0.03), (PENTOSAN POLYSULFATE SODIUM:Idiopathic pulmonary fibrosis:0.37), (PHENOBARBITAL:Idiopathic pulmonary fibrosis:1.08), (PILOCARPINE:Idiopathic pulmonary fibrosis:0.49), (PILSICAINIDE HYDROCHLORIDE:Idiopathic pulmonary fibrosis:0.55), (POLYETHYLENE GLYCOL 4000:Idiopathic pulmonary fibrosis:0.21), (PONATINIB:Idiopathic pulmonary fibrosis:1.09), (PRAZOSIN HYDROCHLORIDE:Idiopathic pulmonary fibrosis:0.31), (PROTRIPTYLINE HYDROCHLORIDE:Idiopathic pulmonary fibrosis:0.07), (PYRAZINAMIDE:Idiopathic pulmonary fibrosis:0.66), (PYRIDOSTIGMINE BROMIDE:Idiopathic pulmonary fibrosis:0.76), (RETINOL:Idiopathic pulmonary fibrosis:0.19), (RIVASTIGMINE:Idiopathic pulmonary fibrosis:0.72), (SECUKINUMAB:Idiopathic pulmonary fibrosis:0.77), (SEVELAMER CARBONATE:Idiopathic pulmonary fibrosis:0.3), (SODIUM OXYBATE:Idiopathic pulmonary fibrosis:1.33), (SODIUM PHOSPHATE, DIBASICSODIUM PHOSPHATE, MONOBASIC:Idiopathic pulmonary fibrosis:0.1), (SOTALOL HYDROCHLORIDE:Idiopathic pulmonary fibrosis:0.3), (SUNITINIB MALATE:Idiopathic pulmonary fibrosis:0.51), (TAFLUPROST:Idiopathic pulmonary fibrosis:0.14), (TERBUTALINE:Idiopathic pulmonary fibrosis:0.16), (TERIFLUNOMIDE:Idiopathic pulmonary fibrosis:0.05), (TETRACYCLINE:Idiopathic pulmonary fibrosis:1.28), (THEOPHYLLINE:Idiopathic pulmonary fibrosis:0.23), (THYROID, PORCINE:Idiopathic pulmonary fibrosis:0.81), (TINZAPARIN SODIUM:Idiopathic pulmonary fibrosis:0.02), (TREPROSTINIL DIOLAMINE:Idiopathic pulmonary fibrosis:0.18), (VANCOMYCIN HYDROCHLORIDE:Idiopathic pulmonary fibrosis:0.54), (VARDENAFIL HYDROCHLORIDE TRIHYDRATE:Idiopathic pulmonary fibrosis:0.08), (ZILEUTON:Idiopathic pulmonary fibrosis:0.8), (ZIPRASIDONE HYDROCHLORIDEZIPRASIDONE MESYLATE:Idiopathic pulmonary fibrosis:0.05),(ARMODAFINIL:Idiopathic urticaria:0.07), (CALCIUM:Idiopathic urticaria:0.68), (CERTOLIZUMAB PEGOL:Idiopathic urticaria:0.13), (ENALAPRIL:Idiopathic urticaria:0.14), (EVOLOCUMAB:Idiopathic urticaria:0.32), (FERROUS SULFATEFERROUS SULFATE, DRIED:Idiopathic urticaria:0.11), (IBRUTINIB:Idiopathic urticaria:0.17), (MELATONIN:Idiopathic urticaria:0.1), (METRONIDAZOLE:Idiopathic urticaria:0.38), (POTASSIUM:Idiopathic urticaria:0.15), (RAMIPRIL:Idiopathic urticaria:0.12), (SODIUM OXYBATE:Idiopathic urticaria:0.36), (TAMSULOSIN:Idiopathic urticaria:0.01), (.ALPHA.1-PROTEINASE INHIBITOR HUMAN:Ill-defined disorder:0.51), (ACENOCOUMAROL:Ill-defined disorder:0.12), (ACETAMINOPHENOXYCODONE HYDROCHLORIDEOXYCODONE TEREPHTHALATE:Ill-defined disorder:0.64), (ACETAMINOPHENTRAMADOL:Ill-defined disorder:0.04), (ACETAZOLAMIDE:Ill-defined disorder:0.63), (ADO-TRASTUZUMAB EMTANSINE:Ill-defined disorder:0.35), (ALISKIREN HEMIFUMARATE:Ill-defined disorder:0.15), (AMANTADINE:Ill-defined disorder:0.5), (AMISULPRIDE:Ill-defined disorder:0.52), (AMLODIPINE BESYLATEATORVASTATIN CALCIUM:Ill-defined disorder:0.12), (AMLODIPINE BESYLATEHYDROCHLOROTHIAZIDEOLMESARTAN MEDOXOMIL:Ill-defined disorder:0.34), (AMMONIUM LACTATE:Ill-defined disorder:0.07), (APREPITANT:Ill-defined disorder:0.17), (ARIPIPRAZOLE LAUROXIL:Ill-defined disorder:0.11), (BENZTROPINE MESYLATE:Ill-defined disorder:0.12), (BETAMETHASONE DIPROPIONATECALCIPOTRIENECALCIPOTRIENE HYDRATE:Ill-defined disorder:0.07), (BORTEZOMIB:Ill-defined disorder:1.07), (BRINZOLAMIDE:Ill-defined disorder:0.03), (BROTIZOLAM:Ill-defined disorder:0.19), (BUDESONIDEFORMOTEROL FUMARATE:Ill-defined disorder:0.67), (CALCIPOTRIENE:Ill-defined disorder:0.13), (CALCITONIN:Ill-defined disorder:0.08), (CALCIUM CARBONATEVITAMIN D:Ill-defined disorder:0.47), (CANAGLIFLOZINMETFORMIN HYDROCHLORIDE:Ill-defined disorder:0.72), (CANAKINUMAB:Ill-defined disorder:0.47), (CARFILZOMIB:Ill-defined disorder:0.81), (CHLORPROMAZINE:Ill-defined disorder:0.23), (CHONDROITIN SULFATE (BOVINE)GLUCOSAMINE HYDROCHLORIDE:Ill-defined disorder:0.39), (CILASTATIN SODIUMIMIPENEM:Ill-defined disorder:0.1), (CLINDAMYCIN:Ill-defined disorder:0.73), (CODEINE PHOSPHATE:Ill-defined disorder:0.06), (CODEINE PHOSPHATEGUAIFENESIN:Ill-defined disorder:0.01), (CORTICOTROPIN:Ill-defined disorder:0.74), (CYPROHEPTADINE:Ill-defined disorder:0.09), (DACLIZUMAB:Ill-defined disorder:1.04), (DALTEPARIN:Ill-defined disorder:0.03), (DALTEPARIN SODIUM:Ill-defined disorder:0.09), (DANAZOL:Ill-defined disorder:0.42), (DARBEPOETIN ALFA:Ill-defined disorder:1.19), (DASABUVIROMBITASVIRPARITAPREVIRRITONAVIR:Ill-defined disorder:0.13), (DEFLAZACORT:Ill-defined disorder:0.61), (DUTASTERIDETAMSULOSIN HYDROCHLORIDE:Ill-defined disorder:0.32), (ELETRIPTAN HYDROBROMIDE:Ill-defined disorder:1.7), (EMPAGLIFLOZINLINAGLIPTIN:Ill-defined disorder:0.89), (ERYTHROMYCIN:Ill-defined disorder:0.32), (ESTRADIOLNORETHINDRONE ACETATE:Ill-defined disorder:0.07), (ETORICOXIB:Ill-defined disorder:0.21), (FLUOCINONIDE:Ill-defined disorder:0.72), (FORMOTEROL FUMARATEMOMETASONE FUROATE:Ill-defined disorder:0.6), (GLYCOPYRRONIUM:Ill-defined disorder:0.06), (GLYCOPYRRONIUMINDACATEROL:Ill-defined disorder:0.67), (HERBALSTURMERIC:Ill-defined disorder:0.57), (HYDROCORTISONE ACETATE:Ill-defined disorder:0.18), (HYDROXYCHLOROQUINE DIPHOSPHATE:Ill-defined disorder:0.56), (ILOPROST:Ill-defined disorder:0.79), (INDAPAMIDEPERINDOPRIL:Ill-defined disorder:0.43), (INDOMETHACIN:Ill-defined disorder:0.83), (INSULIN GLULISINE:Ill-defined disorder:0.62), (LACTOBACILLUS RHAMNOSUS:Ill-defined disorder:0.05), (LEDIPASVIRSOFOSBUVIR:Ill-defined disorder:1.64), (LERCANIDIPINE HYDROCHLORIDE:Ill-defined disorder:0.54), (LOMITAPIDE MESYLATE:Ill-defined disorder:0.64), (METFORMIN HYDROCHLORIDEROSIGLITAZONE MALEATE:Ill-defined disorder:0.09), (METHIMAZOLE:Ill-defined disorder:0.17), (METHYLPREDNISOLONE ACETATE:Ill-defined disorder:1.38), (MIDODRINE HYDROCHLORIDE:Ill-defined disorder:0.14), (MINOCYCLINE HYDROCHLORIDE:Ill-defined disorder:0.22), (MOXIFLOXACIN:Ill-defined disorder:0.08), (NALOXONE HYDROCHLORIDEOXYCODONE HYDROCHLORIDE:Ill-defined disorder:1.09), (NILOTINIB:Ill-defined disorder:1.38), (NINTEDANIB:Ill-defined disorder:0.45), (NIRAPARIB:Ill-defined disorder:0.17), (OCTREOTIDE ACETATE:Ill-defined disorder:0.07), (PANTOPRAZOLE MAGNESIUM:Ill-defined disorder:1.61), (PATIROMER:Ill-defined disorder:0.42), (PEMETREXED DISODIUM:Ill-defined disorder:0.36), (PIOGLITAZONE:Ill-defined disorder:0.14), (PITAVASTATIN CALCIUM:Ill-defined disorder:0.74), (PONATINIB:Ill-defined disorder:0.97), (POSACONAZOLE:Ill-defined disorder:0.24), (PREDNISOLONEPREDNISOLONE ACETATEPREDNISOLONE SODIUM SUCCINATE:Ill-defined disorder:0.46), (SACCHAROMYCES CEREVISIAE:Ill-defined disorder:0.14), (SACUBITRILVALSARTAN:Ill-defined disorder:1.03), (SELEXIPAG:Ill-defined disorder:0.07), (SULFAMETHOXAZOLE:Ill-defined disorder:0.5), (SULINDAC:Ill-defined disorder:0.02), (TERBINAFINE HYDROCHLORIDE:Ill-defined disorder:0.61), (TETRACYCLINE:Ill-defined disorder:1.28), (TIMOLOL:Ill-defined disorder:0.61), (TINZAPARIN SODIUM:Ill-defined disorder:0.03), (TOCOPHEROL:Ill-defined disorder:0.17), (TOLTERODINE:Ill-defined disorder:0.14), (TRASTUZUMAB:Ill-defined disorder:0.5), (TRIAMTERENE:Ill-defined disorder:0.83), (VALGANCICLOVIR HYDROCHLORIDE:Ill-defined disorder:0.21), (ZILEUTON:Ill-defined disorder:0.1), (GADOLINIUM:Imaging procedure:0.1), (ACETAMINOPHENCODEINE PHOSPHATE:Immune system disorder:1.23), (ACETAMINOPHENOXYCODONEOXYCODONE HYDROCHLORIDE:Immune system disorder:0.13), (AGALSIDASE BETA:Immune system disorder:0.13), (ALFACALCIDOL:Immune system disorder:0.01), (AMPHETAMINE ASPARTATEAMPHETAMINE SULFATEDEXTROAMPHETAMINE SACCHARATEDEXTROAMPHETAMINE SULFATE:Immune system disorder:1.38), (BENAZEPRILBENAZEPRIL HYDROCHLORIDE:Immune system disorder:0.09), (BICALUTAMIDE:Immune system disorder:0.16), (BISOPROLOL:Immune system disorder:0.14), (BUDESONIDEFORMOTEROL FUMARATE:Immune system disorder:0.17), (BUPRENORPHINE HYDROCHLORIDENALOXONE HYDROCHLORIDE:Immune system disorder:0.06), (CALCIUM CARBONATEVITAMIN D:Immune system disorder:0.59), (CANDESARTAN:Immune system disorder:0.54), (CANDESARTAN CILEXETIL:Immune system disorder:0.58), (CANDESARTAN CILEXETILHYDROCHLOROTHIAZIDE:Immune system disorder:0.05), (CARVEDILOL PHOSPHATE:Immune system disorder:0.57), (CEFTRIAXONE SODIUM:Immune system disorder:0.05), (CETIRIZINE HYDROCHLORIDEPSEUDOEPHEDRINE HYDROCHLORIDE:Immune system disorder:0.09), (CHOLESTYRAMINE:Immune system disorder:0.99), (CIPROFLOXACIN:Immune system disorder:0.5), (COLESEVELAM HYDROCHLORIDE:Immune system disorder:0.04), (DACLIZUMAB:Immune system disorder:0.61), (DAPAGLIFLOZIN PROPANEDIOL:Immune system disorder:0.26), (DASATINIB:Immune system disorder:0.81), (DIMENHYDRINATE:Immune system disorder:0.52), (DOCETAXEL:Immune system disorder:0.2), (DOXAZOSINDOXAZOSIN MESYLATE:Immune system disorder:0.24), (DRONABINOL:Immune system disorder:0.28), (DULAGLUTIDE:Immune system disorder:0.21), (DUTASTERIDE:Immune system disorder:0.08), (ELETRIPTAN HYDROBROMIDE:Immune system disorder:1.23), (ENALAPRIL MALEATE:Immune system disorder:0.73), (ENOXAPARIN:Immune system disorder:0.62), (ESOMEPRAZOLE MAGNESIUMNAPROXEN:Immune system disorder:0.18), (EVEROLIMUS:Immune system disorder:0.73), (FESOTERODINE FUMARATE:Immune system disorder:0.28), (FISH OIL\OMEGA-3 FATTY ACIDS:Immune system disorder:0.25), (FLECAINIDE:Immune system disorder:0.08), (FLUOCINONIDE:Immune system disorder:0.11), (FLUOXETINEFLUOXETINE HYDROCHLORIDE:Immune system disorder:0.29), (FORMOTEROL FUMARATEMOMETASONE FUROATE:Immune system disorder:0.16), (HYDROXYUREA:Immune system disorder:0.67), (HYOSCYAMINE:Immune system disorder:0.64), (ILOPROST:Immune system disorder:0.11), (IMIPRAMINE HYDROCHLORIDE:Immune system disorder:0.25), (INDAPAMIDE:Immune system disorder:0.11), (INDOMETHACIN:Immune system disorder:0.72), (ISOSORBIDE:Immune system disorder:0.73), (LEDIPASVIRSOFOSBUVIR:Immune system disorder:0.55), (LERCANIDIPINE:Immune system disorder:0.1), (LINAGLIPTIN:Immune system disorder:0.46), (LOMITAPIDE MESYLATE:Immune system disorder:0.3), (MECLIZINE HYDROCHLORIDE:Immune system disorder:0.07), (MEPOLIZUMAB:Immune system disorder:0.18), (METHENAMINE HIPPURATE:Immune system disorder:0.52), (METHYLPHENIDATE HYDROCHLORIDE:Immune system disorder:0.69), (METHYLPREDNISOLONE ACETATE:Immune system disorder:0.69), (NILOTINIB:Immune system disorder:0.88), (NINTEDANIB:Immune system disorder:0.07), (NIRAPARIB:Immune system disorder:0.05), (OXCARBAZEPINE:Immune system disorder:0.95), (PANTOPRAZOLE MAGNESIUM:Immune system disorder:0.94), (PATIROMER:Immune system disorder:0.05), (PAZOPANIB HYDROCHLORIDE:Immune system disorder:0.27), (PEGINTERFERON BETA-1A:Immune system disorder:0.91), (PIROXICAM:Immune system disorder:0.03), (PLANTAGO SEED:Immune system disorder:0.08), (PONATINIB:Immune system disorder:0.46), (PYRIDOXINE:Immune system disorder:0.02), (RALOXIFENE HYDROCHLORIDE:Immune system disorder:0.23), (RIZATRIPTAN:Immune system disorder:0.04), (SACUBITRILVALSARTAN:Immune system disorder:0.58), (SOLIFENACIN SUCCINATE:Immune system disorder:0.22), (TERBUTALINE SULFATE:Immune system disorder:0.36), (TERIFLUNOMIDE:Immune system disorder:1.14), (TETRACYCLINE:Immune system disorder:0.03), (TOCILIZUMAB:Immune system disorder:0.32), (TOLTERODINE TARTRATE:Immune system disorder:0.57), (ACETAMINOPHENOXYCODONEOXYCODONE HYDROCHLORIDE:Immune thrombocytopenic purpura:0.27), (ADO-TRASTUZUMAB EMTANSINE:Immune thrombocytopenic purpura:0.25), (AGALSIDASE BETA:Immune thrombocytopenic purpura:0.1), (ALCOHOL:Immune thrombocytopenic purpura:0.11), (AMANTADINE:Immune thrombocytopenic purpura:1.01), (AMISULPRIDE:Immune thrombocytopenic purpura:0.32), (AMPICILLIN:Immune thrombocytopenic purpura:0.68),(ARMODAFINIL:Immune thrombocytopenic purpura:0.72), (BENAZEPRILBENAZEPRIL HYDROCHLORIDE:Immune thrombocytopenic purpura:0.21), (BEPRIDIL:Immune thrombocytopenic purpura:0.64), (BERAPROST SODIUM:Immune thrombocytopenic purpura:0.87), (BETAHISTINE MESILATE:Immune thrombocytopenic purpura:0.03), (BETAMETHASONE DIPROPIONATE:Immune thrombocytopenic purpura:0.48), (BICALUTAMIDE:Immune thrombocytopenic purpura:0.2), (BISACODYL OR DOCUSATE SODIUM:Immune thrombocytopenic purpura:0.79), (BLINATUMOMAB:Immune thrombocytopenic purpura:0.22), (BUDESONIDEFORMOTEROL FUMARATE:Immune thrombocytopenic purpura:0.25), (BUPRENORPHINE HYDROCHLORIDE:Immune thrombocytopenic purpura:0.75), (BUPRENORPHINE HYDROCHLORIDENALOXONE HYDROCHLORIDE:Immune thrombocytopenic purpura:0.33), (BUSPIRONE HYDROCHLORIDE:Immune thrombocytopenic purpura:0.79), (CALCITONIN:Immune thrombocytopenic purpura:0.06), (CALCITONIN SALMON:Immune thrombocytopenic purpura:0.29), (CALCIUM ACETATE:Immune thrombocytopenic purpura:0.68), (CALCIUM CARBONATECHOLECALCIFEROLMAGNESIUM CARBONATE:Immune thrombocytopenic purpura:0.24), (CALCIUM CARBONATEVITAMIN D:Immune thrombocytopenic purpura:0.26), (CALCIUM LACTATE:Immune thrombocytopenic purpura:0.07), (CANAGLIFLOZINMETFORMIN HYDROCHLORIDE:Immune thrombocytopenic purpura:0.11), (CANDESARTAN CILEXETILHYDROCHLOROTHIAZIDE:Immune thrombocytopenic purpura:1.33), (CARISOPRODOL:Immune thrombocytopenic purpura:0.11), (CARVEDILOL PHOSPHATE:Immune thrombocytopenic purpura:0.85), (CEFAZOLIN:Immune thrombocytopenic purpura:0.35), (CERTOLIZUMAB PEGOL:Immune thrombocytopenic purpura:1.75), (CETUXIMAB:Immune thrombocytopenic purpura:0.42), (CHLORTHALIDONE:Immune thrombocytopenic purpura:0.47), (CHOLESTYRAMINE:Immune thrombocytopenic purpura:1.36), (CILASTATIN SODIUMIMIPENEM:Immune thrombocytopenic purpura:0.91), (CLINDAMYCIN:Immune thrombocytopenic purpura:0.93), (CLOZAPINE:Immune thrombocytopenic purpura:1.53), (CYPROHEPTADINE:Immune thrombocytopenic purpura:0.56), (DABIGATRAN:Immune thrombocytopenic purpura:0.48), (DACLIZUMAB:Immune thrombocytopenic purpura:0.62), (DALFAMPRIDINE:Immune thrombocytopenic purpura:0.62), (DEVICE:Immune thrombocytopenic purpura:0.46), (DIGITOXIN:Immune thrombocytopenic purpura:0.44), (DILAZEP:Immune thrombocytopenic purpura:0.01), (DIPHENIDOL HYDROCHLORIDE:Immune thrombocytopenic purpura:0.06), (DIPYRONE:Immune thrombocytopenic purpura:0.05), (DOCETAXEL:Immune thrombocytopenic purpura:0.14), (DOXAZOSINDOXAZOSIN MESYLATE:Immune thrombocytopenic purpura:0.99), (ECONAZOLE:Immune thrombocytopenic purpura:0.43), (EDOXABAN TOSYLATE:Immune thrombocytopenic purpura:1.9), (ELOTUZUMAB:Immune thrombocytopenic purpura:0.1), (EMPAGLIFLOZINLINAGLIPTIN:Immune thrombocytopenic purpura:0.25), (EPERISONE HYDROCHLORIDE:Immune thrombocytopenic purpura:0.04), (EPIRUBICIN:Immune thrombocytopenic purpura:0.57), (ETORICOXIB:Immune thrombocytopenic purpura:0.22), (EZETIMIBESIMVASTATIN:Immune thrombocytopenic purpura:0.43), (FENTANYL CITRATE:Immune thrombocytopenic purpura:0.02), (FERROUS SULFATEGLYCINE:Immune thrombocytopenic purpura:0.32), (FESOTERODINE FUMARATE:Immune thrombocytopenic purpura:0.61), (FLUDROCORTISONE ACETATE:Immune thrombocytopenic purpura:0.13), (FLUTICASONE FUROATE:Immune thrombocytopenic purpura:0.15), (FORMOTEROL:Immune thrombocytopenic purpura:0.12), (FORMOTEROL FUMARATE:Immune thrombocytopenic purpura:0.28), (FORMOTEROL FUMARATE DIHYDRATEMOMETASONE FUROATE:Immune thrombocytopenic purpura:0.41), (FOSPHENYTOINFOSPHENYTOIN SODIUM:Immune thrombocytopenic purpura:0.14), (FUSIDIC ACID:Immune thrombocytopenic purpura:0.68), (GIMERACILOTERACILTEGAFUR:Immune thrombocytopenic purpura:0.18), (GLYCOPYRRONIUMINDACATEROL:Immune thrombocytopenic purpura:0.26), (HYDROCHLOROTHIAZIDEIRBESARTAN:Immune thrombocytopenic purpura:1.12), (HYDROCORTISONE ACETATE:Immune thrombocytopenic purpura:0.23), (IBRITUMOMAB TIUXETAN:Immune thrombocytopenic purpura:0.38), (ICOSAPENT ETHYL:Immune thrombocytopenic purpura:0.07), (IMATINIB MESYLATE:Immune thrombocytopenic purpura:1.56), (INDOMETHACIN:Immune thrombocytopenic purpura:0.68), (INFLIXIMAB-DYYB:Immune thrombocytopenic purpura:0.13), (INSULIN DEGLUDEC:Immune thrombocytopenic purpura:0.07), (IRSOGLADINE MALEATE:Immune thrombocytopenic purpura:0.01), (LAFUTIDINE:Immune thrombocytopenic purpura:0.39), (LEVOLEUCOVORIN CALCIUM:Immune thrombocytopenic purpura:0.3), (LISDEXAMFETAMINE DIMESYLATE:Immune thrombocytopenic purpura:0.61), (LITHIUM:Immune thrombocytopenic purpura:0.11), (MEGESTROL ACETATE:Immune thrombocytopenic purpura:0.15), (METHENAMINE HIPPURATE:Immune thrombocytopenic purpura:0.26), (METHYLPREDNISOLONE ACETATE:Immune thrombocytopenic purpura:0.58), (METOLAZONE:Immune thrombocytopenic purpura:0.27), (MICAFUNGIN:Immune thrombocytopenic purpura:0.09), (MICONAZOLE NITRATE:Immune thrombocytopenic purpura:0), (MIDODRINE:Immune thrombocytopenic purpura:0.02), (MIGLUSTAT:Immune thrombocytopenic purpura:0.09), (MINOCYCLINEMINOCYCLINE HYDROCHLORIDE:Immune thrombocytopenic purpura:0.96), (MUPIROCINMUPIROCIN CALCIUM:Immune thrombocytopenic purpura:0.18), (NALOXONE HYDROCHLORIDEOXYCODONE HYDROCHLORIDE:Immune thrombocytopenic purpura:0.9), (NICOTINE:Immune thrombocytopenic purpura:0.18), (NINTEDANIB:Immune thrombocytopenic purpura:0.33), (OLODATEROL HYDROCHLORIDETIOTROPIUM BROMIDE MONOHYDRATE:Immune thrombocytopenic purpura:0.55), (OXYBUTYNIN CHLORIDE:Immune thrombocytopenic purpura:0.39), (PAMIDRONATE DISODIUM:Immune thrombocytopenic purpura:0.09), (PAROXETINE:Immune thrombocytopenic purpura:0.87), (PAZOPANIB HYDROCHLORIDE:Immune thrombocytopenic purpura:1.24), (PEGINTERFERON BETA-1A:Immune thrombocytopenic purpura:0.42), (PEMETREXED:Immune thrombocytopenic purpura:0.23), (PENICILLIN G:Immune thrombocytopenic purpura:0.11), (PIROXICAM:Immune thrombocytopenic purpura:0.06), (POLYETHYLENE GLYCOL 4000:Immune thrombocytopenic purpura:0.1), (PONATINIB:Immune thrombocytopenic purpura:1.04), (POTASSIUM CITRATETRISODIUM CITRATE DIHYDRATE:Immune thrombocytopenic purpura:0.24), (PRANLUKAST:Immune thrombocytopenic purpura:0.03), (PREDNISOLONE SODIUM SUCCINATE:Immune thrombocytopenic purpura:0.07), (PRIMIDONE:Immune thrombocytopenic purpura:0.86), (PROPAFENONE HYDROCHLORIDE:Immune thrombocytopenic purpura:0.06), (RALTEGRAVIR POTASSIUM:Immune thrombocytopenic purpura:0.88), (RAMUCIRUMAB:Immune thrombocytopenic purpura:0), (RANOLAZINE:Immune thrombocytopenic purpura:1.07), (REGORAFENIB:Immune thrombocytopenic purpura:0.09), (RIVASTIGMINE TARTRATE:Immune thrombocytopenic purpura:0.43), (ROFLUMILAST:Immune thrombocytopenic purpura:0.46), (ROPINIROLE HYDROCHLORIDE:Immune thrombocytopenic purpura:0.42), (SACUBITRILVALSARTAN:Immune thrombocytopenic purpura:0.78), (SARPOGRELATE:Immune thrombocytopenic purpura:0.62), (SCOPOLAMINE:Immune thrombocytopenic purpura:0.88), (SECUKINUMAB:Immune thrombocytopenic purpura:0.67), (SODIUM OXYBATE:Immune thrombocytopenic purpura:0.71), (TESTOSTERONE UNDECANOATE:Immune thrombocytopenic purpura:0.04), (TETRACYCLINE:Immune thrombocytopenic purpura:0.23), (TINZAPARIN SODIUM:Immune thrombocytopenic purpura:0.65), (TIOTROPIUM:Immune thrombocytopenic purpura:0.53), (TIZANIDINE HYDROCHLORIDE:Immune thrombocytopenic purpura:0.76), (TOLTERODINE TARTRATE:Immune thrombocytopenic purpura:0.43), (TROSPIUM CHLORIDE:Immune thrombocytopenic purpura:0.09), (UMECLIDINIUM BROMIDEVILANTEROL TRIFENATATE:Immune thrombocytopenic purpura:0.29), (URAPIDIL:Immune thrombocytopenic purpura:0.04), (VITAMIN B:Immune thrombocytopenic purpura:0.86), (ZINC:Immune thrombocytopenic purpura:0.07), (.ALPHA.1-PROTEINASE INHIBITOR HUMAN:Immunisation:0.2), (ACETAMINOPHENOXYCODONEOXYCODONE HYDROCHLORIDE:Immunisation:0.07), (ACETAZOLAMIDE:Immunisation:0.24), (ACLIDIN IUM BROMIDE:Immunisation:0.04), (ADO-TRASTUZUMAB EMTANSINE:Immunisation:0.11), (AGALSIDASE BETA:Immunisation:0.19), (ALCOHOL:Immunisation:0.15), (AMANTADINE:Immunisation:0. 26), (AMIODARONE HYDROCHLORIDE:Immunisation:0.66), (AMPICILLIN:Immunisation:0.92), (ANASTROZOL E:Immunisation:1.72), (BENAZEPRIL HYDROCHLORIDE:Immunisation:0.45), (BISACODYL OR DOCUSATE SODIUM:Immunisation:0.85), (BISOPROLOL FUMARATE:Immunisation:0.83), (BUMETANIDE:Immunisation:1.29), (BUPRENORPHINE HYDROCHLORIDENALOXONE HYDROCHLORIDE:Immunisation:0.21), (BUPROPION:Immunisation:0.71), (BUTYLSCOPO LAMINE BROMIDE:Immunisation:0.29), (CALCITONIN SALMON:Immunisation:0.12), (CALCIUM ACETATE:Immunisation:0.32), (CARFILZOMIB:Immunisation:0.77), (CEFEPIME HYDROCHLORIDE:Immunisation:0.03), (CLOBETASOL:Immunisation:0.01), (DACLIZUMA B:Immunisation:0.66), (DARATUMUMAB:Immunisation:0.05), (DOXEPIN HYDROCHLORIDE:Immunisation:0.13), (DRONABINOL:Immunisation:0.16), (EDOXABAN TOSYLATE:Immunisation:0.32), (ELTROMBOPAG OLAMINE:Immunisation:0.03), (ESTROGENS, CONJUGATEDMEDROXYPROGESTERONE ACETATE:Immunisation:0.23), (FEBUXOSTAT:Immunisation:0.48), (FORMOTEROL FUMARATE DIHYDRATEMOMETASONE FUROATE:Immunisation:0.31), (FORMOTEROL FUMARATEMOMETASONE FUROATE:Immunisation:0.17), (GLYCOPYRRONIUMINDACATEROL:Immunisation:0.13), (GUAIFENESIN:Immunisation:0.36), (HYDROCHLOROTHIAZIDEIRBESARTAN:Immunisa tion:0.37), (HYDROXYZINE PAMOATE:Immunisation:1.31), (HYOSCYAMINE:Immunisation:0), (ILOPROST:Immunisatio n:0.43), (IMIPRAMINE HYDROCHLORIDE:Immunisation:0.21), (INDAPAMIDEPERINDOPRIL:Immunisation:0.41), (ITRACONAZOLE:Immunisation:0.18), (LERCANIDIPINE HYDROCHLORIDE:Immunisation:0.4), (LEVALBUTEROL HYDROCHLORIDE:Immunisation:0.82), (MEPOLIZUMAB:Immunisation:0.01), (METHENA MINE HIPPURATE:Immunisation:0.41), (METHIMAZOLE:Immunisation:0.06), (METHYLPREDNIS OLONE ACETATE:Immunisation:1.31), (MOXIFLOXACIN:Immunisation:0.31), (NADOLOL:Immunisa tion:0.02), (NALOXONE HYDROCHLORIDEOXYCODONE HYDROCHLORIDE:Immunisation:0.33), (NORTRIPTYLINE:Immunisation:0.51), (OLODATE ROL HYDROCHLORIDETIOTROPIUM BROMIDE MONOHYDRATE:Immunisation:0.09), (ONABOTULINUMTOXINA:Immunisation:0.01), (PA CLITAXEL:Immunisation:0.66), (PAROXETINE:Immunisation:0.82), (PEGINTERFERON BETA-1A:Immunisation:0.54), (PHENOBARBITAL:Immunisation:1.03), (PHENYTOIN SODIUM:Immunisation:0.34), (POLYETHYLENE GLYCOLS:Immunisation:1.19), (PONATINIB:Immunisation:0.19), (PRAMIPEXOLE:Immunisa tion:0.41), (PROPRANOLOLPROPRANOLOL HYDROCHLORIDE:Immunisation:0.08), (PYRAZINAMIDE:Immunisation:0.74), (RALOXIFE NE HYDROCHLORIDE:Immunisation:0.09), (RANITIDINE:Immunisation:0.55), (RANOLAZINE: Immunisation:0.72), (ROSUVASTATIN:Immunisation:0.15), (SCOPOLAMINE:Immunisation:0. 03), (TINZAPARIN SODIUM:Immunisation:0.06), (TIOTROPIUM:Immunisation:0.13), (TOLTERODINE TARTRATE:Immunisation:0.35), (TRASTUZUMAB:Immunisation:0.49), (VINCRISTINE SULFATE:Immunisation:0.17), (VITAMIN A:Immunisation:0.2), (ZINC:Immunisation:0.07), (ACETAMINOPHENOXYCODONEOXYC ODONE HYDROCHLORIDE:Immunodeficiency:0.17), (ACETAZOLAMIDE:Immunodeficiency:0.35), (ACLIDINIUM BROMIDE:Immunodeficiency:0.12), (AMPICILLIN:Immunodeficiency:0.2), (ANASTROZOLE:Immunodeficiency:0.65),(ARMODAFINIL:Immunodeficiency:0.99), (BENAZEPRILBENAZE PRIL HYDROCHLORIDE:Immunodeficiency:0.07), (BUPRENORPHINE HYDROCHLORIDE:Immunodeficiency:0.32), (BUPRENORPHINE HYDROCHLORIDENALOXONE HYDROCHLORIDE:Immunodeficiency:0.16), (CALCIUM ACETATE:Immunodeficiency:0.33), (CANAKINUMAB:Immunodeficiency:0.44), (CANDESA RTAN CILEXETILHYDROCHLOROTHIAZIDE:Immunodeficiency:0.47), (CARVEDILOL PHOSPHATE:Immunodeficiency:0.41), (CERTOLIZUMAB PEGOL:Immunodeficiency:1.34), (CHONDROITIN SULFATE (BOVINE)GLUCOSAMINE HYDROCHLORIDE:Immunodeficiency:0.09), (CILASTATIN SODIUMIMIPENEM:Immunodeficiency:0.15), (CINACALCET HYDROCHLORIDE:Immunodeficiency:0.14), (CLOZAPINE:Immunodeficiency:0.26), (COD LIVER OIL:Immunodeficiency:0.08), (DACLIZUMAB:Immunodeficiency:0.72), (DALFAMPRIDINE:I mmunodeficiency:0.91), (DAPAGLIFLOZIN PROPANEDIOL:Immunodeficiency:0.09), (DIPYRONE:Immunodeficiency:0.19), (DOCUSATE:Immunodeficiency:0.48), (EDOXABAN TOSYLATE:Immunodeficiency:0.05), (ELTROMBOPAG OLAMINE:Immunodeficiency:0.12), (ERYTHROPOIETIN:Immunodeficiency:0.49), (ESOMEP RAZOLE MAGNESIUMNAPROXEN:Immunodeficiency:0.21), (ESTROGENS, CONJUGATEDMEDROXYPROGESTERONE ACETATE:Immunodeficiency:0.25), (EVOLOCUMAB:Immunodeficiency:1.13), (FISH OIL\OMEGA-3 FATTY ACIDS:Immunodeficiency:0.06), (FLECAINIDE:Immunodeficiency:0.55), (FLUOCINONIDE:I mmunodeficiency:0.17), (FLUTICASONE FUROATEVILANTEROL TRIFENATATE:Immunodeficiency:1.01), (FORMOTEROL FUMARATEMOMETASONE FUROATE:Immunodeficiency:0.72), (GLICLAZIDE:Immunodeficiency:0.12), (GLYBURIDE:I mmunodeficiency:0.3), (GLYCOPYRRONIUM:Immunodeficiency:0.19), (GLYCOPYRRONIU MINDACATEROL:Immunodeficiency:0.36), (HYDROCHLOROTHIAZIDEIRBESARTAN:I mmunodeficiency:0.47), (HYDROCHLOROTHIAZIDELOSARTAN POTASSIUM:Immunodeficiency:0.28), (HYDROCHLOROTHIAZIDEVALSARTAN:Immuno deficiency:0.54), (HYDROXYZINE PAMOATE:Immunodeficiency:1.18), (HYOSCYAMINE:Immunodeficiency:0.39), (ILOPROST: Immunodeficiency:0.21), (IMIPRAMINE HYDROCHLORIDE:Immunodeficiency:0.32), (INDAPAMIDE:Immunodeficiency:0.15), (IND OMETHACIN:Immunodeficiency:0.41), (LABETALOL HYDROCHLORIDE:Immunodeficiency:0.06), (LABETALOLLABETALOL HYDROCHLORIDE:Immunodeficiency:0.84), (LACTASE:Immunodeficiency:0.16), (LATANO PROST:Immunodeficiency:0.7), (LERCANIDIPINE:Immunodeficiency:0.41), (LERCANIDIPIN E HYDROCHLORIDE:Immunodeficiency:0.25), (MEPOLIZUMAB:Immunodeficiency:0.62), (M ETHENAMINE HIPPURATE:Immunodeficiency:0.33), (METHYLPREDNISOLONE ACETATE:Immunodeficiency:1.14), (METOLAZONE:Immunodeficiency:0.04), (METRONIDA ZOLEMETRONIDAZOLE HYDROCHLORIDE:Immunodeficiency:0.77), (MODAFINIL:Immunodeficiency:0.72), (MOXI FLOXACIN:Immunodeficiency:0.08), (NILOTINIB:Immunodeficiency:0.63), (NINTEDANIB:I mmunodeficiency:0), (NORETHINDRONE:Immunodeficiency:0.03), (OLANZAPINE:Immunod eficiency:0.92), (PANTOPRAZOLE MAGNESIUM:Immunodeficiency:1.52), (PAROXETINEPAROXETINE HYDROCHLORIDE:Immunodeficiency:0.6), (PAZOPANIB HYDROCHLORIDE:Immunodeficiency:0.01), (PEGINTERFERON BETA-1A:Immunodeficiency:0.44), (PERINDOPRIL:Immunodeficiency:0.04), (PONATINIB:Immunod eficiency:0.21), (PRAVASTATIN SODIUM:Immunodeficiency:1.47), (PYRAZINAMIDE:Immunodeficiency:0.67), (PYRIDOXIN E:Immunodeficiency:0.11), (RABEPRAZOLE:Immunodeficiency:0.49), (RALOXIFENE HYDROCHLORIDE:Immunodeficiency:0.33), (ROSUVASTATIN:Immunodeficiency:0.04), (S ACUBITRILVALSARTAN:Immunodeficiency:0.33), (SCOPOLAMINE:Immunodeficiency:0.0 6), (SECUKINUMAB:Immunodeficiency:0.76), (SOTALOL:Immunodeficiency:0.57), (SUMAT RIPTANSUMATRIPTAN SUCCINATE:Immunodeficiency:1.27), (THALIDOMIDE:Immunodeficiency:0.15), (TIMOLOL: Immunodeficiency:0.12), (TIOTROPIUM:Immunodeficiency:0.2), (TOCILIZUMAB:Immunodef iciency:0.6), (TRIAMTERENE:Immunodeficiency:0.41), (UMECLIDINIUM BROMIDE:Immunodeficiency:0), (VILAZODONE HYDROCHLORIDE:Immunodeficiency:0.09), (VITAMIN A:Immunodeficiency:0.27), (ZAFIRLUKAST:Immunodeficiency:0.09), (ZINC:Immunodeficienc y:0.38), (ACETAMINOPHENDIPHENHYDRAMINE:Immunodeficiency common variable:0.45), (ACETAMINOPHENDIPHENHYDRAMINE HYDROCHLORIDE:Immunodeficiency common variable:0.13), (ACETAMINOPHENPROPOXYPHENE HYDROCHLORIDE:Immunodeficiency common variable:0.64), (ACETAMINOPHENPROPOXYPHENE NAPSYLATE:Immunodeficiency common variable:1.05), (ALCOHOL:Immunodeficiency common variable:0.25), (ALEMTUZUMAB:Immunodeficiency common variable:0.53), (ALENDRONIC ACID:Immunodeficiency common variable:0.71), (ALFACALCIDOL:Immunodeficiency common variable:0.05), (AMANTADINE HYDROCHLORIDE:Immunodeficiency common variable:0.42), (AMLODIPINE BESYLATEATORVASTATIN CALCIUM:Immunodeficiency common variable:0.08), (AMLODIPINE BESYLATEHYDROCHLOROTHIAZIDEOLMESARTAN MEDOXOMIL:Immunodeficiency common variable:0.33), (ASCORBIC ACID/CHROMIUM/SELENIUM/VANADYL SULFATE:Immunodeficiency common variable:0.29), (ASPIRINCAFFEINESALICYLAMIDE:Immunodeficiency common variable:0.26), (ASPIRINDIPYRIDAMOLE:Immunodeficiency common variable:0.02), (ATROPINEDIPHENOXYLATE:Immunodeficiency common variable:0.14), (AZTREONAM:Immunodeficiency common variable:0.2), (BALSALAZIDE DISODIUM:Immunodeficiency common variable:0.6), (BETAMETHASONE DIPROPIONATECALCIPOTRIENECALCIPOTRIENE HYDRATE:Immunodeficiency common variable:0.98), (BETAMETHASONECALCIPOTRIENE:Immunodeficiency common variable:0.58), (BROMAZEPAM:Immunodeficiency common variable:0.11), (BUDESONIDEFORMOTEROL FUMARATE:Immunodeficiency common variable:1.1), (BUPRENORPHINENALOXONE:Immunodeficiency common variable:0.13), (BUTYLSCOPOLAMINE BROMIDE:Immunodeficiency common variable:0.35), (CALCIUM CARBONATEERGOCALCIFEROLRETINOL:Immunodeficiency common variable:0.02), (CANAGLIFLOZINMETFORMIN HYDROCHLORIDE:Immunodeficiency common variable:0.68), (CANDESARTAN CILEXETILHYDROCHLOROTHIAZIDE:Immunodeficiency common variable:1.19), (CARBIDOPA:Immunodeficiency common variable:0.01), (CARFILZOMIB:Immunodeficiency common variable:0.83), (CASANTHRANOLDOCUSATE SODIUM:Immunodeficiency common variable:0.27), (CEFAZOLIN:Immunodeficiency common variable:0.81), (CEFOTAXIME SODIUM:Immunodeficiency common variable:0.16), (CERIVASTATIN SODIUM:Immunodeficiency common variable:0.13), (CHLOROTHIAZIDE:Immunodeficiency common variable:0.48), (CILASTATIN SODIUMIMIPENEM:Immunodeficiency common variable:0.51), (CINACALCET HYDROCHLORIDE:Immunodeficiency common variable:0.67), (CLOMIPRAMINE HYDROCHLORIDE:Immunodeficiency common variable:0.12), (CLOSTRIDIUM TETANI TOXOID ANTIGEN (FORMALDEHYDE INACTIVATED):Immunodeficiency common variable:0.02), (CODEINE PHOSPHATE:Immunodeficiency common variable:0.16), (COLLAGEN:Immunodeficiency common variable:0.04), (CORTICOTROPIN:Immunodeficiency common variable:0.9), (DABIGATRAN:Immunodeficiency common variable:0.01), (DACLIZUMAB:Immunodeficiency common variable:1.47), (DALTEPARIN:Immunodeficiency common variable:0.31), (DANAZOL:Immunodeficiency common variable:0.64), (DAPAGLIFLOZIN PROPANEDIOL:Immunodeficiency common variable:0.91), (DAPSONE:Immunodeficiency common variable:0.54), (DARBEPOETIN ALFA:Immunodeficiency common variable:0.44), (DASATINIB:Immunodeficiency common variable:0.88), (DEXPANTHENOLNIACINAMIDEPYRIDOXINE HYDROCHLORIDERIBOFLAVIN 5′-PHOSPHATE SODIUMTHIAMINE HYDROCHLORIDE:Immunodeficiency common variable:0.08), (DEXTROMETHORPHAN HYDROBROMIDEQUINIDINE SULFATE:Immunodeficiency common variable:0), (DICLOFENAC SODIUMMISOPROSTOL:Immunodeficiency common variable:0.3), (DIPYRONE:Immunodeficiency common variable:0.68), (DIPYRONEPROMETHAZINE HYDROCHLORIDE:Immunodeficiency common variable:0.23), (DORZOLAMIDE HYDROCHLORIDE:Immunodeficiency common variable:0.6), (DUTASTERIDETAMSULOSIN HYDROCHLORIDE:Immunodeficiency common variable:0.39), (EDOXABAN TOSYLATE:Immunodeficiency common variable:0.76), (EMPAGLIFLOZINLINAGLIPTIN:Immunodeficiency common variable:0.8), (EPHEDRINE HYDROCHLORIDEGUAIFENESIN:Immunodeficiency common variable:0.3), (ESOMEPRAZOLE MAGNESIUMNAPROXEN:Immunodeficiency common variable:0.65), (ESTRADIOL BENZOATE:Immunodeficiency common variable:0.16), (ETHACRYNIC ACID:Immunodeficiency common variable:0.15), (ETHINYL ESTRADIOLNORETHINDRONE:Immunodeficiency common variable:0.66), (ETORICOXIB:Immunodeficiency common variable:0.41), (EVOLOCUMAB:Immunodeficiency common variable:1.72), (FENOTEROL HYDROBROMIDE:Immunodeficiency common variable:0.24), (FERROUS BISGLYCINATE:Immunodeficiency common variable:0.11), (FERROUS SULFATE ANHYDROUS:Immunodeficiency common variable:0.09), (FINGOLIMOD HYDROCHLORIDE:Immunodeficiency common variable:0.07), (FOLATE SODIUM:Immunodeficiency common variable:0.69), (FORMOTEROL FUMARATEMOMETASONE FUROATE:Immunodeficiency common variable:1.24), (FUSIDIC ACID:Immunodeficiency common variable:0.72), (GLICLAZIDE:Immunodeficiency common variable:0.49), (GLIPIZIDEMETFORMIN HYDROCHLORIDE:Immunodeficiency common variable:0.13), (GLYCOPYRROLATEINDACATEROL MALEATE:Immunodeficiency common variable:0.21), (GLYCOPYRRONIUMINDACATEROL:Immunodeficiency common variable:0.68), (HALOPERIDOL:Immunodeficiency common variable:0.52), (HYDROCHLOROTHIAZIDETELMISARTAN:Immunodeficiency common variable:0.44), (ILOPROST:Immunodeficiency common variable:0.66), (IMIPENEM:Immunodeficiency common variable:0.02), (IMIPRAMINE PAMOATE:Immunodeficiency common variable:0), (INDACATEROL:Immunodeficiency common variable:0.24), (INDAPAMIDEPERINDOPRIL:Immunodeficiency common variable:0.13), (INFLIXIMAB-DYYB:Immunodeficiency common variable:0.49), (INSULIN BEEF:Immunodeficiency common variable:0.34), (INTERFERON:Immunodeficiency common variable:0.09), (INTERFERON GAMMA-1B:Immunodeficiency common variable:0.93), (IRONVITAMINS:Immunodeficiency common variable:0.56), (ISOPROPYL ALCOHOLLEUPROLIDE ACETATENORETHINDRONE ACETATE:Immunodeficiency common variable:0.09), (ITRACONAZOLESODIUM CHLORIDE:Immunodeficiency common variable:0.19), (KETOPROFEN:Immunodeficiency common variable:0.05), (LEDIPASVIRSOFOSBUVIR:Immunodeficiency common variable:0.48), (LENALIDOMIDE:Immunodeficiency common variable:1.09), (LOMITAPIDE MESYLATE:Immunodeficiency common variable:0.76), (MEPOLIZUMAB:Immunodeficiency common variable:0.99), (METHENAMINE MANDELATE:Immunodeficiency common variable:0.34), (MORPHINE SULFATENALTREXONE HYDROCHLORIDE:Immunodeficiency common variable:0.07), (NALOXEGOL OXALATE:Immunodeficiency common variable:0.22), (NALOXONE HYDROCHLORIDEOXYCODONE HYDROCHLORIDE:Immunodeficiency common variable:0.88), (NILOTINIB:Immunodeficiency common variable:0.82), (NINTEDANIB:Immunodeficiency common variable:0.41), (OSELTAMIVIR:Immunodeficiency common variable:0.37), (OXYBUTYNINOXYBUTYNIN CHLORIDE:Immunodeficiency common variable:0.31), (PANTOPRAZOLE MAGNESIUM:Immunodeficiency common variable:2), (PAPAYA:Immunodeficiency common variable:0.26), (PATIROMER:Immunodeficiency common variable:0.43), (PEGINTERFERON BETA-1A:Immunodeficiency common variable:0.8), (PENICILLIN V:Immunodeficiency common variable:0.5), (PHENTERMINE HYDROCHLORIDE:Immunodeficiency common variable:0.28), (PHENYTOIN SODIUM:Immunodeficiency common variable:0.51), (PONATINIB:Immunodeficiency common variable:1.34), (PREDNISOLONE SODIUM PHOSPHATE:Immunodeficiency common variable:0.78), (PROTRIPTYLINE HYDROCHLORIDE:Immunodeficiency common variable:1.21), (PYRAZINAMIDE:Immunodeficiency common variable:0.71), (SALMETEROL:Immunodeficiency common variable:0.37), (SECUKINUMAB:Immunodeficiency common variable:0.87), (SELEGILINE:Immunodeficiency common variable:0.75), (SIBUTRAMINE HYDROCHLORIDE:Immunodeficiency common variable:0.17), (SOFOSBUVIRVELPATASVIR:Immunodeficiency common variable:0.09), (SUNITINIB MALATE:Immunodeficiency common variable:0.03), (TASIMELTEON:Immunodeficiency common variable:0.52), (TEDUGLUTIDEWATER:Immunodeficiency common variable:0.55), (TERBUTALINE SULFATE:Immunodeficiency common variable:1.68), (TERIFLUNOMIDE:Immunodeficiency common variable:0.87), (TETRABENAZINE:Immunodeficiency common variable:0.02), (TIBOLONE:Immunodeficiency common variable:0.22), (TICAGRELOR:Immunodeficiency common variable:0.26), (VARDENAFIL HYDROCHLORIDE TRIHYDRATE:Immunodeficiency common variable:0.06), (YEAST:Immunodeficiency common variable:0.51), (ZICONOTIDE ACETATE:Immunodeficiency common variable:0.18), (.ALPHA.1-PROTEINASE INHIBITOR HUMAN:Immunosuppressant drug therapy:0.44), (ABACAVIR SULFATEDOLUTEGRAVIR SODIUMLAMIVUDINE:Immunosuppressant drug therapy:0.05), (ACETAMINOPHENOXYCODONEOXYCODONE HYDROCHLORIDE:Immunosuppressant drug therapy:0.45), (ACETAMINOPHENPROPOXYPHENE HYDROCHLORIDE:Immunosuppressant drug therapy:0.2), (ADO-TRASTUZUMAB EMTANSINE:Immunosuppressant drug therapy:0.21), (ALENDRONIC ACID:Immunosuppressant drugtherapy:0.05), (AMINO ACIDSDEXTROSEELECTROLYTES NOSSOYBEAN OILVITAMINS:Immunosuppressant drug therapy:0.21), (AMINOSALICYLIC ACID:Immunosuppressant drug therapy:0.3), (AMISULPRIDE:Immunosuppressant drug therapy:0.07), (AMITRIPTYLINE HYDROCHLORIDE:Immunosuppressant drug therapy:0.24), (AMLODIPINEVALSARTAN:Immunosuppressant drug therapy:0.26), (AMPHETAMINE ASPARTATEAMPHETAMINE SULFATEDEXTROAMPHETAMINE SACCHARATEDEXTROAMPHETAMINE SULFATE:Immunosuppressant drug therapy:0.52),(ARMODAFINIL:Immunosuppressant drug therapy:0.27), (ASPIRINDIPYRIDAMOLE:Immunosuppressant drug therapy:0.56), (ASUNAPREVIR:Immunosuppressant drug therapy:0.33), (AVOBENZONEHOMOSALATEOCTISALATE:Immunosuppressant drug therapy:0.3), (BENDAMUSTINE HYDROCHLORIDE:Immunosuppressant drug therapy:0.61), (BISACODYL OR DOCUSATE SODIUM:Immunosuppressant drug therapy:1.06), (BLINATUMOMAB:Immunosuppressant drug therapy:1.04), (BUDESONIDEFORMOTEROL FUMARATE:Immunosuppressant drug therapy:0.44), (BUPRENORPHINE HYDROCHLORIDE:Immunosuppressant drug therapy:0.23), (CALCIUM CARBONATEERGOCALCIFEROLRETINOL:Immunosuppressant drug therapy:0.14), (CALCIUM CARBONATEVITAMIN D:Immunosuppressant drug therapy:0.13), (CANAGLIFLOZIN:Immunosuppressant drug therapy:0.08), (CANAGLIFLOZINMETFORMIN HYDROCHLORIDE:Immunosuppressant drug therapy:0.3), (CANDESARTAN CILEXETILHYDROCHLOROTHIAZIDE:Immunosuppressant drug therapy:1.16), (CARFILZOMIB:Immunosuppressant drug therapy:0.75), (CARVEDILOL PHOSPHATE:Immunosuppressant drug therapy:0.59), (CEFOPERAZONE SODIUMSULBACTAM:Immunosuppressant drug therapy:0.04), (CEFOTAXIMECEFOTAXIME SODIUM:Immunosuppressant drug therapy:0.09), (CEFPODOXIME PROXETIL:Immunosuppressant drug therapy:0.06), (CHLORAMBUCIL:Immunosuppressant drug therapy:0.63), (CHLOROQUINE PHOSPHATE:Immunosuppressant drug therapy:0.5), (CHLOROTHIAZIDE:Immunosuppressant drug therapy:0.13), (CHOLESTYRAMINE:Immunosuppressant drug therapy:1.05), (CICLESONIDE:Immunosuppressant drug therapy:0.38), (CLINDAMYCIN PHOSPHATE:Immunosuppressant drug therapy:0.07), (CLOZAPINE:Immunosuppressant drug therapy:1.05), (DABIGATRAN:Immunosuppressant drug therapy:0.24), (DARATUMUMAB:Immunosuppressant drug therapy:0.16), (DESFLURANE:Immunosuppressant drug therapy:0.46), (DESLORATADINE:Immunosuppressant drug therapy:0.78), (DESVENLAFAXINE SUCCINATE:Immunosuppressant drug therapy:0.13), (DEXAMETHASONE ACETATE:Immunosuppressant drug therapy:0.51), (DIMETHICONESILICON DIOXIDESIMETHICONE:Immunosuppressant drug therapy:0.09), (DOCUSATE CALCIUM:Immunosuppressant drug therapy:0.17), (DOFETILIDE:Immunosuppressant drug therapy:0.11), (DRONABINOL:Immunosuppressant drug therapy:0.6), (DRONEDARONE:Immunosuppressant drug therapy:0.04), (EDOXABAN TOSYLATE:Immunosuppressant drug therapy:0.66), (ELOTUZUMAB:Immunosuppressant drug therapy:0.87), (ELTROMBOPAG OLAMINE:Immunosuppressant drug therapy:0.11), (ETORICOXIB:Immunosuppressant drug therapy:0.68), (EXEMESTANE:Immunosuppressant drug therapy:0.2), (FENOTEROL HYDROBROMIDEIPRATROPIUM BROMIDE:Immunosuppressant drug therapy:0), (FERROUS FUMARATE:Immunosuppressant drug therapy:0.24), (FERROUS SULFATEGLYCINE:Immunosuppressant drug therapy:0.21), (FESOTERODINE FUMARATE:Immunosuppressant drug therapy:0.17), (FLECAINIDE:Immunosuppressant drug therapy:0.96), (FLUOXETINEFLUOXETINE HYDROCHLORIDE:Immunosuppressant drug therapy:0.36), (FLUTICASONE FUROATE:Immunosuppressant drug therapy:0.17), (FLUTICASONE FUROATESALMETEROL XINAFOATE:Immunosuppressant drug therapy:0.58), (FORMOTEROL:Immunosuppressant drug therapy:0.02), (FORMOTEROL FUMARATEMOMETASONE FUROATE:Immunosuppressant drug therapy:0.17), (FOSPHENYTOINFOSPHENYTOIN SODIUM:Immunosuppressant drug therapy:0.06), (FULVESTRANT:Immunosuppressant drug therapy:0.46), (FUSIDIC ACID:Immunosuppressant drug therapy:0.18), (GLYCOPYRROLATE:Immunosuppressant drug therapy:0.48), (GLYCOPYRRONIUMINDACATEROL:Immunosuppressant drug therapy:0.56), (HUMAN PLATELET, ALLOGENIC:Immunosuppressant drug therapy:0.09), (HYDROCHLOROTHIAZIDELISINOPRIL:Immunosuppressant drug therapy:0.84), (HYDROCHLOROTHIAZIDELOSARTAN POTASSIUM:Immunosuppressant drug therapy:0.46), (HYDROCHLOROTHIAZIDETELMISARTAN:Immunosuppressant drug therapy:0.15), (HYDROXYCHLOROQUINE DIPHOSPHATE:Immunosuppressant drug therapy:0.56), (HYDROXYZINE PAMOATE:Immunosuppressant drug therapy:0.57), (IBRITUMOMAB TIUXETAN:Immunosuppressant drug therapy:0.78), (IDELALISIB:Immunosuppressant drug therapy:0.33), (ILOPROST:Immunosuppressant drug therapy:0.46), (IMIPRAMINE HYDROCHLORIDE:Immunosuppressant drug therapy:0.3), (INSULIN DEGLUDEC:Immunosuppressant drug therapy:0.08), (IRINOTECAN HYDROCHLORIDE:Immunosuppressant drug therapy:0.07), (ISOFLURANE:Immunosuppressant drug therapy:0.36), (LACOSAMIDE:Immunosuppressant drug therapy:0.38), (LERCANIDIPINE HYDROCHLORIDE:Immunosuppressant drug therapy:1.06), (LEVOLEUCOVORIN CALCIUM:Immunosuppressant drug therapy:0.45), (LEVOTHYROXINE SODIUMPOTASSIUM IODIDE:Immunosuppressant drug therapy:0.07), (LISDEXAMFETAMINE DIMESYLATE:Immunosuppressant drug therapy:0.16), (MEGESTROL ACETATE:Immunosuppressant drug therapy:0.31), (METHENOLONE ENANTHATE:Immunosuppressant drug therapy:0.27), (METHIMAZOLE:Immunosuppressant drug therapy:0.58), (METHOCARBAMOL:Immunosuppressant drug therapy:0.08), (METILDIGOXIN:Immunosuppressant drug therapy:0.42), (MUPIROCINMUPIROCIN CALCIUM:Immunosuppressant drug therapy:0.33), (NALOXONE:Immunosuppressant drug therapy:0.05), (NALOXONE HYDROCHLORIDEOXYCODONE HYDROCHLORIDE:Immunosuppressant drug therapy:0.57), (NAPROXEN:Immunosuppressant drug therapy:1.81), (NEBIVOLOL HYDROCHLORIDE:Immunosuppressant drug therapy:0.48), (NORTRIPTYLINE:Immunosuppressant drug therapy:0.17), (OXYBUTYNIN CHLORIDE:Immunosuppressant drug therapy:0.3), (PANTOPRAZOLE MAGNESIUM:Immunosuppressant drug therapy:1.06), (PAZOPANIB HYDROCHLORIDE:Immunosuppressant drug therapy:1.05), (PENTOSTATIN:Immunosuppressant drug therapy:0.31), (PHENTOLAMINE:Immunosuppressant drug therapy:0.05), (PIROXICAM:Immunosuppressant drug therapy:0.05), (PITAVASTATIN CALCIUM:Immunosuppressant drug therapy:0.42), (POLYMYXIN:Immunosuppressant drug therapy:0.05), (PONATINIB:Immunosuppressant drug therapy:1.15), (PROCHLORPERAZINE MALEATE:Immunosuppressant drug therapy:0.54), (RALOXIFENE HYDROCHLORIDE:Immunosuppressant drug therapy:0.12), (RANOLAZINE:Immunosuppressant drug therapy:0.86), (REGORAFENIB:Immunosuppressant drug therapy:0.14), (RETINOL:Immunosuppressant drug therapy:0.54), (RIZATRIPTAN BENZOATE:Immunosuppressant drug therapy:0.02), (ROFLUMILAST:Immunosuppressant drug therapy:0.63), (SENNOSIDES:Immunosuppressant drug therapy:0.52), (SEVELAMER:Immunosuppressant drug therapy:0.12), (SITAGLIPTIN:Immunosuppressant drug therapy:0.57), (SODIUM CITRATE:Immunosuppressant drug therapy:0.25), (SUCCINYLCHOLINE CHLORIDE:Immunosuppressant drug therapy:0.14), (SUFENTANIL:Immunosuppressant drug therapy:0.18), (SUMATRIPTANSUMATRIPTAN SUCCINATE:Immunosuppressant drug therapy:0.28), (THIOGUANINE:Immunosuppressant drug therapy:0.08), (TIMOLOL:Immunosuppressant drug therapy:0.29), (TINZAPARIN SODIUM:Immunosuppressant drug therapy:0.45), (TIZANIDINE:Immunosuppressant drug therapy:0.29), (UMECLIDINIUM BROMIDEVILANTEROL TRIFENATATE:Immunosuppressant drug therapy:0.69), (VARENICLINE TARTRATE:Immunosuppressant drug therapy:0.68), (VECURONIUM BROMIDE:Immunosuppressant drug therapy:0.45), (WARFARIN POTASSIUM:Immunosuppressant drug therapy:0.17), (.ALPHA.1-PROTEINASE INHIBITOR HUMAN:Immunosuppression:0.05), (ABACAVIR SULFATE:Immunosuppression:0.02), (ABACAVIR SULFATEDOLUTEGRAVIR SODIUMLAMIVUDINE:Immunosuppression:0.67), (ACETAMINOPHENBUTALBITALCA FFEINE:Immunosuppression:0.93), (ACETAMINOPHENTRAMADOL:Immunosuppression:0.43), (ACLARUBICIN HYDROCHLORIDE:Immunosuppression:0.08), (ADO-TRASTUZUMAB EMTANSINE:Immunosuppression:0.2), (ALCOHOL:Immunosuppression:0.2), (AMILORIDE HYDROCHLORIDEHYDROCHLOROTHIAZIDE:Immunosuppression:0.47), (AMINO ACIDSDEXTROSEELECTROLYTES NOS:Immunosuppression:0.28), (AMINOSALICYLIC ACID:Immunosuppression:0.14), (AMISULPRIDE:Immunosuppression:0.47), (AMLODIPINE BESYLATEVALSARTAN:Immunosuppression:0.02), (AMLODIPINEVALSARTAN:Immun osuppression:0.34), (APREPITANT:Immunosuppression:0.33),(ARMODAFINIL:Immunosuppre ssion:0.58), (ASUNAPREVIR:Immunosuppression:0.78), (ATRACURIUM:Immunosuppression: 0.52), (ATROPINE:Immunosuppression:0.25), (AXITINIB:Immunosuppression:0.27), (AZELAS TINE:Immunosuppression:0.08), (AZILSARTAN KAMEDOXOMIL:Immunosuppression:0.46), (AZOSEMIDE:Immunosuppression:0.36), (BENA ZEPRILBENAZEPRIL HYDROCHLORIDE:Immunosuppression:0.59), (BETAMETHASONE DIPROPIONATE:Immunosuppression:0.29), (BETAMIPRONPANIPENEM:Immunosuppressi on:0.06), (BICARBONATE ION:Immunosuppression:0.63), (BILASTINE:Immunosuppression:0.21), (BUDESONIDEFOR MOTEROL FUMARATE:Immunosuppression:0.33), (BUPRENORPHINE HYDROCHLORIDE:Immunosuppression:0.71), (BUPRENORPHINE HYDROCHLORIDENALOXONE HYDROCHLORIDE:Immunosuppression:0.29), (CANAGLIFLOZINMETFORMIN HYDROCHLORIDE:Immunosuppression:0.46), (CANDESARTAN CILEXETILHYDROCHLOROTHIAZIDE:Immunosuppression:1.81), (CAPSAICIN:Immunosu ppression:0.11), (CARBIMAZOLE:Immunosuppression:0.5), (CARMUSTINE:Immunosuppressi on:0.85), (CARVEDILOL PHOSPHATE:Immunosuppression:0.71), (CASANTHRANOLDOCUSATE SODIUM:Immunosuppression:0.07), (CORTICOTROPIN:Immunosuppression:0.01), (CYAME MAZINE:Immunosuppression:0.29), (CYCLOPHOSPHAMIDECYTARABINEDEXAMETH ASONEDOXORUBICINMETHOTREXATEVINCRISTINE:Immunosuppression:0.03), (CYP ROHEPTADINE:Immunosuppression:0.47), (DESFLURANE:Immunosuppression:0.62), (DESV ENLAFAXINE SUCCINATE:Immunosuppression:0.36), (DEVICE:Immunosuppression:0.01), (DEXAMETHAS ONE ACETATE:Immunosuppression:1.03), (DIMETHICONE:Immunosuppression:0.47), (DIPYRIDA MOLE:Immunosuppression:0.47), (DOBUTAMINE HYDROCHLORIDE:Immunosuppression:0.42), (DOBUTAMINEDOBUTAMINE HYDROCHLORIDE:Immunosuppression:0.94), (DOFETILIDE:Immunosuppression:0.27), (DO XYCYCLINE HYCLATE:Immunosuppression:0.02), (DULOXETINE:Immunosuppression:0.55), (ECONAZO LE:Immunosuppression:1.32), (EDOXABAN TOSYLATE:Immunosuppression:1.35), (ELBASVIRGRAZOPREVIR:Immunosuppression:0.42), (ELETRIPTAN HYDROBROMIDE:Immunosuppression:0.91), (ELOTUZUMAB:Immunosuppression:1.1), (ELTROMBOPAG OLAMINE:Immunosuppression:0.07), (EMPAGLIFLOZINLINAGLIPTIN:Immunosuppression:0.18), (EPIRUBICIN:Immunosuppression:0.36), (EPLERENONE:Immunosuppression:0.94), (ESTROGENS, CONJUGATEDMEDROXYPROGESTERONE ACETATE:Immunosuppression:0.44), (ETIMICIN SULFATE:Immunosuppression:0.54), (ETOPOSIDE PHOSPHATE:Immunosuppression:0.67), (ETORICOXIB:Immunosuppression:0.96), (EXEMES TANE:Immunosuppression:0.1), (FERROUS SULFATE ANHYDROUS:Immunosuppression:0.15), (FERROUS SULFATEGLYCINE:Immunosuppression:0.63), (FESOTERODINE FUMARATE:Immunosuppression:0.7), (FLUCYTOSINE:Immunosuppression:0.06), (FLUOCIN ONIDE:Immunosuppression:0.12), (FLUTICASONE:Immunosuppression:0.92), (FLUTICASON E FUROATE:Immunosuppression:0.07), (FLUVOXAMINE MALEATE:Immunosuppression:0.18), (FONDAPARINUX SODIUM:Immunosuppression:0.53), (FORMOTEROL:Immunosuppression:0.49), (FORMOTER OL FUMARATE:Immunosuppression:0.28), (FORMOTEROL FUMARATE DIHYDRATEMOMETASONE FUROATE:Immunosuppression:0.27), (FOSPHENYTOINFOSPHENYTOIN SODIUM:Immunosuppression:0.4), (GLUCOSAMINE:Immunosuppression:0.1), (GLYCOPYRR ONIUMINDACATEROL:Immunosuppression:0.71), (HERBALSSAW PALMETTO:Immunosuppression:0.25), (HYDROCHLOROTHIAZIDELISINOPRIL:Immunos uppression:0.99), (HYDROXYCHLOROQUINE DIPHOSPHATE:Immunosuppression:0.26), (HYDROXYZINE PAMOATE:Immunosuppression:1.24), (IBRITUMOMAB TIUXETAN:Immunosuppression:1.06), (IDARUBICIN HYDROCHLORIDE:Immunosuppression:0.6), (IDARUCIZUMAB:Immunosuppression:0.11), (I DELALISIB:Immunosuppression:0.83), (ILOPROST:Immunosuppression:0.8), (IMIPRAMINE HYDROCHLORIDE:Immunosuppression:0.3), (INTERFERON GAMMA-1B:Immunosuppression:0.3), (IRINOTECAN HYDROCHLORIDE:Immunosuppression:0.22), (ISOSORBIDE:Immunosuppression:0.81), (KE TAMINE HYDROCHLORIDE:Immunosuppression:0.25), (LEVOLEUCOVORIN CALCIUM:Immunosuppression:0.24), (LEVOTHYROXINE SODIUMPOTASSIUM IODIDE:Immunosuppression:0.42), (LISDEXAMFETAMINE DIMESYLATE:Immunosuppression:0.16), (LOMITAPIDE MESYLATE:Immunosuppression:0.01), (LOMUSTINE:Immunosuppression:0.02), (METHENA MINE HIPPURATE:Immunosuppression:0.63), (METHIMAZOLE:Immunosuppression:1.3), (METILD IGOXIN:Immunosuppression:0.44), (MORPHINE SULFATENALTREXONE HYDROCHLORIDE:Immunosuppression:0.13), (NABUMETONE:Immunosuppression:0.09), ( NALOXONE:Immunosuppression:0.54), (NALOXONE HYDROCHLORIDETILIDINE HYDROCHLORIDE:Immunosuppression:0.77), (NAPROXEN SODIUM:Immunosuppression:0.65), (NIRAPARIB:Immunosuppression:0.1), (OXACILLIN SODIUM:Immunosuppression:0.13), (PANTOPRAZOLE MAGNESIUM:Immunosuppression:0.68), (PAROXETINEPAROXETINE HYDROCHLORIDE:Immunosuppression:1.4), (PASIREOTIDE:Immunosuppression:0.18), (PE GASPARGASE:Immunosuppression:0.87), (PEGINTERFERON BETA-1A:Immunosuppression:0.19), (PEMETREXED DISODIUM:Immunosuppression:0.02), (PIPERACILLIN:Immunosuppression:0.56), (PIROXICAM: Immunosuppression:0.28), (PITAVASTATIN CALCIUM:Immunosuppression:0.74), (PLANTAGO SEED:Immunosuppression:0.2), (POLYMYXIN B SULFATE:Immunosuppression:0.09), (PRAMIPEXOLE DIHYDROCHLORIDE:Immunosuppression:0.61), (PRAZEPAM:Immunosuppression:0.3), (PRI STINAMYCIN:Immunosuppression:0.4), (RALOXIFENE HYDROCHLORIDE:Immunosuppression:0.32), (RALTEGRAVIR POTASSIUM:Immunosuppression:1.24), (RANOLAZINE:Immunosuppression:1.1), (RIVASTIG MINE:Immunosuppression:0.25), (ROFLUMILAST:Immunosuppression:0.6), (ROPIVACAINE: Immunosuppression:0.15), (SAQUINAVIR:Immunosuppression:0.28), (SCOPOLAMINE HYDROBROMIDE:Immunosuppression:0.24), (SECUKINUMAB:Immunosuppression:1.33), (S ILVER SULFADIAZINE:Immunosuppression:0.04), (SODIUM CITRATE:Immunosuppression:0.35), (SODIUM NITROPRUSSIDE:Immunosuppression:0.01), (SODIUM OXYBATE:Immunosuppression:0.47), (SODIUM PHOSPHATE, DIBASICSODIUM PHOSPHATE, MONOBASIC:Immunosuppression:1.3), (SUCCINYLCHOLINE CHLORIDE:Immunosuppression:0.54), (SUGAMMADEX:Immunosuppression:0.21), (SUMAT RIPTAN SUCCINATE:Immunosuppression:0.19), (SUMATRIPTANSUMATRIPTAN SUCCINATE:Immunosuppression:0.89), (TERIFLUNOMIDE:Immunosuppression:0.5), (TETR ACYCLINE:Immunosuppression:0.42), (THIAMINE:Immunosuppression:0.24), (THROMBOPO IETIN:Immunosuppression:0.4), (TIMOLOL:Immunosuppression:0.24), (TIOTROPIUM:Immun osuppression:0.79), (TIZANIDINE HYDROCHLORIDE:Immunosuppression:0.69), (TOLTERODINE TARTRATE:Immunosuppression:0.57), (TRAMETINIB:Immunosuppression:0.32), (TRIAMCI NOLONE ACETONIDE:Immunosuppression:0.25), (TRICHLORMETHIAZIDE:Immunosuppression:0.09), (UMECLIDINIUM BROMIDEVILANTEROL TRIFENATATE:Immunosuppression:0.2), (VARENICLINE TARTRATE:Immunosuppression:1.11), (VENETOCLAX:Immunosuppression:0.07), (VITAMIN A:Immunosuppression:0.59), (WARFARIN POTASSIUM:Immunosuppression:0.08), (ZINC:Immunosuppression:0.61), (ZONISAMIDE:Im munosuppression:0.14), (.ALPHA.1-PROTEINASE INHIBITOR HUMAN:Impaired gastric emptying:0.04), (ACARBOSE:Impaired gastric emptying:0.02), (ACETAMINOPHENCODEINE PHOSPHATE:Impaired gastric emptying:1.14), (ACETAZOLAMIDE:Impaired gastric emptying:0.14), (ALCOHOL:Impaired gastric emptying:0.17), (ANASTROZOLE:Impaired gastric emptying:0.22), (CALCIUM ACETATE:Impaired gastric emptying:0.75), (CALCIUM CARBONATECHOLECALCIFEROL:Impaired gastric emptying:0.81), (CALCIUM CITRATE:Impaired gastric emptying:0.35), (CANAGLIFLOZINMETFORMIN HYDROCHLORIDE:Impaired gastric emptying:0.11), (CANDESARTAN:Impaired gastric emptying:0.79), (CANDESARTAN CILEXETIL:Impaired gastric emptying:0.62), (CANDESARTAN CILEXETILHYDROCHLOROTHIAZIDE:Impaired gastric emptying:0.18), (CLINDAMYCINCLINDAMYCIN PHOSPHATE:Impaired gastric emptying:0.95), (CLOZAPINE:Impaired gastric emptying:0.28), (DACLIZUMAB:Impaired gastric emptying:0.56), (DASATINIB:Impaired gastric emptying:0.18), (DICLOFENAC:Impaired gastric emptying:0.6), (DOFETILIDE:Impaired gastric emptying:0.06), (DOXAZOSIN MESYLATE:Impaired gastric emptying:0), (DOXAZOSINDOXAZOSIN MESYLATE:Impaired gastric emptying:0.2), (EMPAGLIFLOZINLINAGLIPTIN:Impaired gastric emptying:0.09), (ENALAPRIL:Impaired gastric emptying:1.56), (ESTROGENS, CONJUGATEDMEDROXYPROGESTERONE ACETATE:Impaired gastric emptying:0.15), (EVEROLIMUS:Impaired gastric emptying:0.22), (EVOLOCUMAB:Impaired gastric emptying:1.17), (EZETIMIBESIMVASTATIN:Impaired gastric emptying:0.09), (FLUTICASONE FUROATE:Impaired gastric emptying:0.05), (FORMOTEROL FUMARATE DIHYDRATEMOMETASONE FUROATE:Impaired gastric emptying:0.23), (GLICLAZIDE:Impaired gastric emptying:0.07), (IBRUTINIB:Impaired gastric emptying:0.52), (ILOPROST:Impaired gastric emptying:0.07), (IMIPRAMINE HYDROCHLORIDE:Impaired gastric emptying:0.43), (INDOMETHACIN:Impaired gastric emptying:0.2), (INFLUENZA VIRUS VACCINE:Impaired gastric emptying:0.1), (IPRATROPIUM:Impaired gastric emptying:0.37), (ISOSORBIDE MONONITRATE:Impaired gastric emptying:0.96), (LABETALOLLABETALOL HYDROCHLORIDE:Impaired gastric emptying:0.57), (LENALIDOMIDE:Impaired gastric emptying:0.56), (LEVALBUTEROL HYDROCHLORIDE:Impaired gastric emptying:0.52), (LIOTHYRONINE SODIUM:Impaired gastric emptying:0.09), (LITHIUM CARBONATE:Impaired gastric emptying:0.64), (MEDROXYPROGESTERONE ACETATE:Impaired gastric emptying:0.26), (METHENAMINE HIPPURATE:Impaired gastric emptying:0.03), (METHYLPREDNISOLONE ACETATE:Impaired gastric emptying:0.33), (NADOLOL:Impaired gastric emptying:0.19), (NILOTINIB:Impaired gastric emptying:0.34), (PAROXETINEPAROXETINE HYDROCHLORIDE:Impaired gastric emptying:0.8), (PEGINTERFERON BETA-1A:Impaired gastric emptying:0.05), (PRAMIPEXOLE:Impaired gastric emptying:0.33), (PRIMIDONE:Impaired gastric emptying:0.96), (ROFLUMILAST:Impaired gastric emptying:0.28), (SACUBITRILVALSARTAN:Impaired gastric emptying:0.16), (SECUKINUMAB:Impaired gastric emptying:0.49), (SUMATRIPTAN:Impaired gastric emptying:0.56), (TAMSULOSIN:Impaired gastric emptying:1.36), (TERIFLUNOMIDE:Impaired gastric emptying:0.91), (THYROID, PORCINE:Impaired gastric emptying:0.21), (TIMOLOL:Impaired gastric emptying:0.28), (VILAZODONE HYDROCHLORIDE:Impaired gastric emptying:0.28), (VITAMIN B:Impaired gastric emptying:0.76), (ZONISAMIDE:Impaired gastric emptying:0.24), (ACARBOSE:Incontinence:0.19), (ACETAMINOPHENOXYCODONEOXYC ODONE HYDROCHLORIDE:Incontinence:0.29), (ACETAZOLAMIDE:Incontinence:0.28), (AGALSID ASE BETA:Incontinence:0.04), (AMISULPRIDE:Incontinence:0.09), (AMLODIPINE BESYLATEHYDROCHLOROTHIAZIDEOLMESARTAN MEDOXOMIL:Incontinence:0.17), (AZITHROMYCIN ANHYDROUS:Incontinence:0.07), (BENAZEPRILBENAZEPRIL HYDROCHLORIDE:Incontinence:0.1), (BRINZOLAMIDE:Incontinence:0.21), (BROMAZEPA M:Incontinence:0.42), (BUDESONIDEFORMOTEROL FUMARATE:Incontinence:0.14), (BUMETANIDE:Incontinence:0.81), (CALCIUM ACETATE:Incontinence:0.31), (CALCIUMMAGNESIUMZINC:Incontinence:0.06), (CANAG LIFLOZINMETFORMIN HYDROCHLORIDE:Incontinence:0.23), (CANDESARTAN CILEXETIL:Incontinence:0.96), (CANDESARTAN CILEXETILHYDROCHLOROTHIAZIDE:Incontinence:0.82), (CAPECITABINE:Incontinence: 0.05), (CAPTOPRIL:Incontinence:0.46), (CARFILZOMIB:Incontinence:0.22), (CARVEDILOL PHOSPHATE:Incontinence:0.93), (CIMETIDINE:Incontinence:0.5), (CLINDAMYCINCLIND AMYCIN PHOSPHATE:Incontinence:0.73), (CLONIDINE HYDROCHLORIDE:Incontinence:0.06), (CLOTRIMAZOLE:Incontinence:0.42), (COD LIVER OIL:Incontinence:0.12), (COLCHICINE:Incontinence:0.63), (CORTICOTROPIN:Incontinence:0.09), (DABIGATRAN:Incontinence:0.03), (DACLIZUMAB:Incontinence:0.31), (DASATINIB:Inc ontinence:0.62), (DIMENHYDRINATE:Incontinence:0.37), (DOXEPIN:Incontinence:0.11), (DO XEPIN HYDROCHLORIDE:Incontinence:0.18), (DRONABINOL:Incontinence:0.11), (ELETRIPTAN HYDROBROMIDE:Incontinence:0.83), (EMPAGLIFLOZINLINAGLIPTIN:Incontinence:0.47), (EPLERENONE:Incontinence:0.19), (ESOMEPRAZOLE:Incontinence:0.59), (ESTROGENS, CONJUGATEDMEDROXYPROGESTERONE ACETATE:Incontinence:0.84), (ESZOPICLONE:Incontinence:0.88), (ETORICOXIB:Incontinen ce:0.22), (EVEROLIMUS:Incontinence:0.34), (EVOLOCUMAB:Incontinence:1.49), (HEPARIN SODIUM:Incontinence:0.46), (HYDROCHLOROTHIAZIDEIRBESARTAN:Incontinence:0.45), (IMIPRAMINE HYDROCHLORIDE:Incontinence:0.46), (ISOSORBIDE:Incontinence:0.74), (LABETALOLLA BETALOL HYDROCHLORIDE:Incontinence:0.82), (LEDIPASVIRSOFOSBUVIR:Incontinence:0.01), (LE RCANIDIPINE:Incontinence:1.01), (LERCANIDIPINE HYDROCHLORIDE:Incontinence:0.39), (LEVALBUTEROL HYDROCHLORIDE:Incontinence:0.73), (LIDOCAINE HYDROCHLORIDE:Incontinence:0.42), (LISDEXAMFETAMINE DIMESYLATE:Incontinence:0.78), (LOMITAPIDE MESYLATE:Incontinence:0.23), (METAXALONE:Incontinence:1.47), (METHIMAZOLE:Incon tinence:0.09), (METHYLPREDNISOLONE SODIUM SUCCINATE:Incontinence:0.95), (METRONIDAZOLEMETRONIDAZOLE HYDROCHLORIDE:Incontinence:0.35), (MOXIFLOXACIN HYDROCHLORIDE:Incontinence:0.85), (NALTREXONE:Incontinence:0.16), (NILOTINIB:Inc ontinence:0.8), (NORTRIPTYLINE HYDROCHLORIDE:Incontinence:0.06), (PEGINTERFERON BETA-1A:Incontinence:0.41), (PHENOBARBITAL:Incontinence:0.51), (PONATINIB:Incontinence:0.15), (RANOLAZINE:Incontinence:1.45), (ROFLUMILAST:Incontinence:0.49), (SACUBITRILV ALSARTAN:Incontinence:1.07), (SELEGILINE:Incontinence:0.1), (SODIUM BICARBONATE:Incontinence:0.06), (TERBINAFINE HYDROCHLORIDE:Incontinence:0.03), (TERBUTALINE SULFATE:Incontinence:0.04), (TETRACYCLINE:Incontinence:0.47), (UMECLIDINIUM BROMIDEVILANTEROL TRIFENATATE:Incontinence:0.62), (VILAZODONE HYDROCHLORIDE:Incontinence:0.56), (AMIODARONE:Increased appetite:0.11), (ANASTROZOLE:Increased appetite:0.45), (BUMETANIDE:Increased appetite:0.58), (BUPROPION HYDROCHLORIDE:Increased appetite:0.65), (CALCIUM CARBONATECHOLECALCIFEROL:Increased appetite:0.43), (CLINDAMYCINCLINDAMYCIN PHOSPHATE:Increased appetite:0.38), (COLCHICINE:Increased appetite:0.03), (DASATINIB:Increased appetite:0.16), (EPINEPHRINE:Increased appetite:0.25), (GLIPIZIDE:Increased appetite:0.06), (IDELALISIB:Increased appetite:0.06), (ILOPROST:Increased appetite:0.17), (LOVASTATIN:Increased appetite:0.06), (POLYETHYLENE GLYCOLS:Increased appetite:0.27), (PRAVASTATIN SODIUM:Increased appetite:0.23), (RAMIPRIL:Increased appetite:0.57), (SACUBITRILVALSARTAN:Increased appetite:0.35), (TERIFLUNOMIDE:Increased appetite:0.12), (THALIDOMIDE:Increased appetite:0.31), (TRIAMCINOLONE ACETONIDE:Increased appetite:0.05), (UMECLIDINIUM BROMIDEVILANTEROL TRIFENATATE:Increased appetite:0.14), (WARFARIN:Increased appetite:0.87), (ESMOLOL HYDROCHLORIDE:Induction and maintenance of anaesthesia:0.6), (ALCOHOL:Induction of anaesthesia:0.05), (AMPICILLIN SODIUMSULBACTAM SODIUM:Induction of anaesthesia:0.08), (BUMETANIDE:Induction of anaesthesia:0.19), (CALCITRIOL:Induction of anaesthesia:0.39), (CALCIUM CARBONATECHOLECALCIFEROL:Induction of anaesthesia:0.32), (CARBON DIOXIDE:Induction of anaesthesia:0.25), (CEPHALEXIN:Induction of anaesthesia:0.35), (CLOZAPINE:Induction of anaesthesia:0.33), (DEXLANSOPRAZOLE:Induction of anaesthesia:0.17), (DOCUSATE:Induction of anaesthesia:0.13), (ERYTHROPOIETIN:Induction of anaesthesia:0.01), (EVEROLIMUS:Induction of anaesthesia:0.27), (FLECAINIDE:Induction of anaesthesia:0.43), (FLUCONAZOLE:Induction of anaesthesia:0.03), (FLUTICASONE PROPIONATE:Induction of anaesthesia:0.53), (FOSPHENYTOIN SODIUM:Induction of anaesthesia:0.49), (FOSPHENYTOINFOSPHENYTOIN SODIUM:Induction of anaesthesia:0.92), (GLIPIZIDE:Induction of anaesthesia:0.09), (HEPARIN CALCIUM:Induction of anaesthesia:0.04), (LACOSAMIDE:Induction of anaesthesia:0.04), (LERCANIDIPINE:Induction of anaesthesia:0.7), (METARAMINOL:Induction of anaesthesia:0.32), (MOXIFLOXACIN HYDROCHLORIDE:Induction of anaesthesia:0.19), (NADROPARIN CALCIUM:Induction of anaesthesia:0.1), (NALOXONE:Induction of anaesthesia:0.42), (NALOXONE HYDROCHLORIDEOXYCODONE HYDROCHLORIDE:Induction of anaesthesia:0.07), (NYSTATIN:Induction of anaesthesia:0.07), (OMEPRAZOLE MAGNESIUM:Induction of anaesthesia:0.53), (OSELTAMIVIR:Induction of anaesthesia:0.01), (OXCARBAZEPINE:Induction of anaesthesia:1.02), (PRAVASTATIN:Induction of anaesthesia:0.23), (PRIMIDONE:Induction of anaesthesia:0.11), (RANOLAZINE:Induction of anaesthesia:0.09), (SACUBITRILVALSARTAN:Induction of anaesthesia:0.21), (TOPIRAMATE:Induction of anaesthesia:0.9), (VALGANCICLOVIR HYDROCHLORIDE:Induction of anaesthesia:0.05), (WARFARIN SODIUM:Induction of anaesthesia:0.35), (BUMETANIDE:Infarction:0.03), (FOLIC ACID:Infarction:0.28), (IBRUTINIB:Infarction:0.04), (INSULIN LISPRO:Infarction:0.05), (LERCANIDIPINE:Infarction:0.15), (WARFARIN:Infarction:0.55), (A BOBOTULINUMTOXINA:Infection:0.65), (ACETAMINOPHENDIPHENHYDRAMINE HYDROCHLORIDE:Infection:0.21), (ACETAMINOPHENOXYCODONEOXYCODONE HYDROCHLORIDE:Infection:1.97), (AGALSIDASE BETA:Infection:1.62), (ALBIGLUTIDE:Infection:0.13), (ALBUTEROLGUAIFENESIN:Infecti on:0.27), (ALBUTEROLIPRATROPIUM BROMIDE:Infection:0.21), (ALGLUCOSIDASE ALFA:Infection:1.05), (ALTHIAZIDESPIRONOLACTONE:Infection:0.19), (AMINO ACIDSDEXTROSEELECTROLYTES NOS:Infection:1.03), (AMINOSALICYLIC ACID:Infection:0.87), (AMLODIPINE BESYLATETELMISARTAN:Infection:0.56), (AMMONIUM CHLORIDEDEXTROMETHORPHANSODIUM CITRATE:Infection:0.48), (AMOXAPINE:Infection:0.29), (AMOXICILLINCLARITHROMYC INESOMEPRAZOLE:Infection:0.64), (AMPHOTERICIN BDIMYRISTOYLPHOSPHATIDYLCHOLINE, DL-DIMYRISTOYLPHOSPHATIDYLGLYCEROL, DL-:Infection:1), (ANAGRELIDE:Infection:0.34), (ANAGRELIDE HYDROCHLORIDE:Infection:0.3), (ANTIHISTAMINES NOS:Infection:0.92), (ARSENIC TRIOXIDE:Infection:0.5), (ARTESUNATE:Infection:0.04), (ASCORBIC ACIDBIOTINCYANOCOBALAMINFOLIC ACIDNIACINPANTOTHENIC ACIDPYRIDOXINERIBOFLAVINTHIAMINE:Infection:0.23), (ASPIRINDIHYDROXYAL UMINUM AMINOACETATE ANHYDROUSMAGNESIUM CARBONATE:Infection:0.24), (ASUNAPREVIR:Infection:0.34), (ATAZANAVIR:Infection:0.49), (ATAZANAVIRRITONAVIR:Infection:0.13), (ATENOLOLCHLORTHALIDONE:Infectio n:0.42), (AVELUMAB:Infection:0.04), (BALSALAZIDE DISODIUM:Infection:0.54), (BECLOMETHASONE:Infection:1.54), (BERAPROST:Infection:0.42), (BETAMETHASONE DIPROPIONATECALCIPOTRIENECALCIPOTRIENE HYDRATE:Infection:0.53), (BETAMETHASONE VALERATEFUSIDIC ACID:Infection:0.31), (BETHANECHOL CHLORIDE:Infection:0.39), (BLESELUMAB:Infection:1.7), (BOSUTINIB:Infection:0.29), (BO TULINUM TOXIN TYPE A:Infection:0.72), (BOTULISM ANTITOXIN:Infection:0.37), (BUPIVACAINE HYDROCHLORIDE:Infection:0.73), (BUPRENORPHINE HYDROCHLORIDENALOXONE HYDROCHLORIDE:Infection:1.47), (CABAZITAXEL:Infection:0.78), (CABOZANTINIB:Infe ction:0.17), (CALCITONIN SALMON:Infection:1.63), (CALCIUM CARBONATECHOLECALCIFEROLMAGNESIUM CARBONATE:Infection:0.51), (CALCIUM CARBONATEERGOCALCIFEROL:Infection:0.22), (CALCIUM CARBONATEERGOCALCIFEROLRETINOL:Infection:1.53), (CALCIUM PANTOTHENATECYANOCOBALAMINNIACINAMIDEPYRIDOXINE HYDROCHLORIDERIBOFLAVINTHIAMINE MONONITRATE:Infection:0.12), (CALCIUM POLYCARBOPHIL:Infection:0.82), (CALCIUMMAGNESIUM:Infection:1.13), (CAMPHOR (NATURAL)MENTHOLPRAMOXINE HYDROCHLORIDE:Infection:0.18), (CARBIDOPA:Infection:0.39), (CARBIMAZOLE:Infectio n:1.56), (CARTEOLOL:Infection:0.17), (CARVEDILOL PHOSPHATE:Infection:2.11), (CASANTHRANOLDOCUSATE SODIUM:Infection:1.11), (CEFMETAZOLE:Infection:0.1), (CEFPIROME SULFATE:Infection:1.2), (CEFPROZIL:Infection:1.1), (CELIPROLOL:Infection:0.38), (CEVIM ELINE HYDROCHLORIDE:Infection:0.11), (CHLORDIAZEPOXIDE HYDROCHLORIDECLIDINIUM BROMIDE:Infection:0.61), (CINACALCET:Infection:0.24), (CLOMIPRAMINE HYDROCHLORIDE:Infection:1.84), (COBICISTATDARUNAVIR ETHANOLATE:Infection:0.16), (COBICISTATELVITEGRAVIREMTRICITABINETENOF OVIR ALAFENAMIDE FUMARATE:Infection:0.69), (CODEINE PHOSPHATEGUAIFENESIN:Infection:0.66), (CORTISONE ACETATE:Infection:0.23), (CROTALIDAE POLYVALENT IMMUNE FAB (OVINE):Infection:0.11), (CYAMEMAZINE:Infection:1.38), (CYCLOPHOSPHAMIDECYTA RABINEDEXAMETHASONEDOXORUBICINMETHOTREXATEVINCRISTINE:Infectio n:0.5), (CYPROHEPTADINE HYDROCHLORIDE:Infection:1.15), (DACLATASVIR:Infection:0.44), (DALFOPRISTINQUI NUPRISTIN:Infection:0.06), (DAPAGLIFLOZIN PROPANEDIOLMETFORMIN HYDROCHLORIDE:Infection:0.2), (DARUNAVIRRITONAVIR:Infection:0.11), (DASABUVI ROMBITASVIRPARITAPREVIRRITONAVIR:Infection:0.6), (DEGARELIX:Infection:0.29), (DEOXYRIBONUCLIEC ACID:Infection:0.56), (DESFLURANE:Infection:0.91), (DESLANOSIDE:Infection:0.25), (DESO GESTRELETHINYL ESTRADIOL:Infection:0.1), (DISOPYRAMIDE:Infection:0.1), (DOCUSATE SODIUMSENNOSIDE ASENNOSIDE B:Infection:0.13), (EDOXABAN TOSILATE MONOHYDRATE:Infection:0.19), (ELBASVIRGRAZOPREVIR:Infection:0.54), (ELETRIPT AN HYDROBROMIDE:Infection:2.29), (ELTROMBOPAG OLAMINE:Infection:0.81), (EMTRICITABINERILPIVIRINETENOFOVIR DISOPROXIL FUMARATE:Infection:0.65), (ENALAPRIL MALEATELERCANIDIPINE HYDROCHLORIDE:Infection:0.1), (EPACADOSTAT:Infection:0.02), (ERIBULIN:Infection:0.63), (ESMOLOL HYDROCHLORIDE:Infection:1.62), (ESTRIOL:Infection:0.27), (ETHINYL ESTRADIOLETHYNODIOL:Infection:0.19), (ETHINYL ESTRADIOLNORGESTREL:Infection:0.54), (ETOPOSIDE PHOSPHATE:Infection:1.55), (ETRETINATE:Infection:1.15), (FELBAMATE:Infection:0.58), (F ERROUS SULFATEGLYCINE:Infection:1.19), (FLUOROURACILLEUCOVORIN CALCIUMOXALIPLATIN:Infection:0.14), (FLUPHENAZINE:Infection:0.02), (FLUTAMIDE: Infection:0.11), (FLUTICASONE PROPIONATEFORMOTEROL FUMARATE:Infection:0.37), (FLUVOXAMINE MALEATE:Infection:1.4), (FORMOTEROL FUMARATEMOMETASONE FUROATE:Infection:1.59), (GABEXATE MESYLATE:Infection:0.6), (GARENOXACIN:Infection:1), (GLIPIZIDEMETFORMIN HYDROCHLORIDE:Infection:0.26), (GLYCOPYRROLATEINDACATEROL MALEATE:Infection:0.76), (GUANFACINE:Infection:0.86), (GUANFACINE HYDROCHLORIDE:Infection:1.07), (HUMAN BLOOD CELLS:Infection:0.23), (HYDROCORTISONE SODIUM PHOSPHATE:Infection:0.03), (HYDROXYCHLOROQUINE DIPHOSPHATE:Infection:1.07), (HYOSCYAMINE:Infection:1.18), (IBRITUMOMAB TIUXETAN:Infection:1.83), (IDARUCIZUMAB:Infection:0.29), (IMIPRAMINE HYDROCHLORIDE:Infection:1.64), (IMIQUIMOD:Infection:0.46), (INFLIXIMAB-DYYB:Infection:0.93), (INTERFERON:Infection:1.92), (INTERFERON ALFA-2A:Infection:2.05), (INTERFERON ALFA-2A OR INTERFERON ALFA-2B:Infection:0.32), (INTERFERON GAMMA:Infection:0.01), (INTERFERON GAMMA-1B:Infection:1.7), (IOPROMIDE:Infection:0.08), (IRONPOLYSACCHARIDES:Infection:0.45), (ISOFLURANE:Infection:0.9), (LACTITOL:Infection:0.05), (LANREOTIDE:Infection:0.04), (L ANREOTIDE ACETATE:Infection:0.66), (LEVAMLODIPINE BESYLATE:Infection:0.11), (LEVOCARNITINE HYDROCHLORIDE:Infection:0.48), (LEVOMEFOLATE CALCIUM:Infection:0.13), (LEVOMILNACIPRAN HYDROCHLORIDE:Infection:0), (LEVOSIMENDAN:Infection:0.03), (LEVOTHYROXINE SODIUMPOTASSIUM IODIDE:Infection:0.63), (LINSIDOMINE:Infection:0.12), (LOMITAPIDE MESYLATE:Infection:0.6), (LOMUSTINE:Infection:0.8), (LUMACAFTOR:Infection:0.69), (M AGNESIUM ASPARTATEPOTASSIUM ASPARTATE:Infection:0.78), (MAGNESIUM GLUCONATE:Infection:0.48), (MAGNESIUM PIDOLATE:Infection:0.13), (MEPOLIZUMAB:Infection:1.23), (METFORMIN HYDROCHLORIDEVILDAGLIPTIN:Infection:1), (METHENOLONE ACETATE:Infection:0.02), (METHOTREXATEMETHOTREXATE SODIUM:Infection:0.09), (METHYLENE BLUE:Infection:0.37), (METHYLPREDNISOLONE OR METHYLPREDNISOLONE ACETATE:Infection:0.03), (MIGLITOL:Infection:0.22), (MILRINONE LACTATE:Infection:0.01), (MINODRONIC ACID:Infection:0.43), (MOGAMULIZUMAB:Infection:0.86), (MORPHINEMORPHINE HYDROCHLORIDEMORPHINE SULFATEMORPHINE TARTRATE:Infection:0.03), (NARATRIPTAN HYDROCHLORIDE:Infection:0.16), (NUSINERSEN:Infection:0.31), (OLOPATADINE:Infectio n:1.85), (OXYMORPHONE:Infection:0.09), (PANTOPRAZOLE MAGNESIUM:Infection:2.58), (PARATHYROID HORMONE:Infection:0.01), (PEGINTERFERON BETA-1A:Infection:1.2), (PEGVISOMANT:Infection:0.16), (PENTOBARBITAL:Infection:0.18), (PEN TOSTATIN:Infection:1.95), (PERPHENAZINE:Infection:0.03), (PHENAZOPYRIDINE:Infectio n:0.12), (PHENAZOPYRIDINE HYDROCHLORIDE:Infection:0.28), (PHENTOLAMINE MESYLATE:Infection:0.1), (PHOSPHOCREATINE SODIUM:Infection:1), (PILOCARPINE HYDROCHLORIDE:Infection:0.1), (PILSICAINIDE HYDROCHLORIDE:Infection:0.45), (PIOGLITAZONE:Infection:0.65), (PLASMA PROTEIN FRACTION (HUMAN):Infection:0.13), (PLERIXAFOR:Infection:0.49), (PODOFILOX:Infection:0.1), (POLY ETHYLENE GLYCOL 3350\POTASSIUM CHLORIDESODIUM BICARBONATESODIUM CHLORIDESODIUM SULFATE ANHYDROUS:Infection:0.48), (POLYMYXIN:Infection:0.32), (POLYMYXIN B SULFATETRIMETHOPRIM SULFATE:Infection:0.29), (POLYSTYRENE SULFONIC ACID:Infection:0.21), (PORFIMER SODIUM:Infection:0.54), (POTASSIUM CITRATETRISODIUM CITRATE DIHYDRATE:Infection:1.09), (POTASSIUM IODIDE:Infection:0.69), (POTASSIUM PHOSPHATESODIUM PHOSPHATE:Infection:0.32), (POVIDONE:Infection:0.7), (PRAZOSIN HYDROCHLORIDE:Infection:0.96), (PYRIDOSTIGMINE BROMIDE:Infection:1.16), (QUINACRINE:Infection:0.48), (QUINAPRIL:Infection:0.68), (RAN IMUSTINE:Infection:0.75), (ROPIVACAINE HYDROCHLORIDE:Infection:0.53), (RUFINAMIDE:Infection:0.1), (SARPOGRELATE:Infecti on:1.04), (SELEGILINE:Infection:0.14), (SELEXIPAG:Infection:0.66), (SENNOSIDE B:Infection:0.22), (SODIUM CITRATE:Infection:1.12), (SODIUM FERROUS CITRATE:Infection:1.43), (SODIUM PHOSPHATE, DIBASIC, ANHYDROUSSODIUM PHOSPHATE, DIBASIC, HEPTAHYDRATESODIUM PHOSPHATE, MONOBASIC, MONOHYDRATE:Infection:0.42), (STARCH, WHEAT:Infection:0.01), (STREPTOMYCINSTREPTOMYCIN SULFATE:Infection:0.4), (SUPLATAST TOSILATE:Infection:0.38), (TABALUMAB:Infection:0.25), (TAFAMIDIS MEGLUMINE:Infection:0.11), (TAFLUPROST:Infection:0.09), (TAMOXIFEN CITRATE:Infection:0.2), (TEDUGLUTIDEWATER:Infection:0.52), (TEMSIROLIMUS:Infecti on:0.49), (TENELIGLIPTIN:Infection:0.02), (TENIPOSIDE:Infection:0.04), (TIANEPTINE:Infec tion:0.13), (TICLOPIDINE:Infection:0.58), (TIPEPIDINE HIBENZATE:Infection:0.04), (TISAGENLECLEUCEL:Infection:0.76), (TRIPTORELIN PAMOATE:Infection:0.11), (TROSPIUM CHLORIDE:Infection:1.62), (VINDESINE SULFATE:Infection:0.7), (VINORELBINEVINORELBINE TARTRATE:Infection:0.7), (ZAFIRLUKAST:Infection:0.88), (ZIV-AFLIBERCEPT:Infection:0.45), (ZUCLOPENTHIXOL DECANOATE:Infection:0.02), (.ALPHA.1-PROTEINASE INHIBITOR HUMAN:Infection prophylaxis:0.85), (ABOBOTULINUMTOXINA:Infection prophylaxis:0.32), (ACETAMINOPHENAPRONALIDECAFFEINEPROPYPHENAZONE:Inf ection prophylaxis:0.21), (ACETAMINOPHENCAFFEINE:Infection prophylaxis:0.59), (ACETAMINOPHENOXYCODONEOXYCODONE HYDROCHLORIDE:Infection prophylaxis:1.96), (ACETAMINOPHENPROPOXYPHENE HYDROCHLORIDE:Infection prophylaxis:0.83), (ACETAMINOPHENPROPOXYPHENE NAPSYLATE:Infection prophylaxis:0.87), (ADENINE:Infection prophylaxis:0.64), (ALBUTEROLGUAIFENESIN:Infection prophylaxis:0.06), (ALGLUCOSIDASE ALFA:Infection prophylaxis:0.51), (AMANTADINE:Infection prophylaxis:1.78), (AMINOSALICYLIC ACID:Infection prophylaxis:0.05), (AMLODIPINE BESYLATEATORVASTATIN CALCIUM:Infection prophylaxis:1.32), (AMLODIPINE BESYLATEHYDROCHLOROTHIAZIDEOLMESARTAN MEDOXOMIL:Infection prophylaxis:0.11), (AMMONIUM CHLORIDEDEXTROMETHORPHANSODIUM CITRATE:Infection prophylaxis:0.17), (AMMONIUM LACTATE:Infection prophylaxis:1.32), (AMOXAPINE:Infection prophylaxis:1.07), (ANAGLIPTIN:Infection prophylaxis:0.3), (ANAGRELIDE HYDROCHLORIDE:Infection prophylaxis:0.71), (ASCORBIC ACIDBIOTINCALCIUM PANTOTHENATECOBALAMINFOLIC ACIDNIACINAMIDEPYRIDOXINE HYDROCHLORIDERIBOFLAVINTHIAMINE MONONITRATE:Infection prophylaxis:0.16), (ASPIRINCLOPIDOGREL:Infection prophylaxis:0.48), (ATAZANAVIRRITONAVIR:Infection prophylaxis:0.05), (AVOBENZONEHOMOSALATEOCTISALATE:Infection prophylaxis:0.54), (AZELASTINE:Infection prophylaxis:0.84), (AZITHROMYCIN OR METHOTREXATE SODIUM:Infection prophylaxis:0.79), (BALSALAZIDE DISODIUM:Infection prophylaxis:0.81), (BECLOMETHASONEFORMOTEROL:Infection prophylaxis:0.29), (BENIDIPINE:Infection prophylaxis:0.36), (BENPROPERINE PHOSPHATE:Infection prophylaxis:0.26), (BEPRIDIL:Infection prophylaxis:1.5), (BETAMETHASONE DIPROPIONATECALCIPOTRIENE:Infection prophylaxis:0.43), (BETAMETHASONE DIPROPIONATECALCIPOTRIENE HYDRATE:Infection prophylaxis:0.07), (BETAMETHASONE DIPROPIONATECALCIPOTRIENECALCIPOTRIENE HYDRATE:Infection prophylaxis:0.42), (BETAMETHASONE VALERATEFUSIDIC ACID:Infection prophylaxis:0.66), (BETAMETHASONECALCIPOTRIENE:Infection prophylaxis:0.84), (BICARBONATE IONPOLYETHYLENE GLYCOLSPOTASSIUM CATIONSODIUM CHLORIDESODIUM SULFATE ANHYDROUS:Infection prophylaxis:0.2), (BIFIDOBACTERIUM LONGUM:Infection prophylaxis:0.82), (BIFIDOBACTERIUM LONGUM SUBSP. INFANTIS:Infection prophylaxis:0.76), (BILASTINE:Infection prophylaxis:1.03), (BIPERIDEN HYDROCHLORIDE:Infection prophylaxis:0.34), (BOCOCIZUMAB:Infection prophylaxis:0.12), (BOTULINUM TOXIN TYPE A:Infection prophylaxis:0.64), (BOTULISM ANTITOXIN:Infection prophylaxis:0.66), (BUDESONIDEFORMOTEROL FUMARATE:Infection prophylaxis:1.57), (BUFORMIN:Infection prophylaxis:0.01), (CABOZANTINIB:Infection prophylaxis:0.04), (CABOZANTINIB S-MALATE:Infection prophylaxis:0.13), (CALCIUM CARBONATEERGOCALCIFEROLRETINOL:Infection prophylaxis:1.46), (CALCIUMMAGNESIUMZINC:Infection prophylaxis:0.62), (CAMPHORDIPHENHYDRAMINEZINC OXIDE:Infection prophylaxis:0.12), (CANAGLIFLOZINMETFORMIN HYDROCHLORIDE:Infection prophylaxis:1.53), (CAPSAICIN:Infection prophylaxis:0.42), (CARBIDOPA:Infection prophylaxis:0.32), (CARTEOLOL:Infection prophylaxis:0.35), (CARVEDILOL PHOSPHATE:Infection prophylaxis:1.58), (CEFOPERAZONE SODIUMSULBACTAM:Infection prophylaxis:0.06), (CEFOXITINCEFOXITIN SODIUM:Infection prophylaxis:0.1), (CEFPROZIL:Infection prophylaxis:0.83), (CEFTAROLINE:Infection prophylaxis:0.06), (CEFTAZIDIME SODIUM:Infection prophylaxis:0.43), (CELIPROLOL:Infection prophylaxis:0.32), (CELLULASEDIASTASELIPASE:Infection prophylaxis:0.82), (CEPHARANTHINE:Infection prophylaxis:1.29), (CHLORAL HYDRATE:Infection prophylaxis:0.02), (CHLORPHENIRAMINE MALEATDIHYDROCODEINE PHOSPHATEMETHYLEPHEDRINE HYDROCHLORIDE, DL-:Infection prophylaxis:0.33), (CHOLINE BITARTRATECYSTEINEINOSITOLMAMMAL LIVER:Infection prophylaxis:0.26), (CHONDROITIN SULFATE (CHICKEN):Infection prophylaxis:0.98), (CICLESONIDE:Infection prophylaxis:1.46), (CICLOPIROX OLAMINE:Infection prophylaxis:0.04), (CIPROFIBRATE:Infection prophylaxis:0.22), (CLENBUTEROL HYDROCHLORIDE:Infection prophylaxis:0.98), (CLOFAZIMINE:Infection prophylaxis:0.32), (CLOPERASTINE:Infection prophylaxis:0.72), (COBICISTATELVITEGRAVIREMTRICITABINETENOFOVIR ALAFENAMIDE FUMARATE:Infection prophylaxis:0.22), (COLCHICINEDICYCLOMINE HYDROCHLORIDE:Infection prophylaxis:0.22), (COLISTIN SULFATE:Infection prophylaxis:0.08), (CORTICOTROPIN:Infection prophylaxis:0.85), (CROTALIDAE POLYVALENT IMMUNE FAB (OVINE):Infection prophylaxis:0.42), (CYAMEMAZINE:Infection prophylaxis:0.52), (CYANOCOBALAMINLIDOCAINE HYDROCHLORIDEPYRIDOXINE HYDROCHLORIDETHIAMINE HYDROCHLORIDE:Infection prophylaxis:0), (CYCLOPENTOLATE HYDROCHLORIDE:Infection prophylaxis:1.26), (CYTARABINE OCFOSFATE:Infection prophylaxis:0.2), (DABRAFENIB MESYLATE:Infection prophylaxis:0.4), (DAPAGLIFLOZIN PROPANEDIOLMETFORMIN HYDROCHLORIDE:Infection prophylaxis:0.15), (DASABUVIROMBITASVIRPARITAPREVIRRITONAVIR:Infection prophylaxis:0.23), (DAUNORUBICIN CITRATE:Infection prophylaxis:0.11), (DEFERIPRONE:Infection prophylaxis:0.45), (DENOPAMINE:Infection prophylaxis:0.24), (DEXTROAMPHETAMINE SULFATE:Infection prophylaxis:0.34), (DIAZOXIDE:Infection prophylaxis:0.2), (DIMEMORFAN:Infection prophylaxis:0.67), (DISOPYRAMIDE:Infection prophylaxis:0.47), (DOXEPIN HYDROCHLORIDE:Infection prophylaxis:0.77), (DOXYLAMINE SUCCINATE:Infection prophylaxis:0.82), (DUTASTERIDETAMSULOSIN HYDROCHLORIDE:Infection prophylaxis:0.52), (ECABET SODIUM:Infection prophylaxis:0.73), (EMPAGLIFLOZINLINAGLIPTIN:Infection prophylaxis:1.27), (EPALRESTAT:Infection prophylaxis:0.01), (EPHEDRA SINICA STEMHERBALS:Infection prophylaxis:0.4), (ERIBULIN MESYLATE:Infection prophylaxis:0.6), (ESTROGENS:Infection prophylaxis:0.47), (ESTROGENS, CONJUGATEDMEDROXYPROGESTERONE ACETATE:Infection prophylaxis:1.1), (ETHACRYNIC ACID:Infection prophylaxis:0.38), (ETHINYL ESTRADIOLETHYNODIOL:Infection prophylaxis:0.09), (ETHINYL ESTRADIOLNORGESTREL:Infection prophylaxis:0.35), (ETHYL ICOSAPENTATEICOSAPENT:Infection prophylaxis:1.18), (ETRETINATE:Infection prophylaxis:2.24), (EVOLOCUMAB:Infection prophylaxis:1.62), (FENOFIBRIC ACID:Infection prophylaxis:0.71), (FERRIC CITRATE ANHYDROUS:Infection prophylaxis:0.4), (FERRIC HYDROXIDE:Infection prophylaxis:0.05), (FERRIC PYROPHOSPHATE:Infection prophylaxis:0.73), (FERROUS CITRATESODIUM:Infection prophylaxis:0.5), (FERROUS GLYCINE SULFATE:Infection prophylaxis:0.41), (FERROUS SULFATE ANHYDROUS:Infection prophylaxis:0.78), (FERROUS SULFATEGLYCINE:Infection prophylaxis:1.35), (FLAVIN ADENINE DINUCLEOTIDE:Infection prophylaxis:0.97), (FLAVOXATE HYDROCHLORIDE:Infection prophylaxis:0.07), (FLOMOXEF:Infection prophylaxis:0.5), (FLUOROURACILLEUCOVORIN CALCIUMOXALIPLATIN:Infection prophylaxis:0.32), (FLUTAMIDE:Infection prophylaxis:0.57), (FORMOTEROL FUMARATEMOMETASONE FUROATE:Infection prophylaxis:1.19), (FOSFOMYCIN CALCIUM:Infection prophylaxis:0.39), (FOSFOMYCIN SODIUM:Infection prophylaxis:0.35), (FUDOSTEINE:Infection prophylaxis:1.07), (FURSULTIAMINE:Infection prophylaxis:0.71), (GADOBENATE DIMEGLUMINE:Infection prophylaxis:0.76), (GADOTERATE MEGLUMINE:Infection prophylaxis:0.08), (GANCICLOVIR SODIUM:Infection prophylaxis:0.22), (GEFITINIB:Infection prophylaxis:0.72), (GLIPIZIDEMETFORMIN HYDROCHLORIDE:Infection prophylaxis:0.12), (GLYCOPYRROLATEINDACATEROL MALEATE:Infection prophylaxis:0.57), (GRANULOCYTE-MACROPHAGE COLONY-STIMULATING FACTOR:Infection prophylaxis:0.11), (HYALURONIC ACID:Infection prophylaxis:0.09), (HYDROCODONE BITARTRATEIBUPROFEN:Infection prophylaxis:0.17), (HYDROCORTISONE ACETATE:Infection prophylaxis:1.79), (HYDROCORTISONE PROBUTATE:Infection prophylaxis:0.3), (HYDROXOCOBALAMINPYRIDOXINE HYDROCHLORIDETHIAMINE HYDROCHLORIDE:Infection prophylaxis:0.2), (HYDROXYCHLOROQUINE DIPHOSPHATE:Infection prophylaxis:0.77), (HYOSCYAMINE SULFATE:Infection prophylaxis:0.21), (IDARUCIZUMAB:Infection prophylaxis:0.37), (IFENPRODIL:Infection prophylaxis:0.37), (ILOPROST:Infection prophylaxis:1.27), (IMIDAFENACIN:Infection prophylaxis:0.52), (IMIDAPRIL HYDROCHLORIDE:Infection prophylaxis:0.42), (IMIGLUCERASE:Infection prophylaxis:0.69), (IMIPRAMINE HYDROCHLORIDE:Infection prophylaxis:0.81), (INFLIXIMAB-DYYB:Infection prophylaxis:0.61), (INSULIN PORKINSULIN PURIFIED PORK:Infection prophylaxis:0.07), (INTERFERON:Infection prophylaxis:1.76), (INTERFERON ALFA-2A:Infection prophylaxis:1.47), (INTERFERON ALFA-N1:Infection prophylaxis:0.15), (INTERFERON BETA:Infection prophylaxis:0.1), (INTERFERON GAMMA:Infection prophylaxis:0.01), (INTERFERON GAMMA-1B:Infection prophylaxis:1.48), (IPRAGLIFLOZIN L-PROLINE:Infection prophylaxis:0.08), (IXABEPILONE:Infection prophylaxis:0.25), (LACTASE:Infection prophylaxis:0.74), (LACTOBACILLUS ACIDOPHILUSLACTOBACILLUS DELBRUECKII SUBSP. BULGARICUS:Infection prophylaxis:0.5), (LANREOTIDE ACETATE:Infection prophylaxis:0.92), (LAPATINIB DITOSYLATE:Infection prophylaxis:0.63), (LENVATINIB:Infection prophylaxis:0.57), (LEVOMEFOLATE CALCIUMMETHYLCOBALAMINPYRIDOXAL PHOSPHATE ANHYDROUS:Infection prophylaxis:0.19), (LEVOSIMENDAN:Infection prophylaxis:0.24), (LINSIDOMINE:Infection prophylaxis:0.22), (LIOTHYRONINE:Infection prophylaxis:0.53), (LITHIUM:Infection prophylaxis:0.72), (LOMITAPIDE MESYLATE:Infection prophylaxis:0.31), (MAGNESIUM SULFATE HEPTAHYDRATE:Infection prophylaxis:0.02), (MAPROTILINE HYDROCHLORIDE:Infection prophylaxis:0.09), (MELPHALAN HYDROCHLORIDE:Infection prophylaxis:0.4), (MEPENZOLATE BROMIDE:Infection prophylaxis:0.11), (METAXALONE:Infection prophylaxis:1.69), (METFORMIN HYDROCHLORIDESAXAGLIPTIN:Infection prophylaxis:0.14), (METFORMIN PAMOATE:Infection prophylaxis:0.57), (METHOTREXATEMETHOTREXATE SODIUM:Infection prophylaxis:0.49), (METHYLNALTREXONE:Infection prophylaxis:0.12), (METHYLPREDNISOLONE HEMISUCCINATE:Infection prophylaxis:0.11), (METHYLPREDNISOLONE OR METHYLPREDNISOLONE ACETATE:Infection prophylaxis:0.32), (METOPIMAZINE:Infection prophylaxis:0.03), (METYRAPONE:Infection prophylaxis:0.17), (MEXILETINE:Infection prophylaxis:0.14), (MIGLUSTAT:Infection prophylaxis:0.71), (MITIGLINIDE CALCIUM HYDRATEVOGLIBOSE:Infection prophylaxis:0.3), (MOEXIPRIL HYDROCHLORIDE:Infection prophylaxis:0.63), (NABIXIMOLS:Infection prophylaxis:0.23), (NALOXONE HYDROCHLORIDETILIDINE HYDROCHLORIDE:Infection prophylaxis:1.89), (NARATRIPTAN HYDROCHLORIDE:Infection prophylaxis:0.12), (NATEGLINIDE:Infection prophylaxis:1.38), (NICERGOLINE:Infection prophylaxis:0.09), (NILVADIPINE:Infection prophylaxis:0.01), (NORTRIPTYLINE:Infection prophylaxis:1.68), (OK-432:Infection prophylaxis:0.23), (OLODATEROL HYDROCHLORIDETIOTROPIUM BROMIDE MONOHYDRATE:Infection prophylaxis:1.92), (OPIPRAMOL:Infection prophylaxis:0.13), (ORGOTEIN:Infection prophylaxis:0.03), (ORPHENADRINE:Infection prophylaxis:0.53), (OXYBUTYNINOXYBUTYNIN CHLORIDE:Infection prophylaxis:0.04), (PANCURONIUM:Infection prophylaxis:0.21), (PARATHYROID HORMONE:Infection prophylaxis:0.13), (PARICALCITOL:Infection prophylaxis:0.93), (PEGINTERFERON BETA-1A:Infection prophylaxis:1.09), (PEGVISOMANT:Infection prophylaxis:0.24), (PENTOBARBITAL:Infection prophylaxis:0.25), (PENTOSTATIN:Infection prophylaxis:2.2), (PHOSPHORUS:Infection prophylaxis:0.42), (PILSICAINIDE:Infection prophylaxis:0.04), (PIMECROLIMUS:Infection prophylaxis:0.45), (PIMOBENDAN:Infection prophylaxis:1.57), (PIRENZEPINE:Infection prophylaxis:0.58), (PIROXICAM:Infection prophylaxis:0.95), (PLASMA PROTEIN FRACTION (HUMAN):Infection prophylaxis:0.42), (POLYMYXIN:Infection prophylaxis:0.7), (POLYSTYRENE:Infection prophylaxis:0.5), (POLYSTYRENE SULFONIC ACID:Infection prophylaxis:1.11), (PORFIMER SODIUM:Infection prophylaxis:0.8), (POTASSIUM PHOSPHATE, MONOBASICSODIUM PHOSPHATE, DIBASIC, ANHYDROUSSODIUM PHOSPHATE, MONOBASIC, MONOHYDRATE:Infection prophylaxis:0.05), (PRAMIPEXOLE DIHYDROCHLORIDE:Infection prophylaxis:1.54), (PREDNISOLONE VALERATE ACETATE:Infection prophylaxis:0.33), (PYRIDOXAL PHOSPHATE:Infection prophylaxis:0.92), (QUINACRINE:Infection prophylaxis:0.9), (RISEDRONIC ACID:Infection prophylaxis:0.12), (ROXATIDINE ACETATE HYDROCHLORIDE:Infection prophylaxis:0.28), (SACUBITRILVALSARTAN:Infection prophylaxis:0.76), (SARPOGRELATE:Infection prophylaxis:2.41), (SARPOGRELATE HYDROCHLORIDE:Infection prophylaxis:0.76), (SELEXIPAG:Infection prophylaxis:0.52), (SENNOSIDE B:Infection prophylaxis:0.63), (SERRAPEPTASE:Infection prophylaxis:0.32), (SIPULEUCEL-T:Infection prophylaxis:0.44), (SITAFLOXACIN:Infection prophylaxis:0.53), (SOBUZOXANE:Infection prophylaxis:0.2), (SOFOSBUVIRVELPATASVIR:Infection prophylaxis:0.07), (SORBITOL:Infection prophylaxis:0.64), (TAFAMIDIS MEGLUMINE:Infection prophylaxis:0.09), (TAFLUPROST:Infection prophylaxis:0.65), (TAMOXIFEN CITRATE:Infection prophylaxis:0.46), (TEDUGLUTIDEWATER:Infection prophylaxis:0.47), (TENIPOSIDE:Infection prophylaxis:0.43), (TETRACAINE HYDROCHLORIDE:Infection prophylaxis:0.61), (TETRACAINETETRACAINE HYDROCHLORIDE:Infection prophylaxis:0.48), (THIAMYLAL:Infection prophylaxis:0.56), (THROMBOPOIETIN:Infection prophylaxis:1.73), (TOCOPHERYL NICOTINATE, D-.ALPHA.:Infection prophylaxis:0.96), (TOPIROXOSTAT:Infection prophylaxis:0.08), (TRABECTEDIN:Infection prophylaxis:0.59), (TRAMETINIB DIMETHYL SULFOXIDE:Infection prophylaxis:0.52), (TRIHEXYPHENIDYL:Infection prophylaxis:0.29), (TROXIPIDE:Infection prophylaxis:0.47), (TULOBUTEROL HYDROCHLORIDE:Infection prophylaxis:0.16), (UMECLIDINIUM BROMIDE:Infection prophylaxis:0.9), (UMECLIDINIUM BROMIDEVILANTEROL TRIFENATATE:Infection prophylaxis:1.49), (VANDETANIB:Infection prophylaxis:0.06), (VONOPRAZAN:Infection prophylaxis:0.52), (YEAST:Infection prophylaxis:0.24), (ZAFIRLUKAST:Infection prophylaxis:0.63), (ZICONOTIDE ACETATE:Infection prophylaxis:0.09), (ZILEUTON:Infection prophylaxis:0.59), (ZIV-AFLIBERCEPT:Infection prophylaxis:0.65), (LUMACAFTOR:Infective pulmonary exacerbation of cystic fibrosis:0.12), (NAFARELIN ACETATE:Infertility:0.55), (BUSERELIN:Infertility female:0.21), (CETRORELIX ACETATE:Infertility female:0.58), (ATRACURIUM:Infiltration anaesthesia:0.5), (SUCCINYLCHOLINE CHLORIDE:Infiltration anaesthesia:0.06), (SUGAMMADEX:Infiltration anaesthesia:0.45), (.ALPHA.1-PROTEINASE INHIBITOR HUMAN:Inflammation:1.12), (ABOBOTULINUMTOXINA:Inflammation:0.02), (ACETAMIN OPHENCAFFEINE:Inflammation:0.08), (ACETAMINOPHENDIPHENHYDRAMINE HYDROCHLORIDE:Inflammation:0.28), (ACTIVATED CHARCOAL:Inflammation:0.08), (AGALSIDASE BETA:Inflammation:1.03), (ALBIGLUTIDE:Inflammation:0.18), (ALDESLEUKIN:Inflammatio n:0.25), (AMILORIDE HYDROCHLORIDEHYDROCHLOROTHIAZIDE:Inflammation:0.52), (AMINO ACIDSDEXTROSEELECTROLYTES NOS:Inflammation:0.32), (AMISULPRIDE:Inflammation:1.07), (AMLODIPINE BESYLATEHYDROCHLOROTHIAZIDEVALSARTAN:Inflammation:0.83), (AMLODIPINE VALSARTAN:Inflammation:1.08), (AMMONIUM CHLORIDEDEXTROMETHORPHANSODIUM CITRATE:Inflammation:0.28), (AMOXICILLINCLARITHROMYCINESOMEPRAZOLE:Infl ammation:0.37), (ANTIHISTAMINES NOS:Inflammation:0.68), (ARIPIPRAZOLE LAUROXIL:Inflammation:0.09), (ASPIRINDIHYDROXYALUMINUM AMINOACETATE ANHYDROUSMAGNESIUM CARBONATE:Inflammation:0.05), (ATOVAQUONE:Inflammation:1.21), (ATRACURIUM:Infl ammation:0.13), (BAMIFYLLINE HYDROCHLORIDE:Inflammation:0.03), (BECLOMETHASONE:Inflammation:0.34), (BECLO METHASONEFORMOTEROL:Inflammation:0.16), (BELATACEPT:Inflammation:0.27), (BET AMETHASONECALCIPOTRIENE:Inflammation:0.64), (BETHANECHOL CHLORIDE:Inflammation:0.19), (BLINATUMOMAB:Inflammation:0.08), (BRINZOLAMIDE TIMOLOL MALEATE:Inflammation:0.14), (CABAZITAXEL:Inflammation:0.04), (CABOZANTINIB S-MALATE:Inflammation:0.18), (CALCITONIN:Inflammation:0.77), (CALCIUM CARBONATEERGOCALCIFEROLRETINOL:Inflammation:0.64), (CAMPHORDIPHENHY DRAMINEZINC OXIDE:Inflammation:0), (CANAGLIFLOZINMETFORMIN HYDROCHLORIDE:Inflammation:1.6), (CARBIDOPA:Inflammation:0.02), (CARBIMAZOLE: Inflammation:0.38), (CARVEDILOL PHOSPHATE:Inflammation:2.01), (CASANTHRANOLDOCUSATE SODIUM:Inflammation:0.73), (CASPOFUNGIN:Inflammation:0.14), (CEFOPERAZONE:Infla mmation:0.28), (CHLORAMBUCIL:Inflammation:0.38), (CISATRACURIUM:Inflammation:0.38), (CLIKSTAR:Inflammation:0.12), (CLOBAZAM:Inflammation:0.46), (CLOSTRIDIUM BUTYRICUM SPORES STRAIN M-55:Inflammation:0.28), (COLISTIMETHATE SODIUM:Inflammation:0.2), (DABRAFENIB:Inflammation:0.25), (DABRAFENIB MESYLATE:Inflammation:0.04), (DACLIZUMAB:Inflammation:1.94), (DANAZOL:Inflammati on:0.79), (DANTROLENE SODIUM:Inflammation:0.35), (DASATINIB:Inflammation:2.11), (DESFLURANE:Inflammation:0.89), (DEXAMETHASONE ACETATE:Inflammation:0.46), (DEXTROAMPHETAMINE SULFATE:Inflammation:1.12), (DIMETHICONESILICON DIOXIDESIMETHICONE:Inflammation:0.4), (DIPYRIDAMOLE:Inflammation:0.89), (DOBU TAMINEDOBUTAMINE HYDROCHLORIDE:Inflammation:0.85), (DOLASETRON MESYLATE:Inflammation:0.55), (ECONAZOLE:Inflammation:0.71), (EDOXABAN TOSYLATE:Inflammation:1.11), (ELOTUZUMAB:Inflammation:0.95), (EMPAGLIFLOZINLI NAGLIPTIN:Inflammation:1.48), (ERIBULIN:Inflammation:0.35), (ERTAPENEM SODIUM:Inflammation:0.02), (ESMOLOL HYDROCHLORIDE:Inflammation:0.86), (ESTRIOL:Inflammation:0.12), (FENOTEROL HYDROBROMIDE:Inflammation:0.41), (FENOTEROL HYDROBROMIDEIPRATROPIUM BROMIDE:Inflammation:0.26), (FERROUS SULFATE ANHYDROUS:Inflammation:0.29), (FERROUS SULFATEGLYCINE:Inflammation:0.61), (FLUOCINOLONE ACETONIDE:Inflammation:1.13), (FLURAZEPAMFLURAZEPAM HYDROCHLORIDE:Inflammation:0.32), (FONDAPARINUX:Inflammation:0.33), (FONDAPA RINUX SODIUM:Inflammation:0.35), (FORMOTEROL:Inflammation:0.9), (FOSFOMYCIN:Inflammati on:0.05), (FROVATRIPTAN SUCCINATE:Inflammation:0.09), (GLIPIZIDEMETFORMIN HYDROCHLORIDE:Inflammation:0.14), (GLYCOPYRROLATEINDACATEROL MALEATE:Inflammation:0.4), (GLYCOPYRRONIUMINDACATEROL:Inflammation:1.45), (GUANFACINE:Inflammation:0.84), (GUANFACINE HYDROCHLORIDE:Inflammation:0.58), (HYALURONIC ACID:Inflammation:0.03), (HYDROCHLOROTHIAZIDETELMISARTAN:Inflammation:0.91), (HYDROCODONE BITARTRATEIBUPROFEN:Inflammation:0.87), (HYDROCORTISONE ACETATE:Inflammation:1.23), (HYOSCYAMINE SULFATEMETHENAMINEMETHYLENE BLUEPHENYL SALICYLATESODIUM PHOSPHATE, MONOBASIC, MONOHYDRATE:Inflammation:0.08), (IDELALISIB:Inflammation:0.37), (IMATINIB MESYLATE:Inflammation:1.5), (IMIQUIMOD:Inflammation:0.17), (IMMUNE GLOBULIN NOS:Inflammation:0.19), (INDACATEROL:Inflammation:0.69), (INSULIN DEGLUDEC:Inflammation:0.83), (INTERFERON:Inflammation:0.46), (INTERFERON ALFA-2A:Inflammation:0.93), (INTERFERON GAMMA-1B:Inflammation:0.42), (IRINOTECAN:Inflammation:0.5), (IRINOTECAN HYDROCHLORIDE:Inflammation:0.66), (IXAZOMIB:Inflammation:0.52), (LACTOBACILLU S RHAMNOSUS:Inflammation:1.16), (LAPATINIB DITOSYLATE:Inflammation:0.58), (LENVATINIB:Inflammation:0.14), (LERCANIDIPINE:Infl ammation:2.1), (LEVOTHYROXINE SODIUMPOTASSIUM IODIDE:Inflammation:0.23), (LORNOXICAM:Inflammation:0.04), (MEDROXYPROGESTER ONE:Inflammation:0.39), (METHENOLONE ENANTHATE:Inflammation:0.09), (MICAFUNGIN:Inflammation:0.17), (MIDODRINE HYDROCHLORIDE:Inflammation:0.78), (MOEXIPRIL HYDROCHLORIDE:Inflammation:0.38), (NALOXONE:Inflammation:0.75), (NALOXONE HYDROCHLORIDEOXYCODONE HYDROCHLORIDE:Inflammation:1.71), (NEBIVOLOL:Inflammation:1.38), (NEOSTIGMINE: Inflammation:0.82), (NILOTINIB:Inflammation:2.2), (OMEPRAZOLESODIUM BICARBONATE:Inflammation:0.05), (OXYBUTYNINOXYBUTYNIN CHLORIDE:Inflammation:0.36), (PARICALCITOL:Inflammation:0.31), (PATIROMER:Inflam mation:0.39), (PEGINTERFERON BETA-1A:Inflammation:1.18), (POLYMYXIN B SULFATETRIMETHOPRIM SULFATE:Inflammation:0.29), (PONATINIB:Inflammation:1.43), (POVIDONE:Inflammation:0.38), (PRAMLINTIDE ACETATE:Inflammation:0.01), (PRANLUKAST:Inflammation:0.05), (PROPAFENONE:Inflam mation:0.22), (PROTRIPTYLINE HYDROCHLORIDE:Inflammation:0.58), (RANOLAZINE:Inflammation:1.94), (REGORAFENI B:Inflammation:1.11), (RETINOL:Inflammation:1.29), (RIVASTIGMINE:Inflammation:0.66), (R IVASTIGMINE TARTRATE:Inflammation:0.51), (SALMETEROL XINAFOATE:Inflammation:0.76), (SEVELAMER HYDROCHLORIDE:Inflammation:0.27), (SODIUM CITRATE:Inflammation:0.34), (STARCH, WHEAT:Inflammation:0.16), (TAMOXIFEN CITRATE:Inflammation:0.46), (TASIMELTEON:Inflammation:0.15), (TAZOBACTAM:Inflam mation:0.36), (TEDUGLUTIDEWATER:Inflammation:0.15), (TERCONAZOLE:Inflammation: 0.03), (TESTOSTERONE UNDECANOATE:Inflammation:0.4), (TETRACYCLINE:Inflammation:1.91), (THEOPHYLLIN E:Inflammation:0.49), (THIOPENTAL:Inflammation:0.28), (TIMOLOLTRAVOPROST:Inflam mation:0), (TINZAPARIN:Inflammation:0.29), (TIPIRACIL HYDROCHLORIDETRIFLURIDINE:Inflammation:0.21), (TRAMETINIB:Inflammation:0.18), (TRAMETINIB DIMETHYL SULFOXIDE:Inflammation:0.05), (TRIMIPRAMINE MALEATE:Inflammation:0.15), (TRIPTORELIN PAMOATE:Inflammation:0.17), (TROSPIUM CHLORIDE:Inflammation:0.76), (UMECLIDINIUM BROMIDE:Inflammation:0.86), (VARDENAFIL HYDROCHLORIDE TRIHYDRATE:Inflammation:0.27), (VECURONIUM BROMIDE:Inflammation:0.67), (ZILEUTON:Inflammation:0.48), (ALBUTEROL SULFATEIPRATROPIUM BROMIDE:Inflammatory bowel disease:0.35), (AMINO ACIDSDEXTROSEELECTROLYTES NOS:Inflammatory bowel disease:0.19), (AMIODARONE:Inflammatory bowel disease:0.19), (ANASTROZOLE:Inflammatory bowel disease:0.1),(ARMODAFINIL:Inflammatory bowel disease:0.64), (ATORVASTATIN CALCIUM:Inflammatory bowel disease:0.8), (BALSALAZIDE DISODIUM:Inflammatory bowel disease:0.08), (BUMETANIDE:Inflammatory bowel disease:0.5), (BUPRENORPHINE HYDROCHLORIDENALOXONE HYDROCHLORIDE:Inflammatory bowel disease:0.5), (CALCIUM ACETATE:Inflammatory bowel disease:0.36), (CANAKINUMAB:Inflammatory bowel disease:0.12), (CANDESARTAN CILEXETIL:Inflammatory bowel disease:0.04), (CARISOPRODOL:Inflammatory bowel disease:0.18), (CEFTRIAXONE SODIUM:Inflammatory bowel disease:0.31), (CHONDROITIN SULFATE (BOVINE)GLUCOSAMINE HYDROCHLORIDE:Inflammatory bowel disease:0.02), (CLOTRIMAZOLE:Inflammatory bowel disease:0.13), (COLCHICINE:Inflammatory bowel disease:0.48), (CYCLOBENZAPRINE HYDROCHLORIDE:Inflammatory bowel disease:0.78), (DIMENHYDRINATE:Inflammatory bowel disease:0.04), (ELETRIPTAN HYDROBROMIDE:Inflammatory bowel disease:0.58), (ESOMEPRAZOLE MAGNESIUMNAPROXEN:Inflammatory bowel disease:0.16), (ESTROGENS, CONJUGATED:Inflammatory bowel disease:0.11), (EVEROLIMUS:Inflammatory bowel disease:0.05), (EVOLOCUMAB:Inflammatory bowel disease:0.42), (EZETIMIBE:Inflammatory bowel disease:0.03), (FENOFIBRATE:Inflammatory bowel disease:0.18), (HYDROXYCHLOROQUINE DIPHOSPHATE:Inflammatory bowel disease:0.35), (HYDROXYZINE PAMOATE:Inflammatory bowel disease:0.47), (IPRATROPIUM:Inflammatory bowel disease:0.11), (LOVASTATIN:Inflammatory bowel disease:0.3), (METAXALONE:Inflammatory bowel disease:0.8), (NADOLOL:Inflammatory bowel disease:0.08), (NAPROXEN SODIUM:Inflammatory bowel disease:0.33), (NITROFURANTOIN:Inflammatory bowel disease:0.29), (OLANZAPINE:Inflammatory bowel disease:0.1), (OXYGEN:Inflammatory bowel disease:0.25), (PANTOPRAZOLE MAGNESIUM:Inflammatory bowel disease:0.4), (PAROXETINE:Inflammatory bowel disease:0), (PAROXETINEPAROXETINE HYDROCHLORIDE:Inflammatory bowel disease:0.01), (PRIMIDONE:Inflammatory bowel disease:0.03), (PYRIDOXINE:Inflammatory bowel disease:0.1), (PYRIDOXINE HYDROCHLORIDE:Inflammatory bowel disease:0.02), (QUETIAPINE:Inflammatory bowel disease:0.02), (RABEPRAZOLE SODIUM:Inflammatory bowel disease:0), (RISEDRONATE SODIUM:Inflammatory bowel disease:0.34), (ROPINIROLE HYDROCHLORIDE:Inflammatory bowel disease:0.05), (TEMAZEPAM:Inflammatory bowel disease:0.51), (TERIFLUNOMIDE:Inflammatory bowel disease:0.31), (TETRACYCLINE:Inflammatory bowel disease:0.03), (TIZANIDINE HYDROCHLORIDE:Inflammatory bowel disease:0.39), (TOCILIZUMAB:Inflammatory bowel disease:0.67), (TORSEMIDE:Inflammatory bowel disease:0.27), (UMECLIDINIUM BROMIDEVILANTEROL TRIFENATATE:Inflammatory bowel disease:0.37), (VALGANCICLOVIR HYDROCHLORIDE:Inflammatory bowel disease:0.1), (VARENICLINE TARTRATE:Inflammatory bowel disease:0.76), (BUPROPION HYDROCHLORIDE:Inflammatory pain:0.11), (ENALAPRIL:Inflammatory pain:0.05), (ESCITALOPRAM OXALATE:Inflammatory pain:0.09), (EVOLOCUMAB:Inflammatory pain:0.07), (.ALPHA.1-PROTEINASE INHIBITOR HUMAN:Influenza:1.03), (ACETAMINOPHENOXYCODONE HYDROCHLORIDE:Influenza:1.32), (ACETAMINOPHENOXYCODONEOXYCODONE HYDROCHLORIDE:Influenza:0.42), (ACETAMINOPHENPROPOXYPHENE HYDROCHLORIDE:Influenza:0.08), (AGALSIDASE BETA:Influenza:0.09), (ALENDRONIC ACID:Influenza:0.74), (ALUMINUM HYDROXIDEDIMETHICONEMAGNESIUM HYDROXIDE:Influenza:0.37), (AMISULPRIDE:Influenza:0.44), (AMLODIPINEVALSARTA N:Influenza:0.15), (AMOXAPINE:Influenza:0.17), (AZELASTINE HYDROCHLORIDE:Influenza:0.23), (BECLOMETHASONEFORMOTEROL:Influenza:0.15), (BETAMETHASONE DIPROPIONATE:Influenza:0.51), (BRINZOLAMIDE:Influenza:0.52), (BUPRENORPHINE HYDROCHLORIDENALOXONE HYDROCHLORIDE:Influenza:0.67), (CALCIUM ACETATE:Influenza:0.72), (CALCIUM POLYCARBOPHIL:Influenza:0.09), (CANAGLIFLOZINMETFORMIN HYDROCHLORIDE:Influenza:0.24), (CANAKINUMAB:Influenza:0.72), (CARISOPRODOL:I nfluenza:0.47), (CHLORTHALIDONE:Influenza:0.81), (CIMETIDINE:Influenza:0.92), (CLOMI PRAMINE HYDROCHLORIDE:Influenza:0.44), (COD LIVER OIL:Influenza:0.05), (COLESEVELAM HYDROCHLORIDE:Influenza:0.11), (COLISTIN:Influenza:0.18), (CORTICOTROPIN:Influenz a:0.2), (CRIZOTINIB:Influenza:0.08), (DABIGATRAN:Influenza:0.32), (DACLIZUMAB:Influe nza:0.96), (DALTEPARIN:Influenza:0.61), (DASATINIB:Influenza:1.35), (DEVICE:Influenza:0.22), (DEXAMETHASONE ACETATE:Influenza:0.61), (DILAZEP:Influenza:0.3), (DOCUSATE:Influenza:1.06), (DRONAB INOL:Influenza:0.52), (ECONAZOLE:Influenza:0.3), (ELETRIPTAN HYDROBROMIDE:Influenza:1.22), (ELOTUZUMAB:Influenza:0.35), (ESMOLOL HYDROCHLORIDE:Influenza:0.05), (FENOTEROL HYDROBROMIDEIPRATROPIUM BROMIDE:Influenza:0.55), (FERROUS GLUCONATE:Influenza:0.14), (FERROUS SULFATEGLYCINE:Influenza:0.17), (FLAVIN ADENINE DINUCLEOTIDE:Influenza:0.14), (FLUVOXAMINE MALEATE:Influenza:0.09), (FORMOTEROL:Influenza:0.77), (FORMOTEROL FUMARATE:Influenza:1.5), (FOSFOMYCIN:Influenza:0.1), (FOSPHENYTOINFOSPHENYT OIN SODIUM:Influenza:0.24), (FULVESTRANT:Influenza:0.57), (FUSIDIC ACID:Influenza:0.55), (HERBALSTURMERIC:Influenza:0.08), (HYDROCHLOROTHIAZIDE TELMISARTAN:Influenza:0.57), (HYDROXYCHLOROQUINE DIPHOSPHATE:Influenza:0.24), (HYOSCYAMINE:Influenza:0.27), (ICOSAPENT ETHYL:Influenza:0.17), (ILOPROST:Influenza:1.11), (IMATINIB MESYLATE:Influenza:0.73), (IMIPENEM:Influenza:0.5), (IMIPRAMINE HYDROCHLORIDE:Influenza:0.94), (INDAPAMIDEPERINDOPRIL:Influenza:0.98), (INTER FERON GAMMA-1B:Influenza:0.37), (KETOROLACKETOROLAC TROMETHAMINE:Influenza:0.05), (LACTOBACILLUS RHAMNOSUS:Influenza:0.25), (LIDOCAINEPRILOCAINE:Influenza:0.56), (LUMACAFTOR:Influenza:0.24), (METAXALONE:Influenza:0.8), (METHIMAZOLE:Influenza:0.34), (MIDODR INE:Influenza:0.09), (MIDODRINE HYDROCHLORIDE:Influenza:0.08), (MINOCYCLINEMINOCYCLINE HYDROCHLORIDE:Influenza:0.78), (MOXONIDINE:Influenza:0.12), (NABUMETONE:Influe nza:0.45), (NADOLOL:Influenza:0.27), (NADROPARIN CALCIUM:Influenza:0.05), (NATEGLINIDE:Influenza:0.25), (NICORANDIL:Influenza:0.19), (NOREPINEPHRINE:Influenza:0.28), (NOREPINEPHRINE BITARTRATE:Influenza:0.05), (NORETHINDRONE:Influenza:0.18), (OLODATEROL HYDROCHLORIDETIOTROPIUM BROMIDE MONOHYDRATE:Influenza:1.26), (OLOPATADINE:Influenza:0.57), (PANTOPRAZOLE MAGNESIUM:Influenza:1.84), (PAZOPANIB HYDROCHLORIDE:Influenza:0.91), (PEGINTERFERON BETA-1A: Influenza:0.68), (PENICILLIN V:Influenza:0.2), (PILSICAINIDE HYDROCHLORIDE:Influenza:0.32), (PIMOBENDAN:Influenza:0.18), (PIROXICAM:Influenza:0.37), (PONATINIB:Influenza:0.5), (PRAZOSIN HYDROCHLORIDE:Influenza:0.09), (PROPAFENONE HYDROCHLORIDE:Influenza:0.07), (RANOLAZINE:Influenza:1.32), (RETINOL:Influenza:0.3 9), (RIVASTIGMINE TARTRATE:Influenza:0.17), (RIZATRIPTAN:Influenza:0.09), (ROFLUMILAST:Influenza:1.12), (ROPINIROLE:Influenza:0.34), (SACUBITRILVALSARTAN:Influenza:1.16), (SCOPOLAMI NE:Influenza:0.88), (SODIUM CITRATE:Influenza:0.05), (SUMATRIPTAN:Influenza:0.57), (SUPLATAST TOSILATE:Influenza:0.35), (TETRACYCLINE:Influenza:0.58), (THEOPHYLLINE:Influenza:0. 34), (TIGECYCLINE:Influenza:0.48), (TIMOLOL:Influenza:0.53), (UMECLIDINIUM BROMIDE:Influenza:0.42), (VILAZODONE HYDROCHLORIDE:Influenza:0.32), (VITAMIN A:Influenza:0.59), (ZAFIRLUKAST:Influenza:0.22), (ZILEUTON:Influenza:0.31), (AMPHETA MINE ASPARTATEAMPHETAMINE SULFATEDEXTROAMPHETAMINE SACCHARATEDEXTROAMPHETAMINE SULFATE:Influenza like illness:0.12), (ANASTROZOLE:Influenza like illness:0.66), (BUMETANIDE:Influenza like illness:0.27), (BUPROPION HYDROCHLORIDE:Influenza like illness:1.11), (BUSPIRONE HYDROCHLORIDE:Influenza like illness:0.12), (CALCIUM CARBONATE:Influenza like illness:0.14), (CALCIUM CARBONATECHOLECALCIFEROL:Influenza like illness:0.55), (CALCIUMVITAMIN D:Influenza like illness:0.02), (CERTOLIZUMAB PEGOL:Influenza like illness:0.51), (CLOPIDOGREL BISULFATE:Influenza like illness:0.22), (COLCHICINE:Influenza like illness:0.12), (DEXLANSOPRAZOLE:Influenza like illness:0.78), (DIGOXIN:Influenza like illness:0.08), (DILTIAZEM:Influenza like illness:0.57), (DILTIAZEM HYDROCHLORIDE:Influenza like illness:0.54), (DULOXETINE:Influenza like illness:0.17), (ESZOPICLONE:Influenza like illness:0.27), (EVEROLIMUS:Influenza like illness:0.02), (EVOLOCUMAB:Influenza like illness:0.12), (HYDROMORPHONE HYDROCHLORIDE:Influenza like illness:0.05), (IMATINIB MESYLATE:Influenza like illness:0.15), (INDOMETHACIN:Influenza like illness:0.07), (INSULIN ASPART:Influenza like illness:0.04), (ISOSORBIDE MONONITRATE:Influenza like illness:0.32), (OLANZAPINE:Influenza like illness:0.42), (OXCARBAZEPINE:Influenza like illness:0.22), (PRAMIPEXOLE DIHYDROCHLORIDE:Influenza like illness:0.05), (PRAVASTATIN SODIUM:Influenza like illness:0.09), (QUETIAPINE:Influenza like illness:0.05), (RISEDRONATE SODIUM:Influenza like illness:0.14), (SACUBITRILVALSARTAN:Influenza like illness:0.27), (TERIFLUNOMIDE:Influenza like illness:0.39), (THALIDOMIDE:Influenza like illness:0.03), (TIZANIDINE HYDROCHLORIDE:Influenza like illness:0.11), (AMIODARONE HYDROCHLORIDE:Infusion related reaction:0.01), (ANASTROZOLE:Infusion related reaction:0.22), (CALCIUMVITAMIN D:Infusion related reaction:0.06), (CARFILZOMIB:Infusion related reaction:0.16), (CERTOLIZUMAB PEGOL:Infusion related reaction:0.77), (CLONIDINE:Infusion related reaction:0), (DASATINIB:Infusion related reaction:0.17), (DICLOFENAC:Infusion related reaction:0.27), (DOXYCYCLINE:Infusion related reaction:0.11), (ERYTHROPOIETIN:Infusion related reaction:0.05), (ESZOPICLONE:Infusion related reaction:0.35), (EVEROLIMUS:Infusion related reaction:0.11), (FERROUS SULFATEFERROUS SULFATE, DRIED:Infusion related reaction:0.46), (INSULIN ASPART:Infusion related reaction:0.31), (IRON:Infusion related reaction:0.35), (LOVASTATIN:Infusion related reaction:0.16), (MELOXICAM:Infusion related reaction:0.32), (NILOTINIB:Infusion related reaction:0.54), (POTASSIUM:Infusion related reaction:0.31), (PRAVASTATIN:Infusion related reaction:0.03), (PYRAZINAMIDE:Infusion related reaction:0.62), (SACUBITRILVALSARTAN:Infusion related reaction:0.33), (THALIDOMIDE:Infusion related reaction:0.66), (VANCOMYCIN HYDROCHLORIDE:Infusion related reaction:0.16), (VERAPAMIL HYDROCHLORIDE:Infusion related reaction:0.23), (AMIODARONE HYDROCHLORIDE:Inhalation therapy:0.15), (AMPHETAMINE ASPARTATEAMPHETAMINE SULFATEDEXTROAMPHETAMINE SACCHARATEDEXTROAMPHETAMINE SULFATE:Inhalation therapy:0.51),(ARMODAFINIL:Inhalation therapy:0.21), (CALCIUM CARBONATECHOLECALCIFEROL:Inhalation therapy:0.47), (CARVEDILOL PHOSPHATE:Inhalation therapy:0), (CEPHALEXIN:Inhalation therapy:0.11), (CERTOLIZUMAB PEGOL:Inhalation therapy:0.36), (CHOLESTYRAMINE:Inhalation therapy:0.08), (CLINDAMYCINCLINDAMYCIN PHOSPHATE:Inhalation therapy:0.5), (DULOXETINE:Inhalation therapy:0.11), (ELETRIPTAN HYDROBROMIDE:Inhalation therapy:0.02), (ENOXAPARIN:Inhalation therapy:0.06), (EVOLOCUMAB:Inhalation therapy:0.69), (FLUTICASONE FUROATE:Inhalation therapy:0.02), (FORMOTEROL FUMARATEMOMETASONE FUROATE:Inhalation therapy:0.5), (HYDROXYZINE PAMOATE:Inhalation therapy:0.11), (IBRUTINIB:Inhalation therapy:0.53), (ILOPROST:Inhalation therapy:0.45), (INDOMETHACIN:Inhalation therapy:0.13), (LABETALOLLABETALOL HYDROCHLORIDE:Inhalation therapy:0.06), (LIDOCAINEPRILOCAINE:Inhalation therapy:0.06), (MEPOLIZUMAB:Inhalation therapy:0.41), (METHYLPREDNISOLONE SODIUM SUCCINATE:Inhalation therapy:0.12), (NEBIVOLOL:Inhalation therapy:0.08), (OLANZAPINE:Inhalation therapy:0.14), (PANTOPRAZOLE MAGNESIUM:Inhalation therapy:0.62), (POLYETHYLENE GLYCOLS:Inhalation therapy:0.43), (PRAMIPEXOLE DIHYDROCHLORIDE:Inhalation therapy:0.24), (PROMETHAZINEPROMETHAZINE HYDROCHLORIDE:Inhalation therapy:0.26), (SODIUM OXYBATE:Inhalation therapy:0.39), (VITAMIN B:Inhalation therapy:0.17), (LOVASTATIN:Initial insomnia:0.08), (OXCARBAZEPINE:Initial insomnia:0.42), (TERIFLUNOMIDE:Initial insomnia:0.14), (VARENICLINE TARTRATE:Initial insomnia:0.3), (WARFARIN:Initial insomnia:0.2), (CALCIUM CARBONATECHOLECALCIFEROL:Injury:0.13), (CERTOLIZUMAB PEGOL:Injury:0.35), (CITALOPRAM HYDROBROMIDE:Injury:0.52), (DEXLANSOPRAZOLE:Injury:0.53), (DIGOXIN:Injury:0.22), (DILTIAZEM HYDROCHLORIDE:Injury:0.06), (DIVALPROEX SODIUM:Injury:0.75), (ESOMEPRAZOLE MAGNESIUM:Injury:0.53), (EVOLOCUMAB:Injury:0.5), (FERROUS SULFATEFERROUS SULFATE, DRIED:Injury:0.11), (FLUTICASONE PROPIONATE:Injury:0.11), (IBRUTINIB:Injury:0.21), (LOVASTATIN:Injury:0.4), (METHYLP REDNISOLONE SODIUM SUCCINATE:Injury:0.08), (PANTOPRAZOLE:Injury:0.17), (QUETIAPINE:Injury:0.02), (VAR ENICLINE TARTRATE:Injury:0.26), (.BETA.-ACETYLDIGOXIN:Insomnia:0.4), (.BETA.-CAROTENE:Insomnia:0.38), (.BETA.-HYDROXYISOVALERIC ACIDARGININEGLUTAMINE:Insomnia:0.74), (ABACAVIRLAMIVUDINEZIDOVUDIN E:Insomnia:0.16), (ACETAMINOPHENASPIRIN:Insomnia:0.12), (ACETAMINOPHENASPI RINCAFFEINECODEINE PHOSPHATE:Insomnia:0.02), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Insomnia:1.49), (ACETAMINOPHENBUCLIZINE HYDROCHLORIDECODEINE:Insomnia:0.33), (ACETAMINOPHENBUTYLSCOPOLAMIN E BROMIDE:Insomnia:0.33), (ACETAMINOPHENCAFFEINECARISOPRODOLDICLOFEN AC:Insomnia:0.98), (ACETAMINOPHENCAFFEINEPROPOXYPHENE:Insomnia:0.41), (AC ETAMINOPHENCHLORPHENIRAMINE MALEATEDEXTROMETHORPHAN HYDROBROMIDEPHENYLEPHRINE HYDROCHLORIDE OR ACETAMINOPHENDEXTROMETHORPHAN HYDROBROMIDEDOXYLAMINE SUCCINATEPHENYLEPHRINE HYDROCHLORIDE OR HOMEOPATHICS:Insomnia:0.16), (ACETAMINOPHENCHLORPHENIRAMINE MALEATEPHENYLEPHRINE HYDROCHLORIDE:Insomnia:0.31), (ACETAMINOPHENCHLORPHENIRAMINE MALEATEPSEUDOEPHEDRINE HYDROCHLORIDE:Insomnia:1.02), (ACETAMINOPHENDEXTROMETHORPHAN HYDROBROMIDEPHENYLEPHRINE HYDROCHLORIDE:Insomnia:0.11), (ACETAMINOPHENDICHLORALPHENAZONEISO METHEPTENE HYDROCHLORIDE:Insomnia:0.28), (ACETAMINOPHENGUAIFENESINPHENYLEPHRIN E HYDROCHLORIDE:Insomnia:0.17), (ACETAMINOPHENMETHOCARBAMOL:Insomnia:1. 64), (ACETOHYDROXAMIC ACID:Insomnia:0.06), (ACETYLCARNITINE:Insomnia:0.63), (ACETYLCYSTEINEBENZAL KONIUM CHLORIDETUAMINOHEPTANE SULFATE:Insomnia:1.13), (ACRIVASTINE:Insomnia:0.13), (ACRIVASTINEPSEUDOEPHE DRINE HYDROCHLORIDE:Insomnia:1.18), (AJMALINE:Insomnia:1.05), (ALBIGLUTIDE:Insomnia:0 0.34), (ALBUTEROLGUAIFENESIN:Insomnia:0.87), (ALFALFA:Insomnia:0.74), (ALGELDRA TE:Insomnia:0.1), (ALGINIC ACID:Insomnia:0.18), (ALISKIREN HEMIFUMARATEHYDROCHLOROTHIAZIDE:Insomnia:0.79), (ALISKIREN HEMIFUMARATEVALSARTAN:Insomnia:0.61), (ALPHA-TOCOPHEROLASCORBIC ACIDCHOLECALCIFEROLCOPPERCYANOCOBALAMINFOLIC ACIDLUTEINOMEGA-3 FATTY ACIDSPYRIDOXINE HYDROCHLORIDETAURINEVITAMIN A PALMITATEZEAXANTHINZINC PICOLINATE:Insomnia:0.11), (ALUMINUM ACETATE:Insomnia:0.02), (ALUMINUM CHLORIDE:Insomnia:0.44), (ALVERINE CITRATE:Insomnia:0.08), (AMILORIDE HYDROCHLORIDEFUROSEMIDE:Insomnia:1.15), (AMINOSALICYLIC ACID:Insomnia:1.12), (AMITRIPTYLINEPERPHENAZINE:Insomnia:0.38), (AMLODIPINE BESYLATEINDAPAMIDEPERINDOPRIL ARGININE:Insomnia:0.08), (AMLODIPINELOSARTAN:Insomnia:0.49), (AMOROLFINE:Ins omnia:0.01), (AMPHETAMINE ASPARTATE MONOHYDRATEAMPHETAMINE SULFATEDEXTROAMPHETAMINE SACCHARATEDEXTROAMPHETAMINE SULFATE:Insomnia:0.02), (AMPHETAMINE ASPARTATEDEXTROAMPHETAMINE SACCHARATE:Insomnia:0.13), (AMPHOTERICIN BDIMYRISTOYLPHOSPHATIDYLCHOLINE, DL-DIMYRISTOYLPHOSPHATIDYLGLYCEROL, DL-:Insomnia:0.32), (ANHYDROUS CITRIC ACIDMAGNESIUM OXIDESODIUM PICOSULFATE:Insomnia:0.24), (ANTIHEMOPHILIC FACTOR, HUMAN RECOMBINANT RESIDUES 743-1636 DELETED:Insomnia:0.2), (ARCTOSTAPHYLOS UVA-URSI LEAFURTICA DIOICA:Insomnia:0.48), (ARSENIC TRIOXIDE:Insomnia:0.81), (ASCORBIC ACID/CHROMIUM/SELENIUM/VANADYL SULFATE:Insomnia:1.3), (ASCORBIC ACIDCHONDROITIN SULFATE (BOVINE)GLUCOSAMINE SULFATEMANGANESE:Insomnia:0.31), (ASCORBIC ACIDFERROUS FUMARATEFOLIC ACID:Insomnia:0.1), (ASCORBIC ACIDVITAMIN B COMPLEX:Insomnia:0.68), (ASPIRINCAFFEINESALICYLAMIDE:Insomnia:0.72), (ATAZA NAVIRRITONAVIR:Insomnia:0.18), (ATRACURIUM:Insomnia:0.78), (ATRACURIUM BESYLATE:Insomnia:0.48), (ATROPINE SULFATEDIFENOXIN HYDROCHLORIDE:Insomnia:0.15), (ATROPINE SULFATEDIPHENOXYLATE:Insomnia:0.51), (AVANAFIL:Insomnia:0.14), (AVOBENZONE HOMOSALATEOCTISALATE:Insomnia:0.76), (AZITHROMYCIN:Insomnia:0.69), (AZITHR OMYCIN OR METHOTREXATE SODIUM:Insomnia:2.29), (BACILLUS CALMETTE-GUERIN SUBSTRAIN CONNAUGHT LIVE ANTIGEN:Insomnia:0.01), (BACITRACIN ZINCNEOMYCIN SULFATEPOLYMYXIN B SULFATE:Insomnia:0.38), (BACITRACINNEOMYCIN SULFATEPOLYMYXIN B SULFATE:Insomnia:0.07), (BACLOFENCYCLOBENZAPRINEDICLOFENACGABAPENT INTETRACAINE:Insomnia:0.64), (BALSAM PERU OILCASTOR OILTRYPSIN:Insomnia:0.37), (BARNIDIPINE HYDROCHLORIDE:Insomnia:0.02), (BECLOMETHASONE DIPROPIONATE MONOHYDRATE:Insomnia:0.56), (BENPERIDOL:Insomnia:0.66), (BENRALIZUMAB:Insom nia:0.13), (BENZOCAINERESORCINOL:Insomnia:0.17), (BEPOTASTINEBEPOTASTINE BESILATE:Insomnia:0.11), (BEPRIDIL HYDROCHLORIDE:Insomnia:0.31), (BETAINE:Insomnia:0.26), (BETAMETHASONE DIPROPIONATECALCIPOTRIENE HYDRATE:Insomnia:1.07), (BETAMETHASONE DIPROPIONATECLOTRIMAZOLEGENTAMICIN SULFATE:Insomnia:0.1), (BETAMETHASONE VALERATEFUSIDIC ACID:Insomnia:0.16), (BEXAROTENE:Insomnia:0.75), (BIOTINCALCIUM PANTOTHENATECHOLINE BITARTRATECYANOCOBALAMINFOLIC ACIDINOSITOLNIACINAMIDEPYRIDOXINERIBOFLAVINTHIAMINE:Insomnia:0.08), (BISMUTH SUBSALICYLATEGLYCERINPROPYLENE GLYCOL:Insomnia:0), (BLESELUMAB:Insomnia:0.04), (BORNAPRINE HYDROCHLORIDE:Insomnia:0.33), (BORON:Insomnia:0.57), (BOTULISM ANTITOXIN:Insomnia:0.09), (BRIGATINIB:Insomnia:0.09), (BRODALUMAB:Insomnia:0.45), (BUDESONIDEINDACATEROL MALEATE:Insomnia:0.18), (BUFORMIN:Insomnia:1.33), (BUPROPION HYDROBROMIDE:Insomnia:0.65), (CAFFEINEISOMETHEPTENEMETHIMAZOLE:Insom nia:0.52), (CAFFEINEPHENOBARBITAL:Insomnia:0.01), (CAJUPUT OILEUCALYPTUS OILPEPPERMINT OIL:Insomnia:1.22), (CALCIUM ACETATEMAGNESIUM ACETATEPOTASSIUM ACETATESODIUM ACETATESODIUM CHLORIDE:Insomnia:0.96), (CALCIUM CHLORIDEMAGNESIUM CHLORIDEPOTASSIUM CHLORIDESODIUM ACETATESODIUM CHLORIDE:Insomnia:0.27), (CALCIUM GLYCEROPHOSPHATE:Insomnia:0.91), (CALCIUM PHOSPHATE:Insomnia:0.39), (CALCIUM PHOSPHATE, DIBASIC, ANHYDROUSFERROUS SUCCINATEMAGNESIUM HYDROXIDE:Insomnia:0.14), (CALCIUMERGOCALCIFEROL:Insomnia:0.52), (CAMPHOR (NATURAL)MENTHOLPRAMOXINE HYDROCHLORIDE:Insomnia:0.95), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:Insomnia:0.37), (CANNABIDIOL:Insomnia:1.37), (CANNABIS SATIVA SEED OIL:Insomnia:0.07), (CANNABIS SATIVA SUBSP. SATIVA FLOWERING TOP:Insomnia:1.26), (CAPSICUM:Insomnia:0.32), (CAPTOPRILHYDROCHLOROTHIAZIDE:Insomnia:0.01), (CARBASPIRIN:Insomnia:0.71), (CARBOMER 934\GLYCERINMETHYLPARABENPROPYLPARABENSODIUM HYDROXIDE:Insomnia:0.18), (CARBOXYMETHYLCELLULOSE SODIUMGELATINPECTIN:Insomnia:0), (CARTEOLOL:Insomnia:0.54), (CARVEDILOL PHOSPHATE:Insomnia:3.21), (CEFOPERAZONE:Insomnia:0.15), (CEFOTAXIMECEFOTAX IME SODIUM:Insomnia:1.76), (CEFPIROME SULFATE:Insomnia:1.45), (CEFPROZIL:Insomnia:2.18), (CELERY SEED:Insomnia:0.45), (CEPHALOSPORIN C:Insomnia:0.08), (CERIVASTATIN SODIUM:Insomnia:1.28), (CERTOLIZUMAB:Insomnia:0.24), (CHAMOMILE:Insomnia:0.62), (CHLOPHEDIANOL HYDROCHLORIDEDEXBROMPHENIRAMINE MALEATEPHENYLEPHRINE HYDROCHLORIDE:Insomnia:0.1), (CHLORDIAZEPOXIDECLIDINIUM:Insomnia:0.35), (C HLORHEXIDINE HYDROCHLORIDENEOMYCIN SULFATE:Insomnia:0.05), (CHLORIDE ION:Insomnia:0.11), (CHLORMADINONE ACETATEETHINYL ESTRADIOL:Insomnia:0.78), (CHLORMADINONE ACETATEMESTRANOL:Insomnia:0.35), (CHLORPHENIRAMINE MALEATEHYDROCODONE BITARTRATEPSEUDOEPHEDRINE HYDROCHLORIDE:Insomnia:0.29), (CHOLECALCIFEROLGENISTEINZINC GLYCINATE:Insomnia:0.22), (CHOLIC ACID:Insomnia:0.13), (CHOLINETRAZODONE HYDROCHLORIDE:Insomnia:0.22), (CICLOPIROX OLAMINE:Insomnia:1.6), (CITICOLINE SODIUM:Insomnia:0.72), (CITRIC ACID MONOHYDRATE:Insomnia:0.3), (CITRIC ACID MONOHYDRATETRISODIUM CITRATE DIHYDRATE:Insomnia:0.24), (CLENBUTEROL HYDROCHLORIDE:Insomnia:2.36), (CLOBETASONE:Insomnia:0.13), (CLOBETASONE BUTYRATENYSTATINOXYTETRACYCLINE CALCIUM:Insomnia:0.14), (CLOPENTHIXOL HYDROCHLORIDE:Insomnia:0.06), (CLOSTRIDIUM TETANI TOXOID ANTIGEN (FORMALDEHYDE INACTIVATED)CORYNEBACTERIUM DIPHTHERIAE TOXOID ANTIGEN (FORMALDEHYDE INACTIVATED):Insomnia:0.11), (CLOSTRIDIUM TETANICLOSTRIDIUM TETANI TOXOID ANTIGEN (FORMALDEHYDE INACTIVATED)CORYNEBACTERIUM DIPHTHERIAECORYNEBACTERIUM DIPHTHERIAE TOXOID ANTIGEN (FORMALDEHYDE INACTIVATED):Insomnia:0.44), (CLOXACILLIN SODIUM:Insomnia:1.06), (COBALAMIN:Insomnia:1.25), (COPPER OR LEVONORGESTREL:Insomnia:0.5), (CREATINE MONOHYDRATE:Insomnia:1.09), (CREATININE:Insomnia:0.28), (CROTALIDAE POLYVALENT IMMUNE FAB (OVINE):Insomnia:0.3), (CYANOCOBALAMINPYRIDOXINE HYDROCHLORIDERIBOFLAVINSODIUM PANTOTHENATETHIAMINE HYDROCHLORIDEVITAMIN B COMPLEX:Insomnia:0.98), (CYANOCOBALAMINPYRIDOXINE HYDROCHLORIDETHIAMINE DISULFIDE:Insomnia:1.53), (CYCLIZINE HYDROCHLORIDE:Insomnia:0.45), (CYCLOPHOSPHAMIDEDOXORUBICINPREDNISO NERITUXIMABVINCRISTINE:Insomnia:0.39), (CYPROTERONE:Insomnia:0.93), (CYSTEI NEGLYCINEGLYCYRRHIZIN:Insomnia:0.43), (CYTARABINE OCFOSFATE:Insomnia:1.07), (DARUNAVIRRITONAVIR:Insomnia:0.11), (DASABUVIR SODIUM MONOHYDRATE:Insomnia:0.06), (DEFIBROTIDE (BOVINE):Insomnia:0.37), (DEOXYRIBONUCLIEC ACID:Insomnia:0.27), (DEXAMETHASONEFRAMYCETIN SULFATEGRAMICIDIN:Insomnia:0.22), (DEXAMETHASONENEOMYCIN:Insomnia:0.04), (DEXMEDETOMIDINE:Insomnia:0.48), (DEXRAZOXANE HYDROCHLORIDE:Insomnia:0.07), (DEXTROMETHORPHAN HYDROBROMIDEGUAIFENESINPSEUDOEPHEDRINE HYDROCHLORIDE:Insomnia:0.45), (DEXTROMETHORPHANDEXTROMETHORPHAN HYDROBROMIDEGUAIFENESIN:Insomnia:0.81), (DEXTROSE MONOHYDRATESODIUM CHLORIDE:Insomnia:0.48), (DIBUCAINE:Insomnia:0.04), (DICHLORPHENAMIDE:Insomnia:0.36), (DICLOXACILLIN SODIUM:Insomnia:0.27), (DIETHYLPROPION:Insomnia:0.33), (DIFLUNISAL:Insomnia:1.88), (DIGITALIS:Insomnia:0.38), (DILAZEP DIHYDROCHLORIDE:Insomnia:0.52), (DIMETHYL SULFOXIDE:Insomnia:0.67), (DIPHENHYDRAMINE HYDROCHLORIDEPHENYLEPHRINE HYDROCHLORIDE:Insomnia:0.42), (DOCONEXENTICOSAPENT:Insomnia:0.02), (DOXIFL URIDINE:Insomnia:0.12), (DOXYCYCLINE HYDROCHLORIDE:Insomnia:1.09), (ECHINACEA PURPUREA:Insomnia:0.33), (ELOSULFASE ALFA:Insomnia:0.54), (ENALAPRIL MALEATELERCANIDIPINE HYDROCHLORIDE:Insomnia:1.07), (EPHEDRINE HYDROCHLORIDEGUAIFENESIN:Insomnia:1.16), (ERYTHROMYCINSULFISOXAZOLE:Insomnia:1.29), (ESCHERICHIA COLI:Insomnia:0.16), (ESLICARBAZEPINE:Insomnia:0.02), (ESMOLOL HYDROCHLORIDE:Insomnia:1.47), (ESTRADIOL BENZOATE:Insomnia:1.66), (ESTRADIOL HEMIHYDRATENORETHINDRONE ACETATE:Insomnia:0.02), (ESTRADIOLESTRIOL:Insomnia:0.45), (ESTRAMUSTINE:Insom nia:0.51), (ETEPLIRSEN:Insomnia:0.09), (ETHINYL ESTRADIOL:Insomnia:0.84), (ETHINYL ESTRADIOLETHYNODIOL:Insomnia:0.37), (ETHINYL ESTRADIOLETHYNODIOL DIACETATE:Insomnia:1.23), (ETHINYL ESTRADIOLLEVONORGESTREL ACETATE:Insomnia:0.98), (ETHIODIZED OIL:Insomnia:0.17), (FELODIPINEMETOPROLOL SUCCINATE:Insomnia:0.6), (FENOPROFEN:Insomnia:0.32), (FENOPROFEN CALCIUM:Insomnia:0.94), (FIMASARTAN:Insomnia:0.63), (FLUNARIZINE:Insomnia:0.86), (FLUOROURACILIRINOTECANLEUCOVORIN:Insomnia:0.39), (FLUPENTIXOL DECANOATE:Insomnia:1.34), (FLUPENTIXOL DIHYDROCHLORIDE:Insomnia:1.11), (FOSBRETABULIN TROMETHAMINE:Insomnia:0.11), (FUSIDATE SODIUM:Insomnia:0.53), (GADOPENTETATE DIMEGLUMINE:Insomnia:0.69), (GADOTERATE MEGLUMINE:Insomnia:0.3), (GADOVERSETAMIDE:Insomnia:0.57), (GENISTEIN:Insomnia: 0.13), (GENTIAN VIOLET:Insomnia:0.66), (GINKGO BILOBA LEAF EXTRACT:Insomnia:0.29), (GINSENG EXTRACT:Insomnia:0.04), (GLIQUIDONE:Insomnia:0.22), (GLUCONATE:Insomnia:0.5), (GL UCOSAMINE HYDROCHLORIDE:Insomnia:0.38), (GLYCOPYRROLATEINDACATEROL MALEATE:Insomnia:2.08), (GREEN TEA LEAF:Insomnia:0.65), (GUAIFENESINPHENYLEPHRINE HYDROCHLORIDE:Insomnia:1.06), (GUSPERIMUS TRIHYDROCHLORIDE:Insomnia:0.29), (HERBALSVITIS VINIFERA SEED:Insomnia:0.29), (HIGH DENSITY POLYETHYLENE:Insomnia:1.08), (HUMAN COLOSTRUM:Insomnia:0.1), (HYDROCHLORIC ACID:Insomnia:0.32), (HYDROCHLOROTHIAZIDEMETOPROLOL:Insomnia:0.02), (HYDR OCHLOROTHIAZIDEPROPRANOLOL HYDROCHLORIDE:Insomnia:0.05), (HYDROCORTISONE HEMISUCCINATE:Insomnia:0.5), (HYDROCORTISONEPRAMOXINE:Insomnia:0.08), (HY DROXOCOBALAMIN ACETATE:Insomnia:0.89), (INDISETRON HYDROCHLORIDE:Insomnia:0.09), (INOSINE PRANOBEX:Insomnia:0.03), (INSULIN PORKINSULIN PURIFIED PORK:Insomnia:1.26), (INTERFERON GAMMA-1B:Insomnia:2.4), (INULIN:Insomnia:0.01), (INVESTIGATIONAL BIOSIMILARS:Insomnia:0.17), (IODINEPOTASSIUM IODIDE:Insomnia:0.72), (IPRAGLIFLOZIN:Insomnia:0.68), (IRBESARTANTRICHLORMET HIAZIDE:Insomnia:0.6), (IRONVITAMIN B:Insomnia:0.06), (ISAVUCONAZOLE:Insomnia:0.32), (ISOCARBOXAZID:Insomnia:0.09), (I SOPROPYL ALCOHOL:Insomnia:0.04), (IXAZOMIB CITRATE:Insomnia:0.15), (LACTIC ACID:Insomnia:0.18), (LACTOBACILLUS ACIDOPHILUSLACTOBACILLUS RHAMNOSUS:Insomnia:0.88), (LACTOSE:Insomnia:0.43), (LANINAMIVIR:Insomnia:0.31), (LANOLINMINERAL OILPETROLATUM:Insomnia:0.79), (LANOLINPETROLATUM:Insomnia:0.29), (LARONID ASE:Insomnia:0.65), (LATANOPROSTTIMOLOL:Insomnia:0.89), (LAVENDER OIL:Insomnia:0.35), (LECITHINSODIUM BICARBONATESODIUM PHOSPHATE, MONOBASIC:Insomnia:0.88), (LETROZOLERIBOCICLIB:Insomnia:0.39), (LEVAMLODIPI NE BESYLATE:Insomnia:0.58), (LEVOBUNOLOL:Insomnia:0.63), (LEVOBUNOLOL HYDROCHLORIDE:Insomnia:0.45), (LEVOBUPIVACAINE:Insomnia:0.16), (LEVOMEFOLA TE CALCIUMMETHYLCOBALAMINPYRIDOXAL 5-PHOSPHATE:Insomnia:0.22), (LEVOMEPROMAZINE HYDROCHLORIDELEVOMEPROMAZINE MALEATE:Insomnia:0.84), (LEVORPHANOL TARTRATE:Insomnia:0.13), (LEVOSIMENDAN:Insomnia:0.57), (LEVOTHYROXINE SODIUMPOTASSIUM IODIDE:Insomnia:1.66), (LOFEPRAMINE:Insomnia:0.56), (LOPINAVIR:Insomnia:0.28), (LUM ACAFTOR:Insomnia:1.17), (LUTETIUM LU-177:Insomnia:1.32), (LYCOPENE:Insomnia:0.37), (LYSERGIDE:Insomnia:0.22), (MAGNESIU M GLUCEPTATE:Insomnia:0.23), (MANGANESEMANGANESE CHLORIDE:Insomnia:0.43), (MECLIZINE MONOHYDROCHLORIDENIACIN:Insomnia:0.99), (MELPERONE HYDROCHLORIDE:Insomnia:0.1), (MEPENZOLATE BROMIDE:Insomnia:1.92), (MEPREDNISONE:Insomnia:0.49), (MEPROBAMATE:Insomnia:0. 18), (MESTRANOLNORETHINDRONE:Insomnia:1.18), (METAPROTERENOL SULFATE:Insomnia:0.3), (METFORMIN HYDROCHLORIDEROSIGLITAZONE MALEATE:Insomnia:0.04), (METFORMIN HYDROCHLORIDESAXAGLIPTIN:Insomnia:1.28), (METFORMINSITAGLIPTIN:Insomnia:1.11), (METHAZOLAMIDE:Insomnia:0.05), (METHENAMINE MANDELATE:Insomnia:1.86), (METHIONINE:Insomnia:0.13), (METHSUXIMIDE:Insomnia:0 0.86), (METHYLENE BLUE:Insomnia:0.81), (METHYLENEDIOXYMETHAMPHETAMINE:Insomnia:0.44), (METH YLPREDNISOLONE OR METHYLPREDNISOLONE ACETATE:Insomnia:0.42), (METRELEPTIN:Insomnia:1. 8), (METRONIDAZOLE BENZOATE:Insomnia:0.09), (METYROSINE:Insomnia:0.59), (MIDOSTAURIN:Insomnia:0.09), (MIGLUSTAT:Insomnia:2.14), (MILRINONE LACTATE:Insomnia:0.01), (MINERALSPROBIOTICS NOSVITAMINS:Insomnia:0.31), (MOEXIPRIL HYDROCHLORIDE:Insomnia:2.06), (NAFTIFINE:Insomnia:0.1), (NALBUPHINE HYDROCHLORIDE:Insomnia:1.29), (NATURAL LATEX RUBBER:Insomnia:0.15), (NEISSERIA MENINGITIDIS GROUP A CAPSULAR POLYSACCHARIDE DIPHTHERIA TOXOID CONJUGATE ANTIGENNEISSERIA MENINGITIDIS GROUP C CAPSULAR POLYSACCHARIDE DIPHTHERIA TOXOID CONJUGATE ANTIGENNEISSERIA MENINGITIDIS GROUP W-135 CAPSULAR POLYSACCHARIDE DIPHTHERIA TOXOID CONJUGATE ANTIGENNEISSERIA MENINGITIDIS:Insomnia:0.43), (NELFINAVIR:Insomnia:0.16), (NEOSTIGMINE BROMIDE:Insomnia:0.18), (NEOSTIGMINE METHYLSULFATE:Insomnia:0.39), (NIACINAMIDEPYRIDOXINERIBOFLAVINTHIAMI NE:Insomnia:0.21), (NIAOULI OILQUININE BENZOATETHIAMINE HYDROCHLORIDE:Insomnia:1.37), (NIFUROXAZIDE:Insomnia:0.41), (NILUTAMIDE:Inso mnia:0.01), (NIMUSTINE:Insomnia:0.72), (NOMEGESTROL ACETATE:Insomnia:1.01), (NUSINERSEN:Insomnia:1.3), (OBETICHOLIC ACID:Insomnia:0.29), (OK-432:Insomnia:1.13), (OPICAPONE:Insomnia:0.96), (ORPHENADRINE HYDROCHLORIDE:Insomnia:0.43), (OSTREA EDULIS SHELL:Insomnia:0.26), (OTILONIUM BROMIDE:Insomnia:0.65), (OXANDROLONE:Insomnia:1.02), (OXETORONE:Insomnia:0.16), (OXICONAZOLE NITRATE:Insomnia:0.11), (OXYTETRACYCLINE:Insomnia:0.04), (OZAGREL:Insomnia:0.47), (PAMABROM:Insomnia:0.05), (PARSLEY:Insomnia:0.23), (PASCOPYRUM SMITHII POLLEN:Insomnia:0.16), (PEGINTERFERON LAMBDA-1A:Insomnia:0.13), (PEGVISOMANT:Insomnia:0.96), (PENICILLAMINE:Insomnia:1.05), (PEN TAERYTHRITOL TETRANITRATE:Insomnia:1.12), (PENTOBARBITAL:Insomnia:0.15), (PENTOSTATIN:Inso mnia:2.09), (PENTOXYVERINE CITRATE:Insomnia:0.55), (PEPSIN:Insomnia:0.57), (PERAZINE:Insomnia:0.61), (PERMETHRI N:Insomnia:0.47), (PHENAZOPYRIDINE HYDROCHLORIDE:Insomnia:1.72), (PHENOXYMETHYLPENICILLIN CALCIUM:Insomnia:0.29), (PHENYLBUTAZONE:Insomnia:0.09), (PHENYLEPHRINE HYDROCHLORIDEPROMETHAZINE HYDROCHLORIDE:Insomnia:0.97), (PHENYLEPHRINE HYDROCHLORIDEWITCH HAZEL:Insomnia:0.86), (PHENYLPROPANOLAMINE:Insomnia:0.12), (PHOSPHOLIPID:Inso mnia:0.22), (PILSICAINIDE:Insomnia:0.36), (PIMAVANSERIN:Insomnia:0.21), (PIPOTIAZIN E:Insomnia:1.02), (PIPOTIAZINE PALMITATE:Insomnia:0.74), (PIRENZEPINE HYDROCHLORIDE:Insomnia:0.18), (PIRETANIDE:Insomnia:0.42), (PIRIBEDIL:Insomnia:1.9), (PIRITRAMIDE:Insomnia:1.49), (PLASMA PROTEIN FRACTION (HUMAN):Insomnia:0.29), (PODOFILOX:Insomnia:0.16), (POLYETHYLENE GLYCOL 400:Insomnia:1.03), (POMEGRANATE:Insomnia:0.04), (POTASSIUM BICARBONATEPOTASSIUM CITRATE:Insomnia:0.1), (POTASSIUM BITARTRATE:Insomnia:0.06), (POTASSIUM BROMIDE:Insomnia:1.07), (POTASSIUM CHLORIDESODIUM CHLORIDE:Insomnia:0.17), (POTASSIUM CITRATESODIUM CITRATEZINC:Insomnia:0.36), (POTASSIUM PHOSPHATE, MONOBASIC:Insomnia:1.13), (PRAMOXINE HYDROCHLORIDE:Insomnia:1.48), (PREDNISOLONE ACETATESULFACETAMIDE SODIUM:Insomnia:0.81), (PREDNISOLONE SODIUM METAZOATE:Insomnia:0.91), (PROCARBAZINE:Insomnia:0.2), (PROCARBAZINE HYDROCHLORIDE:Insomnia:0.13), (PROPANTHELINE:Insomnia:0.16), (PROPARACAINE:I nsomnia:0.11), (PROPOLIS WAX:Insomnia:0.01), (PROPOXYPHENE:Insomnia:0.32), (PROTRIPTYLINE:Insomnia:0.32), (PROTRIPTYLINE HYDROCHLORIDE:Insomnia:2.8), (PSORALEN:Insomnia:0.39), (PYGEUM:Insomnia:0.52), (QUINIDINE GLUCONATE:Insomnia:0.54), (QUININE BISULFATE:Insomnia:0.14), (RADIUM:Insomnia:0.17), (RANIMUSTINE:Insomnia:0.94), (RED YEAST:Insomnia:0.24), (RESLIZUMAB:Insomnia:0.19), (RILPIVIRINE:Insomnia:0.33), (ROYAL JELLY:Insomnia:0.06), (RUPATADINE FUMARATE:Insomnia:1.1), (SAFINAMIDE:Insomnia:1.25), (SAPROPTERIN DIHYDROCHLORIDE:Insomnia:0.76), (SCOPOLAMINE HYDROBROMIDE:Insomnia:0.95), (SENNOSIDE A:Insomnia:1.28), (SENNOSIDE B:Insomnia:1.57), (SEROTONIN:Insomnia:0.13), (SERTACONAZOLE NITRATE:Insomnia:0.41), (SERTINDOLE:Insomnia:1.8), (SESAME OIL:Insomnia:0.07), (SHARK CARTILAGESHARK, UNSPECIFIED:Insomnia:0.49), (SIBUTRAMINE HYDROCHLORIDE:Insomnia:2.47), (SILTUXIMAB:Insomnia:0.1), (SIMEPREVIR SODIUM:Insomnia:0.04), (SODIUM FLUORIDEVITAMINS:Insomnia:0.61), (SODIUM HYPOCHLORITE:Insomnia:0.68), (SODIUM PHOSPHATE, MONOBASIC, ANHYDROUS:Insomnia:0.86), (SOLIFENACINTAMSULOSIN:Insomnia:0.25), (SOMATOST ATIN:Insomnia:0.32), (SOMATREM:Insomnia:0.55), (SOY ISOFLAVONES:Insomnia:0.06), (SPIRULINA:Insomnia:0.87), (STREPTOMYCIN SULFATE:Insomnia:1.31), (STREPTOMYCINSTREPTOMYCIN SULFATE:Insomnia:0.6), (STRONTIUM:Insomnia:0.17), (SUFENTANIL CITRATE:Insomnia:2.1), (SULBUTIAMINE:Insomnia:0.72), (SULFACETAMIDE SODIUM:Insomnia:0.87), (SUPROFEN:Insomnia:0.35), (TABALUMAB:Insomnia:1.05), (TALT IRELIN:Insomnia:0.67), (TAZOBACTAM SODIUM:Insomnia:0.82), (TECELEUKIN:Insomnia:0.93), (TEDIZOLID:Insomnia:0.13), (TEGA SEROD:Insomnia:0.69), (TELOTRISTAT ETHYL:Insomnia:1.35), (TEMOCAPRIL:Insomnia:0.94), (THIOCTIC ACID:Insomnia:0.95), (THIOGUANINE:Insomnia:0.23), (THIOPENTAL SODIUM:Insomnia:0.18), (THIOTHIXENE HYDROCHLORIDE:Insomnia:1.11), (THYROTROPIN ALFA:Insomnia:0.15), (TILIDINE HYDROCHLORIDE:Insomnia:0.75), (TIMOLOLTRAVOPROST:Insomnia:1.05), (TOBRAMY CIN SULFATE:Insomnia:0.1), (TOLBUTAMIDE:Insomnia:0.12), (TOLCAPONE:Insomnia:1.25), (T OLMETIN:Insomnia:0.34), (TOLMETIN SODIUM:Insomnia:0.25), (TOLPERISONE HYDROCHLORIDE:Insomnia:0.2), (TOREMIFENE:Insomnia:0.55), (TRIAMCINOLONE DIACETATE:Insomnia:0.27), (TRICAPRYLIN:Insomnia:0.18), (TRICLOSAN:Insomnia:0.37), (TRIMETHOBENZAMIDE:Insomnia:0.49), (TRIPTORELIN:Insomnia:0.55), (TRIPTORELIN PAMOATE:Insomnia:1. 82), (TROLAMINE:Insomnia:0.2), (TROPINE BENZYLATE:Insomnia:0.44), (UNDECYLENIC ACID:Insomnia:0.26), (URACIL:Insomnia:0.38), (VALRUBICIN:Insomnia:0.03), (VELAGLUC ERASE ALFA:Insomnia:0.78), (VITAMIN A PALMITATE:Insomnia:0.28), (VORAPAXAR SULFATE:Insomnia:0.17), (WHEY:Insomnia:0.52), (ZINC CITRATE:Insomnia:0.7), (ZIV-AFLIBERCEPT:Insomnia:0.79), (DEVICE:Insulin resistance:0.24), (EVOLOCUMAB:Insulin resistance:0.15), (BENAZEPRIL HYDROCHLORIDE:Insulin-requiring type 2 diabetes mellitus:0.07), (BUPROPION HYDROCHLORIDE:Insulin-requiring type 2 diabetes mellitus:0.01), (CALCITRIOL:Insulin-requiring type 2 diabetes mellitus:0.32), (CANAGLIFLOZINMETFORMIN HYDROCHLORIDE:Insulin-requiring type 2 diabetes mellitus:0.02), (CELECOXIB:Insulin-requiring type 2 diabetes mellitus:0.3), (DABIGATRAN:Insulin-requiring type 2 diabetes mellitus:0.07), (DASATINIB:Insulin-requiring type 2 diabetes mellitus:0.07), (DEXLANSOPRAZOLE:Insulin-requiring type 2 diabetes mellitus:0.23), (DILTIAZEM:Insulin-requiring type 2 diabetes mellitus:0.2), (EMPAGLIFLOZINLINAGLIPTIN:Insulin-requiring type 2 diabetes mellitus:0.28), (EMPAGLIFLOZINMETFORMIN HYDROCHLORIDE:Insulin-requiring type 2 diabetes mellitus:0.16), (ERGOCALCIFEROL:Insulin-requiring type 2 diabetes mellitus:0), (EVEROLIMUS:Insulin-requiring type 2 diabetes mellitus:0.17), (EVOLOCUMAB:Insulin-requiring type 2 diabetes mellitus:1.04), (FLECAINIDE:Insulin-requiring type 2 diabetes mellitus:0.28), (IBRUTINIB:Insulin-requiring type 2 diabetes mellitus:0.63), (INDAPAMIDEPERINDOPRIL:Insulin-requiring type 2 diabetes mellitus:0.25), (ISOSORBIDE:Insulin-requiring type 2 diabetes mellitus:0.2), (LEDIPASVIRSOFOSBUVIR:Insulin-requiring type 2 diabetes mellitus:0.01), (LENALIDOMIDE:Insulin-requiring type 2 diabetes mellitus:0.38), (LERCANIDIPINE HYDROCHLORIDE:Insulin-requiring type 2 diabetes mellitus:0.42), (LOVASTATIN:Insulin-requiring type 2 diabetes mellitus:0.99), (METHYLPREDNISOLONE SODIUM SUCCINATE:Insulin-requiring type 2 diabetes mellitus:0.1), (NILOTINIB:Insulin-requiring type 2 diabetes mellitus:0.15), (OXYGEN:Insulin-requiring type 2 diabetes mellitus:0.05), (RANOLAZINE:Insulin-requiring type 2 diabetes mellitus:0.84), (SOTALOL:Insulin-requiring type 2 diabetes mellitus:0.47), (LACOSAMIDE:Intellectual disability:0.04), (PHENYTOIN:Intellectual disability:0.07), (CLOBAZAM:Intentional overdose:0.14), (PHENYTOIN:Intentional self-injury:0.05), (ZONISAMIDE:Intentional self-injury:0.2), (BUMETANIDE:Intermittent claudication:0.04), (CALCIUM CARBONATECHOLECALCIFEROL:Intermittent claudication:0.06), (EVOLOCUMAB:Intermittent claudication:0.32), (ACARBOSE:Interstitial lung disease:0.48), (ACETAMINOPHENBUTALBITALCAFFEINE:Interstitial lung disease:0.06), (ACLIDINIUM BROMIDE:Interstitial lung disease:0.43), (AMISULPRIDE:Interstitial lung disease:0.12), (AMOXAPINE:Interstitial lung disease:0.07), (ANASTROZOLE:Interstitial lung disease:1.56), (APREPITANT:Interstitial lung disease:0.15), (BENAZEPRILBENAZEPRIL HYDROCHLORIDE:Interstitial lung disease:0.01), (BEPRIDIL:Interstitial lung disease:0.26), (BISACODYL OR DOCUSATE SODIUM:Interstitial lung disease:0.57), (BRINZOLAMIDE:Interstitial lung disease:0.25), (BUDESONIDEFORMOTEROL FUMARATE:Interstitial lung disease:0.34), (BUSPIRONE HYDROCHLORIDE:Interstitial lung disease:0.36), (CALCIUM ACETATE:Interstitial lung disease:0.34), (CALCIUM CARBONATECHOLECALCIFEROL:Interstitial lung disease:1.4), (CANAKINUMAB:Interstitial lung disease:0.32), (CARVEDILOL PHOSPHATE:Interstitial lung disease:0.45), (CEFAZOLIN SODIUM:Interstitial lung disease:0.1), (CHLORTHALIDONE:Interstitial lung disease:0.03), (CHOLESTYRAMINE:Interstitial lung disease:0.49), (CIMETIDINE:Interstitial lung disease:0.15), (CLINDAMYCIN:Interstitial lung disease:0.26), (CLINDAMYCINCLINDAMYCIN PHOSPHATE:Interstitial lung disease:1.08), (CORTISONEHYDROCORTISONE:Interstitial lung disease:0.5), (CYANOCOBALAMINDEXPANTHENOLNIACINAMIDEPYRIDOXINE HYDROCHLORIDERIBOFLAVIN 5′-PHOSPHATE SODIUMTHIAMINE HYDROCHLORIDEVITAMIN B COMPLEX:Interstitial lung disease:0.11), (DACLIZUMAB:Interstitial lung disease:0.27), (DAPAGLIFLOZIN PROPANEDIOL:Interstitial lung disease:0), (DASATINIB:Interstitial lung disease:1.45), (DESLORATADINE:Interstitial lung disease:0.52), (DEVICE:Interstitial lung disease:0.47), (DEXAMETHASONE ACETATE:Interstitial lung disease:0.81), (DICLOFENAC:Interstitial lung disease:0.82), (DILAZEP:Interstitial lung disease:0.07), (DIMENHYDRINATE:Interstitial lung disease:0.03), (DIVALPROEX SODIUM:Interstitial lung disease:0.44), (DOCUSATE:Interstitial lung disease:0.35), (DOFETILIDE:Interstitial lung disease:0.11), (DOXAZOSIN MESYLATE:Interstitial lung disease:0.79), (DRONABINOL:Interstitial lung disease:0.15), (ELETRIPTAN HYDROBROMIDE:Interstitial lung disease:0.22), (ENALAPRIL MALEATE:Interstitial lung disease:0.83), (EPINEPHRINE:Interstitial lung disease:0.8), (ETORICOXIB:Interstitial lung disease:0.16), (ETRETINATE:Interstitial lung disease:0.44), (EXEMESTANE:Interstitial lung disease:0.1), (FENOTEROL HYDROBROMIDEIPRATROPIUM BROMIDE:Interstitial lung disease:0.12), (FERROUS SULFATEFERROUS SULFATE, DRIED:Interstitial lung disease:1.43), (FESOTERODINE FUMARATE:Interstitial lung disease:0.61), (FLECAINIDE:Interstitial lung disease:0.87), (FLUTICASONE:Interstitial lung disease:0.86), (FORMOTEROL FUMARATEMOMETASONE FUROATE:Interstitial lung disease:0.31), (FULVESTRANT:Interstitial lung disease:0.36), (GEMCITABINEGEMCITABINE HYDROCHLORIDE:Interstitial lung disease:0.1), (GLYCOPYRROLATE:Interstitial lung disease:0.13), (HEPARIN CALCIUM:Interstitial lung disease:0.02), (HYDROCHLOROTHIAZIDELISINOPRIL:Interstitial lung disease:0.96), (HYDROMORPHONE:Interstitial lung disease:0.51), (HYDROXYZINE PAMOATE:Interstitial lung disease:0.36), (IDELALISIB:Interstitial lung disease:0.31), (IMIPRAMINE HYDROCHLORIDE:Interstitial lung disease:0.19), (INDAPAMIDEPERINDOPRIL:Interstitial lung disease:0.54), (INVESTIGATIONAL PRODUCT:Interstitial lung disease:0.14), (IRINOTECAN HYDROCHLORIDE:Interstitial lung disease:0.54), (LABETALOLLABETALOL HYDROCHLORIDE:Interstitial lung disease:0.32), (LERCANIDIPINE HYDROCHLORIDE:Interstitial lung disease:0.5), (LIDOCAINEPRILOCAINE:Interstitial lung disease:0.11), (LITHIUM CARBONATE:Interstitial lung disease:0.92), (MEPOLIZUMAB:Interstitial lung disease:0.3), (METHENAMINE HIPPURATE:Interstitial lung disease:0.1), (METHENOLONE ENANTHATE:Interstitial lung disease:0.17), (MEXILETINE HYDROCHLORIDE:Interstitial lung disease:0.04), (PANCRELIPASE AMYLASEPANCRELIPASE LIPASEPANCRELIPASE PROTEASE:Interstitial lung disease:0.03), (PANTOPRAZOLE MAGNESIUM:Interstitial lung disease:1.06), (PAROXETINEPAROXETINE HYDROCHLORIDE:Interstitial lung disease:0.09), (PAZOPANIB HYDROCHLORIDE:Interstitial lung disease:0.88), (PHENYTOIN SODIUM:Interstitial lung disease:0.03), (PIMOBENDAN:Interstitial lung disease:0.09), (PRAMIPEXOLE DIHYDROCHLORIDE:Interstitial lung disease:0.22), (RIVASTIGMINE TARTRATE:Interstitial lung disease:0), (ROFLUMILAST:Interstitial lung disease:0.7), (ROPINIROLE HYDROCHLORIDE:Interstitial lung disease:0.18), (ROSUVASTATIN:Interstitial lung disease:0.07), (SACUBITRILVALSARTAN:Interstitial lung disease:0.74), (SCOPOLAMINE:Interstitial lung disease:0.37), (SUPLATAST TOSILATE:Interstitial lung disease:0.18), (TERBUTALINE SULFATE:Interstitial lung disease:0.02), (TIMOLOL:Interstitial lung disease:0.56), (TIOTROPIUM:Interstitial lung disease:0.58), (TOPIRAMATE:Interstitial lung disease:0.95), (VARENICLINE TARTRATE:Interstitial lung disease:0.31), (WARFARIN POTASSIUM:Interstitial lung disease:0.43), (ZONISAMIDE:Interstitial lung disease:0.25), (.ALPHA.1-PROTEINASE INHIBITOR HUMAN:Intervertebral disc degeneration:0.09), (AZITHROMYCIN DIHYDRATE:Intervertebral disc degeneration:0.34), (BISOPROLOL:Intervertebral disc degeneration:0.01), (BUMETANIDE:Intervertebral disc degeneration:0.76), (CALCIUM ACETATE:Intervertebral disc degeneration:0.26), (CANAGLIFLOZINMETFORMIN HYDROCHLORIDE:Intervertebral disc degeneration:0.06), (CANDESARTAN:Intervertebral disc degeneration:0.63), (CANDESARTAN CILEXETILHYDROCHLOROTHIAZIDE:Intervertebral disc degeneration:0.27), (CARFILZOMIB:Intervertebral disc degeneration:0.02), (CHLORTHALIDONE:Intervertebral disc degeneration:0.19), (CLINDAMYCINCLINDAMYCIN PHOSPHATE:Intervertebral disc degeneration:0.72), (DALFAMPRIDINE:Intervertebral disc degeneration:0.34), (DASATINIB:Intervertebral disc degeneration:0.64), (DESLORATADINE:Intervertebral disc degeneration:0.14), (DIETARY SUPPLEMENT:Intervertebral disc degeneration:0.18), (DIMENHYDRINATE:Intervertebral disc degeneration:0.23), (DOFETILIDE:Intervertebral disc degeneration:0.33), (DOXAZOSIN MESYLATE:Intervertebral disc degeneration:0.12), (DOXAZOSINDOXAZOSIN MESYLATE:Intervertebral disc degeneration:0.01), (DRONABINOL:Intervertebral disc degeneration:0.13), (DUTASTERIDE:Intervertebral disc degeneration:0.24), (ENALAPRIL MALEATE:Intervertebral disc degeneration:0.36), (ENOXAPARIN:Intervertebral disc degeneration:0.66), (ESTROGENS:Intervertebral disc degeneration:0.05), (EVEROLIMUS:Intervertebral disc degeneration:0.1), (FISH OIL\OMEGA-3 FATTY ACIDS:Intervertebral disc degeneration:0.04), (FLUTICASONE FUROATE:Intervertebral disc degeneration:0.17), (HEPARIN SODIUM:Intervertebral disc degeneration:0.31), (HERBALSTURMERIC:Intervertebral disc degeneration:0.31), (HYDROCHLOROTHIAZIDEVALSARTAN:Intervertebral disc degeneration:0.31), (HYDROXYCHLOROQUINE DIPHOSPHATE:Intervertebral disc degeneration:0.45), (INDAPAMIDE:Intervertebral disc degeneration:0.03), (IPRATROPIUM:Intervertebral disc degeneration:0.42), (LABETALOLLABETALOL HYDROCHLORIDE:Intervertebral disc degeneration:0.53), (LEVOMEFOLATE CALCIUM:Intervertebral disc degeneration:0.01), (LIDOCAINEPRILOCAINE:Intervertebral disc degeneration:0.02), (LITHIUM CARBONATE:Intervertebral disc degeneration:0.06), (LOMITAPIDE MESYLATE:Intervertebral disc degeneration:0.16), (METHENAMINE HIPPURATE:Intervertebral disc degeneration:0.24), (METRONIDAZOLEMETRONIDAZOLE HYDROCHLORIDE:Intervertebral disc degeneration:0.41), (NILOTINIB:Intervertebral disc degeneration:0.31), (NITROFURANTOINNITROFURANTOIN MONOHYDRATE:Intervertebral disc degeneration:0.13), (NORETHINDRONE:Intervertebral disc degeneration:0.08), (OLANZAPINE:Intervertebral disc degeneration:0.36), (PANTOPRAZOLE MAGNESIUM:Intervertebral disc degeneration:0.81), (PEGINTERFERON BETA-1A:Intervertebral disc degeneration:0.34), (PHENOBARBITAL:Intervertebral disc degeneration:0.31), (PRAMIPEXOLE:Intervertebral disc degeneration:0.02), (PROGESTERONE:Intervertebral disc degeneration:0.32), (RABEPRAZOLE:Intervertebral disc degeneration:0.07), (SACUBITRILVALSARTAN:Intervertebral disc degeneration:0.24), (SECUKINUMAB:Intervertebral disc degeneration:0.71), (TERIFLUNOMIDE:Intervertebral disc degeneration:0.49), (TETRACYCLINE:Intervertebral disc degeneration:0.55), (THYROID, PORCINE:Intervertebral disc degeneration:0.32), (TOCILIZUMAB:Intervertebral disc degeneration:0.6), (VILAZODONE HYDROCHLORIDE:Intervertebral disc degeneration:0.41), (VITAMIN A:Intervertebral disc degeneration:0.02), (ZOPICLONE:Intervertebral disc degeneration:0.03), (ACETAMINOPHENCODEINE PHOSPHATE:Intervertebral disc disorder:0.03), (ATENOLOL:Intervertebral disc disorder:0.24), (EVOLOCUMAB:Intervertebral disc disorder:0.05), (IRON:Intervertebral disc disorder:0.07), (VARENICLINE TARTRATE:Intervertebral disc disorder:0.19), (ACARBOSE:Intervertebral disc protrusion:0.03), (ALENDRONIC ACID:Intervertebral disc protrusion:0.12), (AMOXICILLINCLAVULANATE POTASSIUM:Intervertebral disc protrusion:0.04), (ANASTROZOLE:Intervertebral disc protrusion:1.12), (BENAZEPRIL HYDROCHLORIDE:Intervertebral disc protrusion:0.65), (BISACODYL:Intervertebral disc protrusion:0.06), (BUDESONIDEFORMOTEROL FUMARATE:Intervertebral disc protrusion:0.34), (BUMETANIDE:Intervertebral disc protrusion:0.35), (CALCIUM CARBONATEVITAMIN D:Intervertebral disc protrusion:0.38), (CARVEDILOL PHOSPHATE:Intervertebral disc protrusion:0.47), (CHONDROITIN SULFATE (BOVINE)GLUCOSAMINE HYDROCHLORIDE:Intervertebral disc protrusion:0), (CIMETIDINE:Intervertebral disc protrusion:0.03), (CLOTRIMAZOLE:Intervertebral disc protrusion:0.44), (CLOZAPINE:Intervertebral disc protrusion:0.59), (COD LIVER OIL:Intervertebral disc protrusion:0.02), (DEVICE:Intervertebral disc protrusion:0.61), (DIPHENHYDRAMINE:Intervertebral disc protrusion:0.43), (DOFETILIDE:Intervertebral disc protrusion:0.08), (DONEPEZIL:Intervertebral disc protrusion:0.01), (DOXAZOSINDOXAZOSIN MESYLATE:Intervertebral disc protrusion:0.47), (DOXEPIN:Intervertebral disc protrusion:0.02), (DOXEPIN HYDROCHLORIDE:Intervertebral disc protrusion:0.35), (EMPAGLIFLOZINLINAGLIPTIN:Intervertebral disc protrusion:0.17), (ENOXAPARIN:Intervertebral disc protrusion:0.64), (ERYTHROPOIETIN:Intervertebral disc protrusion:0.09), (EVOLOCUMAB:Intervertebral disc protrusion:1.42), (FERROUS SULFATE:Intervertebral disc protrusion:1.24), (FLECAINIDE:Intervertebral disc protrusion:0.32), (FLUTICASONE FUROATE:Intervertebral disc protrusion:0.22), (FLUTICASONE FUROATEVILANTEROL TRIFENATATE:Intervertebral disc protrusion:0.28), (GLYBURIDE:Intervertebral disc protrusion:0.56), (HYDROXYCHLOROQUINE DIPHOSPHATE:Intervertebral disc protrusion:0.53), (INDOMETHACIN:Intervertebral disc protrusion:0.75), (IPRATROPIUM:Intervertebral disc protrusion:0.4), (LABETALOLLABETALOL HYDROCHLORIDE:Intervertebral disc protrusion:0.52), (LEDIPASVIRSOFOSBUVIR:Intervertebral disc protrusion:0.59), (LERCANIDIPINE:Intervertebral disc protrusion:0.55), (LERCANIDIPINE HYDROCHLORIDE:Intervertebral disc protrusion:0.29), (LEVALBUTEROL HYDROCHLORIDE:Intervertebral disc protrusion:0.16), (METHENAMINE HIPPURATE:Intervertebral disc protrusion:0.39), (NEBIVOLOL:Intervertebral disc protrusion:0.27), (NORETHINDRONE:Intervertebral disc protrusion:0.1), (OXCARBAZEPINE:Intervertebral disc protrusion:1.55), (PAROXETINEPAROXETINE HYDROCHLORIDE:Intervertebral disc protrusion:0.55), (PEGINTERFERON BETA-1A:Intervertebral disc protrusion:0.98), (PENICILLIN:Intervertebral disc protrusion:0.59), (PERINDOPRIL:Intervertebral disc protrusion:0.3), (PLANTAGO SEED:Intervertebral disc protrusion:0.04), (POLYETHYLENE GLYCOLS:Intervertebral disc protrusion:0.82), (PROGESTERONE:Intervertebral disc protrusion:0.42), (PYRIDOXINE:Intervertebral disc protrusion:0.03), (PYRIDOXINE HYDROCHLORIDE:Intervertebral disc protrusion:0.33), (RALOXIFENE HYDROCHLORIDE:Intervertebral disc protrusion:0.58), (RANOLAZINE:Intervertebral disc protrusion:0.97), (ROFLUMILAST:Intervertebral disc protrusion:0.23), (SOTALOL:Intervertebral disc protrusion:0.77), (SULFAMETHOXAZOLE:Intervertebral disc protrusion:0.31), (TERBUTALINE SULFATE:Intervertebral disc protrusion:0.06), (TETRACYCLINE:Intervertebral disc protrusion:0.27), (TIMOLOL:Intervertebral disc protrusion:0.03), (UMECLIDINIUM BROMIDEVILANTEROL TRIFENATATE:Intervertebral disc protrusion:0.5), (PHENYLEPHRINE HYDROCHLORIDETROPICAMIDE:Intraocular lens implant:0.23), (ACETAMINOPHENCODEINE PHOSPHATE:Intraocular pressure increased:0.41), (AMIODARONE HYDROCHLORIDE:Intraocular pressure increased:0.46), (ANASTROZOLE:Intraocular pressure increased:0.26), (BUMETANIDE:Intraocular pressure increased:0.3), (CANDESARTAN CILEXETIL:Intraocular pressure increased:0.59), (CARBOMER:Intraocular pressure increased:0.02), (CARBOXYMETHYLCELLULOSE SODIUMHYPROMELLOSES:Intraocular pressure increased:0.44), (COLCHICINE:Intraocular pressure increased:0.27), (DASATINIB:Intraocular pressure increased:0.11), (DEVICE:Intraocular pressure increased:0.15), (DEXAMETHASONENEOMYCINPOLYMYXIN B SULFATE:Intraocular pressure increased:0.12), (DIPHENHYDRAMINE:Intraocular pressure increased:0.13), (DUTASTERIDE:Intraocular pressure increased:0.59), (ENOXAPARIN:Intraocular pressure increased:0.12), (ESZOPICLONE:Intraocular pressure increased:0.1), (EVEROLIMUS:Intraocular pressure increased:0.21), (EVOLOCUMAB:Intraocular pressure increased:0.59), (FERROUS SULFATEFERROUS SULFATE, DRIED:Intraocular pressure increased:0.74), (HEPARIN SODIUM:Intraocular pressure increased:0.14), (HYDROCHLOROTHIAZIDEIRBESARTAN:Intraocular pressure increased:0.13), (HYDROXYPROPYL CELLULOSE:Intraocular pressure increased:0.02), (IBRUTINIB:Intraocular pressure increased:0.64), (INDAPAMIDEPERINDOPRIL:Intraocular pressure increased:0.02), (ISOSORBIDE MONONITRATE:Intraocular pressure increased:0.23), (METAXALONE:Intraocular pressure increased:0.22), (METHYLPREDNISOLONE SODIUM SUCCINATE:Intraocular pressure increased:0.2), (METRONIDAZOLE:Intraocular pressure increased:0.17), (MOXIFLOXACINTRIAMCINOLONE:Intraocular pressure increased:0.2), (NAPHAZOLINE HYDROCHLORIDE:Intraocular pressure increased:0.14), (NATAMYCIN:Intraocular pressure increased:0.03), (NILOTINIB:Intraocular pressure increased:0.01), (OMEGA-3-ACID ETHYL ESTERS:Intraocular pressure increased:0.45), (OXYGEN:Intraocular pressure increased:0.28), (PANTOPRAZOLE MAGNESIUM:Intraocular pressure increased:0.25), (POLYETHYLENE GLYCOL 400:Intraocular pressure increased:0.57), (POVIDONE:Intraocular pressure increased:1.07), (PREDNISOLONE ACETATESULFACETAMIDE SODIUM:Intraocular pressure increased:0.27), (RABEPRAZOLE SODIUM:Intraocular pressure increased:0.41), (RANOLAZINE:Intraocular pressure increased:0.7), (SECUKINUMAB:Intraocular pressure increased:0.39), (TEMAZEPAM:Intraocular pressure increased:0.22), (TERIFLUNOMIDE:Intraocular pressure increased:0.21), (TETRACAINE HYDROCHLORIDE:Intraocular pressure increased:0.68), (TETRACAINETETRACAINE HYDROCHLORIDE:Intraocular pressure increased:0.87), (TIZANIDINE HYDROCHLORIDE:Intraocular pressure increased:0.02), (TRAMADOL HYDROCHLORIDE:Intraocular pressure increased:0.13), (VARENICLINE TARTRATE:Intraocular pressure increased:0.19), (VITAMIN B:Intraocular pressure increased:0.3), (ISOFLURANE:Intraoperative care:0.24), (VECURONIUM BROMIDE:Intraoperative care:0.03), (BICALUTAMIDE:Invasive ductal breast carcinoma:0.37), (BUMETANIDE:Invasive ductal breast carcinoma:0.46), (CANDESARTAN CILEXETILHYDROCHLOROTHIAZIDE:Invasive ductal breast carcinoma:0.14), (CEPHALEXIN:Invasive ductal breast carcinoma:0.08), (CERTOLIZUMAB PEGOL:Invasive ductal breast carcinoma:0.12), (CHOLESTYRAMINE:Invasive ductal breast carcinoma:0.07), (CLINDAMYCINCLINDAMYCIN PHOSPHATE:Invasive ductal breast carcinoma:0.33), (CLONIDINE:Invasive ductal breast carcinoma:0.14), (DEXLANSOPRAZOLE:Invasive ductal breast carcinoma:0.66), (EPINEPHRINE:Invasive ductal breast carcinoma:0.04), (ESZOPICLONE:Invasive ductal breast carcinoma:0), (EVOLOCUMAB:Invasive ductal breast carcinoma:0.15), (FERROUS SULFATEFERROUS SULFATE, DRIED:Invasive ductal breast carcinoma:0.51), (INDAPAMIDEPERINDOPRIL:Invasive ductal breast carcinoma:0.16), (IRINOTECAN HYDROCHLORIDE:Invasive ductal breast carcinoma:0.06), (NIFEDIPINE:Invasive ductal breast carcinoma:0.15), (NILOTINIB:Invasive ductal breast carcinoma:0.47), (NITROFURANTOIN:Invasive ductal breast carcinoma:0.18), (OXYGEN:Invasive ductal breast carcinoma:0.02), (PAROXETINE:Invasive ductal breast carcinoma:0.07), (PAZOPANIB HYDROCHLORIDE:Invasive ductal breast carcinoma:0.16), (POLYETHYLENE GLYCOLS:Invasive ductal breast carcinoma:0.29), (SACUBITRILVALSARTAN:Invasive ductal breast carcinoma:0.38), (TAMSULOSIN:Invasive ductal breast carcinoma:0.5), (THALIDOMIDE:Invasive ductal breast carcinoma:0.55), (TOPIRAMATE:Invasive ductal breast carcinoma:0.51), (GADOVERSETAMIDE:Investigation:0.21), (ETHAMBUTOL HYDROCHLORIDE:Iritis:0.86), (.ALPHA.1-PROTEINASE INHIBITOR HUMAN:Iron deficiency:0.05), (ACETAMINOPHENOXYCODONEOXYCODONE HYDROCHLORIDE:Iron deficiency:0.53), (ACETAZOLAMIDE:Iron deficiency:0.65), (AGALSIDASE BETA:Iron deficiency:0.49), (ALCOHOL:Iron deficiency:0.14), (ALEMTUZUMAB:Iron deficiency:0.04), (ALISKIREN HEMIFUMARATE:Iron deficiency:0.15), (AMILORIDE HYDROCHLORIDEHYDROCHLOROTHIAZIDE:Iron deficiency:0.39), (AMISULPRIDE:Iron deficiency:0.1), (AMLODIPINE BESYLATEHYDROCHLOROTHIAZIDEVALSARTAN:Iron deficiency:0.22), (AMLODIPINE BESYLATEOLMESARTAN MEDOXOMIL:Iron deficiency:0.39), (AMLODIPINEVALSARTAN:Iron deficiency:0.14), (AMPHOTERICIN B:Iron deficiency:0.14), (APREPITANT:Iron deficiency:0.43), (ATOVAQUONE:Iron deficiency:0.02), (AZACITIDINE:Iron deficiency:0.07), (AZILSARTAN KAMEDOXOMIL:Iron deficiency:0.33), (BIFIDOBACTERIUM LONGUM SUBSP. INFANTIS:Iron deficiency:0.11), (BRINZOLAMIDE:Iron deficiency:0.41), (BUDESONIDEFORMOTEROL FUMARATE:Iron deficiency:0.33), (BUPRENORPHINE HYDROCHLORIDE:Iron deficiency:0.6), (BUPRENORPHINE HYDROCHLORIDENALOXONE HYDROCHLORIDE:Iron deficiency:0.45), (BUTYLSCOPOLAMINE BROMIDE:Iron deficiency:0.48), (CALCIPOTRIENE:Iron deficiency:0.19), (CALCITONIN:Iron deficiency:0.25), (CANAGLIFLOZINMETFORMIN HYDROCHLORIDE:Iron deficiency:0.38), (CAPTOPRIL:Iron deficiency:0.62), (CARVEDILOL PHOSPHATE:Iron deficiency:0.98), (CEFOTAXIME SODIUM:Iron deficiency:0.22), (CETIRIZINE HYDROCHLORIDEPSEUDOEPHEDRINE HYDROCHLORIDE:Iron deficiency:0.08), (CICLESONIDE:Iron deficiency:0.16), (CILASTATIN SODIUMIMIPENEM:Iron deficiency:0.62), (CILOSTAZOL:Iron deficiency:0.49), (CLOMIPRAMINE HYDROCHLORIDE:Iron deficiency:0.12), (COD LIVER OIL:Iron deficiency:0.12), (CORTICOTROPIN:Iron deficiency:0.08), (CRIZOTINIB:Iron deficiency:0.19), (DACLIZUMAB:Iron deficiency:0.67), (DAPAGLIFLOZIN PROPANEDIOL:Iron deficiency:0.34), (DAPSONE:Iron deficiency:0.05), (DASABUVIROMBITASVIRPARITAPREVIRRITONAVIR:Iron deficiency:0.16), (DEFLAZACORT:Iron deficiency:0.39), (DEVICE:Iron deficiency:0.44), (DEXAMETHASONE ACETATE:Iron deficiency:0.21), (DONEPEZIL:Iron deficiency:0.1), (DOXEPIN HYDROCHLORIDE:Iron deficiency:0.33), (DRONABINOL:Iron deficiency:0.58), (DULAGLUTIDE:Iron deficiency:0), (DUTASTERIDETAMSULOSIN HYDROCHLORIDE:Iron deficiency:0.31), (ECONAZOLE:Iron deficiency:0.83), (ELETRIPTAN HYDROBROMIDE:Iron deficiency:1.14), (EMPAGLIFLOZINLINAGLIPTIN:Iron deficiency:0.37), (EPIRUBICIN:Iron deficiency:0.05), (EVOLOCUMAB:Iron deficiency:1.23), (FEXOFENADINEPSEUDOEPHEDRINE:Iron deficiency:0.11), (FORMOTEROL:Iron deficiency:0.27), (FULVESTRANT:Iron deficiency:0.57), (FUSIDIC ACID:Iron deficiency:0.81), (GLYBURIDEMETFORMIN HYDROCHLORIDE:Iron deficiency:0.01), (HALOPERIDOL:Iron deficiency:0.52), (HYDROXYCHLOROQUINE DIPHOSPHATE:Iron deficiency:0.34), (ILOPROST:Iron deficiency:0.42), (INDAPAMIDEPERINDOPRIL:Iron deficiency:1.42), (INFLIXIMAB-DYYB:Iron deficiency:0.26), (INSULIN BEEF:Iron deficiency:0.01), (INTERFERON:Iron deficiency:0.1), (INTERFERON ALFA-2A:Iron deficiency:0.25), (INTERFERON ALFA-2B:Iron deficiency:0.03), (IRINOTECAN HYDROCHLORIDE:Iron deficiency:0.23), (KETOPROFEN:Iron deficiency:0.04), (LERCANIDIPINE HYDROCHLORIDE:Iron deficiency:1.58), (LOMITAPIDE MESYLATE:Iron deficiency:0.28), (METHIMAZOLE:Iron deficiency:0.82), (METHYLPREDNISOLONE ACETATE:Iron deficiency:0.79), (MIDODRINE:Iron deficiency:0.64), (MORPHINE SULFATENALTREXONE HYDROCHLORIDE:Iron deficiency:0.25), (NABUMETONE:Iron deficiency:0.48), (NIRAPARIB:Iron deficiency:0.17), (NORTRIPTYLINE HYDROCHLORIDE:Iron deficiency:0.07), (OLAPARIB:Iron deficiency:0.04), (OLODATEROL HYDROCHLORIDETIOTROPIUM BROMIDE MONOHYDRATE:Iron deficiency:0.64), (OMEGA-3-ACID ETHYL ESTERS:Iron deficiency:1.6), (OXCARBAZEPINE:Iron deficiency:1.99), (PANTOPRAZOLE MAGNESIUM:Iron deficiency:1.41), (PAROXETINE:Iron deficiency:0.98), (PATIROMER:Iron deficiency:0.45), (PEMETREXED:Iron deficiency:0.11), (PEMETREXED DISODIUM:Iron deficiency:0.55), (PENICILLIN:Iron deficiency:1), (PHENOBARBITAL:Iron deficiency:0.74), (PHENYTOIN SODIUM:Iron deficiency:0.3), (PIMECROLIMUS:Iron deficiency:0.19), (PONATINIB:Iron deficiency:1.08), (PRASUGREL HYDROCHLORIDE:Iron deficiency:0.08), (PREDNISOLONE ACETATE:Iron deficiency:0.29), (PREDNISOLONEPREDNISOLONE ACETATEPREDNISOLONE SODIUM SUCCINATE:Iron deficiency:0.58), (PROPAFENONE:Iron deficiency:0.06), (RABEPRAZOLE:Iron deficiency:0.79), (RALOXIFENE HYDROCHLORIDE:Iron deficiency:0.75), (REGORAFENIB:Iron deficiency:0.51), (RIVASTIGMINE:Iron deficiency:0.41), (RIZATRIPTAN:Iron deficiency:0.08), (SODIUM PHOSPHATE, DIBASICSODIUM PHOSPHATE, MONOBASIC:Iron deficiency:0.11), (SULFAMETHOXAZOLE:Iron deficiency:1.23), (THYROID, PORCINE:Iron deficiency:0.23), (TROSPIUM CHLORIDE:Iron deficiency:0.33), (UMECLIDINIUM BROMIDE:Iron deficiency:0.05), (ZINC:Iron deficiency:0.38), (.ALPHA.1-PROTEINASE INHIBITOR HUMAN:Iron deficiency anaemia:0.66), (ABIRATERONE ACETATE:Iron deficiency anaemia:0.22), (ACETAMINOPHENOXYCODONEOXYCODONE HYDROCHLORIDE:Iron deficiency anaemia:0.4), (ACETAZOLAMIDE:Iron deficiency anaemia:0.69), (ACLIDINIUM BROMIDE:Iron deficiency anaemia:0.04), (ADO-TRASTUZUMAB EMTANSINE:Iron deficiency anaemia:0.94), (AGALSIDASE BETA:Iron deficiency anaemia:0.26), (AMANTADINE:Iron deficiency anaemia:0.67), (AMISULPRIDE:Iron deficiency anaemia:0.58), (AMLODIPINE BESYLATECANDESARTAN CILEXETIL:Iron deficiency anaemia:0.45), (AMLODIPINEVALSARTAN:Iron deficiency anaemia:0.09), (AMMONIUM GLYCYRRHIZATEGLYCINEMETHIONINE:Iron deficiency anaemia:0.56), (AMOXAPINE:Iron deficiency anaemia:0.87), (APRINDINE HYDROCHLORIDE:Iron deficiency anaemia:0.37),(ARMODAFINIL:Iron deficiency anaemia:0.94), (ASPIRINLANSOPRAZOLE:Iron deficiency anaemia:0.36), (ASPIRINMAGNESIUM OXIDE:Iron deficiency anaemia:0.05), (BENAZEPRILBENAZEPRIL HYDROCHLORIDE:Iron deficiency anaemia:0.54), (BENPROPERINE PHOSPHATE:Iron deficiency anaemia:0.16), (BEPOTASTINE:Iron deficiency anaemia:0.68), (BERBERINEGERANIUM:Iron deficiency anaemia:0.05), (BETAMETHASONE BUTYRATE PROPIONATE:Iron deficiency anaemia:0.45), (BETAMETHASONE DIPROPIONATECALCIPOTRIENECALCIPOTRIENE HYDRATE:Iron deficiency anaemia:0.1), (BETAMETHASONE SODIUM PHOSPHATENEOMYCIN SULFATE:Iron deficiency anaemia:0.15), (BEZAFIBRATE:Iron deficiency anaemia:0.12), (BIXALOMER:Iron deficiency anaemia:0.01), (BRINZOLAMIDE:Iron deficiency anaemia:0.21), (BROMHEXINE HYDROCHLORIDE:Iron deficiency anaemia:0.15), (BUDESONIDEFORMOTEROL FUMARATE:Iron deficiency anaemia:0.39), (BUPRENORPHINE HYDROCHLORIDENALOXONE HYDROCHLORIDE:Iron deficiency anaemia:0.57), (CALCITONIN:Iron deficiency anaemia:0.05), (CALCITONIN SALMON:Iron deficiency anaemia:0.87), (CAMOSTAT MESYLATE:Iron deficiency anaemia:0.47), (CAMPHORMENTHOLMETHYL SALICYLATE:Iron deficiency anaemia:1.1), (CANDESARTAN CILEXETILHYDROCHLOROTHIAZIDE:Iron deficiency anaemia:1.56), (CARVEDILOL PHOSPHATE:Iron deficiency anaemia:1.23), (CEFCAPENE PIVOXIL HYDROCHLORIDE:Iron deficiency anaemia:1.13), (CETIRIZINE HYDROCHLORIDEPSEUDOEPHEDRINE HYDROCHLORIDE:Iron deficiency anaemia:0.07), (CHLORHEXIDINE:Iron deficiency anaemia:0.06), (CHLOROTHIAZIDE:Iron deficiency anaemia:0.01), (CHOLESTYRAMINE:Iron deficiency anaemia:1.6), (CIMETIDINE:Iron deficiency anaemia:0.86), (CLENBUTEROL HYDROCHLORIDE:Iron deficiency anaemia:0.74), (CLINDAMYCIN:Iron deficiency anaemia:1.04), (CLOTRIMAZOLE:Iron deficiency anaemia:1.07), (COLESTILAN CHLORIDE:Iron deficiency anaemia:0.23), (CORTICOTROPIN:Iron deficiency anaemia:0.16), (CRIZOTINIB:Iron deficiency anaemia:1.21), (CROTAMITON:Iron deficiency anaemia:0.5), (CYPROHEPTADINE:Iron deficiency anaemia:0.56), (DABIGATRAN:Iron deficiency anaemia:0.7), (DABRAFENIB:Iron deficiency anaemia:0.05), (DACLATASVIR:Iron deficiency anaemia:0.04), (DALTEPARIN:Iron deficiency anaemia:0.6), (DAPAGLIFLOZIN PROPANEDIOL:Iron deficiency anaemia:0.44), (DEFLAZACORT:Iron deficiency anaemia:0.96), (DEGARELIX:Iron deficiency anaemia:0.31), (DESVENLAFAXINE SUCCINATE:Iron deficiency anaemia:0.11), (DIFLUCORTOLONE VALERATELIDOCAINE:Iron deficiency anaemia:0.17), (DIMEMORFAN PHOSPHATE:Iron deficiency anaemia:0.4), (DIPYRONE:Iron deficiency anaemia:0.24), (DOCUSATE CALCIUM:Iron deficiency anaemia:0.47), (DOFETILIDE:Iron deficiency anaemia:0.58), (DOXEPIN HYDROCHLORIDE:Iron deficiency anaemia:0), (DRONABINOL:Iron deficiency anaemia:0.65), (ECONAZOLE:Iron deficiency anaemia:0.03), (EDARAVONE:Iron deficiency anaemia:0.01), (ELCATONIN:Iron deficiency anaemia:0.14), (EMPAGLIFLOZINLINAGLIPTIN:Iron deficiency anaemia:0.64), (EPHEDRA SINICA STEMHERBALS:Iron deficiency anaemia:0.04), (ERYTHROMYCIN LACTOBIONATE:Iron deficiency anaemia:0.63), (ESTROGENS, CONJUGATEDMEDROXYPROGESTERONE ACETATE:Iron deficiency anaemia:0.63), (ETRETINATE:Iron deficiency anaemia:1.17), (EZETIMIBESIMVASTATIN:Iron deficiency anaemia:0.61), (FENOFIBRIC ACID:Iron deficiency anaemia:0.24), (FISH OIL\OMEGA-3 FATTY ACIDS:Iron deficiency anaemia:0), (FLAVIN ADENINE DINUCLEOTIDE:Iron deficiency anaemia:1.35), (FLECAINIDE:Iron deficiency anaemia:1.37), (FLOMOXEF SODIUM:Iron deficiency anaemia:0.09), (FLUDROCORTISONE ACETATE:Iron deficiency anaemia:0.37), (FLUOCINONIDE:Iron deficiency anaemia:0.11), (FLUVOXAMINE MALEATE:Iron deficiency anaemia:0), (FORMOTEROL:Iron deficiency anaemia:0.03), (FORMOTEROL FUMARATE:Iron deficiency anaemia:0.64), (FORMOTEROL FUMARATEMOMETASONE FUROATE:Iron deficiency anaemia:0.06), (FOSFOMYCIN CALCIUM:Iron deficiency anaemia:0.65), (FURSULTIAMINE:Iron deficiency anaemia:0.73), (GENTAMICIN:Iron deficiency anaemia:0.25), (GLUTAMINESODIUM GUALENATE:Iron deficiency anaemia:0.32), (GLYCOPYRROLATE:Iron deficiency anaemia:0.2), (GLYCOPYRRONIUMINDACATEROL:Iron deficiency anaemia:0.58), (GUANFACINE:Iron deficiency anaemia:0.09), (HYDROCHLOROTHIAZIDELOSARTAN:Iron deficiency anaemia:0.16), (HYDROCORTISONE ACETATE:Iron deficiency anaemia:0.56), (HYDROXYCHLOROQUINE DIPHOSPHATE:Iron deficiency anaemia:0.04), (ILOPROST:Iron deficiency anaemia:1.2), (IMIDAPRIL HYDROCHLORIDE:Iron deficiency anaemia:0.23), (IMIPRAMINE HYDROCHLORIDE:Iron deficiency anaemia:0.65), (IMMUNE GLOBULIN NOS:Iron deficiency anaemia:0.8), (INDACATEROL:Iron deficiency anaemia:0.06), (INDAPAMIDEPERINDOPRIL:Iron deficiency anaemia:0.82), (INSULIN BEEF:Iron deficiency anaemia:0.15), (IPRAGLIFLOZIN L-PROLINE:Iron deficiency anaemia:0.15), (IXAZOMIB:Iron deficiency anaemia:0.41), (KALLIDINOGENASE:Iron deficiency anaemia:0.29), (LABETALOL HYDROCHLORIDE:Iron deficiency anaemia:0.46), (LACTOBACILLUS RHAMNOSUS:Iron deficiency anaemia:0.28), (LANDIOLOL HYDROCHLORIDE:Iron deficiency anaemia:0.35), (LAPATINIB DITOSYLATE:Iron deficiency anaemia:0.2), (LENOGRASTIM:Iron deficiency anaemia:0.24), (LERCANIDIPINE:Iron deficiency anaemia:1.52), (LERCANIDIPINE HYDROCHLORIDE:Iron deficiency anaemia:1.15), (LICORICE ROOT EXTRACTRHUBARB:Iron deficiency anaemia:0), (LINACLOTIDE:Iron deficiency anaemia:0.04), (LITHIUM:Iron deficiency anaemia:0.05), (LOMITAPIDE MESYLATE:Iron deficiency anaemia:0.36), (LULICONAZOLE:Iron deficiency anaemia:0.72), (MECLIZINEMECLIZINE HYDROCHLORIDE:Iron deficiency anaemia:0.23), (MEPOLIZUMAB:Iron deficiency anaemia:0.2), (METFORMIN HYDROCHLORIDEVILDAGLIPTIN:Iron deficiency anaemia:0.41), (METHENAMINE HIPPURATE:Iron deficiency anaemia:0.5), (METHENOLONE ENANTHATE:Iron deficiency anaemia:0.89), (METHYLPREDNISOLONE ACETATE:Iron deficiency anaemia:0.6), (MICONAZOLE NITRATE:Iron deficiency anaemia:0.01), (MIGLUSTAT:Iron deficiency anaemia:0.2), (MITIGLINIDE CALCIUM HYDRATEVOGLIBOSE:Iron deficiency anaemia:0.47), (MOSAPRIDE CITRATE DIHYDRATE:Iron deficiency anaemia:0.26), (MOXONIDINE:Iron deficiency anaemia:0.25), (NADROPARIN CALCIUM:Iron deficiency anaemia:0.39), (NICARDIPINE:Iron deficiency anaemia:0.51), (NIRAPARIB:Iron deficiency anaemia:0.22), (NORTRIPTYLINE:Iron deficiency anaemia:0.66), (OLODATEROL HYDROCHLORIDETIOTROPIUM BROMIDE MONOHYDRATE:Iron deficiency anaemia:0.51), (OXYBUTYNIN CHLORIDE:Iron deficiency anaemia:0.54), (PANTOPRAZOLE MAGNESIUM:Iron deficiency anaemia:1.38), (PATIROMER:Iron deficiency anaemia:0.31), (PEGINTERFERON BETA-1A:Iron deficiency anaemia:0.57), (PEMETREXED:Iron deficiency anaemia:0.66), (PENICILLIN G:Iron deficiency anaemia:0.11), (PENTAZOCINE:Iron deficiency anaemia:0.1), (PENTOXIFYLLINE:Iron deficiency anaemia:0.03), (PILSICAINIDE HYDROCHLORIDE:Iron deficiency anaemia:1.84), (PIROXICAM:Iron deficiency anaemia:0.37), (POLYSTYRENE:Iron deficiency anaemia:0.6), (POLYSTYRENE SULFONIC ACID:Iron deficiency anaemia:0.24), (POTASSIUM CITRATETRISODIUM CITRATE DIHYDRATE:Iron deficiency anaemia:0.93), (PREDNISOLONE ACETATE:Iron deficiency anaemia:0.04), (PRIMIDONE:Iron deficiency anaemia:1.18), (PROPIVERINE:Iron deficiency anaemia:0.13), (PSYLLIUM HUSK:Iron deficiency anaemia:0.02), (PYRAZINAMIDE:Iron deficiency anaemia:0.76), (PYRIDOXINE:Iron deficiency anaemia:0.34), (QUERCUS SALICINA STEM:Iron deficiency anaemia:0.07), (REGORAFENIB:Iron deficiency anaemia:0.97), (RETINOL:Iron deficiency anaemia:0.05), (ROFLUMILAST:Iron deficiency anaemia:1.07), (SELEXIPAG:Iron deficiency anaemia:0.53), (SIVELESTAT SODIUM:Iron deficiency anaemia:0.09), (SODIUM FLUORIDE:Iron deficiency anaemia:0.15), (SODIUM GUALENATE MONOHYDRATE:Iron deficiency anaemia:0.08), (SODIUM PHOSPHATE, DIBASICSODIUM PHOSPHATE, MONOBASIC:Iron deficiency anaemia:0.06), (SULFAMETHOXAZOLE:Iron deficiency anaemia:1.06), (SUMATRIPTANSUMATRIPTAN SUCCINATE:Iron deficiency anaemia:0.79), (SUPLATAST TOSILATE:Iron deficiency anaemia:0.41), (TEDUGLUTIDEWATER:Iron deficiency anaemia:0.2), (TERBUTALINE SULFATE:Iron deficiency anaemia:1), (TERIFLUNOMIDE:Iron deficiency anaemia:0.57), (THYROID, PORCINE:Iron deficiency anaemia:0.07), (TINZAPARIN:Iron deficiency anaemia:0.37), (TIPEPIDINE HIBENZATE:Iron deficiency anaemia:1.04), (TOCOPHEROL:Iron deficiency anaemia:0.14), (TRABECTEDIN:Iron deficiency anaemia:0.08), (TRAMETINIB:Iron deficiency anaemia:0.2), (TRIAMTERENE:Iron deficiency anaemia:0.62), (VANCOMYCIN HYDROCHLORIDE:Iron deficiency anaemia:0.39), (VARENICLINE TARTRATE:Iron deficiency anaemia:1.17), (ZINC:Iron deficiency anaemia:0.25), (ZONISAMIDE:Iron deficiency anaemia:0.8), (ALENDRONIC ACID:Iron overload:0.36), (ANASTROZOLE:Iron overload:0.85), (BICALUTAMIDE:Iron overload:0.05), (BISACODYL OR DOCUSATE SODIUM:Iron overload:0.17), (BUDESONIDEFORMOTEROL FUMARATE:Iron overload:0.04), (CANAKINUMAB:Iron overload:0.39), (CANDESARTAN CILEXETILHYDROCHLOROTHIAZIDE:Iron overload:0.44), (CARFILZOMIB:Iron overload:0.12), (CERTOLIZUMAB PEGOL:Iron overload:0.4), (CHOLESTYRAMINE:Iron overload:0.45), (CILASTATIN SODIUMIMIPENEM:Iron overload:0.06), (CLOTRIMAZOLE:Iron overload:0.04), (CORTISONEHYDROCORTISONE:Iron overload:0.4), (DEVICE:Iron overload:0.16), (DEXLANSOPRAZOLE:Iron overload:0.91), (DICLOFENAC SODIUM:Iron overload:1.01), (DILTIAZEM HYDROCHLORIDE:Iron overload:1.3), (DOXAZOSINDOXAZOSIN MESYLATE:Iron overload:0.38), (DUTASTERIDE:Iron overload:0.77), (EDOXABAN TOSYLATE:Iron overload:0.19), (ENALAPRIL MALEATE:Iron overload:0.35), (ENOXAPARIN:Iron overload:1.09), (ESZOPICLONE:Iron overload:0.65), (EVEROLIMUS:Iron overload:0.87), (EVOLOCUMAB:Iron overload:0.35), (EZETIMIBE:Iron overload:0.49), (EZETIMIBESIMVASTATIN:Iron overload:0.07), (FESOTERODINE FUMARATE:Iron overload:0.18), (FLECAINIDE:Iron overload:0.23), (FLUTICASONE:Iron overload:0.05), (FLUTICASONE FUROATEVILANTEROL TRIFENATATE:Iron overload:0.06), (GLYCOPYRRONIUMINDACATEROL:Iron overload:0.11), (HEPARIN SODIUM:Iron overload:0.32), (HYDROCHLOROTHIAZIDEIRBESARTAN:Iron overload:0.33), (IDELALISIB:Iron overload:0.39), (INDAPAMIDEPERINDOPRIL:Iron overload:0.22), (IPRATROPIUM:Iron overload:0.23), (IRON:Iron overload:0.65), (ISOSORBIDE:Iron overload:0.42), (LABETALOLLABETALOL HYDROCHLORIDE:Iron overload:0.25), (LAMOTRIGINE:Iron overload:0.35), (LEDIPASVIRSOFOSBUVIR:Iron overload:0.19), (LERCANIDIPINE HYDROCHLORIDE:Iron overload:0.21), (LEVALBUTEROL HYDROCHLORIDE:Iron overload:0.03), (LIDOCAINE HYDROCHLORIDE:Iron overload:0.02), (LORATADINE:Iron overload:0.09), (NALOXONE HYDROCHLORIDEOXYCODONE HYDROCHLORIDE:Iron overload:0.03), (OXCARBAZEPINE:Iron overload:0.74), (PITAVASTATIN CALCIUM:Iron overload:0.03), (RABEPRAZOLE:Iron overload:0.07), (SACUBITRILVALSARTAN:Iron overload:0.46), (SULFAMETHOXAZOLE:Iron overload:0.79), (TEMAZEPAM:Iron overload:0.38), (TIZANIDINE HYDROCHLORIDE:Iron overload:0.03), (TOPIRAMATE:Iron overload:1.1), (VALGANCICLOVIR HYDROCHLORIDE:Iron overload:0.35), (VITAMIN B:Iron overload:0.06), (REMIFENTANIL:Irrigation therapy:0.18), (ALCOHOL:Irritability:0.58), (ARIPIPRAZOLE LAUROXIL:Irritability:0.1), (BUPRENORPHINE HYDROCHLORIDENALOXONE HYDROCHLORIDE:Irritability:0.56), (CALCIUM CARBONATECHOLECALCIFEROL:Irritability:0.27), (CELECOXIB:Irritability:0.1), (CERTO LIZUMAB PEGOL:Irritability:0.18), (DEXLANSOPRAZOLE:Irritability:0.37), (DILTIAZEM HYDROCHLORIDE:Irritability:0.46), (EVOLOCUMAB:Irritability:0.05), (FERROUS SULFATEFERROUS SULFATE, DRIED:Irritability:0.2), (IBRUTINIB:Irritability:0.16), (LOVASTATIN:Irritability:0.08), (METR ONIDAZOLE:Irritability:0.05), (PANTOPRAZOLE:Irritability:0.23), (PAROXETINEPAROXE TINE HYDROCHLORIDE:Irritability:0.08), (PHENOBARBITAL:Irritability:0.06), (PRAMIPEXOLE: Irritability:0.23), (PROMETHAZINEPROMETHAZINE HYDROCHLORIDE:Irritability:0.05), (TERIFLUNOMIDE:Irritability:0.1), (WARFARIN SODIUM:Irritability:0.26), (ZONISAMIDE:Irritability:0.62), (ABIRATERONE ACETATE:Irritable bowel syndrome:0.33), (ACARBOSE:Irritable bowel syndrome:0.3), (ACETAMINOPHENPROPOXYPHENE NAPSYLATE:Irritable bowel syndrome:0.48), (ACETAZOLAMIDE:Irritable bowel syndrome:1.4), (ADO-TRASTUZUMAB EMTANSINE:Irritable bowel syndrome:0.53), (ALBIGLUTIDE:Irritable bowel syndrome:0.07), (ALBUTEROLIPRATROPIUM:Irritable bowel syndrome:0.06), (AMILORIDE HYDROCHLORIDEHYDROCHLOROTHIAZIDE:Irritable bowel syndrome:0.26), (AMINO ACIDSDEXTROSEELECTROLYTES NOS:Irritable bowel syndrome:1.01), (AMMONIUM CHLORIDEDEXTROMETHORPHANSODIUM CITRATE:Irritable bowel syndrome:0.19), (AMPICILLIN SODIUMSULBACTAM SODIUM:Irritable bowel syndrome:0.03), (ANTIHISTAMINES NOS:Irritable bowel syndrome:0.3), (ARIPIPRAZOLE LAUROXIL:Irritable bowel syndrome:0.12), (AXITINIB:Irritable bowel syndrome:0.35), (BACITRACIN:Irritable bowel syndrome:0.25), (BENAZEPRILBENAZEPRIL HYDROCHLORIDE:Irritable bowel syndrome:0.98), (BENZTROPINE MESYLATE:Irritable bowel syndrome:0.34), (BETAMETHASONE DIPROPIONATECALCIPOTRIENECALCIPOTRIENE HYDRATE:Irritable bowel syndrome:0.73), (BETAMETHASONECALCIPOTRIENE:Irritable bowel syndrome:0.66), (BETHANECHOL CHLORIDE:Irritable bowel syndrome:0.14), (BUDESONIDEFORMOTEROL FUMARATE:Irritable bowel syndrome:0.7), (BUPRENORPHINENALOXONE:Irritable bowel syndrome:0.16), (CALCIPOTRIENE:Irritable bowel syndrome:0.58), (CALCITONIN:Irritable bowel syndrome:0.47), (CALCIUM ACETATE:Irritable bowel syndrome:1.17), (CALCIUMMAGNESIUM:Irritable bowel syndrome:0.71), (CALCIUMMAGNESIUMZINC:Irritable bowel syndrome:0.68), (CANAGLIFLOZINMETFORMIN HYDROCHLORIDE:Irritable bowel syndrome:0.92), (CANDESARTAN CILEXETILHYDROCHLOROTHIAZIDE:Irritable bowel syndrome:1.58), (CEFAZOLIN:Irritable bowel syndrome:0.48), (CEFAZOLIN SODIUM:Irritable bowel syndrome:0.13), (CHLORPHENIRAMINEHYDROCODONE:Irritable bowel syndrome:0.49), (CLINDAMYCIN PHOSPHATE:Irritable bowel syndrome:0.72), (CLOMIPRAMINE HYDROCHLORIDE:Irritable bowel syndrome:0.21), (CLOZAPINE:Irritable bowel syndrome:1.07), (CORTICOTROPIN:Irritable bowel syndrome:0.75), (CYPROHEPTADINE:Irritable bowel syndrome:1.13), (CYPROHEPTADINE HYDROCHLORIDE:Irritable bowel syndrome:0.22), (DABIGATRAN:Irritable bowel syndrome:0.16), (DACLIZUMAB:Irritable bowel syndrome:1.2), (DANAZOL:Irritable bowel syndrome:0.62), (DANTROLENE SODIUM:Irritable bowel syndrome:0.1), (DIPYRONE:Irritable bowel syndrome:0.45), (DOLASETRON MESYLATE:Irritable bowel syndrome:0.15), (DORZOLAMIDE:Irritable bowel syndrome:0.15), (DOXYLAMINE SUCCINATE:Irritable bowel syndrome:0.3), (DROXIDOPA:Irritable bowel syndrome:0.03), (ECONAZOLE:Irritable bowel syndrome:0.07), (EDOXABAN TOSYLATE:Irritable bowel syndrome:0.85), (ETORICOXIB:Irritable bowel syndrome:0.41), (ETRETINATE:Irritable bowel syndrome:0.06), (EVOLOCUMAB:Irritable bowel syndrome:1.72), (FERROUS FUMARATE:Irritable bowel syndrome:0.04), (FERROUS GLUCONATE:Irritable bowel syndrome:0.4), (FORMOTEROL:Irritable bowel syndrome:0.06), (FORMOTEROL FUMARATEMOMETASONE FUROATE:Irritable bowel syndrome:0.54), (GLYCOPYRROLATEINDACATEROL MALEATE:Irritable bowel syndrome:0.01), (GLYCOPYRRONIUM:Irritable bowel syndrome:0.1), (GOSERELIN ACETATE:Irritable bowel syndrome:0.02), (IMATINIB MESYLATE:Irritable bowel syndrome:0.88), (INDACATEROL:Irritable bowel syndrome:0.16), (INDAPAMIDEPERINDOPRIL:Irritable bowel syndrome:0.5), (INSULIN BEEF:Irritable bowel syndrome:0.12), (INSULIN DEGLUDEC:Irritable bowel syndrome:0.18), (INTERFERON ALFA-2B:Irritable bowel syndrome:0.08), (LAPATINIB DITOSYLATE:Irritable bowel syndrome:0.27), (LERCANIDIPINE:Irritable bowel syndrome:0.85), (LEVALBUTEROL:Irritable bowel syndrome:0.06), (LOMITAPIDE MESYLATE:Irritable bowel syndrome:0.97), (MEGESTROL:Irritable bowel syndrome:0.02), (MEPOLIZUMAB:Irritable bowel syndrome:0.37), (METAMIZOLE:Irritable bowel syndrome:0.14), (METFORMIN HYDROCHLORIDEVILDAGLIPTIN:Irritable bowel syndrome:0.17), (METHIMAZOLE:Irritable bowel syndrome:0.74), (METHYLPREDNISOLONE ACETATE:Irritable bowel syndrome:1.39), (MIDODRINE HYDROCHLORIDE:Irritable bowel syndrome:0.76), (MOEXIPRIL HYDROCHLORIDE:Irritable bowel syndrome:0.07), (MORPHINE SULFATENALTREXONE HYDROCHLORIDE:Irritable bowel syndrome:0.26), (MOXIFLOXACIN:Irritable bowel syndrome:0.03), (NALOXONE:Irritable bowel syndrome:0.03), (NALOXONE HYDROCHLORIDEOXYCODONE HYDROCHLORIDE:Irritable bowel syndrome:1.15), (NALOXONE HYDROCHLORIDETILIDINE HYDROCHLORIDE:Irritable bowel syndrome:0.1), (NILOTINIB:Irritable bowel syndrome:0.92), (NIRAPARIB:Irritable bowel syndrome:0.64), (OLMESARTAN:Irritable bowel syndrome:0.29), (OXYBUTYNINOXYBUTYNIN CHLORIDE:Irritable bowel syndrome:0.31), (OXYMORPHONE:Irritable bowel syndrome:0.18), (PANTOPRAZOLE MAGNESIUM:Irritable bowel syndrome:2.18), (PATIROMER:Irritable bowel syndrome:0.43), (PAZOPANIB HYDROCHLORIDE:Irritable bowel syndrome:0.8), (PEGINTERFERON BETA-1A:Irritable bowel syndrome:1.15), (PEMETREXED DISODIUM:Irritable bowel syndrome:0.35), (PENICILLIN:Irritable bowel syndrome:1.99), (PHENYTOIN SODIUM:Irritable bowel syndrome:0.49), (POLYETHYLENE GLYCOL 3350\POTASSIUM CHLORIDESODIUM BICARBONATESODIUM CHLORIDESODIUM SULFATE ANHYDROUS:Irritable bowel syndrome:0.06), (PROTRIPTYLINE HYDROCHLORIDE:Irritable bowel syndrome:0.94), (QUINACRINE:Irritable bowel syndrome:0.16), (REGORAFENIB:Irritable bowel syndrome:0.32), (RETINOL:Irritable bowel syndrome:0.55), (RIOCIGUAT:Irritable bowel syndrome:0.77), (ROFECOXIB:Irritable bowel syndrome:0.17), (SELEGILINE:Irritable bowel syndrome:0.45), (SOFOSBUVIRVELPATASVIR:Irritable bowel syndrome:0.02), (SULFAMETHOXAZOLE:Irritable bowel syndrome:0.98), (TAMOXIFEN CITRATE:Irritable bowel syndrome:0.26), (TELOTRISTAT ETHYL:Irritable bowel syndrome:0.09), (THALIDOMIDE:Irritable bowel syndrome:0.99), (THEOPHYLLINE:Irritable bowel syndrome:0.14), (TIBOLONE:Irritable bowel syndrome:0.02), (TINZAPARIN SODIUM:Irritable bowel syndrome:0.1), (TIOTROPIUM:Irritable bowel syndrome:0.64), (TOBRAMYCIN:Irritable bowel syndrome:0.05), (UMECLIDINIUM BROMIDE:Irritable bowel syndrome:0.45), (UMECLIDINIUM BROMIDEVILANTEROL TRIFENATATE:Irritable bowel syndrome:1.31), (VALGANCICLOVIR HYDROCHLORIDE:Irritable bowel syndrome:0.68), (VINORELBINE TARTRATE:Irritable bowel syndrome:0.06), (YEAST:Irritable bowel syndrome:0.34), (ZICONOTIDE ACETATE:Irritable bowel syndrome:0.07), (ZILEUTON:Irritable bowel syndrome:0.38), (DIGOXIN:Ischaemia:0.16), (CALCIUM:Ischaemic cardiomyopathy:0.68), (CANDESARTAN CILEXETILHYDROCHLOROTHIAZIDE:Ischaemic cardiomyopathy:0.88), (CELECOXIB:Ischaemic cardiomyopathy:0.22), (DASATINIB:Ischaemic cardiomyopathy:0.46), (DEVICE:Ischaemic cardiomyopathy:0.02), (DIGITOXIN:Ischaemic cardiomyopathy:0.28), (DOCUSATE SODIUM:Ischaemic cardiomyopathy:0.25), (EVOLOCUMAB:Ischaemic cardiomyopathy:0.71), (FLUTICASONE FUROATEVILANTEROL TRIFENATATE:Ischaemic cardiomyopathy:0), (HYDROCHLOROTHIAZIDELISINOPRIL:Ischaemic cardiomyopathy:0.02), (IBRUTINIB:Ischaemic cardiomyopathy:0.43), (ILOPROST:Ischaemic cardiomyopathy:0.62), (IMATINIB MESYLATE:Ischaemic cardiomyopathy:0.7), (INDAPAMIDEPERINDOPRIL:Ischaemic cardiomyopathy:0.98), (MELOXICAM:Ischaemic cardiomyopathy:0.25), (MONTELUKAST SODIUM:Ischaemic cardiomyopathy:0.46), (NIFEDIPINE:Ischaemic cardiomyopathy:0.36), (POLYETHYLENE GLYCOLS:Ischaemic cardiomyopathy:0.32), (SECUKINUMAB:Ischaemic cardiomyopathy:0.18), (VENLAFAXINE HYDROCHLORIDE:Ischaemic cardiomyopathy:0.11), (ABIRATERONE ACETATE:Ischaemic heart disease prophylaxis:0.08), (ACETAMINOPHENOXYCODONEOXYCODONE HYDROCHLORIDE:Ischaemic heart disease prophylaxis:0.05), (ACETAMINOPHENPROPOXYPHENE HYDROCHLORIDE:Ischaemic heart disease prophylaxis:0.12), (ALENDRONIC ACID:Ischaemic heart disease prophylaxis:0.14), (ALFUZOSIN HYDROCHLORIDE:Ischaemic heart disease prophylaxis:0.05), (ALISKIREN HEMIFUMARATE:Ischaemic heart disease prophylaxis:0.06), (AMLODIPINE BESYLATEOLMESARTAN MEDOXOMIL:Ischaemic heart disease prophylaxis:0.16), (AMLODIPINEVALSARTAN:Ischaemic heart disease prophylaxis:0.19), (ARIPIPRAZOLE:Ischaemic heart disease prophylaxis:0.31),(ARMODAFINIL:Ischaemic heart disease prophylaxis:0.5), (ATROPINE SULFATEDIPHENOXYLATE HYDROCHLORIDE:Ischaemic heart disease prophylaxis:0.18), (AXITINIB:Ischaemic heart disease prophylaxis:0), (BISACODYL OR DOCUSATE SODIUM:Ischaemic heart disease prophylaxis:0.93), (BUDESONIDEFORMOTEROL FUMARATE:Ischaemic heart disease prophylaxis:0.45), (BUPRENORPHINE HYDROCHLORIDENALOXONE HYDROCHLORIDE:Ischaemic heart disease prophylaxis:0.2), (CICLESONIDE:Ischaemic heart disease prophylaxis:0.04), (CILASTATIN SODIUMIMIPENEM:Ischaemic heart disease prophylaxis:0.22), (CIMETIDINE:Ischaemic heart disease prophylaxis:0.53), (CINACALCET HYDROCHLORIDE:Ischaemic heart disease prophylaxis:0.01), (CIPROFLOXACIN HYDROCHLORIDE:Ischaemic heart disease prophylaxis:0.21), (CLINDAMYCINCLINDAMYCIN PHOSPHATE:Ischaemic heart disease prophylaxis:1.01), (CLONIDINE HYDROCHLORIDE:Ischaemic heart disease prophylaxis:0.02), (CLOTRIMAZOLE:Ischaemic heart disease prophylaxis:0.78), (CORTICOTROPIN:Ischaemic heart disease prophylaxis:0.02), (CRIZOTINIB:Ischaemic heart disease prophylaxis:0.02), (DABIGATRAN:Ischaemic heart disease prophylaxis:1.19), (DALTEPARIN SODIUM:Ischaemic heart disease prophylaxis:0.22), (DAPAGLIFLOZIN PROPANEDIOL:Ischaemic heart disease prophylaxis:0.34), (DASATINIB:Ischaemic heart disease prophylaxis:1.26), (DEVICE:Ischaemic heart disease prophylaxis:0.73), (DEXAMETHASONE ACETATE:Ischaemic heart disease prophylaxis:0.07), (DICYCLOMINE HYDROCHLORIDE:Ischaemic heart disease prophylaxis:0.8), (DOMPERIDONE:Ischaemic heart disease prophylaxis:0.33), (DOXEPIN HYDROCHLORIDE:Ischaemic heart disease prophylaxis:0.13), (DRONABINOL:Ischaemic heart disease prophylaxis:0.25), (ECONAZOLE:Ischaemic heart disease prophylaxis:0.2), (EDOXABAN TOSYLATE:Ischaemic heart disease prophylaxis:1.2), (ELETRIPTAN HYDROBROMIDE:Ischaemic heart disease prophylaxis:0.6), (EMPAGLIFLOZINLINAGLIPTIN:Ischaemic heart disease prophylaxis:0.3), (ESZOPICLONE:Ischaemic heart disease prophylaxis:0.5), (ETORICOXIB:Ischaemic heart disease prophylaxis:0.34), (FESOTERODINE FUMARATE:Ischaemic heart disease prophylaxis:0.8), (FINGOLIMOD HYDROCHLORIDE:Ischaemic heart disease prophylaxis:0.36), (FLUDROCORTISONE ACETATE:Ischaemic heart disease prophylaxis:0.23), (FLUTICASONE FUROATE:Ischaemic heart disease prophylaxis:0.1), (FORMOTEROL FUMARATE DIHYDRATEMOMETASONE FUROATE:Ischaemic heart disease prophylaxis:0.3), (GLIMEPIRIDEROSIGLITAZONE MALEATE:Ischaemic heart disease prophylaxis:0.19), (GLYCOPYRRONIUMINDACATEROL:Ischaemic heart disease prophylaxis:0.72), (HERBALSTURMERIC:Ischaemic heart disease prophylaxis:0.29), (ILOPROST:Ischaemic heart disease prophylaxis:0.45), (IMATINIB MESYLATE:Ischaemic heart disease prophylaxis:1.13), (INSULIN DEGLUDEC:Ischaemic heart disease prophylaxis:0.15), (INSULIN GLULISINE:Ischaemic heart disease prophylaxis:0.23), (IPRATROPIUM:Ischaemic heart disease prophylaxis:1.1), (LABETALOLLABETALOL HYDROCHLORIDE:Ischaemic heart disease prophylaxis:0.94), (LEDIPASVIRSOFOSBUVIR:Ischaemic heart disease prophylaxis:0.73), (LITHIUM CARBONATE:Ischaemic heart disease prophylaxis:1.26), (MEGESTROL ACETATE:Ischaemic heart disease prophylaxis:0.1), (MEROPENEM:Ischaemic heart disease prophylaxis:0), (METHENAMINE HIPPURATE:Ischaemic heart disease prophylaxis:0.17), (METHYLPREDNISOLONE ACETATE:Ischaemic heart disease prophylaxis:0.26), (METHYLPREDNISOLONE SODIUM SUCCINATE:Ischaemic heart disease prophylaxis:1.18), (METRONIDAZOLEMETRONIDAZOLE HYDROCHLORIDE:Ischaemic heart disease prophylaxis:0.29), (MIDODRINE:Ischaemic heart disease prophylaxis:0.39), (MINOCYCLINEMINOCYCLINE HYDROCHLORIDE:Ischaemic heart disease prophylaxis:0.34), (MOXIFLOXACIN HYDROCHLORIDE:Ischaemic heart disease prophylaxis:1.11), (NATEGLINIDE:Ischaemic heart disease prophylaxis:0.39), (NORTRIPTYLINE:Ischaemic heart disease prophylaxis:0.45), (OLODATEROL HYDROCHLORIDETIOTROPIUM BROMIDE MONOHYDRATE:Ischaemic heart disease prophylaxis:0.26), (OXYBUTYNIN CHLORIDE:Ischaemic heart disease prophylaxis:0.27), (PATIROMER:Ischaemic heart disease prophylaxis:0.09), (PAZOPANIB HYDROCHLORIDE:Ischaemic heart disease prophylaxis:0.19), (PEGINTERFERON BETA-1A:Ischaemic heart disease prophylaxis:0.16), (PEMETREXED DISODIUM:Ischaemic heart disease prophylaxis:0.15), (PENICILLIN:Ischaemic heart disease prophylaxis:0.2), (PHENPROCOUMON:Ischaemic heart disease prophylaxis:0.13), (PRAMIPEXOLE DIHYDROCHLORIDE:Ischaemic heart disease prophylaxis:0.73), (RABEPRAZOLE:Ischaemic heart disease prophylaxis:0.6), (ROFLUMILAST:Ischaemic heart disease prophylaxis:0.43), (SACCHAROMYCES CEREVISIAE:Ischaemic heart disease prophylaxis:0.1), (SEVELAMER CARBONATE:Ischaemic heart disease prophylaxis:0.12), (SODIUM OXYBATE:Ischaemic heart disease prophylaxis:0.77), (SUMATRIPTANSUMATRIPTAN SUCCINATE:Ischaemic heart disease prophylaxis:0.59), (TERIFLUNOMIDE:Ischaemic heart disease prophylaxis:0.38), (TETRACYCLINE:Ischaemic heart disease prophylaxis:0.48), (TOCILIZUMAB:Ischaemic heart disease prophylaxis:0.2), (TRIAMCINOLONE:Ischaemic heart disease prophylaxis:0.07), (TRIAMCINOLONE ACETONIDE:Ischaemic heart disease prophylaxis:0.73), (UMECLIDINIUM BROMIDEVILANTEROL TRIFENATATE:Ischaemic heart disease prophylaxis:0.91), (URSODIOL:Ischaemic heart disease prophylaxis:0.24), (VILAZODONE HYDROCHLORIDE:Ischaemic heart disease prophylaxis:0.05), (VITAMIN B:Ischaemic heart disease prophylaxis:1), (ALBUTEROL SULFATE:Ischaemic stroke:0.73), (CALCIUM ACETATE:Ischaemic stroke:0.08), (CALCIUMVITAMIN D:Ischaemic stroke:0.02), (CARVEDILOL PHOSPHATE:Ischaemic stroke:0.02), (CELECOXIB:Ischaemic stroke:0.75), (CEPHALEXIN:Ischaemic stroke:0.4), (CERTOLIZUMAB PEGOL:Ischaemic stroke:0.11), (CETIRIZINE HYDROCHLORIDE:Ischaemic stroke:0.35), (CLINDAMYCINCLINDAMYCIN PHOSPHATE:Ischaemic stroke:0.54), (CLOTRIMAZOLE:Ischaemic stroke:0.08), (CORTISONEHYDROCORTISONE:Ischaemic stroke:0.22), (DASATINIB:Ischaemic stroke:0.4), (DEVICE:Ischaemic stroke:0.14), (DEXLANSOPRAZOLE:Ischaemic stroke:0.3), (DICLOFENAC:Ischaemic stroke:0.63), (DIVALPROEX SODIUM:Ischaemic stroke:0.39), (DOXAZOSINDOXAZOSIN MESYLATE:Ischaemic stroke:0.3), (EVEROLIMUS:Ischaemic stroke:0.62), (FESOTERODINE FUMARATE:Ischaemic stroke:0.02), (GLYCOPYRRONIUMINDACATEROL:Ischaemic stroke:0.2), (HYDROMORPHONE HYDROCHLORIDE:Ischaemic stroke:0.25), (IBRUTINIB:Ischaemic stroke:0.92), (IMATINIB MESYLATE:Ischaemic stroke:0.28), (ISOSORBIDE:Ischaemic stroke:0.39), (LEDIPASVIRSOFOSBUVIR:Ischaemic stroke:0.18), (MAGNESIUM:Ischaemic stroke:0.53), (METFORMIN HYDROCHLORIDEROSIGLITAZONE MALEATE:Ischaemic stroke:0.1), (METHYLPREDNISOLONE ACETATE:Ischaemic stroke:0.19), (NALOXONE HYDROCHLORIDEOXYCODONE HYDROCHLORIDE:Ischaemic stroke:0.29), (NILOTINIB:Ischaemic stroke:0.35), (NITROFURANTOIN:Ischaemic stroke:0.21), (OMEGA-3-ACID ETHYL ESTERS:Ischaemic stroke:0.2), (OXYGEN:Ischaemic stroke:0.61), (PAROXETINEPAROXETINE HYDROCHLORIDE:Ischaemic stroke:0.33), (POTASSIUM:Ischaemic stroke:0.86), (PRAMIPEXOLE DIHYDROCHLORIDE:Ischaemic stroke:0.13), (PRIMIDONE:Ischaemic stroke:0.54), (PROPAFENONE:Ischaemic stroke:0.05), (RANOLAZINE:Ischaemic stroke:1.17), (RISEDRONATE SODIUM:Ischaemic stroke:0.05), (ROSIGLITAZONE MALEATE:Ischaemic stroke:0.25), (SACUBITRILVALSARTAN:Ischaemic stroke:0.92), (SECUKINUMAB:Ischaemic stroke:0.04), (TEMAZEPAM:Ischaemic stroke:0.19), (TERIFLUNOMIDE:Ischaemic stroke:0.11), (TIROFIBAN HYDROCHLORIDE:Ischaemic stroke:0.11), (DEXLANSOPRAZOLE:Joint injury:0.63), (DIVALPROEX SODIUM:Joint injury:0.12), (EVOLOCUMAB:Joint injury:0.34), (PANTOPRAZOLE:Joint injury:0.01), (PAROXETINE:Joint injury:0.1), (TOCILIZUMAB:Joint injury:0.12), (VARENICLINE TARTRATE:Joint injury:0.55), (VERAPAMIL HYDROCHLORIDE:Joint injury:0.05), (WARFARIN:Joint injury:0.06), (ACETAMINOPHENBUTALBITALCAFFEINE:Joint swelling:0.76), (ACETAMINOPHENOXYCODONEOXYCODONE HYDROCHLORIDE:Joint swelling:0.05), (AZITHROMYCIN ANHYDROUS:Joint swelling:0.17), (CALCIUM CARBONATEVITAMIN D:Joint swelling:0.04), (CANAGLIFLOZINMETFORMIN HYDROCHLORIDE:Joint swelling:0.03), (CANDESARTAN:Joint swelling:0.92), (CARVEDILOL PHOSPHATE:Joint swelling:0.69), (CICLESONIDE:Joint swelling:0.25), (CORTICOTROPIN:Joint swelling:0.01), (DALFAMPRIDINE:Joint swelling:0.45), (DAPAGLIFLOZIN PROPANEDIOL:Joint swelling:0.47), (DASATINIB:Joint swelling:0.89), (DESLORATADINE:Joint swelling:0.18), (DESVENLAFAXINE SUCCINATE:Joint swelling:0.22), (DOXAZOSIN MESYLATE:Joint swelling:0.38), (DOXEPIN HYDROCHLORIDE:Joint swelling:0.09), (EMPAGLIFLOZINLINAGLIPTIN:Joint swelling:0.18), (EVEROLIMUS:Joint swelling:0.54), (EVOLOCUMAB:Joint swelling:1.33), (FORMOTEROL FUMARATE:Joint swelling:0.06), (GLYCOPYRRONIUMINDACATEROL:Joint swelling:0.12), (HYDROCHLOROTHIAZIDEIRBESARTAN:Joint swelling:0.13), (HYDROXYCHLOROQUINE DIPHOSPHATE:Joint swelling:0.43), (HYDROXYZINE PAMOATE:Joint swelling:0.64), (HYOSCYAMINE:Joint swelling:0.68), (ILOPROST:Joint swelling:0.33), (INSULIN GLULISINE:Joint swelling:0.01), (IPRATROPIUM:Joint swelling:0.62), (LACTASE:Joint swelling:0.03), (LEDIPASVIRSOFOSBUVIR:Joint swelling:0.32), (LERCANIDIPINE:Joint swelling:0.46), (LISDEXAMFETAMINE DIMESYLATE:Joint swelling:0.51), (LOMITAPIDE MESYLATE:Joint swelling:0.07), (METHENAMINE HIPPURATE:Joint swelling:0.24), (MIDODRINE:Joint swelling:0.1 1), (NALOXONE HYDROCHLORIDEOXYCODONE HYDROCHLORIDE:Joint swelling:0.3), (NEBIVOLOL:Joint swelling:0.16), (NILOTINIB:Joint swelling:0.5), (OXCARBAZEPINE:Joint swelling:1.21), (PANTOPRAZOLE MAGNESIUM:Joint swelling:1.22), (PAROXETINE:Joint swelling:0.15), (PAROXETINEPAROXETINE HYDROCHLORIDE:Joint swelling:0.1), (PATIROMER:Joint swelling:0.01), (PAZOPANIB HYDROCHLORIDE:Joint swelling:0.1), (PEGINTERFERON BETA-1A:Joint swelling:0.27), (PENICILLIN:Joint swelling:0.23), (PREDNISOLONE ACETATE:Joint swelling:0.05), (PYRAZINAMIDE:Joint swelling:0.65), (RISEDRONATE SODIUM:Joint swelling:0.43), (RIZATRIPTAN BENZOATE:Joint swelling:0.31), (SCOPOLAMINE:Joint swelling:0.01), (SECUKINUMAB:Joint swelling:0.47), (SODIUM BICARBONATE:Joint swelling:0.14), (SUMATRIPTANSUMATRIPTAN SUCCINATE:Joint swelling:0.39), (TELMISARTAN:Joint swelling:0.69), (TETRACYCLINE:Joint swelling:0.67), (THALIDOMIDE:Joint swelling:0.25), (ALBUTEROL SULFATEIPRATROPIUM BROMIDE:Juvenile idiopathic arthritis:0.16), (ANASTROZOLE:Juvenile idiopathic arthritis:0.04),(ARMODAFINIL:Juvenile idiopathic arthritis:0.28), (BISOPROLOL FUMARATE:Juvenile idiopathic arthritis:0.17), (BUDESONIDEFORMOTEROL FUMARATE:Juvenile idiopathic arthritis:0), (BUMETANIDE:Juvenile idiopathic arthritis:0.37), (DACLIZUMAB:Juvenile idiopathic arthritis:0.02), (DILTIAZEM:Juvenile idiopathic arthritis:0.39), (DILTIAZEM HYDROCHLORIDE:Juvenile idiopathic arthritis:0.64), (ELETRIPTAN HYDROBROMIDE:Juvenile idiopathic arthritis:0.31), (ENALAPRIL MALEATE:Juvenile idiopathic arthritis:0.17), (ESTROGENS, CONJUGATEDMEDROXYPROGESTERONE ACETATE:Juvenile idiopathic arthritis:0.01), (ETHAMBUTOL HYDROCHLORIDE:Juvenile idiopathic arthritis:0.92), (EVOLOCUMAB:Juvenile idiopathic arthritis:0.43), (IBRUTINIB:Juvenile idiopathic arthritis:0.74), (LABETALOLLABETALOL HYDROCHLORIDE:Juvenile idiopathic arthritis:0.19), (LACOSAMIDE:Juvenile idiopathic arthritis:0.18), (LITHIUM CARBONATE:Juvenile idiopathic arthritis:0.11), (LOVASTATIN:Juvenile idiopathic arthritis:0.1), (METRONIDAZOLEMETRONIDAZOLE HYDROCHLORIDE:Juvenile idiopathic arthritis:0.5), (OLANZAPINE:Juvenile idiopathic arthritis:0.8), (ONABOTULINUMTOXINA:Juvenile idiopathic arthritis:0.21), (OXYGEN:Juvenile idiopathic arthritis:0.06), (PAROXETINE:Juvenile idiopathic arthritis:0.08), (PHENYTOIN:Juvenile idiopathic arthritis:0.44), (PRAMIPEXOLE DIHYDROCHLORIDE:Juvenile idiopathic arthritis:0.21), (PRAVASTATIN SODIUM:Juvenile idiopathic arthritis:0.3), (PRIMIDONE:Juvenile idiopathic arthritis:0.41), (RABEPRAZOLE SODIUM:Juvenile idiopathic arthritis:0.39), (ROPINIROLE HYDROCHLORIDE:Juvenile idiopathic arthritis:0), (SODIUM OXYBATE:Juvenile idiopathic arthritis:0.38), (SUMATRIPTANSUMATRIPTAN SUCCINATE:Juvenile idiopathic arthritis:0.35), (TERIFLUNOMIDE:Juvenile idiopathic arthritis:0.01), (TICARCILLIN:Juvenile idiopathic arthritis:0.13), (TIZANIDINE HYDROCHLORIDE:Juvenile idiopathic arthritis:0.5), (UMECLIDINIUM BROMIDEVILANTEROL TRIFENATATE:Juvenile idiopathic arthritis:0.11), (VANCOMYCIN HYDROCHLORIDE:Juvenile idiopathic arthritis:0), (VARENICLINE TARTRATE:Juvenile idiopathic arthritis:0.48), (ZOLPIDEMZOLPIDEM TARTRATE:Juvenile idiopathic arthritis:0.48), (PHENYTOIN:Kidney angiomyolipoma:0.07), (AMPHETAMINE ASPARTATEAMPHETAMINE SULFATEDEXTROAMPHETAMINE SACCHARATEDEXTROAMPHETAMINE SULFATE:Kidney infection:0.01), (BUMETANIDE:Kidney infection:0.19), (BUPROPION HYDROCHLORIDE:Kidney infection:0.56), (CLINDAMYCINCLINDAMYCIN PHOSPHATE:Kidney infection:0.07), (DILTIAZEM HYDROCHLORIDE:Kidney infection:0.03), (DIVALPROEX SODIUM:Kidney infection:0.11), (EVOLOCUMAB:Kidney infection:0.17), (RAMIPRIL:Kidney infection:0.27), (SACUBITRILVALSARTAN:Kidney infection:0.07), (SELEXIPAG:Kidney infection:0.17), (TERIFLUNOMIDE:Kidney infection:0.68), (TOPIRAMATE:Kidney infection:0.63), (VERAPAMIL HYDROCHLORIDE:Kidney infection:0.2), (FOLIC ACID:Klebsiella infection:0.02), (POSACONAZOLE:Klebsiella infection:0.06), (AMPHETAMINE ASPARTATEAMPHETAMINE SULFATEDEXTROAMPHETAMINE SACCHARATEDEXTROAMPHETAMINE SULFATE:Knee arthroplasty:0.17), (ATROPINE SULFATEDIPHENOXYLATE HYDROCHLORIDE:Knee arthroplasty:0.17), (BUMETANIDE:Knee arthroplasty:0.42), (BUSPIRONE HYDROCHLORIDE:Knee arthroplasty:0.06), (CALCIUM CARBONATECHOLECALCIFEROL:Knee arthroplasty:0.82), (CANDESARTAN:Knee arthroplasty:0.66), (CANDESARTAN CILEXETILHYDROCHLOROTHIAZIDE:Knee arthroplasty:0.31), (CERTOLIZUMAB PEGOL:Knee arthroplasty:0.55), (CHOLESTYRAMINE:Knee arthroplasty:0.09), (CLOTRIMAZOLE:Knee arthroplasty:0.27), (COLCHICINE:Knee arthroplasty:0.17), (CORTISONEHYDROCORTISONE:Knee arthroplasty:0.19), (DASATINIB:Knee arthroplasty:0.06), (DUTASTERIDE:Knee arthroplasty:0.14), (ESZOPICLONE:Knee arthroplasty:0.17), (IBRUTINIB:Knee arthroplasty:1.19), (INSULIN HUMAN:Knee arthroplasty:0.43), (LABETALOLLABETALOL HYDROCHLORIDE:Knee arthroplasty:0.25), (LINAGLIPTIN:Knee arthroplasty:0.29), (MELATONIN:Knee arthroplasty:0.27), (METHYLPREDNISOLONE ACETATE:Knee arthroplasty:0.59), (METHYLPREDNISOLONE SODIUM SUCCINATE:Knee arthroplasty:0.2), (METRONIDAZOLE:Knee arthroplasty:0.78), (METRONIDAZOLEMETRONIDAZOLE HYDROCHLORIDE:Knee arthroplasty:0.09), (MOXIFLOXACIN HYDROCHLORIDE:Knee arthroplasty:0.07), (NALOXONE HYDROCHLORIDEOXYCODONE HYDROCHLORIDE:Knee arthroplasty:0.04), (NEBIVOLOL HYDROCHLORIDE:Knee arthroplasty:0.16), (PENICILLIN:Knee arthroplasty:0.03), (PHENOBARBITAL:Knee arthroplasty:0.03), (PRIMIDONE:Knee arthroplasty:0.36), (QUETIAPINE FUMARATE:Knee arthroplasty:0.11), (RALOXIFENE HYDROCHLORIDE:Knee arthroplasty:0.58), (RANOLAZINE:Knee arthroplasty:0.67), (RISEDRONATE SODIUM:Knee arthroplasty:0.18), (SACUBITRILVALSARTAN:Knee arthroplasty:0.03), (SODIUM OXYBATE:Knee arthroplasty:0.07), (TETRACYCLINE:Knee arthroplasty:0.51), (TOCILIZUMAB:Knee arthroplasty:0.21), (TRIAMCINOLONE ACETONIDE:Knee arthroplasty:0.15), (VARENICLINE TARTRATE:Knee arthroplasty:0.1), (UNSPECIFIED INGREDIENT:Knee operation:0.3), (ACETAMINOPHENCODEINE PHOSPHATE:Lacrimation increased:0.18), (AMIODARONE:Lacrimation increased:0.22), (BISOPROLOL FUMARATE:Lacrimation increased:0.08), (CALCIUM CARBONATECHOLECALCIFEROL:Lacrimation increased:0.22), (CERTOLIZUMAB PEGOL:Lacrimation increased:0.28), (CHOLESTYRAMINE:Lacrimation increased:0.08), (CLINDAMYCINCLINDAMYCIN PHOSPHATE:Lacrimation increased:0.15), (CYCLOBENZAPRINE:Lacrimation increased:0.14), (DULOXETINE:Lacrimation increased:0.12), (ESZOPICLONE:Lacrimation increased:0.01), (EVOLOCUMAB:Lacrimation increased:0.38), (FERROUS SULFATEFERROUS SULFATE, DRIED:Lacrimation increased:0.19), (METHYLPREDNISOLONE SODIUM SUCCINATE:Lacrimation increased:0.29), (OXYGEN:Lacrimation increased:0.27), (PROMETHAZINEPROMETHAZINE HYDROCHLORIDE:Lacrimation increased:0.09), (SACUBITRILVALSARTAN:Lacrimation increased:0.35), (TERIFLUNOMIDE:Lacrimation increased:0.22), (ZOLPIDEMZOLPIDEM TARTRATE:Lacrimation increased:0.13), (BUPROPION HYDROCHLORIDE:Lactose intolerance:0.21), (REMIFENTANIL:Laryngeal oedema:0.05), (RAMIPRIL:Latent tuberculosis:0.03), (ALBUTEROL SULFATEIPRATROPIUM BROMIDE:Laxative supportive care:0.84), (ALCOHOL:Laxative supportive care:0.12),(ARMODAFINIL:Laxative supportive care:0.72), (ATROPINE SULFATEDIPHENOXYLATE HYDROCHLORIDE:Laxative supportive care:0.32), (BENAZEPRILBENAZEPRIL HYDROCHLORIDE:Laxative supportive care:0.01), (BENZONATATE:Laxative supportive care:0.21), (BROMAZEPAM:Laxative supportive care:0.27), (BUPRENORPHINE HYDROCHLORIDENALOXONE HYDROCHLORIDE:Laxative supportive care:0.45), (CALCIUM CARBONATEVITAMIN D:Laxative supportive care:0.34), (CANDESARTAN CILEXETIL:Laxative supportive care:0.67), (CIMETIDINE:Laxative supportive care:0.35), (CLARITHROMYCIN:Laxative supportive care:0.06), (CLINDAMYCIN:Laxative supportive care:0.06), (CORTISONEHYDROCORTISONE:Laxative supportive care:0.37), (DASATINIB:Laxative supportive care:0.79), (DESLORATADINE:Laxative supportive care:0.22), (DEVICE:Laxative supportive care:0.04), (DOFETILIDE:Laxative supportive care:0.28), (DOXAZOSIN MESYLATE:Laxative supportive care:0.37), (DOXEPIN:Laxative supportive care:0.03), (EDOXABAN TOSYLATE:Laxative supportive care:0.19), (ELETRIPTAN HYDROBROMIDE:Laxative supportive care:0.47), (ENALAPRIL MALEATE:Laxative supportive care:0.68), (EPLERENONE:Laxative supportive care:0), (EVOLOCUMAB:Laxative supportive care:0.67), (FINGOLIMOD HYDROCHLORIDE:Laxative supportive care:0.57), (FLUOXETINEFLUOXETINE HYDROCHLORIDE:Laxative supportive care:0.48), (FLUTICASONE:Laxative supportive care:0.5), (FORMOTEROL FUMARATE:Laxative supportive care:0.04), (GLIMEPIRIDE:Laxative supportive care:0.59), (HYDROCHLOROTHIAZIDEIRBESARTAN:Laxative supportive care:0.2), (HYDROXYCHLOROQUINE DIPHOSPHATE:Laxative supportive care:0), (IDELALISIB:Laxative supportive care:0.01), (ILOPROST:Laxative supportive care:0.34), (INDAPAMIDE:Laxative supportive care:0.28), (INDAPAMIDEPERINDOPRIL:Laxative supportive care:0.33), (INFLUENZA VIRUS VACCINE:Laxative supportive care:0.37), (IRBESARTAN:Laxative supportive care:0.69), (LERCANIDIPINE:Laxative supportive care:0.87), (LERCANIDIPINE HYDROCHLORIDE:Laxative supportive care:0.23), (LEVALBUTEROL HYDROCHLORIDE:Laxative supportive care:0.76), (LIDOCAINEPRILOCAINE:Laxative supportive care:0.51), (METAXALONE:Laxative supportive care:0.76), (METHENAMINE HIPPURATE:Laxative supportive care:0.43), (METOLAZONE:Laxative supportive care:0), (MODAFINIL:Laxative supportive care:0.43), (MYCOPHENOLATE MOFETILMYCOPHENOLATE MOFETIL HYDROCHLORIDE:Laxative supportive care:0.38), (NEBIVOLOL HYDROCHLORIDE:Laxative supportive care:0.56), (OMEGA-3-ACID ETHYL ESTERS:Laxative supportive care:0.63), (OXAZEPAM:Laxative supportive care:0.25), (PANTOPRAZOLE MAGNESIUM:Laxative supportive care:0.66), (PAROXETINEPAROXETINE HYDROCHLORIDE:Laxative supportive care:0.39), (PEGINTERFERON BETA-1A:Laxative supportive care:0.3), (PENICILLIN:Laxative supportive care:0.32), (PHENOBARBITAL:Laxative supportive care:0.57), (PHYTONADIONE:Laxative supportive care:0.06), (PIRFENIDONE:Laxative supportive care:0.05), (PONATINIB:Laxative supportive care:0.39), (RABEPRAZOLE:Laxative supportive care:0.28), (RIZATRIPTAN BENZOATE:Laxative supportive care:0.39), (SECUKINUMAB:Laxative supportive care:0.35), (SUMATRIPTANSUMATRIPTAN SUCCINATE:Laxative supportive care:0.24), (TELMISARTAN:Laxative supportive care:0.75), (THALIDOMIDE:Laxative supportive care:0.41), (TIMOLOL:Laxative supportive care:0.06), (TIOTROPIUM:Laxative supportive care:0.22), (UMECLIDINIUM BROMIDEVILANTEROL TRIFENATATE:Laxative supportive care:0.43), (VILAZODONE HYDROCHLORIDE:Laxative supportive care:0.22), (ZONISAMIDE:Laxative supportive care:0.15), (IBRUTINIB:Left ventricular dysfunction:0), (ILOPROST:Left ventricular dysfunction:0.28), (SELEXIPAG:Left ventricular dysfunction:0.02), (DILTIAZEM HYDROCHLORIDE:Left ventricular failure:0.13), (ILOPROST:Left ventricular failure:0.29), (ADO-TRASTUZUMAB EMTANSINE:Leukaemia:0.15), (ALENDRONIC ACID:Leukaemia:0.18), (BISACODYL OR DOCUSATE SODIUM:Leukaemia:0.36), (BUDESONIDE:Leukaemia:1.35), (CALCIUM ACETATE:Leukaemia:0.28), (CANDESARTAN:Leukaemia:0.72), (CANDESARTAN CILEXETIL:Leukaemia:0.58), (CANDESARTAN CILEXETILHYDROCHLOROTHIAZIDE:Leukaemia:0.27), (CERTOLIZUMAB PEGOL:Leukaemia:0.15), (CHOLESTYRAMINE:Leukaemia:0.34), (CLOTRIMAZOLE:Leukae mia:0.37), (CORTISONEHYDROCORTISONE:Leukaemia:0.08), (CYCLOBENZAPRINE HYDROCHLORIDE:Leukaemia:0.32), (CYCLOPHOSPHAMIDECYTARABINEDEXAMET HASONEDOXORUBICINMETHOTREXATEVINCRISTINE:Leukaemia:0.22), (DALFAMP RIDINE:Leukaemia:0.07), (DEVICE:Leukaemia:0.13), (DIVALPROEX SODIUM:Leukaemia:0.14), (ENALAPRIL MALEATE:Leukaemia:0.02), (EPINEPHRINE:Leukaemia:0.52), (EVOLOCUMAB:Leukaemia: 0.66), (FESOTERODINE FUMARATE:Leukaemia:0), (FLUTICASONE FUROATEVILANTEROL TRIFENATATE:Leukaemia:0.11), (FULVESTRANT:Leukaemia:0.19), (HYDROXYCHLORO QUINE DIPHOSPHATE:Leukaemia:0), (HYDROXYCHLOROQUINE SULFATE:Leukaemia:0.09), (IBRITUMOMAB TIUXETAN:Leukaemia:0.96), (ILOPROST:Leukaemia:0.19), (IMIPENEM:Leukaemia:0.09), (IT RACONAZOLE:Leukaemia:0.03), (LEDIPASVIRSOFOSBUVIR:Leukaemia:0.38), (LEVALB UTEROL HYDROCHLORIDE:Leukaemia:0.05), (METHADONE HYDROCHLORIDE:Leukaemia:0.06), (METHYLPHENIDATE HYDROCHLORIDE:Leukaemia:0.04), (MOXIFLOXACIN HYDROCHLORIDE:Leukaemia:0.07), (NITROFURANTOIN:Leukaemia:0.53), (NORTRIPTY LINE:Leukaemia:0.02), (OMEGA-3-ACID ETHYL ESTERS:Leukaemia:0.18), (OXCARBAZEPINE:Leukaemia:0.22), (PENTOSTATIN:Leukaemia:0.38), (PRAMIPEXOLE DIHYDROCHLORIDE:Leukaemia:0.1), (QUETIAPINE FUMARATE:Leukaemia:0.01), (RISEDRONATE SODIUM:Leukaemia:0.48), (SELEXIPAG:Leukaemia:0.52), (SULFAMETHOXAZOLE:Leukae mia:0.39), (THIOGUANINE:Leukaemia:0.22), (TIZANIDINE:Leukaemia:0.11), (ANASTROZO LE:Leukopenia:0.12), (THALIDOMIDE:Leukopenia:0.47), (DIVALPROEX SODIUM:Libido decreased:0.04), (VARENICLINE TARTRATE:Libido decreased:0.01), (AMIODARONE:Limb discomfort:0.44), (AMPHETAMINE ASPARTATEAMPHETAMINE SULFATEDEXTROAMPHETAMINE SACCHARATEDEXTROAMPHETAMINE SULFATE:Limb discomfort:0.65), (ATROPINE SULFATEDIPHENOXYLATE HYDROCHLORIDE:Limb discomfort:0.13), (BENAZEPRIL HYDROCHLORIDE:Limb discomfort:0.04), (BISACODYL OR DOCUSATE SODIUM:Limb discomfort:0.3), (BISOPROLOL FUMARATE:Limb discomfort:0.78), (BUMETANIDE:Limb discomfort:0.53), (CANDESARTAN:Limb discomfort:0.16), (CANDESARTAN CILEXETIL:Limb discomfort:0.09), (CARVEDILOL PHOSPHATE:Limb discomfort:0.15), (CLINDAMYCINCLINDAMYCIN PHOSPHATE:Limb discomfort:0.17), (CLOTRIMAZOLE:Limb discomfort:0.06), (DEVICE:Limb discomfort:0.94), (ENOXAPARIN:Limb discomfort:0.42), (EPINEPHRINE:Limb discomfort:0.14), (EVOLOCUMAB:Limb discomfort:1.18), (FESOTERODINE FUMARATE:Limb discomfort:0.02), (HYDROXYCHLOROQUINE DIPHOSPHATE:Limb discomfort:0.02), (HYDROXYZINE PAMOATE:Limb discomfort:0.47), (HYOSCYAMINE:Limb discomfort:0.25), (IBRUTINIB:Limb discomfort:0.58), (ILOPROST:Limb discomfort:0.46), (LABETALOLLABETALOL HYDROCHLORIDE:Limb discomfort:0.22), (LENALIDOMIDE:Limb discomfort:0.04), (LINAGLIPTIN:Limb discomfort:0.2), (METRONIDAZOLE:Limb discomfort:0.88), (OMEGA-3-ACID ETHYL ESTERS:Limb discomfort:0.45), (OXYBUTYNIN CHLORIDE:Limb discomfort:0.03), (PANTOPRAZOLE MAGNESIUM:Limb discomfort:0.02), (PAROXETINE:Limb discomfort:0.21), (POLYETHYLENE GLYCOLS:Limb discomfort:0.3), (RIOCIGUAT:Limb discomfort:0.81), (RISEDRONATE SODIUM:Limb discomfort:0.23), (SUMATRIPTANSUMATRIPTAN SUCCINATE:Limb discomfort:0.07), (TELMISARTAN:Limb discomfort:0.14), (TETRACYCLINE:Limb discomfort:0.29), (TOCILIZUMAB:Limb discomfort:0.26), (TRIAMTERENE:Limb discomfort:0.01), (FLUTICASONE PROPIONATE:Limb injury:0.11), (AMIODARONE:Lipid metabolism disorder:0.67), (CALCIUM CARBONATECHOLECALCIFEROL:Lipid metabolism disorder:0.07), (CLONAZEPAM:Lipid metabolism disorder:0.03), (DEVICE:Lipid metabolism disorder:0.26), (DEXLANSOPRAZOLE:Lipid metabolism disorder:0.31), (DIGOXIN:Lipid metabolism disorder:0.81), (DILTIAZEM HYDROCHLORIDE:Lipid metabolism disorder:0.41), (FERROUS SULFATEFERROUS SULFATE, DRIED:Lipid metabolism disorder:0.06), (GLIPIZIDE:Lipid metabolism disorder:0.07), (METOPROLOL TARTRATE:Lipid metabolism disorder:0.09), (NAPROXEN:Lipid metabolism disorder:0.6), (OCTREOTIDE ACETATE:Lipid metabolism disorder:0.2), (ACETAMINOPHENCODEINE PHOSPHATE:Lipidosis:0.31), (CERTOLIZUMAB PEGOL:Lipidosis:0.1), (DEVICE:Lipidosis:0.01), (DILTIAZEM HYDROCHLORIDE:Lipidosis:0.26), (ELETRIPTAN HYDROBROMIDE:Lipidosis:0.04), (ENOXAPARIN:Lipidosis:0.16), (ESZOPICLONE:Lipidosi s:0.27), (EVOLOCUMAB:Lipidosis:0.33), (IBRUTINIB:Lipidosis:0.56), (INSULIN ASPART:Lipidosis:0.15), (METAXALONE:Lipidosis:0.2), (MORPHINE:Lipidosis:0), (PHENO BARBITAL:Lipidosis:0.36), (SODIUM OXYBATE:Lipidosis:0.61), (TERIFLUNOMIDE:Lipidosis:0.22), (BUMETANIDE:Lipids:0.32), (DEXLANSOPRAZOLE:Lipids:0.07), (DILTIAZEM HYDROCHLORIDE:Lipids:0.56), (EMPAGLIFLOZINLINAGLIPTIN:Lipids:0.15), (FERROU S SULFATEFERROUS SULFATE, DRIED:Lipids:0.04), (FINGOLIMOD HYDROCHLORIDE:Lipids:0.18), (IBRUTINIB:Lipids:0.06), (ISOSORBIDE MONONITRATE:Lipids:0.22), (PANTOPRAZOLE:Lipids:0.51), (POTASSIUM:Lipids:0.51), (P RAVASTATIN SODIUM:Lipids:0.31), (RANITIDINE HYDROCHLORIDE:Lipids:0.22), (SACUBITRILVALSARTAN:Lipids:0.29), (TERIFLUNOM IDE:Lipids:0.37), (WARFARIN:Lipids:1.2), (ACETAMINOPHENCODEINE PHOSPHATE:Lipids abnormal:0.35), (CALCIUMVITAMIN D:Lipids abnormal:0.06), (CANAGLIFLOZINMETFORMIN HYDROCHLORIDE:Lipids abnormal:0.21), (CANDESARTAN:Lipids abnormal:1.07), (CANDESARTAN CILEXETILHYDROCHLOROTHIAZIDE:Lipids abnormal:0.14), (DASATINIB:Lipids abnormal:0.03), (DEXLANSOPRAZOLE:Lipids abnormal:0.63), (DICLOFENAC:Lipids abnormal:0.11), (EDOXABAN TOSYLATE:Lipids abnormal:0.14), (EMPAGLIFLOZINLINAGLIPTIN:Lipids abnormal:0.35), (EVEROLIMUS:Lipids abnormal:0.55), (HYDROCHLOROTHIAZIDELISINOPRIL:Lipids abnormal:0.23), (IBRUTINIB:Lipids abnormal:0.4), (INSULIN GLULISINE:Lipids abnormal:0.22), (LERCANIDIPINE:Lipids abnormal:0.39), (MELATONIN:Lipids abnormal:0.06), (NEBIVOLOL:Lipids abnormal:0.51), (NILOTINIB:Lipids abnormal:0.61), (OXYGEN:Lipids abnormal:0.52), (PANTOPRAZOLE MAGNESIUM:Lipids abnormal:0.06), (PAZOPANIB HYDROCHLORIDE:Lipids abnormal:0.05), (POLYETHYLENE GLYCOLS:Lipids abnormal:0.11), (QUETIAPINE:Lipids abnormal:0.22), (TERIFLUNOMIDE:Lipids abnormal:0.17), (VITAMIN B:Lipids abnormal:0.08), (ACETAMINOPHENCODEINE PHOSPHATE:Lipids increased:0.52), (ACYCLOVIR:Lipids increased:0.07), (ALBUTEROL SULFATEIPRATROPIUM BROMIDE:Lipids increased:0.34), (ALFACALCIDOL:Lipids increased:0.21), (AMPHETAMINE ASPARTATEAMPHETAMINE SULFATEDEXTROAMPHETAMINE SACCHARATEDEXTROAMPHETAMINE SULFATE:Lipids increased:0.13), (BISACODYL OR DOCUSATE SODIUM:Lipids increased:0.16), (BUSPIRONE HYDROCHLORIDE:Lipids increased:0.23), (CALCIUM ACETATE:Lipids increased:0.39), (CANAGLIFLOZINMETFORMIN HYDROCHLORIDE:Lipids increased:0.03), (CANDESARTAN CILEXETILHYDROCHLOROTHIAZIDE:Lipids increased:0.35), (CLINDAMYCINCLINDAMYCIN PHOSPHATE:Lipids increased:0.45), (CLOTRIMAZOLE:Lipids increased:0.04), (DOCUSATE:Lipids increased:0.21), (DOFETILIDE:Lipids increased:0), (DULAGLUTIDE:Lipids increased:0.42), (EMPAGLIFLOZINLINAGLIPTIN:Lipids increased:0.22), (EPINEPHRINE:Lipids increased:0.18), (EPLERENONE:Lipids increased:0.16), (ETIMICIN SULFATE:Lipids increased:0.09), (FESOTERODINE FUMARATE:Lipids increased:0.15), (FLECAINIDE:Lipids increased:0.2), (FLUTICASONE FUROATEVILANTEROL TRIFENATATE:Lipids increased:0.08), (HUMAN HEPATITIS B VIRUS IMMUNE GLOBULIN:Lipids increased:0.09), (HYDROXYZINE PAMOATE:Lipids increased:0.05), (IBRUTINIB:Lipids increased:1.04), (INDAPAMIDEPERINDOPRIL:Lipids increased:0), (INSULIN GLULISINE:Lipids increased:0.25), (IRON:Lipids increased:0.57), (LEDIPASVIRSOFOSBUVIR:Lipids increased:0.02), (LEUCOGEN:Lipids increased:0.08), (LITHIUM CARBONATE:Lipids increased:0.05), (MELATONIN:Lipids increased:0.28), (METHYLPREDNISOLONE SODIUM SUCCINATE:Lipids increased:1.13), (NAPROXEN:Lipids increased:1.13), (NITROFURANTOIN:Lipids increased:0.22), (OMEPRAZOLE MAGNESIUM:Lipids increased:0.59), (OXCARBAZEPINE:Lipids increased:0.66), (OXYGEN:Lipids increased:0.52), (PAROXETINEPAROXETINE HYDROCHLORIDE:Lipids increased:0.09), (PERINDOPRIL:Lipids increased:0.13), (PRAMIPEXOLE DIHYDROCHLORIDE:Lipids increased:0.02), (QUETIAPINE:Lipids increased:0.13), (RABEPRAZOLE:Lipids increased:0.01), (RALOXIFENE HYDROCHLORIDE:Lipids increased:0.09), (UMECLIDINIUM BROMIDEVILANTEROL TRIFENATATE:Lipids increased:0.25), (DEVICE:Lipodystrophy acquired:0.04), (ACETAMINOPHENOXYCODONEOXYCODONE HYDROCHLORIDE:Liver disorder:0.35), (AMISULPRIDE:Liver disorder:0.01), (AMLODIPINE BESYLATEATORVASTATIN CALCIUM:Liver disorder:0.03), (ASPIRIN LYSINE:Liver disorder:0.11), (AXITINIB:Liver disorder:0.15), (BENAZEPRILBENAZEPRIL HYDROCHLORIDE:Liver disorder:0.4), (BENDAMUSTINE HYDROCHLORIDE:Liver disorder:0.18), (BIFIDOBACTERIUM SPP.:Liver disorder:0.39), (BLINATUMOMAB:Liver disorder:0.21), (BUPRENORPHINE HYDROCHLORIDE:Liver disorder:0.12), (BUPROPION:Liver disorder:0.73), (CALCIUM CARBONATEVITAMIN D:Liver disorder:0.72), (CANAGLIFLOZINMETFORMIN HYDROCHLORIDE:Liver disorder:0.2), (CANAKINUMAB:Liver disorder:0.55), (CARISOPRODOL:Liver disorder:0.09), (CEFOTAXIME SODIUM:Liver disorder:0.07), (CLINDAMYCIN:Liver disorder:0.88), (CLOBETASOL:Liver disorder:0.09), (CLOZAPINE:Liver disorder:1.36), (DACLIZUMAB:Liver disorder:0.35), (DALFAMPRIDINE:Liver disorder:0.05), (DANAZOL:Liver disorder:0.82), (DEFERIPRONE:Liver disorder:0.16), (DESLORATADINE:Liver disorder:0.59), (DEXLANSOPRAZOLE:Liver disorder:1.36), (DIGITOXIN:Liver disorder:0.15), (DOFETILIDE:Liver disorder:0.23), (DOXYCYCLINE:Liver disorder:0.73), (DULAGLUTIDE:Liver disorder:0.03), (ELDECALCITOL:Liver disorder:0.06), (ELETRIPTAN HYDROBROMIDE:Liver disorder:0.67), (ELOTUZUMAB:Liver disorder:0.21), (EMPAGLIFLOZINLINAGLIPTIN:Liver disorder:0.09), (ENOXAPARIN:Liver disorder:1.43), (EPINASTINE HYDROCHLORIDE:Liver disorder:0.25), (ESTROGENS, CONJUGATEDMEDROXYPROGESTERONE ACETATE:Liver disorder:0.33), (EVOLOCUMAB:Liver disorder:1.18), (FENTANYL CITRATE:Liver disorder:0.2), (FESOTERODINE FUMARATE:Liver disorder:0.56), (FLECAINIDE:Liver disorder:0.45), (FLUDROCORTISONE ACETATE:Liver disorder:0.13), (FLUOCINONIDE:Liver disorder:0.19), (FUSIDIC ACID:Liver disorder:0.21), (GLYCOPYRRONIUMINDACATEROL:Liver disorder:0.28), (GRANULOCYTE COLONY-STIMULATING FACTOR NOS:Liver disorder:0.08), (HYDROCHLOROTHIAZIDEIRBESARTAN:Liver disorder:0.5), (HYDROXYCHLOROQUINE DIPHOSPHATE:Liver disorder:0.51), (HYDROXYZINE PAMOATE:Liver disorder:0.98), (IDELALISIB:Liver disorder:0.28), (ILOPROST:Liver disorder:0.37), (IMIPRAMINE HYDROCHLORIDE:Liver disorder:0.41), (IPRATROPIUM:Liver disorder:1.03), (IXAZOMIB:Liver disorder:0.5), (LABETALOLLABETALOL HYDROCHLORIDE:Liver disorder:0.74), (LACOSAMIDE:Liver disorder:0.19), (LAPATINIB DITOSYLATE:Liver disorder:0.5), (LERCANIDIPINE:Liver disorder:0.48), (LERCANIDIPINE HYDROCHLORIDE:Liver disorder:0.11), (LEUCOVORIN CALCIUM:Liver disorder:1.04), (LEVALBUTEROL HYDROCHLORIDE:Liver disorder:0.72), (LIDOCAINEPRILOCAINE:Liver disorder:0.45), (LINEZOLID:Liver disorder:0.08), (LOMITAPIDE MESYLATE:Liver disorder:0.14), (LOPERAMIDE:Liver disorder:0.29), (METHENAMINE HIPPURATE:Liver disorder:0.22), (METHENOLONE ENANTHATE:Liver disorder:0.69), (METHYLPHENIDATE HYDROCHLORIDE:Liver disorder:0.59), (METHYLPREDNISOLONE ACETATE:Liver disorder:0.51), (MODAFINIL:Liver disorder:0.32), (MORPHINE SULFATENALTREXONE HYDROCHLORIDE:Liver disorder:0.11), (MUPIROCIN:Liver disorder:0.17), (NABUMETONE:Liver disorder:0.19), (NALOXONE HYDROCHLORIDEOXYCODONE HYDROCHLORIDE:Liver disorder:0.44), (NATEGLINIDE:Liver disorder:0.07), (NEBIVOLOL:Liver disorder:0.29), (NEBIVOLOL HYDROCHLORIDE:Liver disorder:0.76), (NEVIRAPINE:Liver disorder:0.04), (NINTEDANIB:Liver disorder:0.56), (NIRAPARIB:Liver disorder:0.38), (OLODATEROL HYDROCHLORIDETIOTROPIUM BROMIDE MONOHYDRATE:Liver disorder:0.22), (PAMIDRONATE DISODIUM:Liver disorder:0.33), (PAROXETINEPAROXETINE HYDROCHLORIDE:Liver disorder:0.6), (PEGINTERFERON BETA-1A:Liver disorder:0.29), (PEMETREXED:Liver disorder:0.56), (PENTOSAN POLYSULFATE SODIUM:Liver disorder:0.03), (PLANTAGO SEED:Liver disorder:0.15), (POLYETHYLENE GLYCOLS:Liver disorder:1.39), (PRAMIPEXOLE:Liver disorder:0.28), (PRAMIPEXOLE DIHYDROCHLORIDE:Liver disorder:0.47), (RADIATION THERAPY:Liver disorder:0.04), (RAMUCIRUMAB:Liver disorder:0.55), (RIZATRIPTAN BENZOATE:Liver disorder:0.36), (ROFLUMILAST:Liver disorder:0.65), (SARPOGRELATE:Liver disorder:0.03), (SAXAGLIPTIN HYDROCHLORIDE:Liver disorder:0.02), (SEVELAMER CARBONATE:Liver disorder:0.06), (SODIUM FERROUS CITRATE:Liver disorder:0.06), (SODIUM PICOSULFATE:Liver disorder:0.01), (SOLIFENACIN SUCCINATE:Liver disorder:0.41), (SUMATRIPTANSUMATRIPTAN SUCCINATE:Liver disorder:0.5), (TERBUTALINE SULFATE:Liver disorder:0.25), (TERIFLUNOMIDE:Liver disorder:0.16), (TETRACYCLINE:Liver disorder:0.46), (THALIDOMIDE:Liver disorder:2.11), (TIMOLOL:Liver disorder:0.29), (TINZAPARIN SODIUM:Liver disorder:0.13), (TIOTROPIUM:Liver disorder:0.26), (TIPIRACIL HYDROCHLORIDETRIFLURIDINE:Liver disorder:0.03), (TOLTERODINE TARTRATE:Liver disorder:0.32), (TRIAMCINOLONE:Liver disorder:0.24), (UMECLIDINIUM BROMIDEVILANTEROL TRIFENATATE:Liver disorder:0.56), (VARENICLINE TARTRATE:Liver disorder:1.1), (ZOPICLONE:Liver disorder:0.59), (GADOVERSETAMIDE:Liver function test abnormal:0.03), (ACARBOSE:Liver transplant:0.17), (ACETAMINOPHENBUTALBITALCAFFEINE:Liver transplant:0.13), (AMINO ACIDSDEXTROSEELECTROLYTES NOS:Liver transplant:0), (ANASTROZOLE:Liver transplant:0.55), (ASUNAPREVIR:Liver transplant:0.27), (ATAZANAVIR:Liver transplant:0.35), (BENAZEPRIL HYDROCHLORIDE:Liver transplant:0.02), (BENAZEPRILBENAZEPRIL HYDROCHLORIDE:Liver transplant:0.04), (BUSPIRONE HYDROCHLORIDE:Liver transplant:0.16), (CANDESARTAN:Liver transplant:0.89), (CANDESARTAN CILEXETILHYDROCHLOROTHIAZIDE:Liver transplant:0.71), (CARFILZOMIB:Liver transplant:0.55), (CARVEDILOL PHOSPHATE:Liver transplant:0.16), (CERTOLIZUMAB PEGOL:Liver transplant:0.51), (CIPROFLOXACIN HYDROCHLORIDE:Liver transplant:0.54), (CLARITHROMYCIN:Liver transplant:0.11), (CLOZAPINE:Liver transplant:0.36), (CYCLOBENZAPRINE HYDROCHLORIDE:Liver transplant:0.16), (DALFAMPRIDINE:Liver transplant:0.09), (DALTEPARIN:Liver transplant:0.22), (DASATINIB:Liver transplant:0.83), (DILTIAZEM HYDROCHLORIDE:Liver transplant:2.04), (DIVALPROEX SODIUM:Liver transplant:0.86), (DULOXETINE:Liver transplant:0.38), (DUTASTERIDE:Liver transplant:0.41), (EDOXABAN TOSYLATE:Liver transplant:0.17), (ELTROMBOPAG OLAMINE:Liver transplant:0.04), (EPLERENONE:Liver transplant:0.06), (EVOLOCUMAB:Liver transplant:0.75), (EZETIMIBESIMVASTATIN:Liver transplant:0.09), (FLUTICASONE FUROATESALMETEROL XINAFOATE:Liver transplant:0.21), (GADOVERSETAMIDE:Liver transplant:0.28), (GLYCOPYRRONIUMINDACATEROL:Liver transplant:0), (IBRUTINIB:Liver transplant:0.88), (IDELALISIB:Liver transplant:0.2), (ILOPROST:Liver transplant:0.23), (IMATINIB MESYLATE:Liver transplant:0.88), (INDAPAMIDEPERINDOPRIL:Liver transplant:0.65), (INFLUENZA VIRUS VACCINE:Liver transplant:0.15), (IRON SUCROSE:Liver transplant:0.18), (ISOSORBIDE:Liver transplant:0.46), (LENALIDOMIDE:Liver transplant:0.78), (LIDOCAINEPRILOCAINE:Liver transplant:0.05), (MARAVIROC:Liver transplant:0.06), (MELOXICAM:Liver transplant:0.77), (MINOCYCLINEMINOCYCLINE HYDROCHLORIDE:Liver transplant:0.12), (MOXIFLOXACIN:Liver transplant:0.6), (NAPROXEN SODIUM:Liver transplant:0.03), (NILOTINIB:Liver transplant:0.82), (OMEPRAZOLE SODIUM:Liver transplant:0.03), (OSELTAMIVIR:Liver transplant:0.25), (PANTOPRAZOLE MAGNESIUM:Liver transplant:0.47), (PHENOBARBITAL:Liver transplant:0.91), (PHENPROCOUMON:Liver transplant:0.04), (PONATINIB:Liver transplant:0.57), (PREDNISOLONE SODIUM PHOSPHATE:Liver transplant:0.1), (PRIMIDONE:Liver transplant:0.62), (PYRIDOXINE:Liver transplant:0.21), (SACUBITRILVALSARTAN:Liver transplant:0.36), (SECUKINUMAB:Liver transplant:0.29), (TERIFLUNOMIDE:Liver transplant:0.23), (TIMOLOL:Liver transplant:0.45), (TIOTROPIUM:Liver transplant:0.23), (TOLTERODINE TARTRATE:Liver transplant:0.05), (UMECLIDINIUM BROMIDEVILANTEROL TRIFENATATE:Liver transplant:0), (ALENDRONIC ACID:Local anaesthesia:0), (BUMETANIDE:Local anaesthesia:0.44), (CALCITRIOL:Local anaesthesia:0.15), (CANDESARTAN CILEXETILHYDROCHLOROTHIAZIDE:Local anaesthesia:0.19), (CEPHALEXIN:Local anaesthesia:0.65), (CERTOLIZUMAB PEGOL:Local anaesthesia:0.84), (CHOLESTYRAMINE:Local anaesthesia:0.01), (CITALOPRAM HYDROBROMIDE:Local anaesthesia:1.02), (CLOZAPINE:Local anaesthesia:0.29), (CYCLOBENZAPRINE:Local anaesthesia:0.26), (CYCLOBENZAPRINE HYDROCHLORIDE:Local anaesthesia:0.04), (DALFAMPRIDINE:Local anaesthesia:0.17), (DASATINIB:Local anaesthesia:0.04), (DIVALPROEX SODIUM:Local anaesthesia:0.27), (DOXYCYCLINE:Local anaesthesia:0.07), (DUTASTERIDE:Local anaesthesia:0.11), (ENOXAPARIN:Local anaesthesia:0.92), (EVEROLIMUS:Local anaesthesia:0.37), (EVOLOCUMAB:Local anaesthesia:0.61), (FOSPHENYTOINFOSPHENYTOIN SODIUM:Local anaesthesia:0.37), (HYDROCODONE:Local anaesthesia:0.1), (IBRUTINIB:Local anaesthesia:0.95), (INDAPAMIDE:Local anaesthesia:0.12), (IRBESARTAN:Local anaesthesia:0.12), (IRON:Local anaesthesia:0.45), (LABETALOLLABETALOL HYDROCHLORIDE:Local anaesthesia:0.24), (LINAGLIPTIN:Local anaesthesia:0.15), (LOVASTATIN:Local anaesthesia:0.33), (MELOXICAM:Local anaesthesia:0.7), (METHADONE HYDROCHLORIDE:Local anaesthesia:0.11), (METHYLENE BLUE:Local anaesthesia:0.35), (OXCARBAZEPINE:Local anaesthesia:0.64), (PRIMIDONE:Local anaesthesia:0.11), (RANOLAZINE:Local anaesthesia:0.24), (SACUBITRILVALSARTAN:Local anaesthesia:0.34), (SODIUM OXYBATE:Local anaesthesia:0.02), (SOTALOL:Local anaesthesia:0.03), (SPIRONOLACTONE:Local anaesthesia:1.36), (TAMSULOSIN:Local anaesthesia:1.06), (TERIFLUNOMIDE:Local anaesthesia:0.34), (THALIDOMIDE:Local anaesthesia:0.49), (TICAGRELOR:Local anaesthesia:0.08), (TIZANIDINE HYDROCHLORIDE:Local anaesthesia:0.26), (TOCILIZUMAB:Local anaesthesia:0.14), (TOFACITINIB CITRATE:Local anaesthesia:0.18), (TOPIRAMATE:Local anaesthesia:0.87), (VALGANCICLOVIR HYDROCHLORIDE:Local anaesthesia:0.18), (EVOLOCUMAB:Local swelling:0.06), (ALBUTEROL SULFATEIPRATROPIUM BROMIDE:Localised infection:0.13), (AMPHETAMINE ASPARTATEAMPHETAMINE SULFATEDEXTROAMPHETAMINE SACCHARATEDEXTROAMPHETAMINE SULFATE:Localised infection:0.17), (ATROPINE SULFATEDIPHENOXYLATE HYDROCHLORIDE:Localised infection:0.07), (BISACODYL OR DOCUSATE SODIUM:Localised infection:0.21), (CALCIUM ACETATE:Localised infection:0.1), (CANDESARTAN CILEXETIL:Localised infection:0.23), (CEFTRIAXONE SODIUM:Localised infection:0.28), (CYCLOBENZAPRINE:Localised infection:0.45), (DESLORATADINE:Localised infection:0.08), (DICYCLOMINE HYDROCHLORIDE:Localised infection:0.18), (DIVALPROEX SODIUM:Localised infection:0.17), (ENOXAPARIN:Localised infection:0.71), (EVOLOCUMAB:Localised infection:0.64), (FENOFIBRATE:Localised infection:0.34), (FERROUS SULFATEFERROUS SULFATE, DRIED:Localised infection:0.81), (HEPARIN SODIUM:Localised infection:0.27), (HYDROXYCHLOROQUINE DIPHOSPHATE:Localised infection:0.1), (LERCANIDIPINE:Localised infection:0.04), (NEBIVOLOL:Localised infection:0.48), (NILOTINIB:Localised infection:0.11), (OXCARBAZEPINE:Localised infection:0.71), (OXYGEN:Localised infection:0.07), (PANTOPRAZOLE MAGNESIUM:Localised infection:0.1), (PAROXETINE:Localised infection:0.11), (PHENOBARBITAL:Localised infection:0.09), (POLYETHYLENE GLYCOL 3350:Localised infection:0.15), (POLYETHYLENE GLYCOLS:Localised infection:0.52), (PRAMIPEXOLE DIHYDROCHLORIDE:Localised infection:0.17), (RABEPRAZOLE SODIUM:Localised infection:0.04), (RANOLAZINE:Localised infection:0.59), (SACUBITRILVALSARTAN:Localised infection:0.23), (SECUKINUMAB:Localised infection:0.48), (SOTALOL:Localised infection:0.26), (TELMISARTAN:Localised infection:0.09), (TIZANIDINE HYDROCHLORIDE:Localised infection:0.26), (TOFACITINIB CITRATE:Localised infection:0.18), (VITAMIN B:Localised infection:0.29), (AMIODARONE HYDROCHLORIDE:Low density lipoprotein increased:0.34),(ARMODAFINIL:Low density lipoprotein increased:0.23), (BISACODYL OR DOCUSATE SODIUM:Low density lipoprotein increased:0.16), (CANDESARTAN CILEXETIL:Low density lipoprotein increased:0.47), (CANDESARTAN CILEXETILHYDROCHLOROTHIAZIDE:Low density lipoprotein increased:0.05), (CARVEDILOL PHOSPHATE:Low density lipoprotein increased:0.24), (DEVICE:Low density lipoprotein increased:0.55), (DEXLANSOPRAZOLE:Low density lipoprotein increased:1.5), (DICLOFENAC:Low density lipoprotein increased:0.17), (DULAGLUTIDE:Low density lipoprotein increased:0.14), (DULOXETINE:Low density lipoprotein increased:0.27), (ELETRIPTAN HYDROBROMIDE:Low density lipoprotein increased:0.14), (HYDROXYZINE PAMOATE:Low density lipoprotein increased:0.01), (IBRUTINIB:Low density lipoprotein increased:1.07), (LEVALBUTEROL HYDROCHLORIDE:Low density lipoprotein increased:0.32), (LIDOCAINE:Low density lipoprotein increased:0.25), (LISDEXAMFETAMINE DIMESYLATE:Low density lipoprotein increased:0.14), (METRONIDAZOLE:Low density lipoprotein increased:0.58), (NITROFURANTOIN:Low density lipoprotein increased:0.03), (OXYGEN:Low density lipoprotein increased:0.19), (PANTOPRAZOLE MAGNESIUM:Low density lipoprotein increased:0.34), (POLYETHYLENE GLYCOLS:Low density lipoprotein increased:0.08), (PRAMIPEXOLE DIHYDROCHLORIDE:Low density lipoprotein increased:0.13), (PRIMIDONE:Low density lipoprotein increased:0.12), (RISEDRONATE SODIUM:Low density lipoprotein increased:0.5), (RIZATRIPTAN BENZOATE:Low density lipoprotein increased:0.08), (SECUKINUMAB:Low density lipoprotein increased:0.02), (TERIFLUNOMIDE:Low density lipoprotein increased:1.31), (TETRACYCLINE:Low density lipoprotein increased:0.42), (THYROID, PORCINE:Low density lipoprotein increased:0.31), (TIZANIDINE HYDROCHLORIDE:Low density lipoprotein increased:0.21), (TOCILIZUMAB:Low density lipoprotein increased:0.01), (UMECLIDINIUM BROMIDEVILANTEROL TRIFENATATE:Low density lipoprotein increased:0.09), (VARENICLINE TARTRATE:Low density lipoprotein increased:0.29), (ACENOCOUMAROL:Lower respiratory tract infection:0.02), (ACETAMINOPHENBUTALBITALCAFFEINE:Lower respiratory tract infection:0.18), (ALCOHOL:Lower respiratory tract infection:0.45), (AMISULPRIDE:Lower respiratory tract infection:0.17),(ARMODAFINIL:Lower respiratory tract infection:0.05), (ATOVAQUONE:Lower respiratory tract infection:0.03), (BENAZEPRIL HYDROCHLORIDE:Lower respiratory tract infection:0.34), (BISACODYL OR DOCUSATE SODIUM:Lower respiratory tract infection:0.39), (BUPRENORPHINE HYDROCHLORIDE:Lower respiratory tract infection:0.32), (BUSPIRONE HYDROCHLORIDE:Lower respiratory tract infection:0.27), (CALCIUMVITAMIN D:Lower respiratory tract infection:0.81), (CANAKINUMAB:Lower respiratory tract infection:0.19), (CANDESARTAN CILEXETILHYDROCHLOROTHIAZIDE:Lower respiratory tract infection:0.94), (CILASTATIN SODIUMIMIPENEM:Lower respiratory tract infection:0.96), (COLISTIMETHATE SODIUM:Lower respiratory tract infection:0.39), (CYCLOBENZAPRINE:Lower respiratory tract infection:0.44), (DICYCLOMINE HYDROCHLORIDE:Lower respiratory tract infection:0.05), (DIMENHYDRINATE:Lower respiratory tract infection:0.1), (DIVALPROEX SODIUM:Lower respiratory tract infection:0.18), (DOFETILIDE:Lower respiratory tract infection:0.42), (DRONABINOL:Lower respiratory tract infection:0.38), (ESZOPICLONE:Lower respiratory tract infection:0.35), (EVOLOCUMAB:Lower respiratory tract infection:0.92), (FENOTEROL HYDROBROMIDEIPRATROPIUM BROMIDE:Lower respiratory tract infection:0.36), (FESOTERODINE FUMARATE:Lower respiratory tract infection:0.26), (FULVESTRANT:Lower respiratory tract infection:0.69), (GLIMEPIRIDE:Lower respiratory tract infection:0.24), (GLIPIZIDE:Lower respiratory tract infection:0.63), (GLYCOPYRRONIUMINDACATEROL:Lower respiratory tract infection:0.97), (HYDROCHLOROTHIAZIDEVALSARTAN:Lower respiratory tract infection:0.16), (HYDROMORPHONE HYDROCHLORIDE:Lower respiratory tract infection:0.23), (HYDROXYZINE PAMOATE:Lower respiratory tract infection:0.17), (ILOPROST:Lower respiratory tract infection:0.27), (IMATINIB MESYLATE:Lower respiratory tract infection:0.69), (LEDIPASVIRSOFOSBUVIR:Lower respiratory tract infection:1.11), (LERCANIDIPINE HYDROCHLORIDE:Lower respiratory tract infection:0.42), (LEVALBUTEROL:Lower respiratory tract infection:0), (LEVALBUTEROL HYDROCHLORIDE:Lower respiratory tract infection:0.58), (LISDEXAMFETAMINE DIMESYLATE:Lower respiratory tract infection:0.05), (LUMACAFTOR:Lower respiratory tract infection:0.43), (MELATONIN:Lower respiratory tract infection:0.51), (MEPOLIZUMAB:Lower respiratory tract infection:0.92), (MESNA:Lower respiratory tract infection:0.38), (METFORMIN HYDROCHLORIDEVILDAGLIPTIN:Lower respiratory tract infection:0.09), (METHYLPREDNISOLONE SODIUM SUCCINATE:Lower respiratory tract infection:1.22), (MICAFUNGIN:Lower respiratory tract infection:0.47), (MYCOPHENOLATE MOFETILMYCOPHENOLATE MOFETIL HYDROCHLORIDE:Lower respiratory tract infection:0.08), (NALOXONE HYDROCHLORIDEOXYCODONE HYDROCHLORIDE:Lower respiratory tract infection:0.65), (NEBIVOLOL HYDROCHLORIDE:Lower respiratory tract infection:0.24), (OLOPATADINE:Lower respiratory tract infection:0.06), (OXCARBAZEPINE:Lower respiratory tract infection:1.29), (PAROXETINE:Lower respiratory tract infection:0.55), (PEMETREXED DISODIUM:Lower respiratory tract infection:0.41), (PONATINIB:Lower respiratory tract infection:0.3), (PRIMIDONE:Lower respiratory tract infection:0.25), (PROBIOTICS NOS:Lower respiratory tract infection:0.32), (PROCHLORPERAZINE MALEATE:Lower respiratory tract infection:0.11), (PROMETHAZINE HYDROCHLORIDE:Lower respiratory tract infection:0.12), (ROFLUMILAST:Lower respiratory tract infection:0.64), (ROPINIROLE HYDROCHLORIDE:Lower respiratory tract infection:0.04), (SULFAMETHOXAZOLE:Lower respiratory tract infection:0.18), (THALIDOMIDE:Lower respiratory tract infection:1.18), (TIZANIDINE HYDROCHLORIDE:Lower respiratory tract infection:0.12), (TOFACITINIB CITRATE:Lower respiratory tract infection:0.31), (VALGANCICLOVIR HYDROCHLORIDE:Lower respiratory tract infection:0.34), (ZOLPIDEMZOLPIDEM TARTRATE:Lower respiratory tract infection:1.27), (CALCIUM CARBONATECHOLECALCIFEROL:Lumbar radiculopathy:0.18), (CERTOLIZUMAB PEGOL:Lumbar radiculopathy:0.19), (DALFAMPRIDINE:Lumbar radiculopathy:0.12), (EVOLOCUMAB:Lumbar radiculopathy:0.67), (FERROUS SULFATE:Lumbar radiculopathy:0.04), (IRON:Lumbar radiculopathy:0.12), (ISOSORBIDE MONONITRATE:Lumbar radiculopathy:0.07), (MAGNESIUM:Lumbar radiculopathy:0), (RAMIPRIL:Lumbar radiculopathy:0.4), (SODIUM OXYBATE:Lumbar radiculopathy:0.09), (TERIFLUNOMIDE:Lumbar radiculopathy:0.05), (ACARBOSE:Lumbar spinal stenosis:0.1), (ALBUTEROL SULFATEIPRATROPIUM BROMIDE:Lumbar spinal stenosis:0.18), (ALENDRONIC ACID:Lumbar spinal stenosis:0.01), (AMOXICILLIN:Lumbar spinal stenosis:0.4), (AMPHETAMINE ASPARTATEAMPHETAMINE SULFATEDEXTROAMPHETAMINE SACCHARATEDEXTROAMPHETAMINE SULFATE:Lumbar spinal stenosis:0.31), (ANASTROZOLE:Lumbar spinal stenosis:0.39), (BEPRIDIL:Lumbar spinal stenosis:0.31), (BUDESONIDE:Lumbar spinal stenosis:1), (CANAGLIFLOZIN:Lumbar spinal stenosis:0.04), (CANDESARTAN CILEXETILHYDROCHLOROTHIAZIDE:Lumbar spinal stenosis:0.26), (CETIRIZINE HYDROCHLORIDE:Lumbar spinal stenosis:0.44), (CHOLESTYRAMINE:Lumbar spinal stenosis:0.21), (CLINDAMYCINCLINDAMYCIN PHOSPHATE:Lumbar spinal stenosis:0.05), (CLOTRIMAZOLE:Lumbar spinal stenosis:0.04), (CYCLOBENZAPRINE:Lumbar spinal stenosis:0.21), (DALFAMPRIDINE:Lumbar spinal stenosis:0.02), (DAPAGLIFLOZIN PROPANEDIOL:Lumbar spinal stenosis:0.06), (DASATINIB:Lumbar spinal stenosis:0.35), (DEVICE:Lumbar spinal stenosis:0.39), (DEXLANSOPRAZOLE:Lumbar spinal stenosis:0.77), (DICLOFENAC:Lumbar spinal stenosis:0.56), (DILTIAZEM:Lumbar spinal stenosis:0.54), (EDOXABAN TOSYLATE:Lumbar spinal stenosis:0.37), (ENOXAPARIN:Lumbar spinal stenosis:0.49), (ENOXAPARIN SODIUM:Lumbar spinal stenosis:0.24), (EPINEPHRINE:Lumbar spinal stenosis:0.22), (EVOLOCUMAB:Lumbar spinal stenosis:0.52), (FLECAINIDE:Lumbar spinal stenosis:0.2), (GLIPIZIDE:Lumbar spinal stenosis:0.62), (IBRUTINIB:Lumbar spinal stenosis:1.29), (ISOSORBIDE:Lumbar spinal stenosis:0.22), (ISOSORBIDE MONONITRATE:Lumbar spinal stenosis:0.7), (METHYLPREDNISOLONE SODIUM SUCCINATE:Lumbar spinal stenosis:0.64), (NEBIVOLOL:Lumbar spinal stenosis:0.37), (NILOTINIB:Lumbar spinal stenosis:0.75), (NYSTATIN:Lumbar spinal stenosis:0.04), (OMEGA-3-ACID ETHYL ESTERS:Lumbar spinal stenosis:0.23), (OXCARBAZEPINE:Lumbar spinal stenosis:0.09), (PANTOPRAZOLE MAGNESIUM:Lumbar spinal stenosis:0.18), (POLYETHYLENE GLYCOLS:Lumbar spinal stenosis:0.36), (POTASSIUM:Lumbar spinal stenosis:0.97), (PRAMIPEXOLE DIHYDROCHLORIDE:Lumbar spinal stenosis:0.16), (PROMETHAZINEPROMETHAZINE HYDROCHLORIDE:Lumbar spinal stenosis:0.18), (RAMIPRIL:Lumbar spinal stenosis:1.26), (TEMAZEPAM:Lumbar spinal stenosis:0.27), (TERIFLUNOMIDE:Lumbar spinal stenosis:0.08), (TOCILIZUMAB:Lumbar spinal stenosis:0.12), (VARENICLINE TARTRATE:Lumbar spinal stenosis:0.48), (VITAMIN B:Lumbar spinal stenosis:0.1), (ABIRATERONE ACETATE:Lung adenocarcinoma:0.13), (ACETAMINOPHENBUTALBITALCAFFEINE:Lung adenocarcinoma:0.09), (ACYCLOVIR:Lung adenocarcinoma:0.22), (ADENINE:Lung adenocarcinoma:0.19), (ADO-TRASTUZUMAB EMTANSINE:Lung adenocarcinoma:1.19), (ALCOHOL:Lung adenocarcinoma:0.1), (ALDESLEUKIN:Lung adenocarcinoma:0.06), (ALENDRONIC ACID:Lung adenocarcinoma:0.21), (ALISKIREN:Lung adenocarcinoma:0.22), (AMISULPRIDE:Lung adenocarcinoma:0.06), (AMLODIPINEVALSARTAN:Lung adenocarcinoma:0.3), (AMOXAPINE:Lung adenocarcinoma:0.22), (BENAZEPRIL HYDROCHLORIDE:Lung adenocarcinoma:0.6), (BENAZEPRILBENAZEPRIL HYDROCHLORIDE:Lung adenocarcinoma:0.2), (BEPRIDIL:Lung adenocarcinoma:0.26), (BERAPROST SODIUM:Lung adenocarcinoma:0.03), (BILASTINE:Lung adenocarcinoma:0.3), (BUSPIRONE HYDROCHLORIDE:Lung adenocarcinoma:0.22), (CALCITONIN:Lung adenocarcinoma:0.05), (CALCITONIN SALMON:Lung adenocarcinoma:0.42), (CALCIUM ACETATE:Lung adenocarcinoma:0.05), (CALCIUM CITRATE:Lung adenocarcinoma:0.09), (CANAKINUMAB:Lung adenocarcinoma:0.01), (CAPTOPRIL:Lung adenocarcinoma:0.09), (CARFILZOMIB:Lung adenocarcinoma:0.26), (CARVEDILOL PHOSPHATE:Lung adenocarcinoma:0.3), (CEFAZOLIN SODIUM:Lung adenocarcinoma:0.04), (CEFTRIAXONE SODIUM:Lung adenocarcinoma:1.27), (CERTOLIZUMAB PEGOL:Lung adenocarcinoma:0.8), (CETUXIMAB:Lung adenocarcinoma:1.04), (CHOLESTYRAMINE:Lung adenocarcinoma:1.27), (CICLESONIDE:Lung adenocarcinoma:0.36), (CLINDAMYCINCLINDAMYCIN PHOSPHATE:Lung adenocarcinoma:1.48), (CLOBAZAM:Lung adenocarcinoma:0.33), (CLOZAPINE:Lung adenocarcinoma:0.75), (CORTISONEHYDROCORTISONE:Lung adenocarcinoma:0.58), (DALTEPARIN:Lung adenocarcinoma:0.74), (DASATINIB:Lung adenocarcinoma:1.49), (DEXAMETHASONE ACETATE:Lung adenocarcinoma:0.64), (DEXLANSOPRAZOLE:Lung adenocarcinoma:1.46), (DICYCLOMINE HYDROCHLORIDE:Lung adenocarcinoma:0.43), (DIGITOXIN:Lung adenocarcinoma:0.53), (DILTIAZEM:Lung adenocarcinoma:0.35), (DIPYRIDAMOLE:Lung adenocarcinoma:0.74), (DIVALPROEX SODIUM:Lung adenocarcinoma:0.96), (DOFETILIDE:Lung adenocarcinoma:0.06), (DULOXETINE:Lung adenocarcinoma:0.23), (EDOXABAN TOSILATE MONOHYDRATE:Lung adenocarcinoma:0.58), (ELETRIPTAN HYDROBROMIDE:Lung adenocarcinoma:0.06), (ERIBULIN:Lung adenocarcinoma:0.6), (ERIBULIN MESYLATE:Lung adenocarcinoma:0.04), (ERYTHROMYCIN:Lung adenocarcinoma:0.2), (ETRETINATE:Lung adenocarcinoma:0.78), (EXEMESTANE:Lung adenocarcinoma:0.76), (FERROUS FUMARATE:Lung adenocarcinoma:0.29), (FERROUS SULFATEFERROUS SULFATE, DRIED:Lung adenocarcinoma:2), (FERROUS SULFATEFOLIC ACID:Lung adenocarcinoma:0.19), (FESOTERODINE FUMARATE:Lung adenocarcinoma:0.51), (FLUDROCORTISONE ACETATE:Lung adenocarcinoma:0.27), (FLUTICASONE FUROATE:Lung adenocarcinoma:0.42), (FONDAPARINUX SODIUM:Lung adenocarcinoma:0.05), (FORMOTEROL FUMARATE:Lung adenocarcinoma:0.83), (FORMOTEROL FUMARATE DIHYDRATEMOMETASONE FUROATE:Lung adenocarcinoma:0.1), (FUSIDIC ACID:Lung adenocarcinoma:0.47), (GLIPIZIDE:Lung adenocarcinoma:0.87), (HYDRALAZINE HYDROCHLORIDE:Lung adenocarcinoma:0.51), (HYDROCHLOROTHIAZIDELISINOPRIL:Lung adenocarcinoma:0.53), (HYDROXYZINE PAMOATE:Lung adenocarcinoma:0.27), (IMIPRAMINE HYDROCHLORIDE:Lung adenocarcinoma:0.37), (INTERFERON ALFA-2B:Lung adenocarcinoma:0.02), (LABETALOL HYDROCHLORIDE:Lung adenocarcinoma:0.07), (LABETALOLLABETALOL HYDROCHLORIDE:Lung adenocarcinoma:0.38), (LAPATINIB DITOSYLATE:Lung adenocarcinoma:0.5), (LEDIPASVIRSOFOSBUVIR:Lung adenocarcinoma:0.33), (LEUCOVORIN CALCIUM:Lung adenocarcinoma:0.43), (LEVALBUTEROL HYDROCHLORIDE:Lung adenocarcinoma:0.35), (LITHIUM CARBONATE:Lung adenocarcinoma:0.22), (MEPOLIZUMAB:Lung adenocarcinoma:0.02), (METHENOLONE ENANTHATE:Lung adenocarcinoma:0), (METRONIDAZOLE:Lung adenocarcinoma:0.75), (MIGLITOL:Lung adenocarcinoma:0.36), (MINODRONIC ACID:Lung adenocarcinoma:0.18), (MOXIFLOXACIN HYDROCHLORIDE:Lung adenocarcinoma:1.42), (MYCOPHENOLATE MOFETILMYCOPHENOLATE MOFETIL HYDROCHLORIDE:Lung adenocarcinoma:0.05), (NILOTINIB:Lung adenocarcinoma:1.96), (PAMIDRONATE DISODIUM:Lung adenocarcinoma:0.32), (PANTOPRAZOLE MAGNESIUM:Lung adenocarcinoma:1.12), (PAZOPANIB HYDROCHLORIDE:Lung adenocarcinoma:1.08), (PERTUZUMAB:Lung adenocarcinoma:0.21), (PONATINIB:Lung adenocarcinoma:0.48), (POTASSIUM CITRATETRISODIUM CITRATE DIHYDRATE:Lung adenocarcinoma:0), (PREDNISOLONE ACETATE:Lung adenocarcinoma:0.03), (PRIMIDONE:Lung adenocarcinoma:0.85), (PRISTINAMYCIN:Lung adenocarcinoma:0.29), (PYRAZINAMIDE:Lung adenocarcinoma:0.64), (RANOLAZINE:Lung adenocarcinoma:0.71), (ROFLUMILAST:Lung adenocarcinoma:0.71), (ROPINIROLE HYDROCHLORIDE:Lung adenocarcinoma:0.11), (SACUBITRILVALSARTAN:Lung adenocarcinoma:0.05), (SECUKINUMAB:Lung adenocarcinoma:0.47), (TERBINAFINE HYDROCHLORIDE:Lung adenocarcinoma:0.29), (THALIDOMIDE:Lung adenocarcinoma:0.67), (TIPIRACIL HYDROCHLORIDETRIFLURIDINE:Lung adenocarcinoma:0.48), (TOPIRAMATE:Lung adenocarcinoma:1.77), (ZIV-AFLIBERCEPT:Lung adenocarcinoma:0.04), (AMIODARONE:Lung adenocarcinoma stage IV:0.14), (BUMETANIDE:Lung adenocarcinoma stage IV:0.16), (DEXLANSOPRAZOLE:Lung adenocarcinoma stage IV:0.31), (FERROUS SULFATE:Lung adenocarcinoma stage IV:0.07), (IBRUTINIB:Lung adenocarcinoma stage IV:0.07), (METRONIDAZOLE:Lung adenocarcinoma stage IV:0.54), (NILOTINIB:Lung adenocarcinoma stage IV:0.21), (PANTOPRAZOLE:Lung adenocarcinoma stage IV:0.41), (POTASSIUM:Lung adenocarcinoma stage IV:0.03), (RISEDRONATE SODIUM:Lung adenocarcinoma stage IV:0.03), (UNSPECIFIED INGREDIENT:Lung adenocarcinoma stage IV:0.74), (ADO-TRASTUZUMAB EMTANSINE:Lung cancer metastatic:0.05), (ANASTROZOLE:Lung cancer metastatic:0.54), (BUMETANIDE:Lung cancer metastatic:0.43), (BUPROPION HYDROCHLORIDE:Lung cancer metastatic:0.22), (CALCIUM CARBONATECHOLECALCIFEROL:Lung cancer metastatic:0.27), (CANDESARTAN CILEXETIL:Lung cancer metastatic:0.16), (DEXLANSOPRAZOLE:Lung cancer metastatic:0.53), (DICLOFENAC:Lung cancer metastatic:0.02), (EPINEPHRINE:Lung cancer metastatic:0.23), (EPIRUBICIN:Lung cancer metastatic:0.02), (IBRUTINIB:Lung cancer metastatic:0.83), (ILOPROST:Lung cancer metastatic:0.06), (LEVOLEUCOVORIN CALCIUM:Lung cancer metastatic:0.17), (MELOXICAM:Lung cancer metastatic:0.08), (METRONIDAZOLE:Lung cancer metastatic:0.15), (NEBIVOLOL:Lung cancer metastatic:0.34), (NILOTINIB:Lung cancer metastatic:0.05), (PANITUMUMAB:Lung cancer metastatic:0.19), (REGORAFENIB:Lung cancer metastatic:0.3), (TELMISARTAN:Lung cancer metastatic:0.17), (TINZAPARIN SODIUM:Lung cancer metastatic:0.12), (DILTIAZEM HYDROCHLORIDE:Lung carcinoma cell type unspecified stage 0:0.07), (RAMIPRIL:Lung carcinoma cell type unspecified stage 0:0.04), (ACEBUTOLOL HYDROCHLORIDE:Lung disorder:0.27), (ACETAMINOPHENCAFFEINE:Lung disorder:0.01), (ACETAMINOPHENDIPHENHYDRAMINE:Lung disorder:0.02), (ACETAMINOPHENOXYCODONEOXYCODONE HYDROCHLORIDE:Lung disorder:0.81), (AGALSIDASE BETA:Lung disorder:0.55), (ALBIGLUTIDE:Lung disorder:0.33), (ALBUTEROLGUAIFENESIN:Lung disorder:0.17), (ALCOHOL:Lung disorder:0.53), (ALEMTUZUMAB:Lung disorder:0.06), (ALENDRONIC ACID:Lung disorder:0.79), (ALISKIREN:Lung disorder:0.14), (ALISKIREN HEMIFUMARATE:Lung disorder:0.34), (ALUMINUM HYDROXIDEDIMETHICONEMAGNESIUM HYDROXIDE:Lung disorder:0.6), (AMANTADINE:Lung disorder:0.38), (AMANTADINE HYDROCHLORIDE:Lung disorder:0.16), (AMILORIDE HYDROCHLORIDEHYDROCHLOROTHIAZIDE:Lung disorder:0.38), (AMLODIPINE BESYLATEHYDROCHLOROTHIAZIDEVALSARTAN:Lung disorder:0.22), (ANTIHISTAMINES NOS:Lung disorder:0.02), (ASPIRINCLOPIDOGREL:Lung disorder:0.2), (AVANAFIL:Lung disorder:0.1), (AZILSARTAN KAMEDOXOMIL:Lung disorder:0.5), (BACITRACIN:Lung disorder:0.01), (BECLOMETHASONE:Lung disorder:0.25), (BECLOMETHASONE DIPROPIONATEFORMOTEROL:Lung disorder:0.08), (BELATACEPT:Lung disorder:0.22), (BENDROFLUMETHIAZIDE:Lung disorder:0.05), (BETAMETHASONECALCIPOTRIENE:Lung disorder:0.08), (BICALUTAMIDE:Lung disorder:0.69), (BLINATUMOMAB:Lung disorder:0.03), (BROTIZOLAM:Lung disorder:0.39), (CALCIPOTRIENE:Lung disorder:0.15), (CALCITONIN SALMON:Lung disorder:0.42), (CALCIUMMAGNESIUM:Lung disorder:0.25), (CANAGLIFLOZINMETFORMIN HYDROCHLORIDE:Lung disorder:0.61), (CANAKINUMAB:Lung disorder:0.69), (CASPOFUNGIN:Lung disorder:0.75), (CEFAZOLIN:Lung disorder:0.22), (CHLORAMBUCIL:Lung disorder:0.28), (CIMETIDINE:Lung disorder:0.95), (CINACALCET HYDROCHLORIDE:Lung disorder:0.59), (CIPROFIBRATE:Lung disorder:0.2), (CLINDAMYCIN:Lung disorder:1.69), (CLINDAMYCIN PHOSPHATE:Lung disorder:0.03), (CLOFAZIMINE:Lung disorder:0.03), (CYPROHEPTADINE:Lung disorder:0.57), (DABIGATRAN:Lung disorder:0.71), (DACLIZUMAB:Lung disorder:1.2), (DANAZOL:Lung disorder:0.62), (DARATUMUMAB:Lung disorder:0.34), (DASATINIB:Lung disorder:2.05), (DEOXYRIBONUCLIEC ACID:Lung disorder:0.06), (DEXAMETHASONE ACETATE:Lung disorder:0.74), (DIGITOXIN:Lung disorder:0.38), (DIPYRIDAMOLE:Lung disorder:0.6), (ELETRIPTAN HYDROBROMIDE:Lung disorder:1.3), (ELOTUZUMAB:Lung disorder:0.47), (EMPAGLIFLOZINLINAGLIPTIN:Lung disorder:0.75), (ESTROGENS, CONJUGATEDMEDROXYPROGESTERONE ACETATE:Lung disorder:0.51), (FENTANYL CITRATE:Lung disorder:0.07), (FERROUS SULFATEGLYCINE:Lung disorder:0.06), (FLUDROCORTISONE ACETATE:Lung disorder:0.56), (FLUOCINOLONE ACETONIDE:Lung disorder:0.17), (FORMOTEROL FUMARATEMOMETASONE FUROATE:Lung disorder:1.79), (FOSFOMYCIN:Lung disorder:0.55), (HERBALSSAW PALMETTO:Lung disorder:0.14), (HYDROCHLOROTHIAZIDELOSARTAN:Lung disorder:0.46), (HYDROXYCHLOROQUINE DIPHOSPHATE:Lung disorder:0.52), (IBRITUMOMAB TIUXETAN:Lung disorder:0.2), (INDAPAMIDEPERINDOPRIL:Lung disorder:2.12), (INFLIXIMAB-DYYB:Lung disorder:0.48), (INOTUZUMAB OZOGAMICIN:Lung disorder:0.06), (INSULIN BEEF:Lung disorder:0.15), (INTERFERON ALFA-2A:Lung disorder:0.21), (IRINOTECAN HYDROCHLORIDE:Lung disorder:0.48), (KETOROLACKETOROLAC TROMETHAMINE:Lung disorder:0.5), (LACTASE:Lung disorder:0.29), (LANREOTIDE ACETATE:Lung disorder:0), (LAPATINIB DITOSYLATE:Lung disorder:0.11), (LEVOLEUCOVORIN CALCIUM:Lung disorder:0.03), (LIDOCAINEPRILOCAINE:Lung disorder:0.83), (LISDEXAMFETAMINE DIMESYLATE:Lung disorder:0.49), (LUMACAFTOR:Lung disorder:0.61), (MELPERONE:Lung disorder:0.39), (MELPHALAN:Lung disorder:0.1), (METHENAMINE HIPPURATE:Lung disorder:0.85), (MIDODRINE HYDROCHLORIDE:Lung disorder:0.08), (MORPHINE SULFATENALTREXONE HYDROCHLORIDE:Lung disorder:0.32), (MOXONIDINE:Lung disorder:0.56), (NABUMETONE:Lung disorder:0.73), (NATEGLINIDE:Lung disorder:0.77), (NOREPINEPHRINE:Lung disorder:0.64), (NOREPINEPHRINE BITARTRATE:Lung disorder:0.11), (OLMESARTAN:Lung disorder:0.03), (OLOPATADINE:Lung disorder:0.49), (OXCARBAZEPINE:Lung disorder:2.65), (PANITUMUMAB:Lung disorder:0.21), (PATIROMER:Lung disorder:0.29), (PEGINTERFERON ALFA-2B:Lung disorder:0.15), (PEGINTERFERON BETA-1A:Lung disorder:0.58), (PEMETREXED:Lung disorder:0.27), (PENTAMIDINE ISETHIONATE:Lung disorder:0.72), (PENTOSAN POLYSULFATE SODIUM:Lung disorder:0.12), (PILOCARPINE:Lung disorder:0.09), (PIMECROLIMUS:Lung disorder:0.4), (PIROXICAM:Lung disorder:0.61), (PONATINIB:Lung disorder:1.24), (PRAZOSIN:Lung disorder:0.4), (PRAZOSIN HYDROCHLORIDE:Lung disorder:0.09), (PREDNISOLONE ACETATE:Lung disorder:0.78), (PROPAFENONE:Lung disorder:0.16), (RABEPRAZOLE:Lung disorder:1.28), (RALTEGRAVIR POTASSIUM:Lung disorder:0.27), (SUMATRIPTANSUMATRIPTAN SUCCINATE:Lung disorder:1.14), (TAZOBACTAM:Lung disorder:0.48), (TERBINAFINE HYDROCHLORIDE:Lung disorder:1), (TESTOSTERONE UNDECANOATE:Lung disorder:0.01), (TETRACYCLINE:Lung disorder:1.01), (TIMOLOL:Lung disorder:1.32), (TINZAPARIN:Lung disorder:0.6), (TRANDOLAPRIL:Lung disorder:0.09), (VILAZODONE HYDROCHLORIDE:Lung disorder:0.42), (VINCRISTINE SULFATE:Lung disorder:0.55), (VINORELBINE TARTRATE:Lung disorder:0.17), (VITAMIN A:Lung disorder:0.74), (.ALPHA.1-PROTEINASE INHIBITOR HUMAN:Lung infection:0.87), (ACARBOSE:Lung infection:0.05), (ACETAMINOPHENBUTALBITALCAFFEINE:Lung infection:0.24), (ACETAMINOPHENOXYCODONEOXYCODONE HYDROCHLORIDE:Lung infection:0.06), (ALCOHOL:Lung infection:0.11), (AMANTADINE:Lung infection:0), (AMISULPRIDE:Lung infection:0.25), (AMITRIPTYLINE HYDROCHLORIDE:Lung infection:0.25), (ANASTROZOLE:Lung infection:1.32), (BENAZEPRILBENAZEPRIL HYDROCHLORIDE:Lung infection:0.09), (BRINZOLAMIDE:Lung infection:0.01), (BUSPIRONE HYDROCHLORIDE:Lung infection:0.41), (CALCIUM ACETATE:Lung infection:0.39), (CANAKINUMAB:Lung infection:0.41), (CARVEDILOL PHOSPHATE:Lung infection:0.16), (CEFAZOLIN:Lung infection:0.3), (CEFOXITINCEFOXITIN SODIUM:Lung infection:0.23), (CHLORAMBUCIL:Lung infection:0.29), (CHLORTHALIDONE:Lung infection:0.1), (CHOLESTYRAMINE:Lung infection:0.95), (CYPROHEPTADINE:Lung infection:0.18), (DACLIZUMAB:Lung infection:0.42), (DANAZOL:Lung infection:0.34), (DEOXYRIBONUCLIEC ACID:Lung infection:0.02), (DEXAMETHASONE ACETATE:Lung infection:0.36), (DEXLANSOPRAZOLE:Lung infection:0.81), (DICYCLOMINE HYDROCHLORIDE:Lung infection:0.47), (DIGITOXIN:Lung infection:0.2), (DIPYRONE:Lung infection:0.03), (DOFETILIDE:Lung infection:0.02), (EDOXABAN TOSYLATE:Lung infection:0.14), (ELETRIPTAN HYDROBROMIDE:Lung infection:0.12), (ELOTUZUMAB:Lung infection:0.52), (ESZOPICLONE:Lung infection:0.98), (ETORICOXIB:Lung infection:0.19), (EVOLOCUMAB:Lung infection:0.21), (FENOTEROL HYDROBROMIDE:Lung infection:0.41), (FERROUS FUMARATE:Lung infection:0.06), (FESOTERODINE FUMARATE:Lung infection:0.32), (FORMOTEROL FUMARATEMOMETASONE FUROATE:Lung infection:1.11), (FOSFOMYCIN:Lung infection:0.22), (FUSIDIC ACID:Lung infection:0.12), (HYDROCHLOROTHIAZIDELISINOPRIL:Lung infection:0.7), (HYDROMORPHONE:Lung infection:1.01), (HYDROXYUREA:Lung infection:0.82), (HYDROXYZINE PAMOATE:Lung infection:0.63), (ILOPROST:Lung infection:0.38), (INFLUENZA VIRUS VACCINE:Lung infection:0.95), (INSULIN DETEMIR:Lung infection:0.25), (INTERFERON GAMMA-1B:Lung infection:0.05), (INVESTIGATIONAL PRODUCT:Lung infection:0.32), (IXAZOMIB:Lung infection:0.12), (LACOSAMIDE:Lung infection:0.01), (LATANOPROST:Lung infection:0.54), (LEDIPASVIRSOFOSBUVIR:Lung infection:0.24), (LEVALBUTEROL:Lung infection:0.34), (LIDOCAINEPRILOCAINE:Lung infection:0.23), (LUMACAFTOR:Lung infection:0.5), (MEGESTROL ACETATE:Lung infection:0.05), (MEPOLIZUMAB:Lung infection:0.87), (MESNA:Lung infection:0.03), (METHYLPREDNISOLONE ACETATE:Lung infection:0.34), (METOLAZONE:Lung infection:0.03), (NILOTINIB:Lung infection:1.62), (NORTRIPTYLINE:Lung infection:0.17), (OLOPATADINE:Lung infection:0.27), (OMEGA-3-ACID ETHYL ESTERS:Lung infection:0.3), (OXYBUTYNIN CHLORIDE:Lung infection:0.09), (PEMETREXED:Lung infection:0.12), (PHENOBARBITAL:Lung infection:0.81), (PHENYTOIN SODIUM:Lung infection:0.12), (PIPERACILLIN:Lung infection:0.03), (POLYETHYLENE GLYCOL 3350:Lung infection:0.28), (PONATINIB:Lung infection:0.5), (PRAMIPEXOLE:Lung infection:0.69), (PREDNISOLONE SODIUM PHOSPHATE:Lung infection:0.05), (PREDNISOLONEPREDNISOLONE ACETATEPREDNISOLONE SODIUM SUCCINATE:Lung infection:0.81), (QUETIAPINE FUMARATE:Lung infection:0.43), (RABEPRAZOLE:Lung infection:0.64), (RALTEGRAVIR POTASSIUM:Lung infection:0.14), (RANOLAZINE:Lung infection:0.63), (RILMENIDINE PHOSPHATE:Lung infection:0), (ROPINIROLE HYDROCHLORIDE:Lung infection:0.08), (SECUKINUMAB:Lung infection:0.64), (TELMISARTAN:Lung infection:1.25), (TERIFLUNOMIDE:Lung infection:0.16), (THEOPHYLLINE:Lung infection:0.06), (TIMOLOL:Lung infection:0.27), (TIZANIDINE HYDROCHLORIDE:Lung infection:0.36), (TOFACITINIB CITRATE:Lung infection:0.06), (VARENICLINE TARTRATE:Lung infection:0.12), (VITAMIN B:Lung infection:0.57), (ABIRATERONE:Lung neoplasm malignant:0.04), (ACITRETIN:Lung neoplasm malignant:0.63), (AGALSIDASE BETA:Lung neoplasm malignant:0.8), (ALCOHOL:Lung neoplasm malignant:0.5), (ALISKIREN:Lung neoplasm malignant:0.41), (AMANTADINE HYDROCHLORIDE:Lung neoplasm malignant:0.53), (AMINO ACIDSDEXTROSEELECTROLYTES NOS:Lung neoplasm malignant:0.43), (AMPICILLIN SODIUM:Lung neoplasm malignant:0.01), (ANAGRELIDE HYDROCHLORIDE:Lung neoplasm malignant:0.02),(ARMODAFINIL:Lung neoplasm malignant:1.44), (ASCORBIC ACIDBIOTINCYANOCOBALAMINFOLIC ACIDNIACINPANTOTHENIC ACIDPYRIDOXINERIBOFLAVINTHIAMINE:Lung neoplasm malignant:0.13), (ASPIRINDIHYDROXYALUMINUM AMINOACETATE ANHYDROUSMAGNESIUM CARBONATE:Lung neoplasm malignant:0.33), (ATOVAQUONE:Lung neoplasm malignant:0.41), (AZOSEMIDE:Lung neoplasm malignant:0.47), (BECLOMETHASONE:Lung neoplasm malignant:0.23), (BENDAMUSTINE:Lung neoplasm malignant:0.38), (BENDAMUSTINE HYDROCHLORIDE:Lung neoplasm malignant:0.06), (BENZOCAINE:Lung neoplasm malignant:0), (BETAMETHASONE DIPROPIONATECALCIPOTRIENECALCIPOTRIENE HYDRATE:Lung neoplasm malignant:0.51), (BETAMETHASONECALCIPOTRIENE:Lung neoplasm malignant:0.68), (BETAXOLOL HYDROCHLORIDE:Lung neoplasm malignant:0.17), (BETHANECHOL CHLORIDE:Lung neoplasm malignant:0.27), (BLINATUMOMAB:Lung neoplasm malignant:0.05), (BOCEPREVIR:Lung neoplasm malignant:0.01), (BOCOCIZUMAB:Lung neoplasm malignant:0.04), (BUDESONIDEFORMOTEROL:Lung neoplasm malignant:0.33), (BUPRENORPHINE HYDROCHLORIDE:Lung neoplasm malignant:0.66), (BUPRENORPHINE HYDROCHLORIDENALOXONE HYDROCHLORIDE:Lung neoplasm malignant:0.57), (BUTALBITAL:Lung neoplasm malignant:0.02), (CABAZITAXEL:Lung neoplasm malignant:0.34), (CALCIUMMAGNESIUMZINC:Lung neoplasm malignant:0.25), (CANAGLIFLOZINMETFORMIN HYDROCHLORIDE:Lung neoplasm malignant:1.24), (CANAKINUMAB:Lung neoplasm malignant:0.42), (CAPSAICIN:Lung neoplasm malignant:0.02), (CARBIMAZOLE:Lung neoplasm malignant:0.99), (CARBOMER:Lung neoplasm malignant:0.42), (CEFAZOLIN:Lung neoplasm malignant:0.54), (CEVIMELINE HYDROCHLORIDE:Lung neoplasm malignant:0.3), (CHLORAMBUCIL:Lung neoplasm malignant:0.46), (CILASTATIN SODIUMIMIPENEM:Lung neoplasm malignant:1.41), (CIPROFIBRATE:Lung neoplasm malignant:0.48), (CLOBAZAM:Lung neoplasm malignant:0.26), (CLOMIPRAMINE HYDROCHLORIDE:Lung neoplasm malignant:0.28), (COBICISTATELVITEGRAVIREMTRICITABINETENOFOVIR ALAFENAMIDE FUMARATE:Lung neoplasm malignant:0.09), (CORTICOTROPIN:Lung neoplasm malignant:0.76), (DACLIZUMAB:Lung neoplasm malignant:0.8), (DANAZOL:Lung neoplasm malignant:0.65), (DANTROLENE SODIUM:Lung neoplasm malignant:0.04), (DASABUVIROMBITASVIRPARITAPREVIRRITONAVIR:Lung neoplasm malignant:0.04), (DEGARELIX:Lung neoplasm malignant:0.22), (DESMOPRESSIN:Lung neoplasm malignant:0.09), (DEXTROAMPHETAMINE SULFATE:Lung neoplasm malignant:0.35), (DIGITOXIN:Lung neoplasm malignant:0.82), (DROXIDOPA:Lung neoplasm malignant:0.13), (EDOXABAN TOSYLATE:Lung neoplasm malignant:1.95), (ELETRIPTAN HYDROBROMIDE:Lung neoplasm malignant:1.73), (ELOTUZUMAB:Lung neoplasm malignant:1.02), (ELTROMBOPAG OLAMINE:Lung neoplasm malignant:0.12), (EMPAGLIFLOZINLINAGLIPTIN:Lung neoplasm malignant:0.65), (ENALAPRIL MALEATELERCANIDIPINE HYDROCHLORIDE:Lung neoplasm malignant:0.02), (EPIRUBICIN HYDROCHLORIDE:Lung neoplasm malignant:0.14), (ERIBULIN MESYLATE:Lung neoplasm malignant:0.42), (ESTRIOL:Lung neoplasm malignant:0.12), (ESTROGENS:Lung neoplasm malignant:0.57), (ESTRONESUS SCROFA OVARY:Lung neoplasm malignant:0.7), (ETORICOXIB:Lung neoplasm malignant:0.9), (ETRETINATE:Lung neoplasm malignant:0.58), (FERROUS SULFATE ANHYDROUS:Lung neoplasm malignant:0.14), (FERROUS SULFATEFOLIC ACID:Lung neoplasm malignant:0.62), (FLUOROURACILLEUCOVORIN CALCIUMOXALIPLATIN:Lung neoplasm malignant:0.14), (FLUTAMIDE:Lung neoplasm malignant:0.01), (FORMOTEROL FUMARATE DIHYDRATE:Lung neoplasm malignant:0.04), (FORMOTEROL FUMARATEMOMETASONE FUROATE:Lung neoplasm malignant:0.86), (GADOVERSETAMIDE:Lung neoplasm malignant:0.64), (GLIPIZIDEMETFORMIN HYDROCHLORIDE:Lung neoplasm malignant:0.09), (GLYCOPYRRONIUM:Lung neoplasm malignant:0.59), (GUANFACINE:Lung neoplasm malignant:0.22), (HYDROCHLOROTHIAZIDERAMIPRIL:Lung neoplasm malignant:0.08), (ILOPROST:Lung neoplasm malignant:0.88), (INFLIXIMAB-DYYB:Lung neoplasm malignant:0.65), (INOTUZUMAB OZOGAMICIN:Lung neoplasm malignant:0.11), (INSULIN BEEF:Lung neoplasm malignant:0.58), (INTERFERON:Lung neoplasm malignant:0.1), (IRON SUCROSE:Lung neoplasm malignant:0.73), (ITRACONAZOLE:Lung neoplasm malignant:0.93), (ITRACONAZOLESODIUM CHLORIDE:Lung neoplasm malignant:0.13), (IXAZOMIB:Lung neoplasm malignant:0.79), (LANREOTIDE:Lung neoplasm malignant:0.64), (LEDIPASVIRSOFOSBUVIR:Lung neoplasm malignant:1.56), (LEVOMEFOLATE CALCIUM:Lung neoplasm malignant:0.03), (LINSIDOMINE:Lung neoplasm malignant:0.11), (LISDEXAMFETAMINE DIMESYLATE:Lung neoplasm malignant:0.63), (LOMITAPIDE MESYLATE:Lung neoplasm malignant:0.76), (LUTETIUM LU-177:Lung neoplasm malignant:0.21), (MAGNESIUM ASPARTATEPOTASSIUM ASPARTATE:Lung neoplasm malignant:0.13), (MEPOLIZUMAB:Lung neoplasm malignant:0.6), (METFORMIN HYDROCHLORIDESAXAGLIPTIN:Lung neoplasm malignant:0.19), (MORPHINE SULFATENALTREXONE HYDROCHLORIDE:Lung neoplasm malignant:0.64), (NALOXONE:Lung neoplasm malignant:0), (NARATRIPTAN HYDROCHLORIDE:Lung neoplasm malignant:0.07), (NILOTINIB:Lung neoplasm malignant:2.93), (NOREPINEPHRINE:Lung neoplasm malignant:0.17), (NOREPINEPHRINE BITARTRATE:Lung neoplasm malignant:0.22), (NOREPINEPHRINENOREPINEPHRINE BITARTRATE:Lung neoplasm malignant:0), (ONABOTULINUMTOXINA:Lung neoplasm malignant:0.12), (ORPHENADRINE:Lung neoplasm malignant:0.46), (OXYBUTYNINOXYBUTYNIN CHLORIDE:Lung neoplasm malignant:0.3), (PANTOPRAZOLE MAGNESIUM:Lung neoplasm malignant:2.5), (PARATHYROID HORMONE:Lung neoplasm malignant:0.07), (PATIROMER:Lung neoplasm malignant:0.53), (PEGINTERFERON ALFA-2A:Lung neoplasm malignant:0.22), (PEGINTERFERON BETA-1A:Lung neoplasm malignant:0.48), (PENICILLIN V:Lung neoplasm malignant:0.4), (PENTOSTATIN:Lung neoplasm malignant:0.08), (PILOCARPINE:Lung neoplasm malignant:0.68), (PIMECROLIMUS:Lung neoplasm malignant:0.57), (POMALIDOMIDE:Lung neoplasm malignant:0.48), (POSACONAZOLE:Lung neoplasm malignant:0.24), (PRISTINAMYCIN:Lung neoplasm malignant:0.35), (PROTRIPTYLINE HYDROCHLORIDE:Lung neoplasm malignant:0), (PYRAZINAMIDE:Lung neoplasm malignant:0.75), (RADIUM RA-223 DICHLORIDE:Lung neoplasm malignant:0.11), (RASBURICASE:Lung neoplasm malignant:0.01), (RIFAXIMIN:Lung neoplasm malignant:0.68), (RIVASTIGMINE:Lung neoplasm malignant:0.84), (RIZATRIPTAN:Lung neoplasm malignant:0.14), (RIZATRIPTAN BENZOATE:Lung neoplasm malignant:1.41), (SACUBITRILVALSARTAN:Lung neoplasm malignant:0.48), (SECUKINUMAB:Lung neoplasm malignant:1.03), (SEVELAMER CARBONATE:Lung neoplasm malignant:0.47), (SIPULEUCEL-T:Lung neoplasm malignant:0.43), (SODIUM FERROUS CITRATE:Lung neoplasm malignant:0.01), (SODIUM OXYBATE:Lung neoplasm malignant:1.11), (SOFOSBUVIRVELPATASVIR:Lung neoplasm malignant:0.11), (SUMATRIPTAN SUCCINATE:Lung neoplasm malignant:0.23), (TAFLUPROST:Lung neoplasm malignant:0.33), (TEDUGLUTIDEWATER:Lung neoplasm malignant:0.17), (TELOTRISTAT ETHYL:Lung neoplasm malignant:0.49), (TERIFLUNOMIDE:Lung neoplasm malignant:0.49), (TESTOSTERONE UNDECANOATE:Lung neoplasm malignant:0.55), (TIMOLOL MALEATETRAVOPROST:Lung neoplasm malignant:0.16), (TOFACITINIB CITRATE:Lung neoplasm malignant:0.04), (TRABECTEDIN:Lung neoplasm malignant:0.27), (TRIPTORELIN PAMOATE:Lung neoplasm malignant:0.38), (VOGLIBOSE:Lung neoplasm malignant:0.24), (ZAFIRLUKAST:Lung neoplasm malignant:0.82), (ZIV-AFLIBERCEPT:Lung neoplasm malignant:0.37), (ZOLMITRIPTAN:Lung neoplasm malignant:0.43), (AMIODARONE HYDROCHLORIDE:Lung transplant:0.43), (CANDESARTAN:Lung transplant:0.28), (CELECOXIB:Lung transplant:0.06), (CEPHALEXIN:Lung transplant:0.36), (CLINDAMYCINCLINDAMYCIN PHOSPHATE:Lung transplant:0.46), (DACLIZUMAB:Lung transplant:0.13), (DIGOXIN:Lung transplant:0.72), (DOXYCYCLINE:Lung transplant:0.11), (IBRUTINIB:Lung transplant:0.34), (IBUPROFEN:Lung transplant:0.97), (ILOPROST:Lung transplant:0.28), (IMIPENEM:Lung transplant:0.1), (LIDOCAINE:Lung transplant:0.19), (LOVASTATIN:Lung transplant:0.38), (MELOXICAM:Lung transplant:0.23), (NAPROXEN:Lung transplant:0.26), (NEBIVOLOL:Lung transplant:0.44), (NITROFURANTOIN:Lung transplant:0.04), (NITROGLYCERIN:Lung transplant:0.12), (RANOLAZINE:Lung transplant:0.44), (TELMISARTAN:Lung transplant:0.01), (ACETAMINOPHENCODEINE PHOSPHATE:Lupus nephritis:0.66), (ACETAZOLAMIDE:Lupus nephritis:0.5), (ADENINE:Lupus nephritis:0.5), (AMINO ACIDSCARBOHYDRATESELECTROLYTES NOSVITAMINS:Lupus nephritis:0.41), (AMINO ACIDSCARBOHYDRATESMINERALSSOYBEAN OILVITAMINS:Lupus nephritis:0.19), (AMIODARONE:Lupus nephritis:0.21), (AMIODARONE HYDROCHLORIDE:Lupus nephritis:0.18), (AMOXAPINE:Lupus nephritis:0.92), (ANAGLIPTIN:Lupus nephritis:0.54), (ASPIRINLANSOPRAZOLE:Lupus nephritis:0.16), (AZELNIDIPINEOLMESARTAN MEDOXOMIL:Lupus nephritis:0.46), (BACITRACINNEOMYCIN SULFATE:Lupus nephritis:0.06), (BEPRIDIL:Lupus nephritis:1.55), (BERAPROST:Lupus nephritis:0.39), (BETAMETHASONE BUTYRATE PROPIONATE:Lupus nephritis:0.73), (BETAMETHASONE SODIUM PHOSPHATENEOMYCIN SULFATE:Lupus nephritis:0.67), (BETAMETHASONEDEXCHLORPHENIRAMINE MALEATE:Lupus nephritis:0.05), (BIFIDOBACTERIUM LONGUM:Lupus nephritis:0.11), (BISACODYL OR DOCUSATE SODIUM:Lupus nephritis:0.22), (BUPROPION HYDROCHLORIDE:Lupus nephritis:0.74), (CALCIUM CHLORIDE ANHYDROUSPOTASSIUM CHLORIDESODIUM CHLORIDESODIUM LACTATE:Lupus nephritis:0.19), (CAMOSTAT MESYLATE:Lupus nephritis:0.07), (CANAKINUMAB:Lupus nephritis:0.48), (CEFOTIAM HYDROCHLORIDE:Lupus nephritis:0.28), (CERTOLIZUMAB PEGOL:Lupus nephritis:1.38), (CHOLESTYRAMINE:Lupus nephritis:0.12), (CLENBUTEROL HYDROCHLORIDE:Lupus nephritis:0.44), (CLOTRIMAZOLE:Lupus nephritis:0.16), (CLOZAPINE:Lupus nephritis:0.52), (DALTEPARIN SODIUM:Lupus nephritis:0.16), (DEFLAZACORT:Lupus nephritis:0.41), (DESLORATADINE:Lupus nephritis:0.25), (DEXAMETHASONE ACETATE:Lupus nephritis:0.89), (DOBUTAMINE HYDROCHLORIDE:Lupus nephritis:0.02), (ECABET:Lupus nephritis:0.21), (ECABET SODIUM:Lupus nephritis:0.54), (ELBASVIR:Lupus nephritis:0.09), (ENOXAPARIN:Lupus nephritis:1.38), (EPALRESTAT:Lupus nephritis:0.04), (ERYTHROMYCIN:Lupus nephritis:0.21), (FERRIC OXYHYDROXIDE:Lupus nephritis:0.23), (FERROUS SULFATEGLYCINE:Lupus nephritis:0.23), (FLUTICASONE:Lupus nephritis:0.01), (FLUTICASONE PROPIONATEFORMOTEROL FUMARATE:Lupus nephritis:0.12), (FURSULTIAMINE:Lupus nephritis:0.56), (GENTAMICIN:Lupus nephritis:0.22), (GLUTAMINESODIUM GUALENATE:Lupus nephritis:0.2), (GRAZOPREVIR:Lupus nephritis:0.07), (HYDROCHLOROTHIAZIDEIRBESARTAN:Lupus nephritis:0.14), (HYDROXYZINE PAMOATE:Lupus nephritis:0.15), (IBRUTINIB:Lupus nephritis:0.37), (IGURATIMOD:Lupus nephritis:1.07), (IMIDAFENACIN:Lupus nephritis:0.15), (IPRATROPIUM:Lupus nephritis:0.15), (IRON:Lupus nephritis:0.43), (ITOPRIDE:Lupus nephritis:0.08), (KALLIDINOGENASE:Lupus nephritis:0.35), (LERCANIDIPINE:Lupus nephritis:0.2), (LIDOCAINE:Lupus nephritis:0.38), (LOVASTATIN:Lupus nephritis:0.29), (LOXOPROFEN SODIUM DIHYDRATE:Lupus nephritis:0.49), (LYSOZYME HYDROCHLORIDE:Lupus nephritis:1.56), (MAGNESIUM:Lupus nephritis:0.55), (METHADONE HYDROCHLORIDE:Lupus nephritis:0.08), (METHENOLONE ENANTHATE:Lupus nephritis:0.61), (METHIMAZOLE:Lupus nephritis:0. 11), (METILDIGOXIN:Lupus nephritis:0.48), (METRONIDAZOLE:Lupus nephritis:0.88), (MITIGLINIDE CALCIUM HYDRATEVOGLIBOSE:Lupus nephritis:0.2), (MOSAPRIDE CITRATE DIHYDRATE:Lupus nephritis:0.67), (NAFTOPIDIL:Lupus nephritis:0.47), (NEBIVOLOL:Lupus nephritis:0.35), (NILOTINIB:Lupus nephritis:0.61), (OXCARBAZEPINE:Lupus nephritis:1.34), (PENICILLIN:Lupus nephritis:0.08), (PILSICAINIDE HYDROCHLORIDE:Lupus nephritis:1.39), (PIMOBENDAN:Lupus nephritis:1.14), (PIRENOXINE:Lupus nephritis:0.04), (POLYETHYLENE GLYCOLS:Lupus nephritis:0.25), (POLYSTYRENE:Lupus nephritis:0.29), (POLYSTYRENE SULFONIC ACID:Lupus nephritis:0.08), (POTASSIUM:Lupus nephritis:0.61), (POTASSIUM IODIDE:Lupus nephritis:0.03), (PRIMIDONE:Lupus nephritis:0.63), (PROBUCOL:Lupus nephritis:0.11), (PROMETHAZINEPROMETHAZINE HYDROCHLORIDE:Lupus nephritis:0.05), (PROPIVERINE:Lupus nephritis:0.33), (RABEPRAZOLE:Lupus nephritis:0.16), (RANOLAZINE:Lupus nephritis:0.2), (ROFLUMILAST:Lupus nephritis:0.07), (SECUKINUMAB:Lupus nephritis:1.22), (SODIUM GUALENATE MONOHYDRATE:Lupus nephritis:0.44), (SOTALOL:Lupus nephritis:0.13), (STREPTOMYCIN SULFATE:Lupus nephritis:0.52), (SUPLATAST TOSILATE:Lupus nephritis:1.07), (TIZANIDINE HYDROCHLORIDE:Lupus nephritis:0.2), (TOFACITINIB CITRATE:Lupus nephritis:0.05), (TOPIRAMATE:Lupus nephritis:1.17), (VONOPRAZAN:Lupus nephritis:0.24), (NAPROXEN:Lupus-like syndrome:0.09), (BUDESONIDE:Lyme disease:0.01), (DIVALPROEX SODIUM:Lyme disease:0.04), (FOLIC ACID:Lyme disease:0.89), (PYRAZINAMIDE:Lymphadenitis bacterial:0.52), (CASPOFUNGIN:Lymphocytic leukaemia:0.05), (IBRITUMOMAB TIUXETAN:Lymphocytic leukaemia:0.51), (IDARUBICIN:Lymphocytic leukaemia:0.95), (IDARUBICIN HYDROCHLORIDE:Lymphocytic leukaemia:0.36), (RAMIPRIL:Lymphocytic leukaemia:0.29), (SACUBITRILVALSARTAN:Lymphocytic leukaemia:0.17), (TORSEMIDE:Lymphocytic leukaemia:0.22), (VINDESINE SULFATE:Lymphocytic leukaemia:0.11), (VINCRISTINE SULFATE:Lymphocytic lymphoma:0.27), (DIPHENHYDRAMINE:Lymphoedema:0.03), (EVOLOCUMAB:Lymphoede ma:0.33), (INSULIN LISPRO:Lymphoedema:0.13), (LANSOPRAZOLE:Lymphoedema:0.25), (MELOXICAM:Lymp hoedema:0.11), (NIFEDIPINE:Lymphoedema:0.09), (PRAVASTATIN SODIUM:Lymphoedema:0.02), (RANOLAZINE:Lymphoedema:0.1), (TAMSULOSIN:Lymphoe dema:0.23), (.ALPHA.1-PROTEINASE INHIBITOR HUMAN:Lymphoma:0.28), (ACENOCOUMAROL:Lymphoma:0.07), (ACLIDINIUM BROMIDE:Lymphoma:0.12), (ADO-TRASTUZUMAB EMTANSINE:Lymphoma:1.1), (ALCOHOL:Lymphoma:0.27), (ALISKIREN HEMIFUMARATE:Lymphoma:0), (AMANTADINE:Lymphoma:0.44), (AMILORIDE HYDROCHLORIDEHYDROCHLOROTHIAZIDE:Lymphoma:0.21), (AMISULPRIDE:Lymph oma:0.52), (AMLODIPINEPERINDOPRIL:Lymphoma:0.01), (AMLODIPINEVALSARTAN:L ymphoma:0.33), (AMMONIUM LACTATE:Lymphoma:0.04), (AMPICILLIN SODIUMSULBACTAM SODIUM:Lymphoma:0), (ATAZANAVIR:Lymphoma:0), (AZILSARTAN KAMEDOXOMIL:Lymphoma:0.26), (BENAZEPRILBENAZEPRIL HYDROCHLORIDE:Lymphoma:0.71), (BETAMETHASONE DIPROPIONATE:Lymphoma:0.46), (BICALUTAMIDE:Lymphoma:0.08), (BUPRENORPHINE HYDROCHLORIDE:Lymphoma:0.43), (BUPRENORPHINE HYDROCHLORIDENALOXONE HYDROCHLORIDE:Lymphoma:0.25), (CABAZITAXEL:Lymphoma:0.02), (CALCIPOTRIEN E:Lymphoma:0.04), (CALCITONIN:Lymphoma:0.37), (CALCITONIN SALMON:Lymphoma:0.73), (CALCIUM CARBONATEVITAMIN D:Lymphoma:0.31), (CANAGLIFLOZIN:Lymphoma:0.03), (CANAGLIFLOZINMETFORMIN HYDROCHLORIDE:Lymphoma:0), (CANAKINUMAB:Lymphoma:0.64), (CAPSAICIN:Lymp homa:0.21), (CEFAZOLIN:Lymphoma:0.12), (CERTOLIZUMAB PEGOL:Lymphoma:1.8), (CETUXIMAB:Lymphoma:0.99), (CILOSTAZOL:Lymphoma:0.28), (CLINDAMYCIN HYDROCHLORIDE:Lymphoma:0.02), (CLOBAZAM:Lymphoma:0.29), (CLOFARABINE:Ly mphoma:0.38), (CLOSTRIDIUM BUTYRICUM SPORES STRAIN M-55:Lymphoma:0.18), (CORTICOTROPIN:Lymphoma:0.01), (CYPROHEPTADINE:Lymphoma: 0.43), (DABIGATRAN:Lymphoma:0.31), (DABRAFENIB:Lymphoma:0.85), (DARATUMUMA B:Lymphoma:0.58), (DECITABINE:Lymphoma:0.39), (DEXAMETHASONE ACETATE:Lymphoma:1.01), (DICLOFENAC:Lymphoma:1.64), (DIGITOXIN:Lymphoma:0.2), (DIPYRIDAMOLE:Lymphoma:0.29), (ECONAZOLE:Lymphoma:0.75), (ERIBULIN:Lymphom a:0.12), (ESTROGENS, CONJUGATEDMEDROXYPROGESTERONE ACETATE:Lymphoma:0.13), (ETORICOXIB:Lymphoma:0.29), (EVOLOCUMAB:Lymphoma: 0.8), (FLUDROCORTISONE ACETATE:Lymphoma:0.23), (FLUTICASONE FUROATESALMETEROL XINAFOATE:Lymphoma:0.86), (FORMOTEROL FUMARATE:Lymphoma:0.55), (FUSIDIC ACID:Lymphoma:0.8), (GEFITINIB:Lymphoma:0.14), (GIMERACILOTERACILTEGAFUR: Lymphoma:0.71), (GLYCOPYRROLATE:Lymphoma:0.01), (GLYCOPYRRONIUMINDACAT EROL:Lymphoma:0.57), (HEPARIN CALCIUM:Lymphoma:0.07), (HYDROCHLOROTHIAZIDETELMISARTAN:Lymphoma:0.42), (HYDROXYCHLOROQUINE DIPHOSPHATE:Lymphoma:0.1), (HYOSCYAMINE:Lymphoma:0.29), (ILOPROST:Lymphom a:0.31), (INDAPAMIDEPERINDOPRIL:Lymphoma:0.98), (INOTUZUMAB OZOGAMICIN:Lymphoma:0.73), (INTERFERON ALFA-2A:Lymphoma:0.43), (INTERFERON GAMMA-1B:Lymphoma:0.41), (IPRATROPIUM:Lymphoma:1.41), (LEVOLEUCOVORIN CALCIUM:Lymphoma:0.49), (LIOTHYRONINE SODIUM:Lymphoma:0.03), (METAMIZOLE:Lymphoma:0.22), (METAXALONE:Lymphoma:10.17), (METFORMIN HYDROCHLORIDEVILDAGLIPTIN:Lymphoma:0.4), (METHENAMINE HIPPURATE:Lymphoma:0.51), (METHENOLONE ENANTHATE:Lymphoma:0.32), (METHIMAZOLE:Lymphoma:0.65), (MORPHINE SULFATENALTREXONE HYDROCHLORIDE:Lymphoma:0.05), (NABUMETONE:Lymphoma:0.34), (NILOTINIB:Lym phoma:2.52), (NINTEDANIB:Lymphoma:0.45), (NIRAPARIB:Lymphoma:0.01), (NORTRIPTY LINE HYDROCHLORIDE:Lymphoma:0.05), (OLODATEROL HYDROCHLORIDETIOTROPIUM BROMIDE MONOHYDRATE:Lymphoma:0.36), (PEGINTERFERON ALFA-2B:Lymphoma:0.11), (PEGINTERFERON BETA-1A:Lymphoma:0.17), (PENICILLIN G:Lymphoma:0.19), (PENTOSTATIN:Lymphoma:0.72), (PERTUZUMAB:Lymphoma:0.44), (P HENYTOIN SODIUM:Lymphoma:0.52), (PIROXICAM:Lymphoma:0.41), (PREDNISOLONE ACETATE:Lymphoma:0.49), (RAMUCIRUMAB:Lymphoma:0.35), (REGORAFENIB:Lympho ma:0.69), (RIVASTIGMINE:Lymphoma:0.22), (ROFLUMILAST:Lymphoma:0.79), (SALMETE ROL:Lymphoma:0.09), (SECUKINUMAB:Lymphoma:1.04), (SODIUM OXYBATE:Lymphoma:0.68), (SUMATRIPTANSUMATRIPTAN SUCCINATE:Lymphoma:0.82), (TERIFLUNOMIDE:Lymphoma:0), (TETRACYCLINE:Lymph oma:1.29), (THIOGUANINE:Lymphoma:0.04), (TINZAPARIN:Lymphoma:0.38), (TIZANIDIN E HYDROCHLORIDE:Lymphoma:1.18), (TOCOPHEROL:Lymphoma:0.1), (TRAMETINIB:Lym phoma:0.75), (TRETINOIN:Lymphoma:0.03), (TRIAMTERENE:Lymphoma:0.35), (UMECLIDI NIUM BROMIDE:Lymphoma:0.02), (UMECLIDINIUM BROMIDEVILANTEROL TRIFENATATE:Lymphoma:0.61), (VARENICLINE TARTRATE:Lymphoma:1.21), (EMTRICITABINE:Lymphoma AIDS related:0.65), (ACARBOSE:Macular degeneration:0.3), (ACETAMINOPHENHYDROCODONE:Macular degeneration:0.37), (ACETAMINOPHENOXYCODONE HYDROCHLORIDEOXYCODONE TEREPHTHALATE:Macular degeneration:0.36), (ACETAMINOPHENOXYCODONEOXYCODONE HYDROCHLORIDE:Macular degeneration:0.39), (ALBIGLUTIDE:Macular degeneration:0.1), (ALISKIREN HEMIFUMARATE:Macular degeneration:0.24), (AMANTADINE:Macular degeneration:0.2), (AMLODIPINEVALSARTAN:Macular degeneration:0.02), (AMMONIUM LACTATE:Macular degeneration:0), (AMPHETAMINE ASPARTATEAMPHETAMINE SULFATEDEXTROAMPHETAMINE SACCHARATEDEXTROAMPHETAMINE SULFATE:Macular degeneration:1.09), (BECLOMETHASONE DIPROPIONATE:Macular degeneration:0.13), (BICALUTAMIDE:Macular degeneration:0.04), (BROMAZEPAM:Macular degeneration:0.21), (CALCIUM ACETATE:Macular degeneration:0.77), (CALCIUMMAGNESIUMZINC:Macular degeneration:0.07), (CANAGLIFLOZINMETFORMIN HYDROCHLORIDE:Macular degeneration:0.13), (CARVEDILOL PHOSPHATE:Macular degeneration:1.06), (CERTOLIZUMAB PEGOL:Macular degeneration:1.4), (CICLESONIDE:Macular degeneration:0.21), (CIMETIDINE:Macular degeneration:0.58), (CINACALCET HYDROCHLORIDE:Macular degeneration:0.56), (CLOTRIMAZOLE:Macular degeneration:0.89), (CODEINE:Macular degeneration:0.07), (CORTICOTROPIN:Macular degeneration:0.03), (DABIGATRAN:Macular degeneration:0.02), (DACLIZUMAB:Macular degeneration:0.15), (DAPAGLIFLOZIN PROPANEDIOL:Macular degeneration:0.61), (DASATINIB:Macular degeneration:1.4), (DEFLAZACORT:Macular degeneration:0.02), (DESLORATADINE:Macular degeneration:0.55), (DEVICE:Macular degeneration:0.74), (DIPHENHYDRAMINE:Macular degeneration:0.94), (DRONABINOL:Macular degeneration:0.5), (DUTASTERIDETAMSULOSIN HYDROCHLORIDE:Macular degeneration:0), (EDOXABAN TOSYLATE:Macular degeneration:0.57), (ELETRIPTAN HYDROBROMIDE:Macular degeneration:0.93), (EMPAGLIFLOZINLINAGLIPTIN:Macular degeneration:0.3), (ENALAPRIL MALEATE:Macular degeneration:1.12), (ESOMEPRAZOLE MAGNESIUMNAPROXEN:Macular degeneration:0.1), (FLUOCINOLONE ACETONIDE:Macular degeneration:0.49), (FLUTICASONE FUROATE:Macular degeneration:0.26), (FLUTICASONE FUROATESALMETEROL XINAFOATE:Macular degeneration:0.16), (FULVESTRANT:Macular degeneration:0.34), (GLYCOPYRRONIUMINDACATEROL:Macular degeneration:0.45), (HERBALSTURMERIC:Macular degeneration:0.7), (HYDROCHLOROTHIAZIDETELMISARTAN:Macular degeneration:0.3), (HYDROXYZINE PAMOATE:Macular degeneration:1.09), (ILOPROST:Macular degeneration:0.26), (IMATINIB MESYLATE:Macular degeneration:0.1), (INDAPAMIDEPERINDOPRIL:Macular degeneration:0.59), (INFLUENZA VIRUS VACCINE:Macular degeneration:0.2), (INSULIN GLULISINE:Macular degeneration:0.06), (LERCANIDIPINE HYDROCHLORIDE:Macular degeneration:0.45), (LISDEXAMFETAMINE DIMESYLATE:Macular degeneration:0.25), (LITHIUM CARBONATE:Macular degeneration:0.53), (LOMITAPIDE MESYLATE:Macular degeneration:0.41), (MEGESTROL ACETATE:Macular degeneration:0.26), (METAXALONE:Macular degeneration:1.27), (METFORMIN HYDROCHLORIDEVILDAGLIPTIN:Macular degeneration:0), (METHYLPREDNISOLONE ACETATE:Macular degeneration:0.76), (METHYLPREDNISOLONE SODIUM SUCCINATE:Macular degeneration:0.83), (METRONIDAZOLEMETRONIDAZOLE HYDROCHLORIDE:Macular degeneration:0.31), (MIDODRINE:Macular degeneration:0.22), (MODAFINIL:Macular degeneration:0.87), (NALOXONE HYDROCHLORIDEOXYCODONE HYDROCHLORIDE:Macular degeneration:0.68), (NATEGLINIDE:Macular degeneration:0.28), (NILOTINIB:Macular degeneration:0.86), (OLODATEROL HYDROCHLORIDETIOTROPIUM BROMIDE MONOHYDRATE:Macular degeneration:0.42), (PAROXETINE:Macular degeneration:0.25), (PAZOPANIB HYDROCHLORIDE:Macular degeneration:0.79), (PEGINTERFERON BETA-1A:Macular degeneration:0.26), (PEMETREXED DISODIUM:Macular degeneration:0.15), (PENICILLIN:Macular degeneration:0.44), (PIROXICAM:Macular degeneration:0.16), (PONATINIB:Macular degeneration:0.11), (PYRIDOXINE:Macular degeneration:0.05), (RIZATRIPTAN BENZOATE:Macular degeneration:0.28), (SCOPOLAMINE:Macular degeneration:0.31), (SECUKINUMAB:Macular degeneration:0.43), (SUMATRIPTAN:Macular degeneration:0.27), (SUMATRIPTANSUMATRIPTAN SUCCINATE:Macular degeneration:0.53), (TERBINAFINE HYDROCHLORIDE:Macular degeneration:0.37), (TERBUTALINE SULFATE:Macular degeneration:0.29), (TERIFLUNOMIDE:Macular degeneration:0.46), (TETRACYCLINE:Macular degeneration:0.42), (THALIDOMIDE:Macular degeneration:0.61), (TIMOLOL MALEATETRAVOPROST:Macular degeneration:0.07), (UMECLIDINIUM BROMIDE:Macular degeneration:0.01), (ZONISAMIDE:Macular degeneration:0.01), (AMIODARONE:Macular oedema:0.06), (AMIODARONE HYDROCHLORIDE:Macular oedema:0.09), (AZITHROMYCIN MONOHYDRATE:Macular oedema:0.1), (CARTEOLOL HYDROCHLORIDE:Macular oedema:0.31), (CELECOXIB:Macular oedema:0.01), (DEXLANSOPRAZOLE:Macular oedema:0.13), (EVEROLIMUS:Macular oedema:0.06), (EVOLOCUMAB:Macular oedema:0.28), (METOPROLOL TARTRATE:Macular oedema:0.05), (OXYGEN:Macular oedema:0.03), (PANTOPRAZOLE:Macular oedema:0.23), (TIMOLOL MALEATE:Macular oedema:0.02), (TIMOLOL MALEATETRAVOPROST:Macular oedema:0.09), (TIMOLOLTRAVOPROST:Macular oedema:0.07), (TRAMADOL:Macular oedema:0.18), (VERAPAMIL HYDROCHLORIDE:Macular oedema:0.09), (BUPROPION HYDROCHLORIDE:Magnesium deficiency:0.36), (CANDESARTAN CILEXETIL:Magnesium deficiency:0.09), (CANDESARTAN CILEXETILHYDROCHLOROTHIAZIDE:Magnesium deficiency:0.05), (DEVICE:Magnesium deficiency:0.01), (DEXLANSOPRAZOLE:Magnesium deficiency:0.42), (DICLOFENAC SODIUM:Magnesium deficiency:0.13), (DULOXETINE HYDROCHLORIDE:Magnesium deficiency:0.05), (ENALAPRIL:Magnesium deficiency:0.78), (EVOLOCUMAB:Magnesium deficiency:0.34), (EZETIMIBE:Magnesium deficiency:0.01), (GLIPIZIDE:Magnesium deficiency:0.2), (IBRUTINIB:Magnesium deficiency:0.58), (IBUPROFEN:Magnesium deficiency:0.55), (LEDIPASVIRSOFOSBUVIR:Magnesium deficiency:0.18), (LERCANIDIPINE:Magnesium deficiency:0.06), (METHYLPREDNISOLONE SODIUM SUCCINATE:Magnesium deficiency:0.07), (OXYGEN:Magnesium deficiency:0.39), (RISEDRONATE SODIUM:Magnesium deficiency:0.2), (SACUBITRILVALSARTAN:Magnesium deficiency:0.35), (SOTALOL:Magnesium deficiency:0.06), (CARBON DIOXIDE:Maintenance of anaesthesia:0.12), (DIVALPROEX SODIUM:Maintenance of anaesthesia:0.49), (FOSPHENYTOINFOSPHENYTOIN SODIUM:Maintenance of anaesthesia:0.77), (METARAMINOL:Maintenance of anaesthesia:0.21), (OXCARBAZEPINE:Maintenance of anaesthesia:0.81), (TOPIRAMATE:Maintenance of anaesthesia:0.28), (ABACAVIR SULFATEDOLUTEGRAVIR SODIUMLAMIVUDINE:Major depression:0.42), (ABIRATERONE ACETATE:Major depression:0.06), (ACETAMINOPHENOXYCODONEOXYCODONE HYDROCHLORIDE:Major depression:1.05), (ACLIDINIUM BROMIDE:Major depression:0.57), (ALENDRONIC ACID:Major depression:0.38), (ALFUZOSIN HYDROCHLORIDE:Major depression:0.45), (ALUMINUM HYDROXIDEDIMETHICONEMAGNESIUM HYDROXIDE:Major depression:0.19), (AMILORIDE HYDROCHLORIDEHYDROCHLOROTHIAZIDE:Major depression:0.14), (AMIODARONE:Major depression:1.46), (AMLODIPINE BESYLATEHYDROCHLOROTHIAZIDEOLMESARTAN MEDOXOMIL:Major depression:0.29), (AMLODIPINEVALSARTAN:Major depression:0.15), (AMMONIUM LACTATE:Major depression:0.09), (ANTIHISTAMINES NOS:Major depression:0.01), (ATOMOXETINE:Major depression:1.06), (BIFIDOBACTERIUM LONGUM SUBSP. INFANTIS:Major depression:0.57), (BRINZOLAMIDE:Major depression:0.27), (BUDESONIDEFORMOTEROL FUMARATE:Major depression:0.32), (BUTYLSCOPOLAMINE BROMIDE:Major depression:0.97), (CALCIUM ACETATE:Major depression:0.65), (CALCIUM CITRATEVITAMIN D:Major depression:0.21), (CALCIUMMAGNESIUMZINC:Major depression:0.27), (CANAGLIFLOZINMETFORMIN HYDROCHLORIDE:Major depression:0.5), (CANDESARTAN CILEXETIL:Major depression:1.59), (CANDESARTAN CILEXETILHYDROCHLOROTHIAZIDE:Major depression:1.3), (CANNABIS SATIVA SUBSP. SATIVA FLOWERING TOP:Major depression:0.11), (CAPSAICIN:Major depression:0.06), (CARFILZOMIB:Major depression:0.24), (CARVEDILOL PHOSPHATE:Major depression:1.06), (CERTOLIZUMAB PEGOL:Major depression:1.42), (CICLESONIDE:Major depression:0.36), (CLOMETHIAZOLE:Major depression:0.16), (COD LIVER OIL:Major depression:0.22), (CORTICOTROPIN:Major depression:0.51), (CYPROHEPTADINE:Major depression:0.94), (DABIGATRAN:Major depression:0.31), (DAPAGLIFLOZIN PROPANEDIOL:Major depression:0.78), (DASATINIB:Major depression:0.86), (DIPYRONE:Major depression:0.21), (DUTASTERIDETAMSULOSIN HYDROCHLORIDE:Major depression:0.15), (EDOXABAN TOSYLATE:Major depression:0.34), (EPLERENONE:Major depression:0.57), (ERYTHROMYCIN:Major depression:0.13), (ESTRADIOLNORETHINDRONE ACETATE:Major depression:0.17), (ETORICOXIB:Major depression:0.68), (EVOLOCUMAB:Major depression:1.82), (FEXOFENADINEPSEUDOEPHEDRINE:Major depression:0.41), (FLUDROCORTISONE:Major depression:0.24), (FLUPENTIXOL DECANOATE:Major depression:0.21), (FLUPHENAZINE DECANOATE:Major depression:0.35), (FLUPHENAZINE HYDROCHLORIDE:Major depression:0.23), (FLUTICASONE FUROATESALMETEROL XINAFOATE:Major depression:0.64), (FORMOTEROL FUMARATE:Major depression:0.42), (FUSIDIC ACID:Major depression:0.55), (HYDROCODONE BITARTRATEIBUPROFEN:Major depression:0.31), (ILOPROST:Major depression:0.03), (IMATINIB MESYLATE:Major depression:0.62), (INSULIN DEGLUDEC:Major depression:0.34), (LACTASE:Major depression:0.16), (LERCANIDIPINE:Major depression:1.16), (LITHIUM SULFATE:Major depression:0.66), (MAGNESIUM HYDROXIDE:Major depression:0.05), (MEGESTROL ACETATE:Major depression:0.32), (MEPERIDINE:Major depression:0.03), (METHENAMINE HIPPURATE:Major depression:0.91), (METHYLENEDIOXYMETHAMPHETAMINE:Major depression:0.02), (MIDODRINE:Major depression:0.41), (MORPHINEMORPHINE HYDROCHLORIDEMORPHINE SULFATEMORPHINE TARTRATE:Major depression:0.11), (NALOXONE:Major depression:0.01), (NALOXONE HYDROCHLORIDEOXYCODONE HYDROCHLORIDE:Major depression:1.1), (NATEGLINIDE:Major depression:0.06), (NILOTINIB:Major depression:0.59), (OSPEMIFENE:Major depression:0.76), (OXAPROZIN:Major depression:0.02), (PANTOPRAZOLE MAGNESIUM:Major depression:1.1), (PAZOPANIB HYDROCHLORIDE:Major depression:0.65), (PEGINTERFERON BETA-1A:Major depression:0.59), (PERAMPANEL:Major depression:0.32), (PHENYTOIN SODIUM:Major depression:0.54), (PIRIBEDIL:Major depression:0.21), (PONATINIB:Major depression:0.13), (PROPAFENONE HYDROCHLORIDE:Major depression:0.21), (PSYLLIUM HUSK:Major depression:0.36), (ROFECOXIB:Major depression:0.41), (SACCHAROMYCES CEREVISIAE:Major depression:0.13), (SERTINDOLE:Major depression:0.42), (SILODOSIN:Major depression:0.33), (TERBUTALINE:Major depression:0.06), (TERBUTALINE SULFATE:Major depression:0.82), (TERIFLUNOMIDE:Major depression:1.15), (TIOTROPIUM:Major depression:0.68), (TOLTERODINE:Major depression:0.04), (TRIFLUOPERAZINE HYDROCHLORIDE:Major depression:0.02), (TROSPIUM CHLORIDE:Major depression:0.36), (VALGANCICLOVIR HYDROCHLORIDE:Major depression:0.37), (ZAFIRLUKAST:Major depression:0.02), (CERTOLIZUMAB PEGOL:Malabsorption:0.28), (CIPROFLOXACIN HYDROCHLORIDE:Malabsorption:0.13), (EVEROLIMUS:Malabsorption:0.04), (LOVASTATI N:Malabsorption:0.16), (TERIFLUNOMIDE:Malabsorption:0.28), (.ALPHA.1-PROTEINASE INHIBITOR HUMAN:Malaise:0.37), (ACETAMINOPHENBUTALBITALCAFFEINE:Malaise:0.74), (ACE TAMINOPHENHYDROCODONE:Malaise:0.14), (ALBUTEROL SULFATEIPRATROPIUM BROMIDE:Malaise:0.92), (AMIODARONE HYDROCHLORIDE:Malaise:0.52), (AMISULPRIDE:Malaise:0.27), (BENAZEPRIL HYDROCHLORIDE:Malaise:0.51), (BICALUTAMIDE:Malaise:0.41), (BUPRENORPHINE HYDROCHLORIDE:Malaise:0.52), (BUPRENORPHINE HYDROCHLORIDENALOXONE HYDROCHLORIDE:Malaise:0.39), (CANAKINUMAB:Malaise:0.17), (CANDESARTAN:Mala ise:1. 18), (CANDESARTAN CILEXETILHYDROCHLOROTHIAZIDE:Malaise:0.65), (CAPTOPRIL:Malaise:0.02), (CILAS TATIN SODIUMIMIPENEM:Malaise:0.29), (DABRAFENIB:Malaise:0.12), (DACLIZUMAB:Malaise: 0.2), (DESLORATADINE:Malaise:0.62), (DEVICE:Malaise:0.06), (DEXAMETHASONE ACETATE:Malaise:0.22), (DICYCLOMINE HYDROCHLORIDE:Malaise:0.42), (DIVALPROEX SODIUM:Malaise:1.06), (DOXEPIN:Malaise:0.09), (DRONABINOL:Malaise:0.42), (DUTASTE RIDE:Malaise:0.44), (EDOXABAN TOSYLATE:Malaise:0.06), (ELETRIPTAN HYDROBROMIDE:Malaise:0.54), (ENALAPRIL MALEATE:Malaise:0.68), (ERLOTINIB:Malaise:0.1), (FESOTERODINE FUMARATE:Malaise:0.26), (FLECAINIDE:Malaise:0.4), (FLUTICASONE:Malaise:0.68), (FLU TICASONE FUROATE:Malaise:0.42), (FLUTICASONE FUROATESALMETEROL XINAFOATE:Malaise:0.14), (FORMOTEROL FUMARATEMOMETASONE FUROATE:Malaise:0.11), (FULVESTRANT:Malaise:0.61), (GIMERACILOTERACILTEGAF UR:Malaise:0.38), (GLYCOPYRRONIUMINDACATEROL:Malaise:0.29), (HEPARIN SODIUM:Malaise:0.48), (HERBALSTURMERIC:Malaise:0.04), (HYDROCHLOROTHIAZID EIRBESARTAN:Malaise:0.02), (HYDROCHLOROTHIAZIDELISINOPRIL:Malaise:1.51), (H YDROCHLOROTHIAZIDELOSARTAN POTASSIUM:Malaise:0.14), (HYDROXYCHLOROQUINE DIPHOSPHATE:Malaise:0.33), (HYDROXYUREA:Malaise:0.33), (HYOSCYAMINE:Malaise:0 0.13), (IMIPRAMINE HYDROCHLORIDE:Malaise:0.34), (INDAPAMIDEPERINDOPRIL:Malaise:0.28), (INVESTI GATIONAL PRODUCT:Malaise:0.16), (ISOSORBIDE:Malaise:0.55), (LABETALOLLABETALOL HYDROCHLORIDE:Malaise:0.28), (LATANOPROST:Malaise:0.17), (LERCANIDIPINE:Malai se:0.3), (LERCANIDIPINE HYDROCHLORIDE:Malaise:0.1), (LINACLOTIDE:Malaise:0.08), (MEPOLIZUMAB:Malaise: 0.13), (MESNA:Malaise:0.37), (METHENAMINE HIPPURATE:Malaise:0.1), (METHYLPHENIDATE HYDROCHLORIDE:Malaise:0.52), (METHYLPREDNISOLONE ACETATE:Malaise:0.28), (METOLAZONE:Malaise:0.22), (MINOCYCLINE HYDROCHLORIDE:Malaise:0.1), (MOXIFLOXACIN:Malaise:0.09), (NABUMETONE:Malais e:0.01), (NALOXONE HYDROCHLORIDEOXYCODONE HYDROCHLORIDE:Malaise:0.45), (NIRAPARIB:Malaise:0.34), (OLODATEROL HYDROCHLORIDETIOTROPIUM BROMIDE MONOHYDRATE:Malaise:0.2), (OXYBUTYNIN CHLORIDE:Malaise:0.29), (PAMIDRONATE DISODIUM:Malaise:0.05), (PEGINTERFERON BETA-1A:Malaise:0.47), (PEMETREXED DISODIUM:Malaise:0.48), (PENICILLIN:Malaise:0.2), (PERINDOPRIL:Malaise:0.52), (PRAV ASTATINPRAVASTATIN SODIUM:Malaise:0.06), (PRIMIDONE:Malaise:0.94), (PROPRANOLOLPROPRANOLOL HYDROCHLORIDE:Malaise:0.05), (RALOXIFENE HYDROCHLORIDE:Malaise:0.1), (RIZATRIPTAN BENZOATE:Malaise:0.52), (ROPINIROLE HYDROCHLORIDE:Malaise:0.22), (SCOPOLAMINE:Malaise:0.39), (TERBUTALINE SULFATE:Malaise:0.29), (TIMOLOL:Malaise:0.07), (TINZAPARIN SODIUM:Malaise:0.35), (UMECLIDINIUM BROMIDEVILANTEROL TRIFENATATE:Malaise:0.31), (VANCOMYCIN HYDROCHLORIDE:Malaise:0.15), (VILAZODONE HYDROCHLORIDE:Malaise:0.04), (VITAMIN B:Malaise:0.72), (ZONISAMIDE:Malaise:0.47), (.ALPHA.-TOCOPHEROL ACETATE, DL-ASCORBIC ACIDCYANOCOBALAMINFLUORIDE IONFOLIC ACIDNIACINPYRIDOXINERIBOFLAVINTHIAMINEVITAMIN AVITAMIN D:Malignant melanoma:0.03), (ABOBOTULINUMTOXINA:Malignant melanoma:0.05), (ACEBUTOLOL HYDROCHLORIDE:Malignant melanoma:0.66), (ACETAMINOPHENCAFFEINE:Malignant melanoma:0.1), (ACETAMINOPHENPROPOXYPHENE HYDROCHLORIDE:Malignant melanoma:0.64), (ACETAMINOPHENPROPOXYPHENE NAPSYLATE:Malignant melanoma:0.12), (ACLIDINIUM BROMIDE:Malignant melanoma:0.29), (ADO-TRASTUZUMAB EMTANSINE:Malignant melanoma:1.25), (AFATINIB:Malignant melanoma:0.28), (AGALSIDASE BETA:Malignant melanoma:0.88), (ALBUTEROLIPRATROPIUM:Malignant melanoma:0.27), (AMANTADINE:Malignant melanoma:0.37), (AMIKACIN SULFATE:Malignant melanoma:0.22), (AMISULPRIDE:Malignant melanoma:0.39), (ANTIHISTAMINES NOS:Malignant melanoma:0.16), (ASPIRINDIHYDROXYALUMINUM AMINOACETATE ANHYDROUSMAGNESIUM CARBONATE:Malignant melanoma:0.09), (AZELASTINE HYDROCHLORIDE:Malignant melanoma:0.15), (AZOSEMIDE:Malignant melanoma:0.66), (BACITRACIN:Malignant melanoma:0.71), (BALSALAZIDE DISODIUM:Malignant melanoma:0.18), (BECLOMETHASONE:Malignant melanoma:0.01), (BECLOMETHASONE DIPROPIONATEFORMOTEROL:Malignant melanoma:0.15), (BENAZEPRILBENAZEPRIL HYDROCHLORIDE:Malignant melanoma:0.97), (BENDAMUSTINE:Malignant melanoma:0.18), (BERAPROST SODIUM:Malignant melanoma:0.22), (BETAMETHASONE DIPROPIONATECALCIPOTRIENECALCIPOTRIENE HYDRATE:Malignant melanoma:0.24), (BETHANECHOL CHLORIDE:Malignant melanoma:0.18), (BUDESONIDEFORMOTEROL:Malignant melanoma:0.02), (BUDESONIDEFORMOTEROL FUMARATE:Malignant melanoma:0.69), (BUPRENORPHINE HYDROCHLORIDE:Malignant melanoma:0.55), (CABAZITAXEL:Malignant melanoma:0.21), (CALCITONIN SALMON:Malignant melanoma:0.81), (CALCIUM CARBONATEERGOCALCIFEROLRETINOL:Malignant melanoma:0.13), (CALCIUM LACTATE:Malignant melanoma:0.13), (CANAGLIFLOZINMETFORMIN HYDROCHLORIDE:Malignant melanoma:1), (CARFILZOMIB:Malignant melanoma:1.29), (CARVEDILOL PHOSPHATE:Malignant melanoma:1.35), (CASANTHRANOLDOCUSATE SODIUM:Malignant melanoma:0.36), (CEFOTAXIMECEFOTAXIME SODIUM:Malignant melanoma:0.33), (CERTOLIZUMAB PEGOL:Malignant melanoma:1.71), (CERTOPARIN SODIUM:Malignant melanoma:0.24), (CETIRIZINE HYDROCHLORIDEPSEUDOEPHEDRINE HYDROCHLORIDE:Malignant melanoma:0.03), (CHLORAMBUCIL:Malignant melanoma:0.37), (CHLORDIAZEPOXIDE HYDROCHLORIDECLIDINIUM BROMIDE:Malignant melanoma:0.35), (CHLORHEXIDINE:Malignant melanoma:0.79), (CHLORPHENIRAMINEHYDROCODONE:Malignant melanoma:0.13), (CHONDROITIN SULFATE (BOVINE)GLUCOSAMINE HYDROCHLORIDE:Malignant melanoma:0.58), (CICLESONIDE:Malignant melanoma:0.14), (CIPROFIBRATE:Malignant melanoma:0.12), (CLOMIPRAMINE HYDROCHLORIDE:Malignant melanoma:0.38), (CLOZAPINE:Malignant melanoma:0.7), (CYPROHEPTADINE:Malignant melanoma:0.37), (DABIGATRAN:Malignant melanoma:1.1), (DACLIZUMAB:Malignant melanoma:1.15), (DANAZOL:Malignant melanoma:0.65), (DANTROLENE SODIUM:Malignant melanoma:0.07), (DAPAGLIFLOZIN PROPANEDIOL:Malignant melanoma:0.75), (DARATUMUMAB:Malignant melanoma:0.02), (DEFLAZACORT:Malignant melanoma:1.13), (DEXAMETHASONE ACETATE:Malignant melanoma:0.89), (EMPAGLIFLOZINLINAGLIPTIN:Malignant melanoma:0.69), (ENALAPRIL MALEATELERCANIDIPINE HYDROCHLORIDE:Malignant melanoma:0.02), (EPIRUBICIN:Malignant melanoma:0.54), (ERIBULIN:Malignant melanoma:0.57), (ERIBULIN MESYLATE:Malignant melanoma:0.05), (ESMOLOL HYDROCHLORIDE:Malignant melanoma:0.02), (ETRETINATE:Malignant melanoma:0.45), (FENOTEROL HYDROBROMIDE:Malignant melanoma:0.26), (FENOTEROL HYDROBROMIDEIPRATROPIUM BROMIDE:Malignant melanoma:0.49), (FERRIC CARBOXYMALTOSE:Malignant melanoma:0), (FERROUS SULFATEFOLIC ACID:Malignant melanoma:0.72), (FESOTERODINE FUMARATE:Malignant melanoma:1.48), (FISH OIL\OMEGA-3 FATTY ACIDS:Malignant melanoma:0.02), (FONDAPARINUX:Malignant melanoma:0.32), (FORMOTEROL FUMARATE DIHYDRATEMOMETASONE FUROATE:Malignant melanoma:0.73), (GIMERACILOTERACILTEGAFUR:Malignant melanoma:0.64), (GLYCOPYRROLATEINDACATEROL MALEATE:Malignant melanoma:0.04), (GLYCOPYRRONIUMINDACATEROL:Malignant melanoma:1.14), (GRANULOCYTE COLONY-STIMULATING FACTOR NOS:Malignant melanoma:0.17), (HERBALSTURMERIC:Malignant melanoma:0.78), (HYDROXYCHLOROQUINE DIPHOSPHATE:Malignant melanoma:0.65), (IDELALISIB:Malignant melanoma:0.54), (ILOPROST:Malignant melanoma:0.1), (IMIPRAMINE HYDROCHLORIDE:Malignant melanoma:0.82), (INFLIXIMAB-DYYB:Malignant melanoma:0.84), (IRINOTECAN:Malignant melanoma:0.3), (IRINOTECAN HYDROCHLORIDE:Malignant melanoma:0.76), (LACTASE:Malignant melanoma:0.71), (LANREOTIDE:Malignant melanoma:0.12), (LAPATINIB DITOSYLATE:Malignant melanoma:0.55), (LENOGRASTIM:Malignant melanoma:0.18), (LEVOLEUCOVORIN CALCIUM:Malignant melanoma:0.42), (LIOTHYRONINE:Malignant melanoma:0.1), (LIOTHYRONINE SODIUM:Malignant melanoma:0.3), (LITHIUM CARBONATE:Malignant melanoma:1.12), (LOMITAPIDE MESYLATE:Malignant melanoma:0.26), (LUTEIN:Malignant melanoma:0.46), (MELPERONE:Malignant melanoma:0.24), (MERCAPTOPURINE:Malignant melanoma:0.08), (MESNA:Malignant melanoma:0.04), (METAXALONE:Malignant melanoma:1.18), (METFORMIN HYDROCHLORIDEVILDAGLIPTIN:Malignant melanoma:0.8), (METFORMIN PAMOATE:Malignant melanoma:0.25), (METHENOLONE ENANTHATE:Malignant melanoma:0.28), (METHYLPREDNISOLONE ACETATE:Malignant melanoma:1.73), (METOLAZONE:Malignant melanoma:0.63), (MIGLITOL:Malignant melanoma:0.14), (MODAFINIL:Malignant melanoma:0.41), (MORPHINE SULFATENALTREXONE HYDROCHLORIDE:Malignant melanoma:0.43), (NADOLOL:Malignant melanoma:0.92), (NINTEDANIB:Malignant melanoma:0.71), (NIRAPARIB:Malignant melanoma:0.81), (NORTRIPTYLINE HYDROCHLORIDE:Malignant melanoma:0.02), (OCTREOTIDE:Malignant melanoma:0.2), (OLAPARIB:Malignant melanoma:0.55), (OLODATEROL HYDROCHLORIDETIOTROPIUM BROMIDE MONOHYDRATE:Malignant melanoma:0.75), (ONABOTULINUMTOXINA:Malignant melanoma:0.47), (ORPHENADRINE:Malignant melanoma:0.27), (OSIMERTINIB:Malignant melanoma:0.53), (PANITUMUMAB:Malignant melanoma:0.16), (PATIROMER:Malignant melanoma:0.12), (PEGINTERFERON BETA-1A:Malignant melanoma:0.41), (PENICILLIN G:Malignant melanoma:0.26), (PENICILLIN V POTASSIUM:Malignant melanoma:0.01), (PENTAMIDINE ISETHIONATE:Malignant melanoma:0.44), (PERTUZUMAB:Malignant melanoma:0.21), (PIMECROLIMUS:Malignant melanoma:0.38), (PIMOBENDAN:Malignant melanoma:0.08), (POLYETHYLENE GLYCOL 3350\POTASSIUM CHLORIDESODIUM BICARBONATESODIUM CHLORIDESODIUM SULFATE ANHYDROUS:Malignant melanoma:0.22), (PONATINIB:Malignant melanoma:1), (PORFIMER SODIUM:Malignant melanoma:0.02), (PRAMIPEXOLE DIHYDROCHLORIDE:Malignant melanoma:1.09), (PRAZOSIN:Malignant melanoma:0.18), (PRAZOSIN HYDROCHLORIDE:Malignant melanoma:0.19), (PYRIDOXAL:Malignant melanoma:0.03), (RAMUCIRUMAB:Malignant melanoma:1.02), (REGORAFENIB:Malignant melanoma:1.66), (RIFAXIMIN:Malignant melanoma:0.15), (RILPIVIRINE HYDROCHLORIDE:Malignant melanoma:0.04), (RIVASTIGMINE:Malignant melanoma:0.37), (RIVASTIGMINE TARTRATE:Malignant melanoma:0.6), (RIZATRIPTAN BENZOATE:Malignant melanoma:0.8), (ROFLUMILAST:Malignant melanoma:0.98), (SARPOGRELATE:Malignant melanoma:0.43), (SECUKINUMAB:Malignant melanoma:0.84), (SODIUM CITRATE:Malignant melanoma:0.14), (SODIUM OXYBATE:Malignant melanoma:0.78), (STARCH, WHEAT:Malignant melanoma:0.01), (SUFENTANIL CITRATE:Malignant melanoma:0.07), (SULINDAC:Malignant melanoma:0.04), (SUMATRIPTANSUMATRIPTAN SUCCINATE:Malignant melanoma:0.82), (TAMOXIFEN CITRATE:Malignant melanoma:0.03), (TEDUGLUTIDEWATER:Malignant melanoma:0), (TERAZOSIN HYDROCHLORIDE:Malignant melanoma:0.59), (TERBINAFINE HYDROCHLORIDE:Malignant melanoma:1.01), (TESTOSTERONE UNDECANOATE:Malignant melanoma:0.53), (THIOCTIC ACID:Malignant melanoma:0.04), (TICLOPIDINE:Malignant melanoma:0.37), (TIMOLOL MALEATETRAVOPROST:Malignant melanoma:0.2), (TIOTROPIUM:Malignant melanoma:1.08), (TIPIRACIL HYDROCHLORIDETRIFLURIDINE:Malignant melanoma:0.67), (TRIPTORELIN PAMOATE:Malignant melanoma:0), (UMECLIDINIUM BROMIDE:Malignant melanoma:0.41), (UMECLIDINIUM BROMIDEVILANTEROL TRIFENATATE:Malignant melanoma:0.82), (ZAFIRLUKAST:Malignant melanoma:0.02), (ZINC:Malignant melanoma:0.83), (ZOLMITRIPTAN:Malignant melanoma:0.25), (DASATINIB:Malignant neoplasm progression:0.17), (IMATINIB MESYLATE:Malignant neoplasm progression:0.08), (LEUCOVORIN CALCIUM:Malignant neoplasm progression:0.21), (NILOTINIB:Malignant neoplasm progression:0.05), (RADIATION THERAPY:Malignant neoplasm progression:0.03), (BUDESONIDE:Malignant peritoneal neoplasm:0.13), (BUMETANIDE:Malignant peritoneal neoplasm:0.15), (CALCIUM CARBONATECHOLECALCIFEROL:Malignant peritoneal neoplasm:0.3), (CETUXIMAB:Malignant peritoneal neoplasm:0.48), (CLINDAMYCINCLINDAMYCIN PHOSPHATE:Malignant peritoneal neoplasm:0.01), (DEXLANSOPRAZOLE:Malignant peritoneal neoplasm:0.31), (EPIRUBICIN:Malignant peritoneal neoplasm:0.26), (FERROUS SULFATEFERROUS SULFATE, DRIED:Malignant peritoneal neoplasm:0.3), (IBRUTINIB:Malignant peritoneal neoplasm:0.74), (ILOPROST:Malignant peritoneal neoplasm:0.04), (LEVOLEUCOVORIN CALCIUM:Malignant peritoneal neoplasm:0.19), (NILOTINIB:Malignant peritoneal neoplasm:0.05), (PRAVASTATIN SODIUM:Malignant peritoneal neoplasm:0.03), (TAMSULOSIN:Malignant peritoneal neoplasm:0.23), (TORSEMIDE:Malignant peritoneal neoplasm:0.04), (BUMETANIDE:Malignant respiratory tract neoplasm:0.15), (CITALOPRAM HYDROBROMIDE:Malignant respiratory tract neoplasm:0.02), (DEXLANSOPRAZOLE:Malignant respiratory tract neoplasm:0.1), (IBRUTINIB:Malignant respiratory tract neoplasm:0.07), (BISOPROLOL:Malignant urinary tract neoplasm:0.07), (BUMETANIDE:Malignant urinary tract neoplasm:0.41), (CALCIUM CARBONATECHOLECALCIFEROL:Malignant urinary tract neoplasm:0.23), (CLONAZEPAM:Malignant urinary tract neoplasm:0.13), (DEXLANSOPRAZOLE:Malignant urinary tract neoplasm:0.79), (EPINEPHRINE:Malignant urinary tract neoplasm:0.03), (EVOLOCUMAB:Malignant urinary tract neoplasm:0.12), (FERROUS SULFATEFERROUS SULFATE, DRIED:Malignant urinary tract neoplasm:0.27), (FLUTICASONE PROPIONATE:Malignant urinary tract neoplasm:0.66), (IBRUTINIB:Malignant urinary tract neoplasm:0.45), (INSULIN ASPART:Malignant urinary tract neoplasm:0.15), (NAPROXEN:Malignant urinary tract neoplasm:0.5), (TORSEMIDE:Malignant urinary tract neoplasm:0.39), (TRAZODONE HYDROCHLORIDE:Malignant urinary tract neoplasm:0.1), (ACETAMINOPHENCODEINE PHOSPHATE:Malnutrition:0.11), (ANASTROZOLE:Malnutrition:0.13), (ATENOLOL:Malnutri tion:0.07), (BUDESONIDE:Malnutrition:0.74), (BUMETANIDE:Malnutrition:0.44), (BUPROPI ON HYDROCHLORIDE:Malnutrition:0.35), (CELECOXIB:Malnutrition:0.59), (CEPHALEXIN:Mal nutrition:0.5), (CLINDAMYCINCLINDAMYCIN PHOSPHATE:Malnutrition:0.59), (CLOTRIMAZOLE:Malnutrition:0.07), (DILTIAZEM:Malnut rition:0.06), (DIVALPROEX SODIUM:Malnutrition:0.4), (DULOXETINE HYDROCHLORIDE:Malnutrition:0.21), (IRON:Malnutrition:0.11), (LEDIPASVIRSOFOSBUV IR:Malnutrition:0.15), (MICAFUNGIN:Malnutrition:0.09), (NIFEDIPINE:Malnutrition:0.23), (NI LOTINIB:Malnutrition:0.34), (PANITUMUMAB:Malnutrition:0.04), (SACUBITRILVALSART AN:Malnutrition:0.09), (THALIDOMIDE:Malnutrition:0.27), (TOCILIZUMAB:Malnutrition:0.0 4), (TOPIRAMATE:Malnutrition:0.4), (TRAZODONE HYDROCHLORIDE:Malnutrition:0.57), (WARFARIN SODIUM:Malnutrition:0.23), (AMIODARONE HYDROCHLORIDE:Mania:0.1), (AMOXICILLIN:Mania:0.36), (ANASTROZOLE:Mania:0.04), (BUMETANIDE:Mania:0.02), (BUPRENORPHINENALOXONE:Mania:0.08), (CALCIUM CARBONATECHOLECALCIFEROL:Mania:0.38), (CARVEDILOL:Mania:0.28), (CERTOLIZ UMAB PEGOL:Mania:0.1), (CETIRIZINE HYDROCHLORIDE:Mania:0.35), (CHOLESTYRAMINE:Mania:0.04), (DEXLANSOPRAZOL E:Mania:0.44), (DUTASTERIDE:Mania:0.07), (ELETRIPTAN HYDROBROMIDE:Mania:0.15), (EVEROLIMUS:Mania:0.32), (EVOLOCUMAB:Mania:0.22), (FERROUS SULFATEFERROUS SULFATE, DRIED:Mania:0.47), (FLUPHENAZINE:Mania:0), (GLIPIZIDE:Mania:0.02), (INDOMETHACI N:Mania:0. 11), (INSULIN LISPRO:Mania:0.16), (IRON:Mania:0.24), (LABETALOLLABETALOL HYDROCHLORIDE:Mania:0.15), (LACOSAMIDE:Mania:0.03), (NITROFURANTOIN:Mania: 0.1), (PRAMIPEXOLE DIHYDROCHLORIDE:Mania:0.71), (PROBIOTICS NOS:Mania:0.08), (SACUBITRILVALSARTAN:Mania:0.14), (SECUKINUMAB:Mania:0.17), (TERIFLUNOMIDE:Mania:0.51), (TIOTROPIUM BROMIDE MONOHYDRATE:Mania:0.35), (VARENICLINE TARTRATE:Mania:1.38), (VERAPAMIL HYDROCHLORIDE:Mania:0.49), (ZUCLOPENTHIXOL DECANOATE:Mania:0.03), (.ALPHA.1-PROTEINASE INHIBITOR HUMAN:Mantle cell lymphoma:0.08), (ACENOCOUMAROL:Mantle cell lymphoma:0.5), (ACETAMINOPHENPROPOXYPHENE HYDROCHLORIDE:Mantle cell lymphoma:0.02), (ACETAZOLAMIDE:Mantle cell lymphoma:0.69), (ADO-TRASTUZUMAB EMTANSINE:Mantle cell lymphoma:0.19), (ALENDRONIC ACID:Mantle cell lymphoma:0.57), (ALFACALCIDOL:Mantle cell lymphoma:0.57), (ALISKIREN HEMIFUMARATE:Mantle cell lymphoma:0.19), (AMISULPRIDE:Mantle cell lymphoma:0.08), (AMITRIPTYLINE HYDROCHLORIDE:Mantle cell lymphoma:0.66), (AMLODIPINE BESYLATEHYDROCHLOROTHIAZIDEOLMESARTAN MEDOXOMIL:Mantle cell lymphoma:0.26), (AMLODIPINEVALSARTAN:Mantle cell lymphoma:0.3), (ARIPIPRAZOLE:Mantle cell lymphoma:0.2),(ARMODAFINIL:Mantle cell lymphoma:0.85), (AXITINIB:Mantle cell lymphoma:0.06), (BLINATUMOMAB:Mantle cell lymphoma:0.6), (BUPRENORPHINE HYDROCHLORIDENALOXONE HYDROCHLORIDE:Mantle cell lymphoma:0.03), (CALCITONIN:Mantle cell lymphoma:0.36), (CALCITONIN SALMON:Mantle cell lymphoma:0.35), (CANAKINUMAB:Mantle cell lymphoma:0.28), (CANDESARTAN CILEXETILHYDROCHLOROTHIAZIDE:Mantle cell lymphoma:1.42), (CEFTAZIDIME:Mantle cell lymphoma:0.02), (CERTOLIZUMAB PEGOL:Mantle cell lymphoma:0.94), (CHLORHEXIDINE:Mantle cell lymphoma:0.3), (CHONDROITIN SULFATE (BOVINE)GLUCOSAMINE HYDROCHLORIDE:Mantle cell lymphoma:0.38), (CICLESONIDE:Mantle cell lymphoma:0.44), (CILASTATIN SODIUMIMIPENEM:Mantle cell lymphoma:0.9), (CIMETIDINE:Mantle cell lymphoma:0.87), (CINACALCET HYDROCHLORIDE:Mantle cell lymphoma:0.05), (CLINDAMYCIN PHOSPHATE:Mantle cell lymphoma:0.11), (CLOZAPINE:Mantle cell lymphoma:0.75), (CORTISONEHYDROCORTISONE:Mantle cell lymphoma:0.81), (CRIZOTINIB:Mantle cell lymphoma:0.11), (CYPROHEPTADINE:Mantle cell lymphoma:0.01), (DACLIZUMAB:Mantle cell lymphoma:0.69), (DANAZOL:Mantle cell lymphoma:0.57), (DAPAGLIFLOZIN PROPANEDIOL:Mantle cell lymphoma:0.13), (DEFLAZACORT:Mantle cell lymphoma:0.14), (DEVICE:Mantle cell lymphoma:0.2), (DIGITOXIN:Mantle cell lymphoma:0.17), (DIMENHYDRINATE:Mantle cell lymphoma:0.59), (ECONAZOLE:Mantle cell lymphoma:0.52), (EDOXABAN TOSYLATE:Mantle cell lymphoma:0.59), (ELETRIPTAN HYDROBROMIDE:Mantle cell lymphoma:0.67), (ELOTUZUMAB:Mantle cell lymphoma:0.25), (EPIRUBICIN:Mantle cell lymphoma:0.1), (ESTROGENS, CONJUGATEDMEDROXYPROGESTERONE ACETATE:Mantle cell lymphoma:0.26), (EVOLOCUMAB:Mantle cell lymphoma:0.25), (FEXOFENADINEPSEUDOEPHEDRINE:Mantle cell lymphoma:0.06), (FLUDROCORTISONE ACETATE:Mantle cell lymphoma:0.19), (FLUOCINONIDE:Mantle cell lymphoma:0.15), (FLUTICASONE FUROATE:Mantle cell lymphoma:0.55), (FORMOTEROL:Mantle cell lymphoma:0.25), (FORMOTEROL FUMARATEMOMETASONE FUROATE:Mantle cell lymphoma:0.13), (FUSIDIC ACID:Mantle cell lymphoma:0.4), (GLYCOPYRROLATE:Mantle cell lymphoma:0.13), (GLYCOPYRRONIUMINDACATEROL:Mantle cell lymphoma:0.96), (HERBALS:Mantle cell lymphoma:0.16), (HYDROCHLOROTHIAZIDETELMISARTAN:Mantle cell lymphoma:0.56), (HYDROXYZINE PAMOATE:Mantle cell lymphoma:1.19), (HYOSCYAMINE:Mantle cell lymphoma:0.49), (IDARUBICIN:Mantle cell lymphoma:0.01), (ILOPROST:Mantle cell lymphoma:0.03), (IMIPRAMINE HYDROCHLORIDE:Mantle cell lymphoma:0.11), (INDACATEROL:Mantle cell lymphoma:0.26), (IXAZOMIB:Mantle cell lymphoma:0.55), (KETOROLACKETOROLAC TROMETHAMINE:Mantle cell lymphoma:0.18), (LEDIPASVIRSOFOSBUVIR:Mantle cell lymphoma:0.73), (LERCANIDIPINE HYDROCHLORIDE:Mantle cell lymphoma:1.03), (MEPOLIZUMAB:Mantle cell lymphoma:0.05), (METHYLPREDNISOLONE ACETATE:Mantle cell lymphoma:0.29), (MICAFUNGIN:Mantle cell lymphoma:0.42), (MYCOPHENOLATE MOFETILMYCOPHENOLATE MOFETIL HYDROCHLORIDE:Mantle cell lymphoma:0.09), (NADROPARIN CALCIUM:Mantle cell lymphoma:0.28), (NALOXONE HYDROCHLORIDEOXYCODONE HYDROCHLORIDE:Mantle cell lymphoma:1.28), (NATEGLINIDE:Mantle cell lymphoma:0.35), (NILOTINIB:Mantle cell lymphoma:1.85), (NINTEDANIB:Mantle cell lymphoma:0.54), (OLODATEROL HYDROCHLORIDETIOTROPIUM BROMIDE MONOHYDRATE:Mantle cell lymphoma:0.62), (OXCARBAZEPINE:Mantle cell lymphoma:0.97), (PAMIDRONATE DISODIUM:Mantle cell lymphoma:0.21), (PAZOPANIB HYDROCHLORIDE:Mantle cell lymphoma:0.72), (PEGINTERFERON BETA-1A:Mantle cell lymphoma:0.08), (PEMETREXED:Mantle cell lymphoma:0.1), (PEMETREXED DISODIUM:Mantle cell lymphoma:0.53), (PENTOXIFYLLINE:Mantle cell lymphoma:0.16), (PONATINIB:Mantle cell lymphoma:1.14), (PRAZOSIN:Mantle cell lymphoma:0.15), (PREDNISOLONE SODIUM PHOSPHATE:Mantle cell lymphoma:0.02), (PYRIDOXINE:Mantle cell lymphoma:0.35), (SACUBITRILVALSARTAN:Mantle cell lymphoma:0.82), (SALMETEROL:Mantle cell lymphoma:0.35), (SALMETEROL XINAFOATE:Mantle cell lymphoma:0.1), (SODIUM FLUORIDE:Mantle cell lymphoma:0.04), (SODIUM OXYBATE:Mantle cell lymphoma:0.95), (SUMATRIPTANSUMATRIPTAN SUCCINATE:Mantle cell lymphoma:0.8), (TOLTERODINE:Mantle cell lymphoma:0), (TROSPIUM CHLORIDE:Mantle cell lymphoma:0.2), (VALGANCICLOVIR HYDROCHLORIDE:Mantle cell lymphoma:0.4), (VILAZODONE HYDROCHLORIDE:Mantle cell lymphoma:0.03), (ZAFIRLUKAST:Mantle cell lymphoma:0.05), (DOLUTEGRAVIR:Matemal exposure timing unspecified:0.43), (NAPROXEN:Maternal exposure timing unspecified:0.05), (OXCARBAZEPINE:Maternal exposure timing unspecified:0.77), (ANASTROZOLE:Medical diet:0.09), (BUPROPION HYDROCHLORIDE:Medical diet:1. 03), (BUSPIRONE HYDROCHLORIDE:Medical diet:0.21), (CIPROFLOXACIN HYDROCHLORIDE:Medical diet:0.37), (DEXLANSOPRAZOLE:Medical diet:0.9), (DICLOFENAC SODIUM:Medical diet:0.15), (DILTIAZEM:Medical diet:0.4), (DILTIAZEM HYDROCHLORIDE:Medical diet:0.57), (DIPHENHYDRAMINE:Medical diet:0.27), (DOCUSATE SODIUM:Medical diet:0.46), (ESZOPICLONE:Medical diet:0.08), (FERROUS SULFATE:Medical diet:0.13), (IBRUTINIB:Medical diet:0.56), (LISDEXAMFETAMINE DIMESYLATE:Medical diet:0.14), (MELATONIN:Medical diet:0.22), (PRAVASTATIN SODIUM:Medical diet:0.05), (SACUBITRILVALSARTAN:Medical diet:0.02), (TAMSULOSIN HYDROCHLORIDE:Medical diet:0.02), (TERIFLUNOMIDE:Medical diet:0.42), (TIZANIDINE HYDROCHLORIDE:Medical diet:0.03), (VERAPAMIL HYDROCHLORIDE:Medical diet:0.35), (ACETAMINOPHENCODEINE PHOSPHATE:Medication dilution:0.72),(ARMODAFINIL:Medication dilution:0.19), (BUPRENORPHINE HYDROCHLORIDENALOXONE HYDROCHLORIDE:Medication dilution:0.15), (CALCIUM ACETATE:Medication dilution:0.04), (CALCIUM CARBONATECHOLECALCIFEROL:Medication dilution:0.92), (CANDESARTAN:Medication dilution:0.05), (CARFILZOMIB:Medication dilution:0.09), (CHOLESTYRAMINE:Medication dilution:0.35), (CLOZAPINE:Medication dilution:0), (CODEINE:Medication dilution:0.02), (DASATINIB:Medication dilution:0.18), (DEXLANSOPRAZOLE:Medication dilution:0.76), (DICLOFENAC:Medication dilution:0.3), (DICLOFENAC SODIUM:Medication dilution:0.2), (DIMENHYDRINATE:Medication dilution:0.11), (DOCUSATE:Medication dilution:0.06), (ENOXAPARIN:Medication dilution:0.61), (ESZOPICLONE:Medication dilution:0.52), (EVOLOCUMAB:Medication dilution:0.25), (FLECAINIDE:Medication dilution:0.05), (GLIPIZIDE:Medication dilution:0.06), (HYDROCHLOROTHIAZIDELISINOPRIL:Medication dilution:0.27), (HYDROXYZINE PAMOATE:Medication dilution:0.1), (IBRUTINIB:Medication dilution:0.7), (ILOPROST:Medication dilution:0.17), (IMIPENEM:Medication dilution:0.08), (IPRATROPIUM:Medication dilution:0.2), (IRINOTECAN:Medication dilution:0.56), (LEVALBUTEROL HYDROCHLORIDE:Medication dilution:0.05), (LITHIUM CARBONATE:Medication dilution:0.08), (MOXIFLOXACIN:Medication dilution:0.03), (MOXIFLOXACIN HYDROCHLORIDE:Medication dilution:0.28), (NILOTINIB:Medication dilution:1.11), (NITROFURANTOIN:Medication dilution:0.43), (NYSTATIN:Medication dilution:0.4), (OXCARBAZEPINE:Medication dilution:0.13), (POSACONAZOLE:Medication dilution:0.28), (PRAMIPEXOLE DIHYDROCHLORIDE:Medication dilution:0.06), (PROBIOTICS NOS:Medication dilution:0.23), (RANOLAZINE:Medication dilution:0.07), (RISEDRONATE SODIUM:Medication dilution:0.14), (SACUBITRILVALSARTAN:Medication dilution:0.3), (SODIUM OXYBATE:Medication dilution:0.11), (TELMISARTAN:Medication dilution:0.32), (TERIFLUNOMIDE:Medication dilution:0.43), (THALIDOMIDE:Medication dilution:0.53), (TIZANIDINE HYDROCHLORIDE:Medication dilution:0.21), (TOCILIZUMAB:Medication dilution:0.28), (VALGANCICLOVIR HYDROCHLORIDE:Medication dilution:0.2), (VARENICLINE TARTRATE:Medication dilution:0.35), (.ALPHA.-TOCOPHEROL ACETATE, DL-ASCORBIC ACIDCYANOCOBALAMINFLUORIDE IONFOLIC ACIDNIACINPYRIDOXINERIBOFLAVINTHIAMINEVITAMIN AVITAMIN D:Memory impairment:0.01), (ACETAMINOPHENOXYCODONE HYDROCHLORIDEOXYCODONE TEREPHTHALATE:Memory impairment:0.4), (ACETAMINOPHENOXYCODONEOXYCODONE HYDROCHLORIDE:Memory impairment:0.38), (ALCOHOL:Memory impairment:0.26), (ALENDRONIC ACID:Memory impairment:0.11), (ALFACALCIDOL:Memory impairment:0), (ALISKIREN HEMIFUMARATE:Memory impairment:0.06), (AMISULPRIDE:Memory impairment:0.1), (AZITHROMYCIN DIHYDRATE:Memory impairment:0.16), (BECLOMETHASONE DIPROPIONATE:Memory impairment:0.39), (BIFIDOBACTERIUM LONGUM SUBSP. INFANTIS:Memory impairment:0.21), (BRINZOLAMIDE:Memory impairment:0.01), (CALCIUM ACETATE:Memory impairment:0.49), (CANDESARTAN:Memory impairment:0.94), (CERTOLIZUMAB PEGOL:Memory impairment:1.05), (CETIRIZINE HYDROCHLORIDEPSEUDOEPHEDRINE HYDROCHLORIDE:Memory impairment:0.07), (CHLORDIAZEPOXIDE HYDROCHLORIDECLIDINIUM BROMIDE:Memory impairment:0.12), (CHOLESTYRAMINE:Memory impairment:0.86), (CLINDAMYCIN:Memory impairment:0.1), (CORTICOTROPIN:Memory impairment:0.28), (DACLIZUMAB:Memory impairment:0.42), (DASATINIB:Memory impairment:0.97), (DIMENHYDRINATE:Memory impairment:0.2), (DOXEPIN HYDROCHLORIDE:Memory impairment:0.26), (DUTASTERIDETAMSULOSIN HYDROCHLORIDE:Memory impairment:0.1), (EDOXABAN TOSYLATE:Memory impairment:0.21), (ELETRIPTAN HYDROBROMIDE:Memory impairment:1.25), (EMPAGLIFLOZINLINAGLIPTIN:Memory impairment:0.1 1), (ENOXAPARIN SODIUM:Memory impairment:0.3), (ERYTHROPOIETIN:Memory impairment:0.21), (ESTROGENS:Memory impairment:0.1), (ETORICOXIB:Memory impairment:0.17), (FEBUXOSTAT:Memory impairment:0.23), (FESOTERODINE FUMARATE:Memory impairment:1.01), (FLUTICASONE:Memory impairment:0.71), (FLUTICASONE FUROATE:Memory impairment:0.31), (GLYCOPYRRONIUMINDACATEROL:Memory impairment:0.28), (GUANFACINE:Memory impairment:0.14), (GUANFACINE HYDROCHLORIDE:Memory impairment:0.14), (HEPARIN SODIUM:Memory impairment:0.51), (HYDROCHLOROTHIAZIDEIRBESARTAN:Memory impairment:0.4), (HYDROCHLOROTHIAZIDETELMISARTAN:Memory impairment:0.19), (HYDROMORPHONE HYDROCHLORIDE:Memory impairment:1.13), (HYDROXYCHLOROQUINE DIPHOSPHATE:Memory impairment:0.11), (ILOPROST:Memory impairment:0.02), (IMATINIB MESYLATE:Memory impairment:0.42), (INDAPAMIDEPERINDOPRIL:Memory impairment:0.05), (INSULIN GLULISINE:Memory impairment:0.12), (IPRATROPIUM:Memory impairment:0.81), (LERCANIDIPINE HYDROCHLORIDE:Memory impairment:0.21), (LEVOMEFOLATE CALCIUM:Memory impairment:0.26), (LITHIUM CARBONATE:Memory impairment:1.02), (LOMITAPIDE MESYLATE:Memory impairment:0.22), (MEDROXYPROGESTERONE ACETATE:Memory impairment:0.18), (METAXALONE:Memory impairment:1.71), (METHYLPREDNISOLONE ACETATE:Memory impairment:1.04), (NALOXONE HYDROCHLORIDEOXYCODONE HYDROCHLORIDE:Memory impairment:0.46), (NALTREXONE:Memory impairment:0.41), (OXAZEPAM:Memory impairment:0.08), (PANTOPRAZOLE MAGNESIUM:Memory impairment:0.89), (PAROXETINE:Memory impairment:0.89), (PAZOPANIB HYDROCHLORIDE:Memory impairment:0), (PEGINTERFERON BETA-1A:Memory impairment:0.41), (PENICILLIN:Memory impairment:0.43), (PERINDOPRIL:Memory impairment:0.28), (PIROXICAM:Memory impairment:0.09), (PONATINIB:Memory impairment:0.16), (PRAZOSIN HYDROCHLORIDE:Memory impairment:0.07), (PROGESTERONE:Memory impairment:0.31), (PYRIDOXINE:Memory impairment:0.19), (QUINAPRILQUINAPRIL HYDROCHLORIDE:Memory impairment:0.06), (RABEPRAZOLE:Memory impairment:0.5), (RIZATRIPTAN BENZOATE:Memory impairment:0.87), (ROFLUMILAST:Memory impairment:0.42), (SCOPOLAMINE:Memory impairment:0.59), (SECUKINUMAB:Memory impairment:0.41), (SELEGILINE:Memory impairment:0.27), (SELEGILINE HYDROCHLORIDE:Memory impairment:0.08), (SODIUM BICARBONATE:Memory impairment:0.06), (SUCRALFATE:Memory impairment:0.78), (TERBINAFINE HYDROCHLORIDE:Memory impairment:0.16), (TERBUTALINE SULFATE:Memory impairment:0.02), (TETRACYCLINE:Memory impairment:0.35), (UMECLIDINIUM BROMIDEVILANTEROL TRIFENATATE:Memory impairment:0.79), (ACETAMINOPHENCODEINE PHOSPHATE:Meniere's disease:0.24), (AMIODARONE:Meniere's disease:0.38),(ARMODAFINIL:Meniere's disease:0.03), (BUSPIRONE HYDROCHLORIDE:Meniere's disease:0.32), (CALCIUM CARBONATECHOLECALCIFEROL:Meniere's disease:0.65), (CORTISONEHYDROCORTISONE:Meniere's disease:0.3), (DIPHENHYDRAMINE:Meniere's disease:0.05), (DOCUSATE SODIUM:Meniere's disease:0.62), (ENOXAPARIN:Meniere's disease:0.03), (ESZOPICLONE:Meniere's disease:0.04), (EVEROLIMUS:Meniere's disease:0.02), (EVOLOCUMAB:Meniere's disease:0.94), (FERROUS SULFATEFERROUS SULFATE, DRIED:Meniere's disease:0.76), (HEPARIN SODIUM:Meniere's disease:0.09), (HYDROCHLOROTHIAZIDELISINOPRIL:Meniere's disease:0.06), (ILOPROST:Meniere's disease:0.12), (LENALIDOMIDE:Meniere's disease:0.07), (METAXALONE:Meniere's disease:0.02), (NILOTINIB:Meniere's disease:0.09), (OXCARBAZEPINE:Meniere's disease:0.34), (PAROXETINE:Meniere's disease:0.12), (PRAMIPEXOLE DIHYDROCHLORIDE:Meniere's disease:0.08), (PRIMIDONE:Meniere's disease:0.09), (RANOLAZINE:Meniere's disease:0.14), (SECUKINUMAB:Meniere's disease:0.23), (TERIFLUNOMIDE:Meniere's disease:0.83), (TOCILIZUMAB:Meniere's disease:0.26), (VITAMIN B:Meniere's disease:0.07), (GADOVERSETAMIDE:Meningioma:0.3), (CLOBAZAM:Meningitis:0.11), (ABA CAVIR:Meningitis cryptococcal:0.17), (EMTRICITABINETENOFOVIR:Meningitis cryptococcal:0.06), (MARAVIROC:Meningitis cryptococcal:0.09), (ETHAMBUTOL HYDROCHLORIDE:Meningitis histoplasma:0.69), (ETHAMBUTOL HYDROCHLORIDE:Meningitis viral:0.73), (AMANTADINE:Menopausal symptoms:0.11), (AMIODARONE:Menopausal symptoms:0.19), (BISOPROLOL FUMARATE:Menopausal symptoms:0.4), (BUMETANIDE:Menopausal symptoms:0.12), (BUPRENORPHINE HYDROCHLORIDENALOXONE HYDROCHLORIDE:Menopausal symptoms:0.38), (CANDESARTAN CILEXETIL:Menopausal symptoms:0.61), (CANDESARTAN CILEXETILHYDROCHLOROTHIAZIDE:Menopausal symptoms:0.17), (CARVEDILOL PHOSPHATE:Menopausal symptoms:0.02), (CHOLESTYRAMINE:Menopausal symptoms:0.33), (CIPROFLOXACIN HYDROCHLORIDE:Menopausal symptoms:0.14), (CODEINE:Menopausal symptoms:0.18), (DASATINIB:Menopausal symptoms:0.16), (DOCETAXEL:Menopausal symptoms:0.23), (DRONABINOL:Menopausal symptoms:0.01), (DUTASTERIDE:Menopausal symptoms:0.21), (ELETRIPTAN HYDROBROMIDE:Menopausal symptoms:1.07), (ENOXAPARIN:Menopausal symptoms:0.45), (ESOMEPRAZOLE:Menopausal symptoms:0.22), (ESTROGENS:Menopausal symptoms:0.17), (ETHINYL ESTRADIOLNORGESTIMATE:Menopausal symptoms:0.62), (EVOLOCUMAB:Menopausal symptoms:0.88), (HYDROXYCHLOROQUINE DIPHOSPHATE:Menopausal symptoms:0.5), (IBRUTINIB:Menopausal symptoms:1.02), (INSULIN DETEMIR:Menopausal symptoms:0.09), (ISOPROPYL ALCOHOLLEUPROLIDE ACETATENORETHINDRONE ACETATE:Menopausal symptoms:0.24), (ISOSORBIDE:Menopausal symptoms:0), (LABETALOLLABETALOL HYDROCHLORIDE:Menopausal symptoms:0.27), (LENALIDOMIDE:Menopausal symptoms:0.02), (LISDEXAMFETAMINE DIMESYLATE:Menopausal symptoms:0.58), (LITHIUM CARBONATE:Menopausal symptoms:0.3), (METRONIDAZOLE:Menopausal symptoms:1.29), (MORPHINE:Menopausal symptoms:1.03), (MOXIFLOXACIN HYDROCHLORIDE:Menopausal symptoms:0.02), (NIACIN:Menopausal symptoms:0.17), (OLANZAPINE:Menopausal symptoms:0.7), (OXCARBAZEPINE:Menopausal symptoms:1.46), (PEGINTERFERON BETA-1A:Menopausal symptoms:0.07), (PENICILLIN:Menopausal symptoms:0.55), (PENTOSAN POLYSULFATE SODIUM:Menopausal symptoms:0.14), (PHENYTOIN:Menopausal symptoms:0.15), (POLYETHYLENE GLYCOLS:Menopausal symptoms:0.66), (PRIMIDONE:Menopausal symptoms:0.62), (PROMETHAZINEPROMETHAZINE HYDROCHLORIDE:Menopausal symptoms:0.45), (RANOLAZINE:Menopausal symptoms:0.17), (SACUBITRILVALSARTAN:Menopausal symptoms:0.37), (TAMSULOSIN HYDROCHLORIDE:Menopausal symptoms:0.31), (TIOTROPIUM BROMIDE MONOHYDRATE:Menopausal symptoms:0.56), (TOCILIZUMAB:Menopausal symptoms:0.44), (UMECLIDINIUM BROMIDEVILANTEROL TRIFENATATE:Menopausal symptoms:0.07), (VITAMIN B:Menopausal symptoms:0.64), (.ALPHA.1-PROTEINASE INHIBITOR HUMAN:Menopause:0.03), (ACETAMINOPHENOXYCODONE HYDROCHLORIDEOXYCODONE TEREPHTHALATE:Menopause:0.29), (ACETAZOLAMIDE:Menopause:0.7), (ALCOHOL:Me nopause:0.11), (ALENDRONIC ACID:Menopause:0.07), (AMANTADINE:Menopause:0.34), (AMIODARONE:Menopause:0.94), (AMIODARONE HYDROCHLORIDE:Menopause:0.25), (AMISULPRIDE:Menopause:0.06), (AMLODIPINE BESYLATEHYDROCHLOROTHIAZIDEOLMESARTAN MEDOXOMIL:Menopause:0.33), (BENAZEPRILBENAZEPRIL HYDROCHLORIDE:Menopause:0.42), (BETAMETHASONECALCIPOTRIENE:Menopause: 0.19), (BICALUTAMIDE:Menopause:0.76), (BIFIDOBACTERIUM LONGUM SUBSP. INFANTIS:Menopause:0.41), (BISACODYL:Menopause:0.13), (BORTEZOMIB:Menopause:0.1 1), (BRINZOLAMIDE:Menopause:0.36), (BUPRENORPHINE HYDROCHLORIDE:Menopause:0.4), (CALCIPOTRIENE:Menopause:0.21), (CALCIUMMAG NESIUMZINC:Menopause:0.19), (CANAGLIFLOZINMETFORMIN HYDROCHLORIDE:Menopause:0.1), (CANAKINUMAB:Menopause:0.28), (CANDESARTAN:Menopause:0.7), (CANDESARTAN CILEXETILHYDROCHLOROTHIAZIDE:Menopause:0.54), (CARFILZOMIB:Menopause:0.2 8), (CETIRIZINE HYDROCHLORIDEPSEUDOEPHEDRINE HYDROCHLORIDE:Menopause:0.49), (CIMETIDINE:Menopause:0.63), (COLCHICINE:Meno pause:0.84), (CORTICOTROPIN:Menopause:0.38), (DEVICE:Menopause:0.51), (DIENOGEST: Menopause:0.06), (DIMENHYDRINATE:Menopause:0.69), (DIPYRONE:Menopause:0.14), (DR ONABINOL:Menopause:0.93), (DUTASTERIDE:Menopause:0.68), (ESOMEPRAZOLE MAGNESIUMNAPROXEN:Menopause:0.57), (ETHINYL ESTRADIOLNORGESTREL:Menopause:0.1), (ETORICOXIB:Menopause:0.48), (EVEROLIM US:Menopause:0.49), (FENTANYL CITRATE:Menopause:0.02), (FLUOCINONIDE:Menopause:0.5), (FLUTICASONE FUROATESALMETEROL XINAFOATE:Menopause:0.15), (FLUVOXAMINE MALEATE:Menopause:0.09), (FORMOTEROL FUMARATE:Menopause:0.47), (GLYCOPYRROLATE:Menopause:0.02), (HALOPERIDOL:M enopause:0.2), (HYDRALAZINE HYDROCHLORIDE:Menopause:0.5), (HYDROCHLOROTHIAZIDEIRBESARTAN:Menopau se:0.39), (HYDROCORTISONE ACETATE:Menopause:0.18), (IMIPRAMINE HYDROCHLORIDE:Menopause:0.8), (IPRATROPIUM:Menopause:0.73), (ISOPROPYL ALCOHOLLEUPROLIDE ACETATENORETHINDRONE ACETATE:Menopause:0.61), (ISOSORBIDE:Menopause:0.59), (ISOSORBIDE MONONITRATE:Menopause:0.99), (LABETALOLLABETALOL HYDROCHLORIDE:Menopause:0.41), (LEDIPASVIRSOFOSBUVIR:Menopause:0.22), (LID OCAINE HYDROCHLORIDE:Menopause:0.36), (LINAGLIPTIN:Menopause:0.44), (LITHIUM CARBONATE:Menopause:0.55), (LURASIDONE HYDROCHLORIDE:Menopause:0.06), (MEPOLIZUMAB:Menopause:0.35), (METHENAMIN E HIPPURATE:Menopause:0.34), (METHYLPREDNISOLONE ACETATE:Menopause:1.23), (MUPIROCINMUPIROCIN CALCIUM:Menopause:0.28), (NALOXONE HYDROCHLORIDEOXYCODONE HYDROCHLORIDE:Menopause:0.17), (NILOTINIB:Menopause:0.82), (OXYBUTYNINOXY BUTYNIN CHLORIDE:Menopause:0.08), (PAMIDRONATE DISODIUM:Menopause:0.33), (PAZOPANIB HYDROCHLORIDE:Menopause:0.49), (PEGFILGRASTIM:Menopause:0.21), (PEGINTERFER ON BETA-1A:Menopause:0.89), (PHENOBARBITAL:Menopause:0.34), (PIMECROLIMUS:Menopause:0. 07), (PONATINIB:Menopause:0.33), (PRAZOSIN HYDROCHLORIDE:Menopause:0.25), (PROTRIPTYLINE HYDROCHLORIDE:Menopause:0.18), (PSYLLIUM HUSK:Menopause:0.21), (RANOLAZINE:Menopause:1.17), (SALMETEROL XINAFOATE:Menopause:0.06), (SULFAMETHOXAZOLE:Menopause:0.09), (SULINDAC:Me nopause:0.16), (TERBUTALINE SULFATE:Menopause:0.48), (AMITRIPTYLINE HYDROCHLORIDE:Menorrhagia:0.01), (ATROPINE SULFATEDIPHENOXYLATE HYDROCHLORIDE:Menorrhagia:0.22), (BUPRENORPHINE HYDROCHLORIDE:Menorrhagia:0.01), (BUPRENORPHINE HYDROCHLORIDENALOXONE HYDROCHLORIDE:Menorrhagia:0.29), (CALCIUM CARBONATECHOLECALCIFEROL:Menorrhagia:1.03), (CALCIUM CARBONATEVITAMIN D:Menorrhagia:0.1), (CALCIUM CITRATE:Menorrhagia:0.35), (CARISOPRODOL:Menorrhagia:0.33), (CERTOLIZUMAB PEGOL:Menorrhagia:1.63), (CHOLESTYRAMINE:Menorrhagia:0.63), (CLOZAPINE:Menorrha gia:0.03), (COLCHICINE:Menorrhagia:0.07), (CYPROTERONE ACETATEETHINYL ESTRADIOL:Menorrhagia:0.2), (DACLIZUMAB:Menorrhagia:0.17), (DASATINIB:Menorrhagi a:0.03), (DIGOXIN:Menorrhagia:0.4), (ELETRIPTAN HYDROBROMIDE:Menorrhagia:0.94), (ENOXAPARIN SODIUM:Menorrhagia:0.09), (EVOLOCUMAB:Menorrhagia:0.42), (FINGOLIMOD HYDROCHLORIDE:Menorrhagia:0.24), (GLIMEPIRIDE:Menorrhagia:0.1), (HYDROXYCHL OROQUINE DIPHOSPHATE:Menorrhagia:0.18), (IBRUTINIB:Menorrhagia:0.79), (INSULIN HUMAN:Menorrhagia:0.44), (IRBESARTAN:Menorrhagia:0.01), (METHYLPREDNISOLONE ACETATE:Menorrhagia:0.8), (MOXIFLOXACIN HYDROCHLORIDE:Menorrhagia:0.12), (NILOTINIB:Menorrhagia:0.1), (NITROFURANTOIN:Menorrhagia:0.49), (NORTRIPTYLINE:Menorrhagia:0.32), (OSPEMIFENE:Menorrhagia:0.25), (OXYBUTYNIN CHLORIDE:Menorrhagia:0.02), (PANTOPRAZOLE MAGNESIUM:Menorrhagia:0.2), (PAROXETINEPAROXETINE HYDROCHLORIDE:Menorrhagia:0.25), (PENTOSAN POLYSULFATE SODIUM:Menorrhagia:0.31), (PHENOBARBITAL:Menorrhagia:0.47), (POLYETHYLENE GLYCOLS:Menorrhagia:0.25), (PRAVASTATIN:Menorrhagia:0.54), (PRIMIDONE:Menorrhagi a:0.31), (RALOXIFENE HYDROCHLORIDE:Menorrhagia:0.01), (ROPINIROLE HYDROCHLORIDE:Menorrhagia:0.07), (SECUKINUMAB:Menorrhagia:0.36), (SODIUM CHLORIDE:Menorrhagia:0.25), (SULFAMETHOXAZOLE:Menorrhagia:0.18), (TAMSULOSI N HYDROCHLORIDE:Menorrhagia:0.01), (TERBUTALINE SULFATE:Menorrhagia:0.04), (TIOTROPIUM BROMIDE MONOHYDRATE:Menorrhagia:0.49), (VERAPAMIL HYDROCHLORIDE:Menorrhagia:0.55), (VITAMIN B:Menorrhagia:0.39), (CHOLESTYRAMINE:Menstrual cycle management:0.05), (DEXLANSOPRAZOLE:Menstrual cycle management:0.61), (ENALAPRIL:Menstrual cycle management:0.36), (ENOXAPARIN:Menstrual cycle management:0.13), (ESZOPICLONE:Menstrual cycle management:0.04), (FERROUS SULFATE:Menstrual cycle management:0.12), (HYDROXYZINE PAMOATE:Menstrual cycle management:0.2), (INDOMETHACIN:Menstrual cycle management:0.12), (ISOPROPYL ALCOHOLLEUPROLIDE ACETATENORETHINDRONE ACETATE:Menstrual cycle management:0.6), (LACOSAMIDE:Menstrual cycle management:0.03), (LIDOCAINE:Menstrual cycle management:0.01), (PAROXETINE:Menstrual cycle management:0), (PENICILLIN:Menstrual cycle management:0.09), (PROBIOTICS NOS:Menstrual cycle management:0.13), (TERIFLUNOMIDE:Menstrual cycle management:0.03), (TIZANIDINE HYDROCHLORIDE:Menstrual cycle management:0.35), (TRAMADOL:Menstrual cycle management:0.56), (VARENICLINE TARTRATE:Menstrual cycle management:0.65), (ZONISAMIDE:Menstrual cycle management:0.11), (METRONIDAZOLE:Menstrual disorder:0.14), (NORETHINDRONE ACETATE:Menstrual disorder:0.25), (VARENICLINE TARTRATE:Menstrual disorder:0.24), (DEXLANSOPRAZOLE:Menstruation irregular:0.18), (INDOMETHACIN:Menstruation irregular:0.23), (ISOPROPYL ALCOHOLLEUPROLIDE ACETATENORETHINDRONE ACETATE:Menstruation irregular:0.57), (LISDEXAMFETAMINE DIMESYLATE:Menstruation irregular:0.18), (OLANZAPINE:Menstruation irregular:0.23), (PAROXETINE:Menstruation irregular:0.1), (TERIFLUNOMIDE:Menstruation irregular:0.17), (.ALPHA.1-PROTEINASE INHIBITOR HUMAN:Mental disorder:0.27), (ACARBOSE:Mental disorder:0.3), (ACENOCOUMAROL:Mental disorder:0.29), (ACETAMINOPHENCAFFEINEOPIUM:Mental disorder:0.12), (ACETAMINOPHENOXYCODONEOXYCODONE HYDROCHLORIDE:Mental disorder:0.33), (ACETAZOLAMIDE:Mental disorder:0.64), (ALEMTUZUMAB:Mental disorder:0.07), (AMANTADINE HYDROCHLORIDE:Mental disorder:0.94), (BUPRENORPHINENALOXONE:Mental disorder:0.14), (BUTYLSCOPOLAMINE BROMIDE:Mental disorder:0.02), (CANAGLIFLOZINMETFORMIN HYDROCHLORIDE:Mental disorder:0.18), (CANDESARTAN:Mental disorder:1.46), (CARBIDOPA:Mental disorder:0.02), (CARFILZOMIB:Mental disorder:0.16), (CARVEDILOL PHOSPHATE:Mental disorder:0.45), (CERTOLIZUMAB PEGOL:Mental disorder:1.44), (CHLORTHALIDONE:Mental disorder:0.5), (CICLESONIDE:Mental disorder:0.58), (CILASTATIN SODIUMIMIPENEM:Mental disorder:0.24), (CILOSTAZOL:Mental disorder:0.05), (CORTICOTROPIN:Mental disorder:0.24), (DABIGATRAN:Mental disorder:0.15), (DACLIZUMAB:Mental disorder:0.51), (DAPAGLIFLOZIN PROPANEDIOL:Mental disorder:0.35), (DARBEPOETIN ALFA:Mental disorder:0.15), (DASATINIB:Mental disorder:1.2), (DESVENLAFAXINE:Mental disorder:0.36), (DIMENHYDRINATE:Mental disorder:0.67), (DOCUSATE CALCIUM:Mental disorder:0.39), (DOFETILIDE:Mental disorder:0.45), (DRONABINOL:Mental disorder:0.25), (DULAGLUTIDE:Mental disorder:0.18), (ECONAZOLE:Mental disorder:0.02), (EDOXABAN TOSYLATE:Mental disorder:0.1), (ELETRIPTAN HYDROBROMIDE:Mental disorder:1.25), (EMPAGLIFLOZINLINAGLIPTIN:Mental disorder:0.23), (ENOXAPARIN:Mental disorder:1.21), (EPINEPHRINE:Mental disorder:0.55), (ERYTHROMYCIN:Mental disorder:0.19), (ERYTHROPOIETIN:Mental disorder:0.36), (ESTROGENS, CONJUGATEDMEDROXYPROGESTERONE ACETATE:Mental disorder:0.27), (ESTRONESUS SCROFA OVARY:Mental disorder:0.08), (EVEROLIMUS:Mental disorder:0.64), (EVOLOCUMAB:Mental disorder:1.54), (FEBUXOSTAT:Mental disorder:0.63), (FERROUS FUMARATE:Mental disorder:0.13), (FERROUS SULFATEFERROUS SULFATE, DRIED:Mental disorder:1.74), (FISH OIL\OMEGA-3 FATTY ACIDS:Mental disorder:0.07), (FLECAINIDE:Mental disorder:1.04), (FLUDROCORTISONE ACETATE:Mental disorder:0.43), (FORMOTEROL FUMARATE:Mental disorder:0.59), (FORMOTEROL FUMARATEMOMETASONE FUROATE:Mental disorder:0.39), (GUANFACINE:Mental disorder:1.94), (HYDROCHLOROTHIAZIDEIRBESARTAN:Mental disorder:0.6), (HYDROXYCHLOROQUINE SULFATE:Mental disorder:1.12), (HYOSCYAMINE:Mental disorder:0.19), (IMATINIB MESYLATE:Mental disorder:0.54), (IMIPRAMINE HYDROCHLORIDE:Mental disorder:0.9), (INDAPAMIDEPERINDOPRIL:Mental disorder:1.01), (INFLUENZA VIRUS VACCINE:Mental disorder:0.49), (INSULIN DEGLUDEC:Mental disorder:0.38), (INTERFERON:Mental disorder:0.27), (IPRATROPIUM:Mental disorder:0.84), (LEVOMEFOLATE CALCIUM:Mental disorder:0.2), (LITHIUM SULFATE:Mental disorder:0.41), (MEPOLIZUMAB:Mental disorder:0.25), (METHENAMINE HIPPURATE:Mental disorder:0.61), (MIDODRINE:Mental disorder:0.24), (MIDODRINE HYDROCHLORIDE:Mental disorder:0.12), (MINOCYCLINEMINOCYCLINE HYDROCHLORIDE:Mental disorder:0.11), (MOXIFLOXACIN:Mental disorder:0.02), (NABUMETONE:Mental disorder:0.3), (NORETHINDRONE:Mental disorder:0.45), (OXYMORPHONE:Mental disorder:0.17), (PENICILLIN:Mental disorder:0.67), (POSACONAZOLE:Mental disorder:0.06), (PRAZOSIN HYDROCHLORIDE:Mental disorder:0.14), (PROMAZINE HYDROCHLORIDE:Mental disorder:0.19), (PYRIDOXINE:Mental disorder:0.14), (RASAGILINE:Mental disorder:0.05), (RASAGILINE MESYLATE:Mental disorder:0.23), (RISEDRONATE SODIUM:Mental disorder:0.23), (RIVASTIGMINE TARTRATE:Mental disorder:0.98), (SCOPOLAMINE:Mental disorder:0.99), (SELEGILINE HYDROCHLORIDE:Mental disorder:0.39), (SUMATRIPTANSUMATRIPTAN SUCCINATE:Mental disorder:1.26), (TELMISARTAN:Mental disorder:1.44), (TETRACYCLINE:Mental disorder:0.82), (THALIDOMIDE:Mental disorder:0.19), (THEOPHYLLINE ANHYDROUS:Mental disorder:0.02), (TIOTROPIUM:Mental disorder:0.46), (TIZANIDINE HYDROCHLORIDE:Mental disorder:1.26), (TOLTERODINE TARTRATE:Mental disorder:0.55), (VINCRISTINE SULFATE:Mental disorder:0.18), (VITAMIN B:Mental disorder:1.1), (ZINC:Mental disorder:0.29), (ZONISAMIDE:Mental disorder:0.81), (METHYLPREDNISOLONE SODIUM SUCCINATE:Metabolic acidosis:0.11), (OXYGEN:Metabolic acidosis:0.04), (SPIRONOLACTONE:Metabolic acidosis:0.41), (CALCIUM CARBONATECHOLECALCIFEROL:Metabolic disorder:0), (DEXLANSOPRAZOLE:Metabolic disorder:0.07), (DILTIAZEM:Metabolic disorder:0.03), (DILTIAZEM HYDROCHLORIDE:Metabolic disorder:0.07), (EVEROLIMUS:Metabolic disorder:0.01), (EVOLOCUMAB:Metabolic disorder:0.38), (LENALIDOMIDE:Metabolic disorder:0.2), (MONTELUKAST SODIUM:Metabolic disorder:0.04), (NAPROXEN:Metabolic disorder:0.29), (PANTOPRAZOLE:Metabolic disorder:0.16), (RISEDRONATE SODIUM:Metabolic disorder:0), (TERIFLUNOMIDE:Metabolic disorder:0.75), (TOPIRAMATE:Metabolic disorder:0.16), (ACETAMINOPHENCAFFEINE:Metastases to bone:0.2), (ADENINE:Metastases to bone:0.4), (AGALSIDASE BETA:Metastases to bone:0.66), (ALBUTEROLIPRATROPIUM:Metastases to bone:0.14), (ALISKIREN HEMIFUMARATE:Metastases to bone:0.4), (AMANTADINE HYDROCHLORIDE:Metastases to bone:0.12), (AMBROXOLCLENBUTEROL:Metastases to bone:0.52), (AMEZINIUM METILSULFATE:Metastases to bone:0.21), (AMINO ACIDSDEXTROSEELECTROLYTES NOS:Metastases to bone:0.09), (AMISULPRIDE:Metastases to bone:0.74), (AMLODIPINE BESYLATEHYDROCHLOROTHIAZIDEVALSARTAN:Metastases to bone:0.5), (AMMONIUM LACTATE:Metastases to bone:0.12), (AMPHETAMINE ASPARTATEAMPHETAMINE SULFATEDEXTROAMPHETAMINE SACCHARATEDEXTROAMPHETAMINE SULFATE:Metastases to bone:0.84), (AMPICILLIN SODIUM:Metastases to bone:0.89), (ANAGRELIDE HYDROCHLORIDE:Metastases to bone:0.07),(ARMODAFINIL:Metastases to bone:0.58), (ASPIRINCLOPIDOGREL:Metastases to bone:0.01), (ASPIRINDIHYDROXYALUMINUM AMINOACETATE ANHYDROUSMAGNESIUM CARBONATE:Metastases to bone:0.13), (ATROPINE SULFATE:Metastases to bone:0.27), (ATROPINEDIPHENOXYLATE:Metastases to bone:0.09), (AVELUMAB:Metastases to bone:0.01), (AZACITIDINE:Metastases to bone:0.51), (BENDAMUSTINE:Metastases to bone:0.13), (BENZTROPINE:Metastases to bone:0.02), (BEPOTASTINE:Metastases to bone:0.21), (BETAHISTINE MESILATE:Metastases to bone:0.52), (BETAMETHASONECALCIPOTRIENE:Metastases to bone:0.02), (BILASTINE:Metastases to bone:0.35), (BOSUTINIB:Metastases to bone:0.06), (BUDESONIDEFORMOTEROL FUMARATE:Metastases to bone:0.46), (BUPRENORPHINE HYDROCHLORIDENALOXONE HYDROCHLORIDE:Metastases to bone:0.15), (CALCITONIN SALMON:Metastases to bone:1.29), (CALCIUM ACETATE:Metastases to bone:0.91), (CALCIUM CARBONATEERGOCALCIFEROLRETINOL:Metastases to bone:0.15), (CALCIUMMAGNESIUM:Metastases to bone:0.26), (CANAGLIFLOZINMETFORMIN HYDROCHLORIDE:Metastases to bone:0.18), (CANAKINUMAB:Metastases to bone:0.59), (CARBOHYDRATESPOTASSIUM CHLORIDESODIUM CHLORIDESODIUM LACTATE:Metastases to bone:0.27), (CARBOMER:Metastases to bone:0.01), (CARTEOLOL HYDROCHLORIDE:Metastases to bone:0.23), (CARVEDILOL PHOSPHATE:Metastases to bone:1.46), (CEFPODOXIME PROXETIL:Metastases to bone:0.34), (CERTOLIZUMAB PEGOL:Metastases to bone:0.4), (CHIMAPHILA UMBELLATAMANGANESE CHLORIDEPOPULUS TREMULOIDESSODIUM TAUROCHOLATE:Metastases to bone:0.92), (CHLORAMBUCIL:Metastases to bone:0.35), (CHLORPHENIRAMINEHYDROCODONE:Metastases to bone:0.14), (CHLORPROPAMIDE:Metastases to bone:0.1), (CHONDROITIN SULFATE (BOVINE):Metastases to bone:0.22), (CICLESONIDE:Metastases to bone:0.11), (CICLOPIROX:Metastases to bone:0.04), (CIPROFIBRATE:Metastases to bone:0.56), (CLOZAPINE:Metastases to bone:1.09), (CODEINE PHOSPHATEGUAIFENESIN:Metastases to bone:0.07), (CORTICOTROPIN:Metastases to bone:0.14), (DACLIZUMAB:Metastases to bone:0.02), (DANAZOL:Metastases to bone:0.16), (DAPAGLIFLOZIN PROPANEDIOL:Metastases to bone:0.05), (DEFLAZACORT:Metastases to bone:1.03), (DESMOPRESSIN:Metastases to bone:0.36), (DILAZEP:Metastases to bone:0.24), (DOFETILIDE:Metastases to bone:0.47), (DORZOLAMIDE:Metastases to bone:0.67), (DOXEPIN HYDROCHLORIDE:Metastases to bone:0.2), (DRONEDARONE:Metastases to bone:0.29), (EDOXABAN TOSYLATE:Metastases to bone:1.8), (ELCATONIN:Metastases to bone:0.26), (ELETRIPTAN HYDROBROMIDE:Metastases to bone:0.96), (ELOTUZUMAB:Metastases to bone:0.76), (EPACADOSTAT:Metastases to bone:0.27), (EPINEPHRINE:Metastases to bone:0.56), (ESTROGENS:Metastases to bone:0.25), (ESTRONESUS SCROFA OVARY:Metastases to bone:0.24), (ETRETINATE:Metastases to bone:1.23), (EVOLOCUMAB:Metastases to bone:0.12), (FENOFIBRIC ACID:Metastases to bone:0.38), (FEXOFENADINEPSEUDOEPHEDRINE:Metastases to bone:0.4), (FLUDROCORTISONE ACETATE:Metastases to bone:1.15), (FLUTICASONE FUROATE:Metastases to bone:0.54), (FONDAPARINUX:Metastases to bone:0.22), (FORMOTEROL FUMARATE DIHYDRATEMOMETASONE FUROATE:Metastases to bone:0.21), (FOSAPREPITANT DIMEGLUMINE:Metastases to bone:0.1), (FUSIDIC ACID:Metastases to bone:0.75), (HERBALSSAW PALMETTO:Metastases to bone:0.47), (HUMAN PLATELET, ALLOGENIC:Metastases to bone:0.04), (HYDROCHLOROTHIAZIDEQUINAPRIL HYDROCHLORIDE:Metastases to bone:0.31), (ICOSAPENT:Metastases to bone:0. 11), (IDELALISIB:Metastases to bone:0.38), (IGURATIMOD:Metastases to bone:0.32), (ILOPROST:Metastases to bone:0.44), (IMIPRAMINE HYDROCHLORIDE:Metastases to bone:0.23), (INDAPAMIDEPERINDOPRIL:Metastases to bone:2.21), (INTERFERON ALFA-2A:Metastases to bone:0.87), (INTERFERON ALFA-2B:Metastases to bone:1.21), (IODINE:Metastases to bone:0.3), (IRONPOLYSACCHARIDES:Metastases to bone:0.12), (LACOSAMIDE:Metastases to bone:0.43), (LACTOBACILLUS ACIDOPHILUSLACTOBACILLUS DELBRUECKII SUBSP. BULGARICUS:Metastases to bone:0.07), (LANREOTIDE:Metastases to bone:0.76), (LEDIPASVIRSOFOSBUVIR:Metastases to bone:0.96), (LEVOCABASTINE:Metastases to bone:0.06), (LEVOCABASTINE HYDROCHLORIDE:Metastases to bone:0.14), (LINSIDOMINE:Metastases to bone:0.17), (LISDEXAMFETAMINE DIMESYLATE:Metastases to bone:0.01), (LITHIUM CARBONATE:Metastases to bone:1.45), (LUTEIN:Metastases to bone:0.55), (MAGALDRATE:Metastases to bone:0.04), (MAGNESIUM PIDOLATE:Metastases to bone:0), (MEPERIDINE:Metastases to bone:0.51), (METFORMIN HYDROCHLORIDEVILDAGLIPTIN:Metastases to bone:1.11), (METFORMIN PAMOATE:Metastases to bone:0.08), (METHENAMINE HIPPURATE:Metastases to bone:0.9), (MIDODRINE HYDROCHLORIDE:Metastases to bone:0), (MIRIPLATIN:Metastases to bone:0.01), (MOSAPRIDE CITRATE DIHYDRATE:Metastases to bone:0.21), (NICARDIPINE:Metastases to bone:0.76), (NICARDIPINE HYDROCHLORIDE:Metastases to bone:0.39), (NILOTINIB:Metastases to bone:2.36), (OXCARBAZEPINE:Metastases to bone:1.79), (PANTOPRAZOLE MAGNESIUM:Metastases to bone:1.16), (PEGINTERFERON ALFA-2B:Metastases to bone:0.06), (PEGINTERFERON BETA-1A:Metastases to bone:0.17), (PENICILLIN G:Metastases to bone:0.22), (PENTOSAN POLYSULFATE SODIUM:Metastases to bone:0.22), (PIRFENIDONE:Metastases to bone:0.03), (PIRITRAMIDE:Metastases to bone:0.07), (PONATINIB:Metastases to bone:0.53), (POTASSIUM CITRATETRISODIUM CITRATE DIHYDRATE:Metastases to bone:0.53), (PRAMIPEXOLE DIHYDROCHLORIDE:Metastases to bone:1.17), (PRANLUKAST:Metastases to bone:0.87), (PRAZOSIN:Metastases to bone:0.15), (PRAZOSIN HYDROCHLORIDE:Metastases to bone:0.04), (PRIMIDONE:Metastases to bone:1.39), (PRISTINAMYCIN:Metastases to bone:0.01), (PROGESTERONE:Metastases to bone:0.83), (PROPAFENONE HYDROCHLORIDE:Metastases to bone:0.22), (RALOXIFENE HYDROCHLORIDE:Metastases to bone:0.61), (RALTITREXED:Metastases to bone:0.03), (RETINOL:Metastases to bone:0.22), (RIVASTIGMINE TARTRATE:Metastases to bone:0.36), (RIZATRIPTAN BENZOATE:Metastases to bone:0.39), (ROFLUMILAST:Metastases to bone:0.97), (RUCAPARIB CAMSYLATE:Metastases to bone:0.22), (SALMETEROL XINAFOATE:Metastases to bone:0.05), (SARPOGRELATE:Metastases to bone:1.31), (SEVELAMER CARBONATE:Metastases to bone:0.12), (SEVELAMER HYDROCHLORIDE:Metastases to bone:0.68), (SODIUM OXYBATE:Metastases to bone:0.35), (STARCH, WHEAT:Metastases to bone:0.12), (SULFAMETHOXAZOLE:Metastases to bone:0.8), (SULINDAC:Metastases to bone:0.02), (SUMATRIPTANSUMATRIPTAN SUCCINATE:Metastases to bone:0.44), (SUPLATAST TOSILATE:Metastases to bone:0.05), (TAFLUPROST:Metastases to bone:0.02), (TECELEUKIN:Metastases to bone:0.16), (TERBINAFINE HYDROCHLORIDE:Metastases to bone:0.76), (TERBUTALINE:Metastases to bone:0.19), (TERIFLUNOMIDE:Metastases to bone:0.23), (TESTOSTERONE UNDECANOATE:Metastases to bone:0.54), (TETRACYCLINE:Metastases to bone:0.59), (TILIDINE:Metastases to bone:0.12), (TIOTROPIUM BROMIDE:Metastases to bone:0.08), (TRABECTEDIN:Metastases to bone:0.37), (UMECLIDINIUM BROMIDEVILANTEROL TRIFENATATE:Metastases to bone:0.4), (VALGANCICLOVIR HYDROCHLORIDE:Metastases to bone:0.58), (VARENICLINE TARTRATE:Metastases to bone:0.58), (VINCRISTINE SULFATE:Metastases to bone:0.35), (VITAMIN A:Metastases to bone:0.42), (WARFARIN POTASSIUM:Metastases to bone:0.94), (XIPAMIDE:Metastases to bone:0.13), (ZIV-AFLIBERCEPT:Metastases to bone:0.87), (ZOLMITRIPTAN:Metastases to bone:0.13), (GOSERELIN ACETATE:Metastases to bone marrow:0.01), (ALCOHOL:Metastases to central nervous system:0.14), (ALENDRONIC ACID:Metastases to central nervous system:0.01), (AMIODARONE:Metastases to central nervous system:0.55), (AMIODARONE HYDROCHLORIDE:Metastases to central nervous system:0.23), (BISACODYL OR DOCUSATE SODIUM:Metastases to central nervous system:0.01), (BUDESONIDE:Metastases to central nervous system:0.6), (BUPROPION:Metastases to central nervous system:0.06), (CALCITRIOL:Metastases to central nervous system:0.04), (CALCIUMVITAMIN D:Metastases to central nervous system:0.15), (CANDESARTAN:Metastases to central nervous system:0.87), (CEFTRIAXONE SODIUM:Metastases to central nervous system:0.01), (CELECOXIB:Metastases to central nervous system:0.55), (CERTOLIZUMAB PEGOL:Metastases to central nervous system:0.45), (CLOBAZAM:Metastases to central nervous system:0.1 1), (CLONIDINE:Metastases to central nervous system:0.23), (DASATINIB:Metastases to central nervous system:0.85), (DIGOXIN:Metastases to central nervous system:0.36), (DIVALPROEX SODIUM:Metastases to central nervous system:0.44), (DUTASTERIDE:Metastases to central nervous system:0.4), (ENALAPRIL MALEATE:Metastases to central nervous system:0.02), (EPINEPHRINE:Metastases to central nervous system:0.09), (ERIBULIN:Metastases to central nervous system:0.24), (ESZOPICLONE:Metastases to central nervous system:0.03), (FERROUS SULFATEFERROUS SULFATE, DRIED:Metastases to central nervous system:0.57), (IBRUTINIB:Metastases to central nervous system:1.14), (IDELALISIB:Metastases to central nervous system:0.35), (IMATINIB MESYLATE:Metastases to central nervous system:0.31), (INDAPAMIDEPERINDOPRIL:Metastases to central nervous system:0.12), (IRON:Metastases to central nervous system:0.18), (LEDIPASVIRSOFOSBUVIR:Metastases to central nervous system:0.03), (LENALIDOMIDE:Metastases to central nervous system:1.23), (LEVOLEUCOVORIN CALCIUM:Metastases to central nervous system:0.56), (MELOXICAM:Metastases to central nervous system:0.21), (MOXIFLOXACIN HYDROCHLORIDE:Metastases to central nervous system:0.39), (NILOTINIB:Metastases to central nervous system:0.89), (NITROFURANTOIN:Metastases to central nervous system:0.15), (NITROGLYCERIN:Metastases to central nervous system:0.04), (PAMIDRONATE DISODIUM:Metastases to central nervous system:0.17), (PITAVASTATIN CALCIUM:Metastases to central nervous system:0.09), (PRAVASTATIN:Metastases to central nervous system:0.28), (PRIMIDONE:Metastases to central nervous system:0.12), (QUETIAPINE:Metastases to central nervous system:0.13), (RISEDRONATE SODIUM:Metastases to central nervous system:0.09), (SOTALOL:Metastases to central nervous system:0.01), (THALIDOMIDE:Metastases to central nervous system:1.07), (WARFARIN SODIUM:Metastases to central nervous system:0.09), (ZIV-AFLIBERCEPT:Metastases to central nervous system:0), (ABIRATERONE:Metastases to liver:0.41), (ACETAMINOPHENBUTALBITALCAFFEINE:Metastases to liver:0.08), (AMIODARONE:Metastases to liver:1.11), (AMPHETAMINE ASPARTATEAMPHETAMINE SULFATEDEXTROAMPHETAMINE SACCHARATEDEXTROAMPHETAMINE SULFATE:Metastases to liver:0.05), (BUDESONIDE:Metastases to liver:0.81), (CALCITONIN SALMON:Metastases to liver:0.04), (CALCIUM CARBONATECHOLECALCIFEROL:Metastases to liver:1), (CARFILZOMIB:Metastases to liver:0.05), (CEFTRIAXONE SODIUM:Metastases to liver:0), (CERTOLIZUMAB PEGOL:Metastases to liver:0.67), (CHOLESTYRAMINE:Metastases to liver:1.04), (CORTISONEHYDROCORTISONE:Metastases to liver:0.63), (CYCLOBENZAPRINE:Metastases to liver:0.05), (CYCLOPHOSPHAMIDEEPIRUBICIN HYDROCHLORIDEFLUOROURACIL:Metastases to liver:0.46), (DABRAFENIB:Metastases to liver:0.97), (DALTEPARIN SODIUM:Metastases to liver:0.62), (DASATINIB:Metastases to liver:1.39), (DOXAZOSIN MESYLATE:Metastases to liver:0.39), (ENALAPRIL MALEATE:Metastases to liver:0.72), (ESZOPICLONE:Metastases to liver:0.38), (EVOLOCUMAB:Metastases to liver:0.16), (FLECAINIDE:Metastases to liver:0.05), (IBRUTINIB:Metastases to liver:1.54), (INDAPAMIDEPERINDOPRIL:Metastases to liver:0.45), (INTERFERON ALFA-2B:Metastases to liver:0.22), (ISOSORBIDE:Metastases to liver:0.09), (LEDIPASVIRSOFOSBUVIR:Metastases to liver:0.34), (LERCANIDIPINE HYDROCHLORIDE:Metastases to liver:0.15), (LINAGLIPTIN:Metastases to liver:0.55), (METFORMIN HYDROCHLORIDEVILDAGLIPTIN:Metastases to liver:0.1), (NADROPARIN CALCIUM:Metastases to liver:0.01), (NIACIN:Metastases to liver:0.06), (NITROFURANTOIN:Metastases to liver:0.73), (OMEGA-3-ACID ETHYL ESTERS:Metastases to liver:0.03), (OSIMERTINIB:Metastases to liver:0.26), (OXCARBAZEPINE:Metastases to liver:0.09), (PANTOPRAZOLE MAGNESIUM:Metastases to liver:0.33), (PEGINTERFERON ALFA-2B:Metastases to liver:0.09), (PONATINIB:Metastases to liver:0.1), (PRIMIDONE:Metastases to liver:0.2), (PROBIOTICS NOS:Metastases to liver:0.67), (RADIATION THERAPY:Metastases to liver:1.11), (RANOLAZINE:Metastases to liver:0.12), (RISEDRONATE SODIUM:Metastases to liver:0.37), (ROFLUMILAST:Metastases to liver:0.06), (SACUBITRILVALSARTAN:Metastases to liver:0.21), (SAMARIUM SM-153 LEXIDRONAM PENTASODIUM:Metastases to liver:0.3), (SCOPOLAMINE:Metastases to liver:0.1), (SIPULEUCEL-T:Metastases to liver:0.03), (SODIUM BICARBONATE:Metastases to liver:0.08), (TEGAFUR:Metastases to liver:0.06), (TINZAPARIN:Metastases to liver:0.04), (TOCILIZUMAB:Metastases to liver:0.14), (TOFACITINIB CITRATE:Metastases to liver:0.06), (TRAMETINIB:Metastases to liver:0.99), (VITAMIN B:Metastases to liver:0.16), (ABIRATERONE:Metastases to lung:0.14), (ACETAMINOPHENBUTALBITALCAFFEINE:Metastases to lung:0.13), (AMITRIPTYLINE HYDROCHLORIDE:Metastases to lung:0.02), (AMPHETAMINE ASPARTATEAMPHETAMINE SULFATEDEXTROAMPHETAMINE SACCHARATEDEXTROAMPHETAMINE SULFATE:Metastases to lung:0.1), (BROMAZEPAM:Metastases to lung:0.33), (BUSPIRONE HYDROCHLORIDE:Metastases to lung:0.09), (CALCITONIN SALMON:Metastases to lung:0.22), (CANDESARTAN CILEXETILHYDROCHLOROTHIAZIDE:Metastases to lung:0.82), (CARFILZOMIB:Metastases to lung:0.47), (CERTOLIZUMAB PEGOL:Metastases to lung:0.6), (CHOLESTYRAMINE:Metastases to lung:0.53), (CLOZAPINE:Metastases to lung:0.15), (CYCLOPHOSPHAMIDEEPIRUBICIN HYDROCHLORIDEFLUOROURACIL:Metastases to lung:0.11), (DASATINIB:Metastases to lung:1.36), (DIMENHYDRINATE:Metastases to lung:0.06), (DUTASTERIDE:Metastases to lung:0.75), (EDOXABAN TOSYLATE:Metastases to lung:0.06), (EPINEPHRINE:Metastases to lung:0.62), (EVOLOCUMAB:Metastases to lung:0.38), (EZETIMIBESIMVASTATIN:Metastases to lung:0.18), (FLECAINIDE:Metastases to lung:0.39), (FLUOXETINEFLUOXETINE HYDROCHLORIDE:Metastases to lung:0.11), (GLYBURIDE:Metastases to lung:0.07), (IBRUTINIB:Metastases to lung:1.53), (ILOPROST:Metastases to lung:0.12), (INDAPAMIDEPERINDOPRIL:Metastases to lung:0.46), (INFLUENZA VIRUS VACCINE:Metastases to lung:0.01), (INTERFERON ALFA-2B:Metastases to lung:0.2), (INVESTIGATIONAL PRODUCT:Metastases to lung:0.14), (ISOSORBIDE:Metastases to lung:0.38), (LABETALOLLABETALOL HYDROCHLORIDE:Metastases to lung:0.15), (LEDIPASVIRSOFOSBUVIR:Metastases to lung:0.57), (LENALIDOMIDE:Metastases to lung:0.9), (LERCANIDIPINE HYDROCHLORIDE:Metastases to lung:0.32), (METAXALONE:Metastases to lung:0.01), (MINOCYCLINEMINOCYCLINE HYDROCHLORIDE:Metastases to lung:0.11), (MOXIFLOXACIN HYDROCHLORIDE:Metastases to lung:0.63), (NEBIVOLOL HYDROCHLORIDE:Metastases to lung:0.3), (NILOTINIB:Metastases to lung:1.63), (NIRAPARIB:Metastases to lung:0.44), (OLAPARIB:Metastases to lung:0.88), (OMEGA-3-ACID ETHYL ESTERS:Metastases to lung:0.29), (OXCARBAZEPINE:Metastases to lung:0.13), (PITAVASTATIN CALCIUM:Metastases to lung:0.39), (PONATINIB:Metastases to lung:0.13), (PRAMIPEXOLE DIHYDROCHLORIDE:Metastases to lung:0.26), (PRIMIDONE:Metastases to lung:0.36), (QUETIAPINE:Metastases to lung:0.23), (RABEPRAZOLE:Metastases to lung:0.22), (RANOLAZINE:Metastases to lung:0.58), (SCOPOLAMINE:Metastases to lung:0.09), (SIPULEUCEL-T:Metastases to lung:0), (SULFAMETHOXAZOLE:Metastases to lung:0.01), (THALIDOMIDE:Metastases to lung:0.56), (TINZAPARIN:Metastases to lung:0.22), (TIOTROPIUM:Metastases to lung:0.01), (TIZANIDINE HYDROCHLORIDE:Metastases to lung:0.04), (TOFACITINIB CITRATE:Metastases to lung:0.08), (TOPIRAMATE:Metastases to lung:0.9), (UMECLIDINIUM BROMIDEVILANTEROL TRIFENATATE:Metastases to lung:0.16), (VALACYCLOVIR HYDROCHLORIDE:Metastases to lung:0.26), (VITAMIN B:Metastases to lung:0.32), (CYCLOPHOSPHAMIDEEPIRUBICIN HYDROCHLORIDEFLUOROURACIL:Metastases to lymph nodes:0.04), (DABRAFENIB:Metastases to lymph nodes:0.26), (DEXLANSOPRAZOLE:Metastases to lymph nodes:0.3), (IBRUTINIB:Metastases to lymph nodes:0.18), (IMATINIB MESYLATE:Metastases to lymph nodes:0.05), (LOVASTATIN:Metastases to lymph nodes:0.22), (MONTELUKAST SODIUM:Metastases to lymph nodes:0.01), (NILOTINIB:Metastases to lymph nodes:0.43), (OLAPARIB:Metastases to lymph nodes:0.35), (PANTOPRAZOLE:Metastases to lymph nodes:0.43), (RADIATION THERAPY:Metastases to lymph nodes:0.38), (TAMSULOSIN:Metastases to lymph nodes:0.16), (THALIDOMIDE:Metastases to lymph nodes:0.35), (TRAMETINIB:Metastases to lymph nodes:0.28), (ZIV-AFLIBERCEPT:Metastases to lymph nodes:0.01), (LEVOLEUCOVORIN CALCIUM:Metastases to peritoneum:0.06), (ALBUTEROL SULFATE:Metastasis:0.42), (ALBUTEROL SULFATEIPRATROPIUM BROMIDE:Metastasis:0.07), (ALENDRONIC ACID:Metastasis:0.18), (BISACODYL OR DOCUSATE SODIUM:Metastasis:0.13), (BUPROPION HYDROCHLORIDE:Metastasis:0.29), (CANDESARTAN CILEXETIL:Metastasis:0.5), (CERTOLIZUMAB PEGOL:Metastasis:0.21), (CIMETIDINE:Metastasis:0.2), (COLCHICINE:Metastasis:0.25), (CY CLOPHOSPHAMIDEEPIRUBICIN HYDROCHLORIDEFLUOROURACIL:Metastasis:0.23), (DALTEPARIN SODIUM:Metastasis:0.63), (DASATINIB:Metastasis:1.13), (DEXLANSOPRAZOLE:Metastasis: 0.64), (DIGOXIN:Metastasis:0.73), (DILTIAZEM HYDROCHLORIDE:Metastasis:0.54), (DRONABINOL:Metastasis:0.33), (ESZOPICLONE:Met astasis:0.14), (FERROUS SULFATEFERROUS SULFATE, DRIED:Metastasis:0.63), (FLUTICASONE PROPIONATE:Metastasis:0.53), (GLIPIZIDE:Metastasis:0.12), (INSULIN HUMAN:Metastasis:0.04), (INTERFERON ALFA-2B:Metastasis:0.31), (LEDIPASVIRSOFOSBUVIR:Metastasis:0.09), (LERCANIDIPINE HYDROCHLORIDE:Metastasis:0.05), (LIDOCAINE:Metastasis:0.36), (LINAGLIPTIN:Metasta sis:0.17), (MAGNESIUM:Metastasis:0.57), (MELOXICAM:Metastasis:0.25), (METRONIDAZO LEMETRONIDAZOLE HYDROCHLORIDE:Metastasis:0.01), (NEBIVOLOL:Metastasis:0.09), (NILOTINIB:Metastasis:1.02), (OLAPARIB:Metastasis:0.7), (OXCARBAZEPINE:Metastasis:0.21), (PITAVASTATIN CALCIUM:Metastasis:0.15), (PRIMIDONE:Metastasis:0.07), (RADIATION THERAPY:Metastasis:0.86), (RANOLAZINE:Metastasis:0.01), (RISEDRONATE SODIUM:Metastasis:0.06), (SACUBITRILVALSARTAN:Metastasis:0.07), (SENNOSIDES:Me tastasis:0.09), (TINZAPARIN SODIUM:Metastasis:0.68), (TOPIRAMATE:Metastasis:0.44), (VERAPAMIL HYDROCHLORIDE:Metastasis:0.34), (ZIV-AFLIBERCEPT:Metastasis:0.15), (ABIRATERONE ACETATE:Metastatic malignant melanoma:0.1 1), (ACETAMINOPHENOXYCODONEOXYCODONE HYDROCHLORIDE:Metastatic malignant melanoma:0.72), (ACETAMINOPHENPROPOXYPHENE HYDROCHLORIDE:Metastatic malignant melanoma:0.35), (ACETAMINOPHENPROPOXYPHENE NAPSYLATE:Metastatic malignant melanoma:0.07), (ADO-TRASTUZUMAB EMTANSINE:Metastatic malignant melanoma:0.69), (AGALSIDASE BETA:Metastatic malignant melanoma:0.53), (ALCOHOL:Metastatic malignant melanoma:0.2), (ALISKIREN HEMIFUMARATE:Metastatic malignant melanoma:0.3), (AMANTADINE:Metastatic malignant melanoma:0.33), (AMANTADINE HYDROCHLORIDE:Metastatic malignant melanoma:0.27), (AMIKACIN SULFATE:Metastatic malignant melanoma:0.14), (AMLODIPINE BESYLATEBENAZEPRIL HYDROCHLORIDE:Metastatic malignant melanoma:0), (AMLODIPINE BESYLATEHYDROCHLOROTHIAZIDEVALSARTAN:Metastatic malignant melanoma:0.65), (AMPICILLIN SODIUMSULBACTAM SODIUM:Metastatic malignant melanoma:0.04), (ANASTROZOLE:Metastatic malignant melanoma:1.81), (ANTIHISTAMINES NOS:Metastatic malignant melanoma:0.12), (ASPIRINCLOPIDOGREL:Metastatic malignant melanoma:0.2), (ATROPINE SULFATE:Metastatic malignant melanoma:0.16), (AXITINIB:Metastatic malignant melanoma:0.51), (AZELASTINE HYDROCHLORIDE:Metastatic malignant melanoma:0. 02), (BENAZEPRILBENAZEPRIL HYDROCHLORIDE:Metastatic malignant melanoma:0.85), (BETAMETHASONE DIPROPIONATECALCIPOTRIENE:Metastatic malignant melanoma:0.07), (BIFIDOBACTERIUM LONGUM SUBSP. INFANTIS:Metastatic malignant melanoma:0.06), (BILASTINE:Metastatic malignant melanoma:0.61), (BUDESONIDEFORMOTEROL:Metastatic malignant melanoma:0.04), (BUDESONIDEFORMOTEROL FUMARATE:Metastatic malignant melanoma:0.39), (BUPRENORPHINE HYDROCHLORIDENALOXONE HYDROCHLORIDE:Metastatic malignant melanoma:0.11), (CALCITONIN SALMON:Metastatic malignant melanoma:0.47), (CALCIUM ACETATE:Metastatic malignant melanoma:1.38), (CALCIUMMAGNESIUM:Metastatic malignant melanoma:0.09), (CANAGLIFLOZINMETFORMIN HYDROCHLORIDE:Metastatic malignant melanoma:0.58), (CANAKINUMAB:Metastatic malignant melanoma:0.07), (CARFILZOMIB:Metastatic malignant melanoma:1.13), (CARVEDILOL PHOSPHATE:Metastatic malignant melanoma:1.16), (CASANTHRANOLDOCUSATE SODIUM:Metastatic malignant melanoma:0), (CEFEPIMECEFEPIME HYDROCHLORIDE:Metastatic malignant melanoma:0.15), (CEFTAZIDIME:Metastatic malignant melanoma:0.65), (CERITINIB:Metastatic malignant melanoma:0.34), (CERTOLIZUMAB PEGOL:Metastatic malignant melanoma:1.07), (CHLORAMBUCIL:Metastatic malignant melanoma:0.29), (CHLORDIAZEPOXIDE HYDROCHLORIDECLIDINIUM BROMIDE:Metastatic malignant melanoma:0.28), (CHLORHEXIDINE GLUCONATE:Metastatic malignant melanoma:0.08), (CHONDROITIN SULFATE (BOVINE)GLUCOSAMINE HYDROCHLORIDE:Metastatic malignant melanoma:0.4), (CIPROFIBRATE:Metastatic malignant melanoma:0.24), (COLESEVELAM HYDROCHLORIDE:Metastatic malignant melanoma:0.39), (CORTICOTROPIN:Metastatic malignant melanoma:0.04), (CYAMEMAZINE:Metastatic malignant melanoma:0.24), (DACLIZUMAB:Metastatic malignant melanoma:1.23), (DAPAGLIFLOZIN PROPANEDIOL:Metastatic malignant melanoma:0.2), (DAPSONE:Metastatic malignant melanoma:0.19), (DEFLAZACORT:Metastatic malignant melanoma:0.28), (DICYCLOMINE HYDROCHLORIDE:Metastatic malignant melanoma:1.18), (DOFETILIDE:Metastatic malignant melanoma:0.79), (DOLASETRON MESYLATE:Metastatic malignant melanoma:0.31), (DUTASTERIDETAMSULOSIN HYDROCHLORIDE:Metastatic malignant melanoma:0.06), (EDOXABAN TOSYLATE:Metastatic malignant melanoma:0.89), (ELETRIPTAN HYDROBROMIDE:Metastatic malignant melanoma:1.04), (EMPAGLIFLOZINLINAGLIPTIN:Metastatic malignant melanoma:0.29), (EVOLOCUMAB:Metastatic malignant melanoma:0.74), (FENOTEROL HYDROBROMIDEIPRATROPIUM BROMIDE:Metastatic malignant melanoma:0.22), (FEXOFENADINEPSEUDOEPHEDRINE:Metastatic malignant melanoma:0.11), (FINGOLIMOD HYDROCHLORIDE:Metastatic malignant melanoma:0.38), (FLUTICASONE FUROATEVILANTEROL TRIFENATATE:Metastatic malignant melanoma:0.93), (FLUVOXAMINE MALEATE:Metastatic malignant melanoma:0.25), (FORMOTEROL FUMARATE:Metastatic malignant melanoma:0.78), (GEMCITABINE HYDROCHLORIDE:Metastatic malignant melanoma:0.32), (HEPARIN CALCIUM:Metastatic malignant melanoma:1. 11), (HERBALS:Metastatic malignant melanoma:0.16), (HERBALSTURMERIC:Metastatic malignant melanoma:0.46), (HYDROXYCHLOROQUINE DIPHOSPHATE:Metastatic malignant melanoma:0.19), (IDELALISIB:Metastatic malignant melanoma:0.26), (ILOPROST:Metastatic malignant melanoma:0.16), (IMATINIB MESYLATE:Metastatic malignant melanoma:0.91), (IMIPENEM:Metastatic malignant melanoma:0.33), (IMIPRAMINE HYDROCHLORIDE:Metastatic malignant melanoma:0.41), (INFLIXIMAB-DYYB:Metastatic malignant melanoma:0.72), (IRINOTECAN HYDROCHLORIDE:Metastatic malignant melanoma:0.32), (ISOSORBIDE:Metastatic malignant melanoma:1.06), (ISOSORBIDE DINITRATE:Metastatic malignant melanoma:0.15), (LAPATINIB DITOSYLATE:Metastatic malignant melanoma:0.13), (LEDIPASVIRSOFOSBUVIR:Metastatic malignant melanoma:0.62), (LENVATINIB:Metastatic malignant melanoma:0.05), (LEUCOVORIN CALCIUM:Metastatic malignant melanoma:0.29), (LEVALBUTEROL HYDROCHLORIDE:Metastatic malignant melanoma:1.26), (LINACLOTIDE:Metastatic malignant melanoma:0.02), (LIOTHYRONINE:Metastatic malignant melanoma:0.01), (LISDEXAMFETAMINE DIMESYLATE:Metastatic malignant melanoma:0), (LITHIUM CARBONATE:Metastatic malignant melanoma:1.14), (LOMITAPIDE MESYLATE:Metastatic malignant melanoma:0.08), (MELPERONE:Metastatic malignant melanoma:0.15), (METAXALONE:Metastatic malignant melanoma:1.15), (METFORMIN HYDROCHLORIDEVILDAGLIPTIN:Metastatic malignant melanoma:0.51), (METHENAMINE HIPPURATE:Metastatic malignant melanoma:0.94), (METHYLPREDNISOLONE ACETATE:Metastatic malignant melanoma:1.34), (MIDODRINE HYDROCHLORIDE:Metastatic malignant melanoma:0.06), (MINOCYCLINE HYDROCHLORIDE:Metastatic malignant melanoma:0.6), (MORPHINE SULFATENALTREXONE HYDROCHLORIDE:Metastatic malignant melanoma:0.18), (MUPIROCINMUPIROCIN CALCIUM:Metastatic malignant melanoma:0.61), (NILOTINIB:Metastatic malignant melanoma:1.21), (NINTEDANIB:Metastatic malignant melanoma:0.37), (NIRAPARIB:Metastatic malignant melanoma:0.2), (NOREPINEPHRINE:Metastatic malignant melanoma:0.59), (NOREPINEPHRINE BITARTRATE:Metastatic malignant melanoma:0.04), (OLAPARIB:Metastatic malignant melanoma:0.17), (OLODATEROL HYDROCHLORIDETIOTROPIUM BROMIDE MONOHYDRATE:Metastatic malignant melanoma:0.57), (ONABOTULINUMTOXINA:Metastatic malignant melanoma:0.11), (ORPHENADRINE:Metastatic malignant melanoma:0.24), (OSIMERTINIB:Metastatic malignant melanoma:0.01), (PEGINTERFERON BETA-1A:Metastatic malignant melanoma:0.25), (PEMETREXED DISODIUM:Metastatic malignant melanoma:0.99), (PENICILLIN:Metastatic malignant melanoma:1.33), (PIMECROLIMUS:Metastatic malignant melanoma:0.29), (PIRFENIDONE:Metastatic malignant melanoma:0.05), (PONATINIB:Metastatic malignant melanoma:0.92), (PREDNISOLONE SODIUM PHOSPHATE:Metastatic malignant melanoma:0.13), (PRIMIDONE:Metastatic malignant melanoma:1.56), (PROGESTERONE:Metastatic malignant melanoma:0.45), (PROPAFENONE HYDROCHLORIDE:Metastatic malignant melanoma:0.15), (QUINAPRIL:Metastatic malignant melanoma:0.08), (RAMUCIRUMAB:Metastatic malignant melanoma:0.16), (RANOLAZINE:Metastatic malignant melanoma:1.16), (RIOCIGUAT:Metastatic malignant melanoma:0.08), (RIVASTIGMINE TARTRATE:Metastatic malignant melanoma:0.71), (ROFLUMILAST:Metastatic malignant melanoma:0.69), (SALMETEROL:Metastatic malignant melanoma:0.16), (SECUKINUMAB:Metastatic malignant melanoma:0.66), (SODIUM OXYBATE:Metastatic malignant melanoma:0.63), (SUMATRIPTANSUMATRIPTAN SUCCINATE:Metastatic malignant melanoma:0.79), (TERBUTALINE:Metastatic malignant melanoma:0.51), (TERBUTALINE SULFATE:Metastatic malignant melanoma:0.84), (TERIFLUNOMIDE:Metastatic malignant melanoma:0.36), (TETRACYCLINE:Metastatic malignant melanoma:1.07), (TICLOPIDINE:Metastatic malignant melanoma:0.09), (TIOTROPIUM:Metastatic malignant melanoma:1), (TIPIRACIL HYDROCHLORIDETRIFLURIDINE:Metastatic malignant melanoma:0.19), (TIZANIDINE HYDROCHLORIDE:Metastatic malignant melanoma:1.18), (TOCOPHEROL:Metastatic malignant melanoma:0.19), (TOLTERODINE TARTRATE:Metastatic malignant melanoma:0.72), (UMECLIDINIUM BROMIDE:Metastatic malignant melanoma:0.16), (UMECLIDINIUM BROMIDEVILANTEROL TRIFENATATE:Metastatic malignant melanoma:0.48), (VARENICLINE TARTRATE:Metastatic malignant melanoma:1.04), (VINORELBINE TARTRATE:Metastatic malignant melanoma:0.69), (ACETAMINOPHENCODEINE PHOSPHATE:Metastatic neoplasm:0.08), (AFATINIB:Metastatic neoplasm:0.04), (BISOPROLOL FUMARATE:Metastatic neoplasm:0.05), (BUMETANIDE:Metastatic neoplasm:0.38), (BUPROPION HYDROCHLORIDE:Metastatic neoplasm:0.05), (CANDESARTAN:Metastatic neoplasm:0.64), (CANDESARTAN CILEXETILHYDROCHLOROTHIAZIDE:Metastatic neoplasm:0.23), (COLCHICINE:Metastatic neoplasm:0.01), (DILTIAZEM:Metastatic neoplasm:0.03), (DILTIAZEM HYDROCHLORIDE:Metastatic neoplasm:0.42), (DRONABINOL:Metastatic neoplasm:0.22), (EPINEPHRINE:Metastatic neoplasm:0), (ERYTHROPOIETIN:Metastatic neoplasm:0.1), (FERROUS SULFATE:Metastatic neoplasm:0.05), (FILGRASTIM:Metastatic neoplasm:0.5), (GIMERACILOTERACILTEGAFUR:Metastatic neoplasm:0.03), (INSULIN ASPART:Metastatic neoplasm:0.07), (INTERFERON ALFA-2B:Metastatic neoplasm:0.04), (LEVOLEUCOVORIN CALCIUM:Metastatic neoplasm:0.37), (MELOXICAM:Metastatic neoplasm:0.03), (NAPROXEN:Metastatic neoplasm:0.01), (NITROFURANTOIN:Metastatic neoplasm:0.12), (OLAPARIB:Metastatic neoplasm:0.49), (PRAVASTATIN:Metastatic neoplasm:0.22), (RADIATION THERAPY:Metastatic neoplasm:0.49), (SACUBITRILVALSARTAN:Metastatic neoplasm:0.09), (VERAPAMIL HYDROCHLORIDE:Metastatic neoplasm:0.35), (FULVESTRANT:Metastatic pain:0.15), (ACARBOSE:Metastatic renal cell carcinoma:0.71), (ACETAMINOPHENCAFFEINE:Metastatic renal cell carcinoma:0.02), (ACETAMINOPHENCAFFEINEOPIUM:Metastatic renal cell carcinoma:0.01), (ACETAMINOPHENOXYCODONEOXYCODONE HYDROCHLORIDE:Metastatic renal cell carcinoma:0.69), (ACETAMINOPHENPROPOXYPHENE HYDROCHLORIDE:Metastatic renal cell carcinoma:0.13), (ACLIDINIUM BROMIDE:Metastatic renal cell carcinoma:0.5), (ADO-TRASTUZUMAB EMTANSINE:Metastatic renal cell carcinoma:1.13), (AFATINIB:Metastatic renal cell carcinoma:0.67), (AGALSIDASE BETA:Metastatic renal cell carcinoma:0.65), (ALENDRONIC ACID:Metastatic renal cell carcinoma:0.83), (ALISKIREN HEMIFUMARATE:Metastatic renal cell carcinoma:0.67), (AMISULPRIDE:Metastatic renal cell carcinoma:0.16), (AMLODIPINE BESYLATEHYDROCHLOROTHIAZIDEVALSARTAN:Metastatic renal cell carcinoma:0.44), (AMLODIPINEVALSARTAN:Metastatic renal cell carcinoma:0.68), (AMOXICILLINCLAVULANIC ACID:Metastatic renal cell carcinoma:0.49),(ARMODAFINIL:Metastatic renal cell carcinoma:0.58), (BACITRACIN:Metastatic renal cell carcinoma:0.26), (BEPRIDIL:Metastatic renal cell carcinoma:0.03), (BETAMETHASONE DIPROPIONATECALCIPOTRIENECALCIPOTRIENE HYDRATE:Metastatic renal cell carcinoma:0.05), (BORTEZOMIB:Metastatic renal cell carcinoma:0.58), (BUDESONIDEFORMOTEROL FUMARATE:Metastatic renal cell carcinoma:0.43), (BUPRENORPHINE HYDROCHLORIDENALOXONE HYDROCHLORIDE:Metastatic renal cell carcinoma:0.01), (CALCITONIN:Metastatic renal cell carcinoma:0.68), (CALCITONIN SALMON:Metastatic renal cell carcinoma:0.72), (CALCIUM CARBONATEVITAMIN D:Metastatic renal cell carcinoma:0.2), (CANAGLIFLOZINMETFORMIN HYDROCHLORIDE:Metastatic renal cell carcinoma:0.21), (CARBOXYMETHYLCELLULOSE SODIUM:Metastatic renal cell carcinoma:0.22), (CARFILZOMIB:Metastatic renal cell carcinoma:0.89), (CARVEDILOL PHOSPHATE:Metastatic renal cell carcinoma:1.32), (CEFEPIMECEFEPIME HYDROCHLORIDE:Metastatic renal cell carcinoma:0.05), (CEFPODOXIME PROXETIL:Metastatic renal cell carcinoma:0.06), (CERTOLIZUMAB PEGOL:Metastatic renal cell carcinoma:1.13), (CHLORHEXIDINE:Metastatic renal cell carcinoma:0.28), (CICLESONIDE:Metastatic renal cell carcinoma:0.13), (CILASTATIN SODIUMIMIPENEM:Metastatic renal cell carcinoma:0.51), (CORTICOTROPIN:Metastatic renal cell carcinoma:0.05), (DABIGATRAN:Metastatic renal cell carcinoma:0.87), (DABRAFENIB:Metastatic renal cell carcinoma:0.69), (DABRAFENIB MESYLATE:Metastatic renal cell carcinoma:0.23), (DACLIZUMAB:Metastatic renal cell carcinoma:0.03), (DANAZOL:Metastatic renal cell carcinoma:0.58), (DAPAGLIFLOZIN PROPANEDIOL:Metastatic renal cell carcinoma:0.07), (DASATINIB:Metastatic renal cell carcinoma:1.75), (DEFLAZACORT:Metastatic renal cell carcinoma:0.94), (DEGARELIX:Metastatic renal cell carcinoma:0.29), (DEVICE:Metastatic renal cell carcinoma:0.08), (DEXAMETHASONE ACETATE:Metastatic renal cell carcinoma:0.39), (DOFETILIDE:Metastatic renal cell carcinoma:0.67), (DRONEDARONE:Metastatic renal cell carcinoma:0.54), (ELETRIPTAN HYDROBROMIDE:Metastatic renal cell carcinoma:0.68), (ELOTUZUMAB:Metastatic renal cell carcinoma:0.33), (EPIRUBICIN:Metastatic renal cell carcinoma:0.16), (ERIBULIN:Metastatic renal cell carcinoma:0.74), (ESTROGENS, CONJUGATEDMEDROXYPROGESTERONE ACETATE:Metastatic renal cell carcinoma:0.28), (ETRETINATE:Metastatic renal cell carcinoma:0.34), (EVOLOCUMAB:Metastatic renal cell carcinoma:0.56), (EXEMESTANE:Metastatic renal cell carcinoma:1.19), (FERROUS SULFATEGLYCINE:Metastatic renal cell carcinoma:0.22), (FESOTERODINE FUMARATE:Metastatic renal cell carcinoma:1.1), (FLUOCINONIDE:Metastatic renal cell carcinoma:0.63), (FLUTICASONE FUROATE:Metastatic renal cell carcinoma:0.42), (FLUVOXAMINE MALEATE:Metastatic renal cell carcinoma:0.12), (FORMOTEROL:Metastatic renal cell carcinoma:0.45), (FORMOTEROL FUMARATE DIHYDRATEMOMETASONE FUROATE:Metastatic renal cell carcinoma:0.64), (FULVESTRANT:Metastatic renal cell carcinoma:0.59), (GLYCERIN:Metastatic renal cell carcinoma:0.28), (GLYCOPYRROLATE:Metastatic renal cell carcinoma:0.09), (HYDROCODONE BITARTRATEIBUPROFEN:Metastatic renal cell carcinoma:0.09), (HYDROXYCHLOROQUINE DIPHOSPHATE:Metastatic renal cell carcinoma:0.24), (HYOSCYAMINE:Metastatic renal cell carcinoma:0.81), (IBRUTINIB:Metastatic renal cell carcinoma:0.68), (ILOPROST:Metastatic renal cell carcinoma:0.55), (IMIPRAMINE HYDROCHLORIDE:Metastatic renal cell carcinoma:0.48), (INDACATEROL:Metastatic renal cell carcinoma:0.23), (INFLIXIMAB-DYYB: Metastatic renal cell carcinoma:0.13), (IPRATROPIUM:Metastatic renal cell carcinoma:1.53), (IRINOTECAN HYDROCHLORIDE:Metastatic renal cell carcinoma:0.93), (ITRACONAZOLE:Metastatic renal cell carcinoma:0.16), (LACTASE:Metastatic renal cell carcinoma:0.28), (LANREOTIDE:Metastatic renal cell carcinoma:0.1), (LEDIPASVIRSOFOSBUVIR:Metastatic renal cell carcinoma:0.96), (LENALIDOMIDE:Metastatic renal cell carcinoma:1.03), (LEVALBUTEROL HYDROCHLORIDE:Metastatic renal cell carcinoma:1.29), (LEVOLEUCOVORIN CALCIUM:Metastatic renal cell carcinoma:0.55), (LIOTHYRONINE SODIUM:Metastatic renal cell carcinoma:0.03), (LISDEXAMFETAMINE DIMESYLATE:Metastatic renal cell carcinoma:0.26), (LOMITAPIDE MESYLATE:Metastatic renal cell carcinoma:0.17), (METHENAMINE HIPPURATE:Metastatic renal cell carcinoma:0.41), (METHYLPREDNISOLONE ACETATE:Metastatic renal cell carcinoma:0.95), (MINOCYCLINEMINOCYCLINE HYDROCHLORIDE:Metastatic renal cell carcinoma:1.22), (MODAFINIL:Metastatic renal cell carcinoma:0.3), (NINTEDANIB:Metastatic renal cell carcinoma:0.78), (NIRAPARIB:Metastatic renal cell carcinoma:0.71), (OLAPARIB:Metastatic renal cell carcinoma:0.43), (OLODATEROL HYDROCHLORIDETIOTROPIUM BROMIDE MONOHYDRATE:Metastatic renal cell carcinoma:0.42), (OSIMERTINIB:Metastatic renal cell carcinoma:0.47), (OXCARBAZEPINE:Metastatic renal cell carcinoma:1.72), (PANITUMUMAB:Metastatic renal cell carcinoma:0.36), (PATIROMER:Metastatic renal cell carcinoma:0.26), (PEGINTERFERON BETA-1A:Metastatic renal cell carcinoma:0.05), (PEMETREXED:Metastatic renal cell carcinoma:0.83), (PEMETREXED DISODIUM:Metastatic renal cell carcinoma:0.87), (PENICILLIN:Metastatic renal cell carcinoma:0.93), (PENTOSAN POLYSULFATE SODIUM:Metastatic renal cell carcinoma:0.03), (PERTUZUMAB:Metastatic renal cell carcinoma:0.19), (PILOCARPINE:Metastatic renal cell carcinoma:0.03), (PIMOBENDAN:Metastatic renal cell carcinoma:0.09), (PIROXICAM:Metastatic renal cell carcinoma:0.42), (POLYETHYLENE GLYCOL 3350POTASSIUM CHLORIDE\SODIUM BICARBONATE\SODIUM CHLORIDE\SODIUM SULFATE ANHYDROUS:Metastatic renal cell carcinoma:0.03), (PONATINIB:Metastatic renal cell carcinoma:0.72), (PREDNISOLONE ACETATE:Metastatic renal cell carcinoma:0.66), (PYRIDOSTIGMINE BROMIDE:Metastatic renal cell carcinoma:0.17), (QUETIAPINE:Metastatic renal cell carcinoma:1.28), (RETINOL:Metastatic renal cell carcinoma:0.45), (RIFAXIMIN:Metastatic renal cell carcinoma:0.05), (RIVASTIGMINE:Metastatic renal cell carcinoma:0.27), (RIVASTIGMINE TARTRATE:Metastatic renal cell carcinoma:0.37), (RIZATRIPTAN BENZOATE:Metastatic renal cell carcinoma:0.36), (ROPINIROLE HYDROCHLORIDE:Metastatic renal cell carcinoma:0.71), (SACCHAROMYCES CEREVISIAE:Metastatic renal cell carcinoma:0.25), (SACUBITRILVALSARTAN:Metastatic renal cell carcinoma:0.06), (SARPOGRELATE:Metastatic renal cell carcinoma:0.48), (SECUKINUMAB:Metastatic renal cell carcinoma:0.41), (SIPULEUCEL-T: Metastatic renal cell carcinoma:0.01), (SODIUM OXYBATE:Metastatic renal cell carcinoma:0.74), (SODIUM PHOSPHATE, DIBASIC\SODIUM PHOSPHATE, MONOBASIC:Metastatic renal cell carcinoma:0.34), (SULFAMETHOXAZOLE:Metastatic renal cell carcinoma:0.58), (SUMATRIPTAN:Metastatic renal cell carcinoma:0.21), (SUMATRIPTANSUMATRIPTAN SUCCINATE:Metastatic renal cell carcinoma:0.21), (TELOTRISTAT ETHYL:Metastatic renal cell carcinoma:0.07), (TERBUTALINE:Metastatic renal cell carcinoma:0.01), (TESTOSTERONE CYPIONATE:Metastatic renal cell carcinoma:0.01), (TETRACYCLINE:Metastatic renal cell carcinoma:1.1), (TICLOPIDINE:Metastatic renal cell carcinoma:0.07), (TIPIRACIL HYDROCHLORIDETRIFLURIDINE:Metastatic renal cell carcinoma:0.31), (TOCILIZUMAB:Metastatic renal cell carcinoma:0.07), (TRAMETINIB:Metastatic renal cell carcinoma:0.64), (TRAMETINIB DIMETHYL SULFOXIDE:Metastatic renal cell carcinoma:0.11), (TROSPIUM CHLORIDE:Metastatic renal cell carcinoma:0.34), (UMECLIDINIUM BROMIDE:Metastatic renal cell carcinoma:0.02), (UMECLIDINIUM BROMIDEVILANTEROL TRIFENATATE:Metastatic renal cell carcinoma:0.74), (VALGANCICLOVIR HYDROCHLORIDE:Metastatic renal cell carcinoma:1.05), (VANCOMYCIN HYDROCHLORIDE:Metastatic renal cell carcinoma:0.35), (VEMURAFENIB:Metastatic renal cell carcinoma:0.69), (VILAZODONE HYDROCHLORIDE:Metastatic renal cell carcinoma:0.03), (VINORELBINE TARTRATE:Metastatic renal cell carcinoma:0.69), (VITAMIN A:Metastatic renal cell carcinoma:0.23), (ARMODAFINIL:Micturition disorder:0.27), (CALCIUMVITAMIN D:Micturition disorder:0.26), (CANDESARTAN:Micturition disorder:0.33), (CANDESARTAN CILEXETIL:Micturition disorder:0.35), (CANDESARTAN CILEXETILHYDROCHLOROTHIAZIDE:Micturition disorder:0.05), (CERTOLIZUMAB PEGOL:Micturition disorder:0.24), (DICLOFENAC:Micturition disorder:0.32), (DILTIAZEM HYDROCHLORIDE:Micturition disorder:0.92), (ELETRIPTAN HYDROBROMIDE:Micturition disorder:0.04), (EVEROLIMUS:Micturition disorder:0.01), (EVOLOCUMAB:Micturition disorder:0.81), (FEBUXOSTAT:Micturition disorder:0.02), (FLECAINIDE:Micturition disorder:0.2), (INDAPAMIDE:Micturition disorder:0.1), (INDAPAMIDEPERINDOPRIL:Micturition disorder:0.02), (LEDIPASVIRSOFOSBUVIR:Micturition disorder:0.11), (LIDOCAINE:Micturition disorder:0.1), (LINAGLIPTIN:Micturition disorder:0.13), (LISDEXAMFETAMINE DIMESYLATE:Micturition disorder:0.15), (METAXALONE:Micturition disorder:0.22), (METHYLPREDNISOLONE SODIUM SUCCINATE:Micturition disorder:0.58), (METRONIDAZOLE:Micturition disorder:0.52), (MORPHINE:Micturition disorder:0.53), (NITROFURANTOIN:Micturition disorder:0.1), (NORTRIPTYLINE:Micturition disorder:0.02), (OMEGA-3-ACID ETHYL ESTERS:Micturition disorder:0.2), (PRAMIPEXOLE DIHYDROCHLORIDE:Micturition disorder:0.5), (PRAVASTATIN SODIUM:Micturition disorder:0.7), (PROMETHAZINEPROMETHAZINE HYDROCHLORIDE:Micturition disorder:0.18), (TIMOLOL:Micturition disorder:0.13), (TOFACITINIB CITRATE:Micturition disorder:0.3), (ARMODAFINIL:Micturition urgency:1.09), (ATROPINE SULFATEDIPHENOXYLATE HYDROCHLORIDE:Micturition urgency:0.31), (BISACODYL OR DOCUSATE SODIUM:Micturition urgency:0.74), (CANDESARTAN CILEXETILHYDROCHLOROTHIAZIDE:Micturition urgency:0.53), (CARFILZOMIB:Micturition urgency:0.24), (CARISOPRODOL:Micturition urgency:0.57), (CARVEDILOL PHOSPHATE:Micturition urgency:0.47), (CERTOLIZUMAB PEGOL:Micturition urgency:0.98), (CIMETIDINE:Micturition urgency:0.09), (CLINDAMYCINCLINDAMYCIN PHOSPHATE:Micturition urgency:0.63), (CLOTRIMAZOLE:Micturition urgency:0.3), (DAPAGLIFLOZIN PROPANEDIOL:Micturition urgency:0.5), (DASATINIB:Micturition urgency:0.4), (DIVALPROEX SODIUM:Micturition urgency:0.48), (DOFETILIDE:Micturition urgency:0.09), (DRONABINOL:Micturition urgency:0.24), (ENALAPRIL MALEATE:Micturition urgency:0.78), (EPINEPHRINE:Micturition urgency:0.6), (ESTROGENS, CONJUGATEDMEDROXYPROGESTERONE ACETATE:Micturition urgency:0.56), (EVEROLIMUS:Micturition urgency:0.42), (EVOLOCUMAB:Micturition urgency:1.1), (FEBUXOSTAT:Micturition urgency:0.36), (FENTANYL CITRATE:Micturition urgency:0.11), (FLECAINIDE:Micturition urgency:0.33), (FLUOXETINEFLUOXETINE HYDROCHLORIDE:Micturition urgency:0.3), (FLUTICASONE FUROATEVILANTEROL TRIFENATATE:Micturition urgency:0.22), (HYDROCHLOROTHIAZIDEIRBESARTAN:Micturition urgency:0.44), (HYDROCHLOROTHIAZIDETELMISARTAN:Micturition urgency:0.22), (HYDROXYCHLOROQUINE DIPHOSPHATE:Micturition urgency:0.33), (HYDROXYZINE PAMOATE:Micturition urgency:0.88), (INDAPAMIDEPERINDOPRIL:Micturition urgency:0.12), (INSULIN GLULISINE:Micturition urgency:0.07), (ISOSORBIDE:Micturition urgency:0.42), (LABETALOLLABETALOL HYDROCHLORIDE:Micturition urgency:0.23), (LETROZOLE:Micturition urgency:0.03), (METHYLPREDNISOLONE ACETATE:Micturition urgency:0.59), (METRONIDAZOLEMETRONIDAZOLE HYDROCHLORIDE:Micturition urgency:0.29), (MOXIFLOXACIN HYDROCHLORIDE:Micturition urgency:0.23), (NALOXONE HYDROCHLORIDEOXYCODONE HYDROCHLORIDE:Micturition urgency:0.28), (NEBIVOLOL:Micturition urgency:0.33), (NILOTINIB:Micturition urgency:0.25), (OLANZAPINE:Micturition urgency:0.76), (PANTOPRAZOLE:Micturition urgency:1.44), (PANTOPRAZOLE MAGNESIUM:Micturition urgency:0.48), (PAZOPANIB HYDROCHLORIDE:Micturition urgency:0.15), (PENICILLIN:Micturition urgency:0.18), (PROPRANOLOLPROPRANOLOL HYDROCHLORIDE:Micturition urgency:0.17), (QUETIAPINE:Micturition urgency:0.58), (RANOLAZINE:Micturition urgency:0.8), (RIVASTIGMINE TARTRATE:Micturition urgency:0.26), (ROFLUMILAST:Micturition urgency:0.15), (SACUBITRILVALSARTAN:Micturition urgency:0.97), (SUCRALFATE:Micturition urgency:0.28), (SUMATRIPTAN:Micturition urgency:0.24), (SUMATRIPTANSUMATRIPTAN SUCCINATE:Micturition urgency:0.72), (TIMOLOL:Micturition urgency:0.38), (VILAZODONE HYDROCHLORIDE:Micturition urgency:0.21), (ZINC:Micturition urgency:0.2), (ZOLEDRONIC ACID:Micturition urgency:0.27), (ZONISAMIDE:Micturition urgency:0.11), (.ALPHA.-TOCOPHEROLVITAMINS:Migraine:0.09), (ACARBOSE:Migraine:0.97), (ACITRETIN:Migra ine:0.96), (AJMALINE:Migraine:0.45), (ALBIGLUTIDE:Migraine:0.74), (ALLANTOINBENZ OCAINECAMPHOR (NATURAL)DIMETHICONEMENTHOL:Migraine:0.1), (ALUMINUM HYDROXIDECALCIUM CARBONATEDIMETHICONEMAGNESIUM HYDROXIDE:Migraine:0.14), (ALUMINUM HYDROXIDEMAGNESIUM HYDROXIDESIMETHICONE:Migraine:0.3), (AMILORIDE HYDROCHLORIDEFUROSEMIDE:Migraine:0.09), (AMINO ACIDSDEXTROSEELECTROLYTES NOS:Migraine:1.62), (AMISULPRIDE:Migraine:1.32), (AMLODIPINE BESYLATEHYDROCHLOROTHIAZIDEVALSARTAN:Migraine:1.1), (AMLODIPINEPER INDOPRIL:Migraine:0.12), (AMLODIPINEVALSARTAN:Migraine:1.15), (AMOXICILLINC LARITHROMYCINESOMEPRAZOLE:Migraine:0.73), (AMPHETAMINE ASPARTATEDEXTROAMPHETAMINE SACCHARATE:Migraine:0.15), (AMPHETAMINE SULFATEDEXTROAMPHETAMINE:Migraine:0.21), (ANAGRELIDE:Migraine:0.01), (ASC ORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Migraine:0.34), (ASCORBIC ACIDBIOTINCYANOCOBALAMINFOLIC ACIDNIACINPANTOTHENIC ACIDPYRIDOXINE\RIBOFLAVINTHIAMINE:Migraine:0.08), (ASPIRIN\CAFFEINESALI CYLAMIDE:Migraine:0.67), (ASPIRINMAGNESIUM OXIDE:Migraine:0.41), (AZELNIDIPINE:Migraine:0), (BAMIFYLLINE HYDROCHLORIDE:Migraine:0.29), (BECLOMETHASONE:Migraine:1.33), (BEE POLLEN:Migraine:0.06), (BENZOCAINE:Migraine:0.44), (BENZOCAINERESORCINOL:Mig raine:0.02), (BENZOIC ACIDHYOSCYAMINE SULFATEMETHENAMINEMETHYLENE BLUEPHENYL SALICYLATE:Migraine:0.29), (BETAMETHASONE DIPROPIONATECALCIPOTRIENE:Migraine:0.78), (BETAMETHASONE SODIUM PHOSPHATE:Migraine:0.32), (BICALUTAMIDE:Migraine:1.38), (BICARBONATE ION:Migraine:0.07), (BLINATUMOMAB:Migraine:0.83), (BORIC ACID:Migraine:0.28), (BORON:Migraine:0.18), (BRIVARACETAM:Migraine:0.9), (CABOZAN TINIB S-MALATE:Migraine:0.52), (CAJUPUT OILEUCALYPTUS OILPEPPERMINT OIL:Migraine:0.09), (CALCIUM CARBONATEMAGNESIUM HYDROXIDE:Migraine:0.01), (CALCIUM PANTOTHENATECYANOCOBALAMINNIACINAMIDEPYRIDOXINE HYDROCHLORIDE\RIBOFLAVINTHIAMINE MONONITRATE:Migraine:0.12), (CAMPHORDIPHENHYDRAMINE\ZINC OXIDE:Migraine:0.34), (CANNABIDIOL:Migraine:0.28), (CARBAMIDE PEROXIDE:Migraine:0.24), (CARNITINE:Migraine:0.11), (CEFOTAXIME SODIUM:Migraine:0.61), (CEFPROZIL:Migraine:0.85), (CETUXIMAB:Migraine:0.7), (CHLOP HEDIANOL HYDROCHLORIDEDEXBROMPHENIRAMINE MALEATEPHENYLEPHRINE HYDROCHLORIDE:Migraine:0), (CHLORMADINONE ACETATE:Migraine:0.04), (CHLORMADINONE ACETATEETHINYL ESTRADIOL:Migraine:0.33), (CHLOROTHIAZIDE:Migraine:0.81), (CHLORPROTHIXENE:M igraine:0.42), (CICLOPIROX OLAMINE:Migraine:0.45), (CILASTATIN SODIUMIMIPENEM:Migraine:0.83), (CLIKSTAR:Migraine:0.48), (COBICISTATELVITEGR AVIREMTRICITABINETENOFOVIR ALAFENAMIDE FUMARATE:Migraine:0.85), (COLISTIMETHATE SODIUM:Migraine:0.1), (DABIGATRAN:Migraine:0.89), (DABRAFENIB MESYLATE:Migraine:0.24), (DAPAGLIFLOZIN PROPANEDIOLMETFORMIN HYDROCHLORIDE:Migraine:0.35), (DARATUMUMAB:Migraine:0.09), (DEXAMETHASON E ACETATE:Migraine:0.56), (DEXPANTHENOLNIACINAMIDEPYRIDOXINE HYDROCHLORIDE\RIBOFLAVIN 5′-PHOSPHATE SODIUMTHIAMINE HYDROCHLORIDE:Migraine:0.7), (DICLOXACILLIN:Migraine:0.59), (DIENOGEST:Migrain e:1.1), (DIETARY SUPPLEMENTRESVERATROL:Migraine:0.16), (DIGITOXIN:Migraine:0.08), (DIHYDROCO DEINE PHOSPHATE:Migraine:0.06), (DIMETHYL SULFOXIDE:Migraine:0.42), (DIOSMIN:Migraine:0.37), (DIPHENHYDRAMINE HYDROCHLORIDEDOXYLAMINE SUCCINATE:Migraine:0.23), (DOXYCYCLINE HYDROCHLORIDE:Migraine:0.07), (DYDROGESTERONE:Migraine:0.2), (DYDROGESTER ONEESTRADIOL:Migraine:0.01), (ELDECALCITOL:Migraine:0.07), (ELOTUZUMAB:Migra ine:0.77), (EMPAGLIFLOZINLINAGLIPTIN:Migraine:1.56), (EMTRICITABINERILPIVIRIN ETENOFOVIR DISOPROXIL FUMARATE:Migraine:0.02), (EMTRICITABINETENOFOVIR ALAFENAMIDE FUMARATE:Migraine:0.31), (ENSULIZOLEEUCERINOCTINOXATEOCTISALATETITA NIUM DIOXIDE\ZINC OXIDE:Migraine:0.32), (EPHEDRINE HYDROCHLORIDEGUAIFENESIN:Migraine:0.68), (ERTAPENEM SODIUM:Migraine:0.23), (ESTRADIOL BENZOATE:Migraine:1.34), (ETHOSUXIMIDE:Migraine:0.68), (ETRETINATE:Migraine:0.33), (FERRIC POLYSACCHARIDE COMPLEX:Migraine:0.41), (FERRITIN:Migraine:0.03), (FERROUS BISGLYCINATE:Migraine:1.24), (FERROUS SULFATEFOLIC ACID:Migraine:0.23), (FLUINDIONE:Migraine:0.6), (FLUNARIZINE HYDROCHLORIDE:Migraine:0.23), (FLUOCINOLONE:Migraine:0.27), (FLUPHENAZINE:M igraine:0.33), (FLUPHENAZINE HYDROCHLORIDE:Migraine:0.18), (GARLIC OIL:Migraine:0.12), (GIMERACILOTERACILTEGAFUR:Migraine:0.52), (GINSENG EXTRACT:Migraine:0.06), (GLUTAMINE:Migraine:0.68), (GLYBURIDEMETFORMIN HYDROCHLORIDE:Migraine:0.18), (GLYCOPYRROLATEINDACATEROL MALEATE:Migraine:0.62), (GLYCOPYRRONIUM:Migraine:1.12), (GLYCOPYRRONIUMIN DACATEROL:Migraine:1.63), (GREEN TEA LEAF:Migraine:0.03), (GUAIFENESINPHENYLEPHRINE HYDROCHLORIDE:Migraine:0.25), (HERBALSMINERALSVITAMINS:Migraine:0.65), (HI GH DENSITY POLYETHYLENE:Migraine:0.38), (HUMAN PAPILLOMAVIRUS QUADRIVALENT (TYPES 6, 11, 16, AND 18) VACCINE, RECOMBINANT:Migraine:0.17), (HYDROCHLOROTHIAZIDESPIRONOLACTONE:Migrai ne:0.32), (HYDROCORTISONE VALERATE:Migraine:0.09), (HYDROQUINONE:Migraine:0.39), (HYPERICUM PERFORATUM:Migraine:0.22), (IBRITUMOMAB TIUXETAN:Migraine:0.09), (INDAPAMIDEPERINDOPRIL:Migraine:1.61), (INOTUZUMAB OZOGAMICIN:Migraine:0.12), (INTERFERON:Migraine:1.76), (INTERFERON ALFA-2A:Migraine:0.97), (INTERFERON GAMMA-1B:Migraine:0.94), (IODINE\POTASSIUM IODIDE:Migraine:0.2), (IRINOTECAN:Migraine:0.15), (IRINOTECAN HYDROCHLORIDE:Migraine:1.03), (ITRACONAZOLE:Migraine:1.31), (ITRACONAZOLES ODIUM CHLORIDE:Migraine:0.4), (IXEKIZUMAB:Migraine:0.15), (LANREOTIDE:Migraine:0.35), (L APATINIB DITOSYLATE:Migraine:0.83), (LECITHIN:Migraine:0.8), (LENVATINIB:Migraine:0.33), (LER CANIDIPINE:Migraine:1.77), (LEVOLEUCOVORIN CALCIUM:Migraine:0.47), (LINEZOLID:Migraine:0.06), (LUTETIUM LU-177:Migraine:0.45), (MAGNESIUM SULFATE\POTASSIUM SULFATE\SODIUM SULFATE:Migraine:0.12), (MANGANESEMANGANESE CHLORIDE:Migraine:0.05), (MATRICARIA RECUTITA:Migraine:0.28), (MENTHOLZINC OXIDE:Migraine:0.37), (MESNA:Migraine:0.63), (MESTRANOLNORETHINDRONE:Migrain e:1.09), (METFORMIN HYDROCHLORIDESAXAGLIPTIN HYDROCHLORIDE:Migraine:0.52), (METHENAMINE:Migraine:0.83), (METHENAMINE MANDELATE:Migraine:0.88), (METHSUXIMIDE:Migraine:0.1), (METRELEPTIN:Migraine:00.74), (MOEXIPRIL HYDROCHLORIDE:Migraine:1.28), (MOXONIDINE:Migraine:0.48), (NALOXONE:Migraine: 0.81), (NALOXONE HYDROCHLORIDETILIDINE HYDROCHLORIDE:Migraine:1.14), (NEFAZODONE:Migraine:0.95), (NIACINAMIDE:Migrai ne:0.27), (NOREPINEPHRINE:Migraine:0.07), (OMEGA-6 FATTY ACIDS:Migraine:0.47), (OSELTAMIVIR:Migraine:0.9), (OXYQUINOLINE SULFATE\SODIUM LAURYL SULFATE:Migraine:0.09), (PANITUMUMAB:Migraine:0.26), (PAPAYA:Migraine:0.63), (PASI REOTIDE:Migraine:0.39), (PEMETREXED:Migraine:0.58), (PEMETREXED DISODIUM:Migraine:0.88), (PHENPROCOUMON:Migraine:0.2), (PHENYLEPHRINE HYDROCHLORIDEPROMETHAZINE HYDROCHLORIDE:Migraine:0.3), (PINDOLOL:Migraine:0.49), (PNEUMOCOCCAL VACCINE POLYVALENT:Migraine:0.32), (PRAMLINTIDE ACETATE:Migraine:0.62), (PROCAINE:Migraine:0.15), (PRUCALOPRIDE SUCCINATE:Migraine:0.13), (QUINIDINE GLUCONATE:Migraine:0.55), (RADIATION THERAPY:Migraine:1.14), (RAMUCIRUMAB:Migraine:0.15), (RIOCIGUAT:Migraine:0.73), (RUFINAMIDE:Migraine:0.48), (SAPROPTERIN DIHYDROCHLORIDE:Migraine:0.69), (SELEGILINE HYDROCHLORIDE:Migraine:0.11), (SEVELAMER HYDROCHLORIDE:Migraine:0.27), (SILICON DIOXIDE:Migraine:0.06), (SITAGLIPTIN:Migraine:1.25), (SODIUM CITRATE:Migraine:0.06), (SODIUM FERROUS CITRATE:Migraine:0.13), (SODIUM FLUORIDEVITAMINS:Migraine:0.27), (SODIUM HYPOCHLORITE:Migraine:0.06), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Migraine:0.1), (SUFENTANIL CITRATE:Migraine:0.44), (SULFAMETHOXAZOLE:Migraine:2.08), (TELOTRISTAT ETHYL:Migraine:0.84), (TESTOSTERONE UNDECANOATE:Migraine:1.16), (THEANINE:Migraine:0.46), (TIMOLOL MALEATETRAVOPROST:Migraine:0.32), (TINIDAZOLE:Migraine:0.55), (TIOCONAZOLE: Migraine:0.22), (TIOPRONIN:Migraine:0.29), (TRAMETINIB DIMETHYL SULFOXIDE:Migraine:0.26), (TRANYLCYPROMINE SULFATE:Migraine:0.91), (TRIENTINE HYDROCHLORIDE:Migraine:0), (TRIPTORELIN PAMOATE:Migraine:0.74), (TROSPIUM:Migraine:0.17), (VALDECOXIB:Migraine:0.45), (VA NCOMYCIN HYDROCHLORIDE:Migraine:0.76), (WARFARIN POTASSIUM:Migraine:0.03), (ZIPRASIDONE HYDROCHLORIDE:Migraine:0.55), (AMIODARONE:Migraine prophylaxis:0.22), (AZITHROMYCIN DIHYDRATE:Migraine prophylaxis:0.05), (BISACODYL OR DOCUSATE SODIUM:Migraine prophylaxis:0.25), (BUPRENORPHINE HYDROCHLORIDE:Migraine prophylaxis:0.21), (BUPRENORPHINE HYDROCHLORIDENALOXONE HYDROCHLORIDE:Migraine prophylaxis:0.44), (CALCIUM CARBONATECHOLECALCIFEROL:Migraine prophylaxis:0.64), (CANNABIS SATIVA SUBSP. INDICA TOP:Migraine prophylaxis:0.01), (CERTOLIZUMAB PEGOL:Migraine prophylaxis:0.93), (CLOTRIMAZOLE:Migraine prophylaxis:0.12), (COLCHICINE:Migraine prophylaxis:0.27), (COLESEVELAM HYDROCHLORIDE:Migraine prophylaxis:0.03), (DOXEPIN HYDROCHLORIDE:Migraine prophylaxis:0.02), (DRONABINOL:Migraine prophylaxis:0.01), (ENALAPRIL MALEATE:Migraine prophylaxis:0.3), (ESTRADIOLNORETHINDRONE ACETATE:Migraine prophylaxis:0.01), (EVOLOCUMAB:Migraine prophylaxis:0.89), (GLIMEPIRIDE:Migraine prophylaxis:0.7), (HERBALSTURMERIC:Migraine prophylaxis:0.01), (IMIPRAMINE HYDROCHLORIDE:Migraine prophylaxis:0.07), (INSULIN HUMAN:Migraine prophylaxis:0.38), (IRBESARTAN:Migraine prophylaxis:0.03), (ISOSORBIDE MONONITRATE:Migraine prophylaxis:0.02), (METHENAMINE HIPPURATE:Migraine prophylaxis:0.01), (METHYLPREDNISOLONE ACETATE:Migraine prophylaxis:0.6), (METRONIDAZOLEMETRONIDAZOLE HYDROCHLORIDE:Migraine prophylaxis:0.29), (MOXIFLOXACIN HYDROCHLORIDE:Migraine prophylaxis:0.25), (NIACIN:Migraine prophylaxis:0.27), (NICOTINE:Migraine prophylaxis:0.18), (OXYBUTYNIN CHLORIDE:Migraine prophylaxis:0.23), (PANTOPRAZOLE MAGNESIUM:Migraine prophylaxis:0.27), (PAROXETINE:Migraine prophylaxis:0.46), (PHENOBARBITAL:Migraine prophylaxis:0.42), (POLYETHYLENE GLYCOLS:Migraine prophylaxis:0.32), (PRAMIPEXOLE:Migraine prophylaxis:0.34), (RAMIPRIL:Migraine prophylaxis:0.72), (RANOLAZINE:Migraine prophylaxis:0.58), (RISEDRONATE SODIUM:Migraine prophylaxis:0.2), (SACUBITRILVALSARTAN:Migraine prophylaxis:0.19), (SECUKINUMAB:Migraine prophylaxis:0.05), (TERIFLUNOMIDE:Migraine prophylaxis:1.03), (TOCILIZUMAB:Migraine prophylaxis:0.2), (TOLTERODINE TARTRATE:Migraine prophylaxis:0.19), (TRIAMCINOLONE ACETONIDE:Migraine prophylaxis:0.43), (TRIAMTERENE:Migraine prophylaxis:0.07), (VITAMIN B:Migraine prophylaxis:0.83), (ZINC:Migraine prophylaxis:0.1), (.ALPHA.1-PROTEINASE INHIBITOR HUMAN:Mineral supplementation:1.68), (.BETA.-HYDROXYISOVALERIC ACIDARGININEGLUTAMINE:Mineral supplementation:0.06), (ABOBOTULINUMTOXINA:Mineral supplementation:0.01), (ALGLUCOSIDASE ALFA:Mineral supplementation:0.07), (ALOGLIPTIN BENZOATE:Mineral supplementation:0.36), (AMIKACIN SULFATE:Mineral supplementation:0.44), (AMILORIDE HYDROCHLORIDEHYDROCHLOROTHIAZIDE:Mineral supplementation:1.53), (AMINO ACIDSDEXTROSEELECTROLYTES NOS:Mineral supplementation:1.73), (AMLODIPINE BESYLATEHYDROCHLOROTHIAZIDEVALSARTAN:Mineral supplementation:1.34), (AMMONIUM CHLORIDEDEXTROMETHORPHAN\SODIUM CITRATE:Mineral supplementation:0.32), (AMOXICILLINCLARITHROMYCINESOMEPRAZOLE:Mineral supplementation:0.27), (ANAGRELIDE:Mineral supplementation:0.1), (ANAGRELIDE HYDROCHLORIDE:Mineral supplementation:0.66), (ANHYDROUS CITRIC ACIDASPIRIN\SODIUM BICARBONATE:Mineral supplementation:0.35), (ANTIHISTAMINES NOS:Mineral supplementation:0.94), (ARIPIPRAZOLE LAUROXIL:Mineral supplementation:0.23), (ASCORBIC ACIDBIOTINCYANOCOBALAMINFOLIC ACIDNIACINPANTOTHENIC ACIDPYRIDOXINE\RIBOFLAVINTHIAMINE:Mineral supplementation:0.31), (ASIAN GINSENG:Mineral supplementation:0.09), (BACITRACIN:Mineral supplementation:1.19), (BAMIFYLLINE HYDROCHLORIDE:Mineral supplementation:0.17), (BAZEDOXIFENE ACETATEESTROGENS, CONJUGATED:Mineral supplementation:0.07), (BECLOMETHASONE:Mineral supplementation:0.83), (BERAPROST:Mineral supplementation:0.28), (BETAMETHASONECALCIPOTRIENE:Mineral supplementation:1.1), (BETAXOLOL HYDROCHLORIDE:Mineral supplementation:0.23), (BIFIDOBACTERIUM ANIMALIS LACTIS:Mineral supplementation:0.13), (BILASTINE:Mineral supplementation:0.05), (BLESELUMAB:Mineral supplementation:0.44), (BLINATUMOMAB:Mineral supplementation:1.01), (BOCEPREVIR:Mineral supplementation:0.35), (BOSUTINIB:Mineral supplementation:0.08), (BOTULINUM TOXIN TYPE A:Mineral supplementation:0.12), (BROMHEXINE:Mineral supplementation:0.12), (BUPIVACAINE HYDROCHLORIDE:Mineral supplementation:0.16), (CABOZANTINIB:Mineral supplementation:0), (CABOZANTINIB S-MALATE:Mineral supplementation:0.47), (CAMPHORDIPHENHYDRAMINE\ZINC OXIDE:Mineral supplementation:0.03), (CANAKINUMAB:Mineral supplementation:1.5), (CARTEOLOL HYDROCHLORIDE:Mineral supplementation:0.13), (CARVEDILOL PHOSPHATE:Mineral supplementation:3.24), (CEFEPIMECEFEPIME HYDROCHLORIDE:Mineral supplementation:0.87), (CEFOTAXIMECEFOTAXIME SODIUM:Mineral supplementation:0.04), (CERIVASTATIN SODIUM:Mineral supplementation:0.49), (CERTOPARIN SODIUM:Mineral supplementation:0.26), (CHOLESTEROL:Mineral supplementation:0.25), (CICLOPIROX:Mineral supplementation:0.32), (CICLOPIROX OLAMINE:Mineral supplementation:0.4), (CINACALCET:Mineral supplementation:0.42), (CISATRACURIUM:Mineral supplementation:0.27), (CLOBAZAM:Mineral supplementation:1.43), (CLOMIPRAMINE HYDROCHLORIDE:Mineral supplementation:0.69), (COLISTIN:Mineral supplementation:0.56), (CORTICOTROPIN:Mineral supplementation:1.38), (CRIZOTINIB:Mineral supplementation:1.16), (DACLATASVIR:Mineral supplementation:0.07), (DACLIZUMAB:Mineral supplementation:1.98), (DAPAGLIFLOZIN PROPANEDIOLMETFORMIN HYDROCHLORIDE:Mineral supplementation:0.47), (DEFERIPRONE:Mineral supplementation:0.83), (DEFEROXAMINE MESYLATE:Mineral supplementation:0.34), (DEXMETHYLPHENIDATE HYDROCHLORIDE:Mineral supplementation:0.01), (DIFLUNISAL:Mineral supplementation:0.32), (DIPHENHYDRAMINE HYDROCHLORIDELIDOCAINE HYDROCHLORIDENYSTATIN:Mineral supplementation:0.22), (DOLASETRON MESYLATE:Mineral supplementation:0.94), (DOXYLAMINE SUCCINATE:Mineral supplementation:0.64), (ECONAZOLE:Mineral supplementation:1.19), (EDOXABAN TOSILATE MONOHYDRATE:Mineral supplementation:0.21), (ELOTUZUMAB:Mineral supplementation:1.67), (ENALAPRIL MALEATEHYDROCHLOROTHIAZIDE:Mineral supplementation:0.41), (ENALAPRIL MALEATELERCANIDIPINE HYDROCHLORIDE:Mineral supplementation:0.34), (EPIRUBICIN:Mineral supplementation:1.27), (EPIRUBICIN HYDROCHLORIDE:Mineral supplementation:0.13), (ERIBULIN:Mineral supplementation:0.8), (ESTRADIOLLEVONORGESTREL:Mineral supplementation:0.11), (ETIMICIN SULFATE:Mineral supplementation:1.17), (ETOPOSIDE PHOSPHATE:Mineral supplementation:0.05), (ETRETINATE:Mineral supplementation:0.19), (EVENING PRIMROSE OIL:Mineral supplementation:0.1), (FERRIC HYDROXIDE:Mineral supplementation:0.83), (FERRIC OXYHYDROXIDE:Mineral supplementation:0.12), (FLUNARIZINE HYDROCHLORIDE:Mineral supplementation:0.02), (FLURAZEPAMFLURAZEPAM HYDROCHLORIDE:Mineral supplementation:0.47), (FLUTAMIDE:Mineral supplementation:0.19), (FLUVOXAMINE MALEATE:Mineral supplementation:1.25), (FORMOTEROL:Mineral supplementation:1.61), (FORMOTEROL FUMARATE:Mineral supplementation:1.91), (FORMOTEROL FUMARATE\MOMETASONE FUROATE:Mineral supplementation:1.37), (FROVATRIPTAN SUCCINATE:Mineral supplementation:0.33), (GEFITINIB:Mineral supplementation:0.63), (GIMERACILOTERACILTEGAFUR:Mineral supplementation:0.95), (GLIPIZIDEMETFORMIN HYDROCHLORIDE:Mineral supplementation:0.43), (GLYBURIDEMETFORMIN HYDROCHLORIDE:Mineral supplementation:0.25), (GLYCOPYRROLATEINDACATEROL MALEATE:Mineral supplementation:0.77), (HORSE CHESTNUT:Mineral supplementation:0.01), (HYDROCHLOROTHIAZIDERAMIPRIL:Mineral supplementation:0.15), (HYDROCODONE BITARTRATEIBUPROFEN:Mineral supplementation:0.97), (HYDROXYCHLOROQUINE DIPHOSPHATE:Mineral supplementation:1.45), (ICOSAPENT ETHYL:Mineral supplementation:0.74), (IMIQUIMOD:Mineral supplementation:0.38), (INSULIN DEGLUDEC:Mineral supplementation:1.27), (INSULIN PORK\INSULIN PURIFIED PORK:Mineral supplementation:0.53), (INTERFERON:Mineral supplementation:1.09), (INTERFERON ALFA-2A:Mineral supplementation:1.37), (IRINOTECAN HYDROCHLORIDE:Mineral supplementation:1.47), (ISOFLURANE:Mineral supplementation:0.25), (ITRACONAZOLE\SODIUM CHLORIDE:Mineral supplementation:0.04), (IVABRADINE HYDROCHLORIDE:Mineral supplementation:0.39), (IVACAFTOR:Mineral supplementation:0.62), (IVERMECTIN:Mineral supplementation:0.11), (LANREOTIDE:Mineral supplementation:0.57), (LANREOTIDE ACETATE:Mineral supplementation:0.3), (LAPATINIB DITOSYLATE:Mineral supplementation:0.93), (LENVATINIB:Mineral supplementation:0.59), (LEVODOPA:Mineral supplementation:0.03), (LEVOMILNACIPRAN HYDROCHLORIDE:Mineral supplementation:0.27), (LEVOTHYROXINE SODIUMPOTASSIUM IODIDE:Mineral supplementation:0.47), (LINSIDOMINE:Mineral supplementation:0.08), (LOMITAPIDE MESYLATE:Mineral supplementation:1.39), (LOMUSTINE:Mineral supplementation:0.22), (LUTETIUM LU-177:Mineral supplementation:0.37), (MAXACALCITOL:Mineral supplementation:0.13), (MEPOLIZUMAB:Mineral supplementation:1.12), (METAMIZOLE:Mineral supplementation:1.74), (METFORMIN HYDROCHLORIDEROSIGLITAZONE MALEATE:Mineral supplementation:0.14), (METFORMIN HYDROCHLORIDESAXAGLIPTIN:Mineral supplementation:0.19), (METHENAMINE MANDELATE:Mineral supplementation:0.39), (METHENOLONE ENANTHATE:Mineral supplementation:0.83), (METILDIGOXIN:Mineral supplementation:1.05), (METRELEPTIN:Mineral supplementation:0.22), (MICONAZOLE NITRATE:Mineral supplementation:0.81), (MIGLUSTAT:Mineral supplementation:0.44), (MILRINONE LACTATE:Mineral supplementation:0.15), (MOEXIPRIL HYDROCHLORIDE:Mineral supplementation:0.13), (MOXONIDINE:Mineral supplementation:1.13), (NALOXONE:Mineral supplementation:0.57), (NALOXONE HYDROCHLORIDETILIDINE HYDROCHLORIDE:Mineral supplementation:1.08), (NOREPINEPHRINE:Mineral supplementation:1.03), (NOREPINEPHRINE BITARTRATE:Mineral supplementation:1.27), (OCTREOTIDE:Mineral supplementation:0.41), (OSELTAMIVIR:Mineral supplementation:0.97), (OXYMORPHONE:Mineral supplementation:0.11), (PANCRELIPASEPANCRELIPASE AMYLASEPANCRELIPASE LIPASEPANCRELIPASE PROTEASE:Mineral supplementation:0.01), (PAPAYA:Mineral supplementation:0.07), (PATIROMER:Mineral supplementation:1.61), (PEGINTERFERON BETA-1A:Mineral supplementation:1.52), (PEGZEREPOETIN ALFA:Mineral supplementation:0.35), (PENICILLIN G:Mineral supplementation:1.4), (PENTOSTATIN:Mineral supplementation:0.41), (PHENYLEPHRINEPHENYLEPHRINE HYDROCHLORIDE:Mineral supplementation:0.28), (PILSICAINIDE HYDROCHLORIDE:Mineral supplementation:0.36), (POLYETHYLENE GLYCOL 4000:Mineral supplementation:0.87), (POLYMYXIN B SULFATETRIMETHOPRIM SULFATE:Mineral supplementation:0.27), (POTASSIUM CITRATETRISODIUM CITRATE DIHYDRATE:Mineral supplementation:0.11), (POTASSIUM PHOSPHATE, MONOBASIC\SODIUM PHOSPHATE, DIBASIC, ANHYDROUS\SODIUM PHOSPHATE, MONOBASIC, MONOHYDRATE:Mineral supplementation:0.05), (PRAMLINTIDE ACETATE:Mineral supplementation:0.3), (PRAMOXINE HYDROCHLORIDE:Mineral supplementation:0.13), (PRAZEPAM:Mineral supplementation:0.06), (PREDNISOLONE SODIUM PHOSPHATE:Mineral supplementation:0.98), (PRISTINAMYCIN:Mineral supplementation:0.14), (QUINACRINE:Mineral supplementation:0.46), (RALTEGRAVIR POTASSIUM:Mineral supplementation:0.86), (RAMUCIRUMAB:Mineral supplementation:0.94), (RETINOL:Mineral supplementation:1.66), (RILMENIDINE PHOSPHATE:Mineral supplementation:0.46), (RILPIVIRINE HYDROCHLORIDE:Mineral supplementation:0.03), (RUCAPARIB CAMSYLATE:Mineral supplementation:0.32), (RUFINAMIDE:Mineral supplementation:0.05), (SIBUTRAMINE HYDROCHLORIDE:Mineral supplementation:0.2), (SIPULEUCEL-T:Mineral supplementation:1.23), (SOFOSBUVIRVELPATASVIR:Mineral supplementation:0.47), (SOMATOSTATIN:Mineral supplementation:0.15), (SORBITOL:Mineral supplementation:0.52), (SUFENTANIL:Mineral supplementation:0.13), (SULBACTAM:Mineral supplementation:0.01), (TAFLUPROST:Mineral supplementation:0.36), (TEPRENONE:Mineral supplementation:0.54), (TESTOSTERONE UNDECANOATE:Mineral supplementation:0.97), (THROMBOPOIETIN:Mineral supplementation:0.03), (TIMOLOL MALEATETRAVOPROST:Mineral supplementation:0.29), (TRIHEXYPHENIDYL:Mineral supplementation:0.02), (TRIMIPRAMINE MALEATE:Mineral supplementation:0.13), (TRIPTORELIN PAMOATE:Mineral supplementation:0.75), (UBIQUINOL:Mineral supplementation:0.07), (UROKINASE:Mineral supplementation:0.19), (VALDECOXIB:Mineral supplementation:0.1), (VASOPRESSIN:Mineral supplementation:0.97), (VINORELBINEVINORELBINE TARTRATE:Mineral supplementation:0.67), (ZICONOTIDE ACETATE:Mineral supplementation:0.28), (ZILEUTON:Mineral supplementation:0.7), (ZIV-AFLIBERCEPT:Mineral supplementation:0.05), (BUMETANIDE:Mitral valve incompetence:0.25), (EVOLOCUMAB:Mitral valve incompetence:0.07), (ANASTROZOLE:Mitral valve prolapse:0.46), (DICLOFENAC SODIUM:Mitral valve prolapse:0.64), (EPINEPHRINE:Mitral valve prolapse:0.01), (EVOLOCUMAB:Mitral valve prolapse:0.56), (GLIPIZIDE:Mitral valve prolapse:0.11), (ILOPROST:Mitral valve prolapse:0.09), (INDOMETHACIN:Mitral valve prolapse:0.15), (METAXALONE:Mitral valve prolapse:0.47), (OLANZAPINE:Mitral valve prolapse:0.14), (OXCARBAZEPINE:Mitral valve prolapse:0.33), (OXYGEN:Mitral valve prolapse:0.21), (PAROXETINE:Mitral valve prolapse:0.3), (PROMETHAZINEPROMETHAZINE HYDROCHLORIDE:Mitral valve prolapse:0.07), (RANOLAZINE:Mitral valve prolapse:0.24), (TAMSULOSIN:Mitral valve prolapse:0.21), (TIZANIDINE HYDROCHLORIDE:Mitral valve prolapse:0.23), (TRIAMCINOLONE ACETONIDE:Mitral valve prolapse:0.06), (VITAMIN B:Mitral valve prolapse:0.04), (OXYGEN:Mitral valve replacement:0.14), (RIOCIGUAT:Mitral valve replacement:0.48), (DICLOFENAC:Mixed connective tissue disease:0.12), (IBRUTINIB:Mixed connective tissue disease:0.12), (BUMETANIDE:Monoclonal gammopathy:0.05), (DILTIAZEM HYDROCHLORIDE:Monoclonal gammopathy:0.06), (ENALAPRIL:Monoclonal gammopathy:0.17), (VERAPAMIL HYDROCHLORIDE:Monoclonal gammopathy:0.04), (ACETAMINOPHENBUTALBITALCAFFEINE:Mood altered:0.64), (ACETAMINOPHENOXYCODONEOXYCODONE HYDROCHLORIDE:Mood altered:0.01), (ACETAZOLAMIDE:Mood altered:0.09), (ALCOHOL:Mood altered:1.03), (ALENDRONIC ACID:Mood altered:0.11), (AMIODARONE HYDROCHLORIDE:Mood altered:0.11), (AZITHROMYCIN DIHYDRATE:Mood altered:0.09), (BENAZEPRIL HYDROCHLORIDE:Mood altered:0.12), (BUPRENORPHINE HYDROCHLORIDE:Mood altered:0.98), (CANDESARTAN:Mood altered:0.15), (CANDESARTAN CILEXETIL:Mood altered:0.28), (CANDESARTAN CILEXETILHYDROCHLOROTHIAZIDE:Mood altered:0.12), (CANNABIS SATIVA SUBSP. INDICA TOP:Mood altered:0.12), (CARVEDILOL PHOSPHATE:Mood altered:0.05), (CERTOLIZUMAB PEGOL:Mood altered:0.99), (CHOLESTYRAMINE:Mood altered:0.77), (CLINDAMYCINCLINDAMYCIN PHOSPHATE:Mood altered:0.65), (DASATINIB:Mood altered:0.31), (DESLORATADINE:Mood altered:0.11), (DOXAZOSIN MESYLATE:Mood altered:0.13), (DUTASTERIDE:Mood altered:0.48), (ELETRIPTAN HYDROBROMIDE:Mood altered:0.81), (EPINEPHRINE:Mood altered:0.31), (ETHAMBUTOL HYDROCHLORIDE:Mood altered:0.92), (ETORICOXIB:Mood altered:0.19), (EVOLOCUMAB:Mood altered:0.83), (FEBUXOSTAT:Mood altered:0.17), (FLECAINIDE:Mood altered:0.06), (FLUTICASONE:Mood altered:0.01), (FLUVOXAMINE MALEATE:Mood altered:0.03), (HYDRALAZINE HYDROCHLORIDE:Mood altered:0), (HYDROCHLOROTHIAZIDEVALSARTAN:Mood altered:0.37), (HYDROXYCHLOROQUINE DIPHOSPHATE:Mood altered:0.07), (HYOSCYAMINE:Mood altered:0.03), (IMATINIB MESYLATE:Mood altered:0.07), (IMIPRAMINE HYDROCHLORIDE:Mood altered:0.08), (ISOSORBIDE:Mood altered:0.17), (LABETALOLLABETALOL HYDROCHLORIDE:Mood altered:0.08), (LATANOPROST:Mood altered:0.29), (LEDIPASVIRSOFOSBUVIR:Mood altered:0.26), (METHENAMINE HIPPURATE:Mood altered:0), (MINOCYCLINEMINOCYCLINE HYDROCHLORIDE:Mood altered:0.21), (MOXIFLOXACIN HYDROCHLORIDE:Mood altered:0.81), (NEBIVOLOL:Mood altered:0.14), (NITROFURANTOIN:Mood altered:0.74), (PACLITAXEL:Mood altered:0.06), (PEGINTERFERON BETA-1A:Mood altered:0.14), (PENICILLIN:Mood altered:0.16), (PHENTERMINE HYDROCHLORIDE:Mood altered:0.07), (PONATINIB:Mood altered:0), (PROGESTERONE:Mood altered:0.29), (RABEPRAZOLE:Mood altered:0.06), (RALOXIFENE HYDROCHLORIDE:Mood altered:0.16), (RANOLAZINE:Mood altered:0.65), (SACUBITRILVALSARTAN:Mood altered:0.57), (SECUKINUMAB:Mood altered:0.41), (SULFAMETHOXAZOLE:Mood altered:0.03), (SUMATRIPTAN:Mood altered:0.38), (TELMISARTAN:Mood altered:0.65), (TERIFLUNOMIDE:Mood altered:0.74), (THALIDOMIDE:Mood altered:0.18), (TOCILIZUMAB:Mood altered:0.23), (TOLTERODINE TARTRATE:Mood altered:0.34), (UMECLIDINIUM BROMIDEVILANTEROL TRIFENATATE:Mood altered:0.14), (ZINC:Mood altered:0.17), (ZONISAMIDE:Mood altered:0.4), (ACETAMINOPHENOXYCODONEOXYCODONE HYDROCHLORIDE:Mood swings:0.1), (AMISULPRIDE:Mood swings:0.6), (ANASTROZOLE:Mood swings:0.34), (BISOPROLOL FUMARATE:Mood swings:0.19), (BUPRENORPHINE HYDROCHLORIDE:Mood swings:1.26), (CANAGLIFLOZINMETFORMIN HYDROCHLORIDE:Mood swings:0.25), (CANDESARTAN:Mood swings:0.32), (CANDESARTAN CILEXETIL:Mood swings:0.57), (CERTOLIZUMAB PEGOL:Mood swings:0.79), (CHOLESTYRAMINE:Mood swings:0.48), (CLOBAZAM:Mood swings:0.35), (CODEINE:Mood swings:0.04), (CYPROHEPTADINE:Mood swings:0.08), (DACLIZUMAB:Mood swings:0.11), (DALFAMPRIDINE:Mood swings:0.82), (DAPAGLIFLOZIN PROPANEDIOL:Mood swings:0.44), (DASATINIB:Mood swings:0.28), (DESLORATADINE:Mood swings:0.13), (DOCUSATE:Mood swings:0.11), (DOFETILIDE:Mood swings:0.15), (DOXEPIN HYDROCHLORIDE:Mood swings:0.05), (DRONABINOL:Mood swings:0.31), (DUTASTERIDE:Mood swings:0.36), (EMPAGLIFLOZINLINAGLIPTIN:Mood swings:0.54), (ENOXAPARIN:Mood swings:0.57), (ESOMEPRAZOLE:Mood swings:0.17), (EVOLOCUMAB:Mood swings:1.3), (FLURAZEPAMFLURAZEPAM HYDROCHLORIDE:Mood swings:0.05), (FLUTICASONE:Mood swings:0.21), (FLUTICASONE FUROATEVILANTEROL TRIFENATATE:Mood swings:0.02), (INDAPAMIDE:Mood swings:0.03), (LABETALOLLABETALOL HYDROCHLORIDE:Mood swings:0.29), (LEDIPASVIRSOFOSBUVIR:Mood swings:0.16), (LEVALBUTEROL HYDROCHLORIDE:Mood swings:0.06), (LINAGLIPTIN:Mood swings:0.2), (METAXALONE:Mood swings:1.23), (METHYLPREDNISOLONE ACETATE:Mood swings:0.28), (METHYLPREDNISOLONE SODIUM SUCCINATE:Mood swings:0.61), (METRONIDAZOLEMETRONIDAZOLE HYDROCHLORIDE:Mood swings:0.13), (MOXIFLOXACIN HYDROCHLORIDE:Mood swings:0.71), (NEBIVOLOL:Mood swings:0.26), (NILOTINIB:Mood swings:0.03), (OSPEMIFENE:Mood swings:0.64), (OXYMORPHONE:Mood swings:0.07), (PEGINTERFERON BETA-1A:Mood swings:0.1), (PHENOBARBITAL:Mood swings:0.62), (RABEPRAZOLE:Mood swings:0.09), (RABEPRAZOLE SODIUM:Mood swings:0.27), (RIVASTIGMINE:Mood swings:0.23), (SECUKINUMAB:Mood swings:0.4), (SOLIFENACIN SUCCINATE:Mood swings:0.08), (SUMATRIPTAN:Mood swings:0.73), (SUVOREXANT:Mood swings:0.02), (TELMISARTAN:Mood swings:0.28), (TETRACYCLINE:Mood swings:0.14), (TIMOLOL:Mood swings:0.12), (TOCILIZUMAB:Mood swings:0.1), (TOLTERODINE TARTRATE:Mood swings:0.33), (TRIAMTERENE:Mood swings:0.38), (UMECLIDINIUM BROMIDEVILANTEROL TRIFENATATE:Mood swings:0.19), (ARMODAFINIL:Motion sickness:0.03), (BUSPIRONE HYDROCHLORIDE:Motion sickness:0.04), (CITALOPRAM HYDROBROMIDE:Motion sickness:0.49), (IBRUTINIB:Motion sickness:0.03), (LANSOPRAZOLE:Motion sickness:0.11), (LOVASTATIN:Motion sickness:0.43), (MELOXICAM:Motion sickness:0.12), (METHYLPREDNISOLONE SODIUM SUCCINATE:Motion sickness:0.02), (OXCARBAZEPINE:Motion sickness:0.26), (TERIFLUNOMIDE:Motion sickness:0.38), (AMIODARONE:Mouth ulceration:0.4), (ANASTROZOLE:Mouth ulceration:1.13), (BENDAMUSTINE HYDROCHLORIDE:Mouth ulceration:0.18), (CALCITRIOL:Mouth ulceration:0.32), (CARFILZOMIB:Mouth ulceration:0.07), (CLINDAMYCINCLINDAMYCIN PHOSPHATE:Mouth ulceration:1.07), (DASATINIB:Mouth ulceration:0.23), (DEXLANSOPRAZOLE:Mouth ulceration:0.73), (DULOXETINE:Mouth ulceration:0.05), (EVOLOCUMAB:Mouth ulceration:0.34), (HYDROCODONE:Mouth ulceration:0.17), (HYDROXYCHLOROQUINE DIPHOSPHATE:Mouth ulceration:0.07), (IRBESARTAN:Mouth ulceration:0.02), (IRINOTECAN HYDROCHLORIDE:Mouth ulceration:0.04), (ISOSORBIDE MONONITRATE:Mouth ulceration:0.15), (LEDIPASVIRSOFOSBUVIR:Mouth ulceration:0.27), (LEUCOVORIN CALCIUM:Mouth ulceration:0.62), (METRONIDAZOLE:Mouth ulceration:1.65), (NILOTINIB:Mouth ulceration:0.77), (POLYETHYLENE GLYCOLS:Mouth ulceration:0.58), (PRAVASTATIN:Mouth ulceration:0.35), (PRAVASTATIN SODIUM:Mouth ulceration:0.55), (PYRAZINAMIDE:Mouth ulceration:0.65), (SULFAMETHOXAZOLE:Mouth ulceration:0.07), (TAMSULOSIN:Mouth ulceration:0.66), (TORSEMIDE:Mouth ulceration:0.15), (VALGANCICLOVIR HYDROCHLORIDE:Mouth ulceration:0.42), (VANCOMYCIN HYDROCHLORIDE:Mouth ulceration:0.21), (VERAPAMIL HYDROCHLORIDE:Mouth ulceration:0.39), (ATENOLOL:Movement disorder:0.09), (CLOBAZAM:Movement disorder:0.17), (CYCLOBENZAPRINE HYDROCHLORIDE:Movement disorder:0), (DEXLANSOPRAZOLE:Movement disorder:0.25), (DICLOFENAC:Movement disorder:0.01), (DIGOXIN:Movement disorder:0.35), (DILTIAZEM:Movement disorder:0.02), (ENALAPRIL:Movement disorder:0.31), (EVOLOCUMAB:Movement disorder:0.2), (HYDROXYZINE PAMOATE:Movement disorder:0.04), (LISDEXAMFETAMINE DIMESYLATE:Movement disorder:0.14), (NAPROXEN:Movement disorder:0.64), (PAROXETINE:Movement disorder:0.38), (SACUBITRILVALSARTAN:Movement disorder:0.29), (VARENICLINE TARTRATE:Movement disorder:0.89), (ACETAMINOPHENHYDROCODONE:Mucosal inflammation:0.06), (ACETAMINOPHENPROPOXYPHENE HYDROCHLORIDE:Mucosal inflammation:0.09), (ALENDRONIC ACID:Mucosal inflammation:0.16), (AMPICILLIN:Mucosal inflammation:0.67), (ASCORBIC ACID:Mucosal inflammation:0.51), (BENAZEPRILBENAZEPRIL HYDROCHLORIDE:Mucosal inflammation:0.06), (BISACODYL OR DOCUSATE SODIUM:Mucosal inflammation:0.55), (BUDESONIDEFORMOTEROL FUMARATE:Mucosal inflammation:0.09), (BUPRENORPHINE HYDROCHLORIDE:Mucosal inflammation:0.03), (BUPROPION:Mucosal inflammation:0.44), (CALCITONIN:Mucosal inflammation:0.04), (CALCITONIN SALMON:Mucosal inflammation:0.47), (CALCITRIOL:Mucosal inflammation:1.18), (CALCIUM ACETATE:Mucosal inflammation:0.22), (CANAKINUMAB:Mucosal inflammation:0.2), (CANDESARTAN CILEXETIL:Mucosal inflammation:0.9), (CANDESARTAN CILEXETILHYDROCHLOROTHIAZIDE:Mucosal inflammation:1.04), (CARFILZOMIB:Mucosal inflammation:0.97), (CHLORAMBUCIL:Mucosal inflammation:0.72), (CHOLESTYRAMINE:Mucosal inflammation:1.17), (CLADRIBINE:Mucosal inflammation:0.1), (CLOZAPINE:Mucosal inflammation:0.25), (CYCLOBENZAPRINE:Mucosal inflammation:0.34), (DABRAFENIB:Mucosal inflammation:0.8), (DANAZOL:Mucosal inflammation:0.1), (DAUNORUBICIN HYDROCHLORIDE:Mucosal inflammation:0.04), (DEXAMETHASONE SODIUM PHOSPHATE:Mucosal inflammation:0.22), (DILTIAZEM HYDROCHLORIDE:Mucosal inflammation:1.08), (DIVALPROEX SODIUM:Mucosal inflammation:0.1), (DOFETILIDE:Mucosal inflammation:0.06), (DUTASTERIDE:Mucosal inflammation:0.45), (ECONAZOLE:Mucosal inflammation:0.4), (ELOTUZUMAB:Mucosal inflammation:0.4), (EZETIMIBESIMVASTATIN:Mucosal inflammation:0.3), (FESOTERODINE FUMARATE:Mucosal inflammation:0.07), (FLECAINIDE:Mucosal inflammation:0.68), (FLUOXETINEFLUOXETINE HYDROCHLORIDE:Mucosal inflammation:0.15), (FLUTICASONE FUROATE:Mucosal inflammation:0.12), (FORMOTEROL FUMARATE DIHYDRATE\MOMETASONE FUROATE:Mucosal inflammation:0.17), (FORMOTEROL FUMARATE\MOMETASONE FUROATE:Mucosal inflammation:0.02), (FOSFOMYCIN:Mucosal inflammation:0.24), (GEFITINIB:Mucosal inflammation:0.16), (GIMERACILOTERACILTEGAFUR:Mucosal inflammation:0.17), (GLYCOPYRRONIUMINDACATEROL:Mucosal inflammation:0.36), (IBRITUMOMAB TIUXETAN:Mucosal inflammation:0.65), (IMATINIB MESYLATE:Mucosal inflammation:1.6), (ISONIAZID:Mucosal inflammation:0.43), (ISOSORBIDE:Mucosal inflammation:0.11), (ISOSORBIDE MONONITRATE:Mucosal inflammation:0.53), (IXAZOMIB:Mucosal inflammation:0.16), (LAPATINIB DITOSYLATE:Mucosal inflammation:0.27), (LEDIPASVIRSOFOSBUVIR:Mucosal inflammation:0.38), (LERCANIDIPINE HYDROCHLORIDE:Mucosal inflammation:0.94), (LEVALBUTEROL HYDROCHLORIDE:Mucosal inflammation:0.46), (LINAGLIPTIN:Mucosal inflammation:0.43), (METHYLPREDNISOLONE ACETATE:Mucosal inflammation:0.37), (OCTREOTIDE ACETATE:Mucosal inflammation:0.07), (OLODATEROL HYDROCHLORIDETIOTROPIUM BROMIDE MONOHYDRATE:Mucosal inflammation:0.12), (OMEGA-3-ACID ETHYL ESTERS:Mucosal inflammation:0.11), (OXCARBAZEPINE:Mucosal inflammation:0.5), (PAROXETINE:Mucosal inflammation:0.35), (PAROXETINEPAROXETINE HYDROCHLORIDE:Mucosal inflammation:0.02), (PEMETREXED DISODIUM:Mucosal inflammation:0.69), (PENICILLIN:Mucosal inflammation:0.61), (PENTOSTATIN:Mucosal inflammation:0.51), (PREDNISOLONE SODIUM PHOSPHATE:Mucosal inflammation:0.3), (PRIMIDONE:Mucosal inflammation:0.06), (QUETIAPINE:Mucosal inflammation:0.19), (RABEPRAZOLE:Mucosal inflammation:0.36), (RAMUCIRUMAB:Mucosal inflammation:0.35), (ROSUVASTATIN:Mucosal inflammation:0.03), (SACUBITRILVALSARTAN:Mucosal inflammation:0.3), (SECUKINUMAB:Mucosal inflammation:0.66), (SITAGLIPTIN:Mucosal inflammation:0.12), (SULFAMETHOXAZOLE:Mucosal inflammation:0.88), (TERBINAFINE HYDROCHLORIDE:Mucosal inflammation:0.12), (TIGECYCLINE:Mucosal inflammation:0.33), (TIOTROPIUM:Mucosal inflammation:0.39), (TIZANIDINE:Mucosal inflammation:0.08), (TIZANIDINE HYDROCHLORIDE:Mucosal inflammation:0.02), (TOCILIZUMAB:Mucosal inflammation:0.25), (TOFACITINIB CITRATE:Mucosal inflammation:0.29), (TRAMETINIB:Mucosal inflammation:0.67), (VALGANCICLOVIR HYDROCHLORIDE:Mucosal inflammation:0.37), (VINORELBINE TARTRATE:Mucosal inflammation:0.26), (ZIV-AFLIBERCEPT:Mucosal inflammation:0.26), (ABOBOTULINUMTOXINA:Multiple allergies:0.44), (ACARBOSE:Multiple allergies:0.39), (ACETAMINOPHENOXYCODONE HYDROCHLORIDEOXYCODONE TEREPHTHALATE:Multiple allergies:1.33), (ACREMONIUM STRICTUM:Multiple allergies:0.77), (ADO-TRASTUZUMAB EMTANSINE:Multiple allergies:0.14), (AGALSIDASE BETA:Multiple allergies:0.45), (ALBUTEROLIPRATROPIUM:Multiple allergies:0.23), (ALENDRONIC ACID:Multiple allergies:0.49), (ALFACALCIDOL:Multiple allergies:0.12), (RETROFLEXUS POLLENCHENOPODIUM ALBUM POLLENSTRUMARIUM POLLEN:Multiple allergies:1.04), (AMINO ACIDSDEXTROSEELECTROLYTES NOS:Multiple allergies:0.18), (AMLODIPINE BESYLATEATORVASTATIN CALCIUM:Multiple allergies:0.25), (AMMONIUM CHLORIDEDEXTROMETHORPHAN\SODIUM CITRATE:Multiple allergies:0.26), (AMPICILLIN SODIUMSULBACTAM SODIUM:Multiple allergies:0.12), (ANTHOXANTHUM ODORATUM POLLENDACTYLIS GLOMERATA POLLENLOLIUM PERENNE POLLENPHLEUM PRATENSE POLLENPOA PRATENSIS POLLEN:Multiple allergies:0.16), (ARIPIPRAZOLE LAUROXIL:Multiple allergies:0.26), (BETAMETHASONECALCIPOTRIENE:Multiple allergies:0.33), (NIGRA POLLENCARYA OVATA POLLENAMERICANA POLLEN POPULUS DELTOIDES POLLENQUERCUS RUBRA POLLENULMUS AMERICANA POLLEN:Multiple allergies:1.04), (BROMAZEPAM:Multiple allergies:0.32), (BUPRENORPHINE HYDROCHLORIDE:Multiple allergies:1.08), (BUPRENORPHINENALOXONE:Multiple allergies:0.13), (BUTYLSCOPOLAMINE BROMIDE:Multiple allergies:0.11), (CALCIUM CARBONATEERGOCALCIFEROLRETINOL:Multiple allergies:0.09), (CANDESARTAN CILEXETILHYDROCHLOROTHIAZIDE:Multiple allergies:1.3), (CARVEDILOL PHOSPHATE:Multiple allergies:2.11), (CARYA OVATA POLLEN:Multiple allergies:1.04), (CEFPROZIL:Multiple allergies:0.15), (CEFTRIAXONE SODIUM:Multiple allergies:0.85), (CEVIMELINE HYDROCHLORIDE:Multiple allergies:0.38), (CHLORPROMAZINE:Multiple allergies:0.08), (CILASTATIN SODIUMIMIPENEM:Multiple allergies:0.08), (CINACALCET HYDROCHLORIDE:Multiple allergies:0.44), (CLOBAZAM:Multiple allergies:0.11), (CLOMIPRAMINE HYDROCHLORIDE:Multiple allergies:0.01), (CODEINE PHOSPHATE:Multiple allergies:0.06), (CURCUMIN:Multiple allergies:0.05), (CYPROHEPTADINE HYDROCHLORIDE:Multiple allergies:0.13), (DACLIZUMAB:Multiple allergies:1.36), (DANAZOL:Multiple allergies:0.42), (DASABUVIROMBITASVIRPARITAPREVIRRITONAVIR:Multiple allergies:0.11), (DASATINIB:Multiple allergies:1.78), (DEFLAZACORT:Multiple allergies:0.53), (DICLOXACILLIN:Multiple allergies:0.04), (DIPYRONE:Multiple allergies:0.25), (DOXEPIN HYDROCHLORIDE:Multiple allergies:0.94), (EDOXABAN TOSYLATE:Multiple allergies:0.84), (ESTRADIOLLEVONORGESTREL:Multiple allergies:0.02), (ESTRIOL:Multiple allergies:0.04), (FENOTEROL HYDROBROMIDE:Multiple allergies:0.12), (FERRIC POLYSACCHARIDE COMPLEX:Multiple allergies:0.04), (FINGOLIMOD HYDROCHLORIDE:Multiple allergies:0.48), (FLURAZEPAMFLURAZEPAM HYDROCHLORIDE:Multiple allergies:0.05), (FLUTICASONE FUROATESALMETEROL XINAFOATE:Multiple allergies:1.42), (FORMOTEROL FUMARATE\MOMETASONE FUROATE:Multiple allergies:1.39), (FUSIDIC ACID:Multiple allergies:0.43), (GLYCOPYRROLATE:Multiple allergies:0.74), (GLYCOPYRROLATEINDACATEROL MALEATE:Multiple allergies:0.16), (GLYCOPYRRONIUM:Multiple allergies:0.69), (GLYCOPYRRONIUMINDACATEROL:Multiple allergies:0.71), (HYDROCODONE BITARTRATEIBUPROFEN:Multiple allergies:0.77), (HYDROXYCHLOROQUINE DIPHOSPHATE:Multiple allergies:0.57), (HYOSCYAMINE SULFATEMETHENAMINEMETHYLENE BLUEPHENYL SALICYLATE\SODIUM PHOSPHATE, MONOBASIC, MONOHYDRATE:Multiple allergies:0.22), (ICOSAPENT ETHYL:Multiple allergies:0.07), (ILOPROST:Multiple allergies:0.7), (IMIPRAMINE HYDROCHLORIDE:Multiple allergies:1.47), (INDACATEROL:Multiple allergies:0.52), (INDAPAMIDEPERINDOPRIL:Multiple allergies:0.36), (INFLIXIMAB-DYYB:Multiple allergies:0.23), (INSULIN GLULISINE:Multiple allergies:0.47), (INTERFERON GAMMA-1B:Multiple allergies:0.11), (ISOPROPYL ALCOHOLLEUPROLIDE ACETATENORETHINDRONE ACETATE:Multiple allergies:0.4), (IXEKIZUMAB:Multiple allergies:0.49), (LAPATINIB DITOSYLATE:Multiple allergies:0), (LEDIPASVIRSOFOSBUVIR:Multiple allergies:0.77), (LERCANIDIPINE HYDROCHLORIDE:Multiple allergies:0.06), (LOMITAPIDE MESYLATE:Multiple allergies:0.93), (MEDROXYPROGESTERONE:Multiple allergies:0.5), (MEFENAMIC ACID:Multiple allergies:0.38), (MOEXIPRIL HYDROCHLORIDE:Multiple allergies:0.28), (MOXIFLOXACIN:Multiple allergies:0.61), (NALOXONE HYDROCHLORIDEOXYCODONE HYDROCHLORIDE:Multiple allergies:0.69), (NATEGLINIDE:Multiple allergies:0.64), (NEPAFENAC:Multiple allergies:0.11), (NILOTINIB:Multiple allergies:0.96), (OMEPRAZOLE SODIUM:Multiple allergies:0.08), (ORPHENADRINE:Multiple allergies:0.18), (PANTOPRAZOLE MAGNESIUM:Multiple allergies:1.93), (PAPAYA:Multiple allergies:0.07), (PATIROMER:Multiple allergies:0.32), (PAZOPANIB HYDROCHLORIDE:Multiple allergies:0.27), (PENICILLIN V:Multiple allergies:0.18), (PHENYTOIN SODIUM:Multiple allergies:0.21), (PIRFENIDONE:Multiple allergies:0.27), (PROTRIPTYLINE HYDROCHLORIDE:Multiple allergies:0.87), (RIVASTIGMINE:Multiple allergies:0.29), (SCOPOLAMINE:Multiple allergies:1.2), (SELEGILINE:Multiple allergies:0.01), (SILVER SULFADIAZINE:Multiple allergies:0.07), (SUNITINIB MALATE:Multiple allergies:0.62), (TASIMELTEON:Multiple allergies:0.1), (TERBUTALINE SULFATE:Multiple allergies:2.09), (TESTOSTERONE UNDECANOATE:Multiple allergies:0.32), (TETRACYCLINE:Multiple allergies:2.02), (TOLTERODINE:Multiple allergies:0.36), (VORICONAZOLE:Multiple allergies:0.13), (ZOPICLONE:Multiple allergies:1.22), (.ALPHA.-TOCOPHEROL ACETATE, D-ASCORBIC ACIDCALCIUM PANTOTHENATEFOLIC ACIDMANGANESENIACINAMIDEPYRIDOXINE\RIBOFLAVINTHIAMINEVITAMIN A\ZINC OXIDE:Multiple sclerosis:0.33), (.ALPHA.1-PROTEINASE INHIBITOR HUMAN:Multiple sclerosis:1.2), (.BETA.-CAROTENE:Multiple sclerosis:0.42), (ABACAVIR SULFATEDOLUTEGRAVIR SODIUMLAMIVUDINE:Multiple sclerosis:0.18), (ABIRATERONE ACETATE:Multiple sclerosis:0.9), (ACAMPROSATE:Multiple sclerosis:0.62), (ACEBUTOLOL HYDROCHLORIDE:Multiple sclerosis:1.26), (ACETAMINOPHEN\ASPIRIN:Multiple sclerosis:0.2), (ACETAMINOPHEN\CAFFEINEPROPOXYPHENE:Multiple sclerosis:0.18), (ACETAMINOPHENCHLORPHENIRAMINE MALEATEDEXTROMETHORPHAN HYDROBROMIDEPHENYLEPHRINE HYDROCHLORIDE OR ACETAMINOPHENDEXTROMETHORPHAN HYDROBROMIDEDOXYLAMINE SUCCINATEPHENYLEPHRINE HYDROCHLORIDE OR HOMEOPATHICS:Multiple sclerosis:0.11), (ACETAMINOPHENDICHLORALPHENAZONEISOMETHEPTENE HYDROCHLORIDE:Multiple sclerosis:0.43), (ACETYLCYSTEINEBENZALKONIUM CHLORIDETUAMINOHEPTANE SULFATE:Multiple sclerosis:0.22), (ACRIVASTINEPSEUDOEPHEDRINE HYDROCHLORIDE:Multiple sclerosis:0.33), (ADAPALENEBENZOYL PEROXIDE:Multiple sclerosis:0.31), (ADENOSINE:Multiple sclerosis:0.8), (ADO-TRASTUZUMAB EMTANSINE:Multiple sclerosis:0.86), (AJMALINE:Multiple sclerosis:0.72), (ALCOHOLALUMINUM CHLORIDE:Multiple sclerosis:0.85), (ALGLUCOSIDASE ALFA:Multiple sclerosis:0.18), (ALISKIREN HEMIFUMARATEHYDROCHLOROTHIAZIDE:Multiple sclerosis:0.99), (ALISKIREN HEMIFUMARATEVALSARTAN:Multiple sclerosis:0.66), (ALISKIRENHYDROCHLOROTHIAZIDE:Multiple sclerosis:0.15), (ALLANTOINBENZOCAINECAMPHOR (NATURAL)DIMETHICONEMENTHOL:Multiple sclerosis:0.99), (ALOGLIPTIN BENZOATE:Multiple sclerosis:0.41), (AMILORIDE HYDROCHLORIDEFUROSEMIDE:Multiple sclerosis:0.97), (AMINO ACIDSDEXTROSEELECTROLYTES NOS:Multiple sclerosis:1.71), (AMINOBENZOIC ACIDCALCIUM PANTOTHENATECYSTINEKERATINTHIAMINE MONONITRATE:Multiple sclerosis:0.72), (AMINOSALICYLIC ACID:Multiple sclerosis:0.12), (AMITRIPTYLINEPERPHENAZINE:Multiple sclerosis:0.2), (AMLODIPINE MALEATE:Multiple sclerosis:0.08), (AMOBARBITAL SODIUM:Multiple sclerosis:0.1), (AMPHETAMINE ASPARTATE MONOHYDRATEAMPHETAMINE SULFATEDEXTROAMPHETAMINE SACCHARATEDEXTROAMPHETAMINE SULFATE:Multiple sclerosis:0.17), (AMPHETAMINE ASPARTATEDEXTROAMPHETAMINE SACCHARATE:Multiple sclerosis:0.01), (ARCTOSTAPHYLOS UVA-URSI LEAFURTICA DIOICA:Multiple sclerosis:0.16), (ARIPIPRAZOLE LAUROXIL:Multiple sclerosis:1.43), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE HYDROCHLORIDE:Multiple sclerosis:0.02), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE MONONITRATE:Multiple sclerosis:0.43), (ASENAPINE:Multiple sclerosis:0.02), (ASPIRIN\CAFFEINESALICYLAMIDE:Multiple sclerosis:0.74), (ASPIRINCLOPIDOGREL:Multiple sclerosis:0.12), (ATOVAQUONEPROGUANIL HYDROCHLORIDE:Multiple sclerosis:0.36), (ATROPINE SULFATE:Multiple sclerosis:0.99), (AVOBENZONEHOMOSALATEOCTISALATE:Multiple sclerosis:0.03), (AVOCADO OILSOYBEAN OIL:Multiple sclerosis:0.25), (AZELNIDIPINE:Multiple sclerosis:0.42), (AZITHROMYCIN MONOHYDRATE:Multiple sclerosis:1.91), (AZITHROMYCIN OR METHOTREXATE SODIUM:Multiple sclerosis:1.08), (BACITRACIN ZINC:Multiple sclerosis:0.48), (BELATACEPT:Multiple sclerosis:0.76), (BENDAMUSTINE HYDROCHLORIDE:Multiple sclerosis:0.07), (BERAPROST SODIUM:Multiple sclerosis:0.68), (BETA GLUCAN:Multiple sclerosis:0.21), (BETAMETHASONE ACETATEBETAMETHASONE SODIUM PHOSPHATE:Multiple sclerosis:0.94), (BETAMETHASONE DIPROPIONATEBETAMETHASONE SODIUM PHOSPHATE:Multiple sclerosis:0.58), (BETAMETHASONE DIPROPIONATECALCIPOTRIENE:Multiple sclerosis:1.33), (BETAMETHASONE DIPROPIONATECALCIPOTRIENE HYDRATE:Multiple sclerosis:0.73), (BETAMETHASONE SODIUM PHOSPHATE:Multiple sclerosis:1.01), (BETAMETHASONECALCIPOTRIENE:Multiple sclerosis:1.73), (BISMUTH SUBCITRATE POTASSIUMMETRONIDAZOLETETRACYCLINE HYDROCHLORIDE:Multiple sclerosis:0.44), (BLINATUMOMAB:Multiple sclerosis:0.43), (BLONANSERIN:Multiple sclerosis:0.61), (BOCEPREVIR:Multiple sclerosis:0.46), (BORIC ACID:Multiple sclerosis:0.68), (BORON:Multiple sclerosis:0.78), (BRIMONIDINE TARTRATEDORZOLAMIDE HYDROCHLORIDETIMOLOL MALEATE:Multiple sclerosis:0.17), (BROMPHENIRAMINE MALEATEDEXTROMETHORPHAN HYDROBROMIDEPSEUDOEPHEDRINE HYDROCHLORIDE:Multiple sclerosis:0.04), (BUDESONIDEINDACATEROL MALEATE:Multiple sclerosis:0.05), (BUPRENORPHINENALOXONE:Multiple sclerosis:0.76), (BUTABARBITAL:Multiple sclerosis:0.48), (BUTYLSCOPOLAMINE:Multiple sclerosis:0.13), (CALCIUM CARBIMIDE:Multiple sclerosis:0.42), (CALCIUM CARBONATEETIDRONATE DISODIUM:Multiple sclerosis:1.15), (CALCIUM CARBONATEMAGNESIUM CARBONATEMAGNESIUM TRISILICATE:Multiple sclerosis:0.59), (CALCIUM PHOSPHATE:Multiple sclerosis:0.02), (CALCIUMERGOCALCIFEROL:Multiple sclerosis:0.06), (CANAKINUMAB:Multiple sclerosis:0.94), (CARBOMER 934GLYCERINMETHYLPARABENPROPYLPARABEN\SODIUM HYDROXIDE:Multiple sclerosis:0.23), (CARFILZOMIB:Multiple sclerosis:1.7), (CARTEOLOL:Multiple sclerosis:0.08), (CASPOFUNGIN:Multiple sclerosis:0.22), (CEFOTAXIMECEFOTAXIME SODIUM:Multiple sclerosis:0.13), (CEFPROZIL:Multiple sclerosis:1.56), (CERIVASTATIN SODIUM:Multiple sclerosis:1.22), (CHAMOMILE:Multiple sclerosis:0.52), (CHLOPHEDIANOL HYDROCHLORIDEDEXBROMPHENIRAMINE MALEATEPHENYLEPHRINE HYDROCHLORIDE:Multiple sclerosis:0.1), (CHLORAL HYDRATE:Multiple sclerosis:0.12), (CHLORAMBUCIL:Multiple sclerosis:0.53), (CHLORDIAZEPOXIDECLIDINIUM:Multiple sclerosis:0.64), (CHLORMEZANONE:Multiple sclerosis:0.06), (CHLOROQUINECHLOROQUINE PHOSPHATE:Multiple sclerosis:0.21), (CHLORPROTHIXENE:Multiple sclerosis:0.49), (CHOLECALCIFEROLGENISTEINZINC GLYCINATE:Multiple sclerosis:0.43), (CIPROFLOXACIN HYDROCHLORIDEHYDROCORTISONE:Multiple sclerosis:0.05), (CISAPRIDE:Multiple sclerosis:1.03), (CLIDINIUM BROMIDE:Multiple sclerosis:0.03), (CLIKSTAR:Multiple sclerosis:1.44), (CLOMETHIAZOLE:Multiple sclerosis:0.14), (CLONIXINCYCLOBENZAPRINE:Multiple sclerosis:0.37), (TETANI TOXOID ANTIGEN (FORMALDEHYDE INACTIVATED):Multiple sclerosis:0.73), (CLOSTRIDIUM TETANICLOSTRIDIUM TETANI TOXOID ANTIGEN (FORMALDEHYDE INACTIVATED)DIPHTHERIAECORYNEBACTERIUM DIPHTHERIAE TOXOID ANTIGEN (FORMALDEHYDE INACTIVATED):Multiple sclerosis:0.05), (CLOTRIMAZOLEHYDROCORTISONE:Multiple sclerosis:0.13), (COBICISTATELVITEGRAVIREMTRICITABINETENOFOVIR ALAFENAMIDE FUMARATE:Multiple sclerosis:0.41), (CODEINE PHOSPHATEGUAIFENESINPSEUDOEPHEDRINE HYDROCHLORIDE:Multiple sclerosis:0.15), (COLISTIMETHATE SODIUM:Multiple sclerosis:0.12), (CROTALIDAE POLYVALENT IMMUNE FAB (OVINE):Multiple sclerosis:0.03), (CUMIN:Multiple sclerosis:0.07), (CYSTEINE:Multiple sclerosis:0.05), (DABIGATRAN:Multiple sclerosis:1.15), (DACLATASVIR:Multiple sclerosis:0.13), (DARATUMUMAB:Multiple sclerosis:0.18), (DEGARELIX:Multiple sclerosis:0.02), (DEUTETRABENAZINE:Multiple sclerosis:0.38), (DIBUCAINE:Multiple sclerosis:0.16), (DICHLORPHENAMIDE:Multiple sclerosis:0.74), (DIETARY SUPPLEMENTFLAVOCOXID:Multiple sclerosis:0.31), (DIETARY SUPPLEMENTRESVERATROL:Multiple sclerosis:1.03), (DIHYDRALAZINE SULFATE:Multiple sclerosis:0.39), (DIHYDROCODEINE PHOSPHATE:Multiple sclerosis:0.36), (DIMETHYL SULFONEGLUCOSAMINE SULFATE:Multiple sclerosis:0.14), (DIMETHYL SULFOXIDE:Multiple sclerosis:0.74), (DISOPYRAMIDE:Multiple sclerosis:0.09), (DISOPYRAMIDE PHOSPHATE:Multiple sclerosis:0.63), (DOBUTAMINE HYDROCHLORIDE:Multiple sclerosis:0.31), (DOCONEXENT ICOSAPENT FISH OIL\OMEGA-3 FATTY ACIDS:Multiple sclerosis:0.05), (DOFETILIDE:Multiple sclerosis:2.19), (DOLASETRON MESYLATE:Multiple sclerosis:1.26), (DOXORUBICIN HYDROCHLORIDE:Multiple sclerosis:0.03), (DOXYLAMINEPYRIDOXINE:Multiple sclerosis:0.34), (DROPERIDOL:Multiple sclerosis:0.43), (ECALLANTIDE:Multiple sclerosis:0.52), (ELOTUZUMAB:Multiple sclerosis:0.83), (EMPAGLIFLOZINMETFORMIN HYDROCHLORIDE:Multiple sclerosis:0.2), (EMTRICITABINERILPIVIRINE HYDROCHLORIDETENOFOVIR DISOPROXIL FUMARATE:Multiple sclerosis:0.45), (EMTRICITABINETENOFOVIR ALAFENAMIDE FUMARATE:Multiple sclerosis:0.1), (ENALAPRILHYDROCHLOROTHIAZIDE:Multiple sclerosis:0.2), (ENTACAPONE:Multiple sclerosis:0.57), (ERGOLOID MESYLATES:Multiple sclerosis:0.8), (ESMOLOL HYDROCHLORIDE:Multiple sclerosis:0.18), (ESSENTIAL OILS:Multiple sclerosis:0.32), (ESTRADIOL BENZOATE:Multiple sclerosis:1.59), (ESTRADIOLESTRIOL:Multiple sclerosis:0.31), (ESTRADIOLLEVONORGESTREL:Multiple sclerosis:1.01), (ETILEFRINE HYDROCHLORIDE:Multiple sclerosis:0.33), (ETRETINATE:Multiple sclerosis:0.51), (EUCALYPTOLMENTHOLMETHYL SALICYLATETHYMOL:Multiple sclerosis:0.14), (FELBAMATE:Multiple sclerosis:0.64), (FERRIC CARBOXYMALTOSE:Multiple sclerosis:0.16), (FERRIC POLYSACCHARIDE COMPLEX:Multiple sclerosis:1.14), (FERRITIN:Multiple sclerosis:0.03), (FERROUS FUMARATEFOLIC ACIDVITAMIN B COMPLEX:Multiple sclerosis:0.09), (FLIBANSERIN:Multiple sclerosis:0.05), (FLUNARIZINE HYDROCHLORIDE:Multiple sclerosis:1.39), (FLUOXETINE HYDROCHLORIDEOLANZAPINE:Multiple sclerosis:1.63), (FLUPENTIXOL:Multiple sclerosis:0.3), (FLUPENTIXOL DECANOATE:Multiple sclerosis:0.17), (FLUPENTIXOL DIHYDROCHLORIDE:Multiple sclerosis:0.09), (FLUPHENAZINE:Multiple sclerosis:0.93), (FLUTICASONE PROPIONATEFORMOTEROL FUMARATE:Multiple sclerosis:1.11), (FOLIC ACIDIRON:Multiple sclerosis:0.18), (FOLIC ACIDIRONMINERALSVITAMINS:Multiple sclerosis:0.49), (FORMALDEHYDE:Multiple sclerosis:0.03), (FORMOTEROL FUMARATEGLYCOPYRROLATE:Multiple sclerosis:0.02), (FORMOTEROL FUMARATE\MOMETASONE FUROATE:Multiple sclerosis:1.54), (FUSIDATE SODIUM:Multiple sclerosis:0.07), (GADOVERSETAMIDE:Multiple sclerosis:0.65), (GIMERACILOTERACILTEGAFUR:Multiple sclerosis:0.09), (GINSENG EXTRACT:Multiple sclerosis:0.11), (GLIMEPIRIDEMETFORMIN:Multiple sclerosis:0.29), (GLYCOPYRROLATEINDACATEROL MALEATE:Multiple sclerosis:1.34), (GLYCOPYRRONIUM:Multiple sclerosis:1.16), (GLYCOPYRRONIUMINDACATEROL:Multiple sclerosis:2.47), (GOLD:Multiple sclerosis:0.49), (GOLD SODIUM THIOMALATE:Multiple sclerosis:0.07), (GOLDENSEAL:Multiple sclerosis:0.16), (GOTU KOLA:Multiple sclerosis:0.19), (GRAPEFRUIT SEED EXTRACT:Multiple sclerosis:0.13), (GUAIFENESINPHENYLEPHRINE HYDROCHLORIDE:Multiple sclerosis:0.75), (HARPAGOPHYTUM PROCUMBENS ROOT:Multiple sclerosis:0.15), (HEMP:Multiple sclerosis:0.41), (HEPARIN CALCIUM:Multiple sclerosis:0.85), (HERBALSHOMEOPATHICSGOLDENSEAL:Multiple sclerosis:0.49), (HERBALSKARAYA GUM:Multiple sclerosis:0.24), (HERBSUNSPECIFIED INGREDIENT:Multiple sclerosis:0), (HUMAN COLOSTRUM:Multiple sclerosis:0.33), (HYDROCHLOROTHIAZIDEPROPRANOLOL HYDROCHLORIDE:Multiple sclerosis:0.24), (IBRITUMOMAB TIUXETAN:Multiple sclerosis:0.16), (IMIGLUCERASE:Multiple sclerosis:1.43), (IMIPENEM:Multiple sclerosis:0.24), (INDACATEROL MALEATE:Multiple sclerosis:0.58), (INFLIXIMAB-DYYB:Multiple sclerosis:1.7), (INOTUZUMAB OZOGAMICIN:Multiple sclerosis:0.05), (INTERFERON GAMMA-1B:Multiple sclerosis:0.99), (IRINOTECAN HYDROCHLORIDE:Multiple sclerosis:0.84), (ISOFLAVONES SOY:Multiple sclerosis:0.4), (ISONIAZIDPYRAZINAMIDERIFAMPIN:Multiple sclerosis:0.22), (ISOPROPYL ALCOHOLLEUPROLIDE ACETATENORETHINDRONE ACETATE:Multiple sclerosis:0.81), (ITRACONAZOLE\SODIUM CHLORIDE:Multiple sclerosis:0.64), (IVACAFTOR:Multiple sclerosis:0.29), (IXAZOMIB:Multiple sclerosis:0.79), (JWH-018:Multiple sclerosis:0.08), (KERATIN:Multiple sclerosis:0.19), (LANOLINMINERAL OILPETROLATUM:Multiple sclerosis:0.08), (LANREOTIDE:Multiple sclerosis:1.04), (LANREOTIDE ACETATE:Multiple sclerosis:1.05), (LAPINE T-LYMPHOCYTE IMMUNE GLOBULIN:Multiple sclerosis:0.34), (LENVATINIB:Multiple sclerosis:0.26), (LETROZOLERIBOCICLIB:Multiple sclerosis:0.05), (LEVAMLODIPINE:Multiple sclerosis:0.05), (LEVOMEFOLATE CALCIUM\METHYLCOBALAMINPYRIDOXAL 5-PHOSPHATE:Multiple sclerosis:0.17), (LEVOMEFOLIC ACIDMETHYLCOBALAMINTURMERIC:Multiple sclerosis:0.54), (LEVORPHANOL:Multiple sclerosis:0.09), (LITHIUM OROTATE:Multiple sclerosis:0.09), (LOFEPRAMINE:Multiple sclerosis:0.09), (LOMITAPIDE MESYLATE:Multiple sclerosis:2.05), (LURASIDONE:Multiple sclerosis:0.44), (LUTETIUM LU-177:Multiple sclerosis:0.97), (MAGNESIUM GLUCEPTATE:Multiple sclerosis:0.12), (MANIDIPINE HYDROCHLORIDE:Multiple sclerosis:0.15), (RECUTITA:Multiple sclerosis:0.61), (MECLOFENAMATE SODIUM:Multiple sclerosis:0.09), (MEPOLIZUMAB:Multiple sclerosis:1.08), (METFORMIN HYDROCHLORIDESAXAGLIPTIN:Multiple sclerosis:0.87), (METFORMIN PAMOATE:Multiple sclerosis:0.35), (METHYLENE BLUE:Multiple sclerosis:0.11), (METHYLERGONOVINE MALEATE:Multiple sclerosis:0.33), (METHYLPREDNISOLONE OR METHYLPREDNISOLONE ACETATE:Multiple sclerosis:0.16), (METRELEPTIN:Multiple sclerosis:1.2), (MICAFUNGIN:Multiple sclerosis:0.74), (MIGLUSTAT:Multiple sclerosis:0.54), (MIZORIBINE:Multiple sclerosis:0.17), (MORPHINE\MORPHINE HYDROCHLORIDE\MORPHINE SULFATE\MORPHINE TARTRATE:Multiple sclerosis:0.24), (NADH:Multiple sclerosis:0.38), (NALOXONE HYDROCHLORIDEPENTAZOCINE HYDROCHLORIDE:Multiple sclerosis:0.35), (NATTOKINASE:Multiple sclerosis:0.2), (NEOMYCIN:Multiple sclerosis:1.26), (NEOSTIGMINE BROMIDE:Multiple sclerosis:0.09), (NICORANDIL:Multiple sclerosis:0.84), (NIRAPARIB:Multiple sclerosis:1.03), (NUSINERSEN:Multiple sclerosis:0.39), (OLANZAPINE PAMOATE:Multiple sclerosis:0.71), (OLIVE OIL:Multiple sclerosis:0.07), (OLODATEROL HYDROCHLORIDE:Multiple sclerosis:0.67), (OTILONIUM BROMIDE:Multiple sclerosis:0.25), (OXANDROLONE:Multiple sclerosis:0.04), (OXYQUINOLINE SULFATE\SODIUM LAURYL SULFATE:Multiple sclerosis:0.11), (PAMIDRONATE DISODIUM:Multiple sclerosis:1.7), (PANITUMUMAB:Multiple sclerosis:0.45), (PAPAYA:Multiple sclerosis:0.77), (PARATHYROID HORMONE:Multiple sclerosis:0.69), (PASIREOTIDE:Multiple sclerosis:1.2), (PASSIFLORA INCARNATA FLOWER:Multiple sclerosis:0.32), (PEMETREXED:Multiple sclerosis:0.18), (PENBUTOLOL SULFATE:Multiple sclerosis:0.19), (PENTAMIDINE:Multiple sclerosis:0.1), (PENTAZOCINE LACTATE:Multiple sclerosis:1.28), (PEPSIN:Multiple sclerosis:0.86), (PERTUZUMAB:Multiple sclerosis:0.01), (PHENELZINE:Multiple sclerosis:0.77), (PHENYLEPHRINE HYDROCHLORIDEPROMETHAZINE HYDROCHLORIDE:Multiple sclerosis:0.71), (PHENYLEPHRINE HYDROCHLORIDEWITCH HAZEL:Multiple sclerosis:0.68), (PHLOROGLUCINOL1,3,5-TRIMETHOXYBENZENE:Multiple sclerosis:0.13), (PHOSPHATIDYL SERINE:Multiple sclerosis:0.1), (PIPERACILLIN:Multiple sclerosis:0.04), (PITOFENONE:Multiple sclerosis:0.8), (PODOFILOX:Multiple sclerosis:0.57), (POLYVINYL ALCOHOLPOVIDONE:Multiple sclerosis:0.23), (POMALIDOMIDE:Multiple sclerosis:0.43), (PONATINIB:Multiple sclerosis:1.52), (POTASSIUM PHOSPHATE, MONOBASIC:Multiple sclerosis:0.13), (PREDNISOLONE ACETATESULFACETAMIDE SODIUM:Multiple sclerosis:0.6), (PROBUCOL:Multiple sclerosis:0.01), (PROCATEROL HYDROCHLORIDE:Multiple sclerosis:0.33), (PROCYCLIDINE:Multiple sclerosis:0.58), (PROMAZINE:Multiple sclerosis:0.01), (PROPOXYPHENE:Multiple sclerosis:0.48), (PROPOXYPHENE NAPSYLATE:Multiple sclerosis:0.09), (PRUCALOPRIDE:Multiple sclerosis:0.11), (PSORALEN:Multiple sclerosis:0.35), (PYRIDOXAL PHOSPHATE:Multiple sclerosis:0.24), (PYRIMETHAMINE:Multiple sclerosis:0.32), (QUINACRINE:Multiple sclerosis:1.55), (QUINAGOLIDE HYDROCHLORIDE:Multiple sclerosis:0.75), (QUINIDINE GLUCONATE:Multiple sclerosis:0.79), (RADIATION THERAPY:Multiple sclerosis:0.84), (RED CLOVER:Multiple sclerosis:0.74), (RED YEAST:Multiple sclerosis:0.21), (RHUBARB:Multiple sclerosis:0.15), (RILMENIDINE PHOSPHATE:Multiple sclerosis:0.64), (RILPIVIRINE HYDROCHLORIDE:Multiple sclerosis:0.7), (RIOCIGUAT:Multiple sclerosis:0.62), (RUCAPARIB CAMSYLATE:Multiple sclerosis:0.03), (RUFINAMIDE:Multiple sclerosis:0.31), (RUPATADINE FUMARATE:Multiple sclerosis:0.42), (SAFINAMIDE MESYLATE:Multiple sclerosis:0.28), (SAPROPTERIN DIHYDROCHLORIDE:Multiple sclerosis:0.42), (SERRAPEPTASE:Multiple sclerosis:0.53), (SERTINDOLE:Multiple sclerosis:0.33), (SEVELAMER:Multiple sclerosis:0.08), (SHARK CARTILAGESHARK, UNSPECIFIED:Multiple sclerosis:0.06), (SHARK LIVER OIL:Multiple sclerosis:0.02), (SIBUTRAMINE HYDROCHLORIDE:Multiple sclerosis:2.19), (SILICON DIOXIDE:Multiple sclerosis:1.28), (SIPULEUCEL-T:Multiple sclerosis:0.3), (SODIUM HYPOCHLORITE:Multiple sclerosis:0.21), (SODIUM PHOSPHATE, MONOBASIC, ANHYDROUS:Multiple sclerosis:0.12), (SODIUM POLYSTYRENE SULFONATE:Multiple sclerosis:0.49), (SULFANILAMIDE:Multiple sclerosis:0.01), (SUPLATAST TOSILATE:Multiple sclerosis:0.32), (TALIGLUCERASE ALFA:Multiple sclerosis:0.35), (TASIMELTEON:Multiple sclerosis:1.35), (TEDUGLUTIDE:Multiple sclerosis:1.45), (TEDUGLUTIDE\WATER:Multiple sclerosis:1.66), (TELOTRISTAT ETHYL:Multiple sclerosis:0.57), (TENOXICAM:Multiple sclerosis:0.67), (TESAMORELIN:Multiple sclerosis:0.83), (TETRACAINE:Multiple sclerosis:0), (TETRACAINE HYDROCHLORIDE:Multiple sclerosis:0.45), (THEANINE:Multiple sclerosis:0.59), (THIORIDAZINE:Multiple sclerosis:0.21), (THYMOL:Multiple sclerosis:0.07), (TIMOLOL MALEATETRAVOPROST:Multiple sclerosis:1.95), (TIMOLOLTRAVOPROST:Multiple sclerosis:0.97), (TINZAPARIN SODIUM:Multiple sclerosis:0.73), (TIOPRONIN:Multiple sclerosis:0.33), (TIPIRACIL HYDROCHLORIDETRIFLURIDINE:Multiple sclerosis:0.24), (TOFACITINIB:Multiple sclerosis:0.63), (TOLMETIN:Multiple sclerosis:0.49), (TOLMETIN SODIUM:Multiple sclerosis:0.45), (TOLVAPTAN:Multiple sclerosis:0.04), (TREPROSTINIL DIOLAMINE:Multiple sclerosis:0.66), (TRIAMCINOLONE DIACETATE:Multiple sclerosis:0.09), (TRIFLUOPERAZINE HYDROCHLORIDE:Multiple sclerosis:0.7), (TRIPTORELIN PAMOATE:Multiple sclerosis:1.29), (VASOPRESSIN:Multiple sclerosis:0.59), (VELAGLUCERASE ALFA:Multiple sclerosis:0.11), (VEMURAFENIB:Multiple sclerosis:0.86), (VINCRISTINE:Multiple sclerosis:0.2), (VINCRISTINE SULFATE:Multiple sclerosis:0.48), (VINORELBINE TARTRATE:Multiple sclerosis:0.44), (VORAPAXAR SULFATE:Multiple sclerosis:0.14), (ZINC CITRATE:Multiple sclerosis:0.22), (ZINC PICOLINATE:Multiple sclerosis:0.12), (ZIV-AFLIBERCEPT:Multiple sclerosis:0), (ZOTEPINE:Multiple sclerosis:0.15), (ACETAMINOPHENPROPOXYPHENE HYDROCHLORIDE:Multiple sclerosis relapse:0.12), (AMIODARONE:Multiple sclerosis relapse:0.39), (AMIODARONE HYDROCHLORIDE:Multiple sclerosis relapse:0.09), (ANASTROZOLE:Multiple sclerosis relapse:0.11), (BUDESONIDEFORMOTEROL FUMARATE:Multiple sclerosis relapse:0.04), (BUDESONIDEFORMOTEROL FUMARATE DIHYDRATE:Multiple sclerosis relapse:0.17), (CARVEDILOL PHOSPHATE:Multiple sclerosis relapse:0.11), (CERTOLIZUMAB PEGOL:Multiple sclerosis relapse:1.09), (COLCHICINE:Multiple sclerosis relapse:0.45), (DOXAZOSINDOXAZOSIN MESYLATE:Multiple sclerosis relapse:0.06), (ETORICOXIB:Multiple sclerosis relapse:0.24), (EVOLOCUMAB:Multiple sclerosis relapse:0.72), (FEBUXOSTAT:Multiple sclerosis relapse:0.35), (HYDROCHLOROTHIAZIDEIRBESARTAN:Multiple sclerosis relapse:0.11), (HYOSCYAMINE:Multiple sclerosis relapse:0.26), (IBRUTINIB:Multiple sclerosis relapse:0.68), (INDAPAMIDE:Multiple sclerosis relapse:0.29), (ISOSORBIDE MONONITRATE:Multiple sclerosis relapse:0.59), (LABETALOLLABETALOL HYDROCHLORIDE:Multiple sclerosis relapse:0.36), (LERCANIDIPINE:Multiple sclerosis relapse:0.14), (METAXALONE:Multiple sclerosis relapse:0.82), (METHADONE HYDROCHLORIDE:Multiple sclerosis relapse:0.2), (NALOXONE HYDROCHLORIDEOXYCODONE HYDROCHLORIDE:Multiple sclerosis relapse:0.17), (OLANZAPINE:Multiple sclerosis relapse:0.68), (PANTOPRAZOLE MAGNESIUM:Multiple sclerosis relapse:0.15), (PENICILLIN:Multiple sclerosis relapse:0.31), (PHENOBARBITAL:Multiple sclerosis relapse:0.33), (PRAMIPEXOLE:Multiple sclerosis relapse:0.69), (RANOLAZINE:Multiple sclerosis relapse:0.01), (RISEDRONATE SODIUM:Multiple sclerosis relapse:0.13), (SACUBITRILVALSARTAN:Multiple sclerosis relapse:0.47), (SODIUM OXYBATE:Multiple sclerosis relapse:1.12), (ZINC:Multiple sclerosis relapse:0.17), (ALBIGLUTIDE:Muscle disorder:0.06), (BISACODYL OR DOCUSATE SODIUM:Muscle disorder:0.47), (BUPRENORPHINE HYDROCHLORIDENALOXONE HYDROCHLORIDE:Muscle disorder:0.07), (CALCIUM CARBONATECHOLECALCIFEROL:Muscle disorder:0.58), (CANDESARTAN:Muscle disorder:0.08), (CANDESARTAN CILEXETIL:Muscle disorder:0.18), (CARVEDILOL PHOSPHATE:Muscle disorder:0.35), (CEPHALEXIN:Muscle disorder:0.37), (CERTOLIZUMAB PEGOL:Muscle disorder:0.43), (CLINDAMYCINCLINDAMYCIN PHOSPHATE:Muscle disorder:0.43), (CLOTRIMAZOLE:Muscle disorder:0.06), (DACLIZUMAB:Muscle disorder:0.27), (DAPAGLIFLOZIN PROPANEDIOL:Muscle disorder:0.08), (DASATINIB:Muscle disorder:0.02), (DEVICE:Muscle disorder:0.34), (DEXAMETHASONE:Muscle disorder:0.12), (DUTASTERIDE:Muscle disorder:0.23), (ESZOPICLONE:Muscle disorder:0.69), (EVEROLIMUS:Muscle disorder:0.23), (FERROUS SULFATEFERROUS SULFATE, DRIED:Muscle disorder:0.53), (HEPARIN SODIUM:Muscle disorder:0.16), (HERBALSTURMERIC:Muscle disorder:0.27), (HYDRALAZINE HYDROCHLORIDE:Muscle disorder:0.22), (HYDROCHLOROTHIAZIDEVALSARTAN:Muscle disorder:0.16), (HYDROXYCHLOROQUINE DIPHOSPHATE:Muscle disorder:0.02), (HYDROXYZINE PAMOATE:Muscle disorder:0.84), (HYOSCYAMINE:Muscle disorder:0.51), (LABETALOLLABETALOL HYDROCHLORIDE:Muscle disorder:0.09), (METHENAMINE HIPPURATE:Muscle disorder:0.14), (METHYLPHENIDATE HYDROCHLORIDE:Muscle disorder:0.49), (METHYLPREDNISOLONE ACETATE:Muscle disorder:0.28), (METHYLPREDNISOLONE SODIUM SUCCINATE:Muscle disorder:0.9), (NITROGLYCERIN:Muscle disorder:0.41), (NORTRIPTYLINE:Muscle disorder:0.58), (OLANZAPINE:Muscle disorder:0.16), (OMEGA-3-ACID ETHYL ESTERS:Muscle disorder:0.31), (PAROXETINEPAROXETINE HYDROCHLORIDE:Muscle disorder:0.36), (PEGINTERFERON BETA-1A:Muscle disorder:0.39), (PENICILLIN:Muscle disorder:0.09), (PRAMIPEXOLE DIHYDROCHLORIDE:Muscle disorder:0.83), (PRIMIDONE:Muscle disorder:0.73), (PROGESTERONE:Muscle disorder:0), (QUETIAPINE:Muscle disorder:0.09), (RABEPRAZOLE SODIUM:Muscle disorder:0.02), (RALOXIFENE HYDROCHLORIDE:Muscle disorder:0.23), (RISEDRONATE SODIUM:Muscle disorder:0.37), (SACUBITRILVALSARTAN:Muscle disorder:0.57), (SOLIFENACIN SUCCINATE:Muscle disorder:0), (SOTALOL:Muscle disorder:0.04), (SUMATRIPTANSUMATRIPTAN SUCCINATE:Muscle disorder:0.15), (TOLTERODINE TARTRATE:Muscle disorder:0.32), (UMECLIDINIUM BROMIDEVILANTEROL TRIFENATATE:Muscle disorder:0.28), (VILAZODONE HYDROCHLORIDE:Muscle disorder:0.18), (ZONISAMIDE:Muscle disorder:0.04), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Muscle relaxant therapy:0.46), (.ALPHA.1-PROTEINASE INHIBITOR HUMAN:Muscle relaxant therapy:0.39), (ACENOCOUMAROL:Muscle relaxant therapy:0.27), (ACETYLCYSTEINE:Muscle relaxant therapy:0.12), (ADO-TRASTUZUMAB EMTANSINE:Muscle relaxant therapy:0.15), (AGALSIDASE BETA:Muscle relaxant therapy:0.67), (ALBIGLUTIDE:Muscle relaxant therapy:0.52), (ALCOHOL:Muscle relaxant therapy:1.34), (ALENDRONIC ACID:Muscle relaxant therapy:0.85), (ALFACALCIDOL:Muscle relaxant therapy:0.32), (ALISKIREN HEMIFUMARATE:Muscle relaxant therapy:0.86), (AMINO ACIDSDEXTROSEELECTROLYTES NOS:Muscle relaxant therapy:0.61), (AMISULPRIDE:Muscle relaxant therapy:0.76), (AMLODIPINE BESYLATEHYDROCHLOROTHIAZIDEVALSARTAN:Muscle relaxant therapy:0.25), (AMMONIUM CHLORIDEDEXTROMETHORPHAN\SODIUM CITRATE:Muscle relaxant therapy:0.34), (AMMONIUM LACTATE:Muscle relaxant therapy:0.83), (AMPICILLIN SODIUMSULBACTAM SODIUM:Muscle relaxant therapy:0.24), (ANTIHISTAMINES NOS:Muscle relaxant therapy:0.43), (ARIPIPRAZOLE LAUROXIL:Muscle relaxant therapy:0.89), (BECLOMETHASONE:Muscle relaxant therapy:0.08), (BETAMETHASONECALCIPOTRIENE:Muscle relaxant therapy:0.21), (BETHANECHOL CHLORIDE:Muscle relaxant therapy:0.12), (BROTIZOLAM:Muscle relaxant therapy:0.05), (CALCIPOTRIENE:Muscle relaxant therapy:0.27), (CALCIUM\MAGNESIUM:Muscle relaxant therapy:0.66), (CARBIDOPA:Muscle relaxant therapy:0.27), (CARVEDILOL PHOSPHATE:Muscle relaxant therapy:1.97), (CEFOTAXIME SODIUM:Muscle relaxant therapy:0.39), (CEFUROXIME AXETIL:Muscle relaxant therapy:0.33), (CILASTATIN SODIUMIMIPENEM:Muscle relaxant therapy:0.31), (CLOMIPRAMINE HYDROCHLORIDE:Muscle relaxant therapy:0.73), (DABIGATRAN:Muscle relaxant therapy:0.74), (DACLIZUMAB:Muscle relaxant therapy:1.49), (DALTEPARIN:Muscle relaxant therapy:0.69), (DANAZOL:Muscle relaxant therapy:0.57), (DAPAGLIFLOZIN PROPANEDIOLMETFORMIN HYDROCHLORIDE:Muscle relaxant therapy:0.05), (DESMOPRESSIN:Muscle relaxant therapy:0.51), (DESOGESTRELETHINYL ESTRADIOL:Muscle relaxant therapy:0.04), (DESVENLAFAXINE:Muscle relaxant therapy:0.2), (DEXMETHYLPHENIDATE HYDROCHLORIDE:Muscle relaxant therapy:0.2), (DIPYRIDAMOLE:Muscle relaxant therapy:0.17), (DIPYRONEPROMETHAZINE HYDROCHLORIDE:Muscle relaxant therapy:0.14), (DOBUTAMINE HYDROCHLORIDE:Muscle relaxant therapy:0.3), (DORZOLAMIDE:Muscle relaxant therapy:0.01), (DORZOLAMIDE HYDROCHLORIDE:Muscle relaxant therapy:0.04), (DROXIDOPA:Muscle relaxant therapy:0.06), (DUTASTERIDETAMSULOSIN HYDROCHLORIDE:Muscle relaxant therapy:0.4), (ECONAZOLE:Muscle relaxant therapy:0.12), (EDOXABAN TOSYLATE:Muscle relaxant therapy:0.87), (EMPAGLIFLOZINLINAGLIPTIN:Muscle relaxant therapy:1.15), (EPINEPHRINELIDOCAINE:Muscle relaxant therapy:0.21), (FENOTEROL HYDROBROMIDE:Muscle relaxant therapy:0.04), (FLUNITRAZEPAM:Muscle relaxant therapy:0.04), (FLUTICASONE FUROATESALMETEROL XINAFOATE:Muscle relaxant therapy:1.03), (FLUVOXAMINE MALEATE:Muscle relaxant therapy:0.84), (FONDAPARINUX SODIUM:Muscle relaxant therapy:0.05), (FORMOTEROL:Muscle relaxant therapy:0.35), (FORMOTEROL FUMARATE:Muscle relaxant therapy:0.93), (FORMOTEROL FUMARATE\MOMETASONE FUROATE:Muscle relaxant therapy:0.53), (FUSIDIC ACID:Muscle relaxant therapy:0.81), (GINGER:Muscle relaxant therapy:0.15), (GLIPIZIDEMETFORMIN HYDROCHLORIDE:Muscle relaxant therapy:0.02), (GLYCOPYRROLATEINDACATEROL MALEATE:Muscle relaxant therapy:0.21), (GLYCOPYRRONIUMINDACATEROL:Muscle relaxant therapy:0.91), (GOSERELIN ACETATE:Muscle relaxant therapy:0.01), (GUANFACINE HYDROCHLORIDE:Muscle relaxant therapy:1.08), (HYDROXYCHLOROQUINE DIPHOSPHATE:Muscle relaxant therapy:0.56), (HYDROXYUREA:Muscle relaxant therapy:0.25), (ILOPROST:Muscle relaxant therapy:0.44), (IMATINIB MESYLATE:Muscle relaxant therapy:0.58), (IMIPRAMINE HYDROCHLORIDE:Muscle relaxant therapy:1.64), (INDACATEROL:Muscle relaxant therapy:0.26), (INDAPAMIDEPERINDOPRIL:Muscle relaxant therapy:0.68), (INFLIXIMAB-DYYB:Muscle relaxant therapy:0.02), (INSULIN BEEF:Muscle relaxant therapy:0.43), (INTERFERON:Muscle relaxant therapy:0.2), (IODINE:Muscle relaxant therapy:0.23), (IRONVITAMINS:Muscle relaxant therapy:0.03), (RHAMNOSUS:Muscle relaxant therapy:0.76), (LOMITAPIDE MESYLATE:Muscle relaxant therapy:0.88), (MEPOLIZUMAB:Muscle relaxant therapy:0.29), (METFORMIN HYDROCHLORIDEVILDAGLIPTIN:Muscle relaxant therapy:0.18), (METHENAMINE HIPPURATE:Muscle relaxant therapy:1.81), (METHYLPREDNISOLONE ACETATE:Muscle relaxant therapy:1.89), (MIDODRINE HYDROCHLORIDE:Muscle relaxant therapy:0.68), (MOEXIPRIL HYDROCHLORIDE:Muscle relaxant therapy:0.27), (OCTREOTIDE ACETATE:Muscle relaxant therapy:0.61), (OMEPRAZOLE\SODIUM BICARBONATE:Muscle relaxant therapy:0.02), (OSELTAMIVIR:Muscle relaxant therapy:0.27), (OSPEMIFENE:Muscle relaxant therapy:0.94), (OXYBUTYNINOXYBUTYNIN CHLORIDE:Muscle relaxant therapy:0.28), (PAMIDRONATE DISODIUM:Muscle relaxant therapy:1.01), (PANCRELIPASE:Muscle relaxant therapy:0.84), (PANTOPRAZOLE MAGNESIUM:Muscle relaxant therapy:1.6), (PARATHYROID HORMONE:Muscle relaxant therapy:0.14), (PARICALCITOL:Muscle relaxant therapy:0.01), (PASIREOTIDE:Muscle relaxant therapy:0.24), (PATIROMER:Muscle relaxant therapy:0.24), (PAZOPANIB HYDROCHLORIDE:Muscle relaxant therapy:0.77), (PEGFILGRASTIM:Muscle relaxant therapy:0.36), (PENICILLIN:Muscle relaxant therapy:2.04), (PENICILLIN G:Muscle relaxant therapy:0.02), (PENICILLIN V:Muscle relaxant therapy:0.14), (PENTOBARBITAL:Muscle relaxant therapy:0.19), (PIMECROLIMUS:Muscle relaxant therapy:0.3), (PONATINIB:Muscle relaxant therapy:1.05), (PREDNISOLONE ACETATE:Muscle relaxant therapy:0.57), (PREDNISOLONE SODIUM PHOSPHATE:Muscle relaxant therapy:0.53), (PREDNISOLONEPREDNISOLONE ACETATEPREDNISOLONE SODIUM SUCCINATE:Muscle relaxant therapy:0.21), (PROTRIPTYLINE HYDROCHLORIDE:Muscle relaxant therapy:0.92), (PYRIDOSTIGMINE BROMIDE:Muscle relaxant therapy:0.65), (QUINAPRIL:Muscle relaxant therapy:0.56), (RIFAMPIN:Muscle relaxant therapy:0.79), (RIVASTIGMINE:Muscle relaxant therapy:0.79), (RIVASTIGMINE TARTRATE:Muscle relaxant therapy:1.13), (SELEGILINE:Muscle relaxant therapy:0.4), (SODIUM CITRATE:Muscle relaxant therapy:0.14), (SODIUM PHOSPHATE, DIBASIC\SODIUM PHOSPHATE, MONOBASIC:Muscle relaxant therapy:0.74), (SUFENTANIL:Muscle relaxant therapy:1.6), (SUFENTANIL CITRATE:Muscle relaxant therapy:0.83), (SULFAMETHOXAZOLE:Muscle relaxant therapy:0.9), (TAMOXIFEN CITRATE:Muscle relaxant therapy:0.08), (TESTOSTERONE UNDECANOATE:Muscle relaxant therapy:0.18), (TETRABENAZINE:Muscle relaxant therapy:0.17), (TETRACYCLINE:Muscle relaxant therapy:1.86), (THIAMYLAL SODIUM:Muscle relaxant therapy:0.39), (TIMOLOL MALEATE:Muscle relaxant therapy:0.61), (TRANDOLAPRIL:Muscle relaxant therapy:0.32), (TRIMIPRAMINE MALEATE:Muscle relaxant therapy:0.24), (UMECLIDINIUM BROMIDEVILANTEROL TRIFENATATE:Muscle relaxant therapy:1.47), (VARDENAFIL HYDROCHLORIDE TRIHYDRATE:Muscle relaxant therapy:0.2), (VASOPRESSIN:Muscle relaxant therapy:0.34), (.ALPHA.-TOCOPHEROLVITAMINS:Muscle spasms:0.35), (ABACAVIR SULFATEDOLUTEGRAVIR SODIUMLAMIVUDINE:Muscle spasms:0.34), (ACECLOFENAC:Muscle spasms:0.32), (ACETAMINOPHEN\CAFFEINEOPIUM:Muscle spasms:0.41), (AJMALINE:Muscle spasms:0.24), (ALBUTEROLIPRATROPIUM BROMIDE:Muscle spasms:0.17), (ALDESLEUKIN:Muscle spasms:0.06), (ALISKIREN HEMIFUMARATEHYDROCHLOROTHIAZIDE:Muscle spasms:0.06), (AMINO ACIDSDEXTROSEELECTROLYTES NOSSOYBEAN OIL:Muscle spasms:0.54), (AMINOPHYLLINE:Muscle spasms:0.27), (AMISULPRIDE:Muscle spasms:1.58), (AMLODIPINEPERINDOPRIL:Muscle spasms:0.51), (AMLODIPINEVALSARTAN:Muscle spasms:1.58), (AMOXICILLINCLARITHROMYCINESOMEPRAZOLE:Muscle spasms:0.92), (AMPHETAMINE SULFATE:Muscle spasms:0.02), (ANAGRELIDE HYDROCHLORIDE:Muscle spasms:0.24), (ANHYDROUS CITRIC ACIDASPIRIN\SODIUM BICARBONATE:Muscle spasms:0.8), (ASCORBIC ACID/CHROMIUM/SELENIUM/VANADYL SULFATE:Muscle spasms:0.66), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Muscle spasms:0.42), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE MONONITRATE:Muscle spasms:0.24), (ASCORBIC ACIDBIOTINCYANOCOBALAMINFOLIC ACIDNIACINPANTOTHENIC ACIDPYRIDOXINE\RIBOFLAVINTHIAMINE:Muscle spasms:0.34), (ASPIRIN\CAFFEINESALICYLAMIDE:Muscle spasms:0.43), (ASPIRINCLOPIDOGREL:Muscle spasms:0.11), (ASPIRINDIHYDROXYALUMINUM AMINOACETATE ANHYDROUSMAGNESIUM CARBONATE:Muscle spasms:0.66), (ASUNAPREVIR:Muscle spasms:0.01), (ATOVAQUONE:Muscle spasms:1.14), (AVANAFIL:Muscle spasms:0.43), (AZTREONAM:Muscle spasms:0.22), (BACITRACIN ZINC:Muscle spasms:0.15), (BAMIFYLLINE HYDROCHLORIDE:Muscle spasms:0.58), (BECLOMETHASONE:Muscle spasms:1.62), (BECLOMETHASONE DIPROPIONATEFORMOTEROL:Muscle spasms:0.02), (BELATACEPT:Muscle spasms:0.38), (BENZOCAINE:Muscle spasms:0.58), (BEPRIDIL:Muscle spasms:0.09), (BERAPROST:Muscle spasms:0.2), (BERAPROST SODIUM:Muscle spasms:1.09), (BETAMETHASONE DIPROPIONATECALCIPOTRIENE HYDRATE:Muscle spasms:0.05), (BIFIDOBACTERIUM SPP.:Muscle spasms:0.86), (CAFFEINEDIPYRONEORPHENADRINE:Muscle spasms:0.17), (CAJUPUT OILEUCALYPTUS OILPEPPERMINT OIL:Muscle spasms:0.11), (CALCIUM CARBONATECHOLECALCIFEROLMAGNESIUM CARBONATE:Muscle spasms:0.13), (CALCIUM CARBONATEETIDRONATE DISODIUM:Muscle spasms:0.19), (CALCIUM GLYCEROPHOSPHATE:Muscle spasms:0.03), (CALCIUM PANTOTHENATECYANOCOBALAMINNIACINAMIDEPYRIDOXINE HYDROCHLORIDE\RIBOFLAVINTHIAMINE MONONITRATE:Muscle spasms:0.58), (CARBAZOCHROME SODIUM SULFONATE:Muscle spasms:0.1), (CARBOXYMETHYLCELLULOSE SODIUMGLYCERINPOLYSORBATE 80:Muscle spasms:0.29), (CASPOFUNGIN:Muscle spasms:0.43), (CEFOTAXIMECEFOTAXIME SODIUM:Muscle spasms:0.22), (CEFTAZIDIME SODIUM:Muscle spasms:0.05), (CERIVASTATIN SODIUM:Muscle spasms:0.49), (CERTOPARIN SODIUM:Muscle spasms:0.11), (CHONDROITIN SULFATE (CHICKEN):Muscle spasms:0.04), (CICLOPIROX OLAMINE:Muscle spasms:0.44), (CIPROFIBRATE:Muscle spasms:1.15), (CLEMASTINE FUMARATE:Muscle spasms:0.27), (CLIKSTAR:Muscle spasms:0.53), (COBICISTATELVITEGRAVIREMTRICITABINETENOFOVIR DISOPROXIL FUMARATE:Muscle spasms:0.23), (CODEINE PHOSPHATEPROMETHAZINE:Muscle spasms:0.02), (CODEINE PHOSPHATEPROMETHAZINE HYDROCHLORIDE:Muscle spasms:0.04), (CRANBERRY CONCENTRATE:Muscle spasms:0.08), (CROTALIDAE POLYVALENT IMMUNE FAB (OVINE):Muscle spasms:0.04), (DABRAFENIB:Muscle spasms:1.03), (DESFLURANE:Muscle spasms:0.15), (DEXAMETHASONE ACETATE:Muscle spasms:1.26), (DIHYDROERGOTAMINE MESYLATE:Muscle spasms:0.19), (DIPYRONE:Muscle spasms:1.67), (DIPYRONEPROMETHAZINE HYDROCHLORIDE:Muscle spasms:0.8), (DISOPYRAMIDE PHOSPHATE:Muscle spasms:0.14), (DOXYLAMINE SUCCINATEPYRIDOXINE HYDROCHLORIDE:Muscle spasms:0.57), (DUTASTERIDETAMSULOSIN HYDROCHLORIDE:Muscle spasms:1.32), (EDOXABAN TOSYLATE:Muscle spasms:2.49), (ELBASVIRGRAZOPREVIR:Muscle spasms:0.43), (ELTROMBOPAG OLAMINE:Muscle spasms:1.17), (EPHEDRINE HYDROCHLORIDEGUAIFENESIN:Muscle spasms:0.47), (EPIRUBICIN:Muscle spasms:0.95), (ERIBULIN:Muscle spasms:0.07), (ERYTHROMYCIN LACTOBIONATE:Muscle spasms:0.14), (ESTRADIOL ACETATE:Muscle spasms:0.19), (ESTRADIOL BENZOATE:Muscle spasms:0.71), (ESTRADIOLLEVONORGESTREL:Muscle spasms:0.38), (ETHAMBUTOL HYDROCHLORIDE:Muscle spasms:0.06), (ETHINYL ESTRADIOLETHYNODIOL DIACETATE:Muscle spasms:0.19), (ETHINYL ESTRADIOLGESTODENE:Muscle spasms:0.55), (ETHOSUXIMIDE:Muscle spasms:0.4), (ETRETINATE:Muscle spasms:1.58), (EUCALYPTOLMENTHOLMETHYL SALICYLATETHYMOL:Muscle spasms:0.35), (FELBAMATE:Muscle spasms:1.05), (FENOPROFEN CALCIUM:Muscle spasms:0.25), (FERROUS BISGLYCINATE:Muscle spasms:0.77), (FESOTERODINE:Muscle spasms:0.08), (FILGRASTIM-SNDZ:Muscle spasms:0.15), (FLUNARIZINE HYDROCHLORIDE:Muscle spasms:0.48), (FLURAZEPAM HYDROCHLORIDE:Muscle spasms:0.13), (FLUTAMIDE:Muscle spasms:0.01), (FLUTICASONE PROPIONATEFORMOTEROL FUMARATE:Muscle spasms:0.68), (FOLIC ACIDIRONMINERALSVITAMINS:Muscle spasms:0.04), (FONDAPARINUX:Muscle spasms:0.56), (GADOBUTROL:Muscle spasms:0.13), (GEFITINIB:Muscle spasms:0.01), (GIMERACILOTERACILTEGAFUR:Muscle spasms:0.77), (GOLD:Muscle spasms:0.13), (GOSERELIN ACETATE:Muscle spasms:0.14), (GUAIFENESINPHENYLEPHRINE HYDROCHLORIDE:Muscle spasms:0.28), (HALCINONIDE:Muscle spasms:0.07), (HALOPERIDOL DECANOATE:Muscle spasms:0.4), (HEPARIN CALCIUM:Muscle spasms:0.6), (HERBALSMINERALSVITAMINS:Muscle spasms:0.39), (HIGH DENSITY POLYETHYLENE:Muscle spasms:0.01), (IBRITUMOMAB TIUXETAN:Muscle spasms:0.14), (IMIPENEM:Muscle spasms:0.44), (INDAPAMIDEPERINDOPRIL:Muscle spasms:1.85), (INFLIXIMAB-DYYB:Muscle spasms:0.49), (INOTUZUMAB OZOGAMICIN:Muscle spasms:0.46), (INSULIN PORKINSULIN PURIFIED PORK:Muscle spasms:0.19), (INTERFERON ALFA-2A:Muscle spasms:1.51), (INTERFERON ALFA-2A OR INTERFERON ALFA-2B:Muscle spasms:0.2), (INTERFERON ALFA-2B:Muscle spasms:1.02), (INTERFERON GAMMA-1B:Muscle spasms:1.44), (IODINE\POTASSIUM IODIDE:Muscle spasms:0.1), (IRON POLYMALTOSE:Muscle spasms:0.21), (ISOPROPYL ALCOHOLLEUPROLIDE ACETATENORETHINDRONE ACETATE:Muscle spasms:0.18), (LAFUTIDINE:Muscle spasms:0.49), (LANREOTIDE:Muscle spasms:0.55), (LAPATINIB DITOSYLATE:Muscle spasms:1.14), (LATANOPROSTTIMOLOL:Muscle spasms:0.44), (LENVATINIB:Muscle spasms:0.66), (LEVOLEUCOVORIN CALCIUM:Muscle spasms:0.8), (LUTETIUM LU-177:Muscle spasms:0.43), (MECLOZINE DIHYDROCHLORIDE:Muscle spasms:0.18), (MEMANTINE:Muscle spasms:0.47), (MENAQUINONE 6:Muscle spasms:0.43), (MEPOLIZUMAB:Muscle spasms:1.41), (METFORMIN HYDROCHLORIDESAXAGLIPTIN:Muscle spasms:0.25), (METHENAMINE MANDELATE:Muscle spasms:0.89), (METHENOLONE ENANTHATE:Muscle spasms:0.8), (METILDIGOXIN:Muscle spasms:0.02), (METRELEPTIN:Muscle spasms:0.35), (MEXILETINE HYDROCHLORIDE:Muscle spasms:0.06), (MINERALS:Muscle spasms:0.2), (MOEXIPRIL HYDROCHLORIDE:Muscle spasms:1.03), (NAFTIFINE HYDROCHLORIDE:Muscle spasms:0.09), (NALOXONE HYDROCHLORIDETILIDINE HYDROCHLORIDE:Muscle spasms:1.42), (NEPAFENAC:Muscle spasms:0.38), (NIMESULIDE:Muscle spasms:0.29), (NISOLDIPINE:Muscle spasms:0.29), (NOREPINEPHRINE:Muscle spasms:0.69), (NOREPINEPHRINE BITARTRATE:Muscle spasms:1.06), (OCTREOTIDE:Muscle spasms:0.76), (OLANZAPINE PAMOATE:Muscle spasms:0.01), (OMEGA-6 FATTY ACIDS:Muscle spasms:0.19), (ORLISTAT:Muscle spasms:0.01), (PAPAYA:Muscle spasms:0.47), (PARICALCITOL:Muscle spasms:1.04), (PASIREOTIDE:Muscle spasms:0.25), (PENICILLAMINE:Muscle spasms:0.09), (PENTAMIDINE ISETHIONATE:Muscle spasms:0.55), (PENTOSTATIN:Muscle spasms:0.67), (PEPSIN:Muscle spasms:0), (PERMETHRIN:Muscle spasms:0.26), (PHENOBARBITAL SODIUM:Muscle spasms:0.57), (PIMOBENDAN:Muscle spasms:0.31), (PINDOLOL:Muscle spasms:0.55), (PIPERACILLIN:Muscle spasms:0.11), (PLERIXAFOR:Muscle spasms:0.01), (POLYVINYL ALCOHOLPOVIDONE:Muscle spasms:0.07), (PORFIMER SODIUM:Muscle spasms:0.19), (PRAMLINTIDE ACETATE:Muscle spasms:0.37), (PRAMOXINE HYDROCHLORIDE:Muscle spasms:0.48), (PRANLUKAST:Muscle spasms:0.54), (PREGNENOLONE:Muscle spasms:0.72), (PROPAFENONE:Muscle spasms:0.89), (PROPATYL NITRATE:Muscle spasms:0.05), (PRUCALOPRIDE SUCCINATE:Muscle spasms:0.4), (PYRIDOSTIGMINE:Muscle spasms:0.31), (QUINIDINE GLUCONATE:Muscle spasms:0.1), (RADIATION THERAPY:Muscle spasms:1.55), (RAMUCIRUMAB:Muscle spasms:0.64), (RILMENIDINE PHOSPHATE:Muscle spasms:0.31), (RUFINAMIDE:Muscle spasms:0.61), (SAPROPTERIN DIHYDROCHLORIDE:Muscle spasms:0.34), (SELEXIPAG:Muscle spasms:0.84), (SEVELAMER:Muscle spasms:0.19), (SIBUTRAMINE HYDROCHLORIDE:Muscle spasms:0.96), (SIPULEUCEL-T:Muscle spasms:0.81), (SODIUM CITRATE:Muscle spasms:0.2), (SODIUM FERROUS CITRATE:Muscle spasms:0.63), (SOTALOL HYDROCHLORIDE:Muscle spasms:0.97), (SPIRULINA:Muscle spasms:0.11), (SULFACETAMIDE SODIUMSULFUR:Muscle spasms:0.01), (SULPIRIDE:Muscle spasms:0.27), (SULTOPRIDE HYDROCHLORIDE:Muscle spasms:0.07), (SUPLATAST TOSILATE:Muscle spasms:0.21), (TAFLUPROST:Muscle spasms:0.34), (TAZAROTENE:Muscle spasms:0.23), (TBO-FILGRASTIM:Muscle spasms:0.07), (TEDUGLUTIDE:Muscle spasms:0.75), (TELOTRISTAT ETHYL:Muscle spasms:0.76), (TEMSIROLIMUS:Muscle spasms:0.07), (TESTOSTERONE UNDECANOATE:Muscle spasms:1.04), (THEOPHYLLINE:Muscle spasms:1.07), (THIOTHIXENE:Muscle spasms:0.26), (TIBOLONE:Muscle spasms:0.81), (TIGECYCLINE:Muscle spasms:0.46), (TILIDINE:Muscle spasms:0.08), (TIOPRONIN:Muscle spasms:0.07), (TRICHLORMETHIAZIDE:Muscle spasms:0.64), (TRIMIPRAMINE MALEATE:Muscle spasms:1.19), (TRIPTORELIN PAMOATE:Muscle spasms:0.58), (ZIDOVUDINE:Muscle spasms:0.12), (ZILEUTON:Muscle spasms:1.43), (.ALPHA.1-PROTEINASE INHIBITOR HUMAN:Muscle spasticity:0.2), (ABIRATERONE ACETATE:Muscle spasticity:0.09), (ACETAMINOPHENPROPOXYPHENE HYDROCHLORIDE:Muscle spasticity:0.71), (ACLIDINIUM BROMIDE:Muscle spasticity:0.39), (AGALSIDASE BETA:Muscle spasticity:0.4), (ALCOHOL:Muscle spasticity:0.7), (ALISKIREN HEMIFUMARATE:Muscle spasticity:0.46), (AMISULPRIDE:Muscle spasticity:0.49), (AMLODIPINE BESYLATEHYDROCHLOROTHIAZIDEOLMESARTAN MEDOXOMIL:Muscle spasticity:0.17), (AMLODIPINEVALSARTAN:Muscle spasticity:0.17), (ANTIHISTAMINES NOS:Muscle spasticity:0.53), (BENSERAZIDELEVODOPA:Muscle spasticity:0.05), (BENZTROPINE MESYLATE:Muscle spasticity:0.22), (BRINZOLAMIDE:Muscle spasticity:0.2), (BRIVARACETAM:Muscle spasticity:0.14), (BUDESONIDEFORMOTEROL FUMARATE:Muscle spasticity:0.58), (BUTYLSCOPOLAMINE BROMIDE:Muscle spasticity:0.74), (CALCITONIN:Muscle spasticity:0.57), (CANAGLIFLOZIN:Muscle spasticity:0.77), (CANAGLIFLOZINMETFORMIN HYDROCHLORIDE:Muscle spasticity:0.37), (CANDESARTAN CILEXETILHYDROCHLOROTHIAZIDE:Muscle spasticity:0.88), (CARBIDOPA:Muscle spasticity:0.35), (CARFILZOMIB:Muscle spasticity:0.66), (CARVEDILOL PHOSPHATE:Muscle spasticity:1.27), (CERTOLIZUMAB PEGOL:Muscle spasticity:0.7), (CETIRIZINE HYDROCHLORIDEPSEUDOEPHEDRINE HYDROCHLORIDE:Muscle spasticity:0.66), (CHLORDIAZEPOXIDE HYDROCHLORIDECLIDINIUM BROMIDE:Muscle spasticity:0.11), (CILASTATIN SODIUMIMIPENEM:Muscle spasticity:0.24), (CODEINE PHOSPHATE:Muscle spasticity:0.03), (CODEINE PHOSPHATEGUAIFENESIN:Muscle spasticity:0.56), (COLCHICINE:Muscle spasticity:0.71), (COLESEVELAM HYDROCHLORIDE:Muscle spasticity:0.39), (DABIGATRAN:Muscle spasticity:0.09), (DANAZOL:Muscle spasticity:0.19), (DARBEPOETIN ALFA:Muscle spasticity:0.17), (DASATINIB:Muscle spasticity:0.52), (DEFLAZACORT:Muscle spasticity:0.49), (DOFETILIDE:Muscle spasticity:0.52), (DOLASETRON MESYLATE:Muscle spasticity:0.19), (DRONEDARONE:Muscle spasticity:0.01), (ECONAZOLE:Muscle spasticity:0.24), (EDOXABAN TOSYLATE:Muscle spasticity:0.51), (ERTAPENEM SODIUM:Muscle spasticity:0.01), (ESOMEPRAZOLE MAGNESIUMNAPROXEN:Muscle spasticity:0.23), (ESTROGENS, CONJUGATEDMEDROXYPROGESTERONE ACETATE:Muscle spasticity:1.23), (EVEROLIMUS:Muscle spasticity:0.36), (EVOLOCUMAB:Muscle spasticity:1.06), (FENOFIBRIC ACID:Muscle spasticity:0.42), (FERROUS FUMARATE:Muscle spasticity:0.07), (FERROUS SULFATEGLYCINE:Muscle spasticity:0.01), (FILGRASTIM:Muscle spasticity:0.11), (FLURAZEPAMFLURAZEPAM HYDROCHLORIDE:Muscle spasticity:0.24), (FLUTICASONE FUROATESALMETEROL XINAFOATE:Muscle spasticity:0.51), (FLUTICASONE FUROATEVILANTEROL TRIFENATATE:Muscle spasticity:0.51), (FORMOTEROL:Muscle spasticity:0.19), (FORMOTEROL FUMARATE\MOMETASONE FUROATE:Muscle spasticity:0.2), (FULVESTRANT:Muscle spasticity:0.15), (FUSIDIC ACID:Muscle spasticity:1.06), (GLYCOPYRRONIUMINDACATEROL:Muscle spasticity:0.54), (HYDROCHLOROTHIAZIDEIRBESARTAN:Muscle spasticity:0.81), (HYDROCHLOROTHIAZIDETELMISARTAN:Muscle spasticity:0.62), (HYDROXYCHLOROQUINE DIPHOSPHATE:Muscle spasticity:0.07), (IBRUTINIB:Muscle spasticity:0.35), (INDAPAMIDE:Muscle spasticity:0.75), (INDAPAMIDEPERINDOPRIL:Muscle spasticity:0.61), (INSULIN BEEF:Muscle spasticity:0.32), (LEDIPASVIRSOFOSBUVIR:Muscle spasticity:0.09), (LERCANIDIPINE HYDROCHLORIDE:Muscle spasticity:0.51), (LEVOMILNACIPRAN HYDROCHLORIDE:Muscle spasticity:0.17), (LIOTHYRONINE SODIUM:Muscle spasticity:0.95), (LOMITAPIDE MESYLATE:Muscle spasticity:0.41), (LURASIDONE HYDROCHLORIDE:Muscle spasticity:0.46), (MEGESTROL ACETATE:Muscle spasticity:0), (METHYLPREDNISOLONE ACETATE:Muscle spasticity:1.81), (METOLAZONE:Muscle spasticity:0.13), (MINOCYCLINEMINOCYCLINE HYDROCHLORIDE:Muscle spasticity:1.14), (MOXIFLOXACIN:Muscle spasticity:0.38), (MOXONIDINE:Muscle spasticity:0.09), (NADROPARIN CALCIUM:Muscle spasticity:0.07), (NATEGLINIDE:Muscle spasticity:0.34), (NILOTINIB:Muscle spasticity:0.48), (OXAPROZIN:Muscle spasticity:0.08), (OXYBUTYNINOXYBUTYNIN CHLORIDE:Muscle spasticity:0.12), (OXYMETAZOLINE:Muscle spasticity:0.16), (OXYMORPHONE:Muscle spasticity:0.33), (PALIPERIDONE PALMITATE:Muscle spasticity:0.01), (PATIROMER:Muscle spasticity:0.02), (PEGFILGRASTIM:Muscle spasticity:0.12), (PENICILLIN:Muscle spasticity:1.52), (PERAMPANEL:Muscle spasticity:0.08), (PILOCARPINE:Muscle spasticity:0.23), (PIROXICAM:Muscle spasticity:0.34), (PITAVASTATIN CALCIUM:Muscle spasticity:0.29), (PREDNISOLONE ACETATE:Muscle spasticity:0.01), (PREDNISOLONEPREDNISOLONE ACETATEPREDNISOLONE SODIUM SUCCINATE:Muscle spasticity:0.03), (PROTRIPTYLINE HYDROCHLORIDE:Muscle spasticity:0.14), (QUINAPRIL:Muscle spasticity:0.13), (RABEPRAZOLE:Muscle spasticity:1.02), (RALOXIFENE HYDROCHLORIDE:Muscle spasticity:0.28), (SECUKINUMAB:Muscle spasticity:0.43), (SOTALOL:Muscle spasticity:1.39), (SULINDAC:Muscle spasticity:0.1), (TEDUGLUTIDE\WATER:Muscle spasticity:0.22), (TERBUTALINE SULFATE:Muscle spasticity:0.98), (TIMOLOL MALEATE:Muscle spasticity:0.13), (TIOTROPIUM:Muscle spasticity:0.52), (TRANDOLAPRIL:Muscle spasticity:0.08), (UMECLIDINIUM BROMIDE:Muscle spasticity:0.12), (VALGANCICLOVIR HYDROCHLORIDE:Muscle spasticity:0.33), (VANCOMYCIN HYDROCHLORIDE:Muscle spasticity:0.42), (VORTIOXETINE HYDROBROMIDE:Muscle spasticity:0.34), (ZILEUTON:Muscle spasticity:0.05), (EVOLOCUMAB:Muscle strain:0.1), (BUDESONIDE:Muscle tightness:0.21), (CERTOLIZUMAB PEGOL:Muscle tightness:0.81), (DIVALPROEX SODIUM:Muscle tightness:0.67), (ENALAPRIL:Muscle tightness:0.37), (ENALAPRIL MALEATE:Muscle tightness:0.09), (EPINEPHRINE:Muscle tightness:0.23), (EVOLOCUMAB:Muscle tightness:0.81), (IBRUTINIB:Muscle tightness:0.24), (METHYLPHENIDATE HYDROCHLORIDE:Muscle tightness:0.24), (METRONIDAZOLE:Muscle tightness:0.02), (PANTOPRAZOLE:Muscle tightness:0.27), (PRAMIPEXOLE:Muscle tightness:0.1), (PRAMIPEXOLE DIHYDROCHLORIDE:Muscle tightness:0.22), (PRIMIDONE:Muscle tightness:0.18), (QUETIAPINE FUMARATE:Muscle tightness:0.1), (RABEPRAZOLE SODIUM:Muscle tightness:0.11), (RAMIPRIL:Muscle tightness:0.16), (SPIRONOLACTONE:Muscle tightness:0.26), (SUMATRIPTANSUMATRIPTAN SUCCINATE:Muscle tightness:0.07), (TERIFLUNOMIDE:Muscle tightness:1.11), (WARFARIN:Muscle tightness:0.27), (WARFARIN SODIUM:Muscle tightness:0.07), (METHYLPREDNISOLONE SODIUM SUCCINATE:Muscle twitching:0.08), (NAPROXEN:Muscle twitching:0.23), (VARENICLINE TARTRATE:Muscle twitching:0.26), (ALBUTEROL SULFATEIPRATROPIUM BROMIDE:Muscular weakness:0.03), (AMIODARONE:Muscular weakness:0.38), (AMPHETAMINE ASPARTATEAMPHETAMINE SULFATEDEXTROAMPHETAMINE SACCHARATEDEXTROAMPHETAMINE SULFATE:Muscular weakness:0.16), (ANASTROZOLE:Muscular weakness:0.69), (CALCIUM CARBONATECHOLECALCIFEROL:Muscular weakness:0.33), (CANDESARTAN CILEXETIL:Muscular weakness:0.2), (COLCHICINE:Muscular weakness:0.12), (DACLIZUMAB:Muscular weakness:0.13), (DEVICE:Muscular weakness:0.08), (DIPHENHYDRAMINE:Muscular weakness:0.41), (DUTASTERIDE:Muscular weakness:0.09), (ESZOPICLONE:Muscular weakness:0.33), (EVEROLIMUS:Muscular weakness:0.12), (EVOLOCUMAB:Muscular weakness:0.61), (FENOFIBRATE:Muscular weakness:0.25), (FULVESTRANT:Muscular weakness:0.59), (GADOVERSETAMIDE:Muscular weakness:0.25), (HEPARIN SODIUM:Muscular weakness:0.07), (HYDROXYZINE PAMOATE:Muscular weakness:0), (IBRUTINIB:Muscular weakness:0.46), (IRBESARTAN:Muscular weakness:0.07), (ISOSORBIDE MONONITRATE:Muscular weakness:0.16), (METHYLPREDNISOLONE ACETATE:Muscular weakness:0.19), (NEBIVOLOL:Muscular weakness:0.25), (OXYCODONE:Muscular weakness:0.01), (OXYGEN:Muscular weakness:0.14), (PANTOPRAZOLE:Muscular weakness:0.76), (PANTOPRAZOLE MAGNESIUM:Muscular weakness:0.07), (POTASSIUM:Muscular weakness:0.83), (PRAMIPEXOLE:Muscular weakness:0.31), (PRAVASTATIN:Muscular weakness:0.58), (RANOLAZINE:Muscular weakness:0.26), (SACUBITRILVALSARTAN:Muscular weakness:0.73), (SODIUM OXYBATE:Muscular weakness:0.22), (TELMISARTAN:Muscular weakness:0.31), (TOCILIZUMAB:Muscular weakness:0.06), (VARENICLINE TARTRATE:Muscular weakness:0.14), (VITAMIN B:Muscular weakness:0.24), (ADO-TRASTUZUMAB EMTANSINE:Musculoskeletal chest pain:0.43), (ALCOHOL:Musculoskeletal chest pain:0.16), (AMIODARONE:Musculoskeletal chest pain:0.8), (AMIODARONE HYDROCHLORIDE:Musculoskeletal chest pain:0.24), (AMPHETAMINE ASPARTATEAMPHETAMINE SULFATEDEXTROAMPHETAMINE SACCHARATEDEXTROAMPHETAMINE SULFATE:Musculoskeletal chest pain:0.01), (APREPITANT:Musculoskeletal chest pain:0.24), (BICALUTAMIDE:Musculoskeletal chest pain:0.77), (BISOPROLOL FUMARATE:Musculoskeletal chest pain:0.35), (BUPROPION:Musculoskeletal chest pain:0.17), (BUSPIRONE HYDROCHLORIDE:Musculoskeletal chest pain:0.11), (CEPHALEXIN:Musculoskeletal chest pain:0.9), (CHOLESTYRAMINE:Musculoskeletal chest pain:0.3), (CLOZAPINE:Musculoskeletal chest pain:0.38), (DIPHENHYDRAMINE:Musculoskeletal chest pain:0.9), (DUTASTERIDE:Musculoskeletal chest pain:0.3), (ENALAPRIL:Musculoskeletal chest pain:1.57), (ESZOPICLONE:Musculoskeletal chest pain:0.28), (EVOLOCUMAB:Musculoskeletal chest pain:0.33), (EZETIMIBESIMVASTATIN:Musculoskeletal chest pain:0), (FLUTICASONE FUROATEVILANTEROL TRIFENATATE:Musculoskeletal chest pain:0.01), (HYDROCHLOROTHIAZIDEVALSARTAN:Musculoskeletal chest pain:0.02), (IMATINIB MESYLATE:Musculoskeletal chest pain:0.23), (INDAPAMIDEPERINDOPRIL:Musculoskeletal chest pain:0.06), (INSULIN DETEMIR:Musculoskeletal chest pain:0.16), (IRBESARTAN:Musculoskeletal chest pain:0.38), (ISOSORBIDE MONONITRATE:Musculoskeletal chest pain:0.22), (LINAGLIPTIN:Musculoskeletal chest pain:0.13), (LOVASTATIN:Musculoskeletal chest pain:0.52), (MOXIFLOXACIN HYDROCHLORIDE:Musculoskeletal chest pain:0.41), (NEBIVOLOL:Musculoskeletal chest pain:0.43), (NILOTINIB:Musculoskeletal chest pain:0.89), (OLANZAPINE:Musculoskeletal chest pain:0.49), (PANTOPRAZOLE MAGNESIUM:Musculoskeletal chest pain:0.16), (PAROXETINEPAROXETINE HYDROCHLORIDE:Musculoskeletal chest pain:0.05), (PEMETREXED DISODIUM:Musculoskeletal chest pain:0.23), (PERINDOPRIL:Musculoskeletal chest pain:0.13), (PERTUZUMAB:Musculoskeletal chest pain:0.15), (PRAVASTATIN:Musculoskeletal chest pain:0.57), (PRIMIDONE:Musculoskeletal chest pain:0.01), (RANOLAZINE:Musculoskeletal chest pain:0.29), (RISEDRONATE SODIUM:Musculoskeletal chest pain:0.15), (SACUBITRILVALSARTAN:Musculoskeletal chest pain:0.96), (SECUKINUMAB:Musculoskeletal chest pain:0.25), (SODIUM OXYBATE:Musculoskeletal chest pain:0.28), (TERIFLUNOMIDE:Musculoskeletal chest pain:0.34), (TIMOLOL:Musculoskeletal chest pain:0.01), (TOCILIZUMAB:Musculoskeletal chest pain:0.24), (TOFACITINIB CITRATE:Musculoskeletal chest pain:0.34), (TRASTUZUMAB:Musculoskeletal chest pain:0.37), (TRIAMTERENE:Musculoskeletal chest pain:0.05), (UMECLIDINIUM BROMIDEVILANTEROL TRIFENATATE:Musculoskeletal chest pain:0.22), (VANCOMYCIN HYDROCHLORIDE:Musculoskeletal chest pain:0.03), (VITAMIN B:Musculoskeletal chest pain:0.17), (ACETAMINOPHENBUTALBITALCAFFEINE:Musculoskeletal discomfort:0.04), (AMOXICILLIN:Musculoskeletal discomfort:0.14), (BISACODYL OR DOCUSATE SODIUM:Musculoskeletal discomfort:0.01), (BISOPROLOL FUMARATE:Musculoskeletal discomfort:0.16), (BUPRENORPHINE HYDROCHLORIDENALOXONE HYDROCHLORIDE:Musculoskeletal discomfort:0.01), (CALCIUM CITRATE:Musculoskeletal discomfort:0.32), (CEPHALEXIN:Musculoskeletal discomfort:0.04), (CERTOLIZUMAB PEGOL:Musculoskeletal discomfort:0.77), (CLINDAMYCINCLINDAMYCIN PHOSPHATE:Musculoskeletal discomfort:0), (DALFAMPRIDINE:Musculoskeletal discomfort:0.25), (DILTIAZEM HYDROCHLORIDE:Musculoskeletal discomfort:1.3), (DIPHENHYDRAMINE:Musculoskeletal discomfort:0.16), (DUTASTERIDE:Musculoskeletal discomfort:0.04), (ESZOPICLONE:Musculoskeletal discomfort:0.2), (EVOLOCUMAB:Musculoskeletal discomfort:0.77), (FERROUS SULFATE:Musculoskeletal discomfort:0.29), (FERROUS SULFATEFERROUS SULFATE, DRIED:Musculoskeletal discomfort:0.41), (GLIPIZIDE:Musculoskeletal discomfort:0.3), (HYDROCHLOROTHIAZIDELISINOPRIL:Musculoskeletal discomfort:0.36), (HYDROXYCHLOROQUINE DIPHOSPHATE:Musculoskeletal discomfort:0.18), (HYOSCYAMINE:Musculoskeletal discomfort:0.11), (ILOPROST:Musculoskeletal discomfort:0), (LISDEXAMFETAMINE DIMESYLATE:Musculoskeletal discomfort:0.37), (METHYLPREDNISOLONE SODIUM SUCCINATE:Musculoskeletal discomfort:0.01), (OMEGA-3-ACID ETHYL ESTERS:Musculoskeletal discomfort:0.03), (PAROXETINE:Musculoskeletal discomfort:0.31), (POLYETHYLENE GLYCOLS:Musculoskeletal discomfort:0.27), (PRAMIPEXOLE:Musculoskeletal discomfort:0.13), (PRAMIPEXOLE DIHYDROCHLORIDE:Musculoskeletal discomfort:0.35), (PRIMIDONE:Musculoskeletal discomfort:0.33), (RALOXIFENE HYDROCHLORIDE:Musculoskeletal discomfort:0.29), (SUMATRIPTANSUMATRIPTAN SUCCINATE:Musculoskeletal discomfort:0.29), (TRIAMTERENE:Musculoskeletal discomfort:0.18), (UMECLIDINIUM BROMIDEVILANTEROL TRIFENATATE:Musculoskeletal discomfort:0.4), (VITAMIN B:Musculoskeletal discomfort:0.36), (DICLOFENAC:Musculoskeletal disorder:0.14), (FINGOLIMOD HYDROCHLORIDE:Musculoskeletal disorder:0.19), (TRIAMCINOLONE ACETONIDE:Musculoskeletal disorder:0.09), (.ALPHA.1-PROTEINASE INHIBITOR HUMAN:Musculoskeletal pain:0.52), (ACTIVATED CHARCOAL:Musculoskeletal pain:0.08), (AGALSIDASE BETA:Musculoskeletal pain:0.53), (ALBIGLUTIDE:Musculoskeletal pain:0.14), (ALDESLEUKIN:Musculoskeletal pain:0.05), (ALENDRONIC ACID:Musculoskeletal pain:1), (AMANTADINE HYDROCHLORIDE:Musculoskeletal pain:0.29), (AMILORIDE HYDROCHLORIDEHYDROCHLOROTHIAZIDE:Musculoskeletal pain:0.4), (AMINO ACIDSDEXTROSEELECTROLYTES NOS:Musculoskeletal pain:0.19), (AMISULPRIDE:Musculoskeletal pain:0.63), (AMLODIPINE BESYLATEHYDROCHLOROTHIAZIDEVALSARTAN:Musculoskeletal pain:0.11), (AMLODIPINEVALSARTAN:Musculoskeletal pain:0.35), (AMMONIUM LACTATE:Musculoskeletal pain:0.19), (AMPICILLIN SODIUMSULBACTAM SODIUM:Musculoskeletal pain:0.15), (ANTIHISTAMINES NOS:Musculoskeletal pain:0.18), (ATAZANAVIR:Musculoskeletal pain:0.24), (ATOVAQUONE:Musculoskeletal pain:0.16), (AZILSARTAN KAMEDOXOMIL:Musculoskeletal pain:0.61), (BENAZEPRILBENAZEPRIL HYDROCHLORIDE:Musculoskeletal pain:0.87), (BETAMETHASONE DIPROPIONATECALCIPOTRIENECALCIPOTRIENE HYDRATE:Musculoskeletal pain:0.07), (BETAMETHASONECALCIPOTRIENE:Musculoskeletal pain:0.21), (BLINATUMOMAB:Musculoskeletal pain:0.61), (BOCEPREVIR:Musculoskeletal pain:0.1), (BUDESONIDEFORMOTEROL FUMARATE:Musculoskeletal pain:0.59), (CABOZANTINIB S-MALATE:Musculoskeletal pain:0.1), (CALCIPOTRIENE:Musculoskeletal pain:0.33), (CALCITONIN:Musculoskeletal pain:0.37), (CALCIUM\MAGNESIUM\ZINC:Musculoskeletal pain:0.05), (CANAGLIFLOZINMETFORMIN HYDROCHLORIDE:Musculoskeletal pain:0.78), (CARVEDILOL PHOSPHATE:Musculoskeletal pain:1.29), (CEFOTAXIME SODIUM:Musculoskeletal pain:0.8), (CEFUROXIME AXETIL:Musculoskeletal pain:0.23), (CETUXIMAB:Musculoskeletal pain:0.75), (CHLORAMBUCIL:Musculoskeletal pain:0.32), (CHLORDIAZEPOXIDE HYDROCHLORIDECLIDINIUM BROMIDE:Musculoskeletal pain:0.17), (CHLORPHENIRAMINEHYDROCODONE:Musculoskeletal pain:0.3), (CINACALCET HYDROCHLORIDE:Musculoskeletal pain:0.32), (CLINDAMYCIN PHOSPHATE:Musculoskeletal pain:0.46), (CLOMIPRAMINE HYDROCHLORIDE:Musculoskeletal pain:0.25), (CLOZAPINE:Musculoskeletal pain:1.33), (CORTICOTROPIN:Musculoskeletal pain:0.39), (CYPROHEPTADINE:Musculoskeletal pain:0.4), (DABRAFENIB MESYLATE:Musculoskeletal pain:0.15), (DALTEPARIN:Musculoskeletal pain:0.95), (DANTROLENE SODIUM:Musculoskeletal pain:0.01), (DARUNAVIRRITONAVIR:Musculoskeletal pain:0.25), (DASATINIB:Musculoskeletal pain:2.35), (DESONIDE:Musculoskeletal pain:0.52), (DEVICE:Musculoskeletal pain:0.42), (DIGITOXIN:Musculoskeletal pain:0.34), (DOLASETRON MESYLATE:Musculoskeletal pain:0.33), (DORZOLAMIDE:Musculoskeletal pain:0.07), (DOXEPIN:Musculoskeletal pain:0.83), (DOXYLAMINE SUCCINATE:Musculoskeletal pain:0.19), (DULAGLUTIDE:Musculoskeletal pain:0.08), (DUTASTERIDETAMSULOSIN HYDROCHLORIDE:Musculoskeletal pain:0.2), (ECONAZOLE:Musculoskeletal pain:0.55), (EDOXABAN TOSYLATE:Musculoskeletal pain:1.25), (ELOTUZUMAB:Musculoskeletal pain:0.4), (ELTROMBOPAG OLAMINE:Musculoskeletal pain:0.47), (EMPAGLIFLOZINLINAGLIPTIN:Musculoskeletal pain:0.71), (EPIRUBICIN:Musculoskeletal pain:0.74), (ERIBULIN:Musculoskeletal pain:0.19), (ESTROGENS, CONJUGATEDMEDROXYPROGESTERONE ACETATE:Musculoskeletal pain:0.87), (EVOLOCUMAB:Musculoskeletal pain:1.54), (FELODIPINE:Musculoskeletal pain:0.31), (FENOFIBRIC ACID:Musculoskeletal pain:0.39), (FENOTEROL HYDROBROMIDEIPRATROPIUM BROMIDE:Musculoskeletal pain:0.03), (FLUVOXAMINE MALEATE:Musculoskeletal pain:0.31), (FORMOTEROL FUMARATE:Musculoskeletal pain:1.01), (FULVESTRANT:Musculoskeletal pain:0.96), (GADOVERSETAMIDE:Musculoskeletal pain:0.09), (GEFITINIB:Musculoskeletal pain:0.4), (GIMERACILOTERACILTEGAFUR:Musculoskeletal pain:0.66), (GLYCOPYRROLATE:Musculoskeletal pain:0.39), (HEPARIN CALCIUM:Musculoskeletal pain:0.21), (HERBALSSAW PALMETTO:Musculoskeletal pain:0.05), (HYDROCORTISONE ACETATE:Musculoskeletal pain:0.35), (HYDROXYCHLOROQUINE DIPHOSPHATE:Musculoskeletal pain:0.65), (HYDROXYUREA:Musculoskeletal pain:0.82), (IBRITUMOMAB TIUXETAN:Musculoskeletal pain:0.06), (ILOPROST:Musculoskeletal pain:0.89), (INDACATEROL:Musculoskeletal pain:0.27), (INDAPAMIDEPERINDOPRIL:Musculoskeletal pain:1.19), (INSULIN DEGLUDEC:Musculoskeletal pain:0.06), (INTERFERON:Musculoskeletal pain:0.06), (INTERFERON ALFA-2A:Musculoskeletal pain:0.57), (INTERFERON GAMMA-1B:Musculoskeletal pain:0.21), (IRINOTECAN HYDROCHLORIDE:Musculoskeletal pain:0.72), (IRON SUCROSE:Musculoskeletal pain:0.07), (LABETALOL HYDROCHLORIDE:Musculoskeletal pain:0.66), (LACTASE:Musculoskeletal pain:0.45), (LENVATINIB:Musculoskeletal pain:0.19), (LEUCOVORIN CALCIUM:Musculoskeletal pain:0.75), (LEVOLEUCOVORIN CALCIUM:Musculoskeletal pain:0.17), (MARAVIROC:Musculoskeletal pain:0.02), (MEDROXYPROGESTERONE:Musculoskeletal pain:0.07), (MEFENAMIC ACID:Musculoskeletal pain:0.26), (MEPOLIZUMAB:Musculoskeletal pain:0.37), (MERCAPTOPURINE:Musculoskeletal pain:0.24), (METFORMIN HYDROCHLORIDEVILDAGLIPTIN:Musculoskeletal pain:0.43), (METHENAMINE HIPPURATE:Musculoskeletal pain:1.33), (METHIMAZOLE:Musculoskeletal pain:0.56), (MIDODRINE HYDROCHLORIDE:Musculoskeletal pain:0.19), (MOSAPRIDE CITRATE:Musculoskeletal pain:0.12), (MOXIFLOXACIN HYDROCHLORIDE:Musculoskeletal pain:2.43), (MOXONIDINE:Musculoskeletal pain:0.33), (NADROPARIN CALCIUM:Musculoskeletal pain:0.72), (NATEGLINIDE:Musculoskeletal pain:0.4), (NINTEDANIB:Musculoskeletal pain:1.04), (NORETHINDRONE:Musculoskeletal pain:0.73), (NORTRIPTYLINE HYDROCHLORIDE:Musculoskeletal pain:0.43), (OLMESARTAN:Musculoskeletal pain:0.03), (OLOPATADINE HYDROCHLORIDE:Musculoskeletal pain:0.01), (OMEPRAZOLE SODIUM:Musculoskeletal pain:0.42), (PATIROMER:Musculoskeletal pain:0.29), (PEGINTERFERON BETA-1A:Musculoskeletal pain:0.95), (PEMETREXED:Musculoskeletal pain:0.75), (PENTOSAN POLYSULFATE SODIUM:Musculoskeletal pain:0.49), (PHENYTOIN SODIUM:Musculoskeletal pain:0.67), (PILOCARPINE:Musculoskeletal pain:0.13), (PIMECROLIMUS:Musculoskeletal pain:0.16), (RADIATION THERAPY:Musculoskeletal pain:0.97), (RALOXIFENE HYDROCHLORIDE:Musculoskeletal pain:0.92), (RANOLAZINE:Musculoskeletal pain:1.77), (RIOCIGUAT:Musculoskeletal pain:0.14), (SALMETEROL XINAFOATE:Musculoskeletal pain:0.18), (SELEXIPAG:Musculoskeletal pain:0.01), (SILVER SULFADIAZINE:Musculoskeletal pain:0.06), (SITAGLIPTIN:Musculoskeletal pain:0.87), (SODIUM PHOSPHATE, DIBASIC\SODIUM PHOSPHATE, MONOBASIC:Musculoskeletal pain:0.06), (SULINDAC:Musculoskeletal pain:0.04), (TAMOXIFEN CITRATE:Musculoskeletal pain:0.14), (TELOTRISTAT ETHYL:Musculoskeletal pain:0.11), (TETRACYCLINE:Musculoskeletal pain:1.15), (THALIDOMIDE:Musculoskeletal pain:1.93), (TIMOLOL MALEATE:Musculoskeletal pain:0.23), (TINZAPARIN:Musculoskeletal pain:0.33), (TRAMETINIB DIMETHYL SULFOXIDE:Musculoskeletal pain:0.03), (TRANDOLAPRIL:Musculoskeletal pain:0.09), (VORTIOXETINE HYDROBROMIDE:Musculoskeletal pain:0.09), (AMIODARONE:Musculoskeletal stiffness:0.46), (AMPHETAMINE ASPARTATEAMPHETAMINE SULFATEDEXTROAMPHETAMINE SACCHARATEDEXTROAMPHETAMINE SULFATE:Musculoskeletal stiffness:1.09), (ANASTROZOLE:Musculoskeletal stiffness:0.63), (BISOPROLOL FUMARATE:Musculoskeletal stiffness:0.14), (BUPRENORPHINE HYDROCHLORIDE:Musculoskeletal stiffness:0.45), (BUSPIRONE HYDROCHLORIDE:Musculoskeletal stiffness:1.11), (CALCIUM ACETATE:Musculoskeletal stiffness:0.02), (CANDESARTAN:Musculoskeletal stiffness:0.55), (CARVEDILOL PHOSPHATE:Musculoskeletal stiffness:0.17), (CHOLESTYRAMINE:Musculoskeletal stiffness:0.52), (CIMETIDINE:Musculoskeletal stiffness:0.15), (CIPROFLOXACIN HYDROCHLORIDE:Musculoskeletal stiffness:0.56), (CLOZAPINE:Musculoskeletal stiffness:0.71), (CODEINE:Musculoskeletal stiffness:0.16), (CORTISONEHYDROCORTISONE:Musculoskeletal stiffness:0.52), (DASATINIB:Musculoskeletal stiffness:0.11), (DESLORATADINE:Musculoskeletal stiffness:0.27), (DEXLANSOPRAZOLE:Musculoskeletal stiffness:1.3), (DUTASTERIDE:Musculoskeletal stiffness:0.41), (ELETRIPTAN HYDROBROMIDE:Musculoskeletal stiffness:0.66), (EVOLOCUMAB:Musculoskeletal stiffness:0.79), (FESOTERODINE FUMARATE:Musculoskeletal stiffness:0.08), (HEPARIN SODIUM:Musculoskeletal stiffness:0.45), (HERBALSTURMERIC:Musculoskeletal stiffness:0.07), (HYDROXYCHLOROQUINE DIPHOSPHATE:Musculoskeletal stiffness:0.12), (HYDROXYZINE PAMOATE:Musculoskeletal stiffness:1.17), (IBRUTINIB:Musculoskeletal stiffness:0.97), (INDAPAMIDE:Musculoskeletal stiffness:0.25), (INSULIN HUMAN:Musculoskeletal stiffness:0.1), (ISOSORBIDE:Musculoskeletal stiffness:0.21), (ISOSORBIDE MONONITRATE:Musculoskeletal stiffness:0.49), (LABETALOLLABETALOL HYDROCHLORIDE:Musculoskeletal stiffness:0.36), (LATANOPROST:Musculoskeletal stiffness:0.01), (LEDIPASVIRSOFOSBUVIR:Musculoskeletal stiffness:0.05), (LEVALBUTEROL HYDROCHLORIDE:Musculoskeletal stiffness:0.44), (LINACLOTIDE:Musculoskeletal stiffness:0.09), (LISDEXAMFETAMINE DIMESYLATE:Musculoskeletal stiffness:0.62), (METHENAMINE HIPPURATE:Musculoskeletal stiffness:0.1), (METHYLPREDNISOLONE ACETATE:Musculoskeletal stiffness:0.27), (METRONIDAZOLE:Musculoskeletal stiffness:1.18), (NEBIVOLOL:Musculoskeletal stiffness:0.01), (NILOTINIB:Musculoskeletal stiffness:0.26), (PANTOPRAZOLE MAGNESIUM:Musculoskeletal stiffness:0.27), (PEGINTERFERON BETA-1A:Musculoskeletal stiffness:0.25), (PENICILLIN:Musculoskeletal stiffness:0.11), (PHENOBARBITAL:Musculoskeletal stiffness:0.18), (PRAMIPEXOLE DIHYDROCHLORIDE:Musculoskeletal stiffness:0.84), (PRIMIDONE:Musculoskeletal stiffness:0.63), (RABEPRAZOLE:Musculoskeletal stiffness:0.23), (RANOLAZINE:Musculoskeletal stiffness:0.56), (ROFLUMILAST:Musculoskeletal stiffness:0.01), (SACUBITRILVALSARTAN:Musculoskeletal stiffness:0.63), (SOTALOL:Musculoskeletal stiffness:0.16), (THALIDOMIDE:Musculoskeletal stiffness:0.28), (TORSEMIDE:Musculoskeletal stiffness:0.03), (TRIAMCINOLONE:Musculoskeletal stiffness:0), (.ALPHA.1-PROTEINASE INHIBITOR HUMAN:Myalgia:0.75), (ACETAMINOPHEN\CAFFEINE:Myalgia:0.05), (AGALSIDASE BETA:Myalgia:0.74), (AMILORIDE HYDROCHLORIDEHYDROCHLOROTHIAZIDE:Myalgia:0.51), (AMINO ACIDSDEXTROSEELECTROLYTES NOS:Myalgia:0.35), (AMLODIPINEPERINDOPRIL:Myalgia:0.05), (AMMONIUM LACTATE:Myalgia:0.55), (AMOXICILLINCLARITHROMYCINESOMEPRAZOLE:Myalgia: 0.06), (AMPICILLIN SODIUMSULBACTAM SODIUM:Myalgia:0.52), (ANTIHISTAMINES NOS:Myalgia:0.3), (ARIPIPRAZOLE LAUROXIL:Myalgia:0.21), (ASUNAPREVIR:Myalgia:0.25), (ATROPINEDIPHENOXYLATE: Myalgia:0.22), (AZELNIDIPINE:Myalgia:0.1), (BECLOMETHASONEFORMOTEROL:Myalg ia:0.14), (BENZTROPINE MESYLATE:Myalgia:0.23), (BETAMETHASONE DIPROPIONATECALCIPOTRIENE:Myalgia:0.16), (BETAMETHASONE DIPROPIONATECALCIPOTRIENECALCIPOTRIENE HYDRATE:Myalgia:0.74), (BETAMETHASONECALCIPOTRIENE:Myalgia:0.53), (BETHAN ECHOL CHLORIDE:Myalgia:0.06), (BISOPROLOL FUMARATEHYDROCHLOROTHIAZIDE:Myalgia:0.13), (BLINATUMOMAB:Myalgia:0.05), (BUDESONIDEFORMOTEROL FUMARATE:Myalgia:0.95), (BUTALBITAL:Myalgia:0.01), (CABOZANTINIB S-MALATE:Myalgia:0.26), (CALCITONIN SALMON:Myalgia:0.89), (CALCIUM\MAGNESIUM:Myalgia:0.63), (CALCIUMMAGNESIU MZ1NC:Myalgia:0.91), (CANAKINUMAB:Myalgia:0.94), (CANDESARTAN CILEXETILHYDROCHLOROTHIAZIDE:Myalgia:2.09), (CAPTOPRIL:Myalgia:1.31), (CAR BIDOPA:Myalgia:0.2), (CARVEDILOL PHOSPHATE:Myalgia:1.74), (CEFUROXIME:Myalgia:0.34), (CETUXIMAB:Myalgia:0.32), (C EVIMELINE HYDROCHLORIDE:Myalgia:0.27), (CHLORHEXIDINE GLUCONATE:Myalgia:0.24), (CHLORPHENIRAMINEHYDROCODONE:Myalgia:0.43), (CI LNIDIPINE:Myalgia:0.06), (CINACALCET HYDROCHLORIDE:Myalgia:0.3), (CIPROFIBRATE:Myalgia:0.21), (CLINDAMYCIN:Myalgi a:1.73), (CLOMIPRAMINE HYDROCHLORIDE:Myalgia:0.37), (COLESTIPOL:Myalgia:0.27), (CRIZOTINIB:Myalgia:1.08), (CYPROHEPTADINE HYDROCHLORIDE:Myalgia:0.01), (DABIGATRAN:Myalgia:0.28), (DACLIZUMAB:Myalgia: 1.67), (DANAZOL:Myalgia:0.68), (DANTROLENE SODIUM:Myalgia:0.33), (DEGARELIX:Myalgia:0.04), (DIGITOXIN:Myalgia:0.11), (DILAZEP: Myalgia:0.11), (DIPYRONE:Myalgia:0.84), (DIPYRONEPROMETHAZINE HYDROCHLORIDE:Myalgia:0.12), (DOFETILIDE:Myalgia:0.99), (DOLASETRON MESYLATE:Myalgia:0.35), (DORZOLAMIDE HYDROCHLORIDETIMOLOL MALEATE:Myalgia:0.06), (DULAGLUTIDE:Myalgia:0.34), (ECONAZOLE:Myalgia:0.67), (ED OXABAN TOSYLATE:Myalgia:1.3), (EFINACONAZOLE:Myalgia:0.13), (ELBASVIRGRAZOPREVIR: Myalgia:0.15), (EMPAGLIFLOZINLINAGLIPTIN:Myalgia:1.01), (ERIBULIN:Myalgia:0.1), (E THINYL ESTRADIOLNORGESTREL:Myalgia:0.17), (ETRETINATE:Myalgia:0.59), (FENOTEROL HYDROBROMIDEIPRATROPIUM BROMIDE:Myalgia:0.05), (FLUINDIONE:Myalgia:0.06), (FLUOCINOLONE ACETONIDE:Myalgia:0.02), (FLURAZEPAMFLURAZEPAM HYDROCHLORIDE:Myalgia:0.11), (FLUTICASONE FUROATESALMETEROL XINAFOATE:Myalgia:1.02), (FLUVOXAMINE MALEATE:Myalgia:0.83), (FONDAPARINUX SODIUM:Myalgia:0.18), (FORMOTEROL:Myalgia:0.85), (FORMOTEROL FUMARATE:Myalgia:1.05), (FORMOTEROL FUMARATE\MOMETASONE FUROATE:Myalgia:0.46), (GEFITINIB:Myalgia:0.29), (GENTAMICIN:Myalgia:0.34), (GIMER ACILOTERACILTEGAFUR:Myalgia:0.68), (GLIPIZIDEMETFORMIN HYDROCHLORIDE:Myalgia:0.01), (GLYCOPYRROLATE:Myalgia:0.58), (GLYCOPYRROL ATEINDACATEROL MALEATE:Myalgia:0.12), (GLYCOPYRRONIUM\INDACATEROL:Myalgia:0.94), (GOSERE LIN ACETATE:Myalgia:0.08), (GUANFACINE HYDROCHLORIDE:Myalgia:0.45), (HEPARIN CALCIUM:Myalgia:0.1), (HERBALSSAW PALMETTO:Myalgia:0.12), (HYDROCHLOROTHIAZIDEIRBESARTAN:Myalgia:1.64), (IL OPROST:Myalgia:0.8), (IMIPRAMINE:Myalgia:0.31), (IMMUNE GLOBULIN NOS:Myalgia:0.17), (INDACATEROL:Myalgia:0.23), (INFLIXIMAB-DYYB:Myalgia:0.57), (INSULIN BEEF:Myalgia:0.28), (INSULIN DEGLUDEC:Myalgia:0.29), (INTERFERON:Myalgia:0.45), (INTERFERON ALFA-2A:Myalgia:0.83), (INTERFERON ALFA-2B:Myalgia:0.67), (INTERFERON GAMMA-1B:Myalgia:0.11), (IRINOTECAN HYDROCHLORIDE:Myalgia:0.43), (ISONIAZID:Myalgia:0.54), (ITRACONAZOLE:Myalgia:00.71), (LANREOTIDE:Myalgia:0.09), (LANREOTIDE ACETATE:Myalgia:0.03), (MAGNESIUM GLUCONATE:Myalgia:0.01), (MESNA:Myalgia:0.4), (METHENAMINE HIPPURATE:Myalgia:1.65), (MIDODRINE HYDROCHLORIDE:Myalgia:0.46), (MORPHINE SULFATE\NALTREXONE HYDROCHLORIDE:Myalgia:0.67), (NADOLOL:Myalgia:1.15), (NICARDIPINE:Myalgia:0.2), (NINTEDANIB:Myalgia:0.93), (OCTREOTIDE:Myalgia:0.02), (OLMESARTAN:Myalgia:0.09), (OSELTAMIVIR:Myalgia:0.11), (OSPEMIFENE:Myalgia:0.12), (OXYBUTYNINOXYBUTYN IN CHLORIDE:Myalgia:0.37), (PATIROMER:Myalgia:0.29), (PAZOPANIB HYDROCHLORIDE:Myalgia:1.79), (PEMETREXED:Myalgia:0.73), (PEMETREXED DISODIUM:Myalgia:1.08), (PENICILLIN G:Myalgia:0.24), (PILOCARPINE HYDROCHLORIDE:Myalgia:0.1), (PILSICAINIDE HYDROCHLORIDE:Myalgia:0.08), (PIOGLITAZONE:Myalgia:0.31), (PNEUMOCOCCAL VACCINE, POLYVALENT 23:Myalgia:0.27), (POLYETHYLENE GLYCOL 4000:Myalgia:0.04), (PRANLUKAST:Myalgia:0.16), (PREDNISOLONE SODIUM PHOSPHATE:Myalgia:0.19), (PROTRIPTYLINE HYDROCHLORIDE:Myalgia:0.37), (QUINACRINE:Myalgia:0.31), (RADIATION THERAPY:Myalgia:0.83), (RASBURICASE:Myalgia:0.04), (REPAGLINIDE:Myalgia:0.04), (R ETINOL:Myalgia:0.07), (SCOPOLAMINE:Myalgia:1.52), (SITAGLIPTIN:Myalgia:1.19), (SOF OSBUVIRVELPATASVIR:Myalgia:0.14), (STARCH, WHEAT:Myalgia:0.22), (SULFAMETHOXAZOLE:Myalgia:1.15), (TAMOXIFEN CITRATE:Myalgia:0.18), (TELOTRISTAT ETHYL:Myalgia:0.1), (TINZAPARIN:Myalgia:0.3), (TRAMETINIB DIMETHYL SULFOXIDE:Myalgia:0.14), (TRIPTORELIN PAMOATE:Myalgia:0.23), (UMECLIDINIUM BROMIDE:Myalgia:0.76), (UMECLIDINIUM BROMIDEVILANTEROL TRIFENATATE:Myalgia:1.15), (URAPIDIL:Myalgia:0.01), (YEAST:Myalgia:0.05), (ZILEUTO N:Myalgia:0.39), (BISACODYL OR DOCUSATE SODIUM:Myasthenia gravis:0.5), (BORTEZOMIB:Myasthenia gravis:0.22), (BROMAZEPAM:Myasthenia gravis:0), (CALCITRIOL:Myasthenia gravis:0.53), (CALCIUM ACETATE:Myasthenia gravis:0.05), (CALCIUM CITRATE:Myasthenia gravis:0.39), (CANDESARTAN CILEXETILHYDROCHLOROTHIAZIDE:Myasthenia gravis:0.27), (CARFILZOMIB:Myasthenia gravis:0.16), (CARVEDILOL PHOSPHATE:Myasthenia gravis:0.28), (CERTOLIZUMAB PEGOL:Myasthenia gravis:0.89), (CIPROFLOXACIN HYDROCHLORIDE:Myasthenia gravis:0.29), (CLINDAMYCINCLINDAMYCIN PHOSPHATE:Myasthenia gravis:0.48), (CLOZAPINE:Myasthenia gravis:0.09), (DACLIZUMAB:Myasthenia gravis:0.3), (DALFAMPRIDINE:Myasthenia gravis:0.01), (DAPAGLIFLOZIN PROPANEDIOL:Myasthenia gravis:0.04), (DASATINIB:Myasthenia gravis:0.46), (DESLORATADINE:Myasthenia gravis:0.14), (DEVICE:Myasthenia gravis:0.54), (DUTASTERIDE:Myasthenia gravis:0.45), (ESZOPICLONE:Myasthenia gravis:0.72), (EVEROLIMUS:Myasthenia gravis:0.55), (EVOLOCUMAB:Myasthenia gravis:0.99), (FESOTERODINE FUMARATE:Myasthenia gravis:0.16), (FLUOXETINEFLUOXETINE HYDROCHLORIDE:Myasthenia gravis:0.11), (HYDROCHLOROTHIAZIDEIRBESARTAN:Myasthenia gravis:0.24), (HYDROXYZINE PAMOATE:Myasthenia gravis:0.49), (IBRUTINIB:Myasthenia gravis:1.14), (ILOPROST:Myasthenia gravis:0.37), (IMIPRAMINE HYDROCHLORIDE:Myasthenia gravis:0.19), (IPRATROPIUM:Myasthenia gravis:0.26), (LERCANIDIPINE:Myasthenia gravis:0.12), (METHOCARBAMOL:Myasthenia gravis:0.17), (METHYLPREDNISOLONE ACETATE:Myasthenia gravis:0.04), (METOLAZONE:Myasthenia gravis:0.08), (MOXIFLOXACIN HYDROCHLORIDE:Myasthenia gravis:0.2), (NEBIVOLOL HYDROCHLORIDE:Myasthenia gravis:0.32), (NILOTINIB:Myasthenia gravis:0.29), (NITROFURANTOIN:Myasthenia gravis:0.39), (NORTRIPTYLINE:Myasthenia gravis:0.2), (OLANZAPINE:Myasthenia gravis:0.64), (OXYBUTYNIN CHLORIDE:Myasthenia gravis:0.13), (PANTOPRAZOLE MAGNESIUM:Myasthenia gravis:0.22), (PAZOPANIB HYDROCHLORIDE:Myasthenia gravis:0.14), (RANOLAZINE:Myasthenia gravis:1.24), (RIZATRIPTAN BENZOATE:Myasthenia gravis:0.12), (ROFLUMILAST:Myasthenia gravis:0.07), (SECUKINUMAB:Myasthenia gravis:0.16), (SOTALOL:Myasthenia gravis:0.49), (SUMATRIPTANSUMATRIPTAN SUCCINATE:Myasthenia gravis:0), (TERIFLUNOMIDE:Myasthenia gravis:0.47), (TICAGRELOR:Myasthenia gravis:0.01), (TIZANIDINE HYDROCHLORIDE:Myasthenia gravis:0.77), (TOCILIZUMAB:Myasthenia gravis:0.19), (TRIAMCINOLONE ACETONIDE:Myasthenia gravis:0.45), (ACARBOSE:Mycosis fungoides:0.16), (ACETAZOLAMIDE:Mycosis fungoides:0.09), (ALISKIREN HEMIFUMARATE:Mycosis fungoides:0.01), (AMITRIPTYLINE:Mycosis fungoides:0.81), (AMITRIPTYLINE HYDROCHLORIDE:Mycosis fungoides:0.13), (ARMODAFINIL:Mycosis fungoides:0.39), (BISACODYL:Mycosis fungoides:0.23), (CALCIUM ACETATE:Mycosis fungoides:0.46), (CALCIUM CARBONATECHOLECALCIFEROL:Mycosis fungoides:0.95), (CANDESARTAN CILEXETIL:Mycosis fungoides:0.92), (CARISOPRODOL:Mycosis fungoides:0.18), (CARVEDILOL PHOSPHATE:Mycosis fungoides:0.32), (CERTOLIZUMAB PEGOL:Mycosis fungoides:0.88), (CHOLESTYRAMINE:Mycosis fungoides:0.83), (CIMETIDINE:Mycosis fungoides:0.07), (CINACALCET HYDROCHLORIDE:Mycosis fungoides:0.04), (DACLIZUMAB:Mycosis fungoides:0.33), (DALFAMPRIDINE:Mycosis fungoides:0.27), (DAPAGLIFLOZIN PROPANEDIOL:Mycosis fungoides:0.22), (DASATINIB:Mycosis fungoides:0.75), (DESLORATADINE:Mycosis fungoides:0.29), (DESVENLAFAXINE SUCCINATE:Mycosis fungoides:0.01), (DEVICE:Mycosis fungoides:0.03), (DOFETILIDE:Mycosis fungoides:0.17), (EDOXABAN TOSYLATE:Mycosis fungoides:0.21), (ELETRIPTAN HYDROBROMIDE:Mycosis fungoides:0.33), (ESTROGENS, CONJUGATEDMEDROXYPROGESTERONE ACETATE:Mycosis fungoides:0.24), (EVEROLIMUS:Mycosis fungoides:0.47), (EVOLOCUMAB:Mycosis fungoides:0.72), (FESOTERODINE FUMARATE:Mycosis fungoides:0.38), (FINGOLIMOD HYDROCHLORIDE:Mycosis fungoides:0.03), (HYDROCHLOROTHIAZIDEIRBESARTAN:Mycosis fungoides:0.27), (HYDROMORPHONE:Mycosis fungoides:0.41), (HYDROXYCHLOROQUINE DIPHOSPHATE:Mycosis fungoides:0.01), (HYDROXYZINE PAMOATE:Mycosis fungoides:0.61), (IDELALISIB:Mycosis fungoides:0.51), (ILOPROST:Mycosis fungoides:0.07), (INDAPAMIDE:Mycosis fungoides:0.06), (INFLUENZA VIRUS VACCINE:Mycosis fungoides:0.08), (INSULIN NOS:Mycosis fungoides:0.59), (IPRATROPIUM:Mycosis fungoides:0.49), (ISOSORBIDE MONONITRATE:Mycosis fungoides:1.12), (LACTASE:Mycosis fungoides:0.06), (LEDIPASVIRSOFOSBUVIR:Mycosis fungoides:0.46), (LERCANIDIPINE:Mycosis fungoides:0.16), (METHENAMINE HIPPURATE:Mycosis fungoides:0.08), (METHYLPREDNISOLONE ACETATE:Mycosis fungoides:0.25), (METRONIDAZOLEMETRONIDAZOLE HYDROCHLORIDE:Mycosis fungoides:0.53), (MINOCYCLINEMINOCYCLINE HYDROCHLORIDE:Mycosis fungoides:0.37), (MODAFINIL:Mycosis fungoides:0.38), (MOXIFLOXACIN:Mycosis fungoides:0.06), (NALOXONE HYDROCHLORIDEOXYCODONE HYDROCHLORIDE:Mycosis fungoides:0.3), (NATEGLINIDE:Mycosis fungoides:0.01), (NEBIVOLOL:Mycosis fungoides:0.32), (NILOTINIB:Mycosis fungoides:0.68), (PANTOPRAZOLE MAGNESIUM:Mycosis fungoides:0.39), (PAZOPANIB HYDROCHLORIDE:Mycosis fungoides:0.56), (PENICILLIN:Mycosis fungoides:0.48), (PONATINIB:Mycosis fungoides:0.03), (PRAMIPEXOLE:Mycosis fungoides:0.15), (PREDNISOLONE ACETATE:Mycosis fungoides:0.09), (QUETIAPINE:Mycosis fungoides:0.45), (RIZATRIPTAN BENZOATE:Mycosis fungoides:0.25), (ROPINIROLE HYDROCHLORIDE:Mycosis fungoides:0.03), (SACUBITRILVALSARTAN:Mycosis fungoides:0.39), (SECUKINUMAB:Mycosis fungoides:0.47), (SODIUM OXYBATE:Mycosis fungoides:0.87), (SUMATRIPTAN:Mycosis fungoides:0.09), (SUMATRIPTANSUMATRIPTAN SUCCINATE:Mycosis fungoides:0.29), (SUNITINIB MALATE:Mycosis fungoides:0.03), (TERIFLUNOMIDE:Mycosis fungoides:0.48), (TETRACYCLINE:Mycosis fungoides:0.56), (TOFACITINIB CITRATE:Mycosis fungoides:0.26), (UMECLIDINIUM BROMIDEVILANTEROL TRIFENATATE:Mycosis fungoides:0.32), (VALGANCICLOVIR HYDROCHLORIDE:Mycosis fungoides:0.06), (VARENICLINE TARTRATE:Mycosis fungoides:0.79), (TETRACAINE:Mydriasis:0.83), (ACETAMINOPHENAPRONALIDECAFF EINEPROPYPHENAZONE:Myelodysplastic syndrome:0.26), (ACETAMINOPHEN\CAFFEINE:Myelodysplastic syndrome:0.43), (ACETAMINOPHENPROPOXYPHENE HYDROCHLORIDE:Myelodysplastic syndrome:0.62), (ACETAMINOPHENPROPOXYPHENE NAPSYLATE:Myelodysplastic syndrome:0.78), (ACTIVATED CHARCOAL:Myelodysplastic syndrome:1.23), (ADENINE:Myelodysplastic syndrome:0.42), (ADO-TRASTUZUMAB EMTANSINE:Myelodysplastic syndrome:0.83), (AFATINIB:Myelodysplastic syndrome:0.01), (AGALSIDASE BETA:Myelodysplastic syndrome:1.01), (AMANTADINE:Myelodysplastic syndrome:0.91), (AMANTADINE HYDROCHLORIDE:Myelodysplastic syndrome:0.45), (AMBROXOL:Myelodysplastic syndrome:0.03), (AMINO ACIDSCARBOHYDRATESMINERALSSOYBEAN OILVITAMINS:Myelodysplastic syndrome:0.66), (AMISULPRIDE:Myelodysplastic syndrome:0.65), (AMLODIPINE BESYLATEHYDROCHLOROTHIAZIDEOLMESARTAN MEDOXOMIL:Myelodysplastic syndrome:0.64), (AMMONIUM GLYCYRRHIZATECYSTEINE HYDROCHLORIDEGLYCINE:Myelodysplastic syndrome:0.76), (AMOXAPINE:Myelodysplastic syndrome:0.56), (ANTIHISTAMINES NOS:Myelodysplastic syndrome:0.47), (ARMODAFINIL:Myelodysplastic syndrome:1.79), (ARSENIC TRIOXIDE:Myelodysplastic syndrome:0.1), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Myelodysplastic syndrome:0.15), (ASCORBIC ACIDCALCIUM PANTOTHENATE:Myelodysplastic syndrome:0.09), (ASPARAGINASE:Myelodysplastic syndrome:0.96), (ATENOLOLCHLORTHALIDONE:Myelodysplastic syndrome:0.13), (AZELASTINE HYDROCHLORIDEFLUTICASONE PROPIONATE:Myelodysplastic syndrome:0.21), (AZELNIDIPINEOLMESARTAN MEDOXOMIL:Myelodysplastic syndrome:0.59), (BACITRACIN:Myelodysplastic syndrome:1.23), (BALSALAZIDE DISODIUM:Myelodysplastic syndrome:0.49), (BENIDIPINE:Myelodysplastic syndrome:0.41), (BETAMETHASONE DIPROPIONATECALCIPOTRIENECALCIPOTRIENE HYDRATE:Myelodysplastic syndrome:0.64), (BETAMETHASONE SODIUM PHOSPHATENEOMYCIN SULFATE:Myelodysplastic syndrome:0.4), (BETAMETHASONECALCIPOTRIENE:Myelodysplastic syndrome:0.71), (BIXALOMER:Myelodysplastic syndrome:0.02), (BLINATUMOMAB:Myelodysplastic syndrome:2.04), (BOCOCIZUMAB:Myelodysplastic syndrome:0.14), (BOSUTINIB:Myelodysplastic syndrome:0.17), (BROMHEXINE:Myelodysplastic syndrome:0.01), (BUDESONIDEFORMOTEROL:Myelodysplastic syndrome:0.26), (BUPRENORPHINE HYDROCHLORIDENALOXONE HYDROCHLORIDE:Myelodysplastic syndrome:0.48), (CABAZITAXEL:Myelodysplastic syndrome:0.18), (CALCIUM CARBONATECHOLECALCIFEROLMAGNESIUM CARBONATE:Myelodysplastic syndrome:1.26), (CALCIUM CHLORIDEDEXTROSE\POTASSIUM CHLORIDE\SODIUM ACETATE\SODIUM CHLORIDE:Myelodysplastic syndrome:0.38), (CALCIUM\MAGNESIUM\ZINC:Myelodysplastic syndrome:0.42), (CANAGLIFLOZIN:Myelodysplastic syndrome:0.33), (CANAGLIFLOZINMETFORMIN HYDROCHLORIDE:Myelodysplastic syndrome:0.79), (CAPSAICIN:Myelodysplastic syndrome:0.02), (CARVEDILOL PHOSPHATE:Myelodysplastic syndrome:2.3), (CEFOTAXIME SODIUM:Myelodysplastic syndrome:1.34), (CEFOTAXIMECEFOTAXIME SODIUM:Myelodysplastic syndrome:0.12), (CERITINIB:Myelodysplastic syndrome:0.29), (CERTOLIZUMAB PEGOL:Myelodysplastic syndrome:1.85), (CEVIMELINE HYDROCHLORIDE:Myelodysplastic syndrome:0.38), (CHLORDIAZEPOXIDE HYDROCHLORIDECLIDINIUM BROMIDE:Myelodysplastic syndrome:0.68), (CHLOROTHIAZIDE:Myelodysplastic syndrome:0.11), (CHLORPHENIRAMINE MALEATDIHYDROCODEINE PHOSPHATEMETHYLEPHEDRINE HYDROCHLORIDE, DL-:Myelodysplastic syndrome:0.17), (CHLORPHENIRAMINEHYDROCODONE:Myelodysplastic syndrome:0.78), (CICLESONIDE:Myelodysplastic syndrome:1.08), (CLIKSTAR:Myelodysplastic syndrome:0.09), (CLOMIPRAMINE HYDROCHLORIDE:Myelodysplastic syndrome:0.57), (COLISTIMETHATE SODIUM:Myelodysplastic syndrome:0.36), (CORTICOTROPIN:Myelodysplastic syndrome:0.75), (CYCLOPHOSPHAMIDECYTARABINEDEXAMETHASONEDOXORUBI CINMETHOTREXATEVINCRISTINE:Myelodysplastic syndrome:0.21), (CYPROHEPTADINE:Myelodysplastic syndrome:1.13), (DABIGATRAN:Myelodysplastic syndrome:1.54), (DABRAFENIB:Myelodysplastic syndrome:0.03), (DACLIZUMAB:Myelodysplastic syndrome:1.15), (DALFAMPRIDINE:Myelodysplastic syndrome:0.05), (DALTEPARIN:Myelodysplastic syndrome:1.59), (DANTROLENE SODIUM:Myelodysplastic syndrome:0.15), (DAPAGLIFLOZIN PROPANEDIOL:Myelodysplastic syndrome:0.57), (DEGARELIX:Myelodysplastic syndrome:0.56), (DEQUALINIUM CHLORIDE:Myelodysplastic syndrome:0.07), (DEVICE:Myelodysplastic syndrome:0.05), (DIFLUNISAL:Myelodysplastic syndrome:0.31), (DILAZEP:Myelodysplastic syndrome:1.01), (DIMEMORFAN PHOSPHATE:Myelodysplastic syndrome:0.29), (DIPYRONEPROMETHAZINE HYDROCHLORIDE:Myelodysplastic syndrome:0.23), (DIQUAFOSOL TETRASODIUM:Myelodysplastic syndrome:0.15), (DISOPYRAMIDE:Myelodysplastic syndrome:0.14), (DOXYLAMINE SUCCINATE:Myelodysplastic syndrome:0.17), (EDARAVONE:Myelodysplastic syndrome:0.6), (EDOXABAN TOSILATE MONOHYDRATE:Myelodysplastic syndrome:0.47), (ELETRIPTAN HYDROBROMIDE:Myelodysplastic syndrome:1.81), (EMPAGLIFLOZINLINAGLIPTIN:Myelodysplastic syndrome:0.23), (EPIRUBICIN:Myelodysplastic syndrome:0.13), (ESTROGENS:Myelodysplastic syndrome:0.41), (ESTRONESUS SCROFA OVARY:Myelodysplastic syndrome:0.38), (ETHACRYNIC ACID:Myelodysplastic syndrome:0.23), (EVEROLIMUS:Myelodysplastic syndrome:1.85), (EVOLOCUMAB:Myelodysplastic syndrome:1.31), (FENOTEROL HYDROBROMIDEIPRATROPIUM BROMIDE:Myelodysplastic syndrome:0.89), (FERRIC OXYHYDROXIDE:Myelodysplastic syndrome:0.58), (FERROUS GLYCINE SULFATE:Myelodysplastic syndrome:0.08), (FLAVIN ADENINE DINUCLEOTIDE:Myelodysplastic syndrome:0.49), (FLOMOXEF:Myelodysplastic syndrome:0.03), (FORMOTEROL FUMARATE\MOMETASONE FUROATE:Myelodysplastic syndrome:0.77), (FOSFOMYCIN:Myelodysplastic syndrome:0.86), (FOSFOMYCIN CALCIUM:Myelodysplastic syndrome:0.05), (FURSULTIAMINE:Myelodysplastic syndrome:0.69), (FUSIDIC ACID:Myelodysplastic syndrome:1.37), (GARENOXACIN:Myelodysplastic syndrome:0.51), (GIMERACILOTERACILTEGAFUR:Myelodysplastic syndrome:1.07), (GLIPIZIDEMETFORMIN HYDROCHLORIDE:Myelodysplastic syndrome:0.19), (GLYCOPYRROLATEINDACATEROL MALEATE:Myelodysplastic syndrome:0.4), (GLYCOPYRRONIUMINDACATEROL:Myelodysplastic syndrome:1.64), (HYDROCODONE BITARTRATEIBUPROFEN:Myelodysplastic syndrome:0.51), (HYDROCORTISONE PROBUTATE:Myelodysplastic syndrome:0.03), (HYDROXYCHLOROQUINE DIPHOSPHATE:Myelodysplastic syndrome:0.79), (ICOSAPENT:Myelodysplastic syndrome:0.19), (IDARUCIZUMAB:Myelodysplastic syndrome:0.15), (IFENPRODIL:Myelodysplastic syndrome:0.09), (IGURATIMOD:Myelodysplastic syndrome:1.03), (IMIGLUCERASE:Myelodysplastic syndrome:0.02), (IMMUNE GLOBULIN NOS:Myelodysplastic syndrome:0.67), (INDACATEROL:Myelodysplastic syndrome:0.89), (INOTUZUMAB OZOGAMICIN:Myelodysplastic syndrome:1.23), (INSULIN DEGLUDEC:Myelodysplastic syndrome:0.04), (INTERFERON:Myelodysplastic syndrome:0.7), (INTERFERON ALFA-2A:Myelodysplastic syndrome:0.68), (INTERFERON GAMMA-1B:Myelodysplastic syndrome:0.88), (IPRAGLIFLOZIN L-PROLINE:Myelodysplastic syndrome:0.13), (IRINOTECAN:Myelodysplastic syndrome:0.12), (IRINOTECAN HYDROCHLORIDE:Myelodysplastic syndrome:0.49), (ITOPRIDE:Myelodysplastic syndrome:0.12), (KETOROLACKETOROLAC TROMETHAMINE:Myelodysplastic syndrome:0.91), (LANDIOLOL HYDROCHLORIDE:Myelodysplastic syndrome:0.39), (LAPATINIB DITOSYLATE:Myelodysplastic syndrome:0.24), (LENVATINIB:Myelodysplastic syndrome:0.02), (LEVOCABASTINE HYDROCHLORIDE:Myelodysplastic syndrome:0.5), (LOMITAPIDE MESYLATE:Myelodysplastic syndrome:0.67), (LYSOZYME HYDROCHLORIDE:Myelodysplastic syndrome:0.7), (MAGNESIUM ASPARTATE HYDROCHLORIDE:Myelodysplastic syndrome:0), (MAGNESIUM ASPARTATE\POTASSIUM ASPARTATE:Myelodysplastic syndrome:0.16), (MAGNESIUM GLUCONATE:Myelodysplastic syndrome:0.58), (MANIDIPINE HYDROCHLORIDE:Myelodysplastic syndrome:0), (MEPENZOLATE BROMIDE:Myelodysplastic syndrome:0.13), (MEPOLIZUMAB:Myelodysplastic syndrome:0.66), (METFORMIN HYDROCHLORIDESAXAGLIPTIN:Myelodysplastic syndrome:0.05), (METHENAMINE HIPPURATE:Myelodysplastic syndrome:1.49), (METHYLPREDNISOLONE ACETATE:Myelodysplastic syndrome:1.66), (MEXILETINE:Myelodysplastic syndrome:0.12), (MITIGLINIDE CALCIUM HYDRATEVOGLIBOSE:Myelodysplastic syndrome:0.32), (MIZORIBINE:Myelodysplastic syndrome:1.26), (MOEXIPRIL HYDROCHLORIDE:Myelodysplastic syndrome:0.47), (MORPHINE SULFATE\NALTREXONE HYDROCHLORIDE:Myelodysplastic syndrome:0.2), (NALOXONE HYDROCHLORIDETILIDINE HYDROCHLORIDE:Myelodysplastic syndrome:1.36), (NELARABINE:Myelodysplastic syndrome:0.51), (NINTEDANIB:Myelodysplastic syndrome:1.66), (OBINUTUZUMAB:Myelodysplastic syndrome:0.41), (OCTOTIAMINE:Myelodysplastic syndrome:0.15), (OFATUMUMAB:Myelodysplastic syndrome:0.28), (ORPHENADRINE:Myelodysplastic syndrome:0.3), (OXYMETAZOLINE:Myelodysplastic syndrome:0.55), (PAMIDRONATE DISODIUM:Myelodysplastic syndrome:1.38), (PATIROMER:Myelodysplastic syndrome:0.71), (PAZUFLOXACIN:Myelodysplastic syndrome:0.01), (PEGASPARGASE:Myelodysplastic syndrome:1.17), (PEGINTERFERON BETA-1A:Myelodysplastic syndrome:0.71), (PEMETREXED:Myelodysplastic syndrome:0.82), (PEMETREXED DISODIUM:Myelodysplastic syndrome:1.21), (PENTOSAN POLYSULFATE SODIUM:Myelodysplastic syndrome:0.73), (PERTUZUMAB:Myelodysplastic syndrome:0.15), (PIMECROLIMUS:Myelodysplastic syndrome:0.53), (PIMOBENDAN:Myelodysplastic syndrome:1.29), (PIRARUBICIN:Myelodysplastic syndrome:0.13), (PIRARUBICIN HYDROCHLORIDE:Myelodysplastic syndrome:0.19), (PIRFENIDONE:Myelodysplastic syndrome:0.15), (PLERIXAFOR:Myelodysplastic syndrome:0.14), (POLYMYXIN B SULFATE:Myelodysplastic syndrome:0.85), (PRAZOSIN HYDROCHLORIDE:Myelodysplastic syndrome:0.7), (PROTRIPTYLINE HYDROCHLORIDE:Myelodysplastic syndrome:0.13), (PYRAZINAMIDE:Myelodysplastic syndrome:0.74), (QUINACRINE:Myelodysplastic syndrome:0.29), (QUINAPRIL HYDROCHLORIDE:Myelodysplastic syndrome:0.02), (RAMUCIRUMAB:Myelodysplastic syndrome:0.52), (REGORAFENIB:Myelodysplastic syndrome:0.5), (RETINOL:Myelodysplastic syndrome:0.71), (SACUBITRILVALSARTAN:Myelodysplastic syndrome:0.63), (SARPOGRELATE HYDROCHLORIDE:Myelodysplastic syndrome:0.54), (SECUKINUMAB:Myelodysplastic syndrome:1.22), (SENNOSIDE B:Myelodysplastic syndrome:0.03), (SEVELAMER:Myelodysplastic syndrome:0.07), (SEVELAMER HYDROCHLORIDE:Myelodysplastic syndrome:2.15), (SIPULEUCEL-T:Myelodysplastic syndrome:0.09), (SODIUM CITRATE:Myelodysplastic syndrome:0.07), (SODIUM FERROUS CITRATE:Myelodysplastic syndrome:0.97), (SODIUM GUALENATE MONOHYDRATE:Myelodysplastic syndrome:0.35), (SODIUM OXYBATE:Myelodysplastic syndrome:1.2), (FAECALIS:Myelodysplastic syndrome:0.05), (SULFAMERAZINETRIMETHOPRIM:Myelodysplastic syndrome:0.6), (SULINDAC:Myelodysplastic syndrome:0.44), (SUNITINIB MALATE:Myelodysplastic syndrome:0.93), (SUPLATAST TOSILATE:Myelodysplastic syndrome:0.31), (TEDUGLUTIDE\WATER:Myelodysplastic syndrome:0.45), (TEMOCAPRIL HYDROCHLORIDE:Myelodysplastic syndrome:0.87), (TEMSIROLIMUS:Myelodysplastic syndrome:0.06), (TERBUTALINE:Myelodysplastic syndrome:0.73), (TERIFLUNOMIDE:Myelodysplastic syndrome:0.31), (THEOPHYLLINE:Myelodysplastic syndrome:0.51), (TIGECYCLINE:Myelodysplastic syndrome:0.96), (TIMOLOL MALEATETRAVOPROST:Myelodysplastic syndrome:0.19), (TISAGENLECLEUCEL:Myelodysplastic syndrome:0.01), (TRANDOLAPRIL:Myelodysplastic syndrome:0.72), (TRIMIPRAMINE MALEATE:Myelodysplastic syndrome:0.3), (TRIPTORELIN PAMOATE:Myelodysplastic syndrome:0), (TROXIPIDE:Myelodysplastic syndrome:0.41), (VARDENAFIL HYDROCHLORIDE TRIHYDRATE:Myelodysplastic syndrome:0.2), (VEMURAFENIB:Myelodysplastic syndrome:0.08), (VINORELBINE TARTRATE:Myelodysplastic syndrome:0.37), (VONOPRAZAN:Myelodysplastic syndrome:0.45), (YEAST:Myelodysplastic syndrome:0.3), (ZALTOPROFEN:Myelodysplastic syndrome:0.45), (ZILEUTON:Myelodysplastic syndrome:0.46), (ZINC OXIDE:Myelodysplastic syndrome:0.02), (ZOLMITRIPTAN:Myelodysplastic syndrome:0.16), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Myelofibrosis:0.11), (.ALPHA.1-PROTEINASE INHIBITOR HUMAN:Myelofibrosis:0.55), (ACETAMINOPHENPROPOXYPHENE HYDROCHLORIDE:Myelofibrosis:0.76), (ACETAMINOPHENPROPOXYPHENE NAPSYLATE:Myelofibrosis:0.01), (ADO-TRASTUZUMAB EMTANSINE:Myelofibrosis:0.11), (AGALSIDASE BETA:Myelofibrosis:1.15), (ALEMTUZUMAB:Myelofibrosis:0.05), (ALISKIREN HEMIFUMARATE:Myelofibrosis:0.82), (AMANTADINE HYDROCHLORIDE:Myelofibrosis:0.38), (AMIKACIN:Myelofibrosis:0.12), (AMINO ACIDSDEXTROSEELECTROLYTES NOS:Myelofibrosis:0.86), (APREPITANT:Myelofibrosis:0.23), (BALSALAZIDE DISODIUM:Myelofibrosis:0.27), (BETAMETHASONE DIPROPIONATECALCIPOTRIENECALCIPOTRIENE HYDRATE:Myelofibrosis:0.42), (BETAMETHASONECALCIPOTRIENE:Myelofibrosis:0.33), (BISACODYL OR DOCUSATE SODIUM:Myelofibrosis:1.94), (BLINATUMOMAB:Myelofibrosis:0.73), (BUDESONIDEFOR MOTEROL FUMARATE:Myelofibrosis:1.09), (BUPRENORPHINE HYDROCHLORIDE:Myelofibrosis:0.44), (BUPRENORPHINE HYDROCHLORIDENALOXONE HYDROCHLORIDE:Myelofibrosis:0.15), (CALCIPOTRIENE:Myelofibrosis:0.24), (CALCITO NIN SALMON:Myelofibrosis:1.23), (CALCIUM\MAGNESIUM\ZINC:Myelofibrosis:0.32), (CANA GLIFLOZIN:Myelofibrosis:0.17), (CANAGLIFLOZINMETFORMIN HYDROCHLORIDE:Myelofibrosis:0.67), (CANAKINUMAB:Myelofibrosis:1.16), (CARBIMA ZOLE:Myelofibrosis:0.21), (CASANTHRANOLDOCUSATE SODIUM:Myelofibrosis:0.26), (CEFPODOXIME PROXETIL:Myelofibrosis:0.12), (CERTOLIZUMAB PEGOL:Myelofibrosis:1.25), (CHLORAMBUCIL:Myelofibrosis:0.38), (CHLORHEXIDINE:My elofibrosis:0.62), (CHLOROTHIAZIDE:Myelofibrosis:0.02), (CHONDROITIN SULFATE AGLUCOSAMINE:Myelofibrosis:0.29), (CLIKSTAR:Myelofibrosis:0.08), (CLOBAZAM:Myel ofibrosis:0.32), (CLOMIPRAMINE HYDROCHLORIDE:Myelofibrosis:0.07), (DABIGATRAN:Myelofibrosis:0.94), (DACLIZUMA B:Myelofibrosis:1.03), (DEVICE:Myelofibrosis:0.16), (DEXAMETHASONE SODIUM PHOSPHATE:Myelofibrosis:0), (DIPYRONEPROMETHAZINE HYDROCHLORIDE:Myelofibrosis:0.13), (DOLASETRON MESYLATE:Myelofibrosis:0.21), (DORZOLAMIDE HYDROCHLORIDE:Myelofibrosis:0.6), (DOXYLAMINE SUCCINATE:Myelofibrosis:0.01), (ELOTUZUMAB:Myelofibrosis:0.49), (EMPAGLIFLOZIN LINAGLIPTIN:Myelofibrosis:0.44), (ESTROGENS:Myelofibrosis:0.17), (FENOTEROL HYDROBROMIDE:Myelofibrosis:0.48), (FERROUS SULFATEGLYCINE:Myelofibrosis:0.27), (FLUTICASONE FUROATE:Myelofibrosis:0.88), (FLUTICASONE FUROATESALMETEROL XINAFOATE:Myelofibrosis:1.79), (FORMOTEROL FUMARATE\MOMETASONE FUROATE:Myelofibrosis:0.7), (GLYCOPYRROLATE:Myelofibrosis:0.65), (GLYCOPYRROL ATEINDACATEROL MALEATE:Myelofibrosis:0.3), (GLYCOPYRRONIUMINDACATEROL:Myelofibrosis:1.22), (HYDROCODONE BITARTRATEIBUPROFEN:Myelofibrosis:0.62), (HYDROCORTISONE ACETATE:Myelofibrosis:0.55), (ICOSAPENT ETHYL:Myelofibrosis:0.06), (ILOPROST:Myelofibrosis:0.97), (IMIGLUCERASE:Myelofibrosi s:0.15), (IMIPRAMINE:Myelofibrosis:0.07), (INDAPAMIDEPERINDOPRIL:Myelofibrosis:1.42), (INFLIXIMAB-DYYB:Myelofibrosis:0.16), (INSULIN BEEF:Myelofibrosis:0.49), (INSULIN DEGLUDEC:Myelofibrosis:0.05), (INTERFERON GAMMA-1B:Myelofibrosis:0.41), (IRONPOLYSACCHARIDES:Myelofibrosis:0.14), (IVABRADINE HYDROCHLORIDE:Myelofibrosis:0.08), (IXAZOMIB:Myelofibrosis:0.88), (LEDIPASVIRSO FOSBUVIR:Myelofibrosis:1.17), (LEVOMEFOLATE CALCIUM:Myelofibrosis:0.37), (LOMITAPIDE MESYLATE:Myelofibrosis:0.75), (MAGNESIUM SULFATE:Myelofibrosis:0.44), (MEPOLIZUMAB:Myelofibrosis:0.45), (METFORMIN HYDROCHLORIDEROSIGLITAZONE MALEATE:Myelofibrosis:0.18), (METFORMIN HYDROCHLORIDESAXAGLIPTIN HYDROCHLORIDE:Myelofibrosis:0.07), (METFORMIN HYDROCHLORIDEVILDAGLIPTIN:Myelofibrosis:0.41), (METHENAMINE HIPPURATE:Myelofibrosis:1.36), (METHYLPREDNISOLONE ACETATE:Myelofibrosis:1.51), (MORPHINE SULFATE\NALTREXONE HYDROCHLORIDE:Myelofibrosis:0.36), (MOXIFLOXACIN:Myelofibrosis:0.93), (NADROPA RIN CALCIUM:Myelofibrosis:1.31), (NINTEDANIB:Myelofibrosis:1.17), (NIRAPARIB:Myelofibro sis:0.38), (NOREPINEPHRINE BITARTRATE:Myelofibrosis:0.01), (OLOPATADINE:Myelofibrosis:0.03), (ONABOTULINU MTOXINA:Myelofibrosis:0.16), (ORPHENADRINE:Myelofibrosis:0.24), (OXAPROZIN:Myelo fibrosis:0.06), (PALBOCICLIB:Myelofibrosis:0.34), (PALONOSETRON HYDROCHLORIDE:Myelofibrosis:0.11), (PAMIDRONATE DISODIUM:Myelofibrosis:0.1), (PATIROMER:Myelofibrosis:0.73), (PAZOPANIB HYDROCHLORIDE:Myelofibrosis:1.24), (PEGFILGRASTIM:Myelofibrosis:0.3), (PEGINTER FERON BETA-1A:Myelofibrosis:0.51), (PENTAMIDINE:Myelofibrosis:0.07), (PENTAMIDINE ISETHIONATE:Myelofibrosis:0.12), (PENTOSAN POLYSULFATE SODIUM:Myelofibrosis:0.43), (PIRFENIDONE:Myelofibrosis:0.16), (PRAZOSIN HYDROCHLORIDE:Myelofibrosis:0.48), (PREDNISOLONE SODIUM PHOSPHATE:Myelofibrosis:0.34), (PROTRIPTYLINE HYDROCHLORIDE:Myelofibrosis:0.07), (RADIATION THERAPY:Myelofibrosis:0.08), (REGORAFENIB:Myelofibrosis:0.13), (SACUBITRILVALSA RTAN:Myelofibrosis:0.34), (SECUKINUMAB:Myelofibrosis:0.67), (SELEGILINE:Myelofibrosi s:0.18), (SELEXIPAG:Myelofibrosis:0.37), (SODIUM OXYBATE:Myelofibrosis:1.52), (SOFOSBUVIRVELPATASVIR:Myelofibrosis:0.23), (STAR CH, WHEAT:Myelofibrosis:0.11), (SUNITINIB MALATE:Myelofibrosis:0.66), (TEDUGLUTIDE\WATER:Myelofibrosis:0.56), (TERBUTALIN E:Myelofibrosis:0.14), (TESTOSTERONE UNDECANOATE:Myelofibrosis:0.24), (THEOPHYLLINE:Myelofibrosis:0.06), (TIGECYCLIN E:Myelofibrosis:0.14), (TRIMIPRAMINE MALEATE:Myelofibrosis:0.16), (BUDESONIDE:Myeloid leukaemia:0.15), (BUMETANIDE:Myeloid leukaemia:0.13), (CITALOPRAM HYDROBROMIDE:Myeloid leukaemia:0.01), (CLONAZEPAM:Myeloid leukaemia:0.16), (DEXLANSOPRAZOLE:Myeloid leukaemia:0.07), (DILTIAZEM HYDROCHLORIDE:Myeloid leukaemia:0.55), (ENALAPRIL:Myeloid leukaemia:0.58), (LANSOPRAZOLE:Myeloid leukaemia:0.12), (SACUBITRILVALSARTAN:Myeloid leukaemia:0.1), (ACLIDINIUM BROMIDE:Myeloproliferative neoplasm:0.06), (AMPHETAMINE ASPARTATEAMPHETAMINE SULFATEDEXTROAMPHETAMINE SACCHARATEDEXTROAMPHETAMINE SULFATE:Myeloproliferative neoplasm:0.09), (ANASTROZOLE:Myeloproliferative neoplasm:0.01), (BISACODYL OR DOCUSATE SODIUM:Myeloproliferative neoplasm:0.1), (BUDESONIDE:Myeloproliferative neoplasm:1.06), (CANDESARTAN:Myeloproliferative neoplasm:0.41), (CANDESARTAN CILEXETILHYDROCHLOROTHIAZIDE:Myeloproliferative neoplasm:0.2), (CARFILZOMIB:Myeloproliferative neoplasm:0.2), (CEFTRIAXONE SODIUM:Myeloproliferative neoplasm:0.08), (CERTOLIZUMAB PEGOL:Myeloproliferative neoplasm:0.22), (CLONIDINE:Myeloproliferative neoplasm:0.24), (CYCLOBENZAPRINE HYDROCHLORIDE:Myeloproliferative neoplasm:0.36), (DICLOFENAC:Myeloproliferative neoplasm:0.57), (DOFETILIDE:Myeloproliferative neoplasm:0.07), (EPINEPHRINE:Myeloproliferative neoplasm:0.73), (ESOMEPRAZOLE:Myeloproliferative neoplasm:0.16), (ESZOPICLONE:Myeloproliferative neoplasm:0.21), (EVEROLIMUS:Myeloproliferative neoplasm:0.24), (EVOLOCUMAB:Myeloproliferative neoplasm:0.55), (EZETIMIBE:Myeloproliferative neoplasm:0.46), (FERROUS SULFATEFERROUS SULFATE, DRIED:Myeloproliferative neoplasm:1.15), (FESOTERODINE FUMARATE:Myeloproliferative neoplasm:0.06), (GLYBURIDE:Myeloproliferative neoplasm:0.05), (HYOSCYAMINE:Myeloproliferative neoplasm:0.02), (IBRUTINIB:Myeloproliferative neoplasm:1.07), (ILOPROST:Myeloproliferative neoplasm:0.33), (INSULIN HUMAN:Myeloproliferative neoplasm:0.04), (LINAGLIPTIN:Myeloproliferative neoplasm:0.03), (MELATONIN:Myeloproliferative neoplasm:0.31), (METAXALONE:Myeloproliferative neoplasm:0.28), (METHYLPREDNISOLONE SODIUM SUCCINATE:Myeloproliferative neoplasm:0.52), (NIACIN:Myeloproliferative neoplasm:0.04), (OMEGA-3-ACID ETHYL ESTERS:Myeloproliferative neoplasm:0.36), (OXYGEN:Myeloproliferative neoplasm:0.92), (PANTOPRAZOLE MAGNESIUM:Myeloproliferative neoplasm:0.31), (PRAMIPEXOLE DIHYDROCHLORIDE:Myeloproliferative neoplasm:0.18), (SACUBITRILVALSARTAN:Myeloproliferative neoplasm:0.56), (SULFAMETHOXAZOLE:Myeloproliferative neoplasm:0.18), (TERIFLUNOMIDE:Myeloproliferative neoplasm:0.23), (TETRACYCLINE:Myeloproliferative neoplasm:0.14), (TIMOLOL:Myeloproliferative neoplasm:0.41), (TIZANIDINE HYDROCHLORIDE:Myeloproliferative neoplasm:0.02), (TRIAMCINOLONE ACETONIDE:Myeloproliferative neoplasm:0.66), (.ALPHA.1-PROTEINASE INHIBITOR HUMAN:Myocardial infarction:0.59), (ABOBOTULINUMTOXINA:Myocardial infarction:0.13), (ACETAMINOPHEN\CAFFEINE:Myocardial infarction:0.46), (ADO-TRASTUZUMAB EMTANSINE:Myocardial infarction:1.1), (AGALSIDASE BETA:Myocardial infarction:1.23), (ALBIGLUTIDE:Myocardial infarction:0.22), (ALBUMIN HUMAN:Myocardial infarction:0.24), (ALBUTEROLIPRATROPIUM:Myocardial infarction:0.31), (ALISKIREN HEMIFUMARATE:Myocardial infarction:1.14), (AMANTADINE:Myocardial infarction:0.61), (AMANTADINE HYDROCHLORIDE:Myocardial infarction:0.68), (AMBROXOLCLENBUTEROL:Myocardial infarction:0.02), (AMINO ACIDSDEXTROSEELECTROLYTES NOS:Myocardial infarction:0.36), (AMISULPRIDE:Myocardial infarction:0.79), (AMLODIPINE BESYLATEHYDROCHLOROTHIAZIDEOLMESARTAN MEDOXOMIL:Myocardial infarction:0.52), (ANAGRELIDE HYDROCHLORIDE:Myocardial infarction:0.52), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Myocardial infarction:0.09), (ASUNAPREVIR:Myocardial infarction:0.07), (AVANAFIL:Myocardial infarction:0.01), (BETAMETHASONE DIPROPIONATECALCIPOTRIENECALCIPOTRIENE HYDRATE:Myocardial infarction:0.39), (BETAMETHASONE DIPROPIONATECLOTRIMAZOLE:Myocardial infarction:0.21), (BETHANECHOL CHLORIDE:Myocardial infarction:0.51), (BIFIDOBACTERIUM SPP.:Myocardial infarction:0.33), (BUPRENORPHINE HYDROCHLORIDENALOXONE HYDROCHLORIDE:Myocardial infarction:0.67), (BUTYLSCOPOLAMINE BROMIDE:Myocardial infarction:0.22), (CABAZITAXEL:Myocardial infarction:0.28), (CALCITONIN:Myocardial infarction:0.38), (CALCITONIN SALMON:Myocardial infarction:0.9), (CALCIUM\MAGNESIUM:Myocardial infarction:0.22), (CANAGLIFLOZINMETFORMIN HYDROCHLORIDE:Myocardial infarction:1.44), (CARBIDOPA:Myocardial infarction:0.36), (CARBOMER:Myocardial infarction:0.14), (CEFEPIME HYDROCHLORIDE:Myocardial infarction:0.05), (CERITINIB:Myocardial infarction:0.21), (CETIRIZINE HYDROCHLORIDEPSEUDOEPHEDRINE HYDROCHLORIDE:Myocardial infarction:0.4), (CHLORDIAZEPOXIDE HYDROCHLORIDECLIDINIUM BROMIDE:Myocardial infarction:0.46), (CHLOROTHIAZIDE:Myocardial infarction:0.2), (CICLESONIDE:Myocardial infarction:0.87), (CIFENLINE:Myocardial infarction:0.54), (CLIKSTAR:Myocardial infarction:0.26), (CLINDAMYCIN HYDROCHLORIDE:Myocardial infarction:0.06), (CLOMIPRAMINE HYDROCHLORIDE:Myocardial infarction:0.17), (COD LIVER OIL:Myocardial infarction:0.29), (CODEINE PHOSPHATEGUAIFENESIN:Myocardial infarction:0.23), (CORTICOTROPIN:Myocardial infarction:0.65), (CRANBERRY:Myocardial infarction:0.09), (CRIZOTINIB:Myocardial infarction:1.08), (CYPROHEPTADINE:Myocardial infarction:0.11), (DABIGATRAN ETEXILATE:Myocardial infarction:0.44), (DACLATASVIR:Myocardial infarction:0.1), (DALTEPARIN:Myocardial infarction:1.26), (DANTROLENE SODIUM:Myocardial infarction:0.12), (DEFLAZACORT:Myocardial infarction:1.27), (DESVENLAFAXINE SUCCINATE:Myocardial infarction:0.88), (DIGITALIS:Myocardial infarction:0.26), (DIPYRONE:Myocardial infarction:0.33), (DISOPYRAMIDE:Myocardial infarction:0.33), (DOLASETRON MESYLATE:Myocardial infarction:0.13), (DOXYCYCLINE HYCLATE:Myocardial infarction:0.19), (DOXYLAMINE SUCCINATE:Myocardial infarction:0.06), (DRONABINOL:Myocardial infarction:1.28), (ECONAZOLE:Myocardial infarction:1.14), (EDARAVONE:Myocardial infarction:0.62), (ELETRIPTAN HYDROBROMIDE:Myocardial infarction:1.42), (ELOTUZUMAB:Myocardial infarction:0.08), (EPALRESTAT:Myocardial infarction:0.17), (EPERISONE HYDROCHLORIDE:Myocardial infarction:0), (EPINASTINE HYDROCHLORIDE:Myocardial infarction:0.67), (EPIRUBICIN:Myocardial infarction:1.03), (ERLOTINIB:Myocardial infarction:0.03), (ESTROGENS, CONJUGATEDMEDROXYPROGESTERONE ACETATE:Myocardial infarction:0.67), (ETRETINATE:Myocardial infarction:0.23), (FERRIC CITRATE ANHYDROUS:Myocardial infarction:0.26), (FERRIC HYDROXIDE:Myocardial infarction:0.99), (FERROUS SULFATEGLYCINE:Myocardial infarction:0.14), (FLUOCINONIDE:Myocardial infarction:0.67), (FLURBIPROFEN:Myocardial infarction:0.07), (FORMOTEROL:Myocardial infarction:0.8), (FORMOTEROL FUMARATE\MOMETASONE FUROATE:Myocardial infarction:0.4), (FULVESTRANT:Myocardial infarction:0.35), (FURSULTIAMINE:Myocardial infarction:0.32), (GENTAMICIN:Myocardial infarction:0.19), (GLIMEPIRIDEROSIGLITAZONE MALEATE:Myocardial infarction:0.96), (GLYCOPYRROLATEINDACATEROL MALEATE:Myocardial infarction:0.36), (GUANFACINE:Myocardial infarction:0.19), (HERBALSTURMERIC:Myocardial infarction:0.77), (HYDROCHLOROTHIAZIDETELMISARTAN:Myocardial infarction:1.64), (HYDROCODONE BITARTRATEIBUPROFEN:Myocardial infarction:0.22), (HYDROXYCHLOROQUINE DIPHOSPHATE:Myocardial infarction:0.54), (ILOPROST:Myocardial infarction:1.56), (INFLIXIMAB-DYYB:Myocardial infarction:0.05), (INSULIN ASPARTINSULIN DEGLUDEC:Myocardial infarction:0.42), (INSULIN PORKINSULIN PURIFIED PORK:Myocardial infarction:0.12), (INTERFERON:Myocardial infarction:0.07), (INTERFERON ALFA-2A:Myocardial infarction:0.69), (INTERFERON ALFA-2B:Myocardial infarction:0.2), (IODINE:Myocardial infarction:0.35), (IPRAGLIFLOZIN L-PROLINE:Myocardial infarction:0.29), (IRINOTECAN HYDROCHLORIDE:Myocardial infarction:1.29), (IRON SUCROSE:Myocardial infarction:0.63), (LABETALOL HYDROCHLORIDE:Myocardial infarction:0.99), (LANREOTIDE:Myocardial infarction:0.02), (LANREOTIDE ACETATE:Myocardial infarction:0.56), (LAPATINIB DITOSYLATE:Myocardial infarction:0.4), (LENVATINIB:Myocardial infarction:0.17), (LEVOSIMENDAN:Myocardial infarction:0.13), (LIDOCAINEPRILOCAINE:Myocardial infarction:0.85), (LIOTHYRONINE:Myocardial infarction:0.18), (LITHIUM:Myocardial infarction:0.55), (LURASIDONE HYDROCHLORIDE:Myocardial infarction:0.01), (MAGNESIUM ASPARTATE HYDROCHLORIDE:Myocardial infarction:0.14), (MAXACALCITOL:Myocardial infarction:0.08), (MEPOLIZUMAB:Myocardial infarction:0.35), (MESNA:Myocardial infarction:0.72), (METFORMINSITAGLIPTIN:Myocardial infarction:0.06), (METHENOLONE ENANTHATE:Myocardial infarction:0.13), (METILDIGOXIN:Myocardial infarction:0.49), (METRELEPTIN:Myocardial infarction:0.03), (MINOCYCLINEMINOCYCLINE HYDROCHLORIDE:Myocardial infarction:1.46), (MITIGLINIDE CALCIUM HYDRATEVOGLIBOSE:Myocardial infarction:0.19), (MORPHINE SULFATE\NALTREXONE HYDROCHLORIDE:Myocardial infarction:0.2), (MOXIFLOXACIN:Myocardial infarction:0.7), (NALOXONE HYDROCHLORIDETILIDINE HYDROCHLORIDE:Myocardial infarction:1.29), (NICARDIPINE:Myocardial infarction:1.1), (NIRAPARIB:Myocardial infarction:0.68), (OLAPARIB:Myocardial infarction:0.13), (OLODATEROL HYDROCHLORIDETIOTROPIUM BROMIDE MONOHYDRATE:Myocardial infarction:1.39), (OMEGA-3-CARBOXYLIC ACIDS:Myocardial infarction:0.34), (ORPHENADRINE:Myocardial infarction:0.19), (OSELTAMIVIR:Myocardial infarction:0.04), (OSELTAMIVIR PHOSPHATE:Myocardial infarction:0.19), (OXYBUTYNINOXYBUTYNIN CHLORIDE:Myocardial infarction:0.32), (PAMIDRONATE DISODIUM:Myocardial infarction:0.26), (PANITUMUMAB:Myocardial infarction:0.63), (PATIROMER:Myocardial infarction:0.83), (PEGZEREPOETIN ALFA:Myocardial infarction:0.55), (PEMETREXED DISODIUM:Myocardial infarction:1.12), (PENICILLIN G:Myocardial infarction:0.13), (PENICILLIN V:Myocardial infarction:0.07), (PETROLATUM:Myocardial infarction:0.53), (PHYTONADIONE:Myocardial infarction:0.8), (POMALIDOMIDE:Myocardial infarction:0.03), (PRAZOSIN:Myocardial infarction:0.7), (PRAZOSIN HYDROCHLORIDE:Myocardial infarction:0.66), (PRISTINAMYCIN:Myocardial infarction:0.41), (PYRAZINAMIDE:Myocardial infarction:0.75), (RADIATION THERAPY:Myocardial infarction:0.4), (RETEPLASE:Myocardial infarction:0.23), (RETINOL:Myocardial infarction:0.33), (RILMENIDINE PHOSPHATE:Myocardial infarction:0.15), (RIZATRIPTAN:Myocardial infarction:0.05), (RIZATRIPTAN BENZOATE:Myocardial infarction:0.88), (ROSIGLITAZONE:Myocardial infarction:0.21), (CEREVISIAE:Myocardial infarction:0.54), (SELEGILINE HYDROCHLORIDE:Myocardial infarction:0.06), (SELEXIPAG:Myocardial infarction:0.02), (SILVER SULFADIAZINE:Myocardial infarction:0.36), (SODIUM FLUORIDE:Myocardial infarction:0.26), (SODIUM PHOSPHATE, DIBASIC\SODIUM PHOSPHATE, MONOBASIC:Myocardial infarction:0.53), (SULINDAC:Myocardial infarction:0.06), (SUMATRIPTANSUMATRIPTAN SUCCINATE:Myocardial infarction:0.98), (TAZOBACTAM:Myocardial infarction:0.14), (TERBUTALINE:Myocardial infarction:0.85), (TERBUTALINE SULFATE:Myocardial infarction:1.85), (TESTOSTERONE ENANTHATE:Myocardial infarction:0.22), (TESTOSTERONE UNDECANOATE:Myocardial infarction:0.69), (TETRACYCLINE:Myocardial infarction:1.06), (THALIDOMIDE:Myocardial infarction:1.68), (THIOCTIC ACID:Myocardial infarction:0.57), (TIMOLOL MALEATETRAVOPROST:Myocardial infarction:0.06), (TIPIRACIL HYDROCHLORIDETRIFLURIDINE:Myocardial infarction:0.06), (TOBRAMYCIN:Myocardial infarction:0.25), (TOLTERODINE:Myocardial infarction:0.4), (TRIMIPRAMINE MALEATE:Myocardial infarction:0.3), (TRIPTORELIN PAMOATE:Myocardial infarction:0.18), (TROSPIUM CHLORIDE:Myocardial infarction:1.18), (UMECLIDINIUM BROMIDE:Myocardial infarction:0.53), (VALGANCICLOVIR HYDROCHLORIDE:Myocardial infarction:0.58), (VANCOMYCIN HYDROCHLORIDE:Myocardial infarction:0.51), (VINORELBINE TARTRATE:Myocardial infarction:0.23), (ACETAMINOPHENBUTALBITALCAFFEINE:Myocardial ischaemia:0.27), (ACETAMINOPHENHYDROCODONE:Myocardial ischaemia:0.09), (ACETAMINOPHENOXYCODONEOXYCODONE HYDROCHLORIDE:Myocardial ischaemia:0.05), (ACTIVATED CHARCOAL:Myocardial ischaemia:0.38), (ALCOHOL:Myocardial ischaemia:0.14), (AMIKACIN SULFATE:Myocardial ischaemia:0.03), (AMISULPRIDE:Myocardial ischaemia:0.49), (AMITRIPTYLINE HYDROCHLORIDE:Myocardial ischaemia:0.29), (AMLODIPINEVALSARTAN:Myocardial ischaemia:0.19), (AMPHETAMINE ASPARTATEAMPHETAMINE SULFATEDEXTROAMPHETAMINE SACCHARATEDEXTROAMPHETAMINE SULFATE:Myocardial ischaemia:0.14), (ANAGRELIDE HYDROCHLORIDE:Myocardial ischaemia:0.07), (ATROPINE SULFATEDIPHENOXYLATE HYDROCHLORIDE:Myocardial ischaemia:0.3), (AZELNIDIPINE:Myocardial ischaemia:0.2), (BENAZEPRIL HYDROCHLORIDE:Myocardial ischaemia:0.98), (BENAZEPRILBENAZEPRIL HYDROCHLORIDE:Myocardial ischaemia:0.45), (BENSERAZIDELEVODOPA:Myocardial ischaemia:0.03), (BEPRIDIL:Myocardial ischaemia:0.22), (BUDESONIDEFORMOTEROL FUMARATE:Myocardial ischaemia:0.77), (BUPRENORPHINE HYDROCHLORIDE:Myocardial ischaemia:0.55), (BUPROPION:Myocardial ischaemia:0.6), (CABAZITAXEL:Myocardial ischaemia:0.04), (CALCIUM CITRATE:Myocardial ischaemia:0.44), (CANAGLIFLOZINMETFORMIN HYDROCHLORIDE:Myocardial ischaemia:0.48), (CEFAZOLIN:Myocardial ischaemia:0.04), (CEFAZOLIN SODIUM:Myocardial ischaemia:0.07), (CERITINIB:Myocardial ischaemia:0.21), (CETUXIMAB:Myocardial ischaemia:1.36), (CICLESONIDE:Myocardial ischaemia:0.13), (CIMETIDINE:Myocardial ischaemia:0.36), (CORTISONEHYDROCORTISONE:Myocardial ischaemia:0.94), (CRIZOTINIB:Myocardial ischaemia:0.99), (CYAMEMAZINE:Myocardial ischaemia:0.14), (DANAZOL:Myocardial ischaemia:0.27), (DAPAGLIFLOZIN PROPANEDIOL:Myocardial ischaemia:0.39), (DASATINIB:Myocardial ischaemia:2.05), (DEXAMETHASONE ACETATE:Myocardial ischaemia:0.41), (DICYCLOMINE HYDROCHLORIDE:Myocardial ischaemia:0.35), (DIMENHYDRINATE:Myocardial ischaemia:0.18), (DIPHENHYDRAMINE:Myocardial ischaemia:0.93), (DIPHENHYDRAMINE HYDROCHLORIDE:Myocardial ischaemia:0.48), (DOCUSATE CALCIUM:Myocardial ischaemia:0.06), (DRONABINOL:Myocardial ischaemia:0.22), (DUTASTERIDETAMSULOSIN HYDROCHLORIDE:Myocardial ischaemia:0.45), (ECONAZOLE:Myocardial ischaemia:0.98), (ELETRIPTAN HYDROBROMIDE:Myocardial ischaemia:0.02), (ELOTUZUMAB:Myocardial ischaemia:0.14), (EMPAGLIFLOZINLINAGLIPTIN:Myocardial ischaemia:0.1), (EMPAGLIFLOZINMETFORMIN HYDROCHLORIDE:Myocardial ischaemia:0.05), (EPIRUBICIN:Myocardial ischaemia:0.9), (ESZOPICLONE:Myocardial ischaemia:0.79), (FERRIC HYDROXIDE:Myocardial ischaemia:0.29), (FLUDROCORTISONE ACETATE:Myocardial ischaemia:0.25), (FONDAPARINUX:Myocardial ischaemia:0.06), (FORMOTEROL FUMARATE DIHYDRATE\MOMETASONE FUROATE:Myocardial ischaemia:0.23), (FOSFOMYCIN:Myocardial ischaemia:0.11), (GLIMEPIRIDEROSIGLITAZONE MALEATE:Myocardial ischaemia:0.18), (GLYCOPYRROLATEINDACATEROL MALEATE:Myocardial ischaemia:0.01), (GUAIFENESIN:Myocardial ischaemia:0.41), (HYDROCHLOROTHIAZIDELISINOPRIL:Myocardial ischaemia:1.42), (HYDROCODONE:Myocardial ischaemia:0.85), (HYDROMORPHONE:Myocardial ischaemia:0.79), (HYDROXYZINE PAMOATE:Myocardial ischaemia:0.5), (IDARUCIZUMAB:Myocardial ischaemia:0.58), (ILOPROST:Myocardial ischaemia:1.56), (IMIPRAMINE HYDROCHLORIDE:Myocardial ischaemia:0.08), (INTERFERON ALFA-2A:Myocardial ischaemia:0.12), (IOPAMIDOL:Myocardial ischaemia:0.04), (IRINOTECAN HYDROCHLORIDE:Myocardial ischaemia:1.14), (IXAZOMIB:Myocardial ischaemia:0.27), (LABETALOL HYDROCHLORIDE:Myocardial ischaemia:0.38), (LABETALOLLABETALOL HYDROCHLORIDE:Myocardial ischaemia:1.11), (LAFUTIDINE:Myocardial ischaemia:0.05), (LAPATINIB DITOSYLATE:Myocardial ischaemia:0.15), (LETROZOLE:Myocardial ischaemia:0.65), (LEVALBUTEROL HYDROCHLORIDE:Myocardial ischaemia:0.86), (MEGESTROL ACETATE:Myocardial ischaemia:0.33), (MELPERONE:Myocardial ischaemia:0.17), (METFORMIN HYDROCHLORIDEROSIGLITAZONE MALEATE:Myocardial ischaemia:0.32), (METFORMINSITAGLIPTIN:Myocardial ischaemia:0.02), (METHOCARBAMOL:Myocardial ischaemia:0.13), (METHYLPREDNISOLONE ACETATE:Myocardial ischaemia:0.15), (MIDODRINE:Myocardial ischaemia:0.7), (MINOCYCLINEMINOCYCLINE HYDROCHLORIDE:Myocardial ischaemia:0.57), (NALOXONE HYDROCHLORIDETILIDINE HYDROCHLORIDE:Myocardial ischaemia:0.77), (NOREPINEPHRINE:Myocardial ischaemia:0.06), (NOREPINEPHRINE BITARTRATE:Myocardial ischaemia:0.05), (OXYBUTYNIN CHLORIDE:Myocardial ischaemia:0.32), (PATIROMER:Myocardial ischaemia:0.18), (PEGINTERFERON BETA-1A:Myocardial ischaemia:0.04), (PEMETREXED DISODIUM:Myocardial ischaemia:0.92), (PENICILLIN:Myocardial ischaemia:0.31), (PHENYTOIN:Myocardial ischaemia:0.77), (PHENYTOIN SODIUM:Myocardial ischaemia:0.17), (PIOGLITAZONE:Myocardial ischaemia:0.13), (PIRITRAMIDE:Myocardial ischaemia:0.04), (PREDNISOLONEPREDNISOLONE ACETATEPREDNISOLONE SODIUM SUCCINATE:Myocardial ischaemia:1.17), (PRISTINAMYCIN:Myocardial ischaemia:0.53), (PYRIDOSTIGMINE BROMIDE:Myocardial ischaemia:0.01), (RALTEGRAVIR POTASSIUM:Myocardial ischaemia:0.2), (RIFAMPIN:Myocardial ischaemia:0.79), (RIFAXIMIN:Myocardial ischaemia:0.11), (RIOCIGUAT:Myocardial ischaemia:1.07), (ROFLUMILAST:Myocardial ischaemia:0.92), (ROPINIROLE HYDROCHLORIDE:Myocardial ischaemia:0.36), (ROSIGLITAZONE MALEATE:Myocardial ischaemia:0.56), (SOTALOL HYDROCHLORIDE:Myocardial ischaemia:0.36), (SULFAMETHOXAZOLE:Myocardial ischaemia:0.37), (TAFAMIDIS MEGLUMINE:Myocardial ischaemia:0.19), (TAZOBACTAM:Myocardial ischaemia:0.19), (TERBINAFINE HYDROCHLORIDE:Myocardial ischaemia:0.17), (TESTOSTERONE CYPIONATE:Myocardial ischaemia:0.12), (TETRACYCLINE:Myocardial ischaemia:0.49), (THALIDOMIDE:Myocardial ischaemia:1.14), (TINZAPARIN:Myocardial ischaemia:0.9), (TIZANIDINE HYDROCHLORIDE:Myocardial ischaemia:0.55), (TRIAMTERENE:Myocardial ischaemia:0.09), (UMECLIDINIUM BROMIDEVILANTEROL TRIFENATATE:Myocardial ischaemia:0.43), (VANCOMYCIN HYDROCHLORIDE:Myocardial ischaemia:0.52), (VINCRISTINE SULFATE:Myocardial ischaemia:0.37), (PYRAZINAMIDE:Myocarditis:0.59), (FISH OIL:Myoclonus:0.15), (OXCARBAZEPINE:Myoclonus:0.9), (ACETAMINOPHENCODEINE PHOSPHATE:Myositis:0.26), (ANASTROZOLE:Myositis:0.07), (BUDESONIDE:Myositis:0.6), (BUMETANIDE:Myositis:0.13), (CERTOLIZUMAB PEGOL:Myositis:0.97), (COLCHICINE:Myositis:0.07), (DALFAMPRIDINE:Myositis:0.11), (DI LTIAZEM:Myositis:0.34), (DILTIAZEM HYDROCHLORIDE:Myositis:0.05), (DIVALPROEX SODIUM:Myositis:0.55), (ENOXAPARIN:Myositis:0.39), (ESZOPICLONE:Myositis:0.22), (EV OLOCUMAB:Myositis:0.34), (FERROUS SULFATEFERROUS SULFATE, DRIED:Myositis:0.4), (HYDROXYZINE PAMOATE:Myositis:0.13), (IBRUTINIB:Myositis:0.61), (INDOMETHACIN:Myositis:0.24), (IN SULIN ASPART:Myositis:0.1), (IRON:Myositis:0.55), (LISDEXAMFETAMINE DIMESYLATE:Myositis:0.35), (MAGNESIUM:Myositis:0.15), (METRONIDAZOLE:Myositis: 0.67), (OLANZAPINE:Myositis:0.6), (PANTOPRAZOLE:Myositis:0.6), (PAROXETINEPARO XETINE HYDROCHLORIDE:Myositis:0.12), (POLYETHYLENE GLYCOLS:Myositis:0.12), (PRIMIDONE:Myositis:0.01), (RABEPRAZOLE SODIUM:Myositis:0.08), (RIZATRIPTAN BENZOATE:Myositis:0.04), (SACUBITRILVALSARTAN:Myositis:0.21), (SUMATRIPTANS UMATRIPTAN SUCCINATE:Myositis:0.31), (TERIFLUNOMIDE:Myositis:0.12), (VITAMIN B:Myositis:0.03), (ALBUTEROL:Nail disorder:0.6), (ALLOPURINOL:Nail disorder:0.41), (AMIODARONE:Nail disorder:0.01), (AMOXICILLIN:Nail disorder:0.24), (BICALUTAMIDE:Nail disorder:0.48), (BISACODYL OR DOCUSATE SODIUM:Nail disorder:0.06), (BUDESONIDE:Nail disorder:0.85), (CLINDAMYCINCLINDAMYCIN PHOSPHATE:Nail disorder:0.17), (CYCLOBENZAPRINE HYDROCHLORIDE:Nail disorder:0.4), (DALFAMPRIDINE:Nail disorder:0.27), (DEVICE:Nail disorder:0.03), (DICLOFENAC:Nail disorder:0.3), (DIGOXIN:Nail disorder:0.55), (DILTIAZEM HYDROCHLORIDE:Nail disorder:1.24), (ELETRIPTAN HYDROBROMIDE:Nail disorder:0.23), (ESZOPICLONE:Nail disorder:0.34), (EVOLOCUMAB:Nail disorder:0.88), (FERROUS SULFATEFERROUS SULFATE, DRIED:Nail disorder:0.54), (HYDROXYCHLOROQUINE DIPHOSPHATE:Nail disorder:0.03), (HYDROXYZINE PAMOATE:Nail disorder:0.07), (IBRUTINIB:Nail disorder:0.44), (INDOMETHACIN:Nail disorder:0.1), (LISDEXAMFETAMINE DIMESYLATE:Nail disorder:0.43), (LITHIUM CARBONATE:Nail disorder:0.08), (METHYLPREDNISOLONE SODIUM SUCCINATE:Nail disorder:0.11), (MODAFINIL:Nail disorder:0.26), (OMEGA-3-ACID ETHYL ESTERS:Nail disorder:0.05), (PAROXETINE:Nail disorder:0.27), (PENICILLIN:Nail disorder:0.06), (POLYETHYLENE GLYCOLS:Nail disorder:0.38), (PROMETHAZINEPROMETHAZINE HYDROCHLORIDE:Nail disorder:0.33), (RALOXIFENE HYDROCHLORIDE:Nail disorder:0.21), (RIZATRIPTAN BENZOATE:Nail disorder:0.01), (SACUBITRILVALSARTAN:Nail disorder:0.23), (SECUKINUMAB:Nail disorder:0.09), (.ALPHA.-TOCOPHEROL ACETATE, D-ASCORBIC ACIDCALCIUM PANTOTHENATEFOLIC ACIDMANGANESENIACINAMIDEPYRIDOXINE\RIBOFLAVINTHIAMINEVITAMIN A\ZINC OXIDE:Narcolepsy:0.67), (.ALPHA.-TOCOPHEROLVITAMINS:Narcolepsy:0.48), (.BETA.-CAROTENE:Narcolepsy:0.24), (2-HYDROXYETHYL METHACRYLATE:Narcolepsy:0), (ABALOPARATIDE:Narcolepsy:0.3), (ABOBOTULINUM TOXINA:Narcolepsy:0.57), (ACECLOFENAC:Narcolepsy:0.16), (ACETAMINOPHENASPIRI N\CAFFEINECODEINE PHOSPHATE:Narcolepsy:0.55), (ACETAMINOPHEN\ASPIRINDIPHENHYDRAMINE CITRATE:Narcolepsy:0.14), (ACETAMINOPHENBUCLIZINE HYDROCHLORIDECODEINE:Narcolepsy:0.08), (ACETAMINOPHENBUTABARBITALC AFFEINE:Narcolepsy:0.04), (ACETAMINOPHEN\CAFFEINECARISOPRODOLDICLOFEN AC:Narcolepsy:0.5), (ACETAMINOPHEN\CAFFEINECARISOPRODOLDICLOFENAC SODIUM:Narcolepsy:0.09), (ACETAMINOPHENCHLORPHENIRAMINE MALEATEDEXTROMETHORPHAN HYDROBROMIDE:Narcolepsy:0.08), (ACETAMINOPHENCHLORPHENIRAMINE MALEATEDEXTROMETHORPHAN HYDROBROMIDEPHENYLEPHRINE HYDROCHLORIDE OR ACETAMINOPHENDEXTROMETHORPHAN HYDROBROMIDEDOXYLAMINE SUCCINATEPHENYLEPHRINE HYDROCHLORIDE OR HOMEOPATHICS:Narcolepsy:0.47), (ACETAMINOPHENCHLORPHENIRAMINE MALEATEPHENYLEPHRINE HYDROCHLORIDE OR CETIRIZINE HYDROCHLORIDE OR CHLORPHENIRAMINE MALEATE OR CLEMASTINE FUMARATE OR DIPHENHYDRAMINE HYDROCHLORIDE OR FEXOFENADINE HYDROCHLORIDE OR LORATADINE:Narcolepsy:0.42), (ACETAMINOPHENCHLORPHENIRAMINE MALEATEPSEUDOEPHEDRINE HYDROCHLORIDE:Narcolepsy:0.78), (ACETAMINOPHENMETHOCARBAMOL:Narcoleps y:0.7), (ACETAMINOPHENPHENYLEPHRINE HYDROCHLORIDE:Narcolepsy:0.04), (AJMALINE:Narcolepsy:0.51), (ALBUTEROLIPRATR OPIUM BROMIDE:Narcolepsy:0.2), (ALCLOMETASONE:Narcolepsy:0.2), (ALENDRONIC ACID:Narcolepsy:1.08), (ALFACALCIDOL:Narcolepsy:0.41), (ALGINIC ACIDALUMINUM HYDROXIDEMAGNESIUM CARBONATE:Narcolepsy:0.19), (ALLANTOIN:Narcolepsy:0.03), (ALUMINUM HYDROXIDEMAGNESIUM HYDROXIDESIMETHICONE:Narcolepsy:0.72), (AMILORIDE HYDROCHLORIDEFUROSEMIDE:Narcolepsy:0.52), (AMINO ACIDSDEXTROSEELECTROLYTES NOS:Narcolepsy:0.95), (AMINOBENZOIC ACIDCALCIUM PANTOTHENATECYSTINEKERATINTHIAMINE MONONITRATE:Narcolepsy:0.28), (AMISULPRIDE:Narcolepsy:0.56), (AMITRIPTYLINEPE RPHENAZINE:Narcolepsy:0.72), (ANTHRALIN:Narcolepsy:0.15), (ARABICA COFFEE BEANCOFFEE BEAN:Narcolepsy:0.09), (ARCTOSTAPHYLOS UVA-URSI LEAFDIOICA:Narcolepsy:0.22), (ARIPIPRAZOLE LAUROXIL:Narcolepsy:1.43), (ASCORBIC ACID/CHROMIUM/SELENIUM/VANADYL SULFATE:Narcolepsy:1.68), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE MONONITRATE:Narcolepsy:0.3), (ASCORBIC ACIDCHONDROITIN SULFATE (BOVINE)GLUCOSAMINE SULFATEMANGANESE:Narcolepsy:0.09), (ASPIRINDIHYDROXYALUMINUM AMINOACETATE ANHYDROUSMAGNESIUM CARBONATE:Narcolepsy:0.81), (ATENOLOLHYDROCHLOROTHIAZIDE:Narcolepsy:0.87), (BALSALAZIDE:Narcolepsy:0.47), (BAMIFYLLINE HYDROCHLORIDE:Narcolepsy:0.63), (BECLOMETHASONEFORMOTEROL:Narcolepsy:0.54), (BENDROFLUMETHIAZIDE:Narcolepsy:0.64), (BEPOTASTINEBEPOTASTINE BESILATE:Narcolepsy:0.27), (BETA GLUCAN:Narcolepsy:0.25), (BETAMETHASONE DIPROPIONATECALCIPOTRIENE:Narcolepsy:0.19), (BETAMETHASONECALCIPOTRIE NE:Narcolepsy:1.01), (BIPERIDEN HYDROCHLORIDE:Narcolepsy:0.08), (BISMUTH SUBSALICYLATEGLYCERINPROPYLENE GLYCOL:Narcolepsy:0.18), (BORTEZOMIB:Narcolepsy:0.14), (BRINZOLAMIDETIMOLOL MALEATE:Narcolepsy:0.01), (BRIVARACETAM:Narcolepsy:0.33), (BUDESONIDEFORMO TEROL:Narcolepsy:0.19), (BUPRENORPHINE HYDROCHLORIDENALOXONE HYDROCHLORIDE DIHYDRATE:Narcolepsy:0.57), (BUPRENORPHINENALOXONE:Narcolepsy:1.78), (BUTAB ARBITAL:Narcolepsy:1.63), (BUTYLSCOPOLAMINE BROMIDE:Narcolepsy:0.84), (CABOZANTINIB S-MALATE:Narcolepsy:0.29), (CAFFEINEDIPYRONEORPHENADRINE:Narcolepsy:0.61), (C ALCITONIN:Narcolepsy:0.89), (CALCIUM CARBONATEERGOCALCIFEROLRETINOL:Narcolepsy:0.82), (CALCIUM CARBONATEETIDRONATE DISODIUM:Narcolepsy:0.68), (CALCIUM PANTOTHENATE:Narcolepsy:0.19), (CALCIUM PANTOTHENATECUPRIC SULFATECYANOCOBALAMINFERROUS ASPARTO GLYCINATEFERROUS FUMARATEFOLIC ACIDMAGNESIUM SULFATENIACINAMIDEPYRIDOXINE HYDROCHLORIDE\RIBOFLAVIN\SODIUM ASCORBATETHIAMINE MONONITRATE\ZINC SULFATE:Narcolepsy:0.13), (CAMPHOR (SYNTHETIC)CAPSICUM OLEORESINMENTHOLMETHYL SALICYLATE:Narcolepsy:0.16), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:Narcolepsy:0.19), (CAMPHORDIPHENHYDRAMINE\ZINC OXIDE:Narcolepsy:0.62), (CANAKINUMAB:Narcolepsy:1.1), (CARBIDOPAENTACAPONE LEVODOPA:Narcolepsy:0.21), (CARBOMER:Narcolepsy:0.17), (CARBOMER 934GLYCERINMETHYLPARABENPROPYLPARABEN\SODIUM HYDROXIDE:Narcolepsy:0.38), (CARFILZOMIB:Narcolepsy:0.97), (CEFAZOLIN SODIUM:Narcolepsy:0.29), (CERIVASTATIN SODIUM:Narcolepsy:1.45), (CHLORAMPHENICOL:Narcolepsy:0.21), (CHLORMADINONE ACETATEETHINYL ESTRADIOL:Narcolepsy:0.37), (CHLOROTHIAZIDE:Narcolepsy:0.21), (CHLORPHENIRAMI NE MALEATEIBUPROFENPSEUDOEPHEDRINE HYDROCHLORIDE:Narcolepsy:1.11), (CHLORPROTHIXENE:Narcolepsy:0.56), (CHOLECA LCIFEROLGENISTEINZINC GLYCINATE:Narcolepsy:0.72), (CICLOPIROX OLAMINE:Narcolepsy:0.71), (CITRIC ACID MONOHYDRATE\POTASSIUM CITRATE:Narcolepsy:0.04), (CLIKSTAR:Narcolepsy:0.74), (CLOSTRIDIUM TETANI TOXOID ANTIGEN (FORMALDEHYDE INACTIVATED)DIPHTHERIAE TOXOID ANTIGEN (FORMALDEHYDE INACTIVATED):Narcolepsy:0.07), (CLOTRIMAZOLEHYDROCORTISONE:Narcolepsy:0.08), (CLOXAZOLAM:Narcolepsy:0.42), (CODEINE PHOSPHATEDICLOFENAC SODIUM:Narcolepsy:0.03), (COPPERINTRAUTERINE DEVICE:Narcolepsy:0.23), (COUMARINTROXERUTIN:Narcolepsy:0.11), (CROFELEMER:N arcolepsy:0.28), (CYANOCOBALAMINDICLOFENAC SODIUMPYRIDOXINE HYDROCHLORIDETHIAMINE MONONITRATE:Narcolepsy:0.61), (CYANOCOBALAMINPYRIDOXINE HYDROCHLORIDE\RIBOFLAVIN\SODIUM PANTOTHENATETHIAMINE HYDROCHLORIDEVITAMIN B COMPLEX:Narcolepsy:0.08), (DABIGATRAN:Narcolepsy:0.28), (DACLIZUMAB:Narcolepsy: 2.11), (DALTEPARIN:Narcolepsy:0.58), (DANTROLENE SODIUM:Narcolepsy:0.75), (DASABUVIROMBITASVIRPARITAPREVIRRITONAVIR:Na rcolepsy:1.16), (DEFLAZACORT:Narcolepsy:1.13), (DESOGESTREL:Narcolepsy:0.86), (DEUT ETRABENAZINE:Narcolepsy:0.3), (DEXAMETHASONENEOMYCINPOLYMYXIN B SULFATE:Narcolepsy:0.04), (DICHLORPHENAMIDE:Narcolepsy:0.78), (DICLOFENAC DIETHYLAMINE:Narcolepsy:0.06), (DIENOGEST:Narcolepsy:0.82), (DIHYDROCODEINE PHOSPHATE:Narcolepsy:0.14), (DIMETHYL SULFONEGLUCOSAMINE SULFATE:Narcolepsy:0.24), (DIMETHYL SULFOXIDE:Narcolepsy:1.52), (DIOSMIN:Narcolepsy:0.45), (DIPHTHERIA AND TETANUS TOXOIDS AND ACELLULAR PERTUSSIS VACCINE ADSORBED:Narcolepsy:0.4), (DIPYRONE:Narcolepsy:0.98), (DIPYRONEPROMETHAZINE HYDROCHLORIDE:Narcolepsy:1.78), (DOCETAXEL ANHYDROUS:Narcolepsy:0.12), (DOLASETRON MESYLATE:Narcolepsy:0.55), (DOTHIEPINDOTHIEPIN HYDROCHLORIDE:Narcolepsy:0.64), (DYCLONINE:Narcolepsy:0.47), (DYDROGESTERON EESTRADIOL:Narcolepsy:0.25), (EDOXABAN TOSYLATE:Narcolepsy:1.14), (ELTROMBOPAG OLAMINE:Narcolepsy:0.71), (ENTACAPONE:Narcolepsy:0.09), (ENZALUTAMIDE:Narcolep sy:0.41), (ERGOLOID MESYLATES:Narcolepsy:0.22), (ETHACRYNIC ACID:Narcolepsy:0.96), (ETHINYL ESTRADIOLGESTODENE:Narcolepsy:1.23), (ETHOSUXIMIDE:Narcolepsy:0.06), (EUCALY PTOLMENTHOLMETHYL SALICYLATETHYMOL:Narcolepsy:0.54), (FELBAMATE:Narcolepsy:0.32), (FENOTEROL HYDROBROMIDE:Narcolepsy:0.23), (FERROUS FUMARATEFOLIC ACIDVITAMIN B COMPLEX:Narcolepsy:0.44), (FESOTERODINE:Narcolepsy:0.06), (FLIBANSERIN:Narcoleps y:0.77), (FLUCLOXACILLIN:Narcolepsy:0.07), (FLUNARIZINE HYDROCHLORIDE:Narcolepsy:0.61), (FLUPENTIXOL DIHYDROCHLORIDEMELITRACEN HYDROCHLORIDE:Narcolepsy:0.19), (FLUTICASONE FUROATESALMETEROL XINAFOATE:Narcolepsy:1.33), (FOLIC ACIDIRON:Narcolepsy:0.3), (FOLIC ACIDIRONMINERALSVITAMINS:Narcolepsy:0.33), (FORMOTEROL:Narcolepsy:0.42), (F ORMOTEROL FUMARATEGLYCOPYRROLATE:Narcolepsy:0.25), (FORMOTEROL FUMARATE\MOMETASONE FUROATE:Narcolepsy:1.37), (FROVATRIPTAN:Narcolepsy:0.31), (FUROSEMIDEPOTASSIUM CHLORIDE:Narcolepsy:0.04), (GENISTEIN:Narcolepsy:0.52), (GLATIRAMER:Narcolepsy:0.6), (GLUCONATE:Narcolepsy:0.29), (GLUCOSAMINE HYDROCHLORIDE:Narcolepsy:0.3), (GLYCOPYRROLATEINDACATEROL MALEATE:Narcolepsy:0.77), (GLYCOPYRRONIUMINDACATEROL:Narcolepsy:0.88), (GO LD:Narcolepsy:0.15), (GOLDENSEAL:Narcolepsy:0.02), (GUAIFENESINPHENYLEPHRINE HYDROCHLORIDE:Narcolepsy:0.79), (HEPATITIS A VIRUS STRAIN HM175 ANTIGEN (FORMALDEHYDE INACTIVATED):Narcolepsy:0.17), (HEPATITIS B VIRUS VACCINE:Narcolepsy:0.17), (HERBALSHOMEOPATHICSGOLDENSEAL:Narcolepsy:1.05), (HERBALSVITIS VINIFERA SEED:Narcolepsy:1.05), (HERBSUNSPECIFIED INGREDIENT:Narcolepsy:0.32), (HIGH DENSITY POLYETHYLENE:Narcolepsy:0.86), (HUMAN COLOSTRUM:Narcolepsy:0.33), (HYDRAZINE SULFATE:Narcolepsy:0.37), (HYDROCHLORIC ACID:Narcolepsy:0.63), (HYDROCHLOROTHIAZIDEQUINAPRIL HYDROCHLORIDE:Narcolepsy:0.05), (HYDROCORTISONEMICONAZOLE:Narcolepsy:0.12), (HYDROCORTISONENEOMYCIN SULFATEPOLYMYXIN B SULFATE:Narcolepsy:0.3), (HYDROGEN PEROXIDE:Narcolepsy:0.15), (HYDROXOCOBALAMIN ACETATE:Narcolepsy:0.45), (HYPERICUM PERFORATUM:Narcolepsy:0.77), (IBRUTINIB:Narcolepsy:0.75), (ILOPROST:Narcolepsy:0.21), (IMIGLUCERASE:Narcolepsy:0.17), (INDACATEROL:Narcolepsy:0.4), (INFLIXIMAB-DYYB:Narcolepsy:0.69), (INFLUENZA A VIRUS A/BRISBANE/10/2010 (H1N1) ANTIGEN (MDCK CELL DERIVED, PROPIOLACTONE INACTIVATED)\INFLUENZA A VIRUS A/VICTORIA/361/2011 IVR-165 (H3N2) ANTIGEN (MDCK CELL DERIVED, PROPIOLACTONE INACTIVATED)\INFLUENZA B VIRUS B/WISCONSIN/1/2010) ANTIGEN (MDCK CELL DERIVED, PROPIOLACTONE INACTIVATED):Narcolepsy:0.36), (INSULIN BEEF:Narcolepsy:1.02), (INSULIN PORKINSULIN PURIFIED PORK:Narcolepsy:0.11), (INTERFERON GAMMA-1B:Narcolepsy:0.73), (IRON POLYMALTOSE:Narcolepsy:0.03), (ISOFLAVONES SOY:Narcolepsy:0.09), (ISOPROPYL ALCOHOLLEUPROLIDE ACETATENORETHINDRONE ACETATE:Narcolepsy:1.47), (ITRACONAZOLE\SODIUM CHLORIDE:Narcolepsy:0.38), (IVACAFTOR:Narcolepsy:0.42), (IXAZOMIB CITRATE:Narcolepsy:0.01), (JWH-018:Narcolepsy:0.32), (LACTOPEROXIDASE BOVINELYSOZYME HYDROCHLORIDE:Narcolepsy:0.52), (LANOLINMINERAL OIL:Narcolepsy:0.2), (LANREOTIDE ACETATE:Narcolepsy:0.21), (LAPATINIB DITOSYLATE:Narcolepsy:0.05), (LATANOPROSTTIMOLOL:Narcolepsy:0.41), (LAVENDE R OIL:Narcolepsy:0.38), (LETROZOLERIBOCICLIB:Narcolepsy:0.14), (LEVOBUNOLOL HYDROCHLORIDE:Narcolepsy:0.24), (LEVOMEFOLATE CALCIUM\METHYLCOBALAMINPYRIDOXAL 5-PHOSPHATE:Narcolepsy:0.37), (LOFEPRAMINE:Narcolepsy:0.56), (LOMITAPIDE MESYLATE:Narcolepsy:2.14), (LUTETIUM LU-177:Narcolepsy:0.39), (MAPROTILINE HYDROCHLORIDE:Narcolepsy:0.09), (RECUTITA:Narcolepsy:1.21), (MEBEVERINE:Narcolepsy:0), (MECLOFENAMATE SODIUM:Narcolepsy:0.05), (MECLOZINE DIHYDROCHLORIDE:Narcolepsy:1.17), (MEFLOQUINE HYDROCHLORIDE:Narcolepsy:0.08), (MELPERONE:Narcolepsy:0.01), (MENTHOLZINC OXIDE:Narcolepsy:0.88), (METFORMIN HYDROCHLORIDESAXAGLIPTIN:Narcolepsy:0.05), (METFORMIN HYDROCHLORIDEVILDAGLIPTIN:Narcolepsy:0.34), (METHACHOLINE CHLORIDE:Narcolepsy:0.01), (METHENAMINE MANDELATE:Narcolepsy:1.6), (METHSUXIMIDE:Narcolepsy:0.32), (METRELEPTIN:Narcol epsy:0.42), (METYROSINE:Narcolepsy:0.1), (MINERALSOMEGA-3 FATTY ACIDSVITAMINS:Narcolepsy:0.17), (MOEXIPRIL HYDROCHLORIDE:Narcolepsy:1.58), (MONOSODIUM GLUTAMATE:Narcolepsy:0.11), (MORNIFLUMATE:Narcolepsy:0.21), (NATURAL LATEX RUBBER:Narcolepsy:0.06), (NEBIVOLOL:Narcolepsy:0.15), (NIACINAMIDEPYRIDOXINE RIBOFLAVINTHIAMINE:Narcolepsy:0.4), (NIMESULIDE:Narcolepsy:0.21), (NIRAPARIB:N arcolepsy:0.71), (NOREPINEPHRINE BITARTRATE:Narcolepsy:0.13), (OLANZAPINE PAMOATE:Narcolepsy:0.03), (OLMESARTAN:Narcolepsy:0.14), (OMEGA-6 FATTY ACIDS:Narcolepsy:0.94), (OPIPRAMOL:Narcolepsy:0.3), (OXYMETAZOLINE HYDROCHLORIDEPSEUDOEPHEDRINE HYDROCHLORIDE:Narcolepsy:0.21), (OXYQUINOLINE SULFATE\SODIUM LAURYL SULFATE:Narcolepsy:1), (PALONOSETRON HYDROCHLORIDE:Narcolepsy:0.01), (PAMIDRONATE DISODIUM:Narcolepsy:0.98), (PANCRELIPASEPANCRELIPASE AMYLASEPANCRELIPASE LIPASEPANCRELIPASE PROTEASE:Narcolepsy:0.09), (PANTOPRAZOLE MAGNESIUM:Narcolepsy:2.32), (PAPAYA:Narcolepsy:1.64), (PARATHYROID HORMONE:Narcolepsy:0.8), (PARSLEY:Narcolepsy:0.59), (PATIROMER:Narcolepsy:1.13), (P AZOPANIB HYDROCHLORIDE:Narcolepsy:0.85), (PENBUTOLOL SULFATE:Narcolepsy:0.07), (PENICILLIN V:Narcolepsy:0.33), (PENTOBARBITAL SODIUM:Narcolepsy:0.27), (PHENOBARBITAL SODIUM:Narcolepsy:0.03), (PINAVERIUM BROMIDE:Narcolepsy:0.43), (PIRFENIDONE:Narcolepsy:0.21), (PIZOTYLINE:Narcolepsy:0.06), (PLECANATIDE:Narcolepsy:0.34), (PODOFILOX:Narcolepsy:0.34), (POLYETHYLENE GLYCOL 400:Narcolepsy:0.51), (PONATINIB:Narcolepsy:1.28), (PREDNISOLONE ACETATESULFACETAMIDE SODIUM:Narcolepsy:0.65), (PREDNISOLONEPREDNISOLONE ACETATEPREDNISOLONE SODIUM SUCCINATE:Narcolepsy:0.05), (PROBENECID:Narcolepsy:0.04), (PROPATYL NITRATE:Narcolepsy:0.3), (PROPIOMAZINE:Narcolepsy:0.01), (PROSTATE:Narcolepsy:0.06), (QUINAGOLIDE HYDROCHLORIDE:Narcolepsy:0.16), (QUINIDINE GLUCONATE:Narcolepsy:1.12), (REBOXETINE:Narcolepsy:0.3), (REISHI:Narcolepsy:0), (RE SLIZUMAB:Narcolepsy:0.04), (RILPIVIRINE HYDROCHLORIDE:Narcolepsy:0.14), (ROFECOXIB:Narcolepsy:1.05), (RUFINAMIDE:Narco lepsy:0.1), (SAPROPTERIN DIHYDROCHLORIDE:Narcolepsy:0.3), (SERTINDOLE:Narcolepsy:0.48), (SIPULEUCEL-T:Narcolepsy:0.25), (SITAGLIPTIN:Narcolepsy:0.42), (SODIUM CITRATE:Narcolepsy:0.19), (SODIUM HYPOCHLORITE:Narcolepsy:0.37), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Narcolepsy:0.22), (SOFOSBUVIRVELPATASVIR:Narcolepsy:0.93), (STR ONTIUM:Narcolepsy:0.53), (TEDUGLUTIDE:Narcolepsy:0.38), (TEDUGLUTIDE\WATER:Na rcolepsy:1.18), (TEGASEROD:Narcolepsy:0.32), (TELOTRISTAT ETHYL:Narcolepsy:0.15), (TESAMORELIN:Narcolepsy:0.86), (TESTOSTERONE ENANTHATE:Narcolepsy:0.01), (TESTOSTERONE UNDECANOATE:Narcolepsy:0.47), (THALIDOMIDE:Narcolepsy:0.73), (THEOPHYLLINE:N arcolepsy:1.16), (THIOTHIXENE:Narcolepsy:0.77), (TIBOLONE:Narcolepsy:1.45), (TIMOLOL MALEATETRAVOPROST:Narcolepsy:0.3), (TIOCONAZOLE:Narcolepsy:0.73), (TIOPRONI N:Narcolepsy:0.63), (TIOTROPIUM:Narcolepsy:0.84), (TOLMETIN:Narcolepsy:0.53), (TREPR OSTINIL DIOLAMINE:Narcolepsy:0.07), (TRIENTINE HYDROCHLORIDE:Narcolepsy:0.54), (TRIMETHADIONE:Narcolepsy:0.54), (TROSPIUM:Na rcolepsy:0.49), (VALBENAZINE:Narcolepsy:0.29), (VALDECOXIB:Narcolepsy:1.1), (VALRU BICIN:Narcolepsy:0.45), (VISMODEGIB:Narcolepsy:0.4), (VORAPAXAR SULFATE:Narcolepsy:0.04), (WHEAT GLUTEN:Narcolepsy:0.02), (.ALPHA.1-PROTEINASE INHIBITOR HUMAN:Nasal congestion:1.42), (ABIRATERONE ACETATE:Nasal congestion:0.13), (ACETAZOLAMIDE:Nasal congestion:1.08), (AGALSIDASE BETA:Nasal congestion:0.01), (ALBIGLUTIDE:Nasal congestion:0.6), (ALEMTUZUMAB:Nasal congestion:0.07), (ALISKIREN HEMIFUMARATE:Nasal congestion:0.44), (AMISULPRIDE:Nasal congestion:0.44), (AMLODIPINE BESYLATEHYDROCHLOROTHIAZIDEOLMESARTAN MEDOXOMIL:Nasal congestion:0.33), (AMLODIPINEVALSARTAN:Nasal congestion:0.16), (AMPICILLIN:Nasal congestion:0.11), (ATOVAQUONE:Nasal congestion:0.1), (BECLOMETHASONEFORMOTEROL:Nasal congestion:0.02), (BENAZEPRILBENAZEPRIL HYDROCHLORIDE:Nasal congestion:0.81), (BENDAMUSTINE HYDROCHLORIDE:Nasal congestion:0.17), (BETAMETHASONE DIPROPIONATECALCIPOTRIENECALCIPOTRIENE HYDRATE:Nasal congestion:0.13), (BRINZOLAMIDE:Nasal congestion:0.89), (BROMAZEPAM:Nasal congestion:0.2), (BUTYLSCOPOLAMINE BROMIDE:Nasal congestion:0.13), (CALCITONIN:Nasal congestion:0.11), (CALCIUM ACETATE:Nasal congestion:1.25), (CALCIUM\MAGNESIUM:Nasal congestion:0.55), (CALCIUM\MAGNESIUM\ZINC:Nasal congestion:0.11), (CANAGLIFLOZINMETFORMIN HYDROCHLORIDE:Nasal congestion:0.46), (CANAKINUMAB:Nasal congestion:0.62), (CANDESARTAN CILEXETILHYDROCHLOROTHIAZIDE:Nasal congestion:0.99), (CARVEDILOL PHOSPHATE:Nasal congestion:1.27), (CEFOTAXIME SODIUM:Nasal congestion:0.32), (CEVIMELINE HYDROCHLORIDE:Nasal congestion:0.15), (CHLORAMBUCIL:Nasal congestion:0.11), (CILASTATIN SODIUMIMIPENEM:Nasal congestion:0.69), (CYPROHEPTADINE:Nasal congestion:0.75), (DACLIZUMAB:Nasal congestion:1.18), (DALTEPARIN:Nasal congestion:0.39), (DALTEPARIN SODIUM:Nasal congestion:0.07), (DASATINIB:Nasal congestion:1.76), (DEFLAZACORT:Nasal congestion:0.57), (DICLOFENAC SODIUMMISOPROSTOL:Nasal congestion:0.04), (DIPYRONE:Nasal congestion:0.34), (DOLASETRON MESYLATE:Nasal congestion:0.13), (DORZOLAMIDE HYDROCHLORIDE:Nasal congestion:0.08), (DUTASTERIDETAMSULOSIN HYDROCHLORIDE:Nasal congestion:0.16), (EDOXABAN TOSYLATE:Nasal congestion:0.45), (ELETRIPTAN HYDROBROMIDE:Nasal congestion:1.61), (ELOTUZUMAB:Nasal congestion:0.14), (EMPAGLIFLOZINLINAGLIPTIN:Nasal congestion:0.67), (EPIRUBICIN:Nasal congestion:0.38), (ERYTHROPOIETIN:Nasal congestion:0.78), (ESTRONESUS SCROFA OVARY:Nasal congestion:0.35), (EVOLOCUMAB:Nasal congestion:1.53), (FELODIPINE:Nasal congestion:0.07), (FORMOTEROL:Nasal congestion:0.46), (FORMOTEROL FUMARATE\MOMETASONE FUROATE:Nasal congestion:1.19), (FULVESTRANT:Nasal congestion:0.5), (FUSIDIC ACID:Nasal congestion:0.29), (GIMERACILOTERACILTEGAFUR:Nasal congestion:0.18), (GLYCOPYRRONIUMINDACATEROL:Nasal congestion:0.69), (GUANFACINE:Nasal congestion:0.25), (GUANFACINE HYDROCHLORIDE:Nasal congestion:0.19), (HYDROCODONE BITARTRATEIBUPROFEN:Nasal congestion:0.26), (HYDROCORTISONE ACETATE:Nasal congestion:0.36), (ILOPROST:Nasal congestion:0.74), (IMATINIB MESYLATE:Nasal congestion:0.82), (IMIPRAMINE HYDROCHLORIDE:Nasal congestion:0.72), (INDACATEROL:Nasal congestion:0.3), (INDAPAMIDEPERINDOPRIL:Nasal congestion:0.28), (INFLIXIMAB-DYYB:Nasal congestion:0.08), (INSULIN GLULISINE:Nasal congestion:0.03), (INTERFERON GAMMA-1B:Nasal congestion:0.4), (IRINOTECAN HYDROCHLORIDE:Nasal congestion:0.47), (IVACAFTORLUMACAFTOR:Nasal congestion:0.39), (LAPATINIB DITOSYLATE:Nasal congestion:0.21), (LERCANIDIPINE:Nasal congestion:0.52), (LOMITAPIDE MESYLATE:Nasal congestion:0.53), (MAGNESIUM GLUCONATE:Nasal congestion:0.1), (MEGESTROL:Nasal congestion:0.01), (MEPOLIZUMAB:Nasal congestion:0.88), (METFORMIN HYDROCHLORIDEVILDAGLIPTIN:Nasal congestion:0.08), (METHENAMINE HIPPURATE:Nasal congestion:1.36), (MILK THISTLE:Nasal congestion:0.05), (MINOCYCLINEMINOCYCLINE HYDROCHLORIDE:Nasal congestion:1.12), (NALOXONE HYDROCHLORIDEOXYCODONE HYDROCHLORIDE:Nasal congestion:1.02), (NALTREXONE:Nasal congestion:0.04), (NORETHINDRONE:Nasal congestion:0.37), (OSELTAMIVIR:Nasal congestion:0.31), (PAZOPANIB HYDROCHLORIDE:Nasal congestion:0.57), (PEGINTERFERON BETA-1A:Nasal congestion:0.29), (PEMETREXED:Nasal congestion:0.15), (PENTOSAN POLYSULFATE SODIUM:Nasal congestion:0.4), (PENTOXIFYLLINE:Nasal congestion:0.13), (PERINDOPRIL:Nasal congestion:0.5), (PHENOBARBITAL:Nasal congestion:1.1), (PONATINIB:Nasal congestion:0.86), (PRAZOSIN HYDROCHLORIDE:Nasal congestion:0.3), (PREDNISOLONE SODIUM PHOSPHATE:Nasal congestion:0.27), (PREDNISOLONEPREDNISOLONE ACETATEPREDNISOLONE SODIUM SUCCINATE:Nasal congestion:0.34), (RABEPRAZOLE:Nasal congestion:0.93), (RADIATION THERAPY:Nasal congestion:0.23), (REGORAFENIB:Nasal congestion:0.26), (RIVASTIGMINE:Nasal congestion:0.08), (RIZATRIPTAN:Nasal congestion:0.09), (SULFAMETHOXAZOLE:Nasal congestion:1.2), (TETRACYCLINE:Nasal congestion:0.97), (THEOPHYLLINE:Nasal congestion:0.24), (TIOTROPIUM:Nasal congestion:0.68), (TRASTUZUMAB:Nasal congestion:0.3), (VORTIOXETINE HYDROBROMIDE:Nasal congestion:0.04), (ZAFIRLUKAST:Nasal congestion:0.59), (ZILEUTON:Nasal congestion:0.47), (NAPROXEN:Nasal discomfort:0.06), (BUMETANIDE:Nasal disorder:0.06), (CIPROFLOXACIN HYDROCHLORIDE:Nasal disorder:0.02), (DALFAMPRIDINE:Nasal disorder:0.07), (DIPHENHYDRAMINE:Nasal disorder:0.38), (DOCUSATE SODIUM:Nasal disorder:0.14), (EVOLOCUMAB:Nasal disorder:0.21), (FERROUS SULFATEFERROUS SULFATE, DRIED:Nasal disorder:0.2), (ILOPROST:Nasal disorder:0.05), (INSULIN ASPART:Nasal disorder:0.09), (IRON:Nasal disorder:0.23), (LOVASTATIN:Nasal disorder:0.38), (PANTOPRAZOLE:Nasal disorder:0.2), (PRAVASTATIN SODIUM:Nasal disorder:0.28), (RAMIPRIL:Nasal disorder:0.2), (RISEDRONATE SODIUM:Nasal disorder:0.27), (TAMSULOSIN:Nasal disorder:0.26), (TERIFLUNOMIDE:Nasal disorder:0.06), (VERAPAMIL HYDROCHLORIDE:Nasal disorder:0.38), (ACETAMINOPHENBUTALBITALCAFFEINE:Nasal dryness:0.27), (ACETAMINOPHENCODEINE PHOSPHATE:Nasal dryness:0.36), (ANASTROZOLE:Nasal dryness:0.12), (BISACODYL OR DOCUSATE SODIUM:Nasal dryness:0.01), (BISOPROLOL FUMARATE:Nasal dryness:0.02), (BUSPIRONE HYDROCHLORIDE:Nasal dryness:0.36), (CALCIUM CARBONATECHOLECALCIFEROL:Nasal dryness:0.14), (CANDESARTAN:Nasal dryness:0), (CARFILZOMIB:Nasal dryness:0.3), (CERTOLIZUMAB PEGOL:Nasal dryness:0.4), (CLINDAMYCINCLINDAMYCIN PHOSPHATE:Nasal dryness:0.92), (COLCHICINE:Nasal dryness:0.28), (DASATINIB:Nasal dryness:0.15), (DULOXETINE:Nasal dryness:0.14), (ENALAPRIL:Nasal dryness:1.06), (EVOLOCUMAB:Nasal dryness:0.33), (EZETIMIBE:Nasal dryness:0.14), (FERROUS SULFATEFERROUS SULFATE, DRIED:Nasal dryness:0.75), (FINGOLIMOD HYDROCHLORIDE:Nasal dryness:0.24), (ILOPROST:Nasal dryness:0.41), (LIDOCAINEPRILOCAINE:Nasal dryness:0.04), (METAXALONE:Nasal dryness:0.07), (NEBIVOLOL:Nasal dryness:0.07), (NIFEDIPINE:Nasal dryness:0.03), (OXCARBAZEPINE:Nasal dryness:0.27), (PRIMIDONE:Nasal dryness:0.22), (PROMETHAZINEPROMETHAZINE HYDROCHLORIDE:Nasal dryness:0.38), (RAMIPRIL:Nasal dryness:0.77), (RANOLAZINE:Nasal dryness:0.59), (TERIFLUNOMIDE:Nasal dryness:0.71), (TRIAMTERENE:Nasal dryness:0.02), (VARENICLINE TARTRATE:Nasal dryness:0.07), (.ALPHA.1-PROTEINASE INHIBITOR HUMAN:Nasopharyngitis:1.21), (ACARBOSE:Nasopharyngitis:0.86), (ACETAMINOPHENAP RONALIDECAFFEINEPROPYPHENAZONE:Nasopharyngitis:0.44), (ACETAMINOPHEN BROMISOVALDIHYDROCODEINE PHOSPHATEDIPHENHYDRAMINE SALICYLATEDYPHYLLINEMETHYLEPHEDRINE HYDROCHLORIDE, (+/−)-:Nasopharyngitis:0.3), (ACETAMINOPHENBUTALBITALCAFFEINE:Nasopharyngitis: 2.05), (ACETAMINOPHEN\CAFFEINE:Nasopharyngitis:0.05), (ACETAMINOPHENOXYCO DONEOXYCODONE HYDROCHLORIDE:Nasopharyngitis:0.93), (ACETAMINOPHENPROPOXYPHENE HYDROCHLORIDE:Nasopharyngitis:0.24), (ACETAMINOPHENPROPOXYPHENE NAPSYLATE:Nasopharyngitis:0.57), (ACTIVATED CHARCOAL:Nasopharyngitis:0.51), (ADENINE:Nasopharyngitis:0.61), (AGALSIDASE BETA:Nasopharyngitis:0.53), (ALEMTUZUMAB:Nasopharyngitis:0.45), (ALFUZOSIN HYDROCHLORIDE:Nasopharyngitis:0.2), (ALISKIREN HEMIFUMARATE:Nasopharyngitis:0.25), (AMILORIDE HYDROCHLORIDEHYDROCHLOROTHIAZIDE:Nasopharyngitis:0.2), (AMISULPRIDE:Na sopharyngitis:0.74), (AMLODIPINE BESYLATEATORVASTATIN CALCIUM:Nasopharyngitis:0.3), (AMLODIPINE BESYLATEHYDROCHLOROTHIAZIDEVALSARTAN:Nasopharyngitis:0.14), (AMLODIPINE BESYLATETELMISARTAN:Nasopharyngitis:0.84), (AMLODIPINEVALSARTAN:Nasophar yngitis:0.3), (AMMONIUM GLYCYRRHIZATE:Nasopharyngitis:0.04), (ANAGLIPTIN:Nasopharyngitis:0.16), (ARIPIPRA ZOLE LAUROXIL:Nasopharyngitis:0.02), (ASPIRINLANSOPRAZOLE:Nasopharyngitis:0.2), (ASPI RINMAGNESIUM OXIDE:Nasopharyngitis:0.13), (ATOVAQUONE:Nasopharyngitis:0.37), (AZELNIDIPINEOL MESARTAN MEDOXOMIL:Nasopharyngitis:0.26), (AZTREONAM:Nasopharyngitis:0.01), (BACITRACIN: Nasopharyngitis:0.19), (BALSALAZIDE DISODIUM:Nasopharyngitis:0.1), (BECLOMETHASONEFORMOTEROL:Nasopharyngitis:0.33), (BENZTROPINE MESYLATE:Nasopharyngitis:0.16), (BERAPROST:Nasopharyngitis:0.07), (BETAMETHASON E BUTYRATE PROPIONATE:Nasopharyngitis:0.43), (BETAMETHASONE DIPROPIONATECLOTRIMAZOLE:Nasopharyngitis:0), (BETAMETHASONE SODIUM PHOSPHATENEOMYCIN SULFATE:Nasopharyngitis:0.42), (BRIMONIDINE TARTRATETIMOLOL MALEATE:Nasopharyngitis:0.29), (BUPRENORPHINE HYDROCHLORIDE:Nasopharyngitis:1.59), (BUPRENORPHINE HYDROCHLORIDENALOXONE HYDROCHLORIDE:Nasopharyngitis:1.53), (BUPRENORPHINENALOXONE:Nasopharyngiti s:0.12), (CALCITONIN:Nasopharyngitis:0.36), (CALCITONIN SALMON:Nasopharyngitis:0.94), (CALCIUM CARBONATEERGOCALCIFEROLRETINOL:Nasopharyngitis:0.39), (CALCIUMMAGNES IUM:Nasopharyngitis:0.63), (CALCIUM\MAGNESIUM\ZINC:Nasopharyngitis:0.23), (CAMPH ORMENTHOLMETHYL SALICYLATE:Nasopharyngitis:0.99), (CANAGLIFLOZINMETFORMIN HYDROCHLORIDE:Nasopharyngitis:0.58), (CANAKINUMAB:Nasopharyngitis:1.27), (CARBI MAZOLE:Nasopharyngitis:0.27), (CARVEDILOL PHOSPHATE:Nasopharyngitis:1.31), (CASANTHRANOLDOCUSATE SODIUM:Nasopharyngitis:0.03), (CEFOTIAM HYDROCHLORIDE:Nasopharyngitis:0.04), (CEFTAZIDIME:Nasopharyngitis:0.1), (CELLULA SEDIASTASELIPASE:Nasopharyngitis:0.64), (CEPHARANTHINE:Nasopharyngitis:0.68), (C ETUXIMAB:Nasopharyngitis:0.78), (CHLORAMBUCIL:Nasopharyngitis:0.06), (CHLORDIAZ EPOXIDE HYDROCHLORIDECLIDINIUM BROMIDE:Nasopharyngitis:0.19), (CHLORPROMAZINEPROMETHAZINE:Nasopharyngitis:0), (CINACALCET HYDROCHLORIDE:Nasopharyngitis:0.17), (CLOBAZAM:Nasopharyngitis:0.66), (CLONIDIN E HYDROCHLORIDE:Nasopharyngitis:0.66), (COD LIVER OIL:Nasopharyngitis:0.52), (COLCHICINE:Nasopharyngitis:1.12), (COLESTILAN CHLORIDE:Nasopharyngitis:0.48), (CORTICOTROPIN:Nasopharyngitis:0.66), (CYPROHEPT ADINE:Nasopharyngitis:0.37), (DABIGATRAN:Nasopharyngitis:0.28), (DACLIZUMAB:Nasop haryngitis:0.91), (DANAZOL:Nasopharyngitis:0.09), (DANTROLENE SODIUM:Nasopharyngitis:0.09), (DEVICE:Nasopharyngitis:0.52), (DILAZEP:Nasopharyngitis:20.15), (DIPHENIDOL HYDROCHLORIDE:Nasopharyngitis:1.03), (DOXORUBICIN HYDROCHLORIDE:Nasopharyngitis:0.13), (DRONABINOL:Nasopharyngitis:1.24), (DUTAST ERIDETAMSULOSIN HYDROCHLORIDE:Nasopharyngitis:0.41), (ECONAZOLE:Nasopharyngitis:0.57), (EDARAV ONE:Nasopharyngitis:0.16), (EDOXABAN TOSYLATE:Nasopharyngitis:1.41), (ELCATONIN:Nasopharyngitis:0.08), (ELOTUZUMAB:Na sopharyngitis:0.42), (EMPAGLIFLOZINLINAGLIPTIN:Nasopharyngitis:0.06), (ERYTHROM YCIN LACTOBIONATE:Nasopharyngitis:0.45), (ESTAZOLAM:Nasopharyngitis:0.15), (ESTROGEN S:Nasopharyngitis:0.18), (ESTRONESUS SCROFA OVARY:Nasopharyngitis:0.27), (ETORICOXIB:Nasopharyngitis:0.97), (EVOLOCUMAB:Naso pharyngitis:1.82), (EZETIMIBESIMVASTATIN:Nasopharyngitis:1.2), (FAROPENEM SODIUM:Nasopharyngitis:0.7), (FENOFIBRIC ACID:Nasopharyngitis:0.26), (FENOTEROL HYDROBROMIDE:Nasopharyngitis:0.4), (FENOTEROL HYDROBROMIDEIPRATROPIUM BROMIDE:Nasopharyngitis:0.65), (FLUDROCORTISONE ACETATE:Nasopharyngitis:0.74), (FLUOCINOLONE ACETONIDE:Nasopharyngitis:0.2), (FLURAZEPAMFLURAZEPAM HYDROCHLORIDE:Nasopharyngitis:0.1), (FLUTICASONE PROPIONATEFORMOTEROL FUMARATE:Nasopharyngitis:0.71), (FOLATE SODIUM:Nasopharyngitis:0.12), (FONDAPARINUX SODIUM:Nasopharyngitis:0.14), (FORMOTEROL:Nasopharyngitis:0.69), (FOSFOMYCIN CALCIUM:Nasopharyngitis:0.89), (FOSPHENYTOINFOSPHENYTOIN SODIUM:Nasopharyngitis:0.01), (FULVESTRANT:Nasopharyngitis:0.5), (FURSULTIAMINE: Nasopharyngitis:0.54), (FUSIDIC ACID:Nasopharyngitis:0.85), (GEMCITABINE HYDROCHLORIDE:Nasopharyngitis:0.01), (GIMERACILOTERACILTEGAFUR:Nasophary ngitis:0.92), (GLYCOPYRROLATEINDACATEROL MALEATE:Nasopharyngitis:0.38), (HEPARIN CALCIUM:Nasopharyngitis:0.49), (HYDROCODONE BITARTRATEIBUPROFEN:Nasopharyngitis:0.28), (HYDROCORTISONE PROBUTATE:Nasopharyngitis:0.57), (HYDROXYCHLOROQUINE DIPHOSPHATE:Nasopharyngitis:0.57), (HYOSCYAMINE:Nasopharyngitis:0.77), (ILOPROST: Nasopharyngitis:0.62), (IMIDAPRIL HYDROCHLORIDE:Nasopharyngitis:0.02), (IMIPRAMINE HYDROCHLORIDE:Nasopharyngitis:1.29), (INFLIXIMAB-DYYB:Nasopharyngitis:0.56), (INSULIN BEEF:Nasopharyngitis:0.17), (INTERFERON:Nasopharyngitis:0.29), (IRINOTECAN HYDROCHLORIDE:Nasopharyngitis:0.45), (ITOPRIDE:Nasopharyngitis:0.09), (IXAZOMIB:N asopharyngitis:0.27), (LAFUTIDINE:Nasopharyngitis:1.14), (LANDIOLOL HYDROCHLORIDE:Nasopharyngitis:0.12), (LAPATINIB DITOSYLATE:Nasopharyngitis:0.2), (LERCANIDIPINE:Nasopharyngitis:1.24), (LERCANIDIP INE HYDROCHLORIDE:Nasopharyngitis:0.98), (LEVOCARNITINE HYDROCHLORIDE:Nasopharyngitis:1.12), (LEVOLEUCOVORIN CALCIUM:Nasopharyngitis:0.38), (LIDOCAINEPRILOCAINE:Nasopharyngitis:0.84), (LIOTH YRONINE SODIUM:Nasopharyngitis:0.38), (MEPOLIZUMAB:Nasopharyngitis:1.1), (METFORMIN HYDROCHLORIDEVILDAGLIPTIN:Nasopharyngitis:0.44), (METHENAMINE HIPPURATE:Nasopharyngitis:1.29), (MINOCYCLINEMINOCYCLINE HYDROCHLORIDE:Nasopharyngitis:1.73), (MITIGLINIDE:Nasopharyngitis:0.84), (MITIGLIN IDE CALCIUM HYDRATEVOGLIBOSE:Nasopharyngitis:0.32), (MOEXIPRIL HYDROCHLORIDE:Nasopharyngitis:0.41), (MORPHINE SULFATE\NALTREXONE HYDROCHLORIDE:Nasopharyngitis:0.02), (NALOXONE HYDROCHLORIDETILIDINE HYDROCHLORIDE:Nasopharyngitis:0.3), (OLMESARTAN:Nasopharyngitis:0.37), (OSELTA MIVIR:Nasopharyngitis:0.16), (OSPEMIFENE:Nasopharyngitis:0.11), (PEGINTERFERON BETA-1A:Nasopharyngitis:1.07), (PILOCARPINE:Nasopharyngitis:0.4), (PIMECROLIMUS:Nasophary ngitis:0.38), (PIMOBENDAN:Nasopharyngitis:0.84), (PIROXICAM:Nasopharyngitis:0.68), (PNE UMOCOCCAL VACCINE, POLYVALENT 23:Nasopharyngitis:0.24), (POLYSTYRENE:Nasopharyngitis:0.02), (PONATINIB:Nasopharyngi tis:0.52), (POTASSIUM IODIDE:Nasopharyngitis:0.31), (PREDNISOLONE SODIUM PHOSPHATE:Nasopharyngitis:0.12), (PROPIVERINE:Nasopharyngitis:0.19), (PROTRIPTYLINE HYDROCHLORIDE:Nasopharyngitis:0.02), (QUINACRINE:Nasopharyngitis:0.4), (RADIATION THERAPY:Nasopharyngitis:0.19), (RAMUCIRUMAB:Nasopharyngitis:0.35), (RANOLAZINE: Nasopharyngitis:1.6), (REGORAFENIB:Nasopharyngitis:0.34), (RETINOL:Nasopharyngitis:0.32), (RIFAXIMIN:Nasopharyngitis:0.24), (RILPIVIRINE HYDROCHLORIDE:Nasopharyngitis:0.14), (RIOCIGUAT:Nasopharyngitis:0.1), (RIVASTIGM INE TARTRATE:Nasopharyngitis:0.4), (ROPINIROLE:Nasopharyngitis:0.79), (SACUBITRILVAL SARTAN:Nasopharyngitis:0.89), (SARPOGRELATE HYDROCHLORIDE:Nasopharyngitis:0.66), (SERRAPEPTASE:Nasopharyngitis:0.5), (SEVELA MER CARBONATE:Nasopharyngitis:0.07), (SIVELESTAT SODIUM:Nasopharyngitis:0.01), (SODIUM FLUORIDE:Nasopharyngitis:0.66), (SULINDAC:Nasopharyngitis:0.1), (SUPLATAST TOSILATE:Nasopharyngitis:0.97), (TAFLUPROST:Nasopharyngitis:0.1), (TESTOSTERONE UNDECANOATE:Nasopharyngitis:0.25), (TETRACYCLINE:Nasopharyngitis:1.23), (TINZAPA RIN:Nasopharyngitis:0.23), (TIOTROPIUM:Nasopharyngitis:0.97), (TOSUFLOXACIN TOSYLATE:Nasopharyngitis:0.31), (TRANDOLAPRIL:Nasopharyngitis:0.25), (TROSPIUM CHLORIDE:Nasopharyngitis:0.28), (TROXIPIDE:Nasopharyngitis:0.21), (UMECLIDINIUM BROMIDE:Nasopharyngitis:0.63), (VANCOMYCIN HYDROCHLORIDE:Nasopharyngitis:0.85), (VILAZODONE HYDROCHLORIDE:Nasopharyngitis:0.92), (VINORELBINE TARTRATE:Nasopharyngitis:0.05), (VONOPRAZAN:Nasopharyngitis:0.29), (VONOPRAZAN FUMURATE:Nasopharyngitis:0.46), (WARFARIN POTASSIUM:Nasopharyngitis:1.19), (YEAST:Nasopharyngitis:0.23), (ZAFIRLUKAST:Nasoph aryngitis:0.65), (ZIPRASIDONE HYDROCHLORIDEZIPRASIDONE MESYLATE:Nasopharyngitis:0.22), (.ALPHA.-TOCOPHEROLVITAMINS:Nausea:1.48), (.BETA.-CAROTENE:Nausea:0.85), ((1-743)-(1638-2332)-BLOOD-COAGULATION FACTOR VIII (SYNTHETIC HUMAN) FUSION PROTEIN WITH IMMUNOGLOBULIN G1 (SYNTHETIC HUMAN FC DOMAIN FRAGMENT), (1444-6′), (1447-9′)-BIS(DISULFIDE) WITH IMMUNOGLOBULIN G1 (SYNTHETIC HUMAN FC DOMAIN FRAGMENT):Nausea:0.04), (ABOBOTULINUMTOXINA:Nausea:1.08), (ACAMPROSATE:N ausea:0.46), (ACEMETACIN:Nausea:0.25), (ACETAMINOPHENAPRONALIDECAFFEINE PROPYPHENAZONE:Nausea:0.05), (ACETAMINOPHENBUTYLSCOPOLAMINE BROMIDE:Nausea:0.46), (ACETAMINOPHEN\CAFFEINECARISOPRODOLDICLOFENA C:Nausea:0.5), (ACETAMINOPHEN\CAFFEINECARISOPRODOLDICLOFENAC SODIUM:Nausea:0.44), (ACETAMINOPHENCHLORPHENIRAMINE MALEATEPSEUDOEPHEDRINE HYDROCHLORIDE:Nausea:0.85), (ACETOHYDROXAMIC ACID:Nausea:0.07), (ACLARUBICIN HYDROCHLORIDE:Nausea:1.03), (ACLIDINIUM BROMIDEFORMOTEROL FUMARATE:Nausea:0.36), (ACRIVASTINEPSEUDOEPHEDRINE HYDROCHLORIDE:Nausea:0.89), (ACTIVATED CHARCOAL:Nausea:1.23), (ADEMETIONINE SULFATE TOSILATE:Nausea:1.02), (ADENINE:Nausea:0.41), (AJMALINE:Nausea:1.1), (ALANINEAR GININEASPARTIC ACIDGLUTAMIC ACIDGLYCINEHISTIDINEISOLEUCINELEUCINELYSINE ACETATEMETHIONINEPHENYLALANINEPROLINESERINETHREONINETRYPTOP HANTYROSINEVALINE:Nausea:0.39), (ALBUTEROLGUAIFENESIN:Nausea:0.92), (ALB UTEROLIPRATROPIUM BROMIDE:Nausea:0.67), (ALFALFA:Nausea:0.38), (ALGELDRATE:Nausea:0.06), (ALISKIRE N:Nausea:1.49), (ALISKIREN HEMIFUMARATEVALSARTAN:Nausea:0.29), (ALISKIRENHYDROCHLOROTHIAZIDE: Nausea:0.14), (ALLANTOINBENZOCAINECAMPHOR (NATURAL)DIMETHICONEMENTHOL:Nausea:0.01), (ALOGLIPTIN:Nausea:0.37), (ALU MINUM HYDROXIDEDIMETHICONEDIPHENHYDRAMINEHYDROXIDE IONLIDOCAINE:Nausea:0.9), (ALUMINUM SILICATE:Nausea:0), (AMBROXOLCLENBUTEROL:Nausea:0.99), (AMINO ACIDSCARBOHYDRATESMINERALSSOYBEAN OILVITAMINS:Nausea:1.36), (AMINOPHYLLINEQUININE SULFATE:Nausea:0.16), (AMLODIPINE BESYLATECANDESARTAN CILEXETIL:Nausea:0.15), (AMLODIPINE BESYLATEIRBESARTAN:Nausea:0.93), (AMLODIPINE MALEATE:Nausea:0.33), (AMLODIPINEATORVASTATIN:Nausea:0.06), (AMLODIPINEL OSARTAN:Nausea:0.06), (AMOBARBITAL SODIUM:Nausea:0.1), (AMOXICILLIN SODIUM:Nausea:0.56), (AMPHETAMINE SULFATE:Nausea:0.04), (AMPICILLIN SODIUM:Nausea:2.06), (ANAGRELIDE:Nausea:1.03), (ANTIHEMOPHILIC FACTOR, HUMAN RECOMBINANT RESIDUES 743-1636 DELETED:Nausea:0.11), (ARCTOSTAPHYLOS UVA-URSI LEAFDIOICA:Nausea:0.53), (ARIPIPRAZOLE LAUROXIL:Nausea:0.47), (ARSENIC TRIOXIDE:Nausea:1.24), (ASCORBIC ACID/CHROMIUM/SELENIUM/VANADYL SULFATE:Nausea:1.01), (ASCORBIC ACIDCHONDROITIN SULFATE (BOVINE)GLUCOSAMINE SULFATEMANGANESE:Nausea:0.06), (ASCORBIC ACIDFERROUS SULFATEFOLIC ACIDPYRIDOXINE HYDROCHLORIDE\RIBOFLAVINTHIAMINE MONONITRATE:Nausea:0.79), (ASCORBIC ACIDMINERALSVITAMINS:Nausea:0.11), (ASCORBIC ACIDVITAMIN B COMPLEX:Nausea:0.3), (ASENAPINE:Nausea:0.04), (ASPIRIN\CAFFEINESALICYLAMIDE: Nausea:0.92), (ATOMOXETINE:Nausea:0.28), (ATRACURIUM BESYLATE:Nausea:1.02), (ATROPINE SULFATEDIFENOXIN HYDROCHLORIDE:Nausea:0.1), (AVANAFIL:Nausea:0.14), (AVOBENZONEHOMOSALAT EOCTISALATE:Nausea:0.85), (AZELNIDIPINEOLMESARTAN MEDOXOMIL:Nausea:0.06), (AZITHROMYCIN MONOHYDRATE:Nausea:1.38), (AZITHROMYCIN OR METHOTREXATE SODIUM:Nausea:1.98), (BACILLUS CALMETTE-GUERIN SUBSTRAIN CONNAUGHT LIVE ANTIGEN:Nausea:0.01), (BACITRACIN ZINC:Nausea:0.04), (BACITRACINNEOMYCIN SULFATEPOLYMYXIN B SULFATE:Nausea:0.32), (BAMBUTEROL HYDROCHLORIDE:Nausea:0.03), (BAMIFYLLINE HYDROCHLORIDE:Nausea:1.18), (BAZEDOXIFENE:Nausea:0.36), (BECLOMETHASONE DIPROPIONATEFORMOTEROL:Nausea:1.51), (BEE POLLEN:Nausea:0.49), (BENFOTIAMINE:Nausea:0.02), (BENZOCAINERESORCINOL:Naus ea:0.31), (BENZOIC ACIDHYOSCYAMINE SULFATEMETHENAMINEMETHYLENE BLUEPHENYL SALICYLATE:Nausea:0.23), (BEPOTASTINEBEPOTASTINE BESILATE:Nausea:0.14), (BEPRIDIL HYDROCHLORIDE:Nausea:0.1), (BERAPROST:Nausea:1.44), (BERBERINEGERANIUM:Na usea:0.37), (BETA GLUCAN:Nausea:0.6), (BETAMETHASONE DIPROPIONATECALCIPOTRIENE:Nausea:1.94), (BETAMETHASONE DIPROPIONATECALCIPOTRIENE HYDRATE:Nausea:1.36), (BETAMETHASONE DIPROPIONATEGENTAMICIN SULFATE:Nausea:0.43), (BETAMETHASONEDEXCHLORPHENIRAMINE MALEATE:Nausea:0.28), (BETAMIPRONPANIPENEM:Nausea:0.84), (BEXAROTENE:Naus ea:0.76), (BIFONAZOLE:Nausea:0.04), (BILBERRY:Nausea:0.09), (BLONANSERIN:Nausea:0.4), (BORIC ACID:Nausea:0.59), (BORON:Nausea:0.36), (BOTULISM ANTITOXIN:Nausea:0.33), (BRINZOLAMIDETIMOLOL MALEATE:Nausea:0.75), (BRIVARACETAM:Nausea:0.12), (BUPROPION HYDROBROMIDE:Nausea:0.33), (CAJUPUT OILEUCALYPTUS OILPEPPERMINT OIL:Nausea:0.79), (CALCIUM BICARBONATE:Nausea:1.21), (CALCIUM CARBONATEETIDRONATE DISODIUM:Nausea:1.08), (CALCIUM CITRATECHOLECALCIFEROL:Nausea:0.08), (CALCIUM PANTOTHENATECYANOCOBALAMINNIACINAMIDEPYRIDOXINE HYDROCHLORIDE\RIBOFLAVINTHIAMINE MONONITRATE:Nausea:1.61), (CALCIUM PHOSPHATE:Nausea:0.88), (CALCIUM PHOSPHATE, DIBASIC, ANHYDROUSFERROUS SUCCINATEMAGNESIUM HYDROXIDE:Nausea:0.15), (CAMOSTAT MESYLATE:Nausea:0.67), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:Nausea:0.31), (CAMPHORMENTHOLMETHYL SALICYLATE:Nausea:0.58), (CAPECITABINEOXALIPLATIN:Nausea:1.19), (CAPSICUM:N ausea:0.09), (CARBIDOPAMELEVODOPA:Nausea:0.17), (CARBON DIOXIDE:Nausea:0.33), (CARBOPLATINPACLITAXEL:Nausea:0.26), (CARBOPROST TROMETHAMINE:Nausea:0.09), (CARTEOLOL:Nausea:0.26), (CARTEOLOL HYDROCHLORIDE:Nausea:0.25), (CEFMETAZOLE:Nausea:0.2), (CEFOXITINCEFOXITIN SODIUM:Nausea:0.38), (CEFPIROME SULFATE:Nausea:0.58), (CELIPROLOL:Nausea:0.89), (CELLULASEDIASTASELIPASE:Na usea:0.42), (CEPHARANTHINE:Nausea:0.78), (CEPHRADINE:Nausea:0.35), (CERIVASTATI N SODIUM:Nausea:1.03), (CERTOLIZUMAB:Nausea:0.57), (CHIMAPHILA UMBELLATAMANGANESE CHLORIDEPOPULUS TREMULOIDESSODIUM TAUROCHOLATE:Nausea:0.33), (CHLOROQUINE PHOSPHATE:Nausea:0.15), (CHLORPHENIRAMINE MALEATEHYDROCODONE BITARTRATEPSEUDOEPHEDRINE HYDROCHLORIDE:Nausea:0.06), (CHLORPROPAMIDE:Nausea:0.05), (CHOLECALCIFER OLGENISTEINZINC GLYCINATE:Nausea:0.4), (CHOLINE ALFOSCERATE:Nausea:0.19), (CHOLINETRAZODONE HYDROCHLORIDE:Nausea:0.42), (CICLOPIROXCICLOPIROX OLAMINE:Nausea:0.52), (CIFENLINE:Nausea:0.24), (CIFENLINE SUCCINATE:Nausea:0.34), (CITICOLINE:Nausea:0.14), (CITRIC ACID MONOHYDRATE\POTASSIUM CITRATE:Nausea:0.29), (CITRIC ACID MONOHYDRATETRISODIUM CITRATE DIHYDRATE:Nausea:0.32), (CLENBUTEROL HYDROCHLORIDE:Nausea:0.42), (CLOCORTOLONE PIVALATE:Nausea:0.99), (CLODRONATE DISODIUM:Nausea:1.62), (CLODRONIC ACID:Nausea:0.41), (CLOPERASTINE:Nausea:0.35), (CLOSTRIDIUM TETANI TOXOID ANTIGEN (FORMALDEHYDE INACTIVATED):Nausea:1.04), (TETANICLOSTRIDIUM TETANI TOXOID ANTIGEN (FORMALDEHYDE INACTIVATED)DIPHTHERIAE TOXOID ANTIGEN (FORMALDEHYDE INACTIVATED):Nausea:0.69), (CLOXACILLIN SODIUM:Nausea:1.17), (COBALAMIN:Nausea:0.72), (CODEINE PHOSPHATEDICLOFENAC SODIUM:Nausea:0.56), (CODEINE PHOSPHATEGUAIFENESINPSEUDOEPHEDRINE HYDROCHLORIDE:Nausea:0.3), (COLCHICINEDICYCLOMINE HYDROCHLORIDE:Nausea:0.93), (COLISTIN SULFATE:Nausea:0.02), (COLLAGEN:Nausea:1.2), (COUMARINTROXERUTIN:Nausea:0.15), (CREATINE MONOHYDRATE:Nausea:0.22), (CREATININE:Nausea:0.12), (CROMOLYN:Nausea:0.14), (C UMIN:Nausea:0.14), (CYANOCOBALAMINDICLOFENAC SODIUMPYRIDOXINE HYDROCHLORIDETHIAMINE MONONITRATE:Nausea:0.36), (CYANOCOBALAMINFOLIC ACIDIRON:Nausea:0.09), (CYANOCOBALAMINPYRIDOXINE HYDROCHLORIDETHIAMINE DISULFIDE:Nausea:1.37), (CYCLOPHOSPHAMIDEDOXORUBICINPREDNISONERITUX IMABVINCRISTINE:Nausea:0.41), (CYCLOPHOSPHAMIDEEPIRUBICIN HYDROCHLORIDEFLUOROURACIL:Nausea:0.1), (CYSTEINE:Nausea:0.5), (DARUNAVIR RITONAVIR:Nausea:0.17), (DAUNORUBICIN CITRATE:Nausea:0.05), (DEMECLOCYCLINE:Nausea:0.56), (DEMECLOCYCLINE HYDROCHLORIDE:Nausea:0.49), (DEOXYRIBONUCLIEC ACID:Nausea:0.41), (DIACEREIN:Nausea:0.24), (DIBUCAINE:Nausea:0.08), (DICHLORPHEN AMIDE:Nausea:0.11), (DICLOFENAC DIETHYLAMINE:Nausea:0.53), (DIETARY SUPPLEMENTFLAVOCOXID:Nausea:0.24), (DIGITALIS:Nausea:0.16), (DIMETHYL SULFOXIDE:Nausea:0.83), (DIPHENHYDRAMINE HYDROCHLORIDEDOXYLAMINE SUCCINATE:Nausea:0.48), (DIPYRONEPROMETHAZINE HYDROCHLORIDE:Nausea:2.46), (DISOPYRAMIDE:Nausea:0.75), (DISTIGMINE BROMIDE:Nausea:0.66), (DOBUTAMINE HYDROCHLORIDE:Nausea:1.84), (DOXIFLURIDINE:Nausea:0.62), (DROSPIRENONEEST RADIOL:Nausea:0.22), (DROSPIRENONEETHINYL ESTRADIOLLEVOMEFOLATE CALCIUM:Nausea:1.11), (DYCLONINE HYDROCHLORIDE:Nausea:0.16), (DYDROGESTERONEESTRADIOL:Nausea:0.68), (ECAB ET:Nausea:0.16), (ECABET SODIUM:Nausea:0.33), (PURPUREA:Nausea:0.11), (EFALIZUMAB:Nausea:0.04), (EFLORNITHINE HYDROCHLORIDE:Nausea:0.29), (ELECTROLYTES NOSPOLYETHYLENE GLYCOL 3350:Nausea:0.38), (EMPAGLIFLOZINLINAGLIPTIN:Nausea:1.95), (EMPAGLIFLOZINME TFORMIN HYDROCHLORIDE:Nausea:0.01), (EMTRICITABINERILPIVIRINE HYDROCHLORIDETENOFOVIR DISOPROXIL FUMARATE:Nausea:0.81), (EMTRICITABINERILPIVIRINETENOFOVIR DISOPROXIL FUMARATE:Nausea:0.57), (EMTRICITABINETENOFOVIR:Nausea:0.4), (EMTRICITABINE TENOFOVIR ALAFENAMIDE FUMARATE:Nausea:0.33), (ENFUVIRTIDE:Nausea:0.13), (EPACADOSTAT:Nausea:0.88), (E PHEDRINE HYDROCHLORIDEGUAIFENESIN:Nausea:0.82), (EPROSARTAN MESYLATE:Nausea:0.17), (EPROSARTAN MESYLATEHYDROCHLOROTHIAZIDE:Nausea:0.26), (ERGOLOID MESYLATES:Nausea:0.51), (ESTRADIOL ACETATE:Nausea:0.91), (ESTRADIOL BENZOATE:Nausea:1.02), (ESTRADIOL HEMIHYDRATE:Nausea:0.63), (ETEPLIRSEN:Nausea:0.11), (ETHINYL ESTRADIOL:Nausea:0.53), (ETHINYL ESTRADIOLETHYNODIOL:Nausea:0.45), (ETHINYL ESTRADIOLFERROUS FUMARATENORETHINDRONE:Nausea:0.71), (ETHINYL ESTRADIOLGESTODENE:Nausea:0.94), (ETHIODIZED OIL:Nausea:0.74), (ETHYL ICOSAPENTATE:Nausea:0.02), (ETHYL ICOSAPENTATEICOSAPENT:Nausea:0.46), (EUCALYPTOLMENTHOLMETHYL SALICYLATETHYMOL:Nausea:0.54), (EVOFOSFAMIDE:Nausea:0.25), (EZOGABINE:Naus ea:0.01), (FECAL MICROBIOTA:Nausea:0.27), (FERRIC OXYHYDROXIDE:Nausea:0.5), (FERRIC POLYSACCHARIDE COMPLEX:Nausea:0.99), (FINGOLIMOD:Nausea:0.66), (FLAVIN ADENINE DINUCLEOTIDE:Nausea:0.95), (FLOMOXEF:Nausea:0.61), (FLOPROPIONE:Nausea:0.03), (F LUCYTOSINE:Nausea:1.9), (FLUPHENAZINE:Nausea:0.79), (FLUPREDNIDENE ACETATEMICONAZOLE NITRATE:Nausea:0.35), (FURSULTIAMINE HYDROCHLORIDE:Nausea:0.53), (FUSIDATE SODIUM:Nausea:0.82), (GADOBENATE DIMEGLUMINE:Nausea:1.29), (GARENOXACIN:Nausea:0.27), (GARLIC OIL:Nausea:0.72), (GELATIN:Nausea:0.48), (GENTIAN VIOLET:Nausea:0.75), (GLUCONATE:Nausea:0.5), (GLYCOPYRROLATEINDACATEROL MALEATE:Nausea:1.75), (GOLD:Nausea:0.07), (GRAPE SEED EXTRACT:Nausea:0.14), (GREEN TEA LEAF:Nausea:0.57), (HALCINONIDE:Nausea:0.5), (HERBALSHOMEOPATHICSGOLDENS EAL:Nausea:0.17), (HERBALSVITIS VINIFERA SEED:Nausea:0.42), (HOMATROPINE HYDROBROMIDE:Nausea:0.52), (HORSE CHESTNUT:Nausea:1.08), (HUMAN COLOSTRUM:Nausea:0.01), (HYDROCHLORIC ACID:Nausea:0.23), (HYDROCORTISONE HEMISUCCINATE:Nausea:0.88), (HYDROCORTISONE PROBUTATE:Nausea:0.22), (HYDROXOCOBALAMIN HYDROCHLORIDE:Nausea:0.01), (HYDROXYPROPYL CELLULOSE:Nausea:0.43), (IBRITUMOMAB TIUXETAN:Nausea:2.45), (IDURSULFASE:Nausea:0.56), (IFENPRODIL:Nausea:0.4), (IGURA TIMOD:Nausea:0.61), (INDIGOTINDISULFONATE SODIUM:Nausea:0.52), (INFLIXIMAB-DYYB:Nausea:2.05), (INTERFERON ALFA-2A OR INTERFERON ALFA-2B:Nausea:1.48), (INTERFERON ALFA-N1:Nausea:0.63), (INTERFERON ALFACON-1:Nausea:0.42), (INTERFERON BETA:Nausea:0.98), (INTERFERON GAMMA:Nausea:0.32), (INTERFERON GAMMA-1B:Nausea:2.99), (IODINE\POTASSIUM IODIDE:Nausea:0.6), (ISOFLAVONES SOY:Nausea:0.37), (ISOFLURANE:Nausea:1.04), (ISONIAZIDPYRIDOXINE:Nausea:0.12), (I SONIAZIDRIFAMPIN:Nausea:1.88), (ISOPROPYL ALCOHOL:Nausea:0.13), (KALLIDINOGENASE:Nausea:0.04), (KONJAC MANNAN:Nausea:0.04), (LACIDIPINE:Nausea:0.85), (LACTITOL:Nausea:0.82), (LACTOBAC ILLUS RHAMNOSUS:Nausea:0.58), (CASEI:Nausea:0.04), (LACTOPEROXIDASE BOVINELYSOZYME HYDROCHLORIDE:Nausea:0.25), (LANOLINMINERAL OILPETROLATUM:Nausea:0.67), (LANOLINPETROLATUM:Nausea:0.24), (LATANOPRO STTIMOLOL:Nausea:1.03), (LETROZOLERIBOCICLIB:Nausea:0.36), (LEVAMLODIPINE: Nausea:0.59), (LEVOBUPIVACAINE:Nausea:0.84), (LEVOCARNITINE HYDROCHLORIDE:Nausea:0.24), (LEVOLEUCOVORIN:Nausea:0.11), (LEVOMEFOLATE CALCIUM\METHYLCOBALAMINPYRIDOXAL 5-PHOSPHATE:Nausea:0.1), (LEVOMEFOLIC ACID:Nausea:0.15), (LEVORPHANOL TARTRATE:Nausea:0.22), (LEVOSIMENDAN:Nausea:0.58), (LINSIDOMINE:Nausea:0.82), (L OPRAZOLAM:Nausea:1.19), (LOVASTATINNIACIN:Nausea:0.21), (LOXAPINE:Nausea:0.17), (LUMACAFTOR:Nausea:0.73), (LUTEINIZING HORMONE:Nausea:0.11), (LUTETIUM LU-177:Nausea:1.37), (LYNESTRENOL:Nausea:0.02), (LYSINE HYDROCHLORIDE:Nausea:0.06), (MAGNESIUM ACETATE:Nausea:0.1), (MAGNESIUM ASPARTATE\POTASSIUM ASPARTATE:Nausea:1.4), (MAGNESIUM GLUCEPTATE:Nausea:0.29), (MANGANESE GLUCONATE:Nausea:0.08), (MANGANESEMANGANESE CHLORIDE:Nausea:0.14), (MANIDIPINE HYDROCHLORIDE:Nausea:0.37), (MAPROTILINE HYDROCHLORIDE:Nausea:1.03), (MARIZOMIB:Nausea:0.36), (MAXACALCITOL:Nausea:00.83), (MEPENZOLATE BROMIDE:Nausea:0.41), (MESTRANOLNORETHINDRONE:Nausea:0.67), (METAPROTER ENOL SULFATE:Nausea:0.05), (METFORMIN HYDROCHLORIDESAXAGLIPTIN:Nausea:1.11), (METHENAMINE:Nausea:1.48), (METHO XSALEN:Nausea:0.02), (METHYLENE BLUE:Nausea:1.17), (METHYLPREDNISOLONE OR METHYLPREDNISOLONE ACETATE:Nausea:1.02), (METYRAPONE:Nausea:0.75), (MEXILETINE:Nausea:1.52), (MIGL USTAT:Nausea:1.73), (MILRINONE LACTATE:Nausea:0.23), (NATURAL LATEX RUBBER:Nausea:0.13), (NEDAPLATIN:Nausea:0.14), (NEFAZODONE:Nausea:1.15), (NEOST IGMINE BROMIDE:Nausea:0.27), (NEOSTIGMINE METHYLSULFATE:Nausea:0.86), (NIACINAMIDEPYRIDOXINE\RIBOFLAVINTHIAMIN E:Nausea:0.26), (NICERGOLINE:Nausea:0.09), (NILUTAMIDE:Nausea:0.26), (NILVADIPINE: Nausea:0.1), (NIMUSTINE:Nausea:0.15), (NIPRADILOL:Nausea:0.17), (NITRIC OXIDE:Nausea:0.19), (NUSINERSEN:Nausea:1.18), (NYSTATINTRIAMCINOLONE ACETONIDE:Nausea:0.62), (OK-432:Nausea:0.43), (OLODATEROL HYDROCHLORIDE:Nausea:1.2), (OMBITASVIR HEMINONAHYDRATEPARITAPREVIR DIHYDRATERITONAVIR:Nausea:0.01), (OMEGA-6 FATTY ACIDS:Nausea:0.7), (OPICAPONE:Nausea:0.33), (OPRELVEKIN:Nausea:0.34), (OXANDROL ONE:Nausea:0.89), (OXATOMIDE:Nausea:0.38), (PAMIDRONIC ACID:Nausea:0.75), (PAPAYA:Nausea:0.69), (PAROXETINE MESYLATE:Nausea:0.39), (PARSLEY:Nausea:0.38), (PASCOPYRUM SMITHII POLLEN:Nausea:0.2), (PAZUFLOXACIN:Nausea:0.01), (PAZUFLOXACIN MESILATE:Nausea:0.74), (PEGINTERFERON ALFA-2A OR PEGINTERFERON ALFA-2B OR PEGINTERFERON BETA-1A OR PEGINTERFERON LAMBDA-1A:Nausea:0.72), (PEGZEREPOETIN ALFA:Nausea:0.66), (PENTETRAZOL:Nausea:0), (PENTOSTATIN:Nausea:3.28), (PERAMPA NEL:Nausea:0.63), (PEROSPIRONE HYDROCHLORIDE:Nausea:0.08), (PHENELZINE:Nausea:0.43), (PHENOBARBITAL SODIUM:Nausea:1), (PHENYLEPHRINE HYDROCHLORIDEWITCH HAZEL:Nausea:1.07), (PIMOBENDAN:Nausea:0.96), (PIRENZEPINE:Nausea:0.33), (POLIDO CANOL:Nausea:0.37), (POLYETHYLENE GLYCOL 3350POTASSIUM CHLORIDE\SODIUM BICARBONATE\SODIUM CHLORIDE\SODIUM SULFATE\SODIUM SULFATE ANHYDROUS:Nausea:0.11), (POLYETHYLENE GLYCOL 400:Nausea:1.07), (POLYMYXIN:Nausea:1.26), (POLYMYXIN B:Nausea:0.48), (POLYMYXIN B SULFATETRIMETHOPRIM SULFATE:Nausea:0.26), (POLYSTYRENE SULFONIC ACID:Nausea:0.4), (POTASSIUM ACETATE:Nausea:0.05), (POTASSIUM BROMIDE:Nausea:0.22), (POTASSIUM CITRATETRISODIUM CITRATE DIHYDRATE:Nausea:1.21), (PRANLUKAST:Nausea:1.6), (PREDNISOLONE ACETATESULFACETAMIDE SODIUM:Nausea:0.51), (PREDNISONE ACETATE:Nausea:0.67), (PREGNENOLONE:Nausea:1.08), (PROBUCOL:Nausea:0.09), (PRO CAINE HYDROCHLORIDE:Nausea:0.26), (PROCYCLIDINE:Nausea:0.72), (PROCYCLIDINE HYDROCHLORIDE:Nausea:0.03), (PROMAZINE:Nausea:0.12), (PROPANTHELINE:Nausea:00.29), (PROPARACAINE:Nausea:0.35), (PRUCALOPRIDE:Nausea:0.63), (PRUCALOPRIDE SUCCINATE:Nausea:1.67), (PSORALEN:Nausea:0.46), (PYGEUM:Nausea:0.47), (PYRIDOXAL PHOSPHATE:Nausea:0.96), (PYRIMETHAMINE:Nausea:1.66), (QUAZEPAM:Nausea:0.22), (R ADIUM:Nausea:0.02), (RANIMUSTINE:Nausea:0.56), (RASAGILINE:Nausea:0.87), (REBOXE TINE MESYLATE:Nausea:0.15), (RED CLOVER:Nausea:0.24), (RESLIZUMAB:Nausea:0.49), (RHUBARB:Nausea:0.57), (RILOTUM UMAB:Nausea:0.76), (RILPIVIRINE:Nausea:0.14), (RILUZOLE:Nausea:0.29), (RIMANTADIN E HYDROCHLORIDE:Nausea:0.65), (ROXATIDINE ACETATE HYDROCHLORIDE:Nausea:0.05), (RUPATADINE FUMARATE:Nausea:1.16), (SAFINAMIDE:Nausea:0.82), (SAFINAMIDE MESYLATE:Nausea:0.56), (SAMARIUM SM-153 LEXIDRONAM PENTASODIUM:Nausea:0.93), (SARPOGRELATE HYDROCHLORIDE:Nausea:0.51), (SEBELIPASE ALFA:Nausea:0.21), (SERTINDOLE:Nausea:0.05), (SHARK CARTILAGESHARK, UNSPECIFIED:Nausea:0.2), (SIBUTRAMINE HYDROCHLORIDE:Nausea:1.79), (SILICON DIOXIDE:Nausea:1.93), (SILTUXIMAB:Nausea:0.14), (SITAFLOXACIN:Nausea:0.48), (SIVEL ESTAT SODIUM:Nausea:0.12), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Nausea:1.11), (SOMATOSTATIN:Nausea:1), (SOMATREM:Nausea:0.34), (SORAFENIB TOSYLATE:Nausea:0.43), (STANNOUS FLUORIDE:Nausea:0), (STAVUDINE:Nausea:0.4), (STREPTOMYCIN\STREPTOMYCIN SULFATE:Nausea:0.94), (STRONTIUM:Nausea:0.08), (STRONTIUM RANELATE:Nausea:0.12), (SULBUTIAMINE:Nausea:0.14), (SULFACETAMIDE SODIUMSULFUR:Nausea:0.9), (SULFAMERAZINETRIMETHOPRIM:Nausea:0.52), (SUPL ATAST TOSILATE:Nausea:1.18), (TABALUMAB:Nausea:0.74), (TAFAMIDIS MEGLUMINE:Nausea:0.63), (TALIGLUCERASE ALFA:Nausea:0.86), (TAZOBACTAM SODIUM:Nausea:0.45), (TECELEUKIN:Nausea:0.29), (TEDIZOLID:Nausea:0.01), (TEGAFUR: Nausea:0.77), (TEGAFURURACIL:Nausea:1.25), (TEGASEROD:Nausea:0.6), (TEMOCAPRIL: Nausea:0.38), (TEMOCAPRIL HYDROCHLORIDE:Nausea:0.4), (TENELIGLIPTIN:Nausea:0.41), (TENIPOSIDE:Nausea:0.31), (TENOXICAM:Nausea:0.32), (THIAMYLAL:Nausea:0.4), (THIAMYLAL SODIUM:Nausea:0.98), (THIOGUANINE ANHYDROUS:Nausea:0.14), (THIOTHIXENE:Nausea:0.71), (THYROTROPIN ALFA:Nausea:0.27), (TIAPRIDE HYDROCHLORIDE:Nausea:0.05), (TIBOLONE:Nausea:1.48), (TIMOLOLTRAVOPROST:Na usea:0.46), (TINIDAZOLE:Nausea:0.81), (TIQUIZIUM BROMIDE:Nausea:0.35), (TOFISOPAM:Nausea:0.34), (TOLMETIN SODIUM:Nausea:0.18), (TOREMIFENE:Nausea:0.56), (TRANYLCYPROMINE SULFATE:Nausea:0.52), (TRICHLOROACETIC ACID:Nausea:0.2), (TRIFLUOPERAZINE HYDROCHLORIDE:Nausea:0.01), (TRIMIPRAMINE MALEATE:Nausea:2.84), (TROSPIUM:Nausea:1.21), (TROXIPIDE:Nausea:0.27), (UBENIMEX: Nausea:0), (URACIL:Nausea:0.78), (VADASTUXIMAB TALIRINE:Nausea:0.44), (VALRUBICIN:Nausea:0.13), (VINDESINE SULFATE:Nausea:0.38), (VINPOCETINE:Nausea:0.63), (VITAMIN A PALMITATE:Nausea:0.11), (WARFARIN POTASSIUM:Nausea:2.01), (YEAST:Nausea:1.87), (YTTRIUM Y-90:Nausea:0.23), (ZINC CITRATE:Nausea:1.01), (ZOTEPINE:Nausea:0.02), (.ALPHA.1-PROTEINASE INHIBITOR HUMAN:Neck pain:0.31), (ACARBOSE:Neck pain:0.66), (ACETAMINOPHENPROPOXYPHENE NAPSYLATE:Neck pain:0.24), (ACITRETIN:Neck pain:0.07), (AGALSIDASE BETA:Neck pain:0.2), (ALBIGLUTIDE:Neck pain:0.5), (ALEMTUZUMAB:Neck pain:0.47), (ALENDRONIC ACID:Neck pain:0.8), (ALFUZOSIN HYDROCHLORIDE:Neck pain:0.16), (ALISKIREN HEMIFUMARATE:Neck pain:0.23), (AMINO ACIDSDEXTROSEELECTROLYTES NOS:Neck pain:0.13), (AMISULPRIDE:Neck pain:0.25), (AMLODIPINE BESYLATEATORVASTATIN CALCIUM:Neck pain:0.09), (AMLODIPINE BESYLATEHYDROCHLOROTHIAZIDEOLMESARTAN MEDOXOMIL:Neck pain:0.54), (AMLODIPINE BESYLATEVALSARTAN:Neck pain:0.18), (AMLODIPINEVALSARTAN:Neck pain:0.2), (AMMONIUM LACTATE:Neck pain:0.21), (ANTIHISTAMINES NOS:Neck pain:0.19), (BENAZEPRILBENAZEPRIL HYDROCHLORIDE:Neck pain:0.92), (BETAMETHASONE DIPROPIONATECALCIPOTRIENECALCIPOTRIENE HYDRATE:Neck pain:0.21), (BRINZOLAMIDE:Neck pain:0.66), (BUDESONIDEFORMOTEROL FUMARATE:Neck pain:0.52), (CABOZANTINIB S-MALATE:Neck pain:0.07), (CANAKINUMAB:Neck pain:0.35), (CANDESARTAN CILEXETILHYDROCHLOROTHIAZIDE:Neck pain:1.62), (CARBOXYMETHYLCELLULOSE SODIUM:Neck pain:0.13), (CARVEDILOL PHOSPHATE:Neck pain:1.41), (CHLORPHENIRAMINEHYDROCODONE:Neck pain:0.21), (CHLORPROMAZINE:Neck pain:0.04), (CHOLESTYRAMINE:Neck pain:1.68), (CLINDAMYCIN:Neck pain:1.2), (CLOSTRIDIUM BUTYRICUM SPORES STRAIN M-55:Neck pain:0.13), (CLOZAPINE:Neck pain:1.11), (COD LIVER OIL:Neck pain:0.61), (CORTICOTROPIN:Neck pain:0.69), (CRIZOTINIB:Neck pain:0.27), (CYPROHEPTADINE:Neck pain:0.05), (DABIGATRAN:Neck pain:0.46), (DANAZOL:Neck pain:0.37), (DASATINIB:Neck pain:1.67), (DEFLAZACORT:Neck pain:0.78), (DIGITOXIN:Neck pain:0.09), (DOLASETRON MESYLATE:Neck pain:0.19), (DOXEPIN HYDROCHLORIDE:Neck pain:0.55), (DUTASTERIDE:Neck pain:1.52), (ECONAZOLE:Neck pain:0.11), (EMPAGLIFLOZINLINAGLIPTIN:Neck pain:0.85), (ETRETINATE:Neck pain:0.05), (EVEROLIMUS:Neck pain:1.71), (FENOFIBRIC ACID:Neck pain:0.31), (FLUTICASONE FUROATESALMETEROL XINAFOATE:Neck pain:0.69), (FONDAPARINUX SODIUM:Neck pain:0.11), (FORMOTEROL:Neck pain:0.05), (FORMOTEROL FUMARATE\MOMETASONE FUROATE:Neck pain:0.48), (GEMCITABINE HYDROCHLORIDE:Neck pain:0.41), (GIMERACILOTERACILTEGAFUR:Neck pain:0.61), (GLICLAZIDE:Neck pain:0.9), (GUANFACINE:Neck pain:0.04), (GUANFACINE HYDROCHLORIDE:Neck pain:0.01), (HERBALS:Neck pain:0.04), (HERBALSTURMERIC:Neck pain:1.21), (HYDROCHLOROTHIAZIDETELMISARTAN:Neck pain:0.62), (HYDROXYUREA:Neck pain:0.35), (ILOPROST:Neck pain:0.54), (IMATINIB MESYLATE:Neck pain:1.07), (IMIPRAMINE:Neck pain:0.3), (IMIPRAMINE HYDROCHLORIDE:Neck pain:1.05), (INDAPAMIDEPERINDOPRIL:Neck pain:1.16), (INFLIXIMAB-DYYB:Neck pain:0.03), (INSULIN GLULISINE:Neck pain:0.27), (INTERFERON ALFA-2A:Neck pain:0.21), (INTERFERON ALFA-2B:Neck pain:0.1), (IXAZOMIB:Neck pain:0.08), (LACTOBACILLUS RHAMNOSUS:Neck pain:0.4), (LAPATINIB DITOSYLATE:Neck pain:0.5), (LEDIPASVIRSOFOSBUVIR:Neck pain:1.26), (LEUCOVORIN CALCIUM:Neck pain:0.77), (LEVOLEUCOVORIN CALCIUM:Neck pain:0.41), (LOMITAPIDE MESYLATE:Neck pain:0.8), (MEPOLIZUMAB:Neck pain:0.33), (METFORMIN HYDROCHLORIDEVILDAGLIPTIN:Neck pain:0.18), (METHENAMINE HIPPURATE:Neck pain:1.17), (MUPIROCINMUPIROCIN CALCIUM:Neck pain:0.82), (NATEGLINIDE:Neck pain:0.33), (NORETHINDRONE:Neck pain:0.8), (PANITUMUMAB:Neck pain:0.42), (PANTOPRAZOLE MAGNESIUM:Neck pain:1.74), (PATIROMER:Neck pain:0.32), (PAZOPANIB HYDROCHLORIDE:Neck pain:1.31), (PEMETREXED DISODIUM:Neck pain:0.52), (PENICILLIN:Neck pain:1.21), (PHENOBARBITAL:Neck pain:0.71), (PHENYTOIN SODIUM:Neck pain:0.17), (PHYTONADIONE:Neck pain:0.36), (PIMECROLIMUS:Neck pain:0.27), (PREDNISOLONE ACETATE:Neck pain:0.69), (PYRIDOXINE:Neck pain:0.37), (QUINAPRIL:Neck pain:0.35), (QUINAPRILQUINAPRIL HYDROCHLORIDE:Neck pain:0.02), (RADIATION THERAPY:Neck pain:0.48), (RAMUCIRUMAB:Neck pain:0.34), (SODIUM FLUORIDE:Neck pain:0.12), (SULFAMETHOXAZOLE:Neck pain:0.75), (SULINDAC:Neck pain:0.05), (TAMOXIFEN CITRATE:Neck pain:0.05), (TERBINAFINE HYDROCHLORIDE:Neck pain:0.68), (TETRACYCLINE:Neck pain:1.27), (TINZAPARIN SODIUM:Neck pain:0.61), (TOBRAMYCIN:Neck pain:0.03), (TRANDOLAPRIL:Neck pain:0.12), (TRASTUZUMAB:Neck pain:0.35), (VALGANCICLOVIR HYDROCHLORIDE:Neck pain:0.67), (VANCOMYCIN HYDROCHLORIDE:Neck pain:0.23), (VINORELBINE TARTRATE:Neck pain:0.21), (PYRAZINAMIDE:Necrotising retinitis:0.56), (ACETAMINOPHENBUTALBITALCAFFEINE:Neoplasm:0.59), (ALBUTERO L SULFATEIPRATROPIUM BROMIDE:Neoplasm:0.79), (ALCOHOL:Neoplasm:0.39), (ALDESLEUKIN:Neoplasm:0.69), (A LENDRONIC ACID:Neoplasm:0.36), (ALFACALCIDOL:Neoplasm:0.09), (ALUMINUM HYDROXIDEDIMETHICONEMAGNESIUM HYDROXIDE:Neoplasm:0.2), (AMISULPRIDE:Neoplasm:0.41), (ARMODAFINIL:Neoplasm:00.24), (BECLOMETHASONE DIPROPIONATE:Neoplasm:0.04), (BENAZEPRILBENAZEPRIL HYDROCHLORIDE:Neoplasm:0.22), (BENDAMUSTINE:Neoplasm:0.23), (BENDAMUSTINE HYDROCHLORIDE:Neoplasm:0.1), (BLINATUMOMAB:Neoplasm:0.04), (BUPRENORPHIN E HYDROCHLORIDE:Neoplasm:0.35), (BUPRENORPHINE HYDROCHLORIDENALOXONE HYDROCHLORIDE:Neoplasm:0.07), (CALCITONIN SALMON:Neoplasm:0.32), (CALCIUM ACETATE:Neoplasm:0.2), (CANDESARTAN CILEXETILHYDROCHLOROTHIAZIDE:Neoplasm:0.95), (CARVEDILOL PHOSPHATE:Neoplasm:0.13), (CERTOLIZUMAB PEGOL:Neoplasm:0.99), (CHLORAMBUCIL:Neoplasm:0.26), (CLOZAPINE:Neoplasm:1.22), (DALTEPARIN SODIUM:Neoplasm:0.89), (DARATUMUMAB:Neoplasm:0.3), (DEXLANSOPRAZOLE:Neoplasm: 1.15), (DOFETILIDE:Neoplasm:0.03), (DOLASETRON MESYLATE:Neoplasm:0.31), (ELETRIPTAN HYDROBROMIDE:Neoplasm:0.35), (ELOTUZUMAB:Neoplasm:0.14), (ENALAPRIL MALEATE:Neoplasm:1.04), (EVOLOCUMAB:Neoplasm:0.18), (FESOTERODINE FUMARATE:Neoplasm:0.18), (FLECAINIDE:Neoplasm:0.53), (FLUDROCORTISONE ACETATE:Neoplasm:0.08), (FLUOCINONIDE:Neoplasm:0.22), (FLUOXETINEFLUOXETIN E HYDROCHLORIDE:Neoplasm:0.6), (FLUTICASONE FUROATESALMETEROL XINAFOATE:Neoplasm:0.11), (FLUTICASONE FUROATEVILANTEROL TRIFENATATE:Neoplasm:0.28), (FUSIDIC ACID:Neoplasm:0.18), (GLYCOPYRRONIUMINDACATEROL:Neoplasm:0.07), (HYDROCH LOROTHIAZIDEIRBESARTAN:Neoplasm:0.59), (HYDROCHLOROTHIAZIDEVALSART AN:Neoplasm:0.4), (ILOPROST:Neoplasm:0.27), (INDAPAMIDEPERINDOPRIL:Neoplasm:0.77), (ISOSORBIDE:Neoplasm:0.31), (RHAMNOSUS:Neoplasm:0.06), (LEDIPASVIRSOFOSBUVIR:Neoplasm:0.55), (LERCANIDI PINE:Neoplasm:1.03), (LEVALBUTEROL HYDROCHLORIDE:Neoplasm:0.44), (LEVOLEUCOVORIN CALCIUM:Neoplasm:0.78), (LISDEXAMFETAMINE DIMESYLATE:Neoplasm:0.05), (MECLIZINEMECLIZINE HYDROCHLORIDE:Neoplasm:0.18), (METFORMIN HYDROCHLORIDEVILDAGLIPTIN:Neoplasm:0), (METHENAMINE HIPPURATE:Neoplasm:0.2), (METHYLPREDNISOLONE ACETATE:Neoplasm:0.53), (MINOCYCLINE HYDROCHLORIDE:Neoplasm:0.24), (MOXIFLOXACIN:Neoplasm:0.35), (NEBIVOLOL:Neo plasm:0.56), (NIFEDIPINE:Neoplasm:0.47), (NIRAPARIB:Neoplasm:0.51), (OMEGA-3 FATTY ACIDS:Neoplasm:0), (OMEGA-3-ACID ETHYL ESTERS:Neoplasm:0.36), (OSIMERTINIB:Neoplasm:0.16), (PAMIDRONATE DISODIUM:Neoplasm:0.61), (PANTOPRAZOLE MAGNESIUM:Neoplasm:0.45), (PENICILLIN:Neoplasm:0.7), (PENTOSTATIN:Neoplasm:0.25), (PHENOBARBITAL:Neoplasm:1.1), (PITAVASTATIN CALCIUM:Neoplasm:0.25), (PONATINIB:Neoplasm:1.22), (PRAMIPEXOLE:Neoplasm:0.21), (PRAVASTATINPRAVASTATIN SODIUM:Neoplasm:0.04), (PRIMIDONE:Neoplasm:0.98), (PROGESTERONE:Neoplasm:0.41), (RABEPRAZOLE:Neoplasm:0.43), (RABEPRAZOLE SODIUM:Neoplasm:0.4), (RAMUCIRUMAB:Neoplasm:0.96), (RANOLAZINE:Neoplasm:0.24), (RISEDRONATE SODIUM:Neoplasm:0.34), (ROFLUMILAST:Neoplasm:0.2), (SACUBITRILVALSARTAN:Ne oplasm:0.47), (SECUKINUMAB:Neoplasm:0.18), (SODIUM OXYBATE:Neoplasm:0.18), (SOTALOL:Neoplasm:0.84), (SUMATRIPTAN:Neoplasm:0.13), (S UMATRIPTANSUMATRIPTAN SUCCINATE:Neoplasm:0.17), (TAMOXIFEN CITRATE:Neoplasm:0.28), (TETRACYCLINE:Neoplasm:0.37), (TIMOLOL:Neoplasm:0.82), (T IPIRACIL HYDROCHLORIDETRIFLURIDINE:Neoplasm:0.25), (TIZANIDINE:Neoplasm:0.24), (VALG ANCICLOVIR HYDROCHLORIDE:Neoplasm:0.73), (.ALPHA.1-PROTEINASE INHIBITOR HUMAN:Neoplasm malignant:0.23), (ACARBOSE:Neoplasm malignant:0.65), (ACTIVATED CHARCOAL:Neoplasm malignant:0.27), (AGALSIDASE BETA:Neoplasm malignant:0.31), (ALCOHOL:Neoplasm malignant:0.88), (ALFACALCIDOL:Neoplasm malignant:0.9), (ALISKIREN HEMIFUMARATE:Neoplasm malignant:0.37), (AMINO ACIDSDEXTROSEELECTROLYTES NOS:Neoplasm malignant:0.55), (AMISULPRIDE:Neoplasm malignant:0.85), (AMLODIPINEVALSARTAN:Neoplasm malignant:0.64), (AMOXICILLINCLARITHROMYCINESOMEPRAZOLE:Neoplasm malignant:0.09), (ASPERGILLUS NIGER .ALPHA.-GALACTOSIDASE:Neoplasm malignant:0.04), (BACITRACIN:Neoplasm malignant:0.54), (BALSALAZIDE DISODIUM:Neoplasm malignant:0.18), (BECLOMETHASONEFORMOTEROL:Neoplasm malignant:0.12), (BENZTROPINE MESYLATE:Neoplasm malignant:0.12), (BETAMETHASONE DIPROPIONATECALCIPOTRIENECALCIPOTRIENE HYDRATE:Neoplasm malignant:0.27), (BETAMETHASONECALCIPOTRIENE:Neoplasm malignant:0.4), (BOCEPREVIR:Neoplasm malignant:0.24), (CALCITONIN:Neoplasm malignant:0.92), (CALCIUM CARBONATEERGOCALCIFEROLRETINOL:Neoplasm malignant:0.3), (CALCIUM\MAGNESIUM\ZINC:Neoplasm malignant:0.17), (CANAGLIFLOZINMETFORMIN HYDROCHLORIDE:Neoplasm malignant:1.22), (CANAKINUMAB:Neoplasm malignant:0.9), (CAPECITABINEOXALIPLATIN:Neoplasm malignant:0.03), (CAPSAICIN:Neoplasm malignant:0.2), (CARBIMAZOLE:Neoplasm malignant:0.44), (CASANTHRANOLDOCUSATE SODIUM:Neoplasm malignant:0.39), (CEFEPIMECEFEPIME HYDROCHLORIDE:Neoplasm malignant:0.18), (CEVIMELINE HYDROCHLORIDE:Neoplasm malignant:0.01), (CICLESONIDE:Neoplasm malignant:0.62), (CILASTATIN SODIUMIMIPENEM:Neoplasm malignant:1.53), (CIPROFIBRATE:Neoplasm malignant:0.06), (CLOMIPRAMINE HYDROCHLORIDE:Neoplasm malignant:0.49), (CORTICOTROPIN:Neoplasm malignant:0.63), (CYCLOPHOSPHAMIDECYTARABINEDEXAMETHASONEDOXORUBI CINMETHOTREXATEVINCRISTINE:Neoplasm malignant:0.34), (DABIGATRAN:Neoplasm malignant:0.47), (DACLIZUMAB:Neoplasm malignant:1.18), (DALTEPARIN:Neoplasm malignant:1.49), (DANAZOL:Neoplasm malignant:1.07), (DASABUVIROMBITASVIRPARITAPREVIRRITONAVIR:Neoplasm malignant:0.19), (DEFIBROTIDE (BOVINE):Neoplasm malignant:0.33), (DEFLAZACORT:Neoplasm malignant:0.89), (DEXAMETHASONETOBRAMYCIN:Neoplasm malignant:0.01), (DEXTROAMPHETAMINE SULFATE:Neoplasm malignant:0.46), (DIGITOXIN:Neoplasm malignant:0.57), (DIPYRIDAMOLE:Neoplasm malignant:0.54), (DOXYLAMINE SUCCINATE:Neoplasm malignant:0.37), (DUTASTERIDETAMSULOSIN HYDROCHLORIDE:Neoplasm malignant:0.02), (ECONAZOLE:Neoplasm malignant:0.56), (EMPAGLIFLOZINLINAGLIPTIN:Neoplasm malignant:0.69), (ESOMEPRAZOLE MAGNESIUMNAPROXEN:Neoplasm malignant:0.21), (ESTROGENS:Neoplasm malignant:0.14), (ESTRONESUS SCROFA OVARY:Neoplasm malignant:0.94), (ETHINYL ESTRADIOLNORGESTIMATE:Neoplasm malignant:0.14), (ETHINYL ESTRADIOLNORGESTREL:Neoplasm malignant:0.07), (ETRETINATE:Neoplasm malignant:0.15), (FERROUS SULFATE ANHYDROUS:Neoplasm malignant:0.43), (FEXOFENADINEPSEUDOEPHEDRINE:Neoplasm malignant:0.52), (FLUDROCORTISONE ACETATE:Neoplasm malignant:1.17), (FLUOROURACILIRINOTECANLEUCOVORIN:Neoplasm malignant:0.36), (FLUTICASONE FUROATE:Neoplasm malignant:0.95), (FORMOTEROL FUMARATE\MOMETASONE FUROATE:Neoplasm malignant:0.29), (GADOBENATE DIMEGLUMINE:Neoplasm malignant:0.25), (GLYCOPYRROLATE:Neoplasm malignant:0.61), (HERBALSSAW PALMETTO:Neoplasm malignant:0.08), (HYDROCHLOROTHIAZIDETELMISARTAN:Neoplasm malignant:0.86), (HYDROCORTISONE ACETATE:Neoplasm malignant:0.32), (HYDROXYCHLOROQUINE DIPHOSPHATE:Neoplasm malignant:0.62), (ILOPROST:Neoplasm malignant:0.27), (IMIPENEM:Neoplasm malignant:0.07), (IMIPRAMINE HYDROCHLORIDE:Neoplasm malignant:1), (IMMUNE GLOBULIN NOS:Neoplasm malignant:0.25), (INDAPAMIDEPERINDOPRIL:Neoplasm malignant:1.16), (INTERFERON:Neoplasm malignant:0.88), (INTERFERON ALFA-2A OR INTERFERON ALFA-2B:Neoplasm malignant:0.49), (INTERFERON GAMMA-1B:Neoplasm malignant:0.31), (LACTASE:Neoplasm malignant:0.84), (LANREOTIDE:Neoplasm malignant:0.18), (LEDIPASVIRSOFOSBUVIR:Neoplasm malignant:2.07), (LEVALBUTEROL:Neoplasm malignant:0.2), (LEVOMEFOLATE CALCIUM\METHYLCOBALAMINPYRIDOXAL PHOSPHATE ANHYDROUS:Neoplasm malignant:0.2), (LOMITAPIDE MESYLATE:Neoplasm malignant:0.69), (MEDROXYPROGESTERONE:Neoplasm malignant:0.08), (MEPOLIZUMAB:Neoplasm malignant:0.05), (METHENOLONE ENANTHATE:Neoplasm malignant:0.13), (METHYLPREDNISOLONE ACETATE:Neoplasm malignant:1.34), (MICONAZOLE NITRATE:Neoplasm malignant:0.27), (MIDODRINE HYDROCHLORIDE:Neoplasm malignant:0.34), (MORPHINE SULFATE\NALTREXONE HYDROCHLORIDE:Neoplasm malignant:0.62), (MOXONIDINE:Neoplasm malignant:0.16), (NALOXONE HYDROCHLORIDETILIDINE HYDROCHLORIDE:Neoplasm malignant:0.44), (NALTREXONE:Neoplasm malignant:0.27), (NATEGLINIDE:Neoplasm malignant:0.53), (NOREPINEPHRINE BITARTRATE:Neoplasm malignant:0.01), (OLODATEROL HYDROCHLORIDETIOTROPIUM BROMIDE MONOHYDRATE:Neoplasm malignant:1.26), (OMEPRAZOLE SODIUM:Neoplasm malignant:0.67), (ORPHENADRINE:Neoplasm malignant:0.02), (OSELTAMIVIR:Neoplasm malignant:0.32), (PARATHYROID HORMONE:Neoplasm malignant:0.08), (PARICALCITOL:Neoplasm malignant:0.15), (PASIREOTIDE:Neoplasm malignant:0.42), (PEGINTERFERON BETA-1A:Neoplasm malignant:1.11), (PENICILLIN:Neoplasm malignant:1.73), (PENTOSTATIN:Neoplasm malignant:1.46), (PENTOXIFYLLINE:Neoplasm malignant:0.11), (PHENPROCOUMON:Neoplasm malignant:0.1), (PIMECROLIMUS:Neoplasm malignant:0.2), (PRAZOSIN:Neoplasm malignant:0.63), (PRAZOSIN HYDROCHLORIDE:Neoplasm malignant:0.53), (PREDNISOLONE SODIUM PHOSPHATE:Neoplasm malignant:1.09), (RIVASTIGMINE TARTRATE:Neoplasm malignant:0.64), (RIZATRIPTAN BENZOATE:Neoplasm malignant:1.36), (SACUBITRILVALSARTAN:Neoplasm malignant:0.91), (SAMARIUM SM-153 LEXIDRONAM PENTASODIUM:Neoplasm malignant:0), (SEVELAMER HYDROCHLORIDE:Neoplasm malignant:0.15), (SIPULEUCEL-T:Neoplasm malignant:0.62), (SOFOSBUVIRVELPATASVIR:Neoplasm malignant:0.16), (TAZOBACTAM:Neoplasm malignant:0.27), (TEDUGLUTIDE\WATER:Neoplasm malignant:0.54), (TERBINAFINE HYDROCHLORIDE:Neoplasm malignant:1.11), (TESTOSTERONE CYPIONATE:Neoplasm malignant:0.08), (TETRACYCLINE:Neoplasm malignant:1.66), (TIGECYCLINE:Neoplasm malignant:0.17), (TIOTROPIUM:Neoplasm malignant:1.04), (UMECLIDINIUM BROMIDEVILANTEROL TRIFENATATE:Neoplasm malignant:1.12), (VARDENAFIL HYDROCHLORIDE TRIHYDRATE:Neoplasm malignant:0.02), (YEAST:Neoplasm malignant:0.01), (ZAFIRLUKAST:Neoplasm malignant:0.44), (ACARBOSE:Neovascular age-related macular degeneration:0.39), (ACETAMINOPHENBUTALBITALCAFFEINE:Neovascular age-related macular degeneration:0.52), (ACETAMINOPHENOXYCODONEOXYCODONE HYDROCHLORIDE:Neovascular age-related macular degeneration:0.16), (ACETAZOLAMIDE:Neovascular age-related macular degeneration:1.68), (ACLIDINIUM BROMIDE:Neovascular age-related macular degeneration:0), (ALISKIREN HEMIFUMARATE:Neovascular age-related macular degeneration:0.22), (AMLODIPINE BESYLATEHYDROCHLOROTHIAZIDEOLMESARTAN MEDOXOMIL:Neovascular age-related macular degeneration:0.11), (AMLODIPINEVALSARTAN:Neovascular age-related macular degeneration:0.25), (AMPHETAMINE ASPARTATEAMPHETAMINE SULFATEDEXTROAMPHETAMINE SACCHARATEDEXTROAMPHETAMINE SULFATE:Neovascular age-related macular degeneration:0.77), (ARMODAFINIL:Neovascular age-related macular degeneration:0.55), (BENAZEPRILBENAZEPRIL HYDROCHLORIDE:Neovascular age-related macular degeneration:0.31), (BISACODYL OR DOCUSATE SODIUM:Neovascular age-related macular degeneration:0.92), (BROMAZEPAM:Neovascular age-related macular degeneration:0.4), (BUDESONIDEFORMOTEROL FUMARATE:Neovascular age-related macular degeneration:0.53), (CALCITONIN SALMON:Neovascular age-related macular degeneration:0.1), (CALCIUM ACETATE:Neovascular age-related macular degeneration:0.4), (CANDESARTAN CILEXETILHYDROCHLOROTHIAZIDE:Neovascular age-related macular degeneration:1.13), (CARFILZOMIB:Neovascular age-related macular degeneration:0.08), (CARVEDILOL PHOSPHATE:Neovascular age-related macular degeneration:1.04), (CERTOLIZUMAB PEGOL:Neovascular age-related macular degeneration:0.4), (CHLORDIAZEPOXIDE HYDROCHLORIDECLIDINIUM BROMIDE:Neovascular age-related macular degeneration:0.11), (CICLESONIDE:Neovascular age-related macular degeneration:0.36), (CIMETIDINE:Neovascular age-related macular degeneration:0.06), (CYCLOBENZAPRINE:Neovascular age-related macular degeneration:0.51), (DAPAGLIFLOZIN PROPANEDIOL:Neovascular age-related macular degeneration:0.06), (DASATINIB:Neovascular age-related macular degeneration:0.68), (DESLORATADINE:Neovascular age-related macular degeneration:0.49), (DEXAMETHASONE SODIUM PHOSPHATEMOXIFLOXACIN HYDROCHLORIDE:Neovascular age-related macular degeneration:0.14), (DIMENHYDRINATE:Neovascular age-related macular degeneration:0.04), (DOCUSATE CALCIUM:Neovascular age-related macular degeneration:0.25), (DOFETILIDE:Neovascular age-related macular degeneration:0.48), (DUTASTERIDETAMSULOSIN HYDROCHLORIDE:Neovascular age-related macular degeneration:0.01), (EDOXABAN TOSYLATE:Neovascular age-related macular degeneration:0.29), (ELETRIPTAN HYDROBROMIDE:Neovascular age-related macular degeneration:0.49), (ESTROGENS, CONJUGATEDMEDROXYPROGESTERONE ACETATE:Neovascular age-related macular degeneration:0.13), (EVEROLIMUS:Neovascular age-related macular degeneration:0.39), (EVOLOCUMAB:Neovascular age-related macular degeneration:0.87), (FORMOTEROL FUMARATE:Neovascular age-related macular degeneration:0.58), (FORMOTEROL FUMARATE DIHYDRATE\MOMETASONE FUROATE:Neovascular age-related macular degeneration:0.26), (FORMOTEROL FUMARATE\MOMETASONE FUROATE:Neovascular age-related macular degeneration:0.2), (GLYCOPYRRONIUMINDACATEROL:Neovascular age-related macular degeneration:0.58), (HYDROXYCHLOROQUINE DIPHOSPHATE:Neovascular age-related macular degeneration:0), (HYDROXYZINE PAMOATE:Neovascular age-related macular degeneration:0.46), (HYOSCYAMINE:Neovascular age-related macular degeneration:0.32), (ILOPROST:Neovascular age-related macular degeneration:0.24), (IMATINIB MESYLATE:Neovascular age-related macular degeneration:0.16), (INDAPAMIDEPERINDOPRIL:Neovascular age-related macular degeneration:0.78), (LERCANIDIPINE:Neovascular age-related macular degeneration:0.62), (LITHIUM CARBONATE:Neovascular age-related macular degeneration:0.41), (MEPOLIZUMAB:Neovascular age-related macular degeneration:0.11), (METAXALONE:Neovascular age-related macular degeneration:1.06), (METHYLPREDNISOLONE SODIUM SUCCINATE:Neovascular age-related macular degeneration:0.72), (MODAFINIL:Neovascular age-related macular degeneration:0.42), (NABUMETONE:Neovascular age-related macular degeneration:0.08), (NADOLOL:Neovascular age-related macular degeneration:0.17), (NALOXONE HYDROCHLORIDEOXYCODONE HYDROCHLORIDE:Neovascular age-related macular degeneration:0.64), (NATEGLINIDE:Neovascular age-related macular degeneration:0.17), (NEBIVOLOL:Neovascular age-related macular degeneration:0.91), (NILOTINIB:Neovascular age-related macular degeneration:0.3), (NINTEDANIB:Neovascular age-related macular degeneration:0.19), (OLANZAPINE:Neovascular age-related macular degeneration:0.4), (OLODATEROL HYDROCHLORIDETIOTROPIUM BROMIDE MONOHYDRATE:Neovascular age-related macular degeneration:0.43), (OXCARBAZEPINE:Neovascular age-related macular degeneration:0.74), (PANTOPRAZOLE MAGNESIUM:Neovascular age-related macular degeneration:1.07), (PONATINIB:Neovascular age-related macular degeneration:0.23), (PROMETHAZINE HYDROCHLORIDE:Neovascular age-related macular degeneration:0.34), (PROMETHAZINEPROMETHAZINE HYDROCHLORIDE:Neovascular age-related macular degeneration:0.47), (RIZATRIPTAN BENZOATE:Neovascular age-related macular degeneration:0.13), (ROFLUMILAST:Neovascular age-related macular degeneration:0.4), (SACUBITRILVALSARTAN:Neovascular age-related macular degeneration:0.48), (SECUKINUMAB:Neovascular age-related macular degeneration:0.17), (SENNOSIDES:Neovascular age-related macular degeneration:0.16), (SODIUM OXYBATE:Neovascular age-related macular degeneration:0.53), (SUMATRIPTANSUMATRIPTAN SUCCINATE:Neovascular age-related macular degeneration:0.4), (TETRACYCLINE:Neovascular age-related macular degeneration:0.44), (TICAGRELOR:Neovascular age-related macular degeneration:0.13), (TIMOLOLTRAVOPROST:Neovascular age-related macular degeneration:0.58), (.ALPHA.-TOCOPHEROLRETINOL:Nephrogenic anaemia:0.5), (ACETAMINOPHENAPRONALIDECAFFEINEPROPYPHENAZONE:Nephro genic anaemia:0.34), (ACETAMINOPHENBUTALBITALCAFFEINE:Nephrogenic anaemia:0.32), (ACETAMINOPHENOXYCODONEOXYCODONE HYDROCHLORIDE:Nephrogenic anaemia:0.1), (ACETAZOLAMIDE:Nephrogenic anaemia:0.91), (ACLIDINIUM BROMIDE:Nephrogenic anaemia:0.07), (ADENINE:Nephrogenic anaemia:0.63), (AGALSIDASE BETA:Nephrogenic anaemia:0.41), (ALISKIREN HEMIFUMARATE:Nephrogenic anaemia:0.07), (ALUMINUM SILICATE:Nephrogenic anaemia:0.39), (AMILORIDE HYDROCHLORIDEHYDROCHLOROTHIAZIDE:Nephrogenic anaemia:0.06), (AMINO ACIDSCALCIUM CHLORIDEMAGNESIUM CHLORIDE\SODIUM CHLORIDE\SODIUM LACTATE:Nephrogenic anaemia:0), (AMITRIPTYLINE HYDROCHLORIDE:Nephrogenic anaemia:0.1), (AMLODIPINE BESYLATEHYDROCHLOROTHIAZIDEVALSARTAN:Nephrogenic anaemia:0.12), (AMMONIUM GLYCYRRHIZATEGLYCINEMETHIONINE:Nephrogenic anaemia:0.37), (AMOXAPINE:Nephrogenic anaemia:0.45), (AMOXICILLINCLAVULANATE POTASSIUM:Nephrogenic anaemia:0.38), (AMOXICILLINCLAVULANIC ACID:Nephrogenic anaemia:0.03), (AMPICILLIN:Nephrogenic anaemia:0.29), (ANAGLIPTIN:Nephrogenic anaemia:0.97), (ANASTROZOLE:Nephrogenic anaemia:0.95), (ATOVAQUONE:Nephrogenic anaemia:0.27), (BECLOMETHASONE DIPROPIONATE:Nephrogenic anaemia:0.62), (BENAZEPRILBENAZEPRIL HYDROCHLORIDE:Nephrogenic anaemia:0.35), (BENIDIPINE:Nephrogenic anaemia:0.42), (BEPRIDIL:Nephrogenic anaemia:1.79), (BERAPROST:Nephrogenic anaemia:0.06), (BETAMETHASONE BUTYRATE PROPIONATE:Nephrogenic anaemia:0.37), (BRINZOLAMIDE:Nephrogenic anaemia:0.05), (BUCILLAMINE:Nephrogenic anaemia:0.88), (BUDESONIDEFORMOTEROL FUMARATE:Nephrogenic anaemia:0.13), (BUFORMIN:Nephrogenic anaemia:0), (BUPRENORPHINE HYDROCHLORIDENALOXONE HYDROCHLORIDE:Nephrogenic anaemia:0.05), (BUPROPION HYDROCHLORIDE:Nephrogenic anaemia:0.79), (CALCITONIN:Nephrogenic anaemia:0.24), (CALCITONIN SALMON:Nephrogenic anaemia:0.07), (CALCIUM CHLORIDE ANHYDROUSPOTASSIUM CHLORIDE\SODIUM CHLORIDE\SODIUM LACTATE:Nephrogenic anaemia:0.02), (CALCIUM LACTATE:Nephrogenic anaemia:0.68), (CAMPHORMENTHOLMETHYL SALICYLATE:Nephrogenic anaemia:0.37), (CANAGLIFLOZIN:Nephrogenic anaemia:0.11), (CANAKINUMAB:Nephrogenic anaemia:0.46), (CARVEDILOL PHOSPHATE:Nephrogenic anaemia:0.92), (CEFOTAXIME SODIUM:Nephrogenic anaemia:0.05), (CEPHARANTHINE:Nephrogenic anaemia:0.21), (CERTOLIZUMAB PEGOL:Nephrogenic anaemia:0.92), (CHLORPHENIRAMINE MALEATDIHYDROCODEINE PHOSPHATEMETHYLEPHEDRINE HYDROCHLORIDE, DL-:Nephrogenic anaemia:0.09), (CHOLESTYRAMINE:Nephrogenic anaemia:0.89), (CHOLINE BITARTRATECYSTEINEINOSITOLMAMMAL LIVER:Nephrogenic anaemia:0.02), (CIFENLINE:Nephrogenic anaemia:0.74), (CIPROFLOXACIN HYDROCHLORIDE:Nephrogenic anaemia:0.41), (CLINDAMYCIN:Nephrogenic anaemia:0.55), (CLOBAZAM:Nephrogenic anaemia:0.07), (CLOPERASTINE:Nephrogenic anaemia:0.25), (CLOZAPINE:Nephrogenic anaemia:0.75), (COLESTILAN CHLORIDE:Nephrogenic anaemia:0.12), (CORTISONEHYDROCORTISONE:Nephrogenic anaemia:0.36), (CROTAMITON:Nephrogenic anaemia:0.14), (CYANOCOBALAMINLIDOCAINE HYDROCHLORIDEPYRIDOXINE HYDROCHLORIDETHIAMINE HYDROCHLORIDE:Nephrogenic anaemia:0.01), (DABIGATRAN:Nephrogenic anaemia:0.77), (DANAZOL:Nephrogenic anaemia:0.9), (DAPAGLIFLOZIN PROPANEDIOL:Nephrogenic anaemia:0.12), (DASATINIB:Nephrogenic anaemia:0.68), (DEFLAZACORT:Nephrogenic anaemia:0.44), (DEGARELIX:Nephrogenic anaemia:0.4), (DIAZOXIDE:Nephrogenic anaemia:0.21), (DIGITOXIN:Nephrogenic anaemia:0.76), (DIPHENIDOL HYDROCHLORIDE:Nephrogenic anaemia:0.99), (DIQUAFOSOL TETRASODIUM:Nephrogenic anaemia:0.07), (DOFETILIDE:Nephrogenic anaemia:0.09), (DRONABINOL:Nephrogenic anaemia:0.02), (EDARAVONE:Nephrogenic anaemia:0.47), (EDOXABAN TOSYLATE:Nephrogenic anaemia:2.31), (ELBASVIR:Nephrogenic anaemia:0.03), (ELETRIPTAN HYDROBROMIDE:Nephrogenic anaemia:0.03), (EVOLOCUMAB:Nephrogenic anaemia:0.53), (FAROPENEM SODIUM:Nephrogenic anaemia:0.55), (FLAVIN ADENINE DINUCLEOTIDE:Nephrogenic anaemia:0.54), (FLUTICASONE:Nephrogenic anaemia:0.49), (FLUTICASONE FUROATESALMETEROL XINAFOATE:Nephrogenic anaemia:0.26), (FORMOTEROL FUMARATE:Nephrogenic anaemia:0.04), (FUDOSTEINE:Nephrogenic anaemia:0.25), (FURSULTIAMINE:Nephrogenic anaemia:0.88), (GLUTAMINE\SODIUM GUALENATE:Nephrogenic anaemia:0.33), (GLYCOPYRRONIUMINDACATEROL:Nephrogenic anaemia:0.48), (GRAZOPREVIR:Nephrogenic anaemia:0.01), (HYDROCHLOROTHIAZIDEIRBESARTAN:Nephrogenic anaemia:0.74), (HYDROCHLOROTHIAZIDELISINOPRIL:Nephrogenic anaemia:0.58), (HYDROXYZINE PAMOATE:Nephrogenic anaemia:0.78), (IBRUTINIB:Nephrogenic anaemia:0.57), (IDARUCIZUMAB:Nephrogenic anaemia:0.07), (IFENPRODIL TARTRATE:Nephrogenic anaemia:0.1), (IGURATIMOD:Nephrogenic anaemia:0.47), (ILOPROST:Nephrogenic anaemia:0.08), (IMATINIB MESYLATE:Nephrogenic anaemia:1.12), (INDAPAMIDEPERINDOPRIL:Nephrogenic anaemia:0.59), (IPRAGLIFLOZIN L-PROLINE:Nephrogenic anaemia:0.36), (ISOLEUCINELEUCINEVALINE:Nephrogenic anaemia:0.01), (ITRACONAZOLE:Nephrogenic anaemia:0.58), (RHAMNOSUS:Nephrogenic anaemia:0.03), (LANDIOLOL HYDROCHLORIDE:Nephrogenic anaemia:0.31), (LEDIPASVIRSOFOSBUVIR:Nephrogenic anaemia:1.28), (LITHIUM CARBONATE:Nephrogenic anaemia:0.9), (LULICONAZOLE:Nephrogenic anaemia:0.15), (LYSOZYME HYDROCHLORIDE:Nephrogenic anaemia:0.59), (METFORMIN HYDROCHLORIDEVILDAGLIPTIN:Nephrogenic anaemia:0.2), (METHENAMINE HIPPURATE:Nephrogenic anaemia:0.07), (METHYLPREDNISOLONE ACETATE:Nephrogenic anaemia:0.3), (METOCLOPRAMIDE:Nephrogenic anaemia:0.08), (METRONIDAZOLE:Nephrogenic anaemia:0.82), (METRONIDAZOLEMETRONIDAZOLE HYDROCHLORIDE:Nephrogenic anaemia:0.24), (MINOCYCLINEMINOCYCLINE HYDROCHLORIDE:Nephrogenic anaemia:0.65), (MOSAPRIDE CITRATE DIHYDRATE:Nephrogenic anaemia:0.18), (NADOLOL:Nephrogenic anaemia:0.26), (NADROPARIN CALCIUM:Nephrogenic anaemia:0.23), (NALOXONE HYDROCHLORIDEOXYCODONE HYDROCHLORIDE:Nephrogenic anaemia:0.69), (NICARDIPINE:Nephrogenic anaemia:0.71), (NILOTINIB:Nephrogenic anaemia:1.53), (NINTEDANIB:Nephrogenic anaemia:0.02), (OXYBUTYNIN CHLORIDE:Nephrogenic anaemia:0.18), (OXYGEN:Nephrogenic anaemia:0.83), (PANTOPRAZOLE MAGNESIUM:Nephrogenic anaemia:0.43), (PAZOPANIB HYDROCHLORIDE:Nephrogenic anaemia:0.11), (PEGINTERFERON BETA-1A:Nephrogenic anaemia:0.04), (PENICILLIN:Nephrogenic anaemia:0.51), (PHENOBARBITAL:Nephrogenic anaemia:0.65), (PONATINIB:Nephrogenic anaemia:0.07), (PRAMIPEXOLE DIHYDROCHLORIDE:Nephrogenic anaemia:0.6), (PREDNISOLONE SODIUM SUCCINATE:Nephrogenic anaemia:0.46), (PRIMIDONE:Nephrogenic anaemia:0.84), (PYRIDOSTIGMINE BROMIDE:Nephrogenic anaemia:0.22), (PYRIDOXAL PHOSPHATE:Nephrogenic anaemia:0.03), (RIVASTIGMINE TARTRATE:Nephrogenic anaemia:0.57), (ROFLUMILAST:Nephrogenic anaemia:0.42), (SECUKINUMAB:Nephrogenic anaemia:0.19), (SODIUM GUALENATE MONOHYDRATE:Nephrogenic anaemia:0.17), (SUPLATAST TOSILATE:Nephrogenic anaemia:0.23), (THALIDOMIDE:Nephrogenic anaemia:0.27), (TICLOPIDINE:Nephrogenic anaemia:0.12), (TIOTROPIUM:Nephrogenic anaemia:0.47), (TOPIRAMATE:Nephrogenic anaemia:0.99), (TROSPIUM CHLORIDE:Nephrogenic anaemia:0.03), (UROKINASE:Nephrogenic anaemia:0.11), (ZALTOPROFEN:Nephrogenic anaemia:0.12), (ACARBOSE:Nephrolithiasis:0.26), (ACETAMINOPHENOXYCODONEOXY CODONE HYDROCHLORIDE:Nephrolithiasis:0.06), (AMIODARONE:Nephrolithiasis:0.87), (BISACOD YL:Nephrolithiasis:0.27), (BISACODYL OR DOCUSATE SODIUM:Nephrolithiasis:0.54), (BISOPROLOL FUMARATE:Nephrolithiasis:0.63), (BORTEZOMIB:Nephrolithiasis:0.68), (BUPRENORPHINE HYDROCHLORIDE:Nephrolithiasis:0.25), (CANAGLIFLOZINMETFORMIN HYDROCHLORIDE:Nephrolithiasis:0.29), (CANDESARTAN:Nephrolithiasis:0.56), (CANDES ARTAN CILEXETILHYDROCHLOROTHIAZIDE:Nephrolithiasis:0.31), (CARVEDILOL PHOSPHATE:Nephrolithiasis:0.21), (CLOZAPINE:Nephrolithiasis:0.35), (CODEINE:Nephrolit hiasis:0.29), (DACLIZUMAB:Nephrolithiasis:0.37), (DALFAMPRIDINE:Nephrolithiasis:0.93), (DASATINIB:Nephrolithiasis:0.47), (DESLORATADINE:Nephrolithiasis:0.03), (DIMENHYDRI NATE:Nephrolithiasis:0.2), (DIVALPROEX SODIUM:Nephrolithiasis:1.09), (DOCUSATE:Nephrolithiasis:0.09), (DOXEPIN HYDROCHLORIDE:Nephrolithiasis:0.05), (ELETRIPTAN HYDROBROMIDE:Nephrolithiasis:0.62), (EMPAGLIFLOZINLINAGLIPTIN:Nephrolithiasis: 0.45), (ENOXAPARIN:Nephrolithiasis:0.46), (ESOMEPRAZOLE:Nephrolithiasis:0.33), (FISH OIL\OMEGA-3 FATTY ACIDS:Nephrolithiasis:0.13), (FLUOXETINEFLUOXETINE HYDROCHLORIDE:Nephrolithiasis:0.33), (FLUTICASONE FUROATEVILANTEROL TRIFENATATE:Nephrolithiasis:0.21), (HEPARIN SODIUM:Nephrolithiasis:0.27), (HERBALSTURMERIC:Nephrolithiasis:0.09), (HYDROXYU REA:Nephrolithiasis:0.07), (HYDROXYZINE PAMOATE:Nephrolithiasis:0.84), (ILOPROST:Nephrolithiasis:0.42), (INDAPAMIDE:Nephrolit hiasis:0.11), (INFLUENZA VIRUS VACCINE:Nephrolithiasis:0.09), (INSULIN GLULISINE:Nephrolithiasis:0.1), (LEDIPASVIRSOFOSBUVIR:Nephrolithiasis:0.47), (LENA LIDOMIDE:Nephrolithiasis:1.16), (LERCANIDIPINE:Nephrolithiasis:0.31), (LERCANIDIPINE HYDROCHLORIDE:Nephrolithiasis:0.15), (LIDOCAINE:Nephrolithiasis:0.85), (LIDOCAINE PRILOCAINE:Nephrolithiasis:0.13), (LOMITAPIDE MESYLATE:Nephrolithiasis:0.04), (MIDODRINE:Nephrolithiasis:0.08), (NILOTINIB:Nephrolit hiasis:0.54), (OXCARBAZEPINE:Nephrolithiasis:0.91), (PANTOPRAZOLE MAGNESIUM:Nephrolithiasis:0.7), (PEGINTERFERON BETA-1A:Nephrolithiasis:0.08), (PERINDOPRIL:Nephrolithiasis:0.1), (POLYETHYLENE GLYCOLS:Nephrolithiasis:0.75), (PONATINIB:Nephrolithiasis:0.22), (PREDNISOLONEPRE DNISOLONE ACETATEPREDNISOLONE SODIUM SUCCINATE:Nephrolithiasis:0.1), (PROGESTERONE:Nephrolithiasis:0.09), (RABEPRAZOLE: Nephrolithiasis:0.24), (RALOXIFENE HYDROCHLORIDE:Nephrolithiasis:0.4), (RISEDRONATE SODIUM:Nephrolithiasis:0.08), (ROFLUMILAST:Nephrolithiasis:0.19), (SCOPOLAMINE:Nep hrolithiasis:0.11), (SECUKINUMAB:Nephrolithiasis:0.16), (SULFAMETHOXAZOLE:Nephrolit hiasis:0.16), (TETRACYCLINE:Nephrolithiasis:0.13), (TOCILIZUMAB:Nephrolithiasis:0.13), (T RIAMTERENE:Nephrolithiasis:0.4), (UMECLIDINIUM BROMIDEVILANTEROL TRIFENATATE:Nephrolithiasis:0.65), (AMOXICILLIN:Nephropathy:0.21), (CANDESARTAN CILEXETILHYDROCHLOROTHIAZIDE:Nephropathy:0.32), (CERTOLIZUMAB PEGOL:Nephropathy:0.44), (CLINDAMYCINCLINDAMYCIN PHOSPHATE:Nephropathy:0.32), (CLOTRIMAZOLE:Nephropathy:0.14), (DASATINIB:Nephr opathy:0.17), (DICLOFENAC:Nephropathy:0.39), (DIPHENHYDRAMINE:Nephropathy:0.33), (DOXAZOSINDOXAZOSIN MESYLATE:Nephropathy:0.41), (ENOXAPARIN:Nephropathy:0.09), (EVOLOCUMAB:Nephr opathy:0.83), (FLECAINIDE:Nephropathy:0.15), (FLUTICASONE:Nephropathy:0.05), (HYDRO CHLOROTHIAZIDELISINOPRIL:Nephropathy:0.09), (HYDROCHLOROTHIAZIDEVALSA RTAN:Nephropathy:0.03), (IBRUTINIB:Nephropathy:1), (ILOPROST:Nephropathy:0.69), (IND APAMIDEPERINDOPRIL:Nephropathy:0.13), (LEDIPASVIRSOFOSBUVIR:Nephropathy:0.29), (POLYETHYLENE GLYCOLS:Nephropathy:0.25), (RABEPRAZOLE SODIUM:Nephropathy:0.08), (SECUKINUMAB:Nephropathy:0.07), (SELEXIPAG:Nephropath y:0.57), (SOTALOL:Nephropathy:0.5), (TELMISARTAN:Nephropathy:0.21), (TETRACYCLIN E:Nephropathy:0.01), (TOFACITINIB CITRATE:Nephropathy:0.31), (VERAPAMIL HYDROCHLORIDE:Nephropathy:0.85), (AMIODARONE:Nephrotic syndrome:0.55), (AMOXICILLIN:Nephrotic syndrome:0.23), (ANASTROZOLE:Nephrotic syndrome:0.14), (CEPHALEXIN:Nephrotic syndrome:0.21), (CERTOLIZUMAB PEGOL:Nephrotic syndrome:0.59), (DEVICE:Nephrotic syndrome:0.3), (DEXLANSOPRAZOLE:Nephrotic syndrome:0.35), (DICLOFENAC:Nephrotic syndrome:0.3), (DICLOFENAC SODIUM:Nephrotic syndrome:0.29), (FLUTICASONE FUROATEVILANTEROL TRIFENATATE:Nephrotic syndrome:0.32), (HYDROCHLOROTHIAZIDELISINOPRIL:Nephrotic syndrome:0.04), (IBRUTINIB:Nephrotic syndrome:0.42), (ILOPROST:Nephrotic syndrome:0.19), (NEBIVOLOL:Nephrotic syndrome:0.59), (SECUKINUMAB:Nephrotic syndrome:0.41), (THALIDOMIDE:Nephrotic syndrome:0.06), (TIOTROPIUM BROMIDE MONOHYDRATE:Nephrotic syndrome:0.66), (VENLAFAXINE HYDROCHLORIDE:Nephrotic syndrome:0.26), (VERAPAMIL HYDROCHLORIDE:Nephrotic syndrome:0.71), (ZOLPIDEMZOLPIDEM TARTRATE:Nephrotic syndrome:0.01), (ATRACURIUM:Nerve block:0.23), (ISOFLURANE:Nerve block:0.32), (NEOSTIGMINE:Nerve block:0.32), (SUCCINYLCHOLINE CHLORIDE:Nerve block:0.03), (VECURONIUM BROMIDE:Nerve block:0.41), (ANASTROZOLE:Nerve compression:0.07), (ARMODAFINIL:Nerve compression:0.55), (BISOPROLOL FUMARATE:Nerve compression:0.06), (BUDESONIDE:Nerve compression:0.37), (CERTOLIZUMAB PEGOL:Nerve compression:0.38), (ELETRIPTAN HYDROBROMIDE:Nerve compression:0.08), (ESZOPICLONE:Nerve compression:0.17), (FINGOLIMOD HYDROCHLORIDE:Nerve compression:0.35), (IBRUTINIB:Nerve compression:0.47), (METAXALONE:Nerve compression:0.66), (METHYLPREDNISOLONE ACETATE:Nerve compression:0.08), (METHYLPREDNISOLONE SODIUM SUCCINATE:Nerve compression:0.21), (METRONIDAZOLE:Nerve compression:0.45), (OLANZAPINE:Nerve compression:0.31), (OXCARBAZEPINE:Nerve compression:0.89), (PANTOPRAZOLE:Nerve compression:0.66), (PRAMIPEXOLE DIHYDROCHLORIDE:Nerve compression:0.15), (PRAVASTATIN SODIUM:Nerve compression:0.16), (RALOXIFENE HYDROCHLORIDE:Nerve compression:0.02), (RAMIPRIL:Nerve compression:0.53), (RANOLAZINE:Nerve compression:0.48), (RIZATRIPTAN BENZOATE:Nerve compression:0), (SACUBITRILVALSARTAN:Nerve compression:0.32), (TERIFLUNOMIDE:Nerve compression:0.68), (VITAMIN B:Nerve compression:0.09), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Nerve injury:0.06), (.ALPHA.1-PROTEINASE INHIBITOR HUMAN:Nerve injury:0.34), (ACETAZOLAMIDE:Nerve injury:0.5), (ALBIGLUTIDE:Nerve injury:0.26), (ALCOHOL:Nerve injury:0.12), (ALISKIREN HEMIFUMARATE:Nerve injury:0.3), (AMIODARONE HYDROCHLORIDE:Nerve injury:0.69), (AMLODIPINE BESYLATEHYDROCHLOROTHIAZIDEOLMESARTAN MEDOXOMIL:Nerve injury:0.5), (AMLODIPINEVALSARTAN:Nerve injury:0.02), (BETAMETHASONE DIPROPIONATECALCIPOTRIENECALCIPOTRIENE HYDRATE:Nerve injury:0.13), (BICALUTAMIDE:Nerve injury:0.25), (BRINZOLAMIDE:Nerve injury:0.18), (BUDESONIDEFORMOTEROL FUMARATE:Nerve injury:0.28), (CALCIPOTRIENE:Nerve injury:0.07), (CALCIUM\MAGNESIUM:Nerve injury:0.18), (CALCIUM\MAGNESIUM\ZINC:Nerve injury:0.41), (CANAGLIFLOZINMETFORMIN HYDROCHLORIDE:Nerve injury:0.76), (CANDESARTAN:Nerve injury:1.04), (CANDESARTAN CILEXETIL:Nerve injury:0.98), (CANDESARTAN CILEXETILHYDROCHLOROTHIAZIDE:Nerve injury:0.62), (CAPTOPRIL:Nerve injury:0.28), (CARVEDILOL PHOSPHATE:Nerve injury:1.16), (CETIRIZINE HYDROCHLORIDEPSEUDOEPHEDRINE HYDROCHLORIDE:Nerve injury:0.43), (CHONDROITIN SULFATE (BOVINE)GLUCOSAMINE HYDROCHLORIDE:Nerve injury:0.44), (CLOTRIMAZOLE:Nerve injury:0.51), (CLOZAPINE:Nerve injury:0.32), (DACLIZUMAB:Nerve injury:0.44), (DASATINIB:Nerve injury:0.79), (DEXTROAMPHETAMINE SULFATE:Nerve injury:0.05), (DIMENHYDRINATE:Nerve injury:0.61), (DOMPERIDONE:Nerve injury:0.08), (EDOXABAN TOSYLATE:Nerve injury:0.32), (EMPAGLIFLOZINLINAGLIPTIN:Nerve injury:0.75), (ESTROGENS:Nerve injury:0.3), (ETHINYL ESTRADIOLNORGESTIMATE:Nerve injury:0.26), (FEXOFENADINEPSEUDOEPHEDRINE:Nerve injury:0.24), (FINGOLIMOD HYDROCHLORIDE:Nerve injury:0.37), (FLUVOXAMINE MALEATE:Nerve injury:0.14), (FORMOTEROL FUMARATE:Nerve injury:0.32), (FORMOTEROL FUMARATE\MOMETASONE FUROATE:Nerve injury:0.28), (GLYCOPYRRONIUMINDACATEROL:Nerve injury:0.02), (GUANFACINE:Nerve injury:0.3), (HEPARIN SODIUM:Nerve injury:0.26), (HYDROXYCHLOROQUINE DIPHOSPHATE:Nerve injury:0.31), (ILOPROST:Nerve injury:0.04), (IMIPRAMINE HYDROCHLORIDE:Nerve injury:0.78), (INDAPAMIDE:Nerve injury:0.39), (LACTASE:Nerve injury:0.29), (LEDIPASVIRSOFOSBUVIR:Nerve injury:0.7), (LIDOCAINEPRILOCAINE:Nerve injury:0.42), (LIOTHYRONINE:Nerve injury:0.14), (LOMITAPIDE MESYLATE:Nerve injury:0.66), (MEGESTROL ACETATE:Nerve injury:0.17), (MEPOLIZUMAB:Nerve injury:0.12), (METHENAMINE HIPPURATE:Nerve injury:0.71), (METHYLPREDNISOLONE ACETATE:Nerve injury:0.8), (METHYLPREDNISOLONE SODIUM SUCCINATE:Nerve injury:0.55), (MUPIROCINMUPIROCIN CALCIUM:Nerve injury:0.02), (NALOXONE HYDROCHLORIDEOXYCODONE HYDROCHLORIDE:Nerve injury:0.46), (NATEGLINIDE:Nerve injury:0.19), (NILOTINIB:Nerve injury:0.52), (NORETHINDRONE:Nerve injury:0.75), (OLODATEROL HYDROCHLORIDETIOTROPIUM BROMIDE MONOHYDRATE:Nerve injury:0.12), (OXYBUTYNINOXYBUTYNIN CHLORIDE:Nerve injury:0.05), (PAZOPANIB HYDROCHLORIDE:Nerve injury:0.25), (PEGINTERFERON BETA-1A:Nerve injury:0.86), (PENICILLIN:Nerve injury:1.09), (PERINDOPRIL:Nerve injury:0.03), (PIMECROLIMUS:Nerve injury:0.18), (PONATINIB:Nerve injury:0.73), (PRAZOSIN HYDROCHLORIDE:Nerve injury:0.14), (PREDNISOLONE ACETATE:Nerve injury:0.06), (PROCHLORPERAZINE MALEATE:Nerve injury:0.25), (PROTRIPTYLINE HYDROCHLORIDE:Nerve injury:0.23), (PYRAZINAMIDE:Nerve injury:0.67), (QUINAPRIL:Nerve injury:0), (RABEPRAZOLE:Nerve injury:0.65), (RALOXIFENE HYDROCHLORIDE:Nerve injury:1.47), (ROFLUMILAST:Nerve injury:0.75), (SACUBITRILVALSARTAN:Nerve injury:0.54), (SALMETEROL XINAFOATE:Nerve injury:0.11), (SCOPOLAMINE:Nerve injury:0.48), (SULFAMETHOXAZOLE:Nerve injury:0.28), (TERBUTALINE SULFATE:Nerve injury:0.4), (TETRACYCLINE:Nerve injury:1.06), (TIOTROPIUM:Nerve injury:0.06), (TOCOPHEROL:Nerve injury:0.28), (UMECLIDINIUM BROMIDEVILANTEROL TRIFENATATE:Nerve injury:1.09), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Nervous system disorder:0.03), (.ALPHA.1-PROTEINASE INHIBITOR HUMAN:Nervous system disorder:0.08), (ACARBOSE:Nervous system disorder:0.57), (AMLODIPINE BESYLATEATORVASTATIN CALCIUM:Nervous system disorder:0.01), (AMOXICILLINCLAVULANATE POTASSIUM:Nervous system disorder:0.5), (ARIPIPRAZOLE LAUROXIL:Nervous system disorder:0.18), (AZILSARTAN KAMEDOXOMIL:Nervous system disorder:0.15), (BECLOMETHASONE DIPROPIONATE:Nervous system disorder:0.66), (BRINZOLAMIDE:Nervous system disorder:0.3), (BUMETANIDE:Nervous system disorder:0.8), (BUPRENORPHINE HYDROCHLORIDE:Nervous system disorder:0.82), (CALCIUM ACETATE:Nervous system disorder:0.53), (CALCIUM\MAGNESIUM:Nervous system disorder:0.37), (CALCIUM\MAGNESIUM\ZINC:Nervous system disorder:0.29), (CANAGLIFLOZINMETFORMIN HYDROCHLORIDE:Nervous system disorder:0.29), (CANDESARTAN:Nervous system disorder:1.44), (CANNABIS SATIVA SUBSP. INDICA TOP:Nervous system disorder:0.15), (CARBIDOPA:Nervous system disorder:0.01), (CARVEDILOL PHOSPHATE:Nervous system disorder:1.04), (CICLESONIDE:Nervous system disorder:0.42), (CLINDAMYCIN:Nervous system disorder:0.35), (CLOBETASOL:Nervous system disorder:0.05), (CORTICOTROPIN:Nervous system disorder:0.31), (DANAZOL:Nervous system disorder:0.01), (DEFLAZACORT:Nervous system disorder:0.21), (DESONIDE:Nervous system disorder:0.09), (DIPHENHYDRAMINE:Nervous system disorder:1.03), (DULAGLUTIDE:Nervous system disorder:0.13), (EDOXABAN TOSYLATE:Nervous system disorder:0.51), (ELETRIPTAN HYDROBROMIDE:Nervous system disorder:1.48), (EMPAGLIFLOZINLINAGLIPTIN:Nervous system disorder:0.82), (ENOXAPARIN:Nervous system disorder:1.51), (EPLERENONE:Nervous system disorder:0.34), (ERYTHROMYCIN:Nervous system disorder:0.2), (ESTROGENS, CONJUGATEDMEDROXYPROGESTERONE ACETATE:Nervous system disorder:0.95), (ESTRONESUS SCROFA OVARY:Nervous system disorder:0.1), (ETORICOXIB:Nervous system disorder:0.44), (EVOLOCUMAB:Nervous system disorder:1.5), (FILGRASTIM:Nervous system disorder:0.1), (FISH OIL\OMEGA-3 FATTY ACIDS:Nervous system disorder:0.45), (FLUDROCORTISONE ACETATE:Nervous system disorder:0.37), (FLUOCINONIDE:Nervous system disorder:0.18), (FLUTICASONE FUROATE:Nervous system disorder:0.52), (FORMOTEROL FUMARATE\MOMETASONE FUROATE:Nervous system disorder:0.07), (FULVESTRANT:Nervous system disorder:0.75), (GARLIC:Nervous system disorder:0.01), (GUANFACINE:Nervous system disorder:0.47), (GUANFACINE HYDROCHLORIDE:Nervous system disorder:0.34), (HERBALSTURMERIC:Nervous system disorder:0.7), (HYDROXYCHLOROQUINE DIPHOSPHATE:Nervous system disorder:0.2), (HYDROXYUREA:Nervous system disorder:0.09), (ILOPROST:Nervous system disorder:0.48), (IMIPRAMINE HYDROCHLORIDE:Nervous system disorder:0.6), (INDAPAMIDEPERINDOPRIL:Nervous system disorder:0.39), (INFLUENZA VIRUS VACCINE:Nervous system disorder:0.6), (LEDIPASVIRSOFOSBUVIR:Nervous system disorder:1.01), (LERCANIDIPINE:Nervous system disorder:1.06), (LERCANIDIPINE HYDROCHLORIDE:Nervous system disorder:0.32), (LIDOCAINE HYDROCHLORIDE:Nervous system disorder:0.64), (LOMITAPIDE MESYLATE:Nervous system disorder:0.25), (MEGESTROL ACETATE:Nervous system disorder:0.13), (METHENAMINE HIPPURATE:Nervous system disorder:0.88), (MIDODRINE HYDROCHLORIDE:Nervous system disorder:0.04), (MOXIFLOXACIN HYDROCHLORIDE:Nervous system disorder:1.34), (MYCOPHENOLATE MOFETILMYCOPHENOLATE MOFETIL HYDROCHLORIDE:Nervous system disorder:0.22), (NATEGLINIDE:Nervous system disorder:0.17), (OLODATEROL HYDROCHLORIDETIOTROPIUM BROMIDE MONOHYDRATE:Nervous system disorder:0.09), (PAMIDRONATE DISODIUM:Nervous system disorder:0.1), (PAZOPANIB HYDROCHLORIDE:Nervous system disorder:0.23), (PEGFILGRASTIM:Nervous system disorder:0.32), (PEGINTERFERON BETA-1A:Nervous system disorder:0.7), (PIROXICAM:Nervous system disorder:0.32), (PONATINIB:Nervous system disorder:0.3), (PRAZOSIN:Nervous system disorder:0.55), (PRAZOSIN HYDROCHLORIDE:Nervous system disorder:0.3), (SACUBITRILVALSARTAN:Nervous system disorder:0.98), (SALMETEROL:Nervous system disorder:0.03), (SECUKINUMAB:Nervous system disorder:0.6), (SELEXIPAG:Nervous system disorder:0.11), (TERBINAFINE HYDROCHLORIDE:Nervous system disorder:0.3), (TESTOSTERONE UNDECANOATE:Nervous system disorder:0.01), (TETRACYCLINE:Nervous system disorder:0.38), (TIMOLOL MALEATE:Nervous system disorder:0.25), (TIOTROPIUM:Nervous system disorder:0.35), (TOCILIZUMAB:Nervous system disorder:0.56), (TOLTERODINE TARTRATE:Nervous system disorder:0.94), (TRIAMCINOLONE:Nervous system disorder:0.24), (UMECLIDINIUM BROMIDEVILANTEROL TRIFENATATE:Nervous system disorder:0.86), (ACARBOSE:Nervousness:0.37), (ACETAMINOPHENOXYCODONEOXYCO DONE HYDROCHLORIDE:Nervousness:0.38), (ACETAZOLAMIDE:Nervousness:0.6), (ALBIGLUTI DE:Nervousness:0.08), (ALCOHOL:Nervousness:0.89), (AMANTADINE:Nervousness:0.51), (A MLODIPINE BESYLATEHYDROCHLOROTHIAZIDEOLMESARTAN MEDOXOMIL:Nervousness:0), (AMLODIPINEVALSARTAN:Nervousness:0.12), (AZELASTI NE:Nervousness:0.02), (BENZONATATE:Nervousness:0.3), (BISACODYL:Nervousness:0.55), (BUPRENORPHINE HYDROCHLORIDE:Nervousness:0.98), (BUTYLSCOPOLAMINE BROMIDE:Nervousness:0.16), (CALCIUM ACETATE:Nervousness:0.65), (CANAGLIFLOZINMETFORMIN HYDROCHLORIDE:Nervousness:0.38), (CICLESONIDE:Nervousness:0.31), (CILOSTAZOL: Nervousness:0.11), (CIMETIDINE:Nervousness:0.4), (CINACALCET HYDROCHLORIDE:Nervousness:0.04), (COLESEVELAM HYDROCHLORIDE:Nervousness:0.42), (DABIGATRAN:Nervousness:0.07), (DACLIZUMAB: Nervousness:0.5), (DAPAGLIFLOZIN PROPANEDIOL:Nervousness:0.82), (DASATINIB:Nervousness:1.06), (DOCETAXEL:Nervous ness:0.17), (DOXAZOSIN MESYLATE:Nervousness:0.69), (DULAGLUTIDE:Nervousness:0.13), (EMPAGLIFLOZINLI NAGLIPTIN:Nervousness:0.76), (ENOXAPARIN:Nervousness:1.05), (ERYTHROMYCIN:Nerv ousness:0.06), (ESTROGENS, CONJUGATEDMEDROXYPROGESTERONE ACETATE:Nervousness:0.44), (ETORICOXIB:Nervousness:0.18), (EVEROLIMUS:Nervousnes s:0.93), (EVOLOCUMAB:Nervousness:1.56), (FISH OIL\OMEGA-3 FATTY ACIDS:Nervousness:0.41), (FLECAINIDE:Nervousness:1.14), (FLUDROCORTISONE ACETATE:Nervousness:0.16), (FLUOCINONIDE:Nervousness:0.05), (FLUTICASONE:Nervou sness:0.61), (FLUTICASONE FUROATE:Nervousness:0.37), (FORMOTEROL FUMARATE DIHYDRATE\MOMETASONE FUROATE:Nervousness:0.61), (ILOPROST:Nervousness:0.22), (IMATINIB MESYLATE:Nervousness:0.72), (INDAPAMIDEPERINDOPRIL:Nervousness:0.21), (INDOM ETHACIN:Nervousness:0.46), (INSULIN DEGLUDEC:Nervousness:0.3), (LEDIPASVIRSOFOSBUVIR:Nervousness:0.59), (LERCANID IPINE:Nervousness:0.72), (LIOTHYRONINE SODIUM:Nervousness:0.29), (LISDEXAMFETAMINE DIMESYLATE:Nervousness:1.53), (MEGESTROL ACETATE:Nervousness:0.13), (METHENAMINE HIPPURATE:Nervousness:0.51), (METHYLPREDNISOLONE ACETATE:Nervousness:0.67), (METHYLPREDNISOLONE SODIUM SUCCINATE:Nervousness:0.66), (MIDODRINE:Nervousness:0.32), (NADOLOL:Nervousness: 0.32), (NALOXONE HYDROCHLORIDEOXYCODONE HYDROCHLORIDE:Nervousness:0.58), (NINTEDANIB:Nervousness:0.03), (PANTOPRAZOL E MAGNESIUM:Nervousness:1.17), (PAZOPANIB HYDROCHLORIDE:Nervousness:0.11), (PEGFILGRASTIM:Nervousness:0.09), (PEGINTERF ERON BETA-1A:Nervousness:0.77), (PENICILLIN:Nervousness:0.43), (PERINDOPRIL:Nervousness:0.41), (P ITAVASTATIN CALCIUM:Nervousness:0.11), (PRAZOSIN HYDROCHLORIDE:Nervousness:0.06), (PYRIDOXINE:Nervousness:0.27), (RABEPRAZOLE: Nervousness:0.75), (RIZATRIPTAN BENZOATE:Nervousness:0.74), (SACUBITRILVALSARTAN:Nervousness:1.18), (SALMETE ROL XINAFOATE:Nervousness:0.14), (SENNOSIDES:Nervousness:0.31), (SULFAMETHOXAZOL E:Nervousness:0.11), (TERBINAFINE HYDROCHLORIDE:Nervousness:0.04), (TERBUTALINE SULFATE:Nervousness:0.35), (TETRACYCLINE:Nervousness:0.52), (THALIDOMIDE:Nervou sness:0.4), (TIOTROPIUM:Nervousness:0.36), (TRIAMTERENE:Nervousness:0.8), (VITAMIN A:Nervousness:0.21), (.ALPHA.1-PROTEINASE INHIBITOR HUMAN:Neuralgia:1.42), (ACAMPROSATE:Neuralgia:0.11), (ACECLOFENAC:Neuralgia:0.57), (ALDESLEUKIN:Neuralgia:0.49), (ALISKIREN HEMIFUMARATEHYDROCHLOROTHIAZIDE:Neuralgia:0.14), (ALOGLIPTIN:Neuralgia:00.12), (ALUMINUM HYDROXIDEMAGNESIUM OXIDEOXETHAZAINE:Neuralgia:0.01), (AMBROXOL HYDROCHLORIDE:Neuralgia:0.63), (AMINOPHYLLINE:Neuralgia:0.22), (AMINOSALICYLIC ACID:Neuralgia:0.77), (AMOXICILLINCLARITHROMYCINESOMEPRAZOLE:Neuralgia:10.11), (AMPICILLIN SODIUMSULBACTAM SODIUM:Neuralgia:0.83), (ANAGLIPTIN:Neuralgia:0.18), (ANHYDROUS CITRIC ACIDASPIRIN\SODIUM BICARBONATE:Neuralgia:0.34), (ANTIHISTAMINES NOS:Neuralgia:1.3), (ASCORBIC ACID/CHROMIUM/SELENIUM/VANADYL SULFATE:Neuralgia:0.26), (ASCORBIC ACIDBIOTINCYANOCOBALAMINFOLIC ACIDNIACINPANTOTHENIC ACIDPYRIDOXINE\RIBOFLAVINTHIAMINE:Neuralgia:0.08), (ASPIRINCLOPIDOGREL: Neuralgia:0.56), (ASPIRINLANSOPRAZOLE:Neuralgia:0.23), (ATORVASTATIN CALCIUMEZETIMIBE:Neuralgia:0.24), (AVOCADO OILSOYBEAN OIL:Neuralgia:0.15), (BALSALAZIDE:Neuralgia:0.26), (BAMIFYLLINE HYDROCHLORIDE:Neuralgia:0.39), (BECLOMETHASONE:Neuralgia:1.19), (BENZBROMA RONE:Neuralgia:0.18), (BEPRIDIL:Neuralgia:1.3), (BOSUTINIB:Neuralgia:0.08), (BOTULINU M TOXIN TYPE A:Neuralgia:1.21), (BRINZOLAMIDETIMOLOL MALEATE:Neuralgia:0.44), (BUDESONIDEFORMOTEROL:Neuralgia:0.63), (BUDESONIDE FORMOTEROL FUMARATE:Neuralgia:2.09), (CABOZANTINIB:Neuralgia:0.13), (CABOZANTINIB S-MALATE:Neuralgia:0.62), (CALCIUM CARBONATEERGOCALCIFEROL:Neuralgia:0.46), (CALCIUM CARBONATEETIDRONATE DISODIUM:Neuralgia:0.06), (CALCIUM PANTOTHENATECYANOCOBALAMINNIACINAMIDEPYRIDOXINE HYDROCHLORIDE\RIBOFLAVINTHIAMINE MONONITRATE:Neuralgia:0.58), (CALCIUM\MAGNESIUM:Neuralgia:1.46), (CALCIUMVI TAMINS:Neuralgia:0.27), (CAMPHORDIPHENHYDRAMINE\ZINC OXIDE:Neuralgia:0.15), (CARBAMIDE PEROXIDE:Neuralgia:0.11), (CARBAZOCHROME SODIUM SULFONATE:Neuralgia:0.57), (CARMUSTINE:Neuralgia:0.88), (CARTEOLOL HYDROCHLORIDE:Neuralgia:0.12), (CASPOFUNGIN:Neuralgia:0.28), (CEFDITOREN PIVOXIL:Neuralgia:0.63), (CEFEPIMECEFEPIME HYDROCHLORIDE:Neuralgia:0.49), (CEFOTAXIMECEFOTAXIME SODIUM:Neuralgia:0.34), (CEFPODOXIME PROXETIL:Neuralgia:1.05), (CEFPROZIL:Neuralgia:0.21), (CELIPROLOL:Neuralgia:0.66), (C EPHARANTHINE:Neuralgia:0.05), (CERIVASTATIN SODIUM:Neuralgia:0.37), (CHLORMADINONE ACETATE:Neuralgia:0.16), (CHLOROTHIAZIDE:Neuralgia:0.09), (CHLORPROMAZINE HYDROCHLORIDE:Neuralgia:0.04), (CHLORPROTHIXENE:Neuralgia:0.24), (CHONDROITI N SULFATE (CHICKEN):Neuralgia:0.45), (CICLOPIROX OLAMINE:Neuralgia:0.01), (CIPROFIBRATE:Neuralgia:1.25), (CISAPRIDE:Neuralgia:0.07), (CLADRIBINE:Neuralgia:0.03), (CLENBUTEROL HYDROCHLORIDE:Neuralgia:0.13), (CLIKSTAR:Neuralgia:0.65), (COBICISTATELVITEGR AVIREMTRICITABINETENOFOVIR ALAFENAMIDE FUMARATE:Neuralgia:0.31), (CODEINE PHOSPHATEGUAIFENESIN:Neuralgia:1.3), (COLCHICINEDICYCLOMINE HYDROCHLORIDE:Neuralgia:0.11), (COLISTIN:Neuralgia:0.08), (CRANBERRY JUICE:Neuralgia:0.13), (CYPROTERONE ACETATEETHINYL ESTRADIOL:Neuralgia:0.03), (DABIGATRAN ETEXILATE:Neuralgia:0.12), (DANTROLENE SODIUM:Neuralgia:1.04), (DEFLAZACORT:Neuralgia:2.67), (DESVENLAFAXINE:Neuralgia: 0.62), (DEXPANTHENOLNIACINAMIDEPYRIDOXINE HYDROCHLORIDE\RIBOFLAVIN 5′-PHOSPHATE SODIUMTHIAMINE HYDROCHLORIDE:Neuralgia:0.27), (DILAZEP:Neuralgia:0.52), (DIPHENIDOL HYDROCHLORIDE:Neuralgia:0.43), (DIPYRONEPROMETHAZINE HYDROCHLORIDE:Neuralgia:0.79), (DOBUTAMINE HYDROCHLORIDE:Neuralgia:0.19), (DOLASETRON MESYLATE:Neuralgia:0.93), (DORZOLAMIDE:Neuralgia:1.09), (DOXYLAMINE SUCCINATEPYRIDOXINE HYDROCHLORIDE:Neuralgia:0.32), (ELBASVIRGRAZOPREVIR:Neuralgia:0.76), (ELOTU ZUMAB:Neuralgia:1.2), (EMTRICITABINERILPIVIRINE HYDROCHLORIDETENOFOVIR DISOPROXIL FUMARATE:Neuralgia:0.02), (EMTRICITABINERILPIVIRINETENOFOVIR DISOPROXIL FUMARATE:Neuralgia:0.08), (ENALAPRIL MALEATELERCANIDIPINE HYDROCHLORIDE:Neuralgia:0.61), (ESMOLOL HYDROCHLORIDE:Neuralgia:0.39), (ESTRADIOL BENZOATE:Neuralgia:0.09), (ESTRADIOL HEMIHYDRATE:Neuralgia:0.03), (ETHINYL ESTRADIOLGESTODENE:Neuralgia:0.45), (ETHYL ICOSAPENTATEICOSAPENT:Neuralgia:0.61), (ETOPOSIDE PHOSPHATE:Neuralgia:0.59), (ETRETINATE:Neuralgia:1.93), (EUCALYPTOLMENTHOL METHYL SALICYLATETHYMOL:Neuralgia:0.01), (FERRIC HYDROXIDE:Neuralgia:0.32), (FERROUS BISGLYCINATE:Neuralgia:0.59), (FLUNARIZINE HYDROCHLORIDE:Neuralgia:0.32), (FLUPHENAZINE:Neuralgia:0.31), (FLUTAMIDE:Neura lgia:0.46), (FLUVOXAMINE:Neuralgia:0.56), (FOLIC ACIDIRONMINERALSVITAMINS:Neuralgia:0.24), (FORMOTEROL FUMARATE\MOMETASONE FUROATE:Neuralgia:1.28), (FOSFOMYCIN:Neuralgia:0.56), (FOSPHENYTOINFOSPHENY TOIN SODIUM:Neuralgia:0.5), (FROVATRIPTAN SUCCINATE:Neuralgia:0.35), (FURSULTIAMINE:Neuralgia:0.23), (GALANTAMINE:Neuralg ia:0.06), (GINGER:Neuralgia:0.45), (GLUTAMINE:Neuralgia:0.61), (GLYCOPYRROLATEIN DACATEROL MALEATE:Neuralgia:1.02), (GUANFACINE:Neuralgia:1.69), (HALOPERIDOL DECANOATE:Neuralgia:0.41), (HYDROCHLOROTHIAZIDEMETOPROLOL SUCCINATE:Neuralgia:0.43), (HYDROCHLOROTHIAZIDEQUINAPRIL HYDROCHLORIDE:Neuralgia:0.82), (HYOSCYAMINE SULFATEMETHENAMINEMETHYLENE BLUEPHENYL SALICYLATE\SODIUM PHOSPHATE, MONOBASIC, MONOHYDRATE:Neuralgia:0.48), (IBRITUMOMAB TIUXETAN:Neuralgia:0.72), (ILOPROST:Neuralgia:0.77), (IMIGLUCERASE:Neuralgia:1.3), (I MIPENEM:Neuralgia:0.43), (INSULIN PORKINSULIN PURIFIED PORK:Neuralgia:0.14), (INTERFERON ALFA-2A:Neuralgia:1.72), (INTERFERON ALFA-2B:Neuralgia:1.73), (IRON POLYMALTOSE:Neuralgia:0.23), (IRONPOLYSACCHARIDES:Neuralgia:0.28), (ISONIAZI DPYRAZINAMIDERIFAMPIN:Neuralgia:0.56), (ISOPROPYL ALCOHOLLEUPROLIDE ACETATENORETHINDRONE ACETATE:Neuralgia:0.06), (ITRACONAZOLE\SODIUM CHLORIDE:Neuralgia:0.12), (KETOTIFEN:Neuralgia:0.02), (LACIDIPINE:Neuralgia:0.2), (LA NREOTIDE:Neuralgia:0.34), (LANREOTIDE ACETATE:Neuralgia:0.63), (LAPINE T-LYMPHOCYTE IMMUNE GLOBULIN:Neuralgia:0.25), (LEVALBUTEROL:Neuralgia:0.93), (LEVOCABASTINE HYDROCHLORIDE:Neuralgia:0.35), (LEVOCARNITINE HYDROCHLORIDE:Neuralgia:0.17), (LOMITAPIDE MESYLATE:Neuralgia:1.39), (LUTETIUM LU-177:Neuralgia:0.52), (LYSOZYME HYDROCHLORIDE:Neuralgia:0.11), (MAGNESIUM ASPARTATE\POTASSIUM ASPARTATE:Neuralgia:0.42), (MAGNESIUM GLUCONATE:Neuralgia:0.94), (MANIDIPINE HYDROCHLORIDE:Neuralgia:0.07), (MAXACALCITOL:Neuralgia:0.79), (MENTHOLZINC OXIDE:Neuralgia:0.3), (MESNA:Neuralgia:0.67), (METFORMIN HYDROCHLORIDEROSIGLITAZONE MALEATE:Neuralgia:0.08), (METFORMIN HYDROCHLORIDESAXAGLIPTIN:Neuralgia:0.44), (METFORMINSITAGLIPTIN:Neuralgi a:0.47), (METHENAMINE MANDELATE:Neuralgia:0.41), (METHENOLONE ENANTHATE:Neuralgia:1.09), (METRELEPTIN:Neuralgia:0.7), (MIGLUSTAT:Neuralgia:0.71), (MOEXIPRIL HYDROCHLORIDE:Neuralgia:1.23), (NALBUPHINE HYDROCHLORIDE:Neuralgia:0.21), (NEFAZODONE HYDROCHLORIDE:Neuralgia:0.09), (NEOSTIGMINE:Neuralgia:0.59), (NIMESULIDE:Neural gia:0.15), (NOREPINEPHRINE:Neuralgia:0.42), (NOREPINEPHRINE BITARTRATE:Neuralgia:0.5), (NOREPINEPHRINENOREPINEPHRINE BITARTRATE:Neuralgia:0.05), (OCTREOTIDE:Neuralgia:0.6), (OLANZAPINE PAMOATE:Neuralgia:0.06), (OSELTAMIVIR:Neuralgia:0.62), (OXYBUTYNINOXYBUTYNIN CHLORIDE:Neuralgia:0.92), (PASIREOTIDE:Neuralgia:0.29), (PATIROMER:Neuralgia:1.13), (PEGINTERFERON ALFA-2A OR PEGINTERFERON ALFA-2B:Neuralgia:0.18), (PEGZEREPOETIN ALFA:Neuralgia:0.42), (PENICILLIN V BENZATHINE:Neuralgia:0.21), (PENTAMIDINE ISETHIONATE:Neuralgia:0.87), (PENTOSTATIN:Neuralgia:0.52), (PIMAVANSERIN TARTRATE:Neuralgia:0.19), (PIMOBENDAN:Neuralgia:1.12), (PIZOTYLINE:Neuralgia:0.15), (POTASSIUM IODIDE:Neuralgia:0.38), (PREDNISOLONE SODIUM PHOSPHATE:Neuralgia:1.03), (PREGNENOLONE:Neuralgia:0.14), (PROBUCOL:Neuralgia:0.3), (PROMAZINE HYDROCHLORIDE:Neuralgia:0.11), (PRUCALOPRIDE SUCCINATE:Neuralgia:0.17), (QUINACRINE:Neuralgia:1.09), (RADIUM RA-223 DICHLORIDE:Neuralgia:0.15), (REMIFENTANIL:Neuralgia:0.87), (RETINOL:Neuralgia:1.26), (RILMENIDINE:Neuralgia:0.17), (RILPIVIRINE HYDROCHLORIDE:Neuralgia:0.72), (SARPOGRELATE HYDROCHLORIDE:Neuralgia:0.11), (SEVELAMER HYDROCHLORIDE:Neuralgia:1.72), (SIBUTRAMINE HYDROCHLORIDE:Neuralgia:0.53), (SILICON DIOXIDE:Neuralgia:0.48), (SOFOSBUVIRVELPATASVIR:Neuralgia:1.09), (SUPLATAST TOSILATE:Neuralgia:0.29), (TAMOXIFEN CITRATE:Neuralgia:0.74), (TASIMELTEON:Neuralgia:0.67), (TAURINE:Neuralgia:0.09), (TE DUGLUTIDE:Neuralgia:0.65), (TELOTRISTAT ETHYL:Neuralgia:0.65), (TEMOZOLOMIDE:Neuralgia:0.58), (TEMSIROLIMUS:Neuralgia:0.34), (TENOFOVIR:Neuralgia:0.02), (TESAMORELIN:Neuralgia:0.03), (TETRACYCLINE:Neura lgia:2.49), (THIOPENTAL SODIUM:Neuralgia:0.1), (TILIDINE:Neuralgia:0.31), (TIMOLOL MALEATETRAVOPROST:Neuralgia:0.82), (TIMOLOLTRAVOPROST:Neuralgia:0.14), (TIN ZAPARIN:Neuralgia:1.73), (TOFACITINIB:Neuralgia:0.46), (TRAMETINIB DIMETHYL SULFOXIDE:Neuralgia:0.66), (TRIHEXYPHENIDYL HYDROCHLORIDE:Neuralgia:0.53), (TRIPTORELIN PAMOATE:Neuralgia:0.54), (VECURONIUM BROMIDE:Neuralgia:0.15), (XIPAMIDE:Neuralgia:0.21), (ZIV-AFLIBERCEPT:Neuralgia:0.5), (BUDESONIDE:Neuroendocrine carcinoma:0.06), (BUMETANIDE:Neuroendocrine carcinoma:0.24), (CANDESARTAN:Neuroendocrine carcinoma:0.1), (CELECOXIB:Neuroendocrine carcinoma:0.12), (DILTIAZEM:Neuroendocrine carcinoma:0.47), (EVOLOCUMAB:Neuroendocrine carcinoma:0.04), (FLUTICASONE PROPIONATE:Neuroendocrine carcinoma:0.1), (IBRUTINIB:Neuroendocrine carcinoma:0.65), (INSULIN ASPART:Neuroendocrine carcinoma:0.39), (LEVOLEUCOVORIN CALCIUM:Neuroendocrine carcinoma:0.13), (METHYLPREDNISOLONE SODIUM SUCCINATE:Neuroendocrine carcinoma:0.11), (NEBIVOLOL:Neuroendocrine carcinoma:0.12), (OXYGEN:Neuroendocrine carcinoma:0.05), (PANITUMUMAB:Neuroendocrine carcinoma:0.3), (PRAVASTATIN:Neuroendocrine carcinoma:0.09), (TORSEMIDE:Neuroendocrine carcinoma:0.08), (TRAZODONE HYDROCHLORIDE:Neuroendocrine carcinoma:0.01), (ACARBOSE:Neuroendocrine tumour:0.32), (ADO-TRASTUZUMAB EMTANSINE:Neuroendocrine tumour:0.18), (AMILORIDE HYDROCHLORIDEHYDROCHLOROTHIAZIDE:Neuroendocrine tumour:0.06), (AMLODIPINE BESYLATEHYDROCHLOROTHIAZIDEOLMESARTAN MEDOXOMIL:Neuroendocrine tumour:0.22), (BENAZEPRILBENAZEPRIL HYDROCHLORIDE:Neuroendocrine tumour:0.1), (BICALUTAMIDE:Neuroendocrine tumour:0.53), (BROMAZEPAM:Neuroendocrine tumour:0.75), (CALCIUM ACETATE:Neuroendocrine tumour:0.07), (CARISOPRODOL:Neuroendocrine tumour:0.08), (CARVEDILOL PHOSPHATE:Neuroendocrine tumour:0.32), (CERTOLIZUMAB PEGOL:Neuroendocrine tumour:0.77), (CETUXIMAB:Neuroendocrine tumour:0.45), (CILOSTAZOL:Neuroendocrine tumour:0), (CIMETIDINE:Neuroendocrine tumour:0.4), (CLINDAMYCINCLINDAMYCIN PHOSPHATE:Neuroendocrine tumour:0.45), (CLOZAPINE:Neuroendocrine tumour:0.19), (DABIGATRAN:Neuroendocrine tumour:0.04), (DAPAGLIFLOZIN PROPANEDIOL:Neuroendocrine tumour:0.1), (DASATINIB:Neuroendocrine tumour:0.98), (DESLORATADINE:Neuroendocrine tumour:0.32), (DEVICE:Neuroendocrine tumour:0.56), (DIPHENHYDRAMINE:Neuroendocrine tumour:0.57), (DOCETAXEL:Neuroendocrine tumour:0.09), (DOFETILIDE:Neuroendocrine tumour:0.17), (DOXEPIN HYDROCHLORIDE:Neuroendocrine tumour:0.08), (EDOXABAN TOSYLATE:Neuroendocrine tumour:0.14), (ELETRIPTAN HYDROBROMIDE:Neuroendocrine tumour:0.35), (EMPAGLIFLOZINLINAGLIPTIN:Neuroendocrine tumour:0.13), (ESTROGENS, CONJUGATEDMEDROXYPROGESTERONE ACETATE:Neuroendocrine tumour:0.08), (EVOLOCUMAB:Neuroendocrine tumour:0.71), (FESOTERODINE FUMARATE:Neuroendocrine tumour:0.27), (FLECAINIDE:Neuroendocrine tumour:0.57), (FULVESTRANT:Neuroendocrine tumour:0.09), (FUSIDIC ACID:Neuroendocrine tumour:0.01), (GLYCOPYRRONIUMINDACATEROL:Neuroendocrine tumour:0.05), (HERBALSTURMERIC:Neuroendocrine tumour:0.18), (HYDROXYCHLOROQUINE DIPHOSPHATE:Neuroendocrine tumour:0.01), (HYDROXYZINE PAMOATE:Neuroendocrine tumour:0.38), (IBRUTINIB:Neuroendocrine tumour:0.81), (INDOMETHACIN:Neuroendocrine tumour:0.1), (LEDIPASVIRSOFOSBUVIR:Neuroendocrine tumour:0.29), (LEVALBUTEROL HYDROCHLORIDE:Neuroendocrine tumour:0.05), (LEVOLEUCOVORIN CALCIUM:Neuroendocrine tumour:0.13), (LITHIUM CARBONATE:Neuroendocrine tumour:0.17), (METFORMIN HYDROCHLORIDEVILDAGLIPTIN:Neuroendocrine tumour:0.02), (METHYLPREDNISOLONE ACETATE:Neuroendocrine tumour:0.53), (MOXIFLOXACIN HYDROCHLORIDE:Neuroendocrine tumour:0.21), (NITROFURANTOIN:Neuroendocrine tumour:0.61), (PACLITAXEL:Neuroendocrine tumour:0.71), (PHENOBARBITAL:Neuroendocrine tumour:0.57), (PONATINIB:Neuroendocrine tumour:0.2), (PRIMIDONE:Neuroendocrine tumour:0.6), (PYRIDOXINE:Neuroendocrine tumour:0.2), (RALOXIFENE HYDROCHLORIDE:Neuroendocrine tumour:0.31), (RANOLAZINE:Neuroendocrine tumour:0.84), (REGORAFENIB:Neuroendocrine tumour:0.83), (ROFLUMILAST:Neuroendocrine tumour:0.23), (SACUBITRILVALSARTAN:Neuroendocrine tumour:0.28), (SODIUM OXYBATE:Neuroendocrine tumour:0.34), (SUMATRIPTANSUMATRIPTAN SUCCINATE:Neuroendocrine tumour:0.13), (TERIFLUNOMIDE:Neuroendocrine tumour:0.08), (TETRACYCLINE:Neuroendocrine tumour:0.3), (TIMOLOL:Neuroendocrine tumour:0.43), (TINZAPARIN SODIUM:Neuroendocrine tumour:0.03), (TIOTROPIUM:Neuroendocrine tumour:0.02), (TOCILIZUMAB:Neuroendocrine tumour:0.12), (TOLTERODINE TARTRATE:Neuroendocrine tumour:0.07), (TOPIRAMATE:Neuroendocrine tumour:1.65), (TRIAMCINOLONE ACETONIDE:Neuroendocrine tumour:0.02), (VALACYCLOVIR HYDROCHLORIDE:Neuroendocrine tumour:0.02), (VARENICLINE TARTRATE:Neuroendocrine tumour:0.32), (VEMURAFENIB:Neuroendocrine tumour:0.06), (ZONISAMIDE:Neuroendocrine tumour:0.07), (ACETAMINOPHENCODEINE PHOSPHATE:Neurogenic bladder:0.76), (AMIODARONE:Neurogenic bladder:0.57), (AMIODARONE HYDROCHLORIDE:Neurogenic bladder:0.03), (ANASTROZOLE:Neurogenic bladder:0.1), (BROTIZOLAM:Neurogenic bladder:0.18), (BUMETANIDE:Neurogenic bladder:0.15), (BUPRENORPHINE HYDROCHLORIDENALOXONE HYDROCHLORIDE:Neurogenic bladder:0.26), (BUPROPION:Neurogenic bladder:0.41), (CERTOLIZUMAB PEGOL:Neurogenic bladder:0.23), (CLINDAMYCINCLINDAMYCIN PHOSPHATE:Neurogenic bladder:0.04), (CLOBAZAM:Neurogenic bladder:0.22), (CLONIDINE HYDROCHLORIDE:Neurogenic bladder:0), (COLCHICINE:Neurogenic bladder:0.09), (CORTISONEHYDROCORTISONE:Neurogenic bladder:0.28), (DASATINIB:Neurogenic bladder:0.21), (DICYCLOMINE HYDROCHLORIDE:Neurogenic bladder:0.26), (DILTIAZEM:Neurogenic bladder:0.48), (EPINEPHRINE:Neurogenic bladder:0.19), (ESOMEPRAZOLE:Neurogenic bladder:0.26), (EVOLOCUMAB:Neurogenic bladder:0.68), (FERROUS SULFATE:Neurogenic bladder:0.74), (FLUOXETINEFLUOXETINE HYDROCHLORIDE:Neurogenic bladder:0.36), (GLIPIZIDE:Neurogenic bladder:0.16), (HYDROMORPHONE:Neurogenic bladder:0.09), (INSULIN DETEMIR:Neurogenic bladder:0.14), (INSULIN NOS:Neurogenic bladder:0.54), (ISOSORBIDE:Neurogenic bladder:0.09), (LABETALOLLABETALOL HYDROCHLORIDE:Neurogenic bladder:0.39), (LEDIPASVIRSOFOSBUVIR:Neurogenic bladder:0.16), (METHYLPREDNISOLONE ACETATE:Neurogenic bladder:0.27), (MOXIFLOXACIN HYDROCHLORIDE:Neurogenic bladder:0.2), (NEBIVOLOL:Neurogenic bladder:0), (NITROGLYCERIN:Neurogenic bladder:0.3), (PANTOPRAZOLE:Neurogenic bladder:1.16), (PAROXETINE:Neurogenic bladder:0.25), (PITAVASTATIN CALCIUM:Neurogenic bladder:0.16), (PRAMIPEXOLE:Neurogenic bladder:1), (PRAMIPEXOLE DIHYDROCHLORIDE:Neurogenic bladder:0.81), (PROBIOTICS NOS:Neurogenic bladder:0.17), (PYRIDOXINE HYDROCHLORIDE:Neurogenic bladder:0.06), (RABEPRAZOLE:Neurogenic bladder:0.13), (RANOLAZINE:Neurogenic bladder:0.29), (RIZATRIPTAN BENZOATE:Neurogenic bladder:0.2), (SACUBITRILVALSARTAN:Neurogenic bladder:0.55), (SCOPOLAMINE:Neurogenic bladder:0.08), (SODIUM BICARBONATE:Neurogenic bladder:0.09), (TRIAMTERENE:Neurogenic bladder:0.03), (DEXMEDETOMIDINE HYDROCHLORIDE:Neuromuscular blockade:0.21), (ROPIVACAINE:Neuromuscular blockade:1.42), (BUPIVACAINEEPINEPHRINE:Neuromuscular blockade reversal:0.09), (BUPIVACAINEEPINEPHRINE:Neuromuscular blocking therapy:0.06), (LEVOBUPIVACAINE:Neuromuscular blocking therapy:0.16), (ABOBOTULINUMTOXINA:Neuropathy peripheral:0.28), (ACECLOFENAC:Neuropathy peripheral:0.27), (ACENOCOUMAROL:Neuropathy peripheral:1.36), (ACETAMINOPHEN\CAFFEINEOPIUM:Neuropathy peripheral:0.32), (ACETAMINOPHENCHLORPHENIRAMINE MALEATEPSEUDOEPHEDRINE HYDROCHLORIDE:Neuropathy peripheral:0.46), (ACITRETIN:Neuropathy peripheral:1.29), (ACRIVASTINEPSEUDOEPHEDRINE HYDROCHLORIDE:Neuropathy peripheral:0.09), (ADENINE:Neuropathy peripheral:0.66), (ALBUTEROLIPRATROPIUM BROMIDE:Neuropathy peripheral:0.17), (ALCAFTADINE:Neuropathy peripheral:0.49), (ALDESLEUKIN:Neuropathy peripheral:0.3), (ALISKIREN HEMIFUMARATEHYDROCHLOROTHIAZIDE:Neuropathy peripheral:0.1), (ALUMINUM HYDROXIDEMAGNESIUM HYDROXIDESIMETHICONE:Neuropathy peripheral:0.42), (AMINO ACIDSDEXTROSEELECTROLYTES NOSSOYBEAN OIL:Neuropathy peripheral:0.35), (AMISULPRIDE:Neuropathy peripheral:1.73), (AMLODIPINEPERINDOPRIL:Neuropathy peripheral:0.59), (AMOXICILLINCLARITHROMYCINESOMEPRAZOLE:Neuropathy peripheral:1.03), (AMPICILLIN SODIUM:Neuropathy peripheral:0.21), (AMPICILLIN SODIUMSULBACTAM SODIUM:Neuropathy peripheral:2.26), (ARIPIPRAZOLE LAUROXIL:Neuropathy peripheral:0.56), (ASCORBIC ACID/CHROMIUM/SELENIUM/VANADYL SULFATE:Neuropathy peripheral:0.31), (NIGER ALPHA.-GALACTOSIDASE:Neuropathy peripheral:0.47), (ASPIRIN\CAFFEINESALICYLAMIDE:Neuropathy peripheral:0.22), (ASPIRINCLOPIDOGREL:Neuropathy peripheral:0.2), (ASPIRINMAGNESIUM OXIDE:Neuropathy peripheral:0.9), (ATROPINE SULFATEHYOSCYAMINE SULFATEPHENOBARBITALSCOPOLAMINE HYDROBROMIDE:Neuropathy peripheral:0.18), (AZITHROMYCIN OR METHOTREXATE SODIUM:Neuropathy peripheral:0.05), (BAMIFYLLINE HYDROCHLORIDE:Neuropathy peripheral:0.32), (BECLOMETHASONE:Neuropathy peripheral:1.48), (BECLOMETHASONEFORMOTEROL:Neuropathy peripheral:0.71), (BELATACEPT:Neuropathy peripheral:0.26), (BENIDIPINE:Neuropathy peripheral:0.06), (BENPROPERINE PHOSPHATE:Neuropathy peripheral:0), (BENSERAZIDELEVODOPA:Neuropathy peripheral:0.38), (BENZOCAINE:Neuropathy peripheral:0.28), (BEPRIDIL:Neuropathy peripheral:1.51), (BERAPROST:Neuropathy peripheral:0.72), (BETAMETHASONE DIPROPIONATECALCIPOTRIENE HYDRATE:Neuropathy peripheral:0.31), (BETAMETHASONE SODIUM PHOSPHATENEOMYCIN SULFATE:Neuropathy peripheral:0.21), (BICARBONATE ION:Neuropathy peripheral:0.41), (BILASTINE:Neuropathy peripheral:0.3), (BLONANSERIN:Neuropathy peripheral:0.16), (BOCEPREVIR:Neuropathy peripheral:0.78), (BOSUTINIB:Neuropathy peripheral:0.34), (BOTULINUM TOXIN TYPE A:Neuropathy peripheral:0.59), (BUFORMIN:Neuropathy peripheral:0.01), (BUTYLSCOPOLAMINE:Neuropathy peripheral:0.18), (CABOZANTINIB:Neuropathy peripheral:0.32), (CABOZANTINIB S-MALATE:Neuropathy peripheral:0.82), (CAFFEINEDIPYRONEORPHENADRINE:Neuropathy peripheral:0.14), (CALCIUM CARBONATEETIDRONATE DISODIUM:Neuropathy peripheral:0.05), (CALCIUM CARBONATEMAGNESIUM CARBONATEMAGNESIUM TRISILICATE:Neuropathy peripheral:0.28), (CALCIUM CHLORIDE ANHYDROUSPOTASSIUM CHLORIDE\SODIUM CHLORIDE\SODIUM LACTATE:Neuropathy peripheral:0.42), (CALCIUM CHLORIDEDEXTROSE\POTASSIUM CHLORIDE\SODIUM ACETATE\SODIUM CHLORIDE:Neuropathy peripheral:0.22), (CAMPHORDIPHENHYDRAMINE\ZINC OXIDE:Neuropathy peripheral:0.36), (CARBAMIDE PEROXIDE:Neuropathy peripheral:0.56), (CARBOHYDRATESFATTY ACIDSMINERALSPROTEINVITAMINS:Neuropathy peripheral:0.05), (CARBOMER:Neuropathy peripheral:0.35), (CARBOXYMETHYLCELLULOSE SODIUMGLYCERINPOLYSORBATE 80:Neuropathy peripheral:1.01), (CARBOXYMETHYLCELLULOSE SODIUMHYPROMELLOSES:Neuropathy peripheral:0.04), (CARMUSTINE:Neuropathy peripheral:0.61), (CARTEOLOL HYDROCHLORIDE:Neuropathy peripheral:0.42), (CEFTAZIDIME:Neuropathy peripheral:1.39), (CEFTAZIDIME SODIUM:Neuropathy peripheral:0.1), (CELLULASEDIASTASELIPASE:Neuropathy peripheral:0.53), (CEPHARANTHINE:Neuropathy peripheral:0.75), (CERIVASTATIN SODIUM:Neuropathy peripheral:0.61), (CERTOPARIN SODIUM:Neuropathy peripheral:0.15), (CHLORAMPHENICOL:Neuropathy peripheral:0.79), (CHOLESTEROL:Neuropathy peripheral:0.57), (CICLOPIROX OLAMINE:Neuropathy peripheral:0.29), (CIFENLINE:Neuropathy peripheral:0.5), (CIPROFIBRATE:Neuropathy peripheral:0.56), (CLENBUTEROL HYDROCHLORIDE:Neuropathy peripheral:0.42), (CLOFARABINE:Neuropathy peripheral:0.08), (CLOMIPRAMINE:Neuropathy peripheral:0.15), (CLOMIPRAMINE HYDROCHLORIDE:Neuropathy peripheral:0.88), (CLOPERASTINE:Neuropathy peripheral:0.18), (CODEINE PHOSPHATEPROMETHAZINE:Neuropathy peripheral:0.04), (CODEINE SULFATE:Neuropathy peripheral:0.04), (COLISTIN:Neuropathy peripheral:0.41), (CRANBERRY CONCENTRATE:Neuropathy peripheral:0.1), (DABRAFENIB MESYLATE:Neuropathy peripheral:0.78), (DACLIZUMAB:Neuropathy peripheral:2.26), (DARIFENACIN HYDROBROMIDE:Neuropathy peripheral:0.05), (DAUNORUBICIN:Neuropathy peripheral:0.35), (DESOXIMETASONE:Neuropathy peripheral:0.13), (DIMEMORFAN PHOSPHATE:Neuropathy peripheral:0.28), (DIMETHYL SULFOXIDE:Neuropathy peripheral:0.02), (DIPYRONEPROMETHAZINE HYDROCHLORIDE:Neuropathy peripheral:0.51), (DISOPYRAMIDE:Neuropathy peripheral:0.47), (DOBUTAMINE HYDROCHLORIDE:Neuropathy peripheral:1.05), (DOCUSATESENNOSIDES:Neuropathy peripheral:0.19), (DOLASETRON MESYLATE:Neuropathy peripheral:1.41), (DOPAMINEDOPAMINE HYDROCHLORIDE:Neuropathy peripheral:0.3), (ECONAZOLE:Neuropathy peripheral:1.66), (EDOXABAN TOSILATE MONOHYDRATE:Neuropathy peripheral:0.86), (ELBASVIRGRAZOPREVIR:Neuropathy peripheral:0.22), (EMPAGLIFLOZINMETFORMIN HYDROCHLORIDE:Neuropathy peripheral:0.29), (ENALAPRIL MALEATELERCANIDIPINE HYDROCHLORIDE:Neuropathy peripheral:0.07), (ENSULIZOLEEUCERINOCTINOXATEOCTISALATETITANIUM DIOXIDE\ZINC OXIDE:Neuropathy peripheral:0.29), (EPINASTINE:Neuropathy peripheral:0.13), (EPRAZINONE:Neuropathy peripheral:0.22), (ESTRADIOL ACETATE:Neuropathy peripheral:0.01), (ESTRADIOL BENZOATE:Neuropathy peripheral:0.46), (ESTRADIOLLEVONORGESTREL:Neuropathy peripheral:0.1), (ETHINYL ESTRADIOLNORGESTREL:Neuropathy peripheral:0.81), (EUCALYPTOLMENTHOLMETHYL SALICYLATETHYMOL:Neuropathy peripheral:0.31), (FAROPENEM SODIUM:Neuropathy peripheral:0.79), (FELBAMATE:Neuropathy peripheral:0.4), (FENOTEROL HYDROBROMIDEIPRATROPIUM BROMIDE:Neuropathy peripheral:1.11), (FERRIC CARBOXYMALTOSE:Neuropathy peripheral:0.03), (FERRIC POLYSACCHARIDE COMPLEX:Neuropathy peripheral:0.39), (FERROUS GLYCINE SULFATE:Neuropathy peripheral:0.59), (FERROUS SULFATEFOLIC ACID:Neuropathy peripheral:0.3), (FLUNARIZINE HYDROCHLORIDE:Neuropathy peripheral:0.37), (FLUTAMIDE:Neuropathy peripheral:0.86), (FLUTICASONE PROPIONATEFORMOTEROL FUMARATE:Neuropathy peripheral:0.31), (FORMOTEROL FUMARATE\MOMETASONE FUROATE:Neuropathy peripheral:1.61), (FOSFOMYCIN CALCIUM:Neuropathy peripheral:0.09), (FROVATRIPTAN SUCCINATE:Neuropathy peripheral:0.77), (FURSULTIAMINE:Neuropathy peripheral:0.69), (GADOBENATE DIMEGLUMINE:Neuropathy peripheral:0.18), (GLIMEPIRIDEMETFORMIN:Neuropathy peripheral:0.01), (GLIMEPIRIDEROSIGLITAZONE MALEATE:Neuropathy peripheral:0.17), (GLUCONATE:Neuropathy peripheral:0.13), (GLYCOPYRRONIUMINDACATEROL:Neuropathy peripheral:2.23), (GUANABENZ ACETATE:Neuropathy peripheral:0.72), (HEPARIN CALCIUM:Neuropathy peripheral:0.71), (HERBALSMINERALSVITAMINS:Neuropathy peripheral:0.2), (HIGH DENSITY POLYETHYLENE:Neuropathy peripheral:0.05), (HORSE CHESTNUT:Neuropathy peripheral:0.19), (HYDROCHLOROTHIAZIDERAMIPRIL:Neuropathy peripheral:0.16), (HYDROCORTISONE PROBUTATE:Neuropathy peripheral:0.15), (IDARUBICIN:Neuropathy peripheral:0.65), (IGURATIMOD:Neuropathy peripheral:0.72), (IMIGLUCERASE:Neuropathy peripheral:1.03), (INDACATEROL MALEATE:Neuropathy peripheral:0.12), (INFLIXIMAB-DYYB:Neuropathy peripheral:0.36), (INSULIN ASPARTINSULIN DEGLUDEC:Neuropathy peripheral:0.14), (INSULIN PORK\INSULIN PURIFIED PORK:Neuropathy peripheral:0.41), (INTERFERON:Neuropathy peripheral:1.89), (INTERFERON ALFA-2A:Neuropathy peripheral:1.23), (INTERFERON ALFA-2B:Neuropathy peripheral:1.45), (INTERFERON GAMMA-1B:Neuropathy peripheral:1.26), (IRON POLYMALTOSE:Neuropathy peripheral:0.21), (IRONVITAMINS:Neuropathy peripheral:0.57), (ISONIAZIDPYRAZINAMIDERIFAMPIN:Neuropathy peripheral:0.03), (ITOPRIDE:Neuropathy peripheral:0.33), (KALLIDINOGENASE:Neuropathy peripheral:0.51), (LACIDIPINE:Neuropathy peripheral:0.12), (LANDIOLOL HYDROCHLORIDE:Neuropathy peripheral:0.04), (LATANOPROSTTIMOLOL:Neuropathy peripheral:0.85), (LEVOBUNOLOL:Neuropathy peripheral:0.06), (LEVOBUNOLOL HYDROCHLORIDE:Neuropathy peripheral:0.18), (LINSIDOMINE:Neuropathy peripheral:0.2), (LUTETIUM LU-177:Neuropathy peripheral:0.47), (LYCOPENE:Neuropathy peripheral:0.02), (LYSOZYME HYDROCHLORIDE:Neuropathy peripheral:0.9), (MAGNESIUM PIDOLATE:Neuropathy peripheral:0.05), (MANIDIPINE HYDROCHLORIDE:Neuropathy peripheral:0.17), (MECLOZINE DIHYDROCHLORIDE:Neuropathy peripheral:0.08), (METFORMIN PAMOATE:Neuropathy peripheral:0.06), (METHENAMINE MANDELATE:Neuropathy peripheral:0.89), (METHOXY POLYETHYLENE GLYCOL-EPOETIN BETA:Neuropathy peripheral:0.88), (MILRINONE:Neuropathy peripheral:0.47), (MITIGLINIDE CALCIUM HYDRATEVOGLIBOSE:Neuropathy peripheral:0.58), (NALTREXONE HYDROCHLORIDE:Neuropathy peripheral:0.01), (NEFAZODONE:Neuropathy peripheral:0.45), (OLMESARTAN:Neuropathy peripheral:0.93), (OPIPRAMOL:Neuropathy peripheral:0.51), (PARATHYROID HORMONE:Neuropathy peripheral:0.89), (PASIREOTIDE:Neuropathy peripheral:0.7), (PEGZEREPOETIN ALFA:Neuropathy peripheral:0.47), (PENTOSTATIN:Neuropathy peripheral:0.94), (PERPHENAZINE:Neuropathy peripheral:0.37), (PHENPROCOUMON:Neuropathy peripheral:0.57), (PILSICAINIDE HYDROCHLORIDE:Neuropathy peripheral:1.76), (PIMOBENDAN:Neuropathy peripheral:1.13), (PIPERACILLIN:Neuropathy peripheral:0.42), (PIRBUTEROL ACETATE:Neuropathy peripheral:0.1), (POLYMYXIN B SULFATETRIMETHOPRIM SULFATE:Neuropathy peripheral:0.04), (POLYSTYRENE:Neuropathy peripheral:0.33), (POLYSTYRENE SULFONIC ACID:Neuropathy peripheral:0.5), (POTASSIUM CHLORIDE\SODIUM CHLORIDE\SODIUM LACTATE:Neuropathy peripheral:0.3), (POTASSIUM IODIDE:Neuropathy peripheral:0.96), (PRANLUKAST:Neuropathy peripheral:1.5), (PREDNISOLONE SODIUM PHOSPHATE:Neuropathy peripheral:1.53), (PRISTINAMYCIN:Neuropathy peripheral:0.07), (PROCATEROL HYDROCHLORIDE:Neuropathy peripheral:0.74), (PROPATYL NITRATE:Neuropathy peripheral:0.09), (PROPIVERINE:Neuropathy peripheral:0.48), (PROTRIPTYLINE HYDROCHLORIDE:Neuropathy peripheral:1.54), (PYRIDOXAL PHOSPHATE:Neuropathy peripheral:0.35), (PYRIMETHAMINE:Neuropathy peripheral:0.07), (QUINACRINE:Neuropathy peripheral:1.2), (RANIMUSTINE:Neuropathy peripheral:0.08), (RETINOL:Neuropathy peripheral:1.43), (RILMENIDINE PHOSPHATE:Neuropathy peripheral:0.38), (RISEDRONIC ACID:Neuropathy peripheral:0.11), (RUFINAMIDE:Neuropathy peripheral:0.12), (SELEGILINE HYDROCHLORIDE:Neuropathy peripheral:0.27), (SIBUTRAMINE HYDROCHLORIDE:Neuropathy peripheral:0.61), (SITAFLOXACIN:Neuropathy peripheral:0.37), (SODIUM CITRATE:Neuropathy peripheral:0.46), (SODIUM PHOSPHATE, DIBASIC\SODIUM PHOSPHATE, MONOBASIC:Neuropathy peripheral:1.38), (SOFOSBUVIRVELPATASVIR:Neuropathy peripheral:0.93), (ST. JOHN'S WORT:Neuropathy peripheral:0.26), (STREPTOMYCIN SULFATE:Neuropathy peripheral:0.05), (STREPTOMYCIN\STREPTOMYCIN SULFATE:Neuropathy peripheral:0.46), (SUFENTANIL CITRATE:Neuropathy peripheral:0.19), (SULFACETAMIDE SODIUMSULFUR:Neuropathy peripheral:0.02), (SULTOPRIDE HYDROCHLORIDE:Neuropathy peripheral:0.29), (SUPLATAST TOSILATE:Neuropathy peripheral:1.17), (TAZOBACTAM:Neuropathy peripheral:0.77), (TEDUGLUTIDE:Neuropathy peripheral:0.24), (TEDUGLUTIDE\WATER:Neuropathy peripheral:1.23), (TEGAFURURACIL:Neuropathy peripheral:0.34), (TELOTRISTAT ETHYL:Neuropathy peripheral:0.7), (TEMSIROLIMUS:Neuropathy peripheral:0.47), (TERBUTALINE:Neuropathy peripheral:0.77), (TESTOSTERONE UNDECANOATE:Neuropathy peripheral:0.99), (TETRABENAZINE:Neuropathy peripheral:0.55), (THEOPHYLLINE:Neuropathy peripheral:0.92), (THIOTHIXENE:Neuropathy peripheral:0.15), (TIBOLONE:Neuropathy peripheral:0.33), (TIGECYCLINE:Neuropathy peripheral:0.68), (TIMOLOL MALEATETRAVOPROST:Neuropathy peripheral:1.04), (TIMOLOLTRAVOPROST:Neuropathy peripheral:0.54), (TIOPRONIN:Neuropathy peripheral:0.09), (TOSUFLOXACIN TOSYLATE:Neuropathy peripheral:0.73), (TREPROSTINIL DIOLAMINE:Neuropathy peripheral:0.56), (TRIMIPRAMINE MALEATE:Neuropathy peripheral:0.73), (TRIPTORELIN PAMOATE:Neuropathy peripheral:0.76), (TROXIPIDE:Neuropathy peripheral:0.22), (UROKINASE:Neuropathy peripheral:0.14), (VALRUBICIN:Neuropathy peripheral:0.22), (VANDETANIB:Neuropathy peripheral:0.29), (VONOPRAZAN:Neuropathy peripheral:0.31), (ZILEUTON:Neuropathy peripheral:1.28), (ZOTEPINE:Neuropathy peripheral:0.23), (ACETAMINOPHENOXYCODONEOXYCODONE HYDROCHLORIDE:Neutropenia:0.62), (ACETAMINOPHENPROPOXYPHENE HYDROCHLORIDE:Neutropenia:0.31), (ACETAMINOPHENPROPOXYPHENE NAPSYLATE:Neutropenia:0.09), (AGALSIDASE BETA:Neutropenia:0.29), (ALDESLEUKIN:Neutropenia:0.63), (ALUMINUM HYDROXIDEMAGNESIUM OXIDEOXETHAZAINE:Neutropenia:0.46), (AMANTADINE:Neutropenia:0.35), (AMINOSAL ICYLIC ACID:Neutropenia:0.06), (AMISULPRIDE:Neutropenia:0.58), (AMLODIPINE BESYLATEHYDROCHLOROTHIAZIDEVALSARTAN:Neutropenia:0.09), (AMLODIPINE VALSARTAN:Neutropenia:0.46), (AMMONIUM LACTATE:Neutropenia:0.2), (ANTIHISTAMINES NOS:Neutropenia:0.2), (ARMODAFINIL:Neutropenia:0.12), (ARSENIC TRIOXIDE:Neutropenia:0.04), (AXITINIB:Neutropenia:0.57), (AZELASTINE:Neutropenia:0.07), (BELATACEPT:Neutropenia:0.42), (BENAZEPRIL HYDROCHLORIDE:Neutropenia:1.12), (BILASTINE:Neutropenia:0.17), (BRINZOLAMIDE:N eutropenia:0.92), (CABOZANTINIB:Neutropenia:0.11), (CABOZANTINIB S-MALATE:Neutropenia:0.04), (CALCIUM\MAGNESIUM:Neutropenia:0.03), (CANAKINUMA B:Neutropenia:0.68), (CARBIMAZOLE:Neutropenia:0.48), (CARMUSTINE:Neutropenia:1), (C ARVEDILOL PHOSPHATE:Neutropenia:0.7), (CASANTHRANOLDOCUSATE SODIUM:Neutropenia:0.28), (CEFOTAXIMECEFOTAXIME SODIUM:Neutropenia:0.87), (CERTOPARIN SODIUM:Neutropenia:0.02), (CHONDROITIN SULFATE (CHICKEN):Neutropenia:0.23), (CICLESONIDE:Neutropenia:0.31), (CLOBAZAM:Neutropenia: 0.58), (COLISTIN:Neutropenia:0.57), (CYCLOPHOSPHAMIDECYTARABINEDEXAMETH ASONEDOXORUBICINMETHOTREXATEVINCRISTINE:Neutropenia:0.12), (CYPROHEP TADINE:Neutropenia:0.98), (CYPROHEPTADINE HYDROCHLORIDE:Neutropenia:0.21), (DABRAFENIB MESYLATE:Neutropenia:0.48), (DACLIZUMAB:Neutropenia:1.2), (DAUNORUBICIN HYDROCHLORIDE:Neutropenia:0.68), (DEFERIPRONE:Neutropenia:0.18), (DEXLANSOPR AZOLE:Neutropenia:0.81), (DIGITOXIN:Neutropenia:0.75), (DIPYRIDAMOLE:Neutropenia:0.96), (DORZOLAMIDE:Neutropenia:0.35), (DRONEDARONE:Neutropenia:0.05), (EDOXABAN TOSYLATE:Neutropenia:0.95), (ELDECALCITOL:Neutropenia:0.11), (ELETRIPTAN HYDROBROMIDE:Neutropenia:0.55), (EPINASTINE HYDROCHLORIDE:Neutropenia:0.04), (EVOLOCUMAB:Neutropenia:0.13), (FAMCICLOVIR: Neutropenia:0.01), (FENOTEROL HYDROBROMIDE:Neutropenia:0.26), (FERROUS SULFATEFOLIC ACID:Neutropenia:0.69), (FERROUS SULFATEGLYCINE:Neutropenia:0.33), (FESOTERODINE FUMARATE:Neutropenia:0.93), (FLUOCINONIDE:Neutropenia:0.21), (FLUOROURACILLE UCOVORIN CALCIUMOXALIPLATIN:Neutropenia:0.11), (FLUTICASONE:Neutropenia:0.86), (FLUVAS TATIN SODIUM:Neutropenia:0.42), (FORMOTEROL FUMARATE DIHYDRATE\MOMETASONE FUROATE:Neutropenia:0.69), (FORMOTEROL FUMARATE\MOMETASONE FUROATE:Neutropenia:0.06), (FOSPHENYTOINFOSPHENYTOIN SODIUM:Neutropenia:0.42), (GEFITINIB:Neutropenia:0.68), (GIMERACILOTERACILTEGA FUR:Neutropenia:0.96), (GLYCOPYRRONIUM:Neutropenia:0.17), (GLYCOSAMINOGLYCA NS:Neutropenia:0.3), (GUSPERIMUS TRIHYDROCHLORIDE:Neutropenia:0.03), (HERBALSSAW PALMETTO:Neutropenia:0.16), (HYDROCORTISONE ACETATE:Neutropenia:0.86), (HYDROCORTISONE HEMISUCCINATE:Neutropenia:0.05), (HYDROXYCHLOROQUINE DIPHOSPHATE:Neutropenia:0), (ILOPROST:Neutropenia:0.32), (IMIPRAMINE HYDROCHLORIDE:Neutropenia:0.55), (INTERFERON ALFA-2A:Neutropenia:0.94), (INTERFERON ALFA-2A OR INTERFERON ALFA-2B:Neutropenia:0.25), (INTERFERON ALFA-2B:Neutropenia:0.79), (INTERFERON GAMMA-1B:Neutropenia:0.66), (ISOSORBIDE:Neutropenia:0.79), (LAPATINIB:Neutropenia:0.24), (LAP ATINIB DITOSYLATE:Neutropenia:0.72), (LENVATINIB:Neutropenia:0.48), (LERCANIDIPINE HYDROCHLORIDE:Neutropenia:1.55), (LEVALBUTEROL HYDROCHLORIDE:Neutropenia:0.95), (LOVASTATIN:Neutropenia:0.88), (MAGNESIUM ASPARTATE HYDROCHLORIDE:Neutropenia:0.15), (MAGNESIUM ASPARTATE\POTASSIUM ASPARTATE:Neutropenia:0.32), (MECLIZINEMECLIZINE HYDROCHLORIDE:Neutropenia:0.49), (METAXALONE:Neutropenia:0.14), (METHENOLON E ENANTHATE:Neutropenia:0.91), (METHYLPREDNISOLONE ACETATE:Neutropenia:0.98), (METOLAZONE:Neutropenia:0.34), (NABUMETONE:Neutrope nia:0.25), (NADOLOL:Neutropenia:0.27), (NORTRIPTYLINE:Neutropenia:0.43), (OLANZAPIN E:Neutropenia:1.22), (OMEGA-3-ACID ETHYL ESTERS:Neutropenia:0.84), (OSIMERTINIB:Neutropenia:0.35), (OXYBUTYNIN CHLORIDE:Neutropenia:0.63), (PARICALCITOL:Neutropenia:0.17), (PENICILLIN G:Neutropenia:0.59), (POLYMYXIN B SULFATE:Neutropenia:0.11), (PORFIMER SODIUM:Neutropenia:0.34), (PRAMIPEXOLE DIHYDROCHLORIDE:Neutropenia:0.37), (PREDNISOLONE SODIUM PHOSPHATE:Neutropenia:1.25), (PRIMIDONE:Neutropenia:1.02), (PRISTINAMYCIN:Neutro penia:0.32), (PYRIMETHAMINE:Neutropenia:0.16), (RALOXIFENE HYDROCHLORIDE:Neutropenia:0.27), (RAMUCIRUMAB:Neutropenia:1.05), (RANOLAZIN E:Neutropenia:0.77), (RILMENIDINE PHOSPHATE:Neutropenia:0.15), (RIVASTIGMINE TARTRATE:Neutropenia:0.08), (RIZATRIPTAN BENZOATE:Neutropenia:0.17), (ROFLUMILAST:Neutropenia:0.82), (ROPINIROLE HYDROCHLORIDE:Neutropenia:0.28), (SECUKINUMAB:Neutropenia:0.69), (SIMEPREVIR: Neutropenia:0.02), (SIPULEUCEL-T:Neutropenia:0.03), (SODIUM CITRATE:Neutropenia:0.24), (SODIUM PHOSPHATE, DIBASIC\SODIUM PHOSPHATE, MONOBASIC:Neutropenia:0.06), (SUMATRIPTANSUMATRIPTAN SUCCINATE:Neutropenia:0.09), (TAMOXIFEN CITRATE:Neutropenia:0.4), (TEDUGLUTIDE\WATER:Neutropenia:0.06), (TENIPOSIDE:Neut ropenia:0.2), (TERAZOSIN HYDROCHLORIDE:Neutropenia:0.18), (TERBUTALINE SULFATE:Neutropenia:1.16), (TETRACYCLINE:Neutropenia:0.29), (THIOGUANINE:Neutrop enia:0.19), (TISAGENLECLEUCEL:Neutropenia:0.1), (TIZANIDINE:Neutropenia:0.54), (TOCO PHEROL:Neutropenia:0.09), (TOLTERODINE TARTRATE:Neutropenia:0.53), (TROSPIUM CHLORIDE:Neutropenia:0.29), (ZIV-AFLIBERCEPT:Neutropenia:0.55), (ADO-TRASTUZUMAB EMTANSINE:Neutrophil count decreased:0.1), (ALENDRONIC ACID:Neutrophil count decreased:0.03), (AMIODARONE HYDROCHLORIDE:Neutrophil count decreased:0.12), (ANASTROZOLE:Neutrophil count decreased:0.34), (BUMETANIDE:Neutrophil count decreased:0.65), (CALCITRIOL:Neutrophil count decreased:0.19), (CARMUSTINE:Neutrophil count decreased:0.05), (CETUXIMAB:Neutrophil count decreased:0.33), (CILASTATIN SODIUMIMIPENEM:Neutrophil count decreased:0.14), (CITALOPRAM HYDROBROMIDE:Neutrophil count decreased:0.59), (CLINDAMYCINCLINDAMYCIN PHOSPHATE:Neutrophil count decreased:0.7), (CLONIDINE:Neutrophil count decreased:0.1), (DASATINIB:Neutrophil count decreased:0.69), (DEXAMETHASONE ACETATE:Neutrophil count decreased:0.05), (DEXLANSOPRAZOLE:Neutrophil count decreased:0.27), (DILTIAZEM:Neutrophil count decreased:0.39), (DIVALPROEX SODIUM:Neutrophil count decreased:0.04), (ENALAPRIL:Neutrophil count decreased:1.14), (EVEROLIMUS:Neutrophil count decreased:0.44), (FERROUS SULFATE:Neutrophil count decreased:0.08), (FERROUS SULFATEFERROUS SULFATE, DRIED:Neutrophil count decreased:0.71), (FLUTICASONE PROPIONATE:Neutrophil count decreased:0.49), (IRON:Neutrophil count decreased:0.1), (LETROZOLE:Neutrophil count decreased:0.16), (LINAGLIPTIN:Neutrophil count decreased:0), (MOXIFLOXACIN HYDROCHLORIDE:Neutrophil count decreased:0.04), (NAPROXEN:Neutrophil count decreased:0.22), (NEBIVOLOL:Neutrophil count decreased:0.08), (NILOTINIB:Neutrophil count decreased:1.36), (NITROGLYCERIN:Neutrophil count decreased:0.13), (OXYGEN:Neutrophil count decreased:0.11), (PEMETREXED:Neutrophil count decreased:0.1), (SACUBITRILVALSARTAN:Neutrophil count decreased:0.93), (THALIDOMIDE:Neutrophil count decreased:1.45), (TINZAPARIN SODIUM:Neutrophil count decreased:0.08), (TOPIRAMATE:Neutrophil count decreased:0.28), (TRAZODONE HYDROCHLORIDE:Neutrophil count decreased:0.52), (ESOMEPRAZOLE MAGNESIUM:Nicotine dependence:0.32), (FOLIC ACID:Nicotine dependence:0.37), (NAPROXEN:Nicotine dependence:0.01), (ARIPIPRAZOLE:Night sweats:0.02), (CALCIUM CARBONATECHOLECALCIFEROL:Night sweats:0.17), (CERTOLIZUMAB PEGOL:Night sweats:0.22), (DEXLANSOPRAZOLE:Night sweats:0.37), (DICLOFENAC SODIUM:Night sweats:0.11), (DILTIAZEM HYDROCHLORIDE:Night sweats:0.21), (DIVALPROEX SODIUM:Night sweats:0.51), (DOCUSATE SODIUM:Night sweats:0.67), (ESZOPICLONE:Night sweats:0.03), (EVOLOCUMAB:Night sweats:0.31), (LISDEXAMFETAMINE DIMESYLATE:Night sweats:0.26), (METAXALONE:Night sweats:0.03), (MODAFINIL:Night sweats:0.04), (OXCARBAZEPINE:Night sweats:0), (SACUBITRILVALSARTAN:Night sweats:0.12), (SUMATRIPTANSUMATRIPTAN SUCCINATE:Night sweats:0.01), (ARMODAFINIL:Nightmare:0.94), (BUPRENORPHINE HYDROCHLORIDE:Nightmare:0.31), (CERTOLIZUMAB PEGOL:Nightmare:0.42), (DALFAMPRIDINE:Nightmare:0.24), (DEXLANSOPRAZOLE:Night mare:0.71), (DICLOFENAC SODIUM:Nightmare:0.08), (DILTIAZEM HYDROCHLORIDE:Nightmare:0), (ELETRIPTAN HYDROBROMIDE:Nightmare:0.28), (ENALAPRIL:Nightmare:0.42), (ESZOPICLONE:Nightm are:0.56), (EVOLOCUMAB:Nightmare:0.26), (FERROUS SULFATEFERROUS SULFATE, DRIED:Nightmare:0.24), (HALOPERIDOL:Nightmare:0.51), (HYOSCYAMINE:Nightmare:0.07), (LEVALBUTEROL HYDROCHLORIDE:Nightmare:0.01), (OXCARBAZEPINE:Nightmare:1.25), (PAROXETINE PAROXETINE HYDROCHLORIDE:Nightmare:0.18), (POLYETHYLENE GLYCOLS:Nightmare:0.04), (RANOLAZINE:Nightmare:0.05), (UMECLIDINIUM BROMIDEVILANTEROL TRIFENATATE:Nightmare:0), (WARFARIN:Nightmare:0.14), (WARFARIN SODIUM:Nightmare:0.05), (ALBUTEROL SULFATEIPRATROPIUM BROMIDE:Nocturia:0.19), (AMIODARONE HYDROCHLORIDE:Nocturia:0.05), (AMPHETAMINE ASPARTATEAMPHETAMINE SULFATEDEXTROAMPHETAMINE SACCHARATEDEXTROAMPHETAMINE SULFATE:Nocturia:0.18), (CALCIUM CITRATE:Nocturia:0.06), (CANDESARTAN CILEXETIL:Nocturia:0.36), (CARVEDILOL PHOSPHATE:Nocturia:0.14), (CIPROFLOXACIN HYDROCHLORIDE:Nocturia:0.04), (DICLOFENAC:Nocturia:0.56), (DIPHENHYDRAMINE: Nocturia:0.13), (DIVALPROEX SODIUM:Nocturia:0.22), (DULOXETINE:Nocturia:0.32), (EVOLOCUMAB:Nocturia:0.7), (FER ROUS SULFATEFERROUS SULFATE, DRIED:Nocturia:0.25), (FINGOLIMOD HYDROCHLORIDE:Nocturia:0.32), (HYDROCHLOROTHIAZIDEVALSARTAN:Nocturia:0.01), (INSULIN HUMAN:Nocturia:0.05), (METRONIDAZOLE:Nocturia:0.81), (MODAFINIL:Nocturia:0.05), (N EBIVOLOL:Nocturia:0.16), (OLANZAPINE:Nocturia:0.13), (OMEGA-3-ACID ETHYL ESTERS:Nocturia:0.05), (OXYGEN:Nocturia:0.43), (PAROXETINE:Nocturia:0.07), (PRAMIPE XOLE:Nocturia:0.41), (SACUBITRILVALSARTAN:Nocturia:0.75), (SODIUM OXYBATE:Nocturia:0.04), (TIZANIDINE HYDROCHLORIDE:Nocturia:0.36), (TOFACITINIB CITRATE:Nocturia:0.1), (TORSEMIDE:Nocturia:0.2), (VARENICLINE TARTRATE:Nocturia:0.14), (VITAMIN B:Nocturia:0.28), (.ALPHA.1-PROTEINASE INHIBITOR HUMAN:Non-Hodgkin's lymphoma:0.27), (ACETAMINOPHENBUTALBITALCAFFEINE:Non-Hodgkin's lymphoma:1.1), (ACITRETIN:Non-Hodgkin's lymphoma:0), (ACLARUBICIN HYDROCHLORIDE:Non-Hodgkin's lymphoma:0.05), (ACLIDINIUM BROMIDE:Non-Hodgkin's lymphoma:0.02), (ADO-TRASTUZUMAB EMTANSINE:Non-Hodgkin's lymphoma:0.62), (AGALSIDASE BETA:Non-Hodgkin's lymphoma:0.26), (ALBUMIN HUMAN:Non-Hodgkin's lymphoma:0.01), (ALCOHOL:Non-Hodgkin's lymphoma:0.23), (ALDESLEUKIN:Non-Hodgkin's lymphoma:0.05), (ALFACALCIDOL:Non-Hodgkin's lymphoma:0.73), (ALISKIREN HEMIFUMARATE:Non-Hodgkin's lymphoma:0.01), (ALUMINUM HYDROXIDEDIMETHICONEMAGNESIUM HYDROXIDE:Non-Hodgkin's lymphoma:0.63), (ALUMINUM HYDROXIDEMAGNESIUM OXIDEOXETHAZAINE:Non-Hodgkin's lymphoma:0), (AMANTADINE:Non-Hodgkin's lymphoma:0.81), (AMIKACIN:Non-Hodgkin's lymphoma:0.32), (AMISULPRIDE:Non-Hodgkin's lymphoma:0.68), (AMLODIPINE BESYLATEHYDROCHLOROTHIAZIDEOLMESARTAN MEDOXOMIL:Non-Hodgkin's lymphoma:0.01), (AMLODIPINE BESYLATEHYDROCHLOROTHIAZIDEVALSARTAN:Non-Hodgkin's lymphoma:0.07), (AMLODIPINEPERINDOPRIL:Non-Hodgkin's lymphoma:0.27), (AMMONIUM LACTATE:Non-Hodgkin's lymphoma:0.03), (AMPICILLIN:Non-Hodgkin's lymphoma:0.23), (ARMODAFINIL:Non-Hodgkin's lymphoma:0.68), (AXITINIB:Non-Hodgkin's lymphoma:0.06), (AZACITIDINE:Non-Hodgkin's lymphoma:0.94), (BACITRACIN:Non-Hodgkin's lymphoma:0.14), (BENAZEPRILBENAZEPRIL HYDROCHLORIDE:Non-Hodgkin's lymphoma:0.55), (BRINZOLAMIDE:Non-Hodgkin's lymphoma:0.6), (BUDESONIDEFORMOTEROL FUMARATE:Non-Hodgkin's lymphoma:0.48), (BUPRENORPHINE HYDROCHLORIDE:Non-Hodgkin's lymphoma:0.42), (BUPRENORPHINE HYDROCHLORIDENALOXONE HYDROCHLORIDE:Non-Hodgkin's lymphoma:0.06), (CABAZITAXEL:Non-Hodgkin's lymphoma:0.11), (CALCITONIN:Non-Hodgkin's lymphoma:0.49), (CALCITONIN SALMON:Non-Hodgkin's lymphoma:0.61), (CANAKINUMAB:Non-Hodgkin's lymphoma:0.56), (CANDESARTAN CILEXETILHYDROCHLOROTHIAZIDE:Non-Hodgkin's lymphoma:1.49), (CARBIMAZOLE:Non-Hodgkin's lymphoma:0.01), (CARISOPRODOL:Non-Hodgkin's lymphoma:0.31), (CERTOLIZUMAB PEGOL:Non-Hodgkin's lymphoma:1.35), (CHLORDIAZEPOXIDE HYDROCHLORIDECLIDINIUM BROMIDE:Non-Hodgkin's lymphoma:0.1), (CHLORTHALIDONE:Non-Hodgkin's lymphoma:0.96), (CLINDAMYCIN PHOSPHATE:Non-Hodgkin's lymphoma:0.09), (CLOFARABINE:Non-Hodgkin's lymphoma:0.7), (COLESEVELAM HYDROCHLORIDE:Non-Hodgkin's lymphoma:0.38), (CYCLOPHOSPHAMIDECYTARABINEDEXAMETHASONEDOXORUB ICINMETHOTREXATEVINCRISTINE:Non-Hodgkin's lymphoma:0.09), (CYPROHEPTADINE:Non-Hodgkin's lymphoma:0.52), (DABIGATRAN:Non-Hodgkin's lymphoma:0.34), (DABRAFENIB:Non-Hodgkin's lymphoma:0.46), (DACLIZUMAB:Non-Hodgkin's lymphoma:0.78), (DECITABINE:Non-Hodgkin's lymphoma:0.85), (DEFLAZACORT:Non-Hodgkin's lymphoma:0.39), (DESONIDE:Non-Hodgkin's lymphoma:0.31), (DESVENLAFAXINE SUCCINATE:Non-Hodgkin's lymphoma:0.22), (DEVICE:Non-Hodgkin's lymphoma:0.06), (DIPYRIDAMOLE:Non-Hodgkin's lymphoma:0.2), (DOXEPIN HYDROCHLORIDE:Non-Hodgkin's lymphoma:0), (ECONAZOLE:Non-Hodgkin's lymphoma:0.64), (EDOXABAN TOSYLATE:Non-Hodgkin's lymphoma:0.74), (ELETRIPTAN HYDROBROMIDE:Non-Hodgkin's lymphoma:0.67), (EMTRICITABINE:Non-Hodgkin's lymphoma:0.04), (ESTROGENS, CONJUGATEDMEDROXYPROGESTERONE ACETATE:Non-Hodgkin's lymphoma:0.48), (FERROUS SULFATEFOLIC ACID:Non-Hodgkin's lymphoma:0), (FLUOCINONIDE:Non-Hodgkin's lymphoma:0.35), (FORMOTEROL:Non-Hodgkin's lymphoma:0.37), (FORMOTEROL FUMARATE:Non-Hodgkin's lymphoma:0.88), (FORMOTEROL FUMARATE\MOMETASONE FUROATE:Non-Hodgkin's lymphoma:0.2), (FOSFOMYCIN:Non-Hodgkin's lymphoma:0.03), (FULVESTRANT:Non-Hodgkin's lymphoma:0.03), (GEMTUZUMAB OZOGAMICIN:Non-Hodgkin's lymphoma:0.12), (GIMERACILOTERACILTEGAFUR:Non-Hodgkin's lymphoma:0.31), (GLYCOPYRRONIUMINDACATEROL:Non-Hodgkin's lymphoma:1.06), (HEPARIN CALCIUM:Non-Hodgkin's lymphoma:0.33), (HYDROCHLOROTHIAZIDETELMISARTAN:Non-Hodgkin's lymphoma:0.45), (HYDROCORTISONE ACETATE:Non-Hodgkin's lymphoma:0.04), (HYDROXYZINE PAMOATE:Non-Hodgkin's lymphoma:1.07), (HYOSCYAMINE:Non-Hodgkin's lymphoma:0.27), (ILOPROST:Non-Hodgkin's lymphoma:0.02), (IMIPENEM:Non-Hodgkin's lymphoma:0.33), (IMIPRAMINE HYDROCHLORIDE:Non-Hodgkin's lymphoma:0.23), (INTERFERON GAMMA-1B:Non-Hodgkin's lymphoma:0.13), (LAPATINIB DITOSYLATE:Non-Hodgkin's lymphoma:0.03), (LETROZOLE:Non-Hodgkin's lymphoma:0.57), (LISDEXAMFETAMINE DIMESYLATE:Non-Hodgkin's lymphoma:0.21), (MECLIZINEMECLIZINE HYDROCHLORIDE:Non-Hodgkin's lymphoma:0.39), (MEPOLIZUMAB:Non-Hodgkin's lymphoma:0.1), (METAXALONE:Non-Hodgkin's lymphoma:1.1), (METFORMIN HYDROCHLORIDEVILDAGLIPTIN:Non-Hodgkin's lymphoma:0.27), (METHENOLONE ENANTHATE:Non-Hodgkin's lymphoma:0.3), (METHIMAZOLE:Non-Hodgkin's lymphoma:0.35), (METHYLPHENIDATE HYDROCHLORIDE:Non-Hodgkin's lymphoma:0.37), (MORPHINE SULFATE\NALTREXONE HYDROCHLORIDE:Non-Hodgkin's lymphoma:0.06), (MYCOPHENOLATE MOFETILMYCOPHENOLATE MOFETIL HYDROCHLORIDE:Non-Hodgkin's lymphoma:1.29), (NILOTINIB:Non-Hodgkin's lymphoma:2.32), (NINTEDANIB:Non-Hodgkin's lymphoma:0.53), (NOREPINEPHRINE:Non-Hodgkin's lymphoma:0.09), (OLAPARIB:Non-Hodgkin's lymphoma:0.07), (OLODATEROL HYDROCHLORIDETIOTROPIUM BROMIDE MONOHYDRATE:Non-Hodgkin's lymphoma:0.34), (OMEGA-3-ACID ETHYL ESTERS:Non-Hodgkin's lymphoma:1.12), (OSELTAMIVIR:Non-Hodgkin's lymphoma:0.32), (PEGINTERFERON BETA-1A:Non-Hodgkin's lymphoma:0.04), (PEMETREXED:Non-Hodgkin's lymphoma:0.85), (PENICILLIN G:Non-Hodgkin's lymphoma:0.08), (PENTOXIFYLLINE:Non-Hodgkin's lymphoma:0.39), (PERTUZUMAB:Non-Hodgkin's lymphoma:0.1), (PHENOBARBITAL:Non-Hodgkin's lymphoma:0.79), (PHENYTOIN SODIUM:Non-Hodgkin's lymphoma:0.1), (PIMECROLIMUS:Non-Hodgkin's lymphoma:0.02), (PIROXICAM:Non-Hodgkin's lymphoma:0.38), (PONATINIB:Non-Hodgkin's lymphoma:1.6), (PROPAFENONE HYDROCHLORIDE:Non-Hodgkin's lymphoma:0.11), (PSYLLIUM HUSK:Non-Hodgkin's lymphoma:0.09), (QUINAPRIL:Non-Hodgkin's lymphoma:0.03), (REGORAFENIB:Non-Hodgkin's lymphoma:0.45), (RIVASTIGMINE:Non-Hodgkin's lymphoma:0.25), (RIVASTIGMINE TARTRATE:Non-Hodgkin's lymphoma:0.56), (RIZATRIPTAN BENZOATE:Non-Hodgkin's lymphoma:0.26), (ROFLUMILAST:Non-Hodgkin's lymphoma:0.58), (SACUBITRILVALSARTAN:Non-Hodgkin's lymphoma:0.55), (SECUKINUMAB:Non-Hodgkin's lymphoma:0.75), (SITAGLIPTIN:Non-Hodgkin's lymphoma:0.46), (SODIUM FLUORIDE:Non-Hodgkin's lymphoma:0.16), (SUMATRIPTAN:Non-Hodgkin's lymphoma:0.13), (SUMATRIPTAN SUCCINATE:Non-Hodgkin's lymphoma:0.15), (SUMATRIPTANSUMATRIPTAN SUCCINATE:Non-Hodgkin's lymphoma:0.62), (TERBUTALINE SULFATE:Non-Hodgkin's lymphoma:1.07), (TETRACYCLINE:Non-Hodgkin's lymphoma:0.99), (TIGECYCLINE:Non-Hodgkin's lymphoma:0.31), (TIZANIDINE:Non-Hodgkin's lymphoma:0.68), (TIZANIDINE HYDROCHLORIDE:Non-Hodgkin's lymphoma:0.97), (TRAMETINIB:Non-Hodgkin's lymphoma:0.31), (TRANEXAMIC ACID:Non-Hodgkin's lymphoma:0.19), (TRETINOIN:Non-Hodgkin's lymphoma:0.21), (TROSPIUM CHLORIDE:Non-Hodgkin's lymphoma:0.14), (VEMURAFENIB:Non-Hodgkin's lymphoma:0.24), (AMIODARONE HYDROCHLORIDE:Non-Hodgkin's lymphoma unspecified histology indolent:0.39), (CALCIUM CARBONATECHOLECALCIFEROL:Non-Hodgkin's lymphoma unspecified histology indolent:0.74), (CARFILZOMIB:Non-Hodgkin's lymphoma unspecified histology indolent:0.07), (CERTOLIZUMAB PEGOL:Non-Hodgkin's lymphoma unspecified histology indolent:0.41), (DASATINIB:Non-Hodgkin's lymphoma unspecified histology indolent:0.15), (DILTIAZEM:Non-Hodgkin's lymphoma unspecified histology indolent:0.47), (EPINEPHRINE:Non-Hodgkin's lymphoma unspecified histology indolent:0.44), (EVOLOCUMAB:Non-Hodgkin's lymphoma unspecified histology indolent:0.05), (FLECAINIDE:Non-Hodgkin's lymphoma unspecified histology indolent:0.08), (INDAPAMIDEPERINDOPRIL:Non-Hodgkin's lymphoma unspecified histology indolent:0.01), (IRON:Non-Hodgkin's lymphoma unspecified histology indolent:0.19), (ISOSORBIDE MONONITRATE:Non-Hodgkin's lymphoma unspecified histology indolent:0.06), (LERCANIDIPINE:Non-Hodgkin's lymphoma unspecified histology indolent:0.18), (MONTELUKAST SODIUM:Non-Hodgkin's lymphoma unspecified histology indolent:0.29), (NILOTINIB:Non-Hodgkin's lymphoma unspecified histology indolent:0.16), (PANTOPRAZOLE:Non-Hodgkin's lymphoma unspecified histology indolent:0.67), (PRAVASTATIN:Non-Hodgkin's lymphoma unspecified histology indolent:0.28), (PYRAZINAMIDE:Non-Hodgkin's lymphoma unspecified histology indolent:0.58), (SACUBITRILVALSARTAN:Non-Hodgkin's lymphoma unspecified histology indolent:0.52), (SOTALOL:Non-Hodgkin's lymphoma unspecified histology indolent:0.12), (THALIDOMIDE:Non-Hodgkin's lymphoma unspecified histology indolent:0.43), (TOPIRAMATE:Non-Hodgkin's lymphoma unspecified histology indolent:0.05), (TRAZODONE HYDROCHLORIDE:Non-Hodgkin's lymphoma unspecified histology indolent:0.24), (.ALPHA.-TOCOPHEROLRETINOL:Non-small cell lung cancer:0.2), (.ALPHA.1-PROTEINASE INHIBITOR HUMAN:Non-small cell lung cancer:0.42), (ABIRATERONE:Non-small cell lung cancer:0.43), (ACETAMINOPHENAPRONALIDECAFFEINEPROPYPHENAZONE:Non-small cell lung cancer:0.11), (ACETAMINOPHEN\CAFFEINEOPIUM:Non-small cell lung cancer:0.4), (ACETAMINOPHENPROPOXYPHENE NAPSYLATE:Non-small cell lung cancer:0.41), (ACTIVATED CHARCOAL:Non-small cell lung cancer:0.63), (ADENOSINE TRIPHOSPHATE:Non-small cell lung cancer:0.19), (AGALSIDASE BETA:Non-small cell lung cancer:0.87), (ALISKIREN HEMIFUMARATE:Non-small cell lung cancer:0.77), (ALOGLIPTIN:Non-small cell lung cancer:0.07), (ALUMINUM SILICATE:Non-small cell lung cancer:0.43), (AMANTADINE:Non-small cell lung cancer:0.21), (AMANTADINE HYDROCHLORIDE:Non-small cell lung cancer:0.5), (AMINO ACIDSCARBOHYDRATESMINERALSSOYBEAN OILVITAMINS:Non-small cell lung cancer:0.56), (AMINOSALICYLIC ACID:Non-small cell lung cancer:0.14), (AMLODIPINE BESYLATEHYDROCHLOROTHIAZIDEOLMESARTAN MEDOXOMIL:Non-small cell lung cancer:0.47), (AMLODIPINE BESYLATEHYDROCHLOROTHIAZIDEVALSARTAN:Non-small cell lung cancer:0.92), (AMOXAPINE:Non-small cell lung cancer:1.03), (AMPICILLIN SODIUM:Non-small cell lung cancer:0.59), (ANAGLIPTIN:Non-small cell lung cancer:0.19), (ANTIHISTAMINES NOS:Non-small cell lung cancer:0.88), (ASCORBIC ACIDBIOTINCYANOCOBALAMINFOLIC ACIDNIACINPANTOTHENIC ACIDPYRIDOXINE\RIBOFLAVINTHIAMINE:Non-small cell lung cancer:0.06), (ASPIRINCLOPIDOGREL:Non-small cell lung cancer:0.29), (ASPIRINLANSOPRAZOLE:Non-small cell lung cancer:0.79), (ATENOLOLCHLORTHALIDONE:Non-small cell lung cancer:0.35), (ATROPINE SULFATE:Non-small cell lung cancer:1.07), (AZITHROMYCIN OR METHOTREXATE SODIUM:Non-small cell lung cancer:0.02), (BAMIFYLLINE HYDROCHLORIDE:Non-small cell lung cancer:0.02), (BECLOMETHASONE DIPROPIONATEFORMOTEROL:Non-small cell lung cancer:0.39), (BENAZEPRILBENAZEPRIL HYDROCHLORIDE:Non-small cell lung cancer:1.61), (BEPRIDIL:Non-small cell lung cancer:1.43), (BETAMETHASONE DIPROPIONATECALCIPOTRIENE:Non-small cell lung cancer:0.53), (BETAMETHASONE DIPROPIONATECALCIPOTRIENECALCIPOTRIENE HYDRATE:Non-small cell lung cancer:0.32), (BETAMETHASONECALCIPOTRIENE:Non-small cell lung cancer:0.74), (BETAMIPRONPANIPENEM:Non-small cell lung cancer:0.02), (BIFIDOBACTERIUM LONGUM:Non-small cell lung cancer:0.02), (BOCOCIZUMAB:Non-small cell lung cancer:0.38), (BOSUTINIB:Non-small cell lung cancer:0.46), (BUPRENORPHINE HYDROCHLORIDENALOXONE HYDROCHLORIDE:Non-small cell lung cancer:0.13), (CALCIPOTRIENE:Non-small cell lung cancer:0.64), (CALCIUM ACETATE:Non-small cell lung cancer:1.8), (CALCIUM CARBONATEERGOCALCIFEROLRETINOL:Non-small cell lung cancer:0.57), (CALCIUM\MAGNESIUM:Non-small cell lung cancer:0.92), (CALCIUM\MAGNESIUM\ZINC:Non-small cell lung cancer:0.13), (CAMPHORMENTHOLMETHYL SALICYLATE:Non-small cell lung cancer:0.45), (CANAGLIFLOZINMETFORMIN HYDROCHLORIDE:Non-small cell lung cancer:0.99), (CANAKINUMAB:Non-small cell lung cancer:0.89), (CARFILZOMIB:Non-small cell lung cancer:1.67), (CARTEOLOL HYDROCHLORIDE:Non-small cell lung cancer:0.61), (CARVEDILOL PHOSPHATE:Non-small cell lung cancer:2.18), (CASPOFUNGIN ACETATE:Non-small cell lung cancer:0.55), (CEFPROZIL:Non-small cell lung cancer:0.13), (CEFTAZIDIME SODIUM:Non-small cell lung cancer:0.01), (CELLULASEDIASTASELIPASE:Non-small cell lung cancer:0.43), (CEPHARANTHINE:Non-small cell lung cancer:0.77), (CERTOLIZUMAB PEGOL:Non-small cell lung cancer:1.53), (CHLORAMBUCIL:Non-small cell lung cancer:0.71), (CHLORDIAZEPOXIDE HYDROCHLORIDECLIDINIUM BROMIDE:Non-small cell lung cancer:0.51), (CLENBUTEROL HYDROCHLORIDE:Non-small cell lung cancer:0.57), (CLONIDINE HYDROCHLORIDE:Non-small cell lung cancer:1.1), (COLESTILAN CHLORIDE:Non-small cell lung cancer:0.12), (COLISTIMETHATE SODIUM:Non-small cell lung cancer:0.24), (COLISTIN:Non-small cell lung cancer:0.3), (CORTICOTROPIN:Non-small cell lung cancer:0.35), (CORTISONE ACETATE:Non-small cell lung cancer:0.15), (CYAMEMAZINE:Non-small cell lung cancer:0.54), (CYPROTERONE ACETATE:Non-small cell lung cancer:0.02), (DACLIZUMAB:Non-small cell lung cancer:0.59), (DANAZOL:Non-small cell lung cancer:0.65), (DANTROLENE SODIUM:Non-small cell lung cancer:0.44), (DAPAGLIFLOZIN PROPANEDIOL:Non-small cell lung cancer:0.41), (DEFLAZACORT:Non-small cell lung cancer:1.24), (DEGARELIX:Non-small cell lung cancer:1), (DESVENLAFAXINE SUCCINATE:Non-small cell lung cancer:0.51), (DEVICE:Non-small cell lung cancer:0.16), (DEXTRAN:Non-small cell lung cancer:0.08), (DEXTROAMPHETAMINE SULFATE:Non-small cell lung cancer:0.14), (DICLOFENAC SODIUMMISOPROSTOL:Non-small cell lung cancer:0.26), (DILAZEP:Non-small cell lung cancer:0.76), (DIPHENIDOL HYDROCHLORIDE:Non-small cell lung cancer:0.73), (DISOPYRAMIDE:Non-small cell lung cancer:0.29), (DOBUTAMINE HYDROCHLORIDE:Non-small cell lung cancer:0.88), (DORIPENEM:Non-small cell lung cancer:0.11), (DOXEPIN HYDROCHLORIDE:Non-small cell lung cancer:0.82), (EFINACONAZOLE:Non-small cell lung cancer:0.06), (ELCATONIN:Non-small cell lung cancer:0.48), (ELETRIPTAN HYDROBROMIDE:Non-small cell lung cancer:1.74), (ELOTUZUMAB:Non-small cell lung cancer:1.13), (EMPAGLIFLOZINLINAGLIPTIN:Non-small cell lung cancer:0.3), (EPIRUBICIN HYDROCHLORIDE:Non-small cell lung cancer:0.53), (ESTROGENS, CONJUGATEDMEDROXYPROGESTERONE ACETATE:Non-small cell lung cancer:1.2), (ETHYL ICOSAPENTATEICOSAPENT:Non-small cell lung cancer:0.55), (ETRETINATE:Non-small cell lung cancer:2.1), (EVOLOCUMAB:Non-small cell lung cancer:0.88), (FAROPENEM SODIUM:Non-small cell lung cancer:0.61), (FENOFIBRIC ACID:Non-small cell lung cancer:0.69), (FERROUS SULFATEGLYCINE:Non-small cell lung cancer:0.94), (FLAVIN ADENINE DINUCLEOTIDE:Non-small cell lung cancer:0.81), (FLUOROURACILIRINOTECANLEUCOVORIN:Non-small cell lung cancer:0.34), (FLUTAMIDE:Non-small cell lung cancer:0.69), (FORMOTEROL FUMARATE\MOMETASONE FUROATE:Non-small cell lung cancer:0.82), (FOSFLUCONAZOLE:Non-small cell lung cancer:0.01), (FOSFOMYCIN CALCIUM:Non-small cell lung cancer:0.13), (FOSINOPRIL:Non-small cell lung cancer:0.3), (FOSPHENYTOINFOSPHENYTOIN SODIUM:Non-small cell lung cancer:0.3), (FURSULTIAMINE:Non-small cell lung cancer:0.44), (GADOVERSETAMIDE:Non-small cell lung cancer:0.05), (GLYCOPYRROLATEINDACATEROL MALEATE:Non-small cell lung cancer:0.75), (HYDROCODONE BITARTRATEIBUPROFEN:Non-small cell lung cancer:0.39), (HYOSCYAMINE:Non-small cell lung cancer:0.74), (IBUDILAST:Non-small cell lung cancer:0), (IDARUCIZUMAB:Non-small cell lung cancer:0.17), (IDELALISIB:Non-small cell lung cancer:0.44), (ILOPROST:Non-small cell lung cancer:0.82), (IMIGLUCERASE:Non-small cell lung cancer:0.12), (IMIQUIMOD:Non-small cell lung cancer:0.13), (INFLIXIMAB-DYYB:Non-small cell lung cancer:0.68), (INOTUZUMAB OZOGAMICIN:Non-small cell lung cancer:0.21), (INTERFERON:Non-small cell lung cancer:0.6), (INTERFERON ALFA-2B:Non-small cell lung cancer:1.39), (INTERFERON ALFA-N1:Non-small cell lung cancer:0.3), (IRONPOLYSACCHARIDES:Non-small cell lung cancer:0.14), (ITRACONAZOLE\SODIUM CHLORIDE:Non-small cell lung cancer:0.12), (IXAZOMIB:Non-small cell lung cancer:0.47), (LANREOTIDE:Non-small cell lung cancer:0.45), (LANREOTIDE ACETATE:Non-small cell lung cancer:0.29), (LEDIPASVIRSOFOSBUVIR:Non-small cell lung cancer:1.21), (LEVOCARNITINE HYDROCHLORIDE:Non-small cell lung cancer:0.48), (LEVOLEUCOVORIN CALCIUM:Non-small cell lung cancer:2.05), (LINSIDOMINE:Non-small cell lung cancer:0.48), (LIOTHYRONINE:Non-small cell lung cancer:0.5), (LIOTHYRONINE SODIUM:Non-small cell lung cancer:0.85), (LISDEXAMFETAMINE DIMESYLATE:Non-small cell lung cancer:0.34), (LITHIUM:Non-small cell lung cancer:0.01), (LOMITAPIDE MESYLATE:Non-small cell lung cancer:0.15), (LOMUSTINE:Non-small cell lung cancer:0.21), (LUTETIUM LU-177:Non-small cell lung cancer:0.13), (MAGNESIUM ASPARTATE HYDROCHLORIDE:Non-small cell lung cancer:0.57), (MAGNESIUM PIDOLATE:Non-small cell lung cancer:0.35), (MANIDIPINE HYDROCHLORIDE:Non-small cell lung cancer:0.11), (MENATETRENONE:Non-small cell lung cancer:0.76), (MEPENZOLATE BROMIDE:Non-small cell lung cancer:0.12), (MEPOLIZUMAB:Non-small cell lung cancer:0.5), (METFORMIN HYDROCHLORIDESAXAGLIPTIN:Non-small cell lung cancer:0.23), (METFORMIN HYDROCHLORIDESAXAGLIPTIN HYDROCHLORIDE:Non-small cell lung cancer:0.2), (METHENOLONE ENANTHATE:Non-small cell lung cancer:1.47), (METHOXY POLYETHYLENE GLYCOL-EPOETIN BETA:Non-small cell lung cancer:0.02), (MEXILETINE:Non-small cell lung cancer:0.12), (MIDODRINE HYDROCHLORIDE:Non-small cell lung cancer:0.6), (MIZORIBINE:Non-small cell lung cancer:0.81), (MOEXIPRIL HYDROCHLORIDE:Non-small cell lung cancer:0.36), (NAFAMOSTAT MESYLATE:Non-small cell lung cancer:0.18), (NALBUPHINE HYDROCHLORIDE:Non-small cell lung cancer:0.17), (NEUROTROPIN:Non-small cell lung cancer:0.11), (NIRAPARIB:Non-small cell lung cancer:0.98), (OBINUTUZUMAB:Non-small cell lung cancer:0.22), (OFATUMUMAB:Non-small cell lung cancer:0.01), (OPIPRAMOL:Non-small cell lung cancer:0.51), (ORPHENADRINE:Non-small cell lung cancer:0.76), (PASIREOTIDE:Non-small cell lung cancer:0.25), (PATIROMER:Non-small cell lung cancer:0.14), (PAZUFLOXACIN MESILATE:Non-small cell lung cancer:0.28), (PEGINTERFERON ALFA-2B:Non-small cell lung cancer:0.21), (PEGZEREPOETIN ALFA:Non-small cell lung cancer:0.24), (PENICILLIN V:Non-small cell lung cancer:0.62), (PENICILLIN V POTASSIUM:Non-small cell lung cancer:0.16), (PENTAMIDINE ISETHIONATE:Non-small cell lung cancer:0.34), (PENTAZOCINE:Non-small cell lung cancer:0.09), (PENTOSTATIN:Non-small cell lung cancer:0.56), (PIMOBENDAN:Non-small cell lung cancer:1.08), (POLYETHYLENE GLYCOL 3350POTASSIUM CHLORIDE\SODIUM BICARBONATE\SODIUM CHLORIDE\SODIUM SULFATE ANHYDROUS:Non-small cell lung cancer:0.42), (POLYSTYRENE SULFONIC ACID:Non-small cell lung cancer:0.34), (POMALIDOMIDE:Non-small cell lung cancer:0.42), (POSACONAZOLE:Non-small cell lung cancer:0.42), (POTASSIUM CHLORIDE\SODIUM CHLORIDE\SODIUM LACTATE:Non-small cell lung cancer:0.1), (POTASSIUM CITRATETRISODIUM CITRATE DIHYDRATE:Non-small cell lung cancer:1.2), (POVIDONE:Non-small cell lung cancer:0.28), (PREDNISOLONE SODIUM PHOSPHATE:Non-small cell lung cancer:0.48), (PRISTINAMYCIN:Non-small cell lung cancer:0.69), (RADIUM RA-223 DICHLORIDE:Non-small cell lung cancer:0.65), (RALTITREXED:Non-small cell lung cancer:0.12), (RILPIVIRINE HYDROCHLORIDE:Non-small cell lung cancer:0.06), (RISEDRONIC ACID:Non-small cell lung cancer:0.08), (RIVASTIGMINE TARTRATE:Non-small cell lung cancer:1.09), (RIZATRIPTAN BENZOATE:Non-small cell lung cancer:1.07), (ROXATIDINE ACETATE HYDROCHLORIDE:Non-small cell lung cancer:0.05), (RUCAPARIB CAMSYLATE:Non-small cell lung cancer:0.11), (SACUBITRILVALSARTAN:Non-small cell lung cancer:0.06), (SARPOGRELATE HYDROCHLORIDE:Non-small cell lung cancer:0.62), (SAXAGLIPTIN HYDROCHLORIDE:Non-small cell lung cancer:0.47), (SECUKINUMAB:Non-small cell lung cancer:1.07), (SENNOSIDE B:Non-small cell lung cancer:0.04), (SERRAPEPTASE:Non-small cell lung cancer:0.02), (SEVELAMER CARBONATE:Non-small cell lung cancer:0.35), (SEVELAMER HYDROCHLORIDE:Non-small cell lung cancer:0.54), (SIPULEUCEL-T:Non-small cell lung cancer:0.54), (SODIUM GUALENATE MONOHYDRATE:Non-small cell lung cancer:0.66), (SODIUM OXYBATE:Non-small cell lung cancer:0.68), (SOFALCONE:Non-small cell lung cancer:0.01), (SORBITOL:Non-small cell lung cancer:0.28), (STARCH, WHEAT:Non-small cell lung cancer:0.18), (TABALUMAB:Non-small cell lung cancer:0.02), (TAFAMIDIS MEGLUMINE:Non-small cell lung cancer:0.18), (TECELEUKIN:Non-small cell lung cancer:0.37), (TEDUGLUTIDE\WATER:Non-small cell lung cancer:0.31), (TELOTRISTAT ETHYL:Non-small cell lung cancer:0.01), (TEMSIROLIMUS:Non-small cell lung cancer:1), (TESTOSTERONE UNDECANOATE:Non-small cell lung cancer:0.7), (TIMOLOLTRAVOPROST:Non-small cell lung cancer:0.04), (TIPEPIDINE HIBENZATE:Non-small cell lung cancer:0.82), (TIPIRACIL HYDROCHLORIDETRIFLURIDINE:Non-small cell lung cancer:1.41), (TOFACITINIB CITRATE:Non-small cell lung cancer:0.14), (TOLTERODINE:Non-small cell lung cancer:0.11), (TOSUFLOXACIN TOSYLATE:Non-small cell lung cancer:0.58), (TRABECTEDIN:Non-small cell lung cancer:0.29), (TRIHEXYPHENIDYL:Non-small cell lung cancer:0.12), (TRIMIPRAMINE MALEATE:Non-small cell lung cancer:0.62), (TRIPTORELIN PAMOATE:Non-small cell lung cancer:0.37), (VALDECOXIB:Non-small cell lung cancer:0.02), (VALGANCICLOVIR HYDROCHLORIDE:Non-small cell lung cancer:1.46), (VISMODEGIB:Non-small cell lung cancer:0.02), (VONOPRAZAN:Non-small cell lung cancer:0.52), (XIPAMIDE:Non-small cell lung cancer:0.32), (ZILEUTON:Non-small cell lung cancer:0.47), (ZIV-AFLIBERCEPT:Non-small cell lung cancer:1.04), (ACETAMINOPHENCODEINE PHOSPHATE:Non-small cell lung cancer metastatic:0.51), (AMIODARONE HYDROCHLORIDE:Non-small cell lung cancer metastatic:0.43), (AMOXICILLIN:Non-small cell lung cancer metastatic:0.35), (CARFILZOMIB:Non-small cell lung cancer metastatic:0.09), (CEPHALEXIN:Non-small cell lung cancer metastatic:0.45), (CERTOLIZUMAB PEGOL:Non-small cell lung cancer metastatic:0.42), (CHOLESTYRAMINE:Non-small cell lung cancer metastatic:0.01), (CLINDAMYCINCLINDAMYCIN PHOSPHATE:Non-small cell lung cancer metastatic:0.73), (COLCHICINE:Non-small cell lung cancer metastatic:0.18), (DASATINIB:Non-small cell lung cancer metastatic:0.19), (DEXLANSOPRAZOLE:Non-small cell lung cancer metastatic:0.95), (DICYCLOMINE HYDROCHLORIDE:Non-small cell lung cancer metastatic:0.09), (DUTASTERIDE:Non-small cell lung cancer metastatic:0.23), (EPINEPHRINE:Non-small cell lung cancer metastatic:0.37), (ESZOPICLONE:Non-small cell lung cancer metastatic:0.18), (EVEROLIMUS:Non-small cell lung cancer metastatic:0.59), (EVOLOCUMAB:Non-small cell lung cancer metastatic:0.1), (FLUTICASONE FUROATEVILANTEROL TRIFENATATE:Non-small cell lung cancer metastatic:0.18), (IBRUTINIB:Non-small cell lung cancer metastatic:1.11), (ISOSORBIDE MONONITRATE:Non-small cell lung cancer metastatic:0.29), (LENALIDOMIDE:Non-small cell lung cancer metastatic:0.29), (LOVASTATIN:Non-small cell lung cancer metastatic:0.64), (METAXALONE:Non-small cell lung cancer metastatic:0.03), (NILOTINIB:Non-small cell lung cancer metastatic:0.3), (NITROFURANTOIN:Non-small cell lung cancer metastatic:0.08), (OXCARBAZEPINE:Non-small cell lung cancer metastatic:0.14), (PANTOPRAZOLE MAGNESIUM:Non-small cell lung cancer metastatic:0.08), (PAZOPANIB HYDROCHLORIDE:Non-small cell lung cancer metastatic:0.18), (PITAVASTATIN CALCIUM:Non-small cell lung cancer metastatic:0.06), (PRIMIDONE:Non-small cell lung cancer metastatic:0.02), (RANOLAZINE:Non-small cell lung cancer metastatic:0.07), (REGORAFENIB:Non-small cell lung cancer metastatic:0.32), (TOPIRAMATE:Non-small cell lung cancer metastatic:0.33), (VITAMIN B:Non-small cell lung cancer metastatic:0.04), (ACARBOSE:Non-small cell lung cancer stage IV:0.01), (ACETAMINOPHENBUTALBITALCAFFEINE:Non-small cell lung cancer stage IV:0.04), (ADO-TRASTUZUMAB EMTANSINE:Non-small cell lung cancer stage IV:0.22), (ANASTROZOLE:Non-small cell lung cancer stage IV:0.9), (BENAZEPRIL HYDROCHLORIDE:Non-small cell lung cancer stage IV:0.33), (BENAZEPRILBENAZEPRIL HYDROCHLORIDE:Non-small cell lung cancer stage IV:0.08), (BUDESONIDEFORMOTEROL FUMARATE:Non-small cell lung cancer stage IV:0.03), (BUSPIRONE HYDROCHLORIDE:Non-small cell lung cancer stage IV:0.12), (CAPTOPRIL:Non-small cell lung cancer stage IV:0.08), (CARFILZOMIB:Non-small cell lung cancer stage IV:0.07), (CARVEDILOL PHOSPHATE:Non-small cell lung cancer stage IV:0.13), (CEFAZOLIN SODIUM:Non-small cell lung cancer stage IV:0.04), (CEFOTAXIME SODIUM:Non-small cell lung cancer stage IV:0.25), (CERTOLIZUMAB PEGOL:Non-small cell lung cancer stage IV:0.92), (CICLESONIDE:Non-small cell lung cancer stage IV:0.02), (CLOTRIMAZOLE:Non-small cell lung cancer stage IV:0.29), (CLOZAPINE:Non-small cell lung cancer stage IV:0.11), (CORTISONEHYDROCORTISONE:Non-small cell lung cancer stage IV:0.45), (DALTEPARIN:Non-small cell lung cancer stage IV:0.25), (DASATINIB:Non-small cell lung cancer stage IV:0.34), (DEXLANSOPRAZOLE:Non-small cell lung cancer stage IV:0.82), (DIVALPROEX SODIUM:Non-small cell lung cancer stage IV:0.3), (DOCUSATE:Non-small cell lung cancer stage IV:0.69), (ELETRIPTAN HYDROBROMIDE:Non-small cell lung cancer stage IV:0.04), (ERYTHROMYCIN:Non-small cell lung cancer stage IV:0.06), (EVEROLIMUS:Non-small cell lung cancer stage IV:1.02), (EVOLOCUMAB:Non-small cell lung cancer stage IV:0.1), (EZETIMIBESIMVASTATIN:Non-small cell lung cancer stage IV:0.04), (FESOTERODINE FUMARATE:Non-small cell lung cancer stage IV:0.01), (FLECAINIDE:Non-small cell lung cancer stage IV:0.76), (FLUOXETINEFLUOXETINE HYDROCHLORIDE:Non-small cell lung cancer stage IV:0.14), (FORMOTEROL FUMARATE DIHYDRATE\MOMETASONE FUROATE:Non-small cell lung cancer stage IV:0.05), (GLYBURIDE:Non-small cell lung cancer stage IV:0.09), (HEPARIN CALCIUM:Non-small cell lung cancer stage IV:0), (HYDRALAZINE HYDROCHLORIDE:Non-small cell lung cancer stage IV:0.37), (HYDROCHLOROTHIAZIDELISINOPRIL:Non-small cell lung cancer stage IV:0.28), (HYDROXYZINE PAMOATE:Non-small cell lung cancer stage IV:0.29), (IBRUTINIB:Non-small cell lung cancer stage IV:0.65), (ILOPROST:Non-small cell lung cancer stage IV:0.22), (INDAPAMIDEPERINDOPRIL:Non-small cell lung cancer stage IV:0.79), (IRINOTECAN HYDROCHLORIDE:Non-small cell lung cancer stage IV:0.58), (ISOSORBIDE:Non-small cell lung cancer stage IV:0.44), (ISOSORBIDE MONONITRATE:Non-small cell lung cancer stage IV:0.8), (LABETALOLLABETALOL HYDROCHLORIDE:Non-small cell lung cancer stage IV:0.54), (LEDIPASVIRSOFOSBUVIR:Non-small cell lung cancer stage IV:0.12), (LERCANIDIPINE HYDROCHLORIDE:Non-small cell lung cancer stage IV:0.39), (LEUCOVORIN CALCIUM:Non-small cell lung cancer stage IV:0.63), (LEVOLEUCOVORIN CALCIUM:Non-small cell lung cancer stage IV:0.19), (MOMETASONE FUROATE:Non-small cell lung cancer stage IV:0.07), (NILOTINIB:Non-small cell lung cancer stage IV:0.71), (OXCARBAZEPINE:Non-small cell lung cancer stage IV:0.51), (PANTOPRAZOLE MAGNESIUM:Non-small cell lung cancer stage IV:0.82), (PAROXETINEPAROXETINE HYDROCHLORIDE:Non-small cell lung cancer stage IV:0.19), (PAZOPANIB HYDROCHLORIDE:Non-small cell lung cancer stage IV:0.12), (PENICILLIN:Non-small cell lung cancer stage IV:0.3), (PONATINIB:Non-small cell lung cancer stage IV:0.01), (PRAMIPEXOLE:Non-small cell lung cancer stage IV:0.02), (PRAMIPEXOLE DIHYDROCHLORIDE:Non-small cell lung cancer stage IV:0.29), (PREDNISOLONEPREDNISOLONE ACETATEPREDNISOLONE SODIUM SUCCINATE:Non-small cell lung cancer stage IV:0.38), (PRIMIDONE:Non-small cell lung cancer stage IV:0.49), (QUETIAPINE:Non-small cell lung cancer stage IV:0.57), (RANOLAZINE:Non-small cell lung cancer stage IV:0.55), (REGORAFENIB:Non-small cell lung cancer stage IV:0.72), (ROFLUMILAST:Non-small cell lung cancer stage IV:0.26), (SULFAMETHOXAZOLE:Non-small cell lung cancer stage IV:0.38), (TICAGRELOR:Non-small cell lung cancer stage IV:0.02), (TIMOLOL:Non-small cell lung cancer stage IV:0.17), (TIPIRACIL HYDROCHLORIDETRIFLURIDINE:Non-small cell lung cancer stage IV:0.02), (TIZANIDINE:Non-small cell lung cancer stage IV:0.04), (TIZANIDINE HYDROCHLORIDE:Non-small cell lung cancer stage IV:0.19), (TOPIRAMATE:Non-small cell lung cancer stage IV:1.16), (VALGANCICLOVIR HYDROCHLORIDE:Non-small cell lung cancer stage IV:0.14), (VEMURAFENIB:Non-small cell lung cancer stage IV:0), (VITAMIN B:Non-small cell lung cancer stage IV:0.13), (BUDESONIDE:Nuclear magnetic resonance imaging:0.45), (BUMETANIDE:Nuclear magnetic resonance imaging:0.04), (CALCIUM CARBONATECHOLECALCIFEROL:Nuclear magnetic resonance imaging:0.17), (CITALOPRAM HYDROBROMIDE:Nuclear magnetic resonance imaging:0.68), (DIVALPROEX SODIUM:Nuclear magnetic resonance imaging:0.67), (ENOXAPARIN:Nuclear magnetic resonance imaging:0.07), (ESZOPICLONE:Nuclear magnetic resonance imaging:0.05), (EVEROLIMUS:Nuclear magnetic resonance imaging:0.09), (EVOLOCUMAB:Nuclear magnetic resonance imaging:0.33), (IBRUTINIB:Nuclear magnetic resonance imaging:0.04), (LOVASTATIN:Nuclear magnetic resonance imaging:0.45), (NILOTINIB:Nuclear magnetic resonance imaging:0), (PREGABALIN:Nuclear magnetic resonance imaging:0.26), (RANOLAZINE:Nuclear magnetic resonance imaging:0.07), (SACUBITRILVALSARTAN:Nuclear magnetic resonance imaging:0.41), (TERIFLUNOMIDE:Nuclear magnetic resonance imaging:0.71), (TIZANIDINE HYDROCHLORIDE:Nuclear magnetic resonance imaging:0.04), (TORSEMIDE:Nuclear magnetic resonance imaging:0.15), (TRIAMCINOLONE ACETONIDE:Nuclear magnetic resonance imaging:0.08), (VERAPAMIL HYDROCHLORIDE:Nuclear magnetic resonance imaging:0.12), (GADOLINIUM:Nuclear magnetic resonance imaging abdominal:0.11), (.ALPHA.1-PROTEINASE INHIBITOR HUMAN:Nutritional supplementation:1.74), (ACEBUTOLOL HYDROCHLORIDE:Nutritional supplementation:0), (ACENOCOUMAROL:Nutritional supplementation:1.04), (ACETAMINOPHEN\CAFFEINE:Nutritional supplementation:0.62), (ACETAMINOPHENOXYCODONE HYDROCHLORIDEOXYCODONE TEREPHTHALATE:Nutritional supplementation:1.34), (ACTIVATED CHARCOAL:Nutritional supplementation:1.26), (ALBIGLUTIDE:Nutritional supplementation:0.27), (ALOGLIPTIN BENZOATE:Nutritional supplementation:0.38), (AMIKACIN SULFATE:Nutritional supplementation:0.33), (AMISULPRIDE:Nutritional supplementation:0.9), (AMLODIPINEPERINDOPRIL:Nutritional supplementation:0.64), (AMMONIUM LACTATE:Nutritional supplementation:1.37), (AMOXICILLINCLARITHROMYCINESOMEPRAZOLE:Nutritional supplementation:1.07), (ANAGRELIDE HYDROCHLORIDE:Nutritional supplementation:0.43), (ANIDULAFUNGIN:Nutritional supplementation:0.04), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Nutritional supplementation:0.49), (ASCORBIC ACIDBIOTINCYANOCOBALAMINFOLIC ACIDNIACINPANTOTHENIC ACIDPYRIDOXINE\RIBOFLAVINTHIAMINE:Nutritional supplementation:0.21), (ASPIRINDIHYDROXYALUMINUM AMINOACETATE ANHYDROUSMAGNESIUM CARBONATE:Nutritional supplementation:0.45), (ASPIRINMAGNESIUM OXIDE:Nutritional supplementation:0.58), (ATRACURIUM:Nutritional supplementation:0.2), (ATRACURIUM BESYLATE:Nutritional supplementation:0.11), (BENIDIPINE HYDROCHLORIDE:Nutritional supplementation:0.09), (BENZOCAINE:Nutritional supplementation:0.12), (BETAMETHASONE DIPROPIONATECALCIPOTRIENECALCIPOTRIENE HYDRATE:Nutritional supplementation:1.05), (BETAMETHASONE SODIUM PHOSPHATE:Nutritional supplementation:0.27), (BETAMETHASONECALCIPOTRIENE:Nutritional supplementation:0.89), (BETAXOLOL HYDROCHLORIDE:Nutritional supplementation:0.15), (BETHANECHOL CHLORIDE:Nutritional supplementation:0.73), (BLESELUMAB:Nutritional supplementation:0.38), (BLINATUMOMAB:Nutritional supplementation:0.82), (BOSUTINIB:Nutritional supplementation:0.08), (BOTULINUM TOXIN TYPE A:Nutritional supplementation:0.28), (BRIVARACETAM:Nutritional supplementation:0.58), (BUPIVACAINE HYDROCHLORIDE:Nutritional supplementation:0.64), (CABOZANTINIB:Nutritional supplementation:0.15), (CALCITONIN:Nutritional supplementation:1.32), (CALCIUM PHOSPHATE, DIBASIC, ANHYDROUSFERROUS SUCCINATEMAGNESIUM HYDROXIDE:Nutritional supplementation:0.39), (CALCIUM POLYSTYRENE SULFONATE:Nutritional supplementation:0.02), (CAMPHORDIPHENHYDRAMINE\ZINC OXIDE:Nutritional supplementation:0.14), (CEFOTAXIMECEFOTAXIME SODIUM:Nutritional supplementation:0.31), (CEFOXITINCEFOXITIN SODIUM:Nutritional supplementation:0.04), (CEFPROZIL:Nutritional supplementation:0.52), (CERIVASTATIN SODIUM:Nutritional supplementation:0.39), (CERTOPARIN SODIUM:Nutritional supplementation:0.06), (CHLOROTHIAZIDE:Nutritional supplementation:1.12), (CIPROFIBRATE:Nutritional supplementation:0.05), (CLIKSTAR:Nutritional supplementation:0.23), (COBICISTATELVITEGRAVIREMTRICITABINETENOFOVIR DISOPROXIL FUMARATE:Nutritional supplementation:0.04), (CORTICOTROPIN:Nutritional supplementation:1.46), (CORTISONE ACETATE:Nutritional supplementation:0.14), (CYANOCOBALAMINFOLIC ACIDPYRIDOXINE:Nutritional supplementation:0.04), (DABRAFENIB:Nutritional supplementation:0.83), (DABRAFENIB MESYLATE:Nutritional supplementation:0.56), (DACLATASVIR:Nutritional supplementation:0.59), (DEFERIPRONE:Nutritional supplementation:0.79), (DEGARELIX:Nutritional supplementation:0.24), (DEGARELIX ACETATE:Nutritional supplementation:0.16), (DESLANOSIDE:Nutritional supplementation:0.44), (DESOGESTRELETHINYL ESTRADIOL:Nutritional supplementation:0.38), (DESVENLAFAXINE:Nutritional supplementation:0.01), (DEXAMETHASONETOBRAMYCIN:Nutritional supplementation:0.13), (DIGITOXIN:Nutritional supplementation:1.01), (DIPYRONEPROMETHAZINE HYDROCHLORIDE:Nutritional supplementation:0.35), (DISOPYRAMIDE:Nutritional supplementation:0.09), (DOLASETRON MESYLATE:Nutritional supplementation:0.82), (DOLUTEGRAVIR:Nutritional supplementation:0.03), (DORZOLAMIDE HYDROCHLORIDE:Nutritional supplementation:0.48), (DOXEPIN HYDROCHLORIDE:Nutritional supplementation:1.36), (DOXYLAMINE SUCCINATEPYRIDOXINE HYDROCHLORIDE:Nutritional supplementation:0.4), (DROXIDOPA:Nutritional supplementation:0.3), (ECALLANTIDE:Nutritional supplementation:0.15), (ELBASVIRGRAZOPREVIR:Nutritional supplementation:0.47), (ELOTUZUMAB:Nutritional supplementation:1.63), (ELTROMBOPAG OLAMINE:Nutritional supplementation:0.8), (EMPAGLIFLOZINLINAGLIPTIN:Nutritional supplementation:1.63), (EMTRICITABINERILPIVIRINE HYDROCHLORIDETENOFOVIR DISOPROXIL FUMARATE:Nutritional supplementation:0.01), (ENALAPRIL MALEATELERCANIDIPINE HYDROCHLORIDE:Nutritional supplementation:0.32), (EPINASTINE HYDROCHLORIDE:Nutritional supplementation:1.15), (EPIRUBICIN:Nutritional supplementation:1.16), (EPIRUBICIN HYDROCHLORIDE:Nutritional supplementation:0.2), (ERLOTINIB HYDROCHLORIDE:Nutritional supplementation:0.05), (ERTAPENEM SODIUM:Nutritional supplementation:0.2), (ESTRADIOLNORETHINDRONE ACETATE:Nutritional supplementation:0.23), (ESTRIOL:Nutritional supplementation:0.08), (ETIMICIN SULFATE:Nutritional supplementation:0.97), (ETRETINATE:Nutritional supplementation:0.51), (FENOTEROL HYDROBROMIDE:Nutritional supplementation:0.56), (FERRIC HYDROXIDE:Nutritional supplementation:0.29), (FERROUS BISGLYCINATE:Nutritional supplementation:0.14), (FERROUS SULFATEGLYCINE:Nutritional supplementation:0.69), (FIBRINOGEN HUMAN:Nutritional supplementation:1.3), (FLUINDIONE:Nutritional supplementation:1.21), (FLUOXETINE HYDROCHLORIDEOLANZAPINE:Nutritional supplementation:0.15), (FLURAZEPAMFLURAZEPAM HYDROCHLORIDE:Nutritional supplementation:0.18), (FLURBIPROFEN:Nutritional supplementation:0.15), (FLUTAMIDE:Nutritional supplementation:0.2), (FLUTICASONE FUROATESALMETEROL XINAFOATE:Nutritional supplementation:1.94), (FORMOTEROL FUMARATE:Nutritional supplementation:1.61), (FORMOTEROL FUMARATE\MOMETASONE FUROATE:Nutritional supplementation:1.29), (GLYCOPYRROLATEINDACATEROL MALEATE:Nutritional supplementation:0.42), (GLYCOPYRRONIUM:Nutritional supplementation:0.96), (GLYCOPYRRONIUMlNDACATEROL:Nutritional supplementation:1.6), (GUANFACINE:Nutritional supplementation:1.22), (HEPARIN CALCIUM:Nutritional supplementation:0.49), (HUMAN C1-ESTERASE INHIBITOR:Nutritional supplementation:0.07), (HYDROCHLOROTHIAZIDERAMIPRIL:Nutritional supplementation:0.03), (HYOSCYAMINE SULFATEMETHENAMINEMETHYLENE BLUEPHENYL SALICYLATE\SODIUM PHOSPHATE, MONOBASIC, MONOHYDRATE:Nutritional supplementation:0.28), (IBRITUMOMAB TIUXETAN:Nutritional supplementation:0.32), (IDARUCIZUMAB:Nutritional supplementation:0.17), (ILOPROST:Nutritional supplementation:1.26), (IMIPRAMINE HYDROCHLORIDE:Nutritional supplementation:1.49), (IMMUNE GLOBULIN NOS:Nutritional supplementation:0.95), (INDAPAMIDEPERINDOPRIL:Nutritional supplementation:1.53), (INFLIXIMAB-DYYB:Nutritional supplementation:0.1), (INSULIN PORKINSULIN PURIFIED PORK:Nutritional supplementation:0.2), (INTERFERON:Nutritional supplementation:1.84), (INTERFERON ALFA-2A:Nutritional supplementation:1.12), (INTERFERON GAMMA-1B:Nutritional supplementation:0.91), (IRINOTECAN:Nutritional supplementation:0.73), (IRON POLYMALTOSE:Nutritional supplementation:0.1), (ISOFLURANE:Nutritional supplementation:0.4), (IXEKIZUMAB:Nutritional supplementation:0.05), (LANREOTIDE:Nutritional supplementation:0.56), (LENVATINIB:Nutritional supplementation:0.74), (LOPINAVIR:Nutritional supplementation:0.34), (LUTETIUM LU-177:Nutritional supplementation:0.44), (MAGNESIUM ASPARTATE\POTASSIUM ASPARTATE:Nutritional supplementation:0.02), (MEDROXYPROGESTERONE:Nutritional supplementation:0.86), (METFORMIN HYDROCHLORIDESAXAGLIPTIN:Nutritional supplementation:0.2), (METHENAMINE:Nutritional supplementation:0.21), (METHENOLONE ENANTHATE:Nutritional supplementation:1.2), (METHOXY POLYETHYLENE GLYCOL-EPOETIN BETA:Nutritional supplementation:0.66), (METILDIGOXIN:Nutritional supplementation:0.64), (MEXILETINE:Nutritional supplementation:0.26), (MIGLITOL:Nutritional supplementation:0.06), (MIGLUSTAT:Nutritional supplementation:0.56), (MOEXIPRIL HYDROCHLORIDE:Nutritional supplementation:1.08), (MOXONIDINE:Nutritional supplementation:0.71), (NALOXONE:Nutritional supplementation:1.12), (NALOXONE HYDROCHLORIDETILIDINE HYDROCHLORIDE:Nutritional supplementation:1.07), (NARATRIPTAN HYDROCHLORIDE:Nutritional supplementation:0.62), (NEFAZODONE HYDROCHLORIDE:Nutritional supplementation:0.03), (NEOSTIGMINE:Nutritional supplementation:0.76), (ORPHENADRINE:Nutritional supplementation:0.69), (OSELTAMIVIR:Nutritional supplementation:0.89), (OXYMORPHONE:Nutritional supplementation:0.17), (PAPAYA:Nutritional supplementation:0.1), (PATIROMER:Nutritional supplementation:1.21), (PEGZEREPOETIN ALFA:Nutritional supplementation:0.31), (PENICILLIN G:Nutritional supplementation:1.1), (PENICILLIN V:Nutritional supplementation:1.13), (PENTAMIDINE ISETHIONATE:Nutritional supplementation:0.88), (PENTAZOCINE LACTATE:Nutritional supplementation:0.02), (PENTOSTATIN:Nutritional supplementation:0.57), (PHENTOLAMINE:Nutritional supplementation:0.13), (PILOCARPINE:Nutritional supplementation:0.98), (PINDOLOL:Nutritional supplementation:0.02), (PNEUMOCOCCAL VACCINE, POLYVALENT 23:Nutritional supplementation:0.81), (POLYETHYLENE GLYCOL 3350POTASSIUM CHLORIDE\SODIUM BICARBONATE\SODIUM CHLORIDE\SODIUM SULFATE ANHYDROUS:Nutritional supplementation:0.57), (POLYMYXIN B SULFATETRIMETHOPRIM SULFATE:Nutritional supplementation:0.18), (PORFIMER SODIUM:Nutritional supplementation:0.56), (POTASSIUM CITRATETRISODIUM CITRATE DIHYDRATE:Nutritional supplementation:0.48), (PRANLUKAST:Nutritional supplementation:0.48), (PREDNISOLONE SODIUM PHOSPHATE:Nutritional supplementation:0.6), (PRISTINAMYCIN:Nutritional supplementation:0.04), (PROTRIPTYLINE HYDROCHLORIDE:Nutritional supplementation:0.67), (QUINACRINE:Nutritional supplementation:0.7), (RADIATION THERAPY:Nutritional supplementation:1.72), (RILMENIDINE PHOSPHATE:Nutritional supplementation:0.14), (RILPIVIRINE HYDROCHLORIDE:Nutritional supplementation:0.26), (RIZATRIPTAN:Nutritional supplementation:0.86), (ROFECOXIB:Nutritional supplementation:0.27), (RUFINAMIDE:Nutritional supplementation:0.63), (SACUBITRILVALSARTAN:Nutritional supplementation:0.83), (SARPOGRELATE:Nutritional supplementation:0.58), (SELEXIPAG:Nutritional supplementation:0.16), (SEVELAMER:Nutritional supplementation:0.57), (SIBUTRAMINE HYDROCHLORIDE:Nutritional supplementation:0), (SIPULEUCEL-T:Nutritional supplementation:0.98), (SODIUM CITRATE:Nutritional supplementation:0.75), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Nutritional supplementation:0.21), (SUCCINYLCHOLINE CHLORIDE:Nutritional supplementation:0.45), (SUFENTANIL:Nutritional supplementation:0.5), (SULBACTAM SODIUM:Nutritional supplementation:0.69), (TAMOXIFEN CITRATE:Nutritional supplementation:0.8), (TASIMELTEON:Nutritional supplementation:0.27), (TERBUTALINE:Nutritional supplementation:1.07), (TERCONAZOLE:Nutritional supplementation:0.05), (THEOPHYLLINE:Nutritional supplementation:0.88), (THIOCTIC ACID:Nutritional supplementation:0.03), (THIOPENTAL:Nutritional supplementation:0.62), (THIOPENTAL SODIUM:Nutritional supplementation:0.16), (THROMBOPOIETIN:Nutritional supplementation:0.08), (TIPIRACIL HYDROCHLORIDETRIFLURIDINE:Nutritional supplementation:0.69), (TRABECTEDIN:Nutritional supplementation:0.31), (TRAMETINIB:Nutritional supplementation:0.71), (TRAMETINIB DIMETHYL SULFOXIDE:Nutritional supplementation:0.63), (TRANDOLAPRIL:Nutritional supplementation:0.81), (TRIMIPRAMINE MALEATE:Nutritional supplementation:0.34), (TRIPTORELIN PAMOATE:Nutritional supplementation:0.41), (TROSPIUM:Nutritional supplementation:0.08), (UMECLIDINIUM BROMIDE:Nutritional supplementation:1.03), (URAPIDIL:Nutritional supplementation:0.95), (UROKINASE:Nutritional supplementation:0.52), (VALGANCICLOVIR:Nutritional supplementation:0.33), (VANDETANIB:Nutritional supplementation:0.19), (VARDENAFIL HYDROCHLORIDE TRIHYDRATE:Nutritional supplementation:0.6), (VECURONIUM BROMIDE:Nutritional supplementation:0.8), (VINBLASTINE:Nutritional supplementation:0.11), (VINORELBINE TARTRATE:Nutritional supplementation:1.11), (VOGLIBOSE:Nutritional supplementation:0.56), (ZICONOTIDE ACETATE:Nutritional supplementation:0.53), (ZILEUTON:Nutritional supplementation:0.73), (ZIV-AFLIBERCEPT:Nutritional supplementation:0.27), (ACARBOSE:Obesity:0.3), (ACETAMINOPHENBUTALBITALCAFF EINE:Obesity:0.36), (AMIODARONE HYDROCHLORIDE:Obesity:0.85), (AMISULPRIDE:Obesity:0.05), (ATROPINE SULFATEDIPHENOXYLATE HYDROCHLORIDE:Obesity:0.27), (BISACODYL OR DOCUSATE SODIUM:Obesity:0.11), (BUDESONIDEFORMOTEROL FUMARATE:Obesity:0.14), (BUPRENORPHINE HYDROCHLORIDE:Obesity:0.47), (CALCIUMVITAMIN D:Obesity:0.23), (CANDESARTAN CILEXETILHYDROCHLOROTHIAZIDE:Obesity:0.5), (CERTOLIZUMAB PEGOL:Obesity:1), (CLINDAMYCINCLINDAMYCIN PHOSPHATE:Obesity:0.31), (CODEINE:Obesity:0), (DALFAMPRIDINE:Obesity:0.84), (DESL ORATADINE:Obesity:0.28), (DOFETILIDE:Obesity:0.44), (DOXAZOSIN MESYLATE:Obesity:0.08), (DOXEPIN HYDROCHLORIDE:Obesity:0.06), (DUTASTERIDE:Obesity:0.23), (ELETRIPTAN HYDROBROMIDE:Obesity:0.64), (EMPAGLIFLOZINLINAGLIPTIN:Obesity:0.9), (ENALAP RIL MALEATE:Obesity:0.44), (EVEROLIMUS:Obesity:0.42), (FERROUS SULFATEFERROUS SULFATE, DRIED:Obesity:1.44), (FINGOLIMOD HYDROCHLORIDE:Obesity:0.19), (IBRUTINIB:Obesity:1.28), (IMIPRAMINE HYDROCHLORIDE:Obesity:0.1), (LERCANIDIPINE HYDROCHLORIDE:Obesity:0.22), (LOMITAPIDE MESYLATE:Obesity:0.03), (MEDROXYPROGESTERONE ACETATE:Obesity:0.02), (METHYLPREDNISOLONE SODIUM SUCCINATE:Obesity:0.25), (METRONIDAZOLEMETRONIDAZOLE HYDROCHLORIDE:Obesity:0.06), (NADOLOL:Obesity:0.09), (NITROFURANTOIN:Obesity: 0.03), (OXYCODONE HYDROCHLORIDE:Obesity:0.15), (PANTOPRAZOLE MAGNESIUM:Obesity:0.35), (PAZOPANIB HYDROCHLORIDE:Obesity:0.4), (PEGINTERFERON BETA-1A:Obesity:0.08), (PENICILLIN:Obesity:0.1), (PHENOBARBITAL:Obesity:0.11), (POLYETHY LENE GLYCOLS:Obesity:0.38), (RABEPRAZOLE:Obesity:0), (RALOXIFENE HYDROCHLORIDE:Obesity:0.45), (RANOLAZINE:Obesity:1.51), (ROFLUMILAST:Obesity:00.19), (SELEXIPAG:Obesity:0.05), (SUMATRIPTAN:Obesity:0.31), (SUMATRIPTANSUMATR IPTAN SUCCINATE:Obesity:0.84), (TETRACYCLINE:Obesity:0.58), (TIMOLOL:Obesity:0.21), (UME CLIDINIUM BROMIDEVILANTEROL TRIFENATATE:Obesity:0.64), (VITAMIN B:Obesity:0.58), (ZINC:Obesity:0.13), (ACETAMINOPHENOXYCODONEOXYCODONE HYDROCHLORIDE:Obsessive-compulsive disorder:0.1), (ACETAZOLAMIDE:Obsessive-compulsive disorder:0.11), (AMANTADINE:Obsessive-compulsive disorder:0.31), (AMANTADINE HYDROCHLORIDE:Obsessive-compulsive disorder:0.22), (AMIODARONE HYDROCHLORIDE:Obsessive-compulsive disorder:0.12), (ARIPIPRAZOLE LAUROXIL:Obsessive-compulsive disorder:0.52), (AZITHROMYCIN ANHYDROUS:Obsessive-compulsive disorder:0.43), (BUPRENORPHINE HYDROCHLORIDE:Obsessive-compulsive disorder:1.8), (BUPRENORPHINENALOXONE:Obsessive-compulsive disorder:0.32), (CANDESARTAN:Obsessive-compulsive disorder:0.62), (CANDESARTAN CILEXETILHYDROCHLOROTHIAZIDE:Obsessive-compulsive disorder:0.35), (CLINDAMYCIN:Obsessive-compulsive disorder:0.04), (CLOBAZAM:Obsessive-compulsive disorder:0.37), (CLOTRIMAZOLE:Obsessive-compulsive disorder:0.34), (CODEINE:Obsessive-compulsive disorder:0.13), (CORTISONEHYDROCORTISONE:Obsessive-compulsive disorder:0.16), (DACLIZUMAB:Obsessive-compulsive disorder:0.28), (DAPAGLIFLOZIN PROPANEDIOL:Obsessive-compulsive disorder:0.02), (DEVICE:Obsessive-compulsive disorder:0.13), (DICLOFENAC:Obsessive-compulsive disorder:0.74), (DILTIAZEM HYDROCHLORIDE:Obsessive-compulsive disorder:0.72), (DIMENHYDRINATE:Obsessive-compulsive disorder:0.12), (DOCUSATE CALCIUM:Obsessive-compulsive disorder:0.17), (DOXEPIN HYDROCHLORIDE:Obsessive-compulsive disorder:0.15), (ENALAPRIL MALEATE:Obsessive-compulsive disorder:0.5), (ENOXAPARIN:Obsessive-compulsive disorder:0.8), (EVOLOCUMAB:Obsessive-compulsive disorder:0.82), (FESOTERODINE FUMARATE:Obsessive-compulsive disorder:0.27), (FLUDROCORTISONE ACETATE:Obsessive-compulsive disorder:0.07), (FLUPHENAZINE:Obsessive-compulsive disorder:0.02), (GLIPIZIDE:Obsessive-compulsive disorder:0.7), (HYDROXYCHLOROQUINE DIPHOSPHATE:Obsessive-compulsive disorder:0.03), (IBRUTINIB:Obsessive-compulsive disorder:0.6), (ISOSORBIDE:Obsessive-compulsive disorder:0.21), (ISOSORBIDE MONONITRATE:Obsessive-compulsive disorder:0.13), (LEVALBUTEROL HYDROCHLORIDE:Obsessive-compulsive disorder:0.23), (LIOTHYRONINE SODIUM:Obsessive-compulsive disorder:0.16), (METHENAMINE HIPPURATE:Obsessive-compulsive disorder:0.04), (METHYLPREDNISOLONE SODIUM SUCCINATE:Obsessive-compulsive disorder:1.08), (MOXIFLOXACIN HYDROCHLORIDE:Obsessive-compulsive disorder:0.65), (NADOLOL:Obsessive-compulsive disorder:0.12), (NILOTINIB:Obsessive-compulsive disorder:0.17), (NYSTATIN:Obsessive-compulsive disorder:0.4), (OSELTAMIVIR PHOSPHATE:Obsessive-compulsive disorder:0.1), (OXYMORPHONE:Obsessive-compulsive disorder:0), (PANTOPRAZOLE MAGNESIUM:Obsessive-compulsive disorder:0.28), (PEGINTERFERON BETA-1A:Obsessive-compulsive disorder:0.11), (PENTOSAN POLYSULFATE SODIUM:Obsessive-compulsive disorder:0.1), (PERAMPANEL:Obsessive-compulsive disorder:0.18), (PHENOBARBITAL:Obsessive-compulsive disorder:0.91), (POLYETHYLENE GLYCOLS:Obsessive-compulsive disorder:0.89), (PRAZOSIN HYDROCHLORIDE:Obsessive-compulsive disorder:0.04), (RANOLAZINE:Obsessive-compulsive disorder:0.73), (RISEDRONATE SODIUM:Obsessive-compulsive disorder:0.24), (ROFLUMILAST:Obsessive-compulsive disorder:0.07), (SACUBITRILVALSARTAN:Obsessive-compulsive disorder:0.31), (SECUKINUMAB:Obsessive-compulsive disorder:0.79), (SODIUM CHLORIDE:Obsessive-compulsive disorder:0.2), (SOTALOL:Obsessive-compulsive disorder:0.45), (SULFAMETHOXAZOLE:Obsessive-compulsive disorder:0.06), (TELMISARTAN:Obsessive-compulsive disorder:0.58), (TETRACYCLINE:Obsessive-compulsive disorder:0.54), (THALIDOMIDE:Obsessive-compulsive disorder:0.13), (TIZANIDINE HYDROCHLORIDE:Obsessive-compulsive disorder:1.29), (TOLTERODINE TARTRATE:Obsessive-compulsive disorder:0.07), (UMECLIDINIUM BROMIDEVILANTEROL TRIFENATATE:Obsessive-compulsive disorder:0.56), (AMIODARONE HYDROCHLORIDE:Obstructive airways disorder:0.07), (ANASTROZOLE:Obstructive airways disorder:0.2), (BUDESONIDEFORMOTEROL FUMARATE:Obstructive airways disorder:0.03), (BUSPIRONE HYDROCHLORIDE:Obstructive airways disorder:0.14), (CALCIUM CARBONATECHOLECALCIFEROL:Obstructive airways disorder:0.49), (CANDESARTAN CILEXETIL:Obstructive airways disorder:0.19), (CEPHALEXIN:Obstructive airways disorder:0.26), (CERTOLIZUMAB PEGOL:Obstructive airways disorder:0.31), (CLINDAMYCINCLINDAMYCIN PHOSPHATE:Obstructive airways disorder:0.23), (DASATINIB:Obstructive airways disorder:0.06), (ENALAPRIL:Obstructive airways disorder:0.7), (ENOXAPARIN SODIUM:Obstructive airways disorder:0.09), (EVOLOCUMAB:Obstructive airways disorder:0.65), (FLUTICASONE FUROATE:Obstructive airways disorder:0.05), (FORMOTEROL FUMARATE\MOMETASONE FUROATE:Obstructive airways disorder:0.88), (GLIPIZIDE:Obstructive airways disorder:0.01), (GLYCOPYRRONIUM:Obstructive airways disorder:0.55), (ILOPROST:Obstructive airways disorder:1.05), (LOVASTATIN:Obstructive airways disorder:0.14), (MEPOLIZUMAB:Obstructive airways disorder:0.88), (METAXALONE:Obstructive airways disorder:0.06), (METOPROLOL TARTRATE:Obstructive airways disorder:0.36), (OLODATEROL HYDROCHLORIDETIOTROPIUM BROMIDE MONOHYDRATE:Obstructive airways disorder:0.04), (OLOPATADINE:Obstructive airways disorder:0.02), (PANTOPRAZOLE MAGNESIUM:Obstructive airways disorder:0.82), (PRAVASTATIN SODIUM:Obstructive airways disorder:0.55), (SELEXIPAG:Obstructive airways disorder:0.44), (VARENICLINE TARTRATE:Obstructive airways disorder:0.02), (ZOLPIDEMZOLPIDEM TARTRATE:Obstructive airways disorder:0.2), (AMIODARONE:Ocular hypertension:0), (CALCIUM CARBONATECHOLECALCIFEROL:Ocular hypertension:0.18), (CARVEDILOL:Ocular hypertension:0), (DILTIAZEM:Ocular hypertension:0.09), (EVOLOCUMAB:Ocular hypertension:0.4), (FLUTICASONE PROPIONATE:Ocular hypertension:0.16), (PANTOPRAZOLE:Ocular hypertension:0.2), (RABEPRAZOLE SODIUM:Ocular hypertension:0.07), (.ALPHA.1-PROTEINASE INHIBITOR HUMAN:Oedema:1.07), (ABOBOTULINUMTOXINA:Oedema:0.27), (ACETAMINOPHENC AFFEINEOPIUM:Oedema:0.79), (ACETAMINOPHENDIPHENHYDRAMINE:Oedema:0.57), (ACETAMINOPHENMETHOCARBAMOL:Oedema:0.05), (ACLARUBICIN HYDROCHLORIDE:Oedema:0.08), (ADENINE:Oedema:0.47), (ADENOSINE TRIPHOSPHATE:Oedema:0.08), (ALDESLEUKIN:Oedema:0.62), (ALUMINUM HYDROXIDEMAGNESIUM OXIDEOXETHAZAINE:Oedema:0.2), (AMBROXOLCLENBUTEROL:Oedema:0.69), (AME ZINIUM METILSULFATE:Oedema:1.16), (AMINOSALICYLIC ACID:Oedema:0.1), (AMISULPRIDE:Oedema:1.16), (AMLODIPINE BESYLATEHYDROCHLOROTHIAZIDEOLMESARTAN MEDOXOMIL:Oedema:0.6), (AMLODIPINEPERINDOPRIL:Oedema:0.83), (AMLODIPINE VALSARTAN:Oedema:1.36), (AMMONIUM CHLORIDEDEXTROMETHORPHAN\SODIUM CITRATE:Oedema:0.37), (AMOXAPINE:Oedema:0.36), (ANAGLIPTIN:Oedema:0.43), (ANAG RELIDE:Oedema:0.07), (ANAKINRA:Oedema:0.01), (ANTIHISTAMINES NOS:Oedema:0.66), (ARGATROBAN:Oedema:0.14), (ARIPIPRAZOLE LAUROXIL:Oedema:0.2), (ASCORBIC ACIDBIOTINCYANOCOBALAMINFOLIC ACIDNIACINPANTOTHENIC ACIDPYRIDOXINE\RIBOFLAVINTHIAMINE:Oedema:0.08), (ASPARAGINASE:Oedema:1), (ASPIRINCLOPIDOGREL:Oedema:0.55), (ATRACURIUM BESYLATE:Oedema:0.17), (AVOCADO OILSOYBEAN OIL:Oedema:0.02), (BALSALAZIDE DISODIUM:Oedema:0.54), (BECLOMETHASONE:Oedema:0.92), (BECLOMETHASONE DIPROPIONATEFORMOTEROL:Oedema:0.16), (BERAPROST:Oedema:0.64), (BETAMETH ASONE BUTYRATE PROPIONATE:Oedema:0.32), (BETAMETHASONE SODIUM PHOSPHATE:Oedema:1.06), (BETAMETHASONECALCIPOTRIENE:Oedema:0.94), (BETA MIPRONPANIPENEM:Oedema:0.17), (BETAXOLOL HYDROCHLORIDE:Oedema:0.34), (BETHANECHOL CHLORIDE:Oedema:0.74), (BILASTINE:Oedema:0.73), (BLINATUMOMAB:Oedema:1.74), (B OSUTINIB:Oedema:0.32), (BOTULINUM TOXIN TYPE A:Oedema:0.14), (CABOZANTINIB:Oedema:0.27), (CABOZANTINIB S-MALATE:Oedema:0.37), (CALCIUM CHLORIDEDEXTROSE MONOHYDRATEMAGNESIUM CHLORIDE\SODIUM CHLORIDE\SODIUM LACTATE:Oedema:0.37), (CALCIUM PANTOTHENATECYANOCOBALAMINNIACINAMIDEPYRIDOXINE HYDROCHLORIDE\RIBOFLAVINTHIAMINE MONONITRATE:Oedema:0.01), (CAMPHORMENTHOLMETHYL SALICYLATE:Oedema:0.14), (CANAGLIFLOZINMETFORMIN HYDROCHLORIDE:Oedema:1.9), (CARBIDOPAENTACAPONELEVODOPA:Oedema:0.2), (CARTEOLOL HYDROCHLORIDE:Oedema:0.19), (CEFOTAXIMECEFOTAXIME SODIUM:Oedema:0.96), (CEFPODOXIME PROXETIL:Oedema:1), (CELLULASEDIASTASELIPASE:Oedema:0.2), (CHONDROITIN SULFATE (CHICKEN):Oedema:0.39), (CIFENLINE:Oedema:0.56), (CIPROFIBRATE:Oedema:0.9), (CLA DRIBINE:Oedema:0.34), (CLENBUTEROL HYDROCHLORIDE:Oedema:0.38), (CLIKSTAR:Oedema:0.14), (CLOMIPRAMINE HYDROCHLORIDE:Oedema:0.69), (CLOXAZOLAM:Oedema:0.15), (COBICISTATELVITE GRAVIREMTRICITABINETENOFOVIR ALAFENAMIDE FUMARATE:Oedema:0.2), (COLISTIMETHATE SODIUM:Oedema:0.42), (COLISTIN:Oedema:0.48), (CORTISONE ACETATE:Oedema:0.05), (DABIGATRAN ETEXILATE:Oedema:0.04), (DACLIZUMAB:Oedema:2.07), (DANTROLENE SODIUM:Oedema:0.72), (DAUNORUBICIN HYDROCHLORIDE:Oedema:0.37), (DESMOPRESSIN:Oedema:0.66), (DEXAMETHASONE TOBRAMYCIN:Oedema:0.27), (DICLOXACILLIN:Oedema:0.1), (DIFLUNISAL:Oedema:0.16), (DIPHENIDOL HYDROCHLORIDE:Oedema:0.65), (DISTIGMINE BROMIDE:Oedema:0.37), (DOLASETRON MESYLATE:Oedema:0.92), (ECABET SODIUM:Oedema:0.03), (EDARAVONE:Oedema:0.71), (ELTROMBOPAG OLAMINE:Oedema:0.32), (EMPAGLIFLOZINLINAGLIPTIN:Oedema:1.36), (ENALAPRIL MALEATEHYDROCHLOROTHIAZIDE:Oedema:0.65), (ENALAPRIL MALEATELERCANIDIPINE HYDROCHLORIDE:Oedema:0.39), (EPHEDRINE HYDROCHLORIDE:Oedema:0.04), (ESMOLOL HYDROCHLORIDE:Oedema:0.91), (ESTRIOL:Oedema:0.01), (ESTROGENS:Oedema:0.5), (ET HINYL ESTRADIOLNORGESTREL:Oedema:0.12), (ETHYL ICOSAPENTATEICOSAPENT:Oedema:0.53), (ETOPOSIDE PHOSPHATE:Oedema:0.59), (ETRETINATE:Oedema:1.76), (FENOTEROL HYDROBROMIDE:Oedema:0.81), (FERRIC CARBOXYMALTOSE:Oedema:0.21), (FERRIC HYDROXIDE:Oedema:0.58), (FERRIC OXYHYDROXIDE:Oedema:0.33), (FERROUS GLYCINE SULFATE:Oedema:0.08), (FERROUS SULFATEFOLIC ACID:Oedema:0.29), (FERROUS SULFATEGLYCINE:Oedema:0.94), (FEXOFENADINEPSEUDOEPHEDRINE:Oedema:0.75), (FLUOROMETHOLONE:Oedema:0.5), (FLURBIPROFEN:Oedema:0.37), (FORMOTEROL FUMARATE\MOMETASONE FUROATE:Oedema:0.68), (FOSFLUCONAZOLE:Oedema:0.22), (FOSFOMYCIN:Oedema:0.73), (FURSULTIAMINE:Oedema:0.41), (FUSIDIC ACID:Oedema:1.91), (GABEXATE MESYLATE:Oedema:0.04), (GEFITINIB:Oedema:0.95), (GLIPIZIDEMETFORMIN HYDROCHLORIDE:Oedema:0.24), (GLYBURIDEMETFORMIN HYDROCHLORIDE:Oedema:0.25), (GLYCOPYRRONIUM:Oedema:0.41), (GUANFACINE:0 edema:0.95), (GUANFACINE HYDROCHLORIDE:Oedema:0.25), (HERBALSSAW PALMETTO:Oedema:1.04), (HYDROCHLOROTHIAZIDELOSARTAN:Oedema:0.16), (HYD ROCODONE BITARTRATEIBUPROFEN:Oedema:0.75), (HYDROXYCHLOROQUINE DIPHOSPHATE:Oedema:1.15), (IBRITUMOMAB TIUXETAN:Oedema:1.64), (ICOSAPENT:Oedema:0.78), (IGURATIMOD:Oedema:0.2), (IMIG LUCERASE:Oedema:0.58), (IMIQUIMOD:Oedema:0.07), (IMMUNE GLOBULIN NOS:Oedema:0.38), (INDACATEROL:Oedema:0.89), (INFLIXIMAB-DYYB:Oedema:0.88), (INTERFERON ALFA-2A:Oedema:1.66), (INTERFERON ALFA-2B:Oedema:1.46), (INTERFERON GAMMA-1B:Oedema:0.5), (ISONIAZIDRIFAMPIN:Oedema:0.35), (IVABRADINE HYDROCHLORIDE:Oedema:0.42), (KETAMINE HYDROCHLORIDE:Oedema:0.51), (LAPATINIB:Oedema:0.15), (LAPATINIB DITOSYLATE:Oedema:1.1), (LECITHIN:Oedema:0.03), (LENVATINIB:Oedema:0.89), (LEVO MILNACIPRAN HYDROCHLORIDE:Oedema:0.06), (LEVOSIMENDAN:Oedema:0.25), (LINSIDOMINE:Oede ma:0.3), (LORNOXICAM:Oedema:0.25), (LUTETIUM LU-177:Oedema:0.31), (LYSOZYME HYDROCHLORIDE:Oedema:0.21), (MEPOLIZUMAB:Oedema:0.44), (METFORMIN HYDROCHLORIDEROSIGLITAZONE MALEATE:Oedema:0.07), (METFORMIN HYDROCHLORIDESAXAGLIPTIN:Oedema:0.19), (METFORMIN HYDROCHLORIDESAXAGLIPTIN HYDROCHLORIDE:Oedema:0.55), (METFORMINSITAGLIPTIN:Oedema:0.12), (METHEN AMINE HIPPURATE:Oedema:2.05), (METHYLENE BLUE:Oedema:0.04), (MEXILETINE:Oedema:0.23), (MITIGLINIDE CALCIUM HYDRATEVOGLIBOSE:Oedema:0.19), (NALOXONE:Oedema:0.99), (NEOMYCIN:Oedema: 0.22), (OLOPATADINE:Oedema:0.47), (OPIPRAMOL:Oedema:0.29), (OXYBUTYNINOXYB UTYNIN CHLORIDE:Oedema:0.4), (PARATHYROID HORMONE:Oedema:0.56), (PARICALCITOL:Oedema:1.18), (PASIREOTIDE:Oedema:0.89), (P EGINTERFERON BETA-1A:Oedema:1.59), (PENICILLIN V:Oedema:1.34), (PENTOSTATIN:Oedema:1.37), (PHENOBARBITAL SODIUM:Oedema:0.08), (PILOCARPINE:Oedema:1), (PIMECROLIMUS:Oedema:0.38), (PIMO BENDAN:Oedema:1.65), (POLYETHYLENE GLYCOL 3350POTASSIUM CHLORIDE\SODIUM BICARBONATE\SODIUM CHLORIDE\SODIUM SULFATE ANHYDROUS:Oedema:0.62), (POLYSTYRENE SULFONIC ACID:Oedema:0.27), (PORFIMER SODIUM:Oedema:0.19), (POTASSIUM IODIDE:Oedema:0.18), (POTASSIUM PHOSPHATE, MONOBASIC:Oedema:0.06), (POVIDONE:Oedema:0.22), (PRASUGREL:Oedema:0.27), (PRE DNISOLONE SODIUM PHOSPHATE:Oedema:0.96), (PROBUCOL:Oedema:0.06), (PROTRIPTYLINE HYDROCHLORIDE:Oedema:0.29), (RADIATION THERAPY:Oedema:1.75), (RETINOL:Oedema:0.69), (RILPIVIRINE HYDROCHLORIDE:Oedema:0.17), (ROPIVACAINE:Oedema:0.25), (RUCAPARIB CAMSYLATE:Oedema:0.26), (SARPOGRELATE HYDROCHLORIDE:Oedema:0.17), (SAW PALMETTO:Oedema:0.53), (SEVOFLURANE:Oedema:1.02), (SODIUM CITRATE:Oedema:0.29), (SODIUM FLUORIDE:Oedema:1.03), (SODIUM GUALENATE MONOHYDRATE:Oedema:0.03), (SOFOSBUVIRVELPATASVIR:Oedema:0.43), (SORBITO L:Oedema:0.43), (SPIRAMYCIN:Oedema:0.05), (SUCCINYLCHOLINE CHLORIDE:Oedema:0.36), (SUPLATAST TOSILATE:Oedema:0.08), (TAZOBACTAM:Oedema:1.2), (TEDUGLUTIDE\WATER:Oedema: 1.44), (TEGAFURURACIL:Oedema:0.05), (TERBUTALINE SULFATE:Oedema:2.23), (TESTOSTERONE UNDECANOATE:Oedema:1.36), (TETRACYCLINE:Oedema:1.54), (THIAMYLAL SODIUM:Oedema:0.12), (TIMOLOL MALEATETRAVOPROST:Oedema:0.66), (TIPEPIDINE HIBENZATE:Oedema:0.2), (TOSUFLOXACIN TOSYLATE:Oedema:0.1), (TRIMIPRAMINE MALEATE:Oedema:1.01), (TRIPTORELIN PAMOATE:Oedema:0.46), (TROSPIUM:Oedema:0.07), (TROXIPIDE:Oedema:0.12), (UMECLI DINIUM BROMIDE:Oedema:0.96), (VINORELBINEVINORELBINE TARTRATE:Oedema:0.33), (VISMODEGIB:Oedema:0.07), (ZILEUTON:Oedema:0.45), (ZONI SAMIDE:Oedema:0.66), (PYRAZINAMIDE:Oedema due to cardiac disease:0.57), (ACETAMINOPHEN\CAFFEINE:Oedema peripheral:0.26), (ACETAMINOPHENOXYCODONE HYDROCHLORIDEOXYCODONE TEREPHTHALATE:Oedema peripheral:0.53), (ACETAMINOPHENPROPOXYPHENE HYDROCHLORIDE:Oedema peripheral:0.28), (ACITRETIN:Oedema peripheral:0.23), (AGALSIDASE BETA:Oedema peripheral:0.8), (ALCOHOL:Oedema peripheral:0.45), (ALFUZOSIN HYDROCHLORIDE:Oedema peripheral:0.6), (AMIKACIN SULFATE:Oedema peripheral:0.52), (AMINO ACIDSDEXTROSEELECTROLYTES NOS:Oedema peripheral:0.47), (AMLODIPINE BESYLATEBENAZEPRIL HYDROCHLORIDE:Oedema peripheral:0.16), (AMLODIPINE BESYLATEHYDROCHLOROTHIAZIDEVALSARTAN:Oedema peripheral:0.5), (AMLODIPINEVALSARTAN:Oedema peripheral:0.78), (AMPHETAMINE ASPARTATEAMPHETAMINE SULFATEDEXTROAMPHETAMINE SACCHARATEDEXTROAMPHETAMINE SULFATE:Oedema peripheral:1.28), (ANAGRELIDE HYDROCHLORIDE:Oedema peripheral:0.19), (ANTIHISTAMINES NOS:Oedema peripheral:0.26), (ASPIRINMAGNESIUM OXIDE:Oedema peripheral:0.2), (ASUNAPREVIR:Oedema peripheral:0.42), (ATROPINE SULFATE:Oedema peripheral:0.19), (ATROPINEDIPHENOXYLATE:Oedema peripheral:0.04), (BECLOMETHASONE:Oedema peripheral:0.2), (BEPRIDIL:Oedema peripheral:0.45), (BETAMETHASONE DIPROPIONATECALCIPOTRIENE:Oedema peripheral:0.28), (BETAMETHASONE DIPROPIONATECALCIPOTRIENECALCIPOTRIENE HYDRATE:Oedema peripheral:0.1), (BETAMETHASONECALCIPOTRIENE:Oedema peripheral:0.21), (BETHANECHOL CHLORIDE:Oedema peripheral:0.01), (BIMATOPROST:Oedema peripheral:0.35), (BLINATUMOMAB:Oedema peripheral:0.28), (BUDESONIDEFORMOTEROL:Oedema peripheral:0.01), (CABAZITAXEL:Oedema peripheral:0.19), (CABOZANTINIB S-MALATE:Oedema peripheral:0.05), (CALCITONIN:Oedema peripheral:0.76), (CALCITONIN SALMON:Oedema peripheral:0.95), (CALCIUM ACETATE:Oedema peripheral:1.51), (CALCIUM CARBONATEERGOCALCIFEROLRETINOL:Oedema peripheral:0.04), (CALCIUM\MAGNESIUM:Oedema peripheral:0.06), (CANAGLIFLOZINMETFORMIN HYDROCHLORIDE:Oedema peripheral:1.01), (CAPTOPRIL:Oedema peripheral:1.31), (CARBAZOCHROME SODIUM SULFONATE:Oedema peripheral:0.09), (CARBIMAZOLE:Oedema peripheral:0.73), (CARVEDILOL PHOSPHATE:Oedema peripheral:1.67), (CEFOTAXIME SODIUM:Oedema peripheral:1.09), (CEFOTAXIMECEFOTAXIME SODIUM:Oedema peripheral:0.17), (CETUXIMAB:Oedema peripheral:1.47), (CHLORAMBUCIL:Oedema peripheral:0.61), (CHLORDIAZEPOXIDE HYDROCHLORIDECLIDINIUM BROMIDE:Oedema peripheral:0.07), (COD LIVER OIL:Oedema peripheral:0.17), (CYAMEMAZINE:Oedema peripheral:0.15), (CYPROHEPTADINE:Oedema peripheral:0.25), (DABRAFENIB MESYLATE:Oedema peripheral:0.23), (DACLIZUMAB:Oedema peripheral:0.87), (DANTROLENE SODIUM:Oedema peripheral:0.14), (DAPAGLIFLOZIN PROPANEDIOL:Oedema peripheral:0.66), (DAPTOMYCIN:Oedema peripheral:0.04), (DASATINIB:Oedema peripheral:2.59), (DEFLAZACORT:Oedema peripheral:1.26), (DESONIDE:Oedema peripheral:0.45), (DIGITOXIN:Oedema peripheral:1.02), (DOBUTAMINE HYDROCHLORIDE:Oedema peripheral:0.2), (DOLASETRON MESYLATE:Oedema peripheral:0.43), (DORZOLAMIDE:Oedema peripheral:0.29), (DOXEPIN HYDROCHLORIDE:Oedema peripheral:0.66), (DUTASTERIDETAMSULOSIN HYDROCHLORIDE:Oedema peripheral:0.33), (ECONAZOLE:Oedema peripheral:1.34), (ELBASVIRGRAZOPREVIR:Oedema peripheral:0.02), (ELTROMBOPAG OLAMINE:Oedema peripheral:0.36), (EMPAGLIFLOZINLINAGLIPTIN:Oedema peripheral:0.77), (ERIBULIN:Oedema peripheral:0.16), (ESTROGENS, CONJUGATEDMEDROXYPROGESTERONE ACETATE:Oedema peripheral:0.44), (ETHAMBUTOL HYDROCHLORIDE:Oedema peripheral:0.97), (ETRETINATE:Oedema peripheral:0.63), (FERROUS SULFATEGLYCINE:Oedema peripheral:0.31), (FISH OIL\OMEGA-3 FATTY ACIDS:Oedema peripheral:0.23), (FONDAPARINUX SODIUM:Oedema peripheral:0.74), (FORMOTEROL:Oedema peripheral:1.15), (FORMOTEROL FUMARATE\MOMETASONE FUROATE:Oedema peripheral:0.63), (FOSFOMYCIN:Oedema peripheral:0.08), (GEFITINIB:Oedema peripheral:0.38), (GENTAMICIN:Oedema peripheral:0.58), (GLYCOPYRROLATE:Oedema peripheral:0.69), (GUANFACINE:Oedema peripheral:0.01), (HERBALSSAW PALMETTO:Oedema peripheral:0.46), (HYDROCHLOROTHIAZIDEIRBESARTAN:Oedema peripheral:1.83), (HYDROCODONE BITARTRATEIBUPROFEN:Oedema peripheral:0.05), (IBRITUMOMAB TIUXETAN:Oedema peripheral:0.12), (IMIGLUCERASE:Oedema peripheral:0.02), (IMIPRAMINE HYDROCHLORIDE:Oedema peripheral:1.01), (IMMUNE GLOBULIN NOS:Oedema peripheral:0.23), (INFLIXIMAB-DYYB:Oedema peripheral:0.13), (INTERFERON ALFA-2A:Oedema peripheral:0.89), (INTERFERON ALFA-2B:Oedema peripheral:0.82), (IRINOTECAN HYDROCHLORIDE:Oedema peripheral:1.42), (KETOROLACKETOROLAC TROMETHAMINE:Oedema peripheral:0.41), (LACOSAMIDE:Oedema peripheral:0.01), (LACTASE:Oedema peripheral:0.35), (LAFUTIDINE:Oedema peripheral:0.58), (LENVATINIB:Oedema peripheral:0.35), (LEVALBUTEROL:Oedema peripheral:0.29), (LEVOTHYROXINE SODIUMPOTASSIUM IODIDE:Oedema peripheral:0.14), (LINSIDOMINE:Oedema peripheral:0.02), (LIOTHYRONINE SODIUM:Oedema peripheral:0.23), (LITHIUM:Oedema peripheral:0.09), (MEPOLIZUMAB:Oedema peripheral:0.53), (MERCAPTOPURINE:Oedema peripheral:0.1), (METFORMIN HYDROCHLORIDESAXAGLIPTIN HYDROCHLORIDE:Oedema peripheral:0.09), (METFORMIN HYDROCHLORIDEVILDAGLIPTIN:Oedema peripheral:0.92), (METFORMIN PAMOATE:Oedema peripheral:0.15), (METHOXY POLYETHYLENE GLYCOL-EPOETIN BETA:Oedema peripheral:0.01), (MEXILETINE HYDROCHLORIDE:Oedema peripheral:0.39), (MIDODRINE HYDROCHLORIDE:Oedema peripheral:0.27), (NALOXONE:Oedema peripheral:0.06), (NOREPINEPHRINE:Oedema peripheral:0.2), (NOREPINEPHRINE BITARTRATE:Oedema peripheral:0.43), (ORPHENADRINE:Oedema peripheral:0.4), (OXCARBAZEPINE:Oedema peripheral:2.02), (PARICALCITOL:Oedema peripheral:0.2), (PEGINTERFERON BETA-1A:Oedema peripheral:0.91), (PENICILLIN:Oedema peripheral:1.06), (PENTOSTATIN:Oedema peripheral:0.01), (PHENYTOIN SODIUM:Oedema peripheral:0.65), (PHLOROGLUCINOL1,3,5-TRIMETHOXYBENZENE:Oedema peripheral:0.15), (PILOCARPINE:Oedema peripheral:0.25), (PIMECROLIMUS:Oedema peripheral:0.05), (PIMOBENDAN:Oedema peripheral:0.46), (PIROXICAM:Oedema peripheral:0.85), (POLYETHYLENE GLYCOL 3350POTASSIUM CHLORIDE\SODIUM BICARBONATE\SODIUM CHLORIDE\SODIUM SULFATE ANHYDROUS:Oedema peripheral:0.11), (PRANLUKAST:Oedema peripheral:0.3), (PRISTINAMYCIN:Oedema peripheral:0.34), (PROGESTERONE:Oedema peripheral:0.44), (PYRIDOXAL:Oedema peripheral:0.08), (QUINAPRIL:Oedema peripheral:0.38), (RADIATION THERAPY:Oedema peripheral:0.64), (RALTEGRAVIR POTASSIUM:Oedema peripheral:0.54), (REGORAFENIB:Oedema peripheral:1.34), (RETINOL:Oedema peripheral:0.24), (RILMENIDINE PHOSPHATE:Oedema peripheral:0.22), (RIZATRIPTAN:Oedema peripheral:0.02), (SALMETEROL:Oedema peripheral:0.27), (SELEXIPAG:Oedema peripheral:0.67), (SEVELAMER HYDROCHLORIDE:Oedema peripheral:0.51), (SOFOSBUVIRVELPATASVIR:Oedema peripheral:0.01), (TEDUGLUTIDE\WATER:Oedema peripheral:0.14), (TELOTRISTAT ETHYL:Oedema peripheral:0.24), (TESTOSTERONE UNDECANOATE:Oedema peripheral:0.24), (TETRACYCLINE:Oedema peripheral:1.13), (THYROID, PORCINE:Oedema peripheral:0.22), (TINZAPARIN:Oedema peripheral:0.81), (TOLTERODINE:Oedema peripheral:0.28), (TRAMETINIB DIMETHYL SULFOXIDE:Oedema peripheral:0.15), (TRIMIPRAMINE MALEATE:Oedema peripheral:0.13), (UMECLIDINIUM BROMIDE:Oedema peripheral:0.62), (UMECLIDINIUM BROMIDEVILANTEROL TRIFENATATE:Oedema peripheral:0.98), (VOGLIBOSE:Oedema peripheral:0.67), (ZILEUTON:Oedema peripheral:0.1), (ZIPRASIDONE HYDROCHLORIDEZIPRASIDONE MESYLATE:Oedema peripheral:0.05), (ZIV-AFLIBERCEPT:Oedema peripheral:0.21), (DILTIAZEM:Oesophageal candidiasis:0), (ENALAPRIL:Oesophageal candidiasis:0.57), (IBRUTINIB:Oesophageal candidiasis:0.25), (NAPROXEN:Oesophageal candidiasis:0.37), (ADO-TRASTUZUMAB EMTANSINE:Oesophageal carcinoma:0.55), (AMOXICILLIN:Oesophageal carcinoma:0.1), (BUMETANIDE:Oesophageal carcinoma:0.75), (BUPROPION HYDROCHLORIDE:Oesophageal carcinoma:0.31), (CALCIUM CARBONATECHOLECALCIFEROL:Oesophageal carcinoma:0.54), (CALCIUMVITAMIN D:Oesophageal carcinoma:0.29), (CANDESARTAN CILEXETILHYDROCHLOROTHIAZIDE:Oesophageal carcinoma:0.01), (CERTOLIZUMAB PEGOL:Oesophageal carcinoma:0.53), (CHOLESTYRAMINE:Oesophageal carcinoma:0.08), (CLONIDINE:Oesophageal carcinoma:0.27), (CORTISONEHYDROCORTISONE:Oesophageal carcinoma:0.12), (DICLOFENAC:Oesophageal carcinoma:0.36), (DICLOFENAC SODIUM:Oesophageal carcinoma:0.52), (EPINEPHRINE:Oesophageal carcinoma:0.49), (ESZOPICLONE:Oesophageal carcinoma:0.23), (EVOLOCUMAB:Oesophageal carcinoma:0.25), (EXEMESTANE:Oesophageal carcinoma:0.34), (FULVESTRANT:Oesophageal carcinoma:0.02), (ISOSORBIDE:Oesophageal carcinoma:0.04), (LEDIPASVIRSOFOSBUVIR:Oesophageal carcinoma:0.06), (LENALIDOMIDE:Oesophageal carcinoma:0.17), (LINAGLIPTIN:Oesophageal carcinoma:0.24), (MELOXICAM:Oesophageal carcinoma:0.44), (METRONIDAZOLEMETRONIDAZOLE HYDROCHLORIDE:Oesophageal carcinoma:0.01), (NITROFURANTOIN:Oesophageal carcinoma:0.21), (OXCARBAZEPINE:Oesophageal carcinoma:0.61), (OXYGEN:Oesophageal carcinoma:0.38), (PANTOPRAZOLE MAGNESIUM:Oesophageal carcinoma:0.04), (PEMETREXED:Oesophageal carcinoma:0.14), (PRAVASTATIN SODIUM:Oesophageal carcinoma:0.34), (RANOLAZINE:Oesophageal carcinoma:0.16), (REGORAFENIB:Oesophageal carcinoma:0.78), (SACUBITRILVALSARTAN:Oesophageal carcinoma:0.27), (TELMISARTAN:Oesophageal carcinoma:0.26), (TEMAZEPAM:Oesophageal carcinoma:0.03), (TOCILIZUMAB:Oesophageal carcinoma:0.36), (TORSEMIDE:Oesophageal carcinoma:0.4), (VENLAFAXINE HYDROCHLORIDE:Oesophageal carcinoma:0.54), (ARMODAFINIL:Oesophageal disorder:0.06), (BUSPIRONE HYDROCHLORIDE:Oesophageal disorder:0.32), (CALCIUM CARBONATECHOLECALCIFEROL:Oesophageal disorder:0.24), (CERTOLIZUMAB PEGOL:Oesophageal disorder:0.52), (DICLOFENAC SODIUM:Oesophageal disorder:0.19), (DIGOXIN:Oesophageal disorder:0.66), (EVOLOCUMAB:Oesophageal disorder:0.42), (IBRUTINIB:Oesophageal disorder:0.24), (PAROXETINE:Oesophageal disorder:0.01), (PRAVASTATIN SODIUM:Oesophageal disorder:0.16), (SACUBITRILVALSARTAN:Oesophageal disorder:0.15), (TAMSULOSIN:Oesophageal disorder:0.22), (TERIFLUNOMIDE:Oesophageal disorder:0.7), (ARMODAFINIL:Oesophageal spasm:0.15), (BUDESONIDE:Oesophageal spasm:0.41), (CERTOLIZUMAB PEGOL:Oesophageal spasm:0.01), (DICLOFENAC:Oesophageal spasm:0.07), (DIPHENHYDRAMINE:Oesophageal spasm:0.06), (ENALAPRIL:Oesophageal spasm:0.42), (EPINEPHRINE:Oesophageal spasm:0.03), (ESCITALOPRAM OXALATE:Oesophageal spasm:0.59), (IBRUTINIB:Oesophageal spasm:0.19), (IRON:Oesophageal spasm:0.17), (LOVASTATIN:Oesophageal spasm:0.34), (METHYLPREDNISOLONE SODIUM SUCCINATE:Oesophageal spasm:0.06), (METRONIDAZOLE:Oesophageal spasm:0.28), (PAROXETINE:Oesophageal spasm:0.07), (RANOLAZINE:Oesophageal spasm:0.1), (SACUBITRILVALSARTAN:Oesophageal spasm:0.13), (SODIUM OXYBATE:Oesophageal spasm:0.27), (TERIFLUNOMIDE:Oesophageal spasm:0.5), (WARFARIN:Oesophageal spasm:0.55), (ACETAMINOPHENBUTALBITALCAFFEINE:Oesophagitis:0.35), (ALENDRO NIC ACID:Oesophagitis:0.14), (BENAZEPRIL HYDROCHLORIDE:Oesophagitis:0.4), (CALCITRIOL:Oesophagitis:0.61), (CALCIUM ACETATE:Oesophagitis:0.14), (CALCIUM CITRATE:Oesophagitis:0.27), (CANDESARTAN CILEXETILHYDROCHLOROTHIAZIDE:Oesophagitis:0.94), (CEFOTAXIME SODIUM:Oesophagitis:0.03), (CLOTRIMAZOLE:Oesophagitis:0.46), (CYCLOBENZAPRINE HYDROCHLORIDE:Oesophagitis:0.62), (DALFAMPRIDINE:Oesophagitis:0.51), (DALTEPAR IN:Oesophagitis:0.22), (DANAZOL:Oesophagitis:0.01), (DASATINIB:Oesophagitis:1.47), (DEV ICE:Oesophagitis:0.24), (DIGITOXIN:Oesophagitis:0.03), (DIVALPROEX SODIUM:Oesophagitis:0.35), (DOFETILIDE:Oesophagitis:0.08), (DOXAZOSIN MESYLATE:Oesophagitis:0.43), (DOXAZOSINDOXAZOSIN MESYLATE:Oesophagitis:0.51), (DRONABINOL:Oesophagitis:0.13), (ELETRIPTAN HYDROBROMIDE:Oesophagitis:0.26), (EPINEPHRINE:Oesophagitis:0.93), (EXEMESTANE: Oesophagitis:0.16), (EZETIMIBESIMVASTATIN:Oesophagitis:0.44), (FESOTERODINE FUMARATE:Oesophagitis:0.29), (FINGOLIMOD HYDROCHLORIDE:Oesophagitis:0.45), (FLUOXETINEFLUOXETINE HYDROCHLORIDE:Oesophagitis:0.13), (FLUTICASONE FUROATE:Oesophagitis:0.03), (FLUTICASONE FUROATEVILANTEROL TRIFENATATE:Oesophagitis:0.17), (FORMOTEROL FUMARATE:Oesophagitis:0.08), (FORMOTEROL FUMARATE DIHYDRATE\MOMETASONE FUROATE:Oesophagitis:0.08), (FULVESTRANT:Oesophagitis:0.29), (GLYBURIDE:Oesophagi tis:0.21), (GLYCOPYRRONIUMINDACATEROL:Oesophagitis:0.12), (GUAIFENESIN:Oesop hagitis:0.07), (HERBALSTURMERIC:Oesophagitis:0.03), (HYDROCHLOROTHIAZIDELOS ARTAN POTASSIUM:Oesophagitis:0.18), (HYDROCHLOROTHIAZIDETELMISARTAN:Oesophagiti s:0.02), (HYDROXYCHLOROQUINE DIPHOSPHATE:Oesophagitis:0.05), (HYDROXYCHLOROQUINE SULFATE:Oesophagitis:0.68), (HYDROXYZINE PAMOATE:Oesophagitis:0.24), (ILOPROST:Oesophagitis:0.52), (IMATINIB MESYLATE:Oesophagitis:1.35), (INDAPAMIDEPERINDOPRIL:Oesophagitis:0.63), (INDOM ETHACIN:Oesophagitis:0.45), (INFLUENZA VIRUS VACCINE:Oesophagitis:0.3), (IRINOTECAN HYDROCHLORIDE:Oesophagitis:0.27), (ITRACONAZOLE:Oesophagitis:0.07), (LABETALO LLABETALOL HYDROCHLORIDE:Oesophagitis:0.34), (LACOSAMIDE:Oesophagitis:0.11), (LERCANIDIPI NE HYDROCHLORIDE:Oesophagitis:0.78), (LITHIUM CARBONATE:Oesophagitis:0.32), (MEGESTROL ACETATE:Oesophagitis:0.06), (METHYLPREDNISOLONE ACETATE:Oesophagitis:0.34), (MOXIFLOXACIN HYDROCHLORIDE:Oesophagitis:0.95), (NALOXONE HYDROCHLORIDEOXYCODONE HYDROCHLORIDE:Oesophagitis:0.71), (NAPROXEN SODIUM:Oesophagitis:0.31), (NINTEDANIB:Oesophagitis:0.07), (OXYBUTYNIN CHLORIDE:Oesophagitis:0.14), (PAROXETINEPAROXETINE HYDROCHLORIDE:Oesophagitis:0.19), (PAZOPANIB HYDROCHLORIDE:Oesophagitis:0.8), (PEMETREXED:Oesophagitis:0.16), (PEMETREXED DISODIUM:Oesophagitis:0), (PHENYTOIN:Oesophagitis:0.18), (POLYETHYLENE GLYCOLS:Oesophagitis:1.04), (POSACONAZOLE:Oesophagitis:0.07), (PREDNISOLONEPR EDNISOLONE ACETATEPREDNISOLONE SODIUM SUCCINATE:Oesophagitis:0.26), (PRIMIDONE:Oesophagitis:0.85), (QUETIAPINE:Oesophagit is:0.3), (RANOLAZINE:Oesophagitis:0.95), (RIZATRIPTAN BENZOATE:Oesophagitis:0.14), (SACUBITRILVALSARTAN:Oesophagitis:1.11), (SECUKIN UMAB:Oesophagitis:0.52), (SELEXIPAG:Oesophagitis:0.35), (SITAGLIPTIN:Oesophagitis:0.05), (SULFAMETHOXAZOLE:Oesophagitis:0.36), (TETRACYCLINE:Oesophagitis:0.14), (TOLT ERODINE TARTRATE:Oesophagitis:0.27), (TRIAMTERENE:Oesophagitis:0.12), (UMECLIDINIUM BROMIDEVILANTEROL TRIFENATATE:Oesophagitis:0.3), (VINCRISTINE SULFATE:Oesophagitis:1.23), (RISEDRONATE SODIUM:Oestrogen receptor positive breast cancer:0.06), (ANASTROZOLE:Oestrogen replacement therapy:0.44), (CALCIUM CARBONATECHOLECALCIFEROL:Oestrogen replacement therapy:0.27), (CALCIUM CITRATE:Oestrogen replacement therapy:0.35), (DIPHENHYDRAMINE:Oestrogen replacement therapy:0.35), (ENALAPRIL MALEATE:Oestrogen replacement therapy:0.03), (EPINEPHRINE:Oestrogen replacement therapy:0.16), (ESZOPICLONE:Oestrogen replacement therapy:0.65), (ETHINYL ESTRADIOLNORGESTIMATE:Oestrogen replacement therapy:0.01), (EVOLOCUMAB:Oestrogen replacement therapy:0.68), (HYDROCHLOROTHIAZIDELISINOPRIL:Oestrogen replacement therapy:0.03), (HYOSCYAMINE:Oestrogen replacement therapy:0.1), (IBRUTINIB:Oestrogen replacement therapy:0.01), (INDOMETHACIN:Oestrogen replacement therapy:0.13), (LABETALOLLABETALOL HYDROCHLORIDE:Oestrogen replacement therapy:0), (LOVASTATIN:Oestrogen replacement therapy:0.87), (METAXALONE:Oestrogen replacement therapy:0.34), (NITROFURANTOIN:Oestrogen replacement therapy:0.2), (PAROXETINE:Oestrogen replacement therapy:0.04), (PEGINTERFERON BETA-1A:Oestrogen replacement therapy:0.05), (POLYETHYLENE GLYCOLS:Oestrogen replacement therapy:0.09), (RAMIPRIL:Oestrogen replacement therapy:0.34), (RANOLAZINE:Oestrogen replacement therapy:0.43), (SECUKINUMAB:Oestrogen replacement therapy:0.07), (SUMATRIPTAN:Oestrogen replacement therapy:0.07), (SUMATRIPTANSUMATRIPTAN SUCCINATE:Oestrogen replacement therapy:0.43), (TAMSULOSIN:Oestrogen replacement therapy:0.4), (TERIFLUNOMIDE:Oestrogen replacement therapy:0.63), (TIZANIDINE HYDROCHLORIDE:Oestrogen replacement therapy:0.19), (TOCILIZUMAB:Oestrogen replacement therapy:0.04), (UMECLIDINIUM BROMIDEVILANTEROL TRIFENATATE:Oestrogen replacement therapy:0.11), (VITAMIN B:Oestrogen replacement therapy:0.24), (WARFARIN:Oestrogen replacement therapy:0.47), (BICALUTAMIDE:Oestrogen therapy:0.2), (DEXLANSOPRAZOLE:Oestrogen therapy:0.09), (NAPROXEN:Oestrogen therapy:0.11), (ABACAVIR SULFATEDOLUTEGRAVIR SODIUMLAMIVUDINE:Off label use:1.26), (ACARBOSE:Off label use:1.22), (ACETAMINOPHENDICHLORALPHENAZONEISOMETHEPTENE:Off label use:0.11), (ACETAMINOPHENTRAMADOL:Off label use:0.9), (ACLIDINIUM BROMIDE:Off label use:0.63), (AGALSIDASE BETA:Off label use:1.34), (ALBIGLUTIDE:Off label use:0.12), (ALBUTEROLIPRATROPIUM:Off label use:0.62), (ALGLUCOSIDASE ALFA:Off label use:0.53), (AMBROXOL:Off label use:0.18), (AMINOSALICYLIC ACID:Off label use:0.32), (AMLODIPINE BESYLATETELMISARTAN:Off label use:0.07), (ANTIHEMOPHILIC FACTOR, HUMAN RECOMBINANT RESIDUES 743-1636 DELETED:Off label use:0.23), (ARGININE:Off label use:0.24), (ARIPIPRAZOLE LAUROXIL:Off label use:0.59), (ASPIRINCLOPIDOGREL:Off label use:0.06), (ATAZANAVIR:Off label use:0.46), (ATENOLOLCHLORTHALIDONE:Off label use:0.04), (ATOMOXETINE:Off label use:0.01), (ATRACURIUM:Off label use:0.49), (ATRACURIUM BESYLATE:Off label use:0.39), (ATROPINE SULFATE:Off label use:0.56), (BACITRACIN:Off label use:0.58), (BECLOMETHASONEFORMOTEROL:Off label use:0.26), (BENZTROPINE:Off label use:1.05), (BERAPROST:Off label use:0.48), (BETAMETHASONE DIPROPIONATECALCIPOTRIENE:Off label use:0.01), (BETAMETHASONE DIPROPIONATECLOTRIMAZOLE:Off label use:0.29), (BETAMETHASONECALCIPOTRIENE:Off label use:0.37), (BILASTINE:Off label use:0.28), (BIPERIDEN:Off label use:0.04), (BUPRENORPHINENALOXONE:Off label use:0.29), (CABAZITAXEL:Off label use:0.2), (CALCIUM\MAGNESIUM\ZINC:Off label use:0.21), (CANAGLIFLOZINMETFORMIN HYDROCHLORIDE:Off label use:0.68), (CARBIDOPA:Off label use:0.47), (CARBIMAZOLE:Off label use:0.49), (CARBOXYMETHYLCELLULOSE SODIUM:Off label use:0.25), (CARVEDILOL PHOSPHATE:Off label use:2.06), (CEFPODOXIME PROXETIL:Off label use:0.46), (CERITINIB:Off label use:0.03), (CHLORDIAZEPOXIDE HYDROCHLORIDECLIDINIUM BROMIDE:Off label use:0.61), (CHLOROQUINECHLOROQUINE PHOSPHATE:Off label use:0.2), (CHLORPHENIRAMINEHYDROCODONE:Off label use:0.45), (CICLESONIDE:Off label use:1.21), (CIPROFIBRATE:Off label use:0.01), (CISATRACURIUM:Off label use:0.75), (CLOFARABINE:Off label use:0.71), (CODEINE PHOSPHATEGUAIFENESIN:Off label use:0.57), (COLESTIPOL:Off label use:0.04), (COLISTIMETHATE SODIUM:Off label use:1.07), (CORTISONE ACETATE:Off label use:0.13), (CROTALIDAE POLYVALENT IMMUNE FAB (OVINE):Off label use:0.48), (CYCLOPHOSPHAMIDECYTARABINEDEXAMETHASONEDOXORUBICIN METHOTREXATEVINCRISTINE:Off label use:0.03), (DABRAFENIB:Off label use:0.59), (DABRAFENIB MESYLATE:Off label use:0.02), (DACLIZUMAB:Off label use:2.2), (DAPAGLIFLOZIN PROPANEDIOL:Off label use:0.52), (DASABUVIROMBITASVIRPARITAPREVIRRITONAVIR:Off label use:0.06), (DAUNORUBICIN HYDROCHLORIDE:Off label use:0.51), (DEFERIPRONE:Off label use:0.28), (DESMOPRESSIN ACETATE:Off label use:0.6), (DEVICE:Off label use:0.57), (DEXTROAMPHETAMINE SULFATE:Off label use:0.69), (DEXTROMETHORPHAN HYDROBROMIDEQUINIDINE SULFATE:Off label use:0.13), (DIENOGEST:Off label use:0), (DIFLUNISAL:Off label use:0.05), (DIPYRONEPROMETHAZINE HYDROCHLORIDE:Off label use:0.1), (DORZOLAMIDE:Off label use:0.38), (DORZOLAMIDE HYDROCHLORIDE:Off label use:0.56), (DRONEDARONE:Off label use:0.53), (DUTASTERIDETAMSULOSIN HYDROCHLORIDE:Off label use:0.61), (ECONAZOLE:Off label use:1.53), (EDOXABAN TOSILATE MONOHYDRATE:Off label use:0.02), (EDOXABAN TOSYLATE:Off label use:2), (EFINACONAZOLE:Off label use:0.01), (ELOSULFASE ALFA:Off label use:0.1), (ELOTUZUMAB:Off label use:0.64), (ELTROMBOPAG OLAMINE:Off label use:0.44), (EMPAGLIFLOZINLINAGLIPTIN:Off label use:0.39), (EMTRICITABINETENOFOVIR:Off label use:0), (ENALAPRIL MALEATEHYDROCHLOROTHIAZIDE:Off label use:0.39), (EPINASTINE HYDROCHLORIDE:Off label use:0.82), (ERIBULIN:Off label use:0.14), (ESTRADIOLNORETHINDRONE ACETATE:Off label use:0.41), (ETHINYL ESTRADIOLGESTODENE:Off label use:0.31), (ETHINYL ESTRADIOLNORETHINDRONE:Off label use:0.65), (ETHINYL ESTRADIOLNORGESTREL:Off label use:0.43), (ETOPOSIDE PHOSPHATE:Off label use:1.37), (ETRETINATE:Off label use:0.21), (EVOLOCUMAB:Off label use:1.54), (FENOTEROL HYDROBROMIDE:Off label use:0.8), (FERROUS SULFATEGLYCINE:Off label use:0.54), (FLUOCINOLONE ACETONIDE:Off label use:0.14), (FLUOROMETHOLONE:Off label use:0.17), (FLUOROURACILLEUCOVORIN CALCIUMOXALIPLATIN:Off label use:0.18), (FORMOTEROL FUMARATE\MOMETASONE FUROATE:Off label use:0.99), (FULVESTRANT:Off label use:0.78), (FUSIDIC ACID:Off label use:1.46), (GADOBENATE DIMEGLUMINE:Off label use:0.35), (GLYCOPYRROLATEINDACATEROL MALEATE:Off label use:0.45), (HYDROCHLOROTHIAZIDEQUINAPRIL HYDROCHLORIDE:Off label use:0.57), (HYDROCODONE BITARTRATEIBUPROFEN:Off label use:0.32), (IBRITUMOMAB TIUXETAN:Off label use:1.21), (IDARUCIZUMAB:Off label use:0.66), (ILOPROST:Off label use:1.18), (IMIGLUCERASE:Off label use:0.52), (IMIPRAMINE:Off label use:0.56), (IMIQUIMOD:Off label use:0.25), (INDAPAMIDEPERINDOPRIL:Off label use:1.88), (INFLIXIMAB-DYYB:Off label use:0.18), (INTERFERON GAMMA-1B:Off label use:0.95), (IODINE:Off label use:0.42), (ISONIAZIDPYRAZINAMIDERIFAMPIN:Off label use:0.08), (LEVOMILNACIPRAN HYDROCHLORIDE:Off label use:0.18), (LEVOTHYROXINELIOTHYRONINE:Off label use:0.1), (LOMITAPIDE MESYLATE:Off label use:0.26), (MEFENAMIC ACID:Off label use:0.52), (MELPERONE:Off label use:0.04), (MEPERIDINE:Off label use:1.43), (MEPOLIZUMAB:Off label use:0.73), (METHENAMINE HIPPURATE:Off label use:1.51), (METHENOLONE ENANTHATE:Off label use:0.67), (METHYLPREDNISOLONE ACETATE:Off label use:2.63), (MIGLUSTAT:Off label use:0.44), (MILK THISTLE:Off label use:0.32), (MOMETASONE:Off label use:0.61), (MORPHINE SULFATE\NALTREXONE HYDROCHLORIDE:Off label use:0.04), (MORPHINE\MORPHINE HYDROCHLORIDE\MORPHINE SULFATE\MORPHINE TARTRATE:Off label use:0.14), (MUPIROCINMUPIROCIN CALCIUM:Off label use:1.23), (NALOXONE HYDROCHLORIDE:Off label use:0.13), (NALOXONE HYDROCHLORIDEOXYCODONE HYDROCHLORIDE:Off label use:1.82), (NALOXONE HYDROCHLORIDETILIDINE HYDROCHLORIDE:Off label use:0.67), (NARATRIPTAN HYDROCHLORIDE:Off label use:0.2), (NATEGLINIDE:Off label use:0.64), (NELARABINE:Off label use:0.09), (NINTEDANIB:Off label use:0.5), (NIRAPARIB:Off label use:0.5), (NOREPINEPHRINE BITARTRATE:Off label use:1.28), (NUSINERSEN:Off label use:0.49), (OLMESARTAN:Off label use:0.77), (OLODATEROL HYDROCHLORIDETIOTROPIUM BROMIDE MONOHYDRATE:Off label use:0.72), (ORPHENADRINE:Off label use:0.41), (OSELTAMIVIR:Off label use:1.42), (OXAPROZIN:Off label use:0.19), (PALIPERIDONE PALMITATE:Off label use:1.35), (PANITUMUMAB:Off label use:0.59), (PANTOPRAZOLE MAGNESIUM:Off label use:1.67), (PARATHYROID HORMONE:Off label use:0.33), (PEGINTERFERON BETA-1A:Off label use:1.32), (PEMETREXED:Off label use:0.88), (PHENELZINE:Off label use:0.11), (PHLOROGLUCINOL1,3,5-TRIMETHOXYBENZENE:Off label use:0.35), (PILOCARPINE:Off label use:0.56), (PILOCARPINE HYDROCHLORIDE:Off label use:0.09), (PRASUGREL HYDROCHLORIDE:Off label use:0.65), (PROCYCLIDINE:Off label use:0.08), (PROMAZINE HYDROCHLORIDE:Off label use:0.08), (PROPAFENONE HYDROCHLORIDE:Off label use:0.39), (PYRIDOXAL:Off label use:0.11), (PYRIMETHAMINE:Off label use:0), (QUINAPRILQUINAPRIL HYDROCHLORIDE:Off label use:0.11), (RADIATION THERAPY:Off label use:2), (RADIUM RA-223 DICHLORIDE:Off label use:0.4), (RALTEGRAVIR:Off label use:0.53), (RAMUCIRUMAB:Off label use:0.35), (REGORAFENIB:Off label use:0.88), (RIFABUTIN:Off label use:0.24), (RILPIVIRINE HYDROCHLORIDE:Off label use:0.21), (ROPIVACAINE HYDROCHLORIDE:Off label use:0.48), (SACUBITRILVALSARTAN:Off label use:0.63), (SAPROPTERIN DIHYDROCHLORIDE:Off label use:0.25), (SARPOGRELATE:Off label use:0.39), (SELEGILINE HYDROCHLORIDE:Off label use:0.33), (SELEXIPAG:Off label use:0.43), (SILICON DIOXIDE:Off label use:0.03), (SIPULEUCEL-T:Off label use:0.5), (SITAGLIPTIN:Off label use:1.22), (SODIUM CITRATE:Off label use:0.47), (SOFOSBUVIRVELPATASVIR:Off label use:0.3), (SOLIFENACIN:Off label use:0.11), (SUCCINYLCHOLINE:Off label use:0.12), (SUGAMMADEX:Off label use:0.31), (TEDUGLUTIDE\WATER:Off label use:0.65), (THEOPHYLLINE:Off label use:0.35), (THIOGUANINE:Off label use:0.43), (TICLOPIDINE:Off label use:0.06), (TIMOLOL MALEATE:Off label use:1.12), (TRAMETINIB:Off label use:0.42), (TRAMETINIB DIMETHYL SULFOXIDE:Off label use:0.05), (TRANDOLAPRIL:Off label use:0.58), (TRIMIPRAMINE MALEATE:Off label use:0.52), (TROSPIUM CHLORIDE:Offlabel use:1.29), (UMECLIDINIUM BROMIDE:Off label use:0.7), (VALDECOXIB:Off label use:0.02), (VARDENAFIL HYDROCHLORIDE TRIHYDRATE:Off label use:0.17), (VARICELLA-ZOSTER VIRUS STRAIN OKA/MERCK LIVE ANTIGEN:Off label use:0.23), (VECURONIUM BROMIDE:Off label use:0.85), (VEMURAFENIB:Off label use:0.38), (VISMODEGIB:Off label use:0.06), (VORTIOXETINE HYDROBROMIDE:Off label use:0.31), (ZAFIRLUKAST:Off label use:0.57), (ZILEUTON:Off label use:0.56), (ZUCLOPENTHIXOL:Off label use:0.11), (ACARBOSE:Onychomycosis:0.04), (AMIODARONE HYDROCHLORIDE:Onychomycosis:0.11), (AMITRIPTYLINE HYDROCHLORIDE:Onychomycosis:0.13), (AZITHROMYCIN DIHYDRATE:Onychomycosis:0.06), (BISACODYL OR DOCUSATE SODIUM:Onychomycosis:0.51), (BUMETANIDE:Onychomycosis:0.36), (BUPRENORPHINE HYDROCHLORIDE:Onychomycosis:0.03), (CANDESARTAN CILEXETILHYDROCHLOROTHIAZIDE:Onychomycosis:0.4), (CETIRIZINE HYDROCHLORIDEPSEUDOEPHEDRINE HYDROCHLORIDE:Onychomycosis:0.06), (CHLORTHALIDONE:Onychomycosis:0.08), (CH OLESTYRAMINE:Onychomycosis:0.83), (COLCHICINE:Onychomycosis:0.54), (DACLIZUM AB:Onychomycosis:0.11), (DALFAMPRIDINE:Onychomycosis:0.41), (DAPAGLIFLOZIN PROPANEDIOL:Onychomycosis:0.44), (DASATINIB:Onychomycosis:0.33), (DOXEPIN HYDROCHLORIDE:Onychomycosis:0.06), (DULOXETINE:Onychomycosis:0.79), (EMPAGLI FLOZINLINAGLIPTIN:Onychomycosis:0.15), (ENALAPRIL MALEATE:Onychomycosis:0.7), (ENOXAPARIN:Onychomycosis:0.18), (ENOXAPARIN SODIUM:Onychomycosis:0.16), (ESOMEPRAZOLE:Onychomycosis:0.33), (EVOLOCUMAB: Onychomycosis:1.32), (FESOTERODINE FUMARATE:Onychomycosis:0.29), (FLUOXETINEFLUOXETINE HYDROCHLORIDE:Onychomycosis:0.23), (FLUTICASONE:Onychomycosis:0.4), (GLICLAZI DE:Onychomycosis:0.13), (HYDROMORPHONE:Onychomycosis:0.07), (HYDROXYCHLORO QUINE DIPHOSPHATE:Onychomycosis:0.37), (HYDROXYZINE PAMOATE:Onychomycosis:0.66), (IBRUTINIB:Onychomycosis:0.92), (IMATINIB MESYLATE:Onychomycosis:0.09), (IMIPRAMINE HYDROCHLORIDE:Onychomycosis:0.38), (INDOMETHACIN:Onychomycosis:0.34), (INFLU ENZA VIRUS VACCINE:Onychomycosis:0.1), (INSULIN DEGLUDEC:Onychomycosis:0.06), (LEDIPASVIRSOFOSBUVIR:Onychomycosis:0.24), (LER CANIDIPINE:Onychomycosis:0.14), (LEVALBUTEROL HYDROCHLORIDE:Onychomycosis:0.27), (LINAGLIPTIN:Onychomycosis:0.58), (LITHIUM CARBONATE:Onychomycosis:0.46), (METHYLPREDNISOLONE SODIUM SUCCINATE:Onychomycosis:0.46), (METRONIDAZOLEMETRONIDAZOLE HYDROCHLORIDE:Onychomycosis:0.1), (MORPHINE:Onychomycosis:0.56), (MOXIFLOXA CIN HYDROCHLORIDE:Onychomycosis:0.07), (NABUMETONE:Onychomycosis:0.13), (NALOX ONE HYDROCHLORIDEOXYCODONE HYDROCHLORIDE:Onychomycosis:0.05), (NILOTINIB:Onychomycosis:0.21), (OXCARBAZ EPINE:Onychomycosis:0.8), (OXYBUTYNIN:Onychomycosis:0.06), (OXYBUTYNIN CHLORIDE:Onychomycosis:0.24), (PAZOPANIB HYDROCHLORIDE:Onychomycosis:0.06), (PENICILLIN:Onychomycosis:0.3), (PRAMIPEXO LE:Onychomycosis:0.41), (QUETIAPINE:Onychomycosis:0.37), (ROFLUMILAST:Onychomyc osis:0.09), (SACUBITRILVALSARTAN:Onychomycosis:0.49), (SCOPOLAMINE:Onychomyc osis:0.21), (SUMATRIPTANSUMATRIPTAN SUCCINATE:Onychomycosis:0.74), (TELMISARTAN:Onychomycosis:0.65), (TERIFLUNOMI DE:Onychomycosis:0.77), (TETRACYCLINE:Onychomycosis:0.54), (TIZANIDINE HYDROCHLORIDE:Onychomycosis:0.95), (TOCILIZUMAB:Onychomycosis:0.45), (UMECLI DINIUM BROMIDEVILANTEROL TRIFENATATE:Onychomycosis:0.4), (NAFARELIN ACETATE:Oocyte harvest:0.24), (MOXIFLOXACIN HYDROCHLORIDE:Open angle glaucoma:0.06), (POLYMYXIN B SULFATETRIMETHOPRIM SULFATE:Open angle glaucoma:0.16), (RANITIDINE HYDROCHLORIDE:Open angle glaucoma:0.04), (SPIRONOLACTONE:Open angle glaucoma:0.17), (RIFAMPIN:Ophthalmic herpes simplex:0.74), (ATAZANAVIR:Opportunistic infection prophylaxis:0.6), (ATAZANAVIRRITONAVIR:Opportunistic infection prophylaxis:0.22), (IBRITUMOMAB TIUXETAN:Opportunistic infection prophylaxis:0), (RIFAMPIN:Opportunistic infection prophylaxis:0.75), (ACENOCOUMAROL:Oral candidiasis:0.37), (ALCOHOL:Oral candidiasis:0), (ALEMTUZUMAB:Oral candidiasis:0.28), (AMIKACIN SULFATE:Oral candidiasis:0.3), (AMPHETAMINE ASPARTATEAMPHETAMINE SULFATEDEXTROAMPHETAMINE SACCHARATEDEXTROAMPHETAMINE SULFATE:Oral candidiasis:0.51), (ANASTROZOLE:Oral candidiasis:1.55), (ATAZANAVIR:Oral candidiasis:0.42), (BENAZEPRIL HYDROCHLORIDE:Oral candidiasis:0.48), (BUMETANIDE:Oral candidiasis:1.49), (BUPRENORPHINE HYDROCHLORIDE:Oral candidiasis:0.57), (BUPRENORPHINE HYDROCHLORIDENALOXONE HYDROCHLORIDE:Oral candidiasis:0.27), (CALCITONIN SALMON:Oral candidiasis:0.33), (CALCIUM ACETATE:Oral candidiasis:0.57), (CANAKINUMAB:Oral candidiasis:0.05), (CARVEDILOL PHOSPHATE:Oral candidiasis:0.25), (CETUXIMAB:Oral candidiasis:1.04), (CHLORTHALIDONE:Oral candidiasis:0.05), (CILOSTAZOL:Oral candidiasis:0.1), (DABRAFENIB:Oral candidiasis:0.29), (DALFAMPRIDINE:Oral candidiasis:0.06), (DANAZOL:Oral candidiasis:0.11), (DAPSONE:Oral candidiasis:0.43), (DARUNAVIRRITONAVIR:Oral candidiasis:0.15), (DASATINIB:Oral candidiasis:1.53), (DEXAMETHASONE ACETATE:Oral candidiasis:0.74), (DIPYRONE:Oral candidiasis:0.44), (DOCUSATE CALCIUM:Oral candidiasis:0.03), (DOFETILIDE:Oral candidiasis:0), (ECONAZOLE:Oral candidiasis:0.35), (EDOXABAN TOSYLATE:Oral candidiasis:0.47), (ELETRIPTAN HYDROBROMIDE:Oral candidiasis:0.27), (ELOTUZUMAB:Oral candidiasis:0.39), (ENALAPRIL MALEATE:Oral candidiasis:0.85), (EVOLOCUMAB:Oral candidiasis:0.13), (FESOTERODINE FUMARATE:Oral candidiasis:0.48), (FLUOCINONIDE:Oral candidiasis:0.12), (FLUOXETINEFLUOXETINE HYDROCHLORIDE:Oral candidiasis:0.65), (FORMOTEROL:Oral candidiasis:0.3), (FORMOTEROL FUMARATE:Oral candidiasis:0.64), (FORMOTEROL FUMARATE DIHYDRATE\MOMETASONE FUROATE:Oral candidiasis:0.61), (FORMOTEROL FUMARATE\MOMETASONE FUROATE:Oral candidiasis:0.15), (GEFITINIB:Oral candidiasis:0.09), (GLYBURIDE:Oral candidiasis:0.47), (HYDROCHLOROTHIAZIDETELMISARTAN:Oral candidiasis:0.21), (HYDROCHLOROTHIAZIDEVALSARTAN:Oral candidiasis:0.58), (HYDROXYUREA:Oral candidiasis:0.36), (ILOPROST:Oral candidiasis:0.28), (IMATINIB MESYLATE:Oral candidiasis:0.74), (IMIPRAMINE HYDROCHLORIDE:Oral candidiasis:0.66), (INDACATEROL:Oral candidiasis:0.01), (INDAPAMIDEPERINDOPRIL:Oral candidiasis:0.99), (INTERFERON ALFA-2A:Oral candidiasis:0.05), (IPRATROPIUM:Oral candidiasis:0.98), (IRINOTECAN HYDROCHLORIDE:Oral candidiasis:0.89), (LABETALOL HYDROCHLORIDE:Oral candidiasis:0.24), (LABETALOLLABETALOL HYDROCHLORIDE:Oral candidiasis:0.81), (LAPATINIB DITOSYLATE:Oral candidiasis:0.08), (LEDIPASVIRSOFOSBUVIR:Oral candidiasis:0.69), (LERCANIDIPINE HYDROCHLORIDE:Oral candidiasis:0.75), (LETROZOLE:Oral candidiasis:0.04), (LISDEXAMFETAMINE DIMESYLATE:Oral candidiasis:0.01), (LITHIUM CARBONATE:Oral candidiasis:0.83), (MEPOLIZUMAB:Oral candidiasis:0.19), (MESNA:Oral candidiasis:0.41), (METAMIZOLE:Oral candidiasis:0.04), (METAXALONE:Oral candidiasis:0.34), (METHENAMINE HIPPURATE:Oral candidiasis:0.05), (METHYLPREDNISOLONE ACETATE:Oral candidiasis:0.47), (MIDODRINE:Oral candidiasis:0.01), (MINOCYCLINEMINOCYCLINE HYDROCHLORIDE:Oral candidiasis:0.84), (MIRABEGRON:Oral candidiasis:0), (NEVIRAPINE:Oral candidiasis:0.19), (NILOTINIB:Oral candidiasis:2.16), (NORTRIPTYLINE:Oral candidiasis:0.17), (OMEPRAZOLE SODIUM:Oral candidiasis:0.29), (PANTOPRAZOLE MAGNESIUM:Oral candidiasis:1.15), (PEMETREXED:Oral candidiasis:0.58), (PENICILLIN G:Oral candidiasis:0.01), (PHENYTOIN:Oral candidiasis:0.77), (PHENYTOIN SODIUM:Oral candidiasis:0.11), (PREDNISOLONE SODIUM PHOSPHATE:Oral candidiasis:0.36), (PYRIDOXINE:Oral candidiasis:0.17), (RAMUCIRUMAB:Oral candidiasis:0.33), (REGORAFENIB:Oral candidiasis:0.32), (RIVASTIGMINE TARTRATE:Oral candidiasis:0.07), (RIZATRIPTAN BENZOATE:Oral candidiasis:0.33), (ROPINIROLE HYDROCHLORIDE:Oral candidiasis:0.33), (SACUBITRILVALSARTAN:Oral candidiasis:1.15), (TERBINAFINE HYDROCHLORIDE:Oral candidiasis:0.19), (TERIFLUNOMIDE:Oral candidiasis:0.22), (TETRACYCLINE:Oral candidiasis:0.4), (TIGECYCLINE:Oral candidiasis:0.29), (TOLTERODINE TARTRATE:Oral candidiasis:0.14), (TRAMETINIB:Oral candidiasis:0.36), (TRIAMTERENE:Oral candidiasis:0.2), (UMECLIDINIUM BROMIDEVILANTEROL TRIFENATATE:Oral candidiasis:0.53), (ATENOLOL:Oral contraception:0.24), (BUPRENORPHINE HYDROCHLORIDENALOXONE HYDROCHLORIDE:Oral contraception:0.12), (CHOLESTYRAMINE:Oral contraception:0.25), (CODEINE:Oral contraception:0.1), (DILTIAZEM:Oral contraception:0.23), (DIPHENHYDRAMINE:Oral contraception:0.26), (ELETRIPTAN HYDROBROMIDE:Oral contraception:0), (EVOLOCUMAB:Oral contraception:0.26), (FERROUS SULFATEFERROUS SULFATE, DRIED:Oral contraception:0.23), (HYDROXYCHLOROQUINE DIPHOSPHATE:Oral contraception:0.21), (HYDROXYZINE PAMOATE:Oral contraception:0.08), (INDOMETHACIN:Oral contraception:0.24), (ISOPROPYL ALCOHOLLEUPROLIDE ACETATENORETHINDRONE ACETATE:Oral contraception:0.68), (METHYLPREDNISOLONE ACETATE:Oral contraception:0.26), (PANTOPRAZOLE:Oral contraception:0.26), (PAROXETINEPAROXETINE HYDROCHLORIDE:Oral contraception:0.24), (PHENOBARBITAL:Oral contraception:0.19), (SIMVASTATIN:Oral contraception:0.49), (TAMSULOSIN:Oral contraception:0.11), (TIZANIDINE HYDROCHLORIDE:Oral contraception:0.2), (VALSARTAN:Oral contraception:0.14), (IBRUTINIB:Oral fungal infection:0.03), (.ALPHA.1-PROTEINASE INHIBITOR HUMAN:Oral herpes:0.13), (ACARBOSE:Oral herpes:0.17), (ACETAMINOPHENBUTALBITALCAFFEINE:Oral herpes:1.18), (ACETAMINOPHENOXYCODONEOXYCODONE HYDROCHLORIDE:Oral herpes:0.29), (ACETAZOLAMIDE:Oral herpes:0.51), (ALENDRONIC ACID:Oral herpes:0.28), (AMANTADINE:Oral herpes:0.15), (AMIODARONE:Oral herpes:0.81), (ASPARAGINASE:Oral herpes:0.56), (AZELASTINE:Oral herpes:0.01), (BENAZEPRILBENAZEPRIL HYDROCHLORIDE:Oral herpes:0.14), (BISACODYL:Oral herpes:0.38), (BRINZOLAMIDE:Oral herpes:0.09), (BUDESONIDEFORMOTEROL FUMARATE:Oral herpes:0.04), (BUPRENORPHINE HYDROCHLORIDENALOXONE HYDROCHLORIDE:Oral herpes:0.65), (CALCITONIN SALMON:Oral herpes:0.09), (CALCIUM CARBONATEVITAMIN D:Oral herpes:0.34), (CANAKINUMAB:Oral herpes:0.47), (CANDESARTAN CILEXETILHYDROCHLOROTHIAZIDE:Oral herpes:0.45), (CARVEDILOL PHOSPHATE:Oral herpes:0.37), (CETIRIZINE HYDROCHLORIDEPSEUDOEPHEDRINE HYDROCHLORIDE:Oral herpes:0.11), (CIMETIDINE:Oral herpes:0.71), (CLINDAMYCIN PHOSPHATE:Oral herpes:0.07), (CLOTRIMAZOLE:Oral herpes:0.53), (CORTICOTROPIN:Oral herpes:0.16), (DACLIZUMAB:Oral herpes:1.11), (DAPAGLIFLOZIN PROPANEDIOL:Oral herpes:0.32), (DASATINIB:Oral herpes:1.01), (DIPYRONE:Oral herpes:0.11), (DOFETILIDE:Oral herpes:0.15), (DOXAZOSIN MESYLATE:Oral herpes:0.32), (DRONABINOL:Oral herpes:0.56), (EDOXABAN TOSYLATE:Oral herpes:0.07), (EMPAGLIFLOZINLINAGLIPTIN:Oral herpes:0.19), (ERYTHROPOIETIN:Oral herpes:0.63), (ETORICOXIB:Oral herpes:0.12), (EVOLOCUMAB:Oral herpes:1.33), (FESOTERODINE FUMARATE:Oral herpes:0.44), (FLUDROCORTISONE ACETATE:Oral herpes:0.14), (FLUTICASONE FUROATESALMETEROL XINAFOATE:Oral herpes:0.23), (FORMOTEROL FUMARATE:Oral herpes:0.09), (GLYBURIDE:Oral herpes:0.26), (HERBALSTURMERIC:Oral herpes:0.43), (HYDROCHLOROTHIAZIDEVALSARTAN:Oral herpes:0.36), (HYOSCYAMINE:Oral herpes:0.33), (IBRITUMOMAB TIUXETAN:Oral herpes:0.15), (IMATINIB MESYLATE:Oral herpes:0.93), (IMIPRAMINE HYDROCHLORIDE:Oral herpes:0.51), (INSULIN NOS:Oral herpes:1.05), (IPRATROPIUM:Oral herpes:0.65), (IRINOTECAN:Oral herpes:0.03), (LEDIPASVIRSOFOSBUVIR:Oral herpes:0.57), (LERCANIDIPINE:Oral herpes:0.3), (LERCANIDIPINE HYDROCHLORIDE:Oral herpes:0.08), (LINAGLIPTIN:Oral herpes:0.59), (LITHIUM CARBONATE:Oral herpes:0.94), (MECLIZINE HYDROCHLORIDE:Oral herpes:0.21), (MEGESTROL ACETATE:Oral herpes:0.15), (METHENAMINE HIPPURATE:Oral herpes:0.45), (METHYLPREDNISOLONE ACETATE:Oral herpes:1.08), (MOXIFLOXACIN:Oral herpes:0.32), (NADOLOL:Oral herpes:0.24), (OMEGA-3-ACID ETHYL ESTERS:Oral herpes:0.69), (PANTOPRAZOLE MAGNESIUM:Oral herpes:0.93), (PAZOPANIB HYDROCHLORIDE:Oral herpes:0.46), (PERINDOPRIL:Oral herpes:0.31), (POSACONAZOLE:Oral herpes:0.61), (PRAMIPEXOLE DIHYDROCHLORIDE:Oral herpes:0.69), (QUETIAPINE:Oral herpes:0.85), (RABEPRAZOLE:Oral herpes:0.5), (RIZATRIPTAN:Oral herpes:0.08), (ROFLUMILAST:Oral herpes:0.48), (TERBINAFINE HYDROCHLORIDE:Oral herpes:0.22), (TERBUTALINE SULFATE:Oral herpes:0.33), (TETRACYCLINE:Oral herpes:0.35), (TIOTROPIUM:Oral herpes:0.11), (TRIAMTERENE:Oral herpes:0.58), (UMECLIDINIUM BROMIDEVILANTEROL TRIFENATATE:Oral herpes:0.61), (VANCOMYCIN HYDROCHLORIDE:Oral herpes:0.49), (VINCRISTINE SULFATE:Oral herpes:0.67), (ZONISAMIDE:Oral herpes:0.05), (ACETAMINOPHENBUTALBITALCAFFEINE:Oral pain:0.21), (AMIODARONE HYDROCHLORIDE:Oral pain:0.21), (AMPHETAMINE ASPARTATEAMPHETAMINE SULFATEDEXTROAMPHETAMINE SACCHARATEDEXTROAMPHETAMINE SULFATE:Oral pain:0.51), (CALCITRIOL:Oral pain:0.28), (CALCIUM ACETATE:Oral pain:0.03), (CALCIUMVITAMIN D:Oral pain:0.58), (CANDESARTAN CILEXETILHYDROCHLOROTHIAZIDE:Oral pain:0.12), (CILASTATIN SODIUMIMIPENEM:Oral pain:0.13), (CIMETIDINE:Oral pain:0.17), (CLOZAPINE:Oral pain:0.43), (DILTIAZEM:Oral pain:0.62), (DILTIAZEM HYDROCHLORIDE:Oral pain:1.3), (ELETRIPTAN HYDROBROMIDE:Oral pain:0.09), (EPIRUBICIN:Oral pain:0.06), (EVOLOCUMAB:Oral pain:0.31), (EZETIMIBE:Oral pain:0.18), (FERROUS SULFATEFERROUS SULFATE, DRIED:Oral pain:1.12), (FULVESTRANT:Oral pain:0.03), (IMATINIB MESYLATE:Oral pain:0.44), (INDOMETHACIN:Oral pain:0.36), (IRBESARTAN:Oral pain:0.02), (IRINOTECAN:Oral pain:0.15), (LEDIPASVIRSOFOSBUVIR:Oral pain:0.2), (LEFLUNOMIDE:Oral pain:0.02), (LEVALBUTEROL HYDROCHLORIDE:Oral pain:0.02), (LISDEXAMFETAMINE DIMESYLATE:Oral pain:0.47), (METAXALONE:Oral pain:0.26), (MOXIFLOXACIN HYDROCHLORIDE:Oral pain:0.39), (NILOTINIB:Oral pain:0.72), (OXCARBAZEPINE:Oral pain:0.9), (OXYGEN:Oral pain:0.32), (PANTOPRAZOLE MAGNESIUM:Oral pain:0.12), (PAROXETINEPAROXETINE HYDROCHLORIDE:Oral pain:0.17), (PHENOBARBITAL:Oral pain:0.15), (PRAVASTATIN SODIUM:Oral pain:0.41), (PRIMIDONE:Oral pain:0.23), (QUETIAPINE:Oral pain:0.16), (RABEPRAZOLE:Oral pain:0.02), (RABEPRAZOLE SODIUM:Oral pain:0.05), (RANOLAZINE:Oral pain:0.08), (RIZATRIPTAN BENZOATE:Oral pain:0.05), (SOTALOL:Oral pain:0.19), (SULFAMETHOXAZOLE:Oral pain:0.21), (TOCILIZUMAB:Oral pain:0.33), (UMECLIDINIUM BROMIDEVILANTEROL TRIFENATATE:Oral pain:0.04), (VERAPAMIL HYDROCHLORIDE:Oral pain:0.8), (VITAMIN B:Oral pain:0.17), (PYRAZINAMIDE:Orchitis:0.57), (PYRAZINAMIDE:Organising pneumonia:0.6), (ACETAMINOPHENOXYCODONEOXYCODONE HYDROCHLORIDE:Oropharyngeal pain:0.17), (ALEMTUZUMAB:Oropharyngeal pain:0.12), (AMISULPRIDE:Oropharyngeal pain:0.03), (AMPICILLIN SODIUMSULBACTAM SODIUM:Oropharyngeal pain:0.26), (ARIPIPRAZOLE:Oropharyngeal pain:0.45), (BENAZEPRILBENAZEPRIL HYDROCHLORIDE:Oropharyngeal pain:0.2), (BRINZOLAMIDE:Oropharyngeal pain:0.44), (BUDESONIDEFORMOTEROL FUMARATE:Oropharyngeal pain:0.23), (CALCIUM ACETATE:Oropharyngeal pain:0.33), (CALCIUM CARBONATEVITAMIN D:Oropharyngeal pain:0.34), (CANAKINUMAB:Oropharyngeal pain:0.5), (CANDESARTAN CILEXETIL:Oropharyngeal pain:1.09), (CANDESARTAN CILEXETILHYDROCHLOROTHIAZIDE:Oropharyngeal pain:0.66), (CARVEDILOL PHOSPHATE:Oropharyngeal pain:0.52), (CEFOTAXIME SODIUM:Oropharyngeal pain:0.24), (CHLORAMBUCIL:Oropharyngeal pain:0.04), (CHOLESTYRAMINE:Oropharyngeal pain:1.1), (CICLESONIDE:Oropharyngeal pain:0.13), (CILASTATIN SODIUMIMIPENEM:Oropharyngeal pain:0.67), (CORTICOTROPIN:Oropharyngeal pain:0.01), (DACLIZUMAB:Oropharyngeal pain:0.4), (DALFAMPRIDINE:Oropharyngeal pain:0.81), (DALTEPARIN SODIUM:Oropharyngeal pain:0.18), (DESLORATADINE:Oropharyngeal pain:0.66), (DEXAMETHASONE ACETATE:Oropharyngeal pain:0.17), (DIVALPROEX SODIUM:Oropharyngeal pain:0.99), (DOCUSATE CALCIUM:Oropharyngeal pain:0.31), (DOFETILIDE:Oropharyngeal pain:0.01), (DOXAZOSINDOXAZOSIN MESYLATE:Oropharyngeal pain:0.39), (DOXEPIN:Oropharyngeal pain:0.18), (ELETRIPTAN HYDROBROMIDE:Oropharyngeal pain:0.8), (ENALAPRIL MALEATE:Oropharyngeal pain:0.83), (ERYTHROPOIETIN:Oropharyngeal pain:0.85), (ESOMEPRAZOLE MAGNESIUMNAPROXEN:Oropharyngeal pain:0.05), (ESZOPICLONE:Oropharyngeal pain:1.22), (EVOLOCUMAB:Oropharyngeal pain:0.62), (FESOTERODINE FUMARATE:Oropharyngeal pain:0.35), (FLUDROCORTISONE ACETATE:Oropharyngeal pain:0.01), (FLUOXETINEFLUOXETINE HYDROCHLORIDE:Oropharyngeal pain:0.71), (FLUTICASONE FUROATESALMETEROL XINAFOATE:Oropharyngeal pain:0.55), (FORMOTEROL FUMARATE:Oropharyngeal pain:0.34), (FORMOTEROL FUMARATE DIHYDRATE\MOMETASONE FUROATE:Oropharyngeal pain:0.7), (FUSIDIC ACID:Oropharyngeal pain:0.05), (GIMERACILOTERACILTEGAFUR:Oropharyngeal pain:0.02), (GLYCOPYRRONIUMINDACATEROL:Oropharyngeal pain:0.27), (HYDROCHLOROTHIAZIDEIRBESARTAN:Oropharyngeal pain:0.5), (HYDROXYCHLOROQUINE DIPHOSPHATE:Oropharyngeal pain:0.27), (HYOSCYAMINE:Oropharyngeal pain:0.03), (IMATINIB MESYLATE:Oropharyngeal pain:1), (IMIPRAMINE HYDROCHLORIDE:Oropharyngeal pain:0.85), (INDAPAMIDE:Oropharyngeal pain:0.3), (INDAPAMIDEPERINDOPRIL:Oropharyngeal pain:0.25), (INFLIXIMAB-DYYB:Oropharyngeal pain:0.07), (IRINOTECAN HYDROCHLORIDE:Oropharyngeal pain:0.81), (ISOSORBIDE:Oropharyngeal pain:0.45), (KETOROLACKETOROLAC TROMETHAMINE:Oropharyngeal pain:0.05), (LAPATINIB DITOSYLATE:Oropharyngeal pain:0.07), (LERCANIDIPINE:Oropharyngeal pain:0.56), (LERCANIDIPINE HYDROCHLORIDE:Oropharyngeal pain:0.23), (LETROZOLE:Oropharyngeal pain:0.53), (LEVALBUTEROL:Oropharyngeal pain:0), (LEVOLEUCOVORIN CALCIUM:Oropharyngeal pain:0.35), (MESNA:Oropharyngeal pain:0.23), (METAXALONE:Oropharyngeal pain:0.49), (METHENAMINE HIPPURATE:Oropharyngeal pain:0.37), (METHYLPREDNISOLONE ACETATE:Oropharyngeal pain:0.61), (NADOLOL:Oropharyngeal pain:0.12), (NALOXONE HYDROCHLORIDEOXYCODONE HYDROCHLORIDE:Oropharyngeal pain:0.48), (NINTEDANIB:Oropharyngeal pain:0.22), (NORTRIPTYLINE:Oropharyngeal pain:0.6), (OLAPARIB:Oropharyngeal pain:0.01), (OLODATEROL HYDROCHLORIDETIOTROPIUM BROMIDE MONOHYDRATE:Oropharyngeal pain:0.45), (OMEGA-3-ACID ETHYL ESTERS:Oropharyngeal pain:0.3), (OXYBUTYNIN CHLORIDE:Oropharyngeal pain:0.38), (PANTOPRAZOLE MAGNESIUM:Oropharyngeal pain:0.96), (PAROXETINE:Oropharyngeal pain:0.82), (PAROXETINEPAROXETINE HYDROCHLORIDE:Oropharyngeal pain:0.75), (PAZOPANIB HYDROCHLORIDE:Oropharyngeal pain:0.39), (PEMETREXED DISODIUM:Oropharyngeal pain:0.47), (PHENOBARBITAL:Oropharyngeal pain:0.02), (PHENYTOIN SODIUM:Oropharyngeal pain:0.13), (POSACONAZOLE:Oropharyngeal pain:0.36), (PRIMIDONE:Oropharyngeal pain:0.87), (RAMUCIRUMAB:Oropharyngeal pain:0.02), (RANOLAZINE:Oropharyngeal pain:0.53), (RIZATRIPTAN BENZOATE:Oropharyngeal pain:0.7), (ROSUVASTATIN:Oropharyngeal pain:0.05), (SODIUM OXYBATE:Oropharyngeal pain:0.81), (SUMATRIPTANSUMATRIPTAN SUCCINATE:Oropharyngeal pain:0.78), (SUNITINIB MALATE:Oropharyngeal pain:0.09), (TERBUTALINE SULFATE:Oropharyngeal pain:0.34), (TERIFLUNOMIDE:Oropharyngeal pain:0.36), (THALIDOMIDE:Oropharyngeal pain:1.17), (TOLTERODINE TARTRATE:Oropharyngeal pain:0.44), (UMECLIDINIUM BROMIDEVILANTEROL TRIFENATATE:Oropharyngeal pain:0.27), (VILAZODONE HYDROCHLORIDE:Oropharyngeal pain:0.06), (VINCRISTINE SULFATE:Oropharyngeal pain:0.27), (VINORELBINE TARTRATE:Oropharyngeal pain:0.09), (VITAMIN A:Oropharyngeal pain:0.07), (ZINC:Oropharyngeal pain:0.11), (.ALPHA.1-PROTEINASE INHIBITOR HUMAN:Orthostatic hypotension:0.01), (ACETAMINOPHENOXYCODONE HYDROCHLORIDEOXYCODONE TEREPHTHALATE:Orthostatic hypotension:0.15), (ACETAMINOPHENOXYCODONEOXYCODONE HYDROCHLORIDE:Orthostatic hypotension:0.43), (ACETAMINOPHENTRAMADOL:Orthostatic hypotension:0.07), (ALCOHOL:Orthostatic hypotension:0.14), (AMISULPRIDE:Orthostatic hypotension:0.26), (BENAZEPRIL HYDROCHLORIDE:Orthostatic hypotension:1.06), (BENAZEPRILBENAZEPRIL HYDROCHLORIDE:Orthostatic hypotension:0.28), (BRINZOLAMIDE:Orthostatic hypotension:0.7), (BUDESONIDEFORMOTEROL FUMARATE:Orthostatic hypotension:0.1), (BUPRENORPHINE HYDROCHLORIDENALOXONE HYDROCHLORIDE:Orthostatic hypotension:0.15), (CALCIUM CARBONATEVITAMIN D:Orthostatic hypotension:0.13), (CALCIUM\MAGNESIUM:Orthostatic hypotension:0.02), (CALCIUM\MAGNESIUM\ZINC:Orthostatic hypotension:0), (CANDESARTAN CILEXETILHYDROCHLOROTHIAZIDE:Orthostatic hypotension:0.58), (CARVEDILOL PHOSPHATE:Orthostatic hypotension:1.05), (CERTOLIZUMAB PEGOL:Orthostatic hypotension:0.55), (CETIRIZINE HYDROCHLORIDEPSEUDOEPHEDRINE HYDROCHLORIDE:Orthostatic hypotension:0.04), (CHLORPROMAZINE:Orthostatic hypotension:0), (CHONDROITIN SULFATE (BOVINE)GLUCOSAMINE HYDROCHLORIDE:Orthostatic hypotension:0.16), (CIMETIDINE:Orthostatic hypotension:0.29), (CIPROFLOXACIN HYDROCHLORIDE:Orthostatic hypotension:0.77), (CLINDAMYCINCLINDAMYCIN PHOSPHATE:Orthostatic hypotension:0.71), (CLOBAZAM:Orthostatic hypotension:0.11), (COD LIVER OIL:Orthostatic hypotension:0.06), (CORTICOTROPIN:Orthostatic hypotension:0.24), (CORTISONEHYDROCORTISONE:Orthostatic hypotension:0.47), (DACLIZUMAB:Orthostatic hypotension:0.93), (DANAZOL:Orthostatic hypotension:0.08), (DAPAGLIFLOZIN PROPANEDIOL:Orthostatic hypotension:0.32), (DASATINIB:Orthostatic hypotension:0.79), (DEFLAZACORT:Orthostatic hypotension:0.24), (DESLORATADINE:Orthostatic hypotension:0.46), (DEVICE:Orthostatic hypotension:0.36), (DIMENHYDRINATE:Orthostatic hypotension:0.3), (DOXEPIN HYDROCHLORIDE:Orthostatic hypotension:0.17), (EDOXABAN TOSYLATE:Orthostatic hypotension:0.17), (EMPAGLIFLOZINLINAGLIPTIN:Orthostatic hypotension:0.19), (ENALAPRIL:Orthostatic hypotension:1.86), (ERYTHROMYCIN:Orthostatic hypotension:0.07), (EVEROLIMUS:Orthostatic hypotension:0.57), (FLUVOXAMINE MALEATE:Orthostatic hypotension:0.19), (GLYCOPYRRONIUMINDACATEROL:Orthostatic hypotension:0.05), (GUANFACINE:Orthostatic hypotension:0.21), (HYALURONATE SODIUM:Orthostatic hypotension:0.18), (HYDROCHLOROTHIAZIDEIRBESARTAN:Orthostatic hypotension:0.32), (HYDROCHLOROTHIAZIDELISINOPRIL:Orthostatic hypotension:1.22), (HYDROCHLOROTHIAZIDELOSARTAN POTASSIUM:Orthostatic hypotension:0.53), (HYDROCHLOROTHIAZIDETELMISARTAN:Orthostatic hypotension:0.24), (HYDROCHLOROTHIAZIDEVALSARTAN:Orthostatic hypotension:0.81), (HYDROMORPHONE:Orthostatic hypotension:0.47), (ILOPROST:Orthostatic hypotension:0.14), (IMIPRAMINE HYDROCHLORIDE:Orthostatic hypotension:0.61), (INDAPAMIDEPERINDOPRIL:Orthostatic hypotension:0.03), (INDOMETHACIN:Orthostatic hypotension:0.24), (INSULIN GLULISINE:Orthostatic hypotension:0.13), (LERCANIDIPINE:Orthostatic hypotension:0.63), (LERCANIDIPINE HYDROCHLORIDE:Orthostatic hypotension:0.07), (LEVOMEFOLATE CALCIUM:Orthostatic hypotension:0.05), (LIDOCAINE HYDROCHLORIDE:Orthostatic hypotension:0.43), (LISDEXAMFETAMINE DIMESYLATE:Orthostatic hypotension:1.04), (LOMITAPIDE MESYLATE:Orthostatic hypotension:0.32), (METHIMAZOLE:Orthostatic hypotension:0.06), (METHYLPREDNISOLONE SODIUM SUCCINATE:Orthostatic hypotension:1.37), (MOXIFLOXACIN HYDROCHLORIDE:Orthostatic hypotension:1.23), (NABUMETONE:Orthostatic hypotension:0.26), (NALTREXONE:Orthostatic hypotension:0.08), (NATEGLINIDE:Orthostatic hypotension:0.11), (NEBIVOLOL:Orthostatic hypotension:0.5), (NILOTINIB:Orthostatic hypotension:0.58), (PANTOPRAZOLE MAGNESIUM:Orthostatic hypotension:0.64), (PATIROMER:Orthostatic hypotension:0.3), (PEGINTERFERON BETA-1A:Orthostatic hypotension:0.47), (PHENYTOIN SODIUM:Orthostatic hypotension:0.03), (PONATINIB:Orthostatic hypotension:0.16), (PRAZOSIN:Orthostatic hypotension:0.4), (ROFLUMILAST:Orthostatic hypotension:0.34), (SACUBITRILVALSARTAN:Orthostatic hypotension:1.09), (SECUKINUMAB:Orthostatic hypotension:0.19), (SODIUM PHOSPHATE, DIBASIC\SODIUM PHOSPHATE, MONOBASIC:Orthostatic hypotension:0), (SULFAMETHOXAZOLE:Orthostatic hypotension:0.14), (TELMISARTAN:Orthostatic hypotension:1.11), (TERBUTALINE SULFATE:Orthostatic hypotension:0.01), (TIOTROPIUM:Orthostatic hypotension:0.19), (TRIAMTERENE:Orthostatic hypotension:0.66), (VARENICLINE TARTRATE:Orthostatic hypotension:1.75), (ACETAMINOPHENDICHLORALPHENAZONEISOMETHEPTENE:Ost eoarthritis:0.14), (AGALSIDASE BETA:Osteoarthritis:1.98), (AJMALINE:Osteoarthritis:0.01), (ALISKIREN HEMIFUMARATEHYDROCHLOROTHIAZIDE:Osteoarthritis:0.13), (AMBROXOL:Osteoart hritis:0.39), (AMINO ACIDSDEXTROSEELECTROLYTES NOS:Osteoarthritis:0.46), (AMISULPRIDE:Osteoarthritis:1.43), (AMMONIUM CHLORIDEDEXTROMETHORPHAN\SODIUM CITRATE:Osteoarthritis:0.32), (AMOXAPINE:Osteoarthritis:0.12), (AMPICILLIN SODIUMSULBACTAM SODIUM:Osteoarthritis:0.92), (ANAGRELIDE HYDROCHLORIDE:Osteoarthritis:0.39), (ANTIHISTAMINES NOS:Osteoarthritis:0.76), (ARIPIPRAZOLE LAUROXIL:Osteoarthritis:0.26), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Osteoarthritis:0.32), (ASCORBIC ACIDBIOTINCYANOCOBALAMINFOLIC ACIDNIACINPANTOTHENIC ACIDPYRIDOXINE\RIBOFLAVINTHIAMINE:Osteoarthritis:0.17), (ASPIRINCLOPIDOGR EL:Osteoarthritis:0.43), (ASUNAPREVIR:Osteoarthritis:0.94), (ATOVAQUONE:Osteoarthritis: 0.47), (ATROPINE SULFATE:Osteoarthritis:0.38), (AZITHROMYCIN OR METHOTREXATE SODIUM:Osteoarthritis:0.34), (BAMIFYLLINE HYDROCHLORIDE:Osteoarthritis:0.55), (BECLOMETHASONE DIPROPIONATEFORMOTEROL:Osteoarthritis:0.4), (BENSERAZIDELEVODOPA:Osteoart hritis:0.51), (BENZOCAINE:Osteoarthritis:0.17), (BEPRIDIL:Osteoarthritis:0.74), (BETAHISTI NE MESILATE:Osteoarthritis:0.19), (BETAMETHASONE DIPROPIONATECALCIPOTRIENE:Osteoarthritis:0.64), (BETHANECHOL CHLORIDE:Osteoarthritis:0.77), (BICARBONATE ION:Osteoarthritis:0.21), (BLINATUMOMAB:Osteoarthritis:0.15), (BOTULINUM TOXIN TYPE A:Osteoarthritis:0.43), (BUCILLAMINE:Osteoarthritis:0.01), (CABAZITAXEL:Osteoarthritis:0.51), (CALCIUM CARBONATECHOLECALCIFEROLMAGNESIUM CARBONATE:Osteoarthritis:0.35), (CARTEOLOL:Osteoarthritis:0.28), (CARTEOLOL HYDROCHLORIDE:Osteoarthritis:0.69), (CEFPODOXIME PROXETIL:Osteoarthritis:0.6), (CEFPROZIL:Osteoarthritis:0.06), (CELIPROLOL:Osteoarthritis: 0.26), (CERIVASTATIN SODIUM:Osteoarthritis:0.34), (CERTOPARIN SODIUM:Osteoarthritis:0.6), (CHLORDIAZEPOXIDE:Osteoarthritis:0.13), (CLEMASTINE FUMARATE:Osteoarthritis:0.07), (CRANBERRY JUICE:Osteoarthritis:0.29), (CYANOCOBALAMINFOLIC ACIDPYRIDOXINE:Osteoarthritis:0.26), (CYPROTERONE ACETATE:Osteoarthritis:0.05), (DABRAFENIB:Osteoarthritis:0.42), (DABRAFENIB MESYLATE:Osteoarthritis:0.13), (DACLIZUMAB:Osteoarthritis:1.79), (DANAZOL:Osteoarthri tis:1.16), (DANTROLENE SODIUM:Osteoarthritis:0.51), (DARUNAVIR ETHANOLATE:Osteoarthritis:0.08), (DEGARELIX ACETATE:Osteoarthritis:0.21), (DESOXIMETASONE:Osteoarthritis:0.69), (DESVENLAFAXI NE:Osteoarthritis:0.08), (DEXAMETHASONE ACETATE:Osteoarthritis:1.43), (DISOPYRAMIDE:Osteoarthritis:0.13), (DOLASETRON MESYLATE:Osteoarthritis:0.67), (DROXIDOPA:Osteoarthritis:0.4), (ECONAZOLE:Osteoarthri tis:1.97), (ELBASVIRGRAZOPREVIR:Osteoarthritis:0.28), (ELOTUZUMAB:Osteoarthritis:0.86), (EMPAGLIFLOZINLINAGLIPTIN:Osteoarthritis:1.65), (ERIBULIN:Osteoarthritis:0.07), (E THACRYNIC ACID:Osteoarthritis:0.47), (ETHINYL ESTRADIOLNORGESTREL:Osteoarthritis:0.63), (ETRETINATE:Osteoarthritis:0.97), (FENO TEROL HYDROBROMIDE:Osteoarthritis:1.31), (FERROUS BISGLYCINATE:Osteoarthritis:0.68), (FERROUS CITRATE:Osteoarthritis:0.79), (FERROUS GLYCINE SULFATE:Osteoarthritis:0.21), (FERROUS SULFATEGLYCINE:Osteoarthritis:0.79), (FLUNARIZINE HYDROCHLORIDE:Osteoarthritis:0.28), (FLUTICASONE PROPIONATEFORMOTEROL FUMARATE:Osteoarthritis:0.25), (FLUVOXAMINE:Osteoarthritis:0.12), (FONDAPARINUX: Osteoarthritis:0.75), (FOSFOMYCIN:Osteoarthritis:0.19), (GADOLINIUM:Osteoarthritis:0.72), (GEMCITABINE HYDROCHLORIDE:Osteoarthritis:0.38), (GIMERACILOTERACILTEGAFUR:Osteoarthritis: 0.23), (GLIMEPIRIDEROSIGLITAZONE MALEATE:Osteoarthritis:0.33), (GLUTAMINE:Osteoarthritis:0.25), (GLYCOPYRROLATE:Os teoarthritis:1.26), (GLYCOPYRROLATEINDACATEROL MALEATE:Osteoarthritis:0.8), (GLYCOPYRRONIUM:Osteoarthritis:0.79), (GOLD:Osteoarthrit is:0.08), (GUANFACINE:Osteoarthritis:0.69), (HYDROCHLOROTHIAZIDEMETOPROLOL SUCCINATE:Osteoarthritis:0.49), (HYDROCODONE BITARTRATEIBUPROFEN:Osteoarthritis:1.06), (HYDROCORTISONE SODIUM SUCCINATE:Osteoarthritis:0.78), (HYOSCYAMINE SULFATEMETHENAMINEMETHYLENE BLUEPHENYL SALICYLATE\SODIUM PHOSPHATE, MONOBASIC, MONOHYDRATE:Osteoarthritis:0.23), (IDARUCIZUMAB:Osteoarthritis:0.15), (ILOPROST:0 steoarthritis:1.61), (IMIGLUCERASE:Osteoarthritis:0.53), (INTERFERON:Osteoarthritis:1.09), (INTERFERON ALFA-2A:Osteoarthritis:1.42), (INTERFERON ALFA-2B:Osteoarthritis:1.04), (INTERFERON GAMMA-1B:Osteoarthritis:0.43), (IRON SUCROSE:Osteoarthritis:1.16), (IRONPOLYSACCHARIDES:Osteoarthritis:0.47), (LACTOBA CILLUS RHAMNOSUS:Osteoarthritis:1), (LANREOTIDE:Osteoarthritis:0.31), (LANREOTIDE ACETATE:Osteoarthritis:0.91), (LAPATINIB DITOSYLATE:Osteoarthritis:0.55), (LEVALBUTEROL TARTRATE:Osteoarthritis:0.15), (LEVOTHYROXINE SODIUMPOTASSIUM IODIDE:Osteoarthritis:0.71), (LINSIDOMINE:Osteoarthritis:0.25), (LUTETIUM LU-177:Osteoarthritis:0.34), (MAGNESIUM ASPARTATE HYDROCHLORIDE:Osteoarthritis:0.72), (MAGNESIUM ASPARTATE\POTASSIUM ASPARTATE:Osteoarthritis:0.2), (MANIDIPINE HYDROCHLORIDE:Osteoarthritis:0.04), (MELPERONE:Osteoarthritis:0.37), (MEPERIDINE: Osteoarthritis:0.95), (METFORMIN HYDROCHLORIDESAXAGLIPTIN:Osteoarthritis:0.35), (METHENAMINE MANDELATE:Osteoarthritis:0.24), (METHENOLONE ENANTHATE:Osteoarthritis:0.2), (METHOXY POLYETHYLENE GLYCOL-EPOETIN BETA:Osteoarthritis:0.08), (METRELEPTIN:Osteoarthritis:0.72), (MEXILETINE:Osteoarthritis: 0.16), (MICAFUNGIN:Osteoarthritis:0.01), (MIGLUSTAT:Osteoarthritis:0.17), (MINOCYCLIN E HYDROCHLORIDE:Osteoarthritis:1.52), (MOXONIDINE:Osteoarthritis:1.77), (NAPROXEN SODIUMSUMATRIPTAN SUCCINATE:Osteoarthritis:0), (NATEGLINIDE:Osteoarthritis:1.6), (NISOLDIPINE:Osteoarthri tis:0.32), (OCTREOTIDE:Osteoarthritis:1.04), (ORPHENADRINE:Osteoarthritis:1.13), (OSELT AMIVIR:Osteoarthritis:0.85), (OXYMORPHONE:Osteoarthritis:0.24), (PARICALCITOL:Osteo arthritis:0.61), (PATIROMER:Osteoarthritis:0.8), (PENICILLAMINE:Osteoarthritis:0.04), (PENI CILLIN G:Osteoarthritis:0.69), (PIMOBENDAN:Osteoarthritis:0.29), (PINDOLOL:Osteoarthritis:0), (PIP ERACILLIN:Osteoarthritis:0.07), (PIZOTYLINE:Osteoarthritis:0.03), (POLYETHYLENE GLYCOL 3350POTASSIUM CHLORIDE\SODIUM BICARBONATE\SODIUM CHLORIDE\SODIUM SULFATE ANHYDROUS:Osteoarthritis:0.75), (PONATINIB:Osteoarthritis:1.96), (POVIDONE:Osteoarthri tis:0.38), (PREDNISOLONE SODIUM SUCCINATE:Osteoarthritis:0.14), (PROTRIPTYLINE HYDROCHLORIDE:Osteoarthritis:0.82), (PRUCALOPRIDE SUCCINATE:Osteoarthritis:0.28), (QUINACRINE:Osteoarthritis:0.85), (RADIATION THERAPY:Osteoarthritis:1.16), (RAMUCIRUMAB:Osteoarthritis:0.1), (RILPIVIRINE HYDROCHLORIDE:Osteoarthritis:0.41), (RIZATRIPTAN:Osteoarthritis:1), (SCOPOLAMINE: Osteoarthritis:1.88), (SELEXIPAG:Osteoarthritis:0.51), (SEVELAMER HYDROCHLORIDE:Osteoarthritis:0.93), (SIBUTRAMINE HYDROCHLORIDE:Osteoarthritis:0.4), (SUFENTANIL CITRATE:Osteoarthritis:0.07), (TAFAMIDIS MEGLUMINE:Osteoarthritis:0.23), (TAMOXIFEN CITRATE:Osteoarthritis:0.51), (TAZOBACTAM:Osteoarthritis:0.43), (TEDUGLUTIDEWATE R:Osteoarthritis:0.24), (TELOTRISTAT ETHYL:Osteoarthritis:0.2), (TERBUTALINE:Osteoarthritis:0.94), (TESTOSTERONE UNDECANOATE:Osteoarthritis:1.64), (THIOCTIC ACID:Osteoarthritis:0.75), (TIBOLONE:Osteoarthritis:0.41), (TILIDINE:Osteoarthritis:0.29), (TI MOLOLTRAVOPROST:Osteoarthritis:0.6), (TOFACITINIB:Osteoarthritis:0.25), (TRAMETIN 1B:Osteoarthritis:0.27), (TRANDOLAPRIL:Osteoarthritis:1.16), (TREPROSTINIL DIOLAMINE:Osteoarthritis:0.05), (TRIMIPRAMINE MALEATE:Osteoarthritis:1.33), (TRIPTORELIN PAMOATE:Osteoarthritis:0.77), (TROSPIUM:Osteoarthritis:0.04), (UBIQUINOL:Osteoarthritis: 0.09), (VALGANCICLOVIR HYDROCHLORIDE:Osteoarthritis:1.66), (VINCRISTINE SULFATE:Osteoarthritis:1), (WARFARIN POTASSIUM:Osteoarthritis:0.64), (YEAST:Osteoarthritis:0.81), (ZILEUTON:Osteoarthritis:0.66), (ALBUTEROL SULFATEIPRATROPIUM BROMIDE:Osteomyelitis:0.35), (ALCOHOL:Osteomyelitis:0.16), (ARMODAFINIL:Osteomyeli tis:0.17), (BENAZEPRIL HYDROCHLORIDE:Osteomyelitis:0.15), (CALCIUMVITAMIN D:Osteomyelitis:0.25), (CANDESARTAN CILEXETILHYDROCHLOROTHIAZIDE:Osteomyelitis:0.52), (CARVEDILOL PHOSPHATE:Osteomyelitis:0.1), (CASPOFUNGIN:Osteomyelitis:0.63), (CERTOLIZUMAB PEGOL:Osteomyelitis:0.78), (CLOTRIMAZOLE:Osteomyelitis:0.37), (CORTISONEHYDROC ORTISONE:Osteomyelitis:0.1), (DAPAGLIFLOZIN PROPANEDIOL:Osteomyelitis:0.17), (DASATINIB:Osteomyelitis:0.34), (DEVICE:Osteomyelit is:0.3), (DEXLANSOPRAZOLE:Osteomyelitis:1.13), (DIVALPROEX SODIUM:Osteomyelitis:0.65), (ENALAPRIL MALEATE:Osteomyelitis:0.33), (EVOLOCUMAB:Osteomyelitis:0.71), (FILGRASTIM:Osteom yelitis:0.15), (FINGOLIMOD HYDROCHLORIDE:Osteomyelitis:0.14), (FLUTICASONE:Osteomyelitis:0.15), (HYDROCHL OROTHIAZIDEVALSARTAN:Osteomyelitis:0.16), (HYDROXYCHLOROQUINE DIPHOSPHATE:Osteomyelitis:0.09), (ILOPROST:Osteomyelitis:0.14), (INDAPAMIDEPERIN DOPRIL:Osteomyelitis:0.34), (IPRATROPIUM:Osteomyelitis:0.33), (LERCANIDIPINE:Osteom yelitis:0.64), (LERCANIDIPINE HYDROCHLORIDE:Osteomyelitis:0.09), (LITHIUM CARBONATE:Osteomyelitis:0.5), (METAXALONE:Osteomyelitis:0.21), (METHIMAZOLE:Os teomyelitis:0.12), (METHYLPREDNISOLONE SODIUM SUCCINATE:Osteomyelitis:1.3), (NALOXONE HYDROCHLORIDEOXYCODONE HYDROCHLORIDE:Osteomyelitis:0.23), (NILOTINIB:Osteomyelitis:0.53), (OMEGA-3-ACID ETHYL ESTERS:Osteomyelitis:0.02), (OXCARBAZEPINE:Osteomyelitis:0.97), (POSACONAZOLE:Os teomyelitis:0.64), (PRAMIPEXOLE DIHYDROCHLORIDE:Osteomyelitis:0.17), (PRIMIDONE:Osteomyelitis:0.39), (PYRIDOXINE: Osteomyelitis:0.51), (RANITIDINE HYDROCHLORIDE:Osteomyelitis:0.76), (RANOLAZINE:Osteomyelitis:0.77), (SECUKINUM AB:Osteomyelitis:0.39), (SOTALOL:Osteomyelitis:0.56), (TELMISARTAN:Osteomyelitis:0.35), (TERIFLUNOMIDE:Osteomyelitis:0.14), (THALIDOMIDE:Osteomyelitis:0.27), (TIOTROPIU M:Osteomyelitis:0.11), (TIZANIDINE HYDROCHLORIDE:Osteomyelitis:0.59), (TOFACITINIB CITRATE:Osteomyelitis:0.36), (TRIAMCINOLONE ACETONIDE:Osteomyelitis:0.47), (ZOLPIDEM TARTRATE:Osteomyelitis:0.49), (NAPROXEN:Osteonecrosis:0.03), (.ALPHA.1-PROTEINASE INHIBITOR HUMAN:Osteopenia:1.02), (ABACAVIR SULFATEDOLUTEGRAVIR SODIUMLAMIVUDINE:Osteopenia:0.34), (ACARBOSE:Osteopenia:0.42), (ACETAMINOPH ENOXYCODONEOXYCODONE HYDROCHLORIDE:Osteopenia:1.15), (ADO-TRASTUZUMAB EMTANSINE:Osteopenia:0.54), (AGALSIDASE BETA:Osteopenia:0.23), (ALBIGLUTIDE:Osteopenia:0.18), (ALCOHOL:Osteopenia:0.05), (ALI SKIREN HEMIFUMARATE:Osteopenia:0.53), (AMINO ACIDSDEXTROSEELECTROLYTES NOSSOYBEAN OILVITAMINS:Osteopenia:0.18), (AMIODARONE HYDROCHLORIDE:Osteopenia:1.03), (AMLODIPINE BESYLATEHYDROCHLOROTHIAZIDEOLMESARTAN MEDOXOMIL:Osteopenia:0.64), (AMLODIPINE BESYLATEHYDROCHLOROTHIAZIDEVALSARTAN:Osteopenia:0.4), (AMLODIPINEV ALSARTAN:Osteopenia:0.55), (AMMONIUM LACTATE:Osteopenia:0.5), (ASPIRINBUTALBITALCAFFEINE:Osteopenia:0.02), (ATROPI NEDIPHENOXYLATE:Osteopenia:0), (BACITRACIN:Osteopenia:0.36), (BETAMETHASON ECALCIPOTRIENE:Osteopenia:0.33), (BUMETANIDE:Osteopenia:1.58), (BUPRENORPHIN E HYDROCHLORIDE:Osteopenia:0.3), (BUPRENORPHINE HYDROCHLORIDENALOXONE HYDROCHLORIDE:Osteopenia:0.42), (CALCIUM ACETATE:Osteopenia:0.86), (CALCIUM\MAGNESIUM\ZINC:Osteopenia:0.75), (CANAGLIF LOZINMETFORMIN HYDROCHLORIDE:Osteopenia:0.54), (CANDESARTAN CILEXETILHYDROCHLOROTHIAZIDE:Osteopenia:1.38), (CEFDINIR:Osteopenia:0.06), (C HLORHEXIDINE:Osteopenia:0.43), (CHLORPHENIRAMINEHYDROCODONE:Osteopenia: 0.35), (CILASTATIN SODIUMIMIPENEM:Osteopenia:0.28), (CLIKSTAR:Osteopenia:0.09), (CLINDAMYCIN PHOSPHATE:Osteopenia:0.47), (CLOBAZAM:Osteopenia:0.2), (CORTICOTROPIN:Osteopeni a:0.75), (CYPROHEPTADINE HYDROCHLORIDE:Osteopenia:0.01), (DACLIZUMAB:Osteopenia:0.96), (DANAZOL:Osteop enia:0.44), (DASATINIB:Osteopenia:1.91), (DESONIDE:Osteopenia:0.9), (DESOXIMETASON E:Osteopenia:0.34), (DIFLUNISAL:Osteopenia:0.29), (DIMENHYDRINATE:Osteopenia:1.27), (DIPYRIDAMOLE:Osteopenia:0.11), (DOLASETRON MESYLATE:Osteopenia:0.06), (DORZOLAMIDE HYDROCHLORIDETIMOLOL MALEATE:Osteopenia:0.05), (DRONABINOL:Osteopenia:1.44), (DUTASTERIDETAMSULO SIN HYDROCHLORIDE:Osteopenia:0.41), (ECONAZOLE:Osteopenia:0.05), (EDOXABAN TOSYLATE:Osteopenia:0.53), (EMPAGLIFLOZINLINAGLIPTIN:Osteopenia:0.89), (EPLERE NONE:Osteopenia:0.49), (ERYTHROMYCIN:Osteopenia:0.99), (ESTRIOL:Osteopenia:0.11), (E THINYL ESTRADIOLNORGESTREL:Osteopenia:0.15), (FENOTEROL HYDROBROMIDE:Osteopenia:0.45), (FORMOTEROL FUMARATE\MOMETASONE FUROATE:Osteopenia:1.08), (GINGER:Osteopenia:0.06), (GLYCOPYRROLATE:Osteopenia:0.69), (GLYCOPYRROLATEINDACATEROL MALEATE:Osteopenia:0.14), (GLYCOPYRRONIUM:Osteopenia:0.55), (GOLD:Osteopenia:0.17), (GUANFACINE:Osteopenia:0.02), (GUANFACINE HYDROCHLORIDE:Osteopenia:0.03), (HYDROCODONE BITARTRATEIBUPROFEN:Osteopenia:0.55), (HYDROXYUREA:Osteopenia:0.94), (HYOSC YAMINE SULFATEMETHENAMINEMETHYLENE BLUEPHENYL SALICYLATE\SODIUM PHOSPHATE, MONOBASIC, MONOHYDRATE:Osteopenia:0.06), (ICOSAPENT ETHYL:Osteopenia:0.13), (ILOPROST:Osteopenia:0), (INDAPAMIDEPERINDOPRIL:Osteope nia:0.39), (INSULIN DEGLUDEC:Osteopenia:0.37), (INTERFERON:Osteopenia:0.2), (INTERFERON GAMMA-1B:Osteopenia:0.03), (LEVALBUTEROL:Osteopenia:0.04), (LIDOCAINE HYDROCHLORIDE:Osteopenia:1), (LOMITAPIDE MESYLATE:Osteopenia:0.82), (LURASIDONE HYDROCHLORIDE:Osteopenia:0.12), (MAGNESIUM GLUCONATE:Osteopenia:0.22), (MEFENAMIC ACID:Osteopenia:0.29), (MEPOLIZUMAB:Osteopenia:0.94), (MUPIROCINMUPIROCIN CALCIUM:Osteopenia:1.03), (NATEGLINIDE:Osteopenia:0.53), (NIRAPARIB:Osteopenia:0.05), (OCTREOTIDE:Osteopenia:0.27), (OLODATEROL HYDROCHLORIDETIOTROPIUM BROMIDE MONOHYDRATE:Osteopenia:1.03), (ORPHENADRINE:Osteopenia:0.19), (OXYBUTYNINO XYBUTYNIN CHLORIDE:Osteopenia:0.37), (PATIROMER:Osteopenia:0.26), (PEGINTERFERON BETA-1A:Osteopenia:1.2), (PEMETREXED DISODIUM:Osteopenia:0.34), (PENICILLIN G:Osteopenia:0.02), (PHENTERMINE HYDROCHLORIDE:Osteopenia:0.06), (PIMECROLIMUS:Osteopenia:0.44), (PIOGLITAZONE: Osteopenia:0.29), (PIRFENIDONE:Osteopenia:0.25), (PNEUMOCOCCAL VACCINE, POLYVALENT 23:Osteopenia:0.12), (PONATINIB:Osteopenia:0.95), (PRAZOSIN HYDROCHLORIDE:Osteopenia:0.41), (PREDNISOLONEPREDNISOLONE ACETATEPREDNISOLONE SODIUM SUCCINATE:Osteopenia:0.08), (PROTRIPTYLINE HYDROCHLORIDE:Osteopenia:0.66), (QUINACRINE:Osteopenia:0.22), (RIVASTIGMINE:Os teopenia:0.36), (SCOPOLAMINE:Osteopenia:1.05), (SILODOSIN:Osteopenia:0.45), (SILVER SULFADIAZINE:Osteopenia:0.09), (SITAGLIPTIN:Osteopenia:0.12), (SOFOSBUVIRVELPA TASVIR:Osteopenia:0.17), (SUNITINIB MALATE:Osteopenia:0.61), (TAMOXIFEN CITRATE:Osteopenia:0.39), (TERBUTALINE SULFATE:Osteopenia:1.01), (TETRACYCLINE:Osteopenia:1.28), (THALIDOMIDE:Osteopeni a:1.5), (THEOPHYLLINE:Osteopenia:0.36), (TRANDOLAPRIL:Osteopenia:0.29), (TRIPTOREL IN PAMOATE:Osteopenia:0.24), (VALGANCICLOVIR HYDROCHLORIDE:Osteopenia:0.43), (VINCRISTINE SULFATE:Osteopenia:0.14), (VISMODEGIB:Osteopenia:0.04), (YEAST:Osteopenia:0.12), (ZIL EUTON:Osteopenia:0.3), (.ALPHA.-TOCOPHEROLVITAMINS:Osteoporosis:0.46), (.BETA.-ACETYLDIGOXIN:Osteoporosis:0.21), (.BETA.-HYDROXYISOVALERIC ACIDARGININEGLUTAMINE:Osteoporosis:0.28), (ABIRATERONE:Osteoporosis:0.29), (A BOBOTULINUMTOXINA:Osteoporosis:1.01), (ACAMPROSATE:Osteoporosis:0.48), (ACETA MINOPHENAPRONALIDECAFFEINEPROPYPHENAZONE:Osteoporosis:1.39), (ACETA MINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Osteoporosis:0.68), (ACETAMINOPHENCHLORPHENIRAMINE MALEATEPSEUDOEPHEDRINE HYDROCHLORIDE:Osteoporosis:0.29), (ACETAMINOPHENCHLORZOXAZONE:Osteopor osis:0.11), (ACETAMINOPHENPSEUDOEPHEDRINE HYDROCHLORIDE:Osteoporosis:0.49), (ACETYLCYSTEINEBENZALKONIUM CHLORIDETUAMINOHEPTANE SULFATE:Osteoporosis:0.2), (ACLIDINIUM BROMIDEFORMOTEROL FUMARATE:Osteoporosis:0.24), (ACOTIAMIDE HYDROCHLORIDE:Osteoporosis:0.3), (ADENINE:Osteoporosis:1.62), (AGALSIDASE BETA:Osteoporosis:3.11), (ALDESLEUKIN:Osteoporosis:0), (ALECTINIB:Osteoporosis:0.04), (ALISKIREN HEMIFUMARATEVALSARTAN:Osteoporosis:0.33), (ALUMINUM HYDROXIDEMAGNESIUM OXIDEOXETHAZAINE:Osteoporosis:0.61), (AMIKACIN:Osteoporosis:0.31), (AMILORIDE HYDROCHLORIDE:Osteoporosis:0.01), (AMINO ACIDSVITAMINS:Osteoporosis:0.16), (AMINOSALICYLIC ACID:Osteoporosis:0.02), (AMMONIUM CHLORIDEDEXTROMETHORPHAN\SODIUM CITRATE:Osteoporosis:1), (AMOBARBITAL SODIUM:Osteoporosis:0.32), (ANHYDROUS CITRIC ACIDASPIRIN\SODIUM BICARBONATE:Osteoporosis:0.75), (APRINDINE HYDROCHLORIDE:Osteoporosis:1.38), (ARCTOSTAPHYLOS UVA-URSI LEAFDIOICA:Osteoporosis:0.14), (ARFORMOTEROL:Osteoporosis:0.17), (ARIPIPRAZOLE LAUROXIL:Osteoporosis:0.24), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Osteoporosis:1.35), (ASCORBIC ACIDBIOTINCYANOCOBALAMINFOLIC ACIDNIACINPANTOTHENIC ACIDPYRIDOXINE\RIBOFLAVINTHIAMINE:Osteoporosis:0.81), (ASPIRINBUTALBITA LCAFFEINECODEINE PHOSPHATE:Osteoporosis:0.22), (ASPIRIN\CAFFEINESALICYLAMIDE:Osteoporosis:0.55), (ASPIRINDIHYDROXYALUMINUM AMINOACETATEMAGNESIUM CARBONATE:Osteoporosis:0.25), (ATAZANAVIRRITONAVIR:Osteoporosis:0.21), (ATROPI NE SULFATE:Osteoporosis:1.2), (ATROPINE SULFATEDIPHENOXYLATE:Osteoporosis:0), (AVOBENZONEHOMOSALATEOCTISAL ATE:Osteoporosis:0.09), (AZITHROMYCIN:Osteoporosis:0.3), (AZITHROMYCIN OR METHOTREXATE SODIUM:Osteoporosis:1.66), (AZULENE:Osteoporosis:0.25), (BACLOFENCYCLOBENZAPR INEDICLOFENACGABAPENTINTETRACAINE:Osteoporosis:0.28), (BELATACEPT:Osteo porosis:0.36), (BEPRIDIL HYDROCHLORIDE:Osteoporosis:0.24), (BERBERINEGERANIUM:Osteoporosis:0.11), (BET AMETHASONE ACETATEBETAMETHASONE SODIUM PHOSPHATE:Osteoporosis:0.01), (BETAMETHASONE DIPROPIONATECALCIPOTRIENE:Osteoporosis:1.4), (BETAMETHASONE DIPROPIONATECALCIPOTRIENE HYDRATE:Osteoporosis:0.85), (BETAMETHASONECALCIPOTRIENE:Osteoporosis:1.86), (BETAMIPRONPANIPENEM:Osteoporosis:0.21), (BICARBONATE ION:Osteoporosis:0.95), (BICARBONATE IONPOLYETHYLENE GLYCOLSPOTASSIUM CATION\SODIUM CHLORIDE\SODIUM SULFATE ANHYDROUS:Osteoporosis:0.41), (LACTIS:Osteoporosis:0.63), (BIXALOMER:Osteoporosis:0.97), (BLINATUMOMAB:Osteopor osis:0.29), (BLONANSERIN:Osteoporosis:1.18), (BOSUTINIB:Osteoporosis:0.52), (BOSUTINI B MONOHYDRATE:Osteoporosis:0.3), (BRIMONIDINE TARTRATEDORZOLAMIDE HYDROCHLORIDETIMOLOL MALEATE:Osteoporosis:0.07), (BROMPERIDOL:Osteoporosis:0.16), (BUCOLOME:Osteoporo sis:0.46), (BUFORMIN:Osteoporosis:0.63), (BUPROPION HYDROBROMIDE:Osteoporosis:0.2), (BUTYLSCOPOLAMINE:Osteoporosis:0.29), (CABOZ ANTINIB:Osteoporosis:0.78), (CABOZANTINIB S-MALATE:Osteoporosis:0.75), (CAFFEINEISOMETHEPTENEMETHIMAZOLE:Osteoporosis: 0.58), (CAJUPUT OILEUCALYPTUS OILPEPPERMINT OIL:Osteoporosis:0.97), (CALCIUM ACETATEMAGNESIUM ACETATE\POTASSIUM ACETATE\SODIUM ACETATE\SODIUM CHLORIDE:Osteoporosis:0.24), (CALCIUM CHLORIDE\POTASSIUM CHLORIDE\SODIUM CHLORIDE\SODIUM LACTATESORBITOL:Osteoporosis:0.06), (CALCIUM PANTOTHENATECYANOCOBALAMINNIACINAMIDEPYRIDOXINE HYDROCHLORIDE\RIBOFLAVINTHIAMINE HYDROCHLORIDE:Osteoporosis:0.44), (CALCIUM PANTOTHENATECYANOCOBALAMINNIACINAMIDEPYRIDOXINE HYDROCHLORIDE\RIBOFLAVINTHIAMINE MONONITRATE:Osteoporosis:1.6), (CAMOSTAT:Osteoporosis:0.01), (CAMPHORCAPSICU M\METHYL SALICYLATE:Osteoporosis:0.38), (CARBAMIDE PEROXIDE:Osteoporosis:1.28), (CARBINOXAMINE MALEATE:Osteoporosis:0.06), (CARBOXYMETHYLCELLULOSE:Osteoporosis:0.11), (CAR PRONIUM CHLORIDE:Osteoporosis:0.82), (CARTEOLOL:Osteoporosis:0.35), (CEFOTAXIMECEFOTA XIME SODIUM:Osteoporosis:1.14), (CEFOZOPRAN HYDROCHLORIDE:Osteoporosis:0.36), (CEFTAZIDIME SODIUM:Osteoporosis:0.52), (CETUXIMAB:Osteoporosis:1.14), (CEVIMELINE:Osteoporosis: 0.51), (CHLORMADINONE ACETATEETHINYL ESTRADIOL:Osteoporosis:0.94), (CHLOROTHIAZIDE:Osteoporosis:1.2), (CHLORPROMAZI NEPROMETHAZINE:Osteoporosis:0.31), (CHOLINETRAZODONE HYDROCHLORIDE:Osteoporosis:0.25), (CICLOPIROX OLAMINE:Osteoporosis:0.53), (CIPROFLOXACINDEXAMETHASONE:Osteoporosis:0.29), (CITICOLINE:Osteoporosis:0.44), (CLENBUTEROL HYDROCHLORIDE:Osteoporosis:1.64), (CLOSTRIDIUM TETANICLOSTRIDIUM TETANI TOXOID ANTIGEN (FORMALDEHYDE INACTIVATED)DIPHTHERIAECORYNEBACTERIUM DIPHTHERIAE TOXOID ANTIGEN (FORMALDEHYDE INACTIVATED):Osteoporosis:0.09), (CLOXACILLIN SODIUM:Osteoporosis:0.43), (COAGULATION FACTOR IX RECOMBINANT HUMANISOPROPYL ALCOHOL:Osteoporosis:0.15), (COCONUT OIL:Osteoporosis:0.32), (CODEINE PHOSPHATEPROMETHAZINE:Osteoporosis:0.33), (CODEINE SULFATE:Osteoporosis:0.54), (COLESTILAN CHLORIDE:Osteoporosis:1.16), (CROTALIDAE POLYVALENT IMMUNE FAB (OVINE):Osteoporosis:0.42), (CYAMEMAZINE:Osteoporosis:1.14), (CYANAMIDE:Osteoporo sis:0.09), (CYANOCOBALAMINFOLIC ACIDIRON:Osteoporosis:0.17), (CYCLOPENTOLATE:Osteoporosis:0.73), (CYPROTERONE ACETATEETHINYL ESTRADIOL:Osteoporosis:0.52), (DABRAFENIB:Osteoporosis:1.06), (DACLIZUMAB:Osteop orosis:2.11), (DAPAGLIFLOZIN PROPANEDIOLMETFORMIN HYDROCHLORIDE:Osteoporosis:0.98), (DARIFENACIN:Osteoporosis:0.73), (DESFLURANE: Osteoporosis:0.31), (DEXAMETHASONENEOMYCINPOLYMYXIN B SULFATE:Osteoporosis:0.02), (DEXIBUPROFEN:Osteoporosis:0.23), (DEXTRAN:Osteoporosi s:0.32), (DEXTRAN SULFATE SODIUM SALT:Osteoporosis:0.38), (DEXTROMETHORPHAN HYDROBROMIDEQUINIDINE SULFATE:Osteoporosis:0.8), (DEXTROMETHORPHANDEXTROMETHORPHAN HYDROBROMIDEGUAIFENESIN:Osteoporosis:0.21), (DICHLORPHENAMIDE:Osteoporosi s:0.08), (DICLOFENAC DIETHYLAMINE:Osteoporosis:0.35), (DIENOGEST:Osteoporosis:0.14), (DIETARY SUPPLEMENTFLAVOCOXID:Osteoporosis:0.13), (DIFLUCORTOLONE VALERATELIDOCAINE:Osteoporosis:0.58), (DIHYDROERGOTAMINE MESYLATE:Osteoporosis:0.16), (DIMETHYL SULFOXIDE:Osteoporosis:0.29), (DIPHENHYDRAMINE HYDROCHLORIDELIDOCAINE HYDROCHLORIDENYSTATIN:Osteoporosis:0.79), (DIPHENHYDRAMINE HYDROCHLORIDEPHENYLEPHRINE HYDROCHLORIDE:Osteoporosis:0.17), (DOLASETRON MESYLATE:Osteoporosis:1.36), (DOXYCYCLINE HYDROCHLORIDE:Osteoporosis:0.69), (DOXYLAMINE SUCCINATEPYRIDOXINE HYDROCHLORIDE:Osteoporosis:0.37), (DUPILUMAB:Osteoporosis:0.03), (EDARAVONE:0 steoporosis:1.53), (ELBASVIRGRAZOPREVIR:Osteoporosis:0.49), (ELOSULFASE ALFA:Osteoporosis:0.49), (EMPAGLIFLOZINLINAGLIPTIN:Osteoporosis:1.31), (EMPAGLI FLOZINMETFORMIN HYDROCHLORIDE:Osteoporosis:0.01), (ENFUVIRTIDE:Osteoporosis:0.07), (EPHEDRA SINICA STEMHERBALS:Osteoporosis:0.75), (EPHEDRINE HYDROCHLORIDE:Osteoporosis:0.18), (EPRAZINONE:Osteoporosis:1.14), (ERGOLOID MESYLATES:Osteoporosis:0.8), (ERIBULIN:Osteoporosis:0.94), (ERYTHROMYCIN ETHYLSUCCINATE:Osteoporosis:0.14), (ERYTHROMYCINSULFISOXAZOLE:Osteoporosi s:1.2), (ESTRADIOL BENZOATE:Osteoporosis:0.82), (ESTROGENS, ESTERIFIED:Osteoporosis:0.04), (ESTROGENSMETHYLTESTOSTERONE:Osteoporosis:0.49), (ETHINYL ESTRADIOLETHYNODIOL DIACETATE:Osteoporosis:0.05), (ETHINYL ESTRADIOLFERROUS FUMARATENORETHINDRONE ACETATE:Osteoporosis:0.31), (ETHINYL ESTRADIOLGESTODENE:Osteoporosis:1.06), (ETHINYL ESTRADIOLNORGESTREL:Osteoporosis:1.38), (ETHOSUXIMIDE:Osteoporosis:0.4), (ETRE TINATE:Osteoporosis:3.09), (FELODIPINEMETOPROLOL SUCCINATE:Osteoporosis:0.13), (FERRIC POLYSACCHARIDE COMPLEX:Osteoporosis:0.53), (FERROUS BISGLYCINATE:Osteoporosis:1.78), (FINGOLIMOD:Osteoporosis:0.55), (FLAVOXATE HYDROCHLORIDE:Osteoporosis:1), (FLUOCINOLONE:Osteoporosis:0.45), (FLUOROURAC ILLEUCOVORIN CALCIUMOXALIPLATIN:Osteoporosis:0.3), (FLUOXETINE HYDROCHLORIDEOLANZAPINE:Osteoporosis:0.31), (FLUPHENAZINE HYDROCHLORIDE:Osteoporosis:0.01), (FLURANDRENOLIDE:Osteoporosis:0.38), (FOSFO MYCIN CALCIUM:Osteoporosis:1.25), (FOSFOMYCIN SODIUM:Osteoporosis:0.52), (FOSPHENYTOINFOSPHENYTOIN SODIUM:Osteoporosis:0.84), (FURSULTIAMINE:Osteoporosis:1.74), (FUSIDATE SODIUM:Osteoporosis:0.15), (GABAPENTIN ENACARBIL:Osteoporosis:0.51), (GADOTERATE MEGLUMINE:Osteoporosis:0.19), (GARLIC OIL:Osteoporosis:0.04), (GENTIAN VIOLET:Osteoporosis:0.35), (GLIMEPIRIDEROSIGLITAZONE MALEATE:Osteoporosis:0.38), (GLUCAGON HYDROCHLORIDE:Osteoporosis:0.03), (GLUCONATE:Osteoporosis:0.12), (GLUTAMINES ODIUM GUALENATE:Osteoporosis:0.76), (GOLD:Osteoporosis:0.76), (GUAIFENESINPHENYLEPH RINE HYDROCHLORIDE:Osteoporosis:0.37), (GUANABENZ ACETATE:Osteoporosis:1.75), (HALCINONIDE:Osteoporosis:0.39), (HALOPERIDOL DECANOATE:Osteoporosis:0.29), (HARPAGOPHYTUM PROCUMBENS ROOT:Osteoporosis:0.02), (HEPATITIS B VIRUS VACCINE:Osteoporosis:0.26), (HERBALSMINERALSVITAMINS:Osteoporosis:0.5), (HERB SROOTS:Osteoporosis:0.51), (HIGH DENSITY POLYETHYLENE:Osteoporosis:0.2), (HOMATROPINE METHYLBROMIDEHYDROCODONE BITARTRATE:Osteoporosis:0.33), (HYDRALAZINE HYDROCHLORIDEISOSORBIDE DINITRATE:Osteoporosis:0.4), (HYDRAZINE SULFATE:Osteoporosis:0.41), (HYDROCHLORIC ACID:Osteoporosis:0.14), (HYDROCHLOROTHIAZIDEMETOPROLOL TARTRATE:Osteoporosis:0.53), (HYDROXOCOBALAMIN ACETATE:Osteoporosis:0.39), (HYDROXYCITRIC ACID:Osteoporosis:0.57), (HYDROXYPROPYL CELLULOSE:Osteoporosis:0.31), (HYOSCYAMINE SULFATEMETHENAMINEMETHYLENE BLUEPHENYL SALICYLATE\SODIUM PHOSPHATE, MONOBASIC, MONOHYDRATE:Osteoporosis:1.15), (ICATIBANT ACETATE:Osteoporosis:0.3), (ILOPROST:Osteoporosis:1.37), (IMIPENEM:Osteoporosis:0.27), (INFLUENZA A VIRUS A/CALIFORNIA/7/2009 X-179A (HINI) ANTIGEN (FORMALDEHYDE INACTIVATED)\INFLUENZA A VIRUS A/VICTORIA/210/2009 X-187 (H3N2) ANTIGEN (FORMALDEHYDE INACTIVATED)\INFLUENZA B VIRUS B/BRISBANE/60/2008 ANTIGEN (FORMALDEHYDE INACTIVATED):Osteoporosis:0.32), (INFLUENZA VIRUS VACCINE TRIVALENT TYPE A+B AFLURIA:Osteoporosis:0.64), (INTERFERON ALFA-2A:Osteoporosis:1.6), (INTERFERON ALFA-2B:Osteoporosis:1.26), (INTERFERON BETA:Osteoporosis:0.6), (INTERFERON GAMMA-1B:Osteoporosis:0.82), (IPRAGLIFLOZIN:Osteoporosis:0.41), (IRBESARTANTRICHLORME THIAZIDE:Osteoporosis:0.23), (ISOFLAVONES SOY:Osteoporosis:0.16), (ISOFLURANE:Osteoporosis:0.12), (ISONIAZIDPYRAZINAMIDE RIFAMPIN:Osteoporosis:0.79), (DELBRUECKII SUBSP. BULGARICUS:Osteoporosis:1.45), (LANDIOLOL HYDROCHLORIDE:Osteoporosis:0.83), (LANOLINMINERAL OILPETROLATUM:Osteoporosis:0.11), (LARONIDASE:Osteoporosis:1.01), (LEVOSIMEND AN:Osteoporosis:0.5), (LINSEED OIL:Osteoporosis:0.25), (LINSIDOMINE:Osteoporosis:0.84), (LUMACAFTOR:Osteoporosis:0.51), (LUTETIUM LU-177:Osteoporosis:1.13), (MAGNESIUM PIDOLATE:Osteoporosis:0.43), (MECLIZINE MONOHYDROCHLORIDENIACIN:Osteoporosis:0.51), (MECLOZINE DIHYDROCHLORIDE:Osteoporosis:0.39), (MENTHOLZINC OXIDE:Osteoporosis:0.8), (MEQUITAZINE:Osteoporosis:0.69), (METFORMIN HYDROCHLORIDESAXAGLIPTIN:Osteoporosis:1.21), (METFORMIN PAMOATE:Osteoporosis:1.52), (METFORMINSITAGLIPTIN:Osteoporosis:0.74), (METHEN OLONE ACETATE:Osteoporosis:0.14), (METRELEPTIN:Osteoporosis:0.84), (METYRAPONE:Osteopo rosis:0.05), (MICAFUNGIN:Osteoporosis:0.21), (MIGLUSTAT:Osteoporosis:1.45), (MINERAL OILPETROLATUM:Osteoporosis:0.97), (MINERALSPROBIOTICS NOSVITAMINS:Osteoporosis:0.04), (MIRIPLATIN:Osteoporosis:0.03), (MORNIFLUMATE:0 steoporosis:0.26), (NABILONE:Osteoporosis:0.71), (NALBUPHINE HYDROCHLORIDE:Osteoporosis:0.63), (NALDEMEDINE:Osteoporosis:0.16), (NALOXONE: Osteoporosis:0.67), (NELARABINE:Osteoporosis:0.16), (NEOMYCIN:Osteoporosis:1.13), (NEO STIGMINE:Osteoporosis:0.83), (NIAOULI OILQUININE BENZOATETHIAMINE HYDROCHLORIDE:Osteoporosis:0.27), (NOMEGESTROL ACETATE:Osteoporosis:0.11), (NOREPINEPHRINE BITARTRATE:Osteoporosis:1.21), (NUSINERSEN:Osteoporosis:0.66), (OBETICHOLIC ACID:Osteoporosis:0.19), (OK-432:Osteoporosis:0.06), (OMEGA-3-CARBOXYLIC ACIDS:Osteoporosis:0.26), (OPIPRAMOL:Osteoporosis:2.35), (OSTREA EDULIS SHELL:Osteoporosis:0.08), (OXANDROLONE:Osteoporosis:0.09), (OXITRIPTAN:Osteoporosi s:0.38), (OZAGREL:Osteoporosis:0.08), (PALIPERIDONE PALMITATE:Osteoporosis:0.72), (PAPAYA:Osteoporosis:0.49), (PARSLEY:Osteoporosis:0.02), (PAZUFLOXACIN MESILATE:Osteoporosis:0.54), (PEGINTERFERON ALFA-2A OR PEGINTERFERON ALFA-2B:Osteoporosis:0.22), (PENICILLIN V BENZATHINE:Osteoporosis:0.15), (PENICILLIN V POTASSIUM:Osteoporosis:1.68), (PENTAZOCINE HYDROCHLORIDE:Osteoporosis:0.17), (PENTAZOCINE LACTATE:Osteoporosis:0.35), (PENTOSTATIN:Osteoporosis:0.18), (PEPPERMINT OIL:Osteoporosis:0.12), (PEPSIN:Osteoporosis:0.25), (PEROSPIRONE HYDROCHLORIDE:Osteoporosis:0.78), (PHENYLEPHRINE HYDROCHLORIDEPROMETHAZINE HYDROCHLORIDE:Osteoporosis:0.63), (PHENYLEPHRINE HYDROCHLORIDEWITCH HAZEL:Osteoporosis:0.68), (PHOSPHORUS:Osteoporosis:0.63), (PIRENZEPINE:Osteoporosis: 0.32), (PIRITRAMIDE:Osteoporosis:1.48), (POLYSTYRENE:Osteoporosis:1.48), (POLYSTYRE NE SULFONIC ACID:Osteoporosis:1.12), (PORFIMER SODIUM:Osteoporosis:0.12), (POTASSIUM BICARBONATE:Osteoporosis:0.06), (POTASSIUM BROMIDE:Osteoporosis:0.18), (POTASSIUM PHOSPHATE, MONOBASIC:Osteoporosis:0.87), (POVIDONE:Osteoporosis:0.58), (PRAMLINTIDE ACETATE:Osteoporosis:0.42), (PRAMOXINE HYDROCHLORIDE:Osteoporosis:0.82), (PREDNISOLONE ACETATESULFACETAMIDE SODIUM:Osteoporosis:0.63), (PREDNISOLONE SODIUM METAZOATE:Osteoporosis:0.03), (PREDNISOLONE SODIUM PHOSPHATE:Osteoporosis:1.23), (PREDNISONE ACETATE:Osteoporosis:0), (PRILOCAINE:Osteoporosis:0.07), (PRISTINAMYCIN:Osteoporos is:1.19), (PROCAINE:Osteoporosis:0.36), (PROCHLORPERAZINE EDISYLATE:Osteoporosis:0.46), (PROMAZINE:Osteoporosis:0.24), (PROMAZINE HYDROCHLORIDE:Osteoporosis:0.04), (PROPANTHELINE BROMIDE:Osteoporosis:0.17), (PROTRIPTYLINE HYDROCHLORIDE:Osteoporosis:2.15), (PYRIMETHAMINE:Osteoporosis:0.22), (RACECAD OTRIL:Osteoporosis:0.08), (RASPBERRY KETONE:Osteoporosis:0.03), (ROXATIDINE ACETATE HYDROCHLORIDE:Osteoporosis:0.45), (SAFINAMIDE MESYLATE:Osteoporosis:0.07), (SAPROPTERIN DIHYDROCHLORIDE:Osteoporosis:0.8), (SCOPOLAMINE HYDROBROMIDE:Osteoporosis:0.17), (SEA WATER:Osteoporosis:0.22), (SELEXIPAG:Osteoporosis:0.14), (SENNOSIDE A:Osteoporosis:0.01), (SENNOSIDE B:Osteoporosis:0.57), (SEVOFLURANE:Osteoporosis:0.32), (SHARK CARTILAGESHARK, UNSPECIFIED:Osteoporosis:0), (SIVELESTAT SODIUM:Osteoporosis:0.67), (SODIUM PHOSPHATE, DIBASIC:Osteoporosis:0.16), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Osteoporosis:0.15), (SOMATREM:Osteoporosis:0.27), (SPIRAMYCIN:Oste oporosis:0.06), (SPIRULINA:Osteoporosis:0.63), (STREPTOMYCIN SULFATE:Osteoporosis:0.13), (SULFACETAMIDE SODIUMSULFUR:Osteoporosis:0.42), (SULFAMERAZINETRIMETHOPRIM:Osteoporosis: 0.03), (SUPLATAST TOSILATE:Osteoporosis:2.65), (TABALUMAB:Osteoporosis:0.22), (TALIGLUCERASE ALFA:Osteoporosis:0.31), (TEGAFURURACIL:Osteoporosis:0.52), (TELOTRISTAT ETHYL:Osteoporosis:0.79), (TERBUTALINE:Osteoporosis:1.98), (TESAMORELIN:Osteoporos is:0.44), (TESTOSTERONE ENANTHATE:Osteoporosis:0.41), (THIOPENTAL:Osteoporosis:0.45), (THYMOL:Osteoporosis: 0.23), (TIARAMIDE:Osteoporosis:0.82), (TIGECYCLINE:Osteoporosis:0.09), (TINIDAZOLE: Osteoporosis:0.36), (TIOPRONIN:Osteoporosis:0.01), (TIPIRACIL HYDROCHLORIDETRIFLURIDINE:Osteoporosis:0.41), (TIROPRAMIDE:Osteoporosis:0.16), (TOLCAPONE:Osteoporosis:0.17), (TRABECTEDIN:Osteoporosis:0.25), (TRANILAST:Osteo porosis:0.56), (TREPROSTINIL DIOLAMINE:Osteoporosis:0.27), (TRYPTOPHAN:Osteoporosis:0.37), (TULOBUTEROL HYDROCHLORIDE:Osteoporosis:0.29), (VASOPRESSIN:Osteoporosis:0.47), (VINORELBINE VINORELBINE TARTRATE:Osteoporosis:0.09), (VORAPAXAR SULFATE:Osteoporosis:0.3), (ZINC CITRATE:Osteoporosis:0.34), (ZINC GLUCONATE:Osteoporosis:0.59), (ZINC SULFATE\ZINC SULFATE ANHYDROUS:Osteoporosis:0.75), (ZIPRASIDONE HYDROCHLORIDE:Osteoporosis:0.5), (ZIV-AFLIBERCEPT:Osteoporosis:0.32), (ACETAZOLAMIDE:Osteoporosis postmenopausal:0.95), (ACLIDINIUM BROMIDE:Osteoporosis postmenopausal:0.46), (AGALSIDASE BETA:Osteoporosis postmenopausal:0.36), (ALISKIREN HEMIFUMARATE:Osteoporosis postmenopausal:0.3), (AMANTADINE HYDROCHLORIDE:Osteoporosis postmenopausal:0.28), (AMLODIPINE BESYLATEHYDROCHLOROTHIAZIDEOLMESARTAN MEDOXOMIL:Osteoporosis postmenopausal:0.14), (AMLODIPINE BESYLATEOLMESARTAN MEDOXOMIL:Osteoporosis postmenopausal:0.14), (AMLODIPINEVALSARTAN:Osteoporosis postmenopausal:0.08), (ARMODAFINIL:Osteoporosis postmenopausal:1.51), (AZELASTINE:Osteoporosis postmenopausal:0.33), (BETAMETHASONE DIPROPIONATECALCIPOTRIENECALCIPOTRIENE HYDRATE:Osteoporosis postmenopausal:0.04), (BETAMETHASONECALCIPOTRIENE:Osteoporosis postmenopausal:0.2), (BROTIZOLAM:Osteoporosis postmenopausal:0.14), (BUDESONIDEFORMOTEROL FUMARATE:Osteoporosis postmenopausal:0.9), (BUPRENORPHINE HYDROCHLORIDE:Osteoporosis postmenopausal:0.42), (BUPRENORPHINE HYDROCHLORIDENALOXONE HYDROCHLORIDE:Osteoporosis postmenopausal:0.41), (CALCIPOTRIENE:Osteoporosis postmenopausal:0.1), (CALCIUM\MAGNESIUM:Osteoporosis postmenopausal:0.32), (CALCIUM\MAGNESIUM\ZINC:Osteoporosis postmenopausal:0.3), (CANAGLIFLOZINMETFORMIN HYDROCHLORIDE:Osteoporosis postmenopausal:0.36), (CANAKINUMAB:Osteoporosis postmenopausal:0.05), (CARFILZOMIB:Osteoporosis postmenopausal:0.38), (CARVEDILOL PHOSPHATE:Osteoporosis postmenopausal:1.29), (CEFTRIAXONE SODIUM:Osteoporosis postmenopausal:0.46), (CETIRIZINE HYDROCHLORIDEPSEUDOEPHEDRINE HYDROCHLORIDE:Osteoporosis postmenopausal:0.21), (CLOBAZAM:Osteoporosis postmenopausal:0.03), (CORTICOTROPIN:Osteoporosis postmenopausal:0.47), (DABIGATRAN:Osteoporosis postmenopausal:0.22), (DACLIZUMAB:Osteoporosis postmenopausal:0.71), (DANAZOL:Osteoporosis postmenopausal:0.23), (DASATINIB:Osteoporosis postmenopausal:0.75), (DEFLAZACORT:Osteoporosis postmenopausal:1.09), (DESONIDE:Osteoporosis postmenopausal:0.19), (DEVICE:Osteoporosis postmenopausal:0.45), (DIPYRONE:Osteoporosis postmenopausal:0.1), (DOFETILIDE:Osteoporosis postmenopausal:0.73), (DOXAZOSIN MESYLATE:Osteoporosis postmenopausal:0.83), (DOXEPIN HYDROCHLORIDE:Osteoporosis postmenopausal:0.67), (DUTASTERIDE:Osteoporosis postmenopausal:1.01), (EMPAGLIFLOZINLINAGLIPTIN:Osteoporosis postmenopausal:0.72), (FENOFIBRIC ACID:Osteoporosis postmenopausal:0.51), (FORMOTEROL FUMARATE:Osteoporosis postmenopausal:0.99), (HALOPERIDOL:Osteoporosis postmenopausal:0.35), (IBRUTINIB:Osteoporosis postmenopausal:0.58), (ILOPROST:Osteoporosis postmenopausal:0.29), (IMATINIB MESYLATE:Osteoporosis postmenopausal:0.36), (INDACATEROL:Osteoporosis postmenopausal:0.31), (INDAPAMIDEPERINDOPRIL:Osteoporosis postmenopausal:0.85), (INDOMETHACIN:Osteoporosis postmenopausal:0.66), (INSULIN BEEF:Osteoporosis postmenopausal:0.18), (INSULIN DEGLUDEC:Osteoporosis postmenopausal:0.17), (INSULIN GLULISINE:Osteoporosis postmenopausal:0.36), (KETOROLACKETOROLAC TROMETHAMINE:Osteoporosis postmenopausal:0.13), (LACTASE:Osteoporosis postmenopausal:0.21), (LENALIDOMIDE:Osteoporosis postmenopausal:0.34), (LERCANIDIPINE:Osteoporosis postmenopausal:1.23), (LISDEXAMFETAMINE DIMESYLATE:Osteoporosis postmenopausal:0.59), (LOMITAPIDE MESYLATE:Osteoporosis postmenopausal:0.41), (MEGESTROL ACETATE:Osteoporosis postmenopausal:0.03), (MEPOLIZUMAB:Osteoporosis postmenopausal:0.63), (METFORMIN HYDROCHLORIDEVILDAGLIPTIN:Osteoporosis postmenopausal:0.04), (METHYLPREDNISOLONE SODIUM SUCCINATE:Osteoporosis postmenopausal:0.97), (MIDODRINE HYDROCHLORIDE:Osteoporosis postmenopausal:0.01), (MINOCYCLINE HYDROCHLORIDE:Osteoporosis postmenopausal:0.08), (NILOTINIB:Osteoporosis postmenopausal:0.97), (NINTEDANIB:Osteoporosis postmenopausal:0.17), (NORETHINDRONE:Osteoporosis postmenopausal:0.16), (OLODATEROL HYDROCHLORIDETIOTROPIUM BROMIDE MONOHYDRATE:Osteoporosis postmenopausal:0.3), (ORPHENADRINE:Osteoporosis postmenopausal:0.15), (OXYBUTYNINOXYBUTYNIN CHLORIDE:Osteoporosis postmenopausal:0.04), (PATIROMER:Osteoporosis postmenopausal:0.11), (PAZOPANIB HYDROCHLORIDE:Osteoporosis postmenopausal:0.45), (PEGINTERFERON BETA-1A:Osteoporosis postmenopausal:0.76), (PHENYTOIN SODIUM:Osteoporosis postmenopausal:0.28), (PIROXICAM:Osteoporosis postmenopausal:0.39), (PONATINIB:Osteoporosis postmenopausal:0.22), (PREDNISOLONE ACETATE:Osteoporosis postmenopausal:0.46), (PRIMIDONE:Osteoporosis postmenopausal:1.49), (RIZATRIPTAN:Osteoporosis postmenopausal:0.22), (ROFECOXIB:Osteoporosis postmenopausal:0.04), (SACUBITRILVALSARTAN:Osteoporosis postmenopausal:0.39), (SALMETEROL XINAFOATE:Osteoporosis postmenopausal:0.28), (SECUKINUMAB:Osteoporosis postmenopausal:0.86), (SILODOSIN:Osteoporosis postmenopausal:0.06), (SULFAMETHOXAZOLE:Osteoporosis postmenopausal:0.15), (SULINDAC:Osteoporosis postmenopausal:0.03), (SUNITINIB MALATE:Osteoporosis postmenopausal:0.3), (TAMSULOSIN:Osteoporosis postmenopausal:2.13), (TERAZOSINTERAZOSIN HYDROCHLORIDE:Osteoporosis postmenopausal:0.11), (TERIFLUNOMIDE:Osteoporosis postmenopausal:0.99), (ZAFIRLUKAST:Osteoporosis postmenopausal:0.36), (.ALPHA.1-PROTEINASE INHIBITOR HUMAN:Osteoporosis prophylaxis:0.05), (ACETAMINOPHENOXYCODONE HYDROCHLORIDEOXYCODONE TEREPHTHALATE:Osteoporosis prophylaxis:0.02), (ACETAMINOPHENOXYCODONEOXYCODONE HYDROCHLORIDE:Osteoporosis prophylaxis:0.47), (ACETAZOLAMIDE:Osteoporosis prophylaxis:1.22), (ACTIVATED CHARCOAL:Osteoporosis prophylaxis:0.29), (AGALSIDASE BETA:Osteoporosis prophylaxis:0.09), (ALEMTUZUMAB:Osteoporosis prophylaxis:0.58), (ALFUZOSIN:Osteoporosis prophylaxis:0.36), (ALUMINUM HYDROXIDEDIMETHICONEMAGNESIUM HYDROXIDE:Osteoporosis prophylaxis:0.32), (ALUMINUM HYDROXIDEMAGNESIUM CARBONATE:Osteoporosis prophylaxis:0.22), (AMIKACIN SULFATE:Osteoporosis prophylaxis:0.19), (AMILORIDE HYDROCHLORIDEHYDROCHLOROTHIAZIDE:Osteoporosis prophylaxis:0.39), (AMISULPRIDE:Osteoporosis prophylaxis:0.28), (AMLODIPINE BESYLATEATORVASTATIN CALCIUM:Osteoporosis prophylaxis:0.2), (AMLODIPINE BESYLATEHYDROCHLOROTHIAZIDEVALSARTAN:Osteoporosis prophylaxis:0.14), (AMOXAPINE:Osteoporosis prophylaxis:0.12), (AMPHETAMINE ASPARTATEAMPHETAMINE SULFATEDEXTROAMPHETAMINE SACCHARATEDEXTROAMPHETAMINE SULFATE:Osteoporosis prophylaxis:0.83), (ANAKINRA:Osteoporosis prophylaxis:0.38), (ATROPINE SULFATEDIPHENOXYLATE HYDROCHLORIDE:Osteoporosis prophylaxis:0.08), (AZELASTINE:Osteoporosis prophylaxis:0.04), (AZILSARTAN KAMEDOXOMIL:Osteoporosis prophylaxis:1.31), (BENAZEPRILBENAZEPRIL HYDROCHLORIDE:Osteoporosis prophylaxis:0.64), (BEPRIDIL:Osteoporosis prophylaxis:0.1), (BETAMETHASONE DIPROPIONATECALCIPOTRIENECALCIPOTRIENE HYDRATE:Osteoporosis prophylaxis:0.07), (BLINATUMOMAB:Osteoporosis prophylaxis:0.25), (BUDESONIDEFORMOTEROL FUMARATE:Osteoporosis prophylaxis:0.78), (BUPRENORPHINE HYDROCHLORIDENALOXONE HYDROCHLORIDE:Osteoporosis prophylaxis:0.48), (CALCITONIN:Osteoporosis prophylaxis:0.22), (CALCIUM ACETATE:Osteoporosis prophylaxis:0.27), (CALCIUM\MAGNESIUM:Osteoporosis prophylaxis:0.15), (CANAGLIFLOZINMETFORMIN HYDROCHLORIDE:Osteoporosis prophylaxis:0.31), (CERITINIB:Osteoporosis prophylaxis:0.04), (CETUXIMAB:Osteoporosis prophylaxis:0.81), (CHLORHEXIDINE:Osteoporosis prophylaxis:0.24), (CILASTATIN SODIUMIMIPENEM:Osteoporosis prophylaxis:1.13), (CLONIDINE HYDROCHLORIDE:Osteoporosis prophylaxis:0.07), (CORTICOTROPIN:Osteoporosis prophylaxis:0.21), (DACLIZUMAB:Osteoporosis prophylaxis:1.02), (DANAZOL:Osteoporosis prophylaxis:0.82), (DAPAGLIFLOZIN PROPANEDIOL:Osteoporosis prophylaxis:0.28), (DASATINIB:Osteoporosis prophylaxis:1.77), (DESONIDE:Osteoporosis prophylaxis:0.24), (DEVICE:Osteoporosis prophylaxis:0.27), (DOFETILIDE:Osteoporosis prophylaxis:0.47), (DOLUTEGRAVIR:Osteoporosis prophylaxis:0.4), (DOXEPIN HYDROCHLORIDE:Osteoporosis prophylaxis:0.32), (DOXYCYCLINE HYCLATE:Osteoporosis prophylaxis:0), (DRONABINOL:Osteoporosis prophylaxis:0.88), (DUTASTERIDETAMSULOSIN HYDROCHLORIDE:Osteoporosis prophylaxis:0.14), (ECONAZOLE:Osteoporosis prophylaxis:1), (EDOXABAN TOSYLATE:Osteoporosis prophylaxis:1.71), (ELETRIPTAN HYDROBROMIDE:Osteoporosis prophylaxis:1.15), (ELOTUZUMAB:Osteoporosis prophylaxis:0.3), (EMPAGLIFLOZINLINAGLIPTIN:Osteoporosis prophylaxis:0.2), (EPERISONE HYDROCHLORIDE:Osteoporosis prophylaxis:0), (EPIRUBICIN:Osteoporosis prophylaxis:0.5), (ERIBULIN:Osteoporosis prophylaxis:0.02), (ERYTHROMYCIN:Osteoporosis prophylaxis:0.77), (ETRETINATE:Osteoporosis prophylaxis:0.88), (FENOFIBRIC ACID:Osteoporosis prophylaxis:0.31), (FENOTEROL HYDROBROMIDE:Osteoporosis prophylaxis:0.26), (FERROUS SULFATEGLYCINE:Osteoporosis prophylaxis:0.35), (FESOTERODINE FUMARATE:Osteoporosis prophylaxis:1.08), (FEXOFENADINEPSEUDOEPHEDRINE:Osteoporosis prophylaxis:0.02), (FLAVIN ADENINE DINUCLEOTIDE:Osteoporosis prophylaxis:0.04), (FLUDROCORTISONE ACETATE:Osteoporosis prophylaxis:0.54), (FLUTICASONE PROPIONATEFORMOTEROL FUMARATE:Osteoporosis prophylaxis:0.19), (FORMOTEROL FUMARATE DIHYDRATE\MOMETASONE FUROATE:Osteoporosis prophylaxis:0.75), (FORMOTEROL FUMARATE\MOMETASONE FUROATE:Osteoporosis prophylaxis:0.26), (GLYCOPYRRONIUM:Osteoporosis prophylaxis:0.02), (HERBALSSAW PALMETTO:Osteoporosis prophylaxis:0.13), (HYDROCORTISONE ACETATE:Osteoporosis prophylaxis:0.55), (IBRITUMOMAB TIUXETAN:Osteoporosis prophylaxis:0.56), (ILOPROST:Osteoporosis prophylaxis:0.85), (IMIPRAMINE HYDROCHLORIDE:Osteoporosis prophylaxis:0.65), (INFLIXIMAB-DYYB:Osteoporosis prophylaxis:0.59), (INSULIN BEEF:Osteoporosis prophylaxis:0.1), (INSULIN DEGLUDEC:Osteoporosis prophylaxis:0.07), (INTERFERON:Osteoporosis prophylaxis:0.09), (INTERFERON ALFA-2A:Osteoporosis prophylaxis:0.27), (LABETALOLLABETALOL HYDROCHLORIDE:Osteoporosis prophylaxis:1.12), (LACTOBACILLUS ACIDOPHILUS:Osteoporosis prophylaxis:0.06), (LANREOTIDE ACETATE:Osteoporosis prophylaxis:0.78), (LAPATINIB DITOSYLATE:Osteoporosis prophylaxis:0.27), (LEVOLEUCOVORIN CALCIUM:Osteoporosis prophylaxis:0.8), (LIDOCAINEPRILOCAINE:Osteoporosis prophylaxis:0.53), (MECLIZINE HYDROCHLORIDE:Osteoporosis prophylaxis:0.38), (MECLIZINEMECLIZINE HYDROCHLORIDE:Osteoporosis prophylaxis:0.07), (MEPERIDINE:Osteoporosis prophylaxis:0.1), (MEPERIDINE HYDROCHLORIDE:Osteoporosis prophylaxis:0.45), (MEPOLIZUMAB:Osteoporosis prophylaxis:0.12), (MESNA:Osteoporosis prophylaxis:0.55), (METFORMIN HYDROCHLORIDEVILDAGLIPTIN:Osteoporosis prophylaxis:0.56), (METHENOLONE ENANTHATE:Osteoporosis prophylaxis:0.6), (METHYLPHENIDATE HYDROCHLORIDE:Osteoporosis prophylaxis:0.48), (MILK THISTLE:Osteoporosis prophylaxis:0.03), (MINOCYCLINE HYDROCHLORIDE:Osteoporosis prophylaxis:1.08), (MUPIROCIN:Osteoporosis prophylaxis:0.39), (MUPIROCINMUPIROCIN CALCIUM:Osteoporosis prophylaxis:0.48), (NABUMETONE:Osteoporosis prophylaxis:0.62), (NICARDIPINE:Osteoporosis prophylaxis:0.3), (NICARDIPINE HYDROCHLORIDE:Osteoporosis prophylaxis:0.3), (NIRAPARIB:Osteoporosis prophylaxis:0.12), (NITROFURANTOINNITROFURANTOIN MONOHYDRATE:Osteoporosis prophylaxis:0.26), (OCTREOTIDE:Osteoporosis prophylaxis:0.56), (OLAPARIB:Osteoporosis prophylaxis:0.28), (OLODATEROL HYDROCHLORIDETIOTROPIUM BROMIDE MONOHYDRATE:Osteoporosis prophylaxis:0.9), (OXCARBAZEPINE:Osteoporosis prophylaxis:2.29), (PAROXETINE:Osteoporosis prophylaxis:0.99), (PAZOPANIB HYDROCHLORIDE:Osteoporosis prophylaxis:1.11), (PEGINTERFERON BETA-1A:Osteoporosis prophylaxis:0.5), (PENICILLIN G:Osteoporosis prophylaxis:0.33), (PHLOROGLUCINOL1,3,5-TRIMETHOXYBENZENE:Osteoporosis prophylaxis:0.07), (PILOCARPINE:Osteoporosis prophylaxis:0.03), (PILSICAINIDE HYDROCHLORIDE:Osteoporosis prophylaxis:0.44), (PIMOBENDAN:Osteoporosis prophylaxis:0.29), (PREDNISOLONE ACETATE:Osteoporosis prophylaxis:0.73), (PRIMIDONE:Osteoporosis prophylaxis:1.12), (PSYLLIUM HUSK:Osteoporosis prophylaxis:0.19), (PYRAZINAMIDE:Osteoporosis prophylaxis:0.74), (QUINACRINE:Osteoporosis prophylaxis:0), (QUINAPRIL:Osteoporosis prophylaxis:0.09), (RADIATION THERAPY:Osteoporosis prophylaxis:0.05), (RAMUCIRUMAB:Osteoporosis prophylaxis:0.41), (RETINOL:Osteoporosis prophylaxis:0.01), (RILMENIDINE PHOSPHATE:Osteoporosis prophylaxis:0.15), (RIVASTIGMINE:Osteoporosis prophylaxis:0.33), (RIZATRIPTAN:Osteoporosis prophylaxis:0.03), (ROFLUMILAST:Osteoporosis prophylaxis:0.95), (SALMETEROL:Osteoporosis prophylaxis:0.29), (SELEXIPAG:Osteoporosis prophylaxis:0.51), (SODIUM FLUORIDE:Osteoporosis prophylaxis:0.18), (SUMATRIPTAN:Osteoporosis prophylaxis:0.29), (SUMATRIPTAN SUCCINATE:Osteoporosis prophylaxis:0.18), (SUPLATAST TOSILATE:Osteoporosis prophylaxis:0.15), (TERIFLUNOMIDE:Osteoporosis prophylaxis:0.46), (TETRACYCLINE:Osteoporosis prophylaxis:0.57), (TRANDOLAPRIL:Osteoporosis prophylaxis:0.03), (TRIPTORELIN PAMOATE:Osteoporosis prophylaxis:0.04), (UMECLIDINIUM BROMIDEVILANTEROL TRIFENATATE:Osteoporosis prophylaxis:0.28), (VEMURAFENIB:Osteoporosis prophylaxis:0.4), (VINORELBINE TARTRATE:Osteoporosis prophylaxis:0.21), (ZINC:Osteoporosis prophylaxis:0.43), (AMIODARONE:Osteoporotic fracture:0.11), (BUPROPION HYDROCHLORIDE:Osteoporotic fracture:0.09), (EVOLOCUMAB:Osteoporotic fracture:0.21), (IBRUTINIB:Osteoporotic fracture:0.07), (MELOXICAM:Osteoporotic fracture:0.14), (BUPROPION HYDROCHLORIDE:Otitis media:0.1), (CALCIUM CARBONATECHOLECALCIFEROL:Otitis media:0.06), (EVOLOCUMAB:Otitis media:0.06), (MELOXICAM:Otitis media:0.09), (RABEPRAZOLE SODIUM:Otitis media:0.09), (SPIRONOLACTONE:Otitis media:0.26), (ACARBOSE:Ovarian cancer:0.29), (ACETAMINOPHENOXYCODONEOXYCODONE HYDROCHLORIDE:Ovarian cancer:0.46), (ACETAMINOPHENPROPOXYPHENE NAPSYLATE:Ovarian cancer:0.04), (ACETAZOLAMIDE:Ovarian cancer:0.36), (ALCOHOL:Ovarian cancer:0.09), (ALFUZOSIN:Ovarian cancer:0.1), (ALFUZOSIN HYDROCHLORIDE:Ovarian cancer:0.06), (ALISKIREN HEMIFUMARATE:Ovarian cancer:0.06), (AMANTADINE:Ovarian cancer:0.3), (AMANTADINE HYDROCHLORIDE:Ovarian cancer:0.04), (AMINO ACIDSDEXTROSEELECTROLYTES NOS:Ovarian cancer:0.04), (AMISULPRIDE:Ovarian cancer:0.5), (AMLODIPINE BESYLATEHYDROCHLOROTHIAZIDEOLMESARTAN MEDOXOMIL:Ovarian cancer:0.32), (ARMODAFINIL:Ovarian cancer:0.81), (ATROPINEDIPHENOXYLATE:Ovarian cancer:0.1), (AXITINIB:Ovarian cancer:0.5), (BACITRACIN:Ovarian cancer:0.04), (BENAZEPRILBENAZEPRIL HYDROCHLORIDE:Ovarian cancer:0.49), (BUDESONIDEFORMOTEROL FUMARATE:Ovarian cancer:0), (BUPRENORPHINE HYDROCHLORIDE:Ovarian cancer:0.3), (BUPRENORPHINE HYDROCHLORIDENALOXONE HYDROCHLORIDE:Ovarian cancer:0.23), (CALCITONIN:Ovarian cancer:0.06), (CALCITONIN SALMON:Ovarian cancer:0.91), (CANAGLIFLOZINMETFORMIN HYDROCHLORIDE:Ovarian cancer:0.17), (CAPTOPRIL:Ovarian cancer:0.4), (CARBIMAZOLE:Ovarian cancer:0.09), (CARFILZOMIB:Ovarian cancer:1.09), (CEFUROXIME AXETIL:Ovarian cancer:0.02), (CERTOLIZUMAB PEGOL:Ovarian cancer:1.46), (CHLORDIAZEPOXIDE HYDROCHLORIDECLIDINIUM BROMIDE:Ovarian cancer:0.4), (CLINDAMYCIN PHOSPHATE:Ovarian cancer:0.18), (CLOTRIMAZOLE:Ovarian cancer:1.06), (DABIGATRAN:Ovarian cancer:0.12), (DABRAFENIB:Ovarian cancer:1.05), (DABRAFENIB MESYLATE:Ovarian cancer:0.34), (DANAZOL:Ovarian cancer:0.32), (DEVICE:Ovarian cancer:0.07), (DEXAMETHASONE ACETATE:Ovarian cancer:0.2), (DIPYRONE:Ovarian cancer:0.29), (DOFETILIDE:Ovarian cancer:0.61), (DOXAZOSIN MESYLATE:Ovarian cancer:0.74), (DOXEPIN HYDROCHLORIDE:Ovarian cancer:0.1), (DUTASTERIDE:Ovarian cancer:1.18), (ECONAZOLE:Ovarian cancer:0.25), (EPIRUBICIN HYDROCHLORIDE:Ovarian cancer:0.11), (ESTROGENS, CONJUGATEDMEDROXYPROGESTERONE ACETATE:Ovarian cancer:0.43), (EVOLOCUMAB:Ovarian cancer:0.35), (FENOFIBRIC ACID:Ovarian cancer:0.19), (FERROUS FUMARATE:Ovarian cancer:0.27), (FESOTERODINE FUMARATE:Ovarian cancer:0.86), (FEXOFENADINEPSEUDOEPHEDRINE:Ovarian cancer:0.15), (FLUDROCORTISONE ACETATE:Ovarian cancer:0.44), (FLUTICASONE FUROATE:Ovarian cancer:0.31), (FONDAPARINUX:Ovarian cancer:0.06), (FONDAPARINUX SODIUM:Ovarian cancer:0.15), (FORMOTEROL:Ovarian cancer:0.1), (FORMOTEROL FUMARATE:Ovarian cancer:0.28), (GEFITINIB:Ovarian cancer:0.46), (GEMFIBROZIL:Ovarian cancer:0.39), (GLYCOPYRRONIUMlNDACATEROL:Ovarian cancer:0.51), (HERBALSSAW PALMETTO:Ovarian cancer:0.01), (HYALURONATE SODIUM:Ovarian cancer:0.1), (HYDROXYCHLOROQUINE DIPHOSPHATE:Ovarian cancer:0.13), (HYDROXYUREA:Ovarian cancer:0.28), (IBRUTINIB:Ovarian cancer:0.89), (IDELALISIB:Ovarian cancer:0.03), (IMIPRAMINE HYDROCHLORIDE:Ovarian cancer:0.28), (INFLIXIMAB-DYYB:Ovarian cancer:0.18), (INFLUENZA VIRUS VACCINE:Ovarian cancer:0.42), (INTERFERON ALFA-2A:Ovarian cancer:0.44), (ISOSORBIDE:Ovarian cancer:0.71), (LABETALOL HYDROCHLORIDE:Ovarian cancer:0.2), (LAPATINIB:Ovarian cancer:0.05), (MECLIZINEMECLIZINE HYDROCHLORIDE:Ovarian cancer:0.5), (MEROPENEM:Ovarian cancer:0.27), (METAXALONE:Ovarian cancer:1.17), (METFORMIN HYDROCHLORIDEVILDAGLIPTIN:Ovarian cancer:0.29), (METHENAMINE HIPPURATE:Ovarian cancer:0.39), (METHYLPREDNISOLONE ACETATE:Ovarian cancer:0.88), (MIDODRINE:Ovarian cancer:0.4), (NABUMETONE:Ovarian cancer:0.37), (NADOLOL:Ovarian cancer:0.47), (NICOTINE:Ovarian cancer:0.3), (NILOTINIB:Ovarian cancer:1.88), (OLMESARTAN:Ovarian cancer:0.02), (OLODATEROL HYDROCHLORIDETIOTROPIUM BROMIDE MONOHYDRATE:Ovarian cancer:0.33), (OMEPRAZOLE SODIUM:Ovarian cancer:0.42), (OXYBUTYNIN:Ovarian cancer:0.21), (PANTOPRAZOLE MAGNESIUM:Ovarian cancer:1.16), (PENICILLIN G:Ovarian cancer:0.07), (PHENOBARBITAL:Ovarian cancer:0.65), (PHENYTOIN SODIUM:Ovarian cancer:0.04), (PITAVASTATIN CALCIUM:Ovarian cancer:0.75), (POMALIDOMIDE:Ovarian cancer:0.03), (PRIMIDONE:Ovarian cancer:1.08), (PSYLLIUM HUSK:Ovarian cancer:0.23), (RADIATION THERAPY:Ovarian cancer:1.55), (RAMUCIRUMAB:Ovarian cancer:1.05), (RASBURICASE:Ovarian cancer:0.21), (RIVASTIGMINE:Ovarian cancer:0.08), (RIVASTIGMINE TARTRATE:Ovarian cancer:0.2), (ROFLUMILAST:Ovarian cancer:0.6), (SACUBITRILVALSARTAN:Ovarian cancer:0.38), (SECUKINUMAB:Ovarian cancer:0.14), (SODIUM OXYBATE:Ovarian cancer:0.7), (SULFAMETHOXAZOLE:Ovarian cancer:0.57), (TELOTRISTAT ETHYL:Ovarian cancer:0.05), (TERBINAFINE HYDROCHLORIDE:Ovarian cancer:0.47), (TERIFLUNOMIDE:Ovarian cancer:0.09), (TIMOLOL:Ovarian cancer:1.16), (TINZAPARIN:Ovarian cancer:0.35), (TIOTROPIUM:Ovarian cancer:0.61), (TIZANIDINE:Ovarian cancer:0.66), (TOFACITINIB CITRATE:Ovarian cancer:0.01), (TROSPIUM CHLORIDE:Ovarian cancer:0.06), (UMECLIDINIUM BROMIDEVILANTEROL TRIFENATATE:Ovarian cancer:0.44), (VILAZODONE HYDROCHLORIDE:Ovarian cancer:0.1), (ZIV-AFLIBERCEPT:Ovarian cancer:0.1), (AMPHETAMINE ASPARTATEAMPHETAMINE SULFATEDEXTROAMPHETAMINE SACCHARATEDEXTROAMPHETAMINE SULFATE:Ovarian cyst:0.21), (MENOTROPINS:Ovarian hyperstimulation syndrome:0.12), (LITHIUM CARBONATE:Overdose:0.02), (NAPROXEN:Overdose:0.02), (VARENICLINE TARTRATE:Overdose:0.09), (ZONISAMIDE:Overdose:0.02), (SODIUM OXYBATE:Overweight:0.07), (FOLLICLE STIMULATING HORMONE BETA POLYPEPTIDELUTROPIN ALFA:Ovulation disorder:0.66), (MENOTROPINS:Ovulation disorder:0.07), (.ALPHA.-TOCOPHEROL ACETATE, D-ASCORBIC ACIDCALCIUM PANTOTHENATEFOLIC ACIDMANGANESENIACINAMIDEPYRIDOXINE\RIBOFLAVINTHIAMINEVITAMIN A\ZINC OXIDE:Pain:1.09), (.ALPHA.-TOCOPHEROLASCORBIC ACID:Pain:0.23), (.ALPHA.-TOCOPHEROLASCORBIC ACIDBIOTINCALCIUM CARBONATECHOLECALCIFEROLCOPPERCYANOCOBALAMINFERROUS FUMARATEFOLIC ACIDIODINEMAGNESIUMNIACINAMIDEPANTOTHENIC ACIDPYRIDOXINE\RIBOFLAVINTHIAMINEVITAMIN A\ZINC:Pain:0.45), (.BETA.-HYDROXYISOVALERIC ACIDARGININEGLUTAMINE:Pain:1.51), (10-HYDROXYCAMPTOTHECIN:Pain:0.11), (4-HYDROXYBUTANOIC ACID:Pain:0.43), (ABACAVIRLAMIVUDINEZIDOVUDINE:Pain:0.13), (ABALOPARATID E:Pain:0.97), (ACEPROMAZINE:Pain:0.59), (ACEPROMAZINE MALEATEACEPROMETAZINECLORAZEPATE DIPOTASSIUM:Pain:0.11), (ACETAMINOPHEN OR ACETAMINOPHEN\ASPIRIN\CAFFEINE OR ASPIRIN\CAFFEINE:Pain:0.67), (ACETAMINOPHENAPRONALIDECAFFEINEPROPYP HENAZONE:Pain:0.79), (ACETAMINOPHENASCORBIC ACIDPHENIRAMINE MALEATE:Pain:0.78), (ACETAMINOPHENBUCLIZINE HYDROCHLORIDECODEINE:Pain:0.93), (ACETAMINOPHENBUTABARBITALCAFFEI NE:Pain:0.74), (ACETAMINOPHENBUTYLSCOPOLAMINE BROMIDE:Pain:1.38), (ACETAMINOPHEN\CAFFEINEPHENYLEPHRINE HYDROCHLORIDE:Pain:0.34), (ACETAMINOPHENCHLORPHENIRAMINE MALEATEDEXTROMETHORPHAN HYDROBROMIDE:Pain:0.6), (ACETAMINOPHENCHLORPHENIRAMINE MALEATEDEXTROMETHORPHAN HYDROBROMIDEDOXYLAMINE SUCCINATEPHENYLEPHRINE HYDROCHLORIDE:Pain:0.3), (ACETAMINOPHENCHLORPHENIRAMINE MALEATEDEXTROMETHORPHAN HYDROBROMIDEPHENYLEPHRINE HYDROCHLORIDE OR ACETAMINOPHENDEXTROMETHORPHAN HYDROBROMIDEDOXYLAMINE SUCCINATEPHENYLEPHRINE HYDROCHLORIDE OR HOMEOPATHICS:Pain:1.01), (ACETAMINOPHENCHLORPHENIRAMINE MALEATEPSEUDOEPHEDRINE HYDROCHLORIDE:Pain:1.48), (ACETAMINOPHENDEXTROMETHORPHANDOXYLAM INEPSEUDOEPHEDRINE:Pain:0.29), (ACETAMINOPHENDICHLORALPHENAZONEISO METHEPTENE MUCATE:Pain:0.58), (ACETAMINOPHENDIPHENHYDRAMINE CITRATE:Pain:0.42), (ACETAMINOPHENPSEUDOEPHEDRINE HYDROCHLORIDE:Pain:0.6), (ACETIC ACIDCALCIUM CHLORIDEDEXTROSEMAGNESIUM CHLORIDE\POTASSIUM CHLORIDE\SODIUM CHLORIDE:Pain:0.57), (ACETIC ACIDSODIUM ACETATE:Pain:0.93), (ACETOHYDROXAMIC ACID:Pain:1.13), (ACETONE:Pain:0.5), (ACETYLCYSTEINEBENZALKONIUM CHLORIDETUAMINOHEPTANE SULFATE:Pain:1.14), (ACETYLDIGITOXIN:Pain:0.63), (ACIPIMOX:Pain:0.49), (ACLARUBI CIN:Pain:0.26), (ACLARUBICIN HYDROCHLORIDE:Pain:1.18), (ACYCLOVIRHYDROCORTISONE:Pain:0.34), (ADENINE: Pain:0.86), (ADENOSINE TRIPHOSPHATE:Pain:0.81), (ADENOSINE TRIPHOSPHATE DISODIUM:Pain:0.66), (ADUCANUMAB:Pain:0.78), (ALACEPRIL:Pain:0.33), (ALANINEAR GININEASPARTIC ACIDCYSTEINE HYDROCHLORIDEGLUTAMIC ACIDGLYCINEHISTIDINEISOLEUCINELEUCINELYSINE ACETATEMETHIONINEN-ACETYL-TYROSINEPHENYLALANINEPROLINESERINETAURINETHREONINETRYPTOPHA NTYROSINEVALINE:Pain:0.13), (ALANINEARGININEGLYCINEHISTIDINEISOLEUC INELEUCINELYSINE HYDROCHLORIDEMETHIONINEPHENYLALANINEPROLINESERINETHREONINE TRYPTOPHANTYROSINEVALINE:Pain:0.34), (ALBUTEROLGUAIFENESIN:Pain:1.11), (ALFALFAALFALFA WHOLE:Pain:0.67), (ALGINIC ACIDALUMINUM HYDROXIDEMAGNESIUM TRISILICATE:Pain:0.41), (ALISERTIB:Pain:0.7), (ALISKIREN HEMIFUMARATEVALSARTAN:Pain:1.33), (ALISKIRENHYDROCHLOROTHIAZIDE:Pai n:1.1), (ALISPORIVIR:Pain:0.59), (ALITRETINOIN:Pain:0.08), (ALLANTOIN:Pain:0.43), (ALL ANTOINOCTENIDINE:Pain:0.22), (ALOSETRON:Pain:1.03), (ALPHA-TOCOPHEROLASCORBIC ACIDCHOLECALCIFEROLCOPPERCYANOCOBALAMINFOLIC ACIDLUTEINOMEGA-3 FATTY ACIDSPYRIDOXINE HYDROCHLORIDETAURINEVITAMIN A PALMITATEZEAXANTHINZINC PICOLINATE:Pain:0.67), (ALPHA-TOCOPHEROLCHOLECALCIFEROL:Pain:0.87), (ALPROSTADILPAPAVERINEPHENTO LAMINE:Pain:0.69), (ALUMINUM PHOSPHATE:Pain:0.3), (ALUMINUM SILICATE:Pain:0.77), (ALVOCIDIB:Pain:0.47), (AMBENONIUM CHLORIDE:Pain:0.61), (AMILORIDE HYDROCHLORIDEHYDROCHLOROTHIAZIDETIMOLOL MALEATE:Pain:0.77), (AMINO ACIDSCARBOHYDRATESMINERALSSOYBEAN OILVITAMINS:Pain:1.4), (AMINO ACIDSVITAMINS:Pain:0.72), (AMINOBENZOIC ACIDCALCIUM PANTOTHENATECYSTINEKERATINTHIAMINE MONONITRATE:Pain:1.63), (AMINOLEVULINIC ACID:Pain:0.43), (AMINOPYRINEANTIPYRINECAFFEINEPHENACETIN:Pain:0.4), (AMI NOSALICYLATE SODIUM:Pain:0.69), (AMITRIPTYLINE HYDROCHLORIDECHLORDIAZEPOXIDE HYDROCHLORIDE:Pain:0.17), (AMLODIPINE BESYLATEPERINDOPRIL ARGININE:Pain:0.83), (AMLODIPINE MESYLATE:Pain:0.57), (AMLODIPINEATORVASTATIN:Pain:0.98), (AMLODIPINEINDA PAMIDE:Pain:0.39), (AMLODIPINELOSARTAN POTASSIUM:Pain:0.81), (AMMONIA:Pain:0.25), (AMOBARBITAL:Pain:0.44), (AMOROLFIN E:Pain:0.93), (AMOROLFINE HYDROCHLORIDE:Pain:0.35), (AMPHETAMINE ASPARTATE MONOHYDRATEAMPHETAMINE SULFATEDEXTROAMPHETAMINE SACCHARATEDEXTROAMPHETAMINE SULFATE:Pain:0.89), (AMPHETAMINE ASPARTATEDEXTROAMPHETAMINE SACCHARATE:Pain:0.98), (AMPHETAMINE SULFATE:Pain:1.13), (AMPICILLIN TRIHYDRATE:Pain:0.03), (AMRUBICIN:Pain:0.59), (AMRUBICIN HYDROCHLORIDE:Pain:0.7), (AMYLASE:Pain:0.75), (ANGIOTENSIN II:Pain:0.85), (ANHYDROUS CITRIC ACIDBICARBONATE IONMAGNESIUM CATION:Pain:0.16), (ANHYDROUS CITRIC ACIDMAGNESIUM OXIDE\SODIUM PICOSULFATE:Pain:1.16), (ANHYDROUS CITRIC ACIDPOTASSIUM BICARBONATE\SODIUM BICARBONATE:Pain:0.41), (ANHYDROUS DEXTROSECALCIUM CHLORIDELACTIC ACIDMAGNESIUM CHLORIDE\POTASSIUM CHLORIDE\SODIUM BICARBONATE\SODIUM CHLORIDE:Pain:0.1), (APATINIB:Pain:0.16), (APOMORPHINE:Pain:0.69), (APRACLONIDIN E HYDROCHLORIDE:Pain:0.26), (APRINDINE HYDROCHLORIDE:Pain:0.75), (APROTININ:Pain:0.3), (ARBEKACIN:Pain:0.44), (ARCTOST APHYLOS UVA-URSI LEAF DIOICA:Pain:1.24), (ARFORMOTEROL:Pain:1.28), (MONTANA:Pain:0.93), (ARNICA MONTANA FLOWER:Pain:0.55), (AROTINOLOL:Pain:0.98), (ARSENIC:Pain:0.21), (ARTESUNATE:Pain: 0.79), (ARTICAINE:Pain:0.05), (ASCORBIC ACID/CHROMIUM/SELENIUM/VANADYL SULFATE:Pain:1.72), (ASCORBIC ACIDCYANOCOBALAMINFOLIC ACIDIRON:Pain:0.99), (ASCORBIC ACIDFERROUS FUMARATEFOLIC ACID:Pain:0.91), (ASCORBIC ACIDHESPERIDIN METHYLCHALCONERUSCUS ACULEATUS ROOT:Pain:0.3), (ASCORBIC ACIDNIACINAMIDEPYRIDOXINE\RIBOFLAVINTHIAMINE:Pain:0.44), (ASCORBIC ACIDVITAMIN B COMPLEX:Pain:0.92), (ASPARTAME:Pain:0.96), (ASPARTIC ACID:Pain:0.99), (ASPIRIN SODIUM:Pain:0.63), (ASPIRIN\CAFFEINEPROMETHAZINESALICYLAMIDE:Pain:0.4), (A SPIRINCALCIUM CARBONATE:Pain:0.15), (ASPIRINCHLORPHENIRAMINE MALEATEPHENYLEPHRINE BITARTRATE:Pain:0.69), (ASPIRINDEXTROMETHORPHAN HYDROBROMIDEDOXYLAMINE SUCCINATEPHENYLEPHRINE BITARTRATE:Pain:0.51), (ASPIRINLANSOPRAZOLE:Pain:0.96), (ASPIRIN\SODIUM BICARBONATE:Pain:0.77), (ATAZANAVIRRITONAVIR:Pain:0.72), (ATROPINE SULFATEDIFENOXIN HYDROCHLORIDE:Pain:1.27), (AUTOLOGOUS CULTURED CHONDROCYTES:Pain:0.16), (AUTOLOGOUS CULTURED CHONDROCYTESSCROFA COLLAGEN:Pain:0.48), (AVOBENZONEECAMSULEOCTOCRYLENE:Pain:0.14), (AVOBE NZONEHOMOSALATEOCTISALATE:Pain:1.29), (AZASETRON:Pain:0.4), (AZASETRON HYDROCHLORIDE:Pain:0.86), (BACILLUS CALMETTE-GUERIN SUBSTRAIN CONNAUGHT LIVE ANTIGEN:Pain:0.47), (BACITRACINNEOMYCIN SULFATE:Pain:0.96), (BACITRACINNEOMYCIN SULFATEPOLYMYXIN B SULFATE:Pain:1.02), (BAMBUTEROL:Pain:0.33), (BARIUM:Pain:0.25), (BAZEDOXIFENE ACETATE:Pain:0.84), (BECLOMETHASONE DIPROPIONATEBETAMETHASONE SODIUM PHOSPHATE:Pain:0.75), (BENPERIDOL:Pain:0.45), (BENPROPERINE PHOSPHATE:Pain:0.59), (BENRALIZUMAB:Pain:0.79), (BENZOCAINEMENTHOL:Pain:0.45), (BENZPHETAMINE HYDROCHLORIDE:Pain:0.78), (BENZYL ALCOHOL:Pain:0.37), (BEPOTASTINEBEPOTASTINE BESILATE:Pain:1.01), (BEPRIDIL:Pain:0.95), (BEPRIDIL HYDROCHLORIDE:Pain:1.07), (BERACTANT:Pain:0.27), (BETAINE:Pain:1.04), (BETAMET HASONE DIPROPIONATECLOTRIMAZOLEGENTAMICIN SULFATE:Pain:0.83), (BEZLOTOXUMAB:Pain:0.48), (BICLOTYMOL:Pain:0.37), (BIOFLAV ONOIDS:Pain:0.79), (BIOTINCALCIUM PANTOTHENATECHOLINE BITARTRATECYANOCOBALAMINFOLIC ACIDINOSITOLNIACINAMIDEPYRIDOXINE\RIBOFLAVINTHIAMINE:Pain:0.88), (BIP ERIDEN LACTATE:Pain:0.61), (BISMUTH SUBCITRATE:Pain:0.31), (BISMUTH SUBSALICYLATEMETRONIDAZOLETETRACYCLINE HYDROCHLORIDE:Pain:0.85), (BIXALOMER:Pain:0.83), (BLOOD, BOVINE:Pain:0.61), (BOTULINUM TOXIN NOS:Pain:0.28), (BOTULISM ANTITOXIN:Pain:0.92), (BRIMONIDINE TARTRATEDORZOLAMIDE HYDROCHLORIDETIMOLOL MALEATE:Pain:0.68), (BRODALUMAB:Pain:0.77), (BROMISOVAL:Pain:0.14), (BROMPERI DOL:Pain:0.89), (BROMPHENIRAMINE MALEATEDEXTROMETHORPHAN HYDROBROMIDEPHENYLEPHRINE HYDROCHLORIDE:Pain:0.51), (BROMPHENIRAMINEPSEUDOEPHEDRINE:Pain:0.59), (B UCLIZINE HYDROCHLORIDE:Pain:0.32), (BUCOLOME:Pain:0.64), (BUDESONIDEINDACATEROL MALEATE:Pain:0.9), (BUFORMIN:Pain:0.96), (BUNAZOSIN HYDROCHLORIDE:Pain:0.06), (BUPIVACAINE HYDROCHLORIDEFENTANYL CITRATE:Pain:0.02), (BUPRANOLOL:Pain:0.3), (BUSERELIN ACETATE:Pain:0.3), (BUTABARBITAL:Pain:0.99), (BUTAMIRATE CITRATE:Pain:0.46), (BUTOCONAZOLE NITRATE:Pain:0.27), (CABOTEGRAVIR:Pain:0.57), (CAFFEINEINDOMETHACINPROCH LORPERAZINE MALEATE:Pain:0.24), (CAJUPUT OILEUCALYPTUS OILPEPPERMINT OIL:Pain:1.31), (CALCIUM ACETATEMAGNESIUM ACETATE\POTASSIUM ACETATE\SODIUM ACETATE\SODIUM CHLORIDE:Pain:1.24), (CALCIUM CARBIMIDE:Pain:1.15), (CALCIUM CARBONATEDIMETHICONE:Pain:0.79), (CALCIUM CARBONATEMAGNESIUM CARBONATE:Pain:0.71), (CALCIUM CHLORIDEDEXTROSE MONOHYDRATEHYDROCHLORIC ACIDMAGNESIUM CHLORIDE\SODIUM BICARBONATE\SODIUM CHLORIDE\SODIUM HYDROXIDE\SODIUM LACTATE:Pain:0.29), (CALCIUM CHLORIDEMAGNESIUM CHLORIDE\POTASSIUM CHLORIDE\SODIUM BICARBONATE\SODIUM CHLORIDE\SODIUM PHOSPHATE, DIBASIC:Pain:0.29), (CALCIUM CLDEXTROSEMAGNESIUM CLSODIUM BICARSODIUM CLSODIUM LACT:Pain:0.36), (CALCIUM CLMAGNESIUM CLPOTASSIUM CLSODIUM BICARSODIUM CLSODIUM PHOSPHATE,DIB:Pain:0.6), (CALCIUM DOBESILATE:Pain:0.65), (CALCIUM LACTATECALCIUM PHOSPHATECHOLECALCIFEROL:Pain:0.76), (CALCIUM MONOETHYLFUMARATEDIMETHYL FUMARATEMAGNESIUM MONOETHYLFUMARATE\ZINC MONOETHYLFUMARATE:Pain:0.29), (CALCIUM PANTOTHENATENIACINAMIDEPYRIDOXINE HYDROCHLORIDE\RIBOFLAVINTHIAMINE MONONITRATE:Pain:0.72), (CALCIUM PHOSPHATE:Pain:1.76), (CALCIUM PHOSPHATE, DIBASIC, ANHYDROUSFERROUS SUCCINATEMAGNESIUM HYDROXIDE:Pain:0.62), (CALCIUM SULFATEHERBALS:Pain:0.37), (CALCIUMCHOLECALCIFEROLMAGNESIUM\ZINC:Pa in:0.78), (CAMOSTAT:Pain:0.21), (CAMPHORCAPSICUMMETHYL SALICYLATE:Pain:0.75), (CAMPHORMENTHOLMETHYL SALICYLATE:Pain:0.89), (CAMPHORATED PHENOL:Pain:0.28), (CANDESARTAN CILEXETILFELODIPINE:Pain:0.75), (CANDIDA ALBICANS:Pain:0.12), (SATIVA SEED OIL:Pain:0.97), (CANRENOATE POTASSIUMTROMETHAMINE:Pain:0.04), (CAPECITABINEOXALIPLATIN:Pain:1.02), (C APREOMYCIN:Pain:0.67), (CAPSICUM:Pain:1.03), (CARBOMERHOMOPOLYMERTYPE C (ALLYL PENTAERYTHRITOL CROSSLINKED):Pain:0.6), (CARBON DIOXIDE:Pain:0.94), (CARBOPLATINPACLITAXEL:Pain:1.1), (CARGLUMIC ACID:Pain:0.58), (CARTEOLOL:Pain:0.52), (CARVEDILOL HYDROCHLORIDE:Pain:0.59), (CEFAMANDOLE:Pain:0.86), (CEFATRIZINE:Pain:0.61), (CE FCAPENE:Pain:0.32), (CEFCAPENE PIVOXIL:Pain:0.84), (CEFDITOREN:Pain:0.71), (CEFOPERAZONE SODIUM:Pain:0.53), (CEFOPERAZONE SODIUMSULBACTAM:Pain:0.55), (CEFOZOPRAN:Pain:0.06), (CEFPIROME:Pain:0.39), (CE FPIROME SULFATE:Pain:1.03), (CEFTERAM PIVOXIL:Pain:0.63), (CEFTIBUTEN DIHYDRATE:Pain:0.45), (CEFTIZOXIME:Pain:0.47), (CELECOXIBIBUPROFENNAPROXE NPLACEBO:Pain:0.56), (CELERY SEED:Pain:0.65), (CELLULOSE, OXIDIZED:Pain:1.1), (CEP-9722:Pain:0.29), (CEPHRADINE:Pain:0.78), (CERIVASTATIN:Pain:0.69), (CERIVASTATIN SODIUM:Pain:1.59), (CHENODIOL:Pain:0.44), (CHLOPHEDIANOL HYDROCHLORIDEDEXBROMPHENIRAMINE MALEATEPHENYLEPHRINE HYDROCHLORIDE:Pain:1.07), (CHLORAMPHENICOL PALMITATE:Pain:0.74), (CHLORHEXIDINE ACETATE:Pain:0.26), (CHLORINE:Pain:0.46), (CHLORODEHYDROMETHYLTESTOSTER ONE:Pain:0.05), (CHLOROXYLENOL:Pain:0.26), (CHLORPHENIRAMINE HYDROCHLORIDEHYDROCODONE:Pain:0.31), (CHLORPHENIRAMINE MALEATEIBUPROFENPSEUDOEPHEDRINE HYDROCHLORIDE:Pain:0.59), (CHLORPROMAZINE HIBENZATE:Pain:0.43), (CHLORPROPAMIDE:Pain:0.45), (CHOLIC ACID:Pain:0.85), (CHOLINE:Pain:0.93), (CHOLINE BITARTRATECYSTEINEINOSITOLMAMMAL LIVER:Pain:0.65), (CHOLINETRAZODONE HYDROCHLORIDE:Pain:1.2), (CHROMIC CATIONCUPRIC CATIONFERROUS CATIONFLUORIDE IONIODIDE IONMANGANESE CATION (2+)SODIUM SELENIDE\ZINC CATION:Pain:0.66), (CHROMIC CHLORIDECUPRIC CHLORIDEMANGANESE CHLORIDE\ZINC CHLORIDE:Pain:0.2), (CILASTATIN SODIUM:Pain:0.63), (CILAZAPRIL ANHYDROUSHYDROCHLOROTHIAZIDE:Pain:0.91), (CILENGITIDE:Pain:0.17), (CINNA MON OIL:Pain:0.99), (CIPROFLOXACINDEXAMETHASONE:Pain:0.81), (CITICOLINE SODIUM:Pain:0.97), (CITRIC ACID MONOHYDRATEDEXTROSE\SODIUM CITRATE\SODIUM PHOSPHATE, MONOBASIC:Pain:0.08), (CITRIC ACID MONOHYDRATE\POTASSIUM CITRATE:Pain:0.7), (CITRIC ACID MONOHYDRATETRISODIUM CITRATE DIHYDRATE:Pain:1.15), (CIXUTUMUMAB:Pain:0.64), (CLARITHROMYCIN LACTOBIONATE:Pain:0.7), (CLAVULANATE POTASSIUMTICARCILLIN DISODIUM:Pain:0.91), (CLENBUTEROL:Pain:0.13), (CLENBUTEROL HYDROCHLORIDE:Pain:0.94), (CLEVIDIPINE:Pain:0.62), (CLIDINIUM BROMIDE:Pain:1.01), (CLINDAMYCIN PALMITATE HYDROCHLORIDE:Pain:0.64), (CLINDAMYCIN PHOSPHATETRETINOIN:Pain:0.32), (CLIOQUINOL:Pain:0.34), (CLOBETASONE BUTYRATENYSTATINOXYTETRACYCLINE CALCIUM:Pain:0.7), (CLOCORTOLONE PIVALATE:Pain:1.33), (CLOPENTHIXOL HYDROCHLORIDE:Pain:0.23), (CLOPERASTINE:Pain:0.81), (CLOPIDOGREL HYDROBROMIDE:Pain:0.58), (CLOPIDOGREL HYDROCHLORIDE:Pain:0.74), (CLOPREDNOL:Pain:1.04), (CLORAZEPIC ACID:Pain:0.57), (CLOSTRIDIUM TETANICLOSTRIDIUM TETANI TOXOID ANTIGEN (FORMALDEHYDE INACTIVATED)DIPHTHERIAECORYNEBACTERIUM DIPHTHERIAE TOXOID ANTIGEN (FORMALDEHYDE INACTIVATED):Pain:1.45), (COAGULATION FACTOR IX RECOMBINANT HUMAN:Pain:0.41), (COAGULATION FACTOR VII HUMAN:Pain:0.52), (COAGULATION FACTOR VIIA (RECOMBINANT):Pain:0.49), (COCAINE HYDROCHLORIDE:Pain:0.12), (CODEINE HYDROCHLORIDE:Pain:0.77), (COLD CREAM:Pain:0.56), (COLESTILAN CHLORIDE:Pain:0.91), (COLISTIN A SODIUM METHANESULFONATE:Pain:0.75), (COPPERINTRAUTERINE DEVICE:Pain:0.64), (COUMARIN:Pain:1.22), (CROFELEMER:Pain:0.97), (CUPRIC SULFATE ANHYDROUS:Pain:0.42), (CURCUMINDIETARY SUPPLEMENT:Pain:0.11), (CYANAMIDE:Pain:0.58), (CYANOCOBALAMINPYRIDOXINE HYDROCHLORIDE\RIBOFLAVIN\SODIUM PANTOTHENATETHIAMINE HYDROCHLORIDEVITAMIN B COMPLEX:Pain:1.35), (CYCLOPENTOLATE HYDROCHLORIDEPHENYLEPHRINE HYDROCHLORIDE:Pain:0.07), (CYCLOPHOSPHAMIDEDOXORUBICINPREDNISONEV INCRISTINE:Pain:0.31), (CYCLOPHOSPHAMIDEEPIRUBICIN HYDROCHLORIDEFLUOROURACIL:Pain:0.48), (CYSTEAMINE:Pain:0.63), (CYSTEAMIN E HYDROCHLORIDE:Pain:0.16), (CYTARABINE HYDROCHLORIDE:Pain:0.56), (CYTARABINE OCFOSFATE:Pain:0.75), (CYTARABINEDAUNORUBICIN:Pain:0.76), (DABRAFENIBTRA METINIB:Pain:0.19), (DACETUZUMAB:Pain:0.28), (DALBAVANCIN HYDROCHLORIDE:Pain:0.08), (DALFOPRISTINQUINUPRISTIN:Pain:0.56), (DASABUVIR SODIUM MONOHYDRATE:Pain:0.62), (DASABUVIROMBITASVIRPARITAPREVIRRIBAVIRIN RITONAVIR:Pain:0.6), (DAUNORUBICIN CITRATE:Pain:0.55), (DENILEUKIN DIFTITOX:Pain:0.75), (DENOPAMINE:Pain:0.85), (DEOXYRIBONUCLIEC ACID:Pain:0.89), (DEPRODONE PROPIONATE:Pain:0.93), (DEUTETRABENAZINE:Pain:1.03), (DEXAMETHASONENEOM YCINPOLYMYXIN B SULFATE:Pain:0.46), (DEXPANTHENOLNIACINAMIDEPYRIDOXINE HYDROCHLORIDE\RIBOFLAVINTHIAMINE HYDROCHLORIDE:Pain:0.36), (DEXPANTHENOLXYLOMETAZOLINE HYDROCHLORIDE:Pain:0.22), (DEXTRAN 70GLYCERINHYPROMELLOSES:Pain:0.12), (DEXTRANHYPROMELLOSES:Pain:0.42), (DEXTROMETHORPHAN HYDROBROMIDEDOXYLAMINE SUCCINATE:Pain:0.31), (DEXTROSEFRUCTOSETRISODIUM CITRATE DIHYDRATE:Pain:0.72), (DEXTROSEHEPARIN:Pain:0.35), (DEXTROSE\POTASSIUM CHLORIDE\SODIUM CHLORIDE\SODIUM LACTATE:Pain:1.01), (DIFLUCORTOLONE VALERATELIDOCAINE:Pain:0.54), (DIHYDROTACHYSTEROL:Pain:0.86), (DILAZEP DIHYDROCHLORIDE:Pain:0.55), (DILOXANIDE FUROATETINIDAZOLE:Pain:0.16), (DIMETHYL SULFONEGLUCOSAMINE SULFATE:Pain:0.9), (DIPHTHERIA AND TETANUS TOXOIDS AND POLIOVIRUS VACCINE ANTIGENS:Pain:0.22), (DISODIUM FUMARATE:Pain:0.64), (DOCETAXEL ANHYDROUS:Pain:0.77), (DOCONEXENTICOSAPENTPHOSPHATIDYL SERINE:Pain:0.58), (DONEPEZIL HYDROCHLORIDEMEMANTINE HYDROCHLORIDE:Pain:0.96), (DOXIFLURIDINE:Pain:0.67), (DROPERIDOLFENTANYL CITRATE:Pain:0.02), (DUVELISIB:Pain:0.6), (DYCLONINE HYDROCHLORIDE:Pain:0.96), (ECABET:Pain:1.01), (ECABET SODIUM:Pain:0.95), (EFALIZUMAB:Pain:0.92), (ELECTROLYTES NOSMINERALSSORBITOL:Pain:0.25), (EMACTUZUMAB:Pain:0.79), (ENASIDENIB MESYLATE:Pain:0.86), (ENBUCRILATE:Pain:0.67), (ENDOSTATIN:Pain:0.44), (ENTOSPLE TINIB:Pain:0.44), (EPALRESTAT:Pain:0.74), (EPHEDRA:Pain:00.88), (EPHEDRINE HYDROCHLORIDEGUAIFENESIN:Pain:1.12), (EPHEDRINE SULFATE:Pain:0.78), (EPROSARTAN:Pain:0.94), (EPROSARTAN MESYLATEHYDROCHLOROTHIAZIDE:Pain:0.94), (EQUISETUM ARVENSE TOP:Pain:0.09), (ERGOCALCIFEROLPHYTONADIONERETINOLTOCOPHEROL:Pain:0.39), (ERGONOVINE MALEATE:Pain:0.04), (ERGONOVINEOXYTOCIN:Pain:0.21), (ERGOTAMINE TARTRATE:Pain:0.52), (ERYTHROMYCIN ESTOLATE:Pain:0.5), (ESCIN:Pain:0.57), (ESTRADIOL BENZOATE:Pain:1.75), (ESTRADIOL HEMIHYDRATE:Pain:1.15), (ESTRADIOL HEMIHYDRATENORETHINDRONE ACETATE:Pain:0.96), (ESTRADIOL VALERATENORETHINDRONE:Pain:0.23), (ESTRAMUSTINE PHOSPHATE:Pain:0.54), (ETELCALCETIDE:Pain:0.43), (ETEPLIRSEN:Pain:0.93), (ETHACR YNIC ACID:Pain:2.08), (ETHAMBUTOL HYDROCHLORIDEISONIAZIDPYRAZINAMIDERIFAMPIN:Pain:0.51), (ETHAMBUTOL HYDROCHLORIDEISONIAZIDPYRAZINAMIDERIFAMYCIN:Pain:0.58), (ETHAMBUT OLISONIAZIDPYRAZINAMIDERIFAMYCIN:Pain:0.72), (ETHINYL ESTRADIOLETHYNODIOL:Pain:1.01), (ETHINYL ESTRADIOLFERROUS FUMARATENORETHINDRONE:Pain:1.34), (ETHYLENE GLYCOL:Pain:0.19), (ETHYLMORPHINE:Pain:0.09), (ETHYLMORPHINE HYDROCHLORIDEHERBALS:Pain:0.37), (ETHYLPHENIDATE:Pain:0.53), (ETIMICIN SULFATE:Pain:1.11), (ETRETINATE:Pain:1.47), (EUCALYPTUS OIL:Pain:0.82), (EUGENOL:Pain:0.04), (EVOFOSFAMIDE:Pain:0.99), (EZETIMIBEROSUVA STATIN:Pain:1.13), (FALDAPREVIR:Pain:0.72), (FARLETUZUMAB:Pain:0.57), (FECAL MICROBIOTA:Pain:1.12), (FENOFIBRATEPRAVASTATIN:Pain:0.51), (FENOPROFEN:Pain: 0.98), (FENRETINIDE:Pain:0.55), (FENSPIRIDE HYDROCHLORIDE:Pain:0.74), (FERRIC SODIUM CITRATE:Pain:0.66), (FERRIC SULFATE:Pain:0.42), (FERROUS FUMARATEMESTRANOLNORETHINDRONE:Pain:0.51), (FERROUS SULFATEGASTRIC MUCIN:Pain:0.46), (FERUMOXYTOL:Pain:0.59), (FILANESIB:Pain:0.85), (FLIBANSERIN:Pa in:0.9), (FLOPROPIONE:Pain:0.85), (FLORBETAPIR F-18:Pain:0.87), (FLOXURIDINE:Pain:0.62), (FLUMETHASONE PIVALATE:Pain:0.61), (FLUOCORTOLONE:Pain:0.41), (FLUORESCEIN SODIUM:Pain:0.15), (FLUOROMETHOLONE ACETATE:Pain:0.18), (FLUOXYMESTERONE:Pain:1.03), (FLUPENTIXOL DIHYDROCHLORIDE:Pain:0.72), (FLUPHENAZINE DECANOATE:Pain:0.74), (FLUPREDNIDENE ACETATEMICONAZOLE NITRATE:Pain:1.11), (FLURAZEPAM MONOHYDROCHLORIDE:Pain:0.1), (FLUTEMETAMOL F-18:Pain:0.23), (FLUTICASONE FUROATEUMECLIDINIUM BROMIDEVILANTEROL TRIFENATATE:Pain:0.17), (FLUTOPRAZEPAM:Pain:0.38), (FORMALDEHYDE:Pain:1.03), (FOSBRETABULIN TROMETHAMINE:Pain:0.96), (FRANGULA PURSHIANA BARK:Pain:1.1), (FUCUS VESICULOSUS:Pain:0.58), (FUMARIC ACID:Pain:0.35), (FURAZOLIDONE:Pain:0.56), (FUROSEMIDE SODIUMSPIRONOLACTONE:Pain:0.6), (FURSULTIAMINE:Pain:1.06), (GADOLINIUM:Pai n:0.86), (GADOTERIDOL:Pain:0.09), (GANITUMAB:Pain:0.38), (GIMERACIL:Pain:0.34), (GI MERACILOTERACIL POTASSIUMTEGAFUR:Pain:0.76), (GINKGO BILOBA LEAF EXTRACT:Pain:0.89), (GINSENG EXTRACT:Pain:0.83), (GLUCONATE:Pain:1.03), (GLUCUROLACTONE:Pain:0.5), (GLYCER INMEDIUM-CHAIN TRIGLYCERIDESPHOSPHOLIPIDSOYBEAN OIL:Pain:0.5), (GLYCERINMINERAL OILPARAFFIN:Pain:0.87), (GLYCERINPROPYLENE GLYCOL:Pain:0.2), (GLYCEROL PHENYLBUTYRATE:Pain:0.22), (GLYCINEMANNITOLSODIUM CHLORIDE:Pain:0.04), (GOTU KOLA:Pain:0.98), (GRAPEFRUIT:Pain:0.74), (GRAPEFRUIT JUICE:Pain:0.65), (GRAPEFRUIT SEED EXTRACT:Pain:0.94), (GUAIAZULENE:Pain:0.65), (GUANABENZ ACETATE:Pain:0.65), (GUSPERIMUS TRIHYDROCHLORIDE:Pain:1.23), (INFLUENZAE TYPE B:Pain:0.42), (HALOTHANE:Pain:0.62), (HAWTHORN LEAF WITH FLOWER:Pain:0.9), (HEPARIN SODIUMSODIUM CHLORIDE:Pain:0.32), (HEPATITIS A VIRUS STRAIN CR 326F ANTIGEN (FORMALDEHYDE INACTIVATED):Pain:0.05), (HEPATITIS A VIRUS STRAIN HM175 ANTIGEN (FORMALDEHYDE INACTIVATED)HEPATITIS B VIRUS SUBTYPE ADW2 HBSAG SURFACE PROTEIN ANTIGEN:Pain:0.74), (HEPTAMINOL:Pain:0.57), (HERBALSHOMEOPATHICSGOLDENS EAL:Pain:1.23), (HERBALSKARAYA GUM:Pain:1.21), (HERBALSVITIS VINIFERA SEED:Pain:0.82), (HERBSROOTS:Pain:0.49), (HERBSUNSPECIFIED INGREDIENT:Pain:0.86), (HESPERIDIN:Pain:1.28), (HEXAMIDINE DIISETHIONATE:Pain:0.11), (HOMEOPATHICSZINC ACETATE ANHYDROUSZINC GLUCONATE:Pain:0.1), (HOMEOPATHICSZINC ACETATE\ZINC GLUCONATE:Pain:0.35), (HUMAN CLOSTRIDIUM TETANI TOXOID IMMUNE GLOBULIN:Pain:0.55), (HUMAN COLOSTRUM:Pain:1.12), (HUMAN VARICELLA-ZOSTER IMMUNE GLOBULIN:Pain:0.52), (HYALURONIC ACIDLIDOCAINE HYDROCHLORIDE:Pain:0.03), (HYALURONIDASE (HUMAN RECOMBINANT)RITUXIMAB:Pain:0.2), (HYDROCHLORIC ACID:Pain:1.26), (HYDROCHLOROTHIAZIDEZOFENOPRIL:Pain:0.62), (HYDROCHLORO THIAZIDEZOFENOPRIL CALCIUM:Pain:0.76), (HYDROCORTISONE HEMISUCCINATE:Pain:1.36), (HYDROCORTISONE PROBUTATE:Pain:0.84), (HYDROCORTISONEPRAMOXINE:Pain:0.79), (HYDROXYPROG ESTERONE:Pain:0.2), (HYPOCHLORITE ION:Pain:0.12), (IBUDILAST:Pain:0.81), (IDEBENONE:Pain:0.65), (ILOPROST TROMETHAMINE:Pain:0.15), (IMIPRAMINE PAMOATE:Pain:1.19), (INDAPAMIDE HEMIHYDRATE:Pain:0.41), (INDIGOTINDISULFONATE SODIUM:Pain:0.41), (INDISETRON HYDROCHLORIDE:Pain:0.65), (INDIUM IN-111 CHLORIDEINDIUM IN-111 PENTETREOTIDEPENTETREOTIDE:Pain:0.36), (INDOBUFEN:Pain:0.71), (INDORAMIN:P ain:0.46), (INFLUENZA A VIRUS A/BRISBANE/10/2010 (H1N1) ANTIGEN (MDCK CELL DERIVED, PROPIOLACTONE INACTIVATED)\INFLUENZA A VIRUS A/VICTORIA/361/2011 IVR-165 (H3N2) ANTIGEN (MDCK CELL DERIVED, PROPIOLACTONE INACTIVATED)INFLUENZA B VIRUS B/WISCONSIN/1/2010) ANTIGEN (MDCK CELL DERIVED, PROPIOLACTONE INACTIVATED):Pain:0.89), (INFLUENZA A VIRUS A/CALIFORNIA/7/2009 (H1N1) RECOMBINANT HEMAGGLUTININ ANTIGEN\INFLUENZA A VIRUS A/VICTORIA/361/2011 (H3N2) RECOMBINANT HEMAGGLUTININ ANTIGENlNFLUENZA B VIRUS B/WISCONSIN/1/2010 RECOMBINANT HEMAGGLUTININ ANTIGEN:Pain:0.69), (INFLUENZA VIRUS VACCINE, SWINE:Pain:0.24), (INIPARIB:Pain:0.31), (INOSINE:Pain:0.7), (INOSINE PRANOBEX:Pain:0), (INSULIN ASPARTINSULIN DEGLUDEC:Pain:0.85), (INSULIN DEGLUDECLIRAGLUTIDE:Pain:0.83), (INSULIN GLARGINELIXISENATIDE:Pain:0.62), (INTERFERON ALFACON-1:Pain:0.85), (INTERFERON GAMMA:Pain:0.92), (IOBENGUANE I-123:Pain:0.12), (IOBITRIDOL:Pain:0.96), (IODINETOSITUMOMAB:Pain:0.58), (IOPROMIDE: Pain:0.16), (IOTHALAMIC ACID:Pain:0.5), (IOXAGLATE MEGLUMINEIOXAGLATE SODIUM:Pain:0.07), (IPRAGLIFLOZIN:Pain:0.71), (IPRAGLIFLOZIN L-PROLINE:Pain:1.01), (IRBESARTANTRICHLORMETHIAZIDE:Pain:0.43), (IRON ISOMALTOSIDE 1000:Pain:0.25), (IRONMINERALSVITAMINS:Pain:0.08), (ISEPAMICIN:Pain:0.72), (ISOCA RBOXAZID:Pain:0.91), (ISOFLAVONES SOY:Pain:1.16), (ISOLEUCINE:Pain:0.67), (ISONIAZIDPYRIDOXINE:Pain:0.63), (ISOPROP YL ALCOHOLTENECTEPLASE:Pain:0.51), (ISOPROPYL MYRISTATE:Pain:0.61), (ISOPROTERENOL:Pain:0.59), (ISTRADEFYLLINE:Pain:0.36), (JOS AMYCIN:Pain:0.39), (JWH-018:Pain:0.91), (KALLIDINOGENASE:Pain:0.73), (KERATIN:Pain:1.02), (KERATINSENNA LEAF:Pain:0.59), (KETANSERIN:Pain:0.32), (KONJAC MANNAN:Pain:0.96), (LACTIC ACID:Pain:1.1), (LACTOBACILLUS ACIDOPHILUSSTREPTOCOCCUS FAECAIS:Pain:0.72), (LAMIVUDINENEVIRAPINESTAVUDINE:Pain:0.2), (LANINAMIVI R:Pain:0.66), (LANOLINMINERAL OIL:Pain:0.65), (LATANOPROSTTIMOLOL:Pain:1.41), (LENTINAN:Pain:0.43), (LESINURA D:Pain:1.06), (LESTAURTINIB:Pain:0.79), (LETROZOLERIBOCICLIB:Pain:1.19), (LEUCINE: Pain:0.85), (LEUPROLIDE ACETATENORETHINDRONE ACETATE:Pain:0.6), (LEVAMISOLE:Pain:0.12), (LEVOCABASTINE HYDROCHLORIDE:Pain:0.96), (LEVOCARNITINE HYDROCHLORIDE:Pain:0.8), (LEVODOPAUNSPECIFIED INGREDIENT:Pain:0.41), (LEVOLEUCOVORIN:Pain:0.73), (LEVOLEUCOVORIN DISODIUM:Pain:0.27), (LEVOMEFOLATE CALCIUM\METHYLCOBALAMINPYRIDOXAL 5-PHOSPHATE:Pain:0.95), (LEVOMEPROMAZINE HYDROCHLORIDELEVOMEPROMAZINE MALEATE:Pain:0.6), (LINCOMYCIN:Pain:0.98), (LIRILUMAB:Pain:0.56), (LITHIUM CITRATE:Pain:0.53), (LITHIUM OROTATE:Pain:1.04), (LITHIUM SULFATE:Pain:0.74), (LUTETIUM LU-177:Pain:1.56), (LYSINE HYDROCHLORIDE:Pain:1.08), (MAGNESIUM ACETATE TETRAHYDRATE\POTASSIUM ACETATE\SODIUM CHLORIDE:Pain:0.32), (MAGNESIUM ALUMINUM SILICATE:Pain:0.88), (MAGNESIUM CHLORIDE\POTASSIUM CHLORIDE\POTASSIUM PHOSPHATE\SODIUM ACETATE\SODIUM CHLORIDE\SODIUM GLUCONATE\SODIUM PHOSPHATE, DIBASIC:Pain:0.28), (MAGNESIUM GLUCEPTATE:Pain:1.19), (MAGNESIUM HYDROXIDEPARAFFIN:Pain:0.59), (MAGNESIUM STEARATE:Pain:0.77), (MAGNESIUM SULFATE HEPTAHYDRATE:Pain:0.94), (MAGNESIUM TRISILICATE:Pain:0.31), (MALATHION:Pain:0.4), (MALTODEXTRIN:Pain:0.56), (MANGA NESE GLUCONATE:Pain:0.19), (MANIDIPINE HYDROCHLORIDE:Pain:1.38), (MARIZOMIB:Pain:1.11), (RECUTITA:Pain:1.18), (MAZINDOL:Pain:0.56), (MECHLORETHAMINE:Pain:0.09), (MECHL ORETHAMINE HYDROCHLORIDE:Pain:0.53), (MECHLORETHAMINE OXIDE HYDROCHLORIDE:Pain:0.8), (MECLIZINEPYRIDOXINE:Pain:0.13), (MEDIUM-CHAIN TRIGLYCERIDES:Pain:0.14), (MEGLUMINE:Pain:0.11), (MELDONIUM:Pain:1), (MELITRA CEN:Pain:0.35), (MENADIONE:Pain:0.65), (MEPENZOLATE BROMIDE:Pain:1.11), (MEPHENESIN:Pain:0.19), (MEROPENEM ANHYDROUS:Pain:0.57), (METAPROTERENOL:Pain:0.46), (METFORMINSITAGLIPTIN PHOSPHATE:Pain:0.32), (METFORMINVILDAGLIPTIN:Pain:0.85), (METHACHOLINE CHLORIDE:Pain:0.55), (METHENOLONE ENANTHATE:Pain:1.78), (METHOHEXITAL:Pain:0.49), (METHOTREXATEMETHOTREX ATE SODIUM:Pain:0.48), (METHOXYPHENAMINE:Pain:0.43), (METHSCOPOLAMINEMETHS COPOLAMINE BROMIDE:Pain:0.54), (METHSUXIMIDE:Pain:0.92), (METHYL ALCOHOL:Pain:0.75), (METHYLPREDNISOLONE OR METHYLPREDNISOLONE ACETATE:Pain:1.85), (METHYSERGIDE MALEATE:Pain:0.9), (METYROSINE:Pain:0.79), (MEZLOCILLIN SODIUM:Pain:0.06), (MIFAMURTIDE:Pain:1.16), (MILRINONE LACTATE:Pain:1.2), (MILTEFOSINE:Pain:0.05), (MINERAL OIL EMULSION:Pain:0.79), (MINERALSOMEGA-3 FATTY ACIDSVITAMINS:Pain:0.55), (MINERALSPROBIOTICS NOSVITAMINS:Pain:1.01), (MIRABEGRONSOLIFENACIN:Pain:0.53), (MIRIPLATIN:Pain: 0.38), (MITIGLINIDE CALCIUM HYDRATE:Pain:0.27), (MITIGLINIDE CALCIUM HYDRATEVOGLIBOSE:Pain:0.94), (MITOGUAZONE:Pain:0.39), (MITRAGYNINE:Pain:0.24), (MIVACURIUM CHLORIDE:Pain:0.41), (MIZOLASTINE:Pain:0.66), (MOLYBDENUM!:Pain:0.78), (MONOSO DIUM GLUTAMATE:Pain:0.6), (MOSAPRIDE CITRATE DIHYDRATE:Pain:1.05), (MYCOPHENOLATE MOFETIL HYDROCHLORIDE:Pain:0.73), (NADH:Pain:1.04), (NAFAMOSTAT:Pain:0.83), (NAFCILLIN SODIUM:Pain:0.16), (NALDEMEDINE:Pain:0.74), (NALFURAFINE HYDROCHLORIDE:Pain:0.77), (NALIDIXIC ACID:Pain:0.44), (NALMEFENE:Pain:0.54), (NANDROLONE:Pain:0.47), (NANDROLONE DECANOATE:Pain:0.11), (NESIRITIDE:Pain:0.58), (NETILMICIN:Pain:0.55), (NIACINAMID EPYRIDOXINE\RIBOFLAVINTHIAMINE:Pain:1.21), (NICERGOLINE:Pain:1.09), (NIMET AZEPAM:Pain:0.58), (NIMOTUZUMAB:Pain:0.32), (NIMUSTINE:Pain:1.01), (NIPRADILOL: Pain:0.51), (NITAZOXANIDE:Pain:1.22), (NOMEGESTROL:Pain:0.9), (NORETHANDROLON E:Pain:0.85), (NORGESTIMATE:Pain:0.74), (NORGESTREL:Pain:0.44), (OCTENIDINEPHEN OXYETHANOL:Pain:0.16), (OCTOTIAMINE:Pain:0.95), (OLODATEROLTIOTROPIUM BROMIDE:Pain:0.62), (OLSALAZINE:Pain:0.9), (OLSALAZINE SODIUM:Pain:0.5), (OMACETAXINE:Pain:0.93), (OMARIGLIPTIN:Pain:0.58), (OMBITASVI R:Pain:0.43), (OMBITASVIR HEMINONAHYDRATEPARITAPREVIR DIHYDRATERITONAVIR:Pain:0.96), (OPICAPONE:Pain:0.75), (OPRELVEKIN:Pain:0.92), (OREGANO:Pain:0.77), (ORNITHINE ASPARTATE:Pain:0.5), (OSTREA EDULIS SHELL:Pain:1), (OTERACIL:Pain:0.21), (OTERACIL POTASSIUM:Pain:0.4), (OVINE DIGOXIN IMMUNE FAB:Pain:0.41), (OXANDROLONE:Pain:1.26), (OXITROPIUM BROMIDE:Pain:0.35), (OXPRENOLOL:Pain:0.69), (OXYMETAZOLINE HYDROCHLORIDETETRACAINE HYDROCHLORIDE:Pain:0.44), (OXYMETHOLONE:Pain:0.62), (OXYPERTINE:Pain:0.83), (OXYQUINOLINE SULFATE\SODIUM LAURYL SULFATE:Pain:1.12), (OZAGREL:Pain:0.2), (PANCRELIPASETYROSINE:Pain:0.73), (PANI PENEM:Pain:0.55), (PARALDEHYDE:Pain:0.52), (PARITAPREVIR:Pain:0.38), (PARNAPARIN SODIUM:Pain:0.42), (PAROMOMYCIN:Pain:0.33), (PAROMOMYCIN SULFATE:Pain:0.49), (PARSLEY:Pain:1.34), (PASSIFLORA INCARNATA FLOWER:Pain:1.03), (PAZUFLOXACIN:Pain:0.91), (PAZUFLOXACIN MESILATE:Pain:1.32), (PEGINTERFERON ALFA-2BRIBAVIRIN:Pain:0.76), (PEGINTERFERON LAMBDA-A:Pain:0.5), (PEMOLINE:Pain:0.88), (PENICILLIN G PROCAINE:Pain:0.33), (PENTACHLOROPHENOL:Pain:0.14), (PENTETRAZOL:Pain:0.9), (P ENTOXYVERINE CITRATE:Pain:0.57), (PERAZINE:Pain:0.78), (PEROSPIRONE:Pain:0.7), (PEROSPIRONE HYDROCHLORIDE:Pain:0.81), (PHENCYCLIDINE:Pain:0.41), (PHENETHICILLIN POTASSIUM:Pain:0.7), (PHENOXYBENZAMINE:Pain:0.6), (PHENOXYMETHYLPENICILL IN CALCIUM:Pain:0.26), (PHENTOLAMINE:Pain:1.14), (PHENYLALANINE, DL-:Pain:0.69), (PHENYLBUTAZONE:Pain:0.67), (PHENYLEPHRINE HYDROCHLORIDEPREDNISOLONE ACETATE:Pain:0.26), (PHENYLPROPANOLAMINE:Pain:0.8), (PHOSPHATIDYL SERINE:Pain:0.92), (PICOLAMINE SALICYLATE:Pain:0.63), (PILSICAINIDE:Pain:1), (PIMAVANSERIN:Pain:0.65), (PIMOBEN DAN:Pain:1.48), (PIPER METHYSTICUM ROOT:Pain:0.58), (PIPERAZINE:Pain:0.3), (PIPOTIAZINE:Pain:0.6), (PIPOTIAZINE PALMITATE:Pain:0.54), (PIRARUBICIN HYDROCHLORIDE:Pain:0.84), (PIRENZEPINE HYDROCHLORIDE:Pain:0.7), (PIRMENOL HYDROCHLORIDE:Pain:0.68), (PIXANTRONE:Pain:0.4), (PLASMA PROTEIN FRACTION (HUMAN):Pain:1.36), (POLYSORBATE 80:Pain:0.57), (POTASSIUM BICARBONATE\POTASSIUM CITRATE:Pain:0.53), (POTASSIUM BITARTRATE:Pain:1.05), (POTASSIUM BROMIDE:Pain:0.92), (POTASSIUM CARBONATE\POTASSIUM CITRATE:Pain:0.62), (POTASSIUM CITRATE\SODIUM CITRATE:Pain:0.11), (POTASSIUM CITRATETRISODIUM CITRATE DIHYDRATE:Pain:1.36), (POTASSIUM HYDROXIDE\POTASSIUM PHOSPHATE, DIBASIC\SODIUM PHOSPHATE, DIBASIC, ANHYDROUS:Pain:0.58), (POTASSIUM SORBATE:Pain:0.7), (PRACINOSTAT:Pain:0.81), (PRAZIQUANTEL:Pain:0.11), (PREDNISO LONE HEMISUCCINATE:Pain:0.45), (PREDNISOLONE SODIUM METAZOATE:Pain:1.53), (PRILOCAINE HYDROCHLORIDE:Pain:0.91), (PROBUCOL:Pain:1.11), (PROCYCLIDINE HYDROCHLORIDE:Pain:0.96), (PROGUANIL:Pain:0.15), (PROMETHAZINE TEOCLATE:Pain:0.44), (PROPANTHELINE:Pain:1.06), (PROPOLIS WAX:Pain:0.86), (PROPYPHENAZONE:Pain:0.02), (PROTEASE:Pain:0.65), (PROTHIPENDY L:Pain:0.3), (PROTIONAMIDE:Pain:0.44), (PSORALEN:Pain:1.13), (PYRANTEL PAMOATE:Pain:0.46), (PYRIDOXAL PHOSPHATE:Pain:1.38), (PYRILAMINE:Pain:0.35), (QUERCUS SALICINA STEM:Pain:0.62), (QUINIDINE GLUCONATE:Pain:1.01), (QUINIDINE SULFATE:Pain:0.63), (RABIES VIRUS STRAIN FLURY LEP ANTIGEN (PROPIOLACTONE INACTIVATED):Pain:0.05), (RACEPINEPHRINE HYDROCHLORIDE:Pain:0.22), (RADIUM:Pain:0.99), (RALTITREXED:Pain:1.05), (REBOXE TINE MESYLATE:Pain:1.02), (RED CLOVER:Pain:1.42), (RETEPLASE:Pain:0.03), (RHAMNUS PURSHIANA BARK EXTRACT:Pain:0.33), (RHUBARB:Pain:1.32), (RIBONUCLEIC ACID:Pain:0.22), (RIFAPENTINE:Pain:0.39), (RIGOSERTIB:Pain:0.79), (RILMAZAFONE:Pai n:0.66), (RILONACEPT:Pain:1), (RILOTUMUMAB:Pain:1.03), (RILPIVIRINE:Pain:0.51), (RIM ANTADINE HYDROCHLORIDE:Pain:1.4), (RITODRINE HYDROCHLORIDE:Pain:0.09), (ROMOSOZUMAB:Pain:0.26), (ROSIGLITAZONE:Pain:0.72), (ROXATIDINE ACETATE HYDROCHLORIDE:Pain:0.79), (RUBIDIUM CHLORIDE RB-82:Pain:0.13), (RUPATADINE FUMARATE:Pain:1.68), (RUTIN:Pain:0.77), (SACROSIDASE:Pain:0.65), (SAFINAMIDE:Pain: 0.71), (SAGE:Pain:0.76), (SALICYLAMIDE:Pain:0.82), (SEBELIPASE ALFA:Pain:0.91), (SELINEXOR:Pain:0.62), (SERMORELIN ACETATE:Pain:0.65), (SERTACONAZOLE NITRATE:Pain:0.7), (SHEA BUTTER:Pain:0.13), (SILICON:Pain:0.89), (SILTUXIMAB:Pain:0.96), (SILVER NITRATE:Pain:0.76), (SIMEPREVIR SODIUM:Pain:0.56), (SIMVASTATINVALSARTAN:Pain:0.46), (SINCALIDE:Pain:0.22), (SIP ONIMOD:Pain:0.79), (SIRUKUMAB:Pain:0.85), (SOBUZOXANE:Pain:0.58), (SODIUM BENZOATE:Pain:0.43), (SODIUM BENZOATE\SODIUM PHENYLACETATE:Pain:0.42), (SODIUM FEREDETATE:Pain:0.57), (SODIUM FLUORIDETRICLOSAN:Pain:0.56), (SODIUM FLUORIDEVITAMINS:Pain:1.08), (SODIUM GLYCEROPHOSPHATE:Pain:0.32), (SODIUM HYDROXIDE:Pain:0.56), (SODIUM PHOSPHATE, DIBASIC, ANHYDROUS\SODIUM PHOSPHATE, MONOBASIC, MONOHYDRATE:Pain:1.09), (SODIUM SELENITE:Pain:0.64), (SOFALCONE:Pain:0.78), (SOMATREM:Pain:0.37), (SORAFENIB TOSYLATE:Pain:0.75), (SOY ISOFLAVONES:Pain:0.88), (STARCH, CORN:Pain:0.52), (STIRIPENTOL:Pain:0.36), (STREPTOKINASE:Pain:0.47), (STREPTOZOCI N:Pain:0.62), (SUCCIMER:Pain:0.52), (SUGAMMADEX SODIUM:Pain:0.87), (SULBACTAM:Pain:0.9), (SULBUTIAMINE:Pain:1.15), (SULODEXIDE: Pain:0.99), (SULTAMICILLIN TOSYLATE:Pain:0.47), (SULTHIAME:Pain:0.32), (SUPLATAST TOSILATE:Pain:1.22), (TABALUMAB:Pain:1.45), (TALTIRELIN:Pain:00.82), (TANDOSPIRONE CITRATE:Pain:0.76), (TANESPIMYCIN:Pain:0.48), (TANEZUMAB:Pain:0.84), (TAR:Pain:0.92), (TARAXACUM OFFICINALE POLLEN:Pain:0.86), (TEA TREE OIL:Pain:0.94), (TECELEUKIN:Pain:0.81), (TECHNETIUM TC-99M TETROFOSMIN:Pain:0), (TECHNETIUM TC-99M TILMANOCEPT:Pain:0.21), (TEDIZOLID:Pain:0.69), (TEGAFUR:Pain:0.75), (TEGAFURURA CIL:Pain:1.01), (TEMOCAPRIL:Pain:1.13), (TEMOCAPRIL HYDROCHLORIDE:Pain:0.99), (TEMOCILLIN:Pain:0.28), (TENECTEPLASE:Pain:0.44), (TE NIPOSIDE:Pain:0.8), (TENOFOVIR ALAFENAMIDE:Pain:0.01), (TENOFOVIR ALAFENAMIDE FUMARATE:Pain:0.47), (TENOFOVIR DISOPROXIL:Pain:0.33), (TETRACAINE:Pain:0.54), (TETRAHYDROZOLINE HYDROCHLORIDE\ZINC SULFATE:Pain:0.29), (THIAMINE MONONITRATE:Pain:0.32), (THIAMYLAL:Pain:0.89), (THIORIDAZINE HYDROCHLORIDE:Pain:0.69), (THROMBIN HUMAN:Pain:0.72), (THYMALFASIN:Pain:0.67), (THYROGLOBULIN:Pain:1.05), (TIAGABI NE:Pain:0.39), (TIANEPTINE SODIUM:Pain:0.89), (TIARAMIDE:Pain:0.75), (TICARCILLIN:Pain:0.76), (TIOPRONIN:Pain: 1.25), (TIROFIBAN HYDROCHLORIDE:Pain:0.03), (TIROPRAMIDE:Pain:0.85), (TOLMETIN:Pain:0.78), (TOLM ETIN SODIUM:Pain:1.25), (TOPIROXOSTAT:Pain:0.95), (TOREMIFENE:Pain:0.48), (TOSITUMOM ABTOSITUMOMAB I-131:Pain:0.39), (TOSUFLOXACIN TOSYLATE:Pain:1.39), (TRANYLCYPROMINE SULFATE:Pain:1.5), (TRAPIDIL:Pain:0.1), (TREBANANIB:Pain:0.52), (TRELAGLIPTIN SUCCINATE:Pain:0.19), (TRIBENOSIDE:Pain:0.66), (TRICLOSAN:Pain:0.93), (TRIENTINE:P ain:0.63), (TRIENTINE HYDROCHLORIDE:Pain:0.7), (TRIFLUSAL:Pain:0.75), (TRISODIUM CITRATE DIHYDRATE:Pain:0.01), (TROFOSFAMIDE:Pain:0.49), (TROPINE BENZYLATE:Pain:0.1), (TULOBUTEROL HYDROCHLORIDE:Pain:0.77), (TYPHOID VACCINE:Pain:0.68), (UBENIMEX:Pain:0.8), (ULIPRISTAL:Pain:0.74), (UNDECYLENIC ACID:Pain:0.89), (URACIL:Pain:1.24), (URELUMAB:Pain:0.81), (URIDINE TRIACETATE:Pain:0.55), (VALERIAN EXTRACT:Pain:0.69), (VALPROATE MAGNESIUM:Pain:0.51), (VANIPREVIR:Pain:0.3), (VELPATASVIR:Pain:0.12), (VERUPREV IR:Pain:0.37), (VINDESINE:Pain:0.62), (VITAMIN A PALMITATE:Pain:1.05), (VONOPRAZAN:Pain:1.06), (WINE:Pain:0.04), (XANTOFYL PALMITATE:Pain:0.65), (YOHIMBINE:Pain:0.56), (ZINC ACETATE ANHYDROUSZINC GLUCONATE:Pain:0.28), (ZINC ACETATE\ZINC GLUCONATE:Pain:0.28), (ZINC CITRATE:Pain:1.27), (ZOFENOPRIL:Pain:0.93), (ZOFENOPRIL CALCIUM:Pain:0.48), (ZOLEDRONATE DISODIUM:Pain:1.05), (ZOTEPINE:Pain:0.75), (ZUCLOPENTHIXOL HYDROCHLORIDE:Pain:0.54), (.ALPHA.1-PROTEINASE INHIBITOR HUMAN:Pain in extremity:0.78), (ABOBOTULINUMTOXINA:Pain in extremity:0.02), (ACEBUTOLOL HYDROCHLORIDE:Pain in extremity:0.22), (ACETAMINOPHEN\CAFFEINEOPIUM:Pain in extremity:0.16), (ACETAMINOPHENPROPOXYPHENE NAPSYLATE:Pain in extremity:0.84), (ACITRETIN:Pain in extremity:0.75), (ACTIVATED CHARCOAL:Pain in extremity:0.85), (ALBUMIN HUMAN:Pain in extremity:0.25), (ALDESLEUKIN:Pain in extremity:0.23), (ALFUZOSIN HYDROCHLORIDE:Pain in extremity:1.28), (AMINO ACIDSDEXTROSEELECTROLYTES NOS:Pain in extremity:0.96), (AMISULPRIDE:Pain in extremity:1.09), (AMLODIPINE BESYLATEIRBESARTAN:Pain in extremity:0.12), (AMMONIUM CHLORIDEDEXTROMETHORPHAN\SODIUM CITRATE:Pain in extremity:0.24), (AMOXICILLINCLARITHROMYCINESOMEPRAZOLE:Pain in extremity:0.23), (AMPICILLIN SODIUM:Pain in extremity:0.13), (ANAKINRA:Pain in extremity:0.04), (ANTIHISTAMINES NOS:Pain in extremity:0.72), (ARIPIPRAZOLE LAUROXIL:Pain in extremity:0.1), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Pain in extremity:0.06), (ASCORBIC ACIDBIOTINCYANOCOBALAMINFOLIC ACIDNIACINPANTOTHENIC ACIDPYRIDOXINE\RIBOFLAVINTHIAMINE:Pain in extremity:0.04), (ASPIRINMAGNESIUM OXIDE:Pain in extremity:0.49), (ATROPINEDIPHENOXYLATE:Pain in extremity:0.51), (AZELASTINE HYDROCHLORIDEFLUTICASONE PROPIONATE:Pain in extremity:0.13), (BECLOMETHASONE:Pain in extremity:0.45), (BENSERAZIDELEVODOPA:Pain in extremity:0.2), (BEPRIDIL:Pain in extremity:0.26), (BETAMETHASONE DIPROPIONATECALCIPOTRIENE:Pain in extremity:0.14), (BETAMETHASONE DIPROPIONATECALCIPOTRIENECALCIPOTRIENE HYDRATE:Pain in extremity:0.78), (BETAMETHASONECALCIPOTRIENE:Pain in extremity:0.69), (BILASTINE:Pain in extremity:0.04), (BLINATUMOMAB:Pain in extremity:0.78), (BOCEPREVIR:Pain in extremity:0.22), (CABOZANTINIB S-MALATE:Pain in extremity:0.32), (CALCIUM LACTATE:Pain in extremity:0.15), (CALCIUM\MAGNESIUM\ZINC:Pain in extremity:0.52), (CANAGLIFLOZINMETFORMIN HYDROCHLORIDE:Pain in extremity:1.44), (CARBIDOPA:Pain in extremity:0.56), (CARBOMER:Pain in extremity:0.06), (CARVEDILOL PHOSPHATE:Pain in extremity:2.68), (CASPOFUNGIN:Pain in extremity:0.32), (CEFOTAXIME SODIUM:Pain in extremity:1.15), (CERITINIB:Pain in extremity:0.65), (CILASTATIN SODIUMIMIPENEM:Pain in extremity:1.4), (CLEMASTINE FUMARATE:Pain in extremity:0.03), (CLOBAZAM:Pain in extremity:0.21), (CLOXAZOLAM:Pain in extremity:0.11), (COBICISTATELVITEGRAVIREMTRICITABINETENOFOVIR ALAFENAMIDE FUMARATE:Pain in extremity:0.13), (CORTICOTROPIN:Pain in extremity:1.05), (CURCUMIN:Pain in extremity:0.06), (CYANOCOBALAMINFOLIC ACIDPYRIDOXINE:Pain in extremity:0.15), (CYPROHEPTADINE HYDROCHLORIDE:Pain in extremity:0.05), (DABRAFENIB MESYLATE:Pain in extremity:0.41), (DANTROLENE SODIUM:Pain in extremity:0.44), (DAPAGLIFLOZIN PROPANEDIOLMETFORMIN HYDROCHLORIDE:Pain in extremity:0.13), (DEFLAZACORT:Pain in extremity:1.67), (DESONIDE:Pain in extremity:1.14), (DESVENLAFAXINE:Pain in extremity:0.09), (DEXAMETHASONE ACETATE:Pain in extremity:0.89), (DEXTROAMPHETAMINE SULFATE:Pain in extremity:0.7), (DIGITOXIN:Pain in extremity:1.04), (DOBUTAMINE HYDROCHLORIDE:Pain in extremity:0.02), (DOLASETRON MESYLATE:Pain in extremity:0.83), (DOXYLAMINE SUCCINATE:Pain in extremity:0.47), (ECONAZOLE:Pain in extremity:1.05), (EFINACONAZOLE:Pain in extremity:0.17), (ELBASVIRGRAZOPREVIR:Pain in extremity:0.37), (ELOTUZUMAB:Pain in extremity:0.76), (ELTROMBOPAG OLAMINE:Pain in extremity:0.92), (EMPAGLIFLOZINLINAGLIPTIN:Pain in extremity:0.98), (ENALAPRIL MALEATELERCANIDIPINE HYDROCHLORIDE:Pain in extremity:0.1), (EPINASTINE HYDROCHLORIDE:Pain in extremity:1.17), (EPIRUBICIN HYDROCHLORIDE:Pain in extremity:0.19), (ERIBULIN:Pain in extremity:0.4), (ESTROGENS:Pain in extremity:0.96), (ETHINYL ESTRADIOLNORETHINDRONE:Pain in extremity:0.12), (ETHINYL ESTRADIOLNORGESTIMATE:Pain in extremity:0.33), (ETHINYL ESTRADIOLNORGESTREL:Pain in extremity:0.15), (ETRETINATE:Pain in extremity:0.71), (FENOTEROL HYDROBROMIDEIPRATROPIUM BROMIDE:Pain in extremity:0.57), (FERROUS BISGLYCINATE:Pain in extremity:0.22), (FERROUS SULFATE ANHYDROUS:Pain in extremity:0.06), (FERROUS SULFATEGLYCINE:Pain in extremity:0.33), (FLUOROMETHOLONE:Pain in extremity:0.35), (FLUTICASONE FUROATESALMETEROL XINAFOATE:Pain in extremity:1.63), (FORMOTEROL:Pain in extremity:1.2), (FOSFOMYCIN:Pain in extremity:0.12), (GIMERACILOTERACILTEGAFUR:Pain in extremity:1.02), (GLIMEPIRIDEROSIGLITAZONE MALEATE:Pain in extremity:0.2), (GLYCOPYRROLATEINDACATEROL MALEATE:Pain in extremity:0.46), (GLYCOPYRRONIUM:Pain in extremity:0.5), (GLYCOPYRRONIUMANDACATEROL:Pain in extremity:1.71), (GUANFACINE HYDROCHLORIDE:Pain in extremity:0.51), (HYDROCHLOROTHIAZIDEQUINAPRIL HYDROCHLORIDE:Pain in extremity:0.1), (HYDROCHLOROTHIAZIDESPIRONOLACTONE:Pain in extremity:0.28), (IDARUCIZUMAB:Pain in extremity:0.03), (ILOPROST:Pain in extremity:1.75), (IMIGLUCERASE:Pain in extremity:0.53), (IMIPENEM:Pain in extremity:0.03), (INDAPAMIDEPERINDOPRIL:Pain in extremity:2.16), (INTERFERON:Pain in extremity:0.98), (INTERFERON ALFA-2A:Pain in extremity:1.17), (INTERFERON ALFA-2B:Pain in extremity:0.91), (INTERFERON GAMMA-1B:Pain in extremity:0.13), (IRINOTECAN HYDROCHLORIDE:Pain in extremity:1.38), (LACTOBACILLUS RHAMNOSUS:Pain in extremity:1.01), (LAPATINIB DITOSYLATE:Pain in extremity:0.84), (LECITHIN:Pain in extremity:0.05), (LENVATINIB:Pain in extremity:0.37), (LERCANIDIPINE HYDROCHLORIDE:Pain in extremity:2.11), (LEVOTHYROXINE SODIUMPOTASSIUM IODIDE:Pain in extremity:0.37), (LORATADINEPSEUDOEPHEDRINE SULFATE:Pain in extremity:0.13), (LUTETIUM LU-177:Pain in extremity:0.13), (MAGNESIUM ASPARTATE HYDROCHLORIDE:Pain in extremity:0.27), (MAGNESIUM GLUCONATE:Pain in extremity:0.26), (MELPERONE:Pain in extremity:0.05), (METFORMIN HYDROCHLORIDEROSIGLITAZONE MALEATE:Pain in extremity:0.56), (METHENAMINE HIPPURATE:Pain in extremity:1.95), (METHENOLONE ENANTHATE:Pain in extremity:0.2), (METHOXY POLYETHYLENE GLYCOL-EPOETIN BETA:Pain in extremity:0.01), (MEXILETINE:Pain in extremity:0.09), (MOXONIDINE:Pain in extremity:0.96), (NATEGLINIDE:Pain in extremity:1.11), (OLMESARTAN:Pain in extremity:0.75), (ORPHENADRINE:Pain in extremity:0.95), (OXAPROZIN:Pain in extremity:0.3), (OXYBUTYNINOXYBUTYNIN CHLORIDE:Pain in extremity:0.41), (PANCRELIPASE:Pain in extremity:0.8), (PANITUMUMAB:Pain in extremity:0.79), (PARATHYROID HORMONE:Pain in extremity:0.31), (PEGINTERFERON ALFA-2B:Pain in extremity:0.23), (PENICILLIN V:Pain in extremity:0.9), (PENTAMIDINE ISETHIONATE:Pain in extremity:0.08), (PENTOSTATIN:Pain in extremity:0.09), (PHLOROGLUCINOL1,3,5-TRIMETHOXYBENZENE:Pain in extremity:0.09), (PILOCARPINE:Pain in extremity:0.9), (PILOCARPINE HYDROCHLORIDE:Pain in extremity:0.24), (PILSICAINIDE HYDROCHLORIDE:Pain in extremity:0.64), (PIMOBENDAN:Pain in extremity:0.26), (PNEUMOCOCCAL VACCINE, POLYVALENT 23:Pain in extremity:0.43), (POLYETHYLENE GLYCOL 3350POTASSIUM CHLORIDE\SODIUM BICARBONATE\SODIUM CHLORIDE\SODIUM SULFATE ANHYDROUS:Pain in extremity:0.45), (PRISTINAMYCIN:Pain in extremity:0.16), (PROTRIPTYLINE HYDROCHLORIDE:Pain in extremity:0.36), (QUINACRINE:Pain in extremity:0.03), (RAMUCIRUMAB:Pain in extremity:0.74), (RILPIVIRINE HYDROCHLORIDE:Pain in extremity:0.24), (RIOCIGUAT:Pain in extremity:0.77), (RIVASTIGMINE TARTRATE:Pain in extremity:1.61), (RUCAPARIB CAMSYLATE:Pain in extremity:0.26), (SALMETEROL:Pain in extremity:0.84), (SELEGILINE HYDROCHLORIDE:Pain in extremity:0.05), (SODIUM FLUORIDE:Pain in extremity:0.81), (SOTALOL HYDROCHLORIDE:Pain in extremity:0.57), (TASIMELTEON:Pain in extremity:0.11), (TELOTRISTAT ETHYL:Pain in extremity:0.33), (TEMSIROLIMUS:Pain in extremity:0.24), (TERBUTALINE:Pain in extremity:0.63), (TERBUTALINE SULFATE:Pain in extremity:1.85), (TESTOSTERONE UNDECANOATE:Pain in extremity:0.67), (THALIDOMIDE:Pain in extremity:2.1), (THEOPHYLLINE:Pain in extremity:0.29), (TRAMETINIB DIMETHYL SULFOXIDE:Pain in extremity:0.32), (TREPROSTINIL DIOLAMINE:Pain in extremity:0.08), (TRIHEXYPHENIDYL:Pain in extremity:0.08), (TRIMIPRAMINE MALEATE:Pain in extremity:0.65), (TRIPTORELIN PAMOATE:Pain in extremity:0.49), (UBIQUINOL:Pain in extremity:0.03), (VALGANCICLOVIR HYDROCHLORIDE:Pain in extremity:1.56), (VARDENAFIL HYDROCHLORIDE TRIHYDRATE:Pain in extremity:0.5), (VINORELBINEVINORELBINE TARTRATE:Pain in extremity:0.25), (VISMODEGIB:Pain in extremity:0.13), (WARFARIN POTASSIUM:Pain in extremity:0.55), (YEAST:Pain in extremity:0.43), (ZALEPLON:Pain in extremity:0.07), (ZILEUTON:Pain in extremity:0.47), (ESOMEPRAZOLE MAGNESIUM:Pain in jaw:0.23), (.ALPHA.1-PROTEINASE INHIBITOR HUMAN:Pain management:0.14), (ACARBOSE:Pain management:0.61), (ACETAMINOPHENPROPOXYPHENE NAPSYLATE:Pain management:0.34), (ACETAZOLAMIDE:Pain management:1.12), (ADO-TRASTUZUMAB EMTANSINE:Pain management:1.19), (ALBIGLUTIDE:Pain management:0.21), (ALISKIREN HEMIFUMARATE:Pain management:0.28), (ALUMINUM HYDROXIDEDIMETHICONEMAGNESIUM HYDROXIDE:Pain management:0.52), (AMILORIDE HYDROCHLORIDEHYDROCHLOROTHIAZIDE:Pain management:0.01), (AMINO ACIDSDEXTROSEELECTROLYTES NOS:Pain management:0.43), (AMIODARONE HYDROCHLORIDE:Pain management:1.17), (AMISULPRIDE:Pain management:0.53), (AMLODIPINE BESYLATEHYDROCHLOROTHIAZIDEOLMESARTAN MEDOXOMIL:Pain management:0.29), (AMLODIPINEVALSARTAN:Pain management:0.34), (AMMONIUM LACTATE:Pain management:0), (ASPIRIN LYSINE:Pain management:0.45), (ATRACURIUM:Pain management:0.23), (ATROPINE SULFATE:Pain management:0), (AZELASTINE:Pain management:0.35), (BACITRACIN:Pain management:0.24), (BENAZEPRILBENAZEPRIL HYDROCHLORIDE:Pain management:0.97), (BENDAMUSTINE:Pain management:0.08), (BIMATOPROST:Pain management:0.41), (BRINZOLAMIDE:Pain management:0.59), (CALCIUM CITRATEVITAMIN D:Pain management:0.03), (CALCIUM\MAGNESIUM:Pain management:0.27), (CALCIUM\MAGNESIUM\ZINC:Pain management:0.09), (CANAGLIFLOZINMETFORMIN HYDROCHLORIDE:Pain management:0.65), (CAPTOPRIL:Pain management:0.79), (CARVEDILOL PHOSPHATE:Pain management:1.29), (CASANTHRANOLDOCUSATE SODIUM:Pain management:0.06), (CASPOFUNGIN:Pain management:0.05), (CEFUROXIME AXETIL:Pain management:0.12), (CERITINIB:Pain management:0.08), (CETIRIZINE HYDROCHLORIDEPSEUDOEPHEDRINE HYDROCHLORIDE:Pain management:0.3), (CETUXIMAB:Pain management:1.25), (CISATRACURIUM:Pain management:0.04), (CLOZAPINE:Pain management:1.25), (CODEINE PHOSPHATEGUAIFENESIN:Pain management:0.12), (DACLIZUMAB:Pain management:1.05), (DANAZOL:Pain management:0.42), (DANTROLENE SODIUM:Pain management:0.13), (DAPAGLIFLOZIN PROPANEDIOL:Pain management:0.58), (DEFLAZACORT:Pain management:0.67), (DIGITOXIN:Pain management:0.15), (DOLASETRON MESYLATE:Pain management:0.25), (DOXEPIN HYDROCHLORIDE:Pain management:0.47), (DOXYLAMINE SUCCINATE:Pain management:0.17), (ECONAZOLE:Pain management:0.26), (EMPAGLIFLOZINLINAGLIPTIN:Pain management:0.77), (EPIRUBICIN:Pain management:1.02), (ERLOTINIB:Pain management:0.05), (ERYTHROPOIETIN:Pain management:1.36), (ESMOLOL HYDROCHLORIDE:Pain management:0.02), (ESOMEPRAZOLE MAGNESIUMNAPROXEN:Pain management:0.52), (ESTRADIOLNORETHINDRONE ACETATE:Pain management:0.09), (FISH OIL\OMEGA-3 FATTY ACIDS:Pain management:0.44), (FLUTICASONE FUROATE:Pain management:0.71), (FLUTICASONE FUROATESALMETEROL XINAFOATE:Pain management:0.78), (FLUTICASONE FUROATEVILANTEROL TRIFENATATE:Pain management:1.18), (FLUVASTATIN SODIUM:Pain management:0.01), (FORMOTEROL:Pain management:0.34), (FORMOTEROL FUMARATE:Pain management:0.64), (FORMOTEROL FUMARATE\MOMETASONE FUROATE:Pain management:0.05), (FUSIDIC ACID:Pain management:1.04), (GIMERACILOTERACILTEGAFUR:Pain management:1.07), (GLYCOPYRRONIUMINDACATEROL:Pain management:0.47), (GUANFACINE:Pain management:0.39), (HERBALSTURMERIC:Pain management:1), (HYALURONATE SODIUM:Pain management:0.34), (ILOPROST:Pain management:0.44), (IMIPENEM:Pain management:0.11), (IMIPRAMINE HYDROCHLORIDE:Pain management:1.2), (INDAPAMIDEPERINDOPRIL:Pain management:0.61), (INFLIXIMAB-DYYB:Pain management:0.04), (INTERFERON:Pain management:0.02), (INTERFERON ALFA-2A:Pain management:0.08), (INTERFERON ALFA-2B:Pain management:0.05), (IODINE:Pain management:0.09), (IPRATROPIUM:Pain management:1.66), (ISOFLURANE:Pain management:0.66), (LACOSAMIDE:Pain management:0.84), (LACTASE:Pain management:0.44), (LEDIPASVIRSOFOSBUVIR:Pain management:1.06), (LERCANIDIPINE:Pain management:1.18), (LERCANIDIPINE HYDROCHLORIDE:Pain management:0.67), (METHENAMINE HIPPURATE:Pain management:1.38), (MIDODRINE HYDROCHLORIDE:Pain management:0.18), (NADROPARIN CALCIUM:Pain management:0.42), (NALOXONE HYDROCHLORIDETILIDINE HYDROCHLORIDE:Pain management:0.03), (NINTEDANIB:Pain management:0.53), (NIRAPARIB:Pain management:0.37), (OMEPRAZOLE SODIUM:Pain management:0.13), (ORPHENADRINE:Pain management:0.06), (OSELTAMIVIR:Pain management:0.06), (OSPEMIFENE:Pain management:0.2), (PANTOPRAZOLE MAGNESIUM:Pain management:1.41), (PAZOPANIB HYDROCHLORIDE:Pain management:1.21), (PEGINTERFERON BETA-1A:Pain management:0.71), (PEMETREXED DISODIUM:Pain management:0.86), (PENTOSAN POLYSULFATE SODIUM:Pain management:0.38), (PERTUZUMAB:Pain management:0.4), (PILOCARPINE:Pain management:0.02), (PIOGLITAZONE:Pain management:0.06), (PIROXICAM:Pain management:0.45), (PITAVASTATIN CALCIUM:Pain management:1.1), (PRAZOSIN HYDROCHLORIDE:Pain management:0.36), (PREDNISOLONE ACETATE:Pain management:0.74), (PREDNISOLONE SODIUM PHOSPHATE:Pain management:0.03), (PROPAFENONE HYDROCHLORIDE:Pain management:0.18), (RABEPRAZOLE:Pain management:1), (RADIATION THERAPY:Pain management:0.84), (REGORAFENIB:Pain management:0.94), (RIVASTIGMINE:Pain management:0.42), (RIZATRIPTAN:Pain management:0.22), (SACUBITRILVALSARTAN:Pain management:0.63), (SALMETEROL XINAFOATE:Pain management:0.08), (SELEXIPAG:Pain management:0.12), (SUCCINYLCHOLINE CHLORIDE:Pain management:0.22), (SUFENTANIL CITRATE:Pain management:0.17), (SUMATRIPTANSUMATRIPTAN SUCCINATE:Pain management:1.86), (TAMOXIFEN CITRATE:Pain management:0.3), (TEDUGLUTIDE\WATER:Pain management:0.08), (TERAZOSIN HYDROCHLORIDE:Pain management:0.26), (TERBUTALINE SULFATE:Pain management:0.9), (TETRACYCLINE:Pain management:1.4), (TIMOLOL:Pain management:1.12), (TINZAPARIN:Pain management:0.28), (TIOTROPIUM:Pain management:0.67), (TOBRAMYCIN:Pain management:0.3), (TRANDOLAPRIL:Pain management:0.1), (TRASTUZUMAB:Pain management:0.33), (TROSPIUM CHLORIDE:Pain management:0.11), (UMECLIDINIUM BROMIDE:Pain management:0.22), (VALGANCICLOVIR HYDROCHLORIDE:Pain management:0.72), (VECURONIUM BROMIDE:Pain management:0.61), (VINORELBINEVINORELBINE TARTRATE:Pain management:0.03), (ACARBOSE:Pain prophylaxis:0.32), (ACETAMINOPHENBUTALBITALCAFFEINE:Pain prophylaxis:0.46), (ACETAMINOPHENOXYCODONEOXYCODONE HYDROCHLORIDE:Pain prophylaxis:0.32), (AGALSIDASE BETA:Pain prophylaxis:0.04), (AMIKACIN SULFATE:Pain prophylaxis:0.16), (AMISULPRIDE:Pain prophylaxis:0.38), (AMPICILLIN:Pain prophylaxis:0.61), (ATOVAQUONE:Pain prophylaxis:0.15), (AXITINIB:Pain prophylaxis:0.13), (BENAZEPRIL HYDROCHLORIDE:Pain prophylaxis:0.85), (BENAZEPRILBENAZEPRIL HYDROCHLORIDE:Pain prophylaxis:0.29), (BLINATUMOMAB:Pain prophylaxis:0.6), (BRINZOLAMIDE:Pain prophylaxis:0.25), (BUPRENORPHINE HYDROCHLORIDE:Pain prophylaxis:0.15), (CABAZITAXEL:Pain prophylaxis:0.07), (CALCITONIN:Pain prophylaxis:0.11), (CALCITONIN SALMON:Pain prophylaxis:0.47), (CALCIUM CARBONATECHOLECALCIFEROLMAGNESIUM CARBONATE:Pain prophylaxis:0.21), (CALCIUM CITRATE:Pain prophylaxis:0.31), (CANAKINUMAB:Pain prophylaxis:0.07), (CANDESARTAN CILEXETILHYDROCHLOROTHIAZIDE:Pain prophylaxis:1.46), (CARVEDILOL PHOSPHATE:Pain prophylaxis:0.33), (CHLORTHALIDONE:Pain prophylaxis:0.35), (CLINDAMYCIN PHOSPHATE:Pain prophylaxis:0), (CLINDAMYCINCLINDAMYCIN PHOSPHATE:Pain prophylaxis:1.9), (CORTISONEHYDROCORTISONE:Pain prophylaxis:0.71), (DABRAFENIB MESYLATE:Pain prophylaxis:0.02), (DACLIZUMAB:Pain prophylaxis:0.19), (DALFAMPRIDINE:Pain prophylaxis:0.18), (DALTEPARIN:Pain prophylaxis:0.82), (DANAZOL:Pain prophylaxis:0.35), (DASATINIB:Pain prophylaxis:1.59), (DEXMEDETOMIDINE HYDROCHLORIDE:Pain prophylaxis:0.92), (DIPYRIDAMOLE:Pain prophylaxis:0.41), (DOBUTAMINE HYDROCHLORIDE:Pain prophylaxis:0.01), (DOFETILIDE:Pain prophylaxis:0.28), (DOLASETRON MESYLATE:Pain prophylaxis:0.03), (DROPERIDOL:Pain prophylaxis:0.13), (ECONAZOLE:Pain prophylaxis:0.78), (EDOXABAN TOSYLATE:Pain prophylaxis:0.53), (ELOTUZUMAB:Pain prophylaxis:0.37), (EPIRUBICIN:Pain prophylaxis:1.01), (EPLERENONE:Pain prophylaxis:0.48), (ERIBULIN:Pain prophylaxis:0.38), (ERLOTINIB:Pain prophylaxis:0.35), (FELODIPINE:Pain prophylaxis:0.01), (FERROUS FUMARATE:Pain prophylaxis:0.04), (FESOTERODINE FUMARATE:Pain prophylaxis:0.56), (FLECAINIDE:Pain prophylaxis:1.24), (FLUOCINONIDE:Pain prophylaxis:0.15), (FLUOXETINEFLUOXETINE HYDROCHLORIDE:Pain prophylaxis:0.43), (FLUTICASONE:Pain prophylaxis:0.67), (FORMOTEROL FUMARATE:Pain prophylaxis:0.4), (FORMOTEROL FUMARATE DIHYDRATE\MOMETASONE FUROATE:Pain prophylaxis:0.34), (FOSPHENYTOINFOSPHENYTOIN SODIUM:Pain prophylaxis:0.28), (FUSIDIC ACID:Pain prophylaxis:0.27), (GLYBURIDE:Pain prophylaxis:0.76), (GLYCOPYRRONIUMINDACATEROL:Pain prophylaxis:0.65), (HYDROCHLOROTHIAZIDETELMISARTAN:Pain prophylaxis:0.19), (IBRITUMOMAB TIUXETAN:Pain prophylaxis:0.63), (IMATINIB MESYLATE:Pain prophylaxis:1.51), (IMIPENEM:Pain prophylaxis:0.32), (IMIPRAMINE HYDROCHLORIDE:Pain prophylaxis:0.29), (INDAPAMIDEPERINDOPRIL:Pain prophylaxis:1.2), (INOTUZUMAB OZOGAMICIN:Pain prophylaxis:0.18), (INTERFERON ALFA-2A:Pain prophylaxis:0.1), (INTERFERON ALFA-2B:Pain prophylaxis:0.36), (LABETALOL HYDROCHLORIDE:Pain prophylaxis:0.35), (LEDIPASVIRSOFOSBUVIR:Pain prophylaxis:0.72), (LENVATINIB:Pain prophylaxis:0.09), (LEVALBUTEROL HYDROCHLORIDE:Pain prophylaxis:0.53), (LEVOBUPIVACAINE:Pain prophylaxis:0.02), (LEVOLEUCOVORIN CALCIUM:Pain prophylaxis:0.55), (LITHIUM CARBONATE:Pain prophylaxis:0.67), (LOVASTATIN:Pain prophylaxis:1.1), (MECLIZINE HYDROCHLORIDE:Pain prophylaxis:0), (METHENAMINE HIPPURATE:Pain prophylaxis:0.35), (METHYLPREDNISOLONE ACETATE:Pain prophylaxis:0.27), (MUPIROCIN:Pain prophylaxis:0.02), (NEBIVOLOL HYDROCHLORIDE:Pain prophylaxis:0.67), (OLODATEROL HYDROCHLORIDETIOTROPIUM BROMIDE MONOHYDRATE:Pain prophylaxis:0.37), (OMEPRAZOLE SODIUM:Pain prophylaxis:0.19), (OSELTAMIVIR:Pain prophylaxis:0.8), (OXYBUTYNIN CHLORIDE:Pain prophylaxis:0.25), (PANTOPRAZOLE MAGNESIUM:Pain prophylaxis:1.06), (PAROXETINE:Pain prophylaxis:0.13), (PENICILLIN:Pain prophylaxis:0.79), (PENTOSTATIN:Pain prophylaxis:0.07), (PHENYTOIN SODIUM:Pain prophylaxis:0.43), (PIPERACILLIN:Pain prophylaxis:0), (PIROXICAM:Pain prophylaxis:0.07), (PRAMIPEXOLE DIHYDROCHLORIDE:Pain prophylaxis:0.37), (PRIMIDONE:Pain prophylaxis:0.89), (PYRAZINAMIDE:Pain prophylaxis:0.71), (RADIATION THERAPY:Pain prophylaxis:0.68), (RIZATRIPTAN BENZOATE:Pain prophylaxis:0.21), (ROSUVASTATIN:Pain prophylaxis:0.4), (SECUKINUMAB:Pain prophylaxis:0.44), (SITAGLIPTIN:Pain prophylaxis:0.66), (SODIUM CITRATE:Pain prophylaxis:0.06), (TAZOBACTAM:Pain prophylaxis:0.21), (TERAZOSIN HYDROCHLORIDE:Pain prophylaxis:0.05), (TERBINAFINE HYDROCHLORIDE:Pain prophylaxis:0.11), (TERBUTALINE SULFATE:Pain prophylaxis:0.1), (TERIFLUNOMIDE:Pain prophylaxis:0.04), (TETRACYCLINE:Pain prophylaxis:0.22), (TIGECYCLINE:Pain prophylaxis:0.13), (TIMOLOL:Pain prophylaxis:0.59), (TINZAPARIN:Pain prophylaxis:0.47), (TIZANIDINE HYDROCHLORIDE:Pain prophylaxis:0.54), (TOCILIZUMAB:Pain prophylaxis:0.39), (TOFACITINIB CITRATE:Pain prophylaxis:0.07), (TOLTERODINE TARTRATE:Pain prophylaxis:0.16), (TRIAMTERENE:Pain prophylaxis:0.01), (UMECLIDINIUM BROMIDEVILANTEROL TRIFENATATE:Pain prophylaxis:0.16), (VALGANCICLOVIR HYDROCHLORIDE:Pain prophylaxis:0.84), (VASOPRESSIN:Pain prophylaxis:0.01), (VECURONIUM BROMIDE:Pain prophylaxis:1.28), (VEMURAFENIB:Pain prophylaxis:0.7), (VINORELBINE TARTRATE:Pain prophylaxis:0.5), (VITAMIN B:Pain prophylaxis:0.8), (ZONISAMIDE:Pain prophylaxis:0.16), (LEUCOVORIN CALCIUM:Palliative care:0.24), (NILOTINIB:Palliative care:0.09), (RAMIPRIL:Palliative care:0.08), (VINCRISTINE SULFATE:Palliative care:0.75), (ANASTROZOLE:Palmar-plantar erythrodysaesthesia syndrome:0.38), (CALCIUM:Palmar-plantar erythrodysaesthesia syndrome:0.31), (LEVOLEUCOVORIN CALCIUM:Palmar-plantar erythrodysaesthesia syndrome:0.47), (METHYLPREDNISOLONE SODIUM SUCCINATE:Palmar-plantar erythrodysaesthesia syndrome:0.12), (NAPROXEN:Palmar-plantar erythrodysaesthesia syndrome:0), (PAZOPANIB HYDROCHLORIDE:Palmar-plantar erythrodysaesthesia syndrome:0.09), (ACETAMINOPHENOXYCODONEOXYCODONE HYDROCHLORIDE:Palpitations:0.12), (ACETAZOLAMIDE:Palpitations:0.03), (ALCOHOL:P alpitations:0.29), (BENZONATATE:Palpitations:0.22), (BISACODYL OR DOCUSATE SODIUM:Palpitations:0.73), (BORTEZOMIB:Palpitations:0.27), (BUMETANIDE:Palpitations:1.25), (CALCIUM ACETATE:Palpitations:0.33), (CALCIUM CARBONATEVITAMIN D:Palpitations:0.56), (CARVEDILOL PHOSPHATE:Palpitations:0.7), (CETIRIZINE HYDROCHLORIDEPSEUDOEPHEDRINE HYDROCHLORIDE:Palpitations:0.07), (COLESEVELAM HYDROCHLORIDE:Palpitations:0.36), (CORTISONEHYDROCORTISONE:Palpitations:0.67), (DABIGATRAN:Palpitations:0.12), (DACLIZUMAB:Palpitations:0.32), (DALFAMPRIDINE:P alpitations:0.79), (DAPAGLIFLOZIN PROPANEDIOL:Palpitations:0.35), (DASATINIB:Palpitations:0.9), (DIMENHYDRINATE:Palp itations:0.35), (DOXEPIN HYDROCHLORIDE:Palpitations:0.13), (DRONABINOL:Palpitations:0.21), (EMPAGLIFLOZI NLINAGLIPTIN:Palpitations:0.13), (EPLERENONE:Palpitations:0.08), (ESTROGENS, CONJUGATEDMEDROXYPROGESTERONE ACETATE:Palpitations:0.7), (FESOTERODINE FUMARATE:Palpitations:0.47), (FLUTICASONE FUROATEVILANTEROL TRIFENATATE:Palpitations:0.9), (FORMOTEROL FUMARATE:Palpitations:0.12), (HALOPERIDOL:Palpitations:0.12), (HERBALSTURMERIC: Palpitations:0.43), (HYDROCHLOROTHIAZIDETELMISARTAN:Palpitations:0.1), (HYDRO MORPHONE:Palpitations:0.5), (HYDROXYCHLOROQUINE DIPHOSPHATE:Palpitations:0.28), (HYOSCYAMINE:Palpitations:0.47), (IMATINIB MESYLATE:Palpitations:0.61), (INDAPAMIDE:Palpitations:0.58), (INDAPAMIDEPERINDO PRIL:Palpitations:0.36), (INFLUENZA VIRUS VACCINE:Palpitations:0.33), (INSULIN DEGLUDEC:Palpitations:0.04), (IPRATROPIUM:Palpitations:0.72), (LABETALOLLABETALOL HYDROCHLORIDE:Palpitations:0.72), (LEDIPASVIRSOFOSBUVIR:Palpitations:0.74), (LER CANIDIPINE:Palpitations:0.65), (LERCANIDIPINE HYDROCHLORIDE:Palpitations:0.64), (MEGESTROL ACETATE:Palpitations:0.01), (METHENAMINE HIPPURATE:Palpitations:0.16), (MIDODRINE:Palpitations:0.06), (MOXIFLOXACIN HYDROCHLORIDE:Palpitations:0.6), (NALOXONE HYDROCHLORIDEOXYCODONE HYDROCHLORIDE:Palpitations:0.55), (NINTEDANIB:Palpitations:0.18), (ONABOTULINUM TOXINA:Palpitations:0.08), (PANTOPRAZOLE MAGNESIUM:Palpitations:0.84), (PAROXETINEPAROXETINE HYDROCHLORIDE:Palpitations:0.8), (PAZOPANIB HYDROCHLORIDE:Palpitations:0.18), (PEGFILGRASTIM:Palpitations:0.18), (PHENOBARBI TAL:Palpitations:0.56), (POLYETHYLENE GLYCOLS:Palpitations:1.11), (PONATINIB:Palpitations:0.37), (PRAMIPEXOLE:Palpitations:0.66), (PYRIDOXINE:Palpitations:0.21), (QUETIAPINE:Palpitations:0.87), (RABEPRAZOLE:Pal pitations:0.39), (SCOPOLAMINE:Palpitations:0.08), (SELEXIPAG:Palpitations:0.66), (SULFAM ETHOXAZOLE:Palpitations:0.2), (TERBINAFINE HYDROCHLORIDE:Palpitations:0.07), (TERBUTALINE SULFATE:Palpitations:0.15), (TETRACYCLINE:Palpitations:0.72), (TOCOPHEROL:Palpitatio ns:0.02), (VITAMIN A:Palpitations:0.09), (ZINC:Palpitations:0.31), (ACETAMINOPHENOXYCODONEOXYCOD ONE HYDROCHLORIDE:Pancreatic carcinoma:0.36), (ADO-TRASTUZUMAB EMTANSINE:Pancreatic carcinoma:1.63), (ALCOHOL:Pancreatic carcinoma:0.09), (ALDESLEUKIN:Pancreatic carcinoma:0.23), (ALENDRONIC ACID:Pancreatic carcinoma:0.15), (ALFACALCIDOL:Pancreatic carcinoma:0.29), (AMINO ACIDSDEXTROSEELECTROLYTES NOS:Pancreatic carcinoma:0.44), (AMISULPRIDE:Pancreatic carcinoma:0.08), (AMLODIPINE BESYLATEHYDROCHLOROTHIAZIDEOLMESARTAN MEDOXOMIL:Pancreatic carcinoma:0.38), (ARMODAFINIL:Pancreatic carcinoma:0.35), (BENAZEPRILBENAZEPRIL HYDROCHLORIDE:Pancreatic carcinoma:0.48), (BETAMETHASONE VALERATE:Pancreatic carcinoma:0), (BIMATOPROST:Pancreatic carcinoma:0.03), (BUDESONIDEFORMOTEROL FUMARATE:Pancreatic carcinoma:0.1), (BUPRENORPHINE HYDROCHLORIDE:Pancreatic carcinoma:0.29), (BUPRENORPHINE HYDROCHLORIDENALOXONE HYDROCHLORIDE:Pancreatic carcinoma:0.11), (CALCITONIN:Pancreatic carcinoma:0.07), (CALCIUM CITRATE:Pancreatic carcinoma:0.58), (CANAGLIFLOZIN:Pancreatic carcinoma:0.12), (CANAGLIFLOZINMETFORMIN HYDROCHLORIDE:Pancreatic carcinoma:0.48), (CANAKINUMAB:Pancreatic carcinoma:0.18), (CANDESARTAN CILEXETILHYDROCHLOROTHIAZIDE:Pancreatic carcinoma:1.28), (CARFILZOMIB:Pancreatic carcinoma:0.71), (CARVEDILOL PHOSPHATE:Pancreatic carcinoma:0.68), (CERITINIB:Pancreatic carcinoma:0.61), (CERTOLIZUMAB PEGOL:Pancreatic carcinoma:1.31), (CHLORDIAZEPOXIDE HYDROCHLORIDECLIDINIUM BROMIDE:Pancreatic carcinoma:0.03), (CILASTATIN SODIUMIMIPENEM:Pancreatic carcinoma:0.45), (CILOSTAZOL:Pancreatic carcinoma:0.29), (DABIGATRAN:Pancreatic carcinoma:0.43), (DABRAFENIB:Pancreatic carcinoma:1.13), (DANAZOL:Pancreatic carcinoma:0.15), (DAPAGLIFLOZIN PROPANEDIOL:Pancreatic carcinoma:0), (DESLORATADINE:Pancreatic carcinoma:0.42), (DIVALPROEX SODIUM:Pancreatic carcinoma:0.92), (DOFETILIDE:Pancreatic carcinoma:0.43), (DOXEPIN HYDROCHLORIDE:Pancreatic carcinoma:0.04), (DRONEDARONE:Pancreatic carcinoma:0.08), (EDOXABAN TOSYLATE:Pancreatic carcinoma:0.88), (ELETRIPTAN HYDROBROMIDE:Pancreatic carcinoma:0.35), (EMPAGLIFLOZINLINAGLIPTIN:Pancreatic carcinoma:0.03), (EPLERENONE:Pancreatic carcinoma:0.31), (ERIBULIN:Pancreatic carcinoma:0.76), (ERIBULIN MESYLATE:Pancreatic carcinoma:0.04), (ESOMEPRAZOLE MAGNESIUMNAPROXEN:Pancreatic carcinoma:0.07), (ESZOPICLONE:Pancreatic carcinoma:0.96), (EVOLOCUMAB:Pancreatic carcinoma:0.57), (EXEMESTANE:Pancreatic carcinoma:1.31), (FEBUXOSTAT:Pancreatic carcinoma:0.53), (FESOTERODINE FUMARATE:Pancreatic carcinoma:0.57), (FLUOCINONIDE:Pancreatic carcinoma:0.42), (FLUTICASONE:Pancreatic carcinoma:0.65), (FLUTICASONE FUROATEVILANTEROL TRIFENATATE:Pancreatic carcinoma:0.23), (FORMOTEROL:Pancreatic carcinoma:0.18), (FORMOTEROL FUMARATE DIHYDRATE\MOMETASONE FUROATE:Pancreatic carcinoma:0.27), (FUSIDIC ACID:Pancreatic carcinoma:0.48), (GLYCOSAMINOGLYCANS:Pancreatic carcinoma:0.77), (HYDROXYUREA:Pancreatic carcinoma:1), (HYOSCYAMINE:Pancreatic carcinoma:0.37), (INDAPAMIDEPERINDOPRIL:Pancreatic carcinoma:1.11), (INTERFERON ALFA-2A:Pancreatic carcinoma:0.04), (IXAZOMIB:Pancreatic carcinoma:0.03), (LEDIPASVIRSOFOSBUVIR:Pancreatic carcinoma:0.54), (LEVALBUTEROL HYDROCHLORIDE:Pancreatic carcinoma:0.6), (LIDOCAINE HYDROCHLORIDE:Pancreatic carcinoma:0.56), (LISDEXAMFETAMINE DIMESYLATE:Pancreatic carcinoma:0.11), (LOMITAPIDE MESYLATE:Pancreatic carcinoma:0.09), (METHENAMINE HIPPURATE:Pancreatic carcinoma:0.28), (METHYLPREDNISOLONE ACETATE:Pancreatic carcinoma:0.32), (MODAFINIL:Pancreatic carcinoma:0.1), (MUPIROCINMUPIROCIN CALCIUM:Pancreatic carcinoma:0.01), (NABUMETONE:Pancreatic carcinoma:0.04), (NALOXONE:Pancreatic carcinoma:0.01), (NATEGLINIDE:Pancreatic carcinoma:0.16), (NILOTINIB:Pancreatic carcinoma:1.93), (NINTEDANIB:Pancreatic carcinoma:0.8), (NITROFURANTOIN:Pancreatic carcinoma:1.19), (OMEGA-3-ACID ETHYL ESTERS:Pancreatic carcinoma:1.01), (OMEPRAZOLE SODIUM:Pancreatic carcinoma:0.13), (OXCARBAZEPINE:Pancreatic carcinoma:0.64), (OXYBUTYNIN CHLORIDE:Pancreatic carcinoma:0.46), (PANTOPRAZOLE MAGNESIUM:Pancreatic carcinoma:1.25), (PENICILLIN:Pancreatic carcinoma:0.69), (PERTUZUMAB:Pancreatic carcinoma:0.8), (PHENOBARBITAL:Pancreatic carcinoma:0.3), (PONATINIB:Pancreatic carcinoma:0.85), (PRAMIPEXOLE DIHYDROCHLORIDE:Pancreatic carcinoma:0.52), (PROGESTERONE:Pancreatic carcinoma:0.21), (RAMUCIRUMAB:Pancreatic carcinoma:1.52), (RANOLAZINE:Pancreatic carcinoma:1.24), (REGORAFENIB:Pancreatic carcinoma:2.41), (RISEDRONATE SODIUM:Pancreatic carcinoma:0.45), (RIZATRIPTAN BENZOATE:Pancreatic carcinoma:0.29), (ROPINIROLE:Pancreatic carcinoma:0.04), (ROPINIROLE HYDROCHLORIDE:Pancreatic carcinoma:0.39), (SACUBITRILVALSARTAN:Pancreatic carcinoma:0.3), (SIPULEUCEL-T:Pancreatic carcinoma:0.06), (SODIUM OXYBATE:Pancreatic carcinoma:0.19), (SUMATRIPTANSUMATRIPTAN SUCCINATE:Pancreatic carcinoma:0.03), (TAMOXIFEN CITRATE:Pancreatic carcinoma:0.27), (TEDUGLUTIDE\WATER:Pancreatic carcinoma:0.3), (TERBINAFINE HYDROCHLORIDE:Pancreatic carcinoma:0.3), (TERBUTALINE SULFATE:Pancreatic carcinoma:0.47), (TETRACYCLINE:Pancreatic carcinoma:0.67), (THALIDOMIDE:Pancreatic carcinoma:0.74), (TIOTROPIUM:Pancreatic carcinoma:0.47), (TIZANIDINE HYDROCHLORIDE:Pancreatic carcinoma:0.69), (UMECLIDINIUM BROMIDE:Pancreatic carcinoma:0), (UMECLIDINIUM BROMIDEVILANTEROL TRIFENATATE:Pancreatic carcinoma:0.06), (VALGANCICLOVIR HYDROCHLORIDE:Pancreatic carcinoma:0.12), (VANCOMYCIN HYDROCHLORIDE:Pancreatic carcinoma:0.39), (ZINC:Pancreatic carcinoma:0.14), (ZIV-AFLIBERCEPT:Pancreatic carcinoma:0.85), (ADO-TRASTUZUMAB EMTANSINE:Pancreatic carcinoma metastatic:0.63), (BENAZEPRIL HYDROCHLORIDE:Pancreatic carcinoma metastatic:0.03), (BUDESONIDE:Pancreatic carcinoma metastatic:1.47), (BUMETANIDE:Pancreatic carcinoma metastatic:1.02), (CANDESARTAN CILEXETILHYDROCHLOROTHIAZIDE:Pancreatic carcinoma metastatic:0.8), (CARFILZOMIB:Pancreatic carcinoma metastatic:0.15), (CERTOLIZUMAB PEGOL:Pancreatic carcinoma metastatic:0.76), (CETUXIMAB:Pancreatic carcinoma metastatic:1.28), (CILASTATIN SODIUMIMIPENEM:Pancreatic carcinoma metastatic:0.17), (CYCLOBENZAPRINE:Pancreatic carcinoma metastatic:0.37), (DABRAFENIB:Pancreatic carcinoma metastatic:0.33), (DEVICE:Pancreatic carcinoma metastatic:0.12), (DOFETILIDE:Pancreatic carcinoma metastatic:0.04), (DOXAZOSIN MESYLATE:Pancreatic carcinoma metastatic:0.36), (EPINEPHRINE:Pancreatic carcinoma metastatic:0.49), (ESZOPICLONE:Pancreatic carcinoma metastatic:0.57), (EVOLOCUMAB:Pancreatic carcinoma metastatic:0.29), (EXEMESTANE:Pancreatic carcinoma metastatic:0.2), (FEBUXOSTAT:Pancreatic carcinoma metastatic:0.11), (FLUOXETINEFLUOXETINE HYDROCHLORIDE:Pancreatic carcinoma metastatic:0.05), (FLUTICASONE FUROATESALMETEROL XINAFOATE:Pancreatic carcinoma metastatic:0.07), (GLYCOSAMINOGLYCANS:Pancreatic carcinoma metastatic:0.2), (GUAIFENESIN:Pancreatic carcinoma metastatic:0.07), (IMATINIB MESYLATE:Pancreatic carcinoma metastatic:0.59), (INDAPAMIDEPERINDOPRIL:Pancreatic carcinoma metastatic:0.67), (ISOSORBIDE:Pancreatic carcinoma metastatic:0.41), (ISOSORBIDE MONONITRATE:Pancreatic carcinoma metastatic:0.66), (LABETALOLLABETALOL HYDROCHLORIDE:Pancreatic carcinoma metastatic:0.25), (LAPATINIB DITOSYLATE:Pancreatic carcinoma metastatic:0.1), (LEDIPASVIRSOFOSBUVIR:Pancreatic carcinoma metastatic:0.31), (LERCANIDIPINE HYDROCHLORIDE:Pancreatic carcinoma metastatic:0.66), (LITHIUM CARBONATE:Pancreatic carcinoma metastatic:0.12), (MECLIZINEMECLIZINE HYDROCHLORIDE:Pancreatic carcinoma metastatic:0.1), (NEBIVOLOL HYDROCHLORIDE:Pancreatic carcinoma metastatic:0.34), (NIACIN:Pancreatic carcinoma metastatic:0.08), (NILOTINIB:Pancreatic carcinoma metastatic:1.59), (OXCARBAZEPINE:Pancreatic carcinoma metastatic:0.16), (PAROXETINE:Pancreatic carcinoma metastatic:0.08), (PAZOPANIB HYDROCHLORIDE:Pancreatic carcinoma metastatic:0.95), (PEMETREXED:Pancreatic carcinoma metastatic:0.32), (PENICILLIN:Pancreatic carcinoma metastatic:0.05), (PERTUZUMAB:Pancreatic carcinoma metastatic:0.09), (PRIMIDONE:Pancreatic carcinoma metastatic:0.08), (QUETIAPINE FUMARATE:Pancreatic carcinoma metastatic:0.18), (RAMUCIRUMAB:Pancreatic carcinoma metastatic:0.44), (RANOLAZINE:Pancreatic carcinoma metastatic:0.61), (REGORAFENIB:Pancreatic carcinoma metastatic:0.88), (RISEDRONATE SODIUM:Pancreatic carcinoma metastatic:0.14), (SACUBITRILVALSARTAN:Pancreatic carcinoma metastatic:0.13), (SULFAMETHOXAZOLE:Pancreatic carcinoma metastatic:0.25), (TETRACYCLINE:Pancreatic carcinoma metastatic:0.12), (TIOTROPIUM:Pancreatic carcinoma metastatic:0.11), (TOPIRAMATE:Pancreatic carcinoma metastatic:0.69), (TRAMETINIB:Pancreatic carcinoma metastatic:0.48), (VANCOMYCIN HYDROCHLORIDE:Pancreatic carcinoma metastatic:0.11), (VEMURAFENIB:Pancreatic carcinoma metastatic:0.24), (ZIV-AFLIBERCEPT:Pancreatic carcinoma metastatic:0.24), (ANASTROZOLE:Pancreatic disorder:0.01), (BUMETANIDE:Pancreatic disorder:0.16), (CELECOXIB:Pancreatic disorder:0.41), (CERTOLIZUMAB PEGOL:Pancreatic disorder:0.05), (COLCHICINE:Pancreatic disorder:0.04), (DICLOFENAC:Pancreatic disorder:0.04), (DIGOXIN:Pancreatic disorder:0.66), (DILTIAZEM HYDROCHLORIDE:Pancreatic disorder:0.56), (EVOLOCUMAB:Pancreatic disorder:0.66), (FERROUS SULFATEFERROUS SULFATE, DRIED:Pancreatic disorder:0.43), (IRBESARTAN:Pancreatic disorder:0.14), (METRONIDAZOLE:Pancreatic disorder:0), (POLYETHYLENE GLYCOLS:Pancreatic disorder:0.1), (PRAVASTATIN:Pancreatic disorder:0.47), (RANOLAZINE:Pancreatic disorder:0.39), (SACUBITRILVALSARTAN:Pancreatic disorder:0.17), (TERIFLUNOMIDE:Pancreatic disorder:0.04), (WARFARIN SODIUM:Pancreatic disorder:0.24), (.ALPHA.1-PROTEINASE INHIBITOR HUMAN:Pancreatic insufficiency:0.11), (ACETAMINOPHENCODEINE PHOSPHATE:Pancreatic insufficiency:0.78), (ACETAMINOPHENOXYCODONE HYDROCHLORIDE:Pancreatic insufficiency:0.24), (AMIODARONE HYDROCHLORIDE:Pancreatic insufficiency:0.78), (ANASTROZOLE:Pancreatic insufficiency:0.5), (BECLOMETHASONE DIPROPIONATE:Pancreatic insufficiency:0.29), (BUPROPION:Pancreatic insufficiency:0.17), (CALCIUM ACETATE:Pancreatic insufficiency:0.2), (CANDESARTAN CILEXETILHYDROCHLOROTHIAZIDE:Pancreatic insufficiency:0.52), (CERTOLIZUMAB PEGOL:Pancreatic insufficiency:0.79), (CLINDAMYCINCLINDAMYCIN PHOSPHATE:Pancreatic insufficiency:0.53), (CLOTRIMAZOLE:Pancreatic insufficiency:0.27), (CORTISONEHYDROCORTISONE:Pancreatic insufficiency:0.07), (DASATINIB:Pancreatic insufficiency:0.62), (DEVICE:Pancreatic insufficiency:0.4), (DEXLANSOPRAZOLE:Pancreatic insufficiency:1.45), (DIPHENHYDRAMINE:Pancreatic insufficiency:0.56), (DUTASTERIDE:Pancreatic insufficiency:0.06), (ELETRIPTAN HYDROBROMIDE:Pancreatic insufficiency:0.1), (ENALAPRIL MALEATE:Pancreatic insufficiency:0.19), (EPLERENONE:Pancreatic insufficiency:0.06), (ESZOPICLONE:Pancreatic insufficiency:0.39), (EZETIMIBESIMVASTATIN:Pancreatic insufficiency:0.26), (FLUTICASONE:Pancreatic insufficiency:0.44), (FLUTICASONE FUROATEVILANTEROL TRIFENATATE:Pancreatic insufficiency:0.15), (FORMOTEROL FUMARATE:Pancreatic insufficiency:0.19), (FORMOTEROL FUMARATE\MOMETASONE FUROATE:Pancreatic insufficiency:0.23), (GLYBURIDE:Pancreatic insufficiency:0.25), (GLYCOPYRRONIUM:Pancreatic insufficiency:0.12), (HYDROCHLOROTHIAZIDEIRBESARTAN:Pancreatic insufficiency:0.23), (HYDROCHLOROTHIAZIDELISINOPRIL:Pancreatic insufficiency:0.42), (HYDROXYUREA:Pancreatic insufficiency:0.07), (IBRUTINIB:Pancreatic insufficiency:0.83), (INDAPAMIDEPERINDOPRIL:Pancreatic insufficiency:0.06), (IPRATROPIUM:Pancreatic insufficiency:0.23), (LERCANIDIPINE HYDROCHLORIDE:Pancreatic insufficiency:0.3), (LINAGLIPTIN:Pancreatic insufficiency:0.54), (LUMACAFTOR:Pancreatic insufficiency:0.33), (MAGNESIUM:Pancreatic insufficiency:0.53), (MEPOLIZUMAB:Pancreatic insufficiency:0.16), (METAXALONE:Pancreatic insufficiency:0.02), (METOPROLOL SUCCINATE:Pancreatic insufficiency:0.12), (METRONIDAZOLEMETRONIDAZOLE HYDROCHLORIDE:Pancreatic insufficiency:0.24), (MOXIFLOXACIN HYDROCHLORIDE:Pancreatic insufficiency:0.27), (NILOTINIB:Pancreatic insufficiency:0.34), (NITROFURANTOIN:Pancreatic insufficiency:0.32), (PANTOPRAZOLE:Pancreatic insufficiency:1.39), (PANTOPRAZOLE MAGNESIUM:Pancreatic insufficiency:1.05), (PAROXETINEPAROXETINE HYDROCHLORIDE:Pancreatic insufficiency:0.12), (PAZOPANIB HYDROCHLORIDE:Pancreatic insufficiency:0.16), (PONATINIB:Pancreatic insufficiency:0.11), (PRAMIPEXOLE DIHYDROCHLORIDE:Pancreatic insufficiency:0.28), (SACUBITRILVALSARTAN:Pancreatic insufficiency:0.42), (SECUKINUMAB:Pancreatic insufficiency:0.35), (SODIUM OXYBATE:Pancreatic insufficiency:0.59), (SULFAMETHOXAZOLE:Pancreatic insufficiency:0.04), (TETRACYCLINE:Pancreatic insufficiency:0.01), (TIOTROPIUM:Pancreatic insufficiency:0.03), (TOCILIZUMAB:Pancreatic insufficiency:0.11), (TOFACITINIB CITRATE:Pancreatic insufficiency:0.19), (UMECLIDINIUM BROMIDEVILANTEROL TRIFENATATE:Pancreatic insufficiency:0.35), (VARENICLINE TARTRATE:Pancreatic insufficiency:0.08), (ANASTROZOLE:Pancreatic neuroendocrine tumour:0.27), (BUDESONIDE:Pancreatic neuroendocrine tumour:0.4), (BUMETANIDE:Pancreatic neuroendocrine tumour:0.22), (BUPROPION HYDROCHLORIDE:Pancreatic neuroendocrine tumour:0.11), (CANDESARTAN:Pancreatic neuroendocrine tumour:0.04), (CHOLESTYRAMINE:Pancreatic neuroendocrine tumour:0.09), (CYCLOBENZAPRINE:Pancreatic neuroendocrine tumour:0.12), (DEVICE:Pancreatic neuroendocrine tumour:0.12), (DEXLANSOPRAZOLE:Pancreatic neuroendocrine tumour:0.32), (DICLOFENAC:Pancreatic neuroendocrine tumour:0.01), (DIGOXIN:Pancreatic neuroendocrine tumour:0.4), (DIPHENHYDRAMINE:Pancreatic neuroendocrine tumour:0.15), (DOCUSATE SODIUM:Pancreatic neuroendocrine tumour:0.2), (EVOLOCUMAB:Pancreatic neuroendocrine tumour:0.32), (IBRUTINIB:Pancreatic neuroendocrine tumour:0.1), (IMATINIB MESYLATE:Pancreatic neuroendocrine tumour:0.04), (LOVASTATIN:Pancreatic neuroendocrine tumour:0.1), (POLYETHYLENE GLYCOLS:Pancreatic neuroendocrine tumour:0.18), (TERIFLUNOMIDE:Pancreatic neuroendocrine tumour:0.03), (TIOTROPIUM BROMIDE MONOHYDRATE:Pancreatic neuroendocrine tumour:0.04), (TOPIRAMATE:Pancreatic neuroendocrine tumour:0.53), (VERAPAMIL HYDROCHLORIDE:Pancreatic neuroendocrine tumour:0.21), (ANASTROZOLE:Pancreatitis:0.03), (BUDESONIDE:Pancreatitis:1.25), (BUMET ANIDE:Pancreatitis:0.31), (BUPROPION:Pancreatitis:0.03), (CALCIUM CARBONATECHOLECALCIFEROL:Pancreatitis:0.6), (CANDESARTAN:Pancreatitis:0.04), (CEPHALEXIN:Pancreatitis:0.06), (DEVICE:Pancreatitis:0.29), (DEXLANSOPRAZOLE:Pancre atitis:1.03), (ENALAPRIL MALEATE:Pancreatitis:0.14), (ENOXAPARIN:Pancreatitis:0.45), (EVOLOCUMAB:Pancreatiti s:0.7), (HYDROCHLOROTHIAZIDELISINOPRIL:Pancreatitis:0.21), (IBRUTINIB:Pancreatitis: 1.11), (IRBESARTAN:Pancreatitis:0.1), (ISOSORBIDE:Pancreatitis:0.01), (LERCANIDIPINE:P ancreatitis:0.18), (LISDEXAMFETAMINE DIMESYLATE:Pancreatitis:0.04), (LORATADINE:Pancreatitis:0.41), (NADOLOL:Pancreatitis: 0), (NEBIVOLOL:Pancreatitis:0.13), (OXCARBAZEPINE:Pancreatitis:0.56), (PANTOPRAZOLE MAGNESIUM:Pancreatitis:0.05), (PHENOBARBITAL:Pancreatitis:0.02), (PRIMIDONE:Pancre atitis:0.09), (RANOLAZINE:Pancreatitis:0.39), (SACUBITRILVALSARTAN:Pancreatitis:0.01), (SECUKINUMAB:Pancreatitis:0.17), (SOTALOL:Pancreatitis:0.02), (TERIFLUNOMIDE:Pancr eatitis:0.18), (TOCILIZUMAB:Pancreatitis:0.59), (TOFACITINIB CITRATE:Pancreatitis:0.22), (TRAMADOL HYDROCHLORIDE:Pancreatitis:0.19), (VARENICLINE TARTRATE:Pancreatitis:0.3), (CALCIUM CARBONATECHOLECALCIFEROL:Pancreatitis chronic:0.17), (CYCLOBENZAPRINE:Pancreatitis chronic:0.03), (DEVICE:Pancreatitis chronic:0.31), (DILTIAZEM:Pancreatitis chronic:0.39), (DIVALPROEX SODIUM:Pancreatitis chronic:0.05), (EVEROLIMUS:Pancreatitis chronic:0.18), (EVOLOCUMAB:Pancreatitis chronic:0.42), (FERROUS SULFATE:Pancreatitis chronic:0.26), (ILOPROST:Pancreatitis chronic:0.15), (LOVASTATIN:Pancreatitis chronic:0.34), (MELOXICAM:Pancreatitis chronic:0.24), (NAPROXEN:Pancreatitis chronic:0.43), (NEBIVOLOL:Pancreatitis chronic:0.01), (PANTOPRAZOLE:Pancreatitis chronic:0.56), (PRAVASTATIN:Pancreatitis chronic:0.11), (RANOLAZINE:Pancreatitis chronic:0.19), (BENDAMUSTINE HYDROCHLORIDE:Pancytopenia:0.04), (BUMETANIDE:Pancytopenia:0.47), (CALCIUM CARBONATECHOLECALCIFEROL:Pancytopenia:0.14), (DILTIAZEM HYDROCHLORIDE:Pancytopenia:0.11), (ENALAPRIL:Pancytopenia:0.49), (ESCITALOPRA M OXALATE:Pancytopenia:0.24), (FERROUS SULFATE:Pancytopenia:0), (FERROUS SULFATEFERROUS SULFATE, DRIED:Pancytopenia:0.04), (IBRITUMOMAB TIUXETAN:Pancytopenia:0.16), (IMATINIB MESYLATE:Pancytopenia:0.14), (MICAFUNGIN:Pancytopenia:0.23), (MITOXANTRONE HYDROCHLORIDE:Pancytopenia:0.38), (NEBIVOLOL:Pancytopenia:0.16), (PANTOPRAZOL E:Pancytopenia:0.34), (PYRAZINAMIDE:Pancytopenia:0.65), (TORSEMIDE:Pancytopenia:0.54), (.ALPHA.1-PROTEINASE INHIBITOR HUMAN:Panic attack:0.74), (ACARBOSE:Panic attack:0.28), (ACETAMINOPHENDICHLORALPHENAZONEISOMETHEPTENE:Panic attack:0.07), (ACETAZOLAMIDE:Panic attack:0.76), (ALENDRONIC ACID:Panic attack:0.34), (AMANTADINE HYDROCHLORIDE:Panic attack:0.63), (AMIODARONE:Panic attack:1.25), (AMISULPRIDE:Panic attack:0.64), (AMLODIPINE BESYLATEHYDROCHLOROTHIAZIDEOLMESARTAN MEDOXOMIL:Panic attack:0.01), (AMMONIUM LACTATE:Panic attack:0.2), (BICALUTAMIDE:Panic attack:1.27), (BRINZOLAMIDE:Panic attack:0.21), (BUTYLSCOPOLAMINE BROMIDE:Panic attack:0.16), (CALCIPOTRIENE:Panic attack:0.08), (CALCIUM ACETATE:Panic attack:0.65), (CALCIUM CARBONATEVITAMIN D:Panic attack:0.93), (CANAGLIFLOZINMETFORMIN HYDROCHLORIDE:Panic attack:0.61), (CANAKINUMAB:Panic attack:0.29), (CANDESARTAN CILEXETILHYDROCHLOROTHIAZIDE:Panic attack:0.96), (CARFILZOMIB:Panic attack:0.19), (CEFTRIAXONE SODIUM:Panic attack:0.06), (CHLORDIAZEPOXIDE HYDROCHLORIDECLIDINIUM BROMIDE:Panic attack:0.4), (CHLORPHENIRAMINEHYDROCODONE:Panic attack:0.21), (CIMETIDINE:Panic attack:0.95), (CINACALCET HYDROCHLORIDE:Panic attack:0.43), (CLINDAMYCIN:Panic attack:0.63), (CORTICOTROPIN:Panic attack:0.6), (CYPROHEPTADINE:Panic attack:0.79), (DACLIZUMAB:Panic attack:0.84), (DASATINIB:Panic attack:0.84), (DEXTROAMPHETAMINE:Panic attack:0.22), (DOXYLAMINE SUCCINATE:Panic attack:0.04), (DUTASTERIDETAMSULOSIN HYDROCHLORIDE:Panic attack:0.14), (EMPAGLIFLOZINLINAGLIPTIN:Panic attack:0.71), (ENOXAPARIN:Panic attack:0.89), (EPLERENONE:Panic attack:0.3), (ERYTHROPOIETIN:Panic attack:0.25), (ETORICOXIB:Panic attack:0.26), (EVOLOCUMAB:Panic attack:2.01), (FEXOFENADINEPSEUDOEPHEDRINE:Panic attack:0.52), (FLUOCINONIDE:Panic attack:0.32), (FLUOXETINE HYDROCHLORIDEOLANZAPINE:Panic attack:0.17), (FLURAZEPAMFLURAZEPAM HYDROCHLORIDE:Panic attack:0.2), (FLUTICASONE FUROATESALMETEROL XINAFOATE:Panic attack:0.71), (FORMOTEROL FUMARATE\MOMETASONE FUROATE:Panic attack:0.69), (FROVATRIPTAN SUCCINATE:Panic attack:0.04), (FUSIDIC ACID:Panic attack:0.13), (GLYCOPYRROLATE:Panic attack:0.21), (GLYCOPYRRONIUM:Panic attack:0.18), (GLYCOPYRRONIUM1NDACATEROL:Panic attack:0.56), (HYDROCODONE BITARTRATEIBUPROFEN:Panic attack:0.36), (HYOSCYAMINE SULFATEMETHENAMINEMETHYLENE BLUEPHENYL SALICYLATE\SODIUM PHOSPHATE, MONOBASIC, MONOHYDRATE:Panic attack:0.01), (IBRUTINIB:Panic attack:0.5), (ILOPROST:Panic attack:0.37), (IMATINIB MESYLATE:Panic attack:0.01), (INDACATEROL:Panic attack:0.05), (LACTASE:Panic attack:0.43), (LACTOBACILLUS RHAMNOSUS:Panic attack:0.1), (LERCANIDIPINE:Panic attack:0.25), (LERCANIDIPINE HYDROCHLORIDE:Panic attack:0.11), (LEVOMEFOLATE CALCIUM\METHYLCOBALAMINPYRIDOXAL PHOSPHATE ANHYDROUS:Panic attack:0.11), (LOMITAPIDE MESYLATE:Panic attack:0.59), (MEGESTROL ACETATE:Panic attack:0.37), (MEPOLIZUMAB:Panic attack:0.52), (METHIMAZOLE:Panic attack:0.02), (MIDODRINE HYDROCHLORIDE:Panic attack:0.31), (MINOCYCLINEMINOCYCLINE HYDROCHLORIDE:Panic attack:0.34), (NALOXONE HYDROCHLORIDEOXYCODONE HYDROCHLORIDE:Panic attack:0.53), (NILOTINIB:Panic attack:0.65), (NORETHINDRONE:Panic attack:1.16), (OXYBUTYNINOXYBUTYNIN CHLORIDE:Panic attack:0.14), (PALIPERIDONE PALMITATE:Panic attack:0.88), (PAMIDRONATE DISODIUM:Panic attack:0.02), (PANTOPRAZOLE MAGNESIUM:Panic attack:1.4), (PAZOPANIB HYDROCHLORIDE:Panic attack:0.28), (PEGINTERFERON BETA-1A:Panic attack:0.89), (PENICILLIN:Panic attack:1.4), (PHENTERMINE HYDROCHLORIDE:Panic attack:0.27), (PILOCARPINE:Panic attack:0.03), (PONATINIB:Panic attack:0.31), (PROTRIPTYLINE HYDROCHLORIDE:Panic attack:0.62), (PYRAZINAMIDE:Panic attack:0.71), (QUINAPRIL:Panic attack:0.09), (RABEPRAZOLE:Panic attack:0.69), (RETINOL:Panic attack:0.02), (RIVASTIGMINE TARTRATE:Panic attack:0.67), (CEREVISIAE:Panic attack:0.04), (SALMETEROL XINAFOATE:Panic attack:0.48), (SILODOSIN:Panic attack:0.08), (SULFAMETHOXAZOLE:Panic attack:0.29), (TERBINAFINE HYDROCHLORIDE:Panic attack:0.52), (TOCILIZUMAB:Panic attack:0.46), (TOCOPHEROL:Panic attack:0.42), (ZILEUTON:Panic attack:0.11), (ALCOHOL:Panic disorder:0.45), (AMANTADINE:Panic disorder:0.18), (AMANTADINE HYDROCHLORIDE:Panic disorder:0.02), (AMISULPRIDE:Panic disorder:0.32), (ARMODAFINIL:Panic disorder:1.02), (AZITHROMYCIN DIHYDRATE:Panic disorder:0.16), (BENAZEPRIL HYDROCHLORIDE:Panic disorder:0.33), (BENZTROPINE MESYLATE:Panic disorder:0.01), (BISACODYL OR DOCUSATE SODIUM:Panic disorder:0.18), (BUPRENORPHINE HYDROCHLORIDE:Panic disorder:0.84), (CALCITRIOL:Panic disorder:0.14), (CALCIUM CARBONATECHOLECALCIFEROL:Panic disorder:1.42), (CALCIUM CITRATE:Panic disorder:0.06), (CARVEDILOL PHOSPHATE:Panic disorder:0.25), (CIPROFLOXACIN HYDROCHLORIDE:Panic disorder:0.6), (CLINDAMYCINCLINDAMYCIN PHOSPHATE:Panic disorder:0.26), (CLOTRIMAZOLE:Panic disorder:0.14), (COLCHICINE:Panic disorder:0.55), (DASATINIB:Panic disorder:0.57), (DESLORATADINE:Panic disorder:0.15), (DICLOFENAC:Panic disorder:1.17), (DOCUSATE SODIUM:Panic disorder:1.24), (DOXAZOSINDOXAZOSIN MESYLATE:Panic disorder:0.05), (DUTASTERIDE:Panic disorder:0.39), (ETORICOXIB:Panic disorder:0.26), (EVEROLIMUS:Panic disorder:0.52), (EVOLOCUMAB:Panic disorder:1.24), (FEBUXOSTAT:Panic disorder:0.02), (FESOTERODINE FUMARATE:Panic disorder:0.06), (FLUTICASONE FUROATEVILANTEROL TRIFENATATE:Panic disorder:0.28), (GUANFACINE:Panic disorder:0.36), (GUANFACINE HYDROCHLORIDE:Panic disorder:0.18), (HEPARIN SODIUM:Panic disorder:0.15), (HYDROMORPHONE:Panic disorder:0.09), (HYDROXYCHLOROQUINE DIPHOSPHATE:Panic disorder:0.22), (ILOPROST:Panic disorder:0), (INSULIN ASPART:Panic disorder:1.36), (INSULIN DETEMIR:Panic disorder:0.54), (IPRATROPIUM:Panic disorder:0.25), (ISOSORBIDE:Panic disorder:0.36), (LABETALOLLABETALOL HYDROCHLORIDE:Panic disorder:0.52), (LEDIPASVIRSOFOSBUVIR:Panic disorder:0.22), (LERCANIDIPINE:Panic disorder:0.26), (LIDOCAINEPRILOCAINE:Panic disorder:0), (LINAGLIPTIN:Panic disorder:0.41), (METAXALONE:Panic disorder:0.71), (METHYLPREDNISOLONE ACETATE:Panic disorder:0.24), (MOXIFLOXACIN HYDROCHLORIDE:Panic disorder:0.01), (NALOXONE HYDROCHLORIDEOXYCODONE HYDROCHLORIDE:Panic disorder:0.07), (NALTREXONE:Panic disorder:0.22), (NITROFURANTOIN:Panic disorder:0.39), (OXYBUTYNIN CHLORIDE:Panic disorder:0.21), (PAZOPANIB HYDROCHLORIDE:Panic disorder:0.01), (PEGINTERFERON BETA-1A:Panic disorder:0.15), (PENICILLIN:Panic disorder:0.29), (RABEPRAZOLE:Panic disorder:0.27), (RALOXIFENE HYDROCHLORIDE:Panic disorder:0.07), (RIZATRIPTAN BENZOATE:Panic disorder:0.4), (ROFLUMILAST:Panic disorder:0.05), (ROPINIROLE HYDROCHLORIDE:Panic disorder:0.67), (SACUBITRILVALSARTAN:Panic disorder:0.65), (SUMATRIPTANSUMATRIPTAN SUCCINATE:Panic disorder:0.46), (TERBUTALINE SULFATE:Panic disorder:0.11), (UMECLIDINIUM BROMIDEVILANTEROL TRIFENATATE:Panic disorder:0.31), (DEXLANSOPRAZOLE:Papillary thyroid cancer:0.02), (DIGOXIN:Papillary thyroid cancer:0.04), (EVEROLIMUS:Papillary thyroid cancer:0.26), (EVOLOCUMAB:Papillary thyroid cancer:0.26), (ABIRATERONE ACETATE:Paraesthesia:0.12), (ALENDRONIC ACID:Paraesthesia:0.03), (ANASTROZOLE:Paraesthesia:1.4), (AZITHROMYCIN DIHYDRATE:Paraesthesia:0.16), (BENAZEPRILBENAZEPRIL HYDROCHLORIDE:Paraesthesia:0.1), (BISACODYL OR DOCUSATE SODIUM:Paraesthesia:0.89), (BUDESONIDEFORMOTEROL FUMARATE:Paraesthesia:0.07), (BUPRENORPHINE HYDROCHLORIDE:Paraesthesia:0.09), (BUPRENORPHINE HYDROCHLORIDENALOXONE HYDROCHLORIDE:Paraesthesia:0.15), (CALCIUM CARBONATEVITAMIN D:Paraesthesia:0.11), (CALCIUM CITRATE:Paraesthesia:0.87), (CANAGLIFLOZINMETFORMIN HYDROCHLORIDE:Paraesthesia:0.12), (CANDESARTAN:Paraesthesia:1.08), (CARVEDILOL PHOSPHATE:Paraesthesia:0.5), (CETUXIMAB:Paraesthesia:0.15), (CLOZAPINE:Paraesthesia: 0.45), (CODEINE:Paraesthesia:0.34), (DACLIZUMAB:Paraesthesia:0.76), (DASATINIB:Paraest hesia:0.6), (DEVICE:Paraesthesia:0.33), (DIMENHYDRINATE:Paraesthesia:0.44), (DOFETILID E:Paraesthesia:0.18), (DRONABINOL:Paraesthesia:0.14), (DULAGLUTIDE:Paraesthesia:0.02), (EMPAGLIFLOZINLINAGLIPTIN:Paraesthesia:0.22), (ESZOPICLONE:Paraesthesia:1.07), (EV OLOCUMAB:Paraesthesia:1.23), (FISH OIL\OMEGA-3 FATTY ACIDS:Paraesthesia:0.05), (FLUTICASONE FUROATE:Paraesthesia:0.16), (HEPARIN SODIUM:Paraesthesia:0.5), (HERBALSTURMERIC:Paraesthesia:0.34), (HYDROCHLOROTH IAZIDEIRBESARTAN:Paraesthesia:0.34), (HYDROCHLOROTHIAZIDEVALSARTAN:Para esthesia:0.36), (HYDROXYCHLOROQUINE DIPHOSPHATE:Paraesthesia:0.51), (IMIPRAMINE HYDROCHLORIDE:Paraesthesia:0.19), (INDAPAMIDEPERINDOPRIL:Paraesthesia:0.25), (IP RATROPIUM:Paraesthesia:0.59), (ISOSORBIDE:Paraesthesia:0.65), (LABETALOLLABETAL OL HYDROCHLORIDE:Paraesthesia:0.44), (LEVALBUTEROL HYDROCHLORIDE:Paraesthesia:0.51), (LIDOCAINEPRILOCAINE:Paraesthesia:0.24), (MET HENAMINE HIPPURATE:Paraesthesia:0.36), (NABUMETONE:Paraesthesia:0.16), (NEBIVOLOL:Paraesthe sia:0.43), (NILOTINIB:Paraesthesia:1.01), (PANTOPRAZOLE MAGNESIUM:Paraesthesia:0.64), (PAZOPANIB HYDROCHLORIDE:Paraesthesia:0.36), (PHENOBARBITAL:Paraesthesia:0.06), (PITAVASTA TIN CALCIUM:Paraesthesia:0.18), (PRIMIDONE:Paraesthesia:0.76), (ROFLUMILAST:Paraesthesia: 0.42), (SACUBITRILVALSARTAN:Paraesthesia:1.1), (SCOPOLAMINE:Paraesthesia:0.3), (SE CUKINUMAB:Paraesthesia:0.73), (SULFAMETHOXAZOLE:Paraesthesia:0.18), (TICAGRELO R:Paraesthesia:0.04), (TIMOLOL:Paraesthesia:0.31), (TIOTROPIUM:Paraesthesia:0.08), (UMEC LIDINIUM BROMIDEVILANTEROL TRIFENATATE:Paraesthesia:0.2), (ATRACURIUM:Paralysis:0.49), (ROPIVACAINE:Paralysis: 0.43), (ALCOHOL:Paranoia:0.31), (BUPRENORPHINE HYDROCHLORIDE:Paranoia:0.03), (ESOMEPRAZOLE MAGNESIUM:Paranoia:0.16), (PAROXETINE:Paranoia:0.14), (VARENICLINE TARTRATE:Paranoia:0.67), (ZONISAMIDE:Paranoia:0.41), (.ALPHA.1-PROTEINASE INHIBITOR HUMAN:Parkinson's disease:0.6), (ACAMPROSATE:Parkinson's disease:0.5), (ACECLOFENAC:Parkinson's disease:0.08), (ACETAMINOPHENOXYCODONEOXYCODONE HYDROCHLORIDE:Parkinson's disease:1.88), (ACITRETIN:Parkinson's disease:0.64), (ACTIVATED CHARCOAL:Parkinson's disease:0.65), (ADO-TRASTUZUMAB EMTANSINE:Parkinson's disease:0.97), (AGALSIDASE BETA:Parkinson's disease:1.8), (ALEMTUZUMAB:Parkinson's disease:0.51), (ALUMINUM HYDROXIDEMAGNESIUM OXIDEOXETHAZAINE:Parkinson's disease:0.05), (AMBROXOLCLENBUTEROL:Parkinson's disease:0.25), (AMINOPHYLLINE:Parkinson's disease:0.13), (AMOBARBITAL SODIUM:Parkinson's disease:0.06), (AMOXICILLINCLARITHROMYCINESOMEPRAZOLE:Parkinson's disease:0.74), (AMPICILLIN SODIUM:Parkinson's disease:0.05), (ANHYDROUS CITRIC ACIDASPIRIN\SODIUM BICARBONATE:Parkinson's disease:0), (APREPITANT:Parkinson's disease:0.94), (ARGININE:Parkinson's disease:0.04), (ARIPIPRAZOLE LAUROXIL:Parkinson's disease:0.85), (ASENAPINE:Parkinson's disease:0.15), (ASPIRINBUTALBITALCAFFEINE:Parkinson's disease:0.07), (ATROPINEDIPHENOXYLATE:Parkinson's disease:0.37), (AVOCADO OILSOYBEAN OIL:Parkinson's disease:0.01), (AZITHROMYCIN OR METHOTREXATE SODIUM:Parkinson's disease:0.67), (BALSALAZIDE DISODIUM:Parkinson's disease:0.24), (BECLOMETHASONE:Parkinson's disease:0.71), (BECLOMETHASONE DIPROPIONATEFORMOTEROL:Parkinson's disease:0.42), (BENIDIPINE:Parkinson's disease:0.03), (BENZOCAINE:Parkinson's disease:0.06), (BEPOTASTINE:Parkinson's disease:0.06), (BETAMETHASONE DIPROPIONATECALCIPOTRIENE:Parkinson's disease:0.42), (BETAMETHASONECALCIPOTRIENE:Parkinson's disease:0.5), (BILASTINE:Parkinson's disease:0.92), (BIMATOPROSTTIMOLOL:Parkinson's disease:0.73), (BLONANSERIN:Parkinson's disease:0.99), (BOCEPREVIR:Parkinson's disease:0.06), (BOCOCIZUMAB:Parkinson's disease:0.3), (BROMPERIDOL:Parkinson's disease:0.24), (CABOZANTINIB S-MALATE:Parkinson's disease:0.09), (CAJUPUT OILEUCALYPTUS OILPEPPERMINT OIL:Parkinson's disease:0.01), (CALCIUM CARBONATEERGOCALCIFEROLRETINOL:Parkinson's disease:0.28), (CANAGLIFLOZINMETFORMIN HYDROCHLORIDE:Parkinson's disease:1.64), (CAPSAICIN:Parkinson's disease:0.35), (CARBOXYMETHYLCELLULOSE SODIUMGLYCERINPOLYSORBATE 80:Parkinson's disease:0.02), (CARVEDILOL PHOSPHATE:Parkinson's disease:2.6), (CEFEPIMECEFEPIME HYDROCHLORIDE:Parkinson's disease:0.46), (CEFPODOXIME PROXETIL:Parkinson's disease:0.44), (CERIVASTATIN SODIUM:Parkinson's disease:0.12), (CETUXIMAB:Parkinson's disease:0.78), (CHLORDIAZEPOXIDE HYDROCHLORIDECLIDINIUM BROMIDE:Parkinson's disease:0.87), (CHLOROTHIAZIDE:Parkinson's disease:0.11), (CHLORPHENIRAMINEHYDROCODONE:Parkinson's disease:0.37), (CHLORPROMAZINE HYDROCHLORIDE:Parkinson's disease:0.01), (CHLORPROTHIXENE:Parkinson's disease:0.17), (CLENBUTEROL HYDROCHLORIDE:Parkinson's disease:0.24), (CLIKSTAR:Parkinson's disease:0.65), (CLOXACILLIN SODIUM:Parkinson's disease:0.3), (COLCHICINEDICYCLOMINE HYDROCHLORIDE:Parkinson's disease:0.09), (CORTICOTROPIN:Parkinson's disease:0.92), (CYPROHEPTADINE HYDROCHLORIDE:Parkinson's disease:0.27), (DABRAFENIB MESYLATE:Parkinson's disease:0.11), (DACLIZUMAB:Parkinson's disease:1.25), (DAPAGLIFLOZIN PROPANEDIOLMETFORMIN HYDROCHLORIDE:Parkinson's disease:0.42), (DEFLAZACORT:Parkinson's disease:2.01), (DEGARELIX:Parkinson's disease:0.83), (DEXAMETHASONE ACETATE:Parkinson's disease:0.76), (DILAZEP:Parkinson's disease:0.03), (DIMETHICONESILICON DIOXIDESIMETHICONE:Parkinson's disease:0.21), (DIPHENIDOL HYDROCHLORIDE:Parkinson's disease:0.18), (DIPYRONEPROMETHAZINE HYDROCHLORIDE:Parkinson's disease:0.34), (DOLASETRON MESYLATE:Parkinson's disease:0.55), (ELOTUZUMAB:Parkinson's disease:0.44), (ENALAPRIL MALEATELERCANIDIPINE HYDROCHLORIDE:Parkinson's disease:0.03), (EPALRESTAT:Parkinson's disease:0.04), (EPINASTINE HYDROCHLORIDE:Parkinson's disease:1.21), (ERGOLOID MESYLATES:Parkinson's disease:0.11), (ESTRIOL:Parkinson's disease:0.88), (ETHACRYNIC ACID:Parkinson's disease:0.15), (ETHINYL ESTRADIOLNORGESTREL:Parkinson's disease:0.12), (ETRETINATE:Parkinson's disease:0.99), (FENOFIBRIC ACID:Parkinson's disease:1.27), (FENOTEROL HYDROBROMIDE:Parkinson's disease:1.08), (FERROUS BISGLYCINATE:Parkinson's disease:0.29), (FERROUS SULFATE ANHYDROUS:Parkinson's disease:0.45), (FESOTERODINE:Parkinson's disease:0.09), (FINGOLIMOD HYDROCHLORIDE:Parkinson's disease:0.65), (FLUNARIZINE HYDROCHLORIDE:Parkinson's disease:0.43), (FLUPHENAZINE DECANOATE:Parkinson's disease:0.04), (FLUTICASONE PROPIONATEFORMOTEROL FUMARATE:Parkinson's disease:0.52), (FUROSEMIDESPIRONOLACTONE:Parkinson's disease:0.08), (FURSULTIAMINE:Parkinson's disease:0.43), (GENTAMICIN SULFATE:Parkinson's disease:0.15), (GIMERACILOTERACILTEGAFUR:Parkinson's disease:0.35), (GLIPIZIDEMETFORMIN HYDROCHLORIDE:Parkinson's disease:0.17), (GLYCOPYRROLATEINDACATEROL MALEATE:Parkinson's disease:0.82), (GUANFACINE:Parkinson's disease:1.08), (GUANFACINE HYDROCHLORIDE:Parkinson's disease:0.47), (HALOPERIDOL DECANOATE:Parkinson's disease:0.4), (HYDROCORTISONE ACETATE:Parkinson's disease:0.45), (HYDROXYCHLOROQUINE DIPHOSPHATE:Parkinson's disease:0.82), (IGURATIMOD:Parkinson's disease:0.04), (ILOPROST:Parkinson's disease:0.73), (IMIQUIMOD:Parkinson's disease:0.1), (INFLIXIMAB-DYYB:Parkinson's disease:0.42), (INSULIN ASPARTINSULIN DEGLUDEC:Parkinson's disease:0.29), (INSULIN PORKINSULIN PURIFIED PORK:Parkinson's disease:0.17), (INTERFERON:Parkinson's disease:0.95), (INTERFERON ALFA-2A:Parkinson's disease:1.15), (IPRAGLIFLOZIN L-PROLINE:Parkinson's disease:0.13), (IRINOTECAN HYDROCHLORIDE:Parkinson's disease:0.91), (IRON SUCROSE:Parkinson's disease:1.17), (IRSOGLADINE MALEATE:Parkinson's disease:0.02), (IXAZOMIB:Parkinson's disease:0.54), (KETAMINE:Parkinson's disease:0.13), (LANREOTIDE:Parkinson's disease:0.44), (LANREOTIDE ACETATE:Parkinson's disease:0.22), (LAPATINIB DITOSYLATE:Parkinson's disease:0.39), (LECITHIN:Parkinson's disease:0.26), (LEUCOVORIN CALCIUM:Parkinson's disease:0.7), (LEVOCABASTINE HYDROCHLORIDE:Parkinson's disease:0.41), (LEVOLEUCOVORIN CALCIUM:Parkinson's disease:0.28), (LEVOMEPROMAZINE MALEATE:Parkinson's disease:0.08), (LEVOMILNACIPRAN HYDROCHLORIDE:Parkinson's disease:0.28), (LINSIDOMINE:Parkinson's disease:0.25), (LUTETIUM LU-177:Parkinson's disease:0.22), (MECLIZINEMECLIZINE HYDROCHLORIDE:Parkinson's disease:1.41), (METFORMIN HYDROCHLORIDESAXAGLIPTIN:Parkinson's disease:0.13), (METFORMIN PAMOATE:Parkinson's disease:0.48), (METHENAMINE MANDELATE:Parkinson's disease:0.41), (METHENOLONE ENANTHATE:Parkinson's disease:0.62), (METHYLPREDNISOLONE ACETATE:Parkinson's disease:2.22), (METRELEPTIN:Parkinson's disease:0.57), (MEXILETINE HYDROCHLORIDE:Parkinson's disease:0.25), (MICAFUNGIN:Parkinson's disease:0.19), (MIGLUSTAT:Parkinson's disease:0.19), (MILK THISTLE:Parkinson's disease:0.58), (MITIGLINIDE:Parkinson's disease:0.41), (MIZORIBINE:Parkinson's disease:0.43), (MOEXIPRIL HYDROCHLORIDE:Parkinson's disease:0.54), (MONTMORILLONITE:Parkinson's disease:0.16), (MORPHINE SULFATE\NALTREXONE HYDROCHLORIDE:Parkinson's disease:0.95), (NALOXONE:Parkinson's disease:0.89), (NALOXONE HYDROCHLORIDE:Parkinson's disease:0.07), (NARATRIPTAN HYDROCHLORIDE:Parkinson's disease:0.24), (NEFOPAM HYDROCHLORIDE:Parkinson's disease:0.23), (NIMESULIDE:Parkinson's disease:0.12), (NOREPINEPHRINE:Parkinson's disease:0.03), (NOREPINEPHRINE BITARTRATE:Parkinson's disease:0.33), (OLANZAPINE PAMOATE:Parkinson's disease:0.54), (OLAPARIB:Parkinson's disease:0.13), (OLMESARTAN:Parkinson's disease:0.54), (OLODATEROL HYDROCHLORIDETIOTROPIUM BROMIDE MONOHYDRATE:Parkinson's disease:1.58), (OSPEMIFENE:Parkinson's disease:0.08), (OXAPROZIN:Parkinson's disease:0.48), (OXYBUTYNINOXYBUTYNIN CHLORIDE:Parkinson's disease:0.94), (PANITUMUMAB:Parkinson's disease:0.39), (PASIREOTIDE:Parkinson's disease:0.05), (PATIROMER:Parkinson's disease:0.85), (PEGZEREPOETIN ALFA:Parkinson's disease:0.07), (PEMETREXED:Parkinson's disease:0.71), (PENICILLIN G:Parkinson's disease:0.42), (PENICILLIN V:Parkinson's disease:0.6), (PENTAERYTHRITOL TETRANITRATE:Parkinson's disease:0.14), (PENTAMIDINE ISETHIONATE:Parkinson's disease:0.01), (PERPHENAZINE:Parkinson's disease:0.61), (PHENOTHIAZINE:Parkinson's disease:0.11), (PHLOROGLUCINOL:Parkinson's disease:0.06), (PHLOROGLUCINOL1,3,5-TRIMETHOXYBENZENE:Parkinson's disease:0.35), (PIMECROLIMUS:Parkinson's disease:0.28), (PIMOBENDAN:Parkinson's disease:0.43), (PIPERACILLIN:Parkinson's disease:0.24), (PIROXICAM:Parkinson's disease:1.15), (PONATINIB:Parkinson's disease:1.51), (POSACONAZOLE:Parkinson's disease:0.51), (POTASSIUM CITRATETRISODIUM CITRATE DIHYDRATE:Parkinson's disease:0.26), (POVIDONE:Parkinson's disease:0.72), (PREDNISOLONE SODIUM PHOSPHATE:Parkinson's disease:0.28), (PROMAZINE:Parkinson's disease:0.21), (PROTRIPTYLINE HYDROCHLORIDE:Parkinson's disease:0.61), (QUINAGOLIDE HYDROCHLORIDE:Parkinson's disease:0.01), (RADIATION THERAPY:Parkinson's disease:0.59), (RAMUCIRUMAB:Parkinson's disease:0.58), (RETINOL:Parkinson's disease:0.89), (RILMENIDINE:Parkinson's disease:0.06), (RILPIVIRINE HYDROCHLORIDE:Parkinson's disease:0.21), (SARPOGRELATE HYDROCHLORIDE:Parkinson's disease:0.44), (SELEXIPAG:Parkinson's disease:0.44), (SIBUTRAMINE HYDROCHLORIDE:Parkinson's disease:0.23), (SIPULEUCEL-T:Parkinson's disease:0.25), (ST. JOHN'S WORT:Parkinson's disease:0.03), (SUMATRIPTANSUMATRIPTAN SUCCINATE:Parkinson's disease:1.37), (SUPLATAST TOSILATE:Parkinson's disease:0.33), (TAMOXIFEN CITRATE:Parkinson's disease:0.14), (TASIMELTEON:Parkinson's disease:0.11), (TEDUGLUTIDE:Parkinson's disease:0.81), (TELOTRISTAT ETHYL:Parkinson's disease:0.13), (TEMSIROLIMUS:Parkinson's disease:0.1), (TERIFLUNOMIDE:Parkinson's disease:0.28), (TETRACYCLINE:Parkinson's disease:0.84), (THEOPHYLLINE:Parkinson's disease:0), (TIANEPTINE:Parkinson's disease:0.1), (TIMOLOLTRAVOPROST:Parkinson's disease:0.92), (TINZAPARIN:Parkinson's disease:0.76), (TIPIRACIL HYDROCHLORIDETRIFLURIDINE:Parkinson's disease:0.23), (TRIFLUOPERAZINETRIFLUOPERAZINE HYDROCHLORIDE:Parkinson's disease:0.11), (TRIMETAZIDINE:Parkinson's disease:0.05), (TRIPTORELIN PAMOATE:Parkinson's disease:0.42), (VALDECOXIB:Parkinson's disease:0.23), (VARENICLINE TARTRATE:Parkinson's disease:2.36), (VINCRISTINE SULFATE:Parkinson's disease:0.14), (VINORELBINE TARTRATE:Parkinson's disease:0.2), (YEAST:Parkinson's disease:0.39), (ZILEUTON:Parkinson's disease:0.57), (ZUCLOPENTHIXOL DECANOATE:Parkinson's disease:0.21), (ACARBOSE:Parkinson's disease psychosis:0.01), (ACETAMINOPHENOXYCODONE HYDROCHLORIDEOXYCODONE TEREPHTHALATE:Parkinson's disease psychosis:0.15), (ACETAMINOPHENOXYCODONEOXYCODONE HYDROCHLORIDE:Parkinson's disease psychosis:0.25), (ACLIDINIUM BROMIDE:Parkinson's disease psychosis:0.12), (AMISULPRIDE:Parkinson's disease psychosis:0.11), (AMITRIPTYLINE HYDROCHLORIDE:Parkinson's disease psychosis:0.29), (ATROPINE SULFATEDIPHENOXYLATE HYDROCHLORIDE:Parkinson's disease psychosis:0.1), (BENAZEPRILBENAZEPRIL HYDROCHLORIDE:Parkinson's disease psychosis:0.06), (BENSERAZIDELEVODOPA:Parkinson's disease psychosis:0.75), (CANDESARTAN:Parkinson's disease psychosis:0.53), (CANDESARTAN CILEXETILHYDROCHLOROTHIAZIDE:Parkinson's disease psychosis:0.09), (CARVEDILOL PHOSPHATE:Parkinson's disease psychosis:1.14), (CERTOLIZUMAB PEGOL:Parkinson's disease psychosis:0.25), (CHLORTHALIDONE:Parkinson's disease psychosis:0.43), (CHOLESTYRAMINE:Parkinson's disease psychosis:0.5), (CLINDAMYCINCLINDAMYCIN PHOSPHATE:Parkinson's disease psychosis:0.66), (CLONIDINE HYDROCHLORIDE:Parkinson's disease psychosis:0.12), (CLOTRIMAZOLE:Parkinson's disease psychosis:0.09), (CORTISONEHYDROCORTISONE:Parkinson's disease psychosis:0.16), (DACLIZUMAB:Parkinson's disease psychosis:0), (DAPAGLIFLOZIN PROPANEDIOL:Parkinson's disease psychosis:0.17), (DASATINIB:Parkinson's disease psychosis:0.03), (DESLORATADINE:Parkinson's disease psychosis:0.04), (DEVICE:Parkinson's disease psychosis:0.13), (DEXTROAMPHETAMINE SULFATE:Parkinson's disease psychosis:0.06), (DOFETILIDE:Parkinson's disease psychosis:0.09), (EDOXABAN TOSYLATE:Parkinson's disease psychosis:0.01), (ELETRIPTAN HYDROBROMIDE:Parkinson's disease psychosis:0.62), (EPINEPHRINE:Parkinson's disease psychosis:0.43), (EVOLOCUMAB:Parkinson's disease psychosis:0.69), (FERROUS SULFATEFERROUS SULFATE, DRIED:Parkinson's disease psychosis:0.81), (FLUOXETINEFLUOXETINE HYDROCHLORIDE:Parkinson's disease psychosis:0.55), (HEPARIN SODIUM:Parkinson's disease psychosis:0.29), (HYALURONATE SODIUM:Parkinson's disease psychosis:0.06), (HYDROCHLOROTHIAZIDEIRBESARTAN:Parkinson's disease psychosis:0.16), (HYDROCHLOROTHIAZIDEVALSARTAN:Parkinson's disease psychosis:0.54), (HYDROMORPHONE:Parkinson's disease psychosis:0.18), (HYOSCYAMINE:Parkinson's disease psychosis:0.64), (IMIPRAMINE HYDROCHLORIDE:Parkinson's disease psychosis:0.22), (ISTRADEFYLLINE:Parkinson's disease psychosis:0.23), (LIDOCAINE HYDROCHLORIDE:Parkinson's disease psychosis:0.33), (LISDEXAMFETAMINE DIMESYLATE:Parkinson's disease psychosis:0.9), (LOMITAPIDE MESYLATE:Parkinson's disease psychosis:0.1), (METHYLPREDNISOLONE ACETATE:Parkinson's disease psychosis:0.16), (METHYLPREDNISOLONE SODIUM SUCCINATE:Parkinson's disease psychosis:0.24), (MOMETASONE FUROATE:Parkinson's disease psychosis:0.2), (NALOXONE HYDROCHLORIDEOXYCODONE HYDROCHLORIDE:Parkinson's disease psychosis:0.04), (OXYGEN:Parkinson's disease psychosis:0.59), (PANTOPRAZOLE MAGNESIUM:Parkinson's disease psychosis:0.31), (PAROXETINEPAROXETINE HYDROCHLORIDE:Parkinson's disease psychosis:0.74), (PHENOBARBITAL:Parkinson's disease psychosis:0.22), (PRAVASTATINPRAVASTATIN SODIUM:Parkinson's disease psychosis:0.33), (PREDNISOLONE ACETATE:Parkinson's disease psychosis:0.51), (PROMETHAZINE HYDROCHLORIDE:Parkinson's disease psychosis:0.4), (RABEPRAZOLE:Parkinson's disease psychosis:0.29), (RIZATRIPTAN BENZOATE:Parkinson's disease psychosis:0.27), (SACUBITRILVALSARTAN:Parkinson's disease psychosis:0.14), (SAFINAMIDE:Parkinson's disease psychosis:0.19), (SODIUM OXYBATE:Parkinson's disease psychosis:1.49), (SUMATRIPTANSUMATRIPTAN SUCCINATE:Parkinson's disease psychosis:0.55), (TERIFLUNOMIDE:Parkinson's disease psychosis:0.45), (VARENICLINE TARTRATE:Parkinson's disease psychosis:1.59), (ZINC:Parkinson's disease psychosis:0.44), (ALCOHOL:Parkinsonism:0.29), (AMOXAPINE:Parkinsonism:0.06), (BUPREN ORPHINE HYDROCHLORIDENALOXONE HYDROCHLORIDE:Parkinsonism:0.22), (CANDESARTAN CILEXETILHYDROCHLOROTHIAZIDE:Parkinsonism:0.42), (CERTOLIZUMAB PEGOL:Parkinsonism:0.45), (CLOBAZAM:Parkinsonism:0.11), (CYCLOBENZAPRINE:Parkin sonism:0.18), (DASATINIB:Parkinsonism:0.23), (DESLORATADINE:Parkinsonism:0.23), (DEX LANSOPRAZOLE:Parkinsonism:0.57), (DULOXETINE:Parkinsonism:0.05), (ENOXAPARIN:P arkinsonism:0.96), (EVEROLIMUS:Parkinsonism:0.32), (EVOLOCUMAB:Parkinsonism:0.55), (EZETIMIBE:Parkinsonism:0.18), (FLECAINIDE:Parkinsonism:0.34), (GUANFACINE:Parkinso nism:0.07), (HYDROMORPHONE HYDROCHLORIDE:Parkinsonism:0.1), (IBRUTINIB:Parkinsonism:0.56), (IMATINIB MESYLATE:Parkinsonism:0), (INDAPAMIDEPERINDOPRIL:Parkinsonism:0.29), (ISOSORB IDE:Parkinsonism:0.04), (LABETALOLLABETALOL HYDROCHLORIDE:Parkinsonism:0.05), (LEDIPASVIRSOFOSBUVIR:Parkinsonism:0.37), (L ISDEXAMFETAMINE DIMESYLATE:Parkinsonism:0.56), (LURASIDONE HYDROCHLORIDE:Parkinsonism:0.01), (METAXALONE:Parkinsonism:0.02), (METHYLPRE DNISOLONE SODIUM SUCCINATE:Parkinsonism:0.49), (METRONIDAZOLE:Parkinsonism:0.14), (MOXIFLOXACI N HYDROCHLORIDE:Parkinsonism:0.04), (NALOXONE HYDROCHLORIDEOXYCODONE HYDROCHLORIDE:Parkinsonism:0.11), (NAPROXEN:Parkinsonism:1.03), (NILOTINIB:Parki nsonism:0.14), (OXCARBAZEPINE:Parkinsonism:1.55), (OXYCODONE:Parkinsonism:0.33), (OXYCODONE HYDROCHLORIDE:Parkinsonism:0.12), (PAZOPANIB HYDROCHLORIDE:Parkinsonism:0.02), (PERGOLIDE:Parkinsonism:0.35), (PHENOBARBIT AL:Parkinsonism:0.44), (PREDNISOLONEPREDNISOLONE ACETATEPREDNISOLONE SODIUM SUCCINATE:Parkinsonism:0.14), (RANOLAZINE:Parkinsonism:0.57), (SACUBITRILVALSA RTAN:Parkinsonism:0.35), (SAFINAMIDE:Parkinsonism:0.44), (SOTALOL:Parkinsonism:0.26), (TELMISARTAN:Parkinsonism:0.54), (TERIFLUNOMIDE:Parkinsonism:0.18), (TIMOLOL:Pa rkinsonism:0.04), (TIOTROPIUM BROMIDE MONOHYDRATE:Parkinsonism:0.39), (TIZANIDINE HYDROCHLORIDE:Parkinsonism:0.42), (VERAPAMIL HYDROCHLORIDE:Parkinsonism:0.88), (IRINOTECAN:Paronychia:0.27), (LEUCOVORIN CALCIUM:Paronychia:0.05), (ACARBOSE:Paroxysmal nocturnal haemoglobinuria:0.18), (ACETAMINOPHENBUTALBITALCAFFEINE:Paroxysmal nocturnal haemoglobinuria:0.79), (ALBUMIN HUMAN:Paroxysmal nocturnal haemoglobinuria:0.24), (AMPHOTERICIN B:Paroxysmal nocturnal haemoglobinuria:0.74), (APREPITANT:Paroxysmal nocturnal haemoglobinuria:0.02), (ARMODAFINIL:Paroxysmal nocturnal haemoglobinuria:0.66), (BISACODYL OR DOCUSATE SODIUM:Paroxysmal nocturnal haemoglobinuria:0.86), (BORTEZOMIB:Paroxysmal nocturnal haemoglobinuria:0.19), (BUDESONIDEFORMOTEROL FUMARATE:Paroxysmal nocturnal haemoglobinuria:0.05), (BUPRENORPHINE HYDROCHLORIDENALOXONE HYDROCHLORIDE:Paroxysmal nocturnal haemoglobinuria:0.1), (CANAKINUMAB:Paroxysmal nocturnal haemoglobinuria:0.39), (CARFILZOMIB:Paroxysmal nocturnal haemoglobinuria:0.13), (CARVEDILOL PHOSPHATE:Paroxysmal nocturnal haemoglobinuria:0.48), (CEFUROXIME:Paroxysmal nocturnal haemoglobinuria:0.07), (CERTOLIZUMAB PEGOL:Paroxysmal nocturnal haemoglobinuria:1.03), (CETUXIMAB:Paroxysmal nocturnal haemoglobinuria:0.09), (CHOLESTYRAMINE:Paroxysmal nocturnal haemoglobinuria:1.02), (CILASTATIN SODIUMIMIPENEM:Paroxysmal nocturnal haemoglobinuria:0.31), (CILOSTAZOL:Paroxysmal nocturnal haemoglobinuria:0.33), (CIMETIDINE:Paroxysmal nocturnal haemoglobinuria:0.06), (CLOZAPINE:Paroxysmal nocturnal haemoglobinuria:0.75), (DACLIZUMAB:Paroxysmal nocturnal haemoglobinuria:0.12), (DASATINIB:Paroxysmal nocturnal haemoglobinuria:0.67), (DIGITOXIN:Paroxysmal nocturnal haemoglobinuria:0.02), (EVOLOCUMAB:Paroxysmal nocturnal haemoglobinuria:0.41), (FORMOTEROL FUMARATE DIHYDRATE\MOMETASONE FUROATE:Paroxysmal nocturnal haemoglobinuria:0.02), (HYDROCHLOROTHIAZIDEIRBESARTAN:Paroxysmal nocturnal haemoglobinuria:0.36), (HYDROCHLOROTHIAZIDETELMISARTAN:Paroxysmal nocturnal haemoglobinuria:0), (IBRUTINIB:Paroxysmal nocturnal haemoglobinuria:1.22), (ILOPROST:Paroxysmal nocturnal haemoglobinuria:0.1), (IMATINIB MESYLATE:Paroxysmal nocturnal haemoglobinuria:0.8), (INDAPAMIDE:Paroxysmal nocturnal haemoglobinuria:0.31), (INDAPAMIDEPERINDOPRIL:Paroxysmal nocturnal haemoglobinuria:0.23), (INVESTIGATIONAL PRODUCT:Paroxysmal nocturnal haemoglobinuria:0.12), (IRINOTECAN HYDROCHLORIDE:Paroxysmal nocturnal haemoglobinuria:0.03), (IRON SUCROSE:Paroxysmal nocturnal haemoglobinuria:0.33), (LABETALOL HYDROCHLORIDE:Paroxysmal nocturnal haemoglobinuria:0.09), (LERCANIDIPINE:Paroxysmal nocturnal haemoglobinuria:0.35), (LERCANIDIPINE HYDROCHLORIDE:Paroxysmal nocturnal haemoglobinuria:0.75), (LEVALBUTEROL HYDROCHLORIDE:Paroxysmal nocturnal haemoglobinuria:0.58), (LEVOLEUCOVORIN CALCIUM:Paroxysmal nocturnal haemoglobinuria:0.14), (LISDEXAMFETAMINE DIMESYLATE:Paroxysmal nocturnal haemoglobinuria:0.6), (METAXALONE:Paroxysmal nocturnal haemoglobinuria:0.58), (METHOCARBAMOL:Paroxysmal nocturnal haemoglobinuria:0.28), (METHYLPREDNISOLONE ACETATE:Paroxysmal nocturnal haemoglobinuria:0.51), (MICAFUNGIN:Paroxysmal nocturnal haemoglobinuria:0.1), (MINOCYCLINEMINOCYCLINE HYDROCHLORIDE:Paroxysmal nocturnal haemoglobinuria:0.34), (MODAFINIL:Paroxysmal nocturnal haemoglobinuria:0.1), (NABUMETONE:Paroxysmal nocturnal haemoglobinuria:0.06), (NIACIN:Paroxysmal nocturnal haemoglobinuria:0.49), (NICOTINE:Paroxysmal nocturnal haemoglobinuria:0.19), (OXCARBAZEPINE:Paroxysmal nocturnal haemoglobinuria:1.55), (OXYBUTYNIN CHLORIDE:Paroxysmal nocturnal haemoglobinuria:0.28), (PAZOPANIB HYDROCHLORIDE:Paroxysmal nocturnal haemoglobinuria:0.13), (PONATINIB:Paroxysmal nocturnal haemoglobinuria:0.51), (PRIMIDONE:Paroxysmal nocturnal haemoglobinuria:0.76), (PROPAFENONE HYDROCHLORIDE:Paroxysmal nocturnal haemoglobinuria:0.05), (ROFLUMILAST:Paroxysmal nocturnal haemoglobinuria:0.46), (SACUBITRILVALSARTAN:Paroxysmal nocturnal haemoglobinuria:0.17), (SECUKINUMAB:Paroxysmal nocturnal haemoglobinuria:0.19), (SODIUM OXYBATE:Paroxysmal nocturnal haemoglobinuria:1.04), (SOLIFENACIN SUCCINATE:Paroxysmal nocturnal haemoglobinuria:0.13), (TERBINAFINE HYDROCHLORIDE:Paroxysmal nocturnal haemoglobinuria:0.1), (TERBUTALINE SULFATE:Paroxysmal nocturnal haemoglobinuria:0.24), (TIOTROPIUM:Paroxysmal nocturnal haemoglobinuria:0.08), (TIZANIDINE:Paroxysmal nocturnal haemoglobinuria:0.54), (TOCILIZUMAB:Paroxysmal nocturnal haemoglobinuria:0), (TOFACITINIB CITRATE:Paroxysmal nocturnal haemoglobinuria:0), (UMECLIDINIUM BROMIDEVILANTEROL TRIFENATATE:Paroxysmal nocturnal haemoglobinuria:0.21), (VARENICLINE TARTRATE:Paroxysmal nocturnal haemoglobinuria:0.75), (ZONISAMIDE:Paroxysmal nocturnal haemoglobinuria:0.03), (ANASTROZOLE:Partial seizures:0.14), (AZITHROMYCIN DIHYDRATE:Partial seizures:0.16), (BISACODYL OR DOCUSATE SODIUM:Partial seizures:0.2), (BUMETANIDE:Partial seizures:0.15), (BUPRENORPHINE HYDROCHLORIDE:Partial seizures:0.39), (BUPRENORPHINE HYDROCHLORIDENALOXONE HYDROCHLORIDE:Partial seizures:0.2), (BUPROPION:Partial seizures:0.63), (CALCITRIOL:Partial seizures:0.14), (CALCIUM CARBONATECHOLECALCIFEROL:Partial seizures:0.62), (CANDESARTAN:Partial seizures:0.22), (CANDESARTAN CILEXETIL:Partial seizures:0.62), (CANDESARTAN CILEXETILHYDROCHLOROTHIAZIDE:Partial seizures:0.1), (CHLORPROMAZINE:Partial seizures:0.16), (CHOLESTYRAMINE:Partial seizures:0.39), (DALFAMPRIDINE:Partial seizures:0.96), (DASATINIB:Partial seizures:0.03), (DICYCLOMINE HYDROCHLORIDE:Partial seizures:0.04), (DIPHENHYDRAMINE:Partial seizures:0.62), (DUTASTERIDE:Partial seizures:0.19), (EVOLOCUMAB:Partial seizures:0.32), (GLIPIZIDE:Partial seizures:0.01), (HYDROMORPHONE:Partial seizures:0.01), (HYDROMORPHONE HYDROCHLORIDE:Partial seizures:0.64), (HYDROXYZINE PAMOATE:Partial seizures:0.95), (INDAPAMIDE:Partial seizures:0.16), (INDAPAMIDEPERINDOPRIL:Partial seizures:0.08), (INDOMETHACIN:Partial seizures:0.1), (LEDIPASVIRSOFOSBUVIR:Partial seizures:0.15), (LITHIUM CARBONATE:Partial seizures:0.46), (METHADONE HYDROCHLORIDE:Partial seizures:0.09), (METHYLPREDNISOLONE ACETATE:Partial seizures:0.65), (METRONIDAZOLE:Partial seizures:1.2), (METRONIDAZOLEMETRONIDAZOLE HYDROCHLORIDE:Partial seizures:0.11), (MOXIFLOXACIN HYDROCHLORIDE:Partial seizures:0.39), (NALOXONE HYDROCHLORIDEOXYCODONE HYDROCHLORIDE:Partial seizures:0.09), (NEBIVOLOL:Partial seizures:0.75), (NILOTINIB:Partial seizures:0.11), (PENICILLIN:Partial seizures:0.09), (POLYETHYLENE GLYCOL 3350:Partial seizures:0.35), (PREDNISOLONEPREDNISOLONE ACETATEPREDNISOLONE SODIUM SUCCINATE:Partial seizures:0.03), (RIZATRIPTAN BENZOATE:Partial seizures:0.2), (SACUBITRILVALSARTAN:Partial seizures:0.31), (SECUKINUMAB:Partial seizures:0.48), (SODIUM OXYBATE:Partial seizures:0.77), (TERIFLUNOMIDE:Partial seizures:0.43), (TIOTROPIUM BROMIDE MONOHYDRATE:Partial seizures:0.61), (TOCILIZUMAB:Partial seizures:0.5), (TOFACITINIB CITRATE:Partial seizures:0.04), (TORSEMIDE:Partial seizures:0.3), (ATENOLOL:Pelvic pain:0.56), (BISACODYL OR DOCUSATE SODIUM:Pelvic pain:0.03), (BUPROPION:Pelvic pain:0.16), (CERTOLIZUMAB PEGOL:Pelvic pain:1.1), (CHOLESTYRAMINE:Pelvic pain:0.09), (DASATINIB:Pelvic pain:0.12), (DILTIAZEM HYDROCHLORIDE:Pelvic pain:1.02), (DIPHENHYDRAMINE:Pelvic pain:0.56), (DIPHENHYDRAMINE HYDROCHLORIDE:Pelvic pain:0.04), (ENALAPRIL:Pelvic pain:0.96), (EVOLOCUMAB:Pelvic pain:0.29), (HYDROCHLOROTHIAZIDELISINOPRIL:Pelvic pain:0.1), (HYDROXYZINE PAMOATE:Pelvic pain:0.01), (IBRUTINIB:Pelvic pain:0.56), (INDAPAMIDEPERINDOPRIL:Pelvic pain:0.02), (ISOSORBIDE MONONITRATE:Pelvic pain:0.73), (LISDEXAMFETAMINE DIMESYLATE:Pelvic pain:0), (METHYLPREDNISOLONE SODIUM SUCCINATE:Pelvic pain:0.84), (NEBIVOLOL:Pelvic pain:0.24), (NILOTINIB:Pelvic pain:0.04), (OXCARBAZEPINE:Pelvic pain:0.44), (PAROXETINE:Pelvic pain:0.18), (PENICILLIN:Pelvic pain:0.15), (POTASSIUM:Pelvic pain:0.21), (PRAMIPEXOLE DIHYDROCHLORIDE:Pelvic pain:0.13), (RISEDRONATE SODIUM:Pelvic pain:0.29), (SACUBITRILVALSARTAN:Pelvic pain:0.11), (SUMATRIPTANSUMATRIPTAN SUCCINATE:Pelvic pain:0.43), (TAMSULOSIN:Pelvic pain:0.76), (TOFACITINIB CITRATE:Pelvic pain:0.16), (TORSEMIDE:Pelvic pain:0.21), (VARENICLINE TARTRATE:Pelvic pain:0.34), (EVOLOCUMAB:Pemphigoid:0.26), (POTASSIUM:Pemphigoid:0.07), (SACUBITRI LVALSARTAN:Pemphigoid:0.18), (TERIFLUNOMIDE:Pemphigoid:0.21), (.ALPHA.1-PROTEINASE INHIBITOR HUMAN:Peptic ulcer:0.04), (ACENOCOUMAROL:Peptic ulcer:0.27), (ACETAMINOPHENOXYCODONEOXYCODONE HYDROCHLORIDE:Peptic ulcer:0.08), (ADO-TRASTUZUMAB EMTANSINE:Peptic ulcer:0.27), (ALCOHOL:Peptic ulcer:0.09), (ALENDRONIC ACID:Peptic ulcer:0.32), (AMIODARONE HYDROCHLORIDE:Peptic ulcer:1.2), (BENAZEPRIL HYDROCHLORIDE:Peptic ulcer:0.54), (BENAZEPRILBENAZEPRIL HYDROCHLORIDE:Peptic ulcer:0.09), (BUDESONIDEFORMOTEROL FUMARATE:Peptic ulcer:0.36), (BUMETANIDE:Peptic ulcer:1.31), (BUPROPION:Peptic ulcer:0.68), (CALCIUM ACETATE:Peptic ulcer:0.96), (CALCIUM CITRATE:Peptic ulcer:0.34), (CANDESARTAN:Peptic ulcer:1.15), (CANDESARTAN CILEXETILHYDROCHLOROTHIAZIDE:Peptic ulcer:0.84), (CARVEDILOL PHOSPHATE:Peptic ulcer:0.58), (CEFTRIAXONE SODIUM:Peptic ulcer:0.83), (CHOLESTYRAMINE:Peptic ulcer:0.71), (CLINDAMYCIN:Peptic ulcer:0.2), (DABIGATRAN:Peptic ulcer:0.19), (DACLIZUMAB:Peptic ulcer:0.05), (DESLORATADINE:Peptic ulcer:0.57), (DICLOFENAC:Peptic ulcer:1.33), (DIMENHYDRINATE:Peptic ulcer:0.22), (DOFETILIDE:Peptic ulcer:0.26), (EDOXABAN TOSYLATE:Peptic ulcer:0.51), (ELETRIPTAN HYDROBROMIDE:Peptic ulcer:0.34), (EPIRUBICIN:Peptic ulcer:0.18), (FESOTERODINE FUMARATE:Peptic ulcer:0.41), (FLUTICASONE FUROATE:Peptic ulcer:0.23), (FLUTICASONE FUROATESALMETEROL XINAFOATE:Peptic ulcer:0.56), (FORMOTEROL FUMARATE DIHYDRATE\MOMETASONE FUROATE:Peptic ulcer:0.25), (FULVESTRANT:Peptic ulcer:0.26), (HYDROCHLOROTHIAZIDEIRBESARTAN:Peptic ulcer:0.31), (HYDROMORPHONE:Peptic ulcer:0.71), (HYDROXYCHLOROQUINE DIPHOSPHATE:Peptic ulcer:0.19), (HYDROXYZINE PAMOATE:Peptic ulcer:0.84), (ILOPROST:Peptic ulcer:0.53), (IMATINIB MESYLATE:Peptic ulcer:0.68), (INDACATEROL:Peptic ulcer:0.03), (IPRATROPIUM:Peptic ulcer:0.96), (LERCANIDIPINE HYDROCHLORIDE:Peptic ulcer:0.46), (LISDEXAMFETAMINE DIMESYLATE:Peptic ulcer:0.55), (LITHIUM CARBONATE:Peptic ulcer:0.6), (METHOCARBAMOL:Peptic ulcer:0.04), (METHYLPREDNISOLONE ACETATE:Peptic ulcer:0.16), (MIDODRINE:Peptic ulcer:0.12), (MODAFINIL:Peptic ulcer:0.13), (MOXIFLOXACIN:Peptic ulcer:0.19), (NINTEDANIB:Peptic ulcer:0.37), (NITROFURANTOINNITROFURANTOIN MONOHYDRATE:Peptic ulcer:0.38), (NORTRIPTYLINE:Peptic ulcer:0.23), (OXAZEPAM:Peptic ulcer:0.04), (OXCARBAZEPINE:Peptic ulcer:1.04), (PAZOPANIB HYDROCHLORIDE:Peptic ulcer:0.54), (PHENOBARBITAL:Peptic ulcer:0.19), (PITAVASTATIN CALCIUM:Peptic ulcer:0.22), (POSACONAZOLE:Peptic ulcer:0.05), (PRAMIPEXOLE DIHYDROCHLORIDE:Peptic ulcer:0.59), (RISEDRONATE SODIUM:Peptic ulcer:0.35), (RIZATRIPTAN BENZOATE:Peptic ulcer:0.26), (SACUBITRILVALSARTAN:Peptic ulcer:0.78), (SCOPOLAMINE:Peptic ulcer:0.28), (SODIUM OXYBATE:Peptic ulcer:0.87), (SOTALOL:Peptic ulcer:0.66), (SUMATRIPTAN:Peptic ulcer:0.04), (TERBUTALINE SULFATE:Peptic ulcer:0.58), (UMECLIDINIUM BROMIDEVILANTEROL TRIFENATATE:Peptic ulcer:0.63), (VALGANCICLOVIR HYDROCHLORIDE:Peptic ulcer:0.27), (VINCRISTINE SULFATE:Peptic ulcer:0.03), (VITAMIN B:Peptic ulcer:0.64), (BUMETANIDE:Percutaneous coronary intervention:0.04), (CANDESARTAN CILEXETIL:Percutaneous coronary intervention:0.49), (CANDESARTAN CILEXETILHYDROCHLOROTHIAZIDE:Percutaneous coronary intervention:0.13), (DEVICE:Percutaneous coronary intervention:0.18), (DEXLANSOPRAZOLE:Percutaneous coronary intervention:0.26), (DICLOFENAC SODIUM:Percutaneous coronary intervention:0.06), (DULOXETINE HYDROCHLORIDE:Percutaneous coronary intervention:0.1), (EDOXABAN TOSYLATE:Percutaneous coronary intervention:0.13), (ESCITALOPRAM OXALATE:Percutaneous coronary intervention:0.17), (FERROUS SULFATE:Percutaneous coronary intervention:0.05), (FERROUS SULFATEFERROUS SULFATE, DRIED:Percutaneous coronary intervention:0.4), (FLECAINIDE:Percutaneous coronary intervention:0.3), (FLUTICASONE FUROATEVILANTEROL TRIFENATATE:Percutaneous coronary intervention:0.09), (IBRUTINIB:Percutaneous coronary intervention:0.09), (IDARUCIZUMAB:Percutaneous coronary intervention:0.16), (ILOPROST:Percutaneous coronary intervention:0.1), (LERCANIDIPINE:Percutaneous coronary intervention:0.05), (NAPROXEN:Percutaneous coronary intervention:0.29), (PRAVASTATIN:Percutaneous coronary intervention:0.54), (UMECLIDINIUM BROMIDEVILANTEROL TRIFENATATE:Percutaneous coronary intervention:0.01), (EVOLOCUMAB:Pericardial effusion:0.01), (METRONIDAZOLE:Pericardial effusion:0.12), (RIOCIGUAT:Pericardial effusion:0.61), (DARUNAVIRRITONAVIR:Perinatal HIV infection:0.08), (DESFLURANE:Perioperative analgesia:0.39), (ACETYLCYSTEINE:Peripheral arterial occlusive disease:0.04), (ACLIDINIUM BROMIDE:Peripheral arterial occlusive disease:0.04), (AGALSIDASE BETA:Peripheral arterial occlusive disease:0.34), (ALENDRONIC ACID:Peripheral arterial occlusive disease:0.66), (AMILORIDE HYDROCHLORIDEHYDROCHLOROTHIAZIDE:Peripheral arterial occlusive disease:0.2), (AMLODIPINE BESYLATEHYDROCHLOROTHIAZIDEVALSARTAN:Peripheral arterial occlusive disease:0.29), (AMPHETAMINE ASPARTATEAMPHETAMINE SULFATEDEXTROAMPHETAMINE SACCHARATEDEXTROAMPHETAMINE SULFATE:Peripheral arterial occlusive disease:0.2), (ANAGLIPTIN:Peripheral arterial occlusive disease:0.58), (APRINDINE HYDROCHLORIDE:Peripheral arterial occlusive disease:0.02), (BENIDIPINE:Peripheral arterial occlusive disease:0.32), (BEPRIDIL:Peripheral arterial occlusive disease:1.24), (BETAHISTINE MESILATE:Peripheral arterial occlusive disease:0.37), (BISACODYL OR DOCUSATE SODIUM:Peripheral arterial occlusive disease:0.89), (BUDESONIDEFORMOTEROL FUMARATE:Peripheral arterial occlusive disease:0.41), (CANAGLIFLOZINMETFORMIN HYDROCHLORIDE:Peripheral arterial occlusive disease:0.22), (CARBIMAZOLE:Peripheral arterial occlusive disease:0.36), (CARFILZOMIB:Peripheral arterial occlusive disease:0.51), (CARVEDILOL PHOSPHATE:Peripheral arterial occlusive disease:1.16), (CEFOTAXIME SODIUM:Peripheral arterial occlusive disease:0.2), (CHOLESTYRAMINE:Peripheral arterial occlusive disease:0.59), (CIFENLINE:Peripheral arterial occlusive disease:0.43), (CIPROFLOXACIN HYDROCHLORIDE:Peripheral arterial occlusive disease:0.03), (CLOZAPINE:Peripheral arterial occlusive disease:1.02), (COLESEVELAM HYDROCHLORIDE:Peripheral arterial occlusive disease:0.04), (DABIGATRAN ETEXILATE:Peripheral arterial occlusive disease:0.06), (DALTEPARIN:Peripheral arterial occlusive disease:0.08), (DANAZOL:Peripheral arterial occlusive disease:0.03), (DAPAGLIFLOZIN PROPANEDIOL:Peripheral arterial occlusive disease:0.22), (DEGARELIX:Peripheral arterial occlusive disease:0.23), (DEXAMETHASONE ACETATE:Peripheral arterial occlusive disease:0.38), (DOBUTAMINE HYDROCHLORIDE:Peripheral arterial occlusive disease:0.36), (DOCUSATE:Peripheral arterial occlusive disease:0.8), (DOCUSATE CALCIUM:Peripheral arterial occlusive disease:0), (DOFETILIDE:Peripheral arterial occlusive disease:0.77), (ECONAZOLE:Peripheral arterial occlusive disease:0.18), (ELETRIPTAN HYDROBROMIDE:Peripheral arterial occlusive disease:0.16), (EMPAGLIFLOZINLINAGLIPTIN:Peripheral arterial occlusive disease:0.09), (ENOXAPARIN:Peripheral arterial occlusive disease:2.78), (ERYTHROMYCIN:Peripheral arterial occlusive disease:0.18), (ETRETINATE:Peripheral arterial occlusive disease:0.16), (EVEROLIMUS:Peripheral arterial occlusive disease:1.57), (FESOTERODINE FUMARATE:Peripheral arterial occlusive disease:0.6), (FLUDROCORTISONE ACETATE:Peripheral arterial occlusive disease:0.13), (FLUOXETINEFLUOXETINE HYDROCHLORIDE:Peripheral arterial occlusive disease:0.34), (FLUTICASONE:Peripheral arterial occlusive disease:0.45), (FLUTICASONEFLUTICASONE PROPIONATE:Peripheral arterial occlusive disease:0.38), (FORMOTEROL FUMARATE:Peripheral arterial occlusive disease:0.4), (FURSULTIAMINE:Penpheral arterial occlusive disease:0.2), (FUSIDIC ACID:Peripheral arterial occlusive disease:0.21), (GIMERACILOTERACILTEGAFUR:Peripheral arterial occlusive disease:0.11), (GLIMEPIRIDEROSIGLITAZONE MALEATE:Peripheral arterial occlusive disease:0.27), (GUANABENZ ACETATE:Peripheral arterial occlusive disease:0.22), (HYDROXYZINE PAMOATE:Peripheral arterial occlusive disease:0.53), (ILOPROST:Peripheral arterial occlusive disease:1.36), (IMATINIB MESYLATE:Peripheral arterial occlusive disease:1.2), (INFLUENZA VIRUS VACCINE:Peripheral arterial occlusive disease:0.11), (INSULIN ASPARTINSULIN DEGLUDEC:Peripheral arterial occlusive disease:0.19), (IPRAGLIFLOZIN L-PROLINE:Peripheral arterial occlusive disease:0.13), (IRINOTECAN HYDROCHLORIDE:Peripheral arterial occlusive disease:0.21), (IVABRADINE:Peripheral arterial occlusive disease:0.16), (LABETALOLLABETALOL HYDROCHLORIDE:Peripheral arterial occlusive disease:1.37), (LACOSAMIDE:Peripheral arterial occlusive disease:0.19), (LEDIPASVIRSOFOSBUVIR:Peripheral arterial occlusive disease:1.58), (LEUCOVORIN CALCIUM:Peripheral arterial occlusive disease:0.08), (LEVOLEUCOVORIN CALCIUM:Peripheral arterial occlusive disease:0.04), (LIDOCAINEPRILOCAINE:Peripheral arterial occlusive disease:0.12), (METAXALONE:Peripheral arterial occlusive disease:0.01), (METFORMIN HYDROCHLORIDEROSIGLITAZONE MALEATE:Peripheral arterial occlusive disease:0.5), (METFORMIN HYDROCHLORIDEVILDAGLIPTIN:Peripheral arterial occlusive disease:0.47), (METHADONE HYDROCHLORIDE:Peripheral arterial occlusive disease:0.66), (METHYLPREDNISOLONE ACETATE:Peripheral arterial occlusive disease:0.36), (MIDODRINE:Peripheral arterial occlusive disease:0.62), (MINOCYCLINEMINOCYCLINE HYDROCHLORIDE:Peripheral arterial occlusive disease:0.03), (NADOLOL:Peripheral arterial occlusive disease:0.23), (NADROPARIN CALCIUM:Peripheral arterial occlusive disease:0.96), (NOREPINEPHRINE BITARTRATE:Peripheral arterial occlusive disease:0.12), (NORTRIPTYLINE:Peripheral arterial occlusive disease:0.14), (OLODATEROL HYDROCHLORIDETIOTROPIUM BROMIDE MONOHYDRATE:Peripheral arterial occlusive disease:0.42), (OMEPRAZOLE SODIUM:Peripheral arterial occlusive disease:0.67), (PANTOPRAZOLE MAGNESIUM:Peripheral arterial occlusive disease:0.8), (PAROXETINEPAROXETINE HYDROCHLORIDE:Peripheral arterial occlusive disease:0.69), (PATIROMER:Peripheral arterial occlusive disease:0.22), (PEMETREXED DISODIUM:Peripheral arterial occlusive disease:0.32), (PENICILLIN:Peripheral arterial occlusive disease:0.07), (PHENOBARBITAL:Peripheral arterial occlusive disease:0.93), (PIMOBENDAN:Peripheral arterial occlusive disease:1.38), (POLYSTYRENE:Peripheral arterial occlusive disease:0.43), (POTASSIUM CITRATETRISODIUM CITRATE DIHYDRATE:Peripheral arterial occlusive disease:0.05), (PROBIOTICS NOS:Peripheral arterial occlusive disease:0.47), (PROBUCOL:Peripheral arterial occlusive disease:0.22), (PROMETHAZINEPROMETHAZINE HYDROCHLORIDE:Peripheral arterial occlusive disease:0.6), (RIOCIGUAT:Peripheral arterial occlusive disease:0.13), (SCOPOLAMINE:Peripheral arterial occlusive disease:0.4), (SODIUM PHOSPHATE, DIBASIC\SODIUM PHOSPHATE, MONOBASIC:Peripheral arterial occlusive disease:0.25), (SULFAMETHOXAZOLE:Peripheral arterial occlusive disease:0.32), (TAFAMIDIS MEGLUMINE:Peripheral arterial occlusive disease:0.08), (TERBUTALINE:Peripheral arterial occlusive disease:0.19), (THIOCTIC ACID:Peripheral arterial occlusive disease:0.22), (TROSPIUM CHLORIDE:Peripheral arterial occlusive disease:0.22), (UMECLIDINIUM BROMIDEVILANTEROL TRIFENATATE:Peripheral arterial occlusive disease:0.63), (VALGANCICLOVIR HYDROCHLORIDE:Peripheral arterial occlusive disease:0.41), (VITAMIN B:Peripheral arterial occlusive disease:0.47), (ZOLEDRONIC ACID:Peripheral arterial occlusive disease:0.43), (ZONISAMIDE:Peripheral arterial occlusive disease:0.12), (AMIODARONE:Peripheral sensory neuropathy:0.35), (AMIODARONE HYDROCHLORIDE:Peripheral sensory neuropathy:0.05), (ANASTROZOLE:Peripheral sensory neuropathy:1.06), (BISACODYL OR DOCUSATE SODIUM:Peripheral sensory neuropathy:0.04), (BUMETANIDE:Peripheral sensory neuropathy:0.72), (CANDESARTAN CILEXETIL:Peripheral sensory neuropathy:0.16), (CERTOLIZUMAB PEGOL:Peripheral sensory neuropathy:0.69), (CHOLESTYRAMINE:Peripheral sensory neuropathy:0.24), (CLINDAMYCINCLINDAMYCIN PHOSPHATE:Peripheral sensory neuropathy:0.54), (CLONIDINE:Peripheral sensory neuropathy:0.17), (DASATINIB:Peripheral sensory neuropathy:0.33), (DEXLANSOPRAZOLE:Peripheral sensory neuropathy:0.67), (DILTIAZEM:Peripheral sensory neuropathy:0.37), (ERGOCALCIFEROL:Peripheral sensory neuropathy:0.23), (ESZOPICLONE:Peripheral sensory neuropathy:0.05), (FERROUS SULFATEFERROUS SULFATE, DRIED:Peripheral sensory neuropathy:0.45), (HYDROCHLOROTHIAZIDELISINOPRIL:Peripheral sensory neuropathy:0.18), (IRINOTECAN:Peripheral sensory neuropathy:0.74), (LEVOLEUCOVORIN CALCIUM:Peripheral sensory neuropathy:0.2), (METHYLPREDNISOLONE SODIUM SUCCINATE:Peripheral sensory neuropathy:0.56), (METOPROLOL TARTRATE:Peripheral sensory neuropathy:0.23), (NEBIVOLOL:Peripheral sensory neuropathy:0.08), (NILOTINIB:Peripheral sensory neuropathy:0.78), (NITROGLYCERIN:Peripheral sensory neuropathy:0.17), (PAZOPANIB HYDROCHLORIDE:Peripheral sensory neuropathy:0.06), (SACUBITRILVALSARTAN:Peripheral sensory neuropathy:0.2), (TOPIRAMATE:Peripheral sensory neuropathy:0.2), (WARFARIN SODIUM:Peripheral sensory neuropathy:0.47), (.ALPHA.1-PROTEINASE INHIBITOR HUMAN:Peripheral swelling:0.59), (ADO-TRASTUZUMAB EMTANSINE:Peripheral swelling:0.36), (AGALSIDASE BETA:Peripheral swelling:0.33), (ALCOHOL:Penpheral swelling:0.26), (ALISKIREN HEMIFUMARATE:Peripheral swelling:0.37), (AMANTADINE:Peripheral swelling:0.33), (AMLODIPINEVALSARTAN:Peripheral swelling:0.29), (ANTIHISTAMINES NOS:Peripheral swelling:0.18), (BUPRENORPHINE HYDROCHLORIDE:Peripheral swelling:0.35), (CALCIUM\MAGNESIUM\ZINC:Peripheral swelling:0.29), (CANAKINUMAB:Peripheral swelling:0.61), (CHLORDIAZEPOXIDE HYDROCHLORIDECLIDINIUM BROMIDE:Peripheral swelling:0.25), (CHOLESTYRAMINE:Peripheral swelling:1.29), (CILASTATIN SODIUMIMIPENEM:Peripheral swelling:0.34), (CIMETIDINE:Penpheral swelling:1.07), (CINACALCET HYDROCHLORIDE:Peripheral swelling:0.73), (CLOZAPINE:Peripheral swelling:0.68), (CODEINE PHOSPHATEGUAIFENESIN:Peripheral swelling:0.17), (DABIGATRAN:Peripheral swelling:0.02), (DACLIZUMAB:Peripheral swelling:0.87), (DASATINIB:Peripheral swelling:1.37), (DICLOFENAC SODIUMMISOPROSTOL:Peripheral swelling:0.12), (DIMENHYDRINATE:Peripheral swelling:0.63), (DIPYRIDAMOLE:Peripheral swelling:0.06), (DORZOLAMIDE HYDROCHLORIDE:Peripheral swelling:0.16), (DOXEPIN HYDROCHLORIDE:Peripheral swelling:0.44), (EMPAGLIFLOZINLINAGLIPTIN:Peripheral swelling:0.9), (ETORICOXIB:Peripheral swelling:0.3), (FINGOLIMOD HYDROCHLORIDE:Peripheral swelling:0.66), (FLUDROCORTISONE ACETATE:Peripheral swelling:0.18), (FLUTICASONE FUROATE:Peripheral swelling:0.75), (FORMOTEROL FUMARATE\MOMETASONE FUROATE:Peripheral swelling:0.67), (GLYCOPYRRONIUM:Peripheral swelling:0.11), (GLYCOPYRRONIUMINDACATEROL:Peripheral swelling:0.59), (GUANFACINE:Peripheral swelling:0.11), (HYDROCHLOROTHIAZIDEIRBESARTAN:Peripheral swelling:0.74), (HYDROCHLOROTHIAZIDETELMISARTAN:Peripheral swelling:0.27), (HYDROCODONE BITARTRATEIBUPROFEN:Peripheral swelling:0.02), (INDAPAMIDEPERINDOPRIL:Peripheral swelling:0.59), (INSULIN DEGLUDEC:Peripheral swelling:0.44), (INSULIN GLULISINE:Peripheral swelling:0.21), (ISOSORBIDE DINITRATE:Peripheral swelling:0.09), (LACTASE:Peripheral swelling:0.19), (LAPATINIB DITOSYLATE:Peripheral swelling:0.06), (LEDIPASVIRSOFOSBUVIR:Peripheral swelling:0.48), (LERCANIDIPINE HYDROCHLORIDE:Peripheral swelling:0.14), (LEVOMEFOLATE CALCIUM:Peripheral swelling:0.15), (LITHIUM CARBONATE:Peripheral swelling:0.85), (LOMITAPIDE MESYLATE:Peripheral swelling:0.57), (MEPOLIZUMAB:Peripheral swelling:0.38), (METFORMIN HYDROCHLORIDEVILDAGLIPTIN:Peripheral swelling:0.02), (MIDODRINE:Peripheral swelling:0.61), (MINOCYCLINE HYDROCHLORIDE:Peripheral swelling:0.15), (MINOCYCLINEMINOCYCLINE HYDROCHLORIDE:Peripheral swelling:0.97), (NALOXONE HYDROCHLORIDEOXYCODONE HYDROCHLORIDE:Peripheral swelling:0.73), (NITROFURANTOINNITROFURANTOIN MONOHYDRATE:Peripheral swelling:0.4), (NORTRIPTYLINE HYDROCHLORIDE:Peripheral swelling:0.33), (PANTOPRAZOLE MAGNESIUM:Peripheral swelling:1.69), (PARATHYROID HORMONE:Peripheral swelling:0.19), (PAZOPANIB HYDROCHLORIDE:Peripheral swelling:0.7), (PEGINTERFERON BETA-1A:Peripheral swelling:0.67), (PEMETREXED DISODIUM:Peripheral swelling:0.7), (PENICILLIN:Peripheral swelling:0.79), (PONATINIB:Peripheral swelling:1.29), (POSACONAZOLE:Peripheral swelling:0.41), (PRAZOSIN HYDROCHLORIDE:Peripheral swelling:0.19), (PREDNISOLONE SODIUM PHOSPHATE:Peripheral swelling:0.01), (PREDNISOLONEPREDNISOLONE ACETATEPREDNISOLONE SODIUM SUCCINATE:Peripheral swelling:0.14), (PSYLLIUM HUSK:Peripheral swelling:0.39), (RABEPRAZOLE:Peripheral swelling:1.1), (RIVASTIGMINE TARTRATE:Peripheral swelling:0.48), (SACCHAROMYCES CEREVISIAE:Peripheral swelling:0.13), (SELEXIPAG:Peripheral swelling:0.5), (TERAZOSIN HYDROCHLORIDE:Peripheral swelling:0.25), (TETRACYCLINE:Peripheral swelling:0.58), (TOBRAMYCIN:Peripheral swelling:0.29), (VANCOMYCIN HYDROCHLORIDE:Peripheral swelling:0.33), (ZILEUTON:Peripheral swelling:0.1), (ZONISAMIDE:Peripheral swelling:0.14), (IBRITUMOMAB TIUXETAN:Peripheral T-cell lymphoma unspecified:0.21), (IDELALISIB:Peripheral T-cell lymphoma unspecified:0.08), (METRONIDAZOLE:Peripheral T-cell lymphoma unspecified:0.54), (ACENOCOUMAROL:Peripheral vascular disorder:0.03), (ACETAMINOPHENBUTALBITALCAFFEINE:Peripheral vascular disorder:0.17), (ALENDRONIC ACID:Peripheral vascular disorder:0.08), (ATROPINE SULFATEDIPHENOXYLATE HYDROCHLORIDE:Peripheral vascular disorder:0.19), (BENAZEPRILBENAZEPRIL HYDROCHLORIDE:Peripheral vascular disorder:0.09), (BISACODYL OR DOCUSATE SODIUM:Peripheral vascular disorder:0.67), (CANAGLIFLOZINMETFORMIN HYDROCHLORIDE:Peripheral vascular disorder:0.1), (CANDESARTAN CILEXETILHYDROCHLOROTHIAZIDE:Peripheral vascular disorder:0.71), (CARFILZOMIB:Peripheral vascular disorder:0.14), (CARVEDILOL PHOSPHATE:Peripheral vascular disorder:0.36), (CERTOLIZUMAB PEGOL:Peripheral vascular disorder:1.43), (CHOLESTYRAMINE:Peripheral vascular disorder:0.62), (CINACALCET HYDROCHLORIDE:Peripheral vascular disorder:0.04), (CLINDAMYCINCLINDAMYCIN PHOSPHATE:Peripheral vascular disorder:1.08), (CORTISONEHYDROCORTISONE:Peripheral vascular disorder:0.69), (DAPAGLIFLOZIN PROPANEDIOL:Peripheral vascular disorder:0.25), (DASATINIB:Peripheral vascular disorder:0.93), (DESLORATADINE:Peripheral vascular disorder:0.32), (DEXLANSOPRAZOLE:Peripheral vascular disorder:1.12), (DOFETILIDE:Peripheral vascular disorder:0.29), (EDOXABAN TOSYLATE:Peripheral vascular disorder:0.52), (ELETRIPTAN HYDROBROMIDE:Peripheral vascular disorder:0.13), (EMPAGLIFLOZINLINAGLIPTIN:Peripheral vascular disorder:0.09), (EPLERENONE:Peripheral vascular disorder:0.55), (FLECAINIDE:Peripheral vascular disorder:0.63), (FLUTICASONE FUROATESALMETEROL XINAFOATE:Peripheral vascular disorder:0.11), (FORMOTEROL FUMARATE:Peripheral vascular disorder:0.19), (GADOLINIUM:Peripheral vascular disorder:0.2), (HYDROCHLOROTHIAZIDELISINOPRIL:Peripheral vascular disorder:1.2), (ILOPROST:Peripheral vascular disorder:0.5), (INDAPAMIDEPERINDOPRIL:Peripheral vascular disorder:0.67), (INFLUENZA VIRUS VACCINE:Peripheral vascular disorder:0.15), (LERCANIDIPINE:Peripheral vascular disorder:1.29), (LEVALBUTEROL HYDROCHLORIDE:Peripheral vascular disorder:0.38), (LIDOCAINE HYDROCHLORIDE:Peripheral vascular disorder:0.22), (LISDEXAMFETAMINE DIMESYLATE:Peripheral vascular disorder:0.25), (METAMIZOLE:Peripheral vascular disorder:0), (METAXALONE:Peripheral vascular disorder:0.47), (METRONIDAZOLEMETRONIDAZOLE HYDROCHLORIDE:Peripheral vascular disorder:0.59), (MOXIFLOXACIN HYDROCHLORIDE:Peripheral vascular disorder:1.01), (NADOLOL:Peripheral vascular disorder:0.26), (NITROFURANTOIN:Peripheral vascular disorder:0.65), (NORTRIPTYLINE:Penpheral vascular disorder:0.19), (PANTOPRAZOLE MAGNESIUM:Peripheral vascular disorder:0.71), (PAROXETINEPAROXETINE HYDROCHLORIDE:Peripheral vascular disorder:0.57), (PITAVASTATIN CALCIUM:Peripheral vascular disorder:0.32), (PRAMIPEXOLE:Peripheral vascular disorder:0.61), (RABEPRAZOLE:Peripheral vascular disorder:0.3), (RALOXIFENE HYDROCHLORIDE:Peripheral vascular disorder:0.14), (ROPINIROLE HYDROCHLORIDE:Peripheral vascular disorder:0.15), (SUMATRIPTANSUMATRIPTAN SUCCINATE:Peripheral vascular disorder:0.21), (TERIFLUNOMIDE:Peripheral vascular disorder:0.52), (TIMOLOL:Peripheral vascular disorder:0.29), (TIZANIDINE:Peripheral vascular disorder:0.3), (TRIAMCINOLONE ACETONIDE:Peripheral vascular disorder:0.73), (TRIAMTERENE:Peripheral vascular disorder:0.06), (UMECLIDINIUM BROMIDEVILANTEROL TRIFENATATE:Peripheral vascular disorder:0.36), (VALACYCLOVIR HYDROCHLORIDE:Penpheral vascular disorder:0.63), (VITAMIN B:Peripheral vascular disorder:0.65), (AMIODARONE:Peripheral venous disease:0.54), (ANASTROZOLE:Peripheral venous disease:0.59), (CORTISONEHYDROCORTISONE:Peripheral venous disease:0.32), (DASATINIB:Peripheral venous disease:0.23), (DEXLANSOPRAZOLE:Peripheral venous disease:0.51), (ENOXAPARIN:Peripheral venous disease:0.32), (EVEROLIMUS:Peripheral venous disease:0.52), (IBRUTINIB:Peripheral venous disease:1.03), (INSULIN HUMAN:Penpheral venous disease:0.31), (IRON:Peripheral venous disease:0.08), (RANOLAZINE:Peripheral venous disease:0.35), (SECUKINUMAB:Penpheral venous disease:0.19), (SELEXIPAG:Peripheral venous disease:0.07), (TERIFLUNOMIDE:Peripheral venous disease:0.22), (TOPIRAMATE:Peripheral venous disease:0.4), (TRAZODONE HYDROCHLORIDE:Peripheral venous disease:0.47), (ACARBOSE:Peritoneal dialysis:0.28), (AGALSIDASE BETA:Peritoneal dialysis:0.01), (ALISKIREN:Peritoneal dialysis:0.07), (ATROPINE SULFATEDIPHENOXYLATE HYDROCHLORIDE:Pentoneal dialysis:0.11), (BUDESONIDEFORMOTEROL FUMARATE:Peritoneal dialysis:0.06), (BUSPIRONE HYDROCHLORIDE:Peritoneal dialysis:0.09), (CALCIUMVITAMIN D:Peritoneal dialysis:0.16), (CANDESARTAN CILEXETILHYDROCHLOROTHIAZIDE:Peritoneal dialysis:0.64), (CARVEDILOL PHOSPHATE:Peritoneal dialysis:0.38), (CEFTRIAXONE SODIUM:Peritoneal dialysis:0.57), (CERTOLIZUMAB PEGOL:Peritoneal dialysis:0.24), (CHOLESTYRAMINE:Peritoneal dialysis:0.49), (CYCLOBENZAPRINE HYDROCHLORIDE:Peritoneal dialysis:0.13), (DABIGATRAN:Peritoneal dialysis:0.08), (DASATINIB:Peritoneal dialysis:0.22), (DESLORATADINE:Peritoneal dialysis:0.13), (DEXLANSOPRAZOLE:Peritoneal dialysis:1.23), (DULAGLUTIDE:Peritoneal dialysis:0.04), (EDOXABAN TOSYLATE:Peritoneal dialysis:0.12), (EPLERENONE:Peritoneal dialysis:0.42), (ESZOPICLONE:Peritoneal dialysis:0.38), (EVOLOCUMAB:Peritoneal dialysis:0.48), (FERRIC HYDROXIDE:Peritoneal dialysis:0.13), (FESOTERODINE FUMARATE:Peritoneal dialysis:0.16), (FISH OIL\OMEGA-3 FATTY ACIDS:Peritoneal dialysis:0), (FLECAINIDE:Peritoneal dialysis:0.51), (GLYCOPYRRONIUMINDACATEROL:Peritoneal dialysis:0.1), (HEPARIN CALCIUM:Peritoneal dialysis:0.07), (HYDROCHLOROTHIAZIDEIRBESARTAN:Peritoneal dialysis:0.02), (HYDROCHLOROTHIAZIDELISINOPRIL:Peritoneal dialysis:0.13), (HYDROCHLOROTHIAZIDEVALSARTAN:Peritoneal dialysis:0.65), (HYDROXYZINE PAMOATE:Peritoneal dialysis:0.11), (IBRUTINIB:Peritoneal dialysis:0.55), (ILOPROST:Peritoneal dialysis:0.31), (IMATINIB MESYLATE:Peritoneal dialysis:0.27), (INDAPAMIDEPERINDOPRIL:Peritoneal dialysis:0.3), (IPRATROPIUM:Peritoneal dialysis:0.37), (LACOSAMIDE:Peritoneal dialysis:0.16), (LEDIPASVIRSOFOSBUVIR:Peritoneal dialysis:0.11), (LEVALBUTEROL HYDROCHLORIDE:Peritoneal dialysis:0.31), (LIDOCAINE HYDROCHLORIDE:Peritoneal dialysis:0.11), (LITHIUM CARBONATE:Peritoneal dialysis:0.15), (METAXALONE:Peritoneal dialysis:0.03), (METHADONE HYDROCHLORIDE:Peritoneal dialysis:0.43), (METHYLPREDNISOLONE ACETATE:Peritoneal dialysis:0.26), (MOXIFLOXACIN HYDROCHLORIDE:Peritoneal dialysis:0.46), (NADROPARIN CALCIUM:Peritoneal dialysis:0.03), (NALOXONE HYDROCHLORIDEOXYCODONE HYDROCHLORIDE:Peritoneal dialysis:0.31), (NILOTINIB:Peritoneal dialysis:0.22), (PAROXETINEPAROXETINE HYDROCHLORIDE:Peritoneal dialysis:0.37), (RALOXIFENE HYDROCHLORIDE:Peritoneal dialysis:0.03), (SACUBITRILVALSARTAN:Peritoneal dialysis:0.51), (SODIUM PHOSPHATE, DIBASIC\SODIUM PHOSPHATE, MONOBASIC:Peritoneal dialysis:0.22), (SOTALOL:Peritoneal dialysis:0.96), (SULFAMETHOXAZOLE:Peritoneal dialysis:0.05), (TERAZOSIN HYDROCHLORIDE:Peritoneal dialysis:0.01), (TERIFLUNOMIDE:Peritoneal dialysis:0.02), (TIOTROPIUM:Peritoneal dialysis:0.13), (TIZANIDINE:Peritoneal dialysis:0.19), (TOCILIZUMAB:Peritoneal dialysis:0.11), (TOPIRAMATE:Peritoneal dialysis:1.06), (VALGANCICLOVIR HYDROCHLORIDE:Peritoneal dialysis:0.54), (BISOPROLOL FUMARATE:Pernicious anaemia:0.09), (BUMETANIDE:Pernicious anaemia:0.02), (BUSPIRONE HYDROCHLORIDE:Pernicious anaemia:0.34), (CORTISONEHYDROCORTISONE:Pernicious anaemia:0.04), (DEXLANSOPRAZOLE:Pernicious anaemia:0.71), (DICLOFENAC:Pernicious anaemia:0.24), (DILTIAZEM HYDROCHLORIDE:Pernicious anaemia:1.07), (DIVALPROEX SODIUM:Pernicious anaemia:0.4), (ELETRIPTAN HYDROBROMIDE:Pernicious anaemia:0.01), (EVOLOCUMAB:Pernicious anaemia:0.43), (FINGOLIMOD HYDROCHLORIDE:Pernicious anaemia:0.56), (GLIPIZIDE:Pernicious anaemia:0.08), (IBRUTINIB:Pernicious anaemia:0.99), (LOVASTATIN:Pernicious anaemia:0.22), (METAXALONE:Pernicious anaemia:0.04), (PANTOPRAZOLE:Pernicious anaemia:0.44), (PRAVASTATIN SODIUM:Pernicious anaemia:0.07), (PROGESTERONE:Pernicious anaemia:0.03), (TOCILIZUMAB:Pernicious anaemia:0.09), (VITAMIN B:Pernicious anaemia:0.11), (ALCOHOL:Personality disorder:0.05), (VARENICLINE TARTRATE:Personality disorder:0.2), (CALCIUM:Peyronie's disease:0.84), (DICLOFENAC SODIUM:Peyronie's disease:0.26), (DILTIAZEM HYDROCHLORIDE:Peyronie's disease:0.02), (OXYGEN:Peyronie's disease:0.03), (SPIRONOLACTONE:Peyronie's disease:0.27), (TERIFLUNOMIDE:Peyronie's disease:0.05), (TORSEMIDE:Peyronie's disease:0.16), (EVOLOCUMAB:Phantom pain:0.19), (NAPROXEN:Phantom pain:0.55), (RAMIPRIL:Phantom pain:0.09), (ACETAZOLAMIDE:Pharyngitis:0.02), (ALENDRONIC ACID:Pharyngitis:0.12), (ALUMINUM SILICATE:Pharyngitis:0.01), (AMEZINIUM METILSULFATE:Pharyngitis:0.51), (AMIODARONE HYDROCHLORIDE:Pharyngitis:0.24), (AMOXAPINE:Pharyngitis:0.02), (ANASTROZOLE:Ph aryngitis:1.15), (APREPITANT:Pharyngitis:0.11), (ATOVAQUONE:Pharyngitis:0.01), (AZILSA RTAN KAMEDOXOMIL:Pharyngitis:0.6), (BEPRIDIL:Pharyngitis:0.14), (BISACODYL OR DOCUSATE SODIUM:Pharyngitis:0.41), (BUDESONIDEFORMOTEROL FUMARATE:Pharyngitis:0.03), (BUPRENORPHINE HYDROCHLORIDE:Pharyngitis:0.18), (BUSPIRONE HYDROCHLORIDE:Pharyngitis:0.2), (CALCIUM LACTATE:Pharyngitis:0.16), (CANAKINUMAB:Pharyngitis:0.67), (CANDESARTAN CILEXETILHYDROCHLOROTHIAZIDE:Pharyngitis:0.27), (CEFAZOLIN SODIUM:Pharyngitis:0.11), (CEFTERAM PIVOXIL:Pharyngitis:0.12), (CEPHARANTHINE:Pharyngitis:0.13), (CORTISONEHYDROCO RTISONE:Pharyngitis:0.57), (CYANOCOBALAMINDEXPANTHENOLNIACINAMIDEPY RIDOXINE HYDROCHLORIDE\RIBOFLAVIN 5′-PHOSPHATE SODIUMTHIAMINE HYDROCHLORIDEVITAMIN B COMPLEX:Pharyngitis:0.02), (DACLIZUMAB:Pharyngitis:0), (DASATINIB:Pharyngitis:0.79), (DILTIAZEM:Pharyngitis:0.42), (DILTIAZEM HYDROCHLORIDE:Pharyngitis:0.52), (DIMENHYDRINATE:Pharyngitis:0.16), (DIPHENIDOL HYDROCHLORIDE:Pharyngitis:0.27), (DOCUSATE:Pharyngitis:0.28), (DRONABINOL:Phary ngitis:0.05), (DULOXETINE:Pharyngitis:0.37), (ELETRIPTAN HYDROBROMIDE:Pharyngitis:0.1), (ENOXAPARIN:Pharyngitis:1.26), (ETHYL ICOSAPENTATEICOSAPENT:Pharyngitis:0.03), (ETRETINATE:Pharyngitis:0.8), (EZETIMI BESIMVASTATIN:Pharyngitis:0.03), (FAROPENEM SODIUM:Pharyngitis:0.23), (FERROUS SULFATEFERROUS SULFATE, DRIED:Pharyngitis:1.09), (FLUOXETINEFLUOXETINE HYDROCHLORIDE:Pharyngitis:0.18), (FLUTICASONE FUROATESALMETEROL XINAFOATE:Pharyngitis:0.09), (FLUTICASONE FUROATEVILANTEROL TRIFENATATE:Pharyngitis:0.02), (GLYCOPYRRONIUMINDACATEROL:Pharyngitis:0.02), (GUANABENZ ACETATE:Pharyngitis:0.1), (HYDROXYCHLOROQUINE DIPHOSPHATE:Pharyngitis:0.15), (LABETALOLLABETALOL HYDROCHLORIDE:Pharyngitis:0.22), (LERCANIDIPINE HYDROCHLORIDE:Pharyngitis:0.06), (LETROZOLE:Pharyngitis:0.03), (LEVOCABASTINE HYDROCHLORIDE:Pharyngitis:0.19), (LIDOCAINEPRILOCAINE:Pharyngitis:0.13), (LITHI UM CARBONATE:Pharyngitis:0.13), (LOVASTATIN:Pharyngitis:0.6), (LYSOZYME HYDROCHLORIDE:Pharyngitis:0.23), (METHADONE HYDROCHLORIDE:Pharyngitis:0.24), (METHENOLONE ENANTHATE:Pharyngitis:0.26), (METHYLPREDNISOLONE ACETATE:Pharyngitis:0.17), (NEBIVOLOL:Pharyngitis:0.35), (NILOTINIB:Pharyngitis:1.1), (O XYBUTYNIN CHLORIDE:Pharyngitis:0), (PANTOPRAZOLE MAGNESIUM:Pharyngitis:0.44), (PAROXETINE:Pharyngitis:0.19), (PAROXETINEPAROXE TINE HYDROCHLORIDE:Pharyngitis:0.32), (PAZOPANIB HYDROCHLORIDE:Pharyngitis:0.26), (PHENOBARBITAL:Pharyngitis:0.1), (PHENYTOIN:P haryngitis:0.05), (PIMOBENDAN:Pharyngitis:0.01), (POLYETHYLENE GLYCOLS:Pharyngitis:0.84), (POTASSIUM:Pharyngitis:0.72), (POTASSIUM CITRATETRISODIUM CITRATE DIHYDRATE:Pharyngitis:0.35), (PRIMIDONE:Pharyngitis:0.54), (PROGESTERONE:Pharyngit is:0.5), (PYRIDOXINE:Pharyngitis:0.05), (RANOLAZINE:Pharyngitis:0.18), (RIZATRIPTAN BENZOATE:Pharyngitis:0.12), (ROFLUMILAST:Pharyngitis:0.21), (SACUBITRILVALSART AN:Pharyngitis:0.26), (SCOPOLAMINE:Pharyngitis:0.17), (SITAGLIPTIN:Pharyngitis:0.02), (S ODIUM OXYBATE:Pharyngitis:0.14), (SOTALOL:Pharyngitis:0.12), (SUPLATAST TOSILATE:Pharyngitis:0.21), (TAMSULOSIN:Pharyngitis:1.22), (TEMAZEPAM:Pharyngitis:0.35), (TERIFLUNOMIDE:Pharyngitis:0.17), (THALIDOMIDE:Pharyngitis:0.75), (TORSEMIDE: Pharyngitis:0.62), (VARENICLINE TARTRATE:Pharyngitis:0.36), (VITAMIN B:Pharyngitis:0.28), (WARFARIN POTASSIUM:Pharyngitis:0.14), (ANASTROZOLE:Phlebitis:0.18), (BUPROPION HYDROCHLORIDE:Phlebitis:0.02), (DICLOFENAC:Phlebitis:0.33), (ENALAPRIL:Phlebitis:0.5), (EVOLOCUMAB:Phlebitis:0.26), (FERROUS SULFATEFERROUS SULFATE, DRIED:Phlebitis:0.09), (ISOSORBIDE MONONITRATE:Phlebitis:0.15), (SACUBITRILVALSARTAN:Phlebitis:0.43), (SOTALOL:Ph lebitis:0.06), (TOFACITINIB CITRATE:Phlebitis:0.37), (VERAPAMIL HYDROCHLORIDE:Phlebitis:0.15), (AMIODARONE:Pituitary-dependent Cushing's syndrome:0.49), (CANAGLIFLOZIN:Pituitary-dependent Cushing's syndrome:0.15), (CELECOXIB:Pituitary-dependent Cushing's syndrome:0.24), (DIGOXIN:Pituitary-dependent Cushing's syndrome:0.73), (DILTIAZEM:Pituitary-dependent Cushing's syndrome:0.31), (EVOLOCUMAB:Pituitary-dependent Cushing's syndrome:0.65), (IBRUTINIB:Pituitary-dependent Cushing's syndrome:0.1), (PRAVASTATIN:Pituitary-dependent Cushing's syndrome:0.21), (RANOLAZINE:Pituitary-dependent Cushing's syndrome:0.14), (TAMSULOSIN:Pituitary-dependent Cushing's syndrome:0.1), (PYRAZINAMIDE:Plantar fasciitis:0.59), (.BETA.-CAROTENE:Plasma cell myeloma:1.08), (.BETA.-HYDROXYISOVALERIC ACIDARGININEGLUTAMINE:Plasma cell myeloma:0.71), (ABOBOTULINUMTOXINA:Plasma cell myeloma:0.42), (ACAMPROSATE:Plasma cell myeloma:0.26), (ACEMETACIN:Plasma cell myeloma:0.08), (ACETAMINOPHENAPRONALIDECAFFEINEPROPYPHENAZONE:Plas ma cell myeloma:0.41), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Plasma cell myeloma:0.12), (ACETAMINOPHEN\CAFFEINECARISOPRODOLDICLOFENAC:Plasma cell myeloma:0.43), (ACETAMINOPHEN\CAFFEINECARISOPRODOLDICLOFENAC SODIUM:Plasma cell myeloma:0.23), (ACETAMINOPHEN\CAFFEINECODEINE PHOSPHATE:Plasma cell myeloma:0), (ACETAMINOPHENCHLORPHENIRAMINE MALEATEPSEUDOEPHEDRINE HYDROCHLORIDE:Plasma cell myeloma:0.73), (ACETAMINOPHENCHLORZOXAZONE:Plasma cell myeloma:1), (ACETAMINOPHENDICHLORALPHENAZONEISOMETHEPTENE HYDROCHLORIDE:Plasma cell myeloma:0.47), (ACETAMINOPHENMETHOCARBAMOL:Plasma cell myeloma:1.41), (ACETAMINOPHENPHENYLEPHRINE HYDROCHLORIDE:Plasma cell myeloma:0.18), (ACLARUBICIN HYDROCHLORIDE:Plasma cell myeloma:0.14), (ACRIVASTINEPSEUDOEPHEDRINE HYDROCHLORIDE:Plasma cell myeloma:0.82), (ADAPALENEBENZOYL PEROXIDE:Plasma cell myeloma:0.2), (ADEFOVIR:Plasma cell myeloma:0.03), (ADENINE:Plasma cell myeloma:0.37), (ADO-TRASTUZUMAB EMTANSINE:Plasma cell myeloma:2.06), (AGALSIDASE BETA:Plasma cell myeloma:2.58), (AGOMELATINE:Plasma cell myeloma:0.72), (ALBUTEROLGUAIFENESIN:Plasma cell myeloma:0.7), (ALBUTEROLIPRATROPIUM BROMIDE:Plasma cell myeloma:0.76), (ALCOHOLALUMINUM CHLORIDE:Plasma cell myeloma:0.21), (ALDESLEUKIN:Plasma cell myeloma:0.45), (ALECTINIB:Plasma cell myeloma:0.19), (ALECTINIB HYDROCHLORIDE:Plasma cell myeloma:0.11), (ALGINIC ACID:Plasma cell myeloma:0.13), (ALISKIREN HEMIFUMARATEVALSARTAN:Plasma cell myeloma:1.33), (ALISKIRENHYDROCHLOROTHIAZIDE:Plasma cell myeloma:0.46), (ALOGLIPTIN:Plasma cell myeloma:0.8), (ALOSETRON HYDROCHLORIDE:Plasma cell myeloma:0.18), (ALPHA-TOCOPHEROLASCORBIC ACIDCHOLECALCIFEROLCOPPERCYANOCOBALAMINFOLIC ACIDLUTEIN\OMEGA-3 FATTY ACIDSPYRIDOXINE HYDROCHLORIDETAURINEVITAMIN A PALMITATEZEAXANTHINZINC PICOLINATE:Plasma cell myeloma:0.23), (ALPROSTADIL ALFADEX:Plasma cell myeloma:0.02), (ALUMINUM CHLORIDE:Plasma cell myeloma:0.28), (AMDINOCILLIN PIVOXIL HYDROCHLORIDE:Plasma cell myeloma:0.24), (AMINOSALICYLIC ACID:Plasma cell myeloma:0.39), (AMLODIPINELOSARTAN:Plasma cell myeloma:0.41), (ANAGLIPTIN:Plasma cell myeloma:0.18), (ANAGRELIDE:Plasma cell myeloma:1.12), (ANGIOTENSIN II:Plasma cell myeloma:0.04), (APRINDINE HYDROCHLORIDE:Plasma cell myeloma:0.36), (ARCTOSTAPHYLOS UVA-URSI LEAFURTICA DIOICA:Plasma cell myeloma:0.57), (ARFORMOTEROL:Plasma cell myeloma:0.1), (ARNICA MONTANA:Plasma cell myeloma:0.03), (AROTINOLOL:Plasma cell myeloma:0.07), (ASCORBIC ACID/CHROMIUM/SELENIUM/VANADYL SULFATE:Plasma cell myeloma:1.16), (ASCORBIC ACIDCHONDROITIN SULFATE (BOVINE)GLUCOSAMINE SULFATEMANGANESE:Plasma cell myeloma:0.21), (ASCORBIC ACIDFERROUS SULFATEFOLIC ACIDPYRIDOXINE HYDROCHLORIDE\RIBOFLAVINTHIAMINE MONONITRATE:Plasma cell myeloma:0.28), (ASCORBIC ACIDIRON:Plasma cell myeloma:0.31), (ASCORBIC ACIDVITAMIN B COMPLEX:Plasma cell myeloma:0.04), (ASPIRINBUTALBITALCAFFEINECODEINE PHOSPHATE:Plasma cell myeloma:0.46), (ASPIRIN\CAFFEINESALICYLAMIDE:Plasma cell myeloma:0.95), (ASPIRINCLOPIDOGREL:Plasma cell myeloma:1.09), (ASPIRINDIHYDROXYALUMINUM AMINOACETATE ANHYDROUSMAGNESIUM CARBONATE:Plasma cell myeloma:1.6), (AZITHROMYCIN OR METHOTREXATE SODIUM:Plasma cell myeloma:1.59), (BACITRACINNEOMYCIN SULFATEPOLYMYXIN B SULFATE:Plasma cell myeloma:0.75), (BALSALAZIDE:Plasma cell myeloma:0.92), (BALSAM PERUBISMUTH HYDROXIDEBISMUTH SUBGALLATEBORIC ACIDZINC OXIDE:Plasma cell myeloma:0.01), (BAMIFYLLINE HYDROCHLORIDE:Plasma cell myeloma:1.22), (BELATACEPT:Plasma cell myeloma:1.18), (BENZALKONIUM CHLORIDE:Plasma cell myeloma:0.21), (BENZYDAMINE:Plasma cell myeloma:0.07), (BERAPROST:Plasma cell myeloma:0.55), (BERAPROST SODIUM:Plasma cell myeloma:1.31), (BERBERINE:Plasma cell myeloma:0.19), (BETAMETHASONE DIPROPIONATEBETAMETHASONE SODIUM PHOSPHATE:Plasma cell myeloma:0.19), (BETAMETHASONE DIPROPIONATECALCIPOTRIENE:Plasma cell myeloma:1.74), (BETAMETHASONE DIPROPIONATECALCIPOTRIENE HYDRATE:Plasma cell myeloma:1.21), (BETAMETHASONE DIPROPIONATECLOTRIMAZOLEGENTAMICIN SULFATE:Plasma cell myeloma:0.17), (BETAMETHASONECALCIPOTRIENE:Plasma cell myeloma:1.98), (BETAMIPRONPANIPENEM:Plasma cell myeloma:0.49), (BETHANECHOL:Plasma cell myeloma:0.73), (BEXAROTENE:Plasma cell myeloma:0.68), (ACIDOPHILUS:Plasma cell myeloma:1.11), (BILASTINE:Plasma cell myeloma:1.97), (BISACODYLGLYCERINMINERAL OILSODIUM PHOSPHATE, DIBASIC\SODIUM PHOSPHATE, MONOBASIC:Plasma cell myeloma:1.1), (BISMUTH SUBCITRATERANITIDINE HYDROCHLORIDESUCRALFATE:Plasma cell myeloma:0.01), (BLESELUMAB:Plasma cell myeloma:0.25), (BLONANSERIN:Plasma cell myeloma:0.16), (BOSUTINIB:Plasma cell myeloma:0.7), (BOSUTINIB MONOHYDRATE:Plasma cell myeloma:0.66), (BRIMONIDINE TARTRATEDORZOLAMIDE HYDROCHLORIDETIMOLOL MALEATE:Plasma cell myeloma:0.23), (BUCILLAMINE:Plasma cell myeloma:1.4), (BUFORMIN:Plasma cell myeloma:0.41), (BUPRENORPHINENALOXONE:Plasma cell myeloma:0.19), (BUPROPION HYDROBROMIDE:Plasma cell myeloma:0.59), (BUTALBITAL:Plasma cell myeloma:1.1), (BUTORPHANOL:Plasma cell myeloma:0.58), (CABAZITAXEL:Plasma cell myeloma:0.87), (CABOZANTINIB:Plasma cell myeloma:0.84), (CAFFEINEDIPYRONEORPHENADRINE:Plasma cell myeloma:0.76), (CAFFEINEERGOTAMINE TARTRATE:Plasma cell myeloma:0.04), (CAFFEINEISOMETHEPTENEMETHIMAZOLE:Plasma cell myeloma:0.19), (CAJUPUT OILEUCALYPTUS OILPEPPERMINT OIL:Plasma cell myeloma:0.83), (CALCIUM BICARBONATE:Plasma cell myeloma:0.71), (CALCIUM CARBIMIDE:Plasma cell myeloma:0.15), (CALCIUM CARBONATEETIDRONATE DISODIUM:Plasma cell myeloma:0.93), (CALCIUM CHLORIDEDEXTROSE\POTASSIUM CHLORIDE\SODIUM ACETATE\SODIUM CHLORIDE:Plasma cell myeloma:0.22), (CALCIUM GLYCEROPHOSPHATE:Plasma cell myeloma:0.57), (CALCIUMCHOLECALCIFEROLCOPPERMAGNESIUMMANGANESE ZINC:Plasma cell myeloma:0.37), (CALCIUMCHOLECALCIFEROLMAGNESIUM\ZINC:Plasma cell myeloma:0.13), (CAMPHORDIPHENHYDRAMINE\ZINC OXIDE:Plasma cell myeloma:1.06), (CANAKINUMAB:Plasma cell myeloma:1.74), (CAPSICUM:Plasma cell myeloma:0.32), (CARBOMER 934GLYCERINMETHYLPARABENPROPYLPARABEN\SODIUM HYDROXIDE:Plasma cell myeloma:0.02), (CARBOXYMETHYLCELLULOSE SODIUMGELATINPECTIN:Plasma cell myeloma:0.07), (CARBOXYMETHYLCELLULOSE SODIUMGLYCERINPOLYSORBATE 80:Plasma cell myeloma:1.18), (CARTEOLOL:Plasma cell myeloma:0.36), (CEFMETAZOLE:Plasma cell myeloma:0.12), (CEFMETAZOLE SODIUM:Plasma cell myeloma:0.36), (CEFPIROME SULFATE:Plasma cell myeloma:0.25), (CEFTERAM PIVOXIL:Plasma cell myeloma:0.18), (CEPHARANTHINE:Plasma cell myeloma:0.69), (CERIVASTATIN SODIUM:Plasma cell myeloma:1.32), (CHAMOMILE:Plasma cell myeloma:0.05), (CHLORMADINONE ACETATE:Plasma cell myeloma:0.67), (CHLORMADINONE ACETATEETHINYL ESTRADIOL:Plasma cell myeloma:0.77), (CHLORPROTHIXENE:Plasma cell myeloma:0.18), (CHOLECALCIFEROLGENISTEINZINC GLYCINATE:Plasma cell myeloma:0.43), (CHOLINE BITARTRATECYSTEINEINOSITOLMAMMAL LIVER:Plasma cell myeloma:0.06), (CHONDROITIN SULFATE (BOVINE)DIMETHYL SULFONEGLUCOSAMINE SULFATE:Plasma cell myeloma:0.25), (CICLOPIROX OLAMINE:Plasma cell myeloma:1.16), (CIPROFLOXACINDEXAMETHASONE:Plasma cell myeloma:0.61), (CISAPRIDE:Plasma cell myeloma:0.77), (CLENBUTEROL HYDROCHLORIDE:Plasma cell myeloma:0.48), (CLIKSTAR:Plasma cell myeloma:1.42), (CLOFARABINE:Plasma cell myeloma:0.51), (CLOFAZIMINE:Plasma cell myeloma:0.06), (CLOSTRIDIUM TETANICLOSTRIDIUM TETANI TOXOID ANTIGEN (FORMALDEHYDE INACTIVATED)DIPHTHERIAECORYNEBACTERIUM DIPHTHERIAE TOXOID ANTIGEN (FORMALDEHYDE INACTIVATED):Plasma cell myeloma:0.66), (CLOXACILLIN SODIUM:Plasma cell myeloma:0.44), (CLOXAZOLAM:Plasma cell myeloma:1.11), (COBICISTATELVITEGRAVIREMTRICITABINETENOFOVIR ALAFENAMIDE FUMARATE:Plasma cell myeloma:0.75), (COBICISTATELVITEGRAVIREMTRICITABINETENOFOVIR DISOPROXIL FUMARATE:Plasma cell myeloma:0.82), (CODEINE PHOSPHATEDICLOFENAC SODIUM:Plasma cell myeloma:0.23), (COPPER OR LEVONORGESTREL:Plasma cell myeloma:0.01), (CORTICOTROPIN:Plasma cell myeloma:2.07), (COUMARINTROXERUTIN:Plasma cell myeloma:0.59), (CREATININE:Plasma cell myeloma:0.1), (CROMOLYN:Plasma cell myeloma:0.16), (CYANOCOBALAMINDICLOFENAC SODIUMPYRIDOXINE HYDROCHLORIDETHIAMINE MONONITRATE:Plasma cell myeloma:0.14), (CYANOCOBALAMINLIDOCAINE HYDROCHLORIDEPYRIDOXINE HYDROCHLORIDETHIAMINE HYDROCHLORIDE:Plasma cell myeloma:0.49), (CYANOCOBALAMINPYRIDOXINE HYDROCHLORIDE\RIBOFLAVIN\SODIUM PANTOTHENATETHIAMINE HYDROCHLORIDEVITAMIN B COMPLEX:Plasma cell myeloma:0.26), (CYANOCOBALAMINPYRIDOXINE HYDROCHLORIDETHIAMINE DISULFIDE:Plasma cell myeloma:0.66), (CYCLOPHOSPHAMIDECYTARABINEDEXAMETHASONEDOXORUBI CINMETHOTREXATEVINCRISTINE:Plasma cell myeloma:0.15), (CYPROTERONE:Plasma cell myeloma:0.6), (CYSTEINE:Plasma cell myeloma:0.51), (DABRAFENIB:Plasma cell myeloma:1.34), (DARIFENACIN:Plasma cell myeloma:0.29), (DASABUVIR:Plasma cell myeloma:0.03), (DASABUVIR SODIUM MONOHYDRATEOMBITASVIR HEMINONAHYDRATEPARITAPREVIR DIHYDRATERITONAVIR:Plasma cell myeloma:0.17), (DEFERIPRONE:Plasma cell myeloma:0.77), (DEFLAZACORT:Plasma cell myeloma:2.29), (DEGARELIX:Plasma cell myeloma:0.98), (DEMECLOCYCLINE:Plasma cell myeloma:0.27), (DENOPAMINE:Plasma cell myeloma:0.12), (DEPRODONE PROPIONATE:Plasma cell myeloma:0.02), (DEXAMETHASONE SODIUM PHOSPHATEMOXIFLOXACIN HYDROCHLORIDE:Plasma cell myeloma:0.11), (DEXAMETHASONENEOMYCIN SULFATE:Plasma cell myeloma:0.03), (DEXMEDETOMIDINE HYDROCHLORIDE:Plasma cell myeloma:0.83), (DEXTRAN SULFATE SODIUM SALT:Plasma cell myeloma:0.59), (DEXTROMETHORPHAN HYDROBROMIDEQUINIDINE SULFATE:Plasma cell myeloma:0.77), (DICHLORPHENAMIDE:Plasma cell myeloma:0.35), (DICLOFENAC DIETHYLAMINE:Plasma cell myeloma:0.41), (DIETARY SUPPLEMENTFLAVOCOXID:Plasma cell myeloma:0.39), (DIHYDROCODEINE PHOSPHATE:Plasma cell myeloma:0.62), (DIMEMORFAN:Plasma cell myeloma:0.85), (DIPHENHYDRAMINE HYDROCHLORIDEPHENYLEPHRINE HYDROCHLORIDE:Plasma cell myeloma:0.27), (DIPHENIDOL HYDROCHLORIDE:Plasma cell myeloma:1.12), (DIPYRONEPROMETHAZINE HYDROCHLORIDE:Plasma cell myeloma:1.61), (DOCUSATE SODIUMSENNOSIDE ASENNOSIDE B:Plasma cell myeloma:0.65), (DOXYLAMINE SUCCINATEPYRIDOXINE HYDROCHLORIDE:Plasma cell myeloma:0.48), (DUPILUMAB:Plasma cell myeloma:0.19), (DYPHYLLINE:Plasma cell myeloma:0.05), (ECABET:Plasma cell myeloma:0.61), (ECABET SODIUM:Plasma cell myeloma:0.68), (ECALLANTIDE:Plasma cell myeloma:0.48), (EDARAVONE:Plasma cell myeloma:0.79), (ELBASVIRGRAZOPREVIR:Plasma cell myeloma:0.25), (ELECTROLYTES NOSPOLYETHYLENE GLYCOL 3350:Plasma cell myeloma:0.02), (EMPAGLIFLOZINMETFORMIN HYDROCHLORIDE:Plasma cell myeloma:0.25), (EMTRICITABINERILPIVIRINETENOFOVIR DISOPROXIL FUMARATE:Plasma cell myeloma:0.35), (EPACADOSTAT:Plasma cell myeloma:0.02), (EPALRESTAT:Plasma cell myeloma:0.18), (EPHEDRINE HYDROCHLORIDE:Plasma cell myeloma:0.08), (EPHEDRINE HYDROCHLORIDEGUAIFENESIN:Plasma cell myeloma:0.72), (EPRAZINONE:Plasma cell myeloma:0.65), (EPROSARTAN MESYLATEHYDROCHLOROTHIAZIDE:Plasma cell myeloma:0.05), (ERGOLOID MESYLATES:Plasma cell myeloma:0.47), (ERIBULIN:Plasma cell myeloma:0.28), (ERIBULIN MESYLATE:Plasma cell myeloma:0.71), (ESTRADIOL BENZOATE:Plasma cell myeloma:1.18), (ESTROGENS, ESTERIFIED:Plasma cell myeloma:0.24), (ESTROGENSMETHYLTESTOSTERONE:Plasma cell myeloma:0.74), (ETHINYL ESTRADIOLGESTODENE:Plasma cell myeloma:0.29), (FELBAMATE:Plasma cell myeloma:0.12), (FERRIC CITRATE ANHYDROUS:Plasma cell myeloma:0.34), (FERRIC HYDROXIDE:Plasma cell myeloma:0.91), (FERRIC SODIUM CITRATE:Plasma cell myeloma:0.08), (FERROUS BISGLYCINATE:Plasma cell myeloma:1.27), (FERROUS CITRATE\SODIUM:Plasma cell myeloma:0.04), (FLOMOXEF:Plasma cell myeloma:0.57), (FLUNARIZINE HYDROCHLORIDE:Plasma cell myeloma:1.25), (FLUORESCEIN:Plasma cell myeloma:0.27), (FLUPHENAZINE:Plasma cell myeloma:0.21), (FLURANDRENOLIDE:Plasma cell myeloma:0.64), (FLUTICASONE PROPIONATEFORMOTEROL FUMARATE:Plasma cell myeloma:1.14), (FOLIC ACIDIRONMINERALSVITAMINS:Plasma cell myeloma:0.62), (FOSFOMYCIN CALCIUM:Plasma cell myeloma:0.46), (FRANGULA PURSHIANA BARK:Plasma cell myeloma:0), (FUDOSTEINE:Plasma cell myeloma:0.63), (FURSULTIAMINE:Plasma cell myeloma:0.57), (FUSIDATE SODIUM:Plasma cell myeloma:0.38), (GABEXATE MESYLATE:Plasma cell myeloma:0.41), (GADOTERATE MEGLUMINE:Plasma cell myeloma:0.05), (GADOXETATE DISODIUM:Plasma cell myeloma:0.1), (GENISTEIN:Plasma cell myeloma:0.03), (GENTIAN VIOLET:Plasma cell myeloma:0.89), (GILTERITINIB:Plasma cell myeloma:0.27), (GLIMEPIRIDEROSIGLITAZONE MALEATE:Plasma cell myeloma:0.57), (GLUCOSAMINE HYDROCHLORIDE:Plasma cell myeloma:0.29), (GLYCOPYRROLATEINDACATEROL MALEATE:Plasma cell myeloma:1.67), (GUAIFENESINPHENYLEPHRINE HYDROCHLORIDE:Plasma cell myeloma:0.76), (GUANABENZ ACETATE:Plasma cell myeloma:0.14), (GUSPERIMUS TRIHYDROCHLORIDE:Plasma cell myeloma:0.06), (HERBALSHOMEOPATHICSGOLDENSEAL:Plasma cell myeloma:0.3), (HONEY:Plasma cell myeloma:0.32), (HUMAN COLOSTRUM:Plasma cell myeloma:0.16), (HYDROCORTISONE PROBUTATE:Plasma cell myeloma:0.69), (HYDROCORTISONENEOMYCIN SULFATEPOLYMYXIN B SULFATE:Plasma cell myeloma:0.44), (HYDROXOCOBALAMIN ACETATE:Plasma cell myeloma:0.42), (HYDROXYPROPYL CELLULOSE:Plasma cell myeloma:1.01), (IDARUCIZUMAB:Plasma cell myeloma:0.3), (IFENPRODIL:Plasma cell myeloma:0.84), (IGURATIMOD:Plasma cell myeloma:0.89), (IMIDAFENACIN:Plasma cell myeloma:0.39), (IMIDAPRIL HYDROCHLORIDE:Plasma cell myeloma:0.07), (IMIPRAMINE PAMOATE:Plasma cell myeloma:0.35), (INDACATEROL MALEATE:Plasma cell myeloma:1.14), (INFLIXIMAB-DYYB:Plasma cell myeloma:1.31), (INSULIN ASPARTINSULIN DEGLUDEC:Plasma cell myeloma:0.36), (INSULIN PORKINSULIN PURIFIED PORK:Plasma cell myeloma:1.36), (INTERFERON ALFA-N1:Plasma cell myeloma:0.05), (INTERFERON BETA:Plasma cell myeloma:0.4), (INTERFERON GAMMA-1B:Plasma cell myeloma:2.14), (IODINE\POTASSIUM IODIDE:Plasma cell myeloma:0.64), (IPRAGLIFLOZIN:Plasma cell myeloma:0.27), (IPRAGLIFLOZIN L-PROLINE:Plasma cell myeloma:0.72), (IRON POLYMALTOSE:Plasma cell myeloma:0.85), (ISOFLAVONES SOY:Plasma cell myeloma:0.63), (ISOLEUCINELEUCINEVALINE:Plasma cell myeloma:0.08), (ISONIAZIDPYRAZINAMIDERIFAMPIN:Plasma cell myeloma:0.28), (ISOPROPYL ALCOHOL:Plasma cell myeloma:0.06), (IVACAFTOR:Plasma cell myeloma:0.17), (IXABEPILONE:Plasma cell myeloma:0.04), (KALLIDINOGENASE:Plasma cell myeloma:0.75), (KETAMINE HYDROCHLORIDE:Plasma cell myeloma:0.64), (LACTIC ACID:Plasma cell myeloma:0.34), (LANOLINMINERAL OILPETROLATUM:Plasma cell myeloma:0.47), (LANOLINPETROLATUM:Plasma cell myeloma:0.42), (LANREOTIDE:Plasma cell myeloma:1.33), (LANREOTIDE ACETATE:Plasma cell myeloma:0.5), (LAPATINIB:Plasma cell myeloma:0.09), (LAVENDER OIL:Plasma cell myeloma:0.37), (LESINURAD:Plasma cell myeloma:0.39), (LETROZOLERIBOCICLIB:Plasma cell myeloma:0.34), (LEVOCARNITINE HYDROCHLORIDE:Plasma cell myeloma:0.69), (LEVOMEFOLIC ACIDMETHYLCOBALAMINTURMERIC:Plasma cell myeloma:0.15), (LEVOMILNACIPRAN:Plasma cell myeloma:0.26), (LEVOSIMENDAN:Plasma cell myeloma:0.56), (LIPASE:Plasma cell myeloma:0.28), (LOMITAPIDE MESYLATE:Plasma cell myeloma:1.86), (LORCASERIN HYDROCHLORIDE:Plasma cell myeloma:0.75), (LUMACAFTOR:Plasma cell myeloma:0), (LUTETIUM LU-177:Plasma cell myeloma:1.04), (LYSOZYME HYDROCHLORIDE:Plasma cell myeloma:0.92), (MAGNESIUM ASPARTATE HYDROCHLORIDE:Plasma cell myeloma:1.57), (MAGNESIUM GLUCEPTATE:Plasma cell myeloma:0.09), (MAGNESIUM GLYCINATE:Plasma cell myeloma:0.41), (MAXACALCITOL:Plasma cell myeloma:1.57), (MECLIZINE MONOHYDROCHLORIDENIACIN:Plasma cell myeloma:0.59), (MECLOZINE DIHYDROCHLORIDE:Plasma cell myeloma:0.82), (MEPOLIZUMAB:Plasma cell myeloma:1.82), (METFORMIN HYDROCHLORIDESAXAGLIPTIN:Plasma cell myeloma:1.56), (METHENOLONE ENANTHATE:Plasma cell myeloma:1.71), (METHYLPREDNISOLONE OR METHYLPREDNISOLONE ACETATE:Plasma cell myeloma:0.04), (METRELEPTIN:Plasma cell myeloma:0.26), (METRONIDAZOLE BENZOATE:Plasma cell myeloma:0.08), (MIGLUSTAT:Plasma cell myeloma:0.47), (MILNACIPRAN:Plasma cell myeloma:0.48), (MILRINONE LACTATE:Plasma cell myeloma:0.01), (MITIGLINIDE CALCIUM HYDRATEVOGLIBOSE:Plasma cell myeloma:0.55), (NABILONE:Plasma cell myeloma:0.97), (NABIXIMOLS:Plasma cell myeloma:0.62), (NADIFLOXACIN:Plasma cell myeloma:0.32), (NAFAMOSTAT MESYLATE:Plasma cell myeloma:0.21), (NALBUPHINE HYDROCHLORIDE:Plasma cell myeloma:1.55), (NALOXONE HYDROCHLORIDEPENTAZOCINE HYDROCHLORIDE:Plasma cell myeloma:0.11), (NAPHAZOLINE HYDROCHLORIDEPHENIRAMINE MALEATE:Plasma cell myeloma:0.18), (NATURAL LATEX RUBBER:Plasma cell myeloma:0.28), (NELARABINE:Plasma cell myeloma:0.38), (NEOMYCINPOLYMYXIN:Plasma cell myeloma:0.26), (NILVADIPINE:Plasma cell myeloma:0.17), (NIRAPARIB:Plasma cell myeloma:1.66), (OBETICHOLIC ACID:Plasma cell myeloma:0.28), (OBINUTUZUMAB:Plasma cell myeloma:1.03), (OCRELIZUMAB:Plasma cell myeloma:0.57), (OFATUMUMAB:Plasma cell myeloma:0.9), (OK-432:Plasma cell myeloma:0.02), (OLAPARIB:Plasma cell myeloma:0.59), (OLODATEROL:Plasma cell myeloma:0.29), (OLODATEROL HYDROCHLORIDE:Plasma cell myeloma:0.76), (OMEGA-6 FATTY ACIDS:Plasma cell myeloma:0.59), (OSIMERTINIB:Plasma cell myeloma:1.11), (OXANDROLONE:Plasma cell myeloma:0.56), (OXITRIPTAN:Plasma cell myeloma:0.57), (OXYBUTYNINOXYBUTYNIN CHLORIDE:Plasma cell myeloma:2.08), (OXYTETRACYCLINE:Plasma cell myeloma:0.1), (PALIPERIDONE PALMITATE:Plasma cell myeloma:0.29), (PARATHYROID HORMONE:Plasma cell myeloma:1.18), (PARSLEY:Plasma cell myeloma:0.63), (PASIREOTIDE:Plasma cell myeloma:0.77), (PEGASPARGASE:Plasma cell myeloma:0.6), (PEGINTERFERON BETA-1A:Plasma cell myeloma:1.76), (PEGZEREPOETIN ALFA:Plasma cell myeloma:0.75), (PEMETREXED DISODIUM:Plasma cell myeloma:1.78), (PENTAZOCINE LACTATE:Plasma cell myeloma:0.55), (PEPPERMINT:Plasma cell myeloma:0.12), (PHENOBARBITAL SODIUM:Plasma cell myeloma:0.85), (PHENTERMINE HYDROCHLORIDETOPIRAMATE:Plasma cell myeloma:0.83), (PHENYLBUTAZONE:Plasma cell myeloma:0.06), (PHENYLEPHRINE HYDROCHLORIDETROPICAMIDE:Plasma cell myeloma:0.2), (PIMOBENDAN:Plasma cell myeloma:1.2), (PINAVERIUM BROMIDE:Plasma cell myeloma:0.97), (PIRENZEPINE:Plasma cell myeloma:0.27), (PITOFENONE:Plasma cell myeloma:0.6), (PIZOTYLINE:Plasma cell myeloma:0.47), (PLASMA PROTEIN FRACTION (HUMAN):Plasma cell myeloma:0.29), (PLECANATIDE:Plasma cell myeloma:0.12), (POLYETHYLENE GLYCOL 400:Plasma cell myeloma:0.89), (POLYMYXIN B SULFATE:Plasma cell myeloma:0.61), (PORFIMER SODIUM:Plasma cell myeloma:0.19), (POTASSIUM CHLORIDE\SODIUM CHLORIDE\SODIUM LACTATE:Plasma cell myeloma:0.55), (POTASSIUM CITRATETRISODIUM CITRATE DIHYDRATE:Plasma cell myeloma:1.39), (PREDNISOLONE VALERATE ACETATE:Plasma cell myeloma:0.52), (PREGNENOLONE:Plasma cell myeloma:0.85), (PROBUCOL:Plasma cell myeloma:0.59), (PROCAINE:Plasma cell myeloma:0.14), (PROCYCLIDINE:Plasma cell myeloma:0.04), (PROPATYL NITRATE:Plasma cell myeloma:1.08), (PROTRIPTYLINE HYDROCHLORIDE:Plasma cell myeloma:2.11), (PRUCALOPRIDE:Plasma cell myeloma:0.03), (PSORALEN:Plasma cell myeloma:0.61), (PYRIDOXAL PHOSPHATE:Plasma cell myeloma:0.92), (QUINACRINE:Plasma cell myeloma:1.62), (RADIUM RA-223 DICHLORIDE:Plasma cell myeloma:0.04), (RAMUCIRUMAB:Plasma cell myeloma:1.05), (RED CLOVER:Plasma cell myeloma:0.34), (RED YEAST:Plasma cell myeloma:0.31), (RESLIZUMAB:Plasma cell myeloma:0.2), (RIBOCICLIB:Plasma cell myeloma:0.19), (RIMABOTULINUMTOXINB:Plasma cell myeloma:0.05), (RISEDRONIC ACID:Plasma cell myeloma:0.91), (ROPIVACAINE HYDROCHLORIDE:Plasma cell myeloma:0.07), (SAPROPTERIN DIHYDROCHLORIDE:Plasma cell myeloma:0.18), (SARPOGRELATE HYDROCHLORIDE:Plasma cell myeloma:0.69), (SELENIUM SULFIDE:Plasma cell myeloma:0.91), (SELEXIPAG:Plasma cell myeloma:0.78), (SENNOSIDE B:Plasma cell myeloma:0.2), (SERRAPEPTASE:Plasma cell myeloma:1.08), (SERTACONAZOLE NITRATE:Plasma cell myeloma:0.29), (SIBUTRAMINE HYDROCHLORIDE:Plasma cell myeloma:1.47), (SIPULEUCEL-T:Plasma cell myeloma:1.43), (SODIUM FLUORIDEVITAMINS:Plasma cell myeloma:0.14), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Plasma cell myeloma:1.04), (SOMATOSTATIN:Plasma cell myeloma:0.35), (STRONTIUM CHLORIDE:Plasma cell myeloma:0.15), (STRONTIUM RANELATE:Plasma cell myeloma:0.02), (SUCCINYLCHOLINE CHLORIDE:Plasma cell myeloma:0.17), (SULFAMERAZINETRIMETHOPRIM:Plasma cell myeloma:0.38), (SULTOPRIDE HYDROCHLORIDE:Plasma cell myeloma:0.79), (TAFAMIDIS MEGLUMINE:Plasma cell myeloma:0.77), (TALIGLUCERASE ALFA:Plasma cell myeloma:0.38), (TANDOSPIRONE:Plasma cell myeloma:0.01), (TASIMELTEON:Plasma cell myeloma:0.79), (TEDUGLUTIDE:Plasma cell myeloma:1.23), (TEDUGLUTIDE\WATER:Plasma cell myeloma:1.76), (TEGAFURURACIL:Plasma cell myeloma:0.21), (TEGASEROD:Plasma cell myeloma:0.54), (TEGASEROD MALEATE:Plasma cell myeloma:1.63), (TELAPREVIR:Plasma cell myeloma:0.52), (TELITHROMYCIN:Plasma cell myeloma:0.16), (TELOTRISTAT ETHYL:Plasma cell myeloma:1.11), (TEMOCAPRIL:Plasma cell myeloma:0.3), (TEMOCAPRIL HYDROCHLORIDE:Plasma cell myeloma:0.98), (TESAMORELIN:Plasma cell myeloma:0.09), (TETRACAINE:Plasma cell myeloma:0.02), (TETRACAINE HYDROCHLORIDE:Plasma cell myeloma:0.48), (THYMOL:Plasma cell myeloma:0.35), (TIAGABINE HYDROCHLORIDE:Plasma cell myeloma:0.18), (TIARAMIDE:Plasma cell myeloma:0.5), (TIBOLONE:Plasma cell myeloma:1.34), (TIQUIZIUM BROMIDE:Plasma cell myeloma:0.71), (TOCILIZUMAB:Plasma cell myeloma:0.27), (TOLMETIN SODIUM:Plasma cell myeloma:0.3), (TOPIROXOSTAT:Plasma cell myeloma:0.14), (TRANILAST:Plasma cell myeloma:0.37), (TRANYLCYPROMINE SULFATE:Plasma cell myeloma:0.41), (TREPROSTINIL DIOLAMINE:Plasma cell myeloma:1.06), (TREPROSTINIL SODIUM:Plasma cell myeloma:0.25), (TRICHLOROACETIC ACID:Plasma cell myeloma:0.28), (TROGLITAZONE:Plasma cell myeloma:0.38), (TROXIPIDE:Plasma cell myeloma:0.72), (UNDECYLENIC ACID:Plasma cell myeloma:0.05), (URACIL:Plasma cell myeloma:0.48), (UROKINASE:Plasma cell myeloma:0.39), (VALDECOXIB:Plasma cell myeloma:1.96), (VALRUBICIN:Plasma cell myeloma:0.31), (VANDETANIB:Plasma cell myeloma:0.67), (VINBLASTINE SULFATE:Plasma cell myeloma:0.52), (VINDESINE SULFATE:Plasma cell myeloma:0.09), (VINORELBINE TARTRATE:Plasma cell myeloma:1.4), (VITAMIN A PALMITATE:Plasma cell myeloma:0.81), (VORAPAXAR SULFATE:Plasma cell myeloma:0.64), (XYLITOL:Plasma cell myeloma:0.31), (ZALTOPROFEN:Plasma cell myeloma:0.91), (ZICONOTIDE ACETATE:Plasma cell myeloma:1.22), (ZINC CITRATE:Plasma cell myeloma:0.77), (ZIPRASIDONE HYDROCHLORIDE:Plasma cell myeloma:0.14), (ZIV-AFLIBERCEPT:Plasma cell myeloma:0.83), (DILTIAZEM:Plasma cell myeloma in remission:0.06), (UNSPECIFIED INGREDIENT:Plasma cell myeloma in remission:0.74), (ALENDRONIC ACID:Plasma cell myeloma recurrent:0.21), (AMPHETAMINE ASPARTATEAMPHETAMINE SULFATEDEXTROAMPHETAMINE SACCHARATEDEXTROAMPHETAMINE SULFATE:Plasma cell myeloma recurrent:0.21), (ANASTROZOLE:Plasma cell myeloma recurrent:0.47), (ARMODAFINIL:Plasma cell myeloma recurrent:0.1), (BENDAMUSTINE HYDROCHLORIDE:Plasma cell myeloma recurrent:0.06), (BISOPROLOL:Plasma cell myeloma recurrent:0.05), (CANDESARTAN CILEXETILHYDROCHLOROTHIAZIDE:Plasma cell myeloma recurrent:0.36), (CARVEDILOL PHOSPHATE:Plasma cell myeloma recurrent:0.14), (CEFTRIAXONE SODIUM:Plasma cell myeloma recurrent:0.19), (CERTOLIZUMAB PEGOL:Plasma cell myeloma recurrent:0.48), (CLONIDINE:Plasma cell myeloma recurrent:0.55), (DASATINIB:Plasma cell myeloma recurrent:0.39), (DEXLANSOPRAZOLE:Plasma cell myeloma recurrent:0.74), (DOXAZOSIN MESYLATE:Plasma cell myeloma recurrent:0.17), (EDOXABAN TOSYLATE:Plasma cell myeloma recurrent:0.13), (ELETRIPTAN HYDROBROMIDE:Plasma cell myeloma recurrent:0.03), (EVEROLIMUS:Plasma cell myeloma recurrent:0.25), (EVOLOCUMAB:Plasma cell myeloma recurrent:0.17), (FESOTERODINE FUMARATE:Plasma cell myeloma recurrent:0.19), (FLECAINIDE:Plasma cell myeloma recurrent:0.27), (GLICLAZIDE:Plasma cell myeloma recurrent:0.04), (HYDROCHLOROTHIAZIDELOSARTAN POTASSIUM:Plasma cell myeloma recurrent:0.13), (IBRUTINIB:Plasma cell myeloma recurrent:1.35), (IDELALISIB:Plasma cell myeloma recurrent:0.22), (LEDIPASVIRSOFOSBUVIR:Plasma cell myeloma recurrent:0.07), (LERCANIDIPINE:Plasma cell myeloma recurrent:0.19), (LEVALBUTEROL HYDROCHLORIDE:Plasma cell myeloma recurrent:0.25), (LISDEXAMFETAMINE DIMESYLATE:Plasma cell myeloma recurrent:0.15), (METAXALONE:Plasma cell myeloma recurrent:0.36), (METHENAMINE HIPPURATE:Plasma cell myeloma recurrent:0.11), (METHOCARBAMOL:Plasma cell myeloma recurrent:0.27), (METHYLPREDNISOLONE SODIUM SUCCINATE:Plasma cell myeloma recurrent:0.98), (METOLAZONE:Plasma cell myeloma recurrent:0.03), (MOXIFLOXACIN HYDROCHLORIDE:Plasma cell myeloma recurrent:0.23), (NILOTINIB:Plasma cell myeloma recurrent:0.79), (OLANZAPINE:Plasma cell myeloma recurrent:0.23), (OXCARBAZEPINE:Plasma cell myeloma recurrent:0.22), (PENICILLIN:Plasma cell myeloma recurrent:0.01), (PERINDOPRIL:Plasma cell myeloma recurrent:0.01), (PONATINIB:Plasma cell myeloma recurrent:0.08), (PRAMIPEXOLE DIHYDROCHLORIDE:Plasma cell myeloma recurrent:0.36), (PRIMIDONE:Plasma cell myeloma recurrent:0.31), (RANOLAZINE:Plasma cell myeloma recurrent:0.67), (SODIUM OXYBATE:Plasma cell myeloma recurrent:0.2), (SOTALOL:Plasma cell myeloma recurrent:0.42), (TERIFLUNOMIDE:Plasma cell myeloma recurrent:0.24), (VALGANCICLOVIR HYDROCHLORIDE:Plasma cell myeloma recurrent:0.16), (DILTIAZEM:Plasmacytoma:0.1), (DILTIAZEM HYDROCHLORIDE:Platelet aggregation inhibition:0.34), (EVOLOCUMAB:Platelet aggregation inhibition:0.4), (FOLIC ACID:Platelet aggregation inhibition:1.24), (IBRUTINIB:Platelet aggregation inhibition:0.05), (LOVASTATIN:Platelet aggregation inhibition:0.02), (OMEPRAZOLE MAGNESIUM:Platelet aggregation inhibition:0.03), (POTASSIUM:Platelet aggregation inhibition:0.17), (SACUBITRILVALSARTAN:Platelet aggregation inhibition:0.41), (ACETAMINOPHENCODEINE PHOSPHATE:Platelet count decreased:0.22), (ANASTROZOLE:Platelet count decreased:0.2), (BENDAMUSTINE:Platelet count decreased:0.12), (BUPROPION HYDROCHLORIDE:Platelet count decreased:0.6), (CALCITRIOL:Platelet count decreased:0.03), (CEFTRIAXONE SODIUM:Platelet count decreased:0.03), (CERTOLIZUMAB PEGOL:Platelet count decreased:0.16), (CILASTATIN SODIUMIMIPENEM:Platelet count decreased:0.15), (ESZOPICLONE:Platelet count decreased:0.18), (FERROUS SULFATEFERROUS SULFATE, DRIED:Platelet count decreased:0.98), (GLIPIZIDE:Platelet count decreased:0.2), (IMATINIB MESYLATE:Platelet count decreased:0.36), (LEDIPASVIRSOFOSBUVIR:Platelet count decreased:0.15), (LERCANIDIPINE:Platelet count decreased:0.1), (NAPROXEN:Platelet count decreased:0.99), (NEBIVOLOL:Platelet count decreased:0.49), (NITROFURANTOIN:Platelet count decreased:0.02), (PANTOPRAZOLE:Platelet count decreased:0.83), (POLYETHYLENE GLYCOLS:Platelet count decreased:0.25), (RANITIDINE HYDROCHLORIDE:Platelet count decreased:0.56), (SACUBITRILVALSARTAN:Platelet count decreased:0.57), (TIOTROPIUM BROMIDE MONOHYDRATE:Platelet count decreased:0.25), (TRIAMCINOLONE ACETONIDE:Platelet count decreased:0.01), (VALGANCICLOVIR HYDROCHLORIDE:Platelet count decreased:0.18), (ALBUTEROL:Platelet disorder:0.09), (BUPROPION HYDROCHLORIDE:Platelet disorder:0.24), (CERTOLIZUMAB PEGOL:Platelet disorder:0.23), (DALFAMPRIDINE:Platelet disorder:0.07), (DICLOFENAC:Platelet disorder:0.17), (DICLOFENAC SODIUM:Platelet disorder:0.09), (ENALAPRIL:Platelet disorder:0.84), (ENOXAPARIN:Platelet disorder:0.07), (FERROUS SULFATEFERROUS SULFATE, DRIED:Platelet disorder:0.52), (GLIPIZIDE:Platelet disorder:0.12), (INSULIN DETEMIR:Platelet disorder:0.09), (LOVASTATIN:Platelet disorder:0.21), (MELOXICAM:Platelet disorder:0.22), (NAPROXEN:Platelet disorder:0.73), (RANITIDINE HYDROCHLORIDE:Platelet disorder:0.36), (SACUBITRILVALSARTAN:Platelet disorder:0.42), (UNSPECIFIED INGREDIENT:Platelet disorder:1.13), (VERAPAMIL HYDROCHLORIDE:Platelet disorder:0.67), (ALENDRONIC ACID:Pleural effusion:0.21), (ATOVAQUONE:Pleural effusion:0.35), (BICALUTAMIDE:Pleural effusion:0.19), (BLINATUMOMAB:Pleural effusion:0.09), (BUDESONIDE:Pleural effusion:1.02), (CABAZITAXEL:Pleural effusion:0.08), (CANDESARTAN:Pleural effusion:1.34), (CEFUROXIME:Pleural effusion:0.06), (CETUXIMAB:Pleural effusion:1.03), (CHLORAMBUCIL:Pleural effusion:0.13), (CHOLESTYRAMINE:Pleural effusion:0.1), (CLINDAMYCIN:Pleural effusion:0.74), (CLONIDINE:Pleural effusion:0.16), (CLOZAPINE:Pleural effusion:1.02), (CORTISONEHYDROCORTISONE:Pleural effusion:0.66), (CRIZOTINIB:Pleural effusion:1.13), (CYCLOBENZAPRINE HYDROCHLORIDE:Pleural effusion:0), (DABRAFENIB:Pleural effusion:0.33), (DESLORATADINE:Pleural effusion:0.18), (DIGITOXIN:Pleural effusion:0.03), (DRONABINOL:Pleural effusion:0.28), (DUTASTERIDE:Pleural effusion:0.49), (ELOTUZUMAB:Pleural effusion:0.29), (ENALAPRIL MALEATE:Pleural effusion:0.14), (EPLERENONE:Pleural effusion:0.11), (ERGOCALCIFEROL:Pleural effusion:0.31), (ERYTHROPOIETIN:Pleural effusion:0.78), (ESZOPICLONE:Pleural effusion:0.36), (EVOLOCUMAB:Pleural effusion:0.11), (FESOTERODINE FUMARATE:Pleural effusion:0.04), (FLECAINIDE:Pleural effusion:0.37), (FLUTICASONE:Pleural effusion:0.15), (FLUTICASONE FUROATEVILANTEROL TRIFENATATE:Pleural effusion:0.24), (HYDROCHLOROTHIAZIDELISINOPRIL:Pleural effusion:0.66), (HYDROCHLOROTHIAZIDEVALSARTAN:Pleural effusion:0.31), (ILOPROST:Pleural effusion:0.1), (IMIPENEM:Pleural effusion:0.38), (IMIPRAMINE HYDROCHLORIDE:Pleural effusion:0.12), (INDAPAMIDEPERINDOPRIL:Pleural effusion:0.27), (IPRATROPIUM:Pleural effusion:0.39), (IRON:Pleural effusion:0.73), (LABETALOLLABETALOL HYDROCHLORIDE:Pleural effusion:0.08), (LATANOPROST:Pleural effusion:0.24), (LERCANIDIPINE HYDROCHLORIDE:Pleural effusion:0.04), (LEVOLEUCOVORIN CALCIUM:Pleural effusion:0.75), (METHADONE HYDROCHLORIDE:Pleural effusion:0.18), (MYCOPHENOLATE MOFETILMYCOPHENOLATE MOFETIL HYDROCHLORIDE:Pleural effusion:0.39), (NAPROXEN SODIUM:Pleural effusion:0.01), (OLAPARIB:Pleural effusion:0.17), (OXCARBAZEPINE:Pleural effusion:0.3), (PAROXETINE:Pleural effusion:0.3), (PAROXETINEPAROXETINE HYDROCHLORIDE:Pleural effusion:0.04), (PEMETREXED DISODIUM:Pleural effusion:0.7), (POLYETHYLENE GLYCOL 3350:Pleural effusion:0.12), (PRAMIPEXOLE:Pleural effusion:0.11), (PRAVASTATIN:Pleural effusion:0.61), (PRAVASTATIN SODIUM:Pleural effusion:0.61), (PROMETHAZINEPROMETHAZINE HYDROCHLORIDE:Pleural effusion:0.22), (RABEPRAZOLE:Pleural effusion:0.19), (RANOLAZINE:Pleural effusion:0.52), (REGORAFENIB:Pleural effusion:0.65), (SECUKTINUMAB:Pleural effusion:0.35), (SITAGLIPTIN:Pleural effusion:0.1), (SODIUM BICARBONATE:Pleural effusion:0.65), (SOTALOL:Pleural effusion:0.44), (TIMOLOL:Pleural effusion:0.31), (TIOTROPIUM:Pleural effusion:0.2), (UMECLIDINIUM BROMIDEVILANTEROL TRIFENATATE:Pleural effusion:0.03), (VEMURAFENIB:Pleural effusion:0.13), (VERAPAMIL HYDROCHLORIDE:Pleural effusion:0.66), (VITAMIN B:Pleural effusion:0.01), (RIFAMPIN:Pleuritic pain:0.72), (PYRAZINAMIDE:Pneumocystis jirovecii infection:0.61), (ACENOCOUMAROL:Pneumocystis jirovecii pneumonia:0.07), (AMINOSALICYLIC ACID:Pneumocystis jirovecii pneumonia:0.07), (ANASTROZOLE:Pneumocystis jirovecii pneumonia:0.67), (ANIDULAFUNGIN:Pneumocystis jirovecii pneumonia:0.2), (BELATACEPT:Pneumocystis jirovecii pneumonia:0.03), (BEPRIDIL:Pneumocystis jirovecii pneumonia:0.05), (BISACODYL OR DOCUSATE SODIUM:Pneumocystis jirovecii pneumonia:0.18), (BLINATUMOMAB:Pneumocystis jirovecii pneumonia:0.87), (BUDESONIDEFORMOTEROL FUMARATE DIHYDRATE:jirovecii pneumonia:0.14), (BUMETANIDE:Pneumocystis jirovecii pneumonia:0.62), (CALCIUM ACETATE:Pneumocystis jirovecii pneumonia:0.07), (CALCIUM CARBONATECHOLECALCIFEROL:Pneumocystis jirovecii pneumonia:1.45), (CANAKINUMAB:Pneumocystis jirovecii pneumonia:0.12), (CANDESARTAN CILEXETILHYDROCHLOROTHIAZIDE:jirovecii pneumonia:0.78), (CARMUSTINE:Pneumocystis jirovecii pneumonia:0.3), (CEFAZOLIN:Pneumocystis jirovecii pneumonia:0.47), (CEFAZOLIN SODIUM:Pneumocystis jirovecii pneumonia:0.4), (CEFOTAXIMECEFOTAXIME SODIUM:Pneumocystis jirovecii pneumonia:0.2), (CETUXIMAB:Pneumocystis jirovecii pneumonia:0.55), (CHOLESTYRAMINE:Pneumocystis jirovecii pneumonia:0.35), (CLOFARABINE:Pneumocystis jirovecii pneumonia:0.27), (CLOZAPINE:Pneumocystis jirovecii pneumonia:0.03), (DABRAFENIB:Pneumocystis jirovecii pneumonia:0.08), (DACLIZUMAB:Pneumocystis jirovecii pneumonia:0.05), (DANAZOL:Pneumocystis jirovecii pneumonia:0.24), (DASATINIB:Pneumocystis jirovecii pneumonia:0.75), (DEXLANSOPRAZOLE:Pneumocystis jirovecii pneumonia:0.18), (DICLOFENAC:Pneumocystis jirovecii pneumonia:0.31), (DILTIAZEM:Pneumocystis jirovecii pneumonia:0.58), (DULOXETINE:Pneumocystis jirovecii pneumonia:0.01), (ECONAZOLE:Pneumocystis jirovecii pneumonia:0.36), (ELOTUZUMAB:Pneumocystis jirovecii pneumonia:0.45), (ERYTHROMYCIN:Pneumocystis jirovecii pneumonia:0.32), (FERROUS CITRATE:Pneumocystis jirovecii pneumonia:0.21), (FISH OIL:Pneumocystis jirovecii pneumonia:0.29), (FLECAINIDE:Pneumocystis jirovecii pneumonia:0.34), (FLUTICASONE:Pneumocystis jirovecii pneumonia:0.17), (FLUTICASONE FUROATEVILANTEROL TRIFENATATE:Pneumocystis jirovecii pneumonia:0.28), (FOSFOMYCIN:Pneumocystis jirovecii pneumonia:0.31), (FUSIDIC ACID:Pneumocystis jirovecii pneumonia:0.12), (GLIPIZIDE:Pneumocystis jirovecii pneumonia:0.03), (GLYCOPYRRONIUMINDACATEROL:Pneumocystis jirovecii pneumonia:0.23), (IBRITUMOMAB TIUXETAN:Pneumocystis jirovecii pneumonia:1.09), (ILOPROST:Pneumocystis jirovecii pneumonia:0.18), (IMIPRAMINE HYDROCHLORIDE:Pneumocystis jirovecii pneumonia:0.26), (IPRATROPIUM:jirovecii pneumonia:0.38), (IRINOTECAN HYDROCHLORIDE:Pneumocystis jirovecii pneumonia:0.37), (ISOSORBIDE MONONITRATE:Pneumocystis jirovecii pneumonia:0.35), (LABETALOLLABETALOL HYDROCHLORIDE:Pneumocystis jirovecii pneumonia:0.34), (LATANOPROST:Pneumocystis jirovecii pneumonia:0.1), (LEDIPASVIRSOFOSBUVIR:Pneumocystis jirovecii pneumonia:0.22), (LERCANIDIPINE HYDROCHLORIDE:Pneumocystis jirovecii pneumonia:0.29), (LEVALBUTEROL HYDROCHLORIDE:Pneumocystis jirovecii pneumonia:0.1), (LEVOLEUCOVORIN CALCIUM:Pneumocystis jirovecii pneumonia:0.51), (LOVASTATIN:Pneumocystis jirovecii pneumonia:0.03), (MELOXICAM:Pneumocystis jirovecii pneumonia:0.41), (METHADONE HYDROCHLORIDE:Pneumocystis jirovecii pneumonia:0.1), (METHENOLONE ENANTHATE:Pneumocystis jirovecii pneumonia:0.46), (METHIMAZOLE:jirovecii pneumonia:0.15), (METILDIGOXIN:Pneumocystis jirovecii pneumonia:0.03), (MINOCYCLINEMINOCYCLINE HYDROCHLORIDE:jirovecii pneumonia:0.33), (NELFINAVIR:Pneumocystis jirovecii pneumonia:0), (NICARDIPINE:Pneumocystis jirovecii pneumonia:0.02), (NITROFURANTOIN:Pneumocystis jirovecii pneumonia:0.36), (OLODATEROL HYDROCHLORIDETIOTROPIUM BROMIDE MONOHYDRATE:Pneumocystis jirovecii pneumonia:0.03), (OXCARBAZEPINE:jirovecii pneumonia:0.11), (PANTOPRAZOLE MAGNESIUM:Pneumocystis jirovecii pneumonia:0.29), (PEMETREXED:Pneumocystis jirovecii pneumonia:0.26), (PENTOSTATIN:Pneumocystis jirovecii pneumonia:0.04), (PILSICAINIDE HYDROCHLORIDE:Pneumocystis jirovecii pneumonia:0.14), (PIMOBENDAN:jirovecii pneumonia:0.03), (POTASSIUM CITRATETRISODIUM CITRATE DIHYDRATE:Pneumocystis jirovecii pneumonia:0.14), (PRANLUKAST:Pneumocystis jirovecii pneumonia:0.3), (PROMETHAZINE HYDROCHLORIDE:Pneumocystis jirovecii pneumonia:0.03), (PROMETHAZINEPROMETHAZINE HYDROCHLORIDE:jirovecii pneumonia:0.25), (PYRIDOXAL:Pneumocystis jirovecii pneumonia:0.18), (REGORAFENIB:Pneumocystis jirovecii pneumonia:0.07), (ROFLUMILAST:Pneumocystis jirovecii pneumonia:0.18), (SACUBITRILVALSARTAN:Pneumocystis jirovecii pneumonia:0.3), (SECUKINUMAB:Pneumocystis jirovecii pneumonia:0.78), (TIGECYCLINE:Pneumocystis jirovecii pneumonia:0.38), (TINZAPARIN SODIUM:Pneumocystis jirovecii pneumonia:0.38), (TIOTROPIUM:Pneumocystis jirovecii pneumonia:0.13), (TOPIRAMATE:Pneumocystis jirovecii pneumonia:0.26), (TRAMETINIB:Pneumocystis jirovecii pneumonia:0.08), (VENLAFAXINE HYDROCHLORIDE:Pneumocystis jirovecii pneumonia:0.44), (VINDESINE SULFATE:Pneumocystis jirovecii pneumonia:0.08), (ABOBOTULINUMTOXINA:Pneumonia:0.28), (ACECLOFENAC:Pneumoni a:0.11), (ACETAMINOPHENAPRONALIDECAFFEINEPROPYPHENAZONE:Pneumonia:00.22), (ACETAMINOPHEN\CAFFEINE:Pneumonia:0.55), (ACETAMINOPHEN\CAFFEINEOP IUM:Pneumonia:0.85), (ACETAMINOPHENDIPHENHYDRAMINE:Pneumonia:0.57), (ADEN INE:Pneumonia:0.5), (ADENOSINE TRIPHOSPHATE:Pneumonia:0.52), (AGALSIDASE BETA:Pneumonia:1.06), (ALBIGLUTIDE:Pneumonia:0.09), (ALBUTEROLGUAIFENESIN:Pn eumonia:0.52), (AMILORIDE HYDROCHLORIDEHYDROCHLOROTHIAZIDE:Pneumonia:1.29), (AMLODIPINE BESYLATECANDESARTAN CILEXETIL:Pneumonia:0.5), (AMLODIPINE BESYLATEIRBESARTAN:Pneumonia:0.87), (AMLODIPINEPERINDOPRIL:Pneumonia:0.73), (AMMONIUM LACTATE:Pneumonia:1.11), (AMOXAPINE:Pneumonia:1.13), (AMPHOTERICIN BDIMYRISTOYLPHOSPHATIDYLCHOLINE, DL-DIMYRISTOYLPHOSPHATIDYLGLYCEROL, DL-:Pneumonia:0.63), (AMSACRINE:Pneumonia:0.04), (ANAGLIPTIN:Pneumonia:0.16), (ANA GRELIDE:Pneumonia:0.25), (ANAGRELIDE HYDROCHLORIDE:Pneumonia:0.34), (ANTIHISTAMINES NOS:Pneumonia:0.96), (ASCORBIC ACIDBIOTINCYANOCOBALAMINFOLIC ACIDNIACINPANTOTHENIC ACIDPYRIDOXINE\RIBOFLAVINTHIAMINE:Pneumonia:0.22), (ASCORBIC ACIDVITAMIN B COMPLEX:Pneumonia:0.03), (ASPARAGINASE:Pneumonia:1.22), (ASPIRINCLOPIDOGREL: Pneumonia:0.53), (ASPIRINDIHYDROXYALUMINUM AMINOACETATE ANHYDROUSMAGNESIUM CARBONATE:Pneumonia:0.28), (ATAZANAVIR:Pneumonia:0.32), (ATRACURIUM:Pneumon ia:0.58), (AXITINIB:Pneumonia:0.73), (AZITHROMYCIN OR METHOTREXATE SODIUM:Pneumonia:0.61), (BALSALAZIDE DISODIUM:Pneumonia:0.48), (BAMIFYLLINE HYDROCHLORIDE:Pneumonia:0.1), (BELATACEPT:Pneumonia:0.44), (BENIDIPINE:Pneum onia:0.1), (BENZTROPINE:Pneumonia:0.52), (BETAMETHASONE DIPROPIONATECALCIPOTRIENECALCIPOTRIENE HYDRATE:Pneumonia:0.45), (BETAMETHASONE SODIUM PHOSPHATENEOMYCIN SULFATE:Pneumonia:0.06), (BETHANECHOL CHLORIDE:Pneumonia:0.25), (BICARBONATE ION:Pneumonia:0.5), (BILASTINE:Pneumonia:1.23), (BLESELUMAB:Pneumonia:0.18), (BOSU TINIB:Pneumonia:0.12), (BOTULISM ANTITOXIN:Pneumonia:0.35), (BRIVARACETAM:Pneumonia:0.14), (BUCILLAMINE:Pneum onia:2), (BUPIVACAINE HYDROCHLORIDE:Pneumonia:0.33), (CABAZITAXEL:Pneumonia:0.91), (CALCIUM CARBONATEERGOCALCIFEROLRETINOL:Pneumonia:0.79), (CALCIUM\MAGNESIUM ZINC:Pneumonia:0.28), (CAMPHORDIPHENHYDRAMINE\ZINC OXIDE:Pneumonia:0.03), (CANAGLIFLOZINMETFORMIN HYDROCHLORIDE:Pneumonia:0.95), (CAPSAICIN:Pneumonia:0.27), (CARBIDOPA:Pneumo nia:0.35), (CARBOMER:Pneumonia:0.18), (CARTEOLOL:Pneumonia:0.02), (CARTEOLOL HYDROCHLORIDE:Pneumonia:0.12), (CASANTHRANOLDOCUSATE SODIUM:Pneumonia:0.76), (CEFPIROME:Pneumonia:0.16), (CEFPIROME SULFATE:Pneumonia:1.24), (CEFTERAM PIVOXIL:Pneumonia:0.08), (CERTOPARIN SODIUM:Pneumonia:0.65), (CHLORDIAZEPOXIDE HYDROCHLORIDECLIDINIUM BROMIDE:Pneumonia:0.24), (CHONDROITIN SULFATE (CHICKEN):Pneumonia:0.89), (CIFENLINE:Pneumonia:0.52), (CISAPRIDE:Pneumonia:0.02), (CLENBUTEROL HYDROCHLORIDE:Pneumonia:0.68), (CLODRONATE DISODIUM:Pneumonia:0.06), (CLOXAZOLAM:Pneumonia:0.06), (COD LIVER OIL:Pneumonia:0.71), (COLCHICINEDICYCLOMINE HYDROCHLORIDE:Pneumonia:0.41), (CROTALIDAE POLYVALENT IMMUNE FAB (OVINE):Pneumonia:0.08), (CYCLOPHOSPHAMIDECYTARABINEDEXAMETHASONED OXORUBICINMETHOTREXATEVINCRISTINE:Pneumonia:0.04), (DABRAFENIB MESYLATE:Pneumonia:0.1), (DEFERIPRONE:Pneumonia:0.19), (DEFIBROTIDE (BOVINE):Pneumonia:0.34), (DEOXYRIBONUCLIEC ACID:Pneumonia:0.31), (DEQUALINIUM CHLORIDE:Pneumonia:0.22), (DESVENLAFAXINE:Pneumonia:0.1), (DEXTRAN:Pneumonia: 0.09), (DEXTROAMPHETAMINE SULFATE:Pneumonia:0.14), (DISOPYRAMIDE:Pneumonia:0.52), (DOLASETRON MESYLATE:Pneumonia:0.51), (DOLUTEGRAVIR:Pneumonia:0.73), (DORZOLAMIDE HYDROCHLORIDE:Pneumonia:0.88), (DOXEPIN HYDROCHLORIDE:Pneumonia:0.88), (DOXYLAMINE SUCCINATE:Pneumonia:0.6), (DULAGLUTIDE:Pneumonia:0.18), (ECABET:Pneumonia:0.07), (ELTROMBOPAG OLAMINE:Pneumonia:0.54), (EMTRICITABINERILPIVIRINETENOFOVIR DISOPROXIL FUMARATE:Pneumonia:0.35), (ENALAPRIL MALEATEHYDROCHLOROTHIAZIDE:Pneumonia:0.72), (EPHEDRINE:Pneumonia:0.26), (EPHEDRINE HYDROCHLORIDE:Pneumonia:0.64), (ERIBULIN:Pneumonia:0.09), (ERIBULIN MESYLATE:Pneumonia:0.31), (ETHACRYNIC ACID:Pneumonia:0.32), (ETHINYL ESTRADIOLNORGESTREL:Pneumonia:0.34), (ETHIONAMIDE:Pneumonia:0.46), (ETOPOSIDE PHOSPHATE:Pneumonia:1.02), (ETRETINATE:Pneumonia:1.83), (EVOLOCUMAB:Pneumoni a:1.35), (FENOFIBRIC ACID:Pneumonia:0.83), (FERRIC HYDROXIDE:Pneumonia:0.13), (FERRIC PYROPHOSPHATE:Pneumonia:0.55), (FERROUS SULFATEGLYCINE:Pneumonia:1.25), (FIBRINOGEN HUMAN:Pneumonia:0.24), (FLAVIN ADENINE DINUCLEOTIDE:Pneumonia:1.11), (FLECAINIDE ACETATE:Pneumonia:0.13), (FLOMOXEF:Pneumonia:0.28), (FLUOCINOLONE ACETONIDE:Pneumonia:1.38), (FLUTAMIDE:Pneumonia:0.13), (FOLATE SODIUM:Pneumonia:0.74), (FOSAPREPITANT:Pneumonia:0.11), (FOSFOMYCIN SODIUM:Pneumonia:0.14), (FURSULTIAMINE:Pneumonia:0.5), (GLIPIZIDEMETFORMIN HYDROCHLORIDE:Pneumonia:0.08), (GLYCOPYRRONIUM:Pneumonia:1.96), (GOSERELI N ACETATE:Pneumonia:0.29), (GUANABENZ ACETATE:Pneumonia:0.56), (GUANFACINE:Pneumonia:0.52), (HUMAN BLOOD CELLS:Pneumonia:0.05), (HYDROCHLOROTHIAZIDERAMIPRIL:Pneumonia:0.51), (HYDR OCHLOROTHIAZIDESPIRONOLACTONE:Pneumonia:0.08), (HYDROCODONE BITARTRATEIBUPROFEN:Pneumonia:0.3), (HYDROCORTISONE HEMISUCCINATE:Pneumonia:0.3), (HYDROCORTISONE PROBUTATE:Pneumonia:0.19), (HYOSCYAMINE:Pneumonia:1.04), (IBRITUMOMAB TIUXETAN:Pneumonia:1.87), (IFENPRODIL:Pneumonia:0.23), (ILOPROST:Pneumonia:1.76), (IMIGLUCERASE:Pneumonia:0.41), (IMIPRAMINE:Pneumonia:0.37), (INDAPAMIDEPERIN DOPRIL:Pneumonia:2.59), (INFLIXIMAB-DYYB:Pneumonia:0.67), (INTERFERON ALFA-2A:Pneumonia:1), (INTERFERON ALFA-2B:Pneumonia:0.23), (INTERFERON GAMMA-1B:Pneumonia:1.57), (IRONPOLYSACCHARIDES:Pneumonia:0.11), (KALLIDINOGENASE: Pneumonia:0.21), (KETOTIFEN:Pneumonia:0.11), (LACTASE:Pneumonia:0.94), (LACTOBACI LLUS RHAMNOSUS:Pneumonia:1.54), (LAPATINIB DITOSYLATE:Pneumonia:0.41), (LECITHIN:Pneumonia:0.23), (LENVATINIB:Pneumonia:0.26), (LEVALBUTEROL TARTRATE:Pneumonia:0.04), (LEVOCABASTINE HYDROCHLORIDE:Pneumonia:0.68), (LOMITAPIDE MESYLATE:Pneumonia:0.49), (LOMUSTINE:Pneumonia:0.55), (LORNOXICAM:Pneumonia:00.39), (LUMACAFTOR:Pneumonia:1), (LURASIDONE HYDROCHLORIDE:Pneumonia:0.29), (MAGNESIUM PIDOLATE:Pneumonia:0.04), (MEPENZOLATE BROMIDE:Pneumonia:0.16), (METFORMIN HYDROCHLORIDESAXAGLIPTIN:Pneumonia:0.02), (METFORMIN HYDROCHLORIDEVILDAGLIPTIN:Pneumonia:1.04), (METFORMIN PAMOATE:Pneumonia:0.56), (METHENAMINE HIPPURATE:Pneumonia:1.92), (METHENOLONE ACETATE:Pneumonia:0.22), (METHYLENE BLUE:Pneumonia:0.3), (METHYLPHENIDATE:Pneumonia:0.07), (METHYLPREDNISOLON E HEMISUCCINATE:Pneumonia:0.22), (MEXILETINE:Pneumonia:0.08), (MICONAZOLE NITRATE:Pneumonia:0.63), (MILRINONE:Pneumonia:0.48), (MITIGLINIDE CALCIUM HYDRATEVOGLIBOSE:Pneumonia:0.2), (MITOXANTRONE HYDROCHLORIDE:Pneumonia:0.96), (MOEXIPRIL HYDROCHLORIDE:Pneumonia:0.08), (MOGAMULIZUMAB:Pneumonia:0.47), (NADIFLOX ACIN:Pneumonia:0.09), (NALTREXONE:Pneumonia:0.2), (NUSINERSEN:Pneumonia:0.03), (O LMESARTAN:Pneumonia:0.8), (ORPHENADRINE:Pneumonia:0.94), (OSPEMIFENE:Pneumo nia:0.02), (OXYBUTYNINOXYBUTYNIN CHLORIDE:Pneumonia:0.04), (PANCRELIPASEPANCRELIPASE AMYLASEPANCRELIPASE LIPASEPANCRELIPASE PROTEASE:Pneumonia:0.39), (PARATHYROID HORMONE:Pneumonia:0.09), (PARICALCITOL:Pneumonia:0.9), (PASIREOTIDE:Pneumonia: 0.69), (PATIROMER:Pneumonia:0.35), (PEGINTERFERON BETA-1A:Pneumonia:0.94), (PENTOSTATIN:Pneumonia:1.44), (PHENOTHIAZINE:Pneumonia:0.06), (PILOCARPINE:Pneumonia:0.72), (PIRENZEPINE:Pneumonia:0.21), (PIRITRAMIDE:Pneumo nia:0.68), (PIROXICAM:Pneumonia:0.92), (PLERIXAFOR:Pneumonia:0.13), (POLYSTYRENE: Pneumonia:0.31), (PORFIMER SODIUM:Pneumonia:0.99), (POTASSIUM IODIDE:Pneumonia:0.78), (PRASUGREL:Pneumonia:0.1), (PRAZOSIN HYDROCHLORIDE:Pneumonia:0.78), (PREDNISOLONE VALERATE ACETATE:Pneumonia:0.12), (PYRIDOXAL PHOSPHATE:Pneumonia:0.42), (PYRIMETHAMINE:Pneumonia:0.67), (QUAZEPAM:Pneumo nia:0.19), (QUINACRINE:Pneumonia:0.66), (RADIATION THERAPY:Pneumonia:1.22), (RANIMUSTINE:Pneumonia:0.76), (REGORAFENIB:Pneumonia: 0.75), (RHUBARB:Pneumonia:0.01), (RILPIVIRINE HYDROCHLORIDE:Pneumonia:0.62), (RISEDRONIC ACID:Pneumonia:0.04), (RIVASTIGMINE TARTRATE:Pneumonia:1.75), (RIZATRIPTAN:Pneumonia:0.3), (ROPIVACAINE:Pneumonia: 0.35), (SARPOGRELATE HYDROCHLORIDE:Pneumonia:0.87), (SCOPOLAMINE HYDROBROMIDE:Pneumonia:0.06), (SENNOSIDE B:Pneumonia:0.12), (SEVELAMER:Pneumonia:0.41), (SILICON DIOXIDE:Pneumonia:0.3), (SODIUM BICARBONATE\SODIUM CHLORIDE:Pneumonia:0.13), (SODIUM CITRATE:Pneumonia:0.8), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Pneumonia:0.02), (SOFALCONE:Pneumonia:0.04), ( FAECALIS:Pneumonia:0.3), (STREPTOMYCIN SULFATE:Pneumonia:0.82), (SULFAMERAZINETRIMETHOPRIM:Pneumonia:0.99), (SUPL ATAST TOSILATE:Pneumonia:1.48), (TAFAMIDIS MEGLUMINE:Pneumonia:0.28), (TEMOCAPRIL HYDROCHLORIDE:Pneumonia:0.98), (TEMSIROLIMUS:Pneumonia:0.16), (TILIDINE:Pneum onia:0.15), (TIMOLOL MALEATETRAVOPROST:Pneumonia:0.3), (TIPIRACIL HYDROCHLORIDETRIFLURIDINE:Pneumonia:0.15), (TIQUIZIUM BROMIDE:Pneumonia:0.11), (TISAGENLECLEUCEL:Pneumonia:0.43), (TRAMETINIB DIMETHYL SULFOXIDE:Pneumonia:0.09), (TREPROSTINIL DIOLAMINE:Pneumonia:0.07), (TROXIPIDE:Pneumonia:0.29), (TULOBUTEROL HYDROCHLORIDE:Pneumonia:0.15), (UROKINASE:Pneumonia:0.74), (VADASTUXIMAB TALIRINE:Pneumonia:0.13), (VONOPRAZAN:Pneumonia:0.35), (ZAFIRLUKAST:Pneumonia: 1.12), (ZALTOPROFEN:Pneumonia:0.64), (ZICONOTIDE ACETATE:Pneumonia:0.11), (ZIPRASIDONE HYDROCHLORIDEZIPRASIDONE MESYLATE:Pneumonia:0.22), (ZIV-AFLIBERCEPT:Pneumonia:0.24), (ACENOCOUMAROL:Pneumonia aspiration:0.1), (ALBUTEROL SULFATEIPRATROPIUM BROMIDE:Pneumonia aspiration:0.18), (ALCOHOL:Pneumonia aspiration:0.28), (AMISULPRIDE:Pneumonia aspiration:0.25), (AMITRIPTYLINE:Pneumonia aspiration:0.24), (BUMETANIDE:Pneumonia aspiration:0.4), (BUPRENORPHINE HYDROCHLORIDENALOXONE HYDROCHLORIDE:Pneumonia aspiration:0.29), (BUPROPION HYDROCHLORIDE:Pneumonia aspiration:0.78), (CALCIUM CARBONATECHOLECALCIFEROL:Pneumonia aspiration:0.11), (CANDESARTAN CILEXETILHYDROCHLOROTHIAZIDE:Pneumonia aspiration:0.14), (CASPOFUNGIN:Pneumonia aspiration:0.49), (CEPHALEXIN:Pneumonia aspiration:0.64), (CETIRIZINE HYDROCHLORIDE:Pneumonia aspiration:0.24), (CISATRACURIUM:Pneumonia aspiration:0.36), (COLCHICINE:Pneumonia aspiration:0.03), (DABIGATRAN:Pneumonia aspiration:0.02), (DEXLANSOPRAZOLE:Pneumonia aspiration:0.03), (DILTIAZEM HYDROCHLORIDE:Pneumonia aspiration:0.43), (EDOXABAN TOSYLATE:Pneumonia aspiration:0.08), (ESMOLOL HYDROCHLORIDE:Pneumonia aspiration:0.3), (FENOFIBRATE:Pneumonia aspiration:0.06), (FLECAINIDE:Pneumonia aspiration:0.33), (HYDRALAZINE HYDROCHLORIDE:Pneumonia aspiration:0.07), (IBRUTINIB:Pneumonia aspiration:0.34), (INDAPAMIDEPERINDOPRIL:Pneumonia aspiration:0.36), (IRON:Pneumonia aspiration:0.29), (ISOFLURANE:Pneumonia aspiration:0.21), (ISOSORBIDE MONONITRATE:Pneumonia aspiration:0.05), (LABETALOLLABETALOL HYDROCHLORIDE:Pneumonia aspiration:0.53), (LEDIPASVIRSOFOSBUVIR:Pneumonia aspiration:0.33), (LOVASTATIN:Pneumonia aspiration:0.08), (METOPROLOL TARTRATE:Pneumonia aspiration:0.18), (MOXIFLOXACIN HYDROCHLORIDE:Pneumonia aspiration:0.47), (NAPROXEN:Pneumonia aspiration:0.34), (NEBIVOLOL:Pneumonia aspiration:0.43), (NEOSTIGMINE:Pneumonia aspiration:0.36), (NIFEDIPINE:Pneumonia aspiration:0.37), (PAROXETINEPAROXETINE HYDROCHLORIDE:Pneumonia aspiration:0.07), (POSACONAZOLE:Pneumonia aspiration:0.29), (PYRAZINAMIDE:Pneumonia aspiration:0.6), (SITAGLIPTIN:Pneumonia aspiration:0.03), (SOTALOL:Pneumonia aspiration:0.35), (TERBUTALINE:Pneumonia aspiration:0.06), (TERIFLUNOMIDE:Pneumonia aspiration:0.27), (THIAMYLAL SODIUM:Pneumonia aspiration:0.27), (TINZAPARIN SODIUM:Pneumonia aspiration:0.11), (TIZANIDINE HYDROCHLORIDE:Pneumonia aspiration:0.06), (AMIODARONE HYDROCHLORIDE:Pneumonitis:0.38), (AMITRIPTYLINE:Pneumonitis:0.17), (BENDAMUS TINE HYDROCHLORIDE:Pneumonitis:0.1), (BICALUTAMIDE:Pneumonitis:0.04), (CANDESARTA N CILEXETIL:Pneumonitis:0.21), (CANDESARTAN CILEXETILHYDROCHLOROTHIAZIDE:Pneumonitis:0.2), (CARFILZOMIB:Pneumonitis:0.13), (CERTOLIZUMAB PEGOL:Pneumonitis:0.55), (CICLESONIDE:Pneumonitis:0.15), (DASATINIB:Pneumonitis:0.71), (DEXLANSOPRAZOLE:Pneumonitis:0.49), (ENALAPRIL:Pneumonitis:1.38), (EPIRUBICIN: Pneumonitis:0.31), (EVOLOCUMAB:Pneumonitis:0.39), (FLECAINIDE:Pneumonitis:0.08), (FO RMOTEROL FUMARATE\MOMETASONE FUROATE:Pneumonitis:0.12), (FULVESTRANT:Pneumonitis:0.2), (HEPARIN SODIUM:Pneumonitis:0.32), (HYDROCHLOROTHIAZIDELISINOPRIL:Pneumonitis:0.22), (HYDROMORPHONE:Pneumonitis:0.13), (INDAPAMIDEPERINDOPRIL:Pneumonitis:0.24), (INFLUENZA VIRUS VACCINE:Pneumonitis:0.06), (IPRATROPIUM:Pneumonitis:0.19), (IRINOTECAN HYDROCHLORIDE:Pneumonitis:0.09), (ISOSORBIDE MONONITRATE:Pneumonitis:0.29), (LEDIPASVIRSOFOSBUVIR:Pneumonitis:0.08), (LOVA STATIN:Pneumonitis:0.3), (MAGNESIUM:Pneumonitis:0.43), (MICAFUNGIN:Pneumonitis:0.22), (NAPROXEN:Pneumonitis:1.04), (OXCARBAZEPINE:Pneumonitis:0.25), (PANTOPRAZOL E MAGNESIUM:Pneumonitis:0.56), (POLYETHYLENE GLYCOLS:Pneumonitis:0.59), (PRAVASTATIN SODIUM:Pneumonitis:0.29), (SACUBITRILVALSARTAN:Pneumonitis:0.59), (SECUKINUM AB:Pneumonitis:0.21), (TOCILIZUMAB:Pneumonitis:0.31), (TOFACITINIB CITRATE:Pneumonitis:0.22), (VANCOMYCIN HYDROCHLORIDE:Pneumonitis:0.25), (ZOLPIDEMZOLPIDEM TARTRATE:Pneumonitis:0.18), (ACARBOSE:Pollakiuria:0.68), (AGALSIDASE BETA:Pollakiuria:0.15), (ALBIGLUTIDE:Pollakiuria:0.13), (ALENDRONIC ACID:Pollakiuria:0.65), (AMISULPRIDE:Pollakiuria:0.32), (APREPITANT:Pollakiuria:0.41), (A RMODAFINIL:Pollakiuria:1.38), (AZITHROMYCIN DIHYDRATE:Pollakiuria:0.49), (BUDESONIDEFORMOTEROL FUMARATE:Pollakiuria:0.44), (BUPRENORPHINE HYDROCHLORIDE:Pollakiuria:0.25), (BUPRENORPHINE HYDROCHLORIDENALOXONE HYDROCHLORIDE:Pollakiuria:0.22), (BUTYLSCOPOLAMINE BROMIDE:Pollakiuria:0.2), (CALCITONIN SALMON:Pollakiuria:0.18), (CALCIUM ACETATE:Pollakiuria:0.74), (CANAGLIFLOZINMETFORMIN HYDROCHLORIDE:Pollakiuria:0.67), (CANAKINUMAB:Pollakiuria:0.21), (CAPTOPRIL:Poll akiuria:0.61), (CARISOPRODOL:Pollakiuria:0.63), (CARVEDILOL PHOSPHATE:Pollakiuria:1.21), (CERTOLIZUMAB PEGOL:Pollakiuria:1.71), (CHONDROITIN SULFATE (BOVINE)GLUCOSAMINE HYDROCHLORIDE:Pollakiuria:0.29), (CIMETIDINE:Pollakiuria:0.87), (CLINDAMYCIN:Poll akiuria:0.66), (COLESEVELAM HYDROCHLORIDE:Pollakiuria:0.38), (CORTICOTROPIN:Pollakiuria:0.36), (DACLIZUMAB: Pollakiuria:0.55), (DALTEPARIN:Pollakiuria:0.03), (DALTEPARIN SODIUM:Pollakiuria:0.16), (DANAZOL:Pollakiuria:0.43), (DESONIDE:Pollakiuria:0.11), (DEX AMETHASONE ACETATE:Pollakiuria:0.15), (EMPAGLIFLOZINLINAGLIPTIN:Pollakiuria:0.44), (EPIRUBIC IN:Pollakiuria:0.06), (EVOLOCUMAB:Pollakiuria:1.71), (FERROUS FUMARATE:Pollakiuria:0.09), (FEXOFENADINEPSEUDOEPHEDRINE:Pollakiuria:0.33), (F LUDROCORTISONE ACETATE:Pollakiuria:0.51), (FLUTICASONE FUROATEVILANTEROL TRIFENATATE:Pollakiuria:1.21), (FLUVASTATIN SODIUM:Pollakiuria:0.04), (FULVESTRANT:Pollakiuria:0.52), (FUSIDIC ACID:Pollakiuria:0.18), (GIMERACILOTERACILTEGAFUR:Pollakiuria:0.32), (HERBALST URMERIC:Pollakiuria:0.42), (HYDROCHLOROTHIAZIDEIRBESARTAN:Pollakiuria:0.78), (HYDROCORTISONE ACETATE:Pollakiuria:0), (HYDROXYZINE PAMOATE:Pollakiuria:1.51), (ILOPROST:Pollakiuria:0.39), (IMATINIB MESYLATE:Pollakiuria:0.98), (INDAPAMIDEPERINDOPRIL:Pollakiuria:0.83), (INSULIN DEGLUDEC:Pollakiuria:0.43), (IRINOTECAN:Pollakiuria:0.79), (KETOROLACKETOROLA C TROMETHAMINE:Pollakiuria:0.03), (RHAMNOSUS:Pollakiuria:0.06), (LAPATINIB DITOSYLATE:Pollakiuria:0.17), (LERCANIDIPINE HYDROCHLORIDE:Pollakiuria:0.69), (LETROZOLE:Pollakiuria:0.81), (LIOTHYRONINE SODIUM:Pollakiuria:0.43), (LISDEXAMFETAMINE DIMESYLATE:Pollakiuria:0.71), (LOMITAPIDE MESYLATE:Pollakiuria:0.42), (METFORMIN HYDROCHLORIDEVILDAGLIPTIN:Pollakiuria:0.21), (METHADONE HYDROCHLORIDE:Pollakiuria:0.59), (METHENAMINE HIPPURATE:Pollakiuria:0.61), (METHIMAZOLE:Pollakiuria:0.44), (METHYLPREDNISOLO NE ACETATE:Pollakiuria:0.84), (MOXIFLOXACIN:Pollakiuria:0.04), (MOXIFLOXACIN HYDROCHLORIDE:Pollakiuria:1.16), (NADOLOL:Pollakiuria:0.69), (NALOXONE HYDROCHLORIDEOXYCODONE HYDROCHLORIDE:Pollakiuria:0.78), (NALTREXONE:Pollakiuria:0.15), (NILOTINIB:Pollaki uria:1.6), (NIRAPARIB:Pollakiuria:0.27), (NORETHINDRONE:Pollakiuria:0.13), (NORTRIPTY LINE HYDROCHLORIDE:Pollakiuria:0.16), (OLODATEROL HYDROCHLORIDETIOTROPIUM BROMIDE MONOHYDRATE:Pollakiuria:0.74), (OXAZEPAM:Pollakiuria:0.48), (PANTOPRAZOLE MAGNESIUM:Pollakiuria:1.28), (PATIROMER:Pollakiuria:0.16), (PAZOPANIB HYDROCHLORIDE:Pollakiuria:1.14), (PEGFILGRASTIM:Pollakiuria:0.22), (PEMETREXED: Pollakiuria:0.07), (PHENYTOIN SODIUM:Pollakiuria:0.1), (PILSICAINIDE HYDROCHLORIDE:Pollakiuria:0.06), (PIMECROLIMUS:Pollakiuria:0.15), (PIRFENIDONE:P ollakiuria:0.1), (PONATINIB:Pollakiuria:0.62), (PROGESTERONE:Pollakiuria:0.36), (PYRIDO XINE:Pollakiuria:0.24), (REGORAFENIB:Pollakiuria:0.48), (SALMETEROL XINAFOATE:Pollakiuria:0.06), (SCOPOLAMINE:Pollakiuria:0.81), (SEVELAMER CARBONATE:Pollakiuria:0.06), (SULFAMETHOXAZOLE:Pollakiuria:0.45), (TERBINAFINE HYDROCHLORIDE:Pollakiuria:0.52), (TERBUTALINE SULFATE:Pollakiuria:0.56), (THALIDOMIDE:Pollakiuria:0.69), (TIMOLOL MALEATE:Pollakiuria:0.25), (TOCOPHEROL:Pollakiuria:0.09), (TORSEMIDE:Pollakiuria:1.31), (UMECLIDINIUM BROMIDE:Pollakiuria:0.02), (UMECLIDINIUM BROMIDEVILANTEROL TRIFENATATE:Pollakiuria:0.71), (VALGANCICLOVIR HYDROCHLORIDE:Pollakiuria:0.29), (ZAFIRLUKAST:Pollakiuria:0.02), (AMPHETAMINE ASPARTATEAMPHETAMINE SULFATEDEXTROAMPHETAMINE SACCHARATEDEXTROAMPHETAMINE SULFATE:Polyarthritis:0.43), (ANASTROZOLE:Polyarthritis:0.8), (BISACODYL OR DOCUSATE SODIUM:Polyarthritis:0.11), (BUMETANIDE:Polyarthritis:0.75), (CANDESARTAN CILEXETIL:Polyarthritis:0.56), (CIPROFLOXACIN HYDROCHLORIDE:Polyarthritis:0.36), (CLINDAMYCINCLINDAMYCIN PHOSPHATE:Polyarthritis:0.54), (CLOTRIMAZOLE:Polyarthritis:0.11), (DASATINIB:Polyarth ritis:0.28), (DEVICE:Polyarthritis:0.17), (DIVALPROEX SODIUM:Polyarthritis:0.03), (ELETRIPTAN HYDROBROMIDE:Polyarthritis:0.19), (ENOXAPARIN:Polyarthritis:0.55), (EVOLOCUMAB:P olyarthritis:0.8), (FERROUS SULFATE:Polyarthritis:0.63), (GOLD:Polyarthritis:0.01), (HYDROCHLOROTHIAZIDELOSA RTAN POTASSIUM:Polyarthritis:0), (IBRUTINIB:Polyarthritis:0.86), (INDAPAMIDE:Polyarthritis:0.09), (ISOSORBIDE:Polyarthritis:0.06), (ISOSORBIDE MONONITRATE:Polyarthritis:0.54), (LERCANIDIPINE HYDROCHLORIDE:Polyarthritis:0.43), (METHADONE HYDROCHLORIDE:Polyarthritis:0.06), (METHYLPHENIDATE HYDROCHLORIDE:Polyarthritis:0), (METRONIDAZOLEMETRONIDAZOLE HYDROCHLORIDE:Polyarthritis:0.17), (MOXIFLOXACIN HYDROCHLORIDE:Polyarthritis:0), (OLANZAPINE:Polyarthritis:0.13), (OXCARBAZEPINE: Polyarthritis:0.05), (PANTOPRAZOLE MAGNESIUM:Polyarthritis:0.38), (PAROXETINE:Polyarthritis:0.16), (PAROXETINEPAROX ETINE HYDROCHLORIDE:Polyarthritis:0.28), (PRIMIDONE:Polyarthritis:0.17), (PROMETHAZINE PROMETHAZINE HYDROCHLORIDE:Polyarthritis:0.33), (PYRAZINAMIDE:Polyarthritis:0.65), (RANOLAZIN E:Polyarthritis:0.68), (SUMATRIPTANSUMATRIPTAN SUCCINATE:Polyarthritis:0.06), (TAMSULOSIN HYDROCHLORIDE:Polyarthritis:0.23), (TERIFLUNOMIDE:Polyarthritis:0.08), (VARENICLI NE TARTRATE:Polyarthritis:0.11), (VERAPAMIL HYDROCHLORIDE:Polyarthritis:0.82), (ACLIDINIUM BROMIDE:Polycystic ovaries:0.34), (ALLOPURINOL:Polycystic ovaries:0.01), (AMITRIPTYLINE HYDROCHLORIDE:Polycystic ovaries:0.03), (ATROPINE SULFATEDIPHENOXYLATE HYDROCHLORIDE:Polycystic ovaries:0.3), (BISACODYL OR DOCUSATE SODIUM:Polycystic ovaries:0.1), (BUMETANIDE:Polycystic ovaries:0.18), (BUPRENORPHINE HYDROCHLORIDE:Polycystic ovaries:0.3), (CALCIUM CARBONATE:Polycystic ovaries:0.1), (CALCIUM CITRATE:Polycystic ovaries:0.09), (CARVEDILOL PHOSPHATE:Polycystic ovaries:0.17), (CHOLESTYRAMINE:Polycystic ovaries:1.15), (CLINDAMYCIN:Polycystic ovaries:0.16), (CLONIDINE:Polycystic ovaries:0.58), (COLCHICINE:Polycystic ovaries:0.17), (CORTISONEHYDROCORTISONE:Polycystic ovaries:0.02), (DACLIZUMAB:Polycystic ovaries:0.12), (DICLOFENAC:Polycystic ovaries:0.52), (DIGOXIN:Polycystic ovaries:0.04), (DRONABINOL:Polycystic ovaries:0.04), (ENOXAPARIN:Polycystic ovaries:0.07), (ESTRADIOLNORETHINDRONE ACETATE:Polycystic ovaries:0.25), (FOLLICLE STIMULATING HORMONE BETA POLYPEPTIDELUTROPIN ALFA:Polycystic ovaries:0.39), (HYDROCHLOROTHIAZIDELISINOPRIL:Polycystic ovaries:0.45), (HYDROXYZINE PAMOATE:Polycystic ovaries:0.88), (IBRUTINIB:Polycystic ovaries:0.08), (IMIPRAMINE HYDROCHLORIDE:Polycystic ovaries:0.2), (INDOMETHACIN:Polycystic ovaries:0.19), (ISOSORBIDE MONONITRATE:Polycystic ovaries:0.53), (LINAGLIPTIN:Polycystic ovaries:0.07), (METHYLPREDNISOLONE ACETATE:Polycystic ovaries:0.03), (METHYLPREDNISOLONE SODIUM SUCCINATE:Polycystic ovaries:0.48), (NICOTINE:Polycystic ovaries:0.1), (NITROFURANTOIN:Polycystic ovaries:0.22), (NITROFURANTOINNITROFURANTOIN MONOHYDRATE:Polycystic ovaries:0.14), (PANTOPRAZOLE MAGNESIUM:Polycystic ovaries:0.34), (PAROXETINE:Polycystic ovaries:0.5), (PENICILLIN:Polycystic ovaries:0.28), (PHENTERMINE HYDROCHLORIDE:Polycystic ovaries:0.04), (POLYETHYLENE GLYCOLS:Polycystic ovaries:0.21), (PRAMIPEXOLE DIHYDROCHLORIDE:Polycystic ovaries:0.49), (PRAVASTATIN SODIUM:Polycystic ovaries:0.27), (PRIMIDONE:Polycystic ovaries:0.52), (RANOLAZINE:Polycystic ovaries:0.23), (SOTALOL:Polycystic ovaries:0.1), (TAMSULOSIN:Polycystic ovaries:0.66), (TAMSULOSIN HYDROCHLORIDE:Polycystic ovaries:0.19), (TERIFLUNOMIDE:Polycystic ovaries:0.71), (TETRACYCLINE:Polycystic ovaries:0.38), (TOCILIZUMAB:Polycystic ovaries:0.47), (UMECLIDINIUM BROMIDEVILANTEROL TRIFENATATE:Polycystic ovaries:0.24), (IBUPROFEN:Polycythaemia:0.07), (NAPROXEN:Polycythaemia:0.15), (TORSE MIDE:Polycythaemia:0.01), (ACARBOSE:Polycythaemia vera:0.15), (ACETAMINOPHENOXYCODONEOXYCODONE HYDROCHLORIDE:Polycythaemia vera:0.53), (ACETAMINOPHENPROPOXYPHENE HYDROCHLORIDE:Polycythaemia vera:0.09), (AGALSIDASE BETA:Polycythaemia vera:0.34), (ALENDRONIC ACID:Polycythaemia vera:0.56), (ALISKIREN HEMIFUMARATE:Polycythaemia vera:0.35), (ALUMINUM HYDROXIDEDIMETHICONEMAGNESIUM HYDROXIDE:Polycythaemia vera:0.32), (AMINO ACIDSDEXTROSEELECTROLYTES NOS:Polycythaemia vera:0.12), (AMLODIPINE BESYLATEHYDROCHLOROTHIAZIDEVALSARTAN:Polycythaemia vera:0.04), (AMLODIPINE BESYLATEOLMESARTAN MEDOXOMIL:Polycythaemia vera:0.41), (AMLODIPINE BESYLATEVALSARTAN:Polycythaemia vera:0.1), (AMLODIPINEVALSARTAN:Polycythaemia vera:0.08), (AMPHOTERICIN B:Polycythaemia vera:0.31), (ANASTROZOLE:Polycythaemia vera:1.42), (ARMODAFINIL:Polycythaemia vera:1.34), (BACITRACIN:Polycythaemia vera:0.07), (BENAZEPRILBENAZEPRIL HYDROCHLORIDE:Polycythaemia vera:0.69), (BISACODYL OR DOCUSATE SODIUM:Polycythaemia vera:1.36), (BORTEZOMIB:Polycythaemia vera:0.26), (BUDESONIDEFORMOTEROL FUMARATE:Polycythaemia vera:0.43), (CALCITONIN:Polycythaemia vera:0.25), (CALCIUM\MAGNESIUM:Polycythaemia vera:0.15), (CANAKINUMAB:Polycythaemia vera:0.09), (CAPTOPRIL:Polycythaemia vera:0.51), (CARFILZOMIB:Polycythaemia vera:0.46), (CLINDAMYCIN PHOSPHATE:Polycythaemia vera:0.02), (CORTICOTROPIN:Polycythaemia vera:0.16), (DACLIZUMAB:Polycythaemia vera:0.46), (DALTEPARIN:Polycythaemia vera:0.38), (DEFLAZACORT:Polycythaemia vera:0.23), (DESLORATADINE:Polycythaemia vera:0.87), (DEVICE:Polycythaemia vera:0.03), (DEXTROAMPHETAMINE SULFATE:Polycythaemia vera:0.03), (DIPYRIDAMOLE:Polycythaemia vera:0.06), (DOMPERIDONE:Polycythaemia vera:0.2), (DORZOLAMIDE:Polycythaemia vera:0.21), (DRONEDARONE:Polycythaemia vera:0.36), (DUTASTERIDETAMSULOSIN HYDROCHLORIDE:Polycythaemia vera:0.12), (EPLERENONE:Polycythaemia vera:0.65), (ESOMEPRAZOLE MAGNESIUMNAPROXEN:Polycythaemia vera:0.37), (ESTROGENS:Polycythaemia vera:0.02), (EVEROLIMUS:Polycythaemia vera:0.92), (FISH OIL\OMEGA-3 FATTY ACIDS:Polycythaemia vera:0.25), (FLUTICASONE FUROATESALMETEROL XINAFOATE:Polycythaemia vera:0.7), (FLUVOXAMINE MALEATE:Polycythaemia vera:0.13), (FORMOTEROL:Polycythaemia vera:0.29), (FUSIDIC ACID:Polycythaemia vera:0.43), (GLICLAZIDE:Polycythaemia vera:0.74), (HYDROCHLOROTHIAZIDEIRBESARTAN:Polycythaemia vera:1.24), (HYDROCHLOROTHIAZIDETELMISARTAN:Polycythaemia vera:0.51), (IDELALISIB:Polycythaemia vera:0.01), (ILOPROST:Polycythaemia vera:0.63), (IMIPRAMINE HYDROCHLORIDE:Polycythaemia vera:0.73), (INDAPAMIDEPERINDOPRIL:Polycythaemia vera:0.99), (IODINE:Polycythaemia vera:0.06), (LACTASE:Polycythaemia vera:0.36), (LEDIPASVIRSOFOSBUVIR:Polycythaemia vera:0.84), (LERCANIDIPINE HYDROCHLORIDE:Polycythaemia vera:1.22), (LIOTHYRONINE SODIUM:Polycythaemia vera:0.39), (LITHIUM CARBONATE:Polycythaemia vera:0.26), (LOMITAPIDE MESYLATE:Polycythaemia vera:0.34), (METHENAMINE HIPPURATE:Polycythaemia vera:0.49), (METHYLPREDNISOLONE ACETATE:Polycythaemia vera:0.97), (METHYLPREDNISOLONE SODIUM SUCCINATE:Polycythaemia vera:1.01), (MOXIFLOXACIN:Polycythaemia vera:0.21), (MOXIFLOXACIN HYDROCHLORIDE:Polycythaemia vera:1.19), (MOXONIDINE:Polycythaemia vera:0.19), (NILOTINIB:Polycythaemia vera:1.24), (NINTEDANIB:Polycythaemia vera:0.26), (OMEPRAZOLE SODIUM:Polycythaemia vera:0.11), (OXAZEPAM:Polycythaemia vera:0.24), (OXCARBAZEPINE:Polycythaemia vera:0.84), (PANTOPRAZOLE MAGNESIUM:Polycythaemia vera:1.27), (PATIROMER:Polycythaemia vera:0.26), (PAZOPANIB HYDROCHLORIDE:Polycythaemia vera:0.51), (PEGFILGRASTIM:Polycythaemia vera:0.04), (PEGINTERFERON BETA-1A:Polycythaemia vera:0.29), (PENICILLIN:Polycythaemia vera:0.96), (PENTOSAN POLYSULFATE SODIUM:Polycythaemia vera:0.17), (PHENOBARBITAL:Polycythaemia vera:0.19), (PRAZOSIN:Polycythaemia vera:0.08), (PRIMIDONE:Polycythaemia vera:1.07), (REGORAFENIB:Polycythaemia vera:0.04), (ROFLUMILAST:Polycythaemia vera:0.42), (SACUBITRILVALSARTAN:Polycythaemia vera:0.36), (SECUKINUMAB:Polycythaemia vera:0.55), (SELEXIPAG:Polycythaemia vera:0.24), (SENNA LEAFSENNOSIDESSENNOSIDES A AND B:Polycythaemia vera:0.43), (SODIUM OXYBATE:Polycythaemia vera:1.24), (TERBUTALINE SULFATE:Polycythaemia vera:0.76), (TINZAPARIN SODIUM:Polycythaemia vera:0.02), (TOCILIZUMAB:Polycythaemia vera:0.14), (TOFACITINIB CITRATE:Polycythaemia vera:0.08), (VALGANCICLOVIR HYDROCHLORIDE:Polycythaemia vera:0.92), (VITAMIN A:Polycythaemia vera:0.44), (ANASTROZOLE:Polymyalgia rheumatica:0.91), (BISACODYL:Polymyalgia rheumatica:0.13), (BISACODYL OR DOCUSATE SODIUM:Polymyalgia rheumatica:0.48), (BUDESONIDEFORMOTEROL FUMARATE:Polymyalgia rheumatica:0.12), (BUSPIRONE HYDROCHLORIDE:Polymyalgia rheumatica:0.37), (CALCIUM ACETATE:Polymyalgia rheumatica:0.21), (CARVEDILOL PHOSPHATE:Polymyalgia rheumatica:0.24), (CHOLESTYRAMINE:Polymyalgia rheumatica:0.48), (CILOSTAZOL:Polymyalgia rheumatica:0.07), (CLINDAMYCINCLINDAMYCIN PHOSPHATE:Polymyalgia rheumatica:0.2), (CLOTRIMAZOLE:Polymyalgia rheumatica:0.24), (CLOZAPINE:Polymyalgia rheumatica:0.19), (DABIGATRAN:Polymyalgia rheumatica:0.06), (DAPAGLIFLOZIN PROPANEDIOL:Polymyalgia rheumatica:0.05), (DASATINIB:Polymyalgia rheumatica:1.05), (DEVICE:Polymyalgia rheumatica:0.59), (DIETARY SUPPLEMENT:Polymyalgia rheumatica:0.04), (DIPHENHYDRAMINE:Polymyalgia rheumatica:0.07), (ENOXAPARIN:Polymyalgia rheumatica:0.62), (ESZOPICLONE:Polymyalgia rheumatica:0.46), (EVEROLIMUS:Polymyalgia rheumatica:1.06), (EVOLOCUMAB:Polymyalgia rheumatica:1.44), (FINGOLIMOD HYDROCHLORIDE:Polymyalgia rheumatica:0.43), (FLUTICASONE:Polymyalgia rheumatica:0.18), (FLUTICASONEFLUTICASONE PROPIONATE:Polymyalgia rheumatica:0.07), (FORMOTEROL FUMARATE:Polymyalgia rheumatica:0.27), (GLYCOPYRRONIUMINDACATEROL:Polymyalgia rheumatica:0.37), (HYDROMORPHONE:Polymyalgia rheumatica:0.01), (HYDROXYCHLOROQUINE DIPHOSPHATE:Polymyalgia rheumatica:0.07), (HYDROXYZINE PAMOATE:Polymyalgia rheumatica:0.02), (INDAPAMIDEPERINDOPRIL:Polymyalgia rheumatica:0.77), (INSULIN DETEMIR:Polymyalgia rheumatica:0.78), (IPRATROPIUM BROMIDE:Polymyalgia rheumatica:0.03), (LABETALOLLABETALOL HYDROCHLORIDE:Polymyalgia rheumatica:0.22), (METAXALONE:Polymyalgia rheumatica:0.16), (METHYLPREDNISOLONE ACETATE:Polymyalgia rheumatica:0.11), (MOXIFLOXACIN HYDROCHLORIDE:Polymyalgia rheumatica:0.14), (NAPROXEN SODIUM:Polymyalgia rheumatica:0.34), (NIACIN:Polymyalgia rheumatica:0.18), (NILOTINIB:Polymyalgia rheumatica:0.84), (OLODATEROL HYDROCHLORIDETIOTROPIUM BROMIDE MONOHYDRATE:Polymyalgia rheumatica:0.23), (OXCARBAZEPINE:Polymyalgia rheumatica:0.5), (OXYBUTYNIN CHLORIDE:Polymyalgia rheumatica:0.19), (PAZOPANIB HYDROCHLORIDE:Polymyalgia rheumatica:0.31), (PEMETREXED DISODIUM:Polymyalgia rheumatica:0.17), (PIOGLITAZONE HYDROCHLORIDE:Polymyalgia rheumatica:0.16), (PRAMIPEXOLE:Polymyalgia rheumatica:0.66), (PYRIDOXINE HYDROCHLORIDE:Polymyalgia rheumatica:0.06), (ROFLUMILAST:Polymyalgia rheumatica:0.02), (SUCRALFATE:Polymyalgia rheumatica:0.07), (TERIFLUNOMIDE:Polymyalgia rheumatica:0.51), (TIZANIDINE:Polymyalgia rheumatica:0.12), (TOLTERODINE TARTRATE:Polymyalgia rheumatica:0.32), (VARENICLINE TARTRATE:Polymyalgia rheumatica:0.09), (AMITRIPTYLINE:Polymyositis:0.13), (BUDESONIDE:Polymyositis:0.55), (CANDESARTAN CILEXETIL:Polymyositis:0.2), (CERTOLIZUMAB PEGOL:Polymyositis:0.82), (CLINDAMYCINCLINDAMYCIN PHOSPHATE:Polymyositis:0.04), (DEXLANSOPRAZOLE:Polymyositis:0.22), (DICLOFENAC: Polymyositis:0.14), (DIGOXIN:Polymyositis:0.6), (DIVALPROEX SODIUM:Polymyositis:0.02), (EVOLOCUMAB:Polymyositis:0.33), (FERROUS SULFATE:Polymyositis:0.21), (IBRUTINIB:Polymyositis:0.8), (ILOPROST:Polymyositis:0.07), (INSULIN HUMAN:Polymyositis:0.09), (ISOSORBIDE MONONITRATE:Polymyositis:0.07), (LOVASTATIN:Polymyositis:0.01), (NAPROXEN SODIUM:Polymyositis:0.07), (NEBIVOLOL:Polymyositis:0.34), (PRAVASTATIN:Polymyositi s:0.09), (RANOLAZINE:Polymyositis:0.09), (SACUBITRILVALSARTAN:Polymyositis:0.13), (TERIFLUNOMIDE:Polymyositis:0.4), (ALFACALCIDOL:Polyneuropathy:0.05), (AMIODARONE HYDROCHLORIDE:Polyneuropathy:1.14), (ARMODAFINIL:Polyneuropathy:0.21), (BENAZE PRIL HYDROCHLORIDE:Polyneuropathy:0.2), (BISACODYL OR DOCUSATE SODIUM:Polyneuropathy:0.56), (BUDESONIDEFORMOTEROL FUMARATE:Polyneuropathy:0.18), (CALCIUM ACETATE:Polyneuropathy:0.22), (CALCIUM CITRATE:Polyneuropathy:0.35), (CARVEDILOL PHOSPHATE:Polyneuropathy:0.42), (CEFTRIAXONE SODIUM:Polyneuropathy:0.14), (CEPHALEXIN:Polyneuropathy:1.24), (CLINDAMYCINCLI NDAMYCIN PHOSPHATE:Polyneuropathy:0.73), (CODEINE:Polyneuropathy:0.03), (COLCHICINE:Polyneu ropathy:0.55), (DASATINIB:Polyneuropathy:0.77), (DEXLANSOPRAZOLE:Polyneuropathy:0.94), (DIGITOXIN:Polyneuropathy:0.06), (DOFETILIDE:Polyneuropathy:0.09), (DOXYCYCLINE: Polyneuropathy:0.31), (DRONABINOL:Polyneuropathy:0.02), (ELETRIPTAN HYDROBROMIDE:Polyneuropathy:0.27), (ENALAPRIL MALEATE:Polyneuropathy:0.56), (ESTROGENS, CONJUGATED:Polyneuropathy:0.28), (ESZOPICLONE:Polyneuropathy:0.72), (EVOLOCUMA B:Polyneuropathy:0.91), (FLECAINIDE:Polyneuropathy:0.51), (FLUOXETINEFLUOXETINE HYDROCHLORIDE:Polyneuropathy:0.2), (HYDROMORPHONE HYDROCHLORIDE:Polyneuropathy:0.57), (ILOPROST:Polyneuropathy:0.34), (IMATINIB MESYLATE:Polyneuropathy:0.8), (INDAPAMIDE:Polyneuropathy:0.44), (INDAPAMIDEPER INDOPRIL:Polyneuropathy:0.51), (INDOMETHACIN:Polyneuropathy:0.09), (LABETALOLL ABETALOL HYDROCHLORIDE:Polyneuropathy:0.31), (LERCANIDIPINE HYDROCHLORIDE:Polyneuropathy:0.3), (LEUCOVORIN CALCIUM:Polyneuropathy:0.17), (LISDEXAMFETAMINE DIMESYLATE:Polyneuropathy:0.11), (LITHIUM CARBONATE:Polyneuropathy:0.48), (METAXALONE:Polyneuropathy:0.3), (METHENAMIN E HIPPURATE:Polyneuropathy:0.28), (METHYLPREDNISOLONE ACETATE:Polyneuropathy:0.29), (METOLAZONE:Polyneuropathy:0.13), (METRONIDAZOL EMETRONIDAZOLE HYDROCHLORIDE:Polyneuropathy:0.27), (MIDODRINE:Polyneuropathy:0.06), (OXAZEPA M:Polyneuropathy:0.11), (PANTOPRAZOLE MAGNESIUM:Polyneuropathy:0.83), (PAROXETINEPAROXETINE HYDROCHLORIDE:Polyneuropathy:0.33), (PEGINTERFERON BETA-1A:Polyneuropathy:0.01), (PEMETREXED DISODIUM:Polyneuropathy:0.02), (PERINDOPRIL:Polyneuropathy:0.48), (PIOGLITAZONE HYDROCHLORIDE:Polyneuropathy:0.02), (PROPAFENONE HYDROCHLORIDE:Polyneuropathy:0.04), (QUETIAPINE:Polyneuropathy:0.4), (RABEPRAZ OLE:Polyneuropathy:0.28), (RALOXIFENE HYDROCHLORIDE:Polyneuropathy:0.07), (RIVASTIGMINE TARTRATE:Polyneuropathy:0.21), (ROFLUMILAST:Polyneuropathy:0.21), (SACUBITRILVA LSARTAN:Polyneuropathy:1.17), (SCOPOLAMINE:Polyneuropathy:0.19), (SECUKINUMAB: Polyneuropathy:0.31), (SULFAMETHOXAZOLE:Polyneuropathy:0.01), (TERIFLUNOMIDE:P olyneuropathy:0.14), (TIMOLOL:Polyneuropathy:0.35), (TIOTROPIUM:Polyneuropathy:0.09), (UMECLIDINIUM BROMIDEVILANTEROL TRIFENATATE:Polyneuropathy:0.26), (ZONISAMIDE:Polyneuropathy:0.07), (ABIRATERON E ACETATE:Polyuria:0.04), (ADO-TRASTUZUMAB EMTANSINE:Polyuria:0.9), (AGALSIDASE BETA:Polyuria:0.48), (ALCOHOL:Polyuria:0.6), (ALEMTUZUMAB:Polyuria:0.28), (ALISKIR EN HEMIFUMARATE:Polyuria:0.03), (ALUMINUM HYDROXIDEDIMETHICONEMAGNESIUM HYDROXIDE:Polyuria:0.22), (AMISULPRIDE:Polyuria:0.96), (AMLODIPINEPERINDOPRIL: Polyuria:0.13), (ASPIRINDIPYRIDAMOLE:Polyuria:0.59), (ATRACURIUM:Polyuria:0.53), (AXITINIB:Polyuria:0), (AZILSARTAN KAMEDOXOMIL:Polyuria:0.52), (BENAZEPRILBENAZEPRIL HYDROCHLORIDE:Polyuria:0.87), (BLESELUMAB:Polyuria:0.66), (BLINATUMOMAB:Poly uria:0.18), (BRINZOLAMIDE:Polyuria:0.38), (BUPRENORPHINE HYDROCHLORIDENALOXONE HYDROCHLORIDE:Polyuria:0.59), (CALCIUM CARBONATEERGOCALCIFEROLRETINOL:Polyuria:0.29), (CALCIUM CARBONATEVITAMIN D:Polyuria:0.16), (CANAGLIFLOZINMETFORMIN HYDROCHLORIDE:Polyuria:0.8), (CANAKINUMAB:Polyuria:0), (CARVEDILOL PHOSPHATE:Polyuria:1.25), (CEFEPIMECEFEPIME HYDROCHLORIDE:Polyuria:0.19), (CICLESONIDE:Polyuria:0.48), (CISATRACURIUM:Poly uria:0.78), (CLINDAMYCIN:Polyuria:1.01), (CLOMIPRAMINE HYDROCHLORIDE:Polyuria:0.21), (CLOTRIMAZOLE:Polyuria:1.43), (CLOZAPINE:Polyuria: 1.61), (CORTICOTROPIN:Polyuria:0.24), (DACLIZUMAB:Polyuria:0.6), (DALFAMPRIDINE: Polyuria:0.44), (DANAZOL:Polyuria:0.48), (DAPAGLIFLOZIN PROPANEDIOL:Polyuria:0.57), (DAPSONE:Polyuria:1.19), (DARATUMUMAB:Polyuria:0.14), (DASATINIB:Polyuria:1.87), (DEFLAZACORT:Polyuria:0.63), (DESFLURANE:Polyuria:0.51), (DOXEPIN HYDROCHLORIDE:Polyuria:0.09), (DRONABINOL:Polyuria:0.91), (DUTASTERIDETAMS ULOSIN HYDROCHLORIDE:Polyuria:0), (ECONAZOLE:Polyuria:0.33), (ELETRIPTAN HYDROBROMIDE:Polyuria:1.02), (EMPAGLIFLOZINLINAGLIPTIN:Polyuria:0.33), (ERIBU LIN:Polyuria:0.13), (ESTROGENS, CONJUGATEDMEDROXYPROGESTERONE ACETATE:Polyuria:0.17), (FENOTEROL HYDROBROMIDEIPRATROPIUM BROMIDE:Polyuria:0.43), (FERROUS FUMARATE:Polyuria:0.16), (FERROUS SULFATEGLYCINE:Polyuria:0.49), (FLECAINIDE:Polyuria:1.74), (FLUDROCORTISONE ACETATE:Polyuria:0.14), (FLUINDIONE:Polyuria:0.72), (FLUTICASONE:Polyuria:0.89), (FL UTICASONE FUROATESALMETEROL XINAFOATE:Polyuria:0.83), (FONDAPARINUX:Polyuria:0.08), (FONDAPARINUX SODIUM:Polyuria:0.25), (FULVESTRANT:Polyuria:0.57), (FUSIDIC ACID:Polyuria:0.09), (GIMERACILOTERACILTEGAFUR:Polyuria:0.31), (GLUCAGON:Pol yuria:0.15), (GLYCOPYRRONIUM:Polyuria:0.09), (GUSPERIMUS TRIHYDROCHLORIDE:Polyuria:0.3), (HYDROCHLOROTHIAZIDETELMISARTAN:Polyur ia:0.59), (HYDROMORPHONE HYDROCHLORIDE:Polyuria:1.21), (HYDROXYCHLOROQUINE DIPHOSPHATE:Polyuria:0.15), (HYDROXYUREA:Polyuria:0.43), (HYDROXYZINE PAMOATE:Polyuria:1.42), (IBRITUMOMAB TIUXETAN:Polyuria:0.15), (IDARUCIZUMAB:Polyuria:0.11), (IDELALISIB:Polyuria:0.12), (I MATINIB MESYLATE:Polyuria:1.05), (IMMUNE GLOBULIN NOS:Polyuria:0.06), (INDAPAMIDEPERINDOPRIL:Polyuria:1.5), (IOPAMIDOL:Polyuria:0.26), (IPRATROPIUM:Polyuria:1.55), (KETAMINE HYDROCHLORIDE:Polyuria:0.11), (LACOSAMIDE:Polyuria:0.06), (LERCANIDIPINE HYDROCHLORIDE:Polyuria:1.25), (LEVOLEUCOVORIN CALCIUM:Polyuria:0.96), (LINACLOTIDE:Polyuria:0.02), (LOMITAPIDE MESYLATE:Polyuria:0.04), (MEDROXYPROGESTERONE ACETATE:Polyuria:0.09), (MEPERIDINE:Polyuria:0.86), (METHENAMINE HIPPURATE:Polyuria:0.58), (METHENOLONE ENANTHATE:Polyuria:0.3), (METHOCARBAMOL:Polyuria:0.67), (MINOCYCLINEMINOC YCLINE HYDROCHLORIDE:Polyuria:0.95), (NALOXONE:Polyuria:0.06), (NALOXONE HYDROCHLORIDETILIDINE HYDROCHLORIDE:Polyuria:0.1), (NICOTINE:Polyuria:0.04), (NIRAPARIB:Polyuria:0.19), (N ITROFURANTOINNITROFURANTOIN MONOHYDRATE:Polyuria:0.04), (NOREPINEPHRINE BITARTRATE:Polyuria:0.73), (PAMIDRONATE DISODIUM:Polyuria:0.33), (PANITUMUMAB:Polyuria:0.22), (PANTOPRAZOLE MAGNESIUM:Polyuria:1.3), (PARICALCITOL:Polyuria:0.51), (PAROXETINE:Polyuria:0.97), (PATIROMER:Polyuria:0.18), (PEGINTERFERON BETA-1A:Polyuria:0.25), (PENICILLIN G:Polyuria:0.12), (PENICILLIN V:Polyuria:0), (PENTAMIDINE ISETHIONATE:Polyuria:0.24), (PILSICAINIDE HYDROCHLORIDE:Polyuria:0.09), (PIMOBENDAN:Polyuria:0.46), (PIROXICAM:Polyuria:0.31), (PREDNISOLONE SODIUM PHOSPHATE:Polyuria:0.08), (PROCHLORPERAZINE:Polyuria:0.15), (PROGESTERONE:Pol yuria:0.25), (PYRIDOSTIGMINE BROMIDE:Polyuria:0.05), (RALOXIFENE HYDROCHLORIDE:Polyuria:0.31), (REGORAFENIB:Polyuria:0.19), (RETINOL:Polyuria:0.17), (RIZATRIPTAN BENZOATE:Polyuria:0.5), (ROPIVACAINE:Polyuria:0.02), (SALMETEROL XINAFOATE:Polyuria:0.07), (SARPOGRELATE:Polyuria:0.02), (SELEXIPAG:Polyuria:0.62), (SEVELAMER HYDROCHLORIDE:Polyuria:0.81), (SODIUM CITRATE:Polyuria:0.33), (SODIUM FERROUS CITRATE:Polyuria:0.12), (SUMATRIPTAN:Polyuria:0.23), (TEMOZOLOMIDE:Polyuria:0.08), (TERBINAFINE HYDROCHLORIDE:Polyuria:0.35), (TERBUTALINE:Polyuria:0.61), (TETRACYCLINE:Polyu ria:0.4), (THIAMYLAL SODIUM:Polyuria:0.33), (TIZANIDINE:Polyuria:0.72), (TOBRAMYCIN:Polyuria:0.06), (TOCO PHEROL:Polyuria:0.05), (TOLTERODINE:Polyuria:0.07), (VARENICLINE TARTRATE:Polyuria:1.49), (VECURONIUM BROMIDE:Polyuria:1.04), (VITAMIN A:Polyuria:0.38), (ZONISAMIDE:Polyuria:0.07), (DIGOXIN:Poor peripheral circulation:0.11), (EVOLOCUMAB:Poor peripheral circulation:0.04), (INSULIN LISPRO:Poor peripheral circulation:0.1), (RAMIPRIL:Poor peripheral circulation:0.59), (BUSPIRONE HYDROCHLORIDE:Poor quality sleep:0.42), (CALCIUM CARBONATECHOLECALCIFEROL:Poor quality sleep:0.01), (CYCLOBENZAPRINE HYDROCHLORIDE:Poor quality sleep:0.12), (EVOLOCUMAB:Poor quality sleep:0.17), (HYDROMORPHONE HYDROCHLORIDE:Poor quality sleep:0.05), (LOVASTATIN:Poor quality sleep:0.06), (METHYLPREDNISOLONE SODIUM SUCCINATE:Poor quality sleep:0.2), (PHENOBARBITAL:Poor quality sleep:0.03), (PRAMIPEXOLE DIHYDROCHLORIDE:Poor quality sleep:0.26), (RAMIPRIL:Poor quality sleep:0.02), (WARFARIN:Poor quality sleep:0.24), (DILTIAZEM:Portal hypertension:0.55), (ARMODAFINIL:Post coital contraception:0.18), (OXCARBAZEPINE:Post coital contraception:0.11), (PAROXETINE:Post coital contraception:0.21), (SODIUM OXYBATE:Post coital contraception:0.11), (SUMATRIPTANSUMATRIPTAN SUCCINATE:Post coital contraception:0.29), (VARENICLINE TARTRATE:Post coital contraception:0.53), (ALFACALCIDOL:Post herpetic neuralgia:0.55), (AMIODARONE:Post herpetic neuralgia:1.24), (BISACODYL OR DOCUSATE SODIUM:Post herpetic neuralgia:0.72), (BRINZOLAMIDE:Post herpetic neuralgia:0.09), (BUMETANIDE:Post herpetic neuralgia:1.08), (CALCITRIOL:Post herpetic neuralgia:0.76), (CANDESARTAN CILEXETILHYDROCHLOROTHIAZIDE:Post herpetic neuralgia:0.47), (CARVEDILOL PHOSPHATE:Post herpetic neuralgia:0.43), (CHOLESTYRAMINE:Post herpetic neuralgia:0.68), (CILOSTAZOL:Post herpetic neuralgia:0.12), (CLOZAPINE:Post herpetic neuralgia:0.04), (COLCHICINE:Post herpetic neuralgia:0.59), (COLESEVELAM HYDROCHLORIDE:Post herpetic neuralgia:0.08), (DACLIZUMAB:Post herpetic neuralgia:0.29), (DASATINIB:Post herpetic neuralgia:0.63), (DESLORATADINE:Post herpetic neuralgia:0.23), (DESVENLAFAXINE SUCCINATE:Post herpetic neuralgia:0.05), (DEVICE:Post herpetic neuralgia:0.13), (DICLOFENAC:Post herpetic neuralgia:1.32), (DIVALPROEX SODIUM:Post herpetic neuralgia:0.66), (DOXAZOSIN MESYLATE:Post herpetic neuralgia:0.25), (DUTASTERIDE:Post herpetic neuralgia:0.32), (EDOXABAN TOSYLATE:Post herpetic neuralgia:0.07), (ENALAPRIL MALEATE:Post herpetic neuralgia:0.53), (ESZOPICLONE:Post herpetic neuralgia:0.96), (EVEROLIMUS:Post herpetic neuralgia:0.45), (EVOLOCUMAB:Post herpetic neuralgia:0.94), (FESOTERODINE FUMARATE:Post herpetic neuralgia:0.4), (FLECAINIDE:Post herpetic neuralgia:0.32), (HERBALSTURMERIC:Post herpetic neuralgia:0.21), (HYDRALAZINE HYDROCHLORIDE:Post herpetic neuralgia:0.59), (HYDROCHLOROTHIAZIDEVALSARTAN:Post herpetic neuralgia:0.31), (HYOSCYAMINE:Post herpetic neuralgia:0.28), (ILOPROST:Post herpetic neuralgia:0.31), (IMIPRAMINE HYDROCHLORIDE:Post herpetic neuralgia:0.42), (ISOSORBIDE:Post herpetic neuralgia:0.38), (LABETALOLLABETALOL HYDROCHLORIDE:Post herpetic neuralgia:0.43), (LERCANIDIPINE:Post herpetic neuralgia:0.74), (LIDOCAINEPRILOCAINE:Post herpetic neuralgia:0.07), (METHYLPHENIDATE HYDROCHLORIDE:Post herpetic neuralgia:0.2), (MODAFINIL:Post herpetic neuralgia:0.45), (NILOTINIB:Post herpetic neuralgia:0.5), (OMEGA-3-ACID ETHYL ESTERS:Post herpetic neuralgia:0.45), (PANTOPRAZOLE MAGNESIUM:Post herpetic neuralgia:0.46), (PAROXETINEPAROXETINE HYDROCHLORIDE:Post herpetic neuralgia:0.32), (PAZOPANIB HYDROCHLORIDE:Post herpetic neuralgia:0.01), (PENICILLIN:Post herpetic neuralgia:0.13), (PHENYTOIN:Post herpetic neuralgia:0.08), (PIROXICAM:Post herpetic neuralgia:0.02), (PRAMIPEXOLE:Post herpetic neuralgia:0.55), (QUETIAPINE:Post herpetic neuralgia:0.2), (RALOXIFENE HYDROCHLORIDE:Post herpetic neuralgia:0.4), (RANOLAZINE:Post herpetic neuralgia:0.88), (ROPINIROLE:Post herpetic neuralgia:0.02), (SACUBITRILVALSARTAN:Post herpetic neuralgia:0.87), (SECUKINUMAB:Post herpetic neuralgia:0.57), (SODIUM OXYBATE:Post herpetic neuralgia:1.02), (SOLIFENACIN SUCCINATE:Post herpetic neuralgia:0.14), (SULFAMETHOXAZOLE:Post herpetic neuralgia:0.08), (SUMATRIPTANSUMATRIPTAN SUCCINATE:Post herpetic neuralgia:0.21), (TERIFLUNOMIDE:Post herpetic neuralgia:0.71), (VALGANCICLOVIR HYDROCHLORIDE:Post herpetic neuralgia:0.15), (VILAZODONE HYDROCHLORIDE:Post herpetic neuralgia:0.04), (ZINC:Post herpetic neuralgia:0.09), (DILTIAZEM HYDROCHLORIDE:Post laminectomy syndrome:0.14), (DIVALPROEX SODIUM:Post laminectomy syndrome:0.19), (ESCITALOPRAM OXALATE:Post laminectomy syndrome:0.52), (EVOLOCUMAB:Post laminectomy syndrome:0.45), (IRON:Post laminectomy syndrome:0.07), (LANSOPRAZOLE:Post laminectomy syndrome:0), (PANTOPRAZOLE:Post laminectomy syndrome:0.05), (PHENOBARBITAL:Post laminectomy syndrome:0.09), (PRAMIPEXOLE DIHYDROCHLORIDE:Post laminectomy syndrome:0.02), (TERIFLUNOMIDE:Post laminectomy syndrome:0.19), (DEXLANSOPRAZOLE:Post procedural infection:0.03), (ENALAPRIL:Post procedural infection:0.09), (IBRUTINIB:Post procedural infection:0.14), (.ALPHA.1-PROTEINASE INHIBITOR HUMAN:Post-traumatic stress disorder:0.41), (ACARBOSE:Post-traumatic stress disorder:0.06), (ACETAMINOPHENOXYCODONEOXYCODONE HYDROCHLORIDE:Post-traumatic stress disorder:0.61), (ALBIGLUTIDE:Post-traumatic stress disorder:0.25), (AMANTADINE:Post-traumatic stress disorder:0.33), (ARIPIPRAZOLE LAUROXIL:Post-traumatic stress disorder:0.43), (AZELASTINE:Post-traumatic stress disorder:0.05), (AZELASTINE HYDROCHLORIDE:Post-traumatic stress disorder:0.33), (BETAMETHASONE DIPROPIONATECALCIPOTRIENECALCIPOTRIENE HYDRATE:Post-traumatic stress disorder:0.02), (BIFIDOBACTERIUM LONGUM SUBSP. INFANTIS:Post-traumatic stress disorder:0.26), (BIPERIDEN HYDROCHLORIDE:Post-traumatic stress disorder:0.06), (BUDESONIDEFORMOTEROL FUMARATE:Post-traumatic stress disorder:0.27), (BUTYLSCOPOLAMINE BROMIDE:Post-traumatic stress disorder:0.23), (CALCIUM CARBONATECHOLECALCIFEROL:Post-traumatic stress disorder:1.05), (CANAGLIFLOZINMETFORMIN HYDROCHLORIDE:Post-traumatic stress disorder:0.47), (CANDESARTAN CILEXETIL:Post-traumatic stress disorder:0.99), (CAPTOPRIL:Post-traumatic stress disorder:0.13), (CARVEDILOL PHOSPHATE:Post-traumatic stress disorder:0.77), (CETIRIZINE HYDROCHLORIDEPSEUDOEPHEDRINE HYDROCHLORIDE:Post-traumatic stress disorder:0.42), (CLINDAMYCINCLINDAMYCIN PHOSPHATE:Post-traumatic stress disorder:0.85), (CORTICOTROPIN:Post-traumatic stress disorder:0.33), (CORTISONEHYDROCORTISONE:Post-traumatic stress disorder:0.54), (DACLIZUMAB:Post-traumatic stress disorder:0.68), (DASATINIB:Post-traumatic stress disorder:0.48), (DESONIDE:Post-traumatic stress disorder:0.35), (DIMENHYDRINATE:Post-traumatic stress disorder:0.38), (DUTASTERIDE:Post-traumatic stress disorder:0.97), (EDOXABAN TOSYLATE:Post-traumatic stress disorder:0.08), (EMPAGLIFLOZINLINAGLIPTIN:Post-traumatic stress disorder:0.32), (ENALAPRIL MALEATE:Post-traumatic stress disorder:1.01), (ENOXAPARIN:Post-traumatic stress disorder:0.56), (EPLERENONE:Post-traumatic stress disorder:0.19), (ERYTHROPOIETIN:Post-traumatic stress disorder:0.09), (FORMOTEROL FUMARATE:Post-traumatic stress disorder:0.18), (FORMOTEROL FUMARATE\MOMETASONE FUROATE:Post-traumatic stress disorder:0.18), (GLYCOPYRRONIUMINDACATEROL:Post-traumatic stress disorder:0.17), (GUANFACINE:Post-traumatic stress disorder:1.24), (HYDROCHLOROTHIAZIDEIRBESARTAN:Post-traumatic stress disorder:0.28), (HYDROCODONE BITARTRATEIBUPROFEN:Post-traumatic stress disorder:0.05), (ILOPROST:Post-traumatic stress disorder:0.3), (IMIPRAMINE HYDROCHLORIDE:Post-traumatic stress disorder:0.62), (INSULIN GLULISINE:Post-traumatic stress disorder:0.12), (LACTASE:Post-traumatic stress disorder:0.13), (LACTULOSE:Post-traumatic stress disorder:0.05), (LERCANIDIPINE:Post-traumatic stress disorder:0.24), (LERCANIDIPINE HYDROCHLORIDE:Post-traumatic stress disorder:0.15), (LOMITAPIDE MESYLATE:Post-traumatic stress disorder:0.35), (METHENAMINE HIPPURATE:Post-traumatic stress disorder:0.52), (METHYLPREDNISOLONE ACETATE:Post-traumatic stress disorder:0.3), (METHYLPREDNISOLONE SODIUM SUCCINATE:Post-traumatic stress disorder:1.22), (NADOLOL:Post-traumatic stress disorder:0.7), (NALOXONE HYDROCHLORIDEOXYCODONE HYDROCHLORIDE:Post-traumatic stress disorder:0.25), (NILOTINIB:Post-traumatic stress disorder:0.34), (NINTEDANIB:Post-traumatic stress disorder:0.07), (PANTOPRAZOLE MAGNESIUM:Post-traumatic stress disorder:1.06), (PEGINTERFERON BETA-1A:Post-traumatic stress disorder:0.55), (PENTOSAN POLYSULFATE SODIUM:Post-traumatic stress disorder:0.32), (PHENTERMINE HYDROCHLORIDE:Post-traumatic stress disorder:0.09), (PIMECROLIMUS:Post-traumatic stress disorder:0.04), (PONATINIB:Post-traumatic stress disorder:0.01), (PROPAFENONE HYDROCHLORIDE:Post-traumatic stress disorder:0.08), (PROTRIPTYLINE HYDROCHLORIDE:Post-traumatic stress disorder:0.1), (PYRIDOXINE HYDROCHLORIDE:Post-traumatic stress disorder:0.2), (RABEPRAZOLE:Post-traumatic stress disorder:0.36), (RALOXIFENE HYDROCHLORIDE:Post-traumatic stress disorder:0.25), (RIVASTIGMINE TARTRATE:Post-traumatic stress disorder:0.28), (RIZATRIPTAN:Post-traumatic stress disorder:0.24), (SACUBITRILVALSARTAN:Post-traumatic stress disorder:0.6), (SAXAGLIPTIN HYDROCHLORIDE:Post-traumatic stress disorder:0.01), (SCOPOLAMINE:Post-traumatic stress disorder:1.04), (SECUKINUMAB:Post-traumatic stress disorder:0.8), (SULFAMETHOXAZOLE:Post-traumatic stress disorder:0.11), (TETRABENAZINE:Post-traumatic stress disorder:0.12), (TETRACYCLINE:Post-traumatic stress disorder:0.6), (TIMOLOL:Post-traumatic stress disorder:0.18), (TOCOPHEROL:Post-traumatic stress disorder:0.14), (UMECLIDINIUM BROMIDE:Post-traumatic stress disorder:0.02), (UMECLIDINIUM BROMIDEVILANTEROL TRIFENATATE:Post-traumatic stress disorder:1.41), (VALGANCICLOVIR HYDROCHLORIDE:Post-traumatic stress disorder:0.1), (NAPROXEN:Postmenopause:0.46), (BUPIVACAINEEPINEPHRINE:Postoperati ve analgesia:0.06), (DILTIAZEM HYDROCHLORIDE:Postoperative analgesia:0.02), (ENALAPRIL:Postoperative analgesia:0.12), (ESMOLOL HYDROCHLORIDE:Postoperative analgesia:0.64), (METHYLENE BLUE:Postoperative analgesia:0.08), (NAPROXEN:Postoperative analgesia:0.06), (THIAMYLAL SODIUM:Postoperative analgesia:0.16), (ALCAFTADINE:Postoperative care:0.2), (ALENDRONIC ACID:Postoperative care:0.23), (ATRACURIUM:Postoperative care:0.4), (AZITHROMYCIN MONOHYDRATE:Postoperative care:0.3), (BENOXINATE HYDROCHLORIDE:Postoperative care:0.14), (BISACODYL OR DOCUSATE SODIUM:Postoperative care:0.39), (CALCITONIN SALMON:Postoperative care:0.07), (CANAKINUMAB:Postoperative care:0.01), (CANDESARTAN CILEXETILHYDROCHLOROTHIAZIDE:Postoperative care:0.32), (CARBOXYMETHYLCELLULOSE SODIUMHYPROMELLOSES:Postoperative care:0.42), (CARFILZOMIB:Postoperative care:0.02), (CARVEDILOL PHOSPHATE:Postoperative care:0.34), (CEFTRIAXONE SODIUM:Postoperative care:0.49), (CERTOLIZUMAB PEGOL:Postoperative care:0.58), (CHOLESTYRAMINE:Postoperative care:0.34), (CLOTRIMAZOLE:Postoperative care:0.29), (DASATINIB:Postoperative care:0.32), (DESLORATADINE:Postoperative care:0.03), (DEXTRAN 70GLYCERINHYPROMELLOSES:Postoperative care:0.09), (DIMENHYDRINATE:Postoperative care:0.11), (DOXAZOSIN MESYLATE:Postoperative care:0.08), (DUTASTERIDE:Postoperative care:0.19), (ENALAPRIL MALEATE:Postoperative care:0.38), (ESMOLOL HYDROCHLORIDE:Postoperative care:0.13), (ESZOPICLONE:Postoperative care:0.49), (EVEROLIMUS:Postoperative care:0.42), (EVOLOCUMAB:Postoperative care:0.48), (FERROUS SULFATE:Postoperative care:0.73), (FLUTICASONE:Postoperative care:0.13), (HOMATROPINEHOMATROPINE HYDROBROMIDE:Postoperative care:0.19), (HYDROXYCHLOROQUINE DIPHOSPHATE:Postoperative care:0.28), (HYDROXYZINE PAMOATE:Postoperative care:0.33), (IBRUTINIB:Postoperative care:0.85), (IMIPRAMINE HYDROCHLORIDE:Postoperative care:0.1), (INDAPAMIDEPERINDOPRIL:Postoperative care:0.22), (LERCANIDIPINE:Postoperative care:0.42), (LIDOCAINEPRILOCAINE:Postoperative care:0.03), (LOVASTATIN:Postoperative care:1.09), (MELATONIN:Postoperative care:0.44), (METAXALONE:Postoperative care:0.55), (METHENAMINE HIPPURATE:Postoperative care:0.03), (METHYLPREDNISOLONE SODIUM SUCCINATE:Postoperative care:0.88), (METRONIDAZOLEMETRONIDAZOLE HYDROCHLORIDE:Postoperative care:0.28), (MOXIFLOXACINTRIAMCINOLONE:Postoperative care:1.04), (NIACIN:Postoperative care:0.01), (NILOTINIB:Postoperative care:0.42), (NITROFURANTOIN:Postoperative care:0.4), (NORTRIPTYLINE:Postoperative care:0.11), (OLODATEROL HYDROCHLORIDETIOTROPIUM BROMIDE MONOHYDRATE:Postoperative care:0.03), (OXCARBAZEPINE:Postoperative care:0.98), (OXYBUTYNIN CHLORIDE:Postoperative care:0.11), (PANTOPRAZOLE MAGNESIUM:Postoperative care:0.11), (PAROXETINE:Postoperative care:0.4), (PAROXETINEPAROXETINE HYDROCHLORIDE:Postoperative care:0.1), (PHENOBARBITAL:Postoperative care:0.22), (POLYETHYLENE GLYCOL 400POLYVINYL ALCOHOL:Postoperative care:0.07), (PONATINIB:Postoperative care:0.05), (PREDNISOLONEPREDNISOLONE ACETATEPREDNISOLONE SODIUM SUCCINATE:Postoperative care:0.13), (PRIMIDONE:Postoperative care:0.4), (PROPYLENE GLYCOL:Postoperative care:0.24), (PYRIDOXINE:Postoperative care:0.11), (RALOXIFENE HYDROCHLORIDE:Postoperative care:0.07), (REMIFENTANIL:Postoperative care:0.81), (RIBOFLAVIN 5′-PHOSPHATE SODIUM:Postoperative care:0.86), (RIZATRIPTAN BENZOATE:Postoperative care:0.05), (ROPIVACAINE:Postoperative care:0.45), (ROPIVACAINE HYDROCHLORIDE:Postoperative care:0.01), (SECUKINUMAB:Postoperative care:0.18), (SUCCINYLCHOLINE CHLORIDE:Postoperative care:0.14), (SULFAMETHOXAZOLE:Postoperative care:0.24), (THALIDOMIDE:Postoperative care:0.32), (THIOPENTAL:Postoperative care:0.18), (TIMOLOL MALEATETRAVOPROST:Postoperative care:0.49), (TOFACITINIB CITRATE:Postoperative care:0.25), (TRIAMTERENE:Postoperative care:0.01), (VECURONIUM BROMIDE:Postoperative care:0.71), (ZOPICLONE:Postoperative care:0), (OMEPRAZOLE MAGNESIUM:Postural orthostatic tachycardia syndrome:0), (VARENICLINE TARTRATE:Postural orthostatic tachycardia syndrome:0.21), (ACETAMINOPHENCODEINE PHOSPHATE:Pregnancy:0.1), (ATORVASTATIN CALCIUM:Pregnancy:0.27), (CELECOXIB:Pregnancy:0.09), (CIPROFLOXACIN HYDROCHLORIDE:Pregnancy:0.18), (CLOBAZAM:Pregnancy:0.07), (CLONIDINE:Pregnanc y:0.06), (CYCLOBENZAPRINE:Pregnancy:0.23), (DALFAMPRIDINE:Pregnancy:0.89), (DIPH ENHYDRAMINE:Pregnancy:0.47), (DULOXETINE:Pregnancy:0.02), (MELATONIN:Pregnanc y:0.51), (PAROXETINE:Pregnancy:0.17), (PAROXETINEPAROXETINE HYDROCHLORIDE:Pregnancy:0.06), (QUETIAPINE:Pregnancy:0.28), (SIMVASTATIN:Pregn ancy:0.02), (TIZANIDINE HYDROCHLORIDE:Pregnancy:0.24), (TRAMADOL:Pregnancy:0.55), (WARFARIN SODIUM:Pregnancy:0.04), (ACETAMINOPHENDICHLORALPHENAZONEISOMETHEPT ENE:Premedication:0.03), (AJMALINE:Premedication:0.21), (ALBIGLUTIDE:Premedication:0.07), (ALBUTEROLGUAIFENESIN:Premedication:0.19), (ALIROCUMAB:Premedication:0.03), (ALISKIREN HEMIFUMARATE:Premedication:1.05), (ALUMINUM HYDROXIDEMAGNESIUM OXIDEOXETHAZAINE:Premedication:1.17), (AMEZINIUM METILSULFATE:Premedication:0.04), (AMLODIPINE BESYLATEHYDROCHLOROTHIAZIDEOLMESARTAN MEDOXOMIL:Premedication:0.52), (AMLODIPINE BESYLATEHYDROCHLOROTHIAZIDEVALSARTAN:Premedication:1.19), (AMLODIPIN E BESYLATEIRBESARTAN:Premedication:0.28), (AMMONIUM CHLORIDEDEXTROMETHORPHAN\SODIUM CITRATE:Premedication:0.38), (AMOXAPINE:Premedication:0.21), (AMPICILLIN SODIUM:Premedication:0.39), (AMSACRINE:Premedication:0.34), (ANAGRELIDE:Premedica tion:0.41), (ANTIHEMOPHILIC FACTOR, HUMAN RECOMBINANT RESIDUES 743-1636 DELETED:Premedication:0.33), (ARSENIC TRIOXIDE:Premedication:0.99), (ASCORBIC ACID/CHROMIUM/SELENIUM/VANADYL SULFATE:Premedication:0.08), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Premedication:0.36), (ASCORBIC ACIDPOLYETHYLENE GLYCOL 3350POTASSIUM CHLORIDE\SODIUM ASCORBATE\SODIUM CHLORIDE\SODIUM SULFATE:Premedication:0.05), (ASIAN GINSENG:Premedication:0.14), (NIGER .ALPHA.-GALACTOSIDASE:Premedication:0.17), (ASPIRINBUTALBITALCAFFEINE:Premedication: 0.09), (ASPIRIN\CAFFEINE:Premedication:0), (ASPIRINDIHYDROXYALUMINUM AMINOACETATE ANHYDROUSMAGNESIUM CARBONATE:Premedication:1.04), (AZITHROMYCIN MONOHYDRATE:Premedication:0.17), (AZITHROMYCIN OR METHOTREXATE SODIUM:Premedication:1.76), (BAZEDOXIFENE ACETATEESTROGENS, CONJUGATED:Premedication:0.11), (BECLOMETHASONE DIPROPIONATEFORMOTEROL:Premedication:1.13), (BECLOMETHASONEFORMOTER OL:Premedication:0.5), (BELATACEPT:Premedication:1.09), (BEPRIDIL:Premedication:0.32), (BERAPROST:Premedication:0.93), (BETAHISTINE MESILATE:Premedication:0.45), (BETAMETHASONE DIPROPIONATECALCIPOTRIENE HYDRATE:Premedication:0.61), (BETAMETHASONECALCIPOTRIENE:Premedication:1.45), (LONGUM:Premedication:0.28), (BILASTINE:Premedication:1.73), (BIPERIDEN HYDROCHLORIDE:Premedication:0.09), (BLACK COHOSH:Premedication:0), (BLESELUMAB:Premedication:0.23), (BOCEPREVIR:Premedicati on:0.64), (BOCOCIZUMAB:Premedication:0.1), (BOSUTINIB:Premedication:0.7), (BOSUTINI B MONOHYDRATE:Premedication:0.02), (BOTULISM ANTITOXIN:Premedication:0.61), (BRINCIDOFOVIR:Premedication:0.33), (BROMFENAC SODIUM:Premedication:0.22), (CABOZANTINIB:Premedication:0.55), (CABOZANTINIB S-MALATE:Premedication:0.55), (CALCIUM ASPARTATE:Premedication:0.03), (CALCIUM\MAGNESIUM\ZINC:Premedication:0.74), (CA NAGLIFLOZINMETFORMIN HYDROCHLORIDE:Premedication:1.2), (CANAKINUMAB:Premedication:2.23), (CANNABI DIOL:Premedication:0.02), (CANRENOATE POTASSIUM:Premedication:0.32), (CARBIMAZOLE:Premedication:1.9), (CARBON DIOXIDE:Premedication:0.05), (CARTEOLOL:Premedication:0.41), (CARTEOLOL HYDROCHLORIDE:Premedication:0.59), (CARVEDILOL PHOSPHATE:Premedication:1.94), (CEFCAPENE PIVOXIL HYDROCHLORIDE HYDRATE:Premedication:0.51), (CERITINIB:Premedication:1.36), (CERTOLIZUMAB:Premed ication:0.31), (CHLORAL HYDRATE:Premedication:0.24), (CHLORHEXIDINE GLUCONATECHLOROBUTANOL:Premedication:0.04), (CHLOROQUINE PHOSPHATE:Premedication:0.58), (CHOLESTEROL:Premedication:0.11), (CHONDROITIN SULFATE (CHICKEN):Premedication:1.38), (CISAPRIDE:Premedication:0.42), (CLIKSTAR:Premedicatio n:0.21), (CLOFAZIMINE:Premedication:0.01), (COAGULATION FACTOR IX HUMANCOAGULATION FACTOR VII HUMANCOAGULATION FACTOR X HUMANPROTEIN CPROTEIN S HUMANPROTHROMBIN:Premedication:0.19), (COAGULATION FACTOR VIIA RECOMBINANT HUMAN:Premedication:0.74), (COLLAGEN:Premedication:0.02), (MAJALISCRATAEGUS LAEVIGATA LEAFPROXYPHYLLINE:Premedication:0.17), (CROTALIDAE POLYVALENT IMMUNE FAB (OVINE):Premedication:1.02), (CYANOCOBALAMINFOLIC ACIDPYRIDOXINE:Premedication:0.22), (CYCLOPHOSPHAMIDEDOXORUBICINPRED NISONERITUXIMABVINCRISTINE:Premedication:0.03), (CYPROTERONE:Premedication: 0.26), (DACLATASVIR:Premedication:0.06), (DACLIZUMAB:Premedication:2.98), (DAPAGLI FLOZIN:Premedication:0.22), (DAPAGLIFLOZIN PROPANEDIOLMETFORMIN HYDROCHLORIDE:Premedication:0.23), (DASABUVIROMBITASVIRPARITAPREVIRRI TONAVIR:Premedication:0.2), (DAUNORUBICIN CITRATE:Premedication:0.14), (DESVENLAFAXINE:Premedication:0.09), (DEXRAZOXANE: Premedication:1.12), (DEXTROAMPHETAMINE:Premedication:0.25), (DICLOXACILLIN:Pre medication:0.54), (DIENOGEST:Premedication:0.4), (DIFLUNISAL:Premedication:0.76), (DIHY DROCODEINE PHOSPHATE:Premedication:0.01), (DILAZEP:Premedication:0.13), (DIPHENIDOL HYDROCHLORIDE:Premedication:0.08), (DRONEDARONE:Premedication:0.94), (DROSPIR ENONEETHINYL ESTRADIOLLEVOMEFOLATE CALCIUM:Premedication:0.42), (DROXIDOPA:Premedication:0.12), (DUTASTERIDETAMS ULOSIN HYDROCHLORIDE:Premedication:0.82), (EDOXABAN TOSILATE MONOHYDRATE:Premedication:0.56), (ELTROMBOPAG OLAMINE:Premedication:0.63), (EMPAGLIFLOZINLINAGLIPTIN:Premedication:0.69), (EN ALAPRIL MALEATEHYDROCHLOROTHIAZIDE:Premedication:0.54), (EPACADOSTAT:Premedicati on:0.17), (ERIBULIN:Premedication:1.33), (ERYTHROMYCIN LACTOBIONATE:Premedication:0.18), (ETHIODIZED OIL:Premedication:0.37), (ETHYL ICOSAPENTATEICOSAPENT:Premedication:0.2), (ETRETINATE:Premedication:1.74), (FAR OPENEM SODIUM:Premedication:0.27), (FIBRINOGEN HUMAN:Premedication:0.12), (FILGRASTIM-SNDZ:Premedication:0.01), (FLAVIN ADENINE DINUCLEOTIDE:Premedication:0.31), (FLUNARIZINE HYDROCHLORIDE:Premedication:0.18), (FLUOCINOLONE ACETONIDE:Premedication:1.7), (FLUOROURACILIRINOTECANLEUCOVORIN:Premedi cation:0.03), (FLURAZEPAMFLURAZEPAM HYDROCHLORIDE:Premedication:0.23), (FONDAPARINUX:Premedication:1.57), (FOSPHEN YTOIN SODIUM:Premedication:0.89), (FUROSEMIDESPIRONOLACTONE:Premedication:0.04), (G ADOBENATE DIMEGLUMINE:Premedication:0.79), (GLIPIZIDEMETFORMIN HYDROCHLORIDE:Premedication:0.26), (GLYCOPYRROLATEINDACATEROL MALEATE:Premedication:1.01), (GOLD:Premedication:0.09), (GUANFACINE:Premedication:00.13), (HALOBETASOL PROPIONATE:Premedication:0.14), (HORSE CHESTNUT:Premedication:0.07), (HYDROCODONE BITARTRATEIBUPROFEN:Premedication:0.74), (HYDROXOCOBALAMINPYRIDOXINE HYDROCHLORIDETHIAMINE HYDROCHLORIDE:Premedication:0.09), (ICOSAPENT:Premedication:0.2), (IGURATIMOD:P remedication:0.33), (ILOPROST:Premedication:1.23), (IMIPRAMINE HYDROCHLORIDE:Premedication:0.9), (INSULIN PORKINSULIN PURIFIED PORK:Premedication:0.13), (ISONIAZIDPYRAZINAMIDERIFAMPIN:Premedication:0.99), (IXEKIZUMAB:Premedication:0.56), (KALLIDINOGENASE:Premedication:0.06), (LACTOBA CILLUS ACIDOPHILUSLACTOBACILLUS DELBRUECKII SUBSP. BULGARICUS:Premedication:0.91), (LANDIOLOL HYDROCHLORIDE:Premedication:0.25), (LANREOTIDE:Premedication:0.95), (LENVATINI B:Premedication:0.91), (LEVOMEFOLATE CALCIUM:Premedication:0.05), (LEVOTHYROXINE SODIUMPOTASSIUM IODIDE:Premedication:1.05), (LINSIDOMINE:Premedication:0.31), (LOMITAPIDE MESYLATE:Premedication:0.29), (LUTETIUM LU-177:Premedication:0.5), (LYSOZYME HYDROCHLORIDE:Premedication:0.28), (MAGNESIUM ASPARTATE HYDROCHLORIDE:Premedication:1.08), (MAGNESIUM PIDOLATE:Premedication:0.47), (MAGNESIUMPYRIDOXINE:Premedication:0.43), (MAPR OTILINE HYDROCHLORIDE:Premedication:0.23), (MELPERONE:Premedication:0.94), (MENTHOLZINC OXIDE:Premedication:0.49), (MEPOLIZUMAB:Premedication:1.59), (MESTRANOLNORETH INDRONE:Premedication:0.03), (METFORMIN HYDROCHLORIDESAXAGLIPTIN:Premedication:0.37), (METHENAMINE:Premedication:00.11), (METHYLPREDNISOLONE OR METHYLPREDNISOLONE ACETATE:Premedication:0.66), (METRELEPTIN:Premedication:0.42), (METYRAPONE:Prem edication:0.24), (MEXILETINE HYDROCHLORIDE:Premedication:0.33), (MIDOSTAURIN:Premedication:0.16), (MINERALS: Premedication:0.49), (MINODRONIC ACID:Premedication:0.49), (MOEXIPRIL HYDROCHLORIDE:Premedication:0.59), (MOGAMULIZUMAB:Premedication:0.93), (MORP HINE\MORPHINE HYDROCHLORIDE\MORPHINE SULFATE\MORPHINE TARTRATE:Premedication:0.1), (NARATRIPTAN HYDROCHLORIDE:Premedication:0.88), (NATEGLINIDE:Premedication:1.3), (NIMODIPINE: Premedication:0.62), (NIRAPARIB:Premedication:1.18), (NISOLDIPINE:Premedication:0.02), (NUSINERSEN:Premedication:0.71), (OLODATEROL HYDROCHLORIDE:Premedication:0.56), (OSPEMIFENE:Premedication:0.81), (PALIFERMIN: Premedication:0.36), (PAMIDRONIC ACID:Premedication:0.09), (PANOBINOSTAT:Premedication:0.58), (PARATHYROID HORMONE:Premedication:0.08), (PASIREOTIDE:Premedication:0.87), (PATIROMER:Premedi cation:0.3), (PAZUFLOXACIN MESILATE:Premedication:0.42), (PEGZEREPOETIN ALFA:Premedication:0.07), (PERPHENAZINE:Premedication:0.06), (PILSICAINIDE HYDROCHLORIDE:Premedication:0.45), (PITOFENONE:Premedication:0.27), (PIZOTYLINE: Premedication:0.48), (POLYMYXIN:Premedication:0.2), (POLYMYXIN B SULFATETRIMETHOPRIM SULFATE:Premedication:0), (PORFIMER SODIUM:Premedication:0.66), (POTASSIUM CITRATETRISODIUM CITRATE DIHYDRATE:Premedication:1.17), (PRAMOXINE HYDROCHLORIDE:Premedication:0.14), (PROTRIPTYLINE HYDROCHLORIDE:Premedication:0.21), (PYRIDOXAL PHOSPHATE:Premedication:0.32), (PYRIMETHAMINE:Premedication:0.89), (RADIUM RA-223 DICHLORIDE:Premedication:0.57), (RANIMUSTINE:Premedication:0.9), (REGORAFENIB:Pr emedication:2.04), (RICOLINOSTAT:Premedication:0.07), (RILPIVIRINE HYDROCHLORIDE:Premedication:0.19), (RISEDRONIC ACID:Premedication:0.22), (RIVASTIGMINE:Premedication:1.15), (RUCAPARIB CAMSYLATE:Premedication:0.14), (RUPATADINE FUMARATE:Premedication:0.42), (RUXOLITINIB:Premedication:0.21), (SCOPOLAMINE HYDROBROMIDE:Premedication:0.13), (SELEGILINE:Premedication:0.13), (SERRAPEPTAS E:Premedication:0.33), (SIBUTRAMINE HYDROCHLORIDE:Premedication:0.41), (SILICON DIOXIDE:Premedication:0.33), (SODIUM BICARBONATE\SODIUM CHLORIDE:Premedication:0.25), (SODIUM OXYBATE:Premedication:1.23), (SOFOSBUVIRVELPATASVIR:Premedication:0.11), (SORB ITOL:Premedication:0.51), (SULFADIAZINE:Premedication:0.24), (TABALUMAB:Premedicati on:0.2), (TAZOBACTAM SODIUM:Premedication:0.38), (TEDUGLUTIDE:Premedication:0.41), (TEDUGLUTIDEWAT ER:Premedication:1.09), (TELOTRISTAT ETHYL:Premedication:0.38), (TENIPOSIDE:Premedication:0.44), (TERIFLUNOMIDE:Premedi cation:1.06), (TESAMORELIN:Premedication:0.05), (TESTOSTERONE CYPIONATE:Premedication:0.5), (THIAMYLAL:Premedication:0.29), (THIAMYLAL SODIUM:Premedication:1.5), (THIOGUANINE:Premedication:0.59), (THROMBOPOIETIN:Pre medication:0.02), (TIBOLONE:Premedication:0.33), (TICLOPIDINE:Premedication:0.97), (TILI DINE:Premedication:0.55), (TIMOLOL MALEATETRAVOPROST:Premedication:1.47), (TIMOLOLTRAVOPROST:Premedication:00.23), (TIPIRACIL HYDROCHLORIDETRIFLURIDINE:Premedication:0.38), (TISAGENLECLEUCEL:Premedic ation:0.72), (TOFACITINIB:Premedication:0.89), (TREPROSTINIL SODIUM:Premedication:0.34), (TRIHEXYPHENIDYL:Premedication:0.32), (TRIHEXYPHENI DYL HYDROCHLORIDE:Premedication:0.11), (TRIMIPRAMINE MALEATE:Premedication:1.35), (TULOBUTEROL:Premedication:0.16), (UMECLIDINIUM BROMIDE:Premedication:1.18), (VALDECOXIB:Premedication:0.57), (VANDETANIB:Preme dication:0.51), (YEAST:Premedication:0.64), (ZICONOTIDE ACETATE:Premedication:0.99), (ZINC:Premedication:1.68), (ZIPRASIDONE HYDROCHLORIDEZIPRASIDONE MESYLATE:Premedication:0.22), (BUDESONIDE:Premenstrual dysphoric disorder:0.02), (CERTOLIZUMAB PEGOL:Premenstrual dysphoric disorder:0.09), (DEXLANSOPRAZOLE:Premenstrual dysphoric disorder:0.13), (ENALAPRIL:Premenstrual dysphoric disorder:0.1), (INDOMETHACIN:Premenstrual dysphoric disorder:0.47), (METAXALONE:Premenstrual dysphoric disorder:0.14), (TERIFLUNOMIDE:Premenstrual dysphoric disorder:0.13), (BUPRENORPHINE HYDROCHLORIDE:Premenstrual syndrome:0.3), (BUPRENORPHINE HYDROCHLORIDENALOXONE HYDROCHLORIDE:Premenstrual syndrome:0.47), (CERTOLIZUMAB PEGOL:Premenstrual syndrome:0.95), (CHOLESTYRAMINE:Premenstrual syndrome:0.2), (DALFAMPRIDINE:Premenstrual syndrome:0.45), (DEXLANSOPRAZOLE:Premenstrual syndrome:0.94), (DICLOFENAC:Premenstrual syndrome:0.19), (ENOXAPARIN:Premenstrual syndrome:0.13), (FERROUS SULFATE:Premenstrual syndrome:0.44), (GLIMEPIRIDE:Premenstrual syndrome:0.13), (HYDROXYZINE PAMOATE:Premenstrual syndrome:0.91), (METHYLPREDNISOLONE ACETATE:Premenstrual syndrome:0.23), (OSPEMIFENE:Premenstrual syndrome:0.06), (OXCARBAZEPINE:Premenstrual syndrome:1.09), (POLYETHYLENE GLYCOLS:Premenstrual syndrome:0), (PRAVASTATIN:Premenstrual syndrome:0.17), (PRAVASTATIN SODIUM:Premenstrual syndrome:0.25), (PRIMIDONE:Premenstrual syndrome:0.43), (UMECLIDINIUM BROMIDEVILANTEROL TRIFENATATE:Premenstrual syndrome:0.01), (VARENICLINE TARTRATE:Premenstrual syndrome:1.39), (VILAZODONE HYDROCHLORIDE:Premenstrual syndrome:0.27), (VITAMIN B:Premenstrual syndrome:0.01), (ALBUTEROL SULFATEIPRATROPIUM BROMIDE:Preoperative care:0.02), (AMIODARONE:Preoperative care:0.14), (ATRACURIUM:Preoperative care:0.51), (BUMETANIDE:Preoperative care:0.22), (CALCIUM CARBONATECHOLECALCIFEROL:Preoperative care:0.07), (CANDESARTAN CILEXETIL:Preoperative care:0.03), (DICLOFENAC:Preoperative care:0.19), (ENALAPRIL:Preoperative care:1.02), (ESMOLOL HYDROCHLORIDE:Preoperative care:0.13), (EVEROLIMUS:Preoperative care:0.05), (FERROUS SULFATE:Preoperative care:0.3), (FLUTICASONE PROPIONATE:Preoperative care:1.1), (IBRUTINIB:Preoperative care:0.09), (METHYLPREDNISOLONE SODIUM SUCCINATE:Preoperative care:0.28), (MOXIFLOXACINTRIAMCINOLONE:Preoperative care:0.3), (NAPROXEN:Preoperative care:0.47), (PRAVASTATIN:Preoperative care:0.04), (RIBOFLAVIN 5′-PHOSPHATE SODIUM:Preoperative care:0.05), (SOTALOL:Preoperative care:0.04), (SUGAMMADEX:Preoperative care:0.4), (TORSEMIDE:Preoperative care:0.02), (VERAPAMIL HYDROCHLORIDE:Preoperative care:0.04), (NAFARELIN ACETATE:Prevention of premature ovulation:0.18), (AMIODARONE:Primary immunodeficiency syndrome:0.02), (ARMODAFINIL:Primary immunodeficiency syndrome:0.44), (CALCIUM CARBONATECHOLECALCIFEROL:Primary immunodeficiency syndrome:0.11), (CARVEDILOL:Primary immunodeficiency syndrome:0.04), (CERTOLIZUMAB PEGOL:Primary immunodeficiency syndrome:0.71), (CORTISONEHYDROCORTISONE:Primary immunodeficiency syndrome:0.11), (DOCUSATE SODIUM:Primary immunodeficiency syndrome:0.56), (DULOXETINE:Primary immunodeficiency syndrome:0.2), (ENALAPRIL:Primary immunodeficiency syndrome:0.63), (ENOXAPARIN:Primary immunodeficiency syndrome:0.1), (ESZOPICLONE:Primary immunodeficiency syndrome:0.43), (EVOLOCUMAB:Primary immunodeficiency syndrome:0.46), (FERROUS SULFATE:Primary immunodeficiency syndrome:0.23), (FORMOTEROL FUMARATE\MOMETASONE FUROATE:Primary immunodeficiency syndrome:0.08), (HYDROXYZINE PAMOATE:Primary immunodeficiency syndrome:0.1), (INDOMETHACIN:Primary immunodeficiency syndrome:0.01), (PANTOPRAZOLE MAGNESIUM:Primary immunodeficiency syndrome:0.12), (PHENOBARBITAL:Primary immunodeficiency syndrome:0.03), (PRIMIDONE:Primary immunodeficiency syndrome:0.28), (SUMATRIPTANSUMATRIPTAN SUCCINATE:Primary immunodeficiency syndrome:0.26), (TERIFLUNOMIDE:Primary immunodeficiency syndrome:0.08), (UMECLIDINIUM BROMIDEVILANTEROL TRIFENATATE:Primary immunodeficiency syndrome:0.05), (VARENICLINE TARTRATE:Primary immunodeficiency syndrome:0.48), (WARFARIN:Primary immunodeficiency syndrome:0.28), (DEXLANSOPRAZOLE:Primary myelofibrosis:0.03), (DILTIAZEM HYDROCHLORIDE:Primary myelofibrosis:0.36), (ESOMEPRAZOLE MAGNESIUM:Primary myelofibrosis:0.26), (FLUTICASONE PROPIONATE:Primary myelofibrosis:0.26), (IMATINIB MESYLATE:Primary myelofibrosis:0.27), (NAPROXEN:Primary myelofibrosis:0.03), (SACUBITRILVALSARTAN:Primary myelofibrosis:0.23), (UNSPECIFIED INGREDIENT:Primary myelofibrosis:0.68), (.ALPHA.1-PROTEINASE INHIBITOR HUMAN:Probiotic therapy:0.28), (ALBIGLUTIDE:Probiotic therapy:0.01), (AMLODIPINE BESYLATEHYDROCHLOROTHIAZIDEOLMESARTAN MEDOXOMIL:Probiotic therapy:0.1), (ANASTROZOLE:Probiotic therapy:0.86), (BISOPROLOL FUMARATE:Probiotic therapy:0.44), (CALCIUM ACETATE:Probiotic therapy:0.37), (CANDESARTAN:Probiotic therapy:0.16), (CANDESARTAN CILEXETIL:Probiotic therapy:0.17), (CARVEDILOL PHOSPHATE:Probiotic therapy:0.19), (CETIRIZINE HYDROCHLORIDEPSEUDOEPHEDRINE HYDROCHLORIDE:Probiotic therapy:0.03), (CINACALCET HYDROCHLORIDE:Probiotic therapy:0.21), (COLCHICINE:Probiotic therapy:0.69), (CYCLOBENZAPRINE:Probiotic therapy:0.72), (DACLIZUMAB:Probiotic therapy:0.12), (DAPAGLIFLOZIN PROPANEDIOL:Probiotic therapy:0.04), (DASATINIB:Probiotic therapy:0.31), (DEVICE:Probiotic therapy:0.22), (DOXAZOSINDOXAZOSIN MESYLATE:Probiotic therapy:0.12), (DUTASTERIDE:Probiotic therapy:0.01), (ENALAPRIL MALEATE:Probiotic therapy:0.28), (ENOXAPARIN:Probiotic therapy:0.39), (EVOLOCUMAB:Probiotic therapy:1.1), (EZETIMIBESIMVASTATIN:Probiotic therapy:0.49), (FESOTERODINE FUMARATE:Probiotic therapy:0), (FINGOLIMOD HYDROCHLORIDE:Probiotic therapy:0.4), (FLUTICASONEFLUTICASONE PROPIONATE:Probiotic therapy:0.32), (FORMOTEROL FUMARATE\MOMETASONE FUROATE:Probiotic therapy:0.07), (HERBALSTURMERIC:Probiotic therapy:0.28), (HYDROCHLOROTHIAZIDELOSARTAN POTASSIUM:Probiotic therapy:0.06), (HYDROCHLOROTHIAZIDEVALSARTAN:Probiotic therapy:0.26), (HYDROMORPHONE:Probiotic therapy:0.4), (HYDROXYCHLOROQUINE DIPHOSPHATE:Probiotic therapy:0.32), (ILOPROST:Probiotic therapy:0.33), (INDOMETHACIN:Probiotic therapy:0.2), (ISOSORBIDE:Probiotic therapy:0.29), (LIDOCAINE HYDROCHLORIDE:Probiotic therapy:0.06), (LIDOCAINEPRILOCAINE:Probiotic therapy:0.15), (LOMITAPIDE MESYLATE:Probiotic therapy:0.08), (METHENAMINE HIPPURATE:Probiotic therapy:0.18), (METHYLPREDNISOLONE ACETATE:Probiotic therapy:0.33), (MODAFINIL:Probiotic therapy:0.65), (NAPROXEN SODIUM:Probiotic therapy:0.58), (OXCARBAZEPINE:Probiotic therapy:0.44), (PANTOPRAZOLE MAGNESIUM:Probiotic therapy:0.91), (PAROXETINE:Probiotic therapy:0.21), (PRIMIDONE:Probiotic therapy:0.56), (RABEPRAZOLE:Probiotic therapy:0.06), (RISEDRONATE SODIUM:Probiotic therapy:0.65), (RIZATRIPTAN BENZOATE:Probiotic therapy:0.42), (ROFLUMILAST:Probiotic therapy:0.1), (SACUBITRILVALSARTAN:Probiotic therapy:0.26), (SUMATRIPTANSUMATRIPTAN SUCCINATE:Probiotic therapy:0.58), (TAMSULOSIN:Probiotic therapy:1.37), (TELMISARTAN:Probiotic therapy:0.07), (TERIFLUNOMIDE:Probiotic therapy:1.04), (TETRACYCLINE:Probiotic therapy:0.49), (TOLTERODINE TARTRATE:Probiotic therapy:0.24), (TRIAMTERENE:Probiotic therapy:0.3), (VARENICLINE TARTRATE:Probiotic therapy:1.2), (ACARBOSE:Procedural pain:0.27), (ACETAMINOPHENPROPOXYPHENE NAPSYLATE:Procedural pain:0.16), (AGALSIDASE BETA:Procedural pain:0.08), (ALFACALCIDOL:Procedural pain:0.49), (ALUMINUM HYDROXIDEDIMETHICONEMAGNESIUM HYDROXIDE:Procedural pain:0.21), (AMANTADINE:Procedural pain:0.14), (AMINO ACIDSDEXTROSEELECTROLYTES NOS:Procedural pain:0.09), (AMISULPRIDE:Procedural pain:0.42), (AZELASTINE:Procedural pain:0.07), (BENAZEPRIL HYDROCHLORIDE:Procedural pain:1.11), (BENAZEPRILBENAZEPRIL HYDROCHLORIDE:Procedural pain:0.29), (BICALUTAMIDE:Procedural pain:0.38), (BORTEZOMIB:Procedural pain:0.05), (BRINZOLAMIDE:Procedural pain:0.57), (BUDESONIDEFORMOTEROL FUMARATE:Procedural pain:0.17), (CALCITONIN SALMON:Procedural pain:0.52), (CALCITRIOL:Procedural pain:0.96), (CALCIUM CARBONATEVITAMIN D:Procedural pain:0.73), (CANAGLIFLOZINMETFORMIN HYDROCHLORIDE:Procedural pain:0.16), (CANAKINUMAB:Procedural pain:0.08), (CARFILZOMIB:Procedural pain:0.53), (CARVEDILOL PHOSPHATE:Procedural pain:0.76), (CLONIDINE HYDROCHLORIDE:Procedural pain:0.03), (DACLIZUMAB:Procedural pain:0.23), (DALTEPARIN:Procedural pain:0.62), (DASATINIB:Procedural pain:0.94), (DEXMEDETOMIDINE HYDROCHLORIDE:Procedural pain:0.77), (DIPYRIDAMOLE:Procedural pain:0.11), (DOXAZOSIN MESYLATE:Procedural pain:0.55), (DRONABINOL:Procedural pain:0.46), (DUTASTERIDE:Procedural pain:1.04), (ECONAZOLE:Procedural pain:0.21), (EMPAGLIFLOZINLINAGLIPTIN:Procedural pain:0.18), (EPLERENONE:Procedural pain:0.19), (ESTROGENS:Procedural pain:0.02), (EVEROLIMUS:Procedural pain:0.78), (EVOLOCUMAB:Procedural pain:0.62), (FERROUS SULFATEGLYCINE:Procedural pain:0.13), (FLUDROCORTISONE ACETATE:Procedural pain:0.26), (FLUTICASONE FUROATESALMETEROL XINAFOATE:Procedural pain:0.57), (FONDAPARINUX SODIUM:Procedural pain:0.08), (FORMOTEROL FUMARATE:Procedural pain:0.38), (FOSPHENYTOINFOSPHENYTOIN SODIUM:Procedural pain:0.45), (FULVESTRANT:Procedural pain:0.2), (GIMERACILOTERACILTEGAFUR:Procedural pain:0.09), (HERBALSTURMERIC:Procedural pain:0.31), (HYALURONATE SODIUM:Procedural pain:0.18), (HYDROCHLOROTHIAZIDETELMISARTAN:Procedural pain:0.07), (HYDROCORTISONE ACETATE:Procedural pain:0.44), (HYDROXYCHLOROQUINE DIPHOSPHATE:Procedural pain:0.27), (HYDROXYZINE PAMOATE:Procedural pain:1.4), (ILOPROST:Procedural pain:0.08), (IMATINIB MESYLATE:Procedural pain:0.85), (INSULIN GLULISINE:Procedural pain:0.12), (IRINOTECAN:Procedural pain:0.45), (IRINOTECAN HYDROCHLORIDE:Procedural pain:0.61), (LACOSAMIDE:Procedural pain:0.02), (LEDIPASVIRSOFOSBUVIR:Procedural pain:0.53), (LERCANIDIPINE:Procedural pain:0.95), (LERCANIDIPINE HYDROCHLORIDE:Procedural pain:0.65), (LEVOLEUCOVORIN CALCIUM:Procedural pain:0.16), (LITHIUM:Procedural pain:0.27), (MECLIZINEMECLIZINE HYDROCHLORIDE:Procedural pain:0.08), (METHENAMINE HIPPURATE:Procedural pain:0.66), (METHYLENE BLUE:Procedural pain:0.19), (METHYLPREDNISOLONE ACETATE:Procedural pain:1.25), (MICONAZOLE NITRATE:Procedural pain:0.06), (MINOCYCLINEMINOCYCLINE HYDROCHLORIDE:Procedural pain:0.31), (MOXIFLOXACIN:Procedural pain:0.5), (MOXONIDINE:Procedural pain:0.02), (MUPIROCIN:Procedural pain:0.23), (NADOLOL:Procedural pain:0.15), (NEBIVOLOL:Procedural pain:0.97), (NOREPINEPHRINE:Procedural pain:0.25), (NOREPINEPHRINENOREPINEPHRINE BITARTRATE:Procedural pain:0.2), (NORETHINDRONE:Procedural pain:0.53), (OLODATEROL HYDROCHLORIDETIOTROPIUM BROMIDE MONOHYDRATE:Procedural pain:0.2), (OMEGA-3-ACID ETHYL ESTERS:Procedural pain:0.81), (OSELTAMIVIR:Procedural pain:0.12), (PAMIDRONATE DISODIUM:Procedural pain:0.06), (PANTOPRAZOLE MAGNESIUM:Procedural pain:1.08), (PAZOPANIB HYDROCHLORIDE:Procedural pain:0.02), (PEGINTERFERON BETA-1A:Procedural pain:0.24), (PONATINIB:Procedural pain:0.52), (PRAMIPEXOLE:Procedural pain:0.75), (PREDNISOLONEPREDNISOLONE ACETATEPREDNISOLONE SODIUM SUCCINATE:Procedural pain:0.56), (PYRIDOXINE:Procedural pain:0.28), (RALOXIFENE HYDROCHLORIDE:Procedural pain:0.13), (ROFLUMILAST:Procedural pain:0.66), (SACUBITRILVALSARTAN:Procedural pain:0.68), (SECUKINUMAB:Procedural pain:0.41), (SITAGLIPTIN:Procedural pain:0.22), (SUCCINYLCHOLINE CHLORIDE:Procedural pain:1.23), (SUMATRIPTAN:Procedural pain:0.46), (TERBINAFINE HYDROCHLORIDE:Procedural pain:0.3), (TERBUTALINE:Procedural pain:0.45), (TETRACYCLINE:Procedural pain:0.16), (THALIDOMIDE:Procedural pain:0.34), (THIAMYLAL SODIUM:Procedural pain:0.27), (TIOTROPIUM:Procedural pain:0.38), (TRIAMCINOLONE:Procedural pain:0.02), (UMECLIDINIUM BROMIDEVILANTEROL TRIFENATATE:Procedural pain:0.48), (VINCRISTINE SULFATE:Procedural pain:0.1), (VITAMIN A:Procedural pain:0.23), (CALCIUM CARBONATECHOLECALCIFEROL:Proctalgia:0.28), (CETUXIMAB:Proctalgia:0.42), (CITA LOPRAM HYDROBROMIDE:Proctalgia:0.17), (ENALAPRIL:Proctalgia:0.09), (NILOTINIB:Proctalgia:0.26), (OMEPRAZOLE MAGNESIUM:Proctalgia:0.17), (PANTOPRAZOLE:Proctalgia:0.05), (THALIDOMIDE:Proctal gia:0.35), (TOPIRAMATE:Proctalgia:0.14), (CELECOXIB:Product use in unapproved indication:0.06), (CERTOLIZUMAB PEGOL:Product use in unapproved indication:0.48), (DOCUSATE SODIUM:Product use in unapproved indication:0.22), (EVOLOCUMAB:Product use in unapproved indication:0.02), (LOVASTATIN:Product use in unapproved indication:0.18), (TERIFLUNOMIDE:Product use in unapproved indication:0.14), (VARENICLINE TARTRATE:Product use in unapproved indication:0.28), (ANASTROZOLE:Product use issue:0.45), (BENAZEPRIL HYDROCHLORIDE:Product use issue:0.3), (BUMETANIDE:Product use issue:0.65), (CANDESARTAN CILEXETIL:Product use issue:0.47), (CANDESARTAN CILEXETILHYDROCHLOROTHIAZIDE:Product use issue:0.07), (CIPROFLOXACIN HYDROCHLORIDE:Product use issue:0.09), (CLINDAMYCINCLINDAMYCIN PHOSPHATE:Product use issue:0.14), (DAPAGLIFLOZIN PROPANEDIOL:Product use issue:0.05), (DARBEPOETIN ALFA:Product use issue:0.37), (DEVICE:Product use issue:0.21), (DEXLANSOPRAZOLE:Product use issue:0.92), (DUTASTERIDE:Product use issue:0.13), (ELETRIPTAN HYDROBROMIDE:Product use issue:0.02), (ENALAPRIL MALEATE:Product use issue:0.1), (ERYTHROPOIETIN:Product use issue:0.09), (EVEROLIMUS:Product use issue:0.15), (EVOLOCUMAB:Product use issue:1.05), (EZETIMIBE:Product use issue:0.11), (HEPARIN SODIUM:Product use issue:0.44), (HYDROCHLOROTHIAZIDEVALSARTAN:Product use issue:0.09), (HYDROXYZINE PAMOATE:Product use issue:0.22), (ILOPROST:Product use issue:0.32), (IMATINIB MESYLATE:Product use issue:0.02), (IPRATROPIUM:Product use issue:0.06), (ISOSORBIDE:Product use issue:0.27), (ISOSORBIDE MONONITRATE:Product use issue:0.29), (LATANOPROST:Product use issue:0.04), (LEDIPASVIRSOFOSBUVIR:Product use issue:0.01), (LITHIUM CARBONATE:Product use issue:0.37), (LOVASTATIN:Product use issue:1), (METHYLPREDNISOLONE SODIUM SUCCINATE:Product use issue:0.49), (NILOTINIB:Product use issue:0.14), (NYSTATIN:Product use issue:0.07), (PANTOPRAZOLE:Product use issue:1.07), (PAROXETINE:Product use issue:0.16), (PHENYTOIN:Product use issue:0.44), (PRAMIPEXOLE DIHYDROCHLORIDE:Product use issue:0.13), (PRAVASTATIN SODIUM:Product use issue:0.74), (PROBIOTICS NOS:Product use issue:0.19), (RANOLAZINE:Product use issue:0.99), (SACUBITRILVALSARTAN:Product use issue:0.48), (SECUKINUMAB:Product use issue:0.08), (SUMATRIPTANSUMATRIPTAN SUCCINATE:Product use issue:0.07), (TERIFLUNOMIDE:Product use issue:0.31), (TIZANIDINE HYDROCHLORIDE:Product use issue:0.36), (TRAMADOL HYDROCHLORIDE:Product use issue:0.05), (VARENICLINE TARTRATE:Product use issue:0.57), (VITAMIN B:Product use issue:0.04), (ZONISAMIDE:Product use issue:0.38), (.ALPHA.-TOCOPHEROL ACETATE, D-ASCORBIC ACIDCALCIUM PANTOTHENATEFOLIC ACIDMANGANESENIACINAMIDEPYRIDOXINE\RIBOFLAVINTHIAMINEVITAMIN A\ZINC OXIDE:Product used for unknown indication:1.22), (10-HYDROXYCAMPTOTHECIN:Product used for unknown indication:1.01), (ACETAMINOPHENCHLORPHENIRAMINE MALEATEDEXTROMETHORPHAN HYDROBROMIDEDOXYLAMINE SUCCINATEPHENYLEPHRINE HYDROCHLORIDE:Product used for unknown indication:1.03), (ADUCANUMAB:Product used for unknown indication:0.91), (ALCOHOLCHLORHEXIDINE GLUCONATE:Product used for unknown indication:1.07), (ALGINIC ACIDALUMINUM HYDROXIDEMAGNESIUM TRISILICATE:Product used for unknown indication:1.09), (ALLERGENIC EXTRACT-CANDIDA:Product used for unknown indication:0.59), (ALPHA-TOCOPHEROL ACETATEASCORBIC ACIDBIOTINCHOLECALCIFEROLCYANOCOBALAMINDEXPANTHENOLFOLIC ACIDNIACINAMIDEPHYTONADIONEPYRIDOXINE HYDROCHLORIDE\RIBOFLAVIN 5′-PHOSPHATE SODIUMTHIAMINE HYDROCHLORIDEVITAMIN A PALMITATE:Product used for unknown indication:1), (ALPHA-TOCOPHEROLCHOLECALCIFEROL:Product used for unknown indication:1.19), (ANHYDROUS CITRIC ACIDPOTASSIUM BICARBONATE\SODIUM BICARBONATE:Product used for unknown indication:1.18), (ANHYDROUS DEXTROSECALCIUM CHLORIDEMAGNESIUM CHLORIDE\SODIUM BICARBONATE\SODIUM CHLORIDE\SODIUM LACTATE:Product used for unknown indication:1.05), (APROTININ:Product used for unknown indication:1.09), (ARABICA COFFEE BEANATROPA BELLADONNACALCIUM PHOSPHATECHAMOMILE:Product used for unknown indication:1.18), (ARTICAINE HYDROCHLORIDEEPINEPHRINE HYDROCHLORIDE:Product used for unknown indication:0.6), (ARTICAINEEPINEPHRINE:Product used for unknown indication:1.12), (ASPERGILLUS FUMIGATUS:Product used for unknown indication:0.29), (ASPIRINDEXTROMETHORPHAN HYDROBROMIDEDOXYLAMINE SUCCINATEPHENYLEPHRINE BITARTRATE:Product used for unknown indication:1.12), (ATROPA BELLADONNACALCIUM PHOSPHATECHAMOMILECOFFEA ARABICA FRUIT:Product used for unknown indication:1.16), (AUREOBASIDIUM PULLULANS VAR. PULLUTANS:Product used for unknown indication:0.05), (AUTOLOGOUS CULTURED CHONDROCYTESSUS SCROFA COLLAGEN:Product used for unknown indication:1.13), (AVOBENZONEECAMSULEOCTOCRYLENE:Product used for unknown indication:1.24), (AZASETRON:Product used for unknown indication:1), (BACILLUS CALMETTE-GUERIN SUBSTRAIN CONNAUGHT LIVE ANTIGEN:Product used for unknown indication:0.56), (BENRALIZUMAB:Product used for unknown indication:1.01), (BEZLOTOXUMAB:Product used for unknown indication:1.05), (CALCIUM CHLORIDEDEXTROSE MONOHYDRATEHYDROCHLORIC ACIDMAGNESIUM CHLORIDE\SODIUM BICARBONATE\SODIUM CHLORIDE\SODIUM HYDROXIDE\SODIUM LACTATE:Product used for unknown indication:0.99), (CAMPHORATED PHENOL:Product used for unknown indication:1.06), (CARBOPROST:Product used for unknown indication:0.91), (CENTELLA ASIATICA:Product used for unknown indication:1.07), (CEP-9722:Product used for unknown indication:1.04), (CHLORPHENIRAMINE MALEATEIBUPROFENPSEUDOEPHEDRINE HYDROCHLORIDE:Product used for unknown indication:1.11), (CILENGITIDE:Product used for unknown indication:1.08), (CIXUTUMUMAB:Product used for unknown indication:1.05), (CURCUMINDIETARY SUPPLEMENT:Product used for unknown indication:1.01), (DACETUZUMAB:Product used for unknown indication:1.06), (DALOTUZUMAB:Product used for unknown indication:1.01), (DENILEUKIN DIFTITOX:Product used for unknown indication:1.1), (EFAVIRENZLAMIVUDINEZIDOVUDINE:Product used for unknown indication:0.96), (EMTRICITABINETENOFOVIRZIDOVUDINE:Product used for unknown indication:0.98), (EQUINE BOTULINUM NEUROTOXIN A/B/C/D/E/F/G IMMUNE FAB2:Product used for unknown indication:1.15), (EQUISETUM ARVENSE TOP:Product used for unknown indication:1.04), (ERGONOVINEOXYTOCIN:Product used for unknown indication:1.02), (EVOFOSFAMIDE:Product used for unknown indication:1.05), (FALDAPREVIR:Product used for unknown indication:1.14), (FARLETUZUMAB:Product used for unknown indication:0.99), (FECAL MICROBIOTA:Product used for unknown indication:0.88), (FOSBRETABULIN TROMETHAMINE:Product used for unknown indication:1.04), (GLYCERINPETROLATUMPHENYLEPHRINE HYDROCHLORIDEPRAMOXINE HYDROCHLORIDE:Product used for unknown indication:1.06), (GLYCOLIC ACIDSALICYLIC ACID:Product used for unknown indication:0.72), (GLYCOPYRRONIUMFORMOTEROL FUMARATE:Product used for unknown indication:1.03), (HEXAMINOLEVULINATE HYDROCHLORIDE:Product used for unknown indication:1.11), (HOMEOPATHICSZINC ACETATE ANHYDROUSZINC GLUCONATE:Product used for unknown indication:0.97), (HUMAN COLOSTRUM:Product used for unknown indication:1.18), (HYALURONIDASE (HUMAN RECOMBINANT):Product used for unknown indication:0.98), (IFOSFAMIDEMESNA:Product used for unknown indication:1.09), (INFLUENZA A VIRUS A/CALIFORNIA/7/2009 (H1N1) RECOMBINANT HEMAGGLUTININ ANTIGEN\INFLUENZA A VIRUS A/VICTORIA/361/2011 (H3N2) RECOMBINANT HEMAGGLUTININ ANTIGEN\INFLUENZA B VIRUS B/WISCONSIN/1/2010 RECOMBINANT HEMAGGLUTININ ANTIGEN:Product used for unknown indication:1.04), (INIPARIB:Product used for unknown indication:0.99), (IODINETOSITUMOMAB:Product used for unknown indication:1.02), (LESTAURTINIB:Product used for unknown indication:0.99), (LEVODOPAUNSPECIFIED INGREDIENT:Product used for unknown indication:1.07), (LOBAPLATIN:Product used for unknown indication:0.93), (MICONAZOLE NITRATE OR TERBINAFINE HYDROCHLORIDE OR TOLNAFTATE:Product used for unknown indication:1.29), (MITOGUAZONE:Product used for unknown indication:1.12), (MOXIFLOXACINTRIAMCINOLONE:Product used for unknown indication:0.79), (OLODATEROLTIOTROPIUM BROMIDE:Product used for unknown indication:1.08), (OMARIGLIPTIN:Product used for unknown indication:1.08), (OMEGA-3-CARBOXYLIC ACIDS:Product used for unknown indication:1.03), (OPICINUMAB:Product used for unknown indication:1.1), (OXYMETAZOLINE HYDROCHLORIDETETRACAINE HYDROCHLORIDE:Product used for unknown indication:0.96), (PRETOMANID:Product used for unknown indication:0.99), (QUERCUS SALICINA STEM:Product used for unknown indication:1.11), (REFAMETINIB:Product used for unknown indication:1.08), (RILOTUMUMAB:Product used for unknown indication:1.03), (ROMOSOZUMAB:Product used for unknown indication:1), (SALSOLA KALI POLLEN:Product used for unknown indication:0.1), (SAVOLITINIB:Product used for unknown indication:1.01), (TANESPIMYCIN:Product used for unknown indication:1.04), (TECHNETIUM TC-99M TILMANOCEPT:Product used for unknown indication:1.06), (URIDINE TRIACETATE:Product used for unknown indication:1.05), (VELPATASVIR:Product used for unknown indication:0.99), (VERUPREVIR:Product used for unknown indication:1.04), (VITAMIN A ACETATE:Product used for unknown indication:1.06), (ZINC ACETATE ANHYDROUSZINC GLUCONATE:Product used for unknown indication:0.98), (ZINC ACETATE\ZINC GLUCONATE:Product used for unknown indication:1.02), (.ALPHA.1-PROTEINASE INHIBITOR HUMAN:Productive cough:0.98), (ACETAMINOPHENBUTALBITALCAFFEINE:Productive cough:0.54), (ACETAMINOPHENOXYCODONEOXYCODONE HYDROCHLORIDE:Productive cough:0.44), (ACETAZOLAMIDE:Productive cough:0.88), (ADO-TRASTUZUMAB EMTANSINE:Productive cough:0.91), (ALBUTEROLIPRATROPIUM:Productive cough:0.23), (AMISULPRIDE:Productive cough:0.54), (AMLODIPINEVALSARTAN:Productive cough:0.3), (AMPHETAMINE ASPARTATEAMPHETAMINE SULFATEDEXTROAMPHETAMINE SACCHARATEDEXTROAMPHETAMINE SULFATE:Productive cough:0.7), (AMPICILLIN:Productive cough:0.81), (ARMODAFINIL:Productive cough:0.52), (ATOVAQUONE:Productive cough:0.7), (BICALUTAMIDE:Productive cough:0.04), (BIFIDOBACTERIUM SPP.:Productive cough:0.23), (BIMATOPROST:Productive cough:0.1), (BLESELUMAB:Productive cough:0.03), (BROMAZEPAM:Productive cough:0.29), (BUPRENORPHINE HYDROCHLORIDENALOXONE HYDROCHLORIDE:Productive cough:0.24), (BUTYLSCOPOLAMINE BROMIDE:Productive cough:0.38), (CALCITONIN SALMON:Productive cough:0.36), (CALCIUM ACETATE:Productive cough:0.79), (CALCIUM CARBONATEVITAMIN D:Productive cough:0.06), (CALCIUMVITAMIN D:Productive cough:0.91), (CANAGLIFLOZINMETFORMIN HYDROCHLORIDE:Productive cough:0.49), (CANAKINUMAB:Productive cough:0.2), (CARBIMAZOLE:Productive cough:0), (CARVEDILOL PHOSPHATE:Productive cough:0.73), (CASPOFUNGIN:Productive cough:0.04), (CEFEPIMECEFEPIME HYDROCHLORIDE:Productive cough:0.05), (CHLORPHENIRAMINEHYDROCODONE:Productive cough:0.03), (CHOLESTYRAMINE:Productive cough:0.6), (CISATRACURIUM:Productive cough:0.14), (CLOZAPINE:Productive cough:1.07), (COLCHICINE:Productive cough:0.77), (COLESEVELAM HYDROCHLORIDE:Productive cough:0.09), (CYCLOBENZAPRINE:Productive cough:0.67), (DABIGATRAN:Productive cough:0.31), (DABRAFENIB:Productive cough:0.25), (DACLIZUMAB:Productive cough:0.33), (DALTEPARIN:Productive cough:0.45), (DANAZOL:Productive cough:0.08), (DAPAGLIFLOZIN PROPANEDIOL:Productive cough:0.08), (DASATINIB:Productive cough:1.52), (DEVICE:Productive cough:0.23), (DICYCLOMINE HYDROCHLORIDE:Productive cough:0.56), (DIGITOXIN:Productive cough:0.08), (DOCUSATE CALCIUM:Productive cough:0.42), (DOFETILIDE:Productive cough:0.48), (DUTASTERIDE:Productive cough:1.04), (EDOXABAN TOSYLATE:Productive cough:0.99), (ELETRIPTAN HYDROBROMIDE:Productive cough:0.75), (ELOTUZUMAB:Productive cough:0.63), (EMPAGLIFLOZINLINAGLIPTIN:Productive cough:0.08), (EPINASTINE HYDROCHLORIDE:Productive cough:0.41), (EPIRUBICIN:Productive cough:1.12), (EVOLOCUMAB:Productive cough:0.74), (EXEMESTANE:Productive cough:0.25), (FERROUS SULFATEGLYCINE:Productive cough:0.1), (FESOTERODINE FUMARATE:Productive cough:0.54), (FLECAINIDE:Productive cough:1.32), (FLUNITRAZEPAM:Productive cough:0.22), (FLUOCINONIDE:Productive cough:0.1), (FLUTICASONE:Productive cough:0.85), (FULVESTRANT:Productive cough:0.26), (GIMERACILOTERACILTEGAFUR:Productive cough:0.61), (HYDROCHLOROTHIAZIDETELMISARTAN:Productive cough:0.16), (HYDROXYZINE PAMOATE:Productive cough:1.12), (IBRUTINIB:Productive cough:1.84), (IDELALISIB:Productive cough:0.24), (ILOPROST:Productive cough:0.54), (IMIPRAMINE HYDROCHLORIDE:Productive cough:0.72), (INDAPAMIDEPERINDOPRIL:Productive cough:0.89), (IOPAMIDOL:Productive cough:0.28), (IRINOTECAN:Productive cough:0.12), (IRINOTECAN HYDROCHLORIDE:Productive cough:0.84), (ISONIAZID:Productive cough:0.44), (IVACAFTORLUMACAFTOR:Productive cough:0.1), (KETOPROFEN:Productive cough:0.31), (LABETALOLLABETALOL HYDROCHLORIDE:Productive cough:1), (LACTOBACILLUS RHAMNOSUS:Productive cough:0.18), (LANDIOLOL HYDROCHLORIDE:Productive cough:0.03), (LAPATINIB DITOSYLATE:Productive cough:0.14), (LERCANIDIPINE:Productive cough:1.36), (LERCANIDIPINE HYDROCHLORIDE:Productive cough:0.51), (LETROZOLE:Productive cough:0.63), (LEUCOVORIN CALCIUM:Productive cough:0.73), (LISDEXAMFETAMINE DIMESYLATE:Productive cough:0.05), (LITHIUM CARBONATE:Productive cough:0.76), (LUMACAFTOR:Productive cough:0.45), (MELATONIN:Productive cough:0.7), (METAXALONE:Productive cough:0.57), (METFORMIN HYDROCHLORIDEVILDAGLIPTIN:Productive cough:0.3), (METHENOLONE ENANTHATE:Productive cough:0.16), (METHIMAZOLE:Productive cough:0.04), (METHOCARBAMOL:Productive cough:0.37), (METHYLPREDNISOLONE ACETATE:Productive cough:0.51), (METOLAZONE:Productive cough:0.28), (MICAFUNGIN:Productive cough:0.13), (MIDODRINE:Productive cough:0.02), (MINOCYCLINEMINOCYCLINE HYDROCHLORIDE:Productive cough:0.83), (MYCOPHENOLATE MOFETILMYCOPHENOLATE MOFETIL HYDROCHLORIDE:Productive cough:0.9), (NABUMETONE:Productive cough:0.13), (NATEGLINIDE:Productive cough:0.31), (NEBIVOLOL HYDROCHLORIDE:Productive cough:0.62), (NIRAPARIB:Productive cough:0.06), (NORTRIPTYLINE:Productive cough:0.36), (OLAPARIB:Productive cough:0.12), (OSELTAMIVIR:Productive cough:0.41), (OXAZEPAM:Productive cough:0.45), (PAROXETINE:Productive cough:0.71), (PEGINTERFERON ALFA-2B:Productive cough:0.01), (PENICILLIN G:Productive cough:0.17), (PHENTOLAMINE:Productive cough:0.11), (PIMOBENDAN:Productive cough:0.26), (PRAVASTATIN:Productive cough:1.8), (PRIMIDONE:Productive cough:1.14), (RAMUCIRUMAB:Productive cough:0.53), (RANOLAZINE:Productive cough:0.73), (REGORAFENIB:Productive cough:0.47), (RETINOL:Productive cough:0.08), (SACUBITRILVALSARTAN:Productive cough:0.78), (SALMETEROL:Productive cough:0.1), (SECUKINUMAB:Productive cough:0.82), (SODIUM CITRATE:Productive cough:0.34), (SUCCINYLATED GELATIN:Productive cough:0.23), (SUNITINIB MALATE:Productive cough:0.09), (SUPLATAST TOSILATE:Productive cough:0.16), (THALIDOMIDE:Productive cough:1.49), (THEOPHYLLINE:Productive cough:0.25), (THIAMYLAL SODIUM:Productive cough:0.13), (TINZAPARIN:Productive cough:0.22), (TINZAPARIN SODIUM:Productive cough:0.65), (TIZANIDINE HYDROCHLORIDE:Productive cough:0.79), (TRAMETINIB:Productive cough:0.32), (UMECLIDINIUM BROMIDE:Productive cough:0.2), (VITAMIN A:Productive cough:0.24), (ZINC:Productive cough:0.18), (BICALUTAMIDE:Prophylactic chemotherapy:0.03), (BUPROPION HYDROCHLORIDE:Prophylactic chemotherapy:0.69), (CALCITRIOL:Prophylactic chemotherapy:0.2), (CANDESARTAN CILEXETILHYDROCHLOROTHIAZIDE:Prophylactic chemotherapy:0.48), (CEPHALEXIN:Prophylactic chemotherapy:0.75), (CRIZOTINIB:Prophylactic chemotherapy:0.01), (CYCLOBENZAPRINE:Prophylactic chemotherapy:0), (DEXLANSOPRAZOLE:Prophylactic chemotherapy:0.75), (DICLOFENAC:Prophylactic chemotherapy:0.21), (DRONABINOL:Prophylactic chemotherapy:0.03), (DUTASTERIDE:Prophylactic chemotherapy:0.02), (ERYTHROPOIETIN:Prophylactic chemotherapy:0.2), (ESZOPICLONE:Prophylactic chemotherapy:0.11), (EVEROLIMUS:Prophylactic chemotherapy:0.59), (EVOLOCUMAB:Prophylactic chemotherapy:0.12), (FERROUS SULFATE:Prophylactic chemotherapy:0.63), (FLECAINIDE:Prophylactic chemotherapy:0.1), (GLIPIZIDE:Prophylactic chemotherapy:0.25), (INDAPAMIDEPERINDOPRIL:Prophylactic chemotherapy:0.2), (LEDIPASVIRSOFOSBUVIR:Prophylactic chemotherapy:0.05), (LERCANIDIPINE HYDROCHLORIDE:Prophylactic chemotherapy:0.14), (NILOTINIB:Prophylactic chemotherapy:1.01), (PAMIDRONATE DISODIUM:Prophylactic chemotherapy:0.06), (PONATINIB:Prophylactic chemotherapy:0.11), (SACUBITRILVALSARTAN:Prophylactic chemotherapy:0.62), (TOCILIZUMAB:Prophylactic chemotherapy:0.07), (TRIAMCINOLONE ACETONIDE:Prophylactic chemotherapy:0.09), (VANCOMYCIN:Prophylactic chemotherapy:0.2), (.ALPHA.-TOCOPHEROL ACETATE, D-ASCORBIC ACIDCALCIUM PANTOTHENATEFOLIC ACIDMANGANESENIACINAMIDEPYRIDOXINE\RIBOFLAVINTHIAMINEVITAMIN A\ZINC OXIDE:Prophylaxis:0.33), (.ALPHA.-TOCOPHEROL.ALPHA.-TOCOPHEROL ACETATE.ALPHA.-TOCOPHEROL ACETATE, DL-ASCORBIC ACIDCHOLECALCIFEROLCOBALAMINCYANOCOBALAMINFLUORIDE IONFOLIC ACIDNIACINNIACINAMIDEPYRIDOXINEPYRIDOXINE HYDROCHLORIDE\RIBOFLAVIN\SODIUM ASCORBATE\SODIUM FLUORIDETHIAMINETHIAMINE MONONITRATEVITAMIN AVITA:Prophylaxis:0.05), (.ALPHA.-TOCOPHEROLASCORBIC ACID:Prophylaxis:0.16), (.BETA.-HYDROXYISOVALERIC ACIDARGININEGLUTAMINE:Prophylaxis:1.5), (10-HYDROXYCAMPTOTHECIN:Prophylaxis:0.43), (4-HYDROXYBUTANOIC ACID:Prophylaxis:0.13), (ABACAVIRLAMIVUDINEZIDOVUDINE:Prophylaxis:0.68), (ABA LOPARATIDE:Prophylaxis:0.37), (ABEMACICLIB:Prophylaxis:1.07), (ACEPROMAZINE:Pro phylaxis:0.57), (ACEPROMAZINE MALEATEACEPROMETAZINECLORAZEPATE DIPOTASSIUM:Prophylaxis:0.2), (ACETAMINOPHEN AND OR DEXTROMETHORPHAN AND OR DIPHENHYDRAMINE AND OR GUAIFENESIN AND OR PHENYLEPHRINE AND OR PSEUDOEPHEDRINE:Prophylaxis:0.49), (ACETAMINOPHEN OR ACETAMINOPHEN\ASPIRIN\CAFFEINE OR ASPIRIN\CAFFEINE:Prophylaxis:0.09), (ACETAMINOPHENAMOBARBITALCAFFEINE CODEINE PHOSPHATE:Prophylaxis:0.48), (ACETAMINOPHENASCORBIC ACIDPHENYLEPHRINE:Prophylaxis:0.18), (ACETAMINOPHEN\ASPIRIN:Prophylaxis:0.29), (ACETAMINOPHEN\ASPIRIN\CAFFEINECODEINE PHOSPHATE:Prophylaxis:0.27), (ACETAMINOPHEN\ASPIRINCODEINE PHOSPHATE:Prophylaxis:0.07), (ACETAMINOPHEN\ASPIRINSALICYLAMIDE:Prophylax is:0.66), (ACETAMINOPHENBELLADONNA LEAFCAFFEINE\MORPHINE:Prophylaxis:1.19), (ACETAMINOPHENBUCLIZINE HYDROCHLORIDECODEINE:Prophylaxis:0.02), (ACETAMINOPHENBUTYLSCOPOLAM INE BROMIDE:Prophylaxis:1.39), (ACETAMINOPHEN\CAFFEINEISOMETHEPTENE MUCATE:Prophylaxis:0.45), (ACETAMINOPHEN\CAFFEINEPROPOXYPHENE:Prophylaxi s:0.15), (ACETAMINOPHENCHLORPHENIRAMINE MALEATEDEXTROMETHORPHAN HYDROBROMIDE:Prophylaxis:0.12), (ACETAMINOPHENCHLORPHENIRAMINE MALEATEDEXTROMETHORPHAN HYDROBROMIDEPHENYLEPHRINE HYDROCHLORIDE OR ACETAMINOPHENDEXTROMETHORPHAN HYDROBROMIDEDOXYLAMINE SUCCINATEPHENYLEPHRINE HYDROCHLORIDE:Prophylaxis:0.56), (ACETAMINOPHENCHLORPHENIRAMINE MALEATEDEXTROMETHORPHAN HYDROBROMIDEPSEUDOEPHEDRINE HYDROCHLORIDE:Prophylaxis:0.53), (ACETAMINOPHENCHLORPHENIRAMINE MALEATEPHENYLEPHRINE HYDROCHLORIDE OR CETIRIZINE HYDROCHLORIDE OR CHLORPHENIRAMINE MALEATE OR CLEMASTINE FUMARATE OR DIPHENHYDRAMINE HYDROCHLORIDE OR FEXOFENADINE HYDROCHLORIDE OR LORATADINE:Prophylaxis:0.51), (ACETAMINOPHENCODEINE PHOSPHATEPROMETHAZINE HYDROCHLORIDE:Prophylaxis:0.26), (ACETAMINOPHENDEXTROMETHORPHAN HYDROBROMIDEGUAIFENESINPHENYLEPHRINE HYDROCHLORIDE:Prophylaxis:0.24), (ACETAMINOPHENDEXTROMETHORPHAN HYDROBROMIDEPHENYLEPHRINE HYDROCHLORIDE:Prophylaxis:0.42), (ACETAMINOPHENDEXTROMETHORPHAN HYDROBROMIDEPSEUDOEPHEDRINE HYDROCHLORIDE:Prophylaxis:0.41), (ACETAMINOPHENDEXTROMETHORPHANDOX YLAMINEPSEUDOEPHEDRINE:Prophylaxis:0.34), (ACETAMINOPHENDICHLORALPHE NAZONEISOMETHEPTENE HYDROCHLORIDE:Prophylaxis:0.51), (ACETAMINOPHENDIPHENHYDRAMINE HYDROCHLORIDEPHENYLEPHRINE HYDROCHLORIDE:Prophylaxis:0.09), (ACETAMINOPHENDIPHENHYDRAMINEDIPHE NHYDRAMINE CITRATE:Prophylaxis:0.23), (ACETAMINOPHENGUAIFENESINPHENYLEPHRINE HYDROCHLORIDE:Prophylaxis:0.36), (ACETAMINOPHENPHENYLEPHRINE HYDROCHLORIDE:Prophylaxis:0.71), (ACETAMINOPHENPSEUDOEPHEDRINE:Prophyla xis:0.41), (ACETIC ACIDCALCIUM CHLORIDEDEXTROSEMAGNESIUM CHLORIDE\POTASSIUM CHLORIDE\SODIUM CHLORIDE:Prophylaxis:0.59), (ACETIC ACIDDESONIDE:Prophylaxis:0.55), (ACETIC ACIDSODIUM ACETATE:Prophylaxis:1.03), (ACETOHYDROXAMIC ACID:Prophylaxis:0.59), (ACETONE:Prophylaxis:0.2), (ACETYLCHOLINE CHLORIDE:Prophylaxis:0.54), (ACETYLCYSTEINEBENZALKONIUM CHLORIDETUAMINOHEPTANE SULFATE:Prophylaxis:0.71), (ACETYLDIGITOXIN:Prophylaxis:0.75), (ACIPIMOX:Prophylax is:0.53), (ACRIVASTINE:Prophylaxis:0.44), (ACTIVATED CHARCOALDIMETHICONE:Prophylaxis:0.73), (ACYCLOVIRHYDROCORTISONE:Proph ylaxis:0.41), (AGALSIDASE ALFA:Prophylaxis:0.46), (AJMALINE:Prophylaxis:1.21), (ALANINEARGININEASPARTIC ACIDCYSTEINE HYDROCHLORIDEGLUTAMIC ACIDGLYCINEHISTIDINEISOLEUCINELEUCINELYSINE ACETATEMETHIONINEN-ACETYL-TYROSINEPHENYLALANINEPROLINESERINETAURINETHREONINETRYPTOPHA NTYROSINEVALINE:Prophylaxis:0.19), (ALANINEARGININEGLYCINEHISTIDINEIS OLEUCINELEUCINELYSINE HYDROCHLORIDEMETHIONINEPHENYLALANINEPROLINESERINETHREONINE TRYPTOPHANTYROSINEVALINE:Prophylaxis:0.38), (ALBUTEROLGUAIFENESIN:Prop hylaxis:1.21), (ALCOHOLALUMINUM CHLORIDE:Prophylaxis:0.37), (ALDOSTERONE:Prophylaxis:0.52), (ALEFACEPT:Prophylaxi s:0.3), (ALFALFA:Prophylaxis:0.64), (ALFALFAALFALFA WHOLE:Prophylaxis:0.44), (ALGINIC ACIDALUMINUM HYDROXIDEMAGNESIUM CARBONATE:Prophylaxis:0.11), (ALISERTIB:Prophylaxis:0.69), (ALISPORIVIR:Prophylaxis: 0.97), (ALLANTOIN:Prophylaxis:0.19), (ALLANTOINBENZOCAINECAMPHOR (NATURAL)DIMETHICONEMENTHOL:Prophylaxis:0.76), (ALLANTOINOCTENIDINE:P rophylaxis:0.11), (ALMOTRIPTAN:Prophylaxis:0.39), (ALOE:Prophylaxis:0.33), (ALOSETRON: Prophylaxis:0.65), (ALOSETRON HYDROCHLORIDE:Prophylaxis:0.53), (ALPHA-TOCOPHEROLASCORBIC ACIDCHOLECALCIFEROLCOPPERCYANOCOBALAMINFOLIC ACIDLUTEIN\OMEGA-3 FATTY ACIDSPYRIDOXINE HYDROCHLORIDETAURINEVITAMIN A PALMITATEZEAXANTHINZINC PICOLINATE:Prophylaxis:0.46), (ALPHA-TOCOPHEROLCHOLECALCIFEROL:Prophylaxis:0.68), (ALUMINUM CHLORIDE:Prophylaxis:0.32), (ALUMINUM HYDROXIDEALUMINUM PHOSPHATEGUAR GUM\MAGNESIUM HYDROXIDE:Prophylaxis:0.54), (ALVOCIDIB:Prophylaxis:0.77), (AMCINONIDE:Prophylaxis: 0.89), (AMILORIDE HYDROCHLORIDEHYDROCHLOROTHIAZIDETIMOLOL MALEATE:Prophylaxis:0.52), (AMINO ACIDSCALCIUM CHLORIDEMAGNESIUM CHLORIDE\SODIUM CHLORIDE\SODIUM LACTATE:Prophylaxis:0.61), (AMINOLEVULINIC ACID:Prophylaxis:0.48), (AMINOPYRINE:Prophylaxis:0.56), (AMITRIPTYLINECHLORDIA ZEPOXIDE:Prophylaxis:0.49), (AMLODIPINE MALEATEOLMESARTAN MEDOXOMIL:Prophylaxis:0.55), (AMLODIPINE MESYLATE:Prophylaxis:0.53), (AMLODIPINEHYDROCHLOROTHIAZIDEOLMESARTA N:Prophylaxis:0.99), (AMLODIPINELOSARTAN:Prophylaxis:1.02), (AMLODIPINELOSARTAN POTASSIUM:Prophylaxis:0.64), (AMOBARBITAL:Prophylaxis:0.17), (AMOROLFINE:Prophy laxis:0.65), (AMOXICILLINCLARITHROMYCINRABEPRAZOLE:Prophylaxis:0.2), (AMPH ETAMINE SULFATE:Prophylaxis:0.19), (AMPHETAMINEDEXTROAMPHETAMINE:Prophylaxis:0.35), (ANGIOTENSIN II:Prophylaxis:1.1), (ANHYDROUS CITRIC ACIDBICARBONATE IONMAGNESIUM CATION:Prophylaxis:0.08), (ANHYDROUS DEXTROSECALCIUM CHLORIDELACTIC ACIDMAGNESIUM CHLORIDE\POTASSIUM CHLORIDE\SODIUM BICARBONATE\SODIUM CHLORIDE:Prophylaxis:0.28), (ANHYDROUS DEXTROSECALCIUM CHLORIDEMAGNESIUM CHLORIDE\SODIUM BICARBONATE\SODIUM CHLORIDE\SODIUM LACTATE:Prophylaxis:0.7), (ANHYDROUS DEXTROSELACTIC ACIDMAGNESIUM CHLORIDE\POTASSIUM CHLORIDE\SODIUM BICARBONATE\SODIUM CHLORIDE:Prophylaxis:0), (ANTIHEMOPHILIC FACTOR PORCINE, B-DOMAIN TRUNCATED RECOMBINANT:Prophylaxis:0.46), (ANTIPYRINEBENZOCAINE:Prophylaxis:0.42), (APATI NIB:Prophylaxis:0.56), (APOMORPHINE HYDROCHLORIDE:Prophylaxis:0.61), (ARABICA COFFEE BEANATROPA BELLADONNACALCIUM PHOSPHATECHAMOMILE:Prophylaxis:0.11), (ARCTOSTAPHYLOS UVA-URSI LEAFURTICA DIOICA:Prophylaxis:0.77), (MONTANA:Prophylaxis:0.5), (AROTINOLOL:Prophylaxis:1.31), (ARSENIC:Prophylaxis:0.17), (ARTICAINE HYDROCHLORIDEEPINEPHRINE BITARTRATE:Prophylaxis:0.15), (ARTICAINEEPINEPHRINE:Prophylaxis:0.09), (ASCORBI C ACID/CHROMIUM/SELENIUM/VANADYL SULFATE:Prophylaxis:1.13), (ASCORBIC ACIDBIOTINCALCIUM PANTOTHENATEFERROUS ASPARTO GLYCINATEFERROUS FUMARATEFOLIC ACIDNIACINPYRIDOXINE HYDROCHLORIDE\RIBOFLAVINTHIAMINE MONONITRATE:Prophylaxis:0.6), (ASCORBIC ACIDCHONDROITIN SULFATE (BOVINE)GLUCOSAMINE SULFATEMANGANESE:Prophylaxis:0.64), (ASCORBIC ACIDCHROMIUMSELENIUMVANADYL SULFATE:Prophylaxis:0.49), (ASCORBIC ACIDCYANOCOBALAMINFOLIC ACIDIRON:Prophylaxis:0.91), (ASCORBIC ACIDFERROUS ASPARTO GLYCINATEFERROUS FUMARATEFOLIC ACIDNIACIN:Prophylaxis:0.63), (ASCORBIC ACIDFERROUS FUMARATEFOLIC ACID:Prophylaxis:0.94), (ASCORBIC ACIDNIACINAMIDEPYRIDOXINE HYDROCHLORIDE\RIBOFLAVINTHIAMINE HYDROCHLORIDE:Prophylaxis:0.22), (ASCORBIC ACIDNIACINAMIDEPYRIDOXINE\RIBOFLAVINTHIAMINE:Prophylaxis:0.09), (ASPAR TAME:Prophylaxis:0.18), (ASPIRIN SODIUM:Prophylaxis:0.63), (ASPIRIN\CAFFEINEORPHENADRINE CITRATE:Prophylaxis:0.68), (ASPIRIN\CAFFEINEPHENACETIN:Prophylaxis:0.93), (ASPIRI N\CAFFEINESALICYLAMIDE:Prophylaxis:0.55), (ASPIRINCHLORPHENIRAMINE MALEATEPHENYLEPHRINE BITARTRATE:Prophylaxis:0.47), (ASPIRINDEXTROMETHORPHAN HYDROBROMIDEDOXYLAMINE SUCCINATEPHENYLEPHRINE BITARTRATE:Prophylaxis:0.27), (ASPIRIN®XYCODONE HYDROCHLORIDE:Prophylaxis:0.28), (ASPIRINPRAVASTATIN:Prophylaxis:0.98), (ASPIRI N\SODIUM BICARBONATE:Prophylaxis:0.2), (ATENOLOL HYDROCHLORIDE:Prophylaxis:0.17), (ATENOLOLHYDROCHLOROTHIAZIDE:Prophylax is:0), (ATLANTIC SALMON OIL:Prophylaxis:0.24), (ATROPA BELLADONNACALCIUM PHOSPHATECHAMOMILECOFFEA ARABICA FRUIT:Prophylaxis:0.1), (ATROPINE SULFATEDIPHENOXYLATE:Prophylaxis:1.04), (ATROPINEDIPHENOXYLATE HYDROCHLORIDE:Prophylaxis:0.42), (AURANOFIN:Prophylaxis:0.58), (AUTOLOGOUS CULTURED CHONDROCYTESSUS SCROFA COLLAGEN:Prophylaxis:0.73), (AVANAFIL:Prophylaxis:1), (AVIBACTAM SODIUMCEFTAZIDIME:Prophylaxis:0.95), (AVOBENZONEHOMOSALATEOCTINOXA TEOCTISALATEOXYBENZONE:Prophylaxis:0.41), (AZITHROMYCIN MONOHYDRATE:Prophylaxis:1.87), (AZTREONAM LYSINE:Prophylaxis:0.12), (BACILLUS CALMETTE-GUERIN SUBSTRAIN CONNAUGHT LIVE ANTIGEN:Prophylaxis:0.91), (BACILLUS SUBTILIS:Prophylaxis:0.78), (BACITRACIN ZINCNEOMYCIN SULFATEPOLYMYXIN B SULFATEPRAMOXINE HYDROCHLORIDE:Prophylaxis:0.31), (BACITRACINNEOMYCIN SULFATEPOLYMYXIN B SULFATE:Prophylaxis:0.74), (BACITRACINNEOMYCINPOLYMYXIN B:Prophylaxis:0.31), (BACLOFENCYCLOBENZAPRINEDICLOFENACGABAPENTINTE TRACAINE:Prophylaxis:0.99), (BALSAM PERU OILCASTOR OILTRYPSIN:Prophylaxis:1.4), (BALSAM PERUCASTOR OILTRYPSIN:Prophylaxis:0.79), (BAMBUTEROL:Prophylaxis:0.33), (BARIUM:Prophylaxis:00.62), (BELLADONNAERGOTAMINE TARTRATEPHENOBARBITAL:Prophylaxis:0.51), (BENFLUOREX:Prophylaxis:0.05), (BEN PERIDOL:Prophylaxis:0.42), (BENRALIZUMAB:Prophylaxis:0.77), (BENZOCAINEMENTH OL:Prophylaxis:0.13), (BENZOCAINERESORCINOL:Prophylaxis:0.54), (BENZOYL PEROXIDEERYTHROMYCIN:Prophylaxis:0.5), (BENZPHETAMINE HYDROCHLORIDE:Prophylaxis:0.15), (BEPOTASTINEBEPOTASTINE BESILATE:Prophylaxis:0.72), (BEXAROTENE:Prophylaxis:1.56), (BIFIDUM:Prophylaxis:1.01), (BISMUTH SUBCITRATE:Prophylaxis:0.51), (BISMUTH SUBSALICYLATEGLYCERINPROPYLENE GLYCOL:Prophylaxis:0.8), (BISMUTH SUBSALICYLATEMETRONIDAZOLETETRACYCLINE HYDROCHLORIDE:Prophylaxis:0.3), (BORNAPRINE HYDROCHLORIDE:Prophylaxis:0.67), (BOTULINUM TOXIN NOS:Prophylaxis:0.16), (BOTULISM ANTITOXIN:Prophylaxis:1.33), (BRIMONIDINE TARTRATEDORZOLAMIDE HYDROCHLORIDETIMOLOL MALEATE:Prophylaxis:0.64), (BRIVUDINE:Prophylaxis:0.84), (BRODALUMAB:Prophylaxis: 0.91), (BROMPHENIRAMINE:Prophylaxis:0.21), (BROMPHENIRAMINE MALEATEDEXTROMETHORPHAN HYDROBROMIDEPSEUDOEPHEDRINE HYDROCHLORIDE:Prophylaxis:0.39), (BROMPHENIRAMINE MALEATEPSEUDOEPHEDRINE HYDROCHLORIDE:Prophylaxis:0.26), (BROMPHENIRAMINEPSEUDOEPHEDRINE:Proph ylaxis:0.44), (BUCLIZINE HYDROCHLORIDE:Prophylaxis:0.2), (BUDESONIDEINDACATEROL MALEATE:Prophylaxis:0.66), (BUFLOMEDIL:Prophylaxis:0.7), (BUPIVACAINE HYDROCHLORIDEEPINEPHRINE BITARTRATE:Prophylaxis:0.71), (BUPRANOLOL:Prophylaxis:0.09), (BUPROPION HYDROBROMIDE:Prophylaxis:0.63), (CABOTEGRAVIR:Prophylaxis:0.28), (CAFFEINEER GOTAMINE TARTRATE:Prophylaxis:0.7), (CAFFEINEERGOTAMINEERGOTAMINE TARTRATE:Prophylaxis:0.21), (CAFFEINEINDOMETHACINPROCHLORPERAZINE MALEATE:Prophylaxis:0), (CAFFEINEISOMETHEPTENEMETHIMAZOLE:Prophylaxis:1.13), (CAFFEINEMAGNESIUM SALICYLATE:Prophylaxis:0.21), (CAJUPUT OILEUCALYPTUS OILPEPPERMINT OIL:Prophylaxis:0.97), (CALAMINE LOTION:Prophylaxis:0.76), (CALCIUM BICARBONATE:Prophylaxis:1.42), (CALCIUM CARBIMIDE:Prophylaxis:0.33), (CALCIUM CARBONATEDIMETHICONE:Prophylaxis:0.28), (CALCIUM CARBONATEMAGNESIUM CARBONATE:Prophylaxis:0.25), (CALCIUM CARBONATE\SODIUM ALGINATE\SODIUM BICARBONATE:Prophylaxis:0.43), (CALCIUM CHLORIDEDEXTROSE MONOHYDRATEHYDROCHLORIC ACIDMAGNESIUM CHLORIDE\SODIUM BICARBONATE\SODIUM CHLORIDE\SODIUM HYDROXIDE\SODIUM LACTATE:Prophylaxis:0.94), (CALCIUM CHLORIDEDEXTROSE MONOHYDRATEMAGNESIUM CHLORIDE\SODIUM BICARBONATE\SODIUM CHLORIDE\SODIUM LACTATE:Prophylaxis:0.67), (CALCIUM CHLORIDEDEXTROSE MONOHYDRATEMAGNESIUM CHLORIDE\SODIUM CHLORIDE\SODIUM LACTATE:Prophylaxis:0.8), (CALCIUM CHLORIDEMAGNESIUM CHLORIDE\POTASSIUM CHLORIDE\SODIUM BICARBONATE\SODIUM CHLORIDE\SODIUM PHOSPHATE, DIBASIC:Prophylaxis:0.76), (CALCIUM CHLORIDE\POTASSIUM CHLORIDE\SODIUM LACTATE:Prophylaxis:0.01), (CALCIUM CLDEXTROSEMAGNESIUM CLSODIUM BICARSODIUM CLSODIUM LACT:Prophylaxis:0.54), (CALCIUM MONOETHYLFUMARATEDIMETHYL FUMARATEMAGNESIUM MONOETHYLFUMARATE\ZINC MONOETHYLFUMARATE:Prophylaxis:0.1), (CALCIUM PANTOTHENATECUPRIC SULFATECYANOCOBALAMINFERROUS ASPARTO GLYCINATEFERROUS FUMARATEFOLIC ACIDMAGNESIUM SULFATENIACINAMIDEPYRIDOXINE HYDROCHLORIDE\RIBOFLAVIN\SODIUM ASCORBATETHIAMINE MONONITRATE\ZINC SULFATE:Prophylaxis:0.76), (CALCIUM PANTOTHENATECYANOCOBALAMINNIACINAMIDEPYRIDOXINE HYDROCHLORIDE\RIBOFLAVINTHIAMINE HYDROCHLORIDE:Prophylaxis:0.81), (CALCIUM PANTOTHENATENIACINAMIDEPYRIDOXINE HYDROCHLORIDE\RIBOFLAVINTHIAMINE HYDROCHLORIDE:Prophylaxis:0.59), (CALCIUM PANTOTHENATENIACINAMIDEPYRIDOXINE HYDROCHLORIDE\RIBOFLAVINTHIAMINE MONONITRATE:Prophylaxis:1.01), (CALCIUM SULFATE:Prophylaxis:0.74), (CALCIUM SULFATEHERBALS:Prophylaxis:0.78), (CALCIUMCHOLECALCIFEROLMAGNESIUMZ INC:Prophylaxis:0.46), (CALCIUMERGOCALCIFEROL:Prophylaxis:0.83), (CAMOSTAT:Pro phylaxis:0.78), (CAMPHOR (SYNTHETIC)MENTHOLMETHYL SALICYLATE:Prophylaxis:0.94), (CAMPHORATED PHENOL:Prophylaxis:0.15), (CANDESARTAN CILEXETILFELODIPINE:Prophylaxis:0.54), ( ALBICANS:Prophylaxis:0.17), (CANGRELOR:Prophylaxis:0.55), (CAPECITABINEOXALIPL ATIN:Prophylaxis:0.91), (CAPSICUM:Prophylaxis:0.48), (CAPSICUM OLEORESIN:Prophylaxis:0.46), (CAPTOPRILHYDROCHLOROTHIAZIDE:Prophylaxis:0.74), (CARBIDOPAMELEVODOPA:Prophylaxis:0.46), (CARBINOXAMINE:Prophylaxis:0.19), (CARBINOXAMINE MALEATE:Prophylaxis:0.71), (CARBOMER HOMOPOLYMER TYPE C (ALLYL PENTAERYTHRITOL CROSSLINKED):Prophylaxis:0.49), (CARBON DIOXIDE:Prophylaxis:0.9), (CARBOXYMETHYLCELLULOSE SODIUMGELATINPECTIN:Prophylaxis:0.6), (CARGLUMIC ACID:Prophylaxis:0.66), (CARVEDILOL HYDROCHLORIDE:Prophylaxis:0.3), (CAT'S CLAW:Prophylaxis:0.5), (CEDIRANIB:Prophylaxis:1.08), (CEFATRIZINE:Prophylaxis:0.38), (CEFTOBIPROLE:Prophylaxis:0.44), (CENTELLA ASIATICA:Prophylaxis:0.42), (CEP-9722:Prophylaxis:0.34), (CEPHALEXIN ANHYDROUS:Prophylaxis:0.4), (CEPHALEXIN HYDROCHLORIDE:Prophylaxis:0.36), (CEPHALEXIN MONOHYDRATE:Prophylaxis:0.56), (CERIVASTATIN:Prophylaxis:0.05), (CERIVASTATIN SODIUM:Prophylaxis:1.37), (CERTOLIZUMAB:Prophylaxis:1.53), (CETRORELIX:Prophylaxi s:0.02), (CETRORELIX ACETATE:Prophylaxis:0.54), (CHAMOMILE:Prophylaxis:0.28), (CHENODIOL:Prophylaxis:0.32), (CHLOPHEDIANOL HYDROCHLORIDEDEXBROMPHENIRAMINE MALEATEPHENYLEPHRINE HYDROCHLORIDE:Prophylaxis:0.18), (CHLORAMPHENICOL PALMITATE:Prophylaxis:0.17), (CHLORAMPHENICOLPREDNISOLONE:Prophylaxis:0.51), (CHLORDIAZEPOXIDECLIDINIUM:Prophylaxis:1), (VULGARIS:Prophylaxis:0.33), (CHLORHEXIDINE GLUCONATECHLOROCRESOLHEXAMIDINE DIISETHIONATE:Prophylaxis:0.29), (CHLORIDE ION:Prophylaxis:0.88), (CHLORINE:Prophylaxis:0.46), (CHLORMEZANONE:Prophylaxis:0.45), (CHLORODEHYDROMETHYLTESTOSTERONE:Prophylaxis:0.44), (CHLOROPROCAINE: Prophylaxis:0.07), (CHLORPHENIRAMINE MALEATEHYDROCODONE BITARTRATEPSEUDOEPHEDRINE HYDROCHLORIDE:Prophylaxis:0.59), (CHLORPHENIRAMINE MALEATEIBUPROFENPHENYLEPHRINE HYDROCHLORIDE:Prophylaxis:0.01), (CHLORPHENIRAMINE MALEATEPHENYLEPHRINE HYDROCHLORIDE:Prophylaxis:0.43), (CHLORPHENIRAMINE MALEATEPSEUDOEPHEDRINE HYDROCHLORIDE:Prophylaxis:0.74), (CHLORPROMAZINE HIBENZATE:Prophylaxis:0.49), (CHOLECALCIFEROLCYANOCOBALAMIN CO-57FOLIC ACIDPYRIDOXINE HYDROCHLORIDE:Prophylaxis:0.27), (CHOLECALCIFEROLGENISTEINZINC GLYCINATE:Prophylaxis:0.75), (CHOLIC ACID:Prophylaxis:1.16), (CHOLINE BITARTRATE:Prophylaxis:0.22), (CHOLINE SALICYLATE:Prophylaxis:0.27), (CHOLINETRAZODONE HYDROCHLORIDE:Prophylaxis:0.81), (CHONDROITIN SULFATE (BOVINE)HYALURONATE SODIUM:Prophylaxis:0.01), (CHROMIC CHLORIDECUPRIC CHLORIDEMANGANESE CHLORIDE\ZINC CHLORIDE:Prophylaxis:0.25), (CICLOPIROX OLAMINE:Prophylaxis:1.6), (CILAZAPRIL ANHYDROUSHYDROCHLOROTHIAZIDE:Prophylaxis:0.62), (CILENGITIDE:Prophylaxis: 0.4), (CINNAMON OIL:Prophylaxis:0.42), (CIPROFLOXACIN HYDROCHLORIDEHYDROCORTISONE:Prophylaxis:0.4), (CIPROFLOXACINDEXAMET HASONE:Prophylaxis:0.88), (CIPROFLOXACINFLUOCINOLONE ACETONIDE:Prophylaxis:0.15), (CITALOPRAM HYDROCHLORIDE:Prophylaxis:0.55), (CITRIC ACID MONOHYDRATEDEXTROSE\SODIUM CITRATE\SODIUM PHOSPHATE, MONOBASIC:Prophylaxis:0.53), (CITRIC ACID MONOHYDRATETRISODIUM CITRATE DIHYDRATE:Prophylaxis:0.9), (CIXUTUMUMAB:Prophylaxis:0.85), (CLARITHROMYCIN LACTOBIONATE:Prophylaxis:0.65), (CLENBUTEROL:Prophylaxis:0.2), (CLEVIDIPINE:Prop hylaxis:0.74), (CLIDINIUM BROMIDE:Prophylaxis:0.24), (CLINDAMYCIN PALMITATE HYDROCHLORIDE:Prophylaxis:0.47), (CLINDAMYCIN PHOSPHATETRETINOIN:Prophylaxis:0.34), (CLIOQUINOL:Prophylaxis:0.44), (CLOBETAS ONE BUTYRATENYSTATINOXYTETRACYCLINE CALCIUM:Prophylaxis:0.7), (CLOCORTOLONE PIVALATE:Prophylaxis:0.88), (CLONIXINCYCLOBENZAPRINE:Prophylaxis:0.63), (CLOPI DOGREL HYDROBROMIDE:Prophylaxis:0.26), (CLOPREDNOL:Prophylaxis:0.44), (TETANICLOSTRIDIUM TETANI TOXOID ANTIGEN (FORMALDEHYDE INACTIVATED)DIPHTHERIAE TOXOID ANTIGEN (FORMALDEHYDE INACTIVATED):Prophylaxis:1.14), (CLOTHIAPINE:Prophylaxis:0.27), (COAGULATION FACTOR IX HUMANCOAGULATION FACTOR VII HUMANCOAGULATION FACTOR X HUMANPROTHROMBIN:Prophylaxis:0.52), (COAGULATION FACTOR VII HUMAN:Prophylaxis:1), (CODEINE HYDROCHLORIDE:Prophylaxis:0.68), (CODEINE PHOSPHATEGUAIFENESINPSEUDOEPHEDRINE HYDROCHLORIDE:Prophylaxis:0.79), (CODEINE PHOSPHATEPROMETHAZINE:Prophylaxis:0.99), (CODEINESISYMBRIUM OFFICINALE WHOLE:Prophylaxis:0.84), (COLCHICINEOPIUMTIEMONIUM METHYLSULFATE:Prophylaxis:0.9), (COLCHICINEPROBENECID:Prophylaxis:0.94), (COL ISTIN A SODIUM METHANESULFONATE:Prophylaxis:1.19), (COPPER OR LEVONORGESTREL:Prophylaxis:0.41), (COPPERINTRAUTERINE DEVICE:Prophylaxis:0.21), (COUMARIN:Prophylaxis:1.02), (CRANBERRY CONCENTRATE:Prophylaxis:0.97), (CRATAEGUS LAEVIGATA FRUIT:Prophylaxis:0.56), (CREATINE MONOHYDRATE:Prophylaxis:0.23), (CROFELEMER:Prophylaxis:0.53), (CROTALIDAE POLYVALENT IMMUNE FAB (OVINE):Prophylaxis:1.51), (CUMIN:Prophylaxis:0.6), (CUPRIC CHLORIDEMANGANESE CHLORIDE\POTASSIUM IODIDE\SODIUM FLUORIDE\SODIUM SELENITE\ZINC CHLORIDE:Prophylaxis:0.08), (CURCUMINDIETARY SUPPLEMENT:Prophylaxis:0.51), (CYANAMIDE:Prophylaxis:1.2), (CYANOCOBALAMIN CO-57LEVOMEFOLATE CALCIUMPYRIDOXINE HYDROCHLORIDE\RIBOFLAVIN:Prophylaxis:0.78), (CYANOCOBALAMINPYRIDOXINE HYDROCHLORIDE\RIBOFLAVIN\SODIUM PANTOTHENATETHIAMINE HYDROCHLORIDEVITAMIN B COMPLEX:Prophylaxis:0.87), (CYCLOPHOSPHAMIDEDOXORUBICINPREDNISONERIT UXIMABVINCRISTINE:Prophylaxis:1.52), (CYSTEAMINE:Prophylaxis:0.58), (CYSTEAMIN E BITARTRATE:Prophylaxis:0.62), (CYSTEAMINE HYDROCHLORIDE:Prophylaxis:0.62), (CYSTINE:Prophylaxis:0.89), (CYTARABINE OCFOSFATE:Prophylaxis:1.64), (CYTARABINEDAUNORUBICIN:Prophylaxis:0.94), (DABR AFENIBTRAMETINIB:Prophylaxis:0.52), (DACETUZUMAB:Prophylaxis:0.4), (DACOMITIN 1B:Prophylaxis:0.92), (DALBAVANCIN:Prophylaxis:0.23), (DALBAVANCIN HYDROCHLORIDE:Prophylaxis:0.05), (DALOTUZUMAB:Prophylaxis:0.7), (DAPSONEFER ROUS OXALATE:Prophylaxis:1.07), (DARIFENACIN:Prophylaxis:1.15), (DARUNAVIRRITONAVI R:Prophylaxis:0.46), (DASABUVIR SODIUM MONOHYDRATE:Prophylaxis:0.71), (DASABUVIR SODIUM MONOHYDRATEOMBITASVIR HEMINONAHYDRATEPARITAPREVIR DIHYDRATERITONAVIR:Prophylaxis:1.03), (DASABUVIROMBITASVIRPARITAPREVI RRIBAVIRINRITONAVIR:Prophylaxis:0.36), (DAUNORUBICIN CITRATE:Prophylaxis:0.86), (DELAPRIL HYDROCHLORIDEINDAPAMIDE:Prophylaxis:0.39), (DELAVIRDINE:Prophylaxis:0.06), (D EOXYRIBONUCLIEC ACID:Prophylaxis:1.08), (DESLORATADINEPSEUDOEPHEDRINE SULFATE:Prophylaxis:0.65), (DEUTETRABENAZINE:Prophylaxis:0.45), (DEXPANTHENOL NIACINAMIDEPYRIDOXINE HYDROCHLORIDE\RIBOFLAVINTHIAMINE HYDROCHLORIDE:Prophylaxis:0.4), (DEXPANTHENOLXYLOMETAZOLINE HYDROCHLORIDE:Prophylaxis:0.49), (DEXTRAN 70GLYCERINHYPROMELLOSES:Prophylaxis:0.58), (DEXTRAN SULFATE SODIUM SALT:Prophylaxis:1.31), (DEXTRANHYPROMELLOSES:Prophylaxis:0.5), (DEXTROMETH ORPHAN HYDROBROMIDEGUAIFENESINPHENYLEPHRINE HYDROCHLORIDE:Prophylaxis:0.34), (DEXTROMETHORPHAN HYDROBROMIDEGUAIFENESINPSEUDOEPHEDRINE HYDROCHLORIDE:Prophylaxis:0.72), (DEXTROMETHORPHAN HYDROBROMIDEPROMETHAZINE HYDROCHLORIDE:Prophylaxis:0.78), (DEXTROSE MONOHYDRATEDEXTROSE MONOHYDRATEDOBUTAMINE HYDROCHLORIDEDOBUTAMINE HYDROCHLORIDE:Prophylaxis:0.6), (DEXTROSEFRUCTOSEPHOSPHORIC ACID:Prophylaxis:0.14), (DEXTROSEHEPARIN:Prophylaxis:1.05), (DEXTROSELIDOCAIN E HYDROCHLORIDE:Prophylaxis:0.1), (DIACETYL BENZOYL LATHYROL:Prophylaxis:0.57), (DIACETYLMORPHINE HYDROCHLORIDE:Prophylaxis:0.37), (DIATRIZOATE MEGLUMINE:Prophylaxis:0.7), (DIBUCAINE:Prophylaxis:0.52), (DICHLORPHENAMIDE:Pr ophylaxis:0.64), (DIENOGEST:Prophylaxis:1.55), (DIETARY SUPPLEMENTFLAVOCOXID:Prophylaxis:0.75), (DIETHYLAMINE SALICYLATE:Prophylaxis:0.61), (DIETHYLPROPION:Prophylaxis:0.47), (DIHYDROERGOC RISTINE:Prophylaxis:0.53), (DILOXANIDE FUROATETINIDAZOLE:Prophylaxis:0.37), (DIPHENHYDRAMINE HYDROCHLORIDEPHENYLEPHRINE HYDROCHLORIDE:Prophylaxis:0.66), (DIPHENHYDRAMINE HYDROCHLORIDE\ZINC ACETATE:Prophylaxis:0.37), (DIPHENOXYLATE HYDROCHLORIDE:Prophylaxis:0.07), (DISODIUM FUMARATE:Prophylaxis:0.37), (DOLASETRON:Prophylaxis:0.2), (DONEPEZIL HYDROCHLORIDEMEMANTINE HYDROCHLORIDE:Prophylaxis:0.48), (DOTHIEPINDOTHIEPIN HYDROCHLORIDE:Prophylaxis:0.07), (DOXERCALCIFEROL:Prophylaxis:1.11), (DOXIFLU RIDINE:Prophylaxis:0.62), (DOXYCYCLINE CALCIUM:Prophylaxis:0.36), (DROSPIRENONE:Prophylaxis:0.76), (DYCLONINE:Prophylaxi s:0.02), (DYCLONINE HYDROCHLORIDE:Prophylaxis:0.99), (PURPUREA:Prophylaxis:0.61), (EFAVIRENZLAMIVUDINEZIDOVUDINE:Prophylaxis:0.14), (EFLORNITHINE:Prophylaxis:0.43), (EFLORNITHINE HYDROCHLORIDE:Prophylaxis:0.57), (ELECTROLYTES NOSMINERALSSORBITOL:Prophylaxis:0.1), (ELETRIPTAN:Prophylaxis:0.45), (ELM:Proph ylaxis:0.14), (EMACTUZUMAB:Prophylaxis:0.78), (EMTRICITABINERILPIVIRINE HYDROCHLORIDETENOFOVIR ALAFENAMIDE FUMARATE:Prophylaxis:0.24), (EMTRICITABINETENOFOVIRZIDOVUDINE:Prophylaxis: 0.7), (ENBUCRILATE:Prophylaxis:0.91), (ENTOSPLETINIB:Prophylaxis:0.68), (EPACADOST AT:Prophylaxis:1.5), (EPHEDRA:Prophylaxis:0.44), (EPHEDRINE HYDROCHLORIDEGUAIFENESIN:Prophylaxis:0.47), (EPHEDRINE SULFATE:Prophylaxis:0.33), (EPROSARTAN MESYLATEHYDROCHLOROTHIAZIDE:Prophylaxis:0.35), (EQUISETUM ARVENSE TOP:Prophylaxis:0.03), (ERGOCALCIFEROLPHYTONADIONETOCOPHEROLVITAMIN A PALMITATE:Prophylaxis:0.14), (ERGONOVINE MALEATE:Prophylaxis:0.28), (ERGOTAMINE TARTRATE:Prophylaxis:0.26), (ERYTHROMYCIN ESTOLATE:Prophylaxis:0.25), (ERYTHROMYCIN STEARATE:Prophylaxis:0.31), (ESKETAMINE:Prophylaxis:0.91), (ESLICARBAZEPINE:Prop hylaxis:0.4), (ESSENTIAL OILS:Prophylaxis:0.34), (ESTRADIOL BENZOATE:Prophylaxis:0.74), (ESTRADIOL HEMIHYDRATE:Prophylaxis:0.98), (ESTRADIOL HEMIHYDRATENORETHINDRONE ACETATE:Prophylaxis:0.07), (ESTRADIOL VALERATENORETHINDRONE:Prophylaxis:0.12), (ESTRADIOLESTRIOLESTRONE:Pro phylaxis:0.17), (ESTRADIOLPROGESTERONE:Prophylaxis:0.79), (ESTROGENS, CONJUGATEDNORGESTREL:Prophylaxis:0.15), (ESTROGENSMETHYLTESTOSTERON E:Prophylaxis:0.94), (ESTROGENSPROGESTERONE:Prophylaxis:0.16), (ESTRONE:Prophyla xis:0.51), (ETEPLIRSEN:Prophylaxis:0.79), (ETHAMBUTOL HYDROCHLORIDEISONIAZIDPYRAZINAMIDERIFAMPIN:Prophylaxis:0.97), (ETHAM BUTOLISONIAZIDPYRAZINAMIDERIFAMYCIN:Prophylaxis:1.18), (ETHINYL ESTRADIOLETHYNODIOL DIACETATE:Prophylaxis:0.87), (ETHINYL ESTRADIOLFERROUS FUMARATENORETHINDRONE:Prophylaxis:0.84), (ETHINYL ESTRADIOLFERROUS FUMARATENORETHINDRONE ACETATE:Prophylaxis:0.63), (ETHYLENE GLYCOL:Prophylaxis:0.07), (ETHYLPHENIDATE:Prophylaxis:0.51), (ETILEFRINE:Prophyla xis:0.28), (EUCALYPTUS OIL:Prophylaxis:0.39), (FACTOR IX COMPLEX:Prophylaxis:0.57), (FARLETUZUMAB:Prophylaxis:0.43), (FECAL MICROBIOTA:Prophylaxis:0.77), (FENOPROFEN:Prophylaxis:0.83), (FENOPROFEN CALCIUM:Prophylaxis:0.99), (FENRETINIDE:Prophylaxis:0.96), (FENSPIRIDE HYDROCHLORIDE:Prophylaxis:0.9), (FENTANYL HYDROCHLORIDE:Prophylaxis:0.9), (FERRIC POLYSACCHARIDE COMPLEX:Prophylaxis:0.89), (FERROUS ASPARTO GLYCINATEFERROUS FUMARATE:Prophylaxis:0.28), (FERROUS FUMARATEMESTRANOLNORETHINDRONE:Prophylaxis:0.32), (FERUMOXYTOL:Proph ylaxis:0.77), (FEVERFEW:Prophylaxis:0.11), (FEXOFENADINE HYDROCHLORIDEPSEUDOEPHEDRINE HYDROCHLORIDE:Prophylaxis:0.48), (FIG FRUIT OILSORBITOL:Prophylaxis:0.67), (FLIBANSERIN:Prophylaxis:0.06), (FLORBETAPIR F-18:Prophylaxis:0.56), (FLOXURIDINE:Prophylaxis:0.8), (FLUDEOXYGLUCOSE F-18:Prophylaxis:0.9), (FLUOCORTOLONE:Prophylaxis:0.31), (FLUOROURACILIRINOTECA NLEUCOVORINOXALIPLATIN:Prophylaxis:0.73), (FLUOXETINE HYDROCHLORIDEOLANZAPINE:Prophylaxis:0.71), (FLUOXYMESTERONE:Prophylaxis: 0.43), (FLUPENTIXOL DECANOATE:Prophylaxis:0.29), (FLUPENTIXOL DIHYDROCHLORIDE:Prophylaxis:0.39), (FLUPIRTINE MALEATE:Prophylaxis:0.05), (FLUSPIRILENE:Prophylaxis:0.02), (FOLIC ACIDIRON:Prophylaxis:0.58), (FOLLITROPINLUTEINIZING HORMONE:Prophylaxis:0.02), (FORMALDEHYDE:Prophylaxis:0.46), (FOSAMPRENAVIRR ITONAVIR:Prophylaxis:0.27), (FOSBRETABULIN TROMETHAMINE:Prophylaxis:1.25), (FROVATRIPTAN:Prophylaxis:0.04), (FURAZOLIDON E:Prophylaxis:0.52), (FUROSEMIDE SODIUMSPIRONOLACTONE:Prophylaxis:1.02), (FURSULTIAMINE:Prophylaxis:1.83), (GA DOTERIDOL:Prophylaxis:0.06), (GANITUMAB:Prophylaxis:0.69), (GEMIFLOXACIN MESYLATE:Prophylaxis:0.34), (GENISTEIN:Prophylaxis:0.31), (GENTIAN VIOLET:Prophylaxis:1.12), (GIMERACIL:Prophylaxis:0.66), (GINKGO BILOBA LEAF EXTRACT:Prophylaxis:0.82), (GINSENG EXTRACT:Prophylaxis:0.36), (GLATIRAMER:Prophylaxis:1.02), (GLECAPREVIRPIBRENT ASVIR:Prophylaxis:0.5), (GLIMEPIRIDEPIOGLITAZONE HYDROCHLORIDE:Prophylaxis:0.62), (GLIMEPIRIDEROSIGLITAZONE MALEATE:Prophylaxis:0.83), (GLIQUIDONE:Prophylaxis:0.93), (GLUCONATE:Prophylaxis:00.89), (GLYCERINPETROLATUMPHENYLEPHRINE HYDROCHLORIDEPRAMOXINE HYDROCHLORIDE:Prophylaxis:0.38), (GLYCEROL PHENYLBUTYRATE:Prophylaxis:0.15), (GOLD:Prophylaxis:0.43), (GOLDENSEAL:Prophyla xis:0.27), (GOTU KOLA:Prophylaxis:0.02), (GRAPE SEED EXTRACT:Prophylaxis:0.53), (GRAPEFRUIT SEED EXTRACT:Prophylaxis:0.26), (GRISEOFULVIN:Prophylaxis:0.95), (GUAIFENESINPHENYL EPHRINE HYDROCHLORIDE:Prophylaxis:1.1), (GUSELKUMAB:Prophylaxis:0.57), (HALCINONIDE:P rophylaxis:0.9), (HAWTHORN LEAF WITH FLOWER:Prophylaxis:0.51), (HEPARIN SODIUMSODIUM CHLORIDE:Prophylaxis:0.51), (HEPTAMINOL:Prophylaxis:0.63), (HEPTAMINOL HYDROCHLORIDE:Prophylaxis:0.48), (HERBALSHOMEOPATHICSGOLDENSEAL:Proph ylaxis:0.39), (HERBALSVITIS VINIFERA SEED:Prophylaxis:0.31), (HEXACHLOROPHENE:Prophylaxis:0.54), (HEXAMIDINE DIISETHIONATE:Prophylaxis:0.23), (HISTAMINE:Prophylaxis:0.04), (HOMATROPINE HYDROBROMIDE:Prophylaxis:0.53), (HOMEOPATHICSZINC ACETATE\ZINC GLUCONATE:Prophylaxis:0.15), (HOMOSALATEOCTINOXATEOCTOCRYLENE\ZINC OXIDE:Prophylaxis:0.19), (HUMAN CLOSTRIDIUM TETANI TOXOID IMMUNE GLOBULIN:Prophylaxis:0.5), (HUMAN COLOSTRUM:Prophylaxis:0.4), (HYALURONIC ACIDLIDOCAINE HYDROCHLORIDE:Prophylaxis:0.46), (HYALURONIDASE (HUMAN RECOMBINANT):Prophylaxis:0.02), (HYDROCHLORIC ACID:Prophylaxis:0.57), (HYDROCHLOROTHIAZIDEMETOPROLOL:Prophylaxis:0.71), (H YDROCHLOROTHIAZIDEMETOPROLOL TARTRATE:Prophylaxis:1.1), (HYDROCHLOROTHIAZIDEMOEXIPRIL HYDROCHLORIDE:Prophylaxis:0.86), (HYDROCHLOROTHIAZIDEPROPRANOLOL HYDROCHLORIDE:Prophylaxis:0.75), (HYDROCHLOROTHIAZIDEZOFENOPRIL:Prophyl axis:0.55), (HYDROCODONE BITARTRATEPHENYLPROPANOLAMINE HYDROCHLORIDE:Prophylaxis:0.63), (HYDROCODONEHYDROCODONE BITARTRATEIBUPROFEN:Prophylaxis:0.19), (HYDROCODONEIBUPROFEN:Prophylaxis: 0.51), (HYDROCORTISONEMICONAZOLE:Prophylaxis:0.37), (HYDROCORTISONEPRA MOXINE:Prophylaxis:0.52), (HYDROXYPROPYL CELLULOSE:Prophylaxis:1.08), (HYOSCYAMINE SULFATEMETHENAMINEMETHYLENE BLUE\SODIUM PHOSPHATE, MONOBASIC:Prophylaxis:0.42), (HYPERICUM PERFORATUM:Prophylaxis:0.04), (HYPOCHLORITE ION:Prophylaxis:0.11), (IBUPROFEN LYSINE:Prophylaxis:0.7), (IDEBENONE:Prophylaxis:0.51), (ILOPERIDONE:Prophylaxis:0.35), (ILOPROST TROMETHAMINE:Prophylaxis:0.36), (IMIGLUCERASE:Prophylaxis:2.09), (IMIPRAMINE PAMOATE:Prophylaxis:0.39), (INDAPAMIDE HEMIHYDRATE:Prophylaxis:0.13), (INDIUM IN-111 CHLORIDEINDIUM IN-111 PENTETREOTIDEPENTETREOTIDE:Prophylaxis:0.12), (INDOBUFEN:Prophylaxis:0.5), (IN DOCYANINE GREEN:Prophylaxis:0.19), (INFLUENZA A VIRUS A/CALIFORNIA/7/2009 X-179A (H1N1) ANTIGEN (FORMALDEHYDE INACTIVATED)\INFLUENZA A VIRUS A/TEXAS/50/2012 X-223A (H3N2) ANTIGEN (FORMALDEHYDE INACTIVATED)\INFLUENZA B VIRUS B/BRISBANE/60/2008 ANTIGEN (FORMALDEHYDE INACTIVATED)\INFLUENZA B VIRUS B/MASSACHUSETTS/2/2012 ANTIGEN (FORMALDE:Prophylaxis:0.76), (INIPARIB:Prophylaxis:0.61), (INOSINE PRANOBEX:Prophylaxis:0.88), (INTERFERON ALFACON-1:Prophylaxis:1.14), (INTERFERON GAMMA-1B:Prophylaxis:2.37), (INULIN:Prophylaxis:0.65), (IODINE POVACRYLEXISOPROPYL ALCOHOL:Prophylaxis:0.35), (IODINETOSITUMOMAB:Prophylaxis:0.65), (IOTHALAMIC ACID:Prophylaxis:0.59), (IOXAGLATE MEGLUMINEIOXAGLATE SODIUM:Prophylaxis:0.35), (ISOCARBOXAZID:Prophylaxis:0.46), (ISOFLAVONES SOY:Prophylaxis:0.98), (ISOLEUCINE:Prophylaxis:0.39), (ISOMETHEPTENE:Prophylaxis:0.14), (ISONIAZIDPYRIDOXINE:Prophylaxis:0.98), (ISOPROPYL ALCOHOL:Prophylaxis:0.91), (ISOPROPYL ALCOHOLTENECTEPLASE:Prophylaxis:0.52), (ISOPROPYL MYRISTATE:Prophylaxis:0.41), (ISOSULFAN BLUE:Prophylaxis:0.03), (JOSAMYCIN:Prophylaxis:0.94), (JWH-018:Prophylaxis:0.24), (KAOLIN:Prophylaxis:1.06), (KERATIN:Prophylaxis:0.2), (KETANSERI N:Prophylaxis:0.77), (KETOBEMIDONE:Prophylaxis:0.02), (KETOBEMIDONE HYDROCHLORIDE:Prophylaxis:0.4), (REUTERI:Prophylaxis:0.89), (LACTOPEROXIDASE BOVINELYSOZYME HYDROCHLORIDE:Prophylaxis:0.59), (LACTULOSEPARAFFIN:Prophylaxis:0.72), (LAMIV UDINENEVIRAPINEZIDOVUDINE:Prophylaxis:0.32), (LAMIVUDINETENOFOVIR DISOPROXIL FUMARATE:Prophylaxis:0.13), (LANINAMIVIR:Prophylaxis:1.07), (LANOLINMINERAL OIL:Prophylaxis:0.31), (LAVENDER OIL:Prophylaxis:0.52), (LESINURAD:Prophylaxis:0.73), (LESTAURTINIB:Prophylaxis:1.25), (LETROZOLERIBOCICLIB:Prophylaxis:0.72), (LEUCINE:Prophylaxis:0.66), (LEUPROLIDE ACETATENORETHINDRONE ACETATE:Prophylaxis:0.26), (LEVAMISOLE:Prophylaxis:0.06), (LEVODOPAUNSPECIFIE D INGREDIENT:Prophylaxis:0.43), (LEVOLEUCOVORIN DISODIUM:Prophylaxis:0.26), (LEVOMEFOLATE CALCIUM\METHYLCOBALAMINPYRIDOXAL 5-PHOSPHATE:Prophylaxis:0.41), (LEVOMEFOLIC ACIDMETHYLCOBALAMINTURMERIC:Prophylaxis:0.35), (LEVOMEPROMAZINE HYDROCHLORIDELEVOMEPROMAZINE MALEATE:Prophylaxis:0.79), (LEVOMETHADONE:Prophylaxis:0.41), (LEVORPHANOL:Pro phylaxis:0.35), (LEVORPHANOL TARTRATE:Prophylaxis:0.66), (LIDOCAINE HYDROCHLORIDEMETHYLPREDNISOLONE ACETATE:Prophylaxis:0.68), (LIGHT MINERAL OILMINERAL OIL:Prophylaxis:0.3), (LINCOMYCIN:Prophylaxis:0.79), (LINOLEIC ACID:Prophylaxis:0.39), (LIRILUMAB:Prophylaxis:0.73), (LITHIUM CITRATE:Prophylaxis:0.14), (LITHIUM OROTATE:Prophylaxis:0.17), (LITHIUM SULFATE:Prophylaxis:0.39), (LOBAPLATIN:Prophylaxis:0.63), (LOVASTATINNIACIN:Prop hylaxis:0.82), (LUMACAFTOR:Prophylaxis:1.25), (LUTETIUM LU-177:Prophylaxis:1.34), (LYMPHOCYTES:Prophylaxis:0.4), (MAGNESIUM ACETATE TETRAHYDRATE\POTASSIUM ACETATE\SODIUM CHLORIDE:Prophylaxis:0.39), (MAGNESIUM ALUMINUM SILICATE:Prophylaxis:0.4), (MAGNESIUM CHLORIDE\POTASSIUM CHLORIDE\POTASSIUM PHOSPHATE\SODIUM ACETATE\SODIUM CHLORIDE\SODIUM GLUCONATE\SODIUM PHOSPHATE, DIBASIC:Prophylaxis:0.46), (MAGNESIUM LACTATEPYRIDOXINE HYDROCHLORIDE:Prophylaxis:1.21), (MAGNESIUM PHOSPHATE, TRIBASIC, PENTAHYDRATE:Prophylaxis:0.6), (MAGNESIUM STEARATE:Prophylaxis:0.8), (MALATHION:Prophylaxis:0.2), (MALIC ACID:Prophylaxis:0.14), (MALTODEXTRIN:Prophylaxis:0.72), (MANGANESEMANGANES E CHLORIDE:Prophylaxis:0.51), (MARIZOMIB:Prophylaxis:1.06), (RECUTITA:Prophylaxis:0.16), (MECHLORETHAMINE:Prophylaxis:1.44), (MECHLORETHA MINE HYDROCHLORIDE:Prophylaxis:0.62), (MECHLORETHAMINE OXIDE HYDROCHLORIDE:Prophylaxis:0.67), (MECLIZINE MONOHYDROCHLORIDENIACIN:Prophylaxis:1.07), (MECLOFENAMATE SODIUM:Prophylaxis:0.12), (MECLOZINE DIHYDROCHLORIDE:Prophylaxis:0.71), (MELDONIUM:Prophylaxis:0.88), (MENADIONE:P rophylaxis:0.53), (MEPHENESIN:Prophylaxis:0.29), (MEPHENOXALONE:Prophylaxis:0.19), (MEROPENEM ANHYDROUS:Prophylaxis:0.9), (MESTRANOLNORETHINDRONE:Prophylaxis:0.84), (MET APROTERENOL:Prophylaxis:0.03), (METAPROTERENOL SULFATE:Prophylaxis:0.58), (METFORMINSITAGLIPTIN PHOSPHATE:Prophylaxis:0.52), (METFORMINVILDAGLIPTIN:Prophylaxis:0.79), (METHA CHOLINE CHLORIDE:Prophylaxis:0.01), (METHANDROSTENOLONE:Prophylaxis:0.14), (METHENA MINE MANDELATE:Prophylaxis:1.4), (METHOHEXITAL:Prophylaxis:0.36), (METHOHEXITAL SODIUM:Prophylaxis:0.04), (METHOTREXATEMETHOTREXATE SODIUM:Prophylaxis:1.2), (METHSCOPOLAMINEMETHSCOPOLAMINE BROMIDE:Prophylaxis:0.06), (METHSUXIMIDE:Prophylaxis:0.53), (METHYLPREDNISOLO NE OR METHYLPREDNISOLONE ACETATE:Prophylaxis:1.57), (METRONIDAZOLE BENZOATE:Prophylaxis:0.87), (METYROSINE:Prophylaxis:0.29), (MICONAZOLE NITRATE OR TERBINAFINE HYDROCHLORIDE OR TOLNAFTATE:Prophylaxis:0.64), (MICONAZOLE NITRATEPETROLATUM\ZINC OXIDE:Prophylaxis:0.23), (MIFAMURTIDE:Prophylaxis:0.8), (MINERALSPROBIOTICS NOSVITAMINS:Prophylaxis:1.06), (MITOGUAZONE:Prophylaxis:0.46), (MITRAGYNINE:Pr ophylaxis:0.21), (MIVACURIUM CHLORIDE:Prophylaxis:0.32), (MORPHINE\MORPHINE HYDROCHLORIDE\MORPHINE SULFATE\MORPHINE TARTRATE:Prophylaxis:0.73), (NAFARELIN ACETATE:Prophylaxis:0.5), (NAFTIFINE:Prophylaxis:0.45), (NALIDIXIC ACID:Prophylaxis:0.6), (NALOXONE HYDROCHLORIDEPENTAZOCINE HYDROCHLORIDE:Prophylaxis:0.47), (NANDROLONE:Prophylaxis:0.35), (NAPHAZOLINE NAPHAZOLINE HYDROCHLORIDE:Prophylaxis:0.24), (NATTOKINASE:Prophylaxis:0.4), (NATURAL LATEX RUBBER:Prophylaxis:0.57), (NEDOCROMIL SODIUM:Prophylaxis:0.31), (NEFAZODONE:Prophylaxis:0.96), (NEFAZODONE HYDROCHLORIDE:Prophylaxis:1.1), (NEOMYCINPOLYMYXIN:Prophylaxis:0.38), (NERA TINIB:Prophylaxis:0.57), (NESIRITIDE:Prophylaxis:0.63), (NIACINAMIDEPYRIDOXINE HYDROCHLORIDE\RIBOFLAVINTHIAMINE HYDROCHLORIDE:Prophylaxis:0.36), (NIAOULI OILQUININE BENZOATETHIAMINE HYDROCHLORIDE:Prophylaxis:1.19), (NIFURTOINOL:Prophylaxis:1.38), (NILUTAMIDE:Pr ophylaxis:0.5), (NOMEGESTROL:Prophylaxis:0.52), (NOSCAPINE:Prophylaxis:0.45), (OCTIN OXATEOCTISALATE\ZINC OXIDE:Prophylaxis:0.07), (OLODATEROL HYDROCHLORIDE:Prophylaxis:1.43), (OLODATEROLTIOTROPIUM BROMIDE:Prophylaxis:0.93), (OLSALAZINE:Prophylaxis:0.98), (OLSALAZINE SODIUM:Prophylaxis:0.29), (OMBITASVIR:Prophylaxis:0.64), (OMBITASVIR HEMINONAHYDRATEPARITAPREVIR DIHYDRATERITONAVIR:Prophylaxis:0.91), (OMEGA-6 FATTY ACIDS:Prophylaxis:0.68), (OPICAPONE:Prophylaxis:0.81), (OPRELVEKIN:Prophylaxis:0.77), (OREGANO:Prophylaxis:0.26), (ORPHENADRINE HYDROCHLORIDE:Prophylaxis:0.26), (ORTERONEL:Prophylaxis:1), (OSTREA EDULIS SHELL:Prophylaxis:0.81), (OTERACIL:Prophylaxis:0.57), (OTERACIL POTASSIUM:Prophylaxis:0.64), (OXITROPIUM BROMIDE:Prophylaxis:0.4), (OXPRENOLOL:Prophylaxis:0.3), (OXYCODONE TEREPHTHALATE:Prophylaxis:0.23), (OXYMETAZOLINE HYDROCHLORIDEPSEUDOEPHEDRINE HYDROCHLORIDE:Prophylaxis:0.08), (OXYMETHOLONE:Prophylaxis:0.71), (OXYPERTIN E:Prophylaxis:1.04), (PAMABROM:Prophylaxis:0.14), (PANCRELIPASEPANCRELIPASE AMYLASEPANCRELIPASE LIPASEPANCRELIPASE PROTEASE:Prophylaxis:1.42), (PANCRELIPASETYROSINE:Prophylaxis:0.73), (PARALDEH YDE:Prophylaxis:0.44), (PARITAPREVIR:Prophylaxis:0.61), (PAROMOMYCIN:Prophylaxis:0.64), (PARSLEY:Prophylaxis:0.62), (PASCOPYRUM SMITHII POLLEN:Prophylaxis:0.93), (PECTIN:Prophylaxis:0.31), (PEGINTERFERON ALFA-2A OR PEGINTERFERON ALFA-2B OR PEGINTERFERON BETA-1A OR PEGINTERFERON LAMBDA-1A:Prophylaxis:1.07), (PEGINTERFERON ALFA-2BRIBAVIRIN:Prophylaxis:0.68), (PEGINTERFERON LAMBDA-1A:Prophylaxis:1.4), (PEMOLINE:Prophylaxis:0.43), (PENBUTOLOL SULFATE:Prophylaxis:0.35), (PENICILLIN G POTASSIUM:Prophylaxis:0.54), (PENICILLING PROCAINE:Prophylaxis:0.23), (PENTACHLOROPHENOL:Prophylaxis:0.14), (PENTETRAZO L:Prophylaxis:0.68), (PENTOBARBITAL SODIUM:Prophylaxis:0.08), (PENTOXYVERINE CITRATE:Prophylaxis:1.2), (PERGOLIDE:Prophylaxis:0.2), (PHENACETIN:Prophylaxis:0.14), (PHENAGLYCODOL:Prophylaxis:0.23), (PHENDIMETRAZINE:Prophylaxis:0.32), (PHENET HICILLIN POTASSIUM:Prophylaxis:0.57), (PHENOXYBENZAMINE:Prophylaxis:0.81), (PHENYLBUT AZONE:Prophylaxis:1.03), (PHENYLEPHRINE HYDROCHLORIDEPROMETHAZINE HYDROCHLORIDE:Prophylaxis:0.57), (PHENYLEPHRINE HYDROCHLORIDEWITCH HAZEL:Prophylaxis:0.99), (PHENYLPROPANOLAMINE:Prophylaxis:0.12), (PHOSPHAZIDE: Prophylaxis:0.4), (PHOSPHORIC ACID:Prophylaxis:0.39), (PICOLAMINE SALICYLATE:Prophylaxis:0.84), (PILSICAINIDE:Prophylaxis:1.36), (PIPOBROMAN:Prophyl axis:0.78), (PIPOTIAZINE:Prophylaxis:0.51), (PIPOTIAZINE PALMITATE:Prophylaxis:0.12), (PIRBUTEROL ACETATE:Prophylaxis:1.13), (PIRETANIDE:Prophylaxis:1.4), (PIRMENOL HYDROCHLORIDE:Prophylaxis:0.77), (PIXANTRONE:Prophylaxis:0.99), (PIZOTYLINE MALEATE:Prophylaxis:0.38), (PLATELET CONCENTRATE HUMAN:Prophylaxis:0.11), (PLATINUM:Prophylaxis:0.7), (PODOFILOX:Prophylaxis:0.9), (PO LYLACTIDE, L-:Prophylaxis:0.14), (POLYMYXIN B SULFATETRIMETHOPRIM:Prophylaxis:0.07), (POLYURETHANE:Prophylaxis:0.33), (POM EGRANATE:Prophylaxis:0.57), (POTASSIUM BICARBONATE\POTASSIUM CITRATE:Prophylaxis:0.59), (POTASSIUM BITARTRATE:Prophylaxis:0.45), (POTASSIUM BROMIDE:Prophylaxis:0.94), (POTASSIUM NITRATE\SODIUM FLUORIDE:Prophylaxis:0.82), (POTASSIUM PERMANGANATE:Prophylaxis:0.48), (POTASSIUM SULFATE:Prophylaxis:0.56), (PRACINOSTAT:Prophylaxis:0.94), (PRAMOXINE HYDROCHLORIDE:Prophylaxis:1.38), (PRAZIQUANTEL:Prophylaxis:0.13), (PREDNISOLO NE SODIUM METAZOATE:Prophylaxis:1.47), (PROCAINE HYDROCHLORIDE:Prophylaxis:0.95), (PROCYCLIDINE HYDROCHLORIDE:Prophylaxis:0.65), (PROGUANIL:Prophylaxis:0.23), (PROPARACAINE:P rophylaxis:0.23), (PROPOLIS WAX:Prophylaxis:0.74), (PROPOXYPHENE HYDROCHLORIDE:Prophylaxis:0.47), (PROPOXYPHENE NAPSYLATE:Prophylaxis:0.08), (PROPYPHENAZONE:Prophylaxis:0.25), (PROSTATE:Proph ylaxis:0.43), (PROTRIPTYLINE:Prophylaxis:0.21), (PROTRIPTYLINE HYDROCHLORIDE:Prophylaxis:1.15), (PRUCALOPRIDE:Prophylaxis:1.18), (PYRILAMINE: Prophylaxis:0.23), (QUINACRINE:Prophylaxis:1.96), (QUINAGOLIDE HYDROCHLORIDE:Prophylaxis:0.6), (QUINIDINE GLUCONATE:Prophylaxis:0.62), (QUINIDINE SULFATE:Prophylaxis:0.47), (RACEPINEPHRINE HYDROCHLORIDE:Prophylaxis:0.3), (RADIUM:Prophylaxis:0.48), (RALTITREXED:Prophyla xis:1.26), (RANOLAZINE HYDROCHLORIDE:Prophylaxis:0.66), (RASPBERRY KETONE:Prophylaxis:0.36), (RED CLOVER:Prophylaxis:0.3), (REFAMETINIB:Prophylaxis:0.74), (REGADENOSON:Prophylaxis: 0.47), (RESERPINE:Prophylaxis:0.77), (RESLIZUMAB:Prophylaxis:0.58), (RETEPLASE:Proph ylaxis:0.77), (RIBOFLAVIN 5′-PHOSPHATE SODIUM:Prophylaxis:0.08), (RIFAMYCIN SODIUM:Prophylaxis:0.39), (RIGOSERTIB:Prophylaxis:0.83), (RILONACEPT:Prophylaxis:0.77), (RILOTUMUMAB:Prophylaxis:1.26), (RIMANTADINE HYDROCHLORIDE:Prophylaxis:0.73), (RIMEXOLONE:Prophylaxis:0.43), (ROMOSOZUMA B:Prophylaxis:0.37), (ROSIGLITAZONE:Prophylaxis:0.76), (RUPATADINE FUMARATE:Prophylaxis:1.65), (RUSCUS ACULEATUS ROOT:Prophylaxis:0.86), (RUXOLITINIB PHOSPHATE:Prophylaxis:0.58), (SACROSIDASE:Prophylaxis:0.2), (SAFINAMIDE:Prophylaxi s:0.76), (SALICYLIC ACIDTRIAMCINOLONE ACETONIDE:Prophylaxis:0.49), (SAMARIUM SM-153 LEXIDRONAM PENTASODIUM:Prophylaxis:0.75), (SAVOLITINIB:Prophylaxis:0.9), (SECOBARBITAL:Prop hylaxis:0.21), (SECOBARBITAL SODIUM:Prophylaxis:0.11), (SELINEXOR:Prophylaxis:0.72), (SENNOSIDE ASENNOSIDE BSENNOSIDES:Prophylaxis:1.02), (SENNOSIDE B:Prophylaxis:1.8), (SERENOA REPENS WHOLE:Prophylaxis:0.74), (SERMORELIN ACETATE:Prophylaxis:0.08), (SEROTONIN:Prophylaxis:0.63), (SERTACONAZOLE NITRATE:Prophylaxis:0.83), (SERTINDOLE:Prophylaxis:0.38), (SESAME OIL:Prophylaxis:0.89), (SHARK CARTILAGESHARK, UNSPECIFIED:Prophylaxis:0.68), (SIBUTRAMINE HYDROCHLORIDE:Prophylaxis:1.37), (SILICON:Prophylaxis:0.65), (SILTUXIMAB:Prophyla xis:1.05), (SIMEPREVIR SODIUM:Prophylaxis:0.95), (SIMVASTATINVALSARTAN:Prophylaxis:0.5), (SINCALIDE:P rophylaxis:0.1), (SIRUKUMAB:Prophylaxis:0.58), (SOBUZOXANE:Prophylaxis:1.19), (SODIU M BENZOATE\SODIUM PHENYLACETATE:Prophylaxis:0.43), (SODIUM FLUORIDEVITAMINS:Prophylaxis:0.33), (SODIUM HYDROXIDE:Prophylaxis:0.37), (SODIUM SALICYLATE:Prophylaxis:0.21), (SODIUM SULFIDE:Prophylaxis:0.08), (SOFOSBUVIRVELPATASVIRVOXILAPREVIR:Prophylaxis:00.3), (SOMATREM:Prophylaxis:0.51), (SORAFENIB TOSYLATE:Prophylaxis:0.98), (SOY ISOFLAVONES:Prophylaxis:0.55), (STARCH, CORN:Prophylaxis:0.21), (STIRIPENTOL:Prophylaxis:0.4), (STREPTOKINASE:Prophylaxis:1), (STREPTOZOCIN:Prophylaxis:0.91), (STRONTIUM:Prophylaxis:0.62), (SULFUR:Prophylaxis: 0.5), (SULPROSTONE:Prophylaxis:0.72), (TANESPIMYCIN:Prophylaxis:0.58), (TANEZUMAB: Prophylaxis:0.85), (TAR:Prophylaxis:0.57), (TASELISIB:Prophylaxis:0.83), (TEA TREE OIL:Prophylaxis:0.4), (TECHNETIUM TC-99M EXAMETAZIME:Prophylaxis:0.07), (TECHNETIUM TC-99M TETROFOSMINTETROFOSMIN:Prophylaxis:0.16), (TECHNETIUM TC-99M TILMANOCEPT:Prophylaxis:0.31), (TEDIZOLID:Prophylaxis:0.93), (TEDIZOLID PHOSPHATE:Prophylaxis:0.49), (TEGASEROD MALEATE:Prophylaxis:1.3), (TELOTRISTAT ETHYL:Prophylaxis:1.41), (TEMOCAPRIL:Prophylaxis:0.96), (TEMOCILLIN:Prophylaxis:0.55), (TENOFOVIR ALAFENAMIDE:Prophylaxis:0.26), (TENOXICAM:Prophylaxis:1.16), (TETRAHYDROZOLI NE HYDROCHLORIDE\ZINC SULFATE:Prophylaxis:0.37), (THIABENDAZOLE:Prophylaxis:0.93), (THIAMYLAL:Prophyla xis:1.17), (THIORIDAZINE:Prophylaxis:0.27), (THIOTHIXENE HYDROCHLORIDE:Prophylaxis:0.74), (THROMBIN HUMAN:Prophylaxis:0.77), (THYROGLOBULIN:Prophylaxis:0.21), (TIAGABINE:Prophylaxis: 0.13), (TIAGABINE HYDROCHLORIDE:Prophylaxis:0.86), (TICARCILLIN:Prophylaxis:0.11), (TOLAZAMIDE:Pr ophylaxis:0.06), (TOLCAPONE:Prophylaxis:0.92), (TOLMETIN:Prophylaxis:0.84), (TOLMETIN SODIUM:Prophylaxis:0.47), (TOREMIFENE:Prophylaxis:0.93), (TOSITUMOMABTOSITUM OMAB I-131:Prophylaxis:0.46), (TRANYLCYPROMINE SULFATE:Prophylaxis:0.47), (TRAPIDIL:Prophylaxis:0.96), (TRELAGLIPTIN SUCCINATE:Prophylaxis:0.86), (TRENBOLONE:Prophylaxis:0.03), (TRIBASIC CALCIUM PHOSPHATE:Prophylaxis:0.33), (TRICAPRYLIN:Prophylaxis:0.5), (TRIENTINE:Prophylaxis:00.7), (TRIFLUOPERAZINETRIFLUOPERAZINE HYDROCHLORIDE:Prophylaxis:0.74), (TRIMETHOBENZAMIDE:Prophylaxis:1.05), (TROG LITAZONE:Prophylaxis:0.6), (TROLAMINE:Prophylaxis:0.82), (TROLAMINE SALICYLATE:Prophylaxis:0.51), (TROPINE BENZYLATE:Prophylaxis:0.14), (TRYPSIN:Prophylaxis:1.1), (TRYPTOPHAN:Prophylaxis:0.96), (ULIPRISTAL:Prophylaxis:0.96), (UNDECYLENIC ACID:Prophylaxis:0.53), (UNOPROSTONE ISOPROPYL:Prophylaxis:0.09), (UPROSERTIB:Prophylaxis:0.96), (URACIL:Prophylaxis:1.08), (URELUMAB:Prophylaxis:0.99), (URIDINE TRIACETATE:Prophylaxis:0.51), (UROFOLLITROPIN:Prophylaxis:0.02), (VALERIAN EXTRACT:Prophylaxis:0.36), (VANIPREVIR:Prophylaxis:0.69), (VARENICLINE:Prophylaxis: 0.27), (VELPATASVIR:Prophylaxis:0.75), (VERUPREVIR:Prophylaxis:0.67), (VINTAFOLIDE: Prophylaxis:0.85), (VISCUM ALBUM FRUITING TOP:Prophylaxis:0.56), (VITAMIN A ACETATE:Prophylaxis:0.05), (VITAMIN A PALMITATE:Prophylaxis:0.94), (WHEAT GERM OIL:Prophylaxis:0.6), (WHEY:Prophylaxis:0.75), (XYLITOL:Prophylaxis:0.63), (ZICONOTIDE: Prophylaxis:0.28), (ZILEUTON:Prophylaxis:1.5), (ZINC CITRATE:Prophylaxis:1.14), (ZINC PICOLINATE:Prophylaxis:0.28), (ZIPRASIDONE HYDROCHLORIDE:Prophylaxis:0.87), (ZOLEDRONATE DISODIUM:Prophylaxis:1.36), (ZUCLOPENTHIXOL:Prophylaxis:1.04), (ZUCLOPENTHIXOL DECANOATE:Prophylaxis:0.75), (ZUCLOPENTHIXOL HYDROCHLORIDE:Prophylaxis:0.18), (ANASTROZOLE:Prophylaxis against dehydration:0), (BUDESONIDE:Prophylaxis against dehydration:0.29), (CALCIUM CARBONATECHOLECALCIFEROL:Prophylaxis against dehydration:0.26), (CILASTATIN SODIUMIMIPENEM:Prophylaxis against dehydration:0.16), (CLINDAMYCINCLINDAMYCIN PHOSPHATE:Prophylaxis against dehydration:0.51), (CLOPIDOGREL BISULFATE:Prophylaxis against dehydration:0.65), (DIGOXIN:Prophylaxis against dehydration:0.46), (DILTIAZEM HYDROCHLORIDE:Prophylaxis against dehydration:0.14), (FERROUS SULFATEFERROUS SULFATE, DRIED:Prophylaxis against dehydration:0.19), (NILOTINIB:Prophylaxis against dehydration:0.24), (SACUBITRILVALSARTAN:Prophylaxis against dehydration:0.01), (VINCRISTINE SULFATE:Prophylaxis against dehydration:0.45), (ADO-TRASTUZUMAB EMTANSINE:Prophylaxis against diarrhoea:0.29), (AMIODARONE HYDROCHLORIDE:Prophylaxis against diarrhoea:0.17), (AMITRIPTYLINE:Prophylaxis against diarrhoea:0.22), (ANASTROZOLE:Prophylaxis against diarrhoea:0.85), (BORTEZOMIB:Prophylaxis against diarrhoea:0.4), (BUMETANIDE:Prophylaxis against diarrhoea:0.58), (BUPROPION HYDROCHLORIDE:Prophylaxis against diarrhoea:0.47), (CALCIUMVITAMIN D:Prophylaxis against diarrhoea:0.02), (CANDESARTAN:Prophylaxis against diarrhoea:0.69), (CANDESARTAN CILEXETILHYDROCHLOROTHIAZIDE:Prophylaxis against diarrhoea:0.08), (CEFOTAXIME SODIUM:Prophylaxis against diarrhoea:0.08), (CEPHALEXIN:Prophylaxis against diarrhoea:1.01), (CERTOLIZUMAB PEGOL:Prophylaxis against diarrhoea:0.43), (CILASTATIN SODIUMIMIPENEM:Prophylaxis against diarrhoea:0.37), (CIMETIDINE:Prophylaxis against diarrhoea:0.02), (CLOTRIMAZOLE:Prophylaxis against diarrhoea:0.38), (CLOZAPINE:Prophylaxis against diarrhoea:0.21), (CRIZOTINIB:Prophylaxis against diarrhoea:0.18), (DALTEPARIN SODIUM:Prophylaxis against diarrhoea:0.28), (DASATINIB:Prophylaxis against diarrhoea:0.63), (DICLOFENAC SODIUM:Prophylaxis against diarrhoea:0.2), (DIVALPROEX SODIUM:Prophylaxis against diarrhoea:0.16), (DOXAZOSIN MESYLATE:Prophylaxis against diarrhoea:0.03), (ENALAPRIL:Prophylaxis against diarrhoea:1.55), (EPINEPHRINE:Prophylaxis against diarrhoea:0.59), (EVEROLIMUS:Prophylaxis against diarrhoea:1.15), (EZETIMIBE:Prophylaxis against diarrhoea:0.03), (FERROUS SULFATEFERROUS SULFATE, DRIED:Prophylaxis against diarrhoea:1.06), (HYDROCHLOROTHIAZIDELISINOPRIL:Prophylaxis against diarrhoea:0.16), (ILOPROST:Prophylaxis against diarrhoea:0.26), (LEDIPASVIRSOFOSBUVIR:Prophylaxis against diarrhoea:0.3), (LINAGLIPTIN:Prophylaxis against diarrhoea:0.41), (LITHIUM CARBONATE:Prophylaxis against diarrhoea:0.08), (LOVASTATIN:Prophylaxis against diarrhoea:0.47), (MESNA:Prophylaxis against diarrhoea:0.21), (METHADONE HYDROCHLORIDE:Prophylaxis against diarrhoea:0.08), (NEBIVOLOL:Prophylaxis against diarrhoea:0.29), (OXCARBAZEPINE:Prophylaxis against diarrhoea:0.45), (PANITUMUMAB:Prophylaxis against diarrhoea:0.24), (PANTOPRAZOLE:Prophylaxis against diarrhoea:1.12), (PANTOPRAZOLE MAGNESIUM:Prophylaxis against diarrhoea:0.31), (ROFLUMILAST:Prophylaxis against diarrhoea:0.07), (SACUBITRILVALSARTAN:Prophylaxis against diarrhoea:0.08), (SELEXIPAG:Prophylaxis against diarrhoea:0.17), (THALIDOMIDE:Prophylaxis against diarrhoea:0.82), (TOPIRAMATE:Prophylaxis against diarrhoea:0.78), (VANCOMYCIN HYDROCHLORIDE:Prophylaxis against diarrhoea:0.4), (.ALPHA.1-PROTEINASE INHIBITOR HUMAN:Prophylaxis against gastrointestinal ulcer:1.05), (ACETAMINOPHENAPRONALIDECAFFEINEPROPYPHENAZONE:Prophyla xis against gastrointestinal ulcer:0.91), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Prophylaxis against gastrointestinal ulcer:0.09), (ACETAMINOPHENOXYCODONE HYDROCHLORIDEOXYCODONE TEREPHTHALATE:Prophylaxis against gastrointestinal ulcer:0.51), (ACETAMINOPHENOXYCODONEOXYCODONE HYDROCHLORIDE:Prophylaxis against gastrointestinal ulcer:1.44), (ACETAMINOPHENPSEUDOEPHEDRINE HYDROCHLORIDE:Prophylaxis against gastrointestinal ulcer:0.21), (ACLARUBICIN HYDROCHLORIDE:Prophylaxis against gastrointestinal ulcer:0.63), (ACOTIAMIDE HYDROCHLORIDE:Prophylaxis against gastrointestinal ulcer:0.04), (ADENINE:Prophylaxis against gastrointestinal ulcer:1.46), (ADUCANUMAB:Prophylaxis against gastrointestinal ulcer:0.11), (AGALSIDASE BETA:Prophylaxis against gastrointestinal ulcer:0.94), (ALDESLEUKIN:Prophylaxis against gastrointestinal ulcer:0.69), (ALGLUCOSIDASE ALFA:Prophylaxis against gastrointestinal ulcer:0.62), (ALUMINUM HYDROXIDEMAGNESIUM OXIDEOXETHAZAINE:Prophylaxis against gastrointestinal ulcer:0.51), (AMISULPRIDE:Prophylaxis against gastrointestinal ulcer:1.52), (AMLODIPINE BESYLATEATORVASTATIN CALCIUM:Prophylaxis against gastrointestinal ulcer:1.1), (AMPICILLIN SODIUM:Prophylaxis against gastrointestinal ulcer:0.36), (ANAGLIPTIN:Prophylaxis against gastrointestinal ulcer:1.28), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Prophylaxis against gastrointestinal ulcer:0.07), (ASPARAGINASE:Prophylaxis against gastrointestinal ulcer:0.61), (ASPIRINDIHYDROXYALUMINUM AMINOACETATEMAGNESIUM CARBONATE:Prophylaxis against gastrointestinal ulcer:0.1), (ASPIRINLANSOPRAZOLE:Prophylaxis against gastrointestinal ulcer:1.29), (ATRACURIUM:Prophylaxis against gastrointestinal ulcer:0.64), (ATRACURIUM BESYLATE:Prophylaxis against gastrointestinal ulcer:0.45), (ATROPINEDIPHENOXYLATE:Prophylaxis against gastrointestinal ulcer:0.24), (AVOCADO OILSOYBEAN OIL:Prophylaxis against gastrointestinal ulcer:0.18), (AZITHROMYCIN OR METHOTREXATE SODIUM:Prophylaxis against gastrointestinal ulcer:0.71), (BELATACEPT:Prophylaxis against gastrointestinal ulcer:0.68), (BENIDIPINE:Prophylaxis against gastrointestinal ulcer:1.18), (BENPROPERINE PHOSPHATE:Prophylaxis against gastrointestinal ulcer:0.52), (BENZTROPINE:Prophylaxis against gastrointestinal ulcer:0.3), (BENZTROPINE MESYLATE:Prophylaxis against gastrointestinal ulcer:0.04), (BETAMETHASONE DIPROPIONATECALCIPOTRIENE:Prophylaxis against gastrointestinal ulcer:0.4), (BETAMETHASONE DIPROPIONATECALCIPOTRIENECALCIPOTRIENE HYDRATE:Prophylaxis against gastrointestinal ulcer:0.49), (BETAMETHASONE DIPROPIONATECLOTRIMAZOLE:Prophylaxis against gastrointestinal ulcer:0.69), (BETAMETHASONE SODIUM PHOSPHATENEOMYCIN SULFATE:Prophylaxis against gastrointestinal ulcer:1.1), (BETAMETHASONECALCIPOTRIENE:Prophylaxis against gastrointestinal ulcer:0.72), (BIAPENEM:Prophylaxis against gastrointestinal ulcer:0.52), (BICARBONATE ION:Prophylaxis against gastrointestinal ulcer:0.87), (BICARBONATE IONPOLYETHYLENE GLYCOLSPOTASSIUM CATION\SODIUM CHLORIDE\SODIUM SULFATE ANHYDROUS:Prophylaxis against gastrointestinal ulcer:0.43), (LONGUM SUBSP. INFANTIS:Prophylaxis against gastrointestinal ulcer:0.35), (BIMATOPROSTTIMOLOL:Prophylaxis against gastrointestinal ulcer:0.62), (BIPERIDEN:Prophylaxis against gastrointestinal ulcer:0.1), (BIXALOMER:Prophylaxis against gastrointestinal ulcer:0.38), (BLESELUMAB:Prophylaxis against gastrointestinal ulcer:0.33), (BLINATUMOMAB:Prophylaxis against gastrointestinal ulcer:1.31), (BROMPERIDOL:Prophylaxis against gastrointestinal ulcer:0.01), (BUFORMIN:Prophylaxis against gastrointestinal ulcer:0.48), (BUPIVACAINE HYDROCHLORIDE:Prophylaxis against gastrointestinal ulcer:0.16), (BUPRENORPHINE HYDROCHLORIDENALOXONE HYDROCHLORIDE:Prophylaxis against gastrointestinal ulcer:0.45), (CABOZANTINIB:Prophylaxis against gastrointestinal ulcer:0.04), (CALCITONIN:Prophylaxis against gastrointestinal ulcer:0.92), (CALCITONIN SALMON:Prophylaxis against gastrointestinal ulcer:1.36), (CALCIUM CARBONATEERGOCALCIFEROLRETINOL:Prophylaxis against gastrointestinal ulcer:0.42), (CALCIUM CARBONATEVITAMIN D:Prophylaxis against gastrointestinal ulcer:1.04), (CALCIUM\MAGNESIUM:Prophylaxis against gastrointestinal ulcer:0.5), (CALCIUM\MAGNESIUM\ZINC:Prophylaxis against gastrointestinal ulcer:0.18), (CAMPHORCAPSICUMMETHYL SALICYLATE:Prophylaxis against gastrointestinal ulcer:0.15), (CAMPHORMENTHOLMETHYL SALICYLATE:Prophylaxis against gastrointestinal ulcer:1.15), (CANAGLIFLOZINMETFORMIN HYDROCHLORIDE:Prophylaxis against gastrointestinal ulcer:1.08), (CARMUSTINE:Prophylaxis against gastrointestinal ulcer:0.13), (CARPRONIUM CHLORIDE:Prophylaxis against gastrointestinal ulcer:0.17), (CARTEOLOL:Prophylaxis against gastrointestinal ulcer:0.01), (CARVEDILOL PHOSPHATE:Prophylaxis against gastrointestinal ulcer:2.08), (CEFOTAXIMECEFOTAXIME SODIUM:Prophylaxis against gastrointestinal ulcer:0.84), (CEFTERAM PIVOXIL:Prophylaxis against gastrointestinal ulcer:0.32), (CELIPROLOL:Prophylaxis against gastrointestinal ulcer:0.52), (CELLULASEDIASTASELIPASE:Prophylaxis against gastrointestinal ulcer:1.36), (CEPHARANTHINE:Prophylaxis against gastrointestinal ulcer:1.29), (CETRAXATE HYDROCHLORIDE:Prophylaxis against gastrointestinal ulcer:0.22), (CHLORPHENIRAMINE MALEATDIHYDROCODEINE PHOSPHATEMETHYLEPHEDRINE HYDROCHLORIDE, DL-:Prophylaxis against gastrointestinal ulcer:0.71), (CHLORPHENIRAMINEHYDROCODONE:Prophylaxis against gastrointestinal ulcer:0.28), (CHLORPROMAZINEPROMETHAZINE:Prophylaxis against gastrointestinal ulcer:0.18), (CHOLINE BITARTRATECYSTEINEINOSITOLMAMMAL LIVER:Prophylaxis against gastrointestinal ulcer:0.55), (CIFENLINE SUCCINATE:Prophylaxis against gastrointestinal ulcer:0.29), (CLADRIBINE:Prophylaxis against gastrointestinal ulcer:0.12), (CLENBUTEROL HYDROCHLORIDE:Prophylaxis against gastrointestinal ulcer:1.67), (CLOFARABINE:Prophylaxis against gastrointestinal ulcer:0.66), (CLOMIPRAMINE HYDROCHLORIDE:Prophylaxis against gastrointestinal ulcer:0.82), (CLOPERASTINE:Prophylaxis against gastrointestinal ulcer:0.96), (CLOXACILLIN SODIUM:Prophylaxis against gastrointestinal ulcer:0.21), (COD LIVER OIL:Prophylaxis against gastrointestinal ulcer:0.23), (CODEINE PHOSPHATEGUAIFENESIN:Prophylaxis against gastrointestinal ulcer:0.31), (COLESTILAN CHLORIDE:Prophylaxis against gastrointestinal ulcer:0.63), (CORTICOTROPIN:Prophylaxis against gastrointestinal ulcer:0.47), (CYANAMIDE:Prophylaxis against gastrointestinal ulcer:0.21), (CYPROHEPTADINE:Prophylaxis against gastrointestinal ulcer:0.84), (CYPROHEPTADINE HYDROCHLORIDE:Prophylaxis against gastrointestinal ulcer:0.31), (CYPROTERONE ACETATE:Prophylaxis against gastrointestinal ulcer:0.46), (CYSTEINEGLYCINEGLYCYRRHIZIN:Prophylaxis against gastrointestinal ulcer:0.01), (CYTARABINE OCFOSFATE:Prophylaxis against gastrointestinal ulcer:0.18), (DABRAFENIB MESYLATE:Prophylaxis against gastrointestinal ulcer:0.23), (DACLIZUMAB:Prophylaxis against gastrointestinal ulcer:1.48), (DAPAGLIFLOZIN PROPANEDIOL:Prophylaxis against gastrointestinal ulcer:0.6), (DEFIBROTIDE (BOVINE):Prophylaxis against gastrointestinal ulcer:0.04), (DESFLURANE:Prophylaxis against gastrointestinal ulcer:0.9), (DESOXIMETASONE:Prophylaxis against gastrointestinal ulcer:0.21), (DEXTROAMPHETAMINE SULFATE:Prophylaxis against gastrointestinal ulcer:0.17), (DIMETHICONESILICON DIOXIDESIMETHICONE:Prophylaxis against gastrointestinal ulcer:0.38), (DIPHENHYDRAMINE HYDROCHLORIDELIDOCAINE HYDROCHLORIDENYSTATIN:Prophylaxis against gastrointestinal ulcer:0.11), (DOCUSATE CALCIUM:Prophylaxis against gastrointestinal ulcer:1.39), (DOLASETRON MESYLATE:Prophylaxis against gastrointestinal ulcer:0.43), (DOLUTEGRAVIR:Prophylaxis against gastrointestinal ulcer:0.26), (DOXEPIN HYDROCHLORIDE:Prophylaxis against gastrointestinal ulcer:0.55), (DRONABINOL:Prophylaxis against gastrointestinal ulcer:1.66), (ECONAZOLE:Prophylaxis against gastrointestinal ulcer:1.81), (EFAVIRENZEMTRICITABINETENOFOVIR DISOPROXIL FUMARATE:Prophylaxis against gastrointestinal ulcer:0.63), (ELETRIPTAN HYDROBROMIDE:Prophylaxis against gastrointestinal ulcer:1.31), (ELTROMBOPAG OLAMINE:Prophylaxis against gastrointestinal ulcer:0.11), (EMPAGLIFLOZINLINAGLIPTIN:Prophylaxis against gastrointestinal ulcer:0.6), (EPHEDRINE HYDROCHLORIDE:Prophylaxis against gastrointestinal ulcer:0.4), (ERYTHROMYCINSULFISOXAZOLE:Prophylaxis against gastrointestinal ulcer:0.41), (ESMOLOL HYDROCHLORIDE:Prophylaxis against gastrointestinal ulcer:1.3), (ESTROGENS:Prophylaxis against gastrointestinal ulcer:0.1), (ESTRONESCROFA OVARY:Prophylaxis against gastrointestinal ulcer:0.25), (ETHACRYNIC ACID:Prophylaxis against gastrointestinal ulcer:0.32), (ETOPOSIDE PHOSPHATE:Prophylaxis against gastrointestinal ulcer:0.23), (ETRETINATE:Prophylaxis against gastrointestinal ulcer:2.18), (FERRIC PYROPHOSPHATE:Prophylaxis against gastrointestinal ulcer:0.08), (FERROUS SULFATE ANHYDROUS:Prophylaxis against gastrointestinal ulcer:0.23), (FIBRINOGEN HUMAN:Prophylaxis against gastrointestinal ulcer:0.44), (FLECAINIDE ACETATE:Prophylaxis against gastrointestinal ulcer:0.29), (FLOMOXEF:Prophylaxis against gastrointestinal ulcer:0.29), (FLOPROPIONE:Prophylaxis against gastrointestinal ulcer:0.18), (FLUDROCORTISONE ACETATE:Prophylaxis against gastrointestinal ulcer:1.43), (FLUOCINOLONE ACETONIDE:Prophylaxis against gastrointestinal ulcer:0.81), (FLUTICASONE:Prophylaxis against gastrointestinal ulcer:1.65), (FLUTICASONE PROPIONATEFORMOTEROL FUMARATE:Prophylaxis against gastrointestinal ulcer:0.53), (FORMOTEROL FUMARATE\MOMETASONE FUROATE:Prophylaxis against gastrointestinal ulcer:0.81), (FOSFOMYCIN SODIUM:Prophylaxis against gastrointestinal ulcer:0), (FOSINOPRIL:Prophylaxis against gastrointestinal ulcer:0.24), (FOSPHENYTOINFOSPHENYTOIN SODIUM:Prophylaxis against gastrointestinal ulcer:1.18), (FULVESTRANT:Prophylaxis against gastrointestinal ulcer:0.84), (FURSULTIAMINE:Prophylaxis against gastrointestinal ulcer:1.49), (GARENOXACIN:Prophylaxis against gastrointestinal ulcer:0.58), (GLYCOPYRROLATEINDACATEROL MALEATE:Prophylaxis against gastrointestinal ulcer:0.55), (GLYCYRRHIZIN:Prophylaxis against gastrointestinal ulcer:0.29), (GUANFACINE HYDROCHLORIDE:Prophylaxis against gastrointestinal ulcer:0.06), (HERBALSSAW PALMETTO:Prophylaxis against gastrointestinal ulcer:0.86), (HERBALSTURMERIC:Prophylaxis against gastrointestinal ulcer:0.6), (HUMAN BLOOD CELLS:Prophylaxis against gastrointestinal ulcer:0.13), (HYDROCORTISONE ACETATE:Prophylaxis against gastrointestinal ulcer:1.06), (HYDROCORTISONE HEMISUCCINATE:Prophylaxis against gastrointestinal ulcer:0.07), (HYDROCORTISONE PROBUTATE:Prophylaxis against gastrointestinal ulcer:0.32), (IBRITUMOMAB TIUXETAN:Prophylaxis against gastrointestinal ulcer:1.28), (IBUDILAST:Prophylaxis against gastrointestinal ulcer:0.64), (IDARUCIZUMAB:Prophylaxis against gastrointestinal ulcer:1.08), (IFENPRODIL TARTRATE:Prophylaxis against gastrointestinal ulcer:0.55), (ILOPROST:Prophylaxis against gastrointestinal ulcer:1.44), (IMIGLUCERASE:Prophylaxis against gastrointestinal ulcer:0.66), (IMIPRAMINE HYDROCHLORIDE:Prophylaxis against gastrointestinal ulcer:0.86), (INSULIN ASPARTINSULIN DEGLUDEC:Prophylaxis against gastrointestinal ulcer:0.54), (INSULIN BEEF:Prophylaxis against gastrointestinal ulcer:0.94), (INTERFERON:Prophylaxis against gastrointestinal ulcer:1.16), (INTERFERON ALFA-2A:Prophylaxis against gastrointestinal ulcer:1.72), (INTERFERON ALFA-2B:Prophylaxis against gastrointestinal ulcer:0.92), (INTERFERON ALFA-N1:Prophylaxis against gastrointestinal ulcer:0.42), (INTERFERON GAMMA-1B:Prophylaxis against gastrointestinal ulcer:0.13), (IPRAGLIFLOZIN L-PROLINE:Prophylaxis against gastrointestinal ulcer:0.78), (ISONIAZIDPYRAZINAMIDERIFAMPIN:Prophylaxis against gastrointestinal ulcer:0.4), (KALLIDINOGENASE:Prophylaxis against gastrointestinal ulcer:0.94), (LACTASE:Prophylaxis against gastrointestinal ulcer:0.35), (RHAMNOSUS:Prophylaxis against gastrointestinal ulcer:0.71), (LANREOTIDE:Prophylaxis against gastrointestinal ulcer:0.19), (LANREOTIDE ACETATE:Prophylaxis against gastrointestinal ulcer:0.2), (LENVATINIB:Prophylaxis against gastrointestinal ulcer:0.77), (LEVALBUTEROL:Prophylaxis against gastrointestinal ulcer:0.45), (LEVAMLODIPINE BESYLATE:Prophylaxis against gastrointestinal ulcer:0.24), (LEVOMEFOLATE CALCIUM\METHYLCOBALAMINPYRIDOXAL PHOSPHATE ANHYDROUS:Prophylaxis against gastrointestinal ulcer:0.07), (LEVOMEPROMAZINE:Prophylaxis against gastrointestinal ulcer:0.47), (LEVOSIMENDAN:Prophylaxis against gastrointestinal ulcer:0.56), (LINSIDOMINE:Prophylaxis against gastrointestinal ulcer:0.66), (LIOTHYRONINE:Prophylaxis against gastrointestinal ulcer:0.04), (LISDEXAMFETAMINE DIMESYLATE:Prophylaxis against gastrointestinal ulcer:0.35), (LOMUSTINE:Prophylaxis against gastrointestinal ulcer:0.15), (MAGNESIUM ASPARTATE\POTASSIUM ASPARTATE:Prophylaxis against gastrointestinal ulcer:0.65), (MAGNESIUM ISOGLYCYRRHIZINATE:Prophylaxis against gastrointestinal ulcer:0.09), (MAGNESIUM PIDOLATE:Prophylaxis against gastrointestinal ulcer:0.23), (MEPOLIZUMAB:Prophylaxis against gastrointestinal ulcer:0.72), (METAXALONE:Prophylaxis against gastrointestinal ulcer:1.27), (METFORMIN HYDROCHLORIDESAXAGLIPTIN:Prophylaxis against gastrointestinal ulcer:0.19), (METFORMIN HYDROCHLORIDESAXAGLIPTIN HYDROCHLORIDE:Prophylaxis against gastrointestinal ulcer:0.14), (METHENAMINE HIPPURATE:Prophylaxis against gastrointestinal ulcer:0.95), (METRELEPTIN:Prophylaxis against gastrointestinal ulcer:0.06), (METYRAPONE:Prophylaxis against gastrointestinal ulcer:0.1), (MIGLUSTAT:Prophylaxis against gastrointestinal ulcer:0.38), (MIRIPLATIN:Prophylaxis against gastrointestinal ulcer:0.48), (MOEXIPRIL HYDROCHLORIDE:Prophylaxis against gastrointestinal ulcer:0.04), (MOSAPRIDE CITRATE DIHYDRATE:Prophylaxis against gastrointestinal ulcer:0.86), (NADOLOL:Prophylaxis against gastrointestinal ulcer:1.05), (NALDEMEDINE:Prophylaxis against gastrointestinal ulcer:0.15), (NALFURAFINE HYDROCHLORIDE:Prophylaxis against gastrointestinal ulcer:0.25), (NILVADIPINE:Prophylaxis against gastrointestinal ulcer:0.32), (NITROFURANTOINNITROFURANTOIN MONOHYDRATE:Prophylaxis against gastrointestinal ulcer:0.45), (NORETHINDRONE:Prophylaxis against gastrointestinal ulcer:0.11), (NORTRIPTYLINE:Prophylaxis against gastrointestinal ulcer:1.21), (OCTOTIAMINE:Prophylaxis against gastrointestinal ulcer:0.33), (OPIPRAMOL:Prophylaxis against gastrointestinal ulcer:0.53), (ORGOTEIN:Prophylaxis against gastrointestinal ulcer:0.14), (ORPHENADRINE:Prophylaxis against gastrointestinal ulcer:0.33), (OXATOMIDE:Prophylaxis against gastrointestinal ulcer:0.04), (OXYBUTYNIN CHLORIDE:Prophylaxis against gastrointestinal ulcer:1.23), (OXYMETAZOLINE:Prophylaxis against gastrointestinal ulcer:0.19), (PANTHENOL:Prophylaxis against gastrointestinal ulcer:0.23), (PATIROMER:Prophylaxis against gastrointestinal ulcer:0.28), (PEGINTERFERON BETA-1A:Prophylaxis against gastrointestinal ulcer:0.69), (PENICILLIN:Prophylaxis against gastrointestinal ulcer:1.96), (PENTOSTATIN:Prophylaxis against gastrointestinal ulcer:0.93), (PENTOXYVERINE CITRATE:Prophylaxis against gastrointestinal ulcer:0.03), (PEROSPIRONE HYDROCHLORIDE:Prophylaxis against gastrointestinal ulcer:0.25), (PHENOTHIAZINE:Prophylaxis against gastrointestinal ulcer:0.45), (PHOSPHOCREATINE SODIUM:Prophylaxis against gastrointestinal ulcer:0.23), (PILOCARPINE:Prophylaxis against gastrointestinal ulcer:0.57), (PILSICAINIDE:Prophylaxis against gastrointestinal ulcer:0.19), (PIMECROLIMUS:Prophylaxis against gastrointestinal ulcer:0.01), (POLYETHYLENE GLYCOL 3350POTASSIUM CHLORIDE\SODIUM BICARBONATE\SODIUM CHLORIDE\SODIUM SULFATE ANHYDROUS:Prophylaxis against gastrointestinal ulcer:0.42), (PORFIMER SODIUM:Prophylaxis against gastrointestinal ulcer:0.83), (PRANOPROFEN:Prophylaxis against gastrointestinal ulcer:0.05), (PRAZOSIN:Prophylaxis against gastrointestinal ulcer:0.79), (PRIMIDONE:Prophylaxis against gastrointestinal ulcer:1.73), (PROBUCOL:Prophylaxis against gastrointestinal ulcer:0.97), (PROCATEROL HYDROCHLORIDE:Prophylaxis against gastrointestinal ulcer:0.83), (PROPAFENONE HYDROCHLORIDE:Prophylaxis against gastrointestinal ulcer:0.85), (PROPIVERINE:Prophylaxis against gastrointestinal ulcer:1.27), (PSYLLIUM HUSK:Prophylaxis against gastrointestinal ulcer:0.82), (PYGEUM:Prophylaxis against gastrointestinal ulcer:0.01), (PYRAZINAMIDE:Prophylaxis against gastrointestinal ulcer:0.78), (PYRIDOXAL PHOSPHATE:Prophylaxis against gastrointestinal ulcer:0.49), (QUERCUS SALICINA STEM:Prophylaxis against gastrointestinal ulcer:0.35), (QUINACRINE:Prophylaxis against gastrointestinal ulcer:0.07), (RETINOL:Prophylaxis against gastrointestinal ulcer:0.79), (ROPIVACAINE HYDROCHLORIDE:Prophylaxis against gastrointestinal ulcer:0.62), (SALMETEROL:Prophylaxis against gastrointestinal ulcer:1.17), (SELEGILINE:Prophylaxis against gastrointestinal ulcer:0.13), (SELEGILINE HYDROCHLORIDE:Prophylaxis against gastrointestinal ulcer:0.72), (SELEXIPAG:Prophylaxis against gastrointestinal ulcer:0.69), (SENNOSIDE B:Prophylaxis against gastrointestinal ulcer:0.55), (SERRAPEPTASE:Prophylaxis against gastrointestinal ulcer:0.39), (SEVELAMER:Prophylaxis against gastrointestinal ulcer:0.32), (SIPULEUCEL-T:Prophylaxis against gastrointestinal ulcer:0.15), (SODIUM FLUORIDE:Prophylaxis against gastrointestinal ulcer:0.67), (SODIUM OXYBATE:Prophylaxis against gastrointestinal ulcer:0.82), (SOMATOSTATIN:Prophylaxis against gastrointestinal ulcer:0), (SORBITOL:Prophylaxis against gastrointestinal ulcer:0.58), (SOTALOL HYDROCHLORIDE:Prophylaxis against gastrointestinal ulcer:0.87), (STREPTOMYCIN SULFATE:Prophylaxis against gastrointestinal ulcer:0.15), (SULBACTAM SODIUM:Prophylaxis against gastrointestinal ulcer:0.2), (SULFAMERAZINETRIMETHOPRIM:Prophylaxis against gastrointestinal ulcer:0.74), (SUMATRIPTAN SUCCINATE:Prophylaxis against gastrointestinal ulcer:0.16), (SUPLATAST TOSILATE:Prophylaxis against gastrointestinal ulcer:1.09), (TABALUMAB:Prophylaxis against gastrointestinal ulcer:0.09), (TAFAMIDIS MEGLUMINE:Prophylaxis against gastrointestinal ulcer:0.36), (TAFLUPROST:Prophylaxis against gastrointestinal ulcer:0.44), (TAMOXIFEN CITRATE:Prophylaxis against gastrointestinal ulcer:0.61), (TECELEUKIN:Prophylaxis against gastrointestinal ulcer:0.33), (TEDUGLUTIDE\WATER:Prophylaxis against gastrointestinal ulcer:0.11), (TESTOSTERONE CYPIONATE:Prophylaxis against gastrointestinal ulcer:0.04), (THIOCTIC ACID:Prophylaxis against gastrointestinal ulcer:0.53), (THROMBOPOIETIN:Prophylaxis against gastrointestinal ulcer:0.31), (THYROID, PORCINE:Prophylaxis against gastrointestinal ulcer:0.31), (TIARAMIDE:Prophylaxis against gastrointestinal ulcer:0.11), (TIMOLOL MALEATETRAVOPROST:Prophylaxis against gastrointestinal ulcer:0.47), (TISAGENLECLEUCEL:Prophylaxis against gastrointestinal ulcer:0.06), (TOFISOPAM:Prophylaxis against gastrointestinal ulcer:0.55), (TOPIROXOSTAT:Prophylaxis against gastrointestinal ulcer:0.42), (TRANILAST:Prophylaxis against gastrointestinal ulcer:0.11), (TRIHEXYPHENIDYL:Prophylaxis against gastrointestinal ulcer:0.62), (TRIMIPRAMINE MALEATE:Prophylaxis against gastrointestinal ulcer:1.21), (TROXIPIDE:Prophylaxis against gastrointestinal ulcer:1.02), (VIDARABINE:Prophylaxis against gastrointestinal ulcer:0.71), (VILAZODONE HYDROCHLORIDE:Prophylaxis against gastrointestinal ulcer:0.1), (VINDESINE SULFATE:Prophylaxis against gastrointestinal ulcer:0.36), (ZILEUTON:Prophylaxis against gastrointestinal ulcer:0.25), (ZINC:Prophylaxis against gastrointestinal ulcer:0.76), (ZIV-AFLIBERCEPT:Prophylaxis against gastrointestinal ulcer:0.39), (ACETAMINOPHENCODEINE PHOSPHATE:Prophylaxis against graft versus host disease:0.31), (ACETAMINOPHENHYDROCODONE BITARTRATE:Prophylaxis against graft versus host disease:0.09), (AMPHOTERICIN BDIMYRISTOYLPHOSPHATIDYLCHOLINE, DL-DIMYRISTOYLPHOSPHATIDYLGLYCEROL, DL-:Prophylaxis against graft versus host disease:0.91), (ARSENIC TRIOXIDE:Prophylaxis against graft versus host disease:0.34), (BELATACEPT:Prophylaxis against graft versus host disease:0.45), (BISACODYL OR DOCUSATE SODIUM:Prophylaxis against graft versus host disease:0), (BLESELUMAB:Prophylaxis against graft versus host disease:0.1), (BUPRENORPHINE HYDROCHLORIDENALOXONE HYDROCHLORIDE:Prophylaxis against graft versus host disease:0.02), (CALCIUM ACETATE:Prophylaxis against graft versus host disease:0.05), (CALCIUM CARBONATECHOLECALCIFEROL:Prophylaxis against graft versus host disease:0.58), (CANAKINUMAB:Prophylaxis against graft versus host disease:0.53), (CEFOZOPRAN:Prophylaxis against graft versus host disease:0.06), (CEFUROXIME:Prophylaxis against graft versus host disease:0.36), (CELECOXIB:Prophylaxis against graft versus host disease:0.26), (CERTOLIZUMAB PEGOL:Prophylaxis against graft versus host disease:0.25), (CHLORAMBUCIL:Prophylaxis against graft versus host disease:0.92), (CHOLESTYRAMINE:Prophylaxis against graft versus host disease:0.27), (CYCLOPHOSPHAMIDECYTARABINEDEXAMETHASONEDOXORUBICI NMETHOTREXATEVINCRISTINE:Prophylaxis against graft versus host disease:0.61), (DEXLANSOPRAZOLE:Prophylaxis against graft versus host disease:0.27), (DICLOFENAC:Prophylaxis against graft versus host disease:0.01), (DIVALPROEX SODIUM:Prophylaxis against graft versus host disease:0.63), (ELOTUZUMAB:Prophylaxis against graft versus host disease:0.11), (ESZOPICLONE:Prophylaxis against graft versus host disease:0.24), (ETOPOSIDE PHOSPHATE:Prophylaxis against graft versus host disease:0.58), (FERROUS SULFATE:Prophylaxis against graft versus host disease:0.5), (FERROUS SULFATEFERROUS SULFATE, DRIED:Prophylaxis against graft versus host disease:0.51), (FISH OIL:Prophylaxis against graft versus host disease:0.43), (FLUTICASONE PROPIONATE:Prophylaxis against graft versus host disease:0.58), (FOSFOMYCIN:Prophylaxis against graft versus host disease:0.55), (GUSPERIMUS TRIHYDROCHLORIDE:Prophylaxis against graft versus host disease:1.09), (IDELALISIB:Prophylaxis against graft versus host disease:0.53), (INTERFERON GAMMA-1B:Prophylaxis against graft versus host disease:0.13), (IRON:Prophylaxis against graft versus host disease:0.44), (ISAVUCONAZOLE:Prophylaxis against graft versus host disease:0.06), (LACOSAMIDE:Prophylaxis against graft versus host disease:0.02), (LIDOCAINEPRILOCAINE:Prophylaxis against graft versus host disease:0.26), (LOVASTATIN:Prophylaxis against graft versus host disease:0.09), (MELOXICAM:Prophylaxis against graft versus host disease:0.1), (METHENOLONE ENANTHATE:Prophylaxis against graft versus host disease:0.98), (METHOTREXATEMETHOTREXATE SODIUM:Prophylaxis against graft versus host disease:0.72), (MOGAMULIZUMAB:Prophylaxis against graft versus host disease:1.13), (OSELTAMIVIR:Prophylaxis against graft versus host disease:0.75), (OXCARBAZEPINE:Prophylaxis against graft versus host disease:1.07), (PLERIXAFOR:Prophylaxis against graft versus host disease:0.1), (POTASSIUM CITRATETRISODIUM CITRATE DIHYDRATE:Prophylaxis against graft versus host disease:0.03), (PRAVASTATIN:Prophylaxis against graft versus host disease:0.25), (QUETIAPINE:Prophylaxis against graft versus host disease:0.01), (RASBURICASE:Prophylaxis against graft versus host disease:0.18), (RISEDRONATE SODIUM:Prophylaxis against graft versus host disease:0.11), (SULFAMERAZINETRIMETHOPRIM:Prophylaxis against graft versus host disease:0.51), (TAZOBACTAM:Prophylaxis against graft versus host disease:0.23), (TENIPOSIDE:Prophylaxis against graft versus host disease:0.01), (THIOGUANINE ANHYDROUS:Prophylaxis against graft versus host disease:0.15), (TIOTROPIUM BROMIDE MONOHYDRATE:Prophylaxis against graft versus host disease:0.03), (TISAGENLECLEUCEL:Prophylaxis against graft versus host disease:0.6), (TOPIRAMATE:Prophylaxis against graft versus host disease:0.74), (VINDESINE SULFATE:Prophylaxis against graft versus host disease:0.81), (ABACAVIR SULFATEDOLUTEGRAVIR SODIUMLAMIVUDINE:Prophylaxis against HIV infection:0.99), (ZALCITABINE:Prophylaxis against HIV infection:0.19), (.ALPHA.1-PROTEINASE INHIBITOR HUMAN:Prophylaxis against transplant rejection:0.22), (ACETAMINOPHENOXYCODONEOXYCODONE HYDROCHLORIDE:Prophylaxis against transplant rejection:0.52), (ACETAMINOPHENPROPOXYPHENE HYDROCHLORIDE:Prophylaxis against transplant rejection:0.02), (ACETAZOLAMIDE:Prophylaxis against transplant rejection:0.98), (AGALSIDASE BETA:Prophylaxis against transplant rejection:0.47), (ALCOHOL:Prophylaxis against transplant rejection:0.22), (ALISKIREN:Prophylaxis against transplant rejection:0.04), (ALUMINUM HYDROXIDEMAGNESIUM OXIDEOXETHAZAINE:Prophylaxis against transplant rejection:0.2), (AMILORIDE HYDROCHLORIDEHYDROCHLOROTHIAZIDE:Prophylaxis against transplant rejection:0.01), (AMINO ACIDSDEXTROSEELECTROLYTES NOS:Prophylaxis against transplant rejection:0.21), (AMISULPRIDE:Prophylaxis against transplant rejection:0.38), (AMLODIPINE BESYLATEOLMESARTAN MEDOXOMIL:Prophylaxis against transplant rejection:0.06), (AMLODIPINEVALSARTAN:Prophylaxis against transplant rejection:0.14), (ANASTROZOLE:Prophylaxis against transplant rejection:0.83), (ARMODAFINIL:Prophylaxis against transplant rejection:0.7), (ATAZANAVIR:Prophylaxis against transplant rejection:0.04), (AVOBENZONEHOMOSALATEOCTISALATE:Prophylaxis against transplant rejection:0.05), (BENAZEPRILBENAZEPRIL HYDROCHLORIDE:Prophylaxis against transplant rejection:0.38), (BENZONATATE:Prophylaxis against transplant rejection:0.39), (BLESELUMAB:Prophylaxis against transplant rejection:1.47), (BUDESONIDEFORMOTEROL FUMARATE:Prophylaxis against transplant rejection:0.11), (BUTYLSCOPOLAMINE BROMIDE:Prophylaxis against transplant rejection:0.01), (CALCIUM ACETATE:Prophylaxis against transplant rejection:1.86), (CANAGLIFLOZINMETFORMIN HYDROCHLORIDE:Prophylaxis against transplant rejection:0.6), (CARFILZOMIB:Prophylaxis against transplant rejection:1.07), (CEFOPERAZONETAZOBACTAM SODIUM:Prophylaxis against transplant rejection:0.03), (CEFOTAXIMECEFOTAXIME SODIUM:Prophylaxis against transplant rejection:0.18), (CHLORAMBUCIL:Prophylaxis against transplant rejection:0.63), (CILOSTAZOL:Prophylaxis against transplant rejection:0.33), (CLOZAPINE:Prophylaxis against transplant rejection:0.96), (CODEINE:Prophylaxis against transplant rejection:0.17), (COLISTIN:Prophylaxis against transplant rejection:0.07), (DABIGATRAN:Prophylaxis against transplant rejection:0.37), (DANAZOL:Prophylaxis against transplant rejection:0.73), (DAPAGLIFLOZIN PROPANEDIOL:Prophylaxis against transplant rejection:0.17), (DASATINIB:Prophylaxis against transplant rejection:1.58), (DEFLAZACORT:Prophylaxis against transplant rejection:0.11), (DESLORATADINE:Prophylaxis against transplant rejection:0.9), (DESONIDE:Prophylaxis against transplant rejection:0.11), (DEXAMETHASONE ACETATE:Prophylaxis against transplant rejection:0.82), (DEXTROAMPHETAMINE SULFATE:Prophylaxis against transplant rejection:0.06), (DICLOFENAC:Prophylaxis against transplant rejection:1.49), (DOFETILIDE:Prophylaxis against transplant rejection:0.47), (DOXEPIN:Prophylaxis against transplant rejection:0.09), (ECONAZOLE:Prophylaxis against transplant rejection:0.87), (EDOXABAN TOSYLATE:Prophylaxis against transplant rejection:0.56), (ELETRIPTAN HYDROBROMIDE:Prophylaxis against transplant rejection:0.7), (ELOTUZUMAB:Prophylaxis against transplant rejection:1.27), (EMPAGLIFLOZINLINAGLIPTIN:Prophylaxis against transplant rejection:0.11), (ESMOLOL HYDROCHLORIDE:Prophylaxis against transplant rejection:0.48), (ESTROGENS, CONJUGATEDMEDROXYPROGESTERONE ACETATE:Prophylaxis against transplant rejection:0.21), (ETORICOXIB:Prophylaxis against transplant rejection:0.39), (EVOLOCUMAB:Prophylaxis against transplant rejection:1.15), (FENTANYL CITRATE:Prophylaxis against transplant rejection:0.03), (FERROUS SULFATEGLYCINE:Prophylaxis against transplant rejection:0.22), (FESOTERODINE FUMARATE:Prophylaxis against transplant rejection:0.61), (FLUTICASONE FUROATESALMETEROL XINAFOATE:Prophylaxis against transplant rejection:0.58), (FLUTICASONE FUROATEVILANTEROL TRIFENATATE:Prophylaxis against transplant rejection:0.74), (FORMOTEROL FUMARATE:Prophylaxis against transplant rejection:0.13), (FOSFOMYCIN:Prophylaxis against transplant rejection:0.45), (FUSIDIC ACID:Prophylaxis against transplant rejection:0.22), (GLYCOPYRRONIUMINDACATEROL:Prophylaxis against transplant rejection:0.52), (HYDROXYCHLOROQUINE DIPHOSPHATE:Prophylaxis against transplant rejection:0.15), (HYOSCYAMINE:Prophylaxis against transplant rejection:0.28), (IDELALISIB:Prophylaxis against transplant rejection:0.74), (ILOPROST:Prophylaxis against transplant rejection:0.34), (IMIPENEM:Prophylaxis against transplant rejection:1.16), (IMIPRAMINE HYDROCHLORIDE:Prophylaxis against transplant rejection:0.02), (INOTUZUMAB OZOGAMICIN:Prophylaxis against transplant rejection:0.29), (INSULIN DEGLUDEC:Prophylaxis against transplant rejection:0.08), (INTERFERON:Prophylaxis against transplant rejection:0.55), (INTERFERON GAMMA-1B:Prophylaxis against transplant rejection:0.15), (ISOFLURANE:Prophylaxis against transplant rejection:0.29), (IXAZOMIB:Prophylaxis against transplant rejection:0.1), (KETOROLACKETOROLAC TROMETHAMINE:Prophylaxis against transplant rejection:0.01), (LEVALBUTEROL HYDROCHLORIDE:Prophylaxis against transplant rejection:0.78), (LEVOFLOXACIN HYDROCHLORIDE:Prophylaxis against transplant rejection:0.89), (LOMITAPIDE MESYLATE:Prophylaxis against transplant rejection:0.02), (LOPINAVIR:Prophylaxis against transplant rejection:0.33), (MEPERIDINE:Prophylaxis against transplant rejection:0.01), (METHENOLONE ENANTHATE:Prophylaxis against transplant rejection:0.07), (MODAFINIL:Prophylaxis against transplant rejection:0.32), (MUPIROCIN:Prophylaxis against transplant rejection:0.16), (MUPIROCINMUPIROCIN CALCIUM:Prophylaxis against transplant rejection:0.38), (NALOXONE:Prophylaxis against transplant rejection:0.01), (NALOXONE HYDROCHLORIDETILIDINE HYDROCHLORIDE:Prophylaxis against transplant rejection:0.23), (NAPROXEN:Prophylaxis against transplant rejection:1.79), (NAPROXEN SODIUM:Prophylaxis against transplant rejection:0.25), (NICORANDIL:Prophylaxis against transplant rejection:0.17), (NOREPINEPHRINE:Prophylaxis against transplant rejection:1.1), (PANTOPRAZOLE MAGNESIUM:Prophylaxis against transplant rejection:0.75), (PAROXETINE:Prophylaxis against transplant rejection:0.75), (PATIROMER:Prophylaxis against transplant rejection:0.17), (PEGASPARGASE:Prophylaxis against transplant rejection:0.41), (PEGFILGRASTIM:Prophylaxis against transplant rejection:0.08), (PEGINTERFERON ALFA-2A OR PEGINTERFERON ALFA-2B:Prophylaxis against transplant rejection:0.04), (PEMETREXED:Prophylaxis against transplant rejection:0.05), (PEMETREXED DISODIUM:Prophylaxis against transplant rejection:0.04), (PHENYTOIN SODIUM:Prophylaxis against transplant rejection:0.74), (PIPERACILLIN:Prophylaxis against transplant rejection:0.13), (PIROXICAM:Prophylaxis against transplant rejection:0.1), (PRAMIPEXOLE DIHYDROCHLORIDE:Prophylaxis against transplant rejection:0.66), (PRAZEPAM:Prophylaxis against transplant rejection:0.14), (PREDNISOLONE SODIUM PHOSPHATE:Prophylaxis against transplant rejection:1.11), (PRISTINAMYCIN:Prophylaxis against transplant rejection:0.07), (PYRIDOSTIGMINE BROMIDE:Prophylaxis against transplant rejection:0.09), (PYRIDOXINE:Prophylaxis against transplant rejection:0.29), (RALOXIFENE HYDROCHLORIDE:Prophylaxis against transplant rejection:0.61), (REGORAFENIB:Prophylaxis against transplant rejection:0.11), (ROFLUMILAST:Prophylaxis against transplant rejection:0.35), (SACUBITRILVALSARTAN:Prophylaxis against transplant rejection:0.33), (SECUKINUMAB:Prophylaxis against transplant rejection:1.11), (SITAGLIPTIN:Prophylaxis against transplant rejection:0.69), (SODIUM CITRATE:Prophylaxis against transplant rejection:0.07), (SUMATRIPTANSUMATRIPTAN SUCCINATE:Prophylaxis against transplant rejection:0.8), (TAZOBACTAM:Prophylaxis against transplant rejection:0.43), (THIOPENTAL SODIUM:Prophylaxis against transplant rejection:0.08), (THROMBOPOIETIN:Prophylaxis against transplant rejection:0.39), (TIMOLOL:Prophylaxis against transplant rejection:1.04), (TINZAPARIN:Prophylaxis against transplant rejection:0.04), (TIZANIDINE:Prophylaxis against transplant rejection:0.37), (TIZANIDINE HYDROCHLORIDE:Prophylaxis against transplant rejection:0.75), (TRANEXAMIC ACID:Prophylaxis against transplant rejection:0.42), (TROSPIUM CHLORIDE:Prophylaxis against transplant rejection:0.01), (UMECLIDINIUM BROMIDEVILANTEROL TRIFENATATE:Prophylaxis against transplant rejection:0.37), (VITAMIN A:Prophylaxis against transplant rejection:0.3), (ZINC:Prophylaxis against transplant rejection:0.36), (ZONISAMIDE:Prophylaxis against transplant rejection:0.19), (.ALPHA.1-PROTEINASE INHIBITOR HUMAN:Prophylaxis of nausea and vomiting:0.44), (ACETAMINOPHENOXYCODONEOXYCODONE HYDROCHLORIDE:Prophylaxis of nausea and vomiting:1.24), (ACETAMINOPHENPROPOXYPHENE HYDROCHLORIDE:Prophylaxis of nausea and vomiting:0.37), (ACETAMINOPHENPROPOXYPHENE NAPSYLATE:Prophylaxis of nausea and vomiting:0.6), (AGALSIDASE BETA:Prophylaxis of nausea and vomiting:0.81), (ALECTINIB:Prophylaxis of nausea and vomiting:0.55), (ALEMTUZUMAB:Prophylaxis of nausea and vomiting:0.78), (ALISKIREN HEMIFUMARATE:Prophylaxis of nausea and vomiting:0.32), (ALUMINUM HYDROXIDEMAGNESIUM OXIDEOXETHAZAINE:Prophylaxis of nausea and vomiting:0.93), (AMANTADINE:Prophylaxis of nausea and vomiting:0.85), (AMANTADINE HYDROCHLORIDE:Prophylaxis of nausea and vomiting:0.45), (AMEZINIUM METILSULFATE:Prophylaxis of nausea and vomiting:0.05), (AMINO ACIDSCARBOHYDRATESMINERALSSOYBEAN OILVITAMINS:Prophylaxis of nausea and vomiting:0.7), (AMISULPRIDE:Prophylaxis of nausea and vomiting:1.11), (AMLODIPINE BESYLATEHYDROCHLOROTHIAZIDEOLMESARTAN MEDOXOMIL:Prophylaxis of nausea and vomiting:0.17), (AMLODIPINE BESYLATEHYDROCHLOROTHIAZIDEVALSARTAN:Prophylaxis of nausea and vomiting:0.23), (AMLODIPINE BESYLATEIRBESARTAN:Prophylaxis of nausea and vomiting:0.18), (AMLODIPINEVALSARTAN:Prophylaxis of nausea and vomiting:0.94), (AMMONIUM GLYCYRRHIZATECYSTEINE HYDROCHLORIDEGLYCINE:Prophylaxis of nausea and vomiting:0.8), (AMOXICILLINCLARITHROMYCINESOMEPRAZOLE:Prophylaxis of nausea and vomiting:0.39), (AMPHOTERICIN BDIMYRISTOYLPHOSPHATIDYLCHOLINE, DL-DIMYRISTOYLPHOSPHATIDYLGLYCEROL, DL-:Prophylaxis of nausea and vomiting:0.12), (AMPICILLIN SODIUM:Prophylaxis of nausea and vomiting:0.4), (ANAGRELIDE HYDROCHLORIDE:Prophylaxis of nausea and vomiting:0.25), (ANTIHISTAMINES NOS:Prophylaxis of nausea and vomiting:0.44), (ARMODAFINIL:Prophylaxis of nausea and vomiting:0.88), (ARSENIC TRIOXIDE:Prophylaxis of nausea and vomiting:0.79), (ASPIRINCLOPIDOGREL:Prophylaxis of nausea and vomiting:0.05), (ASPIRINMAGNESIUM OXIDE:Prophylaxis of nausea and vomiting:0.75), (AZITHROMYCIN OR METHOTREXATE SODIUM:Prophylaxis of nausea and vomiting:0.35), (AZOSEMIDE:Prophylaxis of nausea and vomiting:1.12), (BALSALAZIDE DISODIUM:Prophylaxis of nausea and vomiting:0.07), (BECLOMETHASONE:Prophylaxis of nausea and vomiting:0.45), (BELATACEPT:Prophylaxis of nausea and vomiting:0.2), (BEPRIDIL:Prophylaxis of nausea and vomiting:0.21), (BETAHISTINE MESILATE:Prophylaxis of nausea and vomiting:0.43), (BETAMETHASONE DIPROPIONATECLOTRIMAZOLE:Prophylaxis of nausea and vomiting:0.24), (BETAMETHASONECALCIPOTRIENE:Prophylaxis of nausea and vomiting:0.44), (BETAMIPRONPANIPENEM:Prophylaxis of nausea and vomiting:0.97), (BETHANECHOL CHLORIDE:Prophylaxis of nausea and vomiting:0.29), (BIAPENEM:Prophylaxis of nausea and vomiting:0.54), (LONGUM:Prophylaxis of nausea and vomiting:0.06), (BLESELUMAB:Prophylaxis of nausea and vomiting:0.2), (BRINZOLAMIDE:Prophylaxis of nausea and vomiting:1.07), (BROMHEXINE:Prophylaxis of nausea and vomiting:0.05), (BUCILLAMINE:Prophylaxis of nausea and vomiting:0.65), (BUPRENORPHINE HYDROCHLORIDENALOXONE HYDROCHLORIDE:Prophylaxis of nausea and vomiting:0.2), (CABOZANTINIB:Prophylaxis of nausea and vomiting:0.21), (CABOZANTINIB S-MALATE:Prophylaxis of nausea and vomiting:0.2), (CALCITONIN:Prophylaxis of nausea and vomiting:0.95), (CALCIUM CARBONATEERGOCALCIFEROLRETINOL:Prophylaxis of nausea and vomiting:0.54), (CANAGLIFLOZIN:Prophylaxis of nausea and vomiting:0.72), (CANAKINUMAB:Prophylaxis of nausea and vomiting:0.72), (CAPSAICIN:Prophylaxis of nausea and vomiting:0.04), (CARBIDOPA:Prophylaxis of nausea and vomiting:0.01), (CARVEDILOL PHOSPHATE:Prophylaxis of nausea and vomiting:1.23), (CEFOTAXIMECEFOTAXIME SODIUM:Prophylaxis of nausea and vomiting:1.27), (CEFTAZIDIME SODIUM:Prophylaxis of nausea and vomiting:0.08), (CERTOLIZUMAB PEGOL:Prophylaxis of nausea and vomiting:1.44), (CHIMAPHILA UMBELLATAMANGANESE CHLORIDETREMULOIDESSODIUM TAUROCHOLATE:Prophylaxis of nausea and vomiting:0.2), (CHLORMADINONE ACETATE:Prophylaxis of nausea and vomiting:0.03), (CHLOROTHIAZIDE:Prophylaxis of nausea and vomiting:0.06), (CHONDROITIN SULFATE (BOVINE)GLUCOSAMINE HYDROCHLORIDE:Prophylaxis of nausea and vomiting:0.47), (CIPROFIBRATE:Prophylaxis of nausea and vomiting:0.53), (CLADRIBINE:Prophylaxis of nausea and vomiting:0.9), (CLOMIPRAMINE HYDROCHLORIDE:Prophylaxis of nausea and vomiting:0.77), (CLOXACILLIN SODIUM:Prophylaxis of nausea and vomiting:0.13), (CODEINE PHOSPHATEGUAIFENESIN:Prophylaxis of nausea and vomiting:0.19), (COLCHICINEDICYCLOMINE HYDROCHLORIDE:Prophylaxis of nausea and vomiting:0.18), (COLISTIN:Prophylaxis of nausea and vomiting:0.97), (CYCLOPHOSPHAMIDECYTARABINEDEXAMETHASONEDOXORUBI CINMETHOTREXATEVINCRISTINE:Prophylaxis of nausea and vomiting:0.56), (DABIGATRAN:Prophylaxis of nausea and vomiting:1.05), (DABRAFENIB MESYLATE:Prophylaxis of nausea and vomiting:0.85), (DACLIZUMAB:Prophylaxis of nausea and vomiting:1.48), (DANAZOL:Prophylaxis of nausea and vomiting:1.18), (DAPAGLIFLOZIN PROPANEDIOL:Prophylaxis of nausea and vomiting:0.14), (DEGARELIX:Prophylaxis of nausea and vomiting:0.53), (DESMOPRESSIN:Prophylaxis of nausea and vomiting:0.54), (DESOGESTRELETHINYL ESTRADIOL:Prophylaxis of nausea and vomiting:0.04), (DESVENLAFAXINE SUCCINATE:Prophylaxis of nausea and vomiting:0.32), (DEVICE:Prophylaxis of nausea and vomiting:0.31), (DINUTUXIMAB:Prophylaxis of nausea and vomiting:0.25), (DIPYRIDAMOLE:Prophylaxis of nausea and vomiting:1.43), (DOBUTAMINE HYDROCHLORIDE:Prophylaxis of nausea and vomiting:0.89), (DOFETILIDE:Prophylaxis of nausea and vomiting:0.76), (DONEPEZIL HYDROCHLORIDE:Prophylaxis of nausea and vomiting:0.09), (DORZOLAMIDE:Prophylaxis of nausea and vomiting:0.44), (DRONEDARONE:Prophylaxis of nausea and vomiting:0.51), (ECONAZOLE:Prophylaxis of nausea and vomiting:1.56), (EDOXABAN TOSYLATE:Prophylaxis of nausea and vomiting:1.92), (EMPAGLIFLOZINLINAGLIPTIN:Prophylaxis of nausea and vomiting:0.43), (EPACADOSTAT:Prophylaxis of nausea and vomiting:0.37), (EPERISONE HYDROCHLORIDE:Prophylaxis of nausea and vomiting:0.21), (ERTAPENEM:Prophylaxis of nausea and vomiting:0.34), (ESTROGENS, CONJUGATEDMEDROXYPROGESTERONE ACETATE:Prophylaxis of nausea and vomiting:0.42), (ESTRONESUS SCROFA OVARY:Prophylaxis of nausea and vomiting:0.21), (ETHINYL ESTRADIOLNORGESTREL:Prophylaxis of nausea and vomiting:0.01), (ETHYL ICOSAPENTATEICOSAPENT:Prophylaxis of nausea and vomiting:0.11), (ETIMICIN SULFATE:Prophylaxis of nausea and vomiting:0.15), (ETOMIDATE:Prophylaxis of nausea and vomiting:0.51), (ETRETINATE:Prophylaxis of nausea and vomiting:1.41), (EVOLOCUMAB:Prophylaxis of nausea and vomiting:0.37), (FAROPENEM SODIUM:Prophylaxis of nausea and vomiting:0.23), (FERROUS SULFATEGLYCINE:Prophylaxis of nausea and vomiting:0.7), (FIBRINOGEN HUMAN:Prophylaxis of nausea and vomiting:0.08), (FINGOLIMOD HYDROCHLORIDE:Prophylaxis of nausea and vomiting:0.92), (FLUCYTOSINE:Prophylaxis of nausea and vomiting:0.45), (FLUOCINOLONE ACETONIDE:Prophylaxis of nausea and vomiting:0.03), (FLUTAMIDE:Prophylaxis of nausea and vomiting:0.4), (FLUTICASONE PROPIONATEFORMOTEROL FUMARATE:Prophylaxis of nausea and vomiting:0.2), (FOSFLUCONAZOLE:Prophylaxis of nausea and vomiting:0.91), (FUDOSTEINE:Prophylaxis of nausea and vomiting:0.03), (GARENOXACIN:Prophylaxis of nausea and vomiting:0.12), (GLYCOPYRROLATEINDACATEROL MALEATE:Prophylaxis of nausea and vomiting:0.33), (HERBALSTURMERIC:Prophylaxis of nausea and vomiting:0.36), (HYDROCHLOROTHIAZIDERAMIPRIL:Prophylaxis of nausea and vomiting:0.14), (HYDROCHLOROTHIAZIDETELMISARTAN:Prophylaxis of nausea and vomiting:1.27), (HYOSCYAMINE:Prophylaxis of nausea and vomiting:0.67), (ICOSAPENT ETHYL:Prophylaxis of nausea and vomiting:0.13), (IGURATIMOD:Prophylaxis of nausea and vomiting:0.08), (ILOPROST:Prophylaxis of nausea and vomiting:0.4), (IMIGLUCERASE:Prophylaxis of nausea and vomiting:0.2), (IMIPRAMINE HYDROCHLORIDE:Prophylaxis of nausea and vomiting:0.75), (IMMUNE GLOBULIN NOS:Prophylaxis of nausea and vomiting:0.63), (INFLIXIMAB-DYYB:Prophylaxis of nausea and vomiting:0.33), (INTERFERON ALFA-2A:Prophylaxis of nausea and vomiting:1.53), (INTERFERON ALFA-2A OR INTERFERON ALFA-2B:Prophylaxis of nausea and vomiting:0.37), (INTERFERON ALFA-2B:Prophylaxis of nausea and vomiting:1.55), (INTERFERON GAMMA-1B:Prophylaxis of nausea and vomiting:0.58), (ISONIAZIDPYRAZINAMIDERIFAMPIN:Prophylaxis of nausea and vomiting:0.14), (ISONIAZIDRIFAMPIN:Prophylaxis of nausea and vomiting:0.07), (ISOSORBIDE:Prophylaxis of nausea and vomiting:1.24), (IXABEPILONE:Prophylaxis of nausea and vomiting:0.37), (LACTASE:Prophylaxis of nausea and vomiting:0.25), (ACIDOPHILUSLACTOBACILLUS DELBRUECKII SUBSP. BULGARICUS:Prophylaxis of nausea and vomiting:0.12), (LACTOBACILLUS RHAMNOSUS:Prophylaxis of nausea and vomiting:0.99), (LANREOTIDE:Prophylaxis of nausea and vomiting:0.25), (LANREOTIDE ACETATE:Prophylaxis of nausea and vomiting:0.32), (LAPATINIB:Prophylaxis of nausea and vomiting:0.74), (LEUCOGEN:Prophylaxis of nausea and vomiting:0.24), (LEVOBUPIVACAINE:Prophylaxis of nausea and vomiting:0.05), (LEVOCARNITINE HYDROCHLORIDE:Prophylaxis of nausea and vomiting:0.14), (LEVOTHYROXINE SODIUMPOTASSIUM IODIDE:Prophylaxis of nausea and vomiting:0.3), (LIOTHYRONINE SODIUM:Prophylaxis of nausea and vomiting:0.29), (LISDEXAMFETAMINE DIMESYLATE:Prophylaxis of nausea and vomiting:0.21), (LITHIUM:Prophylaxis of nausea and vomiting:0.14), (MAGNESIUM ASPARTATE HYDROCHLORIDE:Prophylaxis of nausea and vomiting:0.59), (MAGNESIUM ASPARTATE\POTASSIUM ASPARTATE:Prophylaxis of nausea and vomiting:0.68), (MAGNESIUM PIDOLATE:Prophylaxis of nausea and vomiting:0.2), (MAXACALCITOL:Prophylaxis of nausea and vomiting:0.17), (MEDROXYPROGESTERONE:Prophylaxis of nausea and vomiting:0.02), (MELPERONE:Prophylaxis of nausea and vomiting:0.45), (MEPOLIZUMAB:Prophylaxis of nausea and vomiting:0.36), (METAXALONE:Prophylaxis of nausea and vomiting:1.01), (METFORMIN HYDROCHLORIDESAXAGLIPTIN:Prophylaxis of nausea and vomiting:0.14), (METHENOLONE ENANTHATE:Prophylaxis of nausea and vomiting:1.97), (METHYLNALTREXONE:Prophylaxis of nausea and vomiting:0.22), (MEXILETINE HYDROCHLORIDE:Prophylaxis of nausea and vomiting:0.33), (MIDODRINE HYDROCHLORIDE:Prophylaxis of nausea and vomiting:0.31), (MIZORIBINE:Prophylaxis of nausea and vomiting:0.75), (MOGAMULIZUMAB:Prophylaxis of nausea and vomiting:0.46), (NABUMETONE:Prophylaxis of nausea and vomiting:0.52), (NORTRIPTYLINE HYDROCHLORIDE:Prophylaxis of nausea and vomiting:0.05), (OK-432:Prophylaxis of nausea and vomiting:0.23), (ONABOTULINUMTOXINA:Prophylaxis of nausea and vomiting:0.02), (OPIPRAMOL:Prophylaxis of nausea and vomiting:0.19), (ORPHENADRINE:Prophylaxis of nausea and vomiting:0.22), (PANTHENOL:Prophylaxis of nausea and vomiting:0.12), (PARICALCITOL:Prophylaxis of nausea and vomiting:0.04), (PAZUFLOXACIN MESILATE:Prophylaxis of nausea and vomiting:0.4), (PEGINTERFERON ALFA-2B:Prophylaxis of nausea and vomiting:0.66), (PEGINTERFERON BETA-1A:Prophylaxis of nausea and vomiting:0.57), (PENICILLIN G:Prophylaxis of nausea and vomiting:0.74), (PENICILLIN V POTASSIUM:Prophylaxis of nausea and vomiting:0.1), (PENTOBARBITAL:Prophylaxis of nausea and vomiting:0.05), (PHENOBARBITAL SODIUM:Prophylaxis of nausea and vomiting:0.36), (PILSICAINIDE HYDROCHLORIDE:Prophylaxis of nausea and vomiting:0.56), (PIMOBENDAN:Prophylaxis of nausea and vomiting:0.42), (PIROXICAM:Prophylaxis of nausea and vomiting:0.62), (POTASSIUM CITRATETRISODIUM CITRATE DIHYDRATE:Prophylaxis of nausea and vomiting:1.03), (POTASSIUM PHOSPHATE\SODIUM PHOSPHATE:Prophylaxis of nausea and vomiting:0.12), (PRALATREXATE:Prophylaxis of nausea and vomiting:0.01), (PRISTINAMYCIN:Prophylaxis of nausea and vomiting:0.75), (PROCARBAZINE:Prophylaxis of nausea and vomiting:0.2), (PROPAFENONE:Prophylaxis of nausea and vomiting:0.45), (PROPIVERINE:Prophylaxis of nausea and vomiting:0.16), (PYRIMETHAMINE:Prophylaxis of nausea and vomiting:0.21), (RALTITREXED:Prophylaxis of nausea and vomiting:0.32), (RANIMUSTINE:Prophylaxis of nausea and vomiting:0.77), (RILMENIDINE PHOSPHATE:Prophylaxis of nausea and vomiting:0.52), (RILOTUMUMAB:Prophylaxis of nausea and vomiting:0.05), (RIVASTIGMINE TARTRATE:Prophylaxis of nausea and vomiting:1.17), (ROFLUMILAST:Prophylaxis of nausea and vomiting:1.47), (SARGRAMOSTIM:Prophylaxis of nausea and vomiting:0.1), (SECUKINUMAB:Prophylaxis of nausea and vomiting:0.96), (SIPULEUCEL-T:Prophylaxis of nausea and vomiting:0.26), (SITAFLOXACIN:Prophylaxis of nausea and vomiting:0.03), (SODIUM FLUORIDE:Prophylaxis of nausea and vomiting:0.36), (SOLIFENACIN:Prophylaxis of nausea and vomiting:0.21), (SOMATOSTATIN:Prophylaxis of nausea and vomiting:0.18), (SORBITOL:Prophylaxis of nausea and vomiting:0.06), (SOYBEAN OIL:Prophylaxis of nausea and vomiting:0.02), (STREPTOMYCIN\STREPTOMYCIN SULFATE:Prophylaxis of nausea and vomiting:0.23), (SUPLATAST TOSILATE:Prophylaxis of nausea and vomiting:0.38), (TABALUMAB:Prophylaxis of nausea and vomiting:0.15), (TECELEUKIN:Prophylaxis of nausea and vomiting:0.2), (TEDUGLUTIDE\WATER:Prophylaxis of nausea and vomiting:0.44), (TELOTRISTAT ETHYL:Prophylaxis of nausea and vomiting:0.06), (TENIPOSIDE:Prophylaxis of nausea and vomiting:0.24), (TERBUTALINE:Prophylaxis of nausea and vomiting:1.34), (TERIFLUNOMIDE:Prophylaxis of nausea and vomiting:0.12), (TESTOSTERONE UNDECANOATE:Prophylaxis of nausea and vomiting:0.57), (TETRACAINE HYDROCHLORIDE:Prophylaxis of nausea and vomiting:0.02), (TETRACYCLINE:Prophylaxis of nausea and vomiting:1.18), (THEOPHYLLINE:Prophylaxis of nausea and vomiting:0.32), (THIOPENTAL:Prophylaxis of nausea and vomiting:1.22), (THROMBOPOIETIN:Prophylaxis of nausea and vomiting:0.34), (THYROID, PORCINE:Prophylaxis of nausea and vomiting:0.1), (TICLOPIDINE:Prophylaxis of nausea and vomiting:0.41), (TILIDINE:Prophylaxis of nausea and vomiting:0.21), (TIMOLOL MALEATE:Prophylaxis of nausea and vomiting:0.72), (TISAGENLECLEUCEL:Prophylaxis of nausea and vomiting:0.59), (TOLTERODINE TARTRATE:Prophylaxis of nausea and vomiting:0.82), (TRANDOLAPRIL:Prophylaxis of nausea and vomiting:0.44), (TRIHEXYPHENIDYL:Prophylaxis of nausea and vomiting:0.11), (TRIMIPRAMINE MALEATE:Prophylaxis of nausea and vomiting:0.61), (TRIPTORELIN PAMOATE:Prophylaxis of nausea and vomiting:0.08), (TULOBUTEROL:Prophylaxis of nausea and vomiting:0.22), (VADASTUXIMAB TALIRINE:Prophylaxis of nausea and vomiting:0.05), (VANDETANIB:Prophylaxis of nausea and vomiting:0.23), (VILAZODONE HYDROCHLORIDE:Prophylaxis of nausea and vomiting:0.01), (VONOPRAZAN:Prophylaxis of nausea and vomiting:0.04), (ZAFIRLUKAST:Prophylaxis of nausea and vomiting:0.11), (ZIV-AFLIBERCEPT:Prophylaxis of nausea and vomiting:0.96), (DIVALPROEX SODIUM:Prophylaxis ofneural tube defect:0.15), (ACETAZOLAMIDE:Prophylaxis urinary tract infection:0.04), (ALENDRONIC ACID:Prophylaxis urinary tract infection:0.11), (AMIODARONE HYDROCHLORIDE:Prophylaxis urinary tract infection:0.54), (AZITHROMYCIN ANHYDROUS:Prophylaxis urinary tract infection:0.11), (BRINZOLAMIDE:Prophylaxis urinary tract infection:0.02), (BUDESONIDE:Prophylaxis urinary tract infection:1.38), (CALCITRIOL:Prophylaxis urinary tract infection:0.42), (CANDESARTAN CILEXETILHYDROCHLOROTHIAZIDE:Prophylaxis urinary tract infection:0.26), (CARVEDILOL PHOSPHATE:Prophylaxis urinary tract infection:0.07), (CERTOLIZUMAB PEGOL:Prophylaxis urinary tract infection:0.96), (CHOLESTYRAMINE:Prophylaxis urinary tract infection:0.48), (CLINDAMYCINCLINDAMYCIN PHOSPHATE:Prophylaxis urinary tract infection:1.2), (CLONIDINE:Prophylaxis urinary tract infection:0.85), (DACLIZUMAB:Prophylaxis urinary tract infection:0.46), (DASATINIB:Prophylaxis urinary tract infection:0.26), (DICLOFENAC:Prophylaxis urinary tract infection:0.84), (DIMENHYDRINATE:Prophylaxis urinary tract infection:0.08), (DUTASTERIDE:Prophylaxis urinary tract infection:0.3), (EPINEPHRINE:Prophylaxis urinary tract infection:0.55), (EVOLOCUMAB:Prophylaxis urinary tract infection:0.99), (FLECAINIDE:Prophylaxis urinary tract infection:0.21), (GLYBURIDE:Prophylaxis urinary tract infection:0.04), (HERBALSTURMERIC:Prophylaxis urinary tract infection:0.07), (HYDROCHLOROTHIAZIDEIRBESARTAN:Prophylaxis urinary tract infection:0.17), (HYDROCHLOROTHIAZIDELISINOPRIL:Prophylaxis urinary tract infection:0.68), (HYDROCHLOROTHIAZIDELOSARTAN POTASSIUM:Prophylaxis urinary tract infection:0.21), (INDOMETHACIN:Prophylaxis urinary tract infection:0.17), (LEDIPASVIRSOFOSBUVIR:Prophylaxis urinary tract infection:0.23), (LERCANIDIPINE:Prophylaxis urinary tract infection:0.73), (LISDEXAMFETAMINE DIMESYLATE:Prophylaxis urinary tract infection:0.35), (LITHIUM CARBONATE:Prophylaxis urinary tract infection:0.61), (METHYLPREDNISOLONE ACETATE:Prophylaxis urinary tract infection:0.59), (METRONIDAZOLEMETRONIDAZOLE HYDROCHLORIDE:Prophylaxis urinary tract infection:0.42), (MOXIFLOXACIN HYDROCHLORIDE:Prophylaxis urinary tract infection:0.95), (NILOTINIB:Prophylaxis urinary tract infection:0.51), (OMEGA-3-ACID ETHYL ESTERS:Prophylaxis urinary tract infection:0.36), (OXYGEN:Prophylaxis urinary tract infection:0.39), (PAROXETINE:Prophylaxis urinary tract infection:0.35), (PAROXETINEPAROXETINE HYDROCHLORIDE:Prophylaxis urinary tract infection:0.37), (PENICILLIN:Prophylaxis urinary tract infection:0.35), (PHENOBARBITAL:Prophylaxis urinary tract infection:0.2), (PHENYTOIN:Prophylaxis urinary tract infection:0.16), (PRAMIPEXOLE DIHYDROCHLORIDE:Prophylaxis urinary tract infection:0.88), (RABEPRAZOLE:Prophylaxis urinary tract infection:0.24), (RABEPRAZOLE SODIUM:Prophylaxis urinary tract infection:0.44), (RANOLAZINE:Prophylaxis urinary tract infection:1.06), (RIZATRIPTAN BENZOATE:Prophylaxis urinary tract infection:0.19), (ROFLUMILAST:Prophylaxis urinary tract infection:0.18), (SACUBITRILVALSARTAN:Prophylaxis urinary tract infection:0.98), (SECUKINUMAB:Prophylaxis urinary tract infection:0.33), (SOTALOL:Prophylaxis urinary tract infection:0.42), (SUMATRIPTAN:Prophylaxis urinary tract infection:0.17), (SUMATRIPTANSUMATRIPTAN SUCCINATE:Prophylaxis urinary tract infection:0.1), (TOLTERODINE TARTRATE:Prophylaxis urinary tract infection:0.47), (TRIAMTERENE:Prophylaxis urinary tract infection:0.31), (UMECLIDINIUM BROMIDEVILANTEROL TRIFENATATE:Prophylaxis urinary tract infection:0.34), (VARENICLINE TARTRATE:Prophylaxis urinary tract infection:0.96), (ACETAMINOPHENPROPOXYPHENE HYDROCHLORIDE:Prostate cancer:0.55), (ACETAMINOPHENPROPOXYPHENE NAPSYLATE:Prostate cancer:0.86), (ADENINE:Prostate cancer:0.35), (ADO-TRASTUZUMAB EMTANSINE:Prostate cancer:2.72), (AFATINIB:Prostate cancer:1.05), (AGALSIDASE BETA:Prostate cancer:1.63), (ALBIGLUTIDE:Prostate cancer:0.16), (ALDESLEUKIN:Prostate cancer:0.7), (ALECTINIB:Prostate cancer:0.15), (ALEMTUZUMAB:Prostate cancer:0.42), (ALUMINUM HYDROXIDE:Prostate cancer:0.09), (ALUMINUM HYDROXIDEMAGNESIUM HYDROXIDESIMETHICONE:Prostate cancer:0.16), (AMINO ACIDSCARBOHYDRATESMINERALSSOYBEAN OILVITAMINS:Prostate cancer:0), (AMINO ACIDSDEXTROSEELECTROLYTES NOS:Prostate cancer:0.97), (AMINO ACIDSDEXTROSEELECTROLYTES NOSSOYBEAN OIL:Prostate cancer:0.02), (AMISULPRIDE:Prostate cancer:1.52), (AMLODIPINE BESYLATECANDESARTAN CILEXETIL:Prostate cancer:0), (AMLODIPINE BESYLATEHYDROCHLOROTHIAZIDEVALSARTAN:Prostate cancer:1.84), (AMOXAPINE:Prostate cancer:0.38), (AMOXICILLINCLARITHROMYCINESOMEPRAZOLE:Prostate cancer:0.47), (AMPICILLIN SODIUM:Prostate cancer:0.39), (ARIPIPRAZOLE LAUROXIL:Prostate cancer:0.16), (ASCORBIC ACIDBIOTINCYANOCOBALAMINFOLIC ACIDNIACINPANTOTHENIC ACIDPYRIDOXINE\RIBOFLAVINTHIAMINE:Prostate cancer:0.38), (ASPIRINCLOPIDOGREL:Prostate cancer:0.71), (ATORVASTATIN CALCIUMEZETIMIBE:Prostate cancer:0.2), (ATROPINE SULFATE:Prostate cancer:0.63), (ATROPINE SULFATEHYOSCYAMINE SULFATEPHENOBARBITALSCOPOLAMINE HYDROBROMIDE:Prostate cancer:0.12), (AZITHROMYCIN OR METHOTREXATE SODIUM:Prostate cancer:0.1), (BAZEDOXIFENE ACETATEESTROGENS, CONJUGATED:Prostate cancer:0.08), (BECLOMETHASONE DIPROPIONATEFORMOTEROL:Prostate cancer:0.2), (BENSERAZIDELEVODOPA:Prostate cancer:0.27), (BENZOCAINE:Prostate cancer:0.05), (BENZTROPINE MESYLATE:Prostate cancer:0.66), (BEPOTASTINE:Prostate cancer:0.32), (BETAMETHASONE DIPROPIONATECALCIPOTRIENE:Prostate cancer:0.36), (BETAMETHASONE DIPROPIONATECALCIPOTRIENECALCIPOTRIENE HYDRATE:Prostate cancer:0.78), (BLINATUMOMAB:Prostate cancer:0.26), (BOCEPREVIR:Prostate cancer:0.18), (BOCOCIZUMAB:Prostate cancer:0.75), (BOSUTINIB:Prostate cancer:0.38), (BOTULINUM TOXIN TYPE A:Prostate cancer:0.24), (CALCIUM CHLORIDE ANHYDROUSPOTASSIUM CHLORIDE\SODIUM CHLORIDE\SODIUM LACTATE:Prostate cancer:0.18), (CALCIUM PANTOTHENATECYANOCOBALAMINNIACINAMIDEPYRIDOXINE HYDROCHLORIDE\RIBOFLAVINTHIAMINE MONONITRATE:Prostate cancer:0.15), (CALCIUM\MAGNESIUM\ZINC:Prostate cancer:0.86), (CANAGLIFLOZINMETFORMIN HYDROCHLORIDE:Prostate cancer:1.42), (CAPSAICIN:Prostate cancer:0.35), (CARBOHYDRATESFATTY ACIDSMINERALSPROTEINVITAMINS:Prostate cancer:0.1), (CARTEOLOL:Prostate cancer:0.32), (CEFDITOREN PIVOXIL:Prostate cancer:0.26), (CEFEPIMECEFEPIME HYDROCHLORIDE:Prostate cancer:0.82), (CEFPODOXIME PROXETIL:Prostate cancer:0.71), (CERIVASTATIN SODIUM:Prostate cancer:0.1), (CEVIMELINE HYDROCHLORIDE:Prostate cancer:0.53), (CHLOROTHIAZIDE:Prostate cancer:0.12), (CHLORPROTHIXENE:Prostate cancer:0.02), (CHOLESTEROL:Prostate cancer:0.23), (CHONDROITIN SULFATE (BOVINE):Prostate cancer:0.05), (CLIKSTAR:Prostate cancer:0.59), (CYAMEMAZINE:Prostate cancer:0.47), (CYANOCOBALAMINPYRIDOXINETHIAMINE:Prostate cancer:0.11), (DACLATASVIR:Prostate cancer:0.12), (DACLIZUMAB:Prostate cancer:0.92), (DANAZOL:Prostate cancer:1.39), (DAPAGLIFLOZIN PROPANEDIOL:Prostate cancer:1.15), (DAPAGLIFLOZIN PROPANEDIOLMETFORMIN HYDROCHLORIDE:Prostate cancer:0.24), (DEXTROAMPHETAMINE SULFATE:Prostate cancer:0.91), (DIFLUNISAL:Prostate cancer:0.46), (DIGITALIS:Prostate cancer:0.16), (DILAZEP:Prostate cancer:0.19), (DIPYRONEPROMETHAZINE HYDROCHLORIDE:Prostate cancer:0.66), (DOBUTAMINE HYDROCHLORIDE:Prostate cancer:0.16), (EMPAGLIFLOZINLINAGLIPTIN:Prostate cancer:0.77), (ENSULIZOLEEUCERINOCTINOXATEOCTISALATETITANIUM DIOXIDE\ZINC OXIDE:Prostate cancer:0.08), (ERIBULIN:Prostate cancer:1.56), (ERIBULIN MESYLATE:Prostate cancer:0.65), (ERYTHROMYCINSULFISOXAZOLE:Prostate cancer:0.18), (ESOMEPRAZOLE SODIUM:Prostate cancer:0.24), (ESTRADIOLNORETHINDRONE ACETATE:Prostate cancer:0.19), (ESTRIOL:Prostate cancer:0.33), (ESTROGENS, CONJUGATEDMEDROXYPROGESTERONE ACETATE:Prostate cancer:1.34), (ETHACRYNIC ACID:Prostate cancer:0.3), (ETRETINATE:Prostate cancer:1.4), (FENOTEROL HYDROBROMIDE:Prostate cancer:1.27), (FERRIC HYDROXIDE:Prostate cancer:1.09), (FERRIC OXYHYDROXIDE:Prostate cancer:0.17), (FERROUS BISGLYCINATE:Prostate cancer:0.23), (FERROUS SULFATEGLYCINE:Prostate cancer:0.99), (FLUNARIZINE HYDROCHLORIDE:Prostate cancer:0.19), (FLURBIPROFEN:Prostate cancer:0.53), (FOSAPREPITANT DIMEGLUMINE:Prostate cancer:0.03), (FULVESTRANT:Prostate cancer:1.7), (GADOVERSETAMIDE:Prostate cancer:0.65), (GEFITINIB:Prostate cancer:1.17), (GLYCERINHYPROMELLOSESPOLYETHYLENE GLYCOL 400:Prostate cancer:0.28), (GLYCOPYRROLATEINDACATEROL MALEATE:Prostate cancer:0.82), (GUANABENZ ACETATE:Prostate cancer:0.16), (HUMAN PLATELET, ALLOGENIC:Prostate cancer:0.04), (HYDROCHLOROTHIAZIDEMETOPROLOL TARTRATE:Prostate cancer:0.02), (IDARUCIZUMAB:Prostate cancer:0.57), (IGURATIMOD:Prostate cancer:0.38), (ILOPROST:Prostate cancer:1.23), (IMIGLUCERASE:Prostate cancer:0.5), (IMMUNE GLOBULIN NOS:Prostate cancer:0.25), (INFLIXIMAB-DYYB:Prostate cancer:0.38), (INSULIN ASPARTINSULIN DEGLUDEC:Prostate cancer:0.56), (INSULIN PORKANSULIN PURIFIED PORK:Prostate cancer:0.35), (INTERFERON:Prostate cancer:0.84), (INTERFERON ALFA-2A:Prostate cancer:1.34), (INTERFERON ALFA-2A OR INTERFERON ALFA-2B:Prostate cancer:0.12), (INTERFERON ALFA-2B:Prostate cancer:1.3), (INTERFERON GAMMA-1B:Prostate cancer:0.14), (ITRACONAZOLE\SODIUM CHLORIDE:Prostate cancer:0.07), (LAPATINIB:Prostate cancer:0.7), (LAPATINIB DITOSYLATE:Prostate cancer:1.54), (LENVATINIB:Prostate cancer:0.87), (LEVOCABASTINE HYDROCHLORIDE:Prostate cancer:0.21), (LEVOMEFOLATE CALCIUM:Prostate cancer:0.49), (LEVOMILNACIPRAN HYDROCHLORIDE:Prostate cancer:0.14), (LEVOTHYROXINE SODIUMPOTASSIUM IODIDE:Prostate cancer:0.45), (LINEZOLID:Prostate cancer:0.48), (LINSIDOMINE:Prostate cancer:0.69), (LOMITAPIDE MESYLATE:Prostate cancer:1.03), (LUTETIUM LU-177:Prostate cancer:0.63), (MAGNESIUM ASPARTATE HYDROCHLORIDE:Prostate cancer:0.55), (MAGNESIUM PIDOLATE:Prostate cancer:0.42), (MAXACALCITOL:Prostate cancer:0.66), (MEPOLIZUMAB:Prostate cancer:0.88), (METFORMIN HYDROCHLORIDEROSIGLITAZONE MALEATE:Prostate cancer:0.83), (METFORMIN HYDROCHLORIDESAXAGLIPTIN:Prostate cancer:0.6), (METHENAMINE MANDELATE:Prostate cancer:0.15), (METHENOLONE ENANTHATE:Prostate cancer:0.61), (MIZORIBINE:Prostate cancer:0.34), (NARATRIPTAN HYDROCHLORIDE:Prostate cancer:0.33), (NIACINAMIDE:Prostate cancer:0.18), (NINTEDANIB:Prostate cancer:1.72), (OLOPATADINE:Prostate cancer:0.12), (OMEPRAZOLE\SODIUM BICARBONATE:Prostate cancer:0.23), (OPIPRAMOL:Prostate cancer:0.74), (ORPHENADRINE:Prostate cancer:1.02), (OSELTAMIVIR:Prostate cancer:0.43), (OSPEMIFENE:Prostate cancer:0.25), (OXYBUTYNINOXYBUTYNIN CHLORIDE:Prostate cancer:0.91), (PALIPERIDONE PALMITATE:Prostate cancer:0.43), (PEGINTERFERON BETA-1A:Prostate cancer:1.23), (PEGVISOMANT:Prostate cancer:0.15), (PENTAMIDINE ISETHIONATE:Prostate cancer:0.02), (PERPHENAZINE:Prostate cancer:0.16), (PERTUZUMAB:Prostate cancer:1.6), (PILOCARPINE HYDROCHLORIDE:Prostate cancer:0.48), (PIMECROLIMUS:Prostate cancer:0.26), (PIMOBENDAN:Prostate cancer:1.12), (PIRFENIDONE:Prostate cancer:0.48), (PIRITRAMIDE:Prostate cancer:0.36), (POLYSTYRENE:Prostate cancer:0.5), (POTASSIUM CITRATETRISODIUM CITRATE DIHYDRATE:Prostate cancer:0.42), (PRASUGREL:Prostate cancer:0.35), (PREDNISOLONE SODIUM SUCCINATE:Prostate cancer:0.03), (PROPATYL NITRATE:Prostate cancer:0.22), (PROTRIPTYLINE HYDROCHLORIDE:Prostate cancer:0.47), (PRUCALOPRIDE SUCCINATE:Prostate cancer:0.13), (PYRIDOXAL:Prostate cancer:0.42), (PYRIDOXAL PHOSPHATE:Prostate cancer:0.13), (QUINACRINE:Prostate cancer:0.08), (RALOXIFENE HYDROCHLORIDE:Prostate cancer:1.4), (RAMUCIRUMAB:Prostate cancer:1.59), (RIBOCICLIB:Prostate cancer:0.01), (RILPIVIRINE HYDROCHLORIDE:Prostate cancer:0.19), (RISEDRONIC ACID:Prostate cancer:0.13), (RUCAPARIB CAMSYLATE:Prostate cancer:0.59), (SARPOGRELATE HYDROCHLORIDE:Prostate cancer:0.06), (SECUKINUMAB:Prostate cancer:1.5), (SELEXIPAG:Prostate cancer:0.32), (SIBUTRAMINE HYDROCHLORIDE:Prostate cancer:0.1), (SOFOSBUVIRVELPATASVIR:Prostate cancer:0.29), (SOMATOSTATIN:Prostate cancer:0.06), (STARCH, WHEAT:Prostate cancer:0.94), (SUMATRIPTANSUMATRIPTAN SUCCINATE:Prostate cancer:1.65), (SUPLATAST TOSILATE:Prostate cancer:0.18), (TEDUGLUTIDE\WATER:Prostate cancer:0.49), (TELOTRISTAT ETHYL:Prostate cancer:0.54), (TERBUTALINE SULFATE:Prostate cancer:2.23), (TESTOSTERONE ENANTHATE:Prostate cancer:0.1), (TESTOSTERONE UNDECANOATE:Prostate cancer:0.87), (THEOPHYLLINE:Prostate cancer:0.38), (THYROID, PORCINETHYROID, UNSPECIFIED:Prostate cancer:0.34), (TIBOLONE:Prostate cancer:0.24), (TIMOLOL MALEATETRAVOPROST:Prostate cancer:1.21), (TOLVAPTAN:Prostate cancer:0.64), (TOREMIFENE:Prostate cancer:0.1), (TRABECTEDIN:Prostate cancer:0.24), (TRAMETINIB:Prostate cancer:1.37), (TRIHEXYPHENIDYL:Prostate cancer:0.36), (TRIMIPRAMINE MALEATE:Prostate cancer:0.91), (TROXIPIDE:Prostate cancer:0.04), (UROKINASE:Prostate cancer:0.21), (VALDECOXIB:Prostate cancer:0.79), (VALGANCICLOVIR HYDROCHLORIDE:Prostate cancer:1.54), (VASOPRESSIN:Prostate cancer:0.1), (VORAPAXAR SULFATE:Prostate cancer:0.05), (XIPAMIDE:Prostate cancer:0.62), (YEAST:Prostate cancer:0.41), (ZILEUTON:Prostate cancer:0.83), (ZIPRASIDONE HYDROCHLORIDEZIPRASIDONE MESYLATE:Prostate cancer:0.24), (ZOLMITRIPTAN:Prostate cancer:0.54), (ACARBOSE:Prostate cancer metastatic:0.51), (ACETAMINOPHENBUTALBITALCAFFEINE:Prostate cancer metastatic:0.86), (ACETAZOLAMIDE:Prostate cancer metastatic:0.76), (ACETYLCYSTEINE:Prostate cancer metastatic:0.22), (ADO-TRASTUZUMAB EMTANSINE:Prostate cancer metastatic:1.02), (AGALSIDASE BETA:Prostate cancer metastatic:0.3), (ALCOHOL:Prostate cancer metastatic:0.31), (ALISKIREN HEMIFUMARATE:Prostate cancer metastatic:0.21), (AMANTADINE HYDROCHLORIDE:Prostate cancer metastatic:0.09), (AMILORIDE HYDROCHLORIDEHYDROCHLOROTHIAZIDE:Prostate cancer metastatic:0.31), (AMISULPRIDE:Prostate cancer metastatic:0.2), (AMLODIPINE BESYLATEHYDROCHLOROTHIAZIDEVALSARTAN:Prostate cancer metastatic:0.2), (AMLODIPINEPERINDOPRIL:Prostate cancer metastatic:0.04), (AMLODIPINEVALSARTAN:Prostate cancer metastatic:0.3), (AMPHETAMINE ASPARTATEAMPHETAMINE SULFATEDEXTROAMPHETAMINE SACCHARATEDEXTROAMPHETAMINE SULFATE:Prostate cancer metastatic:0.94), (AMPICILLIN:Prostate cancer metastatic:0.11), (ANASTROZOLE:Prostate cancer metastatic:2.15), (ARMODAFINIL:Prostate cancer metastatic:0.69), (AXITINIB:Prostate cancer metastatic:0.44), (BORTEZOMIB:Prostate cancer metastatic:0.52), (BUDESONIDEFORMOTEROL FUMARATE:Prostate cancer metastatic:0.28), (BUSPIRONE HYDROCHLORIDE:Prostate cancer metastatic:0.91), (CALCITONIN:Prostate cancer metastatic:0.27), (CALCITONIN SALMON:Prostate cancer metastatic:0.4), (CARISOPRODOL:Prostate cancer metastatic:0.28), (CARVEDILOL PHOSPHATE:Prostate cancer metastatic:1.67), (CEFAZOLIN SODIUM:Prostate cancer metastatic:0), (CERTOLIZUMAB PEGOL:Prostate cancer metastatic:0.68), (CHLORDIAZEPOXIDE HYDROCHLORIDECLIDINIUM BROMIDE:Prostate cancer metastatic:0.13), (CILASTATIN SODIUMIMIPENEM:Prostate cancer metastatic:0.23), (CLOSTRIDIUM BUTYRICUM SPORES STRAIN M-55:Prostate cancer metastatic:0.04), (CLOZAPINE:Prostate cancer metastatic:0.46), (DACLIZUMAB:Prostate cancer metastatic:0), (DANAZOL:Prostate cancer metastatic:0.41), (DAPAGLIFLOZIN PROPANEDIOL:Prostate cancer metastatic:0.09), (DEVICE:Prostate cancer metastatic:0.1), (DOFETILIDE:Prostate cancer metastatic:0.52), (DOLASETRON MESYLATE:Prostate cancer metastatic:0.2), (DOXEPIN HYDROCHLORIDE:Prostate cancer metastatic:0.17), (ECONAZOLE:Prostate cancer metastatic:0.47), (EDOXABAN TOSYLATE:Prostate cancer metastatic:0.93), (ELETRIPTAN HYDROBROMIDE:Prostate cancer metastatic:0.6), (EPIRUBICIN HYDROCHLORIDE:Prostate cancer metastatic:0.11), (ERIBULIN:Prostate cancer metastatic:0.01), (ERYTHROMYCIN:Prostate cancer metastatic:0.45), (ESTROGENS, CONJUGATEDMEDROXYPROGESTERONE ACETATE:Prostate cancer metastatic:0.33), (EVOLOCUMAB:Prostate cancer metastatic:0.39), (EXEMESTANE:Prostate cancer metastatic:0.97), (FENOFIBRIC ACID:Prostate cancer metastatic:0.04), (FENOTEROL HYDROBROMIDEIPRATROPIUM BROMIDE:Prostate cancer metastatic:0.12), (FEXOFENADINEPSEUDOEPHEDRINE:Prostate cancer metastatic:0.17), (FONDAPARINUX SODIUM:Prostate cancer metastatic:0.23), (FORMOTEROL FUMARATE:Prostate cancer metastatic:0.52), (FORMOTEROL FUMARATE DIHYDRATE\MOMETASONE FUROATE:Prostate cancer metastatic:0.58), (FULVESTRANT:Prostate cancer metastatic:0.32), (FUSIDIC ACID:Prostate cancer metastatic:0.23), (GEFITINIB:Prostate cancer metastatic:0.08), (GEMCITABINEGEMCITABINE HYDROCHLORIDE:Prostate cancer metastatic:0.2), (GIMERACILOTERACILTEGAFUR:Prostate cancer metastatic:0.51), (HEPARIN CALCIUM:Prostate cancer metastatic:0.5), (HERBALSTURMERIC:Prostate cancer metastatic:0.38), (HYDROXYCHLOROQUINE DIPHOSPHATE:Prostate cancer metastatic:0.05), (HYDROXYUREA:Prostate cancer metastatic:0.4), (HYDROXYZINE PAMOATE:Prostate cancer metastatic:1.22), (IBRUTINIB:Prostate cancer metastatic:0.64), (ILOPROST:Prostate cancer metastatic:0.84), (LAPATINIB DITOSYLATE:Prostate cancer metastatic:0.31), (LETROZOLE:Prostate cancer metastatic:1.11), (LEVALBUTEROL HYDROCHLORIDE:Prostate cancer metastatic:1.19), (LEVOLEUCOVORIN CALCIUM:Prostate cancer metastatic:0.17), (LIOTHYRONINE SODIUM:Prostate cancer metastatic:0.01), (LISDEXAMFETAMINE DIMESYLATE:Prostate cancer metastatic:0.23), (LITHIUM CARBONATE:Prostate cancer metastatic:0.94), (METAXALONE:Prostate cancer metastatic:0.81), (METFORMIN HYDROCHLORIDEVILDAGLIPTIN:Prostate cancer metastatic:0.38), (METHENAMINE HIPPURATE:Prostate cancer metastatic:0.34), (METHIMAZOLE:Prostate cancer metastatic:0.97), (METHOCARBAMOL:Prostate cancer metastatic:0.7), (METHYLPREDNISOLONE ACETATE:Prostate cancer metastatic:0.39), (MOXIFLOXACIN:Prostate cancer metastatic:0.07), (MYCOPHENOLATE MOFETILMYCOPHENOLATE MOFETIL HYDROCHLORIDE:Prostate cancer metastatic:0.2), (NADOLOL:Prostate cancer metastatic:0.28), (NICARDIPINE:Prostate cancer metastatic:0.01), (NILOTINIB:Prostate cancer metastatic:1.53), (NINTEDANIB:Prostate cancer metastatic:0.47), (NORTRIPTYLINE HYDROCHLORIDE:Prostate cancer metastatic:0.01), (OLODATEROL HYDROCHLORIDETIOTROPIUM BROMIDE MONOHYDRATE:Prostate cancer metastatic:0.67), (OXCARBAZEPINE:Prostate cancer metastatic:1.07), (PANITUMUMAB:Prostate cancer metastatic:0.02), (PEMETREXED:Prostate cancer metastatic:0.37), (PEMETREXED DISODIUM:Prostate cancer metastatic:0.77), (PENTOSAN POLYSULFATE SODIUM:Prostate cancer metastatic:0.09), (PERTUZUMAB:Prostate cancer metastatic:0.35), (PIROXICAM:Prostate cancer metastatic:0.06), (PONATINIB:Prostate cancer metastatic:0.41), (PRAMIPEXOLE DIHYDROCHLORIDE:Prostate cancer metastatic:1.15), (PRIMIDONE:Prostate cancer metastatic:1.22), (PROGESTERONE:Prostate cancer metastatic:0.48), (RALOXIFENE HYDROCHLORIDE:Prostate cancer metastatic:0.23), (RAMUCIRUMAB:Prostate cancer metastatic:0.19), (REGORAFENIB:Prostate cancer metastatic:0.5), (RIVASTIGMINE:Prostate cancer metastatic:0.44), (RIZATRIPTAN BENZOATE:Prostate cancer metastatic:0.24), (ROFLUMILAST:Prostate cancer metastatic:0.76), (SACUBITRILVALSARTAN:Prostate cancer metastatic:0.08), (SEVELAMER HYDROCHLORIDE:Prostate cancer metastatic:0.15), (SODIUM OXYBATE:Prostate cancer metastatic:0.89), (SULFAMETHOXAZOLE:Prostate cancer metastatic:0.09), (SUMATRIPTAN:Prostate cancer metastatic:0.02), (SUMATRIPTANSUMATRIPTAN SUCCINATE:Prostate cancer metastatic:0.5), (SUNITINIB MALATE:Prostate cancer metastatic:0.5), (TERBINAFINE HYDROCHLORIDE:Prostate cancer metastatic:0.49), (TESTOSTERONE CYPIONATE:Prostate cancer metastatic:0.18), (TETRACYCLINE:Prostate cancer metastatic:0.23), (THEOPHYLLINE ANHYDROUS:Prostate cancer metastatic:0.15), (THYROID, PORCINE:Prostate cancer metastatic:0.2), (TICAGRELOR:Prostate cancer metastatic:0.05), (VITAMIN A:Prostate cancer metastatic:0.17), (DILTIAZEM:Prostate infection:0.07), (DILTIAZEM HYDROCHLORIDE:Prostate infection:0.16), (EVOLOCUMAB:Prostate infection:0.11), (CANDESARTAN:Prostatic adenoma:0.09), (SACUBITRILVALSARTAN:Prostatic adenoma:0), (.ALPHA.1-PROTEINASE INHIBITOR HUMAN:Prostatic disorder:0.12), (ACETAMINOPHENOXYCODONE HYDROCHLORIDEOXYCODONE TEREPHTHALATE:Prostatic disorder:0.35), (ACETAZOLAMIDE:Prostatic disorder:0.94), (ALBIGLUTIDE:Prostatic disorder:0.44), (ALCOHOL:Prostatic disorder:0.34), (ALEMTUZUMAB:Prostatic disorder:0.04), (ALENDRONIC ACID:Prostatic disorder:0.25), (ALFACALCIDOL:Prostatic disorder:0.44), (AMISULPRIDE:Prostatic disorder:0.34), (AMLODIPINE BESYLATEATORVASTATIN CALCIUM:Prostatic disorder:0.18), (ARIPIPRAZOLE LAUROXIL:Prostatic disorder:0.25), (BUPRENORPHINE HYDROCHLORIDE:Prostatic disorder:0.3), (CALCIUM\MAGNESIUM:Prostatic disorder:0.03), (CALCIUM\MAGNESIUM\ZINC:Prostatic disorder:0.42), (CARVEDILOL PHOSPHATE:Prostatic disorder:1.58), (CEFTRIAXONE SODIUM:Prostatic disorder:0.05), (CHLORDIAZEPOXIDE HYDROCHLORIDECLIDINIUM BROMIDE:Prostatic disorder:0.25), (CICLESONIDE:Prostatic disorder:0.63), (CLINDAMYCIN:Prostatic disorder:0.34), (CORTICOTROPIN:Prostatic disorder:0.53), (DABIGATRAN:Prostatic disorder:0.59), (DACLIZUMAB:Prostatic disorder:1.05), (DALTEPARIN:Prostatic disorder:0), (DANAZOL:Prostatic disorder:0.26), (DESLORATADINE:Prostatic disorder:1.05), (DEXTROAMPHETAMINE SULFATE:Prostatic disorder:0.22), (DIPHENHYDRAMINE:Prostatic disorder:0.95), (DOXEPIN HYDROCHLORIDE:Prostatic disorder:0.44), (ECONAZOLE:Prostatic disorder:0.13), (ELETRIPTAN HYDROBROMIDE:Prostatic disorder:0.73), (EMPAGLIFLOZINLINAGLIPTIN:Prostatic disorder:0.94), (EPINEPHRINE:Prostatic disorder:0.83), (EPLERENONE:Prostatic disorder:0.91), (ERYTHROMYCIN:Prostatic disorder:0.12), (ESTRADIOL:Prostatic disorder:0.07), (ESTROGENS, CONJUGATED:Prostatic disorder:0.82), (ESTROGENS, CONJUGATEDMEDROXYPROGESTERONE ACETATE:Prostatic disorder:0), (FENOFIBRIC ACID:Prostatic disorder:0.2), (FENOTEROL HYDROBROMIDEIPRATROPIUM BROMIDE:Prostatic disorder:0.02), (FEXOFENADINEPSEUDOEPHEDRINE:Prostatic disorder:0.33), (FILGRASTIM:Prostatic disorder:0.11), (FLUVOXAMINE MALEATE:Prostatic disorder:0.22), (FORMOTEROL FUMARATE\MOMETASONE FUROATE:Prostatic disorder:0.3), (FUSIDIC ACID:Prostatic disorder:0.07), (GUANFACINE:Prostatic disorder:0.25), (GUANFACINE HYDROCHLORIDE:Prostatic disorder:0.02), (HYDROCHLOROTHIAZIDEIRBESARTAN:Prostatic disorder:1.02), (HYDROCHLOROTHIAZIDETELMISARTAN:Prostatic disorder:0.63), (HYDROCODONE BITARTRATEIBUPROFEN:Prostatic disorder:0.01), (HYDROXYCHLOROQUINE SULFATE:Prostatic disorder:0.51), (HYDROXYUREA:Prostatic disorder:0.47), (ILOPROST:Prostatic disorder:0.57), (IMIPRAMINE HYDROCHLORIDE:Prostatic disorder:0.19), (INDAPAMIDEPERINDOPRIL:Prostatic disorder:1.05), (INTERFERON ALFA-2A:Prostatic disorder:0.3), (IRINOTECAN HYDROCHLORIDE:Prostatic disorder:0.1), (LABETALOLLABETALOL HYDROCHLORIDE:Prostatic disorder:0.86), (LIDOCAINE HYDROCHLORIDE:Prostatic disorder:0.74), (LOMITAPIDE MESYLATE:Prostatic disorder:0.71), (MEPOLIZUMAB:Prostatic disorder:0.42), (METFORMIN HYDROCHLORIDEROSIGLITAZONE MALEATE:Prostatic disorder:0.12), (METHENAMINE HIPPURATE:Prostatic disorder:0.63), (METHYLPREDNISOLONE ACETATE:Prostatic disorder:0.43), (MIDODRINE HYDROCHLORIDE:Prostatic disorder:0.06), (MOXIFLOXACIN:Prostatic disorder:0.02), (NIRAPARIB:Prostatic disorder:0.13), (NORTRIPTYLINE HYDROCHLORIDE:Prostatic disorder:0.19), (OXYBUTYNINOXYBUTYNIN CHLORIDE:Prostatic disorder:0.19), (PANCRELIPASE:Prostatic disorder:0.01), (PANTOPRAZOLE MAGNESIUM:Prostatic disorder:1.59), (PATIROMER:Prostatic disorder:0.49), (PEGINTERFERON BETA-1A:Prostatic disorder:0.78), (PEMETREXED DISODIUM:Prostatic disorder:0.32), (PENTOXIFYLLINE:Prostatic disorder:0.76), (PHYTONADIONE:Prostatic disorder:0.02), (PILOCARPINE:Prostatic disorder:0.02), (PIROXICAM:Prostatic disorder:0.22), (PREDNISOLONEPREDNISOLONE ACETATEPREDNISOLONE SODIUM SUCCINATE:Prostatic disorder:0.02), (RABEPRAZOLE:Prostatic disorder:0.92), (RALOXIFENE HYDROCHLORIDE:Prostatic disorder:0.67), (REGORAFENIB:Prostatic disorder:0.16), (RISEDRONATE SODIUM:Prostatic disorder:0.44), (RIZATRIPTAN BENZOATE:Prostatic disorder:0.47), (SELEGILINE:Prostatic disorder:0.25), (SEVELAMER CARBONATE:Prostatic disorder:0.05), (SUMATRIPTAN:Prostatic disorder:0.61), (SUMATRIPTAN SUCCINATE:Prostatic disorder:0.19), (SUMATRIPTANSUMATRIPTAN SUCCINATE:Prostatic disorder:0.8), (TERBINAFINE HYDROCHLORIDE:Prostatic disorder:0.53), (TETRACYCLINE:Prostatic disorder:0.62), (TINZAPARIN:Prostatic disorder:0.02), (VITAMIN A:Prostatic disorder:0.4), (VORTIOXETINE HYDROBROMIDE:Prostatic disorder:0.01), (ZAFIRLUKAST:Prostatic disorder:0.18), (ZILEUTON:Prostatic disorder:0.04), (ZOLMITRIPTAN:Prostatic disorder:0.02), (ANASTROZOLE:Prostatitis:0.19), (BISACODYL OR DOCUSATE SODIUM:Prostatitis:0.06), (BUPROPION:Prostatitis:0.04), (CALCIUM ACETATE:Prostatitis:0.02), (CALCIUM CARBONATECHOLECALCIFEROL:Prostatitis:0.8), (CANDESARTAN:Prostatitis:0.61), (CE RTOLIZUMAB PEGOL:Prostatitis:0.23), (CHOLESTYRAMINE:Prostatitis:0.04), (CLONIDINE:Prostatitis:0.2), (CLOTRIMAZOLE:Prostatitis:0.03), (DASATINIB:Prostatitis:0.5), (DIPHENHYDRAMINE:Pro statitis:0.51), (DULOXETINE:Prostatitis:0.3), (EPLERENONE:Prostatitis:0.14), (ERYTHROPOI ETIN:Prostatitis:0.11), (ESZOPICLONE:Prostatitis:0.28), (EVOLOCUMAB:Prostatitis:0.69), (FE RROUS SULFATEFERROUS SULFATE, DRIED:Prostatitis:1.01), (FLECAINIDE:Prostatitis:0.38), (FLUTICASONE:Prostatitis:0.07), (GL IPIZIDE:Prostatitis:0.39), (HEPARIN SODIUM:Prostatitis:0.44), (HYDROCODONE:Prostatitis:0.15), (IBRUTINIB:Prostatitis:0.89), (I NSULIN HUMAN:Prostatitis:0.22), (ISOSORBIDE MONONITRATE:Prostatitis:0.48), (LEDIPASVIRSOFOSBUVIR:Prostatitis:0.66), (LERCANI DIPINE HYDROCHLORIDE:Prostatitis:0.32), (LOVASTATIN:Prostatitis:0.82), (METAXALONE:Prost atitis:0.19), (OXCARBAZEPINE:Prostatitis:0.37), (PANTOPRAZOLE MAGNESIUM:Prostatitis:0.15), (PRAMIPEXOLE:Prostatitis:0.4), (RANOLAZINE:Prostatitis:0.56), (SACUBITRILVALSARTAN:Prostatitis:0.52), (SECUKINUMAB:Prostatitis:0.04), (SOTA LOL:Prostatitis:0.37), (TEMAZEPAM:Prostatitis:0.36), (TIMOLOL:Prostatitis:0.08), (TIOTROPI UM:Prostatitis:0.02), (TIZANIDINE HYDROCHLORIDE:Prostatitis:0.33), (TRIAMCINOLONE ACETONIDE:Prostatitis:0.54), (VANCOMYCIN HYDROCHLORIDE:Prostatitis:0.03), (VARENICLINE TARTRATE:Prostatitis:0.26), (ACENOCOUMAROL:Prostatomegaly:0.16), (ACETAMINOPHE NTRAMADOL:Prostatomegaly:0.09), (ALFACALCIDOL:Prostatomegaly:0.34), (ALUMINUM HYDROXIDEDIMETHICONEMAGNESIUM HYDROXIDE:Prostatomegaly:0.39), (ANASTROZOLE:Prostatomegaly:1.44), (APREPITANT: Prostatomegaly:0.06), (AZILSARTAN KAMEDOXOMIL:Prostatomegaly:0.07), (BETAMETHASONE DIPROPIONATECALCIPOTRIENECALCIPOTRIENE HYDRATE:Prostatomegaly:0.15), (CALCIPOTRIENE:Prostatomegaly:0.02), (CALCITONIN:Pr ostatomegaly:0.25), (CALCIUM CARBONATEVITAMIN D:Prostatomegaly:0.14), (CALCIUM\MAGNESIUM\ZINC:Prostatomegaly:0.36), (CANDESAR TAN CILEXETILHYDROCHLOROTHIAZIDE:Prostatomegaly:1.66), (CARVEDILOL PHOSPHATE:Prostatomegaly:1.8), (CLINDAMYCIN:Prostatomegaly:0.79), (CODEINE:Prostat omegaly:0.37), (DABIGATRAN:Prostatomegaly:0.29), (DACLIZUMAB:Prostatomegaly:0.83), (DARBEPOETIN ALFA:Prostatomegaly:1.18), (DASATINIB:Prostatomegaly:1.8), (DEFLAZACORT:Prostatomeg aly:0.43), (DIMENHYDRINATE:Prostatomegaly:0.33), (DORZOLAMIDE HYDROCHLORIDE:Prostatomegaly:0.18), (DOXYCYCLINE HYCLATE:Prostatomegaly:0.08), (ECONAZOLE:Prostatomegaly:0.07), (EDOXABAN TOSYLATE:Prostatomegaly:0.76), (ELETRIPTAN HYDROBROMIDE:Prostatomegaly:1.09), (ESTRADIOL:Prostatomegaly:0.03), (ESTROGENS, CONJUGATED:Prostatomegaly:0.84), (ESTROGENS, CONJUGATEDMEDROXYPROGESTERONE ACETATE:Prostatomegaly:0.19), (ETORICOXIB:Prostatomegaly:0.33), (EVOLOCUMAB:Prost atomegaly:2.18), (FLUDROCORTISONE ACETATE:Prostatomegaly:0.64), (FLUOCINOLONE ACETONIDE:Prostatomegaly:0.1), (FLUTICASONE FUROATE:Prostatomegaly:0.76), (FLUTICASONE FUROATESALMETEROL XINAFOATE:Prostatomegaly:0.92), (FORMOTEROL FUMARATE\MOMETASONE FUROATE:Prostatomegaly:0.68), (GLYCOPYRROLATEINDACATEROL MALEATE:Prostatomegaly:0.02), (GLYCOPYRRONIUMINDACATEROL:Prostatomegaly:0.96), (HYDROCODONE BITARTRATEIBUPROFEN:Prostatomegaly:0.3), (ILOPROST:Prostatomegaly:0.57), (INDAC ATEROL:Prostatomegaly:0.38), (INDAPAMIDEPERINDOPRIL:Prostatomegaly:0.69), (INTER FERON ALFA-2A:Prostatomegaly:0.05), (IODINE:Prostatomegaly:0.21), (KETOROLACKETOROLAC TROMETHAMINE:Prostatomegaly:0.14), (LABETALOL HYDROCHLORIDE:Prostatomegaly:0.01), (LEDIPASVIRSOFOSBUVIR:Prostatomegaly:0.62), (LERCANIDIPINE:Prostatomegaly:1.05), (LERCANIDIPINE HYDROCHLORIDE:Prostatomegaly:0.67), (LEVOMEFOLATE CALCIUM:Prostatomegaly:0.18), (LISDEXAMFETAMINE DIMESYLATE:Prostatomegaly:0.84), (LOMITAPIDE MESYLATE:Prostatomegaly:0.83), (MEPOLIZUMAB:Prostatomegaly:0.54), (METFORMIN HYDROCHLORIDEVILDAGLIPTIN:Prostatomegaly:0.18), (METHENAMINE HIPPURATE:Prostatomegaly:1.18), (METHYLPHENIDATE HYDROCHLORIDE:Prostatomegaly:0.77), (MINOCYCLINE HYDROCHLORIDE:Prostatomegaly:0.04), (MORPHINE SULFATE\NALTREXONE HYDROCHLORIDE:Prostatomegaly:0.12), (MOXIFLOXACIN:Prostatomegaly:0.21), (MUPIR OCINMUPIROCIN CALCIUM:Prostatomegaly:0.38), (NALOXONE HYDROCHLORIDEOXYCODONE HYDROCHLORIDE:Prostatomegaly:1.19), (NALTREXONE:Prostatomegaly:0.26), (NILOTINI B:Prostatomegaly:1.13), (NIRAPARIB:Prostatomegaly:0.28), (OLMESARTAN:Prostatomegaly: 0.17), (OXYBUTYNINOXYBUTYNIN CHLORIDE:Prostatomegaly:0.2), (PANTOPRAZOLE MAGNESIUM:Prostatomegaly:1.93), (PEGINTERFERON BETA-1A:Prostatomegaly:0.74), (PIROXICAM:Prostatomegaly:0.34), (PONATINIB:Prostatomegaly:1.39), (PRAZOSIN HYDROCHLORIDE:Prostatomegaly:0.14), (PREDNISOLONE ACETATE:Prostatomegaly:1.11), (PROPAFENONE HYDROCHLORIDE:Prostatomegaly:0.52), (PSYLLIUM HUSK:Prostatomegaly:0.59), (PYRAZINAMIDE:Prostatomegaly:0.71), (RALOXIFENE HYDROCHLORIDE:Prostatomegaly:0.96), (REGORAFENIB:Prostatomegaly:0.48), (RIZATRI PTAN:Prostatomegaly:0.01), (SECUKINUMAB:Prostatomegaly:0.93), (SELEGILINE:Prostatom egaly:0.36), (SITAGLIPTIN:Prostatomegaly:0.43), (SODIUM PHOSPHATE, DIBASIC\SODIUM PHOSPHATE, MONOBASIC:Prostatomegaly:0.06), (SULFAMETHOXAZOLE:Prostatomegaly:0.38), (SUMA TRIPTAN:Prostatomegaly:0.74), (SUMATRIPTANSUMATRIPTAN SUCCINATE:Prostatomegaly:0.8), (TETRACYCLINE:Prostatomegaly:1.31), (TOLTERODINE: Prostatomegaly:0.24), (UMECLIDINIUM BROMIDE:Prostatomegaly:0.29), (VALGANCICLOVIR HYDROCHLORIDE:Prostatomegaly:0.39), (VILAZODONE HYDROCHLORIDE:Prostatomegaly:0.64), (ZILEUTON:Prostatomegaly:0.11), (ZONISAMIDE: Prostatomegaly:0.06), (ALBUTEROL SULFATEIPRATROPIUM BROMIDE:Proteinuria:0), (AMIODARONE:Proteinuria:0.71), (ANASTROZOLE:Proteinuria:0.04), (BISOPROLOL FUMARATE:Proteinuria:0.64), (CELECOXIB:Proteinuria:0.68), (CERTOLIZUMAB PEGOL:Proteinuria:0.84), (CLINDAMYCINCLINDAMYCIN PHOSPHATE:Proteinuria:0.23), (CYCLOBENZAPRINE:Proteinuria:0.08), (DEVICE:Proteinuri a:0.49), (DICLOFENAC:Proteinuria:0), (DIVALPROEX SODIUM:Proteinuria:0.48), (EPINEPHRINE:Proteinuria:0.32), (ESZOPICLONE:Proteinuria:0.08), (EVOLOCUMAB:Proteinuria:0.82), (HYDROCHLOROTHIAZIDELISINOPRIL:Proteinuria: 0.09), (ISOSORBIDE:Proteinuria:0.18), (LEDIPASVIRSOFOSBUVIR:Proteinuria:0.2), (LERC ANIDIPINE:Proteinuria:0.29), (LOVASTATIN:Proteinuria:0.62), (NAPROXEN SODIUM:Proteinuria:0.02), (OXCARBAZEPINE:Proteinuria:0.07), (POLYETHYLENE GLYCOLS:Proteinuria:0.17), (PRAVASTATIN:Proteinuria:0.77), (RABEPRAZOLE SODIUM:Proteinuria:0.13), (SACUBITRILVALSARTAN:Proteinuria:0.24), (SECUKINUMAB: Proteinuria:0.15), (TAMSULOSIN:Proteinuria:0.73), (TERIFLUNOMIDE:Proteinuria:0.22), (TI OTROPIUM BROMIDE MONOHYDRATE:Proteinuria:0.5), (ABOBOTULINUMTOXINA:Pruritus:0.76), (ACETAMIN OPHEN\CAFFEINEOPIUM:Pruritus:0.37), (ADENINE:Pruritus:0.55), (AJMALINE:Pruritus:0.12), (ALUMINUM HYDROXIDEMAGNESIUM OXIDEOXETHAZAINE:Pruritus:0.04), (ALUMINUM SILICATE:Pruritus:0.15), (AMIKACIN:Pruritus:1.17), (AMIKACIN SULFATE:Pruritus:0.89), (AMINO ACIDSDEXTROSEELECTROLYTES NOS:Pruritus:2.07), (AMINOSALICYLIC ACID:Pruritus:0.29), (AMISULPRIDE:Pruritus:1.32), (AMLODIPINE BESYLATEATORVASTATIN CALCIUM:Pruritus:1.38), (AMMONIUM CHLORIDEDEXTROMETHORPHAN\SODIUM CITRATE:Pruritus:0.61), (AMOXAPINE:Pruritus:0.88), (AMOXICILLINCLARITHROMYCI NESOMEPRAZOLE:Pruritus:0.99), (AMPICILLIN SODIUM:Pruritus:0.05), (ANAGLIPTIN:Pruritus:0.21), (ANAGRELIDE:Pruritus:0.23), (ARIPIP RAZOLE LAUROXIL:Pruritus:0.25), (ASCORBIC ACIDBIOTINCYANOCOBALAMINFOLIC ACIDNIACINPANTOTHENIC ACIDPYRIDOXINE\RIBOFLAVINTHIAMINE:Pruritus:0.33), (ASPIRIN\CAFFEINE:Pruritu s:0.02), (ASPIRINDIPYRIDAMOLE:Pruritus:0.24), (ASPIRINMAGNESIUM OXIDE:Pruritus:0.94), (ATRACURIUM:Pruritus:0.15), (ATRACURIUM BESYLATE:Pruritus:0.05), (BACITRACIN ZINC:Pruritus:0.13), (BELATACEPT:Pruritus:0.52), (BENZETHONIUM CHLORIDE:Pruritus:0.01), (BENZOCAINE:Pruritus:0.45), (BEPRIDIL:Pruritus:1.4), (BERAPR OST:Pruritus:1.14), (BETAMETHASONE DIPROPIONATECALCIPOTRIENE HYDRATE:Pruritus:0.36), (BETAMETHASONE SODIUM PHOSPHATENEOMYCIN SULFATE:Pruritus:0.04), (LACTIS:Pruritus:0.18), (BOTULINUM TOXIN TYPE A:Pruritus:0.41), (BRIVARACETAM:Pruritus:0.36), (BUDESONIDEFORMOTEROL FUMARATE:Pruritus:1.6), (BUPIVACAINE HYDROCHLORIDE:Pruritus:0.45), (BUPRENORPHINENALOXONE:Pruritus:0.14), (BUTAL BITAL:Pruritus:0.43), (BUTYLSCOPOLAMINE:Pruritus:0.01), (CABAZITAXEL:Pruritus:0.44), (CABOZANTINIB:Pruritus:0.45), (CALCIUM PANTOTHENATECYANOCOBALAMINNIACINAMIDEPYRIDOXINE HYDROCHLORIDE\RIBOFLAVINTHIAMINE MONONITRATE:Pruritus:0.48), (CALCIUM\MAGNESIUM:Pruritus:1.77), (CALCIUMVITA MINS:Pruritus:0.06), (CARBIDOPA:Pruritus:0.77), (CARBOHYDRATESFATTY ACIDSMINERALSPROTEINVITAMINS:Pruritus:0.1), (CARMUSTINE:Pruritus:1.04), (CEF PROZIL:Pruritus:0.6), (CEFTAZIDIME SODIUM:Pruritus:0.04), (CELIPROLOL:Pruritus:0.47), (CEPHARANTHINE:Pruritus:0.35), (CE RTOPARIN SODIUM:Pruritus:0.12), (CHLORAMBUCIL:Pruritus:1.38), (CHLORPHENIRAMINEHYDRO CODONE:Pruritus:1.42), (CHOLESTEROL:Pruritus:0.22), (CHONDROITIN SULFATE (CHICKEN):Pruritus:0.39), (CICLOPIROX OLAMINE:Pruritus:0.14), (CIFENLINE:Pruritus:0.41), (CIPROFIBRATE:Pruritus:0.87), (CLIKS TAR:Pruritus:0.09), (CLOFAZIMINE:Pruritus:0.07), (CLOMIPRAMINE HYDROCHLORIDE:Pruritus:1.27), (CLOPERASTINE:Pruritus:0.09), (CLOZAPINE:Pruritus:1.85), (COBICISTATELVITEGRAVIREMTRICITABINETENOFOVIR ALAFENAMIDE FUMARATE:Pruritus:0.57), (COBICISTATELVITEGRAVIREMTRICITABINETENOFOVI R DISOPROXIL FUMARATE:Pruritus:0.39), (CORTISONE ACETATE:Pruritus:0.13), (CYCLOPENTOLATE:Pruritus:0.19), (DACLIZUMAB:Pruritus:2.52), (DANTROLENE SODIUM:Pruritus:0.77), (DAPAGLIFLOZIN PROPANEDIOLMETFORMIN HYDROCHLORIDE:Pruritus:0.29), (DEGARELIX:Pruritus:0.65), (DEXTROAMPHETAMINE SULFATE:Pruritus:0.99), (DIAZOXIDE:Pruritus:0.15), (DICLOFENAC EPOLAMINE:Pruritus:0.03), (DILAZEP:Pruritus:1.42), (DIMEMORFAN PHOSPHATE:Pruritus:0.1), (DIPHENIDOL HYDROCHLORIDE:Pruritus:0.83), (DISOPYRAMIDE:Pruritus:0.21), (DOBUTAMINE HYDROCHLORIDE:Pruritus:1.18), (DOLASETRON MESYLATE:Pruritus:1.08), (DOLUTEGRAVIR:Pruritus:0.03), (DORIPENEM:Pruritus:0.12), (D ORZOLAMIDE HYDROCHLORIDE:Pruritus:0.92), (DOXYLAMINE SUCCINATEPYRIDOXINE HYDROCHLORIDE:Pruritus:0.39), (DROPERIDOL:Pruritus:0.02), (ECABET:Pruritus:0.14), (E CABET SODIUM:Pruritus:0.01), (EDARAVONE:Pruritus:0.82), (ELOTUZUMAB:Pruritus:1.35), (EMP AGLIFLOZINLINAGLIPTIN:Pruritus:1.23), (ENALAPRIL MALEATEHYDROCHLOROTHIAZIDE:Pruritus:0.49), (ENALAPRIL MALEATELERCANIDIPINE HYDROCHLORIDE:Pruritus:0.74), (ENFUVIRTIDE:Pruritus:0.06), (EPHEDRINE HYDROCHLORIDEGUAIFENESIN:Pruritus:0.03), (EPIRUBICIN HYDROCHLORIDE:Pruritus:0.17), (ERIBULIN:Pruritus:0.75), (ESOMEPRAZOLE SODIUM:Pruritus:0.41), (ESTRADIOLLEVONORGESTREL:Pruritus:0.03), (ESTRADIOLN ORETHINDRONE ACETATE:Pruritus:0.75), (ETHACRYNIC ACID:Pruritus:0.41), (ETHINYL ESTRADIOLNORGESTREL:Pruritus:0.58), (ETHOSUXIMIDE:Pruritus:0.03), (EUCALYPTO LMENTHOLMETHYL SALICYLATETHYMOL:Pruritus:0.23), (FAROPENEM SODIUM:Pruritus:0.49), (FENOTEROL HYDROBROMIDEIPRATROPIUM BROMIDE:Pruritus:0.95), (FERROUS BISGLYCINATE:Pruritus:0.11), (FERROUS SULFATEFOLIC ACID:Pruritus:0.67), (FISH OILTOCOPHEROL:Pruritus:0.09), (FLUNARIZINE HYDROCHLORIDE:Pruritus:0.11), (FLUOXETINE HYDROCHLORIDEOLANZAPINE:Pruritus:0.03), (FLUTAMIDE:Pruritus:0.31), (FLUVOXA MINE:Pruritus:0.1), (FONDAPARINUX:Pruritus:0.43), (FOSFLUCONAZOLE:Pruritus:0.07), (F OSFOMYCIN:Pruritus:0.7), (FOSFOMYCIN CALCIUM:Pruritus:0.23), (FOSPHENYTOINFOSPHENYTOIN SODIUM:Pruritus:1), (FROVATRIPTAN SUCCINATE:Pruritus:0.45), (FURSULTIAMINE:Pruritus:0.31), (GLIPIZIDEMETFORMIN HYDROCHLORIDE:Pruritus:0.4), (GLYBURIDEMETFORMIN HYDROCHLORIDE:Pruritus:0.46), (GLYCOPYRROLATEINDACATEROL MALEATE:Pruritus:0.67), (GUANABENZ ACETATE:Pruritus:0.53), (HYDROCHLOROTHIAZIDEQUINAPRIL HYDROCHLORIDE:Pruritus:0.65), (HYDROCODONE BITARTRATEIBUPROFEN:Pruritus:1.15), (HYDROQUINONE:Pruritus:0.24), (HYDROXYC HLOROQUINE DIPHOSPHATE:Pruritus:0.95), (HYOSCYAMINE SULFATEMETHENAMINEMETHYLENE BLUEPHENYL SALICYLATE\SODIUM PHOSPHATE, MONOBASIC, MONOHYDRATE:Pruritus:0.13), (IDARUBICIN:Pruritus:1.25), (IDARUBICIN HYDROCHLORIDE:Pruritus:0.39), (IDARUCIZUMAB:Pruritus:0.34), (IFENPRODIL:Pruritus: 0.11), (IGURATIMOD:Pruritus:0.41), (ILOPROST:Pruritus:1.58), (IMIPRAMINE HYDROCHLORIDE:Pruritus:2.22), (IMMUNE GLOBULIN NOS:Pruritus:0.8), (INFLIXIMAB-DYYB:Pruritus:1.18), (IRON POLYMALTOSE:Pruritus:0.11), (IRONVITAMINS:Pruritus:0.1), (ISAVUCONAZONIUM SULFATE:Pruritus:0.02), (ISOFLURANE:Pruritus:0.62), (ISONIAZIDPYRAZINAMIDERIFA MPIN:Pruritus:0.04), (ITOPRIDE:Pruritus:0.09), (ACIDOPHILUSLACTOBACILLUS DELBRUECKII SUBSP. BULGARICUS:Pruritus:0.58), (LANREOTIDE:Pruritus:0.29), (LARONIDASE:Pruritus:0.61), (L ENVATINIB:Pruritus:0.86), (LEVOTHYROXINE SODIUMPOTASSIUM IODIDE:Pruritus:0.35), (LINSIDOMINE:Pruritus:0.06), (LOMITAPIDE MESYLATE:Pruritus:1.17), (LOMUSTINE:Pruritus:0.46), (LUMACAFTOR:Pruritus:0.11), (LU TETIUM LU-177:Pruritus:0.24), (LYSOZYME HYDROCHLORIDE:Pruritus:0.7), (MEPOLIZUMAB:Pruritus:1.55), (METFORMIN HYDROCHLORIDESAXAGLIPTIN:Pruritus:0.24), (METFORMIN PAMOATE:Pruritus:0.45), (METHENAMINE:Pruritus:0.17), (METHENAMINE HIPPURATE:Pruritus:2.11), (METHENOLONE ENANTHATE:Pruritus:1.77), (METILDIGOXIN:Pruritus:1.02), (MIGLUSTAT:Pruritus:0.81), (MITIGLINIDE CALCIUM HYDRATEVOGLIBOSE:Pruritus:0.18), (MOEXIPRIL HYDROCHLORIDE:Pruritus:1.13), (MORPHINE SULFATE\NALTREXONE HYDROCHLORIDE:Pruritus:1.58), (MOXONIDINE:Pruritus:1.38), (NADROPARIN CALCIUM:Pruritus:1.79), (NEFAZODONE HYDROCHLORIDE:Pruritus:0.04), (NEPAFENAC:Pruritus:0.34), (NIRAPARIB:Pruritus:1.8), (NOREPINEPHRINE:Pruritus:1.22), (NORETHINDRONE:Pruritus:1.39), (NUSINERSEN:Prurit us:0.33), (OPIPRAMOL:Pruritus:0.58), (ORLISTAT:Pruritus:0.03), (OXYBUTYNINOXYBUT YNIN CHLORIDE:Pruritus:0.56), (PANOBINOSTAT:Pruritus:0.02), (PASIREOTIDE:Pruritus:0.07), (P ATIROMER:Pruritus:1.21), (PEGINTERFERON ALFA-2A OR PEGINTERFERON ALFA-2B OR PEGINTERFERON BETA-1A OR PEGINTERFERON LAMBDA-1A:Pruritus:0.2), (PEGINTERFERON BETA-1A:Pruritus:1.92), (PENICILLIN V BENZATHINE:Pruritus:0.21), (PENTOSTATIN:Pruritus:1.25), (PHENOBARBITAL SODIUM:Pruritus:0.46), (PIRITRAMIDE:Pruritus:0.04), (PLERIXAFOR:Pruritus:0.19), (PODO FILOX:Pruritus:0.15), (PORFIMER SODIUM:Pruritus:0.4), (POTASSIUM CITRATETRISODIUM CITRATE DIHYDRATE:Pruritus:1.19), (POTASSIUM PHOSPHATE, MONOBASIC:Pruritus:0.04), (PYRIDOXAL:Pruritus:0.94), (PYRIDOXAL PHOSPHATE:Pruritus:0.57), (QUINAPRIL:Pruritus:1.05), (QUINAPRIL HYDROCHLORIDE:Pruritus:0.16), (QUINAPRILQUINAPRIL HYDROCHLORIDE:Pruritus:0.69), (QUINIDINE GLUCONATE:Pruritus:0.16), (RILMENIDINE PHOSPHATE:Pruritus:0.66), (ROFECOXIB:Pruritus:0.42), (ROPIVACAINE:Pruritus:0.19), (RO PIVACAINE HYDROCHLORIDE:Pruritus:0.16), (RUFINAMIDE:Pruritus:0.64), (SAPROPTERIN DIHYDROCHLORIDE:Pruritus:0.67), (SARPOGRELATE HYDROCHLORIDE:Pruritus:0.27), (SELEGILINE HYDROCHLORIDE:Pruritus:0.06), (SELEXIPAG:Pruritus:0.29), (SERRAPEPTASE:Pruritus:0.1), (SIBUTRAMINE HYDROCHLORIDE:Pruritus:0.1), (SITAFLOXACIN:Pruritus:0.04), (SODIUM CITRATE:Pruritus:0.66), (SODIUM GUALENATE MONOHYDRATE:Pruritus:0.18), (SUNITINIB:Pruritus:0.35), (SUPLATAST TOSILATE:Pruritus:0.71), (TAZAROTENE:Pruritus:0.12), (TEDUGLUTIDE:Pruritus:0.45), (TE DUGLUTIDE\WATER:Pruritus:1.57), (TEMOCAPRIL HYDROCHLORIDE:Pruritus:0.63), (TESTOSTERONE UNDECANOATE:Pruritus:1.47), (THEOPHYLLINE:Pruritus:0.8), (THIAMYLAL SODIUM:Pruritus:0.11), (THIOPENTAL SODIUM:Pruritus:0.1), (THROMBOMODULIN ALFA:Pruritus:0.41), (TIMOLOL MALEATETRAVOPROST:Pruritus:0.57), (TINZAPARIN:Pruritus:1.48), (TRANDOLAPRIL:P ruritus:0.97), (TRIPTORELIN PAMOATE:Pruritus:0.36), (TROSPIUM:Pruritus:0.13), (UROKINASE:Pruritus:0.24), (VALDEC OXIB:Pruritus:0.08), (ZALEPLON:Pruritus:0.16), (ZALTOPROFEN:Pruritus:0.22), (ZICONOTI DE ACETATE:Pruritus:0.51), (ZINC ACETATE:Pruritus:0.03), (ZIV-AFLIBERCEPT:Pruritus:0.2), (.ALPHA.1-PROTEINASE INHIBITOR HUMAN:Pseudomonas infection:0.95), (ALBUMIN HUMAN:Pseudomonas infection:0.16), (AMPICILLIN SODIUMSULBACTAM SODIUM:Pseudomonas infection:0.04), (BORTEZOMIB:Pseudomonas infection:0.06), (BUMETANIDE:Pseudomonas infection:0.81), (BUPROPION:Pseudomonas infection:0.09), (BUPROPION HYDROCHLORIDE:Pseudomonas infection:0.88), (CALCIUM ACETATE:Pseudomonas infection:0.15), (CANAKINUMAB:Pseudomonas infection:0.09), (CANDESARTAN CILEXETIL:Pseudomonas infection:0.31), (CANDESARTAN CILEXETILHYDROCHLOROTHIAZIDE:Pseudomonas infection:0.03), (CEFOXITINCEFOXITIN SODIUM:Pseudomonas infection:0.06), (CERTOLIZUMAB PEGOL:Pseudomonas infection:0.89), (CHOLESTYRAMINE:Pseudomonas infection:0.44), (COLCHICINE:Pseudomonas infection:0.28), (DASATINIB:Pseudomonas infection:0.18), (DICLOFENAC SODIUM:Pseudomonas infection:0.69), (ELETRIPTAN HYDROBROMIDE:Pseudomonas infection:0.05), (EZETIMIBE:Pseudomonas infection:0.18), (FENOTEROL HYDROBROMIDEIPRATROPIUM BROMIDE:Pseudomonas infection:0.15), (FORMOTEROL:Pseudomonas infection:0.41), (FORMOTEROL FUMARATE\MOMETASONE FUROATE:Pseudomonas infection:1.21), (GLIPIZIDE:Pseudomonas infection:0.11), (GLYCOPYRRONIUMINDACATEROL:Pseudomonas infection:0.49), (HYDROMORPHONE HYDROCHLORIDE:Pseudomonas infection:0.13), (HYDROXYCHLOROQUINE DIPHOSPHATE:Pseudomonas infection:0.03), (HYDROXYZINE PAMOATE:Pseudomonas infection:0.09), (ILOPROST:Pseudomonas infection:0.37), (INDAPAMIDEPERINDOPRIL:Pseudomonas infection:0.22), (INTERFERON GAMMA-1B:Pseudomonas infection:0.05), (ISOSORBIDE MONONITRATE:Pseudomonas infection:0.37), (LABETALOLLABETALOL HYDROCHLORIDE:Pseudomonas infection:0.52), (LEVALBUTEROL:Pseudomonas infection:0.3), (LOVASTATIN:Pseudomonas infection:0.45), (MEPOLIZUMAB:Pseudomonas infection:0.88), (METAXALONE:Pseudomonas infection:0.04), (METOPROLOL TARTRATE:Pseudomonas infection:0.43), (NALOXONE HYDROCHLORIDEOXYCODONE HYDROCHLORIDE:Pseudomonas infection:0.08), (NEBIVOLOL:Pseudomonas infection:0.5), (NIFEDIPINE:Pseudomonas infection:0.42), (NILOTINIB:Pseudomonas infection:0.29), (NITROFURANTOIN:Pseudomonas infection:0.09), (NITROGLYCERIN:Pseudomonas infection:0.05), (NOREPINEPHRINE:Pseudomonas infection:0.03), (OXCARBAZEPINE:Pseudomonas infection:0.89), (OXYCODONE HYDROCHLORIDE:Pseudomonas infection:0.06), (PANTOPRAZOLE MAGNESIUM:Pseudomonas infection:1.4), (PONATINIB:Pseudomonas infection:0.3), (PRAVASTATIN SODIUM:Pseudomonas infection:0.58), (PREDNISOLONE SODIUM PHOSPHATE:Pseudomonas infection:0.21), (PRIMIDONE:Pseudomonas infection:0.11), (TEMAZEPAM:Pseudomonas infection:0.33), (THEOPHYLLINE:Pseudomonas infection:0), (TOFACITINIB CITRATE:Pseudomonas infection:0.02), (UMECLIDINIUM BROMIDEVILANTEROL TRIFENATATE:Pseudomonas infection:0.42), (VARENICLINE TARTRATE:Pseudomonas infection:0.32), (VITAMIN B:Pseudomonas infection:0.12), (.ALPHA.-TOCOPHEROLRETINOL:Psoriasis:0.02), (.ALPHA.-TOCOPHEROLVITAMINS:Psoriasis:0.28), (ABOBOTULINUMTOXINA:Psoriasis:1.15), (AC ETAMINOPHENBUTALBITALCAFFEINECODEINE PHOSPHATE:Psoriasis:0.41), (ACETAMINOPHEN\CAFFEINE:Psoriasis:1.28), (ACETAMIN OPHENDICHLORALPHENAZONEISOMETHEPTENE:Psoriasis:1.24), (ACETYLCARNITI NE HYDROCHLORIDE:Psoriasis:0.92), (ADEMETIONINE SULFATE TOSILATE:Psoriasis:0.06), (ADENOSINE TRIPHOSPHATE:Psoriasis:0.32), (ADO-TRASTUZUMAB EMTANSINE:Psoriasis:1.04), (AFATINIB:Psoriasis:0.39), (AGALSIDASE BETA:Psoriasis:1.94), (ALBUMIN BOVINETANNIC ACID:Psoriasis:0.07), (ALCOHOLALUMINUM CHLORIDE:Psoriasis:0.18), (ALEMTUZUMAB:Psoriasis:0.87), (ALGLUCOSIDASE ALFA:Psoriasis:0.14), (ALLANTOINBENZOCAINECAMPHOR (NATURAL)DIMETHICONEMENTHOL:Psoriasis:0.42), (ALUMINUM HYDROXIDEMAGNESIUM HYDROXIDESIMETHICONE:Psoriasis:0.28), (AMBROXOLCLENBUTEROL:Psoriasis:0.35), (AMIKACIN:Psoriasis:0.4), (AMILORIDE HYDROCHLORIDEFUROSEMIDE:Psoriasis:0.57), (AMINO ACIDSCARBOHYDRATESMINERALSSOYBEAN OILVITAMINS:Psoriasis:0.46), (AMINO ACIDSDEXTROSEELECTROLYTES NOS:Psoriasis:1.33), (AMINOBENZOIC ACIDCALCIUM PANTOTHENATECYSTINEKERATINTHIAMINE MONONITRATE:Psoriasis:0.06), (AMLODIPINELOSARTAN:Psoriasis:0.03), (MONTANA:Psoriasis:0.11), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Psoriasis:0.58), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE HYDROCHLORIDE:Psoriasis:0.02), (ASCORBIC ACIDBIOTINCYANOCOBALAMINFOLIC ACIDNIACINPANTOTHENIC ACIDPYRIDOXINE\RIBOFLAVINTHIAMINE:Psoriasis:0.69), (ASCORBIC ACIDPOLYETHYLENE GLYCOL 3350POTASSIUM CHLORIDE\SODIUM ASCORBATE\SODIUM CHLORIDE\SODIUM SULFATE:Psoriasis:0.08), (ASPIRIN\CAFFEINESALICYLAMIDE:Psoriasis:0.07), (ASUNAP REVIR:Psoriasis:1.09), (ATOMOXETINE:Psoriasis:0.09), (ATOVAQUONE:Psoriasis:0.64), (AT ROPINE:Psoriasis:0.76), (AVANAFIL:Psoriasis:0.37), (BACITRACINNEOMYCIN SULFATEPOLYMYXIN B SULFATE:Psoriasis:0.27), (BAMIFYLLINE HYDROCHLORIDE:Psoriasis:1.1), (BENSERAZIDE HYDROCHLORIDELEVODOPA:Psoriasis:0.43), (BENZALKONIUM CHLORIDE:Psoriasis:0.44), (BENZOCAINERESORCINOL:Psoriasis:0.01), (BENZOYL PEROXIDEERYTHROMYCIN:Psoriasis:0.36), (BEPRIDIL:Psoriasis:0.52), (BETAMETHASO NE ACETATEBETAMETHASONE SODIUM PHOSPHATE:Psoriasis:0.24), (BETAMETHASONE DIPROPIONATECLOTRIMAZOLEGENTAMICIN SULFATE:Psoriasis:0.08), (BETHANECHOL CHLORIDE:Psoriasis:1.3), (BEXAROTENE:Psoriasis:0.81), (BIMATOPROSTTIMOLOL:Psori asis:0.51), (BISMUTH SUBCITRATE POTASSIUMMETRONIDAZOLETETRACYCLINE HYDROCHLORIDE:Psoriasis:0.41), (BLONANSERIN:Psoriasis:0.38), (BORIC ACID:Psoriasis:0.2), (BORIC ACIDSODIUM BORATE:Psoriasis:0.29), (BORON:Psoriasis:0.18), (BOSUTINIB:Psoriasis:0.06), (BOTULINU M TOXIN TYPE A:Psoriasis:1.36), (BRINZOLAMIDETIMOLOL MALEATE:Psoriasis:0.62), (BROMOPRIDE:Psoriasis:0.75), (BUPRENORPHINENALOXON E:Psoriasis:0.52), (CABOZANTINIB S-MALATE:Psoriasis:0.5), (CAFFEINEISOMETHEPTENEMETHIMAZOLE:Psoriasis:0.21), (C ALCIUM BICARBONATE:Psoriasis:0.24), (CALCIUM CARBONATEETIDRONATE DISODIUM:Psoriasis:0.76), (CALCIUM\MAGNESIUM\ZINC:Psoriasis:2.36), (CALCIUMVIT AMINS:Psoriasis:0.79), (CARFILZOMIB:Psoriasis:1.21), (CARTEOLOL HYDROCHLORIDE:Psoriasis:0.58), (CASANTHRANOLDOCUSATE SODIUM:Psoriasis:1.37), (CASPOFUNGIN:Psoriasis:0.06), (CEFOTAXIMECEFOTAXIME SODIUM:Psoriasis:0.87), (CEFPROZIL:Psoriasis:1.08), (CEFTAZIDIME SODIUM:Psoriasis:0.04), (CERITINIB:Psoriasis:0.1), (CERIVASTATIN SODIUM:Psoriasis:0.61), (CHLORAMBUCIL:Psoriasis:0.44), (CHLORMADINONE ACETATE:Psoriasis:0.66), (CHLORPROTHIXENE:Psoriasis:0.48), (CISAPRIDE:Psoriasis:0.51), (CODEINE PHOSPHATEDICLOFENAC SODIUM:Psoriasis:0.57), (COLISTIMETHATE SODIUM:Psoriasis:0.1), (CRIZOTINIB:Psoriasis:0.33), (CYANOCOBALAMINDICLOFENAC SODIUMPYRIDOXINE HYDROCHLORIDETHIAMINE MONONITRATE:Psoriasis:0.5), (CYPROTERONE:Psoriasis:0.23), (DABRAFENIB MESYLATE:Psoriasis:0), (DANAZOL:Psoriasis:1.13), (DARIFENACIN HYDROBROMIDE:Psoriasis:0.61), (DEGARELIX:Psoriasis:0.42), (DEQUALINIUM CHLORIDE:Psoriasis:0.64), (DEXAMETHASONE ACETATE:Psoriasis:1.28), (DEXTROMETHORPHAN HYDROBROMIDEQUINIDINE SULFATE:Psoriasis:0.7), (DIENOGEST:Psoriasis:0.67), (DIETARY SUPPLEMENTRESVERATROL:Psoriasis:0.5), (DIHYDROERGOTAMINE MESYLATE:Psoriasis:0.32), (DILAZEP:Psoriasis:1.08), (DIMETHICONE 410:Psoriasis:0.38), (DIMETHYL SULFOXIDE:Psoriasis:0.23), (DIOSMIN:Psoriasis:0.84), (DIPYRONEPROMETHAZINE HYDROCHLORIDE:Psoriasis:2.16), (DISOPYRAMIDE:Psoriasis:0.17), (DISTIGMINE BROMIDE:Psoriasis:0.15), (DOCUSATE SODIUMSENNOSIDES A AND B:Psoriasis:0.06), (DOLASETRON MESYLATE:Psoriasis:1), (DOLUTEGRAVIR SODIUM:Psoriasis:0.79), (DOXORUBICIN HYDROCHLORIDE:Psoriasis:0.02), (DYDROGESTERONE:Psoriasis:0.51), (EDARAVONE:P soriasis:0), (EDOXABAN TOSILATE MONOHYDRATE:Psoriasis:0.2), (EDOXABAN TOSYLATE:Psoriasis:2.66), (ELOTUZUMAB:Psoriasis:0.52), (ERGOLOID MESYLATES:Psoriasis:0.03), (ERTAPENEM SODIUM:Psoriasis:0.78), (ERYTHROMYCIN LACTOBIONATE:Psoriasis:0.14), (ESTRADIOL CYPIONATE:Psoriasis:0.49), (ESTRADIOLLEVONORGESTREL:Psoriasis:0.85), (ESTROGE NSMETHYLTESTOSTERONE:Psoriasis:0.36), (ETHINYL ESTRADIOLETHYNODIOL:Psoriasis:0.17), (ETHOSUXIMIDE:Psoriasis:0.34), (EUCALYPT OLMENTHOLMETHYL SALICYLATETHYMOL:Psoriasis:0.64), (FERROUS SULFATE ANHYDROUS:Psoriasis:1.5), (FEXOFENADINE HYDROCHLORIDEPSEUDOEPHEDRINE HYDROCHLORIDE:Psoriasis:0.13), (FINGOLIMOD:Psoriasis:0.01), (FLAVOXATE HYDROCHLORIDE:Psoriasis:0.15), (FLUNARIZINE HYDROCHLORIDE:Psoriasis:1.15), (FLUOXETINE HYDROCHLORIDEOLANZAPINE:Psoriasis:0.76), (FONDAPARINUX:Psoriasis:0.6), (FOSP HENYTOINFOSPHENYTOIN SODIUM:Psoriasis:0.55), (FURSULTIAMINE:Psoriasis:0.18), (GADOBUTROL:Psoriasis:0.06), (GADOTERATE MEGLUMINE:Psoriasis:0.52), (GIMERACILOTERACILTEGAFUR:Psoriasis:0.51), (GLYCE RINMINERAL OILPARAFFIN:Psoriasis:0.13), (GLYCOPYRROLATEINDACATEROL MALEATE:Psoriasis:1.29), (GLYCOPYRRONIUM:Psoriasis:1.31), (GOLD SODIUM THIOMALATE:Psoriasis:0.13), (GREEN TEA LEAF:Psoriasis:0.28), (GUAIFENESINPHENYLEPHRINE HYDROCHLORIDE:Psoriasis:0.32), (HALOPERIDOL DECANOATE:Psoriasis:0.75), (HARPAGOPHYTUM PROCUMBENS ROOT:Psoriasis:0.06), (HEPARIN CALCIUM:Psoriasis:1.64), (HERBALSKARAYA GUM:Psoriasis:0.07), (HUMAN VARICELLA-ZOSTER IMMUNE GLOBULIN:Psoriasis:0.18), (HYDROCHLOROTHIAZIDEMETOPROLOL SUCCINATE:Psoriasis:0.26), (IDELALISIB:Psoriasis:0.37), (ILOPROST:Psoriasis:0.67), (IMID APRIL:Psoriasis:0.64), (IMIGLUCERASE:Psoriasis:1.17), (IMIPRAMINE PAMOATE:Psoriasis:0.06), (IMMUNE GLOBULIN NOS:Psoriasis:0.19), (INDACATEROL MALEATE:Psoriasis:0.31), (INOTUZUMAB OZOGAMICIN:Psoriasis:0.11), (INSULIN ASPARTINSULIN DEGLUDEC:Psoriasis:0.18), (INTERFERON ALFA-2A:Psoriasis:1.62), (INTERFERON BETA:Psoriasis:0.04), (IPRAGLIFLOZIN L-PROLINE:Psoriasis:0.13), (IRINOTECAN HYDROCHLORIDE:Psoriasis:0.89), (IRONPOLYSACCHARIDES:Psoriasis:0.89), (ITOPRID E:Psoriasis:0.14), (ITRACONAZOLE\SODIUM CHLORIDE:Psoriasis:0.61), (IXAZOMIB:Psoriasis:0.6), (LANREOTIDE:Psoriasis:0.89), (LANR EOTIDE ACETATE:Psoriasis:0.74), (LAPATINIB DITOSYLATE:Psoriasis:0.86), (LAVENDER OIL:Psoriasis:0.07), (LENOGRASTIM:Psoriasis:1.05), (LENVATINIB:Psoriasis:0.07), (LEVOL EUCOVORIN CALCIUM:Psoriasis:0.18), (LEVOMEFOLATE CALCIUM\METHYLCOBALAMINPYRIDOXAL 5-PHOSPHATE:Psoriasis:0.1), (LINSIDOMINE:Psoriasis:0), (LURASIDONE:Psoriasis:0.01), (LU TETIUM LU-177:Psoriasis:0.89), (LYSOZYME HYDROCHLORIDE:Psoriasis:0.51), (MAGALDRATE:Psoriasis:0.28), (MAGNESIUM GLYCINATE:Psoriasis:0.01), (MAGNESIUM PIDOLATE:Psoriasis:0.05), (MAGNESIUMPYRIDOXINE:Psoriasis:0.21), (MANIDIPINE HYDROCHLORIDE:Psoriasis:0.29), (MAPROTILINE HYDROCHLORIDE:Psoriasis:0.66), (MARAVIROC:Psoriasis:0.03), (MECLIZINE MONOHYDROCHLORIDENIACIN:Psoriasis:0.29), (MECLOZINE DIHYDROCHLORIDE:Psoriasis:0.14), (MELPERONE:Psoriasis:1.1), (MELPHALAN:Psoriasis: 0.5), (METHENAMINE MANDELATE:Psoriasis:1.08), (METHENOLONE ENANTHATE:Psoriasis:0.19), (METHYL SALICYLATE:Psoriasis:0.04), (METRELEPTIN:Psoriasis:1.17), (MEXILETINE HYDROCHLORIDE:Psoriasis:0.37), (MICAFUNGIN:Psoriasis:0.54), (MIPOMERSEN SODIUM:Psoriasis:0.2), (MITIGLINIDE CALCIUM HYDRATEVOGLIBOSE:Psoriasis:0.33), (MOSAPRIDE CITRATE DIHYDRATE:Psoriasis:0.08), (NABIXIMOLS:Psoriasis:0.19), (NALOXONE:Psoriasis:0.77), (N ALTREXONE HYDROCHLORIDE:Psoriasis:0.56), (NEOMYCINPOLYMYXIN:Psoriasis:0.04), (NIRAPARI B:Psoriasis:0.56), (NOREPINEPHRINE BITARTRATE:Psoriasis:0.51), (NORETHINDRONE ACETATE:Psoriasis:0.25), (OCRELIZUMAB:Psoriasis:0.17), (OLANZAPINE PAMOATE:Psoriasis:0.15), (OLAPARIB:Psoriasis:0.04), (OLODATEROL HYDROCHLORIDE:Psoriasis:0.48), (OLODATEROL HYDROCHLORIDETIOTROPIUM BROMIDE MONOHYDRATE:Psoriasis:1.83), (OXITRIPTAN:Psoriasis:0.42), (OXYBUTYNINOXYBUT YNIN CHLORIDE:Psoriasis:1.87), (PALONOSETRON HYDROCHLORIDE:Psoriasis:0.79), (PANTETHINE:Psoriasis:0.04), (PARATHYROID HORMONE:Psoriasis:0.2), (PARSLEY:Psoriasis:0.03), (PASIREOTIDE:Psoriasis:0.58), (PATIR OMER:Psoriasis:0.79), (PAZOPANIB HYDROCHLORIDE:Psoriasis:2.13), (PEGINTERFERON ALFA-2A OR PEGINTERFERON ALFA-2B:Psoriasis:0.28), (PEGINTERFERON BETA-1A:Psoriasis:2.31), (PEMETREXED DISODIUM:Psoriasis:1.08), (PENTAMIDINE:Psoriasis:0.21), (PEPSIN:Psoriasis:0.48), (PHENY LBUTAZONE:Psoriasis:0.21), (PHENYLEPHRINE HYDROCHLORIDEWITCH HAZEL:Psoriasis:0.34), (PHLOROGLUCINOL1,3,5-TRIMETHOXYBENZENE:Psoriasis:0.62), (PILSICAINIDE HYDROCHLORIDE:Psoriasis:0.78), (PIMOBENDAN:Psoriasis:0.67), (PIRBUTEROL ACETATE:Psoriasis:0.67), (PITOFENONE:Psoriasis:0.02), (PONATINIB:Psoriasis:1.22), (POT ASSIUM CITRATETRISODIUM CITRATE DIHYDRATE:Psoriasis:0.4), (PRANLUKAST:Psoriasis:1.12), (PREDNISOLONE ACETATESULFACETAMIDE SODIUM:Psoriasis:0.06), (PREDNISOLONE SODIUM SUCCINATE:Psoriasis:1.27), (PRILOCAINE:Psoriasis:0.04), (PROBUCOL:Psoriasis:0.27), (PR OCAINE:Psoriasis:0.06), (PROTRIPTYLINE HYDROCHLORIDE:Psoriasis:1.65), (PRUCALOPRIDE SUCCINATE:Psoriasis:0.77), (PYRIMETHAMINE:Psoriasis:0.49), (QUINACRINE:Psoriasis:2.13), (QUINIDINE GLUCONATE:Psoriasis:0.98), (RED YEAST:Psoriasis:0.24), (RIFABUTIN:Psoriasis:0.43), (RILPIVIRINE HYDROCHLORIDE:Psoriasis:1.26), (RIOCIGUAT:Psoriasis:0.25), (RISEDRONIC ACID:Psoriasis:0.31), (SAFFLOWER OIL:Psoriasis:0.06), (SAPROPTERIN DIHYDROCHLORIDE:Psoriasis:0.6), (SERRAPEPTASE:Psoriasis:1.14), (SIBUTRAMINE HYDROCHLORIDE:Psoriasis:1.26), (SODIUM:Psoriasis:0.29), (SODIUM BICARBONATE\SODIUM CHLORIDE:Psoriasis:0.84), (SODIUM CITRATE:Psoriasis:0.07), (SODIUM MONOFLUOROPHOSPHATE:Psoriasis:0.07), (SODIUM PHOSPHATE, DIBASIC\SODIUM PHOSPHATE, MONOBASIC:Psoriasis:1.05), (STREPTOMYCIN SULFATE:Psoriasis:0.06), (SULFACETAMIDE SODIUMSULFUR:Psoriasis:1.34), (TAMOXIFEN CITRATE:Psoriasis:0.62), (TAURINE:Psoriasis:0.47), (TAZOBACTAM:Psoriasis:0.6), (TEDUG LUTIDE\WATER:Psoriasis:1.01), (TEGASEROD MALEATE:Psoriasis:0.87), (TELOTRISTAT ETHYL:Psoriasis:0.39), (TEMOCAPRIL HYDROCHLORIDE:Psoriasis:0.22), (TENOXICAM:Psoriasis:0.73), (TESAMORELIN:Psoriasi s:0.93), (THEOPHYLLINE:Psoriasis:0.65), (THIOTHIXENE:Psoriasis:0.05), (TIAGABINE HYDROCHLORIDE:Psoriasis:0.29), (TIANEPTINE:Psoriasis:0.25), (TIMOLOLTRAVOPROS T:Psoriasis:0.66), (TINIDAZOLE:Psoriasis:0.72), (TINZAPARIN:Psoriasis:1.2), (TIPIRACIL HYDROCHLORIDETRIFLURIDINE:Psoriasis:0.11), (TOFISOPAM:Psoriasis:0.03), (TRANY LCYPROMINE SULFATE:Psoriasis:0.28), (TRIMIPRAMINE MALEATE:Psoriasis:1.64), (TULOBUTEROL:Psoriasis:0.41), (VANDETANIB:Psoriasis:0.09), (VELAGLUCERASE ALFA:Psoriasis:0.02), (VEMURAFENIB:Psoriasis:1.32), (VINCRISTINE SULFATE:Psoriasis:0.43), (VINORELBINE TARTRATE:Psoriasis:0.3), (VONOPRAZAN FUMURATE:Psoriasis:0.21), (ZEAXANTHIN:Psoriasis:0.42), (ZICONOTIDE ACETATE:Psoriasis:0.76), (ZUCLOPENTHIXOL:Psoriasis:0), (.ALPHA.1-PROTEINASE INHIBITOR HUMAN:Psoriatic arthropathy:0.63), (ABOBOTULINUMTOXINA:Psoriatic arthropathy:0.77), (ACETAMINOPHENBUTALBITALCAFFEINECODEINE PHOSPHATE:Psoriatic arthropathy:0.34), (ACETYLCARNITINE HYDROCHLORIDE:Psoriatic arthropathy:0.6), (ACTIVATED CHARCOAL:Psoriatic arthropathy:0.18), (ADO-TRASTUZUMAB EMTANSINE:Psoriatic arthropathy:0.22), (AGALSIDASE BETA:Psoriatic arthropathy:1.7), (ALEMTUZUMAB:Psoriatic arthropathy:0.62), (ALFALFA:Psoriatic arthropathy:0.28), (ALMOTRIPTAN MALATE:Psoriatic arthropathy:0.44), (ALPROSTADIL:Psoriatic arthropathy:0.14), (ALUMINUM HYDROXIDEMAGNESIUM HYDROXIDESIMETHICONE:Psoriatic arthropathy:0.14), (AMILORIDE HYDROCHLORIDEFUROSEMIDE:Psoriatic arthropathy:0.38), (AMINO ACIDSDEXTROSEELECTROLYTES NOS:Psoriatic arthropathy:0.74), (AMINOPHYLLINE:Psoriatic arthropathy:0.02), (AMINOSALICYLIC ACID:Psoriatic arthropathy:0.09), (AMOXICILLINCLARITHROMYCINESOMEPRAZOLE:Psoriatic arthropathy:0.81), (ANAGRELIDE HYDROCHLORIDE:Psoriatic arthropathy:0.2), (ARIPIPRAZOLE LAUROXIL:Psoriatic arthropathy:0.6), (ASCORBIC ACIDBIOTINCYANOCOBALAMINFOLIC ACIDNIACINPANTOTHENIC ACIDPYRIDOXINE\RIBOFLAVINTHIAMINE:Psoriatic arthropathy:0.38), (ASPIRIN\CAFFEINESALICYLAMIDE:Psoriatic arthropathy:0.25), (BETAMETHASONE DIPROPIONATEBETAMETHASONE SODIUM PHOSPHATE:Psoriatic arthropathy:0.16), (LACTIS:Psoriatic arthropathy:0.25), (BIMATOPROSTTIMOLOL:Psoriatic arthropathy:0.45), (BOCEPREVIR:Psoriatic arthropathy:0.22), (BOTULINUM TOXIN TYPE A:Psoriatic arthropathy:0.82), (BUDESONIDEFORMOTEROL:Psoriatic arthropathy:0.64), (CABOZANTINIB S-MALATE:Psoriatic arthropathy:0.06), (CAFFEINEISOMETHEPTENEMETHIMAZOLE:Psoriatic arthropathy:0.14), (CALCIUM BICARBONATE:Psoriatic arthropathy:0.06), (CALCIUM CARBONATEMAGNESIUM HYDROXIDE:Psoriatic arthropathy:0.14), (CALCIUM PANTOTHENATECYANOCOBALAMINNIACINAMIDEPYRIDOXINE HYDROCHLORIDE\RIBOFLAVINTHIAMINE HYDROCHLORIDE:Psoriatic arthropathy:0.04), (CAMPHORDIPHENHYDRAMINE\ZINC OXIDE:Psoriatic arthropathy:0.13), (CARBIMAZOLE:Psoriatic arthropathy:1.08), (CARFILZOMIB:Psoriatic arthropathy:1.02), (CARTEOLOL HYDROCHLORIDE:Psoriatic arthropathy:0.39), (CASANTHRANOLDOCUSATE SODIUM:Psoriatic arthropathy:1.12), (CEFEPIMECEFEPIME HYDROCHLORIDE:Psoriatic arthropathy:0.43), (CEFOTAXIME SODIUM:Psoriatic arthropathy:1.43), (CEFOTAXIMECEFOTAXIME SODIUM:Psoriatic arthropathy:0.12), (CEFPODOXIME PROXETIL:Psoriatic arthropathy:0.81), (CERIVASTATIN SODIUM:Psoriatic arthropathy:0.92), (CICLOPIROX OLAMINE:Psoriatic arthropathy:0.43), (CLIKSTAR:Psoriatic arthropathy:0.96), (CLOBAZAM:Psoriatic arthropathy:0.93), (CLOCORTOLONE PIVALATE:Psoriatic arthropathy:0.07), (CODEINE PHOSPHATEDICLOFENAC SODIUM:Psoriatic arthropathy:0.61), (COLISTIMETHATE SODIUM:Psoriatic arthropathy:0.08), (COUMARINTROXERUTIN:Psoriatic arthropathy:0.03), (CYANOCOBALAMINDICLOFENAC SODIUMPYRIDOXINE HYDROCHLORIDETHIAMINE MONONITRATE:Psoriatic arthropathy:0.44), (CYPROTERONE ACETATE:Psoriatic arthropathy:0.14), (CYPROTERONE ACETATEETHINYL ESTRADIOL:Psoriatic arthropathy:0.47), (DACLIZUMAB:Psoriatic arthropathy:2.01), (DARIFENACIN HYDROBROMIDE:Psoriatic arthropathy:0.62), (DASABUVIROMBITASVIRPARITAPREVIRRITONAVIR:Psoriatic arthropathy:1.15), (DASATINIB:Psoriatic arthropathy:1.48), (DEXAMETHASONE ACETATE:Psoriatic arthropathy:0.08), (DIENOGEST:Psoriatic arthropathy:0.32), (DIHYDROERGOTAMINE MESYLATE:Psoriatic arthropathy:0.01), (DIPYRONEPROMETHAZINE HYDROCHLORIDE:Psoriatic arthropathy:1.79), (DOLASETRON MESYLATE:Psoriatic arthropathy:0.41), (ELOTUZUMAB:Psoriatic arthropathy:0.09), (ERTAPENEM SODIUM:Psoriatic arthropathy:0.53), (ESTRADIOL ACETATE:Psoriatic arthropathy:0.56), (ESTRADIOL BENZOATE:Psoriatic arthropathy:0.43), (ESTRADIOL HEMIHYDRATE:Psoriatic arthropathy:0.19), (ESTRADIOLLEVONORGESTREL:Psoriatic arthropathy:0.3), (ETHACRYNIC ACID:Psoriatic arthropathy:0.92), (EUCALYPTOLMENTHOLMETHYL SALICYLATETHYMOL:Psoriatic arthropathy:0.34), (EVEROLIMUS:Psoriatic arthropathy:1.48), (FENOTEROL HYDROBROMIDEIPRATROPIUM BROMIDE:Psoriatic arthropathy:0.86), (FEXOFENADINE HYDROCHLORIDEPSEUDOEPHEDRINE HYDROCHLORIDE:Psoriatic arthropathy:0.04), (FLAVOXATE HYDROCHLORIDE:Psoriatic arthropathy:0.53), (FLUNARIZINE HYDROCHLORIDE:Psoriatic arthropathy:0.72), (FLUPHENAZINE:Psoriatic arthropathy:0.02), (FLUPIRTINE:Psoriatic arthropathy:0.06), (FONDAPARINUX:Psoriatic arthropathy:0.62), (FORMOTEROL FUMARATE DIHYDRATE:Psoriatic arthropathy:0.47), (FORMOTEROL FUMARATE\MOMETASONE FUROATE:Psoriatic arthropathy:1.41), (FOSFOMYCIN:Psoriatic arthropathy:0.46), (FOSPHENYTOINFOSPHENYTOIN SODIUM:Psoriatic arthropathy:0.12), (GLYCERIN:Psoriatic arthropathy:0.35), (GLYCERINPARAFFIN:Psoriatic arthropathy:0.11), (GLYCOPYRROLATEINDACATEROL MALEATE:Psoriatic arthropathy:0.97), (GLYCOPYRRONIUM:Psoriatic arthropathy:0.92), (GREEN TEA LEAF:Psoriatic arthropathy:0.13), (GUAIFENESINPHENYLEPHRINE HYDROCHLORIDE:Psoriatic arthropathy:0.07), (HALOPERIDOL DECANOATE:Psoriatic arthropathy:0.1), (HYDROCHLOROTHIAZIDEMETOPROLOL SUCCINATE:Psoriatic arthropathy:0.36), (HYDROXYCITRIC ACID:Psoriatic arthropathy:0.5), (ILOPROST:Psoriatic arthropathy:0.92), (IMIGLUCERASE:Psoriatic arthropathy:0.71), (IMIPRAMINE PAMOATE:Psoriatic arthropathy:0), (INDACATEROL MALEATE:Psoriatic arthropathy:0.18), (INSULIN PORKINSULIN PURIFIED PORK:Psoriatic arthropathy:0.74), (INTERFERON ALFA-2A:Psoriatic arthropathy:0.86), (INTERFERON GAMMA-1B:Psoriatic arthropathy:0.29), (ISOPROPYL ALCOHOLLEUPROLIDE ACETATENORETHINDRONE ACETATE:Psoriatic arthropathy:0.64), (ITRACONAZOLE\SODIUM CHLORIDE:Psoriatic arthropathy:0.28), (IXAZOMIB:Psoriatic arthropathy:0.18), (KETOTIFEN:Psoriatic arthropathy:0.19), (LANREOTIDE:Psoriatic arthropathy:0.47), (LANREOTIDE ACETATE:Psoriatic arthropathy:0.34), (LAPATINIB DITOSYLATE:Psoriatic arthropathy:0.14), (LENOGRASTIM:Psoriatic arthropathy:0.07), (LIDOCAINEPRILOCAINE:Psoriatic arthropathy:0.91), (LOMITAPIDE MESYLATE:Psoriatic arthropathy:1.41), (LUTETIUM LU-177:Psoriatic arthropathy:0.53), (MAGNESIUM ASPARTATE HYDROCHLORIDE:Psoriatic arthropathy:0.78), (MAPROTILINE HYDROCHLORIDE:Psoriatic arthropathy:0.07), (MECLIZINE MONOHYDROCHLORIDENIACIN:Psoriatic arthropathy:0.06), (MECLOZINE DIHYDROCHLORIDE:Psoriatic arthropathy:0.02), (MEGESTROL ACETATE:Psoriatic arthropathy:0.85), (MELPERONE:Psoriatic arthropathy:0.51), (METRELEPTIN:Psoriatic arthropathy:0.62), (MIDODRINE HYDROCHLORIDE:Psoriatic arthropathy:1.15), (MIGLITOL:Psoriatic arthropathy:0.25), (MILNACIPRAN:Psoriatic arthropathy:0.14), (NAFTIFINE HYDROCHLORIDE:Psoriatic arthropathy:0.25), (NALBUPHINE HYDROCHLORIDE:Psoriatic arthropathy:0.08), (NALOXONE:Psoriatic arthropathy:0.47), (NEFAZODONE HYDROCHLORIDE:Psoriatic arthropathy:0.46), (NEOMYCIN:Psoriatic arthropathy:0.95), (NILOTINIB:Psoriatic arthropathy:2.01), (NIMODIPINE:Psoriatic arthropathy:0.02), (NIRAPARIB:Psoriatic arthropathy:0.02), (NOREPINEPHRINENOREPINEPHRINE BITARTRATE:Psoriatic arthropathy:0.09), (OCTREOTIDE ACETATE:Psoriatic arthropathy:0.76), (OXYBUTYNINOXYBUTYNIN CHLORIDE:Psoriatic arthropathy:1.57), (PALBOCICLIB:Psoriatic arthropathy:0.18), (PALONOSETRON HYDROCHLORIDE:Psoriatic arthropathy:0.02), (PARATHYROID HORMONE:Psoriatic arthropathy:0.01), (PASIREOTIDE:Psoriatic arthropathy:0.44), (PATIROMER:Psoriatic arthropathy:0.67), (PEGFILGRASTIM:Psoriatic arthropathy:0.18), (PEMETREXED DISODIUM:Psoriatic arthropathy:0.36), (PEPSIN:Psoriatic arthropathy:0.16), (PHENELZINE SULFATE:Psoriatic arthropathy:0.1), (PHENYLEPHRINE HYDROCHLORIDEPROMETHAZINE HYDROCHLORIDE:Psoriatic arthropathy:0.05), (PHENYLEPHRINE HYDROCHLORIDEWITCH HAZEL:Psoriatic arthropathy:0.11), (PHTHALYLSULFATHIAZOLE:Psoriatic arthropathy:0.06), (PIRBUTEROL ACETATE:Psoriatic arthropathy:0.49), (POVIDONE:Psoriatic arthropathy:0.83), (PRAMOXINE HYDROCHLORIDE:Psoriatic arthropathy:1.01), (PRANLUKAST:Psoriatic arthropathy:0.13), (PREDNISOLONE ACETATESULFACETAMIDE SODIUM:Psoriatic arthropathy:0.02), (PREDNISOLONE SODIUM PHOSPHATE:Psoriatic arthropathy:0.81), (PREDNISOLONE SODIUM SUCCINATE:Psoriatic arthropathy:0.06), (PROPATYL NITRATE:Psoriatic arthropathy:0.79), (PROTRIPTYLINE HYDROCHLORIDE:Psoriatic arthropathy:1.66), (PRUCALOPRIDE SUCCINATE:Psoriatic arthropathy:0.58), (PYRIDOSTIGMINE:Psoriatic arthropathy:0), (QUINIDINE GLUCONATE:Psoriatic arthropathy:0.38), (RADIATION THERAPY:Psoriatic arthropathy:0.03), (RIFABUTIN:Psoriatic arthropathy:0.06), (RILPIVIRINE HYDROCHLORIDE:Psoriatic arthropathy:0.73), (RIVASTIGMINE:Psoriatic arthropathy:1.63), (SAFFLOWER OIL:Psoriatic arthropathy:0.15), (SERRAPEPTASE:Psoriatic arthropathy:0.5), (SODIUM BICARBONATE\SODIUM CHLORIDE:Psoriatic arthropathy:0.53), (SOFOSBUVIRVELPATASVIR:Psoriatic arthropathy:0.75), (SUPLATAST TOSILATE:Psoriatic arthropathy:0.01), (TAURINE:Psoriatic arthropathy:0.21), (TAZOBACTAM:Psoriatic arthropathy:0.23), (TEDUGLUTIDE\WATER:Psoriatic arthropathy:0.45), (TEGASEROD MALEATE:Psoriatic arthropathy:0.54), (TESAMORELIN:Psoriatic arthropathy:0.68), (TESTOSTERONE ENANTHATE:Psoriatic arthropathy:0.05), (TESTOSTERONE UNDECANOATE:Psoriatic arthropathy:1.73), (TETRABENAZINE:Psoriatic arthropathy:0.25), (THEOPHYLLINE:Psoriatic arthropathy:0.6), (TIAGABINE HYDROCHLORIDE:Psoriatic arthropathy:0.18), (TIMOLOLTRAVOPROST:Psoriatic arthropathy:0.63), (TINZAPARIN:Psoriatic arthropathy:0.2), (TINZAPARIN SODIUM:Psoriatic arthropathy:0.17), (TIOTROPIUM:Psoriatic arthropathy:1.51), (TRANYLCYPROMINE SULFATE:Psoriatic arthropathy:0.06), (TREPROSTINIL DIOLAMINE:Psoriatic arthropathy:0.09), (TRIPTORELIN PAMOATE:Psoriatic arthropathy:0.85), (TRYPTOPHAN:Psoriatic arthropathy:0.25), (VALGANCICLOVIR HYDROCHLORIDE:Psoriatic arthropathy:1.62), (VANCOMYCIN HYDROCHLORIDE:Psoriatic arthropathy:1.02), (VISMODEGIB:Psoriatic arthropathy:0.21), (ZEAXANTHIN:Psoriatic arthropathy:0.28), (ZICONOTIDE ACETATE:Psoriatic arthropathy:0.45), (ZILEUTON:Psoriatic arthropathy:1.08), (ZIPRASIDONE HYDROCHLORIDE:Psoriatic arthropathy:0.14), (OXCARBAZEPINE:Psychiatric symptom:0.17), (VARENICLINE TARTRATE:Psychiatric symptom:0.04), (ALCOHOL:Psychomotor hyperactivity:0.45), (ARMODAFINIL:Psychomotor hyperactivity:0.1), (BUPRENORPHINE HYDROCHLORIDE:Psychomotor hyperactivity:0.15), (CERTOLIZUMAB PEGOL:Psychomotor hyperactivity:0.19), (ESZOPICLONE:Psychomotor hyperactivity:0.01), (EVOLOCUMAB:Psychomotor hyperactivity:0.1), (METHYLPREDNISOLONE SODIUM SUCCINATE:Psychomotor hyperactivity:0.23), (METRONIDAZOLE:Psychomotor hyperactivity:0.5), (PANTOPRAZOLE:Psychomotor hyperactivity:0.26), (PRAMIPEXOLE DIHYDROCHLORIDE:Psychomotor hyperactivity:0.09), (PRAVASTATIN SODIUM:Psychomotor hyperactivity:0.04), (RANITIDINE HYDROCHLORIDE:Psychomotor hyperactivity:0.42), (SACUBITRILVALSARTAN:Psychomotor hyperactivity:0.04), (TERIFLUNOMIDE:Psychomotor hyperactivity:0.49), (WARFARIN:Psychomotor hyperactivity:0.52), (ABACAVIR:Psychotic disorder:0.26), (ABACAVIR SULFATEDOLUTEGRAVIR SODIUMLAMIVUDINE:Psychotic disorder:0.37), (ACARBOSE:Psychotic disorder:0.56), (ACETAMINOPHENBUTALBITALCAFFEINE:Psychotic disorder:1.1), (ACETAMINOPHENHYDROCODONE:Psychotic disorder:0.31), (ACETAMINOPHENOXYCODONEOXYCODONE HYDROCHLORIDE:Psychotic disorder:0.78), (ADO-TRASTUZUMAB EMTANSINE:Psychotic disorder:0.19), (AGALSIDASE BETA:Psychotic disorder:0), (ALISKIREN:Psychotic disorder:0.21), (AMINOPYRINEANTIPYRINECAFFEINEPHENACETIN:Psychotic disorder:0.05), (AMLODIPINE BESYLATEHYDROCHLOROTHIAZIDEVALSARTAN:Psychotic disorder:0.13), (AMLODIPINEPERINDOPRIL:Psychotic disorder:0.09), (AMLODIPINEVALSARTAN:Psychotic disorder:0.17), (AMOBARBITAL SODIUM:Psychotic disorder:0.36), (AMPHETAMINE ADIPATEDEXTROAMPHETAMINE:Psychotic disorder:0.4), (AMPHETAMINEDEXTROAMPHETAMINE:Psychotic disorder:0.01), (AMPHOTERICIN B:Psychotic disorder:0.35), (BENAZEPRILBENAZEPRIL HYDROCHLORIDE:Psychotic disorder:0.43), (BRINZOLAMIDE:Psychotic disorder:0.04), (BRIVARACETAM:Psychotic disorder:0.45), (BUDESONIDEFORMOTEROL FUMARATE:Psychotic disorder:0.35), (BUPRENORPHINENALOXONE:Psychotic disorder:0.99), (CANAGLIFLOZINMETFORMIN HYDROCHLORIDE:Psychotic disorder:0.21), (CANNABIS SATIVA SUBSP. SATIVA FLOWERING TOP:Psychotic disorder:0.36), (CARBIDOPAMELEVODOPA:Psychotic disorder:1.14), (CARBIMAZOLE:Psychotic disorder:0.29), (CARFILZOMIB:Psychotic disorder:0.28), (CELERY SEED:Psychotic disorder:0.36), (CHLORDIAZEPOXIDE:Psychotic disorder:0.7), (CHOLESTYRAMINE:Psychotic disorder:1.22), (CHONDROITIN SULFATE (BOVINE)GLUCOSAMINE HYDROCHLORIDE:Psychotic disorder:0.21), (CICLESONIDE:Psychotic disorder:0.02), (CIPROFLOXACIN HYDROCHLORIDE:Psychotic disorder:0.24), (CLOPENTHIXOL HYDROCHLORIDE:Psychotic disorder:0.04), (COLESEVELAM HYDROCHLORIDE:Psychotic disorder:0.19), (DACLIZUMAB:Psychotic disorder:0.32), (DALTEPARIN:Psychotic disorder:0.63), (DEFLAZACORT:Psychotic disorder:0.24), (DESLORATADINE:Psychotic disorder:1.3), (DESONIDE:Psychotic disorder:0.19), (DEUTETRABENAZINE:Psychotic disorder:0.37), (DEXMETHYLPHENIDATE HYDROCHLORIDE:Psychotic disorder:0.42), (DIACETYLMORPHINE:Psychotic disorder:0.77), (DIAMORPHINE:Psychotic disorder:0.83), (DIGITOXIN:Psychotic disorder:0.14), (DIPYRONE:Psychotic disorder:0.07), (DOFETILIDE:Psychotic disorder:0.59), (DOLUTEGRAVIR:Psychotic disorder:0.03), (DOXYLAMINE SUCCINATE:Psychotic disorder:0.19), (DULAGLUTIDE:Psychotic disorder:0.07), (DUTASTERIDETAMSULOSIN HYDROCHLORIDE:Psychotic disorder:0.47), (EDOXABAN TOSYLATE:Psychotic disorder:0.63), (EFAVIRENZEMTRICITABINETENOFOVIR DISOPROXIL FUMARATE:Psychotic disorder:0.63), (ELETRIPTAN HYDROBROMIDE:Psychotic disorder:1.28), (EMTRICITABINERILPIVIRINE HYDROCHLORIDETENOFOVIR DISOPROXIL FUMARATE:Psychotic disorder:0.02), (ENALAPRIL MALEATEHYDROCHLOROTHIAZIDE:Psychotic disorder:0.52), (ESTROGENS, CONJUGATEDMEDROXYPROGESTERONE ACETATE:Psychotic disorder:0.64), (ESTRONESUS SCROFA OVARY:Psychotic disorder:0.09), (ETORICOXIB:Psychotic disorder:0.79), (EVEROLIMUS:Psychotic disorder:1.18), (EVOLOCUMAB:Psychotic disorder:1.07), (FENOFIBRIC ACID:Psychotic disorder:0.11), (FENOTEROL HYDROBROMIDEIPRATROPIUM BROMIDE:Psychotic disorder:0.02), (FLUPENTIXOL DIHYDROCHLORIDE:Psychotic disorder:0.55), (FLUTICASONE FUROATEVILANTEROL TRIFENATATE:Psychotic disorder:0.12), (FORMOTEROL FUMARATE:Psychotic disorder:0.48), (FOSPHENYTOINFOSPHENYTOIN SODIUM:Psychotic disorder:0.11), (GENTAMICIN:Psychotic disorder:0.2), (GLYCOPYRRONIUMINDACATEROL:Psychotic disorder:0.83), (HERBALSSAW PALMETTO:Psychotic disorder:0.12), (HERBALSTURMERIC:Psychotic disorder:0.03), (HYDROCHLOROTHIAZIDETELMISARTAN:Psychotic disorder:0.67), (HYDROXYCHLOROQUINE DIPHOSPHATE:Psychotic disorder:0.02), (HYOSCYAMINE:Psychotic disorder:0.03), (ILOPROST:Psychotic disorder:0.03), (IMATINIB MESYLATE:Psychotic disorder:0.79), (INDAPAMIDEPERINDOPRIL:Psychotic disorder:1.73), (INDOMETHACIN:Psychotic disorder:0.49), (INSULIN BEEF:Psychotic disorder:0.04), (ISONIAZIDPYRAZINAMIDERIFAMPIN:Psychotic disorder:0.22), (ISTRADEFYLLINE:Psychotic disorder:1.24), (KETOPROFEN:Psychotic disorder:0.23), (LACTOBACILLUS RHAMNOSUS:Psychotic disorder:0.11), (LIDOCAINE HYDROCHLORIDE:Psychotic disorder:0.26), (LIDOCAINEPRILOCAINE:Psychotic disorder:0.09), (LINAGLIPTIN:Psychotic disorder:1.03), (LIOTHYRONINE SODIUM:Psychotic disorder:0.25), (LISDEXAMFETAMINE:Psychotic disorder:0.69), (LOPRAZOLAM:Psychotic disorder:0.01), (MECLIZINEMECLIZINE HYDROCHLORIDE:Psychotic disorder:0.17), (MEGESTROL ACETATE:Psychotic disorder:0.34), (MEPERIDINE:Psychotic disorder:0.56), (METFORMIN HYDROCHLORIDEVILDAGLIPTIN:Psychotic disorder:0.3), (METHENAMINE HIPPURATE:Psychotic disorder:0.68), (METHYLENEDIOXYMETHAMPHETAMINE:Psychotic disorder:0.35), (METHYLPREDNISOLONE ACETATE:Psychotic disorder:0.69), (MORPHINE\MORPHINE HYDROCHLORIDE\MORPHINE SULFATE\MORPHINE TARTRATE:Psychotic disorder:0.88), (MOXIFLOXACIN HYDROCHLORIDE:Psychotic disorder:1.3), (MOXONIDINE:Psychotic disorder:0.63), (NABUMETONE:Psychotic disorder:0.38), (NALOXONE HYDROCHLORIDETILIDINE HYDROCHLORIDE:Psychotic disorder:0.15), (NATEGLINIDE:Psychotic disorder:0.12), (NIAOULI OILQUININE BENZOATETHIAMINE HYDROCHLORIDE:Psychotic disorder:0.19), (NOREPINEPHRINE:Psychotic disorder:0.02), (OCTREOTIDE ACETATE:Psychotic disorder:0.12), (OLMESARTAN:Psychotic disorder:0.2), (OLODATEROL HYDROCHLORIDETIOTROPIUM BROMIDE MONOHYDRATE:Psychotic disorder:0.11), (OPICAPONE:Psychotic disorder:0.22), (OSELTAMIVIR PHOSPHATE:Psychotic disorder:0.42), (OXYMORPHONE:Psychotic disorder:0.62), (PAMIDRONATE DISODIUM:Psychotic disorder:0.06), (PANCRELIPASE AMYLASEPANCRELIPASE LIPASEPANCRELIPASE PROTEASE:Psychotic disorder:0.14), (PARICALCITOL:Psychotic disorder:0.03), (PAZOPANIB HYDROCHLORIDE:Psychotic disorder:1), (PEGINTERFERON BETA-1A:Psychotic disorder:0.35), (PEMETREXED DISODIUM:Psychotic disorder:0.27), (PERGOLIDE:Psychotic disorder:0.34), (PEROSPIRONE:Psychotic disorder:1.05), (PIROXICAM:Psychotic disorder:0.27), (PITAVASTATIN CALCIUM:Psychotic disorder:0.08), (PONATINIB:Psychotic disorder:0.45), (PREDNISOLONEPREDNISOLONE ACETATEPREDNISOLONE SODIUM SUCCINATE:Psychotic disorder:0.12), (PRISTINAMYCIN:Psychotic disorder:0), (QUINAGOLIDE HYDROCHLORIDE:Psychotic disorder:0.64), (RABEPRAZOLE SODIUM:Psychotic disorder:0.76), (RILMENIDINE PHOSPHATE:Psychotic disorder:0.1), (RIZATRIPTAN BENZOATE:Psychotic disorder:0.97), (SACUBITRILVALSARTAN:Psychotic disorder:0.68), (SECUKINUMAB:Psychotic disorder:1.19), (ST. JOHN'S WORT:Psychotic disorder:0.3), (SULFAMETHOXAZOLE:Psychotic disorder:0.19), (SULTHIAME:Psychotic disorder:0.11), (SUMATRIPTANSUMATRIPTAN SUCCINATE:Psychotic disorder:1.28), (TERBINAFINE HYDROCHLORIDE:Psychotic disorder:0.61), (TERBUTALINE SULFATE:Psychotic disorder:1.01), (THALIDOMIDE:Psychotic disorder:0.42), (TIMOLOL:Psychotic disorder:0.84), (TOCOPHEROL:Psychotic disorder:0.18), (TRIAMTERENE:Psychotic disorder:0.28), (TRIMIPRAMINE MALEATE:Psychotic disorder:1.08), (VALGANCICLOVIR HYDROCHLORIDE:Psychotic disorder:0.15), (VANCOMYCIN HYDROCHLORIDE:Psychotic disorder:0.18), (VITAMIN A:Psychotic disorder:0.53), (.ALPHA.-TOCOPHEROLVITAMINS:Pulmonary arterial hypertension:0.21), (ACEBUTOLOL HYDROCHLORIDE:Pulmonary arterial hypertension:0.43), (ACECLOFENAC:Pulmonary arterial hypertension:0.16), (ACETAMINOPHEN\CAFFEINEOPIUM:Pulmonary arterial hypertension:0.08), (ACETAMINOPHEN\CAFFEINEPROMETHAZINESALICYLAMIDE:Pu lmonary arterial hypertension:0.04), (ACITRETIN:Pulmonary arterial hypertension:0.81), (ADENOSINE TRIPHOSPHATE:Pulmonary arterial hypertension:0.14), (ADO-TRASTUZUMAB EMTANSINE:Pulmonary arterial hypertension:0.76), (AGALSIDASE BETA:Pulmonary arterial hypertension:1.36), (ALBIGLUTIDE:Pulmonary arterial hypertension:0.54), (ALBUTEROLGUAIFENESIN:Pulmonary arterial hypertension:0), (ALEMTUZUMAB:Pulmonary arterial hypertension:0.55), (ALGLUCOSIDASE ALFA:Pulmonary arterial hypertension:0.47), (AMIKACIN:Pulmonary arterial hypertension:0.78), (AMISULPRIDE:Pulmonary arterial hypertension:0.87), (AMOXICILLINCLARITHROMYCINESOMEPRAZOLE:Pulmonary arterial hypertension:0.28), (ANAGLIPTIN:Pulmonary arterial hypertension:0.08), (APREPITANT:Pulmonary arterial hypertension:0.53), (ASCORBIC ACID/CHROMIUM/SELENIUM/VANADYL SULFATE:Pulmonary arterial hypertension:0.51), (ASPIRIN\CAFFEINESALICYLAMIDE:Pulmonary arterial hypertension:0.79), (ASPIRINDIHYDROXYALUMINUM AMINOACETATE ANHYDROUSMAGNESIUM CARBONATE:Pulmonary arterial hypertension:0.89), (ASPIRINMAGNESIUM OXIDE:Pulmonary arterial hypertension:0.4), (ASUNAPREVIR:Pulmonary arterial hypertension:0.54), (ATAZANAVIR:Pulmonary arterial hypertension:0.03), (ATROPINE SULFATEHYOSCYAMINE SULFATEPHENOBARBITALSCOPOLAMINE HYDROBROMIDE:Pulmonary arterial hypertension:0.05), (AVANAFIL:Pulmonary arterial hypertension:0.29), (AXITINIB:Pulmonary arterial hypertension:0.75), (BALSALAZIDE DISODIUM:Pulmonary arterial hypertension:0.49), (BAMIFYLLINE HYDROCHLORIDE:Pulmonary arterial hypertension:0.32), (BECLOMETHASONE DIPROPIONATEFORMOTEROL:Pulmonary arterial hypertension:0.06), (BELATACEPT:Pulmonary arterial hypertension:0.09), (BETAHISTINE MESILATE:Pulmonary arterial hypertension:0.64), (BETAMETHASONE BUTYRATE PROPIONATE:Pulmonary arterial hypertension:0.01), (BETAMETHASONECALCIPOTRIENE:Pulmonary arterial hypertension:0.93), (BICARBONATE ION:Pulmonary arterial hypertension:0.24), (ACIDOPHILUS:Pulmonary arterial hypertension:0.19), (BIMATOPROSTTIMOLOL:Pulmonary arterial hypertension:0.24), (BOCEPREVIR:Pulmonary arterial hypertension:0.04), (BOCOCIZUMAB:Pulmonary arterial hypertension:0.43), (BRINZOLAMIDETIMOLOL MALEATE:Pulmonary arterial hypertension:0.44), (BUPIVACAINE HYDROCHLORIDE:Pulmonary arterial hypertension:0.06), (BUPRENORPHINE HYDROCHLORIDE:Pulmonary arterial hypertension:1.66), (BUTYLSCOPOLAMINE BROMIDE:Pulmonary arterial hypertension:1.06), (CAJUPUT OILEUCALYPTUS OILPEPPERMINT OIL:Pulmonary arterial hypertension:0.02), (CALCIUM\MAGNESIUM\ZINC:Pulmonary arterial hypertension:1.19), (CAMPHORMENTHOLMETHYL SALICYLATE:Pulmonary arterial hypertension:0.04), (CARFILZOMIB:Pulmonary arterial hypertension:1.93), (CARTEOLOL HYDROCHLORIDE:Pulmonary arterial hypertension:0.25), (CEFCAPENE PIVOXIL HYDROCHLORIDE:Pulmonary arterial hypertension:0.25), (CEFEPIMECEFEPIME HYDROCHLORIDE:Pulmonary arterial hypertension:0.72), (CEFOTAXIMECEFOTAXIME SODIUM:Pulmonary arterial hypertension:0.54), (CERIVASTATIN SODIUM:Pulmonary arterial hypertension:0.8), (CHLORAMPHENICOL:Pulmonary arterial hypertension:0.53), (CHOLESTEROL:Pulmonary arterial hypertension:0.02), (CHONDROITIN SULFATE (CHICKEN):Pulmonary arterial hypertension:0.2), (CHROMIUM PICOLINATE:Pulmonary arterial hypertension:0.08), (CIFENLINE:Pulmonary arterial hypertension:0.51), (CIPROFIBRATE:Pulmonary arterial hypertension:0.17), (CISAPRIDE:Pulmonary arterial hypertension:0.09), (CLIKSTAR:Pulmonary arterial hypertension:0.38), (CLOXAZOLAM:Pulmonary arterial hypertension:0.01), (COBICISTATELVITEGRAVIREMTRICITABINETENOFOVIR DISOPROXIL FUMARATE:Pulmonary arterial hypertension:0.46), (COLISTIN:Pulmonary arterial hypertension:0.27), (COLLAGEN:Pulmonary arterial hypertension:0.09), (CONVALLARIA MAJALISCRATAEGUS LAEVIGATA LEAFPROXYPHYLLINE:Pulmonary arterial hypertension:0.23), (CORTISONE ACETATE:Pulmonary arterial hypertension:0.5), (CRANBERRY JUICE:Pulmonary arterial hypertension:0.4), (DALFAMPRIDINE:Pulmonary arterial hypertension:0.55), (DAPAGLIFLOZIN PROPANEDIOLMETFORMIN HYDROCHLORIDE:Pulmonary arterial hypertension:0.09), (DEGARELIX:Pulmonary arterial hypertension:0.53), (DEXAMETHASONE ACETATE:Pulmonary arterial hypertension:1.27), (DEXTROAMPHETAMINE SULFATE:Pulmonary arterial hypertension:1.27), (DEXTROMETHORPHAN HYDROBROMIDEQUINIDINE SULFATE:Pulmonary arterial hypertension:0.09), (DIAZOXIDE:Pulmonary arterial hypertension:0.28), (DILAZEP:Pulmonary arterial hypertension:0.8), (DIPHENIDOL HYDROCHLORIDE:Pulmonary arterial hypertension:0.66), (DIPYRONE:Pulmonary arterial hypertension:0.8), (DIPYRONEPROMETHAZINE HYDROCHLORIDE:Pulmonary arterial hypertension:0.49), (DISTIGMINE BROMIDE:Pulmonary arterial hypertension:0.17), (DROSPIRENONEETHINYL ESTRADIOLLEVOMEFOLATE CALCIUM:Pulmonary arterial hypertension:0.07), (ECALLANTIDE:Pulmonary arterial hypertension:0.77), (EDARAVONE:Pulmonary arterial hypertension:1.12), (EFINACONAZOLE:Pulmonary arterial hypertension:0.5), (ELOTUZUMAB:Pulmonary arterial hypertension:0.56), (EMPAGLIFLOZINLINAGLIPTIN:Pulmonary arterial hypertension:0.75), (ENALAPRIL MALEATEHYDROCHLOROTHIAZIDE:Pulmonary arterial hypertension:0.36), (EPHEDRINE HYDROCHLORIDEGUAIFENESIN:Pulmonary arterial hypertension:0.43), (ESTRADIOL BENZOATE:Pulmonary arterial hypertension:0.33), (ESTRADIOLNORETHINDRONE ACETATE:Pulmonary arterial hypertension:0.46), (ETHINYL ESTRADIOLGESTODENE:Pulmonary arterial hypertension:0.48), (ETRETINATE:Pulmonary arterial hypertension:0.73), (EVENING PRIMROSE OIL:Pulmonary arterial hypertension:0.13), (EVOLOCUMAB:Pulmonary arterial hypertension:1.93), (FELBAMATE:Pulmonary arterial hypertension:0.07), (FENOTEROL HYDROBROMIDE:Pulmonary arterial hypertension:1.29), (FERRIC CITRATE ANHYDROUS:Pulmonary arterial hypertension:0.24), (FERRIC OXYHYDROXIDE:Pulmonary arterial hypertension:0.06), (FERRIC POLYSACCHARIDE COMPLEX:Pulmonary arterial hypertension:0.43), (FERROUS BISGLYCINATE:Pulmonary arterial hypertension:0.4), (FINGOLIMOD HYDROCHLORIDE:Pulmonary arterial hypertension:0.39), (FLAVIN ADENINE DINUCLEOTIDE:Pulmonary arterial hypertension:0.42), (FLURBIPROFEN:Pulmonary arterial hypertension:0.6), (FOSFOMYCIN:Pulmonary arterial hypertension:0.24), (FOSPHENYTOINFOSPHENYTOIN SODIUM:Pulmonary arterial hypertension:0.33), (FURSULTIAMINE:Pulmonary arterial hypertension:0.33), (GIMERACILOTERACILTEGAFUR:Pulmonary arterial hypertension:0.36), (GLIMEPIRIDEROSIGLITAZONE MALEATE:Pulmonary arterial hypertension:0.26), (GLYCOPYRROLATEINDACATEROL MALEATE:Pulmonary arterial hypertension:0.9), (GUANABENZ ACETATE:Pulmonary arterial hypertension:0.46), (HUMAN C1-ESTERASE INHIBITOR:Pulmonary arterial hypertension:0.12), (HYDROCORTISONE ACETATE:Pulmonary arterial hypertension:1.3), (HYDROXOCOBALAMIN:Pulmonary arterial hypertension:0.05), (ICOSAPENT:Pulmonary arterial hypertension:0.3), (IDARUCIZUMAB:Pulmonary arterial hypertension:0.53), (IMIGLUCERASE:Pulmonary arterial hypertension:0.35), (IMIQUIMOD:Pulmonary arterial hypertension:0.45), (INFLIXIMAB-DYYB:Pulmonary arterial hypertension:0.73), (INSULIN PORKINSULIN PURIFIED PORK:Pulmonary arterial hypertension:0.04), (INTERFERON:Pulmonary arterial hypertension:0.69), (IRONVITAMINS:Pulmonary arterial hypertension:0.55), (ITRACONAZOLE\SODIUM CHLORIDE:Pulmonary arterial hypertension:0.86), (IVACAFTOR:Pulmonary arterial hypertension:0.62), (IXAZOMIB:Pulmonary arterial hypertension:0.47), (LANREOTIDE:Pulmonary arterial hypertension:0.13), (LANREOTIDE ACETATE:Pulmonary arterial hypertension:0.22), (LAPATINIB DITOSYLATE:Pulmonary arterial hypertension:0.35), (LENOGRASTIM:Pulmonary arterial hypertension:0.35), (LEVOCARNITINE HYDROCHLORIDE:Pulmonary arterial hypertension:0.26), (LEVOTHYROXINE SODIUMPOTASSIUM IODIDE:Pulmonary arterial hypertension:0.01), (LULICONAZOLE:Pulmonary arterial hypertension:0.04), (MEPOLIZUMAB:Pulmonary arterial hypertension:1.63), (METFORMIN HYDROCHLORIDEROSIGLITAZONE MALEATE:Pulmonary arterial hypertension:0.61), (METHENAMINE MANDELATE:Pulmonary arterial hypertension:0.23), (METHENOLONE ENANTHATE:Pulmonary arterial hypertension:0.48), (MIGLUSTAT:Pulmonary arterial hypertension:0.13), (MORPHINE SULFATE\NALTREXONE HYDROCHLORIDE:Pulmonary arterial hypertension:0.19), (NALOXONE:Pulmonary arterial hypertension:0.04), (NIRAPARIB:Pulmonary arterial hypertension:0.38), (NITRENDIPINE:Pulmonary arterial hypertension:0), (OSPEMIFENE:Pulmonary arterial hypertension:0.66), (OXYBUTYNINOXYBUTYNIN CHLORIDE:Pulmonary arterial hypertension:0.73), (PALBOCICLIB:Pulmonary arterial hypertension:0.01), (PALIPERIDONE PALMITATE:Pulmonary arterial hypertension:0.26), (PARATHYROID HORMONE:Pulmonary arterial hypertension:0.22), (PEGINTERFERON BETA-1A:Pulmonary arterial hypertension:1.07), (PEGZEREPOETIN ALFA:Pulmonary arterial hypertension:0.22), (PENICILLIN V:Pulmonary arterial hypertension:0.55), (PERAMPANEL:Pulmonary arterial hypertension:0.06), (PHENYLEPHRINE HYDROCHLORIDEWITCH HAZEL:Pulmonary arterial hypertension:0.1), (PHLOROGLUCINOL1,3,5-TRIMETHOXYBENZENE:Pulmonary arterial hypertension:0.03), (POLYETHYLENE GLYCOL 3350POTASSIUM CHLORIDE\SODIUM BICARBONATE\SODIUM CHLORIDE\SODIUM SULFATE ANHYDROUS:Pulmonary arterial hypertension:0.42), (POLYSTYRENE:Pulmonary arterial hypertension:0.08), (POMALIDOMIDE:Pulmonary arterial hypertension:0.25), (POSACONAZOLE:Pulmonary arterial hypertension:0.49), (POVIDONE:Pulmonary arterial hypertension:0.57), (PREDNISOLONE SODIUM SUCCINATE:Pulmonary arterial hypertension:0.4), (PREGNENOLONE:Pulmonary arterial hypertension:0.13), (PROBUCOL:Pulmonary arterial hypertension:0.15), (PROPATYL NITRATE:Pulmonary arterial hypertension:0.03), (PROTRIPTYLINE HYDROCHLORIDE:Pulmonary arterial hypertension:1.21), (PRUCALOPRIDE SUCCINATE:Pulmonary arterial hypertension:0.37), (PYRAZINAMIDE:Pulmonary arterial hypertension:0.78), (RADIATION THERAPY:Pulmonary arterial hypertension:0.34), (RADIUM RA-223 DICHLORIDE:Pulmonary arterial hypertension:0.02), (RIFABUTIN:Pulmonary arterial hypertension:0.48), (RILMENIDINE PHOSPHATE:Pulmonary arterial hypertension:0.32), (ROFECOXIB:Pulmonary arterial hypertension:0.5), (SELEGILINE:Pulmonary arterial hypertension:0.34), (SERRAPEPTASE:Pulmonary arterial hypertension:0.2), (SIPULEUCEL-T:Pulmonary arterial hypertension:0.56), (SODIUM CITRATE:Pulmonary arterial hypertension:0.29), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Pulmonary arterial hypertension:0.07), (SODIUM PHOSPHATE, DIBASIC\SODIUM PHOSPHATE, MONOBASIC:Pulmonary arterial hypertension:1.03), (SOFOSBUVIRVELPATASVIR:Pulmonary arterial hypertension:0.06), (SUNITINIB MALATE:Pulmonary arterial hypertension:0.89), (SUPLATAST TOSILATE:Pulmonary arterial hypertension:0.33), (TAFAMIDIS MEGLUMINE:Pulmonary arterial hypertension:0.25), (TASIMELTEON:Pulmonary arterial hypertension:0.2), (TAZOBACTAM:Pulmonary arterial hypertension:0.46), (TEDUGLUTIDE\WATER:Pulmonary arterial hypertension:0.91), (TEMOCAPRIL HYDROCHLORIDE:Pulmonary arterial hypertension:0.35), (TESTOSTERONE UNDECANOATE:Pulmonary arterial hypertension:0.49), (THIOCTIC ACID:Pulmonary arterial hypertension:0.04), (TIMOLOL MALEATETRAVOPROST:Pulmonary arterial hypertension:0.88), (TIMOLOLTRAVOPROST:Pulmonary arterial hypertension:0.13), (TRIMIPRAMINE MALEATE:Pulmonary arterial hypertension:0.41), (TRIPTORELIN PAMOATE:Pulmonary arterial hypertension:0.12), (UBIQUINOL:Pulmonary arterial hypertension:0.03), (VALDECOXIB:Pulmonary arterial hypertension:1.02), (VORAPAXAR SULFATE:Pulmonary arterial hypertension:0.11), (ZALTOPROFEN:Pulmonary arterial hypertension:0.06), (ZICONOTIDE ACETATE:Pulmonary arterial hypertension:0.21), (.ALPHA.1-PROTEINASE INHIBITOR HUMAN:Pulmonary congestion:0.17), (ACETAMINOPHENBUTALBITALCAFFEINE:Pulmonary congestion:0.21), (AMIODARONE HYDROCHLORIDE:Pulmonary congestion:0.26), (AMITRIPTYLINE:Pulmonary congestion:0.41), (ANASTROZOLE:Pulmonary congestion:0.27), (CALCIUM ACETATE:Pulmonary congestion:0.2), (CALCIUM CARBONATECHOLECALCIFEROL:Pulmonary congestion:0.3), (CANDESARTAN CILEXETIL:Pulmonary congestion:0.37), (CARFILZOMIB:Pulmonary congestion:0.2), (CARVEDILOL PHOSPHATE:Pulmonary congestion:0.02), (CERTOLIZUMAB PEGOL:Pulmonary congestion:0.67), (CLOTRIMAZOLE:Pulmonary congestion:0.33), (COLCHICINE:Pulmonary congestion:0.29), (DACLIZUMAB:Pulmonary congestion:0.1), (DASATINIB:Pulmonary congestion:0.09), (DULOXETINE:Pulmonary congestion:0.21), (ELETRIPTAN HYDROBROMIDE:Pulmonary congestion:0.35), (ENALAPRIL MALEATE:Pulmonary congestion:0.16), (ENOXAPARIN:Pulmonary congestion:0.65), (ESZOPICLONE:Pulmonary congestion:0.2), (EVEROLIMUS:Pulmonary congestion:0.18), (FINGOLIMOD HYDROCHLORIDE:Pulmonary congestion:0.01), (FLECAINIDE:Pulmonary congestion:0.17), (FLUCONAZOLE:Pulmonary congestion:0.34), (FORMOTEROL FUMARATE:Pulmonary congestion:0.16), (FORMOTEROL FUMARATE\MOMETASONE FUROATE:Pulmonary congestion:0.44), (HYDROCHLOROTHIAZIDELISINOPRIL:Pulmonary congestion:0.23), (HYDROMORPHONE:Pulmonary congestion:0.1), (HYDROXYZINE PAMOATE:Pulmonary congestion:0.14), (HYOSCYAMINE:Pulmonary congestion:0.11), (ILOPROST:Pulmonary congestion:0.94), (INSULIN HUMAN:Pulmonary congestion:0.18), (NEBIVOLOL:Pulmonary congestion:0.18), (NILOTINIB:Pulmonary congestion:0.07), (OMEGA-3-ACID ETHYL ESTERS:Pulmonary congestion:0.09), (OXCARBAZEPINE:Pulmonary congestion:0.4), (PANTOPRAZOLE:Pulmonary congestion:1.1), (PEGINTERFERON BETA-1A:Pulmonary congestion:0.06), (PRAMIPEXOLE DIHYDROCHLORIDE:Pulmonary congestion:0.07), (PRIMIDONE:Pulmonary congestion:0.35), (SECUKINUMAB:Pulmonary congestion:0.12), (TOCILIZUMAB:Pulmonary congestion:0.08), (VARENICLINE TARTRATE:Pulmonary congestion:0.28), (VITAMIN B:Pulmonary congestion:0.35), (.ALPHA.1-PROTEINASE INHIBITOR HUMAN:Pulmonary embolism:1.01), (ABACAVIR SULFATEDOLUTEGRAVIR SODIUMLAMIVUDINE:Pulmonary embolism:0.36), (ABCIXIMAB:Pulmonary embolism:0.25), (ABOBOTULINUMTOXINA:Pulmonary embolism:0.29), (ACARBOSE:Pulmonary embolism:1.4), (ACETAMINOPHENDIPHENHYDRAMINE HYDROCHLORIDE:Pulmonary embolism:0.18), (ACTIVATED CHARCOAL:Pulmonary embolism:0.79), (AGALSIDASE BETA:Pulmonary embolism:1.56), (ALBIGLUTIDE:Pulmonary embolism:0.28), (ALBUMIN (HUMAN):Pulmonary embolism:0.17), (ALDESLEUKIN:Pulmonary embolism:0.86), (ALUMINUM HYDROXIDEMAGNESIUM OXIDEOXETHAZAINE:Pulmonary embolism:0.2), (AMINO ACIDSDEXTROSEELECTROLYTES NOS:Pulmonary embolism:1.14), (AMINOSALICYLIC ACID:Pulmonary embolism:0.13), (AMISULPRIDE:Pulmonary embolism:1.23), (AMOXICILLINCLARITHROMYCINESOMEPRAZOLE:Pulmonary embolism:0.33), (ANAGRELIDE HYDROCHLORIDE:Pulmonary embolism:0.56), (ANTIHISTAMINES NOS:Pulmonary embolism:0.64), (ARGININE:Pulmonary embolism:0.02), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Pulmonary embolism:0.16), (ASPIRINDIHYDROXYALUMINUM AMINOACETATE ANHYDROUSMAGNESIUM CARBONATE:Pulmonary embolism:0.59), (ATORVASTATIN CALCIUMEZETIMIBE:Pulmonary embolism:0.07), (AVANAFIL:Pulmonary embolism:0.01), (AZELNIDIPINE:Pulmonary embolism:0.39), (BACITRACIN:Pulmonary embolism:0.52), (BALSALAZIDE DISODIUM:Pulmonary embolism:0.26), (BELATACEPT:Pulmonary embolism:0.45), (BENZOCAINE:Pulmonary embolism:0.11), (BETAMETHASONE DIPROPIONATECALCIPOTRIENECALCIPOTRIENE HYDRATE:Pulmonary embolism:0.74), (BETAMETHASONE SODIUM PHOSPHATE:Pulmonary embolism:1.33), (BETAMETHASONECALCIPOTRIENE:Pulmonary embolism:0.64), (BETHANECHOL CHLORIDE:Pulmonary embolism:0.56), (BIPERIDEN HYDROCHLORIDE:Pulmonary embolism:0.27), (BLINATUMOMAB:Pulmonary embolism:1.39), (BOCOCIZUMAB:Pulmonary embolism:0.37), (BOSUTINIB:Pulmonary embolism:0.03), (CABAZITAXEL:Pulmonary embolism:0.49), (CALCIUM CARBONATECHOLECALCIFEROLMAGNESIUM CARBONATE:Pulmonary embolism:0.19), (CALCIUM CARBONATEERGOCALCIFEROLRETINOL:Pulmonary embolism:0.69), (CALCIUM CITRATEVITAMIN D:Pulmonary embolism:0.51), (CALCIUM POLYCARBOPHIL:Pulmonary embolism:0.62), (CALCIUM\MAGNESIUM:Pulmonary embolism:0.86), (CALCIUM\MAGNESIUM\ZINC:Pulmonary embolism:0.68), (CANGRELOR:Pulmonary embolism:0.05), (CAPSAICIN:Pulmonary embolism:0.24), (CARMUSTINE:Pulmonary embolism:0.11), (CARTEOLOL:Pulmonary embolism:0.05), (CEFEPIMECEFEPIME HYDROCHLORIDE:Pulmonary embolism:0.74), (CEVIMELINE HYDROCHLORIDE:Pulmonary embolism:0.03), (CHLOROTHIAZIDE:Pulmonary embolism:0.77), (CHONDROITIN SULFATE (CHICKEN):Pulmonary embolism:0.49), (CIPROFIBRATE:Pulmonary embolism:0.76), (CLOXACILLIN SODIUM:Pulmonary embolism:0.17), (COLISTIMETHATE SODIUM:Pulmonary embolism:0.33), (CYANOCOBALAMINFOLIC ACIDPYRIDOXINE:Pulmonary embolism:0.02), (CYPROHEPTADINE:Pulmonary embolism:1.41), (DACLATASVIR:Pulmonary embolism:0.49), (DACLIZUMAB:Pulmonary embolism:1.71), (DANTROLENE SODIUM:Pulmonary embolism:0.37), (DAPAGLIFLOZIN PROPANEDIOLMETFORMIN HYDROCHLORIDE:Pulmonary embolism:0.05), (DASABUVIROMBITASVIRPARITAPREVIRRITONAVIR:Pulmonary embolism:0.69), (DESVENLAFAXINE:Pulmonary embolism:0.06), (DESVENLAFAXINE SUCCINATE:Pulmonary embolism:1.13), (DEXAMETHASONE ACETATE:Pulmonary embolism:1.38), (DEXTROAMPHETAMINE SULFATE:Pulmonary embolism:0.84), (DIPHENHYDRAMINE HYDROCHLORIDELIDOCAINE HYDROCHLORIDENYSTATIN:Pulmonary embolism:0.19), (DIPYRIDAMOLE:Pulmonary embolism:1.79), (DISOPYRAMIDE:Pulmonary embolism:0.04), (DOLASETRON MESYLATE:Pulmonary embolism:0.83), (DULAGLUTIDE:Pulmonary embolism:0.46), (DUTASTERIDETAMSULOSIN HYDROCHLORIDE:Pulmonary embolism:0.98), (EDARAVONE:Pulmonary embolism:0.38), (ELBASVIRGRAZOPREVIR:Pulmonary embolism:0.46), (ELTROMBOPAG OLAMINE:Pulmonary embolism:0.47), (EMPAGLIFLOZINLINAGLIPTIN:Pulmonary embolism:0.9), (ERIBULIN:Pulmonary embolism:0.84), (ESOMEPRAZOLE SODIUM:Pulmonary embolism:0), (ESTROGENS, CONJUGATEDMEDROXYPROGESTERONE ACETATE:Pulmonary embolism:1.29), (ESTRONESUS SCROFA OVARY:Pulmonary embolism:0.83), (ETHACRYNIC ACID:Pulmonary embolism:0.14), (ETHINYL ESTRADIOLNORGESTIMATE:Pulmonary embolism:0.65), (ETHINYL ESTRADIOLNORGESTREL:Pulmonary embolism:0.33), (ETRETINATE:Pulmonary embolism:0.22), (FEXOFENADINEPSEUDOEPHEDRINE:Pulmonary embolism:0.74), (FIBRINOGEN HUMAN:Pulmonary embolism:0.11), (FLECAINIDE ACETATE:Pulmonary embolism:0.37), (FLUOROMETHOLONE:Pulmonary embolism:0.06), (FLUOROURACILLEUCOVORIN CALCIUMOXALIPLATIN:Pulmonary embolism:0.9), (FLURAZEPAMFLURAZEPAM HYDROCHLORIDE:Pulmonary embolism:0.49), (FLUTAMIDE:Pulmonary embolism:0.1), (FORMOTEROL FUMARATE\MOMETASONE FUROATE:Pulmonary embolism:1.02), (GENTAMICIN SULFATE:Pulmonary embolism:0.23), (GINGER:Pulmonary embolism:0.04), (GLUCAGON:Pulmonary embolism:0.42), (GLYCOSAMINOGLYCANS:Pulmonary embolism:0.85), (HYALURONATE SODIUM:Pulmonary embolism:0.54), (HYDROCHLOROTHIAZIDEMETOPROLOL SUCCINATE:Pulmonary embolism:0.29), (HYDROCORTISONE ACETATE:Pulmonary embolism:1.66), (IBRITUMOMAB TIUXETAN:Pulmonary embolism:0.67), (IMIGLUCERASE:Pulmonary embolism:0.61), (IMIPRAMINE HYDROCHLORIDE:Pulmonary embolism:1.67), (INFLIXIMAB-DYYB:Pulmonary embolism:1.11), (INOTUZUMAB OZOGAMICIN:Pulmonary embolism:0.65), (INTERFERON ALFA-2A:Pulmonary embolism:1.75), (INTERFERON ALFA-2B:Pulmonary embolism:1.35), (LACTOBACILLUS ACIDOPHILUSLACTOBACILLUS DELBRUECKII SUBSP. BULGARICUS:Pulmonary embolism:0.02), (LANREOTIDE:Pulmonary embolism:0.31), (LAPATINIB:Pulmonary embolism:0.19), (LAPATINIB DITOSYLATE:Pulmonary embolism:1.09), (LEVOMILNACIPRAN HYDROCHLORIDE:Pulmonary embolism:0), (LEVOSIMENDAN:Pulmonary embolism:0.08), (LEVOTHYROXINE SODIUMPOTASSIUM IODIDE:Pulmonary embolism:0.64), (LINSIDOMINE:Pulmonary embolism:0.56), (LIOTHYRONINE:Pulmonary embolism:0.6), (LOMUSTINE:Pulmonary embolism:0.32), (LUTETIUM LU-177:Pulmonary embolism:0.18), (MAGNESIUM ASPARTATE\POTASSIUM ASPARTATE:Pulmonary embolism:0.44), (MELPERONE:Pulmonary embolism:0.67), (MEPOLIZUMAB:Pulmonary embolism:0.95), (METFORMIN HYDROCHLORIDEROSIGLITAZONE MALEATE:Pulmonary embolism:0.08), (METFORMIN HYDROCHLORIDESAXAGLIPTIN HYDROCHLORIDE:Pulmonary embolism:0.3), (METHENOLONE ENANTHATE:Pulmonary embolism:0.06), (METHOXY POLYETHYLENE GLYCOL-EPOETIN BETA:Pulmonary embolism:0.18), (METHYLPREDNISOLONE ACETATE:Pulmonary embolism:2.15), (MITOXANTRONE HYDROCHLORIDE:Pulmonary embolism:0.85), (MOEXIPRIL HYDROCHLORIDE:Pulmonary embolism:0.51), (NILOTINIB:Pulmonary embolism:3.87), (OLOPATADINE:Pulmonary embolism:0.32), (OXYMETAZOLINE:Pulmonary embolism:0.42), (PALIPERIDONE PALMITATE:Pulmonary embolism:0.49), (PARATHYROID HORMONE:Pulmonary embolism:0.1), (PASIREOTIDE:Pulmonary embolism:0.21), (PATIROMER:Pulmonary embolism:0.67), (PEGINTERFERON BETA-1A:Pulmonary embolism:1.33), (PENICILLIN G:Pulmonary embolism:0.64), (PENICILLIN V POTASSIUM:Pulmonary embolism:0.16), (PENTOSAN POLYSULFATE SODIUM:Pulmonary embolism:0.98), (PENTOSTATIN:Pulmonary embolism:0.79), (PERAMPANEL:Pulmonary embolism:0.02), (PHENAZOPYRIDINE HYDROCHLORIDE:Pulmonary embolism:0.18), (PILSICAINIDE HYDROCHLORIDE:Pulmonary embolism:0.38), (PIMOBENDAN:Pulmonary embolism:0.4), (PIROXICAM:Pulmonary embolism:1.24), (PORFIMER SODIUM:Pulmonary embolism:0.26), (PRANLUKAST:Pulmonary embolism:0.04), (PROTRIPTYLINE HYDROCHLORIDE:Pulmonary embolism:0), (QUINACRINE:Pulmonary embolism:0.04), (ROSIGLITAZONE MALEATE:Pulmonary embolism:0.17), (SARPOGRELATE:Pulmonary embolism:0.26), (SEVELAMER HYDROCHLORIDE:Pulmonary embolism:0.67), (SODIUM CITRATE:Pulmonary embolism:0.3), (SODIUM PHOSPHATE, DIBASIC\SODIUM PHOSPHATE, MONOBASIC:Pulmonary embolism:0.49), (SOFOSBUVIRVELPATASVIR:Pulmonary embolism:0.38), (SPIRAMYCIN:Pulmonary embolism:0.14), (SUMATRIPTANSUMATRIPTAN SUCCINATE:Pulmonary embolism:1.94), (SUNITINIB:Pulmonary embolism:0.27), (TAFLUPROST:Pulmonary embolism:0.01), (TEDUGLUTIDE:Pulmonary embolism:0.17), (TELOTRISTAT ETHYL:Pulmonary embolism:0.63), (TESTOSTERONE ENANTHATE:Pulmonary embolism:0.04), (TETRAZEPAM:Pulmonary embolism:0.03), (THEOPHYLLINE:Pulmonary embolism:0.41), (THIOCTIC ACID:Pulmonary embolism:0.11), (TIBOLONE:Pulmonary embolism:0.02), (TICLOPIDINE:Pulmonary embolism:0.67), (TIGECYCLINE:Pulmonary embolism:0.57), (TIMOLOL MALEATETRAVOPROST:Pulmonary embolism:0.15), (TIROFIBAN HYDROCHLORIDE:Pulmonary embolism:0.21), (TREPROSTINIL DIOLAMINE:Pulmonary embolism:0.14), (TRIHEXYPHENIDYL:Pulmonary embolism:0.42), (TRIHEXYPHENIDYL HYDROCHLORIDE:Pulmonary embolism:0.02), (TRIPTORELIN PAMOATE:Pulmonary embolism:0.46), (VINORELBINE TARTRATE:Pulmonary embolism:1.27), (VOGLIBOSE:Pulmonary embolism:0.27), (ZICONOTIDE ACETATE:Pulmonary embolism:0.02), (ZILEUTON:Pulmonary embolism:0.47), (ACETAMINOPHENBUTALBITALCAFFEINE:Pulmonary fibrosis:0.12), (AMPHETAMINE ASPARTATEAMPHETAMINE SULFATEDEXTROAMPHETAMINE SACCHARATEDEXTROAMPHETAMINE SULFATE:Pulmonary fibrosis:0.13), (BISACODYL OR DOCUSATE SODIUM:Pulmonary fibrosis:0.5), (BRINZOLAMIDE:Pulmonary fibrosis:0.07), (BUSPIRONE HYDROCHLORIDE:Pulmonary fibrosis:0.46), (CALCIUM ACETATE:Pulmonary fibrosis:0.25), (CALCIUM CITRATE:Pulmonary fibrosis:0.04), (CANDESARTAN CILEXETILHYDROCHLOROTHIAZIDE:Pulmonary fibrosis:0.53), (CARVEDILOL PHOSPHATE:Pulmonary fibrosis:0.51), (CEFTRIAXONE SODIUM:Pulmonary fibrosis:0.04), (CHOLESTYRAMINE:Pulmonary fibrosis:0.08), (CLINDAMYCINCLINDAMYCIN PHOSPHATE:Pulmonary fibrosis:0.98), (DASATINIB:Pulmonary fibrosis:0.83), (DIVALPROEX SODIUM:Pulmonary fibrosis:0.65), (DOFETILIDE:Pulmonary fibrosis:0.17), (EDOXABAN TOSYLATE:Pulmonary fibrosis:0.11), (ENALAPRIL MALEATE:Pulmonary fibrosis:0.45), (EPINEPHRINE:Pulmonary fibrosis:1.07), (ESZOPICLONE:Pulmonary fibrosis:0.35), (EVEROLIMUS:Pulmonary fibrosis:0.37), (EVOLOCUMAB:Pulmonary fibrosis:1.17), (FEBUXOSTAT:Pulmonary fibrosis:0.11), (FINGOLIMOD HYDROCHLORIDE:Pulmonary fibrosis:0.35), (FLUTICASONE FUROATE:Pulmonary fibrosis:0.42), (FORMOTEROL:Pulmonary fibrosis:0.07), (FORMOTEROL FUMARATE\MOMETASONE FUROATE:Pulmonary fibrosis:1.09), (GLICLAZIDE:Pulmonary fibrosis:0.41), (HYDROCHLOROTHIAZIDEIRBESARTAN:Pulmonary fibrosis:0.45), (HYDROXYZINE PAMOATE:Pulmonary fibrosis:0.32), (IMATINIB MESYLATE:Pulmonary fibrosis:0.1), (INDAPAMIDE:Pulmonary fibrosis:0.33), (INDAPAMIDEPERINDOPRIL:Pulmonary fibrosis:0.43), (ISONIAZID:Pulmonary fibrosis:0.44), (LERCANIDIPINE HYDROCHLORIDE:Pulmonary fibrosis:0.25), (LITHIUM CARBONATE:Pulmonary fibrosis:0.16), (MEPOLIZUMAB:Pulmonary fibrosis:1.09), (METAXALONE:Pulmonary fibrosis:0.37), (METHENAMINE HIPPURATE:Pulmonary fibrosis:0.15), (METHYLPREDNISOLONE ACETATE:Pulmonary fibrosis:0.01), (NITROFURANTOIN:Pulmonary fibrosis:0.08), (OXCARBAZEPINE:Pulmonary fibrosis:0.67), (PEGINTERFERON BETA-1A:Pulmonary fibrosis:0.01), (PROPAFENONE HYDROCHLORIDE:Pulmonary fibrosis:0.22), (RABEPRAZOLE:Pulmonary fibrosis:0.41), (RALOXIFENE HYDROCHLORIDE:Pulmonary fibrosis:0.07), (RANOLAZINE:Pulmonary fibrosis:1.58), (SACUBITRILVALSARTAN:Pulmonary fibrosis:1.17), (SALMETEROL XINAFOATE:Pulmonary fibrosis:0.11), (SODIUM OXYBATE:Pulmonary fibrosis:0.18), (SOTALOL:Pulmonary fibrosis:0.78), (TERBUTALINE SULFATE:Pulmonary fibrosis:0.39), (TERIFLUNOMIDE:Pulmonary fibrosis:0.47), (TIMOLOL:Pulmonary fibrosis:0.32), (VALACYCLOVIR HYDROCHLORIDE:Pulmonary fibrosis:0.41), (ACETAMINOPHEN\CAFFEINE:Pulmonary hypertension:0.25), (ACETAMINOPHENDICHLORALPHENAZONEISOMETHEPTENE:Pul monary hypertension:0.09), (ACITRETIN:Pulmonary hypertension:0.4), (AGALSIDASE BETA:Pulmonary hypertension:1.1), (ALBIGLUTIDE:Pulmonary hypertension:1.01), (ALCOHOL:Pulmonary hypertension:0.79), (ALEMTUZUMAB:Pulmonary hypertension:0.25), (ALENDRONIC ACID:Pulmonary hypertension:1.18), (ALUMINUM HYDROXIDEMAGNESIUM HYDROXIDESIMETHICONE:Pulmonary hypertension:0.09), (AMIKACIN:Pulmonary hypertension:0.4), (AMISULPRIDE:Pulmonary hypertension:0.71), (AMLODIPINE BESYLATETELMISARTAN:Pulmonary hypertension:0.48), (ANTIHISTAMINES NOS:Pulmonary hypertension:0.35), (APREPITANT:Pulmonary hypertension:0.06), (ASCORBIC ACID/CHROMIUM/SELENIUM/VANADYL SULFATE:Pulmonary hypertension:0.1), (ASPIRINBUTALBITALCAFFEINE:Pulmonary hypertension:0.2), (ASUNAPREVIR:Pulmonary hypertension:0.36), (ATAZANAVIR:Pulmonary hypertension:0.23), (ATENOLOLCHLORTHALIDONE:Pulmonary hypertension:0.23), (AXITINIB:Pulmonary hypertension:0.52), (BAMIFYLLINE HYDROCHLORIDE:Pulmonary hypertension:0.02), (BETAMETHASONE DIPROPIONATECALCIPOTRIENECALCIPOTRIENE HYDRATE:Pulmonary hypertension:1.21), (BICARBONATE ION:Pulmonary hypertension:0.04), (BIFIDOBACTERIUM LONGUM SUBSP. INFANTIS:Pulmonary hypertension:1.09), (BOCOCIZUMAB:Pulmonary hypertension:0.26), (BRINZOLAMIDETIMOLOL MALEATE:Pulmonary hypertension:0.19), (CANAGLIFLOZINMETFORMIN HYDROCHLORIDE:Pulmonary hypertension:0.72), (CANAKINUMAB:Pulmonary hypertension:1.3), (CAPSAICIN:Pulmonary hypertension:0.42), (CEFEPIMECEFEPIME HYDROCHLORIDE:Pulmonary hypertension:0.47), (CEFPODOXIME PROXETIL:Pulmonary hypertension:0.28), (CEFPROZIL:Pulmonary hypertension:0.02), (CERIVASTATIN SODIUM:Pulmonary hypertension:0.1), (CLENBUTEROL HYDROCHLORIDE:Pulmonary hypertension:0.17), (CLIKSTAR:Pulmonary hypertension:0.26), (COLISTIMETHATE SODIUM:Pulmonary hypertension:0.26), (CORTISONE ACETATE:Pulmonary hypertension:0.41), (DACLIZUMAB:Pulmonary hypertension:1.12), (DANAZOL:Pulmonary hypertension:1.47), (DANTROLENE SODIUM:Pulmonary hypertension:0.01), (DAPAGLIFLOZIN PROPANEDIOL:Pulmonary hypertension:0.53), (DASABUVIROMBITASVIRPARITAPREVIRRITONAVIR:Pulmonary hypertension:0.21), (DASATINIB:Pulmonary hypertension:1.96), (DEGARELIX:Pulmonary hypertension:0.1), (DEXAMETHASONE ACETATE:Pulmonary hypertension:0.84), (DEXTROAMPHETAMINE:Pulmonary hypertension:0.13), (DEXTROAMPHETAMINE SULFATE:Pulmonary hypertension:0.92), (DIFLUNISAL:Pulmonary hypertension:0.28), (DIMETHICONESILICON DIOXIDESIMETHICONE:Pulmonary hypertension:0.49), (DIOSMIN:Pulmonary hypertension:0.09), (DIPYRONEPROMETHAZINE HYDROCHLORIDE:Pulmonary hypertension:0.2), (DISOPYRAMIDE:Pulmonary hypertension:0.04), (DOLASETRON MESYLATE:Pulmonary hypertension:0.1), (DOXYLAMINE SUCCINATE:Pulmonary hypertension:0.38), (ECALLANTIDE:Pulmonary hypertension:0.43), (EDARAVONE:Pulmonary hypertension:0.46), (EFINACONAZOLE:Pulmonary hypertension:0.11), (ELOTUZUMAB:Pulmonary hypertension:0.51), (EMPAGLIFLOZINLINAGLIPTIN:Pulmonary hypertension:0.27), (EPHEDRINE HYDROCHLORIDEGUAIFENESIN:Pulmonary hypertension:0.16), (ESOMEPRAZOLE MAGNESIUMNAPROXEN:Pulmonary hypertension:0.88), (ESTROGENS:Pulmonary hypertension:0.58), (ESTROGENS, CONJUGATEDMEDROXYPROGESTERONE ACETATE:Pulmonary hypertension:1.28), (ESTRONESUS SCROFA OVARY:Pulmonary hypertension:0.53), (ETRETINATE:Pulmonary hypertension:0.23), (FENOTEROL HYDROBROMIDE:Pulmonary hypertension:0.91), (FERROUS BISGLYCINATE:Pulmonary hypertension:0.11), (FERROUS SULFATEFOLIC ACID:Pulmonary hypertension:0.34), (FILGRASTIM:Pulmonary hypertension:0.51), (FLUVOXAMINE MALEATE:Pulmonary hypertension:1.06), (FUSIDIC ACID:Pulmonary hypertension:1.18), (GLUTAMINE:Pulmonary hypertension:0.01), (GLYCOPYRROLATE:Pulmonary hypertension:1.46), (GOLD:Pulmonary hypertension:0.17), (GUANFACINE:Pulmonary hypertension:0.61), (HUMAN C1-ESTERASE INHIBITOR:Pulmonary hypertension:0.32), (HYDROCHLOROTHIAZIDETELMISARTAN:Pulmonary hypertension:1.43), (HYOSCYAMINE SULFATEMETHENAMINEMETHYLENE BLUEPHENYL SALICYLATE\SODIUM PHOSPHATE, MONOBASIC, MONOHYDRATE:Pulmonary hypertension:0.08), (IDARUCIZUMAB:Pulmonary hypertension:0.48), (IMIPENEM:Pulmonary hypertension:0.08), (IMIPRAMINE HYDROCHLORIDE:Pulmonary hypertension:1.51), (INDAPAMIDEPERINDOPRIL:Pulmonary hypertension:1.68), (INFLIXIMAB-DYYB:Pulmonary hypertension:0.42), (INTERFERON:Pulmonary hypertension:0.67), (IRONVITAMINS:Pulmonary hypertension:0.09), (ITRACONAZOLE\SODIUM CHLORIDE:Pulmonary hypertension:0.19), (IVABRADINE HYDROCHLORIDE:Pulmonary hypertension:0.31), (LAFUTIDINE:Pulmonary hypertension:0), (MARAVIROC:Pulmonary hypertension:0.3), (MEPOLIZUMAB:Pulmonary hypertension:1.28), (METFORMIN HYDROCHLORIDEROSIGLITAZONE MALEATE:Pulmonary hypertension:0.88), (METHENAMINE HIPPURATE:Pulmonary hypertension:1.32), (METHENOLONE ENANTHATE:Pulmonary hypertension:0.34), (METHOXY POLYETHYLENE GLYCOL-EPOETIN BETA:Pulmonary hypertension:0.11), (MOEXIPRIL HYDROCHLORIDE:Pulmonary hypertension:0.35), (MORPHINE SULFATE\NALTREXONE HYDROCHLORIDE:Pulmonary hypertension:0.24), (NALTREXONE:Pulmonary hypertension:0.04), (NEFAZODONE:Pulmonary hypertension:0.09), (NEFAZODONE HYDROCHLORIDE:Pulmonary hypertension:0.05), (NEOMYCIN:Pulmonary hypertension:0.09), (NIRAPARIB:Pulmonary hypertension:0.1), (OSPEMIFENE:Pulmonary hypertension:0), (OXYBUTYNINOXYBUTYNIN CHLORIDE:Pulmonary hypertension:0.12), (PALIPERIDONE PALMITATE:Pulmonary hypertension:0.25), (PAMIDRONATE DISODIUM:Pulmonary hypertension:0.88), (PARATHYROID HORMONE:Pulmonary hypertension:0.12), (PATIROMER:Pulmonary hypertension:1.06), (PEGFILGRASTIM:Pulmonary hypertension:0.05), (PEGINTERFERON BETA-1A:Pulmonary hypertension:1.16), (PENICILLIN V:Pulmonary hypertension:0.43), (POLYETHYLENE GLYCOL 3350POTASSIUM CHLORIDE\SODIUM BICARBONATE\SODIUM CHLORIDE\SODIUM SULFATE ANHYDROUS:Pulmonary hypertension:0.15), (POLYETHYLENE GLYCOL 4000:Pulmonary hypertension:0.36), (POMALIDOMIDE:Pulmonary hypertension:0.29), (PONATINIB:Pulmonary hypertension:1.81), (POTASSIUM IODIDE:Pulmonary hypertension:0.1), (PRASTERONE:Pulmonary hypertension:0.1), (PROTRIPTYLINE HYDROCHLORIDE:Pulmonary hypertension:0.43), (PYRAZINAMIDE:Pulmonary hypertension:0.76), (QUINACRINE:Pulmonary hypertension:0.19), (RIVASTIGMINE:Pulmonary hypertension:0.97), (ROFECOXIB:Pulmonary hypertension:0.01), (SECUKINUMAB:Pulmonary hypertension:1.93), (SIBUTRAMINE HYDROCHLORIDE:Pulmonary hypertension:0.14), (SIPULEUCEL-T:Pulmonary hypertension:0.23), (SODIUM CITRATE:Pulmonary hypertension:0.08), (SORBITOL:Pulmonary hypertension:0.21), (TAFAMIDIS MEGLUMINE:Pulmonary hypertension:0.23), (TAZOBACTAM:Pulmonary hypertension:0.11), (TEDUGLUTIDE\WATER:Pulmonary hypertension:0.39), (TERBUTALINE:Pulmonary hypertension:1.12), (TESTOSTERONE CYPIONATE:Pulmonary hypertension:0.78), (TESTOSTERONE UNDECANOATE:Pulmonary hypertension:0.5), (THEOPHYLLINE:Pulmonary hypertension:0.55), (TIGECYCLINE:Pulmonary hypertension:0.04), (TRANEXAMIC ACID:Pulmonary hypertension:0.92), (TRIMIPRAMINE MALEATE:Pulmonary hypertension:0.13), (TURMERIC:Pulmonary hypertension:0.07), (YEAST:Pulmonary hypertension:0.35), (ISONIAZID:Pulmonary mycosis:0.37), (AMITRIPTYLINE:Pulmonary oedema:0.3), (ANASTROZOLE:Pulmonary oedema:0.34), (BENAZEPRIL HYDROCHLORIDE:Pulmonary oedema:0.02), (CANDESARTAN CILEXETILHYDROCHLOROTHIAZIDE:Pulmonary oedema:0.32), (CEFOTAXIME SODIUM:Pulmonary oedema:0.09), (CEFTRIAXONE SODIUM:Pulmonary oedema:0.39), (COLCHICINE:Pulmonary oedema:0.21), (CORTISONEHYDROCORTISONE:Pulmonary oedema:0.16), (DEVICE:Pulmonary oedema:0.15), (DEXLANSOPRAZOLE:Pulmonary oedema:0.38), (DICLOFENAC:Pulmonary oedema:0.41), (DOXAZOSINDOXAZOSIN MESYLATE:Pulmonary oedema:0.07), (FERROUS SULFATE:Pulmonary oedema:0.28), (FLUTICASONE:Pulmonary oedema:0.02), (FLUTICASONE FUROATEVILANTEROL TRIFENATATE:Pulmonary oedema:0.12), (HYDROCHLOROTHIAZIDELISINOPRIL:Pulmonary oedema:0.17), (HYDROMORPHONE HYDROCHLORIDE:Pulmonary oedema:0.04), (IBRUTINIB:Pulmonary oedema:1.42), (ILOPROST:Pulmonary oedema:0.1), (IMATINIB MESYLATE:Pulmonary oedema:0.49), (INDAPAMIDEPERINDOPRIL:Pulmonary oedema:0.02), (INFLUENZA VIRUS VACCINE:Pulmonary oedema:0.22), (IPRATROPIUM:Pulmonary oedema:0.08), (IRBESARTAN:Pulmonary oedema:0.13), (IRINOTECAN:Pulmonary oedema:0.05), (ITRACONAZOLE:Pulmonary oedema:0.05), (LERCANIDIPINE:Pulmonary oedema:0.6), (LERCANIDIPINE HYDROCHLORIDE:Pulmonary oedema:0.07), (LEVALBUTEROL HYDROCHLORIDE:Pulmonary oedema:0.03), (LOVASTATIN:Pulmonary oedema:0.24), (METHIMAZOLE:Pulmonary oedema:0.04), (MOXIFLOXACIN HYDROCHLORIDE:Pulmonary oedema:0.48), (NILOTINIB:Pulmonary oedema:0.85), (OMEPRAZOLE MAGNESIUM:Pulmonary oedema:0.44), (PANTOPRAZOLE MAGNESIUM:Pulmonary oedema:0.17), (PONATINIB:Pulmonary oedema:0.21), (RANOLAZINE:Pulmonary oedema:0.39), (SECUKINUMAB:Pulmonary oedema:0.1), (TOPIRAMATE:Pulmonary oedema:0.28), (VINCRISTINE SULFATE:Pulmonary oedema:0.17), (PYRAZINAMIDE:Pulmonary sarcoidosis:0.6), (AMIODARONE:Pulmonary thrombosis:0.37), (ANASTROZOLE:Pulmonary thrombosis:0.2), (BUMETANIDE:Pulmonary thrombosis:0.12), (DALFAMPRIDINE:Pulmonary thrombosis:0.02), (DILTIAZEM HYDROCHLORIDE:Pulmonary thrombosis:0.75), (DIPHENHYDRAMINE:Pulmonary thrombosis:0.26), (EVEROLIMUS:Pulmonary thrombosis:0.22), (EVOLOCUMAB:Pulmonary thrombosis:0.63), (IBRUTINIB:Pulmonary thrombosis:0.55), (ILOPROST:Pulmonary thrombosis:0.01), (INSULIN HUMAN:Pulmonary thrombosis:0.12), (PANTOPRAZOLE:Pulmonary thrombosis:0.68), (PHENOBARBITAL:Pulmonary thrombosis:0.1), (RAMIPRIL:Pulmonary thrombosis:0.25), (SACUBITRILVALSARTAN:Pulmonary thrombosis:0.07), (SODIUM OXYBATE:Pulmonary thrombosis:0.28), (TERIFLUNOMIDE:Pulmonary thrombosis:0.08), (TOCILIZUMAB:Pulmonary thrombosis:0.12), (TOPIRAMATE:Pulmonary thrombosis:0.72), (PEGINTERFERON ALFA-2A OR PEGINTERFERON ALFA-2B OR PEGINTERFERON BETA-1A OR PEGINTERFERON LAMBDA-1A:Pulmonary tuberculosis:0.11), (ANASTROZOLE:Pustular psoriasis:0.07), (BUDESONIDE:Pustular psoriasis:0.6), (CIPROFLOXACIN:Pustular psoriasis:0.05), (DEXLANSOPRAZOLE:Pustular psoriasis:0.24), (DICLOFENAC:Pustular psoriasis:0.59), (DIPHENHYDRAMINE:Pustular psoriasis:0.03), (DIVALPROEX SODIUM:Pustular psoriasis:0.16), (DOCUSATE SODIUM:Pustular psoriasis:0.21), (EVOLOCUMAB:Pustular psoriasis:0.68), (FERROUS SULFATEFERROUS SULFATE, DRIED:Pustular psoriasis:0.19), (HYDROXYCHLOROQUINE DIPHOSPHATE:Pustular psoriasis:0.01), (IBRUTINIB:Pustular psoriasis:0.2), (LOVASTATIN:Pustular psoriasis:0.15), (PANTOPRAZOLE:Pustular psoriasis:0.64), (PRAVASTATIN SODIUM:Pustular psoriasis:0.6), (SACUBITRILVALSARTAN:Pustular psoriasis:0.28), (WARFARIN:Pustular psoriasis:0.94), (AMIODARONE:Pyelonephritis:0.4), (AZITHROMYCIN ANHYDROUS:Pyelonephritis:0.09), (CALCIUM:Pyelonephritis:0.68), (CANDESARTAN:Pyelo nephritis:0.23), (CORTISONEHYDROCORTISONE:Pyelonephritis:0.23), (DALFAMPRIDINE: Pyelonephritis:0.19), (DESLORATADINE:Pyelonephritis:0.05), (DEXLANSOPRAZOLE:Pyelo nephritis:0.18), (DILTIAZEM:Pyelonephritis:0.55), (DIPHENHYDRAMINE:Pyelonephritis:0.15), (DOXAZOSINDOXAZOSIN MESYLATE:Pyelonephritis:0.12), (EVOLOCUMAB:Pyelonephritis:0.24), (FISH OIL:Pyelonephritis:0.21), (FLUTICASONE PROPIONATE:Pyelonephritis:0.4), (IBRUTINIB:Pyelonephritis:0.77), (IRON:Pyelonephritis:0.14), (LERCANIDIPINE HYDROCHLORIDE:Pyelonephritis:0.15), (LORATADINE:Pyelonephritis:0.02), (LOVASTATI N:Pyelonephritis:0.19), (NADROPARIN CALCIUM:Pyelonephritis:0.04), (NILOTINIB:Pyelonephritis:0.02), (OSELTAMIVIR:Pyeloneph ritis:0.18), (POLYETHYLENE GLYCOLS:Pyelonephritis:0.26), (POTASSIUM:Pyelonephritis:0.27), (PRAVASTATIN:Pyelone phritis:0.08), (RANITIDINE HYDROCHLORIDE:Pyelonephritis:0.07), (SACUBITRILVALSARTAN:Pyelonephritis:0.04), (TORSEMIDE:Pyelonephritis:0.14), (ZOLPIDEMZOLPIDEM TARTRATE:Pyelonephritis:0.19), (.ALPHA.-TOCOPHEROLRETINOL:Pyrexia:0.32), (.ALPHA.1-PROTEINASE INHIBITOR HUMAN:Pyrexia:2.43), (ABACAVIR SULFATEDOLUTEGRAVIR SODIUMLAMIVUDINE:Pyrexia:0.89), (ABOBOTULINUMTOXINA:Pyrexia:0.72), (ACAMP ROSATE:Pyrexia:0.06), (ACTIVATED CHARCOAL:Pyrexia:0.92), (ADENINE:Pyrexia:0.79), (ADENOSINE:Pyrexia:0.15), (AGALSID ASE BETA:Pyrexia:1.5), (ALBUTEROLGUAIFENESIN:Pyrexia:0.79), (ALECTINIB:Pyrexia:0.26), (ALISKIREN HEMIFUMARATE:Pyrexia:0.58), (ALTHIAZIDESPIRONOLACTONE:Pyrexia:0.46), (ALUM INUM SILICATE:Pyrexia:0.52), (AMEZINIUM METILSULFATE:Pyrexia:1.22), (AMINOPHYLLINE:Pyrexia:0.78), (AMLODIPINE BESYLATEHYDROCHLOROTHIAZIDEOLMESARTAN MEDOXOMIL:Pyrexia:0.07), (AMLODIPINE BESYLATEIRBESARTAN:Pyrexia:0.59), (AMMONIUM CHLORIDEDEXTROMETHORPHAN\SODIUM CITRATE:Pyrexia:0.23), (AMOXAPINE:Pyrexia:1.29), (AMOXICILLIN SODIUM:Pyrexia:0.41), (ARIPIPRAZOLE LAUROXIL:Pyrexia:0.21), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Pyrexia:0.08), (ASCORBIC ACIDVITAMIN B COMPLEX:Pyrexia:0.27), (ASIAN GINSENG:Pyrexia:0.22), (ATRACURIUM:Pyrexia:0.66), (AZELASTINE HYDROCHLORIDEFLUTICASONE PROPIONATE:Pyrexia:0.59), (AZELNIDIPINE:Pyrexia:1.4), (BAMIFYLLINE HYDROCHLORIDE:Pyrexia:0.38), (BELATACEPT:Pyrexia:0.98), (BENIDIPINE:Pyrexia:0.02), (BENPROPERINE PHOSPHATE:Pyrexia:0.19), (BERAPROST:Pyrexia:0.78), (BETAMETHASONE DIPROPIONATECALCIPOTRIENE:Pyrexia:0.76), (BETAMETHASONE DIPROPIONATECLOTRIMAZOLE:Pyrexia:0.9), (BETAMETHASONE SODIUM PHOSPHATENEOMYCIN SULFATE:Pyrexia:0.26), (BETAMETHASONECALCIPOTRIENE:Pyrexia:0.98), (BETAXOLOL HYDROCHLORIDE:Pyrexia:0.58), (BEXAROTENE:Pyrexia:0.06), (BLESELUMAB:Pyrexia:0.58), (BLONANSERIN:Pyrexia:0.19), (BOCOCIZUMAB:Pyrexia:0.29), (BOTULINUM TOXIN TYPE A:Pyrexia:0.81), (BOTULISM ANTITOXIN:Pyrexia:0.97), (BRINZOLAMIDE:Pyrexia:1.98), (CABOZANTINIB:Pyrexia:0.42), (CABOZANTINIB S-MALATE:Pyrexia:0.36), (CALCIUM CARBONATEERGOCALCIFEROL:Pyrexia:0.45), (CALCIUM CARBONATEERGOCALCIFEROLRETINOL:Pyrexia:1.47), (CALCIUM CHLORIDEDEXTROSE MONOHYDRATEMAGNESIUM CHLORIDE\SODIUM CHLORIDE\SODIUM LACTATE:Pyrexia:0.03), (CALCIUM CITRATEVITAMIN D:Pyrexia:0.25), (CALCIUM PANTOTHENATECYANOCOBALAMINNIACINAMIDEPYRIDOXINE HYDROCHLORIDE\RIBOFLAVINTHIAMINE MONONITRATE:Pyrexia:0.82), (CALCIUM PHOSPHATE:Pyrexia:0.05), (CALCIUM\MAGNESIUM\ZINC:Pyrexia:0.66), (CAPSAICIN:Pyr exia:0.65), (CARTEOLOL:Pyrexia:0.13), (CARTEOLOL HYDROCHLORIDE:Pyrexia:0.32), (CARVEDILOL PHOSPHATE:Pyrexia:1.95), (CEFTAROLINE:Pyrexia:0.42), (CHIMAPHILA UMBELLATAMANGANESE CHLORIDEPOPULUS TREMULOIDESSODIUM TAUROCHOLATE:Pyrexia:0.25), (CHLORMADINONE ACETATE:Pyrexia:0.51), (CHLORPROMAZINEPROMETHAZINE:Pyrexia:0.32), (CHOLINE BITARTRATECYSTEINEINOSITOLMAMMAL LIVER:Pyrexia:0.32), (CHONDROITIN SULFATE (CHICKEN):Pyrexia:1.42), (CICLOPIROX OLAMINE:Pyrexia:0.05), (CIFENLINE:Pyrexia:0.41), (CLENBUTEROL HYDROCHLORIDE:Pyrexia:0.62), (CLODRONATE DISODIUM:Pyrexia:0.53), (CLOFAZIMINE:Pyrexia:1.03), (CLOXAZOLAM:Pyrexia:0.35), (C OBICISTATELVITEGRAVIREMTRICITABINETENOFOVIR ALAFENAMIDE FUMARATE:Pyrexia:0.1), (COBICISTATELVITEGRAVIREMTRICITABINETENOFOVIR DISOPROXIL FUMARATE:Pyrexia:0.4), (COD LIVER OIL:Pyrexia:0.83), (COLESEVELAM HYDROCHLORIDE:Pyrexia:0.52), (COLESTILAN CHLORIDE:Pyrexia:0.04), (CROTALIDAE POLYVALENT IMMUNE FAB (OVINE):Pyrexia:0.62), (CYCLOPHOSPHAMIDECYTARABINEDEXAMETHASONEDOX ORUBICINMETHOTREXATEVINCRISTINE:Pyrexia:1.14), (CYCLOPHOSPHAMIDEDO XORUBICINPREDNISONERITUXIMABVINCRISTINE:Pyrexia:0.43), (CYPROTERONE ACETATE:Pyrexia:0.09), (DANAZOL:Pyrexia:2.33), (DAPAGLIFLOZIN:Pyrexia:0.18), (DAPA GLIFLOZIN PROPANEDIOLMETFORMIN HYDROCHLORIDE:Pyrexia:0.1), (DASABUVIROMBITASVIRPARITAPREVIRRITONAV IR:Pyrexia:0.45), (DEMECLOCYCLINE:Pyrexia:0), (DEOXYRIBONUCLIEC ACID:Pyrexia:0.59), (DESVENLAFAXINE:Pyrexia:0.18), (DESVENLAFAXINE SUCCINATE:Pyrexia:1.33), (DEXTRAN:Pyrexia:0.13), (DEXTROAMPHETAMINE SULFATE:Pyrexia:0.36), (DIAZOXIDE:Pyrexia:0.37), (DIFLUNISAL:Pyrexia:0.16), (DILAZEP: Pyrexia:1.06), (DIPYRONEPROMETHAZINE HYDROCHLORIDE:Pyrexia:0.74), (DISTIGMINE BROMIDE:Pyrexia:0.92), (ECABET:Pyrexia:0.22), (ECABET SODIUM:Pyrexia:0.63), (EFAVIRENZ:Pyrexia:0.01), (ELBASVIRGRAZOPREVIR:Pyrexia:0.09), (ELOSULFASE ALFA:Pyrexia:0.57), (EMPAGLIFLOZINLINAGLIPTIN:Pyrexia:0.94), (EMTRICITABINET ENOFOVIR DISOPROXIL:Pyrexia:0.16), (ENALAPRIL MALEATELERCANIDIPINE HYDROCHLORIDE:Pyrexia:0.37), (ENFUVIRTIDE:Pyrexia:0.13), (EPACADOSTAT:Pyrexia: 0.02), (EPHEDRA SINICA STEMHERBALS:Pyrexia:0.34), (EPHEDRINE:Pyrexia:0.9), (EPHEDRINE HYDROCHLORIDEGUAIFENESIN:Pyrexia:0.05), (ERIBULIN:Pyrexia:0.94), (ERYTHROM YCINSULFISOXAZOLE:Pyrexia:0.09), (ESTRONESUS SCROFA OVARY:Pyrexia:0.76), (ETHACRYNIC ACID:Pyrexia:0.52), (ETHINYL ESTRADIOLNORGESTREL:Pyrexia:0.51), (ETHYL LOFLAZEPATE:Pyrexia:0), (ETRETINATE:Pyrexia:2.52), (FELBAMATE:Pyrexia:0.46), (FEN OFIBRIC ACID:Pyrexia:0.65), (FENOTEROL HYDROBROMIDEIPRATROPIUM BROMIDE:Pyrexia:1.98), (FERRIC CITRATE ANHYDROUS:Pyrexia:0.1), (FERROUS SULFATEGLYCINE:Pyrexia:1.64), (FESOTERODINE:Pyrexia:0.03), (FIBRINOGEN HUMAN:Pyrexia:0.32), (FLAVIN ADENINE DINUCLEOTIDE:Pyrexia:0.88), (FLOMOXEF:Pyrexia:0.18), (FLUNARIZINE HYDROCHLORIDE:Pyrexia:0.22), (FLUOROURACILLEUCOVORIN CALCIUMOXALIPLATIN:Pyrexia:0.57), (FLUTICASONE PROPIONATEFORMOTEROL FUMARATE:Pyrexia:1.66), (FOLIC ACIDIRONMINERALSVITAMINS:Pyrexia:0.09), (FONDAPARINUX:Pyrexia:1.26), (FURS ULTIAMINE:Pyrexia:0.39), (FUSIDATE SODIUM:Pyrexia:0.4), (GADOLINIUM:Pyrexia:0.81), (GLIPIZIDEMETFORMIN HYDROCHLORIDE:Pyrexia:0.17), (GLYCOPYRROLATEINDACATEROL MALEATE:Pyrexia:1.17), (GOSERELIN ACETATE:Pyrexia:0.41), (GUAIFENESINPHENYLEPHRINE HYDROCHLORIDE:Pyrexia:0.17), (GUSPERIMUS TRIHYDROCHLORIDE:Pyrexia:0.66), (HYDROCHLOROTHIAZIDELOSARTAN:Pyrexia:0.38), (HYDROCHLOROTHIAZIDERAMIPRIL:Pyrexia:0.65), (HYDROCHLOROTHIAZIDES PIRONOLACTONE:Pyrexia:0.24), (HYDROCODONE BITARTRATEIBUPROFEN:Pyrexia:0.41), (HYDROCORTISONE PROBUTATE:Pyrexia:0.07), (HYDROXOCOBALAMIN:Pyrexia:0.15), (IBRITUMOMAB TIUXETAN:Pyrexia:2.69), (ICOSAPENT:Pyrexia:0.58), (IFENPRODIL:Pyrexia:0.19), (IGURA TIMOD:Pyrexia:1.06), (ILOPROST:Pyrexia:1.07), (IMIDAPRIL:Pyrexia:1.17), (IMIDAPRIL HYDROCHLORIDE:Pyrexia:0.03), (IMIPRAMINE:Pyrexia:0.78), (IMIPRAMINE HYDROCHLORIDE:Pyrexia:1.4), (INFLIXIMAB-DYYB:Pyrexia:0.69), (INSULIN PORKINSULIN PURIFIED PORK:Pyrexia:0.03), (INTERFERON ALFA-2A OR INTERFERON ALFA-2B:Pyrexia:0.57), (INTERFERON ALFA-2B:Pyrexia:1.42), (INTERFERON ALFA-N1:Pyrexia:0.23), (IODINE:Pyrexia:0.56), (IRONPOLYSACCHARIDES:Pyrexia:0.39), (ISOFL URANE:Pyrexia:0.79), (ISOSORBIDE:Pyrexia:2.03), (IXABEPILONE:Pyrexia:0.02), (LANREO TIDE:Pyrexia:0.48), (LAPATINIB DITOSYLATE:Pyrexia:0.78), (LENVATINIB:Pyrexia:0.73), (LEVOBUPIVACAINE:Pyrexia:0.52), (LEVOCARNITINE HYDROCHLORIDE:Pyrexia:1.45), (LEVOSIMENDAN:Pyrexia:0.23), (LEVOTHYROXINE SODIUMPOTASSIUM IODIDE:Pyrexia:1.62), (LINSIDOMINE:Pyrexia:0.35), (LOMITAPIDE MESYLATE:Pyrexia:0.47), (LUMACAFTOR:Pyrexia:1.09), (LUTETIUM LU-177:Pyrexia:0.14), (MAGNESIUM GLUCONATE:Pyrexia:0.63), (MAGNESIUM PIDOLATE:Pyrexia:0.31), (MANIDIPINE HYDROCHLORIDE:Pyrexia:0.04), (MAPROTILINE HYDROCHLORIDE:Pyrexia:0.25), (MELPERONE:Pyrexia:1.43), (MELPHALAN HYDROCHLORIDE:Pyrexia:0.1), (MEPENZOLATE BROMIDE:Pyrexia:0.06), (MEPREDNISONE:Pyrexia:0.15), (METFORMIN HYDROCHLORIDESAXAGLIPTIN:Pyrexia:0.14), (METFORMIN HYDROCHLORIDESAXAGLIPTIN HYDROCHLORIDE:Pyrexia:0.19), (METHENAMINE HIPPURATE:Pyrexia:2.14), (METHENOLONE ACETATE:Pyrexia:0.58), (METHOTREXATEMETHOTREXATE SODIUM:Pyrexia:0.41), (METHYLPREDNISOLONE OR METHYLPREDNISOLONE ACETATE:Pyrexia:0.55), (METRELEPTIN:Pyrexia:0.02), (MEXILETINE HYDROCHLORIDE:Pyrexia:0.74), (MITIGLINIDE CALCIUM HYDRATEVOGLIBOSE:Pyrexia:0.16), (MIZORIBINE:Pyrexia:1.71), (MOEXIPRIL HYDROCHLORIDE:Pyrexia:0.83), (MOGAMULIZUMAB:Pyrexia:1.3), (MORPHINEMORPH INE HYDROCHLORIDE\MORPHINE SULFATE\MORPHINE TARTRATE:Pyrexia:0.24), (MOSAPRIDE CITRATE DIHYDRATE:Pyrexia:0.74), (NABIXIMOLS:PPyrexia:0.69), (NAFAMOSTAT:Pyrexia:0.3), (NIL VADIPINE:Pyrexia:0), (NIRAPARIB:Pyrexia:1.19), (NITRENDIPINE:Pyrexia:0.32), (OK-432:Pyrexia:0.2), (OLODATEROL HYDROCHLORIDE:Pyrexia:0.32), (OMACETAXINE MEPESUCCINATE:Pyrexia:0.15), (OPIPRAMOL:Pyrexia:0.76), (OXATOMIDE:Pyrexia:0.33), (OXYBUTYNINOXYBUTYNIN CHLORIDE:Pyrexia:0.2), (OXYMORPHONE:Pyrexia:0.09), (PANIPENEM:Pyrexia:0.3), (PAN OBINOSTAT:Pyrexia:0.5), (PANTHENOL:Pyrexia:0.42), (PARICALCITOL:Pyrexia:0.81), (PE GINTERFERON LAMBDA-1A:Pyrexia:0.02), (PENICILLAMINE:Pyrexia:0.06), (PENICILLINV BENZATHINE:Pyrexia:0.58), (PENTOBARBITAL:Pyrexia:0.06), (PENTOSTATIN:Pyrexia:2.84), (PEROSPIRONE HYDROCHLORIDE:Pyrexia:0.08), (PHENAZOPYRIDINE HYDROCHLORIDE:Pyrexia:0.19), (PHENOTHIAZINE:Pyrexia:0.36), (PILSICAINIDE HYDROCHLORIDE:Pyrexia:1.35), (PIMOBENDAN:Pyrexia:0.96), (PINDOLOL:Pyrexia:0.03), (PITOFENONE:Pyrexia:0.04), (POLYCARBOPHIL:Pyrexia:0.14), (POLYMYXIN:Pyrexia:0.07), (POLYSTYRENE SULFONIC ACID:Pyrexia:0.77), (POTASSIUM CHLORIDE\SODIUM CHLORIDE\SODIUM LACTATE:Pyrexia:0.24), (POTASSIUM GLUCONATE:Pyrexia:0.05), (POVIDONE:Pyrexia:0.83), (PRAMOXINE HYDROCHLORIDE:Pyrexia:0.18), (PRAZOSIN HYDROCHLORIDE:Pyrexia:0.88), (PREDNISOLONE SODIUM METAZOATE:Pyrexia:0.21), (PREDNISONE ACETATE:Pyrexia:0.54), (PYRIDOSTIGMINE:Pyrexia:0.18), (PYRIDOXAL PHOSPHATE:Pyrexia:0.92), (PYRIMETHAMINE:Pyrexia:0.69), (PYRIMETHAMINESULFA DOXINE:Pyrexia:0.28), (QUINACRINE:Pyrexia:0.73), (QUINAPRIL:Pyrexia:0.95), (RASAGILINE MESYLATE:Pyrexia:0.41), (RETINOL:Pyrexia:1.46), (RHUBARB:Pyrexia:0.14), (RISEDRONI C ACID:Pyrexia:0.18), (RUFINAMIDE:Pyrexia:0.19), (RUPATADINE FUMARATE:Pyrexia:0.3), (SARPOGRELATE HYDROCHLORIDE:Pyrexia:0.84), (SCOPOLAMINE HYDROBROMIDE:Pyrexia:0.35), (SELEGILINE:Pyrexia:0.57), (SENNOSIDE B:Pyrexia:0.65), (SERRAPEPTASE:Pyrexia:0.71), (SIPULEUCEL-T:Pyrexia:0.43), (SOBUZOXANE:Pyrexia:0.24), (SOFOSBUVIRVELPATASVIR:Pyrexia:0.44), (SOMATOSTATIN:Pyrexia:0.35), (SUFENTANIL:Pyrexia:1.62), (SUGAMMADEX SODIUM:Pyrexia:0.11), (SULFAMERAZINETRIMETHOPRIM:Pyrexia:1.34), (SULINDAC:P yrexia:0.61), (SUPLATAST TOSILATE:Pyrexia:1.61), (TAFLUPROST:Pyrexia:0.37), (TAMOXIFEN CITRATE:Pyrexia:0.38), (TANDOSPIRONE:Pyrexia:0.31), (TAZOBACTAM SODIUM:Pyrexia:0.73), (TECELEUKIN:Pyrexia:0.32), (TEDUGLUTIDE\WATER:Pyrexia:1.5), (TELOTRISTAT ETHYL:Pyrexia:0.42), (TEMOCAPRIL HYDROCHLORIDE:Pyrexia:0.81), (TENELIGLIPTIN:Pyrexia:0.84), (THEOPHYLLINE:Pyrex ia:1.17), (THIAMYLAL:Pyrexia:0.81), (THIOGUANINE ANHYDROUS:Pyrexia:0.12), (THROMBOPOIETIN:Pyrexia:0.4), (TILIDINE:Pyrexia:0.27), (TI QUIZIUM BROMIDE:Pyrexia:0), (TISAGENLECLEUCEL:Pyrexia:1.18), (TRANILAST:Pyrexia:0.19), (T REPROSTINIL SODIUM:Pyrexia:0.2), (TRIMIPRAMINE MALEATE:Pyrexia:1.32), (TROPATEPINE:Pyrexia:0.22), (TULOBUTEROL HYDROCHLORIDE:Pyrexia:0.43), (UBENIMEX:Pyrexia:0.03), (UROKINASE:Pyrexia:0.97), (VANDETANIB:Pyrexia:0.09), (VINORELBINEVINORELBINE TARTRATE:Pyrexia:0.89), (XIPAMIDE:Pyrexia:0.1), (YEAST:Pyrexia:0.81), (ZAFIRLUKAST: Pyrexia:1.36), (ZALTOPROFEN:Pyrexia:0.67), (ZILEUTON:Pyrexia:1.08), (ZINC ACETATE:Pyrexia:0.22), (ZIV-AFLIBERCEPT:Pyrexia:0.76), (ACETAMINOPHENCODEINE PHOSPHATE:Radiculopathy:0.04), (BUDESONIDE:Radiculopathy:0.08), (CALCIUM CARBONATECHOLECALCIFEROL:Radiculopathy:0.2), (DALFAMPRIDINE:Radiculopathy: 0.28), (DIPHENHYDRAMINE:Radiculopathy:0.03), (EVOLOCUMAB:Radiculopathy:0.55), (F ERROUS SULFATEFERROUS SULFATE, DRIED:Radiculopathy:0.18), (IBRUTINIB:Radiculopathy:0.51), (ILOPROST:Radiculopathy:0.09), (NEBIVOLOL:Radiculopathy:0.1), (RABEPRAZOLE SODIUM:Radiculopathy:0.05), (RAMIPRIL:Radiculopathy:0.28), (RANOLAZINE:Radiculopath y:0.09), (SACUBITRILVALSARTAN:Radiculopathy:0.03), (SODIUM OXYBATE:Radiculopathy:0.18), (TERIFLUNOMIDE:Radiculopathy:0.46), (TOFACITINIB CITRATE:Radiculopathy:0.24), (VARENICLINE TARTRATE:Radiculopathy:0.23), (.ALPHA.1-PROTEINASE INHIBITOR HUMAN:Rash:1.91), (ABOBOTULINUMTOXINA:Rash:0.31), (ACETAMINOPHENCAFFEI NEOPIUM:Rash:0.2), (ACTIVATED CHARCOAL:Rash:0.55), (ADENINE:Rash:0.2), (AGALSIDASE BETA:Rash:1.16), (ALBIGLUTIDE:Rash:0.1), (ALFUZOSIN HYDROCHLORIDE:Rash:1.28), (ALISKIREN:Rash:0.27), (AMBROXOLCLENBUTEROL:Ra sh:0.48), (AMIKACIN SULFATE:Rash:0.85), (AMINO ACIDSCARBOHYDRATESELECTROLYTES NOSVITAMINS:Rash:0.13), (AMINOSALICYLIC ACID:Rash:0.4), (AMISULPRIDE:Rash:1.07), (AMLODIPINE BESYLATEIRBESARTAN:Rash:0.14), (AMLODIPINE BESYLATEOLMESARTAN MEDOXOMIL:Rash:1.04), (AMLODIPINEPERINDOPRIL:Rash:0.84), (AMMONIUM CHLORIDEDEXTROMETHORPHAN\SODIUM CITRATE:Rash:0.35), (AMOXAPINE:Rash:0.77), (AMPICILLIN SODIUM:Rash:0.13), (ANAGRELIDE HYDROCHLORIDE:Rash:0), (ANHYDROUS CITRIC ACIDASPIRIN\SODIUM BICARBONATE:Rash:0.1), (ARGININE:Rash:0.15), (ASCORBIC ACIDBIOTINCYANOCOBALAMINFOLIC ACIDNIACINPANTOTHENIC ACIDPYRIDOXINE\RIBOFLAVINTHIAMINE:Rash:0.08), (ASPIRINDIHYDROXYALUM INUM AMINOACETATE ANHYDROUSMAGNESIUM CARBONATE:Rash:0.02), (ASPIRINMAGNESIUM OXIDE:Rash:0.61), (BECLOMETHASONE DIPROPIONATEFORMOTEROL:Rash:0.15), (BECLOMETHASONEFORMOTEROL:Rash: 0.31), (BELATACEPT:Rash:0.44), (BENZALKONIUM CHLORIDE:Rash:0.17), (BEPRIDIL:Rash:0.28), (BERAPROST SODIUM:Rash:0.93), (BETAHISTINE MESILATE:Rash:0.43), (BETAMETHASONE DIPROPIONATECALCIPOTRIENE HYDRATE:Rash:0.41), (BETHANECHOL CHLORIDE:Rash:0.35), (BICARBONATE ION:Rash:0.24), (BLINATUMOMAB:Rash:1.36), (BOTULINUM TOXIN TYPE A:Rash:0.13), (BRIVARACETAM:Rash:0.08), (BUCILLAMINE:Rash:0.87), (BUDESONIDEF ORMOTEROL FUMARATE:Rash:1.43), (BUPIVACAINE HYDROCHLORIDE:Rash:0.24), (BUTALBITAL:Rash:0.23), (CABAZITAXEL:Rash:0.47), (CA BOZANTINIB S-MALATE:Rash:0.71), (CALCIUM CARBONATEERGOCALCIFEROLRETINOL:Rash:0.71), (CANAGLIFLOZINMETFORMIN HYDROCHLORIDE:Rash:1.9), (CARBIDOPA:Rash:0.31), (CARBIMAZOLE:Rash:0.98), (CAR MUSTINE:Rash:0.03), (CEFOTAXIMECEFOTAXIME SODIUM:Rash:0.59), (CEPHARANTHINE:Rash:0.27), (CERTOPARIN SODIUM:Rash:0.01), (CHLOROTHIAZIDE:Rash:0.31), (CINACALCET HYDROCHLORIDE:Rash:0.74), (CIPROFIBRATE:Rash:0.78), (CLODRONATE DISODIUM:Rash:0), (CLOFAZIMINE:Rash:0.05), (CLOMIPRAMINE HYDROCHLORIDE:Rash:0.57), (COBICISTATELVITEGRAVIREMTRICITABINETENOF OVIR DISOPROXIL FUMARATE:Rash:0.31), (COLISTIMETHATE SODIUM:Rash:0.86), (CORTICOTROPIN:Rash:0.99), (CYAMEMAZINE:Rash:0.1), (DACLAT ASVIR:Rash:0.42), (DANAZOL:Rash:1.09), (DANTROLENE SODIUM:Rash:0.56), (DEXMEDETOMIDINE HYDROCHLORIDE:Rash:0.24), (DEXTROAMPHETAMINE:Rash:0.03), (DIGITOXIN:Rash:00.59), (DIPYRONEPROMETHAZINE HYDROCHLORIDE:Rash:0.08), (DOBUTAMINE HYDROCHLORIDE:Rash:0.07), (DOLASETRON MESYLATE:Rash:1.15), (DOPAMINE HYDROCHLORIDE:Rash:0.44), (EDOXABAN TOSILATE MONOHYDRATE:Rash:0.07), (EFAVIRENZEMTRICITABINETENOFOVIR DISOPROXIL FUMARATE:Rash:0.7), (ELBASVIRGRAZOPREVIR:Rash:0.03), (ELOTUZUMAB:Rash:1.63), (EMPAGLIFLOZINLINAGLIPTIN:Rash:1.14), (EMTRICITABINE:Rash:0.33), (EMTRICIT ABINERILPIVIRINE HYDROCHLORIDETENOFOVIR DISOPROXIL FUMARATE:Rash:0.22), (ENALAPRIL MALEATELERCANIDIPINE HYDROCHLORIDE:Rash:0.14), (ERIBULIN:Rash:0.77), (ERIBULIN MESYLATE:Rash:0.48), (ERTAPENEM SODIUM:Rash:0.4), (ESTROGENS:Rash:0.48), (EUCALYPTOLMENTHOLMETHYL SALICYLATETHYMOL:Rash:0.27), (FELBAMATE:Rash:0.27), (FENOTEROL HYDROBROMIDEIPRATROPIUM BROMIDE:Rash:0.8), (FERROUS SULFATEFOLIC ACID:Rash:0.23), (FERROUS SULFATEGLYCINE:Rash:0.21), (FLAVIN ADENINE DINUCLEOTIDE:Rash:0.82), (FLUCYTOSINE:Rash:0.38), (FLUOROURACILLEUCOVORI N CALCIUMOXALIPLATIN:Rash:0.49), (FLURAZEPAMFLURAZEPAM HYDROCHLORIDE:Rash:0.14), (FLUTAMIDE:Rash:0.01), (FOLIC ACIDIRONMINERALSVITAMINS:Rash:0.05), (FONDAPARINUX:Rash:0.1), (FORMOTE ROL FUMARATE\MOMETASONE FUROATE:Rash:1.49), (GLIPIZIDEMETFORMIN HYDROCHLORIDE:Rash:0.28), (GLUTAMINE:Rash:0.31), (GLYCOPYRROLATEINDACA TEROL MALEATE:Rash:0.49), (GLYCOPYRRONIUMINDACATEROL:Rash:1.93), (GUANFACINE: Rash:0.48), (HYALURONIC ACID:Rash:0.14), (HYDROCODONE BITARTRATEIBUPROFEN:Rash:1.02), (IBRITUMOMAB TIUXETAN:Rash:0.78), (IDARUBICIN:Rash:0.13), (IGURATIMOD:Rash:0.22), (IMIGLUCER ASE:Rash:0.52), (IMIPENEM:Rash:0.87), (IMIPRAMINE:Rash:0.18), (IMMUNE GLOBULIN NOS:Rash:0.39), (INDAPAMIDEPERINDOPRIL:Rash:2.09), (INFLIXIMAB-DYYB:Rash:0.68), (INTERFERON ALFA-2A:Rash:1.79), (INTERFERON ALFA-2A OR INTERFERON ALFA-2B:Rash:0.3), (INTERFERON GAMMA-1B:Rash:1.12), (IRONPOLYSACCHARIDES:Rash:0.17), (ACIDOPHILUSLACTOBACILLUS DELBRUECKII SUBSP. BULGARICUS:Rash:0.15), (LANREOTIDE:Rash:0.12), (LECITHIN:Rash:0.15), (LEVOMEFO LATE CALCIUM:Rash:0.53), (LEVOTHYROXINE SODIUMPOTASSIUM IODIDE:Rash:0.1), (LEVOTHYROXINELIOTHYRONINE:Rash:0.23), (LISDEXAMFETAMINE DIMESYLATE:Rash:1.21), (LOMUSTINE:Rash:0.09), (LUMACAFTOR:Rash:0.2), (LURASID ONE HYDROCHLORIDE:Rash:0.22), (LYSOZYME HYDROCHLORIDE:Rash:0.39), (MAGNESIUM ASPARTATE\POTASSIUM ASPARTATE:Rash:0.06), (METFORMIN HYDROCHLORIDESAXAGLIPTIN:Rash:0.05), (METFORMIN HYDROCHLORIDEVILDAGLIPTIN:Rash:0.83), (METFORMIN PAMOATE:Rash:0.15), (METHENOLONE ENANTHATE:Rash:1.09), (MEXILETINE HYDROCHLORIDE:Rash:0.06), (MITIGLINIDE:Rash:0.25), (MOXONIDINE:Rash:1.05), (NA DROPARIN CALCIUM:Rash:1.46), (NALTREXONE:Rash:0.27), (OCTREOTIDE:Rash:0.32), (ORPHENAD RINE:Rash:0.56), (OSELTAMIVIR:Rash:1.3), (OXYBUTYNINOXYBUTYNIN CHLORIDE:Rash:0.35), (PALIPERIDONE PALMITATE:Rash:0.02), (PENTOSTATIN:Rash:1.17), (PHENAZOPYRIDINE HYDROCHLORIDE:Rash:0.33), (PHENOBARBITAL SODIUM:Rash:0.29), (PIMOBENDAN:Rash:0.26), (PODOFILOX:Rash:0.3), (POLAPRE\ZINC: Rash:0.27), (PORFIMER SODIUM:Rash:0.46), (POTASSIUM CITRATETRISODIUM CITRATE DIHYDRATE:Rash:0.79), (PRAMOXINE HYDROCHLORIDE:Rash:0.41), (PRASTERONE:Rash:0.3), (PROPAFENONE:Rash:0.51), (PR OTRIPTYLINE HYDROCHLORIDE:Rash:0.12), (PSORALEN:Rash:0.19), (PYRIMETHAMINE:Rash:0.37), (Q UINACRINE:Rash:0.52), (RILMENIDINE PHOSPHATE:Rash:0.25), (RIOCIGUAT:Rash:0.57), (RIVASTIGMINE:Rash:1.32), (ROFLUMI LAST:Rash:2.15), (RUCAPARIB CAMSYLATE:Rash:0.25), (SARPOGRELATE HYDROCHLORIDE:Rash:0.11), (SODIUM CITRATE:Rash:0.11), (SOFOSBUVIRVELPATASVIR:Rash:0.34), (SOTALOL HYDROCHLORIDE:Rash:0.2), (SUFENTANIL CITRATE:Rash:0.55), (TAFLUPROST:Rash:0.32), (TAMOXIFEN CITRATE:Rash:0.3), (TBO-FILGRASTIM:Rash:0.06), (TEDUGLUTIDE:Rash:0.17), (TELOTRISTAT ETHYL:Rash:0.56), (TEMSIROLIMUS:Rash:0.49), (TESTOSTERONE UNDECANOATE:Rash:0.36), (THEOPHYLLINE:Rash:0.6), (THIOPENTAL SODIUM:Rash:0.07), (THROMBOMODULIN ALFA:Rash:0.05), (TOFACITINIB:Rash:0.34), (TRIHEXYPHENIDYL:Rash:0.07), (TRIMIPRA MINE MALEATE:Rash:0.34), (TRIPTORELIN PAMOATE:Rash:0.07), (UMECLIDINIUM BROMIDE:Rash:1.07), (UMECLIDINIUM BROMIDEVILANTEROL TRIFENATATE:Rash:1.8), (VARDENAFIL HYDROCHLORIDE TRIHYDRATE:Rash:0.38), (VINORELBINE TARTRATE:Rash:1.29), (VINORELBINEVINORELBINE TARTRATE:Rash:0.47), (VONOPRAZAN:Rash:0.11), (YEAST:Rash:0.58), (ZAFIRLUKAST:R ash:1.01), (ZICONOTIDE ACETATE:Rash:0.05), (ZILEUTON:Rash:0.79), (ZIV-AFLIBERCEPT:Rash:0.46), (BISOPROLOL FUMARATE:Rash pruritic:0.05), (BUPROPION HYDROCHLORIDE:Rash pruritic:0.04), (DEXLANSOPRAZOLE:Rash pruritic:0.26), (ENALAPRIL:Rash pruritic:0.47), (EVOLOCUMAB:Rash pruritic:0.08), (FERROUS SULFATE:Rash pruritic:0.08), (IBRUTINIB:Rash pruritic:1.16), (ILOPROST:Rash pruritic:0.01), (LOVASTATIN:Rash pruritic:0.2), (METOPROLOL TARTRATE:Rash pruritic:0.02), (PANTOPRAZOLE:Rash pruritic:0.33), (POLYETHYLENE GLYCOL 3350:Rash pruritic:0.08), (SACUBITRILVALSARTAN:Rash pruritic:0.11), (TOFACITINIB CITRATE:Rash pruritic:0.18), (AMIODARONE:Raynaud's phenomenon:0.55), (BISACODYL OR DOCUSATE SODIUM:Raynaud's phenomenon:0.36), (BORTEZOMIB:Raynaud's phenomenon:0.09), (BUDESONIDEFORMOTEROL FUMARATE:Raynaud's phenomenon:0.02), (BUPRENORPHINE HYDROCHLORIDENALOXONE HYDROCHLORIDE:Raynaud's phenomenon:0.18), (CARVEDILOL PHOSPHATE:Raynaud's phenomenon:0.2), (CHOLESTYRAMINE:Raynaud's phenomenon:0.86), (CICLESONIDE:Raynaud's phenomenon:0.21), (CLINDAMYCINCLINDAMYCIN PHOSPHATE:Raynaud's phenomenon:0.53), (CLOTRIMAZOLE:Raynaud's phenomenon:0.24), (CORTISONEHYDROCORTISONE:Raynaud's phenomenon:0.62), (DACLIZUMAB:Raynaud's phenomenon:0.52), (DASATINIB:Raynaud's phenomenon:0.24), (DEVICE:Raynaud's phenomenon:0.19), (DIETARY SUPPLEMENT:Raynaud's phenomenon:0.17), (DIMENHYDRINATE:Raynaud's phenomenon:0.16), (DOCUSATE CALCIUM:Raynaud's phenomenon:0.07), (DOXAZOSINDOXAZOSIN MESYLATE:Raynaud's phenomenon:0.22), (DUTASTERIDE:Raynaud's phenomenon:0.41), (ELETRIPTAN HYDROBROMIDE:Raynaud's phenomenon:0.76), (ENALAPRIL:Raynaud's phenomenon:1.51), (ENOXAPARIN:Raynaud's phenomenon:0.34), (ESZOPICLONE:Raynaud's phenomenon:0.85), (EVOLOCUMAB:Raynaud's phenomenon:1.07), (FLUTICASONE FUROATEVILANTEROL TRIFENATATE:Raynaud's phenomenon:0.57), (GLYBURIDE:Raynaud's phenomenon:0.11), (HYDRALAZINE HYDROCHLORIDE:Raynaud's phenomenon:0.16), (HYDROCHLOROTHIAZIDELOSARTAN POTASSIUM:Raynaud's phenomenon:0.08), (HYDROCHLOROTHIAZIDEVALSARTAN:Raynaud's phenomenon:0.02), (HYDROMORPHONE:Raynaud's phenomenon:0.17), (HYDROXYZINE PAMOATE:Raynaud's phenomenon:0.68), (IMATINIB MESYLATE:Raynaud's phenomenon:0.21), (IMIPRAMINE HYDROCHLORIDE:Raynaud's phenomenon:0.25), (INDAPAMIDE:Raynaud's phenomenon:0.09), (ISOSORBIDE:Raynaud's phenomenon:0.33), (LEDIPASVIRSOFOSBUVIR:Raynaud's phenomenon:0.05), (MOXIFLOXACIN HYDROCHLORIDE:Raynaud's phenomenon:0.43), (NITROFURANTOIN:Raynaud's phenomenon:0.33), (OLANZAPINE:Raynaud's phenomenon:0.09), (OXCARBAZEPINE:Raynaud's phenomenon:0.31), (PANTOPRAZOLE MAGNESIUM:Raynaud's phenomenon:0.72), (PAROXETINEPAROXETINE HYDROCHLORIDE:Raynaud's phenomenon:0.11), (PEGFILGRASTIM:Raynaud's phenomenon:0.1), (RABEPRAZOLE:Raynaud's phenomenon:0.12), (RANOLAZINE:Raynaud's phenomenon:0.76), (RIOCIGUAT:Raynaud's phenomenon:0.85), (SACUBITRILVALSARTAN:Raynaud's phenomenon:0.27), (SCOPOLAMINE:Raynaud's phenomenon:0.03), (SULFAMETHOXAZOLE:Raynaud's phenomenon:0.28), (TAMSULOSIN:Raynaud's phenomenon:1.16), (UMECLIDINIUM BROMIDEVILANTEROL TRIFENATATE:Raynaud's phenomenon:0.56), (VILAZODONE HYDROCHLORIDE:Raynaud's phenomenon:0.2), (VITAMIN B:Raynaud's phenomenon:0.81), (ACENOCOUMAROL:Rectal cancer:0.08), (ACETAMINOPHENBUTALBITALCAFFEINE:Rectal cancer:0.33), (ACETAZOLAMIDE:Rectal cancer:0.04), (AMIODARONE HYDROCHLORIDE:Rectal cancer:0.82), (ANASTROZOLE:Rectal cancer:1.5), (AXITINIB:Rectal cancer:0.23), (BENAZEPRILBENAZEPRIL HYDROCHLORIDE:Rectal cancer:0.09), (CALCITONIN SALMON:Rectal cancer:0.53), (CALCITRIOL:Rectal cancer:0.84), (CALCIUM ACETATE:Rectal cancer:0.01), (CARFILZOMIB:Rectal cancer:0.48), (CARVEDILOL PHOSPHATE:Rectal cancer:0.36), (CERITINIB:Rectal cancer:0.2), (CERTOLIZUMAB PEGOL:Rectal cancer:0.8), (CHLORTHALIDONE:Rectal cancer:0.07), (CLOTRIMAZOLE:Rectal cancer:0.58), (CLOZAPINE:Rectal cancer:0.79), (DABRAFENIB:Rectal cancer:0.69), (DALTEPARIN:Rectal cancer:0.17), (DASATINIB:Rectal cancer:0.94), (DESLORATADINE:Rectal cancer:0.3), (DEVICE:Rectal cancer:0.27), (DEXAMETHASONE ACETATE:Rectal cancer:0.12), (EPINASTINE HYDROCHLORIDE:Rectal cancer:0.28), (ERIBULIN:Rectal cancer:0.2), (ESTROGENS, CONJUGATED:Rectal cancer:0.02), (EVOLOCUMAB:Rectal cancer:0.56), (EXEMESTANE:Rectal cancer:0.69), (EZETIMIBESIMVASTATIN:Rectal cancer:0.14), (FLECAINIDE:Rectal cancer:0.63), (FLUOXETINEFLUOXETINE HYDROCHLORIDE:Rectal cancer:0.24), (FLUTICASONE FUROATE:Rectal cancer:0.14), (FLUTICASONE PROPIONATE:Rectal cancer:1.26), (FORMOTEROL FUMARATE:Rectal cancer:0), (GEFITINIB:Rectal cancer:0.23), (HYALURONATE SODIUM:Rectal cancer:0.01), (HYDROCHLOROTHIAZIDEIRBESARTAN:Rectal cancer:0.47), (HYDROCHLOROTHIAZIDETELMISARTAN:Rectal cancer:0), (HYDROCHLOROTHIAZIDEVALSARTAN:Rectal cancer:0.52), (HYDROXYUREA:Rectal cancer:0.39), (IBRUTINIB:Rectal cancer:0.86), (INDAPAMIDEPERINDOPRIL:Rectal cancer:0.4), (INFLUENZA VIRUS VACCINE:Rectal cancer:0.22), (INVESTIGATIONAL PRODUCT:Rectal cancer:0.45), (LABETALOLLABETALOL HYDROCHLORIDE:Rectal cancer:0.13), (LERCANIDIPINE HYDROCHLORIDE:Rectal cancer:0.24), (LETROZOLE:Rectal cancer:0.72), (LEVALBUTEROL HYDROCHLORIDE:Rectal cancer:0.37), (LITHIUM CARBONATE:Rectal cancer:0.51), (MECLIZINEMECLIZINE HYDROCHLORIDE:Rectal cancer:0.15), (METAXALONE:Rectal cancer:0.11), (METOLAZONE:Rectal cancer:0.09), (MIDODRINE:Rectal cancer:0.04), (NADOLOL:Rectal cancer:0), (NEBIVOLOL:Rectal cancer:0.25), (NILOTINIB:Rectal cancer:1.68), (NIRAPARIB:Rectal cancer:0.24), (OMEGA-3-ACID ETHYL ESTERS:Rectal cancer:0.73), (OSIMERTINIB:Rectal cancer:0.16), (PALBOCICLIB:Rectal cancer:0.01), (PANTOPRAZOLE MAGNESIUM:Rectal cancer:0.64), (PEMETREXED:Rectal cancer:0.75), (PEMETREXED DISODIUM:Rectal cancer:0.35), (PONATINIB:Rectal cancer:0.09), (PRAMIPEXOLE DIHYDROCHLORIDE:Rectal cancer:0.52), (PRIMIDONE:Rectal cancer:0.73), (ROFLUMILAST:Rectal cancer:0.56), (SACUBITRILVALSARTAN:Rectal cancer:0.12), (SECUKINUMAB:Rectal cancer:0.26), (SULFAMETHOXAZOLE:Rectal cancer:0.41), (TETRACYCLINE:Rectal cancer:0.07), (THALIDOMIDE:Rectal cancer:0.66), (TIOTROPIUM:Rectal cancer:0.09), (TIZANIDINE:Rectal cancer:0.26), (TOCILIZUMAB:Rectal cancer:0.3), (TRIAMCINOLONE ACETONIDE:Rectal cancer:0.29), (VALACYCLOVIR HYDROCHLORIDE:Rectal cancer:0.03), (VARENICLINE TARTRATE:Rectal cancer:0.07), (VINORELBINE TARTRATE:Rectal cancer:0.19), (VITAMIN B:Rectal cancer:0.51), (UNSPECIFIED INGREDIENT:Rectosigmoid cancer:0.12), (PRAVASTATIN SODIUM:Red blood cell count decreased:0.15), (RAMIPRIL:Red blood cell count decreased:0.22), (.ALPHA.1-PROTEINASE INHIBITOR HUMAN:Reflux gastritis:0.1), (ACARBOSE:Reflux gastritis:0.07), (ALCOHOL:Reflux gastritis:0.16), (AMISULPRIDE:Reflux gastritis:0.03), (ANASTROZOLE:Reflux gastritis:1.56), (AZITHROMYCIN DIHYDRATE:Reflux gastritis:0.24), (BENAZEPRIL HYDROCHLORIDE:Reflux gastritis:0.68), (BISACODYL OR DOCUSATE SODIUM:Reflux gastritis:0.69), (BUPRENORPHINE HYDROCHLORIDENALOXONE HYDROCHLORIDE:Reflux gastritis:0.27), (CALCIUM ACETATE:Reflux gastritis:0.68), (CANDESARTAN CILEXETILHYDROCHLOROTHIAZIDE:Reflux gastritis:0.91), (CARVEDILOL PHOSPHATE:Reflux gastritis:0.14), (CODEINE:Reflux gastritis:0.18), (DACLIZUMAB:Reflux gastritis:0.06), (DALFAMPRIDINE:Reflux gastritis:0.32), (DAPAGLIFLOZIN PROPANEDIOL:Reflux gastritis:0.01), (DASATINIB:Reflux gastritis:1.19), (DEVICE:Reflux gastritis:0.19), (DIMENHYDRINATE:Reflux gastritis:0.09), (DIPHENHYDRAMINE:Reflux gastritis:1.17), (DIVALPROEX SODIUM:Reflux gastritis:0.63), (DOCETAXEL:Reflux gastritis:0.1), (DRONABINOL:Reflux gastritis:0.17), (EDOXABAN TOSYLATE:Reflux gastritis:0.2), (ELETRIPTAN HYDROBROMIDE:Reflux gastritis:0.43), (ETORICOXIB:Reflux gastritis:0.04), (FEBUXOSTAT:Reflux gastritis:0.09), (FLUTICASONE:Reflux gastritis:0.56), (FORMOTEROL FUMARATE\MOMETASONE FUROATE:Reflux gastritis:0.21), (HYDRALAZINE HYDROCHLORIDE:Reflux gastritis:0.46), (HYDROXYUREA:Reflux gastritis:0.46), (HYDROXYZINE PAMOATE:Reflux gastritis:0.51), (ILOPROST:Reflux gastritis:0.07), (INDAPAMIDEPERINDOPRIL:Reflux gastritis:0.45), (INFLUENZA VIRUS VACCINE:Reflux gastritis:0.52), (LEDIPASVIRSOFOSBUVIR:Reflux gastritis:0.73), (LERCANIDIPINE:Reflux gastritis:0.44), (LERCANIDIPINE HYDROCHLORIDE:Reflux gastritis:0.58), (MEGESTROL ACETATE:Reflux gastritis:0.11), (METAXALONE:Reflux gastritis:0.4), (METHYLPREDNISOLONE ACETATE:Reflux gastritis:0.17), (METRONIDAZOLE:Reflux gastritis:1.64), (MIDODRINE:Reflux gastritis:0.13), (MOXIFLOXACIN HYDROCHLORIDE:Reflux gastritis:0.88), (NADOLOL:Reflux gastritis:0.13), (NORTRIPTYLINE:Reflux gastritis:0.26), (PITAVASTATIN CALCIUM:Reflux gastritis:0.01), (PYRIDOXINE HYDROCHLORIDE:Reflux gastritis:0.09), (RALOXIFENE HYDROCHLORIDE:Reflux gastritis:0.01), (RIZATRIPTAN BENZOATE:Reflux gastritis:0.14), (SCOPOLAMINE:Reflux gastritis:0.2), (SULFAMETHOXAZOLE:Reflux gastritis:0.08), (SUMATRIPTANSUMATRIPTAN SUCCINATE:Reflux gastritis:0.38), (TERIFLUNOMIDE:Reflux gastritis:0.12), (TOLTERODINE TARTRATE:Reflux gastritis:0.15), (TRIAMTERENE:Reflux gastritis:0.03), (VARENICLINE TARTRATE:Reflux gastritis:0.83), (VITAMIN B:Reflux gastritis:0.74), (ZINC:Reflux gastritis:0.07), (.ALPHA.1-PROTEINASE INHIBITOR HUMAN:Relapsing-remitting multiple sclerosis:0.01), (ACETAMINOPHENOXYCODONE HYDROCHLORIDEOXYCODONE TEREPHTHALATE:Relapsing-remitting multiple sclerosis:0.42), (ACETAMINOPHENPROPOXYPHENE HYDROCHLORIDE:Relapsing-remitting multiple sclerosis:0.72), (ACETAMINOPHENPROPOXYPHENE NAPSYLATE:Relapsing-remitting multiple sclerosis:0.19), (AGALSIDASE BETA:Relapsing-remitting multiple sclerosis:0.19), (ALCOHOL:Relapsing-remitting multiple sclerosis:0.13), (ALENDRONIC ACID:Relapsing-remitting multiple sclerosis:0.26), (ALISKIREN HEMIFUMARATE:Relapsing-remitting multiple sclerosis:0.39), (AMLODIPINE BESYLATEATORVASTATIN CALCIUM:Relapsing-remitting multiple sclerosis:0.21), (AMLODIPINEVALSARTAN:Relapsing-remitting multiple sclerosis:0.34), (ASPIRINDIHYDROXYALUMINUM AMINOACETATE ANHYDROUSMAGNESIUM CARBONATE:Relapsing-remitting multiple sclerosis:0.12), (BALSALAZIDE DISODIUM:Relapsing-remitting multiple sclerosis:0.14), (BENZTROPINE:Relapsing-remitting multiple sclerosis:0.28), (BETAMETHASONECALCIPOTRIENE:Relapsing-remitting multiple sclerosis:0.13), (BIFIDOBACTERIUM LONGUM SUBSP. INFANTIS:Relapsing-remitting multiple sclerosis:0.45), (BILASTINE:Relapsing-remitting multiple sclerosis:0.1), (BRIMONIDINE TARTRATETIMOLOL MALEATE:Relapsing-remitting multiple sclerosis:0.1), (BUPIVACAINE HYDROCHLORIDE:Relapsing-remitting multiple sclerosis:0.11), (BUPRENORPHINE HYDROCHLORIDE:Relapsing-remitting multiple sclerosis:0.88), (CALCIPOTRIENE:Relapsing-remitting multiple sclerosis:0.24), (CALCITONIN SALMON:Relapsing-remitting multiple sclerosis:0.47), (CALCIUM ACETATE:Relapsing-remitting multiple sclerosis:0.31), (CALCIUM CITRATEVITAMIN D:Relapsing-remitting multiple sclerosis:0.13), (CANAGLIFLOZIN:Relapsing-remitting multiple sclerosis:0.17), (CANAGLIFLOZINMETFORMIN HYDROCHLORIDE:Relapsing-remitting multiple sclerosis:0.14), (CANAKINUMAB:Relapsing-remitting multiple sclerosis:0.18), (CANDESARTAN CILEXETILHYDROCHLOROTHIAZIDE:Relapsing-remitting multiple sclerosis:1.22), (CARBIDOPA:Relapsing-remitting multiple sclerosis:0.07), (CARFILZOMIB:Relapsing-remitting multiple sclerosis:0.33), (CERTOLIZUMAB PEGOL:Relapsing-remitting multiple sclerosis:1.67), (CEVIMELINE HYDROCHLORIDE:Relapsing-remitting multiple sclerosis:0.08), (CHLORDIAZEPOXIDE HYDROCHLORIDECLIDINIUM BROMIDE:Relapsing-remitting multiple sclerosis:0.46), (CLINDAMYCIN HYDROCHLORIDE:Relapsing-remitting multiple sclerosis:0.19), (CYPROHEPTADINE:Relapsing-remitting multiple sclerosis:0.33), (DABIGATRAN:Relapsing-remitting multiple sclerosis:0.35), (DAPAGLIFLOZIN PROPANEDIOL:Relapsing-remitting multiple sclerosis:0.32), (DASATINIB:Relapsing-remitting multiple sclerosis:0.46), (DIPYRIDAMOLE:Relapsing-remitting multiple sclerosis:0.44), (DOFETILIDE:Relapsing-remitting multiple sclerosis:0.44), (DUTASTERIDETAMSULOSIN HYDROCHLORIDE:Relapsing-remitting multiple sclerosis:0.06), (ECONAZOLE:Relapsing-remitting multiple sclerosis:0.49), (EDOXABAN TOSYLATE:Relapsing-remitting multiple sclerosis:0.5), (EMPAGLIFLOZINLINAGLIPTIN:Relapsing-remitting multiple sclerosis:0.36), (EPLERENONE:Relapsing-remitting multiple sclerosis:0.36), (ERYTHROPOIETIN:Relapsing-remitting multiple sclerosis:0.34), (EVEROLIMUS:Relapsing-remitting multiple sclerosis:0.3), (EVOLOCUMAB:Relapsing-remitting multiple sclerosis:1.17), (FERROUS BISGLYCINATE:Relapsing-remitting multiple sclerosis:0.07), (FERROUS SULFATEGLYCINE:Relapsing-remitting multiple sclerosis:0.39), (FISH OIL\OMEGA-3 FATTY ACIDS:Relapsing-remitting multiple sclerosis:0.32), (FLUDROCORTISONE ACETATE:Relapsing-remitting multiple sclerosis:0.82), (FLUNITRAZEPAM:Relapsing-remitting multiple sclerosis:0.04), (FLURAZEPAMFLURAZEPAM HYDROCHLORIDE:Relapsing-remitting multiple sclerosis:0), (FLUVOXAMINE MALEATE:Relapsing-remitting multiple sclerosis:0.66), (FORMOTEROL FUMARATE:Relapsing-remitting multiple sclerosis:0.75), (FORMOTEROL FUMARATE DIHYDRATE\MOMETASONE FUROATE:Relapsing-remitting multiple sclerosis:0.67), (FORMOTEROL FUMARATE\MOMETASONE FUROATE:Relapsing-remitting multiple sclerosis:0.22), (FUSIDIC ACID:Relapsing-remitting multiple sclerosis:0.63), (GLYCOPYRROLATE:Relapsing-remitting multiple sclerosis:0.35), (GLYCOPYRRONIUMINDACATEROL:Relapsing-remitting multiple sclerosis:0.52), (HEPARIN CALCIUM:Relapsing-remitting multiple sclerosis:0.29), (HYALURONATE SODIUM:Relapsing-remitting multiple sclerosis:0.29), (HYDROXYCHLOROQUINE DIPHOSPHATE:Relapsing-remitting multiple sclerosis:0.71), (IBRUTINIB:Relapsing-remitting multiple sclerosis:0.15), (IMATINIB MESYLATE:Relapsing-remitting multiple sclerosis:0.18), (INDAPAMIDEPERINDOPRIL:Relapsing-remitting multiple sclerosis:0.65), (INFLIXIMAB-DYYB:Relapsing-remitting multiple sclerosis:0.65), (INSULIN BEEF:Relapsing-remitting multiple sclerosis:0.43), (INSULIN GLULISINE:Relapsing-remitting multiple sclerosis:0.29), (INVESTIGATIONAL PRODUCT:Relapsing-remitting multiple sclerosis:0.03), (IPRATROPIUM:Relapsing-remitting multiple sclerosis:1.19), (ISOPROPYL ALCOHOLLEUPROLIDE ACETATENORETHINDRONE ACETATE:Relapsing-remitting multiple sclerosis:0.08), (LACTOBACILLUS RHAMNOSUS:Relapsing-remitting multiple sclerosis:0.17), (LEDIPASVIRSOFOSBUVIR:Relapsing-remitting multiple sclerosis:0.01), (LEVALBUTEROL HYDROCHLORIDE:Relapsing-remitting multiple sclerosis:1.09), (LEVOMEFOLATE CALCIUM:Relapsing-remitting multiple sclerosis:0.44), (LOMITAPIDE MESYLATE:Relapsing-remitting multiple sclerosis:0.23), (METHYLPREDNISOLONE ACETATE:Relapsing-remitting multiple sclerosis:2.11), (MIDODRINE HYDROCHLORIDE:Relapsing-remitting multiple sclerosis:0.19), (MOMETASONE:Relapsing-remitting multiple sclerosis:0.03), (MOXIFLOXACIN:Relapsing-remitting multiple sclerosis:0.2), (MUPIROCIN:Relapsing-remitting multiple sclerosis:0.62), (NALOXONE HYDROCHLORIDEOXYCODONE HYDROCHLORIDE:Relapsing-remitting multiple sclerosis:1.25), (NATEGLINIDE:Relapsing-remitting multiple sclerosis:0.25), (NICARDIPINE:Relapsing-remitting multiple sclerosis:0.4), (NILOTINIB:Relapsing-remitting multiple sclerosis:0.59), (OSPEMIFENE:Relapsing-remitting multiple sclerosis:0.33), (PHYTONADIONE:Relapsing-remitting multiple sclerosis:0.03), (PILOCARPINE:Relapsing-remitting multiple sclerosis:0.2), (PIOGLITAZONE:Relapsing-remitting multiple sclerosis:0.11), (PIROXICAM:Relapsing-remitting multiple sclerosis:0.62), (PROPAFENONE HYDROCHLORIDE:Relapsing-remitting multiple sclerosis:0.26), (PROTRIPTYLINE HYDROCHLORIDE:Relapsing-remitting multiple sclerosis:0.13), (PYRAZINAMIDE:Relapsing-remitting multiple sclerosis:0.64), (RIVASTIGMINE:Relapsing-remitting multiple sclerosis:0.41), (SECUKINUMAB:Relapsing-remitting multiple sclerosis:0.59), (SULINDAC:Relapsing-remitting multiple sclerosis:0.28), (TERBUTALINE:Relapsing-remitting multiple sclerosis:0.19), (TERBUTALINE SULFATE:Relapsing-remitting multiple sclerosis:0.87), (TESTOSTERONE UNDECANOATE:Relapsing-remitting multiple sclerosis:0.44), (THYROID, PORCINETHYROID, UNSPECIFIED:Relapsing-remitting multiple sclerosis:0.46), (TIOTROPIUM:Relapsing-remitting multiple sclerosis:0.39), (TOCILIZUMAB:Relapsing-remitting multiple sclerosis:0.74), (TOFACITINIB CITRATE:Relapsing-remitting multiple sclerosis:0.3), (TRANEXAMIC ACID:Relapsing-remitting multiple sclerosis:0.01), (TRAVOPROST:Relapsing-remitting multiple sclerosis:0.16), (UMECLIDINIUM BROMIDEVILANTEROL TRIFENATATE:Relapsing-remitting multiple sclerosis:0.5), (VALGANCICLOVIR HYDROCHLORIDE:Relapsing-remitting multiple sclerosis:0.1), (VANCOMYCIN HYDROCHLORIDE:Relapsing-remitting multiple sclerosis:0.29), (VORTIOXETINE HYDROBROMIDE:Relapsing-remitting multiple sclerosis:0.12), (ALCOHOL:Relaxation therapy:0.1), (ARMODAFINIL:Relaxation therapy:0.09), (CERTOLIZUMAB PEGOL:Relaxation therapy:0.11), (DEXLANSOPRAZOLE:Relaxation therapy:0.96), (DICLOFENAC:Relaxation therapy:0.1), (DIPHENHYDRAMINE:Relaxation therapy:0.03), (ESOMEPRAZOLE:Relaxation therapy:0.01), (EVEROLIMUS:Relaxation therapy:0.07), (EVOLOCUMAB:Relaxation therapy:0.13), (FERROUS SULFATE:Relaxation therapy:0.04), (FERROUS SULFATEFERROUS SULFATE, DRIED:Relaxation therapy:0.33), (HYDROXYCHLOROQUINE SULFATE:Relaxation therapy:0.12), (IBRUTINIB:Relaxation therapy:0.22), (LISDEXAMFETAMINE DIMESYLATE:Relaxation therapy:0.23), (LOVASTATIN:Relaxation therapy:0.41), (MAGNESIUM:Relaxation therapy:0.35), (PAROXETINE:Relaxation therapy:0.19), (POLYETHYLENE GLYCOLS:Relaxation therapy:0.1), (PRAMIPEXOLE:Relaxation therapy:0.21), (PRAVASTATIN:Relaxation therapy:0.03), (RAMIPRIL:Relaxation therapy:0.74), (SACUBITRILVALSARTAN:Relaxation therapy:0.02), (TERIFLUNOMIDE:Relaxation therapy:0.21), (UMECLIDINIUM BROMIDEVILANTEROL TRIFENATATE:Relaxation therapy:0.07), (VERAPAMIL HYDROCHLORIDE:Relaxation therapy:0.39), (ACARBOSE:Renal cancer:0.65), (ACETAMINOPHEN\CAFFEINE:Renal cancer:0.03), (ADO-TRASTUZUMAB EMTANSINE:Renal cancer:1.26), (AFATINIB:Renal cancer:0.17), (AGALSIDASE BETA:Renal cancer:0.15), (ALENDRONIC ACID:Renal cancer:0.46), (AMINO ACIDSDEXTROSEELECTROLYTES NOS:Renal cancer:0.06), (AMLODIPINE BESYLATEHYDROCHLOROTHIAZIDEOLMESARTAN MEDOXOMIL:Renal cancer:0.5), (AMLODIPINE BESYLATEHYDROCHLOROTHIAZIDEVALSARTAN:Renal cancer:0.46), (ARMODAFINIL:Renal cancer:0.82), (AZILSARTAN KAMEDOXOMIL:Renal cancer:0.12), (BECLOMETHASONE DIPROPIONATE:Renal cancer:0.44), (BETAMETHASONE DIPROPIONATECALCIPOTRIENECALCIPOTRIENE HYDRATE:Renal cancer:0.2), (BORTEZOMIB:Renal cancer:1.18), (BUDESONIDEFORMOTEROL FUMARATE:Renal cancer:0.31), (BUPRENORPHINE HYDROCHLORIDE:Renal cancer:0.55), (BUTYLSCOPOLAMINE BROMIDE:Renal cancer:0.35), (CALCITONIN SALMON:Renal cancer:0.71), (CALCIUM\MAGNESIUM:Renal cancer:0.25), (CANDESARTAN:Renal cancer:2.46), (CARFILZOMIB:Renal cancer:1.18), (CASANTHRANOLDOCUSATE SODIUM:Renal cancer:0.17), (CEFAZOLIN SODIUM:Renal cancer:0.02), (CEFOTAXIME SODIUM:Renal cancer:0.09), (CEFUROXIME AXETIL:Renal cancer:0.04), (CERTOLIZUMAB PEGOL:Renal cancer:1.18), (CETIRIZINE HYDROCHLORIDEPSEUDOEPHEDRINE HYDROCHLORIDE:Renal cancer:0.28), (CHLORDIAZEPOXIDE HYDROCHLORIDECLIDINIUM BROMIDE:Renal cancer:0.54), (CILASTATIN SODIUMIMIPENEM:Renal cancer:0.13), (CLOBAZAM:Renal cancer:0.02), (CLOZAPINE:Renal cancer:0.97), (COD LIVER OIL:Renal cancer:0.38), (CORTICOTROPIN:Renal cancer:0.44), (DABIGATRAN:Renal cancer:0.39), (DABRAFENIB:Renal cancer:0.44), (DACLIZUMAB:Renal cancer:0.31), (DALTEPARIN:Renal cancer:0.56), (DANAZOL:Renal cancer:0.1), (DARATUMUMAB:Renal cancer:0.03), (DASABUVIROMBITASVIRPARITAPREVIRRITONAVIR:Renal cancer:0.07), (DEFLAZACORT:Renal cancer:0.59), (DESVENLAFAXINE SUCCINATE:Renal cancer:0.61), (DEVICE:Renal cancer:0.55), (DEXAMETHASONE ACETATE:Renal cancer:0.2), (DIGITOXIN:Renal cancer:0.2), (DRONEDARONE:Renal cancer:0.42), (EDOXABAN TOSYLATE:Renal cancer:0.92), (ELETRIPTAN HYDROBROMIDE:Renal cancer:1.1), (ELOTUZUMAB:Renal cancer:0.14), (EMPAGLIFLOZINLINAGLIPTIN:Renal cancer:0.58), (ERIBULIN:Renal cancer:0.19), (ESOMEPRAZOLE MAGNESIUMNAPROXEN:Renal cancer:0.08), (ESTRONESUS SCROFA OVARY:Renal cancer:0.12), (EVOLOCUMAB:Renal cancer:1.17), (FLUTICASONE FUROATESALMETEROL XINAFOATE:Renal cancer:0.3), (FLUVOXAMINE MALEATE:Renal cancer:0.02), (FUSIDIC ACID:Renal cancer:0.42), (GIMERACILOTERACILTEGAFUR:Renal cancer:0.36), (GLYCOPYRROLATE:Renal cancer:0.04), (GLYCOPYRRONIUMINDACATEROL:Renal cancer:0.37), (HYALURONATE SODIUM:Renal cancer:0.17), (HYDROXYCHLOROQUINE DIPHOSPHATE:Renal cancer:0.07), (ILOPROST:Renal cancer:0.22), (IMIPRAMINE HYDROCHLORIDE:Renal cancer:0.24), (IXAZOMIB:Renal cancer:0.23), (LACTASE:Renal cancer:0.52), (LANREOTIDE ACETATE:Renal cancer:0.06), (LEDIPASVIRSOFOSBUVIR:Renal cancer:1.4), (LERCANIDIPINE HYDROCHLORIDE:Renal cancer:0.97), (LEVOLEUCOVORIN CALCIUM:Renal cancer:0.09), (LISDEXAMFETAMINE DIMESYLATE:Renal cancer:0.22), (LOMITAPIDE MESYLATE:Renal cancer:0.6), (MEFENAMIC ACID:Renal cancer:0.19), (METFORMIN HYDROCHLORIDEVILDAGLIPTIN:Renal cancer:0.48), (METHYLPREDNISOLONE ACETATE:Renal cancer:1.05), (MIDODRINE HYDROCHLORIDE:Renal cancer:0.15), (NABUMETONE:Renal cancer:0.66), (NALOXONE HYDROCHLORIDETILIDINE HYDROCHLORIDE:Renal cancer:0.32), (NATEGLINIDE:Renal cancer:0.69), (NINTEDANIB:Renal cancer:0.67), (NIRAPARIB:Renal cancer:0.77), (OLAPARIB:Renal cancer:0.57), (OLODATEROL HYDROCHLORIDETIOTROPIUM BROMIDE MONOHYDRATE:Renal cancer:0.77), (ORPHENADRINE:Renal cancer:0.1), (OSIMERTINIB:Renal cancer:0.07), (OXYBUTYNINOXYBUTYNIN CHLORIDE:Renal cancer:0.05), (PANTOPRAZOLE MAGNESIUM:Renal cancer:1.28), (PARATHYROID HORMONE:Renal cancer:0.19), (PATIROMER:Renal cancer:0.59), (PEGINTERFERON BETA-1A:Renal cancer:0.43), (PEMETREXED:Renal cancer:0.56), (PENTOSAN POLYSULFATE SODIUM:Renal cancer:0.16), (PERTUZUMAB:Renal cancer:0.41), (PHENPROCOUMON:Renal cancer:0.14), (PIMECROLIMUS:Renal cancer:0.29), (PIRFENIDONE:Renal cancer:0.31), (PIROXICAM:Renal cancer:0.54), (POLYETHYLENE GLYCOL 4000:Renal cancer:0.26), (PROGESTERONE:Renal cancer:0.16), (RAMUCIRUMAB:Renal cancer:0.4), (RETINOL:Renal cancer:0.05), (RIVASTIGMINE:Renal cancer:0.31), (RUCAPARIB CAMSYLATE:Renal cancer:0.03), (SALMETEROL XINAFOATE:Renal cancer:0.12), (SECUKINUMAB:Renal cancer:0.43), (SIPULEUCEL-T:Renal cancer:0.23), (SITAGLIPTIN:Renal cancer:0.88), (SODIUM PHOSPHATE, DIBASIC\SODIUM PHOSPHATE, MONOBASIC:Renal cancer:0.4), (TAMOXIFEN CITRATE:Renal cancer:0.16), (TEDUGLUTIDE\WATER:Renal cancer:0.02), (TELOTRISTAT ETHYL:Renal cancer:0.5), (TERBINAFINE HYDROCHLORIDE:Renal cancer:0.68), (TERIFLUNOMIDE:Renal cancer:0.69), (TETRACYCLINE:Renal cancer:0.93), (TINZAPARIN:Renal cancer:0.13), (TIOTROPIUM:Renal cancer:0.6), (TIPIRACIL HYDROCHLORIDETRIFLURIDINE:Renal cancer:0.48), (TOCILIZUMAB:Renal cancer:0.15), (TOCOPHEROL:Renal cancer:0.36), (TRAMETINIB:Renal cancer:0.33), (UMECLIDINIUM BROMIDE:Renal cancer:0.22), (VALGANCICLOVIR HYDROCHLORIDE:Renal cancer:0.47), (VEMURAFENIB:Renal cancer:0.85), (VILAZODONE HYDROCHLORIDE:Renal cancer:0.25), (ZAFIRLUKAST:Renal cancer:0.26), (ZONISAMIDE:Renal cancer:0.23), (ACARBOSE:Renal cancer metastatic:0.15), (AMPHETAMINE ASPARTATEAMPHETAMINE SULFATEDEXTROAMPHETAMINE SACCHARATEDEXTROAMPHETAMINE SULFATE:Renal cancer metastatic:0.04), (BISACODYL:Renal cancer metastatic:0.22), (BUMETANIDE:Renal cancer metastatic:0.3), (CANDESARTAN:Renal cancer metastatic:1.28), (CANDESARTAN CILEXETILHYDROCHLOROTHIAZIDE:Renal cancer metastatic:0.77), (CARVEDILOL PHOSPHATE:Renal cancer metastatic:0.13), (CERTOLIZUMAB PEGOL:Renal cancer metastatic:0.67), (CHOLESTYRAMINE:Renal cancer metastatic:0.29), (CLINDAMYCINCLINDAMYCIN PHOSPHATE:Renal cancer metastatic:0.36), (CLOTRIMAZOLE:Renal cancer metastatic:0.35), (COLESEVELAM HYDROCHLORIDE:Renal cancer metastatic:0.04), (DARBEPOETIN ALFA:Renal cancer metastatic:0.23), (DASATINIB:Renal cancer metastatic:0.62), (DEVICE:Renal cancer metastatic:0.18), (DEXLANSOPRAZOLE:Renal cancer metastatic:1.12), (DOFETILIDE:Renal cancer metastatic:0.11), (DOXYCYCLINE:Renal cancer metastatic:0.01), (EPINEPHRINE:Renal cancer metastatic:0.11), (EPLERENONE:Renal cancer metastatic:0.05), (ESZOPICLONE:Renal cancer metastatic:0.46), (EZETIMIBESIMVASTATIN:Renal cancer metastatic:0.39), (FESOTERODINE FUMARATE:Renal cancer metastatic:0.2), (FLECAINIDE:Renal cancer metastatic:0.48), (FLUOXETINEFLUOXETINE HYDROCHLORIDE:Renal cancer metastatic:0), (FLUTICASONE:Renal cancer metastatic:0.15), (HYDROMORPHONE:Renal cancer metastatic:0.02), (HYDROXYZINE PAMOATE:Renal cancer metastatic:0.19), (ILOPROST:Renal cancer metastatic:0.1), (IMATINIB MESYLATE:Renal cancer metastatic:0.27), (IPRATROPIUM:Renal cancer metastatic:0.11), (LEDIPASVIRSOFOSBUVIR:Renal cancer metastatic:0.42), (LERCANIDIPINE HYDROCHLORIDE:Renal cancer metastatic:0.49), (METAXALONE:Renal cancer metastatic:0.35), (METHOCARBAMOL:Renal cancer metastatic:0.08), (METHYLPREDNISOLONE ACETATE:Renal cancer metastatic:0.27), (METHYLPREDNISOLONE SODIUM SUCCINATE:Renal cancer metastatic:0.4), (MIDODRINE:Renal cancer metastatic:0.1), (NITROFURANTOIN:Renal cancer metastatic:0.46), (PANTOPRAZOLE MAGNESIUM:Renal cancer metastatic:0.42), (PENICILLIN:Renal cancer metastatic:0.15), (RABEPRAZOLE:Renal cancer metastatic:0.09), (RANOLAZINE:Renal cancer metastatic:1.15), (RISEDRONATE SODIUM:Renal cancer metastatic:0.5), (SACUBITRILVALSARTAN:Renal cancer metastatic:0.06), (SECUKINUMAB:Renal cancer metastatic:0.04), (SODIUM CHLORIDE:Renal cancer metastatic:0.04), (SODIUM OXYBATE:Renal cancer metastatic:0.16), (SOTALOL:Renal cancer metastatic:0.8), (TERIFLUNOMIDE:Renal cancer metastatic:0.01), (TETRACYCLINE:Renal cancer metastatic:0), (VARENICLINE TARTRATE:Renal cancer metastatic:0.01), (AMIODARONE HYDROCHLORIDE:Renal cancer stage IV:0.03), (AMITRIPTYLINE:Renal cancer stage IV:0.15), (ANASTROZOLE:Renal cancer stage IV:0.24), (CALCIUM ACETATE:Renal cancer stage IV:0.18), (CALCIUM CARBONATECHOLECALCIFEROL:Renal cancer stage IV:0.23), (CANDESARTAN:Renal cancer stage IV:0.38), (CANDESARTAN CILEXETIL:Renal cancer stage IV:0.27), (CELECOXIB:Renal cancer stage IV:0.47), (CERTOLIZUMAB PEGOL:Renal cancer stage IV:0.2), (CHOLESTYRAMINE:Renal cancer stage IV:0.2), (CLINDAMYCINCLINDAMYCIN PHOSPHATE:Renal cancer stage IV:0.24), (DEVICE:Renal cancer stage IV:0.07), (DEXLANSOPRAZOLE:Renal cancer stage IV:1.15), (ENOXAPARIN:Renal cancer stage IV:0.13), (EPINEPHRINE:Renal cancer stage IV:0.28), (ESZOPICLONE:Renal cancer stage IV:0.04), (EVOLOCUMAB:Renal cancer stage IV:0.36), (FLUTICASONE:Renal cancer stage IV:0.13), (IBRUTINIB:Renal cancer stage IV:0.7), (LANSOPRAZOLE:Renal cancer stage IV:0.62), (LENALIDOMIDE:Renal cancer stage IV:0.57), (LINAGLIPTIN:Renal cancer stage IV:0.33), (METAXALONE:Renal cancer stage IV:0.41), (METHYLPREDNISOLONE SODIUM SUCCINATE:Renal cancer stage IV:0.07), (MOXIFLOXACIN HYDROCHLORIDE:Renal cancer stage IV:0.01), (POLYETHYLENE GLYCOLS:Renal cancer stage IV:0.28), (PRIMIDONE:Renal cancer stage IV:0.01), (QUETIAPINE FUMARATE:Renal cancer stage IV:0.01), (RALOXIFENE HYDROCHLORIDE:Renal cancer stage IV:0.08), (SACUBITRILVALSARTAN:Renal cancer stage IV:0.04), (SOTALOL:Renal cancer stage IV:0.11), (TERIFLUNOMIDE:Renal cancer stage IV:0.34), (TIOTROPIUM BROMIDE MONOHYDRATE:Renal cancer stage IV:0.6), (TOPIRAMATE:Renal cancer stage IV:0.52), (UMECLIDINIUM BROMIDEVILANTEROL TRIFENATATE:Renal cancer stage IV:0.18), (ACETAMINOPHEN\CAFFEINE:Renal cell carcinoma:0.03), (ACLIDINIUM BROMIDE:Renal cell carcinoma:0.08), (ADO-TRASTUZUMAB EMTANSINE:Renal cell carcinoma:1.04), (AFATINIB:Renal cell carcinoma:0.33), (AGALSIDASE BETA:Renal cell carcinoma:0.26), (ALCOHOL:Renal cell carcinoma:0.28), (ALISKIREN:Renal cell carcinoma:0.02), (AMANTADINE:Renal cell carcinoma:0.15), (AMANTADINE HYDROCHLORIDE:Renal cell carcinoma:0.02), (AMILORIDE HYDROCHLORIDEHYDROCHLOROTHIAZIDE:Renal cell carcinoma:0.32), (AMISULPRIDE:Renal cell carcinoma:0.19), (AMLODIPINE BESYLATEHYDROCHLOROTHIAZIDEOLMESARTAN MEDOXOMIL:Renal cell carcinoma:0.02), (AMLODIPINEVALSARTAN:Renal cell carcinoma:0.46), (ARMODAFINIL:Renal cell carcinoma:0.76), (BETAMETHASONE DIPROPIONATECLOTRIMAZOLE:Renal cell carcinoma:0.12), (BILASTINE:Renal cell carcinoma:0.08), (BIMATOPROST:Renal cell carcinoma:0.6), (BUDESONIDEFORMOTEROL FUMARATE:Renal cell carcinoma:0.39), (CALCITONIN:Renal cell carcinoma:0.47), (CALCIUM\MAGNESIUM:Renal cell carcinoma:0.06), (CANAGLIFLOZINMETFORMIN HYDROCHLORIDE:Renal cell carcinoma:0.41), (CARBIMAZOLE:Renal cell carcinoma:0.47), (CARFILZOMIB:Renal cell carcinoma:1.29), (CEFAZOLIN:Renal cell carcinoma:0.03), (CEFDINIR:Renal cell carcinoma:0.04), (CEFPODOXIME PROXETIL:Renal cell carcinoma:0.01), (CEFUROXIME AXETIL:Renal cell carcinoma:0.04), (CHLORDIAZEPOXIDE HYDROCHLORIDECLIDINIUM BROMIDE:Renal cell carcinoma:0.01), (CICLESONIDE:Renal cell carcinoma:0.08), (CILASTATIN SODIUMIMIPENEM:Renal cell carcinoma:0.75), (CIPROFIBRATE:Renal cell carcinoma:0.04), (CLOBAZAM:Renal cell carcinoma:0.21), (CORTICOTROPIN:Renal cell carcinoma:0.21), (DABIGATRAN:Renal cell carcinoma:0.66), (DABRAFENIB:Renal cell carcinoma:0.63), (DACLIZUMAB:Renal cell carcinoma:0.23), (DANAZOL:Renal cell carcinoma:0.82), (DAPAGLIFLOZIN PROPANEDIOL:Renal cell carcinoma:0.1), (DASATINIB:Renal cell carcinoma:1.7), (DEGARELIX:Renal cell carcinoma:0.1), (DESONIDE:Renal cell carcinoma:0.33), (DEVICE:Renal cell carcinoma:0.11), (DEXAMETHASONE ACETATE:Renal cell carcinoma:0.87), (DOFETILIDE:Renal cell carcinoma:0.77), (DONEPEZIL:Renal cell carcinoma:0.17), (DOXEPIN:Renal cell carcinoma:0.59), (DOXEPIN HYDROCHLORIDE:Renal cell carcinoma:0.22), (DRONEDARONE:Renal cell carcinoma:0.43), (DUTASTERIDETAMSULOSIN HYDROCHLORIDE:Renal cell carcinoma:0.05), (ELETRIPTAN HYDROBROMIDE:Renal cell carcinoma:0.83), (ELOTUZUMAB:Renal cell carcinoma:0.41), (ERIBULIN:Renal cell carcinoma:0.25), (ESTROGENS, CONJUGATEDMEDROXYPROGESTERONE ACETATE:Renal cell carcinoma:0.44), (EVOLOCUMAB:Renal cell carcinoma:0.59), (FENOFIBRIC ACID:Renal cell carcinoma:0.37), (FERROUS SULFATEGLYCINE:Renal cell carcinoma:0.04), (FESOTERODINE FUMARATE:Renal cell carcinoma:1.23), (FLUINDIONE:Renal cell carcinoma:0.45), (FLUOXETINEFLUOXETINE HYDROCHLORIDE:Renal cell carcinoma:0.97), (FLUVOXAMINE MALEATE:Renal cell carcinoma:0.16), (FORMOTEROL:Renal cell carcinoma:0.15), (FORMOTEROL FUMARATE:Renal cell carcinoma:0.67), (FUSIDIC ACID:Renal cell carcinoma:0.67), (GEFITINIB:Renal cell carcinoma:0.37), (GENTAMICIN:Renal cell carcinoma:0.33), (GIMERACILOTERACILTEGAFUR:Renal cell carcinoma:0.66), (GLYCOPYRRONIUMINDACATEROL:Renal cell carcinoma:0.79), (HYDROCODONE BITARTRATEIBUPROFEN:Renal cell carcinoma:0.03), (HYDROCORTISONE ACETATE:Renal cell carcinoma:0.07), (HYDROXYUREA:Renal cell carcinoma:0.51), (ICOSAPENT ETHYL:Renal cell carcinoma:0.01), (IDELALISIB:Renal cell carcinoma:0.44), (ILOPROST:Renal cell carcinoma:0.61), (INDACATEROL:Renal cell carcinoma:0.1), (IRINOTECAN HYDROCHLORIDE:Renal cell carcinoma:0.47), (IXAZOMIB:Renal cell carcinoma:0.38), (LACTOBACILLUS RHAMNOSUS:Renal cell carcinoma:0.3), (LEDIPASVIRSOFOSBUVIR:Renal cell carcinoma:1.02), (LEVALBUTEROL HYDROCHLORIDE:Renal cell carcinoma:1.26), (LINACLOTIDE:Renal cell carcinoma:0.06), (LISDEXAMFETAMINE DIMESYLATE:Renal cell carcinoma:0.41), (LITHIUM:Renal cell carcinoma:0.05), (LITHIUM CARBONATE:Renal cell carcinoma:1.18), (LOMITAPIDE MESYLATE:Renal cell carcinoma:0.15), (METHENAMINE HIPPURATE:Renal cell carcinoma:0.57), (METHYLPREDNISOLONE ACETATE:Renal cell carcinoma:0.74), (MINOCYCLINE HYDROCHLORIDE:Renal cell carcinoma:0.9), (MOXIFLOXACIN:Renal cell carcinoma:0.56), (MYCOPHENOLATE MOFETILMYCOPHENOLATE MOFETIL HYDROCHLORIDE:Renal cell carcinoma:0.51), (NABUMETONE:Renal cell carcinoma:0.53), (NILOTINIB:Renal cell carcinoma:1.7), (NINTEDANIB:Renal cell carcinoma:0.85), (NIRAPARIB:Renal cell carcinoma:0.53), (OLAPARIB:Renal cell carcinoma:0.31), (OLODATEROL HYDROCHLORIDETIOTROPIUM BROMIDE MONOHYDRATE:Renal cell carcinoma:0.96), (OSIMERTINIB:Renal cell carcinoma:0.45), (PACLITAXEL:Renal cell carcinoma:1.46), (PALONOSETRON HYDROCHLORIDE:Renal cell carcinoma:0.35), (PATIROMER:Renal cell carcinoma:0.33), (PEGINTERFERON BETA-1A:Renal cell carcinoma:0.16), (PERTUZUMAB:Renal cell carcinoma:0.19), (PILOCARPINE:Renal cell carcinoma:0.08), (PILSICAINIDE HYDROCHLORIDE:Renal cell carcinoma:0.07), (PIRFENIDONE:Renal cell carcinoma:0.13), (POLYETHYLENE GLYCOL 4000:Renal cell carcinoma:0.67), (PONATINIB:Renal cell carcinoma:0.79), (PRAZOSIN:Renal cell carcinoma:0.05), (PROGESTERONE:Renal cell carcinoma:0.35), (PROPAFENONE HYDROCHLORIDE:Renal cell carcinoma:0.28), (PYRIDOSTIGMINE BROMIDE:Renal cell carcinoma:0.05), (RABEPRAZOLE:Renal cell carcinoma:1.05), (RAMUCIRUMAB:Renal cell carcinoma:0.65), (REGORAFENIB:Renal cell carcinoma:0.99), (RETINOL:Renal cell carcinoma:0.26), (RIFAXIMIN:Renal cell carcinoma:0.15), (RIZATRIPTAN BENZOATE:Renal cell carcinoma:0.67), (SALMETEROL XINAFOATE:Renal cell carcinoma:0.08), (SECUKINUMAB:Renal cell carcinoma:0.22), (SILVER SULFADIAZINE:Renal cell carcinoma:0.24), (SIPULEUCEL-T:Renal cell carcinoma:0.01), (SODIUM OXYBATE:Renal cell carcinoma:0.69), (SOTALOL HYDROCHLORIDE:Renal cell carcinoma:0.08), (SUMATRIPTAN SUCCINATE:Renal cell carcinoma:0.04), (SUMATRIPTANSUMATRIPTAN SUCCINATE:Renal cell carcinoma:0.46), (TERBUTALINE:Renal cell carcinoma:0.04), (TERIFLUNOMIDE:Renal cell carcinoma:0.19), (TETRACYCLINE:Renal cell carcinoma:0.9), (TIMOLOL MALEATE:Renal cell carcinoma:0.82), (TRAMETINIB:Renal cell carcinoma:0.68), (TROSPIUM CHLORIDE:Renal cell carcinoma:0.25), (UMECLIDINIUM BROMIDE:Renal cell carcinoma:0.18), (VALGANCICLOVIR HYDROCHLORIDE:Renal cell carcinoma:0.36), (VANCOMYCIN HYDROCHLORIDE:Renal cell carcinoma:0.42), (VEMURAFENIB:Renal cell carcinoma:0.72), (VILAZODONE HYDROCHLORIDE:Renal cell carcinoma:0.08), (VINORELBINE TARTRATE:Renal cell carcinoma:0.58), (UNSPECIFIED INGREDIENT:Renal cell carcinoma stage IV:0.65), (GADOLINIUM:Renal cyst:0.02), (.ALPHA.1-PROTEINASE INHIBITOR HUMAN:Renal disorder:0.07), (ABIRATERONE ACETATE:Renal disorder:0.12), (ACETAMINOPHENOXYCODONEOXYCODONE HYDROCHLORIDE:Renal disorder:0.81), (ACETAMINOPHENPROPOXYPHENE HYDROCHLORIDE:Renal disorder:0.17), (ACETAMINOPHENTRAMADOL:Renal disorder:0.21), (ACETAZOLAMIDE:Renal disorder:1.11), (ALCOHOL:Renal disorder:0.36), (ALENDRONIC ACID:Renal disorder:0.38), (ALISKIREN HEMIFUMARATE:Renal disorder:0.66), (AMINO ACIDSDEXTROSEELECTROLYTES NOS:Renal disorder:0.02), (AMISULPRIDE:Renal disorder:0.19), (AMLODIPINE BESYLATEOLMESARTAN MEDOXOMIL:Renal disorder:0.44), (BETAMETHASONE DIPROPIONATECALCIPOTRIENECALCIPOTRIENE HYDRATE:Renal disorder:0.23), (BROMAZEPAM:Renal disorder:0.56), (BUTYLSCOPOLAMINE BROMIDE:Renal disorder:0.03), (CALCITONIN SALMON:Renal disorder:0.39), (CALCIUM\MAGNESIUM\ZINC:Renal disorder:0.59), (CANAKINUMAB:Renal disorder:0.44), (CANDESARTAN:Renal disorder:1.9), (CANDESARTAN CILEXETILHYDROCHLOROTHIAZIDE:Renal disorder:1.37), (CARVEDILOL PHOSPHATE:Renal disorder:1.75), (CEFUROXIME:Renal disorder:0.03), (CHLORDIAZEPOXIDE HYDROCHLORIDECLIDINIUM BROMIDE:Renal disorder:0.29), (CLARITHROMYCIN:Renal disorder:0.28), (CODEINE:Renal disorder:0.66), (CODEINE PHOSPHATEGUAIFENESIN:Renal disorder:0.09), (DABIGATRAN:Renal disorder:0.47), (DACLIZUMAB:Renal disorder:0.78), (DANAZOL:Renal disorder:0.58), (DESLORATADINE:Renal disorder:1.02), (DESONIDE:Renal disorder:0.27), (DIGITOXIN:Renal disorder:0.16), (DORZOLAMIDE HYDROCHLORIDE:Renal disorder:0.04), (DOXEPIN HYDROCHLORIDE:Renal disorder:0.53), (DOXYCYCLINE HYCLATE:Renal disorder:0.05), (DRONEDARONE:Renal disorder:0.48), (EMPAGLIFLOZINLINAGLIPTIN:Renal disorder:1.51), (ESOMEPRAZOLE MAGNESIUMNAPROXEN:Renal disorder:0.29), (ESTROGENS, CONJUGATEDMEDROXYPROGESTERONE ACETATE:Renal disorder:0.61), (ESTRONESUS SCROFA OVARY:Renal disorder:0.03), (ETORICOXIB:Renal disorder:0.58), (FERRIC HYDROXIDE:Renal disorder:0.07), (FISH OIL\OMEGA-3 FATTY ACIDS:Renal disorder:0.77), (FLUTICASONE FUROATE:Renal disorder:0.57), (FORMOTEROL:Renal disorder:0.09), (FORMOTEROL FUMARATE\MOMETASONE FUROATE:Renal disorder:0.1), (FUSIDIC ACID:Renal disorder:0.52), (GLYCOPYRRONIUMINDACATEROL:Renal disorder:0.63), (HYDROCHLOROTHIAZIDEIRBESARTAN:Renal disorder:0.95), (HYDROCHLOROTHIAZIDELOSARTAN:Renal disorder:0.03), (HYDROCODONE BITARTRATEIBUPROFEN:Renal disorder:0.16), (HYOSCYAMINE:Renal disorder:1.15), (ILOPROST:Renal disorder:1.02), (INDAPAMIDEPERINDOPRIL:Renal disorder:0.35), (IVABRADINE HYDROCHLORIDE:Renal disorder:0.03), (IXAZOMIB:Renal disorder:0.05), (KETOROLACKETOROLAC TROMETHAMINE:Renal disorder:0.26), (LACTASE:Renal disorder:0.35), (LANREOTIDE ACETATE:Renal disorder:0.79), (LIDOCAINE HYDROCHLORIDE:Renal disorder:1), (LIOTHYRONINE:Renal disorder:0.03), (LISDEXAMFETAMINE DIMESYLATE:Renal disorder:0.94), (LITHIUM:Renal disorder:0.14), (LOMITAPIDE MESYLATE:Renal disorder:0.92), (MEROPENEM:Renal disorder:0.24), (METFORMIN HYDROCHLORIDEROSIGLITAZONE MALEATE:Renal disorder:0.11), (MILRINONE:Renal disorder:0.12), (MUPIROCIN:Renal disorder:0.44), (NALOXONE HYDROCHLORIDEOXYCODONE HYDROCHLORIDE:Renal disorder:1.1), (NINTEDANIB:Renal disorder:0.65), (OLMESARTAN:Renal disorder:0.15), (OLODATEROL HYDROCHLORIDETIOTROPIUM BROMIDE MONOHYDRATE:Renal disorder:0.5), (OMEPRAZOLE SODIUM:Renal disorder:0.12), (OXAZEPAM:Renal disorder:0.15), (OXYBUTYNINOXYBUTYNIN CHLORIDE:Renal disorder:0.04), (PANTOPRAZOLE MAGNESIUM:Renal disorder:1.54), (PEGINTERFERON ALFA-2B:Renal disorder:0.02), (PEGINTERFERON BETA-1A:Renal disorder:0.95), (PEMETREXED DISODIUM:Renal disorder:0.04), (PENTOSAN POLYSULFATE SODIUM:Renal disorder:0.14), (PHENYTOIN SODIUM:Renal disorder:0.26), (PRAZOSIN HYDROCHLORIDE:Renal disorder:0.31), (PREDNISOLONEPREDNISOLONE ACETATEPREDNISOLONE SODIUM SUCCINATE:Renal disorder:0.54), (RETINOL:Renal disorder:0.01), (RIOCIGUAT:Renal disorder:0.73), (RIZATRIPTAN:Renal disorder:0.17), (RIZATRIPTAN BENZOATE:Renal disorder:0.99), (ROSIGLITAZONE MALEATE:Renal disorder:0.22), (CEREVISIAE:Renal disorder:0.33), (SALMETEROL XINAFOATE:Renal disorder:0.22), (SITAGLIPTIN:Renal disorder:0.55), (TERBINAFINE HYDROCHLORIDE:Renal disorder:0.67), (TOBRAMYCIN:Renal disorder:0.12), (UMECLIDINIUM BROMIDE:Renal disorder:0.19), (UMECLIDINIUM BROMIDEVILANTEROL TRIFENATATE:Renal disorder:1.27), (VALGANCICLOVIR HYDROCHLORIDE:Renal disorder:1.09), (VANCOMYCIN HYDROCHLORIDE:Renal disorder:0.06), (VILAZODONE HYDROCHLORIDE:Renal disorder:0.58), (ACETAMINOPHENOXYCODONEOXYCODONE HYDROCHLORIDE:Renal failure:0.24), (ADO-TRASTUZUMAB EMTANSINE:Renal failure:0.2), (ALCOHOL:Renal failure:0.13), (ALUMINUM HYDROXIDEDIMETHICONEMAGNESIUM HYDROXIDE:Renal failure:0.11), (AMANTADINE:Renal failure:0.35), (AMISULPRIDE:Renal failure:0.47), (AMOXICILLINCLAVULANIC ACID:Renal failure:0.37), (AMPHETAMINE ASPARTATEAMPHETAMINE SULFATEDEXTROAMPHETAMINE SACCHARATEDEXTROAMPHETAMINE SULFATE:Renal failure:0.44), (ANASTROZOLE:Renal failure:1.17), (AZACITIDINE:Renal failure:0.16), (BENAZEPRILBENAZEPRIL HYDROCHLORIDE:Renal failure:0.41), (BENZONATATE:Renal failure:0.01), (BUDESONIDEFORMOTEROL FUMARATE:Renal failure:0.4), (BUPRENORPHINE HYDROCHLORIDE:Renal failure:0.8), (BUPROPION:Renal failure:0.57), (BUSPIRONE HYDROCHLORIDE:Renal failure:0.47), (CALCIUM CARBONATEVITAMIN D:Renal failure:0.25), (CALCIUMCHOLECALCIFEROL:Renal failure:0.13), (CANAGLIFLOZINMETFORMIN HYDROCHLORIDE:Renal failure:0.27), (CANDESARTAN CILEXETILHYDROCHLOROTHIAZIDE:Renal failure:1.54), (CARVEDILOL PHOSPHATE:Renal failure:0.87), (CASPOFUNGIN:Renal failure:0.29), (CEFEPIMECEFEPIME HYDROCHLORIDE:Renal failure:0.16), (CIMETIDINE:Renal failure:0.02), (CLOBAZAM:Renal failure:0.26), (CLOBETASOL:Renal failure:0.12), (CYCLOBENZAPRINE:Renal failure:0.54), (DABIGATRAN:Renal failure:0.71), (DACLIZUMAB:Renal failure:0.3), (DALTEPARIN:Renal failure:0.41), (DANAZOL:Renal failure:0.48), (DASATINIB:Renal failure:1.63), (DEFLAZACORT:Renal failure:0.03), (DIPYRIDAMOLE:Renal failure:0.41), (DIPYRONE:Renal failure:0.01), (DOFETILIDE:Renal failure:0.63), (DRONEDARONE:Renal failure:0.06), (DULOXETINE:Renal failure:0.56), (EDOXABAN TOSYLATE:Renal failure:1.17), (ELETRIPTAN HYDROBROMIDE:Renal failure:0.29), (ELTROMBOPAG OLAMINE:Renal failure:0.18), (EMPAGLIFLOZINLINAGLIPTIN:Renal failure:0.1), (EPINASTINE HYDROCHLORIDE:Renal failure:0.01), (EPIRUBICIN:Renal failure:0.41), (ETORICOXIB:Renal failure:0.77), (EVOLOCUMAB:Renal failure:1.11), (FENOTEROL HYDROBROMIDEIPRATROPIUM BROMIDE:Renal failure:0.09), (FERRIC HYDROXIDE:Renal failure:0.15), (FLUDROCORTISONE ACETATE:Renal failure:0.05), (FLUTICASONE FUROATEVILANTEROL TRIFENATATE:Renal failure:0.17), (FONDAPARINUX:Renal failure:0.03), (FUSIDIC ACID:Renal failure:0.27), (GADOLINIUM:Renal failure:0.33), (GLYCOPYRRONIUMINDACATEROL:Renal failure:0.81), (GUAIFENESIN:Renal failure:0.31), (HYDROXYCHLOROQUINE DIPHOSPHATE:Renal failure:0.17), (HYDROXYZINE PAMOATE:Renal failure:0.77), (IDELALISIB:Renal failure:0.14), (INDAPAMIDEPERINDOPRIL:Renal failure:1.42), (IPRATROPIUM:Renal failure:1.13), (IRINOTECAN HYDROCHLORIDE:Renal failure:0.01), (LABETALOL HYDROCHLORIDE:Renal failure:0.67), (LANREOTIDE ACETATE:Renal failure:0.08), (LENOGRASTIM:Renal failure:0.14), (LEVALBUTEROL HYDROCHLORIDE:Renal failure:0.74), (LIDOCAINE HYDROCHLORIDE:Renal failure:0.46), (LIDOCAINEPRILOCAINE:Renal failure:0.46), (MAGNESIUM ASPARTATE HYDROCHLORIDE:Renal failure:0.12), (MELATONIN:Renal failure:0.43), (METAXALONE:Renal failure:0.45), (METFORMIN HYDROCHLORIDEVILDAGLIPTIN:Renal failure:0.16), (METHENAMINE HIPPURATE:Renal failure:0.25), (METHENOLONE ENANTHATE:Renal failure:0.13), (METHOCARBAMOL:Renal failure:0.39), (NAPROXEN SODIUM:Renal failure:0.55), (NATEGLINIDE:Renal failure:0.21), (NINTEDANIB:Renal failure:0.16), (NOREPINEPHRINE:Renal failure:0.39), (NOREPINEPHRINE BITARTRATE:Renal failure:0.34), (NORTRIPTYLINE:Renal failure:0.42), (OLODATEROL HYDROCHLORIDETIOTROPIUM BROMIDE MONOHYDRATE:Renal failure:0.07), (OSELTAMIVIR:Renal failure:0.63), (PAROXETINEPAROXETINE HYDROCHLORIDE:Renal failure:0.8), (PEGINTERFERON BETA-1A:Renal failure:0.34), (PEMETREXED DISODIUM:Renal failure:0.09), (PITAVASTATIN CALCIUM:Renal failure:0.53), (PLANTAGO SEED:Renal failure:0.19), (PONATINIB:Renal failure:0.82), (POSACONAZOLE:Renal failure:0.79), (PRIMIDONE:Renal failure:0.69), (PROPAFENONE HYDROCHLORIDE:Renal failure:0.13), (PYRIDOSTIGMINE BROMIDE:Renal failure:0.04), (RALOXIFENE HYDROCHLORIDE:Renal failure:0.18), (RIZATRIPTAN BENZOATE:Renal failure:0.19), (ROFLUMILAST:Renal failure:0.74), (SCOPOLAMINE:Renal failure:0.43), (SELEXIPAG:Renal failure:0.53), (SODIUM PHOSPHATE, DIBASIC\SODIUM PHOSPHATE, MONOBASIC:Renal failure:0.51), (SOTALOL:Renal failure:1.49), (SUMATRIPTAN:Renal failure:0.26), (TERBUTALINE:Renal failure:0.11), (TERIFLUNOMIDE:Renal failure:0.42), (TETRACYCLINE:Renal failure:0.34), (TINZAPARIN:Renal failure:0.1), (TIZANIDINE:Renal failure:0.46), (TOPIRAMATE:Renal failure:1.55), (TROSPIUM CHLORIDE:Renal failure:0.26), (VARENICLINE TARTRATE:Renal failure:0.6), (VASOPRESSIN:Renal failure:0.11), (VINCRISTINE SULFATE:Renal failure:0.12), (WARFARIN POTASSIUM:Renal failure:0.37), (CANDESARTAN:Renal failure chronic:0.19), (DILTIAZEM:Renal failure chronic:0.09), (EVOLOCUMAB:Renal failure chronic:0.17), (IBRUTINIB:Renal failure chronic:0.22), (IBUPROFEN:Renal failure chronic:0.16), (LEDIPASVIRSOFOSBUVIR:Renal failure chronic:0.04), (NAPROXEN:Renal failure chronic:0.03), (PANTOPRAZOLE:Renal failure chronic:0.18), (PRAVASTATIN:Renal failure chronic:0.32), (RANOLAZINE:Renal failure chronic:0.34), (SACUBITRILVALSARTAN:Renal failure chronic:0.24), (AMIODARONE HYDROCHLORIDE:Renal impairment:0.66), (ANASTROZOLE:Renal impairment:0.4), (BENAZEPRIL HYDROCHLORIDE:Renal impairment:0.37), (BENDAMUSTINE:Renal impairment:0.23), (CANAGLIFLOZIN:Renal impairment:0.02), (CASPOFUNGIN:Renal impairment:0.06), (CERTOLIZUMAB PEGOL:Renal impairment:0.46), (CHOLESTYRAMINE:Renal impairment:0.23), (CIPROFLOXACIN HYDROCHLORIDE:Renal impairment:0.21), (CLINDAMYCINCLINDAMYCIN PHOSPHATE:Renal impairment:0.64), (CLOTRIMAZOLE:Renal impairment:0.22), (CLOZAPINE:Renal impairment:0.34), (CYCLOBENZAPRINE:Renal impairment:0.02), (DALFAMPRIDINE:Renal impairment:0.31), (DEVICE:Renal impairment:0.38), (DILTIAZEM HYDROCHLORIDE:Renal impairment:1.78), (DIVALPROEX SODIUM:Renal impairment:0.34), (DOCUSATE SODIUM:Renal impairment:0.77), (DUTASTERIDE:Renal impairment:0.22), (EVOLOCUMAB:Renal impairment:0.51), (FLECAINIDE:Renal impairment:0.23), (HYDROMORPHONE:Renal impairment:0.06), (ILOPROST:Renal impairment:0.29), (IMATINIB MESYLATE:Renal impairment:0.32), (IPRATROPIUM:Renal impairment:0.15), (LATANOPROST:Renal impairment:0.03), (LEDIPASVIRSOFOSBUVIR:Renal impairment:1.08), (LIDOCAINEPRILOCAINE:Renal impairment:0.04), (NYSTATIN:Renal impairment:0.42), (OMEGA-3-ACID ETHYL ESTERS:Renal impairment:0.23), (OXCARBAZEPINE:Renal impairment:0.28), (PAZOPANIB HYDROCHLORIDE:Renal impairment:0.36), (PHENOBARBITAL:Renal impairment:0.01), (POLYETHYLENE GLYCOLS:Renal impairment:0.65), (RANOLAZINE:Renal impairment:0.98), (SACUBITRILVALSARTAN:Renal impairment:0.81), (SECUKINUMAB:Renal impairment:0.44), (SEVELAMER HYDROCHLORIDE:Renal impairment:0.02), (SOTALOL:Renal impairment:0.22), (TOPIRAMATE:Renal impairment:0.66), (UNSPECIFIED INGREDIENT:Renal impairment:1.16), (VANCOMYCIN HYDROCHLORIDE:Renal impairment:0.13), (VINCRISTINE SULFATE:Renal impairment:0.33), (.ALPHA.1-PROTEINASE INHIBITOR HUMAN:Renal transplant:0.52), (ACETAMINOPHENPROPOXYPHENE HYDROCHLORIDE:Renal transplant:0.24), (ADO-TRASTUZUMAB EMTANSINE:Renal transplant:0.25), (ALISKIREN:Renal transplant:0.6), (AMANTADINE:Renal transplant:0.08), (AMINO ACIDSDEXTROSEELECTROLYTES NOS:Renal transplant:0.06), (AMINO ACIDSDEXTROSEELECTROLYTES NOSSOYBEAN OILVITAMINS:Renal transplant:0.05), (AMISULPRIDE:Renal transplant:0.78), (AMLODIPINE BESYLATEHYDROCHLOROTHIAZIDEOLMESARTAN MEDOXOMIL:Renal transplant:0.01), (AMLODIPINE BESYLATEHYDROCHLOROTHIAZIDEVALSARTAN:Renal transplant:0.34), (AMLODIPINE BESYLATEOLMESARTAN MEDOXOMIL:Renal transplant:0.05), (AMLODIPINEVALSARTAN:Renal transplant:0.55), (AMMONIUM LACTATE:Renal transplant:0.33), (APREPITANT:Renal transplant:0.35), (ASPIRINCLOPIDOGREL:Renal transplant:0.15), (AVOBENZONEHOMOSALATEOCTISALATE:Renal transplant:0.68), (AZELASTINE HYDROCHLORIDE:Renal transplant:0.02), (BECLOMETHASONE:Renal transplant:0.31), (BENAZEPRILBENAZEPRIL HYDROCHLORIDE:Renal transplant:0.97), (BENDAMUSTINE HYDROCHLORIDE:Renal transplant:0.17), (BISACODYLGLYCERINMINERAL OILSODIUM PHOSPHATE, DIBASIC\SODIUM PHOSPHATE, MONOBASIC:Renal transplant:0.71), (BUDESONIDEFORMOTEROL FUMARATE:Renal transplant:0.45), (BUPRENORPHINE HYDROCHLORIDE:Renal transplant:0.41), (CANAGLIFLOZINMETFORMIN HYDROCHLORIDE:Renal transplant:0.95), (CARFILZOMIB:Renal transplant:1.26), (CARISOPRODOL:Renal transplant:0.14), (CARVEDILOL PHOSPHATE:Renal transplant:1.18), (CEFOTAXIMECEFOTAXIME SODIUM:Renal transplant:0.46), (CELECOXIB:Renal transplant:1.44), (CERTOLIZUMAB PEGOL:Renal transplant:1.32), (CHLORDIAZEPOXIDE HYDROCHLORIDECLIDINIUM BROMIDE:Renal transplant:0.02), (CHLORPROMAZINE:Renal transplant:0.16), (CHONDROITIN SULFATE (BOVINE)GLUCOSAMINE HYDROCHLORIDE:Renal transplant:0.02), (COLISTIMETHATE SODIUM:Renal transplant:0.37), (CRIZOTINIB:Renal transplant:0.21), (CYAMEMAZINE:Renal transplant:0.37), (CYPROHEPTADINE:Renal transplant:0.49), (DANAZOL:Renal transplant:0.81), (DAPAGLIFLOZIN PROPANEDIOL:Renal transplant:0.61), (DARATUMUMAB:Renal transplant:0.22), (DESLORATADINE:Renal transplant:0.99), (DEXAMETHASONE ACETATE:Renal transplant:0.72), (DEXMEDETOMIDINE HYDROCHLORIDE:Renal transplant:1.28), (DEXTROAMPHETAMINE SULFATE:Renal transplant:0.3), (DIGITOXIN:Renal transplant:0.31), (DOFETILIDE:Renal transplant:0.78), (DOXEPIN HYDROCHLORIDE:Renal transplant:0.11), (DUTASTERIDETAMSULOSIN HYDROCHLORIDE:Renal transplant:0.04), (EDOXABAN TOSYLATE:Renal transplant:0.95), (ELOTUZUMAB:Renal transplant:1.49), (EMPAGLIFLOZINLINAGLIPTIN:Renal transplant:0.37), (EPINASTINE HYDROCHLORIDE:Renal transplant:0.05), (ETHACRYNIC ACID:Renal transplant:0.06), (ETORICOXIB:Renal transplant:0.39), (EVOLOCUMAB:Renal transplant:1.43), (FERRIC HYDROXIDE:Renal transplant:0.17), (FERROUS SULFATE ANHYDROUS:Renal transplant:0.02), (FESOTERODINE FUMARATE:Renal transplant:0.88), (FLUTICASONE FUROATE:Renal transplant:0.35), (FLUTICASONE FUROATESALMETEROL XINAFOATE:Renal transplant:1.04), (FLUVOXAMINE MALEATE:Renal transplant:0.15), (FONDAPARINUX:Renal transplant:0.41), (FONDAPARINUX SODIUM:Renal transplant:0.21), (FORMOTEROL:Renal transplant:0.51), (FORMOTEROL FUMARATE\MOMETASONE FUROATE:Renal transplant:0.42), (FOSPHENYTOINFOSPHENYTOIN SODIUM:Renal transplant:0.31), (FUSIDIC ACID:Renal transplant:0.39), (GLYCOPYRRONIUMINDACATEROL:Renal transplant:0.87), (HERBALSSAW PALMETTO:Renal transplant:0.4), (HYDROCHLOROTHIAZIDELOSARTAN POTASSIUM:Renal transplant:0.79), (HYDROCHLOROTHIAZIDETELMISARTAN:Renal transplant:0.58), (HYDROXYCHLOROQUINE DIPHOSPHATE:Renal transplant:0.19), (HYDROXYZINE PAMOATE:Renal transplant:1.2), (IDELALISIB:Renal transplant:0.31), (ILOPROST:Renal transplant:0.41), (IMIPRAMINE HYDROCHLORIDE:Renal transplant:0.41), (INTERFERON GAMMA-1B:Renal transplant:0.15), (IOPAMIDOL:Renal transplant:0.25), (IRINOTECAN HYDROCHLORIDE:Renal transplant:0.36), (IXAZOMIB:Renal transplant:0), (KETOPROFEN:Renal transplant:0.23), (LACTASE:Renal transplant:0.11), (LEVOLEUCOVORIN CALCIUM:Renal transplant:0.79), (LEVOSIMENDAN:Renal transplant:0.1), (LIDOCAINEPRILOCAINE:Renal transplant:0.31), (LISDEXAMFETAMINE DIMESYLATE:Renal transplant:0.76), (LOMITAPIDE MESYLATE:Renal transplant:0.24), (MESNA:Renal transplant:0.8), (METAXALONE:Renal transplant:0.97), (METHIMAZOLE:Renal transplant:0.7), (METILDIGOXIN:Renal transplant:0.61), (MUPIROCINMUPIROCIN CALCIUM:Renal transplant:0.64), (NABUMETONE:Renal transplant:0.21), (NALBUPHINE HYDROCHLORIDE:Renal transplant:0.05), (NALOXONE HYDROCHLORIDETILIDINE HYDROCHLORIDE:Renal transplant:0.15), (NAPROXEN:Renal transplant:2.02), (NAPROXEN SODIUM:Renal transplant:0.43), (OLODATEROL HYDROCHLORIDETIOTROPIUM BROMIDE MONOHYDRATE:Renal transplant:0.28), (OLOPATADINE:Renal transplant:0.17), (PAMIDRONATE DISODIUM:Renal transplant:0.17), (PEMETREXED DISODIUM:Renal transplant:0.27), (PHENYTOIN SODIUM:Renal transplant:1.1), (PIMOBENDAN:Renal transplant:0.33), (PIROXICAM:Renal transplant:0.19), (PLASMA PROTEIN FRACTION (HUMAN):Renal transplant:0.2), (PONATINIB:Renal transplant:1.13), (PYRIDOSTIGMINE BROMIDE:Renal transplant:0.44), (QUINAPRILQUINAPRIL HYDROCHLORIDE:Renal transplant:0.08), (REGORAFENIB:Renal transplant:0.18), (RIVASTIGMINE:Renal transplant:0.04), (RIVASTIGMINE TARTRATE:Renal transplant:0.09), (RIZATRIPTAN BENZOATE:Renal transplant:0.34), (ROFLUMILAST:Renal transplant:0.95), (ROPIVACAINE:Renal transplant:0.39), (SECUKINUMAB:Renal transplant:1.08), (SIPULEUCEL-T:Renal transplant:0.28), (SITAGLIPTIN:Renal transplant:0.82), (SODIUM CITRATE:Renal transplant:0.76), (SODIUM FLUORIDE:Renal transplant:0.51), (SUGAMMADEX:Renal transplant:0.38), (SUMATRIPTANSUMATRIPTAN SUCCINATE:Renal transplant:0.84), (THIAMYLAL SODIUM:Renal transplant:0.04), (TINZAPARIN:Renal transplant:0.43), (TINZAPARIN SODIUM:Renal transplant:0.7), (TIZANIDINE:Renal transplant:0.6), (UMECLIDINIUM BROMIDEVILANTEROL TRIFENATATE:Renal transplant:0.96), (VARENICLINE TARTRATE:Renal transplant:1.26), (VITAMIN A:Renal transplant:0.65), (WARFARIN POTASSIUM:Renal transplant:0.29), (ACETAMINOPHENOXYCODONEOXYCODONE HYDROCHLORIDE:Respiratory disorder:0.41), (ACETAMINOPHENPROPOXYPHENE HYDROCHLORIDE:Respiratory disorder:0.18), (ALCOHOL:Respiratory disorder:0.38), (ALENDRONIC ACID:Respiratory disorder:0.17), (ALUMINUM HYDROXIDEDIMETHICONEMAGNESIUM HYDROXIDE:Respiratory disorder:0.04), (AMANTADINE:Respiratory disorder:0.17), (AMISULPRIDE:Respiratory disorder:0.53), (AMLODIPINE BESYLATEHYDROCHLOROTHIAZIDEOLMESARTAN MEDOXOMIL:Respiratory disorder:0.12), (BENAZEPRILBENAZEPRIL HYDROCHLORIDE:Respiratory disorder:0.43), (BIOTIN:Respiratory disorder:0.44), (BISACODYL OR DOCUSATE SODIUM:Respiratory disorder:1.05), (CANAGLIFLOZINMETFORMIN HYDROCHLORIDE:Respiratory disorder:0.29), (CAPTOPRIL:Respiratory disorder:0.22), (CEFOTAXIME SODIUM:Respiratory disorder:0.57), (CHLORDIAZEPOXIDE HYDROCHLORIDECLIDINIUM BROMIDE:Respiratory disorder:0.2), (CHLORPHENIRAMINEHYDROCODONE:Respiratory disorder:0.03), (CHONDROITIN SULFATE (BOVINE)GLUCOSAMINE HYDROCHLORIDE:Respiratory disorder:0.32), (CIMETIDINE:Respiratory disorder:0.35), (COLISTIN:Respiratory disorder:0.08), (DABIGATRAN:Respiratory disorder:0.21), (DACLIZUMAB:Respiratory disorder:0.84), (DALFAMPRIDINE:Respiratory disorder:1.31), (DALTEPARIN:Respiratory disorder:0.05), (DALTEPARIN SODIUM:Respiratory disorder:0.1), (DANAZOL:Respiratory disorder:0.34), (DARBEPOETIN ALFA:Respiratory disorder:0.27), (DASATINIB:Respiratory disorder:1.78), (DOCUSATE CALCIUM:Respiratory disorder:0.41), (DOXEPIN HYDROCHLORIDE:Respiratory disorder:0.14), (ECONAZOLE:Respiratory disorder:0.2), (EDOXABAN TOSYLATE:Respiratory disorder:0.33), (EMPAGLIFLOZINLINAGLIPTIN:Respiratory disorder:0.49), (ENOXAPARIN:Respiratory disorder:1.41), (ETORICOXIB:Respiratory disorder:0.08), (EVOLOCUMAB:Respiratory disorder:1.64), (FISH OIL\OMEGA-3 FATTY ACIDS:Respiratory disorder:0.32), (FOSFOMYCIN:Respiratory disorder:0.02), (GENTAMICIN:Respiratory disorder:0.07), (HERBALSTURMERIC:Respiratory disorder:0.19), (HYDROXYCHLOROQUINE DIPHOSPHATE:Respiratory disorder:0.01), (HYOSCYAMINE:Respiratory disorder:0.29), (ILOPROST:Respiratory disorder:0.97), (IMATINIB MESYLATE:Respiratory disorder:0.92), (IMIPRAMINE HYDROCHLORIDE:Respiratory disorder:0.38), (INDOMETHACIN:Respiratory disorder:0.6), (INSULIN GLULISINE:Respiratory disorder:0.1), (KETOROLACKETOROLAC TROMETHAMINE:Respiratory disorder:0.12), (RHAMNOSUS:Respiratory disorder:0.06), (LEDIPASVIRSOFOSBUVIR:Respiratory disorder:0.92), (LERCANIDIPINE:Respiratory disorder:0.85), (LIDOCAINE HYDROCHLORIDE:Respiratory disorder:0.36), (LIDOCAINEPRILOCAINE:Respiratory disorder:0.38), (LOMITAPIDE MESYLATE:Respiratory disorder:0.28), (LUMACAFTOR:Respiratory disorder:0.68), (MEGESTROL ACETATE:Respiratory disorder:0.47), (METHYLPREDNISOLONE ACETATE:Respiratory disorder:0.37), (MIDODRINE:Respiratory disorder:0.37), (NATEGLINIDE:Respiratory disorder:0.29), (OLOPATADINE:Respiratory disorder:0.51), (OMEPRAZOLE SODIUM:Respiratory disorder:0.02), (PEGFILGRASTIM:Respiratory disorder:0), (PEGINTERFERON BETA-1A:Respiratory disorder:0.66), (PENICILLIN:Respiratory disorder:0.8), (PERINDOPRIL:Respiratory disorder:0.62), (PIMECROLIMUS:Respiratory disorder:0.23), (PIROXICAM:Respiratory disorder:0.19), (PRAZOSIN:Respiratory disorder:0.23), (PREDNISOLONEPREDNISOLONE ACETATEPREDNISOLONE SODIUM SUCCINATE:Respiratory disorder:0.42), (PROPRANOLOLPROPRANOLOL HYDROCHLORIDE:Respiratory disorder:0.45), (PYRIDOXINE:Respiratory disorder:0.4), (PYRIDOXINE HYDROCHLORIDE:Respiratory disorder:0.47), (RISEDRONATE SODIUM:Respiratory disorder:0.32), (SACCHAROMYCES CEREVISIAE:Respiratory disorder:0.09), (SALMETEROL:Respiratory disorder:0.43), (SECUKINUMAB:Respiratory disorder:1.11), (SELEXIPAG:Respiratory disorder:0.26), (SOTALOL:Respiratory disorder:1.26), (SUMATRIPTAN SUCCINATE:Respiratory disorder:0.12), (SUNITINIB MALATE:Respiratory disorder:0.06), (TERBINAFINE HYDROCHLORIDE:Respiratory disorder:0.28), (TETRACYCLINE:Respiratory disorder:0.74), (THALIDOMIDE:Respiratory disorder:0.6), (THEOPHYLLINE:Respiratory disorder:0.08), (TIGECYCLINE:Respiratory disorder:0.42), (TIMOLOL:Respiratory disorder:0.98), (TOCILIZUMAB:Respiratory disorder:0.58), (TOCOPHEROL:Respiratory disorder:0.05), (VALGANCICLOVIR HYDROCHLORIDE:Respiratory disorder:0.02), (VITAMIN A:Respiratory disorder:0.4), (CILASTATIN SODIUMIMIPENEM:Respiratory distress:0.03), (CLINDAMYCINCLINDAMYCIN PHOSPHATE:Respiratory distress:1.1), (COLCHICINE:Respiratory distress:0.01), (DEXLANSOPRAZOLE:Respiratory distress:0.02), (DILTIAZEM:Respiratory distress:0.13), (DOXYCYCLINE:Respiratory distress:0.17), (IBRUTINIB:Respiratory distress:0.34), (ILOPROST:Respiratory distress:0.31), (IMIPENEM:Respiratory distress:0.05), (IRON:Respiratory distress:0.11), (ISOSORBIDE MONONITRATE:Respiratory distress:0.21), (IVACAFTOR:Respiratory distress:0.03), (NAPROXEN:Respiratory distress:0.15), (NIFEDIPINE:Respiratory distress:0.03), (OXCARBAZEPINE:Respiratory distress:0.19), (PANTOPRAZOLE:Respiratory distress:0.27), (POTASSIUM:Respiratory distress:0.15), (SERTRALINE HYDROCHLORIDE:Respiratory distress:0.45), (TAMSULOSIN:Respiratory distress:0.04), (THALIDOMIDE:Respiratory distress:0.15), (TORSEMIDE:Respiratory distress:0.26), (VALSARTAN:Respiratory distress:0.02), (VERAPAMIL HYDROCHLORIDE:Respiratory distress:0.01), (VITAMINS:Respiratory distress:0.15), (ZONISAMIDE:Respiratory distress:0.24), (ACETAMINOPHENCODEINE PHOSPHATE:Respiratory failure:0.19), (AMIODARONE HYDROCHLORIDE:Respiratory failure:0.53), (AMITRIPTYLINE:Respiratory failure:0.05), (ANASTROZOLE:Respiratory failure:0.12), (AZTREONAM:Respiratory failure:0.14), (BENDAMUSTINE HYDROCHLORIDE:Respiratory failure:0.18), (BUPROPION HYDROCHLORIDE:Respiratory failure:0.44), (CALCIUM:Respiratory failure:0.35), (CALCIUM ACETATE:Respiratory failure:0.31), (CALCIUM CARBONATECHOLECALCIFEROL:Respiratory failure:0.44), (CANDESARTAN:Respiratory failure:0.41), (CANDESARTAN CILEXETILHYDROCHLOROTHIAZIDE:Respiratory failure:0.19), (CARFILZOMIB:Respiratory failure:0.27), (CEFTRIAXONE SODIUM:Respiratory failure:0.36), (CICLESONIDE:Respiratory failure:0.16), (CLINDAMYCINCLINDAMYCIN PHOSPHATE:Respiratory failure:1.02), (CLOZAPINE:Respiratory failure:0.32), (COLCHICINE:Respiratory failure:0.05), (COLISTIMETHATE SODIUM:Respiratory failure:0.1), (DASATINIB:Respiratory failure:0.65), (DEXLANSOPRAZOLE:Respiratory failure:0.29), (DICLOFENAC:Respiratory failure:0.22), (DICLOFENAC SODIUM:Respiratory failure:0.03), (DIVALPROEX SODIUM:Respiratory failure:0.09), (EVOLOCUMAB:Respiratory failure:0.21), (FENOFIBRATE:Respiratory failure:0.15), (FORMOTEROL FUMARATE\MOMETASONE FUROATE:Respiratory failure:0.18), (GLYCOPYRRONIUM:Respiratory failure:0.08), (IBRUTINIB:Respiratory failure:0.83), (IMATINIB MESYLATE:Respiratory failure:0.07), (INDAPAMIDEPERINDOPRIL:Respiratory failure:0.03), (INSULIN DETEMIR:Respiratory failure:0.1), (IVACAFTOR:Respiratory failure:0.17), (LABETALOLLABETALOL HYDROCHLORIDE:Respiratory failure:0.04), (LEDIPASVIRSOFOSBUVIR:Respiratory failure:0.18), (LEVALBUTEROL HYDROCHLORIDE:Respiratory failure:0.33), (LITHIUM CARBONATE:Respiratory failure:0.03), (MEPOLIZUMAB:Respiratory failure:0.23), (MOXIFLOXACIN:Respiratory failure:0.16), (NILOTINIB:Respiratory failure:0.87), (OSELTAMIVIR:Respiratory failure:0.49), (OXCARBAZEPINE:Respiratory failure:0.25), (PANTOPRAZOLE MAGNESIUM:Respiratory failure:0.24), (POSACONAZOLE:Respiratory failure:0.44), (RANOLAZINE:Respiratory failure:0.33), (SECUKINUMAB:Respiratory failure:0.24), (SOTALOL:Respiratory failure:0.02), (THALIDOMIDE:Respiratory failure:0.91), (TOCILIZUMAB:Respiratory failure:0.11), (TOPIRAMATE:Respiratory failure:0.19), (VALGANCICLOVIR HYDROCHLORIDE:Respiratory failure:0.31), (.ALPHA.1-PROTEINASE INHIBITOR HUMAN:Respiratory tract congestion:0.39), (ACETAMINOPHENBUTALBITALCAFFEINE:Respiratory tract congestion:0.41), (ANASTROZOLE:Respiratory tract congestion:0.39), (ATROPINE SULFATEDIPHENOXYLATE HYDROCHLORIDE:Respiratory tract congestion:0.02), (BUDESONIDEFORMOTEROL FUMARATE:Respiratory tract congestion:0.11), (BUMETANIDE:Respiratory tract congestion:1.05), (BUPROPION:Respiratory tract congestion:0.33), (CALCIUM ACETATE:Respiratory tract congestion:0.31), (CANDESARTAN:Respiratory tract congestion:0.25), (CANDESARTAN CILEXETILHYDROCHLOROTHIAZIDE:Respiratory tract congestion:0.1), (CARISOPRODOL:Respiratory tract congestion:0.01), (CLOTRIMAZOLE:Respiratory tract congestion:0.3), (COLCHICINE:Respiratory tract congestion:0.48), (DACLIZUMAB:Respiratory tract congestion:0.07), (DALFAMPRIDINE:Respiratory tract congestion:0.22), (DESLORATADINE:Respiratory tract congestion:0.13), (DUTASTERIDE:Respiratory tract congestion:0.04), (ELETRIPTAN HYDROBROMIDE:Respiratory tract congestion:0.37), (ENALAPRIL MALEATE:Respiratory tract congestion:0.19), (EVEROLIMUS:Respiratory tract congestion:0.11), (EVOLOCUMAB:Respiratory tract congestion:0.69), (FESOTERODINE FUMARATE:Respiratory tract congestion:0.06), (FLECAINIDE:Respiratory tract congestion:0.36), (FLUTICASONE FUROATESALMETEROL XINAFOATE:Respiratory tract congestion:0.09), (FORMOTEROL FUMARATE:Respiratory tract congestion:0.09), (GLYCOPYRRONIUM:Respiratory tract congestion:0.02), (HYDROMORPHONE:Respiratory tract congestion:0.25), (HYOSCYAMINE:Respiratory tract congestion:0.03), (ILOPROST:Respiratory tract congestion:0.15), (INDOMETHACIN:Respiratory tract congestion:0.24), (INSULIN DETEMIR:Respiratory tract congestion:0.27), (IPRATROPIUM:Respiratory tract congestion:0.4), (LABETALOLLABETALOL HYDROCHLORIDE:Respiratory tract congestion:0.04), (LATANOPROST:Respiratory tract congestion:0.17), (LERCANIDIPINE:Respiratory tract congestion:0.06), (LEVALBUTEROL HYDROCHLORIDE:Respiratory tract congestion:0.49), (LISDEXAMFETAMINE DIMESYLATE:Respiratory tract congestion:0.15), (LITHIUM CARBONATE:Respiratory tract congestion:0.06), (MEPOLIZUMAB:Respiratory tract congestion:0.29), (METHADONE HYDROCHLORIDE:Respiratory tract congestion:0.01), (METHYLPREDNISOLONE ACETATE:Respiratory tract congestion:0.01), (METRONIDAZOLE:Respiratory tract congestion:1.4), (NEBIVOLOL:Respiratory tract congestion:0.41), (NILOTINIB:Respiratory tract congestion:0.33), (NITROFURANTOIN:Respiratory tract congestion:0.28), (OXYCODONE HYDROCHLORIDE:Respiratory tract congestion:0.13), (PANTOPRAZOLE MAGNESIUM:Respiratory tract congestion:0.81), (PENICILLIN:Respiratory tract congestion:0.04), (PHENOBARBITAL:Respiratory tract congestion:0.2), (PRIMIDONE:Respiratory tract congestion:0.25), (QUETIAPINE:Respiratory tract congestion:0.19), (RANOLAZINE:Respiratory tract congestion:0.64), (RISEDRONATE SODIUM:Respiratory tract congestion:0.15), (RIZATRIPTAN BENZOATE:Respiratory tract congestion:0.09), (ROFLUMILAST:Respiratory tract congestion:0.19), (SECUKINUMAB:Respiratory tract congestion:0.13), (SODIUM OXYBATE:Respiratory tract congestion:0.62), (SUMATRIPTANSUMATRIPTAN SUCCINATE:Respiratory tract congestion:0.58), (TELMISARTAN:Respiratory tract congestion:0.22), (TERIFLUNOMIDE:Respiratory tract congestion:0.34), (TIZANIDINE HYDROCHLORIDE:Respiratory tract congestion:0.53), (TOCILIZUMAB:Respiratory tract congestion:0.2), (TOFACITINIB CITRATE:Respiratory tract congestion:0.21), (VARENICLINE TARTRATE:Respiratory tract congestion:0.91), (VITAMIN B:Respiratory tract congestion:0.48), (ZONISAMIDE:Respiratory tract congestion:0.15), (ZOPICLONE:Respiratory tract congestion:0.04), (ACETAMINOPHENBUTALBITALCAFFEINE:Respiratory tract infection:0.64), (ACLIDINIUM BROMIDE:Respiratory tract infection:0.26), (ALCOHOL:Respiratory tract infection:0.08), (AMISULPRIDE:Respiratory tract infection:0.09), (ARIPIPRAZOLE:Respiratory tract infection:0.31), (BENAZEPRILBENAZEPRIL HYDROCHLORIDE:Respiratory tract infection:0.24), (CALCITRIOL:Respiratory tract infection:0.32), (CALCIUM ACETATE:Respiratory tract infection:0.2), (CANAKINUMAB:Respiratory tract infection:0.61), (CARVEDILOL PHOSPHATE:Respiratory tract infection:0.42), (COLCHICINE:Respiratory tract infection:0.79), (DACLIZUMAB:Respiratory tract infection:0.3), (DALTEPARIN:Respiratory tract infection:0), (DASATINIB:Respiratory tract infection:1.03), (DOCETAXEL:Respiratory tract infection:0.56), (DOCUSATE:Respiratory tract infection:0.46), (DOFETILIDE:Respiratory tract infection:0.26), (DRONABINOL:Respiratory tract infection:0.27), (ELETRIPTAN HYDROBROMIDE:Respiratory tract infection:0.43), (ESZOPICLONE:Respiratory tract infection:0.56), (EVOLOCUMAB:Respiratory tract infection:1.14), (EXEMESTANE:Respiratory tract infection:0.04), (FERROUS SULFATEFERROUS SULFATE, DRIED:Respiratory tract infection:1.61), (FESOTERODINE FUMARATE:Respiratory tract infection:0.48), (FINGOLIMOD HYDROCHLORIDE:Respiratory tract infection:0.43), (FLECAINIDE:Respiratory tract infection:1.03), (FOSFOMYCIN:Respiratory tract infection:0.11), (GLYCOPYRROLATE:Respiratory tract infection:0.32), (HYDROXYUREA:Respiratory tract infection:0.05), (ILOPROST:Respiratory tract infection:0.54), (IMATINIB MESYLATE:Respiratory tract infection:0.06), (IMIPRAMINE HYDROCHLORIDE:Respiratory tract infection:0.08), (INDAPAMIDEPERINDOPRIL:Respiratory tract infection:0.96), (INDOMETHACIN:Respiratory tract infection:0.72), (IVACAFTOR:Respiratory tract infection:0.44), (LANREOTIDE ACETATE:Respiratory tract infection:0.02), (LERCANIDIPINE HYDROCHLORIDE:Respiratory tract infection:0.74), (LEVALBUTEROL:Respiratory tract infection:0.07), (LINAGLIPTIN:Respiratory tract infection:0.37), (LOVASTATIN:Respiratory tract infection:1.18), (LUMACAFTOR:Respiratory tract infection:0.34), (MEPOLIZUMAB:Respiratory tract infection:0.89), (METHADONE HYDROCHLORIDE:Respiratory tract infection:0.34), (METHENAMINE HIPPURATE:Respiratory tract infection:0.23), (METHYLPREDNISOLONE ACETATE:Respiratory tract infection:0.6), (MODAFINIL:Respiratory tract infection:0.14), (NABUMETONE:Respiratory tract infection:0.02), (NALOXONE HYDROCHLORIDEOXYCODONE HYDROCHLORIDE:Respiratory tract infection:0.84), (NILOTINIB:Respiratory tract infection:1.2), (NORTRIPTYLINE:Respiratory tract infection:0.22), (OLANZAPINE:Respiratory tract infection:1.03), (PANCRELIPASE:Respiratory tract infection:0.02), (PANTOPRAZOLE MAGNESIUM:Respiratory tract infection:1.53), (PAROXETINEPAROXETINE HYDROCHLORIDE:Respiratory tract infection:0.93), (PAZOPANIB HYDROCHLORIDE:Respiratory tract infection:0.17), (PEMETREXED DISODIUM:Respiratory tract infection:0.24), (PENTAMIDINE ISETHIONATE:Respiratory tract infection:0.01), (PONATINIB:Respiratory tract infection:0.03), (PREDNISOLONEPREDNISOLONE ACETATEPREDNISOLONE SODIUM SUCCINATE:Respiratory tract infection:0.06), (PRIMIDONE:Respiratory tract infection:0.78), (PROPRANOLOL HYDROCHLORIDE:Respiratory tract infection:0.08), (PYRAZINAMIDE:Respiratory tract infection:0.73), (PYRIDOXINE HYDROCHLORIDE:Respiratory tract infection:0.27), (ROPINIROLE HYDROCHLORIDE:Respiratory tract infection:0.26), (SALMETEROL XINAFOATE:Respiratory tract infection:0.1), (SECUKINUMAB:Respiratory tract infection:0.83), (SUMATRIPTANSUMATRIPTAN SUCCINATE:Respiratory tract infection:0.41), (TERBUTALINE SULFATE:Respiratory tract infection:0.68), (TETRACYCLINE:Respiratory tract infection:0.23), (TIMOLOL:Respiratory tract infection:0.04), (TRIAMTERENE:Respiratory tract infection:0.33), (VALGANCICLOVIR HYDROCHLORIDE:Respiratory tract infection:0.25), (VINCRISTINE SULFATE:Respiratory tract infection:0.41), (ABOBOTULINUMTOXINA:Restless legs syndrome:0.25), (ACETAMINOPHENPROPOXYPHENE NAPSYLATE:Restless legs syndrome:0.8), (ACITRETIN:Restless legs syndrome:0.35), (ALCOHOL:Restless legs syndrome:1.11), (AMILORIDE HYDROCHLORIDEHYDROCHLOROTHIAZIDE:Restless legs syndrome:1.13), (AMINO ACIDSDEXTROSEELECTROLYTES NOS:Restless legs syndrome:0.84), (AMLODIPINE BESYLATEHYDROCHLOROTHIAZIDEVALSARTAN:Restless legs syndrome:1.1), (AMLODIPINE BESYLATETELMISARTAN:Restless legs syndrome:0.17), (AMLODIPINEVALSARTAN:Restless legs syndrome:1.15), (AMMONIUM CHLORIDEDEXTROMETHORPHAN\SODIUM CITRATE:Restless legs syndrome:0.34), (AMOXICILLINCLARITHROMYCINESOMEPRAZOLE:Restless legs syndrome:0.08), (AMPICILLIN SODIUMSULBACTAM SODIUM:Restless legs syndrome:0.2), (ARIPIPRAZOLE LAUROXIL:Restless legs syndrome:0.29), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Restless legs syndrome:0.01), (ASPIRINDIHYDROXYALUMINUM AMINOACETATE ANHYDROUSMAGNESIUM CARBONATE:Restless legs syndrome:0.28), (AVANAFIL:Restless legs syndrome:0.11), (AXITINIB:Restless legs syndrome:0.68), (BALSALAZIDE DISODIUM:Restless legs syndrome:0.16), (BECLOMETHASONE:Restless legs syndrome:0.52), (BETAMETHASONECALCIPOTRIENE:Restless legs syndrome:0.7), (BETHANECHOL CHLORIDE:Restless legs syndrome:0.37), (BICALUTAMIDE:Restless legs syndrome:0.25), (BILASTINE:Restless legs syndrome:0.1), (BUDESONIDEFORMOTEROL FUMARATE:Restless legs syndrome:1.46), (BUPRENORPHINENALOXONE:Restless legs syndrome:0.23), (CABOZANTINIB S-MALATE:Restless legs syndrome:0.13), (CALCIUM CARBONATEERGOCALCIFEROLRETINOL:Restless legs syndrome:0.06), (CALCIUM\MAGNESIUM:Restless legs syndrome:1.02), (CALCIUM\MAGNESIUM\ZINC:Restless legs syndrome:1.3), (CARBOMER:Restless legs syndrome:0.03), (CEFTAZIDIME:Restless legs syndrome:0), (CHLOROTHIAZIDE:Restless legs syndrome:0.48), (CHLORPHENIRAMINEHYDROCODONE:Restless legs syndrome:0.96), (CIPROFIBRATE:Restless legs syndrome:0.56), (CLIKSTAR:Restless legs syndrome:0.31), (CLOBAZAM:Restless legs syndrome:0.63), (CLOXAZOLAM:Restless legs syndrome:0.01), (COLLAGEN:Restless legs syndrome:0.02), (CRIZOTINIB:Restless legs syndrome:0.53), (CYANOCOBALAMINFOLIC ACIDPYRIDOXINE:Restless legs syndrome:0.08), (DACLIZUMAB:Restless legs syndrome:1.22), (DANTROLENE SODIUM:Restless legs syndrome:0.44), (DEXAMETHASONE ACETATE:Restless legs syndrome:0.24), (DEXAMETHASONE SODIUM PHOSPHATE:Restless legs syndrome:0.09), (DEXPANTHENOLNIACINAMIDEPYRIDOXINE HYDROCHLORIDE\RIBOFLAVIN 5′-PHOSPHATE SODIUMTHIAMINE HYDROCHLORIDE:Restless legs syndrome:0.13), (DIFLUNISAL:Restless legs syndrome:0.25), (DIMETHYL SULFOXIDE:Restless legs syndrome:0), (DIOSMIN:Restless legs syndrome:0.02), (DIPYRONEPROMETHAZINE HYDROCHLORIDE:Restless legs syndrome:0.48), (DOLASETRON MESYLATE:Restless legs syndrome:0.58), (ECONAZOLE:Restless legs syndrome:0.85), (ELBASVIRGRAZOPREVIR:Restless legs syndrome:0), (ELOTUZUMAB:Restless legs syndrome:0.22), (ELTROMBOPAG OLAMINE:Restless legs syndrome:0.62), (ENALAPRIL MALEATEHYDROCHLOROTHIAZIDE:Restless legs syndrome:0.1), (EPHEDRINE HYDROCHLORIDEGUAIFENESIN:Restless legs syndrome:0.09), (EPIRUBICIN:Restless legs syndrome:0.38), (ETHACRYNIC ACID:Restless legs syndrome:0.1), (ETHINYL ESTRADIOLNORGESTREL:Restless legs syndrome:0.45), (FENOTEROL HYDROBROMIDE:Restless legs syndrome:0.54), (FERRIC HYDROXIDE:Restless legs syndrome:0.17), (FERRIC POLYSACCHARIDE COMPLEX:Restless legs syndrome:0.07), (FERROUS BISGLYCINATE:Restless legs syndrome:0.26), (FERROUS SULFATE ANHYDROUS:Restless legs syndrome:0.04), (FERROUS SULFATEGLYCINE:Restless legs syndrome:0.51), (FLUOXETINE HYDROCHLORIDEOLANZAPINE:Restless legs syndrome:0.01), (FONDAPARINUX SODIUM:Restless legs syndrome:0.3), (FROVATRIPTAN SUCCINATE:Restless legs syndrome:0.19), (FUSIDIC ACID:Restless legs syndrome:1.37), (GLIPIZIDEMETFORMIN HYDROCHLORIDE:Restless legs syndrome:0.28), (GLYCERIN:Restless legs syndrome:0.19), (GLYCOPYRROLATEINDACATEROL MALEATE:Restless legs syndrome:0.52), (HEPARIN CALCIUM:Restless legs syndrome:0.3), (HERBALSSAW PALMETTO:Restless legs syndrome:0.12), (HYALURONATE SODIUM:Restless legs syndrome:0.73), (HYDROCHLOROTHIAZIDETELMISARTAN:Restless legs syndrome:1.41), (HYDROCODONE BITARTRATEIBUPROFEN:Restless legs syndrome:1.04), (ILOPROST:Restless legs syndrome:1.72), (IMATINIB MESYLATE:Restless legs syndrome:1.88), (IMIGLUCERASE:Restless legs syndrome:0.21), (INFLIXIMAB-DYYB:Restless legs syndrome:0.33), (INTERFERON:Restless legs syndrome:0.59), (INTERFERON ALFA-2A:Restless legs syndrome:0.64), (INTERFERON ALFA-2B:Restless legs syndrome:0.44), (INTERFERON GAMMA-1B:Restless legs syndrome:0.4), (IRON SUCROSE:Restless legs syndrome:0.86), (IVABRADINE:Restless legs syndrome:0.15), (LANREOTIDE ACETATE:Restless legs syndrome:0.3), (LAPATINIB DITOSYLATE:Restless legs syndrome:0.45), (LECITHIN:Restless legs syndrome:0.12), (LERCANIDIPINE:Restless legs syndrome:2.03), (LOMITAPIDE MESYLATE:Restless legs syndrome:1.26), (LUMACAFTOR:Restless legs syndrome:0), (MEPERIDINE:Restless legs syndrome:0.56), (MEPOLIZUMAB:Restless legs syndrome:1.45), (METFORMIN HYDROCHLORIDEROSIGLITAZONE MALEATE:Restless legs syndrome:0.03), (METHENAMINE:Restless legs syndrome:0.09), (METHENAMINE MANDELATE:Restless legs syndrome:0.14), (METRELEPTIN:Restless legs syndrome:0.07), (MEXILETINE:Restless legs syndrome:0.08), (MEXILETINE HYDROCHLORIDE:Restless legs syndrome:0.07), (MIGLUSTAT:Restless legs syndrome:0.03), (MOEXIPRIL HYDROCHLORIDE:Restless legs syndrome:0.39), (NEFAZODONE HYDROCHLORIDE:Restless legs syndrome:0.04), (NICARDIPINE:Restless legs syndrome:0.34), (NOREPINEPHRINE BITARTRATE:Restless legs syndrome:0.16), (OXYBUTYNINOXYBUTYNIN CHLORIDE:Restless legs syndrome:0.54), (PAZOPANIB HYDROCHLORIDE:Restless legs syndrome:1.87), (PEMETREXED DISODIUM:Restless legs syndrome:1.07), (PENICILLIN G:Restless legs syndrome:0.48), (PENICILLIN V:Restless legs syndrome:0.35), (PERPHENAZINE:Restless legs syndrome:0.31), (PILSICAINIDE HYDROCHLORIDE:Restless legs syndrome:0.11), (PONATINIB:Restless legs syndrome:1.87), (PRAMLINTIDE ACETATE:Restless legs syndrome:0.09), (PREDNISOLONE SODIUM PHOSPHATE:Restless legs syndrome:0.01), (PROTRIPTYLINE HYDROCHLORIDE:Restless legs syndrome:0.83), (PYRAZINAMIDE:Restless legs syndrome:0.75), (QUINACRINE:Restless legs syndrome:0.09), (RETINOL:Restless legs syndrome:0.74), (RIOCIGUAT:Restless legs syndrome:1.05), (SALMETEROL:Restless legs syndrome:0.5), (SELEGILINE:Restless legs syndrome:1.01), (SELEGILINE HYDROCHLORIDE:Restless legs syndrome:0.55), (SIBUTRAMINE HYDROCHLORIDE:Restless legs syndrome:0.15), (SIPULEUCEL-T:Restless legs syndrome:0.18), (SODIUM PHOSPHATE, DIBASIC\SODIUM PHOSPHATE, MONOBASIC:Restless legs syndrome:0.8), (SODIUM POLYSTYRENE SULFONATE:Restless legs syndrome:0.19), (STARCH, WHEAT:Restless legs syndrome:0.41), (SUFENTANIL CITRATE:Restless legs syndrome:0.1), (SULFAMETHOXAZOLE:Restless legs syndrome:1.37), (TAFLUPROST:Restless legs syndrome:0.08), (TAMOXIFEN CITRATE:Restless legs syndrome:0.04), (TEDUGLUTIDE\WATER:Restless legs syndrome:0.71), (TELOTRISTAT ETHYL:Restless legs syndrome:0.12), (TERBUTALINE:Restless legs syndrome:0.31), (TESTOSTERONE UNDECANOATE:Restless legs syndrome:0.35), (TETRACYCLINE:Restless legs syndrome:1.66), (TIBOLONE:Restless legs syndrome:0.08), (TINZAPARIN:Restless legs syndrome:0.27), (TINZAPARIN SODIUM:Restless legs syndrome:0.77), (TRIMIPRAMINE MALEATE:Restless legs syndrome:0.73), (TRIPTORELIN PAMOATE:Restless legs syndrome:0.12), (ACARBOSE:Restlessness:0.15), (ACETAMINOPHENOXYCODONEOXYC ODONE HYDROCHLORIDE:Restlessness:0.11), (ALCOHOL:Restlessness:1.01), (ALENDRONIC ACID:Restlessness:0.3), (ARMODAFINIL:Restlessness:0.7), (BRINZOLAMIDE:Restlessness:0.08), (CALCIUM CARBONATECHOLECALCIFEROL:Restlessness:1.43), (CALCIUM CITRATE:Restlessness:0.38), (CANDESARTAN CILEXETILHYDROCHLOROTHIAZIDE:Restlessness:0.64), (CANNABIS SATIVA SUBSP. INDICA TOP:Restlessness:0.2), (CARVEDILOL PHOSPHATE:Restlessness:0.21), (CEFAZOLIN SODIUM:Restlessness:0.12), (CEFOTAXIME SODIUM:Restlessness:0.07), (CERTOLIZUMAB PEGOL:Restlessness:1.2), (CHOLESTYRAMINE:Restlessness:0.39), (DACLIZUMAB:Restless ness:0.26), (DASATINIB:Restlessness:0.66), (DESLORATADINE:Restlessness:0.48), (DOXAZ OSIN MESYLATE:Restlessness:0.42), (DOXAZOSINDOXAZOSIN MESYLATE:Restlessness:0.45), (DRONABINOL:Restlessness:0.12), (EDOXABAN TOSYLATE:Restlessness:0.43), (ENALAPRIL MALEATE:Restlessness:0.64), (ESTROGENS, CONJUGATEDMEDROXYPROGESTERONE ACETATE:Restlessness:0.39), (ETORICOXIB:Restlessness:0.24), (EVOLOCUMAB:Restlessne ss:0.59), (FESOTERODINE FUMARATE:Restlessness:0.67), (FLUDROCORTISONE ACETATE:Restlessness:0.15), (FLUOXETINEFLUOXETINE HYDROCHLORIDE:Restlessness:0.93), (FLUPHENAZINE:Restlessness:0.08), (FLUTICASON E FUROATESALMETEROL XINAFOATE:Restlessness:0.32), (FLUTICASONE FUROATEVILANTEROL TRIFENATATE:Restlessness:0.18), (FOSPHENYTOINFOSPHENYTOIN SODIUM:Restlessness:0.01), (GLIPIZIDE:Restlessness:0.61), (HYALURONATE SODIUM:Restlessness:0.06), (HYDROCHLOROTHIAZIDEIRBESARTAN:Restlessness:0.39), (HYDROXYCHLOROQUINE DIPHOSPHATE:Restlessness:0.15), (ILOPROST:Restlessness:0.36), (IMATINIB MESYLATE:Restlessness:0.83), (INDAPAMIDE:Restlessness:0.5), (INDAPAMIDEPERINDO PRIL:Restlessness:0.26), (IPRATROPIUM:Restlessness:0.62), (LEFLUNOMIDE:Restlessness:0.04), (LERCANIDIPINE:Restlessness:0.71), (LERCANIDIPINE HYDROCHLORIDE:Restlessness:0.23), (LEVALBUTEROL HYDROCHLORIDE:Restlessness:0.35), (METHYLPREDNISOLONE ACETATE:Restlessness:0.4), (MIDODRINE:Restlessness:0.25), (MOXIFLOXACIN:Restlessnes s:0.11), (NIACIN:Restlessness:0.19), (NICOTINE:Restlessness:0.39), (OXCARBAZEPINE:Restl essness:2.49), (PEMETREXED DISODIUM:Restlessness:0.19), (PENICILLIN:Restlessness:0.32), (PERAMPANEL:Restlessness: 0.08), (PITAVASTATIN CALCIUM:Restlessness:0.42), (PRAZOSIN:Restlessness:0.31), (PROCYCLIDINE:Restlessness: 0.02), (RABEPRAZOLE:Restlessness:0.36), (RIZATRIPTAN BENZOATE:Restlessness:0.28), (ROFLUMILAST:Restlessness:0.34), (SACUBITRILVALSAR TAN:Restlessness:0.81), (SELEGILINE:Restlessness:0.24), (SELEGILINE HYDROCHLORIDE:Restlessness:0.07), (SITAGLIPTIN:Restlessness:0.03), (SOLIFENACIN SUCCINATE:Restlessness:0.36), (SOTALOL:Restlessness:0.7), (SUCRALFATE:Restlessness:0.47), (SULFAMETHOXAZOLE:Restlessness:0.67), (SUMATRIPTAN:Restlessness:0.12), (SUM ATRIPTAN SUCCINATE:Restlessness:0.17), (SUMATRIPTANSUMATRIPTAN SUCCINATE:Restlessness:0.21), (TESTOSTERONE:Restlessness:0.06), (THALIDOMIDE:Rest lessness:1.06), (TINZAPARIN SODIUM:Restlessness:0.22), (TRIHEXYPHENIDYL:Restlessness:0.57), (VALGANCICLOVIR HYDROCHLORIDE:Restlessness:0.21), (VARENICLINE TARTRATE:Restlessness:1.58), (ZUCLOPENTHIXOL DECANOATE:Restlessness:0.19), (POLYMYXIN B SULFATETRIMETHOPRIM SULFATE:Retinal oedema:0.26), (CALCITRIOL:Retinal vein occlusion:0.25), (CALCIUM CARBONATECHOLECALCIFEROL:Retinal vein occlusion:0.12), (CANDESARTAN CILEXETILHYDROCHLOROTHIAZIDE:Retinal vein occlusion:0.15), (CARTEOLOL HYDROCHLORIDE:Retinal vein occlusion:0.38), (CERTOLIZUMAB PEGOL:Retinal vein occlusion:0.48), (DEXLANSOPRAZOLE:Retinal vein occlusion:0.28), (DOCUSATE SODIUM:Retinal vein occlusion:0.17), (ENOXAPARIN:Retinal vein occlusion:0.16), (EVEROLIMUS:Retinal vein occlusion:0.03), (EVOLOCUMAB:Retinal vein occlusion:0.99), (FERROUS SULFATEFERROUS SULFATE, DRIED:Retinal vein occlusion:0.49), (LEDIPASVIRSOFOSBUVIR:Retinal vein occlusion:0), (NEBIVOLOL:Retinal vein occlusion:0.17), (POLYMYXIN B SULFATETRIMETHOPRIM SULFATE:Retinal vein occlusion:0.13), (POVIDONE:Retinal vein occlusion:0.5), (RANOLAZINE:Retinal vein occlusion:0.69), (SECUKINUMAB:Retinal vein occlusion:0.1), (SOTALOL:Retinal vein occlusion:0.09), (TIMOLOLTRAVOPROST:Retinal vein occlusion:0.23), (TOFACITINIB CITRATE:Retinal vein occlusion:0.03), (TOPIRAMATE:Retinal vein occlusion:0.25), (TRIAMTERENE:Retinal vein occlusion:0.3), (ANASTROZOLE:Rheumatic disorder:1.05), (BENAZEPRIL HYDROCHLORIDE:Rheumatic disorder:0.21), (CALCIUM CITRATE:Rheumatic disorder:0.03), (CARFILZOMIB:Rheumatic disorder:0.02), (CHOLESTYRAMINE:Rheumatic disorder:0.51), (CLOTRIMAZOLE:Rheumatic disorder:0.06), (CLOZAPINE:Rheumatic disorder:0.42), (DALFAMPRIDINE:Rheumatic disorder:0.51), (DASATINIB:Rheumatic disorder:0.43), (DEVICE:Rheumatic disorder:0.25), (DICYCLOMINE HYDROCHLORIDE:Rheumatic disorder:0.09), (DOXAZOSINDOXAZOSIN MESYLATE:Rheumatic disorder:0.09), (DUTASTERIDE:Rheumatic disorder:0.29), (EVOLOCUMAB:Rheumatic disorder:1.04), (EZETIMIBE:Rheumatic disorder:0.5), (EZETIMIBESIMVASTATIN:Rheumatic disorder:0.05), (FESOTERODINE FUMARATE:Rheumatic disorder:0.06), (FLUTICASONE:Rheumatic disorder:0.01), (FULVESTRANT:Rheumatic disorder:0.03), (GLIPIZIDE:Rheumatic disorder:0.42), (GLYCOPYRRONIUMINDACATEROL:Rheumatic disorder:0.03), (HEPARIN SODIUM:Rheumatic disorder:0.31), (HYDRALAZINE HYDROCHLORIDE:Rheumatic disorder:0.08), (HYDROCHLOROTHIAZIDEIRBESARTAN:Rheumatic disorder:0.4), (HYDROXYCHLOROQUINE DIPHOSPHATE:Rheumatic disorder:0.46), (HYDROXYUREA:Rheumatic disorder:0.31), (INDAPAMIDEPERINDOPRIL:Rheumatic disorder:0.47), (IRON:Rheumatic disorder:0.62), (ISOSORBIDE:Rheumatic disorder:0.14), (LEDIPASVIRSOFOSBUVIR:Rheumatic disorder:0.48), (LERCANIDIPINE:Rheumatic disorder:0.75), (LITHIUM CARBONATE:Rheumatic disorder:0.64), (MELATONIN:Rheumatic disorder:0.11), (METHIMAZOLE:Rheumatic disorder:0), (METHYLPREDNISOLONE ACETATE:Rheumatic disorder:0.28), (METHYLPREDNISOLONE SODIUM SUCCINATE:Rheumatic disorder:0.85), (METRONIDAZOLE:Rheumatic disorder:1.18), (NYSTATIN:Rheumatic disorder:0.07), (OMEGA-3-ACID ETHYL ESTERS:Rheumatic disorder:0.11), (OXCARBAZEPINE:Rheumatic disorder:0.54), (OXYGEN:Rheumatic disorder:0.49), (PANTOPRAZOLE MAGNESIUM:Rheumatic disorder:0.56), (PAROXETINE:Rheumatic disorder:0.3), (PRIMIDONE:Rheumatic disorder:0.12), (PROBIOTICS NOS:Rheumatic disorder:0.22), (PYRIDOXINE HYDROCHLORIDE:Rheumatic disorder:0.03), (RANOLAZINE:Rheumatic disorder:0.51), (SELEXIPAG:Rheumatic disorder:0.22), (TIZANIDINE HYDROCHLORIDE:Rheumatic disorder:0.19), (TRIAMCINOLONE ACETONIDE:Rheumatic disorder:0.03), (TRIAMTERENE:Rheumatic disorder:0.07), (VANCOMYCIN:Rheumatic disorder:0.05), (.ALPHA.-LIPOIC ACID, (+/−)-:Rheumatoid arthritis:0.01), (.ALPHA.-TOCOPHEROL ACETATE, D-ASCORBIC ACIDCALCIUM PANTOTHENATEFOLIC ACIDMANGANESENIACINAMIDEPYRIDOXINE\RIBOFLAVINTHIAMINEVITAMIN A\ZINC OXIDE:Rheumatoid arthritis:0.35), (.ALPHA.-TOCOPHEROL ACETATE, DL-.BETA.-CAROTENEASCORBIC ACIDCALCIUM CARBONATECALCIUM PANTOTHENATECHOLECALCIFEROLCYANOCOBALAMINDOCUSATE SODIUMFERROUS FUMARATEFOLIC ACIDNIACINAMIDEPYRIDOXINE HYDROCHLORIDE\RIBOFLAVINTHIAMINE MONONITRATE\ZINC OXIDE:Rheumatoid arthritis:0.56), (.ALPHA.-TOCOPHEROL SUCCINATE, D-ASCORBIC ACIDBIOTINCALCIUM PANTOTHENATECOBALAMINFOLIC ACIDNIACINAMIDEPYRIDOXINE HYDROCHLORIDE\RIBOFLAVINSELENOCYSTEINETHIAMINE MONONITRATE\ZINC CITRATE:Rheumatoid arthritis:1.23), (.ALPHA.-TOCOPHEROL.ALPHA.-TOCOPHEROL ACETATE.ALPHA.-TOCOPHEROL ACETATE, DL-ASCORBIC ACIDCHOLECALCIFEROLCOBALAMINCYANOCOBALAMINFLUORIDE IONFOLIC ACIDNIACINNIACINAMIDEPYRIDOXINEPYRIDOXINE HYDROCHLORIDE\RIBOFLAVIN\SODIUM ASCORBATE\SODIUM FLUORIDETHIAMINETHIAMINE MONONITRATEVITAMIN AVITA:Rheumatoid arthritis:0.08), (.BETA.-HYDROXYISOVALERIC ACIDARGININEGLUTAMINE:Rheumatoid arthritis:1.02), (.GAMMA.-AMINOBUTYRIC ACID:Rheumatoid arthritis:0.09), (ABACAVIR SULFATEDOLUTEGRAVIR SODIUMLAMIVUDINE:Rheumatoid arthritis:0.44), (ACAMPROSATE:Rheumatoid arthritis:0.8), (ACAMPROSATE CALCIUM:Rheumatoid arthritis:0.06), (ACETAMINOPHEN\ASPIRIN:Rheumatoid arthritis:0.25), (ACETAMINOPHEN\ASPIRINSALICYLAMIDE:Rheumatoid arthritis:0.32), (ACETAMINOPHEN\CAFFEINEPROPOXYPHENE:Rheumatoid arthritis:0.14), (ACETAMINOPHENCHLORPHENIRAMINE MALEATEDEXTROMETHORPHAN HYDROBROMIDEPSEUDOEPHEDRINE HYDROCHLORIDE:Rheumatoid arthritis:0.09), (ACETAMINOPHENCHLORPHENIRAMINE MALEATEPSEUDOEPHEDRINE HYDROCHLORIDE:Rheumatoid arthritis:0.7), (ACETAMINOPHENDIPHENHYDRAMINEDIPHENHYDRAMINE CITRATE:Rheumatoid arthritis:0.12), (ACETAMINOPHENGUAIFENESINPHENYLEPHRINE HYDROCHLORIDE:Rheumatoid arthritis:0.21), (ACETAMINOPHENPHENYLEPHRINE:Rheumatoid arthritis:0.04), (ACETAMINOPHENPSEUDOEPHEDRINE HYDROCHLORIDE:Rheumatoid arthritis:0.25), (ACLIDINIUM BROMIDEFORMOTEROL FUMARATE:Rheumatoid arthritis:0.83), (ACOTIAMIDE HYDROCHLORIDE:Rheumatoid arthritis:0.03), (ADENINE:Rheumatoid arthritis:0.45), (ADUCANUMAB:Rheumatoid arthritis:0.19), (ALBUTEROLGUAIFENESIN:Rheumatoid arthritis:0.63), (ALGELDRATE:Rheumatoid arthritis:0.15), (ALGINIC ACIDALUMINUM HYDROXIDEMAGNESIUM CARBONATE:Rheumatoid arthritis:0.23), (ALISKIRENHYDROCHLOROTHIAZIDE:Rheumatoid arthritis:0.93), (ALOSETRON:Rheumatoid arthritis:0.02), (ALPHA-TOCOPHEROLASCORBIC ACIDCHOLECALCIFEROLCOPPERCYANOCOBALAMINFOLIC ACIDLUTEINOMEGA-3 FATTY ACIDSPYRIDOXINE HYDROCHLORIDETAURINEVITAMIN A PALMITATEZEAXANTHINZINC PICOLINATE:Rheumatoid arthritis:0.29), (ALPROSTADILPAPAVERINEPHENTOLAMINE:Rheumatoid arthritis:0.39), (ALUMINUM HYDROXIDEMAGNESIUM OXIDEOXETHAZAINE:Rheumatoid arthritis:0.34), (ALUMINUM SILICATE:Rheumatoid arthritis:0.57), (AMANTADINE SULFATE:Rheumatoid arthritis:0.48), (AMDINOCILLIN PIVOXIL HYDROCHLORIDE:Rheumatoid arthritis:0.45), (AMINO ACIDSCARBOHYDRATESMINERALSSOYBEAN OILVITAMINS:Rheumatoid arthritis:1.08), (AMINO ACIDSDEXTROSEELECTROLYTES NOS:Rheumatoid arthritis:1.21), (AMINOSALICYLIC ACID:Rheumatoid arthritis:0.91), (AMITRIPTYLINEPERPHENAZINE:Rheumatoid arthritis:0.28), (AMLODIPINE BESYLATEATORVASTATIN CALCIUM TRIHYDRATE:Rheumatoid arthritis:0.12), (AMMONIUM CHLORIDEDEXTROMETHORPHAN\SODIUM CITRATE:Rheumatoid arthritis:1.92), (AMMONIUM GLYCYRRHIZATECYSTEINE HYDROCHLORIDEGLYCINE:Rheumatoid arthritis:0.4), (AMOBARBITAL SODIUM:Rheumatoid arthritis:0.48), (AMOROLFINE:Rheumatoid arthritis:0.22), (AMOXICILLINCLARITHROMYCINLANSOPRAZOLE:Rheumatoid arthritis:1.09), (ANAGRELIDE:Rheumatoid arthritis:1.04), (APOMORPHINE:Rheumatoid arthritis:0.15), (APRINDINE HYDROCHLORIDE:Rheumatoid arthritis:0.87), (ARCTOSTAPHYLOS UVA-URSI LEAFURTICA DIOICA:Rheumatoid arthritis:0.52), (ARGATROBAN:Rheumatoid arthritis:0.79), (ARIPIPRAZOLE LAUROXIL:Rheumatoid arthritis:1.25), (ARNICA MONTANA:Rheumatoid arthritis:0.15), (ARTEMETHERLUMEFANTRINE:Rheumatoid arthritis:0.04), (ASCORBIC ACIDBIOTINCYANOCOBALAMINFOLIC ACIDNIACINPANTOTHENIC ACIDPYRIDOXINE\RIBOFLAVINTHIAMINE:Rheumatoid arthritis:1.52), (ASCORBIC ACIDCHROMIUMSELENIUMVANADYL SULFATE:Rheumatoid arthritis:0.06), (ASPIRIN\CAFFEINESALICYLAMIDE:Rheumatoid arthritis:1.1), (ATOSIBAN:Rheumatoid arthritis:0.06), (ATOVAQUONEPROGUANIL:Rheumatoid arthritis:0.16), (AVOBENZONEHOMOSALATEOCTISALATE:Rheumatoid arthritis:0.62), (AXITINIB:Rheumatoid arthritis:1.98), (BACILLUS CALMETTE-GUERIN SUBSTRAIN CONNAUGHT LIVE ANTIGEN:Rheumatoid arthritis:0.5), (BACITRACIN ZINC:Rheumatoid arthritis:0.06), (BEE POLLEN:Rheumatoid arthritis:0.77), (BELATACEPT:Rheumatoid arthritis:0.96), (BELLADONNAERGOTAMINE TARTRATEPHENOBARBITAL:Rheumatoid arthritis:0.01), (BENFLUOREX:Rheumatoid arthritis:0.33), (BENZETHONIUM CHLORIDE:Rheumatoid arthritis:0), (BEPOTASTINEBEPOTASTINE BESILATE:Rheumatoid arthritis:0.41), (BERBERINE:Rheumatoid arthritis:0.3), (BETA GLUCAN:Rheumatoid arthritis:0.47), (BETAINE:Rheumatoid arthritis:0.1), (BETAMETHASONE BUTYRATE PROPIONATE:Rheumatoid arthritis:1.03), (BETAMETHASONE DIPROPIONATECLOTRIMAZOLEGENTAMICIN SULFATE:Rheumatoid arthritis:0.45), (BETAMETHASONE DIPROPIONATEGENTAMICIN SULFATE:Rheumatoid arthritis:1.34), (BETAMIPRONPANIPENEM:Rheumatoid arthritis:0.2), (BEXAROTENE:Rheumatoid arthritis:0.08), (BICARBONATE IONPOLYETHYLENE GLYCOLSPOTASSIUM CATION\SODIUM CHLORIDE\SODIUM SULFATE ANHYDROUS:Rheumatoid arthritis:0.05), (LONGUM:Rheumatoid arthritis:1.12), (BISACODYL OR GLYCERIN OR MINERAL OIL OR SODIUM PHOSPHATE, DIBASIC\SODIUM PHOSPHATE, MONOBASIC:Rheumatoid arthritis:0.17), (BISMUTH SUBCITRATE POTASSIUMMETRONIDAZOLETETRACYCLINE HYDROCHLORIDE:Rheumatoid arthritis:0.51), (BIVALIRUDIN:Rheumatoid arthritis:0.03), (BLONANSERIN:Rheumatoid arthritis:0.75), (BORNAPRINE HYDROCHLORIDE:Rheumatoid arthritis:0.11), (BOSUTINIB MONOHYDRATE:Rheumatoid arthritis:0.63), (BRENTUXIMAB VEDOTIN:Rheumatoid arthritis:0.26), (BRIMONIDINE TARTRATEDORZOLAMIDE HYDROCHLORIDETIMOLOL MALEATE:Rheumatoid arthritis:1.39), (BRODALUMAB:Rheumatoid arthritis:0.54), (BROMFENACBROMFENAC SODIUM:Rheumatoid arthritis:0.01), (BROMPHENIRAMINE MALEATEDEXTROMETHORPHAN HYDROBROMIDEPSEUDOEPHEDRINE HYDROCHLORIDE:Rheumatoid arthritis:0.25), (BUDESONIDEINDACATEROL MALEATE:Rheumatoid arthritis:0.67), (BUFORMIN:Rheumatoid arthritis:0.5), (CABAZITAXEL:Rheumatoid arthritis:0.65), (CABOZANTINIB:Rheumatoid arthritis:0.59), (CABOZANTINIB S-MALATE:Rheumatoid arthritis:1.02), (CADEXOMER IODINE:Rheumatoid arthritis:0.15), (CAFFEINEMAGNESIUM SALICYLATE:Rheumatoid arthritis:0.65), (CAFFEINEPHENOBARBITAL:Rheumatoid arthritis:0.33), (CAJUPUT OILEUCALYPTUS OILPEPPERMINT OIL:Rheumatoid arthritis:1.19), (CALCIUM CARBIMIDE:Rheumatoid arthritis:1.02), (CALCIUM CARBONATEERGOCALCIFEROL:Rheumatoid arthritis:1.17), (CALCIUM CARBONATEMAGNESIUM HYDROXIDE:Rheumatoid arthritis:1.39), (CALCIUM CHLORIDEDEXTROSE\POTASSIUM CHLORIDE\SODIUM ACETATE\SODIUM CHLORIDE:Rheumatoid arthritis:0.69), (CALCIUM GLYCEROPHOSPHATE:Rheumatoid arthritis:0.92), (CALCIUM PANTOTHENATENIACINAMIDEPYRIDOXINE HYDROCHLORIDE\RIBOFLAVINTHIAMINE HYDROCHLORIDE:Rheumatoid arthritis:0.26), (CALCIUM PHOSPHATE, DIBASIC, ANHYDROUSFERROUS SUCCINATEMAGNESIUM HYDROXIDE:Rheumatoid arthritis:0.52), (CAMPHOR (NATURAL)MENTHOLPRAMOXINE HYDROCHLORIDE:Rheumatoid arthritis:0.45), (CAMPHOR (SYNTHETIC)CAPSICUM OLEORESINMENTHOLMETHYL SALICYLATE:Rheumatoid arthritis:0.16), (CAMPHORCAPSICUMMETHYL SALICYLATE:Rheumatoid arthritis:0.23), (CAMPHORMENTHOLMETHYL SALICYLATE:Rheumatoid arthritis:1.13), (CANDESARTAN CILEXETILFELODIPINE:Rheumatoid arthritis:0.29), (CAPSICUM:Rheumatoid arthritis:0.44), (CAPSICUM OLEORESIN:Rheumatoid arthritis:0.02), (CARBINOXAMINE MALEATE:Rheumatoid arthritis:0.73), (CARBOHYDRATESPOTASSIUM CHLORIDE\SODIUM CHLORIDE\SODIUM LACTATE:Rheumatoid arthritis:0.38), (CARBOXYMETHYLCELLULOSE SODIUMGELATINPECTIN:Rheumatoid arthritis:0.23), (CARBOXYMETHYLCELLULOSE SODIUMHYPROMELLOSES:Rheumatoid arthritis:0.26), (CARBOXYMETHYLCELLULOSE SODIUMSORBINICATE:Rheumatoid arthritis:0.54), (CARIPRAZINE:Rheumatoid arthritis:0.18), (CARMUSTINE:Rheumatoid arthritis:0.78), (CARNITINE:Rheumatoid arthritis:0.59), (CARPERITIDE:Rheumatoid arthritis:0.65), (CEFOPERAZONE SODIUMSULBACTAM SODIUM:Rheumatoid arthritis:0.06), (CEFTAZIDIME SODIUM:Rheumatoid arthritis:0.97), (CEFTERAM PIVOXIL:Rheumatoid arthritis:1), (CEPHALOSPORIN C:Rheumatoid arthritis:0.16), (CEPHARANTHINE:Rheumatoid arthritis:0.6), (CERIVASTATIN:Rheumatoid arthritis:0.03), (CERIVASTATIN SODIUM:Rheumatoid arthritis:3.03), (CHAMOMILE:Rheumatoid arthritis:0.63), (CHIMAPHILA UMBELLATAMANGANESE CHLORIDEPOPULUS TREMULOIDESSODIUM TAUROCHOLATE:Rheumatoid arthritis:0.29), (CHLOPHEDIANOL HYDROCHLORIDEDEXBROMPHENIRAMINE MALEATEPHENYLEPHRINE HYDROCHLORIDE:Rheumatoid arthritis:0.05), (CHLORDIAZEPOXIDECLIDINIUM:Rheumatoid arthritis:0.47), (CHLORIDE ION:Rheumatoid arthritis:0.12), (CHLORMADINONE ACETATEMESTRANOL:Rheumatoid arthritis:0.05), (CHLORPHENIRAMINE MALEATEHYDROCODONE BITARTRATEPSEUDOEPHEDRINE HYDROCHLORIDE:Rheumatoid arthritis:0.07), (CHOLINE ALFOSCERATE:Rheumatoid arthritis:0.72), (CICLOPIROX OLAMINE:Rheumatoid arthritis:1.06), (CIMETIDINEFAMOTIDINERANITIDINE HYDROCHLORIDE:Rheumatoid arthritis:0.6), (CINNAMON OIL:Rheumatoid arthritis:0.06), (CIPROFLOXACIN HYDROCHLORIDEHYDROCORTISONE:Rheumatoid arthritis:0.68), (CITICOLINE SODIUM:Rheumatoid arthritis:0.39), (CITRIC ACID MONOHYDRATETRISODIUM CITRATE DIHYDRATE:Rheumatoid arthritis:0.23), (CLOBETASONE BUTYRATENYSTATINOXYTETRACYCLINE CALCIUM:Rheumatoid arthritis:0.01), (CLOFAZIMINE:Rheumatoid arthritis:0.43), (CLOMETHIAZOLE:Rheumatoid arthritis:0.21), (CLOPERASTINE:Rheumatoid arthritis:0.56), (CLOSTRIDIUM TETANICLOSTRIDIUM TETANI TOXOID ANTIGEN (FORMALDEHYDE INACTIVATED)DIPHTHERIAECORYNEBACTERIUM DIPHTHERIAE TOXOID ANTIGEN (FORMALDEHYDE INACTIVATED):Rheumatoid arthritis:0.59), (COAGULATION FACTOR IX RECOMBINANT HUMANISOPROPYL ALCOHOL:Rheumatoid arthritis:0.58), (CODEINE PHOSPHATEGUAIFENESINPSEUDOEPHEDRINE HYDROCHLORIDE:Rheumatoid arthritis:0.79), (CODEINE PHOSPHATEPROMETHAZINE:Rheumatoid arthritis:1.04), (COLD CREAM:Rheumatoid arthritis:0.02), (COLESTILAN CHLORIDE:Rheumatoid arthritis:1.24), (COUMARIN:Rheumatoid arthritis:0.52), (CRATAEGUS LAEVIGATA FRUIT:Rheumatoid arthritis:0.29), (CREATINE MONOHYDRATE:Rheumatoid arthritis:0.73), (CREATININE:Rheumatoid arthritis:0.23), (CROFELEMER:Rheumatoid arthritis:0.01), (CROTALIDAE POLYVALENT IMMUNE FAB (OVINE):Rheumatoid arthritis:0.33), (CYCLOPENTOLATE:Rheumatoid arthritis:0.83), (CYPROTERONE:Rheumatoid arthritis:0.9), (CYPROTERONE ACETATE:Rheumatoid arthritis:1.34), (DACLATASVIR DIHYDROCHLORIDE:Rheumatoid arthritis:0.16), (DASABUVIR SODIUM MONOHYDRATEOMBITASVIR HEMINONAHYDRATEPARITAPREVIR DIHYDRATERITONAVIR:Rheumatoid arthritis:0.17), (DEFERIPRONE:Rheumatoid arthritis:0.74), (DEFEROXAMINE MESYLATE:Rheumatoid arthritis:0.59), (DEPRODONE PROPIONATE:Rheumatoid arthritis:0.61), (DEXAMETHASONEFRAMYCETIN SULFATEGRAMICIDIN:Rheumatoid arthritis:0.34), (DEXAMETHASONENEOMYCINPOLYMYXIN B SULFATE:Rheumatoid arthritis:0.22), (DEXTRAN SULFATE SODIUM SALT:Rheumatoid arthritis:1.26), (DIACETYL BENZOYL LATHYROL:Rheumatoid arthritis:0.16), (DIASTASE:Rheumatoid arthritis:0.79), (DIBUCAINE:Rheumatoid arthritis:0.1), (DIBUCAINE HYDROCHLORIDEHYDROCORTISONE:Rheumatoid arthritis:0.5), (DICHLORPHENAMIDE:Rheumatoid arthritis:0.41), (DIHYDROERGOCRISTINE MESYLATE:Rheumatoid arthritis:0.07), (DIMETHYL SULFONEGLUCOSAMINE SULFATE:Rheumatoid arthritis:0.28), (DIPHENHYDRAMINE HYDROCHLORIDEPHENYLEPHRINE HYDROCHLORIDE:Rheumatoid arthritis:1.37), (DISOPYRAMIDE PHOSPHATE:Rheumatoid arthritis:1), (DOLASETRON:Rheumatoid arthritis:1.04), (DOLASETRON MESYLATE:Rheumatoid arthritis:1.39), (DORIPENEM MONOHYDRATE:Rheumatoid arthritis:0.02), (DORNASE ALFA:Rheumatoid arthritis:0.14), (DROPERIDOL:Rheumatoid arthritis:0.5), (ECALLANTIDE:Rheumatoid arthritis:0.34), (EDARAVONE:Rheumatoid arthritis:1.22), (EDETIC ACID:Rheumatoid arthritis:0.49), (ELBASVIRGRAZOPREVIR:Rheumatoid arthritis:0.38), (ELIGLUSTAT:Rheumatoid arthritis:0.2), (ELM:Rheumatoid arthritis:0.23), (ELOTUZUMAB:Rheumatoid arthritis:1.42), (EMTRICITABINERILPIVIRINE HYDROCHLORIDETENOFOVIR DISOPROXIL FUMARATE:Rheumatoid arthritis:0.23), (EMTRICITABINERILPIVIRINETENOFOVIR DISOPROXIL FUMARATE:Rheumatoid arthritis:0.23), (EPHEDRINE HYDROCHLORIDEGUAIFENESIN:Rheumatoid arthritis:0.45), (EPIRUBICIN HYDROCHLORIDE:Rheumatoid arthritis:0.09), (ESCHERICHIA COLI:Rheumatoid arthritis:0.51), (ESMOLOL HYDROCHLORIDE:Rheumatoid arthritis:0.69), (ESSENTIAL OILS:Rheumatoid arthritis:0.61), (ESTRADIOL BENZOATE:Rheumatoid arthritis:1.98), (ETHINYL ESTRADIOLFERROUS FUMARATENORETHINDRONE:Rheumatoid arthritis:0.48), (ETILEFRINE HYDROCHLORIDE:Rheumatoid arthritis:0.3), (ETRETINATE:Rheumatoid arthritis:2.81), (EZETIMIBEROSUVASTATIN:Rheumatoid arthritis:0.08), (FENOPROFEN:Rheumatoid arthritis:0.69), (FENUGREEK SEED:Rheumatoid arthritis:0.36), (FERRIC CITRATE ANHYDROUS:Rheumatoid arthritis:1.13), (FERRITIN:Rheumatoid arthritis:0.17), (FERROUS ASPARTO GLYCINATEFERROUS FUMARATE:Rheumatoid arthritis:0.15), (FEVERFEW:Rheumatoid arthritis:0.42), (FILGRASTIM-SNDZ:Rheumatoid arthritis:0.46), (FLOPROPIONE:Rheumatoid arthritis:0.51), (FLORBETAPIR F-18:Rheumatoid arthritis:0.27), (FLUPENTIXOL:Rheumatoid arthritis:0.34), (FLURBIPROFEN AXETIL:Rheumatoid arthritis:0.28), (FOLIC ACIDIRON:Rheumatoid arthritis:0.36), (FOSFLUCONAZOLE:Rheumatoid arthritis:0.29), (FOSINOPRIL SODIUMHYDROCHLOROTHIAZIDE:Rheumatoid arthritis:0.51), (FOSPHENYTOINFOSPHENYTOIN SODIUM:Rheumatoid arthritis:0.42), (FRANGULA PURSHIANA BARK:Rheumatoid arthritis:0.49), (GABEXATE MESYLATE:Rheumatoid arthritis:0.1), (GADOBUTROL:Rheumatoid arthritis:0.14), (GADOPENTETATE DIMEGLUMINE:Rheumatoid arthritis:0.21), (GADOTERATE MEGLUMINE:Rheumatoid arthritis:0.02), (GENTIAN VIOLET:Rheumatoid arthritis:1.17), (GLATIRAMER:Rheumatoid arthritis:0.76), (GLIMEPIRIDEROSIGLITAZONE MALEATE:Rheumatoid arthritis:0.81), (GLUCONATE:Rheumatoid arthritis:0.55), (GLUCOSAMINE HYDROCHLORIDE:Rheumatoid arthritis:0.82), (GLYCERINMINERAL OILPARAFFIN:Rheumatoid arthritis:0.07), (GRAPEFRUIT SEED EXTRACT:Rheumatoid arthritis:0.23), (GREEN TEA LEAF:Rheumatoid arthritis:1.32), (GUAIAZULENE:Rheumatoid arthritis:0.29), (GUANABENZ ACETATE:Rheumatoid arthritis:0.99), (HERBALSKARAYA GUM:Rheumatoid arthritis:0.76), (HIGH DENSITY POLYETHYLENE:Rheumatoid arthritis:0.36), (HOMATROPINE HYDROBROMIDE:Rheumatoid arthritis:0.56), (HUMAN COLOSTRUM:Rheumatoid arthritis:0.77), (HYALURONIC ACIDLIDOCAINE HYDROCHLORIDE:Rheumatoid arthritis:0.21), (HYDROCORTISONE PROBUTATE:Rheumatoid arthritis:0.95), (HYDROXOCOBALAMIN ACETATE:Rheumatoid arthritis:0.77), (IDARUCIZUMAB:Rheumatoid arthritis:1.38), (IDELALISIB:Rheumatoid arthritis:0.34), (IFENPRODIL:Rheumatoid arthritis:1.2), (IMIPRAMINE PAMOATE:Rheumatoid arthritis:0.32), (INFLUENZA A VIRUS A/BRISBANE/10/2010 (H1N1) ANTIGEN (MDCK CELL DERIVED, PROPIOLACTONE INACTIVATED)\INFLUENZA A VIRUS A/VICTORIA/361/2011 IVR-165 (H3N2) ANTIGEN (MDCK CELL DERIVED, PROPIOLACTONE INACTIVATED)\INFLUENZA B VIRUS B/WISCONSIN/1/2010) ANTIGEN (MDCK CELL DERIVED, PROPIOLACTONE INACTIVATED):Rheumatoid arthritis:0.81), (INFLUENZA A VIRUS A/CALIFORNIA/7/2009 (H1N1) RECOMBINANT HEMAGGLUTININ ANTIGEN\INFLUENZA A VIRUS A/VICTORIA/361/2011 (H3N2) RECOMBINANT HEMAGGLUTININ ANTIGEN\INFLUENZA B VIRUS B/WISCONSIN/1/2010 RECOMBINANT HEMAGGLUTININ ANTIGEN:Rheumatoid arthritis:0.38), (INSULIN ASPARTINSULIN DEGLUDEC:Rheumatoid arthritis:0.67), (INTERFERON ALFA-2A:Rheumatoid arthritis:1.55), (INTERFERON ALFA-2A OR INTERFERON ALFA-2B:Rheumatoid arthritis:0.36), (INTERFERON BETA:Rheumatoid arthritis:0.88), (INTERFERON GAMMA-1B:Rheumatoid arthritis:1.31), (INULIN:Rheumatoid arthritis:0.25), (IODINE\POTASSIUM IODIDE:Rheumatoid arthritis:0.92), (IPRAGLIFLOZIN:Rheumatoid arthritis:0.44), (IRBESARTANTRICHLORMETHIAZIDE:Rheumatoid arthritis:0.13), (IRINOTECAN HYDROCHLORIDE:Rheumatoid arthritis:1.09), (ISAVUCONAZONIUM SULFATE:Rheumatoid arthritis:0), (ISOPROPYL MYRISTATEPARAFFIN:Rheumatoid arthritis:0), (JWH-018:Rheumatoid arthritis:0.36), (KERATIN:Rheumatoid arthritis:0.17), (KETAMINE HYDROCHLORIDE:Rheumatoid arthritis:0.72), (LANDIOLOL HYDROCHLORIDE:Rheumatoid arthritis:0.29), (LANOLINMINERAL OIL:Rheumatoid arthritis:0.23), (LANOLINMINERAL OILPETROLATUM:Rheumatoid arthritis:0.65), (LANOLINPETROLATUM:Rheumatoid arthritis:0.18), (LANREOTIDE:Rheumatoid arthritis:1.29), (LANREOTIDE ACETATE:Rheumatoid arthritis:0.58), (LAVENDER OIL:Rheumatoid arthritis:0.79), (LECITHIN\SODIUM BICARBONATE\SODIUM PHOSPHATE, MONOBASIC:Rheumatoid arthritis:0.24), (LESINURAD:Rheumatoid arthritis:0.8), (LETROZOLERIBOCICLIB:Rheumatoid arthritis:0.5), (LEVOBUNOLOL HYDROCHLORIDE:Rheumatoid arthritis:0.68), (LEVOCARNITINE HYDROCHLORIDE:Rheumatoid arthritis:0.98), (LEVOMEFOLIC ACID:Rheumatoid arthritis:0.76), (LEVORPHANOL:Rheumatoid arthritis:0.18), (LEVORPHANOL TARTRATE:Rheumatoid arthritis:0), (LIGHT MINERAL OILMINERAL OIL:Rheumatoid arthritis:0.1), (LITHIUM OROTATE:Rheumatoid arthritis:0.08), (LIXISENATIDE:Rheumatoid arthritis:0.03), (LOMUSTINE:Rheumatoid arthritis:0.22), (LURASIDONE:Rheumatoid arthritis:0.23), (LUTETIUM LU-177:Rheumatoid arthritis:1.36), (LYNESTRENOL:Rheumatoid arthritis:0.03), (LYSINE HYDROCHLORIDE:Rheumatoid arthritis:0.57), (MAGNESIUM ACETATE:Rheumatoid arthritis:0.38), (MAGNESIUM GLUCEPTATE:Rheumatoid arthritis:0.16), (MAGNESIUM LACTATE:Rheumatoid arthritis:0.07), (MALIC ACID:Rheumatoid arthritis:0.04), (MATRICARIA RECUTITA:Rheumatoid arthritis:0.45), (MECLOZINE DIHYDROCHLORIDE:Rheumatoid arthritis:0.91), (MEFLOQUINE:Rheumatoid arthritis:0.05), (MENINGOCOCCAL POLYSACCHARIDE VACCINE ANEISSERIA MENINGITIDIS GROUP A CAPSULAR POLYSACCHARIDE ANTIGEN:Rheumatoid arthritis:0.13), (MEPENZOLATE BROMIDE:Rheumatoid arthritis:0.75), (METAPROTERENOL SULFATE:Rheumatoid arthritis:0.08), (METHYCLOTHIAZIDE:Rheumatoid arthritis:0.37), (METHYSERGIDE MALEATE:Rheumatoid arthritis:0.21), (METOPIMAZINE:Rheumatoid arthritis:0.1), (MIGLUSTAT:Rheumatoid arthritis:0.6), (MILRINONE LACTATE:Rheumatoid arthritis:0.07), (MINERALSPROBIOTICS NOSVITAMINS:Rheumatoid arthritis:0.18), (MORNIFLUMATE:Rheumatoid arthritis:0.78), (MOSAPRIDE CITRATE DIHYDRATE:Rheumatoid arthritis:0.76), (NABIXIMOLS:Rheumatoid arthritis:0.93), (NADH:Rheumatoid arthritis:0.46), (NAFRONYL:Rheumatoid arthritis:0.3), (NALBUPHINE HYDROCHLORIDE:Rheumatoid arthritis:1.58), (NALDEMEDINE:Rheumatoid arthritis:0.04), (NALFURAFINE HYDROCHLORIDE:Rheumatoid arthritis:0.78), (NALOXONE HYDROCHLORIDEPENTAZOCINE HYDROCHLORIDE:Rheumatoid arthritis:0.98), (NATTOKINASE:Rheumatoid arthritis:0.26), (NEISSERIA MENINGITIDIS GROUP A CAPSULAR OLIGOSACCHARIDE DIPHTHERIA CRM197 PROTEIN CONJUGATE ANTIGEN\NEISSERIA MENINGITIDIS GROUP C CAPSULAR OLIGOSACCHARIDE DIPHTHERIA CRM197 PROTEIN CONJUGATE ANTIGEN\NEISSERIA MENINGITIDIS GROUP W-135 CAPSULAR OLIGOSACCHARIDE DIPHTHERIA CRM197 PROTEIN CONJUGATE ANTI:Rheumatoid arthritis:0.23), (NEISSERIA MENINGITIDIS GROUP A CAPSULAR POLYSACCHARIDE DIPHTHERIA TOXOID CONJUGATE ANTIGEN\NEISSERIA MENINGITIDIS GROUP C CAPSULAR POLYSACCHARIDE DIPHTHERIA TOXOID CONJUGATE ANTIGEN\NEISSERIA MENINGITIDIS GROUP W-135 CAPSULAR POLYSACCHARIDE DIPHTHERIA TOXOID CONJUGATE ANTIGEN\NEISSERIA MENINGITIDIS:Rheumatoid arthritis:0.23), (NEOSTIGMINE:Rheumatoid arthritis:0.28), (NEOSTIGMINE METHYLSULFATE:Rheumatoid arthritis:0.07), (NIACINAMIDEPYRIDOXINE\RIBOFLAVINTHIAMINE:Rheumatoid arthritis:0.05), (NIRAPARIB:Rheumatoid arthritis:0.72), (NITROGEN:Rheumatoid arthritis:0.35), (NUSINERSEN:Rheumatoid arthritis:0.43), (OCTOTIAMINE:Rheumatoid arthritis:0.1), (OCTREOTIDE:Rheumatoid arthritis:0.37), (OLANZAPINE PAMOATE:Rheumatoid arthritis:0.3), (OLAPARIB:Rheumatoid arthritis:0.28), (OLODATEROLTIOTROPIUM BROMIDE:Rheumatoid arthritis:0.03), (OMEGA-3-CARBOXYLIC ACIDS:Rheumatoid arthritis:0.72), (OXANDROLONE:Rheumatoid arthritis:0.2), (OXETORONE:Rheumatoid arthritis:0.11), (OXICONAZOLE NITRATE:Rheumatoid arthritis:0.45), (OXYQUINOLINE SULFATE\SODIUM LAURYL SULFATE:Rheumatoid arthritis:0.14), (PALIPERIDONE PALMITATE:Rheumatoid arthritis:1.24), (PALONOSETRON:Rheumatoid arthritis:0.38), (PANCRELIPASETYROSINE:Rheumatoid arthritis:0.33), (PARSLEY:Rheumatoid arthritis:0.84), (PASCOPYRUM SMITHII POLLEN:Rheumatoid arthritis:0.14), (PASSIFLORA INCARNATA FLOWER:Rheumatoid arthritis:0.55), (PEGINTERFERON ALFA-2A OR PEGINTERFERON ALFA-2B:Rheumatoid arthritis:0.47), (PEGINTERFERON BETA-1A:Rheumatoid arthritis:2.63), (PENBUTOLOL SULFATE:Rheumatoid arthritis:0.16), (PENICILLIN V BENZATHINE:Rheumatoid arthritis:0.65), (PENTAMIDINE ISETHIONATE:Rheumatoid arthritis:1.44), (PEPPERMINT:Rheumatoid arthritis:0.72), (PERAMPANEL:Rheumatoid arthritis:0.11), (PERFLUTREN:Rheumatoid arthritis:0.22), (PEROSPIRONE HYDROCHLORIDE:Rheumatoid arthritis:0.38), (PHENDIMETRAZINE:Rheumatoid arthritis:0.21), (PHENOTHIAZINE:Rheumatoid arthritis:0.38), (PHENYLEPHRINE HYDROCHLORIDEPROMETHAZINE HYDROCHLORIDE:Rheumatoid arthritis:1.47), (PHENYLEPHRINE HYDROCHLORIDEWITCH HAZEL:Rheumatoid arthritis:1.98), (PHOSPHATIDYL SERINE:Rheumatoid arthritis:0.1), (PIPOBROMAN:Rheumatoid arthritis:0.05), (PIRBUTEROL ACETATE:Rheumatoid arthritis:1.91), (PIRENZEPINE:Rheumatoid arthritis:0.31), (PIRMENOL HYDROCHLORIDE:Rheumatoid arthritis:0.12), (PITOFENONE:Rheumatoid arthritis:1.1), (PLERIXAFOR:Rheumatoid arthritis:0.09), (POLYMYXIN B SULFATETRIMETHOPRIM SULFATE:Rheumatoid arthritis:0.01), (POLYSTYRENE:Rheumatoid arthritis:0.96), (POTASSIUM CHLORIDE\SODIUM CHLORIDE:Rheumatoid arthritis:0.13), (POTASSIUM CHLORIDE\SODIUM CHLORIDE\SODIUM LACTATE:Rheumatoid arthritis:1), (POTASSIUM PHOSPHATE:Rheumatoid arthritis:0.22), (POTASSIUM PHOSPHATE, MONOBASIC\SODIUM PHOSPHATE, DIBASIC, ANHYDROUS\SODIUM PHOSPHATE, MONOBASIC, MONOHYDRATE:Rheumatoid arthritis:0.12), (POVIDONE:Rheumatoid arthritis:1.43), (PROMAZINE HYDROCHLORIDE:Rheumatoid arthritis:0.37), (PROPANTHELINE:Rheumatoid arthritis:0.06), (PROPANTHELINE BROMIDE:Rheumatoid arthritis:1.02), (PROPOXYPHENE NAPSYLATE:Rheumatoid arthritis:0.36), (PSORALEN:Rheumatoid arthritis:1.2), (PYGEUM:Rheumatoid arthritis:0.89), (QUINAGOLIDE HYDROCHLORIDE:Rheumatoid arthritis:0.29), (QUINIDINE GLUCONATE:Rheumatoid arthritis:0.65), (RADIATION THERAPY:Rheumatoid arthritis:1.76), (RANITIDINE BISMUTH CITRATE:Rheumatoid arthritis:0.07), (RASBURICASE:Rheumatoid arthritis:0.3), (RASPBERRY KETONE:Rheumatoid arthnitis:0.52), (REBOXETINE MESYLATE:Rheumatoid arthritis:0.49), (RED CLOVER:Rheumatoid arthritis:0.82), (RED YEAST:Rheumatoid arthritis:0.39), (REGORAFENIB:Rheumatoid arthritis:1.21), (RESERPINE:Rheumatoid arthritis:0.47), (RHUBARB:Rheumatoid arthritis:1.15), (RIBOCICLIB:Rheumatoid arthritis:0.03), (RICOLINOSTAT:Rheumatoid arthritis:0.22), (RILONACEPT:Rheumatoid arthritis:0.3), (RILPIVIRINE:Rheumatoid arthritis:0.22), (ROMOSOZUMAB:Rheumatoid arthritis:0.04), (RUCAPARIB CAMSYLATE:Rheumatoid arthritis:0.15), (SAFINAMIDE:Rheumatoid arthritis:0.24), (SAFINAMIDE MESYLATE:Rheumatoid arthritis:0.19), (SALICYLAMIDE:Rheumatoid arthritis:0), (SAPROPTERIN DIHYDROCHLORIDE:Rheumatoid arthritis:0.58), (SELENIUM SULFIDE:Rheumatoid arthritis:0.85), (SERMORELIN ACETATE:Rheumatoid arthritis:0.12), (SERTACONAZOLE NITRATE:Rheumatoid arthritis:0.14), (SERTINDOLE:Rheumatoid arthritis:0.26), (SEVELAMER:Rheumatoid arthritis:0.72), (SEVOFLURANE:Rheumatoid arthritis:0.42), (SILVER NITRATE:Rheumatoid arthritis:0.14), (SIPULEUCEL-T:Rheumatoid arthritis:0.39), (SODIUM BICARBONATE\SODIUM PHOSPHATE, MONOBASIC:Rheumatoid arthritis:0.18), (SODIUM CITRATE:Rheumatoid arthritis:0.53), (SODIUM FLUORIDEVITAMINS:Rheumatoid arthritis:0.59), (SODIUM HYDROXIDE:Rheumatoid arthritis:0.21), (SODIUM PHOSPHATE, DIBASIC:Rheumatoid arthritis:0.32), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Rheumatoid arthritis:0.62), (SODIUM PHOSPHATE, DIBASIC, ANHYDROUS\SODIUM PHOSPHATE, MONOBASIC, MONOHYDRATE:Rheumatoid arthritis:0.04), (SODIUM PHOSPHATE, MONOBASIC, ANHYDROUS:Rheumatoid arthritis:0.24), (STREPTOMYCIN SULFATE:Rheumatoid arthritis:0.19), (STREPTOMYCIN\STREPTOMYCIN SULFATE:Rheumatoid arthritis:0.54), (STRONTIUM:Rheumatoid arthritis:0.76), (STRONTIUM CHLORIDE:Rheumatoid arthritis:0.6), (SUFENTANIL CITRATE:Rheumatoid arthritis:0.49), (SULBUTIAMINE:Rheumatoid arthritis:0.46), (TABALUMAB:Rheumatoid arthritis:0.22), (TALIGLUCERASE ALFA:Rheumatoid arthritis:1.07), (TANDOSPIRONE:Rheumatoid arthritis:0.41), (TAR:Rheumatoid arthritis:0.12), (TBO-FILGRASTIM:Rheumatoid arthritis:0.21), (TEDUGLUTIDE:Rheumatoid arthritis:0.63), (TEDUGLUTIDE\WATER:Rheumatoid arthritis:0.86), (TEGAFURURACIL:Rheumatoid arthritis:0.02), (TEGASEROD:Rheumatoid arthritis:0.86), (TEGASEROD MALEATE:Rheumatoid arthritis:2.19), (TELOTRISTAT ETHYL:Rheumatoid arthritis:0.19), (TEMOCAPRIL HYDROCHLORIDE:Rheumatoid arthritis:1.39), (TEMSIROLIMUS:Rheumatoid arthritis:0.16), (TETANUS ANTITOXIN:Rheumatoid arthritis:0.29), (THIABENDAZOLE:Rheumatoid arthritis:0.22), (THIOPENTAL SODIUM:Rheumatoid arthritis:0.27), (THIOTHIXENE:Rheumatoid arthritis:0.59), (THIOTHIXENE HYDROCHLORIDE:Rheumatoid arthritis:0.12), (TIARAMIDE:Rheumatoid arthritis:0.82), (TIMOLOLTRAVOPROST:Rheumatoid arthritis:2.01), (TIPEPIDINE HIBENZATE:Rheumatoid arthritis:0.88), (TIQUIZIUM BROMIDE:Rheumatoid arthritis:1.18), (TOLCAPONE:Rheumatoid arthritis:0.3), (TOLPERISONE HYDROCHLORIDE:Rheumatoid arthritis:0.18), (TOREMIFENE CITRATE:Rheumatoid arthritis:0.53), (TRAMETINIB DIMETHYL SULFOXIDE:Rheumatoid arthritis:0.13), (TRANILAST:Rheumatoid arthritis:0.31), (TREPROSTINIL SODIUM:Rheumatoid arthritis:0.26), (TRICHLOROACETIC ACID:Rheumatoid arthritis:0.6), (TRICLOSAN:Rheumatoid arthritis:0.68), (TRIMETHOPRIM SULFATE:Rheumatoid arthritis:0.69), (TRIPTORELIN:Rheumatoid arthritis:0.82), (TRIPTORELIN PAMOATE:Rheumatoid arthritis:1.77), (TROGLITAZONE:Rheumatoid arthritis:1), (TROLAMINE:Rheumatoid arthritis:0.17), (TROXERUTIN:Rheumatoid arthritis:0.45), (UNDECYLENIC ACID:Rheumatoid arthritis:0.23), (UROKINASE:Rheumatoid arthritis:0.55), (VANDETANIB:Rheumatoid arthritis:0.43), (VELAGLUCERASE ALFA:Rheumatoid arthritis:0.88), (VINBLASTINE SULFATE:Rheumatoid arthritis:0.07), (VITAMIN A PALMITATE:Rheumatoid arthritis:0.75), (XYLITOL:Rheumatoid arthritis:0.39), (ZANAMIVIR:Rheumatoid arthritis:0.12), (ZINC PICOLINATE:Rheumatoid arthritis:0.22), (ZIV-AFLIBERCEPT:Rheumatoid arthritis:0.4), (ZUCLOPENTHIXOL:Rheumatoid arthritis:0.36), (.ALPHA.1-PROTEINASE INHIBITOR HUMAN:Rhinitis:0.97), (ACARBOSE:Rhinitis:0.16), (ACETAMINOPHENOXYCODONEOXYCODONE HYDROCHLORIDE:Rhinitis:0.38), (AGALSIDASE BETA:Rhinitis:0), (ALBIGLUTIDE:Rhinitis:0.01), (ALCOHOL:Rhinitis:0.22), (ALEMTUZUM AB:Rhinitis:0.09), (AMANTADINE:Rhinitis:0.19), (AMIODARONE:Rhinitis:1.34), (AMLODIP INEVALSARTAN:Rhinitis:0.04), (ATROPINE SULFATEDIPHENOXYLATE HYDROCHLORIDE:Rhinitis:0.19), (BENAZEPRIL HYDROCHLORIDE:Rhinitis:0.6), (BENAZEPRILBENAZEPRIL HYDROCHLORIDE:Rhinitis:0.45), (BICALUTAMIDE:Rhinitis:0.28), (BROTIZOLAM:Rhiniti s:0.2), (BUPRENORPHINE HYDROCHLORIDE:Rhinitis:0.14), (BUPRENORPHINE HYDROCHLORIDENALOXONE HYDROCHLORIDE:Rhinitis:0.29), (CALCITRIOL:Rhinitis:0.72), (CALCIUM ACETATE:Rhinitis:0.59), (CALCIUM\MAGNESIUM:Rhinitis:0.11), (CANDESARTAN CILEXETILHYDROCHLOROTHIAZIDE:Rhinitis:1.07), (CARVEDILOL PHOSPHATE:Rhinitis:1.01), (CEFEPIME HYDROCHLORIDE:Rhinitis:0.08), (CHLORDIAZEPOXIDE HYDROCHLORIDECLIDINIUM BROMIDE:Rhinitis:0.02), (CHLORPHENIRAMINEHYDROCODONE:Rhinitis:0.1), (CILAST ATIN SODIUMIMIPENEM:Rhinitis:0.37), (CIMETIDINE:Rhinitis:0.74), (CLOZAPINE:Rhinitis:0.93), (COD LIVER OIL:Rhinitis:0.07), (COLESEVELAM HYDROCHLORIDE:Rhinitis:0.28), (CORTICOTROPIN:Rhinitis:0.09), (CORTISONEHYDRO CORTISONE:Rhinitis:1.43), (CRIZOTINIB:Rhinitis:0.15), (CYPROHEPTADINE:Rhinitis:0.14), (DACLIZUMAB:Rhinitis:0.8), (DALTEPARIN:Rhinitis:0.13), (DALTEPARIN SODIUM:Rhinitis:0.3), (DANAZOL:Rhinitis:0.42), (DASATINIB:Rhinitis:1.71), (DEXAMETH ASONE ACETATE:Rhinitis:0.43), (DOCUSATE:Rhinitis:0.85), (DOCUSATE CALCIUM:Rhinitis:0.4), (DRONABINOL:Rhinitis:0.67), (ECONAZOLE:Rhinitis:0.21), (EDOX ABAN TOSYLATE:Rhinitis:0.54), (EPIRUBICIN:Rhinitis:0.04), (EPLERENONE:Rhinitis:0.33), (ESO MEPRAZOLE MAGNESIUMNAPROXEN:Rhinitis:0.01), (ESZOPICLONE:Rhinitis:1.42), (ETORICOXIB:Rh initis:0.68), (EVOLOCUMAB:Rhinitis:0.88), (FENOTEROL HYDROBROMIDEIPRATROPIUM BROMIDE:Rhinitis:0.18), (FENTANYL CITRATE:Rhinitis:0.1), (FERROUS FUMARATE:Rhinitis:0.07), (FLECAINIDE:Rhinitis:0.99), (FLUVOXAMINE MALEATE:Rhinitis:0.04), (FORMOTEROL FUMARATE\MOMETASONE FUROATE:Rhinitis:0.97), (GLICLAZIDE:Rhinitis:0.63), (GLYCOPYRROLATE:Rhinitis:0.2), (GLYCOPYRRONIUM:Rhinitis:0.5), (HERBALSTURMERIC:Rhinitis:0.07), (HYDROXYZINE PAMOATE:Rhinitis:1.35), (HYOSCYAMINE:Rhinitis:0.45), (ILOPROST:Rhinitis:1.13), (INDA PAMIDEPERINDOPRIL:Rhinitis:0.75), (INSULIN DETEMIR:Rhinitis:0.3), (IRINOTECAN HYDROCHLORIDE:Rhinitis:0.01), (KETOROLACKETOROLAC TROMETHAMINE:Rhinitis:0.08), (LEDIPASVIRSOFOSBUVIR:Rhinitis:0.67), (LERCANIDI PINE HYDROCHLORIDE:Rhinitis:0.57), (LITHIUM CARBONATE:Rhinitis:0.97), (LUMACAFTOR:Rhinitis:0.05), (MEROPENEM:Rhinitis:0.38), (METHENAMINE HIPPURATE:Rhinitis:0.32), (METOLAZONE:Rhinitis:0.25), (MOXIFLOXACIN:Rhinitis:0.31), (NADOLOL:Rhinitis:0.28), (NALOXONE HYDROCHLORIDEOXYCODONE HYDROCHLORIDE:Rhinitis:0.68), (NORETHINDRONE:Rhinitis:0.48), (PANTOPRAZOLE MAGNESIUM:Rhinitis:1.82), (PAROXETINEPAROXETINE HYDROCHLORIDE:Rhinitis:0.89), (PAZOPANIB HYDROCHLORIDE:Rhinitis:0.66), (PEGINTERFERON BETA-1A:Rhinitis:0.46), (PEMETREXED DISODIUM:Rhinitis:0.46), (PENICILLIN G:Rhinitis:0.11), (PERINDOPRIL:Rhinitis:0.57), (PHYTONADIONE:Rhinitis:0.44), (PRIMIDO NE:Rhinitis:1.2), (RABEPRAZOLE:Rhinitis:0.72), (RIOCIGUAT:Rhinitis:0.72), (SACUBITRIL VALSARTAN:Rhinitis:0.96), (SALMETEROL XINAFOATE:Rhinitis:0.28), (SODIUM FLUORIDE:Rhinitis:0.15), (TETRACYCLINE:Rhinitis:0.67), (THEOPHYLLINE:Rhinitis:0.06), (TINZAPARIN SODIUM:Rhinitis:0.24), (TIZANIDINE:Rhinitis:0.71), (TOCOPHEROL:Rhinitis:0.07), (TRIAM TERENE:Rhinitis:0.55), (UMECLIDINIUM BROMIDE:Rhinitis:0.16), (VALGANCICLOVIR HYDROCHLORIDE:Rhinitis:0.22), (VANCOMYCIN HYDROCHLORIDE:Rhinitis:0.35), (VILAZODONE HYDROCHLORIDE:Rhinitis:0.39), (ZILEUTON:Rhinitis:0.22), (ZONISAMIDE:Rhinitis:0.14), (ABACAVIR:Rhinitis allergic:0.03), (ABACAVIR SULFATEDOLUTEGRAVIR SODIUMLAMIVUDINE:Rhinitis allergic:0.35), (ABOBOTULINUMTOXINA:Rhinitis allergic:0.14), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Rhinitis allergic:0.31), (ACETAMINOPHENOXYCODONEOXYCODONE HYDROCHLORIDE:Rhinitis allergic:1.1), (ACETAMINOPHENPROPOXYPHENE NAPSYLATE:Rhinitis allergic:0.55), (ACETAMINOPHENPSEUDOEPHEDRINE HYDROCHLORIDE:Rhinitis allergic:0.08), (ADENINE:Rhinitis allergic:0.69), (AGALSIDASE BETA:Rhinitis allergic:0.47), (ALBIGLUTIDE:Rhinitis allergic:0.7), (ALBUTEROLGUAIFENESIN:Rhinitis allergic:0.26), (ALISKIREN:Rhinitis allergic:0.39), (AMANTADINE:Rhinitis allergic:0.19), (AMILORIDE HYDROCHLORIDEHYDROCHLOROTHIAZIDE:Rhinitis allergic:0.36), (AMINO ACIDSCARBOHYDRATESELECTROLYTES NOSVITAMINS:Rhinitis allergic:0.3), (AMISULPRIDE:Rhinitis allergic:0.68), (AMLODIPINE BESYLATEHYDROCHLOROTHIAZIDEVALSARTAN:Rhinitis allergic:0), (AMLODIPINE BESYLATEIRBESARTAN:Rhinitis allergic:0.8), (AMLODIPINEVALSARTAN:Rhinitis allergic:0.32), (AMOXICILLINCLARITHROMYCINESOMEPRAZOLE:Rhinitis allergic:0.1), (AMPHOTERICIN B:Rhinitis allergic:0.32), (AMPICILLIN:Rhinitis allergic:0.56), (ASPIRINMAGNESIUM OXIDE:Rhinitis allergic:0.18), (BALSALAZIDE DISODIUM:Rhinitis allergic:0.02), (BENAZEPRILBENAZEPRIL HYDROCHLORIDE:Rhinitis allergic:1.01), (BENZBROMARONE:Rhinitis allergic:0.51), (BENZTROPINE MESYLATE:Rhinitis allergic:0.02), (BEPRIDIL:Rhinitis allergic:1.66), (BERAPROST:Rhinitis allergic:0.38), (BETAMETHASONE DIPROPIONATECALCIPOTRIENECALCIPOTRIENE HYDRATE:Rhinitis allergic:0.72), (BETAMETHASONE DIPROPIONATECLOTRIMAZOLE:Rhinitis allergic:0.03), (BETAMETHASONE SODIUM PHOSPHATENEOMYCIN SULFATE:Rhinitis allergic:0.3), (BETAMETHASONECALCIPOTRIENE:Rhinitis allergic:0.74), (BETHANECHOL CHLORIDE:Rhinitis allergic:0.13), (BICARBONATE IONPOLYETHYLENE GLYCOLSPOTASSIUM CATION\SODIUM CHLORIDE\SODIUM SULFATE ANHYDROUS:Rhinitis allergic:0.17), (BIPERIDEN HYDROCHLORIDE:Rhinitis allergic:0.03), (BUTALBITAL:Rhinitis allergic:0.11), (CABAZITAXEL:Rhinitis allergic:0.07), (CALCITONIN:Rhinitis allergic:0.32), (CALCIUM CARBONATEERGOCALCIFEROLRETINOL:Rhinitis allergic:0.48), (CALCIUM CHLORIDE ANHYDROUSPOTASSIUM CHLORIDE\SODIUM CHLORIDE\SODIUM LACTATE:Rhinitis allergic:0.18), (CALCIUM\MAGNESIUM:Rhinitis allergic:0.56), (CALCIUM\MAGNESIUM\ZINC:Rhinitis allergic:0.15), (CANAGLIFLOZINMETFORMIN HYDROCHLORIDE:Rhinitis allergic:0.92), (CARVEDILOL PHOSPHATE:Rhinitis allergic:1.44), (CEFAZOLIN:Rhinitis allergic:0.19), (CEFTERAM PIVOXIL:Rhinitis allergic:0.93), (CEPHARANTHINE:Rhinitis allergic:0.75), (CERITINIB:Rhinitis allergic:0.14), (CHLORAMBUCIL:Rhinitis allergic:0.31), (CILASTATIN SODIUMIMIPENEM:Rhinitis allergic:0.89), (CLOPERASTINE:Rhinitis allergic:0.39), (CRIZOTINIB:Rhinitis allergic:1.23), (DABIGATRAN:Rhinitis allergic:0.63), (DABRAFENIB:Rhinitis allergic:0.05), (DACLATASVIR DIHYDROCHLORIDE:Rhinitis allergic:0.02), (DACLIZUMAB:Rhinitis allergic:0.92), (DALTEPARIN:Rhinitis allergic:0.83), (DANAZOL:Rhinitis allergic:0.02), (DAPAGLIFLOZIN PROPANEDIOL:Rhinitis allergic:0.74), (DASABUVIROMBITASVIRPARITAPREVIRRITONAVIR:Rhinitis allergic:0.19), (DASATINIB:Rhinitis allergic:2.78), (DEFLAZACORT:Rhinitis allergic:1.32), (DEGARELIX:Rhinitis allergic:0.54), (DEPRODONE PROPIONATE:Rhinitis allergic:0.17), (DIAZOXIDE:Rhinitis allergic:0.07), (DIFLUCORTOLONE VALERATELIDOCAINE:Rhinitis allergic:0.31), (DIGITOXIN:Rhinitis allergic:0.08), (DIMEMORFAN PHOSPHATE:Rhinitis allergic:0.52), (DIMENHYDRINATE:Rhinitis allergic:1.25), (DISOPYRAMIDE:Rhinitis allergic:0.02), (DOFETILIDE:Rhinitis allergic:1.02), (DOLUTEGRAVIR:Rhinitis allergic:0.15), (DONEPEZIL:Rhinitis allergic:0.12), (DORZOLAMIDE HYDROCHLORIDE:Rhinitis allergic:0.18), (DOXORUBICIN HYDROCHLORIDE:Rhinitis allergic:0.09), (DOXYLAMINE SUCCINATE:Rhinitis allergic:0.07), (DULAGLUTIDE:Rhinitis allergic:0.23), (ECABET SODIUM:Rhinitis allergic:0.2), (ECONAZOLE:Rhinitis allergic:0.68), (EDARAVONE:Rhinitis allergic:0.14), (EDOXABAN TOSYLATE:Rhinitis allergic:1.79), (ELBASVIR:Rhinitis allergic:0.1), (ELOTUZUMAB:Rhinitis allergic:0.74), (EMPAGLIFLOZINLINAGLIPTIN:Rhinitis allergic:0.48), (ENALAPRIL MALEATEHYDROCHLOROTHIAZIDE:Rhinitis allergic:0.05), (ETHACRYNIC ACID:Rhinitis allergic:0.02), (ETHINYL ESTRADIOLGESTODENE:Rhinitis allergic:0.06), (ETHYL ICOSAPENTATEICOSAPENT:Rhinitis allergic:0.83), (ETOPOSIDE:Rhinitis allergic:0.04), (ETRETINATE:Rhinitis allergic:2.04), (FAROPENEM SODIUM:Rhinitis allergic:0.82), (FENOTEROL HYDROBROMIDE:Rhinitis allergic:1.08), (FERRIC HYDROXIDE:Rhinitis allergic:0.28), (FERROUS SULFATEGLYCINE:Rhinitis allergic:0.2), (FLAVIN ADENINE DINUCLEOTIDE:Rhinitis allergic:1.9), (FORMOTEROL FUMARATE DIHYDRATE:Rhinitis allergic:0.21), (FULVESTRANT:Rhinitis allergic:0.26), (FURSULTIAMINE:Rhinitis allergic:0.63), (GIMERACILOTERACILTEGAFUR:Rhinitis allergic:1.14), (GLUCONATE:Rhinitis allergic:0.03), (GLYCOPYRROLATEINDACATEROL MALEATE:Rhinitis allergic:0.5), (GRAZOPREVIR:Rhinitis allergic:0.07), (GUANFACINE HYDROCHLORIDE:Rhinitis allergic:0.55), (HEPARIN CALCIUM:Rhinitis allergic:0.01), (HERBALSTURMERIC:Rhinitis allergic:0.54), (HYDROCODONE BITARTRATEIBUPROFEN:Rhinitis allergic:0.4), (HYDROCORTISONE PROBUTATE:Rhinitis allergic:0.6), (IGURATIMOD:Rhinitis allergic:0.52), (IMIGLUCERASE:Rhinitis allergic:0.06), (IMIPRAMINE HYDROCHLORIDE:Rhinitis allergic:1.03), (INFLIXIMAB-DYYB:Rhinitis allergic:1.07), (INTERFERON:Rhinitis allergic:0.6), (INTERFERON ALFA-2A:Rhinitis allergic:0.07), (INTERFERON ALFA-2B:Rhinitis allergic:0), (IOPROMIDE:Rhinitis allergic:0.73), (IRINOTECAN HYDROCHLORIDE:Rhinitis allergic:0.45), (ISONIAZIDRIFAMPIN:Rhinitis allergic:0.18), (ISOPROPYL ALCOHOLLEUPROLIDE ACETATENORETHINDRONE ACETATE:Rhinitis allergic:0.32), (IXEKIZUMAB:Rhinitis allergic:0.32), (JUNIPERUS VIRGINIANA POLLEN:Rhinitis allergic:0.38), (LABETALOL HYDROCHLORIDE:Rhinitis allergic:0.79), (LACTOBACILLUS RHAMNOSUS:Rhinitis allergic:0.66), (LANDIOLOL HYDROCHLORIDE:Rhinitis allergic:0), (LEUCOVORIN CALCIUM:Rhinitis allergic:0.95), (LEVOCARNITINE HYDROCHLORIDE:Rhinitis allergic:1.39), (LEVOLEUCOVORIN CALCIUM:Rhinitis allergic:0.45), (LEVOMEFOLATE CALCIUM:Rhinitis allergic:0.12), (LEVOMEFOLATE CALCIUM\METHYLCOBALAMINPYRIDOXAL PHOSPHATE ANHYDROUS:Rhinitis allergic:0.16), (LOXOPROFEN SODIUM DIHYDRATE:Rhinitis allergic:0.29), (LULICONAZOLE:Rhinitis allergic:0.91), (LUMACAFTOR:Rhinitis allergic:0.75), (MEGESTROL:Rhinitis allergic:0.08), (MEPERIDINE:Rhinitis allergic:0.07), (METAMIZOLE:Rhinitis allergic:0.33), (METHENOLONE ENANTHATE:Rhinitis allergic:0.77), (MITIGLINIDE CALCIUM HYDRATEVOGLIBOSE:Rhinitis allergic:0.38), (MORPHINE SULFATE\NALTREXONE HYDROCHLORIDE:Rhinitis allergic:0.05), (NALOXONE HYDROCHLORIDEOXYCODONE HYDROCHLORIDE:Rhinitis allergic:1.42), (NALOXONE HYDROCHLORIDETILIDINE HYDROCHLORIDE:Rhinitis allergic:0.42), (NALTREXONE:Rhinitis allergic:0.21), (NARATRIPTAN HYDROCHLORIDE:Rhinitis allergic:0.06), (NINTEDANIB:Rhinitis allergic:1.81), (OCTREOTIDE:Rhinitis allergic:0.18), (OLMESARTAN:Rhinitis allergic:0.39), (OXAZEPAM:Rhinitis allergic:0.64), (OXYBUTYNINOXYBUTYNIN CHLORIDE:Rhinitis allergic:0.06), (PAMIDRONATE DISODIUM:Rhinitis allergic:0.66), (PANITUMUMAB:Rhinitis allergic:0), (PARICALCITOL:Rhinitis allergic:0.24), (PATIROMER:Rhinitis allergic:0.19), (PAZOPANIB HYDROCHLORIDE:Rhinitis allergic:1.39), (PEMETREXED:Rhinitis allergic:0.67), (PENICILLIN G:Rhinitis allergic:0.17), (PILOCARPINE:Rhinitis allergic:0.36), (PIMOBENDAN:Rhinitis allergic:1.27), (PIOGLITAZONE:Rhinitis allergic:0.03), (PNEUMOCOCCAL VACCINE, POLYVALENT 23:Rhinitis allergic:0.26), (POSACONAZOLE:Rhinitis allergic:0.01), (POTASSIUM CITRATETRISODIUM CITRATE DIHYDRATE:Rhinitis allergic:1.09), (POTASSIUM IODIDE:Rhinitis allergic:0.09), (PRAMIPEXOLE:Rhinitis allergic:1.01), (PRAZOSIN HYDROCHLORIDE:Rhinitis allergic:0.48), (PROTRIPTYLINE HYDROCHLORIDE:Rhinitis allergic:0.13), (PYRIDOSTIGMINE BROMIDE:Rhinitis allergic:0.37), (PYRIDOXAL:Rhinitis allergic:0.45), (QUINACRINE:Rhinitis allergic:0.42), (QUINAPRIL:Rhinitis allergic:0.31), (RADIATION THERAPY:Rhinitis allergic:0.34), (REGORAFENIB:Rhinitis allergic:0.64), (REPAGLINIDE:Rhinitis allergic:0.15), (RILPIVIRINE HYDROCHLORIDE:Rhinitis allergic:0.4), (RIOCIGUAT:Rhinitis allergic:0.94), (ROXITHROMYCIN:Rhinitis allergic:0.03), (SITAFLOXACIN:Rhinitis allergic:0.44), (SULINDAC:Rhinitis allergic:0.15), (TERBUTALINE:Rhinitis allergic:0.27), (TERBUTALINE SULFATE:Rhinitis allergic:2.08), (TERIFLUNOMIDE:Rhinitis allergic:0.48), (TESTOSTERONE UNDECANOATE:Rhinitis allergic:0.78), (TETRACYCLINE:Rhinitis allergic:1.46), (THIAMINE:Rhinitis allergic:0.08), (TINZAPARIN:Rhinitis allergic:0.21), (TOLTERODINE:Rhinitis allergic:0.07), (TRIAMTERENE:Rhinitis allergic:1.18), (VIDARABINE:Rhinitis allergic:0.14), (VINORELBINE TARTRATE:Rhinitis allergic:0.06), (YEAST:Rhinitis allergic:0.15), (ZALTOPROFEN:Rhinitis allergic:0.07), (.ALPHA.1-PROTEINASE INHIBITOR HUMAN:Rhinorrhoea:0.15), (ACARBOSE:Rhinorrhoea:0.14), (ACETAMINOPHENOXYCOD ONEOXYCODONE HYDROCHLORIDE:Rhinorrhoea:0.17), (ARMODAFINIL:Rhinorrhoea:0.99), (ATROPINE SULFATEDIPHENOXYLATE HYDROCHLORIDE:Rhinorrhoea:0.45), (BENAZEPRILBENAZEPRIL HYDROCHLORIDE:Rhinorrhoea:0.16), (BROMAZEPAM:Rhinorrhoea:0.12), (BUDESONIDE FORMOTEROL FUMARATE:Rhinorrhoea:0.3), (BUPRENORPHINE HYDROCHLORIDENALOXONE HYDROCHLORIDE:Rhinorrhoea:0.06), (CALCITRIOL:Rhinorrhoea:1.03), (CANDESARTAN CILEXETILHYDROCHLOROTHIAZIDE:Rhinorrhoea:0.63), (CARISOPRODOL:Rhinorrhoea: 0.31), (CHLORTHALIDONE:Rhinorrhoea:0.27), (CHONDROITIN SULFATE (BOVINE)GLUCOSAMINE HYDROCHLORIDE:Rhinorrhoea:0.05), (CLOZAPINE:Rhinorrhoea:0.01), (COD LIVER OIL:Rhinorrhoea:0), (COLCHICINE:Rhinorrhoea:0.71), (CORTICOTROPIN:Rhinorrhoea:0.01), (DACLIZUMAB:Rhinorrhoea:0.28), (DAPAGLIFLOZIN PROPANEDIOL:Rhinorrhoea:0.06), (DARBEPOETIN ALFA:Rhinorrhoea:0.03), (DEVICE:Rhinorrhoea:0.26), (DOFETILIDE:Rhinorrhoea:0.3), (DOX EPIN HYDROCHLORIDE:Rhinorrhoea:0.14), (DRONABINOL:Rhinorrhoea:0.36), (ELETRIPTAN HYDROBROMIDE:Rhinorrhoea:0.68), (ESTROGENS, CONJUGATEDMEDROXYPROGESTERONE ACETATE:Rhinorrhoea:0.23), (ESZOPICLONE:Rhinorrhoea:0.83), (EVEROLIMUS:Rhinorrho ea:0.67), (FEBUXOSTAT:Rhinorrhoea:0.13), (FESOTERODINE FUMARATE:Rhinorrhoea:0.51), (FLECAINIDE:Rhinorrhoea:0.74), (FLUOCINONIDE:Rhinorr hoea:0.04), (FLUTICASONE FUROATESALMETEROL XINAFOATE:Rhinorrhoea:0.58), (FORMOTEROL FUMARATE:Rhinorrhoea:0.48), (FORMOTEROL FUMARATE\MOMETASONE FUROATE:Rhinorrhoea:0.33), (FULVESTRANT:Rhinorrhoea:0.31), (GLYCOPYRRONIUMIN DACATEROL:Rhinorrhoea:0.32), (HYDROCHLOROTHIAZIDEIRBESARTAN:Rhinorrhoea: 0.3), (ILOPROST:Rhinorrhoea:0.28), (IMIPRAMINE HYDROCHLORIDE:Rhinorrhoea:0.31), (INDAPAMIDEPERINDOPRIL:Rhinorrhoea:0.18), (L ERCANIDIPINE:Rhinorrhoea:0.11), (LINACLOTIDE:Rhinorrhoea:0.24), (LITHIUM CARBONATE:Rhinorrhoea:0.11), (LOMITAPIDE MESYLATE:Rhinorrhoea:0.04), (MEPOLIZUMAB:Rhinorrhoea:0.12), (METHADONE HYDROCHLORIDE:Rhinorrhoea:0.48), (METHENAMINE HIPPURATE:Rhinorrhoea:0.37), (METHYLPREDNISOLONE ACETATE:Rhinorrhoea:0.31), (MIDODRINE:Rhinorrhoea:0.08), (MINOCYCLINEMINOCYC LINE HYDROCHLORIDE:Rhinorrhoea:0.08), (NABUMETONE:Rhinorrhoea:0.2), (NADOLOL:Rhin orrhoea:0.1), (NALOXONE HYDROCHLORIDEOXYCODONE HYDROCHLORIDE:Rhinorrhoea:0.28), (NATEGLINIDE:Rhinorrhoea:0.01), (NILOTINIB:Rhi norrhoea:0.52), (NINTEDANIB:Rhinorrhoea:0.36), (NITROFURANTOINNITROFURANTOIN MONOHYDRATE:Rhinorrhoea:0.03), (OXAZEPAM:Rhinorrhoea:0.05), (PANTOPRAZOLE MAGNESIUM:Rhinorrhoea:1.15), (PAZOPANIB HYDROCHLORIDE:Rhinorrhoea:0.09), (PERINDOPRIL:Rhinorrhoea:0.12), (QUETIAPINE:Rh inorrhoea:0.41), (RABEPRAZOLE SODIUM:Rhinorrhoea:0.66), (RIZATRIPTAN BENZOATE:Rhinorrhoea:0.52), (ROFLUMILAST:Rhinorrhoea:0.73), (SACUBITRILVALSAR TAN:Rhinorrhoea:0.6), (SECUKINUMAB:Rhinorrhoea:0.33), (SULFAMETHOXAZOLE:Rhino rrhoea:0.22), (SUMATRIPTANSUMATRIPTAN SUCCINATE:Rhinorrhoea:0.74), (TERBUTALINE SULFATE:Rhinorrhoea:0.05), (TETRACYCLINE:Rhinorrhoea:0.36), (THALIDOMIDE:Rhinorr hoea:0.01), (TIOTROPIUM:Rhinorrhoea:0.2), (TOCILIZUMAB:Rhinorrhoea:0.18), (ZONISAMI DE:Rhinorrhoea:0.02), (ZOPICLONE:Rhinorrhoea:0.47), (ILOPROST:Right ventricular failure:0.36), (ACARBOSE:Rosacea:0.02), (ACETAMINOPHENOXYCODONE HYDROCHLORIDEOXYCODONE TEREPHTHALATE:Rosacea:0.47), (ACETAZOLAMIDE:Rosacea:0.84), (ALENDRONIC ACID:Rosacea:0.08), (AMANTADINE:Rosacea:0.05), (AMLODIPINE BESYLATEHYDROCHLOROTHIAZIDEOLMESARTAN MEDOXOMIL:Rosacea:0.35), (BACITRACIN:Rosacea:0.13), (BALSALAZIDE DISODIUM:Rosacea:0.12), (BETAMETHASONECALCIPOTRIENE:Rosacea:0.11), (BISOPR OLOL:Rosacea:0.45), (BRIMONIDINE TARTRATETIMOLOL MALEATE:Rosacea:0.3), (BUDESONIDEFORMOTEROL FUMARATE:Rosacea:0.26), (CALCITRIOL:Rosacea:0.26), (CALCIUM ACETATE:Rosacea:0.41), (CALCIUM CARBONATECHOLECALCIFEROL:Rosacea:1.29), (CALCIUM\MAGNESIUM:Rosacea:0.28), (CALCIUM\MAGNESIUM\ZINC:Rosacea:0.44), (CANAGLIFLOZINMETFORMIN HYDROCHLORIDE:Rosacea:0.16), (CANAKINUMAB:Rosacea:0.2), (CAPSAICIN:Rosacea:0.15), (CARFILZOMIB:Rosacea:0.33), (CARVEDILOL PHOSPHATE:Rosacea:1.03), (CHLORDIAZEPOXIDE HYDROCHLORIDECLIDINIUM BROMIDE:Rosacea:0.26), (CIMETIDINE:Rosacea:0.49), (CLOZAPINE:Rosacea:0.2), (CORTIC OTROPIN:Rosacea:0.17), (DASATINIB:Rosacea:0.63), (DIMENHYDRINATE:Rosacea:0.4), (D IPHENHYDRAMINE:Rosacea:1.22), (DOCUSATE:Rosacea:0.51), (DRONABINOL:Rosacea:0.38), (EDOXABAN TOSYLATE:Rosacea:0.12), (EMPAGLIFLOZINLINAGLIPTIN:Rosacea:0.3), (ENOXAPARIN SODIUM:Rosacea:0.54), (ETORICOXIB:Rosacea:0.22), (EVOLOCUMAB:Rosacea:1.48), (FEX OFENADINEPSEUDOEPHEDRINE:Rosacea:0.15), (FLUVOXAMINE MALEATE:Rosacea:0), (FORMOTEROL FUMARATE:Rosacea:0.26), (GLICLAZIDE:Rosacea:0.21), (GLYCOPYRRONIUMINDACAT EROL:Rosacea:0.02), (HALOPERIDOL:Rosacea:0.04), (HYALURONATE SODIUM:Rosacea:0.13), (HYDROCHLOROTHIAZIDEIRBESARTAN:Rosacea:0.39), (HYDR OCODONE BITARTRATEIBUPROFEN:Rosacea:0.17), (HYDROMORPHONE:Rosacea:0.79), (IMATINIB MESYLATE:Rosacea:0.25), (IMIPRAMINE HYDROCHLORIDE:Rosacea:0.66), (INDAPAMIDE:Rosacea:0.39), (INDAPAMIDEPERIND OPRIL:Rosacea:0.15), (INFLIXIMAB-DYYB:Rosacea:0.48), (INSULIN HUMAN:Rosacea:0.3), (IPRATROPIUM:Rosacea:0.98), (ISOSORBIDE:Rosacea:0.7), (LABET ALOLLABETALOL HYDROCHLORIDE:Rosacea:0.67), (LACOSAMIDE:Rosacea:0.03), (LACTASE:Rosacea:0.46), (LERCANIDIPINE:Rosacea:0.25), (LERCANIDIPINE HYDROCHLORIDE:Rosacea:0.36), (LEVOMEFOLATE CALCIUM:Rosacea:0.29), (LITHIUM:Rosacea:0.31), (LOMITAPIDE MESYLATE:Rosacea:0.24), (METHYLPREDNISOLONE ACETATE:Rosacea:1.2), (MOXIFLOXACIN HYDROCHLORIDE:Rosacea:1.68), (NALOXONE HYDROCHLORIDEOXYCODONE HYDROCHLORIDE:Rosacea:0.58), (NATEGLINIDE:Rosacea:0.09), (NILOTINIB:Rosacea:0.41), (NORTRIPTYLINE HYDROCHLORIDE:Rosacea:0.18), (OXCARBAZEPINE:Rosacea:1.76), (PANTOPRAZOLE:R osacea:2.02), (PANTOPRAZOLE MAGNESIUM:Rosacea:1.38), (PAZOPANIB HYDROCHLORIDE:Rosacea:0.26), (PEGINTERFERON BETA-1A:Rosacea:0.24), (PEMETREXED DISODIUM:Rosacea:0.11), (PENICILLIN:Rosacea:0.8), (PHENOBARBITAL:Rosacea:0.53), (PI ROXICAM:Rosacea:0.26), (PONATINIB:Rosacea:0.23), (PRAZOSIN:Rosacea:0.03), (PROPAF ENONE HYDROCHLORIDE:Rosacea:0), (PSYLLIUM HUSK:Rosacea:0.22), (PYRIDOXINE:Rosacea:0.17), (RANOLAZINE:Rosacea:1.33), (RIZATRI PTAN:Rosacea:0.15), (ROFLUMILAST:Rosacea:0.58), (SACUBITRILVALSARTAN:Rosacea: 0.4), (SULFAMETHOXAZOLE:Rosacea:0.49), (SUNITINIB MALATE:Rosacea:0.08), (TERBINAFINE HYDROCHLORIDE:Rosacea:0.64), (TERBUTALINE SULFATE:Rosacea:0.4), (UMECLIDINIUM BROMIDEVILANTEROL TRIFENATATE:Rosacea:1.34), (ZAFIRLUKAST:Rosacea:0.11), (DEXLANSOPRAZOLE:Rota tor cuff syndrome:0.04), (.ALPHA.1-PROTEINASE INHIBITOR HUMAN:Routine health maintenance:0.42), (ACETAMINOPHENPROPOXYPHENE HYDROCHLORIDE:Routine health maintenance:0.24), (ACETAMINOPHENPROPOXYPHENE NAPSYLATE:Routine health maintenance:0.34), (ADO-TRASTUZUMAB EMTANSINE:Routine health maintenance:0.79), (AGALSIDASE BETA:Routine health maintenance:0.58), (ALBIGLUTIDE:Routine health maintenance:0.51), (ALCOHOL:Routine health maintenance:0.54), (ALENDRONIC ACID:Routine health maintenance:0.71), (ALFACALCIDOL:Routine health maintenance:0.34), (AMINO ACIDSDEXTROSEELECTROLYTES NOS:Routine health maintenance:0.36), (AMISULPRIDE:Routine health maintenance:0.53), (AMLODIPINE BESYLATEHYDROCHLOROTHIAZIDEVALSARTAN:Routine health maintenance:0.3), (AMMONIUM CHLORIDEDEXTROMETHORPHAN\SODIUM CITRATE:Routine health maintenance:0.08), (ANTIHISTAMINES NOS:Routine health maintenance:0.46), (ASPIRIN LYSINE:Routine health maintenance:0.2), (ASPIRINBUTALBITALCAFFEINE:Routine health maintenance:0.04), (BALSALAZIDE DISODIUM:Routine health maintenance:0.23), (BETAMETHASONE DIPROPIONATECALCIPOTRIENECALCIPOTRIENE HYDRATE:Routine health maintenance:0.58), (BETAMETHASONE DIPROPIONATECLOTRIMAZOLE:Routine health maintenance:0.22), (BETHANECHOL CHLORIDE:Routine health maintenance:0.03), (BLINATUMOMAB:Routine health maintenance:0.15), (BUTYLSCOPOLAMINE BROMIDE:Routine health maintenance:0.38), (CABOZANTINIB S-MALATE:Routine health maintenance:0.06), (CALCITONIN:Routine health maintenance:0.44), (CANAKINUMAB:Routine health maintenance:0.17), (CANDESARTAN CILEXETILHYDROCHLOROTHIAZIDE:Routine health maintenance:1.64), (CARBIDOPA:Routine health maintenance:0.04), (CARVEDILOL PHOSPHATE:Routine health maintenance:1.99), (CEFDINIR:Routine health maintenance:0.1), (CEFOTAXIME SODIUM:Routine health maintenance:0.01), (CEFTAZIDIME:Routine health maintenance:0.08), (CEFUROXIME:Routine health maintenance:0.3), (CEVIMELINE HYDROCHLORIDE:Routine health maintenance:0.18), (CHLORPHENIRAMINEHYDROCODONE:Routine health maintenance:0.32), (CHLORPROMAZINE:Routine health maintenance:0.03), (CIPROFIBRATE:Routine health maintenance:0.33), (CLOMIPRAMINE HYDROCHLORIDE:Routine health maintenance:0.01), (CLOZAPINE:Routine health maintenance:0.7), (CODEINE PHOSPHATEGUAIFENESIN:Routine health maintenance:0.43), (CORTICOTROPIN:Routine health maintenance:0.79), (CYPROHEPTADINE:Routine health maintenance:0.68), (DACLIZUMAB:Routine health maintenance:1.31), (DANAZOL:Routine health maintenance:0.53), (DASATINIB:Routine health maintenance:1.71), (DESLORATADINE:Routine health maintenance:0.98), (DEXTROAMPHETAMINE:Routine health maintenance:0.04), (DEXTROAMPHETAMINE SULFATE:Routine health maintenance:0.56), (DIFLUNISAL:Routine health maintenance:0.09), (DIGITOXIN:Routine health maintenance:0.04), (DIPYRIDAMOLE:Routine health maintenance:0.27), (DOCETAXEL:Routine health maintenance:0.57), (DOLASETRON MESYLATE:Routine health maintenance:0.42), (DORZOLAMIDE:Routine health maintenance:0.74), (DROSPIRENONEETHINYL ESTRADIOL:Routine health maintenance:0.03), (DROXIDOPA:Routine health maintenance:0.13), (DUTASTERIDETAMSULOSIN HYDROCHLORIDE:Routine health maintenance:0.51), (ECONAZOLE:Routine health maintenance:0.08), (EDOXABAN TOSYLATE:Routine health maintenance:1.07), (ELOTUZUMAB:Routine health maintenance:0.16), (EMPAGLIFLOZINMETFORMIN HYDROCHLORIDE:Routine health maintenance:0.2), (EPIRUBICIN:Routine health maintenance:0.42), (ESOMEPRAZOLE MAGNESIUMNAPROXEN:Routine health maintenance:0.62), (ETHINYL ESTRADIOLNORGESTREL:Routine health maintenance:0.01), (ETORICOXIB:Routine health maintenance:0.15), (EVOLOCUMAB:Routine health maintenance:1.9), (FLUVASTATIN SODIUM:Routine health maintenance:0.03), (FORMOTEROL:Routine health maintenance:0.35), (FORMOTEROL FUMARATE\MOMETASONE FUROATE:Routine health maintenance:0.42), (FULVESTRANT:Routine health maintenance:0.42), (GENTAMICIN:Routine health maintenance:0), (GIMERACILOTERACILTEGAFUR:Routine health maintenance:0.2), (GLIPIZIDEMETFORMIN HYDROCHLORIDE:Routine health maintenance:0.22), (GLYCOPYRRONIUMINDACATEROL:Routine health maintenance:0.86), (GUANFACINE:Routine health maintenance:0.41), (HYDROCODONE BITARTRATEIBUPROFEN:Routine health maintenance:0.69), (HYDROXYCHLOROQUINE DIPHOSPHATE:Routine health maintenance:0.39), (ILOPROST:Routine health maintenance:0.74), (IMATINIB MESYLATE:Routine health maintenance:1.17), (IMIPRAMINE:Routine health maintenance:0.28), (IMIPRAMINE HYDROCHLORIDE:Routine health maintenance:1.3), (INDACATEROL:Routine health maintenance:0.13), (INSULIN BEEF:Routine health maintenance:0.13), (INSULIN DEGLUDEC:Routine health maintenance:0.77), (INTERFERON:Routine health maintenance:0.14), (INTERFERON ALFA-2A:Routine health maintenance:0.34), (ISOSORBIDE DINITRATE:Routine health maintenance:0.33), (IXAZOMIB:Routine health maintenance:0.48), (KETOPROFEN:Routine health maintenance:0.08), (KETOROLACKETOROLAC TROMETHAMINE:Routine health maintenance:0.89), (LACOSAMIDE:Routine health maintenance:0.39), (ACIDOPHILUSLACTOBACILLUS DELBRUECKII SUBSP. BULGARICUS:Routine health maintenance:0.09), (LAPATINIB DITOSYLATE:Routine health maintenance:0.39), (LENVATINIB:Routine health maintenance:0.02), (LERCANIDIPINE:Routine health maintenance:1.21), (LERCANIDIPINE HYDROCHLORIDE:Routine health maintenance:0.63), (LEUCOVORIN CALCIUM:Routine health maintenance:0.57), (LIOTHYRONINE:Routine health maintenance:0.7), (LOMITAPIDE MESYLATE:Routine health maintenance:1.01), (MEFENAMIC ACID:Routine health maintenance:0.24), (MEPOLIZUMAB:Routine health maintenance:0.18), (METAMIZOLE:Routine health maintenance:0.16), (METFORMIN HYDROCHLORIDEROSIGLITAZONE MALEATE:Routine health maintenance:0.3), (METFORMIN HYDROCHLORIDEVILDAGLIPTIN:Routine health maintenance:0.46), (METHYLPREDNISOLONE ACETATE:Routine health maintenance:0.94), (MOEXIPRIL HYDROCHLORIDE:Routine health maintenance:0.32), (MORPHINE SULFATE\NALTREXONE HYDROCHLORIDE:Routine health maintenance:0.33), (MUPIROCINMUPIROCIN CALCIUM:Routine health maintenance:1.08), (NALOXEGOL OXALATE:Routine health maintenance:0.14), (NALOXONE:Routine health maintenance:0.11), (NARATRIPTAN HYDROCHLORIDE:Routine health maintenance:0.06), (NEBIVOLOL:Routine health maintenance:0.55), (NILOTINIB:Routine health maintenance:1.91), (NOREPINEPHRINE BITARTRATE:Routine health maintenance:0.11), (NORTRIPTYLINE HYDROCHLORIDE:Routine health maintenance:0.69), (OLOPATADINE:Routine health maintenance:0.03), (OMEPRAZOLE SODIUM:Routine health maintenance:0.19), (OSELTAMIVIR PHOSPHATE:Routine health maintenance:0.16), (OSPEMIFENE:Routine health maintenance:0.79), (OXYBUTYNINOXYBUTYNIN CHLORIDE:Routine health maintenance:0.38), (PAPAYA:Routine health maintenance:0.11), (PATIROMER:Routine health maintenance:0.55), (PAZOPANIB HYDROCHLORIDE:Routine health maintenance:1.15), (PEMETREXED DISODIUM:Routine health maintenance:0.71), (PENICILLIN G:Routine health maintenance:0.04), (PENICILLIN V:Routine health maintenance:0.18), (PENTOXIFYLLINE:Routine health maintenance:0.31), (PERTUZUMAB:Routine health maintenance:0.17), (PHENYTOIN SODIUM:Routine health maintenance:0.19), (PILOCARPINE:Routine health maintenance:0.48), (PIMECROLIMUS:Routine health maintenance:0.66), (PIRFENIDONE:Routine health maintenance:0.15), (PREDNISOLONE SODIUM PHOSPHATE:Routine health maintenance:0.17), (PREDNISOLONEPREDNISOLONE ACETATEPREDNISOLONE SODIUM SUCCINATE:Routine health maintenance:0.12), (PROTRIPTYLINE HYDROCHLORIDE:Routine health maintenance:0.39), (RADIATION THERAPY:Routine health maintenance:0.92), (REGORAFENIB:Routine health maintenance:0.83), (RIZATRIPTAN:Routine health maintenance:0.5), (SACUBITRILVALSARTAN:Routine health maintenance:0.8), (SALMETEROL:Routine health maintenance:0.06), (SECUKINUMAB:Routine health maintenance:0.8), (SELEGILINE:Routine health maintenance:0.15), (SELEXIPAG:Routine health maintenance:0.48), (SILVER SULFADIAZINE:Routine health maintenance:0.24), (SODIUM PHOSPHATE, DIBASIC\SODIUM PHOSPHATE, MONOBASIC:Routine health maintenance:0.35), (SULINDAC:Routine health maintenance:0.18), (TEDUGLUTIDE\WATER:Routine health maintenance:0.24), (TELOTRISTAT ETHYL:Routine health maintenance:0.11), (TERBUTALINE SULFATE:Routine health maintenance:1.04), (TESTOSTERONE UNDECANOATE:Routine health maintenance:0.06), (TETRACYCLINE:Routine health maintenance:1.63), (TINZAPARIN SODIUM:Routine health maintenance:0.31), (TIPIRACIL HYDROCHLORIDETRIFLURIDINE:Routine health maintenance:0.09), (TOCILIZUMAB:Routine health maintenance:0.18), (TRIPTORELIN PAMOATE:Routine health maintenance:0.07), (VEMURAFENIB:Routine health maintenance:0.17), (VINCRISTINE SULFATE:Routine health maintenance:0.72), (ZILEUTON:Routine health maintenance:0.03), (BUPRENORPHINE HYDROCHLORIDENALOXONE HYDROCHLORIDE:Salivary hypersecretion:0.02), (LACOSAMIDE:Salivary hypersecretion:0.32), (MORPHINE:Salivary hypersecretion:0.02), (TERIFLUNOMIDE:Salivary hypersecretion:0.19), (VARENICLINE TARTRATE:Salivary hypersecretion:0.4), (THIOGUANINE:Salvage therapy:0.03), (BISACODYL OR DOCUSATE SODIUM:Sarcoidosis:0.38), (CALCITRIOL:Sarcoidosis:0.31), (CALCIUM ACETATE:Sarcoidosis:0.5), (CALCIUM CITRATE:Sarcoidosis:0.24), (CANDESARTAN CILEXETILHYDROCHLOROTHIAZIDE:Sarcoidosis:0.28), (CARFILZOMIB:Sarcoidosis:0.31), (CARVEDILOL PHOSPHATE:Sarcoidosis:0.52), (CEFTRIAXONE SODIUM:Sarcoidosis:0.18), (CHOLESTYRAMINE:Sarcoidosis:0.78), (CLOZAPINE:Sarcoidosi s:0.29), (DACLIZUMAB:Sarcoidosis:0.36), (DAPAGLIFLOZIN PROPANEDIOL:Sarcoidosis:0), (DASATINIB:Sarcoidosis:0.61), (DICYCLOMINE HYDROCHLORIDE:Sarcoidosis:0.49), (DIETARY SUPPLEMENT:Sarcoidosis:0.1), (DOCUSATE:Sarcoidosis:0.19), (DOFETILIDE:Sarcoidosis:0.08), (DOXAZOSIN MESYLATE:Sarcoidosis:0.01), (DOXAZOSINDOXAZOSIN MESYLATE:Sarcoidosis:0.1), (DUTASTERIDE:Sarcoidosis:0.3), (ELETRIPTAN HYDROBROMIDE:Sarcoidosis:0.65), (ENALAPRIL MALEATE:Sarcoidosis:0.41), (EPINEPHRINE:Sarcoidosis:0.61), (ESZOPICLONE:Sarcoidosis: 0.76), (EVEROLIMUS:Sarcoidosis:0.37), (EVOLOCUMAB:Sarcoidosis:1.04), (FERROUS SULFATE:Sarcoidosis:1.08), (FINGOLIMOD HYDROCHLORIDE:Sarcoidosis:0.62), (FLUTICASONE FUROATE:Sarcoidosis:0.02), (FORMOTEROL FUMARATE\MOMETASONE FUROATE:Sarcoidosis:0.11), (GLYCOPYRRONIUMINDACATEROL:Sarcoidosis:0.15), (HY DROCHLOROTHIAZIDEIRBESARTAN:Sarcoidosis:0.13), (HYDROXYZINE PAMOATE:Sarcoidosis:0.54), (HYOSCYAMINE:Sarcoidosis:0.07), (IBRUTINIB:Sarcoidosis:1.17), (IMIPRAMINE HYDROCHLORIDE:Sarcoidosis:0.41), (LEDIPASVIRSOFOSBUVIR:Sarcoidosis:0.56), (LER CANIDIPINE:Sarcoidosis:0.42), (LIDOCAINEPRILOCAINE:Sarcoidosis:0.03), (METAXALO NE:Sarcoidosis:0.68), (METHYLPREDNISOLONE ACETATE:Sarcoidosis:0.18), (METRONIDAZOLEMETRONIDAZOLE HYDROCHLORIDE:Sarcoidosis:0.27), (NALOXONE HYDROCHLORIDEOXYCODONE HYDROCHLORIDE:Sarcoidosis:0.05), (NAPROXEN SODIUM:Sarcoidosis:0.45), (NEBIVOLOL:Sarcoidosis:0.2), (OXCARBAZEPINE:Sarcoidosis:10.13), (PANTOPRAZOLE MAGNESIUM:Sarcoidosis:1.06), (PEGINTERFERON BETA-1A:Sarcoidosis:0.19), (PHENOBARBITAL:Sarcoidosis:0.15), (PONATINIB:Sarcoidosis:0.12), (PRAVASTATIN SODIUM:Sarcoidosis:0.86), (PROBIOTICS NOS:Sarcoidosis:0.42), (PYRAZINAMIDE:Sarcoidosis:0.69), (QUETIAPINE:Sarcoidosis:0.38), (RABEPRAZOLE:Sarcoidosis:0.4), (RANOLAZINE:Sarcoidosis:1.21), (ROPINIROLE HYDROCHLORIDE:Sarcoidosis:0.14), (SACUBITRILVALSARTAN:Sarcoidosis:0.94), (SUM ATRIPTANSUMATRIPTAN SUCCINATE:Sarcoidosis:0.55), (TIMOLOL:Sarcoidosis:0.45), (TOCILIZUMAB:Sarcoidosis:0.14), (TOLTERODINE TARTRATE:Sarcoidosis:0.12), (VALSARTAN:Sarcoidosis:1.3), (VILAZODONE HYDROCHLORIDE:Sarcoidosis:0.03), (ZINC:Sarcoidosis:0.05), (ANASTROZOLE:Sarcoma:0.36), (BUMETANIDE:Sarcoma:0.17), (DASATINIB:Sarcoma:0.46), (DICLOFENAC SODIUM:Sarcoma:0.05), (DILTIAZEM:Sarcoma:0.23), (DILTIAZEM HYDROCHLORIDE:Sarcoma:0.03), (HYDROXYUREA:Sarcoma:0.03), (METRONIDAZOLE: Sarcoma:0.08), (NEBIVOLOL:Sarcoma:0.14), (NILOTINIB:Sarcoma:0.42), (PANTOPRAZOLE: Sarcoma:0.52), (METRONIDAZOLE:Scan with contrast:0.06), (WARFARIN SODIUM:Scan with contrast:0.04), (ACETAMINOPHENBUTALBITALCAFFEINE:Schizoaffective disorder:0.75), (ACETAMINOPHENOXYCODONEOXYCODONE HYDROCHLORIDE:Schizoaffective disorder:0.23), (AMIODARONE:Schizoaffective disorder:0.58), (AMOBARBITAL SODIUM:Schizoaffective disorder:0.03), (AMPHETAMINE SULFATE:Schizoaffective disorder:0.05), (BLONANSERIN:Schizoaffective disorder:0.4), (BUMETANIDE:Schizoaffective disorder:0.27), (BUPRENORPHINE HYDROCHLORIDE:Schizoaffective disorder:2.52), (BUPRENORPHINENALOXONE:Schizoaffective disorder:0.74), (CALCIUM ACETATE:Schizoaffective disorder:0.24), (CANDESARTAN CILEXETILHYDROCHLOROTHIAZIDE:Schizoaffective disorder:0.53), (CARVEDILOL PHOSPHATE:Schizoaffective disorder:0.15), (CHLORTHALIDONE:Schizoaffective disorder:0.19), (CHOLESTYRAMINE:Schizoaffective disorder:0.45), (CIMETIDINE:Schizoaffective disorder:0.06), (CLOMETHIAZOLE:Schizoaffective disorder:0.09), (CORTISONEHYDROCORTISONE:Schizoaffective disorder:0.25), (CYPROHEPTADINE:Schizoaffective disorder:0.13), (DAPAGLIFLOZIN PROPANEDIOL:Schizoaffective disorder:0.17), (DASATINIB:Schizoaffective disorder:0.08), (DESLORATADINE:Schizoaffective disorder:0.47), (EDOXABAN TOSYLATE:Schizoaffective disorder:0.1), (ERYTHROPOIETIN:Schizoaffective disorder:0.12), (ETHAMBUTOL HYDROCHLORIDE:Schizoaffective disorder:0.9), (EVEROLIMUS:Schizoaffective disorder:0.03), (EVOLOCUMAB:Schizoaffective disorder:0.81), (EZETIMIBESIMVASTATIN:Schizoaffective disorder:0.19), (FLECAINIDE:Schizoaffective disorder:0.59), (GLYCOPYRRONIUMINDACATEROL:Schizoaffective disorder:0.01), (HYDROCHLOROTHIAZIDEVALSARTAN:Schizoaffective disorder:0.21), (HYDROMORPHONE:Schizoaffective disorder:0.19), (HYDROMORPHONE HYDROCHLORIDE:Schizoaffective disorder:0.55), (IBRUTINIB:Schizoaffective disorder:0.17), (INDOMETHACIN:Schizoaffective disorder:0.23), (ISOSORBIDE:Schizoaffective disorder:0.37), (LABETALOLLABETALOL HYDROCHLORIDE:Schizoaffective disorder:0.79), (LEDIPASVIRSOFOSBUVIR:Schizoaffective disorder:0.43), (LERCANIDIPINE:Schizoaffective disorder:0.2), (LERCANIDIPINE HYDROCHLORIDE:Schizoaffective disorder:0.15), (LEVALBUTEROL HYDROCHLORIDE:Schizoaffective disorder:0.05), (LINAGLIPTIN:Schizoaffective disorder:0.32), (LISDEXAMFETAMINE:Schizoaffective disorder:0.06), (LYSERGIDE:Schizoaffective disorder:0.19), (METAXALONE:Schizoaffective disorder:0.71), (METHYLPREDNISOLONE ACETATE:Schizoaffective disorder:0.2), (METHYLPREDNISOLONE SODIUM SUCCINATE:Schizoaffective disorder:0.6), (MOXIFLOXACIN HYDROCHLORIDE:Schizoaffective disorder:1), (NAPROXEN SODIUM:Schizoaffective disorder:0.24), (NEBIVOLOL:Schizoaffective disorder:0.19), (NIACIN:Schizoaffective disorder:0.3), (NILOTINIB:Schizoaffective disorder:0.53), (OXYMORPHONE:Schizoaffective disorder:0.11), (PANTOPRAZOLE MAGNESIUM:Schizoaffective disorder:0.17), (PENICILLIN:Schizoaffective disorder:0.65), (PERAMPANEL:Schizoaffective disorder:0.55), (PEROSPIRONE:Schizoaffective disorder:0.45), (PHENOBARBITAL:Schizoaffective disorder:0.95), (PIMAVANSERIN TARTRATE:Schizoaffective disorder:0.03), (PRAMIPEXOLE:Schizoaffective disorder:1.6), (PROMAZINE HYDROCHLORIDE:Schizoaffective disorder:0.15), (RANOLAZINE:Schizoaffective disorder:1.07), (RISEDRONATE SODIUM:Schizoaffective disorder:0.12), (RIVASTIGMINE TARTRATE:Schizoaffective disorder:0.47), (SACUBITRILVALSARTAN:Schizoaffective disorder:0.23), (SECUKINUMAB:Schizoaffective disorder:0.64), (SELEGILINE:Schizoaffective disorder:0.42), (SERTINDOLE:Schizoaffective disorder:0.23), (SUMATRIPTAN SUCCINATE:Schizoaffective disorder:0.06), (TERIFLUNOMIDE:Schizoaffective disorder:0.5), (THALIDOMIDE:Schizoaffective disorder:0.16), (TIAPRIDE:Schizoaffective disorder:0.27), (TOLTERODINE TARTRATE:Schizoaffective disorder:0.23), (TRIAMCINOLONE ACETONIDE:Schizoaffective disorder:0.43), (UMECLIDINIUM BROMIDEVILANTEROL TRIFENATATE:Schizoaffective disorder:0.18), (PHENOBARBITAL:Schizoaffective disorder bipolar type:0.11), (ZONISAMIDE:Schizoaffective disorder bipolar type:0.28), (ABIRATERONE ACETATE:Schizophrenia:0.22), (ACETAMINOPHENDIPHENHYDRAMINE:Schizophrenia:0), (ACETAMINOPHENOXYCODONEOXYCODONE HYDROCHLORIDE:Schizophrenia:1.24), (ACETAMINOPHENPROPOXYPHENE HYDROCHLORIDE:Schizophrenia:0.01), (ACTIVATED CHARCOAL:Schizophrenia:0.78), (ADO-TRASTUZUMAB EMTANSINE:Schizophrenia:0.52), (AGALSIDASE BETA:Schizophrenia:0.49), (ALBUTEROLIPRATROPIUM:Schizophrenia:0.17), (ALEMTUZ UMAB:Schizophrenia:0.45), (ALISKIREN:Schizophrenia:0.37), (AMANTADINE SULFATE:Schizophrenia:0.14), (AMIKACIN SULFATE:Schizophrenia:0.04), (AMLODIPINE BESYLATEHYDROCHLOROTHIAZIDEVALSARTAN:Schizophrenia:0.29), (AMLODIPIN EPERINDOPRIL:Schizophrenia:0.37), (AMLODIPINEVALSARTAN:Schizophrenia:0.75), (A MPHETAMINE ADIPATEDEXTROAMPHETAMINE:Schizophrenia:0.29), (AMPHETAMINE SULFATE:Schizophrenia:0.39), (AMPHETAMINEDEXTROAMPHETAMINE:Schizophrenia: 0.02), (AMPICILLIN:Schizophrenia:1.17), (ASUNAPREVIR:Schizophrenia:0.48), (ATOVAQU ONE:Schizophrenia:0.34), (AZELASTINE:Schizophrenia:0), (AZELASTINE HYDROCHLORIDE:Schizophrenia:0.17), (BENDAMUSTINE HYDROCHLORIDE:Schizophrenia:0.35), (BEPRIDIL:Schizophrenia:0.27), (BERAPROST SODIUM:Schizophrenia:0.62), (BETAHISTINE MESILATE:Schizophrenia:0.94), (BETAMETHASONECALCIPOTRIENE:Schizophrenia:0.19), (BIFIDOBACTERIUM LONGUM SUBSP. INFANTIS:Schizophrenia:0.15), (BILASTINE:Schizophrenia:0.65), (BOTULINUM TOXIN TYPE A:Schizophrenia:0.21), (BRIVARACETAM:Schizophrenia:0.14), (BUPRENORPHINENALOX ONE:Schizophrenia:1.19), (CALCIUM CARBONATEVITAMIN D:Schizophrenia:0.49), (CALCIUM\MAGNESIUM:Schizophrenia:0), (CALCIUMMAGNESIU M\ZINC:Schizophrenia:0.16), (CANAGLIFLOZINMETFORMIN HYDROCHLORIDE:Schizophrenia:0.87), (CAPSAICIN:Schizophrenia:0.12), (CARBIDOPA:Sc hizophrenia:1.22), (CARBIDOPAMELEVODOPA:Schizophrenia:0.46), (CARVEDILOL PHOSPHATE:Schizophrenia:1.12), (CEFAZOLIN SODIUM:Schizophrenia:1.01), (CEFOTAXIME SODIUM:Schizophrenia:0.56), (CELERY SEED:Schizophrenia:0.26), (CELIPROLOL:Schizophrenia:0.04), (CETIRIZINE HYDROCHLORIDEPSEUDOEPHEDRINE HYDROCHLORIDE:Schizophrenia:0.01), (CHOLESTYRAMINE:Schizophrenia:1.77), (CITIC OLINE SODIUM:Schizophrenia:0.13), (CLEMASTINE FUMARATE:Schizophrenia:0.02), (CLOXACILLIN SODIUM:Schizophrenia:0.04), (CODEINE PHOSPHATEGUAIFENESIN:Schizophrenia:0.09), (CORTICOTROPIN:Schizophrenia:0.5), (D ABIGATRAN:Schizophrenia:0.85), (DACLIZUMAB:Schizophrenia:0.68), (DEFLAZACORT:Sc hizophrenia:1.12), (DEGARELIX:Schizophrenia:0.26), (DESONIDE:Schizophrenia:0.65), (DEUT ETRABENAZINE:Schizophrenia:0.22), (DEVICE:Schizophrenia:0.64), (DEXAMETHASONE ACETATE:Schizophrenia:0.94), (DEXAMETHASONE SODIUM PHOSPHATE:Schizophrenia:0.74), (DEXMETHYLPHENIDATE HYDROCHLORIDE:Schizophrenia:0.54), (DEXTROAMPHETAMINE:Schizophrenia:0.45), (D IGITOXIN:Schizophrenia:0.34), (DILAZEP:Schizophrenia:0.16), (DIPHENIDOL HYDROCHLORIDE:Schizophrenia:0.33), (DOFETILIDE:Schizophrenia:0.53), (DRONEDARO NE:Schizophrenia:0.03), (DUTASTERIDETAMSULOSIN HYDROCHLORIDE:Schizophrenia:0.67), (ECONAZOLE:Schizophrenia:0.82), (EDARAVONE: Schizophrenia:0.18), (EDOXABAN TOSILATE MONOHYDRATE:Schizophrenia:0.41), (EDOXABAN TOSYLATE:Schizophrenia:1.67), (ELETRIPTAN HYDROBROMIDE:Schizophrenia:1.69), (ELOTUZUMAB:Schizophrenia:0.14), (ELTROMBO PAG OLAMINE:Schizophrenia:0.14), (EMPAGLIFLOZINLINAGLIPTIN:Schizophrenia:0.28), (EM TRICITABINERILPIVIRINE HYDROCHLORIDETENOFOVIR DISOPROXIL FUMARATE:Schizophrenia:0.02), (EMTRICITABINERILPIVIRINETENOFOVIR DISOPROXIL FUMARATE:Schizophrenia:0.09), (ENALAPRIL MALEATEHYDROCHLOROTHIAZIDE:Schizophrenia:0.73), (ERTAPENEM SODIUM:Schizophrenia:0.14), (ERYTHROMYCIN LACTOBIONATE:Schizophrenia:0.26), (ESMOLOL HYDROCHLORIDE:Schizophrenia:0.49), (ESTRIOL:Schizophrenia:0.03), (ESTRONE\SUS SCROFA OVARY:Schizophrenia:0.67), (ETHINYL ESTRADIOLNORETHINDRONE ACETATE:Schizophrenia:0.02), (ETHINYL ESTRADIOLNORGESTREL:Schizophrenia:0.11), (ETHYL ICOSAPENTATEICOSAPENT:Schizophrenia:0.03), (ETRETINATE:Schizophrenia:0.48), (EV OLOCUMAB:Schizophrenia:1.04), (FEXOFENADINEPSEUDOEPHEDRINE:Schizophrenia:00.3), (FISH OIL\OMEGA-3 FATTY ACIDS:Schizophrenia:0.23), (FLUMAZENIL:Schizophrenia:0.17), (FLUOCINONIDE:Schizoph renia:0.45), (FORMOTEROL FUMARATE\MOMETASONE FUROATE:Schizophrenia:0.46), (FOSPHENYTOIN SODIUM:Schizophrenia:0.19), (GIMERACILOTERACILTEGAFUR:Schizophrenia:0.49), (GL YCOPYRROLATEINDACATEROL MALEATE:Schizophrenia:0.4), (GLYCOSAMINOGLYCANS:Schizophrenia:0.31), (HERBALS SAW PALMETTO:Schizophrenia:0.55), (HERBALSTURMERIC:Schizophrenia:0.33), (HYDROCHL OROTHIAZIDETELMISARTAN:Schizophrenia:1.08), (HYDROCORTISONE ACETATE:Schizophrenia:0.42), (HYDROCORTISONE SODIUM SUCCINATE:Schizophrenia:0.48), (HYDROXYCHLOROQUINE DIPHOSPHATE:Schizophrenia:0.22), (HYDROXYUREA:Schizophrenia:0.6), (HYOSCYAMIN E:Schizophrenia:0.2), (HYPERICUM PERFORATUM:Schizophrenia:0.62), (ILOPROST:Schizophrenia:0.1), (IMIGLUCERASE:Schiz ophrenia:0.12), (INSULIN ASPARTINSULIN DEGLUDEC:Schizophrenia:0.13), (ISONIAZIDRIFAMPIN:Schizophrenia:0.34), (IXAZOMIB: Schizophrenia:0.36), (LABETALOL HYDROCHLORIDE:Schizophrenia:1.24), (LACTOBACILLUS RHAMNOSUS:Schizophrenia:0.48), (LEVALBUTEROL:Schizophrenia:0.01), (LEVALBUTEROL HYDROCHLORIDE:Schizophrenia:0.89), (LIOTHYRONINE:Schizophrenia:0.25), (LISDEXA MFETAMINE:Schizophrenia:0.7), (LITHIUM SULFATE:Schizophrenia:1.94), (MAPROTILINE:Schizophrenia:0.06), (MARAVIROC:Schizop hrenia:0.06), (MEGESTROL ACETATE:Schizophrenia:0.33), (MEPERIDINE HYDROCHLORIDE:Schizophrenia:0.7), (MEPOLIZUMAB:Schizophrenia:0.36), (MESNA:Schi zophrenia:0.13), (METHENAMINE HIPPURATE:Schizophrenia:1.1), (METHSUXIMIDE:Schizophrenia:0.06), (METHYLPREDNIS OLONE ACETATE:Schizophrenia:1.26), (METRELEPTIN:Schizophrenia:0.03), (MIDOMAFETAMINE: Schizophrenia:0.01), (MIGLUSTAT:Schizophrenia:0.61), (MINODRONIC ACID:Schizophrenia:0.35), (MITIGLINIDE CALCIUM HYDRATEVOGLIBOSE:Schizophrenia:0.3), (MIZORIBINE:Schizophrenia:0.19), (MOEXIPRI L HYDROCHLORIDE:Schizophrenia:0.26), (MORPHINE SULFATE\NALTREXONE HYDROCHLORIDE:Schizophrenia:0.62), (NADOLOL:Schizophrenia:0.65), (NALOXONE HYDROCHLORIDETILIDINE HYDROCHLORIDE:Schizophrenia:0.89), (NEOSTIGMINE:Schizophrenia:0.2), (NIAOULI OILQUININE BENZOATETHIAMINE HYDROCHLORIDE:Schizophrenia:0.35), (NINTEDANIB:Schizophrenia:0.59), (NIRAPARIB:S chizophrenia:0.22), (NITROFURANTOINNITROFURANTOIN MONOHYDRATE:Schizophrenia:0.66), (OLAPARIB:Schizophrenia:0.07), (ONABOTULINUM TOXINA:Schizophrenia:0.67), (OSELTAMIVIR:Schizophrenia:0.36), (OSPEMIFENE:Schizoph renia:0.03), (PAMIDRONATE DISODIUM:Schizophrenia:0.17), (PANITUMUMAB:Schizophrenia:0.39), (PEGINTERFERON BETA-1A:Schizophrenia:0.62), (PEMETREXED:Schizophrenia:0.46), (PERAMPANEL:Schizophrenia: 1.25), (PHENELZINE:Schizophrenia:0.53), (PHENOBARBITAL SODIUM:Schizophrenia:0.58), (PILOCARPINE:Schizophrenia:0.18), (PIMOBENDAN:Schizop hrenia:0.37), (PIRIBEDIL:Schizophrenia:0.99), (POTASSIUM IODIDE:Schizophrenia:0.1), (PREDNISOLONE ACETATE:Schizophrenia:0.95), (PREDNISOLONE SODIUM PHOSPHATE:Schizophrenia:0.59), (PREDNISOLONE SODIUM SUCCINATE:Schizophrenia:0.09), (PROPAFENONE:Schizophrenia:0.38), (PROTRIPTYLINE HYDROCHLORIDE:Schizophrenia:0.13), (QUINAGOLIDE HYDROCHLORIDE:Schizophrenia:0.12), (RAMUCIRUMAB:Schizophrenia:0.16), (RASAGILI NE:Schizophrenia:1.03), (RASAGILINE MESYLATE:Schizophrenia:1.35), (REGORAFENIB:Schizophrenia:0.26), (RILPIVIRINE HYDROCHLORIDE:Schizophrenia:0.56), (SACCHAROMYCES CEREVISIAE:Schizophrenia:0.16), (SAFINAMIDE:Schizophrenia:0.22), (SAFINAMIDE MESYLATE:Schizophrenia:0.48), (SARPOGRELATE:Schizophrenia:1.09), (SARPOGRELATE HYDROCHLORIDE:Schizophrenia:0.78), (SENNOSIDE B:Schizophrenia:0.27), (SODIUM CITRATE:Schizophrenia:0.05), (SOTALOL HYDROCHLORIDE:Schizophrenia:0.21), (ST. JOHN'S WORT:Schizophrenia:0.33), (SUFENTANIL CITRATE:Schizophrenia:0.44), (SULTHIAME:Schizophrenia:0.15), (SUPLATAST TOSILATE:Schizophrenia:0.81), (TAZOBACTAM:Schizophrenia:0.2), (TEDUGLUTIDEWAT ER:Schizophrenia:0.06), (TEMOCAPRIL HYDROCHLORIDE:Schizophrenia:0.11), (TERAZOSIN HYDROCHLORIDE:Schizophrenia:0.26), (TERIFLUNOMIDE:Schizophrenia:0.13), (TESTOST ERONE UNDECANOATE:Schizophrenia:0.29), (TETRACYCLINE:Schizophrenia:0.56), (THALIDOMI DE:Schizophrenia:1.62), (THIAMYLAL SODIUM:Schizophrenia:0.03), (THYROID, PORCINE:Schizophrenia:0.15), (TIPEPIDINE HIBENZATE:Schizophrenia:0.07), (TRAMETINIB:Schizophrenia:0), (TRIENTINE:Schizophren ia:0.39), (UMECLIDINIUM BROMIDEVILANTEROL TRIFENATATE:Schizophrenia:0.91), (VALGANCICLOVIR HYDROCHLORIDE:Schizophrenia:0.94), (ZAFIRLUKAST:Schizophrenia:0.09), (ZOLMITRIP TAN:Schizophrenia:0.5), (ACETAMINOPHENCODEINE PHOSPHATE:Schizophrenia, paranoid type:0.04), (BUPRENORPHINE HYDROCHLORIDE:Schizophrenia, paranoid type:0.69), (BUPRENORPHINE HYDROCHLORIDENALOXONE HYDROCHLORIDE:Schizophrenia, paranoid type:0.16), (DEXLANSOPRAZOLE:Schizophrenia, paranoid type:0.3), (FLUTICASONE PROPIONATE:Schizophrenia, paranoid type:0.5), (IBRUTINIB:Schizophrenia, paranoid type:0.16), (LISDEXAMFETAMINE DIMESYLATE:Schizophrenia, paranoid type:0.15), (MONTELUKAST SODIUM:Schizophrenia, paranoid type:0.14), (PANTOPRAZOLE:Schizophrenia, paranoid type:0.4), (PAROXETINE:Schizophrenia, paranoid type:0.54), (RANITIDINE HYDROCHLORIDE:Schizophrenia, paranoid type:0.33), (VERAPAMIL HYDROCHLORIDE:Schizophrenia, paranoid type:0.39), (WARFARIN:Schizophrenia, paranoid type:0.42), (ZUCLOPENTHIXOL DECANOATE:Schizophrenia, paranoid type:0.17), (.ALPHA.1-PROTEINASE INHIBITOR HUMAN:Sciatica:0.17), (ACETAMINOPHENPROPOXYPHENE HYDROCHLORIDE:Sciatica:0.07), (ACETAZOLAMIDE:Sciatica:0.49), (AGALSIDASE BETA:Sciatica:0.02), (AZILSARTAN KAMEDOXOMIL:Sciatica:0.28), (BENAZEPRILBENAZEPRIL HYDROCHLORIDE:Sciatica:0.11), (BUDESONIDEFORMOTEROL FUMARATE:Sciatica:0.57), (BUTYLSCOPOLAMINE BROMIDE:Sciatica:0.12), (CALCIUM ACETATE:Sciatica:0.68), (CARFILZOMIB:Sciatica:0.6), (CARVEDILOL PHOSPHATE:Sciatica:0.58), (CETIRIZINE HYDROCHLORIDEPSEUDOEPHEDRINE HYDROCHLORIDE:Sciatica:0.02), (CIMETIDINE:Sciatica:0.62), (CLINDAMYCIN:Sciatica:0.18), (CLINDAMYCINCLINDAMYCIN PHOSPHATE:Sciatica:1.01), (CLOZAPINE:Sciatica:0.51), (CORTICOTROPIN:Sciatica:0.2), (D ABIGATRAN:Sciatica:0.3), (DACLIZUMAB:Sciatica:0.67), (DALTEPARIN:Sciatica:0.14), (DA PAGLIFLOZIN PROPANEDIOL:Sciatica:0.35), (DASATINIB:Sciatica:1.28), (DEFLAZACORT:Sciatica:0.33), (DIGITOXIN:Sciatica:0.15), (DIPHENHYDRAMINE:Sciatica:0.89), (DOXEPIN HYDROCHLORIDE:Sciatica:0.27), (DRONABINOL:Sciatica:0.31), (DUTASTERIDETAMSU LOSIN HYDROCHLORIDE:Sciatica:0.03), (ECONAZOLE:Sciatica:0.37), (EMPAGLIFLOZINLINAG LIPTIN:Sciatica:0.39), (ENOXAPARIN:Sciatica:1.4), (EPLERENONE:Sciatica:0.47), (ESTROG ENS, CONJUGATEDMEDROXYPROGESTERONE ACETATE:Sciatica:0.64), (EVEROLIMUS:Sciatica:0.68), (EVOLOCUMAB:Sciatica:1.36), (FE NOFIBRIC ACID:Sciatica:0.08), (FLUDROCORTISONE ACETATE:Sciatica:0.22), (FLUOXETINEFLUOXETINE HYDROCHLORIDE:Sciatica:0.79), (FLUTICASONE:Sciatica:0.56), (FLUTICASONE FUROATE:Sciatica:0.35), (FORMOTEROL FUMARATE:Sciatica:0.51), (FORMOTEROL FUMARATE\MOMETASONE FUROATE:Sciatica:0.11), (FUSIDIC ACID:Sciatica:0.15), (GLYCOPYRRONIUMINDACATEROL:Sciatica:0.63), (GOSERELIN ACETATE:Sciatica:0.01), (GUAIFENESIN:Sciatica:0.25), (HERBALSTURMERIC:Sciatica:0.31), (HYDROXYCHLOROQUINE DIPHOSPHATE:Sciatica:0.28), (HYDROXYZINE PAMOATE:Sciatica:1.11), (HYOSCYAMINE:Sciatica:0.16), (ILOPROST:Sciatica:0.15), (IMAT INIB MESYLATE:Sciatica:0.89), (INFLUENZA VIRUS VACCINE:Sciatica:0.63), (INSULIN GLULISINE:Sciatica:0.12), (LACOSAMIDE:Sciatica:0.27), (LEDIPASVIRSOFOSBUVIR:Scia tica:0.68), (LEVALBUTEROL HYDROCHLORIDE:Sciatica:0.51), (LIOTHYRONINE SODIUM:Sciatica:0.12), (LITHIUM CARBONATE:Sciatica:0.99), (LOMITAPIDE MESYLATE:Sciatica:0.06), (MEGESTROL ACETATE:Sciatica:0.08), (METRONIDAZOLEMETRONIDAZOLE HYDROCHLORIDE:Sciatica:0.78), (MINOCYCLINEMINOCYCLINE HYDROCHLORIDE:Sciatica:0.14), (NATEGLINIDE:Sciatica:0.02), (NICOTINE:Sciatica:0.01), (NORTRIPTYLINE HYDROCHLORIDE:Sciatica:0.16), (OLODATEROL HYDROCHLORIDETIOTROPIUM BROMIDE MONOHYDRATE:Sciatica:0.22), (PANTOPRAZOLE MAGNESIUM:Sciatica:1.3), (PAZOPANIB HYDROCHLORIDE:Sciatica:0.29), (PEMETREXED DISODIUM:Sciatica:0.24), (PENICILLIN:Sciatica:0.88), (PENTOSAN POLYSULFATE SODIUM:Sciatica:0.26), (PROPAFENONE HYDROCHLORIDE:Sciatica:0.09), (PSYLLIUM HUSK:Sciatica:0.12), (PYRIDOXINE:Sciatica:0.06), (RALOXIFENE HYDROCHLORIDE:Sciatica:0.86), (RIZATRIPTAN BENZOATE:Sciatica:0.99), (TERBINAFINE HYDROCHLORIDE:Sciatica:0.2), (TETRACYCLINE:Sciatica:0.64), (THALIDOMIDE:Sciatica: 0.16), (TIMOLOL:Sciatica:0.91), (TIOTROPIUM:Sciatica:0.38), (VITAMIN A:Sciatica:0.15), (ACETAMINOPHENBUTALBITALCAFFEINE:Scleroderma:0.22), (ALBIG LUTIDE:Scleroderma:0.23), (ANASTROZOLE:Scleroderma:0.41), (BISACODYL OR DOCUSATE SODIUM:Scleroderma:0.11), (BUSPIRONE HYDROCHLORIDE:Scleroderma:0.16), (CALCIUM ACETATE:Scleroderma:0.32), (CANDESARTAN CILEXETILHYDROCHLOROTHIAZIDE:Scleroderma:0.12), (CARFILZOMIB:Scleroderma:00.11), (CARVEDILOL PHOSPHATE:Scleroderma:0.15), (CLINDAMYCINCLINDAMYCIN PHOSPHATE:Scleroderma:0.74), (CORTISONEHYDROCORTISONE:Scleroderma:0.04), (DA SATINIB:Scleroderma:0.43), (DEVICE:Scleroderma:0.1), (DIETARY SUPPLEMENT:Scleroderma:0.1), (DOXAZOSINDOXAZOSIN MESYLATE:Scleroderma:0.17), (DUTASTERIDE:Scleroderma:0.02), (ELETRIPTAN HYDROBROMIDE:Scleroderma:0.11), (ESZOPICLONE:Scleroderma:0.59), (EVOLOCUMAB: Scleroderma:0.79), (HYDROCHLOROTHIAZIDELISINOPRIL:Scleroderma:0.39), (HYDROX YUREA:Scleroderma:0.02), (HYDROXYZINE PAMOATE:Scleroderma:0.11), (HYOSCYAMINE:Scleroderma:0.35), (IBRUTINIB:Scleroderm a:1.37), (INSULIN NOS:Scleroderma:0.05), (LABETALOLLABETALOL HYDROCHLORIDE:Scleroderma:0.13), (LEDIPASVIRSOFOSBUVIR:Scleroderma:0.09), (M ETHYLPREDNISOLONE ACETATE:Scleroderma:0), (METRONIDAZOLE:Scleroderma:1.48), (NITROFURANTOIN:Scl eroderma:0.16), (OMEGA-3-ACID ETHYL ESTERS:Scleroderma:0.14), (OXCARBAZEPINE:Scleroderma:0.45), (PANTOPRAZOLE MAGNESIUM:Scleroderma:0.21), (PRIMIDONE:Scleroderma:0.18), (SACUBITRILVALSAR TAN:Scleroderma:0.23), (SECUKINUMAB:Scleroderma:0.38), (SOTALOL:Scleroderma:0.58), (TERIFLUNOMIDE:Scleroderma:0.18), (UMECLIDINIUM BROMIDEVILANTEROL TRIFENATATE:Scleroderma:0.2), (VALGANCICLOVIR HYDROCHLORIDE:Scleroderma:0.2), (VARENICLINE TARTRATE:Scleroderma:0.19), (VITAMIN B:Scleroderma:0.32), (ANASTROZOLE:Scoliosis:0.03), (ARMODAFINIL:Scoliosis:0.43), (BU DESONIDE:Scoliosis:0.34), (BUPROPION:Scoliosis:0.08), (CERTOLIZUMAB PEGOL:Scoliosis:0.59), (CORTISONEHYDROCORTISONE:Scoliosis:0.25), (DEXLANSOPR AZOLE:Scoliosis:1.13), (DIGOXIN:Scoliosis:0.45), (DILTIAZEM HYDROCHLORIDE:Scoliosis:0.54), (ENALAPRIL:Scoliosis:0.65), (ENOXAPARIN:Scoliosis: 0.2), (EVOLOCUMAB:Scoliosis:0.74), (HYDROCHLOROTHIAZIDELISINOPRIL:Scoliosis:00.16), (HYDROXYZINE PAMOATE:Scoliosis:0.16), (IBRUTINIB:Scoliosis:0.83), (ILOPROST:Scoliosis:0.01), (INDOM ETHACIN:Scoliosis:0.39), (LISDEXAMFETAMINE DIMESYLATE:Scoliosis:0.22), (METHYLPREDNISOLONE SODIUM SUCCINATE:Scoliosis:0.24), (METRONIDAZOLE:Scoliosis:0.51), (POLYETHYLENE GLYCOLS:Scoliosis:0.04), (PRIMIDONE:Scoliosis:0.42), (RANOLAZINE:Scoliosis:0.32), (TA MSULOSIN HYDROCHLORIDE:Scoliosis:0.01), (TERIFLUNOMIDE:Scoliosis:0.33), (TOCILIZUMAB:Sc oliosis:0.01), (ABACAVIR SULFATELAMIVUDINE:Seasonal allergy:0.16), (ACETAMINOPHENBUTALBITALCAFFEINECODEINE PHOSPHATE:Seasonal allergy:0.02), (ACETAMINOPHEN\CAFFEINE:Seasonal allergy:0.4), (ACETAMINOPHENPROPOXYPHENE NAPSYLATE:Seasonal allergy:0.62), (AGALSIDASE BETA:Seasonal allergy:1), (ALBIGLUTIDE:Seasonal allergy:0.89), (ALMOTRIPTAN MALATE:Seasonal allergy:0.02), (AMBROXOL:Seasonal allergy:0), (AMILORIDE HYDROCHLORIDEHYDROCHLOROTHIAZIDE:Seasonal allergy:0.95), (AMINO ACIDSDEXTROSEELECTROLYTES NOS:Seasonal allergy:0.5), (AMINO ACIDSDEXTROSEELECTROLYTES NOSSOYBEAN OILVITAMINS:Seasonal allergy:0.44), (AMISULPRIDE:Seasonal allergy:0.81), (AMLODIPINE BESYLATEHYDROCHLOROTHIAZIDEOLMESARTAN MEDOXOMIL:Seasonal allergy:0.39), (AMLODIPINE BESYLATEHYDROCHLOROTHIAZIDEVALSARTAN:Seasonal allergy:0.14), (AMLODIPINE BESYLATETELMISARTAN:Seasonal allergy:0.33), (AMLODIPINEVALSARTAN:Seasonal allergy:0.74), (AMOXAPINE:Seasonal allergy:0.11), (AMOXICILLINCLARITHROMYCINESOMEPRAZOLE:Seasonal allergy:0.07), (ARIPIPRAZOLE LAUROXIL:Seasonal allergy:0.76), (ASCORBIC ACID/CHROMIUM/SELENIUM/VANADYL SULFATE:Seasonal allergy:0.18), (ASPIRIN LYSINE:Seasonal allergy:0.53), (ASPIRIN\CAFFEINESALICYLAMIDE:Seasonal allergy:0.19), (ASPIRINMAGNESIUM OXIDE:Seasonal allergy:0.01), (ASUNAPREVIR:Seasonal allergy:0.35), (ATROPINE SULFATE:Seasonal allergy:0.03), (AVANAFIL:Seasonal allergy:0.24), (AZOSEMIDE:Seasonal allergy:0.39), (BENZOCAINE:Seasonal allergy:0.11), (BENZTROPINE MESYLATE:Seasonal allergy:0.6), (BEPRIDIL:Seasonal allergy:0.1), (BLINATUMOMAB:Seasonal allergy:0.41), (BOTULINUM TOXIN TYPE A:Seasonal allergy:0.47), (BUPRENORPHINENALOXONE:Seasonal allergy:0.08), (BUTYLSCOPOLAMINE BROMIDE:Seasonal allergy:0.66), (CABOZANTINIB S-MALATE:Seasonal allergy:0.26), (CALCITONIN SALMON:Seasonal allergy:1.45), (CALCIUM LACTATE:Seasonal allergy:0.06), (CALCIUM\MAGNESIUM:Seasonal allergy:1.5), (CALCIUM\MAGNESIUM\ZINC:Seasonal allergy:1.42), (CAPSAICIN:Seasonal allergy:1.07), (CAPTOPRIL:Seasonal allergy:1.57), (CARBIMAZOLE:Seasonal allergy:0.12), (CARBOXYMETHYLCELLULOSE SODIUMHYPROMELLOSES:Seasonal allergy:0.03), (CEFAZOLIN:Seasonal allergy:0.54), (CEFOTAXIME SODIUM:Seasonal allergy:0.52), (CEFPODOXIME PROXETIL:Seasonal allergy:0.07), (CEFPROZIL:Seasonal allergy:0.56), (CHLOROTHIAZIDE:Seasonal allergy:0.45), (CICLOPIROX:Seasonal allergy:0.2), (CILASTATIN SODIUMIMIPENEM:Seasonal allergy:0.77), (CIPROFIBRATE:Seasonal allergy:0.37), (CISPLATIN:Seasonal allergy:0.02), (CLIKSTAR:Seasonal allergy:0.06), (CLOMIPRAMINE HYDROCHLORIDE:Seasonal allergy:0.29), (CLOXAZOLAM:Seasonal allergy:0.04), (COBICISTATELVITEGRAVIREMTRICITABINETENOFOVIR ALAFENAMIDE FUMARATE:Seasonal allergy:0.13), (COLESTIPOL:Seasonal allergy:0.63), (COLISTIMETHATE SODIUM:Seasonal allergy:0.2), (CORTICOTROPIN:Seasonal allergy:1.36), (DACLIZUMAB:Seasonal allergy:1.75), (DANTROLENE SODIUM:Seasonal allergy:0.04), (DEGARELIX ACETATE:Seasonal allergy:0.08), (DEXAMETHASONETOBRAMYCIN:Seasonal allergy:0.14), (DIENOGEST:Seasonal allergy:0.33), (DIPYRONE:Seasonal allergy:0.53), (DOCOSANOL:Seasonal allergy:0), (DOLASETRON MESYLATE:Seasonal allergy:0.45), (DOLUTEGRAVIR SODIUM:Seasonal allergy:0.03), (DORZOLAMIDE HYDROCHLORIDE:Seasonal allergy:0.94), (DUTASTERIDETAMSULOSIN HYDROCHLORIDE:Seasonal allergy:1.2), (ELBASVIRGRAZOPREVIR:Seasonal allergy:0.21), (ELOTUZUMAB:Seasonal allergy:0.61), (ELTROMBOPAG OLAMINE:Seasonal allergy:0.76), (EMPAGLIFLOZINLINAGLIPTIN:Seasonal allergy:1.09), (EMTRICITABINERILPIVIRINE HYDROCHLORIDETENOFOVIR DISOPROXIL FUMARATE:Seasonal allergy:0.12), (EPHEDRINE HYDROCHLORIDEGUAIFENESIN:Seasonal allergy:0.49), (ESTRADIOL HEMIHYDRATE:Seasonal allergy:0.44), (ESTRADIOLLEVONORGESTREL:Seasonal allergy:0.51), (ESTRIOL:Seasonal allergy:0.71), (ESTROGENS, ESTERIFIEDMETHYLTESTOSTERONE:Seasonal allergy:0.17), (ETHINYL ESTRADIOLFERROUS FUMARATENORETHINDRONE:Seasonal allergy:0.1), (ETHINYL ESTRADIOLGESTODENE:Seasonal allergy:0.09), (ETORICOXIB:Seasonal allergy:1.43), (ETRETINATE:Seasonal allergy:0.99), (EVOLOCUMAB:Seasonal allergy:2.34), (FELBAMATE:Seasonal allergy:0.3), (FENOTEROL HYDROBROMIDE:Seasonal allergy:0.88), (FENTANYL CITRATE:Seasonal allergy:0.29), (FERROUS BISGLYCINATE:Seasonal allergy:0.15), (FERROUS SULFATE ANHYDROUS:Seasonal allergy:0.43), (FONDAPARINUX SODIUM:Seasonal allergy:0.11), (FOSINOPRIL:Seasonal allergy:0.14), (FOSPHENYTOINFOSPHENYTOIN SODIUM:Seasonal allergy:0.09), (GIMERACILOTERACILTEGAFUR:Seasonal allergy:0.64), (GLIPIZIDEMETFORMIN HYDROCHLORIDE:Seasonal allergy:0.49), (GLYCOPYRROLATEINDACATEROL MALEATE:Seasonal allergy:0.53), (GLYCOPYRRONIUM:Seasonal allergy:1.33), (GUANFACINE:Seasonal allergy:1.45), (HERBALSSAW PALMETTO:Seasonal allergy:0.26), (HYDROCODONE BITARTRATEIBUPROFEN:Seasonal allergy:0.94), (HYDROCORTISONE ACETATE:Seasonal allergy:10.3), (HYDROQUINONE:Seasonal allergy:0.21), (IBRITUMOMAB TIUXETAN:Seasonal allergy:0.05), (IMIGLUCERASE:Seasonal allergy:0.44), (INDAPAMIDEPERINDOPRIL:Seasonal allergy:1.12), (INFLIXIMAB-DYYB:Seasonal allergy:0.4), (INTERFERON:Seasonal allergy:0.92), (INTERFERON ALFA-2A:Seasonal allergy:0.68), (INTERFERON ALFA-2B:Seasonal allergy:0.5), (INTERFERON GAMMA-1B:Seasonal allergy:0.92), (IRINOTECAN:Seasonal allergy:0.44), (IXEKIZUMAB:Seasonal allergy:0.15), (LABETALOL HYDROCHLORIDE:Seasonal allergy:0.78), (LATANOPROSTTIMOLOL:Seasonal allergy:0.49), (LENVATINIB:Seasonal allergy:0.17), (LEVOLEUCOVORIN CALCIUM:Seasonal allergy:0.48), (LUMACAFTOR:Seasonal allergy:0.55), (LUTETIUM LU-177:Seasonal allergy:0.27), (MEGESTROL:Seasonal allergy:0.33), (MEPERIDINE HYDROCHLORIDE:Seasonal allergy:0.37), (MESNA:Seasonal allergy:0.28), (METHENAMINE HIPPURATE:Seasonal allergy:1.66), (METHENAMINE MANDELATE:Seasonal allergy:0.06), (METHENOLONE ENANTHATE:Seasonal allergy:0.07), (MIGLUSTAT:Seasonal allergy:0.15), (MOEXIPRIL HYDROCHLORIDE:Seasonal allergy:0.3), (MOXIFLOXACIN:Seasonal allergy:0.86), (NALOXONE HYDROCHLORIDEOXYCODONE HYDROCHLORIDE:Seasonal allergy:1.32), (NALOXONE HYDROCHLORIDETILIDINE HYDROCHLORIDE:Seasonal allergy:0.09), (NILOTINIB:Seasonal allergy:2.91), (OCTREOTIDE:Seasonal allergy:0.92), (OLMESARTAN:Seasonal allergy:0.16), (OMEPRAZOLE SODIUM:Seasonal allergy:0.49), (OSELTAMIVIR:Seasonal allergy:0.47), (PAMIDRONATE DISODIUM:Seasonal allergy:1.16), (PANITUMUMAB:Seasonal allergy:0.29), (PARICALCITOL:Seasonal allergy:0.34), (PATIROMER:Seasonal allergy:0.52), (PENICILLIN V POTASSIUM:Seasonal allergy:0.2), (PERPHENAZINE:Seasonal allergy:0.02), (POLYETHYLENE GLYCOL 3350POTASSIUM CHLORIDE\SODIUM BICARBONATE\SODIUM CHLORIDE\SODIUM SULFATE ANHYDROUS:Seasonal allergy:0.26), (POLYETHYLENE GLYCOL 400:Seasonal allergy:0.81), (POLYETHYLENE GLYCOL 4000:Seasonal allergy:0), (PONATINIB:Seasonal allergy:1.63), (PREGNENOLONE:Seasonal allergy:0), (PROTRIPTYLINE HYDROCHLORIDE:Seasonal allergy:0.88), (PYRAZINAMIDE:Seasonal allergy:0.78), (QUINACRINE:Seasonal allergy:0.42), (RADIATION THERAPY:Seasonal allergy:0.5), (RAMUCIRUMAB:Seasonal allergy:0.04), (REGORAFENIB:Seasonal allergy:1), (SACUBITRILVALSARTAN:Seasonal allergy:0.79), (SARPOGRELATE:Seasonal allergy:0.59), (SEVELAMER CARBONATE:Seasonal allergy:0.15), (SILICON DIOXIDE:Seasonal allergy:0.07), (SILVER SULFADIAZINE:Seasonal allergy:0.7), (SIPULEUCEL-T:Seasonal allergy:0.35), (STARCH, WHEAT:Seasonal allergy:0.4), (SUPLATAST TOSILATE:Seasonal allergy:0.16), (TASIMELTEON:Seasonal allergy:0.26), (TEDUGLUTIDE\WATER:Seasonal allergy:0.2), (TELOTRISTAT ETHYL:Seasonal allergy:0.21), (TETRACYCLINE:Seasonal allergy:1.76), (THEOPHYLLINE:Seasonal allergy:0.8), (TINZAPARIN SODIUM:Seasonal allergy:0.28), (TIPIRACIL HYDROCHLORIDETRIFLURIDINE:Seasonal allergy:0.26), (TRAMETINIB DIMETHYL SULFOXIDE:Seasonal allergy:0.02), (TREPROSTINIL DIOLAMINE:Seasonal allergy:0.07), (TRIPTORELIN PAMOATE:Seasonal allergy:0.55), (TROSPIUM CHLORIDE:Seasonal allergy:0.83), (UBIQUINOL:Seasonal allergy:0.02), (VALGANCICLOVIR HYDROCHLORIDE:Seasonal allergy:1.11), (VANCOMYCIN HYDROCHLORIDE:Seasonal allergy:0.5), (VINORELBINE TARTRATE:Seasonal allergy:0.4), (VORICONAZOLE:Seasonal allergy:0.82), (WARFARIN POTASSIUM:Seasonal allergy:0.22), (YEAST:Seasonal allergy:0.63), (CERTOLIZUMAB PEGOL:Seborrhoeic dermatitis:0.31), (EVOLOCUMAB:Seborrhoeic dermatitis:0.32), (IBRUTINIB:Seborrhoeic dermatitis:0.09), (METHYLPREDNISOLONE SODIUM SUCCINATE:Seborrhoeic dermatitis:0.09), (OMEPRAZOLE MAGNESIUM:Seborrhoeic dermatitis:0.34), (TERIFLUNOMIDE:Seborrhoeic dermatitis:0.01), (SACUBITRILVALSARTAN:Secondary progressive multiple sclerosis:0.09), (.ALPHA.1-PROTEINASE INHIBITOR HUMAN:Secretion discharge:0.89), (ACETAMINOPHENBUTALBITALCAFFEINE:Secretion discharge:0.01), (ACETAMINOPHENCODEINE PHOSPHATE:Secretion discharge:0.62), (ANASTROZOLE:Secretion discharge:0.02), (CARFILZOMIB:Secretion discharge:0.13), (CHOLESTYRAMINE:Secretion discharge:0.19), (CLINDAMYCINCLINDAMYCIN PHOSPHATE:Secretion discharge:0.64), (COLCHICINE:Secretion discharge:0.24), (DICYCLOMINE HYDROCHLORIDE:Secretion discharge:0.05), (ELETRIPTAN HYDROBROMIDE:Secretion discharge:0.03), (ENALAPRIL:Secretion discharge:0.99), (ENOXAPARIN:Secretion discharge:0.59), (EVOLOCUMAB:Secretion discharge:0.43), (EZETIMIBE:Secretion discharge:0.12), (FENOFIBRATE:Secretion discharge:0.3), (FINGOLIMOD HYDROCHLORIDE:Secretion discharge:0.2), (FLECAINIDE:Secretion discharge:0.24), (FORMOTEROL FUMARATE\MOMETASONE FUROATE:Secretion discharge:1), (IBRUTINIB:Secretion discharge:0.58), (ILOPROST:Secretion discharge:0.41), (INFLUENZA VIRUS VACCINE:Secretion discharge:0.08), (IRBESARTAN:Secretion discharge:0.04), (ISOSORBIDE MONONITRATE:Secretion discharge:0.14), (LISDEXAMFETAMINE DIMESYLATE:Secretion discharge:0.04), (LOVASTATIN:Secretion discharge:0.17), (LUMACAFTOR:Secretion discharge:0.51), (MEPOLIZUMAB:Secretion discharge:0.79), (METAXALONE:Secretion discharge:0.15), (MODAFINIL:Secretion discharge:0.02), (MOXIFLOXACIN HYDROCHLORIDE:Secretion discharge:0.1), (NEBIVOLOL:Secretion discharge:0.37), (PYRAZINAMIDE:Secretion discharge:0.61), (RABEPRAZOLE SODIUM:Secretion discharge:0.13), (RANOLAZINE:Secretion discharge:0.45), (SECUKINUMAB:Secretion discharge:0.11), (SOTALOL:Secretion discharge:0), (TELMISARTAN:Secretion discharge:0.12), (TOCILIZUMAB:Secretion discharge:0.31), (TOFACITINIB CITRATE:Secretion discharge:0.05), (TRAMADOL HYDROCHLORIDE:Secretion discharge:0.31), (ZOLPIDEM TARTRATE:Secretion discharge:0.27), (ABOBOTULINUMTOXINA:Sedation:0.08), (ACENOCOUMAROL:Sedation: 1.03), (ACETAMINOPHENOXYCODONEOXYCODONE HYDROCHLORIDE:Sedation:0.99), (ACETAMINOPHENPROPOXYPHENE NAPSYLATE:Sedation:0.26), (ACETIC ACIDSODIUM ACETATE:Sedation:0.15), (ACLIDINIUM BROMIDE:Sedation:0.24), (AGALSIDASE BETA:Sedation:0.4), (ALCOHOL:Sedation:1.64), (ALEMTUZUMAB:Sedation:0.82), (ALFUZO SIN:Sedation:0.11), (ALFUZOSIN HYDROCHLORIDE:Sedation:0.33), (ALISKIREN:Sedation:0.12), (ALUMINUM HYDROXIDEMAGNESIUM OXIDEOXETHAZAINE:Sedation:0.19), (AMLODIPINEVALSARTAN:Sedation:0.7), (AMM ONIUM GLYCYRRHIZATECYSTEINE HYDROCHLORIDEGLYCINE:Sedation:0.24), (AMOXAPINE:Sedation:0.02), (ANAGRELID E:Sedation:0.06), (ANASTROZOLE:Sedation:1.45), (ASENAPINE MALEATE:Sedation:0.05), (AZILSARTAN KAMEDOXOMIL:Sedation:0.68), (BECLOMETHASONE:Sedation:0.07), (BECLOMETHASO NEFORMOTEROL:Sedation:0.18), (BENAZEPRILBENAZEPRIL HYDROCHLORIDE:Sedation:0.46), (BENZTROPINE MESYLATE:Sedation:0.6), (BETAMIPRONPANIPENEM:Sedation:0.45), (BILASTINE:Sedati on:0.37), (BISACODYLGLYCERINMINERAL OILSODIUM PHOSPHATE, DIBASIC\SODIUM PHOSPHATE, MONOBASIC:Sedation:0.09), (BLESELUMAB:Sedation:0.57), (BLINATUMOMAB:Sedation:10.08), (BOTULISM ANTITOXIN:Sedation:0.37), (BRINZOLAMIDE:Sedation:0.29), (BROMPERIDOL:Sedation:0.23), (BUPRENORPHINE HYDROCHLORIDENALOXONE HYDROCHLORIDE:Sedation:0.7), (CALCIUM CARBONATEVITAMIN D:Sedation:0.52), (CANAKINUMAB:Sedation:0.48), (CARBIDOPA:Sedation:0), (CARBON DIOXIDE:Sedation:0.36), (CARVEDILOL PHOSPHATE:Sedation:0.8), (CEFAMANDOLE:Sedation:0.2), (CEFPIROME SULFATE:Sedation:0.19), (CEFPODOXIME PROXETIL:Sedation:0.22), (CETUXIMAB:Sedation:0.46), (CICLESONIDE:Sedation:0.5), (CLE MASTINE FUMARATE:Sedation:0.13), (CLENBUTEROL HYDROCHLORIDE:Sedation:0.22), (CLOFARABINE:Sedation:0.19), (COD LIVER OIL:Sedation:0.06), (CODEINE PHOSPHATEGUAIFENESIN:Sedation:0), (COLISTIMETHATE SODIUM:Sedation:0.6), (CORTISONEHYDROCORTISONE:Sedation:1.28), (CROTALIDAE POLYVALENT IMMUNE FAB (OVINE):Sedation:0.17), (DACLIZUMAB:Sedation:1.06), (DANAZOL:Sedation:0.63), (DAPAG LIFLOZIN PROPANEDIOL:Sedation:0.14), (DAPSONE:Sedation:0.66), (DEFLAZACORT:Sedation:0.53), (DEXLANSOPRAZOLE:Sedation:1.32), (DEXTROAMPHETAMINE SULFATE:Sedation:0.01), (DEXTROSE MONOHYDRATE\SODIUM CHLORIDE:Sedation:0.06), (DIAMORPHINE:Sedation:0.12), (DICYCLOMINE HYDROCHLORIDE:Sedation:0.55), (DIMETHICONESILICON DIOXIDESIMETHICONE:Sedation:0.32), (DIMETHINDENE:Sedation:0.1), (DOFETILIDE:Se dation:0.38), (DOXEPIN HYDROCHLORIDE:Sedation:0.12), (DRONEDARONE:Sedation:0.01), (EDOXABAN TOSYLATE:Sedation:1.32), (EMPAGLIFLOZINLINAGLIPTIN:Sedation:0.34), (ENTACAPO NE:Sedation:0.21), (ETORICOXIB:Sedation:0.88), (EVOLOCUMAB:Sedation:0.77), (FENOFIB RIC ACID:Sedation:0.02), (FERROUS GLUCONATE:Sedation:0.51), (FERROUS SULFATE ANHYDROUS:Sedation:0.14), (FERROUS SULFATEFOLIC ACID:Sedation:0.04), (FERROUS SULFATEGLYCINE:Sedation:1.26), (FESOTERODINE FUMARATE:Sedation:0.95), (FLUDROCORTISONE ACETATE:Sedation:0.85), (FLUVASTATIN SODIUM:Sedation:0.29), (FONDAPARINUX:Sedation:0.64), (FORMOTEROL FUMARATE DIHYDRATE\MOMETASONE FUROATE:Sedation:0.55), (FORMOTEROL FUMARATE\MOMETASONE FUROATE:Sedation:0.24), (GIMERACILOTERACILTEGAFUR:Sedation:0.12), (GLYCOPY RROLATEINDACATEROL MALEATE:Sedation:0.03), (GUSPERIMUS TRIHYDROCHLORIDE:Sedation:0.38), (HALOPERIDOL DECANOATE:Sedation:0.26), (HYDROCHLOROTHIAZIDELISINOPRIL:Sedation:1.43), (H YDROCHLOROTHIAZIDETELMISARTAN:Sedation:0.87), (HYDROCHLOROTHIAZIDE VALSARTAN:Sedation:1.09), (IBRITUMOMAB TIUXETAN:Sedation:0.6), (IDARUCIZUMAB:Sedation:0.46), (ILOPROST:Sedation:0.72), (IMI PRAMINE HYDROCHLORIDE:Sedation:0.51), (INDAPAMIDEPERINDOPRIL:Sedation:1.2), (INTERFE RON:Sedation:0.17), (INTERFERON ALFA-2A:Sedation:0.01), (IOPROMIDE:Sedation:0.07), (IRINOTECAN HYDROCHLORIDE:Sedation:0.33), (ISOSORBIDE:Sedation:0.89), (LACTOBACILLUS ACIDOPHILUS\LACTOBACILLUS DELBRUECKII SUBSP. BULGARICUS:Sedation:0.06), (LACTOBACILLUS RHAMNOSUS:Sedation:0.22), (LERCANIDIPINE HYDROCHLORIDE:Sedation:1.13), (LEVAMLODIPINE BESYLATE:Sedation:0.27), (LEVOLEUCOVORIN CALCIUM:Sedation:0.5), (LOVASTATIN:Sedation:0.82), (MAGNESIUM ASPARTATE HYDROCHLORIDE:Sedation:0.35), (MAGNESIUM ASPARTATE\POTASSIUM ASPARTATE:Sedation:0), (MAGNESIUM CHLORIDE\POTASSIUM CHLORIDE\SODIUM ACETATE\SODIUM CHLORIDE\SODIUM GLUCONATE:Sedation:0.07), (MECLIZINE HYDROCHLORIDE:Sedation:0.14), (MECLIZINEMECLIZINE HYDROCHLORIDE:Sedation:0.05), (MEMANTINE HYDROCHLORIDE:Sedation:0.51), (MEPOLIZUMAB:Sedation:0.04), (METARAMINOL:Sed ation:0.66), (METAXALONE:Sedation:0.75), (METHENAMINE HIPPURATE:Sedation:1.22), (METHENOLONE ENANTHATE:Sedation:0.95), (METHOCARBAMOL:Sedation:0.84), (METHYLNALTREXO NE:Sedation:0.06), (METHYLPREDNISOLONE OR METHYLPREDNISOLONE ACETATE:Sedation:0.15), (METILDIGOXIN:Sedation:0.69), (MIGLUSTAT:Sedation:0.57), (M ILRINONE LACTATE:Sedation:0.42), (MITOXANTRONE HYDROCHLORIDE:Sedation:0.16), (MOGAMULIZUMAB:Sedation:0.07), (MORPHINE SULFATE\NALTREXONE HYDROCHLORIDE:Sedation:0.02), (MORPHINE\MORPHINE HYDROCHLORIDE\MORPHINE SULFATE\MORPHINE TARTRATE:Sedation:1.07), (MOXONIDINE:Sedation:0.77), (NADOLOL:Sedation:0.24), (NAT EGLINIDE:Sedation:0.19), (NIACIN:Sedation:0.56), (NINTEDANIB:Sedation:0.11), (NIRAPAR 1B:Sedation:0.05), (NORETHINDRONE:Sedation:0.06), (OXYMORPHONE:Sedation:0.31), (PA ZUFLOXACIN MESILATE:Sedation:0.24), (PEGASPARGASE:Sedation:0.74), (PEGINTERFERON ALFA-2B:Sedation:0.08), (PEGINTERFERON BETA-1A:Sedation:0.29), (PEMETREXED:Sedation:0.69), (PEMETREXED DISODIUM:Sedation:0.45), (PENTAMIDINE ISETHIONATE:Sedation:0.54), (PENTOSTATIN:Sedation:0.16), (PERAMPANEL:Sedation:0.13), (PERPHENAZINE:Sedation:0.09), (PHENELZINE:Sedation:0.18), (PILSICAINIDE HYDROCHLORIDE:Sedation:0.02), (PIRENZEPINE:Sedation:0.45), (PITAVASTATIN CALCIUM:Sedation:1.46), (PONATINIB:Sedation:0.56), (PRAZEPAM:Sedation:0.46), (PREDN ISOLONE SODIUM PHOSPHATE:Sedation:1.36), (PROCATEROL HYDROCHLORIDE:Sedation:0.34), (RALOXIFENE HYDROCHLORIDE:Sedation:0.03), (RANOLAZINE:Sedation:1.2), (ROFLUMILAST:Sedation: 1.03), (RUSCUS ACULEATUS ROOT:Sedation:0.16), (SALMETEROL XINAFOATE:Sedation:0.11), (SECUKINUMAB:Sedation:1.18), (SELEXIPAG:Sedation:0.22), (SENNOSIDE B:Sedation:0.08), (SEVELAMER HYDROCHLORIDE:Sedation:0.17), (SODIUM CITRATE:Sedation:1.37), (SODIUM FERROUS CITRATE:Sedation:0.78), (SODIUM FLUORIDE:Sedation:0.17), (SOTALOL HYDROCHLORIDE:Sedation:0.18), (SUCCINYLATED GELATIN:Sedation:0.29), (SUGAMMADEX SODIUM:Sedation:1.42), (SUPLATAST TOSILATE:Sedation:0.11), (TABALUMAB:Sedation:0.16), (TAFAMIDIS MEGLUMINE:Sedation:0.13), (TERIFLUNOMIDE:Sedation:0.02), (TESTOSTERONE UNDECANOATE:Sedation:0.04), (THROMBOPOIETIN:Sedation:0.65), (TIAPRIDE:Sedation: 0.2), (TISAGENLECLEUCEL:Sedation:0.04), (TRIHEXYPHENIDYL:Sedation:1.2), (TRIMIPR AMINE MALEATE:Sedation:0.62), (TROSPIUM CHLORIDE:Sedation:0.54), (VALGANCICLOVIR HYDROCHLORIDE:Sedation:1.93), (VILAZODONE HYDROCHLORIDE:Sedation:0.23), (VOGLIBOSE:Sedation:0.36), (ZICONOTIDE ACETATE:Sedation:0.62), (ZINC:Sedation:0.29), (ZUCLOPENTHIXOL DECANOATE:Sedation:0.59), (ACARBOSE:Sedative therapy:0.25), (ACETAMINOPHENPROPOXYPHENE HYDROCHLORIDE:Sedative therapy:0.08), (ACETAMINOPHENTRAMADOL:Sedative therapy:0.02), (AGALSIDASE BETA:Sedative therapy:0.05), (ALEMTUZUMAB:Sedative therapy:0.12), (ALENDRONIC ACID:Sedative therapy:0.35), (AMINO ACIDSDEXTROSEELECTROLYTES NOS:Sedative therapy:0), (BENAZEPRILBENAZEPRIL HYDROCHLORIDE:Sedative therapy:0.06), (BENZTROPINE MESYLATE:Sedative therapy:0.05), (BISACODYL OR DOCUSATE SODIUM:Sedative therapy:0.9), (CALCIUMVITAMIN D:Sedative therapy:0.68), (CANDESARTAN:Sedative therapy:1.3), (CARFILZOMIB:Sedative therapy:0.54), (CARVEDILOL PHOSPHATE:Sedative therapy:0.53), (CEFUROXIME:Sedative therapy:0.65), (CEPHALEXIN:Sedative therapy:1.46), (CIMETIDINE:Sedative therapy:0.22), (CISATRACURIUM BESYLATE:Sedative therapy:0.26), (CLOMIPRAMINE HYDROCHLORIDE:Sedative therapy:0.54), (CYPROHEPTADINE:Sedative therapy:0.17), (DACLIZUMAB:Sedative therapy:0.2), (DALFAMPRIDINE:Sedative therapy:0.26), (DALTEPARIN:Sedative therapy:0.2), (DANAZOL:Sedative therapy:0.25), (DAPAGLIFLOZIN PROPANEDIOL:Sedative therapy:0.07), (DESVENLAFAXINE SUCCINATE:Sedative therapy:0.41), (DEXAMETHASONE ACETATE:Sedative therapy:0.09), (DOXEPIN HYDROCHLORIDE:Sedative therapy:0.07), (ECONAZOLE:Sedative therapy:0.17), (EDOXABAN TOSYLATE:Sedative therapy:0.45), (ELETRIPTAN HYDROBROMIDE:Sedative therapy:0.65), (EPLERENONE:Sedative therapy:0.36), (ESMOLOL HYDROCHLORIDE:Sedative therapy:1.13), (EVEROLIMUS:Sedative therapy:0.7), (EVOLOCUMAB:Sedative therapy:0.82), (FEBUXOSTAT:Sedative therapy:0.67), (FERROUS GLUCONATE:Sedative therapy:0.13), (FERROUS SULFATEGLYCINE:Sedative therapy:0.09), (FESOTERODINE FUMARATE:Sedative therapy:0.42), (FLUDROCORTISONE ACETATE:Sedative therapy:0.12), (FLUTICASONE FUROATEVILANTEROL TRIFENATATE:Sedative therapy:0.07), (FLUVOXAMINE MALEATE:Sedative therapy:0.13), (FORMOTEROL:Sedative therapy:0.14), (FORMOTEROL FUMARATE:Sedative therapy:0.18), (FORMOTEROL FUMARATE DIHYDRATE\MOMETASONE FUROATE:Sedative therapy:0.09), (GUANFACINE HYDROCHLORIDE:Sedative therapy:0.18), (HEPARIN CALCIUM:Sedative therapy:0.02), (HYDROXYCHLOROQUINE DIPHOSPHATE:Sedative therapy:0.29), (HYOSCYAMINE:Sedative therapy:0.05), (IDELALISIB:Sedative therapy:0.02), (IMATINIB MESYLATE:Sedative therapy:0.23), (IMIPRAMINE:Sedative therapy:0.04), (IMIPRAMINE HYDROCHLORIDE:Sedative therapy:0.05), (IMMUNE GLOBULIN NOS:Sedative therapy:0.17), (INDAPAMIDEPERINDOPRIL:Sedative therapy:0.69), (LABETALOL HYDROCHLORIDE:Sedative therapy:0.22), (LERCANIDIPINE:Sedative therapy:1.34), (LORMETAZEPAM:Sedative therapy:0), (METAXALONE:Sedative therapy:0.54), (METHENAMINE HIPPURATE:Sedative therapy:0.34), (METOLAZONE:Sedative therapy:0.27), (MIDODRINE:Sedative therapy:0.44), (MODAFINIL:Sedative therapy:0.22), (MOMETASONE FUROATE:Sedative therapy:0.1), (MORPHINE\MORPHINE HYDROCHLORIDE\MORPHINE SULFATE\MORPHINE TARTRATE:Sedative therapy:0.09), (NADOLOL:Sedative therapy:0.17), (NITROFURANTOIN:Sedative therapy:0.85), (NORTRIPTYLINE:Sedative therapy:0.64), (OMEGA-3-ACID ETHYL ESTERS:Sedative therapy:0.6), (PANTOPRAZOLE MAGNESIUM:Sedative therapy:0.75), (PAZOPANIB HYDROCHLORIDE:Sedative therapy:0.18), (PENTOBARBITAL:Sedative therapy:0.37), (PITAVASTATIN CALCIUM:Sedative therapy:0.31), (PONATINIB:Sedative therapy:0.21), (PREDNISOLONE SODIUM PHOSPHATE:Sedative therapy:0.31), (PROBIOTICS NOS:Sedative therapy:1.13), (PROGESTERONE:Sedative therapy:0.04), (RALOXIFENE HYDROCHLORIDE:Sedative therapy:0.27), (RANOLAZINE:Sedative therapy:1.35), (ROPINIROLE HYDROCHLORIDE:Sedative therapy:0.7), (SACUBITRILVALSARTAN:Sedative therapy:0.33), (SODIUM CITRATE:Sedative therapy:0.12), (SUGAMMADEX SODIUM:Sedative therapy:0.28), (SUMATRIPTANSUMATRIPTAN SUCCINATE:Sedative therapy:0.54), (TAZOBACTAM:Sedative therapy:0.08), (TERBINAFINE HYDROCHLORIDE:Sedative therapy:0.03), (TERBUTALINE:Sedative therapy:0.64), (TETRACYCLINE:Sedative therapy:0.05), (THALIDOMIDE:Sedative therapy:1.08), (TIMOLOL:Sedative therapy:0.37), (TOLTERODINE TARTRATE:Sedative therapy:0.24), (TRIAMCINOLONE ACETONIDE:Sedative therapy:0.52), (TRIHEXYPHENIDYL:Sedative therapy:0.24), (ZINC:Sedative therapy:0.02), (.ALPHA.1-PROTEINASE INHIBITOR HUMAN:Seizure:1.54), (ABACAVIR:Seizure:0.45), (ACITRETIN:Seizure:0.43), (ACTIVATED CHARCOAL:Seizure:1.11), (ALANYL GLUTAMINE:Seizure:0.28), (ALISKIREN HEMIFUMARATE:Seizure:0.95), (AMINOSALICYLIC ACID:Seizure:0.42), (AMPHETAMINE SULFATE:Seizure:0.43), (AMPHETAMINEDEXTROAMPHETAMINE:Seizure:0.1), (ANAG RELIDE:Seizure:0.24), (ANAGRELIDE HYDROCHLORIDE:Seizure:0.07), (ANIDULAFUNGIN:Seizure:0.17), (ANTIHEMOPHILIC FACTOR, HUMAN RECOMBINANT:Seizure:0.19), (ANTIHISTAMINES NOS:Seizure:0.92), (ASPERGILLUS NIGER .ALPHA.-GALACTOSIDASE:Seizure:0), (ASPIRIN\CAFFEINESALICYLAMIDE:Seizure:0.02), (ASPI RINMAGNESIUM OXIDE:Seizure:0.62), (ASUNAPREVIR:Seizure:0.44), (ATENOLOLCHLORTHALIDONE:Sei zure:0.46), (ATRACURIUM:Seizure:0.69), (ATRACURIUM BESYLATE:Seizure:0.38), (AZELNIDIPINE:Seizure:0.02), (BALSALAZIDE DISODIUM:Seizure:0.35), (BECLOMETHASONEFORMOTEROL:Seizure:0.49), (BENAZEP RIL HYDROCHLORIDEHYDROCHLOROTHIAZIDE:Seizure:0), (BENDAMUSTINE HYDROCHLORIDE:Seizure:1), (BERAPROST:Seizure:0.14), (BETAMETHASONECALCIPO TRIENE:Seizure:0.58), (BILASTINE:Seizure:0.08), (BIPERIDEN:Seizure:0.42), (BLINATUMO MAB:Seizure:1.97), (BLONANSERIN:Seizure:0.3), (BRENTUXIMAB VEDOTIN:Seizure:0.3), (BUDESONIDEFORMOTEROL:Seizure:0.53), (BUPIVACAINE HYDROCHLORIDE:Seizure:0.81), (BUPRENORPHINENALOXONE:Seizure:0.76), (CABAZI TAXEL:Seizure:0.3), (CALCIPOTRIENE:Seizure:0.5), (CALCIUM CARBONATEERGOCALCIFEROL:Seizure:0.53), (CALCIUM CHLORIDEDEXTROSE MONOHYDRATEMAGNESIUM CHLORIDE\SODIUM CHLORIDE\SODIUM LACTATE:Seizure:0.16), (CALCIUM PANTOTHENATECYANOCOBALAMINNIACINAMIDEPYRIDOXINE HYDROCHLORIDE\RIBOFLAVINTHIAMINE MONONITRATE:Seizure:0.07), (CALCIUM\MAGNESIUM\ZINC:Seizure:1.39), (CALCIUM VITAMINS:Seizure:0.07), (CANAKINUMAB:Seizure:1.07), (CANDESARTAN CILEXETILHYDROCHLOROTHIAZIDE:Seizure:2.4), (CAPSAICIN:Seizure:0.77), (CARBID OPA:Seizure:0.77), (CARBOHYDRATESFATTY ACIDSMINERALSPROTEINVITAMINS:Seizure:0.12), (CARVEDILOL PHOSPHATE:Seizure:2.81), (CASPOFUNGIN:Seizure:1.68), (CEFOXITINCEFOXITIN SODIUM:Seizure:0), (CEFUROXIME SODIUM:Seizure:0.03), (CHLOROTHIAZIDE:Seizure:1.58), (CHLORPROTHIXENE:Seizure:00.46), (CODEINE PHOSPHATEGUAIFENESIN:Seizure:1.14), (COLLAGEN:Seizure:0.02), (CYAMEMAZINE:S eizure:0.59), (CYSTEAMINE BITARTRATE:Seizure:0.68), (DACARBAZINE:Seizure:0.03), (DAPAGLIFLOZIN PROPANEDIOLMETFORMIN HYDROCHLORIDE:Seizure:0.26), (DARUNAVIR:Seizure:1.14), (DEFERIPRONE:Seizure:0.28), (DEGARELIX:Seizure:0.09), (DIETARY SUPPLEMENTRESVERATROL:Seizure:0), (DIFLUNISAL:Seizure:0.24), (DIMETHICONES ILICON DIOXIDESIMETHICONE:Seizure:1.25), (DIMETHINDENE:Seizure:0.03), (DINUTUXIMAB: Seizure:0.01), (DIPYRONEPROMETHAZINE HYDROCHLORIDE:Seizure:0.66), (DOLASETRON MESYLATE:Seizure:0.8), (DROPERIDOL:Seizure:0.17), (DUTASTERIDETAMSULOSIN HYDROCHLORIDE:Seizure:1.11), (ELOSULFASE ALFA:Seizure:0.2), (EMPAGLIFLOZINLINAGLIPTIN:Seizure:1.21), (EMTRICITABINERIL PIVIRINE HYDROCHLORIDETENOFOVIR DISOPROXIL FUMARATE:Seizure:0.18), (EMTRICITABINERILPIVIRINETENOFOVIR DISOPROXIL FUMARATE:Seizure:0.11), (ENALAPRIL MALEATELERCANIDIPINE HYDROCHLORIDE:Seizure:0.38), (ENFUVIRTIDE:Seizure:0.03), (EPHEDRINE HYDROCHLORIDEGUAIFENESIN:Seizure:0.15), (EPINASTINE HYDROCHLORIDE:Seizure:0.67), (ERIBULIN:Seizure:0.67), (ESMOLOL HYDROCHLORIDE:Seizure:1.13), (ESTRADIOL BENZOATE:Seizure:0.09), (ESTROGENS:Seizure:1.11), (ETHINYL ESTRADIOLNORGESTREL:Seizure:0.81), (ETIMICIN SULFATE:Seizure:0.76), (ETOPOSIDE PHOSPHATE:Seizure:0.24), (ETORICOXIB:Seizure:1.11), (ETRETINATE:Seizure:0.22), (EVO LOCUMAB:Seizure:2.22), (FENOPROFEN CALCIUM:Seizure:0.01), (FENOTEROL HYDROBROMIDE:Seizure:0.59), (FENOTEROL HYDROBROMIDEIPRATROPIUM BROMIDE:Seizure:0.52), (FERRIC CARBOXYMALTOSE:Seizure:0.05), (FERROUS BISGLYCINATE:Seizure:0.23), (FERROUS SULFATEGLYCINE:Seizure:0.6), (FLUOCINOLONE ACETONIDE:Seizure:0.83), (FLUOROMETHOLONE:Seizure:0.21), (FLUPENTIXOL:Seizure: 0.19), (FORMOTEROL:Seizure:1.36), (FORMOTEROL FUMARATE\MOMETASONE FUROATE:Seizure:1.33), (FUSIDIC ACID:Seizure:1.19), (GIMERACILOTERACILTEGAFUR:Seizure:0.93), (GINGER:Seizure:0.56), (GLIPIZIDEMETFORMIN HYDROCHLORIDE:Seizure:0.21), (GLYCOPYRROLATEINDACATEROL MALEATE:Seizure:0.75), (GUSPERIMUS TRIHYDROCHLORIDE:Seizure:0.1), (HALOPERIDOL DECANOATE:Seizure:0.84), (HUMAN PLASMA:Seizure:0.15), (HYALURONATE SODIUM:Seizure:0.88), (HYDROCHLOROTHIAZIDESPIRONOLACTONE:Seizure:0.41), (H YOSCYAMINE SULFATEMETHENAMINEMETHYLENE BLUEPHENYL SALICYLATE\SODIUM PHOSPHATE, MONOBASIC, MONOHYDRATE:Seizure:0.22), (IBRITUMOMAB TIUXETAN:Seizure:0.79), (IDARUCIZUMAB:Seizure:0.47), (INSULIN PORKINSULIN PURIFIED PORK:Seizure:0.06), (INTERFERON:Seizure:1.66), (INTERFERON ALFA-2A:Seizure:1.45), (INTERFERON ALFA-2B:Seizure:1.79), (IRONPOLYSACCHARIDES:Seizure:0.25), (IVACAFTORLUMACAFTOR: Seizure:0.47), (LAFUTIDINE:Seizure:0.06), (LANREOTIDE:Seizure:0.42), (LENVATINIB:Sei zure:0.59), (LEVAMLODIPINE BESYLATE:Seizure:0.16), (LEVOBUPIVACAINE:Seizure:0.24), (LEVOLEUCOVORIN CALCIUM:Seizure:0.9), (LORMETAZEPAM:Seizure:0.58), (LOXOPROFEN:Seizure:0.01), (LU TETIUM LU-177:Seizure:0.1), (MEPOLIZUMAB:Seizure:1.1), (METFORMIN HYDROCHLORIDESAXAGLIPTIN:Seizure:0.07), (METFORMIN HYDROCHLORIDEVILDAGLIPTIN:Seizure:0.83), (METHENAMINE MANDELATE:Seizure:0.17), (METHOXY POLYETHYLENE GLYCOL-EPOETIN BETA:Seizure:0.06), (METHYLCELLULOSE (4000 MPA. S):Seizure:0.15), (METRELEPTIN:Seizure:0.27), (MIZORIBINE:Seizure:0.25), (NADROP ARIN CALCIUM:Seizure:1.65), (NALOXONE HYDROCHLORIDE:Seizure:0.5), (NALOXONE HYDROCHLORIDETILIDINE HYDROCHLORIDE:Seizure:0.5), (NEFAZODONE:Seizure:0.16), (NEOSTIGMINE:Seizure:1.44), (NINTEDANIB:Seizure:1.26), (NUSINERSEN:Seizure:1.52), (OXYBUTYNINOXYBUTYN IN CHLORIDE:Seizure:0.43), (PANITUMUMAB:Seizure:0.58), (PARATHYROID HORMONE:Seizure:0.47), (PEGINTERFERON ALFA-2B:Seizure:0.73), (PENTAMIDINE ISETHIONATE:Seizure:0.66), (PENTOSTATIN:Seizure:0.83), (PHENAZOPYRIDINE HYDROCHLORIDE:Seizure:0.28), (PHENELZINE:Seizure:0.63), (PILOCARPINE:Seizure:0.9), (PILSICAINIDE HYDROCHLORIDE:Seizure:0.14), (PIMOBENDAN:Seizure:0.07), (PNEUMOCOCCAL VACCINE, POLYVALENT 23:Seizure:0.82), (POLYETHYLENE GLYCOL 3350POTASSIUM CHLORIDE\SODIUM BICARBONATE\SODIUM CHLORIDE\SODIUM SULFATE ANHYDROUS:Seizure:0.35), (PRAMLINTIDE ACETATE:Seizure:0.11), (PRAZEPAM:Seizure:0.46), (PREDNISOLONE SODIUM PHOSPHATE:Seizure:1.64), (PREGNENOLONE:Seizure:0.07), (PRISTINAMYCIN:Seizure:0.18), (PROMAZINE HYDROCHLORIDE:Seizure:0.07), (PROPARACAINE:Seizure:0.2), (PYRIDOSTIGMINE:Seiz ure:0.13), (QUINACRINE:Seizure:0.14), (RADIUM RA-223 DICHLORIDE:Seizure:0.12), (REGORAFENIB:Seizure:1.72), (RETINOL:Seizure:1.58), (RILM ENIDINE PHOSPHATE:Seizure:0.3), (RILPIVIRINE HYDROCHLORIDE:Seizure:0.37), (ROFECOXIB:Seizure:0.23), (ROPIVACAINE:Seizure:0.31), (RUCAPARIB CAMSYLATE:Seizure:0.25), (SEBELIPASE ALFA:Seizure:0.09), (SELEXIPAG:Seizure:0.56), (SIBUTRAMINE HYDROCHLORIDE:Seizure:0.18), (SODIUM CITRATE:Seizure:0.81), (SODIUM FERROUS CITRATE:Seizure:0.01), (SODIUM PHOSPHATE, DIBASIC\SODIUM PHOSPHATE, MONOBASIC:Seizure:1.24), (SOMATREM:Seizure:0.11), (SOTALOL HYDROCHLORIDE:Seizure:0.58), (SUFENTANIL CITRATE:Seizure:1.32), (SULBACTAM SODIUM:Seizure:0.07), (TAFLUPROST:Seizure:0.24), (TAMOXIFEN CITRATE:Seizure:0.31), (TBO-FILGRASTIM:Seizure:0.1), (TEDUGLUTIDE\WATER:Seizure:1.22), (TELOTRISTAT ETHYL:Seizure:0.55), (TERBUTALINE SULFATE:Seizure:2.3), (TESAMORELIN:Seizure:0.07), (TESTOSTERONE UNDECANOATE:Seizure:0.73), (TETRACYCLINE:Seizure:1.64), (THEOPHYLLINE:Seizure: 0.75), (THIOGUANINE:Seizure:0.19), (TICLOPIDINE:Seizure:0.16), (TRANYLCYPROMINE: Seizure:0.07), (TRANYLCYPROMINE SULFATE:Seizure:0.02), (TRETINOIN:Seizure:0.82), (TRICHLORMETHIAZIDE:Seizure:0.53), (TRIMIPRAMINE MALEATE:Seizure:0.74), (UROKINASE:Seizure:0.31), (VALDECOXIB:Seizure:0.22), (VAND ETANIB:Seizure:0.01), (VARDENAFIL HYDROCHLORIDE TRIHYDRATE:Seizure:0.78), (VINORELBINEVINORELBINE TARTRATE:Seizure:0.69), (YEAST:Seizure:0.56), (ZILEUTON:Seizure:0.77), (ZOTEPINE:Seiz ure:0.2), (ZUCLOPENTHIXOL DECANOATE:Seizure:0.67), (AMIODARONE:Selective IgG subclass deficiency:0.21), (ANASTROZOLE:Selective IgG subclass deficiency:0.29), (BISACODYL OR DOCUSATE SODIUM:Selective IgG subclass deficiency:0.5), (BISOPROLOL FUMARATE:Selective IgG subclass deficiency:0.11), (BUDESONIDEFORMOTEROL FUMARATE:Selective IgG subclass deficiency:0.08), (CALCIUM CARBONATEVITAMIN D:Selective IgG subclass deficiency:0.24), (CALCIUM CITRATE:Selective IgG subclass deficiency:0.61), (CERTOLIZUMAB PEGOL:Selective IgG subclass deficiency:1.17), (CLINDAMYCIN:Selective IgG subclass deficiency:0.01), (DESLORATADINE:Selective IgG subclass deficiency:0.07), (DIGOXIN:Selective IgG subclass deficiency:0.67), (DIMENHYDRINATE:Selective IgG subclass deficiency:0.22), (ENALAPRIL:Selective IgG subclass deficiency:0.97), (ENALAPRIL MALEATE:Selective IgG subclass deficiency:0.07), (ESOMEPRAZOLE MAGNESIUMNAPROXEN:Selective IgG subclass deficiency:0.13), (EVOLOCUMAB:Selective IgG subclass deficiency:0.5), (EZETIMIBESIMVASTATIN:Selective IgG subclass deficiency:0.06), (FLUOXETINEFLUOXETINE HYDROCHLORIDE:Selective IgG subclass deficiency:0.03), (FLUTICASONE FUROATEVILANTEROL TRIFENATATE:Selective IgG subclass deficiency:0.42), (FORMOTEROL FUMARATE\MOMETASONE FUROATE:Selective IgG subclass deficiency:0.25), (ILOPROST:Selective IgG subclass deficiency:0.02), (IMIPRAMINE HYDROCHLORIDE:Selective IgG subclass deficiency:0.03), (INDOMETHACIN:Selective IgG subclass deficiency:0.33), (LACTASE:Selective IgG subclass deficiency:0.05), (LOVASTATIN:Selective IgG subclass deficiency:0.79), (MEPOLIZUMAB:Selective IgG subclass deficiency:0.18), (METHYLPREDNISOLONE ACETATE:Selective IgG subclass deficiency:0.56), (MOXIFLOXACIN HYDROCHLORIDE:Selective IgG subclass deficiency:0.09), (NORTRIPTYLINE:Selective IgG subclass deficiency:0.58), (OLANZAPINE:Selective IgG subclass deficiency:0.13), (OMEGA-3-ACID ETHYL ESTERS:Selective IgG subclass deficiency:0.05), (OXCARBAZEPINE:Selective IgG subclass deficiency:0.98), (PANTOPRAZOLE MAGNESIUM:Selective IgG subclass deficiency:0.85), (PAROXETINEPAROXETINE HYDROCHLORIDE:Selective IgG subclass deficiency:0.06), (PENICILLIN:Selective IgG subclass deficiency:0.47), (PHENOBARBITAL:Selective IgG subclass deficiency:0.04), (PRAVASTATIN SODIUM:Selective IgG subclass deficiency:0.47), (PRIMIDONE:Selective IgG subclass deficiency:0.37), (PYRIDOXINE:Selective IgG subclass deficiency:0.15), (SODIUM OXYBATE:Selective IgG subclass deficiency:1.37), (SULFAMETHOXAZOLE:Selective IgG subclass deficiency:0.04), (SUMATRIPTAN:Selective IgG subclass deficiency:0.01), (TERBUTALINE SULFATE:Selective IgG subclass deficiency:0.08), (TERIFLUNOMIDE:Selective IgG subclass deficiency:0.05), (TETRACYCLINE:Selective IgG subclass deficiency:0.09), (TOFACITINIB CITRATE:Selective IgG subclass deficiency:0.34), (TOLTERODINE TARTRATE:Selective IgG subclass deficiency:0.18), (UMECLIDINIUM BROMIDEVILANTEROL TRIFENATATE:Selective IgG subclass deficiency:0.66), (VARENICLINE TARTRATE:Selective IgG subclass deficiency:1.23), (WARFARIN:Selective IgG subclass deficiency:0.9), (ENALAPRIL:Senile osteoporosis:0.21), (POTASSIUM:Senile osteoporosis:0.1), (ACETAMINOPHENBUTALBITALCAFFEINE:Sepsis:0.51), (ACETAMIN OPHENTRAMADOL:Sepsis:0.09), (ACLARUBICIN HYDROCHLORIDE:Sepsis:0.77), (ACLIDINIUM BROMIDE:Sepsis:0.21), (ADO-TRASTUZUMAB EMTANSINE:Sepsis:0.11), (AGALSIDASE BETA:Sepsis:0.16), (ALCOHOL:Sepsis:0.3), (ALEMTUZUMAB:Sepsis:0.17), (ALUMINUM HYDROXIDEMAGNESIUM OXIDEOXETHAZAINE:Sepsis:0.03), (AMITRIPTYLINE HYDROCHLORIDE:Sepsis:0.32), (AMLODIPINEPERINDOPRIL:Sepsis:0.18), (AMLODIPIN EVALSARTAN:Sepsis:0.07), (AMMONIUM GLYCYRRHIZATECYSTEINE HYDROCHLORIDEGLYCINE:Sepsis:0.16), (AMPHETAMINE ASPARTATEAMPHETAMINE SULFATEDEXTROAMPHETAMINE SACCHARATEDEXTROAMPHETAMINE SULFATE:Sepsis:0.17), (ANASTROZOLE:Sepsis:0.74), (ATRACURIUM BESYLATE:Sepsis:0.58), (ATROPINE SULFATE:Sepsis:0.27), (BELATACEPT:Sepsis:0.14), (BENDAMUSTINE HYDROCHLORIDE:Sepsis:0.54), (BETAMETHASONE SODIUM PHOSPHATE:Sepsis:0.3), (BISACODYL OR DOCUSATE SODIUM:Sepsis:0.93), (BLESELUMAB:Sepsis:0.04), (BLINATUMOMAB:Sepsis:1.92), (BUDE SONIDEFORMOTEROL:Sepsis:0.07), (CALCITONIN SALMON:Sepsis:0.3), (CALCIUM CITRATE:Sepsis:0.25), (CALCIUMVITAMIN D:Sepsis:0.6), (CANAGLIFLOZINMETFORMIN HYDROCHLORIDE:Sepsis:0.09), (CANAKINUMAB:Sepsis:0.47), (CANDESARTAN CILEXETILHYDROCHLOROTHIAZIDE:Sepsis:1.08), (CARVEDILOL PHOSPHATE:Sepsis:0.48), (CEFPODOXIME PROXETIL:Sepsis:0.04), (CEFUROXIME AXETIL:Sepsis:0.07), (CETUXIMAB:Sepsis:0.51), (CHLORTHALIDONE:Sepsis:0.21), (CISAT RACURIUM:Sepsis:0.56), (CISATRACURIUM BESYLATE:Sepsis:0.02), (CLADRIBINE:Sepsis:0.09), (CLOBAZAM:Sepsis:0.65), (CORTISO NEHYDROCORTISONE:Sepsis:0.72), (CYCLOPHOSPHAMIDECYTARABINEDEXAMET HASONEDOXORUBICINMETHOTREXATEVINCRISTINE:Sepsis:0.24), (CYPROHEPTA DINE:Sepsis:0.08), (DABIGATRAN:Sepsis:0.43), (DACLIZUMAB:Sepsis:0.6), (DANAZOL:Se psis:0.7), (DAPAGLIFLOZIN PROPANEDIOL:Sepsis:0.07), (DEFLAZACORT:Sepsis:0.23), (DESFLURANE:Sepsis:0.83), (D EXAMETHASONE ACETATE:Sepsis:0.95), (DEXLANSOPRAZOLE:Sepsis:1.05), (DIGITOXIN:Sepsis:0.2), (DIM ETHICONESILICON DIOXIDESIMETHICONE:Sepsis:0.18), (DOCUSATE CALCIUM:Sepsis:0.16), (DOFETILIDE:Sepsis:0.07), (DONEPEZIL:Sepsis:0.04), (DOXEPIN:Se psis:0.03), (ECONAZOLE:Sepsis:1.01), (EDOXABAN TOSYLATE:Sepsis:0.86), (ELETRIPTAN HYDROBROMIDE:Sepsis:0.16), (EPINASTINE HYDROCHLORIDE:Sepsis:0.21), (ESMOLOL HYDROCHLORIDE:Sepsis:0.57), (ETOPOSIDE PHOSPHATE:Sepsis:0.3), (EVOLOCUMAB:Sepsis:0.28), (EZETIMIBESIMVASTATIN:Sepsi s:0.11), (FESOTERODINE FUMARATE:Sepsis:0.39), (FLECAINIDE:Sepsis:1.14), (FLUTICASONE FUROATE:Sepsis:0.02), (FLUTICASONE FUROATEVILANTEROL TRIFENATATE:Sepsis:0.94), (FORMOTEROL FUMARATE DIHYDRATE\MOMETASONE FUROATE:Sepsis:0.15), (FOSPHENYTOIN SODIUM:Sepsis:0.31), (FUSIDIC ACID:Sepsis:0.61), (GARENOXACIN:Sepsis:0.36), (GIMERACILOTERACILTEGAFUR:Sep sis:0.31), (GLYCOPYRRONIUMINDACATEROL:Sepsis:0.67), (HYDROCHLOROTHIAZIDE IRBESARTAN:Sepsis:0.57), (HYDROCHLOROTHIAZIDELOSARTAN POTASSIUM:Sepsis:0.2), (HYDROCHLOROTHIAZIDETELMISARTAN:Sepsis:0.24), (HYO SCYAMINE:Sepsis:0.14), (IBRITUMOMAB TIUXETAN:Sepsis:1.14), (ILOPROST:Sepsis:0.92), (INDAPAMIDEPERINDOPRIL:Sepsis:1.15), (INSULIN GLULISINE:Sepsis:0.23), (INTERFERON ALFA-2A:Sepsis:0.15), (INTERFERON GAMMA-1B:Sepsis:0.36), (IRINOTECAN HYDROCHLORIDE:Sepsis:0.81), (IXAZOMIB:Sepsis:0.21), (LEDIPASVIRSOFOSBUVIR:Se psis:0.53), (LEVALBUTEROL:Sepsis:0.09), (LEVALBUTEROL HYDROCHLORIDE:Sepsis:0.98), (LISDEXAMFETAMINE DIMESYLATE:Sepsis:0.22), (LOVASTATIN:Sepsis:0.86), (MAGNESIUM ASPARTATE HYDROCHLORIDE:Sepsis:0.1), (METAXALONE:Sepsis:0.26), (METHADONE HYDROCHLORIDE:Sepsis:0.94), (METHENAMINE HIPPURATE:Sepsis:0.37), (METHENOLONE ENANTHATE:Sepsis:0.82), (METHIMAZOLE:Sepsis:0.21), (MOGAMULIZUMAB:Sepsis:0.27), (MOXONIDINE:Sepsis:0.17), (NADOLOL:Sepsis:0.08), (NALOXONE HYDROCHLORIDETILIDINE HYDROCHLORIDE:Sepsis:0.4), (NEBIVOLOL HYDROCHLORIDE:Sepsis:0.58), (NIACIN:Sepsis:0.11), (NORTRIPTYLINE:Sepsis:0.36), (OL ODATEROL HYDROCHLORIDETIOTROPIUM BROMIDE MONOHYDRATE:Sepsis:0.33), (OLOPATADINE:Sepsis:0.04), (OMEGA-3-ACID ETHYL ESTERS:Sepsis:0.44), (PANCRELIPASE:Sepsis:0.4), (PANITUMUMAB:Sepsis:0.07), (PANTO PRAZOLE MAGNESIUM:Sepsis:0.85), (PAROXETINEPAROXETINE HYDROCHLORIDE:Sepsis:0.68), (PAZOPANIB HYDROCHLORIDE:Sepsis:0.4), (PEMETREXED:Sepsis:0.46), (PENTOSTATIN:Sepsis:1.17), (PRAMIPEXOLE DIHYDROCHLORIDE:Sepsis:0.72), (PREDNISOLONE SODIUM PHOSPHATE:Sepsis:0.91), (PRIMIDONE:Sepsis:0.87), (PYRIMETHAMINE:Sepsis:0), (RAMU CIRUMAB:Sepsis:0.28), (RETINOL:Sepsis:0.27), (RIZATRIPTAN BENZOATE:Sepsis:0.01), (ROFLUMILAST:Sepsis:0.58), (ROPINIROLE HYDROCHLORIDE:Sepsis:0.39), (ROPIVACAINE:Sepsis:0.09), (ROPIVACAINE HYDROCHLORIDE:Sepsis:0.03), (SACUBITRILVALSARTAN:Sepsis:0.16), (SALMETERO L:Sepsis:0.07), (SEVELAMER HYDROCHLORIDE:Sepsis:0.01), (SODIUM CITRATE:Sepsis:0.2), (SODIUM FERROUS CITRATE:Sepsis:0.07), (SOLIFENACIN SUCCINATE:Sepsis:0.2), (SUCCINYLCHOLINE CHLORIDE:Sepsis:0.54), (SUMATRIPTAN:Sepsis:0.12), (SUMATRIPTANSUMATRIPTAN SUCCINATE:Sepsis:0.13), (TEMOZOLOMIDE:Sepsis:0.04), (TENIPOSIDE:Sepsis:0.02), (TER BINAFINE HYDROCHLORIDE:Sepsis:0.26), (TERBUTALINE:Sepsis:0.82), (TERIFLUNOMIDE:Sepsis:00.01), (THIAMYLAL SODIUM:Sepsis:0.41), (TIMOLOL:Sepsis:0.63), (TISAGENLECLEUCEL:Sepsis:0.42), (TOLTE RODINE TARTRATE:Sepsis:0.05), (TOPIRAMATE:Sepsis:2.1), (TROPATEPINE:Sepsis:0.03), (VEMUR AFENIB:Sepsis:0.31), (VINDESINE SULFATE:Sepsis:0.2), (VITAMIN A:Sepsis:0.04), (WARFARIN POTASSIUM:Sepsis:0.11), (CEFOTAXIMECEFOTAXIME SODIUM:Septic shock:0.01), (DIGOXIN:Septic shock:0.53), (DILTIAZEM:Septic shock:0.06), (ENALAPRIL:Septic shock:0.5), (FLUTICASONE PROPIONATE:Septic shock:0.13), (IBRITUMOMAB TIUXETAN:Septic shock:0.06), (IBRUTINIB:Septic shock:0.68), (IDELALISIB:Septic shock:0.06), (MOXIFLOXACIN HYDROCHLORIDE:Septic shock:0.04), (NILOTINIB:Septic shock:0.06), (SULFAMETHOXAZOLE:Septic shock:0.04), (TAMSULOSIN:Septic shock:0.01), (THALIDOMIDE:Septic shock:0.54), (VALGANCICLOVIR HYDROCHLORIDE:Septic shock:0.98), (PYRAZINAMIDE:Serous cystadenocarcinoma ovary:0.55), (BRIVARACETAM:Severe myoclonic epilepsy of infancy:0.08), (BUDESONIDE:Sexual dysfunction:0.12), (BUMETANIDE:Sexual dysfunction:0.08), (CALCIUM CARBONATECHOLECALCIFEROL:Sexual dysfunction:0.16), (DEVICE:Sexual dysfunction:0.06), (DILTIAZEM:Sexual dysfunction:0.65), (DIVALPROEX SODIUM:Sexual dysfunction:0.52), (EVOLOCUMAB:Sexual dysfunction:0.49), (IBRUTINIB:Sexual dysfunction:0.31), (LANSOPRAZOLE:Sexual dysfunction:0.21), (LORATADINE:Sexual dysfunction:0.1), (OXCARBAZEPINE:Sexual dysfunction:0.13), (OXYGEN:Sexual dysfunction:0.04), (TERIFLUNOMIDE:Sexual dysfunction:0.62), (TORSEMIDE:Sexual dysfunction:0.01), (VARENICLINE TARTRATE:Sexual dysfunction:0.09), (AMOXICILLIN:Short-bowel syndrome:0.02), (ARMODAFINIL:Short-bowel syndrome:0.12), (DICLOFENAC:Short-bowel syndrome:0.17), (DILTIAZEM HYDROCHLORIDE:Short-bowel syndrome:0.59), (DOCUSATE SODIUM:Short-bowel syndrome:0.67), (DULOXETINE:Short-bowel syndrome:0.35), (ESZOPICLONE:Short-bowel syndrome:0.36), (FERROUS SULFATE:Short-bowel syndrome:0.66), (IBRUTINIB:Short-bowel syndrome:0.67), (ILOPROST:Short-bowel syndrome:0.09), (INDOMETHACIN:Short-bowel syndrome:0.24), (LISDEXAMFETAMINE DIMESYLATE:Short-bowel syndrome:0.1), (LOVASTATIN:Short-bowel syndrome:0.4), (METHYLPREDNISOLONE SODIUM SUCCINATE:Short-bowel syndrome:0.12), (NAPROXEN:Short-bowel syndrome:0.87), (NAPROXEN SODIUM:Short-bowel syndrome:0.14), (PANTOPRAZOLE MAGNESIUM:Short-bowel syndrome:0.15), (POLYETHYLENE GLYCOLS:Short-bowel syndrome:0.12), (PRAMIPEXOLE DIHYDROCHLORIDE:Short-bowel syndrome:0.22), (PRAVASTATIN:Short-bowel syndrome:0.38), (ROSUVASTATIN CALCIUM:Short-bowel syndrome:0.28), (SODIUM OXYBATE:Short-bowel syndrome:0.39), (SOTALOL:Short-bowel syndrome:0.01), (TERIFLUNOMIDE:Short-bowel syndrome:0), (TOCILIZUMAB:Short-bowel syndrome:0.18), (TRIAMCINOLONE ACETONIDE:Short-bowel syndrome:0.06), (ATORVASTATIN CALCIUM:Sickle cell anaemia:0.27), (BISOPROLOL:Sickle cell anaemia:0.09), (BISOPROLOL FUMARATE:Sickle cell anaemia:0), (CEPHALEXIN:Sickle cell anaemia:0.36), (CERTOLIZUMAB PEGOL:Sickle cell anaemia:0.3), (DILTIAZEM HYDROCHLORIDE:Sickle cell anaemia:0.68), (ESCITALOPRAM OXALATE:Sickle cell anaemia:0.4), (EVOLOCUMAB:Sickle cell anaemia:0), (IBRUTINIB:Sickle cell anaemia:0.23), (LENALIDOMIDE:Sickle cell anaemia:0.44), (METHYLPREDNISOLONE SODIUM SUCCINATE:Sickle cell anaemia:0.29), (PANTOPRAZOLE:Sickle cell anaemia:0.4), (PRAVASTATIN SODIUM:Sickle cell anaemia:0.23), (SECUKINUMAB:Sickle cell anaemia:0.13), (SELEXIPAG:Sickle cell anaemia:0.49), (TERIFLUNOMIDE:Sickle cell anaemia:0.14), (VENLAFAXINE HYDROCHLORIDE:Sickle cell anaemia:0.17), (PYRAZINAMIDE:Silicotuberculosis:0.52), (.ALPHA.1-PROTEINASE INHIBITOR HUMAN:Sinus congestion:0.08), (ACETAMINOPHENBUTALBITALCAFFEINE:Sinus congestion:0.72), (BISOPROLOL FUMARATE:Sinus congestion:0.15), (BUDESONIDEFORMOTEROL FUMARATE:Sinus congestion:0.06), (CANDESARTAN:Sinus congestion:0.02), (CARFILZOMIB:Sinus congestion:0.22), (CARVEDILOL PHOSPHATE:Sinus congestion:0.24), (CERTOLIZUMAB PEGOL:Sinus congestion:0.56), (CHOLESTYRAMINE:Sinus congestion:0.51), (CLONIDINE:Sinus congestion:0.41), (COLCHICINE:Sinus congestion:0.46), (CORTISONEHYDROCORTISONE:Sinus congestion:0.29), (DASATINIB:Sinus congestion:0.27), (DICLOFENAC:Sinus congestion:0.43), (DICYCLOMINE HYDROCHLORIDE:Sinus congestion:0.59), (DIVALPROEX SODIUM:Sinus congestion:0.77), (DOXAZOSIN MESYLATE:Sinus congestion:0.05), (ENOXAPARIN:Sinus congestion:0.63), (ESTROGENS, CONJUGATEDMEDROXYPROGESTERONE ACETATE:Sinus congestion:0.05), (EVOLOCUMAB:Sinus congestion:0.49), (FESOTERODINE FUMARATE:Sinus congestion:0.22), (FLECAINIDE:Sinus congestion:0.28), (FORMOTEROL FUMARATE\MOMETASONE FUROATE:Sinus congestion:0.02), (HYDROXYZINE PAMOATE:Sinus congestion:0.78), (HYOSCYAMINE:Sinus congestion:0.18), (IMIPRAMINE HYDROCHLORIDE:Sinus congestion:0.11), (INFLUENZA VIRUS VACCINE:Sinus congestion:0.05), (INSULIN HUMAN:Sinus congestion:0.05), (ISOSORBIDE:Sinus congestion:0.39), (LISDEXAMFETAMINE DIMESYLATE:Sinus congestion:0.6), (METAXALONE:Sinus congestion:0.96), (METHYLPREDNISOLONE ACETATE:Sinus congestion:0.07), (METRONIDAZOLE:Sinus congestion:1.34), (METRONIDAZOLEMETRONIDAZOLE HYDROCHLORIDE:Sinus congestion:0.27), (MORPHINE:Sinus congestion:0.58), (NILOTINIB:Sinus congestion:0.44), (NORTRIPTYLINE:Sinus congestion:0.52), (NYSTATIN:Sinus congestion:0.34), (OXYCODONE HYDROCHLORIDE:Sinus congestion:0.23), (PANTOPRAZOLE MAGNESIUM:Sinus congestion:0.56), (PRAMIPEXOLE:Sinus congestion:0.03), (PROGESTERONE:Sinus congestion:0.09), (RALOXIFENE HYDROCHLORIDE:Sinus congestion:0.16), (RISEDRONATE SODIUM:Sinus congestion:0.24), (RIZATRIPTAN BENZOATE:Sinus congestion:0.53), (SACUBITRILVALSARTAN:Sinus congestion:0.81), (SECUKINUMAB:Sinus congestion:0.1), (TETRACYCLINE:Sinus congestion:0.03), (THALIDOMIDE:Sinus congestion:0.08), (TIMOLOL:Sinus congestion:0.31), (TIZANIDINE HYDROCHLORIDE:Sinus congestion:0.98), (TRIAMTERENE:Sinus congestion:0.28), (UMECLIDINIUM BROMIDEVILANTEROL TRIFENATATE:Sinus congestion:0.8), (ZINC:Sinus congestion:0.06), (ZOPICLONE:Sinus congestion:0.04), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Sinus disorder:0.15), (.ALPHA.1-PROTEINASE INHIBITOR HUMAN:Sinus disorder:1.17), (ACARBOSE:Sinus disorder:0.08), (ACETAMINOPHENPROPOXYPHENE NAPSYLATE:Sinus disorder:0.09), (ACETAZOLAMIDE:Sinus disorder:1), (ALBIGLUTIDE:Sinus disorder:0.61), (ALISKIREN HEMIFUMARATE:Sinus disorder:0.18), (ALUMINUM HYDROXIDEDIMETHICONEMAGNESIUM HYDROXIDE:Sinus disorder:0.18), (AMANTADINE:Sinus disorder:0.42), (AMLODIPINE BESYLATEOLMESARTAN MEDOXOMIL:Sinus disorder:0.11), (LONGUM SUBSP. INFANTIS:Sinus disorder:0.75), (BRINZOLAMIDE:Sinus disorder:0.75), (BUDESONIDEFORMOTEROL FUMARATE:Sinus disorder:0.53), (BUPRENORPHINE HYDROCHLORIDENALOXONE HYDROCHLORIDE:Sinus disorder:1.2), (BUTYLSCOPOLAMINE BROMIDE:Sinus disorder:0.37), (CALCIUM POLYCARBOPHIL:Sinus disorder:0.06), (CALCIUM\MAGNESIUM:Sinus disorder:0.35), (CANAGLIFLOZINMETFORMIN HYDROCHLORIDE:Sinus disorder:0.63), (CANAKINUMAB:Sinus disorder:0.25), (CANDESARTAN:Sinus disorder:1.09), (CANDESARTAN CILEXETILHYDROCHLOROTHIAZIDE:Sinus disorder:0.73), (CEFTRIAXONE SODIUM:Sinus disorder:0.2), (CHLORDIAZEPOXIDE HYDROCHLORIDECLIDINIUM BROMIDE:Sinus disorder:0.46), (CLOZAPINE:Sinus disorder:0.33), (CODEINE:Sinus disorder:0.5), (CODEINE PHOSPHATEGUAIFENESIN:Sinus disorder:0.04), (DACLIZUMAB:Sinus disorder:0.93), (DARBEPOETIN ALFA:Sinus disorder:1), (DIMENHYDRINATE:Sinus disorder:0.61), (DRONABINOL:Sinus disorder:0.65), (EDOXABAN TOSYLATE:Sinus disorder:0.31), (EMPAGLIFLOZINLINAGLIPTIN:Sinus disorder:0.38), (EPLERENONE:Sinus disorder:0.08), (EVOLOCUMAB:Sinus disorder:1.76), (FLECAINIDE:Sinus disorder:1.05), (FLUDROCORTISONE ACETATE:Sinus disorder:0.09), (FORMOTEROL FUMARATE:Sinus disorder:0.77), (GLYCOPYRROLATE:Sinus disorder:0.15), (GLYCOPYRRONIUM:Sinus disorder:0.35), (GLYCOPYRRONIUMINDACATEROL:Sinus disorder:0.49), (GUANFACINE:Sinus disorder:0.38), (HYDROCHLOROTHIAZIDETELMISARTAN:Sinus disorder:0.14), (HYDROCODONE BITARTRATEIBUPROFEN:Sinus disorder:0.38), (HYOSCYAMINE SULFATE:Sinus disorder:0.18), (ILOPROST:Sinus disorder:0.5), (IMATINIB MESYLATE:Sinus disorder:0.37), (IMIPRAMINE HYDROCHLORIDE:Sinus disorder:0.75), (INSULIN DEGLUDEC:Sinus disorder:0.06), (LERCANIDIPINE:Sinus disorder:0.08), (LITHIUM:Sinus disorder:0.27), (LOMITAPIDE MESYLATE:Sinus disorder:0.56), (MAGNESIUM GLUCONATE:Sinus disorder:0.08), (MEGESTROL ACETATE:Sinus disorder:0.22), (METHENAMINE HIPPURATE:Sinus disorder:0.9), (MINOCYCLINEMINOCYCLINE HYDROCHLORIDE:Sinus disorder:0.77), (NALOXONE HYDROCHLORIDEOXYCODONE HYDROCHLORIDE:Sinus disorder:0.37), (NALTREXONE:Sinus disorder:0.25), (NORETHINDRONE:Sinus disorder:0.61), (OXCARBAZEPINE:Sinus disorder:1.57), (PANTOPRAZOLE MAGNESIUM:Sinus disorder:1.95), (PATIROMER:Sinus disorder:0.23), (PAZOPANIB HYDROCHLORIDE:Sinus disorder:0.45), (PENTOSAN POLYSULFATE SODIUM:Sinus disorder:0.36), (PERINDOPRIL:Sinus disorder:0.03), (PHENOBARBITAL:Sinus disorder:0.39), (PHYTONADIONE:Sinus disorder:0.09), (PIMECROLIMUS:Sinus disorder:0.52), (PIRFENIDONE:Sinus disorder:0.04), (PIROXICAM:Sinus disorder:0.22), (PONATINIB:Sinus disorder:0.94), (QUETIAPINE:Sinus disorder:1.01), (RALOXIFENE HYDROCHLORIDE:Sinus disorder:0.94), (RETINOL:Sinus disorder:0.05), (ROSUVASTATIN:Sinus disorder:0.16), (SECUKINUMAB:Sinus disorder:0.64), (SENNOSIDES:Sinus disorder:0.21), (SOLIFENACIN SUCCINATE:Sinus disorder:0.61), (SULFAMETHOXAZOLE:Sinus disorder:0.67), (TERBUTALINE SULFATE:Sinus disorder:0.99), (TETRACYCLINE:Sinus disorder:1.18), (THALIDOMIDE:Sinus disorder:0.3), (TIMOLOL MALEATE:Sinus disorder:0.13), (VANCOMYCIN:Sinus disorder:0.04), (ZILEUTON:Sinus disorder:0.19), (ZOPICLONE:Sinus disorder:0.6), (AMPHETAMINE ASPARTATEAMPHETAMINE SULFATEDEXTROAMPHETAMINE SACCHARATEDEXTROAMPHETAMINE SULFATE:Sinus headache:0.08), (DEXLANSOPRAZOLE:Sinus headache:0.33), (DILTIAZEM HYDROCHLORIDE:Sinus headache:0.33), (DIPHENHYDRAMINE:Sinus headache:0.24), (EPINEPHRINE:Sinus headache:0.13), (FERROUS SULFATEFERROUS SULFATE, DRIED:Sinus headache:0.01), (IBRUTINIB:Sinus headache:0.34), (IRON:Sinus headache:0.16), (METRONIDAZOLE:Sinus headache:0.39), (POTASSIUM:Sinus headache:0.17), (TERIFLUNOMIDE:Sinus headache:0.45), (TOCILIZUMAB:Sinus headache:0.04), (NAPROXEN:Sinus operation:0.15), (ANASTROZOLE:Sinus tachycardia:0.09), (BUMETANIDE:Sinus tachycardia:0.1), (CANDESARTAN CILEXETIL:Sinus tachycardia:0.05), (DALFAMPRIDINE:Sinus tachycardia:0.04), (ENALAPRIL:Sinus tachycardia:0.73), (ESOMEPRAZOLE:Sinus tachycardia:0.01), (EVOLOCUMAB:Sinus tachycardia:0.56), (IBRUTINIB:Sinus tachycardia:1.07), (ILOPROST:Sinus tachycardia:0.04), (NILOTINIB:Sinus tachycardia:0.19), (OXYCODONE:Sinus tachycardia:0.02), (OXYGEN:Sinus tachycardia:0.18), (PANTOPRAZOLE:Sinus tachycardia:0.45), (PRAMIPEXOLE DIHYDROCHLORIDE:Sinus tachycardia:0.24), (PRAVASTATIN:Sinus tachycardia:0.19), (TELMISARTAN:Sinus tachycardia:0.03), (TERIFLUNOMIDE:Sinus tachycardia:0.48), (TOCILIZUMAB:Sinus tachycardia:0.02), (TOFACITINIB CITRATE:Sinus tachycardia:0.22), (VARENICLINE TARTRATE:Sinus tachycardia:0.35), (ACENOCOUMAROL:Sinusitis:0.42), (ACETAMINOPHEN\CAFFEINE:Sin usitis:0.05), (ACETAMINOPHENPROPOXYPHENE NAPSYLATE:Sinusitis:0.83), (ACITRETIN:Sinusitis:0.4), (AGALSIDASE BETA:Sinusitis:0.79), (ALBIGLUTIDE:Sinusitis:0.34), (ALBUTEROLIPRATROPIUM BROMIDE:Sinusitis:0), (AMIKACIN SULFATE:Sinusitis:0.02), (AMILORIDE HYDROCHLORIDEHYDROCHLOROTHIAZIDE:Sinusitis:0.68), (AMISULPRIDE:Sinusitis: 0.63), (AMLODIPINE BESYLATEOLMESARTAN MEDOXOMIL:Sinusitis:0.25), (AMLODIPINEVALSARTAN:Sinusitis:0.69), (AMMONIUM LACTATE:Sinusitis:0.91), (ANAKINRA:Sinusitis:O.1), (ANTIHISTAMINES NOS:Sinusitis:0.85), (ARIPIPRAZOLE LAUROXIL:Sinusitis:0.21), (ATENOLOLCHLORTHALIDONE:Sinusitis:0), (ATROPINE SULFATE:Sinusitis:0.08), (AZILSARTAN KAMEDOXOMIL:Sinusitis:0.29), (BELATACEPT:Sinusitis:0.26), (BETAMETHASONE DIPROPIONATECALCIPOTRIENE:Sinusitis:0.03), (BETAMETHASONECALCIPOTRIENE: Sinusitis:0.9), (BOTULINUM TOXIN TYPE A:Sinusitis:0.25), (BUPRENORPHINE HYDROCHLORIDE:Sinusitis:1.38), (CABOZANTINIB S-MALATE:Sinusitis:0.06), (CALCIUM ACETATE:Sinusitis:1.33), (CALCIUM CARBONATEERGOCALCIFEROLRETINOL:Sinusitis:0.52), (CALCIUM\MAGNESIUM\ZINC: Sinusitis:0.73), (CARVEDILOL PHOSPHATE:Sinusitis:1.9), (CASANTHRANOLDOCUSATE SODIUM:Sinusitis:0.36), (CETUXIMAB:Sinusitis:0), (COLISTIMETHATE SODIUM:Sinusitis:0.77), (CORTISONE ACETATE:Sinusitis:0.16), (DACLIZUMAB:Sinusitis:2.09), (DANAZOL:Sinusitis:0.94), (DANT ROLENE SODIUM:Sinusitis:0.3), (DEXAMETHASONE ACETATE:Sinusitis:0.46), (DEXTROAMPHETAMINE:Sinusitis:0.27), (DIFLUNISAL:Sinusitis: 0.05), (DIGITOXIN:Sinusitis:0.02), (DIPYRIDAMOLE:Sinusitis:0.65), (DOCETAXEL:Sinusitis: 0.86), (DOLASETRON MESYLATE:Sinusitis:0.3), (DRONABINOL:Sinusitis:1.35), (ECONAZOLE:Sinusitis:1.03), (ED OXABAN TOSYLATE:Sinusitis:0.94), (ELOTUZUMAB:Sinusitis:0.74), (ELTROMBOPAG OLAMINE:Sinusitis:0.32), (EMTRICITABINERILPIVIRINE HYDROCHLORIDETENOFOVIR DISOPROXIL FUMARATE:Sinusitis:0.14), (ERYTHROPOIETIN:Sinusitis:1.44), (ETHINYL ESTRADIOLNORGESTREL:Sinusitis:0.82), (ETRETINATE:Sinusitis:0.24), (FERROUS SULFATE ANHYDROUS:Sinusitis:0.28), (FLUCYTOSINE:Sinusitis:0.11), (FLUOCINOLONE ACETONIDE:Sinusitis:1.25), (FLUOXETINE HYDROCHLORIDEOLANZAPINE:Sinusitis:0.02), (FLURAZEPAMFLURAZEPAM HYDROCHLORIDE:Sinusitis:0.16), (GIMERACILOTERACILTEGAFUR:Sinusitis:0.15), (GL IPIZIDEMETFORMIN HYDROCHLORIDE:Sinusitis:0.11), (GLYCERINHYPROMELLOSESPOLYETHYLENE GLYCOL 400:Sinusitis:0.07), (GLYCOPYRROLATEINDACATEROL MALEATE:Sinusitis:0.57), (GUANFACINE:Sinusitis:0.53), (GUANFACINE HYDROCHLORIDE:Sinusitis:0.4), (HYDROCODONE BITARTRATEIBUPROFEN:Sinusitis:0.75), (HYOSCYAMINE SULFATEMETHENAMINEMETHYLENE BLUEPHENYL SALICYLATE\SODIUM PHOSPHATE, MONOBASIC, MONOHYDRATE:Sinusitis:0.08), (ILOPROST:Sinusitis:0.84), (IMIPENEM:Sinusitis:0.69), (IM IPRAMINE HYDROCHLORIDE:Sinusitis:1.81), (INDACATEROL:Sinusitis:1.02), (INFLIXIMAB-DYYB:Sinusitis:0.62), (INSULIN DEGLUDEC:Sinusitis:0.59), (INTERFERON:Sinusitis:0.52), (INTERFERON GAMMA-1B:Sinusitis:1.44), (ITRACONAZOLE\SODIUM CHLORIDE:Sinusitis:0.46), (LABETALOL HYDROCHLORIDE:Sinusitis:1.06), (LAPATINIB DITOSYLATE:Sinusitis:0.24), (LERCANIDIPINE HYDROCHLORIDE:Sinusitis:1), (LUMACAFTOR:Sinusitis:0.49), (LURASIDONE HYDROCHLORIDE:Sinusitis:0.53), (MESNA:Sinusitis:0.34), (METFORMIN HYDROCHLORIDEVILDAGLIPTIN:Sinusitis:0.25), (METHENAMINE HIPPURATE:Sinusitis:1.81), (METHENOLONE ENANTHATE:Sinusitis:0.27), (MOEXIPRIL HYDROCHLORIDE:Sinusitis:0.33), (MORPHINE SULFATE\NALTREXONE HYDROCHLORIDE:Sinusitis:0.38), (NADROPARIN CALCIUM:Sinusitis:0.66), (NALOXONE:Sinusitis:0.16), (NALOXONE HYDROCHLORIDETILIDINE HYDROCHLORIDE:Sinusitis:0.26), (NATEGLINIDE:Sinusitis:0.77), (NEPAFENAC:Sinusitis: 0.01), (NICARDIPINE:Sinusitis:0.03), (NIRAPARIB:Sinusitis:0.33), (OMEPRAZOLE SODIUM:Sinusitis:0.51), (OXYBUTYNINOXYBUTYNIN CHLORIDE:Sinusitis:0.17), (PALONOSETRON HYDROCHLORIDE:Sinusitis:0.26), (PATIROMER:Sinusitis:0.24), (PEMETREXED:Sinusitis:00.41), (PENTOSTATIN:Sinusitis:0.27), (PHENTERMINE HYDROCHLORIDE:Sinusitis:0.13), (PHENYTOIN SODIUM:Sinusitis:0.81), (PILOCARPINE HYDROCHLORIDE:Sinusitis:0.31), (PIOGLITAZONE:Sinusitis:0.2), (POLYETHYLENE GLYCOL 4000:Sinusitis:0.04), (PRAZOSIN HYDROCHLORIDE:Sinusitis:0.76), (PROTRIPTYLINE HYDROCHLORIDE:Sinusitis:0.74), (QUINACRINE:Sinusitis:0.44), (REGORAFENIB:Sinusitis: 0.84), (RIVASTIGMINE:Sinusitis:0.36), (ROFECOXIB:Sinusitis:0.1), (SAXAGLIPTIN HYDROCHLORIDE:Sinusitis:0.39), (SELEXIPAG:Sinusitis:0.25), (SILICON DIOXIDE:Sinusitis:0), (SODIUM PHOSPHATE, DIBASIC\SODIUM PHOSPHATE, MONOBASIC:Sinusitis:0.05), (STARCH, WHEAT:Sinusitis:0.16), (TAPENTADOL HYDROCHLORIDE:Sinusitis:0.23), (TAZOBACTAM:Sinusitis:0.26), (TEDUGLUTIDEWATER: Sinusitis:0.53), (TERCONAZOLE:Sinusitis:0.19), (TESTOSTERONE UNDECANOATE:Sinusitis:0.36), (TETRACYCLINE:Sinusitis:1.75), (THEOPHYLLINE:Sinusi tis:0.9), (TIGECYCLINE:Sinusitis:0.6), (TINZAPARIN:Sinusitis:0.37), (TIOTROPIUM:Sinusitis: 1.16), (TIPIRACIL HYDROCHLORIDETRIFLURIDINE:Sinusitis:0.11), (TOLTERODINE:Sinusitis:0.46), (VINC RISTINE SULFATE:Sinusitis:0.28), (ZILEUTON:Sinusitis:0.82), (.ALPHA.1-PROTEINASE INHIBITOR HUMAN:Sjogren's syndrome:0.3), (ACETAZOLAMIDE:Sjogren's syndrome:0.98), (AMANTADINE:Sjogren's syndrome:0.05), (ANASTROZOLE:Sjogren's syndrome:0.9), (ARIPIPRAZOLE:Sjogren's syndrome:0.12), (BENAZEPRILBENAZEPRIL HYDROCHLORIDE:Sjogren's syndrome:0.07), (BORTEZOMIB:Sjogren's syndrome:0.55), (BUDESONIDEFORMOTEROL FUMARATE:Sjogren's syndrome:0.26), (BUPRENORPHINE HYDROCHLORIDENALOXONE HYDROCHLORIDE:Sjogren's syndrome:0.4), (CALCITRIOL:Sjogren's syndrome:0.32), (CALCIUM\MAGNESIUM:Sjogren's syndrome:0.05), (CANAKINUMAB:Sjogren's syndrome:0.27), (CANDESARTAN CILEXETILHYDROCHLOROTHIAZIDE:Sjogren's syndrome:0.41), (CAPTOPRIL:Sjogren's syndrome:0.13), (CARFILZOMIB:Sjogren's syndrome:0.34), (CARVEDILOL PHOSPHATE:Sjogren's syndrome:0.71), (CEFTRIAXONE SODIUM:Sjogren's syndrome:0.06), (CETIRIZINE HYDROCHLORIDEPSEUDOEPHEDRINE HYDROCHLORIDE:Sjogren's syndrome:0.2), (CHLORTHALIDONE:Sjogren's syndrome:0.43), (CLINDAMYCIN:Sjogren's syndrome:0.19), (CLINDAMYCINCLINDAMYCIN PHOSPHATE:Sjogren's syndrome:1.11), (COLESEVELAM HYDROCHLORIDE:Sjogren's syndrome:0.18), (CORTISONEHYDROCORTISONE:Sjogren's syndrome:1.01), (DACLIZUMAB:Sjogren's syndrome:0.87), (DASATINIB:Sjogren's syndrome:0.51), (DESONIDE:Sjogren's syndrome:0.09), (DOCUSATE:Sjogren's syndrome:0.24), (DOCUSATE CALCIUM:Sjogren's syndrome:0.39), (DOXAZOSIN MESYLATE:Sjogren's syndrome:0.08), (DOXAZOSINDOXAZOSIN MESYLATE:Sjogren's syndrome:0.05), (EMPAGLIFLOZINLINAGLIPTIN:Sjogren's syndrome:0.13), (ENALAPRIL MALEATE:Sjogren's syndrome:0.66), (ERYTHROPOIETIN:Sjogren's syndrome:0.14), (ESTROGENS:Sjogren's syndrome:0.25), (ESZOPICLONE:Sjogren's syndrome:1.24), (EVEROLIMUS:Sjogren's syndrome:0.1), (FINGOLIMOD HYDROCHLORIDE:Sjogren's syndrome:0.59), (FLUOCINONIDE:Sjogren's syndrome:0.14), (FLUTICASONE:Sjogren's syndrome:0.79), (FLUTICASONE FUROATE:Sjogren's syndrome:0.2), (GLUCOSAMINE:Sjogren's syndrome:0.19), (GLYBURIDE:Sjogren's syndrome:0.44), (GUAIFENESIN:Sjogren's syndrome:0.29), (HALOPERIDOL:Sjogren's syndrome:0.08), (HYDRALAZINE HYDROCHLORIDE:Sjogren's syndrome:0.47), (HYDROCHLOROTHIAZIDELISINOPRIL:Sjogren's syndrome:0.87), (HYDROCORTISONE ACETATE:Sjogren's syndrome:0.06), (IBRUTINIB:Sjogren's syndrome:0.92), (INDAPAMIDE:Sjogren's syndrome:0.41), (INDOMETHACIN:Sjogren's syndrome:0.68), (IPRATROPIUM:Sjogren's syndrome:0.77), (ISOSORBIDE MONONITRATE:Sjogren's syndrome:1.46), (KETOROLACKETOROLAC TROMETHAMINE:Sjogren's syndrome:0.06), (LABETALOLLABETALOL HYDROCHLORIDE:Sjogren's syndrome:0.45), (LACTASE:Sjogren's syndrome:0.01), (LATANOPROSTTIMOLOL:Sjogren's syndrome:0.27), (LEDIPASVIRSOFOSBUVIR:Sjogren's syndrome:0.25), (LERCANIDIPINE:Sjogren's syndrome:0.11), (METHENAMINE HIPPURATE:Sjogren's syndrome:0.34), (METOLAZONE:Sjogren's syndrome:0.37), (MOXIFLOXACIN:Sjogren's syndrome:0.02), (MUPIROCINMUPIROCIN CALCIUM:Sjogren's syndrome:0.01), (NEBIVOLOL:Sjogren's syndrome:0.8), (NILOTINIB:Sjogren's syndrome:0.32), (OLANZAPINE:Sjogren's syndrome:0.71), (OLODATEROL HYDROCHLORIDETIOTROPIUM BROMIDE MONOHYDRATE:Sjogren's syndrome:0.09), (PANTOPRAZOLE MAGNESIUM:Sjogren's syndrome:0.84), (PAZOPANIB HYDROCHLORIDE:Sjogren's syndrome:0.14), (PEGINTERFERON BETA-A:Sjogren's syndrome:0.18), (PENICILLIN:Sjogren's syndrome:0.57), (PHENYTOIN:Sjogren's syndrome:0.79), (PRIMIDONE:Sjogren's syndrome:1.04), (ROFLUMILAST:Sjogren's syndrome:0.19), (SCOPOLAMINE:Sjogren's syndrome:0.01), (SENNA LEAFSENNOSIDESSENNOSIDES AAND B:Sjogren's syndrome:0.13), (SOTALOL:Sjogren's syndrome:0.4), (TETRACYCLINE:Sjogren's syndrome:0.13), (TIMOLOL MALEATE:Sjogren's syndrome:0.29), (UMECLIDINIUM BROMIDEVILANTEROL TRIFENATATE:Sjogren's syndrome:0.68), (VARENICLINE TARTRATE:Sjogren's syndrome:1.22), (ALBUTEROL:Skin cancer:0.12), (ANASTROZOLE:Skin cancer:0.38), (BUDESONIDE:Skin cancer:0.56), (CERTOLIZUMAB PEGOL:Skin cancer:0.22), (DEXLANSOPRAZOLE:Skin cancer:0.79), (DIVALPROEX SODIUM:Skin cancer:0.19), (EVOLOCUMAB:Skin cancer:0.53), (HYDROXYCHLOROQUINE SULFATE:Skin cancer:0.06), (INSULIN ASPART:Skin cancer:0.13), (METRONIDAZOLE:Skin cancer:0.79), (PANTOPRAZOLE:Skin cancer:0.59), (PRAMIPEXOLE DIHYDROCHLORIDE:Skin cancer:0.07), (PRAVASTATIN:Skin cancer:0.2), (ACETAMINOPHENTRAMADOL:Skin disorder:0.06), (ACETAZOLAMIDE:Skin disorder:0.33), (ALEMTUZUMAB:Skin disorder:0.41), (AMPHOTERICIN B:Skin disorder:0.1), (AZELASTINE:Skin disorder:0.1), (BENAZEPRILBENAZEPRIL HYDROCHLORIDE:Skin disorder:0.32), (BICALUTAMIDE:Skin disorder:0.03), (BORTEZOMIB:Skin disorder:0.15), (BUPRENORPHINE HYDROCHLORIDE:Skin disorder:0.04), (BUPRENORPHINE HYDROCHLORIDENALOXONE HYDROCHLORIDE:Skin disorder:0.03), (CALCITONIN SALMON:Skin disorder:0.38), (CALCIUM ACETATE:Skin disorder:0.47), (CALCIUM\MAGNESIUM\ZINC:Skin disorder:0.24), (CANAGLIFLOZINMETFORMIN HYDROCHLORIDE:Skin disorder:0.52), (CANAKINUMAB:Skin disorder:0.09), (CANDESARTAN CILEXETILHYDROCHLOROTHIAZIDE:Skin disorder:0.88), (CAPECITABINE:Skin disorder:0.07), (CARVEDILOL PHOSPHATE:Skin disorder:0.8), (CEFAZOLIN:Skin disorder:0.09), (CEFTRIAXONE SODIUM:Skin disorder:0.31), (CIMETIDINE:Skin disorder:0.68), (CLARITHROMYCIN:Skin disorder:0.15), (CORTICOTROPIN:Skin disorder:0.15), (DAPAGLIFLOZIN PROPANEDIOL:Skin disorder:0.76), (DASATINIB:Skin disorder:0.95), (DIVALPROEX SODIUM:Skin disorder:0.91), (DOXEPIN HYDROCHLORIDE:Skin disorder:0.26), (DULAGLUTIDE:Skin disorder:0.35), (EDOXABAN TOSYLATE:Skin disorder:0.48), (EFINACONAZOLE:Skin disorder:0.37), (ELETRIPTAN HYDROBROMIDE:Skin disorder:1.1), (EMPAGLIFLOZINLINAGLIPTIN:Skin disorder:0.68), (ENOXAPARIN:Skin disorder:1.02), (EPLERENONE:Skin disorder:0.08), (ESOMEPRAZOLE:Skin disorder:0.82), (ESOMEPRAZOLE MAGNESIUMNAPROXEN:Skin disorder:0.38), (ESTRADIOLNORETHINDRONE ACETATE:Skin disorder:0.47), (ESTROGENS, CONJUGATEDMEDROXYPROGESTERONE ACETATE:Skin disorder:0.96), (FENOFIBRIC ACID:Skin disorder:0.02), (FENTANYL CITRATE:Skin disorder:0.17), (FLUDROCORTISONE ACETATE:Skin disorder:0.02), (FLUTICASONE FUROATESALMETEROL XINAFOATE:Skin disorder:0.01), (FLUTICASONE FUROATEVILANTEROL TRIFENATATE:Skin disorder:0.62), (FORMOTEROL FUMARATE:Skin disorder:0.18), (GLYBURIDE:Skin disorder:0.48), (HYDROCHLOROTHIAZIDEIRBESARTAN:Skin disorder:0.52), (HYDROCHLOROTHIAZIDEVALSARTAN:Skin disorder:0.45), (HYDROXYCHLOROQUINE DIPHOSPHATE:Skin disorder:0.3), (HYDROXYZINE PAMOATE:Skin disorder:1.29), (HYOSCYAMINE:Skin disorder:0.31), (IMIPRAMINE HYDROCHLORIDE:Skin disorder:0.74), (IPRATROPIUM:Skin disorder:0.65), (LAPATINIB DITOSYLATE:Skin disorder:0.14), (LEUCOVORIN CALCIUM:Skin disorder:1.02), (LEVALBUTEROL HYDROCHLORIDE:Skin disorder:0.67), (METHENAMINE HIPPURATE:Skin disorder:0.67), (METHIMAZOLE:Skin disorder:0.23), (METHYLPREDNISOLONE ACETATE:Skin disorder:0.95), (MICONAZOLE NITRATE:Skin disorder:0.04), (MOXIFLOXACIN HYDROCHLORIDE:Skin disorder:1.49), (NADOLOL:Skin disorder:0.46), (NATEGLINIDE:Skin disorder:0.13), (NEBIVOLOL:Skin disorder:0.11), (NICOTINE:Skin disorder:0.57), (NORETHINDRONE:Skin disorder:0.47), (OLAPARIB:Skin disorder:0), (OXCARBAZEPINE:Skin disorder:1.87), (PANTOPRAZOLE MAGNESIUM:Skin disorder:1.2), (PAZOPANIB HYDROCHLORIDE:Skin disorder:0.75), (PEGFILGRASTIM:Skin disorder:0.66), (PEGINTERFERON BETA-1A:Skin disorder:0.45), (PENICILLIN:Skin disorder:0.63), (PENTOSAN POLYSULFATE SODIUM:Skin disorder:0.32), (PIROXICAM:Skin disorder:0.21), (PRAMIPEXOLE DIHYDROCHLORIDE:Skin disorder:0.84), (PRAZOSIN:Skin disorder:0.05), (PRAZOSIN HYDROCHLORIDE:Skin disorder:0.03), (RALOXIFENE HYDROCHLORIDE:Skin disorder:0.16), (ROFLUMILAST:Skin disorder:0.77), (SACUBITRILVALSARTAN:Skin disorder:0.92), (SOLIFENACIN SUCCINATE:Skin disorder:0.51), (SULFAMETHOXAZOLE:Skin disorder:0.61), (TERBUTALINE SULFATE:Skin disorder:0.67), (TOCILIZUMAB:Skin disorder:0.55), (UMECLIDINIUM BROMIDEVILANTEROL TRIFENATATE:Skin disorder:0.83), (VALGANCICLOVIR HYDROCHLORIDE:Skin disorder:0), (VEMURAFENIB:Skin disorder:0.04), (ACARBOSE:Skin infection:0.06), (ACETAMINOPHENBUTALBITALCAFFEINE:Skin infection:0.25), (ACETAMINOPHENOXYCODONEOXYCODONE HYDROCHLORIDE:Skin infection:0.06), (ALBUTEROL SULFATEIPRATROPIUM BROMIDE:Skin infection:0.8), (ALCOHOL:Skin infection:0.36), (ANASTROZOLE:Skin infection:1.24), (BISACODYL OR DOCUSATE SODIUM:Skin infection:0.76), (BORTEZOMIB:Skin infection:0.7), (BUPRENORPHINE HYDROCHLORIDE:Skin infection:0.39), (BUPRENORPHINE HYDROCHLORIDENALOXONE HYDROCHLORIDE:Skin infection:0.04), (CALCIUM ACETATE:Skin infection:0.44), (CALCIUM CITRATE:Skin infection:0.16), (CANAKINUMAB:Skin infection:0.34), (CANDESARTAN CILEXETILHYDROCHLOROTHIAZIDE:Skin infection:0.68), (CARISOPRODOL:Skin infection:0.09), (CARVEDILOL PHOSPHATE:Skin infection:0.11), (CEFOTAXIME SODIUM:Skin infection:0.7), (DACLIZUMAB:Skin infection:0.13), (DESLORATADINE:Skin infection:0.48), (DEXAMETHASONE SODIUM PHOSPHATE:Skin infection:0.02), (DUTASTERIDE:Skin infection:0.26), (ECONAZOLE:Skin infection:0.12), (ELETRIPTAN HYDROBROMIDE:Skin infection:0.34), (ETORICOXIB:Skin infection:0.04), (EVEROLIMUS:Skin infection:1.05), (EVOLOCUMAB:Skin infection:0.62), (EXEMESTANE:Skin infection:0.01), (FESOTERODINE FUMARATE:Skin infection:0.13), (FLECAINIDE:Skin infection:0.5), (FLUOXETINEFLUOXETINE HYDROCHLORIDE:Skin infection:0.4), (FLUTICASONE:Skin infection:0.39), (FLUTICASONE FUROATE:Skin infection:0.19), (FORMOTEROL FUMARATE:Skin infection:0.21), (FORMOTEROL FUMARATE DIHYDRATE\MOMETASONE FUROATE:Skin infection:0.1), (GLYCOPYRRONIUMINDACATEROL:Skin infection:0.12), (HYDROCHLOROTHIAZIDEIRBESARTAN:Skin infection:0.09), (HYDROCHLOROTHIAZIDELISINOPRIL:Skin infection:1.05), (ILOPROST:Skin infection:0.06), (IMIPENEM:Skin infection:0.54), (IMIPRAMINE HYDROCHLORIDE:Skin infection:0.71), (INDAPAMIDEPERINDOPRIL:Skin infection:0.34), (LERCANIDIPINE:Skin infection:0.45), (LERCANIDIPINE HYDROCHLORIDE:Skin infection:0.21), (LEUCOVORIN CALCIUM:Skin infection:1.32), (LIDOCAINEPRILOCAINE:Skin infection:0.47), (LITHIUM CARBONATE:Skin infection:0.74), (LOVASTATIN:Skin infection:0.82), (METAXALONE:Skin infection:0.21), (METHADONE HYDROCHLORIDE:Skin infection:0.44), (METHYLPREDNISOLONE ACETATE:Skin infection:0.47), (NALOXONE HYDROCHLORIDEOXYCODONE HYDROCHLORIDE:Skin infection:0.42), (NEBIVOLOL:Skin infection:0.72), (NEBIVOLOL HYDROCHLORIDE:Skin infection:0.33), (NORTRIPTYLINE:Skin infection:0.3), (PANTOPRAZOLE MAGNESIUM:Skin infection:0.6), (PAZOPANIB HYDROCHLORIDE:Skin infection:0.06), (PEMETREXED:Skin infection:0.05), (PEMETREXED DISODIUM:Skin infection:0.04), (PHENOBARBITAL:Skin infection:1.05), (PHENYTOIN SODIUM:Skin infection:0.33), (PIROXICAM:Skin infection:0.02), (PRAMIPEXOLE DIHYDROCHLORIDE:Skin infection:0.31), (PRAVASTATINPRAVASTATIN SODIUM:Skin infection:0.05), (PROPRANOLOL HYDROCHLORIDE:Skin infection:0.67), (RIZATRIPTAN BENZOATE:Skin infection:0.3), (ROFLUMILAST:Skin infection:0.5), (SACUBITRILVALSARTAN:Skin infection:0.9), (SCOPOLAMINE:Skin infection:0.29), (SULFAMETHOXAZOLE:Skin infection:0.89), (SUMATRIPTANSUMATRIPTAN SUCCINATE:Skin infection:0.17), (TELMISARTAN:Skin infection:0.81), (TERIFLUNOMIDE:Skin infection:0.53), (THALIDOMIDE:Skin infection:0.81), (TIMOLOL:Skin infection:0.13), (TIOTROPIUM:Skin infection:0.15), (TOCILIZUMAB:Skin infection:0.52), (TOFACITINIB CITRATE:Skin infection:0.38), (VALGANCICLOVIR HYDROCHLORIDE:Skin infection:0.45), (VINCRISTINE SULFATE:Skin infection:0.52), (BISOPROLOL FUMARATE:Skin irritation:0.07), (CALCIUM CARBONATECHOLECALCIFEROL:Skin irritation:0.01), (DILTIAZEM HYDROCHLORIDE:Skin irritation:0), (DULOXETINE:Skin irritation:0.22), (ENALAPRIL:Skin irritation:0.61), (EPINEPHRINE:Skin irritation:0), (ESCITALOPRAM OXALATE:Skin irritation:0.75), (EVOLOCUMAB:Skin irritation:0.2), (FINGOLIMOD HYDROCHLORIDE:Skin irritation:0.03), (IRON:Skin irritation:0.31), (LISDEXAMFETAMINE DIMESYLATE:Skin irritation:0.07), (POTASSIUM:Skin irritation:0.54), (SACUBITRILVALSARTAN:Skin irritation:0.01), (ALENDRONIC ACID:Skin lesion:0.09), (AZITHROMYCIN ANHYDROUS:Skin lesion:0.14), (BUMETANIDE:Skin lesion:0.52), (CANDESARTAN:Skin lesion:0.12), (CANDESARTAN CILEXETILHYDROCHLOROTHIAZIDE:Skin lesion:0.12), (CLINDAMYCINCLINDAMYCIN PHOSPHATE:Skin lesion:1.15), (CORTISONEHYDROCORTISONE:Skin lesion:0.03), (DASATINIB:Skin lesion:0.18), (DICLOFENAC:Skin lesion:0.43), (DILTIAZEM:Skin lesion:0.62), (DILTIAZEM HYDROCHLORIDE:Skin lesion:0.9), (ESOMEPRAZOLE:Skin lesion:0.39), (EVOLOCUMAB:Skin lesion:0.32), (FENOFIBRATE:Skin lesion:0.23), (HYDROCHLOROTHIAZIDELISINOPRIL:Skin lesion:0.13), (IDELALISIB:Skin lesion:0.06), (INSULIN HUMAN:Skin lesion:0.11), (LABETALOLLABETALOL HYDROCHLORIDE:Skin lesion:0.04), (LERCANIDIPINE:Skin lesion:0.16), (METHYLPREDNISOLONE ACETATE:Skin lesion:0.24), (METHYLPREDNISOLONE SODIUM SUCCINATE:Skin lesion:0.39), (METRONIDAZOLEMETRONIDAZOLE HYDROCHLORIDE:Skin lesion:0.17), (OXCARBAZEPINE:Skin lesion:0.82), (OXYGEN:Skin lesion:0.33), (PANTOPRAZOLE MAGNESIUM:Skin lesion:0.02), (PHENOBARBITAL:Skin lesion:0.46), (PRAVASTATIN:Skin lesion:0.44), (PRIMIDONE:Skin lesion:0.11), (PROMETHAZINEPROMETHAZINE HYDROCHLORIDE:Skin lesion:0.26), (RALOXIFENE HYDROCHLORIDE:Skin lesion:0.24), (RANOLAZINE:Skin lesion:0.16), (SACUBITRILVALSARTAN:Skin lesion:0.08), (TEMAZEPAM:Skin lesion:0.03), (TOFACITINIB CITRATE:Skin lesion:0.38), (TORSEMIDE:Skin lesion:0.36), (ZONISAMIDE:Skin lesion:0.07), (ARTEMISIA VULGARIS POLLEN:Skin test:0.24), (CHENOPODIUM ALBUM POLLEN:Skin test:0.42), (ETIMICIN SULFATE:Skin test:0.01), (ULMUS AMERICANA POLLEN:Skin test:0.52), (ACARBOSE:Skin ulcer:0.11), (ACETAMINOPHENBUTALBITALCAFFEINE:Skin ulcer:0.37), (ACETAMINOPHENOXYCODONEOXYCODONE HYDROCHLORIDE:Skin ulcer:0.01), (ANASTROZOLE:Skin ulcer:0.64), (AZITHROMYCIN DIHYDRATE:Skin ulcer:0.21), (BENAZEPRILBENAZEPRIL HYDROCHLORIDE:Skin ulcer:0.07), (BENZONATATE:Skin ulcer:0), (BISACODYL OR DOCUSATE SODIUM:Skin ulcer:0.72), (BORTEZOMIB:Skin ulcer:0.53), (BUPRENORPHINE HYDROCHLORIDE:Skin ulcer:0.02), (CALCIUM CARBONATEVITAMIN D:Skin ulcer:0.01), (CARVEDILOL PHOSPHATE:Skin ulcer:0.53), (CEFOTAXIME SODIUM:Skin ulcer:0.06), (CILASTATIN SODIUMIMIPENEM:Skin ulcer:0.05), (CORTISONEHYDROCORTISONE:Skin ulcer:0.35), (DACLIZUMAB:Skin ulcer:0.05), (DALTEPARIN SODIUM:Skin ulcer:0.09), (DANAZOL:Skin ulcer:0.21), (DAPAGLIFLOZIN PROPANEDIOL:Skin ulcer:0.1), (DASATINIB:Skin ulcer:0.64), (DEVICE:Skin ulcer:0.22), (DIAZEPAM:Skin ulcer:0.38), (DIMENHYDRINATE:Skin ulcer:0.2), (DOFETILIDE:Skin ulcer:0.06), (EDOXABAN TOSYLATE:Skin ulcer:0.69), (ELETRIPTAN HYDROBROMIDE:Skin ulcer:0.22), (EPINEPHRINE:Skin ulcer:0.84), (EPLERENONE:Skin ulcer:0.19), (ESTROGENS, CONJUGATEDMEDROXYPROGESTERONE ACETATE:Skin ulcer:0.04), (EVOLOCUMAB:Skin ulcer:0.8), (FESOTERODINE FUMARATE:Skin ulcer:0.2), (GLYCOPYRRONIUMINDACATEROL:Skin ulcer:0.02), (HYDROCHLOROTHIAZIDELISINOPRIL:Skin ulcer:0.76), (HYDROCORTISONE ACETATE:Skin ulcer:0.04), (HYDROXYCHLOROQUINE DIPHOSPHATE:Skin ulcer:0.09), (HYOSCYAMINE:Skin ulcer:0.13), (INDAPAMIDEPERINDOPRIL:Skin ulcer:0.44), (INDOMETHACIN:Skin ulcer:0.2), (LERCANIDIPINE:Skin ulcer:0.98), (LEVALBUTEROL HYDROCHLORIDE:Skin ulcer:0.86), (METAXALONE:Skin ulcer:0.42), (METHIMAZOLE:Skin ulcer:0.18), (METHYLPHENIDATE HYDROCHLORIDE:Skin ulcer:0.05), (METHYLPREDNISOLONE ACETATE:Skin ulcer:0.03), (MODAFINIL:Skin ulcer:0.1), (NABUMETONE:Skin ulcer:0.02), (NALOXONE HYDROCHLORIDEOXYCODONE HYDROCHLORIDE:Skin ulcer:0.31), (OLANZAPINE:Skin ulcer:0.09), (OXCARBAZEPINE:Skin ulcer:0.38), (PANTOPRAZOLE MAGNESIUM:Skin ulcer:0.57), (PAROXETINEPAROXETINE HYDROCHLORIDE:Skin ulcer:0.1), (PEGFILGRASTIM:Skin ulcer:0.1), (PENICILLIN:Skin ulcer:0.23), (PONATINIB:Skin ulcer:0.31), (PRIMIDONE:Skin ulcer:0.38), (RIOCIGUAT:Skin ulcer:0.28), (SACUBITRILVALSARTAN:Skin ulcer:0.43), (SELEXIPAG:Skin ulcer:0.18), (SENNOSIDES:Skin ulcer:0.21), (SEVELAMER HYDROCHLORIDE:Skin ulcer:0.42), (SULFAMETHOXAZOLE:Skin ulcer:0.44), (SUMATRIPTAN:Skin ulcer:0.08), (TELMISARTAN:Skin ulcer:1.11), (TERBINAFINE HYDROCHLORIDE:Skin ulcer:0.05), (TERIFLUNOMIDE:Skin ulcer:0.41), (TETRACYCLINE:Skin ulcer:0.21), (THALIDOMIDE:Skin ulcer:0.24), (TIMOLOL:Skin ulcer:0.25), (TIOTROPIUM:Skin ulcer:0.1), (TIZANIDINE HYDROCHLORIDE:Skin ulcer:0.45), (TOFACITINIB CITRATE:Skin ulcer:0.02), (UMECLIDINIUM BROMIDEVILANTEROL TRIFENATATE:Skin ulcer:0.08), (VALGANCICLOVIR HYDROCHLORIDE:Skin ulcer:0.66), (VARENICLINE TARTRATE:Skin ulcer:0.64), (VERAPAMIL HYDROCHLORIDE:Skin ulcer:1.2), (ARMODAFINIL:Skin wrinkling:0.78), (ATROPINE SULFATEDIPHENOXYLATE HYDROCHLORIDE:Skin wrinkling:0.27), (CALCIUM CARBONATECHOLECALCIFEROL:Skin wrinkling:0.55), (CERTOLIZUMAB PEGOL:Skin wrinkling:0.86), (CLINDAMYCINCLINDAMYCIN PHOSPHATE:Skin wrinkling:0.08), (DACLIZUMAB:Skin wrinkling:0.12), (DALFAMPRIDINE:Skin wrinkling:0.33), (DILTIAZEM:Skin wrinkling:0.24), (DIPHENHYDRAMINE:Skin wrinkling:0.45), (DIVALPROEX SODIUM:Skin wrinkling:0.3), (EVOLOCUMAB:Skin wrinkling:0.7), (FERROUS SULFATE:Skin wrinkling:0.06), (HYDROMORPHONE HYDROCHLORIDE:Skin wrinkling:0.04), (HYDROXYCHLOROQUINE DIPHOSPHATE:Skin wrinkling:0.06), (HYDROXYZINE PAMOATE:Skin wrinkling:0.14), (HYOSCYAMINE:Skin wrinkling:0.1), (IBRUTINIB:Skin wrinkling:0.17), (INDOMETHACIN:Skin wrinkling:0.28), (INSULIN GLARGINE:Skin wrinkling:0.18), (MEDROXYPROGESTERONE ACETATE:Skin wrinkling:0.02), (METHYLPREDNISOLONE ACETATE:Skin wrinkling:0.14), (NORETHINDRONE:Skin wrinkling:0.01), (PANTOPRAZOLE MAGNESIUM:Skin wrinkling:0.01), (PRAVASTATIN SODIUM:Skin wrinkling:0.1), (PRIMIDONE:Skin wrinkling:0.24), (PROMETHAZINE HYDROCHLORIDE:Skin wrinkling:0), (PROMETHAZINEPROMETHAZINE HYDROCHLORIDE:Skin wrinkling:0.22), (RIZATRIPTAN BENZOATE:Skin wrinkling:0.58), (TAMSULOSIN:Skin wrinkling:0.22), (TETRACYCLINE:Skin wrinkling:0.2), (TIOTROPIUM BROMIDE MONOHYDRATE:Skin wrinkling:0.14), (TIZANIDINE HYDROCHLORIDE:Skin wninkling:0.51), (TOFACITINIB CITRATE:Skin wrinkling:0.05), (UMECLIDINIUM BROMIDEVILANTEROL TRIFENATATE:Skin wrinkling:0.11), (VARENICLINE TARTRATE:Skin wrinkling:0.84), (.ALPHA.1-PROTEINASE INHIBITOR HUMAN:Sleep apnoea syndrome:0.18), (ALBIGLUTIDE:Sleep apnoea syndrome:0.54), (ALCOHOL:Sleep apnoea syndrome:0.2), (ALENDRONIC ACID:Sleep apnoea syndrome:0.01), (AMLODIPINE BESYLATEHYDROCHLOROTHIAZIDEOLMESARTAN MEDOXOMIL:Sleep apnoea syndrome:0.06), (BIFIDOBACTERIUM LONGUM SUBSP. INFANTIS:Sleep apnoea syndrome:0.19), (BROMAZEPAM:Sleep apnoea syndrome:0.12), (BUPRENORPHINE HYDROCHLORIDE:Sleep apnoea syndrome:0.37), (CANAGLIFLOZINMETFORMIN HYDROCHLORIDE:Sleep apnoea syndrome:0.19), (CANDESARTAN:Sleep apnoea syndrome:1.03), (CANDESARTAN CILEXETILHYDROCHLOROTHIAZIDE:Sleep apnoea syndrome:0.95), (CARVEDILOL PHOSPHATE:Sleep apnoea syndrome:1.15), (CETIRIZINE HYDROCHLORIDEPSEUDOEPHEDRINE HYDROCHLORIDE:Sleep apnoea syndrome:0.43), (CHOLESTYRAMINE:Sleep apnoea syndrome:1.3), (CILOSTAZOL:Sleep apnoea syndrome:0.02), (CIMETIDINE:Sleep apnoea syndrome:0.14), (CLOBAZAM:Sleep apnoea syndrome:0.05), (CLONIDINE HYDROCHLORIDE:Sleep apnoea syndrome:0.24), (CLOZAPINE:Sleep apnoea syndrome:0.14), (COD LIVER OIL:Sleep apnoea syndrome:0.14), (CODEINE:Sleep apnoea syndrome:0.19), (DABIGATRAN:Sleep apnoea syndrome:0.13), (DACLIZUMAB:Sleep apnoea syndrome:0.75), (DARBEPOETIN ALFA:Sleep apnoea syndrome:0.87), (DASATINIB:Sleep apnoea syndrome:0.82), (DIMENHYDRINATE:Sleep apnoea syndrome:0.25), (DOCUSATE CALCIUM:Sleep apnoea syndrome:0.57), (DOFETILIDE:Sleep apnoea syndrome:0.66), (DOXAZOSIN MESYLATE:Sleep apnoea syndrome:0.45), (EDOXABAN TOSYLATE:Sleep apnoea syndrome:0), (EMPAGLIFLOZINLINAGLIPTIN:Sleep apnoea syndrome:0.24), (ESTROGENS, CONJUGATEDMEDROXYPROGESTERONE ACETATE:Sleep apnoea syndrome:0.28), (ETORICOXIB:Sleep apnoea syndrome:0.19), (EVOLOCUMAB:Sleep apnoea syndrome:2.01), (FORMOTEROL FUMARATE\MOMETASONE FUROATE:Sleep apnoea syndrome:0.19), (GLICLAZIDE:Sleep apnoea syndrome:0.4), (GUANFACINE HYDROCHLORIDE:Sleep apnoea syndrome:0.17), (HALOPERIDOL:Sleep apnoea syndrome:0.23), (HERBALSTURMERIC:Sleep apnoea syndrome:0.29), (HYDROCHLOROTHIAZIDETELMISARTAN:Sleep apnoea syndrome:0.11), (HYOSCYAMINE:Sleep apnoea syndrome:0.83), (IBRUTINIB:Sleep apnoea syndrome:0.93), (IMIPRAMINE HYDROCHLORIDE:Sleep apnoea syndrome:0.67), (INDAPAMIDE:Sleep apnoea syndrome:0.59), (INDAPAMIDEPERINDOPRIL:Sleep apnoea syndrome:0.33), (INFLUENZA VIRUS VACCINE:Sleep apnoea syndrome:0.31), (LERCANIDIPINE HYDROCHLORIDE:Sleep apnoea syndrome:0.16), (LIDOCAINE HYDROCHLORIDE:Sleep apnoea syndrome:0.52), (LOMITAPIDE MESYLATE:Sleep apnoea syndrome:0.41), (MEPOLIZUMAB:Sleep apnoea syndrome:0.08), (METHYLPREDNISOLONE ACETATE:Sleep apnoea syndrome:0.49), (METRONIDAZOLEMETRONIDAZOLE HYDROCHLORIDE:Sleep apnoea syndrome:0.6), (NALOXONE HYDROCHLORIDEOXYCODONE HYDROCHLORIDE:Sleep apnoea syndrome:0.57), (NATEGLINIDE:Sleep apnoea syndrome:0.07), (PANTOPRAZOLE MAGNESIUM:Sleep apnoea syndrome:1.28), (PATIROMER:Sleep apnoea syndrome:0.2), (PAZOPANIB HYDROCHLORIDE:Sleep apnoea syndrome:0.02), (PEGINTERFERON BETA-1A:Sleep apnoea syndrome:0.79), (PENICILLIN:Sleep apnoea syndrome:0.35), (PIROXICAM:Sleep apnoea syndrome:0.12), (PONATINIB:Sleep apnoea syndrome:0), (PRAZOSIN:Sleep apnoea syndrome:0.4), (PRAZOSIN HYDROCHLORIDE:Sleep apnoea syndrome:0.01), (PREDNISOLONE ACETATE:Sleep apnoea syndrome:0.13), (RABEPRAZOLE:Sleep apnoea syndrome:0.49), (RISEDRONATE SODIUM:Sleep apnoea syndrome:0.32), (RIZATRIPTAN:Sleep apnoea syndrome:0.1), (SCOPOLAMINE:Sleep apnoea syndrome:0.36), (TERBINAFINE HYDROCHLORIDE:Sleep apnoea syndrome:0.01), (TERBUTALINE SULFATE:Sleep apnoea syndrome:0.24), (TETRACYCLINE:Sleep apnoea syndrome:0.65), (THALIDOMIDE:Sleep apnoea syndrome:0.1), (TOCILIZUMAB:Sleep apnoea syndrome:0.71), (VITAMIN A:Sleep apnoea syndrome:0.27), (ZAFIRLUKAST:Sleep apnoea syndrome:0.01), (.ALPHA.-TOCOPHEROLVITAMINS:Sleep disorder:0.83), (.BETA.-HYDROXYISOVALERIC ACIDARGININEGLUTAMINE:Sleep disorder:0.02), (ABACAVIR:Sleep disorder:0.09), (ACETAMINOPHEN\CAFFEINEPROPOXYPHENE:Sleep disorder:0.05), (ACETAMINOPHENCHLORPHENIRAMINE MALEATEPSEUDOEPHEDRINE HYDROCHLORIDE:Sleep disorder:0.87), (ACETAMINOPHENMETHOCARBAMOL:Sleep disorder:1.11), (ALBUTEROLGUAIFENESIN:Sleep disorder:0.18), (ALBUTEROLIPRATROPIUM BROMIDE:Sleep disorder:0.41), (ALCOHOLALUMINUM CHLORIDE:Sleep disorder:0.5), (ALFALFA:Sleep disorder:0.4), (ALISKIRENHYDROCHLOROTHIAZIDE:Sleep disorder:0.04), (ALLANTOINBENZOCAINECAMPHOR (NATURAL)DIMETHICONEMENTHOL:Sleep disorder:0.23), (ALOGLIPTIN BENZOATE:Sleep disorder:0.09), (ALUMINUM CHLORIDE:Sleep disorder:0.2), (ALUMINUM HYDROXIDE:Sleep disorder:0.15), (ALUMINUM HYDROXIDEMAGNESIUM HYDROXIDESIMETHICONE:Sleep disorder:0.94), (AMILORIDE HYDROCHLORIDEFUROSEMIDE:Sleep disorder:0.64), (AMINOBENZOIC ACIDCALCIUM PANTOTHENATECYSTINEKERATINTHIAMINE MONONITRATE:Sleep disorder:0.44), (AMINOPHYLLINE:Sleep disorder:0.66), (AMINOSALICYLIC ACID:Sleep disorder:0.27), (AMOBARBITAL SODIUM:Sleep disorder:0.11), (AMPHETAMINE ASPARTATE MONOHYDRATEAMPHETAMINE SULFATEDEXTROAMPHETAMINE SACCHARATEDEXTROAMPHETAMINE SULFATE:Sleep disorder:0.02), (AMPICILLIN SODIUM:Sleep disorder:0.1), (ANAGRELIDE:Sleep disorder:0.07), (ANAGRELIDE HYDROCHLORIDE:Sleep disorder:0.71), (ANHYDROUS CITRIC ACIDMAGNESIUM OXIDE\SODIUM PICOSULFATE:Sleep disorder:0.14), (ARCTOSTAPHYLOS UVA-URSI LEAFURTICA DIOICA:Sleep disorder:0.14), (ASCORBIC ACIDFERROUS SULFATEFOLIC ACIDPYRIDOXINE HYDROCHLORIDE\RIBOFLAVINTHIAMINE MONONITRATE:Sleep disorder:0.24), (ASENAPINE:Sleep disorder:0.21), (ASPARAGINASE:Sleep disorder:0.34), (ASPIRIN\CAFFEINESALICYLAMIDE:Sleep disorder:0.81), (ASUNAPREVIR:Sleep disorder:0.53), (ATAZANAVIR:Sleep disorder:0.11), (AVOCADO OILSOYBEAN OIL:Sleep disorder:0.16), (AZITHROMYCIN OR METHOTREXATE SODIUM:Sleep disorder:0.8), (AZOSEMIDE:Sleep disorder:0.53), (BACLOFENCYCLOBENZAPRINEDICLOFENACGABAPENTINTETRAC AINE:Sleep disorder:0.07), (BECLOMETHASONE DIPROPIONATEFORMOTEROL:Sleep disorder:0.73), (BELATACEPT:Sleep disorder:0.97), (BENZOIC ACIDHYOSCYAMINE SULFATEMETHENAMINEMETHYLENE BLUEPHENYL SALICYLATE:Sleep disorder:0.27), (BEPOTASTINEBEPOTASTINE BESILATE:Sleep disorder:0.02), (BETA GLUCAN:Sleep disorder:0.07), (BETAMETHASONE ACETATEBETAMETHASONE SODIUM PHOSPHATE:Sleep disorder:0.19), (BETAMETHASONE DIPROPIONATECALCIPOTRIENE HYDRATE:Sleep disorder:0.23), (BETAMETHASONECALCIPOTRIENE:Sleep disorder:1.68), (BIFIDOBACTERIUM SPP.:Sleep disorder:0.26), (BORIC ACID:Sleep disorder:0.01), (BORON:Sleep disorder:0.46), (BRIVARACETAM:Sleep disorder:0.88), (BUDESONIDEFORMOTEROL:Sleep disorder:1.38), (BUTORPHANOL:Sleep disorder:0.76), (CABOZANTINIB:Sleep disorder:0.25), (CAFFEINEISOMETHEPTENEMETHIMAZOLE:Sleep disorder:0.21), (CAJUPUT OIL\EUCALYPTUS OILPEPPERMINT OIL:Sleep disorder:0.58), (CALCIUM ASCORBATE:Sleep disorder:0.25), (CALCIUM BICARBONATE:Sleep disorder:0.24), (CALCIUM CARBIMIDE:Sleep disorder:0.28), (CALCIUM CARBONATEERGOCALCIFEROL:Sleep disorder:0.92), (CALCIUM PHOSPHATE:Sleep disorder:0.02), (CANNABIS SATIVA SUBSP. SATIVA FLOWERING TOP:Sleep disorder:0.49), (CARBAMIDE PEROXIDE:Sleep disorder:1.3), (CARBOHYDRATESFATTY ACIDSMINERALSPROTEINVITAMINS:Sleep disorder:0.24), (CARMUSTINE:Sleep disorder:0.22), (CARTEOLOL:Sleep disorder:0.49), (CEFOTAXIMECEFOTAXIME SODIUM:Sleep disorder:0.75), (CEFTAZIDIME SODIUM:Sleep disorder:0.07), (CHAMOMILE:Sleep disorder:0.27), (CHLOPHEDIANOL HYDROCHLORIDEDEXBROMPHENIRAMINE MALEATEPHENYLEPHRINE HYDROCHLORIDE:Sleep disorder:0), (CHLORAMPHENICOL:Sleep disorder:0.62), (CHLORDIAZEPOXIDECLIDINIUM:Sleep disorder:0.17), (CHLORMADINONE ACETATE:Sleep disorder:0.1), (CHLORMADINONE ACETATEETHINYL ESTRADIOL:Sleep disorder:0.2), (CHLOROQUINE PHOSPHATE:Sleep disorder:0.5), (CHOLECALCIFEROLGENISTEINZINC GLYCINATE:Sleep disorder:0.18), (CICLOPIROX OLAMINE:Sleep disorder:0.45), (CILASTATIN SODIUMIMIPENEM:Sleep disorder:2.45), (CILAZAPRIL ANHYDROUS:Sleep disorder:0.19), (CINACALCET:Sleep disorder:0.13), (CISATRACURIUM:Sleep disorder:0.45), (CLOXACILLIN SODIUM:Sleep disorder:0.32), (COBALAMIN:Sleep disorder:0.4), (COBICISTATELVITEGRAVIREMTRICITABINETENOFOVIR:Sleep disorder:0.23), (COLCHICINEDICYCLOMINE HYDROCHLORIDE:Sleep disorder:0.03), (COLISTIN:Sleep disorder:0.61), (COPPER OR LEVONORGESTREL:Sleep disorder:0.22), (CYANOCOBALAMINPYRIDOXINE HYDROCHLORIDE\RIBOFLAVIN\SODIUM PANTOTHENATETHIAMINE HYDROCHLORIDEVITAMIN B COMPLEX:Sleep disorder:0.13), (CYCLOPENTOLATE:Sleep disorder:0.51), (CYPROTERONE:Sleep disorder:0.15), (CYPROTERONE ACETATE:Sleep disorder:0.66), (DESFLURANE:Sleep disorder:0.82), (DESLORATADINEPSEUDOEPHEDRINE SULFATE:Sleep disorder:0.16), (DEUTETRABENAZINE:Sleep disorder:0.45), (DEXTROMETHORPHANDEXTROMETHORPHAN HYDROBROMIDEGUAIFENESIN:Sleep disorder:0.07), (DIACEREIN:Sleep disorder:0.06), (DICHLORPHENAMIDE:Sleep disorder:0.08), (DICLOFENAC DIETHYLAMINE:Sleep disorder:0.24), (DIENOGEST:Sleep disorder:0.4), (DIFLUNISAL:Sleep disorder:1.37), (DIHYDRALAZINE SULFATE:Sleep disorder:0.08), (DIHYDROCODEINE BITARTRATE:Sleep disorder:0.11), (DIPHTHERIA AND TETANUS TOXOIDS AND ACELLULAR PERTUSSIS VACCINE ADSORBED:Sleep disorder:0.57), (DIPYRONEPROMETHAZINE HYDROCHLORIDE:Sleep disorder:1.97), (DISOPYRAMIDE:Sleep disorder:0.03), (DOBUTAMINE HYDROCHLORIDE:Sleep disorder:0.31), (DOCONEXENT ICOSAPENT FISH OIL OMEGA-3 FATTY ACIDS:Sleep disorder:0), (DOCUSATE SODIUMSENNOSIDE ASENNOSIDE B:Sleep disorder:0.18), (DOLASETRON MESYLATE:Sleep disorder:1.5), (DOXYCYCLINE HYDROCHLORIDE:Sleep disorder:0.58), (DROSPIRENONEESTRADIOL:Sleep disorder:0.23), (DYDROGESTERONE:Sleep disorder:0.25), (DYDROGESTERONEESTRADIOL:Sleep disorder:0.2), (EDOXABAN TOSILATE MONOHYDRATE:Sleep disorder:0.16), (EMPAGLIFLOZINLINAGLIPTIN:Sleep disorder:1.75), (EPHEDRINE HYDROCHLORIDEGUAIFENESIN:Sleep disorder:0.82), (ERIBULIN:Sleep disorder:0.94), (ERIBULIN MESYLATE:Sleep disorder:0.26), (ESCHERICHIA COLI:Sleep disorder:0.05), (ESLICARBAZEPINE ACETATE:Sleep disorder:0.09), (ESTRADIOL ACETATE:Sleep disorder:0.61), (ESTRADIOL HEMIHYDRATE:Sleep disorder:0.48), (ESTRONE:Sleep disorder:0.03), (ETHINYL ESTRADIOLFERROUS FUMARATENORETHINDRONE:Sleep disorder:0.52), (ETHINYL ESTRADIOLGESTODENE:Sleep disorder:1.2), (ETOPOSIDE PHOSPHATE:Sleep disorder:0.12), (ETRETINATE:Sleep disorder:0.23), (EUCALYPTOLMENTHOLMETHYL SALICYLATETHYMOL:Sleep disorder:0.22), (FENOPROFEN CALCIUM:Sleep disorder:0.52), (FERRIC HYDROXIDE:Sleep disorder:0.02), (FLUCYTOSINE:Sleep disorder:0.35), (FLUNARIZINE:Sleep disorder:0.04), (FLUOROURACILLEUCOVORIN CALCIUMOXALIPLATIN:Sleep disorder:0.66), (FLUPENTIXOL:Sleep disorder:0.97), (FLUPENTIXOL DECANOATE:Sleep disorder:0.37), (FLUPENTIXOL DIHYDROCHLORIDE:Sleep disorder:0.21), (FLUPHENAZINE DECANOATE:Sleep disorder:0.17), (FLUPIRTINE:Sleep disorder:0.19), (FLUTAMIDE:Sleep disorder:0.37), (FONDAPARINUX:Sleep disorder:1.23), (FORMOTEROL:Sleep disorder:2.56), (FOSFOMYCIN:Sleep disorder:0.61), (FUSIDIC ACID:Sleep disorder:2.5), (GENTIAN VIOLET:Sleep disorder:0.01), (GLATIRAMER:Sleep disorder:0.02), (GLIPIZIDEMETFORMIN HYDROCHLORIDE:Sleep disorder:1.18), (GLUCOSAMINE HYDROCHLORIDE:Sleep disorder:0.17), (GLYCOPYRROLATEINDACATEROL MALEATE:Sleep disorder:1.49), (GOLD:Sleep disorder:0.49), (GUAIFENESINPHENYLEPHRINE HYDROCHLORIDE:Sleep disorder:0.52), (HALCINONIDE:Sleep disorder:0.43), (HOMATROPINE METHYLBROMIDEHYDROCODONE BITARTRATE:Sleep disorder:0.45), (HORSE CHESTNUT:Sleep disorder:1.22), (HUMAN IMMUNOGLOBULIN GHYALURONIDASE RECOMBINANT HUMAN:Sleep disorder:0.17), (HYDRALAZINE HYDROCHLORIDEISOSORBIDE DINITRATE:Sleep disorder:0.11), (HYDROCHLORIC ACID:Sleep disorder:0.11), (HYDROCHLOROTHIAZIDEMETOPROLOL SUCCINATE:Sleep disorder:0.36), (HYDROCHLOROTHIAZIDEMETOPROLOL TARTRATE:Sleep disorder:0.5), (HYDROCORTISONENEOMYCIN SULFATEPOLYMYXIN B SULFATE:Sleep disorder:0.23), (HYDROQUINONE:Sleep disorder:0.05), (HYDROXYPROPYL CELLULOSE:Sleep disorder:0.6), (IDARUBICIN:Sleep disorder:0.19), (IMIPENEM:Sleep disorder:0.81), (IMIPRAMINE PAMOATE:Sleep disorder:0.3), (INSULIN PORKINSULIN PURIFIED PORK:Sleep disorder:0.92), (INTERFERON ALFA-2A OR INTERFERON ALFA-2B:Sleep disorder:0.11), (INTERFERON GAMMA-1B:Sleep disorder:1.58), (IODINE\POTASSIUM IODIDE:Sleep disorder:0.36), (ISAVUCONAZONIUM SULFATE:Sleep disorder:0.24), (ISOFLAVONES SOY:Sleep disorder:0.06), (ISONIAZIDRIFAMPIN:Sleep disorder:0.76), (ISOPROPYL ALCOHOLLEUPROLIDE ACETATENORETHINDRONE ACETATE:Sleep disorder:0.48), (IVACAFTOR:Sleep disorder:0.56), (LANREOTIDE:Sleep disorder:1.31), (LARONIDASE:Sleep disorder:0.79), (LATANOPROSTTIMOLOL:Sleep disorder:0.79), (LEVAMLODIPINE:Sleep disorder:0.15), (LEVOBUNOLOL:Sleep disorder:0.38), (LEVOMEFOLIC ACIDMETHYLCOBALAMINTURMERIC:Sleep disorder:0.4), (LIMAPROST:Sleep disorder:0.03), (LINSIDOMINE:Sleep disorder:0.39), (LITHIUM SULFATE:Sleep disorder:0.18), (LOFEPRAMINE:Sleep disorder:0.1), (LOMUSTINE:Sleep disorder:0.11), (LOVASTATINNIACIN:Sleep disorder:0.23), (LURASIDONE:Sleep disorder:0.46), (LUTETIUM LU-177:Sleep disorder:1.02), (LYCOPENE:Sleep disorder:0.3), (MAGNESIUM SULFATE\POTASSIUM SULFATE\SODIUM SULFATE:Sleep disorder:0.28), (MANGANESEMANGANESE CHLORIDE:Sleep disorder:0.29), (MANNITOL:Sleep disorder:0.42), (MATRICARIA RECUTITA:Sleep disorder:0.52), (MENTHOLZINC OXIDE:Sleep disorder:0.86), (MESTRANOLNORETHINDRONE:Sleep disorder:0.47), (METFORMIN HYDROCHLORIDEROSIGLITAZONE MALEATE:Sleep disorder:0.29), (METFORMIN HYDROCHLORIDESAXAGLIPTIN:Sleep disorder:0.48), (METHAMPHETAMINE HYDROCHLORIDE:Sleep disorder:0.06), (METHENAMINE MANDELATE:Sleep disorder:1.35), (METHENOLONE ENANTHATE:Sleep disorder:0.1), (METHOXY POLYETHYLENE GLYCOL-EPOETIN BETA:Sleep disorder:0.08), (METHSUXIMIDE:Sleep disorder:0.39), (METILDIGOXIN:Sleep disorder:0.08), (METRELEPTIN:Sleep disorder:0.97), (METYROSINE:Sleep disorder:0.2), (MIGLITOL:Sleep disorder:0.37), (MINERAL OIL:Sleep disorder:0.08), (MIZORIBINE:Sleep disorder:0.16), (MOEXIPRIL HYDROCHLORIDE:Sleep disorder:1.4), (MORNIFLUMATE:Sleep disorder:0.37), (MORPHINE\MORPHINE HYDROCHLORIDE\MORPHINE SULFATE\MORPHINE TARTRATE:Sleep disorder:0.44), (NALOXONE HYDROCHLORIDEPENTAZOCINE HYDROCHLORIDE:Sleep disorder:0.09), (NEOSTIGMINE:Sleep disorder:1.37), (NOREPINEPHRINE:Sleep disorder:1.22), (NUSINERSEN:Sleep disorder:0.81), (OLODATEROL HYDROCHLORIDE:Sleep disorder:0.53), (OMEGA-6 FATTY ACIDS:Sleep disorder:0.44), (PAPAYA:Sleep disorder:0.65), (PARSLEY:Sleep disorder:0.14), (PASSIFLORA INCARNATA FLOWER:Sleep disorder:0.12), (PEGINTERFERON ALFA-2A OR PEGINTERFERON ALFA-2B:Sleep disorder:0.12), (PENICILLAMINE:Sleep disorder:0.48), (PENICILLIN V BENZATHINE:Sleep disorder:0.18), (PENTAERYTHRITOL TETRANITRATE:Sleep disorder:0.08), (PENTOSTATIN:Sleep disorder:0.73), (PEPSIN:Sleep disorder:0.27), (PERAMPANEL:Sleep disorder:1.22), (PHENELZINE:Sleep disorder:0.73), (PHENYLEPHRINE HYDROCHLORIDEWITCH HAZEL:Sleep disorder:0.07), (PIRIBEDIL:Sleep disorder:0.07), (PIRITRAMIDE:Sleep disorder:0.65), (PIZOTYLINE:Sleep disorder:0.91), (POLYETHYLENE GLYCOL 400:Sleep disorder:0.79), (POLYMYXIN:Sleep disorder:0.09), (PORFIMER SODIUM:Sleep disorder:0.02), (POVIDONE:Sleep disorder:0.64), (PRANLUKAST:Sleep disorder:0.34), (PRISTINAMYCIN:Sleep disorder:0.63), (PROCYCLIDINE:Sleep disorder:0.8), (PROPANTHELINE BROMIDE:Sleep disorder:0.09), (PROPATYL NITRATE:Sleep disorder:0.87), (PRUCALOPRIDE:Sleep disorder:0.07), (PSORALEN:Sleep disorder:0.11), (PYRAZINAMIDE:Sleep disorder:0.8), (PYRIMETHAMINE:Sleep disorder:0.73), (QUINIDINE GLUCONATE:Sleep disorder:0.18), (RAMUCIRUMAB:Sleep disorder:1.16), (REBOXETINE:Sleep disorder:0.12), (REMIFENTANIL HYDROCHLORIDE:Sleep disorder:0.1), (RESLIZUMAB:Sleep disorder:0.08), (RILMENIDINE PHOSPHATE:Sleep disorder:1.13), (ROFECOXIB:Sleep disorder:1.3), (ROPIVACAINE HYDROCHLORIDE:Sleep disorder:0.2), (RUPATADINE FUMARATE:Sleep disorder:0.24), (SAFINAMIDE MESYLATE:Sleep disorder:0.28), (SARPOGRELATE:Sleep disorder:0.28), (SERRAPEPTASE:Sleep disorder:0.37), (SERTINDOLE:Sleep disorder:0.56), (SODIUM CHONDROITIN SULFATE:Sleep disorder:0.09), (SODIUM CITRATE:Sleep disorder:0.56), (SODIUM FLUORIDEVITAMINS:Sleep disorder:0.45), (SODIUM HYPOCHLORITE:Sleep disorder:0.07), (SOLIFENACIN:Sleep disorder:0.55), (SOMATREM:Sleep disorder:0.11), (SUCCINYLCHOLINE CHLORIDE:Sleep disorder:0.58), (SULTOPRIDE HYDROCHLORIDE:Sleep disorder:0.42), (SUNITINIB:Sleep disorder:0.1), (TALIGLUCERASE ALFA:Sleep disorder:0.27), (TAZAROTENE:Sleep disorder:0.29), (TBO-FILGRASTIM:Sleep disorder:0.02), (TELOTRISTAT ETHYL:Sleep disorder:1.18), (TENOXICAM:Sleep disorder:0.38), (TERBUTALINE:Sleep disorder:1.92), (TESTOSTERONE UNDECANOATE:Sleep disorder:2.21), (THIOPENTAL SODIUM:Sleep disorder:0.12), (THIORIDAZINE:Sleep disorder:0.21), (TICLOPIDINE:Sleep disorder:0.52), (TIOPRONIN:Sleep disorder:0.28), (TOLMETIN SODIUM:Sleep disorder:0.2), (TREPROSTINIL DIOLAMINE:Sleep disorder:0.8), (TRIPTORELIN PAMOATE:Sleep disorder:1.38), (VALDECOXIB:Sleep disorder:1.18), (VASOPRESSIN:Sleep disorder:0.42), (VELAGLUCERASE ALFA:Sleep disorder:0.32), (VITAMIN A PALMITATE:Sleep disorder:0.41), (VOGLIBOSE:Sleep disorder:0.18), (WARFARIN POTASSIUM:Sleep disorder:0.3), (ZEAXANTHIN:Sleep disorder:0.58), (ZINC ACETATE:Sleep disorder:0.45), (ZIV-AFLIBERCEPT:Sleep disorder:0.64), (ZUCLOPENTHIXOL DECANOATE:Sleep disorder:0.52), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Sleep disorder therapy:0.04), (ACARBOSE:Sleep disorder therapy:0.18), (ACETAMINOPHENOXYCODONE HYDROCHLORIDEOXYCODONE TEREPHTHALATE:Sleep disorder therapy:0.69), (AGALSIDASE BETA:Sleep disorder therapy:0.04), (ALBIGLUTIDE:Sleep disorder therapy:0.17), (ALEMTUZUMAB:Sleep disorder therapy:0.03), (ALUMINUM HYDROXIDEDIMETHICONEMAGNESIUM HYDROXIDE:Sleep disorder therapy:0.07), (AMISULPRIDE:Sleep disorder therapy:0.77), (AMLODIPINE BESYLATEHYDROCHLOROTHIAZIDEVALSARTAN:Sleep disorder therapy:0.12), (AMLODIPINEVALSARTAN:Sleep disorder therapy:0.12), (ARIPIPRAZOLE LAUROXIL:Sleep disorder therapy:0.3), (ARMODAFINIL:Sleep disorder therapy:1.77), (AZITHROMYCIN DIHYDRATE:Sleep disorder therapy:0.67), (BETAMETHASONE DIPROPIONATECALCIPOTRIENECALCIPOTRIENE HYDRATE:Sleep disorder therapy:0.08), (BISACODYL:Sleep disorder therapy:0.69), (BRINZOLAMIDE:Sleep disorder therapy:0.62), (BUPRENORPHINE HYDROCHLORIDE:Sleep disorder therapy:0.83), (CALCITONIN SALMON:Sleep disorder therapy:0.25), (CALCIUM ACETATE:Sleep disorder therapy:0.55), (CALCIUM\MAGNESIUM:Sleep disorder therapy:0.11), (CANAKINUMAB:Sleep disorder therapy:0.24), (CANDESARTAN CILEXETILHYDROCHLOROTHIAZIDE:Sleep disorder therapy:1.1), (CARVEDILOL PHOSPHATE:Sleep disorder therapy:1.26), (CEFTRIAXONE SODIUM:Sleep disorder therapy:0.32), (CHONDROITIN SULFATE (BOVINE)GLUCOSAMINE HYDROCHLORIDE:Sleep disorder therapy:0.61), (CICLESONIDE:Sleep disorder therapy:0.15), (CLARITHROMYCIN:Sleep disorder therapy:0.14), (CLINDAMYCIN:Sleep disorder therapy:0.77), (CORTICOTROPIN:Sleep disorder therapy:0.49), (DABIGATRAN:Sleep disorder therapy:0.27), (DACLIZUMAB:Sleep disorder therapy:0.55), (DANAZOL:Sleep disorder therapy:0.3), (DASATINIB:Sleep disorder therapy:1.42), (DICLOFENAC SODIUMMISOPROSTOL:Sleep disorder therapy:0.01), (DOXEPIN HYDROCHLORIDE:Sleep disorder therapy:0.76), (DRONEDARONE:Sleep disorder therapy:0.21), (EMPAGLIFLOZINLINAGLIPTIN:Sleep disorder therapy:0.45), (EVEROLIMUS:Sleep disorder therapy:0.96), (EVOLOCUMAB:Sleep disorder therapy:1.48), (FEBUXOSTAT:Sleep disorder therapy:0.42), (FENOFIBRIC ACID:Sleep disorder therapy:0.35), (FESOTERODINE FUMARATE:Sleep disorder therapy:0.99), (FINGOLIMOD HYDROCHLORIDE:Sleep disorder therapy:0.47), (FISH OIL\OMEGA-3 FATTY ACIDS:Sleep disorder therapy:0.53), (FLUTICASONE FUROATEVILANTEROL TRIFENATATE:Sleep disorder therapy:0.93), (FLUVOXAMINE MALEATE:Sleep disorder therapy:0.4), (FUSIDIC ACID:Sleep disorder therapy:0.2), (GLYCOPYRROLATE:Sleep disorder therapy:0.06), (GLYCOPYRRONIUMINDACATEROL:Sleep disorder therapy:0.49), (HYDROCHLOROTHIAZIDEIRBESARTAN:Sleep disorder therapy:0.9), (HYDROCODONE BITARTRATEIBUPROFEN:Sleep disorder therapy:0.21), (HYDROXYCHLOROQUINE DIPHOSPHATE:Sleep disorder therapy:0.63), (ILOPROST:Sleep disorder therapy:0.71), (IMATINIB MESYLATE:Sleep disorder therapy:0.93), (IMIPRAMINE HYDROCHLORIDE:Sleep disorder therapy:0.71), (INDAPAMIDEPERINDOPRIL:Sleep disorder therapy:0.36), (INFLIXIMAB-DYYB:Sleep disorder therapy:0.18), (INSULIN DEGLUDEC:Sleep disorder therapy:0.19), (IODINE:Sleep disorder therapy:0.02), (LOMITAPIDE MESYLATE:Sleep disorder therapy:0.56), (METFORMIN HYDROCHLORIDEVILDAGLIPTIN:Sleep disorder therapy:0.02), (METHENAMINE HIPPURATE:Sleep disorder therapy:0.9), (METHYLPREDNISOLONE ACETATE:Sleep disorder therapy:1.56), (MIDODRINE HYDROCHLORIDE:Sleep disorder therapy:0.05), (MUPIROCINMUPIROCIN CALCIUM:Sleep disorder therapy:0.48), (NALTREXONE:Sleep disorder therapy:0.41), (NILOTINIB:Sleep disorder therapy:0.91), (OXYMORPHONE:Sleep disorder therapy:0.14), (PATIROMER:Sleep disorder therapy:0.03), (PAZOPANIB HYDROCHLORIDE:Sleep disorder therapy:0.55), (PEGINTERFERON BETA-1A:Sleep disorder therapy:0.89), (PENICILLIN:Sleep disorder therapy:0.99), (PIROXICAM:Sleep disorder therapy:0.35), (PONATINIB:Sleep disorder therapy:0.79), (RIVASTIGMINE:Sleep disorder therapy:0.6), (RIZATRIPTAN:Sleep disorder therapy:0.16), (ROFLUMILAST:Sleep disorder therapy:0.68), (SALMETEROL:Sleep disorder therapy:0.06), (SALMETEROL XINAFOATE:Sleep disorder therapy:0.14), (SECUKINUMAB:Sleep disorder therapy:0.73), (SELEGILINE:Sleep disorder therapy:0.03), (SITAGLIPTIN:Sleep disorder therapy:0.1), (SODIUM OXYBATE:Sleep disorder therapy:1.95), (SULFAMETHOXAZOLE:Sleep disorder therapy:0.54), (TERBUTALINE SULFATE:Sleep disorder therapy:0.68), (TERIFLUNOMIDE:Sleep disorder therapy:0.47), (TETRABENAZINE:Sleep disorder therapy:0.1), (TETRACYCLINE:Sleep disorder therapy:1.05), (THALIDOMIDE:Sleep disorder therapy:0.29), (TIMOLOL:Sleep disorder therapy:1.24), (TOBRAMYCIN:Sleep disorder therapy:0.01), (TOLTERODINE:Sleep disorder therapy:0.01), (UMECLIDINIUM BROMIDE:Sleep disorder therapy:0.12), (ZAFIRLUKAST:Sleep disorder therapy:0.13), (ZOLMITRIPTAN:Sleep disorder therapy:0.13), (ACETAZOLAMIDE:Small cell lung cancer:0.27), (ADO-TRASTUZUMAB EMTANSINE:Small cell lung cancer:0.34), (ALFACALCIDOL:Small cell lung cancer:0), (AMIODARONE HYDROCHLORIDE:Small cell lung cancer:1), (ANASTROZOLE:Small cell lung cancer:0.83), (BORTEZOMIB:Small cell lung cancer:0.02), (BUPROPION:Small cell lung cancer:0.1), (BUSPIRONE HYDROCHLORIDE:Small cell lung cancer:0.06), (CALCIUM ACETATE:Small cell lung cancer:0.12), (CARVEDILOL PHOSPHATE:Small cell lung cancer:0.1), (CEFOTAXIME SODIUM:Small cell lung cancer:0.18), (CERTOLIZUMAB PEGOL:Small cell lung cancer:0.54), (CETUXIMAB:Small cell lung cancer:0.64), (CILASTATIN SODIUMIMIPENEM:Small cell lung cancer:0.15), (CIMETIDINE:Small cell lung cancer:0.35), (CIPROFLOXACIN HYDROCHLORIDE:Small cell lung cancer:0.14), (CLONIDINE:Small cell lung cancer:0.1), (CLOTRIMAZOLE:Small cell lung cancer:0.2), (CORTISONEHYDROCORTISONE:Small cell lung cancer:0.61), (CYCLOBENZAPRINE:Small cell lung cancer:0.15), (DABIGATRAN:Small cell lung cancer:0.05), (DABRAFENIB:Small cell lung cancer:0.01), (DASATINIB:Small cell lung cancer:0.76), (DULOXETINE:Small cell lung cancer:0.15), (EPINEPHRINE:Small cell lung cancer:0.47), (EPIRUBICIN:Small cell lung cancer:0.75), (EPLERENONE:Small cell lung cancer:0.09), (EVOLOCUMAB:Small cell lung cancer:0.25), (EXEMESTANE:Small cell lung cancer:0), (FEBUXOSTAT:Small cell lung cancer:0.33), (HYDROCHLOROTHIAZIDELOSARTAN POTASSIUM:Small cell lung cancer:0.13), (HYDROCHLOROTHIAZIDEVALSARTAN:Small cell lung cancer:0.08), (IBRUTINIB:Small cell lung cancer:0.08), (INDAPAMIDEPERINDOPRIL:Small cell lung cancer:0.57), (INFLUENZA VIRUS VACCINE:Small cell lung cancer:0.18), (INSULIN DETEMIR:Small cell lung cancer:0.1), (ISOSORBIDE:Small cell lung cancer:0.26), (LABETALOLLABETALOL HYDROCHLORIDE:Small cell lung cancer:0.19), (LATANOPROST:Small cell lung cancer:0.26), (LEDIPASVIRSOFOSBUVIR:Small cell lung cancer:0.37), (LERCANIDIPINE:Small cell lung cancer:0.11), (LERCANIDIPINE HYDROCHLORIDE:Small cell lung cancer:0.22), (LEUCOVORIN CALCIUM:Small cell lung cancer:0.93), (METHADONE HYDROCHLORIDE:Small cell lung cancer:0.16), (METOLAZONE:Small cell lung cancer:0.1), (NILOTINIB:Small cell lung cancer:1.01), (OXCARBAZEPINE:Small cell lung cancer:0.21), (PANITUMUMAB:Small cell lung cancer:0.18), (PANTOPRAZOLE MAGNESIUM:Small cell lung cancer:0.64), (PAROXETINEPAROXETINE HYDROCHLORIDE:Small cell lung cancer:0.12), (PENICILLIN:Small cell lung cancer:0.29), (PITAVASTATIN CALCIUM:Small cell lung cancer:0.08), (PONATINIB:Small cell lung cancer:0.02), (PRIMIDONE:Small cell lung cancer:0.26), (QUETIAPINE:Small cell lung cancer:0.05), (RABEPRAZOLE:Small cell lung cancer:0.04), (RANOLAZINE:Small cell lung cancer:0.36), (REGORAFENIB:Small cell lung cancer:0.52), (RIFAMPIN:Small cell lung cancer:0.74), (RISEDRONATE SODIUM:Small cell lung cancer:0.27), (SECUKINUMAB:Small cell lung cancer:0.07), (SOTALOL:Small cell lung cancer:0.55), (TERIFLUNOMIDE:Small cell lung cancer:0.06), (TETRACYCLINE:Small cell lung cancer:0.23), (THALIDOMIDE:Small cell lung cancer:0.32), (TOPIRAMATE:Small cell lung cancer:0.71), (TRAMETINIB:Small cell lung cancer:0.07), (VANCOMYCIN HYDROCHLORIDE:Small cell lung cancer:0.37), (VINORELBINE TARTRATE:Small cell lung cancer:0.16), (.ALPHA.1-PROTEINASE INHIBITOR HUMAN:Smoking cessation therapy:0.34), (ACETAMINOPHENPROPOXYPHENE HYDROCHLORIDE:Smoking cessation therapy:0.57), (AGALSIDASE BETA:Smoking cessation therapy:0.13), (ALEMTUZUMAB:Smoking cessation therapy:0.03), (ALENDRONIC ACID:Smoking cessation therapy:0.54), (ALISKIREN HEMIFUMARATE:Smoking cessation therapy:0.54), (AMLODIPINE BESYLATEHYDROCHLOROTHIAZIDEOLMESARTAN MEDOXOMIL:Smoking cessation therapy:0.47), (AMOXICILLINCLAVULANIC ACID:Smoking cessation therapy:0.19), (ANTIHISTAMINES NOS:Smoking cessation therapy:0.24), (ARIPIPRAZOLE LAUROXIL:Smoking cessation therapy:0.53), (BETAMETHASONE DIPROPIONATECALCIPOTRIENECALCIPOTRIENE HYDRATE:Smoking cessation therapy:0.36), (BETAMETHASONECALCIPOTRIENE:Smoking cessation therapy:0.12), (BRINZOLAMIDE:Smoking cessation therapy:0.45), (BUTYLSCOPOLAMINE BROMIDE:Smoking cessation therapy:0.27), (CALCITONIN SALMON:Smoking cessation therapy:0.35), (CALCIUM\MAGNESIUM:Smoking cessation therapy:0.36), (CALCIUM\MAGNESIUM\ZINC:Smoking cessation therapy:0.29), (CARVEDILOL PHOSPHATE:Smoking cessation therapy:1.21), (CEVIMELINE HYDROCHLORIDE:Smoking cessation therapy:0.03), (CILASTATIN SODIUMIMIPENEM:Smoking cessation therapy:0.01), (CLOMIPRAMINE HYDROCHLORIDE:Smoking cessation therapy:0.24), (CODEINE PHOSPHATEGUAIFENESIN:Smoking cessation therapy:0.03), (CYCLOPHOSPHAMIDE:Smoking cessation therapy:0.07), (DACLIZUMAB:Smoking cessation therapy:0.97), (DALTEPARIN:Smoking cessation therapy:0.49), (DANAZOL:Smoking cessation therapy:0.32), (DAPAGLIFLOZIN PROPANEDIOL:Smoking cessation therapy:1.03), (DARBEPOETIN ALFA:Smoking cessation therapy:0.23), (DEFLAZACORT:Smoking cessation therapy:0.48), (DEVICE:Smoking cessation therapy:0.47), (DEXTROAMPHETAMINE:Smoking cessation therapy:0.03), (DIPYRIDAMOLE:Smoking cessation therapy:0.17), (DIPYRONE:Smoking cessation therapy:0.42), (DOFETILIDE:Smoking cessation therapy:0.73), (DOXYLAMINE SUCCINATE:Smoking cessation therapy:0.07), (DRONEDARONE:Smoking cessation therapy:0.22), (DUTASTERIDETAMSULOSIN HYDROCHLORIDE:Smoking cessation therapy:0.34), (ECONAZOLE:Smoking cessation therapy:0.18), (EMPAGLIFLOZINLINAGLIPTIN:Smoking cessation therapy:0.5), (EPLERENONE:Smoking cessation therapy:0.7), (ESTROGENS:Smoking cessation therapy:0.22), (EVOLOCUMAB:Smoking cessation therapy:2.01), (FORMOTEROL:Smoking cessation therapy:0.53), (FUSIDIC ACID:Smoking cessation therapy:0.68), (GUANFACINE HYDROCHLORIDE:Smoking cessation therapy:0.94), (HYALURONATE SODIUM:Smoking cessation therapy:0.34), (HYDROCHLOROTHIAZIDETELMISARTAN:Smoking cessation therapy:0.55), (HYDROCODONE BITARTRATEIBUPROFEN:Smoking cessation therapy:0.37), (IBRUTINIB:Smoking cessation therapy:0.61), (ILOPROST:Smoking cessation therapy:0.2), (IMIPRAMINE HYDROCHLORIDE:Smoking cessation therapy:1), (INTERFERON:Smoking cessation therapy:0.07), (KETOROLACKETOROLAC TROMETHAMINE:Smoking cessation therapy:0.37), (LABETALOL HYDROCHLORIDE:Smoking cessation therapy:0.13), (LERCANIDIPINE:Smoking cessation therapy:0.95), (LERCANIDIPINE HYDROCHLORIDE:Smoking cessation therapy:0.67), (LEVALBUTEROL:Smoking cessation therapy:0.04), (LEVOMEFOLATE CALCIUM:Smoking cessation therapy:0.51), (LEVOMEFOLATE CALCIUM\METHYLCOBALAMINPYRIDOXAL PHOSPHATE ANHYDROUS:Smoking cessation therapy:0.05), (LIDOCAINE HYDROCHLORIDE:Smoking cessation therapy:1.28), (LIDOCAINEPRILOCAINE:Smoking cessation therapy:0.74), (LIOTHYRONINE:Smoking cessation therapy:0.22), (MEPOLIZUMAB:Smoking cessation therapy:0.81), (MEROPENEM:Smoking cessation therapy:0.02), (METFORMIN HYDROCHLORIDEVILDAGLIPTIN:Smoking cessation therapy:0.06), (MIDAZOLAMMIDAZOLAM HYDROCHLORIDE:Smoking cessation therapy:0.05), (MIDODRINE HYDROCHLORIDE:Smoking cessation therapy:0.36), (MOEXIPRIL HYDROCHLORIDE:Smoking cessation therapy:0.08), (MOXIFLOXACIN:Smoking cessation therapy:0.18), (MOXIFLOXACIN HYDROCHLORIDE:Smoking cessation therapy:1.92), (NALOXONE HYDROCHLORIDEOXYCODONE HYDROCHLORIDE:Smoking cessation therapy:1.11), (NATEGLINIDE:Smoking cessation therapy:0.29), (NILOTINIB:Smoking cessation therapy:1.48), (NIRAPARIB:Smoking cessation therapy:0.01), (NORTRIPTYLINE HYDROCHLORIDE:Smoking cessation therapy:0.69), (OSELTAMIVIR PHOSPHATE:Smoking cessation therapy:0.52), (OXYBUTYNINOXYBUTYNIN CHLORIDE:Smoking cessation therapy:0.05), (PANCRELIPASE:Smoking cessation therapy:0.03), (PATIROMER:Smoking cessation therapy:0), (PAZOPANIB HYDROCHLORIDE:Smoking cessation therapy:1.02), (PEGINTERFERON BETA-1A:Smoking cessation therapy:1), (PENTOSAN POLYSULFATE SODIUM:Smoking cessation therapy:0.62), (PIMECROLIMUS:Smoking cessation therapy:0.42), (PIRFENIDONE:Smoking cessation therapy:0.02), (PONATINIB:Smoking cessation therapy:0.73), (PREDNISOLONE ACETATE:Smoking cessation therapy:0.54), (PREDNISOLONEPREDNISOLONE ACETATEPREDNISOLONE SODIUM SUCCINATE:Smoking cessation therapy:0.51), (PROPAFENONE HYDROCHLORIDE:Smoking cessation therapy:0.49), (PROTRIPTYLINE HYDROCHLORIDE:Smoking cessation therapy:0.15), (RALOXIFENE HYDROCHLORIDE:Smoking cessation therapy:0.89), (RETINOL:Smoking cessation therapy:0.27), (RIVASTIGMINE TARTRATE:Smoking cessation therapy:0.46), (RIZATRIPTAN:Smoking cessation therapy:0.24), (SACCHAROMYCES CEREVISIAE:Smoking cessation therapy:0.15), (SACUBITRILVALSARTAN:Smoking cessation therapy:0.5), (SELEGILINE:Smoking cessation therapy:0.09), (SODIUM FLUORIDE:Smoking cessation therapy:0.07), (SOFOSBUVIRVELPATASVIR:Smoking cessation therapy:0.02), (TESTOSTERONE UNDECANOATE:Smoking cessation therapy:0.25), (TETRACYCLINE:Smoking cessation therapy:1.37), (VALGANCICLOVIR HYDROCHLORIDE:Smoking cessation therapy:0.22), (VARDENAFIL HYDROCHLORIDE TRIHYDRATE:Smoking cessation therapy:0.04), (ZILEUTON:Smoking cessation therapy:0.1), (EPINEPHRINE:Sneezing:0.07), (EVOLOCUMAB:Sneezing:0.26), (IBRUTINIB:S neezing:0.12), (NAPROXEN:Sneezing:0.29), (OXYGEN:Sneezing:0.27), (TERIFLUNOMIDE:Sn eezing:0.13), (ADO-TRASTUZUMAB EMTANSINE:Soft tissue sarcoma:0.16), (AMIODARONE HYDROCHLORIDE:Soft tissue sarcoma:0.16), (ANASTROZOLE:Soft tissue sarcoma:0.83), (BUDESONIDE:Soft tissue sarcoma:0.37), (CANDESARTAN:Soft tissue sarcoma:0.13), (CANDESARTAN CILEXETIL:Soft tissue sarcoma:0.1), (COLCHICINE:Soft tissue sarcoma:0.13), (CYCLOBENZAPRINE:Soft tissue sarcoma:0.01), (DEXLANSOPRAZOLE:Soft tissue sarcoma:0.96), (DILTIAZEM HYDROCHLORIDE:Soft tissue sarcoma:0.43), (DIVALPROEX SODIUM:Soft tissue sarcoma:0.2), (EPINEPHRINE:Soft tissue sarcoma:0.22), (EVOLOCUMAB:Soft tissue sarcoma:0.07), (EXEMESTANE:Soft tissue sarcoma:0.21), (METRONIDAZOLE:Soft tissue sarcoma:0.57), (MONTELUKAST SODIUM:Soft tissue sarcoma:0.3), (NAPROXEN:Soft tissue sarcoma:0.48), (NEBIVOLOL:Soft tissue sarcoma:0.16), (NITROGLYCERIN:Soft tissue sarcoma:0.11), (PHENOBARBITAL:Soft tissue sarcoma:0.11), (POTASSIUM:Soft tissue sarcoma:0.56), (PRAVASTATIN SODIUM:Soft tissue sarcoma:0.2), (PRIMIDONE:Soft tissue sarcoma:0.16), (RANOLAZINE:Soft tissue sarcoma:0.16), (SACUBITRILVALSARTAN:Soft tissue sarcoma:0.21), (THALIDOMIDE:Soft tissue sarcoma:0.44), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Somnolence:0.67), (.ALPHA.1-PROTEINASE INHIBITOR HUMAN:Somnolence:0.41), (ACETAMINOPHENPROPOXYPHENE HYDROCHLORIDE:Somnolence:0.33), (ALBIGLUTIDE:Somnolence:0.28), (ALENDRONIC ACID:Somnolence:0.31), (ALFACALCIDOL:Somnolence:0.2), (ALISKIREN HEMIFUMARATE:Somnolence:0.46), (AMANTADINE HYDROCHLORIDE:Somnolence:0.61), (AMINO ACIDSDEXTROSEELECTROLYTES NOS:Somnolence:0.1), (AMIODARONE:Somnolence:0.78), (AMISULPRIDE:Somnolence:0.43), (AMLODIPINE BESYLATEOLMESARTAN MEDOXOMIL:Somnolence:0.08), (AMLODIPINEVALSARTAN:Somnolence:0.11), (AMMO NIUM LACTATE:Somnolence:0.54), (ANTIHISTAMINES NOS:Somnolence:0.23), (ARIPIPRAZOLE LAUROXIL:Somnolence:0.61), (BENZTROPINE:Somnolence:0.41), (BETAMETHASONE DIPROPIONATECALCIPOTRIENECALCIPOTRIENE HYDRATE:Somnolence:0.58), (BETAMETHASONECALCIPOTRIENE:Somnolence:0.17), (B RINZOLAMIDE:Somnolence:0.64), (BUDESONIDEFORMOTEROL FUMARATE:Somnolence:0.55), (BUPRENORPHINENALOXONE:Somnolence:0.57), (CALCI TONIN:Somnolence:0.01), (CALCITONIN SALMON:Somnolence:0.63), (CANAGLIFLOZINMETFORMIN HYDROCHLORIDE:Somnolence:0.67), (CANAKINUMAB:Somnolence:0.37), (CANDESART AN:Somnolence:0.69), (CANDESARTAN CILEXETILHYDROCHLOROTHIAZIDE:Somnolence:0.57), (CAPTOPRIL:Somnolence:0.22), (CEFTRIAXONE SODIUM:Somnolence:0.6), (CHLORPHENIRAMINEHYDROCODONE:Somnolence:0.31), (C HLORPROMAZINE:Somnolence:0.33), (CILOSTAZOL:Somnolence:0.13), (CODEINE PHOSPHATE:Somnolence:0.1), (COLLAGEN:Somnolence:0.06), (DACLIZUMAB:Somnolence: 1.41), (DASATINIB:Somnolence:1.07), (DEFLAZACORT:Somnolence:0.74), (DEVICE:Somnol ence:0.66), (DOFETILIDE:Somnolence:0.59), (DORZOLAMIDE:Somnolence:0.11), (DORZOL AMIDE HYDROCHLORIDE:Somnolence:0.03), (EMPAGLIFLOZINLINAGLIPTIN:Somnolence:0.49), (ENOXAPARIN:Somnolence:0.64), (EPLERENONE:Somnolence:0.08), (ESOMEPRAZOLE:S omnolence:0.6), (ESTRADIOL BENZOATE:Somnolence:0.39), (ESTRADIOL HEMIHYDRATE:Somnolence:0.17), (ETORICOXIB:Somnolence:0.37), (EVOLOCUMAB:Som nolence:1.79), (FLECAINIDE:Somnolence:0.77), (FLURAZEPAMFLURAZEPAM HYDROCHLORIDE:Somnolence:0.2), (FORMOTEROL FUMARATE\MOMETASONE FUROATE:Somnolence:0.49), (FUSIDIC ACID:Somnolence:0.12), (GLIPIZIDEMETFORMIN HYDROCHLORIDE:Somnolence:0.11), (GLYCOPYRRONIUMINDACATEROL:Somnolence: 0.2), (HERBALSTURMERIC:Somnolence:1.13), (HYDROXYUREA:Somnolence:0.58), (HYO SCYAMINE SULFATEMETHENAMINEMETHYLENE BLUEPHENYL SALICYLATE\SODIUM PHOSPHATE, MONOBASIC, MONOHYDRATE:Somnolence:0.36), (INSULIN DEGLUDEC:Somnolence:0.29), (INSULIN GLULISINE:Somnolence:0.27), (IODINE:Somnolence:0.11), (ISOPROPYL ALCOHOLLEUPROLIDE ACETATENORETHINDRONE ACETATE:Somnolence:0.24), (LABETALOLLABETALOL HYDROCHLORIDE:Somnolence:1.34), (LACTOBACILLUS RHAMNOSUS:Somnolence:0.54), (LIDOCAINEPRILOCAINE:Somnolence:0.56), (LOMITAP IDE MESYLATE:Somnolence:0.79), (MEPOLIZUMAB:Somnolence:0.28), (MICONAZOLE NITRATE:Somnolence:0.24), (NALOXONE HYDROCHLORIDEOXYCODONE HYDROCHLORIDE:Somnolence:0.5), (NILOTINIB:Somnolence:1.05), (NINTEDANIB:Somno lence:0.08), (PANTOPRAZOLE MAGNESIUM:Somnolence:1.68), (PAPAYA:Somnolence:0.14), (PATIROMER:Somnolence:0.1), (PAZOPANIB HYDROCHLORIDE:Somnolence:0.35), (PEGINTERFERON BETA-1A:Somnolence:1.14), (PENICILLIN:Somnolence:1.72), (PERPHENAZINE:Somnolence:0.02), (PHENTERMINE HYDROCHLORIDETOPIRAMATE:Somnolence:0.11), (PHENYTOIN SODIUM:Somnolence:0.27), (PONATINIB:Somnolence:0.55), (PREDNISOLONE SODIUM PHOSPHATE:Somnolence:0.21), (PREGNENOLONE:Somnolence:0.28), (PROPAFENONE HYDROCHLORIDE:Somnolence:0.18), (PROTRIPTYLINE HYDROCHLORIDE:Somnolence:1.25), (QUINIDINE GLUCONATE:Somnolence:0.15), (RABEPRAZOLE:Somnolence:0.74), (RETINOL:Somnolenc e:0.16), (ROSUVASTATIN:Somnolence:0.4), (SALMETEROL XINAFOATE:Somnolence:0.47), (SIBUTRAMINE HYDROCHLORIDE:Somnolence:0.13), (SITAGLIPTIN:Somnolence:0), (SULINDAC:Somnole nce:0.31), (TASIMELTEON:Somnolence:0.49), (TERAZOSIN HYDROCHLORIDE:Somnolence:0.17), (TERBUTALINE SULFATE:Somnolence:0.64), (TETRABENAZINE:Somnolence:0.28), (THALIDOMIDE:Somn olence:1.03), (TIOTROPIUM:Somnolence:0.13), (UMECLIDINIUM BROMIDEVILANTEROL TRIFENATATE:Somnolence:1.57), (VALGANCICLOVIR HYDROCHLORIDE:Somnolence:0.08), (VANCOMYCIN:Somnolence:0.16), (YEAST:Somnole nce:0.57), (CISATRACURIUM:Spinal anaesthesia:0.05), (ESMOLOL HYDROCHLORIDE:Spinal anaesthesia:0.08), (SUGAMMADEX:Spinal anaesthesia:0.58), (ACARBOSE:Spinal column stenosis:0.13), (ACETAZOLAMIDE:Spinal column stenosis:0.29), (ACLIDINIUM BROMIDE:Spinal column stenosis:0.06), (ALBIGLUTIDE:Spinal column stenosis:0.12), (ALENDRONIC ACID:Spinal column stenosis:0.04), (ALFACALCIDOL:Spinal column stenosis:0.57), (AMIODARONE HYDROCHLORIDE:Spinal column stenosis:0.5), (ATROPINE SULFATEDIPHENOXYLATE HYDROCHLORIDE:Spinal column stenosis:0.23), (BERAPROST SODIUM:Spinal column stenosis:0.08), (BISACODYL:Spinal column stenosis:0.01), (BISACODYL OR DOCUSATE SODIUM:Spinal column stenosis:0.69), (BRINZOLAMIDE:Spinal column stenosis:0.33), (CALCIUM CARBONATECHOLECALCIFEROL:Spinal column stenosis:1.12), (CANDESARTAN:Spinal column stenosis:1), (CANDESARTAN CILEXETILHYDROCHLOROTHIAZIDE:Spinal column stenosis:0.38), (CAPTOPRIL:Spinal column stenosis:0.18), (CARVEDILOL PHOSPHATE:Spinal column stenosis:0.71), (CIMETIDINE:Spinal column stenosis:0.1), (CIPROFLOXACIN:Spinal column stenosis:0.26), (CIPROFLOXACIN HYDROCHLORIDE:Spinal column stenosis:0.42), (CLOTRIMAZOLE:Spinal column stenosis:0.13), (DAPAGLIFLOZIN PROPANEDIOL:Spinal column stenosis:0.15), (DOFETILIDE:Spinal column stenosis:0.2), (DOXAZOSIN MESYLATE:Spinal column stenosis:0.38), (DRONABINOL:Spinal column stenosis:0.04), (ELETRIPTAN HYDROBROMIDE:Spinal column stenosis:0.75), (EMPAGLIFLOZINLINAGLIPTIN:Spinal column stenosis:0.22), (ENALAPRIL MALEATE:Spinal column stenosis:0.72), (EPINEPHRINE:Spinal column stenosis:0.25), (ERYTHROPOIETIN:Spinal column stenosis:0.04), (ESZOPICLONE:Spinal column stenosis:0.79), (ETORICOXIB:Spinal column stenosis:0.18), (EVEROLIMUS:Spinal column stenosis:0.81), (FINGOLIMOD HYDROCHLORIDE:Spinal column stenosis:0.6), (HYDROCHLOROTHIAZIDEIRBESARTAN:Spinal column stenosis:0.21), (HYDROXYCHLOROQUINE DIPHOSPHATE:Spinal column stenosis:0.24), (ILOPROST:Spinal column stenosis:0.32), (ISOSORBIDE:Spinal column stenosis:0.6), (LEDIPASVIRSOFOSBUVIR:Spinal column stenosis:0.46), (LISDEXAMFETAMINE DIMESYLATE:Spinal column stenosis:0.3), (LITHIUM CARBONATE:Spinal column stenosis:0.23), (METHENAMINE HIPPURATE:Spinal column stenosis:0.38), (METOLAZONE:Spinal column stenosis:0.2), (MOXIFLOXACIN HYDROCHLORIDE:Spinal column stenosis:0.07), (NEBIVOLOL:Spinal column stenosis:0.09), (PANTOPRAZOLE MAGNESIUM:Spinal column stenosis:0.31), (PAROXETINE:Spinal column stenosis:0.09), (PEGINTERFERON BETA-1A:Spinal column stenosis:0.24), (PROMETHAZINE HYDROCHLORIDE:Spinal column stenosis:0.3), (PROMETHAZINEPROMETHAZINE HYDROCHLORIDE:Spinal column stenosis:0.51), (QUETIAPINE:Spinal column stenosis:0.17), (RIZATRIPTAN BENZOATE:Spinal column stenosis:0.26), (ROPINIROLE:Spinal column stenosis:0.1), (SACUBITRILVALSARTAN:Spinal column stenosis:0.76), (SENNA LEAFSENNOSIDESSENNOSIDES A AND B:Spinal column stenosis:0.02), (SUMATRIPTAN:Spinal column stenosis:0.32), (SUMATRIPTANSUMATRIPTAN SUCCINATE:Spinal column stenosis:0.35), (TERIFLUNOMIDE:Spinal column stenosis:1.13), (TETRACYCLINE:Spinal column stenosis:0.06), (TOCILIZUMAB:Spinal column stenosis:0.2), (UMECLIDINIUM BROMIDEVILANTEROL TRIFENATATE:Spinal column stenosis:0.58), (VILAZODONE HYDROCHLORIDE:Spinal column stenosis:0.17), (ZINC:Spinal column stenosis:0.18), (AMIODARONE:Spinal compression fracture:0.09), (ANASTROZOLE:Spinal compression fracture:0.15), (BUPROPION HYDROCHLORIDE:Spinal compression fracture:0.22), (CERTOLIZUMAB PEGOL:Spinal compression fracture:0.54), (DEXLANSOPRAZOLE:Spinal compression fracture:0.24), (DILTIAZEM:Spinal compression fracture:0.06), (DILTIAZEM HYDROCHLORIDE:Spinal compression fracture:0.53), (DIPHENHYDRAMINE:Spinal compression fracture:0.04), (EVEROLIMUS:Spinal compression fracture:0.13), (IBRUTINIB:Spinal compression fracture:0.54), (INSULIN ASPART:Spinal compression fracture:0), (METRONIDAZOLE:Spinal compression fracture:0.54), (MYCOPHENOLATE MOFETILMYCOPHENOLATE MOFETIL HYDROCHLORIDE:Spinal compression fracture:0.15), (OMEPRAZOLE MAGNESIUM:Spinal compression fracture:0.26), (SECUKINUMAB:Spinal compression fracture:0.04), (THALIDOMIDE:Spinal compression fracture:0.13), (TOPIRAMATE:Spinal compression fracture:0.26), (ACETAMINOPHENCODEINE PHOSPHATE:Spinal cord injury:0.28), (ARMODAFINIL:Spinal cord injury:0.52), (BUDESONIDE:Spinal cord injury:0.65), (CLINDAMYCINCLINDAMYCIN PHOSPHATE:Spinal cord injury:0.06), (DIPHENHYDRAMINE:Spinal cord injury:0.56), (ELETRIPTAN HYDROBROMIDE:Spinal cord injury:0.04), (ENALAPRIL:Spinal cord injury:0.71), (EVOLOCUMAB:Spinal cord injury:0.46), (FERROUS SULFATEFERROUS SULFATE, DRIED:Spinal cord injury:0.32), (FINGOLIMOD HYDROCHLORIDE:Spinal cord injury:0.27), (INDOMETHACIN:Spinal cord injury:0.02), (INSULIN ASPART:Spinal cord injury:0.12), (LISDEXAMFETAMINE DIMESYLATE:Spinal cord injury:0.41), (METAXALONE:Spinal cord injury:0.42), (OLANZAPINE:Spinal cord injury:0.24), (PHENOBARBITAL:Spinal cord injury:0.1), (PRAMIPEXOLE:Spinal cord injury:0.08), (PRIMIDONE:Spinal cord injury:0.37), (RAMIPRIL:Spinal cord injury:0.04), (RANOLAZINE:Spinal cord injury:0.37), (TERIFLUNOMIDE:Spinal cord injury:0.82), (TOCILIZUMAB:Spinal cord injury:0.46), (VALSARTAN:Spinal cord injury:0.16), (VITAMIN B:Spinal cord injury:0.16), (ANASTROZOLE:Spinal disorder:0.02), (BUDESONIDE:Spinal disorder:0.23), (DIGOXIN:Spinal disorder:0.18), (ESZOPICLONE:Spinal disorder:0.12), (EVOLOCUMAB:Spinal disorder:0.56), (HYDROXYCHLOROQUINE DIPHOSPHATE:Spinal disorder:0.1), (LISDEXAMFETAMINE DIMESYLATE:Spinal disorder:0.11), (METHYLPREDNISOLONE SODIUM SUCCINATE:Spinal disorder:0.17), (PAROXETINE:Spinal disorder:0.32), (TERIFLUNOMIDE:Spinal disorder:0.07), (TOCILIZUMAB:Spinal disorder:0.36), (BUPROPION HYDROCHLORIDE:Spinal fracture:0.14), (DEXLANSOPRAZOLE:Spinal fracture:0.56), (EVOLOCUMAB:Spinal fracture:0.1), (IBRUTINIB:Spinal fracture:0.12), (NEBIVOLOL:Spinal fracture:0.21), (PANTOPRAZOLE:Spinal fracture:0.27), (PRAVASTATIN SODIUM:Spinal fracture:0.06), (VERAPAMIL HYDROCHLORIDE:Spinal fracture:0.31), (EVOLOCUMAB:Spinal operation:0.28), (SPIRONOLACTONE:Spinal operation:0.14), (ALENDRONIC ACID:Spinal osteoarthritis:0.19), (ANASTROZOLE:Spinal osteoarthritis:1.35), (BECLOMETHASONE DIPROPIONATE:Spinal osteoarthritis:0.23), (BISACODYL:Spinal osteoarthritis:0.09), (BISACODYL OR DOCUSATE SODIUM:Spinal osteoarthritis:0.54), (BORTEZOMIB:Spinal osteoarthritis:0.6), (BUDESONIDEFORMOTEROL FUMARATE:Spinal osteoarthritis:0.03), (BUPRENORPHINE HYDROCHLORIDENALOXONE HYDROCHLORIDE:Spinal osteoarthritis:0.24), (CARVEDILOL PHOSPHATE:Spinal osteoarthritis:0.08), (CEPHALEXIN:Spinal osteoarthritis:1.16), (CETIRIZINE HYDROCHLORIDE:Spinal osteoarthritis:1.06), (CHOLESTYRAMINE:Spinal osteoarthritis:0.9), (CIMETIDINE:Spinal osteoarthritis:0.1), (CLOTRIMAZOLE:Spinal osteoarthritis:0.26), (CLOZAPINE:Spinal osteoarthritis:0.43), (COLCHICINE:Spinal osteoarthritis:0.71), (COLESEVELAM HYDROCHLORIDE:Spinal osteoarthritis:0.11), (DACLIZUMAB:Spinal osteoarthritis:0.11), (DEVICE:Spinal osteoarthritis:0.24), (DIMENHYDRINATE:Spinal osteoarthritis:0.15), (DIPHENHYDRAMINE:Spinal osteoarthritis:0.91), (DOFETILIDE:Spinal osteoarthritis:0.04), (DOXEPIN HYDROCHLORIDE:Spinal osteoarthritis:0.03), (DUTASTERIDE:Spinal osteoarthritis:0.3), (EDOXABAN TOSYLATE:Spinal osteoarthritis:0.14), (ELETRIPTAN HYDROBROMIDE:Spinal osteoarthritis:0.59), (ENOXAPARIN:Spinal osteoarthritis:0.98), (EVEROLIMUS:Spinal osteoarthritis:0.7), (EVOLOCUMAB:Spinal osteoarthritis:1.11), (EZETIMIBESIMVASTATIN:Spinal osteoarthritis:0.31), (FINGOLIMOD HYDROCHLORIDE:Spinal osteoarthritis:0.44), (FLECAINIDE:Spinal osteoarthritis:0.22), (FLUOXETINEFLUOXETINE HYDROCHLORIDE:Spinal osteoarthritis:0.31), (FLUTICASONE:Spinal osteoarthritis:0.43), (HERBALSTURMERIC:Spinal osteoarthritis:0.2), (HYDROCHLOROTHIAZIDEIRBESARTAN:Spinal osteoarthritis:0.33), (HYDROXYCHLOROQUINE DIPHOSPHATE:Spinal osteoarthritis:0.53), (INDAPAMIDE:Spinal osteoarthritis:0.41), (INSULIN NOS:Spinal osteoarthritis:0.56), (IPRATROPIUM:Spinal osteoarthritis:0.33), (ISOSORBIDE:Spinal osteoarthritis:0.29), (LABETALOLLABETALOL HYDROCHLORIDE:Spinal osteoarthritis:0.09), (LERCANIDIPINE:Spinal osteoarthritis:0.39), (LERCANIDIPINE HYDROCHLORIDE:Spinal osteoarthritis:0.11), (LEVALBUTEROL HYDROCHLORIDE:Spinal osteoarthritis:0.13), (LITHIUM CARBONATE:Spinal osteoarthritis:0.79), (METHENAMINE HIPPURATE:Spinal osteoarthritis:0.17), (NADOLOL:Spinal osteoarthritis:0.17), (NILOTINIB:Spinal osteoarthritis:0.72), (OMEGA-3 FATTY ACIDS:Spinal osteoarthritis:0), (PANTOPRAZOLE MAGNESIUM:Spinal osteoarthritis:0.66), (PAROXETINE:Spinal osteoarthritis:0.45), (PAZOPANIB HYDROCHLORIDE:Spinal osteoarthritis:0.15), (PEGINTERFERON BETA-1A:Spinal osteoarthritis:0.4), (PENICILLIN:Spinal osteoarthritis:0.27), (PHENOBARBITAL:Spinal osteoarthritis:0.15), (PRAVASTATIN:Spinal osteoarthritis:1.26), (PRIMIDONE:Spinal osteoarthritis:0.68), (QUETIAPINE:Spinal osteoarthritis:0.46), (RANOLAZINE:Spinal osteoarthritis:0.64), (SACUBITRILVALSARTAN:Spinal osteoarthritis:0.84), (SCOPOLAMINE:Spinal osteoarthritis:0.07), (SULFAMETHOXAZOLE:Spinal osteoarthritis:0.19), (TERIFLUNOMIDE:Spinal osteoarthritis:0.63), (THALIDOMIDE:Spinal osteoarthritis:0.23), (TOLTERODINE TARTRATE:Spinal osteoarthritis:0.39), (UMECLIDINIUM BROMIDEVILANTEROL TRIFENATATE:Spinal osteoarthritis:0.16), (VANCOMYCIN:Spinal osteoarthritis:0.13), (ZOPICLONE:Spinal osteoarthritis:0.29), (ACARBOSE:Spinal pain:0.1), (ACENOCOUMAROL:Spinal pain:0.09), (ACETAZOLAMIDE:Spinal pain:0.02), (ADO-TRASTUZUMAB EMTANSINE:Spinal pain:0.02), (ALENDRONIC ACID:Spinal pain:0.16), (AMANTADINE:Spinal pain:0.19), (AZITHROMYCIN ANHYDROUS:Spinal pain:0.08), (BISACODYL OR DOCUSATE SODIUM:Spinal pain:0.71), (CARVEDILOL PHOSPHATE:Spinal pain:0.27), (CERTOLIZUMAB PEGOL:Spinal pain:1.61), (CHOLESTYRAMINE:Spinal pain:0.71), (CLOZAPINE:Spinal pain:0.39), (DALTEPARIN SODIUM:Spinal pain:0.15), (DAPAGLIFLOZIN PROPANEDIOL:Spinal pain:0.15), (DASATINIB:Spinal pain:1.08), (DIPHENHYDRAMINE:Spinal pain:0.72), (DOFETILIDE:Spinal pain:0.34), (DOXEPIN HYDROCHLORIDE:Spinal pain:0.03), (DRONABINOL:Spinal pain:0.15), (DUTASTERIDE:Spinal pain:0.86), (EDOXABAN TOSYLATE:Spinal pain:0.06), (EMPAGLIFLOZINLINAGLIPTIN:Spinal pain:0.13), (ESTROGENS, CONJUGATEDMEDROXYPROGESTERONE ACETATE:Spinal pain:0.17), (ESZOPICLONE:Spinal pain:0.78), (EVEROLIMUS:Spinal pain:0.83), (EVOLOCUMAB:Spinal pain:1.14), (EXEMESTANE:Spinal pain:0.19), (FERROUS SULFATEFERROUS SULFATE, DRIED:Spinal pain:1.81), (FLECAINIDE:Spinal pain:0.81), (FLUDROCORTISONE ACETATE:Spinal pain:0.04), (FLUTICASONE:Spinal pain:0.37), (FORMOTEROL FUMARATE:Spinal pain:0.25), (GLYCOPYRRONIUMINDACATEROL:Spinal pain:0.23), (HALOPERIDOL:Spinal pain:0.13), (HERBALSTURMERIC:Spinal pain:0.12), (HYDROCHLOROTHIAZIDEIRBESARTAN:Spinal pain:0.54), (HYDROXYCHLOROQUINE DIPHOSPHATE:Spinal pain:0.26), (HYDROXYUREA:Spinal pain:0.2), (HYOSCYAMINE:Spinal pain:0.2), (IBRUTINIB:Spinal pain:1.25), (ILOPROST:Spinal pain:0.04), (IMIPRAMINE HYDROCHLORIDE:Spinal pain:0.08), (INDAPAMIDEPERINDOPRIL:Spinal pain:0.92), (INFLUENZA VIRUS VACCINE:Spinal pain:0.38), (LABETALOLLABETALOL HYDROCHLORIDE:Spinal pain:0.65), (LEDIPASVIRSOFOSBUVIR:Spinal pain:0.35), (LEVALBUTEROL HYDROCHLORIDE:Spinal pain:0.18), (METFORMIN HYDROCHLORIDEVILDAGLIPTIN:Spinal pain:0.05), (METHENAMINE HIPPURATE:Spinal pain:0.26), (NALOXONE HYDROCHLORIDETILIDINE HYDROCHLORIDE:Spinal pain:0.07), (NEBIVOLOL HYDROCHLORIDE:Spinal pain:0.61), (NITROFURANTOIN:Spinal pain:0.91), (NITROFURANTOINNITROFURANTOIN MONOHYDRATE:Spinal pain:0.15), (OMEGA-3-ACID ETHYL ESTERS:Spinal pain:0.66), (PANTOPRAZOLE MAGNESIUM:Spinal pain:0.81), (PEGINTERFERON BETA-1A:Spinal pain:0.4), (PENICILLIN:Spinal pain:0.6), (PITAVASTATIN CALCIUM:Spinal pain:0.01), (RALOXIFENE HYDROCHLORIDE:Spinal pain:0.32), (RANOLAZINE:Spinal pain:0.9), (ROFLUMILAST:Spinal pain:0.24), (ROPINIROLE:Spinal pain:0.05), (SACUBITRILVALSARTAN:Spinal pain:1.14), (SCOPOLAMINE:Spinal pain:0.28), (TETRACYCLINE:Spinal pain:0.26), (THALIDOMIDE:Spinal pain:0.45), (TIOTROPIUM:Spinal pain:0.12), (TOLTERODINE TARTRATE:Spinal pain:0.58), (UMECLIDINIUM BROMIDEVILANTEROL TRIFENATATE:Spinal pain:0.15), (VILAZODONE HYDROCHLORIDE:Spinal pain:0.15), (ZINC:Spinal pain:0.15), (ACETAMINOPHENBUTALBITALCAFFEINE:Spondylitis:0.03), (ANASTROZO LE:Spondylitis:0.5), (ARMODAFINIL:Spondylitis:0.31), (BISACODYL OR DOCUSATE SODIUM:Spondylitis:0.11), (BUPROPION:Spondylitis:0.19), (CALCIUM CARBONATEVITAMIN D:Spondylitis:0.08), (CANDESARTAN CILEXETILHYDROCHLOROTHIAZIDE:Spondylitis:0.37), (CEPHALEXIN:Spondylitis:0.26), (CHOLESTYRAMINE:Spondylitis:0.6), (CIPROFLOXACIN HYDROCHLORIDE:Spondylitis:0.3), (DASATINIB:Spondylitis:0.01), (DIPHENHYDRAMINE: Spondylitis:0.69), (DOCUSATE SODIUM:Spondylitis:0.88), (ENOXAPARIN:Spondylitis:0.54), (ESZOPICLONE:Spondylitis:0.73), (EVEROLIMUS:Spondylitis:0.02), (EVOLOCUMAB:Spondylitis:0.46), (HEPARIN SODIUM:Spondylitis:0.2), (HYDROMORPHONE HYDROCHLORIDE:Spondylitis:0.15), (HYDROXYCHLOROQUINE DIPHOSPHATE:Spondylitis:0.51), (IBRUTINIB:Spondylitis:1.28), (INDAPAMIDE:Spondylitis: 0.09), (INDAPAMIDEPERINDOPRIL:Spondylitis:0.33), (LERCANIDIPINE:Spondylitis:0.26), (LERCANIDIPINE HYDROCHLORIDE:Spondylitis:0.2), (METAXALONE:Spondylitis:0.38), (METHYLPREDNIS OLONE SODIUM SUCCINATE:Spondylitis:0.38), (NILOTINIB:Spondylitis:0.01), (NITROFURANTOIN:Spondyli tis:0.1), (OLANZAPINE:Spondylitis:0.56), (OMEGA-3-ACID ETHYL ESTERS:Spondylitis:0.09), (OXCARBAZEPINE:Spondylitis:0.72), (OXYGEN:Spondylitis:0.05), (PANTOPRAZOLE MAGNESIUM:Spondylitis:0.3), (PENICILLIN:Spondylitis:0.29), (PRAMIPEXOLE DIHYDROCHLORIDE:Spondylitis:0.29), (PYRIDOXINE:Spondylitis:0.09), (QUETIAPINE:Sp ondylitis:0.19), (RABEPRAZOLE:Spondylitis:0.01), (RANOLAZINE:Spondylitis:0.41), (RIZAT RIPTAN BENZOATE:Spondylitis:0.04), (SACUBITRILVALSARTAN:Spondylitis:0.08), (VITAMIN B:Spondylitis:0.34), (CITALOPRAM HYDROBROMIDE:Spondyloarthropathy:0.47), (CORTISONEHYDROCORTISONE:Spondyl oarthropathy:0.05), (EVOLOCUMAB:Spondyloarthropathy:0.1), (LOVASTATIN:Spondyloarthr opathy:0.02), (POTASSIUM:Spondyloarthropathy:0.05), (TERIFLUNOMIDE:Spondyloarthropat hy:0.05), (UNSPECIFIED INGREDIENT:Spondyloarthropathy:0.26), (VARENICLINE TARTRATE:Spondyloarthropathy:0.07), (PYRAZINAMIDE:Sporotrichosis:0.55), (ADO-TRASTUZUMAB EMTANSINE:Squamous cell carcinoma:0.02), (ALBUTEROL SULFATE:Squamous cell carcinoma:0.41), (ANASTROZOLE:Squamous cell carcinoma:0.58), (BORTEZOMIB:Squamous cell carcinoma:0.18), (CANDESARTAN CILEXETILHYDROCHLOROTHIAZIDE:Squamous cell carcinoma:0.35), (CERTOLIZUMAB PEGOL:Squamous cell carcinoma:0.55), (CORTISONEHYDROCORTISONE:Squamous cell carcinoma:0.03), (CYCLOBENZAPRINE:Squamous cell carcinoma:0.02), (DASATINIB:Squamous cell carcinoma:0.4), (DESLORATADINE:Squamous cell carcinoma:0.02), (DEXLANSOPRAZOLE:Squamous cell carcinoma:0.58), (DILTIAZEM:Squamous cell carcinoma:0.26), (DILTIAZEM HYDROCHLORIDE:Squamous cell carcinoma:0.79), (ENALAPRIL:Squamous cell carcinoma:1.12), (EPIRUBICIN:Squamous cell carcinoma:0.36), (EVOLOCUMAB:Squamous cell carcinoma:0.16), (FLECAINIDE:Squamous cell carcinoma:0.09), (IDELALISIB:Squamous cell carcinoma:0.18), (IRINOTECAN HYDROCHLORIDE:Squamous cell carcinoma:0.49), (IRON:Squamous cell carcinoma:0.37), (LINAGLIPTIN:Squamous cell carcinoma:0.01), (METOPROLOL TARTRATE:Squamous cell carcinoma:0.42), (MOXIFLOXACIN HYDROCHLORIDE:Squamous cell carcinoma:0.17), (NIFEDIPINE:Squamous cell carcinoma:0.33), (NILOTINIB:Squamous cell carcinoma:0.57), (OXYGEN:Squamous cell carcinoma:0.17), (PANTOPRAZOLE MAGNESIUM:Squamous cell carcinoma:0.06), (PAZOPANIB HYDROCHLORIDE:Squamous cell carcinoma:0.36), (REGORAFENIB:Squamous cell carcinoma:0.59), (SACUBITRILVALSARTAN:Squamous cell carcinoma:0.15), (TAMSULOSIN:Squamous cell carcinoma:0.9), (THALIDOMIDE:Squamous cell carcinoma:0.25), (TOFACITINIB CITRATE:Squamous cell carcinoma:0.04), (TOPIRAMATE:Squamous cell carcinoma:0.33), (TORSEMIDE:Squamous cell carcinoma:0.48), (VINCRISTINE SULFATE:Squamous cell carcinoma:0.03), (ANASTROZOLE:Squamous cell carcinoma of head and neck:0.44), (BUMETANIDE:Squamous cell carcinoma of head and neck:0.36), (CALCIUMVITAMIN D:Squamous cell carcinoma of head and neck:0.06), (CANDESARTAN CILEXETILHYDROCHLOROTHIAZIDE:Squamous cell carcinoma of head and neck:0.2), (CEFTRIAXONE SODIUM:Squamous cell carcinoma of head and neck:0.09), (CERTOLIZUMAB PEGOL:Squamous cell carcinoma of head and neck:0.09), (CLONIDINE:Squamous cell carcinoma of head and neck:0.19), (DEXLANSOPRAZOLE:Squamous cell carcinoma of head and neck:0.72), (DILTIAZEM:Squamous cell carcinoma of head and neck:0.34), (DILTIAZEM HYDROCHLORIDE:Squamous cell carcinoma of head and neck:0.66), (EPINEPHRINE:Squamous cell carcinoma of head and neck:0.21), (ESZOPICLONE:Squamous cell carcinoma of head and neck:0.04), (IBRUTINIB:Squamous cell carcinoma of head and neck:0.63), (INDAPAMIDEPERINDOPRIL:Squamous cell carcinoma of head and neck:0.22), (IRINOTECAN:Squamous cell carcinoma of head and neck:0.44), (IRINOTECAN HYDROCHLORIDE:Squamous cell carcinoma of head and neck:0.21), (ISOSORBIDE MONONITRATE:Squamous cell carcinoma of head and neck:0.19), (LEVOLEUCOVORIN CALCIUM:Squamous cell carcinoma of head and neck:0.16), (METHYLPREDNISOLONE SODIUM SUCCINATE:Squamous cell carcinoma of head and neck:0.38), (NEBIVOLOL:Squamous cell carcinoma of head and neck:0.55), (NILOTINIB:Squamous cell carcinoma of head and neck:0.22), (PRAVASTATIN SODIUM:Squamous cell carcinoma of head and neck:0.14), (REGORAFENIB:Squamous cell carcinoma of head and neck:0.25), (TELMISARTAN:Squamous cell carcinoma of head and neck:0.2), (TIMOLOL:Squamous cell carcinoma of head and neck:0.25), (VERAPAMIL HYDROCHLORIDE:Squamous cell carcinoma of head and neck:0.39), (AMIODARONE HYDROCHLORIDE:Squamous cell carcinoma of lung:0.51), (ANASTROZOLE:Squamous cell carcinoma of lung:0.43), (CALCITRIOL:Squamous cell carcinoma of lung:0.01), (CALCIUM CARBONATECHOLECALCIFEROL:Squamous cell carcinoma of lung:0.67), (CANDESARTAN CILEXETILHYDROCHLOROTHIAZIDE:Squamous cell carcinoma of lung:0.13), (CLINDAMYCINCLINDAMYCIN PHOSPHATE:Squamous cell carcinoma of lung:0.04), (DEXLANSOPRAZOLE:Squamous cell carcinoma of lung:0.6), (DICLOFENAC:Squamous cell carcinoma of lung:0.2), (EPINEPHRINE:Squamous cell carcinoma of lung:0.31), (EVEROLIMUS:Squamous cell carcinoma of lung:0.26), (EVOLOCUMAB:Squamous cell carcinoma of lung:0.19), (FERROUS SULFATEFERROUS SULFATE, DRIED:Squamous cell carcinoma of lung:0.27), (FLECAINIDE:Squamous cell carcinoma of lung:0.03), (FLUTICASONE:Squamous cell carcinoma of lung:0.05), (IBRUTINIB:Squamous cell carcinoma of lung:0.58), (ILOPROST:Squamous cell carcinoma of lung:0.03), (NILOTINIB:Squamous cell carcinoma of lung:0.16), (PANTOPRAZOLE MAGNESIUM:Squamous cell carcinoma of lung:0.04), (PRAVASTATIN:Squamous cell carcinoma oflung:0.46), (RANOLAZINE:Squamous cell carcinoma of lung:0.06), (ZOLPIDEMZOLPIDEM TARTRATE:Squamous cell carcinoma of lung:0.27), (DILTIAZEM:Staphylococcal bacteraemia:0.16), (IBRUTINIB:Staphylococcal bacteraemia:0), (.ALPHA.1-PROTEINASE INHIBITOR HUMAN:Staphylococcal infection:0.4), (ACETAMINOPHENPROPOXYPHENE HYDROCHLORIDE:Staphylococcal infection:0.05), (ADO-TRASTUZUMAB EMTANSINE:Staphylococcal infection:0.04), (ALCOHOL:Staphylococcal infection:0.58), (ALUMINUM HYDROXIDEDIMETHICONEMAGNESIUM HYDROXIDE:Staphylococcal infection:0.13), (AMANTADINE:Staphylococcal infection:0.16), (AMLODIPINEVALSARTAN:Staphylococcal infection:0.09), (ANASTROZOLE:Staphylococcal infection:1.15), (APREPITANT:Staphylococcal infection:0.17), (ASPARAGINASE:Staphylococcal infection:0.44), (BELATACEPT:Staphylococcal infection:0.16), (BENZONATATE:Staphylococcal infection:0.05), (BEVACIZUMAB:Staphylococcal infection:0.03), (BUPRENORPHINE HYDROCHLORIDE:Staphylococcal infection:0.71), (BUPRENORPHINE HYDROCHLORIDENALOXONE HYDROCHLORIDE:Staphylococcal infection:0.08), (CALCIUM CARBONATEVITAMIN D:Staphylococcal infection:0.06), (CANDESARTAN:Staphylococcal infection:0.96), (CARVEDILOL PHOSPHATE:Staphylococcal infection:0.82), (CHLOROTHIAZIDE:Staphylococcal infection:0.1), (CHOLESTYRAMINE:Staphylococcal infection:1.29), (CIMETIDINE:Staphylococcal infection:0.02), (CLOMIPRAMINE HYDROCHLORIDE:Staphylococcal infection:0.27), (CLONIDINE HYDROCHLORIDE:Staphylococcal infection:0.01), (CODEINE:Staphylococcal infection:0.43), (CYPROHEPTADINE:Staphylococcal infection:0.66), (DABIGATRAN:Staphylococcal infection:0.49), (DACLIZUMAB:Staphylococcal infection:0.39), (DALTEPARIN:Staphylococcal infection:0.7), (DAPAGLIFLOZIN PROPANEDIOL:Staphylococcal infection:0.11), (DEXAMETHASONE ACETATE:Staphylococcal infection:0.13), (DIMENHYDRINATE:Staphylococcal infection:0.4), (DIMETHICONE:Staphylococcal infection:0.15), (DUTASTERIDE:Staphylococcal infection:0.75), (EDOXABAN TOSYLATE:Staphylococcal infection:0.89), (ELETRIPTAN HYDROBROMIDE:Staphylococcal infection:0.62), (ELOTUZUMAB:Staphylococcal infection:0.13), (ESTROGENS, CONJUGATEDMEDROXYPROGESTERONE ACETATE:Staphylococcal infection:0.02), (ESZOPICLONE:Staphylococcal infection:0.93), (ETORICOXIB:Staphylococcal infection:0.44), (EVEROLIMUS:Staphylococcal infection:0.49), (EVOLOCUMAB:Staphylococcal infection:0.89), (FENTANYL CITRATE:Staphylococcal infection:0.12), (FORMOTEROL FUMARATE:Staphylococcal infection:0.73), (FORMOTEROL FUMARATE\MOMETASONE FUROATE:Staphylococcal infection:0.2), (FULVESTRANT:Staphylococcal infection:0.03), (GLYCOPYRROLATE:Staphylococcal infection:0.16), (HUMAN PLATELET, ALLOGENIC:Staphylococcal infection:0.08), (HYDROCHLOROTHIAZIDETELMISARTAN:Staphylococcal infection:0.1), (HYDROXYCHLOROQUINE DIPHOSPHATE:Staphylococcal infection:0.11), (IBRITUMOMAB TIUXETAN:Staphylococcal infection:0.91), (IDELALISIB:Staphylococcal infection:0.32), (ILOPROST:Staphylococcal infection:0.74), (IMIPRAMINE HYDROCHLORIDE:Staphylococcal infection:0.11), (INDACATEROL:Staphylococcal infection:0.11), (INDAPAMIDE:Staphylococcal infection:0.5), (INOTUZUMAB OZOGAMICIN:Staphylococcal infection:0.21), (IVACAFTOR:Staphylococcal infection:0.21), (LABETALOL HYDROCHLORIDE:Staphylococcal infection:0.29), (LACTOBACILLUS RHAMNOSUS:Staphylococcal infection:0.19), (LEDIPASVIRSOFOSBUVIR:Staphylococcal infection:0.14), (LIDOCAINEPRILOCAINE:Staphylococcal infection:0.2), (LINAGLIPTIN:Staphylococcal infection:0.81), (METHENAMINE HIPPURATE:Staphylococcal infection:0.32), (METHENOLONE ENANTHATE:Staphylococcal infection:0.34), (METHYLPHENIDATE HYDROCHLORIDE:Staphylococcal infection:0.54), (METOLAZONE:Staphylococcal infection:0.26), (MOXONIDINE:Staphylococcal infection:0.07), (NABUMETONE:Staphylococcal infection:0.15), (NADOLOL:Staphylococcal infection:0.23), (NATEGLINIDE:Staphylococcal infection:0.25), (NILOTINIB:Staphylococcal infection:1.77), (OLODATEROL HYDROCHLORIDETIOTROPIUM BROMIDE MONOHYDRATE:Staphylococcal infection:0.13), (OXYBUTYNIN CHLORIDE:Staphylococcal infection:0.48), (PANCRELIPASE:Staphylococcal infection:0.31), (PANTOPRAZOLE MAGNESIUM:Staphylococcal infection:1.1), (PEMETREXED DISODIUM:Staphylococcal infection:0.17), (PENTAMIDINE:Staphylococcal infection:0.19), (PENTOSTATIN:Staphylococcal infection:0.24), (PHENYTOIN SODIUM:Staphylococcal infection:0.36), (PLANTAGO SEED:Staphylococcal infection:0.02), (PONATINIB:Staphylococcal infection:1.59), (PREDNISOLONE SODIUM PHOSPHATE:Staphylococcal infection:0.33), (PROGESTERONE:Staphylococcal infection:0.35), (PYRIDOXINE HYDROCHLORIDE:Staphylococcal infection:0.62), (RABEPRAZOLE:Staphylococcal infection:0.7), (RABEPRAZOLE SODIUM:Staphylococcal infection:0.62), (RALOXIFENE HYDROCHLORIDE:Staphylococcal infection:0.35), (RIVASTIGMINE:Staphylococcal infection:0.07), (RIVASTIGMINE TARTRATE:Staphylococcal infection:0.32), (ROFLUMILAST:Staphylococcal infection:0.62), (SALMETEROL:Staphylococcal infection:0.01), (SUCRALFATE:Staphylococcal infection:0.67), (TERBINAFINE HYDROCHLORIDE:Staphylococcal infection:0.43), (THALIDOMIDE:Staphylococcal infection:0.96), (TINZAPARIN:Staphylococcal infection:0.05), (TIOTROPIUM:Staphylococcal infection:0.74), (TOCOPHEROL:Staphylococcal infection:0.11), (TOLTERODINE TARTRATE:Staphylococcal infection:0.24), (TROSPIUM CHLORIDE:Staphylococcal infection:0.24), (UMECLIDINIUM BROMIDEVILANTEROL TRIFENATATE:Staphylococcal infection:0.49), (VARENICLINE TARTRATE:Staphylococcal infection:0.98), (VITAMIN A:Staphylococcal infection:0.26), (VITAMIN B:Staphylococcal infection:0.81), (ATRACURIUM:Status epilepticus:0.59), (CALCIUM:Status epilepticus:0.09), (CLINDAMYCINCLINDAMYCIN PHOSPHATE:Status epilepticus:0.33), (DALFAMPRIDINE:Status epilepticus:0.44), (DESFLURANE:Status epilepticus:0.33), (DIGOXIN:Status epilepticus:0.25), (DOCUSATE SODIUM:Status epilepticus:0.15), (ENALAPRIL:Status epilepticus:1.02), (ENALAPRIL MALEATE:Status epilepticus:0), (ESLICARBAZEPINE:Status epilepticus:0.18), (ESMOLOL HYDROCHLORIDE:Status epilepticus:0.59), (FERROUS SULFATEFERROUS SULFATE, DRIED:Status epilepticus:0.07), (HYDROMORPHONE HYDROCHLORIDE:Status epilepticus:0.11), (NEOSTIGMINE:Status epilepticus:1.12), (PRAVASTATIN SODIUM:Status epilepticus:0.09), (ROPIVACAINE:Status epilepticus:0.37), (SEVOFLURANE:Status epilepticus:0.64), (SUGAMMADEX:Status epilepticus:0.33), (ACETAMINOPHENCODEINE PHOSPHATE:Stem cell transplant:0.12), (ACLARUBICIN HYDROCHLORIDE:Stem cell transplant:0.25), (AMPHOTERICIN BDIMYRISTOYLPHOSPHATIDYLCHOLINE, DL-DIMYRISTOYLPHOSPHATIDYLGLYCEROL, DL-:Stem cell transplant:0.04), (ANIDULAFUNGIN:Stem cell transplant:0.26), (BELATACEPT:Stem cell transplant:0.24), (BRINCIDOFOVIR:Stem cell transplant:0.78), (BUMETANIDE:Stem cell transplant:0.26), (BUPROPION HYDROCHLORIDE:Stem cell transplant:0.08), (CALCITRIOL:Stem cell transplant:0.06), (CALCIUM CARBONATECHOLECALCIFEROL:Stem cell transplant:0.28), (CANAKINUMAB:Stem cell transplant:0.14), (CANDESARTAN:Stem cell transplant:0.09), (CANDESARTAN CILEXETIL:Stem cell transplant:0.08), (CEFOTAXIME SODIUM:Stem cell transplant:0.28), (CEPHALEXIN:Stem cell transplant:0.56), (CERTOLIZUMAB PEGOL:Stem cell transplant:0.55), (CHLORAMBUCIL:Stem cell transplant:0.49), (CHOLESTYRAMINE:Stem cell transplant:0.06), (COLCHICINE:Stem cell transplant:0.09), (CYCLOPHOSPHAMIDECYTARABINEDEXAMETHASONEDOXORUBI CINMETHOTREXATEVINCRISTINE:Stem cell transplant:0.13), (DACLIZUMAB:Stem cell transplant:0.32), (DEXLANSOPRAZOLE:Stem cell transplant:0.03), (DILTIAZEM:Stem cell transplant:0.13), (DILTIAZEM HYDROCHLORIDE:Stem cell transplant:0.05), (ENOXAPARIN:Stem cell transplant:0.41), (FERROUS SULFATE:Stem cell transplant:0.27), (FERROUS SULFATEFERROUS SULFATE, DRIED:Stem cell transplant:0.46), (GUSPERIMUS TRIHYDROCHLORIDE:Stem cell transplant:0.1), (IDELALISIB:Stem cell transplant:0.5), (LYMPHOCYTES:Stem cell transplant:0.28), (MELOXICAM:Stem cell transplant:0.08), (METHENOLONE ENANTHATE:Stem cell transplant:0.06), (OMEPRAZOLE MAGNESIUM:Stem cell transplant:0.13), (OSELTAMIVIR:Stem cell transplant:0.38), (PRAVASTATIN SODIUM:Stem cell transplant:0.29), (PREDNISOLONE SODIUM PHOSPHATE:Stem cell transplant:0.29), (PYRAZINAMIDE:Stem cell transplant:0.68), (SACUBITRILVALSARTAN:Stem cell transplant:0.09), (TELMISARTAN:Stem cell transplant:0.01), (THIOGUANINE:Stem cell transplant:0.24), (TRAZODONE HYDROCHLORIDE:Stem cell transplant:0.16), (VINDESINE SULFATE:Stem cell transplant:0.15), (WARFARIN SODIUM:Stem cell transplant:0.22), (ACETAMINOPHENOXYCODONEOXYCODONE HYDROCHLORIDE:Stent placement:0.69), (ACETAMINOPHENTRAMADOL:Stent placement:0.27), (AGALSIDASE BETA:Stent placement:0.33), (ALBIGLUTIDE:Stent placement:0.4), (ALENDRONIC ACID:Stent placement:0.61), (ALFACALCIDOL:Stent placement:0.49), (ALISKIREN:Stent placement:0.52), (AMLODIPINE BESYLATEATORVASTATIN CALCIUM:Stent placement:0.38), (AMLODIPINE BESYLATEHYDROCHLOROTHIAZIDEVALSARTAN:Stent placement:0.6), (AMLODIPINEVALSARTAN:Stent placement:0.53), (BETAMETHASONE DIPROPIONATECALCIPOTRIENECALCIPOTRIENE HYDRATE:Stent placement:0.01), (BETAMETHASONE DIPROPIONATECLOTRIMAZOLE:Stent placement:0.06), (BUDESONIDEFORMOTEROL FUMARATE:Stent placement:0.61), (BUPRENORPHINE HYDROCHLORIDE:Stent placement:0.19), (BUPRENORPHINE HYDROCHLORIDENALOXONE HYDROCHLORIDE:Stent placement:0.16), (BUTYLSCOPOLAMINE BROMIDE:Stent placement:0.19), (CALCITONIN SALMON:Stent placement:0.11), (CALCIUM\MAGNESIUM:Stent placement:0.15), (CALCIUM\MAGNESIUM\ZINC:Stent placement:0.55), (CANAGLIFLOZINMETFORMIN HYDROCHLORIDE:Stent placement:0.61), (CARBIDOPA:Stent placement:0.07), (CETIRIZINE HYDROCHLORIDEPSEUDOEPHEDRINE HYDROCHLORIDE:Stent placement:0.19), (CILASTATIN SODIUMIMIPENEM:Stent placement:0.17), (CINACALCET HYDROCHLORIDE:Stent placement:0.83), (CLIKSTAR:Stent placement:0.15), (CORTICOTROPIN:Stent placement:0.37), (DACLIZUMAB:Stent placement:0.15), (DALFAMPRIDINE:Stent placement:0.37), (DALTEPARIN:Stent placement:0.24), (DANAZOL:Stent placement:0.33), (DEFLAZACORT:Stent placement:0.56), (DEXTROAMPHETAMINE SULFATE:Stent placement:0.1), (DIGITOXIN:Stent placement:0.68), (DOMPERIDONE:Stent placement:0.33), (DONEPEZIL:Stent placement:0.38), (DOXEPIN HYDROCHLORIDE:Stent placement:0.66), (DRONABINOL:Stent placement:0.53), (DUTASTERIDETAMSULOSIN HYDROCHLORIDE:Stent placement:0.33), (ECONAZOLE:Stent placement:0.29), (ELETRIPTAN HYDROBROMIDE:Stent placement:1.16), (EMPAGLIFLOZINLINAGLIPTIN:Stent placement:1.06), (ESOMEPRAZOLE MAGNESIUMNAPROXEN:Stent placement:0.15), (ESTROGENS, CONJUGATEDMEDROXYPROGESTERONE ACETATE:Stent placement:0.65), (ETORICOXIB:Stent placement:0.63), (EVEROLIMUS:Stent placement:1.15), (FESOTERODINE FUMARATE:Stent placement:1.56), (FEXOFENADINEPSEUDOEPHEDRINE:Stent placement:0.22), (FLUDROCORTISONE:Stent placement:0.08), (FLUTICASONE FUROATE:Stent placement:0.29), (FLUTICASONE FUROATESALMETEROL XINAFOATE:Stent placement:0.6), (FORMOTEROL:Stent placement:0.04), (GLYCOPYRRONIUMINDACATEROL:Stent placement:0.64), (HALOPERIDOL:Stent placement:0.34), (HEPARIN CALCIUM:Stent placement:0.27), (HYDROCODONE BITARTRATEIBUPROFEN:Stent placement:0.21), (HYDROXYCHLOROQUINE DIPHOSPHATE:Stent placement:0.21), (HYOSCYAMINE SULFATE:Stent placement:0.08), (IDARUCIZUMAB:Stent placement:0.47), (ILOPROST:Stent placement:0.43), (INVESTIGATIONAL PRODUCT:Stent placement:0.37), (IODINE:Stent placement:0.16), (IXAZOMIB:Stent placement:0.17), (LACTASE:Stent placement:0.18), (LEFLUNOMIDE:Stent placement:0.01), (LIDOCAINEPRILOCAINE:Stent placement:0.34), (LITHIUM CARBONATE:Stent placement:0.7), (METFORMIN HYDROCHLORIDEROSIGLITAZONE MALEATE:Stent placement:0.82), (METHYLPREDNISOLONE ACETATE:Stent placement:1.19), (MINOCYCLINE HYDROCHLORIDE:Stent placement:0.09), (OLANZAPINE:Stent placement:1.11), (OLODATEROL HYDROCHLORIDETIOTROPIUM BROMIDE MONOHYDRATE:Stent placement:0.82), (ORPHENADRINE:Stent placement:0.18), (OXYBUTYNINOXYBUTYNIN CHLORIDE:Stent placement:0.08), (PANTOPRAZOLE MAGNESIUM:Stent placement:1.14), (PAZOPANIB HYDROCHLORIDE:Stent placement:0.42), (PEGINTERFERON BETA-1A:Stent placement:0.56), (PEMETREXED DISODIUM:Stent placement:0.5), (PHENOBARBITAL:Stent placement:0.95), (PILOCARPINE:Stent placement:0.2), (PIROXICAM:Stent placement:0.47), (PRAZOSIN:Stent placement:0.3), (PREDNISOLONEPREDNISOLONE ACETATEPREDNISOLONE SODIUM SUCCINATE:Stent placement:0.11), (PYRIDOXINE:Stent placement:0.13), (QUETIAPINE:Stent placement:1.11), (RABEPRAZOLE:Stent placement:0.88), (RALOXIFENE HYDROCHLORIDE:Stent placement:0.95), (RIZATRIPTAN BENZOATE:Stent placement:0.89), (SACCHAROMYCES CEREVISIAE:Stent placement:0.37), (SALMETEROL XINAFOATE:Stent placement:0.08), (SCOPOLAMINE:Stent placement:0.7), (SEVELAMER HYDROCHLORIDE:Stent placement:0.05), (SODIUM PHOSPHATE, DIBASIC\SODIUM PHOSPHATE, MONOBASIC:Stent placement:0.28), (SUMATRIPTAN:Stent placement:0.75), (TAPENTADOL HYDROCHLORIDE:Stent placement:0.04), (TERBINAFINE HYDROCHLORIDE:Stent placement:0.63), (TERBUTALINE SULFATE:Stent placement:0.58), (TETRACYCLINE:Stent placement:1.31), (THIOCTIC ACID:Stent placement:0.33), (THYROID, PORCINETHYROID, UNSPECIFIED:Stent placement:0.08), (TOCILIZUMAB:Stent placement:0.03), (TRANDOLAPRIL:Stent placement:0.13), (TRIMIPRAMINE MALEATE:Stent placement:0.05), (TROSPIUM CHLORIDE:Stent placement:0.73), (UMECLIDINIUM BROMIDE:Stent placement:0.02), (URSODIOL:Stent placement:0.32), (VALGANCICLOVIR HYDROCHLORIDE:Stent placement:0.1), (ZAFIRLUKAST:Stent placement:0.25), (ACARBOSE:Steroid therapy:0.03), (ACETAZOLAMIDE:Steroid therapy:0.16), (ALFACALCIDOL:Steroid therapy:0.28), (AMIODARONE HYDROCHLORIDE:Steroid therapy:0.71), (ARMODAFINIL:Steroid therapy:0.24), (BENAZEPRIL HYDROCHLORIDE:Steroid therapy:0.42), (BENDAMUSTINE HYDROCHLORIDE:Steroid therapy:0.17), (BENZONATATE:Steroid therapy:0.05), (BIOTIN:Steroid therapy:0.02), (BISACODYL OR DOCUSATE SODIUM:Steroid therapy:0.73), (BROMAZEPAM:Steroid therapy:0.05), (BUDESONIDEFORMOTEROL FUMARATE:Steroid therapy:0.42), (BUSPIRONE HYDROCHLORIDE:Steroid therapy:0.51), (CALCIUM CITRATE:Steroid therapy:0.35), (CANDESARTAN CILEXETILHYDROCHLOROTHIAZIDE:Steroid therapy:0.58), (CARVEDILOL PHOSPHATE:Steroid therapy:0.48), (CASPOFUNGIN:Steroid therapy:0.32), (CLINDAMYCIN:Steroid therapy:0.78), (CLOZAPINE:Steroid therapy:0.6), (COLCHICINE:Steroid therapy:0.87), (DACLIZUMAB:Steroid therapy:0.51), (DASATINIB:Steroid therapy:0.76), (DEVICE:Steroid therapy:0.19), (DICYCLOMINE HYDROCHLORIDE:Steroid therapy:0.46), (DIPYRONE:Steroid therapy:0.18), (DUTASTERIDE:Steroid therapy:0.27), (ECONAZOLE:Steroid therapy:0.14), (ESZOPICLONE:Steroid therapy:0.76), (ETORICOXIB:Steroid therapy:0.31), (EVOLOCUMAB:Steroid therapy:0.75), (EZETIMIBESIMVASTATIN:Steroid therapy:0.25), (FESOTERODINE FUMARATE:Steroid therapy:0.1), (FLECAINIDE:Steroid therapy:0.61), (FLUTICASONE:Steroid therapy:0.6), (FORMOTEROL FUMARATE\MOMETASONE FUROATE:Steroid therapy:0.15), (GLYCOPYRROLATE:Steroid therapy:0.03), (HYDROCHLOROTHIAZIDEIRBESARTAN:Steroid therapy:0.21), (HYDROCHLOROTHIAZIDEVALSARTAN:Steroid therapy:0.67), (HYDROXYCHLOROQUINE DIPHOSPHATE:Steroid therapy:0.21), (IDELALISIB:Steroid therapy:0.21), (ILOPROST:Steroid therapy:0.46), (IMATINIB MESYLATE:Steroid therapy:0.74), (IMIPRAMINE HYDROCHLORIDE:Steroid therapy:0.19), (INDAPAMIDE:Steroid therapy:0.3), (INDAPAMIDEPERINDOPRIL:Steroid therapy:0.18), (IPRATROPIUM:Steroid therapy:0.77), (IPRATROPIUM BROMIDE:Steroid therapy:0.16), (ISOSORBIDE:Steroid therapy:0.43), (ITRACONAZOLE:Steroid therapy:0.22), (LERCANIDIPINE:Steroid therapy:0.49), (LERCANIDIPINE HYDROCHLORIDE:Steroid therapy:0.41), (LIDOCAINEPRILOCAINE:Steroid therapy:0.34), (NABUMETONE:Steroid therapy:0.05), (NALOXONE HYDROCHLORIDEOXYCODONE HYDROCHLORIDE:Steroid therapy:0.46), (NEBIVOLOL:Steroid therapy:0.74), (NITROFURANTOIN:Steroid therapy:0.8), (OLANZAPINE:Steroid therapy:0.95), (OMEGA-3-ACID ETHYL ESTERS:Steroid therapy:0.38), (OXYBUTYNIN CHLORIDE:Steroid therapy:0.04), (PAZOPANIB HYDROCHLORIDE:Steroid therapy:0.03), (PEGINTERFERON BETA-1A:Steroid therapy:0.12), (PONATINIB:Steroid therapy:0.1), (PYRIDOXINE HYDROCHLORIDE:Steroid therapy:0.22), (RABEPRAZOLE:Steroid therapy:0.33), (RIZATRIPTAN BENZOATE:Steroid therapy:0.16), (ROFLUMILAST:Steroid therapy:0.45), (SACUBITRILVALSARTAN:Steroid therapy:0.76), (SODIUM OXYBATE:Steroid therapy:0.49), (SOTALOL:Steroid therapy:0.68), (SULFAMETHOXAZOLE:Steroid therapy:0.5), (SUMATRIPTANSUMATRIPTAN SUCCINATE:Steroid therapy:0.16), (TETRACYCLINE:Steroid therapy:0.02), (TOCILIZUMAB:Steroid therapy:0.88), (TOLTERODINE TARTRATE:Steroid therapy:0.24), (UMECLIDINIUM BROMIDEVILANTEROL TRIFENATATE:Steroid therapy:0.5), (VANCOMYCIN HYDROCHLORIDE:Steroid therapy:0.75), (ZONISAMIDE:Steroid therapy:0.2), (.ALPHA.1-PROTEINASE INHIBITOR HUMAN:Stomatitis:0.15), (ABIRATERONE ACETATE:Stomatitis:0.19), (ACARBOSE:Stomatitis:0.52), (ACENOCOUMAROL:Stomatitis:0), (ACETAMINOPHENOXYCODONEOXYCODONE HYDROCHLORIDE:Stomatitis:0.37), (ACTIVATED CHARCOAL:Stomatitis:0.06), (ALCOHOL:Stomatitis:0.07), (ALDESLEUKIN:Stomatitis:0.03), (ALEMTUZUMAB:Stomatitis:0.3), (ALENDRONIC ACID:Stomatitis:0.73), (AMILORIDE HYDROCHLORIDEHYDROCHLOROTHIAZIDE:Stomatitis:0.03), (AMINO ACIDSDEXTROSEELECTROLYTES NOS:Stomatitis:0.03), (AMIODARONE HYDROCHLORIDE:Stomatitis:0.99), (AMOXAPINE:Stomatitis:0.21), (AMPICILLIN:Stomatiti s:0.58), (ANASTROZOLE:Stomatitis:2.56), (ASPARAGINASE:Stomatitis:0.52), (AZELASTINE: Stomatitis:0.07), (BENAZEPRILBENAZEPRIL HYDROCHLORIDE:Stomatitis:0.5), (BEPRIDIL:Stomatitis:0.1), (BICALUTAMIDE:Stomatitis: 0.52), (BRIMONIDINE TARTRATE:Stomatitis:0.01), (BUDESONIDEFORMOTEROL FUMARATE:Stomatitis:0.25), (BUPRENORPHINE HYDROCHLORIDE:Stomatitis:0.14), (BUPRENORPHINE HYDROCHLORIDENALOXONE HYDROCHLORIDE:Stomatitis:0.29), (CALCIPOTRIENE:Stomatitis:0.03), (CALCITONIN:Sto matitis:0.01), (CALCITONIN SALMON:Stomatitis:0.84), (CALCIUM CARBONATEVITAMIN D:Stomatitis:0.15), (CALCIUM CITRATE:Stomatitis:0.73), (CANAGLIFLOZINMETFORMIN HYDROCHLORIDE:Stomatitis:0.21), (CARVEDILOL PHOSPHATE:Stomatitis:0.43), (CEFEPIMECEFEPIME HYDROCHLORIDE:Stomatitis:0.26), (CEPHARANTHINE:Stomatitis:0.21), (CHLORAMBUCI L:Stomatitis:0.72), (CHLORTHALIDONE:Stomatitis:0.31), (CICLESONIDE:Stomatitis:0.34), (C LOZAPINE:Stomatitis:1.22), (DABRAFENIB:Stomatitis:1.14), (DABRAFENIB MESYLATE:Stomatitis:0.19), (DASATINIB:Stomatitis:2.08), (DESVENLAFAXINE SUCCINATE:Stomatitis:0.22), (DEVICE:Stomatitis:0.2), (DEXMEDETOMIDINE HYDROCHLORIDE:Stomatitis:0.1), (DIGITOXIN:Stomatitis:0.08), (DILAZEP:Stomatitis:0.14), (DIPYRONE:Stomatitis:0.46), (DOBUTAMINE HYDROCHLORIDE:Stomatitis:0.2), (DOCUSATE CALCIUM:Stomatitis:0.49), (DOFETILIDE:Stomatitis:0.21), (DOLASETRON MESYLATE:Stomatitis:0.15), (DOPAMINE HYDROCHLORIDE:Stomatitis:0.41), (DOXAZOSINDOXAZOSIN MESYLATE:Stomatitis:0.31), (DUTASTERIDE:Stomatitis:0.83), (ECONAZOLE:Stomatitis:0.19), (EDOXABAN TOSYLATE:Stomatitis:1), (ELETRIPTAN HYDROBROMIDE:Stomatitis:0.88), (ELOTUZUMAB:Stomatitis:0.63), (EPLERENONE:Stoma titis:0.38), (ERIBULIN:Stomatitis:0.64), (ERIBULIN MESYLATE:Stomatitis:0.12), (ESTROGENS, CONJUGATEDMEDROXYPROGESTERONE ACETATE:Stomatitis:0.15), (ETORICOXIB:Stomatitis:0.71), (ETRETINATE:Stomatitis:1.25), (EVOLOCUMAB:Stomatitis:0.76), (FESOTERODINE FUMARATE:Stomatitis:0.48), (FLUDROCORTISONE ACETATE:Stomatitis:0.31), (FLUOCINONIDE:Stomatitis:0.52), (FLUOXETINEFLUOXETIN E HYDROCHLORIDE:Stomatitis:0.55), (FLUTICASONE FUROATESALMETEROL XINAFOATE:Stomatitis:0.86), (FLUVASTATIN SODIUM:Stomatitis:0.08), (FORMOTEROL FUMARATE:Stomatitis:0.67), (FORMOTEROL FUMARATE\MOMETASONE FUROATE:Stomatitis:0.03), (GLYCOPYRRONIUMINDACATEROL:Stomatitis:0.54), (HERB ALSTURMERIC:Stomatitis:0.29), (HYDROCHLOROTHIAZIDETELMISARTAN:Stomatitis: 0.05), (HYDROXYZINE PAMOATE:Stomatitis:1.04), (IBRITUMOMAB TIUXETAN:Stomatitis:0.7), (IDELALISIB:Stomatitis:0.76), (ILOPROST:Stomatitis:0.53), (IMID APRIL:Stomatitis:0.05), (IMIPRAMINE HYDROCHLORIDE:Stomatitis:0.49), (INDACATEROL:Stomatitis:0.11), (INDAPAMIDE:Sto matitis:0.8), (INDAPAMIDEPERINDOPRIL:Stomatitis:0.46), (INFLIXIMAB-DYYB:Stomatitis:0.13), (INTERFERON ALFA-2A:Stomatitis:0.02), (INTERFERON ALFA-2B:Stomatitis:0.46), (ISOSORBIDE:Stomatitis:0.71), (IXAZOMIB:Stomatitis:0.44), (LACOSAM IDE:Stomatitis:0.05), (LENVATINIB:Stomatitis:0.32), (LEVALBUTEROL:Stomatitis:0.15), (LI OTHYRONINE SODIUM:Stomatitis:0.06), (LISDEXAMFETAMINE DIMESYLATE:Stomatitis:0.12), (LITHIUM CARBONATE:Stomatitis:0.99), (MECLIZINEMECLIZINE HYDROCHLORIDE:Stomatitis:0.28), (MEDROXYPROGESTERONE ACETATE:Stomatitis:0.26), (MEPERIDINE HYDROCHLORIDE:Stomatitis:0.34), (METAXALONE:Stomatitis:0.6), (METFORMIN HYDROCHLORIDEVILDAGLIPTIN:Stomatitis:0.29), (METHENAMINE HIPPURATE:Stomatitis:0.44), (METHIMAZOLE:Stomatitis:0.32), (MEXILETINE HYDROCHLORIDE:Stomatitis:0.03), (MITIGLINIDE:Stomatitis:0.03), (MIZORIBINE:Stomatit is:0.41), (MODAFINIL:Stomatitis:0.23), (NADOLOL:Stomatitis:0.15), (NALOXONE HYDROCHLORIDETILIDINE HYDROCHLORIDE:Stomatitis:0.01), (NATEGLINIDE:Stomatitis:0.04), (NOREPINEPHRINE: Stomatitis:0.16), (NORTRIPTYLINE:Stomatitis:0.42), (OLODATEROL HYDROCHLORIDETIOTROPIUM BROMIDE MONOHYDRATE:Stomatitis:0.54), (OSELTAMIVIR:Stomatitis:0.26), (PANTOPRAZOLE MAGNESIUM:Stomatitis:1.37), (PEGINTERFERON BETA-1A:Stomatitis:0.16), (PENICILLIN:Stomatitis:0.75), (PENICILLIN V:Stomatitis:0.37), (PENTOSTATIN:Stomatitis:0.49), (PHENYTOIN:Stomatitis:0.13), (POLYET HYLENE GLYCOL 4000:Stomatitis:0.04), (POTASSIUM CITRATETRISODIUM CITRATE DIHYDRATE:Stomatitis:0.37), (PREDNISOLONE SODIUM PHOSPHATE:Stomatitis:0.4), (PROPRANOLOLPROPRANOLOL HYDROCHLORIDE:Stomatitis:0.07), (RADIATION THERAPY:Stomatitis:0.84), (RANOLAZINE:Stomatitis:0.75), (RIZATRIPTAN BENZOATE:Stomatitis:0.75), (ROPINIROLE HYDROCHLORIDE:Stomatitis:0.36), (SARPOGRELATE:Stomatitis:0.67), (SILODOSIN:Stom atitis:0.11), (SOTALOL:Stomatitis:0.97), (TETRACYCLINE:Stomatitis:0.31), (TIGECYCLINE: Stomatitis:0.38), (TINZAPARIN:Stomatitis:0.39), (TIOTROPIUM:Stomatitis:0.57), (TIPEPIDINE HIBENZATE:Stomatitis:0.13), (TOCOPHEROL:Stomatitis:0.04), (TRAMETINIB:Stomatitis:1.21), (UMECLIDINIUM BROMIDE:Stomatitis:0.05), (VARENICLINE TARTRATE:Stomatitis:0.73), (VINDESINE SULFATE:Stomatitis:0.02), (VINORELBINEVINORELBINE TARTRATE:Stomatitis:0.22), (VITAMIN B:Stomatitis:1.06), (.ALPHA.1-PROTEINASE INHIBITOR HUMAN:Stress:0.17), (ACETAMINOPHENOXYCODONEOXYCODONE HYDROCHLORIDE:Stress:0.53), (AMANTADINE HYDROCHLORIDE:Stress:0.39), (AMISULPRIDE:Stress:0.43), (AMLODIPINEVALSARTA N:Stress:0.23), (AZELASTINE:Stress:0.13), (AZELASTINE HYDROCHLORIDE:Stress:0.16), (BETAMETHASONE DIPROPIONATECALCIPOTRIENECALCIPOTRIENE HYDRATE:Stress:0.03), (BRINZOLAMIDE:Stress:0.3), (BUTYLSCOPOLAMINE BROMIDE:Stress:0.21), (CALCIPOTRIENE:Stress:0.01), (CALCITONIN SALMON:Stress:0.03), (CALCIUM CARBONATEVITAMIN D:Stress:0.53), (CANAKINUMAB:Stress:0.07), (CARVEDILOL PHOSPHATE:Stress:1.11), (CHONDROITIN SULFATE (BOVINE)GLUCOSAMINE HYDROCHLORIDE:Stress:0.34), (CICLESONIDE:Stress:0.62), (CINACALCET HYDROCHLORIDE:Stress:0.01), (CLINDAMYCIN:Stress:0.56), (CORTICOTROPIN:Stress:0.46), (DABIGATRAN:Stress:0.06), (DACLIZUMAB:Stress:0.75), (DASATINIB:Stress:0.87), (DI PYRONE:Stress:0.25), (DRONABINOL:Stress:0.7), (DRONEDARONE:Stress:0.04), (DUTAST ERIDE:Stress:0.89), (EDOXABAN TOSYLATE:Stress:0.09), (EMPAGLIFLOZINLINAGLIPTIN:Stress:0.73), (EPLERENONE:Str ess:0.28), (ERYTHROMYCIN:Stress:0.23), (ERYTHROPOIETIN:Stress:0.54), (ESOMEPRAZOLE MAGNESIUMNAPROXEN:Stress:0.33), (FEBUXOSTAT:Stress:0.47), (FEXOFENADINEPS EUDOEPHEDRINE:Stress:0.14), (FLUTICASONE:Stress:0.75), (FORMOTEROL FUMARATE:Stress:0.57), (FORMOTEROL FUMARATE\MOMETASONE FUROATE:Stress:0.28), (GLYCOPYRRONIUMINDACATEROL:Stress:0.36), (GUANFACIN E:Stress:0.5), (GUANFACINE HYDROCHLORIDE:Stress:0.1), (HYDROCHLOROTHIAZIDELOSARTAN:Stress:0.05), (ILO PROST:Stress:0.13), (INSULIN DEGLUDEC:Stress:0.43), (LENALIDOMIDE:Stress:0.45), (LERCANIDIPINE HYDROCHLORIDE:Stress:0.56), (LIDOCAINE HYDROCHLORIDE:Stress:0.48), (LIOTHYRONINE:Stress:0.02), (MEGESTROL ACETATE:Stress:0.2), (MEPOLIZUMAB:Stress:0.1), (METAXALONE:Stress:1.51), (METFOR MIN HYDROCHLORIDEVILDAGLIPTIN:Stress:0.09), (METHYLPREDNISOLONE SODIUM SUCCINATE:Stress:1.28), (MIDODRINE:Stress:0.4), (MUPIROCINMUPIROCIN CALCIUM:Stress:0.16), (NALOXONE HYDROCHLORIDEOXYCODONE HYDROCHLORIDE:Stress:0.94), (NATEGLINIDE:Stress:0.12), (NILOTINIB:Stress:0.79), (NI NTEDANIB:Stress:0.08), (OSPEMIFENE:Stress:0), (PEGINTERFERON BETA-1A:Stress:0.93), (PENTOSAN POLYSULFATE SODIUM:Stress:0.19), (PERINDOPRIL:Stress:0.62), (PONATINIB:Stress:0.16), (PREDNISOLO NE ACETATE:Stress:0.28), (PSYLLIUM HUSK:Stress:0.3), (SACUBITRILVALSARTAN:Stress:0.56), (SALMETEROL XINAFOATE:Stress:0.05), (SULFAMETHOXAZOLE:Stress:0.14), (TERBINAFINE HYDROCHLORIDE:Stress:0.27), (TETRACYCLINE:Stress:0.69), (TIOTROPIUM:Stress:0.3), (TOCOPHEROL:Stress:0.08), (UMECLIDINIUM BROMIDE:Stress:0.06), (UMECLIDINIUM BROMIDEVILANTEROL TRIFENATATE:Stress:1.01), (ZAFIRLUKAST:Stress:0.05), (ZIPRASIDONE HYDROCHLORIDEZIPRASIDONE MESYLATE:Stress:0.41), (BUPRENORPHINE HYDROCHLORIDE:Suicidal ideation:0.44), (CERTOLIZUMAB PEGOL:Suicidal ideation:0.28), (CHLORPROMAZINE:Suicidal ideation:0.03), (CYCLOBENZAPRINE HYDROCHLORIDE:Suicidal ideation:0.11), (ENALAPRIL:Suicidal ideation:0.08), (ESOMEPRAZOLE MAGNESIUM:Suicidal ideation:0.64), (FOLIC ACID:Suicidal ideation:1.46), (INDOMETHACIN:Suicidal ideation:0.01), (PANTOPRAZOLE:Suicidal ideation:0), (PROGESTERONE:Suicidal ideation:0.02), (TERIFLUNOMIDE:Suicidal ideation:0.35), (WARFARIN:Suicidal ideation:0.44), (BUPRENORPHINE HYDROCHLORIDENALOXONE HYDROCHLORIDE:Suicide attempt:0.78), (CALCIUM CARBONATE:Suicide attempt:0.17), (CALCIUM CARBONATECHOLECALCIFEROL:Suicide attempt:0.95), (CERTOLIZUMAB PEGOL:Suicide attempt:0.87), (CHOLESTYRAMINE:Suicide attempt:0.36), (CLINDAMYCINCLINDAMYCIN PHOSPHATE:Suicide attempt:0.09), (ELETRIPTAN HYDROBROMIDE:Suicide attempt:0.07), (ENOXAPARIN:Suicide attempt:0.7), (EVOLOCUMAB:Suicide attempt:0.68), (FEBUXOSTAT:Suicide attempt:0.05), (FERROUS SULFATEFERROUS SULFATE, DRIED:Suicide attempt:1.15), (HYDROCHLOROTHIAZIDETELMISARTAN:Suicide attempt:0.06), (HYOSCYAMINE:Suicide attempt:0.14), (IBRUTINIB:Suicide attempt:0.67), (INDAPAMIDEPERINDOPRIL:Suicide attempt:0.26), (LEDIPASVIRSOFOSBUVIR:Suicide attempt:0.16), (LERCANIDIPINE HYDROCHLORIDE:Suicide attempt:0.19), (LOVASTATIN:Suicide attempt:1.68), (METRONIDAZOLEMETRONIDAZOLE HYDROCHLORIDE:Suicide attempt:0.15), (NITROFURANTOIN:Suicide attempt:0.36), (OMEPRAZOLE MAGNESIUM:Suicide attempt:0.49), (OXYGEN:Suicide attempt:0.11), (PALIPERIDONE PALMITATE:Suicide attempt:0.14), (POLYETHYLENE GLYCOLS:Suicide attempt:0.37), (PRAVASTATIN:Suicide attempt:0.73), (PRAVASTATIN SODIUM:Suicide attempt:0.88), (PROBIOTICS NOS:Suicide attempt:0.11), (RIZATRIPTAN BENZOATE:Suicide attempt:0.16), (ROPINIROLE HYDROCHLORIDE:Suicide attempt:0.3), (SCOPOLAMINE:Suicide attempt:0.17), (TIOTROPIUM BROMIDE MONOHYDRATE:Suicide attempt:0.31), (TIZANIDINE HYDROCHLORIDE:Suicide attempt:0.58), (TOFACITINIB CITRATE:Suicide attempt:0.25), (VILAZODONE HYDROCHLORIDE:Suicide attempt:0.07), (ZIPRASIDONE HYDROCHLORIDEZIPRASIDONE MESYLATE:Suicide attempt:0.01), (.ALPHA.-TOCOPHEROL ACETATE, D-ASCORBIC ACIDCALCIUM PANTOTHENATEFOLIC ACIDMANGANESENIACINAMIDEPYRIDOXINE\RIBOFLAVINTHIAMINEVITAMIN A\ZINC OXIDE:Supplementation therapy:0.01), (.ALPHA.-TOCOPHEROLVITAMINS:Supplementation therapy:0.42), (.ALPHA.1-PROTEINASE INHIBITOR HUMAN:Supplementation therapy:2.12), (.BETA.-HYDROXYISOVALERIC ACIDARGININEGLUTAMINE:Supplementation therapy:0.63), (ABOBOTULINUMTOXINA:Supplementation therapy:1.1), (ACECLOFENAC:Supplementation therapy:0.12), (ACETAMINOPHEN\CAFFEINECARISOPRODOLDICLOFENAC:Supplemen tation therapy:0.04), (ACETAMINOPHEN\CAFFEINECARISOPRODOLDICLOFENAC SODIUM:Supplementation therapy:0.09), (ACETAMINOPHEN\CAFFEINEOPIUM:Supplementation therapy:0.02), (ACETAMINOPHENCHLORPHENIRAMINE MALEATEPSEUDOEPHEDRINE HYDROCHLORIDE:Supplementation therapy:0.35), (ACETAMINOPHENCHLORZOXAZONE:Supplementation therapy:0.66), (ACRIVASTINEPSEUDOEPHEDRINE HYDROCHLORIDE:Supplementation therapy:0.1), (ACTIVATED CHARCOAL:Supplementation therapy:0.94), (ALBUTEROLIPRATROPIUM BROMIDE:Supplementation therapy:0.42), (ALOSETRON HYDROCHLORIDE:Supplementation therapy:0.25), (ALUMINUM CHLORIDE:Supplementation therapy:0.32), (AMINOBENZOIC ACIDCALCIUM PANTOTHENATECYSTINEKERATINTHIAMINE MONONITRATE:Supplementation therapy:0.04), (AMISULPRIDE:Supplementation therapy:1.62), (AMITRIPTYLINEPERPHENAZINE:Supplementation therapy:0.2), (AMLODIPINE BESYLATEATORVASTATIN CALCIUM TRIHYDRATE:Supplementation therapy:0.08), (AMLODIPINEVALSARTAN:Supplementation therapy:1.84), (AMMONIUM CHLORIDEDEXTROMETHORPHAN\SODIUM CITRATE:Supplementation therapy:1.25), (AMPHETAMINE ASPARTATEDEXTROAMPHETAMINE SACCHARATE:Supplementation therapy:0.02), (AMPHETAMINE SULFATE:Supplementation therapy:0.27), (ANHYDROUS CITRIC ACIDMAGNESIUM OXIDE\SODIUM PICOSULFATE:Supplementation therapy:0.04), (ANIDULAFUNGIN:Supplementation therapy:0.42), (ARCTOSTAPHYLOS UVA-URSI LEAF\URTICA DIOICA:Supplementation therapy:0.02), (ARIPIPRAZOLE LAUROXIL:Supplementation therapy:0.92), (ASCORBIC ACID/CHROMIUM/SELENIUM/VANADYL SULFATE:Supplementation therapy:0.73), (ASPARAGINASE:Supplementation therapy:0.75), (ASPIRINBUTALBITALCAFFEINECODEINE PHOSPHATE:Supplementation therapy:0.36), (ASPIRIN\CAFFEINESALICYLAMIDE:Supplementation therapy:0.94), (ASPIRINCLOPIDOGREL:Supplementation therapy:0.05), (ASPIRINMAGNESIUM OXIDE:Supplementation therapy:0.6), (ATAZANAVIRRITONAVIR:Supplementation therapy:0.16), (ATORVASTATIN CALCIUMEZETIMIBE:Supplementation therapy:0.24), (ATRACURIUM:Supplementation therapy:0.41), (ATRACURIUM BESYLATE:Supplementation therapy:0.04), (ATROPINE SULFATEDIPHENOXYLATE:Supplementation therapy:0.14), (AZITHROMYCIN MONOHYDRATE:Supplementation therapy:0.72), (BAMIFYLLINE HYDROCHLORIDE:Supplementation therapy:0.57), (BECLOMETHASONEFORMOTEROL:Supplementation therapy:0.8), (BELATACEPT:Supplementation therapy:1.31), (BENSERAZIDELEVODOPA:Supplementation therapy:0.15), (BEPOTASTINEBEPOTASTINE BESILATE:Supplementation therapy:0.14), (BETAMETHASONE DIPROPIONATECALCIPOTRIENE HYDRATE:Supplementation therapy:0.34), (BISACODYL OR GLYCERIN OR MINERAL OIL OR SODIUM PHOSPHATE, DIBASIC\SODIUM PHOSPHATE, MONOBASIC:Supplementation therapy:0), (BISACODYLGLYCERINMINERAL OILSODIUM PHOSPHATE, DIBASIC\SODIUM PHOSPHATE, MONOBASIC:Supplementation therapy:0.76), (BLESELUMAB:Supplementation therapy:0.58), (BOCEPREVIR:Supplementation therapy:1.04), (BORIC ACID:Supplementation therapy:0.24), (BORON:Supplementation therapy:0.34), (BOSUTINIB MONOHYDRATE:Supplementation therapy:0.21), (BROMOPRIDE:Supplementation therapy:0.15), (BUPIVACAINE HYDROCHLORIDE:Supplementation therapy:0.82), (CABOZANTINIB S-MALATE:Supplementation therapy:0.94), (CAJUPUT OIL\EUCALYPTUS OILPEPPERMINT OIL:Supplementation therapy:0.07), (CALCIUM BICARBONATE:Supplementation therapy:0.14), (CALCIUM PANTOTHENATECUPRIC SULFATECYANOCOBALAMINFERROUS ASPARTO GLYCINATEFERROUS FUMARATEFOLIC ACIDMAGNESIUM SULFATENIACINAMIDEPYRIDOXINE HYDROCHLORIDE\RIBOFLAVIN\SODIUM ASCORBATETHIAMINE MONONITRATE\ZINC SULFATE:Supplementation therapy:0.07), (CALCIUMCHOLECALCIFEROLCOPPERMAGNESIUMMANGANESEZI NC:Supplementation therapy:0.15), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:Supplementation therapy:0.08), (CARBIMAZOLE:Supplementation therapy:1.17), (CARBOMER:Supplementation therapy:0.03), (CARBOXYMETHYLCELLULOSE SODIUMHYPROMELLOSES:Supplementation therapy:0.04), (CARMUSTINE:Supplementation therapy:0.56), (CEFOTAXIMECEFOTAXIME SODIUM:Supplementation therapy:0.28), (CEFTAZIDIME SODIUM:Supplementation therapy:0.01), (CELIPROLOL:Supplementation therapy:0.05), (CERIVASTATIN SODIUM:Supplementation therapy:1.04), (CHLOPHEDIANOL HYDROCHLORIDEDEXBROMPHENIRAMINE MALEATEPHENYLEPHRINE HYDROCHLORIDE:Supplementation therapy:0.12), (CHLORDIAZEPOXIDECLIDINIUM:Supplementation therapy:0.27), (CHLORMEZANONE:Supplementation therapy:0.08), (CHLORPROTHIXENE:Supplementation therapy:0.04), (CHOLECALCIFEROLGENISTEINZINC GLYCINATE:Supplementation therapy:0.27), (CICLOPIROX OLAMINE:Supplementation therapy:0.99), (CLIKSTAR:Supplementation therapy:0.82), (CLOCORTOLONE PIVALATE:Supplementation therapy:0.13), (CLOFAZIMINE:Supplementation therapy:0.11), (CLOMIPRAMINE HYDROCHLORIDE:Supplementation therapy:0.93), (CLOSTRIDIUM BUTYRICUM SPORES STRAIN M-55:Supplementation therapy:0.29), (CLOXAZOLAM:Supplementation therapy:0.36), (COAGULATION FACTOR IX HUMANCOAGULATION FACTOR VII HUMANCOAGULATION FACTOR X HUMANPROTEIN CPROTEIN S HUMANPROTHROMBIN:Supplementation therapy:0.01), (COAGULATION FACTOR VIIA RECOMBINANT HUMAN:Supplementation therapy:0.06), (COBICISTATDARUNAVIR ETHANOLATE:Supplementation therapy:0.45), (COBICISTATELVITEGRAVIREMTRICITABINETENOFOVIR DISOPROXIL FUMARATE:Supplementation therapy:0.59), (CODEINE PHOSPHATEPROMETHAZINE:Supplementation therapy:0.61), (CODEINE PHOSPHATEPROMETHAZINE HYDROCHLORIDE:Supplementation therapy:0.33), (CODEINE SULFATE:Supplementation therapy:0.46), (COLISTIMETHATE SODIUM:Supplementation therapy:0.74), (COLISTIN:Supplementation therapy:0.79), (CREATININE:Supplementation therapy:0.01), (CROFELEMER:Supplementation therapy:0.05), (CROTALIDAE POLYVALENT IMMUNE FAB (OVINE):Supplementation therapy:0.07), (DABRAFENIB MESYLATE:Supplementation therapy:0.88), (DANTROLENE SODIUM:Supplementation therapy:1.24), (DARUNAVIRRITONAVIR:Supplementation therapy:0.14), (DESFLURANE:Supplementation therapy:0.54), (DEXTRAN SULFATE SODIUM SALT:Supplementation therapy:0.11), (DICHLORPHENAMIDE:Supplementation therapy:0.23), (DIENOGEST:Supplementation therapy:0.49), (DIETARY SUPPLEMENTFLAVOCOXID:Supplementation therapy:0.05), (DIHYDROCODEINE:Supplementation therapy:0.11), (DIMETHYL SULFOXIDE:Supplementation therapy:0.61), (DIPHTHERIA AND TETANUS TOXOIDS AND ACELLULAR PERTUSSIS VACCINE ADSORBED:Supplementation therapy:0.52), (DIPYRONEPROMETHAZINE HYDROCHLORIDE:Supplementation therapy:1.59), (DOCUSATE SODIUMSENNOSIDE ASENNOSIDE B:Supplementation therapy:0.06), (DOLASETRON MESYLATE:Supplementation therapy:1.58), (DOXYCYCLINE HYDROCHLORIDE:Supplementation therapy:0.14), (DROSPIRENONEESTRADIOL:Supplementation therapy:0.1), (ECALLANTIDE:Supplementation therapy:0.86), (ELBASVIRGRAZOPREVIR:Supplementation therapy:1.07), (EMPAGLIFLOZINMETFORMIN HYDROCHLORIDE:Supplementation therapy:0.37), (EMTRICITABINERILPIVIRINE HYDROCHLORIDETENOFOVIR DISOPROXIL FUMARATE:Supplementation therapy:0.82), (ENALAPRIL MALEATELERCANIDIPINE HYDROCHLORIDE:Supplementation therapy:0.59), (EPHEDRINE HYDROCHLORIDEGUAIFENESIN:Supplementation therapy:0.8), (EPINASTINE HYDROCHLORIDE:Supplementation therapy:0.06), (EPIRUBICIN:Supplementation therapy:0.95), (ERIBULIN:Supplementation therapy:0.59), (ESTRADIOL BENZOATE:Supplementation therapy:0.87), (ESTRADIOLLEVONORGESTREL:Supplementation therapy:0.68), (ETHINYL ESTRADIOLNORELGESTROMIN:Supplementation therapy:0.26), (ETHOSUXIMIDE:Supplementation therapy:0.2), (ETIMICIN SULFATE:Supplementation therapy:1.26), (ETOPOSIDE PHOSPHATE:Supplementation therapy:0.18), (FELBAMATE:Supplementation therapy:1.21), (FENOPROFEN CALCIUM:Supplementation therapy:0.47), (FENOTEROL HYDROBROMIDE:Supplementation therapy:1.04), (FERRIC HYDROXIDE:Supplementation therapy:0.3), (FERROUS BISGLYCINATE:Supplementation therapy:0.97), (FLUCYTOSINE:Supplementation therapy:0.61), (FLUNARIZINE HYDROCHLORIDE:Supplementation therapy:0.47), (FLUOXETINE HYDROCHLORIDEOLANZAPINE:Supplementation therapy:0.45), (FLUTAMIDE:Supplementation therapy:0.23), (FORMOTEROL FUMARATE\MOMETASONE FUROATE:Supplementation therapy:1.79), (FOSFOMYCIN:Supplementation therapy:0.27), (GENTIAN VIOLET:Supplementation therapy:0.04), (GIMERACILOTERACILTEGAFUR:Supplementation therapy:0.63), (GLATIRAMER:Supplementation therapy:0.6), (GLIPIZIDEMETFORMIN HYDROCHLORIDE:Supplementation therapy:1.25), (GLUCONATE:Supplementation therapy:0.32), (GLYCOPYRROLATEINDACATEROL MALEATE:Supplementation therapy:0.93), (GOLD:Supplementation therapy:0.16), (GUAIFENESINPHENYLEPHRINE HYDROCHLORIDE:Supplementation therapy:0.47), (GUSPERIMUS TRIHYDROCHLORIDE:Supplementation therapy:0.85), (HALCINONIDE:Supplementation therapy:0.3), (HALOPERIDOL DECANOATE:Supplementation therapy:0.27), (HEMIN:Supplementation therapy:0.07), (HEMP:Supplementation therapy:0.31), (HERBALSHOMEOPATHICSGOLDENSEAL:Supplementation therapy:0.07), (HIGH DENSITY POLYETHYLENE:Supplementation therapy:0.72), (HYDRALAZINE HYDROCHLORIDEISOSORBIDE DINITRATE:Supplementation therapy:0.06), (HYDRAZINE SULFATE:Supplementation therapy:0.34), (HYDROCHLORIC ACID:Supplementation therapy:0.25), (HYDROCORTISONE VALERATE:Supplementation therapy:0.1), (IBRITUMOMAB TIUXETAN:Supplementation therapy:0.75), (INDACATEROL:Supplementation therapy:1.32), (INDACATEROL MALEATE:Supplementation therapy:0.22), (INSULIN PORK\INSULIN PURIFIED PORK:Supplementation therapy:0.87), (INTERFERON ALFA-2A:Supplementation therapy:1.64), (IRON DEXTRAN:Supplementation therapy:0.05), (ITRACONAZOLE\SODIUM CHLORIDE:Supplementation therapy:0.74), (IXABEPILONE:Supplementation therapy:0.1), (IXAZOMIB CITRATE:Supplementation therapy:0.02), (IXEKIZUMAB:Supplementation therapy:0.15), (LACTOPEROXIDASE BOVINELYSOZYME HYDROCHLORIDE:Supplementation therapy:0.02), (LANOLINMINERAL OILPETROLATUM:Supplementation therapy:0.33), (LANREOTIDE:Supplementation therapy:0.78), (LATANOPROSTTIMOLOL:Supplementation therapy:0.68), (LEVAMLODIPINE:Supplementation therapy:0.06), (LEVOBUNOLOL:Supplementation therapy:0.26), (LEVOMEFOLATE CALCIUM\METHYLCOBALAMINPYRIDOXAL 5-PHOSPHATE:Supplementation therapy:0.04), (LEVOMEPROMAZINE:Supplementation therapy:0.02), (LEVOTHYROXINE SODIUMPOTASSIUM IODIDE:Supplementation therapy:0.38), (LOMUSTINE:Supplementation therapy:0.03), (LUTETIUM LU-177:Supplementation therapy:0.72), (LYCOPENE:Supplementation therapy:0.48), (MAGNESIUM ASPARTATE HYDROCHLORIDE:Supplementation therapy:0.06), (MAGNESIUM SULFATE\POTASSIUM SULFATE\SODIUM SULFATE:Supplementation therapy:0.34), (MECLIZINE MONOHYDROCHLORIDENIACIN:Supplementation therapy:0.5), (MECLOZINE DIHYDROCHLORIDE:Supplementation therapy:0.59), (MELPERONE:Supplementation therapy:0.09), (MENTHOLZINC OXIDE:Supplementation therapy:0.86), (MESTRANOLNORETHINDRONE:Supplementation therapy:0.93), (METFORMIN HYDROCHLORIDEROSIGLITAZONE MALEATE:Supplementation therapy:0.24), (METFORMIN HYDROCHLORIDESAXAGLIPTIN:Supplementation therapy:0.22), (METFORMIN HYDROCHLORIDESAXAGLIPTIN HYDROCHLORIDE:Supplementation therapy:0.94), (METHENAMINE MANDELATE:Supplementation therapy:1.32), (METHENOLONE ENANTHATE:Supplementation therapy:0.64), (METHYLENE BLUE:Supplementation therapy:0.26), (METILDIGOXIN:Supplementation therapy:0.93), (MIGLUSTAT:Supplementation therapy:0.85), (MITOXANTRONE HYDROCHLORIDE:Supplementation therapy:0.84), (MOEXIPRIL HYDROCHLORIDE:Supplementation therapy:0.98), (MORNIFLUMATE:Supplementation therapy:0.26), (NAFTIFINE HYDROCHLORIDE:Supplementation therapy:0.5), (NALBUPHINE HYDROCHLORIDE:Supplementation therapy:0.68), (NALOXONE:Supplementation therapy:1.32), (NALOXONE HYDROCHLORIDETILIDINE HYDROCHLORIDE:Supplementation therapy:1.23), (NEFAZODONE HYDROCHLORIDE:Supplementation therapy:0.79), (NOREPINEPHRINE:Supplementation therapy:1.43), (NUSINERSEN:Supplementation therapy:0.65), (NYSTATINTRIAMCINOLONE ACETONIDE:Supplementation therapy:0.19), (OXYBUTYNINOXYBUTYNIN CHLORIDE:Supplementation therapy:1.52), (OXYQUINOLINE SULFATE\SODIUM LAURYL SULFATE:Supplementation therapy:0.05), (PANITUMUMAB:Supplementation therapy:0.64), (PARSLEY:Supplementation therapy:0.2), (PATIROMER:Supplementation therapy:1.96), (PEGINTERFERON ALFA-2A OR PEGINTERFERON ALFA-2B:Supplementation therapy:0.23), (PENTAZOCINE LACTATE:Supplementation therapy:0.27), (PENTOSTATIN:Supplementation therapy:0.82), (PERTUZUMAB:Supplementation therapy:0.93), (PHENELZINE SULFATE:Supplementation therapy:0.3), (PHENPROCOUMON:Supplementation therapy:1.22), (PIRFENIDONE:Supplementation therapy:0.81), (PIZOTYLINE:Supplementation therapy:0.06), (PODOFILOX:Supplementation therapy:0.09), (POLYMYXIN:Supplementation therapy:0.88), (POLYMYXIN B SULFATETRIMETHOPRIM SULFATE:Supplementation therapy:0.74), (POLYVINYL ALCOHOL:Supplementation therapy:0.39), (PORFIMER SODIUM:Supplementation therapy:0.21), (POTASSIUM BITARTRATE:Supplementation therapy:0.61), (PREDNISOLONE ACETATESULFACETAMIDE SODIUM:Supplementation therapy:0.06), (PRISTINAMYCIN:Supplementation therapy:0.03), (PROCAINE HYDROCHLORIDE:Supplementation therapy:0.04), (PROPATYL NITRATE:Supplementation therapy:0.37), (PROTRIPTYLINE HYDROCHLORIDE:Supplementation therapy:1.97), (PSORALEN:Supplementation therapy:0.08), (QUINACRINE:Supplementation therapy:1.5), (QUINIDINE GLUCONATE:Supplementation therapy:0.49), (RALTEGRAVIR:Supplementation therapy:0.53), (REMIFENTANIL HYDROCHLORIDE:Supplementation therapy:0.2), (ROFECOXIB:Supplementation therapy:0.59), (ROPIVACAINE:Supplementation therapy:0.08), (RUPATADINE FUMARATE:Supplementation therapy:0), (SARGRAMOSTIM:Supplementation therapy:0.03), (SEBELIPASE ALFA:Supplementation therapy:0.46), (SELEXIPAG:Supplementation therapy:1.09), (SIBUTRAMINE HYDROCHLORIDE:Supplementation therapy:1.13), (SIMEPREVIR:Supplementation therapy:0.37), (SODIUM CITRATE:Supplementation therapy:1.14), (SODIUM PHOSPHATE, DIBASIC:Supplementation therapy:0.1), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Supplementation therapy:0.46), (SOMATOSTATIN:Supplementation therapy:0.27), (SOMATREM:Supplementation therapy:0.43), (SORBITOL:Supplementation therapy:0.56), (SUGAMMADEX:Supplementation therapy:0.02), (SULTOPRIDE HYDROCHLORIDE:Supplementation therapy:0.06), (TALIMOGENE LAHERPAREPVEC:Supplementation therapy:0.06), (TAZAROTENE:Supplementation therapy:0.45), (TEDUGLUTIDE\WATER:Supplementation therapy:2.12), (TEGASEROD:Supplementation therapy:0.08), (TELAPREVIR:Supplementation therapy:0.45), (TELOTRISTAT ETHYL:Supplementation therapy:1.25), (TENOFOVIR:Supplementation therapy:0.28), (TERCONAZOLE:Supplementation therapy:1.29), (TESAMORELIN:Supplementation therapy:0.9), (TESTOSTERONE UNDECANOATE:Supplementation therapy:1.49), (THIOPENTAL:Supplementation therapy:0.49), (THIOPENTAL SODIUM:Supplementation therapy:0.16), (TIBOLONE:Supplementation therapy:1.05), (TICLOPIDINE:Supplementation therapy:0.07), (TILIDINE:Supplementation therapy:0.01), (TIMOLOL MALEATETRAVOPROST:Supplementation therapy:0.89), (TIOPRONIN:Supplementation therapy:0.49), (TOLMETIN:Supplementation therapy:0.35), (TOLMETIN SODIUM:Supplementation therapy:0.25), (TRABECTEDIN:Supplementation therapy:0.17), (TRAMETINIB DIMETHYL SULFOXIDE:Supplementation therapy:0.94), (TRANYLCYPROMINE:Supplementation therapy:0.37), (TRANYLCYPROMINE SULFATE:Supplementation therapy:0.65), (TRICHLORMETHIAZIDE:Supplementation therapy:0.1), (TRIMETHADIONE:Supplementation therapy:0.05), (TRIMETHOPRIM SULFATE:Supplementation therapy:0.11), (TRIMIPRAMINE MALEATE:Supplementation therapy:0.77), (TRIPTORELIN PAMOATE:Supplementation therapy:1.04), (TRYPTOPHAN:Supplementation therapy:0.56), (URAPIDIL:Supplementation therapy:0.91), (VALDECOXIB:Supplementation therapy:0.81), (VALRUBICIN:Supplementation therapy:0.22), (VEMURAFENIB:Supplementation therapy:1.41), (VINORELBINEVINORELBINE TARTRATE:Supplementation therapy:0.59), (VITAMIN A PALMITATE:Supplementation therapy:0.48), (VORAPAXAR SULFATE:Supplementation therapy:0.07), (WARFARIN POTASSIUM:Supplementation therapy:0.19), (ZIV-AFLIBERCEPT:Supplementation therapy:0.41), (AMOXICILLINCLAVULANATE POTASSIUM:Supportive care:0.3), (BENDAMUSTINE:Supportive care:0.06), (CALCIUM ACETATE:Supportive care:0.02), (CANDESARTAN CILEXETIL:Supportive care:0.44), (CANDESARTAN CILEXETILHYDROCHLOROTHIAZIDE:Supportive care:0.15), (CERTOLIZUMAB PEGOL:Supportive care:0.85), (CETUXIMAB:Supportive care:0.89), (CIMETIDINE:Supportive care:0.11), (CLOZAPINE:Supportive care:0.14), (CORTISONEHYDROCORTISONE:Supportive care:0.26), (CRIZOTINIB:Supportive care:0.36), (DASATINIB:Supportive care:0.82), (DICLOFENAC SODIUM:Supportive care:0.33), (DOCUSATE:Supportive care:0.02), (DRONABINOL:Supportive care:0.07), (ERGOCALCIFEROL:Supportive care:0.5), (ESCITALOPRAM OXALATE:Supportive care:0.58), (ESZOPICLONE:Supportive care:0.12), (EVOLOCUMAB:Supportive care:0.18), (FENOFIBRATE:Supportive care:0.03), (FERROUS SULFATEFERROUS SULFATE, DRIED:Supportive care:1.07), (FLECAINIDE:Supportive care:0.1), (GIMERACILOTERACILTEGAFUR:Supportive care:0), (HYDROCHLOROTHIAZIDELISINOPRIL:Supportive care:0.2), (IDELALISIB:Supportive care:0.14), (ISOSORBIDE MONONITRATE:Supportive care:0.24), (LAPATINIB DITOSYLATE:Supportive care:0.06), (LERCANIDIPINE:Supportive care:0.04), (MOXIFLOXACIN:Supportive care:0.25), (NILOTINIB:Supportive care:1.4), (OXCARBAZEPINE:Supportive care:0.45), (PHENOBARBITAL:Supportive care:0.01), (PONATINIB:Supportive care:0.51), (PRAVASTATIN:Supportive care:0.57), (RANOLAZINE:Supportive care:0.1), (REGORAFENIB:Supportive care:0.5), (SACUBITRILVALSARTAN:Supportive care:0.26), (SECUKINUMAB:Supportive care:0.03), (SOTALOL:Supportive care:0.17), (TAMSULOSIN HYDROCHLORIDE:Supportive care:0.21), (TELMISARTAN:Supportive care:0.34), (TIMOLOL:Supportive care:0.09), (TINZAPARIN SODIUM:Supportive care:0.18), (TOFACITINIB CITRATE:Supportive care:0.07), (TRIAMCINOLONE ACETONIDE:Supportive care:0.25), (VERAPAMIL HYDROCHLORIDE:Supportive care:0.63), (VINORELBINE TARTRATE:Supportive care:0.1), (ACARBOSE:Supraventricular tachycardia:0.08), (ACETAMINOPHENCODEINE PHOSPHATE:Supraventricular tachycardia:1.05), (ALCOHOL:Supraventricular tachycardia:0.03), (AMITRIPTYLINE HYDROCHLORIDE:Supraventricular tachycardia:0.13), (AMOXICILLIN:Supraventricular tachycardia:0.8), (BISACODYL OR DOCUSATE SODIUM:Supraventricular tachycardia:0.57), (CALCIUM ACETATE:Supraventricular tachycardia:0.46), (CANAGLIFLOZINMETFORMIN HYDROCHLORIDE:Supraventricular tachycardia:0.13), (CANDESARTAN:Supraventricular tachycardia:1.25), (CANDESARTAN CILEXETILHYDROCHLOROTHIAZIDE:Supraventricular tachycardia:0.8), (CARFILZOMIB:Supraventricular tachycardia:0.62), (CARVEDILOL PHOSPHATE:Supraventricular tachycardia:0.4), (CERTOLIZUMAB PEGOL:Supraventricular tachycardia:1.09), (CIMETIDINE:Supraventricular tachycardia:0.31), (CLINDAMYCINCLINDAMYCIN PHOSPHATE:Supraventricular tachycardia:0.69), (CLONIDINE:Supraventricular tachycardia:0.74), (CLOZAPINE:Supraventricular tachycardia:0.27), (CORTISONEHYDROCORTISONE:Supraventricular tachycardia:0.21), (DACLIZUMAB:Supraventricular tachycardia:0.21), (DALFAMPRIDINE:Supraventricular tachycardia:1.22), (DAPAGLIFLOZIN PROPANEDIOL:Supraventricular tachycardia:0.24), (DIETARY SUPPLEMENT:Supraventricular tachycardia:0.19), (DOXAZOSIN MESYLATE:Supraventricular tachycardia:0.4), (EDOXABAN TOSYLATE:Supraventricular tachycardia:0.37), (ELETRIPTAN HYDROBROMIDE:Supraventricular tachycardia:0.51), (EVEROLIMUS:Supraventricular tachycardia:0.76), (FINGOLIMOD HYDROCHLORIDE:Supraventricular tachycardia:0.49), (FORMOTEROL FUMARATE DIHYDRATE\MOMETASONE FUROATE:Supraventricular tachycardia:0.11), (GLYCOPYRRONIUMANDACATEROL:Supraventricular tachycardia:0.03), (HYDROCHLOROTHIAZIDEVALSARTAN:Supraventricular tachycardia:0.22), (HYDROCODONE:Supraventricular tachycardia:0.8), (HYDROXYZINE PAMOATE:Supraventricular tachycardia:0.65), (HYOSCYAMINE:Supraventricular tachycardia:0.01), (ILOPROST:Supraventricular tachycardia:1.22), (IMIPRAMINE HYDROCHLORIDE:Supraventricular tachycardia:0.04), (LABETALOLLABETALOL HYDROCHLORIDE:Supraventricular tachycardia:0.18), (LERCANIDIPINE HYDROCHLORIDE:Supraventricular tachycardia:0.17), (LINACLOTIDE:Supraventricular tachycardia:0.08), (LINAGLIPTIN:Supraventricular tachycardia:0.65), (LITHIUM CARBONATE:Supraventricular tachycardia:0.48), (LOVASTATIN:Supraventricular tachycardia:1.55), (MELATONIN:Supraventricular tachycardia:1.13), (METAXALONE:Supraventricular tachycardia:0.73), (NALOXONE HYDROCHLORIDEOXYCODONE HYDROCHLORIDE:Supraventricular tachycardia:0.21), (NITROFURANTOIN:Supraventricular tachycardia:0.64), (NYSTATIN:Supraventricular tachycardia:0.51), (OLANZAPINE:Supraventricular tachycardia:0.47), (PANTOPRAZOLE MAGNESIUM:Supraventricular tachycardia:0.47), (PAROXETINE:Supraventricular tachycardia:0.52), (PAZOPANIB HYDROCHLORIDE:Supraventricular tachycardia:0.18), (PENICILLIN:Supraventricular tachycardia:0.24), (PERINDOPRIL:Supraventricular tachycardia:0.28), (PONATINIB:Supraventricular tachycardia:0.56), (PYRIDOXINE HYDROCHLORIDE:Supraventricular tachycardia:0.02), (ROFLUMILAST:Supraventricular tachycardia:0.01), (SCOPOLAMINE:Supraventricular tachycardia:0.05), (SECUKINUMAB:Supraventricular tachycardia:0.67), (SELEXIPAG:Supraventricular tachycardia:0.54), (SULFAMETHOXAZOLE:Supraventricular tachycardia:0.03), (SUMATRIPTAN:Supraventricular tachycardia:0.2), (SUMATRIPTANSUMATRIPTAN SUCCINATE:Supraventricular tachycardia:0.41), (TETRACYCLINE:Supraventricular tachycardia:0.16), (TRIAMCINOLONE ACETONIDE:Supraventricular tachycardia:0.36), (TRIAMTERENE:Supraventricular tachycardia:0.39), (UMECLIDINIUM BROMIDEVILANTEROL TRIFENATATE:Supraventricular tachycardia:0.49), (VARENICLINE TARTRATE:Supraventricular tachycardia:0.97), (VINCRISTINE SULFATE:Supraventricular tachycardia:0.13), (.ALPHA.1-PROTEINASE INHIBITOR HUMAN:Surgery:0.02), (ACARBOSE:Surgery:0.15), (ACETAMINOPHENBUTALBITALCA FFEINE:Surgery:1.29), (ADO-TRASTUZUMAB EMTANSINE:Surgery:0.07), (ALCOHOL:Surgery:0.24), (ALENDRONIC ACID:Surgery:0.22), (AMIODARONE HYDROCHLORIDE:Surgery:1.11), (AMLODIPINEVALSARTAN:Surgery:0.18), (AMPICILL IN:Surgery:0.53), (AZITHROMYCIN DIHYDRATE:Surgery:0.69), (BENZONATATE:Surgery:0.29), (BISACODYL OR DOCUSATE SODIUM:Surgery:1.07), (BORTEZOMIB:Surgery:0.15), (BROMAZEPAM:Surgery:0.23), (BUD ESONIDEFORMOTEROL FUMARATE:Surgery:0.23), (BUPIVACAINEEPINEPHRINE:Surgery:0.45), (BUPRENORPHI NE HYDROCHLORIDENALOXONE HYDROCHLORIDE:Surgery:0.53), (CALCITONIN SALMON:Surgery:0.66), (CALCIUM ACETATE:Surgery:0.69), (CALCIUM CARBONATECHOLECALCIFEROL:Surgery:1.36), (CANAGLIFLOZINMETFORMIN HYDROCHLORIDE:Surgery:0.02), (CANAKINUMAB:Surgery:0.06), (CARFILZOMIB:Surger y:0.64), (CETIRIZINE HYDROCHLORIDEPSEUDOEPHEDRINE HYDROCHLORIDE:Surgery:0.01), (CILASTATIN SODIUMIMIPENEM:Surgery:0.1), (CILOSTAZOL:Surgery:0.2), (CLOZAPINE:Surgery:0.8), (CORTICOTROPIN:Surgery:0.04), (DACLIZUMAB:Surgery:0.05), (DALFAMPRIDINE:Surger y:0.89), (DAPAGLIFLOZIN PROPANEDIOL:Surgery:0.07), (DASATINIB:Surgery:0.73), (DEXLANSOPRAZOLE:Surgery: 1.71), (DIGITOXIN:Surgery:0.01), (DIMENHYDRINATE:Surgery:0.53), (DOBUTAMINE HYDROCHLORIDE:Surgery:0.1), (DOXEPIN:Surgery:0.07), (DRONABINOL:Surgery:0.55), (E MPAGLIFLOZINLINAGLIPTIN:Surgery:0.06), (EPLERENONE:Surgery:0.1), (ESTROGENS: Surgery:0.01), (EVEROLIMUS:Surgery:1.03), (FENOFIBRIC ACID:Surgery:0.1), (FINGOLIMOD HYDROCHLORIDE:Surgery:0.44), (FLUDROCORTISONE ACETATE:Surgery:0.18), (FLUTICASONE:Surgery:0.61), (FLUTICASONE FUROATESALMETEROL XINAFOATE:Surgery:0.39), (FLUTICASONE FUROATEVILANTEROL TRIFENATATE:Surgery:0.45), (FOSPHENYTOINFOSPHENYTOIN SODIUM:Surgery:0.78), (FUSIDIC ACID:Surgery:0.2), (GLIPIZIDE:Surgery:0.8), (GLYCOPYRRONIUMlNDACATEROL:Surger y:0.18), (HYALURONATE SODIUM:Surgery:0.27), (HYDROCHLOROTHIAZIDEIRBESARTAN:Surgery:0.39), (HYDR OCHLOROTHIAZIDELOSARTAN POTASSIUM:Surgery:0.48), (HYDROCHLOROTHIAZIDETELMISARTAN:Surgery:0.19), (H YDROCORTISONE ACETATE:Surgery:0.02), (HYDROXYCHLOROQUINE DIPHOSPHATE:Surgery:0.01), (INDAPAMIDEPERINDOPRIL:Surgery:0.23), (IPRATROPIU M:Surgery:0.95), (ISOFLURANE:Surgery:1.56), (LEDIPASVIRSOFOSBUVIR:Surgery:0.61), (LIDOCAINEPRILOCAINE:Surgery:0.3), (LISDEXAMFETAMINE DIMESYLATE:Surgery:0.52), (LOMITAPIDE MESYLATE:Surgery:0), (METHENAMINE HIPPURATE:Surgery:0.7), (METHYLPREDNISOLONE ACETATE:Surgery:1.03), (MIDODRINE:Surgery:0.11), (MINOCYCLINE HYDROCHLORIDE:Surgery:0.07), (MINOCYCLINEMINOCYCLINE HYDROCHLORIDE:Surgery:0.59), (NADOLOL:Surgery:0.32), (NALOXONE HYDROCHLORIDEOXYCODONE HYDROCHLORIDE:Surgery:0.77), (NEBIVOLOL:Surgery:0.63), (NILOTINIB:Surgery:1), (NI NTEDANIB:Surgery:0.09), (NOREPINEPHRINE BITARTRATE:Surgery:0.26), (OXAZEPAM:Surgery:0.02), (PANCURONIUM:Surgery:0.03), (P ANTOPRAZOLE:Surgery:1.95), (PEGFILGRASTIM:Surgery:0.14), (PEGINTERFERON BETA-1A:Surgery:0.23), (PENTOBARBITAL:Surgery:0.18), (PITAVASTATIN CALCIUM:Surgery:0.21), (PONATINIB:Surgery:0.33), (PROPRANOLOLPROPRANOLOL HYDROCHLORIDE:Surgery:0.24), (PYRIDOXINE:Surgery:0.12), (PYRIDOXINE HYDROCHLORIDE:Surgery:0.39), (ROFLUMILAST:Surgery:0.32), (SACUBITRILVALSAR TAN:Surgery:0.68), (SECUKINUMAB:Surgery:0.61), (SODIUM CITRATE:Surgery:0.04), (SUMATRIPTANSUMATRIPTAN SUCCINATE:Surgery:1.09), (TERBINAFINE HYDROCHLORIDE:Surgery:0.09), (TERBUTALINE:Surgery:0.19), (TERBUTALINE SULFATE:Surgery:0.31), (THIAMYLAL:Surgery:0.02), (THIAMYLAL SODIUM:Surgery:0.73), (THYROID, PORCINE:Surgery:0.2), (TIMOLOL:Surgery:0.71), (TOCILIZUMAB:Surgery:0.5), (UMECLIDI NIUM BROMIDEVILANTEROL TRIFENATATE:Surgery:0.66), (VALGANCICLOVIR HYDROCHLORIDE:Surgery:0.35), (ZONISAMIDE:Surgery:0.16), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Swelling:0.05), (ACETAMINOPHENPROPOXYPHENE NAPSYLATE:Swelling:0.06), (ACETAMINOPHENTRAMADOL:Swelling:0.12), (ADO-TRASTUZUMAB EMTANSINE:Swelling:0.28), (ALBIGLUTIDE:Swelling:0.51), (ALENDRONIC ACID:Swelling:0.59), (ALFACALCIDOL:Swelling:0.32), (ALISKIREN HEMIFUMARATE:Swelling:0.51), (AMANTADINE:Swelling:0.34), (AMINO ACIDSDEXTROSEELECTROLYTES NOS:Swelling:0.1), (AMLODIPINE BESYLATEHYDROCHLOROTHIAZIDEOLMESARTAN MEDOXOMIL:Swelling:0.35), (AMLODIPINEVALSARTAN:Swelling:0.33), (AMPHOTERICIN B:Swelling:0.25), (ANAKINRA:Swelling:0.06), (AZELASTINE:Swelling:0.25), (BETAMETHA SONE DIPROPIONATECALCIPOTRIENECALCIPOTRIENE HYDRATE:Swelling:0.53), (BUPRENORPHINE HYDROCHLORIDE:Swelling:0.54), (CALCIPOTRIENE:Swelling:0.14), (CALCIUMMAGNE SIUM\ZINC:Swelling:0.59), (CANAGLIFLOZINMETFORMIN HYDROCHLORIDE:Swelling:0.85), (CANDESARTAN CILEXETILHYDROCHLOROTHIAZIDE:Swelling:1.28), (CARVEDILOL PHOSPHATE:Swelling:1.77), (CEFAZOLIN:Swelling:0.1), (CHLORDIAZEPOXIDE HYDROCHLORIDECLIDINIUM BROMIDE:Swelling:0.23), (CIMETIDINE:Swelling:1.19), (CIPROFIBRATE:Swelling:0.02), (C YPROHEPTADINE:Swelling:0.13), (DABIGATRAN:Swelling:0.04), (DACLIZUMAB:Swelling: 1.23), (DALTEPARIN:Swelling:0.14), (DAPAGLIFLOZIN:Swelling:0.02), (DASATINIB:Swelli ng:1.35), (DESONIDE:Swelling:0.44), (DOCETAXEL:Swelling:0.34), (EDOXABAN TOSYLATE:Swelling:0.75), (EMPAGLIFLOZINLINAGLIPTIN:Swelling:0.86), (ESTRONES US SCROFA OVARY:Swelling:0.06), (ESZOPICLONE:Swelling:1.66), (FLUOCINONIDE:Swelling:0.67), (F LUVOXAMINE MALEATE:Swelling:0.27), (FORMOTEROL FUMARATE:Swelling:0.85), (FORMOTEROL FUMARATE\MOMETASONE FUROATE:Swelling:0.53), (FUSIDIC ACID:Swelling:0.36), (GLICLAZIDE:Swelling:0.67), (GLYCOPYRROLATE:Swelling:0.38), (G LYCOPYRRONIUMINDACATEROL:Swelling:0.5), (GUANFACINE HYDROCHLORIDE:Swelling:0.14), (HYDROCHLOROTHIAZIDETELMISARTAN:Swelling: 0.39), (HYDROCORTISONE ACETATE:Swelling:0.02), (IMATINIB MESYLATE:Swelling:1.04), (IMIPRAMINE HYDROCHLORIDE:Swelling:1.45), (INDAPAMIDEPERINDOPRIL:Swelling:0.44), (INFLIXI MAB-DYYB:Swelling:0.26), (IODINE:Swelling:0.17), (LABETALOLLABETALOL HYDROCHLORIDE:Swelling:1.45), (LACTASE:Swelling:0.59), (LAPATINIB DITOSYLATE:Swelling:0.12), (LEDIPASVIRSOFOSBUVIR:Swelling:1.27), (LERCANIDIPI NE:Swelling:0.71), (LITHIUM CARBONATE:Swelling:1.12), (MEGESTROL ACETATE:Swelling:0.5), (MEPOLIZUMAB:Swelling:0.24), (METHENAMINE HIPPURATE:Swelling:1.14), (MIDODRINE HYDROCHLORIDE:Swelling:0.06), (MOXIFLOXACIN:Swelling:0.4), (NALTREXONE:Swelli ng:0.31), (NATEGLINIDE:Swelling:0.45), (NILOTINIB:Swelling:1.16), (OXYBUTYNINOXY BUTYNIN CHLORIDE:Swelling:0.09), (PAMIDRONATE DISODIUM:Swelling:0.21), (PARICALCITOL:Swelling:0.14), (PAZOPANIB HYDROCHLORIDE:Swelling:0.7), (PEMETREXED DISODIUM:Swelling:0.68), (PERINDOPRIL:Swelling:0.67), (PIMECROLIMUS:Swelling:0.2), (PIRFENIDONE:Swelling:0.27), (PRAZOSIN:Swelling:0.47), (PREDNISOLONEPREDNISOL ONE ACETATEPREDNISOLONE SODIUM SUCCINATE:Swelling:0.04), (PROPAFENONE HYDROCHLORIDE:Swelling:0.63), (PROTRIPTYLINE HYDROCHLORIDE:Swelling:0.07), (RALOXIFENE HYDROCHLORIDE:Swelling:1.07), (RETINOL:Swelling:0.05), (SITAGLIPTIN:Swelling:0.14), (SULINDAC:Swelling:0.2), (TERBUTALINE SULFATE:Swelling:0.93), (THALIDOMIDE:Swelling:0.74), (TIMOLOL MALEATE:Swelling:0.6), (UMECLIDINIUM BROMIDE:Swelling:0.44), (ZILEUTON:Swelling:0.18), (ZINC:Swelling:1.01), (ZOLMITRIPTA N:Swelling:0.08), (ANASTROZOLE:Syncope:0.17), (BISACODYL OR DOCUSATE SODIUM:Syncope:0.22), (BUDESONIDE:Syncope:0.88), (CALCIUM CARBONATECHOLECALCIFEROL:Syncope:0.34), (CANDESARTAN:Syncope:0.27), (CER TOLIZUMAB PEGOL:Syncope:0.43), (CLINDAMYCINCLINDAMYCIN PHOSPHATE:Syncope:0.23), (CLOTRIMAZOLE:Syncope:0.11), (DACLIZUMAB:Syncope:0.07), (DEVICE:Syncope:0.01), (DICLOFENAC:Syncope:0.31), (ELETRIPTAN HYDROBROMIDE:Syncope:0.15), (ENALAPRIL MALEATE:Syncope:0.2), (ESOMEPRAZOLE:Syncope:0.06), (EVOLOCUMAB:Syncope:0.57), (FERROUS SULFATEFERROUS SULFATE, DRIED:Syncope:0.57), (HYDROCHLOROTHIAZIDELISINOPRIL:Syncope:0.37), (HYOSCY AMINE:Syncope:0.04), (IBRUTINIB:Syncope:0.52), (ISOSORBIDE:Syncope:0.14), (LACOSA MIDE:Syncope:0.43), (LATANOPROST:Syncope:0.01), (METAXALONE:Syncope:0.55), (MO DAFINIL:Syncope:0.34), (NAPROXEN SODIUM:Syncope:0.07), (OLANZAPINE:Syncope:0.35), (OMEGA-3-ACID ETHYL ESTERS:Syncope:0.31), (PRAMIPEXOLE DIHYDROCHLORIDE:Syncope:0.68), (PRIMIDONE:Syncope:0.64), (RABEPRAZOLE SODIUM:Syncope:0.09), (SACUBITRILVALSARTAN:Syncope:0.64), (SOTALOL:Syncope:0.07), (TELMISARTAN:Syncope:0.16), (TERIFLUNOMIDE:Syncope:0.59), (THALIDOMIDE:Sy neope:0.04), (VARENICLINE TARTRATE:Syncope:0.75), (PYRAZINAMIDE:Synovitis:0.59), (NEVIRAPINE:Syphilis:0.04), (ACETAMINOPHENPROPOXYPHENE HYDROCHLORIDE:Systemic lupus erythematosus:0.1), (ACETAMINOPHENPROPOXYPHENE NAPSYLATE:Systemic lupus erythematosus:0.36), (ACTIVATED CHARCOAL:Systemic lupus erythematosus:0.7), (ADO-TRASTUZUMAB EMTANSINE:Systemic lupus erythematosus:0.05), (AGALSIDASE BETA:Systemic lupus erythematosus:0.99), (ALEMTUZUMAB:Systemic lupus erythematosus:1.32), (ALOGLIPTIN BENZOATE:Systemic lupus erythematosus:0.08), (AMANTADINE HYDROCHLORIDE:Systemic lupus erythematosus:0.25), (AMILORIDE HYDROCHLORIDEHYDROCHLOROTHIAZIDE:Systemic lupus erythematosus:0.41), (AMINO ACIDSDEXTROSEELECTROLYTES NOS:Systemic lupus erythematosus:0.02), (AMISULPRIDE:Systemic lupus erythematosus:0.41), (AMLODIPINE BESYLATEATORVASTATIN CALCIUM:Systemic lupus erythematosus:0.46), (AMLODIPINE BESYLATEIRBESARTAN:Systemic lupus erythematosus:0.31), (AMLODIPINEVALSARTAN:Systemic lupus erythematosus:0.66), (AMMONIUM LACTATE:Systemic lupus erythematosus:0.25), (AMOXAPINE:Systemic lupus erythematosus:0.31), (AMPICILLIN SODIUM:Systemic lupus erythematosus:0.03), (APREPITANT:Systemic lupus erythematosus:0.2), (BALSALAZIDE DISODIUM:Systemic lupus erythematosus:0.09), (BETAMETHASONE DIPROPIONATECALCIPOTRIENECALCIPOTRIENE HYDRATE:Systemic lupus erythematosus:0.66), (BETAMETHASONE SODIUM PHOSPHATE:Systemic lupus erythematosus:0.1), (BETAMETHASONECALCIPOTRIENE:Systemic lupus erythematosus:0.66), (BICALUTAMIDE:Systemic lupus erythematosus:0.04), (BRINZOLAMIDE:Systemic lupus erythematosus:0.91), (CALCIUM CARBONATEERGOCALCIFEROLRETINOL:Systemic lupus erythematosus:0.14), (CALCIUM\MAGNESIUM\ZINC:Systemic lupus erythematosus:0.49), (CANAGLIFLOZINMETFORMIN HYDROCHLORIDE:Systemic lupus erythematosus:0.59), (CAPSAICIN:Systemic lupus erythematosus:0.05), (CARBOHYDRATESPOTASSIUM CHLORIDE\SODIUM CHLORIDE\SODIUM LACTATE:Systemic lupus erythematosus:0.26), (CARFILZOMIB:Systemic lupus erythematosus:1.29), (CARVEDILOL PHOSPHATE:Systemic lupus erythematosus:1.88), (CASANTHRANOLDOCUSATE SODIUM:Systemic lupus erythematosus:0.2), (CEFPODOXIME PROXETIL:Systemic lupus erythematosus:0.19), (CEFTERAM PIVOXIL:Systemic lupus erythematosus:0.01), (CEPHARANTHINE:Systemic lupus erythematosus:0.35), (CETUXIMAB:Systemic lupus erythematosus:0.17), (CHLOROTHIAZIDE:Systemic lupus erythematosus:0.11), (CHLORPHENIRAMINEHYDROCODONE:Systemic lupus erythematosus:0.3), (CLOZAPINE:Systemic lupus erythematosus:1.22), (CODEINE PHOSPHATEGUAIFENESIN:Systemic lupus erythematosus:0.4), (CYPROHEPTADINE:Systemic lupus erythematosus:0.71), (DACLIZUMAB:Systemic lupus erythematosus:1.23), (DALTEPARIN:Systemic lupus erythematosus:0.5), (DAPAGLIFLOZIN:Systemic lupus erythematosus:0.4), (DAPAGLIFLOZIN PROPANEDIOL:Systemic lupus erythematosus:0.61), (DEXTROAMPHETAMINE SULFATE:Systemic lupus erythematosus:0.56), (DILAZEP:Systemic lupus erythematosus:1.19), (DIPHENIDOL HYDROCHLORIDE:Systemic lupus erythematosus:0.54), (DIPYRONEPROMETHAZINE HYDROCHLORIDE:Systemic lupus erythematosus:0.12), (DUTASTERIDE:Systemic lupus erythematosus:1.07), (ECONAZOLE:Systemic lupus erythematosus:0.77), (EMPAGLIFLOZINLINAGLIPTIN:Systemic lupus erythematosus:0.39), (ERTAPENEM SODIUM:Systemic lupus erythematosus:0.23), (ETHINYL ESTRADIOLNORETHINDRONE:Systemic lupus erythematosus:0.34), (ETRETINATE:Systemic lupus erythematosus:1.22), (EVOLOCUMAB:Systemic lupus erythematosus:2.08), (FAROPENEM SODIUM:Systemic lupus erythematosus:0.23), (FENOTEROL HYDROBROMIDE:Systemic lupus erythematosus:0.12), (FLURAZEPAMFLURAZEPAM HYDROCHLORIDE:Systemic lupus erythematosus:0.22), (FLUTICASONE PROPIONATEFORMOTEROL FUMARATE:Systemic lupus erythematosus:0.22), (FLUVOXAMINE MALEATE:Systemic lupus erythematosus:0.59), (FORMOTEROL:Systemic lupus erythematosus:0.76), (FORMOTEROL FUMARATE:Systemic lupus erythematosus:1.06), (FORMOTEROL FUMARATE\MOMETASONE FUROATE:Systemic lupus erythematosus:0.63), (FOSFOMYCIN:Systemic lupus erythematosus:0.07), (FOSPHENYTOINFOSPHENYTOIN SODIUM:Systemic lupus erythematosus:0.17), (FUSIDIC ACID:Systemic lupus erythematosus:0.84), (GENTAMICIN:Systemic lupus erythematosus:1.16), (GIMERACILOTERACILTEGAFUR:Systemic lupus erythematosus:0.04), (GLYCERIN:Systemic lupus erythematosus:0.11), (GLYCOPYRROLATE:Systemic lupus erythematosus:0.58), (GLYCOPYRROLATEINDACATEROL MALEATE:Systemic lupus erythematosus:0.17), (GLYCOPYRRONIUM:Systemic lupus erythematosus:0.04), (GLYCOPYRRONIUMINDACATEROL:Systemic lupus erythematosus:0.87), (GUANFACINE:Systemic lupus erythematosus:0.25), (GUANFACINE HYDROCHLORIDE:Systemic lupus erythematosus:0.11), (HYDROCHLOROTHIAZIDETELMISARTAN:Systemic lupus erythematosus:0.87), (HYDROCORTISONE ACETATE:Systemic lupus erythematosus:1.11), (IGURATIMOD:Systemic lupus erythematosus:0.3), (INDACATEROL:Systemic lupus erythematosus:0.31), (INFLIXIMAB-DYYB:Systemic lupus erythematosus:0.79), (INSULIN BEEF:Systemic lupus erythematosus:0.31), (INSULIN GLULISINE:Systemic lupus erythematosus:0.3), (INTERFERON GAMMA-1B:Systemic lupus erythematosus:0.09), (IODINE:Systemic lupus erythematosus:0.32), (LABETALOL HYDROCHLORIDE:Systemic lupus erythematosus:0.88), (LACTASE:Systemic lupus erythematosus:0.63), (LERCANIDIPINE:Systemic lupus erythematosus:1.66), (LERCANIDIPINE HYDROCHLORIDE:Systemic lupus erythematosus:0.82), (LEVALBUTEROL:Systemic lupus erythematosus:0.32), (LEVOCARNITINE HYDROCHLORIDE:Systemic lupus erythematosus:0.26), (LEVOLEUCOVORIN CALCIUM:Systemic lupus erythematosus:0.14), (LIDOCAINEPRILOCAINE:Systemic lupus erythematosus:0.91), (LOMITAPIDE MESYLATE:Systemic lupus erythematosus:0.56), (LULICONAZOLE:Systemic lupus erythematosus:0.09), (LYSOZYME HYDROCHLORIDE:Systemic lupus erythematosus:0.4), (MAXACALCITOL:Systemic lupus erythematosus:0.48), (METFORMIN HYDROCHLORIDEVILDAGLIPTIN:Systemic lupus erythematosus:0.21), (METHENOLONE ENANTHATE:Systemic lupus erythematosus:0.52), (METILDIGOXIN:Systemic lupus erythematosus:0.43), (MEXILETINE HYDROCHLORIDE:Systemic lupus erythematosus:0.3), (MOEXIPRIL HYDROCHLORIDE:Systemic lupus erythematosus:0), (NADROPARIN CALCIUM:Systemic lupus erythematosus:0.38), (NALOXONE HYDROCHLORIDEOXYCODONE HYDROCHLORIDE:Systemic lupus erythematosus:1.15), (NALOXONE HYDROCHLORIDETILIDINE HYDROCHLORIDE:Systemic lupus erythematosus:0.61), (NINTEDANIB:Systemic lupus erythematosus:0.41), (NOREPINEPHRINE BITARTRATE:Systemic lupus erythematosus:0.47), (OLMESARTAN:Systemic lupus erythematosus:0.77), (OLODATEROL HYDROCHLORIDETIOTROPIUM BROMIDE MONOHYDRATE:Systemic lupus erythematosus:0.9), (PARATHYROID HORMONE:Systemic lupus erythematosus:0.3), (PARICALCITOL:Systemic lupus erythematosus:0.43), (PATIROMER:Systemic lupus erythematosus:0.18), (PEMETREXED DISODIUM:Systemic lupus erythematosus:0.4), (PENICILLIN V:Systemic lupus erythematosus:0.37), (PENICILLIN V POTASSIUM:Systemic lupus erythematosus:0.09), (PHENYTOIN SODIUM:Systemic lupus erythematosus:1.39), (PILSICAINIDE HYDROCHLORIDE:Systemic lupus erythematosus:0.88), (PIMOBENDAN:Systemic lupus erythematosus:0.72), (PLANTAGO SEED:Systemic lupus erythematosus:0.84), (PONATINIB:Systemic lupus erythematosus:1.3), (PROPAFENONE HYDROCHLORIDE:Systemic lupus erythematosus:0.55), (PROTRIPTYLINE HYDROCHLORIDE:Systemic lupus erythematosus:0.27), (PSYLLIUM HUSK:Systemic lupus erythematosus:0.58), (RIVASTIGMINE:Systemic lupus erythematosus:0.44), (ROFLUMILAST:Systemic lupus erythematosus:1.26), (SACCHAROMYCES CEREVISIAE:Systemic lupus erythematosus:0.18), (SALMETEROL:Systemic lupus erythematosus:0.2), (SALMETEROL XINAFOATE:Systemic lupus erythematosus:0.51), (SODIUM PHOSPHATE, DIBASIC\SODIUM PHOSPHATE, MONOBASIC:Systemic lupus erythematosus:0.25), (SODIUM POLYSTYRENE SULFONATE:Systemic lupus erythematosus:0.06), (SULFAMETHOXAZOLE:Systemic lupus erythematosus:1.18), (SUPLATAST TOSILATE:Systemic lupus erythematosus:0.26), (TERAZOSIN HYDROCHLORIDE:Systemic lupus erythematosus:0.21), (TERBUTALINE:Systemic lupus erythematosus:0.37), (TERBUTALINE SULFATE:Systemic lupus erythematosus:1.32), (THEOPHYLLINE:Systemic lupus erythematosus:0.02), (TOFACITINIB:Systemic lupus erythematosus:0.32), (TOLTERODINE:Systemic lupus erythematosus:0.16), (TOSUFLOXACIN TOSYLATE:Systemic lupus erythematosus:0.01), (UMECLIDINIUM BROMIDE:Systemic lupus erythematosus:0.41), (VITAMIN A:Systemic lupus erythematosus:1), (ZILEUTON:Systemic lupus erythematosus:0.33), (DILTIAZEM:Systemic scleroderma:0.05), (CALCIUM:Systemic sclerosis:0.2), (FLUTICASONE PROPIONATE:Systemic sclerosis:0.16), (SELEXIPAG:Systemic sclerosis:0.77), (IBRITUMOMAB TIUXETAN:T-cell lymphoma:0.4), (THALIDOMIDE:T-cell lymphoma:0.24), (IBRITUMOMAB TIUXETAN:T-cell type acute leukaemia:0.39), (.ALPHA.1-PROTEINASE INHIBITOR HUMAN:Tachycardia:0.58), (ACETAMINOPHEN\CAFFEINEOPIUM:Tachycardia:0.05), (AC ETAMINOPHENOXYCODONEOXYCODONE HYDROCHLORIDE:Tachycardia:0.67), (ACETAMINOPHENTRAMADOL:Tachycardia:0.71), (ACTIVATED CHARCOAL:Tachycardia:0.14), (ALBIGLUTIDE:Tachycardia:0.17), (ALCOHOL:Tachycardia: 0.73), (ALENDRONIC ACID:Tachycardia:1.14), (ALISKIREN HEMIFUMARATE:Tachycardia:0.14), (AMLODIPINE BESYLATEATORVASTATIN CALCIUM:Tachycardia:0.1), (AMLODIPINE BESYLATEOLMESARTAN MEDOXOMIL:Tachycardia:0.14), (AMLODIPINEPERINDOPRIL:Tachycardia:0.6), (AMOXI CILLINCLAVULANIC ACID:Tachycardia:0.64), (ASPIRINMAGNESIUM OXIDE:Tachycardia:0.14), (AZELASTINE HYDROCHLORIDE:Tachycardia:0.2), (BENAZEPRILBENAZEPRIL HYDROCHLORIDE:Tachycardia:0.92), (BETAMETHASONECALCIPOTRIENE:Tachycardia: 0.01), (BICALUTAMIDE:Tachycardia:0.36), (BIFIDOBACTERIUM LONGUM SUBSP. INFANTIS:Tachycardia:0.14), (BILASTINE:Tachycardia:0.18), (BLINATUMOMAB:Tachycard ia:0.17), (BUPIVACAINE HYDROCHLORIDE:Tachycardia:0.07), (BUPRENORPHINE HYDROCHLORIDENALOXONE HYDROCHLORIDE:Tachycardia:0.79), (BUTYLSCOPOLAMINE BROMIDE:Tachycardia:0.88), (CALCITONIN:Tachycardia:0.15), (CALCITONIN SALMON:Tachycardia:0.29), (CALCIUM ACETATE:Tachycardia:1.31), (CALCIUM CARBONATEVITAMIN D:Tachycardia:0.86), (CALCIUM\MAGNESIUM\ZINC:Tachycardia:0.1), (CANAGLIFLOZIN METFORMIN HYDROCHLORIDE:Tachycardia:0.41), (CEFOTAXIMECEFOTAXIME SODIUM:Tachycardia:0.05), (CEFUROXIME AXETIL:Tachycardia:0.22), (CETIRIZINE HYDROCHLORIDEPSEUDOEPHEDRINE HYDROCHLORIDE:Tachycardia:0.18), (CHONDROITIN SULFATE (BOVINE)GLUCOSAMINE HYDROCHLORIDE:Tachycardia:0.43), (CICLESONIDE:Tachycardia:0.58), (CLINDAMYCIN: Tachycardia:1.21), (COD LIVER OIL:Tachycardia:0.11), (CORTICOTROPIN:Tachycardia:0.41), (CYAMEMAZINE:Tachycardia: 0.4), (CYPROHEPTADINE:Tachycardia:0.27), (DANAZOL:Tachycardia:0.6), (DAPAGLIFLOZ IN PROPANEDIOL:Tachycardia:0.37), (DEXAMETHASONE ACETATE:Tachycardia:0.7), (DEXAMETHASONE SODIUM PHOSPHATE:Tachycardia:0.48), (DONEPEZIL:Tachycardia:0.45), (DOXORUBICIN HYDROCHLORIDE:Tachycardia:0.05), (DOXYLAMINE SUCCINATE:Tachycardia:0.18), (DULAGLUTIDE:Tachycardia:0.03), (ECONAZOLE:Tachyca rdia:1.1), (ELDECALCITOL:Tachycardia:0.22), (ELOTUZUMAB:Tachycardia:0.47), (ELTROM BOPAG OLAMINE:Tachycardia:0.05), (EMPAGLIFLOZINLINAGLIPTIN:Tachycardia:0.38), (FELOD IPINE:Tachycardia:0.28), (FENOTEROL HYDROBROMIDE:Tachycardia:0.3), (FERROUS GLUCONATE:Tachycardia:0.08), (FERROUS SULFATEFOLIC ACID:Tachycardia:0.07), (FERROUS SULFATEGLYCINE:Tachycardia:0.52), (FLUOCINONIDE:Tachycardia:0.13), (FORMOTER OL:Tachycardia:0.84), (FORMOTEROL FUMARATE\MOMETASONE FUROATE:Tachycardia:0.16), (FOSFOMYCIN:Tachycardia:0.03), (FOSPHENYTOINFOSPHE NYTOIN SODIUM:Tachycardia:0.55), (FUSIDIC ACID:Tachycardia:0.63), (GIMERACILOTERACILTEGAFUR:Tachycardia:0.04), (GLYCOP YRROLATEINDACATEROL MALEATE:Tachycardia:0.03), (GLYCOPYRRONIUM:Tachycardia:0), (HERBALSTURMERI C:Tachycardia:0.51), (IDARUCIZUMAB:Tachycardia:0.03), (ILOPROST:Tachycardia:1.18), (IM ATINIB MESYLATE:Tachycardia:1.97), (IMIPRAMINE HYDROCHLORIDE:Tachycardia:0.75), (IMMUNE GLOBULIN NOS:Tachycardia:0.2), (INFLIXIMAB-DYYB:Tachycardia:0.52), (INSULIN GLULISINE:Tachycardia:0.27), (INTERFERON:Tachycardia:0.12), (INTERFERON ALFA-2A:Tachycardia:0.24), (IOPAMIDOL:Tachycardia:0.04), (ISOSORBIDE:Tachycardia:1.32), (IX AZOMIB:Tachycardia:0.46), (KETAMINE:Tachycardia:0.07), (KETAMINE HYDROCHLORIDE:Tachycardia:0.35), (KETOROLACKETOROLAC TROMETHAMINE:Tachycardia:0.36), (LACTASE:Tachycardia:0.09), (LEVOLEUCOVORIN CALCIUM:Tachycardia:0.18), (LURASIDONE HYDROCHLORIDE:Tachycardia:0.24), (MECLIZINEMECLIZINE HYDROCHLORIDE:Tachycardia:0.09), (MEGESTROL:Tachycardia:0), (MEPERIDINE:Tachyc ardia:0.7), (METHENAMINE HIPPURATE:Tachycardia:0.99), (METHENOLONE ENANTHATE:Tachycardia:0.07), (METHYLPREDNISOLONE ACETATE:Tachycardia:1.16), (MIDODRINE HYDROCHLORIDE:Tachycardia:0.34), (MINOCYCLINEMINOCYCLINE HYDROCHLORIDE:Tachycardia:1), (MUPIROCINMUPIROCIN CALCIUM:Tachycardia:0.43), (NALOXONE:Tachycardia:0.33), (NALTREXONE:Tachycardia: 0.12), (NATEGLINIDE:Tachycardia:0.35), (NITROFURANTOINNITROFURANTOIN MONOHYDRATE:Tachycardia:0.26), (NOREPINEPHRINE BITARTRATE:Tachycardia:0.5), (OLMESARTAN:Tachycardia:0.08), (OMEGA-3-ACID ETHYL ESTERS:Tachycardia:1.62), (OSELTAMIVIR:Tachycardia:0.62), (PARICALCITOL:Tachycardi a:0.38), (PATIROMER:Tachycardia:0.27), (PEGINTERFERON BETA-1A:Tachycardia:0.98), (PEMETREXED DISODIUM:Tachycardia:0.54), (PENICILLIN:Tachycardia:1.09), (PIMOBENDAN:Tachycardia: 0.31), (POLYETHYLENE GLYCOL 4000:Tachycardia:0.36), (POSACONAZOLE:Tachycardia:0.43), (PREDNISOLONE ACETATE:Tachycardia:0.17), (PREDNISOLONE SODIUM PHOSPHATE:Tachycardia:0.02), (PRISTINAMYCIN:Tachycardia:0.22), (PYRAZINAMIDE:Ta chycardia:0.75), (QUINAPRIL:Tachycardia:0.15), (QUINAPRILQUINAPRIL HYDROCHLORIDE:Tachycardia:0), (RADIATION THERAPY:Tachycardia:0.06), (RANOLAZINE:Tachycardia:1.84), (REGORAFENIB:Tachycard ia:0.46), (RETINOL:Tachycardia:0.07), (RIOCIGUAT:Tachycardia:0.29), (RIVASTIGMINE:Tac hycardia:0.67), (RIZATRIPTAN:Tachycardia:0.11), (RIZATRIPTAN BENZOATE:Tachycardia:1.19), (SELEXIPAG:Tachycardia:0.07), (SEVELAMER CARBONATE:Tachycardia:0.32), (SODIUM CITRATE:Tachy cardia:0.07), (SODIUM FLUORIDE:Tachycardia:0.22), (SODIUM PHOSPHATE, DIBASIC\SODIUM PHOSPHATE, MONOBASIC:Tachycardia:0.22), (SUFENTANIL CITRATE:Tachycardia:0.14), (TEMOZOLOMIDE:Tachycardia:0.1), (TERBINAFINE HYDROCHLORIDE:Tachycardia:0.51), (TESTOSTERONE CYPIONATE:Tachycardia:0.05), (TESTOSTERONE UNDECANOATE:Tachycardia:0.28), (TETRACYCLINE:Tachycardia:0.51), (THYROID, PORCINETHYROID, UNSPECIFIED:Tachycardia:0), (TICLOPIDINE:Tachycardia:0.1), (TIGECYCLINE:Tachycardi a:0.12), (TINZAPARIN:Tachycardia:0.33), (TRIHEXYPHENIDYL:Tachycardia:0.07), (UMECLI DINIUM BROMIDEVILANTEROL TRIFENATATE:Tachycardia:0.83), (VILDAGLIPTIN:Tachycardia:0.18), (VINORELBINE TARTRATE:Tachycardia:0.22), (ALCOHOL:Tardive dyskinesia:0.4), (ALLOPURINOL:Tardive dyskinesia:0.24), (AMOXICILLIN:Tardive dyskinesia:0.06), (ANASTROZOLE:Tardive dyskinesia:0.15), (ARIPIPRAZOLE LAUROXIL:Tardive dyskinesia:0.06), (BISACODYL OR DOCUSATE SODIUM:Tardive dyskinesia:0.02), (BUPRENORPHINE HYDROCHLORIDE:Tardive dyskinesia:0.21), (BUPRENORPHINE HYDROCHLORIDENALOXONE HYDROCHLORIDE:Tardive dyskinesia:0.15), (CALCIUM CARBONATECHOLECALCIFEROL:Tardive dyskinesia:0.05), (CHOLESTYRAMINE:Tardive dyskinesia:0.08), (DALFAMPRIDINE:Tardive dyskinesia:0.16), (ENALAPRIL MALEATE:Tardive dyskinesia:0.15), (EVOLOCUMAB:Tardive dyskinesia:0.1), (FERROUS SULFATEFERROUS SULFATE, DRIED:Tardive dyskinesia:0.53), (FLUOXETINEFLUOXETINE HYDROCHLORIDE:Tardive dyskinesia:0.42), (GUANFACINE:Tardive dyskinesia:0.39), (HYDROMORPHONE HYDROCHLORIDE:Tardive dyskinesia:0.75), (IBRUTINIB:Tardive dyskinesia:0.6), (LABETALOLLABETALOL HYDROCHLORIDE:Tardive dyskinesia:0.31), (LACOSAMIDE:Tardive dyskinesia:0.05), (METHYLPREDNISOLONE SODIUM SUCCINATE:Tardive dyskinesia:0.01), (METRONIDAZOLE:Tardive dyskinesia:0.22), (MOXIFLOXACIN HYDROCHLORIDE:Tardive dyskinesia:0.19), (NAPROXEN SODIUM:Tardive dyskinesia:0.15), (NEBIVOLOL HYDROCHLORIDE:Tardive dyskinesia:0.04), (NORTRIPTYLINE:Tardive dyskinesia:0.45), (OXYGEN:Tardive dyskinesia:0.42), (PHENYTOIN:Tardive dyskinesia:0.23), (POLYETHYLENE GLYCOLS:Tardive dyskinesia:0.27), (PROMETHAZINE HYDROCHLORIDE:Tardive dyskinesia:0.12), (RANOLAZINE:Tardive dyskinesia:0.92), (SCOPOLAMINE:Tardive dyskinesia:0.17), (SUMATRIPTANSUMATRIPTAN SUCCINATE:Tardive dyskinesia:0.24), (TELMISARTAN:Tardive dyskinesia:0.12), (TERIFLUNOMIDE:Tardive dyskinesia:0.38), (VARENICLINE TARTRATE:Tardive dyskinesia:1.48), (ZOPICLONE:Tardive dyskinesia:0.1), (AMIODARONE:Temporal arteritis:0.63), (BUMETANIDE:Temporal arteritis:0.18), (CIPROFLOXACIN:Temporal arteritis:0.04), (COLCHICINE:Temporal arteritis:0.15), (CYCLOBENZAPRINE:Temporal arteritis:0.02), (DASATINIB:Temporal arteritis:0.01), (EVEROLIMUS:Temporal arteritis:0.14), (HYDROXYCHLOROQUINE SULFATE:Temporal arteritis:0.57), (IRON:Temporal arteritis:0.24), (LERCANIDIPINE:Temporal arteritis:0.43), (LOVASTATIN:Temporal arteritis:1.01), (MAGNESIUM:Temporal arteritis:0.29), (NEBIVOLOL:Temporal arteritis:0.77), (RALOXIFENE HYDROCHLORIDE:Temporal arteritis:0.08), (SACUBITRILVALSARTAN:Temporal arteritis:0.27), (SECUKINUMAB:Temporal arteritis:0.38), (TIMOLOL:Temporal arteritis:0.13), (TOFACITINIB CITRATE:Temporal arteritis:0.3), (TRIAMCINOLONE ACETONIDE:Temporal arteritis:0.37), (ZOLPIDEMZOLPIDEM TARTRATE:Temporal arteritis:0.13), (CALCIUM CARBONATECHOLECALCIFEROL:Temporomandibular joint syndrome:0.02), (DILTIAZEM:Temporomandibular joint syndrome:0.32), (DIVALPROEX SODIUM:Temporomandibular joint syndrome:0.17), (EVOLOCUMAB:Temporomandibular joint syndrome:0.44), (LANSOPRAZOLE:Temporomandibular joint syndrome:0.27), (METHYLPREDNISOLONE SODIUM SUCCINATE:Temporomandibular joint syndrome:0.23), (OLANZAPINE:Temporomandibular joint syndrome:0.02), (PANTOPRAZOLE:Temporomandibular joint syndrome:0.25), (RANITIDINE HYDROCHLORIDE:Temporomandibular joint syndrome:0.15), (SELEXIPAG:Temporomandibular joint syndrome:0.16), (TERIFLUNOMIDE:Temporomandibular joint syndrome:0.36), (TOCILIZUMAB:Temporomandibular joint syndrome:0.06), (BISOPROLOL FUMARATE:Tendonitis:0.1), (CALCIUM CARBONATECHOLECALCIFEROL:Tendonitis:0.43), (CLINDAMYCINCLINDAMYCIN PHOSPHATE:Tendonitis:0.02), (DEXLANSOPRAZOLE:Tendonitis:0.54), (HYDROXYCHLO ROQUINE DIPHOSPHATE:Tendonitis:0.16), (LOVASTATIN:Tendonitis:0.1), (METHYLPREDNISOLON E SODIUM SUCCINATE:Tendonitis:0.33), (RAMIPRIL:Tendonitis:0.49), (TAMSULOSIN:Tendonitis:0.27), (TERIFLUNOMIDE:Tendonitis:0.21), (TOCILIZUMAB:Tendonitis:0.48), (WARFARIN:Tendo nitis:0.46), (DOCUSATE SODIUM:Tension:0.06), (LOVASTATIN:Tension:0.14), (PAROXETINE:Tension:0.1), (SACUB ITRILVALSARTAN:Tension:0.04), (TERIFLUNOMIDE:Tension:0.3), (ARMODAFINIL:Tensi on headache:0.36), (CALCIUM CARBONATECHOLECALCIFEROL:Tension headache:0.04), (CERTOLIZUMAB PEGOL:Tension headache:0.6), (CIPROFLOXACIN HYDROCHLORIDE:Tension headache:0.08), (DALFAMPRIDINE:Tension headache:0.77), (ESZOPICLONE:Tension headache:0.33), (EVOLOCUMAB:Tension headache:0.36), (FERROUS SULFATE:Tension headache:0.21), (FERROUS SULFATEFERROUS SULFATE, DRIED:Tension headache:0.15), (HYDROCHLOROTHIAZIDELISINOPRIL:Tension headache:0.03), (IBRUTINIB:Tension headache:0.16), (MELATONIN:Tension headache:0.49), (PAROXETINE:Tension headache:0.13), (PRAMIPEXOLE DIHYDROCHLORIDE:Tension headache:0.19), (RIZATRIPTAN BENZOATE:Tension headache:0.08), (TAMSULOSIN:Tension headache:0.1), (AMIODARONE:Therapeutic procedure:0.55), (AMIODARONE HYDROCHLORIDE:Therapeutic procedure:0.5), (ANASTROZOLE:Therapeutic procedure:0.6), (BICALUTAMIDE:Therapeutic procedure:0.46), (BUMETANIDE:Therapeutic procedure:0.39), (CALCIUM:Therapeutic procedure:0.41), (CALCIUM CARBONATECHOLECALCIFEROL:Therapeutic procedure:0.2), (CLINDAMYCINCLINDAMYCIN PHOSPHATE:Therapeutic procedure:0.41), (DASATINIB:Therapeutic procedure:0.25), (DEXLANSOPRAZOLE:Therapeutic procedure:0.37), (DILTIAZEM:Therapeutic procedure:0.15), (DILTIAZEM HYDROCHLORIDE:Therapeutic procedure:0.27), (EVEROLIMUS:Therapeutic procedure:0.54), (EVOLOCUMAB:Therapeutic procedure:0.26), (EXEMESTANE:Therapeutic procedure:0.03), (GABAPENTIN:Therapeutic procedure:0), (IBRUTINIB:Therapeutic procedure:0.44), (LERCANIDIPINE:Therapeutic procedure:0.12), (MONTELUKAST SODIUM:Therapeutic procedure:0.35), (OLANZAPINE:Therapeutic procedure:0.1), (OXYGEN:Therapeutic procedure:0.07), (POTASSIUM:Therapeutic procedure:0.13), (SACUBITRILVALSARTAN:Therapeutic procedure:0.2), (SERTRALINE HYDROCHLORIDE:Therapeutic procedure:0.9), (TOCILIZUMAB:Therapeutic procedure:0.16), (TRIAMCINOLONE ACETONIDE:Therapeutic procedure:0.46), (PANTOPRAZOLE:Therapeutic skin care topical:0.06), (TERIFLUNOMIDE:Therapeutic skin care topical:0.05), (NAPROXEN:Therapy naive:0.1), (UNSPECIFIED INGREDIENT:Therapy naive:0.53), (VERAPAMIL HYDROCHLORIDE:Therapy naive:0.09), (ACETAMINOPHENOXYCODONEOXYCODONE HYDROCHLORIDE:Thrombocytopenia:0.03), (ACETAZOLAMIDE:Thrombocytopenia:0.03), (ACLIDINIUM BROMIDE:Thrombocytopenia:0.35), (ALCOHOL:Thrombocytopenia:0.18), (ALENDRONIC ACID:Thrombocytopenia:0.32), (AMANTADINE:Thrombocytopenia:0.31), (AMILORIDE HYDROCHLORIDEHYDROCHLOROTHIAZIDE:Thrombocytopenia:0.18), (AMISULPRIDE: Thrombocytopenia:0.05), (ARMODAFINIL:Thrombocytopenia:0.12), (BELATACEPT:Thromb ocytopenia:0.07), (BENAZEPRIL HYDROCHLORIDE:Thrombocytopenia:0.61), (BUDESONIDEFORMOTEROL FUMARATE:Thrombocytopenia:0.24), (BUPROPION:Thrombocytopenia:0.65), (BUTYLSCOP OLAMINE BROMIDE:Thrombocytopenia:0.15), (CALCITONIN:Thrombocytopenia:0.22), (CALCITONIN SALMON:Thrombocytopenia:0), (CALCIUM CITRATE:Thrombocytopenia:0.19), (CANAKINUMAB:Thrombocytopenia:0.52), (CANDESAR TAN CILEXETILHYDROCHLOROTHIAZIDE:Thrombocytopenia:1.13), (CARVEDILOL PHOSPHATE:Thrombocytopenia:0.53), (CERITINIB:Thrombocytopenia:0.04), (CERTOLIZUMAB PEGOL:Thrombocytopenia:1.35), (CETUXIMAB:Thrombocytopenia:0.97), (CRIZOTINIB:Thro mbocytopenia:0.77), (CYCLOBENZAPRINE:Thrombocytopenia:0.47), (DABIGATRAN:Throm bocytopenia:0.11), (DABRAFENIB:Thrombocytopenia:0.22), (DACLIZUMAB:Thrombocytopen ia:0.56), (DALFAMPRIDINE:Thrombocytopenia:0.19), (DEXLANSOPRAZOLE:Thrombocytop enia:0.97), (DICLOFENAC:Thrombocytopenia:1.32), (DIGITOXIN:Thrombocytopenia:0.44), (DI PYRONE:Thrombocytopenia:0.07), (DOCUSATE CALCIUM:Thrombocytopenia:0.3), (DOFETILIDE:Thrombocytopenia:0.15), (EDOXABAN TOSYLATE:Thrombocytopenia:0.65), (ELETRIPTAN HYDROBROMIDE:Thrombocytopenia:0.16), (ELOTUZUMAB:Thrombocytopenia:0.61), (EPIR UBICIN:Thrombocytopenia:0.96), (ERYTHROMYCIN:Thrombocytopenia:0.52), (ESTROGENS, CONJUGATED:Thrombocytopenia:0.05), (ETHAMBUTOL HYDROCHLORIDE:Thrombocytopenia:0.95), (EVOLOCUMAB:Thrombocytopenia:0.82), (FE SOTERODINE FUMARATE:Thrombocytopenia:0.52), (FLECAINIDE:Thrombocytopenia:1.06), (FLUINDION E:Thrombocytopenia:0.23), (FLUOXETINEFLUOXETINE HYDROCHLORIDE:Thrombocytopenia:0.44), (FLUTICASONE FUROATESALMETEROL XINAFOATE:Thrombocytopenia:0.71), (FLUTICASONEFLUTICASONE PROPIONATE:Thrombocytopenia:0.22), (FORMOTEROL:Thrombocytopenia:0.2), (FORMOTE ROL FUMARATE:Thrombocytopenia:0.45), (FULVESTRANT:Thrombocytopenia:0.54), (FUSIDIC ACID:Thrombocytopenia:0.28), (GLYCOPYRRONIUMINDACATEROL:Thrombocytopenia:00.52), (HEPARIN CALCIUM:Thrombocytopenia:0.34), (HYDROCHLOROTHIAZIDETELMISARTAN:Thromb ocytopenia:0.2), (HYDROCHLOROTHIAZIDEVALSARTAN:Thrombocytopenia:0.39), (INDA PAMIDE:Thrombocytopenia:0.31), (INDAPAMIDEPERINDOPRIL:Thrombocytopenia:0.72), (I NDOMETHACIN:Thrombocytopenia:0.15), (INTERFERON ALFA-2A:Thrombocytopenia:0.49), (IRINOTECAN HYDROCHLORIDE:Thrombocytopenia:0.65), (LAPINE T-LYMPHOCYTE IMMUNE GLOBULIN:Thrombocytopenia:0.29), (MESNA:Thrombocytopenia:0.46), (METAXALONE:Thr ombocytopenia:0.16), (METHENAMINE HIPPURATE:Thrombocytopenia:0.23), (METHENOLONE ENANTHATE:Thrombocytopenia:0.05), (MICAFUNGIN:Thrombocytopenia:1.06), (MINOCYC LINEMINOCYCLINE HYDROCHLORIDE:Thrombocytopenia:0.36), (NITROFURANTOIN:Thrombocytopenia:1.09), (OCTREOTIDE ACETATE:Thrombocytopenia:0.23), (OLAPARIB:Thrombocytopenia:0.27), (OXYBUTYNIN CHLORIDE:Thrombocytopenia:0.22), (PAMIDRONATE DISODIUM:Thrombocytopenia:0.11), (PANTOPRAZOLE MAGNESIUM:Thrombocytopenia:0.92), (PAROXETINE:Thrombocytopenia:0.6), (PEGINTER FERON BETA-1A:Thrombocytopenia:0.05), (PEMETREXED:Thrombocytopenia:0.64), (PENICILLIN:Thromb ocytopenia:0.58), (PITAVASTATIN CALCIUM:Thrombocytopenia:0.35), (PLANTAGO SEED:Thrombocytopenia:0.04), (PRAMIPEXOLE DIHYDROCHLORIDE:Thrombocytopenia:0.34), (PREDNISOLONE SODIUM PHOSPHATE:Thrombocytopenia:0.31), (PRIMIDONE:Thrombocytopenia:0.35), (PROBIOTICS NOS:Thrombocytopenia:0.94), (REGORAFENIB:Thrombocytopenia:0.89), (ROFLUMILAST:T hrombocytopenia:0.48), (ROPINIROLE HYDROCHLORIDE:Thrombocytopenia:0.05), (SACUBITRILVALSARTAN:Thrombocytopen ia:0.87), (SECUKINUMAB:Thrombocytopenia:1.01), (SIPULEUCEL-T:Thrombocytopenia:0.03), (SUMATRIPTANSUMATRIPTAN SUCCINATE:Thrombocytopenia:0), (TERBUTALINE:Thrombocytopenia:0.07), (TERBUTALINE SULFATE:Thrombocytopenia:0.45), (TETRACYCLINE:Thrombocytopenia:0.45), (THALIDOM IDE:Thrombocytopenia:1.68), (TINZAPARIN:Thrombocytopenia:0.05), (TIOTROPIUM:Throm bocytopenia:0.6), (TIZANIDINE:Thrombocytopenia:0.32), (TIZANIDINE HYDROCHLORIDE:Thrombocytopenia:0.42), (TOBRAMYCIN:Thrombocytopenia:0.01), (TR AMETINIB:Thrombocytopenia:0.2), (UMECLIDINIUM BROMIDEVILANTEROL TRIFENATATE:Thrombocytopenia:0.41), (VARENICLINE TARTRATE:Thrombocytopenia:0.59), (VEMURAFENIB:Thrombocytopenia:0.37), (VINOREL BINE TARTRATE:Thrombocytopenia:0.19), (EVOLOCUMAB:Thrombocytosis:0), (IBRUTINIB:Thro mbocytosis:0.33), (PRAVASTATIN SODIUM:Thrombocytosis:0), (SACUBITRILVALSARTAN:Thrombocytosis:0.19), (UNSPECI FIED INGREDIENT:Thrombocytosis:0.25), (DILTIAZEM HYDROCHLORIDE:Thrombophlebitis:0.11), (.ALPHA.1-PROTEINASE INHIBITOR HUMAN:Thrombosis:0.65), (ABOBOTULINUMTOXINA:Thrombosis:0.15), (ACEBUTOLOL HYDROCHLORIDE:Thrombosis:0.68), (ACETAMINOPHEN\CAFFEINEOPIUM:Thrombosis: 0.25), (ACETAMINOPHENDIPHENHYDRAMINE HYDROCHLORIDE:Thrombosis:0.29), (ACETAMINOPHENPROPOXYPHENE NAPSYLATE:Thrombosis:0.56), (ACETAZOLAMIDE:Thrombosis:2.19), (ACTIVATED CHARCOAL:Thrombosis:0.65), (ALBUTEROLIPRATROPIUM:Thrombosis:0.59), (ALFUZO SIN:Thrombosis:1.07), (ALISKIREN HEMIFUMARATE:Thrombosis:0.92), (AMANTADINE HYDROCHLORIDE:Thrombosis:0.7), (AMLODIPINE BESYLATETELMISARTAN:Thrombosis:0.08), (AMMONIUM CHLORIDEDEXTROMETHORPHAN\SODIUM CITRATE:Thrombosis:0.2), (AMOXICILLINCLARITHROMYCINESOMEPRAZOLE:Throm bosis:0.38), (ANAKINRA:Thrombosis:0.15), (ANTIHISTAMINES NOS:Thrombosis:0.56), (ARGININE:Thrombosis:0), (ARIPIPRAZOLE LAUROXIL:Thrombosis:0), (ATORVASTATIN CALCIUMEZETIMIBE:Thrombosis:0.09), (AZELNIDIPINE:Thrombosis:0.02), (AZOSEMIDE: Thrombosis:0.43), (BECLOMETHASONE:Thrombosis:0.44), (BENZTROPINE MESYLATE:Thrombosis:0.21), (BETAMETHASONE DIPROPIONATECALCIPOTRIENECALCIPOTRIENE HYDRATE:Thrombosis:0.8), (BETAMETHASONECALCIPOTRIENE:Thrombosis:0.58), (BIL ASTINE:Thrombosis:0.33), (BLINATUMOMAB:Thrombosis:1.11), (BOCEPREVIR:Thrombosis: 0.65), (BUDESONIDEFORMOTEROL:Thrombosis:0.05), (CABAZITAXEL:Thrombosis:0.12), (CALCIUM CARBONATECHOLECALCIFEROLMAGNESIUM CARBONATE:Thrombosis:0.1), (CALCIUM CARBONATEERGOCALCIFEROLRETINOL:Thrombosis:0.59), (CANAGLIFLOZINMETF ORMIN HYDROCHLORIDE:Thrombosis:1.84), (CANAKINUMAB:Thrombosis:1.07), (CAPSAICIN:Th rombosis:0.35), (CEFOTAXIMECEFOTAXIME SODIUM:Thrombosis:0.04), (CELIPROLOL:Thrombosis:0.4), (CERIVASTATIN SODIUM:Thrombosis:0.02), (CHLORPROMAZINE:Thrombosis:0.36), (CHONDROITIN SULFATE (CHICKEN):Thrombosis:0.19), (CLOMIPRAMINE HYDROCHLORIDE:Thrombosis:0.66), (CYAMEMAZINE:Thrombosis:0.07), (DABRAFENIB: Thrombosis:1.38), (DACLATASVIR:Thrombosis:0.5), (DACLIZUMAB:Thrombosis:1.97), (DA NTROLENE SODIUM:Thrombosis:0.21), (DAPAGLIFLOZIN PROPANEDIOLMETFORMIN HYDROCHLORIDE:Thrombosis:0.14), (DEFEROXAMINE MESYLATE:Thrombosis:0.01), (DEGARELIX:Thrombosis:0.11), (DESFLURANE:Thrombosis: 0.29), (DESOXIMETASONE:Thrombosis:0.22), (DEXAMETHASONE ACETATE:Thrombosis:1.45), (DEXAMETHASONE SODIUM PHOSPHATE:Thrombosis:1.23), (DEXMEDETOMIDINE HYDROCHLORIDE:Thrombosis:0.16), (DIFLUNISAL:Thrombosis:0.05), (DIMETHICONESI LICON DIOXIDESIMETHICONE:Thrombosis:0.15), (DOLASETRON MESYLATE:Thrombosis:0.76), (DORZOLAMIDE HYDROCHLORIDE:Thrombosis:0.62), (ELBASVIRGRAZOPREVIR:Thrombosis:0.61), (EMP AGLIFLOZINLINAGLIPTIN:Thrombosis:1.29), (ERIBULIN:Thrombosis:0.68), (ESOMEPRA ZOLE MAGNESIUMNAPROXEN:Thrombosis:0.28), (ESOMEPRAZOLE SODIUM:Thrombosis:0.12), (ESTRADIOLNORETHINDRONE ACETATE:Thrombosis:0.04), (ETHACRYNIC ACID:Thrombosis:0.14), (ETHINYL ESTRADIOLNORETHINDRONE:Thrombosis:0.17), (ETHINYL ESTRADIOLNORGESTREL:Thrombosis:0.16), (ETRETINATE:Thrombosis:0.08), (FERROUS GLYCINE SULFATE:Thrombosis:0.18), (FERROUS SULFATEGLYCINE:Thrombosis:0.36), (FLURAZEPAMFLURAZEPAM HYDROCHLORIDE:Thrombosis:0.02), (FORMOTEROL FUMARATE\MOMETASONE FUROATE:Thrombosis:0.75), (GLIMEPIRIDEROSIGLITAZONE MALEATE:Thrombosis:0.49), (GLUCAGON:Thrombosis:0.28), (GLYCOPYRROLATE:Throm bosis:0.9), (GLYCOSAMINOGLYCANS:Thrombosis:0.2), (GOSERELIN ACETATE:Thrombosis:0), (GUANFACINE HYDROCHLORIDE:Thrombosis:0.09), (HYDROCHLOROTHIAZIDEMETOPROLOL SUCCINATE:Thrombosis:0.29), (HYDROXYCHLOROQUINE DIPHOSPHATE:Thrombosis:1.14), (IBRITUMOMAB TIUXETAN:Thrombosis:0.33), (IDARUBICIN:Thrombosis:0.05), (ILOPROST:Thrombosis:1.18), (IMIGLUCERASE:Thrombosis:0.4), (IMIPENEM:Thrombosis:0.65), (INDOMETHACIN:Thro mbosis:1.25), (INOTUZUMAB OZOGAMICIN:Thrombosis:0.36), (INTERFERON:Thrombosis:1.08), (INTERFERON ALFA-2A:Thrombosis:1.66), (INTERFERON GAMMA-1B:Thrombosis:0.28), (IOPAMIDOL:Thrombosis:0.07), (LACTASE:Thrombosis:0.8), (LANREO TIDE:Thrombosis:0.37), (LEVOLEUCOVORIN CALCIUM:Thrombosis:0.84), (LEVOTHYROXINE SODIUMPOTASSIUM IODIDE:Thrombosis:0.17), (LINSIDOMINE:Thrombosis:0.27), (LOMITAPIDE MESYLATE:Thrombosis:0.98), (LUTETIUM LU-177:Thrombosis:0.2), (MAGNESIUM ASPARTATE HYDROCHLORIDE:Thrombosis:0.42), (MAGNESIUM ASPARTATE\POTASSIUM ASPARTATE:Thrombosis:0.15), (MAGNESIUM GLUCONATE:Thrombosis:0.12), (MEDROXYPROGESTERONE:Thrombosis:0.28), (MEPOLI ZUMAB:Thrombosis:0.7), (MESNA:Thrombosis:0.78), (METFORMIN HYDROCHLORIDEROSIGLITAZONE MALEATE:Thrombosis:0.88), (METFORMIN HYDROCHLORIDESAXAGLIPTIN:Thrombosis:0.21), (METFORMIN PAMOATE:Thrombosis:0.24), (METHENAMINE HIPPURATE:Thrombosis:1.57), (METHENOLONE ENANTHATE:Thrombosis:0.39), (METHOXY POLYETHYLENE GLYCOL-EPOETIN BETA:Thrombosis:0.18), (METRELEPTIN:Thrombosis:0.28), (MEXILETINE:Thrombosis:0.07), (MICAFUNGIN:Thrombosis:1.04), (MIDODRINE HYDROCHLORIDE:Thrombosis:0.7), (MITOXANTRONE HYDROCHLORIDE:Thrombosis:0.02), (MOEXIPRIL HYDROCHLORIDE:Thrombosis:0.64), (NOREPINEPHRINE BITARTRATE:Thrombosis:0.96), (NORETHINDRONE:Thrombosis:1.01), (OLOPATADINE:T hrombosis:0.02), (OXYBUTYNINOXYBUTYNIN CHLORIDE:Thrombosis:0.41), (PARATHYROID HORMONE:Thrombosis:0.02), (PASIREOTIDE:Thrombosis:0.41), (PEGINTERFERON BETA-1A:Thrombosis:1.26), (PENTOSTATIN:Thrombosis:0.7), (PHENAZOPYRIDINE:Thrombosis:0.25), (PHLOROGLUCINOL1,3,5-TRIMETHOXYBENZENE:Thrombosis:0.13), (PILSICAINIDE HYDROCHLORIDE:Thrombosis:0.31), (PIMECROLIMUS:Thrombosis:0.42), (PIRITRAMIDE: Thrombosis:0.12), (PIROXICAM:Thrombosis:1.28), (POLYETHYLENE GLYCOL 3350POTASSIUM CHLORIDE\SODIUM BICARBONATE\SODIUM CHLORIDE\SODIUM SULFATE ANHYDROUS:Thrombosis:0.47), (PONATINIB:Thrombosis:2.62), (PRAZOSIN HYDROCHLORIDE:Thrombosis:0.91), (PREDNISOLONE SODIUM PHOSPHATE:Thrombosis:0.23), (PRISTINAMYCIN:Thrombosis:0.75), (PROTRIPTYLINE HYDROCHLORIDE:Thrombosis:0.21), (PYRAZINAMIDE:Thrombosis:0.78), (QUINACRINE: Thrombosis:0.25), (SARPOGRELATE:Thrombosis:0.12), (SELEXIPAG:Thrombosis:0.68), (SIP ULEUCEL-T:Thrombosis:0.59), (SODIUM CITRATE:Thrombosis:0.31), (SODIUM PHOSPHATE, DIBASIC\SODIUM PHOSPHATE, MONOBASIC:Thrombosis:0.69), (SOFOSBUVIRVELPATASVIR:Thrombosis:0.34), (TAFAM IDIS MEGLUMINE:Thrombosis:0.22), (TEMSIROLIMUS:Thrombosis:0.48), (TERBUTALINE:Thro mbosis:0.84), (TESTOSTERONE ENANTHATE:Thrombosis:0.16), (TESTOSTERONE UNDECANOATE:Thrombosis:0.93), (TETRACYCLINE:Thrombosis:1.42), (TOCOPHEROL:T hrombosis:1.15), (TRAMETINIB:Thrombosis:1.25), (TRIMIPRAMINE MALEATE:Thrombosis:0.46), (TRIPTORELIN PAMOATE:Thrombosis:0.57), (UMECLIDINIUM BROMIDE:Thrombosis:1.02), (VANDETANIB:Thrombosis:0.02), (VARDENAFIL HYDROCHLORIDE TRIHYDRATE:Thrombosis:0.66), (VASOPRESSIN:Thrombosis:0.36), (YEAST:Thrombosis:0.33), (ZILEUTON:Thrombosis:0.36), (.ALPHA.1-PROTEINASE INHIBITOR HUMAN:Thrombosis prophylaxis:1.58), (.BETA.-CAROTENE:Thrombosis prophylaxis:0.3), (.BETA.-HYDROXYISOVALERIC ACIDARGININEGLUTAMINE:Thrombosis prophylaxis:0.51), (ABOBOTULINUMTOXINA:Thrombosis prophylaxis:0.84), (ACAMPROSATE:Thrombosis prophylaxis:0.36), (ACETAMINOPHEN\BELLADONNA LEAFCAFFEINE\MORPHINE:Thrombosis prophylaxis:0.26), (ACETAMINOPHENDICHLORALPHENAZONEISOMETHEPTENE:Thr ombosis prophylaxis:0.08), (ACETAMINOPHENDIPHENHYDRAMINE HYDROCHLORIDE:Thrombosis prophylaxis:0.8), (ACETAMINOPHENMETHOCARBAMOL:Thrombosis prophylaxis:0.69), (ACETIC ACIDSODIUM ACETATE:Thrombosis prophylaxis:0.51), (ADENINE:Thrombosis prophylaxis:0.92), (ADUCANUMAB:Thrombosis prophylaxis:0.28), (ALBUMIN BOVINETANNIC ACID:Thrombosis prophylaxis:0.99), (ALBUTEROLGUAIFENESIN:Thrombosis prophylaxis:0.29), (ALDESLEUKIN:Thrombosis prophylaxis:1.07), (ALDIOXA:Thrombosis prophylaxis:0.01), (ALISKIREN HEMIFUMARATEHYDROCHLOROTHIAZIDE:Thrombosis prophylaxis:0.22), (ALPROSTADIL ALFADEX:Thrombosis prophylaxis:0.22), (ALUMINUM HYDROXIDEMAGNESIUM OXIDEOXETHAZAINE:Thrombosis prophylaxis:1.15), (AMANTADINE SULFATE:Thrombosis prophylaxis:0.05), (AMINOSALICYLIC ACID:Thrombosis prophylaxis:0.1), (AMLODIPINE BESYLATEAZILSARTAN:Thrombosis prophylaxis:0.66), (AMMONIUM GLYCYRRHIZATECYSTEINE HYDROCHLORIDEGLYCINE:Thrombosis prophylaxis:0.41), (AMOXAPINE:Thrombosis prophylaxis:1.06), (AMOXICILLINCLARITHROMYCINESOMEPRAZOLE:Thrombosis prophylaxis:1. 77), (AMOXICILLINCLARITHROMYCINLANSOPRAZOLE:Thrombosis prophylaxis:0.16), (ANAGLIPTIN:Thrombosis prophylaxis:1.64), (APOMORPHINE:Thrombosis prophylaxis:0.02), (ARFORMOTEROL:Thrombosis prophylaxis:0.06), (ARIPIPRAZOLE LAUROXIL:Thrombosis prophylaxis:0.09), (AROTINOLOL:Thrombosis prophylaxis:0.76), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE MONONITRATE:Thrombosis prophylaxis:0.52), (ASCORBIC ACIDCALCIUM PANTOTHENATE:Thrombosis prophylaxis:0.52), (ASCORBIC ACIDPOLYETHYLENE GLYCOL 3350POTASSIUM CHLORIDE\SODIUM ASCORBATE\SODIUM CHLORIDE\SODIUM SULFATE:Thrombosis prophylaxis:0.5), (ASUNAPREVIR:Thrombosis prophylaxis:1.32), (ATAZANAVIR:Thrombosis prophylaxis:0.4), (AVOBENZONEHOMOSALATEOCTISALATE:Thrombosis prophylaxis:0.82), (AZITHROMYCIN MONOHYDRATE:Thrombosis prophylaxis:0.46), (AZITHROMYCIN OR METHOTREXATE SODIUM:Thrombosis prophylaxis:1.7), (BACITRACINNEOMYCIN SULFATE:Thrombosis prophylaxis:0.1), (BALSALAZIDE:Thrombosis prophylaxis:0.48), (BEPOTASTINE BESILATE:Thrombosis prophylaxis:0.24), (BERAPROST:Thrombosis prophylaxis:1.05), (BETAMETHASONE DIPROPIONATECALCIPOTRIENE HYDRATE:Thrombosis prophylaxis:0.03), (BIFIDOBACTERIUM ANIMALIS SUBSP. LACTIS:Thrombosis prophylaxis:0.37), (BIFIDOBACTERIUM LONGUM:Thrombosis prophylaxis:0.62), (BIFIDOBACTERIUM LONGUM\LACTOBACILLUS ACIDOPHILUS:Thrombosis prophylaxis:0.02), (BIFIDOBACTERIUM LONGUM\LACTOBACILLUS ACIDOPHILUS\LACTOBACILLUS RHAMNOSUS:Thrombosis prophylaxis:0.01), (BIMATOPROSTTIMOLOL:Thrombosis prophylaxis:1.22), (BIXALOMER:Thrombosis prophylaxis:0.75), (BLESELUMAB:Thrombosis prophylaxis:0.65), (BOTULINUM TOXIN TYPE A:Thrombosis prophylaxis:1.13), (BOTULISM ANTITOXIN:Thrombosis prophylaxis:0.25), (BROMHEXINE:Thrombosis prophylaxis:0.98), (BUFORMIN:Thrombosis prophylaxis:0.67), (BUPIVACAINEEPINEPHRINE:Thrombosis prophylaxis:0.14), (BUTYLSCOPOLAMINE:Thrombosis prophylaxis:0.3), (CABOZANTINIB:Thrombosis prophylaxis:0.82), (CAFFEINEDIPYRONEORPHENADRINE:Thrombosis prophylaxis:0.08), (CAJUPUT OIL\EUCALYPTUS OILPEPPERMINT OIL:Thrombosis prophylaxis:0), (CALCIUM ACETATEMAGNESIUM CARBONATE:Thrombosis prophylaxis:0.43), (CALCIUM BICARBONATE:Thrombosis prophylaxis:0.25), (CALCIUM LACTATE:Thrombosis prophylaxis:1.6), (CALCIUM PANTOTHENATECYANOCOBALAMINNIACINAMIDEPYRIDOXINE HYDROCHLORIDE\RIBOFLAVINTHIAMINE MONONITRATE:Thrombosis prophylaxis:0.82), (CALCIUM PHOSPHATE:Thrombosis prophylaxis:0.41), (CALCIUM SULFATEHERBALS:Thrombosis prophylaxis:0.04), (CAMPHOR\CAPSICUM\METHYL SALICYLATE:Thrombosis prophylaxis:0.13), (CAMPHORDIPHENHYDRAMINE\ZINC OXIDE:Thrombosis prophylaxis:0.46), (CANAGLIFLOZINMETFORMIN HYDROCHLORIDE:Thrombosis prophylaxis:2.24), (CANGRELOR:Thrombosis prophylaxis:0.19), (CARBON DIOXIDE:Thrombosis prophylaxis:0.01), (CEFCAPENE PIVOXIL:Thrombosis prophylaxis:0.04), (CEFMETAZOLE:Thrombosis prophylaxis:0.01), (CEFMETAZOLE SODIUM:Thrombosis prophylaxis:0.72), (CEFPROZIL:Thrombosis prophylaxis:0.46), (CEPHARANTHINE:Thrombosis prophylaxis:0.61), (CERIVASTATIN SODIUM:Thrombosis prophylaxis:0.3), (CHLOROTHIAZIDE:Thrombosis prophylaxis:1.42), (CHLORPHENIRAMINE MALEATDIHYDROCODEINE PHOSPHATEMETHYLEPHEDRINE HYDROCHLORIDE, DL-:Thrombosis prophylaxis:0.39), (CHLORZOXAZONE:Thrombosis prophylaxis:0.1), (CHOLESTEROL:Thrombosis prophylaxis:0.3), (CHOLINE BITARTRATECYSTEINEINOSITOLMAMMAL LIVER:Thrombosis prophylaxis:0.37), (CHROMIC CHLORIDECHROMIUM:Thrombosis prophylaxis:0.16), (CICLOPIROX OLAMINE:Thrombosis prophylaxis:0.64), (CIPROFLOXACINDEXAMETHASONE:Thrombosis prophylaxis:0.1), (CISAPRIDE:Thrombosis prophylaxis:0.49), (CLADRIBINE:Thrombosis prophylaxis:0.24), (CLIKSTAR:Thrombosis prophylaxis:0.86), (CLOXAZOLAM:Thrombosis prophylaxis:0.84), (COBICISTATELVITEGRAVIREMTRICITABINETENOFOVIR ALAFENAMIDE FUMARATE:Thrombosis prophylaxis:0.02), (CROTALIDAE POLYVALENT IMMUNE FAB (OVINE):Thrombosis prophylaxis:0.77), (CROTAMITON:Thrombosis prophylaxis:0.44), (CUPRIC SULFATEFERRIC CHLORIDEMANGANESE CHLORIDE\POTASSIUM IODIDE\ZINC SULFATE:Thrombosis prophylaxis:0.07), (CYANAMIDE:Thrombosis prophylaxis:0.12), (CYANOCOBALAMINPYRIDOXINE HYDROCHLORIDETHIAMINE DISULFIDE:Thrombosis prophylaxis:0.26), (CYPROTERONE:Thrombosis prophylaxis:0.22), (DAPAGLIFLOZIN PROPANEDIOLMETFORMIN HYDROCHLORIDE:Thrombosis prophylaxis:0.84), (DEMECLOCYCLINE:Thrombosis prophylaxis:0.11), (DESFLURANE:Thrombosis prophylaxis:0.94), (DESLANOSIDE:Thrombosis prophylaxis:0.35), (DEXTROAMPHETAMINE:Thrombosis prophylaxis:0.38), (DEXTROAMPHETAMINE SULFATE:Thrombosis prophylaxis:1.13), (DIAZOXIDE:Thrombosis prophylaxis:0.44), (DICLOFENAC DIETHYLAMINE:Thrombosis prophylaxis:0.06), (DIGITALIS:Thrombosis prophylaxis:1), (DILAZEP:Thrombosis prophylaxis:1.49), (DIPYRONEPROMETHAZINE HYDROCHLORIDE:Thrombosis prophylaxis:0.73), (DOLASETRON MESYLATE:Thrombosis prophylaxis:1.56), (DOLUTEGRAVIR:Thrombosis prophylaxis:0.44), (DOXERCALCIFEROL:Thrombosis prophylaxis:0.28), (DROSPIRENONEETHINYL ESTRADIOLLEVOMEFOLATE CALCIUM:Thrombosis prophylaxis:0.28), (DYDROGESTERONE:Thrombosis prophylaxis:0.59), (ECABET SODIUM:Thrombosis prophylaxis:1.25), (ECALLANTIDE:Thrombosis prophylaxis:0.1), (EFAVIRENZEMTRICITABINETENOFOVIR DISOPROXIL FUMARATE:Thrombosis prophylaxis:0.41), (ELBASVIRGRAZOPREVIR:Thrombosis prophylaxis:0.13), (ELTROMBOPAG OLAMINE:Thrombosis prophylaxis:0.94), (EMPAGLIFLOZINMETFORMIN HYDROCHLORIDE:Thrombosis prophylaxis:0.08), (EMTRICITABINE:Thrombosis prophylaxis:0.35), (EMTRICITABINETENOFOVIR:Thrombosis prophylaxis:0.14), (EPACADOSTAT:Thrombosis prophylaxis:0.44), (EPHEDRA SINICA STEMHERBALS:Thrombosis prophylaxis:0.23), (EPRAZINONE:Thrombosis prophylaxis:0.13), (ERGOLOID MESYLATES:Thrombosis prophylaxis:0.04), (ESTRIOL:Thrombosis prophylaxis:0.99), (ETHINYL ESTRADIOLNORGESTREL:Thrombosis prophylaxis:0.77), (ETHIODIZED OIL:Thrombosis prophylaxis:0.3), (ETIFOXINE:Thrombosis prophylaxis:0.15), (ETIMICIN SULFATE:Thrombosis prophylaxis:0.54), (ETRETINATE:Thrombosis prophylaxis:2.06), (FELODIPINEMETOPROLOL SUCCINATE:Thrombosis prophylaxis:0.57), (FERRIC SODIUM CITRATE:Thrombosis prophylaxis:0.34), (FERROUS BISGLYCINATE:Thrombosis prophylaxis:1.04), (FLAVIN ADENINE DINUCLEOTIDE:Thrombosis prophylaxis:0.93), (FLAVOXATE HYDROCHLORIDE:Thrombosis prophylaxis:0.34), (FLOMOXEF:Thrombosis prophylaxis:0.44), (FLOMOXEF SODIUM:Thrombosis prophylaxis:0.04), (FLUOROURACILLEUCOVORIN CALCIUMOXALIPLATIN:Thrombosis prophylaxis:0.5), (FLUPHENAZINE:Thrombosis prophylaxis:0.2), (FORMOTEROL FUMARATE\MOMETASONE FUROATE:Thrombosis prophylaxis:1.76), (FOSFLUCONAZOLE:Thrombosis prophylaxis:0.44), (FOSFOMYCIN CALCIUM:Thrombosis prophylaxis:0.23), (FOSPHENYTOINFOSPHENYTOIN SODIUM:Thrombosis prophylaxis:1.34), (FROVATRIPTAN SUCCINATE:Thrombosis prophylaxis:0.15), (FUDOSTEINE:Thrombosis prophylaxis:0.86), (FUROSEMIDE SODIUM:Thrombosis prophylaxis:0.05), (FUSIDATE SODIUM:Thrombosis prophylaxis:0.38), (GABEXATE MESYLATE:Thrombosis prophylaxis:0.82), (GARENOXACIN:Thrombosis prophylaxis:0.14), (GLIMEPIRIDEROSIGLITAZONE MALEATE:Thrombosis prophylaxis:0.93), (GLIPIZIDEMETFORMIN HYDROCHLORIDE:Thrombosis prophylaxis:0.67), (GLYCOPYRROLATEINDACATEROL MALEATE:Thrombosis prophylaxis:1.45), (GLYCOPYRRONIUMFORMOTEROL FUMARATE:Thrombosis prophylaxis:0.07), (GUSPERIMUS TRIHYDROCHLORIDE:Thrombosis prophylaxis:0.1), (HALOPERIDOL DECANOATE:Thrombosis prophylaxis:0.1), (HOMATROPINE METHYLBROMIDEHYDROCODONE BITARTRATE:Thrombosis prophylaxis:0.05), (HORSE CHESTNUT:Thrombosis prophylaxis:0.19), (HYDROCHLOROTHIAZIDEMETOPROLOL TARTRATE:Thrombosis prophylaxis:0.23), (HYDROCORTISONE SODIUM PHOSPHATE:Thrombosis prophylaxis:0.17), (HYOSCYAMINE SULFATEMETHENAMINEMETHYLENE BLUEPHENYL SALICYLATE\SODIUM PHOSPHATE, MONOBASIC, MONOHYDRATE:Thrombosis prophylaxis:0.2), (IBRITUMOMAB TIUXETAN:Thrombosis prophylaxis:1.16), (IFENPRODIL TARTRATE:Thrombosis prophylaxis:0.34), (IGURATIMOD:Thrombosis prophylaxis:1.16), (IMIDAFENACIN:Thrombosis prophylaxis:0.64), (IMIGLUCERASE:Thrombosis prophylaxis:1.41), (INDACATEROL MALEATE:Thrombosis prophylaxis:0.21), (INFLIXIMAB-DYYB:Thrombosis prophylaxis:1.33), (INOTUZUMAB OZOGAMICIN:Thrombosis prophylaxis:1.3), (INSULIN ASPARTINSULIN DEGLUDEC:Thrombosis prophylaxis:1.24), (INSULIN PORKANSULIN PURIFIED PORK:Thrombosis prophylaxis:1), (INTERFERON:Thrombosis prophylaxis:2), (INTERFERON ALFA-2A:Thrombosis prophylaxis:2.09), (INTERFERON ALFA-N1:Thrombosis prophylaxis:0.15), (INTERFERON BETA:Thrombosis prophylaxis:0.25), (INTERFERON GAMMA-1B:Thrombosis prophylaxis:0.69), (IPRAGLIFLOZIN:Thrombosis prophylaxis:0.16), (ISAVUCONAZOLE:Thrombosis prophylaxis:0.04), (ITRACONAZOLE\SODIUM CHLORIDE:Thrombosis prophylaxis:0.41), (IXABEPILONE:Thrombosis prophylaxis:0.46), (LACTASE:Thrombosis prophylaxis:1.44), (LEUPROLIDE:Thrombosis prophylaxis:0.25), (LEVOFLOXACIN HYDROCHLORIDE:Thrombosis prophylaxis:0.17), (LICORICE ROOT EXTRACTRHUBARB:Thrombosis prophylaxis:0.14), (LOMUSTINE:Thrombosis prophylaxis:0.22), (LUTETIUM LU-177:Thrombosis prophylaxis:0.67), (LYSOZYME HYDROCHLORIDE:Thrombosis prophylaxis:0.95), (MALTOSE:Thrombosis prophylaxis:0), (MENTHOLZINC OXIDE:Thrombosis prophylaxis:0.36), (MEPENZOLATE BROMIDE:Thrombosis prophylaxis:0.71), (MEQUITAZINE:Thrombosis prophylaxis:0.09), (METFORMIN HYDROCHLORIDEROSIGLITAZONE MALEATE:Thrombosis prophylaxis:0.99), (METFORMIN HYDROCHLORIDESAXAGLIPTIN:Thrombosis prophylaxis:1.11), (METHENOLONE ACETATE:Thrombosis prophylaxis:0.41), (METHENOLONE ENANTHATE:Thrombosis prophylaxis:2.41), (METHYLENE BLUE:Thrombosis prophylaxis:0.93), (METHYLNALTREXONE:Thrombosis prophylaxis:0.14), (MIGLUSTAT:Thrombosis prophylaxis:0.32), (MILRINONE LACTATE:Thrombosis prophylaxis:0.9), (MIZORIBINE:Thrombosis prophylaxis:1.96), (NADIFLOXACIN:Thrombosis prophylaxis:0.26), (NAFAMOSTAT:Thrombosis prophylaxis:0.16), (NAFRONYL:Thrombosis prophylaxis:0.2), (NALDEMEDINE:Thrombosis prophylaxis:0.09), (NALFURAFINE HYDROCHLORIDE:Thrombosis prophylaxis:0.32), (NEFAZODONE:Thrombosis prophylaxis:0), (NEFAZODONE HYDROCHLORIDE:Thrombosis prophylaxis:0.29), (NIFUROXAZIDE:Thrombosis prophylaxis:0.07), (OK-432:Thrombosis prophylaxis:0.16), (OLOPATADINE:Thrombosis prophylaxis:1.2), (OXETORONE:Thrombosis prophylaxis:0.01), (PANCURONIUM:Thrombosis prophylaxis:0.7), (PANTHENOL:Thrombosis prophylaxis:0.1), (PAZUFLOXACIN MESILATE:Thrombosis prophylaxis:0.6), (PEGINTERFERON ALFA-2A OR PEGINTERFERON ALFA-2B:Thrombosis prophylaxis:0.16), (PEGVISOMANT:Thrombosis prophylaxis:0.66), (PENICILLAMINE:Thrombosis prophylaxis:0.27), (PENTAERYTHRITOL TETRANITRATE:Thrombosis prophylaxis:0.71), (PENTOBARBITAL:Thrombosis prophylaxis:0.1), (PENTOSTATIN:Thrombosis prophylaxis:1.47), (PERAMIVIR:Thrombosis prophylaxis:0.28), (PEROSPIRONE HYDROCHLORIDE:Thrombosis prophylaxis:0.01), (PHENAZOPYRIDINE HYDROCHLORIDE:Thrombosis prophylaxis:0.81), (PHENOTHIAZINE:Thrombosis prophylaxis:0.22), (PHENYLBUTAZONE:Thrombosis prophylaxis:0.03), (PHENYLEPHRINE HYDROCHLORIDEWITCH HAZEL:Thrombosis prophylaxis:0.18), (PIRENZEPINE:Thrombosis prophylaxis:0.55), (PIRETANIDE:Thrombosis prophylaxis:0.49), (PITOFENONE:Thrombosis prophylaxis:0.46), (PLASMA PROTEIN FRACTION (HUMAN):Thrombosis prophylaxis:0.25), (POLYCARBOPHIL:Thrombosis prophylaxis:0.25), (POLYETHYLENE GLYCOL 400:Thrombosis prophylaxis:0.15), (POLYMYXIN B SULFATETRIMETHOPRIM SULFATE:Thrombosis prophylaxis:0.17), (POLYSTYRENE SULFONIC ACID:Thrombosis prophylaxis:1.27), (POLYVINYL ALCOHOLPOVIDONE:Thrombosis prophylaxis:0.02), (PORFIMER SODIUM:Thrombosis prophylaxis:1.05), (POTASSIUM PERCHLORATE:Thrombosis prophylaxis:0.11), (POVIDONE:Thrombosis prophylaxis:1.09), (PRAMLINTIDE ACETATE:Thrombosis prophylaxis:0.11), (PREDNISOLONE SODIUM METAZOATE:Thrombosis prophylaxis:0.05), (PREDNISOLONE SODIUM PHOSPHATE:Thrombosis prophylaxis:0.93), (PREDNISOLONE VALERATE ACETATE:Thrombosis prophylaxis:0.37), (PROCYCLIDINE:Thrombosis prophylaxis:0.18), (PROMAZINE:Thrombosis prophylaxis:0.01), (PROPATYL NITRATE:Thrombosis prophylaxis:0.69), (PROTRIPTYLINE HYDROCHLORIDE:Thrombosis prophylaxis:0.53), (PYRIDOXAL PHOSPHATE:Thrombosis prophylaxis:0.94), (PYRIMETHAMINE:Thrombosis prophylaxis:0.5), (QUAZEPAM:Thrombosis prophylaxis:0.47), (QUINACRINE:Thrombosis prophylaxis:1.14), (REGADENOSON:Thrombosis prophylaxis:0.19), (RETEPLASE:Thrombosis prophylaxis:0.69), (RHUBARB:Thrombosis prophylaxis:0.56), (RIBOCICLIB:Thrombosis prophylaxis:0.16), (RILPIVIRINE HYDROCHLORIDE:Thrombosis prophylaxis:1.1), (RISEDRONIC ACID:Thrombosis prophylaxis:0.67), (RIZATRIPTAN:Thrombosis prophylaxis:1), (ROFECOXIB:Thrombosis prophylaxis:0.39), (ROSIGLITAZONE:Thrombosis prophylaxis:0.41), (SARGRAMOSTIM:Thrombosis prophylaxis:0.17), (SELEXIPAG:Thrombosis prophylaxis:0.6), (SENNOSIDE B:Thrombosis prophylaxis:0.93), (SIBUTRAMINE HYDROCHLORIDE:Thrombosis prophylaxis:0.77), (SITAFLOXACIN:Thrombosis prophylaxis:0.58), (SODIUM BICARBONATE\SODIUM CHLORIDE:Thrombosis prophylaxis:0.13), (SODIUM PHOSPHATE, DIBASIC:Thrombosis prophylaxis:0.53), (SULTOPRIDE HYDROCHLORIDE:Thrombosis prophylaxis:0.1), (SUNITINIB:Thrombosis prophylaxis:0.29), (SUPLATAST TOSILATE:Thrombosis prophylaxis:1.68), (TABALUMAB:Thrombosis prophylaxis:0.48), (TANDOSPIRONE:Thrombosis prophylaxis:0.03), (TASIMELTEON:Thrombosis prophylaxis:0.06), (TELOTRISTAT ETHYL:Thrombosis prophylaxis:0.47), (TENECTEPLASE:Thrombosis prophylaxis:1.53), (TENOXICAM:Thrombosis prophylaxis:0.18), (THIAMYLAL SODIUM:Thrombosis prophylaxis:0.8), (TIAPROFENIC ACID:Thrombosis prophylaxis:0.05), (TIBOLONE:Thrombosis prophylaxis:0.55), (TIMOLOL MALEATETRAVOPROST:Thrombosis prophylaxis:1.63), (TIMOLOLTRAVOPROST:Thrombosis prophylaxis:0.66), (TIQUIZIUM BROMIDE:Thrombosis prophylaxis:0.24), (TIROFIBAN:Thrombosis prophylaxis:0.79), (TIROFIBAN HYDROCHLORIDE:Thrombosis prophylaxis:0.87), (TOFACITINIB:Thrombosis prophylaxis:0.07), (TOFISOPAM:Thrombosis prophylaxis:0.14), (TOPIROXOSTAT:Thrombosis prophylaxis:0.27), (TREPROSTINIL DIOLAMINE:Thrombosis prophylaxis:0.28), (VALDECOXIB:Thrombosis prophylaxis:1), (VECURONIUM BROMIDE:Thrombosis prophylaxis:1.36), (VINBLASTINE SULFATE:Thrombosis prophylaxis:0.33), (YEAST:Thrombosis prophylaxis:1.01), (ZICONOTIDE ACETATE:Thrombosis prophylaxis:1.02), (ZILEUTON:Thrombosis prophylaxis:1.21), (ZIV-AFLIBERCEPT:Thrombosis prophylaxis:1.51), (.ALPHA.1-PROTEINASE INHIBITOR HUMAN:Thyroid cancer:0.2), (ABIRATERONE ACETATE:Thyroid cancer:0.19), (ACARBOSE:Thyroid cancer:0.49), (ACETAMINOPHENOXYCODONEOXYCODONE HYDROCHLORIDE:Thyroid cancer:0.3), (ACETAMINOPHENTRAMADOL:Thyroid cancer:0.05), (ACETAZOLAMIDE:Thyroid cancer:0.51), (ADO-TRASTUZUMAB EMTANSINE:Thyroid cancer:0.56), (ALENDRONIC ACID:Thyroid cancer:0.15), (APREPITANT:Thyroid cancer:0.1), (BISACODYL:Thyroid cancer:0.49), (BORTEZOMIB:Thyroid cancer:0.01), (BUPRENORPHINE HYDROCHLORIDE:Thyroid cancer:0.3), (CANAGLIFLOZINMETFORMIN HYDROCHLORIDE:Thyroid cancer:0.31), (CARFILZOMIB:Thyroid cancer:0.42), (CARVEDILOL PHOSPHATE:Thyroid cancer:0.83), (CERTOLIZUMAB PEGOL:Thyroid cancer:1.15), (CHLORDIAZEPOXIDE HYDROCHLORIDECLIDINIUM BROMIDE:Thyroid cancer:0.02), (CLARITHROMYCIN:Thyroid cancer:0.03), (CLOZAPINE:Thyroid cancer:0.39), (CORTICOTROPIN:Thyroid cancer:0.05), (CRIZOTINIB:Thyroid cancer:0.04), (DACLIZUMAB:Thyroid cancer:0.29), (DALFAMPRIDINE:Thyroid cancer:0.05), (DAPAGLIFLOZIN PROPANEDIOL:Thyroid cancer:0.33), (DARBEPOETIN ALFA:Thyroid cancer:0.06), (DASATINIB:Thyroid cancer:1.12), (DESONIDE:Thyroid cancer:0.02), (DEVICE:Thyroid cancer:0.42), (DIMENHYDRINATE:Thyroid cancer:0.03), (DOXEPIN HYDROCHLORIDE:Thyroid cancer:0.27), (EDOXABAN TOSYLATE:Thyroid cancer:0.31), (EMPAGLIFLOZINLINAGLIPTIN:Thyroid cancer:0.21), (ERYTHROMYCIN:Thyroid cancer:0.1), (ESTROGENS, CONJUGATEDMEDROXYPROGESTERONE ACETATE:Thyroid cancer:0.68), (EVOLOCUMAB:Thyroid cancer:1.15), (FEXOFENADINEPSEUDOEPHEDRINE:Thyroid cancer:0.05), (FLUTICASONE FUROATE:Thyroid cancer:0.38), (FLUTICASONE FUROATESALMETEROL XINAFOATE:Thyroid cancer:0.3), (GLYBURIDE:Thyroid cancer:0.54), (GLYCOPYRRONIUMINDACATEROL:Thyroid cancer:0.21), (HEPARIN SODIUM:Thyroid cancer:0.33), (HERBALSTURMERIC:Thyroid cancer:0.55), (HYDROCHLOROTHIAZIDETELMISARTAN:Thyroid cancer:0.23), (HYOSCYAMINE:Thyroid cancer:0.35), (ILOPROST:Thyroid cancer:0.35), (IMATINIB MESYLATE:Thyroid cancer:0.18), (IMIPRAMINE HYDROCHLORIDE:Thyroid cancer:0.53), (INDAPAMIDE:Thyroid cancer:0.59), (INDAPAMIDEPERINDOPRIL:Thyroid cancer:0.56), (IPRATROPIUM:Thyroid cancer:0.83), (LACOSAMIDE:Thyroid cancer:0), (LACTASE:Thyroid cancer:0.15), (LEDIPASVIRSOFOSBUVIR:Thyroid cancer:0.52), (LERCANIDIPINE HYDROCHLORIDE:Thyroid cancer:0.51), (LIDOCAINEPRILOCAINE:Thyroid cancer:0.54), (LISDEXAMFETAMINE DIMESYLATE:Thyroid cancer:0.52), (LITHIUM CARBONATE:Thyroid cancer:0.87), (LOMITAPIDE MESYLATE:Thyroid cancer:0.15), (MECLIZINEMECLIZINE HYDROCHLORIDE:Thyroid cancer:0.11), (MEPOLIZUMAB:Thyroid cancer:0.15), (METFORMIN HYDROCHLORIDEVILDAGLIPTIN:Thyroid cancer:0.01), (METHENAMINE HIPPURATE:Thyroid cancer:0.36), (METHYLPREDNISOLONE ACETATE:Thyroid cancer:0.38), (MIRABEGRON:Thyroid cancer:0.22), (MOXIFLOXACIN HYDROCHLORIDE:Thyroid cancer:1.07), (NATEGLINIDE:Thyroid cancer:0.14), (PAMIDRONATE DISODIUM:Thyroid cancer:0.18), (PEGINTERFERON BETA-1A:Thyroid cancer:0.09), (PEMETREXED DISODIUM:Thyroid cancer:0.16), (PHYTONADIONE:Thyroid cancer:0.09), (PIROXICAM:Thyroid cancer:0.12), (PONATINIB:Thyroid cancer:0.32), (PRIMIDONE:Thyroid cancer:1.22), (PROGESTERONE:Thyroid cancer:0.42), (RIZATRIPTAN BENZOATE:Thyroid cancer:0.53), (ROFLUMILAST:Thyroid cancer:0.6), (ROPINIROLE:Thyroid cancer:0.01), (SACUBITRILVALSARTAN:Thyroid cancer:0.39), (SCOPOLAMINE:Thyroid cancer:0.41), (SECUKINUMAB:Thyroid cancer:0.38), (SUMATRIPTAN:Thyroid cancer:0.2), (SUMATRIPTANSUMATRIPTAN SUCCINATE:Thyroid cancer:0.61), (TERBINAFINE HYDROCHLORIDE:Thyroid cancer:0.09), (TERBUTALINE SULFATE:Thyroid cancer:0.28), (TERIFLUNOMIDE:Thyroid cancer:0.45), (TETRACYCLINE:Thyroid cancer:0.58), (THALIDOMIDE:Thyroid cancer:0.26), (TOCILIZUMAB:Thyroid cancer:0.51), (UMECLIDINIUM BROMIDEVILANTEROL TRIFENATATE:Thyroid cancer:0.9), (VINORELBINE TARTRATE:Thyroid cancer:0.06), (ACECLOFENAC:Thyroid disorder:0.22), (ACETAMINOPHEN\CAFFEINE:Thyroid disorder:0.97), (ACETAMINOPHENCHLORPHENIRAMINE MALEATEPSEUDOEPHEDRINE HYDROCHLORIDE:Thyroid disorder:0.63), (ACETAMINOPHENMETHOCARBAMOL:Thyroid disorder:0.95), (ACTIVATED CHARCOAL:Thyroid disorder:0.14), (ADO-TRASTUZUMAB EMTANSINE:Thyroid disorder:1.48), (ALFALFA:Thyroid disorder:0.14), (ALISKIREN HEMIFUMARATEHYDROCHLOROTHIAZIDE:Thyroid disorder:0.6), (AMBROXOL:Thyroid disorder:0.11), (AMILORIDE HYDROCHLORIDEFUROSEMIDE:Thyroid disorder:0.24), (AMINO ACIDSDEXTROSEELECTROLYTES NOS:Thyroid disorder:0.9), (AMLODIPINEPERINDOPRIL:Thyroid disorder:0.24), (AMMONIUM CHLORIDEDEXTROMETHORPHAN\SODIUM CITRATE:Thyroid disorder:0.78), (AMPICILLIN SODIUMSULBACTAM SODIUM:Thyroid disorder:0.09), (ANAKINRA:Thyroid disorder:0.35), (ARIPIPRAZOLE LAUROXIL:Thyroid disorder:1.44), (ASCORBIC ACID/CHROMIUM/SELENIUM/VANADYL SULFATE:Thyroid disorder:0.66), (ASCORBIC ACIDBIOTINCYANOCOBALAMINFOLIC ACIDNIACINPANTOTHENIC ACIDPYRIDOXINE\RIBOFLAVINTHIAMINE:Thyroid disorder:0.02), (ASPIRIN\CAFFEINESALICYLAMIDE:Thyroid disorder:0.31), (ASPIRINMAGNESIUM OXIDE:Thyroid disorder:0.14), (AVANAFIL:Thyroid disorder:0.48), (BECLOMETHASONE:Thyroid disorder:0.51), (BETAMETHASONE DIPROPIONATECALCIPOTRIENE:Thyroid disorder:0.68), (BETAMETHASONECALCIPOTRIENE:Thyroid disorder:1.27), (BIMATOPROSTTIMOLOL:Thyroid disorder:0.35), (BISMUTH SUBSALICYLATEGLYCERINPROPYLENE GLYCOL:Thyroid disorder:0.22), (BLINATUMOMAB:Thyroid disorder:0.18), (BOTULINUM TOXIN TYPE A:Thyroid disorder:1.22), (BROTIZOLAM:Thyroid disorder:0.23), (BUPROPION HYDROBROMIDE:Thyroid disorder:0.03), (BUTORPHANOL:Thyroid disorder:0.05), (CALCIUM BICARBONATE:Thyroid disorder:0.05), (CALCIUM CARBONATEERGOCALCIFEROL:Thyroid disorder:0.09), (CALCIUM CARBONATEERGOCALCIFEROLRETINOL:Thyroid disorder:0.7), (CALCIUM PANTOTHENATECYANOCOBALAMINNIACINAMIDEPYRIDOXINE HYDROCHLORIDE\RIBOFLAVINTHIAMINE HYDROCHLORIDE:Thyroid disorder:0.01), (CALCIUM PANTOTHENATECYANOCOBALAMINNIACINAMIDEPYRIDOXINE HYDROCHLORIDE\RIBOFLAVINTHIAMINE MONONITRATE:Thyroid disorder:0.38), (CANNABIDIOL:Thyroid disorder:0.19), (CARBAMIDE PEROXIDE:Thyroid disorder:0.49), (CARBOXYMETHYLCELLULOSE SODIUMHYPROMELLOSES:Thyroid disorder:0.18), (CEFAZOLIN:Thyroid disorder:0.63), (CEFOTAXIME SODIUM:Thyroid disorder:0.19), (CERITINIB:Thyroid disorder:0.38), (CERIVASTATIN SODIUM:Thyroid disorder:1.19), (CHLORAMBUCIL:Thyroid disorder:0.13), (CICLOPIROX OLAMINE:Thyroid disorder:0.13), (CILASTATIN SODIUMIMIPENEM:Thyroid disorder:0.44), (CODEINE PHOSPHATEPROMETHAZINE:Thyroid disorder:0.02), (CODEINE SULFATE:Thyroid disorder:0.02), (CYPROHEPTADINE HYDROCHLORIDE:Thyroid disorder:0.51), (DABRAFENIB MESYLATE:Thyroid disorder:0.49), (DANTROLENE SODIUM:Thyroid disorder:0.57), (DESVENLAFAXINE:Thyroid disorder:0.1), (DEXAMETHASONE ACETATE:Thyroid disorder:0.02), (DEXAMETHASONENEOMYCINPOLYMYXIN B SULFATE:Thyroid disorder:0.18), (DEXMETHYLPHENIDATE HYDROCHLORIDE:Thyroid disorder:0.4), (DEXTRAN 70HYPROMELLOSE 2910 (4000 MPA.S):Thyroid disorder:0.15), (DICHLORPHENAMIDE:Thyroid disorder:0.11), (DICLOFENAC DIETHYLAMINE:Thyroid disorder:0.02), (DIETARY SUPPLEMENTRESVERATROL:Thyroid disorder:0.06), (DIFLUNISAL:Thyroid disorder:0.98), (DIMETHICONESILICON DIOXIDESIMETHICONE:Thyroid disorder:0.15), (DIMETHYL SULFOXIDE:Thyroid disorder:0.09), (DOLASETRON MESYLATE:Thyroid disorder:0.81), (DOXYLAMINE SUCCINATEPYRIDOXINE HYDROCHLORIDE:Thyroid disorder:0.06), (DROSPIRENONEESTRADIOL:Thyroid disorder:0.09), (DROSPIRENONEETHINYL ESTRADIOLLEVOMEFOLATE CALCIUM:Thyroid disorder:0.47), (ECONAZOLE:Thyroid disorder:1.01), (ELOTUZUMAB:Thyroid disorder:0.38), (ENALAPRIL MALEATELERCANIDIPINE HYDROCHLORIDE:Thyroid disorder:0.06), (ENFUVIRTIDE:Thyroid disorder:0.03), (ENSULIZOLEEUCERINOCTINOXATEOCTISALATETITANIUM DIOXIDE\ZINC OXIDE:Thyroid disorder:0.25), (EPHEDRINE HYDROCHLORIDEGUAIFENESIN:Thyroid disorder:0.06), (ERTAPENEM SODIUM:Thyroid disorder:0.15), (ESTRADIOL BENZOATE:Thyroid disorder:0.18), (ESTRADIOL HEMIHYDRATE:Thyroid disorder:0.63), (ESTRADIOLLEVONORGESTREL:Thyroid disorder:0.41), (ESTROGENSMETHYLTESTOSTERONE:Thyroid disorder:0.2), (ETHINYL ESTRADIOLFERROUS FUMARATENORETHINDRONE:Thyroid disorder:0.08), (ETHINYL ESTRADIOLFERROUS FUMARATENORETHINDRONE ACETATE:Thyroid disorder:0.35), (FERRIC POLYSACCHARIDE COMPLEX:Thyroid disorder:0.4), (FERROUS SULFATEGLYCINE:Thyroid disorder:0.56), (FLUCLOXACILLIN:Thyroid disorder:0.02), (FLUOXETINE HYDROCHLORIDEOLANZAPINE:Thyroid disorder:0.35), (FROVATRIPTAN SUCCINATE:Thyroid disorder:1.2), (GABAPENTIN ENACARBIL:Thyroid disorder:0.13), (GIMERACILOTERACILTEGAFUR:Thyroid disorder:0.1), (GLIMEPIRIDEROSIGLITAZONE MALEATE:Thyroid disorder:0.34), (GLIPIZIDEMETFORMIN HYDROCHLORIDE:Thyroid disorder:1.08), (GLYCOPYRROLATEINDACATEROL MALEATE:Thyroid disorder:1.02), (GOLD:Thyroid disorder:0.41), (HALCINONIDE:Thyroid disorder:0.3), (HEPARIN CALCIUM:Thyroid disorder:0.17), (HERBALSMINERALSVITAMINS:Thyroid disorder:0.26), (HORSE CHESTNUT:Thyroid disorder:0.87), (HYDRAZINE SULFATE:Thyroid disorder:0.19), (HYDROCHLORIC ACID:Thyroid disorder:0.13), (HYDROXOCOBALAMIN:Thyroid disorder:0.1), (HYDROXYCITRIC ACID:Thyroid disorder:0.43), (ILOPROST:Thyroid disorder:1.45), (INFLIXIMAB-DYYB:Thyroid disorder:0.9), (INFLUENZA VIRUS VACCINE TRIVALENT TYPE A+B AFLURIA:Thyroid disorder:0.01), (INTERFERON:Thyroid disorder:1.16), (INTERFERON ALFA-2A:Thyroid disorder:1.12), (INTERFERON GAMMA-1B:Thyroid disorder:0.2), (IRINOTECAN:Thyroid disorder:0.19), (IRINOTECAN HYDROCHLORIDE:Thyroid disorder:0.82), (IRON SUCROSE:Thyroid disorder:0.9), (IRONPOLYSACCHARIDES:Thyroid disorder:0.42), (ISOPROPYL ALCOHOLLEUPROLIDE ACETATENORETHINDRONE ACETATE:Thyroid disorder:0.44), (IXAZOMIB:Thyroid disorder:1.01), (LABETALOL HYDROCHLORIDE:Thyroid disorder:1.31), (LACTOBACILLUS ACIDOPHILUS\LACTOBACILLUS DELBRUECKII SUBSP. BULGARICUS:Thyroid disorder:0.22), (LEUCOVORIN CALCIUM:Thyroid disorder:0.61), (LEVOBUNOLOL HYDROCHLORIDE:Thyroid disorder:0.31), (LEVOMEFOLIC ACIDMETHYLCOBALAMINTURMERIC:Thyroid disorder:0.46), (LORMETAZEPAM:Thyroid disorder:0.17), (LUTETIUM LU-177:Thyroid disorder:1.07), (MECLIZINE MONOHYDROCHLORIDENIACIN:Thyroid disorder:0.37), (MECLOZINE DIHYDROCHLORIDE:Thyroid disorder:0.05), (MENTHOLZINC OXIDE:Thyroid disorder:0.3), (MEPOLIZUMAB:Thyroid disorder:1.28), (METFORMIN HYDROCHLORIDESAXAGLIPTIN:Thyroid disorder:0.36), (METFORMINSITAGLIPTIN:Thyroid disorder:0.08), (METHYLPREDNISOLONE ACETATE:Thyroid disorder:2.96), (METRELEPTIN:Thyroid disorder:1.17), (MOXONIDINE:Thyroid disorder:1.08), (NADROPARIN CALCIUM:Thyroid disorder:1.05), (NAFTIFINE HYDROCHLORIDE:Thyroid disorder:0), (NALOXONE:Thyroid disorder:0.22), (NALOXONE HYDROCHLORIDEOXYCODONE HYDROCHLORIDE:Thyroid disorder:2.18), (NALOXONE HYDROCHLORIDETILIDINE HYDROCHLORIDE:Thyroid disorder:1.63), (NEOMYCIN:Thyroid disorder:0.54), (NICORANDIL:Thyroid disorder:0.62), (NOREPINEPHRINE BITARTRATE:Thyroid disorder:0.24), (OCTREOTIDE:Thyroid disorder:1.36), (OPIPRAMOL:Thyroid disorder:0.4), (OXALIPLATIN:Thyroid disorder:0.09), (PANITUMUMAB:Thyroid disorder:0.3), (PARSLEY:Thyroid disorder:0.03), (PEMETREXED:Thyroid disorder:0.64), (PENICILLIN G:Thyroid disorder:0.92), (PENICILLIN V:Thyroid disorder:0.62), (PIRBUTEROL ACETATE:Thyroid disorder:0.08), (POLYETHYLENE GLYCOL 3350POTASSIUM CHLORIDE\SODIUM BICARBONATE\SODIUM CHLORIDE\SODIUM SULFATE ANHYDROUS:Thyroid disorder:0.91), (POLYETHYLENE GLYCOL 4000:Thyroid disorder:0.56), (POLYVINYL ALCOHOL:Thyroid disorder:0.14), (POSACONAZOLE:Thyroid disorder:0.02), (PRAMOXINE HYDROCHLORIDE:Thyroid disorder:0.29), (PRAZOSIN HYDROCHLORIDE:Thyroid disorder:1.64), (PREDNISOLONE ACETATESULFACETAMIDE SODIUM:Thyroid disorder:0.33), (PREDNISOLONE SODIUM PHOSPHATE:Thyroid disorder:0.39), (PREDNISOLONEPREDNISOLONE ACETATEPREDNISOLONE SODIUM SUCCINATE:Thyroid disorder:0.6), (PROTRIPTYLINE HYDROCHLORIDE:Thyroid disorder:1.79), (PRUCALOPRIDE SUCCINATE:Thyroid disorder:0.29), (PYRAZINAMIDE:Thyroid disorder:0.8), (QUINACRINE:Thyroid disorder:0.83), (QUINIDINE GLUCONATE:Thyroid disorder:0.22), (RAMUCIRUMAB:Thyroid disorder:0.08), (SELEGILINE HYDROCHLORIDE:Thyroid disorder:0.47), (SODIUM BICARBONATE\SODIUM CHLORIDE:Thyroid disorder:0.03), (SODIUM PHOSPHATE, DIBASIC\SODIUM PHOSPHATE, MONOBASIC:Thyroid disorder:0.75), (SOFOSBUVIRVELPATASVIR:Thyroid disorder:0.92), (ST. JOHN'S WORT:Thyroid disorder:0.46), (SULFACETAMIDE SODIUMSULFUR:Thyroid disorder:0.57), (TAZAROTENE:Thyroid disorder:0.15), (TEDUGLUTIDE:Thyroid disorder:0.29), (TELOTRISTAT ETHYL:Thyroid disorder:1.15), (TERBUTALINE:Thyroid disorder:0.43), (TERBUTALINE SULFATE:Thyroid disorder:2.64), (TESAMORELIN:Thyroid disorder:0.98), (THEOPHYLLINE:Thyroid disorder:0.52), (TILIDINE:Thyroid disorder:0.18), (TIMOLOLTRAVOPROST:Thyroid disorder:0.54), (TIOPRONIN:Thyroid disorder:0.11), (TRAMETINIB DIMETHYL SULFOXIDE:Thyroid disorder:0.44), (TRANEXAMIC ACID:Thyroid disorder:0.22), (TREPROSTINIL DIOLAMINE:Thyroid disorder:0.28), (TRIHEXYPHENIDYL HYDROCHLORIDE:Thyroid disorder:0.03), (TROGLITAZONE:Thyroid disorder:0.02), (VALDECOXIB:Thyroid disorder:0.8), (VINCRISTINE SULFATE:Thyroid disorder:0.41), (VINORELBINE TARTRATE:Thyroid disorder:0.79), (VITAMIN A PALMITATE:Thyroid disorder:0.08), (VORAPAXAR SULFATE:Thyroid disorder:0.06), (ZILEUTON:Thyroid disorder:1.15), (AMPHETAMINE ASPARTATEAMPHETAMINE SULFATEDEXTROAMPHETAMINE SACCHARATEDEXTROAMPHETAMINE SULFATE:Thyroid function test abnormal:0.1), (BISOPROLOL FUMARATE:Thyroid function test abnormal:0.03), (CALCIUM CARBONATECHOLECALCIFEROL:Thyroid function test abnormal:0.15), (CERTOLIZUMAB PEGOL:Thyroid function test abnormal:0.03), (EVOLOCUMAB:Thyroid function test abnormal:0.03), (IBRUTINIB:Thyroid function test abnormal:0.05), (RANOLAZINE:Thyroid function test abnormal:0.09), (TRIAMCINOLONE ACETONIDE:Thyroid function test abnormal:0.16), (VARENICLINE TARTRATE:Thyroid function test abnormal:0.02), (ACETAMINOPHENBUTALBITALCAFFEINE:Thyroid hormone replacement therapy:0.03), (AMIODARONE HYDROCHLORIDE:Thyroid hormone replacement therapy:0.29), (AMOXICILLIN:Thyroid hormone replacement therapy:0.14), (ANASTROZOLE:Thyroid hormone replacement therapy:0.43), (ARMODAFINIL:Thyroid hormone replacement therapy:0.14), (BUSPIRONE HYDROCHLORIDE:Thyroid hormone replacement therapy:0.42), (CANDESARTAN CILEXETILHYDROCHLOROTHIAZIDE:Thyroid hormone replacement therapy:0.19), (CERTOLIZUMAB PEGOL:Thyroid hormone replacement therapy:0.58), (CLOTRIMAZOLE:Thyroid hormone replacement therapy:0.03), (COLCHICINE:Thyroid hormone replacement therapy:0.34), (DALFAMPRIDINE:Thyroid hormone replacement therapy:0.83), (DAPAGLIFLOZIN PROPANEDIOL:Thyroid hormone replacement therapy:0.37), (DASATINIB:Thyroid hormone replacement therapy:0.42), (DIPHENHYDRAMINE:Thyroid hormone replacement therapy:0.08), (ENOXAPARIN:Thyroid hormone replacement therapy:0.03), (ERYTHROPOIETIN:Thyroid hormone replacement therapy:0.01), (EVEROLIMUS:Thyroid hormone replacement therapy:0.59), (EVOLOCUMAB:Thyroid hormone replacement therapy:1.35), (HEPARIN SODIUM:Thyroid hormone replacement therapy:0.02), (INSULIN LISPRO:Thyroid hormone replacement therapy:1.35), (ISOSORBIDE:Thyroid hormone replacement therapy:0.24), (LIDOCAINE:Thyroid hormone replacement therapy:0.11), (LISDEXAMFETAMINE DIMESYLATE:Thyroid hormone replacement therapy:0.08), (METAXALONE:Thyroid hormone replacement therapy:0.41), (METRONIDAZOLE:Thyroid hormone replacement therapy:0.69), (NITROFURANTOIN:Thyroid hormone replacement therapy:0.04), (OLANZAPINE:Thyroid hormone replacement therapy:0.18), (OXCARBAZEPINE:Thyroid hormone replacement therapy:0.25), (OXYGEN:Thyroid hormone replacement therapy:0.31), (PANTOPRAZOLE MAGNESIUM:Thyroid hormone replacement therapy:0.26), (PAZOPANIB HYDROCHLORIDE:Thyroid hormone replacement therapy:0.05), (PEGFILGRASTIM:Thyroid hormone replacement therapy:0.11), (QUETIAPINE:Thyroid hormone replacement therapy:0.01), (SOTALOL:Thyroid hormone replacement therapy:0.28), (TAMSULOSIN:Thyroid hormone replacement therapy:1.21), (TELMISARTAN:Thyroid hormone replacement therapy:0.11), (TIZANIDINE HYDROCHLORIDE:Thyroid hormone replacement therapy:0.09), (TOCILIZUMAB:Thyroid hormone replacement therapy:0.03), (TOFACITINIB CITRATE:Thyroid hormone replacement therapy:0.3), (UMECLIDINIUM BROMIDEVILANTEROL TRIFENATATE:Thyroid hormone replacement therapy:0.23), (VARENICLINE TARTRATE:Thyroid hormone replacement therapy:0.32), (VITAMIN B:Thyroid hormone replacement therapy:0.36), (ENALAPRIL:Thyroid hormones decreased:0.11), (EVOLOCUMAB:Thyroid hormones decreased:0.33), (ACETAMINOPHENCODEINE PHOSPHATE:Thyroid therapy:0.1), (AMIODARONE:Thyroid therapy:0.09), (AMPHETAMINE ASPARTATEAMPHETAMINE SULFATEDEXTROAMPHETAMINE SACCHARATEDEXTROAMPHETAMINE SULFATE:Thyroid therapy:0.23), (ANASTROZOLE:Thyroid therapy:0.16), (CALCIUM CARBONATECHOLECALCIFEROL:Thyroid therapy:0.23), (EVEROLIMUS:Thyroid therapy:0.2), (EVOLOCUMAB:Thyroid therapy:0.67), (FERROUS SULFATEFERROUS SULFATE, DRIED:Thyroid therapy:0.26), (FINGOLIMOD HYDROCHLORIDE:Thyroid therapy:0.05), (IBRUTINIB:Thyroid therapy:0.34), (INSULIN HUMAN:Thyroid therapy:0.25), (ISOSORBIDE MONONITRATE:Thyroid therapy:0.4), (OLANZAPINE:Thyroid therapy:0.11), (OXCARBAZEPINE:Thyroid therapy:0.24), (OXYGEN:Thyroid therapy:0.07), (RAMIPRIL:Thyroid therapy:0.59), (RANOLAZINE:Thyroid therapy:0.52), (SODIUM OXYBATE:Thyroid therapy:0.09), (TIOTROPIUM BROMIDE MONOHYDRATE:Thyroid therapy:0.35), (ABIRATERONE ACETATE:Thyroidectomy:0.1), (ACARBOSE:Thyroidectomy:0.03), (ADO-TRASTUZUMAB EMTANSINE:Thyroidectomy:0.13), (ALCOHOL:Thyroidectomy:0.05), (AMANTADINE:Thyro idectomy:0.38), (AMLODIPINE BESYLATEOLMESARTAN MEDOXOMIL:Thyroidectomy:0.13), (AZITHROMYCIN DIHYDRATE:Thyroidectomy:0.08), (BUDESONIDEFORMOTEROL FUMARATE:Thyroidectomy:0.18), (BUPRENORPHINE HYDROCHLORIDENALOXONE HYDROCHLORIDE:Thyroidectomy:0.04), (CAPTOPRIL:Thyroidectomy:0.02), (CARFILZOMI B:Thyroidectomy:0.49), (CARVEDILOL PHOSPHATE:Thyroidectomy:0.51), (CEFTRIAXONE SODIUM:Thyroidectomy:0.13), (CEPHALEXIN:Thyroidectomy:1.39), (CLARITHROMYCIN:T hyroidectomy:0.05), (CLOTRIMAZOLE:Thyroidectomy:0.47), (CLOZAPINE:Thyroidectomy:0.32), (COD LIVER OIL:Thyroidectomy:0.13), (CODEINE:Thyroidectomy:0.2), (DABIGATRAN:Thyroidectomy:0.13), (DACLIZUMAB:Thyroidectomy:0.49), (DOCUSATE:Thyroidectomy:0.28), (DOCUSATE CALCIUM:Thyroidectomy:0.39), (DOXAZOSIN MESYLATE:Thyroidectomy:0.42), (DOXAZOSINDOXAZOSIN MESYLATE:Thyroidectomy:0.66), (DRONABINOL:Thyroidectomy:0.23), (DUTASTERIDE:T hyroidectomy:0.97), (ECONAZOLE:Thyroidectomy:0.11), (ELETRIPTAN HYDROBROMIDE:Thyroidectomy:0.92), (EMPAGLIFLOZINLINAGLIPTIN:Thyroidectomy: 0.17), (EPIRUBICIN:Thyroidectomy:0.08), (EPLERENONE:Thyroidectomy:0.22), (ERYTHRO MYCIN:Thyroidectomy:0.24), (ESTROGENS, CONJUGATEDMEDROXYPROGESTERONE ACETATE:Thyroidectomy:0.64), (ETORICOXIB:Thyroidectomy:0.43), (EVOLOCUMAB:Thyr oidectomy:1.36), (FENTANYL CITRATE:Thyroidectomy:0.03), (FERROUS FUMARATE:Thyroidectomy:0.21), (FLUTICASONE FUROATE:Thyroidectomy:0.24), (FLUTICASONE FUROATESALMETEROL XINAFOATE:Thyroidectomy:0.32), (FORMOTEROL FUMARATE DIHYDRATE\MOMETASONE FUROATE:Thyroidectomy:0.28), (GEMFIBROZIL:Thyroidectomy:0.07), (GOSERELIN ACETATE:Thyroidectomy:0.03), (HEPARIN CALCIUM:Thyroidectomy:0.01), (HYDROCHLOROTHIAZIDETELMISARTAN:Thyroidecto my:0.31), (HYOSCYAMINE:Thyroidectomy:0.29), (ILOPROST:Thyroidectomy:0.23), (IMIPRA MINE HYDROCHLORIDE:Thyroidectomy:0.4), (INDOMETHACIN:Thyroidectomy:0.31), (INFLUEN ZA VIRUS VACCINE:Thyroidectomy:0.15), (IRINOTECAN:Thyroidectomy:0.14), (ISOSORBIDE:Thyroid ectomy:0.81), (LABETALOLLABETALOL HYDROCHLORIDE:Thyroidectomy:0.5), (LINACLOTIDE:Thyroidectomy:0.12), (LOMITAPI DE MESYLATE:Thyroidectomy:0.1), (MECLIZINE HYDROCHLORIDE:Thyroidectomy:0.14), (MEGESTROL ACETATE:Thyroidectomy:0.05), (METHENAMINE HIPPURATE:Thyroidectomy:0.2), (METHYLPREDNISOLONE ACETATE:Thyroidectomy:0.49), (MYCOPHENOLATE MOFETILMYCOPHENOLATE MOFETIL HYDROCHLORIDE:Thyroidectomy:0.15), (NABUMETONE:Thyroidectomy:0.19), (NALOXO NE HYDROCHLORIDEOXYCODONE HYDROCHLORIDE:Thyroidectomy:0.81), (NORETHINDRONE:Thyroidectomy:0.09), (OXCA RBAZEPINE:Thyroidectomy:1.68), (PANTOPRAZOLE MAGNESIUM:Thyroidectomy:0.97), (PAROXETINEPAROXETINE HYDROCHLORIDE:Thyroidectomy:0.74), (PAZOPANIB HYDROCHLORIDE:Thyroidectomy:0.61), (PEGINTERFERON BETA-1A:Thyroidectomy:0.52), (PEMETREXED DISODIUM:Thyroidectomy:0.34), (PENICILLIN:Thyroidectomy:0.27), (PHENOBARBITAL:T hyroidectomy:0.57), (PLANTAGO SEED:Thyroidectomy:0.26), (PONATINIB:Thyroidectomy:0.51), (PREDNISOLONE ACETATE:Thyroidectomy:0.14), (PROCHLORPERAZINE MALEATE:Thyroidectomy:0.15), (PROMETHAZINE HYDROCHLORIDE:Thyroidectomy:0.27), (QUETIAPINE:Thyroidectomy:0.77), (RANOLAZI NE:Thyroidectomy:1.31), (REGORAFENIB:Thyroidectomy:0.14), (ROFLUMILAST:Thyroidect omy:0.42), (ROPINIROLE:Thyroidectomy:0.1), (SCOPOLAMINE:Thyroidectomy:0.27), (SENN A LEAFSENNOSIDESSENNOSIDES A AND B:Thyroidectomy:0.19), (SITAGLIPTIN:Thyroidectomy:0.01), (SODIUM CHLORIDE:Thyroidectomy:0.41), (TETRACYCLINE:Thyroidectomy:0.44), (THALIDOMIDE: Thyroidectomy:0.76), (TOLTERODINE TARTRATE:Thyroidectomy:0.5), (UMECLIDINIUM BROMIDEVILANTEROL TRIFENATATE:Thyroidectomy:0.59), (ZONISAMIDE:Thyroidectomy:0.31), (DALFAMPRIDI NE:Thyroiditis:0.06), (NAPROXEN:Thyroiditis:0.07), (PYRAZINAMIDE:Tinea infection:0.59), (ANASTROZOLE:Tinea pedis:0.22), (BUMETANIDE:Tinea pedis:0.04), (BUPROPION HYDROCHLORIDE:Tinea pedis:0.67), (CALCIUM CARBONATECHOLECALCIFEROL:Tinea pedis:0.47), (CLINDAMYCINCLINDAMYCIN PHOSPHATE:Tinea pedis:0.19), (CYCLOBENZAPRINE:Tinea pedis:0.12), (DALFAMPRIDINE:Tinea pedis:0.05), (DEXLANSOPRAZOLE:Tinea pedis:0.59), (DULOXETINE:Tinea pedis:0.05), (ENOXAPARIN:Tinea pedis:0.15), (EVOLOCUMAB:Tinea pedis:0.51), (FERROUS SULFATE:Tinea pedis:0.3), (FERROUS SULFATEFERROUS SULFATE, DRIED:Tinea pedis:0.46), (GLIPIZIDE:Tinea pedis:0.14), (IRBESARTAN:Tinea pedis:0.06), (NILOTINIB:Tinea pedis:0.02), (OXCARBAZEPINE:Tinea pedis:0.53), (OXYGEN:Tinea pedis:0.12), (POLYETHYLENE GLYCOLS:Tinea pedis:0.02), (POTASSIUM:Tinea pedis:0.54), (PRAMIPEXOLE DIHYDROCHLORIDE:Tinea pedis:0.01), (PRIMIDONE:Tinea pedis:0.06), (PROMETHAZINEPROMETHAZINE HYDROCHLORIDE:Tinea pedis:0.09), (SACUBITRILVALSARTAN:Tinea pedis:0.5), (TERIFLUNOMIDE:Tinea pedis:0.25), (TIZANIDINE HYDROCHLORIDE:Tinea pedis:0.07), (TOFACITINIB CITRATE:Tinea pedis:0.26), (TRIAMCINOLONE ACETONIDE:Tinea pedis:0.2), (AMIODARONE:Tinnitus:0.21), (BUDESONIDE:Tinnitus:0.46), (CALCIUM CARBONATECHOLECALCIFEROL:Tinnitus:0.61), (CERTOLIZUMAB PEGOL:Tinnitus:0.94), (CYCLOBENZAPRINE:Tinnitus:0.34), (DALFAMPRIDINE:Tinnitus:0.33), (DILTIAZEM:Tinnitus:0.36), (DIPHENHYDRAMINE:Tinnitus:0.11), (DIVALPROEX SODIUM:Tinnitus:0.09), (EPINEPHRINE:Tinnitus:0.13), (ESOMEPRAZOLE:Tinnitus:0.08), (E VOLOCUMAB:Tinnitus:0.91), (FERROUS SULFATEFERROUS SULFATE, DRIED:Tinnitus:0.83), (GLIPIZIDE:Tinnitus:0.16), (HYDROCHLOROTHIAZIDELISINOPRI L:Tinnitus:0.42), (INSULIN DETEMIR:Tinnitus:0.23), (ISOSORBIDE MONONITRATE:Tinnitus:0.01), (METAXALONE:Tinnitus:0.1), (METRONIDAZOLE:Tinnitu s:0.26), (NILOTINIB:Tinnitus:0.06), (NITROFURANTOIN:Tinnitus:0.01), (OXCARBAZEPINE: Tinnitus:0.35), (OXYGEN:Tinnitus:0.07), (POLYETHYLENE GLYCOLS:Tinnitus:0.13), (PRAVASTATIN SODIUM:Tinnitus:0.33), (PRIMIDONE:Tinnitus:0.01), (RABEPRAZOLE SODIUM:Tinnitus:0.22), (SACUBITRILVALSARTAN:Tinnitus:0.53), (TAMSULOSIN:Tinnitu s:0.69), (TERIFLUNOMIDE:Tinnitus:0.27), (UMECLIDINIUM BROMIDEVILANTEROL TRIFENATATE:Tinnitus:0.07), (VARENICLINE TARTRATE:Tinnitus:0.71), (BUSPIRONE HYDROCHLORIDE:Tobacco user:0.19), (CELECOXIB:Tobacco user:0.17), (DALFAMPRIDINE:Tobacco user:0.07), (DEXLANSOPRAZOLE:Tobacco user:0.41), (DIPHENHYDRAMINE:Tobacco user:0.02), (FERROUS SULFATEFERROUS SULFATE, DRIED:Tobacco user:0.06), (IBRUTINIB:Tobacco user:0.1), (INSULIN GLARGINE:Tobacco user:0.34), (LANSOPRAZOLE:Tobacco user:0.21), (LISDEXAMFETAMINE DIMESYLATE:Tobacco user:0), (MAGNESIUM:Tobacco user:0.05), (POTASSIUM:Tobacco user:0.2), (SACUBITRILVALSARTAN:Tobacco user:0.23), (UMECLIDINIUM BROMIDEVILANTEROL TRIFENATATE:Tobacco user:0.08), (VERAPAMIL HYDROCHLORIDE:Tobacco user:0.07), (WARFARIN:Tobacco user:0.76), (LACOSAMIDE:Tonic convulsion:0.16), (PYRAZINAMIDE:Tonsillitis:0.61), (DEXLANSOPRAZOLE:Tooth abscess:0.1), (ENALAPRIL:Tooth abscess:0.36), (EVOLOCUMAB:Tooth abscess:0.03), (FERROUS SULFATEFERROUS SULFATE, DRIED:Tooth abscess:0.01), (METHYLPREDNISOLONE SODIUM SUCCINATE:Tooth abscess:0.04), (SPIRONOLACTONE:Tooth abscess:0.28), (ANASTROZOLE:Tooth disorder:0.07), (BUSPIRONE HYDROCHLORIDE:Tooth disorder:0.08), (CERTOLIZUMAB PEGOL:Tooth disorder:0.14), (CHOLESTYRAMINE:Tooth disorder:0.12), (COLCHICINE:Tooth disorder:0.09), (DALFAMPRIDINE:Tooth disorder:0.31), (DIPHENHYDRAMINE:Tooth disorder:0.38), (ESCITALOPRAM OXALATE:Tooth disorder:0.48), (EVOLOCUMAB:Tooth disorder:0.39), (IBRUTINIB:Tooth disorder:0.08), (LOVASTATIN:Tooth disorder:0.41), (METHYLPREDNISOLONE SODIUM SUCCINATE:Tooth disorder:0.19), (NIFEDIPINE:Tooth disorder:0.05), (OXCARBAZEPINE:Tooth disorder:0.1), (PHENYTOIN:Tooth disorder:0.01), (POLYETHYLENE GLYCOLS:Tooth disorder:0.01), (PRAMIPEXOLE DIHYDROCHLORIDE:Tooth disorder:0.02), (TAMSULOSIN:Tooth disorder:0.3), (BUMETANIDE:Tooth extraction:0.05), (EVEROLIMUS:Tooth extraction:0.06), (CALCIUM CARBONATECHOLECALCIFEROL:Tooth infection:0.18), (CORTISONEHYDROCORTISONE:Tooth infection:0.12), (DOCUSATE SODIUM:Tooth infection:0.36), (ENALAPRIL:Tooth infection:0.64), (EVOLOCUMAB:Tooth infection:0.5), (IBRUTINIB:Tooth infection:0.47), (MELATONIN:Tooth infection:0.1), (SACUBITRILVALSARTAN:Tooth infection:0.22), (TERIFLUNOMIDE:Tooth infection:0.52), (TOPIRAMATE:Tooth infection:0.35), (VERAPAMIL HYDROCHLORIDE:Tooth infection:0.23), (AMIODARONE HYDROCHLORIDE:Toothache:0.2), (AMPHETAMINE ASPARTATEAMPHETAMINE SULFATEDEXTROAMPHETAMINE SACCHARATEDEXTROAMPHETAMINE SULFATE:Toothache:0.38), (ANASTROZOLE:Toothache:0.53), (ARMODAFINIL:Toothache:00.01), (CALCIUM CARBONATECHOLECALCIFEROL:Toothache:0.8), (CANDESARTAN:Toothache:0.09), (C HOLESTYRAMINE:Toothache:0.17), (CLOTRIMAZOLE:Toothache:0.04), (COLCHICINE:To othache:0.37), (CORTISONEHYDROCORTISONE:Toothache:0.08), (DILTIAZEM HYDROCHLORIDE:Toothache:1.03), (DOCUSATE SODIUM:Toothache:0.92), (ENOXAPARIN:Toothache:0.56), (EVOLOCUMAB:Toothache:0.7), (FINGOLIMOD HYDROCHLORIDE:Toothache:0.39), (FLECAINIDE:Toothache:0.08), (HYDROCHLOROTHI AZIDELISINOPRIL:Toothache:0.35), (IBRUTINIB:Toothache:0.73), (LOVASTATIN:Toothac he:0.55), (METAXALONE:Toothache:0.04), (METHYLPHENIDATE HYDROCHLORIDE:Toothache:0.1), (METHYLPREDNISOLONE ACETATE:Toothache:0.1), (OXYGEN:Toothache:0.19), (POLYETHYLENE GLYCOLS:Toothache:0.46), (PRAMIPEXOLE DIHYDROCHLORIDE:Toothache:0.21), (PROGESTERONE:Toothache:0.37), (PROMETHAZI NE HYDROCHLORIDE:Toothache:0.1), (RANOLAZINE:Toothache:0.36), (RISEDRONATE SODIUM:Toothache:0.18), (SACUBITRILVALSARTAN:Toothache:0.36), (SECUKINUMAB: Toothache:0.58), (SUMATRIPTANSUMATRIPTAN SUCCINATE:Toothache:0), (TOCILIZUMAB:Toothache:0.15), (TOFACITINIB CITRATE:Toothache:0.08), (UMECLIDINIUM BROMIDEVILANTEROL TRIFENATATE:Toothache:0.13), (VENLAFAXINE HYDROCHLORIDE:Toothache:1.04), (VITAMIN B:Toothache:0.08), (BUPRENORPHINE HYDROCHLORIDENALOXONE HYDROCHLORIDE:Torticollis:0.01), (DEXLANSOPRAZOLE:Torticollis:0.1), (DILTIAZEM HYDROCHLORIDE:Torticollis:0), (DIPHENHYDRAMINE:Torticollis:0.05), (EVOLOCUMAB: Torticollis:0.06), (HYDROXYZINE PAMOATE:Torticollis:0.1), (IRON:Torticollis:0.03), (OXCARBAZEPINE:Torticollis:0.45), (PA ROXETINE:Torticollis:0.07), (PROGESTERONE:Torticollis:0.08), (TERIFLUNOMIDE:Tortico Ills:0.69), (DEXLANSOPRAZOLE:Tourette's disorder:0.04), (VARENICLINE TARTRATE:Tourette's disorder:0.49), (BISOPROLOL FUMARATE:Toxicity to various agents:0.03), (BUPROPION HYDROCHLORIDE:Toxicity to various agents:0.21), (DILTIAZEM:Toxicity to various agents:0.06), (DULOXETINE HYDROCHLORIDE:Toxicity to various agents:0.23), (ENOXAPARIN:Toxicity to various agents:0.43), (FLUTICASONE PROPIONATE:Toxicity to various agents:0.08), (IBRUTINIB:Toxicity to various agents:0.76), (RISEDRONATE SODIUM:Toxicity to various agents:0), (SACUBITRILVALSARTAN:Toxicity to various agents:0.1), (TAMSULOSIN:Toxicity to various agents:0.02), (TOCILIZUMAB:Toxicity to various agents:0.19), (ATAZANAVIR:Toxoplasmosis:0.6), (ACETAMINOPHENCODEINE PHOSPHATE:Transfusion:0.27), (ALBUTEROL SULFATEIPRATROPIUM BROMIDE:Transfusion:0.17), (ALFACALCIDOL:Transfusion:0.11), (ANASTROZOLE:Transfu sion:0.25), (ATORVASTATIN CALCIUM:Transfusion:0.67), (BISACODYL OR DOCUSATE SODIUM:Transfusion:0.06), (CALCITRIOL:Transfusion:0.61), (CELECOXIB:Transfusion:0.26), (CHOLESTYRAMINE:Transfusion:0.34), (CLOZAPINE:Transfusion:0.05), (CYANOCOBALA MIN:Transfusion:0.22), (DASATINIB:Transfusion:0.28), (DEXLANSOPRAZOLE:Transfusion: 0.57), (ESZOPICLONE:Transfusion:0.34), (EVEROLIMUS:Transfusion:0.26), (FENOFIBRATE: Transfusion:0.05), (FERROUS SULFATE:Transfusion:0.57), (FERROUS SULFATEFERROUS SULFATE, DRIED:Transfusion:0.44), (GLIPIZIDE:Transfusion:0.14), (HYDROCHLOROTHIAZIDELISI NOPRIL:Transfusion:0.12), (IDELALISIB:Transfusion:0.22), (IMATINIB MESYLATE:Transfusion:0.23), (METHADONE HYDROCHLORIDE:Transfusion:0.02), (PREDNISOLONEPREDNISOLONE ACETATEPREDNISOLONE SODIUM SUCCINATE:Transfusion:0.15), (PROMETHAZINEPROMETHAZINE HYDROCHLORIDE:Transfusion:0.29), (RAMIPRIL:Transfusion:1.03), (SACUBITRILVALSA RTAN:Transfusion:0.31), (SULFAMETHOXAZOLE:Transfusion:0.39), (THALIDOMIDE:Tran sfusion:0.91), (TINZAPARIN SODIUM:Transfusion:0), (TOPIRAMATE:Transfusion:0.25), (VERAPAMIL HYDROCHLORIDE:Transfusion:0.33), (VINCRISTINE SULFATE:Transfusion:0.28), (ACARBOSE:Transient ischaemic attack:0.52), (ACETAMINOPHENBUTALBITALCAFFEINE:Transient ischaemic attack:0.84), (ACETAMINOPHENOXYCODONEOXYCODONE HYDROCHLORIDE:Transient ischaemic attack:0.18), (ACETAZOLAMIDE:Transient ischaemic attack:0.57), (ADO-TRASTUZUMAB EMTANSINE:Transient ischaemic attack:0.19), (ALCOHOL:Transient ischaemic attack:0.03), (AMANTADINE:Transient ischaemic attack:0.4), (AMISULPRIDE:Transient ischaemic attack:0.18), (AMLODIPINE BESYLATEHYDROCHLOROTHIAZIDEOLMESARTAN MEDOXOMIL:Transient ischaemic attack:0.29), (AMLODIPINEVALSARTAN:Transient ischaemic attack:0.12), (AMPHETAMINE ASPARTATEAMPHETAMINE SULFATEDEXTROAMPHETAMINE SACCHARATEDEXTROAMPHETAMINE SULFATE:Transient ischaemic attack:1.03), (AXITINIB:Transient ischaemic attack:0), (BENAZEPRIL HYDROCHLORIDE:Transient ischaemic attack:0.9), (BUDESONIDEFORMOTEROL FUMARATE:Transient ischaemic attack:0.39), (CANAGLIFLOZINMETFORMIN HYDROCHLORIDE:Transient ischaemic attack:0.11), (CARVEDILOL PHOSPHATE:Transient ischaemic attack:1.06), (CHONDROITIN SULFATE (BOVINE)GLUCOSAMINE HYDROCHLORIDE:Transient ischaemic attack:0.14), (CIMETIDINE:Transient ischaemic attack:0.15), (CLOTRIMAZOLE:Transient ischaemic attack:0.83), (CORTISONEHYDROCORTISONE:Transient ischaemic attack:0.79), (DANAZOL:Transient ischaemic attack:0.28), (DASATINIB:Transient ischaemic attack:1.69), (DEFLAZACORT:Transient ischaemic attack:0.55), (DEXAMETHASONE ACETATE:Transient ischaemic attack:0.03), (DIMENHYDRINATE:Transient ischaemic attack:0.14), (DOXEPIN HYDROCHLORIDE:Transient ischaemic attack:0.35), (DRONABINOL:Transient ischaemic attack:0.4), (DUTASTERIDETAMSULOSIN HYDROCHLORIDE:Transient ischaemic attack:0.04), (ECONAZOLE:Transient ischaemic attack:0.07), (EDOXABAN TOSYLATE:Transient ischaemic attack:0.95), (ELETRIPTAN HYDROBROMIDE:Transient ischaemic attack:0.67), (EMPAGLIFLOZINLINAGLIPTIN:Transient ischaemic attack:0.32), (EPLERENONE:Transient ischaemic attack:0.81), (ESTROGENS, CONJUGATEDMEDROXYPROGESTERONE ACETATE:Transient ischaemic attack:0.21), (ETHAMBUTOL HYDROCHLORIDE:Transient ischaemic attack:0.95), (FENTANYL CITRATE:Transient ischaemic attack:0.06), (FISH OIL\OMEGA-3 FATTY ACIDS:Transient ischaemic attack:0.03), (FLUTICASONE:Transient ischaemic attack:0.64), (FUSIDIC ACID:Transient ischaemic attack:0.01), (GLIMEPIRIDEROSIGLITAZONE MALEATE:Transient ischaemic attack:0), (HEPARIN CALCIUM:Transient ischaemic attack:0.12), (HERBALSTURMERIC:Transient ischaemic attack:0.22), (HYDROXYZINE PAMOATE:Transient ischaemic attack:1.04), (ILOPROST:Transient ischaemic attack:0.77), (IMATINIB MESYLATE:Transient ischaemic attack:1.05), (IMIPRAMINE HYDROCHLORIDE:Transient ischaemic attack:0.29), (INDAPAMIDEPERINDOPRIL:Transient ischaemic attack:1.28), (LIDOCAINE HYDROCHLORIDE:Transient ischaemic attack:0.55), (LIDOCAINEPRILOCAINE:Transient ischaemic attack:0.12), (LINACLOTIDE:Transient ischaemic attack:0.14), (LIOTHYRONINE SODIUM:Transient ischaemic attack:0.01), (LISDEXAMFETAMINE DIMESYLATE:Transient ischaemic attack:0.22), (LOMITAPIDE MESYLATE:Transient ischaemic attack:0.29), (MECLIZINEMECLIZINE HYDROCHLORIDE:Transient ischaemic attack:0.15), (MEGESTROL ACETATE:Transient ischaemic attack:0.31), (METAMIZOLE:Transient ischaemic attack:0.05), (METAXALONE:Transient ischaemic attack:1.01), (METFORMIN HYDROCHLORIDEROSIGLITAZONE MALEATE:Transient ischaemic attack:0.35), (METHENAMINE HIPPURATE:Transient ischaemic attack:0.3), (METHYLPREDNISOLONE ACETATE:Transient ischaemic attack:0.55), (MINOCYCLINEMINOCYCLINE HYDROCHLORIDE:Transient ischaemic attack:0.27), (MIRABEGRON:Transient ischaemic attack:0.18), (NABUMETONE:Transient ischaemic attack:0.2), (NALOXONE HYDROCHLORIDEOXYCODONE HYDROCHLORIDE:Transient ischaemic attack:0.96), (NATEGLINIDE:Transient ischaemic attack:0.21), (OLODATEROL HYDROCHLORIDETIOTROPIUM BROMIDE MONOHYDRATE:Transient ischaemic attack:0.67), (PARICALCITOL:Transient ischaemic attack:0.09), (PEGFILGRASTIM:Transient ischaemic attack:0.27), (PEGINTERFERON BETA-1A:Transient ischaemic attack:0.42), (PHENYTOIN SODIUM:Transient ischaemic attack:0.07), (PREDNISOLONEPREDNISOLONE ACETATEPREDNISOLONE SODIUM SUCCINATE:Transient ischaemic attack:0.17), (PROMETHAZINEPROMETHAZINE HYDROCHLORIDE:Transient ischaemic attack:0.75), (RADIUM RA-223 DICHLORIDE:Transient ischaemic attack:0.07), (SCOPOLAMINE:Transient ischaemic attack:0.55), (SECUKINUMAB:Transient ischaemic attack:0.74), (SULFAMETHOXAZOLE:Transient ischaemic attack:0.13), (TERBUTALINE SULFATE:Transient ischaemic attack:0.19), (TETRACYCLINE:Transient ischaemic attack:0.64), (THALIDOMIDE:Transient ischaemic attack:0.53), (THYROID, PORCINE:Transient ischaemic attack:0.23), (TIZANIDINE:Transient ischaemic attack:0.53), (TRASTUZUMAB:Transient ischaemic attack:0.11), (UMECLIDINIUM BROMIDEVILANTEROL TRIFENATATE:Transient ischaemic attack:1.05), (ZONISAMIDE:Transient ischaemic attack:0.06), (AMIODARONE HYDROCHLORIDE:Transitional cell carcinoma:0.43), (ANASTROZOLE:Transitional cell carcinoma:0.27), (BUPROPION HYDROCHLORIDE:Transitional cell carcinoma:0.08), (CALCIUM CARBONATECHOLECALCIFEROL:Transitional cell carcinoma:0.45), (DEXLANSOPRAZOLE:Transitional cell carcinoma:0.02), (DICLOFENAC:Transitional cell carcinoma:0.14), (ESCITALOPRAM OXALATE:Transitional cell carcinoma:0.26), (IBRUTINIB:Transitional cell carcinoma:0.38), (LENALIDOMIDE:Transitional cell carcinoma:0.33), (PYRAZINAMIDE:Transitional cell carcinoma:0.59), (TIOTROPIUM BROMIDE MONOHYDRATE:Transitional cell carcinoma:0.09), (VALSARTAN:Transitional cell carcinoma:0.27), (VERAPAMIL HYDROCHLORIDE:Transitional cell carcinoma:0.01), (ACETAMINOPHENCODEINE PHOSPHATE:Transplant:0.27), (AMIODARONE HYDROCHLORIDE:Transplant:0.48), (ANASTROZOLE:Transplant:0.21), (BENDAMUSTINE: Transplant:0.22), (BENDAMUSTINE HYDROCHLORIDE:Transplant:0.2), (BLINATUMOMAB:Transplant:0.38), (BRINCIDOFOVI R:Transplant:0.42), (CEPHALEXIN:Transplant:0.44), (CERTOLIZUMAB PEGOL:Transplant:0.19), (DICLOFENAC SODIUM:Transplant:0.3), (DILTIAZEM HYDROCHLORIDE:Transplant:0.52), (EVOLOCUMAB:Transplant:0.18), (GLIPIZIDE:Transpl ant:0.16), (GUSPERIMUS TRIHYDROCHLORIDE:Transplant:0.13), (HYDROCHLOROTHIAZIDELISINOPRIL:Transp lant:0.17), (IDELALISIB:Transplant:0.15), (IMIPENEM:Transplant:0.03), (INSULIN DETEMIR:Transplant:0.05), (LIDOCAINE:Transplant:0.17), (LOVASTATIN:Transplant:0.5), (MELOXICAM:Transplant:0.31), (NAPROXEN:Transplant:0.61), (NEBIVOLOL:Transplant:0.43), (OXYGEN:Transplant:0.31), (PARICALCITOL:Transplant:0.06), (PONATINIB:Transplant:0.19), (RABEPRAZOLE SODIUM:Transplant:0), (RANITIDINE HYDROCHLORIDE:Transplant:0.49), (RANOLAZINE:Transplant:0.39), (SACUBITRILVALS ARTAN:Transplant:0.46), (SECUKINUMAB:Transplant:0.15), (TELMISARTAN:Transplant:0.03), (TERIFLUNOMIDE:Transplant:0.03), (VERAPAMIL HYDROCHLORIDE:Transplant:0.53), (AMIODARONE:Transplant rejection:0.45), (BRINCIDOFOVIR:Transplant rejection:0.53), (CANDESARTAN:Transplant rejection:0.53), (CANDESARTAN CILEXETILHYDROCHLOROTHIAZIDE:Transplant rejection:0.08), (CEFEPIME HYDROCHLORIDE:Transplant rejection:0.25), (CEFTRIAXONE SODIUM:Transplant rejection:0.21), (CELECOXIB:Transplant rejection:0.4), (DASATINIB:Transplant rejection:0.09), (DICLOFENAC:Transplant rejection:0.09), (DIGOXIN:Transplant rejection:0.48), (ENOXAPARIN:Transplant rejection:1.15), (EPINEPHRINE:Transplant rejection:0.54), (ESZOPICLONE:Transplant rejection:0.22), (EVOLOCUMAB:Transplant rejection:0.34), (GLIMEPIRIDE:Transplant rejection:0.13), (IBRUTINIB:Transplant rejection:0.25), (IMIPENEM:Transplant rejection:0.06), (INDAPAMIDEPERINDOPRIL:Transplant rejection:0.33), (LEDIPASVIRSOFOSBUVIR:Transplant rejection:0.95), (LENALIDOMIDE:Transplant rejection:0.49), (LOVASTATIN:Transplant rejection:0.72), (MELOXICAM:Transplant rejection:0.26), (NAPROXEN:Transplant rejection:0.6), (NILOTINIB:Transplant rejection:0.29), (NITROFURANTOIN:Transplant rejection:0.22), (OMEPRAZOLE MAGNESIUM:Transplant rejection:0.42), (OXCARBAZEPINE:Transplant rejection:0.48), (OXYGEN:Transplant rejection:0.22), (PANTOPRAZOLE MAGNESIUM:Transplant rejection:0), (POLYETHYLENE GLYCOLS:Transplant rejection:0.25), (PROMETHAZINEPROMETHAZINE HYDROCHLORIDE:Transplant rejection:0.08), (RANOLAZINE:Transplant rejection:0.37), (TIMOLOL:Transplant rejection:0.08), (TOPIRAMATE:Transplant rejection:0.69), (TORSEMIDE:Transplant rejection:0.38), (TRAZODONE HYDROCHLORIDE:Transplant rejection:0.56), (ZONISAMIDE:Transplant rejection:0.15), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Tremor:0.12), (.ALPHA.1-PROTEINASE INHIBITOR HUMAN:Tremor:0.42), (ABOBOTULINUMTOXINA:Tremor:0.35), (ACETAMINOPHENOX YCODONEOXYCODONE HYDROCHLORIDE:Tremor:1.03), (AGALSIDASE BETA:Tremor:0.45), (ALBIGLUTIDE:Tremor:0.54), (ALENDRONIC ACID:Tremor:0.83), (ALISKIREN HEMIFUMARATE:Tremor:0.25), (AMLODIPINE BESYLATEVALSARTAN:Tremor:0.08), (ANTIHISTAMINES NOS:Tremor:0.23), (APOMORPHINE:Tremor:0.14), (APREPITANT:Tremor:0.17), (ATOMOX ETINE:Tremor:0.25), (BECLOMETHASONEFORMOTEROL:Tremor:0.07), (BETAMETHAS ONECALCIPOTRIENE:Tremor:0.1), (BUDESONIDEFORMOTEROL FUMARATE:Tremor:0.84), (BUPRENORPHINENALOXONE:Tremor:0.4), (BUTYLSCOPOL AMINE BROMIDE:Tremor:0.22), (CALCIUM CARBONATEVITAMIN D:Tremor:0.41), (CANAGLIFLOZINMETFORMIN HYDROCHLORIDE:Tremor:0.75), (CANAKINUMAB:Tremor:0.75), (CANDESARTAN CILEXETILHYDROCHLOROTHIAZIDE:Tremor:1.23), (CARVEDILOL PHOSPHATE:Tremor:1.65), (CILASTATIN SODIUMIMIPENEM:Tremor:0.05), (CLOBAZAM:Tremor:0.87), (CLOMIPRAMINE:Tremor: 0.16), (CLOMIPRAMINE HYDROCHLORIDE:Tremor:0.25), (CLOSTRIDIUM BUTYRICUM SPORES STRAIN M-55:Tremor:0.13), (CODEINE PHOSPHATE:Tremor:0.06), (DABIGATRAN:Tremor:0.21), (DACLIZUMAB:Tremor:1.16), (D ALTEPARIN SODIUM:Tremor:0.08), (DANAZOL:Tremor:0.67), (DESVENLAFAXINE:Tremor:0.05), (DEVI CE:Tremor:1.29), (DEXTROAMPHETAMINE SULFATE:Tremor:0.43), (DOXEPIN HYDROCHLORIDE:Tremor:0.78), (DOXYCYCLINE HYCLATE:Tremor:0.22), (DUTASTERIDETAMSULOSIN HYDROCHLORIDE:Tremor:0.17), (EDOXABAN TOSYLATE:Tremor:0.7), (EMPAGLIFLOZINLINAGLIPTIN:Tremor:0.92), (EXEMESTANE: Tremor:0.33), (FENTANYL CITRATE:Tremor:0.02), (FERROUS FUMARATE:Tremor:0.09), (FEXOFENADINEPSEUDOEPHEDRINE:Tremor:0.2), (FLUTIC ASONE FUROATESALMETEROL XINAFOATE:Tremor:1), (FORMOTEROL FUMARATE\MOMETASONE FUROATE:Tremor:0.59), (GARLIC:Tremor:0.21), (GLYCOPYRROLATEINDACATEROL MALEATE:Tremor:0.09), (GLYCOPYRRONIUMINDACATEROL:Tremor:0.82), (GUANFAC INE HYDROCHLORIDE:Tremor:1.15), (HERBALSTURMERIC:Tremor:0.65), (HYDROCODONE BITARTRATEIBUPROFEN:Tremor:0.17), (HYDROXYUREA:Tremor:0.62), (ILOPROST:Tre mor:0.42), (INDAPAMIDEPERINDOPRIL:Tremor:0.74), (ISOSORBIDE DINITRATE:Tremor:0.26), (IXAZOMIB:Tremor:0.04), (LACTASE:Tremor:0.13), (LACTOBAC ILLUS RHAMNOSUS:Tremor:0.23), (LEDIPASVIRSOFOSBUVIR:Tremor:1.06), (LERCANIDIPINE HYDROCHLORIDE:Tremor:0.46), (LIDOCAINE HYDROCHLORIDE:Tremor:1.2), (LOMITAPIDE MESYLATE:Tremor:0.58), (MEMANTINE:Tremor:0.11), (MEPOLIZUMAB:Tremor:0.42), (ME THENAMINE HIPPURATE:Tremor:1.4), (METHYLPREDNISOLONE ACETATE:Tremor:1.46), (MIDODRINE HYDROCHLORIDE:Tremor:0.28), (MILK THISTLE:Tremor:0.03), (MINOCYCLINE HYDROCHLORIDE:Tremor:0.21), (MOXIFLOXACIN:Tremor:0.3), (NALOXONE HYDROCHLORIDEOXYCODONE HYDROCHLORIDE:Tremor:1.04), (NEBIVOLOL:Tremor:0.65), (NIRAPARIB:Tremor:0), (NO RETHINDRONE:Tremor:0.1), (OMEPRAZOLE SODIUM:Tremor:0.1), (OXYBUTYNINOXYBUTYNIN CHLORIDE:Tremor:0.06), (OXYMORPHONE:Tremor:0.3), (PAMIDRONATE DISODIUM:Tremor:0.11), (PANTOPRAZOLE MAGNESIUM:Tremor:1.5), (PARATHYROID HORMONE:Tremor:0.17), (PATIROMER:Tremor:0.22), (PAZOPANIB HYDROCHLORIDE:Tremor:1.01), (PEGFILGRASTIM:Tremor:0.47), (PENICILLIN:Tremor:1.07), (PHYTONADIONE:Tremor:0.2), (PIROXICAM:Tremor:0.48), (PONATINIB:Tremor:0.99), (PREDNISOLONE ACETATE:Tremor:0.54), (PROTRIPTYLINE HYDROCHLORIDE:Tremor:0), (QUINAPRIL:Tremor:0.17), (RASAGILINE:Tremor:0.15), (RE TINOL:Tremor:0.08), (SALMETEROL XINAFOATE:Tremor:0.37), (SITAGLIPTIN:Tremor:0.53), (SODIUM FLUORIDE:Tremor:0.04), (SULINDAC:Tremor:0.07), (TESTOSTERONE UNDECANOATE:Tremor:0.18), (TETRACYCLINE:Tremor:1.05), (TOLCAPONE:Tremor:0.05), (TOLTERODINE:Tremor:0.35), (UMECLIDINIUM BROMIDE:Tremor:0.4), (VANCOMYCIN HYDROCHLORIDE:Tremor:0.18), (VITAMIN A:Tremor:0.73), (ZILEUTON:Tremor:0.16), (ZINC:Tremor:0.84), (ZUCLOPENTHIXOL DECANOATE:Tremor:0.66), (ACENOCOUMAROL:Trigeminal neuralgia:0.05), (ACETAMINOPHENOXYCODONEOXYCODONE HYDROCHLORIDE:Trigeminal neuralgia:0.05), (ACETAZOLAMIDE:Trigeminal neuralgia:0.42), (ALCOHOL:Trigeminal neuralgia:0.18), (AMIODARONE:Trigeminal neuralgia:1.31), (AZITHROMYCIN DIHYDRATE:Trigeminal neuralgia:0.03), (BISACODYL OR DOCUSATE SODIUM:Trigeminal neuralgia:0.61), (BUMETANIDE:Trigeminal neuralgia:0.5), (CARFILZOMIB:Trigeminal neuralgia:0.33), (CARVEDILOL PHOSPHATE:Trigeminal neuralgia:0.53), (CILOSTAZOL:Trigeminal neuralgia:0.06), (CIMETIDINE:Trigeminal neuralgia:0.31), (COLCHICINE:Trigeminal neuralgia:0.55), (DASATINIB:Trigeminal neuralgia:0.77), (DEFLAZACORT:Trigeminal neuralgia:0.18), (DESVENLAFAXINE SUCCINATE:Trigeminal neuralgia:0.23), (DOFETILIDE:Trigeminal neuralgia:0.36), (EDOXABAN TOSYLATE:Trigeminal neuralgia:0.21), (ELETRIPTAN HYDROBROMIDE:Trigeminal neuralgia:0.83), (EPINEPHRINE:Trigeminal neuralgia:0.19), (EPLERENONE:Trigeminal neuralgia:0.07), (ETORICOXIB:Trigeminal neuralgia:0.38), (EVEROLIMUS:Trigeminal neuralgia:0.79), (EVOLOCUMAB:Trigeminal neuralgia:0.94), (EZETIMIBESIMVASTATIN:Trigeminal neuralgia:0.56), (FENOFIBRIC ACID:Trigeminal neuralgia:0.02), (FLUDROCORTISONE ACETATE:Trigeminal neuralgia:0.28), (FLUOXETINEFLUOXETINE HYDROCHLORIDE:Trigeminal neuralgia:0.69), (FORMOTEROL FUMARATE:Trigeminal neuralgia:0.03), (GLYCOPYRRONIUMINDACATEROL:Trigeminal neuralgia:0.06), (HALOPERIDOL:Trigeminal neuralgia:0.19), (HEPARIN SODIUM:Trigeminal neuralgia:0.52), (HYDROCHLOROTHIAZIDELOSARTAN POTASSIUM:Trigeminal neuralgia:0.44), (HYDROMORPHONE:Trigeminal neuralgia:0.35), (HYDROXYZINE PAMOATE:Trigeminal neuralgia:1.09), (HYOSCYAMINE:Trigeminal neuralgia:0.42), (IMATINIB MESYLATE:Trigeminal neuralgia:0.18), (INFLUENZA VIRUS VACCINE:Trigeminal neuralgia:0.05), (INSULIN HUMAN:Trigeminal neuralgia:0.43), (LABETALOLLABETALOL HYDROCHLORIDE:Trigeminal neuralgia:0.66), (LEDIPASVIRSOFOSBUVIR:Trigeminal neuralgia:0.2), (LERCANIDIPINE HYDROCHLORIDE:Trigeminal neuralgia:0.64), (LINAGLIPTIN:Trigeminal neuralgia:0.59), (LISDEXAMFETAMINE DIMESYLATE:Trigeminal neuralgia:0.46), (MOXIFLOXACIN HYDROCHLORIDE:Trigeminal neuralgia:0.47), (NABUMETONE:Trigeminal neuralgia:0.22), (PANTOPRAZOLE MAGNESIUM:Trigeminal neuralgia:0.76), (PENICILLIN:Trigeminal neuralgia:0.29), (PENTOSAN POLYSULFATE SODIUM:Trigeminal neuralgia:0.03), (POLYETHYLENE GLYCOLS:Trigeminal neuralgia:0.79), (PRIMIDONE:Trigeminal neuralgia:1.01), (PYRIDOXINE:Trigeminal neuralgia:0.14), (RABEPRAZOLE:Trigeminal neuralgia:0.52), (SACUBITRILVALSARTAN:Trigeminal neuralgia:1.28), (SECUKINUMAB:Trigeminal neuralgia:0.53), (TETRACYCLINE:Trigeminal neuralgia:0.6), (THALIDOMIDE:Trigeminal neuralgia:0.06), (TIMOLOL:Trigeminal neuralgia:0.71), (TOCILIZUMAB:Trigeminal neuralgia:0.51), (TRIAMTERENE:Trigeminal neuralgia:0.68), (VILAZODONE HYDROCHLORIDE:Trigeminal neuralgia:0.39), (ZOLEDRONIC ACID:Trigeminal neuralgia:0.45), (CALCIUM:Triple negative breast cancer:0.21), (DILTIAZEM HYDROCHLORIDE:Triple negative breast cancer:0.01), (FOLIC ACID:Triple negative breast cancer:0.33), (TOPIRAMATE:Triple negative breast cancer:0.1), (UNSPECIFIED INGREDIENT:Triple negative breast cancer:0.12), (ACENOCOUMAROL:Tuberculosis:0.02), (AMISULPRIDE:Tuberculosis:0.03), (A NASTROZOLE:Tuberculosis:0.28), (BELATACEPT:Tuberculosis:0.09), (BUPRENORPHINE HYDROCHLORIDE:Tuberculosis:0.06), (BUPROPION HYDROCHLORIDE:Tuberculosis:0.88), (CALCIUM ACETATE:Tuberculosis:0.07), (CANAKINUMAB:Tuberculosis:0.27), (CANDESARTAN CILEXETILHYDROCHLOROTHIAZIDE:Tuberculosis:0.54), (COBICISTATELVITEGRAVI REMTRICITABINETENOFOVIR:Tuberculosis:0.14), (DALFAMPRIDINE:Tuberculosis:0.2), (DASATINIB:Tuberculosis:0.57), (DIVALPROEX SODIUM:Tuberculosis:1.38), (DOCUSATE SODIUM:Tuberculosis:0.9), (DOXAZOSINDOXAZOSIN MESYLATE:Tuberculosis:0.26), (ECONAZOLE:Tuberculosis:0.34), (EMTRICITABINERILPI VIRINE HYDROCHLORIDETENOFOVIR DISOPROXIL FUMARATE:Tuberculosis:0.11), (ESZOPICLONE:Tuberculosis:0.66), (ETORICOXIB:Tubercul osis:0.54), (EVOLOCUMAB:Tuberculosis:0.41), (FISH OIL:Tuberculosis:0.92), (FLUTICASONE:Tuberculosis:0.21), (FLUTICASONE FUROATEVILANTEROL TRIFENATATE:Tuberculosis:0.12), (FORMOTEROL:Tuberculosis:0), (FUSIDIC ACID:Tuberculosis:0.06), (GLYCOPYRRONIUMINDACATEROL:Tuberculosis:0.03), (HYDR OCHLOROTHIAZIDELISINOPRIL:Tuberculosis:0.11), (HYDROCHLOROTHIAZIDEVALS ARTAN:Tuberculosis:0.29), (HYDROMORPHONE:Tuberculosis:0.49), (HYDROXYCHLORO QUINE DIPHOSPHATE:Tuberculosis:0.3), (HYDROXYUREA:Tuberculosis:0.67), (IBRUTINIB:Tuber culosis:1.46), (ILOPROST:Tuberculosis:0.24), (INFLIXIMAB-DYYB:Tuberculosis:0.32), (INSULIN LISPRO:Tuberculosis:0.05), (INVESTIGATIONAL PRODUCT:Tuberculosis:0.65), (LINAGLIPTIN:Tuberculosis:0.14), (METHYLPREDNISOLONE ACETATE:Tuberculosis:0.35), (MICAFUNGIN:Tuberculosis:0.85), (NELFINAVIR:Tuberculosi s:0.28), (OMEPRAZOLE MAGNESIUM:Tuberculosis:0.34), (OSELTAMIVIR:Tuberculosis:0.27), (PANTOPRAZOLE MAGNESIUM:Tuberculosis:0.54), (PEGINTERFERON ALFA-2A OR PEGINTERFERON ALFA-2B OR PEGINTERFERON BETA-1A OR PEGINTERFERON LAMBDA-1A:Tuberculosis:1.1), (PONATINIB:Tuberculosis:0.58), (PRAMIPEXOLE DIHYDROCHLORIDE:Tuberculosis:0), (PRAVASTATIN:Tuberculosis:0.72), (PREDNISOLO NE SODIUM PHOSPHATE:Tuberculosis:0.55), (PRIMIDONE:Tuberculosis:0.32), (PROBIOTICS NOS:Tuberculosis:0.1), (PROCHLORPERAZINE MALEATE:Tuberculosis:0.04), (PROGESTERONE:Tuberculosis:0.17), (PROMETHAZINE HYDROCHLORIDE:Tuberculosis:0.35), (SACUBITRILVALSARTAN:Tuberculosis:0.52), (SO TALOL:Tuberculosis:0.69), (TEMAZEPAM:Tuberculosis:0.18), (TETRACYCLINE:Tuberculosi s:0.37), (TIOTROPIUM:Tuberculosis:0.15), (TIZANIDINE HYDROCHLORIDE:Tuberculosis:0.17), (TOPIRAMATE:Tuberculosis:1.52), (ETHAMBUTOL HYDROCHLORIDE:Tuberculosis of intrathoracic lymph nodes:0.7), (PYRAZINAMIDE:Tuberculosis ureter:0.52), (BRIVARACETAM:Tuberous sclerosis:0.13), (ANASTROZOLE:Tumour lysis syndrome:0.24), (BUPRENORPHINE HYDROCHLORIDE:Tumour lysis syndrome:0.29), (CALCITRIOL:Tumour lysis syndrome:0.48), (CANDESARTAN CILEXETILHYDROCHLOROTHIAZIDE:Tumour lysis syndrome:0.57), (CEFOTAXIME SODIUM:Tumour lysis syndrome:0.25), (CERTOLIZUMAB PEGOL:Tumour lysis syndrome:0.8), (CHOLESTYRAMINE:Tumour lysis syndrome:0.63), (CLINDAMYCIN:Tumour lysis syndrome:0.14), (CLINDAMYCINCLINDAMYCIN PHOSPHATE:Tumour lysis syndrome:1.66), (CLOZAPINE:Tumour lysis syndrome:0.36), (CYCLOBENZAPRINE:Tumour lysis syndrome:0.2), (DEXLANSOPRAZOLE:Tumour lysis syndrome:0.57), (DICLOFENAC SODIUM:Tumour lysis syndrome:0.34), (DUTASTERIDE:Tumour lysis syndrome:0.27), (ELOTUZUMAB:Tumour lysis syndrome:0.04), (ETOPOSIDE PHOSPHATE:Tumour lysis syndrome:0.02), (GLYBURIDE:Tumour lysis syndrome:0.03), (HYDROCHLOROTHIAZIDELISINOPRIL:Tumour lysis syndrome:0.51), (INDAPAMIDEPERINDOPRIL:Tumour lysis syndrome:0.23), (IPRATROPIUM:Tumour lysis syndrome:0.22), (IRON:Tumour lysis syndrome:0.68), (ISOSORBIDE:Tumour lysis syndrome:0.09), (ITRACONAZOLE:Tumour lysis syndrome:0.26), (IXAZOMIB:Tumour lysis syndrome:0.01), (LABETALOLLABETALOL HYDROCHLORIDE:Tumour lysis syndrome:0.22), (LERCANIDIPINE HYDROCHLORIDE:Tumour lysis syndrome:0.52), (LEVALBUTEROL HYDROCHLORIDE:Tumour lysis syndrome:0.07), (LINAGLIPTIN:Tumour lysis syndrome:0.16), (MELOXICAM:Tumour lysis syndrome:0.47), (METHADONE HYDROCHLORIDE:Tumour lysis syndrome:0.24), (METHIMAZOLE:Tumour lysis syndrome:0.03), (MOXIFLOXACIN HYDROCHLORIDE:Tumour lysis syndrome:0.43), (NEBIVOLOL HYDROCHLORIDE:Tumour lysis syndrome:0.03), (OSELTAMIVIR:Tumour lysis syndrome:0.18), (OXCARBAZEPINE:Tumour lysis syndrome:0.15), (PAROXETINEPAROXETINE HYDROCHLORIDE:Tumour lysis syndrome:0.06), (PEMETREXED DISODIUM:Tumour lysis syndrome:0.06), (QUETIAPINE:Tumour lysis syndrome:0.28), (SACUBITRILVALSARTAN:Tumour lysis syndrome:0.59), (TIGECYCLINE:Tumour lysis syndrome:0.07), (TIOTROPIUM:Tumour lysis syndrome:0.13), (TOPIRAMATE:Tumour lysis syndrome:0.75), (VALGANCICLOVIR HYDROCHLORIDE:Tumour lysis syndrome:0.3), (ADO-TRASTUZUMAB EMTANSINE:Tumour pain:0.38), (AMIODARONE:Tumour pain:0.27), (AMIODARONE HYDROCHLORIDE:Tumour pain:0.16), (ANASTROZOLE:Tumour pain:0.87), (BUMETANIDE:Tumour pain:0.14), (BUPROPION HYDROCHLORIDE:Tumour pain:0.3), (CANDESARTAN:Tumour pain:0.72), (CANDESARTAN CILEXETILHYDROCHLOROTHIAZIDE:Tumour pain:0.27), (CEPHALEXIN:Tumour pain:0.24), (CERTOLIZUMAB PEGOL:Tumour pain:0.61), (CLINDAMYCINCLINDAMYCIN PHOSPHATE:Tumour pain:0.08), (CLOZAPINE:Tumour pain:0.07), (CORTISONEHYDROCORTISONE:Tumour pain:0.19), (DALTEPARIN SODIUM:Tumour pain:0.18), (DASATINIB:Tumour pain:0.46), (DIGOXIN:Tumour pain:0.47), (DILTIAZEM:Tumour pain:0.14), (EPIRUBICIN:Tumour pain:0.39), (FULVESTRANT:Tumour pain:0.35), (IMATINIB MESYLATE:Tumour pain:0.15), (INDAPAMIDE:Tumour pain:0.03), (INDAPAMIDEPERINDOPRIL:Tumour pain:0.11), (IRINOTECAN HYDROCHLORIDE:Tumour pain:0.75), (IRON:Tumour pain:0.26), (ISOSORBIDE MONONITRATE:Tumour pain:0.02), (LEVOLEUCOVORIN CALCIUM:Tumour pain:0.39), (LOVASTATIN:Tumour pain:0.22), (MELOXICAM:Tumour pain:0.2), (METOPROLOL TARTRATE:Tumour pain:0.21), (NAPROXEN:Tumour pain:0.92), (NILOTINIB:Tumour pain:0.73), (SACUBITRILVALSARTAN:Tumour pain:0.19), (SOTALOL:Tumour pain:0.18), (TELMISARTAN:Tumour pain:0.45), (THALIDOMIDE:Tumour pain:0.41), (WARFARIN:Tumour pain:0.44), (ACETAMINOPHEN\CAFFEINEOPIUM:Type 1 diabetes mellitus:0.26), (ACETAMINOPHENHYDROCODONE:Type 1 diabetes mellitus:0.8), (ACETAMINOPHENPROPOXYPHENE HYDROCHLORIDE:Type 1 diabetes mellitus:0.02), (AGALSIDASE BETA:Type 1 diabetes mellitus:0.73), (ALISKIREN HEMIFUMARATE:Type 1 diabetes mellitus:0.55), (ALUMINUM HYDROXIDEDIMETHICONEMAGNESIUM HYDROXIDE:Type 1 diabetes mellitus:0.48), (AMANTADINE:Type 1 diabetes mellitus:0.32), (AMIKACIN:Type 1 diabetes mellitus:0.09), (AMINO ACIDSDEXTROSEELECTROLYTES NOSSOYBEAN OILVITAMINS:Type 1 diabetes mellitus:0.08), (AMISULPRIDE:Type 1 diabetes mellitus:0.65), (AMLODIPINE BESYLATEHYDROCHLOROTHIAZIDEOLMESARTAN MEDOXOMIL:Type 1 diabetes mellitus:0.41), (AMLODIPINE BESYLATEHYDROCHLOROTHIAZIDEVALSARTAN:Type 1 diabetes mellitus:0.79), (AMLODIPINEVALSARTAN:Type 1 diabetes mellitus:0.79), (AMMONIUM LACTATE:Type 1 diabetes mellitus:0.32), (ANAGRELIDE HYDROCHLORIDE:Type 1 diabetes mellitus:0.01), (APREPITANT:Type 1 diabetes mellitus:0.26), (ARIPIPRAZOLE LAUROXIL:Type 1 diabetes mellitus:0.08), (ATENOLOLCHLORTHALIDONE:Type 1 diabetes mellitus:0.06), (ATOVAQUONE:Type 1 diabetes mellitus:0.24), (AZELASTINE:Type 1 diabetes mellitus:0.03), (AZILSARTAN KAMEDOXOMIL:Type 1 diabetes mellitus:0.84), (BELATACEPT:Type 1 diabetes mellitus:0), (BENZTROPINE:Type 1 diabetes mellitus:0.14), (BETAMETHASONE DIPROPIONATECALCIPOTRIENECALCIPOTRIENE HYDRATE:Type 1 diabetes mellitus:0.48), (BETHANECHOL CHLORIDE:Type 1 diabetes mellitus:0.02), (BIFIDOBACTERIUM LONGUM SUBSP. INFANTIS:Type 1 diabetes mellitus:0.3), (BOTULINUM TOXIN TYPE A:Type 1 diabetes mellitus:0.09), (BUDESONIDEFORMOTEROL FUMARATE:Type 1 diabetes mellitus:0.83), (BUPRENORPHINE HYDROCHLORIDE:Type 1 diabetes mellitus:1.02), (BUPRENORPHINE HYDROCHLORIDENALOXONE HYDROCHLORIDE:Type 1 diabetes mellitus:0.84), (BUTYLSCOPOLAMINE BROMIDE:Type 1 diabetes mellitus:0.34), (CALCIUM CHLORIDEDEXTROSE MONOHYDRATEMAGNESIUM CHLORIDE\SODIUM CHLORIDE\SODIUM LACTATE:Type 1 diabetes mellitus:0.38), (CALCIUM\MAGNESIUM:Type 1 diabetes mellitus:0.18), (CANDESARTAN CILEXETILHYDROCHLOROTHIAZIDE:Type 1 diabetes mellitus:2.17), (CARFILZOMIB:Type 1 diabetes mellitus:1.02), (CARVEDILOL PHOSPHATE:Type 1 diabetes mellitus:1.74), (CEFEPIME HYDROCHLORIDE:Type 1 diabetes mellitus:0.16), (CEFOTAXIME SODIUM:Type 1 diabetes mellitus:0.38), (CEFTAZIDIME:Type 1 diabetes mellitus:0.15), (CEFUROXIME AXETIL:Type 1 diabetes mellitus:0.1), (CETIRIZINE HYDROCHLORIDEPSEUDOEPHEDRINE HYDROCHLORIDE:Type 1 diabetes mellitus:0.41), (CHLORDIAZEPOXIDE HYDROCHLORIDECLIDINIUM BROMIDE:Type 1 diabetes mellitus:0.03), (CHONDROITIN SULFATE (BOVINE)GLUCOSAMINE HYDROCHLORIDE:Type 1 diabetes mellitus:0.47), (CLOBETASOL:Type 1 diabetes mellitus:0.77), (CRIZOTINIB:Type 1 diabetes mellitus:0.51), (CYPROHEPTADINE:Type 1 diabetes mellitus:0.38), (DABIGATRAN:Type 1 diabetes mellitus:1.56), (DACLATASVIR DIHYDROCHLORIDE:Type 1 diabetes mellitus:0.01), (DANAZOL:Type 1 diabetes mellitus:0.57), (DASATINIB:Type 1 diabetes mellitus:1.8), (DEXAMETHASONE ACETATE:Type 1 diabetes mellitus:0.03), (DEXTROAMPHETAMINE SULFATE:Type 1 diabetes mellitus:0.04), (DIGITOXIN:Type 1 diabetes mellitus:0.59), (DIPYRONE:Type 1 diabetes mellitus:0.17), (DOCUSATE CALCIUM:Type 1 diabetes mellitus:1.1), (DOXEPIN HYDROCHLORIDE:Type 1 diabetes mellitus:0.62), (DUTASTERIDETAMSULOSIN HYDROCHLORIDE:Type 1 diabetes mellitus:0.35), (EDOXABAN TOSYLATE:Type 1 diabetes mellitus:1.35), (ELBASVIRGRAZOPREVIR:Type 1 diabetes mellitus:0.23), (EPIRUBICIN:Type 1 diabetes mellitus:0.05), (ESTRADIOLNORETHINDRONE ACETATE:Type 1 diabetes mellitus:0.25), (ESTRONESUS SCROFA OVARY:Type 1 diabetes mellitus:0.25), (ETHAMBUTOL HYDROCHLORIDE:Type 1 diabetes mellitus:0.96), (ETORICOXIB:Type 1 diabetes mellitus:1.04), (FENOTEROL HYDROBROMIDE:Type 1 diabetes mellitus:0.06), (FENOTEROL HYDROBROMIDEIPRATROPIUM BROMIDE:Type 1 diabetes mellitus:0.09), (FLUOCINONIDE:Type 1 diabetes mellitus:0.52), (FLUTICASONE FUROATEVILANTEROL TRIFENATATE:Type 1 diabetes mellitus:1.03), (FORMOTEROL FUMARATE:Type 1 diabetes mellitus:0.96), (FORMOTEROL FUMARATE DIHYDRATE\MOMETASONE FUROATE:Type 1 diabetes mellitus:0.95), (FORMOTEROL FUMARATE\MOMETASONE FUROATE:Type 1 diabetes mellitus:0.47), (FUSIDIC ACID:Type 1 diabetes mellitus:0.61), (GLYCOPYRROLATEINDACATEROL MALEATE:Type 1 diabetes mellitus:0.11), (GLYCOPYRRONIUM:Type 1 diabetes mellitus:0.18), (GLYCOPYRRONIUMINDACATEROL:Type 1 diabetes mellitus:1.02), (GUANFACINE:Type 1 diabetes mellitus:0.36), (GUANFACINE HYDROCHLORIDE:Type 1 diabetes mellitus:0.24), (HERBALSSAW PALMETTO:Type 1 diabetes mellitus:0.1), (HYALURONATE SODIUM:Type 1 diabetes mellitus:0.18), (HYDROCODONE BITARTRATEIBUPROFEN:Type 1 diabetes mellitus:0.04), (HYDROCORTISONE SODIUM SUCCINATE:Type 1 diabetes mellitus:0.12), (HYDROXYUREA:Type 1 diabetes mellitus:0.83), (HYOSCYAMINE:Type 1 diabetes mellitus:0.56), (IDELALISIB:Type 1 diabetes mellitus:0.25), (ILOPROST:Type 1 diabetes mellitus:0.53), (IMATINIB MESYLATE:Type 1 diabetes mellitus:1.5), (INDACATEROL:Type 1 diabetes mellitus:0.34), (INTERFERON ALFA-2A:Type 1 diabetes mellitus:0.15), (INTERFERON ALFA-2B:Type 1 diabetes mellitus:0.06), (IPRATROPIUM:Type 1 diabetes mellitus:1.43), (IXAZOMIB:Type 1 diabetes mellitus:0.22), (LANREOTIDE ACETATE:Type 1 diabetes mellitus:1.18), (LUBIPROSTONE:Type 1 diabetes mellitus:0.06), (MAGNESIUM ASPARTATE HYDROCHLORIDE:Type 1 diabetes mellitus:0), (MEPERIDINE:Type 1 diabetes mellitus:0.04), (METAXALONE:Type 1 diabetes mellitus:1.19), (METHENAMINE HIPPURATE:Type 1 diabetes mellitus:0.99), (METHYLPREDNISOLONE ACETATE:Type 1 diabetes mellitus:1.22), (MILK THISTLE:Type 1 diabetes mellitus:0.01), (MOEXIPRIL HYDROCHLORIDE:Type 1 diabetes mellitus:0.18), (NADROPARIN CALCIUM:Type 1 diabetes mellitus:0.63), (NALOXONE:Type 1 diabetes mellitus:0.01), (NALOXONE HYDROCHLORIDETILIDINE HYDROCHLORIDE:Type 1 diabetes mellitus:0.42), (NATEGLINIDE:Type 1 diabetes mellitus:1.2), (NIRAPARIB:Type 1 diabetes mellitus:0.19), (NOREPINEPHRINE BITARTRATE:Type 1 diabetes mellitus:0.01), (OLODATEROL HYDROCHLORIDETIOTROPIUM BROMIDE MONOHYDRATE:Type 1 diabetes mellitus:0.87), (ORPHENADRINE:Type 1 diabetes mellitus:0.08), (OSELTAMIVIR:Type 1 diabetes mellitus:0.08), (PAMIDRONATE DISODIUM:Type 1 diabetes mellitus:0.15), (PANTOPRAZOLE MAGNESIUM:Type 1 diabetes mellitus:1.59), (PARATHYROID HORMONE:Type 1 diabetes mellitus:0.12), (PARICALCITOL:Type 1 diabetes mellitus:1.12), (PEMETREXED:Type 1 diabetes mellitus:0.06), (PILOCARPINE:Type 1 diabetes mellitus:0.19), (POLYETHYLENE GLYCOL 4000:Type 1 diabetes mellitus:0.26), (PONATINIB:Type 1 diabetes mellitus:1.27), (POSACONAZOLE:Type 1 diabetes mellitus:0.29), (PRAZOSIN HYDROCHLORIDE:Type 1 diabetes mellitus:0.56), (PREDNISOLONE SODIUM PHOSPHATE:Type 1 diabetes mellitus:0.03), (PRISTINAMYCIN:Type 1 diabetes mellitus:0.07), (RIVASTIGMINE:Type 1 diabetes mellitus:0.46), (ROFLUMILAST:Type 1 diabetes mellitus:1.18), (SACCHAROMYCES CEREVISIAE:Type 1 diabetes mellitus:0.33), (SALMETEROL:Type 1 diabetes mellitus:0.23), (SALMETEROL XINAFOATE:Type 1 diabetes mellitus:0.25), (SODIUM PHOSPHATE, DIBASIC\SODIUM PHOSPHATE, MONOBASIC:Type 1 diabetes mellitus:0.34), (TERBINAFINE HYDROCHLORIDE:Type 1 diabetes mellitus:0.62), (TERBUTALINE:Type 1 diabetes mellitus:0.41), (TERBUTALINE SULFATE:Type 1 diabetes mellitus:1.05), (THALIDOMIDE:Type 1 diabetes mellitus:1.28), (TIGECYCLINE:Type 1 diabetes mellitus:0.05), (TRASTUZUMAB:Type 1 diabetes mellitus:0.13), (TRIAMCINOLONE:Type 1 diabetes mellitus:0.53), (UMECLIDINIUM BROMIDE:Type 1 diabetes mellitus:0.28), (VEMURAFENIB:Type 1 diabetes mellitus:0), (ZAFIRLUKAST:Type 1 diabetes mellitus:0.26), (ZONISAMIDE:Type 1 diabetes mellitus:0.61), (.BETA.-ACETYLDIGOXIN:Type 2 diabetes mellitus:1.42), (.BETA.-HYDROXYISOVALERIC ACIDARGININEGLUTAMINE:Type 2 diabetes mellitus:0.11), (ABOBOTULINUMTOXINA:Type 2 diabetes mellitus:1.16), (ACAMPROSATE:Type 2 diabetes mellitus:1.08), (ACETAMINOPHENAPRONALIDECAFFEINEPROPYPHENAZONE:Type 2 diabetes mellitus:1.17), (ACETAMINOPHENBROMISOVALDIHYDROCODEINE PHOSPHATEDIPHENHYDRAMINE SALICYLATEDYPHYLLINEMETHYLEPHEDRINE HYDROCHLORIDE, (+/−)-:Type 2 diabetes mellitus:0.29), (ACETAMINOPHENBUTALBITALCAFFEINECODEINE PHOSPHATE:Type 2 diabetes mellitus:0.51), (ACETAMINOPHEN\CAFFEINECARISOPRODOLDICLOFENAC:Type 2 diabetes mellitus:0.54), (ACETAMINOPHENCHLORPHENIRAMINE MALEATEPSEUDOEPHEDRINE HYDROCHLORIDE:Type 2 diabetes mellitus:0.22), (ACETAMINOPHENMETHOCARBAMOL:Type 2 diabetes mellitus:1.45), (ACETAMINOPHENPSEUDOEPHEDRINE HYDROCHLORIDE:Type 2 diabetes mellitus:0.21), (ACETIC ACIDSODIUM ACETATE:Type 2 diabetes mellitus:0.3), (ACETYLCYSTEINEBENZALKONIUM CHLORIDETUAMINOHEPTANE SULFATE:Type 2 diabetes mellitus:0.39), (ACOTIAMIDE HYDROCHLORIDE:Type 2 diabetes mellitus:0.11), (ADENINE:Type 2 diabetes mellitus:1.82), (AGALSIDASE BETA:Type 2 diabetes mellitus:3.21), (ALBUTEROLGUAIFENESIN:Type 2 diabetes mellitus:0.08), (ALBUTEROLIPRATROPIUM BROMIDE:Type 2 diabetes mellitus:0.27), (ALCLOMETASONE:Type 2 diabetes mellitus:0.01), (ALDESLEUKIN:Type 2 diabetes mellitus:0.65), (ALDIOXA:Type 2 diabetes mellitus:0.23), (ALECTINIB:Type 2 diabetes mellitus:0.28), (ALFALFA:Type 2 diabetes mellitus:0.32), (ALISPORIVIR:Type 2 diabetes mellitus:0), (ALUMINUM SILICATE:Type 2 diabetes mellitus:0.93), (AMANTADINE SULFATE:Type 2 diabetes mellitus:0.48), (AMINO ACIDSCARBOHYDRATESELECTROLYTES NOSVITAMINS:Type 2 diabetes mellitus:0.79), (AMINO ACIDSCARBOHYDRATESMINERALSSOYBEAN OILVITAMINS:Type 2 diabetes mellitus:1.07), (AMINO ACIDSDEXTROSEELECTROLYTES NOSSOYBEAN OIL:Type 2 diabetes mellitus:0.85), (AMOBARBITAL SODIUM:Type 2 diabetes mellitus:0.1), (AMOXICILLIN SODIUM:Type 2 diabetes mellitus:0.45), (AMOXICILLINCLARITHROMYCINLANSOPRAZOLE:Type 2 diabetes mellitus:0.86), (ANHYDROUS CITRIC ACIDMAGNESIUM OXIDE\SODIUM PICOSULFATE:Type 2 diabetes mellitus:0.19), (APOMORPHINE:Type 2 diabetes mellitus:0.23), (ARGATROBAN:Type 2 diabetes mellitus:0.58), (AROTINOLOL:Type 2 diabetes mellitus:0.94), (ASIAN GINSENG:Type 2 diabetes mellitus:1.03), (ASPIRINBUTALBITALCAFFEINECODEINE PHOSPHATE:Type 2 diabetes mellitus:0.12), (ATOMOXETINE:Type 2 diabetes mellitus:0.37), (ATROPINE SULFATEDIPHENOXYLATE:Type 2 diabetes mellitus:0.11), (AVOBENZONEHOMOSALATEOCTISALATE:Type 2 diabetes mellitus:0.73), (AVOCADO OILSOYBEAN OIL:Type 2 diabetes mellitus:1.07), (AZITHROMYCIN:Type 2 diabetes mellitus:0.07), (BACILLUS CALMETTE-GUERIN SUBSTRAIN CONNAUGHT LIVE ANTIGEN:Type 2 diabetes mellitus:0.23), (BACITRACINNEOMYCIN SULFATE:Type 2 diabetes mellitus:0.09), (BALSAM PERU OILCASTOR OILTRYPSIN:Type 2 diabetes mellitus:0.04), (BAZEDOXIFENE ACETATEESTROGENS, CONJUGATED:Type 2 diabetes mellitus:0.87), (BELATACEPT:Type 2 diabetes mellitus:1.19), (BENZALKONIUM CHLORIDE:Type 2 diabetes mellitus:0.02), (BEPRIDIL:Type 2 diabetes mellitus:3.26), (BEPRIDIL HYDROCHLORIDE:Type 2 diabetes mellitus:0.37), (BETAMETHASONE DIPROPIONATEBETAMETHASONE SODIUM PHOSPHATE:Type 2 diabetes mellitus:0.18), (BETAMETHASONE DIPROPIONATEGENTAMICIN SULFATE:Type 2 diabetes mellitus:0.15), (BEXAROTENE:Type 2 diabetes mellitus:0.1), (LONGUMLACTOBACILLUS ACIDOPHILUS:Type 2 diabetes mellitus:0.06), (BISACODYL OR GLYCERIN OR MINERAL OIL OR SODIUM PHOSPHATE, DIBASIC\SODIUM PHOSPHATE, MONOBASIC:Type 2 diabetes mellitus:0.13), (BISMUTH SUBCITRATE POTASSIUMMETRONIDAZOLETETRACYCLINE HYDROCHLORIDE:Type 2 diabetes mellitus:0.79), (BORNAPRINE HYDROCHLORIDE:Type 2 diabetes mellitus:0.02), (BOSUTINIB:Type 2 diabetes mellitus:1.38), (BUPIVACAINEEPINEPHRINE:Type 2 diabetes mellitus:0.04), (BUPRENORPHINENALOXONE:Type 2 diabetes mellitus:0.42), (BUPROPION HYDROBROMIDE:Type 2 diabetes mellitus:0.14), (BUTORPHANOL:Type 2 diabetes mellitus:0.23), (CABOZANTINIB:Type 2 diabetes mellitus:1.01), (CAFFEINEDIPYRONEORPHENADRINE:Type 2 diabetes mellitus:0.36), (CAFFEINEPHENOBARBITAL:Type 2 diabetes mellitus:0.05), (CAJUPUT OILEUCALYPTUS OILPEPPERMINT OIL:Type 2 diabetes mellitus:0.48), (CALCIUM BICARBONATE:Type 2 diabetes mellitus:0.73), (CALCIUM CARBONATEETIDRONATE DISODIUM:Type 2 diabetes mellitus:0.77), (CALCIUM CARBONATEMAGNESIUM CARBONATEMAGNESIUM TRISILICATE:Type 2 diabetes mellitus:0.63), (CALCIUM CHLORIDEDEXTROSE\POTASSIUM CHLORIDE\SODIUM ACETATE\SODIUM CHLORIDE:Type 2 diabetes mellitus:0.74), (CALCIUM CHLORIDE\POTASSIUM CHLORIDE\SODIUM CHLORIDE\SODIUM LACTATESORBITOL:Type 2 diabetes mellitus:0.11), (CALCIUM GLYCEROPHOSPHATE:Type 2 diabetes mellitus:0.22), (CALCIUMERGOCALCIFEROL:Type 2 diabetes mellitus:0.06), (CAMPHOR (NATURAL)MENTHOLPRAMOXINE HYDROCHLORIDE:Type 2 diabetes mellitus:0.23), (CAMPHORDIPHENHYDRAMINE\ZINC OXIDE:Type 2 diabetes mellitus:0.55), (CAMPHORMENTHOLMETHYL SALICYLATE:Type 2 diabetes mellitus:0.82), (CANNABIDIOL:Type 2 diabetes mellitus:0.69), (CARBAMIDE PEROXIDE:Type 2 diabetes mellitus:0.49), (CARIPRAZINE:Type 2 diabetes mellitus:0.21), (CARMUSTINE:Type 2 diabetes mellitus:0.61), (CARPRONIUM CHLORIDE:Type 2 diabetes mellitus:0.12), (CARTEOLOL:Type 2 diabetes mellitus:0.57), (CEFCAPENE PIVOXIL:Type 2 diabetes mellitus:0.05), (CEFOTIAM HYDROCHLORIDE:Type 2 diabetes mellitus:0.26), (CEPHARANTHINE:Type 2 diabetes mellitus:0.55), (CERIVASTATIN SODIUM:Type 2 diabetes mellitus:1.51), (CETYLPYRIDINIUM CHLORIDE OR SODIUM FLUORIDE OR SODIUM MONOFLUOROPHOSPHATE OR GLUCOSE OXIDASELACTOFERRINLACTOPEROXIDASE BOVINELYSOZYME:Type 2 diabetes mellitus:0.65), (CHLORAL HYDRATE:Type 2 diabetes mellitus:0.11), (CHLORMADINONE ACETATEETHINYL ESTRADIOL:Type 2 diabetes mellitus:0.59), (CHLORPROMAZINE HYDROCHLORIDEPHENOBARBITALPROMETHAZINE:Type 2 diabetes mellitus:0.41), (CHLORPROMAZINEPROMETHAZINE:Type 2 diabetes mellitus:0.59), (CHOLINE ALFOSCERATE:Type 2 diabetes mellitus:0.53), (CHOLINE BITARTRATECYSTEINEINOSITOLMAMMAL LIVER:Type 2 diabetes mellitus:0.65), (CHOLINETRAZODONE HYDROCHLORIDE:Type 2 diabetes mellitus:0.05), (CINNARIZINEDIMENHYDRINATE:Type 2 diabetes mellitus:0.02), (CIPROFLOXACINDEXAMETHASONE:Type 2 diabetes mellitus:0.33), (CISAPRIDE:Type 2 diabetes mellitus:0.75), (CITICOLINE SODIUM:Type 2 diabetes mellitus:0.78), (CLOMETHIAZOLE:Type 2 diabetes mellitus:0.77), (TETANICLOSTRIDIUM TETANI TOXOID ANTIGEN (FORMALDEHYDE INACTIVATED)DIPHTHERIAE TOXOID ANTIGEN (FORMALDEHYDE INACTIVATED):Type 2 diabetes mellitus:0.39), (COCOA BUTTERPETROLATUMPHENYLEPHRINE HYDROCHLORIDEPHENYLEPHRINE HYDROCHLORIDE OR MINERAL OIL:Type 2 diabetes mellitus:0), (CODEINE PHOSPHATEDICLOFENAC SODIUM:Type 2 diabetes mellitus:0.14), (CODEINE PHOSPHATEGUAIFENESINPSEUDOEPHEDRINE HYDROCHLORIDE:Type 2 diabetes mellitus:0.15), (CODEINE PHOSPHATEPROMETHAZINE HYDROCHLORIDE:Type 2 diabetes mellitus:0.19), (CODEINE SULFATE:Type 2 diabetes mellitus:0.29), (COLCHICINEOPIUMTIEMONIUM METHYLSULFATE:Type 2 diabetes mellitus:0.07), (CREATININE:Type 2 diabetes mellitus:0.07), (CROFELEMER:Type 2 diabetes mellitus:0.24), (CYANAMIDE:Type 2 diabetes mellitus:0.65), (CYANOCOBALAMINDICLOFENAC SODIUMPYRIDOXINE HYDROCHLORIDETHIAMINE MONONITRATE:Type 2 diabetes mellitus:0.16), (CYCLOPENTOLATE:Type 2 diabetes mellitus:0.21), (CYPROTERONE:Type 2 diabetes mellitus:0.85), (CYSTEINE:Type 2 diabetes mellitus:0.15), (CYSTINE:Type 2 diabetes mellitus:0.36), (DACARBAZINE:Type 2 diabetes mellitus:0.12), (DASABUVIR SODIUM MONOHYDRATE:Type 2 diabetes mellitus:0.01), (DASABUVIR SODIUM MONOHYDRATEOMBITASVIR HEMINONAHYDRATEPARITAPREVIR DIHYDRATERITONAVIR:Type 2 diabetes mellitus:0.13), (DENOPAMINE:Type 2 diabetes mellitus:0.17), (DESFLURANE:Type 2 diabetes mellitus:0.77), (DEXTRAN SULFATE SODIUM SALT:Type 2 diabetes mellitus:0.33), (DEXTROMETHORPHANDEXTROMETHORPHAN HYDROBROMIDEGUAIFENESIN:Type 2 diabetes mellitus:0.33), (DIACEREIN:Type 2 diabetes mellitus:0.41), (DIASTASE:Type 2 diabetes mellitus:0.25), (DIAZOXIDE:Type 2 diabetes mellitus:1.44), (DICLOFENAC DIETHYLAMINE:Type 2 diabetes mellitus:0.49), (DIDANOSINE:Type 2 diabetes mellitus:0.21), (DIENOGEST:Type 2 diabetes mellitus:0.32), (DIETARY SUPPLEMENTHERBALSMILK THISTLE:Type 2 diabetes mellitus:0.47), (DIFLUCORTOLONE VALERATELIDOCAINE:Type 2 diabetes mellitus:0.62), (DILAZEP DIHYDROCHLORIDE:Type 2 diabetes mellitus:0.33), (DIMEMORFAN PHOSPHATE:Type 2 diabetes mellitus:0.64), (DIMETHYL SULFOXIDE:Type 2 diabetes mellitus:0.22), (DORNASE ALFA:Type 2 diabetes mellitus:0.23), (DROSPIRENONE:Type 2 diabetes mellitus:0.09), (DUPILUMAB:Type 2 diabetes mellitus:0.12), (ECONAZOLE:Type 2 diabetes mellitus:3.48), (ELECTROLYTES NOSPOLYETHYLENE GLYCOL 3350:Type 2 diabetes mellitus:0.04), (EMTRICITABINERILPIVIRINE HYDROCHLORIDETENOFOVIR DISOPROXIL FUMARATE:Type 2 diabetes mellitus:0.36), (EPACADOSTAT:Type 2 diabetes mellitus:0.14), (EPHEDRINE:Type 2 diabetes mellitus:1.05), (ERGOLOID MESYLATES:Type 2 diabetes mellitus:0.85), (ERYTHROMYCIN LACTOBIONATE:Type 2 diabetes mellitus:0.68), (ESMOLOL HYDROCHLORIDE:Type 2 diabetes mellitus:1.77), (ESTRADIOL ACETATE:Type 2 diabetes mellitus:0.2), (ESTRADIOL BENZOATE:Type 2 diabetes mellitus:0.39), (ESTRADIOL HEMIHYDRATE:Type 2 diabetes mellitus:0.08), (ESTROGENS, ESTERIFIEDMETHYLTESTOSTERONE:Type 2 diabetes mellitus:0.76), (ESTROGENSMETHYLTESTOSTERONE:Type 2 diabetes mellitus:0.46), (ETHINYL ESTRADIOL:Type 2 diabetes mellitus:0.01), (ETHINYL ESTRADIOLFERROUS FUMARATENORETHINDRONE ACETATE:Type 2 diabetes mellitus:0.22), (ETHINYL ESTRADIOLGESTODENE:Type 2 diabetes mellitus:1.56), (ETHIONAMIDE:Type 2 diabetes mellitus:0.05), (ETIFOXINE:Type 2 diabetes mellitus:0.37), (ETOPOSIDE PHOSPHATE:Type 2 diabetes mellitus:0.2), (ETRETINATE:Type 2 diabetes mellitus:2.88), (EUCALYPTOLMENTHOLMETHYL SALICYLATETHYMOL:Type 2 diabetes mellitus:0.33), (FERRIC HYDROXIDE:Type 2 diabetes mellitus:2.67), (FERRIC POLYSACCHARIDE COMPLEX:Type 2 diabetes mellitus:0.23), (FERRITIN:Type 2 diabetes mellitus:0.32), (FERROGLYCINE SULFATE:Type 2 diabetes mellitus:0.35), (FIBRINOGEN HUMAN:Type 2 diabetes mellitus:0.1), (FILGRASTIM-SNDZ:Type 2 diabetes mellitus:0.24), (FLOMOXEF:Type 2 diabetes mellitus:0.19), (FLUMETHASONE PIVALATE:Type 2 diabetes mellitus:0.31), (FLUNARIZINE HYDROCHLORIDE:Type 2 diabetes mellitus:1.37), (FLUPENTIXOL DECANOATE:Type 2 diabetes mellitus:0.01), (FLUPENTIXOL DIHYDROCHLORIDE:Type 2 diabetes mellitus:0.25), (FLUPHENAZINE HYDROCHLORIDE:Type 2 diabetes mellitus:0.15), (FLURAZEPAM HYDROCHLORIDE:Type 2 diabetes mellitus:0.98), (FOSFOMYCIN CALCIUM:Type 2 diabetes mellitus:0.58), (FOSPHENYTOINFOSPHENYTOIN SODIUM:Type 2 diabetes mellitus:1.33), (FROVATRIPTAN SUCCINATE:Type 2 diabetes mellitus:1.06), (FUSIDATE SODIUM:Type 2 diabetes mellitus:0.07), (GADOBUTROL:Type 2 diabetes mellitus:0.16), (GADOPENTETATE DIMEGLUMINE:Type 2 diabetes mellitus:0.01), (GARENOXACIN MESYLATE:Type 2 diabetes mellitus:0.27), (GELATIN:Type 2 diabetes mellitus:0.34), (GENTIAN VIOLET:Type 2 diabetes mellitus:0.26), (GLATIRAMER:Type 2 diabetes mellitus:0.45), (GLUCONATE:Type 2 diabetes mellitus:0.32), (GLUTAMINE:Type 2 diabetes mellitus:1.09), (GLYCINE:Type 2 diabetes mellitus:0.02), (GREEN TEA LEAF:Type 2 diabetes mellitus:0.52), (HALCINONIDE:Type 2 diabetes mellitus:0.48), (HEPTAMINOL:Type 2 diabetes mellitus:0.33), (HERBALSMINERALSVITAMINS:Type 2 diabetes mellitus:0.18), (HERBSROOTS:Type 2 diabetes mellitus:0.17), (HIGH DENSITY POLYETHYLENE:Type 2 diabetes mellitus:0.43), (HORSE CHESTNUT:Type 2 diabetes mellitus:0.93), (HYDROCHLOROTHIAZIDEMETOPROLOL TARTRATE:Type 2 diabetes mellitus:1.03), (HYDROCORTISONE PROBUTATE:Type 2 diabetes mellitus:0.15), (HYDROQUINONE:Type 2 diabetes mellitus:0.25), (HYDROXYPROPYL CELLULOSE:Type 2 diabetes mellitus:0.67), (IMIGLUCERASE:Type 2 diabetes mellitus:1.99), (INOTUZUMAB OZOGAMICIN:Type 2 diabetes mellitus:0.8), (INTERFERON GAMMA-1B:Type 2 diabetes mellitus:0.69), (IODINE\POTASSIUM IODIDE:Type 2 diabetes mellitus:0.01), (IRON DEXTRAN:Type 2 diabetes mellitus:0.07), (ISOFLURANE:Type 2 diabetes mellitus:0.84), (ISOPROTERENOL HYDROCHLORIDE:Type 2 diabetes mellitus:0.13), (ISTRADEFYLLINE:Type 2 diabetes mellitus:0.56), (ITRACONAZOLE\SODIUM CHLORIDE:Type 2 diabetes mellitus:0.54), (KETAMINE HYDROCHLORIDE:Type 2 diabetes mellitus:1.19), (LAPATINIB:Type 2 diabetes mellitus:0.2), (LAPATINIB DITOSYLATE:Type 2 diabetes mellitus:1.39), (LECITHIN\SODIUM BICARBONATE\SODIUM PHOSPHATE, MONOBASIC:Type 2 diabetes mellitus:0.23), (LESINURAD:Type 2 diabetes mellitus:0.16), (LEVAMLODIPINE:Type 2 diabetes mellitus:0.1), (LEVOCABASTINE HYDROCHLORIDE:Type 2 diabetes mellitus:1.64), (LEVORPHANOL TARTRATE:Type 2 diabetes mellitus:0.05), (LEVOSIMENDAN:Type 2 diabetes mellitus:1.66), (LINSEED OIL:Type 2 diabetes mellitus:0.12), (LINSIDOMINE:Type 2 diabetes mellitus:1.78), (LOMUSTINE:Type 2 diabetes mellitus:0.34), (LOPINAVIR:Type 2 diabetes mellitus:0.8), (LUTETIUM LU-177:Type 2 diabetes mellitus:1.61), (LYSOZYME HYDROCHLORIDE:Type 2 diabetes mellitus:1.49), (MAGNESIUM PIDOLATE:Type 2 diabetes mellitus:0.94), (MAGNESIUM SULFATE\POTASSIUM SULFATE\SODIUM SULFATE:Type 2 diabetes mellitus:0.31), (MAPROTILINE HYDROCHLORIDE:Type 2 diabetes mellitus:0.92), (MENTHOLZINC OXIDE:Type 2 diabetes mellitus:0.75), (MEPENZOLATE BROMIDE:Type 2 diabetes mellitus:1.19), (METHENAMINE MANDELATE:Type 2 diabetes mellitus:1.11), (METHYLNALTREXONE BROMIDE:Type 2 diabetes mellitus:0.66), (METYRAPONE:Type 2 diabetes mellitus:0.81), (MIGLUSTAT:Type 2 diabetes mellitus:0.52), (MINERAL OILPETROLATUM:Type 2 diabetes mellitus:0.54), (MINERALSPROBIOTICS NOSVITAMINS:Type 2 diabetes mellitus:0.3), (MORPHINE\MORPHINE HYDROCHLORIDE\MORPHINE SULFATE\MORPHINE TARTRATE:Type 2 diabetes mellitus:0.69), (NEOSTIGMINE METHYLSULFATE:Type 2 diabetes mellitus:0.13), (NIFURTOINOL:Type 2 diabetes mellitus:0.65), (NIRAPARIB:Type 2 diabetes mellitus:1.71), (OK-432:Type 2 diabetes mellitus:0.06), (OLODATEROL:Type 2 diabetes mellitus:0.08), (OLODATEROLTIOTROPIUM BROMIDE:Type 2 diabetes mellitus:0.01), (OMACETAXINE:Type 2 diabetes mellitus:0.09), (OSTREA EDULIS SHELL:Type 2 diabetes mellitus:0.66), (OXANDROLONE:Type 2 diabetes mellitus:0.35), (OXETORONE:Type 2 diabetes mellitus:0.68), (OXYPERTINE:Type 2 diabetes mellitus:0.05), (PANCURONIUM:Type 2 diabetes mellitus:0.37), (PAPAYA:Type 2 diabetes mellitus:0.2), (PAZUFLOXACIN MESILATE:Type 2 diabetes mellitus:0.25), (PEGASPARGASE:Type 2 diabetes mellitus:0.38), (PENTOBARBITAL:Type 2 diabetes mellitus:0.15), (PENTOSTATIN:Type 2 diabetes mellitus:1.49), (PEPPERMINT OIL:Type 2 diabetes mellitus:0.03), (PEPSIN:Type 2 diabetes mellitus:0.26), (PERAZINE:Type 2 diabetes mellitus:0.19), (PHENAZEPAM:Type 2 diabetes mellitus:0.23), (PHENOXYBENZAMINE:Type 2 diabetes mellitus:0.26), (PHENYLBUTAZONE:Type 2 diabetes mellitus:0.08), (PHENYLEPHRINE HYDROCHLORIDEWITCH HAZEL:Type 2 diabetes mellitus:0.37), (PILSICAINIDE:Type 2 diabetes mellitus:0.78), (PIPERACILLIN SODIUM:Type 2 diabetes mellitus:0.35), (PODOFILOX:Type 2 diabetes mellitus:0.79), (POLYCARBOPHIL:Type 2 diabetes mellitus:0.39), (POLYETHYLENE GLYCOL 400:Type 2 diabetes mellitus:0.78), (PORFIMER SODIUM:Type 2 diabetes mellitus:0.5), (POTASSIUM CHLORIDE\SODIUM CHLORIDE:Type 2 diabetes mellitus:0.18), (POTASSIUM CHLORIDE\SODIUM CHLORIDE\SODIUM LACTATE:Type 2 diabetes mellitus:0.85), (POTASSIUM PHOSPHATE, MONOBASIC:Type 2 diabetes mellitus:0.92), (POVIDONE:Type 2 diabetes mellitus:0.89), (PRAMOXINE HYDROCHLORIDE:Type 2 diabetes mellitus:0.89), (PREDNISOLONE ACETATESULFACETAMIDE SODIUM:Type 2 diabetes mellitus:0.37), (PROPIOMAZINE:Type 2 diabetes mellitus:0.12), (PROPIVERINE HYDROCHLORIDE:Type 2 diabetes mellitus:0.65), (PROTEASE:Type 2 diabetes mellitus:0.24), (PROTEIN:Type 2 diabetes mellitus:0.12), (PROTRIPTYLINE HYDROCHLORIDE:Type 2 diabetes mellitus:1.68), (PRUCALOPRIDE SUCCINATE:Type 2 diabetes mellitus:1.31), (PSORALEN:Type 2 diabetes mellitus:0.3), (PYRIDOXAL:Type 2 diabetes mellitus:1.49), (PYRIDOXINE HYDROCHLORIDETHIAMINE HYDROCHLORIDE:Type 2 diabetes mellitus:0.23), (PYRIMETHAMINE:Type 2 diabetes mellitus:0.71), (QUERCUS SALICINA STEM:Type 2 diabetes mellitus:0.34), (QUINACRINE:Type 2 diabetes mellitus:1.51), (QUINAGOLIDE HYDROCHLORIDE:Type 2 diabetes mellitus:0.98), (RANITIDINE BISMUTH CITRATE:Type 2 diabetes mellitus:0.36), (REGADENOSON:Type 2 diabetes mellitus:0.51), (SAFINAMIDE:Type 2 diabetes mellitus:0.11), (SAFINAMIDE MESYLATE:Type 2 diabetes mellitus:0.32), (SAPROPTERIN DIHYDROCHLORIDE:Type 2 diabetes mellitus:0.23), (SARGRAMOSTIM:Type 2 diabetes mellitus:0.19), (SARPOGRELATE HYDROCHLORIDE:Type 2 diabetes mellitus:1.5), (SEA WATER:Type 2 diabetes mellitus:0.03), (SELEGILINE:Type 2 diabetes mellitus:2.1), (SENNOSIDE A:Type 2 diabetes mellitus:0.2), (SENNOSIDE ASENNOSIDE BSENNOSIDES:Type 2 diabetes mellitus:0.4), (SENNOSIDE B:Type 2 diabetes mellitus:0.77), (SERELAXIN:Type 2 diabetes mellitus:0.32), (SERTINDOLE:Type 2 diabetes mellitus:0.16), (SODIUM BICARBONATE\SODIUM CHLORIDE:Type 2 diabetes mellitus:0.6), (SODIUM CITRATE:Type 2 diabetes mellitus:1.66), (SODIUM PHOSPHATE, DIBASIC:Type 2 diabetes mellitus:0.61), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Type 2 diabetes mellitus:0.43), (SODIUM PHOSPHATE, MONOBASIC, ANHYDROUS:Type 2 diabetes mellitus:0.43), (SUCCINYLCHOLINE CHLORIDE:Type 2 diabetes mellitus:1.19), (SULFACETAMIDE SODIUM:Type 2 diabetes mellitus:0.06), (SUPROFEN:Type 2 diabetes mellitus:0.09), (TABALUMAB:Type 2 diabetes mellitus:0.34), (TALIGLUCERASE ALFA:Type 2 diabetes mellitus:0), (TAZAROTENE:Type 2 diabetes mellitus:0.27), (TEDUGLUTIDE:Type 2 diabetes mellitus:1.24), (TEDUGLUTIDE\WATER:Type 2 diabetes mellitus:1.95), (TEGAFURURACIL:Type 2 diabetes mellitus:0.66), (TELOTRISTAT ETHYL:Type 2 diabetes mellitus:1.56), (TENECTEPLASE:Type 2 diabetes mellitus:0.33), (TETRAZEPAM:Type 2 diabetes mellitus:0.74), (THIETHYLPERAZINE MALATE:Type 2 diabetes mellitus:0.04), (THIOPENTAL:Type 2 diabetes mellitus:1.26), (THIOTHIXENE HYDROCHLORIDE:Type 2 diabetes mellitus:0.16), (TIANEPTINE SODIUM:Type 2 diabetes mellitus:0.16), (TIARAMIDE:Type 2 diabetes mellitus:0.72), (TOFISOPAM:Type 2 diabetes mellitus:1.07), (TOPOTECAN HYDROCHLORIDE:Type 2 diabetes mellitus:0.05), (TOREMIFENE CITRATE:Type 2 diabetes mellitus:0.57), (TRANYLCYPROMINE:Type 2 diabetes mellitus:0.57), (TRANYLCYPROMINE SULFATE:Type 2 diabetes mellitus:0.18), (TREPROSTINIL DIOLAMINE:Type 2 diabetes mellitus:0.88), (TRYPTOPHAN:Type 2 diabetes mellitus:0.05), (TULOBUTEROL HYDROCHLORIDE:Type 2 diabetes mellitus:0.26), (URACIL:Type 2 diabetes mellitus:0.22), (UROKINASE:Type 2 diabetes mellitus:1.32), (VALRUBICIN:Type 2 diabetes mellitus:0.44), (VECURONIUM BROMIDE:Type 2 diabetes mellitus:1.24), (VELAGLUCERASE ALFA:Type 2 diabetes mellitus:0.52), (VINORELBINEVINORELBINE TARTRATE:Type 2 diabetes mellitus:0.45), (VONOPRAZAN:Type 2 diabetes mellitus:1.08), (WHEY:Type 2 diabetes mellitus:0.02), (ZANAMIVIR:Type 2 diabetes mellitus:0.13), (EMPAGLIFLOZINMETFORMIN HYDROCHLORIDE:Type 3 diabetes mellitus:0.07), (ACARBOSE:Type IIa hyperlipidaemia:0.7), (ACETAMINOPHENOXYCODONE HYDROCHLORIDEOXYCODONE TEREPHTHALATE:Type IIa hyperlipidaemia:0.68), (ACETAMINOPHENPROPOXYPHENE HYDROCHLORIDE:Type Ia hyperlipidaemia:0.2), (ACETAMINOPHENTRAMADOL:TypeIla hyperlipidaemia:0.06), (AGALSIDASE BETA:Type IIa hyperlipidaemia:0.16), (ALENDRONIC ACID:Type IIa hyperlipidaemia:0.01), (ALFACALCIDOL:Type IIa hyperlipidaemia:0.17), (AMANTADINE:Type IIa hyperlipidaemia:0.27), (AMLODIPINE BESYLATEATORVASTATIN CALCIUM:Type IIa hyperlipidaemia:0.08), (AMLODIPINEVALSARTAN:Type IIa hyperlipidaemia:0.09), (AMMONIUM LACTATE:Type IIa hyperlipidaemia:0.19), (BETAMETHASONE DIPROPIONATECALCIPOTRIENECALCIPOTRIENE HYDRATE:Type IIa hyperlipidaemia:0.24), (BRINZOLAMIDE:Type IIa hyperlipidaemia:0.49), (BUDESONIDEFORMOTEROL FUMARATE:Type IIa hyperlipidaemia:0.4), (BUPRENORPHINE HYDROCHLORIDENALOXONE HYDROCHLORIDE:Type IIa hyperlipidaemia:0.46), (CARFILZOMIB:Type IIa hyperlipidaemia:0.24), (CICLESONIDE:Type IIa hyperlipidaemia:0.27), (DACLIZUMAB:Type IIa hyperlipidaemia:1), (DARBEPOETIN ALFA:Type IIa hyperlipidaemia:0.31), (DASATINIB:Type IIa hyperlipidaemia:0.62), (DEFLAZACORT:Type IIa hyperlipidaemia:0.18), (DESONIDE:Type IIa hyperlipidaemia:0.24), (DEXTROAMPHETAMINE SULFATE:Type IIa hyperlipidaemia:0.2), (DIMENHYDRINATE:Type IIa hyperlipidaemia:0.54), (DOFETILIDE:Type IIa hyperlipidaemia:0.92), (DOXYCYCLINE HYCLATE:Type IIa hyperlipidaemia:0.03), (DRONABINOL:Type IIa hyperlipidaemia:0.34), (DUTASTERIDETAMSULOSIN HYDROCHLORIDE:Type IIa hyperlipidaemia:0.12), (EMPAGLIFLOZINLINAGLIPTIN:Type IIa hyperlipidaemia:1.21), (EMPAGLIFLOZINMETFORMIN HYDROCHLORIDE:Type IIa hyperlipidaemia:0.06), (ESTROGENS, CONJUGATEDMEDROXYPROGESTERONE ACETATE:Type IIa hyperlipidaemia:0.91), (ESTRONESUS SCROFA OVARY:Type IIa hyperlipidaemia:0.48), (FEXOFENADINEPSEUDOEPHEDRINE:Type IIa hyperlipidaemia:0.23), (FINGOLIMOD HYDROCHLORIDE:Type IIa hyperlipidaemia:0.11), (FLUDROCORTISONE ACETATE:Type IIa hyperlipidaemia:0.29), (FLUTICASONE FUROATE:Type IIa hyperlipidaemia:0.58), (FLUTICASONE FUROATESALMETEROL XINAFOATE:Type IIa hyperlipidaemia:0.2), (FORMOTEROL FUMARATE:Type IIa hyperlipidaemia:0.33), (FUSIDIC ACID:Type IIa hyperlipidaemia:0.03), (GLYCOPYRRONIUMINDACATEROL:Type IIa hyperlipidaemia:0.27), (HYDROCHLOROTHIAZIDEIRBESARTAN:Type IIa hyperlipidaemia:0.8), (HYDROCODONE BITARTRATEIBUPROFEN:Type IIa hyperlipidaemia:0.25), (HYDROXYCHLOROQUINE DIPHOSPHATE:Type IIa hyperlipidaemia:0.16), (IBRUTINIB:Type IIa hyperlipidaemia:0.74), (ILOPROST:Type IIa hyperlipidaemia:0.49), (IMIPRAMINE HYDROCHLORIDE:Type IIa hyperlipidaemia:0.8), (LACTASE:Type IIa hyperlipidaemia:0.21), (LERCANIDIPINE:Type IIa hyperlipidaemia:1.06), (LERCANIDIPINE HYDROCHLORIDE:Type IIa hyperlipidaemia:0.48), (LIDOCAINE HYDROCHLORIDE:Type IIa hyperlipidaemia:0.86), (LITHIUM CARBONATE:Type IIa hyperlipidaemia:0.23), (METFORMIN HYDROCHLORIDEVILDAGLIPTIN:Type IIa hyperlipidaemia:0.03), (MIDODRINE:Type IIa hyperlipidaemia:0.37), (MIDODRINE HYDROCHLORIDE:Type IIa hyperlipidaemia:0.01), (MINOCYCLINEMINOCYCLINE HYDROCHLORIDE:Type IIa hyperlipidaemia:0.39), (MOXIFLOXACIN HYDROCHLORIDE:Type IIa hyperlipidaemia:0.94), (NALOXONE HYDROCHLORIDEOXYCODONE HYDROCHLORIDE:Type IIa hyperlipidaemia:0.55), (NATEGLINIDE:Type Ia hyperlipidaemia:0.47), (NILOTINIB:Type IIa hyperlipidaemia:0.28), (NINTEDANIB:Type IIa hyperlipidaemia:0.23), (ONABOTULINUMTOXINA:Type IIa hyperlipidaemia:0.03), (OXYBUTYNIN:Type IIa hyperlipidaemia:0.58), (OXYBUTYNINOXYBUTYNIN CHLORIDE:Type IIa hyperlipidaemia:0.05), (PAZOPANIB HYDROCHLORIDE:Type IIa hyperlipidaemia:0.1), (PEGINTERFERON BETA-1A:Type IIa hyperlipidaemia:0.68), (PIMECROLIMUS:Type IIa hyperlipidaemia:0.06), (PONATINIB:Type IIa hyperlipidaemia:0.37), (PRAZOSIN HYDROCHLORIDE:Type IIa hyperlipidaemia:0.31), (PSEUDOEPHEDRINE HYDROCHLORIDE:Type IIa hyperlipidaemia:0.05), (PYRIDOSTIGMINE BROMIDE:Type IIa hyperlipidaemia:0.15), (PYRIDOXINE:Type IIa hyperlipidaemia:0.02), (QUETIAPINE:Type IIa hyperlipidaemia:0.64), (QUINAPRIL:Type IIa hyperlipidaemia:0.07), (QUINAPRILQUINAPRIL HYDROCHLORIDE:Type IIa hyperlipidaemia:0.19), (ROSIGLITAZONE MALEATE:Type IIa hyperlipidaemia:0.05), (SACCHAROMYCES CEREVISIAE:Type IIa hyperlipidaemia:0.06), (SALMETEROL XINAFOATE:Type IIa hyperlipidaemia:0.19), (SCOPOLAMINE:Type IIa hyperlipidaemia:0.34), (SECUKINUMAB:Type IIa hyperlipidaemia:0.37), (SODIUM PHOSPHATE, DIBASIC\SODIUM PHOSPHATE, MONOBASIC:Type IIa hyperlipidaemia:0.04), (SULFAMETHOXAZOLE:Type IIa hyperlipidaemia:0.12), (TERAZOSIN HYDROCHLORIDE:Type IIa hyperlipidaemia:0.1), (TETRACYCLINE:Type IIa hyperlipidaemia:1.27), (TOCILIZUMAB:Type IIa hyperlipidaemia:0.18), (UMECLIDINIUM BROMIDE:Type IIa hyperlipidaemia:0.06), (ALBUTEROL SULFATEIPRATROPIUM BROMIDE:Type V hyperlipidaemia:0.26), (AMPHETAMINE ASPARTATEAMPHETAMINE SULFATEDEXTROAMPHETAMINE SACCHARATEDEXTROAMPHETAMINE SULFATE:Type V hyperlipidaemia:0.19), (BISOPROLOL FUMARATE:Type V hyperlipidaemia:0.54), (CANDESARTAN:Type V hyperlipidaemia:0.32), (CANDESARTAN CILEXETIL:Type V hyperlipidaemia:0.13), (CANDESARTAN CILEXETILHYDROCHLOROTHIAZIDE:Type V hyperlipidaemia:0.01), (CARVEDILOL PHOSPHATE:Type V hyperlipidaemia:0.19), (CLINDAMYCINCLINDAMYCIN PHOSPHATE:Type V hyperlipidaemia:0.49), (DALFAMPRIDINE:Type V hyperlipidaemia:0.11), (DAPAGLIFLOZIN PROPANEDIOL:Type V hyperlipidaemia:0.32), (DASATINIB:Type V hyperlipidaemia:0.04), (DICLOFENAC:Type V hyperlipidaemia:0.07), (DILTIAZEM HYDROCHLORIDE:Type V hyperlipidaemia:1.27), (ENALAPRIL MALEATE:Type V hyperlipidaemia:0.11), (ERYTHROPOIETIN:Type V hyperlipidaemia:0.02), (ESZOPICLONE:Type V hyperlipidaemia:0.08), (FLECAINIDE:Type V hyperlipidaemia:0.16), (GLYBURIDE:Type V hyperlipidaemia:0.31), (HEPARIN SODIUM:Type V hyperlipidaemia:0.18), (HYOSCYAMINE:Type V hyperlipidaemia:0.02), (IBRUTINIB:Type V hyperlipidaemia:0.93), (ILOPROST:Type V hyperlipidaemia:0.17), (IRON:Type V hyperlipidaemia:0.61), (METHYLPREDNISOLONE SODIUM SUCCINATE:Type V hyperlipidaemia:0.36), (NAPROXEN SODIUM:Type V hyperlipidaemia:0.13), (PRAMIPEXOLE DIHYDROCHLORIDE:Type V hyperlipidaemia:0.11), (SODIUM OXYBATE:Type V hyperlipidaemia:0.1), (TETRACYCLINE:Type V hyperlipidaemia:0.08), (UMECLIDINIUM BROMIDEVILANTEROL TRIFENATATE:Type V hyperlipidaemia:0.05), (.ALPHA.1-PROTEINASE INHIBITOR HUMAN:Ulcer:0.73), (ACARBOSE:Ulcer:0.81), (ACETAMINOPHEN\CAFFEINEOPIUM:Ulc er:0.16), (ADO-TRASTUZUMAB EMTANSINE:Ulcer:0.65), (ALCOHOL:Ulcer:0.7), (ALEMTUZUMAB:Ulcer:0.16), (ALISKIRE N:Ulcer:0.34), (ALISKIREN HEMIFUMARATE:Ulcer:0.12), (AMANTADINE HYDROCHLORIDE:Ulcer:0.34), (AMIKACIN SULFATE:Ulcer:0.04), (AMILORIDE HYDROCHLORIDEHYDROCHLOROTHIAZIDE:Ulcer:0.21), (AMINO ACIDSDEXTROSEELECTROLYTES NOSSOYBEAN OILVITAMINS:Ulcer:0.13), (AMISULPRIDE:Ulcer:0.79), (AMLODIPINE BESYLATEHYDROCHLOROTHIAZIDEOLMESARTAN MEDOXOMIL:Ulcer:0.53), (AMLODIPINEPERINDOPRIL:Ulcer:0.21), (AMMONIUM LACTATE:Ulcer:0.16), (AMPHOTERICIN B:Ulcer:1.51), (AMPICILLIN:Ulcer:0.62), (ANTIHISTAMINES NOS:Ulcer:0.02), (ATOVAQUONE:Ulcer:0.6), (ATROPINEDIPHENOXYLATE:Ulcer:0.1), (A ZILSARTAN KAMEDOXOMIL:Ulcer:0.22), (BECLOMETHASONEFORMOTEROL:Ulcer:0.22), (BENAZ EPRILBENAZEPRIL HYDROCHLORIDE:Ulcer:0.8), (BENDAMUSTINE HYDROCHLORIDE:Ulcer:0.27), (BETAMETHASONE DIPROPIONATE:Ulcer:0.19), (BETAMETHASONE DIPROPIONATECLOTRIMAZOLE:Ulcer:0.02), (BETAMETHASONECALCIPOTRIENE:Ul cer:0.03), (BUDESONIDEFORMOTEROL FUMARATE:Ulcer:0.92), (CALCIPOTRIENE:Ulcer:0.08), (CALCITONIN SALMON:Ulcer:0.18), (CALCIUM\MAGNESIUM:Ulcer:0.11), (CALCIUM\MAGNESIUMZI NC:Ulcer:0.11), (CANAGLIFLOZINMETFORMIN HYDROCHLORIDE:Ulcer:0.57), (CANAKINUMAB:Ulcer:0.43), (CARBIDOPA:Ulcer:0.01), (CARBIMAZOLE:Ulcer:0.04), (CARVEDILOL PHOSPHATE:Ulcer:1.49), (CASPOFUNGIN:Ulcer:0.18), (CEFAZOLIN:Ulcer:0.41), (CETIRIZI NE HYDROCHLORIDEPSEUDOEPHEDRINE HYDROCHLORIDE:Ulcer:0.2), (CHLORHEXIDINE GLUCONATE:Ulcer:0.01), (CORTICOTROPIN:Ulcer:0.47), (CYPROHEPTADINE:Ulcer:0.19), (DABRAFENIB:Ulcer:0.21), (DACLIZUMAB:Ulcer:1.32), (DANAZOL:Ulcer:0.15), (DAPAGL IFLOZIN PROPANEDIOL:Ulcer:0.67), (DASABUVIROMBITASVIRPARITAPREVIRRITONAVIR:U lcer:0.09), (DESONIDE:Ulcer:0.39), (DEXAMETHASONE SODIUM PHOSPHATE:Ulcer:0), (DIGITOXIN:Ulcer:0.39), (DOCUSATE:Ulcer:1.35), (DOXEPIN HYDROCHLORIDE:Ulcer:0.61), (DOXYLAMINE SUCCINATE:Ulcer:0.05), (DUTASTERIDETAMSULOSIN HYDROCHLORIDE:Ulcer:0.18), (ECONAZOLE:Ulcer:0.71), (EDOXABAN TOSYLATE:Ulcer:1.01), (EPIRUBICIN:Ulcer:0.3), (ESTROGENS, CONJUGATEDMEDROXYPROGESTERONE ACETATE:Ulcer:0.63), (EVOLOCUMAB:Ulcer:1.62), (FLURAZEPAMFLURAZEPAM HYDROCHLORIDE:Ulcer:0.09), (FLUVOXAMINE MALEATE:Ulcer:0.35), (FORMOTEROL FUMARATE\MOMETASONE FUROATE:Ulcer:0.62), (FOSFOMYCIN:Ulcer:0.14), (GLYCOPYRROLATEINDACATEROL MALEATE:Ulcer:0.06), (GLYCOPYRRONIUM:Ulcer:0.37), (GLYCOPYRRONIUMINDACA TEROL:Ulcer:1.11), (GOSERELIN ACETATE:Ulcer:0.07), (HYDROCHLOROTHIAZIDEIRBESARTAN:Ulcer:1.06), (HYDROC ODONE BITARTRATEIBUPROFEN:Ulcer:0.2), (IBRITUMOMAB TIUXETAN:Ulcer:0.01), (ILOPROST:Ulcer:0.48), (INFLUENZA VIRUS VACCINE:Ulcer:0.89), (INSULIN BEEF:Ulcer:0.03), (INTERFERON ALFA-2A:Ulcer:0.21), (INTERFERON ALFA-2B:Ulcer:0.29), (IRON SUCROSE:Ulcer:0.17), (IXAZOMIB:Ulcer:0.25), (LABETALOL HYDROCHLORIDE:Ulcer:0.34), (LACTASE:Ulcer:0.28), (LAPATINIB DITOSYLATE:Ulcer:0.16), (LEDIPASVIRSOFOSBUVIR:Ulcer:1.64), (LERCANIDIPINE HYDROCHLORIDE:Ulcer:1.17), (LEVALBUTEROL:Ulcer:0.05), (LITHIUM CARBONATE:Ulcer:1.54), (MEPERIDINE:Ulcer:0.54), (MEPOLIZUMAB:Ulcer:0.42), (METH ENAMINE HIPPURATE:Ulcer:1.02), (MINOCYCLINEMINOCYCLINE HYDROCHLORIDE:Ulcer:1.32), (MUPIROCINMUPIROCIN CALCIUM:Ulcer:0.64), (NALOXONE HYDROCHLORIDETILIDINE HYDROCHLORIDE:Ulcer:0.35), (NICORANDIL:Ulcer:0.19), (NINTEDANIB:Ulcer:0.82), (NI RAPARIB:Ulcer:0.31), (OCTREOTIDE ACETATE:Ulcer:0.03), (OLMESARTAN:Ulcer:0.38), (OLODATEROL HYDROCHLORIDETIOTROPIUM BROMIDE MONOHYDRATE:Ulcer:0.83), (ORPHENADRINE:Ulcer:0.07), (PAMIDRONATE DISODIUM:Ulcer:0.66), (PATIROMER:Ulcer:0.43), (PEGINTERFERON BETA-A:Ulcer:0.77), (PENTOSTATIN:Ulcer:0.03), (PHENPROCOUMON:Ulcer:0.26), (PILOCARPI NE:Ulcer:0.06), (PIROXICAM:Ulcer:0.78), (POLYETHYLENE GLYCOL 4000:Ulcer:0.37), (PREDNISOLONE ACETATE:Ulcer:0.89), (PREDNISOLONE SODIUM PHOSPHATE:Ulcer:0.14), (QUINAPRIL:Ulcer:0.15), (RADIATION THERAPY:Ulcer:0.63), (REGORAFENIB:Ulcer:0.38), (SCOPOLAMINE:Ulcer:1.13), (SECUKI NUMAB:Ulcer:1.12), (SODIUM FLUORIDE:Ulcer:0.55), (SODIUM PHOSPHATE, DIBASIC\SODIUM PHOSPHATE, MONOBASIC:Ulcer:0.12), (SULFAMETHOXAZOLE:Ulcer:0.84), (TERAZOSIN HYDROCHLORIDE:Ulcer:0.37), (TIGECYCLINE:Ulcer:0.32), (TIMOLOL MALEATE:Ulcer:0.11), (TRAMETINIB:Ulcer:0.09), (TRANDOLAPRIL:Ulcer:0.11), (TRIMIP RAMINE MALEATE:Ulcer:0.01), (TROSPIUM CHLORIDE:Ulcer:0.37), (UMECLIDINIUM BROMIDE:Ulcer:0.14), (VILAZODONE HYDROCHLORIDE:Ulcer:0.69), (VINORELBINE TARTRATE:Ulcer:0.22), (ZAFIRLUKAST:Ulcer:0.21), (ZONISAMIDE:Ulcer:0.55), (EVOLOC UMAB:Ulcer haemorrhage:0.09), (FLUTICASONE PROPIONATE:Ulcer haemorrhage:0.39), (NAPROXEN:Ulcer haemorrhage:0.37), (RAMIPRIL:Ulcer haemorrhage:0.03), (ACETAMINOPHENOXYCODONE HYDROCHLORIDEOXYCODONE TEREPHTHALATE:Unevaluable event:0.15), (ACETAZOLAMIDE:Unevaluable event:0.21), (ALBIGLUTIDE:Unevaluable event:0.11), (AZITHROMYCIN ANHYDROUS:Unevaluable event:0.23), (BENAZEPRIL HYDROCHLORIDE:Unevaluable event:0.37), (BUPRENORPHINE HYDROCHLORIDENALOXONE HYDROCHLORIDE:Unevaluable event:0.54), (CALCIUM CARBONATEVITAMIN D:Unevaluable event:0.1), (CANAKINUMAB:Unevaluable event:0.08), (CANDESARTAN CILEXETILHYDROCHLOROTHIAZIDE:Unevaluable event:0.09), (CARFILZOMIB:Unevaluable event:0.41), (CARVEDILOL PHOSPHATE:Unevaluable event:0.31), (CINACALCET HYDROCHLORIDE:Unevaluable event:0.1), (DACLIZUMAB:Unevaluable event:0.39), (DESVENLAFAXINE SUCCINATE:Unevaluable event:0.51), (DIMENHYDRINATE:Unevaluable event:0.05), (DOCUSATE CALCIUM:Unevaluable event:0.09), (DOXAZOSIN MESYLATE:Unevaluable event:0.28), (DOXEPIN HYDROCHLORIDE:Unevaluable event:0.03), (DOXYCYCLINE:Unevaluable event:0.62), (DRONABINOL:Unevaluable event:0.04), (DUTASTERIDE:Unevaluable event:0.25), (ELETRIPTAN HYDROBROMIDE:Unevaluable event:1.02), (ENOXAPARIN:Unevaluable event:1.19), (ERYTHROMYCIN:Unevaluable event:0.02), (FESOTERODINE FUMARATE:Unevaluable event:0.26), (FISH OIL\OMEGA-3 FATTY ACIDS:Unevaluable event:0.03), (FLECAINIDE:Unevaluable event:0.06), (FLUTICASONE:Unevaluable event:0.62), (FLUTICASONE FUROATEVILANTEROL TRIFENATATE:Unevaluable event:0.49), (HALOPERIDOL:Unevaluable event:0.37), (HERBALSTURMERIC:Unevaluable event:0.06), (HYDROCHLOROTHIAZIDEVALSARTAN:Unevaluable event:0.24), (HYDROMORPHONE:Unevaluable event:0.45), (HYDROXYCHLOROQUINE DIPHOSPHATE:Unevaluable event:0.37), (IMATINIB MESYLATE:Unevaluable event:0.07), (INDAPAMIDE:Unevaluable event:0.1), (LABETALOLLABETALOL HYDROCHLORIDE:Unevaluable event:0.42), (LERCANIDIPINE:Unevaluable event:0.16), (LINAGLIPTIN:Unevaluable event:0.33), (LISDEXAMFETAMINE DIMESYLATE:Unevaluable event:0.83), (METHENAMINE HIPPURATE:Unevaluable event:0.12), (METHYLPREDNISOLONE ACETATE:Unevaluable event:0.57), (MINOCYCLINEMINOCYCLINE HYDROCHLORIDE:Unevaluable event:0.05), (NALOXONE HYDROCHLORIDEOXYCODONE HYDROCHLORIDE:Unevaluable event:0.05), (NEBIVOLOL:Unevaluable event:0.34), (OLANZAPINE:Unevaluable event:1.13), (PANTOPRAZOLE:Unevaluable event:1.63), (PANTOPRAZOLE MAGNESIUM:Unevaluable event:0.68), (PAROXETINE:Unevaluable event:0.4), (PEGFILGRASTIM:Unevaluable event:0.08), (PEGINTERFERON BETA-1A:Unevaluable event:0.35), (PENICILLIN:Unevaluable event:0.33), (PITAVASTATIN CALCIUM:Unevaluable event:0.04), (PREDNISOLONEPREDNISOLONE ACETATEPREDNISOLONE SODIUM SUCCINATE:Unevaluable event:0.02), (PYRIDOXINE:Unevaluable event:0), (RABEPRAZOLE:Unevaluable event:0.08), (ROFLUMILAST:Unevaluable event:0.12), (SACUBITRILVALSARTAN:Unevaluable event:0.58), (SCOPOLAMINE:Unevaluable event:0.36), (SECUKINUMAB:Unevaluable event:0.62), (SELEXIPAG:Unevaluable event:0.65), (SUMATRIPTAN:Unevaluable event:0.36), (TELMISARTAN:Unevaluable event:0.66), (TERIFLUNOMIDE:Unevaluable event:0.74), (TETRACYCLINE:Unevaluable event:0.2), (THALIDOMIDE:Unevaluable event:0.61), (TIMOLOL:Unevaluable event:0.26), (TRIAMTERENE:Unevaluable event:0.32), (UMECLIDINIUM BROMIDEVILANTEROL TRIFENATATE:Unevaluable event:0.63), (VALGANCICLOVIR HYDROCHLORIDE:Unevaluable event:0.17), (VARENICLINE TARTRATE:Unevaluable event:1.32), (VITAMIN B:Unevaluable event:0.97), (.ALPHA.1-PROTEINASE INHIBITOR HUMAN:Upper respiratory tract infection:0.99), (ACETAMINOPHENOXYCODONEOXYCODONE HYDROCHLORIDE:Upper respiratory tract infection:0.69), (ACETAMINOPHENPROPOXYPHENE NAPSYLATE:Upper respiratory tract infection:0.22), (AGALSIDASE BETA:Upper respiratory tract infection:0.18), (ALFUZOSIN HYDROCHLORIDE:Upper respiratory tract infection:0.08), (AMLODIPINEVALSARTAN:Upper respiratory tract infection:0.14), (ANAKINRA:Upper respiratory tract infection:0.03), (ASPIRIN LYSINE:Upper respiratory tract infection:0.51), (ATROPINE SULFATE:Upper respiratory tract infection:0.02), (BETAMETHASONECALCIPOTRIENE:Upper respiratory tract infection:0.08), (BIMATOPROST:Upper respiratory tract infection:0.64), (BLINATUMOMAB:Upper respiratory tract infection:0.09), (BUDESONIDEFORMOTEROL FUMARATE:Upper respiratory tract infection:0.89), (CABAZITAXEL:Upper respiratory tract infection:0.1), (CALCIPOTRIENE:Upper respiratory tract infection:0.21), (CALCITONIN SALMON:Upper respiratory tract infection:0.5), (CALCIUM ACETATE:Upper respiratory tract infection:0.84), (CALCIUM\MAGNESIUM:Upper respiratory tract infection:0.26), (CANAGLIFLOZINMETFORMIN HYDROCHLORIDE:Upper respiratory tract infection:0.49), (CANDESARTAN CILEXETILHYDROCHLOROTHIAZIDE:Upper respiratory tract infection:1.43), (CARVEDILOL PHOSPHATE:Upper respiratory tract infection:0.95), (CASPOFUNGIN:Upper respiratory tract infection:0.25), (CETUXIMAB:Upper respiratory tract infection:0.44), (CHLORPROMAZINE:Upper respiratory tract infection:0.14), (CLOSTRIDIUM BUTYRICUM SPORES STRAIN M-55:Upper respiratory tract infection:0.35), (CYPROHEPTADINE:Upper respiratory tract infection:0.52), (DABIGATRAN:Upper respiratory tract infection:0), (DACLIZUMAB:Upper respiratory tract infection:1.38), (DANAZOL:Upper respiratory tract infection:0.27), (DANTROLENE SODIUM:Upper respiratory tract infection:0), (DAPAGLIFLOZIN PROPANEDIOL:Upper respiratory tract infection:0.32), (DAPTOMYCIN:Upper respiratory tract infection:0.19), (DEFLAZACORT:Upper respiratory tract infection:0.69), (ECONAZOLE:Upper respiratory tract infection:0.5), (EDOXABAN TOSYLATE:Upper respiratory tract infection:0.66), (ELTROMBOPAG OLAMINE:Upper respiratory tract infection:0.33), (EMPAGLIFLOZINLINAGLIPTIN:Upper respiratory tract infection:0.37), (EPLERENONE:Upper respiratory tract infection:0.49), (EXEMESTANE:Upper respiratory tract infection:0.67), (FENOTEROL HYDROBROMIDEIPRATROPIUM BROMIDE:Upper respiratory tract infection:0.4), (FESOTERODINE FUMARATE:Upper respiratory tract infection:0.97), (FLUDROCORTISONE ACETATE:Upper respiratory tract infection:0.39), (FLUOCINOLONE ACETONIDE:Upper respiratory tract infection:0.04), (GIMERACILOTERACILTEGAFUR:Upper respiratory tract infection:0.31), (GLYCOPYRROLATE:Upper respiratory tract infection:0.76), (GLYCOPYRROLATEINDACATEROL MALEATE:Upper respiratory tract infection:0.04), (HERBALSTURMERIC:Upper respiratory tract infection:0.59), (HYDROXYCHLOROQUINE DIPHOSPHATE:Upper respiratory tract infection:0.44), (HYOSCYAMINE:Upper respiratory tract infection:0.39), (ILOPROST:Upper respiratory tract infection:0.67), (IMATINIB MESYLATE:Upper respiratory tract infection:1.2), (IMIPRAMINE HYDROCHLORIDE:Upper respiratory tract infection:1.01), (IRINOTECAN:Upper respiratory tract infection:0.26), (IVACAFTOR:Upper respiratory tract infection:0.31), (LACTASE:Upper respiratory tract infection:0.1), (LAPATINIB DITOSYLATE:Upper respiratory tract infection:0.37), (LEDIPASVIRSOFOSBUVIR:Upper respiratory tract infection:1.28), (LEVOLEUCOVORIN CALCIUM:Upper respiratory tract infection:0.3), (LUMACAFTOR:Upper respiratory tract infection:0.14), (MECLIZINEMECLIZINE HYDROCHLORIDE:Upper respiratory tract infection:0.22), (MEPERIDINE:Upper respiratory tract infection:0.06), (MEPOLIZUMAB:Upper respiratory tract infection:1.05), (METFORMIN HYDROCHLORIDEVILDAGLIPTIN:Upper respiratory tract infection:0.29), (METHENAMINE HIPPURATE:Upper respiratory tract infection:1.24), (METHENOLONE ENANTHATE:Upper respiratory tract infection:0.06), (METOLAZONE:Upper respiratory tract infection:0.36), (MIDODRINE:Upper respiratory tract infection:0.25), (NADOLOL:Upper respiratory tract infection:0.25), (NATEGLINIDE:Upper respiratory tract infection:0.15), (NIRAPARIB:Upper respiratory tract infection:0.26), (NORETHINDRONE:Upper respiratory tract infection:0.35), (OCTREOTIDE ACETATE:Upper respiratory tract infection:0.09), (OLAPARIB:Upper respiratory tract infection:0.46), (OLODATEROL HYDROCHLORIDETIOTROPIUM BROMIDE MONOHYDRATE:Upper respiratory tract infection:1.72), (OLOPATADINE:Upper respiratory tract infection:0.5), (PAZOPANIB HYDROCHLORIDE:Upper respiratory tract infection:1.41), (PEGINTERFERON ALFA-2B:Upper respiratory tract infection:0.01), (PEGINTERFERON BETA-1A:Upper respiratory tract infection:0.87), (PHENYTOIN SODIUM:Upper respiratory tract infection:0.53), (PIROXICAM:Upper respiratory tract infection:0.66), (PONATINIB:Upper respiratory tract infection:0.62), (POSACONAZOLE:Upper respiratory tract infection:1.32), (PRAMIPEXOLE:Upper respiratory tract infection:0.81), (PRIMIDONE:Upper respiratory tract infection:1.43), (PYRAZINAMIDE:Upper respiratory tract infection:0.75), (QUINAPRIL:Upper respiratory tract infection:0.03), (RADIATION THERAPY:Upper respiratory tract infection:0.39), (REGORAFENIB:Upper respiratory tract infection:0.41), (RIVASTIGMINE TARTRATE:Upper respiratory tract infection:0.07), (RIZATRIPTAN BENZOATE:Upper respiratory tract infection:1.09), (SALMETEROL:Upper respiratory tract infection:0.25), (SITAGLIPTIN:Upper respiratory tract infection:0.17), (SOTALOL:Upper respiratory tract infection:0.9), (SUMATRIPTANSUMATRIPTAN SUCCINATE:Upper respiratory tract infection:1.13), (TEMOZOLOMIDE:Upper respiratory tract infection:0.17), (TERBUTALINE:Upper respiratory tract infection:0.18), (TETRACYCLINE:Upper respiratory tract infection:0.68), (THEOPHYLLINE:Upper respiratory tract infection:0.32), (THYROID, PORCINE:Upper respiratory tract infection:0.1), (TICAGRELOR:Upper respiratory tract infection:0.37), (TIMOLOL MALEATE:Upper respiratory tract infection:0.39), (TINZAPARIN SODIUM:Upper respiratory tract infection:0.86), (TOCOPHEROL:Upper respiratory tract infection:0.09), (TOLTERODINE:Upper respiratory tract infection:0.14), (UMECLIDINIUM BROMIDE:Upper respiratory tract infection:0.45), (VALGANCICLOVIR HYDROCHLORIDE:Upper respiratory tract infection:1.2), (VANCOMYCIN HYDROCHLORIDE:Upper respiratory tract infection:1.43), (VINORELBINE TARTRATE:Upper respiratory tract infection:0.51), (VITAMIN A:Upper respiratory tract infection:0.7), (ZAFIRLUKAST:Upper respiratory tract infection:0.23), (ZILEUTON:Upper respiratory tract infection:0.24), (ZINC:Upper respiratory tract infection:0.74), (AMEZINIUM METILSULFATE:Upper respiratory tract inflammation:0.65), (AMOXAPINE:Upper respiratory tract inflammation:0.13), (BEPRIDIL:Upper respiratory tract inflammation:0.31), (BUCILLAMINE:Upper respiratory tract inflammation:0.76), (CALCIUM CARBONATECHOLECALCIFEROLMAGNESIUM CARBONATE:Upper respiratory tract inflammation:0.02), (CALCIUM LACTATE:Upper respiratory tract inflammation:0.05), (CAMPHORMENTHOLMETHYL SALICYLATE:Upper respiratory tract inflammation:0.68), (CARPRONIUM CHLORIDE:Upper respiratory tract inflammation:0.03), (CEPHARANTHINE:Upper respiratory tract inflammation:0.52), (DEPRODONE PROPIONATE:Upper respiratory tract inflammation:0), (DIPHENIDOL HYDROCHLORIDE:Upper respiratory tract inflammation:0.57), (EPERISONE HYDROCHLORIDE:Upper respiratory tract inflammation:0.33), (ETHYL ICOSAPENTATEICOSAPENT:Upper respiratory tract inflammation:0.23), (ETRETINATE:Upper respiratory tract inflammation:0.87), (FAROPENEM SODIUM:Upper respiratory tract inflammation:0.42), (FERRIC PYROPHOSPHATE:Upper respiratory tract inflammation:0.21), (GUANABENZ ACETATE:Upper respiratory tract inflammation:0.53), (HERBSROOTS:Upper respiratory tract inflammation:0.11), (IGURATIMOD:Upper respiratory tract inflammation:0.26), (IRSOGLADINE MALEATE:Upper respiratory tract inflammation:0.33), (LYSOZYME HYDROCHLORIDE:Upper respiratory tract inflammation:0.48), (METHENOLONE ENANTHATE:Upper respiratory tract inflammation:0.03), (POTASSIUM CITRATETRISODIUM CITRATE DIHYDRATE:Upper respiratory tract inflammation:0.25), (SUPLATAST TOSILATE:Upper respiratory tract inflammation:0.47), (TEMOCAPRIL HYDROCHLORIDE:Upper respiratory tract inflammation:0.64), (WARFARIN POTASSIUM:Upper respiratory tract inflammation:0.13), (ACETAMINOPHENBUTALBITALCAFFEINE:Upper-airway cough syndrome:0.11), (AMIODARONE:Upper-airway cough syndrome:0.1), (AMITRIPTYLINE:Upper-airway cough syndrome:0.32), (ANASTROZOLE:Upper-airway cough syndrome:0.33), (ARMODAFINIL:Upper-airway cough syndrome:0.3), (BUSPIRONE HYDROCHLORIDE:Upper-airway cough syndrome:0.52), (CEPHALEXIN:Upper-airway cough syndrome:0.01), (CHOLESTYRAMINE:Upper-airway cough syndrome:0.48), (CLINDAMYCINCLINDAMYCIN PHOSPHATE:Upper-airway cough syndrome:0.26), (CLONIDINE:Upper-airway cough syndrome:0.15), (CYCLOBENZAPRINE:Upper-airway cough syndrome:0.37), (DICYCLOMINE HYDROCHLORIDE:Upper-airway cough syndrome:0.24), (ELETRIPTAN HYDROBROMIDE:Upper-airway cough syndrome:0.2), (ERGOCALCIFEROL:Upper-airway cough syndrome:0.31), (ESZOPICLONE:Upper-airway cough syndrome:0.18), (EVOLOCUMAB:Upper-airway cough syndrome:0.41), (FERROUS SULFATE:Upper-airway cough syndrome:0.3), (IBRUTINIB:Upper-airway cough syndrome:0.67), (ILOPROST:Upper-airway cough syndrome:0.04), (INSULIN LISPRO:Upper-airway cough syndrome:0.6), (ISOSORBIDE:Upper-airway cough syndrome:0.07), (LISDEXAMFETAMINE DIMESYLATE:Upper-airway cough syndrome:0.24), (NAPROXEN:Upper-airway cough syndrome:1.02), (PANTOPRAZOLE MAGNESIUM:Upper-airway cough syndrome:0.54), (RANOLAZINE:Upper-airway cough syndrome:0.62), (SACUBITRILVALSARTAN:Upper-airway cough syndrome:0.28), (SUMATRIPTANSUMATRIPTAN SUCCINATE:Upper-airway cough syndrome:0.12), (TERIFLUNOMIDE:Upper-airway cough syndrome:0.52), (VARENICLINE TARTRATE:Upper-airway cough syndrome:0.52), (VERAPAMIL HYDROCHLORIDE:Upper-airway cough syndrome:0.49), (DILTIAZEM HYDROCHLORIDE:Urge incontinence:0.28), (ESCITALOPRAM OXALATE:Urge incontinence:0.3), (EVOLOCUMAB:Urge incontinence:0.38), (IBRUTINIB:Urge incontinence:0.53), (METHYLPREDNISOLONE SODIUM SUCCINATE:Urge incontinence:0.16), (NEBIVOLOL:Urge incontinence:0.13), (POTASSIUM:Urge incontinence:0.34), (PRAVASTATIN:Urge incontinence:0.02), (RABEPRAZOLE SODIUM:Urge incontinence:0.02), (RISEDRONATE SODIUM:Urge incontinence:0.03), (TERIFLUNOMIDE:Urge incontinence:0.38), (VENLAFAXINE HYDROCHLORIDE:Urge incontinence:0.16), (.ALPHA.1-PROTEINASE INHIBITOR HUMAN:Urinary incontinence:0.38), (ABOBOTULINUMTOXINA:Urinary incontinence:0), (ACARBOSE:Urinary incontinence:0.56), (ACETAZOLAMIDE:Urinary incontinence:1.07), (AGALSIDASE BETA:Urinary incontinence:0.49), (ALISKIREN:Urinary incontinence:0.17), (AMISULPRIDE:Urinary incontinence:0.62), (AMLODIPINE BESYLATEHYDROCHLOROTHIAZIDEVALSARTAN:Urinary incontinence:0.39), (AMLODIPINEVALSARTAN:Urinary incontinence:0.36), (APREPITANT:Urinary incontinence:0.18), (AXITINIB:Urinary incontinence:0.15), (BECLOMETHASONEFORMOTEROL:Urinary incontinence:0.03), (BETAMETHASONE DIPROPIONATE:Urinary incontinence:0.03), (BETAMETHASONE DIPROPIONATECALCIPOTRIENECALCIPOTRIENE HYDRATE:Urinary incontinence:0.16), (BETAMETHASONE DIPROPIONATECLOTRIMAZOLE:Urinary incontinence:0.05), (BETAMETHASONECALCIPOTRIENE:Urinary incontinence:0.03), (BETHANECHOL CHLORIDE:Urinary incontinence:0.1), (BUPRENORPHINE HYDROCHLORIDENALOXONE HYDROCHLORIDE:Urinary incontinence:0.63), (BUTYLSCOPOLAMINE BROMIDE:Urinary incontinence:0.06), (CALCIPOTRIENE:Urinary incontinence:0.32), (CALCITONIN:Urinary incontinence:0.33), (CALCIUM ACETATE:Urinary incontinence:1.08), (CALCIUM\MAGNESIUM\ZINC:Urinary incontinence:0.42), (CANAGLIFLOZINMETFORMIN HYDROCHLORIDE:Urinary incontinence:0.61), (CARVEDILOL PHOSPHATE:Urinary incontinence:1.48), (CEFUROXIME AXETIL:Urinary incontinence:0.17), (CETIRIZINE HYDROCHLORIDEPSEUDOEPHEDRINE HYDROCHLORIDE:Urinary incontinence:0.33), (CHONDROITIN SULFATE (BOVINE)GLUCOSAMINE HYDROCHLORIDE:Urinary incontinence:0.64), (CILASTATIN SODIUMIMIPENEM:Urinary incontinence:0.25), (CIMETIDINE:Urinary incontinence:1.09), (CINACALCET HYDROCHLORIDE:Urinary incontinence:0.07), (CLOBAZAM:Urinary incontinence:0.37), (CLOTRIMAZOLE:Urinary incontinence:1.12), (CODEINE PHOSPHATEGUAIFENESIN:Urinary incontinence:0.21), (DAPAGLIFLOZIN PROPANEDIOL:Urinary incontinence:0.84), (DASABUVIROMBITASVIRPARITAPREVIRRITONAVIR:Urinary incontinence:0), (DEXTROAMPHETAMINE SULFATE:Urinary incontinence:0.42), (DIGITOXIN:Urinary incontinence:0.17), (DIPYRONE:Urinary incontinence:0.03), (DORZOLAMIDE:Urinary incontinence:0.36), (DOXORUBICIN HYDROCHLORIDE:Urinary incontinence:0.05), (DUTASTERIDETAMSULOSIN HYDROCHLORIDE:Urinary incontinence:0.31), (ECONAZOLE:Urinary incontinence:0.44), (EDOXABAN TOSYLATE:Urinary incontinence:0.55), (EMPAGLIFLOZINLINAGLIPTIN:Urinary incontinence:0.55), (ENOXAPARIN:Urinary incontinence:2.22), (ESTROGENS:Urinary incontinence:0.1), (ETHINYL ESTRADIOLNORGESTIMATE:Urinary incontinence:0.03), (EVOLOCUMAB:Urinary incontinence:1.87), (FENOFIBRIC ACID:Urinary incontinence:0.47), (FLUDROCORTISONE:Urinary incontinence:0.21), (FLUDROCORTISONE ACETATE:Urinary incontinence:0.94), (FLURAZEPAMFLURAZEPAM HYDROCHLORIDE:Urinary incontinence:0.16), (FLUVOXAMINE MALEATE:Urinary incontinence:0.55), (FORMOTEROL:Urinary incontinence:0.29), (FORMOTEROL FUMARATE:Urinary incontinence:1.07), (FORMOTEROL FUMARATE\MOMETASONE FUROATE:Urinary incontinence:0.43), (FUSIDIC ACID:Urinary incontinence:0.59), (GLYCOPYRRONIUMINDACATEROL:Urinary incontinence:1.11), (GUANFACINE:Urinary incontinence:0.14), (GUANFACINE HYDROCHLORIDE:Urinary incontinence:0.38), (HYDROXYCHLOROQUINE DIPHOSPHATE:Urinary incontinence:0.46), (HYDROXYZINE PAMOATE:Urinary incontinence:2.02), (HYOSCYAMINE:Urinary incontinence:0.93), (HYOSCYAMINE SULFATE:Urinary incontinence:0.15), (INDACATEROL:Urinary incontinence:0.42), (INFLIXIMAB-DYYB:Urinary incontinence:0.18), (INSULIN GLULISINE:Urinary incontinence:0.47), (INTERFERON ALFA-2A:Urinary incontinence:0.09), (ISOSORBIDE DINITRATE:Urinary incontinence:0.16), (KETOPROFEN:Urinary incontinence:0.06), (LEVOMEFOLATE CALCIUM\METHYLCOBALAMINPYRIDOXAL PHOSPHATE ANHYDROUS:Urinary incontinence:0.02), (LIOTHYRONINE SODIUM:Urinary incontinence:0.57), (LOMITAPIDE MESYLATE:Urinary incontinence:0.58), (LOTEPREDNOL ETABONATE:Urinary incontinence:0.02), (MEDROXYPROGESTERONE:Urinary incontinence:0.07), (MEPOLIZUMAB:Urinary incontinence:0.28), (METHYLPHENIDATE:Urinary incontinence:0.01), (METHYLPREDNISOLONE ACETATE:Urinary incontinence:1.37), (MIDODRINE HYDROCHLORIDE:Urinary incontinence:0.26), (NORETHINDRONE:Urinary incontinence:0.17), (OLMESARTAN:Urinary incontinence:0.13), (OLODATEROL HYDROCHLORIDETIOTROPIUM BROMIDE MONOHYDRATE:Urinary incontinence:0.67), (PATIROMER:Urinary incontinence:0.2), (PAZOPANIB HYDROCHLORIDE:Urinary incontinence:0.96), (PEMETREXED DISODIUM:Urinary incontinence:0.59), (POLYETHYLENE GLYCOL 4000:Urinary incontinence:0.02), (PONATINIB:Urinary incontinence:0.89), (PREDNISOLONEPREDNISOLONE ACETATEPREDNISOLONE SODIUM SUCCINATE:Urinary incontinence:0.15), (PROPAFENONE:Urinary incontinence:0.09), (PYRAZINAMIDE:Urinary incontinence:0.71), (ROFLUMILAST:Urinary incontinence:0.87), (SACUBITRILVALSARTAN:Urinary incontinence:1.34), (SALMETEROL XINAFOATE:Urinary incontinence:0.27), (SELEGILINE:Urinary incontinence:0.22), (SITAGLIPTIN:Urinary incontinence:0.63), (SODIUM FLUORIDE:Urinary incontinence:0.14), (SODIUM PHOSPHATE, DIBASIC\SODIUM PHOSPHATE, MONOBASIC:Urinary incontinence:0.03), (SULINDAC:Urinary incontinence:0.03), (TERBUTALINE SULFATE:Urinary incontinence:1.19), (TESTOSTERONE UNDECANOATE:Urinary incontinence:0.22), (TETRACYCLINE:Urinary incontinence:1.13), (THALIDOMIDE:Urinary incontinence:0.92), (TOBRAMYCIN:Urinary incontinence:0.06), (TRIMIPRAMINE MALEATE:Urinary incontinence:0.22), (VALGANCICLOVIR HYDROCHLORIDE:Urinary incontinence:1.36), (VANCOMYCIN HYDROCHLORIDE:Urinary incontinence:0.01), (ZINC:Urinary incontinence:0.88), (.ALPHA.1-PROTEINASE INHIBITOR HUMAN:Urinary retention:0.21), (ACENOCOUMAROL:Urinary retention:0.32), (ACETAMINOPHENOXYCODONEOXYCODONE HYDROCHLORIDE:Urinary retention:0.38), (ACLIDINIUM BROMIDE:Urinary retention:0.04), (ALCOHOL:Urinary retention:0.29), (ALENDRONIC ACID:Urinary retention:0.12), (ALUMINUM HYDROXIDEDIMETHICONEMAGNESIUM HYDROXIDE:Urinary retention:0.05), (AMISULPRIDE:Urinary retention:0.11), (ANASTROZOLE:Urinary retention:1.1), (BENAZEPRILBENAZEPRIL HYDROCHLORIDE:Urinary retention:0.25), (BENZONATATE:Urinary retention:0.19), (BUDESONIDEFORMOTEROL FUMARATE:Urinary retention:0.25), (BUPRENORPHINE HYDROCHLORIDENALOXONE HYDROCHLORIDE:Urinary retention:0.07), (CANAGLIFLOZINMETFORMIN HYDROCHLORIDE:Urinary retention:0.14), (CARVEDILOL PHOSPHATE:Urinary retention:0.55), (CHLORTHALIDONE:Urinary retention:0.32), (CICLESONIDE:Urinary retention:0.25), (CIMETIDINE:Urinary retention:0.38), (CINACALCET HYDROCHLORIDE:Urinary retention:0.07), (DACLIZUMAB:Urinary retention:0.39), (DAPAGLIFLOZIN PROPANEDIOL:Urinary retention:0.24), (DASATINIB:Urinary retention:1.4), (DULAGLUTIDE:Urinary retention:0.02), (ECONAZOLE:Urinary retention:0.01), (EDOXABAN TOSYLATE:Urinary retention:0.15), (ELETRIPTAN HYDROBROMIDE:Urinary retention:0.54), (ELOTUZUMAB:Urinary retention:0.04), (EMPAGLIFLOZINLINAGLIPTIN:Urinary retention:0.03), (ENALAPRIL MALEATE:Urinary retention:0.97), (ERYTHROMYCIN:Urinary retention:0.16), (EVOLOCUMAB:Urinary retention:1.06), (FEBUXOSTAT:Urinary retention:0.78), (FISH OIL\OMEGA-3 FATTY ACIDS:Urinary retention:0.08), (FLUDROCORTISONE ACETATE:Urinary retention:0.12), (FLUTICASONE FUROATESALMETEROL XINAFOATE:Urinary retention:0.47), (GLYCOPYRRONIUMINDACATEROL:Urinary retention:0.53), (HERBALSTURMERIC:Urinary retention:0.15), (HYDROCHLOROTHIAZIDELISINOPRIL:Urinary retention:1.27), (HYDROCHLOROTHIAZIDETELMISARTAN:Urinary retention:0.11), (HYOSCYAMINE:Urinary retention:0.39), (ILOPROST:Urinary retention:0.3), (IMATINIB MESYLATE:Urinary retention:0.74), (INDAPAMIDEPERINDOPRIL:Urinary retention:0.78), (INTERFERON ALFA-2A:Urinary retention:0.05), (LEDIPASVIRSOFOSBUVIR:Urinary retention:1.06), (LERCANIDIPINE:Urinary retention:0.85), (LERCANIDIPINE HYDROCHLORIDE:Urinary retention:0.46), (METAMIZOLE:Urinary retention:0.06), (METHADONE HYDROCHLORIDE:Urinary retention:0.54), (METHIMAZOLE:Urinary retention:0.1), (METHYLPREDNISOLONE ACETATE:Urinary retention:0.31), (MINOCYCLINEMINOCYCLINE HYDROCHLORIDE:Urinary retention:0.06), (NADOLOL:Urinary retention:0.39), (NALOXONE HYDROCHLORIDEOXYCODONE HYDROCHLORIDE:Urinary retention:0.7), (NEBIVOLOL:Urinary retention:0.36), (NEBIVOLOL HYDROCHLORIDE:Urinary retention:0.82), (NILOTINIB:Urinary retention:1.34), (NIRAPARIB:Urinary retention:0.03), (NORTRIPTYLINE:Urinary retention:0.66), (OLODATEROL HYDROCHLORIDETIOTROPIUM BROMIDE MONOHYDRATE:Urinary retention:0.34), (PAZOPANIB HYDROCHLORIDE:Urinary retention:0.4), (PEGINTERFERON BETA-1A:Urinary retention:0.41), (PHENYTOIN SODIUM:Urinary retention:0.03), (PIROXICAM:Urinary retention:0.11), (PONATINIB:Urinary retention:0.68), (POSACONAZOLE:Urinary retention:0.19), (PREDNISOLONE ACETATE:Urinary retention:0.17), (PREDNISOLONEPREDNISOLONE ACETATEPREDNISOLONE SODIUM SUCCINATE:Urinary retention:0.07), (RIVASTIGMINE TARTRATE:Urinary retention:0.03), (SUMATRIPTANSUMATRIPTAN SUCCINATE:Urinary retention:0.43), (TERBINAFINE HYDROCHLORIDE:Urinary retention:0.13), (TOCILIZUMAB:Urinary retention:0.11), (TOFACITINIB CITRATE:Urinary retention:0.2), (UMECLIDINIUM BROMIDEVILANTEROL TRIFENATATE:Urinary retention:0.54), (VALGANCICLOVIR HYDROCHLORIDE:Urinary retention:0.64), (VARENICLINE TARTRATE:Urinary retention:1.21), (VILAZODONE HYDROCHLORIDE:Urinary retention:0.07), (ACETAMINOPHENBUTALBITALCAFFEINE:Urinary tract disorder:0.76), (ADO-TRASTUZUMAB EMTANSINE:Urinary tract disorder:0.12), (AMISULPRIDE:Urinary tract disorder:0.06), (AMLODIPINE BESYLATEHYDROCHLOROTHIAZIDEOLMESARTAN MEDOXOMIL:Urinary tract disorder:0.27), (BORTEZOMIB:Urinary tract disorder:0.13), (BUDESONIDEFORMOTEROL FUMARATE:Urinary tract disorder:0.03), (CALCIUM ACETATE:Urinary tract disorder:0.13), (CANDESARTAN CILEXETILHYDROCHLOROTHIAZIDE:Urinary tract disorder:1.04), (CARFILZOMIB:Urinary tract disorder:0.26), (CARVEDILOL PHOSPHATE:Urinary tract disorder:0.88), (CIMETIDINE:Urinary tract disorder:0.19), (CLINDAMYCIN:Urinary tract disorder:0.08), (CLINDAMYCINCLINDAMYCIN PHOSPHATE:Urinary tract disorder:0.52), (CLOTRIMAZOLE:Urinary tract disorder:0.19), (COD LIVER OIL:Urinary tract disorder:0.07), (DACLIZUMAB:Urinary tract disorder:0.19), (DAPAGLIFLOZIN PROPANEDIOL:Urinary tract disorder:0.36), (DOXEPIN:Urinary tract disorder:0.05), (DOXEPIN HYDROCHLORIDE:Urinary tract disorder:0), (ELETRIPTAN HYDROBROMIDE:Urinary tract disorder:0.4), (EMPAGLIFLOZINLINAGLIPTIN:Urinary tract disorder:0.07), (EPINEPHRINE:Urinary tract disorder:0.17), (ESTROGENS, CONJUGATEDMEDROXYPROGESTERONE ACETATE:Urinary tract disorder:0.23), (ESZOPICLONE:Urinary tract disorder:0.69), (ETORICOXIB:Urinary tract disorder:0.13), (EVOLOCUMAB:Urinary tract disorder:1.33), (EXEMESTANE:Urinary tract disorder:0.28), (FLUTICASONE FUROATEVILANTEROL TRIFENATATE:Urinary tract disorder:0.25), (FULVESTRANT:Urinary tract disorder:0.74), (HALOPERIDOL:Urinary tract disorder:0.3), (HERBALSTURMERIC:Urinary tract disorder:0.26), (HYDROCHLOROTHIAZIDEIRBESARTAN:Urinary tract disorder:0.55), (HYDROMORPHONE:Urinary tract disorder:0.33), (IBRUTINIB:Urinary tract disorder:0.61), (IMATINIB MESYLATE:Urinary tract disorder:0.49), (INDAPAMIDE:Urinary tract disorder:0.55), (INDAPAMIDEPERINDOPRIL:Urinary tract disorder:0.68), (INFLUENZA VIRUS VACCINE:Urinary tract disorder:0.01), (LABETALOLLABETALOL HYDROCHLORIDE:Urinary tract disorder:0.39), (LACOSAMIDE:Urinary tract disorder:0.01), (LERCANIDIPINE HYDROCHLORIDE:Urinary tract disorder:0.63), (LEVALBUTEROL HYDROCHLORIDE:Urinary tract disorder:0.46), (LIDOCAINE HYDROCHLORIDE:Urinary tract disorder:0.21), (LITHIUM CARBONATE:Urinary tract disorder:0.59), (LOMITAPIDE MESYLATE:Urinary tract disorder:0.21), (MEGESTROL ACETATE:Urinary tract disorder:0.51), (METAXALONE:Urinary tract disorder:1.19), (METHIMAZOLE:Urinary tract disorder:0.16), (METHOCARBAMOL:Urinary tract disorder:0.22), (METRONIDAZOLE:Urinary tract disorder:0.93), (MOXIFLOXACIN HYDROCHLORIDE:Urinary tract disorder:0.85), (NALOXONE HYDROCHLORIDEOXYCODONE HYDROCHLORIDE:Urinary tract disorder:0.64), (NEBIVOLOL:Urinary tract disorder:0.63), (NILOTINIB:Urinary tract disorder:0.67), (OXCARBAZEPINE:Urinary tract disorder:1.42), (PACLITAXEL:Urinary tract disorder:0.31), (PANTOPRAZOLE MAGNESIUM:Urinary tract disorder:0.42), (PEGINTERFERON BETA-1A:Urinary tract disorder:0.16), (PEMETREXED DISODIUM:Urinary tract disorder:0.2), (PIOGLITAZONE HYDROCHLORIDE:Urinary tract disorder:0.11), (PRAZOSIN:Urinary tract disorder:0.08), (PROPAFENONE HYDROCHLORIDE:Urinary tract disorder:0.14), (RIZATRIPTAN BENZOATE:Urinary tract disorder:0.33), (ROFLUMILAST:Urinary tract disorder:0.11), (SCOPOLAMINE:Urinary tract disorder:0.55), (SECUKINUMAB:Urinary tract disorder:0.49), (TETRACYCLINE:Urinary tract disorder:0.51), (THYROID, PORCINE:Urinary tract disorder:0.45), (UMECLIDINIUM BROMIDEVILANTEROL TRIFENATATE:Urinary tract disorder:0.13), (VILAZODONE HYDROCHLORIDE:Urinary tract disorder:0.22), (.ALPHA.-TOCOPHEROLVITAMINS:Urinary tract infection:0.09), (ABOBOTULINUMTOXINA:Urinary tract infection:0.57), (ACAMPROSATE:Urinary tract infection:0.24), (ACECLOFENAC:Urinary tract infection:0.55), (ACETAMINOPHENMETHOCARBAMOL:Urinary tract infection:0.17), (ACETAMINOPHENOXYCODONEOXYCODONE HYDROCHLORIDE:Urinary tract infection:2.3), (ACETYLCARNITINE HYDROCHLORIDE:Urinary tract infection:0.14), (ACTIVATED CHARCOAL:Urinary tract infection:1.25), (AGALSIDASE BETA:Urinary tract infection:1.86), (AJMALINE:Urinary tract infection:0.01), (ALBUTEROLIPRATROPIUM BROMIDE:Urinary tract infection:0.06), (ALDESLEUKIN:Urinary tract infection:0.83), (ALUMINUM HYDROXIDEMAGNESIUM OXIDEOXETHAZAINE:Urinary tract infection:0.55), (AMBROXOLCLENBUTEROL:Urinary tract infection:0.15), (AMLODIPINEVALSARTAN:Urinary tract infection:1.61), (AMMONIUM CHLORIDEDEXTROMETHORPHAN\SODIUM CITRATE:Urinary tract infection:0.66), (AMOXICILLINCLARITHROMYCINESOMEPRAZOLE:Urinary tract infection:1.04), (ANAKINRA:Urinary tract infection:0.18), (ANHYDROUS CITRIC ACIDASPIRIN\SODIUM BICARBONATE:Urinary tract infection:0.26), (ARIPIPRAZOLE LAUROXIL:Urinary tract infection:0.43), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Urinary tract infection:0.35), (ASPIRINDIHYDROXYALUMINUM AMINOACETATE ANHYDROUSMAGNESIUM CARBONATE:Urinary tract infection:0.74), (ASUNAPREVIR:Urinary tract infection:0.41), (AVOCADO OILSOYBEAN OIL:Urinary tract infection:0.16), (AZELNIDIPINE:Urinary tract infection:0.59), (AZITHROMYCIN OR METHOTREXATE SODIUM:Urinary tract infection:0.77), (BALSALAZIDE DISODIUM:Urinary tract infection:1.24), (BAMIFYLLINE HYDROCHLORIDE:Urinary tract infection:0.32), (BEPRIDIL:Urinary tract infection:0.4), (BERAPROST:Urinary tract infection:0.19), (BETAHISTINE MESILATE:Urinary tract infection:0.23), (BETAXOLOL HYDROCHLORIDE:Urinary tract infection:0.64), (BLACK COHOSH:Urinary tract infection:0.17), (BLINATUMOMAB:Urinary tract infection:0.9), (BOCEPREVIR:Urinary tract infection:0.42), (BOCOCIZUMAB:Urinary tract infection:0.31), (BOSUTINIB:Urinary tract infection:0.37), (BRINZOLAMIDETIMOLOL MALEATE:Urinary tract infection:0.09), (BRIVARACETAM:Urinary tract infection:0.25), (BROMHEXINE:Urinary tract infection:0.22), (BUCILLAMINE:Urinary tract infection:0.2), (BUPRENORPHINENALOXONE:Urinary tract infection:0.19), (CABAZITAXEL:Urinary tract infection:0.3), (CABOZANTINIB:Urinary tract infection:0.41), (CABOZANTINIB S-MALATE:Urinary tract infection:0.49), (CALCIUM ACETATEMAGNESIUM ACETATE\POTASSIUM ACETATE\SODIUM ACETATE\SODIUM CHLORIDE:Urinary tract infection:0.05), (CALCIUM CARBONATECHOLECALCIFEROLMAGNESIUM CARBONATE:Urinary tract infection:0.64), (CALCIUM\MAGNESIUM\ZINC:Urinary tract infection:0.97), (CALCIUMVITAMINS:Urinary tract infection:0.03), (CAMPHORDIPHENHYDRAMINE\ZINC OXIDE:Urinary tract infection:0.31), (CANNABIDIOL:Urinary tract infection:0.2), (CARBASALATE:Urinary tract infection:0.12), (CARMUSTINE:Urinary tract infection:0.33), (CARTEOLOL HYDROCHLORIDE:Urinary tract infection:0.23), (CARVEDILOL PHOSPHATE:Urinary tract infection:2.75), (CEFOTAXIMECEFOTAXIME SODIUM:Urinary tract infection:1.28), (CHLORDIAZEPOXIDE:Urinary tract infection:0.32), (CHLORMADINONE ACETATE:Urinary tract infection:0.53), (CHLOROQUINE PHOSPHATE:Urinary tract infection:0.35), (CHLORPROTHIXENE:Urinary tract infection:0.21), (CHOLESTEROL:Urinary tract infection:0.32), (CICLOPIROX OLAMINE:Urinary tract infection:0.29), (CIPROFIBRATE:Urinary tract infection:0.98), (CISAPRIDE:Urinary tract infection:0.33), (CISATRACURIUM:Urinary tract infection:0.68), (CLIKSTAR:Urinary tract infection:0.38), (CLODRONATE DISODIUM:Urinary tract infection:0.13), (CLOFAZIMINE:Urinary tract infection:0.02), (CLOXACILLIN SODIUM:Urinary tract infection:0.44), (CLOXAZOLAM:Urinary tract infection:0.46), (COBICISTATELVITEGRAVIREMTRICITABINETENOFOVIR ALAFENAMIDE FUMARATE:Urinary tract infection:0.08), (CYPROTERONE:Urinary tract infection:0.09), (CYPROTERONE ACETATE:Urinary tract infection:0.65), (DEFERIPRONE:Urinary tract infection:0.89), (DESFLURANE:Urinary tract infection:0.4), (DESVENLAFAXINE:Urinary tract infection:0.47), (DEXPANTHENOL:Urinary tract infection:0.16), (DEXTROAMPHETAMINE:Urinary tract infection:0.59), (DISOPYRAMIDE:Urinary tract infection:0.06), (EDARAVONE:Urinary tract infection:0.38), (EFINACONAZOLE:Urinary tract infection:0.41), (ELTROMBOPAG OLAMINE:Urinary tract infection:0.73), (EMPAGLIFLOZINLINAGLIPTIN:Urinary tract infection:1.52), (EPINASTINE HYDROCHLORIDE:Urinary tract infection:1.4), (EPIRUBICIN HYDROCHLORIDE:Urinary tract infection:0.32), (ERIBULIN:Urinary tract infection:0.95), (ERIBULIN MESYLATE:Urinary tract infection:0.4), (ERYTHROMYCINSULFISOXAZOLE:Urinary tract infection:0.03), (ESMOLOL HYDROCHLORIDE:Urinary tract infection:1.05), (ESTROGENS, ESTERIFIEDMETHYLTESTOSTERONE:Urinary tract infection:0.03), (ETHACRYNIC ACID:Urinary tract infection:0.27), (ETRETINATE:Urinary tract infection:1.21), (EUCALYPTOLMENTHOLMETHYL SALICYLATETHYMOL:Urinary tract infection:0.04), (FERROUS BISGLYCINATE:Urinary tract infection:0.49), (FERROUS GLYCINE SULFATE:Urinary tract infection:0.65), (FERROUS SULFATEGLYCINE:Urinary tract infection:1.39), (FLUNARIZINE HYDROCHLORIDE:Urinary tract infection:0.37), (FLUOCINOLONE ACETONIDE:Urinary tract infection:1.39), (FLUOROURACILLEUCOVORIN CALCIUMOXALIPLATIN:Urinary tract infection:0.27), (FLUPENTIXOL:Urinary tract infection:0.05), (FLUPHENAZINE:Urinary tract infection:0.34), (FLURAZEPAM HYDROCHLORIDE:Urinary tract infection:0.17), (FROVATRIPTAN SUCCINATE:Urinary tract infection:0.15), (FUROSEMIDESPIRONOLACTONE:Urinary tract infection:0.26), (GADOBENATE DIMEGLUMINE:Urinary tract infection:1.03), (GADOXETATE DISODIUM:Urinary tract infection:0.18), (GLIPIZIDEMETFORMIN HYDROCHLORIDE:Urinary tract infection:0.56), (GLYCERINHYPROMELLOSESPOLYETHYLENE GLYCOL 400:Urinary tract infection:0.16), (GUANFACINE HYDROCHLORIDE:Urinary tract infection:0.79), (HYDROCODONE BITARTRATEIBUPROFEN:Urinary tract infection:1.04), (HYDROCORTISONE HEMISUCCINATE:Urinary tract infection:0.01), (HYDROXYCHLOROQUINE DIPHOSPHATE:Urinary tract infection:1.11), (IBRITUMOMAB TIUXETAN:Urinary tract infection:0.3), (IDARUCIZUMAB:Urinary tract infection:0.55), (ILOPROST:Urinary tract infection:1.59), (IMIGLUCERASE:Urinary tract infection:1.18), (IMMUNE GLOBULIN NOS:Urinary tract infection:0.81), (INFLIXIMAB-DYYB:Urinary tract infection:1.02), (INOTUZUMAB OZOGAMICIN:Urinary tract infection:0.68), (INSULIN PORKINSULIN PURIFIED PORK:Urinary tract infection:0.49), (INTERFERON:Urinary tract infection:1.77), (INTERFERON ALFA-2A:Urinary tract infection:1.79), (INTERFERON ALFA-2A OR INTERFERON ALFA-2B:Urinary tract infection:0.3), (INTERFERON ALFA-2B:Urinary tract infection:1.24), (INTERFERON GAMMA-1B:Urinary tract infection:1.32), (IOPROMIDE:Urinary tract infection:0.26), (IRON POLYMALTOSE:Urinary tract infection:0.32), (ISONIAZIDPYRAZINAMIDERIFAMPIN:Urinary tract infection:0.48), (ISONIAZIDRIFAMPIN:Urinary tract infection:0.41), (IVABRADINE HYDROCHLORIDE:Urinary tract infection:0.32), (LABETALOL HYDROCHLORIDE:Urinary tract infection:2.57), (LAFUTIDINE:Urinary tract infection:0.66), (LANREOTIDE:Urinary tract infection:0.62), (LAPATINIB:Urinary tract infection:0.21), (LAPATINIB DITOSYLATE:Urinary tract infection:1.06), (LEVOMEFOLATE CALCIUM:Urinary tract infection:0.7), (LEVOSIMENDAN:Urinary tract infection:0.2), (LOMITAPIDE MESYLATE:Urinary tract infection:0.96), (LOMUSTINE:Urinary tract infection:0.44), (LOXOPROFEN SODIUM DIHYDRATE:Urinary tract infection:0.01), (LUTETIUM LU-177:Urinary tract infection:0.36), (MAGNESIUM ASPARTATE\POTASSIUM ASPARTATE:Urinary tract infection:0.62), (MAGNESIUM PIDOLATE:Urinary tract infection:0.31), (MEPOLIZUMAB:Urinary tract infection:1.16), (MESTRANOLNORETHINDRONE:Urinary tract infection:0.07), (METFORMIN HYDROCHLORIDEROSIGLITAZONE MALEATE:Urinary tract infection:0.03), (METFORMIN HYDROCHLORIDESAXAGLIPTIN:Urinary tract infection:0.5), (METHYLENE BLUE:Urinary tract infection:0.46), (MIGLUSTAT:Urinary tract infection:0.31), (MOEXIPRIL HYDROCHLORIDE:Urinary tract infection:1.36), (MORPHINE SULFATE\NALTREXONE HYDROCHLORIDE:Urinary tract infection:1.05), (MORPHINE\MORPHINE HYDROCHLORIDE\MORPHINE SULFATE\MORPHINE TARTRATE:Urinary tract infection:0.14), (NEFAZODONE HYDROCHLORIDE:Urinary tract infection:0.03), (NEOMYCIN:Urinary tract infection:0.49), (NUSINERSEN:Urinary tract infection:0.44), (OLANZAPINE PAMOATE:Urinary tract infection:0.02), (OLOPATADINE:Urinary tract infection:1.05), (OXYBUTYNINOXYBUTYNIN CHLORIDE:Urinary tract infection:0.68), (PARATHYROID HORMONE:Urinary tract infection:0.16), (PEGINTERFERON ALFA-2B:Urinary tract infection:0.57), (PEGVISOMANT:Urinary tract infection:0.55), (PENICILLAMINE:Urinary tract infection:0.01), (PERAMPANEL:Urinary tract infection:0.42), (PERINDOPRIL ARGININE:Urinary tract infection:0.09), (PERPHENAZINE:Urinary tract infection:0.3), (PHENOBARBITAL SODIUM:Urinary tract infection:0.48), (PIMECROLIMUS:Urinary tract infection:0.89), (PIRACETAM:Urinary tract infection:0.21), (PIRBUTEROL ACETATE:Urinary tract infection:0.06), (PORFIMER SODIUM:Urinary tract infection:0.68), (POTASSIUM IODIDE:Urinary tract infection:0.54), (POTASSIUM PHOSPHATE, MONOBASIC:Urinary tract infection:0.14), (PRAMOXINE HYDROCHLORIDE:Urinary tract infection:0.23), (PRASUGREL:Urinary tract infection:0.1), (PREDNISOLONE SODIUM PHOSPHATE:Urinary tract infection:1), (PYRIDOXAL:Urinary tract infection:0), (PYRIDOXAL PHOSPHATE:Urinary tract infection:0.52), (PYRIMETHAMINE:Urinary tract infection:0.59), (QUINACRINE:Urinary tract infection:0.59), (REMIFENTANIL:Urinary tract infection:0.52), (REMIFENTANIL HYDROCHLORIDE:Urinary tract infection:0.34), (RETINOL:Urinary tract infection:1.88), (RILPIVIRINE HYDROCHLORIDE:Urinary tract infection:0.32), (ROPIVACAINE:Urinary tract infection:0.15), (RUCAPARIB CAMSYLATE:Urinary tract infection:0.1), (RUPATADINE FUMARATE:Urinary tract infection:0.18), (SARPOGRELATE:Urinary tract infection:1.23), (SCOPOLAMINE HYDROBROMIDE:Urinary tract infection:0.19), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Urinary tract infection:0.26), (SPIRAMYCIN:Urinary tract infection:0.89), (SUCCINYLCHOLINE CHLORIDE:Urinary tract infection:0.97), (SUPLATAST TOSILATE:Urinary tract infection:0.51), (TAFAMIDIS MEGLUMINE:Urinary tract infection:0.24), (TAMOXIFEN CITRATE:Urinary tract infection:1.17), (TEDUGLUTIDE\WATER:Urinary tract infection:1.36), (TELOTRISTAT ETHYL:Urinary tract infection:0.6), (THIAMYLAL SODIUM:Urinary tract infection:0.18), (THIOPENTAL:Urinary tract infection:0.67), (THROMBOMODULIN ALFA:Urinary tract infection:0.05), (TIBOLONE:Urinary tract infection:0.46), (TIMOLOLTRAVOPROST:Urinary tract infection:0.21), (TIPIRACIL HYDROCHLORIDETRIFLURIDINE:Urinary tract infection:0.77), (TRABECTEDIN:Urinary tract infection:0.14), (TREPROSTINIL DIOLAMINE:Urinary tract infection:0.02), (TRIMETAZIDINE:Urinary tract infection:0.01), (UMECLIDINIUM BROMIDE:Urinary tract infection:1.14), (VALDECOXIB:Urinary tract infection:0.53), (VARDENAFIL HYDROCHLORIDE TRIHYDRATE:Urinary tract infection:0.7), (VECURONIUM BROMIDE:Urinary tract infection:0.88), (YEAST:Urinary tract infection:1.1), (ZICONOTIDE ACETATE:Urinary tract infection:0.65), (ZILEUTON:Urinary tract infection:0.75), (ZINC SULFATE\ZINC SULFATE ANHYDROUS:Urinary tract infection:0.28), (ZIPRASIDONE HYDROCHLORIDEZIPRASIDONE MESYLATE:Urinary tract infection:0.75), (ZIV-AFLIBERCEPT:Urinary tract infection:0.57), (.ALPHA.1-PROTEINASE INHIBITOR HUMAN:Urticaria:1.01), (ACENOCOUMAROL:Urticaria:0.05), (ACETAMINOPHENOXYCO DONEOXYCODONE HYDROCHLORIDE:Urticaria:0.39), (ADO-TRASTUZUMAB EMTANSINE:Urticaria:0.17), (AGALSIDASE BETA:Urticaria:0.04), (ALBIGLUTIDE:Urticaria:0.05), (ALENDRONIC ACID:Urticaria:0.41), (AMANTADINE:Urticaria:0.38), (BIMATOPROST:Urticaria:0.48), (BISA CODYL OR DOCUSATE SODIUM:Urticaria:1.08), (CALCITONIN SALMON:Urticaria:0.45), (CALCIUM ACETATE:Urticaria:0.42), (CALCIUM\MAGNESIUM:Urticaria:0.53), (CANAGLIFLOZINM ETFORMIN HYDROCHLORIDE:Urticaria:0.57), (CAPTOPRIL:Urticaria:0.46), (CARVEDILOL PHOSPHATE:Urticaria:1.05), (CEFOTAXIME SODIUM:Urticaria:0.22), (CHLORPHENIRAMINEHYDROCODONE:Urticaria:0.12), (CHON DROITIN SULFATE (BOVINE)GLUCOSAMINE HYDROCHLORIDE:Urticaria:0.29), (CILASTATIN SODIUMIMIPENEM:Urticaria:0.04), (CLINDAMYCINCLINDAMYCIN PHOSPHATE:Urticaria:1.64), (CLOMIPRAMINE HYDROCHLORIDE:Urticaria:0.11), (CLOZAPINE:Urticaria:0.93), (COD LIVER OIL:Urticaria:0.1), (CORTICOTROPIN:Urticaria:0.33), (DACLIZUMAB:Urticaria:1.1), (DASA TINIB:Urticaria:1.37), (DOFETILIDE:Urticaria:0.4), (DORZOLAMIDE:Urticaria:0.19), (DRON ABINOL:Urticaria:0.51), (ECONAZOLE:Urticaria:0.36), (ELOTUZUMAB:Urticaria:0.09), (EM PAGLIFLOZINLINAGLIPTIN:Urticaria:0.57), (EPLERENONE:Urticaria:0.31), (ESTROGENS, CONJUGATEDMEDROXYPROGESTERONE ACETATE:Urticaria:0.61), (ETORICOXIB:Urticaria:0.48), (ETRETINATE:Urticaria:0.42), (FEN OFIBRIC ACID:Urticaria:0.26), (FINGOLIMOD HYDROCHLORIDE:Urticaria:0.86), (FLUDROCORTISONE ACETATE:Urticaria:0.21), (FORMOTEROL:Urticaria:0.12), (GIMERACILOTERACILTEGA FUR:Urticaria:0.2), (GLYCOPYRROLATE:Urticaria:0.15), (GLYCOPYRRONIUM:Urticaria:0.2), (HYDROCHLOROTHIAZIDEIRBESARTAN:Urticaria:0.73), (HYDROXYCHLOROQUIN E DIPHOSPHATE:Urticaria:0.75), (ICOSAPENT ETHYL:Urticaria:0.06), (ILOPROST:Urticaria:0.26), (INDACATEROL:Urticaria:0.23), (INDAP AMIDEPERINDOPRIL:Urticaria:0.66), (INFLIXIMAB-DYYB:Urticaria:0.91), (IRINOTECAN HYDROCHLORIDE:Urticaria:0.41), (IRON SUCROSE:Urticaria:0.18), (KETOROLACKETOROLAC TROMETHAMINE:Urticaria:0.1), (LABETALOLLABETALOL HYDROCHLORIDE:Urticaria:1.05), (LACTOBACILLUS RHAMNOSUS:Urticaria:0.22), (LAPATINIB DITOSYLATE:Urticaria:0.09), (LERCANIDIPINE HYDROCHLORIDE:Urticaria:0.65), (LIOTHYRONINE:Urticaria:0.1), (MEDROXYPROGEST ERONE ACETATE:Urticaria:0.54), (MEFENAMIC ACID:Urticaria:0.06), (MEGESTROL ACETATE:Urticaria:0.1), (MEPOLIZUMAB:Urticaria:0.6), (METHENOLONE ENANTHATE:Urticaria:0.03), (MIRABEGRON:Urticaria:0.39), (MORPHINE SULFATE\NALTREXONE HYDROCHLORIDE:Urticaria:0.08), (MOXIFLOXACIN:Urticaria:0.25), (NATEGLINIDE:Urtic aria:0.14), (NIRAPARIB:Urticaria:0.04), (NORTRIPTYLINE HYDROCHLORIDE:Urticaria:0.13), (OLODATEROL HYDROCHLORIDETIOTROPIUM BROMIDE MONOHYDRATE:Urticaria:1.19), (OXYBUTYNIN CHLORIDE:Urticaria:0.71), (PANTOPRAZOLE MAGNESIUM:Urticaria:1.83), (PAZOPANIB HYDROCHLORIDE:Urticaria:0.69), (PEGINTERFERON BETA-1A:Urticaria:0.68), (PEMETREXED DISODIUM:Urticaria:1.09), (PENICILLIN V:Urticaria:0.1), (PHENOBARBITAL:Urticaria:0.72), (PHENYTOIN SODIUM:Urticaria:0.28), (PHYTONADIONE:Urticaria:0.43), (PIROXICAM:Urticaria:0.47), (P RAZOSIN HYDROCHLORIDE:Urticaria:0.03), (PRIMIDONE:Urticaria:1.27), (REGORAFENIB:Urticaria: 0.44), (ROFLUMILAST:Urticaria:0.96), (ROPINIROLE:Urticaria:0.35), (SACUBITRILVALSA RTAN:Urticaria:0.68), (SODIUM FERROUS CITRATE:Urticaria:0.18), (SULFAMETHOXAZOLE:Urticaria:0.92), (SUMATRIPTANSUMA TRIPTAN SUCCINATE:Urticaria:1.42), (TESTOSTERONE UNDECANOATE:Urticaria:0.13), (TETRACYCLINE:Urticaria:1), (TIMOLOL:Urticaria:0.61), (TINZAPARIN SODIUM:Urticaria:0.48), (TIPIRACIL HYDROCHLORIDETRIFLURIDINE:Urticaria:0.06), (UMECLIDINIUM BROMIDE:Urticaria:0.26), (VANCOMYCIN HYDROCHLORIDE:Urticaria:0.38), (VITAMIN A:Urticaria:0.51), (ZILEUTON:Urticaria:0.23), (CELECOXIB:Uterine cancer:0.19), (DEXLANSOPRAZOLE:Uterine cancer:0.12), (LENALIDOMIDE:Uterine cancer:0.19), (RISEDRONATE SODIUM:Uterine cancer:0.28), (SPIRONOLACTONE:Uterine cancer:0.13), (ALLOPURINOL:Uterine leiomyoma:0.15), (ANASTROZOLE:Uterine leiomyoma:0.56), (CERTOLIZUMAB PEGOL:Uterine leiomyoma:0.63), (CHOLESTYRAMINE:Uterine leiomyoma:0.07), (DASATINIB:Uterine leiomyoma:0.11), (DIENOGEST:Uterine leiomyoma:0.04), (DIGOXIN:Uterine leiomyoma:0.01), (DILTIAZEM HYDROCHLORIDE:Uterine leiomyoma:0.36), (EPINEPHRINE:Uterine leiomyoma:0.01), (ESZOPICLONE:Uterine leiomyoma:0.26), (EVOLOCUMAB:Uterine leiomyoma:0.42), (HYDROXYZINE PAMOATE:Uterine leiomyoma:0.22), (IBRUTINIB:Uterine leiomyoma:0.06), (LISDEXAMFETAMINE DIMESYLATE:Uterine leiomyoma:0.46), (METHYLPREDNISOLONE ACETATE:Uterine leiomyoma:0.1), (METHYLPREDNISOLONE SODIUM SUCCINATE:Uterine leiomyoma:0.31), (PAROXETINE:Uterine leiomyoma:0.39), (PENICILLIN:Uterine leiomyoma:0.11), (PRAVASTATIN:Uterine leiomyoma:0.18), (PRIMIDONE:Uterine leiomyoma:0.22), (RISEDRONATE SODIUM:Uterine leiomyoma:0.12), (SACUBITRILVALSARTAN:Uterine leiomyoma:0), (SECUKINUMAB:Uterine leiomyoma:0.1), (TERIFLUNOMIDE:Uterine leiomyoma:0.63), (TORSEMIDE:Uterine leiomyoma:0.11), (ZOLPIDEMZOLPIDEM TARTRATE:Uterine leiomyoma:0.41), (ACETAMINOPHENOXYCODONE HYDROCHLORIDE:Uveitis:0.13), (AMIODARONE:Uveitis:0.43), (AMIODARONE HYDROCHLORIDE:Uveitis:0.12), (ARMODAFINIL:Uveitis:0.31), (BUMETANIDE:Uveitis:0.17), (CARTEOLOL HYDROCHLORIDE:Uveitis:0.07), (CLOTRIMAZOLE:Uveitis:0.03), (DACLIZUMAB:Uveitis: 0.16), (DIVALPROEX SODIUM:Uveitis:0.21), (ENOXAPARIN:Uveitis:0.24), (ENOXAPARIN SODIUM:Uveitis:0.04), (EVOLOCUMAB:Uveitis:1.08), (HYDROXYCHLOROQUINE DIPHOSPHATE:Uveitis:0.19), (INDAPAMIDE:Uveitis:0), (LISDEXAMFETAMINE DIMESYLATE:Uveitis:0.04), (METHYLPREDNISOLONE ACETATE:Uveitis:0.17), (METRONIDAZOLE:Uveitis:1.08), (OXCARBAZEPINE:Uveitis:0.09), (OXYGEN:Uveitis:0.08), (PANTOPRAZOLE MAGNESIUM:Uveitis:0.16), (POLYETHYLENE GLYCOLS:Uveitis:0.15), (PRAMIPEXOLE DIHYDROCHLORIDE:Uveitis:0.17), (PRAVASTATIN:Uveitis:0.58), (PROGESTERONE:Uvei tis:0.15), (RABEPRAZOLE SODIUM:Uveitis:0.37), (RALOXIFENE HYDROCHLORIDE:Uveitis:0.03), (RANOLAZINE:Uveitis:0.16), (SACUBITRILVALSARTA N:Uveitis:0.16), (SODIUM OXYBATE:Uveitis:0.44), (TIZANIDINE HYDROCHLORIDE:Uveitis:0.24), (TORSEMIDE:Uveitis:0.24), (NAPROXEN:Vaginal haemorrhage:0.23), (BUPROPION HYDROCHLORIDE:Vaginal infection:0.38), (CALCIUM CARBONATECHOLECALCIFEROL:Vaginal infection:0.26), (IBRUTINIB:Vaginal infection:0.22), (IRON:Vaginal infection:0.27), (METHYLPREDNISOLONE SODIUM SUCCINATE:Vaginal infection:0.04), (RAMIPRIL:Vaginal infection:0.01), (TAMSULOSIN:Vaginal infection:0.02), (TRIAMCINOLONE ACETONIDE:Vaginal infection:0.16), (WARFARIN:Vaginal infection:0.16), (IBUPROFEN:Varices oesophageal:0.11), (EVOLOCUMAB:Varicose vein:0.17), (LOVASTATIN:Varicose vein:0.03), (NAPROXEN:Varicose vein:0.02), (DEXLANSOPRAZOLE:Vascular graft:0.3), (DICLOFENAC:Vascular graft:0.14), (DULOXETINE HYDROCHLORIDE:Vascular graft:0.05), (ENALAPRIL:Vascular graft:0.85), (IBRUTINIB:Vascular graft:0.3), (RANOLAZINE:Vascular graft:0.28), (BUMETANIDE:Vasculitis:1.24), (CARFILZOMIB:Vasculitis:0.14), (CHOLESTYR AMINE:Vasculitis:0.22), (DACLIZUMAB:Vasculitis:0.03), (DASATINIB:Vasculitis:0.45), (DIC LOFENAC:Vasculitis:0.9), (DICYCLOMINE HYDROCHLORIDE:Vasculitis:0.02), (DIGOXIN:Vasculitis:0.82), (DIVALPROEX SODIUM:Vasculitis:0.43), (ELETRIPTAN HYDROBROMIDE:Vasculitis:0.17), (ENALAPRIL MALEATE:Vasculitis:0.33), (ESZOPICLONE:Vasculitis:0.5), (EVOLOCUMAB:Vasculitis:0.61), (EZETIMIBESIMVASTATIN:Vasculitis:0.08), (FENOFIBRATE:Vasculitis:0.33), (FLUTICA SONE:Vasculitis:0.05), (FLUTICASONE FUROATEVILANTEROL TRIFENATATE:Vasculitis:0.08), (HYDROCODONE:Vasculitis:0.25), (HYDROXYCHLOROQ UINE DIPHOSPHATE:Vasculitis:0.26), (HYDROXYZINE PAMOATE:Vasculitis:0.14), (IBRUTINIB:Vasculitis:1.44), (ISOSORBIDE MONONITRATE:Vasculitis:0.02), (LERCANIDIPINE:Vasculitis:0.1), (NAPROXEN SODIUM:Vasculitis:0.24), (NILOTINIB:Vasculitis:0.54), (NITROFURANTOIN:Vasculitis:0.01), (NYSTATIN:Vasculitis:0.42), (OMEGA-3-ACID ETHYL ESTERS:Vasculitis:0.04), (OXYGEN:Vasculitis:0.44), (PAROXETINEPAROXETINE HYDROCHLORIDE:Vasculitis:0.07), (POLYETHYLENE GLYCOL 3350:Vasculitis:0.01), (POLYETHYLENE GLYCOLS:Vasculitis:0.48), (PRAVASTATIN SODIUM:Vasculitis:0.93), (PROMETHAZINEPROMETHAZINE HYDROCHLORIDE:Vasculitis:0.16), (RANOLAZINE:Vasculitis:0.39), (SACUBITRILVALS ARTAN:Vasculitis:0.6), (SODIUM OXYBATE:Vasculitis:0.16), (SOTALOL:Vasculitis:0.14), (TEMAZEPAM:Vasculitis:0.14), (TH ALIDOMIDE:Vasculitis:0.28), (TIZANIDINE HYDROCHLORIDE:Vasculitis:0.15), (UMECLIDINIUM BROMIDEVILANTEROL TRIFENATATE:Vasculitis:0.07), (VANCOMYCIN HYDROCHLORIDE:Vasculitis:0.01), (ZOLPIDEMZOLPIDEM TARTRATE:Vasculitis:0.64), (ZONISAMIDE:Vasculitis:0.2), (EVOLOCUMAB:Vasodilatation: 0.29), (IBRUTINIB:Vasodilatation:0.67), (NEBIVOLOL:Vasodilatation:0.15), (PANTOPRAZOL E:Vasodilatation:0.17), (ISOFLURANE:Vasopressive therapy:0.29), (NEOSTIGMINE:Vasopressive therapy:0.1), (ROPIVACAINE:Vasopressive therapy:0.01), (AMIODARONE HYDROCHLORIDE:Venous thrombosis:0.57), (BUMETANIDE:Venous thrombosis:0.62), (BUPROPION HYDROCHLORIDE:Venous thrombosis:0.43), (CALCITRIOL:Venous thrombosis:0.12), (CANDESARTAN:Venous thrombosis:0.67), (CANDESARTAN CILEXETILHYDROCHLOROTHIAZIDE:Venous thrombosis:0.36), (CETIRIZINE HYDROCHLORIDE:Venous thrombosis:0.1), (CETUXIMAB:Venous thrombosis:0.58), (CLOZAPINE:Venous thrombosis:0.54), (CORTISONEHYDROCORTISONE:Venous thrombosis:0.14), (DEXLANSOPRAZOLE:Venous thrombosis:0.43), (DILTIAZEM HYDROCHLORIDE:Venous thrombosis:1.14), (DIPHENHYDRAMINE:Venous thrombosis:0), (EPIRUBICIN:Venous thrombosis:0.69), (ERGOCALCIFEROL:Venous thrombosis:0.06), (EVOLOCUMAB:Venous thrombosis:0.36), (FERROUS SULFATEFERROUS SULFATE, DRIED:Venous thrombosis:0.74), (FINGOLIMOD HYDROCHLORIDE:Venous thrombosis:0.34), (FISH OIL:Venous thrombosis:0.32), (FLUCONAZOLE:Venous thrombosis:0.78), (HYDROCHLOROTHIAZIDELISINOPRIL:Venous thrombosis:0.15), (IBRUTINIB:Venous thrombosis:1.16), (IMATINIB MESYLATE:Venous thrombosis:0.72), (INSULIN HUMAN:Venous thrombosis:0.14), (IRINOTECAN:Venous thrombosis:0.67), (LERCANIDIPINE HYDROCHLORIDE:Venous thrombosis:0.29), (LITHIUM CARBONATE:Venous thrombosis:0.16), (LOVASTATIN:Venous thrombosis:0.41), (MELOXICAM:Venous thrombosis:0.15), (NILOTINIB:Venous thrombosis:1.25), (NITROFURANTOIN:Venous thrombosis:0.04), (PAZOPANIB HYDROCHLORIDE:Venous thrombosis:0.53), (POTASSIUM:Venous thrombosis:0.33), (PRAVASTATIN SODIUM:Venous thrombosis:0.48), (PYRAZINAMIDE:Venous thrombosis:0.67), (SACUBITRILVALSARTAN:Venous thrombosis:0.66), (SECUKINUMAB:Venous thrombosis:0.42), (TAMSULOSIN:Venous thrombosis:0.91), (THALIDOMIDE:Venous thrombosis:0.68), (TOCILIZUMAB:Venous thrombosis:0.35), (ACETAMINOPHENCODEINE PHOSPHATE:Ventricular extrasystoles:0.51), (CALCIUM CARBONATECHOLECALCIFEROL:Ventricular extrasystoles:0.58), (CANDESARTAN CILEXETILHYDROCHLOROTHIAZIDE:Ventricular extrasystoles:0.19), (CEPHALEXIN:Ventricular extrasystoles:0.01), (CHOLESTYRAMINE:Ventricular extrasystoles:0.23), (CITALOPRAM HYDROBROMIDE:Ventricular extrasystoles:0.47), (DABIGATRAN:Ventricular extrasystoles:0.11), (DASATINIB:Ventricular extrasystoles:0.19), (DEVICE:Ventricular extrasystoles:0.1), (DOCUSATE SODIUM:Ventricular extrasystoles:0.59), (ENOXAPARIN SODIUM:Ventricular extrasystoles:0.23), (ERYTHROPOIETIN:Ventricular extrasystoles:0.04), (ESZOPICLONE:Ventricular extrasystoles:0.13), (EVOLOCUMAB:Ventricular extrasystoles:0.8), (FLUTICASONE:Ventricular extrasystoles:0.03), (GLYBURIDE:Ventricular extrasystoles:0.03), (ILOPROST:Ventricular extrasystoles:0.22), (LEDIPASVIRSOFOSBUVIR:Ventricular extrasystoles:0.09), (METHYLPREDNISOLONE SODIUM SUCCINATE:Ventricular extrasystoles:0.61), (METRONIDAZOLE:Ventricular extrasystoles:0.83), (NAPROXEN SODIUM:Ventricular extrasystoles:0.08), (NILOTINIB:Ventricular extrasystoles:0.06), (POLYETHYLENE GLYCOLS:Ventricular extrasystoles:0.06), (RALOXIFENE HYDROCHLORIDE:Ventricular extrasystoles:0.04), (TERIFLUNOMIDE:Ventricular extrasystoles:0.64), (THALIDOMIDE:Ventricular extrasystoles:0.07), (TOCILIZUMAB:Ventricular extrasystoles:0.2), (ACETAMINOPHENCODEINE PHOSPHATE:Ventricular tachycardia:0.44), (BORTEZOMIB:Ventricular tachycardia:0.07), (BROMAZEPAM:Ventricular tachycardia:0.02), (CALCITRIOL:Ventricular tachycardia:0.07), (CALCIUM ACETATE:Ventricular tachycardia:0.36), (CANDESARTAN CILEXETILHYDROCHLOROTHIAZIDE:Ventricular tachycardia:0.41), (CARFILZOMIB:Ventricular tachycardia:0), (CERTOLIZUMAB PEGOL:Ventricular tachycardia:0.03), (CLONIDINE:Ventricular tachycardia:0.03), (CLOTRIMAZOLE:Ventricular tachycardia:0.02), (DASATINIB:Ventricular tachycardia:0.17), (DEXLANSOPRAZOLE:Ventricular tachycardia:0.75), (DICLOFENAC SODIUM:Ventricular tachycardia:0.4), (DIVALPROEX SODIUM:Ventricular tachycardia:0.23), (ENALAPRIL MALEATE:Ventricular tachycardia:0.15), (ERYTHROPOIETIN:Ventricular tachycardia:0.28), (EVEROLIMUS:Ventricular tachycardia:0.23), (EVOLOCUMAB:Ventricular tachycardia:0.46), (FLUTICASONE:Ventricular tachycardia:0.07), (FLUTICASONE FUROATEVILANTEROL TRIFENATATE:Ventricular tachycardia:0.49), (ILOPROST:Ventricular tachycardia:0.72), (INDAPAMIDEPERINDOPRIL:Ventricular tachycardia:0.03), (INSULIN NOS:Ventricular tachycardia:0.17), (IPRATROPIUM:Ventricular tachycardia:0.15), (IRBESARTAN:Ventricular tachycardia:0.18), (LEDIPASVIRSOFOSBUVIR:Ventricular tachycardia:0.23), (LERCANIDIPINE HYDROCHLORIDE:Ventricular tachycardia:0.03), (LOVASTATIN:Ventricular tachycardia:0.33), (METFORMIN HYDROCHLORIDEROSIGLITAZONE MALEATE:Ventricular tachycardia:0.04), (METHYLPREDNISOLONE SODIUM SUCCINATE:Ventricular tachycardia:0.35), (NEBIVOLOL:Ventricular tachycardia:0.71), (NIFEDIPINE:Ventricular tachycardia:0.54), (NILOTINIB:Ventricular tachycardia:0.28), (OMEGA-3-ACID ETHYL ESTERS:Ventricular tachycardia:0.28), (OXCARBAZEPINE:Ventricular tachycardia:0.21), (PAROXETINEPAROXETINE HYDROCHLORIDE:Ventriculartachycardia:0.04), (PRAVASTATIN:Ventricular tachycardia:0.61), (PRIMIDONE:Ventricular tachycardia:0.03), (SECUKINUMAB:Ventricular tachycardia:0.02), (TERIFLUNOMIDE:Ventricular tachycardia:0.14), (TOFACITINIB CITRATE:Ventricular tachycardia:0.04), (.ALPHA.1-PROTEINASE INHIBITOR HUMAN:Vertigo:0.45), (ABIRATERONE ACETATE:Vertigo:0.33), (ACETAMINOPHENPROPOXYPHENE HYDROCHLORIDE:Vertigo:0.25), (ACETAMINOPHENTRAMADOL:Vertigo:0.1), (ADO-TRASTUZUMAB EMTANSINE:Vertigo:0.54), (AGALSIDASE BETA:Vertigo:0.16), (ALBIGLUTIDE:Vertigo:0.25), (ALCOHOL:Vertigo:0.27), (ALENDRONI C ACID:Vertigo:0.5), (ALFACALCIDOL:Vertigo:0.34), (ALFUZOSIN HYDROCHLORIDE:Vertigo:0.1), (ALISKIREN HEMIFUMARATE:Vertigo:0.04), (AMILORIDE HYDROCHLORIDEHYDROCHLOROTHIAZIDE:Vertigo:0.09), (AMISULPRIDE:Vertigo:0.07), (AMLODIPINE BESYLATEHYDROCHLOROTHIAZIDEOLMESARTAN MEDOXOMIL:Vertigo:0.1), (AMLODIPINEVALSARTAN:Vertigo:0.1), (ANTIHISTAMINES NOS:Vertigo:0.03), (BENAZEPRILBENAZEPRIL HYDROCHLORIDE:Vertigo:0.43), (BUPRENORPHINE HYDROCHLORIDE:Vertigo:0.66), (BUTYLSCOPOLAMINE BROMIDE:Vertigo:0.78), (CALCIPOTRIENE:Vertigo:0.04), (CALCITONIN:Vertigo:0.21), (CA LCITONIN SALMON:Vertigo:0.24), (CALCIUM ACETATE:Vertigo:0.7), (CALCIUM\MAGNESIUM\ZINC:Vertigo:0.01), (CANAGLIFLOZIN METFORMIN HYDROCHLORIDE:Vertigo:0.32), (CANAKINUMAB:Vertigo:0.21), (CANDESARTAN CILEXETILHYDROCHLOROTHIAZIDE:Vertigo:1.43), (CARVEDILOL PHOSPHATE:Vertigo:1.14), (CETUXIMAB:Vertigo:0.11), (CILASTATIN SODIUMIMIPENEM:Vertigo:0.04), (CRIZOTINIB:Vertigo:0.06), (DABIGATRAN:Vertigo:0.01), (DALTEPARIN:Vertigo:0.31), (DEFLAZACORT:Vertigo:0.69), (DIGITOXIN:Vertigo:0.05), (DIPYRIDAMOLE:Vertigo:0.09), (DIPYRONE:Vertigo:0.16), (DOCUSATE CALCIUM:Vertigo:0.82), (DONEPEZIL:Vertigo:0.26), (DOXYCYCLINE HYCLATE:Vertigo:0.09), (ECONAZOLE:Vertigo:0.15), (EMPAGLIFLOZINLINAGLIPTIN:V ertigo:0.66), (EPIRUBICIN:Vertigo:0.34), (EPLERENONE:Vertigo:0.53), (ESOMEPRAZOLE MAGNESIUMNAPROXEN:Vertigo:0.01), (ESZOPICLONE:Vertigo:1.4), (FERROUS GLUCONATE:Vertigo:0.04), (FLUTICASONE FUROATESALMETEROL XINAFOATE:Vertigo:0.72), (FORMOTEROL FUMARATE\MOMETASONE FUROATE:Vertigo:0.36), (FUSIDIC ACID:Vertigo:0.54), (HALOPERIDOL:Vertigo:0.38), (HERBALSTURMERIC:Vertigo:0.51), (HYDROXYCHLOROQUINE DIPHOSPHATE:Vertigo:0.22), (ILOPROST:Vertigo:0.23), (IMATINIB MESYLATE:Vertigo:1.2), (IMIPRAMINE HYDROCHLORIDE:Vertigo:0.29), (INDACATEROL:Vertigo:0.11), (INDAPAMIDEPERIND OPRIL:Vertigo:0.76), (INSULIN DEGLUDEC:Vertigo:0.11), (LERCANIDIPINE:Vertigo:1.36), (LERCANIDIPINE HYDROCHLORIDE:Vertigo:0.87), (LIDOCAINEPRILOCAINE:Vertigo:0.5), (LIOTHYRONI NE SODIUM:Vertigo:0.33), (LOMITAPIDE MESYLATE:Vertigo:0.25), (MEPOLIZUMAB:Vertigo:0.28), (MINOCYCLINEMINOCYCLIN E HYDROCHLORIDE:Vertigo:0.41), (MOXONIDINE:Vertigo:0.13), (MYCOPHENOLATE MOFETILMYCOPHENOLATE MOFETIL HYDROCHLORIDE:Vertigo:0.04), (NILOTINIB:Vertigo:1.55), (NORETHINDRONE:Vertigo:00.34), (OLODATEROL HYDROCHLORIDETIOTROPIUM BROMIDE MONOHYDRATE:Vertigo:0.55), (OSELTAMIVIR PHOSPHATE:Vertigo:0.13), (PATIROMER:Vertigo:0.18), (PAZOPANIB HYDROCHLORIDE:Vertigo:0.81), (PENTOSAN POLYSULFATE SODIUM:Vertigo:0.18), (PIOGLITAZONE:Vertigo:0.03), (PONATINIB:Vertigo:0.64), (PRAZO SIN:Vertigo:0.06), (PRAZOSIN HYDROCHLORIDE:Vertigo:0.19), (PSYLLIUM HUSK:Vertigo:0.37), (RALOXIFENE HYDROCHLORIDE:Vertigo:0.64), (CEREVISIAE:Vertigo:0.1), (SALMETEROL XINAFOATE:Vertigo:0.22), (SITAGLIPTIN:Vertigo:0.12), (TERAZOSIN HYDROCHLORIDE:Vertigo:0.19), (TERBUTALINE SULFATE:Vertigo:0.71), (TIOTROPIUM:Vertigo:0.53), (TROSPIUM CHLORIDE:Vertigo:0.18), (UMECLIDINIUM BROMIDE:Vertigo:0.22), (VANCOMYCIN HYDROCHLORIDE:Vertigo:0.06), (VILAZODONE HYDROCHLORIDE:Vertigo:0.6), (VITAMIN B:Vertigo:1.55), (ALFACALCIDOL:Viral infection:0.14), (AMIODARONE:Viral infection:0.77), (ANASTROZOLE:Viral infection:0.54), (ARMODAFINIL:Viral infection:0.37), (ATROPINE SULFATEDIPHENOXYLATE HYDROCHLORIDE:Viral infection:0.04), (BUDESONIDEFORMOTEROL FUMARATE:Viral infection:0.02), (BUPRENORPHINE HYDROCHLORIDE:Viral infection:0.26), (BUPRENORPHINE HYDROCHLORIDENALOXONE HYDROCHLORIDE:Viral infection:0.29), (BUSPIRONE HYDROCHLORIDE:Viral infection:0.43), (CALCIUM ACETATE:Viral infection:0.51), (CANDESARTAN CILEXETILHYDROCHLOROTHIAZIDE:Viral infection:0.39), (CLINDAMYCIN:Viral infection:0.16), (CLOZAPINE:Viral infection:0.39), (CORTISONEHYDROCORTISONE:Viral infection:0.49), (DACLIZUMAB:Viral infection:0.24), (DASATINIB:Viral infection:0.98), (DOCUSATE CALCIUM:Viral infection:0.01), (DOXAZOSINDOXAZOSIN MESYLATE:Viral infection:0.41), (DUTASTERIDE:Viral infection:0.13), (ECONAZOLE:Viral infection:0.05), (ELETRIPTAN HYDROBROMIDE:Viral infection:0.4), (ELOTUZUMAB:Viral infection:0.21), (ENALAPRIL MALEATE:Viral infection:0.38), (EPLERENONE:Viral infection:0.02), (ESZOPICLONE:Viral infection:0.85), (ETORICOXIB:Viral infection:0.08), (EVOLOCUMAB:Viral infection:0.61), (EZETIMIBESIMVASTATIN:Viral infection:0.06), (FERROUS SULFATEFERROUS SULFATE, DRIED:Viral infection:1.24), (FLUOXETINEFLUOXETINE HYDROCHLORIDE:Viral infection:0.25), (FLUTICASONE FUROATESALMETEROL XINAFOATE:Viral infection:0.35), (GLYCOPYRRONIUMINDACATEROL:Viral infection:0.18), (HYDRALAZINE HYDROCHLORIDE:Viral infection:0.39), (HYDROXYCHLOROQUINE DIPHOSPHATE:Viral infection:0.07), (IBRITUMOMAB TIUXETAN:Viral infection:0.05), (IMATINIB MESYLATE:Viral infection:0.64), (INDAPAMIDEPERINDOPRIL:Viral infection:0.34), (ISOSORBIDE:Viral infection:0.28), (ISOSORBIDE MONONITRATE:Viral infection:1.09), (IXAZOMIB:Viral infection:0.02), (LERCANIDIPINE HYDROCHLORIDE:Viral infection:0.27), (LISDEXAMFETAMINE DIMESYLATE:Viral infection:0.3), (LOVASTATIN:Viral infection:0.95), (METHADONE HYDROCHLORIDE:Viral infection:0.19), (METHENAMINE HIPPURATE:Viral infection:0.1), (NADOLOL:Viral infection:0.01), (NILOTINIB:Viral infection:1.27), (OXYGEN:Viral infection:0.49), (PANTOPRAZOLE MAGNESIUM:Viral infection:0.45), (PAROXETINE:Viral infection:0.26), (PAROXETINEPAROXETINE HYDROCHLORIDE:Viral infection:0.24), (PEGINTERFERON BETA-1A:Viral infection:0), (PHENOBARBITAL:Viral infection:0.3), (PONATINIB:Viral infection:0.62), (QUETIAPINE:Viral infection:0.19), (RABEPRAZOLE:Viral infection:0.06), (RANOLAZINE:Viral infection:0.42), (RISEDRONATE SODIUM:Viral infection:0.36), (ROFLUMILAST:Viral infection:0.17), (SACUBITRILVALSARTAN:Viral infection:0.65), (SECUKINUMAB:Viral infection:0.83), (SOTALOL:Viral infection:0.46), (SUMATRIPTAN:Viral infection:0.16), (TERIFLUNOMIDE:Viral infection:0.57), (TETRACYCLINE:Viral infection:0.18), (TIGECYCLINE:Viral infection:0.19), (TIOTROPIUM:Viral infection:0.13), (TIZANIDINE HYDROCHLORIDE:Viral infection:0.67), (TOFACITINIB CITRATE:Viral infection:0.22), (TOLTERODINE TARTRATE:Viral infection:0.06), (UMECLIDINIUM BROMIDEVILANTEROL TRIFENATATE:Viral infection:0.11), (VARENICLINE TARTRATE:Viral infection:0.54), (PYRAZINAMIDE:Viral test positive:0.52), (ACETAMINOPHENBUTALBITALCAFFEINE:Viral upper respiratory tract infection:0.02), (AMPHETAMINE ASPARTATEAMPHETAMINE SULFATEDEXTROAMPHETAMINE SACCHARATEDEXTROAMPHETAMINE SULFATE:Viral upper respiratory tract infection:0.44), (ARMODAFINIL:Viral upper respiratory tract infection:0.07), (BISACODYL OR DOCUSATE SODIUM:Viral upper respiratory tract infection:0.09), (BUDESONIDEFORMOTEROL FUMARATE:Viral upper respiratory tract infection:0.06), (CARFILZOMIB:Viral upper respiratory tract infection:0.47), (CEPHALEXIN:Viral upper respiratory tract infection:0.76), (CHOLESTYRAMINE:Viral upper respiratory tract infection:0.19), (CITALOPRAM HYDROBROMIDE:Viral upper respiratory tract infection:1.37), (CLINDAMYCINCLINDAMYCIN PHOSPHATE:Viral upper respiratory tract infection:0.99), (CLOTRIMAZOLE:Viral upper respiratory tract infection:0.13), (COLCHICINE:Viral upper respiratory tract infection:0.4), (CYCLOBENZAPRINE:Viral upper respiratory tract infection:0.27), (DASATINIB:Viral upper respiratory tract infection:0.53), (DIVALPROEX SODIUM:Viral upper respiratory tract infection:0.4), (DOXAZOSIN MESYLATE:Viral upper respiratory tract infection:0.17), (ESTROGENS, CONJUGATEDMEDROXYPROGESTERONE ACETATE:Viral upper respiratory tract infection:0.02), (ETHAMBUTOL HYDROCHLORIDE:Viral upper respiratory tract infection:0.93), (EVEROLIMUS:Viral upper respiratory tract infection:0.48), (EVOLOCUMAB:Viral upper respiratory tract infection:0.5), (FERROUS SULFATEFERROUS SULFATE, DRIED:Viral upper respiratory tract infection:0.86), (FLECAINIDE:Viral upper respiratory tract infection:0.21), (FLUTICASONE FUROATESALMETEROL XINAFOATE:Viral upper respiratory tract infection:0.16), (FORMOTEROL FUMARATE DIHYDRATE\MOMETASONE FUROATE:Viral upper respiratory tract infection:0.07), (GLYCOPYRRONIUMINDACATEROL:Viral upper respiratory tract infection:0.08), (HYDRALAZINE HYDROCHLORIDE:Viral upper respiratory tract infection:0.06), (HYDROCHLOROTHIAZIDEIRBESARTAN:Viral upper respiratory tract infection:0.13), (HYDROMORPHONE:Viral upper respiratory tract infection:0.15), (HYDROXYCHLOROQUINE DIPHOSPHATE:Viral upper respiratory tract infection:0.02), (HYDROXYZINE PAMOATE:Viral upper respiratory tract infection:0.08), (ILOPROST:Viral upper respiratory tract infection:0.03), (IMATINIB MESYLATE:Viral upper respiratory tract infection:0.21), (INDAPAMIDEPERINDOPRIL:Viral upper respiratory tract infection:0.04), (IPRATROPIUM:Viral upper respiratory tract infection:0.41), (LEDIPASVIRSOFOSBUVIR:Viral upper respiratory tract infection:0.38), (LERCANIDIPINE:Viral upper respiratory tract infection:0.09), (LISDEXAMFETAMINE DIMESYLATE:Viral upper respiratory tract infection:0.1), (LITHIUM CARBONATE:Viral upper respiratory tract infection:0.26), (MEPOLIZUMAB:Viral upper respiratory tract infection:0.22), (METAXALONE:Viral upper respiratory tract infection:0.03), (MORPHINE:Viral upper respiratory tract infection:0.43), (NEBIVOLOL:Viral upper respiratory tract infection:0.32), (NINTEDANIB:Viral upper respiratory tract infection:0.03), (NITROGLYCERIN:Viral upper respiratory tract infection:0.31), (OXCARBAZEPINE:Viral upper respiratory tract infection:0.19), (OXYGEN:Viral upper respiratory tract infection:0.93), (PAZOPANIB HYDROCHLORIDE:Viral upper respiratory tract infection:0.01), (PITAVASTATIN CALCIUM:Viral upper respiratory tract infection:0.14), (PRAMIPEXOLE DIHYDROCHLORIDE:Viral upper respiratory tract infection:0.19), (PREDNISOLONEPREDNISOLONE ACETATEPREDNISOLONE SODIUM SUCCINATE:Viral upper respiratory tract infection:0.83), (PROMETHAZINEPROMETHAZINE HYDROCHLORIDE:Viral upper respiratory tract infection:0.24), (RANOLAZINE:Viral upper respiratory tract infection:0.2), (SODIUM OXYBATE:Viral upper respiratory tract infection:0.44), (SOTALOL:Viral upper respiratory tract infection:0.11), (TERIFLUNOMIDE:Viral upper respiratory tract infection:0.64), (THALIDOMIDE:Viral upper respiratory tract infection:0.23), (TIZANIDINE HYDROCHLORIDE:Viral upper respiratory tract infection:0.26), (TOFACITINIB CITRATE:Viral upper respiratory tract infection:0.43), (TOLTERODINE TARTRATE:Viral upper respiratory tract infection:0.06), (AMIODARONE:Visual impairment:0.16), (BUDESONIDE:Visual impairment:0.13), (BUSPIRONE HYDROCHLORIDE:Visual impairment:0.21), (CALCIUM CARBONATECHOLECALCIFEROL:Visual impairment:0.36), (DILTIAZEM HYDROCHLORIDE:Visual impairment:0.73), (EVOLOCUMAB:Visual impairment:0.72), (IBRUTINIB:Visual impairment:0.67), (MORPHINE:Visual impairment:0.04), (OXYGEN:Visual impairment:0.13), (RABEPRAZOLE SODIUM:Visual impairment:0.12), (RALOXIFENE HYDROCHLORIDE:Visual impairment:0.38), (RANOLAZINE:Visual impairment:0.33), (SACUBITRILVALSARTAN:Visual impairment:0.61), (ALBUTEROL SULFATEIPRATROPIUM BROMIDE:Vitamin B complex deficiency:0.04), (AMIODARONE HYDROCHLORIDE:Vitamin B complex deficiency:0.29), (CANDESARTAN CILEXETIL:Vitamin B complex deficiency:0.23), (CEPHALEXIN:Vitamin B complex deficiency:0.33), (CHOLESTYRAMINE:Vitamin B complex deficiency:0.11), (CLONIDINE:Vitamin B complex deficiency:0.19), (CORTISONEHYDROCORTISONE:Vitamin B complex deficiency:0.25), (DASATINIB:Vitamin B complex deficiency:0.07), (DEXLANSOPRAZOLE:Vitamin B complex deficiency:0.92), (DILTIAZEM HYDROCHLORIDE:Vitamin B complex deficiency:0.55), (DIPHENHYDRAMINE:Vitamin B complex deficiency:0.54), (DUTASTERIDE:Vitamin B complex deficiency:0.07), (ENOXAPARIN:Vitamin B complex deficiency:0.48), (ESZOPICLONE:Vitamin B complex deficiency:0.17), (EVOLOCUMAB:Vitamin B complex deficiency:0.48), (EZETIMIBESIMVASTATIN:Vitamin B complex deficiency:0.02), (HYDROCHLOROTHIAZIDEVALSARTAN:Vitamin B complex deficiency:0.06), (INSULIN HUMAN:Vitamin B complex deficiency:0.14), (LATANOPROST:Vitamin B complex deficiency:0.08), (LEDIPASVIRSOFOSBUVIR:Vitamin B complex deficiency:0.38), (MELATONIN:Vitamin B complex deficiency:0.24), (METRONIDAZOLE:Vitamin B complex deficiency:0.84), (OLANZAPINE:Vitamin B complex deficiency:0.06), (OXYGEN:Vitamin B complex deficiency:0.29), (POLYETHYLENE GLYCOLS:Vitamin B complex deficiency:0.13), (PRAMIPEXOLE DIHYDROCHLORIDE:Vitamin B complex deficiency:0.38), (PRIMIDONE:Vitamin B complex deficiency:0.31), (PROMETHAZINEPROMETHAZINE HYDROCHLORIDE:Vitamin B complex deficiency:0.26), (RALOXIFENE HYDROCHLORIDE:Vitamin B complex deficiency:0.29), (RANOLAZINE:Vitamin B complex deficiency:0.68), (SACUBITRILVALSARTAN:Vitamin B complex deficiency:0.7), (SOTALOL:Vitamin B complex deficiency:0.27), (TAMSULOSIN HYDROCHLORIDE:Vitamin B complex deficiency:0.2), (TRIAMCINOLONE ACETONIDE:Vitamin B complex deficiency:0.08), (AMIODARONE:Vitamin B12 decreased:0.28), (ANASTROZOLE:Vitamin B12 decreased:0.35), (BUMETANIDE:Vitamin B12 decreased:0.07), (CANDESARTAN:Vitamin B12 decreased:0.1), (CEPHALEXIN:Vitamin B12 decreased:0.08), (CLINDAMYCINCLINDAMYCIN PHOSPHATE:Vitamin B12 decreased:0.28), (CLONIDINE:Vitamin B12 decreased:0.16), (CYCLOBENZAPRINE:Vitamin B12 decreased:0.25), (DICLOFENAC:Vitamin B12 decreased:0.04), (DIGOXIN:Vitamin B12 decreased:0.55), (DIVALPROEX SODIUM:Vitamin B12 decreased:0.32), (ENALAPRIL:Vitamin B12 decreased:1.15), (ENOXAPARIN:Vitamin B12 decreased:0.22), (EVOLOCUMAB:Vitamin B12 decreased:0.53), (LISDEXAMFETAMINE DIMESYLATE:Vitamin B12 decreased:0.12), (METAXALONE:Vitamin B12 decreased:0.38), (METRONIDAZOLE:Vitamin B12 decreased:1.12), (NAPROXEN SODIUM:Vitamin B12 decreased:0.1), (NYSTATIN:Vitamin B12 decreased:0.06), (PANTOPRAZOLE MAGNESIUM:Vitamin B12 decreased:0.02), (RABEPRAZOLE SODIUM:Vitamin B12 decreased:0.33), (RAMIPRIL:Vitamin B12 decreased:0.46), (RANOLAZINE:Vitamin B12 decreased:0.38), (SACUBITRILVALSARTAN:Vitamin B12 decreased:0.34), (SECUKINUMAB:Vitamin B12 decreased:0.05), (TOCILIZUMAB:Vitamin B12 decreased:0.09), (TRIAMCINOLONE ACETONIDE:Vitamin B12 decreased:0.13), (VARENICLINE TARTRATE:Vitamin B12 decreased:0.5), (VITAMIN B:Vitamin B12 decreased:0.29), (WARFARIN SODIUM:Vitamin B12 decreased:0.42), (ZOLPIDEMZOLPIDEM TARTRATE:Vitamin B12 decreased:0.37), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Vitamin B12 deficiency:0.09), (.ALPHA.1-PROTEINASE INHIBITOR HUMAN:Vitamin B12 deficiency:0.56), (ACETAMINOPHENOXYCODONEOXYCODONE HYDROCHLORIDE:Vitamin B12 deficiency:0.8), (ADO-TRASTUZUMAB EMTANSINE:Vitamin B12 deficiency:0.31), (AGALSIDASE BETA:Vitamin B12 deficiency:0.33), (ALBIGLUTIDE:Vitamin B12 deficiency:0.09), (ALCOHOL:Vitamin B12 deficiency:0.64), (ALISKIREN HEMIFUMARATE:Vitamin B12 deficiency:0.2), (ALUMINUM HYDROXIDEDIMETHICONEMAGNESIUM HYDROXIDE:Vitamin B12 deficiency:0.51), (AMILORIDE HYDROCHLORIDEHYDROCHLOROTHIAZIDE:Vitamin B12 deficiency:0.2), (AMINO ACIDSDEXTROSEELECTROLYTES NOS:Vitamin B12 deficiency:0.62), (AMISULPRIDE:Vitamin B12 deficiency:0.6), (AMLODIPINE BESYLATEHYDROCHLOROTHIAZIDEOLMESARTAN MEDOXOMIL:Vitamin B12 deficiency:0.34), (AMLODIPINEVALSARTAN:Vitamin B12 deficiency:0.29), (ANTIHISTAMINES NOS:Vitamin B12 deficiency:0.15), (ARIPIPRAZOLE LAUROXIL:Vitamin B12 deficiency:0.1), (AZILSARTAN KAMEDOXOMIL:Vitamin B12 deficiency:0.04), (BENAZEPRILBENAZEPRIL HYDROCHLORIDE:Vitamin B12 deficiency:0.59), (BENSERAZIDELEVODOPA:Vitamin B12 deficiency:0.17), (BICALUTAMIDE:Vitamin B12 deficiency:0.77), (BRIMONIDINE TARTRATETIMOLOL MALEATE:Vitamin B12 deficiency:0.01), (BROMAZEPAM:Vitamin B12 deficiency:0.74), (BUTYLSCOPOLAMINE BROMIDE:Vitamin B12 deficiency:0.36), (CALCIPOTRIENE:Vitamin B12 deficiency:0.01), (CALCIUM ACETATE:Vitamin B12 deficiency:0.93), (CALCIUM CARBONATEVITAMIN D:Vitamin B12 deficiency:0.46), (CALCIUM POLYCARBOPHIL:Vitamin B12 deficiency:0.06), (CALCIUM\MAGNESIUM:Vitamin B12 deficiency:0.43), (CALCIUM\MAGNESIUM\ZINC:Vitamin B12 deficiency:0.33), (CANAGLIFLOZINMETFORMIN HYDROCHLORIDE:Vitamin B12 deficiency:0.73), (CAPTOPRIL:Vitamin B12 deficiency:0.71), (CEFUROXIME AXETIL:Vitamin B12 deficiency:0.03), (CETIRIZINE HYDROCHLORIDEPSEUDOEPHEDRINE HYDROCHLORIDE:Vitamin B12 deficiency:0.29), (CHONDROITIN SULFATE (BOVINE)GLUCOSAMINE HYDROCHLORIDE:Vitamin B12 deficiency:0.55), (CLINDAMYCIN:Vitamin B12 deficiency:0.86), (CLOMIPRAMINE HYDROCHLORIDE:Vitamin B12 deficiency:0.04), (COD LIVER OIL:Vitamin B12 deficiency:0.37), (DACLIZUMAB:Vitamin B12 deficiency:0.83), (DANAZOL:Vitamin B12 deficiency:0.64), (DASATINIB:Vitamin B12 deficiency:1.65), (DIGITOXIN:Vitamin B12 deficiency:0.08), (DIPYRONE:Vitamin B12 deficiency:0.33), (DOXEPIN:Vitamin B12 deficiency:0.7), (DOXEPIN HYDROCHLORIDE:Vitamin B12 deficiency:0.61), (DOXYLAMINE SUCCINATE:Vitamin B12 deficiency:0.05), (ECONAZOLE:Vitamin B12 deficiency:0.24), (EDOXABAN TOSYLATE:Vitamin B12 deficiency:0.64), (ESOMEPRAZOLE MAGNESIUMNAPROXEN:Vitamin B12 deficiency:0.37), (ETORICOXIB:Vitamin B12 deficiency:0.49), (EVOLOCUMAB:Vitamin B12 deficiency:1.47), (FLURAZEPAMFLURAZEPAM HYDROCHLORIDE:Vitamin B12 deficiency:0.06), (FORMOTEROL:Vitamin B12 deficiency:0.2), (FORMOTEROL FUMARATE:Vitamin B12 deficiency:0.77), (FORMOTEROL FUMARATE\MOMETASONE FUROATE:Vitamin B12 deficiency:0.37), (FULVESTRANT:Vitamin B12 deficiency:0.46), (FUSIDIC ACID:Vitamin B12 deficiency:0.5), (GLYCOPYRROLATE:Vitamin B12 deficiency:0.11), (GUANFACINE:Vitamin B12 deficiency:0.3), (HERBALS:Vitamin B12 deficiency:0.14), (HYDROCHLOROTHIAZIDETELMISARTAN:Vitamin B12 deficiency:0.65), (HYDROCODONE BITARTRATEIBUPROFEN:Vitamin B12 deficiency:0.17), (HYDROCORTISONE ACETATE:Vitamin B12 deficiency:0.19), (ILOPROST:Vitamin B12 deficiency:0.47), (INDACATEROL:Vitamin B12 deficiency:0.04), (INDAPAMIDEPERINDOPRIL:Vitamin B12 deficiency:0.96), (INSULIN DEGLUDEC:Vitamin B12 deficiency:0.42), (INTERFERON ALFA-2A:Vitamin B12 deficiency:0.07), (IODINE:Vitamin B12 deficiency:0.11), (LABETALOL HYDROCHLORIDE:Vitamin B12 deficiency:0.08), (LEDIPASVIRSOFOSBUVIR:Vitamin B12 deficiency:0.69), (LOMITAPIDE MESYLATE:Vitamin B12 deficiency:0.48), (MEFENAMIC ACID:Vitamin B12 deficiency:0.06), (MEPOLIZUMAB:Vitamin B12 deficiency:0.22), (MEROPENEM:Vitamin B12 deficiency:0.07), (METFORMIN HYDROCHLORIDEVILDAGLIPTIN:Vitamin B12 deficiency:0.1), (METHENAMINE HIPPURATE:Vitamin B12 deficiency:1.22), (MUPIROCIN:Vitamin B12 deficiency:0.37), (NADROPARIN CALCIUM:Vitamin B12 deficiency:0.19), (NATEGLINIDE:Vitamin B12 deficiency:0.3), (NIRAPARIB:Vitamin B12 deficiency:0.37), (ORPHENADRINE:Vitamin B12 deficiency:0.02), (OSELTAMIVIR PHOSPHATE:Vitamin B12 deficiency:0.07), (PALIPERIDONE PALMITATE:Vitamin B12 deficiency:0.27), (PATIROMER:Vitamin B12 deficiency:0.32), (PAZOPANIB HYDROCHLORIDE:Vitamin B12 deficiency:0.63), (PEGFILGRASTIM:Vitamin B12 deficiency:0.78), (PEGINTERFERON BETA-1A:Vitamin B12 deficiency:0.91), (PEMETREXED DISODIUM:Vitamin B12 deficiency:0.81), (PENICILLIN:Vitamin B12 deficiency:1.02), (PENTOXIFYLLINE:Vitamin B12 deficiency:0.02), (PHENYTOIN SODIUM:Vitamin B12 deficiency:0.52), (PILOCARPINE:Vitamin B12 deficiency:0.06), (PONATINIB:Vitamin B12 deficiency:1.3), (PRAZOSIN HYDROCHLORIDE:Vitamin B12 deficiency:0.34), (PREDNISOLONEPREDNISOLONE ACETATEPREDNISOLONE SODIUM SUCCINATE:Vitamin B12 deficiency:0.3), (PROPAFENONE HYDROCHLORIDE:Vitamin B12 deficiency:0.22), (PYRIDOSTIGMINE BROMIDE:Vitamin B12 deficiency:0.02), (QUINAPRIL:Vitamin B12 deficiency:0.14), (RALOXIFENE HYDROCHLORIDE:Vitamin B12 deficiency:0.72), (REGORAFENIB:Vitamin B12 deficiency:0.23), (RIZATRIPTAN:Vitamin B12 deficiency:0.17), (SCOPOLAMINE:Vitamin B12 deficiency:1.14), (SECUKINUMAB:Vitamin B12 deficiency:0.99), (SELEGILINE:Vitamin B12 deficiency:0.07), (TERBINAFINE HYDROCHLORIDE:Vitamin B12 deficiency:0.56), (TERBUTALINE SULFATE:Vitamin B12 deficiency:1.31), (TINZAPARIN SODIUM:Vitamin B12 deficiency:0.32), (UMECLIDINIUM BROMIDEVILANTEROL TRIFENATATE:Vitamin B12 deficiency:0.94), (URSODIOL:Vitamin B12 deficiency:0.47), (PYRAZINAMIDE:Vitamin B6 deficiency:0.57), (AMIODARONE HYDROCHLORIDE:Vitamin D:0.07), (BISOPROLOL FUMARATE:Vitamin D:0.52), (CANDESARTAN CILEXETIL:Vitamin D:0.12), (COLCHICINE:Vitamin D:0.04), (DALFAMPRIDINE:Vitamin D:0.02), (DEVICE:Vitamin D:0.53), (DEXLANSOPRAZOLE:Vitamin D:0.63), (ENALAPRIL MALEATE:Vitamin D:0.05), (EVOLOCUMAB:Vitamin D:0.67), (IBRUTINIB:Vitamin D:0.41), (IRBESARTAN:Vitamin D:0.16), (ISOSORBIDE MONONITRATE:Vitamin D:0.03), (LENALIDOMIDE:Vitamin D:0.11), (LERCANIDIPINE:Vitamin D:0.22), (MELATONIN:Vitamin D:0), (METHYLPREDNISOLONE ACETATE:Vitamin D:0.21), (METRONIDAZOLE:Vitamin D:0.33), (OMEPRAZOLE MAGNESIUM:Vitamin D:0.46), (PANTOPRAZOLE MAGNESIUM:Vitamin D:0.04), (PHENOBARBITAL:Vitamin D:0.21), (PRAVASTATIN SODIUM:Vitamin D:0.48), (RANOLAZINE:Vitamin D:0.41), (SACUBITRILVALSARTAN:Vitamin D:0.23), (SECUKINUMAB:Vitamin D:0.26), (TOFACITINIB CITRATE:Vitamin D:0.21), (TRAMADOL HYDROCHLORIDE:Vitamin D:0.07), (ZOLPIDEM TARTRATE:Vitamin D:0.08), (.ALPHA.1-PROTEINASE INHIBITOR HUMAN:Vitamin D decreased:0.81), (ABIRATERONE ACETATE:Vitamin D decreased:0.16), (ACARBOSE:Vitamin D decreased:0.31), (ACETAMINOPHENOXYCODONE HYDROCHLORIDEOXYCODONE TEREPHTHALATE:Vitamin D decreased:0.54), (ACETAMINOPHENOXYCODONEOXYCODONE HYDROCHLORIDE:Vitamin D decreased:0.65), (ACETAZOLAMIDE:Vitamin D decreased:0.48), (ALBIGLUTIDE:Vitamin D decreased:0.31), (ALCOHOL:Vitamin D decreased:0.46), (AMLODIPINE BESYLATEHYDROCHLOROTHIAZIDEOLMESARTAN MEDOXOMIL:Vitamin D decreased:0.46), (AZELASTINE:Vitamin D decreased:0.14), (BETAMETHASONE DIPROPIONATECALCIPOTRIENECALCIPOTRIENE HYDRATE:Vitamin D decreased:0.07), (BISOPROLOL:Vitamin D decreased:0.73), (BRINZOLAMIDE:Vitamin D decreased:0.14), (BUPRENORPHINE HYDROCHLORIDE:Vitamin D decreased:0.53), (CANAGLIFLOZINMETFORMIN HYDROCHLORIDE:Vitamin D decreased:0.54), (CANAKINUMAB:Vitamin D decreased:0.24), (CAPTOPRIL:Vitamin D decreased:0.45), (CARFILZOMIB:Vitamin D decreased:0.56), (CHOLESTYRAMINE:Vitamin D decreased:1.4), (CILOSTAZOL:Vitamin D decreased:0.15), (CLINDAMYCINCLINDAMYCIN PHOSPHATE:Vitamin D decreased:1.07), (COD LIVER OIL:Vitamin D decreased:0.34), (COLCHICINE:Vitamin D decreased:0.88), (CORTICOTROPIN:Vitamin D decreased:0.49), (DABIGATRAN:Vitamin D decreased:0.05), (DACLIZUMAB:Vitamin D decreased:0.74), (DANAZOL:Vitamin D decreased:0.42), (DASABUVIROMBITASVIRPARITAPREVIRRITONAVIR:Vitamin D decreased:0.04), (DASATINIB:Vitamin D decreased:1.36), (DEFLAZACORT:Vitamin D decreased:0.1), (DESONIDE:Vitamin D decreased:0.15), (DIMENHYDRINATE:Vitamin D decreased:0.7), (DIPYRONE:Vitamin D decreased:0.01), (EMPAGLIFLOZINLINAGLIPTIN:Vitamin D decreased:0.94), (EPLERENONE:Vitamin D decreased:0.2), (ESOMEPRAZOLE MAGNESIUMNAPROXEN:Vitamin D decreased:0.16), (ESTROGENS:Vitamin D decreased:0.06), (ESZOPICLONE:Vitamin D decreased:1.5), (ETORICOXIB:Vitamin D decreased:0.28), (EVOLOCUMAB:Vitamin D decreased:1.91), (FENOFIBRIC ACID:Vitamin D decreased:0.24), (FLUDROCORTISONE ACETATE:Vitamin D decreased:0.13), (FORMOTEROL FUMARATE\MOMETASONE FUROATE:Vitamin D decreased:0.34), (FUSIDIC ACID:Vitamin D decreased:0.07), (GLYCOPYRRONIUMINDACATEROL:Vitamin D decreased:0.41), (GUANFACINE:Vitamin D decreased:0.21), (HYDROCHLOROTHIAZIDEIRBESARTAN:Vitamin D decreased:0.59), (HYDROCODONE BITARTRATEIBUPROFEN:Vitamin D decreased:0.08), (HYDROMORPHONE:Vitamin D decreased:0.67), (ILOPROST:Vitamin D decreased:0.65), (IMATINIB MESYLATE:Vitamin D decreased:1.23), (IMIPRAMINE HYDROCHLORIDE:Vitamin D decreased:0.58), (INDAPAMIDEPERINDOPRIL:Vitamin D decreased:0.35), (LACOSAMIDE:Vitamin D decreased:0.01), (LACTASE:Vitamin D decreased:0.13), (LEVALBUTEROL HYDROCHLORIDE:Vitamin D decreased:1.35), (LITHIUM CARBONATE:Vitamin D decreased:1.27), (LOMITAPIDE MESYLATE:Vitamin D decreased:0.63), (MEGESTROL ACETATE:Vitamin D decreased:0.34), (MEPOLIZUMAB:Vitamin D decreased:0.12), (NEBIVOLOL:Vitamin D decreased:0.13), (NICOTINE:Vitamin D decreased:0.18), (NILOTINIB:Vitamin D decreased:1.1), (NITROFURANTOINNITROFURANTOIN MONOHYDRATE:Vitamin D decreased:0.3), (NORETHINDRONE:Vitamin D decreased:0.86), (OSPEMIFENE:Vitamin D decreased:0.25), (PARATHYROID HORMONE:Vitamin D decreased:0.01), (PATIROMER:Vitamin D decreased:0.27), (PAZOPANIB HYDROCHLORIDE:Vitamin D decreased:0.79), (PEGINTERFERON BETA-1A:Vitamin D decreased:0.84), (PENICILLIN:Vitamin D decreased:0.73), (PIMECROLIMUS:Vitamin D decreased:0.01), (PIOGLITAZONE:Vitamin D decreased:0.02), (PIROXICAM:Vitamin D decreased:0.31), (PRAZOSIN HYDROCHLORIDE:Vitamin D decreased:0.28), (PRIMIDONE:Vitamin D decreased:1.43), (QUINAPRIL:Vitamin D decreased:0.06), (RIOCIGUAT:Vitamin D decreased:0.47), (RIVASTIGMINE TARTRATE:Vitamin D decreased:0.01), (RIZATRIPTAN:Vitamin D decreased:0.07), (SALMETEROL XINAFOATE:Vitamin D decreased:0.17), (SULFAMETHOXAZOLE:Vitamin D decreased:0.62), (TETRACYCLINE:Vitamin D decreased:1.22), (TIMOLOL:Vitamin D decreased:0.64), (TIOTROPIUM:Vitamin D decreased:0.25), (UMECLIDINIUM BROMIDEVILANTEROL TRIFENATATE:Vitamin D decreased:1.3), (VALGANCICLOVIR HYDROCHLORIDE:Vitamin D decreased:0.27), (ZAFIRLUKAST:Vitamin D decreased:0.16), (ZIPRASIDONE HYDROCHLORIDEZIPRASIDONE MESYLATE:Vitamin D decreased:0.1), (ZOPICLONE:Vitamin D decreased:0.73), (.ALPHA.1-PROTEINASE INHIBITOR HUMAN:Vitamin D deficiency:1.38), (ABOBOTULINUMTOXINA:Vitamin D deficiency:0.24), (ACARBOSE:Vitamin D deficiency:1.5), (ACEBUTOLOL HYDROCHLORIDE:Vitamin D deficiency:0.79), (ACETAMINOPHEN\CAFFEINE:Vitamin D deficiency:0.49), (ACETAMINOPHEN\CAFFEINEOPIUM:Vitamin D deficiency:1.03), (ACETAMINOPHENMETHOCARBAMOL:Vitamin D deficiency:0.1), (ACETAMINOPHENPROPOXYPHENE NAPSYLATE:Vitamin D deficiency:0.63), (ACITRETIN:Vitamin D deficiency:1.04), (ACTIVATED CHARCOAL:Vitamin D deficiency:0.61), (ALCOHOL:Vitamin D deficiency:0.7), (AMIKACIN SULFATE:Vitamin D deficiency:0.25), (AMINO ACIDSDEXTROSEELECTROLYTES NOS:Vitamin D deficiency:1.69), (AMMONIUM CHLORIDEDEXTROMETHORPHAN\SODIUM CITRATE:Vitamin D deficiency:0.08), (AMOXICILLINCLARITHROMYCINESOMEPRAZOLE:Vitamin D deficiency:0.5), (ANHYDROUS CITRIC ACIDASPIRIN\SODIUM BICARBONATE:Vitamin D deficiency:0.17), (ANTIHISTAMINES NOS:Vitamin D deficiency:0.65), (ARIPIPRAZOLE LAUROXIL:Vitamin D deficiency:0.52), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:Vitamin D deficiency:0.34), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE MONONITRATE:Vitamin D deficiency:0.13), (ASPIRINCLOPIDOGREL:Vitamin D deficiency:0.29), (ASUNAPREVIR:Vitamin D deficiency:0.36), (ATOVAQUONE:Vitamin D deficiency:0.96), (ATROPINE:Vitamin D deficiency:0.33), (ATROPINE SULFATE:Vitamin D deficiency:0.15), (AZILSARTAN KAMEDOXOMIL:Vitamin D deficiency:0.78), (BACITRACIN:Vitamin D deficiency:0.58), (BECLOMETHASONEFORMOTEROL:Vitamin D deficiency:0.3), (BETAMETHASONE DIPROPIONATECALCIPOTRIENECALCIPOTRIENE HYDRATE:Vitamin D deficiency:1.21), (BETAMETHASONE SODIUM PHOSPHATE:Vitamin D deficiency:0.33), (BETAXOLOL HYDROCHLORIDE:Vitamin D deficiency:0.19), (BETHANECHOL CHLORIDE:Vitamin D deficiency:0.65), (BICALUTAMIDE:Vitamin D deficiency:1.34), (BIPERIDEN HYDROCHLORIDE:Vitamin D deficiency:0.01), (BLINATUMOMAB:Vitamin D deficiency:0.5), (BOCEPREVIR:Vitamin D deficiency:0.55), (BOTULINUM TOXIN TYPE A:Vitamin D deficiency:0.21), (BUTALBITAL:Vitamin D deficiency:0.12), (CABOZANTINIB S-MALATE:Vitamin D deficiency:0.1), (CALCIUM CARBONATEERGOCALCIFEROL:Vitamin D deficiency:0.07), (CALCIUM CHLORIDEDEXTROSE MONOHYDRATEMAGNESIUM CHLORIDE\SODIUM CHLORIDE\SODIUM LACTATE:Vitamin D deficiency:0.03), (CALCIUM\MAGNESIUM:Vitamin D deficiency:1.18), (CALCIUM\MAGNESIUM\ZINC:Vitamin D deficiency:1.36), (CARBIDOPAENTACAPONELEVODOPA:Vitamin D deficiency:0.22), (CARBOHYDRATESFATTY ACIDSMINERALSPROTEINVITAMINS:Vitamin D deficiency:0.25), (CASPOFUNGIN:Vitamin D deficiency:0.72), (CEFDINIR:Vitamin D deficiency:0.53), (CEFEPIMECEFEPIME HYDROCHLORIDE:Vitamin D deficiency:0.32), (CEFOTAXIMECEFOTAXIME SODIUM:Vitamin D deficiency:0.05), (CEFPODOXIME PROXETIL:Vitamin D deficiency:0.23), (CEFPROZIL:Vitamin D deficiency:0.11), (CERTOPARIN SODIUM:Vitamin D deficiency:0.2), (CETUXIMAB:Vitamin D deficiency:0.54), (CHLORAMBUCIL:Vitamin D deficiency:0.49), (CHLORHEXIDINE:Vitamin D deficiency:0.96), (CHLOROTHIAZIDE:Vitamin D deficiency:1.12), (CHOLESTEROL:Vitamin D deficiency:0.08), (CHONDROITIN SULFATE (CHICKEN):Vitamin D deficiency:0.04), (CIPROFIBRATE:Vitamin D deficiency:0.91), (CLIKSTAR:Vitamin D deficiency:0.42), (CLOSTRIDIUM BUTYRICUM SPORES STRAIN M-55:Vitamin D deficiency:0.17), (CLOXAZOLAM:Vitamin D deficiency:0.18), (CODEINE PHOSPHATE:Vitamin D deficiency:0.38), (COLISTIMETHATE SODIUM:Vitamin D deficiency:0.48), (COLLAGEN:Vitamin D deficiency:0.11), (CYPROTERONE ACETATEETHINYL ESTRADIOL:Vitamin D deficiency:0.5), (DANAZOL:Vitamin D deficiency:1.87), (DANTROLENE SODIUM:Vitamin D deficiency:0.53), (DEGARELIX:Vitamin D deficiency:0.07), (DEGARELIX ACETATE:Vitamin D deficiency:0.09), (DEXAMETHASONE ACETATE:Vitamin D deficiency:0.38), (DIPYRIDAMOLE:Vitamin D deficiency:1.18), (DIPYRONEPROMETHAZINE HYDROCHLORIDE:Vitamin D deficiency:0.62), (DOBUTAMINEDOBUTAMINE HYDROCHLORIDE:Vitamin D deficiency:0.24), (DOLASETRON MESYLATE:Vitamin D deficiency:0.56), (DOPAMINEDOPAMINE HYDROCHLORIDE:Vitamin D deficiency:0.13), (ELBASVIRGRAZOPREVIR:Vitamin D deficiency:0.32), (ELOTUZUMAB:Vitamin D deficiency:0.72), (ELTROMBOPAG OLAMINE:Vitamin D deficiency:0.84), (EMPAGLIFLOZINMETFORMIN HYDROCHLORIDE:Vitamin D deficiency:0.08), (EMTRICITABINETENOFOVIR:Vitamin D deficiency:0.27), (EPIRUBICIN:Vitamin D deficiency:0.9), (ERIBULIN:Vitamin D deficiency:0.05), (ERTAPENEM SODIUM:Vitamin D deficiency:0.41), (ESTRADIOLNORETHINDRONE ACETATE:Vitamin D deficiency:0.71), (ESTRIOL:Vitamin D deficiency:0.43), (ESTROGENS:Vitamin D deficiency:0.81), (ETHACRYNIC ACID:Vitamin D deficiency:0.14), (FELBAMATE:Vitamin D deficiency:0.08), (FENOTEROL HYDROBROMIDEIPRATROPIUM BROMIDE:Vitamin D deficiency:0.82), (FLUNARIZINE HYDROCHLORIDE:Vitamin D deficiency:0.17), (FLUOROMETHOLONE:Vitamin D deficiency:0.49), (FLURAZEPAMFLURAZEPAM HYDROCHLORIDE:Vitamin D deficiency:0.46), (FORMOTEROL:Vitamin D deficiency:1.96), (FORMOTEROL FUMARATE\MOMETASONE FUROATE:Vitamin D deficiency:1.11), (FROVATRIPTAN SUCCINATE:Vitamin D deficiency:0.32), (FUSIDIC ACID:Vitamin D deficiency:1.95), (GIMERACILOTERACILTEGAFUR:Vitamin D deficiency:0.07), (GLIPIZIDEMETFORMIN HYDROCHLORIDE:Vitamin D deficiency:0.34), (GLYBURIDEMETFORMIN HYDROCHLORIDE:Vitamin D deficiency:0.06), (GLYCOPYRROLATE:Vitamin D deficiency:1.32), (GLYCOPYRROLATEINDACATEROL MALEATE:Vitamin D deficiency:0.65), (GLYCOPYRRONIUM:Vitamin D deficiency:0.82), (HORSE CHESTNUT:Vitamin D deficiency:0.01), (HYALURONATE SODIUM:Vitamin D deficiency:0.47), (HYDROCHLOROTHIAZIDEMETOPROLOL SUCCINATE:Vitamin D deficiency:0.09), (HYDROCHLOROTHIAZIDERAMIPRIL:Vitamin D deficiency:0.26), (HYOSCYAMINE SULFATEMETHENAMINEMETHYLENE BLUEPHENYL SALICYLATE\SODIUM PHOSPHATE, MONOBASIC, MONOHYDRATE:Vitamin D deficiency:0.06), (IDELALISIB:Vitamin D deficiency:0.69), (ILOPROST:Vitamin D deficiency:1.01), (IMIQUIMOD:Vitamin D deficiency:0.42), (INTERFERON:Vitamin D deficiency:1.36), (INTERFERON ALFA-2A:Vitamin D deficiency:1.29), (INTERFERON ALFA-2B:Vitamin D deficiency:0.73), (INTERFERON GAMMA-1B:Vitamin D deficiency:0.67), (IRINOTECAN HYDROCHLORIDE:Vitamin D deficiency:0.51), (IRON POLYMALTOSE:Vitamin D deficiency:0.03), (ISOPROPYL ALCOHOLLEUPROLIDE ACETATENORETHINDRONE ACETATE:Vitamin D deficiency:0.36), (IVABRADINE HYDROCHLORIDE:Vitamin D deficiency:0.34), (IXAZOMIB:Vitamin D deficiency:0.85), (ACIDOPHILUSLACTOBACILLUS DELBRUECKII SUBSP. BULGARICUS:Vitamin D deficiency:0.21), (LINSIDOMINE:Vitamin D deficiency:0.04), (LUTETIUM LU-177:Vitamin D deficiency:0.37), (MARAVIROC:Vitamin D deficiency:0.75), (MELPERONE:Vitamin D deficiency:0.09), (MEPOLIZUMAB:Vitamin D deficiency:0.97), (MESNA:Vitamin D deficiency:0.54), (METFORMIN HYDROCHLORIDESAXAGLIPTIN:Vitamin D deficiency:0.12), (METFORMIN PAMOATE:Vitamin D deficiency:0.29), (METFORMINSITAGLIPTIN:Vitamin D deficiency:0.03), (METHENAMINE MANDELATE:Vitamin D deficiency:0.17), (MIANSERIN:Vitamin D deficiency:0.03), (MIDODRINE HYDROCHLORIDE:Vitamin D deficiency:1.08), (MOEXIPRIL HYDROCHLORIDE:Vitamin D deficiency:0.34), (NALOXONE:Vitamin D deficiency:0.69), (NARATRIPTAN HYDROCHLORIDE:Vitamin D deficiency:0.51), (NEOMYCIN:Vitamin D deficiency:0.45), (NICARDIPINE:Vitamin D deficiency:1.22), (NIRAPARIB:Vitamin D deficiency:0.91), (OLOPATADINE:Vitamin D deficiency:0.61), (ORPHENADRINE:Vitamin D deficiency:0.75), (OSELTAMIVIR:Vitamin D deficiency:0.82), (OXALIPLATIN:Vitamin D deficiency:0.02), (OXYBUTYNINOXYBUTYNIN CHLORIDE:Vitamin D deficiency:0.58), (PANITUMUMAB:Vitamin D deficiency:0.1), (PATIROMER:Vitamin D deficiency:1.13), (PEGVISOMANT:Vitamin D deficiency:0.65), (PEMETREXED:Vitamin D deficiency:0.56), (PEMETREXED DISODIUM:Vitamin D deficiency:1), (PENTOSTATIN:Vitamin D deficiency:0.23), (PHENAZOPYRIDINE HYDROCHLORIDE:Vitamin D deficiency:0.37), (PIRFENIDONE:Vitamin D deficiency:0.26), (POLYETHYLENE GLYCOL 3350POTASSIUM CHLORIDE\SODIUM BICARBONATE\SODIUM CHLORIDE\SODIUM SULFATE ANHYDROUS:Vitamin D deficiency:0.53), (PRAZEPAM:Vitamin D deficiency:0.08), (PREDNISOLONEPREDNISOLONE ACETATEPREDNISOLONE SODIUM SUCCINATE:Vitamin D deficiency:1.09), (PRISTINAMYCIN:Vitamin D deficiency:0.5), (QUINACRINE:Vitamin D deficiency:0.27), (RADIATION THERAPY:Vitamin D deficiency:1.03), (RAMUCIRUMAB:Vitamin D deficiency:0.06), (SAPROPTERIN DIHYDROCHLORIDE:Vitamin D deficiency:0.14), (SELEGILINE HYDROCHLORIDE:Vitamin D deficiency:0.3), (SIBUTRAMINE HYDROCHLORIDE:Vitamin D deficiency:0.29), (SOLIFENACIN:Vitamin D deficiency:0.02), (SORBITOL:Vitamin D deficiency:0.1), (SOTALOL HYDROCHLORIDE:Vitamin D deficiency:0.69), (STREPTOMYCIN\STREPTOMYCIN SULFATE:Vitamin D deficiency:0.01), (SUVOREXANT:Vitamin D deficiency:0.09), (TEMSIROLIMUS:Vitamin D deficiency:0.03), (TERBUTALINE:Vitamin D deficiency:0.74), (TERBUTALINE SULFATE:Vitamin D deficiency:2.31), (TESAMORELIN:Vitamin D deficiency:0.14), (TETRABENAZINE:Vitamin D deficiency:0.04), (TETRACYCLINE:Vitamin D deficiency:2.04), (THEOPHYLLINE:Vitamin D deficiency:0.39), (THIOCTIC ACID:Vitamin D deficiency:0.67), (TIBOLONE:Vitamin D deficiency:0.41), (TICLOPIDINE:Vitamin D deficiency:0.21), (TIMOLOL MALEATE:Vitamin D deficiency:1.08), (TIMOLOLTRAVOPROST:Vitamin D deficiency:0.04), (TIPIRACIL HYDROCHLORIDETRIFLURIDINE:Vitamin D deficiency:0.06), (TOLTERODINE:Vitamin D deficiency:0.75), (TRAMETINIB DIMETHYL SULFOXIDE:Vitamin D deficiency:0.15), (TRIHEXYPHENIDYL:Vitamin D deficiency:0.28), (TRIPTORELIN PAMOATE:Vitamin D deficiency:0.49), (TROSPIUM:Vitamin D deficiency:0.26), (YEAST:Vitamin D deficiency:0.53), (ZILEUTON:Vitamin D deficiency:0.48), (.BETA.-HYDROXYISOVALERIC ACIDARGININEGLUTAMINE:Vitamin supplementation:0.08), (ACECLOFENAC:Vitamin supplementation:0.51), (ACETAMINOPHENBUTALBITALCAFFEINECODEINE PHOSPHATE:Vitamin supplementation:0.38), (ACETAMINOPHEN\CAFFEINECARISOPRODOLDICLOFENAC SODIUM:Vitamin supplementation:0.38), (ACETAMINOPHENCHLORPHENIRAMINE MALEATEPSEUDOEPHEDRINE HYDROCHLORIDE:Vitamin supplementation:0.48), (ACETAMINOPHENMETHOCARBAMOL:Vitamin supplementation:0.85), (ADO-TRASTUZUMAB EMTANSINE:Vitamin supplementation:1.82), (ALBENDAZOLE:Vitamin supplementation:0.19), (ALBUTEROLGUAIFENESIN:Vitamin supplementation:0.12), (ALBUTEROLIPRATROPIUM BROMIDE:Vitamin supplementation:0.26), (ALDESLEUKIN:Vitamin supplementation:0.44), (ALGLUCOSIDASE ALFA:Vitamin supplementation:1.54), (ALISKIREN HEMIFUMARATEHYDROCHLOROTHIAZIDE:Vitamin supplementation:0.55), (ALMOTRIPTAN MALATE:Vitamin supplementation:0.48), (ALUMINUM HYDROXIDEMAGNESIUM OXIDEOXETHAZAINE:Vitamin supplementation:0.11), (AMOXICILLINCLARITHROMYCINESOMEPRAZOLE:Vitamin supplementation:1.17), (ANAGRELIDE:Vitamin supplementation:0.31), (ANAKINRA:Vitamin supplementation:0.1), (APPLE CIDER VINEGAR:Vitamin supplementation:0.18), (ARIPIPRAZOLE LAUROXIL:Vitamin supplementation:0.51), (ASCORBIC ACID/CHROMIUM/ SELENIUM/VANADYL SULFATE:Vitamin supplementation:0.46), (ASPIRIN\CAFFEINESALICYLAMIDE:Vitamin supplementation:0.34), (ASPIRINCLOPIDOGREL:Vitamin supplementation:0.23), (ATAZANAVIRRITONAVIR:Vitamin supplementation:0.18), (AZITHROMYCIN MONOHYDRATE:Vitamin supplementation:1.03), (BACITRACIN:Vitamin supplementation:1.95), (BACITRACINNEOMYCINPOLYMYXIN B:Vitamin supplementation:0.06), (BAMIFYLLINE HYDROCHLORIDE:Vitamin supplementation:0.81), (BECLOMETHASONE:Vitamin supplementation:1.85), (BECLOMETHASONEFORMOTEROL:Vitamin supplementation:0.68), (BELATACEPT:Vitamin supplementation:0.48), (BENSERAZIDELEVODOPA:Vitamin supplementation:0.34), (BENZOYL PEROXIDECLINDAMYCIN PHOSPHATE:Vitamin supplementation:0.07), (BEPOTASTINE BESILATE:Vitamin supplementation:0.53), (BEPRIDIL:Vitamin supplementation:0.05), (BERAPROST SODIUM:Vitamin supplementation:1.36), (BETAHISTINE MESILATE:Vitamin supplementation:0.13), (BETAMETHASONE DIPROPIONATECALCIPOTRIENE HYDRATE:Vitamin supplementation:0.53), (BETAMETHASONE SODIUM PHOSPHATE:Vitamin supplementation:1.2), (BETAMETHASONECALCIPOTRIENE:Vitamin supplementation:1.78), (BETHANECHOL CHLORIDE:Vitamin supplementation:10.3), (BEXAROTENE:Vitamin supplementation:0.01), (BIPERIDEN HYDROCHLORIDE:Vitamin supplementation:0.4), (BLINATUMOMAB:Vitamin supplementation:1.09), (BOCEPREVIR:Vitamin supplementation:0.96), (BOSUTINIB:Vitamin supplementation:0.28), (BOTULINUM TOXIN TYPE A:Vitamin supplementation:0.84), (BRINZOLAMIDETIMOLOL MALEATE:Vitamin supplementation:0.49), (BRIVARACETAM:Vitamin supplementation:0.26), (BROMELAINS:Vitamin supplementation:0.05), (BROMHEXINE:Vitamin supplementation:0.33), (CABOZANTINIB:Vitamin supplementation:0.24), (CABOZANTINIB S-MALATE:Vitamin supplementation:0.8), (CALCIUM BICARBONATE:Vitamin supplementation:0.38), (CALCIUM CHLORIDEDEXTROSE MONOHYDRATEMAGNESIUM CHLORIDE\SODIUM CHLORIDE\SODIUM LACTATE:Vitamin supplementation:0.48), (CANAGLIFLOZINMETFORMIN HYDROCHLORIDE:Vitamin supplementation:2.01), (CANNABIDIOL:Vitamin supplementation:0.13), (CARBAZOCHROME SODIUM SULFONATE:Vitamin supplementation:0.16), (CASPOFUNGIN:Vitamin supplementation:0.78), (CEFDITOREN PIVOXIL:Vitamin supplementation:0.17), (CEFOTAXIMECEFOTAXIME SODIUM:Vitamin supplementation:0.81), (CEFPROZIL:Vitamin supplementation:0.95), (CEFTAZIDIME SODIUM:Vitamin supplementation:0.28), (CERTOPARIN SODIUM:Vitamin supplementation:0.62), (CHLORAMPHENICOL:Vitamin supplementation:0.43), (CHLORMADINONE ACETATE:Vitamin supplementation:0), (CHOLESTEROL:Vitamin supplementation:0.64), (CICLOPIROX OLAMINE:Vitamin supplementation:0.69), (CISATRACURIUM:Vitamin supplementation:0.13), (CLOMIPRAMINE:Vitamin supplementation:0.04), (CLOXACILLIN SODIUM:Vitamin supplementation:0.19), (CODEINE PHOSPHATEPROMETHAZINE:Vitamin supplementation:0.11), (COLISTIMETHATE SODIUM:Vitamin supplementation:0.99), (CORTISONE ACETATE:Vitamin supplementation:0.81), (CRANBERRY CONCENTRATE:Vitamin supplementation:0.01), (CROMOLYN SODIUM:Vitamin supplementation:0.04), (CROTALIDAE POLYVALENT IMMUNE FAB (OVINE):Vitamin supplementation:0.11), (CYAMEMAZINE:Vitamin supplementation:0.5), (DABRAFENIB MESYLATE:Vitamin supplementation:0.77), (DACLIZUMAB:Vitamin supplementation:2.72), (DEFERIPRONE:Vitamin supplementation:0.95), (DEFEROXAMINE MESYLATE:Vitamin supplementation:0.49), (DEXAMETHASONENEOMYCINPOLYMYXIN B SULFATE:Vitamin supplementation:0.08), (DEXTROAMPHETAMINE SULFATE:Vitamin supplementation:1.91), (DIAZOXIDE:Vitamin supplementation:0.06), (DIENOGEST:Vitamin supplementation:0.56), (DIHYDROCODEINE PHOSPHATE:Vitamin supplementation:0.08), (DIMETHINDENE:Vitamin supplementation:0.75), (DIPHTHERIA AND TETANUS TOXOIDS AND ACELLULAR PERTUSSIS VACCINE ADSORBED:Vitamin supplementation:0.18), (DOLASETRON MESYLATE:Vitamin supplementation:1.27), (DOPAMINE HYDROCHLORIDE:Vitamin supplementation:0.31), (DOXYCYCLINE HYDROCHLORIDE:Vitamin supplementation:0.08), (DOXYLAMINE SUCCINATE:Vitamin supplementation:1.2), (ELBASVIRGRAZOPREVIR:Vitamin supplementation:0.65), (EMTRICITABINE:Vitamin supplementation:0.75), (EMTRICITABINERILPIVIRINE HYDROCHLORIDETENOFOVIR DISOPROXIL FUMARATE:Vitamin supplementation:0.91), (EMTRICITABINERILPIVIRINETENOFOVIR DISOPROXIL FUMARATE:Vitamin supplementation:0.43), (ENFUVIRTIDE:Vitamin supplementation:0.06), (EPHEDRINE:Vitamin supplementation:0.03), (EPHEDRINE HYDROCHLORIDEGUAIFENESIN:Vitamin supplementation:0.3), (EPINASTINE HYDROCHLORIDE:Vitamin supplementation:1.22), (EPIRUBICIN:Vitamin supplementation:1.05), (ERIBULIN:Vitamin supplementation:0.69), (ESTRADIOL BENZOATE:Vitamin supplementation:0.1), (ESTRADIOLLEVONORGESTREL:Vitamin supplementation:0.43), (ETHACRYNIC ACID:Vitamin supplementation:1.2), (ETHINYL ESTRADIOLFERROUS FUMARATENORETHINDRONE:Vitamin supplementation:0.22), (ETHOSUXIMIDE:Vitamin supplementation:0.06), (ETOPOSIDE PHOSPHATE:Vitamin supplementation:0.54), (ETRETINATE:Vitamin supplementation:1.35), (EUCALYPTOLMENTHOLMETHYL SALICYLATETHYMOL:Vitamin supplementation:0.11), (FERRIC HYDROXIDE:Vitamin supplementation:0.17), (FERRIC POLYSACCHARIDE COMPLEX:Vitamin supplementation:0.3), (FERROUS GLYCINE SULFATE:Vitamin supplementation:0.46), (FESOTERODINE:Vitamin supplementation:0.03), (FEXOFENADINE HYDROCHLORIDEPSEUDOEPHEDRINE HYDROCHLORIDE:Vitamin supplementation:0.03), (FILGRASTIM-SNDZ:Vitamin supplementation:0.09), (FLUNARIZINE HYDROCHLORIDE:Vitamin supplementation:0.67), (FLUOCINOLONE ACETONIDE:Vitamin supplementation:1.5), (FLUOXETINE HYDROCHLORIDEOLANZAPINE:Vitamin supplementation:0.02), (FLURAZEPAMFLURAZEPAM HYDROCHLORIDE:Vitamin supplementation:0.73), (FLUTAMIDE:Vitamin supplementation:0.45), (FONDAPARINUX:Vitamin supplementation:1), (FONDAPARINUX SODIUM:Vitamin supplementation:1.17), (FORMOTEROL FUMARATE\MOMETASONE FUROATE:Vitamin supplementation:2.1), (GADOBUTROL:Vitamin supplementation:0.06), (GADOLINIUM:Vitamin supplementation:0.75), (GELATIN:Vitamin supplementation:0.14), (GLIPIZIDEMETFORMIN HYDROCHLORIDE:Vitamin supplementation:0.74), (GLYCOPYRROLATEINDACATEROL MALEATE:Vitamin supplementation:1.31), (HALCINONIDE:Vitamin supplementation:0.11), (HALOPERIDOL DECANOATE:Vitamin supplementation:0.09), (HIGH DENSITY POLYETHYLENE:Vitamin supplementation:0.24), (HOMATROPINE METHYLBROMIDEHYDROCODONE BITARTRATE:Vitamin supplementation:0.3), (IBRITUMOMAB TIUXETAN:Vitamin supplementation:0.58), (ICATIBANT ACETATE:Vitamin supplementation:0.35), (ICOSAPENT:Vitamin supplementation:0.23), (INDAPAMIDEPERINDOPRIL:Vitamin supplementation:2.52), (INFLUENZA VIRUS VACCINE TRIVALENT TYPE A+B AFLURIA:Vitamin supplementation:0.2), (INSULIN PORKINSULIN PURIFIED PORK:Vitamin supplementation:0.8), (INTERFERON:Vitamin supplementation:2.2), (INTERFERON ALFA-2A:Vitamin supplementation:1.76), (INTERFERON ALFA-2B:Vitamin supplementation:1.41), (INTERFERON GAMMA-1B:Vitamin supplementation:1.46), (ISONIAZIDPYRAZINAMIDERIFAMPIN:Vitamin supplementation:0.33), (LEVALBUTEROL TARTRATE:Vitamin supplementation:0.9), (LEVOBUNOLOL HYDROCHLORIDE:Vitamin supplementation:0.23), (LEVOLEUCOVORIN CALCIUM:Vitamin supplementation:0.25), (LEVOTHYROXINE SODIUMPOTASSIUM IODIDE:Vitamin supplementation:1.17), (LINSIDOMINE:Vitamin supplementation:0.16), (LOMUSTINE:Vitamin supplementation:0.34), (LUTETIUM LU-177:Vitamin supplementation:0.67), (MAGNESIUMPYRIDOXINE:Vitamin supplementation:0.28), (MARAVIROC:Vitamin supplementation:0.86), (MAXACALCITOL:Vitamin supplementation:0.33), (MECLOZINE DIHYDROCHLORIDE:Vitamin supplementation:0.06), (MELPERONE:Vitamin supplementation:0.28), (MENTHOLZINC OXIDE:Vitamin supplementation:0.47), (MEPOLIZUMAB:Vitamin supplementation:1.95), (MEPREDNISONE:Vitamin supplementation:0.15), (MESTRANOLNORETHINDRONE:Vitamin supplementation:0.25), (METFORMIN HYDROCHLORIDEROSIGLITAZONE MALEATE:Vitamin supplementation:0.08), (METFORMIN HYDROCHLORIDESAXAGLIPTIN:Vitamin supplementation:0.42), (METFORMIN PAMOATE:Vitamin supplementation:0.46), (METHENAMINE MANDELATE:Vitamin supplementation:0.85), (METRELEPTIN:Vitamin supplementation:0.83), (MIGLITOL:Vitamin supplementation:0.09), (MITOXANTRONE HYDROCHLORIDE:Vitamin supplementation:0.51), (NABILONE:Vitamin supplementation:0.35), (NAFTIFINE HYDROCHLORIDE:Vitamin supplementation:0.01), (NALBUPHINE HYDROCHLORIDE:Vitamin supplementation:0.27), (NALOXONE HYDROCHLORIDETILIDINE HYDROCHLORIDE:Vitamin supplementation:2.49), (NALTREXONE HYDROCHLORIDE:Vitamin supplementation:0.22), (NAPHAZOLINE HYDROCHLORIDE:Vitamin supplementation:0.55), (NEFAZODONE:Vitamin supplementation:0.3), (NEFAZODONE HYDROCHLORIDE:Vitamin supplementation:0.41), (NISOLDIPINE:Vitamin supplementation:0.53), (NOREPINEPHRINE:Vitamin supplementation:0.89), (NOREPINEPHRINE BITARTRATE:Vitamin supplementation:1.4), (NUSINERSEN:Vitamin supplementation:0.45), (OCRELIZUMAB:Vitamin supplementation:0.1), (OLODATEROL HYDROCHLORIDE:Vitamin supplementation:0.33), (OPIPRAMOL:Vitamin supplementation:1.08), (OSELTAMIVIR:Vitamin supplementation:1.46), (OXYBUTYNINOXYBUTYNIN CHLORIDE:Vitamin supplementation:1.16), (PANITUMUMAB:Vitamin supplementation:0.54), (PAPAYA:Vitamin supplementation:0.37), (PAROXETINE MESYLATE:Vitamin supplementation:0.09), (PEGZEREPOETIN ALFA:Vitamin supplementation:0.13), (PENICILLAMINE:Vitamin supplementation:0.24), (PENICILLIN V BENZATHINE:Vitamin supplementation:0.15), (PENTOSTATIN:Vitamin supplementation:0.97), (PERINDOPRIL ARGININE:Vitamin supplementation:0.02), (PERTUZUMAB:Vitamin supplementation:0.78), (PHENOBARBITAL SODIUM:Vitamin supplementation:0.65), (PHENTERMINE HYDROCHLORIDE:Vitamin supplementation:0.15), (PILSICAINIDE HYDROCHLORIDE:Vitamin supplementation:0.31), (PIRITRAMIDE:Vitamin supplementation:0.39), (POTASSIUM CITRATETRISODIUM CITRATE DIHYDRATE:Vitamin supplementation:0.2), (POTASSIUM IODIDE:Vitamin supplementation:0.54), (POTASSIUM PHOSPHATE, MONOBASIC:Vitamin supplementation:0.43), (PRAZEPAM:Vitamin supplementation:0.33), (PREDNISOLONE ACETATESULFACETAMIDE SODIUM:Vitamin supplementation:0.43), (PREDNISOLONE SODIUM SUCCINATE:Vitamin supplementation:0.45), (PREGNENOLONE:Vitamin supplementation:0.41), (PRISTINAMYCIN:Vitamin supplementation:0.69), (PROCARBAZINE HYDROCHLORIDE:Vitamin supplementation:0.02), (PSEUDOEPHEDRINE:Vitamin supplementation:0.35), (PSORALEN:Vitamin supplementation:0.02), (QUINACRINE:Vitamin supplementation:1.62), (RAMUCIRUMAB:Vitamin supplementation:1), (RIOCIGUAT:Vitamin supplementation:0.27), (ROPIVACAINE HYDROCHLORIDE:Vitamin supplementation:0.1), (RUCAPARIB CAMSYLATE:Vitamin supplementation:0.4), (RUFINAMIDE:Vitamin supplementation:0.39), (RUPATADINE FUMARATE:Vitamin supplementation:0.11), (SALSALATE:Vitamin supplementation:0.05), (SELEXIPAG:Vitamin supplementation:0.26), (SERRAPEPTASE:Vitamin supplementation:0.59), (SEVELAMER:Vitamin supplementation:0.78), (SEVOFLURANE:Vitamin supplementation:0.06), (SIBUTRAMINE HYDROCHLORIDE:Vitamin supplementation:0.94), (SILICON DIOXIDE:Vitamin supplementation:0.26), (SODIUM CITRATE:Vitamin supplementation:0.75), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Vitamin supplementation:0.01), (SODIUM PHOSPHATE, DIBASIC\SODIUM PHOSPHATE, MONOBASIC:Vitamin supplementation:2.16), (STREPTOMYCIN\STREPTOMYCIN SULFATE:Vitamin supplementation:0.3), (SUFENTANIL:Vitamin supplementation:0.44), (SUFENTANIL CITRATE:Vitamin supplementation:1.12), (SULFACETAMIDE SODIUMSULFUR:Vitamin supplementation:0.26), (SUPLATAST TOSILATE:Vitamin supplementation:0.42), (TEDUGLUTIDE:Vitamin supplementation:0.93), (TEGASEROD MALEATE:Vitamin supplementation:0.35), (TEMSIROLIMUS:Vitamin supplementation:0.5), (TERCONAZOLE:Vitamin supplementation:0.71), (TESTOSTERONE UNDECANOATE:Vitamin supplementation:1.73), (TETRACAINE HYDROCHLORIDE:Vitamin supplementation:0.07), (THEOPHYLLINE:Vitamin supplementation:1.26), (THIOPENTAL:Vitamin supplementation:0.31), (TIAGABINE HYDROCHLORIDE:Vitamin supplementation:0.1), (TICLOPIDINE:Vitamin supplementation:0.44), (TILIDINE:Vitamin supplementation:0.55), (TIMOLOL MALEATETRAVOPROST:Vitamin supplementation:1.25), (TRAMETINIB DIMETHYL SULFOXIDE:Vitamin supplementation:0.75), (TRIPTORELIN PAMOATE:Vitamin supplementation:0.97), (UREA:Vitamin supplementation:0.07), (VALDECOXIB:Vitamin supplementation:1.04), (VELAGLUCERASE ALFA:Vitamin supplementation:0.1), (VINORELBINEVINORELBINE TARTRATE:Vitamin supplementation:0.83), (VISMODEGIB:Vitamin supplementation:0.78), (YEAST:Vitamin supplementation:1.2), (ZICONOTIDE ACETATE:Vitamin supplementation:1.18), (ZILEUTON:Vitamin supplementation:1.39), (ZINC ACETATE:Vitamin supplementation:0.14), (ZIV-AFLIBERCEPT:Vitamin supplementation:0.06), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:Vomiting:0.65), (.ALPHA.-TOCOPHEROLVITAMINS:Vomiting:0.15), (.ALPHA.1-PROTEINASE INHIBITOR HUMAN:Vomiting:1.71), (ABOBOTULINUMTOXINA:Vomiting:0.51), (ACETAMINOPHEN CAFFEINE:Vomiting:0.88), (ACETAMINOPHEN\CAFFEINEOPIUM:Vomiting:0.76), (ADEN OSINE:Vomiting:0.12), (AJMALINE:Vomiting:0.06), (ALECTINIB:Vomiting:0.2), (ALFUZOSI N HYDROCHLORIDE:Vomiting:1.49), (ALUMINUM HYDROXIDEMAGNESIUM OXIDEOXETHAZAINE:Vomiting:0.66), (AMISULPRIDE:Vomiting:1.55), (AMLODIPINE BESYLATEIRBESARTAN:Vomiting:0.14), (AMLODIPINEPERINDOPRIL:Vomiting:0.95), (AMLODIPINEVALSARTAN:Vomiting:1.62), (AMPICILLIN SODIUM:Vomiting:0.67), (ANAGRELIDE:Vomiting:0.31), (ANAGRELIDE HYDROCHLORIDE:Vomiting:0.67), (ANHYDROUS CITRIC ACIDASPIRIN\SODIUM BICARBONATE:Vomiting:0.37), (ANIDULAFUNGIN:Vomiting:0.94), (ANTIHEMOPHILIC FACTOR, HUMAN RECOMBINANT:Vomiting:0.52), (ARIPIPRAZOLE LAUROXIL:Vomiting:0.16), (ASENAPINE MALEATE:Vomiting:0.08), (ASPIRINCLOPIDOGREL:Vomiting:0.42), (ASPIRINDIHYDRO XYALUMINUM AMINOACETATE ANHYDROUSMAGNESIUM CARBONATE:Vomiting:0.42), (ASPIRINMAGNESIUM OXIDE:Vomiting:0.89), (ATAZANAVIRRITONAVIR:Vomiting:0.08), (ATRACURIUM:Vomi ting:0.32), (ATRACURIUM BESYLATE:Vomiting:0.51), (AZITHROMYCIN OR METHOTREXATE SODIUM:Vomiting:0.12), (BAMIFYLLINE HYDROCHLORIDE:Vomiting:0.01), (BECLOMETHASONE DIPROPIONATEFORMOTEROL:Vomiting:0.31), (BELATACEPT:Vomiting:0.91), (BERAPR OST:Vomiting:0.19), (BERAPROST SODIUM:Vomiting:1), (BETAHISTINE MESILATE:Vomiting:0), (BETAMETHASONE DIPROPIONATECALCIPOTRIENE:Vomiting:0.54), (BETAMETHASONE DIPROPIONATECALCIPOTRIENECALCIPOTRIENE HYDRATE:Vomiting:0.94), (BETAMETHASONECALCIPOTRIENE:Vomiting:1.12), (BETH ANECHOL CHLORIDE:Vomiting:0.78), (BILASTINE:Vomiting:0.77), (BISACODYLGLYCERINMINER AL OILSODIUM PHOSPHATE, DIBASIC\SODIUM PHOSPHATE, MONOBASIC:Vomiting:0.42), (BLESELUMAB:Vomiting:0.18), (BOCOCIZUMAB:Vomiting: 0.21), (BOTULINUM TOXIN TYPE A:Vomiting:0.39), (BRINZOLAMIDETIMOLOL MALEATE:Vomiting:0.09), (BUDESONIDEFORMOTEROL FUMARATE:Vomiting:1.43), (CABOZANTINIB:Vomiting:0.61), (CALCIUM PANTOTHENATECYANOCOBALAMINNIACINAMIDEPYRIDOXINE HYDROCHLORIDE\RIBOFLAVINTHIAMINE MONONITRATE:Vomiting:0.45), (CALCIUM\MAGNESIUM\ZINC:Vomiting:0.99), (CARBID OPA:Vomiting:0.94), (CARBOMER:Vomiting:0.05), (CARNITINE:Vomiting:0.32), (CEFOTAX IMECEFOTAXIME SODIUM:Vomiting:1.59), (CEFOXITINCEFOXITIN SODIUM:Vomiting:0.09), (CEFPROZIL:Vomiting:0.94), (CHLORMADINONE ACETATE:Vomiting:0.42), (CHONDROITIN SULFATE (CHICKEN):Vomiting:0.68), (CISATRACURIUM:Vomiting:0.82), (CLODRONATE DISODIUM:Vomiting:0.48), (CLOXACILLIN SODIUM:Vomiting:0.32), (CLOXAZOLAM:Vomiting:0.12), (COAGULATION FACTOR IX HUMANCOAGULATION FACTOR VII HUMANCOAGULATION FACTOR X HUMANPROTEIN CPROTEIN S HUMANPROTHROMBIN:Vomiting:0.47), (COBICISTATELVITEGRAVIREMTRICITABI NETENOFOVIR ALAFENAMIDE FUMARATE:Vomiting:0.95), (COLCHICINEDICYCLOMINE HYDROCHLORIDE:Vomiting:0.1), (CONVALLARIA MAJALISCRATAEGUS LAEVIGATA LEAFPROXYPHYLLINE:Vomiting:0.09), (CORTISONE ACETATE:Vomiting:0.39), (CURCUMIN:Vomiting:0.35), (CYCLOPENTOLATE:Vomiting:0.42), (CYCLOPHOSPHAMIDECYTARABINEDEXAMETHASONEDOXORUBICINMETHO TREXATEVINCRISTINE:Vomiting:0.47), (CYPROTERONE ACETATE:Vomiting:0.02), (DABIGATRAN:Vomiting:1.47), (DACLIZUMAB:Vomiting:2.35), (DANTROLENE SODIUM:Vomiting:1.09), (DARUNAVIRRITONAVIR:Vomiting:0.16), (DESOGESTRELET HINYL ESTRADIOL:Vomiting:0.18), (DEXMETHYLPHENIDATE HYDROCHLORIDE:Vomiting:0.14), (DEXPANTHENOLNIACINAMIDEPYRIDOXINE HYDROCHLORIDE\RIBOFLAVIN 5′-PHOSPHATE SODIUMTHIAMINE HYDROCHLORIDE:Vomiting:0.27), (DEXTROAMPHETAMINE SULFATE:Vomiting:1.09), (DICLOFENAC SODIUMMISOPROSTOL:Vomiting:0.48), (DIOSMIN:Vomiting:0.13), (DOBUTAMINE HYDROCHLORIDE:Vomiting:1.1), (DORIPENEM:Vomiting:0.29), (DUTASTERIDETAMSU LOSIN HYDROCHLORIDE:Vomiting:0.97), (ELBASVIRGRAZOPREVIR:Vomiting:0.72), (EMPAG LIFLOZINLINAGLIPTIN:Vomiting:1.2), (EMTRICITABINERILPIVIRINE HYDROCHLORIDETENOFOVIR DISOPROXIL FUMARATE:Vomiting:0.4), (EMTRICITABINERILPIVIRINETENOFOVIR DISOPROXIL FUMARATE:Vomiting:0.58), (ENALAPRIL MALEATELERCANIDIPINE HYDROCHLORIDE:Vomiting:0.9), (EPACADOSTAT:Vomiting:0.09), (EPHEDRINE HYDROCHLORIDE:Vomiting:0.57), (ESMOLOL HYDROCHLORIDE:Vomiting:1.32), (ESTROGENS, ESTERIFIEDMETHYLTESTOSTERONE:Vomiting:0.03), (ETHACRYNIC ACID:Vomiting:0.62), (ETHIODIZED OIL:Vomiting:0.29), (ETIMICIN SULFATE:Vomiting:0.21), (ETRETINATE:Vomiting:0.93), (EUCALYPTOLMENTHOLMET HYL SALICYLATETHYMOL:Vomiting:0.09), (FENOTEROL HYDROBROMIDEIPRATROPIUM BROMIDE:Vomiting:1.01), (FERROUS BISGLYCINATE:Vomiting:0.07), (FERROUS CITRATE:Vomiting:0.51), (FERROUS GLYCINE SULFATE:Vomiting:0.16), (FERROUS SULFATEGLYCINE:Vomiting:1.31), (FIBRINOGEN HUMAN:Vomiting:0.78), (FISH OILTOCOPHEROL:Vomiting:0.03), (FLUCYTOSINE:Vomiting:0.71), (FLURAZEPAMFLU RAZEPAM HYDROCHLORIDE:Vomiting:0.75), (FLUTAMIDE:Vomiting:0.56), (FOLIC ACIDIRONMINERALSVITAMINS:Vomiting:0.58), (FORMOTEROL FUMARATE\MOMETASONE FUROATE:Vomiting:1.21), (FOSFOMYCIN:Vomiting:1.06), (FOSFOMYCIN SODIUM:Vomiting:0.06), (FOSPHENYTOIN SODIUM:Vomiting:0.28), (FROVATRIPTAN SUCCINATE:Vomiting:0.1), (GADOBENATE DIMEGLUMINE:Vomiting:0.24), (GADOLINIUM:Vomiting:0.82), (GLIPIZIDEMETFORMIN HYDROCHLORIDE:Vomiting:0.29), (GLYCOPYRROLATEINDACATEROL MALEATE:Vomiting:0.8), (GLYCOPYRRONIUMINDACATEROL:Vomiting:2), (GUANFAC INE:Vomiting:1.19), (GUANFACINE HYDROCHLORIDE:Vomiting:0.95), (HYDROCODONE BITARTRATEIBUPROFEN:Vomiting:1.01), (HYDROCORTISONE HEMISUCCINATE:Vomiting:0.08), (HYOSCYAMINE SULFATEMETHENAMINEMETHYLENE BLUEPHENYL SALICYLATE\SODIUM PHOSPHATE, MONOBASIC, MONOHYDRATE:Vomiting:0.17), (IDARUCIZUMAB:Vomiting:0.4), (ILOPROST:Vomiting:10.55), (IMIPRAMINE HYDROCHLORIDE:Vomiting:2.06), (INFLIXIMAB-DYYB:Vomiting:0.49), (INTERFERON ALFA-2A OR INTERFERON ALFA-2B:Vomiting:0.49), (IRON POLYMALTOSE:Vomiting:0.37), (ISOFLURANE:Vomiting:0.5), (ISONIAZIDPYRAZINAMI DERIFAMPIN:Vomiting:0.25), (ISONIAZIDRIFAMPIN:Vomiting:0.63), (ITRACONAZOLE SODIUM CHLORIDE:Vomiting:0.16), (IVABRADINE HYDROCHLORIDE:Vomiting:0.4), (LACIDIPINE:Vomiting:0.03), (LACTITOL:Vomiting:0.29), (LANREOTIDE:Vomiting:0.58), (LANTHANUM CARBONATE:Vomiting:0.05), (LEVOBUPIVACAINE:Vomiting:0.06), (LEVOSIMENDAN:V omiting:0.07), (LEVOTHYROXINE SODIUMPOTASSIUM IODIDE:Vomiting:1.11), (LIMAPROST:Vomiting:0.24), (LINSIDOMINE:Vomiting:0.38), (LU MACAFTOR:Vomiting:0.11), (LUTETIUM LU-177:Vomiting:0.36), (MAGNESIUM ASPARTATE\POTASSIUM ASPARTATE:Vomiting:0.49), (MEPOLIZUMAB:Vomiting:0.97), (METFORMIN HYDROCHLORIDESAXAGLIPTIN:Vomiting:0.38), (METFORMIN PAMOATE:Vomiting:0.3), (METFORMINSITAGLIPTIN:Vomiting:0.1), (METHENAMINE MANDELATE:Vomiting:0.12), (METHENOLONE ENANTHATE:Vomiting:1.03), (METILDIGOXIN:Vomiting:1.02), (MIGLUSTAT:Vomiting:0.81), (MIZORIBINE:Vomiting:0.5), (MORPHINE\MORPHINE HYDROCHLORIDE\MORPHINE SULFATE\MORPHINE TARTRATE:Vomiting:0.04), (NATEGLINIDE:Vomiting:1.34), (NELARABINE:Vomiting:0.26), (NEOSTIGMINE:Vomiting:0.96), (NUSINERSEN:Vomiting:0.23), (OMACETAXINE MEPESUCCINATE:Vomiting:0.07), (OXYBUTYNINOXYBUTYNIN CHLORIDE:Vomiting:0.18), (PALIPERIDONE PALMITATE:Vomiting:0.41), (PANTOPRAZOLE MAGNESIUM:Vomiting:2.5), (PASIREOTIDE:Vomiting:0.76), (PEGINTERFERON ALFA-2A OR PEGINTERFERON ALFA-2B OR PEGINTERFERON BETA-1A OR PEGINTERFERON LAMBDA-1A:Vomiting:0.66), (PEGZEREPOETIN ALFA:Vomiting:0.1), (PENICILLIN V BENZATHINE:Vomiting:0.58), (PENTOSTATIN:Vomiting:2.09), (PERAMPANEL:Vomiting:00.16), (PERPHENAZINE:Vomiting:0.14), (PHENAZOPYRIDINE HYDROCHLORIDE:Vomiting:0.82), (PHOSPHOCREATINE SODIUM:Vomiting:0.09), (PILOCARPINE:Vomiting:1.2), (PILSICAINIDE HYDROCHLORIDE:Vomiting:0.37), (PINDOLOL:Vomiting:0.04), (PIRBUTEROL ACETATE:Vomiting:0.2), (POLYMYXIN:Vomiting:0.23), (POTASSIUM CITRATETRISODIUM CITRATE DIHYDRATE:Vomiting:0.2), (POVIDONE:Vomiting:0.04), (PRAMOXINE HYDROCHLORIDE:Vomiting:0.1), (PRANLUKAST:Vomiting:0.37), (PRASTERONE:Vomitin g:0.45), (PROTRIPTYLINE HYDROCHLORIDE:Vomiting:0.44), (PYRIMETHAMINE:Vomiting:0.52), (QUINACRINE:Vo miting:0.64), (RILMENIDINE PHOSPHATE:Vomiting:0.93), (RILPIVIRINE HYDROCHLORIDE:Vomiting:0.47), (RIOCIGUAT:Vomiting:0.27), (ROFECOXIB:Vomiting:00.46), (RUPATADINE FUMARATE:Vomiting:0.11), (SAMARIUM SM-153 LEXIDRONAM PENTASODIUM:Vomiting:0.03), (SAPROPTERIN DIHYDROCHLORIDE:Vomiting:0.33), (SELEXIPAG:Vomiting:0.13), (SEVOFLURANE:Vom iting:0.81), (SIBUTRAMINE HYDROCHLORIDE:Vomiting:0.12), (SILICON DIOXIDE:Vomiting:0.38), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Vomiting:0.46), (SOFOSBUVIRVELPATASVIR:Vomiting:0.6), (SOMAT OSTATIN:Vomiting:0.27), (SOTALOL HYDROCHLORIDE:Vomiting:0.81), (SPIRAMYCIN:Vomiting:0.43), (STREPTOMYCINSTR EPTOMYCIN SULFATE:Vomiting:1.05), (TAFAMIDIS MEGLUMINE:Vomiting:0.02), (TASIMELTEON:Vomiting:0.12), (TEDUGLUTIDE:Vomiting: 1.18), (TEGAFURURACIL:Vomiting:0.2), (TELOTRISTAT ETHYL:Vomiting:1.11), (TESTOSTERONE UNDECANOATE:Vomiting:1.32), (TETRABENAZINE:Vomiting:0.14), (THEOPHYLLINE:Vo miting:0.92), (THIAMYLAL SODIUM:Vomiting:0.31), (THIOPENTAL:Vomiting:0.63), (THIOPENTAL SODIUM:Vomiting:0.08), (THROMBOMODULIN ALFA:Vomiting:0.13), (TICLOPIDINE:Vomiting:0.24), (TILIDINE:Vomiting:0.42), (TRIHEXY PHENIDYL:Vomiting:0.48), (TRIHEXYPHENIDYL HYDROCHLORIDE:Vomiting:0.1), (TRIMIPRAMINE MALEATE:Vomiting:1.1), (TRIPTORELIN PAMOATE:Vomiting:0.48), (TROSPIUM:Vomiting:0.1), (UMECLIDINIUM BROMIDE:Vomiting:1.26), (VALDECOXIB:Vomiting:0.31), (VANDETANIB:Vomiting:0.64), (VONOPRAZAN FUMURATE:Vomiting:0.01), (WARFARIN POTASSIUM:Vomiting:1.07), (YEAST:Vomiting:0.97), (ZILEUTON:Vomiting:0.72), (ACETA MINOPHENOXYCODONEOXYCODONE HYDROCHLORIDE:Vulvovaginal dryness:0.16), (ACETAZOLAMIDE:Vulvovaginal dryness:0.06), (AMIODARONE:Vulvovaginal dryness:0.43), (ATROPINE SULFATEDIPHENOXYLATE HYDROCHLORIDE:Vulvovaginal dryness:0.52), (AZITHROMYCIN DIHYDRATE:Vulvovaginal dryness:0.14), (BISOPROLOL FUMARATE:Vulvovaginal dryness:0.23), (BORTEZOMIB:Vulvovaginal dryness:0.14), (BUMETANIDE:Vulvovaginal dryness:0.37), (BUPRENORPHINE HYDROCHLORIDENALOXONE HYDROCHLORIDE:Vulvovaginal dryness:0.35), (CALCITRIOL:Vulvovaginal dryness:0.51), (CALCIUM CARBONATEVITAMIN D:Vulvovaginal dryness:0.37), (CANDESARTAN:Vulvovaginal dryness:0.28), (CANDESARTAN CILEXETILHYDROCHLOROTHIAZIDE:Vulvovaginal dryness:0.19), (CARFILZOMIB:Vulvovaginal dryness:0.18), (CARVEDILOL PHOSPHATE:Vulvovaginal dryness:0.14), (CERTOLIZUMAB PEGOL:Vulvovaginal dryness:1.08), (CETIRIZINE HYDROCHLORIDEPSEUDOEPHEDRINE HYDROCHLORIDE:Vulvovaginal dryness:0.25), (COD LIVER OIL:Vulvovaginal dryness:0.26), (DACLIZUMAB:Vulvovaginal dryness:0.44), (DAPAGLIFLOZIN PROPANEDIOL:Vulvovaginal dryness:0.16), (DASATINIB:Vulvovaginal dryness:0.46), (DEVICE:Vulvovaginal dryness:0.09), (DOFETILIDE:Vulvovaginal dryness:0.04), (DOXEPIN HYDROCHLORIDE:Vulvovaginal dryness:0.14), (DUTASTERIDE:Vulvovaginal dryness:0.3), (ENALAPRIL MALEATE:Vulvovaginal dryness:0.38), (ERYTHROMYCIN:Vulvovaginal dryness:0.13), (ESZOPICLONE:Vulvovaginal dryness:0.9), (ETHINYL ESTRADIOLNORGESTIMATE:Vulvovaginal dryness:0.16), (FERROUS SULFATEFERROUS SULFATE, DRIED:Vulvovaginal dryness:1.1), (FISH OIL\OMEGA-3 FATTY ACIDS:Vulvovaginal dryness:0.26), (FLUOXETINEFLUOXETINE HYDROCHLORIDE:Vulvovaginal dryness:0.47), (GLYBURIDE:Vulvovaginal dryness:0.1), (HERBALSTURMERIC:Vulvovaginal dryness:0.47), (HYDRALAZINE HYDROCHLORIDE:Vulvovaginal dryness:0.17), (HYDROXYCHLOROQUINE DIPHOSPHATE:Vulvovaginal dryness:0.48), (HYOSCYAMINE:Vulvovaginal dryness:0.32), (IMIPRAMINE HYDROCHLORIDE:Vulvovaginal dryness:0.2), (INDOMETHACIN:Vulvovaginal dryness:0.48), (IPRATROPIUM:Vulvovaginal dryness:0.04), (ISOSORBIDE:Vulvovaginal dryness:0.25), (LETROZOLE:Vulvovaginal dryness:0.21), (LINAGLIPTIN:Vulvovaginal dryness:0.12), (LISDEXAMFETAMINE DIMESYLATE:Vulvovaginal dryness:0.53), (METHADONE HYDROCHLORIDE:Vulvovaginal dryness:0.2), (MOXIFLOXACIN HYDROCHLORIDE:Vulvovaginal dryness:1.09), (NILOTINIB:Vulvovaginal dryness:0.35), (NORETHINDRONE:Vulvovaginal dryness:0.35), (OLANZAPINE:Vulvovaginal dryness:0.14), (PANTOPRAZOLE MAGNESIUM:Vulvovaginal dryness:0.45), (PAZOPANIB HYDROCHLORIDE:Vulvovaginal dryness:0.05), (PEGINTERFERON BETA-1A:Vulvovaginal dryness:0.4), (PENTOSAN POLYSULFATE SODIUM:Vulvovaginal dryness:0.2), (PONATINIB:Vulvovaginal dryness:0.23), (PRAMIPEXOLE:Vulvovaginal dryness:0.07), (PYRIDOXINE:Vulvovaginal dryness:0.09), (ROFLUMILAST:Vulvovaginal dryness:0.04), (SACUBITRILVALSARTAN:Vulvovaginal dryness:0.17), (SECUKINUMAB:Vulvovaginal dryness:0.22), (SOTALOL:Vulvovaginal dryness:0.24), (TAMSULOSIN HYDROCHLORIDE:Vulvovaginal dryness:0.77), (TERIFLUNOMIDE:Vulvovaginal dryness:0.9), (THALIDOMIDE:Vulvovaginal dryness:0.14), (UMECLIDINIUM BROMIDEVILANTEROL TRIFENATATE:Vulvovaginal dryness:0.07), (VARENICLINE TARTRATE:Vulvovaginal dryness:0.93), (VITAMIN A:Vulvovaginal dryness:0.01), (CALCIUM CARBONATECHOLECALCIFEROL:Vulvovaginal mycotic infection:0.04), (CYCLOBENZAPRINE:Vulvovaginal mycotic infection:0.04), (DOCUSATE SODIUM:Vulvovaginal mycotic infection:0.08), (EVOLOCUMAB:Vulvovaginal mycotic infection:0.07), (INDOMETHACIN:Vulvovaginal mycotic infection:0.01), (MELATONIN:Vulvovaginal mycotic infection:0.12), (TERIFLUNOMIDE:Vulvovaginal mycotic infection:0.58), (VARENICLINE TARTRATE:Vulvovaginal mycotic infection:0.11), (ACENOCOUMAROL:Waldenstrom's macroglobulinaemia:0.02), (ACETAMINOPHENOXYCODONEOXYCODONE HYDROCHLORIDE:Waldenstrom's macroglobulinaemia:0.06), (AMPHETAMINE ASPARTATEAMPHETAMINE SULFATEDEXTROAMPHETAMINE SACCHARATEDEXTROAMPHETAMINE SULFATE:Waldenstrom's macroglobulinaemia:0.98), (CANDESARTAN CILEXETIL:Waldenstrom's macroglobulinaemia:0.93), (CANDESARTAN CILEXETILHYDROCHLOROTHIAZIDE:Waldenstrom's macroglobulinaemia:0.82), (CARVEDILOL PHOSPHATE:Waldenstrom's macroglobulinaemia:0.4), (CERTOLIZUMAB PEGOL:Waldenstrom's macroglobulinaemia:0.66), (CHLORTHALIDONE:Waldenstrom's macroglobulinaemia:0.41), (CLINDAMYCIN:Waldenstrom's macroglobulinaemia:0.06), (COD LIVER OIL:Waldenstrom's macroglobulinaemia:0.05), (COLESEVELAM HYDROCHLORIDE:Waldenstrom's macroglobulinaemia:0.11), (DACLIZUMAB:Waldenstrom's macroglobulinaemia:0.13), (DANAZOL:Waldenstrom's macroglobulinaemia:0.28), (DASATINIB:Waldenstrom's macroglobulinaemia:0.94), (DICLOFENAC SODIUM:Waldenstrom's macroglobulinaemia:0.86), (DOFETILIDE:Waldenstrom's macroglobulinaemia:0.3), (ESTROGENS, CONJUGATED:Waldenstrom's macroglobulinaemia:0.87), (EVOLOCUMAB:Waldenstrom's macroglobulinaemia:0.61), (EZETIMIBESIMVASTATIN:Waldenstrom's macroglobulinaemia:0.33), (FESOTERODINE FUMARATE:Waldenstrom's macroglobulinaemia:0.6), (FLUOXETINEFLUOXETINE HYDROCHLORIDE:Waldenstrom's macroglobulinaemia:0.33), (FLUTICASONE:Waldenstrom's macroglobulinaemia:0.52), (FLUTICASONE FUROATE:Waldenstrom's macroglobulinaemia:0.01), (GLYCOPYRRONIUMINDACATEROL:Waldenstrom's macroglobulinaemia:0.05), (ILOPROST:Waldenstrom's macroglobulinaemia:0.07), (IMATINIB MESYLATE:Waldenstrom's macroglobulinaemia:0.03), (IMIPRAMINE HYDROCHLORIDE:Waldenstrom's macroglobulinaemia:0.11), (INDOMETHACIN:Waldenstrom's macroglobulinaemia:0.2), (INFLUENZA VIRUS VACCINE:Waldenstrom's macroglobulinaemia:0.01), (INSULIN DETEMIR:Waldenstrom's macroglobulinaemia:0.32), (LABETALOLLABETALOL HYDROCHLORIDE:Waldenstrom's macroglobulinaemia:0.44), (LEDIPASVIRSOFOSBUVIR:Waldenstrom's macroglobulinaemia:0.1), (LEVALBUTEROL HYDROCHLORIDE:Waldenstrom's macroglobulinaemia:0.72), (LIDOCAINEPRILOCAINE:Waldenstrom's macroglobulinaemia:0.28), (LISDEXAMFETAMINE DIMESYLATE:Waldenstrom's macroglobulinaemia:0.37), (LITHIUM CARBONATE:Waldenstrom's macroglobulinaemia:0.1), (METHENAMINE HIPPURATE:Waldenstrom's macroglobulinaemia:0.23), (METHYLPREDNISOLONE ACETATE:Waldenstrom's macroglobulinaemia:0.6), (MYCOPHENOLATE MOFETILMYCOPHENOLATE MOFETIL HYDROCHLORIDE:Waldenstrom's macroglobulinaemia:0.06), (NALOXONE HYDROCHLORIDEOXYCODONE HYDROCHLORIDE:Waldenstrom's macroglobulinaemia:0.38), (NILOTINIB:Waldenstrom's macroglobulinaemia:0.72), (OLANZAPINE:Waldenstrom's macroglobulinaemia:0.51), (OLODATEROL HYDROCHLORIDETIOTROPIUM BROMIDE MONOHYDRATE:Waldenstrom's macroglobulinaemia:0.01), (PENICILLIN:Waldenstrom's macroglobulinaemia:0.71), (PONATINIB:Waldenstrom's macroglobulinaemia:0.86), (QUETIAPINE FUMARATE:Waldenstrom's macroglobulinaemia:0.49), (ROFLUMILAST:Waldenstrom's macroglobulinaemia:0.04), (SACUBITRILVALSARTAN:Waldenstrom's macroglobulinaemia:0.73), (SODIUM OXYBATE:Waldenstrom's macroglobulinaemia:1.01), (SULFAMETHOXAZOLE:Waldenstrom's macroglobulinaemia:0.18), (SUMATRIPTANSUMATRIPTAN SUCCINATE:Waldenstrom's macroglobulinaemia:0.73), (TERIFLUNOMIDE:Waldenstrom's macroglobulinaemia:0.14), (UMECLIDINIUM BROMIDEVILANTEROL TRIFENATATE:Waldenstrom's macroglobulinaemia:0.26), (ZINC:Waldenstrom's macroglobulinaemia:0.22), (ACETAMINOPHENBUTALBITALCAFFEINE:Weight control:0.35), (AMISULPRIDE:Weight control:0.03), (ANASTROZOLE:Weight control:0.41), (ATROPINE SULFATEDIPHENOXYLATE HYDROCHLORIDE:Weight control:0.12), (AZITHROMYCIN DIHYDRATE:Weight control:0.18), (CALCIUM ACETATE:Weight control:0.49), (CALCIUM CARBONATECHOLECALCIFEROL:Weight control:0.64), (CANAGLIFLOZINMETFORMIN HYDROCHLORIDE:Weight control:0.28), (CANDESARTAN CILEXETILHYDROCHLOROTHIAZIDE:Weight control:0.4), (CAPTOPRIL:Weight control:0.1), (CERTOLIZUMAB PEGOL:Weight control:0.91), (CHOLESTYRAMINE:Weight control:0.28), (CLOTRIMAZOLE:Weight control:0.18), (COLESEVELAM HYDROCHLORIDE:Weight control:0.1), (DABIGATRAN:Weight control:0.03), (DACLIZUMAB:Weight control:0.08), (DASATINIB:Weight control:0.25), (DESLORATADINE:Weight control:0.26), (DOCUSATE CALCIUM:Weight control:0.16), (EDOXABAN TOSYLATE:Weight control:0.07), (ELETRIPTAN HYDROBROMIDE:Weight control:0.53), (EMPAGLIFLOZINLINAGLIPTIN:Weight control:0.51), (ENOXAPARIN SODIUM:Weight control:0.11), (EPINEPHRINE:Weight control:0.26), (ERYTHROPOIETIN:Weight control:0.23), (ESTROGENS, CONJUGATED:Weight control:0.24), (EVEROLIMUS:Weight control:0.14), (EVOLOCUMAB:Weight control:1.28), (GLICLAZIDE:Weight control:0.2), (HEPARIN SODIUM:Weight control:0.58), (HYDROXYZINE PAMOATE:Weight control:0.73), (HYOSCYAMINE:Weight control:0.34), (ILOPROST:Weight control:0.02), (IMIPRAMINE HYDROCHLORIDE:Weight control:0.09), (INSULIN GLULISINE:Weight control:0.27), (ISOSORBIDE:Weight control:0.57), (LERCANIDIPINE:Weight control:0.46), (LIDOCAINE HYDROCHLORIDE:Weight control:0.11), (LITHIUM CARBONATE:Weight control:0.91), (METAXALONE:Weight control:0.78), (METHYLPREDNISOLONE ACETATE:Weight control:0.06), (METHYLPREDNISOLONE SODIUM SUCCINATE:Weight control:0.45), (METRONIDAZOLE:Weight control:0.99), (MIDODRINE:Weight control:0.02), (MODAFINIL:Weight control:0.61), (MOXIFLOXACIN HYDROCHLORIDE:Weight control:0.25), (NADOLOL:Weight control:0.14), (NALOXONE HYDROCHLORIDEOXYCODONE HYDROCHLORIDE:Weight control:0.02), (NILOTINIB:Weight control:0.23), (OMEGA-3-ACID ETHYL ESTERS:Weight control:0.77), (OXCARBAZEPINE:Weight control:0.74), (OXYBUTYNIN CHLORIDE:Weight control:0.25), (PEGINTERFERON BETA-1A:Weight control:0.05), (PENICILLIN:Weight control:0.31), (POLYETHYLENE GLYCOLS:Weight control:0.71), (PRAZOSIN:Weight control:0.02), (PRIMIDONE:Weight control:0.38), (RIZATRIPTAN BENZOATE:Weight control:0.33), (ROFLUMILAST:Weight control:0.14), (SACUBITRILVALSARTAN:Weight control:0.49), (SCOPOLAMINE:Weight control:0.12), (SODIUM OXYBATE:Weight control:1.34), (TERIFLUNOMIDE:Weight control:0.69), (TOCILIZUMAB:Weight control:0.05), (UMECLIDINIUM BROMIDEVILANTEROL TRIFENATATE:Weight control:0.68), (VILAZODONE HYDROCHLORIDE:Weight control:0.41), (ZINC:Weight control:0.28), (.ALPHA.1-PROTEINASE INHIBITOR HUMAN:Weight decreased:0.11), (ACETAMINOPHENPROPOXYPHENE HYDROCHLORIDE:Weight decreased:0.22), (ALEMTUZUMAB:Weight decreased:0.04), (ALENDRONIC ACID:Weight decreased:0.15), (ALFACALCIDOL:Weight decreased:0.23), (ALISKIREN HEMIFUMARATE:Weight decreased:0.09), (AMANTADINE:Weight decreased:0.42), (AMANTADINE HYDROCHLORIDE:Weight decreased:0.16), (AMLODIPINEVALSARTAN:Weight decreased:0.16), (ANTIHISTAMINES NOS:Weight decreased:0.11), (ARIPIPRAZOLE LAUROXIL:Weight decreased:0.15), (BICALUTAMIDE:Weight decreased:1.03), (BROMAZEPAM:Weight decreased:0.54), (BROTIZOLAM:Weight decreased:0.02), (BUPRENORPHINE HYDROCHLORIDE:Weight decreased:0.88), (CALCIUM ACETATE:Weight decreased:1.06), (CALCIUM CARBONATEVITAMIN D:Weight decreased:0.31), (CALCIUM\MAGNESIUM:Weight decreased:0.12), (CALCIUM\MAGNESIUM\ZINC:Weight decreased:0.24), (CANAKINUMAB:Weight decreased:0.25), (CAPSAICIN:Weight decreased:0.1), (CAPTOPRIL:Weight decreased:0.42), (CARVEDILOL PHOSPHATE:Weight decreased:1.06), (CIMETIDINE:Weight decreased:1.06), (CLINDAMYCIN:Weight decreased:0.49), (CLOTRIMAZOLE:Weight decreased:0.72), (COD LIVER OIL:Weight decreased:0.39), (CORTICOTROPIN:Weight decreased:0.51), (CRIZOTINIB:Weight decreased:0.29), (DABIGATRAN:Weight decreased:0.22), (DACLIZUMAB:Weight decreased:0.7), (DASATINIB:Weight decreased:1.71), (DEFLAZACORT:Weight decreased:0.25), (DOXEPIN HYDROCHLORIDE:Weight decreased:0.6), (EDOXABAN TOSYLATE:Weight decreased:0.25), (EMPAGLIFLOZINMETFORMIN HYDROCHLORIDE:Weight decreased:0.18), (EPIRUBICIN:Weight decreased:0.21), (FLUDROCORTISONE ACETATE:Weight decreased:0.62), (FLUOCINONIDE:Weight decreased:0.57), (FLUTICASONE FUROATEVILANTEROL TRIFENATATE:Weight decreased:0.95), (FLUVOXAMINE MALEATE:Weight decreased:0.2), (FORMOTEROL:Weight decreased:0.01), (FORMOTEROL FUMARATE:Weight decreased:0.59), (FORMOTEROL FUMARATE\MOMETASONE FUROATE:Weight decreased:0.1), (FUSIDIC ACID:Weight decreased:0.06), (GLYCOPYRRONIUMINDACATEROL:Weight decreased:0.63), (GUANFACINE:Weight decreased:0.57), (GUANFACINE HYDROCHLORIDE:Weight decreased:0.18), (HYDROCHLOROTHIAZIDETELMISARTAN:Weight decreased:0.38), (HYDROCODONE BITARTRATEIBUPROFEN:Weight decreased:0.21), (HYDROXYCHLOROQUINE DIPHOSPHATE:Weight decreased:0.18), (HYDROXYUREA:Weight decreased:0.65), (ILOPROST:Weight decreased:0.11), (IMIPRAMINE HYDROCHLORIDE:Weight decreased:0.43), (INDAPAMIDEPERINDOPRIL:Weight decreased:0.52), (IRINOTECAN:Weight decreased:0.05), (IXAZOMIB:Weight decreased:0.14), (LANREOTIDE ACETATE:Weight decreased:0.47), (LERCANIDIPINE HYDROCHLORIDE:Weight decreased:0.63), (LEVOMEFOLATE CALCIUM:Weight decreased:0.3), (LITHIUM CARBONATE:Weight decreased:1.48), (LOMITAPIDE MESYLATE:Weight decreased:0.59), (MEDROXYPROGESTERONE:Weight decreased:0.09), (MESNA:Weight decreased:0.12), (METFORMIN HYDROCHLORIDEVILDAGLIPTIN:Weight decreased:0.13), (METHENAMINE HIPPURATE:Weight decreased:0.77), (METHYLPREDNISOLONE ACETATE:Weight decreased:0.64), (MICONAZOLE NITRATE:Weight decreased:0.05), (MORPHINE SULFATE\NALTREXONE HYDROCHLORIDE:Weight decreased:0.01), (NEBIVOLOL:Weight decreased:0.31), (NEVIRAPINE:Weight decreased:0.2), (NIRAPARIB:Weight decreased:0.36), (OSPEMIFENE:Weight decreased:0.77), (PANTOPRAZOLE MAGNESIUM:Weight decreased:1.57), (PATIROMER:Weight decreased:0.19), (PEMETREXED DISODIUM:Weight decreased:0.51), (PIROXICAM:Weight decreased:0.4), (PONATINIB:Weight decreased:0.95), (PREDNISOLONE ACETATE:Weight decreased:0.3), (PREDNISOLONEPREDNISOLONE ACETATEPREDNISOLONE SODIUM SUCCINATE:Weight decreased:0.06), (PRIMIDONE:Weight decreased:1.3), (PYRIDOXINE:Weight decreased:0.01), (QUINAPRIL:Weight decreased:0.13), (RABEPRAZOLE:Weight decreased:0.73), (RIZATRIPTAN:Weight decreased:0.13), (SACCHAROMYCES CEREVISIAE:Weight decreased:0.16), (SALMETEROL XINAFOATE:Weight decreased:0.19), (SULFAMETHOXAZOLE:Weight decreased:0.65), (TETRACYCLINE:Weight decreased:1.03), (TINZAPARIN:Weight decreased:0), (TOBRAMYCIN:Weight decreased:0.11), (TOCILIZUMAB:Weight decreased:0.23), (TROSPIUM CHLORIDE:Weight decreased:0.08), (UMECLIDINIUM BROMIDE:Weight decreased:0.02), (UMECLIDINIUM BROMIDEVILANTEROL TRIFENATATE:Weight decreased:1.04), (VALGANCICLOVIR HYDROCHLORIDE:Weight decreased:0.21), (VANCOMYCIN HYDROCHLORIDE:Weight decreased:0.09), (VINCRISTINE SULFATE:Weight decreased:0.4), (ZAFIRLUKAST:Weight decreased:0.27), (ALCOHOL:Weight increased:0.05), (AZITHROMYCIN ANHYDROUS:Weight increased:0.06), (BISOPROLOL FUMARATE:Weight increased:0.28), (BUPRENORPHINE HYDROCHLORIDENALOXONE HYDROCHLORIDE:Weight increased:0.36), (BUPROPION:Weight increased:0.18), (CALCIUM ACETATE:Weight increased:0.1), (CALCIUMVITAMIN D:Weight increased:0.1), (CEPHALEXIN:Weight increased:0.32), (CERTOLIZUMAB PEGOL:Weight increased:0.68), (CHOLESTYRAMINE:Weight increased:0.51), (CYCLOBENZAPRINE:Weight increased:0.3), (DASATINIB:Weight increased:0.14), (DEXLANSOPRAZOLE:Weight increased:1.05), (DICYCLOMINE HYDROCHLORIDE:Weight increased:0.06), (ELETRIPTAN HYDROBROMIDE:Weight increased:0.15), (ENOXAPARIN:Weight increased:0.28), (ESZOPICLONE:Weight increased:0.24), (EVOLOCUMAB:Weight increased:0.96), (FERROUS SULFATE:Weight increased:0.43), (HYDROCHLOROTHIAZIDELISINOPRIL:Weight increased:0.5), (HYDROCHLOROTHIAZIDEVALSARTAN:Weight increased:0.15), (ILOPROST:Weight increased:0.21), (LEDIPASVIRSOFOSBUVIR:Weight increased:0.22), (LINAGLIPTIN:Weight increased:0.25), (MELOXICAM:Weight increased:0.75), (METAXALONE:Weight increased:0.3), (METHYLPHENIDATE HYDROCHLORIDE:Weight increased:0.57), (METRONIDAZOLEMETRONIDAZOLE HYDROCHLORIDE:Weight increased:0.12), (MOXIFLOXACIN HYDROCHLORIDE:Weight increased:0.13), (NEBIVOLOL:Weight increased:0.17), (NILOTINIB:Weight increased:0.43), (PAROXETINE:Weight increased:0.48), (PAROXETINEPAROXETINE HYDROCHLORIDE:Weight increased:0.45), (SUMATRIPTANSUMATRIPTAN SUCCINATE:Weight increased:0.52), (TERIFLUNOMIDE:Weight increased:0.47), (THALIDOMIDE:Weight increased:0.11), (TIZANIDINE HYDROCHLORIDE:Weight increased:0.36), (UMECLIDINIUM BROMIDEVILANTEROL TRIFENATATE:Weight increased:0.2), (ABOBOTULINUMTOXINA:Wheezing:0.14), (ACENOCOUMAROL:Wheezing: 0.65), (ACETAMINOPHENPROPOXYPHENE HYDROCHLORIDE:Wheezing:0.65), (ACETAMINOPHENPROPOXYPHENE NAPSYLATE:Wheezing:0.97), (ACETAMINOPHENTRAMADOL:Wheezing:0.37), (ACITRE TIN:Wheezing:0.15), (ALBIGLUTIDE:Wheezing:1), (ALBUTEROLGUAIFENESIN:Wheezing: 0.13), (ALBUTEROLIPRATROPIUM BROMIDE:Wheezing:0.26), (AMILORIDE HYDROCHLORIDEHYDROCHLOROTHIAZIDE:Wheezing:0.29), (AMINO ACIDSDEXTROSEELECTROLYTES NOS:Wheezing:0.82), (AMLODIPINE BESYLATEHYDROCHLOROTHIAZIDEVALSARTAN:Wheezing:0.04), (ANHYDROUS CITRIC ACIDASPIRIN\SODIUM BICARBONATE:Wheezing:0.25), (ARIPIPRAZOLE LAUROXIL:Wheezing:0.07), (ASPIRIN LYSINE:Wheezing:0.39), (AVANAFIL:Wheezing:0.02), (BENZOCAINE:Wheezing:0.01), (BEN ZTROPINE MESYLATE:Wheezing:0.22), (BETAMETHASONE DIPROPIONATECALCIPOTRIENECALCIPOTRIENE HYDRATE:Wheezing:0.86), (BIFIDOBACTERIUM ANIMALIS SUBSP. LACTIS:Wheezing:0.2), (BLINATUMOMAB:Wheezing:0.79), (BRIVARACETAM:Wheezing:00.08), (BROTIZOLAM:Wheezing:0.07), (BUPRENORPHINENALOXONE:Wheezing:0.07), (BU TYLSCOPOLAMINE BROMIDE:Wheezing:1.06), (CALCIUM\MAGNESIUM\ZINC:Wheezing:0.87), (CANAGLIFL OZINMETFORMIN HYDROCHLORIDE:Wheezing:1.31), (CASPOFUNGIN:Wheezing:0.57), (CEFAZOLIN:Wheez ing:1.37), (CEFOXITINCEFOXITIN SODIUM:Wheezing:0.24), (CEFPROZIL:Wheezing:0.64), (CHLORPROMAZINE:Wheezing:0.19), (CLOMIPRAMINE HYDROCHLORIDE:Wheezing:0.37), (CURCUMIN:Wheezing:0.09), (DACLIZUMAB:Wheezi ng:1.84), (DANAZOL:Wheezing:1.03), (DEOXYRIBONUCLIEC ACID:Wheezing:0.12), (DEXMEDETOMIDINE HYDROCHLORIDE:Wheezing:0.44), (DIGITOXIN:Wheezing:0.11), (DIPYRIDAMOLE:Whee zing:0.33), (DIPYRONEPROMETHAZINE HYDROCHLORIDE:Wheezing:0.22), (DORZOLAMIDE:Wheezing:0.5), (DORZOLAMIDE HYDROCHLORIDE:Wheezing:0.18), (ECALLANTIDE:Wheezing:0), (ELTROMBOPAG OLAMINE:Wheezing:0.77), (EMPAGLIFLOZINLINAGLIPTIN:Wheezing:0.95), (EPHEDRINE HYDROCHLORIDEGUAIFENESIN:Wheezing:0.36), (EPIRUBICIN:Wheezing:0.64), (ESMO LOL HYDROCHLORIDE:Wheezing:0.27), (ESOMEPRAZOLE MAGNESIUMNAPROXEN:Wheezing:0.46), (ESTROGENS:Wheezing:0.25), (ETHINYL ESTRADIOLNORGESTREL:Wheezing:0.2), (ETORICOXIB:Wheezing:0.67), (FAMCICLOVI R:Wheezing:0.24), (FENOTEROL HYDROBROMIDEIPRATROPIUM BROMIDE:Wheezing:0.73), (FLUDROCORTISONE:Wheezing:0.32), (FLURAZEPAMFLUR AZEPAM HYDROCHLORIDE:Wheezing:0.07), (FONDAPARINUX SODIUM:Wheezing:0.42), (FUSIDIC ACID:Wheezing:1.04), (GIMERACILOTERACILTEGAFUR:Wheezing:0.11), (GLIPIZIDEM ETFORMIN HYDROCHLORIDE:Wheezing:0.28), (GLYCOPYRROLATEINDACATEROL MALEATE:Wheezing:0.51), (GLYCOPYRRONIUMINDACATEROL:Wheezing:1.61), (GUA NFACINE:Wheezing:1.16), (HUMAN C1-ESTERASE INHIBITOR:Wheezing:0.14), (HYDROCHLOROTHIAZIDESPIRONOLACTONE:Wheezing: 0.25), (HYOSCYAMINE SULFATEMETHENAMINEMETHYLENE BLUEPHENYL SALICYLATE\SODIUM PHOSPHATE, MONOBASIC, MONOHYDRATE:Wheezing:0.14), (IDELALISIB:Wheezing:0.83), (ILOPROST:Wheezing:1.68), (IMIPENEM:Wheezing:0.74), (IMIPRAMINE HYDROCHLORIDE:Wheezing:1.74), (IMIQUIMOD:Wheezing:0.27), (INDAPAMIDEPERIN DOPRIL:Wheezing:0.9), (INTERFERON:Wheezing:0.58), (INTERFERON ALFA-2B:Wheezing:0.38), (INTERFERON GAMMA-1B:Wheezing:1.5), (IODINE:Wheezing:0.23), (LAPATINIB DITOSYLATE:Wheezing:0.58), (LERCANIDIPINE:Wheezing:1.12), (LERCANIDIPINE HYDROCHLORIDE:Wheezing:0.71), (LEUCOVORIN CALCIUM:Wheezing:0.37), (LOTEPREDNOL ETABONATE:Wheezing:0.71), (LUMACAFTOR:Wheezing:0.7), (MEPOLIZUMAB:Wheezing: 1.72), (METFORMIN HYDROCHLORIDEVILDAGLIPTIN:Wheezing:0.31), (MIGLUSTAT:Wheezing:0.16), (MOE XIPRIL HYDROCHLORIDE:Wheezing:0.2), (MORPHINE SULFATE\NALTREXONE HYDROCHLORIDE:Wheezing:0.15), (NADROPARIN CALCIUM:Wheezing:0.13), (NALOXONE HYDROCHLORIDEOXYCODONE HYDROCHLORIDE:Wheezing:1.42), (NEOSTIGMINE:Wheezing:0.46), (OLMESARTAN:Wh eezing:0.18), (OSPEMIFENE:Wheezing:0.48), (OXAZEPAM:Wheezing:0.87), (OXYBUTYNIN OXYBUTYNIN CHLORIDE:Wheezing:0.12), (OXYMORPHONE:Wheezing:0.25), (PANTOPRAZOLE MAGNESIUM:Wheezing:2.61), (PAZOPANIB HYDROCHLORIDE:Wheezing:1.69), (PERINDOPRIL:Wheezing:1.17), (PIMECROLIMUS:Wh eezing:0.74), (PREDNISOLONEPREDNISOLONE ACETATEPREDNISOLONE SODIUM SUCCINATE:Wheezing:0.52), (PROTRIPTYLINE HYDROCHLORIDE:Wheezing:0.43), (PYRAZINAMIDE:Wheezing:0.77), (RADIATION THERAPY:Wheezing:0.56), (REGORAFENIB:Wheezing:0.55), (RUCAPARIB CAMSYLATE:Wheezing:0.06), (SEVELAMER HYDROCHLORIDE:Wheezing:0.13), (SODIUM CITRATE:Wheezing:0.33), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:Wheezing:0.15), (SODIUM PHOSPHATE, DIBASIC\SODIUM PHOSPHATE, MONOBASIC:Wheezing:0.73), (TASIMELTEON:Wheezing:0.13), (TAZOBACTAM:Wheezing: 0.34), (TERIFLUNOMIDE:Wheezing:1.3), (TESTOSTERONE UNDECANOATE:Wheezing:0.02), (THEOPHYLLINE:Wheezing:0.9), (THIOPENTAL SODIUM:Wheezing:0.19), (TINZAPARIN SODIUM:Wheezing:0.25), (TRANDOLAPRIL:Wheezing:0.38), (TREPROSTINIL DIOLAMINE:Wheezing:0), (TURMERIC:Wheezing:0.1), (VEMURAFENIB:Wheezing:0.18), (Y EAST:Wheezing:0.48), (ZICONOTIDE ACETATE:Wheezing:0.04), (ACETAMINOPHENCODEINE PHOSPHATE:White blood cell count decreased:0.23), (ALBUTEROL SULFATE:White blood cell count decreased:0.2), (BICALUTAMIDE:White blood cell count decreased:0.14), (BUPRENORPHINE HYDROCHLORIDENALOXONE HYDROCHLORIDE:White blood cell count decreased:0.02), (CALCIUM CARBONATECHOLECALCIFEROL:White blood cell count decreased:0.58), (CANDESARTAN:White blood cell count decreased:0.3), (CHOLESTYRAMINE:White blood cell count decreased:0), (CITALOPRAM HYDROBROMIDE:White blood cell count decreased:0.95), (CRIZOTINIB:White blood cell count decreased:0.52), (DASATINIB:White blood cell count decreased:0.57), (DEXLANSOPRAZOLE:White blood cell count decreased:0.43), (DICLOFENAC:White blood cell count decreased:0.01), (DIVALPROEX SODIUM:White blood cell count decreased:0.45), (ENALAPRIL:White blood cell count decreased:1.32), (EPINEPHRINE:White blood cell count decreased:0.27), (ESCITALOPRAM OXALATE:White blood cell count decreased:0.6), (ESZOPICLONE:White blood cell count decreased:0.32), (EVEROLIMUS:White blood cell count decreased:1.43), (EVOLOCUMAB:White blood cell count decreased:0.14), (FENOFIBRATE:White blood cell count decreased:0), (FERROUS SULFATEFERROUS SULFATE, DRIED:White blood cell count decreased:0.59), (FINGOLIMOD HYDROCHLORIDE:White blood cell count decreased:0.01), (FLUTICASONE PROPIONATE:White blood cell count decreased:0.66), (GLIPIZIDE:White blood cell count decreased:0.09), (HYDROCHLOROTHIAZIDELISINOPRIL:White blood cell count decreased:0.25), (HYDROMORPHONE:White blood cell count decreased:0.09), (IDELALISIB:White blood cell count decreased:0), (IMATINIB MESYLATE:White blood cell count decreased:0.39), (INSULIN HUMAN:White blood cell count decreased:0.33), (ITRACONAZOLE:White blood cell count decreased:0.01), (LEDIPASVIRSOFOSBUVIR:White blood cell count decreased:0.18), (LEUCOVORIN CALCIUM:White blood cell count decreased:0.92), (MEGESTROL ACETATE:White blood cell count decreased:0.09), (NILOTINIB:White blood cell count decreased:1.52), (OXYGEN:White blood cell count decreased:0.16), (POLYETHYLENE GLYCOLS:White blood cell count decreased:0.59), (REGORAFENIB:White blood cell count decreased:0.19), (SACUBITRILVALSARTAN:White blood cell count decreased:0.71), (SECUKINUMAB:White blood cell count decreased:0.45), (TIOTROPIUM BROMIDE MONOHYDRATE:White blood cell count decreased:0.17), (TOFACITINIB CITRATE:White blood cell count decreased:0.34), (TRIAMCINOLONE ACETONIDE:White blood cell count decreased:0.02), (VALGANCICLOVIR HYDROCHLORIDE:White blood cell count decreased:0.17), (ZOLPIDEMZOLPIDEM TARTRATE:White blood cell count decreased:0.28), (ACETAMINOPHENBUTALBITALCAFFEINE:Wound:0.36), (AMPHETA MINE ASPARTATEAMPHETAMINE SULFATEDEXTROAMPHETAMINE SACCHARATEDEXTROAMPHETAMINE SULFATE:Wound:0.17), (ANASTROZOLE:Wound:0.59), (ARMODAFINIL:Wound:0.15), (BIS ACODYL OR DOCUSATE SODIUM:Wound:0.86), (BORTEZOMIB:Wound:0.06), (CALCIUM CARBONATEVITAMIN D:Wound:0.11), (CANDESARTAN CILEXETILHYDROCHLOROTHIAZIDE:Wound:0.54), (CARFILZOMIB:Wound:0.26), (CHO LESTYRAMINE:Wound:0.63), (CORTISONEHYDROCORTISONE:Wound:0.26), (DASATIN 1B:Wound:0.33), (EDOXABAN TOSYLATE:Wound:0.5), (ELETRIPTAN HYDROBROMIDE:Wound:0.03), (EMPAGLIFLOZINLINAGLIPTIN:Wound:0.27), (ENOXA PARIN:Wound:1.19), (ERYTHROMYCIN:Wound:0.02), (HYDROCHLOROTHIAZIDEIRBES ARTAN:Wound:0.12), (HYDROCHLOROTHIAZIDEVALSARTAN:Wound:0.52), (HYDROX YCHLOROQUINE DIPHOSPHATE:Wound:0.16), (HYDROXYZINE PAMOATE:Wound:0.43), (HYOSCYAMINE:Wound:0.41), (IBRUTINIB:Wound:1.02), (ILOPR OST:Wound:0.35), (INDAPAMIDEPERINDOPRIL:Wound:0.18), (LERCANIDIPINE HYDROCHLORIDE:Wound:0.12), (LITHIUM CARBONATE:Wound:0.35), (LOMITAPIDE MESYLATE:Wound:0.06), (METAXALONE:Wound:0.69), (METHYLPREDNISOLONE ACETATE:Wound:0.38), (NEBIVOLOL:Wound:0.7), (NILOTINIB:Wound:0.25), (OXCARBAZ EPINE:Wound:0.56), (PATIROMER:Wound:0.2), (PENICILLIN:Wound:0.13), (PRIMIDONE:W ound:0.67), (QUETIAPINE:Wound:0.11), (RABEPRAZOLE:Wound:0.27), (ROFLUMILAST:W ound:0.2), (SACUBITRILVALSARTAN:Wound:0.35), (SODIUM OXYBATE:Wound:0.16), (SUMATRIPTAN:Wound:0.07), (TERIFLUNOMIDE:Wound:0.46), (TIOTROPIUM:Wound:0.12), (TIZANIDINE HYDROCHLORIDE:Wound:0.52), (VALGANCICLOVIR HYDROCHLORIDE:Wound:0.24), (VARENICLINE TARTRATE:Wound:0.03), (BUDESONIDE:Wound infection:0.03), (ENALAPRIL:Wound infection:0.59), (OMEPRAZOLE MAGNESIUM:Wound infection:0.02), (PYRAZINAMIDE:Wound treatment:0.58)

### <Data List 1>

The TP, TN, FP, FN, Accuracy score, Precision score, Recall score, and F_measure score are shown for each drug in the following order (Drug:TP:TN:FP:FN:Accuracy:Precision:Recall:F_measure). “#NUM!” means that “0” is included in the values and calculation cannot be therefore made.

(AZATHIOPRINE:1608:9702:0:0:1.00:1.00:1.00:1.00), (CARBOPLATIN:1576:9734:0:0:1.00:1.00:1.00:1.00), (MYCOPHENOLATE MOFETIL:1275:10035:0:0:1.00:1.00:1.00:1.00), (INFLIXIMAB:1274:10036:0:0:1.00:1.00:10.00:1.00), (LEUPROLIDE ACETATE:1074:10236:0:0:1.00:1.00:1.00:1.00), (PEMBROLIZUMAB:1062:10248:0:0:1.00:1.00:1.00:1.00), (NIVOLUMAB:939:10371:0:0:1.00:1.00:1.00:1.00), (IBANDRONATE SODIUM:806:10504:0:0:1.00:1.00:1.00:1.00), (MYCOPHENOLIC ACID:794:10516:0:0:1.00:1.00:1.00:1.00), (LEVONORGESTREL:737:10573:0:0:1.00:1.00:1.00:1.00), (TAMOXIFEN:737:10573:0:0:1.00:1.00:1.00:1.00), (USTEKINUMAB:700:10610:0:0:1.00:1.00:1.00:1.00), (HYDROCHLOROTHIAZIDEOLMESARTAN MEDOXOMIL:683:10627:0:0:1.00:1.00:1.00:1.00), (GOLIMUMAB:680:10630:0:0:1.00:1.00:1.00:1.00), (INTERFERON BETA-1A:667:10643:0:0:1.00:1.00:1.00:1.00), (DIMETHYL FUMARATE:629:10681:0:0:1.00:1.00:1.00:1.00), (NATALIZUMAB:567:10743:0:0:1.00:1.00:1.00:1.00), (MISOPROSTOL:549:10761:0:0:1.00:1.00:1.00:1.00), (BASILIXIMAB:547:10763:0:0:1.00:1.00:1.00:1.00), (ETHINYL ESTRADIOLETONOGESTREL:443:10867:0:0:1.00:1.00:1.00:1.00), (ROMIPLOSTIM:430:10880:0:0:1.00:1.00:1.00:1.00), (ELTROMBOPAG:411:10899:0:0:1.00:1.00:1.00:1.00), (A LENDRONATE SODIUMCHOLECALCIFEROL:383:10927:0:0:1.00:1.00:1.00:1.00), (DUTASTERIDETA MSULOSIN:353:10957:0:0:1.00:1.00:1.00:1.00), (ETONOGESTREL:338:10972:0:0:1.00:1.00:1.00:1.00), (ISOTRETINOIN:333:10977:0:0:1.00:1.00:1.00:1.00), (COLLAGENASE CLOSTRIDIUM HISTOLYTICUM:301:11009:0:0:1.00:1.00:1.00:1.00), (AZATHIOPRINE SODIUM:291:11019:0:0:1.00:1.00:1.00:1.00), (LIFITEGRAST:289:11021:0:0:1.00:1.00:1.00:1.00), (BOSENTANBOSENTAN MONOHYDRATE:246:11064:0:0:1.00:1.00:1.00:1.00), (EPOPROSTENOL:235:11075:0:0:1.00:1.00:1.00:1.00), (PROPYLTHIOURACIL:229:11081:0:0:1.00:1.00:1.00:1.00), (THIOTE PA:215:11095:0:0:1.00:1.00:1.00:1.00), (CHLOROQUINE:207:11103:0:0:1.00:1.00:1.00:1.00), (MIFEPRISTONE:194:11116:0:0:1.00:1.00:1.00:1.00), (DACTINOMYCIN:176:11134:0:0:1.00:1.00:1.00:1.00), (TOPOTECANTOPOTECAN HYDROCHLORIDE:170:11140:0:0:1.00:1.00:1.00:1.00), (HYDROXYPROGESTERONE CAPROATE:166:11144:0:0:1.00:1.00:1.00:1.00), (ADEFOVIR DIPIVOXIL:145:11165:0:0:1.00:1.00:1.00:1.00), (HUMAN RHO(D) IMMUNE GLOBULIN:129:11181:0:0:1.00:1.00:1.00:1.00), (DEFEROXAMINEDEFEROXAMINE MESYLATE:112:11198:0:0:1.00:1.00:1.00:1.00), (DINOPROSTONE:108:11202:0:0:1.00:1.00:1.00:1.00), (.ALPHA.-TOCOPHEROL ACETATE, DL-.BETA.-CAROTENEASCORBIC ACIDCALCIUM CARBONATECHOLECALCIFEROLCUPRIC OXIDECYANOCOBALAMINFERROUS FUMARATEFOLIC ACIDNIACINAMIDEPYRIDOXINE HYDROCHLORIDE\RIBOFLAVINTHIAMINE MONONITRATEVITAMIN A ACETATE\ZINC OXIDE:106:11204:0:0:1.00:1.00:1.00:1.00), (ASFOTASE ALFA:97:11213:0:0:1.00:1.00:1.00:1.00), (HISTRELIN ACETATE:96:11214:0:0:1.00:1.00:1.00:1.00), (VERTEPORFIN:93:11217:0:0:1.00:1.00:1.00:1.00), (TELBIVUDINE:89:11221:0:0:1.00:1.00:1.00:1.00), (OCRIPLASMIN:87:11223:0:0:1.00:1.00:1.00:1.00), (TREOSULFAN:87:11223:0:0:1.00:1.00:1.00:1.00), (ASCORBIC ACIDBIOTINMINERALSNIACINRETINOLTOCOPHEROLVITAMIN BVITAMIN D:86:11224:0:0:1.00:1.00:1.00:1.00), (BCG VACCINE:83:11227:0:0:1.00:1.00:1.00:1.00), (GALSULFASE:83:11227:0:0:1.00:1.00:1.00:1.00), (RITODRINE:81:11229:0:0:1.00:1.00:1.00:1.00), (TALC:81:11229:0:0:1.00:1.00:1.00:1.00), (MECASERMIN:74:11236:0:0:1.00:1.00:1.00:1.00), (BACILLUS CALMETTE-GUERIN SUBSTRAIN TICE LIVE ANTIGEN:56:11254:0:0:1.00:1.00:1.00:1.00), (ULIPRISTAL ACETATE:47:11263:0:0:1.00:1.00:1.00:1.00), (TIPRANAVIR:42:11268:0:0:1.00:1.00:1.00:1.00), (BLEOMYCIN HYDROCHLORIDE:40:11270:0:0:1.00:1.00:1.00:1.00), (BACILLUS CALMETTE-GUERIN ANTIGEN, UNSPECIFIED SUBSTRAIN:28:11282:0:0:1.00:1.00:1.00:1.00), (NITISINONE:21:11289:0:0:1.00:1.00:1.00:1.00), (SEMUSTINE:13:11297:0:0:1.00:1.00:1.00:1.00), (MEPHOBARBITAL:8:11302:0:0:1.00:1.00:1.00:1.00), (ISOPROPYL ALCOHOLMITOMYCIN:7:11303:0:0:1.00:1.00:1.00:1.00), (AMBROSIA ARTEMISIIFOLIA POLLEN\AMBROSIA TRIFIDA POLLEN:4:11306:0:0:1.00:1.00:1.00:1.00), (CANIS LUPUS FAMILIARIS SKIN:4:11306:0:0:1.00:1.00:1.00:1.00), (KETOROLACPHENYLEPHRINE:4:11306:0:0:1.00:1.00:1.00:1.00), (PASPALUM NOTATUM POLLEN:4:11306:0:0:1.00:1.00:1.00:1.00), (RUMEX ACETOSELLA POLLEN\RUMEX CRISPUS POLLEN:4:11306:0:0:1.00:1.00:1.00:1.00), (ADALIMUMAB:2349:8960:0:1:1.00:1.00:1.00:1.00), (TACROLIMUS:1946:9363:0:1:1.00:1.00:1.00:1.00), (CARBAMAZEPINE:1604:9705:0:1:1.00:1.00:1.00:1.00), (MESALAMINE:1413:9896:0:1:1.00:1.00:1.00:1.00), (ECULIZU MAB:925:10384:0:1:1.00:1.00:1.00:1.00), (METFORMIN HYDROCHLORIDESITAGLIPTIN PHOSPHATE:888:10421:0:1:1.00:1.00:1.00:1.00), (MYCOPHENOLATE SODIUM:731:10578:0:1:1.00:1.00:1.00:1.00), (DEFERASIROX:632:10677:0:1:1.00:1.00:1.00:1.00), (METHYLDOPA:492:10817:0:1:1.00:1.00:1.00:1.00), (AFLIBERCEPT:439:10870:0:1:1.00:1.00:1.00:1.00), (CALCIUM CHLORIDEDEXTROSEMAGNESIUM CHLORIDE\SODIUM CHLORIDE\SODIUM LACTATE:390:10919:0:1:1.00:1.00:1.00:1.00), (BECAPLERMIN:126:11183:0:1:1.00:1.00:0.99:1.00), (EQUINE THYMOCYTE IMMUNE GLOBULIN:111:11198:0:1:1.00:1.00:0.99:1.00), (PEGAPTANIB SODIUM:35:11274:0:1:1.00:1.00:0.97:0.99), (GLYCINEMANNITOLSODIUM CHLORIDE:6:11303:1:0:1.00:0.86:1.00:0.92), (ACER RUBRUM POLLEN\ACER SACCHARINUM POLLEN\ACER SACCHARUM POLLEN:4:11305:0:1:1.00:1.00:0.80:0.89), (AGROSTIS GIGANTEA POLLEN\ANTHOXANTHUM ODORATUM POLLEN\DACTYLIS GLOMERATA POLLEN\FESTUCA PRATENSIS POLLEN\LOLIUM PERENNE POLLEN\PHLEUM PRATENSE POLLEN\POA PRATENSIS POLLEN:4:11305:0:1:1.00:1.00:0.80:0.89), (AMARANTHUS RETROFLEXUS POLLEN:4:11305:0:1:1.00:1.00:0.80:0.89), (ANAS PLATYRHYNCHOS FEATHERANSER ANSER FEATHER\GALLUS GALLUS FEATHER:4:11305:0:1:1.00:1.00:0.80:0.89), (BLATELLA GERMANICA\PERIPLANETA AMERICANA:4:11305:0:1:1.00:1.00:0.80:0.89), (CARYA ILLINOINENSIS POLLEN:4:11305:0:1:1.00:1.00:0.80:0.89), (CYNODON DACTYLON POLLEN:4:11305:0:1:1.00:1.00:0.80:0.89), (DERMATOPHAGOIDES PTERONYSSINUS:4:11305:0:1:1.00:1.00:0.80:0.89), (FELIS CATUS HAIR:4:11305:0:1:1.00:1.00:0.80:0.89), (JUNIPERUS ASHEI POLLEN:4:11305:0:1:1.00:1.00:0.80:0.89), (PLANTAGO LANCEOLATA POLLEN:4:11305:0:1:1.00:1.00:0.80:0.89), (PLATANUS OCCIDENTALIS POLLEN:4:11305:0:1:1.00:1.00:0.80:0.89), (POA PRATENSIS POLLEN:4:11305:0:1:1.00:1.00:0.80:0.89), (SALIX NIGRA POLLEN:4:11305:0:1:1.00:1.00:0.80:0.89), (SORGHUM HALEPENSE POLLEN:4:11305:0:1:1.00:1.00:0.80:0.89), (ABACAVIR SUCCINATE:3:11306:0:1:1.00:1.00:0.75:0.86), (ACER NEGUNDO POLLEN:3:11306:1:0:1.00:0.75:1.00:0.86), (ACREMONIUM STRICTUM:3:11306:1:0:1.00:0.75:1.00:0.86), (ALTERNARIA ALTERNAT\A ASPERGILLUS NIGER VAR. NIGER\COCHLIOBOLUS SATIVUS\CLADOSPORIUM SPHAEROSPERMUM\PENICILLIUM CHRYSOGENUM VAR. CHRYSOGENUM:3:11306:1:0:1.00:0.75:1.00:0.86), (AUREOBASIDIUM PULLULANS VAR. PULLUTANS\COCHLIOBOLUS SPICIFER\GIBBERELLA FUJIKUROI\MUCOR PLUMBEUS\RHIZOPUS STOLONIFER:3:11306:1:0:1.00:0.75:1.00:0.86), (CHENOPODIUM ALBUM POLLEN:3:11306:1:0:1.00:0.75:1.00:0.86), (PERIPLANETA AMERICANA:3:11306:1:0:1.00:0.75:1.00:0.86), (QUERCUS RUBRA POLLEN:3:11306:1:0:1.00:0.75:1.00:0.86), (STEMPHYLIUM SOLANI:3:11306:1:0:1.00:0.75:1.00:0.86), (ULMUS AMERICANA POLLEN:3:11306:1:0:1.00:0.75:1.00:0.86), (TECHNETIUM TC-99M PYROPHOSPHATE:1:11308:0:1:1.00:1.00:0.50:0.67), (ETANERCEPT:1521:9787:0:2:1.00:1.00:1.00:1.00), (PAROXETINE HYDROCHLORIDE:1505:9803:0:2:1.00:1.00:1.00:1.00), (VALPROATE SODIUM:1062:10246:0:2:1.00:1.00:1.00:1.00), (SIROLIMUS:857:10451:0:2:1.00:1.00:1.00:1.00), (SOMATROPIN:846:10462:0:2:1.00:1.00:1.00:1.00), (ALBUMIN HUMANPHENOL:4:11304:0:2:1.00:1.00:0.67:0.80), (EPICOCCUM NIGRUM:4:11304:0:2:1.00:1.00:0.67:0.80), (DELAVIRDINE MESYLATE:3:11305:0:2:1.00:1.00:0.60:0.75), (JUNIPERUS VIRGINIANA POLLEN:3:11305:1:1:1.00:0.75:0.75:0.75), (PINUS STROBUS POLLEN:3:11305:1:1:1.00:0.75:0.75:0.75), (POPULUS DELTOIDES POLLEN:3:11305:0:2:1.00:1.00:0.60:0.75), (AMARANTHUS RETROFLEXUS POLLEN\CHENOPODIUM ALBUM POLLEN\XANTHIUM STRUMARIUM POLLEN:2:11306:2:0:1.00:0.50:1.00:0.67), (BETULA NIGRA POLLEN\CARYA OVATA POLLEN\FAGUS GRANDIFOLIA\FRAXINUS AMERICANA POLLEN\POPULUS DELTOIDES POLLEN\QUERCUS RUBRA POLLEN\ULMUS AMERICANA POLLEN:2:11306:2:0:1.00:0.50:1.00:0.67), (CARYA OVATA POLLEN:2:11306:2:0:1.00:0.50:1.00:0.67), (FELIS CATUS DANDER:2:11306:0:2:1.00:1.00:0.50:0.67), (6-ACETYLMORPHINE:1:11307:1:1:1.00:0.50:0.50:0.50), (AVOBENZONEDIHYDROXYAC ETONEDIMETHICONE COPOLYOLHEXYL LAURATEHOMOSALATEOCTISALATEOCTOCRYLENEOXYBENZONEPROPYLENE GLYCOL:1:11307:1:1:1.00:0.50:0.50:0.50), (COTININE:1:11307:1:1:1.00:0.50:0.50:0.50), (TERIPARATIDE:881:10426:0:3:1.00:1.00:1.00:1.00), (RANIBIZUMAB:556:10751:0:3:1.00:1.00:0.99:1.00), (FIBRINOGEN HUMANTHROMBIN HUMAN:158:11149:0:3:1.00:1.00:0.98:0.99), (CHONDROITIN SULFATE A\HYALURONATE SODIUM:10:11297:0:3:1.00:1.00:0.77:0.87), (CANIS LUPUS FAMILIARIS DANDER\CANIS LUPUS FAMILIARIS HAIR:4:11303:0:3:1.00:1.00:0.57:0.73), (DERMATOPHAGOIDES FARINAE:4:11303:0:3:1.00:1.00:0.57:0.73), (ETHYLMORPHINE:2:11305:2:1:1.00:0.50:0.67:0.57), (3-METHOXYPHENCYCLIDINE:1:11306:1:2:1.00:0.50:0.33:0.40), (COCAINE HYDROCHLORIDE:1:11306:2:1:1.00:0.33:0.50:0.40), (EMTRICITABINETENOFOVIRZI DOVUDINE:1:11306:3:0:1.00:0.25:1.00:0.40), (GADOFOSVESET TRISODIUM:1:11306:1:2:1.00:0.50:0.33:0.40), (MAGNESIUM ACETATE TETRAHYDRATE\POTASSIUM ACETATE\SODIUM CHLORIDE:1:11306:3:0:1.00:0.25:1.00:0.40), (PENTACHLOROPHENOL:1:11306:3:0:1.00:0.25:1.00:0.40), (SALSOLA KALI POLLEN:1:11306:1:2:1.00:0.50:0.33:0.40), (GLYCOLIC ACIDSALICYLIC ACID:0:11307:1:2:1.00:0.00:0.00:#NUM!), (METHOTREXATE:2695:8611:0:4:1.00:1.00:1.00:1.00), (RIVAROXABAN:2372:8934:0:4:1.00:1.00:1.00:1.00), (ALTERNARIA ALTERNATA:4:11302:0:4:1.00:1.00:0.50:0.67), (JUGLANS NIGRA POLLEN:4:11302:0:4:1.00:1.00:0.50:0.67), (ARTEMISIA VULGARIS POLLEN:3:11303:1:3:1.00:0.75:0.50:0.60), (AVOBENZONEHOMOSALATEOCTINOXA TEOCTISALATEOCTOCRYLENEOXYBENZONE:2:11304:1:3:1.00:0.67:0.40:0.50), (FI LANESIB:2:11304:2:2:1.00:0.50:0.50:0.50), (FOSAMPRENAVIRRITONAVIR:2:11304:1:3:1.00:0.67:0.40:0.50), (LAMIVUDINENEVIRAPINEZIDOVUDINE:2:11304:2:2:1.00:0.50:0.50:0.50), (BENZOYLECGONINE:1:11305:1:3:1.00:0.50:0.25:0.33), (BISMUTH SUBCITRATE:1:11305:3:1:1.00:0.25:0.50:0.33), (BISMUTH TRISALICYLATE:1:11305:1:3:1.00:0.50:0.25:0.33), (CHROMIC CHLORIDECUPRIC CHLORIDEMANGANESE CHLORIDE\ZINC CHLORIDE:1:11305:3:1:1.00:0.25:0.50:0.33), (IOTHALAMIC ACID:1:11305:3:1:1.00:0.25:0.50:0.33), (PARALDEHYDE:1:11305:3:1:1.00:0.25:0.50:0.33), (RABIES IMMUNE GLOBULIN (HUMAN):1:11305:1:3:1.00:0.50:0.25:0.33), (EFAVIRENZLAMIVUDINEZIDOVUDINE: 0:11306:3:1:1.00:0.00:0.00:#NUM!), (ALENDRONATE SODIUM:1828:9477:0:5:1.00:1.00:1.00:1.00), (DENOSUMAB:1817:9488:0:5:1.001.00:1.00:1.00), (APIXABAN:1652:9653:0:5:1.00:1.00:1.00:1.00), (RIPASUDIL HYDROCHLORIDE DIHYDRATE:6:11299:2:3:1.00:0.75:0.67:0.71), (AMLODIPINELOSARTAN POTASSIUM:3:11302:2:3:1.00:0.60:0.50:0.55), (ABACAVIRLAMIVUDINEZIDOVUDIN E:2:11303:5:0:1.00:0.29:1.00:0.44), (ACETAMINOPHEN\ASPIRINDIPHENHYDRAMINE CITRATE:2:11303:2:3:1.00:0.50:0.40:0.44), (ASCORBIC ACIDFERROUS FUMARATEFOLIC ACID:2:11303:5:0:1.00:0.29:1.00:0.44), (HUMAN PAPILLOMAVIRUS TYPE 11 L1 CAPSID PROTEIN ANTIGENHUMAN PAPILLOMAVIRUS TYPE 16 L1 CAPSID PROTEIN ANTIGENHUMAN PAPILLOMAVIRUS TYPE 18 L1 CAPSID PROTEIN ANTIGENHUMAN PAPILLOMAVIRUS TYPE 31 L1 CAPSID PROTEIN ANTIGENHUMAN PAPILLOMAVIRUS TYPE 33 L1 CAPSID PROTEIN ANTIGENHUMAN PAPILLOMAVIRUS TYPE:2:11303:1:4:1.00:0.67:0.33:0.44), (LAMIVUDINENEVIRAPINESTAVUDINE:2:11303:2:3:1.00:0.50:0.40:0.44), (OXPRENOLOL:2:11303:2:3:1.00:0.50:0.40:0.44), (SIMVAST ATINVALSARTAN:2:11303:2:3:1.00:0.50:0.40:0.44), (ACETAMINOPHENCODEINEPH ENYLEPHRINE:1:11304:1:4:1.00:0.50:0.20:0.29), (AUREOBASIDIUM PULLULANS VAR. PULLUTANS:1:11304:1:4:1.00:0.50:0.20:0.29), (AVOBENZONEHOMOSALATEOCTISA LATEOXYBENZONE:1:11304:1:4:1.00:0.50:0.20:0.29), (BUPRANOLOL:1:11304:3:2:1.00:0.25:0.33:0.29), (CARBOPROST:1:11304:1:4:1.00:0.50:0.20:0.29), (DABRAFENIBTRA METINIB:1:11304:3:2:1.00:0.25:0.33:0.29), (DASABUVIROMBITASVIRPARITAPREVI RRIBAVIRINRITONAVIR:1:11304:3:2:1.00:0.25:0.33:0.29), (DEXPANTHENOLNIACIN AMIDEPYRIDOXINE HYDROCHLORIDE\RIBOFLAVINTHIAMINE HYDROCHLORIDE:1:11304:3:2:1.00:0.25:0.33:0.29), (ELECTROLYTES NOSMINERALSSORBITOL:1:11304:3:2:1.00:0.25:0.33:0.29), (ETHYLPHENIDATE:1:11304:3:2:1.00:0.25:0.33:0.29), (OLSALAZINE:1:11304:3:2:1.00:0.25:0.33:0.29), (TECHNE TIUM TC-99M EXAMETAZIME:1:11304:2:3:1.00:0.33:0.25:0.29), (UROFOLLITROPIN:1:11304:1:4:1.00:0.50:0.20:0.29), (VON WILLEBRAND FACTOR (RECOMBINANT):1:11304:1:4:1.00:0.50:0.20:0.29), (ALPHA-TOCOPHEROL ACETATEASCORBIC ACIDBIOTINCHOLECALCIFEROLCYANOCOBALAMINDEXPANTHENOLFOLIC ACIDNIACINAMIDEPHYTONADIONEPYRIDOXINE HYDROCHLORIDE\RIBOFLAVIN 5′-PHOSPHATE SODIUMTHIAMINE HYDROCHLORIDEVITAMIN A PALMITATE:0:11305:2:3:1.00:0.00:0.00:#NUM!), (ARTICAINE HYDROCHLORIDEEPINEPHRINE HYDROCHLORIDE:0:11305:2:3:1.00:0.00:0.00:#NUM!), (DACETUZUMAB:0:11305:4:1:1.00:0.00:0.00:#NUM!), (HOMEOPATHICSZINC ACETATE ANHYDROUSZINC GLUCONATE:0:11305:3:2:1.00:0.00:0.00:#NUM!), (TANESPIMYCIN:0:11305:4:1:1.00:0.00:0.00:#NUM!), (SITAGLIPTIN PHOSPHATE:1518:9786:0:6:1.00:1.00:1.00:1.00), (IMATINIB:349:10955:0:6:1.00:1.00:0.98:0.99), (OXYTOCIN:318:10986:0:6:1.00:1.00:0.98:0.99), (INDINAVIR SULFATE:39:11265:0:6:1.00:1.00:0.87:0.93), (TRELAGLIPTIN SUCCINATE:5:11299:2:4:1.00:0.71:0.56:0.63), (ATAZANAVIR SULFATERITONAVIR:4:11300:2:4:1.00:0.67:0.50:0.57), (POLYURETHANE:4:11300:1:5:1.00:0.80:0.44:0.57), (SOFOSBUVIRVELPATASVIRVOXILAPREVIR:4:11300:1:5:1.00:00.80:0.44:0.57), (BIMEKIZUMAB:3:11301:0:6:1.00:1.00:0.33:0.50), (CETALKONIUM CHLORIDECHOLINE SALICYLATEMENTHOL:3:11301:1:5:1.00:0.75:0.38:0.50), (AGALSIDASE ALFA:2:11302:2:4:1.00:0.50:0.33:0.40), (ALUMINUM ZIRCONIUM TETRACHLOROHYDREX GLY:2:11302:1:5:1.00:0.67:0.29:0.40), (AMITRIPTYLINE HYDROCHLORIDECHLORDIAZEPOXIDE HYDROCHLORIDE:2:11302:2:4:1.00:0.50:0.33:0.40), (CABOTEGRAVIR:2:11302:3:3:1.00:0.40:0.40:0.40), (CHLORAMPHENICOL PALMITATE:2:11302:3:3:1.00:0.40:0.40:0.40), (HEPATITIS A VIRUS STRAIN CR 326F ANTIGEN (FORMALDEHYDE INACTIVATED):2:11302:2:4:1.00:0.50:0.33:0.40), (IODIZED OIL:2:11302:0:6:1.00:1.00:0.25:0.40), (.ALPHA.-TOCOPHEROL ACETATEASCORBIC ACIDBIOTINCHOLECALCIFEROLCYANOCOBALAMINDEXPANTHENOLFOLIC ACIDNIACINAMIDEPHYTONADIONEPYRIDOXINE HYDROCHLORIDE\RIBOFLAVIN 5′-PHOSPHATE SODIUMTHIAMINE HYDROCHLORIDEVITAMIN A PALMITATE:1:11303:0:6:1.00:1.00:0.14:0.25), (BICARBONATE IONCALCIUM CATIONLACTIC ACIDMAGNESIUM CATION\SODIUM CHLORIDE:1:11303:1:5:1.00:0.50:0.17:0.25), (CHLORODEHYDROMETHYLTESTOSTER ONE:1:11303:3:3:1.00:0.25:0.25:0.25), (CLOPIDOGREL HYDROBROMIDE:1:11303:3:3:1.00:0.25:0.25:0.25), (ETHOTOIN:1:11303:2:4:1.00:0.33:00.20:0.25), (HYPOCHLORITE ION:1:11303:3:3:1.00:0.25:0.25:0.25), (PHOSPHAZIDE:1:11303:2:4:1.00:0.33:0.20:0.25), (POLYETHYLENE GLYCOL 400POLYVINYL ALCOHOL:1:11303:2:4:1.00:0.33:0.20:0.25), (TECHNETIUM TC-99M TILMANOCEPT:1:11303:3:3:1.00:0.25:0.25:0.25), (TOSITUMOMABTOSITUMOMAB I-131:1:11303:3:3:1.00:0.25:0.25:0.25), (ZINC ACETATE ANHYDROUSZINC GLUCONATE:1:11303:3:3:1.00:0.25:0.25:0.25), (10-HYDROXYCAMPTOTHECIN:0:11304:4:2:1.00:0.00:0.00:#NUM!), (AUTOLOGOUS CULTURED CHONDROCYTESSUS SCROFA COLLAGEN:0:11304:4:2:1.00:0.00:0.00:#NUM!), (LESTAURTINIB:0:11304:5:1:1.00:0.00:0.00:#NUM!), (HUMAN IMMUNOGLOBULIN G:1811:9492:0:7:1.00:1.00:1.00:1.00), (FINASTERIDE:1494:9809:0:7:1.00:1.00:1.00:1.00), (EMPAGLIFLOZIN:494:10809:0:7:1.00:1.00:0.99:0.99), (INSULIN GLARGINELIXISENATIDE:8:11295:2:5:1.00:0.80:0.62:0.70), (AVOBENZONEOCTINO XATEOCTISALATE:3:11300:0:7:1.00:1.00:0.30:0.46), (EFMOROCTOCOG ALFA:3:11300:1:6:1.00:0.75:0.33:0.46), (IOBENGUANE I-131:3:11300:1:6:1.00:0.75:0.33:0.46), (MITRAGYNINEHERBALS:3:11300:1:6:1.00:0.75:00.33:0.46), (OCTINOXATE\ZINC OXIDE:3:11300:0:7:1.00:1.00:0.30:0.46), (PEGINESATIDE:3:11300:0:7:1.00:1.00:0.30:0.46), (ALLANTOINOCTENIDINE:2:11301:2:5:1.00:0.50:0.29:0.36), (BUPIVACAINE HYDROCHLORIDEFENTANYL CITRATE:2:11301:2:5:1.00:0.50:0.29:0.36), (HUMAN RABIES VIRUS IMMUNE GLOBULIN:2:11301:0:7:1.00:1.00:0.22:0.36), (LUTROPIN ALFA:2:11301:0:7:1.00:1.00:0.22:0.36), (NIACINAMIDEPYRIDOXINE HYDROCHLORIDE\RIBOFLAVINTHIAMINE HYDROCHLORIDE:2:11301:3:4:1.00:0.40:0.33:0.36), (OCTINOXATEOCTOCRYLENE:2:11301:1:6:1.00:0.67:0.25:0.36), (SODIUM SULFIDE:2:11301:2:5:1.00:0.50:0.29:0.36), (ACETAMINOPHENDEXTROMETHORPHA NDOXYLAMINEPSEUDOEPHEDRINE:1:11302:3:4:1.00:0.25:0.20:0.22), (ALANINEAR GININEASPARTIC ACIDCYSTEINE HYDROCHLORIDEGLUTAMIC ACIDGLYCINEHISTIDINEISOLEUCINELEUCINELYSINE ACETATEMETHIONINEN-ACETYL-TYROSINEPHENYLALANINEPROLINESERINETAURINETHREONINETRYPTOPHA NTYROSINEVALINE:1:11302:3:4:1.00:0.25:0.20:0.22), (ALISPORIVIR:1:11302:5:2:1.00:0.17:0.33:0.22), (AVOBENZONEHOMOSALATEOCTINOXATEOCTISALATEOXYBE NZONE:1:11302:2:5:1.00:0.33:0.17:0.22), (EMACTUZUMAB:1:11302:3:4:1.00:0.25:0.20:0.22), (GLYCERINHYPROMELLOSESPOLYETHYLENE GLYCOL 400TETRAHYDROZOLINE HYDROCHLORIDE:1:11302:1:6:1.00:0.50:0.14:0.22), (IODINE POVACRYLEXISOPROPYL ALCOHOL:1:11302:2:5:1.00:0.33:0.17:0.22), (METHANDROSTENOLONE:1:11302:2:5:1.00:0.33:0.17:0.22), (OCTINOXATEOCTISALATE\ZINC OXIDE:1:11302:2:5:1.00:0.33:0.17:0.22), (RUBIDIUM CHLORIDE RB-82:1:11302:2:5:1.00:0.33:0.17:0.22), (SODIUM IODIDE I-123:1:11302:0:7:1.00:1.00:0.13:0.22), (TRISODIUM CITRATE DIHYDRATE:1:11302:2:5:1.00:0.33:0.17:0.22), (ALCOHOLCHLORHEXIDINE GLUCONATE:0:11303:1:6:1.00:0.00:0.00:#NUM!), (CILENGITIDE:0:11303:4:3:1.00:0.00:0.00:#NUM!), (CURCUMINDIETARY SUPPLEMENT:0:11303:4:3:1.00:0.00:0.00:#NUM!), (VELPATASVIR:0:11303:4:3:1.00:0.00:0.00:#NUM!), (VITAMIN A ACETATE:0:11303:2:5:1.00:0.00:0.00:#NUM!), (ONDANSETRON:2482:8820:0:8:1.00:1.00:1.00:1.00), (DIENOGESTETHINYL ESTRADIOL:63:11239:0:8:1.00:1.00:0.89:0.94), (ALEFACEPT:5:11297:3:5:1.00:0.63:0.50:0.56), (.ALPHA.-TOCOPHEROL SUCCINATE, D-ASCORBIC ACIDBIOTINCALCIUM PANTOTHENATECOBALAMINFOLIC ACIDNIACINAMIDEPYRIDOXINE HYDROCHLORIDE\RIBOFLAVINSELENOCYSTEINETHIAMINE MONONITRATE\ZINC CITRATE:4:11298:2:6:1.00:0.67:0.40:0.50), (OCTINOXATEOXYBENZONE:4:11298:2:6:10.00:0.67:0.40:0.50), (SARILUMAB:4:11298:2:6:1.00:0.67:0.40:0.50), (ALUMINUM ZIRCONIUM TRICHLOROHYDREX GLY:3:11299:0:8:1.00:1.00:0.27:0.43), (DELAVIRDINE:3:11299:1:7:1.00:0.75:0.30:0.43), (SECOBARBITAL SODIUM:3:11299:3:5:1.00:0.50:0.38:0.43), (TYPHOID VACCINE:3:11299:2:6:1.00:0.60:0.33:0.43), (CAFFEINEINDOMETHACINPROCHLORP ERAZINE MALEATE:2:11300:3:5:1.00:0.40:0.29:0.33), (CALFACTANT:2:11300:1:7:1.00:0.67:0.22:0.33), (CEFTOBIPROLE:2:11300:2:6:1.00:0.50:0.25:0.33), (CLOPREDNOL:2:11300:6:2:1.00:0.25:0.50:0.33), (EUGENOL:2:11300:1:7:1.00:0.67:0.22:0.33), (GANIRELIX:2:11300:0:8:1.00:1.00:0.20:0.33), (HOMOSALATEOCTINOXATEOCTISALATEOXYBENZONE:2:11300:1:7:1.00:0.67:0.22:0.33), (INDAPAMIDE HEMIHYDRATE:2:11300:2:6:1.00:0.50:0.25:0.33), (KETOBEMIDONE:2:11300:3:5:1.00:0.40:0.29:0.33), (PANCRELIPASETYROSINE:2:11300:5:3:1.00:0.29:0.40:0.33), (PROMET HAZINE TEOCLATE:2:11300:1:7:1.00:0.67:0.22:0.33), (ACETIC ACIDCALCIUM CHLORIDEDEXTROSEMAGNESIUM CHLORIDE\POTASSIUM CHLORIDE\SODIUM CHLORIDE:1:11301:3:5:1.00:0.25:0.17:0.20), (ALANINEARGININEGLYCINEHISTIDI NEISOLEUCINELEUCINELYSINE HYDROCHLORIDEMETHIONINEPHENYLALANINEPROLINESERINETHREONINE TRYPTOPHANTYROSINEVALINE:1:11301:3:5:1.00:0.25:0.17:0.20), (ANHYDROUS CITRIC ACIDBICARBONATE IONMAGNESIUM CATION:1:11301:3:5:1.00:0.25:0.17:0.20), (DILOXANIDE FUROATETINIDAZOLE:1:11301:3:5:1.00:0.25:0.17:0.20), (GANITUMAB:1:11301:3:5:1.00:0.25:0.17:0.20), (KETANSERIN:1:11301:2:6:1.00:0.33:0.14:0.20), (RIGOSERTIB:1:11301:3:5:1.00:0.25:0.17:0.20), (TRENBOLONE:1:11301:2:6:1.00:0.33:0.14:0.20), (ALLERGE NIC EXTRACT-CANDIDA:0:11302:3:5:1.00:0.00:0.00:#NUM!), (FARLETUZUMAB:0:11302:4:4:1.00:0.00:0.00:#NUM!), (EPROSARTAN MESYLATEHYDROCHLOROTHIAZIDE:4:11297:4:5:1.00:0.50:0.44:0.47), (MOXIFLOX ACINTRIAMCINOLONE:4:11297:6:3:1.00:0.40:0.57:0.47), (NIACINAMIDERIBOFLAVI NTHIAMINE HYDROCHLORIDE:4:11297:0:9:1.00:1.00:0.31:0.47), (PIPOTIAZINE:4:11297:5:4:1.00:0.44:0.50:0.47), (AMOBARBITAL:3:11298:2:7:1.00:0.60:0.30:0.40), (AMPHETAMINE ASPARTATE MONOHYDRATEAMPHETAMINE SULFATEDEXTROAMPHETAMINE SACCHARATEDEXTROAMPHETAMINE SULFATE:3:11298:8:1:1.00:0.27:0.75:0.40), (ATLANTIC SALMON OIL:3:11298:2:7:1.00:0.60:0.30:0.40), (SIPONIMOD:3:11298:1:8:1.00:0.75:0.27:0.40), (SI RUKUMAB:3:11298:3:6:1.00:0.50:0.33:0.40), (ACETAMINOPHENDIPHENHYDRAMINE HYDROCHLORIDEPSEUDOEPHEDRINE HYDROCHLORIDE:2:11299:1:8:1.00:0.67:0.20:0.31), (ASPIRINDIPHENHYDRAMINE:2:11299:1:8:1.00:0.67:0.20:0.31), (CARBON MONOXIDE:2:11299:0:9:1.00:1.00:0.18:0.31), (DROPERIDOLFENTANYL CITRATE:2:11299:1:8:1.00:0.67:0.20:0.31), (DYCLONINE HYDROCHLORIDE:2:11299:7:2:1.00:0.22:0.50:0.31), (HUMAN ROTAVIRUS A TYPE G1P7(5) STRAIN W179 LIVE ANTIGENHUMAN ROTAVIRUS A TYPE G2P7(5) STRAIN SC2 LIVE ANTIGENHUMAN ROTAVIRUS A TYPE G3P7(5) STRAIN W178 LIVE ANTIGENHUMAN ROTAVIRUS A TYPE G4P7(5) STRAIN BRB LIVE ANTIGENHUMAN ROTAVIRUS A TYPE G6P1A(8) STRAIN W179 LIVE ANTIGEN:2:11299:1:8:1.00:0.67:0.20:0.31), (MANGANESE GLUCONATE:2:11299:2:7:1.00:0.50:0.22:0.31), (NORETHANDROLONE:2:11299:2:7:1.00:0.50:0.22:0.31), (PEGINTERFERON LAMBDA-1A:2:11299:6:3:1.00:0.25:0.40:0.31), (ARSENIC:1:11300:3:6:1.00:0.25:0.14:0.18), (AUTO LOGOUS CULTURED CHONDROCYTES:1:11300:2:7:1.00:0.33:0.13:0.18), (AVOBENZONEOCTOCRYLENEO XYBENZONE:1:11300:1:8:1.00:0.50:0.11:0.18), (CALCIUM CLDEXTROSEMAGNESIUM CLSODIUM BICARSODIUM CLSODIUM LACT:1:11300:4:5:1.00:0.20:0.17:0.18), (CANDIDA ALBICANS:1:11300:3:6:1.00:0.25:0.14:0.18), (CETRORELIX:1:11300:1:8:1.00:0.50:0.11:0.18), (CHENODIOL:1:11300:3:6:1.00:0.25:0.14:0.18), (CHLORPHENIRAMINE MALEATEHYDROCODONE BITARTRATE:1:11300:1:8:1.00:0.50:0.11:0.18), (CUPRIC CHLORIDEMANGANESE CHLORIDE\POTASSIUM IODIDE\SODIUM FLUORIDE\SODIUM SELENITE\ZINC CHLORIDE:1:11300:1:8:1.00:0.50:0.11:0.18), (DALOTUZUMAB:1:11300:3:6:1.00:0.25:0.14:0.18), (ERGOCALCIFEROLPHYTONADIONETOCOPHEROLVITAMIN A PALMITATE:1:11300:1:8:1.00:0.50:0.11:0.18), (FLUOCORTOLONE:1:11300:3:6:1.00:0.25:0.14:0.18), (HOMEOPATHICSZINC ACETATE\ZINC GLUCONATE:1:11300:3:6:1.00:0.25:0.14:0.18), (LAMIVUDINETENOFOVIR:1:11300:0:9:1.00:1.00:0.10:0.18), (LIDOCAINEPHENYLEPHRINE:1:11300:1:8:1.00:0.50:0.11:0.18), (MIFAMURTIDE:1:11300:6:3:1.00:0.14:0.25:0.18), (MOLYBDENUM MO-99\TECHNETIUM TC-99M:1:11300:1:8:1.00:0.50:0.11:0.18), (PRACINOSTAT:1:11300:3:6:1.00:0.25:0.14:0.18), (HEXAMINOLEVULINATE HYDROCHLORIDE:0:11301:2:7:1.00:0.00:0.00:#NUM!), (NELFINAVIR MESYLATE:66:11234:0:10:1.00:1.00:0.87:0.93), (RITODRINE HYDROCHLORIDE:39:11261:1:9:1.00:0.98:0.81:0.89), (MENOTROPINS:15:11285:3:7:1.00:0.83:0.68:0.75), (PIPOTIAZINE PALMITATE:4:11296:6:4:1.00:0.40:0.50:0.44), (TRAMAZOLINE HYDROCHLORIDE:4:11296:0:10:1.00:1.00:0.29:0.44), (FLUSPIRILENE:3:11297:1:9:1.00:0.75:0.25:0.38), (METHOHEXITAL SODIUM:3:11297:1:9:1.00:0.75:0.25:0.38), (OXYCODONE TEREPHTHALATE:3:11297:2:8:1.00:0.60:0.27:0.38), (ACEPROMAZINE:2:11298:3:7:1.00: 0.40:0.22:0.29), (AVOBENZONEOCTINOXATEOCTISALATEOXYBENZONE:2:11298: 1:9:1.00:0.67:0.18:0.29), (FLUTEMETAMOL F-18:2:11298:1:9:1.00:0.67:0.18:0.29), (OPICAPONE:2:11298:10:0:1.00:0.17:1.00:0.29), (PE GADEMASE BOVINE:2:11298:1:9:1.00:0.67:0.18:0.29), (SERMORELIN ACETATE:2:11298:4:6:1.00:0.33:0.25:0.29), (ACETAMINOPHEN\CAFFEINEPHENYLEP HRINE HYDROCHLORIDE:1:11299:2:8:1.00:0.33:0.11:0.17), (ACETAMINOPHENCHLORPHENI RAMINE MALEATEDEXTROMETHORPHAN HYDROBROMIDEDOXYLAMINE SUCCINATEPHENYLEPHRINE HYDROCHLORIDE:1:11299:3:7:1.00:0.25:0.13:0.17), (ALVOCIDIB:1:11299:3:7:1.00:0.25: 0.13:0.17), (AVOBENZONEECAMSULEOCTOCRYLENE:1:11299:3:7:1.00:0.25:0.13:0.17), (CHLORHEXIDINE GLUCONATECHLOROCRESOLHEXAMIDINE DIISETHIONATE:1:11299:2:8:1.00:0.33:0.11:0.17), (CLIOQUINOL:1:11299:3:7:1.00:0.25: 0.13:0.17), (DEXTRAN 70POLYETHYLENE GLYCOL 400POVIDONETETRAHYDROZOLINE HYDROCHLORIDE:1:11299:1:9:1.00:0.50:0.10:0.17), (ENBUCRILATE:1:11299:3:7:1.00:00.25:0.13:0.17), (ENTOSPLETINIB:1:11299:3:7:1.00:0.25:0.13:0.17), (ETHAMBUTOL HYDROCHLORIDEISONIAZIDPYRAZINAMIDERIFAMPIN:1:11299:3:7:1.00:0.25:0.13: 0.17), (FURAZOLIDONE:1:11299:3:7:1.00:0.25:0.13:0.17), (LAMIVUDINETENOFOVIR DISOPROXIL FUMARATE:1:11299:2:8:1.00:0.33:0.11:0.17), (MAGNESIUM CHLORIDE\POTASSIUM CHLORIDE\POTASSIUM PHOSPHATE\SODIUM ACETATE\SODIUM CHLORIDE\SODIUM GLUCONATE\SODIUM PHOSPHATE, DIBASIC:1:11299:3:7:1.00:0.25:0.13:0.17), (MEASLES VIRUS STRAIN ENDERS' ATTENUATED EDMONSTON LIVE ANTIGENMUMPS VIRUS STRAIN B LEVEL JERYL LYNN LIVE ANTIGENRUBELLA VIRUS STRAIN WISTAR RA 27/3 LIVE ANTIGENVARICELLA-ZOSTER VIRUS STRAIN OKA/MERCK LIVE ANTIGEN:1:11299:1:9:1.00:0.50:0.10:0.17), (MITRAGYNINE:1:11299:3:7:1.00:0.25:0.13: 0.17), (RACEPINEPHRINE HYDROCHLORIDE:1:11299:4:6:1.00:0.20:0.14:0.17), (SIMEPREVIR SODIUM:1:11299:6:4:1.00:0.14:0.20:0.17), (SODIUM BENZOATE\SODIUM PHENYLACETATE:1:11299:3:7:1.00:0.25:0.13:0.17), (TECHNETIUM TC-99M MEBROFENIN:1:11299:1:9:1.00:0.50:0.10:0.17), (ASPERGILLUS FUMIGATUS:0:11300:1:9:1.00:0.00:0.00:#NUM!), (ERGONOVINEOXYTOCIN:0:11300:4: 6:1.00:0.00:0.00:#NUM!), (FOSBRETABULIN TROMETHAMINE:0:11300:8:2:1.00:0.00:0.00:#NUM!), (LEVODOPAUNSPECIFIED INGREDIENT:0:11300:4:6:1.00:0.00:0.00:#NUM!), (MITOGUAZONE:0:11300:4:6:1.00:0.0 0:0.00:#NUM!), (OXYMETAZOLINE HYDROCHLORIDETETRACAINE HYDROCHLORIDE:0:11300:3:7:1.00:0.00:0.00:#NUM!), (ENTECAVIR:553:10746:0:11:1.00:1.00:0.98:0.99), (ADEFOVIR:31:11268:2:9:1.00:0.94:0.78:0.85), (HALOTHANE:8:11291: 2:9:1.00:0.80:0.47:0.59), (INDIGOTINDISULFONATE SODIUM:8:11291:4:7:1.00:0.67:0.53:0.59), (METFORMINVILDAGLIPTIN:5:11294:3:8:1.00:0.63:0.38:0.48), (AMBROSIA ARTEMISIIFOLIA POLLEN:4:11295:0:11:1.00:1.00:0.27:0.42), (HYDROCHLOROTHIAZIDEZOFENOPRIL CALCIUM:4:11295:1:10:1.00:0.80:0.29:0.42), (CHLORPHENIRAMINE HYDROCHLORIDEHYDROCODONE:3:11296:1:10:1.00:0.75:0.23:0.35), (GRAPEFRUIT: 3:11296:2:9:1.00:0.60:0.25:0.35), (ISATUXIMAB:3:11296:1:10:1.00:0.75:0.23:0.35), (MEP HENOXALONE:3:11296:1:10:1.00:0.75:0.23:0.35), (ORTERONEL:3:11296:3:8:1.00:0.50:00.27:0.35), (RABIES VIRUS STRAIN FLURY LEP ANTIGEN (PROPIOLACTONE INACTIVATED):3:11296:1:10:1.00:0.75:0.23:0.35), (YOHIMBINE:3:11296:1:10:1.00:0.75: 0.23:0.35), (ZOFENOPRIL CALCIUM:3:11296:1:10:1.00:0.75:0.23:0.35), (CHLOROPROCAINE:2:11297:2:9:1.00:0.50: 0.18:0.27), (CHONDROITIN SULFATE (BOVINE)HYALURONATE SODIUM:2:11297:2:9:1.00:0.50:0.18:0.27), (CITRIC ACID MONOHYDRATEMETHYLCELLULOSE:2:11297:1:10:1.00:0.67:0.17:0.27), (CLOPENTH IXOL HYDROCHLORIDE:2:11297:5:6:1.00:0.29:0.25:0.27), (COAGULATION FACTOR VIIA (RECOMBINANT):2:11297:2:9:1.00:0.50:0.18:0.27), (CUPRIC SULFATE ANHYDROUS:2:11297:2:9:1.00:0.50:0.18:0.27), (DEXTRAN 70GLYCERINHYPROMELLOSES:2:11297:6:5:1.00:0.25:0.29:0.27), (DIPHTHERIA AND TETANUS TOXOIDS AND POLIOVIRUS VACCINE ANTIGENS:2:11297:2:9:1.00:0.50:0.18:0.27), (LEVOLEUCOVORIN DISODIUM:2:11297:2:9:1.00:0.50:0.18:0.27), (ACETAMINOPHENDIPHENHYDRAMINE CITRATE:1:11298:2:9:1.00:0.33:0.10:0.15), (ARABICA COFFEE BEAN\ATROPA BELLADONNACALCIUM PHOSPHATECHAMOMILE:1:11298:3:8:1.00:0.25:0.11:0.15), (ERGOTAMINE TARTRATE:1:11298:3:8:1.00:0.25:0.11:0.15), (ERYTHROMYCIN ESTOLATE:1:11298:3:8:1.00:0.25:0.11:0.15), (FENRETINIDE:1:11298:3:8:1.00:0.25:0.11: 0.15), (HEPATITIS B VIRUS SUBTYPE ADW HBSAG SURFACE PROTEIN ANTIGEN:1:11298:0:11:1.00:1.00:0.08:0.15), (IOBENGUANE I-123:1:11298:2:9:1.00:0.33:0.10:0.15), (IOTHALAMATE MEGLUMINE:1:11298:1:10:1.00:0.50:0.09:0.15), (OXITROPIUM BROMIDE:1:11298:3:8:1.00:0.25:0.11:0.15), (SHEA BUTTER:1:11298:2:9:1.00:0.33:0.10:0.15), (ZOLEDRONATE DISODIUM:1:11298:4:7:1.00:0.20:0.13:0.15), (INFLUENZA A VIRUS A/CALIFORNIA/7/2009 (H1N1) RECOMBINANT HEMAGGLUTININ ANTIGEN\INFLUENZA A VIRUS A/VICTORIA/361/2011 (H3N2) RECOMBINANT HEMAGGLUTININ ANTIGEN\INFLUENZA B VIRUS B/WISCONSIN/1/2010 RECOMBINANT HEMAGGLUTININ ANTIGEN:0:11299:4:7:1.00:0.00:0.00:#NUM!), (MICONAZOLE NITRATE OR TERBINAFINE HYDROCHLORIDE OR TOLNAFTATE:0:11299:3:8:1.00:0.00:0.00:#NUM!), (ZINC ACETATE\ZINC GLUCONATE:0:11299:3:8:1.00:0.00:0.00:#NUM!), (COPPER:328:10970:0:12:1.00:1.00:0.96:0.98), (LAMIVUDINEZIDOVUDINE:239:11059:0:12:1.00:1.00:0.95:0.98), (CALCIUM CHLORIDEMAGNESIUM CHLORIDE\POTASSIUM CHLORIDE\SODIUM ACETATE\SODIUM CHLORIDE\SODIUM CITRATE:28:11270:0:12:1.00:1.00:0.70:0.82), (CALCIUM CHLORIDEDEXTROSEMAGNESIUM CHLORIDEOXIGLUTATIONE\POTASSIUM CHLORIDE\SODIUM BICARBONATE\SODIUM CHLORIDE\SODIUM PHOSPHATE\SODIUM PHOSPHATE, DIBASIC:9:11289:1:11:1.00:0.90:0.45:0.60), (HEMIN:9:11289:2:10:1.00:0.82:0.47:0.60), (CALCIUM CHLORIDEDEXTROSE MONOHYDRATEMAGNESIUM CHLORIDE\SODIUM BICARBONATE\SODIUM CHLORIDE\SODIUM LACTATE:6:11292:1:11:1.00:0.86:0.35:0.50), (INDORAMIN:5:11293:3:9:1.00:0.63:0.36:00.45), (NAPHAZOLINE NITRATEPREDNISOLONE:4:11294:1:11:1.00:0.80:0.27:0.40), (NIMETAZEPAM:4:11294: 3:9:1.00:0.57:0.31:0.40), (PHTHALYLSULFATHIAZOLE:4:11294:2:10:1.00:0.67:0.29:0.40), (PIPER METHYSTICUM ROOT:4:11294:2:10:1.00:0.67:0.29:0.40), (ZINC CHLORIDE:4:11294:0:12:1.00:1.00:0.25:0.40), (BENZOCAINELIDOCAINE HYDROCHLORIDE:3:11295:1:11:1.00:0.75:0.21:0.33), (PENTOBARBITAL SODIUM:3:11295:4:8:1.00:0.43:0.27:0.33), (TROFOSFAMIDE:3:11295:1:11:1.00:0.75:0.21: 0.33), (APIS MELLIFERA VENOM:2:11296:1:11:1.00:0.67:0.15:0.25), (AURANOFIN:2:11296:2:10:1.00:0.50:0.17:0.25), (CALCIUM CLMAGNESIUM CLPOTASSIUM CLSODIUM BICARSODIUM CLSODIUM PHOSPHATE,DIB:2:11296:2:10:1.00:0.50:0.17:0.25), (HUMAN ROTAVIRUS A TYPE G1P(8) STRAIN RIX4414 LIVE ANTIGEN:2:11296:1:11:1.00:0.67:0.15:0.25), (MELDONIUM:2:11296:4:8:1.00:0.33:0.20: 0.25), (NIMOTUZUMAB:2:11296:2:10:1.00:0.50:0.17:0.25), (NORGESTREL:2:11296:2:10: 1.00:0.50:0.17:0.25), (POLYLACTIDE, L-:2:11296:2:10:1.00:0.50:0.17:0.25), (TRYPAN BLUE:2:11296:1:11:1.00:0.67:0.15:0.25), (ATROPA BELLADONNACALCIUM PHOSPHATECHAMOMILECOFFEA ARABICA FRUIT:1:11297:3:9:1.00:0.25:0.10:0.14), (BENZOCAINEMENTHOL:1:11297:3:9:1.00:0.2: 0.10:0.14), (CAMPHOR (SYNTHETIC)MENTHOLMETHYL SALICYLATE:1:11297:2:10:1.00:0.33:0.09:0.14), (CEFATRIZINE:1:11297:4:8:1.00:0.20:0.11:0.14), (DEXTRANHYPROMELLOSES:1:11297:3:9:1.00:0.25:0.10:0.14), (ISOPROPYL ALCOHOLTENECTEPLASE:1:11297:3:9:1.00:0.25:0.10:0.14), (LEUCINE:1:11297:3:9:1.00:0.25:0.10:0.14), (PROGUANIL:1:11297:3:9:1.00:0.25:0.10:0.14), (SELINEXOR:1:11297: 3:9:1.00:0.25:0.10:0.14), (TAR:1:11297:6:6:1.00:0.14:0.14:0.14), (THROMBIN HUMAN:1:11297:3:9:1.00:0.25:0.10:0.14), (CALCIUM CHLORIDEDEXTROSE MONOHYDRATEHYDROCHLORIC ACIDMAGNESIUM CHLORIDE\SODIUM BICARBONATE\SODIUM CHLORIDE\SODIUM HYDROXIDE\SODIUM LACTATE:0:11298:7:5:1.00:0.00:0.00:#NUM!), (FECAL MICROBIOTA:0:11298:6:6:1.00:0.00:0.00:#NUM!), (IODINETOSITUMOMAB:0:11298:4:8: 1.00:0.00:0.00:#NUM!), (GADOTERIDOL:56:11241:4:9:1.00:0.93:0.86:0.90), (DIETHYLS TILBESTROL:8:11289:1:12:1.00:0.89:0.40:0.55), (RETEPLASE:8:11289:7:6:1.00:0.53:0.57: 0.55), (BROMISOVAL:5:11292:1:12:1.00:0.83:0.29:0.43), (VINFLUNINE:5:11292:0:13:1.00:1.00:0.28:0.43), (ANHYDROUS DEXTROSECALCIUM CHLORIDEMAGNESIUM CHLORIDE\SODIUM BICARBONATE\SODIUM CHLORIDE\SODIUM LACTATE:4:11293:2:11:1.00:0.67:0.27:0.38), (ANTHOXANTHUM ODORATUM POLLENDACTYLIS GLOMERATA POLLENLOLIUM PERENNE POLLENPRATENSE POLLENPOA PRATENSIS POLLEN:4:11293:2:11:1.00:0.67:0.27:0.38), (ANTIHEMOPHILIC FACTOR, PEGYLATED (MW 20000) HUMAN SEQUENCE RECOMBINANT:4:11293:0:13:1.00:1.00:0.24:0.38), (D+C YELLOW NO. 8:4:11293:0:13:1.00:1.00:0.24:0.38), (EDETIC ACID:4:11293:2:11:1.00:0.67:0.27:0.38), (ACETAMINOPHENCODEINE PHOSPHATEPROMETHAZINE HYDROCHLORIDE:3:11294:1:12:1.00:0.75:0.20:0.32), (AMILORIDE HYDROCHLORIDEHYDROCHLOROTHIAZIDETIMOLOL MALEATE:3:11294:2:11:1.00:0.60:0.21:0.32), (OMBITASVIR HEMINONAHYDRATEPARITAPREVIR DIHYDRATERITONAVIR:3:11294:6:7:1.00:0.33:0.30:0.32), (PHENYLALANINE, DL-:3:11294:2:11:1.00:0.60:0.21:0.32), (TECHNETIUM TC-99M TETROFOSMINTETROFOSMIN:3:11294:1:12:1.00:0.75:0.20:0.32), (AMDINOCILLIN:2:11295:0:13:1.00:1.00:0.13:0.24), (AMOXICILLINCLARITHROMYCINRABEPRAZOLE:2: 11295:1:12:1.00:0.67:0.14:0.24), (ASCORBIC ACIDNIACINAMIDEPYRIDOXINE\RIBOFLAVINTHIAMINE:2:11295:3:10:1.00:0.40:0.17:0.24), (BRIMONIDINE TARTRATEDORZOLAMIDE HYDROCHLORIDETIMOLOL MALEATE:2:11295:8:5:1.00:0.20:0.29:0.24), (CHOLINE SALICYLATE:2:11295:3:10:1.00:0.40:0.17:0.24), (CYCLOPENTOLATE HYDROCHLORIDEPHENYLEPHRINE HYDROCHLORIDE:2:11295:2:11:1.00:0.50:0.15:0.24), (DENILEUKIN DIFTITOX:2:11295:2:11:1.00:0.50:0.15:0.24), (DISODIUM FUMARATE:2:11295:3:10:1.00:0.40:0.17:0.24), (NETILMICIN:2:11295:2:11:1.00:0.50:0.1: 0.24), (RIFAMYCIN SODIUM:2:11295:1:12:1.00:0.67:0.14:0.24), (ACETAMINOPHENPSEUDOEPHEDRINE HYDROCHLORIDETRIPROLIDINE HYDROCHLORIDE:1:11296:1:12:1.00:0.50:0.08:0.13), (ANHYDROUS DEXTROSECALCIUM CHLORIDELACTIC ACIDMAGNESIUM CHLORIDE\POTASSIUM CHLORIDE\SODIUM BICARBONATE\SODIUM CHLORIDE:1:11296:3:10:1.00:0.25:0.09:0.13), (ANHYDROUS DEXTROSELACTIC ACIDMAGNESIUM CHLORIDE\POTASSIUM CHLORIDE\SODIUM BICARBONATE\SODIUM CHLORIDE:1:11296:2:11:1.00:0.33:0.08:0.13), (APATINIB:1:11296:3:10:1.00:0.25:0.09:0.13), (ASCORBIC ACIDCYANOCOBALAMINFOLIC ACIDIRON:1:11296:4:9:1.00:0.20:0.10:0.13), (CALCIUM CHLORIDEMAGNESIUM CHLORIDE\POTASSIUM CHLORIDE\SODIUM BICARBONATE\SODIUM CHLORIDE\SODIUM PHOSPHATE, DIBASIC:1:11296:3:10:1.00:0.25:0.09:0.13), (CENTELLA ASIATICA:1:11296:3:10:1.00:0.25:0.09:0.13), (COLISTIN A SODIUM METHANESULFONATE:1:11296:3:10:1.00:0.25:0.09:0.13), (DALBAVANCIN HYDROCHLORIDE:1:11296:3:10:1.00:0.25:0.09:0.13), (ERGONOVINE MALEATE:1:11296:3:10:1.00:0.25:0.09:0.13), (ESTRADIOL VALERATENORETHINDRONE:1:11296:3:10:1.00:0.25:0.09:0.13), (FOLLITROPINURO FOLLITROPIN:1:11296:0:13:1.00:1.00:0.07:0.13), (LANOLINMINERAL OIL:1:11296:6:7:1.00:0.14:0.13:0.13), (LIRILUMAB:1:11296:3:10:1.00:0.25:0.09:0.13), (M ECLIZINEPYRIDOXINE:1:11296:2:11:1.00:0.33:0.08:0.13), (NESIRITIDE:1:11296:3:10:1.00:0.25:0.09:0.13), (PAROMOMYCIN:1:11296:3:10:1.00:0.25:0.09:0.13), (SACROSIDAS E:1:11296:3:10:1.00:0.25:0.09:0.13), (SORAFENIB TOSYLATE:1:11296:4:9:1.00:0.20:0.10:0.13), (CIXUTUMUMAB:0:11297:4:9:1.00:0.00:0.00:#NUM!), (ONDANSETRON HYDROCHLORIDE:2584:8712:0:14:1.00:1.00:0.99:1.00), (INDINAVIR:21:11275:1:13:1.00: 0.95:0.62:0.75), (CETRORELIX ACETATE:17:11279:4:10:1.00:0.81:0.63:0.71), (DAUNORUBICIN CITRATE:8:11288:7:7:1.00:0.53:0.53:0.53), (FOLLITROPINLUTEINIZING HORMONE:7:11289:1:13:1.00:0.88:0.35:0.50), (ZUCLOPENTHIXOL HYDROCHLORIDE:7:11289:6:8:1.00:0.54:0.47:0.50), (ESTRADIOLNOMEGESTROL ACETATE:6:11290:1:13:1.00:0.86:0.32:0.46), (HYDROCHLOROTHIAZIDEMOEXIPRIL HYDROCHLORIDE:6:11290:1:13:1.00:0.86:0.32:0.46), (MEPTAZINOL HYDROCHLORIDE:6:11290:0:14:1.00:1.00:0.30:0.46), (CAFFEINEMAGNESIUM SALICYLATE:5:11291:5:9:1.00:0.50:0.36:0.42), (DEXTROSE\SODIUM BICARBONATE\SODIUM CHLORIDE\SODIUM LACTATE:5:11291:1:13:1.00:0.83:0.28:0.42), (ESTRAMUSTINE PHOSPHATE:5:11291:3:11:1.00:0.63:0.31:0.42), (ACETAMINOPHENASCORBIC ACIDPHENYLEPHRINE:4:11292:1:13:1.00:0.80:0.24:0.36), (AMYLMETACRESOLDICH LOROBENZYL ALCOHOL:4:11292:1:13:1.00:0.80:0.24:0.36), (BALSAM PERUCASTOR OILTRYPSIN:3:11293:4:10:1.00:0.43:0.23:0.30), (FALDAPREVIR:3:11293:3:11:1.00:0.50: 0.21:0.30), (FLUMETHASONE PIVALATE:3:11293:4:10:1.00:0.43:0.23:0.30), (SUCCINIC ACID:3:11293:0:14:1.00:1.00:0.18:0.30), (ACETAMINOPHENASCORBIC ACIDPHENYLEPHRINE HYDROCHLORIDE\SODIUM CITRATE:2:11294:1:13:1.00:0.67:0.13:0.22), (CERIVASTATIN:2:11294:4:10:1.00:0.33:0.17:0.22), (CHLORINE:2:11294:2:12:1.00:0.50:0.14:0.22), (CHROMIC CATIONCUPRIC CATIONFERROUS CATIONFLUORIDE IONIODIDE IONMANGANESE CATION (2+)SODIUM SELENIDE\ZINC CATION:2:11294:3:11:1.00:0.40:0.15:0.22), (CLEVIDIPINE:2:11294:2:12:1.00:0.50:0.14:00.22), (ETHAMBUTOL HYDROCHLORIDEISONIAZIDPYRAZINAMIDERIFAMYCIN:2:11294:2:12:1.00:0.50:0.14:0.22), (HELIUM:2:11294:2:12:1.00:0.50:0.14:0.22), (IRON ISOMALTOSIDE 1000:2:11294:2:12:1.00:0.50:0.14:0.22), (LEUPROLIDE ACETATENORETHINDRONE ACETATE:2:11294:4:10:1.00:0.33:0.17:0.22), (MALTODEXTRIN:2:11294:2:12:1.00:0.50: 0.14:0.22), (OCTINOXATETITANIUM DIOXIDE\ZINC OXIDE:2:11294:1:13:1.00:0.67:0.13:0.22), (PIXANTRONE:2:11294:2:12:1.00:0.50:0.14:0.22), (TENOFOVIR ALAFENAMIDE:2:11294:3:11:1.00:0.40:0.15:0.22), (TICARCILLIN:2:11294:5:9:1.00:0.29: 0.18:0.22), (BENOXINATE HYDROCHLORIDEFLUORESCEIN SODIUM:1:11295:1:13:1.00:0.50:0.07:0.13), (CYSTEAMINE HYDROCHLORIDE:1:11295:3:11:1.00:0.25:0.08:0.13), (INDIUM IN-111 CHLORIDEINDIUM IN-111 PENTETREOTIDEPENTETREOTIDE:1:11295:3:11:1.00:0.25:0.08:0.13), (MARIZOMIB:1: 11295:6:8:1.00:0.14:0.11:0.13), (MECHLORETHAMINE OXIDE HYDROCHLORIDE:1:11295:3:11:1.00:0.25:0.08:0.13), (CEP-9722:0:11296:4:10:1.00:0.00:0.00:#NUM!), (INIPARIB:0:11296:4:10:1.00:0.00:0.00:#NUM !), (VERUPREVIR:0:11296:4:10:1.00:0.00:0.00:#NUM!), (VEDOLIZUMAB:539:10756:0:15: 1.00:1.00:0.97:0.99), (ABATACEPT:523:10772:0:15:1.00:1.00:0.97:0.99), (DASABUVIR:396:10899:4:11:1.00:0.99:0.97:0.98), (ESLICARBAZEPINE:9:11286:5:10:1.00:0.64:0.47:0.55), (FIMASARTAN:9:11286:4:11:1.00:0.69:0.45:0.55), (CARBACHOL:8:11287:0:15:1.00:1.00:0.35:0.52), (LYMPHOCYTES:7:11288:6:9:1.00:0.54:0.44:0.48), (CALCIUMDEXTROS EMAGNESIUMSODIUM BICARBONATE\SODIUM CHLORIDE\SODIUM LACTATE:6:11289:0:15:1.00:1.00:0.29:0.44), (AMINOPYRINE:4:11291:1:14:1.00:0.80:0.22:0.35), (ANTIPYRINE:4:11291:0:15:1.00:1.00:0.21:0.35), (BENZYL BENZOATE:4:11291:0:15:1.00:1.00:0.21:0.35), (BUTOCONAZOLE NITRATE:4:11291:1:14:1.00:0.80:0.22:0.35), (CHROMIC CHLORIDECUPROUS CHLORIDEFERRIC CHLORIDEMANGANESE CHLORIDE\POTASSIUM IODIDE\SODIUM FLUORIDE\SODIUM MOLYBDATE DIHYDRATE\SODIUM SELENITE\ZINC CHLORIDE:4:11291:0:15:1.00:1.00:0.21:0.35), (ETHYLMORPHINE HYDROCHLORIDEHERBALS:4:11291:1:14:1.00:0.80:0.22:0.35), (FORMALDEHYDE:4:11291:5:10:1.00:0.44:0.29:0.35), (HYDROXYPROGESTERONE:4:11291:2:13:1.00:0.67:0.24:0.35), (PIMAVANSERIN:4:11291:4:11:1.00:0.50:0.27:0.35), (SODIUM HYDROXIDE:4:11291:7:8:1.00:0.36:0.33:0.35), (CALCIUM CHLORIDEMAGNESIUM CHLORIDE\SODIUM CHLORIDE\SODIUM LACTATE:3:11292:2:13:1.00:0.60:0.19:0.29), (DIACETYLMORPHINE HYDROCHLORIDE:3:11292:1:14:1.00:0.75:0.18:0.29), (EPHEDRA:3:11292:4:11:1.00:0.43: 0.21:0.29), (HUMAN VARICELLA-ZOSTER IMMUNE GLOBULIN:3:11292:3:12:1.00:0.50:0.20:0.29), (ISOPROPANOLAMINE:3:11292:0:15:1.00: 1.00:0.17:0.29), (NALMEFENE:3:11292:1:14:1.00:0.75:0.18:0.29), (SULPROSTONE:3:11292:1:14:1.00:0.75:0.18:0.29), (TETRAHYDROZOLINE HYDROCHLORIDE\ZINC SULFATE:3:11292:2:13:1.00:0.60:0.19:0.29), (ACETAMINOPHEN\CAFFEINEPYRILAMINE MALEATE:2:11293:1:14:1.00:0.67:0.13:0.21), (BROMPHENIRAMINE:2:11293:3:12:1.00:00.40:0.14:0.21), (CARBOMER HOMOPOLYMER TYPE C (ALLYL PENTAERYTHRITOL CROSSLINKED):2:11293:2:13:1.00:0.50:0.13:0.21), (MIVACURIUM CHLORIDE:2:11293:3:12:1.00:0.40:0.14:0.21), (MYCOPHENOLATE MOFETIL HYDROCHLORIDE:2:11293:2:13:1.00:0.50:0.13:0.21), (NOMEGESTROL:2:11293:3:12:1.0 0:0.40:0.14:0.21), (OREGANO:2:11293:4:11:1.00:0.33:0.15:0.21), (VANIPREVIR:2:11293: 2:13:1.00:0.50:0.13:0.21), (BUCLIZINE HYDROCHLORIDE:1:11294:3:12:1.00:0.25:0.08:0.12), (CITRIC ACID MONOHYDRATEDEXTROSE\SODIUM CITRATE\SODIUM PHOSPHATE, MONOBASIC:1:11294:3:12:1.00:0.25:0.08:0.12), (DIPHENHYDRAMINE HYDROCHLORIDE\ZINC ACETATE:1:11294:2:13:1.00:0.33:0.07:0.12), (HUMAN CLOSTRIDIUM TETANI TOXOID IMMUNE GLOBULIN:1:11294:3:12:1.00:0.25:0.08:0.12), (HYDROCORTISONEPRAMOXINE:1:112 94:5:10:1.00:0.17:0.09:0.12), (ILOPROST TROMETHAMINE:1:11294:3:12:1.00:0.25:0.08:0.12), (METHACHOLINE CHLORIDE:1:11294:5:10:1.00:0.17:0.09:0.12), (PHENETHICILLIN POTASSIUM:1:11294:3:12:1.00:0.25:0.08:0.12), (RADIUM:1:11294:9:6:1.00:0.10:0.14:0.1 2), (URELUMAB:1:11294:3:12:1.00:0.25:0.08:0.12), (ASPIRINDEXTROMETHORPHAN HYDROBROMIDEDOXYLAMINE SUCCINATEPHENYLEPHRINE BITARTRATE:0:11295:4:11:1.00:0.00:0.00:#NUM!), (HYALURONIDASE (HUMAN RECOMBINANT):0:11295:3:12:1.00:0.00:0.00:#NUM!), (PROPARACAINE:59:11235:10:6: 1.00:0.86:0.91:0.88), (EZOGABINE:36:11258:3:13:1.00:0.92:0.73:0.82), (MUROMONAB-CD3:16:11278:4:12:1.00:0.80:0.57:0.67), (CHLORPROPAMIDE:8:11286:3:13:1.00:0.73:0.3 8:0.50), (2-HYDROXYETHYL METHACRYLATE:6:11288:4:12:1.00:0.60:0.33:0.43), (FENOPROFEN:6:11288:7:9:1.00:0.46:0.40:0.43), (ACETAMINOPHEN\ASPIRIN\CAFFEINECODEINE PHOSPHATE:5:11289:5:11:1.00:0.50:0.31:0.38), (DEXTROSEHEPARIN:5:11289:7:9:1.00: 0.42:0.36:0.38), (KETAZOLAM:5:11289:2:14:1.00:0.71:0.26:0.38), (ACETAMINOPHENP ENTAZOCINE HYDROCHLORIDE:4:11290:0:16:1.00:1.00:0.20:0.33), (CHLOPHEDIANOL HYDROCHLORIDEDEXBROMPHENIRAMINE MALEATEPHENYLEPHRINE HYDROCHLORIDE:4:11290:12:4:1.00:0.25:0.50:0.33), (FARINAEDERMATOPHAGOIDES PTERONYSSINUS:4:11290:0:16:1.00:1.00:0.20:0.33), (FLUPIRTINE MALEATE:4:11290:1:15:1.00:0.80:0.21:0.33), (FUCUS VESICULOSUS:4:11290:1:15:1.00:0.80:0.21:0.33), (RUSCUS ACULEATUS ROOT:4:11290:3:13:1.00:0.57:0.24:0.33), (SAMARIUM SM-153 LEXIDRONAM PENTASODIUM:4:11290:8:8:1.00:0.33:0.33:0.33), (TECHNETIUM TC-99M SESTAMIBITETRAKIS(2-METHOXYISOBUTYLISOCYANIDE)COPPER(I) TETRAFLUOROBORATE:4:11290:1:15:1.00:0.80:0.21:0.33), (ALVIMOPAN:3:11291:1:15: 1.00:0.75:0.17:0.27), (ATROPINEDIPHENOXYLATE HYDROCHLORIDE:3:11291:3:13:1.00:0.50:0.19:0.27), (CAFFEINEERGOTAMINEERGO TAMINE TARTRATE:3:11291:1:15:1.00:0.75:0.17:0.27), (OMACETAXINE:3:11291:5:11:1.00:0.38: 0.21:0.27), (SODIUM SELENITE:3:11291:1:15:1.00:0.75:0.17:0.27), (TOLAZAMIDE:3:11291:1:15:1.00:0.75:0.1 7:0.27), (ACETAMINOPHENDICHLORALPHENAZONEISOMETHEPTENE MUCATE:2:11292:2:14:1.00:0.50:0.13:0.20), (AVOBENZONEOCTISALATEOCTOCRYL ENE:2:11292:1:15:1.00:0.67:0.12:0.20), (CALCIUM MONOETHYLFUMARATEDIMETHYL FUMARATEMAGNESIUM MONOETHYLFUMARATE\ZINC MONOETHYLFUMARATE:2:11292:2:14:1.00:0.50:0.13:0.20), (COPPERINTRAUTERINE DEVICE:2:11292:5:11:1.00:0.29:0.15:0.20), (FENTANYL HYDROCHLORIDE:2:11292:2:14:1.00:0.50:0.13:0.20), (FERROUS FUMARATEMESTRANOLNORETHINDRONE:2:11292:2:14:1.00:0.50:0.13:0.20), (HYD ROCHLOROTHIAZIDEZOFENOPRIL:2:11292:2:14:1.00:0.50:0.13:0.20), (MELITRACEN: 2:11292:2:14:1.00:0.50:0.13:0.20), (METFORMINSITAGLIPTIN PHOSPHATE:2:11292:2:14:1.00:0.50:0.13:0.20), (POTASSIUM HYDROXIDE\POTASSIUM PHOSPHATE, DIBASIC\SODIUM PHOSPHATE, DIBASIC, ANHYDROUS:2:11292:2:14:1.00:0.50:0.13:0.20), (RUXOLITINIB PHOSPHATE:2:11292:4:12:1.00:0.33:0.14:0.20), (SINCALIDE:2:11292:2:14:1.00:0.50:0.13: 0.20), (SUCCIMER:2:11292:2:14:1.00:0.50:0.13:0.20), (TREBANANIB:2:11292:2:14:1.00: 0.50:0.13:0.20), (ALITRETINOIN:1:11293:2:14:1.00:0.33:0.07:0.11), (HYALURONIDASE, OVINE:1:11293:1:15:1.00:0.50:0.06:0.11), (METFORMIN HYDROCHLORIDEPIOGLITAZONE HYDROCHLORIDE:170:11123:0:17:1.00:1.00:0.91:0.95), (FENOTEROL:127:11166:0:17:1.00:1.00:0.88:0.94), (DIENOGESTESTRADIOL VALERATE:102:11191:0:17:1.00:1.00:0.86:0.92), (STANNOUS FLUORIDE:18:11275:2:15:1.00:0.90:0.55:0.68), (ENALAPRIL MALEATENITRENDIPINE:8:11285:1:16:1.00:0.89:0.33:0.48), (CANGRELOR:7:11286:3:1 4:1.00:0.70:0.33:0.45), (OXETORONE:7:11286:8:9:1.00:0.47:0.44:0.45), (COAGULATION FACTOR IX HUMAN:6:11287:0:17:1.00:1.00:0.26:0.41), (COAGULATION FACTOR IX RECOMBINANT HUMAN:6:11287:4:13:1.00:0.60:0.32:0.41), (LEVOMEPROMAZINE HYDROCHLORIDELEVOMEPROMAZINE MALEATE:6:11287:6:11:1.00:0.50:0.35:0.41), (MAGNESIUM LACTATEPYRIDOXINE HYDROCHLORIDE:6:11287:2:15:1.00:0.75:0.29:0.41), (CHLORAMPHENICOLPREDNIS OLONE:5:11288:5:12:1.00:0.50:0.29:0.37), (CHLORPROMAZINE HIBENZATE:5:11288:3:14:1.00:0.63:0.26:0.37), (HYDROCODONE BITARTRATEPHENYLPROPANOLAMINE HYDROCHLORIDE:5:11288:2:15:1.00:0.71:0.25:0.37), (ZICONOTIDE:5:11288:1:16:1.00:0 0.83:0.24:0.37), (ACETYLDIGITOXIN:4:11289:2:15:1.00:0.67:0.21:0.32), (ASCORBIC ACIDCHONDROITIN SULFATE (BOVINE)GLUCOSAMINE SULFATEMANGANESE:4:11289:9:8:1.00:0.31:0.33:0.32), (CEPHALOTHIN SODIUM:4:11289:1:16:1.00:0.80:0.20:0.32), (CHOLINE BITARTRATE:4:11289:7:10:1.00:0.36:0.29:0.32), (RIBOFLAVIN 5′-PHOSPHATE SODIUM:4:11289:6:11:1.00:0.40:0.27:0.32), (TACALCITOL:4:11289:0:17:1.00:1.00:0.19: 0.32), (TRIENTINE:4:11289:5:12:1.00:0.44:0.25:0.32), (AMPICILLIN TRIHYDRATE:3:11290:2:15:1.00:0.60:0.17:0.26), (ARBEKACIN:3:11290:1:16:1.00:0.75:0 0.16:0.26), (ASPIRIN\CAFFEINEPHENACETIN:3:11290:1:16:1.00:0.75:0.16:0.26), (ASPIR IN\SODIUM BICARBONATE:3:11290:2:15:1.00:0.60:0.17:0.26), (AVOBENZONEOCTISALATEOCT OCRYLENEOXYBENZONE:3:11290:0:17:1.00:1.00:0.15:0.26), (BRODALUMAB:3:11290: 6:11:1.00:0.33:0.21:0.26), (EFAVIRENZLAMIVUDINETENOFOVIR:3:11290:0:17:1.00:1 0.00:0.15:0.26), (FLUTICASONE FUROATEUMECLIDINIUM BROMIDEVILANTEROL TRIFENATATE:3:11290:1:16:1.00:0.75:0.16:0.26), (GOTU KOLA:3:11290:6:11:1.00:0.33:0.21:0.26), (PROPOXYPHENE NAPSYLATE:3:11290:6:11:1.00:0.33:0.21:0.26), (SALICYLIC ACIDTRIAMCINOLONE ACETONIDE:3:11290:2:15:1.00:0.60:0.17:0.26), (AMINOSALICYLATE SODIUM:2:11291:2:15:1.00:0.50:0.12:0.19), (ASPIRIN SODIUM:2:11291:2:15:1.00:0.50:0.12:0.19), (ATAZANAVIR SULFATECOBICISTAT:2:11291:0:17:1.00:1.00:0.11:0.19), (BROMPHENIRAMINE MALEATEDEXTROMETHORPHAN HYDROBROMIDEPHENYLEPHRINE HYDROCHLORIDE:2:11291:2:15:1.00:0.50:0.12:0.19), (CARVEDILOL HYDROCHLORIDE:2:11291:2:15:1.00:0.50:0.12:0.19), (ETHAMBUTOLISONIAZIDPYR AZINAMIDERIFAMYCIN:2:11291:2:15:1.00:0.50:0.12:0.19), (FLUOXYMESTERONE:2:1 1291:3:14:1.00:0.40:0.13:0.19), (GLYCERINPETROLATUMPHENYLEPHRINE HYDROCHLORIDEPRAMOXINE HYDROCHLORIDE:2:11291:2:15:1.00:0.50:0.12:0.19), (MECHLORETHAMINE HYDROCHLORIDE:2:11291:2:15:1.00:0.50:0.12:0.19), (METYROSINE:2:11291:8:9:1.00:0 0.20:0.18:0.19), (NEOMYCIN SULFATEPOLYMYXIN B SULFATE:2:11291:1:16:1.00:0.67:0.11:0.19), (OLSALAZINE SODIUM:2:11291:4:13:1.00:0.33:0.13:0.19), (UNOPROSTONE ISOPROPYL:2:11291:1:16:1.00:0.67:0.11:0.19), (AMMONIA:1:11292:2:15:1.00:0.33:0.06: 0.11), (CALCIUM CARBONATEDIMETHICONE:1:11292:5:12:1.00:0.17:0.08:0.11), (CALCIUM CHLORIDE\POTASSIUM CHLORIDE\SODIUM LACTATE:1:11292:2:15:1.00:0.33:0.06:0.11), (CIPROFLOXACINFLUOCINOLONE ACETONIDE:1:11292:2:15:1.00:0.33:0.06:0.11), (METHSCOPOLAMINEMETHSCOPOLA MINE BROMIDE:1:11292:3:14:1.00:0.25:0.07:0.11), (MONOSODIUM GLUTAMATE:1:11292:4:13:1.00:0.20:0.07:0.11), (PYRANTEL PAMOATE:1:11292:2:15:1.00:0.33:0.06:0.11), (TECHNETIUM TC-99M SULFUR COLLOID:1:11292:1:16:1.00:0.50:0.06:0.11), (TEDIZOLID:1:11292:6:11:1.00:0.14:0.08:0.11), (TITANIUM DIOXIDE:1:11292:1:16:1.00:0.50:0.06:0.11), (URIDINE TRIACETATE:0:11293:4:13:1.00:0.00:0.00:#NUM!), ((1-743)-(1638-2332)-BLOOD-COAGULATION FACTOR VIII (SYNTHETIC HUMAN) FUSION PROTEIN WITH IMMUNOGLOBULIN G1 (SYNTHETIC HUMAN FC DOMAIN FRAGMENT), (1444-6′), (1447-9′)-BIS(DISULFIDE) WITH IMMUNOGLOBULIN G1 (SYNTHETIC HUMAN FC DOMAIN FRAGMENT):101:11191:1:17:1.00:0.99:0.86:0.92), (.ALPHA.-TOCOPHEROLASCORBIC ACIDBIOTINCALCIUM CARBONATECHOLECALCIFEROLCOPPERCYANOCOBALAMINFERROUS FUMARATEFOLIC ACIDIODINEMAGNESIUMNIACINAMIDEPANTOTHENIC ACIDPYRIDOXINE\RIBOFLAVINTHIAMINEVITAMIN A\ZINC:12:11280:2:16:1.00:0.86:0.43:0.57), (HERBALS\VITIS VINIFERA SEED:9:11283:12:6:1.00:0.43:0.60:0.50), (ANTHRALIN:6:11286:1:17:1.00:0.86:0.26:0.40), (ELVITEGRAVIR:5:11287:1:17:1.00:0.83:0.23:0.36), (ASPIRIN\CAFFEINEDIHYDROC ODEINE BITARTRATE:4:11288:0:18:1.00:1.00:0.18:0.31), (CARBOPROST TROMETHAMINE:4:11288:3:15:1.00:0.57:0.21:0.31), (EZETIMIBEROSUVASTATIN:4:1 1288:4:14:1.00:0.50:0.22:0.31), (FLUPENTIXOL DIHYDROCHLORIDE:4:11288:12:6:1.00:0.25:0.40:0.31), (MIRABEGRONSOLIFENACIN: 4:11288:1:17:1.00:0.80:0.19:0.31), (NERATINIB:4:11288:2:16:1.00:0.67:0.20:0.31), (POT ASSIUM BITARTRATE:4:11288:14:4:1.00:0.22:0.50:0.31), (ACETIC ACIDSODIUM ACETATE:3:11289:9:9:1.00:0.25:0.25:0.25), (ALVERINE:3:11289:0:18:1.00:1.00:0.14:0.2), (BENZYL ALCOHOL:3:11289:1:17:1.00:0.75:0.15:0.25), (ESTRADIOL HEMIHYDRATENORETHINDRONE ACETATE:3:11289:6:12:1.00:0.33:0.20:0.25), (ETELCALCETIDE:3:11289:1:17:1.00:0.75 0.15:0.25), (OATMEAL:3:11289:0:18:1.00:1.00:0.14:0.25), (SULFAMETHIZOLE:3:11289:0: 18:1.00:1.00:0.14:0.25), (CELERY SEED:2:11290:6:12:1.00:0.25:0.14:0.18), (ENASIDENIB MESYLATE:2:11290:3:15:1.00:0.40:0.12:0.18), (MAGNESIUM TRISILICATE:2:11290:2:16:1.00:0.50:0.11:0.18), (METHYSERGIDE MALEATE:2:11290:5:13:1.00:0.29:0.13:0.18), (MEPHENESIN:1:11291:3:15:1.00:0.25:0.06: 0.10), (METHOTREXATEMETHOTREXATE SODIUM:1:11291:14:4:1.00:0.07:0.20:0.10), (OTERACIL:1:11291:3:15:1.00:0.25:0.06:0.1 0), (PLATELET CONCENTRATE HUMAN:1:11291:2:16:1.00:0.33:0.06:0.10), (CAMPHORATED PHENOL:0:11292:4:14:1.00:0.00:0.00:#NUM!), (ROMOSOZUMAB:0:11292:5:13:1.00:0.00: 0.00:#NUM!), (MAGNESIUM SALICYLATE:11:11280:0:19:1.00:1.00:0.37:0.54), (CLOFIBRATE:8:11283:0:19:1.00:1.00: 0.30:0.46), (SODIUM FLUORIDETRICLOSAN:7:11284:3:16:1.00:0.70:0.30:0.42), (PIRMENOL HYDROCHLORIDE:6:11285:4:15:1.00:0.60:0.29:0.39), (ACETAMINOPHENPROPOXYP HENE:5:11286:0:19:1.00:1.00:0.21:0.34), (TROPINE BENZYLATE:5:11286:5:14:1.00:0.50:0.26:0.34), (BUPRENORPHINE HYDROCHLORIDENALOXONE HYDROCHLORIDE DIHYDRATE:4:11287:2:17:1.00:0.67:0.19:0.30), (CHLORHEXIDINE GLUCONATELIDOCAINE HYDROCHLORIDE:4:11287:1:18:1.00:0.80:0.18:0.30), (ISOCARBOXAZID:4:11287:5:14:1 0.00:0.44:0.22:0.30), (BACILLUS CALMETTE-GUERIN SUBSTRAIN CONNAUGHT LIVE ANTIGEN:3:11288:11:8:1.00:0.21:0.27:0.24), (BENZPHETAMINE HYDROCHLORIDE:3:11288:3:16:1.00:0.50:0.16:0.24), (DIATRIZOATE MEGLUMINE:3:11288:1:18:1.00:0.75:0.14:0.24), (ESTROGENS, CONJUGATEDNORGESTREL:3:11288:2:17:1.00:0.60:0.15:0.24), (HYALURONIDASE (HUMAN RECOMBINANT)RITUXIMAB:3:11288:2:17:1.00:0.60:0.15:0.24), (LANINAMIVIR:3:112 88:4:15:1.00:0.43:0.17:0.24), (MAZINDOL:3:11288:2:17:1.00:0.60:0.15:0.24), (NAPHAZO LINENAPHAZOLINE HYDROCHLORIDE:3:11288:1:18:1.00:0.75:0.14:0.24), (PHENOXYBENZAMINE:3:11288: 5:14:1.00:0.38:0.18:0.24), (PIPERONYL BUTOXIDEPYRETHRUM EXTRACT:3:11288:0:19:1.00:1.00:0.14:0.24), (PROTEIN C:3:11288:1:18:1.00:0.75:0.14:0.24), (UPROSERTIB:3:11288:1:18:1.00:0.75:0.14:0.24), (. ALPHA.-TOCOPHEROL ACETATE, D-ASCORBIC ACIDCALCIUM PANTOTHENATEFOLIC ACIDMANGANESENIACINAMIDEPYRIDOXINE\RIBOFLAVINTHIAMINEVITAMIN A\ZINC OXIDE:2:11289:13:6:1.00:0.13:0.25:0.17), (ACIPIMOX:2:11289:2:17:1.00:0.50:0.11:0.17), (ALANINEAMINO ACIDSARGININECALCIUM CHLORIDEDEXTROSEGLYCINEHISTIDINEISOLEUCINELEUCINELYSINEMAGNE SIUM CHLORIDEMETHIONINEPHENYLALANINE\POTASSIUM PHOSPHATE, DIBASICPROLINEPROLINE, DL-SERINE\SODIUM ACETATE\SODIUM CHLORIDETHREONINETRYPTOPHANTYROSINEVALINE:2:11289:1:18:1.00:0.67:0.1 0:0.17), (BACITRACIN ZINCNEOMYCIN SULFATEPOLYMYXIN B SULFATEPRAMOXINE HYDROCHLORIDE:2:11289:1:18:1.00:0.67:0.10:0.17), (CYTARABINEDAUNORUBICIN: 2:11289:2:17:1.00:0.50:0.11:0.17), (DEXTROSEFRUCTOSETRISODIUM CITRATE DIHYDRATE:2:11289:2:17:1.00:0.50:0.11:0.17), (DEXTROSELIDOCAINE HYDROCHLORIDE:2:11289:2:17:1.00:0.50:0.11:0.17), (ETEPLIRSEN:2:11289:10:9:1.00:0 0.17:0.18:0.17), (FLUDEOXYGLUCOSE F-18:2:11289:3:16:1.00:0.40:0.11:0.17), (MAGNESIUM STEARATE:2:11289:5:14:1.00:0.29:0.13:0.17), (ACETOHYDROXAMIC ACID:1:11290:9:10:1.00:0.10:0.09:0.10), (APROTININ:1:11290:3:16:1.00:0.25:0.06:0.10), (ISOPROPYL MYRISTATE:1:11290:3:16:1.00:0.25:0.06:0.10), (MAGNESIUM ALUMINUM SILICATE:1:11290:4:15:1.00:0.20:0.06:0.10), (PEGINTERFERON ALFA-2BRIBAVIRIN:1:11290:4:15:1.00:0.20:0.06:0.10), (STARCH, CORN:1:11290:3:16:1.00:0.25:0.06:0.10), (TECHNETIUM TC-99M SODIUM PERTECHNETATE:1:11290:1:18:1.00:0.50:0.05:0.10), (ANHYDROUS CITRIC ACIDPOTASSIUM BICARBONATE\SODIUM BICARBONATE:0:11291:3:16:1.00:0.00:0.00:#NUM!), (EQUISETUM ARVENSE TOP:0:11291:4:15:1.00:0.00:0.00:#NUM!), (RILOTUMUMAB:0:11291:9:10:1.00:0.00:0.00: #NUM!), (TRANYLCYPROMINE:18:11272:6:14:1.00:0.75:0.56:0.64), (ESTRONE:10:11280: 9:11:1.00:0.53:0.48:0.50), (CLONIXIN:7:11283:0:20:1.00:1.00:0.26:0.41), (COLCHICINE OPIUMTIEMONIUM METHYLSULFATE:6:11284:4:16:1.00:0.60:0.27:0.38), (INVESTIGATIONAL BIOSIMILARS:6:11284:2:18:1.00:0.75:0.25:0.38), (TOREMIFENE:6:11284:9:11:1.00:0.40: 0.35:0.38), (TRIMETHADIONE:6:11284:3:17:1.00:0.67:0.26:0.38), (ALPHA-TOCOPHEROLASCORBIC ACIDCHOLECALCIFEROLCOPPERCYANOCOBALAMINFOLIC ACIDLUTEINOMEGA-3 FATTY ACIDSPYRIDOXINE HYDROCHLORIDETAURINEVITAMIN A PALMITATEZEAXANTHINZINC PICOLINATE:5:11285:9:11:1.00:0.36:0.31:0.33), (AMPHETAMINE ASPARTATEDEXTROAMPHETAMINE SACCHARATE:5:11285:8:12:1.00:0.38:0.29:0.33), (INTERFERON ALFACON-1:5:11285:6:14:1.00:0.45:0.26:0.33), (ANTIPYRINEBENZOCAINE:4:11286:2:18:1.00:0.67: 0.18:0.29), (BORNAPRINE HYDROCHLORIDE:4:11286:6:14:1.00:0.40:0.22:0.29), (BUDESONIDEINDACATEROL MALEATE:4:11286:9:11:1.00:0.31:0.27:0.29), (CAMPTOTHECIN:4:11286:1:19:1.00:0.80: 0.17:0.29), (CLORAZEPIC ACID:4:11286:3:17:1.00:0.57:0.19:0.29), (CODEINE PHOSPHATEIBUPROFEN:4:11286:1:19:1.00:0.80:0.17:0.29), (LEVOMEFOLATE CALCIUM\METHYLCOBALAMINPYRIDOXAL 5-PHOSPHATE:4:11286:16:4:1.00:0.20:0.50:0.29), (PASSIFLORA INCARNATA FLOWER:4:11286:10:10:1.00:0.29:0.29:0.29), (SILICON:4:11286:2:18:1.00:0.67:0.18:0.29), (TRYPSIN:4:11286:3:17:1.00:0.57:0.19:0.29), (VARLILUMAB:4:11286:0:20:1.00:1.00:0.17:0.29), (.ALPHA.-TOCOPHEROLASCORBIC ACID:3:11287:2:18:1.00:0.60:0.14:0.23), (ACETAMINOPHENBUCLIZINE HYDROCHLORIDECODEINE:3:11287:5:15:1.00:0.38:0.17:0.23), (ACTIVATED CHARCOALDIMETHICONE:3:11287:1:19:1.00:0.75:0.14:0.23), (CHLORPHENIRAMINE MALEATEHYDROCODONE BITARTRATEPSEUDOEPHEDRINE HYDROCHLORIDE:3:11287:10:10:1.00:0.23:0.23:0.23), (CITRIC ACID MONOHYDRATE\SODIUM CITRATE:3:11287:1:19:1.00:0.75:0.14:0.23), (DASABUVIR SODIUM MONOHYDRATE:3:11287:7:13:1.00:0.30:0.19:0.23), (FERROUS FUMARATEFOLIC ACID:3:11287:0:20:1.00:1.00:0.13:0.23), (GRAPEFRUIT JUICE:3:11287:2:18:1.00:0.60:0.14:0.23), (METHOHEXITAL:3:11287:2:18:1.00:0.60:0.14: 0.23), (PENTETRAZOL:3:11287:5:15:1.00:0.38:0.17:0.23), (PHENAGLYCODOL:3:11287:1: 19:1.00:0.75:0.14:0.23), (POTASSIUM BICARBONATE\POTASSIUM CITRATE:3:11287:4:16:1.00:0.43:0.16:0.23), (PRILOCAINE HYDROCHLORIDE:3:11287:1:19:1.00:0.75:0.14:0.23), (SERENOA REPENS WHOLE:3:11287:1:19:1.00:0.75:0.14:0.23), (STRONTIUM CHLORIDE SR-89:3:11287:2:18:1.00:0.60:0.14:0.23), (ALANINEARGININEDEXTROSEGLYCINEHIST IDINEISOLEUCINELEUCINELYSINEMETHIONINEPHENYLALANINEPROLINESE RINETHREONINETRYPTOPHANTYROSINEVALINE:2:11288:0:20:1.00:1.00:0.09:0.17), (AMRUBICIN:2:11288:2:18:1.00:0.50:0.10:0.17), (BAMBUTEROL:2:11288:2:18:1.00:0.5 0:0.10:0.17), (CEPHALEXIN ANHYDROUS:2:11288:2:18:1.00:0.50:0.10:0.17), (CLENBUTEROL:2:11288:2:18:1.00:0.5 0:0.10:0.17), (GRAPEFRUIT SEED EXTRACT:2:11288:8:12:1.00:0.20:0.14:0.17), (HEPARIN SODIUMSODIUM CHLORIDE:2:11288:4:16:1.00:0.33:0.11:0.17), (SESAME OIL:2:11288:4:16:1.00:0.33:0.11:0.17), (YELLOW FEVER VIRUS STRAIN 17D-204 LIVE ANTIGEN:2:11288:1:19:1.00:0.67:0.10:0.17), (AMPHETAMINE ADIPATEDEXTROAMPHETAMINE:1:11289:4:16:1.00:0.20:0.06:0.09), (ASPIRINCALCI UM CARBONATE:1:11289:2:18:1.00:0.33:0.05:0.09), (BOTULISM ANTITOXIN:1:11289:19:1:1.00:0.05:0.50:0.09), (DOCETAXEL ANHYDROUS:1:11289:3:17:1.00:0.25:0.06:0.09), (EPHEDRINE SULFATE:1:11289:3:17:1.00:0.25:0.06:0.09), (IDEBENONE:1:11289:3:17:1.00:0.25:0.06:0 0.09), (LETROZOLERIBOCICLIB:1:11289:17:3:1.00:0.06:0.25:0.09), (MEROPENEM ANHYDROUS:1:11289:3:17:1.00:0.25:0.06:0.09), (SODIUM TETRADECYL SULFATE:1:11289:0:20:1.00:1.00:0.05:0.09), (PROPARACAINE HYDROCHLORIDE:71:11218:0:21:1.00:1.00:0.77:0.87), (ALISKIREN HEMIFUMARATEVALSARTAN:15:11274:13:8:1.00:0.54:0.65:0.59), (PERGOLIDE:15:11 274:4:17:1.00:0.79:0.47:0.59), (BIPERIDEN LACTATE:9:11280:6:15:1.00:0.60:0.38:0.46), (ACETAMINOPHEN\CAFFEINEDIHYDRO CODEINE BITARTRATE:7:11282:1:20:1.00:0.88:0.26:0.40), (AMINOLEVULINIC ACID HYDROCHLORIDE:7:11282:1:20:1.00:0.88:0.26:0.40), (DILAZEP DIHYDROCHLORIDE:7:11282:5:16:1.00:0.58:0.30:0.40), (PROSTATE:7:11282:3:18:1.00: 0.70:0.28:0.40), (BENFLUOREX:6:11283:4:17:1.00:0.60:0.26:0.36), (LEVORPHANOL:6:1 1283:4:17:1.00:0.60:0.26:0.36), (LYSERGIDE:6:11283:7:14:1.00:0.46:0.30:0.36), (.ALPHA.-TOCOPHEROLASCORBIC ACIDCALCIUM CITRATECHOLECALCIFEROLCOPPERDOCONEXENTDOCUSATE SODIUMFOLIC ACIDICOSAPENTIODINEIRONNIACINAMIDEPYRIDOXINE HYDROCHLORIDE\RIBOFLAVINTHIAMINE\ZINC:5:11284:1:20:1.00:0.83:0.20:0.32), (BENRALIZUMAB:5:11284:6:15:1.00:0.45:0.25:0.32), (FERRIC SULFATE:5:11284:3:18:1.00:0.63:0.22:0.32), (GINSENG EXTRACT:5:11284:8:13:1.00:0.38:0.28:0.32), (LITHIUM OROTATE:5:11284:6:15:1.00:0.45:0.25:0.32), (METHYCLOTHIAZIDE:5:11284:3:18:1.00: 0.63:0.22:0.32), (PICOLAMINE SALICYLATE:5:11284:3:18:1.00:0.63:0.22:0.32), (RILMAZAFONE:5:11284:6:15:1.00:0.45: 0.25:0.32), (STIRIPENTOL:5:11284:3:18:1.00:0.63:0.22:0.32), (SUCROSE:5:11284:0:21:1.00:1.00:0.19:0.32), (AMINOBENZOIC ACID:4:11285:0:21:1.00:1.00:0.16:0.28), (ANGIOTENSIN II:4:11285:7:14:1.00:0.36:0.22:0.28), (BUPIVACAINE HYDROCHLORIDEEPINEPHRINE:4:11285:0:21:1.00:1.00:0.16:0.28), (CILAZAPRIL ANHYDROUSHYDROCHLOROTHIAZIDE:4:11285:2:19:1.00:0.67:0.17:0.28), (DIHYDR OTACHYSTEROL:4:11285:1:20:1.00:0.80:0.17:0.28), (DOXIFLURIDINE:4:11285:7:14:1.0 0:0.36:0.22:0.28), (SAFINAMIDE:4:11285:17:4:1.00:0.19:0.50:0.28), (TEMOCAPRIL:4:11 285:11:10:1.00:0.27:0.29:0.28), (TRIBASIC CALCIUM PHOSPHATE:4:11285:1:20:1.00:0.80:0.17:0.28), (ACETAMINOPHENDEXTROMETHOR PHAN HYDROBROMIDEPSEUDOEPHEDRINE HYDROCHLORIDE:3:11286:1:20:1.00:0.75:0.13:0.22), (ACETAMINOPHENPSEUDOEPH EDRINE:3:11286:3:18:1.00:0.50:0.14:0.22), (ALLERGENIC EXTRACTS:3:11286:0:21:1.00:1.00:0.13:0.22), (AVOBENZONEHOMOSALATEOCTISA LATEOCTOCRYLENE:3:11286:0:21:1.00:1.00:0.13:0.22), (INDISETRON HYDROCHLORIDE:3:11286:4:17:1.00:0.43:0.15:0.22), (ISOLEUCINE:3:11286:3:18:1.00:0 0.50:0.14:0.22), (RIFAPENTINE:3:11286:1:20:1.00:0.75:0.13:0.22), (TISAGENLECLEUCEL-T:3:11286:1:20:1.00:0.75:0.13:0.22), (ACYCLOVIRHYDROCORTISONE:2:11287:2:19:1.0 0:0.50:0.10:0.16), (ALLANTOIN:2:11287:3:18:1.00:0.40:0.10:0.16), (BUSERELIN ACETATE:2:11287:2:19:1.00:0.50:0.10:0.16), (CARBOPLATINPACLITAXEL:2:11287:5:1 6:1.00:0.29:0.11:0.16), (ESTROGENSPROGESTERONE:2:11287:2:19:1.00:0.50:0.10:0.16), (HYDROCODONEHYDROCODONE BITARTRATEIBUPROFEN:2:11287:3:18:1.00:0.40:0.10:0.16), (MENADIONE:2:11287:3:1 8:1.00:0.40:0.10:0.16), (OXYMETAZOLINE HYDROCHLORIDEPSEUDOEPHEDRINE HYDROCHLORIDE:2:11287:4:17:1.00:0.33:0.11:0.16), (WINE:2:11287:2:19:1.00:0.50:0.1 0:0.16), (ALISERTIB:1:11288:3:18:1.00:0.25:0.05:0.09), (CALCIUM CHLORIDEDEXTROSE MONOHYDRATE\POTASSIUM CHLORIDE\SODIUM CHLORIDE\SODIUM LACTATE:1:11288:1:20:1.00:0.50:0.05:0.09), (DIPHTHERIA AND TETANUS TOXOIDS AND ACELLULAR PERTUSSIS ADSORBED, INACTIVATED POLIOVIRUS AND HAEMOPHILUS B CONJUGATE (TETANUS TOXOID CONJUGATE) VACCINE:1:11288:0:21:1.00:1.00:0.05:0.09), (HEXAMIDINE DIISETHIONATE:1:11288:3:18:1.00:0.25:0.05:0.09), (ISOSULFAN BLUE:1:11288:2:19:1.00:0.33:0.05:0.09), (METHYLPREDNISOLONE:2620:8668:0:22:1.00: 1.00:0.99:1.00), (AMINO ACIDSCALCIUM CHLORIDEMAGNESIUM CHLORIDE\SODIUM CHLORIDE\SODIUM LACTATE:9:11279:3:19:1.00:0.75:0.32:0.45), (ACETAMINOPHENCHLORPHENIRAMIN E MALEATEDEXTROMETHORPHAN HYDROBROMIDEPHENYLEPHRINE HYDROCHLORIDE OR ACETAMINOPHENDEXTROMETHORPHAN HYDROBROMIDEDOXYLAMINE SUCCINATEPHENYLEPHRINE HYDROCHLORIDEOR HOMEOPATHICS:6:11282:5:17:1.00:0.55:0.26:0.35), (AMLODIPINEINDAPAMIDE:5:112 83:2:20:1.00:0.71:0.20:0.31), (DYCLONINE:5:11283:2:20:1.00:0.71:0.20:0.31), (LACTOB ACILLUS ACIDOPHILUSSTREPTOCOCCUS FAECAIS:5:11283:2:20:1.00:0.71:0.20:0.31), (MAGNESIUM GLUCEPTATE:5:11283:11:11:1.00:0.31:0.31:0.31), (MAMMAL LIVER:5:11283:0:22:1.00:1.00:0.19:0.31), (PHENYLEPHRINE HYDROCHLORIDEPREDNISOLONE ACETATE:5:11283:1:21:1.00:0.83:0.19:0.31), (ALUMINUM CHLOROHYDRATE:4:11284:1:21:1.00:0.80:0.16:0.27), (FERROUS SULFATESERINE:4:11284:0:22:1.00:1.00:0.15:0.27), (HEPATITIS B VIRUS HBSAG SURFACE PROTEIN ANTIGEN:4:11284:1:21:1.00:0.80:0.16:0.27), (SOLIFENACINTAMSULOSIN:4:11284:5:17: 1.00:0.44:0.19:0.27), (BACITRACINNEOMYCIN SULFATEPOLYMYXIN B SULFATE:3:11285:14:8:1.00:0.18:0.27:0.21), (BAZEDOXIFENE ACETATE:3:11285:1:21:1.00:0.75:0.13:0.21), (DIETHYLPROPION HYDROCHLORIDE:3:11285:0:22:1.00:1.00:0.12:0.21), (ERGOCALCIFEROLPHYTONAD IONERETINOLTOCOPHEROL:3:11285:1:21:1.00:0.75:0.13:0.21), (HISTAMINE:3:11285: 1:21:1.00:0.75:0.13:0.21), (NIMUSTINE:3:11285:5:17:1.00:0.38:0.15:0.21), (PIZOTYLINE MALEATE:3:11285:2:20:1.00:0.60:0.13:0.21), (BERACTANT:2:11286:2:20:1.00:0.50:0.09: 0.15), (MEZLOCILLIN SODIUM:2:11286:2:20:1.00:0.50:0.09:0.15), (PHENACETIN:2:11286:3:19:1.00:0.40:0.10: 0.15), (PRETOMANID:2:11286:2:20:1.00:0.50:0.09:0.15), (AMINOLEVULINIC ACID:1:11287:3:19:1.00:0.25:0.05:0.08), (LEVAMISOLE:1:11287:3:19:1.00:0.25:0.05:0.08), (OTERACIL POTASSIUM:1:11287:3:19:1.00:0.25:0.05:0.08), (IPILIMUMAB:572:10715:0:23:1.00:1.00: 0.96:0.98), (GADOVERSETAMIDE:35:11252:16:7:1.00:0.69:0.83:0.75), (CREATINE MONOHYDRATE:17:11270:8:15:1.00:0.68:0.53:0.60), (ANTIHEMOPHILIC FACTOR, HUMAN RECOMBINANT RESIDUES 743-1636 DELETED:14:11273:10:13:1.00:0.58:0.52:0.55), (LEVOMEFOLIC ACIDMETHYLCOBALAMINTURMERIC:12:11275:15:8:1.00:0.44:0.60:0.51), (ATROPINE SULFATEDIFENOXIN HYDROCHLORIDE:7:11280:4:19:1.00:0.64:0.27:0.38), (ECHOTHIOPHATE IODIDE:7:11280:0:23:1.00:1.00:0.23:0.38), (BETAMETHASONE DIPROPIONATECLOTRIMAZOLEGENTAMICIN SULFATE:6:11281:8:15:1.00:0.43:0.29:0.34), (INSULIN DEGLUDECLIRAGLUTIDE:6:11281:3:20:1.00:0.67:0.23:0.34), (PARNAPARIN SODIUM:6:11281:2:21:1.00:0.75:0.22:0.34), (SOLCOSERYL:6:11281:2:21:1.00:0.75:0.22: 0.34), (ASPIRIN\CAFFEINEORPHENADRINE CITRATE:5:11282:3:20:1.00:0.63:0.20:0.30), (DACOMITINIB:5:11282:1:22:1.00:0.83:0.19: 0.30), (POLYETHYLENE GLYCOL 3350POLYETHYLENE GLYCOL 3350POTASSIUM CHLORIDE\SODIUM BICARBONATE\SODIUM CHLORIDE\SODIUM SULFATE ANHYDROUSPOTASSIUM CHLORIDE\SODIUM BICARBONATE\SODIUM CHLORIDE\SODIUM SULFATE ANHYDROUS:5:11282:0:23:1.00:1.00:0.18:0.30), (PROMESTRIENE:5:11282:2:21:1.00:0.7 1:0.19:0.30), (.ALPHA.-TOCOPHEROL.ALPHA.-TOCOPHEROL ACETATE.ALPHA.-TOCOPHEROL ACETATE, DL-ASCORBIC ACIDCHOLECALCIFEROLCOBALAMINCYANOCOBALAMINFLUORIDE IONFOLIC ACIDNIACINNIACINAMIDEPYRIDOXINEPYRIDOXINE HYDROCHLORIDE\RIBOFLAVIN\SODIUM ASCORBATE\SODIUM FLUORIDETHIAMINETHIAMINE MONONITRATEVITAMIN AVITA:4:11283:2:21:1.00:0.67:0.16:0.26), (BENPERIDOL:4:11283:6:17:1.00:0.40:0.19:0.26), (BINIMETINIB:4:11283:0:23:1.00:1.00:0.15:0.26), (CUSTIRSEN:4:11283:0:23:1.00:1.0 0:0.15:0.26), (DELAPRIL HYDROCHLORIDEINDAPAMIDE:4:11283:1:22:1.00:0.80:0.15:0.26), (DOCONEXENTIC OSAPENTPHOSPHATIDYL SERINE:4:11283:3:20:1.00:0.57:0.17:0.26), (LACTOFERRIN:4:11283:2:21:1.00:0.67:0.16: 0.26), (NIFLUMIC ACID:4:11283:0:23:1.00:1.00:0.15:0.26), (TIAGABINE:4:11283:4:19:1.00:0.50:0.17:0.26), (BELLADONNAERGOTAMINE TARTRATEPHENOBARBITAL:3:11284:4:19:1.00:0.43:0.14:0.21), (BICLOTYMOL:3:112 84:1:22:1.00:0.75:0.12:0.21), (BRIVUDINE:3:11284:1:22:1.00:0.75:0.12:0.21), (DIBUCAINE HYDROCHLORIDEPREDNISOLONE:3:11284:1:22:1.00:0.75:0.12:0.21), (EVOFOSFAMI DE:3:11284:4:19:1.00:0.43:0.14:0.21), (FUROSEMIDE SODIUMSPIRONOLACTONE:3:11284:3:20:1.00:0.50:0.13:0.21), (ISAVUCONAZONIUM: 3:11284:1:22:1.00:0.75:0.12:0.21), (POLYSORBATE 80:3:11284:1:22:1.00:0.75:0.12:0.21), (ACETAMINOPHENCHLORPHENIRAMINEDEXT ROMETHORPHANPHENYLEPHRINE:2:11285:1:22:1.00:0.67:0.08:0.15), (ACETAMINOP HENDIPHENHYDRAMINE HYDROCHLORIDEPHENYLEPHRINE HYDROCHLORIDE:2:11285:2:21:1.00:0.50:0.09:0.15), (CYCLOPHOSPHAMIDEDOXOR UBICINPREDNISONEVINCRISTINE:2:11285:2:21:1.00:0.50:0.09:0.15), (CYTARABINE HYDROCHLORIDE:2:11285:2:21:1.00:0.50:0.09:0.15), (DEXTROMETHORPHAN HYDROBROMIDEDOXYLAMINE SUCCINATE:2:11285:1:22:1.00:0.67:0.08:0.15), (LIDOCAINE HYDROCHLORIDEMETHYLPREDNISOLONE ACETATE:2:11285:2:21:1.00:0.50:0.09:0.15), (MALATHION:2:11285:2:21:1.00:0.50:0.09: 0.15), (NALIDIXIC ACID:2:11285:2:21:1.00:0.50:0.09:0.15), (BEZLOTOXUMAB:1:11286:3:20:1.00:0.25:0.05: 0.08), (CARGLUMIC ACID:1:11286:3:20:1.00:0.25:0.05:0.08), (COAGULATION FACTOR VII HUMAN:1:11286:3:20:1.00:0.25:0.05:0.08), (LOBAPLATIN:0:11287:3:20:1.00:0.00:0.00:#NUM!), (ATAZANAVIR SULFATE:279:11007:0:24:1.00:1.00:0.92:0.96), (THYMOCYTE IMMUNE GLOBULIN NOS:272:11014:0:24:1.00:1.00:0.92:0.96), (CALCIUM PHOSPHATE, DIBASIC, ANHYDROUSFERROUS SUCCINATEMAGNESIUM HYDROXIDE:26:11260:10:14:1.00:0.72:0.65:0.68), (BECLOMETHASONE DIPROPIONATEFORMOTEROL FUMARATE:19:11267:0:24:1.00:1.00:0.44:0.61), (CODEINE PHOSPHATEPHENYLEPHRINE HYDROCHLORIDEPROMETHAZINE HYDROCHLORIDE:13:11273:2:22:1.00:0.87:0.37:0.52), (BORIC ACIDSODIUM BORATE:11:11275:2:22:1.00:0.85:0.33:0.48), (SAFINAMIDE MESYLATE:10:11276:9:15:1.00:0.53:0.40:0.45), (SENNOSIDE A:9:11277:11:13:1.00:0.45:0.41:0.43), (VADASTUXIMAB TALIRINE:9:11277:6:18:1.00:0.60:0.33:0.43), (CHLORPROMAZINE HYDROCHLORIDEPHENOBARBITALPROMETHAZINE HYDROCHLORIDE:8:11278:1:23:1.00:0.89:0.26:0.40), (DIBUCAINE:7:11279:7:17:1.00:0.50:0.29:0.37), (PIRETANIDE:7:11279:5:19:1.00:0.58:0.27:0.37), (PORACTANT ALFA:7:11279:0:24:1.00:1.00:0.23:0.37), (AMPRENAVIR:6:11280:0:24:1.00:1.00:0.20:0.3 3), (EFALIZUMAB:6:11280:4:20:1.00:0.60:0.23:0.33), (ACETAMINOPHEN\CAFFEINEPR OPOXYPHENE:5:11281:7:17:1.00:0.42:0.23:0.29), (GLIMEPIRIDEPIOGLITAZONE HYDROCHLORIDE:5:11281:4:20:1.00:0.56:0.20:0.29), (HERBALSPLANTAGO OVATA LEAF:5:11281:1:23:1.00:0.83:0.18:0.29), (ORPHENADRINE HYDROCHLORIDE:5:11281:2:22:1.00:0.71:0.19:0.29), (PENBUTOLOL SULFATE:5:11281:7:17:1.00:0.42:0.23:0.29), (TOLPERISONE HYDROCHLORIDE:5:11281:4:20:1.00:0.56:0.20:0.29), (BIOTINCALCIUM PANTOTHENATECHOLINE BITARTRATECYANOCOBALAMINFOLIC ACIDINOSITOLNIACINAMIDEPYRIDOXINE\RIBOFLAVINTHIAMINE:4:11282:6:18:1.00:0.40:0.18:0.25), (DIHYDROERGOCRISTINE:4:11282:2:22:1.00:0.67:0.15:0.25), (DIPH THERIA AND TETANUS TOXOIDS AND ACELLULAR PERTUSSIS ADSORBED, INACTIVATED POLIOVIRUS AND HAEMOPHILUS B CONJUGATE AND HEPATITIS B VIRUS VACCINE:4:11282:0:24:1.00:1.00:0.14:0.25), (GLYCERINPROPYLENE GLYCOL:4:11282:1:23:1.00:0.80:0.15:0.25), (CODEINESISYMBRIUM OFFICINALE WHOLE:3:11283:1:23:1.00:0.75:0.12:0.20), (DAPSONEFERROUS OXALATE:3:11283:1:23:1.00:0.75:0.12:0.20), (DEUTETRABENAZINE:3:11283:12:12:1.00: 0.20:0.20:0.20), (ETILEFRINE:3:11283:2:22:1.00:0.60:0.12:0.20), (FERROUS ASPARTO GLYCINATEFERROUS FUMARATE:3:11283:3:21:1.00:0.50:0.13:0.20), (XANTOFYL PALMITATE:3:11283:1:23:1.00:0.75:0.12:0.20), (ACETAMINOPHENASCORBIC ACIDPHENIRAMINE MALEATE:2:11284:2:22:1.00:0.50:0.08:0.14), (CHLORHEXIDINE ACETATE:2:11284:2:22:1.00:0.50:0.08:0.14), (DALFOPRISTINQUINUPRISTIN:2:11284:3: 21:1.00:0.40:0.09:0.14), (GAMMA ORYZANOL:2:11284:0:24:1.00:1.00:0.08:0.14), (GLYCERINMEDIUM-CHAIN TRIGLYCERIDESPHOSPHOLIPIDSOYBEAN OIL:2:11284:2:22:1.00:0.50:0.08:0.14), (HOMOSALATEOCTINOXATEOCTOCRYLENE ZINC OXIDE:2:11284:1:23:1.00:0.67:0.08:0.14), (MIDOMAFETAMINE:2:11284:3:21:1.00:0.40: 0.09:0.14), (SODIUM SALICYLATE:2:11284:2:22:1.00:0.50:0.08:0.14), (TETANUS TOXOIDS:2:11284:1:23:1.00:0.67:0.08:0.14), (ACETAMINOPHENCHLORPHENIRAMINE MALEATEDEXTROMETHORPHAN HYDROBROMIDE:1:11285:4:20:1.00:0.20:0.05:0.08), (FERUMOXYTOL:1:11285:5:19:1.0 0:0.17:0.05:0.08), (TALIMOGENE LAHERPAREPVEC:40:11245:1:24:1.00:0.98:0.63:0.76), (EMTRICITABINERILPIVIRINE HYDROCHLORIDETENOFOVIR ALAFENAMIDE FUMARATE:12:11273:2:23:1.00:0.86:0.34:0.49), (BUTABARBITAL:10:11275:6:19:1.00:0.63:0.34:0.44), (TIROFIBAN:10:11275:1:24:1.00:0.91:0.29:0.44), (.ALPHA.-TOCOPHEROL ACETATE, DL-. BETA.-CAROTENEASCORBIC ACIDCALCIUM CARBONATECALCIUM PANTOTHENATECHOLECALCIFEROLCYANOCOBALAMINDOCUSATE SODIUMFERROUS FUMARATEFOLIC ACIDNIACINAMIDEPYRIDOXINE HYDROCHLORIDE\RIBOFLAVINTHIAMINE MONONITRATE\ZINC OXIDE:9:11276:1:24:1.00:0.90:0.27:0.42), (BEPRIDIL HYDROCHLORIDE:8:11277:10:15:1.00:0.44:0.35:0.39), (ABALOPARATIDE:7:11278:5:20: 1.00:0.58:0.26:0.36), (AMINOPYRINEANTIPYRINECAFFEINEPHENACETIN:7:11278: 3:22:1.00:0.70:0.24:0.36), (DEXTROMETHORPHAN HYDROBROMIDEGUAIFENESINPSEUDOEPHEDRINE HYDROCHLORIDE:7:11278:8:17:1.00:0.47:0.29:0.36), (CYSTINE:6:11279:6:19:1.00:0.50: 0.24:0.32), (NIFURTOINOL:6:11279:8:17:1.00:0.43:0.26:0.32), (CALCIUM SULFATEHERBALS:5:11280:3:22:1.00:0.63:0.19:0.29), (CHLORPROTHIXENE HYDROCHLORIDE:5:11280:0:25:1.00:1.00:0.17:0.29), (COLISTIN SULFATEHYDROCORTISONE ACETATENEOMYCIN SULFATETHONZONIUM BROMIDE:5:11280:1:24:1.00:0.83:0.17:0.29), (LUTEINIZING HORMONE:5:11280:2:23:1.00:0.71:0.18:0.29), (MAGNESIUM ACETATE:5:11280:5:20:1.00:0.50:0.20:0.29), (TECELEUKIN:5:11280:17:8:1.00:0.23:0.38: 0.29), (ACETAMINOPHEN\ASPIRINCODEINE PHOSPHATE:4:11281:1:24:1.00:0.80:0.14:0.24), (DIPHENOXYLATE HYDROCHLORIDE:4:11281:1:24:1.00:0.80:0.14:0.24), (ELM:4:11281:5:20:1.00:0.44:0.17: 0.24), (HYDROCORTISONEOXYTETRACYCLINE HYDROCHLORIDEPOLYMYXIN B SULFATE:4:11281:0:25:1.00:1.00:0.14:0.24), (PENTOXYVERINE CITRATE:4:11281:6:19:1.00:0.40:0.17:0.24), (TRIENTINE HYDROCHLORIDE:4:11281:9:16:1.00:0.31:0.20:0.24), (VINTAFOLIDE:4:11281:2:23:1.00: 0.67:0.15:0.24), (ACETAMINOPHENBUTABARBITALCAFFEINE:3:11282:3:22:1.00:0.5 0:0.12:0.19), (ACETAMINOPHEN\CAFFEINESALICYLIC ACID:3:11282:0:25:1.00:1.00:0.11:0.19), (BLOOD, BOVINE:3:11282:1:24:1.00:0.75:0.11:0.19), (CARBINOXAMINE:3:11282:1:24:1.00:0.75:0 0.11:0.19), (INDOBUFEN:3:11282:2:23:1.00:0.60:0.12:0.19), (MEDIUM-CHAIN TRIGLYCERIDES:3:11282:1:24:1.00:0.75:0.11:0.19), (NORGESTIMATE:3:11282:2:23:1.00: 0.60:0.12:0.19), (PASCOPYRUM SMITHII POLLEN:3:11282:10:15:1.00:0.23:0.17:0.19), (SULTHIAME:3:11282:6:19:1.00:0.33:0.14:0 0.19), (ULIPRISTAL:3:11282:5:20:1.00:0.38:0.13:0.19), (ACETAMINOPHENBUTYLSCOP OLAMINE BROMIDE:2:11283:10:15:1.00:0.17:0.12:0.14), (ASPIRIN\CAFFEINEPROMETHAZINES ALICYLAMIDE:2:11283:2:23:1.00:0.50:0.08:0.14), (BARIUM:2:11283:2:23:1.00:0.50:0.08: 0.14), (CEFTIZOXIME:2:11283:2:23:1.00:0.50:0.08:0.14), (CHLORPHENIRAMINE MALEATEIBUPROFENPSEUDOEPHEDRINE HYDROCHLORIDE:2:11283:5:20:1.00:0.29:0.09:0.14), (CILASTATIN SODIUM:2:11283:3:22:1.00:0.40:0.08:0.14), (CLARITHROMYCIN LACTOBIONATE:2:11283:3:22:1.00:0.40:0.08:0.14), (FACTOR IX COMPLEX:2:11283:2:23:1.00:0.50:0.08:0.14), (IFOSFAMIDEMESNA:2:11283:3:22:1.00:0 0.40:0.08:0.14), (ISOPROTERENOL:2:11283:2:23:1.00:0.50:0.08:0.14), (SILTUXIMAB:2:11 283:9:16:1.00:0.18:0.11:0.14), (HUMAN COLOSTRUM:1:11284:15:10:1.00:0.06:0.09:0.07), (ISONIAZIDPYRIDOXINE:1:11284:4:2 1:1.00:0.20:0.05:0.07), (BETA GLUCAN:12:11272:9:17:1.00:0.57:0.41:0.48), (THIAMYLAL:11:11273:11:15:1.00:0.50:0.4 2:0.46), (DIASTASE:10:11274:7:19:1.00:0.59:0.34:0.43), (AMLODIPINE BESYLATEATORVASTATIN CALCIUM TRIHYDRATE:9:11275:5:21:1.00:0.64:0.30:0.41), (THIOTHIXENE HYDROCHLORIDE:8:11276:9:17:1.00:0.47:0.32:0.38), (METAPROTERENOL SULFATE:7:11277:4:22:1.00:0.64:0.24:0.35), (NADH:7:11277:8:18:1.00:0.47:0.28:0.35), (PHENDIMETRAZINE:7:11277:3:23:1.00:0.70:0.23:0.35), (ALVERINE CITRATE:6:11278:2:24:1.00:0.75:0.20:0.32), (CHLORPHENIRAMINE MALEATEPSEUDOEPHEDRINE HYDROCHLORIDE:6:11278:2:24:1.00:0.75:0.20:0.32), (METHSUXIMIDE:6:11278:16:10:1 0.00:0.27:0.38:0.32), (METRONIDAZOLE BENZOATE:6:11278:5:21:1.00:0.55:0.22:0.32), (TOLMETIN SODIUM:6:11278:15:11:1.00:0.29:0.35:0.32), (LEVORPHANOL TARTRATE:5:11279:9:17:1.00:0.36:0.23:0.28), (LOFEPRAMINE:5:11279:5:21:1.00:0.50:0 0.19:0.28), (ZINC PICOLINATE:5:11279:4:22:1.00:0.56:0.19:0.28), (BELINOSTAT:4:11280:1:25:1.00:0.80:0.14:0.24), (BELLADONNA ALKALOIDS:4:11280:0:26:1.00:1.00:0.13:0.24), (CHOLECALCIFEROLCYANOCOBALA MIN CO-57\FOLIC ACIDPYRIDOXINE HYDROCHLORIDE:4:11280:2:24:1.00:0.67:0.14:0.24), (DIFLUCORTOLONE:4:11280:0:26: 1.00:1.00:0.13:0.24), (EMU OIL:4:11280:2:24:1.00:0.67:0.14:0.24), (PEMOLINE:4:11280:3:23:1.00:0.57:0.15:0.24), (T RIFLUSAL:4:11280:1:25:1.00:0.80:0.14:0.24), (ACETAMINOPHENCHLORPHENIRAMINE MALEATEDEXTROMETHORPHAN HYDROBROMIDEPHENYLEPHRINE HYDROCHLORIDE:3:11281:0:26:1.00:1.00:0.10:0.19), (ATENOLOL HYDROCHLORIDE:3:11281:1:25:1.00:0.75:0.11:0.19), (CHOLIC ACID:3:11281:6:20:1.00:0.33:0.13:0.19), (ETOMIDATE SULFATE:3:11281:0:26:1.00:1.00:0.10:0.19), (FLURAZEPAM MONOHYDROCHLORIDE:3:11281:1:25:1.00:0.75:0.11:0.19), (HEXACHLOROPHENE:3:1 1281:4:22:1.00:0.43:0.12:0.19), (NANDROLONE:3:11281:2:24:1.00:0.60:0.11:0.19), (SOB UZOXANE:3:11281:6:20:1.00:0.33:0.13:0.19), (STRONTIUM CHLORIDE:3:11281:5:21:1.00:0.38:0.13:0.19), (UNDECYLENIC ACID:3:11281:11:15:1.00:0.21:0.17:0.19), (ASCORBIC ACIDNIACINAMIDEPYRIDOXINE HYDROCHLORIDE\RIBOFLAVINTHIAMINE HYDROCHLORIDE:2:11282:2:24:1.00:0.50:0.08:0.13), (FENSPIRIDE HYDROCHLORIDE:2:11282:2:24:1.00:0.50:0.08:0.13), (ACETONE:1:11283:3:23:1.00:0.25: 0.04:0.07), (IOXILAN:1:11283:0:26:1.00:1.00:0.04:0.07), (PROPYPHENAZONE:1:11283:3: 23:1.00:0.25:0.04:0.07), (RABIES VACCINE:1:11283:1:25:1.00:0.50:0.04:0.07), (TETRACAINETETRACAINE HYDROCHLORIDE:22:11261:7:20:1.00:0.76:0.52:0.62), (SAQUINAVIR MESYLATE:21:11262:0:27:1.00:1.00:0.44:0.61), (CALCIUM CARBIMIDE:11:11272:9:18:1.00:0.55:0.38:0.45), (GANIRELIX ACETATE:11:11272:0:27:1.00:1.00:0.29:0.45), (POTASSIUM NITRATE\SODIUM FLUORIDE:10:11273:2:25:1.00:0.83:0.29:0.43), (ORGOTEIN:9:11274:6:21:1.00:0.60:0.30: 0.40), (RISEDRONATE SODIUM HEMI-PENTAHYDRATE:9:11274:1:26:1.00:0.90:0.26:0.40), (COBICISTAT:8:11275:1:26:1.00:0.8 9:0.24:0.37), (MIRIPLATIN:8:11275:12:15:1.00:0.40:0.35:0.37), (MINERALS\OMEGA-3 FATTY ACIDSVITAMINS:7:11276:2:25:1.00:0.78:0.22:0.34), (PANIPENEM:7:11276:3:24:1.00:0.7 0:0.23:0.34), (ACETAMINOPHENBELLADONNA LEAFCAFFEINEOPIUM TINCTURE:6:11277:1:26:1.00:0.86:0.19:0.31), (SODIUM FLUORIDEVITAMINS:6:11277:16:11:1.00:0.27:0.35:0.31), (HEPTAMINOL:5:11278:5:22: 1.00:0.50:0.19:0.27), (HYALURONIDASE RECOMBINANT HUMAN:5:11278:2:25:1.00:0.71:0.17:0.27), (CEFOTETAN DISODIUM:4:11279:1:26:1.00:0.80:0.13:0.23), (CIPROFLOXACINDEXAMETHASONE:4: 11279:18:9:1.00:0.18:0.31:0.23), (FLUPREDNIDENE ACETATEMICONAZOLE NITRATE:4:11279:5:22:1.00:0.44:0.15:0.23), (HYALURONIDASE:4:11279:1:26:1.00:0.80: 0.13:0.23), (RURIOCTOCOG ALFA PEGOL:4:11279:0:27:1.00:1.00:0.13:0.23), (SODIUM GLYCEROPHOSPHATE ANHYDROUS:4:11279:0:27:1.00:1.00:0.13:0.23), (ACETAMINOPHENAMOBARBITAL CAFFEINECODEINE PHOSPHATE:3:11280:2:25:1.00:0.60:0.11:0.18), (BUFLOMEDIL:3:11280:2:25:1.00:0.60:0 0.11:0.18), (CHLOROXYLENOL:3:11280:1:26:1.00:0.75:0.10:0.18), (CYSTEAMINE:3:1128 0:2:25:1.00:0.60:0.11:0.18), (FENOFIBRATEPRAVASTATIN:3:11280:1:26:1.00:0.75:0.10: 0.18), (PHOSPHORIC ACID:3:11280:1:26:1.00:0.75:0.10:0.18), (AMOROLFINE HYDROCHLORIDE:2:11281:2:25:1.00:0.50:0.07:0.13), (ANTITHROMBIN III HUMANCOAGULATION FACTOR IX HUMANCOAGULATION FACTOR VII HUMANCOAGULATION FACTOR X HUMANPROTEIN CPROTEIN S HUMANPROTHROMBIN:2:11281:0:27:1.00:1.00:0.07:0.13), (ARTICAINE:2:11281:1:26:1 0.00:0.67:0.07:0.13), (DUVELISIB:2:11281:2:25:1.00:0.50:0.07:0.13), (ETHYLENE GLYCOL:2:11281:2:25:1.00:0.50:0.07:0.13), (IODINEIOFLUPANE I-123:2:11281:0:27:1.00:1.00:0.07:0.13), (LINOLEIC ACID:2:11281:4:23:1.00:0.33:0.08:0.13), (OCTENIDINEPHENOXYETHANOL:2:11281:2: 25:1.00:0.50:0.07:0.13), (POLYMYXIN B SULFATETRIMETHOPRIM:2:11281:3:24:1.00:0.40:0.08:0.13), (RILONACEPT:2:11281:4: 23:1.00:0.33:0.08:0.13), (URACIL:2:11281:14:13:1.00:0.13:0.13:0.13), (4-HYDROXYBUTANOIC ACID:1:11282:4:23:1.00:0.20:0.04:0.07), (ANTIHEMOPHILIC FACTOR PORCINE, B-DOMAIN TRUNCATED RECOMBINANT:1:11282:1:26:1.00:0.50:0.04:0.07), (BOTULINUM TOXIN NOS:1:11282:4:23:1.00:0.20:0.04:0.07), (BISACODYLGLYCERINMINERAL OILSODIUM PHOSPHATE, DIBASIC\SODIUM PHOSPHATE, MONOBASIC:38:11244:12:16:1.00:0.76:0.70:0.73), (ZUCLOPENTHIXOL DECANOATE:25:11257:18:10:1.00:0.58:0.71:0.64), (DOXYLAMINEPYRIDOXINE:12:112 70:3:25:1.00:0.80:0.32:0.46), (GLATIRAMER:12:11270:9:19:1.00:0.57:0.39:0.46), (CARBI DOPAMELEVODOPA:11:11271:6:22:1.00:0.65:0.33:0.44), (HOMATROPINE HYDROBROMIDE:11:11271:4:24:1.00:0.73:0.31:0.44), (RED CLOVER:10:11272:13:15:1.00:0.43:0.40:0.42), (DASABUVIR SODIUM MONOHYDRATEOMBITASVIR HEMINONAHYDRATEPARITAPREVIR DIHYDRATERITONAVIR:9:11273:5:23:1.00:0.64:0.28:0.39), (ACETAMINOPHENCHLO RPHENIRAMINE MALEATEPHENYLEPHRINE HYDROCHLORIDE OR CETIRIZINE HYDROCHLORIDE OR CHLORPHENIRAMINE MALEATE OR CLEMASTINE FUMARATE OR DIPHENHYDRAMINE HYDROCHLORIDE OR FEXOFENADINE HYDROCHLORIDE OR LORATADINE:8:11274:5:23:1.00:0.62:0.26:0.36), (BENZOYL PEROXIDEERYTHROMYCIN:8:11274:4:24:1.00:0.67:0.25:0.36), (CLONIXINCYCLOBE NZAPRINE:8:11274:9:19:1.00:0.47:0.30:0.36), (CYCLOPHOSPHAMIDEEPIRUBICIN HYDROCHLORIDEFLUOROURACIL:8:11274:6:22:1.00:0.57:0.27:0.36), (COLI:8:11274:10:18:1.00:0.44:0.31:0.36), (IRBESARTANTRICHLORMETHIAZIDE:8:112 74:9:19:1.00:0.47:0.30:0.36), (SERTINDOLE:8:11274:19:9:1.00:0.30:0.47:0.36), (CALCIUM GLYCEROPHOSPHATECALCIUM PHOSPHATEGLUTAMIC ACIDVITAMIN B:7:11275:0:28:1.00:1.00:0.20:0.33), (CARBOXYMETHYLCELLULOSE SODIUMGELATINPECTIN:6:11276:6:22:1.00:0.50:0.21:0.30), (DEOXYRIBONUCLIEC ACID:6:11276:20:8:1.00:0.23:0.43:0.30), (HARPAGOPHYTUM PROCUMBENS ROOT:6:11276:5:23:1.00:0.55:0.21:0.30), (TRAPIDIL:6:11276:5:23:1.00:0.55:0.21:0.30), ( ALUMINUM ACETATE:5:11277:2:26:1.00:0.71:0.16:0.26), (ATOVAQUONEPROGUANIL:5:11277:3:25: 1.00:0.63:0.17:0.26), (GINKGO BILOBA LEAF EXTRACT:5:11277:3:25:1.00:0.63:0.17:0.26), (INFLUENZA A VIRUS A/BRISBANE/10/2010 (H1N1) ANTIGEN (MDCK CELL DERIVED, PROPIOLACTONE INACTIVATED)\INFLUENZA A VIRUS A/VICTORIA/361/2011 IVR-165 (H3N2) ANTIGEN (MDCK CELL DERIVED, PROPIOLACTONE INACTIVATED)\INFLUENZA B VIRUS B/WISCONSIN/1/2010) ANTIGEN (MDCK CELL DERIVED, PROPIOLACTONE INACTIVATED):5:11277:3:25:1.00:0.63:0.17:0.26), (ALGINIC ACIDALUMINUM HYDROXIDEMAGNESIUM CARBONATE:4:11278:3:25:1.00:0.57:0.14:0.22), (ASPARTAME:4:11278:5:23:1.00:0.44:0 0.15:0.22), (FERROUS HYDROXIDE:4:11278:0:28:1.00:1.00:0.13:0.22), (RETAPAMULIN:4:11278:1:27:1.00:0.80: 0.13:0.22), (PERTUSSIS VACCINE:3:11279:0:28:1.00:1.00:0.10:0.18), (ACEPROMAZINE MALEATEACEPROMETAZINECLORAZEPATE DIPOTASSIUM:2:11280:3:25:1.00:0.40:0.07:0.13), (BROMPHENIRAMINEPSEUDOEPHE DRINE:2:11280:2:26:1.00:0.50:0.07:0.13), (DIMETHICONEPETROLATUM\ZINC OXIDE:2:11280:0:28:1.00:1.00:0.07:0.13), (MAGNESIUM PHOSPHATE, TRIBASIC, PENTAHYDRATE:2:11280:2:26:1.00:0.50:0.07:0.13), (OXYMETHOLONE:2:11280:3:25:1.00:0.40:0.07:0.13), (OXYTETRACYCLINEOXYTETRACYCLINE HYDROCHLORIDE:2:11280:0:28:1.00:1.00:0.07:0.13), (PYRILAMINE:2:11280:2:26:1.00: 0.50:0.07:0.13), (STREPTOZOCIN:2:11280:4:24:1.00:0.33:0.08:0.13), (ASPIRINCHLORP HENIRAMINE MALEATEPHENYLEPHRINE BITARTRATE:1:11281:4:24:1.00:0.20:0.04:0.07), (CLOBETASONE BUTYRATENYSTATINOXYTETRACYCLINE CALCIUM:1:11281:8:20:1.00:0.11:0.05:0.07), (VALERIAN EXTRACT:1:11281:5:23:1.00:0.17:0.04:0.07), (TOLCAPONE:18:11263:9:20:1.00:0.67:0.47: 0.55), (FOLLICLE STIMULATING HORMONE BETA POLYPEPTIDELUTROPIN ALFA:14:11267:5:24:1.00:0.74:0.37:0.49), (OZAGREL:13:11268:7:22:1.00:0.65:0.37:0.47), (SAFFLOWER OIL:13:11268:3:26:1.00:0.81:0.33:0.47), (GOLD SODIUM THIOMALATE:11:11270:5:24:1.00:0.69:0.31:0.43), (CHOLECALCIFEROLGENISTEINZINC GLYCINATE:9:11272:16:13:1.00:0.36:0.41:0.38), (HERBALSHOMEOPATHICSGOLDEN SEAL:9:11272:15:14:1.00:0.38:0.39:0.38), (PYRIMETHAMINESULFADOXINE:7:11274:4: 25:1.00:0.64:0.22:0.33), (ABEMACICLIB:6:11275:1:28:1.00:0.86:0.18:0.29), (AMRUBICIN HYDROCHLORIDE:6:11275:1:28:1.00:0.86:0.18:0.29), (HYDROCORTISONEMICONAZ OLE:6:11275:4:25:1.00:0.60:0.19:0.29), (IRONVITAMIN B:6:11275:2:27:1.00:0.75:0.18:0.29), (PLASMA PROTEIN FRACTION (HUMAN):6:11275:13:16:1.00:0.32:0.27:0.29), (PREDNISOLONE SODIUM METAZOATE:6:11275:15:14:1.00:0.29:0.30:0.29), (RASPBERRY KETONE:6:11275:7:22:1.00:0.46:0.21:0.29), (TALTIRELIN:6:11275:4:25:1.00:0.60:0.19:0.29), (ALMOTRIPTAN:5:11276:2:27:1.00:0.71:0.16:0.26), (ASCORBIC ACIDVITAMIN B COMPLEX:5:11276:9:20:1.00:0.36:0.20:0.26), (TROPATEPINE:5:11276:4:25:1.00:0.56:0.1 7:0.26), (CAPSICUM OLEORESIN:4:11277:3:26:1.00:0.57:0.13:0.22), (DOVITINIB LACTATE:4:11277:0:29:1.00:1.00:0.12:0.22), (ETELCALCETIDE HYDROCHLORIDE:4:11277:3:26:1.00:0.57:0.13:0.22), (LESINURAD:4:11277:8:21:1.00:0.33:0.16:0.22), (MINERALSPROBIOTICS NOSVITAMINS:4:11277:11:18:1.00:0.27:0.18:0.22), (NAFTIFINE:4:11277:2:27:1.00:0.67: 0.13:0.22), (TRICAPRYLIN:4:11277:3:26:1.00:0.57:0.13:0.22), (CANRENOATE POTASSIUMTROMETHAMINE:3:11278:1:28:1.00:0.75:0.10:0.17), (HERBSUNSPECIFIE D INGREDIENT:3:11278:5:24:1.00:0.38:0.11:0.17), (METHYL ALCOHOL:3:11278:2:27:1.00:0.60:0.10:0.17), (NOSCAPINE:3:11278:1:28:1.00:0.75:0.10: 0.17), (PIPOBROMAN:3:11278:3:26:1.00:0.50:0.10:0.17), (POTASSIUM SORBATE:3:11278:4:25:1.00:0.43:0.11:0.17), (TANEZUMAB:3:11278:2:27:1.00:0.60:0.10: 0.17), (TECHNETIUM TC-99M MEDRONATE:3:11278:0:29:1.00:1.00:0.09:0.17), (ACLARUBICIN:2:11279:2:27:1.00:0.50: 0.07:0.12), (EFLORNITHINE:2:11279:2:27:1.00:0.50:0.07:0.12), (QUINIDINE SULFATE:2:11279:2:27:1.00:0.50:0.07:0.12), (TECHNETIUM TC-99M TETROFOSMIN:2:11279:1:28:1.00:0.67:0.07:0.12), (FLOXURIDINE:1:11280:3:26:1.00:0.2: 0.04:0.06), (PENICILLIN G PROCAINE:1:11280:3:26:1.00:0.25:0.04:0.06), (CORTICOSTEROID NOS:700:10580:0:30:1.00:1.00:0.96:0.98), (ENFUVIRTIDE:63:11217:11:19:1.00:0.85:0.77: 0.81), (PANCURONIUM:15:11265:10:20:1.00:0.60:0.43:0.50), (PAPAVERINE HYDROCHLORIDE:14:11266:2:28:1.00:0.88:0.33:0.48), (NAFARELIN ACETATE:12:11268:5:25:1.00:0.71:0.32:0.44), (PROPANTHELINE BROMIDE:11:11269:7:23:1.00:0.61:0.32:0.42), (IPRAGLIFLOZIN:10:11270:15:15:1.00:0.4 0:0.40:0.40), (AMINO ACIDSVITAMINS:8:11272:9:21:1.00:0.47:0.28:0.35), (CALCIUM PANTOTHENATECUPRIC SULFATECYANOCOBALAMINFERROUS ASPARTO GLYCINATEFERROUS FUMARATEFOLIC ACIDMAGNESIUM SULFATENIACINAMIDEPYRIDOXINE HYDROCHLORIDE\RIBOFLAVIN\SODIUM ASCORBATETHIAMINE MONONITRATE\ZINC SULFATE:7:11273:8:22:1.00:0.47:0.24:0.32), (FLUTOPRAZEPAM:7:11273:2:28:1.00:0.78: 0.20:0.32), (SULBUTIAMINE:6:11274:10:20:1.00:0.38:0.23:0.29), (AMINO ACIDSSOYBEAN OILVITAMINS:5:11275:0:30:1.00:1.00:0.14:0.25), (DOXYCYCLINE CALCIUM:5:11275:1:29:1.00:0.83:0.15:0.25), (GLECAPREVIRPIBRENTASVIR:5:11275:1: 29:1.00:0.83:0.15:0.25), (GOLDENSEAL:5:11275:3:27:1.00:0.63:0.16:0.25), (GUSELKU MAB:5:11275:1:29:1.00:0.83:0.15:0.25), (MEASLES-MUMPS-RUBELLA VIRUS VACCINE:5:11275:0:30:1.00:1.00:0.14:0.25), (ACETAMINOPHENDEXTROMETHORPH ANPSEUDOEPHEDRINE:4:11276:0:30:1.00:1.00:0.12:0.21), (ALFALFAALFALFA WHOLE:4:11276:3:27:1.00:0.57:0.13:0.21), (GELATINTHROMBIN HUMAN:4:11276:0:30:1.00:1.00:0.12:0.21), (OXYQUINOLINE SULFATE\SODIUM LAURYL SULFATE:4:11276:10:20:1.00:0.29:0.17:0.21), (RANOLAZINE HYDROCHLORIDE:4:11276:1:29:1.00:0.80:0.12:0.21), (RESERPINE:4:11276:4:26:1.00:0.50:0.13:0.21), (ALACEPRIL:3:11277:1:29:1.00:0.75:0.09:0.17), (BENOXINATE:3:11277:0: 30:1.00:1.00:0.09:0.17), (CALCIUM LACTATECALCIUM PHOSPHATECHOLECALCIFEROL:3:11277:3:27:1.00:0.50:0.10:0.17), (FLURBIPROFEN SODIUM:3:11277:0:30:1.00:1.00:0.09:0.17), (POLOXAMER:3:11277:3:27:1.00:0.50:0.10: 0.17), (ACETAMINOPHEN OR ACETAMINOPHEN\ASPIRIN\CAFFEINE OR ASPIRIN\CAFFEINE:2:11278:2:28:1.00:0.50:0.07:0.12), (ALGINIC ACIDALUMINUM HYDROXIDEMAGNESIUM TRISILICATE:2:11278:2:28:1.00:0.50:0.07:0.12), (ALPHA-TOCOPHEROLCHOLECALCIFEROL:2:11278:5:25:1.00:0.29:0.07:0.12), (IOXAGLATE MEGLUMINEIOXAGLATE SODIUM:1:11279:3:27:1.00:0.25:0.04:0.06), (METHYLPREDNISOLONE OR METHYLPREDNISOLONE ACETATE:1:11279:20:10:1.00:0.05:0.09:0.06), (TEMOCILLIN:1:11279:3:27:1.00:0.25:0.0 4:0.06), (LOPINAVIRRITONAVIR:380:10899:0:31:1.00:1.00:0.92:0.96), (CHLOROQUINE CHLOROQUINE PHOSPHATE:83:11196:2:29:1.00:0.98:0.74:0.84), (CLOMIPHENE:72:11207:0:31:1.00:1.00: 0.70:0.82), (NELFINAVIR:19:11260:5:26:1.00:0.79:0.42:0.55), (HERBALSKARAYA GUM:17:11262:7:24:1.00:0.71:0.41:0.52), (CARBOXYMETHYLCELLULOSE SODIUMSORBINICATE:11:11268:4:27:1.00:0.73:0.29:0.42), (CLOTRIMAZOLEHYDRO CORTISONE:11:11268:2:29:1.00:0.85:0.28:0.42), (MITIGLINIDE CALCIUM HYDRATE:11:11268:1:30:1.00:0.92:0.27:0.42), (ARCTOSTAPHYLOS UVA-URSI LEAFDIOICA:9:11270:16:15:1.00:0.36:0.38:0.37), (AVOBENZONEHOMOSALATEOCTISALA TE:9:11270:18:13:1.00:0.33:0.41:0.37), (BACITRACINNEOMYCINPOLYMYXIN B:9:11270:6:25:1.00:0.60:0.26:0.37), (CHAMOMILE:9:11270:12:19:1.00:0.43:0.32:0.37), (CHLORMEZANONE:9:11270:6:25:1.00:0.60:0.26:0.37), (IXAZOMIB CITRATE:9:11270:8:23:1.00:0.53:0.28:0.37), (ASPIRINPRAVASTATIN:8:11271:1:30:1.00: 0.89:0.21:0.34), (TROGLITAZONE:8:11271:8:23:1.00:0.50:0.26:0.34), (ACETAMINOPHE NOPIUM:7:11272:2:29:1.00:0.78:0.19:0.31), (CAMOSTAT:7:11272:3:28:1.00:0.70:0.20:0.31), (DEXAMETHASONEFRAMYCETIN SULFATEGRAMICIDIN:7:11272:4:27:1.00:0.64:0.21:0.31), (ATENOLOLHYDROCHLOR OTHIAZIDE:6:11273:4:27:1.00:0.60:0.18:0.28), (CYANAMIDE:6:11273:19:12:1.00:0.24:0.33:0.28), (PARSLEY:6:11273:21:10:1.00:0.22:0.38:0.28), (GINKGOHEPTAMINOLTROX ERUTIN:5:11274:0:31:1.00:1.00:0.14:0.24), (KERATINSENNA LEAF:5:11274:1:30:1.00:0.83:0.14:0.24), (RECUTITA:5:11274:14:17:1.00:0.26:0.23:0.24), (MECLOFENAMATE SODIUM:5:11274:3:28:1.00:0.63:0.15:0.24), (SAGE:5:11274:1:30:1.00:0.83:0.14:0.24), (A CETAMINOPHEN\CAFFEINECODEINE PHOSPHATEMEPROBAMATE:4:11275:0:31:1.00:1.00:0.11:0.21), (ACETAMINOPHENP HENYLEPHRINE:4:11275:4:27:1.00:0.50:0.13:0.21), (AMLODIPINEHYDROCHLOROTH IAZIDEVALSARTAN:4:11275:0:31:1.00:1.00:0.11:0.21), (MONTANA:4:11275:8:23:1.00:0.33:0.15:0.21), (CEFAMANDOLE:4:11275:3:28:1.00:0.57: 0.13:0.21), (DIETHYLAMINE SALICYLATE:4:11275:1:30:1.00:0.80:0.12:0.21), (EMOLLIENTS:4:11275:0:31:1.00:1.00: 0.11:0.21), (LAMBROLIZUMAB:4:11275:0:31:1.00:1.00:0.11:0.21), (METAMIZOLEPRO POXYPHENE:4:11275:0:31:1.00:1.00:0.11:0.21), (NANDROLONE DECANOATE:4:11275:1:30:1.00:0.80:0.12:0.21), (ALUMINUM HYDROXIDEALUMINUM PHOSPHATEGUAR GUM\MAGNESIUM HYDROXIDE:3:11276:1:30:1.00:0.75:0.09:0.16), (CALCIUM CARBONATEMAGNESIUM CARBONATE:3:11276:2:29:1.00:0.60:0.09:0.16), (CRYOPRECIPITATED ANTIHEMOPHILIC FACTOR (HUMAN):3:11276:1:30:1.00:0.75:0.09:0.16), (MICONAZOLE NITRATEPETROLATUM\ZINC OXIDE:3:11276:2:29:1.00:0.60:0.09:0.16), (PHENOXYMETHYLPENICILLIN CALCIUM:3:11276:3:28:1.00:0.50:0.10:0.16), (ACETAMINOPHENDEXTROMETHORPH AN HYDROBROMIDEDOXYLAMINE SUCCINATEPHENYLEPHRINE:2:11277:1:30:1.00:0.67:0.06:0.11), (AMLODIPINE MESYLATE:2:11277:2:29:1.00:0.50:0.06:0.11), (FLUOROMETHOLONE ACETATE:2:11277:2:29:1.00:0.50:0.06:0.11), (PLATINUM:2:11277:3:28:1.00:0.40:0.07:0.11), (SECOBARBITAL:2:11277:2:29:1.00:0.50:0.06:0.11), (BELIMUMAB:283:10995:1:31:1 0.00:1.00:0.90:0.95), (DOTHIEPINDOTHIEPIN HYDROCHLORIDE:12:11266:3:29:1.00:0.80:0.29:0.43), (GLYCEROL PHENYLBUTYRATE:11:11267:4:28:1.00:0.73:0.28:0.41), (THEANINE:10:11268:4:28:1.00: 0.71:0.26:0.38), (CALCIUM CHLORIDE\POTASSIUM CHLORIDE\SODIUM CHLORIDE\SODIUM LACTATESORBITOL:9:11269:5:27:1.00:0.64:0.25:0.36), (ESTRAMUSTINE:8:11270:3:29: 1.00:0.73:0.22:0.33), (BROMFENACBROMFENAC SODIUM:7:11271:4:28:1.00:0.64:0.20:0.30), (CAFFEINEPHENOBARBITAL:7:11271:4:28: 1.00:0.64:0.20:0.30), (PHENAZEPAM:7:11271:3:29:1.00:0.70:0.19:0.30), (QUININE BISULFATE:7:11271:3:29:1.00:0.70:0.19:0.30), (ACETAMINOPHENALCOHOLPHENYL TOLOXAMINE CITRATE:6:11272:0:32:1.00:1.00:0.16:0.27), (OMARIGLIPTIN:6:11272:2:30:1.00:0.75:0.1 7:0.27), (PHENYLBUTAZONE:6:11272:7:25:1.00:0.46:0.19:0.27), (BIFIDUM:5:11273:2:30:1.00:0.71:0.14:0.24), (CHOLINETRAZODONE HYDROCHLORIDE:5:11273:19:13:1.00:0.21:0.28:0.24), (ERYTHROMYCIN STEARATE:5:11273:1:31:1.00:0.83:0.14:0.24), (FROVATRIPTAN:5:11273:3:29:1.00:0.63: 0.15:0.24), (MOMETASONE FUROATE MONOHYDRATE:5:11273:0:32:1.00:1.00:0.14:0.24), (REISHI:5:11273:4:28:1.00:0.56:0.15: 0.24), (AMYLASE:4:11274:3:29:1.00:0.57:0.12:0.20), (BECLOMETHASONE DIPROPIONATEBETAMETHASONE SODIUM PHOSPHATE:4:11274:1:31:1.00:0.80:0.11:0.20), (CIPROFLOXACIN LACTATE:4:11274:0:32:1.00:1.00:0.11:0.20), (DEXAMETHASONEOXYTETRACYCLIN E:4:11274:3:29:1.00:0.57:0.12:0.20), (HAEMOPHILUS INFLUENZAE TYPE B:4:11274:1:31:1.00:0.80:0.11:0.20), (INFLUENZA VIRUS VACCINE, SWINE:4:11274:1:31:1.00:0.80:0.11:0.20), (BUTAMIRATE CITRATE:3:11275:2:30:1.00:0.60:0.09:0.16), (CALAMINE LOTION:3:11275:1:31:1.00:0.75:0.09:0.16), (CINNAMON OIL:3:11275:6:26:1.00:0.33:0.10:0.16), (EUCALYPTUS OIL:3:11275:2:30:1.00:0.60:0.09:0.16), (KAOLIN:3:11275:4:28:1.00:0.43:0.10:0.16), (RUT IN:3:11275:2:30:1.00:0.60:0.09:0.16), (SODIUM GLYCEROPHOSPHATE:3:11275:1:31:1.00:0.75:0.09:0.16), (ARTICAINEEPINEPHRINE: 2:11276:2:30:1.00:0.50:0.06:0.11), (HEPATITIS B VIRUS SUBTYPE ADW2 HBSAG SURFACE PROTEIN ANTIGEN:2:11276:1:31:1.00:0.67:0.06:0.11), (RINGER'S SOLUTION, LACTATED:2:11276:1:31:1.00:0.67:0.06:0.11), (RHAMNUS PURSHIANA BARK EXTRACT:1:11277:2:30:1.00:0.33:0.03:0.06), (BIVALIRUDIN:79:11198:1:32:1.00:0.99:0.71:0.83), (PEROSPIRONE:17:11260:17:16:1.00:0.50:0.52:0.51), (CYANOCOBALAMINPY RIDOXINE HYDROCHLORIDE\RIBOFLAVIN\SODIUM PANTOTHENATETHIAMINE HYDROCHLORIDEVITAMIN B COMPLEX:10:11267:10:23:1.00:0.50:0.30:0.38), (SULFANILAMIDE:8:11269:3:30:1.00:0.7 3:0.21:0.33), (CORTIVAZOL:7:11270:1:32:1.00:0.88:0.18:0.30), (HUMAN PAPILLOMAVIRUS QUADRIVALENT (TYPES 6, 11, 16, AND 18) VACCINE, RECOMBINANT:7:11270:3:30:1.00:0.70:0.19:0.30), (CLOBETASONE:6:11271:1:32:1.00:0 0.86:0.16:0.27), (BENZALKONIUM CHLORIDEBENZYL ALCOHOLCHLORHEXIDINE:4:11273:0:33:1.00:1.00:0.11:0.20), (CYTARABINE OCFOSFATE:4:11273:10:23:1.00:0.29:0.15:0.20), (FLORBETAPIR F-18:4:11273:4:29:1.00:0.50:0.12:0.20), (ISONIAZIDPYRIDOXINE HYDROCHLORIDE:4:11273:1:32:1.00:0.80:0.11:0.20), (NEISSERIA MENINGITIDIS GROUP A CAPSULAR POLYSACCHARIDE DIPHTHERIA TOXOID CONJUGATE ANTIGEN\NEISSERIA MENINGITIDIS GROUP C CAPSULAR POLYSACCHARIDE DIPHTHERIA TOXOID CONJUGATE ANTIGEN\NEISSERIA MENINGITIDIS GROUP W-135 CAPSULAR POLYSACCHARIDE DIPHTHERIA TOXOID CONJUGATE ANTIGEN\MENINGITIDIS:4:11273:6:27:1.00:0.40:0.13:0.20), (ORNITHINE:4:11273:2:31:1.00:0.67:0 0.11:0.20), (OSTREA EDULIS SHELL:4:11273:11:22:1.00:0.27:0.15:0.20), (BALSAM PERUCASTOR OIL:3:11274:1:32:1.00:0.75:0.09:0.15), (PREDNISOLONE HEMISUCCINATE:3:11274:1:32:1.00:0.75:0.09:0.15), (PROTHIPENDYL:3:11274:1:32:1.0 0:0.75:0.09:0.15), (CODEINE HYDROCHLORIDE:2:11275:2:31:1.00:0.50:0.06:0.11), (ISOMETHEPTENE:2:11275:2:31:1 0.00:0.50:0.06:0.11), (OPRELVEKIN:2:11275:5:28:1.00:0.29:0.07:0.11), (RIBONUCLEIC ACID:2:11275:2:31:1.00:0.50:0.06:0.11), (GIMERACIL:1:11276:3:30:1.00:0.25:0.03:0.06), (PRAZIQUANTEL:1:11276:3:30:1.00:0.25:0.03:0.06), (BROMOCRIPTINE MESYLATE:187:11089:0:34:1.00:1.00:0.85:0.92), (BUTORPHANOL TARTRATE:80:11196:0:34:1.00:1.00:0.70:0.82), (HUMAN CYTOMEGALOVIRUS IMMUNE GLOBULIN:29:11247:0:34:1.00:1.00:0.46:0.63), (BRINCIDOFOVIR:24:11252:12:22:1.00:0 0.67:0.52:0.59), (TABALUMAB:14:11262:22:12:1.00:0.39:0.54:0.45), (TEGASEROD:13:112 63:11:23:1.00:0.54:0.36:0.43), (RESLIZUMAB:11:11265:12:22:1.00:0.48:0.33:0.39), (ALG ELDRATE:10:11266:4:30:1.00:0.71:0.25:0.37), (CALCIUMERGOCALCIFEROL:10:11266: 8:26:1.00:0.56:0.28:0.37), (REBOXETINE:10:11266:3:31:1.00:0.77:0.24:0.37), (CODEINE PHOSPHATEGUAIFENESINPSEUDOEPHEDRINE HYDROCHLORIDE:9:11267:10:24:1.00:0.47:0.27:0.35), (FOLIC ACIDIRON POLYMALTOSE:9:11267:3:31:1.00:0.75:0.23:0.35), (HYDROCHLOROTHIAZIDEPROPR ANOLOL HYDROCHLORIDE:9:11267:5:29:1.00:0.64:0.24:0.35), (TULOBUTEROL HYDROCHLORIDE:9:11267:12:22:1.00:0.43:0.29:0.35), (TRIAMCINOLONE HEXACETONIDE:6:11270:0:34:1.00:1.00:0.15:0.26), (ZINC CITRATE:6:11270:14:20:1.00:0.30:0.23:0.26), (ASCORBIC ACIDCHROMIUMSELENIUMVANADYL SULFATE:5:11271:2:32:1.00:0.71:0.14:0.23), (CUMIN:5:11271:5:29:1.00:0.50:0.15:0.23), (ESKETAMINE:4:11272:1:33:1.00:0.80:0.11:0.19), (TALAZOPARIB:4:11272:0:34:1.00:1.0 0:0.11:0.19), (TOLMETIN:4:11272:12:22:1.00:0.25:0.15:0.19), (TRIBENOSIDE:4:11272:3: 31:1.00:0.57:0.11:0.19), (COLD CREAM:3:11273:2:32:1.00:0.60:0.09:0.15), (FLUOROURACILIRINOTECANLEUCOVO RINOXALIPLATIN:3:11273:2:32:1.00:0.60:0.09:0.15), (GLUCARPIDASE:3:11273:1:33:1.00:0.75:0.08:0.15), (MINERAL OIL EMULSIONPETROLATUM:3:11273:1:33:1.00:0.75:0.08:0.15), (NAFCILLIN SODIUM:3:11273:1:33:1.00:0.75:0.08:0.15), (PHENCYCLIDINE:3:11273:2:32:1.00:0.60:0.09:0.15), (TECHNETIUM TC-99M SESTAMIBI:3:11273:0:34:1.00:1.00:0.08:0.15), (TENOFOVIR ALAFENAMIDE FUMARATE:3:11273:1:33:1.00:0.75:0.08:0.15), (TENOFOVIR DISOPROXIL:3:11273:2:32:1.00:0.60:0.09:0.15), (CEPHALEXIN HYDROCHLORIDE:2:11274:3:31:1.00:0.40:0.06:0.11), (TETANICLOSTRIDIUM TETANI TOXOID ANTIGEN (FORMALDEHYDE INACTIVATED)DIPHTHERIAE TOXOID ANTIGEN (FORMALDEHYDE INACTIVATED):2:11274:16:18:1.00:0.11:0.10:0.11), (DEXPANTHENOLXYLOMETAZO LINE HYDROCHLORIDE:2:11274:2:32:1.00:0.50:0.06:0.11), (PARITAPREVIR:2:11274:2:32:1.0 0:0.50:0.06:0.11), (REFAMETINIB:2:11274:2:32:1.00:0.50:0.06:0.11), (SERTACONAZOLE NITRATE:2:11274:6:28:1.00:0.25:0.07:0.11), (TREPROSTINIL:577:10698:0:35:1.00:1.00:0 0.94:0.97), (APOMORPHINE:18:11257:9:26:1.00:0.67:0.41:0.51), (BUCOLOME:17:11258:1 0:25:1.00:0.63:0.40:0.49), (FENOPROFEN CALCIUM:16:11259:15:20:1.00:0.52:0.44:0.48), (IMIPRAMINE PAMOATE:16:11259:16:19:1.00:0.50:0.46:0.48), (CHLORPROMAZINEPROMETHAZINE: 15:11260:16:19:1.00:0.48:0.44:0.46), (LITHIUM SULFATE:14:11261:10:25:1.00:0.58:0.36:0.44), (CEFPIROME:13:11262:5:30:1.00:0.72:0.3 0:0.43), (DOCONEXENT ICOSAPENT FISH OIL OMEGA-3 FATTY ACIDS:12:11263:5:30:1.00:0.71:0.29:0.41), (FERROGLYCINE SULFATE:9:11266:2:33:1.00:0.82:0.21:0.34), (CEPHALOSPORIN C:7:11268:6:29:1.00:0.54:0.19:0.29), (NILUTAMIDE:7:11268:5:30:1.00:0.58:0.19:0.29), (B ALSAM PERU OILCASTOR OILTRYPSIN:6:11269:8:27:1.00:0.43:0.18:0.26), (BENFOTIAMINECYANOCOBALAMI NPYRIDOXINE HYDROCHLORIDE:6:11269:0:35:1.00:1.00:0.15:0.26), (ESSENTIAL OILS:6:11269:7:28:1.00:0.46:0.18:0.26), (PLANTAGO OVATA SEED COAT:6:11269:2:33:1.00:0.75:0.15:0.26), (VITAMIN A PALMITATE:6:11269:21:14:1.00:0.22:0.30:0.26), (ETHINYL ESTRADIOLETHYNODIOL:5:11270:14:21:1.00:0.26:0.19:0.22), (GENISTEIN:5:11270:9:2 6:1.00:0.36:0.16:0.22), (PHLEUM PRATENSE POLLEN:5:11270:0:35:1.00:1.00:0.13:0.22), (ACETIC ACIDDESONIDE:4:11271:1:34:1.00:0.80:0.11:0.19), (CAPECITABINEOXALIPLATIN:4: 11271:8:27:1.00:0.33:0.13:0.19), (ELETRIPTAN:4:11271:2:33:1.00:0.67:0.11:0.19), (MET RONIDAZOLESPIRAMYCIN:4:11271:0:35:1.00:1.00:0.10:0.19), (MILTEFOSINE:4:11271: 2:33:1.00:0.67:0.11:0.19), (MIZOLASTINE:4:11271:1:34:1.00:0.80:0.11:0.19), (ROLAPIT ANT:4:11271:3:32:1.00:0.57:0.11:0.19), (POTASSIUM CITRATE\SODIUM CITRATE:3:11272:1:34:1.00:0.75:0.08:0.15), (STANOZOLOL:3:11272:0:35:1.00:1.00:0.08: 0.15), (ALOSETRON:2:11273:6:29:1.00:0.25:0.06:0.10), (MEASLES VIRUS STRAIN ENDERS' ATTENUATED EDMONSTON LIVE ANTIGENMUMPS VIRUS STRAIN B LEVEL JERYL LYNN LIVE ANTIGENRUBELLA VIRUS STRAIN WISTAR RA 27/3 LIVE ANTIGEN:1:11274:0:35:1.00:1.00:0.03:0.05), (NITROUS OXIDE:170:11104:0:36:1.00:1.00:0.83:0.90), (DEOXYCHOLIC ACID:41:11233:1:35:1.00:0.98:0.54:0.69), (DARUNAVIRRITONAVIR:36:11238:20:16:1.0 0:0.64:0.69:0.67), (ATAZANAVIRRITONAVIR:24:11250:14:22:1.00:0.63:0.52:0.57), (CA RBOXYMETHYLCELLULOSE SODIUMHYPROMELLOSES:20:11254:13:23:1.00:0.61:0.47:0.53), (OTILONIUM BROMIDE:15:11259:3:33:1.00:0.83:0.31:0.45), (ALUMINUM HYDROXIDEDIMETHICONEDIPHENHYDRAMINEHYDROXIDE IONLIDOCAINE:11:11263:6:30:1.00:0.65:0.27:0.38), (DIETHYLPROPION:10:11264:3:33: 1.00:0.77:0.23:0.36), (.ALPHA.-LIPOIC ACID, (+/−)-:9:11265:2:34:1.00:0.82:0.21:0.33), (ACETAMINOPHENPHENYLEPHRINE HYDROCHLORIDE:9:11265:7:29:1.00:0.56:0.24:0.33), (DINUTUXIMAB:9:11265:3:33:1.0 0:0.75:0.21:0.33), (ZALCITABINE:9:11265:2:34:1.00:0.82:0.21:0.33), (CLOSTRIDIUM TETANI TOXOID ANTIGEN (FORMALDEHYDE INACTIVATED)\CORYNEBACTERIUM DIPHTHERIAE TOXOID ANTIGEN (FORMALDEHYDE INACTIVATED):8:11266:3:33:1.00:0.73:0.20:0.31), (CYCLOPHOSPHAMIDEDOXORUBI CINPREDNISONERITUXIMABVINCRISTINE:8:11266:6:30:1.00:0.57:0.21:0.31), (KAN AMYCIN SULFATE:8:11266:0:36:1.00:1.00:0.18:0.31), (LEVAMLODIPINE BESILATE:8:11266:1:35:1.00:0.89:0.19:0.31), (NALOXONE HYDROCHLORIDEPENTAZOCINE HYDROCHLORIDE:8:11266:9:27:1.00:0.47:0.23:0.31), (MEXAZOLAM:7:11267:1:35:1.00: 0.88:0.17:0.28), (NATURAL LATEX RUBBER:7:11267:8:28:1.00:0.47:0.20:0.28), (AMOBARBITALBELLADONNA LEAFDIMETHICONEERGOTAMINE TARTRATE:6:11268:2:34:1.00:0.75:0.15:0.25), (CAMPHOR (SYNTHETIC)\CAPSICUM OLEORESINMENTHOLMETHYL SALICYLATE:6:11268:4:32:1.00:0.60:0.16:0.25), (CAPTOPRILHYDROCHLOROTHIAZI DE:6:11268:3:33:1.00:0.67:0.15:0.25), (CHLORIDE ION:6:11268:6:30:1.00:0.50:0.17:0.25), (COCOA BUTTERPHENYLEPHRINE HYDROCHLORIDE:5:11269:0:36:1.00:1.00:0.12:0.22), (IVY EXTRACT:5:11269:2:34:1.00:0.71:0.13:0.22), (OLODATEROLTIOTROPIUM BROMIDE:5:11269:5:31:1.00:0.50:0.14:0.22), (PAMABROM:5:11269:3:33:1.00:0.63:0.13: 0.22), (POTASSIUM ASCORBATE:5:11269:0:36:1.00:1.00:0.12:0.22), (TARAXACUM OFFICINALE POLLEN:5:11269:2:34:1.00:0.71:0.13:0.22), (TERIZIDONE:5:11269:1:35:1.00:0.83:0.13:0.22), (THYMOSIN:5:11269:0:36:1.00:1.00:0.12:0.22), (DIACETYL BENZOYL LATHYROL:4:11270:3:33:1.00:0.57:0.11:0.18), (NATAMYCIN:4:11270:2:34:1.00:0.67:0.1 1:0.18), (OLARATUMAB:4:11270:1:35:1.00:0.80:0.10:0.18), (PROPOLIS WAX:4:11270:4:32:1.00:0.50:0.11:0.18), (CALCIUM PANTOTHENATENIACINAMIDEPYRIDOXINE HYDROCHLORIDE\RIBOFLAVINTHIAMINE MONONITRATE:3:11271:3:33:1.00:0.50:0.08:0.14), (OLIVE OILSOYBEAN OIL:3:11271:1:35:1.00:0.75:0.08:0.14), (OVINE DIGOXIN IMMUNE FAB:3:11271:1:35:1.00:0.75:0.08:0.14), (POMEGRANATE:3:11271:3:33:1.00:0.50:0.08:0.14), (ACETYLCHOLINE CHLORIDE:2:11272:2:34:1.00:0.50:0.06:0.10), (COAGULATION FACTOR IX HUMANCOAGULATION FACTOR VII HUMANCOAGULATION FACTORX HUMANPROTHROMBIN:1:11273:1:35:1.00:0.50:0.03:0.05), (ATOSIBAN:18:11255:1:36:1 0.00:0.95:0.33:0.49), (ROSIGLITAZONE:18:11255:15:22:1.00:0.55:0.45:0.49), (ISTRADEF YLLINE:17:11256:12:25:1.00:0.59:0.40:0.48), (PAZUFLOXACIN:16:11257:10:27:1.00:0.62: 0.37:0.46), (ALLANTOINBENZOCAINECAMPHOR (NATURAL)DIMETHICONEMENTHOL:13:11260:14:23:1.00:0.48:0.36:0.41), (CALCIUM BICARBONATE:13:11260:23:14:1.00:0.36:0.48:0.41), (LACTOPEROXIDASE BOVINELYSOZYME HYDROCHLORIDE:13:11260:9:28:1.00:0.59:0.32:0.41), (CALCIUM ACETATEMAGNESIUM CARBONATE:11:11262:1:36:1.00:0.92:0.23:0.37), (AMOBARBITAL SODIUM:10:11263:16:21:1.00:0.38:0.32:0.35), (DIETARY SUPPLEMENTFLAVOCOXID:10:11263:12:25:1.00:0.45:0.29:0.35), (METHENOLONE ACETATE:10:11263:18:19:1.00:0.36:0.34:0.35), (ACETAMINOPHENBROMISOVALDIH YDROCODEINE PHOSPHATEDIPHENHYDRAMINE SALICYLATEDYPHYLLINEMETHYLEPHEDRINE HYDROCHLORIDE, (+/−)-:9:11264:22:15:1.00:0.29:0.38:0.33), (CHLORMADINONE ACETATEMESTRANOL:9:11264:3:34:1.00:0.75:0.21:0.33), (ACETAMINOPHENGUAIF ENESINPHENYLEPHRINE HYDROCHLORIDE:8:11265:4:33:1.00:0.67:0.20:0.30), (CITICOLINE SODIUM:7:11266:5:32:1.00:0.58:0.18:0.27), (BEPOTASTINEBEPOTASTINE BESILATE:6:11267:14:23:1.00:0.30:0.21:0.24), (CITRIC ACID MONOHYDRATE\POTASSIUM CITRATE:6:11267:8:29:1.00:0.43:0.17:0.24), (FLIBANSERIN:6:11267:5:32:1.00:0.55:0.16: 0.24), (AMLODIPINEATORVASTATIN:5:11268:4:33:1.00:0.56:0.13:0.21), (KERATIN:5: 11268:6:31:1.00:0.45:0.14:0.21), (MAGNESIUM SULFATE HEPTAHYDRATE:5:11268:3:34:1.00:0.63:0.13:0.21), (NALMEFENE HYDROCHLORIDE DIHYDRATE:5:11268:0:37:1.00:1.00:0.12:0.21), (VISCUM ALBUM FRUITING TOP:5:11268:2:35:1.00:0.71:0.13:0.21), (ALPELISIB:4:11269:0:37:1.00:1.00:0.10:0.18), (G LYCERINHYPROMELLOSE 2910 (15 MPA.S)PETROLATUM OR POLYVINYL ALCOHOLPOLYETHYLENE GLYCOL 400 OR MINERAL OILPOVIDONE OR PROPYLENE GLYCOL:4:11269:0:37:1.00:1.00:0.10:0.18), (NECITUMUMAB:4:11269:0:37:1.00:1.00:0.1 0:0.18), (PSORALEN:4:11269:28:9:1.00:0.13:0.31:0.18), (COLCHICINEPROBENECID:3:1 1270:1:36:1.00:0.75:0.08:0.14), (MALIC ACID:3:11270:5:32:1.00:0.38:0.09:0.14), (MEGLUMINE:3:11270:1:36:1.00:0.75:0.08:0.14), (HYALURONIC ACIDLIDOCAINE HYDROCHLORIDE:2:11271:5:32:1.00:0.29:0.06:0.10), (WHEAT GLUTEN:2:11271:2:35:1.00:0.50:0.05:0.10), (INTERFERON BETA-1B:322:10950:0:38:1.00:1.00:0.89:0.94), (FLUPHENAZINE DECANOATE:19:11253:7:31:1.00:0.73:0.38:0.50), (PODOFILOX:19:11253:16:22:1.00:0.54: 0.46:0.50), (ACLIDINIUM BROMIDEFORMOTEROL FUMARATE:18:11254:9:29:1.00:0.67:0.38:0.49), (PHENYLEPHRINE HYDROCHLORIDEPROMETHAZINE HYDROCHLORIDE:14:11258:14:24:1.00:0.50:0.37:0.42), (AMPHETAMINEDEXTROAM PHETAMINE:12:11260:12:26:1.00:0.50:0.32:0.39), (CAMPHOR (SYNTHETIC)\EUCALYPTUS OILMENTHOL:12:11260:1:37:1.00:0.92:0.24:0.39), (PHENYLEPHRINE HYDROCHLORIDEWITCH HAZEL:9:11263:24:14:1.00:0.27:0.39:0.32), (ARFORMOTEROL:8:11264:5:33:1.00:0.62:0.20:0.30), (ATENOLOLNIFEDIPINE:7:11265:1:37:1.00:0.88:0.16:0.27), (THYROGLOBUL IN:7:11265:5:33:1.00:0.58:0.18:0.27), (ACETYLCYSTEINEBENZALKONIUM CHLORIDETUAMINOHEPTANE SULFATE:6:11266:12:26:1.00:0.33:0.19:0.24), (METHYLPREDNISOLONE HEMISUCCINATE:6:11266:2:36:1.00:0.75:0.14:0.24), (OXYPERTINE:6:11266:5:33:1.00:0 0.55:0.15:0.24), (ESTRADIOLPROGESTERONE:5:11267:2:36:1.00:0.71:0.12:0.21), (FLUD IAZEPAM:5:11267:0:38:1.00:1.00:0.12:0.21), (NEISSERIA MENINGITIDIS GROUP A CAPSULAR OLIGOSACCHARIDE DIPHTHERIA CRM197 PROTEIN CONJUGATE ANTIGEN\NEISSERIA MENINGITIDIS GROUP C CAPSULAR OLIGOSACCHARIDE DIPHTHERIA CRM197 PROTEIN CONJUGATE ANTIGEN\NEISSERIA MENINGITIDIS GROUP W-135 CAPSULAR OLIGOSACCHARIDE DIPHTHERIA CRM197 PROTEIN CONJUGATE ANTI:5:11267:3:35:1.00:0.63:0.13:0.21), (NIACINAMIDEPYRIDOXINE\RIBOFLAVINT HIAMINE:5:11267:11:27:1.00:0.31:0.16:0.21), (ACETAMINOPHENIBUPROFEN:4:11268: 0:38:1.00:1.00:0.10:0.17), (CEPHALOTHIN:4:11268:0:38:1.00:1.00:0.10:0.17), (CROFELE MER:4:11268:10:28:1.00:0.29:0.13:0.17), (FLUCLOXACILLIN SODIUM ANHYDROUS:4:11268:0:38:1.00:1.00:0.10:0.17), (INFLUENZA A VIRUS A/CALIFORNIA/7/2009 X-179A (H1N1) ANTIGEN (FORMALDEHYDE INACTIVATED)\INFLUENZA A VIRUS A/TEXAS/50/2012 X-223A (H3N2) ANTIGEN (FORMALDEHYDE INACTIVATED)\INFLUENZA B VIRUS B/BRISBANE/60/2008 ANTIGEN (FORMALDEHYDE INACTIVATED)\INFLUENZA B VIRUS B/MASSACHUSETTS/2/2012 ANTIGEN (FORMALDE:4:11268:4:34:1.00:0.50:0.11:0.17), (ACETAMINOPHEN\ASPIRIN\CAFFEINE\SALICYLAMIDE:3:11269:1:37:1.00:0.75:0.08:0.14), (AZASETRON:3:11269:3:35:1.00:0.50:0.08:0.14), (DEXTROSE MONOHYDRATEDEXTROSE MONOHYDRATEDOBUTAMINE HYDROCHLORIDEDOBUTAMINE HYDROCHLORIDE:3:11269:1:37:1.00:0.75:0.08:0.14), (DEXTROSEFRUCTOSEPHOSPH ORIC ACID:3:11269:2:36:1.00:0.60:0.08:0.14), (LORLATINIB:3:11269:1:37:1.00:0.75:0.08:0.14), (OPICINUMAB:3:11269:1:37:1.00:0.75:0.08:0.14), (JOSAMYCIN:2:11270:2:36:1.00:0.50: 0.05:0.10), (EPTIFIBATIDE:54:11217:0:39:1.00:1.00:0.58:0.73), (ESTROGENSMETHYL TESTOSTERONE:23:11248:19:20:1.00:0.55:0.53:0.54), (ELOSULFASE ALFA:22:11249:7:32:1.00:0.76:0.41:0.53), (SUCCINYLATED GELATIN:14:11257:2:37:1.00:0.88:0.27:0.42), (PHENTOLAMINE:13:11258:6:33:1.00:0.68: 0.28:0.40), (CYSTEAMINE BITARTRATE:12:11259:7:32:1.00:0.63:0.27:0.38), (BACLOFENCYCLOBENZAPRINE\DICLOFENACGABAPENTINTETRACAINE:11:11260:11:28:1.00:0.50:0.28:0.36), (AMCIN ONIDE:9:11262:2:37:1.00:0.82:0.20:0.32), (LYSINE HYDROCHLORIDE:9:11262:6:33:1.00:0.60:0.21:0.32), (INTERFERON ALFA-N1:8:11263:19:20:1.00:0.30:0.29:0.29), (CAT'S CLAW:7:11264:4:35:1.00:0.64:0.17:0.26), (CHOLINE:7:11264:3:36:1.00:0.70:0.16:0.26), (NIPRADILOL:7:11264:6:33:1.00:0.54:0.18:0.26), (SUPROFEN:7:11264:4:35:1.00:0.64:0.17: 0.26), (CEDIRANIB:6:11265:1:38:1.00:0.86:0.14:0.24), (CITRIC ACID MONOHYDRATEDEXTROSE\POTASSIUM CATION\SODIUM CHLORIDE:6:11265:0:39:1.00:1.00:0.13:0.24), (DICHLORPHENAMIDE:6:11265:19:20:1.0 0:0.24:0.23:0.24), (GENTIAN VIOLET:6:11265:19:20:1.00:0.24:0.23:0.24), (OXAZOLAM:6:11265:0:39:1.00:1.00:0.13:0.24), (PICLOXYDINE DIHYDROCHLORIDE:6:11265:0:39:1.00:1.00:0.13:0.24), (PROPANTHELINE:6:11265:5:3 4:1.00:0.55:0.15:0.24), (ACETAMINOPHEN\ASPIRINCHLORPHENIRAMINE MALEATEDEXTROMETHORPHAN HYDROBROMIDEDIPHENHYDRAMINE HYDROCHLORIDEDOXYLAMINE SUCCINATEPHENYLEPHRINE BITARTRATEPHENYLEPHRINE HYDROCHLORIDE:5:11266:0:39:1.00:1.00:0.11:0.20), (DEXAMETHASONE PALMITATE:5:11266:0:39:1.00:1.00:0.11:0.20), (LANOLINMINERAL OILPETROLATUM:5:11266:16:23:1.00:0.24:0.18:0.20), (URAPIDIL HYDROCHLORIDE:5:11266:1:38:1.00:0.83:0.12:0.20), (GLYCERINHYPROMELLOSE 2910 (15 MPA.S)POLYETHYLENE GLYCOL 400PROPYLENE GLYCOL:4:11267:3:36:1.00:0.57:0.10:0.17), (LEVOMETHADONE:4:11267:1:38:1.00:0.80: 0.10:0.17), (WHEAT GERM OIL:4:11267:2:37:1.00:0.67:0.10:0.17), (BIOFLAVONOIDS:3:11268:2:37:1.00:0.60:0.08:0.13), (CEFDITOREN:3:11268:1:38:1.00:0.75:0.07:0.13), (ESCIN:3:11268:1:38:1.00:0.75:0.0 7:0.13), (THIORIDAZINE HYDROCHLORIDE:3:11268:2:37:1.00:0.60:0.08:0.13), (INDOCYANINE GREEN:2:11269:1:38:1.00:0.67:0.05:0.09), (VIGABATRIN:231:11039:0:40:1.00:1.00:0.85: 0.92), (IRONMINERALSVITAMINS:23:11247:1:39:1.00:0.96:0.37:0.53), (IFENPRODIL TARTRATE:21:11249:12:28:1.00:0.64:0.43:0.51), (QUERCUS SALICINA STEM:17:11253:12:28:1.00:0.59:0.38:0.46), (ACETAMINOPHENDICHLORALPHENAZO NEISOMETHEPTENE HYDROCHLORIDE:15:11255:7:33:1.00:0.68:0.31:0.43), (ASPIRINCARISOPRODOL:12:1 1258:3:37:1.00:0.80:0.24:0.38), (HYDROCORTISONE PROBUTATE:12:11258:26:14:1.00:0.32:0.46:0.38), (ASPIRINDIHYDROXYALUMINUM AMINOACETATEMAGNESIUM CARBONATE:11:11259:10:30:1.00:0.52:0.27:0.35), (NUSINERSEN:10:11260:29:11:1.00:0.26:0.48:0.33), (RIMABOTULINUMTOXINB:10:11260:3:37:1.00:0.77:0.21:0.33), (.BETA.-HYDROXYISOVALERIC ACIDARGININEGLUTAMINE:9:11261:21:19:1.00:0.30:0.32:0.31), (CAFFEINEISOMET HEPTENEMETHIMAZOLE:9:11261:13:27:1.00:0.41:0.25:0.31), (SENNOSIDE ASENNOSIDE BSENNOSIDES:9:11261:7:33:1.00:0.56:0.21:0.31), (VALRUBICIN:9:11261:13:27:1.00:0.4 1:0.25:0.31), (FRANGULA PURSHIANA BARK:8:11262:5:35:1.00:0.62:0.19:0.29), (PROCYCLIDINE HYDROCHLORIDE:8:11262:4:36:1.00:0.67:0.18:0.29), (ISOFLAVONES SOY:7:11263:16:24:1.00:0.30:0.23:0.26), (PIPAMPERONE HYDROCHLORIDE:7:11263:0:40:1.00:1.00:0.15:0.26), (SHARK LIVER OIL:7:11263:5:35:1.00:0.58:0.17:0.26), (THIABENDAZOLE:7:11263:3:37:1.00:0.70:0.16:0 0.26), (TRIFLURIDINE:7:11263:1:39:1.00:0.88:0.15:0.26), (MEFLOQUINE HYDROCHLORIDE:6:11264:3:37:1.00:0.67:0.14:0.23), (TESTOSTERONE PROPIONATE:6:11264:0:40:1.00:1.00:0.13:0.23), (DROTRECOGIN ALFA (ACTIVATED):5:11265:3:37:1.00:0.63:0.12:0.20), (CALCIUM SULFATE:4:11266:2:38:1.00:0.67:0.10:0.17), (CLIDINIUM BROMIDE:4:11266:5:35:1.00:0.44:0.10:0.17), (DROTAVERINE:4:11266:0:40:1.00:1.00:0.09:0.17), (FUMARIC ACID:4:11266:1:39:1.00:0.80:0.09:0.17), (LACTULOSEPARAFFIN:4:11266:1:39:1.00:0.80: 0.09:0.17), (NETUPITANTPALONOSETRON HYDROCHLORIDE:4:11266:0:40:1.00:1.00:0.09:0.17), (PHENOLPHTHALEIN:4:11266:0:4 0:1.00:1.00:0.09:0.17), (TALNIFLUMATE:4:11266:0:40:1.00:1.00:0.09:0.17), (ACETAMI NOPHEN AND OR DEXTROMETHORPHAN AND OR DIPHENHYDRAMINE AND OR GUAIFENESIN AND OR PHENYLEPHRINE AND OR PSEUDOEPHEDRINE:3:11267:4:36:1.00:0.43:0.08:0.13), (ARTICAINE HYDROCHLORIDEEPINEPHRINE BITARTRATE:3:11267:3:37:1.00:0.50:0.08:0.13), (ASCORBIC ACIDFERROUS ASPARTO GLYCINATEFERROUS FUMARATEFOLIC ACIDNIACIN:3:11267:3:37:1.00:0.50:0.08:0.13), (ESTRADIOLESTRIOLESTRONE:3:11 267:1:39:1.00:0.75:0.07:0.13), (GLUCUROLACTONE:3:11267:1:39:1.00:0.75:0.07:0.13), (HAWTHORN LEAF WITH FLOWER:3:11267:4:36:1.00:0.43:0.08:0.13), (METAPROTERENOL:2:11268:2:38:1.00:0.5 0:0.05:0.09), (PAROMOMYCIN SULFATE:2:11268:2:38:1.00:0.50:0.05:0.09), (DACLATASVIR DIHYDROCHLORIDE:178:11091:7:34:1.00:0.96:0.84:0.90), (FOLLITROPIN:81:11188:0:41: 1.00:1.00:0.66:0.80), (QUINAGOLIDE HYDROCHLORIDE:17:11252:18:23:1.00:0.49:0.43:0.45), (SAQUINAVIR:17:11252:2:39:1.00:0.89:0.30:0.45), (CLOCORTOLONE PIVALATE:15:11254:10:31:1.00:0.60:0.33:0.42), (TOFOGLIFLOZIN:11:11258:2:39:1.00:0.85:0.22:0.35), (BUNAZOSIN HYDROCHLORIDE:10:11259:2:39:1.00:0.83:0.20:0.33), (CIPROFLOXACIN HYDROCHLORIDEHYDROCORTISONE:9:11260:5:36:1.00:0.64:0.20:0.31), (ECHINACEA PURPUREA:9:11260:4:37:1.00:0.69:0.20:0.31), (ACETAMINOPHEN\ASPIRIN:8:11261:6:3 5:1.00:0.57:0.19:0.28), (EPACADOSTAT:7:11262:17:24:1.00:0.29:0.23:0.25), (METHYL SALICYLATE:7:11262:1:40:1.00:0.88:0.15:0.25), (PIROXICAM BETADEX:7:11262:1:40:1.00:0.88:0.15:0.25), (BELLADONNA:5:11264:1:40:1.00:0.83:0.11:0.20), (TRICHLOROACETIC ACID:5:11264:3:38:1.00:0.63:0.12:0.20), (SUBTILIS:4:11265:2:39:1.00:0.67:0.09:0.16), (GLUCONATE:4:11265:24:17:1.00:0.14:0.19: 0.16), (HEPATITIS A VIRUS STRAIN HM175 ANTIGEN (FORMALDEHYDE INACTIVATED)HEPATITIS B VIRUS SUBTYPE ADW2 HBSAG SURFACE PROTEIN ANTIGEN:4:11265:1:40:1.00:0.80:0.09:0.16), (NECUPARANIB:4:11265:0:41:1.00:1.00:0.0 9:0.16), (UBENIMEX:4:11265:4:37:1.00:0.50:0.10:0.16), (CARBON DIOXIDE:3:11266:15:26:1.00:0.17:0.10:0.13), (POTASSIUM PERMANGANATE:3:11266:1:40:1.00:0.75:0.07:0.13), (INOSINE PRANOBEX:2:11267:5:36:1.00:0.29:0.05:0.09), (TECHNETIUM TC-99M:1:11268:1:40:1.00:0.50:0.02:0.05), (MESTRANOLNORETHINDRONE:25:11243:17:2 5:1.00:0.60:0.50:0.54), (ETHINYL ESTRADIOLFERROUS FUMARATENORETHINDRONE:22:11246:17:25:1.00:0.56:0.47:0.51), (ALUMINUM CHLORIDE:18:11250:9:33:1.00:0.67:0.35:0.46), (DEXTROMETHORPHANDEXTROMET HORPHAN HYDROBROMIDEGUAIFENESIN:17:11251:9:33:1.00:0.65:0.34:0.45), (DEXTRAN SULFATE SODIUM SALT:13:11255:8:34:1.00:0.62:0.28:0.38), (DEXAMETHASONENEOMYCINPOLYMYXI N B SULFATE:12:11256:14:28:1.00:0.46:0.30:0.36), (POTASSIUM PERCHLORATE:11:11257:1:41:1.00:0.92:0.21:0.34), (PRUCALOPRIDE:10:11258:6:36:1.0 0:0.63:0.22:0.32), (ELIGLUSTAT:9:11259:1:41:1.00:0.90:0.18:0.30), (POTASSIUM CITRATE\SODIUM CITRATE\ZINC:9:11259:4:38:1.00:0.69:0.19:0.30), (BROMPHENIRAMINE MALEATEPSEUDOEPHEDRINE HYDROCHLORIDE:8:11260:4:38:1.00:0.67:0.17:0.28), (EPERISONE:8:11260:0:42:1.00:1.00:0.16:0.28), (GLIQUIDONE:8:11260:4:38:1.00:0.67:0.17:0.28), (POLYETHYLENE GLYCOL 3350\POTASSIUM CHLORIDE\SODIUM BICARBONATE\SODIUM CHLORIDE\SODIUM SULFATE\SODIUM SULFATE ANHYDROUS:8:11260:3:39:1.00:0.73:0.17:0.28), (RIMANTADINE HYDROCHLORIDE:8:11260:6:36:1.00:0.57:0.18:0.28), (BETAINE:7:11261:6:36:1.00:0.54: 0.16:0.25), (DIMETHYL SULFONEGLUCOSAMINE SULFATE:7:11261:6:36:1.00:0.54:0.16:0.25), (AMOROLFINE:6:11262:5:37:1.00:0.55:0.14: 0.22), (LITHIUM CITRATE:6:11262:2:40:1.00:0.75:0.13:0.22), (CERTOLIZUMAB:5:11263:7:35:1.00:0.42:0.13:0.19), (QUINIDINE GLUCONATE:5:11263:35:7:1.00:0.13:0.42:0.19), (TETANUS ANTITOXIN:5:11263:2:40:1.00:0.71:0.11:0.19), (ACETAMINOPHEN\ASPIRINSALICYL AMIDE:4:11264:6:36:1.00:0.40:0.10:0.16), (CALCIUMCHOLECALCIFEROLMAGNESIU M\ZINC:4:11264:3:39:1.00:0.57:0.09:0.16), (TOBACCO LEAF:4:11264:0:42:1.00:1.00:0.09:0.16), (CALCIUM PANTOTHENATENIACINAMIDEPYRIDOXINE HYDROCHLORIDE\RIBOFLAVINTHIAMINE HYDROCHLORIDE:3:11265:6:36:1.00:0.33:0.08:0.13), (CEFTIBUTEN DIHYDRATE:3:11265:1:41:1.00:0.75:0.07:0.13), (IBUPROFEN SODIUM:3:11265:3:39:1.00:0.50:0.07:0.13), (METHYLPREDNISOLONE SODIUM PHOSPHATE:3:11265:1:41:1.00:0.75:0.07:0.13), (PECTIN:3:11265:1:41:1.00:0.75:0.07:0.1 3), (PIPERAZINE:3:11265:1:41:1.00:0.75:0.07:0.13), (OMBITASVIR:2:11266:2:40:1.00:0.5 0:0.05:0.09), (LEVOBUPIVACAINE:36:11231:13:30:1.00:0.73:0.55:0.63), (RIMEXOLONE: 22:11245:1:42:1.00:0.96:0.34:0.51), (TENECTEPLASE:18:11249:9:34:1.00:0.67:0.35:0.46), (ETIDRONATE DISODIUM:14:11253:0:43:1.00:1.00:0.25:0.39), (OLODATEROL:11:11256:7:36:1.00:0.61 0.23:0.34), (ROXADUSTAT:10:11257:2:41:1.00:0.83:0.20:0.32), (METHYLNALTREXON E:9:11258:5:38:1.00:0.64:0.19:0.30), (ACETAMINOPHENDIPHENHYDRAMINEDIPHEN HYDRAMINE CITRATE:8:11259:5:38:1.00:0.62:0.17:0.27), (BISMUTH:8:11259:3:40:1.00:0.73:0.17:0.27), (METHYLENEDIOXYMETHAMPHETAMINE:7:11260:5:38:1.00:0.58:0.16:0.25), (DEXA METHASONE SODIUM PHOSPHATENEOMYCIN SULFATE:6:11261:0:43:1.00:1.00:0.12:0.22), (PERIFOSINE:6:11261:1:42:1.00:0.86:0.13:0 0.22), (ALBUTEROLGUAIFENESIN:5:11262:36:7:1.00:0.12:0.42:0.19), (ESOMEPRAZOLE STRONTIUM:5:11262:2:41:1.00:0.71:0.11:0.19), (ALUMINUM HYDROXIDEMAGNESIUM TRISILICATE:4:11263:2:41:1.00:0.67:0.09:0.16), (HAEMOPHILUS B CONJUGATE VACCINE:4:11263:0:43:1.00:1.00:0.09:0.16), (PANCURONIUM BROMIDE:4:11263:0:43:1.00:1.00:0.09:0.16), (CHLORELLA VULGARIS:3:11264:2:41:1.00:0.60:0.07:0.12), (ENDOSTATIN:3:11264:1:42:1.00:0.75:0.0 7:0.12), (CLINDAMYCIN PALMITATE HYDROCHLORIDE:1:11266:3:40:1.00:0.25:0.02:0.04), (EPOPROSTENOL SODIUM:136:11130:0:44:1.00:1.00:0.76:0.86), (POLYETHYLENE GLYCOL 400:35:11231:17:27:1.00:0.67:0.56:0.61), (BUPROPION HYDROBROMIDE:18:11248:12:32:1.00:0.60:0.36:0.45), (CEFTERAM PIVOXIL:16:11250:21:23:1.00:0.43:0.41:0.42), (FOSAMPRENAVIR:16:11250:1:43:1.00:0.94:0.27:0.42), (CROTALIDAE POLYVALENT IMMUNE FAB (OVINE):15:11251:25:19:1.00:0.38:0.44:0.41), (SEA WATER:12:11254:6:38:1.00:0.67:0.24:0.35), (NALDEMEDINE:11:11255:11:33:1.00:0.50:0 0.25:0.33), (AMITRIPTYLINE HYDROCHLORIDEPERPHENAZINE:10:11256:0:44:1.00:1.00:0.19:0.31), (CAPSICUM:8: 11258:16:28:1.00:0.33:0.22:0.27), (LECITHIN\SODIUM BICARBONATE\SODIUM PHOSPHATE, MONOBASIC:8:11258:7:37:1.00:0.53:0.18:0.27), (CREATININE:7:11259:13:31:1.00:0.35 0.18:0.24), (CLIDINIUM:6:11260:0:44:1.00:1.00:0.12:0.21), (HYOSCYAMINE SULFATEMETHENAMINEMETHYLENE BLUE\SODIUM PHOSPHATE, MONOBASIC:6:11260:2:42:1.00:0.75:0.13:0.21), (CALCIUM CHLORIDEMAGNESIUM CHLORIDE\POTASSIUM CHLORIDE\POTASSIUM PHOSPHATE, DIBASIC\POTASSIUM PHOSPHATE, MONOBASIC\SODIUM CHLORIDE:5:11261:2:42:1.00:0.71:0.11:0.19), (KONJAC MANNAN:5:11261:3:41:1.00:0.63:0.11:0.19), (NEOMYCINPOLYMYXIN:5:11261:5:39:1.0 0:0.50:0.11:0.19), (CABIRALIZUMAB:4:11262:0:44:1.00:1.00:0.08:0.15), (CEFOPERAZONE SODIUMSULBACTAM:4:11262:3:41:1.00:0.57:0.09:0.15), (TASELISIB:4:11262:2:42:1.00: 0.67:0.09:0.15), (GEMIFLOXACIN MESYLATE:3:11263:1:43:1.00:0.75:0.07:0.12), (PIRENZEPINE HYDROCHLORIDE:3:11263:3:41:1.00:0.50:0.07:0.12), (SAVOLITINIB:2:11264:2:42:1.00: 0.50:0.05:0.08), (IFOSFAMIDE:664:10601:0:45:1.00:1.00:0.94:0.97), (CODEINE PHOSPHATEDICLOFENAC SODIUM:25:11240:13:32:1.00:0.66:0.44:0.53), (HYDROXYPROPYL CELLULOSE:25:11240:15:30:1.00:0.63:0.45:0.53), (ALCOHOLALUMINUM CHLORIDE:19:11246:10:35:1.00:0.66:0.35:0.46), (CYANOCOBALAMINDICLOFENAC SODIUMPYRIDOXINE HYDROCHLORIDETHIAMINE MONONITRATE:19:11246:12:33:1.00:0.61:0.37:0.46), (BERBERINEGERANIUM:18:1124 7:4:41:1.00:0.82:0.31:0.44), (HALCINONIDE:18:11247:22:23:1.00:0.45:0.44:0.44), (CYAN OCOBALAMINPYRIDOXINE HYDROCHLORIDETHIAMINE DISULFIDE:15:11250:10:35:1.00:0.60:0.30:0.40), (THYROTROPIN ALFA:14:11251:3:42:1.00:0.82:0.25:0.38), (BENZOCAINERESORCINOL:12:11253:13:32: 1.00:0.48:0.27:0.35), (CYCLOPHOSPHAMIDECYTARABINEDEXAMETHASONEDOX ORUBICINMETHOTREXATEVINCRISTINE:12:11253:30:15:1.00:0.29:0.44:0.35), (DOC USATE SODIUMSENNOSIDE ASENNOSIDE B:12:11253:12:33:1.00:0.50:0.27:0.35), (HYDROCHLORIC ACID:12:11253:20:25:1.00:0.38:0.32:0.35), (FUROSEMIDE\POTASSIUM CHLORIDE:10:11255:2:43:1.00:0.83:0.19:0.31), (TANDOSPIRONE CITRATE:10:11255:4:41:1.00:0.71:0.20:0.31), (ACETAMINOPHENPSEUDOEPHEDRINE HYDROCHLORIDE:9:11256:15:30:1.00:0.38:0.23:0.29), (DEMECLOCYCLINE HYDROCHLORIDE:9:11256:2:43:1.00:0.82:0.17:0.29), (DEXAMETHASONE SODIUM PHOSPHATEMOXIFLOXACIN HYDROCHLORIDE:9:11256:3:42:1.00:0.75:0.18:0.29), (SHARK CARTILAGESHARK, UNSPECIFIED:9:11256:11:34:1.00:0.45:0.21:0.29), (BUFORMIN:8:11257:31:14:1.00:0.21: 0.36:0.26), (LAVENDER OIL:8:11257:9:36:1.00:0.47:0.18:0.26), (SEBELIPASE ALFA:8:11257:8:37:1.00:0.50:0.18:0.26), (SULFAMETHAZINE:8:11257:3:42:1.00:0.73:0.1 6:0.26), (FERROUS FUMARATEFOLIC ACIDVITAMIN B COMPLEX:7:11258:2:43:1.00:0.78:0.14:0.24), (FERROUS SUCCINATE:7:11258:1:44:1.00:0.88:0.14:0.24), (ACRIVASTINE:5:11260:4:41:1.00:0.56:0 0.11:0.18), (RALTITREXED:5:11260:7:38:1.00:0.42:0.12:0.18), (BISULEPIN HYDROCHLORIDE:4:11261:0:45:1.00:1.00:0.08:0.15), (CEFOZOPRAN:4:11261:5:40:1.00: 0.44:0.09:0.15), (CLINDAMYCIN PHOSPHATETRETINOIN:4:11261:3:42:1.00:0.57:0.09:0.15), (DINOPROST:4:11261:0:45: 1.00:1.00:0.08:0.15), (GEDATOLISIB:4:11261:0:45:1.00:1.00:0.08:0.15), (HUMAN COAGULATION FACTOR VIII/VON WILLEBRAND FACTOR COMPLEX:4:11261:1:44:1.00:0.80:0.08:0.15), (INTERFERON GAMMA:4:11261:4:41:1.00:0.50:0.09:0.15), (PARECOXIB:4:11261:0:45:1.00:1.00:0.08:0.), (ENTINOSTAT:3:11262:1:44:1.00:0.75:0.06:0.12), (LACTOBACILLUS REUTERI:3:11262:2:43:1.00:0.60:0.07:0.12), (METHOXYPHENAMINE:3:11262:1:44:1.00: 0.75:0.06:0.12), (PHENIRAMINE MALEATE:3:11262:0:45:1.00:1.00:0.06:0.12), (JWH-018:2:11263:7:38:1.00:0.22:0.05:0.08), (THIAMINE MONONITRATE:2:11263:1:44:1.00:0.67:0.04:0.08), (THIOGUANINE:50:11214:14:32:1.00: 0.78:0.61:0.68), (SOFALCONE:22:11242:11:35:1.00:0.67:0.39:0.49), (BROMPERIDOL:19: 11245:13:33:1.00:0.59:0.37:0.45), (POLYETHYLENE GLYCOL 3350POTASSIUM CHLORIDE\SODIUM BICARBONATE\SODIUM CHLORIDE\SODIUM SULFATE:19:11245:0:46:1.00:1.00:0.29:0.45), (VORAPAXAR SULFATE:19:11245:24:22:1.00:0.44:0.46:0.45), (LATANOPROSTTIMOLOL:16:11248:28: 18:1.00:0.36:0.47:0.41), (THROMBOPOIETIN:16:11248:16:30:1.00:0.50:0.35:0.41), (CHL ORMADINONE ACETATEETHINYL ESTRADIOL:14:11250:16:30:1.00:0.47:0.32:0.38), (TISAGENLECLEUCEL:12:11252:20:26: 1.00:0.38:0.32:0.34), (ACETAMINOPHENCHLORPHENIRAMINE MALEATEPSEUDOEPHEDRINE HYDROCHLORIDE:11:11253:23:23:1.00:0.32:0.32:0.32), (ASPIRIN\CAFFEINESALICYL AMIDE:11:11253:40:6:1.00:0.22:0.65:0.32), (BLESELUMAB:9:11255:40:6:1.00:0.18:0.60: 0.28), (NAFRONYL OXALATE:8:11256:1:45:1.00:0.89:0.15:0.26), (QUININE HYDROCHLORIDE:8:11256:1:45:1.00:0.89:0.15:0.26), (ALTHIAZIDE:6:11258:0:46:1.00:1 0.00:0.12:0.21), (CHLORPHENIRAMINE MALEATEPHENYLEPHRINE HYDROCHLORIDE:6:11258:4:42:1.00:0.60:0.13:0.21), (CLOPIDOGREL HYDROCHLORIDE:6:11258:1:45:1.00:0.86:0.12:0.21), (CLOTRIMAZOLE OR MICONAZOLE NITRATE:6:11258:0:46:1.00:1.00:0.12:0.21), (COPPER GLUCONATE:6:11258:3:43:1.00:0.67:0.12:0.21), (MOLYBDENUM:6:11258:1:45:1.00:0.86: 0.12:0.21), (ACETAMINOPHENBELLADONNA LEAFCAFFEINE\MORPHINE:5:11259:3:43:1.00:0.63:0.10:0.18), (LEDIPASVIR:5:11259:0: 46:1.00:1.00:0.10:0.18), (SODIUM PHOSPHATE, DIBASIC, ANHYDROUS\SODIUM PHOSPHATE, MONOBASIC, MONOHYDRATE:5:11259:3:43:1.00:0.63:0.10:0.18), (CANDESARTAN CILEXETILFELODIPINE:4:11260:4:42:1.00:0.50:0.09:0.15), (CITRIC ACID MONOHYDRATEDEXTROSE\SODIUM CITRATE:4:11260:1:45:1.00:0.80:0.08:0.15), (ASCORBIC ACIDFERROUS SULFATEFOLIC ACIDPYRIDOXINE HYDROCHLORIDE\RIBOFLAVINTHIAMINE MONONITRATE:24:11239:11:36:1.00:0.69:0.40:0.51), (AMPHETAMINE SULFATE:23:11240:15:32:1.00:0.61:0.42:0.49), (MORPHINE\MORPHINE HYDROCHLORIDE\MORPHINE SULFATE\MORPHINE TARTRATE:21:11242:23:24:1.00:0.48:0.47:0.47), (TALIGLUCERASE ALFA:17:11246:10:37:1.00:0.63:0.31:0.42), (MECLOZINE DIHYDROCHLORIDE:16:11247:25:22:1.00:0.39:0.42:0.41), (DEPRODONE PROPIONATE:12:11251:6:41:1.00:0.67:0.23:0.34), (REBOXETINE MESYLATE:10:11253:3:44:1.00:0.77:0.19:0.30), (DIHYDROERGOTAMINE:9:11254:1:46: 1.00:0.90:0.16:0.28), (GIMERACILOTERACIL POTASSIUMTEGAFUR:9:11254:1:46:1.00:0.90:0.16:0.28), (STREPTOKINASE:8:11255:5: 42:1.00:0.62:0.16:0.25), (VALPROATE MAGNESIUM:8:11255:2:45:1.00:0.80:0.15:0.25), (APRACLONIDINE HYDROCHLORIDE:7:11256:1:46:1.00:0.88:0.13:0.23), (HYDROXOCOBALAMIN HYDROCHLORIDE:7:11256:2:45:1.00:0.78:0.13:0.23), (NAPROXEN SODIUMPSEUDOEPHEDRINE HYDROCHLORIDE:7:11256:0:47:1.00:1.00:0.13:0.23), (CICLETANINE HYDROCHLORIDE:5:11258:0:47:1.00:1.00:0.10:0.18), (SOY ISOFLAVONES:4:11259:6:41:1.00:0.40:0.09:0.15), (ALDOSTERONE:3:11260:2:45:1.00:0.60:0.06:0.11), (ASCORBIC ACIDERGOCALCIFEROLRETINOLTOCOPHEROL:3:11260:1:46:1.00:0.75:0.06:0.11), (AZTREONAM LYSINE:124:11138:1:47:1.00:0.99:0.73:0.84), (TETRACAINE:41:11221:5:43:1.00:0.89:0.4 9:0.63), (SODIUM CARBONATE:25:11237:1:47:1.00:0.96:0.35:0.51), (COPPER OR LEVONORGESTREL:23:11239:6:42:1.00:0.79:0.35:0.49), (BIXALOMER:22:11240:19:29:1.00:0.54:0.43:0.48), (CODEINE PHOSPHATEPROMETHAZINE:21:11241:19:29:1.00:0.53:0.42:0.47), (GUSPERIMUS TRIHYDROCHLORIDE:20:11242:28:20:1.00:0.42:0.50:0.45), (HERBSROOTS:20:11242:7: 41:1.00:0.74:0.33:0.45), (FINGOLIMOD:16:11246:7:41:1.00:0.70:0.28:0.40), (EPHEDRINE HYDROCHLORIDEGUAIFENESIN:13:11249:40:8:1.00:0.25:0.62:0.35), (ESTRADIOLES TRIOL:12:11250:3:45:1.00:0.80:0.21:0.33), (FLUPENTIXOL DECANOATE:12:11250:8:40:1.00:0.60:0.23:0.33), (TIANEPTINE SODIUM:12:11250:6:42:1.00:0.67:0.22:0.33), (MELPHALAN HYDROCHLORIDE:11:11251:4:44:1.00:0.73:0.20:0.31), (ACETAMINOPHEN\CAFFEINE ISOMETHEPTENE MUCATE:10:11252:1:47:1.00:0.91:0.18:0.29), (PROTEASE:10:11252:3:45:1.00:0.77:0.18 0.29), (TOLBUTAMIDE:10:11252:2:46:1.00:0.83:0.18:0.29), (AMITRIPTYLINEPERPHEN AZINE:9:11253:10:38:1.00:0.47:0.19:0.27), (CAJUPUT OILEUCALYPTUS OILPEPPERMINT OIL:9:11253:30:18:1.00:0.23:0.33:0.27), (CINEPAZIDE MALEATE:8:11254:1:47:1.00:0.89:0.15:0.25), (AMBENONIUM CHLORIDE:6:11256:1:47:1.00:0.86:0.11:0.20), (MENINGOCOCCAL GROUP B VACCINE:6:11256:2:46:1.00:0.75:0.12:0.20), (RANITIDINE BISMUTH CITRATE:6:11256:3:45:1.00:0.67:0.12:0.20), (ANTIHEMOPHILIC FACTOR HUMANFIBRINOGEN HUMANFACTOR XIII CONCENTRATE (HUMAN)VON WILLEBRAND FACTOR HUMAN:5:11257:1:47:1.00:0.83:0.10:0.17), (CADEXOMER IODINE:5:11257:1:47:1.00:0.83:0.10:0.17), (INFLUENZA A VIRUS A/CALIFORNIA/7/2009 X-181 (H1N1) ANTIGEN (FORMALDEHYDE INACTIVATED)\INFLUENZA A VIRUS A/VICTORIA/210/2009 X-187 (H3N2) ANTIGEN (FORMALDEHYDE INACTIVATED)\INFLUENZA B VIRUS B/BRISBANE/60/2008 ANTIGEN (FORMALDEHYDE INACTIVATED)\INFLUENZA VIRUS VACCINE:5:11257:2:46:1.00:0.71:0.10:0.17), (CITRIC ACID MONOHYDRATEMAGNESIUM OXIDE\SODIUM PICOSULFATE:4:11258:1:47:1.00:0.80:0.08:0.14), (ERISMODEGIB:4:11258:0:48:1.00:1.0 0:0.08:0.14), (PROTIONAMIDE:2:11260:2:46:1.00:0.50:0.04:0.08), (CALCIUM CHLORIDEDEXTROSE MONOHYDRATEMAGNESIUM CHLORIDE\SODIUM CHLORIDE\SODIUM LACTATE:41:11220:15:34:1.00:0.73:0.55:0.63), (TETRACAINE HYDROCHLORIDE:41:11220:18:31:1.00:0.69:0.57:0.63), (MITIGLINIDE CALCIUM HYDRATEVOGLIBOSE:40:11221:28:21:1.00:0.59:0.66:0.62), (LEVOFLOXACIN HYDROCHLORIDE:29:11232:9:40:1.00:0.76:0.42:0.54), (FLUOROURACILIRINOTECAN LEUCOVORIN:27:11234:12:37:1.00:0.69:0.42:0.52), (CEFPIROME SULFATE:25:11236:19:30:1.00:0.57:0.45:0.51), (ETHIONAMIDE:24:11237:3:46:1.00:0.89: 0.34:0.49), (CEFOXITINCEFOXITIN SODIUM:22:11239:15:34:1.00:0.59:0.39:0.47), (GOLD:20:11241:27:22:1.00:0.43:0.48:0.45), (PITOFENONE:18:11243:16:33:1.00:0.53:0.35:0.42), (ABACAVIRLAMIVUDINE:17:112 44:4:45:1.00:0.81:0.27:0.41), (CARPRONIUM CHLORIDE:15:11246:8:41:1.00:0.65:0.27:0.38), (DESLORATADINEPSEUDOEPHEDRINE SULFATE:15:11246:6:43:1.00:0.71:0.26:0.38), (POLYCARBOPHIL:15:11246:4:45:1.00:0.7 9:0.25:0.38), (CYSTEINE:14:11247:10:39:1.00:0.58:0.26:0.36), (DIPHENHYDRAMINE HYDROCHLORIDEPHENYLEPHRINE HYDROCHLORIDE:14:11247:9:40:1.00:0.61:0.26:0.36), (ACETAMINOPHENCHLORPHE NIRAMINE MALEATEDEXTROMETHORPHAN HYDROBROMIDEPHENYLEPHRINE HYDROCHLORIDE OR ACETAMINOPHENDEXTROMETHORPHAN HYDROBROMIDEDOXYLAMINE SUCCINATEPHENYLEPHRINE HYDROCHLORIDE:11:11250:1:48:1.00:0.92:0.19:0.31), (PLANTAGO OVATA SEED\PLANTAGO SEED:11:11250:1:48:1.00:0.92:0.19:0.31), (COUMARIN:10:11251:6:43:1.00:0.63:0.19:0.2 9), (BAMBUTEROL HYDROCHLORIDE:9:11252:1:48:1.00:0.90:0.16:0.27), (BISMUTH SUBSALICYLATEGLYCERINPROPYLENE GLYCOL:9:11252:10:39:1.00:0.47:0.19:0.27), (SENNOSIDE B:9:11252:31:18:1.00:0.23:0.33:0.27), (TELITHROMYCIN:9:11252:4:45:1.00:0.69:0.17:0.27), (LISDEXAMFETAMINE:8:11253:5:44:1.00:0.62:0.15:0.25), (MAGNESIUM HYDROXIDEPARAFFIN:8:11253:1:48:1.00:0.89:0.14:0.25), (CALCIUM CHLORIDEDEXTROSEMAGNESIUM CHLORIDE\SODIUM CHLORIDE SOLUTION:7:11254:0:49:1.00:1.00:0.13:0.22), (ALOGLIPTIN BENZOATEMETFORMIN HYDROCHLORIDE:6:11255:0:49:1.00:1.00:0.11:0.20), (DOCONEXENTICOSAPENT:6:11 255:3:46:1.00:0.67:0.12:0.20), (GLYCERINMINERAL OILPARAFFIN:6:11255:4:45:1.00:0.60:0.12:0.20), (ARGININE HYDROCHLORIDE:5:11256:0:49:1.00:1.00:0.09:0.17), (DEXAMETHASONENEOMYCIN: 5:11256:3:46:1.00:0.63:0.10:0.17), (SONIDEGIB:5:11256:0:49:1.00:1.00:0.09:0.17), (CHY MOTRYPSIN:4:11257:1:48:1.00:0.80:0.08:0.14), (CRATAEGUS LAEVIGATA FRUIT:4:11257:4:45:1.00:0.50:0.08:0.14), (IBUPROFEN LYSINE:4:11257:1:48:1.00:0.80:0.08:0.14), (POTASSIUM BROMIDE:4:11257:11:38:1.00:0.27:0.10:0.14), (ASCORBIC ACIDHESPERIDIN METHYLCHALCONERUSCUS ACULEATUS ROOT:3:11258:1:48:1.00:0.75:0.06:0.11), (CELECOXIBIBUPROFENNAPROXENPLACE BO:3:11258:1:48:1.00:0.75:0.06:0.11), (TEDIZOLID PHOSPHATE:3:11258:1:48:1.00:0.75:0.06:0.11), (BROMOCRIPTINE:172:11088:0:50:1.00: 1.00:0.77:0.87), (CHLOROQUINE PHOSPHATE:46:11214:15:35:1.00:0.75:0.57:0.65), (ACETAMINOPHEN\CAFFEINECARI SOPRODOLDICLOFENAC SODIUM:36:11224:12:38:1.00:0.75:0.49:0.59), (SUGAMMADEX SODIUM:29:11231:8:42:1.00:0.78:0.41:0.54), (AMLODIPINE BESYLATEAZILSARTAN:23:11237:3:47:1.00:0.88:0.33:0.48), (STREPTOMYCIN SULFATE:17:11243:20:30:1.00:0.46:0.36:0.40), (AMITRIPTYLINECHLORDIAZEPOXID E:14:11246:3:47:1.00:0.82:0.23:0.36), (NITROFURANTOIN MONOHYDRATE:13:11247:1:49:1.00:0.93:0.21:0.34), (ELECTROLYTES NOSPOLYETHYLENE GLYCOL 3350:12:11248:5:45:1.00:0.71:0.21:0.32), (OCTOTIAMINE:12:11248:10:40:1.00:0.55:0.23: 0.32), (BETAMETHASONE VALERATEFUSIDIC ACID:11:11249:4:46:1.00:0.73:0.19:0.31), (DENOPAMINE:10:11250:7:43:1.00:0.59:0.19:0 0.29), (XYLITOL:10:11250:7:43:1.00:0.59:0.19:0.29), (CIMETIDINEFAMOTIDINERANITI DINE HYDROCHLORIDE:9:11251:3:47:1.00:0.75:0.16:0.26), (TEA TREE OIL:9:11251:2:48:1.00:0.82:0.16:0.26), (AMLODIPINE BESYLATEINDAPAMIDEPERINDOPRIL ARGININE:8:11252:1:49:1.00:0.89:0.14:0.24), (BUTYLSCOPOLAMINE BROMIDEMETAMIZOLE SODIUM:8:11252:2:48:1.00:0.80:0.14:0.24), (CINNARIZINEDIMENHYDRINATE:8:11252: 1:49:1.00:0.89:0.14:0.24), (ESTRADIOL BENZOATE:8:11252:43:7:1.00:0.16:0.53:0.24), (EPINEPHRINE HYDROCHLORIDE:6:11254:1:49:1.00:0.86:0.11:0.19), (LIGHT MINERAL OILMINERAL OIL:6:11254:2:48:1.00:0.75:0.11:0.19), (PAPAIN:6:11254:0:50:1.00:1.00:0.11:0.19), (ACE TAMINOPHENCHLORPHENIRAMINE MALEATEDEXTROMETHORPHAN HYDROBROMIDEPSEUDOEPHEDRINE HYDROCHLORIDE:5:11255:4:46:1.00:0.56:0.10:0.17), (CANNABIS SATIVA SEED OIL:5:11255:4:46:1.00:0.56:0.10:0.17), (THIOGUANINE ANHYDROUS:5:11255:4:46:1.00:0.56:0.10:0.17), (BACITRACIN ZINCNEOMYCINNEOMYCIN SULFATEPOLYMYXIN B SULFATE:4:11256:0:50:1.00:1.00:0.07:0.14), (POTASSIUM CARBONATE\POTASSIUM CITRATE:4:11256:1:49:1.00:0.80:0.08:0.14), (POTASSIUM SULFATE:4:11256:2:48:1.00:0.67:0.08:0.14), (TERLIPRESSIN:4:11256:1:49:1.00:0.80:0.0 8:0.14), (BUPIVACAINE HYDROCHLORIDEEPINEPHRINE BITARTRATE:3:11257:1:49:1.00:0.75:0.06:0.11), (CAFFEINE CITRATE:3:11257:1:49:1.00:0.75:0.06:0.11), (LINCOMYCIN:3:11257:5:45:1.00:0.38:0.06: 0.11), (TOREMIFENE CITRATE:21:11238:9:42:1.00:0.70:0.33:0.45), (SULFAMERAZINETRIMETHOPRIM:20:1 1239:15:36:1.00:0.57:0.36:0.44), (COBALAMIN:17:11242:7:44:1.00:0.71:0.28:0.40), (MEP ENZOLATE BROMIDE:17:11242:27:24:1.00:0.39:0.41:0.40), (GUAIAZULENE:15:11244:9:42:1.00:0.63: 0.26:0.37), (CHLORPHENESIN CARBAMATE:14:11245:1:50:1.00:0.93:0.22:0.35), (TRIMETHOPRIM SULFATE:13:11246:3:48:1.00:0.81:0.21:0.34), (CAMPHOR (SYNTHETIC)LEVOMENTHOLMENTHYL SALICYLATE OR CAMPHOR (SYNTHETIC)MENTHOLMENTHYL SALICYLATE OR MENTHOLMENTHYL SALICYLATE:10:11249:11:40:1.00:0.48:0.20:0.28), (SOMATREM:10:11249:13:38:1.00:0.4 3:0.21:0.28), (LACTIC ACID:8:11251:3:48:1.00:0.73:0.14:0.24), (ARNICA MONTANA FLOWER:6:11253:1:50:1.00:0.86:0.11:0.19), (BEDAQUILINE FUMARATE:5:11254:1:50:1.00:0.83:0.09:0.16), (FOTEMUSTINE:5:11254:0:51:1.00:1.00: 0.09:0.16), (POLIOMYELITIS VACCINE:5:11254:1:50:1.00:0.83:0.09:0.16), (CLEBOPRIDE MALATEDIMETHICONE:4:11255:1:50:1.00:0.80:0.07:0.14), (DIPHENHYDRAMINE HYDROCHLORIDEIBUPROFEN:4:11255:0:51:1.00:1.00:0.07:0.14), (RUPATADINE:4:11 255:0:51:1.00:1.00:0.07:0.14), (ETHYL CHLORIDE:3:11256:1:50:1.00:0.75:0.06:0.11), (SELUMETINIB:3:11256:0:51:1.00:1.00:0.06:0.11), (EMPAGLIFLOZINMETFORMIN HYDROCHLORIDE:51:11207:43:9:1.00:0.54:0.85:0.66), (ACETAMINOPHENCHLORZO XAZONE:29:11229:7:45:1.00:0.81:0.39:0.53), (ACETAMINOPHENAPRONALIDECAFFE INEPROPYPHENAZONE:22:11236:30:22:1.00:0.42:0.50:0.46), (ASCORBIC ACIDIRON:21:11237:4:48:1.00:0.84:0.30:0.45), (PEPPERMINT:20:11238:3:49:1.00:0.87:0 0.29:0.43), (PIRIBEDIL:19:11239:10:42:1.00:0.66:0.31:0.42), (DARIFENACIN:18:11240:5:4 7:1.00:0.78:0.28:0.41), (IODINE\POTASSIUM IODIDE:16:11242:16:36:1.00:0.50:0.31:0.38), (TETANUS TOXOID, REDUCED DIPHTHERIA TOXOID AND ACELLULAR PERTUSSIS VACCINE ADSORBED, TDAP:14:11244:1:51:1.00:0.93:0.22:0.35), (CANRENOIC ACID:11:11247:1:51:1.00:0.92:0.18:0.30), (NIAOULI OILQUININE BENZOATETHIAMINE HYDROCHLORIDE:11:11247:10:42:1.00:0.52:0.21:0.30), (OPIPRAMOL HYDROCHLORIDE:11:11247:0:52:1.00:1.00:0.17:0.30), (FEVERFEW:10:11248:4:48:1.00: 0.71:0.17:0.28), (LIXISENATIDE:10:11248:2:50:1.00:0.83:0.17:0.28), (ROYAL JELLY:10:11248:1:51:1.00:0.91:0.16:0.28), (ALISKIRENHYDROCHLOROTHIAZIDE:9:1 1249:14:38:1.00:0.39:0.19:0.26), (TENIPOSIDE:8:11250:13:39:1.00:0.38:0.17:0.24), (GUAR GUM:5:11253:0:52:1.00:1.00:0.09:0.16), (LOMERIZINE HYDROCHLORIDE:5:11253:2:50:1.00:0.71:0.09:0.16), (CEFCAPENE:4:11254:1:51:1.00:0.80:0.07:0.13), (CIMETROPIUM BROMIDE:4:11254:0:52:1.00:1.00:0.07:0.13), (DIDANOSINE:107:11150:3:50:1.00:0.97:0.68:0.80), (ANAGLIPTIN:44:11213:26:27:1.00:0.63:0.62:0.62), (BUTORPHANOL:28:11229: 9:44:1.00:0.76:0.39:0.51), (CIFENLINE SUCCINATE:25:11232:7:46:1.00:0.78:0.35:0.49), (LOVASTATINNIACIN:21:11236:7:46:1 0.00:0.75:0.31:0.44), (NILVADIPINE:21:11236:12:41:1.00:0.64:0.34:0.44), (CHLORDIAZE POXIDECLIDINIUM:16:11241:11:42:1.00:0.59:0.28:0.38), (AMLODIPINELOSARTAN:1 5:11242:9:44:1.00:0.63:0.25:0.36), (LEVOMILNACIPRAN:13:11244:3:50:1.00:0.81:0.21:0.33), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE HYDROCHLORIDE:12:11245:8:45:1.00:0.60:0.21:0.31), (CALCIUM PHOSPHATE:12:11245:12:41:1.00:0.50:0.23:0.31), (STRONTIUM:11:11246:9:44:1.00:0.55: 0.20:0.29), (TERIPARATIDE ACETATE:11:11246:1:52:1.00:0.92:0.17:0.29), (LIPASE:10:11247:5:48:1.00:0.67:0.17:0.2 7), (TIROFIBAN HYDROCHLORIDE:10:11247:5:48:1.00:0.67:0.17:0.27), (METHAMPHETAMINE HYDROCHLORIDE:9:11248:1:52:1.00:0.90:0.15:0.25), (SILVER NITRATE:9:11248:3:50:1.00:0.75:0.15:0.25), (THYMOL:9:11248:12:41:1.00:0.43:0.18:0.2 5), (ALPROSTADILPAPAVERINEPHENTOLAMINE:8:11249:4:49:1.00:0.67:0.14:0.23), (DOLASETRON:7:11250:4:49:1.00:0.64:0.13:0.21), (MECHLORETHAMINE:7:11250:5:48:1 0.00:0.58:0.13:0.21), (SERELAXIN:7:11250:1:52:1.00:0.88:0.12:0.21), (ACETAMINOPHE NDEXTROMETHORPHAN HYDROBROMIDEPHENYLEPHRINE HYDROCHLORIDE:6:11251:4:49:1.00:0.60:0.11:0.18), (ACETAMINOPHENCHLORPHE NIRAMINE MALEATEPHENYLPROPANOLAMINE:5:11252:0:53:1.00:1.00:0.09:0.16), (CALCIUM DOBESILATE:5:11252:2:51:1.00:0.71:0.09:0.16), (GRAMICIDINNEOMYCIN SULFATENYSTATINTRIAMCINOLONE ACETONIDE:5:11252:0:53:1.00:1.00:0.09:0.16), (AMLODIPINE MALEATEOLMESARTAN MEDOXOMIL:4:11253:1:52:1.00:0.80:0.07:0.13), (CODEINE PHOSPHATEGUAIFENESINPHENIRAMINE MALEATE:4:11253:1:52:1.00:0.80:0.07:0.13), (RIFAMYCIN:4:11253:0:53:1.00:1.00:0.07: 0.13), (TETRAHYDROZOLINE:4:11253:2:51:1.00:0.67:0.07:0.13), (FLUORESCEIN SODIUM:3:11254:1:52:1.00:0.75:0.05:0.10), (CYANOCOBALAMINFOLIC ACID:38:11218:0:54:1.00:1.00:0.41:0.58), (CLENBUTEROL HYDROCHLORIDE:34:11222:36:18:1.00:0.49:0.65:0.56), (DOCONEXENT:19:11237:1:53:1 0.00:0.95:0.26:0.41), (OMEGA-6 FATTY ACIDS:15:11241:17:37:1.00:0.47:0.29:0.36), (WHEY:12:11244:6:48:1.00:0.67:0.20:0.31), (BROMPHENIRAMINE MALEATEDEXTROMETHORPHAN HYDROBROMIDEPSEUDOEPHEDRINE HYDROCHLORIDE:11:11245:4:50:1.00:0.73:0.18:0.29), (RED YEAST:11:11245:8:46:1.00:0.58:0.19:0.29), (DEXTROSE\WATER:8:11248:0:54:1.00:1.00: 0.13:0.23), (ALLOPURINOL SODIUM:5:11251:0:54:1.00:1.00:0.08:0.16), (AMARANTHCHLOROFORMCITRIC ACID MONOHYDRATEPIMPINELLA ANISUM WHOLE:4:11252:0:54:1.00:1.00:0.07:0.13), (BUPARLISIB HYDROCHLORIDE:4:11252:0:54:1.00:1.00:0.07:0.13), (LEVOCABASTINE:4:11252:1:53:1 0.00:0.80:0.07:0.13), (ETHINYL ESTRADIOLFERROUS FUMARATENORETHINDRONE ACETATE:35:11220:9:46:1.00:0.80:0.43:0.56), (INSULIN ASPARTINSULIN DEGLUDEC:25:11230:31:24:1.00:0.45:0.51:0.48), (LARONIDASE:23:11232:11:44:1.00:0.6 8:0.34:0.46), (GUAIFENESINPHENYLEPHRINE HYDROCHLORIDE:14:11241:24:31:1.00:0.37:0.31:0.34), (BUPIVACAINEEPINEPHRINE: 13:11242:13:42:1.00:0.50:0.24:0.32), (BETAMETHASONE DIPROPIONATEGENTAMICIN SULFATE:10:11245:8:47:1.00:0.56:0.18:0.27), (NIFUROXAZIDE:9:11246:3:52:1.00:0.75:0 0.15:0.25), (TIROPRAMIDE:9:11246:3:52:1.00:0.75:0.15:0.25), (FENUGREEK SEED:7:11248:1:54:1.00:0.88:0.11:0.20), (HYDROXOCOBALAMIN ACETATE:7:11248:10:45:1.00:0.41:0.13:0.20), (GILTERITINIB:6:11249:5:50:1.00:0.55:0.1 1:0.18), (CEFTIBUTEN:4:11251:0:55:1.00:1.00:0.07:0.13), (SODIUM ACETATE:4:11251:0:55:1.00:1.00:0.07:0.13), (ISEPAMICIN:2:11253:2:53:1.00:0.50:0.04: 0.07), (EFTRENONACOG ALFA:36:11218:0:56:1.00:1.00:0.39:0.56), (ACLARUBICIN HYDROCHLORIDE:28:11226:16:40:1.00:0.64:0.41:0.50), (PEGINTERFERON ALFA-2A OR PEGINTERFERON ALFA-2B OR PEGINTERFERON BETA-1A OR PEGINTERFERON LAMBDA-1A:21:11233:15:41:1.00:0.58:0.34:0.43), (SULBACTAM:15:11239:3:53:1.00:0.83:0.22:0.3), (CYANOCOBALAMINFOLIC ACIDIRON:11:11243:4:52:1.00:0.73:0.17:0.28), (DIHYDROERGOCRISTINE MESYLATE:10:11244:2:54:1.00:0.83:0.16:0.26), (FLOMOXEF:10:11244:21:35:1.00:0.32:0 0.22:0.26), (ADUCANUMAB:8:11246:13:43:1.00:0.38:0.16:0.22), (SEROTONIN:8:11246:5:5 1:1.00:0.62:0.14:0.22), (ALANINEARGININEASPARTIC ACIDGLUTAMIC ACIDGLYCINEHISTIDINEISOLEUCINELEUCINELYSINE ACETATEMETHIONINEPHENYLALANINEPROLINESERINETHREONINETRYPTOP HANTYROSINEVALINE:7:11247:1:55:1.00:0.88:0.11:0.20), (OK-432:7:11247:23:33:1.00:0.23:0.18:0.20), (TETANUS TOXOID, REDUCED DIPHTHERIA TOXOID AND ACELLULAR PERTUSSIS VACCINE, ADSORBED ANTIGENS:7:11247:0:56:1.00:1.00:0.11:0.20), (ESCARGOT:6:11248:1:55:1.00:0.86:0.10: 0.18), (INDAPAMIDEPERINDOPRIL ARGININE:6:11248:0:56:1.00:1.00:0.10:0.18), (ENALAPRILAT:5:11249:0:56:1.00:1.00:0.08:0.15), (HOMATROPINEHOMATROPINE HYDROBROMIDE:5:11249:2:54:1.00:0.71:0.08:0.15), (LYNESTRENOL:5:11249:8:48:1.00: 0.38:0.09:0.15), (ALUMINUM HYDROXIDEMAGNESIUM HYDROXIDEMAGNESIUM TRISILICATE:4:11250:2:54:1.00:0.67:0.07:0.13), (ASPIRIN CALCIUM:4:11250:0:56:1.00:1.00:0.07:0.13), (MAGNESIUM CHLORIDE\POTASSIUM CHLORIDE\SODIUM ACETATE ANHYDROUS\SODIUM CHLORIDE\SODIUM GLUCONATE:4:11250:0:56:1.00:1.00:0.07:0.13), (FRUCTOSE:3:11251:1:55:1.00:0.75:0.0 5:0.10), (ETHOSUXIMIDE:44:11209:14:43:0.99:0.76:0.51:0.61), (GUANABENZ ACETATE:38:11215:30:27:0.99:0.56:0.58:0.57), (ALISKIREN HEMIFUMARATEHYDROCHLOROTHIAZIDE:32:11221:31:26:0.99:0.51:0.55:0.53), (OB ETICHOLIC ACID:16:11237:16:41:0.99:0.50:0.28:0.36), (ARTEMETHERLUMEFANTRINE:6:11247:3:5 4:0.99:0.67:0.10:0.17), (BUSERELIN:6:11247:1:56:0.99:0.86:0.10:0.17), (ACETAMINOPH ENDIHYDROCODEINE BITARTRATE:5:11248:0:57:0.99:1.00:0.08:0.15), (GANCICLOVIR SODIUM:4:11249:1:56:0.99:0.80:0.07:0.12), (FERROUS SULFATEGASTRIC MUCIN:3:11250:2:55:0.99:0.60:0.05:0.10), (ETIMICIN SULFATE:24:11228:22:36:0.99:0.52:0.40:0.45), (ACRIVASTINEPSEUDOEPHEDRINE HYDROCHLORIDE:19:11233:13:45:0.99:0.59:0.30:0.40), (LICORICE ROOT EXTRACTRHUBARB:19:11233:11:47:0.99:0.63:0.29:0.40), (FELODIPINEMETOPROLOL SUCCINATE:18:11234:10:48:0.99:0.64:0.27:0.38), (HEPATITIS A VIRUS STRAIN HM175 ANTIGEN (FORMALDEHYDE INACTIVATED):9:11243:1:57:0.99:0.90:0.14:0.24), (ARBEKACIN SULFATE:6:11246:0:58:0.99:1.00:0.09:0.17), (COLISTIN SULFATE:6:11246:3:55:0.99:0.67:0.10:0.17), (PARECOXIB SODIUM:6:11246:2:56:0.99:0.75:0.10:0.17), (SULTAMICILLIN:6:11246:1:57:0.99:0.86:0.: 0.17), (CITRIC ACID MONOHYDRATETRISODIUM CITRATE DIHYDRATE:5:11247:10:48:0.99:0.33:0.09:0.15), (GLUCOSAMINEGLUCOSAMINE SULFATE:5:11247:0:58:0.99:1.00:0.08:0.15), (FIG FRUIT OILSORBITOL:4:11248:1:57:0.99:0.80:0.07:0.12), (MINERAL OIL EMULSION:4:11248:1:57:0.99:0.80:0.07:0.12), (CEPHALEXIN MONOHYDRATE:3:11249:1:57:0.99:0.75:0.05:0.09), (CLAVULANATE POTASSIUMTICARCILLIN DISODIUM:3:11249:1:57:0.99:0.75:0.05:0.09), (LEVOLEUCOVORIN:3:11249:2:56:0.99:0.60:0.05:0.09), (EMTRICITABINETENOFOVIR:67:11184:11:48:0.99:0.86:0.58:0.69), (PH OSPHOCREATINE SODIUM:39:11212:6:53:0.99:0.87:0.42:0.57), (.ALPHA.-TOCOPHEROLVITAMINS:34:11217:21:38:0.99:0.62:0.47:0.54), (PIRENZEPINE:30:11221: 17:42:0.99:0.64:0.42:0.50), (COLESTILAN CHLORIDE:29:11222:21:38:0.99:0.58:0.43:0.50), (IXEKIZUMAB:26:11225:9:50:0.99:0.74: 0.34:0.47), (DIPHENHYDRAMINE HYDROCHLORIDEDOXYLAMINE SUCCINATE:25:11226:9:50:0.99:0.74:0.33:0.46), (ALFALFA:18:11233:18:41:0.99:0.50:0.3 1:0.38), (EPHEDRA SINICA STEMHERBALS:18:11233:12:47:0.99:0.60:0.28:0.38), (AMANTADINE SULFATE:16:11235:7:52:0.99:0.70:0.24:0.35), (DROSPIRENONE:14:11237:6:53:0.99:0.70: 0.21:0.32), (HYDROCODONEIBUPROFEN:14:11237:2:57:0.99:0.88:0.20:0.32), (PRALA TREXATE:8:11243:1:58:0.99:0.89:0.12:0.21), (BALSAM PERUBISMUTH HYDROXIDEBISMUTH SUBGALLATEBORIC ACIDZINC OXIDE:7:11244:3:56:0.99:0.70:0.11:0.19), (CEPHRADINE:6:11245:5:54:0.99:0.55:0.10:0.17), (OXICONAZOLE NITRATE:6:11245:4:55:0.99:0.60:0.10:0.17), (YTTRIUM Y-90:6:11245:4:55:0.99:0.60:0.10:0.17), (HEPTAMINOL HYDROCHLORIDE:5:11246:1:58:0.99:0.83:0.08:0.14), (TROMETHAMINE:5:11246:4:55:0 0.99:0.56:0.08:0.14), (AVIBACTAM SODIUMCEFTAZIDIME:3:11248:1:58:0.99:0.75:0.05:0.09), (PERAZINE:3:11248:7:52:0.9 9:0.30:0.05:0.09), (DIPHTHERIA AND TETANUS TOXOIDS AND ACELLULAR PERTUSSIS VACCINE ADSORBED:20:11230:8:52:0.99:0.71:0.28:0.40), (DIMETHYL SULFOXIDE:19:11231:30:30:0.99:0.39:0.39:0.39), (ALOSETRON HYDROCHLORIDE:18:11232:4:56:0.99:0.82:0.24:0.38), (PEPSIN:18:11232:12:48:0.99:0.6 0:0.27:0.38), (BORON:17:11233:20:40:0.99:0.46:0.30:0.36), (ANHYDROUS CITRIC ACIDMAGNESIUM OXIDE\SODIUM PICOSULFATE:16:11234:7:53:0.99:0.70:0.23:0.35), (0.38:0.35), (PENTOBARBITAL:16:11234:18:42:0.99:0.47:0.28:0.35), (ESTRAMUSTINE PHOSPHATE SODIUM:15:11235:1:59:0.99:0.94:0.20:0.33), (CAFFEINEERGOTAMINE TARTRATE:13:11237:6:54:0.99:0.68:0.19:0.30), (LUMACAFTOR:12:11238:56:4:0.99:0.18: 0.75:0.29), (ASCORBIC ACIDBIOTINCALCIUM PANTOTHENATEFERROUS ASPARTO GLYCINATEFERROUS FUMARATEFOLIC ACIDNIACINPYRIDOXINE HYDROCHLORIDE\RIBOFLAVINTHIAMINE MONONITRATE:7:11243:1:59:0.99:0.88:0.11:0.19), (TROLAMINE:7:11243:6:54:0.99:0.54: 0.11:0.19), (CHLORHEXIDINE HYDROCHLORIDENEOMYCIN SULFATE:5:11245:2:58:0.99:0.71:0.08:0.14), (GLUCOSE, LIQUIDPOTASSIUM CHLORIDE\SODIUM CHLORIDE:5:11245:0:60:0.99:1.00:0.08:0.14), (NITROUS OXIDEOXYGEN:4:11246:0:60:0.99:1.00:0.06:0.12), (ARTESUNATE:3:11247:3:57:0.99:0.50:0.05:0.09), (ISOPROPYL ALCOHOLLEUPROLIDE ACETATENORETHINDRONE ACETATE:58:11191:27:34:0.99:0.68:0.63:0.66), (DIENOGEST:28:11221:29:32:0.99:0.49:0 0.47:0.48), (EFLORNITHINE HYDROCHLORIDE:27:11222:6:55:0.99:0.82:0.33:0.47), (ESTROGENS, CONJUGATED SYNTHETIC B:26:11223:3:58:0.99:0.90:0.31:0.46), (CETRAXATE HYDROCHLORIDE:20:11229:3:58:0.99:0.87:0.26:0.40), (HYDROXOCOBALAMINPYRI DOXINE HYDROCHLORIDETHIAMINE HYDROCHLORIDE:20:11229:8:53:0.99:0.71:0.27:0.40), (FOSINOPRIL SODIUMHYDROCHLOROTHIAZIDE:19:11230:7:54:0.99:0.73:0.26:0.38), (CARBINOXA MINE MALEATE:15:11234:3:58:0.99:0.83:0.21:0.33), (HYPERICUM PERFORATUM:13:11236:14:47:0.99:0.48:0.22:0.30), (BEXAROTENE:11:11238:14:47:0.99: 0.44:0.19:0.27), (LIDOCAINETRIBENOSIDE:9:11240:4:57:0.99:0.69:0.14:0.23), (LACT OBACILLUS CASEI:8:11241:1:60:0.99:0.89:0.12:0.21), (DELAMANID:7:11242:0:61:0.99:1.00:0.10:0.1 9), (ASPARTIC ACID:5:11244:3:58:0.99:0.63:0.08:0.14), (PHENDIMETRAZINE TARTRATE:5:11244:0:61:0.99:1.00:0.08:0.14), (TRICLOFOS SODIUM:5:11244:0:61:0.99:1.00:0.08:0.14), (ONARTUZUMAB:4:11245:0:61:0.99:1.00:0.06:0.12), (PHOLCODINE:4:11245:0:61:0.99:1.00:0.06:0.12), (WITCH HAZEL:4:11245:0:61:0.99:1.00:0.06:0.12), (BISMUTH SUBSALICYLATEMETRONIDAZOLETETRACYCLINE HYDROCHLORIDE:3:11246:2:59:0.99:0.60:0.05:0.09), (DALBAVANCIN:3:11246:1:60:0.9 9:0.75:0.05:0.09), (EQUINE BOTULINUM NEUROTOXIN A/B/C/D/E/F/G IMMUNE FAB2:3:11246:1:60:0.99:0.75:0.05:0.09), (PYRITHIONE ZINC:3:11246:0:61:0.99:1.00:0.05:0.09), (SULTAMICILLIN TOSYLATE:3:11246:1:60:0.99:0.75:0.05:0.09), (SILICON DIOXIDE:43:11205:26:36:0.99:0.62:0.54:0.58), (ASPIRINBUTALBITALCAFFEINECOD EINE PHOSPHATE:25:11223:6:56:0.99:0.81:0.31:0.45), (PHENELZINE:25:11223:14:48:0.99:0.64: 0.34:0.45), (GARENOXACIN:22:11226:16:46:0.99:0.58:0.32:0.42), (PALIFERMIN:19:112 29:3:59:0.99:0.86:0.24:0.38), (DYDROGESTERONEESTRADIOL:18:11230:7:55:0.99:0.72: 0.25:0.37), (HEMP:18:11230:6:56:0.99:0.75:0.24:0.37), (SODIUM PHOSPHATE, DIBASIC, ANHYDROUS\SODIUM PHOSPHATE, DIBASIC, HEPTAHYDRATE\SODIUM PHOSPHATE, MONOBASIC, MONOHYDRATE:14:11234:33:29:0.99:0.30:0.33:0.31), (BENZALKONIUM CHLORIDE:11:11237:6:56:0.99:0.65:0.16:0.26), (ACETAMINOPHEN\CAFFEINECODEINE PHOSPHATE:7:11241:4:58:0.99:0.64:0.11:0.18), (CAPREOMYCIN:4:11244:2:60:0.99:0.67: 0.06:0.11), (CHLOROPYRAMINE:4:11244:0:62:0.99:1.00:0.06:0.11), (CHLORPHENIRA MINE MALEATEIBUPROFENPHENYLEPHRINE HYDROCHLORIDE:4:11244:1:61:0.99:0.80:0.06:0.11), (PROTEIN HYDROLYSATE:4:11244:0:62:0.99:1.00:0.06:0.11), (LOTEPREDNOL:2:11246:0:62:0.99:1 0.00:0.03:0.06), (DOXYLAMINE SUCCINATEPYRIDOXINE HYDROCHLORIDE:40:11207:18:45:0.99:0.69:0.47:0.56), (SARPOGRELATE HYDROCHLORIDE:33:11214:38:25:0.99:0.46:0.57:0.51), (FERRIC POLYSACCHARIDE COMPLEX:29:11218:27:36:0.99:0.52:0.45:0.48), (HYDROCHLOROTHIAZIDEMETOPRO LOL TARTRATE:25:11222:12:51:0.99:0.68:0.33:0.44), (FOSFOMYCIN CALCIUM:24:11223:25:38:0.99:0.49:0.39:0.43), (GRAZOPREVIR:24:11223:10:53:0.99:0.7 1:0.31:0.43), (ROXATIDINE ACETATE HYDROCHLORIDE:20:11227:9:54:0.99:0.69:0.27:0.39), (CANNABIS SATIVA SUBSP. SATIVA FLOWERING TOP:19:11228:9:54:0.99:0.68:0.26:0.38), (SODIUM PHOSPHATE, DIBASIC:19:11228:9:54:0.99:0.68:0.26:0.38), (TIARAMIDE:19:11228:13:50:0.99:0.59:0.28: 0.38), (BACITRACINNEOMYCIN SULFATE:18:11229:10:53:0.99:0.64:0.25:0.36), (ACAMPROSATE:17:11230:27:36:0.99:0.3 9:0.32:0.35), (AMINOBENZOIC ACIDCALCIUM PANTOTHENATECYSTINEKERATINTHIAMINE MONONITRATE:16:11231:11:52:0.99:0.59:0.24:0.34), (CAMPHORCAPSICUMMETHYL SALICYLATE:16:11231:8:55:0.99:0.67:0.23:0.34), (BETAMETHASONE DIPROPIONATECALCIPOTRIENE HYDRATE:13:11234:39:24:0.99:0.25:0.35:0.29), (LINSEED OIL:13:11234:3:60:0.99:0.81:0.18:0.29), (HALOPERIDOL LACTATE:12:11235:1:62:0.99:0.92:0.16:0.28), (METHYLTESTOSTERONE:10:11237:0:63: 0.99:1.00:0.14:0.24), (CHLORHEXIDINE GLUCONATEISOPROPYL ALCOHOL:9:11238:0:63:0.99:1.00:0.13:0.22), (PILSICAINIDE:8:11239:11:52:0.99:0.42:0.13:0.20), (DOMPERIDONE MALEATE:7:11240:0:63:0.99:1.00:0.10:0.18), (HOMOSALATEOCTINOXATEOCTISAL ATEOXYBENZONETITANIUM DIOXIDE:5:11242:0:63:0.99:1.00:0.07:0.14), (RUCAPARIB:4:11243:0:63:0.99:1.00:0.06:0 0.11), (TRICLOSAN:4:11243:5:58:0.99:0.44:0.06:0.11), (TESTOSTERONE DECANOATETESTOSTERONE ISOCAPROATETESTOSTERONE PHENYLPROPIONATETESTOSTERONE PROPIONATE:3:11244:1:62:0.99:0.75:0.05:0.09), (HUMAN IMMUNOGLOBULIN GHYALURONIDASE RECOMBINANT HUMAN:129:11117:2:62:0.99:0.98:0.68:0.80), (ABACAVIR SULFATE:118:11128:1:63:0.99:0.99:0.65:0.79), (FLUOROURACILLEUCOVORIN CALCIUMOXALIPLATIN:56:11190:23:41:0.99:0.71:0.58:0.64), (LOPINAVIR:39:11207:8: 56:0.99:0.83:0.41:0.55), (COBICISTATELVITEGRAVIREMTRICITABINETENOFOVIR: 34:11212:5:59:0.99:0.87:0.37:0.52), (SAPROPTERIN DIHYDROCHLORIDE:32:11214:20:44:0.99:0.62:0.42:0.50), (TOFISOPAM:31:11215:11:53: 0.99:0.74:0.37:0.49), (ACETAMINOPHEN\CAFFEINECARISOPRODOLDICLOFENAC:2 9:11217:11:53:0.99:0.73:0.35:0.48), (ELBASVIR:25:11221:10:54:0.99:0.71:0.32:0.44), (LE VOBUNOLOL HYDROCHLORIDE:20:11226:10:54:0.99:0.67:0.27:0.38), (LURASIDONE:19:11227:7:57:0.99:0.73:0.25:0.37), (PIRARUBICIN HYDROCHLORIDE:17:11229:5:59:0.99:0.77:0.22:0.35), (PROTRIPTYLINE:16:11230:2:62: 0.99:0.89:0.21:0.33), (INVERT SUGAR:11:11235:1:63:0.99:0.92:0.15:0.26), (THIETHYLPERAZINE MALATE:9:11237:6:58:0.99:0.60:0.13:0.22), (FOLIC ACIDIRON:8:11238:4:60:0.99:0.67:0.12:0.20), (PRIMAQUINE PHOSPHATE:8:11238:0:64:0.99:1.00:0.11:0.20), (SILVER:8:11238:2:62:0.99:0.80:0.11:0.2 0), (FERROUS SULFATEPROTEASE:6:11240:1:63:0.99:0.86:0.09:0.16), (NEOSTIGMINE BROMIDE:6:11240:7:57:0.99:0.46:0.10:0.16), (NORADRENALINE HYDROCHLORIDE:6:11240:0:64:0.99:1.00:0.09:0.16), (PENTOXYVERINE:6:11240:3:61:0 0.99:0.67:0.09:0.16), (VARENICLINE:6:11240:1:63:0.99:0.86:0.09:0.16), (IPATASERTIB:5: 11241:1:63:0.99:0.83:0.07:0.14), (CEFMINOX SODIUM:4:11242:1:63:0.99:0.80:0.06:0.11), (DIMETHINDENE MALEATE:4:11242:0:64:0.99:1.00:0.06:0.11), (NEDOCROMIL SODIUM:3:11243:1:63:0.99:0.75:0.05:0.09), (SORAFENIB:606:10639:0:65:0.99:1.00:0.90: 0.95), (CYPROTERONE ACETATEETHINYL ESTRADIOL:51:11194:11:54:0.99:0.82:0.49:0.61), (ASCORBIC ACID/CHROMIUM/SELENIUM/VANADYL SULFATE:30:11215:34:31:0.99:0.47:0.49:0.48), (PENTAERYTHRITOL TETRANITRATE:25:11220:10:55:0.99:0.71:0.31:0.43), (TRANYLCYPROMINE SULFATE:25:11220:14:51:0.99:0.64:0.33:0.43), (CALCIUM ACETATEMAGNESIUM ACETATE\POTASSIUM ACETATE\SODIUM ACETATE\SODIUM CHLORIDE:22:11223:9:56:0.99:0.71:0.28:0.40), (IXABEPILONE:22:11223:13:52:0.99:0.63: 0.30:0.40), (AROTINOLOL:20:11225:10:55:0.99:0.67:0.27:0.38), (RUPATADINE FUMARATE:18:11227:26:39:0.99:0.41:0.32:0.36), (CYPROTERONE:17:11228:19:46:0.99: 0.47:0.27:0.34), (BORIC ACID:15:11230:14:51:0.99:0.52:0.23:0.32), (AMINOPHYLLINEQUININE SULFATE:12:11233:3:62:0.99:0.80:0.16:0.27), (ALGINIC ACID:10:11235:2:63:0.99:0.83:0.14:0.24), (ALPROSTADIL ALFADEX:9:11236:3:62:0.99:0.75:0.13:0.22), (CASTOR OIL:9:11236:1:64:0.99:0.90:0.12:0.22), (HESPERIDIN:9:11236:2:63:0.99:0.82:0.13:0.22), (SODIUM HYPOCHLORITE:9:11236:9:56:0.99:0.50:0.14:0.22), (OXANDROLONE:8:11237:15:50:0.9 9:0.35:0.14:0.20), (TROLAMINE SALICYLATE:7:11238:1:64:0.99:0.88:0.10:0.18), (ABACAVIR SULFATELAMIVUDINEZIDOVUDINE:5:11240:0:65:0.99:1.00:0.07:0.13), (LEUCOVORIN SODIUM:5:11240:0:65:0.99:1.00:0.07:0.13), (NORDAZEPAM:5:11240:0:65:0.99:1.00:0.07: 0.13), (KETOBEMIDONE HYDROCHLORIDE:4:11241:2:63:0.99:0.67:0.06:0.11), (METHOXSALEN:4:11241:1:64:0.9 9:0.80:0.06:0.11), (SODIUM THIOSULFATE:4:11241:0:65:0.99:1.00:0.06:0.11), (PENICILLIN G SODIUM:3:11242:1:64:0.99:0.75:0.04:0.08), (PROCARBAZINE:90:11154:4:62:0.99:0.96:0.59:0.73), (DIETARY SUPPLEMENTRESVERATROL:30:11214:12:54:0.99:0.71:0.36:0.48), (LEVOSIMENDAN: 23:11221:34:32:0.99:0.40:0.42:0.41), (TENOXICAM:23:11221:10:56:0.99:0.70:0.29:0.41), (ABCIXIMAB:15:11229:1:65:0.99:0.94:0.19:0.31), (FLOPROPIONE:14:11230:8:58:0.99:0.6 4:0.19:0.30), (TIAPROFENIC ACID:14:11230:7:59:0.99:0.67:0.19:0.30), (TAZOBACTAM SODIUM:13:11231:8:58:0.99:0.62:0.18:0.28), (NATTOKINASE:11:11233:3:63:0.99:0.79:0.: 0.25), (POTASSIUM CHLORIDE\SODIUM CHLORIDE:11:11233:7:59:0.99:0.61:0.16:0.25), (SIBUTRAMINE:10:11234:1:65:0.99:0.91: 0.13:0.23), (BRIGATINIB:8:11236:1:65:0.99:0.89:0.11:0.20), (CAMPHOR:7:11237:0:66:0.9 9:1.00:0.10:0.18), (ACETAMINOPHENDEXTROMETHORPHAN HYDROBROMIDEGUAIFENESINPHENYLEPHRINE HYDROCHLORIDE:5:11239:1:65:0.99:0.83:0.07:0.13), (AMINO ACIDSCARBOHYDRATESELECTROLYTES NOS:4:11240:0:66:0.99:1.00:0.06:0.11), (SODIUM IODIDE I-131:4:11240:0:66:0.99:1.00:0.06:0.11), (POLYMYXIN B SULFATETRIMETHOPRIM SULFATE:41:11202:28:39:0.99:0.59:0.51:0.55), (ZUCLOPENTHIXOL:39:11204:6:61:0.99: 0.87:0.39:0.54), (TREPROSTINIL SODIUM:25:11218:6:61:0.99:0.81:0.29:0.43), (ACIDOPHILUSLACTOBACILLUS RHAMNOSUS:18:11225:4:63:0.99:0.82:0.22:0.35), (BISACODYL OR GLYCERIN OR MINERAL OIL OR SODIUM PHOSPHATE, DIBASIC\SODIUM PHOSPHATE, MONOBASIC:17:11226:3:64:0.99:0.85:0.21:0.34), (METHSCOPOLAMINE BROMIDE:17:11226:0:67:0.99:1.00:0.20:0.34), (MIPOMERSEN SODIUM:17:11226:3:64:0.99:0.85:0.21:0.34), (PREDNISOLONE ACETATESULFACETAMIDE SODIUM:17:11226:24:43:0.99:0.41:0.28:0.34), (ACOTIAMIDE HYDROCHLORIDE:12:11231:8:59:0.99:0.60:0.17:0.26), (MEFLOQUINE:8:11235:1:66:0.99: 0.89:0.11:0.19), (POTASSIUM PHOSPHATE, DIBASIC\POTASSIUM PHOSPHATE, MONOBASIC\SODIUM PHOSPHATE, DIBASIC\SODIUM PHOSPHATE, MONOBASIC:7:11236:1:66:0.99:0.88:0.10:0.17), (ARABICA COFFEE BEANCOFFEE BEAN:6:11237:2:65:0.99:0.75:0.08:0.15), (POLIDOCANOL:6:11237:5:62:0.99:0.55:0.09:0. 15), (MACITENTAN:329:10913:1:67:0.99:1.00:0.83:0.91), (STAVUDINE:123:11119:2:66:0.99:0.98:0.65:0.78), (IPRAGLIFLOZIN L-PROLINE:39:11203:20:48:0.99:0.66:0.45:0.53), (ESTRADIOL HEMIHYDRATE:35:11207:21:47:0.99:0.63:0.43:0.51), (MAPROTILINE HYDROCHLORIDE:32:11210:18:50:0.99:0.64:0.39:0.48), (ETHINYL ESTRADIOLETHYNODIOL DIACETATE:31:11211:8:60:0.99:0.79:0.34:0.48), (ESTRADIOL ACETATE:30:11212:15:53:0.99:0.67:0.36:0.47), (DESLANOSIDE:22:11220:6:62:0.99:0.79: 0.26:0.39), (.BETA.-ACETYLDIGOXIN:20:11222:8:60:0.99:0.71:0.25:0.37), (DIFLUCORTOLONE VALERATELIDOCAINE:17:11225:16:52:0.99:0.52:0.25:0.33), (BENZOIC ACIDHYOSCYAMINE SULFATEMETHENAMINEMETHYLENE BLUEPHENYL SALICYLATE:16:11226:9:59:0.99:0.64:0.21:0.32), (CYANOCOBALAMIN CO-57LEVOMEFOLATE CALCIUMPYRIDOXINE HYDROCHLORIDE\RIBOFLAVIN:15:11227:1:67:0.99:0.94:0.18:0.31), (DIHYDRALAZINE SULFATE:15:11227:4:64:0.99:0.79:0.19:0.31), (ALDIOXA:11:11231:6:62:0.99:0.65:0.15:0 0.24), (BETAMETHASONE ACETATEBETAMETHASONE SODIUM PHOSPHATE:10:11232:10:58:0.99:0.50:0.15:0.23), (DEXTROSE MONOHYDRATE\SODIUM CHLORIDE:10:11232:7:61:0.99:0.59:0.14:0.23), (PRANOPROFEN:10:11232:3:65:0.99:0.77: 0.13:0.23), (IOBITRIDOL:7:11235:2:66:0.99:0.78:0.10:0.17), (CELLULOSE, OXIDIZED:5:11237:1:67:0.99:0.83:0.07:0.13), (IBUPROFENPHENYLEPHRINE HYDROCHLORIDE:5:11237:1:67:0.99:0.83:0.07:0.13), (AMINO ACIDSANHYDROUS DEXTROSECALCIUM CHLORIDEMAGNESIUM SULFATE HEPTAHYDRATE\POTASSIUM CHLORIDE\SODIUM ACETATE\SODIUM GLYCEROPHOSPHATE ANHYDROUSSOYBEAN OIL:4:11238:0:68:0.99:1.00:0.06:0.11), (DEXAMETHASONE PHOSPHATE:4:11238:0:68:0.99:1.00:0.06:0.11), (MAGNESIUM CHLORIDE\POTASSIUM CHLORIDE\SODIUM ACETATE\SODIUM CHLORIDE\SODIUM GLUCONATE:4:11238:1:67:0.99:0.80:0.06:0.11), (OXIRACETAM:4:11238:0:68:0.99:1.00: 0.06:0.11), (TIXOCORTOL PIVALATE:4:11238:0:68:0.99:1.00:0.06:0.11), (EPINEPHRINELIDOCAINE HYDROCHLORIDE:3:11239:1:67:0.99:0.75:0.04:0.08), (INSULIN DEFALAN (PORCINE):3:11239:1:67:0.99:0.75:0.04:0.08), (EFAVIRENZ:294:10947:1:68:0.99:1.00:0.8 1:0.89), (FLUPIRTINE:27:11214:4:65:0.99:0.87:0.29:0.44), (LEVOBUNOLOL:23:11218:9:6 0:0.99:0.72:0.28:0.40), (BACITRACIN ZINC:22:11219:16:53:0.99:0.58:0.29:0.39), (FURSULTIAMINE:21:11220:55:14:0.99:0.28:0 0.60:0.38), (BETAMETHASONE SODIUM PHOSPHATENEOMYCIN SULFATE:17:11224:33:36:0.99:0.34:0.32:0.33), (ORYCTOLAGUS CUNICULUS SKIN:16:11225:1:68:0.99:0.94:0.19:0.32), (COAL TAR:10:11231:1:68:0.99:0.91:0.13:0.22), (DEMECLOCYCLINE:10:11231:13:56:0.99:0.43: 0.15:0.22), (HYDROCHLOROTHIAZIDEMETOPROLOL:8:11233:4:65:0.99:0.67:0.11:0.1 9), (PROCAINAMIDE HYDROCHLORIDE:8:11233:1:68:0.99:0.89:0.11:0.19), (GLYCOPYRRONIUMFORMOTE ROL FUMARATE:6:11235:4:65:0.99:0.60:0.08:0.15), (ALOE:4:11237:1:68:0.99:0.80:0.06:0.10), (LIDOCAINETETRACAINE:4:11237:0:69:0.99:1.00:0.05:0.10), (ALUMINUM PHOSPHATE:3:11238:1:68:0.99:0.75:0.04:0.08), (ASPIRIN®XYCODONE HYDROCHLORIDE:3:11238:1:68:0.99:0.75:0.04:0.08), (METHOTREXATE SODIUM:1206:10034:0:70:0.99:1.00:0.95:0.97), (CLOPERASTINE:38:11202:20:50:0.99:0.6 6:0.43:0.52), (PRAMOXINE HYDROCHLORIDE:37:11203:31:39:0.99:0.54:0.49:0.51), (COBICISTATDARUNAVIR ETHANOLATE:36:11204:8:62:0.99:0.82:0.37:0.51), (NAPHAZOLINE HYDROCHLORIDE:34:11206:6:64:0.99:0.85:0.35:0.49), (SULFACETAMIDE SODIUMSULFUR:33:11207:18:52:0.99:0.65:0.39:0.49), (ECABET SODIUM:31:11209:25:45:0.99:0.55:0.41:0.47), (COUMARINTROXERUTIN:27:11213:5:65: 0.99:0.84:0.29:0.44), (PHENYLEPHRINE HYDROCHLORIDETROPICAMIDE:27:11213:5:65:0.99:0.84:0.29:0.44), (1217:14:56:0.99:0.62:0.29:0.40), (BENPROPERINE PHOSPHATE:22:11218:13:57:0.99:0.63:0.28:0.39), (CITRIC ACID, 1-STEARYL ESTERSODIUM CITRATE:21:11219:1:69:0.99:0.95:0.23:0.38), (OMACETAXINE MEPESUCCINATE:20:11220:2:68:0.99:0.91:0.23:0.36), (FUSIDATE SODIUM:15:11225:15:55:0.99:0.50:0.21:0.30), (METYRAPONE:15:11225:13:57:0.99:0.54: 0.21:0.30), (CALCIUMCHOLECALCIFEROLCOPPERMAGNESIUMMANGANESEZIN C:11:11229:3:67:0.99:0.79:0.14:0.24), (NAFAMOSTAT:7:11233:5:65:0.99:0.58:0.10:0.17), (PRAMOXINE:7:11233:1:69:0.99:0.88:0.09:0.17), (GRISEOFULVIN:6:11234:2:68:0.99:0.7 5:0.08:0.15), (IDASANUTLIN:4:11236:0:70:0.99:1.00:0.05:0.10), (PEGLOTICASE:4:11236: 0:70:0.99:1.00:0.05:0.10), (SODIUM FEREDETATE:4:11236:1:69:0.99:0.80:0.05:0.10), (SULFUR HEXAFLUORIDE:4:11236:0:70:0.99:1.00:0.05:0.10), (DEXTROSE\SODIUM CHLORIDE:3:11237:1:69:0.99:0.75:0.04:0.08), (INOSINE:3:11237:1:69:0.99:0.75:0.04:0.0 8), (BUSULFAN:402:10837:0:71:0.99:1.00:0.85:0.92), (CHIMAPHILA UMBELLATAMANGANESE CHLORIDEPOPULUS TREMULOIDESSODIUM TAUROCHOLATE:35:11204:16:55:0.99:0.69:0.39:0.50), (CALCIUM CARBONATEETIDRONATE DISODIUM:27:11212:25:46:0.99:0.52:0.37:0.43), (ERYTHROMYCINSULFISOXAZOLE:2 5:11214:21:50:0.99:0.54:0.33:0.41), (FERROUS CITRATE\SODIUM:23:11216:3:68:0.99:0.88:0.25:0.39), (GLIMEPIRIDEROSIGLITAZONE MALEATE:23:11216:53:18:0.99:0.30:0.56:0.39), (TOPIROXOSTAT:20:11219:11:60:0.99:0.65:0.25:0.36), (AMDINOCILLIN PIVOXIL HYDROCHLORIDE:19:11220:3:68:0.99:0.86:0.22:0.35), (FERRIC SODIUM CITRATE:19:11220:7:64:0.99:0.73:0.23:0.35), (METHAZOLAMIDE:11:11228:3:68:0.99:0.79:0.14:0.24), (MENINGOCOCCAL POLYSACCHARIDE VACCINE AMENINGITIDIS GROUP A CAPSULAR POLYSACCHARIDE ANTIGEN:9:11230:2:69:0.99:0.82:0.12:0.20), (INULIN:8:11231:3:68:0.99:0.73:0.11:0.18), (TOBRAMYCIN SULFATE:8:11231:6:65:0.99:0.57:0.11:0.18), (SULFUR:7:11232:1:70:0.99:0.88:0.09:0.16), (GELATIN\SODIUM CHLORIDE:4:11235:0:71:0.99:1.00:0.05:0.10), (OXOMEMAZINE:4:11235:0:71:0.99:1.00: 0.05:0.10), (BENOXINATE HYDROCHLORIDE:3:11236:1:70:0.99:0.75:0.04:0.08), (PHENYLPROPANOLAMINE:3:11 236:4:67:0.99:0.43:0.04:0.08), (FACTOR XIII CONCENTRATE (HUMAN):2:11237:0:71:0.99:1.00:0.03:0.05), (ASUNAPREVIR:103:11135:36:36:0.99:0.74: 0.74:0.74), (GADOBENATE DIMEGLUMINE:59:11179:13:59:0.99:0.82:0.50:0.62), (HYDRAZINE SULFATE:31:11207:11:61:0.99:0.74:0.34:0.46), (HYDROQUINONE:25:11213:15:57:0.99:0 0.63:0.30:0.41), (GREEN TEA LEAF:19:11219:15:57:0.99:0.56:0.25:0.35), (TYROSINE:19:11219:2:70:0.99:0.90:0.21:0.3), (CALCIUM PANTOTHENATE:18:11220:2:70:0.99:0.90:0.20:0.33), (MOGAMULIZUMAB:14:11224:19: 53:0.99:0.42:0.21:0.28), (DEZOCINE:13:11225:0:72:0.99:1.00:0.15:0.27), (BENZETHONIUM CHLORIDE:11:11227:4:68:0.99:0.73:0.14:0.23), (MEPROBAMATE:9:11229:1:71:0.99:0.90: 0.11:0.20), (DONEPEZIL HYDROCHLORIDEMEMANTINE HYDROCHLORIDE:7:11231:2:70:0.99:0.78:0.09:0.16), (TAVABOROLE:7:11231:0:72:0.99: 1.00:0.09:0.16), (ETHINYL ESTRADIOLNORELGESTROMIN:137:11100:1:72:0.99:0.99:0.66:0.79), (ALGLUCOSIDASE ALFA:76:11161:19:54:0.99:0.80:0.58:0.68), (BRIVARACETAM:52:11185:30:43:0.99:0.63: 0.55:0.59), (FERRIC HYDROXIDE:43:11194:49:24:0.99:0.47:0.64:0.54), (FLUPENTIXOL:34:11203:9:64:0.99:0.79:0.35:0.48), (VONOPRAZAN:33:11204:24:49:0.99:0.58:0.40:0.47), (ALBUTEROLIPRA TROPIUM BROMIDE:31:11206:30:43:0.99:0.51:0.42:0.46), (INFLUENZA VIRUS VACCINE TRIVALENT TYPE A+B AFLURIA:26:11211:7:66:0.99:0.79:0.28:0.42), (EPROSARTAN MESYLATE:22:11215:1:72:0.99:0.96:0.23:0.38), (TRIFLUOPERAZINETRIFLUOPERAZINE HYDROCHLORIDE:22:11215:5:68:0.99:0.81:0.24:0.38), (ACEMETACIN:21:11216:3:70:0.9 9:0.88:0.23:0.37), (VELAGLUCERASE ALFA:21:11216:14:59:0.99:0.60:0.26:0.37), (DUPILUMAB:18:11219:5:68:0.99:0.78:0.21:0 0.33), (MANGANESEMANGANESE CHLORIDE:16:11221:8:65:0.99:0.67:0.20:0.30), (ALECTINIB HYDROCHLORIDE:12:11225:2:71:0.99:0.86:0.14:0.25), (AMLODIPINEHYDROCHLORO THIAZIDEOLMESARTAN:8:11229:1:72:0.99:0.89:0.10:0.18), (ASPIRINGLYCINE:4:112 33:0:73:0.99:1.00:0.05:0.10), (CAMPHOR (SYNTHETIC)MENTHOL:4:11233:0:73:0.99:1.00:0.05:0.10), (VINDESINE SULFATE:35:11201:26:48:0.99:0.57:0.42:0.49), (MILRINONE LACTATE:25:11211:18:56:0.99:0.58:0.31:0.40), (LEVOMEFOLIC ACID:22:11214:2:72:0.99:0.92:0.23:0.37), (ISAVUCONAZOLE:21:11215:8:66:0.99:0.72:0.24:0.36), (BAZEDOXIFENE:18:11218:1:73:0.99:0.95:0.20:0.33), (.GAMMA.-AMINOBUTYRIC ACID:17:11219:4:70:0.99:0.81:0.20:0.31), (RILPIVIRINE:15:11221:11:63:0.99:0.58:0.19:0.29), (DESIPRAMINE HYDROCHLORIDE:13:11223:0:74:0.99:1.00:0.15:0.26), (LEVAMLODIPINE:10:11226:12: 62:0.99:0.45:0.14:0.21), (NALOXEGOL:8:11228:1:73:0.99:0.89:0.10:0.18), (GONADORE LIN:4:11232:0:74:0.99:1.00:0.05:0.10), (PROPOXYPHENE HYDROCHLORIDE:4:11232:1:73:0.99:0.80:0.05:0.10), (EMTRICITABINETENOFOVIR DISOPROXIL:201:11034:1:74:0.99:1.00:0.73:0.84), (IVACAFTORLUMACAFTOR:115:11 120:7:68:0.99:0.94:0.63:0.75), (EMTRICITABINERILPIVIRINETENOFOVIR DISOPROXIL FUMARATE:74:11161:22:53:0.99:0.77:0.58:0.66), (AZITHROMYCIN MONOHYDRATE:52:11183:23:52:0.99:0.69:0.50:0.58), (CARBOXYMETHYLCELLULOSE SODIUMGLYCERINPOLYSORBATE 80:43:11192:12:63:0.99:0.78:0.41:0.53), (APRINDINE HYDROCHLORIDE:40:11195:13:62:0.99:0.75:0.39:0.52), (TROXIPIDE:40:11195:17:58:0.9 9:0.70:0.41:0.52), (ACETAMINOPHENMETHOCARBAMOL:35:11200:34:41:0.99:0.51:0.46:0.48), (RANIMUSTINE:28:11207:19:56:0.99:0.60:0.33:0.43), (PHENTOLAMINE MESYLATE:26:11209:2:73:0.99:0.93:0.26:0.41), (TRYPTOPHAN:22:11213:13:62:0.99:0.6 3:0.26:0.37), (NALFURAFINE HYDROCHLORIDE:20:11215:13:62:0.99:0.61:0.24:0.35), (AMOXICILLIN SODIUM:19:11216:10:65:0.99:0.66:0.23:0.34), (AMLODIPINE MALEATE:18:11217:5:70:0.99:0.78:0.20:0.32), (CALCIUM ASCORBATE:18:11217:5:70:0.99:0.78:0.20:0.32), (POLYMYXIN:16:11219:14:61:0.99:0.5 3:0.21:0.30), (CEF CAPENE PIVOXIL:14:11221:7:68:0.99:0.67:0.17:0.27), (OXATOMIDE:14:11221:4:71:0.99:0.78:0.16: 0.27), (HYDROCHLOROTHIAZIDENEBIVOLOL HYDROCHLORIDE:9:11226:0:75:0.99:1.00:0.11:0.19), (MAPROTILINE:7:11228:3:72:0.99: 0.70:0.09:0.16), (PYRIDOXINETHIAMINE:7:11228:1:74:0.99:0.88:0.09:0.16), (SODIUM PHENYLBUTYRATE:4:11231:0:75:0.99:1.00:0.05:0.10), (SODIUM BENZOATE:3:11232:1:74:0.99:0.75:0.04:0.07), (NITRIC OXIDE:117:11117:2:74:0.99:0.98:0.61:0.75), (DEFIBROTIDE (BOVINE):38:11196:6:70:0.99:0.86:0.35:0.50), (AMSACRINE:25:11209:3:73:0.99:0.89:0.2 6:0.40), (CALCIUM GLYCEROPHOSPHATE:24:11210:8:68:0.99:0.75:0.26:0.39), (CEFOPERAZONETAZOBA CTAM SODIUM:21:11213:1:75:0.99:0.95:0.22:0.36), (HYDROCORTISONENEOMYCIN SULFATEPOLYMYXIN B SULFATE:16:11218:6:70:0.99:0.73:0.19:0.30), (FAECALIS:15:11219:3:73:0.99:0.83:0.17:0.28), (BARNIDIPINE HYDROCHLORIDE:11:11223:2:74:0.99:0.85:0.13:0.22), (CYSTEINEGLYCINEGLYCYR RHIZIN:9:11225:3:73:0.99:0.75:0.11:0.19), (MIDAZOLAM MALEATE:9:11225:0:76:0.99:1.00:0.11:0.19), (ITOPRIDE HYDROCHLORIDE:8:11226:1:75:0.99:0.89:0.10:0.17), (CHLORPHENIRAMINE MALEATEDEXTROMETHORPHAN HYDROBROMIDE:6:11228:0:76:0.99:1.00:0.07:0.14), (ALFENTANIL HYDROCHLORIDE:4:11230:0:76:0.99:1.00:0.05:0.10), (CHLORMADINONE:4:11230:0:76: 0.99:1.00:0.05:0.10), (THYMALFASIN:4:11230:1:75:0.99:0.80:0.05:0.10), (HYDROXYCH LOROQUINE:1056:10177:0:77:0.99:1.00:0.93:0.96), (TREPROSTINIL DIOLAMINE:49:11184:33:44:0.99:0.60:0.53:0.56), (PIRBUTEROL ACETATE:48:11185:12:65:0.99:0.80:0.42:0.55), (BENIDIPINE:40:11193:23:54:0.99:0.63:0 0.43:0.51), (CEPHARANTHINE:37:11196:35:42:0.99:0.51:0.47:0.49), (PENICILLAMINE:27: 11206:18:59:0.99:0.60:0.31:0.41), (CAMPHOR (NATURAL)MENTHOLPRAMOXINE HYDROCHLORIDE:22:11211:4:73:0.99:0.85:0.23:0.36), (PAZUFLOXACIN MESILATE:22:11211:24:53:0.99:0.48:0.29:0.36), (DICLOFENAC DIETHYLAMINE:18:11215:17:60:0.99:0.51:0.23:0.32), (ESTROGENS, CONJUGATED SYNTHETIC A:13:11220:1:76:0.99:0.93:0.15:0.25), (TRANILAST:13:11220:10:67:0.99:0.57:0.16:0.25), (ETHIODIZED OIL:11:11222:11:66:0.99:0.50:0.14:0.22), (ALUMINUM HYDROXIDEDIMETHICONEDIPHENHYDRAMINELIDOCAINEMAGNESIUM HYDROXIDE:6:11227:0:77:0.99:1.00:0.07:0.13), (KETOPROFEN LYSINE:6:11227:0:77:0.99:1.00:0.07:0.13), (ORNITHINE ASPARTATE:4:11229:2:75:0.99:0.67:0.05:0.09), (TERFENADINE:3:11230:0:77:0.99:1.00: 0.04:0.07), (CAMPHORMENTHOLMETHYL SALICYLATE:34:11198:29:49:0.99:0.54:0.41:0.47), (LINSIDOMINE:33:11199:52:26:0.99: 0.39:0.56:0.46), (IBUDILAST:30:11202:7:71:0.99:0.81:0.30:0.43), (MECLIZINE MONOHYDROCHLORIDENIACIN:28:11204:18:60:0.99:0.61:0.32:0.42), (CERIVASTATI N SODIUM:22:11210:56:22:0.99:0.28:0.50:0.36), (TRIFLUOPERAZINE HYDROCHLORIDE:20:11212:5:73:0.99:0.80:0.22:0.34), (DROSPIRENONEESTRADIOL:1 8:11214:8:70:0.99:0.69:0.20:0.32), (PROPOXYPHENE:17:11215:7:71:0.99:0.71:0.19:0.30), (OPIUM TINCTURE:15:11217:0:78:0.99:1.00:0.16:0.28), (CHLORPROMAZINE HYDROCHLORIDEPHENOBARBITALPROMETHAZINE:14:11218:2:76:0.99:0.88:0.16: 0.26), (TRIAMCINOLONE DIACETATE:12:11220:2:76:0.99:0.86:0.14:0.24), (INCOBOTULINUMTOXINA:11:11221:4: 74:0.99:0.73:0.13:0.22), (DICLOXACILLIN SODIUM:7:11225:4:74:0.99:0.64:0.09:0.15), (NAFCILLINNAFCILLIN SODIUM:7:11225:0:78:0.99:1.00:0.08:0.15), (RINGER'S SOLUTION:5:11227:0:78:0.99:1.00:0.06:0.11), (SODIUM PERCHLORATE MONOHYDRATE:4:11228:0:78:0.99:1.00:0.05:0.09), (BOSENTAN:341:10890:0:79:0.99:1.00:0.81:0.90), (METHENAMINE MANDELATE:23:11208:58:21:0.99:0.28:0.52:0.37), (ASENAPINE:17:11214:8:71:0.99:0.68: 0.19:0.30), (ATOVAQUONEPROGUANIL HYDROCHLORIDE:17:11214:4:75:0.99:0.81:0.18:0.30), (SALICYLAMIDE:13:11218:2:77: 0.99:0.87:0.14:0.25), (CARBOMER 934GLYCERINMETHYLPARABENPROPYLPARABEN\SODIUM HYDROXIDE:12:11219:5:74:0.99:0.71:0.14:0.23), (TEGAFUR:11:11220:3:76:0.99:0.79:0.1 3:0.22), (TRIMETHOBENZAMIDE:11:11220:3:76:0.99:0.79:0.13:0.22), (GRANULOCYTE-MACROPHAGE COLONY-STIMULATING FACTOR:7:11224:1:78:0.99:0.88:0.08:0.15), (CALCIUM CHLORIDE\SODIUM CHLORIDE\SODIUM PHOSPHATE, DIBASIC, ANHYDROUS\SODIUM PHOSPHATE, MONOBASIC, ANHYDROUS:5:11226:0:79:0.99:1.00:0.06:0.11), (CEFOPERAZONE SODIUM:4:11227:1:78:0.99:0.80:0.05:0.09), (LENTINAN:3:11228:1:78:0.99:0.75:0.04:0.0 7), (TESAMORELIN:43:11187:19:61:0.99:0.69:0.41:0.52), (SIBUTRAMINE HYDROCHLORIDE:32:11198:53:27:0.99:0.38:0.54:0.44), (CRANBERRY CONCENTRATE:31:11199:8:72:0.99:0.79:0.30:0.44), (ASPIRINCLOPIDOGREL BISULFATE:30:11200:2:78:0.99:0.94:0.28:0.43), (.BETA.-CAROTENE:27:11203:13:67:0.99:0.68:0.29:0.40), (AMILORIDE HYDROCHLORIDEFUROSEMIDE:26:11204:22:58:0.99:0.54:0.31:0.39), (HYDRALAZINE HYDROCHLORIDEISOSORBIDE DINITRATE:23:11207:7:73:0.99:0.77:0.24:0.37), (PORFIMER SODIUM:23:11207:52:28:0.99:0.31:0.45:0.37), (TIOCONAZOLE:14:11216:3:77:0.99:0.82: 0.15:0.26), (ZUCLOPENTHIXOL ACETATE:12:11218:0:80:0.99:1.00:0.13:0.23), (ADENOSINE TRIPHOSPHATE DISODIUM:10:11220:4:76:0.99:0.71:0.12:0.20), (CILASTATIN:8:11222:1:79:0.99:0.89:0.0 9:0.17), (VILAZODONE:6:11224:0:80:0.99:1.00:0.07:0.13), (MINERAL OILPETROLATUMWAX, EMULSIFYING:5:11225:0:80:0.99:1.00:0.06:0.11), (MOCLOBEMIDE:5:11225:0:80:0.99:1.00:0.06:0.11), (VALPROIC ACID:1129:10100:0:81:0.99:1.00:0.93:0.97), (VINBLASTINE:192:11037:5:76:0.99:0.97:0.7 2:0.83), (FOLIC ACIDVITAMINS:69:11160:0:81:0.99:1.00:0.46:0.63), (IGURATIMOD:40:11189:36:45:0.99: 0.53:0.47:0.50), (PIZOTYLINE:38:11191:17:64:0.99:0.69:0.37:0.48), (ALUMINUM HYDROXIDEMAGNESIUM OXIDEOXETHAZAINE:37:11192:48:33:0.99:0.44:0.53:0.48), (ARSENIC TRIOXIDE:23:11206:16:65:0.99:0.59:0.26:0.36), (ASPARAGINASE CHRYSANTHEMI:23:11206:4:77:0.99:0.85:0.23:0.36), (MAGNESIUM PIDOLATE:23:11206:34:47:0.99:0.40:0.33:0.36), (PROPIOMAZINE:19:11210:4:77:0.99:0.8 3:0.20:0.32), (CEFMETAZOLE:15:11214:8:73:0.99:0.65:0.17:0.27), (AMLODIPINE BESYLATEPERINDOPRIL ARGININE:11:11218:1:80:0.99:0.92:0.12:0.21), (POTASSIUM PHOSPHATE, DIBASIC\POTASSIUM PHOSPHATE, MONOBASIC:10:11219:1:80:0.99:0.91:0.11:0.20), (AMILORIDEFUROSEMIDE:5:11224:0: 81:0.99:1.00:0.06:0.11), (PROPACETAMOL:4:11225:0:81:0.99:1.00:0.05:0.09), (DEXTR OMETHORPHAN HYDROBROMIDEGUAIFENESINPHENYLEPHRINE HYDROCHLORIDE:3:11226:3:78:0.99:0.50:0.04:0.07), (AVANAFIL:65:11163:23:59:0.99: 0.74:0.52:0.61), (EMTRICITABINERILPIVIRINE HYDROCHLORIDETENOFOVIR DISOPROXIL FUMARATE:53:11175:29:53:0.99:0.65:0.50:0.56), (TOFACITINIB:48:11180:16:66:0.99:0.7: 0.42:0.54), (TEDUGLUTIDE:36:11192:32:50:0.99:0.53:0.42:0.47), (ZINC ACETATE:24:11204:10:72:0.99:0.71:0.25:0.37), (CHOLINE BITARTRATECYSTEINEINOSITOLMAMMAL LIVER:19:11209:16:66:0.99:0.54:0.22:0.32), (PLANTAGO OVATA LEAF:18:11210:1:81:0.99:0.95:0.18:0.31), (LEVOBUPIVACAINE HYDROCHLORIDE:16:11212:3:79:0.99:0.84:0.17:0.28), (PHOSPHATIDYL SERINE:10:11218:4:78:0.99:0.71:0.11:0.20), (POTASSIUM ACETATE:9:11219:2:80:0.99:0.82:0.10:0.18), (COENZYME A:8:11220:0:82:0.99:1.00:0.09:0.16), (SULFACETAMIDE:5:11223:0:82:0.99:1.00:0.06:0.1 1), (MEPIVACAINEMEPIVACAINE HYDROCHLORIDE:4:11224:0:82:0.99:1.00:0.05:0.09), (CALCIUM CARBONATEERGOCALCIFEROL:49:11178:20:63:0.99:0.71:0.44:0.54), (ECABET:41:111 86:11:72:0.99:0.79:0.36:0.50), (SULTOPRIDE HYDROCHLORIDE:24:11203:15:68:0.99:0.62:0.26:0.37), (ADENINE:21:11206:54:29:0.99: 0.28:0.42:0.34), (BICARBONATE IONPOLYETHYLENE GLYCOLSPOTASSIUM CATION\SODIUM CHLORIDE\SODIUM SULFATE ANHYDROUS:18:11209:13:70:0.99:0.58:0.20:0.30), (CITICOLINE:18:11209:10:73:0.99:0.6 4:0.20:0.30), (DIBUCAINE HYDROCHLORIDEHYDROCORTISONE:18:11209:1:82:0.99:0.95:0.18:0.30), (BETAMET HASONEBETAMETHASONE DIPROPIONATECLOTRIMAZOLE:17:11210:0:83:0.99:1.00:0.17:0.29), (FLUNARIZINE:1 5:11212:3:80:0.99:0.83:0.16:0.27), (PAMIDRONIC ACID:14:11213:5:78:0.99:0.74:0.15:0.25), (PROTHIPENDYL HYDROCHLORIDE:13:11214:1:82:0.99:0.93:0.14:0.24), (TROXERUTIN:13:11214:2:81:0.9 9:0.87:0.14:0.24), (PERICIAZINE:12:11215:1:82:0.99:0.92:0.13:0.22), (OXYTETRACYCL INE:11:11216:2:81:0.99:0.85:0.12:0.21), (THROMBIN:8:11219:0:83:0.99:1.00:0.09:0.16), (MITOTANE:6:11221:1:82:0.99:0.86:0.07:0.13), (CARRAGEENANTITANIUM\ZINC:5:112 22:2:81:0.99:0.71:0.06:0.11), (FISH OILMEDIUM-CHAIN TRIGLYCERIDESOLIVE OILSOYBEAN OIL:4:11223:0:83:0.99:1.00:0.05:0.09), (NITROPRUSSIDE:4:11223:0:83:0.99:1.00:0.05:0.09), (PENICILLIN G POTASSIUM:3:11224:1:82:0.99:0.75:0.04:0.07), (SUNITINIB:88:11138:10:74:0.99:0.90:0.54:0.68), (DANAPAROID SODIUM:84:11142:2:82:0.99:0.98:0.51:0.67), (VALDECOXIB:45:11181:40:44:0.99:0.53:0.51:0.52), (TIMOLOLTRAVOPROST:39:11187:52:32:0.99:0.43:0.55:0.48), (POLYSTYREN E SULFONIC ACID:37:11189:26:58:0.99:0.59:0.39:0.47), (HORSE CHESTNUT:34:11192:31:53:0.99:0.52:0.39:0.45), (GADOLINIUM:33:11193:21:63:0.99:0.6 1:0.34:0.44), (BETAMIPRONPANIPENEM:32:11194:25:59:0.99:0.56:0.35:0.43), (EMTRI CITABINETENOFOVIR ALAFENAMIDE FUMARATE:25:11201:7:77:0.99:0.78:0.25:0.37), (RHUBARB:20:11206:20:64:0.99:0.50:0.24:0.32), (SODIUM PHOSPHATE, MONOBASIC, ANHYDROUS:15:11211:10:74:0.99:0.60:0.17:0.26), (TETRAFERRIC TRICITRATE DECAHYDRATE:12:11214:0:84:0.99:1.00:0.13:0.22), (CLODRONIC ACID:7:11219:1:83:0.99:0.88:0.08:0.14), (FLUORESCEIN:7:11219:1:83:0.99:0.88:0.08:0.1 4), (CYCLIZINE HYDROCHLORIDE:6:11220:1:83:0.99:0.86:0.07:0.13), (DIPHTHERIA AND TETANUS TOXOIDS AND ACELLULAR PERTUSSIS ADSORBED AND INACTIVATED POLIOVIRUS VACCINE:5:11221:0:84:0.99:1.00:0.06:0.11), (DROTAVERINE HYDROCHLORIDE:4:11222:0:84:0.99:1.00:0.05:0.09), (PEGVISOMANT:137:11088:18:67: 0.99:0.88:0.67:0.76), (TASIMELTEON:64:11161:29:56:0.99:0.69:0.53:0.60), (NEFAZODO NE:44:11181:14:71:0.99:0.76:0.38:0.51), (MOSAPRIDE CITRATE DIHYDRATE:40:11185:17:68:0.99:0.70:0.37:0.48), (NABIXIMOLS:28:11197:7:78:0.99:0.8 0:0.26:0.40), (CARTEOLOL:26:11199:38:47:0.99:0.41:0.36:0.38), (AMPHOTERICIN BDIMYRISTOYLPHOSPHATIDYLCHOLINE, DL-DIMYRISTOYLPHOSPHATIDYLGLYCEROL, DL-:24:11201:8:77:0.99:0.75:0.24:0.36), (MAGNESIUM SULFATE\POTASSIUM SULFATE\SODIUM SULFATE:19:11206:11:74:0.99:0.63:0.20:0.31), (ACETAMINOPHENCHLORPHENIRAMI NE MALEATEPHENYLEPHRINE HYDROCHLORIDE:17:11208:1:84:0.99:0.94:0.17:0.29), (CEFTAROLINE:11:11214:2:83:0. 99:0.85:0.12:0.21), (HONEY:11:11214:2:83:0.99:0.85:0.12:0.21), (COAGULATION FACTOR IX RECOMBINANT HUMANISOPROPYL ALCOHOL:10:11215:4:81:0.99:0.71:0.11:0.19), (CELIPROLOL HYDROCHLORIDE:9:11216:0:85:0.99:1.00:0.10:0.17), (LEVOTHYROXINE SODIUMLIOTHYRONINE SODIUM:8:11217:1:84:0.99:0.89:0.09:0.16), (TOLPERISONE:6:11219:1:84:0.99:0.86:0.07: 0.12), (CHLOROFORMCODEINE PHOSPHATEDRIMIA MARITIMA BULB:4:11221:0:85:0.99:1.00:0.04:0.09), (FISH OILOLIVEOILSOYBEAN OILTRIGLYCERIDES,UNSPECIFIED LENGTH:4:11221:0:85:0.99:1.00:0.04:0.09), (DAUNORUBICIN HYDROCHLORIDE:45:11179:9:77:0.99:0.83:0.37:0.51), (ATORVASTATIN CALCIUMEZETIMIBE:40:11184:18:68:0.99:0.69:0.37:0.48), (POTASSIUM CHLORIDE\SODIUM CHLORIDE\SODIUM LACTATE:37:11187:22:64:0.99:0.63:0.37:0.46), (AZITHROMYCIN OR METHOTREXATE SODIUM:36:11188:50:36:0.99:0.42:0.50:0.46), (DIMEMORFAN PHOSPHATE:35:11189:17:69:0.99:0.67:0.34:0.45), (PIMOZIDE:34:11190:0:86:0.99:1.00:0. 28:0.44), (ESTRADIOLLEVONORGESTREL:29:11195:24:62:0.99:0.55:0.32:0.40), (FLAV OXATE HYDROCHLORIDE:26:11198:12:74:0.99:0.68:0.26:0.38), (TANDOSPIRONE:26:11198:9:7 7:0.99:0.74:0.25:0.38), (BISMUTH SUBCITRATE POTASSIUMMETRONIDAZOLETETRACYCLINE HYDROCHLORIDE:20:11204:5:81:0.99:0.80:0.20:0.32), (ATROPINE SULFATEDIPHENOXYLATE:16:11208:9:77:0.99:0.64:0.17:0.27), (DEXIBUPROFEN:14:1 1210:4:82:0.99:0.78:0.15:0.25), (PLECANATIDE:13:11211:2:84:0.99:0.87:0.13:0.23), (ME BENDAZOLE:8:11216:1:85:0.99:0.89:0.09:0.16), (DARUNAVIR ETHANOLATE:435:10788:2:85:0.99:1.00:0.84:0.91), (POVIDONE:61:11162:36:51:0.99:0.6 3:0.54:0.58), (FOLIC ACIDIRONMINERALSVITAMINS:44:11179:11:76:0.99:0.80:0.37:0.50), (LYSOZYME HYDROCHLORIDE:40:11183:32:55:0.99:0.56:0.42:0.48), (ATOMOXETINE:31:11192:12:7 5:0.99:0.72:0.29:0.42), (PROCARBAZINE HYDROCHLORIDE:29:11194:5:82:0.99:0.85:0.26:0.40), (EUCALYPTOLMENTHOLMET HYL SALICYLATETHYMOL:27:11196:29:58:0.99:0.48:0.32:0.38), (OMEGA-3-CARBOXYLIC ACIDS:21:11202:7:80:0.99:0.75:0.21:0.33), (CROMOLYN:18:11205:2:85:0.99:0.90:0.17:0. 29), (PROCAINE HYDROCHLORIDE:15:11208:5:82:0.99:0.75:0.15:0.26), (SACUBITRIL:13:11210:1:86:0.99: 0.93:0.13:0.23), (CEFOPERAZONESULBACTAM:12:11211:4:83:0.99:0.75:0.13:0.22), (C ITALOPRAM HYDROCHLORIDE:8:11215:1:86:0.99:0.89:0.09:0.16), (AMIFOSTINE:5:11218:1:86:0.99:0 0.83:0.05:0.10), (BAZEDOXIFENE ACETATEESTROGENS, CONJUGATED:47:11175:13:75:0.99:0.78:0.39:0.52), (OLODATEROL HYDROCHLORIDE:33:11189:16:72:0.99:0.67:0.31:0.43), (ILOPERIDONE:32:11190:2:86:0 0.99:0.94:0.27:0.42), (TAZAROTENE:31:11191:13:75:0.99:0.70:0.29:0.41), (FLUNARIZINE HYDROCHLORIDE:28:11194:47:41:0.99:0.37:0.41:0.39), (ALECTINIB:27:11195:18:70:0.9 9:0.60:0.28:0.38), (GARLIC OIL:26:11196:7:81:0.99:0.79:0.24:0.37), (TIAPRIDE:17:11205:10:78:0.99:0.63:0.18:0.28), (DYPHYLLINE:13:11209:2:86:0.99:0.87:0.13:0.23), (COSYNTROPIN:12:11210:4:84:0.99:0 0.75:0.13:0.21), (STRONTIUM RANELATE:11:11211:4:84:0.99:0.73:0.12:0.20), (CEFTOLOZANE SULFATETAZOBACTAM SODIUM:7:11215:0:88:0.99:1.00:0.07:0.14), (FELBAMATE:49:11172:21:68:0.99:0.70:0.42: 0.52), (ESTRADIOL VALERATE:46:11175:2:87:0.99:0.96:0.35:0.51), (AMMONIUM GLYCYRRHIZATECYSTEINE HYDROCHLORIDEGLYCINE:38:11183:18:71:0.99:0.68:0.35:0.46), (SERRAPEPTASE:38: 11183:34:55:0.99:0.53:0.41:0.46), (CALCIUM PANTOTHENATECYANOCOBALAMINNIACINAMIDEPYRIDOXINE HYDROCHLORIDE\RIBOFLAVINTHIAMINE HYDROCHLORIDE:30:11191:9:80:0.99:0.77:0.27:0.40), (DIHYDROERGOTAMINE MESYLATE:28:11193:6:83:0.99:0.82:0.25:0.39), (LONGUMLACTOBACILLUS ACIDOPHILUS:26:11195:9:80:0.99:0.74:0.25:0.37), (NEOSTIGMINE METHYLSULFATE:24:11197:8:81:0.99:0.75:0.23:0.35), (MORNIFLUMATE:23:11198:8:81: 0.99:0.74:0.22:0.34), (BEE POLLEN:22:11199:3:86:0.99:0.88:0.20:0.33), (BENDROFLUMETHIAZIDE\POTASSIUM CHLORIDE:20:11201:0:89:0.99:1.00:0.18:0.31), (ERGOLOID MESYLATES:18:11203:19:70:0.99:0.49:0.20:0.29), (PROMAZINE:18:11203:10:79:0.99:0.6 4:0.19:0.29), (CLOPIDOGREL BESILATE:15:11206:1:88:0.99:0.94:0.15:0.25), (TIOPRONIN:15:11206:20:69:0.99:0.43:0.1 8:0.25), (GADOXETATE DISODIUM:10:11211:5:84:0.99:0.67:0.11:0.18), (METHYLERGONOVINE MALEATE:8:11213:2:87:0.99:0.80:0.08:0.15), (DEXTROSE\POTASSIUM CHLORIDE\SODIUM CHLORIDE:5:11216:0:89:0.99:1.00:0.05:0.10), (CITRULLINE:4:11217:1:88:0.99:0.80:0.04: 0.08), (ASPIRINLANSOPRAZOLE:55:11165:12:78:0.99:0.82:0.41:0.55), (TAFAMIDIS MEGLUMINE:51:11169:31:59:0.99:0.62:0.46:0.53), (CANNABIDIOL:41:11179:12:78:0.99: 0.77:0.34:0.48), (ALUMINUM SILICATE:29:11191:28:62:0.99:0.51:0.32:0.39), (FILGRASTIM-SNDZ:26:11194:11:79:0.99:0.70:0.25:0.37), (PEROSPIRONE HYDROCHLORIDE:26:11194:13:77:0.99:0.67:0.25:0.37), (TINIDAZOLE:25:11195:7:83:0.9 9:0.78:0.23:0.36), (ESTROGENS, ESTERIFIED:23:11197:5:85:0.99:0.82:0.21:0.34), (LEVOMEPROMAZINE MALEATE:19:11201:5:85:0.99:0.79:0.18:0.30), (PROPIVERINE HYDROCHLORIDE:17:11203:2:88:0.99:0.89:0.16:0.27), (FORMOTEROL FUMARATEGLYCOPYRROLATE:16:11204:2:88:0.99:0.89:0.15:0.26), (LANOLIN:7:1121 3:0:90:0.99:1.00:0.07:0.13), (BICYCLOL:49:11170:1:90:0.99:0.98:0.35:0.52), (FURSULTI AMINE HYDROCHLORIDE:44:11175:5:86:0.99:0.90:0.34:0.49), (HYDROXYCITRIC ACID:32:11187:8:83:0.99:0.80:0.28:0.41), (PYRIDOXINE HYDROCHLORIDETHIAMINE HYDROCHLORIDE:14:11205:1:90:0.99:0.93:0.13:0.24), (CALCIUM CITRATECHOLECALCIFEROL:11:11208:1:90:0.99:0.92:0.11:0.19), (METHYLPREDNIS OLONE ACEPONATE:5:11214:0:91:0.99:1.00:0.05:0.10), (THEOBROMINE:4:11215:0:91:0.99:1.00: 0.04:0.08), (AMINOSALICYLIC ACID:53:11165:37:55:0.99:0.59:0.49:0.54), (CAMPHORDIPHENHYDRAMINE\ZINC OXIDE:39:11179:25:67:0.99:0.61:0.37:0.46), (APPLE CIDER VINEGAR:33:11185:6:86:0.99:0.85:0.28:0.42), (FOSAMPRENAVIR CALCIUM:14:11204:1:91:0.99:0.93:0.13:0.23), (CLOTHIAPINE:8:11210:1:91:0.99:0.89:0. 08:0.15), (COLESEVELAM:6:11212:0:92:0.99:1.00:0.06:0.12), (METARAMINOL:6:11212: 6:86:0.99:0.50:0.07:0.12), (THYMOPENTIN:4:11214:0:92:0.99:1.00:0.04:0.08), (OMBITA SVIRPARITAPREVIRRITONAVIR:404:10813:0:93:0.99:1.00:0.81:0.90), (FLAVIN ADENINE DINUCLEOTIDE:56:11161:34:59:0.99:0.62:0.49:0.55), (CALCIUM PANTOTHENATECYANOCOBALAMINNIACINAMIDEPYRIDOXINE HYDROCHLORIDE\RIBOFLAVINTHIAMINE MONONITRATE:45:11172:43:50:0.99:0.51:0.47:0.49), (AJMALINE:41:11176:18:75:0.99:0. 69:0.35:0.47), (IFENPRODIL:41:11176:18:75:0.99:0.69:0.35:0.47), (ZOTEPINE:38:11179:1 4:79:0.99:0.73:0.32:0.45), (TEGAFURURACIL:32:11185:17:76:0.99:0.65:0.30:0.41), (FER RITIN:26:11191:10:83:0.99:0.72:0.24:0.36), (DEXTRAN 70HYPROMELLOSE 2910 (4000 MPA. S):24:11193:3:90:0.99:0.89:0.21:0.34), (PROTAMINE SULFATE:23:11194:2:91:0.99:0.92:0.20:0.33), (CICLOPIROX OLAMINE:20:11197:41:52:0.99:0.33:0.28:0.30), (LANOLINPETROLATUM:13:11204:6:87: 0.99:0.68:0.13:0.22), (CHONDROITIN SULFATE (BOVINE)DIMETHYL SULFONEGLUCOSAMINE SULFATE:11:11206:1:92:0.99:0.92:0.11:0.19), (VINDESINE:11:11206:2:91:0.99:0.85:0.11: 0.19), (DEXRAZOXANE HYDROCHLORIDE:7:11210:1:92:0.99:0.88:0.07:0.13), (BRENTUXIMAB VEDOTIN:217:10999:3:91:0.99:0.99:0.70:0.82), (THIOPENTAL SODIUM:89:11127:23:71:0.99:0.79:0.56:0.65), (BERAPROST:53:11163:46:48:0.99:0.54:0. 52:0.53), (METFORMIN HYDROCHLORIDESAXAGLIPTIN:39:11177:66:28:0.99:0.37:0.58:0.45), (CEFOPERAZO NE:32:11184:5:89:0.99:0.86:0.26:0.41), (SPIRULINA:27:11189:8:86:0.99:0.77:0.24:0.36), (ASCORBIC ACIDFERROUS SULFATE:26:11190:4:90:0.99:0.87:0.22:0.36), (SULBACTAM SODIUM:25:11191:12:82:0.99:0.68:0.23:0.35), (CIPROFLOXACINCIPROFLOXACIN HYDROCHLORIDE:20:11196:2:92:0.99:0.91:0.18:0.30), (CARBOXYMETHYLCELLULOS E:19:11197:1:93:0.99:0.95:0.17:0.29), (DIAMORPHINE:16:11200:4:90:0.99:0.80:0.15:0.25), (GLUCOSAMINE HYDROCHLORIDE:14:11202:6:88:0.99:0.70:0.14:0.23), (CHLORHEXIDINE GLUCONATECHLOROBUTANOL:12:11204:1:93:0.99:0.92:0.11:0.20), (GLYCINE:11:112 05:2:92:0.99:0.85:0.11:0.19), (ELBASVIRGRAZOPREVIR:89:11126:48:47:0.99:0.65:0.65: 0.65), (THIAMYLAL SODIUM:47:11168:33:62:0.99:0.59:0.43:0.50), (PRUCALOPRIDE SUCCINATE:39:11176:26:69:0.99:0.60:0.36:0.45), (HERBALSMINERALSVITAMINS:34: 11181:13:82:0.99:0.72:0.29:0.42), (CYCLOPENTOLATE HYDROCHLORIDE:24:11191:1:94:0.99:0.96:0.20:0.34), (VALBENAZINE:10:11205:1:94:0. 99:0.91:0.10:0.17), (MELPERONE HYDROCHLORIDE:9:11206:1:94:0.99:0.90:0.09:0.16), (HYDROCORTISONEMICONAZ OLE NITRATE:5:11210:0:95:0.99:1.00:0.05:0.10), (RIOCIGUAT:228:10986:38:58:0.99:0.86:0.8 0:0.83), (FLUTICASONE PROPIONATEFORMOTEROL FUMARATE:83:11131:27:69:0.99:0.75:0.55:0.63), (AMOXICILLINCLARITHROMYCIN LANSOPRAZOLE:23:11191:4:92:0.99:0.85:0.20:0.32), (PROCAINE:23:11191:7:89:0.99:0.7 7:0.21:0.32), (CICLOPIROXCICLOPIROX OLAMINE:20:11194:1:95:0.99:0.95:0.17:0.29), (CHOLINE ALFOSCERATE:18:11196:5:91:0.99:0.78:0.17:0.27), (NIACINSIMVASTATIN:15:11199:0: 96:0.99:1.00:0.14:0.24), (TRIMIPRAMINE:12:11202:1:95:0.99:0.92:0.11:0.20), (BORAGE OIL:11:11203:1:95:0.99:0.92:0.10:0.19), (FLAVOXATE:11:11203:0:96:0.99:1.00:0.10:0.19), (HYDROCORTISONE HEMISUCCINATE:10:11204:24:72:0.99:0.29:0.12:0.17), (EPROSARTAN:7:11207:1:95:0.9 9:0.88:0.07:0.13), (CHLOROPYRAMINE HYDROCHLORIDE:4:11210:0:96:0.99:1.00:0.04:0.08), (CLADRIBINE:70:11143:26:71:0.99: 0.73:0.50:0.59), (TILIDINE:60:11153:27:70:0.99:0.69:0.46:0.55), (ATRACURIUM BESYLATE:53:11160:26:71:0.99:0.67:0.43:0.52), (CARBAMIDE PEROXIDE:36:11177:16:81:0.99:0.69:0.31:0.43), (PYRIDOXAL PHOSPHATE:26:11187:30:67:0.99:0.46:0.28:0.35), (SODIUM NITROPRUSSIDE:25:11188:2:95:0.99:0.93:0.21:0.34), (ASPIRINMAGNESIUM HYDROXIDE:24:11189:3:94:0.99:0.89:0.20:0.33), (FLUPENTIXOL DIHYDROCHLORIDEMELITRACEN HYDROCHLORIDE:23:11190:4:93:0.99:0.85:0.20:0.32), (INTERFERON BETA:23:11190:11:86:0.99:0.68:0.21:0.32), (SUPLATAST TOSILATE:23:11190:78:19:0.99:0.23:0.55:0.32), (FLAVOCOXID:22:11191:0:97:0.99:1.00: 0.18:0.31), (VARDENAFIL:12:11201:0:97:0.99:1.00:0.11:0.20), (DEXAMETHASONENE OMYCIN SULFATE:7:11206:1:96:0.99:0.88:0.07:0.13), (ENASIDENIB:6:11207:0:97:0.99:1.00:0.06: 0.11), (FLUOCINOLONE ACETONIDEHYDROQUINONETRETINOIN:3:11210:0:97:0.99:1.00:0.03:0.06), (IVACA FTOR:87:11125:22:76:0.99:0.80:0.53:0.64), (NELARABINE:47:11165:16:82:0.99:0.75:0.36: 0.49), (METFORMIN HYDROCHLORIDEROSIGLITAZONE MALEATE:44:11168:67:31:0.99:0.40:0.59:0.47), (ABIRATERONE:42:11170:11:87:0.99:0.7 9:0.33:0.46), (PREGNENOLONE:38:11174:21:77:0.99:0.64:0.33:0.44), (CALCIUM CARBONATEMAGNESIUM HYDROXIDE:32:11180:5:93:0.99:0.86:0.26:0.40), (TEGASEROD MALEATE:31:11181:13:85:0.99:0.70:0.27:0.39), (TIAGABINE HYDROCHLORIDE:28:11184:9:89:0.99:0.76:0.24:0.36), (HYDROCORTISONE SODIUM PHOSPHATE:20:11192:6:92:0.99:0.77:0.18:0.29), (SULODEXIDE:17:11195:3:95:0.99:0.85: 0.15:0.26), (CALCIUM GLUCONATEDEXTROSEMAGNESIUM SULFATE\POTASSIUM ACETATE\POTASSIUM PHOSPHATE, DIBASICZINC SULFATE:13:11199:1:97:0.99:0.93:0.12:0.21), (ETIFOXINE:13:11199:4:94:0.99:0.76:0.12: 0.21), (POTASSIUM CHLORATE:8:11204:0:98:0.99:1.00:0.08:0.14), (HETASTARCH:6:11206:0:98:0.99:1.00:0. 06:0.11), (CLAVULANATE POTASSIUM:5:11207:0:98:0.99:1.00:0.05:0.09), (LORNOXICAM:86:11125:10:89:0.99:0.9 0:0.49:0.63), (DIPYRONEPROMETHAZINE HYDROCHLORIDE:60:11151:56:43:0.99:0.52:0.58:0.55), (BAMIFYLLINE HYDROCHLORIDE:48:11163:39:60:0.99:0.55:0.44:0.49), (POLYSTYRENE:43:11168:20:79: 0.99:0.68:0.35:0.46), (AMPICILLIN SODIUM:38:11173:43:56:0.99:0.47:0.40:0.43), (PEGINTERFERON ALFA-2A OR PEGINTERFERON ALFA-2B:36:11175:17:82:0.99:0.68:0.31:0.42), (METHAMPHETAMINE:34:11177:0:99:0.99:1.00: 0.26:0.41), (FLUPHENAZINE HYDROCHLORIDE:26:11185:7:92:0.99:0.79:0.22:0.34), (ALCLOMETASONE:21:11190:5: 94:0.99:0.81:0.18:0.30), (POTASSIUM BICARBONATE:17:11194:2:97:0.99:0.89:0.15:0.26), (GRAPE SEED EXTRACT:16:11195:2:97:0.99:0.89:0.14:0.24), (NYSTATINTRIAMCINOLONE:14:11197: 1:98:0.99:0.93:0.13:0.22), (PROTAMINE:9:11202:0:99:0.99:1.00:0.08:0.15), (ORITAVAN CIN:4:11207:0:99:0.99:1.00:0.04:0.07), (DACARBAZINE:195:11015:3:97:0.99:0.98:0.67:0 0.80), (COBICISTATELVITEGRAVIREMTRICITABINETENOFOVIR ALAFENAMIDE FUMARATE:69:11141:31:69:0.99:0.69:0.50:0.58), (PRAMLINTIDE ACETATE:67:11143:20:80:0.99:0.77:0.46:0.57), (ETHYL ICOSAPENTATEICOSAPENT:62:11148:21:79:0.99:0.75:0.44:0.55), (EPALRESTAT:52:11 158:9:91:0.99:0.85:0.36:0.51), (ALOGLIPTIN:37:11173:11:89:0.99:0.77:0.29:0.43), (CLOD RONATE DISODIUM:36:11174:11:89:0.99:0.77:0.29:0.42), (RUFINAMIDE:34:11176:21:79:0.99:0.62: 0.30:0.40), (DEXTRAN:24:11186:10:90:0.99:0.71:0.21:0.32), (GLUCAGON HYDROCHLORIDE:17:11193:3:97:0.99:0.85:0.15:0.25), (AZASETRON HYDROCHLORIDE:13:11197:3:97:0.99:0.81:0.12:0.21), (AZITHROMYCIN:13:11197:5:95: 0.99:0.72:0.12:0.21), (TOLNAFTATE:11:11199:1:99:0.99:0.92:0.10:0.18), (NITAZOXANI DE:10:11200:2:98:0.99:0.83:0.09:0.17), (LOTEPREDNOL ETABONATETOBRAMYCIN:9:11201:0:100:0.99:1.00:0.08:0.15), (ACETIC ACID:8:11202:0:100:0.99:1.00:0.07:0.14), (ALFENTANIL:8:11202:0:100:0.99:1.00:0.07:0. 14), (BENZALKONIUM CHLORIDECHLORHEXIDINE HYDROCHLORIDEISOPROPYL MYRISTATEPARAFFIN:5:11205:0:100:0.99:1.00:0.05:0.09), (DIPHTHERIA AND TETANUS TOXOIDS AND PERTUSSIS VACCINE:5:11205:0:100:0.99:1.00:0.05:0.09), (CALCIUM CARBONATE\SODIUM ALGINATE\SODIUM BICARBONATE:4:11206:1:99:0.99:0.80:0.04:0.07), (FERROUS BISGLYCINATE:49:11160:52:49:0.99:0.49:0.50:0.49), (NEFAZODONE HYDROCHLORIDE:42:11167:20:81:0.99:0.68:0.34:0.45), (LEUCOGEN:41:11168:8:93:0.99: 0.84:0.31:0.45), (LEVAMLODIPINE BESYLATE:40:11169:9:92:0.99:0.82:0.30:0.44), (ALANYL GLUTAMINE:38:11171:1:100:0.99:0.97:0.28:0.43), (ADAPALENEBENZOYL PEROXIDE:32:11177:3:98:0.99:0.91:0.25:0.39), (CEFTAZIDIME SODIUM:32:11177:27:74:0.99:0.54:0.30:0.39), (THIOTHIXENE:29:11180:10:91:0.99:0.74: 0.24:0.36), (BARNIDIPINE:21:11188:3:98:0.99:0.88:0.18:0.29), (OLIVE OIL:19:11190:2:99:0.99:0.90:0.16:0.27), (FUROSEMIDE SODIUM:9:11200:1:100:0.99:0.90:0.08:0.15), (KANAMYCINKANAMYCIN A SULFATE:7:11202:0:101:0.99:1.00:0.06:0.12), (MUPIROCIN CALCIUM:5:11204:0:101:0.99:1.00:0.05:0.09), (AVOBENZONEHOMOSALATEOCTISA LATEOCTOCRYLENEOXYBENZONE:3:11206:0:101:0.99:1.00:0.03:0.06), (SELEGILINE HYDROCHLORIDE:93:11115:29:73:0.99:0.76:0.56:0.65), (DROSPIRENONEETHINYL ESTRADIOLLEVOMEFOLATE CALCIUM:67:11141:11:91:0.99:0.86:0.42:0.57), (SCOPOLAMINE HYDROBROMIDE:59:11149:22:80:0.99:0.73:0.42:0.54), (BENSERAZIDE:31:11177:2:100: 0.99:0.94:0.24:0.38), (LONGUM:27:11181:12:90:0.99:0.69:0.23:0.35), (FERRIC PYROPHOSPHATE:25:11183:5:97:0.99:0.83:0.20:0.33), (MIDOSTAURIN:13:11195:3:99:0. 99:0.81:0.12:0.20), (METHIONINE:8:11200:2:100:0.99:0.80:0.07:0.14), (PSEUDOEPHED RINE HYDROCHLORIDETRIPROLIDINE HYDROCHLORIDE:8:11200:0:102:0.99:1.00:0.07:0.14), (ORNIDAZOLE:4:11204:0:102:0.9 9:1.00:0.04:0.07), (RADIUM RA-223 DICHLORIDE:129:11078:16:87:0.99:0.89:0.60:0.71), (GLIPIZIDEMETFORMIN HYDROCHLORIDE:74:11133:47:56:0.99:0.61:0.57:0.59), (DILAZEP:59:11148:41:62:0.99: 0.59:0.49:0.53), (FROVATRIPTAN SUCCINATE:58:11149:29:74:0.99:0.67:0.44:0.53), (FLUOXETINE HYDROCHLORIDEOLANZAPINE:51:11156:22:81:0.99:0.70:0.39:0.50), (BOSUTINIB:49: 11158:30:73:0.99:0.62:0.40:0.49), (KALLIDINOGENASE:48:11159:17:86:0.99:0.74:0.36:0 0.48), (ZIV-AFLIBERCEPT:47:11160:69:34:0.99:0.41:0.58:0.48), (METHYLENE BLUE:41:11166:21:82:0.99:0.66:0.33:0.44), (ISOLEUCINELEUCINEVALINE:36:11171:5: 98:0.99:0.88:0.27:0.41), (ACETYLCARNITINE:27:11180:1:102:0.99:0.96:0.21:0.34), (EPI NEPHRINELIDOCAINE:23:11184:2:101:0.99:0.92:0.19:0.31), (ASCORBIC ACIDMINERALSVITAMINS:11:11196:2:101:0.99:0.85:0.10:0.18), (GLATIRAMER ACETATE:449:10757:0:104:0.99:1.00:0.81:0.90), (BUPIVACAINE:364:10842:0:104:0.99:1. 00:0.78:0.88), (ANTIHEMOPHILIC FACTOR HUMAN:36:11170:0:104:0.99:1.00:0.26:0.41), (MIGLUSTAT:29:11177:63:41:0.99:0.32:0.4 1:0.36), (IDURSULFASE:25:11181:2:102:0.99:0.93:0.20:0.32), (LEUPROLIDE:14:11192:3: 101:0.99:0.82:0.12:0.21), (EGG PHOSPHOLIPIDSGLYCERINSOYBEAN OIL:4:11202:0:104:0.99:1.00:0.04:0.07), (SILDENAFIL:674:10531:0:105:0.99:1.00:0.87:0. 93), (FOSCARNET:87:11118:1:104:0.99:0.99:0.46:0.62), (DEGARELIX ACETATE:55:11150:16:89:0.99:0.77:0.38:0.51), (CYCLOPENTOLATE:41:11164:16:89:0.9 9:0.72:0.32:0.44), (PIRARUBICIN:38:11167:6:99:0.99:0.86:0.28:0.42), (IMIDAPRIL HYDROCHLORIDE:33:11172:10:95:0.99:0.77:0.26:0.39), (INDACATEROL MALEATE:31:11174:14:91:0.99:0.69:0.25:0.37), (ALOGLIPTIN BENZOATEPIOGLITAZONE HYDROCHLORIDE:30:11175:2:103:0.99:0.94:0.23:0.36), (GLIMEPIRIDEMETFORMIN:2 6:11179:9:96:0.99:0.74:0.21:0.33), (AMPHETAMINE SULFATEDEXTROAMPHETAMINE:11:11194:2:103:0.99:0.85:0.10:0.17), (METAMIZOL E MAGNESIUM:10:11195:2:103:0.99:0.83:0.09:0.16), (TITANIUM DIOXIDE\ZINC OXIDE:6:11199:0:105:0.99:1.00:0.05:0.10), (NAPROXEN SODIUMSUMATRIPTAN SUCCINATE:59:11145:12:94:0.99:0.83:0.39:0.53), (AMINO ACIDSCARBOHYDRATESMINERALSSOYBEAN OILVITAMINS:48:11156:24:82:0.99:0.67:0.37:0.48), (PERMETHRIN:43:11161:7:99:0.99 0.86:0.30:0.45), (PENICILLIN V BENZATHINE:35:11169:17:89:0.99:0.67:0.28:0.40), (NAFTIFINE HYDROCHLORIDE:28:11176:9:97:0.99:0.76:0.22:0.35), (ZANAMIVIR:22:11182:7:99:0.99: 0.76:0.18:0.29), (ETHINYL ESTRADIOLLEVONORGESTREL ACETATE:18:11186:5:101:0.99:0.78:0.15:0.25), (DEXTROMETHORPHAN HYDROBROMIDEPROMETHAZINE HYDROCHLORIDE:17:11187:1:105:0.99:0.94:0.14:0.24), (ANETHOLTRITHION:12:11192: 0:106:0.99:1.00:0.10:0.18), (CALCIUM CHLORIDE\POTASSIUM CHLORIDE\SODIUM CHLORIDE:12:11192:1:105:0.99:0.92:0.10:0.18), (BARIUM SULFATE:11:11193:0:106:0.99:1.00:0.09:0.17), (CALAMINEFERRIC OXIDE REDZINC OXIDE:9:11195:0:106:0.99:1.00:0.08:0.15), (DIATRIZOATE MEGLUMINEDIATRIZOATE SODIUM:8:11196:0:106:0.99:1.00:0.07:0.13), (BIFONAZOLE:5:11199:1:105:0.99:0.83:0.0 5:0.09), (TRIPTORELIN ACETATE:4:11200:0:106:0.99:1.00:0.04:0.07), (BLEOMYCIN SULFATE:298:10905:0:107:0.99:1.00:0.74:0.85), (TIMOLOL MALEATETRAVOPROST:70:11133:50:57:0.99:0.58:0.55:0.57), (MENTHOLZINC OXIDE:55:11148:20:87:0.99:0.73:0.39:0.51), (ERYTHROMYCIN LACTOBIONATE:46:11157:20:87:0.99:0.70:0.35:0.46), (ISONIAZIDPYRAZINAMIDERI FAMPIN:45:11158:30:77:0.99:0.60:0.37:0.46), (PLERIXAFOR:41:11162:12:95:0.99:0.77:0. 30:0.43), (ALCAFTADINE:38:11165:2:105:0.99:0.95:0.27:0.42), (PENTAZOCINE LACTATE:37:11166:6:101:0.99:0.86:0.27:0.41), (ESTRADIOL CYPIONATE:32:11171:3:104:0.99:0.91:0.24:0.37), (INSULIN PORKINSULIN PURIFIED PORK:32:11171:61:46:0.99:0.34:0.41:0.37), (MEPREDNISONE:25:11178:5:102:0.99:0.83:0 0.20:0.32), (ZOFENOPRIL:20:11183:4:103:0.99:0.83:0.16:0.27), (MEPIVACAINE HYDROCHLORIDE:16:11187:2:105:0.99:0.89:0.13:0.23), (PANTOTHENIC ACID:14:11189:1:106:0.99:0.93:0.12:0.21), (ACETAMINOPHENCHLORPHENIRAMINE MALEATE:10:11193:0:107:0.99:1.00:0.09:0.16), (SODIUM ALGINATE\SODIUM BICARBONATE:9:11194:0:107:0.99:1.00:0.08:0.14), (PAPAVERINE:4:11199:0:107:0.99:1. 00:0.04:0.07), (CARMUSTINE:115:11087:30:78:0.99:0.79:0.60:0.68), (GADOPENTETATE DIMEGLUMINE:73:11129:7:101:0.99:0.91:0.42:0.57), (DIFLUNISAL:70:11132:41:67:0.99: 0.63:0.51:0.56), (ENALAPRIL MALEATELERCANIDIPINE HYDROCHLORIDE:63:11139:34:74:0.99:0.65:0.46:0.54), (CARTEOLOL HYDROCHLORIDE:61:11141:40:68:0.99:0.60:0.47:0.53), (LEVOCABASTINE HYDROCHLORIDE:59:11143:20:88:0.99:0.75:0.40:0.52), (CELLULASEDIASTASELIPA SE:45:11157:15:93:0.99:0.75:0.33:0.45), (HIGH DENSITY POLYETHYLENE:18:11184:22:86:0.99:0.45:0.17:0.25), (BACITRACIN ZINCHYDROCORTISONENEOMYCIN SULFATEPOLYMYXIN B SULFATE:9:11193:0:108:0.99:1.00:0.08:0.14), (POTASSIUM BICARBONATE\SODIUM ALGINATE:9:11193:0:108:0.99:1.00:0.08:0.14), (LAMIVUDINE:635:10566:0:109:0.99:1.0 0:0.85:0.92), (DEFEROXAMINE MESYLATE:82:11119:9:100:0.99:0.90:0.45:0.60), (BLONANSERIN:46:11155:21:88:0.99:0. 69:0.34:0.46), (ANTI-INHIBITOR COAGULANT COMPLEX:44:11157:1:108:0.99:0.98:0.29:0.45), (OLANZAPINE PAMOATE:35:11166:10:99:0.99:0.78:0.26:0.39), (GONADOTROPHIN, CHORIONIC:31:11170:0:109:0.99:1.00:0.22:0.36), (CEFOTIAM:19:11182:2:107:0.99:0.90: 0.15:0.26), (SOMATOSTATIN:17:11184:20:89:0.99:0.46:0.16:0.24), (LUTETIUM LU-177:12:11189:93:16:0.99:0.11:0.43:0.18), (EPINEPHRINE BITARTRATELIDOCAINE HYDROCHLORIDE:7:11194:0:109:0.99:1.00:0.06:0.11), (ISOPROPYL MYRISTATEPARAFFIN:7:11194:1:108:0.99:0.88:0.06:0.11), (ZIDOVUDINE:200:11000:3: 107:0.99:0.99:0.65:0.78), (ETRAVIRINE:199:11001:0:110:0.99:1.00:0.64:0.78), (BELATA CEPT:95:11105:83:27:0.99:0.53:0.78:0.63), (ETHINYL ESTRADIOLGESTODENE:58:11142:23:87:0.99:0.72:0.40:0.51), (CHLORPHENIRAMINE MALEATDIHYDROCODEINE PHOSPHATEMETHYLEPHEDRINE HYDROCHLORIDE, DL-:42:11158:9:101:0.99:0.82:0.29:0.43), (ADEMETIONINE SULFATE TOSILATE:28:11172:2:108:0.99:0.93:0.21:0.34), (MALTOSE:16:11184:4:106:0.99:0.80:0.1 3:0.23), (DANTROLENE:15:11185:1:109:0.99:0.94:0.12:0.21), (ISOPROPYL ALCOHOL:15:11185:4:106:0.99:0.79:0.12:0.21), (APOMORPHINE HYDROCHLORIDE:13:11187:1:109:0.99:0.93:0.11:0.19), (ERDOSTEINE:13:11187:1:109:0 0.99:0.93:0.11:0.19), (ALMAGATE:4:11196:0:110:0.99:1.00:0.04:0.07), (TOPOTECAN HYDROCHLORIDE:138:11061:1:110:0.99:0.99:0.56:0.71), (CLOMIPHENE CITRATE:60:11139:1:110:0.99:0.98:0.35:0.52), (FERRIC CITRATE ANHYDROUS:57:11142:11:100:0.99:0.84:0.36:0.51), (LULICONAZOLE:51:11148:12:99:0. 99:0.81:0.34:0.48), (GALANTAMINE HYDROBROMIDE:26:11173:1:110:0.99:0.96:0.19:0.32), (HYDROCORTISONE VALERATE:25:11174:5:106:0.99:0.83:0.19:0.31), (BETAMETHASONE DIPROPIONATEBETAMETHASONE SODIUM PHOSPHATE:19:11180:6:105:0.99:0.76:0.15:0.26), (PENCICLOVIR:19:11180:1:110:0.99:0. 95:0.15:0.26), (LYMECYCLINE:7:11192:0:111:0.99:1.00:0.06:0.11), (EMTRICITABINE:1 75:11023:7:105:0.99:0.96:0.63:0.76), (FLUCYTOSINE:54:11144:21:91:0.99:0.72:0.37:0.49), (TEMOCAPRIL HYDROCHLORIDE:54:11144:21:91:0.99:0.72:0.37:0.49), (COLCHICINEDICYCLOMINE HYDROCHLORIDE:46:11152:19:93:0.99:0.71:0.33:0.45), (FAROPENEM SODIUM:46:11152:29:83:0.99:0.61:0.36:0.45), (ZEAXANTHIN:39:11159:8:104:0.99:0.83: 0.27:0.41), (TETRAZEPAM:37:11161:9:103:0.99:0.80:0.26:0.40), (PREDNISOLONE VALERATE ACETATE:34:11164:10:102:0.99:0.77:0.25:0.38), (CYANOCOBALAMINLIDOCAINE HYDROCHLORIDEPYRIDOXINE HYDROCHLORIDETHIAMINE HYDROCHLORIDE:28:11170:12:100:0.99:0.70:0.22:0.33), (NOMEGESTROL ACETATE:20:11178:2:110:0.99:0.91:0.15:0.26), (CALCIUM\MAGNESIUMVITAMIN DZINC:17:11181:1:111:0.99:0.94:0.13:0.23), (THIORIDAZINE:13:11185:3:109:0.99:0.81: 0.11:0.19), (OXAPROZIN:110:11087:18:95:0.99:0.86:0.54:0.66), (IMIDAFENACIN:45:111 52:5:108:0.99:0.90:0.29:0.44), (SODIUM BICARBONATE\SODIUM CHLORIDE:45:11152:11:102:0.99:0.80:0.31:0.44), (LOPRAZOLAM:40:11157:5:108:0.99:0. 89:0.27:0.41), (MAGNESIUMPYRIDOXINE:30:11167:8:105:0.99:0.79:0.22:0.35), (ISOPR OTERENOL HYDROCHLORIDE:9:11188:2:111:0.99:0.82:0.08:0.14), (NEDAPLATIN:8:11189:2:111:0.9 9:0.80:0.07:0.12), (MITOMYCIN:127:11069:1:113:0.99:0.99:0.53:0.69), (PEGZEREPOETIN ALFA:56:11140:27:87:0.99:0.67:0.39:0.50), (METRELEPTIN:41:11155:64:50:0.99:0.39:0.4 5:0.42), (DYDROGESTERONE:31:11165:10:104:0.99:0.76:0.23:0.35), (ALUMINUM HYDROXIDECALCIUM CARBONATEDIMETHICONEMAGNESIUM HYDROXIDE:22:11174:1:113:0.99:0.96:0.16:0.28), (RILUZOLE:12:11184:2:112:0.99:0.86: 0.10:0.17), (BEDAQUILINE:8:11188:0:114:0.99:1.00:0.07:0.12), (CALCIUM CHLORIDEMAGNESIUM CHLORIDE\POTASSIUM CHLORIDE\SODIUM ACETATE\SODIUM CHLORIDE:7:11189:2:112:0.99:0.78:0.06:0.11), (DOXYLAMINE:6:11190:0:114:0.99:1.00: 0.05:0.10), (VILANTEROL TRIFENATATE:3:11193:0:114:0.99:1.00:0.03:0.05), (NALBUPHINE HYDROCHLORIDE:64:11131:21:94:0.99:0.75:0.41:0.53), (ZALTOPROFEN:47:11148:18:97: 0.99:0.72:0.33:0.45), (POLYVINYL ALCOHOLPOVIDONE:42:11153:8:107:0.99:0.84:0.28:0.42), (PROMAZINE HYDROCHLORIDE:33:11162:13:102:0.99:0.72:0.24:0.36), (NYSTATINTRIAMCINOLONE ACETONIDE:27:11168:3:112:0.99:0.90:0.19:0.32), (NADIFLOXACIN:25:11170:5:110:0.99: 0.83:0.19:0.30), (PEPPERMINT OIL:20:11175:3:112:0.99:0.87:0.15:0.26), (REGADENOSON:15:11180:6:109:0.99:0.71:0.1 2:0.21), (VALPROMIDE:7:11188:0:115:0.99:1.00:0.06:0.11), (XYLOMETAZOLINE HYDROCHLORIDE:4:11191:1:114:0.99:0.80:0.03:0.07), (SELEXIPAG:128:11066:101:15:0. 99:0.56:0.90:0.69), (RUCAPARIB CAMSYLATE:56:11138:36:80:0.99:0.61:0.41:0.49), (TBO-FILGRASTIM:45:11149:11:105:0.99:0.80:0.30:0.44), (TIQUIZIUM BROMIDE:31:11163:8:108:0.99:0.79:0.22:0.35), (POTASSIUM PHOSPHATE\SODIUM PHOSPHATE:24:11170:4:112:0.99:0.86:0.18:0.29), (CARBASPIRIN:19:11175:5:111:0.99:0. 79:0.15:0.25), (DIACEREIN:18:11176:7:109:0.99:0.72:0.14:0.24), (CODEINEGUAIFENES IN:17:11177:1:115:0.99:0.94:0.13:0.23), (LACTOSE:16:11178:2:114:0.99:0.89:0.12:0.22), (ARIPIPRAZOLE LAUROXIL:57:11136:95:22:0.99:0.38:0.72:0.49), (LAPATINIB:54:11139:15:102:0.99:0.78: 0.35:0.48), (CIFENLINE:53:11140:26:91:0.99:0.67:0.37:0.48), (BUPRENORPHINENALO XONE:45:11148:41:76:0.99:0.52:0.37:0.43), (PROBUCOL:44:11149:22:95:0.99:0.67:0.32:0 0.43), (STREPTOMYCIN\STREPTOMYCIN SULFATE:37:11156:24:93:0.99:0.61:0.28:0.39), (BIAPENEM:31:11162:4:113:0.99:0.89:0.2 2:0.35), (RASAGILINE:30:11163:11:106:0.99:0.73:0.22:0.34), (DOXYCYCLINE HYDROCHLORIDE:25:11168:12:105:0.99:0.68:0.19:0.30), (PYGEUM:23:11170:8:109:0.99: 0.74:0.17:0.28), (ACAMPROSATE CALCIUM:16:11177:1:116:0.99:0.94:0.12:0.21), (ICODEXTRIN:9:11184:0:117:0.99:1.00:0 0.07:0.13), (ROPIVACAINE:77:11115:25:93:0.99:0.75:0.45:0.57), (ZICONOTIDE ACETATE:64:11128:40:78:0.99:0.62:0.45:0.52), (ALUMINUM HYDROXIDEMAGNESIUM HYDROXIDESIMETHICONE:50:11142:20:98:0.99:0.71:0.34:0.46), (MINERAL OILPETROLATUM:50:11142:3:115:0.99:0.94:0.30:0.46), (PARATHYROID HORMONE:50:11142:64:54:0.99:0.44:0.48:0.46), (QUAZEPAM:41:11151:8:110:0.99:0.84: 0.27:0.41), (EPRAZINONE:34:11158:10:108:0.99:0.77:0.24:0.37), (DIQUAFOSOL TETRASODIUM:32:11160:6:112:0.99:0.84:0.22:0.35), (SIVELESTAT SODIUM:28:11164:22:96:0.99:0.56:0.23:0.32), (GOSERELIN:17:11175:0:118:0.99:1.00:0.1 3:0.22), (LEVOCETIRIZINE HYDROCHLORIDE:15:11177:1:117:0.99:0.94:0.11:0.20), (PERFLUTREN:12:11180:1:117:0 0.99:0.92:0.09:0.17), (COBAMAMIDE:10:11182:2:116:0.99:0.83:0.08:0.14), (PROCYCLIDI NE:46:11145:14:105:0.99:0.77:0.30:0.44), (AVOCADO OILSOYBEAN OIL:40:11151:14:105:0.99:0.74:0.28:0.40), (ELCATONIN:39:11152:10:109:0.99:0.80:0.26: 0.40), (DOXERCALCIFEROL:31:11160:6:113:0.99:0.84:0.22:0.34), (LYCOPENE:22:11169: 10:109:0.99:0.69:0.17:0.27), (DIFLORASONE DIACETATE:12:11179:0:119:0.99:1.00:0.09:0.17), (METHYLCELLULOSES:11:11180:0:11 9:0.99:1.00:0.08:0.16), (SIPULEUCEL-T:82:11108:61:59:0.99:0.57:0.58:0.58), (PIMOBENDAN:73:11117:75:45:0.99:0.49:0.62:0.5), (LEVOCARNITINE HYDROCHLORIDE:65:11125:21:99:0.99:0.76:0.40:0.52), (ALMOTRIPTAN MALATE:42:11148:7:113:0.99:0.86:0.27:0.41), (GLUTAMINE\SODIUM GUALENATE:39:11151:9:111:0.99:0.81:0.26:0.39), (LONGUMLACTOBACILLUS ACIDOPHILUSLACTOBACILLUS RHAMNOSUS:38:11152:8:112:0.99:0.83:0.25:0.39), (PHENOTHIAZINE:28:11162:12:108:0 0.99:0.70:0.21:0.32), (IRON DEXTRAN:15:11175:3:117:0.99:0.83:0.11:0.20), (CUPRIC SULFATEFERRIC CHLORIDEMANGANESE CHLORIDE\POTASSIUM IODIDE\ZINC SULFATE:14:11176:7:113:0.99:0.67:0.11:0.19), (BALSAM PERUBENZYL BENZOATEBISMUTH HYDROXIDEBISMUTH SUBGALLATEHYDROCORTISONE ACETATE\ZINC OXIDE:13:11177:0:120:0.99:1.00:0.10:0.18), (TROPICAMIDE:127:11062:0:121:0.99:1.00: 0.51:0.68), (ATRACURIUM:63:11126:39:82:0.99:0.62:0.43:0.51), (CLOSTRIDIUM TETANI TOXOID ANTIGEN (FORMALDEHYDE INACTIVATED):41:11148:5:116:0.99:0.89:0.26:0.40), (OXITRIPTAN:40:11149:5:116:0.99: 0.89:0.26:0.40), (QUINACRINE:35:11154:97:24:0.99:0.27:0.59:0.37), (PHENELZINE SULFATE:32:11157:6:115:0.99:0.84:0.22:0.35), (AZILSARTAN KAMEDOXOMILCHLORTHALIDONE:29:11160:0:121:0.99:1.00:0.19:0.32), (FLURAND RENOLIDE:22:11167:7:114:0.99:0.76:0.16:0.27), (ETILEFRINE HYDROCHLORIDE:19:11170:4:117:0.99:0.83:0.14:0.24), (BENFOTIAMINE:15:11174:4:11 7:0.99:0.79:0.11:0.20), (DEFIBROTIDE SODIUM:8:11181:0:121:0.99:1.00:0.06:0.12), (LEVODROPROPIZINE:4:11185:0:121:0.99: 1.00:0.03:0.06), (HYDROCHLOROTHIAZIDEQUINAPRIL HYDROCHLORIDE:91:11097:16:106:0.99:0.85:0.46:0.60), (DEXPANTHENOLNIACINA MIDEPYRIDOXINE HYDROCHLORIDE\RIBOFLAVIN 5′-PHOSPHATE SODIUMTHIAMINE HYDROCHLORIDE:67:11121:10:112:0.99:0.87:0.37:0.52), (IRONVITAMINS:60:11128:10: 112:0.99:0.86:0.35:0.50), (FLUPHENAZINE:52:11136:28:94:0.99:0.65:0.36:0.46), (HEPAT ITIS B VIRUS VACCINE:25:11163:3:119:0.99:0.89:0.17:0.29), (RICOLINOSTAT:18:11170:2:120:0.99:0.9 0:0.13:0.23), (MAGNESIUM LACTATE:12:11176:1:121:0.99:0.92:0.09:0.16), (CALCIUM CHLORIDEICODEXTRINMAGNESIUM CHLORIDE\SODIUM CHLORIDE\SODIUM LACTATE:127:11060:2:121:0.99:0.98:0.51:0.67), (BEPRIDIL:78:11109:62:61:0.99:0.56:0.5 6:0.56), (BOTULINUM TOXIN TYPE A:62:11125:54:69:0.99:0.53:0.47:0.50), (AMOXAPINE:55:11132:51:72:0.99:0.52:0.43:0.47), (IMIGLUCERASE:53:11134:75:48:0.99:0.41:0.52:0.46), (EPINASTINE:27:11160:2:121:0.9 9:0.93:0.18:0.31), (CIPROFIBRATE:174:11012:38:86:0.99:0.82:0.67:0.74), (BUCILLAMI NE:89:11097:17:107:0.99:0.84:0.45:0.59), (MELPERONE:76:11110:44:80:0.99:0.63:0.49:0. 55), (FERRIC OXYHYDROXIDE:55:11131:15:109:0.99:0.79:0.34:0.47), (CHLORPROTHIXENE:37:11149: 20:104:0.99:0.65:0.26:0.37), (CHLORAL HYDRATE:28:11158:8:116:0.99:0.78:0.19:0.31), (VINBLASTINE SULFATE:27:11159:10:114:0.99:0.73:0.19:0.30), (GELATIN:19:11167:6:118:0.99:0.76:0.1 4:0.23), (TILIDINE HYDROCHLORIDE:18:11168:4:120:0.99:0.82:0.13:0.23), (CELIPROLOL:68:11117:22:103: 0.99:0.76:0.40:0.52), (NISOLDIPINE:61:11124:13:112:0.99:0.82:0.35:0.49), (PROPIVERI NE:56:11129:11:114:0.99:0.84:0.33:0.47), (FIBRINOGEN HUMAN:54:11131:18:107:0.99:0.75:0.34:0.46), (AMLODIPINE BESYLATECANDESARTAN CILEXETIL:53:11132:7:118:0.99:0.88:0.31:0.46), (SULFACETAMIDE SODIUM:31:11154:4:121:0.99:0.89:0.20:0.33), (BERBERINE:29:11156:2:123:0.99:0.94:0.1 9:0.32), (BETAXOLOL:23:11162:0:125:0.99:1.00:0.16:0.27), (COCOA BUTTERPETROLATUMPHENYLEPHRINE HYDROCHLORIDEPHENYLEPHRINE HYDROCHLORIDE OR MINERAL OIL:19:11166:2:123:0.99:0.90:0.13:0.23), (NEOMYCIN SULFATE:13:11172:1:124:0.99:0.93:0.09:0.17), (PENICILLIN G BENZATHINE:11:11174:2:123:0.99:0.85:0.08:0.15), (CEFTAROLINE FOSAMIL:5:11180:0:125:0.99:1.00:0.04:0.07), (.ALPHA.-GLUCOSE-1-PHOSPHATE:4:11181:0:125:0.99:1.00:0.03:0.06), (CAFFEINEDIPYRONEORPHENADRI NE:54:11130:11:115:0.99:0.83:0.32:0.46), (DIETARY SUPPLEMENTHERBALSMILK THISTLE:43:11141:6:120:0.99:0.88:0.26:0.41), (MANIDIPINE HYDROCHLORIDE:41:11143:13:113:0.99:0.76:0.27:0.39), (PINAVERIUM BROMIDE:40:11144:8:118:0.99:0.83:0.25:0.39), (AROTINOLOL HYDROCHLORIDE:17:11167:0:126:0.99:1.00:0.12:0.21), (CRISABOROLE:3:11181:0:126: 0.99:1.00:0.02:0.05), (PIRITRAMIDE:72:11111:26:101:0.99:0.73:0.42:0.53), (RILPIVIRINE HYDROCHLORIDE:64:11119:59:68:0.99:0.52:0.48:0.50), (CEFPROZIL:52:11131:44:83:0.9 9:0.54:0.39:0.45), (HUMAN HEPATITIS B VIRUS IMMUNE GLOBULIN:50:11133:3:124:0.99:0.94:0.29:0.44), (ANAGRELIDE:47:11136:24:103:0.99:0. 66:0.31:0.43), (INDAPAMIDEPERINDOPRIL ERBUMINE:18:11165:1:126:0.99:0.95:0.13:0.22), (XYLOMETAZOLINE:17:11166:2:125:0. 99:0.89:0.12:0.21), (LICORICE:13:11170:0:127:0.99:1.00:0.09:0.17), (BISULEPIN:5:11178: 0:127:0.99:1.00:0.04:0.07), (DEXTROMETHORPHANGUAIFENESIN:5:11178:0:127:0.99: 1.00:0.04:0.07), (MOEXIPRIL HYDROCHLORIDE:72:11110:76:52:0.99:0.49:0.58:0.53), (IDARUBICIN HYDROCHLORIDE:65:11117:6:122:0.99:0.92:0.35:0.50), (DEFERIPRONE:61:11121:32:96: 0.99:0.66:0.39:0.49), (DABIGATRAN ETEXILATE:50:11132:9:119:0.99:0.85:0.30:0.44), (LOMUSTINE:46:11136:36:92:0.99:0.56: 0.33:0.42), (PROTRIPTYLINE HYDROCHLORIDE:37:11145:114:14:0.99:0.25:0.73:0.37), (GLASDEGIB:9:11173:0:128:0. 99:1.00:0.07:0.12), (OPIPRAMOL:82:11099:29:100:0.99:0.74:0.45:0.56), (ETHACRYNIC ACID:71:11110:49:80:0.99:0.59:0.47:0.52), (AMMONIUM CHLORIDEDEXTROMETHORPHAN\SODIUM CITRATE:62:11119:55:74:0.99:0.53:0.46:0.49), (XIPAMIDE:59:11122:13:116:0.99:0.82:0.3 4:0.48), (CHOLESTEROL:58:11123:21:108:0.99:0.73:0.35:0.47), (CYPROTERONE ACETATE:48:11133:13:116:0.99:0.79:0.29:0.43), (POLYMYXIN B SULFATE:39:11142:11:118:0.99:0.78:0.25:0.38), (RISEDRONIC ACID:36:11145:20:109:0.99:0.64:0.25:0.36), (TELOTRISTAT ETHYL:33:11148:91:38:0.99:0.27:0.46:0.34), (LOBUCAVIR:31:11150:5:124:0.99:0.86:0.20: 0.32), (INOSITOL:26:11155:0:129:0.99:1.00:0.17:0.29), (AZULENE:21:11160:1:128:0.99:0. 95:0.14:0.25), (OPIUM:15:11166:1:128:0.99:0.94:0.10:0.19), (POTASSIUM BITARTRATE\SODIUM BICARBONATE:9:11172:0:129:0.99:1.00:0.07:0.12), (GLYBURIDEMETFORMIN HYDROCHLORIDE:169:11011:7:123:0.99:0.96:0.58:0.72), (ATAZANAVIR:125:11055:24:1 06:0.99:0.84:0.54:0.66), (BENSERAZIDELEVODOPA:116:11064:20:110:0.99:0.85:0.51:0 0.64), (THIOCTIC ACID:75:11105:31:99:0.99:0.71:0.43:0.54), (SITAFLOXACIN:38:11142:19:111:0.99:0.67:0 0.26:0.37), (PYRIMETHAMINE:37:11143:36:94:0.99:0.51:0.28:0.36), (BESIFLOXACIN:36: 11144:0:130:0.99:1.00:0.22:0.36), (PAROXETINE MESYLATE:35:11145:3:127:0.99:0.92:0.22:0.35), (HYDROGEN PEROXIDE:27:11153:2:128:0.99:0.93:0.17:0.29), (MILNACIPRAN:26:11154:7:123:0.99:0.7 9:0.17:0.29), (CETYLPYRIDINIUM CHLORIDE:10:11170:0:130:0.99:1.00:0.07:0.13), (OLMESARTAN:311:10868:36:95:0.99:0. 90:0.77:0.83), (DESOGESTRELETHINYL ESTRADIOL:244:10935:7:124:0.99:0.97:0.66:0.79), (TENOFOVIR:230:10949:6:125:0.99:0. 97:0.65:0.78), (TOSUFLOXACIN TOSYLATE:52:11127:14:117:0.99:0.79:0.31:0.44), (ACETAMINOPHENBUTALBITALC AFFEINECODEINE PHOSPHATE:47:11132:8:123:0.99:0.85:0.28:0.42), (DIHYDROCODEINE PHOSPHATE:36:11143:9:122:0.99:0.80:0.23:0.35), (HYDROCORTISONE ACETATEPRAMOXINE HYDROCHLORIDE:30:11149:2:129:0.99:0.94:0.19:0.31), (CYANOCOBALAMIN CO-57FOLIC ACIDPYRIDOXINE HYDROCHLORIDE:24:11155:2:129:0.99:0.92:0.16:0.27), (NITROGEN:12:11167:4:127:0.9 9:0.75:0.09:0.15), (MAGNESIUM GLYCEROPHOSPHATE:7:11172:0:131:0.99:1.00:0.05:0.10), (LANOLINLIGHT MINERAL OILPARAFFIN:6:11173:0:131:0.99:1.00:0.04:0.08), (DOLUTEGRAVIR:100:11078:16:116: 0.99:0.86:0.46:0.60), (DAPAGLIFLOZIN PROPANEDIOLMETFORMIN HYDROCHLORIDE:84:11094:43:89:0.99:0.66:0.49:0.56), (OXYBUTYNINOXYBUTYNIN CHLORIDE:66:11112:104:28:0.99:0.39:0.70:0.50), (CRANBERRY JUICE:64:11114:7:125:0.99:0.90:0.34:0.49), (HYOSCYAMINE SULFATEMETHENAMINEMETHYLENE BLUEPHENYL SALICYLATE\SODIUM PHOSPHATE, MONOBASIC, MONOHYDRATE:63:11115:33:99:0.99:0.66:0.39:0.49), (FLUTAMIDE:61:11117:34:98:0.99: 0.64:0.38:0.48), (DIAZOXIDE:40:11138:15:117:0.99:0.73:0.25:0.38), (POLYVINYL ALCOHOL:39:11139:8:124:0.99:0.83:0.24:0.37), (NAFRONYL:15:11163:2:130:0.99:0.88:0 0.10:0.19), (CARBAZOCHROME:9:11169:1:131:0.99:0.90:0.06:0.12), (BILASTINE:99:1107 8:51:82:0.99:0.66:0.55:0.60), (ISOFLURANE:76:11101:45:88:0.99:0.63:0.46:0.53), (BIMA TOPROSTTIMOLOL:70:11107:18:115:0.99:0.80:0.38:0.51), (ZIPRASIDONE HYDROCHLORIDE:33:11144:10:123:0.99:0.77:0.21:0.33), (TRIPTORELIN:20:11157:6:127: 0.99:0.77:0.14:0.23), (DIMETHICONEHOMOSALATEOCTINOXATEOCTISALATETI TANIUM DIOXIDE:4:11173:0:133:0.99:1.00:0.03:0.06), (PHOSPHOLIPID:41:11135:4:130:0.99:0.91 0.24:0.38), (CINACALCET:40:11136:7:127:0.99:0.85:0.24:0.37), (ICATIBANT ACETATE:30:11146:3:131:0.99:0.91:0.19:0.31), (CODEINEPROMETHAZINE:29:11147:1: 133:0.99:0.97:0.18:0.30), (CABOZANTINIB:26:11150:45:89:0.99:0.37:0.23:0.28), (PARIC ALCITOL:226:10949:58:77:0.99:0.80:0.75:0.77), (BALSALAZIDE:52:11123:7:128:0.99:0.8 8:0.29:0.44), (ESTROPIPATE:49:11126:3:132:0.99:0.94:0.27:0.42), (CILAZAPRIL ANHYDROUS:34:11141:3:132:0.99:0.92:0.20:0.33), (DEXRAZOXANE:30:11145:1:134:0.9 9:0.97:0.18:0.31), (PANTHENOL:15:11160:7:128:0.99:0.68:0.10:0.18), (DEXKETOPROFE N:7:11168:0:135:0.99:1.00:0.05:0.09), (BALSALAZIDE DISODIUM:108:11066:41:95:0.99:0.72:0.53:0.61), (THIOPENTAL:71:11103:31:105:0.99:0. 70:0.40:0.51), (CLIKSTAR:57:11117:64:72:0.99:0.47:0.44:0.46), (DISOPYRAMIDE PHOSPHATE:42:11132:4:132:0.99:0.91:0.24:0.38), (NALTREXONE HYDROCHLORIDE:39:11135:7:129:0.99:0.85:0.23:0.36), (CEVIMELINE:38:11136:2:134:0 0.99:0.95:0.22:0.36), (FESOTERODINE:31:11143:13:123:0.99:0.70:0.20:0.31), (ACETAMI NOPHENBUTALBITAL:23:11151:0:136:0.99:1.00:0.14:0.25), (BENZYDAMINE:12:11162: 1:135:0.99:0.92:0.08:0.15), (LOPERAMIDE OXIDE:6:11168:0:136:0.99:1.00:0.04:0.08), (COBICISTATELVITEGRAVIREMTRICITA BINETENOFOVIR DISOPROXIL FUMARATE:75:11098:12:125:0.99:0.86:0.38:0.52), (GADODIAMIDE:60:11113:0:137:0.99: 1.00:0.30:0.47), (ASPERGILLUS NIGER ALPHA.-GALACTOSIDASE:56:11117:9:128:0.99:0.86:0.30:0.45), (TIPEPIDINE HIBENZATE:55:11118:17:120:0.99:0.76:0.31:0.45), (CEFOTIAM HYDROCHLORIDE:47:11126:9:128:0.99:0.84:0.27:0.41), (CALCIUM CHLORIDEDEXTROSE\POTASSIUM CHLORIDE\SODIUM ACETATE\SODIUM CHLORIDE:43:11130:12:125:0.99:0.78:0.26:0.39), (CARIPRAZINE:32:11141:2:135:0.99:0. 94:0.19:0.32), (INFLUENZA A VIRUS A/CALIFORNIA/7/2009 X-179A (HINI) ANTIGEN (FORMALDEHYDE INACTIVATED)\INFLUENZA A VIRUS A/VICTORIA/210/2009 X-187 (H3N2) ANTIGEN (FORMALDEHYDE INACTIVATED)\INFLUENZA B VIRUS B/BRISBANE/60/2008 ANTIGEN (FORMALDEHYDE INACTIVATED):22:11151:2:135:0.99:0.92:0.14:0.24), (CHLOROCRESOL:10:11163:1:136: 0.99:0.91:0.07:0.13), (ABACAVIR SULFATEDOLUTEGRAVIR SODIUMLAMIVUDINE:134:11038:25:113:0.99:0.84:0.54:0.66), (IRONPOLYSACCHARI DES:75:11097:29:109:0.99:0.72:0.41:0.52), (PERAMPANEL:69:11103:18:120:0.99:0.79:0.3 7:0.50), (CONVALLARIA MAJALISCRATAEGUS LAEVIGATA LEAFPROXYPHYLLINE:68:11104:9:129:0.99:0.88:0.35:0.50), (EPHEDRINE HYDROCHLORIDE:57:11115:20:118:0.99:0.74:0.33:0.45), (AZELNIDIPINEOLMESART AN MEDOXOMIL:55:11117:4:134:0.99:0.93:0.29:0.44), (ASCORBIC ACIDBIOTINCYANOCOBALAMINFOLIC ACIDNIACINPANTOTHENIC ACIDPYRIDOXINE\RIBOFLAVINTHIAMINE:53:11119:36:102:0.99:0.60:0.34:0.43), (DE QUALINIUM CHLORIDE:40:11132:7:131:0.99:0.85:0.23:0.37), (MENTHOLTROLAMINE SALICYLATE:30:11142:0:138:0.99:1.00:0.18:0.30), (GADOTERATE MEGLUMINE:26:11146:13:125:0.99:0.67:0.17:0.27), (SARGRAMOSTIM:22:11150:10:128: 0.99:0.69:0.15:0.24), (OXETHAZAINE:20:11152:2:136:0.99:0.91:0.13:0.22), (ACETYLLE UCINE:17:11155:0:138:0.99:1.00:0.11:0.20), (POLATUZUMAB VEDOTIN:5:11167:0:138:0.99:1.00:0.03:0.07), (ACETAMINOPHENPROPOXYPHENE NAPSYLATE:123:11048:73:66:0.99:0.63:0.65:0.64), (YEAST:85:11086:52:87:0.99:0.62:0.4 9:0.55), (AMLODIPINE BESYLATEIRBESARTAN:81:11090:21:118:0.99:0.79:0.41:0.54), (ANHYDROUS CITRIC ACIDASPIRIN\SODIUM BICARBONATE:79:11092:21:118:0.99:0.79:0.40:0.53), (ASCORBIC ACIDBIOTINCALCIUM PANTOTHENATECOBALAMINFOLIC ACIDNIACINAMIDEPYRIDOXINE HYDROCHLORIDE\RIBOFLAVINTHIAMINE MONONITRATE:73:11098:38:101:0.99:0.66:0.42:0.51), (DIPHENIDOL HYDROCHLORIDE:64:11107:26:113:0.99:0.71:0.36:0.48), (BECLOMETHASONE DIPROPIONATEFORMOTEROL:62:11109:34:105:0.99:0.65:0.37:0.47), (BETAMETHASO NE BUTYRATE PROPIONATE:57:11114:17:122:0.99:0.77:0.32:0.45), (.ALPHA.-TOCOPHEROLRETINOL:43:11128:14:125:0.99:0.75:0.26:0.38), (CHLORMADINONE ACETATE:39:11132:14:125:0.99:0.74:0.24:0.36), (FLUOCINOLONE:37:11134:5:134:0.99: 0.88:0.22:0.35), (CLOXACILLIN SODIUM:36:11135:27:112:0.99:0.57:0.24:0.34), (PRILOCAINE:21:11150:2:137:0.99:0.91:0 0.13:0.23), (CHORIOGONADOTROPIN ALFA:18:11153:0:139:0.99:1.00:0.11:0.21), (.ALPHA.-TOCOPHEROL ACETATE:17:11154:0:139:0.99:1.00:0.11:0.20), (BILBERRY:16:11155:2:137:0.99:0.89:0.1 0:0.19), (TETRACYCLINE HYDROCHLORIDE:5:11166:0:139:0.99:1.00:0.03:0.07), (CYCLOSPORINE:2067:9103:0:14 0:0.99:1.00:0.94:0.97), (ATOMOXETINE HYDROCHLORIDE:347:10823:0:140:0.99:1.00:0.71:0.83), (ASPARAGINASE:106:11064:2 7:113:0.99:0.80:0.48:0.60), (AMEZINIUM METILSULFATE:87:11083:14:126:0.99:0.86:0.41:0.55), (ANAGRELIDE HYDROCHLORIDE:86:11084:42:98:0.99:0.67:0.47:0.55), (ACETAMINOPHENDICHLOR ALPHENAZONEISOMETHEPTENE:85:11085:15:125:0.99:0.85:0.40:0.55), (BRINZOLAM IDETIMOLOL MALEATE:73:11097:26:114:0.99:0.74:0.39:0.51), (ETOPOSIDE PHOSPHATE:57:11113:36:104:0.99:0.61:0.35:0.45), (FEXOFENADINE HYDROCHLORIDEPSEUDOEPHEDRINE HYDROCHLORIDE:29:11141:9:131:0.99:0.76:0.18:0.29), (SAXAGLIPTIN:26:11144:1:139: 0.99:0.96:0.16:0.27), (BACITRACIN ZINCPOLYMYXIN B SULFATE:25:11145:2:138:0.99:0.93:0.15:0.26), (GLYCYRRHIZIN:15:11155:2:138:0.99:0.8 8:0.10:0.18), (FOSFOMYCIN TROMETHAMINE:9:11161:2:138:0.99:0.82:0.06:0.11), (FERUMOXYTOL NON-STOICHIOMETRIC MAGNETITE:7:11163:0:140:0.99:1.00:0.05:0.09), (NEVIRAPINE:167:11002:12:129:0.99:0. 93:0.56:0.70), (ALDESLEUKIN:60:11109:62:79:0.99:0.49:0.43:0.46), (FUDOSTEINE:50:1 1119:9:132:0.99:0.85:0.27:0.41), (DEHYDROEPIANDROSTERONE-3-SULFATE SODIUM:36:11133:3:138:0.99:0.92:0.21:0.34), (ENALAPRILHYDROCHLOROTHIAZIDE: 32:11137:1:140:0.99:0.97:0.19:0.31), (MAGNESIUM GLYCINATE:21:11148:2:139:0.99:0.91:0.13:0.23), (BISMUTH SUBCITRATERANITIDINE HYDROCHLORIDESUCRALFATE:14:11155:2:139:0.99:0.88:0.09:0.17), (BUTENAFINE HYDROCHLORIDE:10:11159:0:141:0.99:1.00:0.07:0.12), (KARAYA GUM:9:11160:0:141:0.99:1.00:0.06:0.11), (CARBOHYDRATESFATTY ACIDSMINERALSPROTEINVITAMINS:50:11118:11:131:0.99:0.82:0.28:0.41), (COAGU LATION FACTOR VIIA RECOMBINANT HUMAN:40:11128:6:136:0.99:0.87:0.23:0.36), (VORTIOXETINE:22:11146:0:142:0.99:1.00: 0.13:0.24), (AMINO ACIDSDEXTROSESOYBEAN OIL:5:11163:0:142:0.99:1.00:0.03:0.07), (SELEGILINE:137:11030:44:99:0.99:0.76:0.58:0. 66), (DESFLURANE:112:11055:40:103:0.99:0.74:0.52:0.61), (NORETHINDRONE ACETATE:97:11070:4:139:0.99:0.96:0.41:0.58), (CYANOCOBALAMINFOLIC ACIDPYRIDOXINE:83:11084:11:132:0.99:0.88:0.39:0.54), (ASPIRINCLOPIDOGREL:82: 11085:40:103:0.99:0.67:0.44:0.53), (POTASSIUM PHOSPHATE, MONOBASIC:44:11123:20:123:0.99:0.69:0.26:0.38), (LANDIOLOL HYDROCHLORIDE:36:11131:25:118:0.99:0.59:0.23:0.33), (PARAFFIN:17:11150:1:142:0.9 9:0.94:0.11:0.19), (DORZOLAMIDETIMOLOL:10:11157:0:143:0.99:1.00:0.07:0.12), (LO XAPINE:10:11157:2:141:0.99:0.83:0.07:0.12), (TAPENTADOL:9:11158:0:143:0.99:1.00:0. 06:0.11), (GOSERELIN ACETATE:326:10840:18:126:0.99:0.95:0.72:0.82), (PHENTERMINE HYDROCHLORIDE:102:11064:12:132:0.99:0.89:0.44:0.59), (SORBITOL:73:11093:31:113: 0.99:0.70:0.39:0.50), (HYDROCHLOROTHIAZIDEMETOPROLOL SUCCINATE:69:11097:20:124:0.99:0.78:0.36:0.49), (ACECLOFENAC:55:11111:19:125:0.9 9:0.74:0.31:0.43), (IRON POLYMALTOSE:43:11123:16:128:0.99:0.73:0.25:0.37), (CREATINE:8:11158:0:144:0.99:1. 00:0.05:0.10), (IOMEPROL:8:11158:0:144:0.99:1.00:0.05:0.10), (ADEMETIONINE:6:1116 0:0:144:0.99:1.00:0.04:0.08), (EMTRICITABINETENOFOVIR DISOPROXIL FUMARATE:605:10560:0:145:0.99:1.00:0.81:0.89), (LEVOTHYROXINE SODIUMPOTASSIUM IODIDE:96:11069:44:101:0.99:0.69:0.49:0.57), (BISMUTH SUBSALICYLATECALCIUM CARBONATE:78:11087:4:141:0.99:0.95:0.36:0.52), (UROKINASE:56:11109:30:115:0.99:0. 65:0.33:0.44), (MAGALDRATE:49:11116:6:139:0.99:0.89:0.26:0.40), (PENTOSTATIN:35: 11130:109:36:0.99:0.24:0.49:0.33), (ADAPALENE:34:11131:2:143:0.99:0.94:0.19:0.32), (S ENNOSIDES A AND B CALCIUM:21:11144:1:144:0.99:0.95:0.13:0.22), (SODIUM CHONDROITIN SULFATE:20:11145:2:143:0.99:0.91:0.12:0.22), (LEVOSULPIRIDE:9:11156:0:145:0.99:1.0 0:0.06:0.11), (MARAVIROC:95:11069:9:137:0.99:0.91:0.41:0.57), (LACTOBACILLUS ACIDOPHILUS\LACTOBACILLUS DELBRUECKII SUBSP. BULGARICUS:91:11073:24:122:0.99:0.79:0.43:0.55), (BOCOCIZUMAB:74:11090:27:119:0 0.99:0.73:0.38:0.50), (HYDROTALCITE:60:11104:0:146:0.99:1.00:0.29:0.45), (ASCORBIC ACIDBIOTINCALCIUM PANTOTHENATECYANOCOBALAMINFOLIC ACIDNIACINAMIDEPYRIDOXINE HYDROCHLORIDE\RIBOFLAVINTHIAMINE MONONITRATE:57:11107:12:134:0.99:0.83:0.30:0.44), (TERCONAZOLE:56:11108:8:138: 0.99:0.88:0.29:0.43), (BEPOTASTINE BESILATE:46:11118:10:136:0.99:0.82:0.25:0.39), (FLURAZEPAM HYDROCHLORIDE:44:11120:5:141:0.99:0.90:0.24:0.38), (NAFAMOSTAT MESYLATE:39:11125:10:136:0.99:0.80:0.22:0.35), (QUINIDINE:10:11154:0:146:0.99:1.00: 0.06:0.12), (TROPISETRON:4:11160:0:146:0.99:1.00:0.03:0.05), (AMOXICILLINCLARIT HROMYCINESOMEPRAZOLE:72:11091:63:84:0.99:0.53:0.46:0.49), (ESTROGENS, ESTERIFIEDMETHYLTESTOSTERONE:65:11098:7:140:0.99:0.90:0.32:0.47), (ANIDULA FUNGIN:63:11100:23:124:0.99:0.73:0.34:0.46), (TIBOLONE:58:11105:31:116:0.99:0.65:0. 33:0.44), (PANCRELIPASEPANCRELIPASE AMYLASEPANCRELIPASE LIPASEPANCRELIPASE PROTEASE:48:11115:7:140:0.99:0.87:0.26:0.40), (TESTOSTERONE ENANTHATE:46:11117:11:136:0.99:0.81:0.25:0.38), (CARBASALATE:44:11119:7:140:0.9 9:0.86:0.24:0.37), (DOCOSANOL:31:11132:2:145:0.99:0.94:0.18:0.30), (ACALABRUTINI B:4:11159:0:147:0.99:1.00:0.03:0.05), (BEMIPARIN SODIUM:4:11159:0:147:0.99:1.00:0.03:0.05), (AMBRISENTAN:700:10462:0:148:0.99:1.00: 0.83:0.90), (MILRINONE:130:11032:9:139:0.99:0.94:0.48:0.64), (BETAMETHASONE DIPROPIONATECALCIPOTRIENECALCIPOTRIENE HYDRATE:116:11046:97:51:0.99:0.54:0.69:0.61), (ROSIGLITAZONE MALEATE:112:11050:19:129:0.99:0.85:0.46:0.60), (TICLOPIDINE:96:11066:34:114:0.99:0 0.74:0.46:0.56), (CISATRACURIUM:86:11076:21:127:0.99:0.80:0.40:0.54), (ISONIAZIDRI FAMPIN:81:11081:19:129:0.99:0.81:0.39:0.52), (ROFECOXIB:77:11085:33:115:0.99:0.70: 0.40:0.51), (MAXACALCITOL:74:11088:23:125:0.99:0.76:0.37:0.50), (FERROUS GLYCINE SULFATE:64:11098:22:126:0.99:0.74:0.34:0.46), (TIANEPTINE:46:11116:8:140:0.99:0.85 0.25:0.38), (NAPHAZOLINE HYDROCHLORIDEPHENIRAMINE MALEATE:20:11142:2:146:0.99:0.91:0.12:0.21), (RIBOCICLIB:18:11144:5:143:0.99:0.78: 0.11:0.20), (POTASSIUM PHOSPHATE, DIBASIC:6:11156:0:148:0.99:1.00:0.04:0.08), (PYRAZINAMIDE:419:10742:117:32:0.99:0. 78:0.93:0.85), (ORPHENADRINE:124:11037:59:90:0.99:0.68:0.58:0.62), (METFORMINSI TAGLIPTIN:61:11100:17:132:0.99:0.78:0.32:0.45), (ST. JOHN'S WORT:48:11113:8:141:0.99:0.86:0.25:0.39), (BROMHEXINE:47:11114:17:132:0.99:0.73:0. 26:0.39), (CYANOCOBALAMINFOLIC ACIDPYRIDOXINE HYDROCHLORIDE:27:11134:1:148:0.99:0.96:0.15:0.27), (MEQUITAZINE:21:11140:3:146: 0.99:0.88:0.13:0.22), (DISULFIRAM:20:11141:0:149:0.99:1.00:0.12:0.21), (BENZODIAZ EPINE:18:11143:0:149:0.99:1.00:0.11:0.19), (TROPATEPINE HYDROCHLORIDE:15:11146:0:149:0.99:1.00:0.09:0.17), (FOLATE SODIUM:359:10801:9:141:0.99:0.98:0.72:0.83), (GATIFLOXACIN:154:11006:0:150:0.99:1. 00:0.51:0.67), (CALCIUM CARBONATEERGOCALCIFEROLRETINOL:89:11071:65:85:0.99:0.58:0.51:0.54), (TRIM IPRAMINE MALEATE:81:11079:81:69:0.99:0.50:0.54:0.52), (ERIBULIN MESYLATE:66:11094:27:123:0.99:0.71:0.35:0.47), (METHENAMINE:61:11099:10:140:0.9 9:0.86:0.30:0.45), (PINDOLOL:56:11104:14:136:0.99:0.80:0.29:0.43), (BENAZEPRIL HYDROCHLORIDEHYDROCHLOROTHIAZIDE:52:11108:1:149:0.99:0.98:0.26:0.41), (B ETAMETHASONECLOTRIMAZOLE:41:11119:1:149:0.99:0.98:0.22:0.35), (FAMOTIDIN EIBUPROFEN:41:11119:1:149:0.99:0.98:0.22:0.35), (TEA LEAF:38:11122:1:149:0.99:0.97:0.20:0.34), (DOTHIEPIN HYDROCHLORIDE:16:11144:0:150:0.99:1.00:0.10:0.18), (CITRIC ACID MONOHYDRATE:14:11146:1:149:0.99:0.93:0.09:0.16), (DIPHENHYDRAMINE HYDROCHLORIDENAPROXEN SODIUM:9:11151:0:150:0.99:1.00:0.06:0.11), (MEXILETINE:82:11077:23:128:0.99:0.78:0. 39:0.52), (DICLOXACILLIN:74:11085:10:141:0.99:0.88:0.34:0.49), (TROSPIUM:69:11090: 15:136:0.99:0.82:0.34:0.48), (CLOFAZIMINE:61:11098:17:134:0.99:0.78:0.31:0.45), (CAL CIUMVITAMIN DVITAMIN K5:52:11107:2:149:0.99:0.96:0.26:0.41), (ECALLANTIDE:48:11111:16:135:0.99:0.75:0.26: 0.39), (KELP:24:11135:0:151:0.99:1.00:0.14:0.24), (CINNARIZINE:16:11143:0:151:0.99:1. 00:0.10:0.17), (BRIMONIDINEBRIMONIDINE TARTRATE:15:11144:0:151:0.99:1.00:0.09:0.17), (DESVENLAFAXINE:86:11072:17:135:0. 99:0.83:0.39:0.53), (MAGNESIUM ASPARTATE\POTASSIUM ASPARTATE:66:11092:43:109:0.99:0.61:0.38:0.46), (LANREOTIDE:48:11110:72:80:0.99:0 0.40:0.38:0.39), (DEXTROSE\POTASSIUM CHLORIDE\SODIUM CHLORIDE\SODIUM LACTATE:33:11125:5:147:0.99:0.87:0.18:0.30), (CLOMETHIAZOLE:23:11135:8:144:0.99: 0.74:0.14:0.23), (HUMAN BLOOD CELLS:16:11142:4:148:0.99:0.80:0.10:0.17), (LATANOPROSTTIMOLOL MALEATE:13:11145:0:152:0.99:1.00:0.08:0.15), (IOVERSOL:4:11154:0:152:0.99:1.00:0.0 3:0.05), (MITOXANTRONE HYDROCHLORIDE:205:10952:19:134:0.99:0.92:0.60:0.73), (TAURINE:40:11117:8:145:0.9 9:0.83:0.22:0.34), (PHOSPHORUS:36:11121:5:148:0.99:0.88:0.20:0.32), (POLYMYXIN B:19:11138:2:151:0.99:0.90:0.11:0.20), (EFAVIRENZEMTRICITABINETENOFOVIR DISOPROXIL FUMARATE:184:10972:7:147:0.99:0.96:0.56:0.70), (CHLORPHENIRAMINEHYDROCOD ONE:149:11007:49:105:0.99:0.75:0.59:0.66), (TAFLUPROST:102:11054:29:125:0.99:0.78:0 0.45:0.57), (FERROUS SULFATEGLYCINE:98:11058:96:58:0.99:0.51:0.63:0.56), (PRASUGREL:77:11079:10:144: 0.99:0.89:0.35:0.50), (TEDUGLUTIDE\WATER:62:11094:100:54:0.99:0.38:0.53:0.45), (T RIPTORELIN PAMOATE:53:11103:77:77:0.99:0.41:0.41:0.41), (HALOPERIDOL DECANOATE:52:11104:25:129:0.99:0.68:0.29:0.40), (NABILONE:41:11115:6:148:0.99:0.8 7:0.22:0.35), (VIDARABINE:41:11115:3:151:0.99:0.93:0.21:0.35), (BENZOYL PEROXIDECLINDAMYCIN PHOSPHATE:39:11117:3:151:0.99:0.93:0.21:0.34), (TRIMETHOBENZAMIDE HYDROCHLORIDE:37:11119:0:154:0.99:1.00:0.19:0.32), (AMYLASELIPASEPROTEAS E:26:11130:1:153:0.99:0.96:0.15:0.25), (ESLICARBAZEPINE ACETATE:14:11142:1:153:0.99:0.93:0.08:0.15), (DEXKETOPROFEN TROMETAMOL:11:11145:0:154:0.99:1.00:0.07:0.13), (ITOPRIDE:50:11105:13:142:0.99:0. 79:0.26:0.39), (DARIFENACINDARIFENACIN HYDROBROMIDE:31:11124:1:154:0.99:0.97:0.17:0.29), (SODIUM FERRIC GLUCONATE COMPLEX:19:11136:1:154:0.99:0.95:0.11:0.20), (CYANOCOBALAMINPYRIDOXAMINE HYDROCHLORIDETHIAMINE HYDROCHLORIDE:13:11142:2:153:0.99:0.87:0.08:0.14), (FOSPHENYTOINFOSPHENYT OIN SODIUM:106:11048:61:95:0.99:0.63:0.53:0.58), (SODIUM CITRATE:63:11091:73:83:0.99:0.46:0.43:0.45), (DORIPENEM:58:11096:10:146:0.99:0.85: 0.28:0.43), (MENAQUINONE 6:50:11104:3:153:0.99:0.94:0.25:0.39), (SODIUM GUALENATE MONOHYDRATE:41:11113:19:137:0.99:0.68:0.23:0.34), (ALTHIAZIDESPIRONOLACTO NE:40:11114:2:154:0.99:0.95:0.21:0.34), (ALUMINUM HYDROXIDEMAGNESIUM HYDROXIDE:13:11141:0:156:0.99:1.00:0.08:0.14), (CALCIUM PHOSPHATE, DIBASIC, ANHYDROUSMAGNESIUM GLYCEROPHOSPHATEPHOSPHORIC ACIDSODIUM PHOSPHATE, DIBASIC:7:11147:0:156:0.99:1.00:0.04:0.08), (LANTHANUM CARBONATE:204:10949:1:156:0.99:1.00:0.57:0.72), (LEVOMILNACIPRAN HYDROCHLORIDE:107:11046:23:134:0.99:0.82:0.44:0.58), (AMBROXOLCLENBUTERO L:74:11079:19:138:0.99:0.80:0.35:0.49), (DISOPYRAMIDE:63:11090:28:129:0.99:0.69:0.3 3:0.45), (VANDETANIB:46:11107:25:132:0.99:0.65:0.26:0.37), (FOSFLUCONAZOLE:43:1 1110:14:143:0.99:0.75:0.23:0.35), (DIMETHICONE 410:39:11114:9:148:0.99:0.81:0.21:0.33), (MAGNESIUM ISOGLYCYRRHIZINATE:36:11117:1:156:0.99:0.97:0.19:0.31), (ASCORBIC ACIDCALCIUM PANTOTHENATECYANOCOBALAMINNIACINAMIDEPYRIDOXINE HYDROCHLORIDE\RIBOFLAVINTHIAMINE MONONITRATE:24:11129:1:156:0.99:0.96:0.13:0.23), (ALVERINE CITRATEDIMETHICONE:9:11144:0:157:0.99:1.00:0.05:0.10), (ESMOLOL HYDROCHLORIDE:80:11072:57:101:0.99:0.58:0.44:0.50), (ASIAN GINSENG:64:11088:11:147:0.99:0.85:0.30:0.45), (POTASSIUM IODIDE:62:11090:28:130:0.99:0.69:0.32:0.44), (CLOXACILLIN:21:11131:0:158:0.99:1.00: 0.12:0.21), (CYCLOSERINE:7:11145:0:158:0.99:1.00:0.04:0.08), (SUFENTANIL CITRATE:111:11040:43:116:0.99:0.72:0.49:0.58), (CABAZITAXEL:93:11058:49:110:0.99: 0.65:0.46:0.54), (BOCEPREVIR:86:11065:38:121:0.99:0.69:0.42:0.52), (ITRACONAZOLE SODIUM CHLORIDE:60:11091:31:128:0.99:0.66:0.32:0.43), (IBRITUMOMAB TIUXETAN:50:11101:131:28:0.99:0.28:0.64:0.39), (CODEINE SULFATE:37:11114:6:153:0.99:0.86:0.19:0.32), (ULINASTATIN:26:11125:0:159:0.99:1.00: 0.14:0.25), (AVOBENZONEOCTOCRYLENETRICLOSAN:7:11144:0:159:0.99:1.00:0.04: 0.08), (DOXORUBICIN HYDROCHLORIDE:959:10191:7:153:0.99:0.99:0.86:0.92), (NEOSTIGMINE:102:11048:30: 130:0.99:0.77:0.44:0.56), (SUGAMMADEX:89:11061:17:143:0.99:0.84:0.38:0.53), (ZILEU TON:75:11075:114:46:0.99:0.40:0.62:0.48), (IDARUCIZUMAB:61:11089:50:110:0.99:0.55: 0.36:0.43), (TRANDOLAPRILVERAPAMIL HYDROCHLORIDE:31:11119:0:160:0.99:1.00:0.16:0.28), (ETHINYL ESTRADIOLLEVONORGESTREL:442:10707:0:161:0.99:1.00:0.73:0.85), (POTASSIUM CITRATETRISODIUM CITRATE DIHYDRATE:66:11083:47:114:0.99:0.58:0.37:0.45), (PERAMIVIR:31:11118:3:158:0.99:0.9 1:0.16:0.28), (NEUROTROPIN:30:11119:2:159:0.99:0.94:0.16:0.27), (QUERCETIN:30:111 19:3:158:0.99:0.91:0.16:0.27), (LIPEGFILGRASTIM:7:11142:0:161:0.99:1.00:0.04:0.08), (ACETAMINOPHEN\CAFFEINE:107:11041:51:111:0.99:0.68:0.49:0.57), (SUCCINYLCHO LINE CHLORIDE:100:11048:27:135:0.99:0.79:0.43:0.55), (CIDOFOVIR:57:11091:1:161:0.99:0.9 8:0.26:0.41), (GLYBURIDEMETFORMIN:35:11113:0:162:0.99:1.00:0.18:0.30), (PIPAMP ERONE:26:11122:2:160:0.99:0.93:0.14:0.24), (PROTEIN:19:11129:2:160:0.99:0.90:0.11:0. 19), (CLOXAZOLAM:87:11060:18:145:0.99:0.83:0.38:0.52), (CHONDROITIN SULFATE (CHICKEN):70:11077:31:132:0.99:0.69:0.35:0.46), (ERIBULIN:66:11081:89:74:0.99:0.43:0 0.47:0.45), (CLOFARABINE:58:11089:16:147:0.99:0.78:0.28:0.42), (CODEINE PHOSPHATEPROMETHAZINE HYDROCHLORIDE:30:11117:3:160:0.99:0.91:0.16:0.27), (DIACETYLMORPHINE:27:111 20:1:162:0.99:0.96:0.14:0.25), (COAGULATION FACTOR IX HUMANCOAGULATION FACTOR VII HUMANCOAGULATION FACTOR X HUMANPROTEIN CPROTEIN S HUMANPROTHROMBIN:20:11127:9:154:0.99:0.69:0.11:0.20), (HYDROXYETHYL STARCH 130/0.4:6:11141:0:163:0.99:1.00:0.04:0.07), (PHENAZOPYRIDINE HYDROCHLORIDE:96:11050:18:146:0.99:0.84:0.40:0.54), (BETAMETHASONE DIPROPIONATECALCIPOTRIENE:84:11062:47:117:0.99:0.64:0.42:0.51), (ETRETINATE: 37:11109:131:33:0.99:0.22:0.53:0.31), (SELENIUM SULFIDE:28:11118:3:161:0.99:0.90:0.15:0.25), (NALBUPHINE:8:11138:2:162:0.99:0.80:0. 05:0.09), (ENTACAPONE:131:11014:7:158:0.99:0.95:0.45:0.61), (CALCIUMVITAMINS:6 2:11083:12:153:0.99:0.84:0.29:0.43), (HALOBETASOL PROPIONATE:50:11095:4:161:0.99:0.93:0.24:0.38), (INTERFERON ALFA-2A OR INTERFERON ALFA-2B:38:11107:31:134:0.99:0.55:0.22:0.32), (INGENOL MEBUTATE:22:11123:0:165:0.99:1.00:0.12:0.21), (MIZORIBINE:184:10960:27:139:0.99:0. 87:0.57:0.69), (MOLSIDOMINE:58:11086:5:161:0.99:0.92:0.26:0.41), (ASPIRINCAFFEIN E:51:11093:2:164:0.99:0.96:0.24:0.38), (FOSPHENYTOIN SODIUM:51:11093:8:158:0.99:0.86:0.24:0.38), (ABOBOTULINUMTOXINA:48:11096:77:8 9:0.99:0.38:0.35:0.37), (ZINC GLUCONATE:47:11097:1:165:0.99:0.98:0.22:0.36), (BETAMETHASONEDEXCHLORPH ENIRAMINE MALEATE:38:11106:5:161:0.99:0.88:0.19:0.31), (VINPOCETINE:29:11115:5:161:0.99:0.85: 0.15:0.26), (SALSALATE:28:11116:2:164:0.99:0.93:0.15:0.25), (IBUPROFENPSEUDOEP HEDRINE HYDROCHLORIDE:9:11135:0:166:0.99:1.00:0.05:0.10), (HUMAN C1-ESTERASE INHIBITOR:71:11072:8:159:0.99:0.90:0.31:0.46), (CISAPRIDE:50:11093:14:153:0.99:0.78: 0.25:0.37), (FUROSEMIDESPIRONOLACTONE:47:11096:7:160:0.99:0.87:0.23:0.36), (BR OMELAINS:31:11112:1:166:0.99:0.97:0.16:0.27), (ALCLOMETASONE DIPROPIONATE:20:11123:1:166:0.99:0.95:0.11:0.19), (TUBERCULIN PURIFIED PROTEIN DERIVATIVE:6:11137:0:167:0.99:1.00:0.03:0.07), (MEXILETINE HYDROCHLORIDE:103:11039:21:147:0.99:0.83:0.41:0.55), (IVABRADINE HYDROCHLORIDE:97:11045:23:145:0.99:0.81:0.40:0.54), (TRABECTEDIN:67:11075:21:1 47:0.99:0.76:0.31:0.44), (PROPATYL NITRATE:53:11089:15:153:0.99:0.78:0.26:0.39), (THIOCOLCHICOSIDE:8:11134:0:168:0.9 9:1.00:0.05:0.09), (DEMCIZUMAB:6:11136:0:168:0.99:1.00:0.03:0.07), (LACIDIPINE:60: 11081:6:163:0.99:0.91:0.27:0.42), (DIMEMORFAN:59:11082:5:164:0.99:0.92:0.26:0.41), (ETHINYL ESTRADIOL:11:11130:6:163:0.99:0.65:0.06:0.12), (ANAKINRA:99:11041:24:146:0.98:0.80: 0.40:0.54), (CERTOPARIN SODIUM:73:11067:30:140:0.98:0.71:0.34:0.46), (FLOMOXEF SODIUM:30:11110:5:165:0.98:0.86:0.15:0.26), (LACTITOL:30:11110:6:164:0.98:0.83:0.15: 0.26), (AMLODIPINE BESYLATEHYDROCHLOROTHIAZIDEOLMESARTAN MEDOXOMIL:187:10952:92:79:0.98:0.67:0.70:0.69), (DUTASTERIDETAMSULOSIN HYDROCHLORIDE:154:10985:87:84:0.98:0.64:0.65:0.64), (MAGNESIUM ASPARTATE HYDROCHLORIDE:94:11045:46:125:0.98:0.67:0.43:0.52), (TENELIGLIPTIN:77:11062:10: 161:0.98:0.89:0.32:0.47), (COLLAGEN:69:11070:9:162:0.98:0.88:0.30:0.45), (ACETYLCA RNITINE HYDROCHLORIDE:54:11085:9:162:0.98:0.86:0.25:0.39), (CLOBETASONE BUTYRATE:39:11100:3:168:0.98:0.93:0.19:0.31), (GABEXATE MESYLATE:39:11100:11:160:0.98:0.78:0.20:0.31), (INTERFERON GAMMA-1B:34:11105:130:41:0.98:0.21:0.45:0.28), (EGG PHOSPHOLIPIDSSAFFLOWER OILSOYBEAN OIL:19:11120:0:171:0.98:1.00:0.10:0.18), (ALIZAPRIDE:13:11126:0:171:0.98:1.00:0.07:0. 13), (BETAHISTINE MESILATE:91:11047:19:153:0.98:0.83:0.37:0.51), (BUTALBITAL:89:11049:11:161:0.98:0. 89:0.36:0.51), (SOFOSBUVIRVELPATASVIR:88:11050:57:115:0.98:0.61:0.43:0.51), (RO PIVACAINE HYDROCHLORIDE:72:11066:15:157:0.98:0.83:0.31:0.46), (POTASSIUM BICARBONATE\POTASSIUM CHLORIDE:10:11128:1:171:0.98:0.91:0.06:0.10), (PERPHENAZINE:81:11056:19:154:0.98: 0.81:0.34:0.48), (POTASSIUM PHOSPHATE, MONOBASIC\SODIUM PHOSPHATE, DIBASIC, ANHYDROUS\SODIUM PHOSPHATE, MONOBASIC, MONOHYDRATE:40:11097:5:168:0.98:0.89:0.19:0.32), (ETHYL ICOSAPENTATE:21:11116:1:172:0.98:0.95:0.11:0.20), (PREDNICARBATE:7:11130:0:173: 0.98:1.00:0.04:0.07), (DEGARELIX:87:11049:54:120:0.98:0.62:0.42:0.50), (ALBUMIN BOVINETANNIC ACID:64:11072:7:167:0.98:0.90:0.28:0.42), (ATROPINE SULFATEHYOSCYAMINE SULFATEPHENOBARBITALSCOPOLAMINE HYDROBROMIDE:59:11077:8:166:0.98:0.88:0.26:0.40), (DIPHENOXYLATE:40:11096:0:1 74:0.98:1.00:0.19:0.31), (DEXAMETHASONENEOMYCIN SULFATEPOLYMYXIN B SULFATE:22:11114:1:173:0.98:0.96:0.11:0.20), (ACETAMINOPHENDIHYDROCODEINE: 18:11118:0:174:0.98:1.00:0.09:0.17), (CAPSAICIN:137:10998:44:131:0.98:0.76:0.51:0.61), (CHLOROTHIAZIDE:115:11020:46:129:0.98:0.71:0.47:0.57), (OXYMORPHONE:112:1102 3:26:149:0.98:0.81:0.43:0.56), (HYDROCHLOROTHIAZIDESPIRONOLACTONE:100:110 35:11:164:0.98:0.90:0.38:0.53), (ETHINYL ESTRADIOLNORGESTREL:89:11046:56:119:0.98:0.61:0.43:0.50), (PALIVIZUMAB:87:11 048:1:174:0.98:0.99:0.33:0.50), (CAMOSTAT MESYLATE:58:11077:5:170:0.98:0.92:0.25:0.40), (CROTAMITON:58:11077:8:167:0.98:0. 88:0.26:0.40), (GLYCOPYRROLATEINDACATEROL MALEATE:52:11083:127:48:0.98:0.29:0.52:0.37), (MANIDIPINE:41:11094:1:174:0.98:0.98: 0.19:0.32), (COSMETICS:5:11130:0:175:0.98:1.00:0.03:0.05), (THEOPHYLLINE:89:11045: 75:101:0.98:0.54:0.47:0.50), (ADENOSINE TRIPHOSPHATE:57:11077:8:168:0.98:0.88:0.25:0.39), (MEBEVERINE HYDROCHLORIDE:11:11123:0:176:0.98:1.00:0.06:0.11), (AMMONIUM GLYCYRRHIZATEGLYCINEMETHIONINE:67:11066:7:170:0.98:0.91:0.28:0.43), (EVEN ING PRIMROSE OIL:66:11067:4:173:0.98:0.94:0.28:0.43), (MENTHOLMETHYL SALICYLATE:46:11087:1:176:0.98:0.98:0.21:0.34), (NICERGOLINE:31:11102:5:172:0.98: 0.86:0.15:0.26), (ASCORBIC ACIDPOLYETHYLENE GLYCOL 3350POTASSIUM CHLORIDE\SODIUM ASCORBATE\SODIUM CHLORIDE\SODIUM SULFATE:26:11107:6:171:0.98:0.81:0.13:0.23), (PLANTAGO OVATA SEED:18:11115:0:177:0.98:1.00:0.09:0.17), (ETHINYL ESTRADIOLNORETHINDRONE:132:11000:17:161:0.98:0.89:0.45:0.60), (UBIQUINOL:64: 11068:13:165:0.98:0.83:0.28:0.42), (ENSULIZOLEEUCERINOCTINOXATEOCTISALA TETITANIUM DIOXIDE\ZINC OXIDE:53:11079:7:171:0.98:0.88:0.24:0.37), (CEFOZOPRAN HYDROCHLORIDE:38:11094:4:174:0.98:0.90:0.18:0.30), (HYDROCODONE BITARTRATEIBUPROFEN:131:11000:87:92:0.98:0.60:0.59:0.59), (PILSICAINIDE HYDROCHLORIDE:118:11013:55:124:0.98:0.68:0.49:0.57), (INFLIXIMAB-DYYB:81:11050:122:57:0.98:0.40:0.59:0.48), (PANOBINOSTAT:43:11088:3:176:0.98:0.93: 0.20:0.32), (BETAHISTINE HYDROCHLORIDE:15:11116:0:179:0.98:1.00:0.08:0.14), (METILDIGOXIN:127:11003:22: 158:0.98:0.85:0.45:0.59), (METFORMIN PAMOATE:70:11060:29:151:0.98:0.71:0.32:0.44), (NEOMYCIN:70:11060:25:155:0.98:0.74: 0.31:0.44), (KETOTIFEN:43:11087:7:173:0.98:0.86:0.20:0.32), (METHYLNALTREXONE BROMIDE:43:11087:3:177:0.98:0.93:0.20:0.32), (NIMODIPINE:40:11090:6:174:0.98:0.87: 0.19:0.31), (ETHAMBUTOL HYDROCHLORIDE:509:10620:28:153:0.98:0.95:0.77:0.85), (ALBIGLUTIDE:119:11010:99: 82:0.98:0.55:0.59:0.57), (TRIHEXYPHENIDYL:96:11033:42:139:0.98:0.70:0.41:0.51), (C ALCIUM GLYCEROPHOSPHATEPOLYETHYLENE GLYCOLSPOWDERED CELLULOSESACCHARIN SODIUM DIHYDRATE\SODIUM HYDROXIDE\SODIUM LAURYL SULFATE\SODIUM MAGNESIUM SILICATE\SODIUM MONOFLUOROPHOSPHATESORBITOLWATER:42:11087:2:179:0.98:0.95:0.19:0.32), (BRIMONIDINE TARTRATEBRINZOLAMIDE:41:11088:1:180:0.98:0.98:0.19:0.31), (BENZOYL PEROXIDE:24:11105:0:181:0.98:1.00:0.12:0.21), (DAPAGLIFLOZIN:341:10787:13:169:0.9 8:0.96:0.67:0.79), (INOTUZUMAB OZOGAMICIN:99:11029:38:144:0.98:0.72:0.41:0.52), (DABRAFENIB MESYLATE:96:11032:45:137:0.98:0.68:0.41:0.51), (POLYETHYLENE GLYCOL 3350POTASSIUM CHLORIDE\SODIUM BICARBONATE\SODIUM CHLORIDE\SODIUM SULFATE ANHYDROUS:94:11034:43:139:0.98:0.69:0.40:0.51), (ICOSAPENT:70:11058:12:170:0.98: 0.85:0.29:0.43), (EDARAVONE:62:11066:30:152:0.98:0.67:0.29:0.41), (BROMOPRIDE:42: 11086:3:179:0.98:0.93:0.19:0.32), (SODIUM LACTATE:24:11104:0:182:0.98:1.00:0.12:0.21), (RILMENIDINE PHOSPHATE:106:11021:35:148:0.98:0.75:0.42:0.54), (BETAMETHASONECALCIPOTRIE NE:90:11037:109:74:0.98:0.45:0.55:0.50), (METHENOLONE ENANTHATE:83:11044:126:57:0.98:0.40:0.59:0.48), (DANTROLENE SODIUM:72:11055:82:101:0.98:0.47:0.42:0.44), (CEFMETAZOLE SODIUM:44:11083:3:180:0.98:0.94:0.20:0.32), (CARNITINE:35:11092:4:179:0.98:0.90:0.1 6:0.28), (CALCIUM\MAGNESIUM\ZINC:113:11013:113:71:0.98:0.50:0.61:0.55), (METFO RMIN HYDROCHLORIDESAXAGLIPTIN HYDROCHLORIDE:107:11019:30:154:0.98:0.78:0.41:0.54), (OXACILLIN SODIUM:34:11092:2:182:0.98:0.94:0.16:0.27), (CLAVULANIC ACID:23:11103:0:184:0.98:1.00:0.11:0.20), (SARPOGRELATE:138:10987:36:149:0.98:0.79: 0.48:0.60), (TAMOXIFEN CITRATE:126:10999:48:137:0.98:0.72:0.48:0.58), (ABACAVIR:109:11016:10:175:0.98:0.9 2:0.38:0.54), (CARBOMER:107:11018:23:162:0.98:0.82:0.40:0.54), (PERINDOPRIL ARGININE:38:11087:7:178:0.98:0.84:0.18:0.29), (PHENIRAMINE:9:11116:0:185:0.98:1.00: 0.05:0.09), (HERBALSSAW PALMETTO:143:10981:35:151:0.98:0.80:0.49:0.61), (HOMATROPINE METHYLBROMIDEHYDROCODONE BITARTRATE:37:11087:7:179:0.98:0.84:0.17:0.28), (CHLORPROMAZINE HYDROCHLORIDE:29:11095:6:180:0.98:0.83:0.14:0.24), (AMILORIDE HYDROCHLORIDE:18:11106:2:184:0.98:0.90:0.09:0.16), (ERYTHROMYCIN ETHYLSUCCINATE:17:11107:1:185:0.98:0.94:0.08:0.15), (BENDAMUSTINE HYDROCHLORIDE:353:10770:34:153:0.98:0.91:0.70:0.79), (BLINATUMOMAB:129:1099 4:102:85:0.98:0.56:0.60:0.58), (MITIGLINIDE:90:11033:7:180:0.98:0.93:0.33:0.49), (IRSO GLADINE MALEATE:86:11037:5:182:0.98:0.95:0.32:0.48), (ASENAPINE MALEATE:74:11049:5:182:0.98:0.94:0.29:0.44), (DISTIGMINE BROMIDE:69:11054:11:176:0.98:0.86:0.28:0.42), (THROMBOMODULIN ALFA:58:11065:15:172:0.98:0.79:0.25:0.38), (ARGATROBAN:55:11068:5:182:0.98:0.92:0. 23:0.37), (DARIFENACIN HYDROBROMIDE:46:11077:3:184:0.98:0.94:0.20:0.33), (SODIUM:46:11077:3:184:0.98:0. 94:0.20:0.33), (SULFADIAZINE:28:11095:2:185:0.98:0.93:0.13:0.23), (DOXYLAMINE SUCCINATE:138:10984:43:145:0.98:0.76:0.49:0.59), (ESTRIOL:104:11018:25:163:0.98:0.8 1:0.39:0.53), (INDOMETHACININDOMETHACIN SODIUM:27:11095:0:188:0.98:1.00:0.13:0.22), (DELORAZEPAM:14:11108:0:188:0.98:1.00: 0.07:0.13), (NARATRIPTAN HYDROCHLORIDE:100:11021:31:158:0.98:0.76:0.39:0.51), (CALCIUM CHLORIDE ANHYDROUSPOTASSIUM CHLORIDE\SODIUM CHLORIDE\SODIUM LACTATE:52:11069:8:181:0.98:0.87:0.22:0.35), (AVELUMAB:30:11091:3:186:0.98:0.91:0 0.14:0.24), (SODIUM MONOFLUOROPHOSPHATE:20:11101:1:188:0.98:0.95:0.10:0.17), (OXYMETAZOLINE HYDROCHLORIDE:17:11104:0:189:0.98:1.00:0.08:0.15), (ALISKIREN HEMIFUMARATE:175:10945:89:101:0.98:0.66:0.63:0.65), (ANIMALIS LACTIS:52:11068:7:183:0.98:0.88:0.22:0.35), (AGOMELATINE:43:11077:2:188:0.98:0.96: 0.19:0.31), (LINAGLIPTINMETFORMIN HYDROCHLORIDE:33:11087:1:189:0.98:0.97:0.15:0.26), (PHENOL:16:11104:0:190:0.98:1 0.00:0.08:0.14), (DIHYDROCODEINE BITARTRATE:14:11106:1:189:0.98:0.93:0.07:0.13), (CEFOXITIN SODIUM:8:11112:0:190:0.98:1.00:0.04:0.08), (DOCUSATE SODIUMSENNOSIDESSENNOSIDES A AND B:29:11090:0:191:0.98:1.00:0.13:0.23), (LANREOTIDE ACETATE:194:10924:47:145:0.98:0.80:0.57:0.67), (CALCIUM\MAGNESIUM:153:10965:9 3:99:0.98:0.62:0.61:0.61), (ENALAPRIL MALEATEHYDROCHLOROTHIAZIDE:113:11005:28:164:0.98:0.80:0.41:0.54), (CEFCAP ENE PIVOXIL HYDROCHLORIDE:69:11049:5:187:0.98:0.93:0.27:0.42), (IMIDAPRIL:113:11004:9:184:0. 98:0.93:0.38:0.54), (FORMOTEROL FUMARATE DIHYDRATE:95:11022:9:184:0.98:0.91:0.34:0.50), (CISATRACURIUM BESYLATE:48:11069:2:191:0.98:0.96:0.20:0.33), (AZELAIC ACID:45:11072:0:193:0.98:1.00:0.19:0.32), (ALBENDAZOLE:26:11090:3:191:0.98:0.90:0. 12:0.21), (PENTAZOCINE HYDROCHLORIDE:20:11096:2:192:0.98:0.91:0.09:0.17), (DARUNAVIR:134:10981:3:192: 0.98:0.98:0.41:0.58), (ACETAMINOPHENDIPHENHYDRAMINE HYDROCHLORIDE:115:11000:14:181:0.98:0.89:0.39:0.54), (COLISTIMETHATE SODIUM:99:11016:57:138:0.98:0.63:0.42:0.50), (ASPIRINBUTALBITALCAFFEINE:95:1 1020:10:185:0.98:0.90:0.34:0.49), (KETAMINE HYDROCHLORIDE:94:11021:14:181:0.98:0.87:0.34:0.49), (LENVATINIB:90:11025:60:135: 0.98:0.60:0.40:0.48), (CALCIUM ASPARTATE:69:11046:3:192:0.98:0.96:0.26:0.41), (PREDNISONE ACETATE:38:11077:4:191:0.98:0.90:0.17:0.28), (OCRELIZUMAB:30:11085:5:190:0.98:0.8 6:0.14:0.24), (RITONAVIR:618:10496:0:196:0.98:1.00:0.76:0.86), (ACETAMINOPHENC AFFEINEOPIUM:114:11000:39:157:0.98:0.75:0.42:0.54), (BUTYLSCOPOLAMINE:57:110 57:7:189:0.98:0.89:0.23:0.37), (LORCASERIN HYDROCHLORIDE:53:11061:3:193:0.98:0.95:0.22:0.35), (ORLISTAT:44:11070:4:192:0.98: 0.92:0.19:0.31), (MAGNESIUM CARBONATE:37:11077:2:194:0.98:0.95:0.16:0.27), (GUANFACINE:170:10943:85:112:0.98: 0.67:0.60:0.63), (CABOZANTINIB S-MALATE:70:11043:60:137:0.98:0.54:0.34:0.42), (MOSAPRIDE:30:11083:0:197:0.98:1.00: 0.13:0.23), (PNEUMOCOCCAL 7-VALENT CONJUGATE VACCINE (DIPHTHERIA CRM197 PROTEIN):29:11084:0:197:0.98:1.00:0.13:0.23), (ORPHENADRINE CITRATE:22:11091:0:197:0.98:1.00:0.10:0.18), (AMINOPHYLLINE:92:11020:14:184:0.98: 0.87:0.33:0.48), (BLACK COHOSH:66:11046:4:194:0.98:0.94:0.25:0.40), (SPIRAMYCIN:65:11047:13:185:0.98:0.83: 0.26:0.40), (CEFOTAXIMECEFOTAXIME SODIUM:58:11054:77:121:0.98:0.43:0.32:0.37), (FOSFOMYCIN SODIUM:28:11084:5:193:0.98:0.85:0.13:0.22), (CALCIUM CARBONATEFAMOTIDINEMAGNESIUM HYDROXIDE:12:11100:0:198:0.98:1.00:0.06:0.11), (ZAFIRLUKAST:182:10929:57:142:0.9 8:0.76:0.56:0.65), (RIFABUTIN:72:11039:13:186:0.98:0.85:0.28:0.42), (PHENOBARBITAL SODIUM:67:11044:21:178:0.98:0.76:0.27:0.40), (BREXPIPRAZOLE:61:11050:2:197:0.98:0 0.97:0.24:0.38), (FLUOCINOLONE ACETONIDE:146:10964:37:163:0.98:0.80:0.47:0.59), (EDOXABAN TOSILATE MONOHYDRATE:101:11009:25:175:0.98:0.80:0.37:0.50), (DEXCHLORPHENIRAMINE:2 0:11090:0:200:0.98:1.00:0.09:0.17), (LOXAPINE SUCCINATE:14:11096:0:200:0.98:1.00:0.07:0.12), (CHROMIC CHLORIDECHROMIUM:57:11052:3:198:0.98:0.95:0.22:0.36), (GEMTUZUMAB OZOGAMICIN:44:11065:3:198:0.98:0.94:0.18:0.30), (IMIQUIMOD:141:10967:22:180:0.98: 0.87:0.44:0.58), (OLOPATADINE:136:10972:46:156:0.98:0.75:0.47:0.57), (NEPAFENAC:1 30:10978:9:193:0.98:0.94:0.40:0.56), (ANTIHEMOPHILIC FACTOR, HUMAN RECOMBINANT:57:11051:3:199:0.98:0.95:0.22:0.36), (OSELTAMIVIR:223:10884:83:120: 0.98:0.73:0.65:0.69), (SUFENTANIL:103:11004:16:187:0.98:0.87:0.36:0.50), (LEVOMEF OLATE CALCIUM:172:10934:36:168:0.98:0.83:0.51:0.63), (VARDENAFIL HYDROCHLORIDE TRIHYDRATE:127:10979:25:179:0.98:0.84:0.42:0.55), (CEFPODOXIME PROXETIL:125:10981:38:166:0.98:0.77:0.43:0.55), (TIPIRACIL HYDROCHLORIDETRIFLURIDINE:117:10989:39:165:0.98:0.75:0.41:0.53), (ELOTUZUM AB:116:10990:128:76:0.98:0.48:0.60:0.53), (GABAPENTIN ENACARBIL:39:11067:3:201:0.98:0.93:0.16:0.28), (DESOXIMETASONE:110:10995:8:197: 0.98:0.93:0.36:0.52), (GARENOXACIN MESYLATE:56:11049:1:204:0.98:0.98:0.22:0.35), (BOSUTINIB MONOHYDRATE:39:11066:5:200:0.98:0.89:0.16:0.28), (FLURAZEPAMFLURAZEPAM HYDROCHLORIDE:122:10982:50:156:0.98:0.71:0.44:0.54), (MAGNESIUM GLUCONATE:116:10988:19:187:0.98:0.86:0.38:0.53), (ANTITHROMBIN III HUMAN:37:11067:0:206:0.98:1.00:0.15:0.26), (PITAVASTATIN:12:11092:0:206:0.98:1.00: 0.06:0.10), (NALOXONE HYDROCHLORIDETILIDINE HYDROCHLORIDE:173:10930:68:139:0.98:0.72:0.55:0.63), (BECLOMETHASONEFORM OTEROL:143:10960:32:175:0.98:0.82:0.45:0.58), (NIRAPARIB:137:10966:112:95:0.98:0.55: 0.59:0.57), (TESTOSTERONE UNDECANOATE:92:11011:96:111:0.98:0.49:0.45:0.47), (PHENTERMINE HYDROCHLORIDETOPIRAMATE:56:11047:5:202:0.98:0.92:0.22:0.35), (SODIUM BICARBONATE\SODIUM PHOSPHATE, MONOBASIC:39:11064:2:205:0.98:0.95:0.16:0.27), (CANRENONE:20:11083:0:207:0.98:1. 00:0.09:0.16), (NADROPARIN:15:11088:0:207:0.98:1.00:0.07:0.13), (CALCIUM CARBONATEVITAMIN D:415:10687:74:134:0.98:0.85:0.76:0.80), (TENOFOVIR DISOPROXIL FUMARATE:290:10812:0:208:0.98:1.00:0.58:0.74), (CALCIPOTRIENE:193:10909:42:166:0 0.98:0.82:0.54:0.65), (BETHANECHOL CHLORIDE:99:11003:52:156:0.98:0.66:0.39:0.49), (ELTROMBOPAG OLAMINE:93:11009:64:144:0.98:0.59:0.39:0.47), (EFINACONAZOLE:87:11015:19:189:0.9 8:0.82:0.32:0.46), (DEXMETHYLPHENIDATE HYDROCHLORIDE:66:11036:12:196:0.98:0.85:0.25:0.39), (COCAINE:19:11083:0:208:0.98: 1.00:0.08:0.15), (GUANFACINE HYDROCHLORIDE:178:10923:69:140:0.98:0.72:0.56:0.63), (IDARUBICIN:126:10975:13:1 96:0.98:0.91:0.39:0.55), (PYRIDOSTIGMINE:46:11055:11:198:0.98:0.81:0.19:0.31), (IOPR OMIDE:28:11073:5:204:0.98:0.85:0.12:0.21), (LEVALBUTEROL:176:10924:35:175:0.98:0. 83:0.50:0.63), (DOBUTAMINE HYDROCHLORIDE:119:10981:48:162:0.98:0.71:0.42:0.53), (FOSFOMYCIN:108:10992:61: 149:0.98:0.64:0.42:0.51), (COLESTIPOL HYDROCHLORIDE:56:11044:0:210:0.98:1.00:0.21:0.35), (PIRENOXINE:45:11055:1:209:0. 98:0.98:0.18:0.30), (OSPEMIFENE:156:10943:41:170:0.98:0.79:0.48:0.60), (CEVIMELINE HYDROCHLORIDE:125:10974:33:178:0.98:0.79:0.41:0.54), (PATIROMER:123:10976:137: 74:0.98:0.47:0.62:0.54), (RALTEGRAVIR:121:10978:6:205:0.98:0.95:0.37:0.53), (METHO XY POLYETHYLENE GLYCOL-EPOETIN BETA:101:10998:19:192:0.98:0.84:0.34:0.49), (MINERALS:59:11040:4:207:0.98:0.94:0.22: 0.36), (CIPROFLOXACIN HYDROCHLORIDEDEXAMETHASONE:43:11056:0:211:0.98:1.00:0.17:0.29), (ISRADIPI NE:24:11075:1:210:0.98:0.96:0.10:0.19), (PROCATEROL HYDROCHLORIDE:69:11029:10:202:0.98:0.87:0.25:0.39), (DOLASETRON MESYLATE:65:11033:132:80:0.98:0.33:0.45:0.38), (TACROLIMUSTACROLIMUS ANHYDROUS:1040:10057:0:213:0.98:1.00:0.83:0.91), (ACETAMINOPHENPROPOXYPH ENE HYDROCHLORIDE:200:10897:71:142:0.98:0.74:0.58:0.65), (REMIFENTANIL:148:10949:1 0:203:0.98:0.94:0.42:0.58), (FERROUS SULFATE ANHYDROUS:125:10972:23:190:0.98:0.84:0.40:0.54), (SEVELAMER:81:11016:15:198:0.9 8:0.84:0.29:0.43), (RILMENIDINE:62:11035:5:208:0.98:0.93:0.23:0.37), (PIRACETAM:61: 11036:5:208:0.98:0.92:0.23:0.36), (BIPERIDEN:56:11041:8:205:0.98:0.88:0.21:0.34), (AM MONIUM GLYCYRRHIZATE:51:11046:3:210:0.98:0.94:0.20:0.32), (DOBUTAMINEDOBUTAMINE HYDROCHLORIDE:159:10937:7:207:0.98:0.96:0.43:0.60), (AMINO ACIDSDEXTROSEELECTROLYTES NOS:116:10980:105:109:0.98:0.52:0.52:0.52), (VECURONIUM BROMIDE:102:10994:24:190:0.98:0.81:0.35:0.49), (BETAXOLOL HYDROCHLORIDE:96:11000:15:199:0.98:0.86:0.33:0.47), (CHLORDIAZEPOXIDE:83:110 13:9:205:0.98:0.90:0.29:0.44), (DOCUSATE SODIUMSENNOSIDES A AND B:66:11030:5:209:0.98:0.93:0.24:0.38), (PROPYLENE GLYCOL:61:11035:1:213:0.98:0.98:0.22:0.36), (DIFLUCORTOLONE VALERATE:46:11050:1:213:0.98:0.98:0.18:0.30), (RESVERATROL:29:11067:1:213:0.98:0 0.97:0.12:0.21), (MEBEVERINE:21:11075:1:213:0.98:0.95:0.09:0.16), (RUXOLITINIB:610: 10485:1:214:0.98:1.00:0.74:0.85), (FENOTEROL HYDROBROMIDE:152:10943:48:167:0.98:0.76:0.48:0.59), (DEXTROAMPHETAMINE SULFATE:140:10955:71:144:0.98:0.66:0.49:0.57), (ASPIRINMAGNESIUM OXIDE:112:10983:49:166:0.98:0.70:0.40:0.51), (HYALURONIC ACID:77:11018:8:207:0.98:0.91:0.27:0.42), (TRIMETAZIDINE:50:11045:6:209:0.98:0.89:0 0.19:0.32), (TREMELIMUMAB:5:11090:0:215:0.98:1.00:0.02:0.04), (INTERFERON ALFA-2A:60:11034:154:62:0.98:0.28:0.49:0.36), (ALEMTUZUMAB:398:10695:68:149:0.98:0.85: 0.73:0.79), (CARBIDOPA:139:10954:39:178:0.98:0.78:0.44:0.56), (PEGASPARGASE:126:1 0967:14:203:0.98:0.90:0.38:0.54), (DEXMEDETOMIDINE:39:11054:1:216:0.98:0.98:0.15: 0.26), (LEVOLEUCOVORIN CALCIUM:187:10905:113:105:0.98:0.62:0.64:0.63), (PYRIDOXAL:98:10994:14:204:0.98:0. 88:0.32:0.47), (ROMIDEPSIN:11:11081:0:218:0.98:1.00:0.05:0.09), (PASIREOTIDE:100:10 991:40:179:0.98:0.71:0.36:0.48), (MEPOLIZUMAB:94:10997:172:47:0.98:0.35:0.67:0.46), (PANITUMUMAB:226:10864:56:164:0.98:0.80:0.58:0.67), (FONDAPARINUX:166:10924:4 1:179:0.98:0.80:0.48:0.60), (UMECLIDINIUM BROMIDE:155:10935:101:119:0.98:0.61:0.57:0.58), (PENTAMIDINE ISETHIONATE:148:10942:30:190:0.98:0.83:0.44:0.57), (AZELNIDIPINE:141:10949:19:201: 0.98:0.88:0.41:0.56), (CERITINIB:127:10963:44:176:0.98:0.74:0.42:0.54), (HYDROCHLO ROTHIAZIDERAMIPRIL:87:11003:18:202:0.98:0.83:0.30:0.44), (FIDAXOMICIN:16:1107 4:0:220:0.98:1.00:0.07:0.13), (DOXOFYLLINE:12:11078:0:220:0.98:1.00:0.05:0.10), (PAZ OPANIB:262:10827:2:219:0.98:0.99:0.54:0.70), (CABERGOLINE:238:10851:0:221:0.98:1.0 0:0.52:0.68), (FENOTEROL HYDROBROMIDEIPRATROPIUM BROMIDE:165:10924:60:161:0.98:0.73:0.51:0.60), (CHLORDIAZEPOXIDE HYDROCHLORIDE:63:11026:0:221:0.98:1.00:0.22:0.36), (ECHINACEA, UNSPECIFIED:48:11041:1:220:0.98:0.98:0.18:0.30), (PROPACETAMOL HYDROCHLORIDE:12:11077:0:221:0.98:1.00:0.05:0.10), (ESOMEPRAZOLE MAGNESIUMNAPROXEN:217:10871:61:161:0.98:0.78:0.57:0.66), (LACTASE:186:10902: 77:145:0.98:0.71:0.56:0.63), (PRISTINAMYCIN:88:11000:48:174:0.98:0.65:0.34:0.44), (B ECLOMETHASONE DIPROPIONATE MONOHYDRATE:24:11064:1:221:0.98:0.96:0.10:0.18), (VELIPARIB:12:11076:0:222:0.98: 1.00:0.05:0.10), (ETHAMSYLATE:5:11083:0:222:0.98:1.00:0.02:0.04), (MORPHINE SULFATE\NALTREXONE HYDROCHLORIDE:141:10946:59:164:0.98:0.71:0.46:0.56), (CEFADROXIL:76:11011:2:22 1:0.98:0.97:0.26:0.41), (AMINO ACIDSCARBOHYDRATESELECTROLYTES NOSVITAMINS:60:11027:9:214:0.98:0.87:0.22:0.35), (TRIMEPRAZINE:49:11038:3:220:0. 98:0.94:0.18:0.31), (ABACAVIR SULFATELAMIVUDINE:232:10854:5:219:0.98:0.98:0.51:0.67), (CYPROHEPTADINE HYDROCHLORIDE:124:10962:33:191:0.98:0.79:0.39:0.53), (IVERMECTIN:108:10978:8:2 16:0.98:0.93:0.33:0.49), (INTERFERON:105:10981:110:114:0.98:0.49:0.48:0.48), (CEFDIT OREN PIVOXIL:99:10987:9:215:0.98:0.92:0.32:0.47), (DACLATASVIR:132:10953:22:203:0.98:0. 86:0.39:0.54), (METOPIMAZINE:31:11054:2:223:0.98:0.94:0.12:0.22), (DEXTROSESODI UM CHLORIDE\SODIUM LACTATE:27:11058:0:225:0.98:1.00:0.11:0.19), (CALCIUM CARBONATECHOLECALCIFEROLMAGNESIUM CARBONATE:152:10932:18:208:0.98:0.89:0.42:0.57), (ANTIHISTAMINES NOS:113:10971:95:131:0.98:0.54:0.46:0.50), (CARPERITIDE:75:11009:7:219:0.98:0.91:0.2 6:0.40), (BENZOCAINE:65:11019:20:206:0.98:0.76:0.24:0.37), (CALCITONIN:171:10912:9 0:137:0.98:0.66:0.56:0.60), (GINGER:104:10979:11:216:0.98:0.90:0.33:0.48), (VINORELB INEVINORELBINE TARTRATE:101:10982:39:188:0.98:0.72:0.35:0.47), (DIPHENHYDRAMINE HYDROCHLORIDELIDOCAINE HYDROCHLORIDENYSTATIN:90:10993:13:214:0.98:0.87:0.30:0.44), (GLYCERINPAR AFFIN:68:11015:2:225:0.98:0.97:0.23:0.37), (DESIPRAMINE:38:11045:1:226:0.98:0.97:0.1 4:0.25), (SOYBEAN OIL:16:11067:1:226:0.98:0.94:0.07:0.12), (SALMETEROL:190:10892:52:176:0.98:0.79:0.5 2:0.63), (LAPATINIB DITOSYLATE:135:10947:111:117:0.98:0.55:0.54:0.54), (GEFITINIB:130:10952:34:194:0.98: 0.79:0.40:0.53), (BUPIVACAINE HYDROCHLORIDE:105:10977:32:196:0.98:0.77:0.35:0.48), (DIMETHINDENE:84:10998:7: 221:0.98:0.92:0.28:0.42), (EPHEDRINE:65:11017:8:220:0.98:0.89:0.23:0.36), (TIAPRIDE HYDROCHLORIDE:30:11052:1:227:0.98:0.97:0.12:0.21), (CASPOFUNGIN:242:10839:47:1 82:0.98:0.84:0.57:0.68), (DIGITOXIN:172:10909:103:126:0.98:0.63:0.58:0.60), (SODIUM PHOSPHATE, DIBASIC\SODIUM PHOSPHATE, MONOBASIC:172:10909:73:156:0.98:0.70:0.52:0.60), (BECLOMETHASONE:129:10952:40: 189:0.98:0.76:0.41:0.53), (MIGLITOL:128:10953:10:219:0.98:0.93:0.37:0.53), (ALOE VERA LEAF:44:11037:0:229:0.98:1.00:0.16:0.28), (HOMEOPATHICS:17:11064:0:229:0.98:1.00:0 0.07:0.13), (DABRAFENIB:144:10936:71:159:0.98:0.67:0.48:0.56), (ACITRETIN:132:10948: 40:190:0.98:0.77:0.41:0.53), (STARCH, WHEAT:126:10954:19:211:0.98:0.87:0.37:0.52), (PRANLUKAST:121:10959:28:202:0.98:0. 81:0.37:0.51), (CLOMIPRAMINE:74:11006:9:221:0.98:0.89:0.25:0.39), (GUAIFENESINP SEUDOEPHEDRINE HYDROCHLORIDE:36:11044:0:230:0.98:1.00:0.14:0.24), (RIZATRIPTAN:180:10899:58:17 3:0.98:0.76:0.51:0.61), (TETRABENAZINE:82:10997:15:216:0.98:0.85:0.28:0.42), (DOCU SATE SODIUM\SENNA LEAF:69:11010:1:230:0.98:0.99:0.23:0.37), (ACYCLOVIR SODIUM:56:11023:1:230:0.98:0.98:0.20:0.33), (DULAGLUTIDE:272:10806:38:194:0.98:0. 88:0.58:0.70), (POTASSIUM PHOSPHATE:51:11027:1:231:0.98:0.98:0.18:0.31), (PIMECROLIMUS:160:10917:62:171:0. 98:0.72:0.48:0.58), (TRAMETINIB DIMETHYL SULFOXIDE:87:10990:40:193:0.98:0.69:0.31:0.43), (TELAPREVIR:40:11037:4:229:0.98:0. 91:0.15:0.26), (CEFTRIAXONE:1304:9772:0:234:0.98:1.00:0.85:0.92), (DEXAMETHASO NE ACETATE:195:10881:102:132:0.98:0.66:0.60:0.63), (EPIRUBICIN HYDROCHLORIDE:69:11007:20:214:0.98:0.78:0.24:0.37), (OMALIZUMAB:508:10567:0:2 35:0.98:1.00:0.68:0.81), (LAPINE T-LYMPHOCYTE IMMUNE GLOBULIN:377:10698:5:230:0.98:0.99:0.62:0.76), (IMMUNE GLOBULIN NOS:130:10945:51:184:0.98:0.72:0.41:0.53), (DIOSMIN:88:10987:11:224:0.98:0.89:0.28:0. 43), (DROPERIDOL:72:11003:7:228:0.98:0.91:0.24:0.38), (RILMAZAFONE HYDROCHLORIDE:44:11031:0:235:0.98:1.00:0.16:0.27), (ALUMINUM HYDROXIDE:33:11042:2:233:0.98:0.94:0.12:0.22), (TRANDOLAPRIL:161:10913:41:195:0. 98:0.80:0.45:0.58), (SULINDAC:147:10927:28:208:0.98:0.84:0.41:0.55), (FISH OILTOCOPHEROL:71:11003:5:231:0.98:0.93:0.24:0.38), (ASCORBIC ACIDCALCIUM PANTOTHENATE:47:11027:3:233:0.98:0.94:0.17:0.28), (FLUMAZENIL:17:11057:2:234:0. 98:0.89:0.07:0.13), (TROPISETRON HYDROCHLORIDE:6:11068:0:236:0.98:1.00:0.02:0.05), (IXAZOMIB:192:10881:81:156:0.9 8:0.70:0.55:0.62), (DASABUVIROMBITASVIRPARITAPREVIRRITONAVIR:170:10903: 36:201:0.98:0.83:0.46:0.59), (PNEUMOCOCCAL VACCINE, POLYVALENT 23:139:10934:24:213:0.98:0.85:0.39:0.54), (NIACINAMIDE:69:11004:5:232:0.98:0.93:0.23: 0.37), (FELBINAC:49:11024:3:234:0.98:0.94:0.17:0.29), (BUPROPION HYDROCHLORIDE\NALTREXONE HYDROCHLORIDE:40:11033:0:237:0.98:1.00:0.14:0.25), (CHLORAMBUCIL:173:10899:5 7:181:0.98:0.75:0.49:0.59), (CASANTHRANOLDOCUSATE SODIUM:148:10924:41:197:0.98:0.78:0.43:0.55), (ACTIVATED CHARCOAL:134:10938:53:185:0.98:0.72:0.42:0.53), (MINODRONIC ACID:105:10967:10:228:0.98:0.91:0.32:0.47), (AGALSIDASE BETA:102:10970:179:59:0.98:0.36:0.63:0.46), (CYANOCOBALAMINPYRIDOXINETHIA MINE:38:11034:1:237:0.98:0.97:0.14:0.24), (ETOPOSIDE:1062:10009:2:237:0.98:1.00:0.82: 0.90), (HYDROCORTISONE ACETATE:205:10866:67:172:0.98:0.75:0.54:0.63), (GLYCOPYRRONIUM:178:10893:59:18 0:0.98:0.75:0.50:0.60), (PREDNISOLONE SODIUM PHOSPHATE:129:10942:108:131:0.98:0.54:0.50:0.52), (BROMFENAC SODIUM:114:10957:6:233:0.98:0.95:0.33:0.49), (AMPHETAMINE:78:10993:1:238:0.98:0.9 9:0.25:0.39), (NAFTOPIDIL:76:10995:2:237:0.98:0.97:0.24:0.39), (SUCCINYLCHOLINE:7 3:10998:1:238:0.98:0.99:0.23:0.38), (VARICELLA-ZOSTER VIRUS STRAIN OKA/MERCK LIVE ANTIGEN:71:11000:1:238:0.98:0.99:0.23:0.37), (EPERISONE HYDROCHLORIDE:97:10973:13:227:0.98:0.88:0.30:0.45), (TEMSIROLIMUS:83:10987:35: 205:0.98:0.70:0.29:0.41), (CANRENOATE POTASSIUM:69:11001:4:236:0.98:0.95:0.23:0.37), (PIMAVANSERIN TARTRATE:50:11020:4:236:0.98:0.93:0.17:0.29), (IBANDRONIC ACID:44:11026:0:240:0.98:1.00:0.15:0.27), (AMLODIPINE BESYLATEATORVASTATIN CALCIUM:190:10879:38:203:0.98:0.83:0.48:0.61), (RAMUCIRUMAB:135:10934:77:164:0. 98:0.64:0.45:0.53), (BUDESONIDEFORMOTEROL:121:10948:23:218:0.98:0.84:0.36:0.50), (LECITHIN:86:10983:9:232:0.98:0.91:0.27:0.42), (CHROMIUM PICOLINATE:48:11021:1:240:0.98:0.98:0.17:0.28), (RETINOL:166:10902:75:167:0.98:0.69: 0.50:0.58), (PIRFENIDONE:161:10907:35:207:0.98:0.82:0.44:0.57), (VINORELBINE TARTRATE:151:10917:76:166:0.98:0.67:0.48:0.56), (FORMOTEROL FUMARATE\MOMETASONE FUROATE:96:10972:206:36:0.98:0.32:0.73:0.44), (FERRIC CARBOXYMALTOSE:71:10997:10:232:0.98:0.88:0.23:0.37), (ISAVUCONAZONIUM SULFATE:32:11036:5:237:0.98:0.86:0.12:0.21), (BETHANECHOL:30:11038:1:241:0.98:0.9 7:0.11:0.20), (COLISTIN:96:10971:36:207:0.98:0.73:0.32:0.44), (OMEPRAZOLE\SODIUM BICARBONATE:94:10973:4:239:0.98:0.96:0.28:0.44), (CALCIUM CARBONATEMAGNESIUM CARBONATEMAGNESIUM TRISILICATE:54:11013:4:239:0.98:0.93:0.18:0.31), (CETYLPYRIDINIUM CHLORIDE OR SODIUM FLUORIDE OR SODIUM MONOFLUOROPHOSPHATE OR GLUCOSE OXIDASELACTOFERRINLACTOPEROXIDASE BOVINELYSOZYME:39:11028:3:240:0.98:0.93:0.14:0.24), (ESTROGENS:167:10899:49:1 95:0.98:0.77:0.46:0.58), (WARFARIN POTASSIUM:154:10912:32:212:0.98:0.83:0.42:0.56), (ACEBUTOLOL HYDROCHLORIDE:120:10946:26:218:0.98:0.82:0.36:0.50), (DEXTROAMPHETAMINE:10 2:10964:19:225:0.98:0.84:0.31:0.46), (POLYETHYLENE GLYCOL 400PROPYLENE GLYCOL:37:11029:0:244:0.98:1.00:0.13:0.23), (SEVELAMER HYDROCHLORIDE:215:10849:43:203:0.98:0.83:0.51:0.64), (TOLTERODINE:195:10869:3 2:214:0.98:0.86:0.48:0.61), (BETAMETHASONE DIPROPIONATECLOTRIMAZOLE:142:10922:31:215:0.98:0.82:0.40:0.54), (AMLODIPINE\PERINDOPRIL:132:10932:43:203:0.98:0.75:0.39:0.52), (ASCORBIC ACIDASPIRIN:26:11038:0:246:0.98:1.00:0.10:0.17), (ETORICOXIB:361:10702:107:140:0.9 8:0.77:0.72:0.75), (LOTEPREDNOL ETABONATE:264:10799:11:236:0.98:0.96:0.53:0.68), (INTERFERON ALFA-2B:103:10960:97:150:0.98:0.52:0.41:0.45), (ELECTROLYTES NOS:11:11052:1:246:0.98:0.92:0.04:0.08), (VINCRISTINE:1120:9942:1:247:0.98:1.00:0.82: 0.90), (AMLODIPINEVALSARTAN:173:10889:136:112:0.98:0.56:0.61:0.58), (AMLODIPINE BESYLATEHYDROCHLOROTHIAZIDEVALSARTAN:166:10896:68:180:0.98:0.71:0.48: 0.57), (DORZOLAMIDE HYDROCHLORIDE:161:10901:34:214:0.98:0.83:0.43:0.56), (DICLOFENAC SODIUMMISOPROSTOL:148:10914:15:233:0.98:0.91:0.39:0.54), (CHLORZOXAZONE:69: 10993:1:247:0.98:0.99:0.22:0.36), (DORZOLAMIDE:211:10850:29:220:0.98:0.88:0.49:0.6 3), (COLESTIPOL:135:10926:8:241:0.98:0.94:0.36:0.52), (PENTAZOCINE:43:11018:4:245: 0.98:0.91:0.15:0.26), (VENETOCLAX:479:10581:1:249:0.98:1.00:0.66:0.79), (ALFUZOSIN HYDROCHLORIDE:247:10813:26:224:0.98:0.90:0.52:0.66), (TERBUTALINE:166:10894:74: 176:0.98:0.69:0.49:0.57), (DORNASE ALFA:141:10919:3:247:0.98:0.98:0.36:0.53), (CALCIUM LACTATE:108:10952:20:230:0.98:0.84:0.32:0.46), (CARBIDOPAENTACAPONELEVOD OPA:101:10959:8:242:0.98:0.93:0.29:0.45), (CYAMEMAZINE:96:10963:35:216:0.98:0.73: 0.31:0.43), (ACETAMINOPHENDEXTROMETHORPHAN HYDROBROMIDEDOXYLAMINE SUCCINATE:28:11031:0:251:0.98:1.00:0.10:0.18), (ALISKIREN:201:10857:30:222:0.98:0.8 7:0.48:0.61), (MOXONIDINE:201:10857:41:211:0.98:0.83:0.49:0.61), (TRAMETINIB:162: 10896:54:198:0.98:0.75:0.45:0.56), (.ALPHA.1-PROTEINASE INHIBITOR HUMAN:124:10934:193:59:0.98:0.39:0.68:0.50), (CALCIFEDIOL:60:10998:1:251:0.98:0.98: 0.19:0.32), (PIPERACILLIN SODIUM:38:11020:5:247:0.98:0.88:0.13:0.23), (AMINOCAPROIC ACID:21:11037:2:250:0.98:0.91:0.08:0.14), (FORMOTEROL:228:10829:93:160:0.98:0.71:0 0.59:0.64), (CODEINE PHOSPHATEGUAIFENESIN:168:10889:38:215:0.98:0.82:0.44:0.57), (SOTALOL HYDROCHLORIDE:145:10912:26:227:0.98:0.85:0.39:0.53), (DEXTROMETHORPHAN HYDROBROMIDEQUINIDINE SULFATE:81:10976:12:241:0.98:0.87:0.25:0.39), (ETOMIDATE:56:11001:2:251:0.98:0.97 0.18:0.31), (OLODATEROL HYDROCHLORIDETIOTROPIUM BROMIDE MONOHYDRATE:253:10803:118:136:0.98:0.68:0.65:0.67), (AMLODIPINE BESYLATETELMISARTAN:148:10908:20:234:0.98:0.88:0.39:0.54), (ECONAZOLE:145:1 0911:155:99:0.98:0.48:0.59:0.53), (SOLIFENACIN:81:10975:11:243:0.98:0.88:0.25:0.39), (MAGNESIUM ASPARTATE:47:11009:1:253:0.98:0.98:0.16:0.27), (DANAZOL:170:10885:173:82:0.98:0.5 0:0.67:0.57), (PRAZEPAM:98:10957:12:243:0.98:0.89:0.29:0.43), (LOXOPROFEN SODIUM DIHYDRATE:74:10981:7:248:0.98:0.91:0.23:0.37), (PSEUDOEPHEDRINE:63:10992:3:252: 0.98:0.95:0.20:0.33), (ELUXADOLINE:11:11044:0:255:0.98:1.00:0.04:0.08), (CLOMIPRA MINE HYDROCHLORIDE:166:10888:69:187:0.98:0.71:0.47:0.56), (ATROPINEDIPHENOXYLA TE:140:10914:29:227:0.98:0.83:0.38:0.52), (DIHYDROCODEINE:28:11026:1:255:0.98:0.97: 0.10:0.18), (BIPERIDEN HYDROCHLORIDE:125:10928:18:239:0.98:0.87:0.34:0.49), (PILOCARPINE HYDROCHLORIDE:110:10943:15:242:0.98:0.88:0.31:0.46), (CICLOPIROX:79:10974:7:250: 0.98:0.92:0.24:0.38), (MIANSERIN HYDROCHLORIDE:47:11006:0:257:0.98:1.00:0.15:0.27), (CARBIMAZOLE:162:10890:47: 211:0.98:0.78:0.43:0.56), (DIMETHYL SULFONE:43:11009:0:258:0.98:1.00:0.14:0.25), (NATEGLINIDE:230:10821:105:154:0.98: 0.69:0.60:0.64), (TINZAPARIN:182:10869:60:199:0.98:0.75:0.48:0.58), (SODIUM FERROUS CITRATE:139:10912:17:242:0.98:0.89:0.36:0.52), (EMPAGLIFLOZINLINAGLIPTIN:107:1 0944:241:18:0.98:0.31:0.86:0.45), (CURCUMIN:98:10953:8:251:0.98:0.92:0.28:0.43), (PER INDOPRIL ERBUMINE:96:10955:0:259:0.98:1.00:0.27:0.43), (ZALEPLON:93:10958:5:254:0.98:0.95:0 0.27:0.42), (ALPROSTADIL:309:10741:1:259:0.98:1.00:0.54:0.70), (FOSINOPRIL SODIUM:78:10972:7:253:0.98:0.92:0.24:0.38), (VALERIAN:24:11026:0:260:0.98:1.00:0.08: 0.16), (DOXEPIN HYDROCHLORIDE:244:10805:112:149:0.98:0.69:0.62:0.65), (ZIPRASIDONE HYDROCHLORIDEZIPRASIDONE MESYLATE:224:10825:26:235:0.98:0.90:0.49:0.63), (PALIPERIDONE PALMITATE:206:10843:30:231:0.98:0.87:0.47:0.61), (PRAZOSIN HYDROCHLORIDE:198:10851:75:186:0.98:0.73:0.52:0.60), (RASBURICASE:176:10873:9: 252:0.98:0.95:0.41:0.57), (CANAGLIFLOZINMETFORMIN HYDROCHLORIDE:145:10904:212:49:0.98:0.41:0.75:0.53), (RALOXIFENE:62:10987:0:26 1:0.98:1.00:0.19:0.32), (BACITRACIN ZINCNEOMYCIN SULFATEPOLYMYXIN B SULFATE:20:11029:1:260:0.98:0.95:0.07:0.13), (METFORMIN HYDROCHLORIDEVILDAGLIPTIN:211:10837:86:176:0.98:0.71:0.55:0.62), (ESTRADIO LNORETHINDRONE ACETATE:127:10921:26:236:0.98:0.83:0.35:0.49), (DABIGATRAN:239:10808:131:132:0.98: 0.65:0.64:0.65), (FLUVOXAMINE MALEATE:217:10830:61:202:0.98:0.78:0.52:0.62), (GIMERACILOTERACILTEGAFUR:1 36:10911:100:163:0.98:0.58:0.45:0.51), (IOPAMIDOL:119:10928:14:249:0.98:0.89:0.32:0.4 8), (BICARBONATE ION:101:10946:21:242:0.98:0.83:0.29:0.43), (PREDNISOLONE SODIUM SUCCINATE:94:10953:20:243:0.98:0.82:0.28:0.42), (BICALUTAMIDE:378:10668:62:202:0 0.98:0.86:0.65:0.74), (COD LIVER OIL:240:10806:55:209:0.98:0.81:0.53:0.65), (OLAPARIB:166:10880:61:203:0.98:0.73:0.45: 0.56), (ALOGLIPTIN BENZOATE:147:10899:9:255:0.98:0.94:0.37:0.53), (TICLOPIDINE HYDROCHLORIDE:53:10993:1:263:0.98:0.98:0.17:0.29), (ECONAZOLE NITRATE:19:11027:0:264:0.98:1.00:0.07:0.13), (PRASUGREL HYDROCHLORIDE:382:10663:2:263:0.98:0.99:0.59:0.74), (RALTEGRAVIR POTASSIUM:320:10725:19:246:0.98:0.94:0.57:0.71), (IODINE:162:10883:39:226:0.98:0.81: 0.42:0.55), (REMIFENTANIL HYDROCHLORIDE:103:10942:3:262:0.98:0.97:0.28:0.44), (DOLUTEGRAVIR SODIUM:95:10950:5:260:0.98:0.95:0.27:0.42), (CORTISONE ACETATE:92:10953:18:247:0.98:0.84:0.27:0.41), (PROPAFENONE HYDROCHLORIDE:238:10806:63:203:0.98:0.79:0.54:0.64), (CHLORDIAZEPOXIDE HYDROCHLORIDECLIDINIUM BROMIDE:189:10855:57:209:0.98:0.77:0.47:0.59), (LEVODOPA:142:10902:8:258:0.98:0.9: 0.36:0.52), (CALCIUM CITRATEVITAMIN D:139:10905:14:252:0.98:0.91:0.36:0.51), (CEFUROXIME SODIUM:42:11002:6:260:0.98:0.88:0.14:0.24), (VORINOSTAT:26:11018:0:266:0.98:1.00:0 0.09:0.16), (IMIPRAMINE HYDROCHLORIDE:261:10782:182:85:0.98:0.59:0.75:0.66), (ZIPRASIDONE:51:10992:0:26 7:0.98:1.00:0.16:0.28), (MERCAPTOPURINE:442:10600:5:263:0.98:0.99:0.63:0.77), (BET AMETHASONE VALERATEGENTAMICIN SULFATE:57:10985:3:265:0.98:0.95:0.18:0.30), (DEFLAZACORT:229:10812:115:154:0.98: 0.67:0.60:0.63), (PHENYLEPHRINE HYDROCHLORIDE:177:10864:0:269:0.98:1.00:0.40:0.57), (LOMITAPIDE MESYLATE:137:10904:172:97:0.98:0.44:0.59:0.50), (LABETALOL HYDROCHLORIDE:238:10802:59:211:0.98:0.80:0.53:0.64), (NORETHINDRONE:232:1080 8:60:210:0.98:0.79:0.52:0.63), (TAZOBACTAM:149:10891:39:231:0.98:0.79:0.39:0.52), (R ADIATION THERAPY:147:10893:94:176:0.98:0.61:0.46:0.52), (NALOXONE:131:10909:63:207:0.98:0. 68:0.39:0.49), (CHONDROITIN:63:10977:1:269:0.98:0.98:0.19:0.32), (COCONUT OIL:51:10989:2:268:0.98:0.96:0.16:0.27), (ARGININE:89:10950:8:263:0.98:0.92:0.25:0.40), (TOCOPHERYL NICOTINATE, D-.ALPHA.:75:10964:3:268:0.98:0.96:0.22:0.36), (NITRENDIPINE:71:10968:2:269:0.98:0.9 7:0.21:0.34), (FOSAPREPITANT:38:11001:1:270:0.98:0.97:0.12:0.22), (FINGOLIMOD HYDROCHLORIDE:565:10473:70:202:0.98:0.89:0.74:0.81), (FENOFIBRIC ACID:232:10806:46:226:0.98:0.83:0.51:0.63), (BENZTROPINE MESYLATE:163:10874:31:242:0.98:0.84:0.40:0.54), (LEVOMEFOLATE CALCIUM\METHYLCOBALAMINPYRIDOXAL PHOSPHATE ANHYDROUS:155:10882:16:257:0.98:0.91:0.38:0.53), (FOSCARNET SODIUM:43:10994:1:272:0.98:0.98:0.14:0.24), (DIPHENHYDRAMINE CITRATEIBUPROFEN:17:11020:0:273:0.98:1.00:0.06:0.11), (CETIRIZINE HYDROCHLORIDEPSEUDOEPHEDRINE HYDROCHLORIDE:209:10827:71:203:0.98:0.75:0.51:0.60), (HYDROCHLOROTHIAZIDE LOSARTAN:196:10840:19:255:0.98:0.91:0.43:0.59), (NOREPINEPHRINE BITARTRATE:150:10886:53:221:0.98:0.74:0.40:0.52), (CALCITONIN SALMON:214:10821:120:155:0.98:0.64:0.58:0.61), (QUINAPRILQUINAPRIL HYDROCHLORIDE:178:10857:16:259:0.98:0.92:0.41:0.56), (FOSINOPRIL:118:10917:9:26 6:0.98:0.93:0.31:0.46), (TULOBUTEROL:84:10951:3:272:0.98:0.97:0.24:0.38), (BEPOTAS TINE:82:10953:4:271:0.98:0.95:0.23:0.37), (BERAPROST SODIUM:179:10855:21:255:0.98:0.90:0.41:0.56), (PHLOROGLUCINOL1,3,5-TRIMETHOXYBENZENE:101:10933:20:256:0.98:0.83:0.28:0.42), (FLUNISOLIDE:78:1095 6:1:275:0.98:0.99:0.22:0.36), (DESOGESTREL:42:10992:3:273:0.98:0.93:0.13:0.23), (LEV ALBUTEROL TARTRATE:85:10948:6:271:0.98:0.93:0.24:0.38), (GALANTAMINE:44:10989:1:276:0.98:0 0.98:0.14:0.24), (AMMONIUM LACTATE:180:10852:52:226:0.98:0.78:0.44:0.56), (DEXMEDETOMIDINE HYDROCHLORIDE:150:10882:21:257:0.98:0.88:0.37:0.52), (ACETAMINOPHENOXYCO DONEOXYCODONE HYDROCHLORIDE:248:10783:156:123:0.98:0.61:0.67:0.64), (CEFACLOR:43:10988:0:279: 0.98:1.00:0.13:0.24), (SALMETEROL XINAFOATE:212:10818:55:225:0.98:0.79:0.49:0.60), (QUINAPRIL:209:10821:44:236:0.98: 0.83:0.47:0.60), (LAFUTIDINE:142:10888:13:267:0.98:0.92:0.35:0.50), (SALICYLIC ACID:41:10989:0:280:0.98:1.00:0.13:0.23), (VILAZODONE HYDROCHLORIDE:341:10688:75:206:0.98:0.82:0.62:0.71), (ASPIRINDIHYDROXYALU MINUM AMINOACETATE ANHYDROUSMAGNESIUM CARBONATE:113:10916:30:251:0.98:0.79:0.31:0.45), (BIFIDOBACTERIUM LONGUM SUBSP. INFANTIS:231:10797:32:250:0.98:0.88:0.48:0.62), (IRINOTECAN HYDROCHLORIDE:220:10808:110:172:0.98:0.67:0.56:0.61), (CEFCAPENE PIVOXIL HYDROCHLORIDE HYDRATE:88:10940:3:279:0.98:0.97:0.24:0.38), (PILOCARPINE:172:10855:42:241:0.97:0. 80:0.42:0.55), (DIMETHICONESILICON DIOXIDESIMETHICONE:122:10905:22:261:0.97:0.85:0.32:0.46), (FLUVOXAMINE:78:10 949:7:276:0.97:0.92:0.22:0.36), (MIDODRINE HYDROCHLORIDE:178:10848:62:222:0.97:0.74:0.45:0.56), (BENZBROMARONE:88:1093 8:7:277:0.97:0.93:0.24:0.38), (DAPAGLIFLOZIN PROPANEDIOL:282:10743:143:142:0.97:0.66:0.67:0.66), (LACTOBACILLUS RHAMNOSUS:229:10796:53:232:0.97:0.81:0.50:0.62), (CYPROHEPTADINE:207:10818:72: 213:0.97:0.74:0.49:0.59), (NALOXONE HYDROCHLORIDE:70:10955:6:279:0.97:0.92:0.20:0.33), (POLAPRE\ZINC:91:10932:3:284: 0.97:0.97:0.24:0.39), (DORIPENEM MONOHYDRATE:56:10967:1:286:0.97:0.98:0.16:0.28), (ADENOSINE:75:10947:8:280:0.97: 0.90:0.21:0.34), (BISMUTH SUBSALICYLATE:47:10975:1:287:0.97:0.98:0.14:0.25), (LIRAGLUTIDE:643:10378:1:288: 0.97:1.00:0.69:0.82), (BIFIDOBACTERIUM ANIMALIS SUBSP. LACTIS:95:10926:5:284:0.97:0.95:0.25:0.40), (CARBOXYMETHYLCELLULOSE SODIUMGLYCERIN:51:10970:0:289:0.97:1.00:0.15:0.26), (INDACATEROL:220:10800:51: 239:0.97:0.81:0.48:0.60), (ZOLMITRIPTAN:168:10852:21:269:0.97:0.89:0.38:0.54), (DAB IGATRAN ETEXILATE MESYLATE:1057:9962:0:291:0.97:1.00:0.78:0.88), (DESMOPRESSIN ACETATE:183:10836:2:289:0.97:0.99:0.39:0.56), (RIVASTIGMINE TARTRATE:279:10739:63:229:0.97:0.82:0.55:0.66), (FISH OIL\OMEGA-3 FATTY ACIDS:269:10749:50:242:0.97:0.84:0.53:0.65), (AMILORIDE HYDROCHLORIDEHYDROCHLOROTHIAZIDE:201:10817:48:244:0.97:0.81:0.45:0.58), (IMIPRAMINE:182:10836:13:279:0.97:0.93:0.39:0.55), (MEFENAMIC ACID:179:10839:23:269:0.97:0.89:0.40:0.55), (BETAMETHASONE SODIUM PHOSPHATE:125:10893:16:276:0.97:0.89:0.31:0.46), (VONOPRAZAN FUMURATE:89:10929:6:286:0.97:0.94:0.24:0.38), (ROXITHROMYCIN:65:10953:5:287:0.9 7:0.93:0.18:0.31), (GADOBUTROL:38:10980:8:284:0.97:0.83:0.12:0.21), (RAMOSETRON HYDROCHLORIDE:33:10985:1:291:0.97:0.97:0.10:0.18), (TEMOZOLOMIDE:324:10693:1 1:282:0.97:0.97:0.53:0.69), (VALGANCICLOVIR:299:10718:2:291:0.97:0.99:0.51:0.67), (METHENAMINE HIPPURATE:201:10816:225:68:0.97:0.47:0.75:0.58), (BENZTROPINE:177:10840:19:274:0. 97:0.90:0.39:0.55), (CILNIDIPINE:147:10870:7:286:0.97:0.95:0.34:0.50), (PNEUMOCOCCAL 13-VALENT CONJUGATE VACCINE:29:10988:0:293:0.97:1.00:0.09:0.17), (WATER:25:10992:0:293:0.97:1.00:0.08:0 0.15), (FULVESTRANT:325:10691:84:210:0.97:0.79:0.61:0.69), (OCTREOTIDE:193:10823:2 7:267:0.97:0.88:0.42:0.57), (DAUNORUBICIN:131:10885:3:291:0.97:0.98:0.31:0.47), (ME THYLCELLULOSE (4000 MPA.S):84:10932:1:293:0.97:0.99:0.22:0.36), (NIMESULIDE:66:10950:5:289:0.97:0.93:0.1 9:0.31), (BUPRENORPHINE HYDROCHLORIDENALOXONE HYDROCHLORIDE:352:10663:165:130:0.97:0.68:0.73:0.70), (INSULIN DEGLUDEC:269:10746:61:234:0.97:0.82:0.53:0.65), (DECITABINE:75:10940:7:288:0.97:0. 91:0.21:0.34), (CLOTIAZEPAM:33:10982:1:294:0.97:0.97:0.10:0.18), (HYDROCHLOROT HIAZIDETELMISARTAN:275:10739:100:196:0.97:0.73:0.58:0.65), (FEXOFENADINEPS EUDOEPHEDRINE:210:10804:50:246:0.97:0.81:0.46:0.59), (PEMETREXED:192:10822:90: 206:0.97:0.68:0.48:0.56), (LURASIDONE HYDROCHLORIDE:190:10824:22:274:0.97:0.90:0.41:0.56), (ICOSAPENT ETHYL:177:10837:18:278:0.97:0.91:0.39:0.54), (TROSPIUM CHLORIDE:220:10793:53:244:0.97:0.81:0.47:0.60), (AMINO ACIDSDEXTROSEELECTROLYTES NOSSOYBEAN OIL:64:10949:10:287:0.97:0.86:0.18:0.30), (ALIROCUMAB:58:10955:2:295:0.97:0.97:0.16: 0.28), (GLUCOSAMINE SULFATE:39:10974:0:297:0.97:1.00:0.12:0.21), (ESTROGENS, CONJUGATEDMEDROXYPROGESTERONE ACETATE:299:10713:127:171:0.97:0.70:0.64:0.67), (CHONDROITIN SULFATE (BOVINE)GLUCOSAMINE HYDROCHLORIDE:277:10735:47:251:0.97:0.85:0.52:0.65), (CHLORAMPHENICOL:86:10 926:9:289:0.97:0.91:0.23:0.37), (TRIHEXYPHENIDYL HYDROCHLORIDE:82:10930:10:288:0.97:0.89:0.22:0.35), (ACEBUTOLOL:55:10957:1:29 7:0.97:0.98:0.16:0.27), (MENTHOL:12:10999:0:299:0.97:1.00:0.04:0.07), (NOREPINEPHR INE:156:10854:62:238:0.97:0.72:0.40:0.51), (PEGINTERFERON BETA-1A:149:10861:246:54:0.97:0.38:0.73:0.50), (PHENTERMINE:71:10939:0:300:0.97:1.00:0.1 9:0.32), (ETHYL LOFLAZEPATE:28:10982:1:299:0.97:0.97:0.09:0.16), (OXALIPLATIN:615:10394:2:299:0.9 7:1.00:0.67:0.80), (FERROUS SULFATEFOLIC ACID:123:10886:22:279:0.97:0.85:0.31:0.45), (BUPRENORPHINE HYDROCHLORIDE:361:10647:153:149:0.97:0.70:0.71:0.71), (AMLODIPINE BESYLATEOLMESARTAN MEDOXOMIL:240:10768:27:275:0.97:0.90:0.47:0.61), (ASPIRINDIPYRIDAMOLE:145:10 863:6:296:0.97:0.96:0.33:0.49), (DOCUSATESENNOSIDES:62:10946:2:300:0.97:0.97:0.1 7:0.29), (ARFORMOTEROL TARTRATE:43:10965:0:302:0.97:1.00:0.12:0.22), (FLUDARABINE PHOSPHATE:478:10529:0:303:0.97:1.00:0.61:0.76), (OSIMERTINIB:122:10885:23:280:0.9 7:0.84:0.30:0.45), (PALONOSETRON:74:10933:2:301:0.97:0.97:0.20:0.33), (ZINC SULFATE\ZINC SULFATE ANHYDROUS:57:10950:3:300:0.97:0.95:0.16:0.27), (25-HYDROXYCHOLECALCIFEROL, 5-TRANS-:43:10964:1:302:0.97:0.98:0.12:0.22), (NEFOPAM:15:10992:0:303:0.97:1.00:0.05: 0.09), (DRONEDARONE:236:10770:39:265:0.97:0.86:0.47:0.61), (MIANSERIN:88:10918:3: 301:0.97:0.97:0.23:0.37), (GLUTAMINE:84:10922:18:286:0.97:0.82:0.23:0.36), (ZONISA MIDE:503:10502:95:210:0.97:0.84:0.71:0.77), (ALBUTEROLIPRATROPIUM:183:10822:2 3:282:0.97:0.89:0.39:0.55), (ATENOLOLCHLORTHALIDONE:129:10876:12:293:0.97:0.9 1:0.31:0.46), (FLURBIPROFEN:107:10898:15:290:0.97:0.88:0.27:0.41), (TRIMEBUTINE:6 0:10945:1:304:0.97:0.98:0.16:0.28), (IRINOTECAN:419:10585:55:251:0.97:0.88:0.63:0.73), (PYRIDOSTIGMINE BROMIDE:202:10802:35:271:0.97:0.85:0.43:0.57), (MEDROXYPROGESTERONE:156:108 48:25:281:0.97:0.86:0.36:0.50), (CICLESONIDE:299:10704:66:241:0.97:0.82:0.55:0.66), (R EGORAFENIB:223:10780:111:196:0.97:0.67:0.53:0.59), (PROCHLORPERAZINE EDISYLATE:66:10937:2:305:0.97:0.97:0.18:0.30), (ACETAMINOPHEN\CAFFEINEPROM ETHAZINESALICYLAMIDE:56:10947:5:302:0.97:0.92:0.16:0.27), (AMANTADINE HYDROCHLORIDE:216:10786:42:266:0.97:0.84:0.45:0.58), (VEMURAFENIB:212:10790:3 9:269:0.97:0.84:0.44:0.58), (OFATUMUMAB:67:10935:4:304:0.97:0.94:0.18:0.30), (PROB ENECID:32:10970:2:306:0.97:0.94:0.09:0.17), (SULFASALAZINE:719:10282:0:309:0.97:1. 00:0.70:0.82), (HERBALSTURMERIC:275:10726:105:204:0.97:0.72:0.57:0.64), (BACITR ACIN:185:10816:39:270:0.97:0.83:0.41:0.54), (TETRAHYDROZOLINE HYDROCHLORIDE:45:10956:0:309:0.97:1.00:0.13:0.23), (MINERAL OIL:41:10960:1:308:0.97:0.98:0.12:0.21), (PIPERACILLIN:123:10877:14:296:0.97:0.90:0.2 9:0.44), (QUINAPRIL HYDROCHLORIDE:117:10883:4:306:0.97:0.97:0.28:0.43), (GLYCERINHYPROMELLOS ESPOLYETHYLENE GLYCOL 400:106:10894:5:305:0.97:0.95:0.26:0.41), (CALCIUM CHLORIDE:12:10988:0:310:0.97:1.00:0.04:0.07), (AMISULPRIDE:187:10812:193:118:0.97: 0.49:0.61:0.55), (ALTEPLASE:373:10625:0:312:0.97:1.00:0.54:0.71), (ESTRONESCROFA OVARY:213:10784:42:271:0.97:0.84:0.44:0.58), (POTASSIUM ASPARTATE:132:10865:0:313:0.97:1.00:0.30:0.46), (CEFPODOXIME:10:10987:0:313:0.97: 1.00:0.03:0.06), (CARBOHYDRATESPOTASSIUM CHLORIDE\SODIUM CHLORIDE\SODIUM LACTATE:76:10920:5:309:0.97:0.94:0.20:0.33), (PNEUMOCOCCAL VACCINE POLYVALENT:65:10931:1:313:0.97:0.98:0.17:0.29), (VOGLIBOSE:164:10831:12:303:0.97: 0.93:0.35:0.51), (NIZATIDINE:62:10933:1:314:0.97:0.98:0.16:0.28), (FORMOTEROL FUMARATE:299:10695:115:201:0.97:0.72:0.60:0.65), (TIGECYCLINE:183:10811:48:268:0. 97:0.79:0.41:0.54), (AMBROXOL:109:10885:10:306:0.97:0.92:0.26:0.41), (VISMODEGIB: 88:10906:16:300:0.97:0.85:0.23:0.36), (HYDROCODONE BITARTRATE:17:10977:0:316:0.97:1.00:0.05:0.10), (CETUXIMAB:296:10697:62:255:0.97: 0.83:0.54:0.65), (IDELALISIB:278:10715:71:246:0.97:0.80:0.53:0.64), (PIROXICAM:252:1 0741:97:220:0.97:0.72:0.53:0.61), (BROMHEXINE HYDROCHLORIDE:58:10935:2:315:0.97:0.97:0.16:0.27), (WHOLE BLOOD:36:10957:0:317:0.97:1.00:0.10:0.19), (NORFLOXACIN:32:10961:0:317:0.97:1.00: 0.09:0.17), (BENAZEPRILBENAZEPRIL HYDROCHLORIDE:267:10725:133:185:0.97:0.67:0.59:0.63), (PEGINTERFERON ALFA-2B:166:10826:28:290:0.97:0.86:0.36:0.51), (BENIDIPINE HYDROCHLORIDE:95:10897:6:312:0.97:0.94:0.23:0.37), (ABIRATERONE ACETATE:268:10722:31:289:0.97:0.90:0.48:0.63), (ERTAPENEM:41:10949:2:318:0.97:0.9 5:0.11:0.20), (DALFAMPRIDINE:525:10464:124:197:0.97:0.81:0.73:0.77), (SODIUM FLUORIDE:196:10793:35:286:0.97:0.85:0.41:0.55), (FLUOROMETHOLONE:142:10847:15: 306:0.97:0.90:0.32:0.47), (HYDROXYCHLOROQUINE DIPHOSPHATE:221:10767:218:104:0.97:0.50:0.68:0.58), (EPINASTINE HYDROCHLORIDE:180:10808:25:297:0.97:0.88:0.38:0.53), (DOPAMINE HYDROCHLORIDE:146:10841:10:313:0.97:0.94:0.32:0.47), (CHONDROITIN SULFATE AGLUCOSAMINE:145:10842:6:317:0.97:0.96:0.31:0.47), (TRIMEBUTINE MALEATE:22:10965:0:323:0.97:1.00:0.06:0.12), (CAPECITABINE:670:10316:3:321:0.97:1 0.00:0.68:0.81), (AMANTADINE:305:10681:74:250:0.97:0.80:0.55:0.65), (MEPERIDINE:24 1:10745:28:296:0.97:0.90:0.45:0.60), (CLEMASTINE:20:10966:0:324:0.97:1.00:0.06:0.11), (LEUCOVORIN:642:10343:0:325:0.97:1.00:0.66:0.80), (ETHINYL ESTRADIOLNORGESTIMATE:230:10754:16:310:0.97:0.93:0.43:0.59), (INSULIN BEEF:189:10795:32:294:0.97:0.86:0.39:0.54), (METHIMAZOLE:389:10594:62:265:0.97:0.8 6:0.59:0.70), (NORTRIPTYLINE HYDROCHLORIDE:271:10712:26:301:0.97:0.91:0.47:0.62), (NADROPARIN CALCIUM:253:10730:48:279:0.97:0.84:0.48:0.61), (HUMAN PLASMA:71:10912:3:324:0.97:0.96:0.18:0.30), (FUSIDIC ACID:210:10772:128:200:0.97:0.62:0.51:0.56), (DICLOFENAC EPOLAMINE:60:10922:1:327:0.97:0.98:0.16:0.27), (TERAZOSIN HYDROCHLORIDE:251:10730:36:293:0.97:0.87:0.46:0.60), (GLYCOPYRRONIUMINDA CATEROL:243:10737:197:133:0.97:0.55:0.65:0.60), (ADO-TRASTUZUMAB EMTANSINE:225:10755:149:181:0.97:0.60:0.55:0.58), (NALTREXONE:210:10770:32:298: 0.97:0.87:0.41:0.56), (AXITINIB:195:10785:48:282:0.97:0.80:0.41:0.54), (LIMAPROST:11 4:10866:4:326:0.97:0.97:0.26:0.41), (RALOXIFENE HYDROCHLORIDE:442:10537:126:205:0.97:0.78:0.68:0.73), (MICAFUNGIN:296:10683:32: 299:0.97:0.90:0.50:0.64), (AZELASTINE:261:10718:42:289:0.97:0.86:0.47:0.61), (CHON DROITIN SULFATE (BOVINE):68:10911:3:328:0.97:0.96:0.17:0.29), (ROFLUMILAST:315:10663:177:155:0.97: 0.64:0.67:0.65), (PENICILLIN V:194:10784:41:291:0.97:0.83:0.40:0.54), (ELDECALCITOL:173:10805:11:321:0.97:0.94:0 0.35:0.51), (GLUTATHIONE:108:10870:1:331:0.97:0.99:0.25:0.39), (MENATETRENONE:8 3:10895:3:329:0.97:0.97:0.20:0.33), (FLURBIPROFEN AXETIL:50:10928:3:329:0.97:0.94:0.13:0.23), (LEVETIRACETAM:1894:9083:0:333:0.97:1 0.00:0.85:0.92), (ISONIAZID:545:10431:9:325:0.97:0.98:0.63:0.77), (LIOTHYRONINE:195: 10781:35:299:0.97:0.85:0.39:0.54), (FONDAPARINUX SODIUM:181:10795:31:303:0.97:0.85:0.37:0.52), (CROMOLYN SODIUM:36:10940:1:333:0.97:0.97:0.10:0.18), (TRIAMTERENE:409:10566:64:271:0.97:0. 86:0.60:0.71), (SEVOFLURANE:140:10835:11:324:0.97:0.93:0.30:0.46), (SIMEPREVIR:11 0:10865:4:331:0.97:0.96:0.25:0.40), (CEFIXIME:42:10932:0:336:0.97:1.00:0.11:0.20), (QU ININE:38:10936:1:335:0.97:0.97:0.10:0.18), (TRASTUZUMAB:491:10482:10:327:0.97:0.98: 0.60:0.74), (SUMATRIPTANSUMATRIPTAN SUCCINATE:384:10589:162:175:0.97:0.70:0.69:0.70), (PANTETHINE:72:10901:2:335:0.97: 0.97:0.18:0.30), (FLUOCINONIDE:269:10703:55:283:0.97:0.83:0.49:0.61), (TETRACYCL INE:220:10752:240:98:0.97:0.48:0.69:0.57), (BENSERAZIDE HYDROCHLORIDELEVODOPA:135:10837:4:334:0.97:0.97:0.29:0.44), (PENICILLIN V POTASSIUM:126:10846:14:324:0.97:0.90:0.28:0.43), (PRASTERONE:125:10847:5:333:0.97: 0.96:0.27:0.43), (SEVELAMER CARBONATE:228:10743:24:315:0.97:0.90:0.42:0.57), (ACLIDINIUM BROMIDE:337:10633:33:307:0.97:0.91:0.52:0.66), (THYROID, PORCINETHYROID, UNSPECIFIED:208:10761:14:327:0.97:0.94:0.39:0.55), (ERTAPENEM SODIUM:105:10864:28:313:0.97:0.79:0.25:0.38), (HYPROMELLOSES:36:10933:0:341:0.97: 1.00:0.10:0.17), (FLUTICASONE FUROATEVILANTEROL TRIFENATATE:554:10414:82:260:0.97:0.87:0.68:0.76), (HYOSCYAMINE:334:10634:122:2 20:0.97:0.73:0.60:0.66), (BIFIDOBACTERIUM SPP.:185:10783:11:331:0.97:0.94:0.36:0.52), (IVABRADINE:144:10824:8:334:0.97:0.95:0.3 0:0.46), (PENICILLIN G:156:10811:58:285:0.97:0.73:0.35:0.48), (AZELASTINE HYDROCHLORIDEFLUTICASONE PROPIONATE:137:10830:10:333:0.97:0.93:0.29:0.44), (LEVOTHYROXINELIOTHYRONI NE:111:10856:5:338:0.97:0.96:0.25:0.39), (ESTAZOLAM:81:10886:4:339:0.97:0.95:0.19:0 0.32), (SODIUM ALGINATE:67:10900:1:342:0.97:0.99:0.16:0.28), (AZILSARTAN KAMEDOXOMIL:257:10709:30:314:0.97:0.90:0.45:0.60), (MEGESTROL:173:10793:22:322: 0.97:0.89:0.35:0.50), (DOXORUBICIN:1121:9844:0:345:0.97:1.00:0.76:0.87), (DIPYRON E:244:10721:78:267:0.97:0.76:0.48:0.59), (RASAGILINE MESYLATE:107:10858:5:340:0.97:0.96:0.24:0.38), (AMILORIDE:61:10904:1:344:0.97:0.9 8:0.15:0.26), (NITROFURANTOINNITROFURANTOIN MONOHYDRATE:306:10658:37:309:0.97:0.89:0.50:0.64), (CEFEPIMECEFEPIME HYDROCHLORIDE:160:10804:29:317:0.97:0.85:0.34:0.48), (HYDROXOCOBALAMIN:68: 10896:5:341:0.97:0.93:0.17:0.28), (KETOTIFEN FUMARATE:35:10929:0:346:0.97:1.00:0.09:0.17), (PHENYTOIN SODIUM:272:10691:66:281:0.97:0.80:0.49:0.61), (PROPAFENONE:197:10766:20:327:0.97: 0.91:0.38:0.53), (QUININE SULFATE:57:10906:0:347:0.97:1.00:0.14:0.25), (PRAZOSIN:279:10683:45:303:0.97:0.86:0 0.48:0.62), (TRICHLORMETHIAZIDE:176:10786:13:335:0.97:0.93:0.34:0.50), (BISOPROLOL FUMARATEHYDROCHLOROTHIAZIDE:136:10826:3:345:0.97:0.98:0.28:0.44), (NINTED ANIB:273:10688:91:258:0.97:0.75:0.51:0.61), (GLUCAGON:143:10818:8:341:0.97:0.95:0.3 0:0.45), (DEXAMETHASONETOBRAMYCIN:124:10837:6:343:0.97:0.95:0.27:0.42), (BE NZYDAMINE HYDROCHLORIDE:35:10926:0:349:0.97:1.00:0.09:0.17), (ZINC:333:10627:95:255:0.97:0. 78:0.57:0.66), (HYALURONATE SODIUM:285:10675:31:319:0.97:0.90:0.47:0.62), (DESONIDE:244:10716:47:303:0.97:0.84: 0.45:0.58), (CORTICOTROPIN:169:10790:141:210:0.97:0.55:0.45:0.49), (ROTIGOTINE:11 7:10842:2:349:0.97:0.98:0.25:0.40), (VALGANCICLOVIR HYDROCHLORIDE:431:10527:126:226:0.97:0.77:0.66:0.71), (PREDNISOLONE ACETATE:376:10582:56:296:0.97:0.87:0.56:0.68), (PEMETREXED DISODIUM:251:10707:126:226:0.97:0.67:0.53:0.59), (MICONAZOLE NITRATE:203:10755:21:331:0.97:0.91:0.38:0.54), (DURVALUMAB:97:10861:3:349:0.97:0 0.97:0.22:0.36), (EBASTINE:35:10923:0:352:0.97:1.00:0.09:0.17), (UMECLIDINIUM BROMIDEVILANTEROL TRIFENATATE:327:10630:248:105:0.97:0.57:0.76:0.65), (LERCANIDIPINE HYDROCHLORIDE:318:10639:197:156:0.97:0.62:0.67:0.64), (TIMOLOL MALEATE:253:10704:31:322:0.97:0.89:0.44:0.59), (VASOPRESSIN:139:10817:23:331:0.97: 0.86:0.30:0.44), (FOSAPREPITANT DIMEGLUMINE:33:10922:3:352:0.97:0.92:0.09:0.16), (TERBINAFINE HYDROCHLORIDE:277:10677:81:275:0.97:0.77:0.50:0.61), (HEPARIN CALCIUM:220:10734:56:300:0.97:0.80:0.42:0.55), (BUDESONIDEFORMOTEROL FUMARATE:271:10682:168:189:0.97:0.62:0.59:0.60), (OXYMETAZOLINE:156:10797:16:3 41:0.97:0.91:0.31:0.47), (NALOXEGOL OXALATE:97:10856:4:353:0.97:0.96:0.22:0.35), (DOFETILIDE:315:10637:145:213:0.97:0. 68:0.60:0.64), (CANAKINUMAB:207:10745:167:191:0.97:0.55:0.52:0.54), (GLYCOSAMI NOGLYCANS:144:10807:10:349:0.97:0.94:0.29:0.45), (BRIMONIDINE TARTRATETIMOLOL MALEATE:182:10768:15:345:0.97:0.92:0.35:0.50), (ETHINYL ESTRADIOLNORETHINDRONE ACETATE:133:10817:2:358:0.97:0.99:0.27:0.42), (FLUDROCORTISONE ACETATE:266:10683:81:280:0.97:0.77:0.49:0.60), (ESOMEPRAZOLE SODIUM:95:10854:9:352:0.97:0.91:0.21:0.34), (ALCOHOL:279:10669:173:189:0.97:0.62:0 0.60:0.61), (EDOXABAN TOSYLATE:260:10688:197:165:0.97:0.57:0.61:0.59), (RIZATRIPTAN BENZOATE:371:10576:139:224:0.97:0.73:0.62:0.67), (TIOTROPIUM:331:10616:115:248:0. 97:0.74:0.57:0.65), (AMIKACIN SULFATE:157:10790:23:340:0.97:0.87:0.32:0.46), (LEFLUNOMIDE:656:10290:10:354:0.97: 0.98:0.65:0.78), (INDAPAMIDEPERINDOPRIL:248:10698:267:97:0.97:0.48:0.72:0.58), (TOCILIZUMAB:598:10346:163:203:0.97:0.79:0.75:0.77), (VINCRISTINE SULFATE:439:10505:58:308:0.97:0.88:0.59:0.71), (EPIRUBICIN:277:10667:89:277:0.97:0. 76:0.50:0.60), (SAXAGLIPTIN HYDROCHLORIDE:264:10680:7:359:0.97:0.97:0.42:0.59), (BENDROFLUMETHIAZIDE:1 65:10779:4:362:0.97:0.98:0.31:0.47), (DESMOPRESSIN:126:10818:18:348:0.97:0.88:0.27: 0.41), (ATROPINE SULFATE:124:10819:33:334:0.97:0.79:0.27:0.40), (COBIMETINIB:35:10908:0:367:0.97:1. 00:0.09:0.16), (BRINZOLAMIDE:314:10628:87:281:0.97:0.78:0.53:0.63), (PAMIDRONATE DISODIUM:295:10647:59:309:0.97:0.83:0.49:0.62), (.ALPHA.-TOCOPHEROL ACETATE, DL-ASCORBIC ACIDCYANOCOBALAMINFLUORIDE IONFOLIC ACIDNIACINPYRIDOXINE\RIBOFLAVINTHIAMINEVITAMIN AVITAMIN D:159:10783:27:341:0.97:0.85:0.32:0.46), (CINACALCET HYDROCHLORIDE:320:10621:44:325:0.97:0.88:0.50:0.63), (MECLIZINEMECLIZINE HYDROCHLORIDE:279:10662:37:332:0.97:0.88:0.46:0.60), (ACETAMINOPHENDIPHEN HYDRAMINE:164:10777:9:360:0.97:0.95:0.31:0.47), (SAW PALMETTO:138:10803:3:366:0.97:0.98:0.27:0.43), (CLEMASTINE FUMARATE:99:10842:9:360:0.97:0.92:0.22:0.35), (CEFOPERAZONE SODIUMSULBACTAM SODIUM:53:10888:1:368:0.97:0.98:0.13:0.22), (VORTIOXETINE HYDROBROMIDE:198:10742:8:362:0.97:0.96:0.35:0.52), (EXENATIDE:588:10351:1:370:0 0.97:1.00:0.61:0.76), (PIOGLITAZONE:219:10720:17:354:0.97:0.93:0.38:0.54), (ACARBOS E:321:10617:127:245:0.97:0.72:0.57:0.63), (PONATINIB:316:10622:204:168:0.97:0.61:0.65: 0.63), (RIVASTIGMINE:246:10692:60:312:0.97:0.80:0.44:0.57), (NICARDIPINE:209:1072 8:37:336:0.97:0.85:0.38:0.53), (FLECAINIDE ACETATE:95:10842:4:369:0.97:0.96:0.20:0.34), (LACOSAMIDE:469:10467:56:318:0.97:0. 89:0.60:0.71), (SECUKINUMAB:431:10505:251:123:0.97:0.63:0.78:0.70), (FESOTERODINE FUMARATE:370:10566:144:230:0.97:0.72:0.62:0.66), (PALIPERIDONE:88:10848:0:374:0.9 7:1.00:0.19:0.32), (TOFACITINIB CITRATE:610:10325:91:284:0.97:0.87:0.68:0.76), (PENTOSAN POLYSULFATE SODIUM:267:10668:49:326:0.97:0.84:0.45:0.59), (DIOSMINHESPERIDIN:42:10893:1:374: 0.97:0.98:0.10:0.18), (LITHIUM CARBONATE:539:10395:134:242:0.97:0.80:0.69:0.74), (CRIZOTINIB:263:10671:53:323:0. 97:0.83:0.45:0.58), (CHONDROITIN SULFATE (BOVINE)GLUCOSAMINE SULFATE:84:10850:0:376:0.97:1.00:0.18:0.31), (TOCOPHEROL:245:10688:50:327:0.97:0. 83:0.43:0.57), (DORZOLAMIDE HYDROCHLORIDETIMOLOL MALEATE:164:10769:3:374:0.97:0.98:0.30:0.47), (CARBAZOCHROME SODIUM SULFONATE:106:10827:8:369:0.97:0.93:0.22:0.36), (IODIXANOL:8:10925:0:377:0.97:1.0 0:0.02:0.04), (SACCHAROMYCES CEREVISIAE:228:10704:38:340:0.97:0.86:0.40:0.55), (AZOSEMIDE:192:10740:13:365:0.97: 0.94:0.34:0.50), (TRIMETAZIDINE DIHYDROCHLORIDE:58:10874:1:377:0.97:0.98:0.13:0.23), (CARVEDILOL PHOSPHATE:195:10736:322:57:0.97:0.38:0.77:0.51), (BEZAFIBRATE:88:10843:3:376:0.97: 0.97:0.19:0.32), (RITUXIMAB:1557:9373:0:380:0.97:1.00:0.80:0.89), (METAXALONE:45 9:10471:178:202:0.97:0.72:0.69:0.71), (DEXTROMETHORPHAN:6:10924:0:380:0.97:1.00: 0.02:0.03), (AZTREONAM:131:10798:8:373:0.97:0.94:0.26:0.41), (DEVICE:482:10446:188: 194:0.97:0.72:0.71:0.72), (TERBUTALINE SULFATE:306:10622:116:266:0.97:0.73:0.53:0.62), (DAPSONE:266:10661:19:364:0.97:0.9 3:0.42:0.58), (CLOBAZAM:366:10559:53:332:0.97:0.87:0.52:0.66), (ACETAMINOPHEN OXYCODONE HYDROCHLORIDEOXYCODONE TEREPHTHALATE:243:10682:39:346:0.97:0.86:0.41:0.56), (MEMANTINE:108:10817:2:38 3:0.97:0.98:0.22:0.36), (OXYMORPHONE HYDROCHLORIDE:41:10884:0:385:0.97:1.00:0.10:0.18), (PERTUZUMAB:197:10727:37:3 49:0.97:0.84:0.36:0.51), (DACLIZUMAB:133:10790:332:55:0.97:0.29:0.71:0.41), (SILVER SULFADIAZINE:162:10760:17:371:0.97:0.91:0.30:0.46), (TERBINAFINE:89:10833:1:387:0 0.97:0.99:0.19:0.31), (CALCIUM ACETATE:403:10518:170:219:0.97:0.70:0.65:0.67), (ALENDRONIC ACID:329:10592:127:262:0.97:0.72:0.56:0.63), (DALTEPARIN:270:10651:72:317:0.97:0.79: 0.46:0.58), (PSYLLIUM HUSK:260:10659:24:367:0.97:0.92:0.41:0.57), (INSULIN GLULISINE:302:10616:47:345:0.97:0.87:0.47:0.61), (ALIZAPRIDE HYDROCHLORIDE:5:10913:0:392:0.97:1.00:0.01:0.02), (HYDROCHLOROTHIAZIDEIR BESARTAN:403:10514:119:274:0.97:0.77:0.60:0.67), (ERLOTINIB:163:10754:9:384:0.97:0 0.95:0.30:0.45), (CALCIUM POLYSTYRENE SULFONATE:78:10839:1:392:0.97:0.99:0.17:0.28), (CYTARABINE:715:10201:1:393:0.97: 1.00:0.65:0.78), (DIPYRIDAMOLE:263:10653:45:349:0.97:0.85:0.43:0.57), (DIFLUPREDN ATE:121:10795:4:390:0.97:0.97:0.24:0.38), (PENTAMIDINE:120:10796:7:387:0.97:0.94:0. 24:0.38), (MEPERIDINE HYDROCHLORIDE:232:10683:14:381:0.97:0.94:0.38:0.54), (CARBOXYMETHYLCELLU LOSE SODIUM:209:10706:12:383:0.97:0.95:0.35:0.51), (DROXIDOPA:110:10805:17:378:0.97:0.8 7:0.23:0.36), (GARLIC:201:10713:7:389:0.96:0.97:0.34:0.50), (FAMCICLOVIR:116:10798: 5:391:0.96:0.96:0.23:0.37), (SUVOREXANT:83:10831:3:393:0.96:0.97:0.17:0.30), (DIMET HICONELOPERAMIDE HYDROCHLORIDE:6:10908:0:396:0.96:1.00:0.01:0.03), (CANDESARTAN CILEXETILHYDROCHLOROTHIAZIDE:392:10519:247:152:0.96:0.61:0.72:0.66), (PIOGL ITAZONE HYDROCHLORIDE:442:10468:4:396:0.96:0.99:0.53:0.69), (NABUMETONE:329:10581:61: 339:0.96:0.84:0.49:0.62), (IMIPENEM:186:10724:60:340:0.96:0.76:0.35:0.48), (TESTOST ERONE CYPIONATE:223:10686:12:389:0.96:0.95:0.36:0.53), (MICONAZOLE:62:10844:0:404:0.96: 1.00:0.13:0.23), (LEVOMEPROMAZINE:92:10813:2:403:0.96:0.98:0.19:0.31), (ALUMIN UM HYDROXIDEDIMETHICONEMAGNESIUM HYDROXIDE:268:10636:36:370:0.96:0.88:0.42:0.57), (AMINO ACIDSDEXTROSEELECTROLYTES NOSVITAMINS:29:10874:0:407:0.96:1.00:0.07:0.12), (NEFOPAM HYDROCHLORIDE:93:10809:1:407:0.96:0.99:0.19:0.31), (HYDROXYZINE PAMOATE:485:10416:145:264:0.96:0.77:0.65:0.70), (ELETRIPTAN HYDROBROMIDE:345:10556:250:159:0.96:0.58:0.68:0.63), (LITHIUM:331:10570:26:383:0 0.96:0.93:0.46:0.62), (CEFUROXIME AXETIL:228:10673:23:386:0.96:0.91:0.37:0.53), (POLYETHYLENE GLYCOL 4000:186:10714:29:381:0.96:0.87:0.33:0.48), (NADOLOL:376:10523:77:334:0.96:0.83:0.53: 0.65), (CYANOCOBALAMINPYRIDOXINE HYDROCHLORIDETHIAMINE:34:10864:0:412:0.96:1.00:0.08:0.14), (TINZAPARIN SODIUM:357:10540:65:348:0.96:0.85:0.51:0.63), (LISDEXAMFETAMINE DIMESYLATE:480:10416:152:262:0.96:0.76:0.65:0.70), (CARFILZOMIB:410:10486:153:26 1:0.96:0.73:0.61:0.66), (VARENICLINE TARTRATE:543:10351:201:215:0.96:0.73:0.72:0.72), (FLUVASTATIN SODIUM:229:10665:13:403:0.96:0.95:0.36:0.52), (KETAMINE:105:10788:4:413:0.96:0.96: 0.20:0.33), (IPRATROPIUM:479:10413:110:308:0.96:0.81:0.61:0.70), (AFATINIB:162:1073 0:12:406:0.96:0.93:0.29:0.44), (DEXPANTHENOL:46:10846:1:417:0.96:0.98:0.10:0.18), (T IMOLOL:473:10418:107:312:0.96:0.82:0.60:0.69), (COLESEVELAM HYDROCHLORIDE:366:10524:30:390:0.96:0.92:0.48:0.64), (SOFOSBUVIR:231:10659:0:4 20:0.96:1.00:0.35:0.52), (TIOTROPIUM BROMIDE:163:10727:4:416:0.96:0.98:0.28:0.44), (BENAZEPRIL HYDROCHLORIDE:532:10357:74:347:0.96:0.88:0.61:0.72), (VITAMIN A:295:10594:66:355:0.96:0.82:0.45:0.58), (RACECADOTRIL:52:10837:1:420:0.96:0.98:0.1 1:0.20), (CLOZAPINE:570:10318:145:277:0.96:0.80:0.67:0.73), (PANCRELIPASE:254:106 34:17:405:0.96:0.94:0.39:0.55), (MILK THISTLE:175:10713:8:414:0.96:0.96:0.30:0.45), (FERROUS CITRATE:164:10724:7:415:0.96:0.96:0.28:0.44), (BUTYLSCOPOLAMINE BROMIDE:306:10581:53:370:0.96:0.85:0.45:0.59), (PANTOPRAZOLE MAGNESIUM:288:10599:327:96:0.96:0.47:0.75:0.58), (GLYCOPYRROLATE:242:10645:53: 370:0.96:0.82:0.40:0.53), (CHLORPROMAZINE:241:10646:24:399:0.96:0.91:0.38:0.53), (CANNABIS SATIVA SUBSP. INDICA TOP:189:10698:10:413:0.96:0.95:0.31:0.47), (PHENAZOPYRIDINE:127:10760:4:419:0.96: 0.97:0.23:0.38), (MOMETASONE:123:10764:4:419:0.96:0.97:0.23:0.37), (TERIFLUNOMI DE:389:10497:330:94:0.96:0.54:0.81:0.65), (DOCUSATE CALCIUM:372:10514:50:374:0.96:0.88:0.50:0.64), (KETOROLACKETOROLAC TROMETHAMINE:259:10626:34:391:0.96:0.88:0.40:0.55), (DICLOFENAC POTASSIUM:71:10814:0:425:0.96:1.00:0.14:0.25), (SITAGLIPTIN:312:10572:52:374:0.96: 0.86:0.45:0.59), (PHENYLEPHRINEPHENYLEPHRINE HYDROCHLORIDE:106:10777:1:426:0.96:0.99:0.20:0.33), (SODIUM PHOSPHATE:40:10843:0:427:0.96:1.00:0.09:0.16), (ZINC OXIDE:69:10813:1:427:0.96:0.99:0.14:0.24), (IRON SUCROSE:250:10631:25:404:0.96:0.91:0.38:0.54), (LORMETAZEPAM:183:10698:5:424:0. 96:0.97:0.30:0.46), (EPLERENONE:362:10518:82:348:0.96:0.82:0.51:0.63), (PENTOXIFY LLINE:261:10619:22:408:0.96:0.92:0.39:0.55), (HYOSCYAMINE SULFATE:187:10692:6:425:0.96:0.97:0.31:0.46), (NITRAZEPAM:70:10809:0:431:0.96:1.00: 0.14:0.25), (PENICILLIN:379:10499:166:266:0.96:0.70:0.59:0.64), (PRIMIDONE:483:1039 4:193:240:0.96:0.71:0.67:0.69), (CALCIUM POLYCARBOPHIL:214:10663:15:418:0.96:0.93:0.34:0.50), (HYDROCORTISONE BUTYRATE:63:10814:0:433:0.96:1.00:0.13:0.23), (FLUTICASONE FUROATESALMETEROL XINAFOATE:352:10524:91:343:0.96:0.79:0.51:0.62), (MUPIROCINMUPIROCIN CALCIUM:285:10591:45:389:0.96:0.86:0.42:0.57), (MAGNESIUM CHLORIDE:66:10810:0:434:0.96:1.00:0.13:0.23), (AMINO ACIDS:90:10785:0:435:0.96:1.00:0.17:0.29), (CANAGLIFLOZIN:424:10449:23:414:0.96:0. 95:0.51:0.66), (SUMATRIPTAN:401:10472:74:363:0.96:0.84:0.52:0.65), (MESNA:271:106 02:43:394:0.96:0.86:0.41:0.55), (ATOVAQUONE:262:10611:44:393:0.96:0.86:0.40:0.55), (SULPIRIDE:106:10767:4:433:0.96:0.96:0.20:0.33), (PHLOROGLUCINOL:77:10796:1:436:0 0.96:0.99:0.15:0.26), (ACENOCOUMAROL:354:10518:51:387:0.96:0.87:0.48:0.62), (LENO GRASTIM:187:10685:8:430:0.96:0.96:0.30:0.46), (LIOTHYRONINE SODIUM:274:10597:38:401:0.96:0.88:0.41:0.56), (PHENPROCOUMON:212:10659:19:420:0 0.96:0.92:0.34:0.49), (ARMODAFINIL:475:10395:138:302:0.96:0.77:0.61:0.68), (LEDIPAS VIRSOFOSBUVIR:412:10458:240:200:0.96:0.63:0.67:0.65), (IMATINIB MESYLATE:413:10456:175:266:0.96:0.70:0.61:0.65), (BENDAMUSTINE:299:10570:14:427: 0.96:0.96:0.41:0.58), (MIDODRINE:327:10540:66:377:0.96:0.83:0.46:0.60), (DOPAMINE DOPAMINE HYDROCHLORIDE:118:10748:6:438:0.96:0.95:0.21:0.35), (ISOSORBIDE:508:10357:118:3 27:0.96:0.81:0.61:0.70), (BISACODYL OR DOCUSATE SODIUM:496:10369:138:307:0.96:0.78:0.62:0.69), (PITAVASTATIN CALCIUM:409:10455:51:395:0.96:0.89:0.51:0.65), (PYRIDOXINE:409:10455:67:379:0.96: 0.86:0.52:0.65), (DRONABINOL:358:10506:105:341:0.96:0.77:0.51:0.62), (CEFOTAXIME SODIUM:273:10591:82:364:0.96:0.77:0.43:0.55), (FERROUS GLUCONATE:212:10652:15:431:0.96:0.93:0.33:0.49), (PHENYTOIN:748:10115:43:404:0.9 6:0.95:0.65:0.77), (METHYLPREDNISOLONE ACETATE:300:10563:254:193:0.96:0.54:0.61:0.57), (AMINO ACIDSDEXTROSEELECTROLYTES NOSSOYBEAN OILVITAMINS:213:10650:10:437:0.96:0.96:0.33:0.49), (BETAHISTINE:39:10824:0:447:0. 96:1.00:0.08:0.15), (FLUOROURACIL:852:10010:0:448:0.96:1.00:0.66:0.79), (HUMAN PLATELET, ALLOGENIC:133:10729:5:443:0.96:0.96:0.23:0.37), (MEDROXYPROGESTERONE ACETATE:320:10541:15:434:0.96:0.96:0.42:0.59), (RABEPRAZOLE:426:10434:97:353:0.9 6:0.81:0.55:0.65), (FLUTICASONE FUROATE:339:10521:79:371:0.96:0.81:0.48:0.60), (TRETINOIN:131:10729:6:444:0.96:0.9 6:0.23:0.37), (POTASSIUM GLUCONATE:78:10782:1:449:0.96:0.99:0.15:0.26), (DARATUMUMAB:204:10654:22:430: 0.96:0.90:0.32:0.47), (OSELTAMIVIR PHOSPHATE:244:10613:20:433:0.96:0.92:0.36:0.52), (URAPIDIL:197:10660:11:442:0.96:0 0.95:0.31:0.47), (FLUCLOXACILLIN:95:10762:4:449:0.96:0.96:0.17:0.30), (SODIUM GUALENATE:50:10806:0:454:0.96:1.00:0.10:0.18), (HYDROXYUREA:480:10375:61:394:0 0.96:0.89:0.55:0.68), (DEXTROMETHORPHAN HYDROBROMIDE:72:10783:1:454:0.96:0.99:0.14:0.24), (METOLAZONE:395:10459:35:42 1:0.96:0.92:0.48:0.63), (LEVOCETIRIZINE:143:10711:2:454:0.96:0.99:0.24:0.39), (ILOPR OST:158:10695:408:49:0.96:0.28:0.76:0.41), (CINNAMON:119:10733:0:458:0.96:1.00:0.21: 0.34), (RISEDRONATE SODIUM:632:10219:90:369:0.96:0.88:0.63:0.73), (PAROXETINEPAROXETINE HYDROCHLORIDE:519:10332:110:349:0.96:0.83:0.60:0.69), (SUMATRIPTAN SUCCINATE:424:10427:15:444:0.96:0.97:0.49:0.65), (ACETAZOLAMIDE:368:10482:86:37 4:0.96:0.81:0.50:0.62), (OMEPRAZOLE SODIUM:259:10591:36:424:0.96:0.88:0.38:0.53), (LORATADINEPSEUDOEPHEDRINE SULFATE:98:10752:1:459:0.96:0.99:0.18:0.30), (TOLTERODINE TARTRATE:411:10438:54:407:0.96:0.88:0.50:0.64), (DOCUSATE SODIUMSENNOSIDES:112:10737:1:460:0.96:0.99:0.20:0.33), (TICAGRELOR:608:10240: 12:450:0.96:0.98:0.57:0.72), (CHLORTHALIDONE:397:10451:43:419:0.96:0.90:0.49:0.63), (PEGINTERFERON ALFA-2A:166:10682:4:458:0.96:0.98:0.27:0.42), (RIBOFLAVIN:92:10756:0:462:0.96:1.00:0.17:0. 28), (CAFFEINE:54:10793:1:462:0.96:0.98:0.10:0.19), (GRANULOCYTE COLONY-STIMULATING FACTOR NOS:222:10623:7:458:0.96:0.97:0.33:0.49), (LYSINE:81:10764:0:465:0.96:1.00:0.15:0.26), (LERCANIDIPINE:490:10354:183:283:0.96:0.73:0.63:0.68), (LUTEIN:205:10639:8:458:0.9 6:0.96:0.31:0.47), (CALCIUM CHLORIDE\POTASSIUM CHLORIDE\SODIUM CHLORIDE\SODIUM LACTATE:65:10778:0:467:0.96:1.00:0.12:0.22), (DONEPEZIL:289:10553:15:453:0.96:0.95: 0.39:0.55), (MINOCYCLINE HYDROCHLORIDE:280:10562:29:439:0.96:0.91:0.39:0.54), (DROSPIRENONEETHINYL ESTRADIOL:226:10616:7:461:0.96:0.97:0.33:0.49), (SULFAMETHOXAZOLE:384:10456:1 33:337:0.96:0.74:0.53:0.62), (MILNACIPRAN HYDROCHLORIDE:67:10773:0:470:0.96:1.00:0.12:0.22), (PREDNISOLONE:2641:8198:0:4 71:0.96:1.00:0.85:0.92), (DOXEPIN:319:10520:27:444:0.96:0.92:0.42:0.58), (FLUNITRAZ EPAM:232:10606:5:467:0.96:0.98:0.33:0.50), (PRAMIPEXOLE:517:10319:69:405:0.96:0.88: 0.56:0.69), (LIDOCAINEPRILOCAINE:421:10415:93:381:0.96:0.82:0.52:0.64), (FORMOT EROL FUMARATE DIHYDRATE\MOMETASONE FUROATE:391:10445:59:415:0.96:0.87:0.49:0.62), (THYROID, PORCINE:336:10500:33:441:0.96:0.91:0.43:0.59), (CLINDAMYCIN HYDROCHLORIDE:175:10661:13:461:0.96:0.93:0.28:0.42), (FERROUS FUMARATE:273:10562:23:452:0.96:0.92:0.38:0.53), (MORPHINE HYDROCHLORIDE:101:10734:3:472:0.96:0.97:0.18:0.30), (UREA:75:10759:2:474:0.96:0.9 7:0.14:0.24), (PAZOPANIB HYDROCHLORIDE:331:10502:191:286:0.96:0.63:0.54:0.58), (ERLOTINIB HYDROCHLORIDE:70:10762:2:476:0.96:0.97:0.13:0.23), (FLUINDIONE:220:10611:26:453: 0.96:0.89:0.33:0.48), (EZETIMIBESIMVASTATIN:476:10354:64:416:0.96:0.88:0.53:0.66), (SELENIUM:157:10673:0:480:0.96:1.00:0.25:0.40), (SOTALOL:580:10249:129:352:0.96:0 0.82:0.62:0.71), (RANOLAZINE:538:10291:259:222:0.96:0.68:0.71:0.69), (OMEGA-3-ACID ETHYL ESTERS:506:10323:105:376:0.96:0.83:0.57:0.68), (AMLODIPINE BESYLATEBENAZEPRIL HYDROCHLORIDE:179:10650:5:476:0.96:0.97:0.27:0.43), (EXEMESTANE:331:10496:48: 435:0.96:0.87:0.43:0.58), (AMLODIPINE BESYLATEVALSARTAN:231:10596:7:476:0.96:0.97:0.33:0.49), (POSACONAZOLE:319: 10507:39:445:0.96:0.89:0.42:0.57), (PETROLATUM:160:10666:3:481:0.96:0.98:0.25:0.40), (DEXTROMETHORPHAN HYDROBROMIDEGUAIFENESIN:94:10731:1:484:0.96:0.99:0.16:0.28), (CIMETIDINE:38 0:10444:83:403:0.96:0.82:0.49:0.61), (CALCIUM CITRATE:507:10314:67:422:0.96:0.88:0.55:0.67), (DESVENLAFAXINE SUCCINATE:401:10420:28:461:0.96:0.93:0.47:0.62), (DIMENHYDRINATE:383:10438:88:4 01:0.96:0.81:0.49:0.61), (NALOXONE HYDROCHLORIDEOXYCODONE HYDROCHLORIDE:382:10439:144:345:0.96:0.73:0.53:0.61), (CHLORHEXIDINE:247:1057 4:22:467:0.96:0.92:0.35:0.50), (THALIDOMIDE:464:10356:185:305:0.96:0.71:0.60:0.65), (SCOPOLAMINE:398:10422:96:394:0.96:0.81:0.50:0.62), (CILASTATIN SODIUMIMIPENEM:340:10480:99:391:0.96:0.77:0.47:0.58), (AMPICILLIN:329:10491:32: 458:0.96:0.91:0.42:0.57), (SODIUM POLYSTYRENE SULFONATE:174:10646:6:484:0.96:0.97:0.26:0.42), (ACETAMINOPHENBUTALBITAL CAFFEINE:511:10308:91:400:0.96:0.85:0.56:0.68), (PRAMIPEXOLE DIHYDROCHLORIDE:536:10281:141:352:0.96:0.79:0.60:0.68), (CYCLIZINE:36:10781:0:4 93:0.96:1.00:0.07:0.13), (FLUDROCORTISONE:184:10630:8:488:0.96:0.96:0.27:0.43), (C LINDAMYCIN PHOSPHATE:228:10585:18:479:0.96:0.93:0.32:0.48), (RIFAMPIN:530:10282:13:485:0.96:0 0.98:0.52:0.68), (DIVALPROEX SODIUM:858:9953:139:360:0.96:0.86:0.70:0.77), (DOXAZOSINDOXAZOSIN MESYLATE:550:10260:60:440:0.96:0.90:0.56:0.69), (NICARDIPINE HYDROCHLORIDE:153:10656:7:494:0.96:0.96:0.24:0.38), (FLECAINIDE:470:10337:167:3 36:0.96:0.74:0.58:0.65), (LEVOCARNITINE:141:10666:1:502:0.96:0.99:0.22:0.36), (CEFA ZOLIN SODIUM:274:10532:24:480:0.96:0.92:0.36:0.52), (GINKGO:94:10712:0:504:0.96:1.00:0.16: 0.27), (MAGNESIUM CITRATE:89:10717:0:504:0.96:1.00:0.15:0.26), (ROPINIROLE:397:10408:19:486:0.96:0.95: 0.45:0.61), (AZACITIDINE:238:10567:7:498:0.96:0.97:0.32:0.49), (POVIDONE-IODINE:123:10682:0:505:0.96:1.00:0.20:0.33), (NEBIVOLOL:653:10149:163:345:0.96:0.80: 0.65:0.72), (LEVALBUTEROL HYDROCHLORIDE:448:10353:114:395:0.95:0.80:0.53:0.64), (DASATINIB:333:10468:364: 145:0.95:0.48:0.70:0.57), (MONTMORILLONITE:96:10704:1:509:0.95:0.99:0.16:0.27), (L OVASTATIN:860:9939:138:373:0.95:0.86:0.70:0.77), (CAPTOPRIL:344:10455:51:460:0.95: 0.87:0.43:0.57), (GLYCERIN:194:10604:9:503:0.95:0.96:0.28:0.43), (FELODIPINE:221:10 574:5:510:0.95:0.98:0.30:0.46), (TOLVAPTAN:114:10681:6:509:0.95:0.95:0.18:0.31), (.AL PHA.-LIPOIC ACID:76:10718:0:516:0.95:1.00:0.13:0.23), (FLUOXETINEFLUOXETINE HYDROCHLORIDE:477:10316:62:455:0.95:0.88:0.51:0.65), (CARISOPRODOL:451:10342: 33:484:0.95:0.93:0.48:0.64), (HYDROXYCHLOROQUINE SULFATE:833:9959:27:491:0.95:0.97:0.63:0.76), (MINOCYCLINEMINOCYCLINE HYDROCHLORIDE:377:10415:62:456:0.95:0.86:0.45:0.59), (PHENOBARBITAL:576:1021 4:128:392:0.95:0.82:0.60:0.69), (OCTREOTIDE ACETATE:454:10336:17:503:0.95:0.96:0.47:0.64), (DUTASTERIDE:534:10255:102:419:0.9 5:0.84:0.56:0.67), (SACUBITRILVALSARTAN:407:10382:402:119:0.95:0.50:0.77:0.61), ( GENTAMICIN SULFATE:109:10680:4:517:0.95:0.96:0.17:0.29), (FLUTICASONE:492:10296:117:405:0.95: 0.81:0.55:0.65), (CLONIDINE HYDROCHLORIDE:326:10462:20:502:0.95:0.94:0.39:0.56), (CISPLATIN:1005:9782:1:522: 0.95:1.00:0.66:0.79), (NICORANDIL:233:10553:11:513:0.95:0.95:0.31:0.47), (ESZOPICL ONE:597:10187:178:348:0.95:0.77:0.63:0.69), (AMPICILLIN SODIUMSULBACTAM SODIUM:271:10513:26:500:0.95:0.91:0.35:0.51), (ITRACONAZOLE:337:10446:31:496:0.95: 0.92:0.40:0.56), (TRAVOPROST:214:10569:4:523:0.95:0.98:0.29:0.45), (VILDAGLIPTIN: 171:10612:2:525:0.95:0.99:0.25:0.39), (CHLORPHENIRAMINE:48:10735:0:527:0.95:1.00: 0.08:0.15), (CEFAZOLIN:293:10489:35:493:0.95:0.89:0.37:0.53), (PAROXETINE:566:1021 5:114:415:0.95:0.83:0.58:0.68), (BETAMETHASONE DIPROPIONATE:240:10541:19:510:0.95:0.93:0.32:0.48), (CHLORHEXIDINE GLUCONATE:185:10596:7:522:0.95:0.96:0.26:0.41), (LABETALOLLABETALOL HYDROCHLORIDE:505:10275:125:405:0.95:0.80:0.55:0.66), (LEUCOVORIN CALCIUM:388:10390:43:489:0.95:0.90:0.44:0.59), (CODEINE PHOSPHATE:204:10574:16:516:0.95:0.93:0.28:0.43), (TURMERIC:113:10665:2:530:0.95:0 0.98:0.18:0.30), (CORTISONEHYDROCORTISONE:541:10236:141:392:0.95:0.79:0.58:0.6 7), (FLUOXETINE HYDROCHLORIDE:1590:9186:0:534:0.95:1.00:0.75:0.86), (DESLORATADINE:452:10324: 89:445:0.95:0.84:0.50:0.63), (TIZANIDINE HYDROCHLORIDE:623:10151:103:433:0.95:0.86:0.59:0.70), (CHOLESTYRAMINE:536:10 237:181:356:0.95:0.75:0.60:0.67), (MODAFINIL:487:10286:45:492:0.95:0.92:0.50:0.64), (BORTEZOMIB:637:10135:60:478:0.95:0.91:0.57:0.70), (AZELASTINE HYDROCHLORIDE:265:10507:12:526:0.95:0.96:0.34:0.50), (ACETAMINOPHENTRAMA DOL:281:10490:33:506:0.95:0.89:0.36:0.51), (MEMANTINE HYDROCHLORIDE:250:10521:3:536:0.95:0.99:0.32:0.48), (ETODOLAC:129:10642:0:539: 0.95:1.00:0.19:0.32), (ROPINIROLE HYDROCHLORIDE:468:10302:54:486:0.95:0.90:0.49:0.63), (LINACLOTIDE:330:10440:17: 523:0.95:0.95:0.39:0.55), (MEGESTROL ACETATE:359:10410:43:498:0.95:0.89:0.42:0.57), (DALTEPARIN SODIUM:383:10385:30:512:0.95:0.93:0.43:0.59), (MOXIFLOXACIN:343:10422:61:484:0.95: 0.85:0.41:0.56), (GANCICLOVIR:289:10476:0:545:0.95:1.00:0.35:0.51), (TAPENTADOL HYDROCHLORIDE:203:10562:5:540:0.95:0.98:0.27:0.43), (GLYBURIDE:553:10211:36:51 0:0.95:0.94:0.52:0.67), (CLOTRIMAZOLE:521:10243:141:405:0.95:0.79:0.56:0.66), (INDO METHACIN:506:10258:104:442:0.95:0.83:0.53:0.65), (BIMATOPROST:355:10409:13:533:0 0.95:0.96:0.40:0.57), (PROGESTERONE:486:10276:56:492:0.95:0.90:0.50:0.64), (TERAZO SINTERAZOSIN HYDROCHLORIDE:237:10525:8:540:0.95:0.97:0.31:0.46), (POTASSIUM CITRATE:155:10607:1:547:0.95:0.99:0.22:0.36), (OXCARBAZEPINE:688:10073:200:349:0. 95:0.77:0.66:0.71), (ASPIRIN DL-LYSINE:119:10642:2:547:0.95:0.98:0.18:0.30), (SILDENAFIL CITRATE:840:9918:0:552:0.95:1.00:0.60:0.75), (BETAMETHASONE VALERATE:163:10595:3:549:0.95:0.98:0.23:0.37), (DEXLANSOPRAZOLE:802:9955:201:3 52:0.95:0.80:0.69:0.74), (HYDROCHLOROTHIAZIDEVALSARTAN:537:10220:59:494:0.9 5:0.90:0.52:0.66), (INDAPAMIDE:474:10283:68:485:0.95:0.87:0.49:0.63), (METAMIZOL E:384:10371:24:531:0.95:0.94:0.42:0.58), (NORTRIPTYLINE:475:10279:65:491:0.95:0.88: 0.49:0.63), (CASPOFUNGIN ACETATE:138:10616:3:553:0.95:0.98:0.20:0.33), (NILOTINIB:431:10321:299:259:0.95:0.5 9:0.62:0.61), (BUMETANIDE:731:10019:154:406:0.95:0.83:0.64:0.72), (ANASTROZOLE: 594:10156:215:345:0.95:0.73:0.63:0.68), (BETAMETHASONE:527:10223:0:560:0.95:1.00: 0.48:0.65), (CEFDINIR:170:10580:6:554:0.95:0.97:0.23:0.38), (SENNA LEAF:141:10609:2:558:0.95:0.99:0.20:0.33), (HYPROMELLOSE 2910 (4000 MPA.S):107:10643:0:560:0.95:1.00:0.16:0.28), (INFLUENZA VIRUS VACCINE:466:10283:54:507:0.95:0.90:0.48:0.62), (OBINUTUZUMAB:124:10625:5:556:0.9 5:0.96:0.18:0.31), (ACETAMINOPHEN\ASPIRIN\CAFFEINE:120:10627:0:563:0.95:1.00:0 0.18:0.30), (ENALAPRIL MALEATE:585:10161:98:466:0.95:0.86:0.56:0.67), (ALFUZOSIN:216:10530:8:556:0.95:0.9 6:0.28:0.43), (PLANTAGO SEED:359:10385:16:550:0.95:0.96:0.39:0.56), (CLOSTRIDIUM BUTYRICUM SPORES STRAIN M-55:253:10491:10:556:0.95:0.96:0.31:0.47), (LOXOPROFEN:145:10599:3:563:0.95:0.98:0.20: 0.34), (LINAGLIPTIN:572:10171:67:500:0.95:0.90:0.53:0.67), (DOXYCYCLINE HYCLATE:239:10504:17:550:0.95:0.93:0.30:0.46), (ROCURONIUM BROMIDE:141:10602:1:566:0.95:0.99:0.20:0.33), (PSEUDOEPHEDRINE HYDROCHLORIDE:198:10543:3:566:0.95:0.99:0.26:0.41), (CLORAZEPATE DIPOTASSIUM:101:10638:0:571:0.95:1.00:0.15:0.26), (OXYBUTYNIN CHLORIDE:455:10283:52:520:0.95:0.90:0.47:0.61), (BROMAZEPAM:451:10287:44:528:0.9 5:0.91:0.46:0.61), (TEPRENONE:175:10563:2:570:0.95:0.99:0.23:0.38), (BRIMONIDINE TARTRATE:273:10463:4:570:0.95:0.99:0.32:0.49), (THEOPHYLLINE ANHYDROUS:306:10428:6:570:0.95:0.98:0.35:0.52), (GEMFIBROZIL:334:10398:8:570:0.9 5:0.98:0.37:0.54), (MUPIROCIN:312:10419:13:566:0.95:0.96:0.36:0.52), (HYDROCHLOR OTHIAZIDELISINOPRIL:634:10096:107:473:0.95:0.86:0.57:0.69), (PANCRELIPASE AMYLASEPANCRELIPASE LIPASEPANCRELIPASE PROTEASE:465:10265:5:575:0.95:0.99:0.45:0.62), (ONABOTULINUMTOXINA:260:10470: 23:557:0.95:0.92:0.32:0.47), (TESTOSTERONE:862:9867:2:579:0.95:1.00:0.60:0.75), (PO MALIDOMIDE:182:10543:9:576:0.95:0.95:0.24:0.38), (MYCOPHENOLATE MOFETILMYCOPHENOLATE MOFETIL HYDROCHLORIDE:719:10005:27:559:0.95:0.96:0.56:0.71), (AMIKACIN:249:10475:21:565: 0.95:0.92:0.31:0.46), (NOREPINEPHRINENOREPINEPHRINE BITARTRATE:209:10515:6:580:0.95:0.97:0.26:0.42), (THIAMINE HYDROCHLORIDE:79:10644:0:587:0.95:1.00:0.12:0.21), (SILODOSIN:266:10454:11:579:0 0.95:0.96:0.31:0.47), (ATROPINE SULFATEDIPHENOXYLATE HYDROCHLORIDE:518:10201:41:550:0.95:0.93:0.49:0.64), (KETOROLAC TROMETHAMINE:162:10556:1:591:0.95:0.99:0.22:0.35), (VITAMIN B:547:10170:117:476:0.95:0.82:0.53:0.65), (REPAGLINIDE:210:10507:6:587:0.95:0.97:0.2 6:0.41), (SODIUM OXYBATE:590:10126:144:450:0.95:0.80:0.57:0.67), (COLCHICINE:652:10061:100:497:0.9 5:0.87:0.57:0.69), (NICOTINE:363:10350:16:581:0.95:0.96:0.38:0.55), (ALBUMIN HUMAN:272:10438:12:588:0.95:0.96:0.32:0.48), (CANDESARTAN:610:10099:140:461:0.9 5:0.81:0.57:0.67), (GLUCOSAMINE:372:10337:7:594:0.95:0.98:0.39:0.55), (SODIUM PICOSULFATE:199:10509:3:599:0.95:0.99:0.25:0.40), (TOBRAMYCIN:291:10415:23:581:0 0.95:0.93:0.33:0.49), (METHOCARBAMOL:484:10221:25:580:0.95:0.95:0.45:0.62), (ISOS ORBIDE DINITRATE:253:10452:7:598:0.95:0.97:0.30:0.46), (CEFUROXIME:308:10395:18:589:0.95: 0.94:0.34:0.50), (MOSAPRIDE CITRATE:197:10506:3:604:0.95:0.99:0.25:0.39), (DAPTOMYCIN:148:10555:5:602:0.95:0.9 7:0.20:0.33), (ALBUMIN (HUMAN):112:10591:1:606:0.95:0.99:0.16:0.27), (TRIMETHOPRIM:134:10567:0:609:0.95: 1.00:0.18:0.31), (GRANISETRON HYDROCHLORIDE:148:10551:2:609:0.95:0.99:0.20:0.33), (LETROZOLE:435:10263:23:58 9:0.95:0.95:0.42:0.59), (ATROPINE:173:10523:7:607:0.95:0.96:0.22:0.36), (CILOSTAZOL: 368:10326:21:595:0.95:0.95:0.38:0.54), (MELPHALAN:329:10365:3:613:0.95:0.99:0.35:0. 52), (RIFAXIMIN:206:10488:8:608:0.95:0.96:0.25:0.40), (RAMELTEON:62:10630:0:618:0.9 5:1.00:0.09:0.17), (CEFTAZIDIME:234:10457:15:604:0.95:0.94:0.28:0.43), (HYDROCHL OROTHIAZIDELOSARTAN POTASSIUM:493:10197:30:590:0.95:0.94:0.46:0.61), (TADALAFIL:562:10126:0:622:0.95 1.00:0.47:0.64), (BACILLUS SUBTILIS\LACTOBACILLUS ACIDOPHILUS\STREPTOCOCCUS FAECAIS:109:10579:0:622:0.95:1.00:0.15:0.26), (DOXAZOSIN MESYLATE:499:10187:63:561:0.94:0.89:0.47:0.62), (DARBEPOETIN ALFA:638:10047:25:600:0.94:0.96:0.52:0.67), (METHYLPHENIDATE:177:10508:5:620:0.9 4:0.97:0.22:0.36), (MOXIFLOXACIN HYDROCHLORIDE:595:10088:132:495:0.94:0.82:0.55:0.65), (CERTOLIZUMAB PEGOL:576:10107:324:303:0.94:0.64:0.66:0.65), (CALCITRIOL:696:9986:73:555:0.94:0.91: 0.56:0.69), (NITROFURANTOIN:600:10076:90:544:0.94:0.87:0.52:0.65), (CRANBERRY:1 73:10502:2:633:0.94:0.99:0.21:0.35), (ENZALUTAMIDE:77:10598:1:634:0.94:0.99:0.11:0.), (ISOSORBIDE MONONITRATE:755:9919:70:566:0.94:0.92:0.57:0.70), (ERYTHROMYCIN:391:10279:45: 595:0.94:0.90:0.40:0.55), (MIRABEGRON:350:10320:10:630:0.94:0.97:0.36:0.52), (GEMC ITABINE HYDROCHLORIDE:190:10480:11:629:0.94:0.95:0.23:0.37), (PALBOCICLIB:212:10457:12: 629:0.94:0.95:0.25:0.40), (CLINDAMYCIN:393:10272:69:576:0.94:0.85:0.41:0.55), (MEC LIZINE HYDROCHLORIDE:351:10314:15:630:0.94:0.96:0.36:0.52), (DICLOFENAC:796:9866:111: 537:0.94:0.88:0.60:0.71), (AMPHETAMINE ASPARTATEAMPHETAMINE SULFATEDEXTROAMPHETAMINE SACCHARATEDEXTROAMPHETAMINE SULFATE:639:10023:96:552:0.94:0.87:0.54:0.66), (ETIZOLAM:159:10503:0:648:0.94:1.00: 0.20:0.33), (DICYCLOMINE HYDROCHLORIDE:586:10075:36:613:0.94:0.94:0.49:0.64), (AMBROXOL HYDROCHLORIDE:148:10513:1:648:0.94:0.99:0.19:0.31), (VANCOMYCIN HYDROCHLORIDE:383:10277:71:579:0.94:0.84:0.40:0.54), (TRIAZOLAM:103:10555:0:65 2:0.94:1.00:0.14:0.24), (SUNITINIB MALATE:306:10348:35:621:0.94:0.90:0.33:0.48), (THIAMINE:205:10448:4:653:0.94:0.98: 0.24:0.38), (TEICOPLANIN:78:10575:0:657:0.94:1.00:0.11:0.19), (IBRUTINIB:663:9989:33 2:326:0.94:0.67:0.67:0.67), (CODEINE:464:10188:35:623:0.94:0.93:0.43:0.59), (BISACOD YL:467:10183:28:632:0.94:0.94:0.42:0.59), (APREPITANT:334:10315:40:621:0.94:0.89:0.3 5:0.50), (FENTANYL CITRATE:338:10310:25:637:0.94:0.93:0.35:0.51), (AMIODARONE HYDROCHLORIDE:648:9999:113:550:0.94:0.85:0.54:0.66), (ZINCZINC CHLORIDE:174:10473:0:663:0.94:1.00:0.21:0.34), (BUPROPION:608:10037:47:618:0.94:0. 93:0.50:0.65), (MANNITOL:98:10545:1:666:0.94:0.99:0.13:0.23), (FLAX SEED:203:10436:1:670:0.94:1.00:0.23:0.38), (BROTIZOLAM:344:10294:17:655:0.94:0.95: 0.34:0.51), (NIACIN:495:10142:31:642:0.94:0.94:0.44:0.60), (CANDESARTAN CILEXETIL:706:9929:110:565:0.94:0.87:0.56:0.68), (CALCIUM CARBONATECHOLECALCIFEROL:788:9845:159:518:0.94:0.83:0.60:0.70), (PEGFILGR ASTIM:533:10100:41:636:0.94:0.93:0.46:0.61), (TRIAMCINOLONE ACETONIDE:796:9835:76:603:0.94:0.91:0.57:0.70), (NEBIVOLOL HYDROCHLORIDE:533:10097:35:645:0.94:0.94:0.45:0.61), (CLOBETASOL:341:10289:7:6 73:0.94:0.98:0.34:0.50), (BENZONATATE:400:10229:20:661:0.94:0.95:0.38:0.54), (APRE MILAST:90:10539:0:681:0.94:1.00:0.12:0.21), (PREDNISOLONEPREDNISOLONE ACETATEPREDNISOLONE SODIUM SUCCINATE:392:10236:70:612:0.94:0.85:0.39:0.53), (GRANISETRON:239:10384:1:686:0.9 4:1.00:0.26:0.41), (METHYLPHENIDATE HYDROCHLORIDE:526:10096:35:653:0.94:0.94:0.45:0.60), (OLMESARTAN MEDOXOMIL:840:9780:0:690:0.94:1.00:0.55:0.71), (VORICONAZOLE:624:9992:5:689:0.9 4:0.99:0.48:0.64), (OXAZEPAM:396:10219:18:677:0.94:0.96:0.37:0.53), (EVOLOCUMAB: 320:10294:595:101:0.94:0.35:0.76:0.48), (CALCIUM GLUCONATE:150:10464:0:696:0.94:1.00:0.18:0.30), (MINOXIDIL:119:10495:0:696:0.94:1 0.00:0.15:0.25), (AMIODARONE:788:9824:104:594:0.94:0.88:0.57:0.69), (BUSPIRONE HYDROCHLORIDE:662:9949:67:632:0.94:0.91:0.51:0.65), (CYCLOBENZAPRINE:721:988 7:63:639:0.94:0.92:0.53:0.67), (INSULIN DETEMIR:715:9893:26:676:0.94:0.96:0.51:0.67), (CARBIDOPALEVODOPA:464:10144:0: 702:0.94:1.00:0.40:0.57), (AMITRIPTYLINE HYDROCHLORIDE:450:10158:23:679:0.94:0.95:0.40:0.56), (GLICLAZIDE:501:10101:32:6 76:0.94:0.94:0.43:0.59), (CLOBETASOL PROPIONATE:72:10530:0:708:0.94:1.00:0.09:0.17), (PRAVASTATIN SODIUM:834:9767:83:626:0.94:0.91:0.57:0.70), (FERROUS SULFATEFERROUS SULFATE, DRIED:775:9826:143:566:0.94:0.84:0.58:0.69), (DOCETAXEL:424:10172:19:695:0.94:0.96: 0.38:0.54), (TRANEXAMIC ACID:245:10350:7:708:0.94:0.97:0.26:0.41), (DULOXETINE:658:9935:54:663:0.94:0.92:0. 50:0.65), (DONEPEZIL HYDROCHLORIDE:196:10396:2:716:0.94:0.99:0.21:0.35), (OFLOXACIN:125:10467:0:718: 0.94:1.00:0.15:0.26), (DOCUSATE:538:10053:36:683:0.94:0.94:0.44:0.60), (LUBIPROSTO NE:201:10388:1:720:0.94:1.00:0.22:0.36), (ALBUTEROL SULFATEIPRATROPIUM BROMIDE:647:9941:48:674:0.94:0.93:0.49:0.64), (BECLOMETHASONE DIPROPIONATE:493:10095:19:703:0.94:0.96:0.41:0.58), (DIETARY SUPPLEMENT:449:10139:13:709:0.94:0.97:0.39:0.55), (IOHEXOL:4:10583:0:723:0.94:1.00: 0.01:0.01), (TRIAMCINOLONE:419:10166:14:711:0.94:0.97:0.37:0.54), (HYDROCORTIS ONE SODIUM SUCCINATE:327:10257:8:718:0.94:0.98:0.31:0.47), (QUETIAPINE:592:9987:68:663:0.94:0 0.90:0.47:0.62), (TIZANIDINE:532:10047:33:698:0.94:0.94:0.43:0.59), (EVEROLIMUS:671: 9907:192:540:0.94:0.78:0.55:0.65), (PRAVASTATIN:791:9784:77:658:0.94:0.91:0.55:0.68), (TELMISARTAN:680:9895:63:672:0.94:0.92:0.50:0.65), (POLYETHYLENE GLYCOLS:695:9877:108:630:0.93:0.87:0.52:0.65), (DILTIAZEM HYDROCHLORIDE:900:9669:104:637:0.93:0.90:0.59:0.71), (MICAFUNGIN SODIUM:99:10469:0:742:0.93:1.00:0.12:0.21), (ARIPIPRAZOLE:751:9816:7:736:0.93:0.99: 0.51:0.67), (SOLIFENACIN SUCCINATE:408:10159:15:728:0.93:0.96:0.36:0.52), (ALUMINUM HYDROXIDEMAGNESIUM CARBONATE:166:10396:1:747:0.93:0.99:0.18:0.31), (LATANOPROST:637:9923:26:724:0. 93:0.96:0.47:0.63), (MAGNESIUM HYDROXIDE:219:10340:1:750:0.93:1.00:0.23:0.37), (INVESTIGATIONAL PRODUCT:355:10199:20:736:0.93:0.95:0.33:0.48), (LEVOCETIRIZINE DIHYDROCHLORIDE:192:10360:1:757:0.93:0.99:0.20:0.34), (METRONIDAZOLEMETR ONIDAZOLE HYDROCHLORIDE:580:9969:66:695:0.93:0.90:0.45:0.60), (DEXAMETHASONE SODIUM PHOSPHATE:227:10322:17:744:0.93:0.93:0.23:0.37), (PALONOSETRON HYDROCHLORIDE:190:10358:9:753:0.93:0.95:0.20:0.33), (PROMETHAZINEPROMETH AZINE HYDROCHLORIDE:700:9843:53:714:0.93:0.93:0.50:0.65), (CLINDAMYCINCLINDAMY CIN PHOSPHATE:637:9906:167:600:0.93:0.79:0.51:0.62), (AZITHROMYCIN DIHYDRATE:485:10058:37:730:0.93:0.93:0.40:0.56), (FLUTICASONEFLUTICASONE PROPIONATE:487:10054:16:753:0.93:0.97:0.39:0.56), (PHYTONADIONE:353:10185:23:74 9:0.93:0.94:0.32:0.48), (DILTIAZEM:932:9605:87:686:0.93:0.91:0.58:0.71), (ERYTHROP OIETIN:613:9924:51:722:0.93:0.92:0.46:0.61), (LACTOBACILLUS ACIDOPHILUS:224:10312:1:773:0.93:1.00:0.22:0.37), (HUMAN RED BLOOD CELL:110:10424:0:776:0.93:1.00:0.12:0.22), (ATEZOLIZUMAB:50:10482:0:778:0.93:1.00: 0.06:0.11), (ALFACALCIDOL:448:10075:58:729:0.93:0.89:0.38:0.53), (BUPRENORPHINE: 97:10425:0:788:0.93:1.00:0.11:0.20), (PERINDOPRIL:487:10034:36:753:0.93:0.93:0.39:0. 55), (ACETAMINOPHENTRAMADOL HYDROCHLORIDE:187:10327:2:794:0.93:0.99:0.19:0.32), (METHADONE HYDROCHLORIDE:539:9971:55:745:0.93:0.91:0.42:0.57), (LIDOCAINE HYDROCHLORIDE:425:10084:48:753:0.93:0.90:0.36:0.51), (GENTAMICIN:302:10207:17: 784:0.93:0.95:0.28:0.43), (ENOXAPARIN:715:9790:137:668:0.93:0.84:0.52:0.64), (PYRID OXINE HYDROCHLORIDE:491:10014:27:778:0.93:0.95:0.39:0.55), (AMPHOTERICIN B:479:10026:15:790:0.93:0.97:0.38:0.54), (FEXOFENADINEFEXOFENADINE HYDROCHLORIDE:151:10354:0:805:0.93:1.00:0.16:0.27), (KETOPROFEN:278:10226:10:7 96:0.93:0.97:0.26:0.41), (OXYGEN:805:9697:108:700:0.93:0.88:0.53:0.67), (OLOPATADINE HYDROCHLORIDE:187:10313:1:809:0.93:0.99:0.19:0.32), (PROPRANOLOLPROPRANOLOL HYDROCHLORIDE:397:10100:16:797:0.93:0.96:0.33:0.49), (DEXCHLORPHENIRAMINE MALEATE:73:10423:0:814:0.93:1.00:0.08:0.15), (HYDRALAZINE HYDROCHLORIDE:614:9881:25:790:0.93:0.96:0.44:0.60), (FEBUXOSTAT:554:9938:28:79 0:0.93:0.95:0.41:0.58), (VERAPAMIL HYDROCHLORIDE:846:9643:92:729:0.93:0.90:0.54:0.67), (GLIPIZIDE:729:9760:57:764:0. 93:0.93:0.49:0.64), (CALCIUMVITAMIN D:708:9780:38:784:0.93:0.95:0.47:0.63), (ACETYLCYSTEINE:244:10243:4:819:0.93:0.98: 0.23:0.37), (CYCLOPHOSPHAMIDE:1485:9000:1:824:0.93:1.00:0.64:0.78), (TORSEMIDE: 849:9634:48:779:0.93:0.95:0.52:0.67), (SENNOSIDES A AND B:168:10314:0:828:0.93:1.00:0.17:0.29), (CIPROFLOXACIN HYDROCHLORIDE:702:9777:46:785:0.93:0.94:0.47:0.63), (LINEZOLID:262:10214:10:824: 0.93:0.96:0.24:0.39), (HYDROCHLOROTHIAZIDETRIAMTERENE:249:10225:1:835:0.93: 1.00:0.23:0.37), (OXYBUTYNIN:346:10127:3:834:0.93:0.99:0.29:0.45), (CEPHALEXIN:7 18:9753:73:766:0.93:0.91:0.48:0.63), (OLANZAPINE:700:9770:75:765:0.93:0.90:0.48:0.63), (TEMAZEPAM:703:9765:25:817:0.93:0.97:0.46:0.63), (MIDAZOLAM HYDROCHLORIDE:234:10233:1:842:0.93:1.00:0.22:0.36), (CYCLOBENZAPRINE HYDROCHLORIDE:687:9779:25:819:0.93:0.96:0.46:0.62), (ESTROGENS, CONJUGATED:491:9975:18:826:0.93:0.96:0.37:0.54), (GEMCITABINEGEMCITABINE HYDROCHLORIDE:229:10237:4:840:0.93:0.98:0.21:0.35), (DIMETHICONE:331:10134:3:8 42:0.93:0.99:0.28:0.44), (CALCIUMCHOLECALCIFEROL:254:10210:2:844:0.93:0.99:0.2 3:0.38), (METAMIZOLE SODIUM:252:10211:0:847:0.93:1.00:0.23:0.37), (IRBESARTAN:665:9796:38:811:0.92:0.95: 0.45:0.61), (ROSUVASTATIN:382:10068:12:848:0.92:0.97:0.31:0.47), (FERROUS SULFATE:771:9676:76:787:0.92:0.91:0.49:0.64), (METHYLCOBALAMIN:167:10280:0:863: 0.92:1.00:0.16:0.28), (ENALAPRIL:989:9452:92:777:0.92:0.91:0.56:0.69), (HYDROMOR PHONE:565:9876:49:820:0.92:0.92:0.41:0.57), (CEFEPIME HYDROCHLORIDE:320:10119:11:860:0.92:0.97:0.27:0.42), (CHLORPHENIRAMINE MALEATE:91:10344:0:875:0.92:1.00:0.09:0.17), (HALOPERIDOL:515:9917:22:856:0.92:0. 96:0.38:0.54), (BUPROPION HYDROCHLORIDE:927:9494:69:820:0.92:0.93:0.53:0.68), (LENALIDOMIDE:817:9603:37: 853:0.92:0.96:0.49:0.65), (NAPROXEN:1007:9412:111:780:0.92:0.90:0.56:0.69), (MIDAZ OLAMMIDAZOLAM HYDROCHLORIDE:284:10134:3:889:0.92:0.99:0.24:0.39), (ACETAMINOPHENHYDROC ODONE:382:10035:9:884:0.92:0.98:0.30:0.46), (FLUTICASONE PROPIONATESALMETEROL XINAFOATE:1212:9202:0:896:0.92:1.00:0.57:0.73), (DIGOXIN:980:9432:53:845:0.92:0.95: 0.54:0.69), (INSULIN HUMAN:775:9636:45:854:0.92:0.95:0.48:0.63), (GUAIFENESIN:448:9962:12:888:0.92:0.97: 0.34:0.50), (EPINEPHRINE:749:9659:102:800:0.92:0.88:0.48:0.62), (RABEPRAZOLE SODIUM:699:9709:51:851:0.92:0.93:0.45:0.61), (CEFTRIAXONE SODIUM:504:9901:48:857:0.92:0.91:0.37:0.53), (LAMOTRIGINE:846:9558:3:903:0.92:1.00: 0.48:0.65), (PROCHLORPERAZINE:207:10195:1:907:0.92:1.00:0.19:0.31), (KETOCONAZ OLE:173:10229:0:908:0.92:1.00:0.16:0.28), (PROMETHAZINE HYDROCHLORIDE:557:9842:18:893:0.92:0.97:0.38:0.55), (BACLOFEN:962:9436:0:912:0. 92:1.00:0.51:0.68), (GLIMEPIRIDE:820:9572:17:901:0.92:0.98:0.48:0.64), (ASPIRIN LYSINE:351:10041:15:903:0.92:0.96:0.28:0.43), (INSULIN NOS:1230:9160:8:912:0.92:0.99:0.57:0.73), (NAPROXEN SODIUM:678:9711:38:883:0.92:0.95:0.43:0.60), (SODIUM BICARBONATE:568:9821:13:908:0.92:0.98:0.38:0.55), (RISPERIDONE:740:9648:2:920:0.9 2:1.00:0.45:0.62), (RIBAVIRIN:194:10193:0:923:0.92:1.00:0.17:0.30), (ZOLEDRONIC ACID:751:9635:5:919:0.92:0.99:0.45:0.62), (PANTOPRAZOLE:907:9477:92:834:0.92:0.91: 0.52:0.66), (ACETAMINOPHENCODEINE PHOSPHATE:793:9589:69:859:0.92:0.92:0.48:0.63), (BISOPROLOL FUMARATE:817:9564:81:848:0.92:0.91:0.49:0.64), (EZETIMIBE:668:9705:29:908:0.92:0.9 6:0.42:0.59), (SENNA LEAFSENNOSIDESSENNOSIDES A AND B:448:9924:7:931:0.92:0.98:0.32:0.49), (PROBIOTICS NOS:627:9744:31:908:0.92:0.95:0.41:0.57), (HYDROMORPHONE HYDROCHLORIDE:647:9723:42:898:0.92:0.94:0.42:0.58), (ZOLPIDEMZOLPIDEM TARTRATE:817:9552:27:914:0.92:0.97:0.47:0.63), (CARBOCYSTEINE:80:10288:0:942:0.9 2:1.00:0.08:0.15), (AMOXICILLINCLAVULANIC ACID:308:10059:8:935:0.92:0.97:0.25:0.40), (MELATONIN:782:9584:41:903:0.92:0.95:0.4 6:0.62), (TOPIRAMATE:949:9414:100:847:0.92:0.90:0.53:0.67), (MAGNESIUM SULFATE:256:10102:2:950:0.92:0.99:0.21:0.35), (INSULIN LISPRO:915:9428:16:951:0.91:0.98:0.49:0.65), (SUCRALFATE:526:9816:11:957:0.91:0.98: 0.35:0.52), (LOXOPROFEN SODIUM:176:10163:0:971:0.91:1.00:0.15:0.27), (TAMSULOSIN:904:9432:59:915:0.91:0.94: 0.50:0.65), (BEVACIZUMAB:452:9882:6:970:0.91:0.99:0.32:0.48), (PROCHLORPERAZINE MALEATE:453:9878:9:970:0.91:0.98:0.32:0.48), (HYDROCODONE:733:9596:19:962:0.91 0.97:0.43:0.60), (MELOXICAM:852:9475:63:920:0.91:0.93:0.48:0.63), (IPRATROPIUM BROMIDE:431:9895:5:979:0.91:0.99:0.31:0.47), (DIPHENHYDRAMINE:789:9531:99:891:0 0.91:0.89:0.47:0.61), (FENOFIBRATE:700:9617:25:968:0.91:0.97:0.42:0.59), (CYANOCOB ALAMINDEXPANTHENOLNIACINAMIDEPYRIDOXINE HYDROCHLORIDE\RIBOFLAVIN 5′-PHOSPHATE SODIUMTHIAMINE HYDROCHLORIDEVITAMIN B COMPLEX:530:9766:7:1007:0.91:0.99:0.34:0.51), (ZOPICLONE:518:9776:21:995:0.91:0.96: 0.34:0.50), (DICLOFENAC SODIUM:885:9408:53:964:0.91:0.94:0.48:0.64), (BIOTIN:405:9886:3:1016:0.91:0.99:0.29: 0.44), (SENNOSIDES:433:9855:10:1012:0.91:0.98:0.30:0.46), (.ALPHA.-TOCOPHEROL:434:9847:4:1025:0.91:0.99:0.30:0.46), (LOPERAMIDE:369:9900:5:1036:0.9 1:0.99:0.26:0.41), (FILGRASTIM:515:9753:11:1031:0.91:0.98:0.33:0.50), (CLONIDINE:74 4:9516:38:1012:0.91:0.95:0.42:0.59), (REBAMIPIDE:200:10059:0:1051:0.91:1.00:0.16:0.28), (OMEGA-3 FATTY ACIDS:412:9846:2:1050:0.91:1.00:0.28:0.44), (DOMPERIDONE:381:9871:5:1053:0.91:0.99: 0.27:0.42), (POTASSIUM:928:9313:45:1024:0.91:0.95:0.48:0.63), (CLARITHROMYCIN:4 07:9834:13:1056:0.91:0.97:0.28:0.43), (NYSTATIN:673:9566:22:1049:0.91:0.97:0.39:0.56), (METRONIDAZOLE:917:9321:107:965:0.91:0.90:0.49:0.63), (HERBALS:339:9899:5:1067: 0.91:0.99:0.24:0.39), (NIFEDIPINE:815:9420:30:1045:0.90:0.96:0.44:0.60), (BUDESONID E:885:9343:70:1012:0.90:0.93:0.47:0.62), (POLYETHYLENE GLYCOL 3350POTASSIUM CHLORIDE\SODIUM BICARBONATE\SODIUM CHLORIDE:138:10089:0:1083:0.90:1.00:0.11:0.20), (IRON:845:9374:51:1040:0.90:0.94:0.4: 0.61), (QUETIAPINE FUMARATE:676:9543:17:1074:0.90:0.98:0.39:0.55), (PRAVASTATINPRAVASTATIN SODIUM:476:9741:7:1086:0.90:0.99:0.30:0.47), (INSULIN ASPART:914:9301:23:1072:0.90:0.98:0.46:0.63), (PROPRANOLOL HYDROCHLORIDE:484:9731:2:1093:0.90:1.00:0.31:0.47), (TAMSULOSIN HYDROCHLORIDE:745:9456:20:1089:0.90:0.97:0.41:0.57), (OMEPRAZOLE MAGNESIUM:1168:9022:29:1091:0.90:0.98:0.52:0.68), (DOXYCYCLINE:607:9583:22:109 8:0.90:0.97:0.36:0.52), (PACLITAXEL:515:9674:6:1115:0.90:0.99:0.32:0.48), (DEXTROS E:177:10011:0:1122:0.90:1.00:0.14:0.24), (AZITHROMYCIN ANHYDROUS:568:9614:18:1110:0.90:0.97:0.34:0.50), (ERGOCALCIFEROL:804:9371:22:1 113:0.90:0.97:0.42:0.59), (MINERALSVITAMINS:355:9819:0:1136:0.90:1.00:0.24:0.38), (UBIDECARENONE:438:9730:0:1142:0.90:1.00:0.28:0.43), (MOMETASONE FUROATE:449:9715:5:1141:0.90:0.99:0.28:0.44), (METHYLPREDNISOLONE SODIUM SUCCINATE:807:9356:131:1016:0.90:0.86:0.44:0.58), (TIOTROPIUM BROMIDE MONOHYDRATE:868:9294:32:1116:0.90:0.96:0.44:0.60), (PROPOFOL:161:9978:0:1171:0. 90:1.00:0.12:0.22), (TRAMADOL HYDROCHLORIDE:710:9426:11:1163:0.90:0.98:0.38:0.55), (ACETAMINOPHENOXYCO DONE HYDROCHLORIDE:698:9424:8:1180:0.89:0.99:0.37:0.54), (FLUTICASONE PROPIONATE:1056:9058:40:1156:0.89:0.96:0.48:0.64), (RANITIDINE:559:9551:5:1195:0.8 9:0.99:0.32:0.48), (NITROGLYCERIN:749:9360:17:1184:0.89:0.98:0.39:0.56), (VALSART AN:977:9115:15:1203:0.89:0.98:0.45:0.62), (HYDROXYZINEHYDROXYZINE HYDROCHLORIDE:242:9850:1:1217:0.89:1.00:0.17:0.28), (LOSARTAN POTASSIUM:640:9442:2:1226:0.89:1.00:0.34:0.51), (RANITIDINE HYDROCHLORIDE:981:9099:14:1216:0.89:0.99:0.45:0.61), (METOPROLOL TARTRATE:832:9242:16:1220:0.89:0.98:0.41:0.57), (HYDROXYZINE HYDROCHLORIDE:226:9846:0:1238:0.89:1.00:0.15:0.27), (FEXOFENADINE HYDROCHLORIDE:424:9647:1:1238:0.89:1.00:0.26:0.41), (AMITRIPTYLINE:789:9280:24: 1217:0.89:0.97:0.39:0.56), (CELECOXIB:921:9143:35:1211:0.89:0.96:0.43:0.60), (WARF ARIN SODIUM:1010:9049:28:1223:0.89:0.97:0.45:0.62), (ESTRADIOL:465:9593:3:1249:0.89:0.9 9:0.27:0.43), (URSODIOL:413:9632:4:1261:0.89:0.99:0.25:0.40), (ESOMEPRAZOLE:709:9 334:26:1241:0.89:0.96:0.36:0.53), (RAMIPRIL:1003:9025:51:1231:0.89:0.95:0.45:0.61), (R ANITIDINERANITIDINE HYDROCHLORIDE:385:9642:0:1283:0.89:1.00:0.23:0.38), (VENLAFAXINE HYDROCHLORIDE:926:9099:16:1269:0.89:0.98:0.42:0.59), (DOCUSATE SODIUM:914:9107:34:1255:0.89:0.96:0.42:0.59), (FISH OIL:1008:9010:17:1275:0.89:0.98:0.44:0.61), (TRAZODONE HYDROCHLORIDE:965:9021:17:1307:0.88:0.98:0.42:0.59), (AMOXICILLIN:775:9209:25: 1301:0.88:0.97:0.37:0.54), (WARFARIN:1078:8901:34:1297:0.88:0.97:0.45:0.62), (LOPER AMIDE HYDROCHLORIDE:324:9651:0:1335:0.88:1.00:0.20:0.33), (MEROPENEM:456:9514:9:133 1:0.88:0.98:0.26:0.40), (MAGNESIUM:890:9074:20:1326:0.88:0.98:0.40:0.57), (DULOXE TINE HYDROCHLORIDE:961:8994:6:1349:0.88:0.99:0.42:0.59), (HEPARIN SODIUM:930:9023:45:1312:0.88:0.95:0.41:0.58), (CALCIUM CARBONATE:730:9215:4:1361:0.88:0.99:0.35:0.52), (MORPHINE:825:9116:14:1355:0.88: 0.98:0.38:0.55), (AMOXICILLINCLAVULANATE POTASSIUM:527:9414:6:1363:0.88:0.99:0.28:0.43), (BUDESONIDEFORMOTEROL FUMARATE DIHYDRATE:641:9293:3:1373:0.88:1.00:0.32:0.48), (BISOPROLOL:688:9245:11:1366:0.88: 0.98:0.33:0.50), (CALCIUM:1290:8617:18:1385:0.88:0.99:0.48:0.65), (POLYETHYLENE GLYCOL 3350:698:9207:10:1395:0.88:0.99:0.33:0.50), (LIDOCAINE:748:9156:20:1386:0.88:0.97:0.3 5:0.52), (METOCLOPRAMIDE:308:9580:1:1421:0.87:1.00:0.18:0.30), (CITALOPRAM HYDROBROMIDE:1000:8887:23:1400:0.87:0.98:0.42:0.58), (LOSARTAN:985:8878:3:1444: 0.87:1.00:0.41:0.58), (ESCITALOPRAM OXALATE:996:8863:18:1433:0.87:0.98:0.41:0.58), (VALACYCLOVIR HYDROCHLORIDE:599:9260:10:1441:0.87:0.98:0.29:0.45), (MONTELUKAST SODIUM:940:8875:10:1485:0.87:0.99:0.39:0.56), (METOPROLOL SUCCINATE:683:9127:3:1497:0.87:1.00:0.31:0.48), (LACTULOSE:347:9462:1:1500:0.87:1 0.00:0.19:0.32), (FLUCONAZOLE:770:9023:12:1505:0.87:0.98:0.34:0.50), (INSULIN GLARGINE:946:8845:4:1515:0.87:1.00:0.38:0.55), (LORATADINE:822:8955:11:1522:0.86: 0.99:0.35:0.52), (ALBUTEROL SULFATE:1087:8689:7:1527:0.86:0.99:0.42:0.59), (ENOXAPARIN SODIUM:722:9050:13:1525:0.86:0.98:0.32:0.48), (MORPHINE SULFATE:406:9342:0:1562:0.86:1.00:0.21:0.34), (ROSUVASTATIN CALCIUM:943:8804:5:1558:0.86:0.99:0.38:0.55), (HYDROCORTISONE:774:8962:0:1574:0 0.86:1.00:0.33:0.50), (MIRTAZAPINE:357:9363:0:1590:0.86:1.00:0.18:0.31), (METOCLOP RAMIDE HYDROCHLORIDE:415:9303:0:1592:0.86:1.00:0.21:0.34), (VANCOMYCIN:633:9080:6:15 91:0.86:0.99:0.28:0.44), (SERTRALINE HYDROCHLORIDE:1061:8651:8:1590:0.86:0.99:0.40:0.57), (ATENOLOL:947:8746:8:1609: 0.86:0.99:0.37:0.54), (SPIRONOLACTONE:1052:8610:18:1630:0.85:0.98:0.39:0.56), (CIP ROFLOXACIN:637:9011:9:1653:0.85:0.99:0.28:0.43), (PIPERACILLIN SODIUMTAZOBACTAM SODIUM:335:9306:0:1669:0.85:1.00:0.17:0.29), (CETIRIZINE HYDROCHLORIDE:903:8713:7:1687:0.85:0.99:0.35:0.52), (OXYCODONE HYDROCHLORIDE:598:9015:6:1691:0.85:0.99:0.26:0.41), (ZOLPIDEM TARTRATE:771:8828:4:1707:0.85:0.99:0.31:0.47), (CLOPIDOGREL BISULFATE:1028:8564:4:1714:0.85:1.00:0.37:0.54), (CARVEDILOL:900:8687:5:1718:0.85: 0.99:0.34:0.51), (FOLIC ACID:1508:8070:11:1721:0.85:0.99:0.47:0.64), (ACETAMINOPHENHYDROCODONE BITARTRATE:821:8742:2:1745:0.85:1.00:0.32:0.48), (TRAMADOL:1014:8538:7:1751:0.84: 0.99:0.37:0.54), (ASCORBIC ACID:710:8838:3:1759:0.84:1.00:0.29:0.45), (ATORVASTATIN CALCIUM:1089:8433:9:1779:0.84:0.99:0.38:0.55), (OXYCODONE:733:8780:5:1792:0.84:0 0.99:0.29:0.45), (DIPHENHYDRAMINE HYDROCHLORIDE:585:8923:3:1799:0.84:0.99:0.25:0.39), (DIAZEPAM:685:8746:1:1878:0 0.83:1.00:0.27:0.42), (HYDROCHLOROTHIAZIDE:827:8597:0:1886:0.83:1.00:0.30:0.47), (CLONAZEPAM:1003:8404:4:1899:0.83:1.00:0.35:0.51), (MAGNESIUM OXIDE:363:9039:0:1908:0.83:1.00:0.16:0.28), (ALBUTEROL:913:8481:6:1910:0.83:0.99:0. 32:0.49), (ACYCLOVIR:565:8827:3:1915:0.83:0.99:0.23:0.37), (SODIUM CHLORIDE:726:8646:9:1929:0.83:0.99:0.27:0.43), (IBUPROFEN:1193:8156:10:1951:0.83:0 0.99:0.38:0.55), (FAMOTIDINE:569:8780:0:1961:0.83:1.00:0.22:0.37), (LANSOPRAZOLE:9 72:8374:13:1951:0.83:0.99:0.33:0.50), (LEVOFLOXACIN:743:8596:3:1968:0.83:1.00:0.27: 0.43), (UNSPECIFIED INGREDIENT:1234:8092:23:1961:0.82:0.98:0.39:0.55), (ALLOPURINOL:889:8437:5:1979: 0.82:0.99:0.31:0.47), (ESOMEPRAZOLE MAGNESIUM:1122:8164:7:2017:0.82:0.99:0.36:0.53), (METOPROLOL:863:8402:1:2044:0. 82:1.00:0.30:0.46), (CYANOCOBALAMIN:783:8452:1:2074:0.82:1.00:0.27:0.43), (FENTA NYL:620:8580:0:2110:0.81:1.00:0.23:0.37), (POTASSIUM CHLORIDE:884:8277:0:2149:0.81:1.00:0.29:0.45), (ALPRAZOLAM:929:8219:0:2162:0.81: 1.00:0.30:0.46), (SIMVASTATIN:1031:8108:4:2167:0.81:1.00:0.32:0.49), (ATORVASTATI N:829:8286:1:2194:0.81:1.00:0.27:0.43), (METFORMIN HYDROCHLORIDE:1267:7732:2:2309:0.80:1.00:0.35:0.52), (VITAMINS:1123:7859:1:2327: 0.79:1.00:0.33:0.49), (PREGABALIN:854:8089:1:2366:0.79:1.00:0.27:0.42), (LISINOPRIL: 963:7974:0:2373:0.79:1.00:0.29:0.45), (LEVOTHYROXINE SODIUM:1140:7718:1:2451:0.78:1.00:0.32:0.48), (LEVOTHYROXINE:1138:7705:1:2466:0. 78:1.00:0.32:0.48), (SULFAMETHOXAZOLETRIMETHOPRIM:663:8171:0:2476:0.78:1.0 0:0.21:0.35), (DEXAMETHASONE:885:7910:1:2514:0.78:1.00:0.26:0.41), (PANTOPRAZOLE SODIUM:1141:7618:0:2551:0.77:1.00:0.31:0.47), (LORAZEPAM:607:8137:0:2566:0.77:1.0 0:0.19:0.32), (GABAPENTIN:878:7793:1:2638:0.77:1.00:0.25:0.40), (PREDNISONE:1428: 7208:1:2673:0.76:1.00:0.35:0.52), (AMLODIPINE BESYLATE:1046:7409:0:2855:0.75:1.00:0.27:0.42), (OMEPRAZOLE:1072:7243:0:2995:0.7 4:1.00:0.26:0.42), (CHOLECALCIFEROL:1125:7111:0:3074:0.73:1.00:0.27:0.42), (FUROS EMIDE:843:7042:0:3425:0.70:1.00:0.20:0.33), (ACETAMINOPHEN:955:6561:0:3794:0.66: 1.00:0.20:0.33), (ASPIRIN:1146:6298:0:3866:0.66:1.00:0.23:0.37)

### <Data List 2>

The TP, TN, FP, FN, Accuracy score, Precision score, Recall score, F_measure score are shown for each indication in the following order (Indication:TP:TN:FP:FN:Accuracy:Precision:Recall:F_measure). “#NUM!” means that “0” is included in the values and calculation cannot be therefore made.

(Lupus.vulgaris:6:4879:0:0:1.00:1.00:1.00:1.00), (Superficial.spreading.melanoma.stage.III:4:48 81:0:0:1.00:1.00:1.00:1.00), (Diaphragm.neoplasm:3:4882:0:0:01.00:1.00:1.00:1.00), (Actinic.elast osis:2:4883:0:0:1.00:1.00:1.00:1.00), (Arterial.graft:2:4883:0:0:1.00:1.00:1.00:1.00), (Benign.uter ine.neoplasm:2:4883:0:0:1.00:1.00:1.00:1.00), (Body.height:2:4883:0:0:1.00:1.00:1.00:1.00), (Car dio-respiratory.distress:2:4883:0:0:1.00:1.00:1.00:1.00), (Choroidal.haemangioma:2:4883:0:0:1.00:1. 00:1.00:1.00), (Corrected.transposition.of.great.vessels:2:4883:0:0:1.00:1.00:1.00:1.00), (Detach ment.of.macular.retinal.pigment.epithelium:2:4883:0:0:1.00:1.00:1.00:1.00), (Dyschondrosteosis: 2:4883:0:0:1.00:1.00:1.00:1.00), (Encephalocele:2:4883:0:0:1.00:1.00:1.00:1.00), (Epiphyses.pre mature.fusion:2:4883:0:0:1.00:1.00:1.00:1.00), (Evacuation.of.retained.products.of.conception:2: 4883:0:0:1.00:1.00:1.00:1.00), (Liver.iron.concentration.increased:2:4883:0:0:1.00:1.00:1.00:1.0 0), (Mediastinal.fibrosis:2:4883:0:0:1.00:1.00:1.00:1.00), (Mismatch.repair.cancer.syndrome:2:48 83:0:0:1.00:1.00:1.00:1.00), (Polyglandular.disorder:2:4883:0:0:1.00:1.00:1.00:1.00), (Retinal.me lanocytoma:2:4883:0:0:1.00:1.00:1.00:1.00), (Testicular.choriocarcinoma.stage.III:2:4883:0:0:1.0 0:1.00:1.00:1.00), (Uterine.cervix.stenosis:2:4883:0:0:1.00:1.00:1.00:1.00), (Venous.pressure.incr eased:2:4883:0:0:1.00:1.00:1.00:1.00), (Von.Willebrand's.factor.inhibition:2:4883:0:0:1.00:1.00: 1.00:1.00), (Abnormal.withdrawal.bleeding:1:4884:0:0:1.00:1.00:1.00:1.00), (ABO.haemolytic.di sease.of.newborn:1:4884:0:0:1.00:1.00:1.00:1.00), (Aborted.pregnancy:1:4884:0:0:1.00:1.00:1.00: 1.00), (Administration.related.reaction:1:4884:0:0:1.00:1.00:1.00:1.00), (Alpha.tumour.necrosis.f actor.increased:1:4884:0:0:1.00:1.00:1.00:1.00), (Amegakaryocytic.thrombocytopenia:1:4884:0:0: 1.00:1.00:1.00:1.00), (Amniotic.membrane.graft:1:4884:0:0:1.00:1.00:1.00:1.00), (Anastomotic.u lcer.haemorrhage:1:4884:0:0:1.00:1.00:1.00:1.00), (Anembryonic.gestation:1:4884:0:0:1.00:1.00: 1.00:1.00), (Ankle.deformity:1:4884:0:0:1.00:1.00:1.00:1.00), (Anovulvar.fistula:1:4884:0:0:1.00: 1.00:1.00:1.00), (Anterior.spinal.artery.syndrome:1:4884:0:0:1.00:1.00:1.00:1.00), (Anti-myelin-associated.glycoprotein.antibodies.positive:1:4884:0:0:1.00:1.00:1.00:1.00), (Apocrine.miliaria:1: 4884:0:0:1.00:1.00:1.00:1.00), (Atlantoaxial.instability:1:4884:0:0:1.00:1.00:1.00:1.00), (Autoim mune.demyelinating.disease:1:4884:0:0:1.00:1.00:1.00:1.00), (Bladder.transitional.cell.carcinom a.stage.II:1:4884:0:0:1.00:1.00:1.00:1.00), (Bleeding.anovulatory:1:4884:0:0:1.00:1.00:1.00:1.0 0), (Blood.glucagon:1:4884:0:0:1.00:1.00:1.00:1.00), (Bone.prosthesis.insertion:1:4884:0:0:1.00:1 0.00:1.00:1.00), (Bronchial.anastomosis:1:4884:0:0:1.00:1.00:1.00:1.00), (Bronchopleural.fistula:1: 4884:0:0:1.00:1.00:1.00:1.00), (Bulbospinal.muscular.atrophy.congenital:1:4884:0:0:1.00:1.00:1. 00:1.00), (Cervical.dilatation:1:4884:0:0:1.00:1.00:1.00:1.00), (Cervix.carcinoma.stage.0:1:4884: 0:0:1.00:1.00:1.00:1.00), (Cervix.enlargement:1:4884:0:0:1.00:1.00:1.00:1.00), (Congenital.foot. malformation:1:4884:0:0:1.00:1.00:1.00:1.00), (Conjunctival.primary.acquired.melanosis:1:4884: 0:0:1.00:1.00:1.00:1.00), (Contraceptive.rhythm.method:1:4884:0:0:1.00:1.00:1.00:1.00), (Costell o.syndrome:1:4884:0:0:1.00:1.00:1.00:1.00), (CSF.white.blood.cell.count:1:4884:0:0:1.00:1.00:1. 00:1.00), (Device.breakage:1:4884:0:0:1.00:1.00:1.00:1.00), (Device.expulsion:1:4884:0:0:1.00:1. 00:1.00:1.00), (Diabetic.blindness:1:4884:0:0:1.00:1.00:1.00:1.00), (Diffuse.alveolar.damage:1:48 84:0:0:1.00:1.00:1.00:1.00), (DNA.mismatch.repair.protein.gene.mutation:1:4884:0:0:1.00:1.00:1 0.00:1.00), (Drowning:1:4884:0:0:1.00:1.00:1.00:1.00), (Drug.effect.delayed:1:4884:0:0:1.00:1.00: 1.00:1.00), (Ear.malformation:1:4884:0:0:1.00:1.00:1.00:1.00), (Echovirus.test.positive:1:4884:0: 0:1.00:1.00:1.00:1.00), (Endocrine.gland.operation:1:4884:0:0:1.00:1.00:1.00:1.00), (Endometrios is.ablation:1:4884:0:0:1.00:1.00:1.00:1.00), (Enterovirus.test.positive:1:4884:0:0:1.00:1.00:1.00:1 0.00), (Erythrokeratodermia.variabilis:1:4884:0:0:1.00:1.00:1.00:1.00), (Extensor.plantar.response: 1:4884:0:0:1.00:1.00:1.00:1.00), (Factor.Xa.activity.normal:1:4884:0:0:1.00:1.00:1.00:1.00), (Fail ed.in.vitro.fertilisation:1:4884:0:0:1.00:1.00:1.00:1.00), (Fluorosis:1:4884:0:0:1.00:1.00:1.00:1.0 0), (Foetal.anticonvulsant.syndrome:1:4884:0:0:1.00:1.00:1.00:1.00), (Fracture.delayed.union:1:4 884:0:0:1.00:1.00:1.00:1.00), (Fractured.ischium:1:4884:0:0:1.00:1.00:1.00:1.00), (Freeman-Sheldon.syndrome:1:4884:0:0:01.00:1.00:1.00:1.00), (Gastrointestinal.stenosis:1:4884:0:0:1.00:1. 00:1.00:1.00), (Haemoglobin.C.disease:1:4884:0:0:1.00:1.00:1.00:1.00), (Haemorrhage.foetal:1:4 884:0:0:1.00:1.00:1.00:1.00), (Haemosiderinuria:1:4884:0:0:1.00:1.00:1.00:1.00), (Hepatitis.B.e.a ntigen.negative:1:4884:0:0:1.00:1.00:1.00:1.00), (HLA.marker.study:1:4884:0:0:1.00:1.00:1.00:1 0.00), (Human.chorionic.gonadotropin.decreased:1:4884:0:0:1.00:1.00:1.00:1.00), (Imprisonment:1: 4884:0:0:1.00:1.00:1.00:1.00), (Incision.site.rash:1:4884:0:0:1.00:1.00:1.00:1.00), (Instillation.sit e.pain:1:4884:0:0:1.00:1.00:1.00:1.00), (Instillation.site.pruritus:1:4884:0:0:1.00:1.00:1.00:1.00), (Intercepted.drug.administration.error:1:4884:0:0:1.00:1.00:1.00:1.00), (Iridectomy:1:4884:0:0:1. 00:1.00:1.00:1.00), (Iridoschisis:1:4884:0:0:1.00:1.00:1.00:1.00), (Kearns-Sayre.syndrome:1:4884:0:0:1.00:1.00:1.00:1.00), (Kleihauer-Betke.test:1:4884:0:0:1.00:1.00:1.00:1.00), (Kleihauer-Betke.test.positive:1:4884:0:0:1.00:1.00:1.00:1.00), (Laurence-Moon-Bardet-Biedl.syndrome:1:4884:0:0:1.00:1.00:1.00:1.00), (Lip.exfoliation:1:4884:0:0:1.00:1.00:1.00:1.00), (Lordosis:1:4884:0:0:1.00:1.00:1.00:1.00), (Lymphangiopathy:1:4884:0:0:1.00:1.00:1.00:1.00), (Lymphocyte.adoptive.therapy:1:4884:0:0:1.00:1.00:1.00:1.00), (Macular.dystrophy.congenital:1: 4884:0:0:1.00:1.00:1.00:1.00), (Macular.scar:1:4884:0:0:1.00:1.00:1.00:1.00), (Mast.cell.degranu lation.test:1:4884:0:0:1.00:1.00:1.00:1.00), (Medical.anabolic.therapy:1:4884:0:0:1.00:1.00:1.00: 1.00), (Meningocele.repair:1:4884:0:0:1.00:1.00:1.00:1.00), (Mesenteric.vascular.occlusion:1:488 4:0:0:1.00:1.00:1.00:1.00), (Metaphyseal.dysplasia:1:4884:0:0:1.00:1.00:1.00:1.00), (Micromastia: 1:4884:0:0:1.00:1.00:1.00:1.00), (Missed.labour:1:4884:0:0:1.00:1.00:1.00:1.00), (Multiparous:1: 4884:0:0:1.00:1.00:1.00:1.00), (Multiple.use.of.single-use.product:1:4884:0:0:1.00:1.00:1.00:1.00), (Neonatal.haemochromatosis:1:4884:0:0:1.00:1.00: 1.00:1.00), (Netherton's.syndrome:1:4884:0:0:1.00:1.00:1.00:1.00), (Neuropathic.muscular.atroph y:1:4884:0:0:1.00:1.00:1.00:1.00), (Neutropenia.neonatal:1:4884:0:0:1.00:1.00:1.00:1.00), (Occup ational.problem.environmental:1:4884:0:0:1.00:1.00:1.00:1.00), (Oesophagobronchial.fistula:1:4 884:0:0:1.00:1.00:1.00:1.00), (Optic.nerve.hypoplasia:1:4884:0:0:1.00:1.00:1.00:1.00), (Osteomy elitis.drainage:1:4884:0:0:1.00:1.00:1.00:1.00), (Ovarian.theca.cell.tumour:1:4884:0:0:1.00:1.00: 1.00:1.00), (Palmar.fasciitis:1:4884:0:0:1.00:1.00:1.00:1.00), (Parechovirus.infection:1:4884:0:0:1 0.00:1.00:1.00:1.00), (Pelvic.haemorrhage:1:4884:0:0:1.00:1.00:1.00:1.00), (Physical.examination. abnormal:1:4884:0:0:1.00:1.00:1.00:1.00), (Pierre.Robin.syndrome:1:4884:0:0:1.00:1.00:1.00:1.0 0), (Portal.vein.cavernous.transformation:1:4884:0:0:1.00:1.00:1.00:1.00), (Pregnancy.with.advan ced.maternal.age:1:4884:0:0:1.00:1.00:1.00:1.00), (Prolonged.pregnancy:1:4884:0:0:1.00:1.00:1. 00:1.00), (Prothrombin.time.abnormal:1:4884:0:0:1.00:1.00:1.00:1.00), (Psoriasis.area.severity.in dex:1:4884:0:0:1.00:1.00:1.00:1.00), (Renal.atrophy:1:4884:0:0:1.00:1.00:1.00:1.00), (Retinal.inj ury:1:4884:0:0:1.00:1.00:1.00:1.00), (Reye's.syndrome:1:4884:0:0:1.00:1.00:1.00:1.00), (Rhesus. haemolytic.disease.of.newborn:1:4884:0:0:1.00:1.00:1.00:1.00), (Sacroiliac.fracture:1:4884:0:0:1 0.00:1.00:1.00:1.00), (Scapula.fracture:1:4884:0:0:1.00:1.00:1.00:1.00), (Sclerosing.encapsulating. peritonitis:1:4884:0:0:1.00:1.00:1.00:1.00), (Secondary.transmission:1:4884:0:0:1.00:1.00:1.00:1. 00), (Shoshin.beriberi:1:4884:0:0:1.00:1.00:1.00:1.00), (Shrinking.lung.syndrome:1:4884:0:0:1.00:1.00:1.00:1.00), (Silicon.granuloma:1:4884:0:0:1.00:1.00:1.00:1.00), (Spinal.cord.injury.lumbar: 1:4884:0:0:1.00:1.00:1.00:1.00), (Subchorionic.haemorrhage:1:4884:0:0:1.00:1.00:1.00:1.00), (Sy ringe.issue:1:4884:0:0:1.00:1.00:1.00:1.00), (Tilted.disc.syndrome:1:4884:0:0:1.00:1.00:1.00:1.0 0), (Twin.reversed.arterial.perfusion.sequence.malformation:1:4884:0:0:1.00:1.00:1.00:1.00), (Un intended.pregnancy:1:4884:0:0:1.00:1.00:1.00:1.00), (Unwanted.pregnancy:1:4884:0:0:1.00:1.00: 1.00:1.00), (Uraemic.encephalopathy:1:4884:0:0:1.00:1.00:1.00:1.00), (Uterine.carcinoma.in.situ: 1:4884:0:0:1.00:1.00:1.00:1.00), (Uterine.cervical.exfoliation:1:4884:0:0:1.00:1.00:1.00:1.00), (U terine.dilation.and.evacuation:1:4884:0:0:1.00:1.00:1.00:1.00), (Uterine.mass:1:4884:0:0:1.00:1.0 0:1.00:1.00), (Uveitic.glaucoma:1:4884:0:0:1.00:1.00:1.00:1.00), (Vaccination.failure:1:4884:0:0: 1.00:1.00:1.00:1.00), (Vascular.dissection:1:4884:0:0:1.00:1.00:1.00:1.00), (Venous.angioma.of.b rain:1:4884:0:0:1.00:1.00:1.00:1.00), (Vitreous.loss:1:4884:0:0:1.00:1.00:1.00:1.00), (11-beta-hydroxylase.deficiency:0:4884:0:1:1.00:#NUM!:0.00:#NUM!), (5-hydroxyindolacetic.acid.in.urine.increased:0:4884:0:1:1.00:#NUM!:0.00:#NUM!), (Abdominal.e xploration:0:4884:0:1:1.00:#NUM!:0.00:#NUM!), (Abdominal.X-ray:0:4884:0:1:1.00:#NUM!:0.00:#NUM!), (Abnormal.palmar/plantar.creases:0:4884:0:1:1.00:#NUM!:0.00:#NUM!), (Abortion.induced.complicated:0:4884:0:1:1.00:#NUM!:0.00:#NUM!), (Ab scess.of.external.auditory.meatus:0:4884:0:1:1.00:#NUM!:0.00:#NUM!), (Accident.at.home:0:48 84:0:1:1.00:#NUM!:0.00:#NUM!), (Accidental.exposure.to.product.packaging:0:4884:0:1:1.00:#NUM!:0.00:#NUM!), (Acquired.afibrinogenaemia:0:4884:0:1:1.00:#NUM!:0.00:#NUM!), (Acqui red.cystic.kidney.disease:0:4884:0:1:1.00:#NUM!:0.00:#NUM!), (Acral.lentiginous.melanoma.st age.III:0:4884:0:1:1.00:#NUM!:0.00:#NUM!), (Actinic.prurigo:0:4884:0:1:1.00:#NUM!:0.00:#N UM!), (Activated.partial.thromboplastin.time.abnormal:0:4884:0:1:1.00:#NUM!:0.00:#NUM!), (Activated.partial.thromboplastin.time.shortened:0:4884:0:1:1.00:#NUM!:0.00:#NUM!), (Adenoi dectomy:0:4884:0:1:1.00:#NUM!:0.00:#NUM!), (Administration.site.erythema:0:4884:0:1:1.00: #NUM!:0.00:#NUM!), (Administration.site.joint.pain:0:4884:0:1:1.00:#NUM!:0.00:#NUM!), (A dministration. site. rash:0:4884:0:1:1.00:#NUM!:0.00:#NUM!), (Adrenal.androgen.excess:0:4884: 0:1:1.00:#NUM!:0.00:#NUM!), (AIDS.dementia.complex:0:4884:0:1:1.00:#NUM!:0.00:#NUM!), (Airway.complication.of.anaesthesia:0:4884:0:1:1.00:#NUM!:0.00:#NUM!), (Alcohol.interactio n:0:4884:0:1:1.00:#NUM!:0.00:#NUM!), (Alien.limb.syndrome:0:4884:0:1:1.00:#NUM!:0.00:#NUM!), (Allergy.test.positive:0:4884:0:1:1.00:#NUM!:0.00:#NUM!), (Allergy.to.vaccine:0:4884: 0:1:1.00:#NUM!:0.00:#NUM!), (Allergy.to.venom:0:4884:0:1:1.00:#NUM!:0.00:#NUM!), (Alles cheriosis:0:4884:0:1:1.00:#NUM!:0.00:#NUM!), (Alpers'.disease:0:4884:0:1:1.00:#NUM!:0.00:#NUM!), (Alpha-1.acid.glycoprotein.abnormal:0:4884:0:1:1.00:#NUM!:0.00:#NUM!), (Alpha-1. anti-trypsin.increased:0:4884:0:1:1.00:#NUM!:0.00:#NUM!), (Alpha.1.foetoprotein.abnormal:0:4884: 0:1:1.00:#NUM!:0.00:#NUM!), (Alpha.1.globulin.decreased:0:4884:0:1:1.00:#NUM!:0.00:#NUM!), (Aluminium.overload:0:4884:0:1:1.00:#NUM!:0.00:#NUM!), (Amoeba.test.positive:0:4884: 0:1:1.00:#NUM!:0.00:#NUM!), (Amputee:0:4884:0:1:1.00:#NUM!:0.00:#NUM!), (Amylase.decr eased:0:4884:0:1:1.00:#NUM!:0.00:#NUM!), (Amyloidoma:0:4884:0:1:1.00:#NUM!:0.00:#NUM!), (Anal.polyp:0:4884:0:1:1.00:#NUM!:0.00:#NUM!), (Anaphylactoid.shock:0:4884:0:1:1.00:#NUM!:0.00:#NUM!), (Anastomotic.stenosis:0:4884:0:1:1.00:#NUM!:0.00:#NUM!), (Angiocentri c. lymphoma.recurrent:0:4884:0:1:1.00:#NUM!:0.00:#NUM!), (Anorexia.and.bulimia.syndrome: 0:4884:0:1:1.00:#NUM!:0.00:#NUM!), (Anoxia:0:4884:0:1:1.00:#NUM!:0.00:#NUM!), (Anti.fac tor.VIII.antibody.positive:0:4884:0:1:1.00:#NUM!:0.00:#NUM!), (Antibiotic.level.above.therape utic:0:4884:0:1:1.00:#NUM!:0.00:#NUM!), (Antibody.test:0:4884:0:1:1.00:#NUM!:0.00:#NUM!), (Antidiuretic.hormone.abnormality:0:4884:0:1:1.00:#NUM!:0.00:#NUM!), (Antipsychotic.drug.level.below.therapeutic:0:4884:0:1:1.00:#NUM!:0.00:#NUM!), (Aorta.hypoplasia:0:4884:0:1:1. 00:#NUM!:0.00:#NUM!), (Aphakia:0:4884:0:1:1.00:#NUM!:0.00:#NUM!), (Apicectomy:0:4884: 0:1:1.00:#NUM!:0.00:#NUM!), (Apoptosis:0:4884:0:1:1.00:#NUM!:0.00:#NUM!), (Appendicolit h:0:4884:0:1:1.00:#NUM!:0.00:#NUM!), (Application.site.cellulitis:0:4884:0:1:1.00:#NUM!:0.0 0:#NUM!), (Application.site.dermatitis:0:4884:0:1:1.00:#NUM!:0.00:#NUM!), (Application.site. eczema:0:4884:0:1:1.00:#NUM!:0.00:#NUM!), (Application.site.hypersensitivity:0:4884:0:1:1.0 0:#NUM!:0.00:#NUM!), (Application.site.papules:0:4884:0:1:1.00:#NUM!:0.00:#NUM!), (Appli cation.site.urticaria:0:4884:0:1:1.00:#NUM!:0.00:#NUM!), (Arterial.bypass.occlusion:0:4884:0:1: 1.00:#NUM!:0.00:#NUM!), (Arteriogram.renal:0:4884:0:1:1.00:#NUM!:0.00:#NUM!), (Arthritis.viral:0:4884:0:1:1.00:#NUM!:0.00:#NUM!), (Arthrofibrosis:0:4884:0:1:1.00:#NUM!:0.00:#NU M!), (Arthropod-borne.disease:0:4884:0:1:1.00:#NUM!:0.00:#NUM!), (Arthropod.infestation:0:4884:0:1:1.00:#N UM!:0.00:#NUM!), (Atypical.attention.deficit.syndrome:0:4884:0:1:1.00:#NUM!:0.00:#NUM!), (Azoospermia:0:4884:0:1:1.00:#NUM!:0.00:#NUM!), (B-cell.small.lymphocytic.lymphoma.stage.II:0:4884:0:1:1.00:#NUM!:0.00:#NUM!), (B-lymphocyte.count.increased:0:4884:0:1:1.00:#NUM!:0.00:#NUM!), (Bacterial.food.poisoning:0: 4884:0:1:1.00:#NUM!:0.00:#NUM!), (Bacterial.toxaemia:0:4884:0:1:1.00:#NUM!:0.00:#NUM!), (Balanoposthitis.infective:0:4884:0:1:1.00:#NUM!:0.00:#NUM!), (Basosquamous.carcinoma. of skin:0:4884:0:1:1.00:#NUM!:0.00:#NUM!), (Becker's.muscular.dystrophy:0:4884:0:1:1.00:#NU M!:0.00:#NUM!), (Benign.cartilage.neoplasm:0:4884:0:1:1.00:#NUM!:0.00:#NUM!), (Benign.ea r.neoplasm:0:4884:0:1:1.00:#NUM!:0.00:#NUM!), (Benign.ethnic.neutropenia:0:4884:0:1:1.00:#NUM!:0.00:#NUM!), (Benign.laryngeal.neoplasm:0:4884:0:1:1.00:#NUM!:0.00:#NUM!), (Benig n.neoplasm.of.islets.of.Langerhans:0:4884:0:1:1.00:#NUM!:0.00:#NUM!), (Benign.neoplasm.of retina:0:4884:0:1:1.00:#NUM!:0.00:#NUM!), (Benign.neoplasm.of.scrotum:0:4884:0:1:1.00:#N UM!:0.00:#NUM!), (Benign.spleen.tumour:0:4884:0:1:1.00:#NUM!:0.00:#NUM!), (Bezold-Jarisch.reflex:0:4884:0:1:1.00:#NUM!:0.00:#NUM!), (Bile.duct.cancer.stage.III:0:4884:0:1:1.00: #NUM!:0.00:#NUM!), (Bile.duct.squamous.cell.carcinoma:0:4884:0:1:1.00:#NUM!:0.00:#NUM!), (Biliary. cyst:0:4884:0:1:1.00:#NUM!:0.00:#NUM!), (Biliary.tract.operation:0:4884:0:1:1.00:#NUM!:0.00:#NUM!), (Biliopancreatic.bypass:0:4884:0:1:1.00:#NUM!:0.00:#NUM!), (Bladder.ca lculus.removal:0:4884:0:1:1.00:#NUM!:0.00:#NUM!), (Bleeding.time.prolonged:0:4884:0:1:1.00: #NUM!:0.00:#NUM!), (Blood.alkaline.phosphatase:0:4884:0:1:1.00:#NUM!:0.00:#NUM!), (Blo od.chromogranin.A.increased:0:4884:0:1:1.00:#NUM!:0.00:#NUM!), (Blood.erythropoietin.incre ased:0:4884:0:1:1.00:#NUM!:0.00:#NUM!), (Blood.hyposmosis:0:4884:0:1:1.00:#NUM!:0.00:#NUM!), (Blood.mercury.abnormal:0:4884:0:1:1.00:#NUM!:0.00:#NUM!), (Blood.pregnenolone.i ncreased:0:4884:0:1:1.00:#NUM!:0.00:#NUM!), (Blood.pressure.orthostatic.decreased:0:4884:0: 1:1.00:#NUM!:0.00:#NUM!), (Blood.selenium. decreased:0:4884:0:1:1.00:#NUM!:0.00:#NUM!), (BLyS.polymorphism:0:4884:0:1:1.00:#NUM!:0.00:#NUM!), (Body.temperature.decreased:0:48 84:0:1:1.00:#NUM!:0.00:#NUM!), (Bone.marrow.donor:0:4884:0:1:1.00:#NUM!:0.00:#NUM!), (Bone.marrow.leukaemic.cell.infiltration:0:4884:0:1:1.00:#NUM!:0.00:#NUM!), (Bordetella.test. positive:0:4884:0:1:1.00:#NUM!:0.00:#NUM!), (Boredom:0:4884:0:1:1.00:#NUM!:0.00:#NUM!), (Brachytherapy.to.vagina:0:4884:0:1:1.00:#NUM!:0.00:#NUM!), (Brain.death:0:4884:0:1:1.00: #NUM!:0.00:#NUM!), (Brain.sarcoma:0:4884:0:1:1.00:#NUM!:0.00:#NUM!), (BRCA2.gene.mu tation.assay:0:4884:0:1:1.00:#NUM!:0.00:#NUM!), (Breast.adenoma:0:4884:0:1:1.00:#NUM!:0. 00:#NUM!), (Breast.fibroma:0:4884:0:1:1.00:#NUM!:0.00:#NUM!), (Breast.lump.removal:0:488 4:0:1:1.00:#NUM!:0.00:#NUM!), (Breast.sarcoma.metastatic:0:4884:0:1:1.00:#NUM!:0.00:#NUM!), (Breath.holding:0:4884:0:1:1.00:#NUM!:0.00:#NUM!), (Breech.presentation:0:4884:0:1:1.0 0:#NUM!:0.00:#NUM!), (Brief.psychotic.disorder.without.marked.stressors:0:4884:0:1:1.00:#N UM!:0.00:#NUM!), (Bronchial.prosthesis.insertion:0:4884:0:1:1.00:#NUM!:0.00:#NUM!), (Burk itt's.lymphoma.refractory:0:4884:0:1:1.00:#NUM!:0.00:#NUM!), (Bum. operation:0:4884:0:1:1.0 0:#NUM!:0.00:#NUM!), (Buming.sensation.mucosal:0:4884:0:1:1.00:#NUM!:0.00:#NUM!), (Bu rsa.disorder:0:4884:0:1:1.00:#NUM!:0.00:#NUM!), (Bursal.fluid.accumulation:0:4884:0:1:1.00:#NUM!:0.00:#NUM!), (CANDLE.syndrome:0:4884:0:1:1.00:#NUM!:0.00:#NUM!), (Capsular.co ntracture.associated.with.breast.implant:0:4884:0:1:1.00:#NUM!:0.00:#NUM!), (Carcinoma.in.sit u.of.skin:0:4884:0:1:1.00:#NUM!:0.00:#NUM!), (Cardiac.output.increased:0:4884:0:1:1.00:#NU M!:0.00:#NUM!), (Carotid.pulse. abnormal:0:4884:0:1:1.00:#NUM!:0.00:#NUM!), (Carotid.sinus.syndrome:0:4884:0:1:1.00:#NUM!:0.00:#NUM!), (Cartilage.graft:0:4884:0:1:1.00:#NUM!:0.00: #NUM!), (Cells.in.urine:0:4884:0:1:1.00:#NUM!:0.00:#NUM!), (Cellulitis.gangrenous:0:4884:0: 1:1.00:#NUM!:0.00:#NUM!), (Cementoplasty:0:4884:0:1:1.00:#NUM!:0.00:#NUM!), (Central.ne rvous.system.viral.infection:0:4884:0:1:1.00:#NUM!:0.00:#NUM!), (Central.venous.catheter.rem oval:0:4884:0:1:1.00:#NUM!:0.00:#NUM!), (Cerebellopontine.angle.tumour:0:4884:0:1:1.00:#N UM!:0.00:#NUM!), (Cerebrospinal.fluid. drainage:0:4884:0:1:1.00:#NUM!:0.00:#NUM!), (Cervic al.neuritis:0:4884:0:1:1.00:#NUM!:0.00:#NUM!), (Cervicitis.mycoplasmal:0:4884:0:1:1.00:#NU M!:0.00:#NUM!), (CHA2DS2-VASc-score:0:4884:0:1:1.00:#NUM!:0.00:#NUM!), (CHA2DS2-VASc.annual.stroke.risk.low:0:4884:0:1:1.00:#NUM!:0.00:#NUM!), (Chemical.bum.of.respirato ry.tract:0:4884:0:1:1.00:#NUM!:0.00:#NUM!), (Chemical.bums.of.eye:0:4884:0:1:1.00:#NUM!: 0.00:#NUM!), (Chemotherapeutic.drug.level:0:4884:0:1:1.00:#NUM!:0.00:#NUM!), (Chemother apy.extravasation.management:0:4884:0:1:1.00:#NUM!:0.00:#NUM!), (Chest.tube.insertion:0:48 84:0:1:1.00:#NUM!:0.00:#NUM!), (Choanal.stenosis:0:4884:0:1:1.00:#NUM!:0.00:#NUM!), (Ch olangiogram.abnormal:0:4884:0:1:1.00:#NUM!:0.00:#NUM!), (Chromosome.banding.abnormal: 0:4884:0:1:1.00:#NUM!:0.00:#NUM!), (Chronic.pulmonary.histoplasmosis:0:4884:0:1:1.00:#N UM!:0.00:#NUM!), (Ciliary.body.disorder:0:4884:0:1:1.00:#NUM!:0.00:#NUM!), (Coagulation.f actor.increased:0:4884:0:1:1.00:#NUM!:0.00:#NUM!), (Coagulation.factor.IX.level.abnormal:0: 4884:0:1:1.00:#NUM!:0.00:#NUM!), (Compensatory.sweating:0:4884:0:1:1.00:#NUM!:0.00:#N UM!), (Complication.of.delivery:0:4884:0:1:1.00:#NUM!:0.00:#NUM!), (Composite.valve.graft: 0:4884:0:1:1.00:#NUM!:0.00:#NUM!), (Compulsive.cheek.biting:0:4884:0:1:1.00:#NUM!:0.00: #NUM!), (Computerised.tomogram.breast.normal:0:4884:0:1:1.00:#NUM!:0.00:#NUM!), (Comp uterised.tomogram.coronary.artery.abnormal:0:4884:0:1:1.00:#NUM!:0.00:#NUM!), (Computeri sed.tomogram.normal:0:4884:0:1:1.00:#NUM!:0.00:#NUM!), (Concentric.sclerosis:0:4884:0:1:1. 00:#NUM!:0.00:#NUM!), (Confluent.and.reticulate.papillomatosis:0:4884:0:1:1.00:#NUM!:0.00: #NUM!), (Congenital.aortic.stenosis:0:4884:0:1:1.00:#NUM!:0.00:#NUM!), (Congenital.cyst:0:4 884:0:1:1.00:#NUM!:0.00:#NUM!), (Congenital.cystic.lung:0:4884:0:1:1.00:#NUM!:0.00:#NU M!), (Congenital.genital.malformation:0:4884:0:1:1.00:#NUM!:0.00:#NUM!), (Congenital.hearin g.disorder:0:4884:0:1:1.00:#NUM!:0.00:#NUM!), (Congenital.intestinal.malformation:0:4884:0: 1:1.00:#NUM!:0.00:#NUM!), (Congenital.jaw.malformation:0:4884:0:1:1.00:#NUM!:0.00:#NU M!), (Congenital.knee.deformity:0:4884:0:1:1.00:#NUM!:0.00:#NUM!), (Congenital.laryngeal.str idor:0:4884:0:1:1.00:#NUM!:0.00:#NUM!), (Congenital.lymphoedema:0:4884:0:1:1.00:#NUM!: 0.00:#NUM!), (Congenital.rubella.infection:0:4884:0:1:1.00:#NUM!:0.00:#NUM!), (Congenital. s yphilis:0:4884:0:1:1.00:#NUM!:0.00:#NUM!), (Congenital.tracheomalacia:0:4884:0:1:1.00:#NU M!:0.00:#NUM!), (Congenital.urethral.anomaly:0:4884:0:1:1.00:#NUM!:0.00:#NUM!), (Congeni tal.white.blood.cell.disorder:0:4884:0:1:1.00:#NUM!:0.00:#NUM!), (Conjunctival.abrasion:0:48 84:0:1:1.00:#NUM!:0.00:#NUM!), (Contact.lens.intolerance:0:4884:0:1:1.00:#NUM!:0.00:#NU M!), (Continuous.passive.motion.machine.therapy:0:4884:0:1:1.00:#NUM!:0.00:#NUM!), (Cor.tr iatriatum:0:4884:0:1:1.00:#NUM!:0.00:#NUM!), (Corynebacterium.bacteraemia:0:4884:0:1:1.00: #NUM!:0.00:#NUM!), (Costovertebral.angle.tenderness:0:4884:0:1:1.00:#NUM!:0.00:#NUM!), (Coxsackie.viral.infection:0:4884:0:1:1.00:#NUM!:0.00:#NUM!), (Cranial.nerve.infection:0:488 4:0:1:1.00:#NUM!:0.00:#NUM!), (Craniectomy:0:4884:0:1:1.00:#NUM!:0.00:#NUM!), (Creatini ne.renal.clearance.increased:0:4884:0:1:1.00:#NUM!:0.00:#NUM!), (Cystic.fibrosis.gastrointesti nal.disease:0:4884:0:1:1.00:#NUM!:0.00:#NUM!), (Dandy-Walker.syndrome:0:4884:0:1:1.00:#NUM!:0.00:#NUM!), (Defiant.behaviour:0:4884:0:1:1.00:#NUM!:0.00:#NUM!), (Deja.vu:0:4884:0:1:1.00:#NUM!:0.00:#NUM!), (Delusional.disorder,.mix ed.type:0:4884:0:1:1.00:#NUM!:0.00:#NUM!), (Demodicidosis:0:4884:0:1:1.00:#NUM!:0.00:#NUM!), (Dental.pulp.disorder:0:4884:0:1:1.00:#NUM!:0.00:#NUM!), (Dependence.on.respirator: 0:4884:0:1:1.00:#NUM!:0.00:#NUM!), (Dependent.personality.disorder:0:4884:0:1:1.00:#NUM!0.0.00:#NUM!), (Detrusor.sphincter.dyssynergia:0:4884:0:1:1.00:#NUM!:0.00:#NUM!), (Device.l oosening:0:4884:0:1:1.00:#NUM!:0.00:#NUM!), (Device.use.error:0:4884:0:1:1.00:#NUM!:0.00: #NUM!), (Diabetic.hyperosmolar. coma:0:4884:0:1:1.00:#NUM!:0.00:#NUM!), (Dialysis.efficac y.test:0:4884:0:1:1.00:#NUM!:0.00:#NUM!), (Diaphragmalgia:0:4884:0:1:1.00:#NUM!:0.00:#N UM!), (Diet.noncompliance:0:4884:0:1:1.00:#NUM!:0.00:#NUM!), (Diphtheria.carrier:0:4884:0: 1:1.00:#NUM!:0.00:#NUM!), (Disseminated.intravascular.coagulation.in.newborn:0:4884:0:1:1. 00:#NUM!:0.00:#NUM!), (Dolichocolon:0:4884:0:1:1.00:#NUM!:0.00:#NUM!), (Double.strande d. DNA. antibody.positive:0:4884:0:1:1.00:#NUM!:0.00:#NUM!), (Drug. dependence,.antepartum: 0:4884:0:1:1.00:#NUM!:0.00:#NUM!), (Drug.screen:0:4884:0:1:1.00:#NUM!:0.00:#NUM!), (Du puytren's.contracture.operation:0:4884:0:1:1.00:#NUM!:0.00:#NUM!), (Eales′.disease:0:4884:0:1:1.00:#NUM!:0.00:#NUM!), (Ear.tube.removal:0:4884:0:1:1.00:#NUM!:0.00:#NUM!), (Early.on set.familial.Alzheimer's.disease:0:4884:0:1:1.00:#NUM!:0.00:#NUM!), (Ectopic.antidiuretic.hor mone.secretion:0:4884:0:1:1.00:#NUM!:0.00:#NUM!), (Ectopic.thyroid:0:4884:0:1:1.00:#NUM!0.00:#NUM!), (Ejaculation.delayed:0:4884:0:1:1.00:#NUM!:0.00:#NUM!), (Encephalitis.califor nia:0:4884:0:1:1.00:#NUM!:0.00:#NUM!), (End.stage.AIDS:0:4884:0:1:1.00:#NUM!:0.00:#NU M!), (Endocrinopathy.diencephalic:0:4884:0:1:1.00:#NUM!:0.00:#NUM!), (Endoscopic.ultrasoun d:0:4884:0:1:1.00:#NUM!:0.00:#NUM!), (Enlarged.uvula:0:4884:0:1:1.00:#NUM!:0.00:#NUM!), (Enteroclysis:0:4884:0:1:1.00:#NUM!:0.00:#NUM!), (Enterocolonic.fistula:0:4884:0:1:1.00:#N UM!:0.00:#NUM!), (Eosinophilic.rhinitis:0:4884:0:1:1.00:#NUM!:0.00:#NUM!), (Epididymitis.t uberculous:0:4884:0:1:1.00:#NUM!:0.00:#NUM!), (Epiphyseal.fracture:0:4884:0:1:1.00:#NUM!: 0.00:#NUM!), (Exaggerated.startle.response:0:4884:0:1:1.00:#NUM!:0.00:#NUM!), (Excessive. eye.blinking:0:4884:0:1:1.00:#NUM!:0.00:#NUM!), (Exercise.electrocardiogram:0:4884:0:1:1.0 0:#NUM!:0.00:#NUM!), (Exposure.to.contaminated.air:0:4884:0:1:1.00:#NUM!:0.00:#NUM!), (Exposure.to.contaminated.water:0:4884:0:1:1.00:#NUM!:0.00:#NUM!), (Exposure.to.noise:0:48 84:0:1:1.00:#NUM!:0.00:#NUM!), (Exposure.via.skin.contact:0:4884:0:1:1.00:#NUM!:0.00:#N UM!), (Expulsion.of.medication:0:4884:0:1:1.00:#NUM!:0.00:#NUM!), (Extracorporeal.shock.w ave.therapy:0:4884:0:1:1.00:#NUM!:0.00:#NUM!), (Eye.complication.associated.with.device:0:4 884:0:1:1.00:#NUM!:0.00:#NUM!), (Eye.operation.complication:0:4884:0:1:1.00:#NUM!:0.00:#NUM!), (Eye.symptom:0:4884:0:1:1.00:#NUM!:0.00:#NUM!), (Eyelash.hyperpigmentation:0:48 84:0:1:1.00:#NUM!:0.00:#NUM!), (Eyelid.degenerative.disorder:0:4884:0:1:1.00:#NUM!:0.00:#NUM!), (Facial.asymmetry:0:4884:0:1:1.00:#NUM!:0.00:#NUM!), (Faecal.disimpaction:0:4884: 0:1:1.00:#NUM!:0.00:#NUM!), (Faecal.fat.increased:0:4884:0:1:1.00:#NUM!:0.00:#NUM!), (Fal lopian.tube.enlargement:0:4884:0:1:1.00:#NUM!:0.00:#NUM!), (Familial.hemiplegic. migraine:0:4884:0:1:1.00:#NUM!:0.00:#NUM!), (Fascial.rupture:0:4884:0:1:1.00:#NUM!:0.00:#NUM!), (F emale.genital.operation:0:4884:0:1:1.00:#NUM!:0.00:#NUM!), (Female.orgasmic.disorder:0:488 4:0:1:1.00:#NUM!:0.00:#NUM!), (Female.sexual.arousal.disorder:0:4884:0:1:1.00:#NUM!:0.00: #NUM!), (FEV1/FVC.ratio:0:4884:0:1:1.00:#NUM!:0.00:#NUM!), (Fibrin.D.dimer:0:4884:0:1:1. 00:#NUM!:0.00:#NUM!), (Foaming.at.mouth:0:4884:0:1:1.00:#NUM!:0.00:#NUM!), (Foetal.chr omosome. abnormality:0:4884:0:1:1.00:#NUM!:0.00:#NUM!), (Foetal.disorder:0:4884:0:1:1.00:#NUM!:0.00:#NUM!), (Foetal.distress.syndrome:0:4884:0:1:1.00:#NUM!:0.00:#NUM!), (Foetal.ta chyarrhythmia:0:4884:0:1:1.00:#NUM!:0.00:#NUM!), (Follicle.centre.lymphoma,.follicular.grad e.I,.II,.III.stage.II:0:4884:0:1:1.00:#NUM!:0.00:#NUM!), (Foreign.body.aspiration:0:4884:0:1:1. 00:#NUM!:0.00:#NUM!), (Frontal.sinus.operation:0:4884:0:1:1.00:#NUM!:0.00:#NUM!), (Fung al.paronychia:0:4884:0:1:1.00:#NUM!:0.00:#NUM!), (Galactography:0:4884:0:1:1.00:#NUM!:0. 00:#NUM!), (Gallbladder.neoplasm:0:4884:0:1:1.00:#NUM!:0.00:#NUM!), (Gambling:0:4884:0: 1:1.00:#NUM!:0.00:#NUM!), (Gas.poisoning:0:4884:0:1:1.00:#NUM!:0.00:#NUM!), (Gastric.va rices.haemorrhage:0:4884:0:1:1.00:#NUM!:0.00:#NUM!), (Gastrointestinal.disorder.congenital:0: 4884:0:1:1.00:#NUM!:0.00:#NUM!), (Gastrointestinal.polyp.haemorrhage:0:4884:0:1:1.00:#NU M!:0.00:#NUM!), (Gastroparesis.postoperative:0:4884:0:1:1.00:#NUM!:0.00:#NUM!), (Gastrosc hisis:0:4884:0:1:1.00:#NUM!:0.00:#NUM!), (Generalised.resistance.to.thyroid.hormone:0:4884: 0:1:1.00:#NUM!:0.00:#NUM!), (Genital.herpes.zoster:0:4884:0:1:1.00:#NUM!:0.00:#NUM!), (G enital.infection.bacterial:0:4884:0:1:1.00:#NUM!:0.00:#NUM!), (Genital.swelling:0:4884:0:1:1.0 0:#NUM!:0.00:#NUM!), (Gestational.age.test:0:4884:0:1:1.00:#NUM!:0.00:#NUM!), (Glassy.ey es:0:4884:0:1:1.00:#NUM!:0.00:#NUM!), (Gliosis:0:4884:0:1:1.00:#NUM!:0.00:#NUM!), (Glom erular.filtration.rate.abnormal:0:4884:0:1:1.00:#NUM!:0.00:#NUM!), (Glomerular.filtration.rate. normal:0:4884:0:1:1.00:#NUM!:0.00:#NUM!), (Glucagon.tolerance.test:0:4884:0:1:1.00:#NUM!: 0.00:#NUM!), (Graft.haemorrhage:0:4884:0:1:1.00:#NUM!:0.00:#NUM!), (Haematoma.evacuati on:0:4884:0:1:1.00:#NUM!:0.00:#NUM!), (Hair.growth.rate.abnormal:0:4884:0:1:1.00:#NUM!:0 0.00:#NUM!), (Hand-eye.coordination. impaired:0:4884:0:1:1.00:#NUM!:0.00:#NUM!), (Hand. repair.operation:0:4884: 0:1:1.00:#NUM!:0.00:#NUM!), (Harlequin.foetus:0:4884:0:1:1.00:#NUM!:0.00:#NUM!), (Head. and.neck.cancer.stage.I:0:4884:0:1:1.00:#NUM!:0.00:#NUM!), (Hepatic.adenoma:0:4884:0:1:1.0 0:#NUM!:0.00:#NUM!), (Hepatitis.B.surface.antigen:0:4884:0:1:1.00:#NUM!:0.00:#NUM!), (He patitis.C.antibody:0:4884:0:1:1.00:#NUM!:0.00:#NUM!), (Hepatitis.syphilitic:0:4884:0:1:1.00:#NUM!:0.00:#NUM!), (Herbal.toxicity:0:4884:0:1:1.00:#NUM!:0.00:#NUM!), (Hereditary.neurop athy.with.liability.to.pressure.palsies:0:4884:0:1:1.00:#NUM!:0.00:#NUM!), (Hereditary.papillar y.renal.carcinoma:0:4884:0:1:1.00:#NUM!:0.00:#NUM!), (Herpes.simplex.test.positive:0:4884:0 0.1:1.00:#NUM!:0.00:#NUM!), (Heterosexuality:0:4884:0:1:1.00:#NUM!:0.00:#NUM!), (High.de nsity.lipoprotein.normal:0:4884:0:1:1.00:#NUM!:0.00:#NUM!), (HIV.antigen.positive:0:4884:0: 1:1.00:#NUM!:0.00:#NUM!), (HIV.lipodystrophy:0:4884:0:1:1.00:#NUM!:0.00:#NUM!), (HIV.v iraemia:0:4884:0:1:1.00:#NUM!:0.00:#NUM!), (Holmes-Adie.pupil:0:4884:0:1:1.00:#NUM!:0.00:#NUM!), (Human.anti-human. antibody.test:0:4884:0:1:1.00:#NUM!:0.00:#NUM!), (Hydrocholecystis:0:4884:0:1:1.00: #NUM!:0.00:#NUM!), (Hydromyelia:0:4884:0:1:1.00:#NUM!:0.00:#NUM!), (Hyperexplexia:0:4 884:0:1:1.00:#NUM!:0.00:#NUM!), (Hypermetropia:0:4884:0:1:1.00:#NUM!:0.00:#NUM!), (Hy phaema:0:4884:0:1:1.00:#NUM!:0.00:#NUM!), (Hypogeusia:0:4884:0:1:1.00:#NUM!:0.00:#NU M!), (Hypoinsulinaemia:0:4884:0:1:1.00:#NUM!:0.00:#NUM!), (Hypotensive.anaesthesia.proced ure:0:4884:0:1:1.00:#NUM!:0.00:#NUM!), (Hypoventilation.neonatal:0:4884:0:1:1.00:#NUM!:0 0.00:#NUM!), (Ileal.fistula:0:4884:0:1:1.00:#NUM!:0.00:#NUM!), (Iliac.vein.occlusion:0:4884:0: 1:1.00:#NUM!:0.00:#NUM!), (Iliolumbar.syndrome:0:4884:0:1:1.00:#NUM!:0.00:#NUM!), (Illo gical.thinking:0:4884:0:1:1.00:#NUM!:0.00:#NUM!), (Immunotactoid.glomerulonephritis:0:488 4:0:1:1.00:#NUM!:0.00:#NUM!), (Implant.site.extravasation:0:4884:0:1:1.00:#NUM!:0.00:#NU M!), (Implant.site.hypoaesthesia:0:4884:0:1:1.00:#NUM!:0.00:#NUM!), (Imprisonment.of.relativ e:0:4884:0:1:1.00:#NUM!:0.00:#NUM!), (Incision.site.erythema:0:4884:0:1:1.00:#NUM!:0.00:#NUM!), (Increased.insulin.requirement:0:4884:0:1:1.00:#NUM!:0.00:#NUM!), (Infusion.site.ana esthesia:0:4884:0:1:1.00:#NUM!:0.00:#NUM!), (Inhibiting.antibodies.positive:0:4884:0:1:1.00:#NUM!:0.00:#NUM!), (Injection.site.discomfort:0:4884:0:1:1.00:#NUM!:0.00:#NUM!), (Injury.to.brachial.plexus.due.to.birth.trauma:0:4884:0:1:1.00:#NUM!:0.00:#NUM!), (Insulin.tolerance.tes t:0:4884:0:1:1.00:#NUM!:0.00:#NUM!), (Intercepted.drug.dispensing.error:0:4884:0:1:1.00:#NU M!:0.00:#NUM!), (Intercepted.medication.error:0:4884:0:1:1.00:#NUM!:0.00:#NUM!), (Interfero n.gamma.receptor.deficiency:0:4884:0:1:1.00:#NUM!:0.00:#NUM!), (Internal.limiting.membran e.peeling:0:4884:0:1:1.00:#NUM!:0.00:#NUM!), (Intervertebral.disc.calcification:0:4884:0:1:1.0 0:#NUM!:0.00:#NUM!), (Intervertebral.disc.space.narrowing:0:4884:0:1:1.00:#NUM!:0.00:#NU M!), (Intestinal.malrotation:0:4884:0:1:1.00:#NUM!:0.00:#NUM!), (Intestinal.pseudo-obstruction:0:4884:0:1:1.00:#NUM!:0.00:#NUM!), (Intestinal.varices:0:4884:0:1:1.00:#NUM!:0. 00:#NUM!), (Intra-abdominal.haematoma:0:4884:0:1:1.00:#NUM!:0.00:#NUM!), (Intracranial.haematoma:0:4884:0 0.1:1.00:#NUM!:0.00:#NUM!), (Intraspinal.abscess:0:4884:0:1:1.00:#NUM!:0.00:#NUM!), (Irreg ular.sleep.phase:0:4884:0:1:1.00:#NUM!:0.00:#NUM!), (Jejunal.operation:0:4884:0:1:1.00:#NU M!:0.00:#NUM!), (Joint.abscess:0:4884:0:1:1.00:#NUM!:0.00:#NUM!), (Juvenile.melanoma.ben ign:0:4884:0:1:1.00:#NUM!:0.00:#NUM!), (Kabuki.make-up.syndrome:0:4884:0:1:1.00:#NUM!:0.00:#NUM!), (Lacrimal.gland.enlargement:0:4884:0:1:1. 00:#NUM!:0.00:#NUM!), (Lactation.puerperal.increased:0:4884:0:1:1.00:#NUM!:0.00:#NUM!), (Large.for.dates.baby:0:4884:0:1:1.00:#NUM!:0.00:#NUM!), (Laryngitis.bacterial:0:4884:0:1:1. 00:#NUM!:0.00:#NUM!), (Laryngo-onycho-cutaneous.syndrome:0:4884:0:1:1.00:#NUM!:0.00:#NUM!), (Laryngotracheitis.obstructive:0:488 4:0:1:1.00:#NUM!:0.00:#NUM!), (Left.ventricular.end-diastolic.pressure:0:4884:0:1:1.00:#NUM!:0.00:#NUM!), (Leukaemia.in.remission:0:4884:0:1:1. 00:#NUM!:0.00:#NUM!), (Ligneous.conjunctivitis:0:4884:0:1:1.00:#NUM!:0.00:#NUM!), (Limb.amputation:0:4884:0:1:1.00:#NUM!:0.00:#NUM!), (Limb.asymmetry:0:4884:0:1:1.00:#NUM!:0 0.00:#NUM!), (Limb.mass:0:4884:0:1:1.00:#NUM!:0.00:#NUM!), (Lip.and/or.oral.cavity.cancer.st age.IV:0:4884:0:1:1.00:#NUM!:0.00:#NUM!), (Lip.discolouration:0:4884:0:1:1.00:#NUM!:0.00: #NUM!), (Lip.haemorrhage:0:4884:0:1:1.00:#NUM!:0.00:#NUM!), (Lipase.decreased:0:4884:0:1 0.1.00:#NUM!:0.00:#NUM!), (Lipoma.excision:0:4884:0:1:1.00:#NUM!:0.00:#NUM!), (Liver.fun ction.test.decreased:0:4884:0:1:1.00:#NUM!:0.00:#NUM!), (Loop.electrosurgical.excision.proce dure:0:4884:0:1:1.00:#NUM!:0.00:#NUM!), (Lung.neoplasm.surgery:0:4884:0:1:1.00:#NUM!:0. 00:#NUM!), (Lymph.nodes.scan.abnormal:0:4884:0:1:1.00:#NUM!:0.00:#NUM!), (Lymphoid.le ukaemia. (in.remission):0:4884:0:1:1.00:#NUM!:0.00:#NUM!), (Lymphorrhoea:0:4884:0:1:1.00: #NUM!:0.00:#NUM!), (Lysozyme.decreased:0:4884:0:1:1.00:#NUM!:0.00:#NUM!), (Majeed's.s yndrome:0:4884:0:1:1.00:#NUM!:0.00:#NUM!), (Malassezia.infection:0:4884:0:1:1.00:#NUM!: 0.00:#NUM!), (Malignant.cranial.nerve.neoplasm:0:4884:0:1:1.00:#NUM!:0.00:#NUM!), (Malig nant.lymphoma.unclassifiable.high.grade:0:4884:0:1:1.00:#NUM!:0.00:#NUM!), (Malignant.mu scle.neoplasm:0:4884:0:1:1.00:#NUM!:0.00:#NUM!), (Malignant.neoplasm.of.orbit:0:4884:0:1:1 0.00:#NUM!:0.00:#NUM!), (Malignant.neoplasm.of.placenta:0:4884:0:1:1.00:#NUM!:0.00:#NU M!), (Malignant.neoplasm.of.retina:0:4884:0:1:1.00:#NUM!:0.00:#NUM!), (Malignant.neoplasm. of.seminal.vesicle:0:4884:0:1:1.00:#NUM!:0.00:#NUM!), (Marginal.zone.lymphoma.recurrent:0: 4884:0:1:1.00:#NUM!:0.00:#NUM!), (Maxillofacial.operation:0:4884:0:1:1.00:#NUM!:0.00:#N UM!), (Mean.cell.haemoglobin.concentration:0:4884:0:1:1.00:#NUM!:0.00:#NUM!), (Medical.co unselling:0:4884:0:1:1.00:#NUM!:0.00:#NUM!), (Medical.device.site.inflammation:0:4884:0:1:1 0.00:#NUM!:0.00:#NUM!), (Meningioma.surgery:0:4884:0:1:1.00:#NUM!:0.00:#NUM!), (Menin gism:0:4884:0:1:1.00:#NUM!:0.00:#NUM!), (Meningitis.borrelia:0:4884:0:1:1.00:#NUM!:0.00: #NUM!), (Meningitis.toxoplasmal:0:4884:0:1:1.00:#NUM!:0.00:#NUM!), (Meningococcal.bacter aemia:0:4884:0:1:1.00:#NUM!:0.00:#NUM!), (Meningococcal.carditis:0:4884:0:1:1.00:#NUM!: 0.00:#NUM!), (Meningococcal.sepsis:0:4884:0:1:1.00:#NUM!:0.00:#NUM!), (Meningomyelitis.h erpes:0:4884:0:1:1.00:#NUM!:0.00:#NUM!), (Mesenteric.venous.occlusion:0:4884:0:1:1.00:#N UM!:0.00:#NUM!), (Metabolic.function.test:0:4884:0:1:1.00:#NUM!:0.00:#NUM!), (Metastases. to.fallopian.tube:0:4884:0:1:1.00:#NUM!:0.00:#NUM!), (Metastases.to.larynx:0:4884:0:1:1.00:#NUM!:0.00:#NUM!), (Metastases.to.perineum:0:4884:0:1:1.00:#NUM!:0.00:#NUM!), (Metastase s.to.urinary.tract:0:4884:0:1:1.00:#NUM!:0.00:#NUM!), (Microbiology.test.abnormal:0:4884:0:1: 1.00:#NUM!:0.00:#NUM!), (Microwave.therapy:0:4884:0:1:1.00:#NUM!:0.00:#NUM!), (Migrai ne-triggered.seizure:0:4884:0:1:1.00:#NUM!:0.00:#NUM!), (Mitral.valve.atresia:0:4884:0:1:1.00:#NUM!:0.00:#NUM!), (Moaning:0:4884:0:1:1.00:#NUM!:0.00:#NUM!), (Moebius.II.syndrome:0: 4884:0:1:1.00:#NUM!:0.00:#NUM!), (Monoclonal.antibody.unconjugated.therapy:0:4884:0:1:1. 00:#NUM!:0.00:#NUM!), (Monocytosis:0:4884:0:1:1.00:#NUM!:0.00:#NUM!), (Morbid.thought s:0:4884:0:1:1.00:#NUM!:0.00:#NUM!), (Mouth.cyst:0:4884:0:1:1.00:#NUM!:0.00:#NUM!), (M ucocutaneous.ulceration:0:4884:0:1:1.00:#NUM!:0.00:#NUM!), (Mucopolysaccharidosis.III:0:48 84:0:1:1.00:#NUM!:0.00:#NUM!), (Muir-Torre.syndrome:0:4884:0:1:1.00:#NUM!:0.00:#NUM!), (Multiple.cardiac.defects:0:4884:0:1:1.0 0:#NUM!:0.00:#NUM!), (Multiple.endocrine.neoplasia.Type.2A:0:4884:0:1:1.00:#NUM!:0.00:#NUM!), (Multiple.pregnancy:0:4884:0:1:1.00:#NUM!:0.00:#NUM!), (Muscle.relaxant.drug.level. therapeutic:0:4884:0:1:1.00:#NUM!:0.00:#NUM!), (Myeloblastoma:0:4884:0:1:1.00:#NUM!:0.0 0:#NUM!), (Narrow.anterior.chamber.angle:0:4884:0:1:1.00:#NUM!:0.00:#NUM!), (Nasal.crusti ng:0:4884:0:1:1.00:#NUM!:0.00:#NUM!), (Nasal.turbinate.abnormality:0:4884:0:1:1.00:#NUM!: 0.00:#NUM!), (Nasal.vestibulitis:0:4884:0:1:1.00:#NUM!:0.00:#NUM!), (Natural.killer.T.cell.co unt.decreased:0:4884:0:1:1.00:#NUM!:0.00:#NUM!), (Neonatal.hyponatraemia:0:4884:0:1:1.00: #NUM!:0.00:#NUM!), (Neonatal.lupus.erythematosus:0:4884:0:1:1.00:#NUM!:0.00:#NUM!), (N erve.root.injury.cervical:0:4884:0:1:1.00:#NUM!:0.00:#NUM!), (Neurectomy:0:4884:0:1:1.00:#NUM!:0.00:#NUM!), (Neurocryptococcosis:0:4884:0:1:1.00:#NUM!:0.00:#NUM!), (Neuronal.ne uropathy:0:4884:0:1:1.00:#NUM!:0.00:#NUM!), (Neurovascular.conflict:0:4884:0:1:1.00:#NUM!: 0.00:#NUM!), (Neutrophil.function.test.abnormal:0:4884:0:1:1.00:#NUM!:0.00:#NUM!), (Nipp le.infection:0:4884:0:1:1.00:#NUM!:0.00:#NUM!), (Nodal.osteoarthritis:0:4884:0:1:1.00:#NUM!: 0.00:#NUM!), (Nodular.lymphocyte.predominant.Hodgkin.lymphoma:0:4884:0:1:1.00:#NUM!: 0.00:#NUM!), (Noninfective.conjunctivitis:0:4884:0:1:1.00:#NUM!:0.00:#NUM!), (Noninfective. oophoritis:0:4884:0:1:1.00:#NUM!:0.00:#NUM!), (Nonkeratinising.carcinoma.of.nasopharynx:0: 4884:0:1:1.00:#NUM!:0.00:#NUM!), (Normal.pressure.hydrocephalus:0:4884:0:1:1.00:#NUM!: 0.00:#NUM!), (Nuchal.rigidity:0:4884:0:1:1.00:#NUM!:0.00:#NUM!), (Nuclear.magnetic.resona nce.imaging.spinal.normal:0:4884:0:1:1.00:#NUM!:0.00:#NUM!), (Nuclear.magnetic.resonance.i maging.whole.body:0:4884:0:1:1.00:#NUM!:0.00:#NUM!), (Oesophageal.carcinoma.stage.0:0:4 884:0:1:1.00:#NUM!:0.00:#NUM!), (Oesophagram:0:4884:0:1:1.00:#NUM!:0.00:#NUM!), (Oest radiol.decreased:0:4884:0:1:1.00:#NUM!:0.00:#NUM!), (Oncocytoma:0:4884:0:1:1.00:#NUM!:0 0.00:#NUM!), (Opiates.positive:0:4884:0:1:1.00:#NUM!:0.00:#NUM!), (Opisthorchiasis:0:4884:0: 1:1.00:#NUM!:0.00:#NUM!), (Oral.mucosa.haematoma:0:4884:0:1:1.00:#NUM!:0.00:#NUM!), (Oral.pustule:0:4884:0:1:1.00:#NUM!:0.00:#NUM!), (Orbital.granuloma:0:4884:0:1:1.00:#NUM!: 0.00:#NUM!), (Oropharyngeal.cancer.stage.III:0:4884:0:1:1.00:#NUM!:0.00:#NUM!), (Orophar yngeal.lymphoepithelioma:0:4884:0:1:1.00:#NUM!:0.00:#NUM!), (Oroticaciduria:0:4884:0:1:1. 00:#NUM!:0.00:#NUM!), (Osteochondroma:0:4884:0:1:1.00:#NUM!:0.00:#NUM!), (Osteoma:0: 4884:0:1:1.00:#NUM!:0.00:#NUM!), (Ovarian.enlargement:0:4884:0:1:1.00:#NUM!:0.00:#NU M!), (Overprotective.parent:0:4884:0:1:1.00:#NUM!:0.00:#NUM!), (Pacemaker.generated.arrhyt hmia:0:4884:0:1:1.00:#NUM!:0.00:#NUM!), (Paget's.disease.of.the.vulva:0:4884:0:1:1.00:#NU M!:0.00:#NUM!), (Palatal.operation:0:4884:0:1:1.00:#NUM!:0.00:#NUM!), (Palatectomy:0:4884: 0:1:1.00:#NUM!:0.00:#NUM!), (Palmar.erythema:0:4884:0:1:1.00:#NUM!:0.00:#NUM!), (Pancr eatitis.bacterial:0:4884:0:1:1.00:#NUM!:0.00:#NUM!), (Parakeratosis:0:4884:0:1:1.00:#NUM!:0 00:#NUM!), (Parasitic.test.positive:0:4884:0:1:1.00:#NUM!:0.00:#NUM!), (Parenteral.nutrition. associated.liver.disease:0:4884:0:1:1.00:#NUM!:0.00:#NUM!), (Patella.replacement:0:4884:0:1: 1.00:#NUM!:0.00:#NUM!), (Pathology.test:0:4884:0:1:1.00:#NUM!:0.00:#NUM!), (Pelvic.opera tion:0:4884:0:1:1.00:#NUM!:0.00:#NUM!), (Pelvic.prolapse:0:4884:0:1:1.00:#NUM!:0.00:#NU M!), (Penile.discharge:0:4884:0:1:1.00:#NUM!:0.00:#NUM!), (Penile.exfoliation:0:4884:0:1:1.00: #NUM!:0.00:#NUM!), (Penile.repair:0:4884:0:1:1.00:#NUM!:0.00:#NUM!), (Penile.swelling:0: 4884:0:1:1.00:#NUM!:0.00:#NUM!), (Peptic.ulcer.perforation:0:4884:0:1:1.00:#NUM!:0.00:#N UM!), (Perforation:0:4884:0:1:1.00:#NUM!:0.00:#NUM!), (Pericardial.effusion.malignant:0:488 4:0:1:1.00:#NUM!:0.00:#NUM!), (Pericardial.haemorrhage:0:4884:0:1:1.00:#NUM!:0.00:#NUM!), (Perinephric.collection:0:4884:0:1:1.00:#NUM!:0.00:#NUM!), (Peripheral.arteriogram:0:4884: 0:1:1.00:#NUM!:0.00:#NUM!), (Peripheral.endarterectomy:0:4884:0:1:1.00:#NUM!:0.00:#NUM!), (Peripheral.nervous.system.neoplasm:0:4884:0:1:1.00:#NUM!:0.00:#NUM!), (Peripheral.paral ysis:0:4884:0:1:1.00:#NUM!:0.00:#NUM!), (Peritoneal.cyst:0:4884:0:1:1.00:#NUM!:0.00:#NU M!), (Peritoneal.dialysis.complication:0:4884:0:1:1.00:#NUM!:0.00:#NUM!), (Phakomatosis:0:4 884:0:1:1.00:#NUM!:0.00:#NUM!), (Phlebolith:0:4884:0:1:1.00:#NUM!:0.00:#NUM!), (Photody namic.diagnostic.procedure:0:4884:0:1:1.00:#NUM!:0.00:#NUM!), (Pineal.gland.cyst:0:4884:0:1: 1.00:#NUM!:0.00:#NUM!), (Pitted.keratolysis:0:4884:0:1:1.00:#NUM!:0.00:#NUM!), (Pityriasis.alba:0:4884:0:1:1.00:#NUM!:0.00:#NUM!), (Placental.neoplasm:0:4884:0:1:1.00:#NUM!:0.00: #NUM!), (Plasma.viscosity.abnormal:0:4884:0:1:1.00:#NUM:0.00:#NUM!), (Plasminogen.activ ator.inhibitor:0:4884:0:1:1.00:#NUM!:0.00:#NUM!), (Pneumonia.bordetella:0:4884:0:1:1.00:#N UM!:0.00:#NUM!), (Pneumothorax.traumatic:0:4884:0:1:1.00:#NUM!:0.00:#NUM!), (PO2.decreased: 0:4884:0:1:1.00:#NUM!:0.00:#NUM!), (Polydactyly:0:4884:0:1:1.00:#NUM!:0.00:#NUM!), (Post.abortion.haemorrhage:0:4884:0:1:1.00:#NUM!:0.00:#NUM!), (Post.procedural.cellulitis:0: 4884:0:1:1.00:#NUM!:0.00:#NUM!), (Posterior.lens.capsulotomy:0:4884:0:1:1.00:#NUM!:0.00: #NUM!), (Postoperative.respiratory.failure:0:4884:0:1:1.00:#NUM!:0.00:#NUM!), (Potter's.syndr ome:0:4884:0:1:1.00:#NUM!:0.00:#NUM!), (Precursor.T-lymphoblastic.lymphoma/leukaemia.stage.I:0:4884:0:1:1.00:#NUM!:0.00:#NUM!), (Pregnancy.o n.contraceptive:0:4884:0:1:1.00:#NUM!:0.00:#NUM!), (Prenatal.screening.test:0:4884:0:1:1.00: #NUM!:0.00:#NUM!), (Prescribed.overdose:0:4884:0:1:1.00:#NUM!:0.00:#NUM!), (Primary.cer ebellar.degeneration:0:4884:0:1:1.00:#NUM!:0.00:#NUM!), (Product.adhesion.issue:0:4884:0:1: 1.00:#NUM!:0.00:#NUM!), (Product.commingling:0:4884:0:1:1.00:#NUM!:0.00:#NUM!), (Prod uct.label.on.wrong.product:0:4884:0:1:1.00:#NUM!:0.00:#NUM!), (Product.physical.issue:0:488 4:0:1:1.00:#NUM!:0.00:#NUM!), (Product.taste.abnormal:0:4884:0:1:1.00:#NUM!:0.00:#NUM!), (Progressive.external.ophthalmoplegia:0:4884:0:1:1.00:#NUM!:0.00:#NUM!), (Protein.induced.by.vitamin.K.absence.or.antagonist.II.increased:0:4884:0:1:1.00:#NUM!:0.00:#NUM!), (Pseudo dementia:0:4884:0:1:1.00:#NUM!:0.00:#NUM!), (Pseudopapilloedema:0:4884:0:1:1.00:#NUM!: 0.00:#NUM!), (Pseudosarcoma:0:4884:0:1:1.00:#NUM!:0.00:#NUM!), (Psychiatric.investigation: 0:4884:0:1:1.00:#NUM!:0.00:#NUM!), (Psychosis.postoperative:0:4884:0:1:1.00:#NUM!:0.00:#NUM!), (Psychosocial.support:0:4884:0:1:1.00:#NUM!:0.00:#NUM!), (Pudendal.block:0:4884:0: 1:1.00:#NUM!:0.00:#NUM!), (Pulmonary.alveolar.microlithiasis:0:4884:0:1:1.00:#NUM!:0.00:#NUM!), (Puncture.site.haemorrhage:0:4884:0:1:1.00:#NUM!:0.00:#NUM!), (Puncture.site.infecti on:0:4884:0:1:1.00:#NUM!:0.00:#NUM!), (Pupil.constriction.procedure:0:4884:0:1:1.00:#NUM!: 0.00:#NUM!), (Pyelocaliectasis:0:4884:0:1:1.00:#NUM!:0.00:#NUM!), (Pyelogram.retrograde:0: 4884:0:1:1.00:#NUM!:0.00:#NUM!), (Radial.head.dislocation:0:4884:0:1:1.00:#NUM!:0.00:#N UM!), (Radiation.alopecia:0:4884:0:1:1.00:#NUM!:0.00:#NUM!), (Radiation.mucositis:0:4884:0: 1:1.00:#NUM!:0.00:#NUM!), (Radiation.sickness.syndrome:0:4884:0:1:1.00:#NUM!:0.00:#NU M!), (Radical.neck.dissection:0:4884:0:1:1.00:#NUM!:0.00:#NUM!), (Radiotherapy.to.breast:0:4 884:0:1:1.00:#NUM!:0.00:#NUM!), (Radiotherapy.to.head.and.neck:0:4884:0:1:1.00:#NUM!:0.0 0:#NUM!), (Reaction.to.colouring:0:4884:0:1:1.00:#NUM!:0.00:#NUM!), (Reaction.to.food. addit ive:0:4884:0:1:1.00:#NUM!:0.00:#NUM!), (Rebound.effect:0:4884:0:1:1.00:#NUM!:0.00:#NU M!), (Red.blood.cells.urine:0:4884:0:1:1.00:#NUM!:0.00:#NUM!), (Reduced.facial.expression:0: 4884:0:1:1.00:#NUM!:0.00:#NUM!), (Refsum's.disease:0:4884:0:1:1.00:#NUM!:0.00:#NUM!), (Removal.of.foreign.body.from.eyelids:0:4884:0:1:1.00:#NUM!:0.00:#NUM!), (Renal.artery.arter iosclerosis:0:4884:0:1:1.00:#NUM!:0.00:#NUM!), (Renal.cancer.stage.II:0:4884:0:1:1.00:#NUM: 0.00:#NUM!), (Renal.tubular.dysfunction:0:4884:0:1:1.00:#NUM!:0.00:#NUM!), (Renal.tumou r.excision:0:4884:0:1:1.00:#NUM!:0.00:#NUM!), (Reproductive.hormone:0:4884:0:1:1.00:#NU M!:0.00:#NUM!), (Respiratory.dyskinesia:0:4884:0:1:1.00:#NUM!:0.00:#NUM!), (Respiratory.s yncytial.virus.test.positive:0:4884:0:1:1.00:#NUM!:0.00:#NUM!), (Reticuloendothelial.dysfuncti on:0:4884:0:1:1.00:#NUM!:0.00:#NUM!), (Retinal.implant:0:4884:0:1:1.00:#NUM!:0.00:#NUM!), (Retinopathy.congenital:0:4884:0:1:1.00:#NUM!:0.00:#NUM!), (Retinopathy.sickle.cell:0:488 4:0:1:1.00:#NUM!:0.00:#NUM!), (Rhinoscleroma:0:4884:0:1:1.00:#NUM!:0.00:#NUM!), (Rigid. hymen:0:4884:0:1:1.00:#NUM!:0.00:#NUM!), (Salivary.gland.adenoma:0:4884:0:1:1.00:#NUM!: 0.00:#NUM!), (Salpingo-oophoritis:0:4884:0:1:1.00:#NUM!:0.00:#NUM!), (Scan.parathyroid:0:4884:0:1:1.00:#NUM!:0.0 0:#NUM!), (Scan.spleen:0:4884:0:1:1.00:#NUM!:0.00:#NUM!), (Schizencephaly:0:4884:0:1:1.0 0:#NUM!:0.00:#NUM!), (Scleral.buckling.surgery:0:4884:0:1:1.00:#NUM!:0.00:#NUM!), (Scrot al.disorder:0:4884:0:1:1.00:#NUM!:0.00:#NUM!), (Scrotal.irritation:0:4884:0:1:1.00:#NUM!:0.0 0:#NUM!), (Selective.eating.disorder:0:4884:0:1:1.00:#NUM!:0.00:#NUM!), (Serratia.test.positiv e:0:4884:0:1:1.00:#NUM!:0.00:#NUM!), (Sertoli-cell-only.syndrome:0:4884:0:1:1.00:#NUM!:0.00:#NUM!), (Shared.psychotic.disorder:0:4884:0:1:1.0 0:#NUM!:0.00:#NUM!), (Sigmoidoscopy.abnormal:0:4884:0:1:1.00:#NUM!:0.00:#NUM!), (Sinu s.tarsi.syndrome:0:4884:0:1:1.00:#NUM!:0.00:#NUM!), (Skin.adhesion:0:4884:0:1:1.00:#NUM!: 0.00:#NUM!), (Skin.cyst.excision:0:4884:0:1:1.00:#NUM!:0.00:#NUM!), (Skin.implant.removal: 0:4884:0:1:1.00:#NUM!:0.00:#NUM!), (Skin.lesion.removal:0:4884:0:1:1.00:#NUM!:0.00:#NU M!), (Sleep.disorder.due.to.general.medical.condition,.hypersomnia.type:0:4884:0:1:1.00:#NUM!: 0.00:#NUM!), (Smith-Lemli-Opitz.syndrome:0:4884:0:1:1.00:#NUM!:0.00:#NUM!), (Soft.tissue.atrophy:0:4884:0:1:1.00:#N UM!:0.00:#NUM!), (Soliloquy:0:4884:0:1:1.00:#NUM!:0.00:#NUM!), (Speech.sound.disorder:0: 4884:0:1:1.00:#NUM!:0.00:#NUM!), (Spinal.subdural.haemorrhage:0:4884:0:1:1.00:#NUM!:0.0 0:#NUM!), (Spirochaetal.infection:0:4884:0:1:1.00:#NUM!:0.00:#NUM!), (Splenic.cyst:0:4884:0: 1:1.00:#NUM!:0.00:#NUM!), (Splenic.embolism:0:4884:0:1:1.00:#NUM!:0.00:#NUM!), (Spleni c.injury:0:4884:0:1:1.00:#NUM!:0.00:#NUM!), (Spondyloepiphyseal.dysplasia:0:4884:0:1:1.00: #NUM!:0.00:#NUM!), (Stapedectomy:0:4884:0:1:1.00:#NUM!:0.00:#NUM!), (Stoma.closure:0:4 884:0:1:1.00:#NUM!:0.00:#NUM!), (Stoma.obstruction:0:4884:0:1:1.00:#NUM!:0.00:#NUM!), (Stomach.mass:0:4884:0:1:1.00:#NUM!:0.00:#NUM!), (Strabismus.congenital:0:4884:0:1:1.00:#NUM!:0.00:#NUM!), (Stress.polycythaemia:0:4884:0:1:1.00:#NUM!:0.00:#NUM!), (Struck.by.li ghtning:0:4884:0:1:1.00:#NUM!:0.00:#NUM!), (Subdural.haematoma.evacuation:0:4884:0:1:1.0 0:#NUM!:0.00:#NUM!), (Submandibular. mass:0:4884:0:1:1.00:#NUM!:0.00:#NUM!), (Subvalvu lar.aortic.stenosis:0:4884:0:1:1.00:#NUM!:0.00:#NUM!), (Sudden. cardiac. death:0:4884:0:1:1.00: #NUM!:0.00:#NUM!), (Suicide.threat:0:4884:0:1:1.00:#NUM!:0.00:#NUM!), (Superior.vena.cav a.stenosis:0:4884:0:1:1.00:#NUM!:0.00:#NUM!), (Superovulation:0:4884:0:1:1.00:#NUM!:0.00: #NUM!), (Suture.removal:0:4884:0:1:1.00:#NUM!:0.00:#NUM!), (Sympathectomy:0:4884:0:1:1. 00:#NUM!:0.00:#NUM!), (TEMPI.syndrome:0:4884:0:1:1.00:#NUM!:0.00:#NUM!), (Tendon.sh eath.disorder:0:4884:0:1:1.00:#NUM!:0.00:#NUM!), (Therapeutic.response.shortened:0:4884:0:1 0.1.00:#NUM!:0.00:#NUM!), (Thought.insertion:0:4884:0:1:1.00:#NUM!:0.00:#NUM!), (Thrombi n.time.abnormal:0:4884:0:1:1.00:#NUM!:0.00:#NUM!), (Thyroglobulin.increased:0:4884:0:1:1.0 0:#NUM!:0.00:#NUM!), (Thyroid.calcification:0:4884:0:1:1.00:#NUM!:0.00:#NUM!), (Tilt.table.test:0:4884:0:1:1.00:#NUM!:0.00:#NUM!), (Tongue.carcinoma.stage.III:0:4884:0:1:1.00:#NUM!: 0.00:#NUM!), (Tongue.injury:0:4884:0:1:1.00:#NUM!:0.00:#NUM!), (Tooth.avulsion:0:4884:0: 1:1.00:#NUM!:0.00:#NUM!), (Trachoma:0:4884:0:1:1.00:#NUM!:0.00:#NUM!), (Transferrin.sa turation.increased:0:4884:0:1:1.00:#NUM!:0.00:#NUM!), (Transmission.of.an.infectious.agent.vi a.product:0:4884:0:1:1.00:#NUM!:0.00:#NUM!), (Transurethral.incision.of.prostate:0:4884:0:1:1 0.00:#NUM!:0.00:#NUM!), (Tropical.infectious.disease:0:4884:0:1:1.00:#NUM!:0.00:#NUM!), (T ype.I.hyperlipidaemia:0:4884:0:1:1.00:#NUM!:0.00:#NUM!), (Typhus:0:4884:0:1:1.00:#NUM!: 0.00:#NUM!), (Tyrosine.kinase.mutation.assay:0:4884:0:1:1.00:#NUM!:0.00:#NUM!), (Ultrasoni c.angiogram:0:4884:0:1:1.00:#NUM!:0.00:#NUM!), (Ultrasound.abdomen:0:4884:0:1:1.00:#NU M!:0.00:#NUM!), (Ultrasound.abdomen.abnormal:0:4884:0:1:1.00:#NUM!:0.00:#NUM!), (Ultras ound.kidney.abnormal:0:4884:0:1:1.00:#NUM!:0.00:#NUM!), (Ultrasound.ovary:0:4884:0:1:1.0 0:#NUM!:0.00:#NUM!), (Ultrasound.prostate.abnormal:0:4884:0:1:1.00:#NUM!:0.00:#NUM!), (Ultrasound. uterus.abnormal:0:4884:0:1:1.00:#NUM!:0.00:#NUM!), (Upper. extremity. mass:0:48 84:0:1:1.00:#NUM!:0.00:#NUM!), (Urachal.abnormality:0:4884:0:1:1.00:#NUM!:0.00:#NUM!), (Urethral.atresia:0:4884:0:1:1.00:#NUM!:0.00:#NUM!), (Urethral.injury:0:4884:0:1:1.00:#NUM!: 0.00:#NUM!), (Urethral.intrinsic.sphincter.deficiency:0:4884:0:1:1.00:#NUM!:0.00:#NUM!), (Urethral.stent.removal:0:4884:0:1:1.00:#NUM!:0.00:#NUM!), (Urethritis.mycoplasmal:0:4884:0 0.1:1.00:#NUM!:0.00:#NUM!), (Urinary.cystectomy:0:4884:0:1:1.00:#NUM!:0.00:#NUM!), (Urin ary.incontinence.surgery:0:4884:0:1:1.00:#NUM!:0.00:#NUM!), (Urinary.straining:0:4884:0:1:1. 00:#NUM!:0.00:#NUM!), (Urine.analysis:0:4884:0:1:1.00:#NUM!:0.00:#NUM!), (Usher's.syndr ome:0:4884:0:1:1.00:#NUM!:0.00:#NUM!), (Uterine.adhesions:0:4884:0:1:1.00:#NUM!:0.00:#NUM!), (Uterine.fibrosis:0:4884:0:1:1.00:#NUM!:0.00:#NUM!), (Uterine.injury:0:4884:0:1:1.00: #NUM!:0.00:#NUM!), (Uterine.inversion:0:4884:0:1:1.00:#NUM!:0.00:#NUM!), (Uterine.irrigati on:0:4884:0:1:1.00:#NUM!:0.00:#NUM!), (Uterine.polypectomy:0:4884:0:1:1.00:#NUM!:0.00:#NUM!), (Uterine.scar:0:4884:0:1:1.00:#NUM!:0.00:#NUM!), (Uterine.stenosis:0:4884:0:1:1.00:#NUM!:0.00:#NUM!), (Vaccination.site.discomfort:0:4884:0:1:1.00:#NUM!:0.00:#NUM!), (Vagi nal.cancer.stage.III:0:4884:0:1:1.00:#NUM!:0.00:#NUM!), (Vaginal.cyst:0:4884:0:1:1.00:#NUM: 0.00:#NUM!), (Vascular.access.malfunction:0:4884:0:1:1.00:#NUM!:0.00:#NUM!), (Vascular.i maging:0:4884:0:1:1.00:#NUM!:0.00:#NUM!), (Vascular.injury:0:4884:0:1:1.00:#NUM!:0.00:#NUM!), (Vascular.puncture.site.sealing:0:4884:0:1:1.00:#NUM!:0.00:#NUM!), (Vascular.test:0:4 884:0:1:1.00:#NUM!:0.00:#NUM!), (Vasectomy:0:4884:0:1:1.00:#NUM!:0.00:#NUM!), (Vector-bome.transmission.of.infection:0:4884:0:1:1.00:#NUM!:0.00:#NUM!), (Venomous.sting:0:4884: 0:1:1.00:#NUM!:0.00:#NUM!), (Ventilation.perfusion.mismatch:0:4884:0:1:1.00:#NUM!:0.00:#NUM!), (Ventricle.rupture:0:4884:0:1:1.00:#NUM!:0.00:#NUM!), (Ventriculo-vascular. shunt:0:4884:0:1:1.00:#NUM!:0.00:#NUM!), (Vesical.fistula:0:4884:0:1:1.00:#NUM!:0 0.00:#NUM!), (Victim.of.chemical.submission:0:4884:0:1:1.00:#NUM!:0.00:#NUM!), (VIIIth.ner ve.lesion:0:4884:0:1:1.00:#NUM!:0.00:#NUM!), (Viral.pericarditis:0:4884:0:1:1.00:#NUM!:0.00: #NUM!), (Viral.tonsillitis:0:4884:0:1:1.00:#NUM!:0.00:#NUM!), (Visceral.larva.migrans:0:4884: 0:1:1.00:#NUM!:0.00:#NUM!), (VIth.nerve.paralysis:0:4884:0:1:1.00:#NUM!:0.00:#NUM!), (V ocal.cord.paresis:0:4884:0:1:1.00:#NUM!:0.00:#NUM!), (Vulvovaginitis.streptococcal:0:4884:0: 1:1.00:#NUM!:0.00:#NUM!), (Wrist. deformity:0:4884:0:1:1.00:#NUM!:0.00:#NUM!), (X-ray.limb:0:4884:0:1:1.00:#NUM!:0.00:#NUM!), (X-ray.limb.abnormal:0:4884:0:1:1.00:#NUM!:0.00:#NUM!), (Xanthelasma:0:4884:0:1:1.00:#NUM!: 0.00:#NUM!), (Yaws:0:4884:0:1:1.00:#NUM!:0.00:#NUM!), (B-lymphocyte.count.decreased:6:4878:0:1:1.00:1.00:0.86:0.92), (Intestinal.atresia:5:4879:0:1:1.00: 1.00:0.83:0.91), (Primary.mediastinal.large.B-cell.lymphoma.stage.IV:5:4879:0:1:1.00:1.00:0.83:0.91), (Choroid.tubercles:4:4880:0:1:1.00:1.0 0:0.80:0.89), (Gastrointestinal.lymphoma:4:4880:0:1:1.00:1.00:0.80:0.89), (Follicle.centre.lymph oma.diffuse.small.cell.lymphoma.stage.III:3:4881:0:1:1.00:1.00:0.75:0.86), (Hodgkin's.disease.m ixed.cellularity.stage.I.site.unspecified:3:4881:0:1:1.00:1.00:0.75:0.86), (Marburg's.variant.multi ple.sclerosis:3:4881:0:1:1.00:1.00:0.75:0.86), (Neurofibrosarcoma:3:4881:0:1:1.00:1.00:0.75:0.8 6), (Ovarian.dysgerminoma.stage.unspecified:3:4881:0:1:1.00:1.00:0.75:0.86), (Ovarian.germ.cell.endodermal.sinus.tumour.stage.I:3:4881:0:1:1.00:1.00:0.75:0.86), (Patemal.exposure.during.preg nancy:3:4881:0:1:1.00:1.00:0.75:0.86), (Peripheral.primitive.neuroectodermal.tumour.of.soft.tiss ue:3:4881:0:1:1.00:1.00:0.75:0.86), (Peritoneal.gliomatosis:3:4881:0:1:1.00:1.00:0.75:0.86), (Retr operitoneal.lymphadenopathy:3:4881:0:1:1.00:1.00:0.75:0.86), (Silicotuberculosis:3:4881:1:0:1.0 0:0.75:1.00:0.86), (Testicular.embryonal.carcinoma.stage.II:3:4881:0:1:1.00:1.00:0.75:0.86), (Ant erograde.amnesia:2:4882:0:1:1.00:1.00:0.67:0.80), (Anti-GAD.antibody.positive:2:4882:0:1:1.00:1.00:0.67:0.80), (Anti-glycyl-tRNA.synthetase.antibody.positive:2:4882:0:1:1.00:1.00:0.67:0.80), (Arteriovenous.graft:2:4882: 0:1:1.00:1.00:0.67:0.80), (Critical.illness:2:4882:0:1:1.00:1.00:0.67:0.80), (Diaphragmatic.injury: 2:4882:0:1:1.00:1.00:0.67:0.80), (Extragonadal.primary.germ.cell.tumour.mixed.stage.III:2:4882: 0:1:1.00:1.00:0.67:0.80), (Extragonadal.primary.seminoma. (pure).stage.II:2:4882:0:1:1.00:1.00:0 0.67:0.80), (Fatty.liver.alcoholic:2:4882:0:1:1.00:1.00:0.67:0.80), (Haemangioma.of.retina:2:4882: 0:1:1.00:1.00:0.67:0.80), (Hepatitis.B.e.antigen.positive:2:4882:0:1:1.00:1.00:0.67:0.80), (Heredit ary.non-polyposis.colorectal.cancer.syndrome:2:4882:0:1:1.00:1.00:0.67:0.80), (Human.chorionic.gonado tropin.increased:2:4882:0:1:1.00:1.00:0.67:0.80), (Infantile.vomiting:2:4882:0:1:1.00:1.00:0.67:0 0.80), (Intestinal.fibrosis:2:4882:0:1:1.00:1.00:0.67:0.80), (Melkersson-Rosenthal.syndrome:2:4882:0:1:1.00:1.00:0.67:0.80), (Metastases.to.testicle:2:4882:0:1:1.00:1.0 0:0.67:0.80), (Mucoepidermoid.carcinoma.of.salivary.gland:2:4882:0:1:1.00:1.00:0.67:0.80), (No ninfectious.myelitis:2:4882:0:1:1.00:1.00:0.67:0.80), (Ovarian.dysgerminoma.stage.II:2:4882:0:1: 1.00:1.00:0.67:0.80), (Ovarian.dysgerminoma.stage.III:2:4882:0:1:1.00:1.00:0.67:0.80), (Ovarian.germ.cell.cancer.stage.IV:2:4882:0:1:1.00:1.00:0.67:0.80), (Ovarian.germ.cell.endodermal.sinus. tumour.stage.II:2:4882:0:1:1.00:1.00:0.67:0.80), (Ovarian.germ.cell.endodermal.sinus.tumour.sta ge.III:2:4882:0:1:1.00:1.00:0.67:0.80), (Ovarian.germ.cell.teratoma.stage.III:2:4882:0:1:1.00:1.0 0:0.67:0.80), (Ovarian.rupture:2:4882:0:1:1.00:1.00:0.67:0.80), (Periprosthetic.fracture:2:4882:0: 1:1.00:1.00:0.67:0.80), (Radiation.retinopathy:2:4882:0:1:1.00:1.00:0.67:0.80), (Sexual.transmissi on.of.infection:2:4882:0:1:1.00:1.00:0.67:0.80), (Trisomy.9:2:4882:0:1:1.00:1.00:0.67:0.80), (Yel low.fever:2:4882:0:1:1.00:1.00:0.67:0.80), (Acquired.growth.hormone.resistance:1:4883:0:1:1.00: 1.00:0.50:0.67), (Acral.lentiginous.melanoma.stage.I:1:4883:0:1:1.00:1.00:0.50:0.67), (Acute.zon al.occult.outer.retinopathy:1:4883:0:1:1.00:1.00:0.50:0.67), (Alkaptonuria:1:4883:0:1:1.00:1.00:0 0.50:0.67), (Anti-aquaporin-4.antibody.positive:1:4883:0:1:1.00:1.00:0.50:0.67), (Atypical.femur.fracture:1:4883:0:1:1.00:1.0 0:0.50:0.67), (Benign.gastrointestinal.neoplasm:1:4883:0:1:1.00:1.00:0.50:0.67), (Bilateral.orchid ectomy:1:4883:0:1:1.00:1.00:0.50:0.67), (Biopsy.cervix:1:4883:0:1:1.00:1.00:0.50:0.67), (Biopsy. endometrium:1:4883:0:1:1.00:1.00:0.50:0.67), (Biopsy.peripheral.nerve:1:4883:0:1:1.00:1.00:0.5 0:0.67), (Body.height.increased:1:4883:0:1:1.00:1.00:0.50:0.67), (Body.mass.index:1:4883:0:1:1. 00:1.00:0.50:0.67), (Bone.fragmentation:1:4883:0:1:1.00:1.00:0.50:0.67), (Breast.enlargement:1:4 883:0:1:1.00:1.00:0.50:0.67), (Central.nervous.system.abscess:1:4883:0:1:1.00:1.00:0.50:0.67), (Cerebellar.hypoplasia:1:4883:0:1:1.00:1.00:0.50:0.67), (Chondroblastoma:1:4883:0:1:1.00:1.00:0 0.50:0.67), (Chondrodystrophy:1:4883:0:1:1.00:1.00:0.50:0.67), (Coagulation.factor.mutation:1:48 83:0:1:1.00:1.00:0.50:0.67), (Congenital.adrenal.gland.hypoplasia:1:4883:0:1:1.00:1.00:0.50:0.67), (Congenital.tricuspid.valve.atresia:1:4883:0:1:1.00:1.00:0.50:0.67), (Convulsion.in.childhood:1: 4883:0:1:1.00:1.00:0.50:0.67), (Cystadenocarcinoma.ovary:1:4883:0:1:1.00:1.00:0.50:0.67), (Dys acusis:1:4883:0:1:1.00:1.00:0.50:0.67), (Emergency.care.examination:1:4883:0:1:1.00:1.00:0.50: 0.67), (Endocardial.fibroelastosis:1:4883:0:1:1.00:1.00:0.50:0.67), (Endometrial.cancer.stage.II:1: 4883:0:1:1.00:1.00:0.50:0.67), (Eosinophilic.pneumonia.acute:1:4883:0:1:1.00:1.00:0.50:0.67), (E pidermal.necrosis:1:4883:0:1:1.00:1.00:0.50:0.67), (Extragonadal.primary.germ.cell.tumour.mixe d:1:4883:0:1:1.00:1.00:0.50:0.67), (Eyelid.thickening:1:4883:0:1:1.00:1.00:0.50:0.67), (Faciodigit ogenital.dysplasia:1:4883:0:1:1.00:1.00:0.50:0.67), (Factor.III.deficiency:1:4883:0:1:1.00:1.00:0. 50:0.67), (Femoral.artery.embolism:1:4883:0:1:1.00:1.00:0.50:0.67), (Fuchs′.syndrome:1:4883:0: 1:1.00:1.00:0.50:0.67), (Full.blood.count:1:4883:0:1:1.00:1.00:0.50:0.67), (Full.blood.count.incre ased:1:4883:0:1:1.00:1.00:0.50:0.67), (Genetic.polymorphism:1:4883:0:1:1.00:1.00:0.50:0.67), (G estational.age.test.abnormal:1:4883:0:1:1.00:1.00:0.50:0.67), (Glomerular.vascular.disorder:1:48 83:0:1:1.00:1.00:0.50:0.67), (Good.syndrome:1:4883:0:1:1.00:1.00:0.50:0.67), (Growth.accelerate d:1:4883:0:1:1.00:1.00:0.50:0.67), (Hearing.aid.therapy:1:4883:0:1:1.00:1.00:0.50:0.67), (Hepatiti s.B.DNA.increased:1:4883:0:1:1.00:1.00:0.50:0.67), (Hereditary.retinal.dystrophy:1:4883:0:1:1.0 0:1.00:0.50:0.67), (HLA-B.gene.status.assay:1:4883:0:1:1.00:1.00:0.50:0.67), (Hysterical.psychosis:1:4883:0:1:1.00:1.00: 0.50:0.67), (Imminent.abortion:1:4883:0:1:1.00:1.00:0.50:0.67), (Immunosuppressant.drug.level.i ncreased:1:4883:0:1:1.00:1.00:0.50:0.67), (Injection.site.vesicles:1:4883:0:1:1.00:1.00:0.50:0.67), (Interferon.therapy:1:4883:0:1:1.00:1.00:0.50:0.67), (Interstitial.granulomatous.dermatitis:1:488 3:0:1:1.00:1.00:0.50:0.67), (JC.virus.test:1:4883:0:1:1.00:1.00:0.50:0.67), (Joint.warmth:1:4883:0: 1:1.00:1.00:0.50:0.67), (Juvenile.Paget's.disease:1:4883:0:1:1.00:1.00:0.50:0.67), (Ketosis:1:488 3:0:1:1.00:1.00:0.50:0.67), (Large.cell.lung.cancer.stage.III:1:4883:0:1:1.00:1.00:0.50:0.67), (Leu kaemia.basophilic:1:4883:0:1:1.00:1.00:0.50:0.67), (Leukocoria:1:4883:0:1:1.00:1.00:0.50:0.67), (Lung.squamous.cell.carcinoma.stage.0:1:4883:0:1:1.00:1.00:0.50:0.67), (Lymphocyte.count.incr eased:1:4883:0:1:1.00:1.00:0.50:0.67), (MAGIC.syndrome:1:4883:0:1:1.00:1.00:0.50:0.67), (Mali gnant.neoplasm.of.spermatic.cord:1:4883:0:1:1.00:1.00:0.50:0.67), (Meningeal.repair:1:4883:0:1: 1.00:1.00:0.50:0.67), (Metastases.to.mouth:1:4883:0:1:1.00:1.00:0.50:0.67), (Morvan.syndrome:1:4883:0:1:1.00:1.00:0.50:0.67), (Mosaicism:1:4883:0:1:1.00:1.00:0.50:0.67), (Neck.plastic.surger y:1:4883:0:1:1.00:1.00:0.50:0.67), (Normal.labour:1:4883:0:1:1.00:1.00:0.50:0.67), (Oesophageal.fistula:1:4883:0:1:1.00:1.00:0.50:0.67), (Oligoasthenoteratozoospermia:1:4883:0:1:1.00:1.00:0.5 0:0.67), (Otitis.extema.fungal:1:4883:0:1:1.00:1.00:0.50:0.67), (Ovarian.cystectomy:1:4883:0:1:1. 00:1.00:0.50:0.67), (Ovarian.low.malignant.potential.tumour:1:4883:0:1:1.00:1.00:0.50:0.67), (Pa get's.disease.of.penis:1:4883:0:1:1.00:1.00:0.50:0.67), (Pancreatic.islets.hyperplasia:1:4883:0:1:1 0.00:1.00:0.50:0.67), (Paraneoplastic.encephalomyelitis:1:4883:0:1:1.00:1.00:0.50:0.67), (Parotide ctomy:1:4883:0:1:1.00:1.00:0.50:0.67), (Parvovirus.B19.infection:1:4883:0:1:1.00:1.00:0.50:0.67), (Peliosis.hepatis:1:4883:0:1:1.00:1.00:0.50:0.67), (Penile.blister:1:4883:0:1:1.00:1.00:0.50:0.67), (Penis.carcinoma.stage.IV:1:4883:0:1:1.00:1.00:0.50:0.67), (Perineal.ulceration:1:4883:0:1:1.00:1.00:0.50:0.67), (Poor.venous.access:1:4883:0:1:1.00:1.00:0.50:0.67), (Porphyrin.metabolism.dis order:1:4883:0:1:1.00:1.00:0.50:0.67), (Postoperative.renal.failure:1:4883:0:1:1.00:1.00:0.50:0.6 7), (Pregnancy.after.post.coital.contraception:1:4883:0:1:1.00:1.00:0.50:0.67), (Pregnancy.test.ne gative:1:4883:0:1:1.00:1.00:0.50:0.67), (Progressive.massive.fibrosis:1:4883:0:1:1.00:1.00:0.50:0 0.67), (Psychopathic.personality:1:4883:0:1:1.00:1.00:0.50:0.67), (Renal.vein.occlusion:1:4883:0:1: 1.00:1.00:0.50:0.67), (Retinitis.viral:1:4883:0:1:1.00:1.00:0.50:0.67), (Rhesus.antibodies.positive: 1:4883:0:1:1.00:1.00:0.50:0.67), (Rheumatoid.factor.quantitative.increased:1:4883:0:1:1.00:1.00: 0.50:0.67), (Rubinstein-Taybi.syndrome:1:4883:0:1:1.00:1.00:0.50:0.67), (Second.trimester.pregnancy:1:4883:0:1:1.00:1 0.00:0.50:0.67), (Serum.ferritin.abnormal:1:4883:0:1:1.00:1.00:0.50:0.67), (Skin.squamous.cell.car cinoma.metastatic:1:4883:0:1:1.00:1.00:0.50:0.67), (Somatic.hallucination:1:4883:0:1:1.00:1.00: 0.50:0.67), (Spinal.epidural.haematoma:1:4883:0:1:1.00:1.00:0.50:0.67), (Spontaneous.penile.ere ction:1:4883:0:1:1.00:1.00:0.50:0.67), (Stargardt's.disease:1:4883:0:1:1.00:1.00:0.50:0.67), (Subc utaneous.haematoma:1:4883:0:1:1.00:1.00:0.50:0.67), (Topography.comeal:1:4883:0:1:1.00:1.00: 0.50:0.67), (Transdermal.contraception:1:4883:0:1:1.00:1.00:0.50:0.67), (Transfusion.associated. graft.versus.host.disease:1:4883:0:1:1.00:1.00:0.50:0.67), (Type.1.lepra.reaction:1:4883:0:1:1.00: 1.00:0.50:0.67), (Ureteral.neoplasm:1:4883:0:1:1.00:1.00:0.50:0.67), (Ureteric.stenosis:1:4883:0: 1:1.00:1.00:0.50:0.67), (Urethral.valves:1:4883:0:1:1.00:1.00:0.50:0.67), (Varicose.veins.pelvic:1: 4883:0:1:1.00:1.00:0.50:0.67), (Vasculitic.rash:1:4883:0:1:1.00:1.00:0.50:0.67), (Viral.myositis:1: 4883:0:1:1.00:1.00:0.50:0.67), (21-hydroxylase.deficiency:0:4883:0:2:1.00:#NUM!:0.00:#NUM!), (Acanthosis:0:4883:0:2:1.00:#N UM!:0.00:#NUM!), (Activated.partial.thromboplastin.time:0:4883:0:2:1.00:#NUM!:0.00:#NUM!), (Adenoviral.conjunctivitis:0:4883:0:2:1.00:#NUM!:0.00:#NUM!), (Adrenal.gland.operation:0:4 883:0:2:1.00:#NUM!:0.00:#NUM!), (Adverse.food.reaction:0:4883:0:2:1.00:#NUM!:0.00:#NU M!), (Aeromonas.infection:0:4883:0:2:1.00:#NUM!:0.00:#NUM!), (Alcohol.use.disorder:0:4883: 0:2:1.00:#NUM!:0.00:#NUM!), (Alcoholic.hangover:0:4883:0:2:1.00:#NUM!:0.00:#NUM!), (All ergy.to.immunoglobulin.therapy:0:4883:0:2:1.00:#NUM!:0.00:#NUM!), (Allergy.to.sting:0:4883: 0:2:1.00:#NUM!:0.00:#NUM!), (Anal.cancer.stage.I:0:4883:0:2:1.00:#NUM!:0.00:#NUM!), (An giogram.peripheral:0:4883:0:2:1. 00:#NUM!:0.00:#NUM!), (Annular.elastolytic.giant.cell.granul oma:0:4883:0:2:1.00:#NUM!:0.00:#NUM!), (Anorectal.cellulitis:0:4883:0:2:1.00:#NUM!:0.00:#NUM!), (Anti-myelin-associated.glycoprotein.associated.polyneuropathy:0:4883:0:2:1.00:#NUM!:0.00:#NUM!), (Anti coagulation.drug.level.abnormal:0:4883:0:2:1.00:#NUM!:0.00:#NUM!), (Antimitochondrial.anti body:0:4883:0:2:1.00:#NUM!:0.00:#NUM!), (Antineutrophil.cytoplasmic.antibody.negative:0:48 83:0:2:1.00:#NUM!:0.00:#NUM!), (Aortic. valve.sclerosis:0:4883:0:2:1.00:#NUM!:0.00:#NUM!), (Application.site.discolouration:0:4883:0:2:1.00:#NUM!:0.00:#NUM!), (Application.site.hypoae sthesia:0:4883:0:2:1.00:#NUM!:0.00:#NUM!), (Arterial.aneurysm.repair:0:4883:0:2:1.00:#NUM: 0.00:#NUM!), (Arteriovenous.fistula.occlusion:0:4883:0:2:1.00:#NUM!:0.00:#NUM!), (Asphyx iating.thoracic.dystrophy:0:4883:0:2:1.00:#NUM!:0.00:#NUM!), (Bacteroides.test.positive:0:488 3:0:2:1.00:#NUM!:0.00:#NUM!), (Belligerence:0:4883:0:2:1.00:#NUM!:0.00:#NUM!), (Bence.J ones.protein.urine:0:4883:0:2:1.00:#NUM!:0.00:#NUM!), (Benign.endocrine.neoplasm:0:4883:0: 2:1.00:#NUM!:0.00:#NUM!), (Benign.oesophageal.neoplasm:0:4883:0:2:1.00:#NUM!:0.00:#NU M!), (Biotinidase.deficiency:0:4883:0:2:1.00:#NUM!:0.00:#NUM!), (Birt-Hogg-Dube.syndrome:0:4883:0:2:1.00:#NUM!:0.00:#NUM!), (Birth.mark:0:4883:0:2:1.00:#NUM!:0.0 0:#NUM!), (Bladder.catheter.removal:0:4883:0:2:1.00:#NUM!:0.00:#NUM!), (Bladder. stenosis:0: 4883:0:2:1.00:#NUM!:0.00:#NUM!), (Blepharochalasis:0:4883:0:2:1.00:#NUM!:0.00:#NUM!), (Blood.HIV.RNA.increased:0:4883:0:2:1.00:#NUM!:0.00:#NUM!), (Blood.pressure.immeasurab le:0:4883:0:2:1.00:#NUM!:0.00:#NUM!), (Bone.marrow.donation:0:4883:0:2:1.00:#NUM!:0.00: #NUM!), (Brachioradial.pruritus:0:4883:0:2:1.00:#NUM!:0.00:#NUM!), (Brain.mass:0:4883:0:2: 1.00:#NUM!:0.00:#NUM!), (Breast.injury:0:4883:0:2:1.00:#NUM!:0.00:#NUM!), (Breath.sounds: 0:4883:0:2:1.00:#NUM!:0.00:#NUM!), (Broncholithiasis:0:4883:0:2:1.00:#NUM!:0.00:#NUM!), (Burkholderia.test.positive:0:4883:0:2:1.00:#NUM!:0.00:#NUM!), (Bum.of.internal.organs:0:48 83:0:2:1.00:#NUM!:0.00:#NUM!), (Bums.fourth.degree:0:4883:0:2:1.00:#NUM!:0.00:#NUM!), (Canavan.disease:0:4883:0:2:1.00:#NUM!:0.00:#NUM!), (Carbohydrate.antigen.125. increased:0: 4883:0:2:1.00:#NUM!:0.00:#NUM!), (Carcinoid.crisis:0:4883:0:2:1.00:#NUM!:0.00:#NUM!), (C arcinoma.ex-pleomorphic.adenoma:0:4883:0:2:1.00:#NUM!:0.00:#NUM!), (Carcinoma.in.situ.of.penis:0:488 3:0:2:1.00:#NUM!:0.00:#NUM!), (Cardiac.valve.sclerosis:0:4883:0:2:1.00:#NUM!:0.00:#NUM!), (Cardioactive.drug.level.increased:0:4883:0:2:1.00:#NUM!:0.00:#NUM!), (Cartilage.developm ent.disorder:0:4883:0:2:1.00:#NUM!:0.00:#NUM!), (Cataract.congenital:0:4883:0:2:1.00:#NUM!: 0.00:#NUM!), (Catheter.site.granuloma:0:4883:0:2:1.00:#NUM!:0.00:#NUM!), (CD4.lymphocy tes:0:4883:0:2:1.00:#NUM!:0.00:#NUM!), (Cephalhaematoma:0:4883:0:2:1.00:#NUM!:0.00:#N UM!), (Cerebellar.tumour:0:4883:0:2:1.00:#NUM!:0.00:#NUM!), (Cerebral.vascular.occlusion:0: 4883:0:2:1.00:#NUM!:0.00:#NUM!), (Cervicogenic.vertigo:0:4883:0:2:1.00:#NUM!:0.00:#NU M!), (Cervix.cerclage.procedure:0:4883:0:2:1.00:#NUM!:0.00:#NUM!), (Chemical.eye.injury:0:4 883:0:2:1.00:#NUM!:0.00:#NUM!), (Chemotherapeutic.drug.level.therapeutic:0:4883:0:2:1.00:#NUM!:0.00:#NUM!), (Childhood.depression:0:4883:0:2:1.00:#NUM!:0.00:#NUM!), (Chronic.ga strointestinal.bleeding:0:4883:0:2:1.00:#NUM!:0.00:#NUM!), (Clonal.evolution:0:4883:0:2:1.00: #NUM!:0.00:#NUM!), (Clotting.factor.transfusion:0:4883:0:2:1.00:#NUM!:0.00:#NUM!), (Com puterised.tomogram.kidney.normal:0:4883:0:2:1.00:#NUM!:0.00:#NUM!), (Congenital.central.h ypoventilation.syndrome:0:4883:0:2:1.00:#NUM!:0.00:#NUM!), (Congenital.gastrointestinal.ves sel.anomaly:0:4883:0:2:1.00:#NUM!:0.00:#NUM!), (Congenital.varicella.infection:0:4883:0:2:1. 00:#NUM!:0.00:#NUM!), (Comeal.irritation:0:4883:0:2:1.00:#NUM!:0.00:#NUM!), (Comeal.operation: 0:4883:0:2:1.00:#NUM!:0.00:#NUM!), (Cortisol.free.urine:0:4883:0:2:1.00:#NUM!:0.00: #NUM!), (Cranial.nerve.neoplasm.benign:0:4883:0:2:1.00:#NUM!:0.00:#NUM!), (Craniosynostosis: 0:4883:0:2:1.00:#NUM!:0.00:#NUM!), (Cronobacter.infection:0:4883:0:2:1.00:#NUM!:0.00: #NUM!), (Cryptococcal.fungaemia:0:4883:0:2:1.00:#NUM!:0.00:#NUM!), (Cryptogenic.cirrhosis: 0:4883:0:2:1.00:#NUM!:0.00:#NUM!), (Cryptorchism:0:4883:0:2:1.00:#NUM!:0.00:#NUM!), (CYP2D6.polymorphism:0:4883:0:2:1.00:#NUM!:0.00:#NUM!), (Cystic.eyeball,.congenital:0:48 83:0:2:1.00:#NUM!:0.00:#NUM!), (Cystogram:0:4883:0:2:1.00:#NUM!:0.00:#NUM!), (Cytomeg alovirus.syndrome:0:4883:0:2:1.00:#NUM!:0.00:#NUM!), (Delayed.follicular.ripening:0:4883:0: 2:1.00:#NUM!:0.00:#NUM!), (Delusion.of.replacement:0:4883:0:2:1.00:#NUM!:0.00:#NUM!), (Delusional.disorder,. erotomanic.type:0:4883:0:2:1.00:#NUM!:0.00:#NUM!), (Dental.cyst:0:4883: 0:2:1.00:#NUM!:0.00:#NUM!), (Dental.prosthesis.placement:0:4883:0:2:1.00:#NUM!:0.00:#N UM!), (Desmoplastic.mesothelioma:0:4883:0:2:1.00:#NUM!:0.00:#NUM!), (Developmental.coor dination.disorder:0:4883:0:2:1.00:#NUM!:0.00:#NUM!), (Device.inappropriate.shock.delivery:0: 4883:0:2:1.00:#NUM!:0.00:#NUM!), (Dextrocardia:0:4883:0:2:1.00:#NUM!:0.00:#NUM!), (Dia betic.dermopathy:0:4883:0:2:1.00:#NUM!:0.00:#NUM!), (Diffuse.axonal.injury:0:4883:0:2:1.00: #NUM!:0.00:#NUM!), (Diphyllobothriasis:0:4883:0:2:1.00:#NUM!:0.00:#NUM!), (Disorganised.speech:0:4883:0:2:1.00:#NUM!:0.00:#NUM!), (Drain.removal:0:4883:0:2:1.00:#NUM!:0.00:#N UM!), (Dyscalculia:0:4883:0:2:1.00:#NUM!:0.00:#NUM!), (Dyslalia:0:4883:0:2:1.00:#NUM!:0. 00:#NUM!), (Dyspraxia:0:4883:0:2:1.00:#NUM!:0.00:#NUM!), (Endocarditis.Q.fever:0:4883:0:2: 1.00:#NUM!:0.00:#NUM!), (Endometrial.hypoplasia:0:4883:0:2:1.00:#NUM!:0.00:#NUM!), (En dometrial. sarcoma.recurrent:0:4883:0:2:1.00:#NUM!:0.00:#NUM!), (Entropion:0:4883:0:2:1.00: #NUM!:0.00:#NUM!), (Eosinophil.count.decreased:0:4883:0:2:1.00:#NUM!:0.00:#NUM!), (Epid ermal.growth.factor.receptor.increased:0:4883:0:2:1.00:#NUM!:0.00:#NUM!), (Erythema.dyschr omicum.perstans:0:4883:0:2:1.00:#NUM!:0.00:#NUM!), (Escharotomy:0:4883:0:2:1.00:#NUM!: 0.00:#NUM!), (Excessive.dynamic.airway.collapse:0:4883:0:2:1.00:#NUM!:0.00:#NUM!), (Exhi bitionism:0:4883:0:2:1.00:#NUM!:0.00:#NUM!), (Exposure.to.lead:0:4883:0:2:1.00:#NUM!:0.0 0:#NUM!), (Extemal.ear.disorder:0:4883:0:2:1.00:#NUM!:0.00:#NUM!), (Extemal.ear.pain:0:48 83:0:2:1.00:#NUM!:0.00:#NUM!), (Extradural.haematoma:0:4883:0:2:1.00:#NUM!:0.00:#NUM!), (Extraskeletal.osteosarcoma:0:4883:0:2:1.00:#NUM!:0.00:#NUM!), (Eyelid.cyst:0:4883:0:2:1. 00:#NUM!:0.00:#NUM!), (Eyelid.haemangioma:0:4883:0:2:1.00:#NUM!:0.00:#NUM!), (Eyelid. myoclonus:0:4883:0:2:1.00:#NUM!:0.00:#NUM!), (Factitious.disorder:0:4883:0:2:1.00:#NUM!: 0.00:#NUM!), (Fasciotomy:0:4883:0:2:1.00:#NUM!:0.00:#NUM!), (Fat.redistribution:0:4883:0:2: 1.00:#NUM!:0.00:#NUM!), (Feeling.guilty:0:4883:0:2:1.00:#NUM!:0.00:#NUM!), (Fetishism:0: 4883:0:2:1.00:#NUM!:0.00:#NUM!), (Fibrodysplasia.ossificans.progressiva:0:4883:0:2:1.00:#N UM!:0.00:#NUM!), (Fibrosarcoma.metastatic:0:4883:0:2:1.00:#NUM!:0.00:#NUM!), (Fight.in.school: 0:4883:0:2:1.00:#NUM!:0.00:#NUM!), (Fistulotomy:0:4883:0:2:1.00:#NUM!:0.00:#NUM!), (Follicle.centre.lymphoma.diffuse.small.cell.lymphoma.recurrent:0:4883:0:2:1.00:#NUM!:0.00: #NUM!), (Forced.expiratory.volume.abnormal:0:4883:0:2:1.00:#NUM!:0.00:#NUM!), (Fragile.X.carrier:0:4883:0:2:1.00:#NUM!:0.00:#NUM!), (Galactostasis:0:4883:0:2:1.00:#NUM!:0.00:#NU M!), (Gastric.sarcoma:0:4883:0:2:1.00:#NUM!:0.00:#NUM!), (Gastritis.alcoholic:0:4883:0:2:1.0 0:#NUM!:0.00:#NUM!), (Gastroenteritis.pseudomonas:0:4883:0:2:1.00:#NUM!:0.00:#NUM!), (Gastrointestinal.carcinoma.in.situ:0:4883:0:2:1.00:#NUM!:0.00:#NUM!), (Gastroptosis:0:4883:0: 2:1.00:#NUM!:0.00:#NUM!), (Gender.reassignment.therapy:0:4883:0:2:1.00:#NUM!:0.00:#NU M!), (Genitourinary.chlamydia.infection:0:4883:0:2:1.00:#NUM!:0.00:#NUM!), (Gingivitis.ulcer ative:0:4883:0:2:1.00:#NUM!:0.00:#NUM!), (Glaucoma.traumatic:0:4883:0:2:1.00:#NUM!:0.00: #NUM!), (Grunting:0:4883:0:2:1.00:#NUM!:0.00:#NUM!), (Haemangioma.congenital:0:4883:0: 2:1.00:#NUM!:0.00:#NUM!), (Haemorrhagic.hepatic.cyst:0:4883:0:2:1.00:#NUM!:0.00:#NUM!), (Hairy.cell.leukaemia.recurrent:0:4883:0:2:1.00:#NUM!:0.00:#NUM!), (Hepaplastin.decreased: 0:4883:0:2:1.00:#NUM!:0.00:#NUM!), (Hepatitis.immunisation:0:4883:0:2:1.00:#NUM!:0.00:#NUM!), (Hermansky-Pudlak. syndrome:0:4883:0:2:1.00:#NUM!:0.00:#NUM!), (Hemia.hiatus.repair:0:4883:0:2:1.00:#NUM!:0.00:#NUM!), (Herpes.simplex.hepatitis:0:4883:0:2:1.00:#NUM!:0.00:#NUM!), (Herpes.s implex.meningomyelitis:0:4883:0:2:1.00:#NUM!:0.00:#NUM!), (Histamine.level.decreased:0:48 83:0:2:1.00:#NUM!:0.00:#NUM!), (HIV.infection.CDC.category.A3:0:4883:0:2:1.00:#NUM!:0. 00:#NUM!), (HIV.infection.CDC.group.IV:0:4883:0:2:1.00:#NUM!:0.00:#NUM!), (Hollow.visce ral.myopathy:0:4883:0:2:1.00:#NUM!:0.00:#NUM!), (HTLV-1.test.positive:0:4883:0:2:1.00:#NUM!:0.00:#NUM!), (Human.bite:0:4883:0:2:1.00:#NUM!:0.00: #NUM!), (Hypercholia:0:4883:0:2:1.00:#NUM!:0.00:#NUM!), (Hypersplenism.congenital:0:488 3:0:2:1.00:#NUM!:0.00:#NUM!), (Hyposideraemia:0:4883:0:2:1.00:#NUM!:0.00:#NUM!), (Hyp oxanthine-guanine.phosphoribosyl.transferase.deficiency:0:4883:0:2:1.00:#NUM!:0.00:#NUM!), (Immunog lobulins.increased:0:4883:0:2:1.00:#NUM!:0.00:#NUM!), (Impaired.self-care:0:4883:0:2:1.00:#NUM!:0.00:#NUM!), (Incorrect.route.of.drug.administration:0:4883:0:2:1. 00:#NUM!:0.00:#NUM!), (Infective.thrombosis:0:4883:0:2:1.00:#NUM!:0.00:#NUM!), (Injectio n.site.joint.pain:0:4883:0:2:1.00:#NUM!:0.00:#NUM!), (Insulin.autoimmune.syndrome:0:4883:0:2:1.00:#NUM!:0.00:#NUM!), (Interferon.alpha.level.increased:0:4883:0:2:1.00:#NUM!:0.00:#N UM!), (Interferon.gamma.release.assay.positive:0:4883:0:2:1.00:#NUM!:0.00:#NUM!), (Intestinal. angioedema:0:4883:0:2:1.00:#NUM!:0.00:#NUM!), (Intestinal.malrotation.repair:0:4883:0:2:1. 00:#NUM!:0.00:#NUM!), (Intestinal.transit.time.decreased:0:4883:0:2:1.00:#NUM!:0.00:#NUM!), (Intra-abdominal.fluid.collection:0:4883:0:2:1.00:#NUM!:0.00:#NUM!), (Intra-abdominal.haemangioma:0:4883:0:2:1.00:#NUM!:0.00:#NUM!), (Irlen.syndrome:0:4883:0:2:1.0 0:#NUM!:0.00:#NUM!), (IVth.nerve.disorder:0:4883:0:2:1.00:#NUM!:0.00:#NUM!), (Labour.co mplication:0:4883:0:2:1.00:#NUM!:0.00:#NUM!), (Large.intestinal.stenosis:0:4883:0:2:1.00:#N UM!:0.00:#NUM!), (Laryngeal.operation:0:4883:0:2:1.00:#NUM!:0.00:#NUM!), (Laryngitis.fun gal:0:4883:0:2:1.00:#NUM!:0.00:#NUM!), (Lassa.fever:0:4883:0:2:1.00:#NUM!:0.00:#NUM!), (Latent.syphilis:0:4883:0:2:1.00:#NUM!:0.00:#NUM!), (Lens.dislocation:0:4883:0:2:1.00:#NUM: 0.00:#NUM!), (Lip.and/or.oral.cavity.cancer.stage.II:0:4883:0:2:1.00:#NUM!:0.00:#NUM!), (L ower.extremity.mass:0:4883:0:2:1.00:#NUM!:0.00:#NUM!), (Lymph.node.palpable:0:4883:0:2:1 0.00:#NUM!:0.00:#NUM!), (Lymphoplasmacytoid.lymphoma/immunocytoma.stage.IV:0:4883:0: 2:1.00:#NUM!:0.00:#NUM!), (Macroglossia:0:4883:0:2:1.00:#NUM!:0.00:#NUM!), (Male.orgas mic.disorder:0:4883:0:2:1.00:#NUM!:0.00:#NUM!), (Malignant.neoplasm.of.lacrimal.gland:0:48 83:0:2:1.00:#NUM!:0.00:#NUM!), (Malignant.neoplasm.of.paraurethral.glands:0:4883:0:2:1.00: #NUM!:0.00:#NUM!), (Malignant. ovarian. cyst:0:4883:0:2:1.00:#NUM!:0.00:#NUM!), (Mammo gram.abnormal:0:4883:0:2:1.00:#NUM!:0.00:#NUM!), (Matemal.drugs.affecting.foetus:0:4883:0: 2:1.00:#NUM!:0.00:#NUM!), (Medical.device.site.anaesthesia:0:4883:0:2:1.00:#NUM!:0.00:#N UM!), (Medical.device.site.injury:0:4883:0:2:1.00:#NUM!:0.00:#NUM!), (Melanosis:0:4883:0:2: 1.00:#NUM!:0.00:#NUM!), (Meningitis.aspergillus:0:4883:0:2:1.00:#NUM!:0.00:#NUM!), (Men ingomyelocele:0:4883:0:2:1.00:#NUM!:0.00:#NUM!), (Menopause.delayed:0:4883:0:2:1.00:#N UM!:0.00:#NUM!), (Mesenteric.artery.stent.insertion:0:4883:0:2:1.00:#NUM!:0.00:#NUM!), (M etastatic.glucagonoma:0:4883:0:2:1.00:#NUM!:0.00:#NUM!), (Minimal.residual.disease:0:4883: 0:2:1.00:#NUM!:0.00:#NUM!), (Mitral.commissurotomy:0:4883:0:2:1.00:#NUM!:0.00:#NUM!), (Moraxella.test.positive:0:4883:0:2:1.00:#NUM!:0.00:#NUM!), (Mucinous.cystadenocarcinoma. of.pancreas:0:4883:0:2:1.00:#NUM!:0.00:#NUM!), (Muscle.relaxant.drug.level:0:4883:0:2:1.00: #NUM!:0.00:#NUM!), (Mycotic.comeal.ulcer:0:4883:0:2:1.00:#NUM!:0.00:#NUM!), (Myiasis:0: 4883:0:2:1.00:#NUM!:0.00:#NUM!), (Myositis.ossificans:0:4883:0:2:1.00:#NUM!:0.00:#NUM!), (Nail.bed.bleeding:0:4883:0:2:1.00:#NUM!:0.00:#NUM!), (Nail.bed.inflammation:0:4883:0:2:1 0.00:#NUM!:0.00:#NUM!), (Nasal.mucosal.hypertrophy:0:4883:0:2:1.00:#NUM!:0.00:#NUM!), (Nasal.neoplasm:0:4883:0:2:1.00:#NUM!:0.00:#NUM!), (Necrotising.soft.tissue.infection:0:4883: 0:2:1.00:#NUM!:0.00:#NUM!), (Neonatal.behavioural.syndrome:0:4883:0:2:1.00:#NUM!:0.00:#NUM!), (Nephroangiosclerosis:0:4883:0:2:1.00:#NUM!:0.00:#NUM!), (Neurolysis:0:4883:0:2:1. 00:#NUM!:0.00:#NUM!), (Non-cirrhotic.portal.hypertension:0:4883:0:2:1.00:#NUM!:0.00:#NUM!), (Non-Hodgkin's.lymphoma.unspecified.histology.indolent.stage.IV:0:4883:0:2:1.00:#NUM!:0.00:#NU M!), (Noninfective.myringitis:0:4883:0:2:1.00:#NUM!:0.00:#NUM!), (Nuclear.magnetic.resonan ce.imaging.normal:0:4883:0:2:1.00:#NUM!:0.00:#NUM!), (Occupational.exposure.to.air.contami nants:0:4883:0:2:1.00:#NUM!:0.00:#NUM!), (Oesophageal.adenocarcinoma.recurrent:0:4883:0: 2:1.00:#NUM!:0.00:#NUM!), (Oesophageal.mucosal.tear:0:4883:0:2:1.00:#NUM!:0.00:#NUM!), (Oesophageal.squamous.cell.carcinoma.recurrent:0:4883:0:2:1.00:#NUM!:0.00:#NUM!), (Oral.c avity.neoplasm.surgery:0:4883:0:2:1.00:#NUM!:0.00:#NUM!), (Oral.submucosal.fibrosis:0:4883: 0:2:1.00:#NUM!:0.00:#NUM!), (Organ.failure:0:4883:0:2:1.00:#NUM!:0.00:#NUM!), (Orophar yngeal.blistering:0:4883:0:2:1.00:#NUM!:0.00:#NUM!), (Oropharyngeal.cancer.stage.I:0:4883:0: 2:1.00:#NUM!:0.00:#NUM!), (Oropharyngeal.gonococcal.infection:0:4883:0:2:1.00:#NUM!:0.0 0:#NUM!), (Osteochondritis:0:4883:0:2:1.00:#NUM!:0.00:#NUM!), (Pancreatic.calcification:0:4 883:0:2:1.00:#NUM!:0.00:#NUM!), (Pancreatic.duct.obstruction:0:4883:0:2:1.00:#NUM!:0.00:#NUM!), (Parathyroid.gland.abscess:0:4883:0:2:1.00:#NUM!:0.00:#NUM!), (Paratyphoid.fever:0: 4883:0:2:1.00:#NUM!:0.00:#NUM!), (Parotid.gland.enlargement:0:4883:0:2:1.00:#NUM!:0.00:#NUM!), (Penile.buming.sensation:0:4883:0:2:1.00:#NUM!:0.00:#NUM!), (Penile.curvature:0:488 3:0:2:1.00:#NUM!:0.00:#NUM!), (Perineal.abscess:0:4883:0:2:1.00:#NUM!:0.00:#NUM!), (Peri neal.rash:0:4883:0:2:1.00:#NUM!:0.00:#NUM!), (Periodontal.operation:0:4883:0:2:1.00:#NUM!: 0.00:#NUM!), (Peritoneal.fluid.analysis.normal:0:4883:0:2:1.00:#NUM!:0.00:#NUM!), (Peritone al.haematoma:0:4883:0:2:1.00:#NUM!:0.00:#NUM!), (Peritonitis.pneumococcal:0:4883:0:2:1.00: #NUM!:0.00:#NUM!), (Periventricular.leukomalacia:0:4883:0:2:1.00:#NUM!:0.00:#NUM!), (Pe riventricular.nodular.heterotopia:0:4883:0:2:1.00:#NUM!:0.00:#NUM!), (Phaeochromocytoma.cr isis:0:4883:0:2:1.00:#NUM!:0.00:#NUM!), (Phobia.of.exams:0:4883:0:2:1.00:#NUM!:0.00:#NU M!), (Phytosterolaemia:0:4883:0:2:1.00:#NUM!:0.00:#NUM!), (Placental.dysplasia:0:4883:0:2:1. 00:#NUM!:0.00:#NUM!), (Platelet.function.test:0:4883:0:2:1.00:#NUM!:0.00:#NUM!), (Pleuroth otonus:0:4883:0:2:1.00:#NUM!:0.00:#NUM!), (Poor.quality.drug.administered:0:4883:0:2:1.00:#NUM!:0.00:#NUM!), (Portal.vein.embolisation:0:4883:0:2:1.00:#NUM!:0.00:#NUM!), (Portal.ve in.flow.decreased:0:4883:0:2:1.00:#NUM!:0.00:#NUM!), (Post-injection.delirium.sedation.syndrome:0:4883:0:2:1.00:#NUM!:0.00:#NUM!), (Postpericardiotom y.syndrome:0:4883:0:2:1.00:#NUM!:0.00:#NUM!), (Precerebral.artery.occlusion:0:4883:0:2:1.0 0:#NUM!:0.00:#NUM!), (Product.label.issue:0:4883:0:2:1.00:#NUM!:0.00:#NUM!), (Prolonged. expiration:0:4883:0:2:1.00:#NUM!:0.00:#NUM!), (Prolonged.labour:0:4883:0:2:1.00:#NUM!:0. 00:#NUM!), (Pulmonary.arterial.wedge.pressure.decreased:0:4883:0:2:1.00:#NUM!:0.00:#NUM!), (Pulmonary.contusion:0:4883:0:2:1.00:#NUM!:0.00:#NUM!), (Pulmonary.hypertensive.crisis: 0:4883:0:2:1.00:#NUM!:0.00:#NUM!), (Pupillary.reflex.impaired:0:4883:0:2:1.00:#NUM!:0.00: #NUM!), (Purple.glove.syndrome:0:4883:0:2:1.00:#NUM!:0.00:#NUM!), (Pyromania:0:4883:0:2: 1.00:#NUM!:0.00:#NUM!), (Reaction.to.excipient:0:4883:0:2:1.00:#NUM!:0.00:#NUM!), (Rect al.cancer.stage.0:0:4883:0:2:1.00:#NUM!:0.00:#NUM!), (Renal.aneurysm:0:4883:0:2:1.00:#NU M!:0.00:#NUM!), (Renal.haemangioma:0:4883:0:2:1.00:#NUM!:0.00:#NUM!), (Renal.haemato ma:0:4883:0:2:1.00:#NUM!:0.00:#NUM!), (Renin.abnormal:0:4883:0:2:1.00:#NUM!:0.00:#NU M!), (Renin.decreased:0:4883:0:2:1.00:#NUM!:0.00:#NUM!), (Respiratory.syncytial.virus.bronc hitis:0:4883:0:2:1.00:#NUM!:0.00:#NUM!), (Right.ventricular.outlet.tract.patch:0:4883:0:2:1.00: #NUM!:0.00:#NUM!), (Saccadic.eye.movement:0:4883:0:2:1.00:#NUM!:0.00:#NUM!), (Salivar y.duct.stenosis:0:4883:0:2:1.00:#NUM!:0.00:#NUM!), (Salivary.gland.calculus:0:4883:0:2:1.00: #NUM!:0.00:#NUM!), (Sarcoma.of.skin:0:4883:0:2:1.00:#NUM!:0.00:#NUM!), (Scrotal.abscess: 0:4883:0:2:1.00:#NUM!:0.00:#NUM!), (Scrotal.varicose.veins:0:4883:0:2:1.00:#NUM!:0.00:#N UM!), (Similar.reaction.on.previous.exposure.to.drug:0:4883:0:2:1.00:#NUM!:0.00:#NUM!), (Sk in.hypopigmentation:0:4883:0:2:1.00:#NUM!:0.00:#NUM!), (Slit-lamp.examination:0:4883:0:2:1.00:#NUM!:0.00:#NUM!), (Small.intestine.carcinoma.recurrent:0: 4883:0:2:1.00:#NUM!:0.00:#NUM!), (Soft.tissue.necrosis:0:4883:0:2:1.00:#NUM!:0.00:#NUM!), (Somatoform.disorder.gastrointestinal:0:4883:0:2:1.00:#NUM!:0.00:#NUM!), (Speech.disorder. developmental:0:4883:0:2:1.00:#NUM!:0.00:#NUM!), (Sperm.concentration.abnormal:0:4883:0: 2:1.00:#NUM!:0.00:#NUM!), (Sperm.concentration.increased:0:4883:0:2:1.00:#NUM!:0.00:#N UM!), (Spermatozoa.progressive.motility.decreased:0:4883:0:2:1.00:#NUM!:0.00:#NUM!), (Spin al.rod.insertion:0:4883:0:2:1.00:#NUM!:0.00:#NUM!), (Splenic.marginal.zone.lymphoma.stage.I: 0:4883:0:2:1.00:#NUM!:0.00:#NUM!), (Spontaneous.cerebrospinal.fluid.leak.syndrome:0:4883: 0:2:1.00:#NUM!:0.00:#NUM!), (Sputum.culture:0:4883:0:2:1.00:#NUM!:0.00:#NUM!), (Subacut e.hepatic.failure:0:4883:0:2:1.00:#NUM!:0.00:#NUM!), (Superior.corneal.epithelial.arcuate.lesio n:0:4883:0:2:1.00:#NUM!:0.00:#NUM!), (Superior.limbic.keratoconjunctivitis:0:4883:0:2:1.00:#NUM!:0.00:#NUM!), (Supranuclear.palsy:0:4883:0:2:1.00:#NUM!:0.00:#NUM!), (Suture.related.complication:0:4883:0:2:1.00:#NUM!:0.00:#NUM!), (Swallow.study:0:4883:0:2:1.00:#NUM!:0. 00:#NUM!), (Sweat.discolouration:0:4883:0:2:1.00:#NUM!:0.00:#NUM!), (Swollen.tear.duct:0:4 883:0:2:1.00:#NUM!:0.00:#NUM!), (Talipes:0:4883:0:2:1.00:#NUM!:0.00:#NUM!), (Tendon.she ath.lesion.excision:0:4883:0:2:1.00:#NUM!:0.00:#NUM!), (Testicular.abscess:0:4883:0:2:1.00:#NUM!:0.00:#NUM!), (Testicular.atrophy:0:4883:0:2:1.00:#NUM!:0.00:#NUM!), (Testicular.retra ction:0:4883:0:2:1.00:#NUM!:0.00:#NUM!), (Thoracic.haemorrhage:0:4883:0:2:1.00:#NUM!:0. 00:#NUM!), (Thyroid.electron.radiation.therapy:0:4883:0:2:1.00:#NUM!:0.00:#NUM!), (Tongue. carcinoma.stage.II:0:4883:0:2:1.00:#NUM!:0.00:#NUM!), (Tongue.geographic:0:4883:0:2:1.00: #NUM!:0.00:#NUM!), (Tracheobroncheopathia.osteoclastica:0:4883:0:2:1.00:#NUM!:0.00:#NU M!), (Transitional.cell.cancer.of.the.renal.pelvis.and.ureter.recurrent:0:4883:0:2:1.00:#NUM!:0.0 0:#NUM!), (Trisomy.18:0:4883:0:2:1.00:#NUM!:0.00:#NUM!), (Truncus.coeliacus.thrombosis:0: 4883:0:2:1.00:#NUM!:0.00:#NUM!), (Ultrasound. bladder:0:4883:0:2:1.00:#NUM!:0.00:#NUM!), (Ultrasound.Doppler:0:4883:0:2:1.00:#NUM!:0.00:#NUM!), (Unmasking.of.previously.unident ified.disease:0:4883:0:2:1.00:#NUM!:0.00:#NUM!), (Upper.respiratory.tract.irritation:0:4883:0:2: 1.00:#NUM!:0.00:#NUM!), (Uraemic.pruritus:0:4883:0:2:1.00:#NUM!:0.00:#NUM!), (Urethritis.ureaplasmal:0:4883:0:2:1.00:#NUM!:0.00:#NUM!), (Urine.phosphorus.abnormal:0:4883:0:2:1.0 0:#NUM!:0.00:#NUM!), (Uterine.cervical.erosion:0:4883:0:2:1.00:#NUM!:0.00:#NUM!), (Uterin e.tendemess:0:4883:0:2:1.00:#NUM!:0.00:#NUM!), (Vaccination.site.haematoma:0:4883:0:2:1.0 0:#NUM!:0.00:#NUM!), (Vaginal.cancer.stage.IVA:0:4883:0:2:1.00:#NUM!:0.00:#NUM!), (Vas cular.access.placement:0:4883:0:2:1.00:#NUM!:0.00:#NUM!), (Vascular.pseudoaneurysm. ruptur ed:0:4883:0:2:1.00:#NUM!:0.00:#NUM!), (Victim.of.homicide:0:4883:0:2:1.00:#NUM!:0.00:#N UM!), (Voice.therapy:0:4883:0:2:1.00:#NUM!:0.00:#NUM!), (Vulvovaginal.human.papilloma.vi rus.infection:0:4883:0:2:1.00:#NUM!:0.00:#NUM!), (White.blood.cells.urine.positive:0:4883:0:2: 1.00:#NUM!:0.00:#NUM!), (Wrong.technique.in.product.usage.process:0:4883:0:2:1.00:#NUM!: 0.00:#NUM!), (X-linked.lymphoproliferative.syndrome:0:4883:0:2:1.00:#NUM!:0.00:#NUM!), (Yellow.nail. syndr ome:0:4883:0:2:1.00:#NUM!:0.00:#NUM!), (Autoimmune.lymphoproliferative.syndrome:8:4875:0:2:1.00:1.00:0.80:0.89), (Burkitt's.lymphoma.stage.III:8:4875:0:2:1.00:1.00:0.80:0.89), (Rhabdo id.tumour.of.the.kidney:8:4875:0:2:1.00:1.00:0.80:0.89), (Burkitt's.lymphoma.stage.II:5:4878:0:2: 1.00:1.00:0.71:0.83), (Rheumatoid.factor.negative:5:4878:0:2:1.00:1.00:0.71:0.83), (Autoimmun e.myocarditis:4:4879:0:2:1.00:1.00:0.67:0.80), (Choriomeningitis.lymphocytic:4:4879:0:2:1.00:1. 00:0.67:0.80), (Colonic.fistula:4:4879:0:2:1.00:1.00:0.67:0.80), (Mycobacterial.disease.carrier:4:4 879:0:2:1.00:1.00:0.67:0.80), (Ovarian.embryonal.carcinoma:4:4879:0:2:1.00:1.00:0.67:0.80), (Pi neal.germinoma:4:4879:0:2:1.00:1.00:0.67:0.80), (Blau.syndrome:3:4880:0:2:1.00:1.00:0.60:0.75), (Chorioretinal.disorder:3:4880:0:2:1.00:1.00:0.60:0.75), (Chronic.lymphocytic.inflammation.wi th.pontine.perivascular.enhancement.responsive.to.steroids:3:4880:0:2:1.00:1.00:0.60:0.75), (Extr adural.neoplasm:3:4880:0:2:1.00:1.00:0.60:0.75), (Interleukin.level.decreased:3:4880:0:2:1.00:1. 00:0.60:0.75), (Non-Hodgkin's.lymphoma.unspecified.histology.aggressive.recurrent:3:4880:0:2:1.00:1.00:0.60:0.75), (Nuclear.magnetic.resonance.imaging.thoracic:3:4880:0:2:1.00:1.00:0.60:0.75), (Paternal.exposu re.timing.unspecified:3:4880:0:2:1.00:1.00:0.60:0.75), (Selective.mutism:3:4880:0:2:1.00:1.00:0. 60:0.75), (Soft.tissue.mass:3:4880:0:2:1.00:1.00:0.60:0.75), (Abortion.incomplete:2:4881:0:2:1.00: 1.00:0.50:0.67), (Adenosquamous.cell.lung.cancer.stage.III:2:4881:0:2:1.00:1.00:0.50:0.67), (Am phetamines.positive:2:4881:0:2:1.00:1.00:0.50:0.67), (Apallic.syndrome:2:4881:0:2:1.00:1.00:0.5 0:0.67), (Bezoar:2:4881:0:2:1.00:1.00:0.50:0.67), (Blood.folate.increased:2:4881:0:2:1.00:1.00:0. 50:0.67), (Carcinoid.tumour.of.the.prostate:2:4881:0:2:1.00:1.00:0.50:0.67), (Cardiac.valve.fibroe lastoma:2:4881:0:2:1.00:1.00:0.50:0.67), (Cerebellar.dysplasia:2:4881:0:2:1.00:1.00:0.50:0.67), (Cerebellar.mutism:2:4881:0:2:1.00:1.00:0.50:0.67), (Chediak-Higashi.syndrome:2:4881:0:2:1.00:1.00:0.50:0.67), (Chronic.lymphocytic.leukaemia.stage.0:2:48 81:0:2:1.00:1.00:0.50:0.67), (Ileal.ulcer:2:4881:0:2:1.00:1.00:0.50:0.67), (Injection.site.bruising:2: 4881:0:2:1.00:1.00:0.50:0.67), (Ocular.vasculitis:2:4881:0:2:1.00:1.00:0.50:0.67), (Ovarian.germ.cell.teratoma.stage.IV:2:4881:0:2:1.00:1.00:0.50:0.67), (Pericarditis.lupus:2:4881:0:2:1.00:1.00: 0.50:0.67), (Phospholipidosis:2:4881:0:2:1.00:1.00:0.50:0.67), (Polymorphic.light.eruption:2:488 1:0:2:1.00:1.00:0.50:0.67), (Retinal.aneurysm:2:4881:0:2:1.00:1.00:0.50:0.67), (Skin.induration:2: 4881:0:2:1.00:1.00:0.50:0.67), (Testicular.embryonal.carcinoma.stage.I:2:4881:0:2:1.00:1.00:0.5 0:0.67), (Testicular.germ.cell.tumour.mixed.stage.II:2:4881:0:2:1.00:1.00:0.50:0.67), (Testicular.s eminoma.(pure).stage.III:2:4881:0:2:1.00:1.00:0.50:0.67), (Type.III.immune.complex.mediated.r eaction:2:4881:0:2:1.00:1.00:0.50:0.67), (Vaginal.neoplasm:2:4881:0:2:1.00:1.00:0.50:0.67), (Ve ntricular.septal.defect.repair:2:4881:0:2:1.00:1.00:0.50:0.67), (Viral.sinusitis:2:4881:0:2:1.00:1.0 0:0.50:0.67), (Abnormal.product.of.conception:1:4882:0:2:1.00:1.00:0.33:0.50), (Acute.monocyti c.leukaemia.(in.remission):1:4882:0:2:1.00:1.00:0.33:0.50), (Acute.right.ventricular.failure:1:488 2:0:2:1.00:1.00:0.33:0.50), (Application.site.dryness:1:4882:0:2:1.00:1.00:0.33:0.50), (Arterioven ous.fistula.operation:1:4882:0:2:1.00:1.00:0.33:0.50), (Bladder.mass:1:4882:0:2:1.00:1.00:0.33:0. 50), (Bladder.squamous.cell.carcinoma.stage.unspecified:1:4882:0:2:1.00:1.00:0.33:0.50), (Blood. beryllium:1:4882:0:2:1.00:1.00:0.33:0.50), (Blood.luteinising.hormone:1:4882:0:2:1.00:1.00:0.33: 0.50), (Blood.product.transfusion:1:4882:0:2:1.00:1.00:0.33:0.50), (Body.height.abnormal:1:488 2:0:2:1.00:1.00:0.33:0.50), (Body.mass.index.abnormal:1:4882:0:2:1.00:1.00:0.33:0.50), (C-kit.gene.mutation:1:4882:0:2:1.00:1.00:0.33:0.50), (Cholangitis.infective:1:4882:0:2:1.00:1.00:0. 33:0.50), (Chorioretinal.atrophy:1:4882:0:2:1.00:1.00:0.33:0.50), (Congenital.hypercoagulation:1: 4882:0:2:1.00:1.00:0.33:0.50), (Congenital.musculoskeletal.anomaly:1:4882:0:2:1.00:1.00:0.33:0 0.50), (Congenital.spinal.cord.anomaly:1:4882:0:2:1.00:1.00:0.33:0.50), (Corneal.neovascularisatio n:1:4882:0:2:1.00:1.00:0.33:0.50), (Coronary.vascular.graft.occlusion:1:4882:0:2:1.00:1.00:0.33: 0.50), (Cox-Maze.procedure:1:4882:0:2:1.00:1.00:0.33:0.50), (Cystitis.glandularis:1:4882:0:2:1.00:1.00:0.33: 0.50), (Device.dislocation:1:4882:0:2:1.00:1.00:0.33:0.50), (Drug-disease.interaction:1:4882:0:2:1.00:1.00:0.33:0.50), (Drug.specific.antibody.absent:1:4882:0:2:1. 00:1.00:0.33:0.50), (Electrocardiogram.RR.interval.prolonged:1:4882:0:2:1.00:1.00:0.33:0.50), (E lectrocution:1:4882:0:2:1.00:1.00:0.33:0.50), (Encephalitis.allergic:1:4882:0:2:1.00:1.00:0.33:0.5 0), (Endometrial.ablation:1:4882:0:2:1.00:1.00:0.33:0.50), (Endometrial.thickening:1:4882:0:2:1. 00:1.00:0.33:0.50), (Erythroblastosis.foetalis:1:4882:0:2:1.00:1.00:0.33:0.50), (Erythroid.maturati on.arrest:1:4882:0:2:1.00:1.00:0.33:0.50), (Eye.haemangioma:1:4882:0:2:1.00:1.00:0.33:0.50), (F eminisation.acquired:1:4882:0:2:1.00:1.00:0.33:0.50), (Fracture.malunion:1:4882:0:2:1.00:1.00:0 0.33:0.50), (Fungating.wound:1:4882:0:2:1.00:1.00:0.33:0.50), (Haemorrhagic.ovarian.cyst:1:4882: 0:2:1.00:1.00:0.33:0.50), (Hereditary.stomatocytosis:1:4882:0:2:1.00:1.00:0.33:0.50), (Hyperfibri nogenaemia:1:4882:0:2:1.00:1.00:0.33:0.50), (Infertility.tests:1:4882:0:2:1.00:1.00:0.33:0.50), (In fusion.site.induration:1:4882:0:2:1.00:1.00:0.33:0.50), (Injection.site.oedema:1:4882:0:2:1.00:1.0 0:0.33:0.50), (Ischaemic.neuropathy:1:4882:0:2:1.00:1.00:0.33:0.50), (Jaw.operation:1:4882:0:2:1 0.00:1.00:0.33:0.50), (JC.virus.infection:1:4882:0:2:1.00:1.00:0.33:0.50), (Laryngeal.neoplasm:1:4 882:0:2:1.00:1.00:0.33:0.50), (Lymph.node.abscess:1:4882:0:2:1.00:1.00:0.33:0.50), (Mean.cell.v olume:1:4882:0:2:1.00:1.00:0.33:0.50), (Meconium.ileus:1:4882:0:2:1.00:1.00:0.33:0.50), (Mesen teric.artery.stenosis:1:4882:0:2:1.00:1.00:0.33:0.50), (Metastases.to.reproductive.organ:1:4882:0: 2:1.00:1.00:0.33:0.50), (Microcytosis:1:4882:0:2:1.00:1.00:0.33:0.50), (Mitochondrial.DNA.muta tion:1:4882:0:2:1.00:1.00:0.33:0.50), (Muscle.enzyme.increased:1:4882:0:2:1.00:1.00:0.33:0.50), (Myasthenia.gravis.neonatal:1:4882:0:2:1.00:1.00:0.33:0.50), (Nail.pigmentation:1:4882:0:2:1.00: 1.00:0.33:0.50), (Nasal.turbinate.hypertrophy:1:4882:0:2:1.00:1.00:0.33:0.50), (Natural.killer.cell.count.decreased:1:4882:0:2:1.00:1.00:0.33:0.50), (Ovarian.granulosa-theca.cell.tumour:1:4882:0:2:1.00:1.00:0.33:0.50), (Palisaded.neutrophilic.granulomatous.dermat itis:1:4882:0:2:1.00:1.00:0.33:0.50), (Penis.injury:1:4882:0:2:1.00:1.00:0.33:0.50), (Personality.di sorder.of.childhood:1:4882:0:2:1.00:1.00:0.33:0.50), (Polymicrogyria:1:4882:0:2:1.00:1.00:0.33: 0.50), (Protein.S:1:4882:0:2:1.00:1.00:0.33:0.50), (Pterygium.operation:1:4882:0:2:1.00:1.00:0.33: 0.50), (Pulmonary.artery.dilatation:1:4882:0:2:1.00:1.00:0.33:0.50), (Pulmonary.lymphangiectasi a:1:4882:0:2:1.00:1.00:0.33:0.50), (Pulmonary.venous.thrombosis:1:4882:0:2:1.00:1.00:0.33:0.50), (Rectosigmoid.cancer.stage.I:1:4882:0:2:1.00:1.00:0.33:0.50), (Renal.cell.carcinoma.stage.II:1: 4882:0:2:1.00:1.00:0.33:0.50), (Rhinophyma:1:4882:0:2:1.00:1.00:0.33:0.50), (Scleral.hyperaemi a:1:4882:0:2:1.00:1.00:0.33:0.50), (Selective.abortion:1:4882:0:2:1.00:1.00:0.33:0.50), (Sensory.g anglionitis:1:4882:0:2:1.00:1.00:0.33:0.50), (Seroma:1:4882:0:2:1.00:1.00:0.33:0.50), (Subclavia n.vein.stenosis:1:4882:0:2:1.00:1.00:0.33:0.50), (Superficial.spreading.melanoma.stage.IV:1:488 2:0:2:1.00:1.00:0.33:0.50), (T-cell.lymphoma.stage.III:1:4882:0:2:1.00:1.00:0.33:0.50), (Tenoplasty:1:4882:0:2:1.00:1.00:0.33: 0.50), (Tongue.oedema:1:4882:0:2:1.00:1.00:0.33:0.50), (Traumatic.liver.injury:1:4882:0:2:1.00:1 0.00:0.33:0.50), (Uterine.compression.sutures:1:4882:0:2:1.00:1.00:0.33:0.50), (Uterovaginal.prola pse:1:4882:0:2:1.00:1.00:0.33:0.50), (Vascular.rupture:1:4882:0:2:1.00:1.00:0.33:0.50), (Vitreous.disorder:1:4882:0:2:1.00:1.00:0.33:0.50), (Acetonaemia:0:4882:0:3:1.00:#NUM!:0.00:#NUM!), (Acid.base.balance.normal:0:4882:0:3:1.00:#NUM!:0.00:#NUM!), (Acrodermatitis.chronica.atrop hicans:0:4882:0:3:1.00:#NUM!:0.00:#NUM!), (Actinomyces.test.positive:0:4882:0:3:1.00:#NU M!:0.00:#NUM!), (Actinomycotic.skin.infection:0:4882:0:3:1.00:#NUM!:0.00:#NUM!), (Adnexa.uteri.mass:0:4882:0:3:1.00:#NUM!:0.00:#NUM!), (Air.embolism:0:4882:0:3:1.00:#NUM!:0.00: #NUM!), (Alanine.aminotransferase.abnormal:0:4882:0:3:1.00:#NUM!:0.00:#NUM!), (Ammonia.normal:0:4882:0:3:1.00:#NUM!:0.00:#NUM!), (Amniorrhexis:0:4882:0:3:1.00:#NUM!:0.00:#N UM!), (Analgesic.drug.level.increased:0:4882:0:3:1.00:#NUM!:0.00:#NUM!), (Aortitis.salmonell a:0:4882:0:3:1.00:#NUM!:0.00:#NUM!), (Apolipoprotein.increased:0:4882:0:3:1.00:#NUM!:0.0 0:#NUM!), (Application.site.bum:0:4882:0:3:1.00:#NUM!:0.00:#NUM!), (Application.site.vesicl es:0:4882:0:3:1.00:#NUM!:0.00:#NUM!), (Arteriovenous.graft.site.infection:0:4882:0:3:1.00:#N UM!:0.00:#NUM!), (Arteriovenous.graft.thrombosis:0:4882:0:3:1.00:#NUM!:0.00:#NUM!), (Arti ficial.insemination.by.donor:0:4882:0:3:1.00:#NUM!:0.00:#NUM!), (Atrial.pressure.increased:0: 4882:0:3:1.00:#NUM!:0.00:#NUM!), (Attention-seeking.behaviour:0:4882:0:3:1.00:#NUM!:0.00:#NUM!), (Atypical.mycobacterial.lower.respirat ory.tract.infection:0:4882:0:3:1.00:#NUM!:0.00:#NUM!), (Automatism:0:4882:0:3:1.00:#NUM!: 0.00:#NUM!), (Balneotherapy:0:4882:0:3:1.00:#NUM!:0.00:#NUM!), (Basilar.artery.aneurysm:0: 4882:0:3:1.00:#NUM!:0.00:#NUM!), (Bence.Jones.protein.urine.present:0:4882:0:3:1.00:#NUM!: 0.00:#NUM!), (Benign.gastric.neoplasm:0:4882:0:3:1.00:#NUM!:0.00:#NUM!), (Beta.interfero n.therapy:0:4882:0:3:1.00:#NUM!:0.00:#NUM!), (Biopsy.bile.duct.abnormal:0:4882:0:3:1.00:#N UM!:0.00:#NUM!), (Blast.injury:0:4882:0:3:1.00:#NUM!:0.00:#NUM!), (Bleeding.time.abnorma 1:0:4882:0:3:1.00:#NUM!:0.00:#NUM!), (Bleeding.time.shortened:0:4882:0:3:1.00:#NUM!:0.00: #NUM!), (Bleeding.varicose.vein:0:4882:0:3:1.00:#NUM!:0.00:#NUM!), (Blindness.cortical:0:48 82:0:3:1.00:#NUM!:0.00:#NUM!), (Blood.immunoglobulin.E.abnormal:0:4882:0:3:1.00:#NUM!: 0.00:#NUM!), (Blood.immunoglobulin.G.normal:0:4882:0:3:1.00:#NUM!:0.00:#NUM!), (Brain. stem.thrombosis:0:4882:0:3:1.00:#NUM!:0.00:#NUM!), (Bronchial.wall.thickening:0:4882:0:3:1 0.00:#NUM!:0.00:#NUM!), (Brucella.test.positive:0:4882:0:3:1.00:#NUM!:0.00:#NUM!), (Bulbar. palsy:0:4882:0:3:1.00:#NUM!:0.00:#NUM!), (Buttock.injury:0:4882:0:3:1.00:#NUM!:0.00:#NU M!), (Candida.nappy.rash:0:4882:0:3:1.00:#NUM!:0.00:#NUM!), (Cardiac.perforation:0:4882:0:3 1.00:#NUM!:0.00:#NUM!), (Cardiac.valve.rupture:0:4882:0:3:1.00:#NUM!:0.00:#NUM!), (Caro tid.intima-media.thickness.increased:0:4882:0:3:1.00:#NUM!:0.00:#NUM!), (Catecholamines.urine.increas ed:0:4882:0:3:1.00:#NUM!:0.00:#NUM!), (Catheter.site.haemorrhage:0:4882:0:3:1.00:#NUM!:0 0.00:#NUM!), (Cerebellar.artery.thrombosis:0:4882:0:3:1.00:#NUM!:0.00:#NUM!), (Child.maltre atment.syndrome:0:4882:0:3:1.00:#NUM!:0.00:#NUM!), (Chlamydial.cervicitis:0:4882:0:3:1.00: #NUM!:0.00:#NUM!), (Cholera:0:4882:0:3:1.00:#NUM!:0.00:#NUM!), (Chondrolysis:0:4882:0: 3:1.00:#NUM!:0.00:#NUM!), (Chondrosarcoma.recurrent:0:4882:0:3:1.00:#NUM!:0.00:#NUM!), (Coagulation.factor.IX.level:0:4882:0:3:1.00:#NUM!:0.00:#NUM!), (Coagulation.factor.VIII.lev el.decreased:0:4882:0:3:1.00:#NUM!:0.00:#NUM!), (Coating.in.mouth:0:4882:0:3:1.00:#NUM!: 0.00:#NUM!), (Colonoscopy.normal:0:4882:0:3:1.00:#NUM!:0.00:#NUM!), (Colorectal.carcino ma.stage.0:0:4882:0:3:1.00:#NUM!:0.00:#NUM!), (Compulsive.shopping:0:4882:0:3:1.00:#NU M!:0.00:#NUM!), (Computerised.tomogram.liver.abnormal:0:4882:0:3:1.00:#NUM!:0.00:#NUM!), (Confusional.arousal:0:4882:0:3:1.00:#NUM!:0.00:#NUM!), (Congenital.brain.damage:0:4882: 0:3:1.00:#NUM!:0.00:#NUM!), (Congenital.herpes.simplex.infection:0:4882:0:3:1.00:#NUM!:0 0.00:#NUM!), (Congenital.multiplex.arthrogryposis:0:4882:0:3:1.00:#NUM!:0.00:#NUM!), (Cong enital.nephrogenic.diabetes.insipidus:0:4882:0:3:1.00:#NUM!:0.00:#NUM!), (Conjunctival.irritat ion:0:4882:0:3:1.00:#NUM!:0.00:#NUM!), (Copper.deficiency:0:4882:0:3:1.00:#NUM!:0.00:#N UM!), (Corneal.abscess:0:4882:0:3:1.00:#NUM!:0.00:#NUM!), (Crime:0:4882:0:3:1.00:#NUM!: 0.00:#NUM!), (Cyanosis.central:0:4882:0:3:1.00:#NUM!:0.00:#NUM!), (Cytomegalovirus.myelo meningoradiculitis:0:4882:0:3:1.00:#NUM!:0.00:#NUM!), (Cytomegalovirus.myocarditis:0:4882:0:3:1.00:#NUM!:0.00:#NUM!), (Dacryocanaliculitis:0:4882:0:3:1.00:#NUM!:0.00:#NUM!), (De.Lange's.syndrome:0:4882:0:3:1.00:#NUM!:0.00:#NUM!), (Deafness.bilateral:0:4882:0:3:1.00:#NUM!:0.00:#NUM!), (Defect.conduction.intraventricular:0:4882:0:3:1.00:#NUM!:0.00:#NUM!), (Deficiency.of.the.interleukin-1.receptor.antagonist:0:4882:0:3:1.00:#NUM!:0.00:#NUM!), (Delusion.of.reference:0:4882:0:3:1 00:#NUM!:0.00:#NUM!), (Dementia.of.the.Alzheimer's.type,.with.depressed.mood:0:4882:0:3:1 00:#NUM!:0.00:#NUM!), (Dexamethasone.suppression.test:0:4882:0:3:1.00:#NUM!:0.00:#NU M!), (Drug.delivery.device.implantation:0:4882:0:3:1.00:#NUM!:0.00:#NUM!), (Drug.dependene e,.postpartum:0:4882:0:3:1.00:#NUM!:0.00:#NUM!), (Ear.canal.erythema:0:4882:0:3:1.00:#NU M!:0.00:#NUM!), (Ear.infection.viral:0:4882:0:3:1.00:#NUM!:0.00:#NUM!), (Early.satiety:0:488 2:0:3:1.00:#NUM!:0.00:#NUM!), (Eating.disorder.symptom:0:4882:0:3:1.00:#NUM!:0.00:#NU M!), (Eczema.weeping:0:4882:0:3:1.00:#NUM!:0.00:#NUM!), (Electrocardiogram.change:0:488 2:0:3:1.00:#NUM!:0.00:#NUM!), (Embolic.pneumonia:0:4882:0:3:1.00:#NUM!:0.00:#NUM!), (Emergency.care.examination.abnormal:0:4882:0:3:1.00:#NUM!:0.00:#NUM!), (Epiphyseal.disor der:0:4882:0:3:1.00:#NUM!:0.00:#NUM!), (Eustachian.tube.patulous:0:4882:0:3:1.00:#NUM!:0. 00:#NUM!), (Exposure.via.direct.contact:0:4882:0:3:1.00:#NUM!:0.00:#NUM!), (Extragonadal.p rimary. germ.cell.tumour.mixed.stage.:0:4882:0:3:1.00:#NUM!:0.00:#NUM!), (Eyelash.injury:0: 4882:0:3:1.00:#NUM!:0.00:#NUM!), (Facial.nerve.decompression:0:4882:0:3:1.00:#NUM!:0.00: #NUM!), (Fascial.infection:0:4882:0:3:1.00:#NUM!:0.00:#NUM!), (Foetal.heart.rate.decreased:0: 4882:0:3:1.00:#NUM!:0.00:#NUM!), (Follicle.centre.lymphoma,.follicular.grade.I,.II,.III.refracto ry:0:4882:0:3:1.00:#NUM!:0.00:#NUM!), (Foramen.magnum.stenosis:0:4882:0:3:1.00:#NUM!:0 0.00:#NUM!), (Fractional.flow.reserve:0:4882:0:3:1.00:#NUM!:0.00:#NUM!), (Gastric.cancer.stag e.I:0:4882:0:3:1.00:#NUM!:0.00:#NUM!), (Gastroenteritis.vibrio:0:4882:0:3:1.00:#NUM!:0.00:#NUM!), (Gastrointestinal.amyloidosis:0:4882:0:3:1.00:#NUM!:0.00:#NUM!), (Gastrointestinal.m ucosa.hyperaemia:0:4882:0:3:1.00:#NUM!:0.00:#NUM!), (Gastrointestinal.stoma.output.decreas ed:0:4882:0:3:1.00:#NUM!:0.00:#NUM!), (Gastrointestinal.tract.adenoma:0:4882:0:3:1.00:#NU M!:0.00:#NUM!), (Genital.atrophy:0:4882:0:3:1.00:#NUM!:0.00:#NUM!), (Glycogen.storage. dis ease.type.VI:0:4882:0:3:1.00:#NUM!:0.00:#NUM!), (Haematospermia:0:4882:0:3:1.00:#NUM!: 0.00:#NUM!), (Haemoglobin.E:0:4882:0:3:1.00:#NUM!:0.00:#NUM!), (Haemophilia.A.without. Inhibitors:0:4882:0:3:1.00:#NUM!:0.00:#NUM!), (Halo.vision:0:4882:0:3:1.00:#NUM!:0.00:#N UM!), (Hand.amputation:0:4882:0:3:1.00:#NUM!:0.00:#NUM!), (Head.banging:0:4882:0:3:1.00: #NUM!:0.00:#NUM!), (Hepatic.necrosis:0:4882:0:3:1.00:#NUM!:0.00:#NUM!), (Histology:0:48 82:0:3:1.00:#NUM!:0.00:#NUM!), (HIV.infection.CDC.Group.IV.subgroup.D:0:4882:0:3:1.00:#NUM!:0.00:#NUM!), (Homosexuality:0:4882:0:3:1.00:#NUM!:0.00:#NUM!), (Humidity.intolera nce:0:4882:0:3:1.00:#NUM!:0.00:#NUM!), (Hypergastrinaemia:0:4882:0:3:1.00:#NUM!:0.00:#NUM!), (Hypertensive.angiopathy:0:4882:0:3:1.00:#NUM!:0.00:#NUM!), (Hypocalcaemic.seizur e:0:4882:0:3:1.00:#NUM!:0.00:#NUM!), (Impatience:0:4882:0:3:1.00:#NUM!:0.00:#NUM!), (Im plantable.defibrillator.replacement:0:4882:0:3:1.00:#NUM!:0.00:#NUM!), (Incision.site.inflamm ation:0:4882:0:3:1.00:#NUM!:0.00:#NUM!), (Incorrect.drug.administration.rate:0:4882:0:3:1.00: #NUM!:0.00:#NUM!), (Infantile.colic:0:4882:0:3:1.00:#NUM!:0.00:#NUM!), (Infantile.spitting. up:0:4882:0:3:1.00:#NUM!:0.00:#NUM!), (Infective.tenosynovitis:0:4882:0:3:1.00:#NUM!:0.00: #NUM!), (Intestinal.cyst:0:4882:0:3:1.00:#NUM!:0.00:#NUM!), (Intestinal.prolapse:0:4882:0:3: 1.00:#NUM!:0.00:#NUM!), (Intracranial.mass:0:4882:0:3:1.00:#NUM!:0.00:#NUM!), (Intraductal. papillary-mucinous.carcinoma.of.pancreas:0:4882:0:3:1.00:#NUM!:0.00:#NUM!), (Irritability.postvaccinal: 0:4882:0:3:1.00:#NUM!:0.00:#NUM!), (Japanese.spotted.fever:0:4882:0:3:1.00:#NUM!:0.00:#NUM!), (Job.change:0:4882:0:3:1.00:#NUM!:0.00:#NUM!), (Juvenile.angiofibroma:0:4882:0:3:1 0.00:#NUM!:0.00:#NUM!), (Keratitis-ichthyosis-deafness.syndrome:0:4882:0:3:1.00:#NUM!:0.00:#NUM!), (Laryngeal.cancer.stage.I:0:4882:0:3: 1.00:#NUM!:0.00:#NUM!), (Laryngeal.granuloma:0:4882:0:3:1.00:#NUM!:0.00:#NUM!), (Lary nx.irritation:0:4882:0:3:1.00:#NUM!:0.00:#NUM!), (Lenticular.opacities:0:4882:0:3:1.00:#NUM !:0.00:#NUM!), (Limb.deformity:0:4882:0:3:1.00:#NUM!:0.00:#NUM!), (Linitis.plastica:0:4882: 0:3:1.00:#NUM!:0.00:#NUM!), (Lipiduria:0:4882:0:3:1.00:#NUM!:0.00:#NUM!), (Lymphangiec tasia:0:4882:0:3:1.00:#NUM!:0.00:#NUM!), (Lymphangiosarcoma:0:4882:0:3:1.00:#NUM!:0.00: #NUM!), (Lymphocele.marsupialisation:0:4882:0:3:1.00:#NUM!:0.00:#NUM!), (Lymphocyte.co unt:0:4882:0:3:1.00:#NUM!:0.00:#NUM!), (Malabsorption.from.injection.site:0:4882:0:3:1.00:#NUM!:0.00:#NUM!), (Malignant.haemangiopericytoma:0:4882:0:3:1.00:#NUM!:0.00:#NUM!), (Malignant.neoplasm.papilla.of.Vater:0:4882:0:3:1.00:#NUM!:0.00:#NUM!), (Medication.residue.present:0:4882:0:3:1.00:#NUM!:0.00:#NUM!), (Meningoencephalitis.adenoviral:0:4882:0:3:1.0 0:#NUM!:0.00:#NUM!), (Mental.status.changes.postoperative:0:4882:0:3:1.00:#NUM!:0.00:#N UM!), (Metabolic.encephalopathy:0:4882:0:3:1.00:#NUM!:0.00:#NUM!), (Metastases.to.peripher al.nervous.system:0:4882:0:3:1.00:#NUM!:0.00:#NUM!), (Metastases.to.pharynx:0:4882:0:3:1.0 0:#NUM!:0.00:#NUM!), (Metastases.to.vagina:0:4882:0:3:1.00:#NUM!:0.00:#NUM!), (Micrococ cus.infection:0:4882:0:3:1.00:#NUM!:0.00:#NUM!), (Microgenia:0:4882:0:3:1.00:#NUM!:0.00: #NUM!), (Multiple.lentigines.syndrome:0:4882:0:3:1.00:#NUM!:0.00:#NUM!), (Muscle.necrosis: 0:4882:0:3:1.00:#NUM!:0.00:#NUM!), (Nasal.valve.collapse:0:4882:0:3:1.00:#NUM!:0.00:#N UM!), (Nasopharyngeal.cancer.stage.I:0:4882:0:3:1.00:#NUM!:0.00:#NUM!), (Necrotising.ulcera tive.gingivostomatitis:0:4882:0:3:1.00:#NUM!:0.00:#NUM!), (Neuritis. cranial:0:4882:0:3:1.00:#NUM!:0.00:#NUM!), (Neuropathic.ulcer:0:4882:0:3:1.00:#NUM!:0.00:#NUM!), (New.daily.pers istent.headache:0:4882:0:3:1.00:#NUM!:0.00:#NUM!), (Nocardia.test.positive:0:4882:0:3:1.00:#NUM!:0.00:#NUM!), (Non-Hodgkin's.lymphoma.transformed.recurrent:0:4882:0:3:1.00:#NUM!:0.00:#NUM!), (Oesophageal. squamous.cell.carcinoma.stage.I:0:4882:0:3:1.00:#NUM!:0.00:#NUM!), (Oesophageal.squamo us.cell.carcinoma.stage.II:0:4882:0:3:1.00:#NUM!:0.00:#NUM!), (Oestrogenic.effect:0:4882:0:3: 1.00:#NUM!:0.00:#NUM!), (Olfacto.genital.dysplasia:0:4882:0:3:1.00:#NUM!:0.00:#NUM!), (O ligospermia:0:4882:0:3:1.00:#NUM!:0.00:#NUM!), (Ovarian.abscess:0:4882:0:3:1.00:#NUM!:0. 00:#NUM!), (Ovarian.stromal.cancer:0:4882:0:3:1.00:#NUM!:0.00:#NUM!), (Pancreatic.duct.dil atation:0:4882:0:3:1.00:#NUM!:0.00:#NUM!), (Pancreatic.injury:0:4882:0:3:1.00:#NUM!:0.00:#NUM!), (Papillitis:0:4882:0:3:1.00:#NUM!:0.00:#NUM!), (Parent-child.problem:0:4882:0:3:1.00:#NUM!:0.00:#NUM!), (Patent.ductus.arteriosus.repair:0:4882:0:3 0.1.00:#NUM!:0.00:#NUM!), (Pathological.gambling:0:4882:0:3:1.00:#NUM!:0.00:#NUM!), (Pati ent.isolation:0:4882:0:3:1.00:#NUM!:0.00:#NUM!), (Pelvic.fluid.collection:0:4882:0:3:1.00:#N UM!:0.00:#NUM!), (Penicilliosis:0:4882:0:3:1.00:#NUM!:0.00:#NUM!), (Penile.operation:0:488 2:0:3:1.00:#NUM!:0.00:#NUM!), (Periorbital.haemorrhage:0:4882:0:3:1.00:#NUM!:0.00:#NUM !), (Persistent.generalised.lymphadenopathy:0:4882:0:3:1.00:#NUM!:0.00:#NUM!), (Pharyngeal. paraesthesia:0:4882:0:3:1.00:#NUM!:0.00:#NUM!), (Pineal.neoplasm:0:4882:0:3:1.00:#NUM!:0 0.00:#NUM!), (Pituitary.tumour.recurrent:0:4882:0:3:1.00:#NUM!:0.00:#NUM!), (Plasmin.inhibit or:0:4882:0:3:1.00:#NUM!:0.00:#NUM!), (Pneumonia.parainfluenzae.viral:0:4882:0:3:1.00:#NU M!:0.00:#NUM!), (Post.embolisation.syndrome:0:4882:0:3:1.00:#NUM!:0.00:#NUM!), (Post.pro cedural.haematoma:0:4882:0:3:1.00:#NUM!:0.00:#NUM!), (Postcricoid.cancer:0:4882:0:3:1.00: #NUM!:0.00:#NUM!), (Postoperative.abscess:0:4882:0:3:1.00:#NUM!:0.00:#NUM!), (Postpartu m.uterine.subinvolution:0:4882:0:3:1.00:#NUM!:0.00:#NUM!), (Potassium.wasting.nephropathy: 0:4882:0:3:1.00:#NUM!:0.00:#NUM!), (Progeria:0:4882:0:3:1.00:#NUM!:0.00:#NUM!), (Psych ogenic.erectile.dysfunction:0:4882:0:3:1.00:#NUM!:0.00:#NUM!), (Pulmonary.malformation:0:4 882:0:3:1.00:#NUM!:0.00:#NUM!), (Pulmonary.resection:0:4882:0:3:1.00:#NUM!:0.00:#NUM!), (Pupils.unequal:0:4882:0:3:1.00:#NUM!:0.00:#NUM!), (Pyelocystitis:0:4882:0:3:1.00:#NUM!: 0.00:#NUM!), (Pyelonephritis.fungal:0:4882:0:3:1.00:#NUM!:0.00:#NUM!), (Rebound.psychosis: 0:4882:0:3:1.00:#NUM!:0.00:#NUM!), (Rubella.infection.neurological:0:4882:0:3:1.00:#NUM!: 0.00:#NUM!), (Salpingogram:0:4882:0:3:1.00:#NUM!:0.00:#NUM!), (Schwannoma:0:4882:0:3: 1.00:#NUM!:0.00:#NUM!), (Scopulariopsis.infection:0:4882:0:3:1.00:#NUM!:0.00:#NUM!), (Sc rotal.cyst:0:4882:0:3:1.00:#NUM!:0.00:#NUM!), (Scrub.typhus:0:4882:0:3:1.00:#NUM!:0.00:#NUM!), (Secondary.syphilis:0:4882:0:3:1.00:#NUM!:0.00:#NUM!), (Secondary.tic:0:4882:0:3:1. 00:#NUM!:0.00:#NUM!), (Separation.anxiety.disorder:0:4882:0:3:1.00:#NUM!:0.00:#NUM!), (S eptic.encephalopathy:0:4882:0:3:1.00:#NUM!:0.00:#NUM!), (Sexual.inhibition:0:4882:0:3:1.00: #NUM!:0.00:#NUM!), (Sexual.relationship.change:0:4882:0:3:1.00:#NUM!:0.00:#NUM!), (Skin. texture.abnormal:0:4882:0:3:1.00:#NUM!:0.00:#NUM!), (Sleep.sex:0:4882:0:3:1.00:#NUM!:0.00: #NUM!), (Small.intestine.operation:0:4882:0:3:1.00:#NUM!:0.00:#NUM!), (Sneddon's.syndro me:0:4882:0:3:1.00:#NUM!:0.00:#NUM!), (Spastic.diplegia:0:4882:0:3:1.00:#NUM!:0.00:#NU M!), (Spastic.paraplegia:0:4882:0:3:1.00:#NUM!:0.00:#NUM!), (Spermatozoa.progressive.motilit y.abnormal:0:4882:0:3:1.00:#NUM!:0.00:#NUM!), (Spinal. cord.oedema:0:4882:0:3:1.00:#NUM !:0.00:#NUM!), (Spur.cell.anaemia:0:4882:0:3:1.00:#NUM!:0.00:#NUM!), (Sucrose.intolerance: 0:4882:0:3:1.00:#NUM!:0.00:#NUM!), (Symbolic. dysfunction:0:4882:0:3:1.00:#NUM!:0.00:#N UM!), (Symphysiolysis:0:4882:0:3:1.00:#NUM!:0.00:#NUM!), (Synoviorthesis:0:4882:0:3:1.00: #NUM!:0.00:#NUM!), (Systolic.anterior.motion.of.mitral.valve:0:4882:0:3:1.00:#NUM!:0.00:#N UM!), (Tendon.laxity:0:4882:0:3:1.00:#NUM!:0.00:#NUM!), (Thyroxine:0:4882:0:3:1.00:#NUM !:0.00:#NUM!), (Tongue.carcinoma.stage.0:0:4882:0:3:1.00:#NUM!:0.00:#NUM!), (Tongue.carci noma.stage.I:0:4882:0:3:1.00:#NUM!:0.00:#NUM!), (Tongue.movement.disturbance:0:4882:0:3: 1.00:#NUM!:0.00:#NUM!), (Toxic.anterior.segment.syndrome:0:4882:0:3:1.00:#NUM!:0.00:#N UM!), (Tracheal.mass:0:4882:0:3:1.00:#NUM!:0.00:#NUM!), (Transaminases.abnormal:0:4882:0: 3:1.00:#NUM!:0.00:#NUM!), (Transplant.evaluation:0:4882:0:3:1.00:#NUM!:0.00:#NUM!), (Tr aumatic.haematoma:0:4882:0:3:1.00:#NUM!:0.00:#NUM!), (Trichodysplasia.spinulosa:0:4882:0: 3:1.00:#NUM!:0.00:#NUM!), (Trypanosomiasis:0:4882:0:3:1.00:#NUM!:0.00:#NUM!), (Tuberc ulous.tenosynovitis:0:4882:0:3:1.00:#NUM!:0.00:#NUM!), (Tumour.marker.abnormal:0:4882:0: 3:1.00:#NUM!:0.00:#NUM!), (Uterine.rupture:0:4882:0:3:1.00:#NUM!:0.00:#NUM!), (Vaginal.e rosion:0:4882:0:3:1.00:#NUM!:0.00:#NUM!), (Vagus.nerve.disorder:0:4882:0:3:1.00:#NUM!:0. 00:#NUM!), (Vascular.bypass. dysfunction:0:4882:0:3:1.00:#NUM!:0.00:#NUM!), (Vascular.sten osis:0:4882:0:3:1.00:#NUM!:0.00:#NUM!), (Vein.of.Galen.aneurysmal.malformation:0:4882:0:3: 1.00:#NUM!:0.00:#NUM!), (Very.low.density.lipoprotein.increased:0:4882:0:3:1.00:#NUM!:0. 00:#NUM!), (Violence-related.symptom:0:4882:0:3:1.00:#NUM!:0.00:#NUM!), (Viral.load.decreased:0:4882:0:3:1.00:#NUM!:0.00:#NUM!), (Visual.field.tests:0:4882:0:3:1.00:#NUM!:0.00:#NUM!), (Vocal.cord.poly p:0:4882:0:3:1.00:#NUM!:0.00:#NUM!), (Vomiting.psychogenic:0:4882:0:3:1.00:#NUM!:0.00:#NUM!), (Von.Hippel-Lindau.disease:0:4882:0:3:1.00:#NUM!:0.00:#NUM!), (Vulval.cancer.stage.0:0:4882:0:3:1.00:#NUM!:0.00:#NUM!), (Waldenstrom's.macroglobulinaemia.stage.II:0:4882:0:3:1.00:#NUM!:0.00: #NUM!), (Weight.loss.poor:0:4882:0:3:1.00:#NUM!:0.00:#NUM!), (Wound.sepsis:0:4882:0:3:1. 00:#NUM!:0.00:#NUM!), (X-ray.abnormal:0:4882:0:3:1.00:#NUM!:0.00:#NUM!), (X-ray.gastrointestinal.tract:0:4882:0:3:1.00:#NUM!:0.00:#NUM!), (Yawning:0:4882:0:3:1.00:#NU M!:0.00:#NUM!), (Yellow.fever.immunisation:0:4882:0:3:1.00:#NUM!:0.00:#NUM!), (Immunoa dsorption.therapy:11:4871:0:3:1.00:1.00:0.79:0.88), (ABO.incompatibility:10:4872:0:3:1.00:1.00: 0.77:0.87), (Hepatitis.B.antibody:5:4877:0:3:1.00:1.00:0.63:0.77), (Microsomia:5:4877:0:3:1.00: 1.00:0.63:0.77), (Nodal.marginal.zone.B-cell.lymphoma.stage.IV:5:4877:0:3:1.00:1.00:0.63:0.77), (Primitive.neuroectodermal.tumour.met astatic:5:4877:0:3:1.00:1.00:0.63:0.77), (Testicular.seminoma. (pure).stage.I:5:4877:0:3:1.00:1.00: 0.63:0.77), (Central.nervous.system.necrosis:4:4878:0:3:1.00:1.00:0.57:0.73), (Ovarian.germ.cell.endodermal.sinus.tumour.stage.IV:4:4878:0:3:1.00:1.00:0.57:0.73), (Pulmonary.tuberculoma:4:4 878:0:3:1.00:1.00:0.57:0.73), (Vasculitis.gastrointestinal:4:4878:0:3:1.00:1.00:0.57:0.73), (Vertic al.infection.transmission:4:4878:0:3:1.00:1.00:0.57:0.73), (Anti.B.antibody:3:4879:0:3:1.00:1.00: 0.50:0.67), (Autoantibody.positive:3:4879:0:3:1.00:1.00:0.50:0.67), (Benign.rolandic.epilepsy:3:4 879:0:3:1.00:1.00:0.50:0.67), (Delayed.delivery:3:4879:0:3:1.00:1.00:0.50:0.67), (Hippocampal.s clerosis:3:4879:0:3:1.00:1.00:0.50:0.67), (Interleukin.level.increased:3:4879:0:3:1.00:1.00:0.50:0 0.67), (Kaposi's.sarcoma.classical.type:3:4879:0:3:1.00:1.00:0.50:0.67), (Patellofemoral.pain.syndr ome:3:4879:0:3:1.00:1.00:0.50:0.67), (Sertoli.cell.testicular.tumour:3:4879:0:3:1.00:1.00:0.50:0. 67), (Small.cell.carcinoma.of.the.cervix:3:4879:0:3:1.00:1.00:0.50:0.67), (Waldenstrom's.macrogl obulinaemia.recurrent:3:4879:0:3:1.00:1.00:0.50:0.67), (Acral.lentiginous.melanoma.stage.IV:2:4 880:0:3:1.00:1.00:0.40:0.57), (Angiocentric.glioma:2:4880:0:3:1.00:1.00:0.40:0.57), (Atypical.be nign.partial.epilepsy:2:4880:0:3:1.00:1.00:0.40:0.57), (Bladder.perforation:2:4880:0:3:1.00:1.00: 0.40:0.57), (Blood.urea:2:4880:0:3:1.00:1.00:0.40:0.57), (Cervical.cyst:2:4880:0:3:1.00:1.00:0.40: 0.57), (Extranodal.marginal. zone.B-cell.lymphoma.(MALT.type).stage.IV:2:4880:0:3:1.00:1.00:0.40:0.57), (Feeding.disorder.of.infa ncy.or.early.childhood:2:4880:0:3:1.00:1.00:0.40:0.57), (Fibula.fracture:2:4880:0:3:1.00:1.00:0.4 0:0.57), (Flagellate.dermatitis:2:4880:0:3:1.00:1.00:0.40:0.57), (Glucose.urine:2:4880:0:3:1.00:1. 00:0.40:0.57), (Human.polyomavirus.infection:2:4880:0:3:1.00:1.00:0.40:0.57), (Hyperthermia.th erapy:2:4880:0:3:1.00:1.00:0.40:0.57), (Iris.neovascularisation:2:4880:0:3:1.00:1.00:0.40:0.57), (Measles:2:4880:0:3:1.00:1.00:0.40:0.57), (Mucoepidermoid.carcinoma:2:4880:0:3:1.00:1.00:0.40:0.57), (Muscular.sarcoidosis:2:4880:0:3:1.00:1.00:0.40:0.57), (Myosclerosis:2:4880:0:3:1.00:1.0 0:0.40:0.57), (Ortner's.syndrome:2:4880:0:3:1.00:1.00:0.40:0.57), (Pneumonic.plague:2:4880:0:3: 1.00:1.00:0.40:0.57), (Pulmonary.endarterectomy:2:4880:0:3:1.00:1.00:0.40:0.57), (Secondary.cer ebellar.degeneration:2:4880:0:3:1.00:1.00:0.40:0.57), (Sweat.gland.tumour:2:4880:0:3:1.00:1.00: 0.40:0.57), (Testicular.seminoma.(pure).stage.II:2:4880:0:3:1.00:1.00:0.40:0.57), (Tracheo-oesophageal.fistula:2:4880:0:3:1.00:1.00:0.40:0.57), (Virologic.failure:2:4880:0:3:1.00:1.00:0.40: 0.57), (Wolf-Hirschhom.syndrome:2:4880:0:3:1.00:1.00:0.40:0.57), (Abortion.complete:1:4881:0:3:1.00:1.00: 0.25:0.40), (Adenoviral.hepatitis:1:4881:0:3:1.00:1.00:0.25:0.40), (Adjustment.disorder.with.mixe d.disturbance.of.emotion.and.conduct:1:4881:0:3:1.00:1.00:0.25:0.40), (Administration.site.reacti on:1:4881:0:3:1.00:1.00:0.25:0.40), (Anti-interferon.antibody.positive:1:4881:0:3:1.00:1.00:0.25:0.40), (Anti.factor.V.antibody.positive:1:4 881:0:3:1.00:1.00:0.25:0.40), (Apnoeic.attack:1:4881:0:3:1.00:1.00:0.25:0.40), (Arteriosclerotic.r etinopathy:1:4881:0:3:1.00:1.00:0.25:0.40), (Arthritis.allergic:1:4881:0:3:1.00:1.00:0.25:0.40), (A utomatism.epileptic:1:4881:0:3:1.00:1.00:0.25:0.40), (Basilar.artery.thrombosis:1:4881:0:3:1.00: 1.00:0.25:0.40), (Benign.lymph.node.neoplasm:1:4881:0:3:1.00:1.00:0.25:0.40), (Benign.vascular.neoplasm:1:4881:0:3:1.00:1.00:0.25:0.40), (Blood.heavy.metal.abnormal:1:4881:0:3:1.00:1.00:0 0.25:0.40), (Bone.marrow.necrosis:1:4881:0:3:1.00:1.00:0.25:0.40), (Breast.discharge:1:4881:0:3:1 0.00:1.00:0.25:0.40), (Breast.sarcoma:1:4881:0:3:1.00:1.00:0.25:0.40), (Brenner.tumour:1:4881:0: 3:1.00:1.00:0.25:0.40), (Carotid.arterial.embolus:1:4881:0:3:1.00:1.00:0.25:0.40), (Chemical.bum.of.skin:1:4881:0:3:1.00:1.00:0.25:0.40), (Choroid.plexus.papilloma:1:4881:0:3:1.00:1.00:0.25:0. 40), (Comeal.degeneration:1:4881:0:3:1.00:1.00:0.25:0.40), (Cytogenetic.analysis.abnormal:1:48 81:0:3:1.00:1.00:0.25:0.40), (Dermatitis.papillaris.capillitii:1:4881:0:3:1.00:1.00:0.25:0.40), (Dias tasis.recti.abdominis:1:4881:0:3:1.00:1.00:0.25:0.40), (Dysmorphophobia:1:4881:0:3:1.00:1.00:0 0.25:0.40), (Epiduroscopy:1:4881:0:3:1.00:1.00:0.25:0.40), (Failed.induction.of.labour:1:4881:0:3: 1.00:1.00:0.25:0.40), (False.positive.tuberculosis.test:1:4881:0:3:1.00:1.00:0.25:0.40), (Fibrous.dy splasia.of.jaw:1:4881:0:3:1.00:1.00:0.25:0.40), (Gamma-glutamyltransferase:1:4881:0:3:1.00:1.00:0.25:0.40), (Genital.cancer.male:1:4881:0:3:1.00:1.00:0 0.25:0.40), (Genital.labial.adhesions:1:4881:0:3:1.00:1.00:0.25:0.40), (Glutathione.synthetase.defic iency:1:4881:0:3:1.00:1.00:0.25:0.40), (Hangnail:1:4881:0:3:1.00:1.00:0.25:0.40), (Hepatitis.B.an tibody.positive:1:4881:0:3:1.00:1.00:0.25:0.40), (Histiocytic.necrotising.lymphadenitis:1:4881:0: 3:1.00:1.00:0.25:0.40), (Hypochondroplasia:1:4881:0:3:1.00:1.00:0.25:0.40), (Inguinal.mass:1:48 81:0:3:1.00:1.00:0.25:0.40), (Injection.site.haemorrhage:1:4881:0:3:1.00:1.00:0.25:0.40), (Interle ukin.therapy:1:4881:0:3:1.00:1.00:0.25:0.40), (Iris.incarceration:1:4881:0:3:1.00:1.00:0.25:0.40), (Leukoderma:1:4881:0:3:1.00:1.00:0.25:0.40), (Limb.fracture:1:4881:0:3:1.00:1.00:0.25:0.40), (L iposuction:1:4881:0:3:1.00:1.00:0.25:0.40), (Loeffler's.syndrome:1:4881:0:3:1.00:1.00:0.25:0.40), (Magnetic.resonance.neurography:1:4881:0:3:1.00:1.00:0.25:0.40), (Malignant.breast.lump.remo val:1:4881:0:3:1.00:1.00:0.25:0.40), (Medication.overuse.headache:1:4881:0:3:1.00:1.00:0.25:0.4 0), (Megakaryocytes.decreased:1:4881:0:3:1.00:1.00:0.25:0.40), (Mesenteric.neoplasm:1:4881:0: 3:1.00:1.00:0.25:0.40), (Mesothelioma.malignant.recurrent:1:4881:0:3:1.00:1.00:0.25:0.40), (Mitr al.valve.disease.mixed:1:4881:0:3:1.00:1.00:0.25:0.40), (Necrotising.herpetic.retinopathy:1:4881: 0:3:1.00:1.00:0.25:0.40), (Non-obstructive.cardiomyopathy:1:4881:0:3:1.00:1.00:0.25:0.40), (Obstructed.labour:1:4881:0:3:1.00: 1.00:0.25:0.40), (Oral.cavity.cancer.metastatic:1:4881:0:3:1.00:1.00:0.25:0.40), (Osteosis:1:4881: 0:3:1.00:1.00:0.25:0.40), (Pancoast's.tumour:1:4881:0:3:1.00:1.00:0.25:0.40), (Paroxysmal.choreo athetosis:1:4881:0:3:1.00:1.00:0.25:0.40), (Peri-implantitis:1:4881:0:3:1.00:1.00:0.25:0.40), (Pericarditis.constrictive:1:4881:0:3:1.00:1.00:0.25:0 0.40), (Perineal.disorder:1:4881:0:3:1.00:1.00:0.25:0.40), (Peripheral.artery.dissection:1:4881:0:3:1 0.00:1.00:0.25:0.40), (Periphlebitis:1:4881:0:3:1.00:1.00:0.25:0.40), (Pleuropericarditis:1:4881:0:3: 1.00:1.00:0.25:0.40), (Post.infection.glomerulonephritis:1:4881:0:3:1.00:1.00:0.25:0.40), (Postpar tum.venous.thrombosis:1:4881:0:3:1.00:1.00:0.25:0.40), (Premature.ovulation:1:4881:0:3:1.00:1. 00:0.25:0.40), (Progressive.bulbar.palsy:1:4881:0:3:1.00:1.00:0.25:0.40), (Prostate.cancer.stage.I: 1:4881:0:3:1.00:1.00:0.25:0.40), (Pulmonary.arterial.wedge.pressure.increased:1:4881:0:3:1.00:1. 00:0.25:0.40), (Removal.of.foreign.body.from.external.ear:1:4881:0:3:1.00:1.00:0.25:0.40), (Rena 1.vein.embolism:1:4881:0:3:1.00:1.00:0.25:0.40), (Retinal.cyst:1:4881:0:3:1.00:1.00:0.25:0.40), (Risk.of.future.pregnancy.miscarriage:1:4881:0:3:1.00:1.00:0.25:0.40), (Scleromalacia:1:4881:0:3: 1.00:1.00:0.25:0.40), (Sick.relative:1:4881:0:3:1.00:1.00:0.25:0.40), (Sideropenic.dysphagia:1:48 81:0:3:1.00:1.00:0.25:0.40), (Spinal.shock:1:4881:0:3:1.00:1.00:0.25:0.40), (Sputum.decreased:1: 4881:0:3:1.00:1.00:0.25:0.40), (Stewart-Treves.syndrome:1:4881:0:3:1.00:1.00:0.25:0.40), (Sudden.death:1:4881:0:3:1.00:1.00:0.25:0.40), (Terminal.state:1:4881:0:3:1.00:1.00:0.25:0.40), (Testotoxicosis:1:4881:0:3:1.00:1.00:0.25:0.40), (Tracheal.disorder:1:4881:0:3:1.00:1.00:0.25:0.40), (Tracheo-oesophageal.puncture:1:4881:0:3:1.00:1.00:0.25:0.40), (Transitional.cell.cancer.of.the.renal.pelvi s.and.ureter.localised:1:4881:0:3:1.00:1.00:0.25:0.40), (Urethral.stent.insertion:1:4881:0:3:1.00:1 0.00:0.25:0.40), (Urine.calcium.decreased:1:4881:0:3:1.00:1.00:0.25:0.40), (Uterine.haematoma:1: 4881:0:3:1.00:1.00:0.25:0.40), (Victim.of.child.abuse:1:4881:0:3:1.00:1.00:0.25:0.40), (Vitamin. B6:1:4881:0:3:1.00:1.00:0.25:0.40), (Vocal.cord.neoplasm:1:4881:0:3:1.00:1.00:0.25:0.40), (Wou nd.infection.fungal:1:4881:0:3:1.00:1.00:0.25:0.40), (Abscess.soft.tissue:0:4881:0:4:1.00:#NUM!: 0.00:#NUM!), (Acne.infantile:0:4881:0:4:1.00:#NUM!:0.00:#NUM!), (Action.tremor:0:4881:0:4 1.00:#NUM!:0.00:#NUM!), (Acute.post.asthmatic.amyotrophy:0:4881:0:4:1.00:#NUM!:0.00:#N UM!), (Aeromona.infection:0:4881:0:4:1.00:#NUM!:0.00:#NUM!), (Amniorrhoea:0:4881:0:4:1.0 0:#NUM!:0.00:#NUM!), (Anal.dilatation:0:4881:0:4:1.00:#NUM!:0.00:#NUM!), (Anastomotic.le ak:0:4881:0:4:1.00:#NUM!:0.00:#NUM!), (Anticoagulation.drug.level.increased:0:4881:0:4:1.00: #NUM!:0.00:#NUM!), (Antipsychotic.drug.level.above.therapeutic:0:4881:0:4:1.00:#NUM!:0.0 0:#NUM!), (Apical.granuloma:0:4881:0:4:1.00:#NUM!:0.00:#NUM!), (Appendix.disorder:0:488 1:0:4:1.00:#NUM!:0.00:#NUM!), (Application.site.infection:0:4881:0:4:1.00:#NUM!:0.00:#NU M!), (Application.site.plaque:0:4881:0:4:1.00:#NUM!:0.00:#NUM!), (Arterial.switch.operation:0: 4881:0:4:1.00:#NUM!:0.00:#NUM!), (Articular.calcification:0:4881:0:4:1.00:#NUM!:0.00:#NU M!), (Atrophic.thyroiditis:0:4881:0:4:1.00:#NUM!:0.00:#NUM!), (Bacteroides.bacteraemia:0:488 1:0:4:1.00:#NUM!:0.00:#NUM!), (Bankruptcy:0:4881:0:4:1.00:#NUM!:0.00:#NUM!), (Bartholin's.abscess:0:4881:0:4:1.00:#NUM!:0.00:#NUM!), (Benign.familial.neonatal.convulsions:0:4881: 0:4:1.00:#NUM!:0.00:#NUM!), (Biopsy.bladder:0:4881:0:4:1.00:#NUM!:0.00:#NUM!), (Bladder.adenocarcinoma.stage.IV:0:4881:0:4:1.00:#NUM!:0.00:#NUM!), (Bladder.sphincterectomy:0:48 81:0:4:1.00:#NUM!:0.00:#NUM!), (Blebitis:0:4881:0:4:1.00:#NUM!:0.00:#NUM!), (Blepharal.p apilloma:0:4881:0:4:1.00:#NUM!:0.00:#NUM!), (Blepharitis.allergic:0:4881:0:4:1.00:#NUM!:0. 00:#NUM!), (Blood.aldosterone.decreased:0:4881:0:4:1.00:#NUM!:0.00:#NUM!), (Blood.alkalin e.phosphatase.normal:0:4881:0:4:1.00:#NUM!:0.00:#NUM!), (Blood.calcitonin.increased:0:4881: 0:4:1.00:#NUM!:0.00:#NUM!), (Blood.cholinesterase.increased:0:4881:0:4:1.00:#NUM!:0.00:#NUM!), (Blood.ethanol.increased:0:4881:0:4:1.00:#NUM!:0.00:#NUM!), (Blood.glucagon.increa sed:0:4881:0:4:1.00:#NUM!:0.00:#NUM!), (Blood.pressure.diastolic.decreased:0:4881:0:4:1.00: #NUM!:0.00:#NUM!), (Borderline.leprosy:0:4881:0:4:1.00:#NUM!:0.00:#NUM!), (Boutonneuse. fever:0:4881:0:4:1.00:#NUM!:0.00:#NUM!), (Bowenoid.papulosis:0:4881:0:4:1.00:#NUM!:0.00: #NUM!), (Brain.stem.ischaemia:0:4881:0:4:1.00:#NUM!:0.00:#NUM!), (Brevibacterium.infectio n:0:4881:0:4:1.00:#NUM!:0.00:#NUM!), (Calcification.metastatic:0:4881:0:4:1.00:#NUM!:0.00: #NUM!), (Calculus.prostatic:0:4881:0:4:1.00:#NUM!:0.00:#NUM!), (Cardiac.function.test:0:488 1:0:4:1.00:#NUM!:0.00:#NUM!), (Cardiac.monitoring:0:4881:0:4:1.00:#NUM!:0.00:#NUM!), (C arotid.angioplasty:0:4881:0:4:1.00:#NUM!:0.00:#NUM!), (Cartilage.neoplasm:0:4881:0:4:1.00:#NUM!:0.00:#NUM!), (Central.nervous.system.melanoma:0:4881:0:4:1.00:#NUM!:0.00:#NUM!), (Cerebellar.cognitive.affective.syndrome:0:4881:0:4:1.00:#NUM!:0.00:#NUM!), (Cerebrovascul ar.operation:0:4881:0:4:1.00:#NUM!:0.00:#NUM!), (Chronic.tonsillitis:0:4881:0:4:1.00:#NUM!: 0.00:#NUM!), (Citric.acid.urine.decreased:0:4881:0:4:1.00:#NUM!:0.00:#NUM!), (Computer. visi on.syndrome:0:4881:0:4:1.00:#NUM!:0.00:#NUM!), (Computerised.tomogram.kidney:0:4881:0: 4:1.00:#NUM!:0.00:#NUM!), (Congenital.aortic.valve.incompetence:0:4881:0:4:1.00:#NUM!:0. 00:#NUM!), (Congenital.melanocytic.naevus:0:4881:0:4:1.00:#NUM!:0.00:#NUM!), (Congenital.nystagmus:0:4881:0:4:1.00:#NUM!:0.00:#NUM!), (Copper.metabolism.disorder:0:4881:0:4:1.0 0:#NUM!:0.00:#NUM!), (Corneal.epithelium.defect:0:4881:0:4:1.00:#NUM!:0.00:#NUM!), (Cor neal.implant:0:4881:0:4:1.00:#NUM!:0.00:#NUM!), (Corneal.thickening:0:4881:0:4:1.00:#NUM !:0.00:#NUM!), (CSF.virus.identified:0:4881:0:4:1.00:#NUM!:0.00:#NUM!), (Cystitis.pseudomo nal:0:4881:0:4:1.00:#NUM!:0.00:#NUM!), (Defaecation.disorder:0:4881:0:4:1.00:#NUM!:0.00:#NUM!), (Dependent.rubor:0:4881:0:4:1.00:#NUM!:0.00:#NUM!), (Device.dependence:0:4881:0: 4:1.00:#NUM!:0.00:#NUM!), (Diabetic.amyotrophy:0:4881:0:4:1.00:#NUM!:0.00:#NUM!), (Dru g.ineffective.for.unapproved.indication:0:4881:0:4:1.00:#NUM!:0.00:#NUM!), (Ear.canal.stenosi s:0:4881:0:4:1.00:#NUM!:0.00:#NUM!), (Ejaculation.disorder:0:4881:0:4:1.00:#NUM!:0.00:#N UM!), (Electrocardiogram.ST.segment.abnormal:0:4881:0:4:1.00:#NUM!:0.00:#NUM!), (Emotio nal.disorder.of.childhood:0:4881:0:4:1.00:#NUM!:0.00:#NUM!), (Encephalopathy.neonatal:0:48 81:0:4:1.00:#NUM!:0.00:#NUM!), (Eosinopenia:0:4881:0:4:1.00:#NUM!:0.00:#NUM!), (Epider modysplasia.verruciformis:0:4881:0:4:1.00:#NUM!:0.00:#NUM!), (Exocrine.pancreatic.function. test.abnormal:0:4881:0:4:1.00:#NUM!:0.00:#NUM!), (Expanded.disability.status.scale:0:4881:0: 4:1.00:#NUM!:0.00:#NUM!), (Eyelid.dermatochalasis:0:4881:0:4:1.00:#NUM!:0.00:#NUM!), (F actor.VII.inhibition:0:4881:0:4:1.00:#NUM!:0.00:#NUM!), (Fallopian.tube.cyst:0:4881:0:4:1.00: #NUM!:0.00:#NUM!), (Familial.hypocalciuric.hypercalcaemia:0:4881:0:4:1.00:#NUM!:0.00:#N UM!), (Fascioliasis:0:4881:0:4:1.00:#NUM!:0.00:#NUM!), (Fingerprint.loss:0:4881:0:4:1.00:#N UM!:0.00:#NUM!), (Follicle.centre.lymphoma,.follicular.grade.I,.II,.III.stage.I:0:4881:0:4:1.00:#NUM!:0.00:#NUM!), (Forced.vital.capacity.decreased:0:4881:0:4:1.00:#NUM!:0.00:#NUM!), (F orceps.delivery:0:4881:0:4:1.00:#NUM!:0.00:#NUM!), (Frenulum.breve:0:4881:0:4:1.00:#NUM !:0.00:#NUM!), (Genotype.drug.resistance.test.positive:0:4881:0:4:1.00:#NUM!:0.00:#NUM!), (Gingival.blister:0:4881:0:4:1.00:#NUM!:0.00:#NUM!), (Gingival.operation:0:4881:0:4:1.00:#N UM!:0.00:#NUM!), (Globulins.increased:0:4881:0:4:1.00:#NUM!:0.00:#NUM!), (Gram.stain.pos itive:0:4881:0:4:1.00:#NUM!:0.00:#NUM!), (Granulomatous.rosacea:0:4881:0:4:1.00:#NUM!:0. 00:#NUM!), (Grey.Turner's.sign:0:4881:0:4:1.00:#NUM!:0.00:#NUM!), (Haemorrhagic.cyst:0:48 81:0:4:1.00:#NUM!:0.00:#NUM!), (Heat.therapy:0:4881:0:4:1.00:#NUM!:0.00:#NUM!), (Herpes.zoster.necrotising.retinopathy:0:4881:0:4:1.00:#NUM!:0.00:#NUM!), (HIV.antigen:0:4881:0:4:1 0.00:#NUM!:0.00:#NUM!), (HIV.tropism.test:0:4881:0:4:1.00:#NUM!:0.00:#NUM!), (Hodgkin's. disease.lymphocyte.predominance.stage.I.site.unspec:0:4881:0:4:1.00:#NUM!:0.00:#NUM!), (H odgkin's.disease.lymphocyte.predominance.stage.I.supradiaphragm:0:4881:0:4:1.00:#NUM!:0.0 0:#NUM!), (Human.T-cell.lymphotropic.virus.infection:0:4881:0:4:1.00:#NUM!:0.00:#NUM!), (Hyaluronic.acid.abnor mal:0:4881:0:4:1.00:#NUM!:0.00:#NUM!), (Hydrothorax:0:4881:0:4:1.00:#NUM!:0.00:#NUM!), (Hyperbaric.oxygen.therapy:0:4881:0:4:1.00:#NUM!:0.00:#NUM!), (Hyperkinetic.heart.syndro me:0:4881:0:4:1.00:#NUM!:0.00:#NUM!), (Hyperosmolar.hyperglycaemic.state:0:4881:0:4:1.00: #NUM!:0.00:#NUM!), (Hypnopompic.hallucination:0:4881:0:4:1.00:#NUM!:0.00:#NUM!), (Hy sterosalpingogram:0:4881:0:4:1.00:#NUM!:0.00:#NUM!), (Hysterotomy:0:4881:0:4:1.00:#NUM !:0.00:#NUM!), (Inbom.error.of.bilirubin.metabolism:0:4881:0:4:1.00:#NUM!:0.00:#NUM!), (Inf ected.neoplasm:0:4881:0:4:1.00:#NUM!:0.00:#NUM!), (Injection.site.abscess:0:4881:0:4:1.00:#NUM!:0.00:#NUM!), (Intestinal.dilatation:0:4881:0:4:1.00:#NUM!:0.00:#NUM!), (Intrapartum.h aemorrhage:0:4881:0:4:1.00:#NUM!:0.00:#NUM!), (Iodine.uptake.abnormal:0:4881:0:4:1.00:#N UM!:0.00:#NUM!), (Jarisch-Herxheimer.reaction:0:4881:0:4:1.00:#NUM!:0.00:#NUM!), (Kinesitherapy:0:4881:0:4:1.00:#N UM!:0.00:#NUM!), (Laryngeal.cancer.stage.0:0:4881:0:4:1.00:#NUM!:0.00:#NUM!), (Left.atrial.enlargement:0:4881:0:4:1.00:#NUM!:0.00:#NUM!), (Left.ventricular.enlargement:0:4881:0:4:1. 00:#NUM!:0.00:#NUM!), (Leukaemia.granulocytic:0:4881:0:4:1.00:#NUM!:0.00:#NUM!), (Liga ment.laxity:0:4881:0:4:1.00:#NUM!:0.00:#NUM!), (Limb.prosthesis.user:0:4881:0:4:1.00:#NU M!:0.00:#NUM!), (Lumbosacral.plexopathy:0:4881:0:4:1.00:#NUM!:0.00:#NUM!), (Lymphocyt e.count.abnormal:0:4881:0:4:1.00:#NUM!:0.00:#NUM!), (Malignant.mesenchymoma:0:4881:0:4: 1.00:#NUM!:0.00:#NUM!), (Malignant.neoplasm.of.lacrimal.duct:0:4881:0:4:1.00:#NUM!:0.00: #NUM!), (Marchiafava-Bignami.disease:0:4881:0:4:1.00:#NUM!:0.00:#NUM!), (Matrixectomy:0:4881:0:4:1.00:#NUM!: 0.00:#NUM!), (Mediastinal.haemorrhage:0:4881:0:4:1.00:#NUM!:0.00:#NUM!), (Mendelson's.s yndrome:0:4881:0:4:1.00:#NUM!:0.00:#NUM!), (Meningitis.salmonella:0:4881:0:4:1.00:#NUM: 0.00:#NUM!), (Metastases.to.nasal.sinuses:0:4881:0:4:1.00:#NUM!:0.00:#NUM!), (Mineraloco rticoid.deficiency:0:4881:0:4:1.00:#NUM!:0.00:#NUM!), (Mucosal.discolouration:0:4881:0:4:1. 00:#NUM!:0.00:#NUM!), (Myelomalacia:0:4881:0:4:1.00:#NUM!:0.00:#NUM!), (Myxoedema.c oma:0:4881:0:4:1.00:#NUM!:0.00:#NUM!), (Narcissistic.personality.disorder:0:4881:0:4:1.00:#NUM!:0.00:#NUM!), (Nasal.injury:0:4881:0:4:1.00:#NUM!:0.00:#NUM!), (Nasopharyngeal.can cer.stage.II:0:4881:0:4:1.00:#NUM!:0.00:#NUM!), (Neck.crushing:0:4881:0:4:1.00:#NUM!:0.00: #NUM!), (Neonatal.aspiration:0:4881:0:4:1.00:#NUM!:0.00:#NUM!), (Neonatal.cardiac.failure: 0:4881:0:4:1.00:#NUM!:0.00:#NUM!), (Neoplasm.of.appendix:0:4881:0:4:1.00:#NUM!:0.00:#N UM!), (Nephritis.allergic:0:4881:0:4:1.00:#NUM!:0.00:#NUM!), (Nose.deformity:0:4881:0:4:1.0 0:#NUM!:0.00:#NUM!), (Occupational.exposure.to.dust:0:4881:0:4:1.00:#NUM!:0.00:#NUM!), (Occupational.exposure.to.toxic.agent:0:4881:0:4:1.00:#NUM!:0.00:#NUM!), (Olfactory.nerve.di sorder:0:4881:0:4:1.00:#NUM!:0.00:#NUM!), (Oral.administration.complication:0:4881:0:4:1.00: #NUM!:0.00:#NUM!), (Orbital.oedema:0:4881:0:4:1.00:#NUM!:0.00:#NUM!), (Orgasm.abnorm al:0:4881:0:4:1.00:#NUM!:0.00:#NUM!), (Osteitis.condensans:0:4881:0:4:1.00:#NUM!:0.00:#N UM!), (Ovarian.atrophy:0:4881:0:4:1.00:#NUM!:0.00:#NUM!), (Pacemaker.generated.rhythm:0: 4881:0:4:1.00:#NUM!:0.00:#NUM!), (Pancreatic.atrophy:0:4881:0:4:1.00:#NUM!:0.00:#NUM!), (Parametritis:0:4881:0:4:1.00:#NUM!:0.00:#NUM!), (Parotid.abscess:0:4881:0:4:1.00:#NUM!:0 0.00:#NUM!), (Peak.expiratory.flow.rate.decreased:0:4881:0:4:1.00:#NUM!:0.00:#NUM!), (Pelvic.deformity:0:4881:0:4:1.00:#NUM!:0.00:#NUM!), (Penile.oedema:0:4881:0:4:1.00:#NUM!:0.00: #NUM!), (Perineotomy:0:4881:0:4:1.00:#NUM!:0.00:#NUM!), (Pharyngeal.neoplasm.benign:0:4 881:0:4:1.00:#NUM!:0.00:#NUM!), (Pharyngeal.reconstruction:0:4881:0:4:1.00:#NUM!:0.00:#NUM!), (Placental.necrosis:0:4881:0:4:1.00:#NUM!:0.00:#NUM!), (Platelet-derived.growth.factor.receptor.gene.mutation:0:4881:0:4:1.00:#NUM!:0.00:#NUM!), (Porocarcin oma:0:4881:0:4:1.00:#NUM!:0.00:#NUM!), (Portosplenomesenteric.venous.thrombosis:0:4881:0: 4:1.00:#NUM!:0.00:#NUM!), (Post.procedural.fistula:0:4881:0:4:1.00:#NUM!:0.00:#NUM!), (P ost.treatment.Lyme.disease.syndrome:0:4881:0:4:1.00:#NUM!:0.00:#NUM!), (Postoperative.ileu s:0:4881:0:4:1.00:#NUM!:0.00:#NUM!), (Postpartum.stress.disorder:0:4881:0:4:1.00:#NUM!:0. 00:#NUM!), (Postresuscitation.encephalopathy:0:4881:0:4:1.00:#NUM!:0.00:#NUM!), (Premenst rual.cramps:0:4881:0:4:1.00:#NUM!:0.00:#NUM!), (Presbyoesophagus:0:4881:0:4:1.00:#NUM!: 0.00:#NUM!), (Primary.hypoparathyroidism:0:4881:0:4:1.00:#NUM!:0.00:#NUM!), (Primary.me diastinal.large.B-cell.lymphoma.refractory:0:4881:0:4:1.00:#NUM!:0.00:#NUM!), (Procedural.dizziness:0:4881:0: 4:1.00:#NUM!:0.00:#NUM!), (Product.packaging.issue:0:4881:0:4:1.00:#NUM!:0.00:#NUM!), (Product.selection.error:0:4881:0:4:1.00:#NUM!:0.00:#NUM!), (Prostatic.cyst:0:4881:0:4:1.00:#NUM!:0.00:#NUM!), (Protein.S.decreased:0:4881:0:4:1.00:#NUM!:0.00:#NUM!), (Psychogenic. visual.disorder:0:4881:0:4:1.00:#NUM!:0.00:#NUM!), (Pulmonary. bulla:0:4881:0:4:1.00:#NUM: 0.00:#NUM!), (Pulmonary.cavitation:0:4881:0:4:1.00:#NUM!:0.00:#NUM!), (Radical.cystecto my:0:4881:0:4:1.00:#NUM!:0.00:#NUM!), (Radiculitis.cervical:0:4881:0:4:1.00:#NUM!:0.00:#NUM!), (Radiolucency.around. implant:0:4881:0:4:1.00:#NUM!:0.00:#NUM!), (Reaction.to.prese rvatives:0:4881:0:4:1.00:#NUM!:0.00:#NUM!), (Rectal.examination:0:4881:0:4:1.00:#NUM!:0. 00:#NUM!), (Rectosigmoid.cancer.stage.I:0:4881:0:4:1.00:#NUM!:0.00:#NUM!), (Removal.of.fo reign.body.from.eye:0:4881:0:4:1.00:#NUM!:0.00:#NUM!), (Respiratory.gas. exchange. disorder: 0:4881:0:4:1.00:#NUM!:0.00:#NUM!), (Retroperitoneal.effusion:0:4881:0:4:1.00:#NUM!:0.00:#NUM!), (Rhinosporidiosis:0:4881:0:4:1.00:#NUM!:0.00:#NUM!), (Round.cell.liposarcoma:0:488 1:0:4:1.00:#NUM!:0.00:#NUM!), (Salmonella.test.positive:0:4881:0:4:1.00:#NUM!:0.00:#NUM !), (Scoliosis.surgery:0:4881:0:4:1.00:#NUM!:0.00:#NUM!), (Shock.hypoglycaemic:0:4881:0:4:1 0.00:#NUM!:0.00:#NUM!), (Sinusitis.noninfective:0:4881:0:4:1.00:#NUM!:0.00:#NUM!), (Skin.l axity:0:4881:0:4:1.00:#NUM!:0.00:#NUM!), (Skin.oedema:0:4881:0:4:1.00:#NUM!:0.00:#NUM !), (Social.fear:0:4881:0:4:1.00:#NUM!:0.00:#NUM!), (Spinal.manipulation:0:4881:0:4:1.00:#NU M!:0.00:#NUM!), (Surgical.fixation.of.rib.fracture:0:4881:0:4:1.00:#NUM!:0.00:#NUM!), (Syno vectomy:0:4881:0:4:1.00:#NUM!:0.00:#NUM!), (Tachycardia.induced.cardiomyopathy:0:4881:0: 4:1.00:#NUM!:0.00:#NUM!), (Term.birth:0:4881:0:4:1.00:#NUM!:0.00:#NUM!), (Testicular.cy st:0:4881:0:4:1.00:#NUM!:0.00:#NUM!), (Throat.lesion:0:4881:0:4:1.00:#NUM!:0.00:#NUM!), (Tinea.barbae:0:4881:0:4:1.00:#NUM!:0.00:#NUM!), (Tongue.discomfort:0:4881:0:4:1.00:#NU M!:0.00:#NUM!), (Toxocariasis:0:4881:0:4:1.00:#NUM!:0.00:#NUM!), (Trichiniasis:0:4881:0:4: 1.00:#NUM!:0.00:#NUM!), (Ultrasound.prostate:0:4881:0:4:1.00:#NUM!:0.00:#NUM!), (Ultraso und. scan. abnormal:0:4881:0:4:1.00:#NUM!:0.00:#NUM!), (Underdose:0:4881:0:4:1.00:#NUM!: 0.00:#NUM!), (Upper.respiratory.tract.endoscopy:0:4881:0:4:1.00:#NUM!:0.00:#NUM!), (Ureapl asma.test.positive:0:4881:0:4:1.00:#NUM!:0.00:#NUM!), (Ureteral.catheterisation:0:4881:0:4:1. 00:#NUM!:0.00:#NUM!), (Urogenital.infection.fungal:0:4881:0:4:1.00:#NUM!:0.00:#NUM!), (V aginal.cancer.stage.0:0:4881:0:4:1.00:#NUM!:0.00:#NUM!), (Varicose.vein.ruptured:0:4881:0:4: 1.00:#NUM!:0.00:#NUM!), (Venous.ligation:0:4881:0:4:1.00:#NUM!:0.00:#NUM!), (Vertebral.a rtery.occlusion:0:4881:0:4:1.00:#NUM!:0.00:#NUM!), (Vulva.cyst:0:4881:0:4:1.00:#NUM!:0.00: #NUM!), (Vulvectomy:0:4881:0:4:1.00:#NUM!:0.00:#NUM!), (Vulvovaginal.rash:0:4881:0:4:1. 00:#NUM!:0.00:#NUM!), (White.blood.cells.semen.positive:0:4881:0:4:1.00:#NUM!:0.00:#NU M!), (Williams.syndrome:0:4881:0:4:1.00:#NUM!:0.00:#NUM!), (Xanthoma:0:4881:0:4:1.00:#N UM!:0.00:#NUM!), (Yersinia.infection:0:4881:0:4:1.00:#NUM!:0.00:#NUM!), (Zika.virus.infecti on:0:4881:0:4:1.00:#NUM!:0.00:#NUM!), (Hodgkin's.disease. mixed. cellularity.stage.III:11:4870: 0:4:1.00:1.00:0.73:0.85), (Primary.cardiac.lymphoma:8:4873:0:4:1.00:1.00:0.67:0.80), (Genitouri nary.tract.gonococcal.infection:7:4874:0:4:1.00:1.00:0.64:0.78), (Idiopathic.orbital.inflammation: 7:4874:0:4:1.00:1.00:0.64:0.78), (Testicular.choriocarcinoma:7:4874:0:4:1.00:1.00:0.64:0.78), (R ectal.stenosis:6:4875:0:4:1.00:1.00:0.60:0.75), (Tuberculosis.of.eye:6:4875:0:4:1.00:1.00:0.60:0. 75), (Extragonadal.primary.seminoma. (pure):5:4876:0:4:1.00:1.00:0.56:0.71), (Ovarian.germ.cell. endodermal.sinus.tumour:5:4876:0:4:1.00:1.00:0.56:0.71), (Pharyngeal.neoplasm:5:4876:0:4:1.0 0:1.00:0.56:0.71), (Enteropathic.spondylitis:4:4877:0:4:1.00:1.00:0.50:0.67), (Epstein-Barr.virus.test:4:4877:0:4:1.00:1.00:0.50:0.67), (Maternal.use.of.illicit.drugs:4:4877:0:4:1.00:1.0 0:0.50:0.67), (Renal.tuberculosis:4:4877:0:4:1.00:1.00:0.50:0.67), (Spinal.fusion.acquired:4:4877: 0:4:1.00:1.00:0.50:0.67), (Splenic.marginal.zone.lymphoma.stage.IV:4:4877:0:4:1.00:1.00:0.50:0 0.67), (Abortion.missed:3:4878:0:4:1.00:1.00:0.43:0.60), (Acquired.antithrombin.III.deficiency:3:4 878:0:4:1.00:1.00:0.43:0.60), (Amoebic.colitis:3:4878:0:4:1.00:1.00:0.43:0.60), (Arterial.injury:3: 4878:0:4:1.00:1.00:0.43:0.60), (Borderline.serous.tumour.of.ovary:3:4878:0:4:1.00:1.00:0.43:0.6 0), (Cyclitis:3:4878:0:4:1.00:1.00:0.43:0.60), (Diffuse.mesangial.sclerosis:3:4878:0:4:1.00:1.00:0. 43:0.60), (Duodenal.stenosis:3:4878:0:4:1.00:1.00:0.43:0.60), (Ectopic.pregnancy.termination:3:4 878:0:4:1.00:1.00:0.43:0.60), (Extragonadal.primary.malignant.teratoma:3:4878:0:4:1.00:1.00:0. 43:0.60), (Lymphadenitis.bacterial:3:4878:1:3:1.00:0.75:0.50:0.60), (Mucocutaneous.leishmaniasi s:3:4878:0:4:1.00:1.00:0.43:0.60), (Ovarian.germ.cell.teratoma.stage.I:3:4878:0:4:1.00:1.00:0.43: 0.60), (Ovarian.germ.cell.tumour:3:4878:0:4:1.00:1.00:0.43:0.60), (Pancreaticosplenectomy:3:487 8:0:4:1.00:1.00:0.43:0.60), (Releasing.factor.agonist.therapy:3:4878:0:4:1.00:1.00:0.43:0.60), (Re tinal.pigment.epitheliopathy:3:4878:0:4:1.00:1.00:0.43:0.60), (Small.intestinal.stenosis:3:4878:0: 4:1.00:1.00:0.43:0.60), (Trisomy.13:3:4878:0:4:1.00:1.00:0.43:0.60), (Androgen.insensitivity.syn drome:2:4879:0:4:1.00:1.00:0.33:0.50), (Antinuclear.antibody.increased:2:4879:0:4:1.00:1.00:0.3 3:0.50), (Bile.output.decreased:2:4879:0:4:1.00:1.00:0.33:0.50), (Bladder.cancer.stage.I,.with.can cer.in.situ:2:4879:0:4:1.00:1.00:0.33:0.50), (Blood.immunoglobulin.A.abnormal:2:4879:0:4:1.00: 1.00:0.33:0.50), (Clear.cell.sarcoma.of.the.kidney:2:4879:0:4:1.00:1.00:0.33:0.50), (Craniopharyn gioma.benign:2:4879:0:4:1.00:1.00:0.33:0.50), (Cytomegalovirus.duodenitis:2:4879:0:4:1.00:1.0 0:0.33:0.50), (Drug.clearance:2:4879:0:4:1.00:1.00:0.33:0.50), (Eccrine.carcinoma:2:4879:0:4:1.0 0:1.00:0.33:0.50), (Endocarditis.fibroplastica:2:4879:0:4:1.00:1.00:0.33:0.50), (Exposure.via.bloo d:2:4879:0:4:1.00:1.00:0.33:0.50), (Flavivirus.test.positive:2:4879:0:4:1.00:1.00:0.33:0.50), (Ham artoma.vascular:2:4879:0:4:1.00:1.00:0.33:0.50), (Hepatitis.B.e.antibody.positive:2:4879:0:4:1.0 0:1.00:0.33:0.50), (Herpes.simplex.virus.conjunctivitis.neonatal:2:4879:0:4:1.00:1.00:0.33:0.50), (Injection.site.induration:2:4879:0:4:1.00:1.00:0.33:0.50), (Lenegre's.disease:2:4879:0:4:1.00:1.00:0.33:0.50), (Listeria.sepsis:2:4879:0:4:1.00:1.00:0.33:0.50), (Medullary.carcinoma.of.breast:2:48 79:0:4:1.00:1.00:0.33:0.50), (Ocular.cancer.metastatic:2:4879:0:4:1.00:1.00:0.33:0.50), (Oesopha geal.adenocarcinoma.stage.IV:2:4879:0:4:1.00:1.00:0.33:0.50), (Oesophageal.ulcer.perforation:2: 4879:0:4:1.00:1.00:0.33:0.50), (Oesophagogastric.fundoplasty:2:4879:0:4:1.00:1.00:0.33:0.50), (P aget-Schroetter.syndrome:2:4879:0:4:1.00:1.00:0.33:0.50), (Paracoccidioides.infection:2:4879:0:4:1.0 0:1.00:0.33:0.50), (Plasma.exchange:2:4879:0:4:1.00:1.00:0.33:0.50), (Polymenorrhagia:2:4879:0: 4:1.00:1.00:0.33:0.50), (Sclerodactylia:2:4879:0:4:1.00:1.00:0.33:0.50), (Skin.striae:2:4879:0:4:1 0.00:1.00:0.33:0.50), (Skin.warm:2:4879:0:4:1.00:1.00:0.33:0.50), (Tonsil.cancer.metastatic:2:487 9:0:4:1.00:1.00:0.33:0.50), (Viral.sepsis:2:4879:0:4:1.00:1.00:0.33:0.50), (Viral.vasculitis:2:4879: 0:4:1.00:1.00:0.33:0.50), (Adjusted.calcium:1:4880:0:4:1.00:1.00:0.20:0.33), (Anal.spasm:1:4880: 0:4:1.00:1.00:0.20:0.33), (Antipsychotic.drug.level.increased:1:4880:0:4:1.00:1.00:0.20:0.33), (B acterial.pericarditis:1:4880:0:4:1.00:1.00:0.20:0.33), (Benign.respiratory.tract.neoplasm:1:4880:0:4:1.00:1.00:0.20:0.33), (Bladder.adenocarcinoma.recurrent:1:4880:0:4:1.00:1.00:0.20:0.33), (Blo od.follicle.stimulating.hormone.abnormal:1:4880:0:4:1.00:1.00:0.20:0.33), (Calculus.urethral:1:4 880:0:4:1.00:1.00:0.20:0.33), (Cardiac.myxoma:1:4880:0:4:1.00:1.00:0.20:0.33), (Chemical.bum: 1:4880:0:4:1.00:1.00:0.20:0.33), (Choking.sensation:1:4880:0:4:1.00:1.00:0.20:0.33), (Congenital.endocrine.anomaly:1:4880:0:4:1.00:1.00:0.20:0.33), (Congenital.scoliosis:1:4880:0:4:1.00:1.00: 0.20:0.33), (Constricted.affect:1:4880:0:4:1.00:1.00:0.20:0.33), (Corneal.lesion:1:4880:0:4:1.00:1. 00:0.20:0.33), (Diabetes.mellitus. malnutrition-related:1:4880:0:4:1.00:1.00:0.20:0.33), (Disorder.of.orbit:1:4880:0:4:1.00:1.00:0.20:0.33), (Dust. inhalation.pneumopathy:1:4880:0:4:1.00:1.00:0.20:0.33), (Ectropion:1:4880:0:4:1.00:1.00:0.20:0 0.33), (Elbow.operation:1:4880:0:4:1.00:1.00:0.20:0.33), (Endovenous.ablation:1:4880:0:4:1.00:1. 00:0.20:0.33), (Ephelides:1:4880:0:4:1.00:1.00:0.20:0.33), (Extragonadal.primary.non-seminoma:1:4880:0:4:1.00:1.00:0.20:0.33), (Fallopian.tube.cancer.stage.IV:1:4880:0:4:1.00:1.00: 0.20:0.33), (Fallopian.tube.neoplasm:1:4880:0:4:1.00:1.00:0.20:0.33), (Fascial.operation:1:4880:0: 4:1.00:1.00:0.20:0.33), (Fixed.eruption:1:4880:0:4:1.00:1.00:0.20:0.33), (Heart.block.congenital: 1:4880:0:4:1.00:1.00:0.20:0.33), (Hereditary.hypophosphataemic.rickets:1:4880:0:4:1.00:1.00:0. 20:0.33), (Hyperinsulinism.syndrome:1:4880:0:4:1.00:1.00:0.20:0.33), (Hyperoxaluria:1:4880:0:4: 1.00:1.00:0.20:0.33), (Hypertrophic.osteoarthropathy:1:4880:0:4:1.00:1.00:0.20:0.33), (Iliac.arter y.occlusion:1:4880:0:4:1.00:1.00:0.20:0.33), (Immune-mediated.adverse.reaction:1:4880:0:4:1.00:1.00:0.20:0.33), (Jealous.delusion:1:4880:0:4:1.00:1.0 0:0.20:0.33), (Keratoacanthoma:1:4880:0:4:1.00:1.00:0.20:0.33), (Left.atrial.dilatation:1:4880:0:4: 1.00:1.00:0.20:0.33), (Legionella.test:1:4880:0:4:1.00:1.00:0.20:0.33), (Lewis-Sumner.syndrome:1:4880:0:4:1.00:1.00:0.20:0.33), (Lhermitte's.sign:1:4880:0:4:1.00:1.00:0.20:0 0.33), (Lip.pruritus:1:4880:0:4:1.00:1.00:0.20:0.33), (Necrotising.panniculitis:1:4880:0:4:1.00:1.00: 0.20:0.33), (Neonatal.alloimmune.thrombocytopenia:1:4880:0:4:1.00:1.00:0.20:0.33), (Neonatal. disorder:1:4880:0:4:1.00:1.00:0.20:0.33), (Nephritis.radiation:1:4880:0:4:1.00:1.00:0.20:0.33), (N umb.chin.syndrome:1:4880:0:4:1.00:1.00:0.20:0.33), (Oesophagectomy:1:4880:0:4:1.00:1.00:0.2 0:0.33), (Oropharyngeal.neoplasm:1:4880:0:4:1.00:1.00:0.20:0.33), (Pancoast's.syndrome:1:4880: 0:4:1.00:1.00:0.20:0.33), (Penile.haemorrhage:1:4880:0:4:1.00:1.00:0.20:0.33), (Peritoneal.lavage: 1:4880:0:4:1.00:1.00:0.20:0.33), (PHACES.syndrome:1:4880:0:4:1.00:1.00:0.20:0.33), (Polysero sitis:1:4880:0:4:1.00:1.00:0.20:0.33), (Portal.shunt:1:4880:0:4:1.00:1.00:0.20:0.33), (Precursor.T-lymphoblastic.lymphoma/leukaemia.stage.II:1:4880:0:4:1.00:1.00:0.20:0.33), (Proteus.syndrome: 1:4880:0:4:1.00:1.00:0.20:0.33), (Pulmonary.tumour.thrombotic.microangiopathy:1:4880:0:4:1.0 0:1.00:0.20:0.33), (Pulmonary.valve.incompetence:1:4880:0:4:1.00:1.00:0.20:0.33), (Renal.artery. dissection:1:4880:0:4:1.00:1.00:0.20:0.33), (Retinal.artery.thrombosis:1:4880:0:4:1.00:1.00:0.20: 0.33), (Retinal.depigmentation:1:4880:0:4:1.00:1.00:0.20:0.33), (Retrograde.menstruation:1:4880: 0:4:1.00:1.00:0.20:0.33), (Roseolovirus.test.positive:1:4880:0:4:1.00:1.00:0.20:0.33), (Spermatog enesis.abnormal:1:4880:0:4:1.00:1.00:0.20:0.33), (Subretinal.fibrosis:1:4880:0:4:1.00:1.00:0.20:0 0.33), (Teeth.brittle:1:4880:0:4:1.00:1.00:0.20:0.33), (Trichophytic.granuloma:1:4880:0:4:1.00:1.0 0:0.20:0.33), (Verbal.abuse:1:4880:0:4:1.00:1.00:0.20:0.33), (Vertigo.CNS.origin:1:4880:0:4:1.00: 1.00:0.20:0.33), (Visual.pathway.disorder:1:4880:0:4:1.00:1.00:0.20:0.33), (Voyeurism:1:4880:0: 4:1.00:1.00:0.20:0.33), (17-alpha-hydroxylase.deficiency:0:4880:0:5:1.00:#NUM!:0.00:#NUM!), (Abscess.management:0:4880:0: 5:1.00:#NUM!:0.00:#NUM!), (Administration.site.hypersensitivity:0:4880:0:5:1.00:#NUM!:0.00: #NUM!), (Adrenal.atrophy:0:4880:0:5:1.00:#NUM!:0.00:#NUM!), (Adrenal.insufficiency.neonat al:0:4880:0:5:1.00:#NUM!:0.00:#NUM!), (Agitation.neonatal:0:4880:0:5:1.00:#NUM!:0.00:#NU M!), (Albumin.urine.present:0:4880:0:5:1.00:#NUM!:0.00:#NUM!), (Alveolar.oxygen.partial.pre ssure.decreased:0:4880:0:5:1.00:#NUM!:0.00:#NUM!), (Amaurosis:0:4880:0:5:1.00:#NUM!:0.0 0:#NUM!), (Amoebic.brain.abscess:0:4880:0:5:1.00:#NUM!:0.00:#NUM!), (Anal.cancer.stage.III: 0:4880:0:5:1.00:#NUM!:0.00:#NUM!), (Anal.lesion.excision:0:4880:0:5:1.00:#NUM!:0.00:#NU M!), (Anal.sphincterotomy:0:4880:0:5:1.00:#NUM!:0.00:#NUM!), (Angiogram.abnormal:0:4880: 0:5:1.00:#NUM!:0.00:#NUM!), (Anti-cyclic.citrullinated.peptide.antibody:0:4880:0:5:1.00:#NUM!:0.00:#NUM!), (Application.site.sw elling:0:4880:0:5:1.00:#NUM!:0.00:#NUM!), (Arterial.haemorrhage:0:4880:0:5:1.00:#NUM!:0.0 0:#NUM!), (Arteriovenous.fistula.site.haemorrhage:0:4880:0:5:1.00:#NUM!:0.00:#NUM!), (Arth ritis.climacteric:0:4880:0:5:1.00:#NUM!:0.00:#NUM!), (Atrioventricular.dissociation:0:4880:0:5 0.1.00:#NUM!:0.00:#NUM!), (Bacterial.rhinitis:0:4880:0:5:1.00:#NUM!:0.00:#NUM!), (Bacterial. test.negative:0:4880:0:5:1.00:#NUM!:0.00:#NUM!), (Balloon-occluded.retrograde.transvenous.obliteration:0:4880:0:5:1.00:#NUM!:0.00:#NUM!), (Barium.do uble.contrast:0:4880:0:5:1.00:#NUM!:0.00:#NUM!), (Bicuspid.aortic.valve:0:4880:0:5:1.00:#NU M!:0.00:#NUM!), (Bilirubin.excretion.disorder:0:4880:0:5:1.00:#NUM!:0.00:#NUM!), (Blindnes s.transient:0:4880:0:5:1.00:#NUM!:0.00:#NUM!), (Blood.catecholamines.abnormal:0:4880:0:5:1 0.00:#NUM!:0.00:#NUM!), (Blood.luteinising.hormone.abnormal:0:4880:0:5:1.00:#NUM!:0.00:#NUM!), (Blood.pressure.ambulatory.increased:0:4880:0:5:1.00:#NUM!:0.00:#NUM!), (Brain.em pyema:0:4880:0:5:1.00:#NUM!:0.00:#NUM!), (Buried.penis.syndrome:0:4880:0:5:1.00:#NUM!: 0.00:#NUM!), (Cardiac.index.abnormal:0:4880:0:5:1.00:#NUM!:0.00:#NUM!), (Cellulitis.pharyn geal:0:4880:0:5:1.00:#NUM!:0.00:#NUM!), (Central.auditory.processing.disorder:0:4880:0:5:1.0 0:#NUM!:0.00:#NUM!), (Cerebrosclerosis:0:4880:0:5:1.00:#NUM!:0.00:#NUM!), (Choroidal.ha emorrhage:0:4880:0:5:1.00:#NUM!:0.00:#NUM!), (Chronic.throat.clearing:0:4880:0:5:1.00:#NU M!:0.00:#NUM!), (Coital.bleeding:0:4880:0:5:1.00:#NUM!:0.00:#NUM!), (Colporrhaphy:0:4880: 0:5:1.00:#NUM!:0.00:#NUM!), (Computerised.tomogram.intestine:0:4880:0:5:1.00:#NUM!:0.0 0:#NUM!), (Congenital.pneumonia:0:4880:0:5:1.00:#NUM!:0.00:#NUM!), (CSF.test.abnormal:0: 4880:0:5:1.00:#NUM!:0.00:#NUM!), (Cystitis.radiation:0:4880:0:5:1.00:#NUM!:0.00:#NUM!), (Cystoprostatectomy:0:4880:0:5:1.00:#NUM!:0.00:#NUM!), (Dacryostenosis.congenital:0:4880:0: 5:1.00:#NUM!:0.00:#NUM!), (Dengue.fever:0:4880:0:5:1.00:#NUM!:0.00:#NUM!), (Depersona lisation/derealisation.disorder:0:4880:0:5:1.00:#NUM!:0.00:#NUM!), (Derealisation:0:4880:0:5: 1.00:#NUM!:0.00:#NUM!), (Dilated.pores:0:4880:0:5:1.00:#NUM!:0.00:#NUM!), (Double.ureter: 0:4880:0:5:1.00:#NUM!:0.00:#NUM!), (Ear.infection.staphylococcal:0:4880:0:5:1.00:#NUM!:0. 00:#NUM!), (Enamel.anomaly:0:4880:0:5:1.00:#NUM!:0.00:#NUM!), (Endoscopy.small.intestin e:0:4880:0:5:1.00:#NUM!:0.00:#NUM!), (Endotoxic.shock:0:4880:0:5:1.00:#NUM!:0.00:#NUM !), (Eosinophilic.myocarditis:0:4880:0:5:1.00:#NUM!:0.00:#NUM!), (Epidural.haemorrhage:0:48 80:0:5:1.00:#NUM!:0.00:#NUM!), (Episiotomy:0:4880:0:5:1.00:#NUM!:0.00:#NUM!), (Excessi ve.exercise:0:4880:0:5:1.00:#NUM!:0.00:#NUM!), (Exercise.test.abnormal:0:4880:0:5:1.00:#NU M!:0.00:#NUM!), (Exfoliation.syndrome:0:4880:0:5:1.00:#NUM!:0.00:#NUM!), (Extra.dose.ad ministered:0:4880:0:5:1.00:#NUM!:0.00:#NUM!), (Factor.V.inhibition:0:4880:0:5:1.00:#NUM!: 0.00:#NUM!), (Factor.X.deficiency:0:4880:0:5:1.00:#NUM!:0.00:#NUM!), (Fasciolopsiasis:0:48 80:0:5:1.00:#NUM!:0.00:#NUM!), (Fibrinolysis.abnormal:0:4880:0:5:1.00:#NUM!:0.00:#NUM!), (Flail.chest:0:4880:0:5:1.00:#NUM!:0.00:#NUM!), (Flat.anterior.chamber.of.eye:0:4880:0:5:1. 00:#NUM!:0.00:#NUM!), (Foetal.heart.rate.increased:0:4880:0:5:1.00:#NUM!:0.00:#NUM!), (Fo od.contamination:0:4880:0:5:1.00:#NUM!:0.00:#NUM!), (Gardnerella.infection:0:4880:0:5:1.00: #NUM!:0.00:#NUM!), (Gastritis.herpes:0:4880:0:5:1.00:#NUM!:0.00:#NUM!), (Gene.sequencin g:0:4880:0:5:1.00:#NUM!:0.00:#NUM!), (Giant.cell.epulis:0:4880:0:5:1.00:#NUM!:0.00:#NUM !), (Gingival.inflammation:0:4880:0:5:1.00:#NUM!:0.00:#NUM!), (Granulocyte.count:0:4880:0:5: 1.00:#NUM!:0.00:#NUM!), (Haemophilia.B.without.inhibitors:0:4880:0:5:1.00:#NUM!:0.00:#N UM!), (Hepatic.amyloidosis:0:4880:0:5:1.00:#NUM!:0.00:#NUM!), (Hepatic.haematoma:0:4880: 0:5:1.00:#NUM!:0.00:#NUM!), (Hepatitis.G:0:4880:0:5:1.00:#NUM!:0.00:#NUM!), (Hepatobilia ry.neoplasm:0:4880:0:5:1.00:#NUM!:0.00:#NUM!), (Hepatorenal.failure:0:4880:0:5:1.00:#NUM !:0.00:#NUM!), (Hygroma.colli:0:4880:0:5:1.00:#NUM!:0.00:#NUM!), (Hyposmia:0:4880:0:5:1. 00:#NUM!:0.00:#NUM!), (Impaired.insulin.secretion:0:4880:0:5:1.00:#NUM!:0.00:#NUM!), (Im paired.reasoning:0:4880:0:5:1.00:#NUM!:0.00:#NUM!), (Incisional.hernia:0:4880:0:5:1.00:#NU M!:0.00:#NUM!), (Increased.viscosity.of.nasal.secretion:0:4880:0:5:1.00:#NUM!:0.00:#NUM!), (Injectable.contraception:0:4880:0:5:1.00:#NUM!:0.00:#NUM!), (Insertion.of.ambulatory.periton eal.catheter:0:4880:0:5:1.00:#NUM!:0.00:#NUM!), (Intestinal.scarring:0:4880:0:5:1.00:#NUM!: 0.00:#NUM!), (Intra-uterine.contraceptive.device.removal:0:4880:0:5:1.00:#NUM!:0.00:#NUM!), (Job.dissatisfaction: 0:4880:0:5:1.00:#NUM!:0.00:#NUM!), (Judgement.impaired:0:4880:0:5:1.00:#NUM!:0.00:#NU M!), (Keratorhexis:0:4880:0:5:1.00:#NUM!:0.00:#NUM!), (Laryngeal.discomfort:0:4880:0:5:1.0 0:#NUM!:0.00:#NUM!), (LDL/HDL.ratio.increased:0:4880:0:5:1.00:#NUM!:0.00:#NUM!), (Leu kaemic.infiltration.extramedullary:0:4880:0:5:1.00:#NUM!:0.00:#NUM!), (Limb.traumatic.ampu tation:0:4880:0:5:1.00:#NUM!:0.00:#NUM!), (Lipoedema:0:4880:0:5:1.00:#NUM!:0.00:#NUM!), (Local.reaction:0:4880:0:5:1.00:#NUM!:0.00:#NUM!), (Ludwig.angina:0:4880:0:5:1.00:#NUM !:0.00:#NUM!), (Macrocephaly:0:4880:0:5:1.00:#NUM!:0.00:#NUM!), (Male.genital.examinatio n.abnormal:0:4880:0:5:1.00:#NUM!:0.00:#NUM!), (Malocclusion:0:4880:0:5:1.00:#NUM!:0.00: #NUM!), (Maternal.condition.affecting.foetus:0:4880:0:5:1.00:#NUM!:0.00:#NUM!), (Maternal. distress.during.labour:0:4880:0:5:1.00:#NUM!:0.00:#NUM!), (Medical.device.site.abscess:0:488 0:0:5:1.00:#NUM!:0.00:#NUM!), (Metastases.to.peripheral.vascular.system:0:4880:0:5:1.00:#N UM!:0.00:#NUM!), (Mevalonic.aciduria:0:4880:0:5:1.00:#NUM!:0.00:#NUM!), (Mumps:0:4880: 0:5:1.00:#NUM!:0.00:#NUM!), (Musculoskeletal.deformity:0:4880:0:5:1.00:#NUM!:0.00:#NU M!), (Musculoskeletal.injury:0:4880:0:5:1.00:#NUM!:0.00:#NUM!), (Mycoplasma.genitalium.inf ection:0:4880:0:5:1.00:#NUM!:0.00:#NUM!), (Mycoplasma.test.positive:0:4880:0:5:1.00:#NUM !:0.00:#NUM!), (Myocarditis.post.infection:0:4880:0:5:1.00:#NUM!:0.00:#NUM!), (Natural.kille r.T.cell.count:0:4880:0:5:1.00:#NUM!:0.00:#NUM!), (Neonatal.tachycardia:0:4880:0:5:1.00:#N UM!:0.00:#NUM!), (Neuritic.plaques:0:4880:0:5:1.00:#NUM!:0.00:#NUM!), (Neurotrophic.kerat opathy:0:4880:0:5:1.00:#NUM!:0.00:#NUM!), (Oesophageal.mucosa.erythema:0:4880:0:5:1.00: #NUM!:0.00:#NUM!), (Oestradiol.increased:0:4880:0:5:1.00:#NUM!:0.00:#NUM!), (Onychomal acia:0:4880:0:5:1.00:#NUM!:0.00:#NUM!), (Oocyte.donor:0:4880:0:5:1.00:#NUM!:0.00:#NUM !), (Orbital.apex.syndrome:0:4880:0:5:1.00:#NUM!:0.00:#NUM!), (Ovarian.germ.cell.cancer.stag e.II:0:4880:0:5:1.00:#NUM!:0.00:#NUM!), (Parasite.allergy:0:4880:0:5:1.00:#NUM!:0.00:#NU M!), (Pelvic.floor.repair:0:4880:0:5:1.00:#NUM!:0.00:#NUM!), (Penile.wart:0:4880:0:5:1.00:#N UM!:0.00:#NUM!), (Periorbital.disorder:0:4880:0:5:1.00:#NUM!:0.00:#NUM!), (Peripheral.T-cell.lymphoma.unspecified.stage.III:0:4880:0:5:1.00:#NUM!:0.00:#NUM!), (Pharyngeal.mucosa. atrophy:0:4880:0:5:1.00:#NUM!:0.00:#NUM!), (Pigmentation.lip:0:4880:0:5:1.00:#NUM!:0.00: #NUM!), (Plasminogen:0:4880:0:5:1.00:#NUM!:0.00:#NUM!), (Plastic.surgery.to.the.face:0:488 0:0:5:1.00:#NUM!:0.00:#NUM!), (Polycythaemia.neonatorum:0:4880:0:5:1.00:#NUM!:0.00:#N UM!), (Polymorphic.eruption.of.pregnancy:0:4880:0:5:1.00:#NUM!:0.00:#NUM!), (Portal.vein.o cclusion:0:4880:0:5:1.00:#NUM!:0.00:#NUM!), (Portal.vein.pressure.increased:0:4880:0:5:1.00: #NUM!:0.00:#NUM!), (Post.cardiac.arrest.syndrome:0:4880:0:5:1.00:#NUM!:0.00:#NUM!), (Pro duct.compounding.quality.issue:0:4880:0:5:1.00:#NUM!:0.00:#NUM!), (Product.contamination. chemical:0:4880:0:5:1.00:#NUM!:0.00:#NUM!), (Prophylaxis.against.psychomotor.agitation:0:4 880:0:5:1.00:#NUM!:0.00:#NUM!), (Rabies.immunisation:0:4880:0:5:1.00:#NUM!:0.00:#NUM!), (Rectal.tenesmus:0:4880:0:5:1.00:#NUM!:0.00:#NUM!), (Red.blood.cell.sedimentation.rate.ab normal:0:4880:0:5:1.00:#NUM!:0.00:#NUM!), (Respiratory.disorder.neonatal:0:4880:0:5:1.00:#NUM!:0.00:#NUM!), (Respiratory.papilloma:0:4880:0:5:1.00:#NUM!:0.00:#NUM!), (Right.atrial.dilatation:0:4880:0:5:1.00:#NUM!:0.00:#NUM!), (Right.ventricular.systolic.pressure:0:4880:0:5: 1.00:#NUM!:0.00:#NUM!), (Sacralisation:0:4880:0:5:1.00:#NUM!:0.00:#NUM!), (Salivary.glan d.cancer.stage.0:0:4880:0:5:1.00:#NUM!:0.00:#NUM!), (Salivary.gland.enlargement:0:4880:0:5: 1.00:#NUM!:0.00:#NUM!), (Salmonella.bacteraemia:0:4880:0:5:1.00:#NUM!:0.00:#NUM!), (Scr otal.inflammation:0:4880:0:5:1.00:#NUM!:0.00:#NUM!), (Serology.positive:0:4880:0:5:1.00:#N UM!:0.00:#NUM!), (Sphenoid.sinus.operation:0:4880:0:5:1.00:#NUM!:0.00:#NUM!), (Spinal.co rd. ischaemia:0:4880:0:5:1.00:#NUM!:0.00:#NUM!), (Spinal.meningioma.benign:0:4880:0:5:1.00: #NUM!:0.00:#NUM!), (Stag.horn.calculus:0:4880:0:5:1.00:#NUM!:0.00:#NUM!), (Staphylococ cal.impetigo:0:4880:0:5:1.00:#NUM!:0.00:#NUM!), (Stoma.complication:0:4880:0:5:1.00:#NU M!:0.00:#NUM!), (Syphilitic.endocarditis.of.heart.valve:0:4880:0:5:1.00:#NUM!:0.00:#NUM!), (Tattoo:0:4880:0:5:1.00:#NUM!:0.00:#NUM!), (Theft:0:4880:0:5:1.00:#NUM!:0.00:#NUM!), (Th erapeutic.nerve.ablation:0:4880:0:5:1.00:#NUM!:0.00:#NUM!), (Therapeutic.response.changed:0: 4880:0:5:1.00:#NUM!:0.00:#NUM!), (Thought.withdrawal:0:4880:0:5:1.00:#NUM!:0.00:#NU M!), (Thyroiditis.acute:0:4880:0:5:1.00:#NUM!:0.00:#NUM!), (Thyroxine.free.increased:0:4880: 0:5:1.00:#NUM!:0.00:#NUM!), (Tongue.carcinoma.stage.IV:0:4880:0:5:1.00:#NUM!:0.00:#NU M!), (Tracheostomy.tube.removal:0:4880:0:5:1.00:#NUM!:0.00:#NUM!), (Transurethral.resectio n.syndrome:0:4880:0:5:1.00:#NUM!:0.00:#NUM!), (Traumatic.delivery:0:4880:0:5:1.00:#NUM!: 0.00:#NUM!), (Ultrasound.uterus:0:4880:0:5:1.00:#NUM!:0.00:#NUM!), (Urinary.bladder.sarco ma:0:4880:0:5:1.00:#NUM!:0.00:#NUM!), (Vaginal.cellulitis:0:4880:0:5:1.00:#NUM!:0.00:#NU M!), (Vaginal.dysplasia:0:4880:0:5:1.00:#NUM!:0.00:#NUM!), (Vaginal.pH.increased:0:4880:0: 5:1.00:#NUM!:0.00:#NUM!), (Vascular.resistance.pulmonary.decreased:0:4880:0:5:1.00:#NUM!: 0.00:#NUM!), (Vascular.resistance.systemic:0:4880:0:5:1.00:#NUM!:0.00:#NUM!), (Viral.rhinit is:0:4880:0:5:1.00:#NUM!:0.00:#NUM!), (Volume.blood:0:4880:0:5:1.00:#NUM!:0.00:#NUM!), (Wean.from.ventilator:0:4880:0:5:1.00:#NUM!:0.00:#NUM!), (Wemicke-Korsakoff.syndrome:0:4880:0:5:1.00:#NUM!:0.00:#NUM!), (Wound.infection.viral:0:4880:0:5:1 0.00:#NUM!:0.00:#NUM!), (Xeroderma.pigmentosum:0:4880:0:5:1.00:#NUM!:0.00:#NUM!), (Bo vine.tuberculosis:11:4869:0:5:1.00:1.00:0.69:0.81), (HIV.infection.WHO.clinical.stage.II:11:486 9:0:5:1.00:1.00:0.69:0.81), (Angioimmunoblastic.T-cell.lymphoma.refractory:10:4870:0:5:1.00:1.00:0.67:0.80), (Angioimmunoblastic.T-cell.lymphoma.stage.III:10:4870:0:5:1.00:1.00:0.67:0.80), (Anencephaly:8:4872:0:5:1.00:1.00:0. 62:0.76), (Bone.marrow.tumour.cell.infiltration:8:4872:0:5:1.00:1.00:0.62:0.76), (Fulminant.type. 1.diabetes.mellitus:8:4872:0:5:1.00:1.00:0.62:0.76), (Parophthalmia:8:4872:0:5:1.00:1.00:0.62:0. 76), (Drug.effect.incomplete:7:4873:0:5:1.00:1.00:0.58:0.74), (Hodgkin's.disease.mixed.cellularit y.refractory:7:4873:0:5:1.00:1.00:0.58:0.74), (Mucinous.adenocarcinoma.of.appendix:7:4873:0:5: 1.00:1.00:0.58:0.74), (Paradoxical.drug.reaction:7:4873:0:5:1.00:1.00:0.58:0.74), (HLA.marker.s tudy.positive:6:4874:0:5:1.00:1.00:0.55:0.71), (Ovarian.germ.cell.tumour.mixed:6:4874:0:5:1.00: 1.00:0.55:0.71), (Pulmonary.renal.syndrome:6:4874:0:5:1.00:1.00:0.55:0.71), (Trisomy.12:6:4874: 0:5:1.00:1.00:0.55:0.71), (Extragonadal.primary.embryonal.carcinoma:5:4875:0:5:1.00:1.00:0.50:0.67), (Superior.mesenteric.artery.syndrome:5:4875:0:5:1.00:1.00:0.50:0.67), (T-lymphocyte.count.decreased:5:4875:0:5:1.00:1.00:0.50:0.67), (Testicular.yolk.sac.tumour.stage.III: 5:4875:0:5:1.00:1.00:0.50:0.67), (Tracheobronchial.stent.insertion:5:4875:0:5:1.00:1.00:0.50:0. 67), (Anal.cancer.recurrent:4:4876:0:5:1.00:1.00:0.44:0.62), (Hodgkin's.disease.mixed.cellularity. stage.IV:4:4876:0:5:1.00:1.00:0.44:0.62), (Idiopathic.partial.epilepsy:4:4876:0:5:1.00:1.00:0.44:0 0.62), (Mutagenic.effect:4:4876:0:5:1.00:1.00:0.44:0.62), (Ovarian.germ.cell.cancer:4:4876:0:5:1.0 0:1.00:0.44:0.62), (Tissue.adhesion.prophylaxis:4:4876:0:5:1.00:1.00:0.44:0.62), (Central.nervous.system.haemorrhage:3:4877:0:5:1.00:1.00:0.38:0.55), (Congenital.uterine.anomaly:3:4877:0:5:1. 00:1.00:0.38:0.55), (Encephalitis.enteroviral:3:4877:0:5:1.00:1.00:0.38:0.55), (Haemoglobin.S.inc reased:3:4877:0:5:1.00:1.00:0.38:0.55), (Iris.melanoma:3:4877:0:5:1.00:1.00:0.38:0.55), (Meningi tis.histoplasma:3:4877:1:4:1.00:0.75:0.43:0.55), (Metastases.to.penis:3:4877:0:5:1.00:1.00:0.38:0 0.55), (Primary.mediastinal.large.B-cell.lymphoma.stage.I:3:4877:0:5:1.00:1.00:0.38:0.55), (Purtscher.retinopathy:3:4877:0:5:1.00:1. 00:0.38:0.55), (Tuberculosis.ureter:3:4877:1:4:1.00:0.75:0.43:0.55), (Urinary.tract.carcinoma.in.si tu:3:4877:0:5:1.00:1.00:0.38:0.55), (Abdominal.injury:2:4878:0:5:1.00:1.00:0.29:0.44), (Acne.ful minans:2:4878:0:5:1.00:1.00:0.29:0.44), (Anti-GAD. antibody:2:4878:0:5:1.00:1.00:0.29:0.44), (Congenital.thrombocytopenia:2:4878:0:5:1.00:1 0.00:0.29:0.44), (Endocardial.disease:2:4878:0:5:1.00:1.00:0.29:0.44), (Exposure.to.unspecified.ag ent:2:4878:0:5:1.00:1.00:0.29:0.44), (Herpes.gestationis:2:4878:0:5:1.00:1.00:0.29:0.44), (Human.chorionic.gonadotropin:2:4878:0:5:1.00:1.00:0.29:0.44), (Hysteroscopy:2:4878:0:5:1.00:1.00:0.2 9:0.44), (Injection.site.papule:2:4878:0:5:1.00:1.00:0.29:0.44), (Klippel-Trenaunay.syndrome:2:4878:0:5:1.00:1.00:0.29:0.44), (Lymphangiectasia.intestinal:2:4878:0:5:1 0.00:1.00:0.29:0.44), (Non-Hodgkin's.lymphoma.unspecified.histology.aggressive.refractory:2:4878:0:5:1.00:1.00:0.29:0.44), (Oesophageal.operation:2:4878:0:5:1.00:1.00:0.29:0.44), (pH.urine.increased:2:4878:0:5:1.00:1. 00:0.29:0.44), (Retinal.infarction:2:4878:0:5:1.00:1.00:0.29:0.44), (Scleral.disorder:2:4878:0:5:1. 00:1.00:0.29:0.44), (T-cell.lymphoma.stage.I:2:4878:0:5:1.00:1.00:0.29:0.44), (Total.adrenalectomy:2:4878:0:5:1.00:1.0 0:0.29:0.44), (Urethral.abscess:2:4878:0:5:1.00:1.00:0.29:0.44), (Urethroscopy:2:4878:0:5:1.00:1. 00:0.29:0.44), (Vascular.resistance.pulmonary.increased:2:4878:0:5:1.00:1.00:0.29:0.44), (Viscer al.venous.thrombosis:2:4878:0:5:1.00:1.00:0.29:0.44), (Vocal.cord.leukoplakia:2:4878:0:5:1.00:1 0.00:0.29:0.44), (Vomiting.projectile:2:4878:0:5:1.00:1.00:0.29:0.44), (Anal.cancer.stage.0:1:4879: 0:5:1.00:1.00:0.17:0.29), (Aortic.valve.disease.mixed:1:4879:0:5:1.00:1.00:0.17:0.29), (Applicati on.site.perspiration:1:4879:0:5:1.00:1.00:0.17:0.29), (Benign.lung.neoplasm:1:4879:0:5:1.00:1.00:0.17:0.29), (Breast.reconstruction:1:4879:0:5:1.00:1.00:0.17:0.29), (Carcinoembryonic.antigen.in creased:1:4879:0:5:1.00:1.00:0.17:0.29), (Cardiac.septal.defect.residual.shunt:1:4879:0:5:1.00:1. 00:0.17:0.29), (Cardiac.ventriculogram.left:1:4879:0:5:1.00:1.00:0.17:0.29), (Cerebral.arteritis:1:4 879:0:5:1.00:1.00:0.17:0.29), (Chimerism:1:4879:0:5:1.00:1.00:0.17:0.29), (Choroidal.effusion:1: 4879:0:5:1.00:1.00:0.17:0.29), (Circulatory.failure.neonatal:1:4879:0:5:1.00:1.00:0.17:0.29), (Clo stridium.test:1:4879:0:5:1.00:1.00:0.17:0.29), (Combined.pulmonary.fibrosis.and.emphysema:1:4 879:0:5:1.00:1.00:0.17:0.29), (Computerised.tomogram.thorax.abnormal:1:4879:0:5:1.00:1.00:0. 17:0.29), (Congenital.disorder.of.glycosylation:1:4879:0:5:1.00:1.00:0.17:0.29), (Congenital.dysfi brinogenaemia:1:4879:0:5:1.00:1.00:0.17:0.29), (Congenital.renal.disorder:1:4879:0:5:1.00:1.00: 0.17:0.29), (Cutaneous.sporotrichosis:1:4879:0:5:1.00:1.00:0.17:0.29), (Cutaneovisceral.angiomat osis.with.thrombocytopenia:1:4879:0:5:1.00:1.00:0.17:0.29), (Dental.impression.procedure:1:487 9:0:5:1.00:1.00:0.17:0.29), (Dermatologic.examination.abnormal:1:4879:0:5:1.00:1.00:0.17:0.29), (Drug.dose.omission:1:4879:0:5:1.00:1.00:0.17:0.29), (Enchondromatosis:1:4879:0:5:1.00:1.00: 0.17:0.29), (Endometritis.decidual:1:4879:0:5:1.00:1.00:0.17:0.29), (Enzyme.level.increased:1:48 79:0:5:1.00:1.00:0.17:0.29), (Exposure.to.body.fluid:1:4879:0:5:1.00:1.00:0.17:0.29), (Factor.XIII. Inhibition:1:4879:0:5:1.00:1.00:0.17:0.29), (Galactose.intolerance:1:4879:0:5:1.00:1.00:0.17:0.2 9), (Gastroenteritis.norovirus:1:4879:0:5:1.00:1.00:0.17:0.29), (Gene.mutation.identification.test.p ositive:1:4879:0:5:1.00:1.00:0.17:0.29), (Gulf.war.syndrome:1:4879:0:5:1.00:1.00:0.17:0.29), (Ha emodynamic.test.abnormal:1:4879:0:5:1.00:1.00:0.17:0.29), (Haemoglobin.E-thalassaemia.disease:1:4879:0:5:1.00:1.00:0.17:0.29), (Hepatic.infarction:1:4879:0:5:1.00:1.00:0. 17:0.29), (Herpes.simplex.otitis.externa:1:4879:0:5:1.00:1.00:0.17:0.29), (Iatrogenic.injury:1:487 9:0:5:1.00:1.00:0.17:0.29), (Immune.recovery.uveitis:1:4879:0:5:1.00:1.00:0.17:0.29), (Internal.fi xation.of.spine:1:4879:0:5:1.00:1.00:0.17:0.29), (Intestinal.transit.time.abnormal:1:4879:0:5:1.00: 1.00:0.17:0.29), (Lymphomatoid.papulosis:1:4879:0:5:1.00:1.00:0.17:0.29), (Lysinuric.protein.in tolerance:1:4879:0:5:1.00:1.00:0.17:0.29), (Meningoencephalitis.bacterial:1:4879:0:5:1.00:1.00:0 0.17:0.29), (Mesenteric.vascular.insufficiency:1:4879:0:5:1.00:1.00:0.17:0.29), (Norovirus.test.posi tive:1:4879:0:5:1.00:1.00:0.17:0.29), (Oesophageal.obstruction:1:4879:0:5:1.00:1.00:0.17:0.29), (Orthopaedic.examination.abnormal:1:4879:0:5:1.00:1.00:0.17:0.29), (Paget's.disease.of.nipple:1: 4879:0:5:1.00:1.00:0.17:0.29), (Parkinsonian.crisis:1:4879:0:5:1.00:1.00:0.17:0.29), (Phototherap y:1:4879:0:5:1.00:1.00:0.17:0.29), (Placenta.praevia:1:4879:0:5:1.00:1.00:0.17:0.29), (Pneumotho rax.spontaneous:1:4879:0:5:1.00:1.00:0.17:0.29), (Progestin.therapy:1:4879:0:5:1.00:1.00:0.17:0. 29), (Pseudobulbar.palsy:1:4879:0:5:1.00:1.00:0.17:0.29), (Psoas.sign:1:4879:0:5:1.00:1.00:0.17: 0.29), (Psychological.abuse:1:4879:0:5:1.00:1.00:0.17:0.29), (Pulmonary.dysmaturity.syndrome:1:4879:0:5:1.00:1.00:0.17:0.29), (Rathke's.cleft.cyst:1:4879:0:5:1.00:1.00:0.17:0.29), (Respiratory. syncytial.virus.bronchiolitis:1:4879:0:5:1.00:1.00:0.17:0.29), (Rickettsiosis:1:4879:0:5:1.00:1.00: 0.17:0.29), (Roseola:1:4879:0:5:1.00:1.00:0.17:0.29), (Somatosensory.evoked.potentials:1:4879:0: 5:1.00:1.00:0.17:0.29), (Spleen.operation:1:4879:0:5:1.00:1.00:0.17:0.29), (Splinter:1:4879:0:5:1 0.00:1.00:0.17:0.29), (Subcutaneous.emphysema:1:4879:0:5:1.00:1.00:0.17:0.29), (Thyroid.haemor rhage:1:4879:0:5:1.00:1.00:0.17:0.29), (Tooth.malformation:1:4879:0:5:1.00:1.00:0.17:0.29), (Tra nsitional.cell.carcinoma.recurrent:1:4879:0:5:1.00:1.00:0.17:0.29), (Urine.uric.acid.decreased:1:4 879:0:5:1.00:1.00:0.17:0.29), (Uvulitis:1:4879:0:5:1.00:1.00:0.17:0.29), (Venom.poisoning:1:487 9:0:5:1.00:1.00:0.17:0.29), (Vestibular.ataxia:1:4879:0:5:1.00:1.00:0.17:0.29), (Viral.myocarditis: 1:4879:0:5:1.00:1.00:0.17:0.29), (Abdominal.wound.dehiscence:0:4879:0:6:1.00:#NUM!:0.00:#NUM!), (Acquired.claw.toe:0:4879:0:6:1.00:#NUM!:0.00:#NUM!), (Acrophobia:0:4879:0:6:1.00: #NUM!:0.00:#NUM!), (Acute.motor.axonal.neuropathy:0:4879:0:6:1.00:#NUM!:0.00:#NUM!), (Adjustment.disorder.with.disturbance.of.conduct:0:4879:0:6:1.00:#NUM!:0.00:#NUM!), (Alcoh olic.pancreatitis:0:4879:0:6:1.00:#NUM!:0.00:#NUM!), (Anaesthetic.complication:0:4879:0:6:1. 00:#NUM!:0.00:#NUM!), (Anaesthetic.complication.pulmonary:0:4879:0:6:1.00:#NUM!:0.00:#NUM!), (Anginal.equivalent:0:4879:0:6:1.00:#NUM!:0.00:#NUM!), (Anterior.chamber.disorder: 0:4879:0:6:1.00:#NUM!:0.00:#NUM!), (Anti-complement.antibody:0:4879:0:6:1.00:#NUM!:0.00:#NUM!), (Aortic.injury:0:4879:0:6:1.00:#N UM!:0.00:#NUM!), (Aortic.rupture:0:4879:0:6:1.00:#NUM!:0.00:#NUM!), (Application.site.eros ion:0:4879:0:6:1.00:#NUM!:0.00:#NUM!), (Application.site.reaction:0:4879:0:6:1.00:#NUM!:0. 00:#NUM!), (Arterectomy:0:4879:0:6:1.00:#NUM!:0.00:#NUM!), (Arteriogram.abnormal:0:4879: 0:6:1.00:#NUM!:0.00:#NUM!), (Asocial.behaviour:0:4879:0:6:1.00:#NUM!:0.00:#NUM!), (Aur iculotemporal.syndrome:0:4879:0:6:1.00:#NUM!:0.00:#NUM!), (Barium. enema:0:4879:0:6:1.00: #NUM!:0.00:#NUM!), (Bilirubinuria:0:4879:0:6:1.00:#NUM!:0.00:#NUM!), (Binge.drinking:0: 4879:0:6:1.00:#NUM!:0.00:#NUM!), (Biopsy.breast.abnormal:0:4879:0:6:1.00:#NUM!:0.00:#N UM!), (Biopsy.lip:0:4879:0:6:1.00:#NUM!:0.00:#NUM!), (Biopsy.oesophagus:0:4879:0:6:1.00:#NUM!:0.00:#NUM!), (Blister.rupture:0:4879:0:6:1.00:#NUM!:0.00:#NUM!), (Blood.chloride.inc reased:0:4879:0:6:1.00:#NUM!:0.00:#NUM!), (Blood.copper.increased:0:4879:0:6:1.00:#NUM!: 0.00:#NUM!), (Blood.pressure.diastolic.abnormal:0:4879:0:6:1.00:#NUM!:0.00:#NUM!), (Blood. urea.abnormal:0:4879:0:6:1.00:#NUM!:0.00:#NUM!), (Brachiocephalic.vein.thrombosis:0:4879: 0:6:1.00:#NUM!:0.00:#NUM!), (Brachytherapy.to.skin:0:4879:0:6:1.00:#NUM!:0.00:#NUM!), (Bradycardia.foetal:0:4879:0:6:1.00:#NUM!:0.00:#NUM!), (Brain.compression:0:4879:0:6:1.00:#NUM!:0.00:#NUM!), (Breast.disorder.male:0:4879:0:6:1.00:#NUM!:0.00:#NUM!), (Brugada.syn drome:0:4879:0:6:1.00:#NUM!:0.00:#NUM!), (Bums.first.degree:0:4879:0:6:1.00:#NUM!:0.00: #NUM!), (Cardiovascular.function.test.abnormal:0:4879:0:6:1.00:#NUM!:0.00:#NUM!), (Carotid ynia:0:4879:0:6:1.00:#NUM!:0.00:#NUM!), (Catheter.site.pruritus:0:4879:0:6:1.00:#NUM!:0.00: #NUM!), (Central-alveolar.hypoventilation:0:4879:0:6:1.00:#NUM!:0.00:#NUM!), (Cerebral.artery.thrombosis:0:4 879:0:6:1.00:#NUM!:0.00:#NUM!), (Chemotherapy.cardiotoxicity.attenuation:0:4879:0:6:1.00:#NUM!:0.00:#NUM!), (Chondrosis:0:4879:0:6:1.00:#NUM!:0.00:#NUM!), (Cold.agglutinins:0:48 79:0:6:1.00:#NUM!:0.00:#NUM!), (Colitis.erosive:0:4879:0:6:1.00:#NUM!:0.00:#NUM!), (Com munication.disorder:0:4879:0:6:1.00:#NUM!:0.00:#NUM!), (Community.acquired.infection:0:48 79:0:6:1.00:#NUM!:0.00:#NUM!), (Congenital.heart.valve.disorder:0:4879:0:6:1.00:#NUM!:0.0 0:#NUM!), (Congenital.thyroid.disorder:0:4879:0:6:1.00:#NUM!:0.00:#NUM!), (Conjunctival.cy st:0:4879:0:6:1.00:#NUM!:0.00:#NUM!), (Conjunctival.ulcer:0:4879:0:6:1.00:#NUM!:0.00:#NU M!), (Coronary.artery.stent.removal:0:4879:0:6:1.00:#NUM!:0.00:#NUM!), (Coronary.ostial.sten osis:0:4879:0:6:1.00:#NUM!:0.00:#NUM!), (Corticotropin-releasing.hormone.stimulation.test:0:4879:0:6:1.00:#NUM!:0.00:#NUM!), (Corynebacterium.sep sis:0:4879:0:6:1.00:#NUM!:0.00:#NUM!), (Creatorrhoea:0:4879:0:6:1.00:#NUM!:0.00:#NUM!), (Critical.illness.polyneuropathy:0:4879:0:6:1.00:#NUM!:0.00:#NUM!), (Cycloplegic.refraction:0: 4879:0:6:1.00:#NUM!:0.00:#NUM!), (Dementia.of.the.Alzheimer's.type,.with.delirium:0:4879:0: 6:1.00:#NUM!:0.00:#NUM!), (Device. embolisation:0:4879:0:6:1.00:#NUM!:0.00:#NUM!), (Dis seminated.large.cell.lymphoma:0:4879:0:6:1.00:#NUM!:0.00:#NUM!), (Donor.site.complication: 0:4879:0:6:1.00:#NUM!:0.00:#NUM!), (Double.hit.lymphoma:0:4879:0:6:1.00:#NUM!:0.00:#N UM!), (Dural.arteriovenous.fistula:0:4879:0:6:1.00:#NUM!:0.00:#NUM!), (Electrocardiogram.Q RS. complex.prolonged:0:4879:0:6:1.00:#NUM!:0.00:#NUM!), (Electrocardiogram.ST.segment. d epression:0:4879:0:6:1.00:#NUM!:0.00:#NUM!), (Escherichia.vaginitis:0:4879:0:6:1.00:#NUM!: 0.00:#NUM!), (Exposure.via.ingestion:0:4879:0:6:1.00:#NUM!:0.00:#NUM!), (Exposure.via.tran splant:0:4879:0:6:1.00:#NUM!:0.00:#NUM!), (Fahr's.disease:0:4879:0:6:1.00:#NUM!:0.00:#NU M!), (Familial.polycythaemia:0:4879:0:6:1.00:#NUM!:0.00:#NUM!), (Fear.of.death:0:4879:0:6:1 0.00:#NUM!:0.00:#NUM!), (Flea.infestation:0:4879:0:6:1.00:#NUM!:0.00:#NUM!), (Fluid.balanc e.negative:0:4879:0:6:1.00:#NUM!:0.00:#NUM!), (Foreign.travel:0:4879:0:6:1.00:#NUM!:0.00: #NUM!), (Funguria:0:4879:0:6:1.00:#NUM!:0.00:#NUM!), (Gallbladder.cancer.stage.IV:0:4879: 0:6:1.00:#NUM!:0.00:#NUM!), (Gardnerella.test.positive:0:4879:0:6:1.00:#NUM!:0.00:#NUM!), (Gastric.ulcer.surgery:0:4879:0:6:1.00:#NUM!:0.00:#NUM!), (Gastrointestinal.ulcer.perforation: 0:4879:0:6:1.00:#NUM!:0.00:#NUM!), (Gastrorrhaphy:0:4879:0:6:1.00:#NUM!:0.00:#NUM!), (General.physical.condition.normal:0:4879:0:6:1.00:#NUM!:0.00:#NUM!), (Haemodynamic.test: 0:4879:0:6:1.00:#NUM!:0.00:#NUM!), (Hepatic.atrophy:0:4879:0:6:1.00:#NUM!:0.00:#NUM!), (Herpangina:0:4879:0:6:1.00:#NUM!:0.00:#NUM!), (Herpes.zoster.cutaneous. disseminated:0:48 79:0:6:1.00:#NUM!:0.00:#NUM!), (Holoprosencephaly:0:4879:0:6:1.00:#NUM!:0.00:#NUM!), (Hyperlipasaemia:0:4879:0:6:1.00:#NUM!:0.00:#NUM!), (Hyperosmolar.state:0:4879:0:6:1.00:#NUM!:0.00:#NUM!), (Hypertrichosis:0:4879:0:6:1.00:#NUM!:0.00:#NUM!), (Ideas.of.reference: 0:4879:0:6:1.00:#NUM!:0.00:#NUM!), (Idiopathic.neutropenia:0:4879:0:6:1.00:#NUM!:0.00:#N UM!), (Implant.site.inflammation:0:4879:0:6:1.00:#NUM!:0.00:#NUM!), (Inbom.error.of.metabo lism:0:4879:0:6:1.00:#NUM!:0.00:#NUM!), (Infusion.site.infection:0:4879:0:6:1.00:#NUM!:0.0 0:#NUM!), (Inner.ear.inflammation:0:4879:0:6:1.00:#NUM!:0.00:#NUM!), (Intestinal.mass:0:48 79:0:6:1.00:#NUM!:0.00:#NUM!), (Intraperitoneal.hyperthermic.chemotherapy:0:4879:0:6:1.00: #NUM!:0.00:#NUM!), (Intrauterine.infection:0:4879:0:6:1.00:#NUM!:0.00:#NUM!), (Irregular.sl eep.wake.rhythm.disorder:0:4879:0:6:1.00:#NUM!:0.00:#NUM!), (Jessner's.lymphocytic.infiltrat ion:0:4879:0:6:1.00:#NUM!:0.00:#NUM!), (Kleptomania:0:4879:0:6:1.00:#NUM!:0.00:#NUM!), (Labial.tie:0:4879:0:6:1.00:#NUM!:0.00:#NUM!), (Laryngeal.dyspnoea:0:4879:0:6:1.00:#NUM: 0.00:#NUM!), (Laryngeal.papilloma:0:4879:0:6:1.00:#NUM!:0.00:#NUM!), (Light.chain.analys is.increased:0:4879:0:6:1.00:#NUM!:0.00:#NUM!), (Malignant.neoplasm.of.conjunctiva:0:4879: 0:6:1.00:#NUM!:0.00:#NUM!), (Meniscus.removal:0:4879:0:6:1.00:#NUM!:0.00:#NUM!), (Mid-life.crisis:0:4879:0:6:1.00:#NUM!:0.00:#NUM!), (Middle.ear.inflammation:0:4879:0:6:1.00:#N UM!:0.00:#NUM!), (Milk-alkali.syndrome:0:4879:0:6:1.00:#NUM!:0.00:#NUM!), (Monogenic.diabetes:0:4879:0:6:1.00:#NUM!:0.00:#NUM!), (Mucolipidosis.type.II:0:4879:0:6:1.00:#NUM!:0.00:#NUM!), (Multiple.en docrine.neoplasia:0:4879:0:6:1.00:#NUM!:0.00:#NUM!), (Mumps.immunisation:0:4879:0:6:1.00: #NUM!:0.00:#NUM!), (Myeloid.leukaemia.in.remission:0:4879:0:6:1.00:#NUM!:0.00:#NUM!), (N-acetylglutamate.synthase.deficiency:0:4879:0:6:1.00:#NUM!:0.00:#NUM!), (Neonatal.pneumoni a:0:4879:0:6:1.00:#NUM!:0.00:#NUM!), (Neonatal.respiratory.failure:0:4879:0:6:1.00:#NUM!:0 0.00:#NUM!), (Nicotinic.acid.deficiency:0:4879:0:6:1.00:#NUM!:0.00:#NUM!), (Night.blindness: 0:4879:0:6:1.00:#NUM!:0.00:#NUM!), (Nipple.neoplasm:0:4879:0:6:1.00:#NUM!:0.00:#NUM!), (NUT.midline.carcinoma:0:4879:0:6:1.00:#NUM!:0.00:#NUM!), (Oesophageal.oedema:0:4879: 0:6:1.00:#NUM!:0.00:#NUM!), (Oocyte.donation:0:4879:0:6:1.00:#NUM!:0.00:#NUM!), (Opiate s:0:4879:0:6:1.00:#NUM!:0.00:#NUM!), (Oral.mucosal.exfoliation:0:4879:0:6:1.00:#NUM!:0.00: #NUM!), (Otorhinolaryngological.surgery:0:4879:0:6:1.00:#NUM!:0.00:#NUM!), (Ovarian.repai r:0:4879:0:6:1.00:#NUM!:0.00:#NUM!), (Pallor:0:4879:0:6:1.00:#NUM!:0.00:#NUM!), (Passive. smoking:0:4879:0:6:1.00:#NUM!:0.00:#NUM!), (Periarthritis.calcarea:0:4879:0:6:1.00:#NUM!: 0.00:#NUM!), (Pericoronitis:0:4879:0:6:1.00:#NUM!:0.00:#NUM!), (Perineal.infection:0:4879:0: 6:1.00:#NUM!:0.00:#NUM!), (Pharyngeal.cancer.stage.IV:0:4879:0:6:1.00:#NUM!:0.00:#NUM!), (Pharyngeal.lesion:0:4879:0:6:1.00:#NUM!:0.00:#NUM!), (Pituitary.cancer.metastatic:0:4879:0: 6:1.00:#NUM!:0.00:#NUM!), (Pleural.sarcoma:0:4879:0:6:1.00:#NUM!:0.00:#NUM!), (Polio.im munisation:0:4879:0:6:1.00:#NUM!:0.00:#NUM!), (Post-traumatic.neuralgia:0:4879:0:6:1.00:#NUM!:0.00:#NUM!), (Post.procedural.sepsis:0:4879:0:6:1. 00:#NUM!:0.00:#NUM!), (Postpartum.sepsis:0:4879:0:6:1.00:#NUM!:0.00:#NUM!), (Product.fo rmulation.issue:0:4879:0:6:1.00:#NUM!:0.00:#NUM!), (Propionibacterium.infection:0:4879:0:6: 1.00:#NUM!:0.00:#NUM!), (Puerperal.infection:0:4879:0:6:1.00:#NUM!:0.00:#NUM!), (Pulmon ary.echinococciasis:0:4879:0:6:1.00:#NUM!:0.00:#NUM!), (Pulmonary.vein.occlusion:0:4879:0: 6:1.00:#NUM!:0.00:#NUM!), (Pupillary.disorder:0:4879:0:6:1.00:#NUM!:0.00:#NUM!), (Pyrami dal.tract.syndrome:0:4879:0:6:1.00:#NUM!:0.00:#NUM!), (Radiologically.isolated.syndrome:0:4 879:0:6:1.00:#NUM!:0.00:#NUM!), (Radiotherapy.to.prostate:0:4879:0:6:1.00:#NUM!:0.00:#N UM!), (Radiotherapy.to.soft.tissue:0:4879:0:6:1.00:#NUM!:0.00:#NUM!), (Red.blood.cell.analysi s.abnormal:0:4879:0:6:1.00:#NUM!:0.00:#NUM!), (Renal.salt-wasting.syndrome:0:4879:0:6:1.00:#NUM!:0.00:#NUM!), (Respiratory.tract.carcinoma.in.situ:0: 4879:0:6:1.00:#NUM!:0.00:#NUM!), (Rhabdomyoma:0:4879:0:6:1.00:#NUM!:0.00:#NUM!), (R ubella.immunisation:0:4879:0:6:1.00:#NUM!:0.00:#NUM!), (Scrotal.oedema:0:4879:0:6:1.00:#NUM!:0.00:#NUM!), (Skin.sensitisation:0:4879:0:6:1.00:#NUM!:0.00:#NUM!), (Small.intestinal.perforation:0:4879:0:6:1.00:#NUM!:0.00:#NUM!), (Somatisation.disorder:0:4879:0:6:1.00:#NU M!:0.00:#NUM!), (Splenic.candidiasis:0:4879:0:6:1.00:#NUM!:0.00:#NUM!), (Stenotrophomona s.sepsis:0:4879:0:6:1.00:#NUM!:0.00:#NUM!), (Stenotrophomonas.test.positive:0:4879:0:6:1.00: #NUM!:0.00:#NUM!), (Streptococcal.abscess:0:4879:0:6:1.00:#NUM!:0.00:#NUM!), (Stroke-like.migraine.attacks.after.radiation.therapy:0:4879:0:6:1.00:#NUM!:0.00:#NUM!), (Sympathetic.nerve.destruction:0:4879:0:6:1.00:#NUM!:0.00:#NUM!), (Tendon.discomfort:0:4879:0:6:1.00:#NUM!:0.00:#NUM!), (Thoracic.operation:0:4879:0:6:1.00:#NUM!:0.00:#NUM!), (Thromboelast ogram:0:4879:0:6:1.00:#NUM!:0.00:#NUM!), (Thyroid.stimulating.hormone-producing.pituitary.tumour:0:4879:0:6:1.00:#NUM!:0.00:#NUM!), (Thyroxine.increased:0:4879: 0:6:1.00:#NUM!:0.00:#NUM!), (Total.bile.acids:0:4879:0:6:1.00:#NUM!:0.00:#NUM!), (Transm yocardial.revascularisation:0:4879:0:6:1.00:#NUM!:0.00:#NUM!), (Urinary.sediment.abnormal: 0:4879:0:6:1.00:#NUM!:0.00:#NUM!), (Urogenital.atrophy:0:4879:0:6:1.00:#NUM!:0.00:#NU M!), (Uterine.repair:0:4879:0:6:1.00:#NUM!:0.00:#NUM!), (Vascular.procedure.complication:0: 4879:0:6:1.00:#NUM!:0.00:#NUM!), (Vertebral.artery.hypoplasia:0:4879:0:6:1.00:#NUM!:0.00: #NUM!), (Vitamin.A.decreased:0:4879:0:6:1.00:#NUM!:0.00:#NUM!), (Vitamin.A.deficiency.ey e.disorder:0:4879:0:6:1.00:#NUM!:0.00:#NUM!), (Vocal.cord.polypectomy:0:4879:0:6:1.00:#N UM!:0.00:#NUM!), (Vulvar.dysplasia:0:4879:0:6:1.00:#NUM!:0.00:#NUM!), (Non-Hodgkin's.lymphoma.unspecified.histology.aggressive:11:4868:0:6:1.00:1.00:0.65:0.79), (Hodgk in's.disease.mixed.cellularity.recurrent:8:4871:0:6:1.00:1.00:0.57:0.73), (Autoimmune.inner.ear.d isease:7:4872:0:6:1.00:1.00:0.54:0.70), (Chromoblastomycosis:7:4872:0:6:1.00:1.00:0.54:0.70), (Eye.infection.syphilitic:7:4872:0:6:1.00:1.00:0.54:0.70), (Varicella.immunisation:7:4872:0:6:1.0 0:1.00:0.54:0.70), (Mediastinal.disorder:6:4873:0:6:1.00:1.00:0.50:0.67), (Testicular.mass:6:4873: 0:6:1.00:1.00:0.50:0.67), (Juvenile.spondyloarthritis:5:4874:0:6:1.00:1.00:0.45:0.63), (Opisthoton us:5:4874:0:6:1.00:1.00:0.45:0.63), (Scleroedema:5:4874:0:6:1.00:1.00:0.45:0.63), (Sex.hormone. binding.globulin.decreased:5:4874:0:6:1.00:1.00:0.45:0.63), (Angiolipoma:4:4875:0:6:1.00:1.00: 0.40:0.57), (Epidermolysis:4:4875:0:6:1.00:1.00:0.40:0.57), (Hepatitis.B.surface.antigen.positive: 4:4875:0:6:1.00:1.00:0.40:0.57), (Thyroid.B-cell.lymphoma:4:4875:0:6:1.00:1.00:0.40:0.57), (Vaginal.fistula:4:4875:0:6:1.00:1.00:0.40:0.57), (Antineutrophil.cytoplasmic.antibody.increased:3:4876:0:6:1.00:1.00:0.33:0.50), (Bladder.transiti onal.cell.carcinoma.recurrent:3:4876:0:6:1.00:1.00:0.33:0.50), (Colon.cancer.stage.0:3:4876:0:6: 1.00:1.00:0.33:0.50), (Cronkhite-Canada.syndrome:3:4876:0:6:1.00:1.00:0.33:0.50), (Electroencephalogram:3:4876:0:6:1.00:1.00: 0.33:0.50), (Hodgkin's.disease.nodular.sclerosis.stage.II:3:4876:0:6:1.00:1.00:0.33:0.50), (Paterna 1.drugs.affecting.foetus:3:4876:0:6:1.00:1.00:0.33:0.50), (Solid.pseudopapillary.tumour.of.the.pa ncreas:3:4876:0:6:1.00:1.00:0.33:0.50), (Testicular.embryonal.carcinoma:3:4876:0:6:1.00:1.00:0. 33:0.50), (Tonsillar.neoplasm:3:4876:0:6:1.00:1.00:0.33:0.50), (Tumour.compression:3:4876:0:6: 1.00:1.00:0.33:0.50), (Angiocentric.lymphoma.stage.IV:2:4877:0:6:1.00:1.00:0.25:0.40), (Angiol ymphoid.hyperplasia.with.eosinophilia:2:4877:0:6:1.00:1.00:0.25:0.40), (Blood.gonadotrophin.ab normal:2:4877:0:6:1.00:1.00:0.25:0.40), (Blood.lactate.dehydrogenase:2:4877:0:6:1.00:1.00:0.25: 0.40), (C-reactive.protein.abnormal:2:4877:0:6:1.00:1.00:0.25:0.40), (Chorioretinal.scar:2:4877:0:6:1.00:1. 00:0.25:0.40), (Cryptococcal.cutaneous.infection:2:4877:0:6:1.00:1.00:0.25:0.40), (Cytomegalovi rus.gastrointestinal.infection:2:4877:0:6:1.00:1.00:0.25:0.40), (Elbow.deformity:2:4877:0:6:1.00: 1.00:0.25:0.40), (Faecal.volume.decreased:2:4877:0:6:1.00:1.00:0.25:0.40), (Feeding.tube.compli cation:2:4877:0:6:1.00:1.00:0.25:0.40), (Fungal.cystitis:2:4877:0:6:1.00:1.00:0.25:0.40), (Giant.ce lltumour.of.tendon.sheath:2:4877:0:6:1.00:1.00:0.25:0.40), (Haptoglobin.decreased:2:4877:0:6:1 0.00:1.00:0.25:0.40), (Hepatitis.neonatal:2:4877:0:6:1.00:1.00:0.25:0.40), (Hypotriglyceridaemia:2: 4877:0:6:1.00:1.00:0.25:0.40), (Inflammatory.pseudotumour:2:4877:0:6:1.00:1.00:0.25:0.40), (Isc haemic.skin.ulcer:2:4877:0:6:1.00:1.00:0.25:0.40), (Latent.tetany:2:4877:0:6:1.00:1.00:0.25:0.40), (Osteoprotegerin:2:4877:0:6:1.00:1.00:0.25:0.40), (Otosclerosis:2:4877:0:6:1.00:1.00:0.25:0.40), (Ovarian.epithelial.cancer.stage.III:2:4877:0:6:1.00:1.00:0.25:0.40), (Pelvic.mass:2:4877:0:6:1. 00:1.00:0.25:0.40), (Pneumomediastinum:2:4877:0:6:1.00:1.00:0.25:0.40), (Primary.mediastinal.l arge.B-cell.lymphoma.stage.II:2:4877:0:6:1.00:1.00:0.25:0.40), (Rectosigmoid.cancer.stage.III:2:4877:0: 6:1.00:1.00:0.25:0.40), (Sebaceous.carcinoma:2:4877:0:6:1.00:1.00:0.25:0.40), (Synovial.fluid.cr ystal.present:2:4877:0:6:1.00:1.00:0.25:0.40), (Vasculitic.ulcer:2:4877:0:6:1.00:1.00:0.25:0.40), (Acariasis:1:4878:0:6:1.00:1.00:0.14:0.25), (Acral.lentiginous.melanoma.stage.II:1:4878:0:6:1.00: 1.00:0.14:0.25), (Ainhum:1:4878:0:6:1.00:1.00:0.14:0.25), (Alpha.1.foetoprotein.increased:1:487 8:0:6:1.00:1.00:0.14:0.25), (Anorectal.manometry:1:4878:0:6:1.00:1.00:0.14:0.25), (Anti-ganglioside.antibody.positive:1:4878:0:6:1.00:1.00:0.14:0.25), (Benign.cardiac.neoplasm:1:4878: 0:6:1.00:1.00:0.14:0.25), (Bladder.necrosis:1:4878:0:6:1.00:1.00:0.14:0.25), (Bladder.tamponade: 1:4878:0:6:1.00:1.00:0.14:0.25), (Bronchomalacia:1:4878:0:6:1.00:1.00:0.14:0.25), (Cardiac.asth ma:1:4878:0:6:1.00:1.00:0.14:0.25), (Carotid.artery.insufficiency:1:4878:0:6:1.00:1.00:0.14:0.25), (Cerebrovascular.stenosis:1:4878:0:6:1.00:1.00:0.14:0.25), (CHAD2DS2-VASc. score.increased:1:4878:0:6:1.00:1.00:0.14:0.25), (CHARGE.syndrome:1:4878:0:6:1.00:1.0 0:0.14:0.25), (Chloracne:1:4878:0:6:1.00:1.00:0.14:0.25), (Circulating.anticoagulant.positive:1:48 78:0:6:1.00:1.00:0.14:0.25), (Coagulation.factor.level.normal:1:4878:0:6:1.00:1.00:0.14:0.25), (C ongenital.anaemia:1:4878:0:6:1.00:1.00:0.14:0.25), (Connective.tissue.inflammation:1:4878:0:6: 1.00:1.00:0.14:0.25), (Corneal.infiltrates:1:4878:0:6:1.00:1.00:0.14:0.25), (Cyst.aspiration:1:4878: 0:6:1.00:1.00:0.14:0.25), (Desmoplastic.melanoma:1:4878:0:6:1.00:1.00:0.14:0.25), (Diverticulu m.oesophageal:1:4878:0:6:1.00:1.00:0.14:0.25), (Epinephrine.abnormal:1:4878:0:6:1.00:1.00:0.1 4:0.25), (Flatback.syndrome:1:4878:0:6:1.00:1.00:0.14:0.25), (Fracture.nonunion:1:4878:0:6:1.00: 1.00:0.14:0.25), (Gallbladder.cancer.recurrent:1:4878:0:6:1.00:1.00:0.14:0.25), (Glucose.toleranc e.impaired.in.pregnancy:1:4878:0:6:1.00:1.00:0.14:0.25), (Haemorrhagic.fever.with.renal.syndro me:1:4878:0:6:1.00:1.00:0.14:0.25), (Hepatic.artery.stenosis:1:4878:0:6:1.00:1.00:0.14:0.25), (Hi gh.risk.sexual.behaviour:1:4878:0:6:1.00:1.00:0.14:0.25), (Human.papilloma.virus.test.positive:1: 4878:0:6:1.00:1.00:0.14:0.25), (Human.placental.lactogen.increased:1:4878:0:6:1.00:1.00:0.14:0 0.25), (Intrathecal.pump.insertion:1:4878:0:6:1.00:1.00:0.14:0.25), (Lemierre.syndrome:1:4878:0:6:1.00:1.00:0.14:0.25), (Lumbar.spine.flattening:1:4878:0:6:1.00:1.00:0.14:0.25), (Lung.squamous. cell.carcinoma.stage.II:1:4878:0:6:1.00:1.00:0.14:0.25), (Lupus.vasculitis:1:4878:0:6:1.00:1.00:0. 14:0.25), (Malignant.neoplasm.of.auricular.cartilage:1:4878:0:6:1.00:1.00:0.14:0.25), (Microsatell ite.instability.cancer:1:4878:0:6:1.00:1.00:0.14:0.25), (Neutrophilia:1:4878:0:6:1.00:1.00:0.14:0. 25), (Noninfective.chorioretinitis:1:4878:0:6:1.00:1.00:0.14:0.25), (Notalgia.paraesthetica:1:4878: 0:6:1.00:1.00:0.14:0.25), (Obstructive.uropathy:1:4878:0:6:1.00:1.00:0.14:0.25), (Office.visit:1:4 878:0:6:1.00:1.00:0.14:0.25), (Oscillopsia:1:4878:0:6:1.00:1.00:0.14:0.25), (Ovarian.adenoma:1:4 878:0:6:1.00:1.00:0.14:0.25), (Ovarian.germ.cell.cancer.stage.III:1:4878:0:6:1.00:1.00:0.14:0.25), (Pancreatolithiasis:1:4878:0:6:1.00:1.00:0.14:0.25), (Papilloma.excision:1:4878:0:6:1.00:1.00:0. 14:0.25), (Papulopustular.rosacea:1:4878:0:6:1.00:1.00:0.14:0.25), (Platelet.adhesiveness.abnorm al:1:4878:0:6:1.00:1.00:0.14:0.25), (Pneumonia.lipoid:1:4878:0:6:1.00:1.00:0.14:0.25), (Prostate. cancer.stage.Ill:1:4878:0:6:1.00:1.00:0.14:0.25), (Pulmonary.artery.aneurysm:1:4878:0:6:1.00:1. 00:0.14:0.25), (Pulmonary.microemboli:1:4878:0:6:1.00:1.00:0.14:0.25), (Renal.artery.thrombosis: 1:4878:0:6:1.00:1.00:0.14:0.25), (Retinal.dystrophy:1:4878:0:6:1.00:1.00:0.14:0.25), (Self.estee m.decreased:1:4878:0:6:1.00:1.00:0.14:0.25), (Seminal.vesicular.infection:1:4878:0:6:1.00:1.00: 0.14:0.25), (Squamous.endometrial.carcinoma:1:4878:0:6:1.00:1.00:0.14:0.25), (Stickler's.syndro me:1:4878:0:6:1.00:1.00:0.14:0.25), (Sydenham's.chorea:1:4878:0:6:1.00:1.00:0.14:0.25), (Syste mic-pulmonary.artery. shunt:1:4878:0:6:1.00:1.00:0.14:0.25), (Tongue.paralysis:1:4878:0:6:1.00:1.00: 0.14:0.25), (Toxic.shock.syndrome.staphylococcal:1:4878:0:6:1.00:1.00:0.14:0.25), (Traumatic.ha emothorax:1:4878:0:6:1.00:1.00:0.14:0.25), (Tubulointerstitial.nephritis.and.uveitis.syndrome:1:4 878:0:6:1.00:1.00:0.14:0.25), (Ureteric.cancer.recurrent:1:4878:0:6:1.00:1.00:0.14:0.25), (Urethral. syndrome:1:4878:0:6:1.00:1.00:0.14:0.25), (Uterine.operation:1:4878:0:6:1.00:1.00:0.14:0.25), (Vegetarian:1:4878:0:6:1.00:1.00:0.14:0.25), (Victim.of.sexual.abuse:1:4878:0:6:1.00:1.00:0.14:0. 25), (Waxy.flexibility:1:4878:0:6:1.00:1.00:0.14:0.25), (Xanthomatosis:1:4878:0:6:1.00:1.00:0.14: 0.25), (Abdominal.wall.haemorrhage:0:4878:0:7:1.00:#NUM!:0.00:#NUM!), (Accommodation.d isorder:0:4878:0:7:1.00:#NUM!:0.00:#NUM!), (Acrokeratosis.paraneoplastica:0:4878:0:7:1.00:#NUM!:0.00:#NUM!), (ADAMTS13. activity.decreased:0:4878:0:7:1.00:#NUM!:0.00:#NUM!), (A denotonsillectomy:0:4878:0:7:1.00:#NUM!:0.00:#NUM!), (Alpha-1.anti-trypsin:0:4878:0:7:1.00:#NUM!:0.00:#NUM!), (Anal.sphincter.atony:0:4878:0:7:1.00:#NUM!:0. 00:#NUM!), (Anal.ulcer.haemorrhage:0:4878:0:7:1.00:#NUM!:0.00:#NUM!), (Androgens.increas ed:0:4878:0:7:1.00:#NUM!:0.00:#NUM!), (Anthrax:0:4878:0:7:1.00:#NUM!:0.00:#NUM!), (Aor tic.dilatation:0:4878:0:7:1.00:#NUM!:0.00:#NUM!), (Aortic.valve.atresia:0:4878:0:7:1.00:#NU M!:0.00:#NUM!), (Aortogram:0:4878:0:7:1.00:#NUM!:0.00:#NUM!), (Apparent.death:0:4878:0: 7:1.00:#NUM!:0.00:#NUM!), (Application.site.irritation:0:4878:0:7:1.00:#NUM!:0.00:#NUM!), (Arteriogram.coronary.abnormal:0:4878:0:7:1.00:#NUM!:0.00:#NUM!), (Auditory. disorder:0:487 8:0:7:1.00:#NUM!:0.00:#NUM!), (B-cell.small.lymphocytic.lymphoma.stage.IV:0:4878:0:7:1.00:#NUM!:0.00:#NUM!), (Bartholinitis: 0:4878:0:7:1.00:#NUM!:0.00:#NUM!), (Biopsy.breast:0:4878:0:7:1.00:#NUM!:0.00:#NUM!), ( Bladder.cancer.stage.II:0:4878:0:7:1.00:#NUM!:0.00:#NUM!), (Blepharal.pigmentation:0:4878:0: 7:1.00:#NUM!:0.00:#NUM!), (Blepharoplasty:0:4878:0:7:1.00:#NUM!:0.00:#NUM!), (Blood.ch olinesterase:0:4878:0:7:1.00:#NUM!:0.00:#NUM!), (Bone.abscess:0:4878:0:7:1.00:#NUM!:0.00: #NUM!), (Bone.marrow.reticulin.fibrosis:0:4878:0:7:1.00:#NUM!:0.00:#NUM!), (Borrelia.test.p ositive:0:4878:0:7:1.00:#NUM!:0.00:#NUM!), (Bronchoalveolar.lavage:0:4878:0:7:1.00:#NUM!: 0.00:#NUM!), (Brown-Sequard.syndrome:0:4878:0:7:1.00:#NUM!:0.00:#NUM!), (Capnocytophaga.infection:0:4878:0: 7:1.00:#NUM!:0.00:#NUM!), (Capsular.waming.syndrome:0:4878:0:7:1.00:#NUM!:0.00:#NUM !), (Cardiac.autonomic.neuropathy:0:4878:0:7:1.00:#NUM!:0.00:#NUM!), (Cardiac.function.test. abnormal:0:4878:0:7:1.00:#NUM!:0.00:#NUM!), (Carotid.artery.restenosis:0:4878:0:7:1.00:#NU M!:0.00:#NUM!), (CD20.antigen.positive:0:4878:0:7:1.00:#NUM!:0.00:#NUM!), (Cerebral. candi diasis:0:4878:0:7:1.00:#NUM!:0.00:#NUM!), (Chondromalacia:0:4878:0:7:1.00:#NUM!:0.00:#N UM!), (Colon.cancer.stage.I:0:4878:0:7:1.00:#NUM!:0.00:#NUM!), (Congenital.hiatus.hernia:0:4 878:0:7:1.00:#NUM!:0.00:#NUM!), (Culture:0:4878:0:7:1.00:#NUM!:0.00:#NUM!), (Demented. relative:0:4878:0:7:1.00:#NUM!:0.00:#NUM!), (Dentatorubral-pallidoluysian.atrophy:0:4878:0:7:1.00:#NUM!:0.00:#NUM!), (Disaccharide.metabolism.disorde r:0:4878:0:7:1.00:#NUM!:0.00:#NUM!), (Dislocation.of.sternum:0:4878:0:7:1.00:#NUM!:0.00:#NUM!), (Ear.lobe.infection:0:4878:0:7:1.00:#NUM!:0.00:#NUM!), (EGFR.gene.overexpression: 0:4878:0:7:1.00:#NUM!:0.00:#NUM!), (Endocrine.pancreatic.disorder:0:4878:0:7:1.00:#NUM!: 0.00:#NUM!), (Exposure.via.inhalation:0:4878:0:7:1.00:#NUM!:0.00:#NUM!), (Eyelash.thickeni ng:0:4878:0:7:1.00:#NUM!:0.00:#NUM!), (Facial.operation:0:4878:0:7:1.00:#NUM!:0.00:#NU M!), (False.labour:0:4878:0:7:1.00:#NUM!:0.00:#NUM!), (Feeding.tube.user:0:4878:0:7:1.00:#N UM!:0.00:#NUM!), (Functional.residual.capacity:0:4878:0:7:1.00:#NUM!:0.00:#NUM!), (Gastric.cancer.stage.II:0:4878:0:7:1.00:#NUM!:0.00:#NUM!), (Gilbert's.syndrome:0:4878:0:7:1.00:#N UM!:0.00:#NUM!), (Glare:0:4878:0:7:1.00:#NUM!:0.00:#NUM!), (Glomus.tumour:0:4878:0:7:1. 00:#NUM!:0.00:#NUM!), (Glucose.tolerance.decreased:0:4878:0:7:1.00:#NUM!:0.00:#NUM!), (Glucose.tolerance.test.abnormal:0:4878:0:7:1.00:#NUM!:0.00:#NUM!), (Granulomatous.liver.di sease:0:4878:0:7:1.00:#NUM!:0.00:#NUM!), (Haemophilus.test.positive:0:4878:0:7:1.00:#NUM !:0.00:#NUM!), (Heart.valve.explantation:0:4878:0:7:1.00:#NUM!:0.00:#NUM!), (Heat.stroke:0: 4878:0:7:1.00:#NUM!:0.00:#NUM!), (Hepatitis.C.RNA.increased:0:4878:0:7:1.00:#NUM!:0.00: #NUM!), (Herpes.sepsis:0:4878:0:7:1.00:#NUM!:0.00:#NUM!), (HIV.antibody:0:4878:0:7:1.00:#NUM!:0.00:#NUM!), (Hodgkin's.disease.lymphocyte.depletion.type.stage.IV:0:4878:0:7:1.00:#N UM!:0.00:#NUM!), (Hookworm.infection:0:4878:0:7:1.00:#NUM!:0.00:#NUM!), (Horner's.synd rome:0:4878:0:7:1.00:#NUM!:0.00:#NUM!), (Human.ehrlichiosis:0:4878:0:7:1.00:#NUM!:0.00: #NUM!), (Human.epidermal.growth.factor.receptor.increased:0:4878:0:7:1.00:#NUM!:0.00:#NU M!), (Hypotonia.neonatal:0:4878:0:7:1.00:#NUM!:0.00:#NUM!), (Influenza.B.virus.test.positive: 0:4878:0:7:1.00:#NUM!:0.00:#NUM!), (Intensive.care:0:4878:0:7:1.00:#NUM!:0.00:#NUM!), (I ntestinal.metastasis:0:4878:0:7:1.00:#NUM!:0.00:#NUM!), (Intra-amniotic.injection:0:4878:0:7:1.00:#NUM!:0.00:#NUM!), (Intraocular.haematoma:0:4878:0:7:1. 00:#NUM!:0.00:#NUM!), (Language.disorder:0:4878:0:7:1.00:#NUM!:0.00:#NUM!), (Large.cell lung. cancer.recurrent:0:4878:0:7:1.00:#NUM!:0.00:#NUM!), (Laryngeal.ulceration:0:4878:0:7:1 0.00:#NUM!:0.00:#NUM!), (Light.chain.analysis.abnormal:0:4878:0:7:1.00:#NUM!:0.00:#NUM!), (Limited.symptom.panic.attack:0:4878:0:7:1.00:#NUM!:0.00:#NUM!), (Lipomatosis:0:4878:0: 7:1.00:#NUM!:0.00:#NUM!), (Loss.of.control.of.legs:0:4878:0:7:1.00:#NUM!:0.00:#NUM!), (Lo w.density.lipoprotein.apheresis:0:4878:0:7:1.00:#NUM!:0.00:#NUM!), (Lupus.pancreatitis:0:487 8:0:7:1.00:#NUM!:0.00:#NUM!), (Lymphadenitis.fungal:0:4878:0:7:1.00:#NUM!:0.00:#NUM!), (Malignant.mesenchymoma.metastatic:0:4878:0:7:1.00:#NUM!:0.00:#NUM!), (Malignant.neopl asm. of.cornea:0:4878:0:7:1.00:#NUM!:0.00:#NUM!), (Mastocytic.enterocolitis:0:4878:0:7:1.00: #NUM!:0.00:#NUM!), (Medical.device.site.thrombosis:0:4878:0:7:1.00:#NUM!:0.00:#NUM!), (Nasal.polypectomy:0:4878:0:7:1.00:#NUM!:0.00:#NUM!), (Nonketotic.hyperglycinaemia:0:487 8:0:7:1.00:#NUM!:0.00:#NUM!), (Oesophageal.food.impaction:0:4878:0:7:1.00:#NUM!:0.00:#NUM!), (Optic.nerve.compression:0:4878:0:7:1.00:#NUM!:0.00:#NUM!), (Oral.pruritus:0:4878: 0:7:1.00:#NUM!:0.00:#NUM!), (Otitis.media.bacterial:0:4878:0:7:1.00:#NUM!:0.00:#NUM!), (O varian.remnant. syndrome:0:4878:0:7:1.00:#NUM!:0.00:#NUM!), (Overgrowth. fungal:0:4878:0:7 0.1.00:#NUM!:0.00:#NUM!), (Painful.defaecation:0:4878:0:7:1.00:#NUM!:0.00:#NUM!), (Pancre atic.carcinoma.stage.II:0:4878:0:7:1.00:#NUM!:0.00:#NUM!), (Pancreatic.operation:0:4878:0:7: 1.00:#NUM!:0.00:#NUM!), (Parasite.stool.test.positive:0:4878:0:7:1.00:#NUM!:0.00:#NUM!), (Patient.restraint:0:4878:0:7:1.00:#NUM!:0.00:#NUM!), (Penis.carcinoma.metastatic:0:4878:0:7: 1.00:#NUM!:0.00:#NUM!), (Periarticular.disorder:0:4878:0:7:1.00:#NUM!:0.00:#NUM!), (Perio stitis:0:4878:0:7:1.00:#NUM!:0.00:#NUM!), (Peritonsillitis:0:4878:0:7:1.00:#NUM!:0.00:#NUM !), (Post.procedural.urine.leak:0:4878:0:7:1.00:#NUM!:0.00:#NUM!), (Procalcitonin.increased:0: 4878:0:7:1.00:#NUM!:0.00:#NUM!), (Psychiatric.evaluation:0:4878:0:7:1.00:#NUM!:0.00:#NU M!), (Psychogenic.tremor:0:4878:0:7:1.00:#NUM!:0.00:#NUM!), (Rabies:0:4878:0:7:1.00:#NU M!:0.00:#NUM!), (Radial.nerve.injury:0:4878:0:7:1.00:#NUM!:0.00:#NUM!), (Radiculotomy:0: 4878:0:7:1.00:#NUM!:0.00:#NUM!), (Red.blood.cell.analysis:0:4878:0:7:1.00:#NUM!:0.00:#N UM!), (Reduced.bladder.capacity:0:4878:0:7:1.00:#NUM!:0.00:#NUM!), (Reexpansion.pulmonar y. oedema:0:4878:0:7:1.00:#NUM!:0.00:#NUM!), (Residual.urine.volume.increased:0:4878:0:7:1 0.00:#NUM!:0.00:#NUM!), (Retinal.artery.embolism:0:4878:0:7:1.00:#NUM!:0.00:#NUM!), (Reti noic.acid.syndrome:0:4878:0:7:1.00:#NUM!:0.00:#NUM!), (Retroperitoneal.haematoma:0:4878: 0:7:1.00:#NUM!:0.00:#NUM!), (Salivary.duct.obstruction:0:4878:0:7:1.00:#NUM!:0.00:#NUM!), (Sarcomatosis:0:4878:0:7:1.00:#NUM!:0.00:#NUM!), (School.refusal:0:4878:0:7:1.00:#NUM!: 0.00:#NUM!), (Sepsis.pasteurella:0:4878:0:7:1.00:#NUM!:0.00:#NUM!), (Shigella.infection:0:48 78:0:7:1.00:#NUM!:0.00:#NUM!), (Sinus.antrostomy:0:4878:0:7:1.00:#NUM!:0.00:#NUM!), (Sk in.injury:0:4878:0:7:1.00:#NUM!:0.00:#NUM!), (Small.intestine.leiomyosarcoma:0:4878:0:7:1.0 0:#NUM!:0.00:#NUM!), (Spermatocele:0:4878:0:7:1.00:#NUM!:0.00:#NUM!), (Subcomeal.pust ular.dermatosis:0:4878:0:7:1.00:#NUM!:0.00:#NUM!), (Trabeculoplasty:0:4878:0:7:1.00:#NUM !:0.00:#NUM!), (Traumatic.arthropathy:0:4878:0:7:1.00:#NUM!:0.00:#NUM!), (Traumatic.renal. injury:0:4878:0:7:1.00:#NUM!:0.00:#NUM!), (Tryptase.increased:0:4878:0:7:1.00:#NUM!:0.00: #NUM!), (Tumour.cell.mobilisation:0:4878:0:7:1.00:#NUM!:0.00:#NUM!), (Upper.airway.resist ance.syndrome:0:4878:0:7:1.00:#NUM!:0.00:#NUM!), (Urinary.tract.infection.staphylococcal:0: 4878:0:7:1.00:#NUM!:0.00:#NUM!), (Urine.potassium.abnormal:0:4878:0:7:1.00:#NUM!:0.00:#NUM!), (Uterine.hyperstimulation:0:4878:0:7:1.00:#NUM!:0.00:#NUM!), (Vaginal.atresia:0:487 8:0:7:1.00:#NUM!:0.00:#NUM!), (Vaginal.lesion:0:4878:0:7:1.00:#NUM!:0.00:#NUM!), (Vascul ar.access.site.inflammation:0:4878:0:7:1.00:#NUM!:0.00:#NUM!), (Vascular.graft.stenosis:0:48 78:0:7:1.00:#NUM!:0.00:#NUM!), (Venous.pressure.abnormal:0:4878:0:7:1.00:#NUM!:0.00:#N UM!), (Viral.myelitis:0:4878:0:7:1.00:#NUM!:0.00:#NUM!), (Vocal.cord.dysfunction:0:4878:0:7: 1.00:#NUM!:0.00:#NUM!), (Vulval.abscess:0:4878:0:7:1.00:#NUM!:0.00:#NUM!), (Writer's.cra mp:0:4878:0:7:1.00:#NUM!:0.00:#NUM!), (Anaplastic.large.cell.lymphoma.T-.and.null-cell.types.stage.IV:12:4866:0:7:1.00:1.00:0.63:0.77), (Immunoblastic.lymphoma:12:4866:0:7:1.0 0:1.00:0.63:0.77), (Non-Hodgkin's.lymphoma.stage.I:10:4868:0:7:1.00:1.00:0.59:0.74), (B-cell.unclassifiable.lymphoma.high.grade:9:4869:0:7:1.00:1.00:0.56:0.72), (Renal.and.pancreas.tra nsplant.rejection:8:4870:0:7:1.00:1.00:0.53:0.70), (Acquired.Von.Willebrand's.disease:6:4872:0: 7:1.00:1.00:0.46:0.63), (Ovarian.germ.cell.teratoma:6:4872:0:7:1.00:1.00:0.46:0.63), (Ovarian.Ser toli-Leydig.cell.tumour:6:4872:0:7:1.00:1.00:0.46:0.63), (Tuberculosis.liver:6:4872:0:7:1.00:1.00:0.4 6:0.63), (Congenital.teratoma:5:4873:0:7:1.00:1.00:0.42:0.59), (Cutaneous.tuberculosis:5:4873:0: 7:1.00:1.00:0.42:0.59), (Labour.stimulation:5:4873:0:7:1.00:1.00:0.42:0.59), (Leukodystrophy:5:4 873:0:7:1.00:1.00:0.42:0.59), (Mantle.cell.lymphoma.stage.III:5:4873:0:7:1.00:1.00:0.42:0.59), (Nasopharyngeal.cancer.stage.IV:5:4873:0:7:1.00:1.00:0.42:0.59), (Nongerminomatous.germ.cell. tumour.of.the.CNS:5:4873:0:7:1.00:1.00:0.42:0.59), (Pyogenic.sterile.arthritis.pyoderma.gangren osum.and.acne.syndrome:5:4873:0:7:1.00:1.00:0.42:0.59), (Serositis:5:4873:0:7:1.00:1.00:0.42:0. 59), (Adrenal.gland.tuberculosis:4:4874:0:7:1.00:1.00:0.36:0.53), (Anal.stenosis:4:4874:0:7:1.00: 1.00:0.36:0.53), (Cell.death:4:4874:0:7:1.00:1.00:0.36:0.53), (Congenital.tuberculosis:4:4874:0:7: 1.00:1.00:0.36:0.53), (Encapsulating.peritoneal.sclerosis:4:4874:0:7:1.00:1.00:0.36:0.53), (Left.ve ntricle.outflow.tract.obstruction:4:4874:0:7:1.00:1.00:0.36:0.53), (Leukaemic.infiltration:4:4874: 0:7:1.00:1.00:0.36:0.53), (Pityriasis.lichenoides.et.varioliformis.acuta:4:4874:0:7:1.00:1.00:0.36: 0.53), (Charles.Bonnet.syndrome:3:4875:0:7:1.00:1.00:0.30:0.46), (Hepatoblastoma.recurrent:3:4 875:0:7:1.00:1.00:0.30:0.46), (Primary.mediastinal.large.B-cell.lymphoma.recurrent:3:4875:0:7:1.00:1.00:0.30:0.46), (Pulmonary.radiation.injury:3:4875:0:7: 1.00:1.00:0.30:0.46), (Splenic.thrombosis:3:4875:0:7:1.00:1.00:0.30:0.46), (Toxic.goitre:3:4875: 0:7:1.00:1.00:0.30:0.46), (Urine.calcium:3:4875:0:7:1.00:1.00:0.30:0.46), (Vascular.neoplasm:3:4 875:0:7:1.00:1.00:0.30:0.46), (Blood.alcohol.increased:2:4876:0:7:1.00:1.00:0.22:0.36), (Bone.sw elling:2:4876:0:7:1.00:1.00:0.22:0.36), (Cervix.carcinoma.stage.III:2:4876:0:7:1.00:1.00:0.22:0.3 6), (Cushingoid:2:4876:0:7:1.00:1.00:0.22:0.36), (Dermatologic.examination:2:4876:0:7:1.00:1.0 0:0.22:0.36), (Diffuse.cutaneous.mastocytosis:2:4876:0:7:1.00:1.00:0.22:0.36), (Ear,.nose.and.thr oat.examination:2:4876:0:7:1.00:1.00:0.22:0.36), (Ear.injury:2:4876:0:7:1.00:1.00:0.22:0.36), (Fa nconi.syndrome.acquired:2:4876:0:7:1.00:1.00:0.22:0.36), (Fibroadenoma.of.breast:2:4876:0:7:1. 00:1.00:0.22:0.36), (Haptoglobin.abnormal:2:4876:0:7:1.00:1.00:0.22:0.36), (Injection.related.rea ction:2:4876:0:7:1.00:1.00:0.22:0.36), (Joint.destruction:2:4876:0:7:1.00:1.00:0.22:0.36), (Laryng eal.cancer.metastatic:2:4876:0:7:1.00:1.00:0.22:0.36), (Malignant.neoplasm.of.eyelid:2:4876:0:7: 1.00:1.00:0.22:0.36), (Mesenteric.fibrosis:2:4876:0:7:1.00:1.00:0.22:0.36), (Mitochondrial.enceph alomyopathy:2:4876:0:7:1.00:1.00:0.22:0.36), (Necrosis.of.artery:2:4876:0:7:1.00:1.00:0.22:0.36), (Oesophageal.adenocarcinoma.stage.III:2:4876:0:7:1.00:1.00:0.22:0.36), (Pancreatoblastoma:2: 4876:0:7:1.00:1.00:0.22:0.36), (Parvovirus.B19.test.positive:2:4876:0:7:1.00:1.00:0.22:0.36), (Pla smodium.malariae.infection:2:4876:0:7:1.00:1.00:0.22:0.36), (Proctocolectomy:2:4876:0:7:1.00: 1.00:0.22:0.36), (Retained.products.of.conception:2:4876:0:7:1.00:1.00:0.22:0.36), (Skeletal.dyspl asia:2:4876:0:7:1.00:1.00:0.22:0.36), (Skin.graft.rejection:2:4876:0:7:1.00:1.00:0.22:0.36), (Tuber culosis.immunisation:2:4876:0:7:1.00:1.00:0.22:0.36), (Vibrio.test.positive:2:4876:0:7:1.00:1.00: 0.22:0.36), (Weber-Christian.disease:2:4876:0:7:1.00:1.00:0.22:0.36), (Adhesiolysis:1:4877:0:7:1.00:1.00:0.13:0.22), (Ankle.arthroplasty:1:4877:0:7:1.00:1.00:0.13:0.22), (Annuloplasty:1:4877:0:7:1.00:1.00:0.13:0. 22), (Anti-epithelial.antibody:1:4877:0:7:1.00:1.00:0.13:0.22), (Aortic.anastomosis:1:4877:0:7:1.00:1.00:0. 13:0.22), (Baltic.myoclonic.epilepsy:1:4877:0:7:1.00:1.00:0.13:0.22), (Benign.neoplasm.of.choroi d:1:4877:0:7:1.00:1.00:0.13:0.22), (Blood.gonadotrophin.increased:1:4877:0:7:1.00:1.00:0.13:0.2 2), (Carbon.dioxide.abnormal:1:4877:0:7:1.00:1.00:0.13:0.22), (Congenital.hyperthyroidism:1:48 77:0:7:1.00:1.00:0.13:0.22), (Congenital.myopathy:1:4877:0:7:1.00:1.00:0.13:0.22), (Conjunctivit is.chlamydial:1:4877:0:7:1.00:1.00:0.13:0.22), (Coombs.direct.test.positive:1:4877:0:7:1.00:1.00: 0.13:0.22), (Degenerative.mitral.valve.disease:1:4877:0:7:1.00:1.00:0.13:0.22), (Diphtheria.immu nisation:1:4877:0:7:1.00:1.00:0.13:0.22), (Encephalitis.eastern.equine:1:4877:0:7:1.00:1.00:0.13: 0.22), (Eye.naevus:1:4877:0:7:1.00:1.00:0.13:0.22), (Fear.of.crowded.places:1:4877:0:7:1.00:1.00: 0.13:0.22), (Foetal.malnutrition:1:4877:0:7:1.00:1.00:0.13:0.22), (Fracture.reduction:1:4877:0:7: 1.00:1.00:0.13:0.22), (Gingival.erosion:1:4877:0:7:1.00:1.00:0.13:0.22), (Graft.loss:1:4877:0:7:1. 00:1.00:0.13:0.22), (Heart.valve.stenosis:1:4877:0:7:1.00:1.00:0.13:0.22), (Hepatitis.virus-associated.nephropathy:1:4877:0:7:1.00:1.00:0.13:0.22), (Holmes.tremor:1:4877:0:7:1.00:1.00:0. 13:0.22), (Immune.reconstitution.inflammatory.syndrome.associated.Kaposi's.sarcoma:1:4877:0: 7:1.00:1.00:0.13:0.22), (Incomplete.precocious.puberty:1:4877:0:7:1.00:1.00:0.13:0.22), (Infected.varicose.vein:1:4877:0:7:1.00:1.00:0.13:0.22), (Injection.site.inflammation:1:4877:0:7:1.00:1.00: 0.13:0.22), (Leptomeningeal.myelomatosis:1:4877:0:7:1.00:1.00:0.13:0.22), (Meniscus.operation: 1:4877:0:7:1.00:1.00:0.13:0.22), (Metabolic.cardiomyopathy:1:4877:0:7:1.00:1.00:0.13:0.22), (Ne onatal.asphyxia:1:4877:0:7:1.00:1.00:0.13:0.22), (Ophthalmic.fluid.drainage:1:4877:0:7:1.00:1.0 0:0.13:0.22), (Oroticaciduria.congenital:1:4877:0:7:1.00:1.00:0.13:0.22), (Papilloma:1:4877:0:7:1 0.00:1.00:0.13:0.22), (Paraganglion.neoplasm.benign:1:4877:0:7:1.00:1.00:0.13:0.22), (Peripheral. nerve.infection:1:4877:0:7:1.00:1.00:0.13:0.22), (Pleural.fibrosis:1:4877:0:7:1.00:1.00:0.13:0.22), (Poor.personal.hygiene:1:4877:0:7:1.00:1.00:0.13:0.22), (Progesterone.abnormal:1:4877:0:7:1.0 0:1.00:0.13:0.22), (Protein.total:1:4877:0:7:1.00:1.00:0.13:0.22), (Salpingectomy:1:4877:0:7:1.00: 1.00:0.13:0.22), (Shortened.cervix:1:4877:0:7:1.00:1.00:0.13:0.22), (Splenic.rupture:1:4877:0:7:1. 00:1.00:0.13:0.22), (Testicular.oedema:1:4877:0:7:1.00:1.00:0.13:0.22), (Tracheal.inflammation:1:4877:0:7:1.00:1.00:0.13:0.22), (Trigeminal.palsy:1:4877:0:7:1.00:1.00:0.13:0.22), (Vaginal.aden ocarcinoma:1:4877:0:7:1.00:1.00:0.13:0.22), (Vena.cava.injury:1:4877:0:7:1.00:1.00:0.13:0.22), (Vesicoureteric.reflux:1:4877:0:7:1.00:1.00:0.13:0.22), (Abdominal.wall.infection:0:4877:0:8:1.0 0:#NUM!:0.00:#NUM!), (Albumin.globulin.ratio.abnormal:0:4877:0:8:1.00:#NUM!:0.00:#NUM !), (Anaesthetic.complication.vascular:0:4877:0:8:1.00:#NUM!:0.00:#NUM!), (Anorectal.operatio n:0:4877:0:8:1.00:#NUM!:0.00:#NUM!), (Antibiotic.level.therapeutic:0:4877:0:8:1.00:#NUM!:0 0.00:#NUM!), (Antiviral.drug.level:0:4877:0:8:1.00:#NUM!:0.00:#NUM!), (Arginase.deficiency:0: 4877:0:8:1.00:#NUM!:0.00:#NUM!), (Barium.swallow:0:4877:0:8:1.00:#NUM!:0.00:#NUM!), (Bartonellosis:0:4877:0:8:1.00:#NUM!:0.00:#NUM!), (Bladder.repair:0:4877:0:8:1.00:#NUM!:0. 00:#NUM!), (Blood.creatine.phosphokinase.BB.increased:0:4877:0:8:1.00:#NUM!:0.00:#NUM!), (Blood.cyanide.increased:0:4877:0:8:1.00:#NUM!:0.00:#NUM!), (Blood.immunoglobulin.A:0:4 877:0:8:1.00:#NUM!:0.00:#NUM!), (Blood.testosterone.free.increased:0:4877:0:8:1.00:#NUM!: 0.00:#NUM!), (Bone.trimming:0:4877:0:8:1.00:#NUM!:0.00:#NUM!), (Brain.herniation:0:4877: 0:8:1.00:#NUM!:0.00:#NUM!), (Breast.prosthesis.implantation:0:4877:0:8:1.00:#NUM!:0.00:#N UM!), (Burkholderia.infection:0:4877:0:8:1.00:#NUM!:0.00:#NUM!), (Caffeine.consumption:0:4 877:0:8:1.00:#NUM!:0.00:#NUM!), (Cardiac.valve.vegetation:0:4877:0:8:1.00:#NUM!:0.00:#N UM!), (Cardiovascular.function.test:0:4877:0:8:1.00:#NUM!:0.00:#NUM!), (Cellulitis.of.male.ex ternal.genital.organ:0:4877:0:8:1.00:#NUM!:0.00:#NUM!), (Cerebellar.haematoma:0:4877:0:8:1. 00:#NUM!:0.00:#NUM!), (Cerebral.amyloid.angiopathy:0:4877:0:8:1.00:#NUM!:0.00:#NUM!), (Chest.wall.mass:0:4877:0:8:1.00:#NUM!:0.00:#NUM!), (Clostridium.bacteraemia:0:4877:0:8:1.0 0:#NUM!:0.00:#NUM!), (Coeliac.artery.stenosis:0:4877:0:8:1.00:#NUM!:0.00:#NUM!), (Congen ital.thrombocyte.disorder:0:4877:0:8:1.00:#NUM!:0.00:#NUM!), (Congo-Crimean.haemorrhagic.fever:0:4877:0:8:1.00:#NUM!:0.00:#NUM!), (Cyst.removal:0:4877:0:8:1. 00:#NUM!:0.00:#NUM!), (Dermatitis.infected:0:4877:0:8:1.00:#NUM!:0.00:#NUM!), (Diabetic. cardiomyopathy:0:4877:0:8:1.00:#NUM!:0.00:#NUM!), (Educational.problem:0:4877:0:8:1.00:#NUM!:0.00:#NUM!), (Electromagnetic.interference:0:4877:0:8:1.00:#NUM!:0.00:#NUM!), (Em bolia.cutis.medicamentosa:0:4877:0:8:1.00:#NUM!:0.00:#NUM!), (Excessive.masturbation:0:48 77:0:8:1.00:#NUM!:0.00:#NUM!), (Eye.ulcer:0:4877:0:8:1.00:#NUM!:0.00:#NUM!), (Faecal.ela stase.concentration.abnormal:0:4877:0:8:1.00:#NUM!:0.00:#NUM!), (Feeling.drunk:0:4877:0:8: 1.00:#NUM!:0.00:#NUM!), (Fibrous.histiocytoma:0:4877:0:8:1.00:#NUM!:0.00:#NUM!), (Flight.of.ideas:0:4877:0:8:1.00:#NUM!:0.00:#NUM!), (Fungal.rhinitis:0:4877:0:8:1.00:#NUM!:0.00:#NUM!), (Gallbladder.necrosis:0:4877:0:8:1.00:#NUM!:0.00:#NUM!), (Gallbladder.polyp:0:4877: 0:8:1.00:#NUM!:0.00:#NUM!), (Gastric.dysplasia:0:4877:0:8:1.00:#NUM!:0.00:#NUM!), (Gastr oenteritis.cryptosporidial:0:4877:0:8:1.00:#NUM!:0.00:#NUM!), (Gaze.palsy:0:4877:0:8:1.00:#NUM!:0.00:#NUM!), (Genetic.counselling:0:4877:0:8:1.00:#NUM!:0.00:#NUM!), (Glycogen.sto rage.disease.type.IV:0:4877:0:8:1.00:#NUM!:0.00:#NUM!), (Haemorrhagic.fever:0:4877:0:8:1.0 0:#NUM!:0.00:#NUM!), (Hepatic.cancer.stage.III:0:4877:0:8:1.00:#NUM!:0.00:#NUM!), (Hepat ic.haemorrhage:0:4877:0:8:1.00:#NUM!:0.00:#NUM!), (HIV-associated.neurocognitive.disorder:0:4877:0:8:1.00:#NUM!:0.00:#NUM!), (HIV.infection.CDC. category.C3:0:4877:0:8:1.00:#NUM!:0.00:#NUM!), (Hypergonadism:0:4877:0:8:1.00:#NUM!:0.0 0:#NUM!), (Hypochondriasis:0:4877:0:8:1.00:#NUM!:0.00:#NUM!), (Hypoplastic.right.heart.syndrome: 0:4877:0:8:1.00:#NUM!:0.00:#NUM!), (Inappropriate.thyroid.stimulating.hormone.secreti on:0:4877:0:8:1.00:#NUM!:0.00:#NUM!), (Incomplete.spinal.fusion:0:4877:0:8:1.00:#NUM!:0.0 0:#NUM!), (Introital.stretch:0:4877:0:8:1.00:#NUM!:0.00:#NUM!), (Iridocomeal.endothelial.syn drome:0:4877:0:8:1.00:#NUM!:0.00:#NUM!), (Iron.binding.capacity.total.decreased:0:4877:0:8: 1.00:#NUM!:0.00:#NUM!), (Joint.stabilisation:0:4877:0:8:1.00:#NUM!:0.00:#NUM!), (Keratitis. viral:0:4877:0:8:1.00:#NUM!:0.00:#NUM!), (Laryngeal.nerve.dysfunction:0:4877:0:8:1.00:#NU M!:0.00:#NUM!), (Lens.disorder:0:4877:0:8:1.00:#NUM!:0.00:#NUM!), (Leuconostoc.infection: 0:4877:0:8:1.00:#NUM!:0.00:#NUM!), (Leukotriene.increased:0:4877:0:8:1.00:#NUM!:0.00:#N UM!), (Mammogram:0:4877:0:8:1.00:#NUM!:0.00:#NUM!), (Median.nerve.injury:0:4877:0:8:1. 00:#NUM!:0.00:#NUM!), (Metastases.to.oesophagus:0:4877:0:8:1.00:#NUM!:0.00:#NUM!), (Mi tochondrial.toxicity:0:4877:0:8:1.00:#NUM!:0.00:#NUM!), (Mycosis.fungoides.stage.II:0:4877:0: 8:1.00:#NUM!:0.00:#NUM!), (Myocardial.depression:0:4877:0:8:1.00:#NUM!:0.00:#NUM!), (Myocardial.necrosis:0:4877:0:8:1.00:#NUM!:0.00:#NUM!), (Nail-patella.syndrome:0:4877:0:8:1.00:#NUM!:0.00:#NUM!), (Nail.bed.disorder:0:4877:0:8:1.00:#N UM!:0.00:#NUM!), (Nasopharyngeal.cancer.stage.III:0:4877:0:8:1.00:#NUM!:0.00:#NUM!), (Ne phritis.autoimmune:0:4877:0:8:1.00:#NUM!:0.00:#NUM!), (Nuclear.magnetic.resonance.imagin g.brain.abnormal:0:4877:0:8:1.00:#NUM!:0.00:#NUM!), (Obsessive.rumination:0:4877:0:8:1.00: #NUM!:0.00:#NUM!), (Onchocerciasis:0:4877:0:8:1.00:#NUM!:0.00:#NUM!), (Oral.mucosal.er ythema:0:4877:0:8:1.00:#NUM!:0.00:#NUM!), (Osmotic.demyelination.syndrome:0:4877:0:8:1. 00:#NUM!:0.00:#NUM!), (Pancreatography:0:4877:0:8:1.00:#NUM!:0.00:#NUM!), (Penile.size.r educed:0:4877:0:8:1.00:#NUM!:0.00:#NUM!), (Peripheral.nerve.injection:0:4877:0:8:1.00:#NU M!:0.00:#NUM!), (Peroneal.muscular.atrophy:0:4877:0:8:1.00:#NUM!:0.00:#NUM!), (Persistent. genital.arousal.disorder:0:4877:0:8:1.00:#NUM!:0.00:#NUM!), (Pharyngeal.mass:0:4877:0:8:1.0 0:#NUM!:0.00:#NUM!), (Platelet-derived.growth.factor.receptor.overexpression:0:4877:0:8:1.00:#NUM!:0.00:#NUM!), (Polyglan dular.autoimmune.syndrome.type.:0:4877:0:8:1.00:#NUM!:0.00:#NUM!), (Polyglandular.autoi mmune.syndrome.type.II:0:4877:0:8:1.00:#NUM!:0.00:#NUM!), (Poriomania:0:4877:0:8:1.00:#NUM!:0.00:#NUM!), (Portal.venous.system.anomaly:0:4877:0:8:1.00:#NUM!:0.00:#NUM!), (Post-traumatic.amnestic.disorder:0:4877:0:8:1.00:#NUM!:0.00:#NUM!), (Postictal.headache:0:4877:0: 8:1.00:#NUM!:0.00:#NUM!), (Preoperative.hormone.treatment:0:4877:0:8:1.00:#NUM!:0.00:#NUM!), (Promiscuity:0:4877:0:8:1.00:#NUM!:0.00:#NUM!), (Prosthetic.vessel.removal:0:4877:0: 8:1.00:#NUM!:0.00:#NUM!), (Prothrombin.time.ratio.decreased:0:4877:0:8:1.00:#NUM!:0.00:#NUM!), (Provisional.tic.disorder:0:4877:0:8:1.00:#NUM!:0.00:#NUM!), (Renal.function.test:0:4 877:0:8:1.00:#NUM!:0.00:#NUM!), (Salpingo-oophorectomy.bilateral:0:4877:0:8:1.00:#NUM!:0.00:#NUM!), (Sedation.complication:0:4877:0: 8:1.00:#NUM!:0.00:#NUM!), (Sickle.cell.trait:0:4877:0:8:1.00:#NUM!:0.00:#NUM!), (Skin.graft.infection:0:4877:0:8:1.00:#NUM!:0.00:#NUM!), (Smoke.sensitivity:0:4877:0:8:1.00:#NUM!:0. 00:#NUM!), (Sopor:0:4877:0:8:1.00:#NUM!:0.00:#NUM!), (Thyroxine.free.decreased:0:4877:0: 8:1.00:#NUM!:0.00:#NUM!), (Tonsillar.disorder:0:4877:0:8:1.00:#NUM!:0.00:#NUM!), (Trache al.dilatation:0:4877:0:8:1.00:#NUM!:0.00:#NUM!), (Tracheal.obstruction:0:4877:0:8:1.00:#NU M!:0.00:#NUM!), (Ulcerated.haemangioma:0:4877:0:8:1.00:#NUM!:0.00:#NUM!), (Unhealthy.d iet:0:4877:0:8:1.00:#NUM!:0.00:#NUM!), (Urethral.adenoma:0:4877:0:8:1.00:#NUM!:0.00:#N UM!), (Vaginal.ulceration:0:4877:0:8:1.00:#NUM!:0.00:#NUM!), (Vertebral.osteophyte:0:4877:0: 8:1.00:#NUM!:0.00:#NUM!), (Vertigo.labyrinthine:0:4877:0:8:1.00:#NUM!:0.00:#NUM!), (Vir al.cardiomyopathy:0:4877:0:8:1.00:#NUM!:0.00:#NUM!), (Perinatal.HIV.infection:25:4852:1:7: 1.00:0.96:0.78:0.86), (HIV.infection.WHO.clinical.stage.IV:11:4866:0:8:1.00:1.00:0.58:0.73), (Mixed.adenoneuroendocrine.carcinoma:11:4866:0:8:1.00:1.00:0.58:0.73), (Rasmussen.encephalitis: 11:4866:0:8:1.00:1.00:0.58:0.73), (Burkitt's.lymphoma.recurrent:9:4868:0:8:1.00:1.00:0.53:0.69), (Ganglioneuroma:9:4868:0:8:1.00:1.00:0.53:0.69), (Intestinal.tuberculosis:9:4868:0:8:1.00:1.00:0 0.53:0.69), (Small.cell.lung.cancer.recurrent:8:4869:0:8:1.00:1.00:0.50:0.67), (Sympathetic.ophthal mia:8:4869:0:8:1.00:1.00:0.50:0.67), (Joint.tuberculosis:7:4870:0:8:1.00:1.00:0.47:0.64), (Acute.h aemorrhagic.ulcerative.colitis:6:4871:0:8:1.00:1.00:0.43:0.60), (B-cell.depletion.therapy:6:4871:0:8:1.00:1.00:0.43:0.60), (Infertility.male:5:4872:0:8:1.00:1.00:0.3 8:0.56), (Leukapheresis:5:4872:0:8:1.00:1.00:0.38:0.56), (Meningococcal.immunisation:5:4872:0: 8:1.00:1.00:0.38:0.56), (Peripheral.T-cell.lymphoma.unspecified.stage.II:5:4872:0:8:1.00:1.00:0.38:0.56), (Telangiectasia:5:4872:0:8:1 0.00:1.00:0.38:0.56), (Cervix.carcinoma.stage.I:4:4873:0:8:1.00:1.00:0.33:0.50), (Complement.fact or.C3.decreased:4:4873:0:8:1.00:1.00:0.33:0.50), (Dacryoadenitis.acquired:4:4873:0:8:1.00:1.00: 0.33:0.50), (Diabetic.endorgan.damage:4:4873:0:8:1.00:1.00:0.33:0.50), (Follicular.dendritic.cell. sarcoma:4:4873:0:8:1.00:1.00:0.33:0.50), (Injection.site.warmth:4:4873:0:8:1.00:1.00:0.33:0.50), (Resorption.bone.increased:4:4873:0:8:1.00:1.00:0.33:0.50), (Testicular.germ.cell.tumour.mixed. stage.III:4:4873:0:8:1.00:1.00:0.33:0.50), (Testis.cancer.recurrent:4:4873:0:8:1.00:1.00:0.33:0.50), (Thymoma.malignant.recurrent:4:4873:0:8:1.00:1.00:0.33:0.50), (Vitello-intestinal.duct.remnant:4:4873:0:8:1.00:1.00:0.33:0.50), (Wound.abscess:4:4873:0:8:1.00:1.00:0. 33:0.50), (Artificial.insemination.by.partner:3:4874:0:8:1.00:1.00:0.27:0.43), (CNS.germinoma:3: 4874:0:8:1.00:1.00:0.27:0.43), (Cytapheresis:3:4874:0:8:1.00:1.00:0.27:0.43), (Cytomegalovirus. hepatitis:3:4874:0:8:1.00:1.00:0.27:0.43), (Decreased.ventricular.afterload:3:4874:0:8:1.00:1.00: 0.27:0.43), (Hepatitis.B.e.antigen:3:4874:0:8:1.00:1.00:0.27:0.43), (Li-Fraumeni.syndrome:3:4874:0:8:1.00:1.00:0.27:0.43), (MacLeod's.syndrome:3:4874:0:8:1.00:1.00:0.27:0.43), (Necrobiosis:3:4874:0:8:1.00:1.00:0.27:0.43), (Nodal.marginal.zone.B-cell.lymphoma.recurrent:3:4874:0:8:1.00:1.00:0.27:0.43), (Penile.vascular.disorder:3:4874:0:8:1. 00:1.00:0.27:0.43), (Postponement.ofpreterm.delivery:3:4874:0:8:1.00:1.00:0.27:0.43), (Proctitis. haemorrhagic:3:4874:0:8:1.00:1.00:0.27:0.43), (Renal.artery.occlusion:3:4874:0:8:1.00:1.00:0.27: 0.43), (Skin.malformation:3:4874:0:8:1.00:1.00:0.27:0.43), (Thyroid.stimulating.hormone.deficiency: 3:4874:0:8:1.00:1.00:0.27:0.43), (Tuberculosis.bladder:3:4874:0:8:1.00:1.00:0.27:0.43), (Wei ght.gain.poor:3:4874:0:8:1.00:1.00:0.27:0.43), (Chikungunya.virus.infection:2:4875:0:8:1.00:1.0 0:0.20:0.33), (Coagulation.factor.V.level:2:4875:0:8:1.00:1.00:0.20:0.33), (Delayed.haemolytic.tr ansfusion.reaction:2:4875:0:8:1.00:1.00:0.20:0.33), (Dihydrotestosterone.increased:2:4875:0:8:1. 00:1.00:0.20:0.33), (Disseminated.varicella.zoster.vaccine.virus.infection:2:4875:0:8:1.00:1.00:0. 20:0.33), (Enterovirus.infection:2:4875:0:8:1.00:1.00:0.20:0.33), (Extraskeletal.ossification:2:487 5:0:8:1.00:1.00:0.20:0.33), (Fat.intolerance:2:4875:0:8:1.00:1.00:0.20:0.33), (Gastroenteritis.aden ovirus:2:4875:0:8:1.00:1.00:0.20:0.33), (Group.B.streptococcus.neonatal.sepsis:2:4875:0:8:1.00: 1.00:0.20:0.33), (Haemangioblastoma:2:4875:0:8:1.00:1.00:0.20:0.33), (Heavy.exposure.to.ultravi olet.light:2:4875:0:8:1.00:1.00:0.20:0.33), (Herpes.simplex.oesophagitis:2:4875:0:8:1.00:1.00:0.2 0:0.33), (Hydrotubation:2:4875:0:8:1.00:1.00:0.20:0.33), (Hypopharyngeal.neoplasm:2:4875:0:8: 1.00:1.00:0.20:0.33), (Inflammatory.carcinoma.of.breast.stage.IV:2:4875:0:8:1.00:1.00:0.20:0.33), (Inhibiting.antibodies:2:4875:0:8:1.00:1.00:0.20:0.33), (Malignant.melanoma.stage.II:2:4875:0: 8:1.00:1.00:0.20:0.33), (Marginal.zone.lymphoma.stage.IV:2:4875:0:8:1.00:1.00:0.20:0.33), (Mo untain.sickness.acute:2:4875:0:8:1.00:1.00:0.20:0.33), (Natural.killer.T.cell.count.increased:2:48 75:0:8:1.00:1.00:0.20:0.33), (Peritonitis.lupus:2:4875:0:8:1.00:1.00:0.20:0.33), (Plasminogen.acti vator.inhibitor.polymorphism:2:4875:0:8:1.00:1.00:0.20:0.33), (Precursor.T-lymphoblastic.lymphoma/leukaemia.stage.III:2:4875:0:8:1.00:1.00:0.20:0.33), (Retinal.scar:2:48 75:0:8:1.00:1.00:0.20:0.33), (Rickets.familial.hypophosphataemic:2:4875:0:8:1.00:1.00:0.20:0.33), (Tricuspid.valve.stenosis:2:4875:0:8:1.00:1.00:0.20:0.33), (Abdominal.wall.neoplasm.malignan t:1:4876:0:8:1.00:1.00:0.11:0.20), (ALK.gene.rearrangement.positive:1:4876:0:8:1.00:1.00:0.11: 0.20), (Aortitis.syphilitic:1:4876:0:8:1.00:1.00:0.11:0.20), (Axillary.lymphadenectomy:1:4876:0:8: 1.00:1.00:0.11:0.20), (Bile.duct.exploration:1:4876:0:8:1.00:1.00:0.11:0.20), (Bile.duct.pressure: 1:4876:0:8:1.00:1.00:0.11:0.20), (Bladder.hypertrophy:1:4876:0:8:1.00:1.00:0.11:0.20), (Blood.fo llicle.stimulating.hormone:1:4876:0:8:1.00:1.00:0.11:0.20), (Blood.immunoglobulin.M.decreased: 1:4876:0:8:1.00:1.00:0.11:0.20), (Cardiac.dysfunction:1:4876:0:8:1.00:1.00:0.11:0.20), (Cardiov ascular.evaluation:1:4876:0:8:1.00:1.00:0.11:0.20), (Chest.X-ray.abnormal:1:4876:0:8:1.00:1.00:0.11:0.20), (Coagulation.factor.V.level.decreased:1:4876:0:8: 1.00:1.00:0.11:0.20), (Colposcopy:1:4876:0:8:1.00:1.00:0.11:0.20), (Complications.of.transplant.s urgery:1:4876:0:8:1.00:1.00:0.11:0.20), (Congenital.mitral.valve.stenosis:1:4876:0:8:1.00:1.00:0. 11:0.20), (Coronary.artery.dissection:1:4876:0:8:1.00:1.00:0.11:0.20), (Dermatofibrosarcoma.prot uberans.metastatic:1:4876:0:8:1.00:1.00:0.11:0.20), (Eosinophil.count.abnormal:1:4876:0:8:1.00: 1.00:0.11:0.20), (Erythema.infectiosum:1:4876:0:8:1.00:1.00:0.11:0.20), (Gastrointestinal.ulcer.m anagement:1:4876:0:8:1.00:1.00:0.11:0.20), (Genital.discomfort:1:4876:0:8:1.00:1.00:0.11:0.20), (Glucocorticoids.abnormal:1:4876:0:8:1.00:1.00:0.11:0.20), (Heavy.chain.disease:1:4876:0:8:1.0 0:1.00:0.11:0.20), (HIV.infection.CDC.Group.I:1:4876:0:8:1.00:1.00:0.11:0.20), (Hyperkeratosis. lenticularis.perstans:1:4876:0:8:1.00:1.00:0.11:0.20), (Hypoplastic.anaemia:1:4876:0:8:1.00:1.00: 0.11:0.20), (Imaging.procedure.abnormal:1:4876:0:8:1.00:1.00:0.11:0.20), (Inappropriate.schedul e.of.drug.administration:1:4876:0:8:1.00:1.00:0.11:0.20), (Inflammation.of.lacrimal.passage:1:48 76:0:8:1.00:1.00:0.11:0.20), (Inguinal.hernia.gangrenous:1:4876:0:8:1.00:1.00:0.11:0.20), (Injecti on.site.injury:1:4876:0:8:1.00:1.00:0.11:0.20), (Intestinal.polyp:1:4876:0:8:1.00:1.00:0.11:0.20), (Joint.lock:1:4876:0:8:1.00:1.00:0.11:0.20), (Kidney.contusion:1:4876:0:8:1.00:1.00:0.11:0.20), (L entigo.maligna:1:4876:0:8:1.00:1.00:0.11:0.20), (Lymphogranuloma.venereum:1:4876:0:8:1.00:1 0.00:0.11:0.20), (Malignant.bowel.obstruction:1:4876:0:8:1.00:1.00:0.11:0.20), (Myoclonic.epileps y.and.ragged-red.fibres:1:4876:0:8:1.00:1.00:0.11:0.20), (N-terminal.prohormone.brain.natriuretic.peptide.increased:1:4876:0:8:1.00:1.00:0.11:0.20), (Ocular. surface.disease:1:4876:0:8:1.00:1.00:0.11:0.20), (Oesophageal.squamous.cell.carcinoma.stage.IV: 1:4876:0:8:1.00:1.00:0.11:0.20), (Organ.donor:1:4876:0:8:1.00:1.00:0.11:0.20), (Osteoporosis.cir cumscripta.cranii:1:4876:0:8:1.00:1.00:0.11:0.20), (Peritoneal.cloudy.effluent:1:4876:0:8:1.00:1. 00:0.11:0.20), (Pinguecula:1:4876:0:8:1.00:1.00:0.11:0.20), (Pituitary.infarction:1:4876:0:8:1.00: 1.00:0.11:0.20), (Post.inflammatory.pigmentation.change:1:4876:0:8:1.00:1.00:0.11:0.20), (Post. procedural.contusion:1:4876:0:8:1.00:1.00:0.11:0.20), (Postictal.paralysis:1:4876:0:8:1.00:1.00:0. 11:0.20), (Pseudofolliculitis.barbae:1:4876:0:8:1.00:1.00:0.11:0.20), (Pseudoxanthoma.elasticum: 1:4876:0:8:1.00:1.00:0.11:0.20), (Pulmonary.capillary.haemangiomatosis:1:4876:0:8:1.00:1.00:0. 11:0.20), (Radiotherapy.to.brain:1:4876:0:8:1.00:1.00:0.11:0.20), (Renal.cell.carcinoma.stage.I:1: 4876:0:8:1.00:1.00:0.11:0.20), (Retinal.melanoma:1:4876:0:8:1.00:1.00:0.11:0.20), (Rhinoplasty: 1:4876:0:8:1.00:1.00:0.11:0.20), (Skull.fracture:1:4876:0:8:1.00:1.00:0.11:0.20), (Sleep.talking:1: 4876:0:8:1.00:1.00:0.11:0.20), (Spastic.paralysis:1:4876:0:8:1.00:1.00:0.11:0.20), (Teratoma.beni gn:1:4876:0:8:1.00:1.00:0.11:0.20), (Tracheal.neoplasm:1:4876:0:8:1.00:1.00:0.11:0.20), (Tumefa ctive.multiple.sclerosis:1:4876:0:8:1.00:1.00:0.11:0.20), (Uterine.inflammation:1:4876:0:8:1.00:1 0.00:0.11:0.20), (Vaginal.pH.abnormal:1:4876:0:8:1.00:1.00:0.11:0.20), (Waldenstrom's.macroglo bulinaemia.stage.IV:1:4876:0:8:1.00:1.00:0.11:0.20), (West.Nile.viral.infection:1:4876:0:8:1.00: 1.00:0.11:0.20), (Abdominoplasty:0:4876:0:9:1.00:#NUM!:0.00:#NUM!), (Accessory.cardiac.pat hway:0:4876:0:9:1.00:#NUM!:0.00:#NUM!), (Acquired.mixed.hyperlipidaemia:0:4876:0:9:1.00: #NUM!:0.00:#NUM!), (Adenine.phosphoribosyl.transferase.deficiency:0:4876:0:9:1.00:#NUM!: 0.00:#NUM!), (Amino.acid.catabolism.disorder:0:4876:0:9:1.00:#NUM!:0.00:#NUM!), (Amygda lotomy:0:4876:0:9:1.00:#NUM!:0.00:#NUM!), (Anal.atresia:0:4876:0:9:1.00:#NUM!:0.00:#NU M!), (Anal.fissure.excision:0:4876:0:9:1.00:#NUM!:0.00:#NUM!), (Angiomyxoma:0:4876:0:9:1. 00:#NUM!:0.00:#NUM!), (Angiotensin.I:0:4876:0:9:1.00:#NUM!:0.00:#NUM!), (Antimicrobial. susceptibility.test:0:4876:0:9:1.00:#NUM!:0.00:#NUM!), (Aortic.embolus:0:4876:0:9:1.00:#NU M!:0.00:#NUM!), (Aorto-duodenal.fistula:0:4876:0:9:1.00:#NUM!:0.00:#NUM!), (Application.site.anaesthesia:0:4876:0:9: 1.00:#NUM!:0.00:#NUM!), (Arrhythmogenic.right.ventricular.dysplasia:0:4876:0:9:1.00:#NUM!: 0.00:#NUM!), (Bacterial.test:0:4876:0:9:1.00:#NUM!:0.00:#NUM!), (Behaviour.disorder.due.to. a.general.medical.condition:0:4876:0:9:1.00:#NUM!:0.00:#NUM!), (Biopsy.thyroid.gland:0:487 6:0:9:1.00:#NUM!:0.00:#NUM!), (Bladder.sphincter.atony:0:4876:0:9:1.00:#NUM!:0.00:#NUM !), (Blood.beta-D-glucan:0:4876:0:9:1.00:#NUM!:0.00:#NUM!), (Blood.ethanol:0:4876:0:9:1.00:#NUM!:0.00:#N UM!), (Blood.lactic.acid.increased:0:4876:0:9:1.00:#NUM!:0.00:#NUM!), (Blood.lead:0:4876:0: 9:1.00:#NUM!:0.00:#NUM!), (Blood.osmolarity.increased:0:4876:0:9:1.00:#NUM!:0.00:#NUM!), (Blood.prolactin:0:4876:0:9:1.00:#NUM!:0.00:#NUM!), (Bone.marrow.toxicity:0:4876:0:9:1.0 0:#NUM!:0.00:#NUM!), (Brachial.plexopathy:0:4876:0:9:1.00:#NUM!:0.00:#NUM!), (Brain.ste m.syndrome:0:4876:0:9:1.00:#NUM!:0.00:#NUM!), (Brow.ptosis:0:4876:0:9:1.00:#NUM!:0.00: #NUM!), (Bursitis.infective.staphylococcal:0:4876:0:9:1.00:#NUM!:0.00:#NUM!), (Byssinosis:0: 4876:0:9:1.00:#NUM!:0.00:#NUM!), (Carbon.dioxide.decreased:0:4876:0:9:1.00:#NUM!:0.00:#NUM!), (Cautery.to.nose:0:4876:0:9:1.00:#NUM!:0.00:#NUM!), (Central.venous.pressure:0:487 6:0:9:1.00:#NUM!:0.00:#NUM!), (Cerebrospinal. fluid. circulation.disorder:0:4876:0:9:1.00:#NU M!:0.00:#NUM!), (Cervix.oedema:0:4876:0:9:1.00:#NUM!:0.00:#NUM!), (Clubbing:0:4876:0:9: 1.00:#NUM!:0.00:#NUM!), (Compulsive.handwashing:0:4876:0:9:1.00:#NUM!:0.00:#NUM!), (Computed.tomographic.colonography:0:4876:0:9:1.00:#NUM!:0.00:#NUM!), (Congenital.neurol ogical.disorder:0:4876:0:9:1.00:#NUM!:0.00:#NUM!), (Cutaneous.leishmaniasis:0:4876:0:9:1.00: #NUM!:0.00:#NUM!), (Dermoid.cyst:0:4876:0:9:1.00:#NUM!:0.00:#NUM!), (Dihydrotestostero ne.level:0:4876:0:9:1.00:#NUM!:0.00:#NUM!), (Embedded. device:0:4876:0:9:1.00:#NUM!:0.00: #NUM!), (Enterostomy:0:4876:0:9:1.00:#NUM!:0.00:#NUM!), (Epididymal.cyst.removal:0:487 6:0:9:1.00:#NUM!:0.00:#NUM!), (Epidural.catheter.placement:0:4876:0:9:1.00:#NUM!:0.00:#N UM!), (Erection.increased:0:4876:0:9:1.00:#NUM!:0.00:#NUM!), (Extremity.contracture:0:4876: 0:9:1.00:#NUM!:0.00:#NUM!), (Eye.contusion:0:4876:0:9:1.00:#NUM!:0.00:#NUM!), (Factor.I X.inhibition:0:4876:0:9:1.00:#NUM!:0.00:#NUM!), (Filariasis:0:4876:0:9:1.00:#NUM!:0.00:#N UM!), (Fms-like.tyrosine.kinase.3. positive:0:4876:0:9:1.00:#NUM!:0.00:#NUM!), (Gastric.haemangioma:0:4 876:0:9:1.00:#NUM!:0.00:#NUM!), (Gastrooesophageal.heterotopia:0:4876:0:9:1.00:#NUM!:0.0 0:#NUM!), (Glaucomatous.optic.disc.atrophy:0:4876:0:9:1.00:#NUM!:0.00:#NUM!), (Gun. shot. wound:0:4876:0:9:1.00:#NUM!:0.00:#NUM!), (Hand-foot-genital.syndrome:0:4876:0:9:1.00:#NUM!:0.00:#NUM!), (Helicobacter.test:0:4876:0:9:1.00:#N UM!:0.00:#NUM!), (Hepatitis.viral.test.positive:0:4876:0:9:1.00:#NUM!:0.00:#NUM!), (Histopla smosis.cutaneous:0:4876:0:9:1.00:#NUM!:0.00:#NUM!), (Hydrocele:0:4876:0:9:1.00:#NUM!:0. 00:#NUM!), (Hypercatabolism:0:4876:0:9:1.00:#NUM!:0.00:#NUM!), (Hyperdynamic.left.ventri cle:0:4876:0:9:1.00:#NUM!:0.00:#NUM!), (IDH.differentiation.syndrome:0:4876:0:9:1.00:#NU M!:0.00:#NUM!), (Inadequate.analgesia:0:4876:0:9:1.00:#NUM!:0.00:#NUM!), (Isoimmune.hae molytic.disease:0:4876:0:9:1.00:#NUM!:0.00:#NUM!), (Klippel-Feil.syndrome:0:4876:0:9:1.00:#NUM!:0.00:#NUM!), (Laryngomalacia:0:4876:0:9:1.00:#NUM!: 0.00:#NUM!), (Lymphoma.cutis:0:4876:0:9:1.00:#NUM!:0.00:#NUM!), (Medical.device.site.sw elling:0:4876:0:9:1.00:#NUM!:0.00:#NUM!), (Metachromatic.leukodystrophy:0:4876:0:9:1.00:#NUM!:0.00:#NUM!), (Metamorphopsia:0:4876:0:9:1.00:#NUM!:0.00:#NUM!), (Monoclonal.im munoglobulin.present:0:4876:0:9:1.00:#NUM!:0.00:#NUM!), (Mucinous.cystadenocarcinoma.ov ary:0:4876:0:9:1.00:#NUM!:0.00:#NUM!), (Neck. deformity:0:4876:0:9:1.00:#NUM!:0.00:#NU M!), (Neurological.examination:0:4876:0:9:1.00:#NUM!:0.00:#NUM!), (Neuropathy.vitamin.B6. deficiency:0:4876:0:9:1.00:#NUM!:0.00:#NUM!), (Nodal.marginal.zone.B-cell.lymphoma.refractory:0:4876:0:9:1.00:#NUM!:0.00:#NUM!), (Oesophageal.dilation.procedur e:0:4876:0:9:1.00:#NUM!:0.00:#NUM!), (Oesophageal.variceal.ligation:0:4876:0:9:1.00:#NUM!: 0.00:#NUM!), (Oropharyngeal.cancer.stage.IV:0:4876:0:9:1.00:#NUM!:0.00:#NUM!), (Pericardi al.disease:0:4876:0:9:1.00:#NUM!:0.00:#NUM!), (Periorbital.abscess:0:4876:0:9:1.00:#NUM!:0. 00:#NUM!), (Peripheral.nerve.transposition:0:4876:0:9:1.00:#NUM!:0.00:#NUM!), (Persistent.cl oaca:0:4876:0:9:1.00:#NUM!:0.00:#NUM!), (Platelet.adhesiveness:0:4876:0:9:1.00:#NUM!:0.00: #NUM!), (Portal.shunt.procedure:0:4876:0:9:1.00:#NUM!:0.00:#NUM!), (Prodromal.Alzheimer's. disease:0:4876:0:9:1.00:#NUM!:0.00:#NUM!), (Product.odour.abnormal:0:4876:0:9:1.00:#NU M!:0.00:#NUM!), (Pump.reservoir.issue:0:4876:0:9:1.00:#NUM!:0.00:#NUM!), (Radical.hystere ctomy:0:4876:0:9:1.00:#NUM!:0.00:#NUM!), (Radiotherapy.to.lung:0:4876:0:9:1.00:#NUM!:0. 00:#NUM!), (Rapid.correction.of.hyponatraemia:0:4876:0:9:1.00:#NUM!:0.00:#NUM!), (Rectoc ele.repair:0:4876:0:9:1.00:#NUM!:0.00:#NUM!), (Renal.function.test.abnormal:0:4876:0:9:1.00: #NUM!:0.00:#NUM!), (Retinopexy:0:4876:0:9:1.00:#NUM!:0.00:#NUM!), (Salpingo-oophorectomy:0:4876:0:9:1.00:#NUM!:0.00:#NUM!), (Schizophrenia.simple:0:4876:0:9:1.00:#NUM!:0.00:#NUM!), (Solar.urticaria:0:4876:0:9:1.00:#NUM!:0.00:#NUM!), (Spinal.flattening:0: 4876:0:9:1.00:#NUM!:0.00:#NUM!), (Stem.cell.donor:0:4876:0:9:1.00:#NUM!:0.00:#NUM!), (S ticky.skin:0:4876:0:9:1.00:#NUM!:0.00:#NUM!), (Stoma.site.discomfort:0:4876:0:9:1.00:#NUM !:0.00:#NUM!), (Suture.rupture:0:4876:0:9:1.00:#NUM!:0.00:#NUM!), (Thyroid.function.test:0:4 876:0:9:1.00:#NUM!:0.00:#NUM!), (Thyroid.malformation:0:4876:0:9:1.00:#NUM!:0.00:#NU M!), (Tissue.expansion.procedure:0:4876:0:9:1.00:#NUM!:0.00:#NUM!), (Tooth.deposit:0:4876: 0:9:1.00:#NUM!:0.00:#NUM!), (Trichiasis:0:4876:0:9:1.00:#NUM!:0.00:#NUM!), (Tularaemia:0: 4876:0:9:1.00:#NUM!:0.00:#NUM!), (Tunnel.vision:0:4876:0:9:1.00:#NUM!:0.00:#NUM!), (Ult rasound.liver:0:4876:0:9:1.00:#NUM!:0.00:#NUM!), (Urethral.atrophy:0:4876:0:9:1.00:#NUM!: 0.00:#NUM!), (Urethral.repair:0:4876:0:9:1.00:#NUM!:0.00:#NUM!), (Urine.osmolarity.decreas ed:0:4876:0:9:1.00:#NUM!:0.00:#NUM!), (Vaccine.breakthrough.infection:0:4876:0:9:1.00:#NU M!:0.00:#NUM!), (Vascular.wall.hypertrophy:0:4876:0:9:1.00:#NUM!:0.00:#NUM!), (Ventricula r.dyskinesia:0:4876:0:9:1.00:#NUM!:0.00:#NUM!), (X-ray.gastrointestinal.tract.normal:0:4876:0:9:1.00:#NUM!:0.00:#NUM!), (Mikulicz's.syndrome:15:4861:0:9:1.00:1.00:0.63:0.77), (HIV.infection.WHO.clinical.stage.III:13:4863:0:9:1.00:1.00:0.5 9:0.74), (Vanishing.bile.duct.syndrome:9:4867:0:9:1.00:1.00:0.50:0.67), (Alveolar.proteinosis:7:4 869:0:9:1.00:1.00:0.44:0.61), (Segmented.hyalinising.vasculitis:6:4870:0:9:1.00:1.00:0.40:0.57), (Blood.immunoglobulin.M.increased:5:4871:0:9:1.00:1.00:0.36:0.53), (Cytomegalovirus.gastritis: 5:4871:0:9:1.00:1.00:0.36:0.53), (Engraft.failure:5:4871:0:9:1.00:1.00:0.36:0.53), (Acrochordon:4: 4872:0:9:1.00:1.00:0.31:0.47), (Dowling-Degos.disease:4:4872:0:9:1.00:1.00:0.31:0.47), (Encephalitis.brain.stem:4:4872:1:8:1.00:0.80:0.3 3:0.47), (Hodgkin's.disease.lymphocyte.depletion.stage.II.site.unspecified:4:4872:0:9:1.00:1.00:0 0.31:0.47), (Influenza.A.virus.test:4:4872:0:9:1.00:1.00:0.31:0.47), (Metastases.to.heart:4:4872:0:9: 1.00:1.00:0.31:0.47), (Ovarian.endometrioid.carcinoma:4:4872:0:9:1.00:1.00:0.31:0.47), (Respira tory.muscle.weakness:4:4872:0:9:1.00:1.00:0.31:0.47), (Accidental.poisoning:3:4873:0:9:1.00:1. 00:0.25:0.40), (Anorectal.swelling:3:4873:0:9:1.00:1.00:0.25:0.40), (Aortic.valvotomy:3:4873:0:9: 1.00:1.00:0.25:0.40), (Cardiac.neoplasm.unspecified:3:4873:0:9:1.00:1.00:0.25:0.40), (Chronic.a ctinic.dermatitis:3:4873:0:9:1.00:1.00:0.25:0.40), (Gastric.cancer.stage.0:3:4873:0:9:1.00:1.00:0. 25:0.40), (Hyperammonaemic.encephalopathy:3:4873:0:9:1.00:1.00:0.25:0.40), (Moyamoya.disea se:3:4873:0:9:1.00:1.00:0.25:0.40), (Nuclear.magnetic.resonance.imaging.renal:3:4873:0:9:1.00: 1.00:0.25:0.40), (Rubella:3:4873:0:9:1.00:1.00:0.25:0.40), (Acute.polyneuropathy:2:4874:0:9:1.0 0:1.00:0.18:0.31), (Anti.factor.VIII.antibody.increased:2:4874:0:9:1.00:1.00:0.18:0.31), (Aplasia. cutis.congenita:2:4874:0:9:1.00:1.00:0.18:0.31), (Areflexia:2:4874:0:9:1.00:1.00:0.18:0.31), (Atri oventricular.septal.defect:2:4874:0:9:1.00:1.00:0.18:0.31), (Blood.prolactin.decreased:2:4874:0:9: 1.00:1.00:0.18:0.31), (Cerebral.hyperperfusion.syndrome:2:4874:0:9:1.00:1.00:0.18:0.31), (Cong enital.central.nervous.system.anomaly:2:4874:0:9:1.00:1.00:0.18:0.31), (Corneal.defect:2:4874:0: 9:1.00:1.00:0.18:0.31), (Delayed.effects.of.radiation:2:4874:0:9:1.00:1.00:0.18:0.31), (Drug.tolera nce.decreased:2:4874:0:9:1.00:1.00:0.18:0.31), (Erythroleukaemia:2:4874:0:9:1.00:1.00:0.18:0.3 1), (Foetal-matemal.haemorrhage:2:4874:0:9:1.00:1.00:0.18:0.31), (Infection.reactivation:2:4874:0:9:1.00:1. 00:0.18:0.31), (Isosporiasis:2:4874:0:9:1.00:1.00:0.18:0.31), (Laryngeal.inflammation:2:4874:0:9: 1.00:1.00:0.18:0.31), (Listeria.encephalitis:2:4874:0:9:1.00:1.00:0.18:0.31), (Mastoid.disorder:2:4 874:0:9:1.00:1.00:0.18:0.31), (Menstruation.normal:2:4874:0:9:1.00:1.00:0.18:0.31), (Mesenteric. abscess:2:4874:0:9:1.00:1.00:0.18:0.31), (Methaemoglobinaemia:2:4874:0:9:1.00:1.00:0.18:0.31), (Optic.disc.disorder:2:4874:0:9:1.00:1.00:0.18:0.31), (Peritoneal.haemorrhage:2:4874:0:9:1.00:1 0.00:0.18:0.31), (Pharyngeal.cancer.metastatic:2:4874:0:9:1.00:1.00:0.18:0.31), (Plantar.fascial.fibr omatosis:2:4874:0:9:1.00:1.00:0.18:0.31), (Pneumonia.31), (Pneumonia.herpes.viral:2:4874:0:9:1.00:1.00:0.18:0.31), (Scar.excision:2:4874:0:9:1.00:1.0 0:0.18:0.31), (Social.alcohol.drinker:2:4874:0:9:1.00:1.00:0.18:0.31), (Stasis.syndrome:2:4874:0: 9:1.00:1.00:0.18:0.31), (Ulnar.neuritis:2:4874:0:9:1.00:1.00:0.18:0.31), (Venous.pressure:2:4874: 0:9:1.00:1.00:0.18:0.31), (Vulvar.adenocarcinoma:2:4874:0:9:1.00:1.00:0.18:0.31), (Abscess.fung al:1:4875:0:9:1.00:1.00:0.10:0.18), (Aged.parent:1:4875:0:9:1.00:1.00:0.10:0.18), (Analgesic.dru g.level.above.therapeutic:1:4875:0:9:1.00:1.00:0.10:0.18), (Angiotensin.converting.enzyme.inhib itor.foetopathy:1:4875:0:9:1.00:1.00:0.10:0.18), (Anterior.chamber.cleavage.syndrome:1:4875:0: 9:1.00:1.00:0.10:0.18), (Arteriovenous.fistula:1:4875:0:9:1.00:1.00:0.10:0.18), (Artery.dissection: 1:4875:0:9:1.00:1.00:0.10:0.18), (Autoimmune.thyroid.disorder:1:4875:0:9:1.00:1.00:0.10:0.18), (Blood.copper:1:4875:0:9:1.00:1.00:0.10:0.18), (Brachytherapy.to.prostate:1:4875:0:9:1.00:1.00:0 0.10:0.18), (Cerebral.haemangioma:1:4875:0:9:1.00:1.00:0.10:0.18), (Cerebrohepatorenal.syndrom e:1:4875:0:9:1.00:1.00:0.10:0.18), (Chlamydia.test.positive:1:4875:0:9:1.00:1.00:0.10:0.18), (Cho lecystostomy:1:4875:0:9:1.00:1.00:0.10:0.18), (Delusion.of.grandeur:1:4875:0:9:1.00:1.00:0.10:0 0.18), (Familial.amyloidosis:1:4875:0:9:1.00:1.00:0.10:0.18), (Gastrooesophageal.sphincter.insuffi ciency:1:4875:0:9:1.00:1.00:0.10:0.18), (Hereditary.haemolytic.anaemia:1:4875:0:9:1.00:1.00:0. 10:0.18), (Herpes.simplex.gastritis:1:4875:0:9:1.00:1.00:0.10:0.18), (HIV.infection.CDC.Group.II: 1:4875:0:9:1.00:1.00:0.10:0.18), (Home.care:1:4875:0:9:1.00:1.00:0.10:0.18), (Immunisation.rea ction:1:4875:0:9:1.00:1.00:0.10:0.18), (Inborn.error.of.lipid.metabolism:1:4875:0:9:1.00:1.00:0.1 0:0.18), (Intramedullary.rod.insertion:1:4875:0:9:1.00:1.00:0.10:0.18), (Left.atrial.appendage.occl usion:1:4875:0:9:1.00:1.00:0.10:0.18), (Localised.cooling.therapy:1:4875:0:9:1.00:1.00:0.10:0.18), (Mononucleosis.syndrome:1:4875:0:9:1.00:1.00:0.10:0.18), (Nerve.root.injury.lumbar:1:4875:0: 9:1.00:1.00:0.10:0.18), (Oesophageal.mass:1:4875:0:9:1.00:1.00:0.10:0.18), (Optic.nerve.cuppin g:1:4875:0:9:1.00:1.00:0.10:0.18), (Optic.nerve.neoplasm:1:4875:0:9:1.00:1.00:0.10:0.18), (Pelvi c.congestion:1:4875:0:9:1.00:1.00:0.10:0.18), (Peripheral.nerve.decompression:1:4875:0:9:1.00:1 0.00:0.10:0.18), (Pyogenic.granuloma:1:4875:0:9:1.00:1.00:0.10:0.18), (Pythium.insidiosum.infecti on:1:4875:0:9:1.00:1.00:0.10:0.18), (Rash.morbilliform:1:4875:0:9:1.00:1.00:0.10:0.18), (Reactio n.to.drug.excipients:1:4875:0:9:1.00:1.00:0.10:0.18), (Riley-Day.syndrome:1:4875:0:9:1.00:1.00:0.10:0.18), (Septo-optic.dysplasia:1:4875:0:9:1.00:1.00:0.10:0.18), (Sick.building.syndrome:1:4875:0:9:1.00:1.00:0. 10:0.18), (Sight.disability:1:4875:0:9:1.00:1.00:0.10:0.18), (Skin.lesion.excision:1:4875:0:9:1.00: 1.00:0.10:0.18), (Tonsillar.inflammation:1:4875:0:9:1.00:1.00:0.10:0.18), (Tracheal.haemorrhage: 1:4875:0:9:1.00:1.00:0.10:0.18), (Tropical.spastic.paresis:1:4875:0:9:1.00:1.00:0.10:0.18), (Tropo nin:1:4875:0:9:1.00:1.00:0.10:0.18), (Urological.examination.abnormal:1:4875:0:9:1.00:1.00:0.1 0:0.18), (Vascular.stent.restenosis:1:4875:0:9:1.00:1.00:0.10:0.18), (Victim.of.crime:1:4875:0:9:1 0.00:1.00:0.10:0.18), (Walking.aid.user:1:4875:0:9:1.00:1.00:0.10:0.18), (Acinetobacter.bacteraemi a:0:4875:0:10:1.00:#NUM!:0.00:#NUM!), (Angiokeratoma:0:4875:0:10:1.00:#NUM!:0.00:#NU M!), (Anticonvulsant.drug.level.above.therapeutic:0:4875:0:10:1.00:#NUM!:0.00:#NUM!), (Apra xia:0:4875:0:10:1.00:#NUM!:0.00:#NUM!), (Arterial.restenosis:0:4875:0:10:1.00:#NUM!:0.00:#NUM!), (Aspergillus.test.negative:0:4875:0:10:1.00:#NUM!:0.00:#NUM!), (Avoidant.personality.disorder:0:4875:0:10:1.00:#NUM!:0.00:#NUM!), (Bereavement:0:4875:0:10:1.00:#NUM!:0.00: #NUM!), (Blood.acid.phosphatase.increased:0:4875:0:10:1.00:#NUM!:0.00:#NUM!), (Blood.lact ic.acid.abnormal:0:4875:0:10:1.00:#NUM!:0.00:#NUM!), (Blood.testosterone.normal:0:4875:0:1 0:1.00:#NUM!:0.00:#NUM!), (Blue.toe.syndrome:0:4875:0:10:1.00:#NUM!:0.00:#NUM!), (Brai n.teratoma:0:4875:0:10:1.00:#NUM!:0.00:#NUM!), (Breast.ulceration:0:4875:0:10:1.00:#NUM!: 0.00:#NUM!), (Bunion.operation:0:4875:0:10:1.00:#NUM!:0.00:#NUM!), (Bursa.injury:0:4875:0: 10:1.00:#NUM!:0.00:#NUM!), (Cardiomyopathy.alcoholic:0:4875:0:10:1.00:#NUM!:0.00:#NU M!), (Cholesterol.absorption.efficiency.decreased:0:4875:0:10:1.00:#NUM!:0.00:#NUM!), (Coag ulation.factor.VIII.level.abnormal:0:4875:0:10:1.00:#NUM!:0.00:#NUM!), (Comeal.decompensa tion:0:4875:0:10:1.00:#NUM!:0.00:#NUM!), (Cotard's.syndrome:0:4875:0:10:1.00:#NUM!:0.00: #NUM!), (Cystic.fibrosis.respiratory.infection.suppression:0:4875:0:10:1.00:#NUM!:0.00:#NUM !), (Cystocele:0:4875:0:10:1.00:#NUM!:0.00:#NUM!), (Cystocele.repair:0:4875:0:10:1.00:#NUM !:0.00:#NUM!), (Dental.prosthesis.user:0:4875:0:10:1.00:#NUM!:0.00:#NUM!), (Dermatofibrosa rcoma.protuberans:0:4875:0:10:1.00:#NUM!:0.00:#NUM!), (Diabetic.microangiopathy:0:4875:0: 10:1.00:#NUM!:0.00:#NUM!), (Diabetic.neuropathic.ulcer:0:4875:0:10:1.00:#NUM!:0.00:#NU M!), (Discogram:0:4875:0:10:1.00:#NUM!:0.00:#NUM!), (Duodenal.switch:0:4875:0:10:1.00:#N UM!:0.00:#NUM!), (Endocarditis.noninfective:0:4875:0:10:1.00:#NUM!:0.00:#NUM!), (Eosinop hilic.cellulitis:0:4875:0:10:1.00:#NUM!:0.00:#NUM!), (Ex-alcoholic:0:4875:0:10:1.00:#NUM!:0.00:#NUM!), (Exposure.to.allergen:0:4875:0:10:1.00:#NU M!:0.00:#NUM!), (Fistulogram:0:4875:0:10:1.00:#NUM!:0.00:#NUM!), (Gamma.radiation.thera py.to.brain:0:4875:0:10:1.00:#NUM!:0.00:#NUM!), (Gastric.stenosis:0:4875:0:10:1.00:#NUM!:0 0.00:#NUM!), (Grandiosity:0:4875:0:10:1.00:#NUM!:0.00:#NUM!), (Hand-arm.vibration.syndrome:0:4875:0:10:1.00:#NUM!:0.00:#NUM!), (Hepatitis.B.DNA.assay.positiv e:0:4875:0:10:1.00:#NUM!:0.00:#NUM!), (Hodgkin's.disease.nodular.sclerosis.stage.I:0:4875:0: 10:1.00:#NUM!:0.00:#NUM!), (Hyperamylasaemia:0:4875:0:10:1.00:#NUM!:0.00:#NUM!), (Hy potony.of.eye:0:4875:0:10:1.00:#NUM!:0.00:#NUM!), (Implant.site.abscess:0:4875:0:10:1.00:#NUM!:0.00:#NUM!), (Implant.site.infection:0:4875:0:10:1.00:#NUM!:0.00:#NUM!), (Infantile.g enetic.agranulocytosis:0:4875:0:10:1.00:#NUM!:0.00:#NUM!), (Infected.bunion:0:4875:0:10:1.0 0:#NUM!:0.00:#NUM!), (Injection.site.phlebitis:0:4875:0:10:1.00:#NUM!:0.00:#NUM!), (Iridoto my:0:4875:0:10:1.00:#NUM!:0.00:#NUM!), (Keratitis.interstitial:0:4875:0:10:1.00:#NUM!:0.00: #NUM!), (Laryngectomy:0:4875:0:10:1.00:#NUM!:0.00:#NUM!), (Laryngoscopy:0:4875:0:10:1. 00:#NUM!:0.00:#NUM!), (Laziness:0:4875:0:10:1.00:#NUM!:0.00:#NUM!), (Lymph.gland.infec tion:0:4875:0:10:1.00:#NUM!:0.00:#NUM!), (Measles.immunisation:0:4875:0:10:1.00:#NUM!:0 0.00:#NUM!), (Meigs′.syndrome:0:4875:0:10:1.00:#NUM!:0.00:#NUM!), (Muscle.hypertrophy:0: 4875:0:10:1.00:#NUM!:0.00:#NUM!), (Myocardial.rupture:0:4875:0:10:1.00:#NUM!:0.00:#NU M!), (Nail.operation:0:4875:0:10:1.00:#NUM!:0.00:#NUM!), (Needle.issue:0:4875:0:10:1.00:#N UM!:0.00:#NUM!), (Nematodiasis:0:4875:0:10:1.00:#NUM!:0.00:#NUM!), (Neonatal.hypoxia:0: 4875:0:10:1.00:#NUM!:0.00:#NUM!), (Nipple.disorder:0:4875:0:10:1.00:#NUM!:0.00:#NUM!), (Non-infectious.endophthalmitis:0:4875:0:10:1.00:#NUM!:0.00:#NUM!), (Occult.blood:0:4875:0:10:1. 00:#NUM!:0.00:#NUM!), (Oesophageal.mucosal.hyperplasia:0:4875:0:10:1.00:#NUM!:0.00:#N UM!), (Oral.papilloma:0:4875:0:10:1.00:#NUM!:0.00:#NUM!), (Oropharyngeal.surgery:0:4875:0: 10:1.00:#NUM!:0.00:#NUM!), (Oxygen.saturation:0:4875:0:10:1.00:#NUM!:0.00:#NUM!), (Pan tothenic.acid.deficiency:0:4875:0:10:1.00:#NUM!:0.00:#NUM!), (Pelvic.exenteration:0:4875:0:1 0:1.00:#NUM!:0.00:#NUM!), (Pelvic.floor.dyssynergia:0:4875:0:10:1.00:#NUM!:0.00:#NUM!), (Pelvic.haematoma:0:4875:0:10:1.00:#NUM!:0.00:#NUM!), (Perineal.injury:0:4875:0:10:1.00:#NUM!:0.00:#NUM!), (Peripheral.nerve.operation:0:4875:0:10:1.00:#NUM!:0.00:#NUM!), (Pituit ary.haemorrhage:0:4875:0:10:1.00:#NUM!:0.00:#NUM!), (Platelet.aggregation.decreased:0:4875: 0:10:1.00:#NUM!:0.00:#NUM!), (Prothrombin.time:0:4875:0:10:1.00:#NUM!:0.00:#NUM!), (P ulse.absent:0:4875:0:10:1.00:#NUM!:0.00:#NUM!), (Reactive.psychosis:0:4875:0:10:1.00:#NU M!:0.00:#NUM!), (Salpingitis:0:4875:0:10:1.00:#NUM!:0.00:#NUM!), (Severe.asthma.with. fung al.sensitisation:0:4875:0:10:1.00:#NUM!:0.00:#NUM!), (Severe.mental.retardation:0:4875:0:10: 1.00:#NUM!:0.00:#NUM!), (Splenic.neoplasm.malignancy.unspecified:0:4875:0:10:1.00:#NUM !:0.00:#NUM!), (Thyroid. size.decreased:0:4875:0:10:1.00:#NUM!:0.00:#NUM!), (Tooth.impacte d:0:4875:0:10:1.00:#NUM!:0.00:#NUM!), (Tri-iodothyronine.uptake:0:4875:0:10:1.00:#NUM!:0.00:#NUM!), (Tricuspid.valve.repair:0:4875:0:1 0:1.00:#NUM!:0.00:#NUM!), (Urea.cycle.disorder:0:4875:0:10:1.00:#NUM!:0.00:#NUM!), (Uret eritis:0:4875:0:10:1.00:#NUM!:0.00:#NUM!), (Urticaria.aquagenic:0:4875:0:10:1.00:#NUM!:0.0 0:#NUM!), (Urticaria.pigmentosa:0:4875:0:10:1.00:#NUM!:0.00:#NUM!), (Varicocele:0:4875:0: 10:1.00:#NUM!:0.00:#NUM!), (Varicophlebitis:0:4875:0:10:1.00:#NUM!:0.00:#NUM!), (Vein.r upture:0:4875:0:10:1.00:#NUM!:0.00:#NUM!), (Venomous.bite:0:4875:0:10:1.00:#NUM!:0.00: #NUM!), (Vital.capacity.decreased:0:4875:0:10:1.00:#NUM!:0.00:#NUM!), (Vulvovaginal.infla mmation:0:4875:0:10:1.00:#NUM!:0.00:#NUM!), (Vulvovaginitis.trichomonal:0:4875:0:10:1.00: #NUM!:0.00:#NUM!), (X-ray.with.contrast.lower.gastrointestinal.tract:0:4875:0:10:1.00:#NUM!:0.00:#NUM!), (Intestine.tr ansplant.rejection:11:4864:0:10:1.00:1.00:0.52:0.69), (Oocyte.harvest:9:4866:1:9:1.00:0.90:0.50: 0.64), (Sinus.cancer.metastatic:9:4866:0:10:1.00:1.00:0.47:0.64), (Idiopathic.pneumonia.syndrom e:7:4868:0:10:1.00:1.00:0.41:0.58), (Signet-ring.cell.carcinoma:7:4868:0:10:1.00:1.00:0.41:0.58), (Metastatic.glioma:6:4869:0:10:1.00:1.00: 0.38:0.55), (Neck.exploration:6:4869:0:10:1.00:1.00:0.38:0.55), (Venous.stent.insertion:6:4869:0: 10:1.00:1.00:0.38:0.55), (Benign.hydatidiform.mole:5:4870:0:10:1.00:1.00:0.33:0.50), (Colorectal.cancer.stage.II:5:4870:0:10:1.00:1.00:0.33:0.50), (Hepatic.vein.thrombosis:5:4870:0:10:1.00:1.0 0:0.33:0.50), (Maternal.exposure.before.pregnancy:5:4870:0:10:1.00:1.00:0.33:0.50), (Metastases. to.abdominal.wall:5:4870:0:10:1.00:1.00:0.33:0.50), (Microangiopathic.haemolytic.anaemia:5:48 70:0:10:1.00:1.00:0.33:0.50), (Osteopathic.treatment:5:4870:0:10:1.00:1.00:0.33:0.50), (Acinar.ce 11.carcinoma.of.pancreas:4:4871:0:10:1.00:1.00:0.29:0.44), (Albright's.disease:4:4871:0:10:1.00:1 0.00:0.29:0.44), (Cerebral.sarcoidosis:4:4871:0:10:1.00:1.00:0.29:0.44), (Insulin.resistance.syndro me:4:4871:0:10:1.00:1.00:0.29:0.44), (Lupus.encephalitis:4:4871:0:10:1.00:1.00:0.29:0.44), (Para doxical.embolism:4:4871:0:10:1.00:1.00:0.29:0.44), (Vital.dye.staining.cornea.present:4:4871:0: 10:1.00:1.00:0.29:0.44), (Ataxia.telangiectasia:3:4872:0:10:1.00:1.00:0.23:0.38), (Bone.scan.abno rmal:3:4872:0:10:1.00:1.00:0.23:0.38), (Granulicatella.infection:3:4872:0:10:1.00:1.00:0.23:0.38), (Leriche.syndrome:3:4872:0:10:1.00:1.00:0.23:0.38), (Liver.function.test:3:4872:0:10:1.00:1.00: 0.23:0.38), (Lupus.pneumonitis:3:4872:0:10:1.00:1.00:0.23:0.38), (Metastases.to.spleen:3:4872:0: 10:1.00:1.00:0.23:0.38), (Ovarian.cyst.ruptured:3:4872:0:10:1.00:1.00:0.23:0.38), (Placenta.accre ta:3:4872:0:10:1.00:1.00:0.23:0.38), (Pneumonia.measles:3:4872:0:10:1.00:1.00:0.23:0.38), (Posti ctal.psychosis:3:4872:0:10:1.00:1.00:0.23:0.38), (Thyroid.dysfunction.in.pregnancy:3:4872:0:10: 1.00:1.00:0.23:0.38), (Urticaria.pressure:3:4872:0:10:1.00:1.00:0.23:0.38), (Anal.cancer.stage.II:2: 4873:0:10:1.00:1.00:0.17:0.29), (Benign.neoplasm.of.skin:2:4873:0:10:1.00:1.00:0.17:0.29), (Bili ary.ascites:2:4873:0:10:1.00:1.00:0.17:0.29), (Biopsy.heart:2:4873:0:10:1.00:1.00:0.17:0.29), (Br ain.tumour.operation:2:4873:0:10:1.00:1.00:0.17:0.29), (Cutis.29), (Genital.disorder.female:2:4873:0:10:1.00:1.00:0.17:0.29), (Haematocrit.increased:2:4873:0: 10:1.00:1.00:0.17:0.29), (Hemianaesthesia:2:4873:0:10:1.00:1.00:0.17:0.29), (Hypocapnia:2:4873: 0:10:1.00:1.00:0.17:0.29), (Joint.crepitation:2:4873:0:10:1.00:1.00:0.17:0.29), (Mesenteritis:2:48 73:0:10:1.00:1.00:0.17:0.29), (Neonatal.candida.infection:2:4873:0:10:1.00:1.00:0.17:0.29), (Oral.neoplasm.benign:2:4873:0:10:1.00:1.00:0.17:0.29), (Ovarian.mass:2:4873:0:10:1.00:1.00:0.17:0. 29), (Precursor.T-lymphoblastic.lymphoma/leukaemia.stage.IV:2:4873:0:10:1.00:1.00:0.17:0.29), (Respirovirus.tes t.positive:2:4873:0:10:1.00:1.00:0.17:0.29), (Scan.with.contrast.abnormal:2:4873:0:10:1.00:1.00: 0.17:0.29), (Trigeminal.neuritis:2:4873:0:10:1.00:1.00:0.17:0.29), (Urinary.tract.infection.neonata 1:2:4873:0:10:1.00:1.00:0.17:0.29), (Urticaria.thermal:2:4873:0:10:1.00:1.00:0.17:0.29), (Uterine. cervical.laceration:2:4873:0:10:1.00:1.00:0.17:0.29), (Vein.collapse:2:4873:0:10:1.00:1.00:0.17:0 0.29), (Vulval.cancer.recurrent:2:4873:0:10:1.00:1.00:0.17:0.29), (Acute.chest.syndrome:1:4874:0: 10:1.00:1.00:0.09:0.17), (Adult.T-cell.lymphoma/leukaemia.recurrent:1:4874:0:10:1.00:1.00:0.09:0.17), (Adult.T-cell.lymphoma/leukaemia.refractory:1:4874:0:10:1.00:1.00:0.09:0.17), (Aquagenic.pruritus:1:487 4:0:10:1.00:1.00:0.09:0.17), (Aspartate.aminotransferase:1:4874:0:10:1.00:1.00:0.09:0.17), (Basil ar.artery.stenosis:1:4874:0:10:1.00:1.00:0.09:0.17), (Benign.breast.neoplasm:1:4874:0:10:1.00:1. 00:0.09:0.17), (Blood.bilirubin.abnormal:1:4874:0:10:1.00:1.00:0.09:0.17), (Blood.potassium.nor mal:1:4874:0:10:1.00:1.00:0.09:0.17), (Cerebral.cyst:1:4874:0:10:1.00:1.00:0.09:0.17), (Clumsine ss:1:4874:0:10:1.00:1.00:0.09:0.17), (Confabulation:1:4874:0:10:1.00:1.00:0.09:0.17), (Eyelid.ski n.dryness:1:4874:0:10:1.00:1.00:0.09:0.17), (Fanconi.syndrome:1:4874:0:10:1.00:1.00:0.09:0.17), (Gastric.hypermotility:1:4874:0:10:1.00:1.00:0.09:0.17), (Gastrointestinal.submucosal.tumour:1: 4874:0:10:1.00:1.00:0.09:0.17), (Genital.disorder.male:1:4874:0:10:1.00:1.00:0.09:0.17), (Grip.str ength.decreased:1:4874:0:10:1.00:1.00:0.09:0.17), (Haemoperfusion:1:4874:0:10:1.00:1.00:0.09: 0.17), (Human.bocavirus.infection:1:4874:0:10:1.00:1.00:0.09:0.17), (Infected.bites:1:4874:0:10: 1.00:1.00:0.09:0.17), (Injury.associated.with.device:1:4874:0:10:1.00:1.00:0.09:0.17), (Keratoplas ty:1:4874:0:10:1.00:1.00:0.09:0.17), (Laboratory.test.normal:1:4874:0:10:1.00:1.00:0.09:0.17), (Mass.excision:1:4874:0:10:1.00:1.00:0.09:0.17), (Milia:1:4874:0:10:1.00:1.00:0.09:0.17), (Nervo us.system.neoplasm:1:4874:0:10:1.00:1.00:0.09:0.17), (Pacemaker.syndrome:1:4874:0:10:1.00:1. 00:0.09:0.17), (PCO2.decreased:1:4874:0:10:1.00:1.00:0.09:0.17), (Pertussis.immunisation:1:487 4:0:10:1.00:1.00:0.09:0.17), (Pharyngeal.operation:1:4874:0:10:1.00:1.00:0.09:0.17), (Pituitary.hy poplasia:1:4874:0:10:1.00:1.00:0.09:0.17), (Psychosexual.disorder:1:4874:0:10:1.00:1.00:0.09:0. 17), (Radioembolisation:1:4874:0:10:1.00:1.00:0.09:0.17), (Sclerema:1:4874:0:10:1.00:1.00:0.09: 0.17), (Shunt.malfunction:1:4874:0:10:1.00:1.00:0.09:0.17), (Skin.mass:1:4874:0:10:1.00:1.00:0. 09:0.17), (Spontaneous.haemorrhage:1:4874:0:10:1.00:1.00:0.09:0.17), (Sturge-Weber.syndrome:1:4874:0:10:1.00:1.00:0.09:0.17), (Suicide.of.relative:1:4874:0:10:1.00:1.00:0. 09:0.17), (Tobacco.interaction:1:4874:0:10:1.00:1.00:0.09:0.17), (Ultrasound.Doppler.abnormal:1: 4874:0:10:1.00:1.00:0.09:0.17), (Urine.uric.acid.abnormal:1:4874:0:10:1.00:1.00:0.09:0.17), (Alc aligenes.infection:0:4874:0:11:1.00:#NUM!:0.00:#NUM!), (Ameloblastoma:0:4874:0:11:1.00:#NUM!:0.00:#NUM!), (Amino.acid.level.increased:0:4874:0:11:1.00:#NUM!:0.00:#NUM!), (Angi osarcoma.recurrent:0:4874:0:11:1.00:#NUM!:0.00:#NUM!), (Anorgasmia:0:4874:0:11:1.00:#NU M!:0.00:#NUM!), (Application.site.pruritus:0:4874:0:11:1.00:#NUM!:0.00:#NUM!), (Bile.duct.c ancer.stage.IV:0:4874:0:11:1.00:#NUM!:0.00:#NUM!), (Blood.osmolarity.abnormal:0:4874:0:11: 1.00:#NUM!:0.00:#NUM!), (Blood.pressure.ambulatory:0:4874:0:11:1.00:#NUM!:0.00:#NUM!), (Blunted. affect:0:4874:0:11:1.00:#NUM!:0.00:#NUM!), (Bordetella.infection:0:4874:0:11:1.00: #NUM!:0.00:#NUM!), (Cerebral.endovascular.aneurysm.repair:0:4874:0:11:1.00:#NUM!:0.00:#NUM!), (Cholangiosarcoma:0:4874:0:11:1.00:#NUM!:0.00:#NUM!), (Colon.adenoma:0:4874:0:1 1:1.00:#NUM!:0.00:#NUM!), (Congenital.bronchiectasis:0:4874:0:11:1.00:#NUM!:0.00:#NUM!), (Congenital.bronchomalacia:0:4874:0:11:1.00:#NUM!:0.00:#NUM!), (Congenital.mitochondria 1.cytopathy:0:4874:0:11:1.00:#NUM!:0.00:#NUM!), (Congenital.toxoplasmosis:0:4874:0:11:1.00: #NUM!:0.00:#NUM!), (Coprolalia:0:4874:0:11:1.00:#NUM!:0.00:#NUM!), (CSF.pressure.abnor mal:0:4874:0:11:1.00:#NUM!:0.00:#NUM!), (CSF.pressure.increased:0:4874:0:11:1.00:#NUM!: 0.00:#NUM!), (Cytomegalovirus.gastrointestinal.ulcer:0:4874:0:11:1.00:#NUM!:0.00:#NUM!), (Dilatation.atrial:0:4874:0:11:1.00:#NUM!:0.00:#NUM!), (Distichiasis:0:4874:0:11:1.00:#NUM!: 0.00:#NUM!), (Dysgraphia:0:4874:0:11:1.00:#NUM!:0.00:#NUM!), (Echocardiogram.abnormal: 0:4874:0:11:1.00:#NUM!:0.00:#NUM!), (Eosinophilic.bronchitis:0:4874:0:11:1.00:#NUM!:0.00: #NUM!), (Faecal.vomiting:0:4874:0:11:1.00:#NUM!:0.00:#NUM!), (Filariasis.lymphatic:0:4874: 0:11:1.00:#NUM!:0.00:#NUM!), (Fusobacterium.test.positive:0:4874:0:11:1.00:#NUM!:0.00:#N UM!), (Gastrinoma.malignant:0:4874:0:11:1.00:#NUM!:0.00:#NUM!), (Genito-pelvic.pain/penetration.disorder:0:4874:0:11:1.00:#NUM!:0.00:#NUM!), (Human.papilloma.viru s.immunisation:0:4874:0:11:1.00:#NUM!:0.00:#NUM!), (Hypopharyngeal.cancer.stage.III:0:487 4:0:11:1.00:#NUM!:0.00:#NUM!), (Intestinal.transit.time:0:4874:0:11:1.00:#NUM!:0.00:#NUM!), (Joint.irrigation:0:4874:0:11:1.00:#NUM!:0.00:#NUM!), (Macroangiopathy:0:4874:0:11:1.00:#NUM!:0.00:#NUM!), (Mastication.disorder:0:4874:0:11:1.00:#NUM!:0.00:#NUM!), (Maxillofaci al.sinus.neoplasm:0:4874:0:11:1.00:#NUM!:0.00:#NUM!), (Medical.observation:0:4874:0:11:1.0 0:#NUM!:0.00:#NUM!), (Melanocytic.naevus:0:4874:0:11:1.00:#NUM!:0.00:#NUM!), (Meningi tis.neonatal:0:4874:0:11:1.00:#NUM!:0.00:#NUM!), (Metatarsalgia:0:4874:0:11:1.00:#NUM!:0. 00:#NUM!), (Myofascitis:0:4874:0:11:1.00:#NUM!:0.00:#NUM!), (Myringitis:0:4874:0:11:1.00: #NUM!:0.00:#NUM!), (Nail.necrosis:0:4874:0:11:1.00:#NUM!:0.00:#NUM!), (Nasal.cyst:0:4874: 0:11:1.00:#NUM!:0.00:#NUM!), (Nasopharyngeal.cancer.recurrent:0:4874:0:11:1.00:#NUM!:0. 00:#NUM!), (Necrotising.enterocolitis.neonatal:0:4874:0:11:1.00:#NUM!:0.00:#NUM!), (Necroti sing.fasciitis.streptococcal:0:4874:0:11:1.00:#NUM!:0.00:#NUM!), (Nipple.pain:0:4874:0:11:1.0 0:#NUM!:0.00:#NUM!), (Oesophagitis.chemical:0:4874:0:11:1.00:#NUM!:0.00:#NUM!), (Onyc halgia:0:4874:0:11:1.00:#NUM!:0.00:#NUM!), (Oral.mucosa.erosion:0:4874:0:11:1.00:#NUM!: 0.00:#NUM!), (Panic.disorder.without.agoraphobia:0:4874:0:11:1.00:#NUM!:0.00:#NUM!), (Par alysis.recurrent.laryngeal.nerve:0:4874:0:11:1.00:#NUM!:0.00:#NUM!), (Persistent.corneal.epith elial.defect:0:4874:0:11:1.00:#NUM!:0.00:#NUM!), (Photorefractive.keratectomy:0:4874:0:11:1. 00:#NUM!:0.00:#NUM!), (Platelet.aggregation.increased:0:4874:0:11:1.00:#NUM!:0.00:#NUM!), (Platelet.dysfunction:0:4874:0:11:1.00:#NUM!:0.00:#NUM!), (Presbyacusis:0:4874:0:11:1.00:#NUM!:0.00:#NUM!), (Prosthetic.vessel.implantation:0:4874:0:11:1.00:#NUM!:0.00:#NUM!), (P ulmonary.oedema.post.fume.inhalation:0:4874:0:11:1.00:#NUM!:0.00:#NUM!), (Purulent.perica rditis:0:4874:0:11:1.00:#NUM!:0.00:#NUM!), (Radial.nerve.palsy:0:4874:0:11:1.00:#NUM!:0.0 0:#NUM!), (Respiratory.syncytial.virus.test:0:4874:0:11:1.00:#NUM!:0.00:#NUM!), (Resting.tre mor:0:4874:0:11:1.00:#NUM!:0.00:#NUM!), (Scan.myocardial.perfusion.abnormal:0:4874:0:11: 1.00:#NUM!:0.00:#NUM!), (Skin.tightness:0:4874:0:11:1.00:#NUM!:0.00:#NUM!), (Spinal.corp ectomy:0:4874:0:11:1.00:#NUM!:0.00:#NUM!), (Stool.analysis:0:4874:0:11:1.00:#NUM!:0.00:#NUM!), (Subdural.effusion:0:4874:0:11:1.00:#NUM!:0.00:#NUM!), (Tanning:0:4874:0:11:1.00:#NUM!:0.00:#NUM!), (Tendon.sheath.incision:0:4874:0:11:1.00:#NUM!:0.00:#NUM!), (Testicula r.hyperfunction:0:4874:0:11:1.00:#NUM!:0.00:#NUM!), (Therapeutic.hypothermia:0:4874:0:11: 1.00:#NUM!:0.00:#NUM!), (Tongue.operation:0:4874:0:11:1.00:#NUM!:0.00:#NUM!), (Tracheo stomy.infection:0:4874:0:11:1.00:#NUM!:0.00:#NUM!), (Uraemic.acidosis:0:4874:0:11:1.00:#N UM!:0.00:#NUM!), (Vertebral.column.mass:0:4874:0:11:1.00:#NUM!:0.00:#NUM!), (Wolff-Parkinson-White.syndrome.congenital:0:4874:0:11:1.00:#NUM!:0.00:#NUM!), (Rhabdoid.tumour:14:4860: 0:11:1.00:1.00:0.56:0.72), (Ganglioneuroblastoma:12:4862:0:11:1.00:1.00:0.52:0.69), (Metastatic. choriocarcinoma:11:4863:0:11:1.00:1.00:0.50:0.67), (Chronic.graft.versus.host.disease.in.liver:10: 4864:0:11:1.00:1.00:0.48:0.65), (Tumour.embolism:10:4864:0:11:1.00:1.00:0.48:0.65), (Immune mediated.necrotising.myopathy:9:4865:0:11:1.00:1.00:0.45:0.62), (Adenoviral.haemorrhagic.cyst itis:8:4866:0:11:1.00:1.00:0.42:0.59), (Autoimmune.colitis:7:4867:0:11:1.00:1.00:0.39:0.56), (Ho dgkin's.disease.lymphocyte.predominance.type.stage.unspecified:7:4867:0:11:1.00:1.00:0.39:0.5 6), (Amblyopia:6:4868:0:11:1.00:1.00:0.35:0.52), (Gastrointestinal.fistula:6:4868:0:11:1.00:1.00: 0.35:0.52), (Multicentric.reticulohistiocytosis:6:4868:0:11:1.00:1.00:0.35:0.52), (Balamuthia.infec tion:5:4869:0:11:1.00:1.00:0.31:0.48), (Infected.fistula:5:4869:0:11:1.00:1.00:0.31:0.48), (Mastiti s.bacterial:5:4869:0:11:1.00:1.00:0.31:0.48), (X-linked.chromosomal.disorder:5:4869:0:11:1.00:1.00:0.31:0.48), (HIV.infection.WHO.clinical.sta ge.I:4:4870:0:11:1.00:1.00:0.27:0.42), (Magnesium.metabolism.disorder:4:4870:0:11:1.00:1.00:0 0.27:0.42), (Open.globe.injury:4:4870:0:11:1.00:1.00:0.27:0.42), (Psychomotor.retardation:4:4870: 0:11:1.00:1.00:0.27:0.42), (Staphylococcal.scalded.skin.syndrome:4:4870:0:11:1.00:1.00:0.27:0. 42), (Abnormal.labour:3:4871:0:11:1.00:1.00:0.21:0.35), (Atopic.keratoconjunctivitis:3:4871:0:11: 1.00:1.00:0.21:0.35), (Atypical.mycobacterial.lymphadenitis:3:4871:1:10:1.00:0.75:0.23:0.35), (Autoimmune.dermatitis:3:4871:0:11:1.00:1.00:0.21:0.35), (Bartholin's.cyst:3:4871:0:11:1.00:1.0 0:0.21:0.35), (Body.height.decreased:3:4871:0:11:1.00:1.00:0.21:0.35), (Coagulation.factor.decre ased:3:4871:0:11:1.00:1.00:0.21:0.35), (Congenital.haematological.disorder:3:4871:0:11:1.00:1.0 0:0.21:0.35), (Dental.examination:3:4871:0:11:1.00:1.00:0.21:0.35), (Extravascular.haemolysis:3: 4871:0:11:1.00:1.00:0.21:0.35), (Joint.range.of.motion.decreased:3:4871:0:11:1.00:1.00:0.21:0.3 5), (Neuroendocrine.breast.tumour:3:4871:0:11:1.00:1.00:0.21:0.35), (Ocular.ischaemic.syndrome: 3:4871:0:11:1.00:1.00:0.21:0.35), (Pineal.parenchymal.neoplasm.malignant:3:4871:0:11:1.00:1. 00:0.21:0.35), (Platelet.function.test.abnormal:3:4871:0:11:1.00:1.00:0.21:0.35), (Post-anoxic.myoclonus:3:4871:0:11:1.00:1.00:0.21:0.35), (Preterm.premature.rupture.of.membranes:3: 4871:0:11:1.00:1.00:0.21:0.35), (Spondylolysis:3:4871:0:11:1.00:1.00:0.21:0.35), (Vascular.graft.complication:3:4871:0:11:1.00:1.00:0.21:0.35), (Adenosquamous.cell.lung.cancer.stage.I:2:4872: 0:11:1.00:1.00:0.15:0.27), (Aicardi's.syndrome:2:4872:0:11:1.00:1.00:0.15:0.27), (Blood.bilirubi n.decreased:2:4872:0:11:1.00:1.00:0.15:0.27), (Cerebellar.ischaemia:2:4872:0:11:1.00:1.00:0.15: 0.27), (Choroid.neoplasm:2:4872:0:11:1.00:1.00:0.15:0.27), (Convulsive.threshold.lowered:2:487 2:0:11:1.00:1.00:0.15:0.27), (Cutaneous.calcification:2:4872:0:11:1.00:1.00:0.15:0.27), (Cytotoxi c.cardiomyopathy:2:4872:0:11:1.00:1.00:0.15:0.27), (Drug.level.therapeutic:2:4872:0:11:1.00:1.0 0:0.15:0.27), (Femoral.artery.occlusion:2:4872:0:11:1.00:1.00:0.15:0.27), (Fracture.treatment:2:4 872:0:11:1.00:1.00:0.15:0.27), (Gastric.mucosal.hypertrophy:2:4872:0:11:1.00:1.00:0.15:0.27), (Gastroenterostomy:2:4872:0:11:1.00:1.00:0.15:0.27), (Haemorrhagic.infarction:2:4872:0:11:1.00:1.00:0.15:0.27), (Hepatic.vascular.thrombosis:2:4872:0:11:1.00:1.00:0.15:0.27), (Hypomenorrho ea:2:4872:0:11:1.00:1.00:0.15:0.27), (Oligohydramnios:2:4872:0:11:1.00:1.00:0.15:0.27), (Perito neal.disorder:2:4872:0:11:1.00:1.00:0.15:0.27), (Pre-existing.condition.improved:2:4872:0:11:1.00:1.00:0.15:0.27), (Ranula:2:4872:0:11:1.00:1.00:0.1 5:0.27), (Renin.increased:2:4872:0:11:1.00:1.00:0.15:0.27), (Retinal.exudates:2:4872:0:11:1.00:1. 00:0.15:0.27), (Skull.fractured.base:2:4872:0:11:1.00:1.00:0.15:0.27), (Stoma.site.inflammation:2:4872:0:11:1.00:1.00:0.15:0.27), (Tumour.treating.fields.therapy:2:4872:0:11:1.00:1.00:0.15:0.27), (Type.I.hypersensitivity:2:4872:0:11:1.00:1.00:0.15:0.27), (Varicose.vein.operation:2:4872:0:1 1:1.00:1.00:0.15:0.27), (Velo-cardio-facial.syndrome:2:4872:0:11:1.00:1.00:0.15:0.27), (Vitamin.B12.increased:2:4872:0:11:1.00:1.00: 0.15:0.27), (Vitreous.degeneration:2:4872:0:11:1.00:1.00:0.15:0.27), (Wound.haematoma:2:4872: 0:11:1.00:1.00:0.15:0.27), (Adrenal.haemorrhage:1:4873:0:11:1.00:1.00:0.08:0.15), (Alcohol.ind uced.persisting.dementia:1:4873:0:11:1.00:1.00:0.08:0.15), (Arteriovenous.graft.site.haemorrhag e:1:4873:0:11:1.00:1.00:0.08:0.15), (Atrial.hypertrophy:1:4873:0:11:1.00:1.00:0.08:0.15), (Atrop hie.blanche:1:4873:0:11:1.00:1.00:0.08:0.15), (Atypical.fracture:1:4873:0:11:1.00:1.00:0.08:0.15), (Blood.bilirubin:1:4873:0:11:1.00:1.00:0.08:0.15), (Blood.thrombin.abnormal:1:4873:0:11:1.00: 1.00:0.08:0.15), (Botulinum.toxin.injection:1:4873:0:11:1.00:1.00:0.08:0.15), (Breast.disorder.fe male:1:4873:0:11:1.00:1.00:0.08:0.15), (Central.nervous.system.fungal.infection:1:4873:0:11:1.0 0:1.00:0.08:0.15), (Chest.crushing:1:4873:0:11:1.00:1.00:0.08:0.15), (Citrobacter.sepsis:1:4873:0: 11:1.00:1.00:0.08:0.15), (Cold.sweat:1:4873:0:11:1.00:1.00:0.08:0.15), (Compulsive.sexual.behav iour:1:4873:0:11:1.00:1.00:0.08:0.15), (Dermatillomania:1:4873:0:11:1.00:1.00:0.08:0.15), (Diet.r efusal:1:4873:0:11:1.00:1.00:0.08:0.15), (Diverticulitis.oesophageal:1:4873:0:11:1.00:1.00:0.08:0 0.15), (Electrocardiogram.ST-T.segment.abnormal:1:4873:0:11:1.00:1.00:0.08:0.15), (Emphysematous.cystitis:1:4873:0:11:1.0 0:1.00:0.08:0.15), (Exposure.to.radiation:1:4873:0:11:1.00:1.00:0.08:0.15), (Factor.XII.deficiency: 1:4873:0:11:1.00:1.00:0.08:0.15), (Fallopian.tube.cancer.stage.III:1:4873:0:11:1.00:1.00:0.08:0. 15), (Fungal.peritonitis:1:4873:0:11:1.00:1.00:0.08:0.15), (Gastrointestinal.anastomotic.leak:1:48 73:0:11:1.00:1.00:0.08:0.15), (Genitourinary.melanoma:1:4873:0:11:1.00:1.00:0.08:0.15), (Gingi val.graft:1:4873:0:11:1.00:1.00:0.08:0.15), (Heat.illness:1:4873:0:11:1.00:1.00:0.08:0.15), (Hepat ic.embolisation:1:4873:0:11:1.00:1.00:0.08:0.15), (Hepatic.enzyme.decreased:1:4873:0:11:1.00:1 0.00:0.08:0.15), (Implant.site.pruritus:1:4873:0:11:1.00:1.00:0.08:0.15), (Incision.site.cellulitis:1:4 873:0:11:1.00:1.00:0.08:0.15), (Inferior.vena.caval.occlusion:1:4873:0:11:1.00:1.00:0.08:0.15), (I odine.uptake.increased:1:4873:0:11:1.00:1.00:0.08:0.15), (Large.intestinal.ulcer.haemorrhage:1:4 873:0:11:1.00:1.00:0.08:0.15), (Malignant.melanoma.of.eyelid:1:4873:0:11:1.00:1.00:0.08:0.15), (Management.of.reproduction:1:4873:0:11:1.00:1.00:0.08:0.15), (Micrographic.skin.surgery:1:48 73:0:11:1.00:1.00:0.08:0.15), (Middle.East.respiratory.syndrome:1:4873:0:11:1.00:1.00:0.08:0.15), (Multiple.chemical.sensitivity:1:4873:0:11:1.00:1.00:0.08:0.15), (Mycobacterium.marinum.infe ction:1:4873:0:11:1.00:1.00:0.08:0.15), (Mycobacterium.tuberculosis.complex.test:1:4873:0:11:1 0.00:1.00:0.08:0.15), (Mycobacterium.ulcerans.infection:1:4873:0:11:1.00:1.00:0.08:0.15), (Narcot ic.bowel.syndrome:1:4873:0:11:1.00:1.00:0.08:0.15), (Neurological.infection:1:4873:0:11:1.00:1. 00:0.08:0.15), (Oophorectomy.bilateral:1:4873:0:11:1.00:1.00:0.08:0.15), (Ovarian.failure.postop erative:1:4873:0:11:1.00:1.00:0.08:0.15), (Paranasal.cyst:1:4873:0:11:1.00:1.00:0.08:0.15), (Penil e.infection:1:4873:0:11:1.00:1.00:0.08:0.15), (pH.urine.decreased:1:4873:0:11:1.00:1.00:0.08:0.1 5), (Post.procedural.pneumonia:1:4873:0:11:1.00:1.00:0.08:0.15), (Progesterone.receptor.assay:1: 4873:0:11:1.00:1.00:0.08:0.15), (Proliferative.injection.therapy:1:4873:0:11:1.00:1.00:0.08:0.15), (Renal.cyst.infection:1:4873:0:11:1.00:1.00:0.08:0.15), (Reverse.tri-iodothyronine.increased:1:4873:0:11:1.00:1.00:0.08:0.15), (Salivary.gland.pain:1:4873:0:11:1.00: 1.00:0.08:0.15), (Smear.cervix:1:4873:0:11:1.00:1.00:0.08:0.15), (Terminal.dribbling:1:4873:0:11: 1.00:1.00:0.08:0.15), (Thymus.abscess:1:4873:0:11:1.00:1.00:0.08:0.15), (Tick-borne.fever:1:4873:0:11:1.00:1.00:0.08:0.15), (Tumour.pseudoprogression:1:4873:0:11:1.00:1.00: 0.08:0.15), (Tympanic.membrane.disorder:1:4873:0:11:1.00:1.00:0.08:0.15), (Vascular.resistance.systemic.decreased:1:4873:0:11:1.00:1.00:0.08:0.15), (Vascular.stent.infection:1:4873:0:11:1.00: 1.00:0.08:0.15), (Vertebroplasty:1:4873:0:11:1.00:1.00:0.08:0.15), (Wound.decomposition:1:487 3:0:11:1.00:1.00:0.08:0.15), (Acute.focal.bacterial.nephritis:0:4873:0:12:1.00:#NUM!:0.00:#NU M!), (Acute.pulmonary.histoplasmosis:0:4873:0:12:1.00:#NUM!:0.00:#NUM!), (Amyotrophy:0:4 873:0:12:1.00:#NUM!:0.00:#NUM!), (Angioscopy:0:4873:0:12:1.00:#NUM!:0.00:#NUM!), (Art eriogram.liver:0:4873:0:12:1.00:#NUM!:0.00:#NUM!), (Benign.soft.tissue.neoplasm:0:4873:0:1 2:1.00:#NUM!:0.00:#NUM!), (Biopsy.colon.abnormal:0:4873:0:12:1.00:#NUM!:0.00:#NUM!), (Blood.fibrinogen:0:4873:0:12:1.00:#NUM!:0.00:#NUM!), (Bone.resorption.test:0:4873:0:12:1.0 0:#NUM!:0.00:#NUM!), (Capillary.permeability.increased:0:4873:0:12:1.00:#NUM!:0.00:#NU M!), (Cardiac.procedure.complication:0:4873:0:12:1.00:#NUM!:0.00:#NUM!), (Carnitine.abnorm al:0:4873:0:12:1.00:#NUM!:0.00:#NUM!), (CD4.lymphocytes.increased:0:4873:0:12:1.00:#NU M!:0.00:#NUM!), (Central.venous.pressure.increased:0:4873:0:12:1.00:#NUM!:0.00:#NUM!), (C hest.wall.tumour:0:4873:0:12:1.00:#NUM!:0.00:#NUM!), (Chronic.eosinophilic.rhinosinusitis:0: 4873:0:12:1.00:#NUM!:0.00:#NUM!), (Citrobacter.test.positive:0:4873:0:12:1.00:#NUM!:0.00:#NUM!), (Congenital.hydronephrosis:0:4873:0:12:1.00:#NUM!:0.00:#NUM!), (Congenital.pancre atic.anomaly:0:4873:0:12:1.00:#NUM!:0.00:#NUM!), (Congenital.spondylolisthesis:0:4873:0:12: 1.00:#NUM!:0.00:#NUM!), (CSF.pressure:0:4873:0:12:1.00:#NUM!:0.00:#NUM!), (Diagnostic. aspiration:0:4873:0:12:1.00:#NUM!:0.00:#NUM!), (Ear.haemorrhage:0:4873:0:12:1.00:#NUM!: 0.00:#NUM!), (Elective.surgery:0:4873:0:12:1.00:#NUM!:0.00:#NUM!), (Electrocardiogram. QT. interval.abnormal:0:4873:0:12:1.00:#NUM!:0.00:#NUM!), (Emotional.poverty:0:4873:0:12:1.00: #NUM!:0.00:#NUM!), (Encephalomalacia:0:4873:0:12:1.00:#NUM!:0.00:#NUM!), (Enterocele:0: 4873:0:12:1.00:#NUM!:0.00:#NUM!), (Excessive.dietary.supplement.intake:0:4873:0:12:1.00:#NUM!:0.00:#NUM!), (Executive.dysfunction:0:4873:0:12:1.00:#NUM!:0.00:#NUM!), (Face.and. mouth.X-ray:0:4873:0:12:1.00:#NUM!:0.00:#NUM!), (Fatty.acid.oxidation.disorder:0:4873:0:12:1.00:#N UM!:0.00:#NUM!), (Foetal.arrhythmia:0:4873:0:12:1.00:#NUM!:0.00:#NUM!), (Follicle.centre.l ymphoma. diffuse.small.cell.lymphoma:0:4873:0:12:1.00:#NUM!:0.00:#NUM!), (Gallbladder.abs cess:0:4873:0:12:1.00:#NUM!:0.00:#NUM!), (Gambling.disorder:0:4873:0:12:1.00:#NUM!:0.00: #NUM!), (Gastric.atony:0:4873:0:12:1.00:#NUM!:0.00:#NUM!), (Gastrointestinal.scan:0:4873:0: 12:1.00:#NUM!:0.00:#NUM!), (Growing.pains:0:4873:0:12:1.00:#NUM!:0.00:#NUM!), (Haemo rrhagic.disease.of.newborn:0:4873:0:12:1.00:#NUM!:0.00:#NUM!), (HIV.peripheral.neuropathy: 0:4873:0:12:1.00:#NUM!:0.00:#NUM!), (Hyperfibrinolysis:0:4873:0:12:1.00:#NUM!:0.00:#NU M!), (Hyperoxia:0:4873:0:12:1.00:#NUM!:0.00:#NUM!), (IIIrd.nerve.paralysis:0:4873:0:12:1.00: #NUM!:0.00:#NUM!), (Laboratory.test:0:4873:0:12:1.00:#NUM!:0.00:#NUM!), (Large.intestine. benign.neoplasm:0:4873:0:12:1.00:#NUM!:0.00:#NUM!), (Leukaemia.cutis:0:4873:0:12:1.00:#NUM!:0.00:#NUM!), (Lung.cyst:0:4873:0:12:1.00:#NUM!:0.00:#NUM!), (Magnetic.resonance.c holangiopancreatography:0:4873:0:12:1.00:#NUM!:0.00:#NUM!), (Mastocytoma:0:4873:0:12:1. 00:#NUM!:0.00:#NUM!), (Medial.tibial.stress.syndrome:0:4873:0:12:1.00:#NUM!:0.00:#NUM!), (Medical.cannabis.therapy:0:4873:0:12:1.00:#NUM!:0.00:#NUM!), (Meningitis.enterococcal:0: 4873:0:12:1.00:#NUM!:0.00:#NUM!), (Muscle.release:0:4873:0:12:1.00:#NUM!:0.00:#NUM!), (Natural.killer.cell.count.increased:0:4873:0:12:1.00:#NUM!:0.00:#NUM!), (Obstetrical.procedur e:0:4873:0:12:1.00:#NUM!:0.00:#NUM!), (Oesophageal.compression:0:4873:0:12:1.00:#NUM!: 0.00:#NUM!), (Oral.allergy.syndrome:0:4873:0:12:1.00:#NUM!:0.00:#NUM!), (Oromandibular. d ystonia:0:4873:0:12:1.00:#NUM!:0.00:#NUM!), (Ovarian.operation:0:4873:0:12:1.00:#NUM!:0. 00:#NUM!), (Paedophilia:0:4873:0:12:1.00:#NUM!:0.00:#NUM!), (Palpable.purpura:0:4873:0:12: 1.00:#NUM!:0.00:#NUM!), (Pancreatogenous.diabetes:0:4873:0:12:1.00:#NUM!:0.00:#NUM!), (Plasma.expander.transfusion:0:4873:0:12:1.00:#NUM!:0.00:#NUM!), (Pneumonia.influenzal:0: 4873:0:12:1.00:#NUM!:0.00:#NUM!), (Poland's.syndrome:0:4873:0:12:1.00:#NUM!:0.00:#NU M!), (Poor.dental.condition:0:4873:0:12:1.00:#NUM!:0.00:#NUM!), (Precancerous.mucosal.lesio n:0:4873:0:12:1.00:#NUM!:0.00:#NUM!), (Primary.hyperthyroidism:0:4873:0:12:1.00:#NUM!:0 0.00:#NUM!), (Product.contamination:0:4873:0:12:1.00:#NUM!:0.00:#NUM!), (Ptosis.repair:0:48 73:0:12:1.00:#NUM!:0.00:#NUM!), (Rhinitis.atrophic:0:4873:0:12:1.00:#NUM!:0.00:#NUM!), (Scan.thyroid.gland:0:4873:0:12:1.00:#NUM!:0.00:#NUM!), (Scrotal.erythema:0:4873:0:12:1.00: #NUM!:0.00:#NUM!), (Stoma.site.pain:0:4873:0:12:1.00:#NUM!:0.00:#NUM!), (Thunderclap.he adache:0:4873:0:12:1.00:#NUM!:0.00:#NUM!), (Tongue.haemorrhage:0:4873:0:12:1.00:#NUM!: 0.00:#NUM!), (Type.IIb.hyperlipidaemia:0:4873:0:12:1.00:#NUM!:0.00:#NUM!), (Ultrafiltratio n.failure:0:4873:0:12:1.00:#NUM!:0.00:#NUM!), (Vaginal.cancer.metastatic:0:4873:0:12:1.00:#NUM!:0.00:#NUM!), (Venogram:0:4873:0:12:1.00:#NUM!:0.00:#NUM!), (Venous.recanalisatio n:0:4873:0:12:1.00:#NUM!:0.00:#NUM!), (Choroid.plexus.carcinoma:18:4855:0:12:1.00:1.00:0. 60:0.75), (Lymphadenopathy.mediastinal:18:4855:0:12:1.00:1.00:0.60:0.75), (Hepatosplenic.T-cell.lymphoma:13:4860:0:12:1.00:1.00:0.52:0.68), (Cortical.dysplasia:11:4862:1:11:1.00:0.92:0. 50:0.65), (Donor.specific.antibody.present:9:4864:0:12:1.00:1.00:0.43:0.60), (Peripheral.T-cell.lymphoma.unspecified.stage.IV:9:4864:0:12:1.00:1.00:0.43:0.60), (T-cell.depletion:8:4865:0:12:1.00:1.00:0.40:0.57), (Tuberculosis.of.genitourinary.system:7:4866:0: 12:1.00:1.00:0.37:0.54), (Extramammary.Paget's.disease:6:4867:0:12:1.00:1.00:0.33:0.50), (Hum an.immunodeficiency.virus.transmission:6:4867:0:12:1.00:1.00:0.33:0.50), (Malignant.neoplasm. of.eye:6:4867:0:12:1.00:1.00:0.33:0.50), (Skin.cancer.metastatic:6:4867:0:12:1.00:1.00:0.33:0.50), (Uterine.hypertonus:6:4867:0:12:1.00:1.00:0.33:0.50), (Adult.T-cell.lymphoma/leukaemia.stage.IV:5:4868:0:12:1.00:1.00:0.29:0.45), (Bacillary.angiomatosis:5:4 868:0:12:1.00:1.00:0.29:0.45), (Human.herpesvirus.8.infection:5:4868:0:12:1.00:1.00:0.29:0.45), (Neurological.decompensation:5:4868:0:12:1.00:1.00:0.29:0.45), (Peripheral.arthritis:5:4868:0:1 2:1.00:1.00:0.29:0.45), (Uterine.cervix.dilation.procedure:5:4868:0:12:1.00:1.00:0.29:0.45), (Acut e.undifferentiated.leukaemia:4:4869:0:12:1.00:1.00:0.25:0.40), (Echinococciasis:4:4869:0:12:1.0 0:1.00:0.25:0.40), (Ileal.stenosis:4:4869:0:12:1.00:1.00:0.25:0.40), (Leukocyte.antigen.B-27.positive:4:4869:0:12:1.00:1.00:0.25:0.40), (Neisseria.infection:4:4869:0:12:1.00:1.00:0.25:0.4 0), (Scimitar.syndrome:4:4869:0:12:1.00:1.00:0.25:0.40), (ACTH-producing.pituitary.tumour:3:4870:0:12:1.00:1.00:0.20:0.33), (Diabetic.end.stage.renal.disease:3: 4870:0:12:1.00:1.00:0.20:0.33), (Habitual.abortion:3:4870:0:12:1.00:1.00:0.20:0.33), (Hypocompl ementaemia:3:4870:0:12:1.00:1.00:0.20:0.33), (Mandibulofacial.dysostosis:3:4870:0:12:1.00:1.0 0:0.20:0.33), (Placental.disorder:3:4870:0:12:1.00:1.00:0.20:0.33), (Renal.transplant.failure:3:487 0:0:12:1.00:1.00:0.20:0.33), (Rhesus.incompatibility:3:4870:0:12:1.00:1.00:0.20:0.33), (Testicular.germ.cell.tumour.mixed:3:4870:0:12:1.00:1.00:0.20:0.33), (Adrenal.adenoma:2:4871:0:12:1.00: 1.00:0.14:0.25), (American.trypanosomiasis:2:4871:0:12:1.00:1.00:0.14:0.25), (Amnestic.disorder: 2:4871:0:12:1.00:1.00:0.14:0.25), (Blast.cell.proliferation:2:4871:0:12:1.00:1.00:0.14:0.25), (Bod y.mass.index.decreased:2:4871:0:12:1.00:1.00:0.14:0.25), (Bone.giant.cell.tumour.malignant:2:4 871:0:12:1.00:1.00:0.14:0.25), (Cheilosis:2:4871:0:12:1.00:1.00:0.14:0.25), (Corona.virus.infectio n:2:4871:0:12:1.00:1.00:0.14:0.25), (Encephalitis.protozoal:2:4871:0:12:1.00:1.00:0.14:0.25), (Eo sinophilic.colitis:2:4871:0:12:1.00:1.00:0.14:0.25), (Flavivirus.infection:2:4871:0:12:1.00:1.00:0. 14:0.25), (Foetal.growth.abnormality:2:4871:0:12:1.00:1.00:0.14:0.25), (Fundoscopy.normal:2:48 71:0:12:1.00:1.00:0.14:0.25), (Hyperleukocytosis:2:4871:0:12:1.00:1.00:0.14:0.25), (Mastocytic.l eukaemia:2:4871:0:12:1.00:1.00:0.14:0.25), (Meningioma.benign:2:4871:0:12:1.00:1.00:0.14:0.2 5), (Metastases.to.neck:2:4871:0:12:1.00:1.00:0.14:0.25), (Penile.squamous.cell.carcinoma:2:487 1:0:12:1.00:1.00:0.14:0.25), (Pituitary.tumour.removal:2:4871:0:12:1.00:1.00:0.14:0.25), (Primar y.mediastinal.large.B-cell.lymphoma.stage.III:2:4871:0:12:1.00:1.00:0.14:0.25), (Pulmonary.valve.disease:2:4871:0:12:1.00:1.00:0.14:0.25), (Respiratory.rate.increased:2:4871:0:12:1.00:1.00:0.14:0.25), (SUNCT.syn drome:2:4871:0:12:1.00:1.00:0.14:0.25), (Urinary.bladder.rupture:2:4871:0:12:1.00:1.00:0.14:0.2 5), (Uterine.enlargement:2:4871:0:12:1.00:1.00:0.14:0.25), (Wound.necrosis:2:4871:0:12:1.00:1.0 0:0.14:0.25), (Adrenal.blockade.therapy:1:4872:0:12:1.00:1.00:0.08:0.14), (Amylase:1:4872:0:12: 1.00:1.00:0.08:0.14), (Blast.cells.present:1:4872:0:12:1.00:1.00:0.08:0.14), (Cardiac.failure.high.o utput:1:4872:0:12:1.00:1.00:0.08:0.14), (Chronic.right.ventricular.failure:1:4872:0:12:1.00:1.00:0 0.08:0.14), (Corneal.perforation:1:4872:0:12:1.00:1.00:0.08:0.14), (Cortisol.abnormal:1:4872:0:12: 1.00:1.00:0.08:0.14), (Cystopexy:1:4872:0:12:1.00:1.00:0.08:0.14), (Drug.effect.prolonged:1:487 2:0:12:1.00:1.00:0.08:0.14), (Ear.operation:1:4872:0:12:1.00:1.00:0.08:0.14), (Epididymal.cyst:1: 4872:0:12:1.00:1.00:0.08:0.14), (Facet.joint.block:1:4872:0:12:1.00:1.00:0.08:0.14), (Fusobacteri um.infection:1:4872:0:12:1.00:1.00:0.08:0.14), (Glucose-6-phosphate.dehydrogenase.deficiency:1:4872:0:12:1.00:1.00:0.08:0.14), (Haemophilia.B.with.anti. factor.IX:1:4872:0:12:1.00:1.00:0.08:0.14), (Heart.valve.calcification:1:4872:0:12:1.00:1.00:0.08: 0.14), (Hostility:1:4872:0:12:1.00:1.00:0.08:0.14), (Incision.site.complication:1:4872:0:12:1.00:1 0.00:0.08:0.14), (Large.intestinal.polypectomy:1:4872:0:12:1.00:1.00:0.08:0.14), (Lipase:1:4872:0: 12:1.00:1.00:0.08:0.14), (Lissencephaly:1:4872:0:12:1.00:1.00:0.08:0.14), (Long.QT.syndrome.co ngenital:1:4872:0:12:1.00:1.00:0.08:0.14), (Mean.cell.volume.increased:1:4872:0:12:1.00:1.00:0. 08:0.14), (Mevalonate.kinase.deficiency:1:4872:0:12:1.00:1.00:0.08:0.14), (Mismatched.donor.bo ne.marrow.transplantation.therapy:1:4872:0:12:1.00:1.00:0.08:0.14), (N-ras.gene.mutation:1:4872:0:12:1.00:1.00:0.08:0.14), (Obstetric.infection:1:4872:0:12:1.00:1.00:0. 08:0.14), (Optic.disc.drusen:1:4872:0:12:1.00:1.00:0.08:0.14), (Paroxysmal.sympathetic.hyperacti vity:1:4872:0:12:1.00:1.00:0.08:0.14), (Pericarditis.infective:1:4872:0:12:1.00:1.00:0.08:0.14), (P ostoperative.hypopituitarism:1:4872:0:12:1.00:1.00:0.08:0.14), (PUVA:1:4872:0:12:1.00:1.00:0. 08:0.14), (Smooth.muscle.cell.neoplasm:1:4872:0:12:1.00:1.00:0.08:0.14), (Stab.wound:1:4872:0: 12:1.00:1.00:0.08:0.14), (Subdural.hygroma:1:4872:0:12:1.00:1.00:0.08:0.14), (Thalassaemia.alp ha:1:4872:0:12:1.00:1.00:0.08:0.14), (Tonsillitis.streptococcal:1:4872:0:12:1.00:1.00:0.08:0.14), (Tyrosine.kinase.mutation:1:4872:0:12:1.00:1.00:0.08:0.14), (Urine.analysis.normal:1:4872:0:12: 1.00:1.00:0.08:0.14), (Vestibulitis:1:4872:0:12:1.00:1.00:0.08:0.14), (Acanthamoeba.keratitis:0:4 872:0:13:1.00:#NUM!:0.00:#NUM!), (Alpha.interferon.therapy:0:4872:0:13:1.00:#NUM!:0.00:#NUM!), (Alveolar.lung.disease:0:4872:0:13:1.00:#NUM!:0.00:#NUM!), (Anal.candidiasis:0:4872: 0:13:1.00:#NUM!:0.00:#NUM!), (Antiprolactin.therapy:0:4872:0:13:1.00:#NUM!:0.00:#NUM!), (Atypical.mycobacterial.pneumonia:0:4872:0:13:1.00:#NUM!:0.00:#NUM!), (Basosquamous.ca rcinoma:0:4872:0:13:1.00:#NUM!:0.00:#NUM!), (Biopsy.blood.vessel.abnormal:0:4872:0:13:1.0 0:#NUM!:0.00:#NUM!), (Blood.osmolarity:0:4872:0:13:1.00:#NUM!:0.00:#NUM!), (Bronchial.h aemorrhage:0:4872:0:13:1.00:#NUM!:0.00:#NUM!), (Burn.dressing:0:4872:0:13:1.00:#NUM!:0. 00:#NUM!), (Carbon.dioxide.increased:0:4872:0:13:1.00:#NUM!:0.00:#NUM!), (Cataract.subcap sular:0:4872:0:13:1.00:#NUM!:0.00:#NUM!), (Cholelithotomy:0:4872:0:13:1.00:#NUM!:0.00:#NUM!), (Culture.urine:0:4872:0:13:1.00:#NUM!:0.00:#NUM!), (Dental.plaque:0:4872:0:13:1.00: #NUM!:0.00:#NUM!), (Dopamine.dysregulation.syndrome:0:4872:0:13:1.00:#NUM!:0.00:#NU M!), (Drug. administered.at.inappropriate.site:0:4872:0:13:1.00:#NUM!:0.00:#NUM!), (Electrocar diogram.ambulatory:0:4872:0:13:1.00:#NUM!:0.00:#NUM!), (Eyelid.pain:0:4872:0:13:1.00:#N UM!:0.00:#NUM!), (Female.sex.hormone.level:0:4872:0:13:1.00:#NUM!:0.00:#NUM!), (Femora 1.nerve.injury:0:4872:0:13:1.00:#NUM!:0.00:#NUM!), (Foetal.therapeutic.procedure:0:4872:0:13: 1.00:#NUM!:0.00:#NUM!), (Fungal.test:0:4872:0:13:1.00:#NUM!:0.00:#NUM!), (Gallbladder.c ancer.stage.III:0:4872:0:13:1.00:#NUM!:0.00:#NUM!), (Genital.infection.viral:0:4872:0:13:1.00: #NUM!:0.00:#NUM!), (Glucose.tolerance.test:0:4872:0:13:1.00:#NUM!:0.00:#NUM!), (Hepatitis.chronic.persistent:0:4872:0:13:1.00:#NUM!:0.00:#NUM!), (Hepatosplenomegaly:0:4872:0:13:1. 00:#NUM!:0.00:#NUM!), (Hereditary.pancreatitis:0:4872:0:13:1.00:#NUM!:0.00:#NUM!), (Hyp ertrophic.scar:0:4872:0:13:1.00:#NUM!:0.00:#NUM!), (Ichthyosis.acquired:0:4872:0:13:1.00:#N UM!:0.00:#NUM!), (Inability.to.afford.medication:0:4872:0:13:1.00:#NUM!:0.00:#NUM!), (Intr a-cerebral.aneurysm.operation:0:4872:0:13:1.00:#NUM!:0.00:#NUM!), (Lipoprotein. (a).abnormal: 0:4872:0:13:1.00:#NUM!:0.00:#NUM!), (Livedo.reticularis:0:4872:0:13:1.00:#NUM!:0.00:#NU M!), (Mean.arterial.pressure.increased:0:4872:0:13:1.00:#NUM!:0.00:#NUM!), (Medical.device.c hange:0:4872:0:13:1.00:#NUM!:0.00:#NUM!), (Meningorrhagia:0:4872:0:13:1.00:#NUM!:0.00: #NUM!), (Mucocutaneous.candidiasis:0:4872:0:13:1.00:#NUM!:0.00:#NUM!), (Neurological.reh abilitation:0:4872:0:13:1.00:#NUM!:0.00:#NUM!), (Obsessive-compulsive.symptom:0:4872:0:13:1.00:#NUM!:0.00:#NUM!), (Oculomucocutaneous.syndrome: 0:4872:0:13:1.00:#NUM!:0.00:#NUM!), (Oesophageal.ulcer.haemorrhage:0:4872:0:13:1.00:#NU M!:0.00:#NUM!), (Oxygen.consumption:0:4872:0:13:1.00:#NUM!:0.00:#NUM!), (Pancreatic.sar coma:0:4872:0:13:1.00:#NUM!:0.00:#NUM!), (Pericarditis.malignant:0:4872:0:13:1.00:#NUM!: 0.00:#NUM!), (Perseveration:0:4872:0:13:1.00:#NUM!:0.00:#NUM!), (pH.body.fluid.increased:0: 4872:0:13:1.00:#NUM!:0.00:#NUM!), (Positive.vessel.remodelling:0:4872:0:13:1.00:#NUM!:0. 00:#NUM!), (Post.angioplasty.restenosis:0:4872:0:13:1.00:#NUM!:0.00:#NUM!), (Post.procedur al.discharge:0:4872:0:13:1.00:#NUM!:0.00:#NUM!), (Prescription.drug.used.without.a.prescripti on:0:4872:0:13:1.00:#NUM!:0.00:#NUM!), (Product.reconstitution.quality.issue:0:4872:0:13:1.0 0:#NUM!:0.00:#NUM!), (Prostate.induration:0:4872:0:13:1.00:#NUM!:0.00:#NUM!), (Protein.to tal.increased:0:4872:0:13:1.00:#NUM!:0.00:#NUM!), (Pseudomonas.aeruginosa.meningitis:0:48 72:0:13:1.00:#NUM!:0.00:#NUM!), (Pseudovasculitis:0:4872:0:13:1.00:#NUM!:0.00:#NUM!), (Psychogenic.pain.disorder:0:4872:0:13:1.00:#NUM!:0.00:#NUM!), (Red.blood.cell.count.normal: 0:4872:0:13:1.00:#NUM!:0.00:#NUM!), (Renal.haemorrhage:0:4872:0:13:1.00:#NUM!:0.00:#N UM!), (Rickettsialpox:0:4872:0:13:1.00:#NUM!:0.00:#NUM!), (Salt.craving:0:4872:0:13:1.00:#NUM!:0.00:#NUM!), (Serratia.sepsis:0:4872:0:13:1.00:#NUM!:0.00:#NUM!), (Sleep-related.eating.disorder:0:4872:0:13:1.00:#NUM!:0.00:#NUM!), (Solvent.sensitivity:0:4872:0:13: 1.00:#NUM!:0.00:#NUM!), (Spinal.cord.operation:0:4872:0:13:1.00:#NUM!:0.00:#NUM!), (Spu turn. purulent:0:4872:0:13:1.00:#NUM!:0.00:#NUM!), (Strabismus:0:4872:0:13:1.00:#NUM!:0.0 0:#NUM!), (Tertiary.syphilis:0:4872:0:13:1.00:#NUM!:0.00:#NUM!), (Thromboembolectomy:0: 4872:0:13:1.00:#NUM!:0.00:#NUM!), (Tobacco.poisoning:0:4872:0:13:1.00:#NUM!:0.00:#NU M!), (Tooth.erosion:0:4872:0:13:1.00:#NUM!:0.00:#NUM!), (Total.cholesterol/HDL.ratio.abnor mal:0:4872:0:13:1.00:#NUM!:0.00:#NUM!), (Traumatic.intracranial.haematoma:0:4872:0:13:1.0 0:#NUM!:0.00:#NUM!), (Tri-iodothyronine:0:4872:0:13:1.00:#NUM!:0.00:#NUM!), (Vaginal.operation:0:4872:0:13:1.00:#N UM!:0.00:#NUM!), (Waist.circumference.increased:0:4872:0:13:1.00:#NUM!:0.00:#NUM!), (Ho dgkin's.disease.lymphocyte.depletion.type.stage.unspecified:12:4860:0:13:1.00:1.00:0.48:0.65), (Gait.inability:11:4861:0:13:1.00:1.00:0.46:0.63), (Non-Hodgkin's.lymphoma.stage.II:11:4861:0:13:1.00:1.00:0.46:0.63), (Extragonadal.primary.germ.cel 1.tumour:10:4862:0:13:1.00:1.00:0.43:0.61), (Neuroectodermal.neoplasm:10:4862:0:13:1.00:1.00:0.43:0.61), (Small.cell.lung.cancer.limited.stage:10:4862:0:13:1.00:1.00:0.43:0.61), (Follicle.cent re.lymphoma,.follicular.grade.I,.II,.III.stage.III:9:4863:0:13:1.00:1.00:0.41:0.58), (Gastrointestina 1.viral.infection:9:4863:0:13:1.00:1.00:0.41:0.58), (Composite.lymphoma:8:4864:0:13:1.00:1.00: 0.38:0.55), (Yolk.sac.tumour.site.unspecified:8:4864:0:13:1.00:1.00:0.38:0.55), (Colorectal.cance r.stage.III:7:4865:0:13:1.00:1.00:0.35:0.52), (Granulocytosis:7:4865:0:13:1.00:1.00:0.35:0.52), (A esthesioneuroblastoma:6:4866:0:13:1.00:1.00:0.32:0.48), (Necrobiosis.lipoidica.diabeticorum:6:4 866:0:13:1.00:1.00:0.32:0.48), (Cervical.dysplasia:5:4867:0:13:1.00:1.00:0.28:0.43), (Chronic.ly mphocytic.leukaemia.stage.2:5:4867:0:13:1.00:1.00:0.28:0.43), (Drug.clearance.decreased:5:486 7:0:13:1.00:1.00:0.28:0.43), (Hepatitis.C.RNA:5:4867:0:13:1.00:1.00:0.28:0.43), (Nodular.regene rative.hyperplasia:5:4867:0:13:1.00:1.00:0.28:0.43), (Perivascular.dermatitis:5:4867:0:13:1.00:1. 00:0.28:0.43), (Rash.papulosquamous:5:4867:0:13:1.00:1.00:0.28:0.43), (Tumour.invasion:5:486 7:0:13:1.00:1.00:0.28:0.43), (Acquired.epileptic.aphasia:4:4868:0:13:1.00:1.00:0.24:0.38), (Activ ated.protein.C.resistance:4:4868:0:13:1.00:1.00:0.24:0.38), (Allergic.keratitis:4:4868:0:13:1.00:1. 00:0.24:0.38), (Cytomegalovirus.gastroenteritis:4:4868:0:13:1.00:1.00:0.24:0.38), (Dental.fluorid e.therapy:4:4868:0:13:1.00:1.00:0.24:0.38), (Lafora's.myoclonic.epilepsy:4:4868:0:13:1.00:1.00: 0.24:0.38), (Retinitis.histoplasma:4:4868:0:13:1.00:1.00:0.24:0.38), (Retroperitoneal.neoplasm:4: 4868:0:13:1.00:1.00:0.24:0.38), (Uterine.pain:4:4868:0:13:1.00:1.00:0.24:0.38), (Wiskott-Aldrich.syndrome:4:4868:0:13:1.00:1.00:0.24:0.38), (Adenoidal.disorder:3:4869:0:13:1.00:1.00: 0.19:0.32), (Apituitarism:3:4869:0:13:1.00:1.00:0.19:0.32), (Deformity:3:4869:0:13:1.00:1.00:0.1 9:0.32), (Intestinal.infarction:3:4869:0:13:1.00:1.00:0.19:0.32), (Lactobacillus.infection:3:4869:0: 13:1.00:1.00:0.19:0.32), (Neonatal.diabetes.mellitus:3:4869:0:13:1.00:1.00:0.19:0.32), (Retroperit oneal.neoplasm.metastatic:3:4869:0:13:1.00:1.00:0.19:0.32), (Rhesus.antigen.negative:3:4869:0: 13:1.00:1.00:0.19:0.32), (Stroke.volume.decreased:3:4869:0:13:1.00:1.00:0.19:0.32), (Abdominal. migraine:2:4870:0:13:1.00:1.00:0.13:0.24), (Acne.pustular:2:4870:0:13:1.00:1.00:0.13:0.24), (Ad enosquamous.carcinoma.of.the.cervix:2:4870:0:13:1.00:1.00:0.13:0.24), (Anti-glomerular.basement.membrane.antibody:2:4870:0:13:1.00:1.00:0.13:0.24), (Arteriovenous.fistul a.site.complication:2:4870:0:13:1.00:1.00:0.13:0.24), (Blood.ketone.body:2:4870:0:13:1.00:1.00: 0.13:0.24), (Bone.infarction:2:4870:0:13:1.00:1.00:0.13:0.24), (Breathing-related.sleep.disorder:2:4870:0:13:1.00:1.00:0.13:0.24), (Cavemous.sinus.thrombosis:2:4870:0:1 3:1.00:1.00:0.13:0.24), (Congenital.coronary.artery.malformation:2:4870:0:13:1.00:1.00:0.13:0.2 4), (Distributive.shock:2:4870:0:13:1.00:1.00:0.13:0.24), (Drug.effect.variable:2:4870:0:13:1.00:1 0.00:0.13:0.24), (Dysmorphism:2:4870:0:13:1.00:1.00:0.13:0.24), (Fertility.increased:2:4870:0:13: 1.00:1.00:0.13:0.24), (Hepatitis.B.surface.antibody.positive:2:4870:0:13:1.00:1.00:0.13:0.24), (Hi strionic.personality.disorder:2:4870:0:13:1.00:1.00:0.13:0.24), (Joint.resurfacing.surgery:2:4870: 0:13:1.00:1.00:0.13:0.24), (Lupus.myocarditis:2:4870:0:13:1.00:1.00:0.13:0.24), (Non-tobacco.user:2:4870:0:13:1.00:1.00:0.13:0.24), (Pityriasis:2:4870:0:13:1.00:1.00:0.13:0.24), (Plast ic.surgery:2:4870:0:13:1.00:1.00:0.13:0.24), (Pulmonary.vein.stenosis:2:4870:0:13:1.00:1.00:0.1 3:0.24), (Retinal.pigmentation:2:4870:0:13:1.00:1.00:0.13:0.24), (Superinfection.fungal:2:4870:0: 13:1.00:1.00:0.13:0.24), (Ulna.fracture:2:4870:0:13:1.00:1.00:0.13:0.24), (Ulnar.nerve.palsy:2:48 70:0:13:1.00:1.00:0.13:0.24), (Anamnestic.reaction:1:4871:0:13:1.00:1.00:0.07:0.13), (Angelman's.syndrome:1:4871:0:13:1.00:1.00:0.07:0.13), (Avulsion.fracture:1:4871:0:13:1.00:1.00:0.07:0.13), (Basal.ganglia.haemorrhage:1:4871:0:13:1.00:1.00:0.07:0.13), (Bladder.cancer.stage.III:1:4871: 0:13:1.00:1.00:0.07:0.13), (Blood.glucagon.abnormal:1:4871:0:13:1.00:1.00:0.07:0.13), (Blood.so dium:1:4871:0:13:1.00:1.00:0.07:0.13), (Brain.lobectomy:1:4871:0:13:1.00:1.00:0.07:0.13), (Bur kholderia.mallei.infection:1:4871:0:13:1.00:1.00:0.07:0.13), (Cavopulmonary.anastomosis:1:487 1:0:13:1.00:1.00:0.07:0.13), (Cerebrospinal.fistula:1:4871:0:13:1.00:1.00:0.07:0.13), (Conductive. deafness:1:4871:0:13:1.00:1.00:0.07:0.13), (Cow's.milk.intolerance:1:4871:0:13:1.00:1.00:0.07:0 0.13), (Cranial.operation:1:4871:0:13:1.00:1.00:0.07:0.13), (Delirium.febrile:1:4871:0:13:1.00:1.00: 0.07:0.13), (Ear.neoplasm:1:4871:0:13:1.00:1.00:0.07:0.13), (Enterovesical.fistula:1:4871:0:13:1. 00:1.00:0.07:0.13), (Enzyme.activity.abnormal:1:4871:0:13:1.00:1.00:0.07:0.13), (Epileptic.aura: 1:4871:0:13:1.00:1.00:0.07:0.13), (Epileptic.psychosis:1:4871:0:13:1.00:1.00:0.07:0.13), (Erythe ma.migrans:1:4871:0:13:1.00:1.00:0.07:0.13), (Euthanasia:1:4871:0:13:1.00:1.00:0.07:0.13), (Gas trointestinal.necrosis:1:4871:0:13:1.00:1.00:0.07:0.13), (Glioneuronal.tumour:1:4871:0:13:1.00:1 0.00:0.07:0.13), (Gonadotrophin.deficiency:1:4871:0:13:1.00:1.00:0.07:0.13), (Haemorrhoid.infecti on:1:4871:0:13:1.00:1.00:0.07:0.13), (Hallucination,.tactile:1:4871:0:13:1.00:1.00:0.07:0.13), (HI V.infection.CDC.Group.III:1:4871:0:13:1.00:1.00:0.07:0.13), (Hyperchromic.anaemia:1:4871:0: 13:1.00:1.00:0.07:0.13), (Induration:1:4871:0:13:1.00:1.00:0.07:0.13), (Intraocular.pressure.fluctu ation:1:4871:0:13:1.00:1.00:0.07:0.13), (Lumbosacral.plexus.injury:1:4871:0:13:1.00:1.00:0.07:0 0.13), (Lung.adenocarcinoma.stage.I:1:4871:0:13:1.00:1.00:0.07:0.13), (Mild.mental.retardation:1: 4871:0:13:1.00:1.00:0.07:0.13), (Nail.bed.infection.fungal:1:4871:0:13:1.00:1.00:0.07:0.13), (Nail.injury:1:4871:0:13:1.00:1.00:0.07:0.13), (Otoplasty:1:4871:0:13:1.00:1.00:0.07:0.13), (Ovarian.h yperfunction:1:4871:0:13:1.00:1.00:0.07:0.13), (Partner.stress:1:4871:0:13:1.00:1.00:0.07:0.13), (Phaeochromocytoma.malignant:1:4871:0:13:1.00:1.00:0.07:0.13), (Phobia.of.flying:1:4871:0:13: 1.00:1.00:0.07:0.13), (Pituitary.cyst:1:4871:0:13:1.00:1.00:0.07:0.13), (Postictal.state:1:4871:0:13: 1.00:1.00:0.07:0.13), (Postoperative.delirium:1:4871:0:13:1.00:1.00:0.07:0.13), (Precursor.B-lymphoblastic.lymphoma.stage.IV:1:4871:0:13:1.00:1.00:0.07:0.13), (Pyelonephritis.chronic:1:4 871:0:13:1.00:1.00:0.07:0.13), (Reaction.to.azo-dyes:1:4871:0:13:1.00:1.00:0.07:0.13), (Residual.urine.volume:1:4871:0:13:1.00:1.00:0.07:0.13), (Sensory.level.abnormal:1:4871:0:13:1.00:1.00:0.07:0.13), (Serum.ferritin:1:4871:0:13:1.00:1.00: 0.07:0.13), (Soft.tissue.haemorrhage:1:4871:0:13:1.00:1.00:0.07:0.13), (Sperm.concentration.dec reased:1:4871:0:13:1.00:1.00:0.07:0.13), (Systemic.viral.infection:1:4871:0:13:1.00:1.00:0.07:0.1 3), (Thrombectomy:1:4871:0:13:1.00:1.00:0.07:0.13), (Tumour.haemorrhage:1:4871:0:13:1.00:1. 00:0.07:0.13), (Turbinectomy:1:4871:0:13:1.00:1.00:0.07:0.13), (White.matter.lesion:1:4871:0:13: 1.00:1.00:0.07:0.13), (Wrong.technique.in.drug.usage.process:1:4871:0:13:1.00:1.00:0.07:0.13), (X-ray.with.contrast.upper.gastrointestinal.tract:1:4871:0:13:1.00:1.00:0.07:0.13), (Abnormal.sleep-related.event:0:4871:0:14:1.00:#NUM!:0.00:#NUM!), (Acute.painful.neuropathy.of.rapid.glycae mic.control:0:4871:0:14:1.00:#NUM!:0.00:#NUM!), (Agitation.postoperative:0:4871:0:14:1.00:#NUM!:0.00:#NUM!), (Alcoholic.relative:0:4871:0:14:1.00:#NUM!:0.00:#NUM!), (Anal.erosion: 0:4871:0:14:1.00:#NUM!:0.00:#NUM!), (Application.site.rash:0:4871:0:14:1.00:#NUM!:0.00:#NUM!), (Aspiration.tracheal:0:4871:0:14:1.00:#NUM!:0.00:#NUM!), (Biliary.dilatation:0:4871:0: 14:1.00:#NUM!:0.00:#NUM!), (Bladder.neoplasm.surgery:0:4871:0:14:1.00:#NUM!:0.00:#NU M!), (Blood.corticosterone:0:4871:0:14:1.00:#NUM!:0.00:#NUM!), (Blood.zinc.abnormal:0:4871: 0:14:1.00:#NUM!:0.00:#NUM!), (Cardiac.output:0:4871:0:14:1.00:#NUM!:0.00:#NUM!), (Carti lage.atrophy:0:4871:0:14:1.00:#NUM!:0.00:#NUM!), (Circumcision:0:4871:0:14:1.00:#NUM!:0. 00:#NUM!), (Conjunctival.melanoma:0:4871:0:14:1.00:#NUM!:0.00:#NUM!), (Diabetic.dyslipid aemia:0:4871:0:14:1.00:#NUM!:0.00:#NUM!), (Double.outlet.right.ventricle:0:4871:0:14:1.00:#NUM!:0.00:#NUM!), (Eagle.Barrett.syndrome:0:4871:0:14:1.00:#NUM!:0.00:#NUM!), (Econom ic.problem:0:4871:0:14:1.00:#NUM!:0.00:#NUM!), (Endotracheal.intubation.complication:0:487 1:0:14:1.00:#NUM!:0.00:#NUM!), (Erythropenia:0:4871:0:14:1.00:#NUM!:0.00:#NUM!), (Femo ral.nerve.palsy:0:4871:0:14:1.00:#NUM!:0.00:#NUM!), (Fine.motor.skill.dysfunction:0:4871:0:1 4:1.00:#NUM!:0.00:#NUM!), (Forced.expiratory.volume.decreased:0:4871:0:14:1.00:#NUM!:0. 00:#NUM!), (Gingival.atrophy:0:4871:0:14:1.00:#NUM!:0.00:#NUM!), (Hepatitis.non-A.non-B:0:4871:0:14:1.00:#NUM!:0.00:#NUM!), (Histamine.intolerance:0:4871:0:14:1.00:#NUM!:0.0 0:#NUM!), (Hungry.bone.syndrome:0:4871:0:14:1.00:#NUM!:0.00:#NUM!), (Hyperviscosity.syn drome:0:4871:0:14:1.00:#NUM!:0.00:#NUM!), (Influenza.virus.test.positive:0:4871:0:14:1.00:#NUM!:0.00:#NUM!), (Intra-thoracic.aortic.aneurysm.repair:0:4871:0:14:1.00:#NUM!:0.00:#NUM!), (Joint.hyperextension:0: 4871:0:14:1.00:#NUM!:0.00:#NUM!), (Kussmaul.respiration:0:4871:0:14:1.00:#NUM!:0.00:#N UM!), (Lactose.tolerance.test:0:4871:0:14:1.00:#NUM!:0.00:#NUM!), (Left.atrial.hypertrophy:0: 4871:0:14:1.00:#NUM!:0.00:#NUM!), (Left.ventricular.end-diastolic.pressure.increased:0:4871:0:14:1.00:#NUM!:0.00:#NUM!), (Oestradiol:0:4871:0:14:1.0 0:#NUM!:0.00:#NUM!), (Orgasmic.sensation.decreased:0:4871:0:14:1.00:#NUM!:0.00:#NUM!), (Pancreatic.fistula:0:4871:0:14:1.00:#NUM!:0.00:#NUM!), (Parathyroid.tumour.benign:0:4871: 0:14:1.00:#NUM!:0.00:#NUM!), (Penicillium.infection:0:4871:0:14:1.00:#NUM!:0.00:#NUM!), (Peptostreptococcus.infection:0:4871:0:14:1.00:#NUM!:0.00:#NUM!), (Phlebitis.infective:0:487 1:0:14:1.00:#NUM!:0.00:#NUM!), (Respiratory.tract.irritation:0:4871:0:14:1.00:#NUM!:0.00:#N UM!), (Retroperitoneal.abscess:0:4871:0:14:1.00:#NUM!:0.00:#NUM!), (Ruptured.cerebral.aneur ysm:0:4871:0:14:1.00:#NUM!:0.00:#NUM!), (Scar.pain:0:4871:0:14:1.00:#NUM!:0.00:#NUM!), (Splenic.lesion:0:4871:0:14:1.00:#NUM!:0.00:#NUM!), (Tri-iodothyronine.increased:0:4871:0:14:1.00:#NUM!:0.00:#NUM!), (Urinary.anastomotic.leak:0:48 71:0:14:1.00:#NUM!:0.00:#NUM!), (White.blood.cell.scan:0:4871:0:14:1.00:#NUM!:0.00:#NU M!), (Whole.body.scan:0:4871:0:14:1.00:#NUM!:0.00:#NUM!), (Wound.drainage:0:4871:0:14:1. 00:#NUM!:0.00:#NUM!), (Wound.healing.normal:0:4871:0:14:1.00:#NUM!:0.00:#NUM!), (Hod gkin's.disease.stage.III:13:4858:0:14:1.00:1.00:0.48:0.65), (Antiangiogenic.therapy:8:4863:0:14: 1.00:1.00:0.36:0.53), (Intravascular.papillary.endothelial.hyperplasia:8:4863:0:14:1.00:1.00:0.36: 0.53), (Convalescent:7:4864:0:14:1.00:1.00:0.33:0.50), (Foetal.death:7:4864:0:14:1.00:1.00:0.33: 0.50), (Paraneoplastic.dermatomyositis:7:4864:0:14:1.00:1.00:0.33:0.50), (Pulmonary.arterial.pres sure.increased:7:4864:0:14:1.00:1.00:0.33:0.50), (Hepatopulmonary.syndrome:6:4865:0:14:1.00: 1.00:0.30:0.46), (Leukaemia.recurrent.prophylaxis:6:4865:0:14:1.00:1.00:0.30:0.46), (Meconium. aspiration.syndrome:5:4866:0:14:1.00:1.00:0.26:0.42), (Menopausal.symptoms.prophylaxis:5:48 66:0:14:1.00:1.00:0.26:0.42), (Psychomotor.skills.impaired:5:4866:0:14:1.00:1.00:0.26:0.42), (W est.Nile.virus.test.positive:5:4866:0:14:1.00:1.00:0.26:0.42), (Abortion.threatened:4:4867:0:14:1. 00:1.00:0.22:0.36), (Anti-glomerular.basement.membrane.antibody.positive:4:4867:0:14:1.00:1.00:0.22:0.36), (Chronic.gra ft.versus.host.disease.in.intestine:4:4867:0:14:1.00:1.00:0.22:0.36), (Fibrinous.bronchitis:4:4867: 0:14:1.00:1.00:0.22:0.36), (Granular.cell.tumour:4:4867:0:14:1.00:1.00:0.22:0.36), (Intrinsic.facto r.deficiency:4:4867:0:14:1.00:1.00:0.22:0.36), (Mature.B-cell.type.acute.leukaemia:4:4867:0:14:1.00:1.00:0.22:0.36), (Miller.Fisher.syndrome:4:4867:0:14:1.00:1.00:0.22:0.36), (Mucinous.breast.carcinoma:4:4867:0:14:1.00:1.00:0.22:0.36), (Mycosis.fu ngoides.refractory:4:4867:0:14:1.00:1.00:0.22:0.36), (Neuromyotonia:4:4867:0:14:1.00:1.00:0.22: 0.36), (Pancreatic.leak:4:4867:0:14:1.00:1.00:0.22:0.36), (Pyomyositis:4:4867:0:14:1.00:1.00:0.2 2:0.36), (Respiratory.tract.neoplasm:4:4867:0:14:1.00:1.00:0.22:0.36), (Testicular.yolk.sac.tumou r:4:4867:0:14:1.00:1.00:0.22:0.36), (Xanthogranuloma:4:4867:0:14:1.00:1.00:0.22:0.36), (Acute.e ncephalitis.with.refractory,.repetitive.partial.seizures:3:4868:0:14:1.00:1.00:0.18:0.30), (Antidepr essant.drug.level.below.therapeutic:3:4868:0:14:1.00:1.00:0.18:0.30), (Biotherapy:3:4868:0:14:1. 00:1.00:0.18:0.30), (Glucagonoma:3:4868:0:14:1.00:1.00:0.18:0.30), (Ketosis-prone.diabetes.mellitus:3:4868:0:14:1.00:1.00:0.18:0.30), (Meningoradiculitis:3:4868:0:14:1.00:1 0.00:0.18:0.30), (Myocardial.strain:3:4868:0:14:1.00:1.00:0.18:0.30), (Neurodevelopmental.disord er:3:4868:0:14:1.00:1.00:0.18:0.30), (Omenn.syndrome:3:4868:0:14:1.00:1.00:0.18:0.30), (Perica rdial.excision:3:4868:0:14:1.00:1.00:0.18:0.30), (Polyhydramnios:3:4868:0:14:1.00:1.00:0.18:0.3 0), (Prostate.cancer.stage.II:3:4868:0:14:1.00:1.00:0.18:0.30), (Secretory.adenoma.of.pituitary:3:4 868:0:14:1.00:1.00:0.18:0.30), (Subretinal.fluid:3:4868:0:14:1.00:1.00:0.18:0.30), (Univentricular.heart:3:4868:0:14:1.00:1.00:0.18:0.30), (Anaplastic.meningioma:2:4869:0:14:1.00:1.00:0.13:0.2 2), (Anti-neuronal.antibody.positive:2:4869:0:14:1.00:1.00:0.13:0.22), (Avian.influenza:2:4869:0:14:1.00: 1.00:0.13:0.22), (Blood.creatinine.abnormal:2:4869:0:14:1.00:1.00:0.13:0.22), (CHA2DS2-VASc.annual.stroke.risk.high:2:4869:0:14:1.00:1.00:0.13:0.22), (Chronotropic.incompetence:2:4 869:0:14:1.00:1.00:0.13:0.22), (Cytokine.abnormal:2:4869:0:14:1.00:1.00:0.13:0.22), (Dry.gangre ne:2:4869:0:14:1.00:1.00:0.13:0.22), (Erythrasma:2:4869:0:14:1.00:1.00:0.13:0.22), (Herpes.simp lex.meningoencephalitis:2:4869:0:14:1.00:1.00:0.13:0.22), (Leukostasis.syndrome:2:4869:0:14:1. 00:1.00:0.13:0.22), (Lip.squamous.cell.carcinoma:2:4869:0:14:1.00:1.00:0.13:0.22), (Malformatio n.venous:2:4869:0:14:1.00:1.00:0.13:0.22), (Malignant.renal.hypertension:2:4869:0:14:1.00:1.00: 0.13:0.22), (Neutrophil.toxic.granulation.present:2:4869:0:14:1.00:1.00:0.13:0.22), (Pituitary.gon adotropin.hyperfunction:2:4869:0:14:1.00:1.00:0.13:0.22), (Pulmonary.arteriopathy:2:4869:0:14: 1.00:1.00:0.13:0.22), (Urethral.dilatation:2:4869:0:14:1.00:1.00:0.13:0.22), (Urinary.tract.infectio n.viral:2:4869:0:14:1.00:1.00:0.13:0.22), (Vitritis:2:4869:0:14:1.00:1.00:0.13:0.22), (Anterior.cha mber.inflammation:1:4870:0:14:1.00:1.00:0.07:0.13), (Aortic.aneurysm.rupture:1:4870:0:14:1.00: 1.00:0.07:0.13), (Arachnoid.cyst:1:4870:0:14:1.00:1.00:0.07:0.13), (Arterial.fibrosis:1:4870:0:14: 1.00:1.00:0.07:0.13), (Blood.donor:1:4870:0:14:1.00:1.00:0.07:0.13), (Blood.glucose.normal:1:48 70:0:14:1.00:1.00:0.07:0.13), (Blood.product.transfusion.dependent:1:4870:0:14:1.00:1.00:0.07:0 0.13), (Caplan's.syndrome:1:4870:0:14:1.00:1.00:0.07:0.13), (Coagulation.factor.VIII.level.increas ed:1:4870:0:14:1.00:1.00:0.07:0.13), (Cochlea.implant:1:4870:0:14:1.00:1.00:0.07:0.13), (Cyst.ru pture:1:4870:0:14:1.00:1.00:0.07:0.13), (Eczema.impetiginous:1:4870:0:14:1.00:1.00:0.07:0.13), (Eczema.infantile:1:4870:0:14:1.00:1.00:0.07:0.13), (Electromyogram:1:4870:0:14:1.00:1.00:0.07: 0.13), (Encephalitis.fungal:1:4870:0:14:1.00:1.00:0.07:0.13), (Food.intolerance:1:4870:0:14:1.00: 1.00:0.07:0.13), (Giant.papillary.conjunctivitis:1:4870:0:14:1.00:1.00:0.07:0.13), (Hepatitis.A.im munisation:1:4870:0:14:1.00:1.00:0.07:0.13), (Hyperproteinaemia:1:4870:0:14:1.00:1.00:0.07:0. 13), (Impetigo.herpetiformis:1:4870:0:14:1.00:1.00:0.07:0.13), (Laryngeal.cancer.stage.IV:1:4870: 0:14:1.00:1.00:0.07:0.13), (Liposarcoma.recurrent:1:4870:0:14:1.00:1.00:0.07:0.13), (Micturition.frequency.decreased:1:4870:0:14:1.00:1.00:0.07:0.13), (Middle.ear.disorder:1:4870:0:14:1.00:1. 00:0.07:0.13), (Myelodysplastic.syndrome.unclassifiable:1:4870:0:14:1.00:1.00:0.07:0.13), (Obstr uctive.defaecation:1:4870:0:14:1.00:1.00:0.07:0.13), (Ophthalmic.vein.thrombosis:1:4870:0:14:1. 00:1.00:0.07:0.13), (Osteoprotegerin.ligand:1:4870:0:14:1.00:1.00:0.07:0.13), (Pancreas.divisum: 1:4870:0:14:1.00:1.00:0.07:0.13), (Pericardial.neoplasm:1:4870:0:14:1.00:1.00:0.07:0.13), (Physi cal.assault:1:4870:0:14:1.00:1.00:0.07:0.13), (Prothrombin.time.shortened:1:4870:0:14:1.00:1.00: 0.07:0.13), (Renal.vein.compression:1:4870:0:14:1.00:1.00:0.07:0.13), (Rocky.mountain.spotted.f ever:1:4870:0:14:1.00:1.00:0.07:0.13), (Sexual.abuse:1:4870:0:14:1.00:1.00:0.07:0.13), (Sudden. visual.loss:1:4870:0:14:1.00:1.00:0.07:0.13), (Sympathomimetic.effect:1:4870:0:14:1.00:1.00:0.0 7:0.13), (Type.III.hyperlipidaemia:1:4870:0:14:1.00:1.00:0.07:0.13), (Vascular.catheterisation:1:4 870:0:14:1.00:1.00:0.07:0.13), (Abscess.of.salivary.gland:0:4870:0:15:1.00:#NUM!:0.00:#NUM!), (Adrenal.cyst:0:4870:0:15:1.00:#NUM!:0.00:#NUM!), (Arteritis.coronary:0:4870:0:15:1.00:#N UM!:0.00:#NUM!), (B-cell.unclassifiable.lymphoma.low.grade:0:4870:0:15:1.00:#NUM!:0.00:#NUM!), (Balanitis.candi da:0:4870:0:15:1.00:#NUM!:0.00:#NUM!), (Basilar.artery.occlusion:0:4870:0:15:1.00:#NUM!:0 0.00:#NUM!), (Blood.erythropoietin:0:4870:0:15:1.00:#NUM!:0.00:#NUM!), (Bone.marrow.myel ogram.abnormal:0:4870:0:15:1.00:#NUM!:0.00:#NUM!), (Catheter.site.inflammation:0:4870:0:1 5:1.00:#NUM!:0.00:#NUM!), (Cerebral.salt-wasting.syndrome:0:4870:0:15:1.00:#NUM!:0.00:#NUM!), (Clostridial.sepsis:0:4870:0:15:1.00: #NUM!:0.00:#NUM!), (Conjunctival.neoplasm:0:4870:0:15:1.00:#NUM!:0.00:#NUM!), (Corneal.leukoma:0:4870:0:15:1.00:#NUM!:0.00:#NUM!), (Coronary.artery.restenosis:0:4870:0:15:1.00: #NUM!:0.00:#NUM!), (Cytology.abnormal:0:4870:0:15:1.00:#NUM!:0.00:#NUM!), (Cytomegal ovirus.pancreatitis:0:4870:0:15:1.00:#NUM!:0.00:#NUM!), (Delayed.recovery.from.anaesthesia: 0:4870:0:15:1.00:#NUM!:0.00:#NUM!), (Dialysis.disequilibrium.syndrome:0:4870:0:15:1.00:#N UM!:0.00:#NUM!), (Dieulafoy's.vascular.malformation:0:4870:0:15:1.00:#NUM!:0.00:#NUM!), (Ejaculation.failure:0:4870:0:15:1.00:#NUM!:0.00:#NUM!), (Eosinophilic.leukaemia:0:4870:0:1 5:1.00:#NUM!:0.00:#NUM!), (Exercise.adequate:0:4870:0:15:1.00:#NUM!:0.00:#NUM!), (Fluid. intake.restriction:0:4870:0:15:1.00:#NUM!:0.00:#NUM!), (Free.fatty.acids:0:4870:0:15:1.00:#N UM!:0.00:#NUM!), (Genital.abscess:0:4870:0:15:1.00:#NUM!:0.00:#NUM!), (Gonorrhoea:0:487 0:0:15:1.00:#NUM!:0.00:#NUM!), (Haemodynamic.rebound:0:4870:0:15:1.00:#NUM!:0.00:#N UM!), (Holt-Oram.syndrome:0:4870:0:15:1.00:#NUM!:0.00:#NUM!), (Implant.site.pain:0:4870:0:15:1.00:#N UM!:0.00:#NUM!), (Intraventricular.haemorrhage:0:4870:0:15:1.00:#NUM!:0.00:#NUM!), (Men tal.fatigue:0:4870:0:15:1.00:#NUM!:0.00:#NUM!), (Mutism:0:4870:0:15:1.00:#NUM!:0.00:#NU M!), (Oesophageal.atresia:0:4870:0:15:1.00:#NUM!:0.00:#NUM!), (Pulmonary.valve.replacemen t:0:4870:0:15:1.00:#NUM!:0.00:#NUM!), (Pyopneumothorax:0:4870:0:15:1.00:#NUM!:0.00:#N UM!), (Renal.cyst.haemorrhage:0:4870:0:15:1.00:#NUM!:0.00:#NUM!), (Sacral.radiculopathy:0: 4870:0:15:1.00:#NUM!:0.00:#NUM!), (Skin.operation:0:4870:0:15:1.00:#NUM!:0.00:#NUM!), (Somatotropin.stimulation.test:0:4870:0:15:1.00:#NUM!:0.00:#NUM!), (Staphylococcal.mediasti nitis:0:4870:0:15:1.00:#NUM!:0.00:#NUM!), (Transfusion-related.circulatory.overload:0:4870:0:15:1.00:#NUM!:0.00:#NUM!), (Urine.odour.abnormal:0:48 70:0:15:1.00:#NUM!:0.00:#NUM!), (Vaginal.pessary.insertion:0:4870:0:15:1.00:#NUM!:0.00:#NUM!), (Vascular.cognitive.impairment:0:4870:0:15:1.00:#NUM!:0.00:#NUM!), (Vascular.stent. occlusion:0:4870:0:15:1.00:#NUM!:0.00:#NUM!), (Ventriculo-peritoneal.shunt:0:4870:0:15:1.00:#NUM!:0.00:#NUM!), (Familial.haemophagocytic.lymphohist iocytosis:12:4858:0:15:1.00:1.00:0.44:0.62), (Acute.interstitial.pneumonitis:11:4859:0:15:1.00:1. 00:0.42:0.59), (Myasthenia.gravis.crisis:8:4862:0:15:1.00:1.00:0.35:0.52), (Neuroborreliosis:8:48 62:0:15:1.00:1.00:0.35:0.52), (Ovarian.epithelial.cancer.metastatic:8:4862:0:15:1.00:1.00:0.35:0. 52), (Plasmodium.vivax.infection:8:4862:0:15:1.00:1.00:0.35:0.52), (Lymphoid.tissue.hyperplasia: 7:4863:0:15:1.00:1.00:0.32:0.48), (Mantle.cell.lymphoma.stage.II:7:4863:0:15:1.00:1.00:0.32:0. 48), (Breast.angiosarcoma:6:4864:0:15:1.00:1.00:0.29:0.44), (Cryptococcus.test.positive:6:4864:0: 15:1.00:1.00:0.29:0.44), (Vena.cava.embolism:6:4864:0:15:1.00:1.00:0.29:0.44), (Bone.operation: 5:4865:0:15:1.00:1.00:0.25:0.40), (Chemotherapy.multiple.agents.systemic:5:4865:0:15:1.00:1.0 0:0.25:0.40), (Complement.factor:5:4865:0:15:1.00:1.00:0.25:0.40), (Costal.cartilage.fracture:5:4 865:0:15:1.00:1.00:0.25:0.40), (Glomerulonephritis.acute:5:4865:0:15:1.00:1.00:0.25:0.40), (Stre ptococcus.test:5:4865:0:15:1.00:1.00:0.25:0.40), (Foetal.cardiac.disorder:4:4866:0:15:1.00:1.00:0 0.21:0.35), (Gender.dysphoria:4:4866:0:15:1.00:1.00:0.21:0.35), (Haemorrhage.in.pregnancy:4:486 6:0:15:1.00:1.00:0.21:0.35), (Infectious.mononucleosis:4:4866:0:15:1.00:1.00:0.21:0.35), (Oral.m ucosal.eruption:4:4866:0:15:1.00:1.00:0.21:0.35), (Phaehyphomycosis:4:4866:0:15:1.00:1.00:0.2 1:0.35), (Soft.tissue.swelling:4:4866:0:15:1.00:1.00:0.21:0.35), (Tumour.thrombosis:4:4866:0:15: 1.00:1.00:0.21:0.35), (Ureaplasma.infection:4:4866:0:15:1.00:1.00:0.21:0.35), (Vascular.graft.occ lusion:4:4866:0:15:1.00:1.00:0.21:0.35), (Abdominal.wall.haematoma:3:4867:0:15:1.00:1.00:0.1 7:0.29), (Acute.leukaemia.in.remission:3:4867:0:15:1.00:1.00:0.17:0.29), (Adhesion:3:4867:0:15: 1.00:1.00:0.17:0.29), (Benign.ovarian.tumour:3:4867:0:15:1.00:1.00:0.17:0.29), (Carditis:3:4867: 0:15:1.00:1.00:0.17:0.29), (Cholestatic.pruritus:3:4867:0:15:1.00:1.00:0.17:0.29), (Diaphragmatic.spasm:3:4867:0:15:1.00:1.00:0.17:0.29), (Hereditary.leiomyomatosis.renal.cell.carcinoma:3:486 7:0:15:1.00:1.00:0.17:0.29), (Mammoplasty:3:4867:0:15:1.00:1.00:0.17:0.29), (Neuronal.ceroid.li pofuscinosis:3:4867:0:15:1.00:1.00:0.17:0.29), (Pleocytosis:3:4867:0:15:1.00:1.00:0.17:0.29), (Pn eumonitis.chemical:3:4867:0:15:1.00:1.00:0.17:0.29), (Prosthesis.user:3:4867:0:15:1.00:1.00:0.1 7:0.29), (Ureteroscopy:3:4867:0:15:1.00:1.00:0.17:0.29), (Autoimmune.nephritis:2:4868:0:15:1.0 0:1.00:0.12:0.21), (Bladder.adenocarcinoma.stage.unspecified:2:4868:0:15:1.00:1.00:0.12:0.21), (Blood.parathyroid.hormone:2:4868:0:15:1.00:1.00:0.12:0.21), (Cerebral.fungal.infection:2:4868: 0:15:1.00:1.00:0.12:0.21), (Coagulation.test:2:4868:0:15:1.00:1.00:0.12:0.21), (Congenital.dysker atosis:2:4868:0:15:1.00:1.00:0.12:0.21), (Ear.neoplasm.malignant:2:4868:0:15:1.00:1.00:0.12:0.2 1), (Endocrine.neoplasm:2:4868:0:15:1.00:1.00:0.12:0.21), (Euthyroid.sick.syndrome:2:4868:0:15:1.00:1.00:0.12:0.21), (Fibromuscular.dysplasia:2:4868:0:15:1.00:1.00:0.12:0.21), (Investigation.a bnormal:2:4868:0:15:1.00:1.00:0.12:0.21), (Malignant.nipple.neoplasm.male:2:4868:0:15:1.00:1. 00:0.12:0.21), (Metal.poisoning:2:4868:0:15:1.00:1.00:0.12:0.21), (Myocarditis.infectious:2:4868: 0:15:1.00:1.00:0.12:0.21), (Pharyngeal.abscess:2:4868:0:15:1.00:1.00:0.12:0.21), (Prophylaxis.of.vaginal.flora.imbalance:2:4868:0:15:1.00:1.00:0.12:0.21), (Pulmonary.necrosis:2:4868:0:15:1.00: 1.00:0.12:0.21), (Sense.of.oppression:2:4868:0:15:1.00:1.00:0.12:0.21), (Synovial.sarcoma.recurr ent:2:4868:0:15:1.00:1.00:0.12:0.21), (Thyroiditis.subacute:2:4868:0:15:1.00:1.00:0.12:0.21), (Tr eatment.noncompliance:2:4868:0:15:1.00:1.00:0.12:0.21), (Urinary.bladder.toxicity:2:4868:0:15: 1.00:1.00:0.12:0.21), (Venous.haemorrhage:2:4868:0:15:1.00:1.00:0.12:0.21), (Withdrawal.bleed: 2:4868:0:15:1.00:1.00:0.12:0.21), (Acinic.cell.carcinoma.of.salivary.gland:1:4869:0:15:1.00:1.00: 0.06:0.12), (Acquired.lipoatrophic.diabetes:1:4869:0:15:1.00:1.00:0.06:0.12), (Angiogram.pulmo nary:1:4869:0:15:1.00:1.00:0.06:0.12), (Bartter's.syndrome:1:4869:0:15:1.00:1.00:0.06:0.12), (Bi opsy.vulva:1:4869:0:15:1.00:1.00:0.06:0.12), (Bladder.neck.operation:1:4869:0:15:1.00:1.00:0.0 6:0.12), (Bronchial.oedema:1:4869:0:15:1.00:1.00:0.06:0.12), (Bum.infection:1:4869:0:15:1.00:1. 00:0.06:0.12), (Cerebral.haematoma:1:4869:0:15:1.00:1.00:0.06:0.12), (Delusional.perception:1:4 869:0:15:1.00:1.00:0.06:0.12), (Depersonalisation:1:4869:0:15:1.00:1.00:0.06:0.12), (Distractibili ty:1:4869:0:15:1.00:1.00:0.06:0.12), (Drug.dispensed.to.wrong.patient:1:4869:0:15:1.00:1.00:0.0 6:0.12), (Extraocular.muscle.disorder:1:4869:0:15:1.00:1.00:0.06:0.12), (Head.and.neck.cancer.st age.IV:1:4869:0:15:1.00:1.00:0.06:0.12), (Hydrops.foetalis:1:4869:0:15:1.00:1.00:0.06:0.12), (Hy pobarism:1:4869:0:15:1.00:1.00:0.06:0.12), (Irregular.breathing:1:4869:0:15:1.00:1.00:0.06:0.12), (Kounis.syndrome:1:4869:0:15:1.00:1.00:0.06:0.12), (Marrow.hyperplasia:1:4869:0:15:1.00:1.0 0:0.06:0.12), (Microlithiasis:1:4869:0:15:1.00:1.00:0.06:0.12), (Nasopharyngeal.cancer.metastatic: 1:4869:0:15:1.00:1.00:0.06:0.12), (Neuralgic.amyotrophy:1:4869:0:15:1.00:1.00:0.06:0.12), (Neu rogenic.shock:1:4869:0:15:1.00:1.00:0.06:0.12), (Ocular.neoplasm:1:4869:0:15:1.00:1.00:0.06:0. 12), (Oesophageal.squamous.cell.carcinoma.stage.II:1:4869:0:15:1.00:1.00:0.06:0.12), (Phlebitis. deep:1:4869:0:15:1.00:1.00:0.06:0.12), (Radiation.proctitis:1:4869:0:15:1.00:1.00:0.06:0.12), (Sc hizotypal.personality.disorder:1:4869:0:15:1.00:1.00:0.06:0.12), (Spine.malformation:1:4869:0:1 5:1.00:1.00:0.06:0.12), (Ulnar.tunnel.syndrome:1:4869:0:15:1.00:1.00:0.06:0.12), (Upper.respirat ory.tract.infection.bacterial:1:4869:0:15:1.00:1.00:0.06:0.12), (Ureteric.dilatation:1:4869:0:15:1.0 0:1.00:0.06:0.12), (Venous.ulcer.pain:1:4869:0:15:1.00:1.00:0.06:0.12), (Viral.uveitis:1:4869:0:1 5:1.00:1.00:0.06:0.12), (Acoustic.neuroma:0:4869:0:16:1.00:#NUM!:0.00:#NUM!), (Afterbirth.p ain:0:4869:0:16:1.00:#NUM!:0.00:#NUM!), (Albuminuria:0:4869:0:16:1.00:#NUM!:0.00:#NU M!), (Antasthmatic.drug.level.therapeutic:0:4869:0:16:1.00:#NUM!:0.00:#NUM!), (Anticachectic.therapy:0:4869:0:16:1.00:#NUM!:0.00:#NUM!), (B-cell.small.lymphocytic.lymphoma.refractory:0:4869:0:16:1.00:#NUM!:0.00:#NUM!), (Becker's.n aevus:0:4869:0:16:1.00:#NUM!:0.00:#NUM!), (Blood.lactate.dehydrogenase.abnormal:0:4869:0: 16:1.00:#NUM!:0.00:#NUM!), (Borderline.mental.impairment:0:4869:0:16:1.00:#NUM!:0.00:#NUM!), (Bradykinesia:0:4869:0:16:1.00:#NUM!:0.00:#NUM!), (Capillaritis:0:4869:0:16:1.00:#N UM!:0.00:#NUM!), (Choanal.atresia:0:4869:0:16:1.00:#NUM!:0.00:#NUM!), (Chondrosarcoma. metastatic:0:4869:0:16:1.00:#NUM!:0.00:#NUM!), (Chromaturia:0:4869:0:16:1.00:#NUM!:0.00: #NUM!), (Counterfeit.drug.administered:0:4869:0:16:1.00:#NUM!:0.00:#NUM!), (Cycloplegia:0: 4869:0:16:1.00:#NUM!:0.00:#NUM!), (Cystitis.klebsiella:0:4869:0:16:1.00:#NUM!:0.00:#NU M!), (Ear.tube.insertion:0:4869:0:16:1.00:#NUM!:0.00:#NUM!), (Enterobacter.bacteraemia:0:486 9:0:16:1.00:#NUM!:0.00:#NUM!), (Eosinophilic.pustular.folliculitis:0:4869:0:16:1.00:#NUM!:0. 00:#NUM!), (Extranodal.marginal.zone.B-cell.lymphoma.
(MALT.type).recurrent:0:4869:0:16:1.00:#NUM!:0.00:#NUM!), (Eyelid.operatio n:0:4869:0:16:1.00:#NUM!:0.00:#NUM!), (Female.sterilisation:0:4869:0:16:1.00:#NUM!:0.00:#NUM!), (Gastrointestinal.vascular.malformation:0:4869:0:16:1.00:#NUM!:0.00:#NUM!), (Glauc omatocyclitic.crises:0:4869:0:16:1.00:#NUM!:0.00:#NUM!), (Haemangioma.of.skin:0:4869:0:16: 1.00:#NUM!:0.00:#NUM!), (Hepatic.cyst:0:4869:0:16:1.00:#NUM!:0.00:#NUM!), (Hepatic.enz yme:0:4869:0:16:1.00:#NUM!:0.00:#NUM!), (Hypoaesthesia.eye:0:4869:0:16:1.00:#NUM!:0.00: #NUM!), (Inbom.error.in.primary.bile.acid.synthesis:0:4869:0:16:1.00:#NUM!:0.00:#NUM!), (I nfected.seroma:0:4869:0:16:1.00:#NUM!:0.00:#NUM!), (Infective.aortitis:0:4869:0:16:1.00:#N UM!:0.00:#NUM!), (Infective.keratitis:0:4869:0:16:1.00:#NUM!:0.00:#NUM!), (Joint.contractur e:0:4869:0:16:1.00:#NUM!:0.00:#NUM!), (Legionella.test.positive:0:4869:0:16:1.00:#NUM!:0.0 0:#NUM!), (Meningitis.candida:0:4869:0:16:1.00:#NUM!:0.00:#NUM!), (Myocardial.fibrosis:0:4 869:0:16:1.00:#NUM!:0.00:#NUM!), (Nasal.mucosal.disorder:0:4869:0:16:1.00:#NUM!:0.00:#N UM!), (Nephrocalcinosis:0:4869:0:16:1.00:#NUM!:0.00:#NUM!), (Otitis.externa.bacterial:0:4869: 0:16:1.00:#NUM!:0.00:#NUM!), (Otosalpingitis:0:4869:0:16:1.00:#NUM!:0.00:#NUM!), (Pepti c.ulcer.helicobacter:0:4869:0:16:1.00:#NUM!:0.00:#NUM!), (Perineurial.cyst:0:4869:0:16:1.00:#NUM!:0.00:#NUM!), (Peristalsis.visible:0:4869:0:16:1.00:#NUM!:0.00:#NUM!), (Pleural.infecti on.bacterial:0:4869:0:16:1.00:#NUM!:0.00:#NUM!), (Poor.sanitation:0:4869:0:16:1.00:#NUM!: 0.00:#NUM!), (Post.procedural.stroke:0:4869:0:16:1.00:#NUM!:0.00:#NUM!), (Prostate.cryoabla tion:0:4869:0:16:1.00:#NUM!:0.00:#NUM!), (Pulmonary.eosinophilia:0:4869:0:16:1.00:#NUM!: 0.00:#NUM!), (Radiation.neuropathy:0:4869:0:16:1.00:#NUM!:0.00:#NUM!), (Sepsis.syndrome: 0:4869:0:16:1.00:#NUM!:0.00:#NUM!), (Serum.serotonin.increased:0:4869:0:16:1.00:#NUM!:0. 00:#NUM!), (Serum.sickness-like.reaction:0:4869:0:16:1.00:#NUM!:0.00:#NUM!), (Skeletal.survey:0:4869:0:16:1.00:#NUM!: 0.00:#NUM!), (Stemotomy:0:4869:0:16:1.00:#NUM!:0.00:#NUM!), (Subdural.empyema:0:4869: 0:16:1.00:#NUM!:0.00:#NUM!), (Sympatholysis:0:4869:0:16:1.00:#NUM!:0.00:#NUM!), (Thera peutic.emesis:0:4869:0:16:1.00:#NUM!:0.00:#NUM!), (Therapy.non-responder:0:4869:0:16:1.00:#NUM!:0.00:#NUM!), (Tricuspid.valve.prolapse:0:4869:0:16:1.00:#NUM!:0.00:#NUM!), (Vascular.access.site.infection:0:4869:0:16:1.00:#NUM!:0.00:#NUM!), (Vi sceral.congestion:0:4869:0:16:1.00:#NUM!:0.00:#NUM!), (Vulval.neoplasm:0:4869:0:16:1.00:#NUM!:0.00:#NUM!), (Transplant.dysfunction:12:4857:0:16:1.00:1.00:0.43:0.60), (Epilepsy.cong enital:11:4858:0:16:1.00:1.00:0.41:0.58), (Liver.and.small.intestine.transplant:9:4860:0:16:1.00:1 0.00:0.36:0.53), (T-cell.lymphoma.refractory:9:4860:0:16:1.00:1.00:0.36:0.53), (Primary.adrenal.insufficiency:8:486 1:0:16:1.00:1.00:0.33:0.50), (Cell-mediated.cytotoxicity:7:4862:0:16:1.00:1.00:0.30:0.47), (Rhabdomyosarcoma.recurrent:7:4862:0: 16:1.00:1.00:0.30:0.47), (Second.primary.malignancy:7:4862:0:16:1.00:1.00:0.30:0.47), (Acute.c utaneous.lupus.erythematosus:6:4863:0:16:1.00:1.00:0.27:0.43), (Dactylitis:6:4863:0:16:1.00:1.0 0:0.27:0.43), (Hepatosplenic.candidiasis:6:4863:0:16:1.00:1.00:0.27:0.43), (Lactation.inhibition.th erapy:6:4863:0:16:1.00:1.00:0.27:0.43), (Malignant.hydatidiform.mole:6:4863:0:16:1.00:1.00:0.2 7:0.43), (Blood.heavy.metal.increased:5:4864:0:16:1.00:1.00:0.24:0.38), (Cerebellar.syndrome:5: 4864:0:16:1.00:1.00:0.24:0.38), (Lymphohistiocytosis:5:4864:0:16:1.00:1.00:0.24:0.38), (Periphe ral.T-cell.lymphoma.unspecified.refractory:5:4864:0:16:1.00:1.00:0.24:0.38), (Alveolar.soft.part.sarco ma.metastatic:4:4865:0:16:1.00:1.00:0.20:0.33), (Bone.giant.cell.tumour.benign:4:4865:0:16:1.00: 1.00:0.20:0.33), (Cartilage.operation:4:4865:0:16:1.00:1.00:0.20:0.33), (Eye.abscess:4:4865:0:16: 1.00:1.00:0.20:0.33), (Hypertelorism.of.orbit:4:4865:0:16:1.00:1.00:0.20:0.33), (Metastases.to.ch est.wall:4:4865:0:16:1.00:1.00:0.20:0.33), (Radiation.necrosis:4:4865:0:16:1.00:1.00:0.20:0.33), (Renal.hypoplasia:4:4865:0:16:1.00:1.00:0.20:0.33), (Substance.abuser:4:4865:0:16:1.00:1.00:0.2 0:0.33), (Tongue.cancer.recurrent:4:4865:0:16:1.00:1.00:0.20:0.33), (Aspiration.joint:3:4866:0:16: 1.00:1.00:0.16:0.27), (Blood.HIV.RNA:3:4866:0:16:1.00:1.00:0.16:0.27), (Endothelial.dysfuncti on:3:4866:0:16:1.00:1.00:0.16:0.27), (Gender.identity.disorder:3:4866:0:16:1.00:1.00:0.16:0.27), (Gorham's.disease:3:4866:0:16:1.00:1.00:0.16:0.27), (Hepatitis.B.immunisation:3:4866:0:16:1.00: 1.00:0.16:0.27), (Herpes.zoster.meningoencephalitis:3:4866:0:16:1.00:1.00:0.16:0.27), (Immuno globulins:3:4866:0:16:1.00:1.00:0.16:0.27), (Inflammatory.carcinoma.of.breast.stage.III:3:4866:0: 16:1.00:1.00:0.16:0.27), (Peroneal.nerve.injury:3:4866:0:16:1.00:1.00:0.16:0.27), (PFAPA.syndr ome:3:4866:0:16:1.00:1.00:0.16:0.27), (Pseudallescheria.infection:3:4866:0:16:1.00:1.00:0.16:0. 27), (Pyruvate.kinase.deficiency.anaemia:3:4866:0:16:1.00:1.00:0.16:0.27), (Abscess.bacterial:2:4 867:0:16:1.00:1.00:0.11:0.20), (Bladder.transitional.cell.carcinoma.stage.I:2:4867:0:16:1.00:1.00: 0.11:0.20), (Blood.immunoglobulin.M.abnormal:2:4867:0:16:1.00:1.00:0.11:0.20), (Carcinoid.hea rt.disease:2:4867:0:16:1.00:1.00:0.11:0.20), (Cheilitis.granulomatosa:2:4867:0:16:1.00:1.00:0.11: 0.20), (Diffuse.idiopathic.skeletal.hyperostosis:2:4867:0:16:1.00:1.00:0.11:0.20), (Exercise.tolera nce.decreased:2:4867:0:16:1.00:1.00:0.11:0.20), (Foetal.alcohol.syndrome:2:4867:0:16:1.00:1.00:0.11:0.20), (Hepatic.artery.thrombosis:2:4867:0:16:1.00:1.00:0.11:0.20), (Idiosyncratic.drug.reac tion:2:4867:0:16:1.00:1.00:0.11:0.20), (Incisional.hernia.repair:2:4867:0:16:1.00:1.00:0.11:0.20), (Iron.binding.capacity.total.abnormal:2:4867:0:16:1.00:1.00:0.11:0.20), (Joint.surgery:2:4867:0:1 6:1.00:1.00:0.11:0.20), (Malignant.atrophic.papulosis:2:4867:0:16:1.00:1.00:0.11:0.20), (Microce phaly:2:4867:0:16:1.00:1.00:0.11:0.20), (Pituitary.enlargement:2:4867:0:16:1.00:1.00:0.11:0.20), (Prolapse:2:4867:0:16:1.00:1.00:0.11:0.20), (Rapidly.progressive.osteoarthritis:2:4867:0:16:1.00: 1.00:0.11:0.20), (Scrotal.pain:2:4867:0:16:1.00:1.00:0.11:0.20), (Superior.sagittal.sinus.thrombosi s:2:4867:0:16:1.00:1.00:0.11:0.20), (Tendon.operation:2:4867:0:16:1.00:1.00:0.11:0.20), (Artifici al.heart.implant:1:4868:0:16:1.00:1.00:0.06:0.11), (Atherectomy:1:4868:0:16:1.00:1.00:0.06:0.11), (Computerised.tomogram.abnormal:1:4868:0:16:1.00:1.00:0.06:0.11), (Convulsions.local:1:486 8:0:16:1.00:1.00:0.06:0.11), (Cystitis-like.symptom:1:4868:0:16:1.00:1.00:0.06:0.11), (Diabetic.arthropathy:1:4868:0:16:1.00:1.00:0.0 6:0.11), (Diabetic.gastroenteropathy:1:4868:0:16:1.00:1.00:0.06:0.11), (Ebola.disease:1:4868:0:16:1.00:1.00:0.06:0.11), (Ectopic.hormone.secretion:1:4868:0:16:1.00:1.00:0.06:0.11), (Genital.infe ction.male:1:4868:0:16:1.00:1.00:0.06:0.11), (Hepatitis.toxic:1:4868:0:16:1.00:1.00:0.06:0.11), (Hypochloraemia:1:4868:0:16:1.00:1.00:0.06:0.11), (Intestinal.anastomosis:1:4868:0:16:1.00:1.00: 0.06:0.11), (Intracranial.meningioma.malignant:1:4868:0:16:1.00:1.00:0.06:0.11), (Kidney.malfo rmation:1:4868:0:16:1.00:1.00:0.06:0.11), (Life.support:1:4868:0:16:1.00:1.00:0.06:0.11), (Lip.in jury:1:4868:0:16:1.00:1.00:0.06:0.11), (Mucosal.pain:1:4868:0:16:1.00:1.00:0.06:0.11), (Organic. brain.syndrome:1:4868:0:16:1.00:1.00:0.06:0.11), (Pericarditis.rheumatic:1:4868:0:16:1.00:1.00: 0.06:0.11), (Product.preparation.error:1:4868:0:16:1.00:1.00:0.06:0.11), (Renal.ischaemia:1:4868: 0:16:1.00:1.00:0.06:0.11), (Renal.vessel.disorder:1:4868:0:16:1.00:1.00:0.06:0.11), (Sadism:1:48 68:0:16:1.00:1.00:0.06:0.11), (Salmonella.sepsis:1:4868:0:16:1.00:1.00:0.06:0.11), (Silver-Russell.syndrome:1:4868:0:16:1.00:1.00:0.06:0.11), (Tendon.calcification:1:4868:0:16:1.00:1.00: 0.06:0.11), (Ammonia.decreased:0:4868:0:17:1.00:#NUM!:0.00:#NUM!), (Androgens.decreased: 0:4868:0:17:1.00:#NUM!:0.00:#NUM!), (Anticoagulation.drug.level.decreased:0:4868:0:17:1.00: #NUM!:0.00:#NUM!), (Aphrodisiac.therapy:0:4868:0:17:1.00:#NUM!:0.00:#NUM!), (Arteriove nous.fistula.site.infection:0:4868:0:17:1.00:#NUM!:0.00:#NUM!), (Bacterascites:0:4868:0:17:1.0 0:#NUM!:0.00:#NUM!), (Cutaneous.larva.migrans:0:4868:0:17:1.00:#NUM!:0.00:#NUM!), (Def ormity.of.orbit:0:4868:0:17:1.00:#NUM!:0.00:#NUM!), (Device.failure:0:4868:0:17:1.00:#NUM !:0.00:#NUM!), (Diabetic.coma:0:4868:0:17:1.00:#NUM!:0.00:#NUM!), (Factor.XI.deficiency:0: 4868:0:17:1.00:#NUM!:0.00:#NUM!), (Foreign.body:0:4868:0:17:1.00:#NUM!:0.00:#NUM!), (F ructose.intolerance:0:4868:0:17:1.00:#NUM!:0.00:#NUM!), (Glaucoma.drug.therapy:0:4868:0:1 7:1.00:#NUM!:0.00:#NUM!), (Glycogen.storage.disease.type.V:0:4868:0:17:1.00:#NUM!:0.00:#NUM!), (Gynaecological.examination.abnormal:0:4868:0:17:1.00:#NUM!:0.00:#NUM!), (Hango ver:0:4868:0:17:1.00:#NUM!:0.00:#NUM!), (Hypercreatininaemia:0:4868:0:17:1.00:#NUM!:0.0 0:#NUM!), (Hypothalamic.pituitary.adrenal.axis.suppression:0:4868:0:17:1.00:#NUM!:0.00:#N UM!), (Hysterosalpingo-oophorectomy:0:4868:0:17:1.00:#NUM!:0.00:#NUM!), (Iliotibial.band.syndrome:0:4868:0:17:1. 00:#NUM!:0.00:#NUM!), (Increased.ventricular.afterload:0:4868:0:17:1.00:#NUM!:0.00:#NUM !), (Lung.adenocarcinoma.stage.0:0:4868:0:17:1.00:#NUM!:0.00:#NUM!), (Mediastinal.abscess:0: 4868:0:17:1.00:#NUM!:0.00:#NUM!), (Microsporidia.infection:0:4868:0:17:1.00:#NUM!:0.00: #NUM!), (Mole.excision:0:4868:0:17:1.00:#NUM!:0.00:#NUM!), (Mycetoma.mycotic:0:4868:0: 17:1.00:#NUM!:0.00:#NUM!), (No.adverse.event:0:4868:0:17:1.00:#NUM!:0.00:#NUM!), (Non-secretory.adenoma.of.pituitary:0:4868:0:17:1.00:#NUM!:0.00:#NUM!), (Painful.ejaculation:0:48 68:0:17:1.00:#NUM!:0.00:#NUM!), (Pancreatic.necrosis:0:4868:0:17:1.00:#NUM!:0.00:#NUM!), (Paraspinal.abscess:0:4868:0:17:1.00:#NUM!:0.00:#NUM!), (Performance.fear:0:4868:0:17:1.0 0:#NUM!:0.00:#NUM!), (Peritoneal.effluent.abnormal:0:4868:0:17:1.00:#NUM!:0.00:#NUM!), (Placental.insufficiency:0:4868:0:17:1.00:#NUM!:0.00:#NUM!), (Premenstrual.pain:0:4868:0:17: 1.00:#NUM!:0.00:#NUM!), (Repetitive.speech:0:4868:0:17:1.00:#NUM!:0.00:#NUM!), (Respirat ory.alkalosis:0:4868:0:17:1.00:#NUM!:0.00:#NUM!), (Retinal.drusen:0:4868:0:17:1.00:#NUM!: 0.00:#NUM!), (Retroperitoneal.haemorrhage:0:4868:0:17:1.00:#NUM!:0.00:#NUM!), (Scarlet. fe ver:0:4868:0:17:1.00:#NUM!:0.00:#NUM!), (Senile.pruritus:0:4868:0:17:1.00:#NUM!:0.00:#NU M!), (Septic.arthritis.streptococcal:0:4868:0:17:1.00:#NUM!:0.00:#NUM!), (Somatic.delusion:0:4 868:0:17:1.00:#NUM!:0.00:#NUM!), (Stoma.site.infection:0:4868:0:17:1.00:#NUM!:0.00:#NU M!), (Suspiciousness:0:4868:0:17:1.00:#NUM!:0.00:#NUM!), (T-cell.chronic.lymphocytic.leukaemia:0:4868:0:17:1.00:#NUM!:0.00:#NUM!), (Tick-borne.viral.encephalitis:0:4868:0:17:1.00:#NUM!:0.00:#NUM!), (Tumour.marker.increased:0:48 68:0:17:1.00:#NUM!:0.00:#NUM!), (Up.and. down. phenomenon:0:4868:0:17:1.00:#NUM!:0.00: #NUM!), (Urethral.prolapse:0:4868:0:17:1.00:#NUM!:0.00:#NUM!), (Ventilation/perfusion.scan: 0:4868:0:17:1.00:#NUM!:0.00:#NUM!), (Vitamin.B6.abnormal:0:4868:0:17:1.00:#NUM!:0.00:#NUM!), (Congenital.retinoblastoma:12:4856:0:17:1.00:1.00:0.41:0.59), (Angioimmunoblastic.T-cell.lymphoma.stage.IV:11:4857:0:17:1.00:1.00:0.39:0.56), (Granulomatous.dermatitis:11:4857:0: 17:1.00:1.00:0.39:0.56), (Enteropathy-associated.T-cell.lymphoma:10:4858:0:17:1.00:1.00:0.37:0.54), (Macular.ischaemia:8:4860:0:17:1.00:1.00:0.3 2:0.48), (Testicular.germ.cell.cancer:7:4861:0:17:1.00:1.00:0.29:0.45), (Heart.and.lung.transplant: 6:4862:0:17:1.00:1.00:0.26:0.41), (Rectal.cancer.stage.II:6:4862:0:17:1.00:1.00:0.26:0.41), (Early.infantile.epileptic.encephalopathy. with.burst-suppression:5:4863:0:17:1.00:1.00:0.23:0.37), (Endometrial.cancer.stage.I:5:4863:0:17:1.00:1.00: 0.23:0.37), (Gastric.adenoma:5:4863:0:17:1.00:1.00:0.23:0.37), (Graft.versus.host.disease.in.eye: 5:4863:0:17:1.00:1.00:0.23:0.37), (Kyphoscoliosis:5:4863:0:17:1.00:1.00:0.23:0.37), (Gait.apraxi a:4:4864:0:17:1.00:1.00:0.19:0.32), (Hypofibrinogenaemia:4:4864:0:17:1.00:1.00:0.19:0.32), (Inj ection.site.irritation:4:4864:0:17:1.00:1.00:0.19:0.32), (Pancreatic.carcinoma.stage.III:4:4864:0:1 7:1.00:1.00:0.19:0.32), (Pedal.pulse.decreased:4:4864:0:17:1.00:1.00:0.19:0.32), (Postpartum.diso rder:4:4864:0:17:1.00:1.00:0.19:0.32), (Pterygium:4:4864:0:17:1.00:1.00:0.19:0.32), (Reperfusion.injury:4:4864:0:17:1.00:1.00:0.19:0.32), (Therapy.responder:4:4864:0:17:1.00:1.00:0.19:0.32), (Vertebral.lesion:4:4864:0:17:1.00:1.00:0.19:0.32), (Benign.neoplasm.of.bladder:3:4865:1:16:1.0 0:0.75:0.16:0.26), (Bladder.cancer.stage.0,.with.cancer.in.situ:3:4865:0:17:1.00:1.00:0.15:0.26), (Cardiac.infection:3:4865:0:17:1.00:1.00:0.15:0.26), (Epiglottic.carcinoma:3:4865:0:17:1.00:1.00: 0.15:0.26), (Epithelioid.sarcoma.metastatic:3:4865:0:17:1.00:1.00:0.15:0.26), (Gastrointestinal.isc haemia:3:4865:0:17:1.00:1.00:0.15:0.26), (Glottis.carcinoma:3:4865:0:17:1.00:1.00:0.15:0.26), (HLA-B*27.positive:3:4865:0:17:1.00:1.00:0.15:0.26), (Leukoerythroblastic.anaemia:3:4865:0:17:1.00: 1.00:0.15:0.26), (Ligament.operation:3:4865:0:17:1.00:1.00:0.15:0.26), (Meningitis.coccidioides: 3:4865:0:17:1.00:1.00:0.15:0.26), (Sensory.integrative.dysfunction:3:4865:0:17:1.00:1.00:0.15:0. 26), (Adrenalitis:2:4866:0:17:1.00:1.00:0.11:0.19), (Cardiac.septal.hypertrophy:2:4866:0:17:1.00: 1.00:0.11:0.19), (Contrast.media.deposition:2:4866:0:17:1.00:1.00:0.11:0.19), (Culture.stool.posit ive:2:4866:0:17:1.00:1.00:0.11:0.19), (Dark.circles.under.eyes:2:4866:0:17:1.00:1.00:0.11:0.19), (Erythrosis:2:4866:0:17:1.00:1.00:0.11:0.19), (Faecal.volume.increased:2:4866:0:17:1.00:1.00:0.1 1:0.19), (Finger.amputation:2:4866:0:17:1.00:1.00:0.11:0.19), (Genital.infection:2:4866:0:17:1.00: 1.00:0.11:0.19), (Hypophosphataemic.rickets:2:4866:0:17:1.00:1.00:0.11:0.19), (Lung.squamous. cell.carcinoma.stage.I:2:4866:0:17:1.00:1.00:0.11:0.19), (Malignant.melanoma.stage.I:2:4866:0:1 7:1.00:1.00:0.11:0.19), (Metastases.to.spinal.cord:2:4866:0:17:1.00:1.00:0.11:0.19), (Mitochondri al.enzyme.deficiency:2:4866:0:17:1.00:1.00:0.11:0.19), (Myotonia.congenita:2:4866:0:17:1.00:1. 00:0.11:0.19), (Optic.nerve.injury:2:4866:0:17:1.00:1.00:0.11:0.19), (Pathologic.myopia:2:4866:0: 17:1.00:1.00:0.11:0.19), (pH.urine:2:4866:0:17:1.00:1.00:0.11:0.19), (Precursor.T-lymphoblastic.lymphoma/leukaemia.recurrent:2:4866:0:17:1.00:1.00:0.11:0.19), (Pulmonary.arte rial.wedge.pressure:2:4866:0:17:1.00:1.00:0.11:0.19), (Reactive.gastropathy:2:4866:0:17:1.00:1. 00:0.11:0.19), (Retinoschisis:2:4866:0:17:1.00:1.00:0.11:0.19), (Transient.psychosis:2:4866:0:17: 1.00:1.00:0.11:0.19), (Uterine.atrophy:2:4866:0:17:1.00:1.00:0.11:0.19), (Vascular.access.compli cation:2:4866:0:17:1.00:1.00:0.11:0.19), (Abdominal.lymphadenopathy:1:4867:0:17:1.00:1.00:0. 06:0.11), (Acute.left.ventricular.failure:1:4867:0:17:1.00:1.00:0.06:0.11), (Acute.megakaryocytic. leukaemia:1:4867:0:17:1.00:1.00:0.06:0.11), (Anastomotic.ulcer:1:4867:0:17:1.00:1.00:0.06:0.11), (Angiogenesis.biomarker:1:4867:0:17:1.00:1.00:0.06:0.11), (Anti-thyroid.antibody.positive:1:4867:0:17:1.00:1.00:0.06:0.11), (Antimicrobial.susceptibility.test.resi stant:1:4867:0:17:1.00:1.00:0.06:0.11), (Aspiration.bronchial:1:4867:0:17:1.00:1.00:0.06:0.11), (Blood.caffeine:1:4867:0:17:1.00:1.00:0.06:0.11), (Drug.screen.positive:1:4867:0:17:1.00:1.00:0. 06:0.11), (Endometrial.sarcoma:1:4867:0:17:1.00:1.00:0.06:0.11), (Enzyme.level.abnormal:1:486 7:0:17:1.00:1.00:0.06:0.11), (Factor.I.deficiency:1:4867:0:17:1.00:1.00:0.06:0.11), (Fistula.of.sma ll.intestine:1:4867:0:17:1.00:1.00:0.06:0.11), (Hypercalcaemia.therapy:1:4867:0:17:1.00:1.00:0.0 6:0.11), (Hypertransaminasaemia:1:4867:0:17:1.00:1.00:0.06:0.11), (Hypoglycaemia.neonatal:1:4 867:0:17:1.00:1.00:0.06:0.11), (Hypogonadism.female:1:4867:0:17:1.00:1.00:0.06:0.11), (Incisio nal.drainage:1:4867:0:17:1.00:1.00:0.06:0.11), (Lown-Ganong-Levine.syndrome:1:4867:0:17:1.00:1.00:0.06:0.11), (Meningitis.06:0.11), (Meningitis.meningococcal:1:4867:0:17:1.00:1.00:0.06:0.11), (Personality.change.due.t o.a.general.medical.condition:1:4867:0:17:1.00:1.00:0.06:0.11), (Physical.disability:1:4867:0:17: 1.00:1.00:0.06:0.11), (Post.stroke.depression:1:4867:0:17:1.00:1.00:0.06:0.11), (Pressure.of.speec h:1:4867:0:17:1.00:1.00:0.06:0.11), (Radiosensitisation.therapy:1:4867:0:17:1.00:1.00:0.06:0.11), (Rheumatoid.scleritis:1:4867:0:17:1.00:1.00:0.06:0.11), (Sensory.processing.disorder:1:4867:0:1 7:1.00:1.00:0.06:0.11), (Spinal.cord.herniation:1:4867:0:17:1.00:1.00:0.06:0.11), (Virilism:1:4867: 0:17:1.00:1.00:0.06:0.11), (Wrist.surgery:1:4867:0:17:1.00:1.00:0.06:0.11), (Application.site.eryt hema:0:4867:0:18:1.00:#NUM!:0.00:#NUM!), (Blood.cholesterol.esterase.increased:0:4867:0:18: 1.00:#NUM!:0.00:#NUM!), (Carbamoyl.phosphate.synthetase.deficiency:0:4867:0:18:1.00:#NU M!:0.00:#NUM!), (Central.obesity:0:4867:0:18:1.00:#NUM!:0.00:#NUM!), (Chronic.idiopathic.p ain.syndrome:0:4867:0:18:1.00:#NUM!:0.00:#NUM!), (Comeal.graft.rejection:0:4867:0:18:1.00: #NUM!:0.00:#NUM!), (Comeal.opacity:0:4867:0:18:1.00:#NUM!:0.00:#NUM!), (Erythroblast.c ount:0:4867:0:18:1.00:#NUM!:0.00:#NUM!), (Hepatic.infection.bacterial:0:4867:0:18:1.00:#NU M!:0.00:#NUM!), (Homicide:0:4867:0:18:1.00:#NUM!:0.00:#NUM!), (Hypersomnia-bulimia.syndrome:0:4867:0:18:1.00:#NUM!:0.00:#NUM!), (Internal.fixation.of.fracture:0:4867:0: 18:1.00:#NUM!:0.00:#NUM!), (Junctional.ectopic.tachycardia:0:4867:0:18:1.00:#NUM!:0.00:#NUM!), (Klebsiella.test.positive:0:4867:0:18:1.00:#NUM!:0.00:#NUM!), (Mycotoxicosis:0:4867: 0:18:1.00:#NUM!:0.00:#NUM!), (Neonatal.infection:0:4867:0:18:1.00:#NUM!:0.00:#NUM!), (N odular.rash:0:4867:0:18:1.00:#NUM!:0.00:#NUM!), (Non-alcoholic.fatty.liver:0:4867:0:18:1.00:#NUM!:0.00:#NUM!), (Oropharyngeal.spasm:0:4867:0:18: 1.00:#NUM!:0.00:#NUM!), (Post-thoracotomy.pain.syndrome:0:4867:0:18:1.00:#NUM!:0.00:#NUM!), (Product.reconstitution.issu e:0:4867:0:18:1.00:#NUM!:0.00:#NUM!), (Pubic.pain:0:4867:0:18:1.00:#NUM!:0.00:#NUM!), (Pulmonary.function.challenge.test:0:4867:0:18:1.00:#NUM!:0.00:#NUM!), (Reflexes.abnormal:0: 4867:0:18:1.00:#NUM!:0.00:#NUM!), (Sinoatrial.block:0:4867:0:18:1.00:#NUM!:0.00:#NUM!), (Skin.swelling:0:4867:0:18:1.00:#NUM!:0.00:#NUM!), (Tympanoplasty:0:4867:0:18:1.00:#NU M!:0.00:#NUM!), (Viral.load.increased:0:4867:0:18:1.00:#NUM!:0.00:#NUM!), (Viral.load. unde tectable:0:4867:0:18:1.00:#NUM!:0.00:#NUM!), (Vulvovaginal.adhesion:0:4867:0:18:1.00:#NU M!:0.00:#NUM!), (Wound.haemorrhage:0:4867:0:18:1.00:#NUM!:0.00:#NUM!), (HIV.infection. CDC.category.B:17:4850:0:18:1.00:1.00:0.49:0.65), (Small.intestine.adenocarcinoma:11:4856:0: 18:1.00:1.00:0.38:0.55), (Leukaemic.lymphoma:10:4857:0:18:1.00:1.00:0.36:0.53), (Natural.kille r-cell.leukaemia:10:4857:0:18:1.00:1.00:0.36:0.53), (Neutrophilic.dermatosis:9:4858:0:18:1.00:1.0 0:0.33:0.50), (Non-renal.cell.carcinoma.of.kidney:9:4858:0:18:1.00:1.00:0.33:0.50), (Ovarian.hyperstimulation.synd rome:8:4859:1:17:1.00:0.89:0.32:0.47), (Female.genital.tract.fistula:7:4860:0:18:1.00:1.00:0.28:0 0.44), (Fistula.discharge:6:4861:0:18:1.00:1.00:0.25:0.40), (Metastases.to.uterus:5:4862:0:18:1.00: 1.00:0.22:0.36), (Stomatococcal.infection:5:4862:0:18:1.00:1.00:0.22:0.36), (Antineutrophil.cytop lasmic.antibody.decreased:4:4863:0:18:1.00:1.00:0.18:0.31), (Dyspnoea.at.rest:4:4863:0:18:1.00: 1.00:0.18:0.31), (Ejection.fraction.abnormal:4:4863:0:18:1.00:1.00:0.18:0.31), (Geotrichum.infect ion:4:4863:0:18:1.00:1.00:0.18:0.31), (Hepatitis.chronic.active:4:4863:0:18:1.00:1.00:0.18:0.31), (Injection.site.discolouration:4:4863:0:18:1.00:1.00:0.18:0.31), (Injection.site.urticaria:4:4863:0: 18:1.00:1.00:0.18:0.31), (Lichenoid.keratosis:4:4863:0:18:1.00:1.00:0.18:0.31), (Retinal.tear:4:48 63:0:18:1.00:1.00:0.18:0.31), (Scrotal.infection:4:4863:0:18:1.00:1.00:0.18:0.31), (Anorectal.infe ction:3:4864:0:18:1.00:1.00:0.14:0.25), (Chest.wall.abscess:3:4864:0:18:1.00:1.00:0.14:0.25), (Co arctation.of.the.aorta:3:4864:0:18:1.00:1.00:0.14:0.25), (Extra-osseous.Ewing's.sarcoma.metastatic:3:4864:0:18:1.00:1.00:0.14:0.25), (Fat.embolism:3:4864:0:1 8:1.00:1.00:0.14:0.25), (Fibrillary.glomerulonephritis:3:4864:0:18:1.00:1.00:0.14:0.25), (Hepatitis.fulminant:3:4864:0:18:1.00:1.00:0.14:0.25), (Histamine.level:3:4864:0:18:1.00:1.00:0.14:0.25), (Lung.hyperinflation:3:4864:0:18:1.00:1.00:0.14:0.25), (Monoparesis:3:4864:0:18:1.00:1.00:0.14: 0.25), (Mucous.stools:3:4864:0:18:1.00:1.00:0.14:0.25), (Non-small.cell.lung.cancer.stage.I:3:4864:0:18:1.00:1.00:0.14:0.25), (Presumed.ocular.histoplasmosis. syndrome:3:4864:0:18:1.00:1.00:0.14:0.25), (Squamous.cell.carcinoma.of.the.vagina:3:4864:0:18: 1.00:1.00:0.14:0.25), (Adenoid.cystic.carcinoma.of.salivary.gland:2:4865:0:18:1.00:1.00:0.10:0. 18), (Arteriogram:2:4865:0:18:1.00:1.00:0.10:0.18), (Bacterial.translocation:2:4865:0:18:1.00:1.0 0:0.10:0.18), (Bladder.injury:2:4865:0:18:1.00:1.00:0.10:0.18), (Butterfly.rash:2:4865:0:18:1.00:1 0.00:0.10:0.18), (Cardiac.stress.test.abnormal:2:4865:0:18:1.00:1.00:0.10:0.18), (Congenital.ectode rmal.dysplasia:2:4865:0:18:1.00:1.00:0.10:0.18), (Endometrial.cancer.stage.III:2:4865:0:18:1.00: 1.00:0.10:0.18), (Gastroduodenal.haemorrhage:2:4865:0:18:1.00:1.00:0.10:0.18), (Hepatitis.C.RN A.positive:2:4865:0:18:1.00:1.00:0.10:0.18), (Impaired.driving.ability:2:4865:0:18:1.00:1.00:0.1 0:0.18), (Lagophthalmos:2:4865:0:18:1.00:1.00:0.10:0.18), (May-Thurner.syndrome:2:4865:0:18:1.00:1.00:0.10:0.18), (Oesophageal.injury:2:4865:0:18:1.00:1.00: 0.10:0.18), (Ovarian.epithelial.cancer.stage.IV:2:4865:0:18:1.00:1.00:0.10:0.18), (Prostatic.specifi c.antigen.decreased:2:4865:0:18:1.00:1.00:0.10:0.18), (Retinal.vein.thrombosis:2:4865:0:18:1.00: 1.00:0.10:0.18), (Sensitivity.to.weather.change:2:4865:0:18:1.00:1.00:0.10:0.18), (Suture.insertio n:2:4865:0:18:1.00:1.00:0.10:0.18), (Vascular.stent.stenosis:2:4865:0:18:1.00:1.00:0.10:0.18), (At rial.pressure:1:4866:0:18:1.00:1.00:0.05:0.10), (Blood.creatine.phosphokinase:1:4866:0:18:1.00: 1.00:0.05:0.10), (Bronchitis.haemophilus:1:4866:0:18:1.00:1.00:0.05:0.10), (Cardiotoxicity:1:486 6:0:18:1.00:1.00:0.05:0.10), (Chronic.tic.disorder:1:4866:0:18:1.00:1.00:0.05:0.10), (Congenital. HIV.infection:1:4866:0:18:1.00:1.00:0.05:0.10), (Cortisol.increased:1:4866:0:18:1.00:1.00:0.05:0 0.10), (Drop.attacks:1:4866:0:18:1.00:1.00:0.05:0.10), (Drug.withdrawal.syndrome.neonatal:1:486 6:0:18:1.00:1.00:0.05:0.10), (Electrocardiogram.T.wave.abnormal:1:4866:0:18:1.00:1.00:0.05:0. 10), (Frustration:1:4866:0:18:1.00:1.00:0.05:0.10), (Generalised.erythema:1:4866:0:18:1.00:1.00: 0.05:0.10), (Genital.infection.female:1:4866:0:18:1.00:1.00:0.05:0.10), (Intra-aortic.balloon.placement:1:4866:0:18:1.00:1.00:0.05:0.10), (Intraductal.papillary.mucinous.neopl asm:1:4866:0:18:1.00:1.00:0.05:0.10), (Monocytopenia:1:4866:0:18:1.00:1.00:0.05:0.10), (Muell er's.mixed.tumour:1:4866:0:18:1.00:1.00:0.05:0.10), (Nuclear.magnetic.resonance.imaging.abnor mal:1:4866:0:18:1.00:1.00:0.05:0.10), (Organ.preservation:1:4866:0:18:1.00:1.00:0.05:0.10), (Ox ygen.consumption.increased:1:4866:0:18:1.00:1.00:0.05:0.10), (Pancreatic.mass:1:4866:0:18:1.0 0:1.00:0.05:0.10), (Pharyngeal.hypoaesthesia:1:4866:0:18:1.00:1.00:0.05:0.10), (Retinal.deposits: 1:4866:0:18:1.00:1.00:0.05:0.10), (Spherocytic.anaemia:1:4866:0:18:1.00:1.00:0.05:0.10), (Spinal meningioma.malignant:1:4866:0:18:1.00:1.00:0.05:0.10), (Spinal.nerve.stimulator.implantation: 1:4866:0:18:1.00:1.00:0.05:0.10), (Sympathetic.nerve.injury:1:4866:0:18:1.00:1.00:0.05:0.10), (T halassaemia.sickle.cell:1:4866:0:18:1.00:1.00:0.05:0.10), (Thyroid.adenoma.removal:1:4866:0:18: 1.00:1.00:0.05:0.10), (Urine.potassium:1:4866:0:18:1.00:1.00:0.05:0.10), (Wound.closure:1:4866: 0:18:1.00:1.00:0.05:0.10), (Analgesic.drug.level:0:4866:0:19:1.00:#NUM!:0.00:#NUM!), (Aneur ysm.ruptured:0:4866:0:19:1.00:#NUM!:0.00:#NUM!), (Bandaemia:0:4866:0:19:1.00:#NUM!:0.0 0:#NUM!), (Breast.angiosarcoma.metastatic:0:4866:0:19:1.00:#NUM!:0.00:#NUM!), (Brief.psyc hotic.disorder.with.marked.stressors:0:4866:0:19:1.00:#NUM!:0.00:#NUM!), (Capillary.fragility: 0:4866:0:19:1.00:#NUM!:0.00:#NUM!), (Carnitine.decreased:0:4866:0:19:1.00:#NUM!:0.00:#N UM!), (Cellulitis.streptococcal:0:4866:0:19:1.00:#NUM!:0.00:#NUM!), (Cholangiolitis:0:4866:0: 19:1.00:#NUM!:0.00:#NUM!), (Congenital.cytomegalovirus.infection:0:4866:0:19:1.00:#NUM!: 0.00:#NUM!), (Cortical.visual.impairment:0:4866:0:19:1.00:#NUM!:0.00:#NUM!), (Culture.nega tive:0:4866:0:19:1.00:#NUM!:0.00:#NUM!), (Disinhibition:0:4866:0:19:1.00:#NUM!:0.00:#NU M!), (Disorder.of.sex.development:0:4866:0:19:1.00:#NUM!:0.00:#NUM!), (Dystonic.tremor:0:4 866:0:19:1.00:#NUM!:0.00:#NUM!), (Gaucher's.disease.type.II:0:4866:0:19:1.00:#NUM!:0.00:#NUM!), (Gonadotrophin.releasing.hormone.deficiency:0:4866:0:19:1.00:#NUM!:0.00:#NUM!), (Hepatic.cyst.infection:0:4866:0:19:1.00:#NUM!:0.00:#NUM!), (Hyperuricosuria:0:4866:0:19:1.0 0:#NUM!:0.00:#NUM!), (In.vivo.gene.therapy:0:4866:0:19:1.00:#NUM!:0.00:#NUM!), (Jaundic e.cholestatic:0:4866:0:19:1.00:#NUM!:0.00:#NUM!), (Lesion.excision:0:4866:0:19:1.00:#NUM!: 0.00:#NUM!), (Mediastinal.operation:0:4866:0:19:1.00:#NUM!:0.00:#NUM!), (Naegleria.infecti on:0:4866:0:19:1.00:#NUM!:0.00:#NUM!), (Nasal.cavity.packing:0:4866:0:19:1.00:#NUM!:0.00: #NUM!), (Negativism:0:4866:0:19:1.00:#NUM!:0.00:#NUM!), (Normocytic.anaemia:0:4866:0:1 9:1.00:#NUM!:0.00:#NUM!), (Oesophageal.dilatation:0:4866:0:19:1.00:#NUM!:0.00:#NUM!), (Post.cholecystectomy.syndrome:0:4866:0:19:1.00:#NUM!:0.00:#NUM!), (Post. procedural. swelli ng:0:4866:0:19:1.00:#NUM!:0.00:#NUM!), (Product.quality.issue:0:4866:0:19:1.00:#NUM!:0.00: #NUM!), (Radiculitis.lumbosacral:0:4866:0:19:1.00:#NUM!:0.00:#NUM!), (Salivary.gland. muc ocoele:0:4866:0:19:1.00:#NUM!:0.00:#NUM!), (Stoma.site.erythema:0:4866:0:19:1.00:#NUM!: 0.00:#NUM!), (Subclavian.steal.syndrome:0:4866:0:19:1.00:#NUM!:0.00:#NUM!), (Superior.ven a.cava.occlusion:0:4866:0:19:1.00:#NUM!:0.00:#NUM!), (Thyroid.cancer.recurrent:0:4866:0:19: 1.00:#NUM!:0.00:#NUM!), (Tri-iodothyronine.free.decreased:0:4866:0:19:1.00:#NUM!:0.00:#NUM!), (Tubulisation.nerve.repair: 0:4866:0:19:1.00:#NUM!:0.00:#NUM!), (Tumour.flare:0:4866:0:19:1.00:#NUM!:0.00:#NUM!), (Urine.uric.acid.increased:0:4866:0:19:1.00:#NUM!:0.00:#NUM!), (Urodynamics.measurement:0: 4866:0:19:1.00:#NUM!:0.00:#NUM!), (Vulval.oedema:0:4866:0:19:1.00:#NUM!:0.00:#NUM!), (Wernicke's.encephalopathy:0:4866:0:19:1.00:#NUM!:0.00:#NUM!), (Wound.secretion:0:4866:0: 19:1.00:#NUM!:0.00:#NUM!), (Chronic.cutaneous.lupus.erythematosus:11:4855:0:19:1.00:1.00: 0.37:0.54), (Hodgkin's.disease.nodular.sclerosis.stage.IV:11:4855:0:19:1.00:1.00:0.37:0.54), (Dif fuse.vasculitis:9:4857:0:19:1.00:1.00:0.32:0.49), (Epstein-Barr.virus.test.positive:8:4858:0:19:1.00:1.00:0.30:0.46), (Exposure.via.partner:7:4859:0:19:1.00: 1.00:0.27:0.42), (Hypertensive.encephalopathy:6:4860:0:19:1.00:1.00:0.24:0.39), (Monarthritis:6: 4860:0:19:1.00:1.00:0.24:0.39), (Tuberculoma.of.central.nervous.system:6:4860:0:19:1.00:1.00:0 0.24:0.39), (Acquired.haemophilia.with.anti.FVIII,.XI,.or.XIII:5:4861:0:19:1.00:1.00:0.21:0.34), (Alagille.syndrome:5:4861:0:19:1.00:1.00:0.21:0.34), (Genital.ulceration:5:4861:0:19:1.00:1.00:0. 21:0.34), (Haemoglobinopathy:5:4861:0:19:1.00:1.00:0.21:0.34), (Infusion.site.pain:5:4861:0:19: 1.00:1.00:0.21:0.34), (Pulmonary.vasculitis:5:4861:0:19:1.00:1.00:0.21:0.34), (Angiofibroma:4:4 862:0:19:1.00:1.00:0.17:0.30), (Human.herpes.virus.6.serology.positive:4:4862:0:19:1.00:1.00:0. 17:0.30), (Non-small.cell.lung.cancer.stage.0:4:4862:0:19:1.00:1.00:0.17:0.30), (Rectal.lesion:4:4862:0:19:1.00: 1.00:0.17:0.30), (Reversible.cerebral.vasoconstriction.syndrome:4:4862:0:19:1.00:1.00:0.17:0.30), (Acanthamoeba.infection:3:4863:0:19:1.00:1.00:0.14:0.24), (Adenocarcinoma.of.salivary.gland: 3:4863:0:19:1.00:1.00:0.14:0.24), (Allergy.to.metals:3:4863:0:19:1.00:1.00:0.14:0.24), (Complem ent.factor.decreased:3:4863:0:19:1.00:1.00:0.14:0.24), (Congenital.pulmonary.artery.anomaly:3:4 863:0:19:1.00:1.00:0.14:0.24), (Daydreaming:3:4863:0:19:1.00:1.00:0.14:0.24), (Dilatation.ventri cular:3:4863:0:19:1.00:1.00:0.14:0.24), (Female.sex.hormone.level.abnormal:3:4863:0:19:1.00:1. 00:0.14:0.24), (Gliomatosis.cerebri:3:4863:0:19:1.00:1.00:0.14:0.24), (Plague:3:4863:0:19:1.00:1. 00:0.14:0.24), (Radical.prostatectomy:3:4863:0:19:1.00:1.00:0.14:0.24), (Tuberculosis.of.intratho racic.lymph.nodes:3:4863:1:18:1.00:0.75:0.14:0.24), (Viral.test.positive:3:4863:1:18:1.00:0.75:0. 14:0.24), (Allergy.alert.test.positive:2:4864:0:19:1.00:1.00:0.10:0.17), (Arthrogram:2:4864:0:19:1 0.00:1.00:0.10:0.17), (Bladder.obstruction:2:4864:0:19:1.00:1.00:0.10:0.17), (Blood.pressure.ortho static:2:4864:0:19:1.00:1.00:0.10:0.17), (Chronic.paroxysmal.hemicrania:2:4864:0:19:1.00:1.00: 0.10:0.17), (Congenital.abnormality.prophylaxis:2:4864:0:19:1.00:1.00:0.10:0.17), (Congenital. coagulopathy:2:4864:0:19:1.00:1.00:0.10:0.17), (Edentulous:2:4864:0:19:1.00:1.00:0.10:0.17), (Extremity. necrosis:2:4864:0:19:1.00:1.00:0.10:0.17), (Forearm.fracture:2:4864:0:19:1.00:1.00:0.10:0. 17), (Haematology.test.abnormal:2:4864:0:19:1.00:1.00:0.10:0.17), (Haemothorax:2:4864:0:19:1. 00:1.00:0.10:0.17), (Lentigo:2:4864:0:19:1.00:1.00:0.10:0.17), (Malignant.haemangiopericytoma. metastatic:2:4864:0:19:1.00:1.00:0.10:0.17), (Multimorbidity:2:4864:0:19:1.00:1.00:0.10:0.17), (Nasal.herpes:2:4864:0:19:1.00:1.00:0.10:0.17), (Peripheral.artery.angioplasty:2:4864:0:19:1.00:1 0.00:0.10:0.17), (Postpartum.state:2:4864:0:19:1.00:1.00:0.10:0.17), (Prevention.of.endometrial.hy perplasia:2:4864:0:19:1.00:1.00:0.10:0.17), (Reactive.attachment.disorder.of.infancy.or.early.chil dhood:2:4864:0:19:1.00:1.00:0.10:0.17), (Retinal.vascular.disorder:2:4864:0:19:1.00:1.00:0.10:0. 17), (Rheumatoid.factor.increased:2:4864:0:19:1.00:1.00:0.10:0.17), (Schnitzler's.syndrome:2:486 4:0:19:1.00:1.00:0.10:0.17), (Urine.calcium.increased:2:4864:0:19:1.00:1.00:0.10:0.17), (Vascula r.skin.disorder:2:4864:0:19:1.00:1.00:0.10:0.17), (Water.pollution:2:4864:0:19:1.00:1.00:0.10:0.1 7), (Advanced.sleep.phase:1:4865:0:19:1.00:1.00:0.05:0.10), (Allergic.oedema:1:4865:0:19:1.00: 1.00:0.05:0.10), (Anal.infection:1:4865:0:19:1.00:1.00:0.05:0.10), (Aortic.stent.insertion:1:4865:0: 19:1.00:1.00:0.05:0.10), (Astigmatism:1:4865:0:19:1.00:1.00:0.05:0.10), (Bacterial.tracheitis:1:4 865:0:19:1.00:1.00:0.05:0.10), (Bacteroides.infection:1:4865:0:19:1.00:1.00:0.05:0.10), (Cancer.i n.remission:1:4865:0:19:1.00:1.00:0.05:0.10), (Cardiovascular.somatic.symptom.disorder:1:4865:0:19:1.00:1.00:0.05:0.10), (Cell-mediated.immune.deficiency:1:4865:0:19:1.00:1.00:0.05:0.10), (Change.in.sustained.attention:1: 4865:0:19:1.00:1.00:0.05:0.10), (Cranial.nerve.injury:1:4865:0:19:1.00:1.00:0.05:0.10), (Cystic.ly mphangioma:1:4865:0:19:1.00:1.00:0.05:0.10), (Ear.infection.fungal:1:4865:0:19:1.00:1.00:0.05: 0.10), (Eyelid.exfoliation:1:4865:0:19:1.00:1.00:0.05:0.10), (Fatigue.management:1:4865:0:19:1. 00:1.00:0.05:0.10), (Hypoaldosteronism:1:4865:0:19:1.00:1.00:0.05:0.10), (Incorrect.dose.admini stered:1:4865:0:19:1.00:1.00:0.05:0.10), (Incorrect.drug.administration.duration:1:4865:0:19:1.00: 1.00:0.05:0.10), (Malignant.genitourinary.tract.neoplasm:1:4865:0:19:1.00:1.00:0.05:0.10), (Oed ematous.kidney:1:4865:0:19:1.00:1.00:0.05:0.10), (Pharyngeal.erythema:1:4865:0:19:1.00:1.00:0 0.05:0.10), (Renal.scan:1:4865:0:19:1.00:1.00:0.05:0.10), (Secondary.amyloidosis:1:4865:0:19:1.0 0:1.00:0.05:0.10), (Sensory.neuropathy.hereditary:1:4865:0:19:1.00:1.00:0.05:0.10), (Spermatozo a.abnormal:1:4865:0:19:1.00:1.00:0.05:0.10), (Staring:1:4865:0:19:1.00:1.00:0.05:0.10), (Thromb otic.stroke:1:4865:0:19:1.00:1.00:0.05:0.10), (Varicella.zoster.pneumonia:1:4865:0:19:1.00:1.00: 0.05:0.10), (Altered.visual.depth.perception:0:4865:0:20:1.00:#NUM!:0.00:#NUM!), (Anaphylact oid.reaction:0:4865:0:20:1.00:#NUM!:0.00:#NUM!), (Anaplastic.large.cell.lymphoma.T-.and.nul 1-cell.types.refractory:0:4865:0:20:1.00:#NUM!:0.00:#NUM!), (Angiotensin.converting.enzyme.in creased:0:4865:0:20:1.00:#NUM!:0.00:#NUM!), (Blood.testosterone.free.decreased:0:4865:0:20: 1.00:#NUM!:0.00:#NUM!), (Cardiac.imaging.procedure:0:4865:0:20:1.00:#NUM!:0.00:#NUM!), (Conjunctival.oedema:0:4865:0:20:1.00:#NUM!:0.00:#NUM!), (Dementia.of.the.Alzheimer's.ty pe,.with.delusions:0:4865:0:20:1.00:#NUM!:0.00:#NUM!), (Developmental.glaucoma:0:4865:0: 20:1.00:#NUM!:0.00:#NUM!), (Gastrointestinal.mucosal.exfoliation:0:4865:0:20:1.00:#NUM!:0. 00:#NUM!), (Iris.bombe:0:4865:0:20:1.00:#NUM!:0.00:#NUM!), (Multiple.injuries:0:4865:0:20: 1.00:#NUM!:0.00:#NUM!), (Muscle.swelling:0:4865:0:20:1.00:#NUM!:0.00:#NUM!), (Mycobac terial.peritonitis:0:4865:0:20:1.00:#NUM!:0.00:#NUM!), (Myocardial.oedema:0:4865:0:20:1.00: #NUM!:0.00:#NUM!), (Nail.discolouration:0:4865:0:20:1.00:#NUM!:0.00:#NUM!), (Pleural.diso rder:0:4865:0:20:1.00:#NUM!:0.00:#NUM!), (Pulmonary.function.test.normal:0:4865:0:20:1.00: #NUM!:0.00:#NUM!), (Red.blood.cell.enzymes. abnormal:0:4865:0:20:1.00:#NUM!:0.00:#NUM !), (Skin.angiosarcoma:0:4865:0:20:1.00:#NUM!:0.00:#NUM!), (Sleep.study:0:4865:0:20:1.00:#NUM!:0.00:#NUM!), (Spirometry:0:4865:0:20:1.00:#NUM!:0.00:#NUM!), (Splenic.infection.fun gal:0:4865:0:20:1.00:#NUM!:0.00:#NUM!), (Subdural.haemorrhage:0:4865:0:20:1.00:#NUM!:0. 00:#NUM!), (Systemic.immune.activation:0:4865:0:20:1.00:#NUM!:0.00:#NUM!), (Tooth.demin eralisation:0:4865:0:20:1.00:#NUM!:0.00:#NUM!), (Trichomoniasis:0:4865:0:20:1.00:#NUM!:0. 00:#NUM!), (Tricuspid.valve.replacement:0:4865:0:20:1.00:#NUM!:0.00:#NUM!), (Ulnar.nerve. injury:0:4865:0:20:1.00:#NUM!:0.00:#NUM!), (Susac's.syndrome:14:4851:0:20:1.00:1.00:0.41:0 0.58), (Epileptic.encephalopathy:13:4852:0:20:1.00:1.00:0.39:0.57), (Autoimmune.neutropenia:9:4 856:0:20:1.00:1.00:0.31:0.47), (Coombs.positive.haemolytic.anaemia:9:4856:0:20:1.00:1.00:0.31: 0.47), (Eisenmenger's.syndrome:9:4856:0:20:1.00:1.00:0.31:0.47), (Alopecia.totalis:8:4857:0:20: 1.00:1.00:0.29:0.44), (Carbohydrate.antigen.15-3.increased:8:4857:0:20:1.00:1.00:0.29:0.44), (Chronic.allograft.nephropathy:8:4857:0:20:1.00:1. 00:0.29:0.44), (Disseminated.Bacillus.Calmette-Guerin.infection:8:4857:1:19:1.00:0.89:0.30:0.44), (Infectious.thyroiditis:8:4857:0:20:1.00:1.00: 0.29:0.44), (Antibody.test.positive:7:4858:0:20:1.00:1.00:0.26:0.41), (Autosomal.chromosome. anomaly:7:4858:0:20:1.00:1.00:0.26:0.41), (Hepatic.candidiasis:7:4858:0:20:1.00:1.00:0.26:0.41), (Post.stroke.epilepsy:7:4858:0:20:1.00:1.00:0.26:0.41), (Benign.familial.pemphigus:6:4859:0:20:1 0.00:1.00:0.23:0.38), (Biliary.neoplasm:6:4859:0:20:1.00:1.00:0.23:0.38), (Hypopyon:6:4859:0:20: 1.00:1.00:0.23:0.38), (Angiotensin.converting.enzyme.decreased:5:4860:0:20:1.00:1.00:0.20:0.33), (Carcinoid.tumour.of.the.appendix:5:4860:0:20:1.00:1.00:0.20:0.33), (Lymphadenectomy:5:486 0:0:20:1.00:1.00:0.20:0.33), (Metastases.to.rectum:5:4860:0:20:1.00:1.00:0.20:0.33), (Physical.ab use:5:4860:0:20:1.00:1.00:0.20:0.33), (C3.glomerulopathy:4:4861:0:20:1.00:1.00:0.17:0.29), (Det achment.of.retinal.pigment.epithelium:4:4861:0:20:1.00:1.00:0.17:0.29), (Pancreas.transplant.rejection: 4:4861:0:20:1.00:1.00:0.17:0.29), (Pericarditis.tuberculous:4:4861:0:20:1.00:1.00:0.17:0.29), (Aortic.aneurysm.repair:3:4862:0:20:1.00:1.00:0.13:0.23), (Chest.X-ray:3:4862:0:20:1.00:1.00:0.13:0.23), (Concomitant.disease.progression:3:4862:0:20:1.00:1.00:0. 13:0.23), (Gamma.interferon.therapy:3:4862:0:20:1.00:1.00:0.13:0.23), (Mesangioproliferative.gl omerulonephritis:3:4862:0:20:1.00:1.00:0.13:0.23), (Photodynamic.therapy:3:4862:0:20:1.00:1.0 0:0.13:0.23), (Spinal.cord.injury.thoracic:3:4862:0:20:1.00:1.00:0.13:0.23), (Stridor:3:4862:0:20:1 0.00:1.00:0.13:0.23), (Urethral.cancer.metastatic:3:4862:0:20:1.00:1.00:0.13:0.23), (Bone.formation. increased:2:4863:0:20:1.00:1.00:0.09:0.17), (Cardiac.murmur.functional:2:4863:0:20:1.00:1.00: 0.09:0.17), (Cerumen.removal:2:4863:0:20:1.00:1.00:0.09:0.17), (Chronic.pigmented.purpura:2:4 863:0:20:1.00:1.00:0.09:0.17), (Culture.wound.positive:2:4863:0:20:1.00:1.00:0.09:0.17), (Exudat ive.retinopathy:2:4863:0:20:1.00:1.00:0.09:0.17), (Hypocarnitinaemia:2:4863:0:20:1.00:1.00:0.0 9:0.17), (Lactation.disorder:2:4863:0:20:1.00:1.00:0.09:0.17), (Liver.scan:2:4863:0:20:1.00:1.00: 0.09:0.17), (Marginal.zone.lymphoma.refractory:2:4863:0:20:1.00:1.00:0.09:0.17), (Occupational. therapy:2:4863:0:20:1.00:1.00:0.09:0.17), (Oropharyngeal.cancer.recurrent:2:4863:0:20:1.00:1.0 0:0.09:0.17), (Physical.examination:2:4863:0:20:1.00:1.00:0.09:0.17), (Pulmonary.arterial.pressur e.abnormal:2:4863:0:20:1.00:1.00:0.09:0.17), (Rectal.discharge:2:4863:0:20:1.00:1.00:0.09:0.17), (Rectal.fissure:2:4863:0:20:1.00:1.00:0.09:0.17), (Smear.cervix.abnormal:2:4863:0:20:1.00:1.00: 0.09:0.17), (Viral.haemorrhagic.cystitis:2:4863:0:20:1.00:1.00:0.09:0.17), (Vth.nerve.injury:2:486 3:0:20:1.00:1.00:0.09:0.17), (Abdominal.wall.abscess:1:4864:0:20:1.00:1.00:0.05:0.09), (Bone.for mation.decreased:1:4864:0:20:1.00:1.00:0.05:0.09), (Brain.contusion:1:4864:0:20:1.00:1.00:0.05: 0.09), (Carcinoid.tumour.of.the.duodenum:1:4864:0:20:1.00:1.00:0.05:0.09), (Cardiac.function.di sturbance.postoperative:1:4864:0:20:1.00:1.00:0.05:0.09), (Cardiac.rehabilitation.therapy:1:4864: 0:20:1.00:1.00:0.05:0.09), (Computerised.tomogram.coronary.artery:1:4864:0:20:1.00:1.00:0.05: 0.09), (Endolymphatic.hydrops:1:4864:0:20:1.00:1.00:0.05:0.09), (Eosinophilic.cystitis:1:4864:0: 20:1.00:1.00:0.05:0.09), (Eosinophilic.pneumonia.chronic:1:4864:0:20:1.00:1.00:0.05:0.09), (Flui d.intake.reduced:1:4864:0:20:1.00:1.00:0.05:0.09), (Heart.sounds.abnormal:1:4864:0:20:1.00:1.0 0:0.05:0.09), (Hyperchylomicronaemia:1:4864:0:20:1.00:1.00:0.05:0.09), (Hypoperfusion:1:4864: 0:20:1.00:1.00:0.05:0.09), (Incision.site.haemorrhage:1:4864:0:20:1.00:1.00:0.05:0.09), (Listless: 1:4864:0:20:1.00:1.00:0.05:0.09), (Malignant.melanoma.of.sites.other.than.skin:1:4864:0:20:1.00: 1.00:0.05:0.09), (Meningitis.streptococcal:1:4864:0:20:1.00:1.00:0.05:0.09), (Neuroleptic-induced.deficit.syndrome:1:4864:0:20:1.00:1.00:0.05:0.09), (Paediatric.autoimmune.neuropsychi atric.disorders.associated.with.streptococcal.infection:1:4864:0:20:1.00:1.00:0.05:0.09), (Plasma. cell.disorder:1:4864:0:20:1.00:1.00:0.05:0.09), (Red.man.syndrome:1:4864:0:20:1.00:1.00:0.05:0 0.09), (Renin-angiotensin.system.inhibition:1:4864:0:20:1.00:1.00:0.05:0.09), (Stereotypy:1:4864:0:20:1.00:1.0 0:0.05:0.09), (Traumatic.haemorrhage:1:4864:0:20:1.00:1.00:0.05:0.09), (Adrenoleukodystrophy: 0:4864:0:21:1.00:#NUM!:0.00:#NUM!), (Allergic.reaction.to.excipient:0:4864:0:21:1.00:#NUM!: 0.00:#NUM!), (Angiosclerosis:0:4864:0:21:1.00:#NUM!:0.00:#NUM!), (Aortic.valve.calcificatio n:0:4864:0:21:1.00:#NUM!:0.00:#NUM!), (Argininosuccinate.lyase.deficiency:0:4864:0:21:1.00: #NUM!:0.00:#NUM!), (Arthritis.fungal:0:4864:0:21:1.00:#NUM!:0.00:#NUM!), (Blood.aldoster one.increased:0:4864:0:21:1.00:#NUM!:0.00:#NUM!), (Blood.creatine.phosphokinase.increased: 0:4864:0:21:1.00:#NUM!:0.00:#NUM!), (Body.dysmorphic.disorder:0:4864:0:21:1.00:#NUM!:0. 00:#NUM!), (Carbohydrate.metabolism.disorder:0:4864:0:21:1.00:#NUM!:0.00:#NUM!), (Coagu lation.time.abnormal:0:4864:0:21:1.00:#NUM!:0.00:#NUM!), (Complement.fixation.abnormal:0: 4864:0:21:1.00:#NUM!:0.00:#NUM!), (Cytomegalovirus.immunisation:0:4864:0:21:1.00:#NUM !:0.00:#NUM!), (Developmental.hip.dysplasia:0:4864:0:21:1.00:#NUM!:0.00:#NUM!), (Feeling. of.despair:0:4864:0:21:1.00:#NUM!:0.00:#NUM!), (Gastric.mucosa. erythema:0:4864:0:21:1.00: #NUM!:0.00:#NUM!), (Hyposomnia:0:4864:0:21:1.00:#NUM!:0.00:#NUM!), (Inflammation.of. wound:0:4864:0:21:1.00:#NUM!:0.00:#NUM!), (Joint.instability:0:4864:0:21:1.00:#NUM!:0.00: #NUM!), (Lymphangitis:0:4864:0:21:1.00:#NUM!:0.00:#NUM!), (Myotonic.dystrophy:0:4864:0: 21:1.00:#NUM!:0.00:#NUM!), (Optic.atrophy:0:4864:0:21:1.00:#NUM!:0.00:#NUM!), (Oral.neo plasm:0:4864:0:21:1.00:#NUM!:0.00:#NUM!), (Perihepatic.abscess:0:4864:0:21:1.00:#NUM!:0. 00:#NUM!), (Phimosis:0:4864:0:21:1.00:#NUM!:0.00:#NUM!), (Repetitive.strain.injury:0:4864: 0:21:1.00:#NUM!:0.00:#NUM!), (Thought.blocking:0:4864:0:21:1.00:#NUM!:0.00:#NUM!), (Tr acheitis.obstructive:0:4864:0:21:1.00:#NUM!:0.00:#NUM!), (Tri-iodothyronine.uptake.abnormal:0:4864:0:21:1.00:#NUM!:0.00:#NUM!), (Tumour.ulceration:0:4 864:0:21:1.00:#NUM!:0.00:#NUM!), (Urethral.caruncle:0:4864:0:21:1.00:#NUM!:0.00:#NUM!), (Vitreous.opacities:0:4864:0:21:1.00:#NUM!:0.00:#NUM!), (Vulvar.erosion:0:4864:0:21:1.00:#NUM!:0.00:#NUM!), (Serous.cystadenocarcinoma.ovary:11:4853:1:20:1.00:0.92:0.35:0.51), (Lup us.cystitis:10:4854:0:21:1.00:1.00:0.32:0.49), (Mediastinum.neoplasm:8:4856:0:21:1.00:1.00:0.2 8:0.43), (Prevention.of.premature.ovulation:8:4856:1:20:1.00:0.89:0.29:0.43), (Congenital.syphilit ic.osteochondritis:7:4857:0:21:1.00:1.00:0.25:0.40), (Paraneoplastic.syndrome:7:4857:0:21:1.00: 1.00:0.25:0.40), (Pinealoblastoma:7:4857:0:21:1.00:1.00:0.25:0.40), (Planning.to.become.pregnan t:7:4857:0:21:1.00:1.00:0.25:0.40), (Nodular.vasculitis:6:4858:0:21:1.00:1.00:0.22:0.36), (Ocular. sarcoidosis:6:4858:0:21:1.00:1.00:0.22:0.36), (Complication.of.pregnancy:5:4859:0:21:1.00:1.00: 0.19:0.32), (Congenital.diaphragmatic.hernia:5:4859:0:21:1.00:1.00:0.19:0.32), (Congenital.pulm onary.valve.atresia:5:4859:0:21:1.00:1.00:0.19:0.32), (Gastrointestinal.stoma.output.increased:5: 4859:0:21:1.00:1.00:0.19:0.32), (Intestinal.stenosis:5:4859:0:21:1.00:1.00:0.19:0.32), (Metastases.to.eye:5:4859:0:21:1.00:1.00:0.19:0.32), (Microangiopathy:5:4859:0:21:1.00:1.00:0.19:0.32), (Po st.transfusion.purpura:5:4859:0:21:1.00:1.00:0.19:0.32), (Subclavian.artery.thrombosis:5:4859:0: 21:1.00:1.00:0.19:0.32), (Alcohol.rehabilitation:4:4860:0:21:1.00:1.00:0.16:0.28), (Alport's.syndr ome:4:4860:0:21:1.00:1.00:0.16:0.28), (Anovulatory.cycle:4:4860:0:21:1.00:1.00:0.16:0.28), (Fe male.reproductive.neoplasm:4:4860:0:21:1.00:1.00:0.16:0.28), (Goodpasture's.syndrome:4:4860: 0:21:1.00:1.00:0.16:0.28), (Herpes.simplex.meningitis:4:4860:0:21:1.00:1.00:0.16:0.28), (Immun oglobulin.G4.related.sclerosing.disease:4:4860:0:21:1.00:1.00:0.16:0.28), (Macular.cyst:4:4860:0: 21:1.00:1.00:0.16:0.28), (Myxofibrosarcoma:4:4860:0:21:1.00:1.00:0.16:0.28), (Retinal.telangiec tasia:4:4860:0:21:1.00:1.00:0.16:0.28), (Undifferentiated.nasopharyngeal.carcinoma:4:4860:0:21: 1.00:1.00:0.16:0.28), (Acne.conglobata:3:4861:0:21:1.00:1.00:0.13:0.22), (Chronic.eosinophilic.le ukaemia:3:4861:0:21:1.00:1.00:0.13:0.22), (Hair.transplant:3:4861:0:21:1.00:1.00:0.13:0.22), (Ma cular.detachment:3:4861:0:21:1.00:1.00:0.13:0.22), (Puberty:3:4861:0:21:1.00:1.00:0.13:0.22), (S uperficial.spreading.melanoma.stage.unspecified:3:4861:0:21:1.00:1.00:0.13:0.22), (Ureteric.can cer.metastatic:3:4861:0:21:1.00:1.00:0.13:0.22), (Urogenital.infection.bacterial:3:4861:0:21:1.00: 1.00:0.13:0.22), (Varicella.virus.test:3:4861:0:21:1.00:1.00:0.13:0.22), (Vitreous.detachment:3:48 61:0:21:1.00:1.00:0.13:0.22), (Amino.acid.level.abnormal:2:4862:0:21:1.00:1.00:0.09:0.16), (And ropause:2:4862:0:21:1.00:1.00:0.09:0.16), (Anticipatory.anxiety:2:4862:0:21:1.00:1.00:0.09:0.16), (Cowden's.disease:2:4862:0:21:1.00:1.00:0.09:0.16), (Diabetic.mononeuropathy:2:4862:0:21:1. 00:1.00:0.09:0.16), (Dural.tear:2:4862:0:21:1.00:1.00:0.09:0.16), (Eosinophil.count:2:4862:0:21:1 0.00:1.00:0.09:0.16), (Gastroenteritis.radiation:2:4862:0:21:1.00:1.00:0.09:0.16), (Glycogen.storag e.disease.type.I:2:4862:0:21:1.00:1.00:0.09:0.16), (Jejunal.ulcer:2:4862:0:21:1.00:1.00:0.09:0.16), (Luteal.phase.deficiency:2:4862:0:21:1.00:1.00:0.09:0.16), (Meningoencephalitis.amoebic:2:486 2:0:21:1.00:1.00:0.09:0.16), (Respiratory.arrest:2:4862:0:21:1.00:1.00:0.09:0.16), (Suppressed.lac tation:2:4862:0:21:1.00:1.00:0.09:0.16), (Uterine.polyp:2:4862:0:21:1.00:1.00:0.09:0.16), (Anapla stic.large.cell.lymphoma.T-.and.null-cell.types.stage.III:1:4863:0:21:1.00:1.00:0.05:0.09), (Auricular.swelling:1:4863:0:21:1.00:1.00:0 0.05:0.09), (B-lymphocyte.abnormalities:1:4863:0:21:1.00:1.00:0.05:0.09), (B-lymphocyte.count.abnormal:1:4863:0:21:1.00:1.00:0.05:0.09), (Chest.expansion.decreased:1:486 3:0:21:1.00:1.00:0.05:0.09), (Clear.cell.endometrial.carcinoma:1:4863:0:21:1.00:1.00:0.05:0.09), (Compulsive.hoarding:1:4863:0:21:1.00:1.00:0.05:0.09), (Concomitant.disease.aggravated:1:486 3:0:21:1.00:1.00:0.05:0.09), (Contact.lens.therapy:1:4863:0:21:1.00:1.00:0.05:0.09), (Crowned.de ns.syndrome:1:4863:0:21:1.00:1.00:0.05:0.09), (Follicle-stimulating.hormone.deficiency:1:4863:0:21:1.00:1.00:0.05:0.09), (Gastric.emptying.study:1:486 3:0:21:1.00:1.00:0.05:0.09), (Genital.burning.sensation:1:4863:0:21:1.00:1.00:0.05:0.09), (Hepati c.angiosarcoma:1:4863:0:21:1.00:1.00:0.05:0.09), (Heterotaxia:1:4863:0:21:1.00:1.00:0.05:0.09), (Infantile.asthma:1:4863:0:21:1.00:1.00:0.05:0.09), (Laryngeal.cancer.stage.III:1:4863:0:21:1.00: 1.00:0.05:0.09), (Leptospirosis:1:4863:0:21:1.00:1.00:0.05:0.09), (Loss.of.personal.independence. in.daily.activities:1:4863:0:21:1.00:1.00:0.05:0.09), (Malignant.fibrous.histiocytoma.metastatic:1: 4863:0:21:1.00:1.00:0.05:0.09), (Orchitis.noninfective:1:4863:0:21:1.00:1.00:0.05:0.09), (Panoph thalmitis:1:4863:0:21:1.00:1.00:0.05:0.09), (Parkinsonian.rest.tremor:1:4863:0:21:1.00:1.00:0.05: 0.09), (Pneumonectomy:1:4863:0:21:1.00:1.00:0.05:0.09), (Procedural.headache:1:4863:0:21:1.00: 1.00:0.05:0.09), (Sensorimotor.disorder:1:4863:0:21:1.00:1.00:0.05:0.09), (Social.avoidant.behav iour:1:4863:0:21:1.00:1.00:0.05:0.09), (Supine.hypertension:1:4863:0:21:1.00:1.00:0.05:0.09), (T hyroxine.therapy:1:4863:0:21:1.00:1.00:0.05:0.09), (Vitamin.K.decreased:1:4863:0:21:1.00:1.00: 0.05:0.09), (Achlorhydria:0:4863:0:22:1.00:#NUM!:0.00:#NUM!), (Adrenal.gland.injury:0:4863: 0:22:1.00:#NUM!:0.00:#NUM!), (Axillary.mass:0:4863:0:22:1.00:#NUM!:0.00:#NUM!), (Corne al.infection:0:4863:0:22:1.00:#NUM!:0.00:#NUM!), (Corrective.lens.user:0:4863:0:22:1.00:#NU M!:0.00:#NUM!), (Delusional.disorder,.persecutory.type:0:4863:0:22:1.00:#NUM!:0.00:#NUM!), (Enanthema:0:4863:0:22:1.00:#NUM!:0.00:#NUM!), (Epidural.lipomatosis:0:4863:0:22:1.00:#NUM!:0.00:#NUM!), (Familial.hypertriglyceridaemia:0:4863:0:22:1.00:#NUM!:0.00:#NUM!), (Hepatic.echinococciasis:0:4863:0:22:1.00:#NUM!:0.00:#NUM!), (Injection.site.extravasation:0:4 863:0:22:1.00:#NUM!:0.00:#NUM!), (Painful.respiration:0:4863:0:22:1.00:#NUM!:0.00:#NUM!), (Pneumonia.escherichia:0:4863:0:22:1.00:#NUM!:0.00:#NUM!), (Polydipsia:0:4863:0:22:1.00: #NUM!:0.00:#NUM!), (Pulmonary.calcification:0:4863:0:22:1.00:#NUM!:0.00:#NUM!), (Remna nt.gastritis:0:4863:0:22:1.00:#NUM!:0.00:#NUM!), (Retinal.operation:0:4863:0:22:1.00:#NUM!: 0.00:#NUM!), (Small.intestinal.resection:0:4863:0:22:1.00:#NUM!:0.00:#NUM!), (Strangulated. hernia:0:4863:0:22:1.00:#NUM!:0.00:#NUM!), (Total.lung.capacity.abnormal:0:4863:0:22:1.00: #NUM!:0.00:#NUM!), (Treponema.test.positive:0:4863:0:22:1.00:#NUM!:0.00:#NUM!), (Ultras ound. scan:0:4863:0:22:1.00:#NUM!:0.00:#NUM!), (Vessel.puncture.site.pain:0:4863:0:22:1.00:#NUM!:0.00:#NUM!), (Weaning.failure:0:4863:0:22:1.00:#NUM!:0.00:#NUM!), (Choroiditis:18: 4845:0:22:1.00:1.00:0.45:0.62), (HIV.test:17:4846:0:22:1.00:1.00:0.44:0.61), (Diffuse.large.B-cell.lymphoma.stage.I:14:4849:0:22:1.00:1.00:0.39:0.56), (HIV.infection.CDC.Group.IV.subgrou p.C1:8:4855:0:22:1.00:1.00:0.27:0.42), (Cytomegalovirus.test:7:4856:0:22:1.00:1.00:0.24:0.39), (Follicle.centre.lymphoma,.follicular.grade.I,.II,.III.recurrent:7:4856:0:22:1.00:1.00:0.24:0.39), (P eripheral.T-cell.lymphoma.unspecified.recurrent:7:4856:0:22:1.00:1.00:0.24:0.39), (Reflux.nephropathy:7:48 56:0:22:1.00:1.00:0.24:0.39), (Chronic.recurrent.multifocal.osteomyelitis:6:4857:0:22:1.00:1.00: 0.21:0.35), (Extra-osseous.Ewing's.sarcoma:6:4857:0:22:1.00:1.00:0.21:0.35), (Hepatitis.B.antigen.positive:6:4857: 0:22:1.00:1.00:0.21:0.35), (Cardiac.tamponade:5:4858:0:22:1.00:1.00:0.19:0.31), (Graft.thrombos is:5:4858:0:22:1.00:1.00:0.19:0.31), (Hodgkin's.disease.lymphocyte.depletion.type.refractory:5:4 858:0:22:1.00:1.00:0.19:0.31), (Lymphatic.system.neoplasm:5:4858:0:22:1.00:1.00:0.19:0.31), (Weight.fluctuation:5:4858:0:22:1.00:1.00:0.19:0.31), (Anticoagulation.drug.level.above.therapeu tic:4:4859:0:22:1.00:1.00:0.15:0.27), (Blast.cell.crisis:4:4859:0:22:1.00:1.00:0.15:0.27), (Hodgkin's.disease.stage.I:4:4859:0:22:1.00:1.00:0.15:0.27), (Mouth.swelling:4:4859:0:22:1.00:1.00:0.15: 0.27), (Parvovirus.infection:4:4859:0:22:1.00:1.00:0.15:0.27), (Scleral.oedema:4:4859:0:22:1.00: 1.00:0.15:0.27), (Urinary.bladder.haemorrhage:4:4859:0:22:1.00:1.00:0.15:0.27), (Appendicitis.p erforated:3:4860:0:22:1.00:1.00:0.12:0.21), (Asymptomatic.bacteriuria:3:4860:0:22:1.00:1.00:0.1 2:0.21), (Ballismus:3:4860:0:22:1.00:1.00:0.12:0.21), (Brain.malformation:3:4860:0:22:1.00:1.00: 0.12:0.21), (Endometrial.cancer.stage.IV:3:4860:0:22:1.00:1.00:0.12:0.21), (Male.sexual.dysfunct ion:3:4860:0:22:1.00:1.00:0.12:0.21), (Nerve.conduction.studies.abnormal:3:4860:0:22:1.00:1.00: 0.12:0.21), (Post.procedural.pulmonary.embolism:3:4860:0:22:1.00:1.00:0.12:0.21), (Subclavian. artery.occlusion:3:4860:0:22:1.00:1.00:0.12:0.21), (Toxic.shock.syndrome:3:4860:0:22:1.00:1.00:0.12:0.21), (Administration.site.thrombosis:2:4861:0:22:1.00:1.00:0.08:0.15), (Anomalous.pulmo nary.venous.connection:2:4861:0:22:1.00:1.00:0.08:0.15), (Biotin.deficiency:2:4861:0:22:1.00:1. 00:0.08:0.15), (Drug.withdrawal.convulsions:2:4861:0:22:1.00:1.00:0.08:0.15), (Epinephrine.incr eased:2:4861:0:22:1.00:1.00:0.08:0.15), (Gastrointestinal.erosion:2:4861:0:22:1.00:1.00:0.08:0.1 5), (Haemoglobinuria:2:4861:0:22:1.00:1.00:0.08:0.15), (Herpes.zoster.infection.neurological:2:4 861:0:22:1.00:1.00:0.08:0.15), (Hodgkin's.disease.nodular.sclerosis.stage.III:2:4861:0:22:1.00:1. 00:0.08:0.15), (Hyper.HDL.cholesterolaemia:2:4861:0:22:1.00:1.00:0.08:0.15), (Meningitis.herpe s:2:4861:0:22:1.00:1.00:0.08:0.15), (Oestradiol.abnormal:2:4861:0:22:1.00:1.00:0.08:0.15), (Phys ical.fitness.training:2:4861:0:22:1.00:1.00:0.08:0.15), (Post.stroke.seizure:2:4861:0:22:1.00:1.00: 0.08:0.15), (Retroperitoneal.mass:2:4861:0:22:1.00:1.00:0.08:0.15), (Rhonchi:2:4861:0:22:1.00:1. 00:0.08:0.15), (Adenoidal.hypertrophy:1:4862:0:22:1.00:1.00:0.04:0.08), (Animal.scratch:1:4862: 0:22:1.00:1.00:0.04:0.08), (Argininosuccinate.synthetase.deficiency:1:4862:0:22:1.00:1.00:0.04:0 0.08), (Bile.output.increased:1:4862:0:22:1.00:1.00:0.04:0.08), (Blood.immunoglobulin.E.decrease d:1:4862:0:22:1.00:1.00:0.04:0.08), (Breast.scan:1:4862:0:22:1.00:1.00:0.04:0.08), (Eyelid.margi n.crusting:1:4862:0:22:1.00:1.00:0.04:0.08), (Fear.of.disease:1:4862:0:22:1.00:1.00:0.04:0.08), (G astrointestinal.examination:1:4862:0:22:1.00:1.00:0.04:0.08), (Haemorrhagic.erosive.gastritis:1:4 862:0:22:1.00:1.00:0.04:0.08), (Hyperglobulinaemia:1:4862:0:22:1.00:1.00:0.04:0.08), (Impaired. work.ability:1:4862:0:22:1.00:1.00:0.04:0.08), (Lip.blister:1:4862:0:22:1.00:1.00:0.04:0.08), (Met astases.to.pituitary.gland:1:4862:0:22:1.00:1.00:0.04:0.08), (Myocardial.necrosis.marker:1:4862: 0:22:1.00:1.00:0.04:0.08), (Ovarian.germ.cell.embryonal.carcinoma.stage.III:1:4862:0:22:1.00:1. 00:0.04:0.08), (Penile.pain:1:4862:0:22:1.00:1.00:0.04:0.08), (Selenium.deficiency:1:4862:0:22:1. 00:1.00:0.04:0.08), (Acinetobacter.test.positive:0:4862:0:23:1.00:#NUM!:0.00:#NUM!), (Alcohol.problem:0:4862:0:23:1.00:#NUM!:0.00:#NUM!), (Ammonia:0:4862:0:23:1.00:#NUM!:0.00:#N UM!), (Bacterial.abdominal.infection:0:4862:0:23:1.00:#NUM!:0.00:#NUM!), (Bullous.lung.dise ase:0:4862:0:23:1.00:#NUM!:0.00:#NUM!), (Carbon.monoxide.poisoning:0:4862:0:23:1.00:#N UM!:0.00:#NUM!), (Cardiac.pacemaker.replacement:0:4862:0:23:1.00:#NUM!:0.00:#NUM!), (C ongenital.megacolon:0:4862:0:23:1.00:#NUM!:0.00:#NUM!), (Crystalluria:0:4862:0:23:1.00:#N UM!:0.00:#NUM!), (Cystitis.ulcerative:0:4862:0:23:1.00:#NUM!:0.00:#NUM!), (Ear.infection.ba cterial:0:4862:0:23:1.00:#NUM!:0.00:#NUM!), (Excessive.granulation.tissue:0:4862:0:23:1.00:#NUM!:0.00:#NUM!), (Exposure.to.mould:0:4862:0:23:1.00:#NUM!:0.00:#NUM!), (Eyelid.infect ion:0:4862:0:23:1.00:#NUM!:0.00:#NUM!), (Feelings.of.worthlessness:0:4862:0:23:1.00:#NUM !:0.00:#NUM!), (IgM.nephropathy:0:4862:0:23:1.00:#NUM!:0.00:#NUM!), (Imperforate.oesopha gus:0:4862:0:23:1.00:#NUM!:0.00:#NUM!), (Jugular.vein.occlusion:0:4862:0:23:1.00:#NUM!:0. 00:#NUM!), (Parasite.blood.test.positive:0:4862:0:23:1.00:#NUM!:0.00:#NUM!), (Prostate.ablati on:0:4862:0:23:1.00:#NUM!:0.00:#NUM!), (Thyroxine.abnormal:0:4862:0:23:1.00:#NUM!:0.00: #NUM!), (Upper.airway.obstruction:0:4862:0:23:1.00:#NUM!:0.00:#NUM!), (Urostomy:0:4862: 0:23:1.00:#NUM!:0.00:#NUM!), (Peripheral.blood.stem.cell.apheresis:19:4843:0:23:1.00:1.00:0. 45:0.62), (K-ras.gene.mutation:13:4849:0:23:1.00:1.00:0.36:0.53), (Uterine.neoplasm:13:4849:0:23:1.00:1.00: 0.36:0.53), (Erythema.annulare:9:4853:0:23:1.00:1.00:0.28:0.44), (Hodgkin's.disease.nodular.scle rosis.recurrent:8:4854:0:23:1.00:1.00:0.26:0.41), (Pachymeningitis:8:4854:0:23:1.00:1.00:0.26:0. 41), (Acute.generalised.exanthematous.pustulosis:7:4855:0:23:1.00:1.00:0.23:0.38), (Subacute.scl erosing.panencephalitis:7:4855:0:23:1.00:1.00:0.23:0.38), (Adrenal.androgen.deficiency:6:4856: 0:23:1.00:1.00:0.21:0.34), (Leishmaniasis:6:4856:0:23:1.00:1.00:0.21:0.34), (Pre-engraftment.immune.reaction:6:4856:0:23:1.00:1.00:0.21:0.34), (Renal.tubular.necrosis:6:4856:0: 23:1.00:1.00:0.21:0.34), (Mycobacterium.test.positive:5:4857:0:23:1.00:1.00:0.18:0.30), (Alloim munisation:4:4858:0:23:1.00:1.00:0.15:0.26), (Congenital.absence.of.bile.ducts:4:4858:0:23:1.00: 1.00:0.15:0.26), (Congenital.pulmonary.hypertension:4:4858:0:23:1.00:1.00:0.15:0.26), (Exophth almos:4:4858:0:23:1.00:1.00:0.15:0.26), (Gallbladder.pain:4:4858:0:23:1.00:1.00:0.15:0.26), (Hu man.T-cell.lymphotropic.virus.type.I.infection:4:4858:0:23:1.00:1.00:0.15:0.26), (Intracardiac.mass:4:48 58:0:23:1.00:1.00:0.15:0.26), (Ovarian.cancer.stage.I:4:4858:0:23:1.00:1.00:0.15:0.26), (Transferr in.saturation.decreased:4:4858:0:23:1.00:1.00:0.15:0.26), (Vocal.cord.inflammation:4:4858:0:23: 1.00:1.00:0.15:0.26), (Endometrial.hypertrophy:3:4859:0:23:1.00:1.00:0.12:0.21), (Finger.deform ity:3:4859:0:23:1.00:1.00:0.12:0.21), (Hepatic.enzyme.abnormal:3:4859:0:23:1.00:1.00:0.12:0.21), (Hypopharyngeal.cancer.recurrent:3:4859:0:23:1.00:1.00:0.12:0.21), (Kaposi's.varicelliform.eru ption:3:4859:0:23:1.00:1.00:0.12:0.21), (Malignant.oligodendroglioma:3:4859:0:23:1.00:1.00:0.1 2:0.21), (Positron.emission.tomogram.abnormal:3:4859:0:23:1.00:1.00:0.12:0.21), (Respiratory.tr act.haemorrhage:3:4859:0:23:1.00:1.00:0.12:0.21), (Biliary.anastomosis:2:4860:0:23:1.00:1.00:0. 08:0.15), (Blood.immunoglobulin.A.decreased:2:4860:0:23:1.00:1.00:0.08:0.15), (Bone.contusion: 2:4860:0:23:1.00:1.00:0.08:0.15), (Breast.hyperplasia:2:4860:0:23:1.00:1.00:0.08:0.15), (Cervicit is:2:4860:0:23:1.00:1.00:0.08:0.15), (Chronic.lymphocytic.leukaemia.stage.1:2:4860:0:23:1.00:1. 00:0.08:0.15), (Cyclic.neutropenia:2:4860:0:23:1.00:1.00:0.08:0.15), (Cytomegalovirus.oesophagi tis:2:4860:0:23:1.00:1.00:0.08:0.15), (Eczema.eyelids:2:4860:0:23:1.00:1.00:0.08:0.15), (Gynaec ological.examination:2:4860:0:23:1.00:1.00:0.08:0.15), (Infestation:2:4860:0:23:1.00:1.00:0.08:0 0.15), (Injection.site.mass:2:4860:0:23:1.00:1.00:0.08:0.15), (Intemational.normalised.ratio.fluctuation: 2:4860:0:23:1.00:1.00:0.08:0.15), (Intestinal.congestion:2:4860:0:23:1.00:1.00:0.08:0.15), (Le nticular.operation:2:4860:0:23:1.00:1.00:0.08:0.15), (Lipoprotein.deficiency:2:4860:0:23:1.00:1. 00:0.08:0.15), (Lymphocytic.infiltration:2:4860:0:23:1.00:1.00:0.08:0.15), (Malignant.palate.neoplasm: 2:4860:0:23:1.00:1.00:0.08:0.15), (Microvillous.inclusion.disease:2:4860:0:23:1.00:1.00:0.0 8:0.15), (Myringitis.bullous:2:4860:0:23:1.00:1.00:0.08:0.15), (Pancreatic.enzymes.increased:2:4 860:0:23:1.00:1.00:0.08:0.15), (Paranoid.personality.disorder:2:4860:0:23:1.00:1.00:0.08:0.15), (Radiation.myelopathy:2:4860:0:23:1.00:1.00:0.08:0.15), (Silent.myocardial.infarction:2:4860:0:2 3:1.00:1.00:0.08:0.15), (Single.functional.kidney:2:4860:0:23:1.00:1.00:0.08:0.15), (Stoma.site.ab scess:2:4860:0:23:1.00:1.00:0.08:0.15), (Transurethral.bladder.resection:2:4860:0:23:1.00:1.00:0. 08:0.15), (Base.excess.abnormal:1:4861:0:23:1.00:1.00:0.04:0.08), (Blood.fibrinogen.decreased:1: 4861:0:23:1.00:1.00:0.04:0.08), (Cardiac.valve.replacement.complication:1:4861:0:23:1.00:1.00: 0.04:0.08), (Clinical.trial.participant:1:4861:0:23:1.00:1.00:0.04:0.08), (Cryotherapy:1:4861:0:23: 1.00:1.00:0.04:0.08), (Cytogenetic.analysis:1:4861:0:23:1.00:1.00:0.04:0.08), (Disseminated.cyto megaloviral.infection:1:4861:0:23:1.00:1.00:0.04:0.08), (Gastroenteritis.bacterial:1:4861:0:23:1.0 0:1.00:0.04:0.08), (Hydraemia:1:4861:0:23:1.00:1.00:0.04:0.08), (Hypervitaminosis:1:4861:0:23: 1.00:1.00:0.04:0.08), (Incoherent:1:4861:0:23:1.00:1.00:0.04:0.08), (Joint.prosthesis.user:1:4861: 0:23:1.00:1.00:0.04:0.08), (Lateral.medullary.syndrome:1:4861:0:23:1.00:1.00:0.04:0.08), (Linear.IgA.disease:1:4861:0:23:1.00:1.00:0.04:0.08), (Metastases.to.bladder:1:4861:0:23:1.00:1.00:0.04: 0.08), (Nephrogenic.diabetes.insipidus:1:4861:0:23:1.00:1.00:0.04:0.08), (Pulmonary.haemosider osis:1:4861:0:23:1.00:1.00:0.04:0.08), (Radiotherapy.to.bone:1:4861:0:23:1.00:1.00:0.04:0.08), (Sebaceous.hyperplasia:1:4861:0:23:1.00:1.00:0.04:0.08), (Spondylitic.myelopathy:1:4861:0:23:1. 00:1.00:0.04:0.08), (Tooth.injury:1:4861:0:23:1.00:1.00:0.04:0.08), (Ureteral.stent.insertion:1:486 1:0:23:1.00:1.00:0.04:0.08), (Weil's.disease:1:4861:0:23:1.00:1.00:0.04:0.08), (Actinic.cheilitis:0: 4861:0:24:1.00:#NUM!:0.00:#NUM!), (Anaesthetic.complication.neurological:0:4861:0:24:1.00: #NUM!:0.00:#NUM!), (Ascariasis:0:4861:0:24:1.00:#NUM!:0.00:#NUM!), (Bed.bug.infestation: 0:4861:0:24:1.00:#NUM!:0.00:#NUM!), (Bladder.dilatation:0:4861:0:24:1.00:#NUM!:0.00:#NU M!), (Blood.culture:0:4861:0:24:1.00:#NUM!:0.00:#NUM!), (Chondroplasty:0:4861:0:24:1.00:#NUM!:0.00:#NUM!), (Ebstein's.anomaly:0:4861:0:24:1.00:#NUM!:0.00:#NUM!), (Flat.affect:0:4 861:0:24:1.00:#NUM!:0.00:#NUM!), (Floppy.eyelid.syndrome:0:4861:0:24:1.00:#NUM!:0.00:#NUM!), (Gastrointestinal.malformation:0:4861:0:24:1.00:#NUM!:0.00:#NUM!), (Hand-foot-and-mouth.disease:0:4861:0:24:1.00:#NUM!:0.00:#NUM!), (Hepatic.hydrothorax:0:4861:0:24:1.00:#NUM!:0.00:#NUM!), (Hypoaesthesia.oral:0:4861:0:24:1.00:#NUM!:0.00:#NUM!), (Late.onset. hypogonadism. syndrome:0:4861:0:24:1.00:#NUM!:0.00:#NUM!), (Pasteurella.infection:0:4861:0: 24:1.00:#NUM!:0.00:#NUM!), (Peripheral.artery.aneurysm:0:4861:0:24:1.00:#NUM!:0.00:#NU M!), (Prostate.cancer.stage.0:0:4861:0:24:1.00:#NUM!:0.00:#NUM!), (Prostatic.abscess:0:4861:0: 24:1.00:#NUM!:0.00:#NUM!), (Wheelchair.user:0:4861:0:24:1.00:#NUM!:0.00:#NUM!), (Sever e.myoclonic.epilepsy.of.infancy:15:4846:1:23:1.00:0.94:0.39:0.56), (Juvenile.chronic.myelomon ocytic.leukaemia:13:4848:0:24:1.00:1.00:0.35:0.52), (Metastases.to.muscle:12:4849:0:24:1.00:1. 00:0.33:0.50), (Non-Hodgkin's.lymphoma.metastatic:9:4852:0:24:1.00:1.00:0.27:0.43), (Testicular.neoplasm:9:4852:0:24:1.00:1.00:0.27:0.43), (Germ.cell.cancer.metastatic:8:4853:0:24:1.00:1.00:0.25:0.40), (Overlap.syndrome:8:4853:0:24:1.00:1.00:0.25:0.40), (Alopecia.universalis:7:4854:0:24:1.00:1.00:0.23:0. 37), (Precursor.B-lymphoblastic.lymphoma.recurrent:7:4854:0:24:1.00:1.00:0.23:0.37), (Sick.sinus.syndrome:7:48 54:0:24:1.00:1.00:0.23:0.37), (Tuberculosis.gastrointestinal:7:4854:0:24:1.00:1.00:0.23:0.37), (Ad enosquamous.cell.lung.cancer.stage.IV:5:4856:0:24:1.00:1.00:0.17:0.29), (CD4.lymphocytes.dec reased:5:4856:0:24:1.00:1.00:0.17:0.29), (Haemangioma-thrombocytopenia.syndrome:5:4856:0:24:1.00:1.00:0.17:0.29), (Intra-uterine.contraceptive.device.insertion:5:4856:0:24:1.00:1.00:0.17:0.29), (Low.cardiac.output.syn drome:5:4856:0:24:1.00:1.00:0.17:0.29), (Anal.cancer.metastatic:4:4857:0:24:1.00:1.00:0.14:0.2 5), (Follicle.centre.lymphoma,.follicular.grade.I,.II,.III.stage.IV:4:4857:0:24:1.00:1.00:0.14:0.25), (Lip.oedema:4:4857:0:24:1.00:1.00:0.14:0.25), (Oestrogen.receptor.assay.negative:4:4857:0:24: 1.00:1.00:0.14:0.25), (Insulin-like.growth.factor:3:4858:0:24:1.00:1.00:0.11:0.20), (Simplex.virus.test.positive:3:4858:0:24:1.0 0:1.00:0.11:0.20), (Splenic.abscess:3:4858:0:24:1.00:1.00:0.11:0.20), (Steroid.activity:3:4858:0:2 4:1.00:1.00:0.11:0.20), (Tongue.cancer.metastatic:3:4858:0:24:1.00:1.00:0.11:0.20), (Acral.lentigi nous.melanoma:2:4859:0:24:1.00:1.00:0.08:0.14), (Biopsy.bone:2:4859:0:24:1.00:1.00:0.08:0.14), (Bipolar.disorder.relapse.prophylaxis:2:4859:0:24:1.00:1.00:0.08:0.14), (Bone.marrow.oedema: 2:4859:0:24:1.00:1.00:0.08:0.14), (Cardiac.output.decreased:2:4859:0:24:1.00:1.00:0.08:0.14), (C oronary.revascularisation:2:4859:0:24:1.00:1.00:0.08:0.14), (CSF.granulocyte.count.abnormal:2: 4859:0:24:1.00:1.00:0.08:0.14), (Genital.lesion:2:4859:0:24:1.00:1.00:0.08:0.14), (Lip.erosion:2:4 859:0:24:1.00:1.00:0.08:0.14), (Locked-in.syndrome:2:4859:0:24:1.00:1.00:0.08:0.14), (Malignant.neoplasm.of.pleura.metastatic:2:4859: 0:24:1.00:1.00:0.08:0.14), (Prostatic.haemorrhage:2:4859:0:24:1.00:1.00:0.08:0.14), (Thrombosis. mesenteric.vessel:2:4859:0:24:1.00:1.00:0.08:0.14), (Trans-sexualism:2:4859:0:24:1.00:1.00:0.08:0.14), (Atonic.urinary.bladder:1:4860:0:24:1.00:1.00:0.04: 0.08), (Coagulation.test.abnormal:1:4860:0:24:1.00:1.00:0.04:0.08), (Dissociative.amnesia:1:4860: 0:24:1.00:1.00:0.04:0.08), (Erythema.induratum:1:4860:0:24:1.00:1.00:0.04:0.08), (Hypnagogic. hallucination:1:4860:0:24:1.00:1.00:0.04:0.08), (Immunodeficiency.congenital:1:4860:0:24:1.00: 1.00:0.04:0.08), (Ischaemic.ulcer:1:4860:0:24:1.00:1.00:0.04:0.08), (Libido.increased:1:4860:0:24: 1.00:1.00:0.04:0.08), (Neuropsychiatric.syndrome:1:4860:0:24:1.00:1.00:0.04:0.08), (Nuclear.ma gnetic.resonance.imaging.liver:1:4860:0:24:1.00:1.00:0.04:0.08), (Pancreatitis.haemorrhagic:1:48 60:0:24:1.00:1.00:0.04:0.08), (Pleural.adhesion:1:4860:0:24:1.00:1.00:0.04:0.08), (Pleural.neopla sm:1:4860:0:24:1.00:1.00:0.04:0.08), (Spinal.ligament.ossification:1:4860:0:24:1.00:1.00:0.04:0. 08), (Taeniasis:1:4860:0:24:1.00:1.00:0.04:0.08), (Tongue.biting:1:4860:0:24:1.00:1.00:0.04:0.08), (Truncus.arteriosus.persistent:1:4860:0:24:1.00:1.00:0.04:0.08), (Uterine.infection:1:4860:0:24: 1.00:1.00:0.04:0.08), (Catabolic.state:0:4860:0:25:0.99:#NUM!:0.00:#NUM!), (Cerebral.malaria: 0:4860:0:25:0.99:#NUM!:0.00:#NUM!), (Cholesteatoma:0:4860:0:25:0.99:#NUM!:0.00:#NUM!), (Crush.injury:0:4860:0:25:0.99:#NUM!:0.00:#NUM!), (Diabetic.gangrene:0:4860:0:25:0.99:#N UM!:0.00:#NUM!), (Electrocardiogram:0:4860:0:25:0.99:#NUM!:0.00:#NUM!), (Environmental. exposure:0:4860:0:25:0.99:#NUM!:0.00:#NUM!), (Hypoglycaemic.unconsciousness:0:4860:0:25: 0.99:#NUM!:0.00:#NUM!), (Malignant.splenic.neoplasm:0:4860:0:25:0.99:#NUM!:0.00:#NUM !), (Meningeal.disorder:0:4860:0:25:0.99:#NUM!:0.00:#NUM!), (Muscle.operation:0:4860:0:25:0 0.99:#NUM!:0.00:#NUM!), (Necrotising.colitis:0:4860:0:25:0.99:#NUM!:0.00:#NUM!), (Oedema.mouth:0:4860:0:25:0.99:#NUM!:0.00:#NUM!), (Procedural.site.reaction:0:4860:0:25:0.99:#NU M!:0.00:#NUM!), (Regional.chemotherapy:0:4860:0:25:0.99:#NUM!:0.00:#NUM!), (Ventricular. remodelling:0:4860:0:25:0.99:#NUM!:0.00:#NUM!), (B-cell.lymphoma.stage.II:14:4846:0:25:0.99:1.00:0.36:0.53), (Teratoma:14:4846:0:25:0.99:1.00:0.3 6:0.53), (Acid.fast.bacilli.infection:10:4850:0:25:0.99:1.00:0.29:0.44), (Coagulation.factor.VIII.le vel:9:4851:0:25:0.99:1.00:0.26:0.42), (Infection.in.an.immunocompromised.host:8:4852:0:25:0.9 9:1.00:0.24:0.39), (Glomerular.filtration.rate.decreased:7:4853:0:25:0.99:1.00:0.22:0.36), (Anti-thrombin.antibody:6:4854:0:25:0.99:1.00:0.19:0.32), (Leiomyosarcoma.recurrent:6:4854:0:25:0. 99:1.00:0.19:0.32), (Pelvic.neoplasm:6:4854:0:25:0.99:1.00:0.19:0.32), (Peritoneal.mesothelioma. malignant:6:4854:0:25:0.99:1.00:0.19:0.32), (Transposition.of.the.great.vessels:6:4854:0:25:0.99: 1.00:0.19:0.32), (Viral.rash:6:4854:0:25:0.99:1.00:0.19:0.32), (Clinically.isolated.syndrome:5:485 5:0:25:0.99:1.00:0.17:0.29), (Nodular.melanoma:5:4855:0:25:0.99:1.00:0.17:0.29), (Optic.glioma: 5:4855:0:25:0.99:1.00:0.17:0.29), (Tolosa-Hunt.syndrome:5:4855:0:25:0.99:1.00:0.17:0.29), (Gastric.cancer.stage.III:4:4856:0:25:0.99:1.00: 0.14:0.24), (Neurosurgery:4:4856:0:25:0.99:1.00:0.14:0.24), (Preventive.surgery:4:4856:0:25:0.9 9:1.00:0.14:0.24), (Raoultella.omithinolytica.infection:4:4856:0:25:0.99:1.00:0.14:0.24), (Rett's. disorder:4:4856:0:25:0.99:1.00:0.14:0.24), (Vascular.headache:4:4856:0:25:0.99:1.00:0.14:0.24), (B orderline.ovarian.tumour:3:4857:0:25:0.99:1.00:0.11:0.19), (Change.of.bowel.habit:3:4857:0:25: 0.99:1.00:0.11:0.19), (Craniopharyngioma:3:4857:0:25:0.99:1.00:0.11:0.19), (Factor.XIII.deficien cy:3:4857:0:25:0.99:1.00:0.11:0.19), (Heart.rate.normal:3:4857:0:25:0.99:1.00:0.11:0.19), (Hypo pharyngeal.cancer.stage.IV:3:4857:0:25:0.99:1.00:0.11:0.19), (Inguinal.hemia.repair:3:4857:0:25: 0.99:1.00:0.11:0.19), (Painful.erection:3:4857:0:25:0.99:1.00:0.11:0.19), (Paradoxical.pressor.res ponse:3:4857:0:25:0.99:1.00:0.11:0.19), (Peripheral.nerve.neurostimulation:3:4857:0:25:0.99:1.0 0:0.11:0.19), (Punctate.keratitis:3:4857:0:25:0.99:1.00:0.11:0.19), (Renal.stone.removal:3:4857:0: 25:0.99:1.00:0.11:0.19), (Sexually.active:3:4857:0:25:0.99:1.00:0.11:0.19), (Tracheal.stenosis:3:4 857:0:25:0.99:1.00:0.11:0.19), (Transient.hypothyroxinaemia.of.prematurity:3:4857:0:25:0.99:1. 00:0.11:0.19), (Urinary.tract.neoplasm:3:4857:0:25:0.99:1.00:0.11:0.19), (Uterine.prolapse:3:485 7:0:25:0.99:1.00:0.11:0.19), (Abscess.oral:2:4858:0:25:0.99:1.00:0.07:0.14), (Aortic.occlusion:2: 4858:0:25:0.99:1.00:0.07:0.14), (Bile.output:2:4858:0:25:0.99:1.00:0.07:0.14), (Blastocystis.infec tion:2:4858:0:25:0.99:1.00:0.07:0.14), (Blood.insulin.increased:2:4858:0:25:0.99:1.00:0.07:0.14), (Cardiac.electrophysiologic.study:2:4858:0:25:0.99:1.00:0.07:0.14), (Computerised.tomogram.liv er:2:4858:0:25:0.99:1.00:0.07:0.14), (Contracted.bladder:2:4858:0:25:0.99:1.00:0.07:0.14), (Dysp hemia:2:4858:0:25:0.99:1.00:0.07:0.14), (Hyperandrogenism:2:4858:0:25:0.99:1.00:0.07:0.14), (Neural.tube.defect:2:4858:0:25:0.99:1.00:0.07:0.14), (Obstetrical.pulmonary.embolism:2:4858:0: 25:0.99:1.00:0.07:0.14), (Parathyroid.gland.operation:2:4858:0:25:0.99:1.00:0.07:0.14), (Peptic.ul cer.haemorrhage:2:4858:0:25:0.99:1.00:0.07:0.14), (Performance.status.decreased:2:4858:0:25:0. 99:1.00:0.07:0.14), (Pityriasis.rosea:2:4858:0:25:0.99:1.00:0.07:0.14), (Pneumococcal.bacteraemi a:2:4858:0:25:0.99:1.00:0.07:0.14), (Premature.ageing:2:4858:0:25:0.99:1.00:0.07:0.14), (Purulen ce:2:4858:0:25:0.99:1.00:0.07:0.14), (Ubiquinone:2:4858:0:25:0.99:1.00:0.07:0.14), (Abdominal. wall.disorder:1:4859:0:25:0.99:1.00:0.04:0.07), (Aerophagia:1:4859:0:25:0.99:1.00:0.04:0.07), (A pplication.site.acne:1:4859:0:25:0.99:1.00:0.04:0.07), (Artificial.heart.device.user:1:4859:0:25:0. 99:1.00:0.04:0.07), (Bronchial.irritation:1:4859:0:25:0.99:1.00:0.04:0.07), (Dyslexia:1:4859:0:25: 0.99:1.00:0.04:0.07), (Electric.shock:1:4859:0:25:0.99:1.00:0.04:0.07), (Encopresis:1:4859:0:25:0 0.99:1.00:0.04:0.07), (Fractured.coccyx:1:4859:0:25:0.99:1.00:0.04:0.07), (Hyper.IgE.syndrome:1: 4859:0:25:0.99:1.00:0.04:0.07), (Hyperalbuminaemia:1:4859:0:25:0.99:1.00:0.04:0.07), (Intestina 1.villi.atrophy:1:4859:0:25:0.99:1.00:0.04:0.07), (Liver.operation:1:4859:0:25:0.99:1.00:0.04:0.0 7), (Monoplegia:1:4859:0:25:0.99:1.00:0.04:0.07), (Oesophageal.carcinoma.recurrent:1:4859:0:25: 0.99:1.00:0.04:0.07), (Patella.fracture:1:4859:0:25:0.99:1.00:0.04:0.07), (Rash.vesicular:1:4859:0: 25:0.99:1.00:0.04:0.07), (Thyroid.function.test.normal:1:4859:0:25:0.99:1.00:0.04:0.07), (Toe.op eration:1:4859:0:25:0.99:1.00:0.04:0.07), (Upper.motor.neurone.lesion:1:4859:0:25:0.99:1.00:0.0 4:0.07), (Vascular.fragility:1:4859:0:25:0.99:1.00:0.04:0.07), (Acquired.complement.deficiency.di sease:0:4859:0:26:0.99:#NUM!:0.00:#NUM!), (Aortic.valve.prolapse:0:4859:0:26:0.99:#NUM!: 0.00:#NUM!), (Carotid.artery. aneurysm:0:4859:0:26:0.99:#NUM!:0.00:#NUM!), (Cerebrospinal. fluid.leakage:0:4859:0:26:0.99:#NUM!:0.00:#NUM!), (Decreased.bronchial.secretion:0:4859:0:2 6:0.99:#NUM!:0.00:#NUM!), (Foetal.heart.rate.abnormal:0:4859:0:26:0.99:#NUM!:0.00:#NUM!), (Hypozincaemia:0:4859:0:26:0.99:#NUM!:0.00:#NUM!), (Intervertebral.disc.annular.tear:0:48 59:0:26:0.99:#NUM!:0.00:#NUM!), (Lymphangiogram:0:4859:0:26:0.99:#NUM!:0.00:#NUM!), (Muscle.abscess:0:4859:0:26:0.99:#NUM!:0.00:#NUM!), (Nail.bed.infection:0:4859:0:26:0.99:#NUM!:0.00:#NUM!), (Nasal.abscess:0:4859:0:26:0.99:#NUM!:0.00:#NUM!), (Panic.disorder.wit h.agoraphobia:0:4859:0:26:0.99:#NUM!:0.00:#NUM!), (Paresis:0:4859:0:26:0.99:#NUM!:0.00:#NUM!), (Pneumococcal.sepsis:0:4859:0:26:0.99:#NUM!:0.00:#NUM!), (Somatic.dysfunction:0:4 859:0:26:0.99:#NUM!:0.00:#NUM!), (Superficial.siderosis.of.central.nervous.system:0:4859:0:2 6:0.99:#NUM!:0.00:#NUM!), (Thyroid.nodule.removal:0:4859:0:26:0.99:#NUM!:0.00:#NUM!), (Traumatic.amputation:0:4859:0:26:0.99:#NUM!:0.00:#NUM!), (T-cell.lymphoma.stage.IV:18:4841:0:26:0.99:1.00:0.41:0.58), (Generalised.non-convulsive.epilepsy:15:4844:0:26:0.99:1.00:0.37:0.54), (Acute.graft.versus.host.disease.in.liver:1 3:4846:0:26:0.99:1.00:0.33:0.50), (Parasitic.gastroenteritis:11:4848:0:26:0.99:1.00:0.30:0.46), (Al ternaria.infection:9:4850:0:26:0.99:1.00:0.26:0.41), (Henoch-Schonlein.purpura.nephritis:9:4850:0:26:0.99:1.00:0.26:0.41), (Necrotising.myositis:9:4850:0:26: 0.99:1.00:0.26:0.41), (Renal.tubular.disorder:9:4850:0:26:0.99:1.00:0.26:0.41), (Hepatitis.D:8:485 1:0:26:0.99:1.00:0.24:0.38), (Heteroplasia:8:4851:0:26:0.99:1.00:0.24:0.38), (Anti-cyclic.citrullinated.peptide.antibody.positive:7:4852:0:26:0.99:1.00:0.21:0.35), (Chronic.lymphoc ytic.leukaemia.transformation:7:4852:0:26:0.99:1.00:0.21:0.35), (Rhodococcus.infection:7:4852: 0:26:0.99:1.00:0.21:0.35), (Blood.creatine.increased:6:4853:0:26:0.99:1.00:0.19:0.32), (Infective. uveitis:6:4853:0:26:0.99:1.00:0.19:0.32), (Antithrombin.III:5:4854:0:26:0.99:1.00:0.16:0.28), (Ca mpylobacter.gastroenteritis:5:4854:0:26:0.99:1.00:0.16:0.28), (HIV.associated.nephropathy:5:48 54:0:26:0.99:1.00:0.16:0.28), (Sarcomatoid.mesothelioma:5:4854:0:26:0.99:1.00:0.16:0.28), (Acu te.lung.injury:4:4855:0:26:0.99:1.00:0.13:0.24), (Anal.inflammation:4:4855:0:26:0.99:1.00:0.13: 0.24), (Blister.infected:4:4855:0:26:0.99:1.00:0.13:0.24), (Bruton's.agammaglobulinaemia:4:4855: 0:26:0.99:1.00:0.13:0.24), (Liver.sarcoidosis:4:4855:0:26:0.99:1.00:0.13:0.24), (Synovial.disorde r:4:4855:0:26:0.99:1.00:0.13:0.24), (Transient.global.amnesia:4:4855:0:26:0.99:1.00:0.13:0.24), (Abnormal.weight.gain:3:4856:0:26:0.99:1.00:0.10:0.19), (Acetabulum.fracture:3:4856:0:26:0.99: 1.00:0.10:0.19), (Benign.pancreatic.neoplasm:3:4856:0:26:0.99:1.00:0.10:0.19), (Cerebral.small.v essel.ischaemic.disease:3:4856:0:26:0.99:1.00:0.10:0.19), (Exfoliative.rash:3:4856:0:26:0.99:1.0 0:0.10:0.19), (Hypercapnia:3:4856:0:26:0.99:1.00:0.10:0.19), (Hypersexuality:3:4856:0:26:0.99:1 0.00:0.10:0.19), (Multiple.organ.transplant.rejection:3:4856:0:26:0.99:1.00:0.10:0.19), (Pharyngeal.ulceration:3:4856:0:26:0.99:1.00:0.10:0.19), (Pleomorphic.malignant.fibrous.histiocytoma:3:485 6:0:26:0.99:1.00:0.10:0.19), (Polymenorrhoea:3:4856:0:26:0.99:1.00:0.10:0.19), (Pseudolympho ma:3:4856:0:26:0.99:1.00:0.10:0.19), (Arteriogram.carotid:2:4857:0:26:0.99:1.00:0.07:0.13), (Blo od.folate:2:4857:0:26:0.99:1.00:0.07:0.13), (Crystal.arthropathy:2:4857:0:26:0.99:1.00:0.07:0.13), (Diathermy:2:4857:0:26:0.99:1.00:0.07:0.13), (DNA.antibody.positive:2:4857:0:26:0.99:1.00:0. 07:0.13), (Exeresis:2:4857:0:26:0.99:1.00:0.07:0.13), (Haemorrhoids.thrombosed:2:4857:0:26:0.9 9:1.00:0.07:0.13), (HELLP.syndrome:2:4857:0:26:0.99:1.00:0.07:0.13), (Herpes.zoster.meningitis: 2:4857:0:26:0.99:1.00:0.07:0.13), (Metastases.to.thyroid:2:4857:0:26:0.99:1.00:0.07:0.13), (Myo pia:2:4857:0:26:0.99:1.00:0.07:0.13), (Occupational.exposure.to.noise:2:4857:0:26:0.99:1.00:0.0 7:0.13), (Perfume.sensitivity:2:4857:0:26:0.99:1.00:0.07:0.13), (Precursor.T-lymphoblastic.lymphoma/leukaemia.refractory:2:4857:0:26:0.99:1.00:0.07:0.13), (Squamous.cell.carcinoma.of.the.vulva:2:4857:0:26:0.99:1.00:0.07:0.13), (Trousseau's.syndrome:2:4857:0:26:0. 99:1.00:0.07:0.13), (Urine.albumin/creatinine.ratio:2:4857:0:26:0.99:1.00:0.07:0.13), (Wrong.pati ent.received.medication:2:4857:0:26:0.99:1.00:0.07:0.13), (Blood.pressure.normal:1:4858:0:26:0. 99:1.00:0.04:0.07), (Clostridium.difficile.immunisation:1:4858:0:26:0.99:1.00:0.04:0.07), (Coma. hepatic:1:4858:0:26:0.99:1.00:0.04:0.07), (Facial.paresis:1:4858:0:26:0.99:1.00:0.04:0.07), (Gastr ointestinal.sounds.abnormal:1:4858:0:26:0.99:1.00:0.04:0.07), (Hyperprothrombinaemia:1:4858: 0:26:0.99:1.00:0.04:0.07), (Injection.site.anaesthesia:1:4858:0:26:0.99:1.00:0.04:0.07), (Penile.ery thema:1:4858:0:26:0.99:1.00:0.04:0.07), (Pleomorphic.liposarcoma:1:4858:0:26:0.99:1.00:0.04:0 0.07), (Pneumatosis:1:4858:0:26:0.99:1.00:0.04:0.07), (Product.substitution.issue:1:4858:0:26:0.99: 1.00:0.04:0.07), (Ventricular.failure:1:4858:0:26:0.99:1.00:0.04:0.07), (Anti-platelet.antibody:0:4858:0:27:0.99:#NUM!:0.00:#NUM!), (Chronic.myelomonocytic.leukaemia.(in.remission):0:4858:0:27:0.99:#NUM!:0.00:#NUM!), (Colonic.abscess:0:4858:0:27:0.99:#NUM !:0.00:#NUM!), (Double.heterozygous.sickling.disorders:0:4858:0:27:0.99:#NUM!:0.00:#NUM!), (Epidural.test.dose:0:4858:0:27:0.99:#NUM!:0.00:#NUM!), (Hereditary.spastic.paraplegia:0:485 8:0:27:0.99:#NUM!:0.00:#NUM!), (Hypo.HDL.cholesterolaemia:0:4858:0:27:0.99:#NUM!:0.00: #NUM!), (Multiple.endocrine.neoplasia.Type.1:0:4858:0:27:0.99:#NUM!:0.00:#NUM!), (Myelo ma.cast.nephropathy:0:4858:0:27:0.99:#NUM!:0.00:#NUM!), (Myofascial.spasm:0:4858:0:27:0. 99:#NUM!:0.00:#NUM!), (Peritonsillar.abscess:0:4858:0:27:0.99:#NUM!:0.00:#NUM!), (Pigme ntary.glaucoma:0:4858:0:27:0.99:#NUM!:0.00:#NUM!), (Plasmacytosis:0:4858:0:27:0.99:#NU M!:0.00:#NUM!), (Progressive.familial.intrahepatic.cholestasis:0:4858:0:27:0.99:#NUM!:0.00:#NUM!), (Somatostatin.receptor.scan:0:4858:0:27:0.99:#NUM!:0.00:#NUM!), (Spinal.claudicatio n:0:4858:0:27:0.99:#NUM!:0.00:#NUM!), (Thrombin.time.prolonged:0:4858:0:27:0.99:#NUM!: 0.00:#NUM!), (Ventricular.tachyarrhythmia:0:4858:0:27:0.99:#NUM!:0.00:#NUM!), (Fibromato sis:13:4845:0:27:0.99:1.00:0.33:0.49), (Precursor.B-lymphoblastic.lymphoma.refractory:12:4846:0:27:0.99:1.00:0.31:0.47), (Cogan's.syndrome:10:48 48:0:27:0.99:1.00:0.27:0.43), (Adenocarcinoma.of.appendix:8:4850:0:27:0.99:1.00:0.23:0.37), (F actor.II.deficiency:8:4850:0:27:0.99:1.00:0.23:0.37), (High.risk.pregnancy:8:4850:0:27:0.99:1.00: 0.23:0.37), (Cerebral.microangiopathy:7:4851:0:27:0.99:1.00:0.21:0.34), (Delivery:7:4851:0:27:0 0.99:1.00:0.21:0.34), (HIV.carrier:7:4851:1:26:0.99:0.88:0.21:0.34), (Lactic.acidosis:7:4851:0:27:0 0.99:1.00:0.21:0.34), (Anal.haemorrhage:5:4853:0:27:0.99:1.00:0.16:0.27), (Neuroendocrine.tumo ur.of.the.lung.metastatic:5:4853:0:27:0.99:1.00:0.16:0.27), (Anaemia.of.pregnancy:4:4854:0:27:0 0.99:1.00:0.13:0.23), (Carotid.artery.dissection:4:4854:0:27:0.99:1.00:0.13:0.23), (Gastric.antral.va scular.ectasia:4:4854:0:27:0.99:1.00:0.13:0.23), (Gastric.lavage.abnormal:4:4854:0:27:0.99:1.00: 0.13:0.23), (Inguinal.hernia:4:4854:0:27:0.99:1.00:0.13:0.23), (Iris.adhesions:4:4854:0:27:0.99:1. 00:0.13:0.23), (Nitrogen.balance.negative:4:4854:0:27:0.99:1.00:0.13:0.23), (Oral.bacterial.infecti on:4:4854:0:27:0.99:1.00:0.13:0.23), (Pharyngitis.bacterial:4:4854:0:27:0.99:1.00:0.13:0.23), (Ret inal.vascular.thrombosis:4:4854:0:27:0.99:1.00:0.13:0.23), (Tongue.blistering:4:4854:0:27:0.99:1 0.00:0.13:0.23), (Tubo-ovarian.abscess:4:4854:0:27:0.99:1.00:0.13:0.23), (Vaginal.cancer:4:4854:0:27:0.99:1.00:0.13:0. 23), (Cardiac.cirrhosis:3:4855:0:27:0.99:1.00:0.10:0.18), (Cognitive.rehabilitation:3:4855:0:27:0. 99:1.00:0.10:0.18), (Erythema.of.eyelid:3:4855:0:27:0.99:1.00:0.10:0.18), (Gingival.erythema:3:4 855:0:27:0.99:1.00:0.10:0.18), (Increased.tendency.to.bruise:3:4855:0:27:0.99:1.00:0.10:0.18), (P ost.procedural.oedema:3:4855:0:27:0.99:1.00:0.10:0.18), (Pseudarthrosis:3:4855:0:27:0.99:1.00:0 0.10:0.18), (Scrotal.swelling:3:4855:0:27:0.99:1.00:0.10:0.18), (10:0.18), (Acanthosis.nigricans:2:4856:0:27:0.99:1.00:0.07:0.13), (Bladder.neck.obstruction:2:48 56:0:27:0.99:1.00:0.07:0.13), (Bladder.transitional.cell.carcinoma.stage.0:2:4856:0:27:0.99:1.00: 0.07:0.13), (Coronary.artery.aneurysm:2:4856:0:27:0.99:1.00:0.07:0.13), (Cytoreductive.surgery: 2:4856:0:27:0.99:1.00:0.07:0.13), (Decreased.interest:2:4856:0:27:0.99:1.00:0.07:0.13), (Freezing.phenomenon:2:4856:0:27:0.99:1.00:0.07:0.13), (Hormone.refractory.breast.cancer:2:4856:0:27:0 0.99:1.00:0.07:0.13), (Polypectomy:2:4856:0:27:0.99:1.00:0.07:0.13), (Prophylaxis.against.Rh.isoi mmunisation:2:4856:0:27:0.99:1.00:0.07:0.13), (Prosthesis.implantation:2:4856:0:27:0.99:1.00:0. 07:0.13), (Reading.disorder:2:4856:0:27:0.99:1.00:0.07:0.13), (Right.ventricular.systolic.pressure. increased:2:4856:0:27:0.99:1.00:0.07:0.13), (Toxic.encephalopathy:2:4856:0:27:0.99:1.00:0.07:0 0.13), (ACTH.stimulation.test:1:4857:0:27:0.99:1.00:0.04:0.07), (Alpha-1.anti-trypsin.abnormal:1:4857:0:27:0.99:1.00:0.04:0.07), (Anticholinergic.syndrome:1:4857:0:27:0.99: 1.00:0.04:0.07), (Anxiety.disorder.due.to.a.general.medical.condition:1:4857:0:27:0.99:1.00:0.04: 0.07), (Biliary.drainage:1:4857:0:27:0.99:1.00:0.04:0.07), (Biopsy.brain:1:4857:0:27:0.99:1.00:0. 04:0.07), (Carpal.tunnel.decompression:1:4857:0:27:0.99:1.00:0.04:0.07), (Cerebral.vasoconstrict ion:1:4857:0:27:0.99:1.00:0.04:0.07), (Chemical.poisoning:1:4857:0:27:0.99:1.00:0.04:0.07), (Inf ective.aneurysm:1:4857:0:27:0.99:1.00:0.04:0.07), (Medical.device.removal:1:4857:0:27:0.99:1. 00:0.04:0.07), (Metastases.to.biliary.tract:1:4857:0:27:0.99:1.00:0.04:0.07), (Middle.lobe.syndro me:1:4857:0:27:0.99:1.00:0.04:0.07), (Monoclonal.antibody.chemoimmunoconjugate.therapy:1:4 857:0:27:0.99:1.00:0.04:0.07), (Prostatic.pain:1:4857:0:27:0.99:1.00:0.04:0.07), (Prothrombin.lev el.abnormal:1:4857:0:27:0.99:1.00:0.04:0.07), (Rectosigmoid.cancer.recurrent:1:4857:0:27:0.99: 1.00:0.04:0.07), (Shoulder.deformity:1:4857:0:27:0.99:1.00:0.04:0.07), (Tachycardia.foetal:1:485 7:0:27:0.99:1.00:0.04:0.07), (Umbilical.heria.repair:1:4857:0:27:0.99:1.00:0.04:0.07), (Vascular. stent.insertion:1:4857:0:27:0.99:1.00:0.04:0.07), (Visceral.pain:1:4857:0:27:0.99:1.00:0.04:0.07), (Vitamin.B2.deficiency:1:4857:0:27:0.99:1.00:0.04:0.07), (Actinomycotic.pulmonary.infection:0: 4857:0:28:0.99:#NUM!:0.00:#NUM!), (Bacterial.diarrhoea:0:4857:0:28:0.99:#NUM!:0.00:#NU M!), (Biliary.tract.infection:0:4857:0:28:0.99:#NUM!:0.00:#NUM!), (Blood.pressure.diastolic:0:4 857:0:28:0.99:#NUM!:0.00:#NUM!), (Breath.sounds.abnormal:0:4857:0:28:0.99:#NUM!:0.00:#NUM!), (Hypokinesia:0:4857:0:28:0.99:#NUM!:0.00:#NUM!), (Intrusive.thoughts:0:4857:0:28:0 0.99:#NUM!:0.00:#NUM!), (Jaw.fracture:0:4857:0:28:0.99:#NUM!:0.00:#NUM!), (No.therapeutic.response:0:4857:0:28:0.99:#NUM!:0.00:#NUM!), (Prostatic.hypoplasia:0:4857:0:28:0.99:#NU M!:0.00:#NUM!), (Soft.tissue.inflammation:0:4857:0:28:0.99:#NUM!:0.00:#NUM!), (Subacute.e ndocarditis:0:4857:0:28:0.99:#NUM!:0.00:#NUM!), (Tonsillitis.bacterial:0:4857:0:28:0.99:#NU M!:0.00:#NUM!), (Vaginal.odour:0:4857:0:28:0.99:#NUM!:0.00:#NUM!), (Vascular.shunt:0:485 7:0:28:0.99:#NUM!:0.00:#NUM!), (Vertebral.artery.dissection:0:4857:0:28:0.99:#NUM!:0.00:#NUM!), (Vulvovaginal.erythema:0:4857:0:28:0.99:#NUM!:0.00:#NUM!), (Hodgkin's.disease.sta ge.IL:13:4844:0:28:0.99:1.00:0.32:0.48), (HIV.antibody.positive:11:4846:0:28:0.99:1.00:0.28:0.4 4), (Rheumatoid.nodule:11:4846:0:28:0.99:1.00:0.28:0.44), (Atypical.mycobacterium.test.positive: 10:4847:1:27:0.99:0.91:0.27:0.42), (Mononeuropathy.multiplex:10:4847:0:28:0.99:1.00:0.26:0.4 2), (Optic.neuropathy:10:4847:0:28:0.99:1.00:0.26:0.42), (Human.epidermal.growth.factor.recept or.negative:9:4848:0:28:0.99:1.00:0.24:0.39), (Biliary.cancer.metastatic:8:4849:0:28:0.99:1.00:0. 22:0.36), (Ovarian.granulosa.cell.tumour:8:4849:0:28:0.99:1.00:0.22:0.36), (Transverse.sinus.thro mbosis:8:4849:0:28:0.99:1.00:0.22:0.36), (Small.intestine.transplant:7:4850:0:28:0.99:1.00:0.20: 0.33), (Extravasation:6:4851:0:28:0.99:1.00:0.18:0.30), (Acquired.epidermolysis.bullosa:5:4852:0: 28:0.99:1.00:0.15:0.26), (Acute.hepatitis.B:5:4852:0:28:0.99:1.00:0.15:0.26), (Enterocutaneous.fi stula:5:4852:0:28:0.99:1.00:0.15:0.26), (Fallopian.tube.cancer.metastatic:5:4852:0:28:0.99:1.00:0 0.15:0.26), (Keratinising.squamous.cell.carcinoma.of.nasopharynx:5:4852:0:28:0.99:1.00:0.15:0.2 6), (Mesotherapy:5:4852:0:28:0.99:1.00:0.15:0.26), (Multi-organ.disorder:5:4852:0:28:0.99:1.00:0.15:0.26), (Pulmonary.vascular.resistance.abnormality:5:4 852:0:28:0.99:1.00:0.15:0.26), (Restrictive.cardiomyopathy:5:4852:0:28:0.99:1.00:0.15:0.26), (Ut erine.dilation.and.curettage:5:4852:0:28:0.99:1.00:0.15:0.26), (Aphonia:4:4853:0:28:0.99:1.00:0. 13:0.22), (Noninfective.sialoadenitis:4:4853:0:28:0.99:1.00:0.13:0.22), (Purine.metabolism.disord er:4:4853:0:28:0.99:1.00:0.13:0.22), (Blood.thyroid.stimulating.hormone.abnormal:3:4854:0:28:0 0.99:1.00:0.10:0.18), (Cholinesterase.inhibition:3:4854:0:28:0.99:1.00:0.10:0.18), (Dyspnoea.parox ysmal.noctumal:3:4854:0:28:0.99:1.00:0.10:0.18), (Liddle's.syndrome:3:4854:0:28:0.99:1.00:0.1 0:0.18), (Photopheresis:3:4854:0:28:0.99:1.00:0.10:0.18), (Relapsing.fever:3:4854:0:28:0.99:1.00: 0.10:0.18), (Senile.ankylosing.vertebral.hyperostosis:3:4854:0:28:0.99:1.00:0.10:0.18), (Vaccinati on.complication:3:4854:0:28:0.99:1.00:0.10:0.18), (Arterial.therapeutic.procedure:2:4855:0:28:0. 99:1.00:0.07:0.13), (Denture.wearer:2:4855:0:28:0.99:1.00:0.07:0.13), (Family.stress:2:4855:0:28: 0.99:1.00:0.07:0.13), (Gastrointestinal.wall.thickening:2:4855:0:28:0.99:1.00:0.07:0.13), (Hyperp hosphatasaemia:2:4855:0:28:0.99:1.00:0.07:0.13), (Insulin-like.growth.factor.decreased:2:4855:0:28:0.99:1.00:0.07:0.13), (Lipoma:2:4855:0:28:0.99:1.00:0. 07:0.13), (Nasal.discharge.discolouration:2:4855:0:28:0.99:1.00:0.07:0.13), (Photodermatosis:2:4 855:0:28:0.99:1.00:0.07:0.13), (Spinal.X-ray:2:4855:0:28:0.99:1.00:0.07:0.13), (Upper.respiratory.fungal.infection:2:4855:0:28:0.99:1.00: 0.07:0.13), (Vertebral.artery.stenosis:2:4855:0:28:0.99:1.00:0.07:0.13), (Aneurysmal.bone.cyst:1: 4856:0:28:0.99:1.00:0.03:0.07), (Aortic.surgery:1:4856:0:28:0.99:1.00:0.03:0.07), (Blood.immun oglobulin.E:1:4856:0:28:0.99:1.00:0.03:0.07), (Cervical.cord.compression:1:4856:0:28:0.99:1.00: 0.03:0.07), (Chronic.leukaemia.in.remission:1:4856:0:28:0.99:1.00:0.03:0.07), (Fluid.balance.ass essment:1:4856:0:28:0.99:1.00:0.03:0.07), (Hypermetabolism:1:4856:0:28:0.99:1.00:0.03:0.07), (Lipoprotein.(a):1:4856:0:28:0.99:1.00:0.03:0.07), (Low.turnover.osteopathy:1:4856:0:28:0.99:1.0 0:0.03:0.07), (Malignant.melanoma.in.situ:1:4856:0:28:0.99:1.00:0.03:0.07), (Orbital.infection:1: 4856:0:28:0.99:1.00:0.03:0.07), (Purulent.discharge:1:4856:0:28:0.99:1.00:0.03:0.07), (Renal.mas s:1:4856:0:28:0.99:1.00:0.03:0.07), (Spinal.decompression:1:4856:0:28:0.99:1.00:0.03:0.07), (Sto ma.site.irritation:1:4856:0:28:0.99:1.00:0.03:0.07), (Venous.stenosis:1:4856:0:28:0.99:1.00:0.03: 0.07), (Carotid.artery.bypass:0:4856:0:29:0.99:#NUM!:0.00:#NUM!), (Hepatic.infection.fungal:0: 4856:0:29:0.99:#NUM!:0.00:#NUM!), (Hepatic.sequestration:0:4856:0:29:0.99:#NUM!:0.00:#N UM!), (Mallory-Weiss.syndrome:0:4856:0:29:0.99:#NUM!:0.00:#NUM!), (Mean.arterial.pressure.decreased:0:48 56:0:29:0.99:#NUM!:0.00:#NUM!), (Occult.blood.positive:0:4856:0:29:0.99:#NUM!:0.00:#NU M!), (Skin.depigmentation:0:4856:0:29:0.99:#NUM!:0.00:#NUM!), (Traumatic.intracranial.haem orrhage:0:4856:0:29:0.99:#NUM!:0.00:#NUM!), (Type.II.hyperlipidaemia:0:4856:0:29:0.99:#N UM!:0.00:#NUM!), (Urogram:0:4856:0:29:0.99:#NUM!:0.00:#NUM!), (Vitamin.K:0:4856:0:29: 0.99:#NUM!:0.00:#NUM!), (Water.intoxication:0:4856:0:29:0.99:#NUM!:0.00:#NUM!), (Intesti nal.adenocarcinoma:15:4841:0:29:0.99:1.00:0.34:0.51), (Factor.VIII.inhibition:13:4843:0:29:0.99: 1.00:0.31:0.47), (Ependymoma.malignant:11:4845:0:29:0.99:1.00:0.28:0.43), (Lymphoma.transf ormation:11:4845:0:29:0.99:1.00:0.28:0.43), (Ovulation.disorder:10:4846:2:27:0.99:0.83:0.27:0. 41), (Insulin-like.growth.factor.increased:10:4846:0:29:0.99:1.00:0.26:0.41), (Chronic.lymphocytic.leukaemia. stage.4:9:4847:0:29:0.99:1.00:0.24:0.38), (Antineutrophil.cytoplasmic.antibody:8:4848:0:29:0.99: 1.00:0.22:0.36), (Colon.cancer.stage.II:7:4849:0:29:0.99:1.00:0.19:0.33), (Blood.insulin.decrease d:6:4850:0:29:0.99:1.00:0.17:0.29), (Carcinoid.tumour.of.the.caecum:6:4850:0:29:0.99:1.00:0.17: 0.29), (Congenital.arterial.malformation:6:4850:0:29:0.99:1.00:0.17:0.29), (Miosis:6:4850:0:29:0 0.99:1.00:0.17:0.29), (Progesterone.receptor.assay.negative:6:4850:0:29:0.99:1.00:0.17:0.29), (Anti nuclear.antibody.positive:5:4851:0:29:0.99:1.00:0.15:0.26), (Neuroendocrine.tumour.of.the.lung: 5:4851:0:29:0.99:1.00:0.15:0.26), (Rhesus.antibodies.negative:5:4851:0:29:0.99:1.00:0.15:0.26), (Bradyphrenia:4:4852:0:29:0.99:1.00:0.12:0.22), (Congenital.hydrocephalus:4:4852:0:29:0.99:1.0 0:0.12:0.22), (Gaucher's.disease.type.III:4:4852:0:29:0.99:1.00:0.12:0.22), (Mixed.hepatocellular. cholangiocarcinoma:4:4852:0:29:0.99:1.00:0.12:0.22), (Ovarian.cancer.stage.II:4:4852:0:29:0.99: 1.00:0.12:0.22), (Prostatic.mass:4:4852:0:29:0.99:1.00:0.12:0.22), (Warm.type.haemolytic.anaemi a:4:4852:0:29:0.99:1.00:0.12:0.22), (Antipsychotic.drug.level.therapeutic:3:4853:0:29:0.99:1.00: 0.09:0.17), (BRAF.V600E.mutation.positive:3:4853:0:29:0.99:1.00:0.09:0.17), (Carotid.artery.thr ombosis:3:4853:0:29:0.99:1.00:0.09:0.17), (Childhood.psychosis:3:4853:0:29:0.99:1.00:0.09:0.1 7), (Complications.of.bone.marrow.transplant:3:4853:0:29:0.99:1.00:0.09:0.17), (Defaecation.urg ency:3:4853:0:29:0.99:1.00:0.09:0.17), (Ex-drug.abuser:3:4853:0:29:0.99:1.00:0.09:0.17), (Gastric.pH:3:4853:0:29:0.99:1.00:0.09:0.17), (HE R2.gene.amplification:3:4853:0:29:0.99:1.00:0.09:0.17), (Iodine.allergy:3:4853:0:29:0.99:1.00:0. 09:0.17), (Ligament.pain:3:4853:0:29:0.99:1.00:0.09:0.17), (Multiple.congenital.abnormalities:3: 4853:0:29:0.99:1.00:0.09:0.17), (Neovascularisation:3:4853:0:29:0.99:1.00:0.09:0.17), (Osteoscle rosis:3:4853:0:29:0.99:1.00:0.09:0.17), (Post.thrombotic.syndrome:3:4853:0:29:0.99:1.00:0.09:0. 17), (Postrenal.failure:3:4853:0:29:0.99:1.00:0.09:0.17), (Sinusitis.00:0.09:0.17), (Accident.at.work:2:4854:0:29:0.99:1.00:0.06:0.12), (Disturbance.in.sexual.arousal: 2:4854:0:29:0.99:1.00:0.06:0.12), (Hypotrichosis:2:4854:0:29:0.99:1.00:0.06:0.12), (Intestinal.op eration:2:4854:0:29:0.99:1.00:0.06:0.12), (Keratitis.fungal:2:4854:0:29:0.99:1.00:0.06:0.12), (Lap aroscopy:2:4854:0:29:0.99:1.00:0.06:0.12), (Lip.infection:2:4854:0:29:0.99:1.00:0.06:0.12), (Oes ophageal.neoplasm:2:4854:0:29:0.99:1.00:0.06:0.12), (Orchidectomy:2:4854:0:29:0.99:1.00:0.06: 0.12), (Tetrahydrobiopterin.deficiency:2:4854:0:29:0.99:1.00:0.06:0.12), (Tinea.capitis:2:4854:0: 29:0.99:1.00:0.06:0.12), (Venipuncture:2:4854:0:29:0.99:1.00:0.06:0.12), (Vulvovaginal.injury:2: 4854:0:29:0.99:1.00:0.06:0.12), (Alcoholic.seizure:1:4855:0:29:0.99:1.00:0.03:0.06), (Androgens. abnormal:1:4855:0:29:0.99:1.00:0.03:0.06), (Blood.antidiuretic.hormone.decreased:1:4855:0:29: 0.99:1.00:0.03:0.06), (Breast.swelling:1:4855:0:29:0.99:1.00:0.03:0.06), (Coagulation.factor.defic iency:1:4855:0:29:0.99:1.00:0.03:0.06), (Conjunctivochalasis:1:4855:0:29:0.99:1.00:0.03:0.06), (Deafness.unilateral:1:4855:0:29:0.99:1.00:0.03:0.06), (Diet.failure:1:4855:0:29:0.99:1.00:0.03:0. 06), (Dissociation:1:4855:0:29:0.99:1.00:0.03:0.06), (Gastroenteritis.clostridial:1:4855:0:29:0.99: 1.00:0.03:0.06), (Glossopharyngeal.neuralgia:1:4855:0:29:0.99:1.00:0.03:0.06), (Haematoma.infe ction:1:4855:0:29:0.99:1.00:0.03:0.06), (Hospice.care:1:4855:0:29:0.99:1.00:0.03:0.06), (Infectio n.protozoal:1:4855:0:29:0.99:1.00:0.03:0.06), (Intra-abdominal.haemorrhage:1:4855:0:29:0.99:1.00:0.03:0.06), (Ketogenic.diet:1:4855:0:29:0.99:1.00: 0.03:0.06), (Massage:1:4855:0:29:0.99:1.00:0.03:0.06), (Mitochondrial.myopathy:1:4855:0:29:0. 99:1.00:0.03:0.06), (Nerve.root.injury:1:4855:0:29:0.99:1.00:0.03:0.06), (Pulmonary.function.test. abnormal:1:4855:0:29:0.99:1.00:0.03:0.06), (Tay-Sachs.disease:1:4855:0:29:0.99:1.00:0.03:0.06), (Tonsillar.hypertrophy:1:4855:0:29:0.99:1.00:0. 03:0.06), (Application.site.pain:0:4855:0:30:0.99:#NUM!:0.00:#NUM!), (Blood.homocysteine:0: 4855:0:30:0.99:#NUM!:0.00:#NUM!), (Burkholderia.cepacia.complex.infection:0:4855:0:30:0.9 9:#NUM!:0.00:#NUM!), (Fasting:0:4855:0:30:0.99:#NUM!:0.00:#NUM!), (Fibrin.decreased:0:48 55:0:30:0.99:#NUM!:0.00:#NUM!), (Gastrointestinal.telangiectasia:0:4855:0:30:0.99:#NUM!:0. 00:#NUM!), (Lipoatrophy:0:4855:0:30:0.99:#NUM!:0.00:#NUM!), (Pancreatitis.necrotising:0:48 55:0:30:0.99:#NUM!:0.00:#NUM!), (Post.procedural.bile.leak:0:4855:0:30:0.99:#NUM!:0.00:#N UM!), (Total.bile.acids.increased:0:4855:0:30:0.99:#NUM!:0.00:#NUM!), (Urinary.retention.post operative:0:4855:0:30:0.99:#NUM!:0.00:#NUM!), (Vaginal.abscess:0:4855:0:30:0.99:#NUM!:0. 00:#NUM!), (Hepatitis.E:12:4843:0:30:0.99:1.00:0.29:0.44), (Autoimmune.encephalopathy:10:48 45:0:30:0.99:1.00:0.25:0.40), (Mycobacterium.kansasii.infection:7:4848:0:30:0.99:1.00:0.19:0.32), (Pseudomyxoma.peritonei:7:4848:0:30:0.99:1.00:0.19:0.32), (Squamous.cell.carcinoma.of.phar ynx:6:4849:0:30:0.99:1.00:0.17:0.29), (Device.related.thrombosis:5:4850:0:30:0.99:1.00:0.14:0.2 5), (Ethmoid.sinus.surgery:5:4850:0:30:0.99:1.00:0.14:0.25), (External.ear.cellulitis:5:4850:0:30: 0.99:1.00:0.14:0.25), (Lymphocytic.hypophysitis:5:4850:0:30:0.99:1.00:0.14:0.25), (Malignant.an orectal.neoplasm:5:4850:0:30:0.99:1.00:0.14:0.25), (Open.reduction.of.fracture:5:4850:0:30:0.99: 1.00:0.14:0.25), (Cervical.incompetence:4:4851:0:30:0.99:1.00:0.12:0.21), (Gliosarcoma:4:4851: 0:30:0.99:1.00:0.12:0.21), (Lip.ulceration:4:4851:0:30:0.99:1.00:0.12:0.21), (Oligomenorrhoea:4: 4851:0:30:0.99:1.00:0.12:0.21), (Ovulation.pain:4:4851:0:30:0.99:1.00:0.12:0.21), (Spinocerebell ar.ataxia:4:4851:0:30:0.99:1.00:0.12:0.21), (Adnexa.uteri.pain:3:4852:0:30:0.99:1.00:0.09:0.17), (Amaurosis.fugax:3:4852:0:30:0.99:1.00:0.09:0.17), (Breast.cyst:3:4852:0:30:0.99:1.00:0.09:0.17), (Excessive.dietary.fibre.intake:3:4852:0:30:0.99:1.00:0.09:0.17), (Hypophysectomy:3:4852:0:30: 0.99:1.00:0.09:0.17), (Nail.ridging:3:4852:0:30:0.99:1.00:0.09:0.17), (Optic.ischaemic.neuropath y:3:4852:0:30:0.99:1.00:0.09:0.17), (Plasmodium.falciparum.infection:3:4852:0:30:0.99:1.00:0.0 9:0.17), (Unresponsive.to.stimuli:3:4852:0:30:0.99:1.00:0.09:0.17), (Urinary.bladder.polyp:3:485 2:0:30:0.99:1.00:0.09:0.17), (Alcohol.poisoning:2:4853:0:30:0.99:1.00:0.06:0.12), (Burkholderia. pseudomallei.infection:2:4853:0:30:0.99:1.00:0.06:0.12), (Candida.pneumonia:2:4853:0:30:0.99: 1.00:0.06:0.12), (Cardiac.neoplasm.malignant:2:4853:0:30:0.99:1.00:0.06:0.12), (Catheter.site.cel lulitis:2:4853:0:30:0.99:1.00:0.06:0.12), (Cerebral.hypoperfusion:2:4853:0:30:0.99:1.00:0.06:0.1 2), (Deficiency.of.bile.secretion:2:4853:0:30:0.99:1.00:0.06:0.12), (Diastolic.hypertension:2:4853: 0:30:0.99:1.00:0.06:0.12), (Fungal.sepsis:2:4853:0:30:0.99:1.00:0.06:0.12), (Joint.injection:2:485 3:0:30:0.99:1.00:0.06:0.12), (Laryngeal.disorder:2:4853:0:30:0.99:1.00:0.06:0.12), (Pericardial. m esothelioma.malignant:2:4853:0:30:0.99:1.00:0.06:0.12), (Pulmonary.artery.therapeutic.procedur e:2:4853:0:30:0.99:1.00:0.06:0.12), (Retinal.migraine:2:4853:0:30:0.99:1.00:0.06:0.12), (Thyroid. atrophy:2:4853:0:30:0.99:1.00:0.06:0.12), (Abdominal.mass:1:4854:0:30:0.99:1.00:0.03:0.06), (A cute.endocarditis:1:4854:0:30:0.99:1.00:0.03:0.06), (Alcoholic.psychosis:1:4854:0:30:0.99:1.00:0 0.03:0.06), (Cubital.tunnel.syndrome:1:4854:0:30:0.99:1.00:0.03:0.06), (Feeling.of.body.temperatu re.change:1:4854:0:30:0.99:1.00:0.03:0.06), (Gingival.abscess:1:4854:0:30:0.99:1.00:0.03:0.06), (Growth.hormone-producing.pituitary.tumour:1:4854:0:30:0.99:1.00:0.03:0.06), (Myocardial.necrosis.marker.increa sed:1:4854:0:30:0.99:1.00:0.03:0.06), (Oedema.mucosal:1:4854:0:30:0.99:1.00:0.03:0.06), (Postu ring:1:4854:0:30:0.99:1.00:0.03:0.06), (Prothrombin.level.decreased:1:4854:0:30:0.99:1.00:0.03: 0.06), (Pulse.pressure.increased:1:4854:0:30:0.99:1.00:0.03:0.06), (Skin.discomfort:1:4854:0:30:0 0.99:1.00:0.03:0.06), (Thyroid.pain:1:4854:0:30:0.99:1.00:0.03:0.06), (Acute.abdomen:0:4854:0:3 1:0.99:#NUM!:0.00:#NUM!), (Campylobacter.colitis:0:4854:0:31:0.99:#NUM!:0.00:#NUM!), (C erebellar.atrophy:0:4854:0:31:0.99:#NUM!:0.00:#NUM!), (Choreoathetosis:0:4854:0:31:0.99:#N UM!:0.00:#NUM!), (Corticobasal.degeneration:0:4854:0:31:0.99:#NUM!:0.00:#NUM!), (Renal.c ell.carcinoma.stage.III:0:4854:0:31:0.99:#NUM!:0.00:#NUM!), (Tumour. excision:0:4854:0:31:0. 99:#NUM!:0.00:#NUM!), (Viral.load:0:4854:0:31:0.99:#NUM!:0.00:#NUM!), (HIV.infection.C DC.category.C:21:4833:1:30:0.99:0.95:0.41:0.58), (Congenital.aplastic.anaemia:18:4836:0:31:0. 99:1.00:0.37:0.54), (Atonic.seizures:15:4839:1:30:0.99:0.94:0.33:0.49), (Hepatic.cancer.recurrent: 13:4841:0:31:0.99:1.00:0.30:0.46), (Eosinophilic.fasciitis:11:4843:0:31:0.99:1.00:0.26:0.42), (Sch istosomiasis:11:4843:0:31:0.99:1.00:0.26:0.42), (Hashimoto's.encephalopathy:10:4844:0:31:0.99: 1.00:0.24:0.39), (Histiocytic.sarcoma:10:4844:0:31:0.99:1.00:0.24:0.39), (Acquired.gene.mutatio n:8:4846:0:31:0.99:1.00:0.21:0.34), (Rectal.neoplasm:8:4846:0:31:0.99:1.00:0.21:0.34), (Hepatic. mass:7:4847:0:31:0.99:1.00:0.18:0.31), (Testicular.malignant.teratoma:7:4847:0:31:0.99:1.00:0.1 8:0.31), (Wagner's.disease:7:4847:0:31:0.99:1.00:0.18:0.31), (Granulocyte.count.decreased:6:484 8:0:31:0.99:1.00:0.16:0.28), (Ophthalmoplegia:6:4848:0:31:0.99:1.00:0.16:0.28), (Sarcopenia:6:4 848:0:31:0.99:1.00:0.16:0.28), (Cervix.carcinoma.stage.II:5:4849:0:31:0.99:1.00:0.14:0.24), (Div erticular.perforation:5:4849:0:31:0.99:1.00:0.14:0.24), (Gonadal.dysgenesis:5:4849:0:31:0.99:1.0 0:0.14:0.24), (Intracranial.germ.cell.tumour:5:4849:0:31:0.99:1.00:0.14:0.24), (Meningoencephali tis.viral:5:4849:0:31:0.99:1.00:0.14:0.24), (Peripheral.motor.neuropathy:5:4849:0:31:0.99:1.00:0. 14:0.24), (Anticoagulation.drug.level.below.therapeutic:4:4850:0:31:0.99:1.00:0.11:0.21), (Bladd er.cancer.stage.IV:4:4850:0:31:0.99:1.00:0.11:0.21), (Histamine.abnormal:4:4850:0:31:0.99:1.00: 0.11:0.21), (Lung.carcinoma.cell.type.unspecified.stage.II:4:4850:0:31:0.99:1.00:0.11:0.21), (Rect al.ulcer:4:4850:0:31:0.99:1.00:0.11:0.21), (Anticoagulation.drug.level.therapeutic:3:4851:0:31:0. 99:1.00:0.09:0.16), (Cervix.neoplasm:3:4851:0:31:0.99:1.00:0.09:0.16), (Child-Pugh-Turcotte.score.increased:3:4851:0:31:0.99:1.00:0.09:0.16), (Eyelid.injury:3:4851:0:31:0.99:1.00: 0.09:0.16), (Foreign.body.in.eye:3:4851:0:31:0.99:1.00:0.09:0.16), (Limb.crushing.injury:3:4851: 0:31:0.99:1.00:0.09:0.16), (Luteinising.hormone.deficiency:3:4851:0:31:0.99:1.00:0.09:0.16), (Ne urosyphilis:3:4851:0:31:0.99:1.00:0.09:0.16), (pH.urine.abnormal:3:4851:0:31:0.99:1.00:0.09:0.1 6), (Splenic.infarction:3:4851:0:31:0.99:1.00:0.09:0.16), (Uterine.hypotonus:3:4851:0:31:0.99:1.0 0:0.09:0.16), (Uterine.spasm:3:4851:0:31:0.99:1.00:0.09:0.16), (Vulval.cancer.metastatic:3:4851: 0:31:0.99:1.00:0.09:0.16), (Antidepressant.drug.level.increased:2:4852:0:31:0.99:1.00:0.06:0.11), (Basilar.migraine:2:4852:0:31:0.99:1.00:0.06:0.11), (Coagulation.factor:2:4852:0:31:0.99:1.00:0. 06:0.11), (Hypocoagulable.state:2:4852:0:31:0.99:1.00:0.06:0.11), (Inadequate.lubrication:2:4852: 0:31:0.99:1.00:0.06:0.11), (Infusion.site.cellulitis:2:4852:0:31:0.99:1.00:0.06:0.11), (Ischaemic.li mb.pain:2:4852:0:31:0.99:1.00:0.06:0.11), (Kernicterus:2:4852:0:31:0.99:1.00:0.06:0.11), (Labile.blood.pressure:2:4852:0:31:0.99:1.00:0.06:0.11), (Large.cell.lung.cancer.stage.IV:2:4852:0:31:0. 99:1.00:0.06:0.11), (Marfan's.syndrome:2:4852:0:31:0.99:1.00:0.06:0.11), (Mycosis.fungoides.sta ge.I:2:4852:0:31:0.99:1.00:0.06:0.11), (Myokymia:2:4852:0:31:0.99:1.00:0.06:0.11), (Nuclear.ma gnetic.resonance.imaging.heart:2:4852:0:31:0.99:1.00:0.06:0.11), (Packed.red.blood.cell.transfusi on:2:4852:0:31:0.99:1.00:0.06:0.11), (Polysubstance.dependence:2:4852:0:31:0.99:1.00:0.06:0.1 1), (Renal.cancer.stage.I:2:4852:0:31:0.99:1.00:0.06:0.11), (Retinopathy.proliferative:2:4852:0:31: 0.99:1.00:0.06:0.11), (Rift.Valley.fever:2:4852:0:31:0.99:1.00:0.06:0.11), (Vaginismus:2:4852:0: 31:0.99:1.00:0.06:0.11), (Anosmia:1:4853:0:31:0.99:1.00:0.03:0.06), (Immunology.test:1:4853:0: 31:0.99:1.00:0.03:0.06), (Impaired.quality.of.life:1:4853:0:31:0.99:1.00:0.03:0.06), (Micropenis:1: 4853:0:31:0.99:1.00:0.03:0.06), (Orthopnoea:1:4853:0:31:0.99:1.00:0.03:0.06), (Pulseless.electri cal.activity:1:4853:0:31:0.99:1.00:0.03:0.06), (Respiratory.tract.malformation:1:4853:0:31:0.99:1 0.00:0.03:0.06), (Spider.vein:1:4853:0:31:0.99:1.00:0.03:0.06), (Spinal.cord.abscess:1:4853:0:31:0. 99:1.00:0.03:0.06), (Therapeutic.response.increased:1:4853:0:31:0.99:1.00:0.03:0.06), (Bite:0:485 3:0:32:0.99:#NUM!:0.00:#NUM!), (Blood.creatinine.decreased:0:4853:0:32:0.99:#NUM!:0.00:#NUM!), (Bums.third.degree:0:4853:0:32:0.99:#NUM!:0.00:#NUM!), (Compartment.syndrome:0: 4853:0:32:0.99:#NUM!:0.00:#NUM!), (Dermal.filler.injection:0:4853:0:32:0.99:#NUM!:0.00:#N UM!), (Frustration.tolerance.decreased:0:4853:0:32:0.99:#NUM!:0.00:#NUM!), (Gastrointestinal. arteriovenous.malformation:0:4853:0:32:0.99:#NUM!:0.00:#NUM!), (Gastrointestinal.endoscopi c.therapy:0:4853:0:32:0.99:#NUM!:0.00:#NUM!), (Genital.candidiasis:0:4853:0:32:0.99:#NUM!0.0.00:#NUM!), (Hallucination,.olfactory:0:4853:0:32:0.99:#NUM!:0.00:#NUM!), (Keratitis.bacte rial:0:4853:0:32:0.99:#NUM!:0.00:#NUM!), (Malignant.mast.cell.neoplasm:0:4853:0:32:0.99:#N UM!:0.00:#NUM!), (Metaplasia:0:4853:0:32:0.99:#NUM!:0.00:#NUM!), (Methylmalonic.aciduri a:0:4853:0:32:0.99:#NUM!:0.00:#NUM!), (Multiple.gated.acquisition.scan:0:4853:0:32:0.99:#N UM!:0.00:#NUM!), (Oesophagitis.ulcerative:0:4853:0:32:0.99:#NUM!:0.00:#NUM!), (Renal.arte ry.stent.placement:0:4853:0:32:0.99:#NUM!:0.00:#NUM!), (Stemitis:0:4853:0:32:0.99:#NUM!: 0.00:#NUM!), (Vaginitis.gardnerella:0:4853:0:32:0.99:#NUM!:0.00:#NUM!), (Non-Hodgkin's.lymphoma.stage.III:23:4830:0:32:0.99:1.00:0.42:0.59), (Autoimmune.pancytopenia:18: 4835:0:32:0.99:1.00:0.36:0.53), (B-cell.prolymphocytic.leukaemia:10:4843:0:32:0.99:1.00:0.24:0.38), (JC.virus.test.positive:10:4843: 0:32:0.99:1.00:0.24:0.38), (Testicular.seminoma.(pure):9:4844:0:32:0.99:1.00:0.22:0.36), (Medic al.device.pain:8:4845:0:32:0.99:1.00:0.20:0.33), (Mycosis.fungoides.stage.IV:8:4845:0:32:0.99:1 0.00:0.20:0.33), (Oesophageal.adenocarcinoma.metastatic:8:4845:0:32:0.99:1.00:0.20:0.33), (Testi cular.germ.cell.cancer.metastatic:8:4845:0:32:0.99:1.00:0.20:0.33), (Urticarial.vasculitis:8:4845: 0:32:0.99:1.00:0.20:0.33), (Abdominal.operation:7:4846:0:32:0.99:1.00:0.18:0.30), (Comeal.erosi on:7:4846:0:32:0.99:1.00:0.18:0.30), (Cardiolipin.antibody:6:4847:0:32:0.99:1.00:0.16:0.27), (Ab dominal.hemia.repair:5:4848:0:32:0.99:1.00:0.14:0.24), (Adenosquamous.cell.carcinoma:5:4848: 0:32:0.99:1.00:0.14:0.24), (Autoimmune.uveitis:5:4848:0:32:0.99:1.00:0.14:0.24), (Bone.marrow. infiltration:5:4848:0:32:0.99:1.00:0.14:0.24), (Gait.spastic:5:4848:0:32:0.99:1.00:0.14:0.24), (Oes ophageal.squamous.cell.carcinoma.metastatic:5:4848:0:32:0.99:1.00:0.14:0.24), (pH.body.fluid:5: 4848:0:32:0.99:1.00:0.14:0.24), (Uterine.cyst:5:4848:0:32:0.99:1.00:0.14:0.24), (Dysentery:4:48 49:0:32:0.99:1.00:0.11:0.20), (Friedreich's.ataxia:4:4849:0:32:0.99:1.00:0.11:0.20), (Illusion:4:48 49:0:32:0.99:1.00:0.11:0.20), (Metastases.to.small.intestine:4:4849:0:32:0.99:1.00:0.11:0.20), (Pa roxysmal.arrhythmia:4:4849:0:32:0.99:1.00:0.11:0.20), (Psychomotor.seizures:4:4849:0:32:0.99: 1.00:0.11:0.20), (Thyrotoxic.crisis:4:4849:0:32:0.99:1.00:0.11:0.20), (Typical.aura.without.heada che:4:4849:0:32:0.99:1.00:0.11:0.20), (Ventricular.hypertrophy:4:4849:0:32:0.99:1.00:0.11:0.20), (Apocrine.breast.carcinoma:3:4850:0:32:0.99:1.00:0.09:0.16), (Biliary.sepsis:3:4850:0:32:0.99:1 0.00:0.09:0.16), (Blood.erythropoietin.decreased:3:4850:0:32:0.99:1.00:0.09:0.16), (Eyelid.irritatio n:3:4850:0:32:0.99:1.00:0.09:0.16), (Hepatic.cancer.stage.IV:3:4850:0:32:0.99:1.00:0.09:0.16), (P ost.procedural.discomfort:3:4850:0:32:0.99:1.00:0.09:0.16), (Traumatic.fracture:3:4850:0:32:0.9 9:1.00:0.09:0.16), (Carcinoid.tumour.of.the.pancreas:2:4851:0:32:0.99:1.00:0.06:0.11), (Eye.infec tion.staphylococcal:2:4851:0:32:0.99:1.00:0.06:0.11), (Ovarian.vein.thrombosis:2:4851:0:32:0.99: 1.00:0.06:0.11), (Transgender.operation:2:4851:0:32:0.99:1.00:0.06:0.11), (Bleeding.time:1:4852: 0:32:0.99:1.00:0.03:0.06), (Blood.magnesium.increased:1:4852:0:32:0.99:1.00:0.03:0.06), (Brief. psychotic.disorder,.with.postpartum.onset:1:4852:0:32:0.99:1.00:0.03:0.06), (Cortisol.decreased: 1:4852:0:32:0.99:1.00:0.03:0.06), (Depressive.delusion:1:4852:0:32:0.99:1.00:0.03:0.06), (Haema ngioma.of.liver:1:4852:0:32:0.99:1.00:0.03:0.06), (Hip.deformity:1:4852:0:32:0.99:1.00:0.03:0.0 6), (Hydrotherapy:1:4852:0:32:0.99:1.00:0.03:0.06), (Hyperacusis:1:4852:0:32:0.99:1.00:0.03:0.0 6), (Life.expectancy.shortened:1:4852:0:32:0.99:1.00:0.03:0.06), (Mouth.injury:1:4852:0:32:0.99: 1.00:0.03:0.06), (Nasal.oedema:1:4852:0:32:0.99:1.00:0.03:0.06), (Osteodystrophy:1:4852:0:32:0. 99:1.00:0.03:0.06), (Acute.prerenal.failure:0:4852:0:33:0.99:#NUM!:0.00:#NUM!), (Arterial.byp ass.operation:0:4852:0:33:0.99:#NUM!:0.00:#NUM!), (Hepatic.congestion:0:4852:0:33:0.99:#N UM!:0.00:#NUM!), (Posterior.capsule.opacification:0:4852:0:33:0.99:#NUM!:0.00:#NUM!), (Th alamus.haemorrhage:0:4852:0:33:0.99:#NUM!:0.00:#NUM!), (Ewing's.sarcoma.recurrent:13:483 9:0:33:0.99:1.00:0.28:0.44), (Bone.erosion:9:4843:0:33:0.99:1.00:0.21:0.35), (Hyper.IgD.syndro me:9:4843:0:33:0.99:1.00:0.21:0.35), (Metaplastic.breast.carcinoma:9:4843:0:33:0.99:1.00:0.21: 0.35), (Autologous.bone.marrow.transplantation.therapy:8:4844:0:33:0.99:1.00:0.20:0.33), (Prem ature.ejaculation:8:4844:0:33:0.99:1.00:0.20:0.33), (Retroperitoneal.fibrosis:8:4844:0:33:0.99:1.0 0:0.20:0.33), (Tongue.neoplasm:8:4844:0:33:0.99:1.00:0.20:0.33), (HIV.wasting.syndrome:7:484 5:0:33:0.99:1.00:0.18:0.30), (Viral.diarrhoea:7:4845:0:33:0.99:1.00:0.18:0.30), (Alopecia.scarring: 5:4847:0:33:0.99:1.00:0.13:0.23), (Astrocytoma.malignant:5:4847:0:33:0.99:1.00:0.13:0.23), (Dr ug.level.changed:5:4847:0:33:0.99:1.00:0.13:0.23), (Ejection.fraction:5:4847:0:33:0.99:1.00:0.13: 0.23), (Meningeal.neoplasm:5:4847:0:33:0.99:1.00:0.13:0.23), (Pulmonary.hypoplasia:5:4847:0: 33:0.99:1.00:0.13:0.23), (Strangury:5:4847:0:33:0.99:1.00:0.13:0.23), (Congenital.cardiovascular. anomaly:4:4848:0:33:0.99:1.00:0.11:0.20), (Hereditary.motor.and.sensory.neuropathy:4:4848:0:3 3:0.99:1.00:0.11:0.20), (Prostatic.atrophy:4:4848:0:33:0.99:1.00:0.11:0.20), (Sweat.gland.disorder: 4:4848:0:33:0.99:1.00:0.11:0.20), (Tetanus.immunisation:4:4848:0:33:0.99:1.00:0.11:0.20), (Aph eresis:3:4849:0:33:0.99:1.00:0.08:0.15), (Cryptosporidiosis.infection:3:4849:0:33:0.99:1.00:0.08: 0.15), (Genitourinary.symptom:3:4849:0:33:0.99:1.00:0.08:0.15), (Morton's.neuralgia:3:4849:0:3 3:0.99:1.00:0.08:0.15), (Pelvic.floor.muscle.weakness:3:4849:0:33:0.99:1.00:0.08:0.15), (Postope rative.fever:3:4849:0:33:0.99:1.00:0.08:0.15), (Sexually.inappropriate.behaviour:3:4849:0:33:0.9 9:1.00:0.08:0.15), (Spinal.cord.injury.cervical:3:4849:0:33:0.99:1.00:0.08:0.15), (Brain.stem.strok e:2:4850:0:33:0.99:1.00:0.06:0.11), (Depression.postoperative:2:4850:0:33:0.99:1.00:0.06:0.11), (Drug.effect.increased:2:4850:0:33:0.99:1.00:0.06:0.11), (Lens.extraction:2:4850:0:33:0.99:1.00:0 0.06:0.11), (Neonatal.respiratory.distress.syndrome.prophylaxis:2:4850:0:33:0.99:1.00:0.06:0.11), (Partial.lung.resection:2:4850:0:33:0.99:1.00:0.06:0.11), (Peritoneal.neoplasm:2:4850:0:33:0.99:1. 00:0.06:0.11), (Rotator.cuff.repair:2:4850:0:33:0.99:1.00:0.06:0.11), (Streptococcal.urinary.tract.i nfection:2:4850:0:33:0.99:1.00:0.06:0.1 1), (Subclavian.artery.stenosis:2:4850:0:33:0.99:1.00:0.0 6:0.11), (Synovial.rupture:2:4850:0:33:0.99:1.00:0.06:0.11), (Transferrin.saturation:2:4850:0:33:0 0.99:1.00:0.06:0.11), (Trismus:2:4850:0:33:0.99:1.00:0.06:0.11), (Vulval.disorder:2:4850:0:33:0.9 9:1.00:0.06:0.11), (Allodynia:1:4851:0:33:0.99:1.00:0.03:0.06), (Bradyarrhythmia:1:4851:0:33:0. 99:1.00:0.03:0.06), (Cerebral.circulatory.failure:1:4851:0:33:0.99:1.00:0.03:0.06), (Depilation:1:4 851:0:33:0.99:1.00:0.03:0.06), (Endoscopy.gastrointestinal:1:4851:0:33:0.99:1.00:0.03:0.06), (Fa milial.cold.autoinflammatory.syndrome:1:4851:0:33:0.99:1.00:0.03:0.06), (Gitelman's.syndrome: 1:4851:0:33:0.99:1.00:0.03:0.06), (Nasal.operation:1:4851:0:33:0.99:1.00:0.03:0.06), (Bacterial.v ulvovaginitis:0:4851:0:34:0.99:#NUM!:0.00:#NUM!), (Behavioural.insomnia.of.childhood:0:485 1:0:34:0.99:#NUM!:0.00:#NUM!), (Cardiac.ventricular.disorder:0:4851:0:34:0.99:#NUM!:0.00: #NUM!), (Colonic.pseudo-obstruction:0:4851:0:34:0.99:#NUM!:0.00:#NUM!), (Duodenal.perforation:0:4851:0:34:0.99:#N UM!:0.00:#NUM!), (Early.onset.primary.dystonia:0:4851:0:34:0.99:#NUM!:0.00:#NUM!), (Gast rointestinal.disorder.postoperative:0:4851:0:34:0.99:#NUM!:0.00:#NUM!), (Gastrointestinal.obst ruction:0:4851:0:34:0.99:#NUM!:0.00:#NUM!), (Genital.tract.inflammation:0:4851:0:34:0.99:#NUM!:0.00:#NUM!), (Hunger:0:4851:0:34:0.99:#NUM!:0.00:#NUM!), (Presenile.dementia:0:48 51:0:34:0.99:#NUM!:0.00:#NUM!), (Radiation.associated.pain:0:4851:0:34:0.99:#NUM!:0.00:#NUM!), (Somatoform.disorder:0:4851:0:34:0.99:#NUM!:0.00:#NUM!), (Burkitt's.leukaemia:16:4 835:0:34:0.99:1.00:0.32:0.48), (Bone.sarcoma:14:4837:0:34:0.99:1.00:0.29:0.45), (Frontal.lobe.ep ilepsy:13:4838:0:34:0.99:1.00:0.28:0.43), (HIV.infection.CDC.category.A:13:4838:0:34:0.99:1.0 0:0.28:0.43), (High.grade.B-cell.lymphoma.Burkitt-like.lymphoma:11:4840:0:34:0.99:1.00:0.24:0.39), (High.grade.B-cell.lymphoma.Burkitt-like.lymphoma.stage.IV:11:4840:0:34:0.99:1.00:0.24:0.39), (Lymphatic.disorder:9:4842:0:34:0.9 9:1.00:0.21:0.35), (Peritumoural.oedema:9:4842:0:34:0.99:1.00:0.21:0.35), (Red.blood.cell.sedim entation.rate.increased:9:4842:0:34:0.99:1.00:0.21:0.35), (Alveolar.soft.part.sarcoma:8:4843:0:34: 0.99:1.00:0.19:0.32), (Cholestasis.of.pregnancy:8:4843:0:34:0.99:1.00:0.19:0.32), (Ocular.lymph oma:8:4843:0:34:0.99:1.00:0.19:0.32), (Adrenal.neoplasm:7:4844:0:34:0.99:1.00:0.17:0.29), (Epit helioid.sarcoma:7:4844:0:34:0.99:1.00:0.17:0.29), (Progesterone.receptor.assay.positive:7:4844:0: 34:0.99:1.00:0.17:0.29), (Substance-induced.mood.disorder:7:4844:0:34:0.99:1.00:0.17:0.29), (Drug.specific.antibody.present:6:4845: 0:34:0.99:1.00:0.15:0.26), (Meningitis.haemophilus:6:4845:0:34:0.99:1.00:0.15:0.26), (Myocardi al.bridging:6:4845:0:34:0.99:1.00:0.15:0.26), (Pneumonia.chlamydial:6:4845:0:34:0.99:1.00:0.15: 0.26), (Shunt.infection:6:4845:0:34:0.99:1.00:0.15:0.26), (Drug.administered.to.patient.of.inappr opriate.age:5:4846:0:34:0.99:1.00:0.13:0.23), (HIV.infection.CDC.Group.IV.subgroup.E:5:4846: 0:34:0.99:1.00:0.13:0.23), (Lung.carcinoma.cell.type.unspecified.stage.III:5:4846:0:34:0.99:1.00: 0.13:0.23), (Papillary.serous.endometrial.carcinoma:5:4846:0:34:0.99:1.00:0.13:0.23), (Tongue.di scolouration:5:4846:0:34:0.99:1.00:0.13:0.23), (Abnormal.loss.of.weight:4:4847:0:34:0.99:1.00:0 0.11:0.19), (Fasciitis:4:4847:0:34:0.99:1.00:0.11:0.19), (Hypervitaminosis.D:4:4847:0:34:0.99:1.00: 0.11:0.19), (pH.body.fluid.abnormal:4:4847:0:34:0.99:1.00:0.11:0.19), (Porphyria.non-acute:4:4847:0:34:0.99:1.00:0.11:0.19), (Sticky.platelet.syndrome:4:4847:0:34:0.99:1.00:0.11:0.1 9), (Benign.neoplasm.of.thyroid.gland:3:4848:0:34:0.99:1.00:0.08:0.15), (Bile.duct.stenosis:3:484 8:0:34:0.99:1.00:0.08:0.15), (Brain.stem.haemorrhage:3:4848:0:34:0.99:1.00:0.08:0.15), (Breast.o peration:3:4848:0:34:0.99:1.00:0.08:0.15), (Food.craving:3:4848:0:34:0.99:1.00:0.08:0.15), (Foot. amputation:3:4848:0:34:0.99:1.00:0.08:0.15), (Hepatobiliary.disease:3:4848:0:34:0.99:1.00:0.08: 0.15), (Nelson's.syndrome:3:4848:0:34:0.99:1.00:0.08:0.15), (Parkinsonian.gait:3:4848:0:34:0.99: 1.00:0.08:0.15), (Pulmonary.valve.stenosis:3:4848:0:34:0.99:1.00:0.08:0.15), (Salivary.gland.disorder: 3:4848:0:34:0.99:1.00:0.08:0.15), (Surgical.failure:3:4848:0:34:0.99:1.00:0.08:0.15), (Tumour. necrosis:3:4848:0:34:0.99:1.00:0.08:0.15), (Candida.test.positive:2:4849:0:34:0.99:1.00:0.06:0. 11), (Diphtheria:2:4849:0:34:0.99:1.00:0.06:0.11), (Heart.injury:2:4849:0:34:0.99:1.00:0.06:0.11), (Medical.device.site.pain:2:4849:0:34:0.99:1.00:0.06:0.11), (Peritoneal.candidiasis:2:4849:0:34: 0.99:1.00:0.06:0.11), (Stoma.site.reaction:2:4849:0:34:0.99:1.00:0.06:0.11), (Trabeculectomy:2:4 849:0:34:0.99:1.00:0.06:0.11), (Adenoiditis:1:4850:0:34:0.99:1.00:0.03:0.06), (Antipsychotic.dru g.level:1:4850:0:34:0.99:1.00:0.03:0.06), (Camitine:1:4850:0:34:0.99:1.00:0.03:0.06), (Cestode.in fection:1:4850:0:34:0.99:1.00:0.03:0.06), (Ketoacidosis:1:4850:0:34:0.99:1.00:0.03:0.06), (Learni ng.disorder:1:4850:0:34:0.99:1.00:0.03:0.06), (Lichenification:1:4850:0:34:0.99:1.00:0.03:0.06), (Purpura.fulminans:1:4850:0:34:0.99:1.00:0.03:0.06), (Salivary.gland.cancer.recurrent:1:4850:0:3 4:0.99:1.00:0.03:0.06), (Steatorrhoea:1:4850:0:34:0.99:1.00:0.03:0.06), (Benign.neoplasm.of.thy mus:0:4850:0:35:0.99:#NUM!:0.00:#NUM!), (Congenital.hepatic.fibrosis:0:4850:0:35:0.99:#NU M!:0.00:#NUM!), (Dislocation.of.vertebra:0:4850:0:35:0.99:#NUM!:0.00:#NUM!), (Enterobacter.test.positive:0:4850:0:35:0.99:#NUM!:0.00:#NUM!), (Gastric.banding:0:4850:0:35:0.99:#NUM!: 0.00:#NUM!), (Medical.induction.of.coma:0:4850:0:35:0.99:#NUM!:0.00:#NUM!), (Meibomian itis:0:4850:0:35:0.99:#NUM!:0.00:#NUM!), (Molluscum.contagiosum:0:4850:0:35:0.99:#NUM!: 0.00:#NUM!), (Paracentesis:0:4850:0:35:0.99:#NUM!:0.00:#NUM!), (Patient-device.incompatibility:0:4850:0:35:0.99:#NUM!:0.00:#NUM!), (Tooth.repair:0:4850:0:35:0.99:#NUM!:0.00:#NUM!), (Exposure.via.father:18:4832:0:35:0.99:1.00:0.34:0.51), (T-cell.lymphoma.recurrent:14:4836:0:35:0.99:1.00:0.29:0.44), (Immunoglobulin.G4.related.disease: 11:4839:0:35:0.99:1.00:0.24:0.39), (Genitourinary.tract.neoplasm:10:4840:0:35:0.99:1.00:0.22:0 0.36), (Extrapulmonary.tuberculosis:9:4841:0:35:0.99:1.00:0.20:0.34), (Inflammatory.myofibroblas tic.tumour:7:4843:0:35:0.99:1.00:0.17:0.29), (Papule:7:4843:0:35:0.99:1.00:0.17:0.29), (Therapeu tic.response.decreased:7:4843:0:35:0.99:1.00:0.17:0.29), (Central.nervous.system.inflammation:6:4844:0:35:0.99:1.00:0.15:0.26), (Ecthyma:6:4844:0:35:0.99:1.00:0.15:0.26), (Congenital.nephrot ic.syndrome:5:4845:0:35:0.99:1.00:0.13:0.22), (Malignant.neoplasm.of.choroid:5:4845:0:35:0.99: 1.00:0.13:0.22), (Peripartum.cardiomyopathy:5:4845:0:35:0.99:1.00:0.13:0.22), (Peripheral.arter y.stent.insertion:5:4845:0:35:0.99:1.00:0.13:0.22), (Small.intestine.ulcer:5:4845:0:35:0.99:1.00:0. 13:0.22), (Bone.cyst:4:4846:0:35:0.99:1.00:0.10:0.19), (Glaucoma.surgery:4:4846:0:35:0.99:1.00: 0.10:0.19), (Immobilisation.prolonged:4:4846:0:35:0.99:1.00:0.10:0.19), (Mesenteric.artery.throm bosis:4:4846:0:35:0.99:1.00:0.10:0.19), (Prerenal.failure:4:4846:0:35:0.99:1.00:0.10:0.19), (Priapi sm:4:4846:0:35:0.99:1.00:0.10:0.19), (Silicosis:4:4846:0:35:0.99:1.00:0.10:0.19), (Blood.folate.a bnormal:3:4847:0:35:0.99:1.00:0.08:0.15), (Eschar:3:4847:0:35:0.99:1.00:0.08:0.15), (Fibrin.degr adation.products.increased:3:4847:0:35:0.99:1.00:0.08:0.15), (Limb.immobilisation:3:4847:0:35: 0.99:1.00:0.08:0.15), (Paraphilia:3:4847:0:35:0.99:1.00:0.08:0.15), (Peritoneal.sarcoma:3:4847:0: 35:0.99:1.00:0.08:0.15), (Rectal.prolapse:3:4847:0:35:0.99:1.00:0.08:0.15), (Blood.thrombin:2:48 48:0:35:0.99:1.00:0.05:0.10), (Erosive.duodenitis:2:4848:0:35:0.99:1.00:0.05:0.10), (Fistula.repai r:2:4848:0:35:0.99:1.00:0.05:0.10), (Keratomileusis:2:4848:0:35:0.99:1.00:0.05:0.10), (Lacunar.st roke:2:4848:0:35:0.99:1.00:0.05:0.10), (Lower.respiratory.tract.infection.viral:2:4848:0:35:0.99:1 0.00:0.05:0.10), (Noonan.syndrome:2:4848:0:35:0.99:1.00:0.05:0.10), (Phlebotomy:2:4848:0:35:0. 99:1.00:0.05:0.10), (Sclerotherapy:2:4848:0:35:0.99:1.00:0.05:0.10), (Sinus.polyp:2:4848:0:35:0. 99:1.00:0.05:0.10), (Abdominal.fat.apron:1:4849:0:35:0.99:1.00:0.03:0.05), (Aortic.disorder:1:48 49:0:35:0.99:1.00:0.03:0.05), (Aortic.valve.repair:1:4849:0:35:0.99:1.00:0.03:0.05), (Astringent.t herapy:1:4849:0:35:0.99:1.00:0.03:0.05), (Bladder.ablation:1:4849:0:35:0.99:1.00:0.03:0.05), (Hy perchloraemia:1:4849:0:35:0.99:1.00:0.03:0.05), (Ingrown.hair:1:4849:0:35:0.99:1.00:0.03:0.05), (Microvascular.coronary.artery.disease:1:4849:0:35:0.99:1.00:0.03:0.05), (Traumatic.arthritis:1:4 849:0:35:0.99:1.00:0.03:0.05), (Vasogenic.cerebral.oedema:1:4849:0:35:0.99:1.00:0.03:0.05), (He lminthic.infection:0:4849:0:36:0.99:#NUM!:0.00:#NUM!), (Prophylaxis.against.motion.sickness: 0:4849:0:36:0.99:#NUM!:0.00:#NUM!), (Prostatic.obstruction:0:4849:0:36:0.99:#NUM!:0.00:#NUM!), (Schizoid.personality.disorder:0:4849:0:36:0.99:#NUM!:0.00:#NUM!), (Tongue.fungal.i nfection:0:4849:0:36:0.99:#NUM!:0.00:#NUM!), (Lymphoma.AIDS.related:32:4817:1:35:0.99:0 0.97:0.48:0.64), (Refractory.cancer:17:4832:0:36:0.99:1.00:0.32:0.49), (Blastic.plasmacytoid.dendr itic.cell.neoplasia:13:4836:0:36:0.99:1.00:0.27:0.42), (Nodal.marginal.zone.B-cell.lymphoma:13:4836:0:36:0.99:1.00:0.27:0.42), (Bicytopenia:9:4840:0:36:0.99:1.00:0.20:0.33), (Cardiac.septal.defect:9:4840:0:36:0.99:1.00:0.20:0.33), (Inflammatory.marker.increased:9:484 0:0:36:0.99:1.00:0.20:0.33), (Serum.sickness:9:4840:0:36:0.99:1.00:0.20:0.33), (Vasculitis.necroti sing:9:4840:0:36:0.99:1.00:0.20:0.33), (Gastroenteropancreatic.neuroendocrine.tumour.disease:8: 4841:0:36:0.99:1.00:0.18:0.31), (Glomerulonephropathy:8:4841:0:36:0.99:1.00:0.18:0.31), (C-reactive.protein:7:4842:0:36:0.99:1.00:0.16:0.28), (Leukoencephalopathy:7:4842:0:36:0.99:1.00: 0.16:0.28), (Pulmonary.artery.stenosis:7:4842:0:36:0.99:1.00:0.16:0.28), (Ileectomy:6:4843:0:36: 0.99:1.00:0.14:0.25), (Phyllodes.tumour:6:4843:0:36:0.99:1.00:0.14:0.25), (Abstains.from.alcohol: 5:4844:0:36:0.99:1.00:0.12:0.22), (Complications.of.transplanted.heart:5:4844:0:36:0.99:1.00:0. 12:0.22), (Gastroenteritis.Escherichia.coli:5:4844:0:36:0.99:1.00:0.12:0.22), (Vertebral.foraminal. stenosis:5:4844:0:36:0.99:1.00:0.12:0.22), (Eye.infection.viral:4:4845:0:36:0.99:1.00:0.10:0.18), (Lip.disorder:4:4845:0:36:0.99:1.00:0.10:0.18), (Cryofibrinogenaemia:3:4846:0:36:0.99:1.00:0.08: 0.14), (Duodenal.ulcer.perforation:3:4846:0:36:0.99:1.00:0.08:0.14), (Gastrointestinal.ulcer.haem orrhage:3:4846:0:36:0.99:1.00:0.08:0.14), (Peripheral.nervous.system.function.test.abnormal:3:4 846:0:36:0.99:1.00:0.08:0.14), (Therapeutic.response.unexpected:3:4846:0:36:0.99:1.00:0.08:0.1 4), (Urethritis.noninfective:3:4846:0:36:0.99:1.00:0.08:0.14), (Venous.injury:3:4846:0:36:0.99:1. 00:0.08:0.14), (Anal.cancer.stage.IV:2:4847:0:36:0.99:1.00:0.05:0.10), (Band.sensation:2:4847:0: 36:0.99:1.00:0.05:0.10), (Body.mass.index.increased:2:4847:0:36:0.99:1.00:0.05:0.10), (Calculus. bladder:2:4847:0:36:0.99:1.00:0.05:0.10), (Dacryocystitis:2:4847:0:36:0.99:1.00:0.05:0.10), (Met astatic.salivary.gland.cancer:2:4847:0:36:0.99:1.00:0.05:0.10), (Neutrophil.function.disorder:2:48 47:0:36:0.99:1.00:0.05:0.10), (Non-small.cell.lung.cancer.stage.II:2:4847:0:36:0.99:1.00:0.05:0.10), (Oesophageal.perforation:2:484 7:0:36:0.99:1.00:0.05:0.10), (Cardiorenal.syndrome:1:4848:0:36:0.99:1.00:0.03:0.05), (Gingival.r ecession:1:4848:0:36:0.99:1.00:0.03:0.05), (Labile.hypertension:1:4848:0:36:0.99:1.00:0.03:0.05), (Naevus.flammeus:1:4848:0:36:0.99:1.00:0.03:0.05), (Premature.separation.of.placenta:1:4848: 0:36:0.99:1.00:0.03:0.05), (Pseudocyst:1:4848:0:36:0.99:1.00:0.03:0.05), (Toxic.shock.syndrome. streptococcal:1:4848:0:36:0.99:1.00:0.03:0.05), (Transfusion.related.complication:1:4848:0:36:0. 99:1.00:0.03:0.05), (Urticaria.cholinergic:1:4848:0:36:0.99:1.00:0.03:0.05), (Bladder.neck.suspen sion:0:4848:0:37:0.99:#NUM!:0.00:#NUM!), (Collateral.circulation:0:4848:0:37:0.99:#NUM!:0. 00:#NUM!), (Face.lift:0:4848:0:37:0.99:#NUM!:0.00:#NUM!), (Keratopathy:0:4848:0:37:0.99:#NUM!:0.00:#NUM!), (Lumbar.hernia:0:4848:0:37:0.99:#NUM!:0.00:#NUM!), (Sebaceous.gland. disorder:0:4848:0:37:0.99:#NUM!:0.00:#NUM!), (Asymptomatic.HIV.infection:18:4830:0:37:0. 99:1.00:0.33:0.49), (Hodgkin's.disease.refractory:16:4832:0:37:0.99:1.00:0.30:0.46), (Rectal.canc er.stage.Ill:14:4834:0:37:0.99:1.00:0.27:0.43), (Rheumatoid.factor.positive:11:4837:0:37:0.99:1. 00:0.23:0.37), (Aspergillus.test:8:4840:0:37:0.99:1.00:0.18:0.30), (Intestinal.fistula:8:4840:0:37:0. 99:1.00:0.18:0.30), (Infection.susceptibility.increased:7:4841:0:37:0.99:1.00:0.16:0.27), (Cervix.c arcinoma.stage.IV:6:4842:0:37:0.99:1.00:0.14:0.24), (Endometrial.stromal.sarcoma:6:4842:0:37: 0.99:1.00:0.14:0.24), (Hyperparathyroidism.tertiary:6:4842:0:37:0.99:1.00:0.14:0.24), (Hyperpitui tarism:6:4842:0:37:0.99:1.00:0.14:0.24), (Invasive.papillary.breast.carcinoma:6:4842:0:37:0.99:1 0.00:0.14:0.24), (Mental.retardation:6:4842:0:37:0.99:1.00:0.14:0.24), (Amoebic.dysentery:5:4843: 0:37:0.99:1.00:0.12:0.21), (Cervix.carcinoma.recurrent:5:4843:0:37:0.99:1.00:0.12:0.21), (Postpar tum.hypopituitarism:5:4843:0:37:0.99:1.00:0.12:0.21), (Debridement:4:4844:0:37:0.99:1.00:0.10: 0.18), (Hypoplastic.left.heart.syndrome:4:4844:0:37:0.99:1.00:0.10:0.18), (Medical.device.implan tation:4:4844:0:37:0.99:1.00:0.10:0.18), (Noninfective.encephalitis:4:4844:0:37:0.99:1.00:0.10:0. 18), (Physiotherapy:4:4844:0:37:0.99:1.00:0.10:0.18), (Product.solubility.abnormal:4:4844:0:37:0 0.99:1.00:0.10:0.18), (Chemotherapy.urothelial.toxicity.attenuation:3:4845:0:37:0.99:1.00:0.08:0.1 4), (Decreased.activity:3:4845:0:37:0.99:1.00:0.08:0.14), (Hereditary.disorder:3:4845:0:37:0.99:1. 00:0.08:0.14), (Sucrase-isomaltase.deficiency:3:4845:0:37:0.99:1.00:0.08:0.14), (Temperature.regulation.disorder:3:4845:0:37:0.99:1.00:0.08:0.14), (Urethral.spasm:3:4845:0:37:0.99:1.00:0.08:0.14), (Vitamin.D.increas ed:3:4845:0:37:0.99:1.00:0.08:0.14), (Vitamin.E:3:4845:0:37:0.99:1.00:0.08:0.14), (Appendiceal. abscess:2:4846:0:37:0.99:1.00:0.05:0.10), (Artificial.crown.procedure:2:4846:0:37:0.99:1.00:0.0 5:0.10), (B-cell.lymphoma.stage.I:2:4846:0:37:0.99:1.00:0.05:0.10), (Beta.2.microglobulin.increased:2:4846: 0:37:0.99:1.00:0.05:0.10), (Breast.inflammation:2:4846:0:37:0.99:1.00:0.05:0.10), (Intentional.pr oduct.use.issue:2:4846:0:37:0.99:1.00:0.05:0.10), (MELAS.syndrome:2:4846:0:37:0.99:1.00:0.05:0.10), (Nodal.rhythm:2:4846:0:37:0.99:1.00:0.05:0.10), (Starvation:2:4846:0:37:0.99:1.00:0.05:0 0.10), (Tyrosinaemia:2:4846:0:37:0.99:1.00:0.05:0.10), (Bacterial.abscess.central.nervous.system:1: 4847:0:37:0.99:1.00:0.03:0.05), (Blood.sodium.abnormal:1:4847:0:37:0.99:1.00:0.03:0.05), (Cre atinine.renal.clearance.decreased:1:4847:0:37:0.99:1.00:0.03:0.05), (Drug.tolerance.increased:1:4 847:0:37:0.99:1.00:0.03:0.05), (Gastric.perforation:1:4847:0:37:0.99:1.00:0.03:0.05), (Infective.gl ossitis:1:4847:0:37:0.99:1.00:0.03:0.05), (Jejunostomy:1:4847:0:37:0.99:1.00:0.03:0.05), (Neutro phil.count.increased:1:4847:0:37:0.99:1.00:0.03:0.05), (Pancreatic.abscess:1:4847:0:37:0.99:1.00: 0.03:0.05), (Parathyroid.tumour:1:4847:0:37:0.99:1.00:0.03:0.05), (Pneumoperitoneum:1:4847:0: 37:0.99:1.00:0.03:0.05), (Polyneuropathy.in.malignant.disease:1:4847:0:37:0.99:1.00:0.03:0.05), (Sphincter.of.Oddi.dysfunction:1:4847:0:37:0.99:1.00:0.03:0.05), (Thoracostomy:1:4847:0:37:0.9 9:1.00:0.03:0.05), (Vipoma:1:4847:0:37:0.99:1.00:0.03:0.05), (Blood.thyroid.stimulating.hormon e.normal:0:4847:0:38:0.99:#NUM!:0.00:#NUM!), (Cardiospasm:0:4847:0:38:0.99:#NUM!:0.00: #NUM!), (Chest.scan:0:4847:0:38:0.99:#NUM!:0.00:#NUM!), (Vitamin.B6.decreased:0:4847:0:3 8:0.99:#NUM!:0.00:#NUM!), (Plasmablastic.lymphoma:27:4820:0:38:0.99:1.00:0.42:0.59), (Emb ryonal.rhabdomyosarcoma:15:4832:0:38:0.99:1.00:0.28:0.44), (Glomerulonephritis.proliferative: 15:4832:0:38:0.99:1.00:0.28:0.44), (Cytomegalovirus.enteritis:13:4834:0:38:0.99:1.00:0.25:0.41), (Kidney.angiomyolipoma:13:4834:1:37:0.99:0.93:0.26:0.41), (Retinoblastoma:13:4834:0:38:0.9 9:1.00:0.25:0.41), (Seminoma:11:4836:0:38:0.99:1.00:0.22:0.37), (Bacterial.pyelonephritis:8:483 9:0:38:0.99:1.00:0.17:0.30), (Large.granular.lymphocytosis:7:4840:0:38:0.99:1.00:0.16:0.27), (Ep idermolysis.bullosa:6:4841:0:38:0.99:1.00:0.14:0.24), (Granulocyte.count.increased:6:4841:0:38: 0.99:1.00:0.14:0.24), (Hepatocellular.injury:5:4842:0:38:0.99:1.00:0.12:0.21), (Hyperferritinaemi a:5:4842:0:38:0.99:1.00:0.12:0.21), (Renal.infarct:5:4842:0:38:0.99:1.00:0.12:0.21), (Benign.bon e.neoplasm:4:4843:0:38:0.99:1.00:0.10:0.17), (Coagulation.factor.V.level.abnormal:4:4843:0:38: 0.99:1.00:0.10:0.17), (Deficiency.anaemia:4:4843:0:38:0.99:1.00:0.10:0.17), (Obesity.surgery:4:4 843:0:38:0.99:1.00:0.10:0.17), (Red.blood.cell.count.increased:4:4843:0:38:0.99:1.00:0.10:0.17), (Thyroid.cyst:4:4843:0:38:0.99:1.00:0.10:0.17), (Urine.uric.acid:4:4843:0:38:0.99:1.00:0.10:0.17), (Lip.neoplasm.malignant.stage.unspecified:3:4844:0:38:0.99:1.00:0.07:0.14), (Malignant.nipple.neoplasm.female:3:4844:0:38:0.99:1.00:0.07:0.14), (Blood.homocysteine.abnormal:2:4845:0:38: 0.99:1.00:0.05:0.10), (Brain.hypoxia:2:4845:0:38:0.99:1.00:0.05:0.10), (Congenital.high.airway.o bstruction.syndrome:2:4845:0:38:0.99:1.00:0.05:0.10), (Endometrial.disorder:2:4845:0:38:0.99:1. 00:0.05:0.10), (Gastrointestinal.melanoma:2:4845:0:38:0.99:1.00:0.05:0.10), (Huerthle.cell.carcin oma:2:4845:0:38:0.99:1.00:0.05:0.10), (Infantile.haemangioma:2:4845:0:38:0.99:1.00:0.05:0.10), (Keratoconus:2:4845:0:38:0.99:1.00:0.05:0.10), (Learning.disability:2:4845:0:38:0.99:1.00:0.05: 0.10), (Nicotinamide.decreased:2:4845:0:38:0.99:1.00:0.05:0.10), (Transcatheter.aortic.valve.impl antation:2:4845:0:38:0.99:1.00:0.05:0.10), (Diplegia:1:4846:0:38:0.99:1.00:0.03:0.05), (Hypopara thyroidism.secondary:1:4846:0:38:0.99:1.00:0.03:0.05), (Mycosis.fungoides.stage.III:1:4846:0:3 8:0.99:1.00:0.03:0.05), (Myomectomy:1:4846:0:38:0.99:1.00:0.03:0.05), (Oculogyric.crisis:1:484 6:0:38:0.99:1.00:0.03:0.05), (Otitis.media.chronic:1:4846:0:38:0.99:1.00:0.03:0.05), (Paraganglio n.neoplasm.malignant:1:4846:0:38:0.99:1.00:0.03:0.05), (Photopsia:1:4846:0:38:0.99:1.00:0.03:0 0.05), (Polyneuropathy.alcoholic:1:4846:0:38:0.99:1.00:0.03:0.05), (Potentiating.drug.interaction:1: 4846:0:38:0.99:1.00:0.03:0.05), (Prostate.tenderness:1:4846:0:38:0.99:1.00:0.03:0.05), (Radiculit is.brachial:1:4846:0:38:0.99:1.00:0.03:0.05), (Tooth.loss:1:4846:0:38:0.99:1.00:0.03:0.05), (Trich otillomania:1:4846:0:38:0.99:1.00:0.03:0.05), (Angiogram.cerebral:0:4846:0:39:0.99:#NUM!:0.0 0:#NUM!), (Atrioventricular.block.first.degree:0:4846:0:39:0.99:#NUM!:0.00:#NUM!), (Brachial.plexus.injury:0:4846:0:39:0.99:#NUM!:0.00:#NUM!), (Fragile.X.syndrome:0:4846:0:39:0.99:#NUM!:0.00:#NUM!), (Hair.colour.changes:0:4846:0:39:0.99:#NUM!:0.00:#NUM!), (Laser.thera py:0:4846:0:39:0.99:#NUM!:0.00:#NUM!), (Radicular.syndrome:0:4846:0:39:0.99:#NUM!:0.00: #NUM!), (Tongue.disorder:0:4846:0:39:0.99:#NUM!:0.00:#NUM!), (Mediastinal.mass:20:4826:0: 39:0.99:1.00:0.34:0.51), (Retinal.vasculitis:13:4833:0:39:0.99:1.00:0.25:0.40), (Birdshot.choriore tinopathy:12:4834:0:39:0.99:1.00:0.24:0.38), (Induced.labour:11:4835:0:39:0.99:1.00:0.22:0.36), (Chronic.myeloid.leukaemia.transformation:10:4836:0:39:0.99:1.00:0.20:0.34), (Fallot's.tetralogy: 8:4838:0:39:0.99:1.00:0.17:0.29), (Skin.graft:8:4838:0:39:0.99:1.00:0.17:0.29), (Complications.o f.transplanted.liver:7:4839:0:39:0.99:1.00:0.15:0.26), (Superior.vena.cava.syndrome:7:4839:0:39: 0.99:1.00:0.15:0.26), (Haematocrit:6:4840:0:39:0.99:1.00:0.13:0.24), (Induction.of.cervix.ripenin g:6:4840:0:39:0.99:1.00:0.13:0.24), (Cataract.nuclear:5:4841:0:39:0.99:1.00:0.11:0.20), (Renal.a myloidosis:5:4841:0:39:0.99:1.00:0.11:0.20), (Leukaemia.monocytic:4:4842:0:39:0.99:1.00:0.09: 0.17), (Metastatic.ocular.melanoma:4:4842:0:39:0.99:1.00:0.09:0.17), (Oncologic.complication:4: 4842:0:39:0.99:1.00:0.09:0.17), (Orthostatic.tremor:4:4842:0:39:0.99:1.00:0.09:0.17), (Polyomavi rus.test.positive:4:4842:0:39:0.99:1.00:0.09:0.17), (Renal.cancer.stage.III:4:4842:0:39:0.99:1.00: 0.09:0.17), (B-cell.small.lymphocytic.lymphoma.recurrent:3:4843:0:39:0.99:1.00:0.07:0.13), (Optic.nerve.disor der:3:4843:0:39:0.99:1.00:0.07:0.13), (Pulmonary.artery.atresia:3:4843:0:39:0.99:1.00:0.07:0.13), (Shoulder.arthroplasty:3:4843:0:39:0.99:1.00:0.07:0.13), (Tongue.ulceration:3:4843:0:39:0.99:1. 00:0.07:0.13), (Biopsy.lung:2:4844:0:39:0.99:1.00:0.05:0.09), (Culture.urine.positive:2:4844:0:39: 0.99:1.00:0.05:0.09), (Dietary.supplement.prescribing.error:2:4844:0:39:0.99:1.00:0.05:0.09), (H yponatraemic.syndrome:2:4844:0:39:0.99:1.00:0.05:0.09), (Hypovolaemic.shock:2:4844:0:39:0.9 9:1.00:0.05:0.09), (Immunoglobulins.abnormal:2:4844:0:39:0.99:1.00:0.05:0.09), (Intensive.care. unit.acquired.weakness:2:4844:0:39:0.99:1.00:0.05:0.09), (Mediastinoscopy:2:4844:0:39:0.99:1.0 0:0.05:0.09), (Motor.neurone.disease:2:4844:0:39:0.99:1.00:0.05:0.09), (Terminal.insomnia:2:484 4:0:39:0.99:1.00:0.05:0.09), (Thyroid.hormones.increased:2:4844:0:39:0.99:1.00:0.05:0.09), (Ves tibular.neuronitis:2:4844:0:39:0.99:1.00:0.05:0.09), (Enterococcal.sepsis:1:4845:0:39:0.99:1.00:0 0.03:0.05), (Genital.discharge:1:4845:0:39:0.99:1.00:0.03:0.05), (Long.QT.syndrome:1:4845:0:39: 0.99:1.00:0.03:0.05), (Nail.dystrophy:1:4845:0:39:0.99:1.00:0.03:0.05), (Severe.acute.respiratory. syndrome:1:4845:0:39:0.99:1.00:0.03:0.05), (Exercise.lack.of:0:4845:0:40:0.99:#NUM!:0.00:#N UM!), (Madarosis:0:4845:0:40:0.99:#NUM!:0.00:#NUM!), (Phlebectomy:0:4845:0:40:0.99:#NU M!:0.00:#NUM!), (Skin.neoplasm.excision:0:4845:0:40:0.99:#NUM!:0.00:#NUM!), (Stent.malfu nction:0:4845:0:40:0.99:#NUM!:0.00:#NUM!), (Central.nervous.system.leukaemia:22:4823:0:40:0.99:1.00:0.35:0.52), (Epstein-Barr.viraemia:16:4829:0:40:0.99:1.00:0.29:0.44), (Primary.effusion.lymphoma:15:4830:0:40:0.9 9:1.00:0.27:0.43), (Cardiac.sarcoidosis:12:4833:0:40:0.99:1.00:0.23:0.38), (Paraneoplastic.pemph igus:12:4833:0:40:0.99:1.00:0.23:0.38), (Gallbladder.adenocarcinoma:11:4834:0:40:0.99:1.00:0. 22:0.35), (Ocular.myasthenia:10:4835:0:40:0.99:1.00:0.20:0.33), (Pneumocystis.test.positive:10:4 835:0:40:0.99:1.00:0.20:0.33), (Abortion:9:4836:0:40:0.99:1.00:0.18:0.31), (Hepatitis.infectious:9: 4836:0:40:0.99:1.00:0.18:0.31), (Choroid.melanoma:8:4837:0:40:0.99:1.00:0.17:0.29), (Steroid. withdrawal.syndrome:8:4837:0:40:0.99:1.00:0.17:0.29), (Retinopathy.of.prematurity:7:4838:0:40: 0.99:1.00:0.15:0.26), (Sporotrichosis:7:4838:1:39:0.99:0.88:0.15:0.26), (Haemorrhage.subcutane ous:6:4839:0:40:0.99:1.00:0.13:0.23), (Paraneoplastic.neurological.syndrome:6:4839:0:40:0.99:1. 00:0.13:0.23), (Foetal.growth.restriction:5:4840:0:40:0.99:1.00:0.11:0.20), (Adrenal.suppression:4: 4841:0:40:0.99:1.00:0.09:0.17), (Calculus.ureteric:4:4841:0:40:0.99:1.00:0.09:0.17), (Carbohydr ate.intolerance:4:4841:0:40:0.99:1.00:0.09:0.17), (Enzyme.inhibition:4:4841:0:40:0.99:1.00:0.09: 0.17), (Fibrinolysis:4:4841:0:40:0.99:1.00:0.09:0.17), (Intracranial.hypotension:4:4841:0:40:0.99: 1.00:0.09:0.17), (Lymphangioma:4:4841:0:40:0.99:1.00:0.09:0.17), (Megacolon:4:4841:0:40:0.99: 1.00:0.09:0.17), (Perirectal.abscess:4:4841:0:40:0.99:1.00:0.09:0.17), (Activities.of.daily.living.i mpaired:3:4842:0:40:0.99:1.00:0.07:0.13), (Device.malfunction:3:4842:0:40:0.99:1.00:0.07:0.13), (Eyelid.rash:3:4842:0:40:0.99:1.00:0.07:0.13), (Fibrin.D.dimer.increased:3:4842:0:40:0.99:1.00: 0.07:0.13), (Meibomian.gland.dysfunction:3:4842:0:40:0.99:1.00:0.07:0.13), (Meningitis.aseptic: 3:4842:0:40:0.99:1.00:0.07:0.13), (Nitrate.compound.therapy:3:4842:0:40:0.99:1.00:0.07:0.13), (Osteotomy:3:4842:0:40:0.99:1.00:0.07:0.13), (Retinal.vascular.occlusion:3:4842:0:40:0.99:1.00: 0.07:0.13), (Retinitis.pigmentosa:3:4842:0:40:0.99:1.00:0.07:0.13), (Subdiaphragmatic.abscess:3: 4842:0:40:0.99:1.00:0.07:0.13), (Tetany:3:4842:0:40:0.99:1.00:0.07:0.13), (Antisocial.personality.disorder:2:4843:0:40:0.99:1.00:0.05:0.09), (Hypoglycaemic.coma:2:4843:0:40:0.99:1.00:0.05:0. 09), (Pulpitis.dental:2:4843:0:40:0.99:1.00:0.05:0.09), (Shunt.stenosis:2:4843:0:40:0.99:1.00:0.05: 0.09), (Sports.injury:2:4843:0:40:0.99:1.00:0.05:0.09), (Vaginal.laceration:2:4843:0:40:0.99:1.00: 0.05:0.09), (Acute.stress.disorder:1:4844:0:40:0.99:1.00:0.02:0.05), (Asphyxia:1:4844:0:40:0.99: 1.00:0.02:0.05), (Cardiac.resynchronisation.therapy:1:4844:0:40:0.99:1.00:0.02:0.05), (Muscle.ha emorrhage:1:4844:0:40:0.99:1.00:0.02:0.05), (Open.fracture:1:4844:0:40:0.99:1.00:0.02:0.05), (P ancreatic.enzymes.abnormal:1:4844:0:40:0.99:1.00:0.02:0.05), (Radioisotope.scan:1:4844:0:40:0. 99:1.00:0.02:0.05), (Renovascular.hypertension:1:4844:0:40:0.99:1.00:0.02:0.05), (Vascular.parki nsonism:1:4844:0:40:0.99:1.00:0.02:0.05), (Vena.cava.filter.insertion:1:4844:0:40:0.99:1.00:0.02: 0.05), (Vitamin.C.abnormal:1:4844:0:40:0.99:1.00:0.02:0.05), (Autonomic.seizure:0:4844:0:41:0 0.99:#NUM!:0.00:#NUM!), (Delayed.sleep.phase:0:4844:0:41:0.99:#NUM!:0.00:#NUM!), (Disrup tive.mood.dysregulation.disorder:0:4844:0:41:0.99:#NUM!:0.00:#NUM!), (Superficial.injury.of eye:0:4844:0:41:0.99:#NUM!:0.00:#NUM!), (Tonsillectomy:0:4844:0:41:0.99:#NUM!:0.00:#NU M!), (Tooth.discolouration:0:4844:0:41:0.99:#NUM!:0.00:#NUM!), (Gestational.trophoblastic.tu mour:18:4826:0:41:0.99:1.00:0.31:0.47), (Metastases.to.abdominal.cavity:18:4826:0:41:0.99:1.0 0:0.31:0.47), (Neurocysticercosis:11:4833:0:41:0.99:1.00:0.21:0.35), (Amniotic.cavity.infection:9: 4835:0:41:0.99:1.00:0.18:0.31), (Antibody.test.abnormal:9:4835:0:41:0.99:1.00:0.18:0.31), (Met astases.to.diaphragm:8:4836:0:41:0.99:1.00:0.16:0.28), (Transitional.cell.cancer.of.renal.pelvis.a nd.ureter.metastatic:7:4837:0:41:0.99:1.00:0.15:0.25), (Adrenal.mass:6:4838:0:41:0.99:1.00:0.13: 0.23), (Laryngeal.pain:6:4838:0:41:0.99:1.00:0.13:0.23), (Ovarian.epithelial.cancer.recurrent:6:48 38:0:41:0.99:1.00:0.13:0.23), (Eye.movement.disorder:5:4839:0:41:0.99:1.00:0.11:0.20), (Blast.cell. count.increased:4:4840:0:41:0.99:1.00:0.09:0.16), (Bone.deformity:4:4840:0:41:0.99:1.00:0.09: 0.16), (Intestinal.perforation:4:4840:0:41:0.99:1.00:0.09:0.16), (Janus.kinase.2.mutation:4:4840:0: 41:0.99:1.00:0.09:0.16), (Laparoscopic.surgery:4:4840:0:41:0.99:1.00:0.09:0.16), (Laryngeal.canc er.recurrent:4:4840:0:41:0.99:1.00:0.09:0.16), (Infective.myositis:3:4841:0:41:0.99:1.00:0.07:0.1 3), (Cerebellar.haemorrhage:2:4842:0:41:0.99:1.00:0.05:0.09), (Gastric.ulcer.perforation:2:4842:0: 41:0.99:1.00:0.05:0.09), (Haemorrhoid.operation:2:4842:0:41:0.99:1.00:0.05:0.09), (Joint.ankylo sis:2:4842:0:41:0.99:1.00:0.05:0.09), (Muscle.mass:2:4842:0:41:0.99:1.00:0.05:0.09), (Ageusia:1: 4843:0:41:0.99:1.00:0.02:0.05), (Antisocial.behaviour:1:4843:0:41:0.99:1.00:0.02:0.05), (Arterial. rupture:1:4843:0:41:0.99:1.00:0.02:0.05), (Metastases.to.the.respiratory.system:1:4843:0:41:0.99: 1.00:0.02:0.05), (Retinopathy.hypertensive:1:4843:0:41:0.99:1.00:0.02:0.05), (Sinobronchitis:1:4 843:0:41:0.99:1.00:0.02:0.05), (Cardiovascular.examination:0:4843:0:42:0.99:#NUM!:0.00:#NU M!), (Legal.problem:0:4843:0:42:0.99:#NUM!:0.00:#NUM!), (Trace.element.deficiency:0:4843:0: 42:0.99:#NUM!:0.00:#NUM!), (Umbilical.hernia:0:4843:0:42:0.99:#NUM!:0.00:#NUM!), (Hepa titis.B.reactivation:21:4822:0:42:0.99:1.00:0.33:0.50), (Hodgkin's.disease.recurrent:21:4822:0:42: 0.99:1.00:0.33:0.50), (Acute.hepatic.failure:15:4828:0:42:0.99:1.00:0.26:0.42), (Renal.and.liver.t ransplant:12:4831:0:42:0.99:1.00:0.22:0.36), (Pulmonary.toxicity:10:4833:0:42:0.99:1.00:0.19:0. 32), (Alveolitis:8:4835:0:42:0.99:1.00:0.16:0.28), (Cardioplegia:8:4835:0:42:0.99:1.00:0.16:0.28), (Thrombophlebitis.migrans:8:4835:0:42:0.99:1.00:0.16:0.28), (Large.intestinal.haemorrhage:7:48 36:0:42:0.99:1.00:0.14:0.25), (Stress.fracture:7:4836:0:42:0.99:1.00:0.14:0.25), (Urethral.cancer:7: 4836:0:42:0.99:1.00:0.14:0.25), (Blood.cortisol:6:4837:0:42:0.99:1.00:0.13:0.22), (Transitional.c ell.carcinoma.urethra:6:4837:0:42:0.99:1.00:0.13:0.22), (Cerebrovascular.insufficiency:5:4838:0: 42:0.99:1.00:0.11:0.19), (Hyperinsulinaemia:5:4838:0:42:0.99:1.00:0.11:0.19), (Atrioventricular.b lock.second.degree:4:4839:0:42:0.99:1.00:0.09:0.16), (Histiocytosis:4:4839:0:42:0.99:1.00:0.09: 0.16), (Moraxella.infection:4:4839:0:42:0.99:1.00:0.09:0.16), (Postoperative.wound.complication: 4:4839:0:42:0.99:1.00:0.09:0.16), (Therapeutic.aspiration:4:4839:0:42:0.99:1.00:0.09:0.16), (Carc inoma.in.situ:3:4840:0:42:0.99:1.00:0.07:0.13), (Hypertensive.emergency:3:4840:0:42:0.99:1.00: 0.07:0.13), (Large.intestinal.ulcer:3:4840:0:42:0.99:1.00:0.07:0.13), (Localised.intraabdominal.flu id.collection:3:4840:0:42:0.99:1.00:0.07:0.13), (Metastases.to.thorax:3:4840:0:42:0.99:1.00:0.07: 0.13), (Scan.brain:3:4840:0:42:0.99:1.00:0.07:0.13), (Vaginal.inflammation:3:4840:0:42:0.99:1.0 0:0.07:0.13), (Blood.albumin.increased:2:4841:0:42:0.99:1.00:0.05:0.09), (Colonoscopy.abnormal: 2:4841:0:42:0.99:1.00:0.05:0.09), (Cortisol.deficiency:2:4841:0:42:0.99:1.00:0.05:0.09), (Lower. respiratory.tract.inflammation:2:4841:0:42:0.99:1.00:0.05:0.09), (Lymphatic.mapping:2:4841:0:4 2:0.99:1.00:0.05:0.09), (Poorly.differentiated.thyroid.carcinoma:2:4841:0:42:0.99:1.00:0.05:0.09), (Rosai-Dorfman.syndrome:2:4841:0:42:0.99:1.00:0.05:0.09), (Sepsis.neonatal:2:4841:0:42:0.99:1.00:0.0 5:0.09), (Toxic.nodular.goitre:2:4841:0:42:0.99:1.00:0.05:0.09), (Homicidal.ideation:1:4842:0:42: 0.99:1.00:0.02:0.05), (Phlebitis.superficial:1:4842:0:42:0.99:1.00:0.02:0.05), (Post.procedural.my ocardial.infarction:1:4842:0:42:0.99:1.00:0.02:0.05), (Solar.dermatitis:1:4842:0:42:0.99:1.00:0.0 2:0.05), (Thalamic.infarction:1:4842:0:42:0.99:1.00:0.02:0.05), (Thoracic.cavity.drainage:1:4842: 0:42:0.99:1.00:0.02:0.05), (Arteriovenous.fistula.thrombosis:0:4842:0:43:0.99:#NUM!:0.00:#NU M!), (Staphylococcal.abscess:0:4842:0:43:0.99:#NUM!:0.00:#NUM!), (Epstein-Barr.virus.associated.lymphoma:19:4823:0:43:0.99:1.00:0.31:0.47), (Germ.cell.neoplasm:16:482 6:0:43:0.99:1.00:0.27:0.43), (Lepromatous.leprosy:16:4826:0:43:0.99:1.00:0.27:0.43), (Burkitt's.l ymphoma.stage.IV:15:4827:0:43:0.99:1.00:0.26:0.41), (Testicular.germ.cell.tumour:14:4828:0:4 3:0.99:1.00:0.25:0.39), (Disseminated.cryptococcosis:10:4832:0:43:0.99:1.00:0.19:0.32), (High.gr ade.B-cell.lymphoma.Burkitt-like.lymphoma.recurrent:10:4832:0:43:0.99:1.00:0.19:0.32), (Ocular.vascular.disorder:10:4832:0: 43:0.99:1.00:0.19:0.32), (Axillary.vein.thrombosis:8:4834:0:43:0.99:1.00:0.16:0.27), (Metapneum ovirus.infection:8:4834:0:43:0.99:1.00:0.16:0.27), (Aspergillus.test.positive:7:4835:0:43:0.99:1.0 0:0.14:0.25), (Body.temperature.fluctuation:7:4835:0:43:0.99:1.00:0.14:0.25), (Gastric.neoplasm: 7:4835:0:43:0.99:1.00:0.14:0.25), (Herpes.oesophagitis:7:4835:0:43:0.99:1.00:0.14:0.25), (Immat ure.respiratory.system:7:4835:0:43:0.99:1.00:0.14:0.25), (Intra-uterine.contraceptive.device:7:4835:0:43:0.99:1.00:0.14:0.25), (Bronchostenosis:5:4837:0:43:0.9 9:1.00:0.10:0.19), (Peau.d'orange:5:4837:0:43:0.99:1.00:0.10:0.19), (Psychiatric.decompensation: 5:4837:0:43:0.99:1.00:0.10:0.19), (Stent.removal:5:4837:0:43:0.99:1.00:0.10:0.19), (Antibiotic.le vel:4:4838:0:43:0.99:1.00:0.09:0.16), (Eosinophil.count.increased:4:4838:0:43:0.99:1.00:0.09:0.1 6), (Prostatic.dysplasia:4:4838:0:43:0.99:1.00:0.09:0.16), (Abscess.rupture:3:4839:0:43:0.99:1.00: 0.07:0.12), (Asthma.late.onset:3:4839:0:43:0.99:1.00:0.07:0.12), (Atrial.septal.defect.repair:3:483 9:0:43:0.99:1.00:0.07:0.12), (Cardiovascular.symptom:3:4839:0:43:0.99:1.00:0.07:0.12), (Hepatic.vein.occlusion:3:4839:0:43:0.99:1.00:0.07:0.12), (Hyperinsulinism:3:4839:0:43:0.99:1.00:0.07:0 0.12), (Hypoxic-ischaemic.encephalopathy:3:4839:0:43:0.99:1.00:0.07:0.12), (Sensitisation:3:4839:0:43:0.99:1.00: 0.07:0.12), (Chemical.injury:2:4840:0:43:0.99:1.00:0.04:0.09), (Dizziness.postural:2:4840:0:43:0 0.99:1.00:0.04:0.09), (Drug.prescribing.error:2:4840:0:43:0.99:1.00:0.04:0.09), (Enzyme.level.decr eased:2:4840:0:43:0.99:1.00:0.04:0.09), (Gastrointestinal.oedema:2:4840:0:43:0.99:1.00:0.04:0.0 9), (Homeopathy:2:4840:0:43:0.99:1.00:0.04:0.09), (Pelvic.discomfort:2:4840:0:43:0.99:1.00:0.0 4:0.09), (Reduction.of.increased.intracranial.pressure:2:4840:0:43:0.99:1.00:0.04:0.09), (Skin.frag ility:2:4840:0:43:0.99:1.00:0.04:0.09), (Systemic.infection:2:4840:0:43:0.99:1.00:0.04:0.09), (Alv eolar.osteitis:1:4841:0:43:0.99:1.00:0.02:0.04), (Apnoea.neonatal:1:4841:0:43:0.99:1.00:0.02:0.0 4), (Diabetic.metabolic.decompensation:1:4841:0:43:0.99:1.00:0.02:0.04), (Endometrial.neoplasm: 1:4841:0:43:0.99:1.00:0.02:0.04), (Infusion.site.reaction:1:4841:0:43:0.99:1.00:0.02:0.04), (Opht halmologic.treatment:1:4841:0:43:0.99:1.00:0.02:0.04), (Sternal.fracture:1:4841:0:43:0.99:1.00:0 0.02:0.04), (Abdomen.scan:0:4841:0:44:0.99:#NUM!:0.00:#NUM!), (Candida.osteomyelitis:0:484 1:0:44:0.99:#NUM!:0.00:#NUM!), (Hepatic.amoebiasis:0:4841:0:44:0.99:#NUM!:0.00:#NUM!), (Joint.dislocation.reduction:0:4841:0:44:0.99:#NUM!:0.00:#NUM!), (Neuromuscular.pain:0:484 1:0:44:0.99:#NUM!:0.00:#NUM!), (Pancreatitis.relapsing:0:4841:0:44:0.99:#NUM!:0.00:#NUM !), (Neuroblastoma.recurrent:16:4825:0:44:0.99:1.00:0.27:0.42), (Diffuse.large.B-cell.lymphoma.stage.III:15:4826:0:44:0.99:1.00:0.25:0.41), (Limbic.encephalitis:11:4830:0:44:0. 99:1.00:0.20:0.33), (Urinary.tract.infection.enterococcal:9:4832:0:44:0.99:1.00:0.17:0.29), (Absce ss.intestinal:8:4833:0:44:0.99:1.00:0.15:0.27), (Melanoma.recurrent:8:4833:0:44:0.99:1.00:0.15:0 0.27), (Antinuclear.antibody:7:4834:0:44:0.99:1.00:0.14:0.24), (Blood.cortisol.abnormal:7:4834:0: 44:0.99:1.00:0.14:0.24), (Blood.sodium.increased:7:4834:0:44:0.99:1.00:0.14:0.24), (Bone.densit y.increased:7:4834:0:44:0.99:1.00:0.14:0.24), (Deafness.neurosensory:7:4834:0:44:0.99:1.00:0.1 4:0.24), (Fundoscopy:7:4834:0:44:0.99:1.00:0.14:0.24), (Peripheral.sensorimotor.neuropathy:7:48 34:0:44:0.99:1.00:0.14:0.24), (Leukocyturia:6:4835:0:44:0.99:1.00:0.12:0.21), (Meningococcal.in fection:6:4835:0:44:0.99:1.00:0.12:0.21), (Reversal.of.sedation:6:4835:0:44:0.99:1.00:0.12:0.21), (Blindness.unilateral:5:4836:0:44:0.99:1.00:0.10:0.19), (Eclampsia:5:4836:0:44:0.99:1.00:0.10:0. 19), (Fungal.endocarditis:5:4836:0:44:0.99:1.00:0.10:0.19), (Procedural.complication:5:4836:0:44: 0.99:1.00:0.10:0.19), (Bone.atrophy:4:4837:0:44:0.99:1.00:0.08:0.15), (Deep.brain.stimulation:4: 4837:0:44:0.99:1.00:0.08:0.15), (Fibrous.dysplasia.of.bone:4:4837:0:44:0.99:1.00:0.08:0.15), (Me tastases.to.soft.tissue:4:4837:0:44:0.99:1.00:0.08:0.15), (Occupational.asthma:4:4837:0:44:0.99:1 0.00:0.08:0.15), (Wound.dehiscence:4:4837:0:44:0.99:1.00:0.08:0.15), (Aortic.bypass:3:4838:0:44: 0.99:1.00:0.06:0.12), (Diaphragmatic.disorder:3:4838:0:44:0.99:1.00:0.06:0.12), (Dysaesthesia.ph arynx:3:4838:0:44:0.99:1.00:0.06:0.12), (Extracorporeal.membrane.oxygenation:3:4838:0:44:0.9 9:1.00:0.06:0.12), (Hand.deformity:3:4838:0:44:0.99:1.00:0.06:0.12), (Intention.tremor:3:4838:0: 44:0.99:1.00:0.06:0.12), (Metabolic.abnormality.management:3:4838:0:44:0.99:1.00:0.06:0.12), (Myxoid.liposarcoma:3:4838:0:44:0.99:1.00:0.06:0.12), (Shunt.thrombosis:3:4838:0:44:0.99:1.00: 0.06:0.12), (Snoring:3:4838:0:44:0.99:1.00:0.06:0.12), (Staphylococcus.test:3:4838:0:44:0.99:1.0 0:0.06:0.12), (Thrombophlebitis.septic:3:4838:0:44:0.99:1.00:0.06:0.12), (Basal.cell.naevus.syndr ome:2:4839:0:44:0.99:1.00:0.04:0.08), (Coronary.artery.embolism:2:4839:0:44:0.99:1.00:0.04:0. 08), (Foreign.body.sensation.in.eyes:2:4839:0:44:0.99:1.00:0.04:0.08), (Implant.site.reaction:2:48 39:0:44:0.99:1.00:0.04:0.08), (Pseudomonas.bronchitis:2:4839:0:44:0.99:1.00:0.04:0.08), (Splenic.vein.thrombosis:2:4839:0:44:0.99:1.00:0.04:0.08), (Staphylococcal.osteomyelitis:2:4839:0:44:0. 99:1.00:0.04:0.08), (Progesterone:1:4840:0:44:0.99:1.00:0.02:0.04), (Pruritus.genital:1:4840:0:44: 0.99:1.00:0.02:0.04), (Skin.hypertrophy:1:4840:0:44:0.99:1.00:0.02:0.04), (Vascular.cauterisation: 1:4840:0:44:0.99:1.00:0.02:0.04), (Vocal.cord.paralysis:1:4840:0:44:0.99:1.00:0.02:0.04), (Distal.intestinal.obstruction.syndrome:0:4840:0:45:0.99:#NUM!:0.00:#NUM!), (Extraskeletal.chondros arcoma.metastatic:0:4840:0:45:0.99:#NUM!:0.00:#NUM!), (Puncture.site.pain:0:4840:0:45:0.99: #NUM!:0.00:#NUM!), (Viral.pharyngitis:0:4840:0:45:0.99:#NUM!:0.00:#NUM!), (Acute.HIV.in fection:26:4814:1:44:0.99:0.96:0.37:0.54), (Atypical.teratoid/rhabdoid.tumour.of.CNS:18:4822:0: 45:0.99:1.00:0.29:0.44), (Histoplasmosis.disseminated:16:4824:0:45:0.99:1.00:0.26:0.42), (Hodg kin's.disease.mixed.cellularity.stage.unspecified:16:4824:0:45:0.99:1.00:0.26:0.42), (Juvenile.my oclonic.epilepsy:15:4825:0:45:0.99:1.00:0.25:0.40), (Chromosome.analysis.abnormal:9:4831:0:4 5:0.99:1.00:0.17:0.29), (Giardiasis:9:4831:0:45:0.99:1.00:0.17:0.29), (Macular.fibrosis:9:4831:0:4 5:0.99:1.00:0.17:0.29), (Hypercalciuria:8:4832:0:45:0.99:1.00:0.15:0.26), (Hyperthermia.maligna nt:7:4833:0:45:0.99:1.00:0.13:0.24), (Antineutrophil.cytoplasmic.antibody.positive:6:4834:0:45:0 0.99:1.00:0.12:0.21), (Necrotising.scleritis:6:4834:0:45:0.99:1.00:0.12:0.21), (Campylobacter.infec tion:5:4835:0:45:0.99:1.00:0.10:0.18), (Porokeratosis:5:4835:0:45:0.99:1.00:0.10:0.18), (Tendon. pain:5:4835:0:45:0.99:1.00:0.10:0.18), (Adiposis.dolorosa:4:4836:0:45:0.99:1.00:0.08:0.15), (Cat aract.operation.complication:4:4836:0:45:0.99:1.00:0.08:0.15), (Oral.discharge:4:4836:0:45:0.99: 1.00:0.08:0.15), (Alcoholic:3:4837:0:45:0.99:1.00:0.06:0.12), (CNS.ventriculitis:3:4837:0:45:0.99: 1.00:0.06:0.12), (Lobular.breast.carcinoma.in.situ:3:4837:0:45:0.99:1.00:0.06:0.12), (Prostatitis.E scherichia.coli:3:4837:0:45:0.99:1.00:0.06:0.12), (Duodenal.ulcer.haemorrhage:2:4838:0:45:0.99: 1.00:0.04:0.08), (Hepatectomy:2:4838:0:45:0.99:1.00:0.04:0.08), (Metabolic.alkalosis:2:4838:0:4 5:0.99:1.00:0.04:0.08), (Mixed.incontinence:2:4838:0:45:0.99:1.00:0.04:0.08), (Neurofibroma:2:4 838:0:45:0.99:1.00:0.04:0.08), (Perineal.erythema:2:4838:0:45:0.99:1.00:0.04:0.08), (Pubis.fractu re:2:4838:0:45:0.99:1.00:0.04:0.08), (Cardiopulmonary.failure:0:4839:0:46:0.99:#NUM!:0.00:#N UM!), (Conjunctival.disorder:0:4839:0:46:0.99:#NUM!:0.00:#NUM!), (Drug.diversion:0:4839:0: 46:0.99:#NUM!:0.00:#NUM!), (Enzyme.activity.decreased:0:4839:0:46:0.99:#NUM!:0.00:#NU M!), (Polycystic.liver.disease:0:4839:0:46:0.99:#NUM!:0.00:#NUM!), (Thyroid.cancer.stage.IV:0: 4839:0:46:0.99:#NUM!:0.00:#NUM!), (Rheumatoid.factor:18:4821:0:46:0.99:1.00:0.28:0.44), (Graft.complication:10:4829:0:46:0.99:1.00:0.18:0.30), (Chordoma:9:4830:0:46:0.99:1.00:0.16:0. 28), (Mycobacterium.chelonae.infection:9:4830:0:46:0.99:1.00:0.16:0.28), (Tuberculosis.of.central. nervous.system:8:4831:0:46:0.99:1.00:0.15:0.26), (Hypouricaemia:7:4832:0:46:0.99:1.00:0.13: 0.23), (Feeding.intolerance:6:4833:0:46:0.99:1.00:0.12:0.21), (Retinitis:6:4833:0:46:0.99:1.00:0.1 2:0.21), (Skin.test.positive:6:4833:0:46:0.99:1.00:0.12:0.21), (Blastomycosis:5:4834:0:46:0.99:1.0 0:0.10:0.18), (Factor.II.mutation:5:4834:0:46:0.99:1.00:0.10:0.18), (Congenital.hypothyroidism:4: 4835:0:46:0.99:1.00:0.08:0.15), (Hepatitis.alcoholic:4:4835:0:46:0.99:1.00:0.08:0.15), (Injection.s ite.rash:4:4835:0:46:0.99:1.00:0.08:0.15), (Menstrual.discomfort:4:4835:0:46:0.99:1.00:0.08:0.15), (Myelodysplastic.syndrome.transformation:4:4835:0:46:0.99:1.00:0.08:0.15), (Septic.embolus:4: 4835:0:46:0.99:1.00:0.08:0.15), (Vitamin.B1:4:4835:0:46:0.99:1.00:0.08:0.15), (Crepitations:3:4 836:0:46:0.99:1.00:0.06:0.12), (Histamine.level.increased:3:4836:0:46:0.99:1.00:0.06:0.12), (Res piratory.rate:3:4836:0:46:0.99:1.00:0.06:0.12), (Subclavian.vein.thrombosis:3:4836:0:46:0.99:1.0 0:0.06:0.12), (Abdominal.cavity.drainage:2:4837:0:46:0.99:1.00:0.04:0.08), (Catheter.removal:2:4 837:0:46:0.99:1.00:0.04:0.08), (Chondritis:2:4837:0:46:0.99:1.00:0.04:0.08), (Disuse.syndrome:2: 4837:0:46:0.99:1.00:0.04:0.08), (Klebsiella.sepsis:2:4837:0:46:0.99:1.00:0.04:0.08), (Nocturnal. d yspnoea:2:4837:0:46:0.99:1.00:0.04:0.08), (Typhoid.fever:2:4837:0:46:0.99:1.00:0.04:0.08), (Vas cular.compression:2:4837:0:46:0.99:1.00:0.04:0.08), (Hereditary.neuropathic.amyloidosis:1:4838: 0:46:0.99:1.00:0.02:0.04), (Hypothyroidic.goitre:1:4838:0:46:0.99:1.00:0.02:0.04), (Legionella.in fection:1:4838:0:46:0.99:1.00:0.02:0.04), (Pancreas.infection:1:4838:0:46:0.99:1.00:0.02:0.04), (P urging:1:4838:0:46:0.99:1.00:0.02:0.04), (Arrhythmia.neonatal:0:4838:0:47:0.99:#NUM!:0.00:#NUM!), (Lung.lobectomy:0:4838:0:47:0.99:#NUM!:0.00:#NUM!), (Rheumatoid.lung:15:4823:0: 47:0.99:1.00:0.24:0.39), (Metastases.to.retroperitoneum:12:4826:0:47:0.99:1.00:0.20:0.34), (Cyto kine.storm:10:4828:0:47:0.99:1.00:0.18:0.30), (Encephalitis.cytomegalovirus:10:4828:0:47:0.99: 1.00:0.18:0.30), (BRCA2.gene.mutation:9:4829:0:47:0.99:1.00:0.16:0.28), (Hepatitis.cholestatic:9: 4829:0:47:0.99:1.00:0.16:0.28), (Liposarcoma.metastatic:9:4829:0:47:0.99:1.00:0.16:0.28), (Oste ochondrosis:9:4829:0:47:0.99:1.00:0.16:0.28), (Asplenia:8:4830:0:47:0.99:1.00:0.15:0.25), (Cory nebacterium.infection:8:4830:0:47:0.99:1.00:0.15:0.25), (Mycobacterium.fortuitum.infection:8:4 830:0:47:0.99:1.00:0.15:0.25), (Anaemia.megaloblastic:7:4831:0:47:0.99:1.00:0.13:0.23), (Hypop noea:7:4831:0:47:0.99:1.00:0.13:0.23), (Pneumonia.3), (Squamous.cell.carcinoma.of.the.cervix:7:4831:0:47:0.99:1.00:0.13:0.23), (Formication:6:483 2:0:47:0.99:1.00:0.11:0.20), (Onychomadesis:6:4832:0:47:0.99:1.00:0.11:0.20), (Primary.insulin.I ike.growth.factor-1.deficiency:6:4832:0:47:0.99:1.00:0.11:0.20), (Prostatic.specific.antigen.abnormal:6:4832:0:47: 0.99:1.00:0.11:0.20), (Cystitis.bacterial:5:4833:0:47:0.99:1.00:0.10:0.18), (Light.anaesthesia:5:48 33:0:47:0.99:1.00:0.10:0.18), (Bacteriuria:4:4834:0:47:0.99:1.00:0.08:0.15), (Breast.mass:4:4834: 0:47:0.99:1.00:0.08:0.15), (Metastases.to.large.intestine:4:4834:0:47:0.99:1.00:0.08:0.15), (Candi da.endophthalmitis:3:4835:0:47:0.99:1.00:0.06:0.11), (Facial.bones.fracture:3:4835:0:47:0.99:1.0 0:0.06:0.11), (Gastritis.bacterial:3:4835:0:47:0.99:1.00:0.06:0.11), (Hyperinsulinaemic.hypoglyca emia:3:4835:0:47:0.99:1.00:0.06:0.11), (Klebsiella.bacteraemia:3:4835:0:47:0.99:1.00:0.06:0.11), (Menopausal.depression:3:4835:0:47:0.99:1.00:0.06:0.11), (Superinfection.bacterial:3:4835:0:47: 0.99:1.00:0.06:0.11), (Tarsal.tunnel.syndrome:3:4835:0:47:0.99:1.00:0.06:0.11), (Tonic.clonic.m ovements:3:4835:0:47:0.99:1.00:0.06:0.11), (Anaemia.neonatal:2:4836:0:47:0.99:1.00:0.04:0.08), (Bladder.operation:2:4836:0:47:0.99:1.00:0.04:0.08), (Eye.infection.fungal:2:4836:0:47:0.99:1.0 0:0.04:0.08), (Nutritional.condition.abnormal:2:4836:0:47:0.99:1.00:0.04:0.08), (Salivary.gland.n eoplasm:2:4836:0:47:0.99:1.00:0.04:0.08), (Breast.dysplasia:1:4837:0:47:0.99:1.00:0.02:0.04), (C olectomy.total:1:4837:0:47:0.99:1.00:0.02:0.04), (Glucocorticoid.deficiency:1:4837:0:47:0.99:1.0 0:0.02:0.04), (Lower.respiratory.tract.infection.bacterial:1:4837:0:47:0.99:1.00:0.02:0.04), (Ortho dontic.procedure:1:4837:0:47:0.99:1.00:0.02:0.04), (Tethered.cord.syndrome:1:4837:0:47:0.99:1. 00:0.02:0.04), (Laparotomy:0:4837:0:48:0.99:#NUM!:0.00:#NUM!), (Oesophageal.hypomotility: 0:4837:0:48:0.99:#NUM!:0.00:#NUM!), (Self-injurious.ideation:0:4837:0:48:0.99:#NUM!:0.00:#NUM!), (Metastases.to.pancreas:17:4820:0:48: 0.99:1.00:0.26:0.41), (Polyomavirus-associated.nephropathy:15:4822:0:48:0.99:1.00:0.24:0.38), (Chronic.myeloid.leukaemia.recurrent: 14:4823:0:48:0.99:1.00:0.23:0.37), (Cutaneous.vasculitis:11:4826:0:48:0.99:1.00:0.19:0.31), (Ne utropenic.infection:11:4826:0:48:0.99:1.00:0.19:0.31), (Medulloblastoma.recurrent:10:4827:0:48: 0.99:1.00:0.17:0.29), (Necrotising.retinitis:9:4828:1:47:0.99:0.90:0.16:0.27), (Prolymphocytic.leu kaemia:8:4829:0:48:0.99:1.00:0.14:0.25), (Cor.pulmonale.acute:7:4830:0:48:0.99:1.00:0.13:0.23), (Gastrointestinal.cancer.metastatic:7:4830:0:48:0.99:1.00:0.13:0.23), (Nasal.ulcer:7:4830:0:48:0. 99:1.00:0.13:0.23), (Gastroenteritis.salmonella:6:4831:0:48:0.99:1.00:0.11:0.20), (Trichosporon.i nfection:6:4831:0:48:0.99:1.00:0.11:0.20), (Urate.nephropathy:6:4831:0:48:0.99:1.00:0.11:0.20), (Ankle.operation:5:4832:0:48:0.99:1.00:0.09:0.17), (Benign.renal.neoplasm:5:4832:0:48:0.99:1.0 0:0.09:0.17), (Eye.degenerative.disorder:5:4832:0:48:0.99:1.00:0.09:0.17), (Hyperadrenalism:5:4 832:0:48:0.99:1.00:0.09:0.17), (Perforated.ulcer:5:4832:0:48:0.99:1.00:0.09:0.17), (Pneumonia.ne crotising:5:4832:0:48:0.99:1.00:0.09:0.17), (Rett.syndrome:5:4832:0:48:0.99:1.00:0.09:0.17), (Tr ansaminases.decreased:5:4832:0:48:0.99:1.00:0.09:0.17), (Benign.neoplasm.of.adrenal.gland:4:4 833:0:48:0.99:1.00:0.08:0.14), (Enthesopathy:4:4833:0:48:0.99:1.00:0.08:0.14), (Lithotripsy:4:48 33:0:48:0.99:1.00:0.08:0.14), (Microembolism:4:4833:0:48:0.99:1.00:0.08:0.14), (Subileus:4:483 3:0:48:0.99:1.00:0.08:0.14), (Uterine.atony:4:4833:0:48:0.99:1.00:0.08:0.14), (Hereditary.spheroc ytosis:3:4834:0:48:0.99:1.00:0.06:0.11), (Ocular.toxicity:3:4834:0:48:0.99:1.00:0.06:0.11), (Post. procedural.diarrhoea:3:4834:0:48:0.99:1.00:0.06:0.11), (Urethral.pain:3:4834:0:48:0.99:1.00:0.06:0.11), (Ventricular.flutter:3:4834:0:48:0.99:1.00:0.06:0.11), (Wound.infection.34:0:48:0.99:1.00:0.06:0.11), (Breast.cellulitis:2:4835:0:48:0.99:1.00:0.04:0.08), (Cholesterosis:2: 4835:0:48:0.99:1.00:0.04:0.08), (Facial.nerve.disorder:2:4835:0:48:0.99:1.00:0.04:0.08), (Gastroi ntestinal.perforation:2:4835:0:48:0.99:1.00:0.04:0.08), (Lymph.node.pain:2:4835:0:48:0.99:1.00: 0.04:0.08), (Osteoarthropathy:2:4835:0:48:0.99:1.00:0.04:0.08), (Rectal.spasm:2:4835:0:48:0.99: 1.00:0.04:0.08), (Vulvovaginal.disorder:2:4835:0:48:0.99:1.00:0.04:0.08), (Eyelids.pruritus:1:483 6:0:48:0.99:1.00:0.02:0.04), (Frostbite:1:4836:0:48:0.99:1.00:0.02:0.04), (Logorrhoea:1:4836:0:4 8:0.99:1.00:0.02:0.04), (Medical.device.site.infection:1:4836:0:48:0.99:1.00:0.02:0.04), (Meige's. syndrome:1:4836:0:48:0.99:1.00:0.02:0.04), (Steatohepatitis:1:4836:0:48:0.99:1.00:0.02:0.04), (T orsade.de.pointes:0:4836:0:49:0.99:#NUM!:0.00:#NUM!), (Germ.cell.cancer:22:4814:0:49:0.99: 1.00:0.31:0.47), (Primary.mediastinal.large.B-cell.lymphoma:22:4814:0:49:0.99:1.00:0.31:0.47), (Immunosuppressant.drug.level:18:4818:0:49: 0.99:1.00:0.27:0.42), (Metastases.to.stomach:13:4823:0:49:0.99:1.00:0.21:0.35), (Aortitis:11:482 5:0:49:0.99:1.00:0.18:0.31), (Ichthyosis:11:4825:0:49:0.99:1.00:0.18:0.31), (Philadelphia.chromo some.negative:11:4825:0:49:0.99:1.00:0.18:0.31), (Contraceptive.cap:9:4827:0:49:0.99:1.00:0.16: 0.27), (Post.lumbar.puncture.syndrome:9:4827:0:49:0.99:1.00:0.16:0.27), (Right.ventricular.dysf unction:9:4827:0:49:0.99:1.00:0.16:0.27), (Whipple's.disease:9:4827:0:49:0.99:1.00:0.16:0.27), (Herpes.dermatitis:8:4828:0:49:0.99:1.00:0.14:0.25), (Quadriparesis:7:4829:0:49:0.99:1.00:0.13:0 0.22), (Allergy.to.arthropod.bite:6:4830:0:49:0.99:1.00:0.11:0.20), (Alpha.haemolytic.streptococcal.infection:6:4830:0:49:0.99:1.00:0.11:0.20), (Breast.tenderness:6:4830:0:49:0.99:1.00:0.11:0.20), (Gallbladder.cancer.metastatic:6:4830:0:49:0.99:1.00:0.11:0.20), (Systolic.hypertension:6:4830:0: 49:0.99:1.00:0.11:0.20), (Diaphragmatic.paralysis:5:4831:0:49:0.99:1.00:0.09:0.17), (Radiothera py.to.thyroid:5:4831:0:49:0.99:1.00:0.09:0.17), (Walking.disability:5:4831:0:49:0.99:1.00:0.09:0. 17), (Aspirin-exacerbated.respiratory.disease:4:4832:0:49:0.99:1.00:0.08:0.14), (Delayed.puberty:4:4832:0:49: 0.99:1.00:0.08:0.14), (Hypertrophy:4:4832:0:49:0.99:1.00:0.08:0.14), (Vascular.pseudoaneurysm: 4:4832:0:49:0.99:1.00:0.08:0.14), (Actinomycosis:3:4833:0:49:0.99:1.00:0.06:0.11), (Blood.test:3: 4833:0:49:0.99:1.00:0.06:0.11), (Colon.operation:3:4833:0:49:0.99:1.00:0.06:0.11), (Facet.joint.s yndrome:3:4833:0:49:0.99:1.00:0.06:0.11), (Fungal.abscess.central.nervous.system:3:4833:0:49: 0.99:1.00:0.06:0.11), (Oesophageal.discomfort:3:4833:0:49:0.99:1.00:0.06:0.11), (Balanoposthitis: 2:4834:0:49:0.99:1.00:0.04:0.08), (Blood.electrolytes.decreased:2:4834:0:49:0.99:1.00:0.04:0.08), (Dyskinesia.oesophageal:2:4834:0:49:0.99:1.00:0.04:0.08), (Metastases.to.gastrointestinal.tract: 2:4834:0:49:0.99:1.00:0.04:0.08), (Oropharyngeal.plaque:2:4834:0:49:0.99:1.00:0.04:0.08), (Rem oval.of.inert.matter.from.skin.or.subcutaneous.tissue:2:4834:0:49:0.99:1.00:0.04:0.08), (Renal.cell. carcinoma.recurrent:2:4834:0:49:0.99:1.00:0.04:0.08), (Cervicogenic.headache:1:4835:0:49:0.9 9:1.00:0.02:0.04), (Eye.bums:1:4835:0:49:0.99:1.00:0.02:0.04), (Inadequate.diet:1:4835:0:49:0.99: 1.00:0.02:0.04), (Nodal.arrhythmia:1:4835:0:49:0.99:1.00:0.02:0.04), (Root.canal.infection:1:483 5:0:49:0.99:1.00:0.02:0.04), (Selective.polysaccharide.antibody.deficiency:1:4835:0:49:0.99:1.00: 0.02:0.04), (Hypertension.neonatal:0:4835:0:50:0.99:#NUM!:0.00:#NUM!), (Interventional.proc edure:0:4835:0:50:0.99:#NUM!:0.00:#NUM!), (Small.intestinal.haemorrhage:0:4835:0:50:0.99:#NUM!:0.00:#NUM!), (Streptococcal.endocarditis:0:4835:0:50:0.99:#NUM!:0.00:#NUM!), (Recto sigmoid.cancer.metastatic:16:4819:0:50:0.99:1.00:0.24:0.39), (Natural.killer-cell.lymphoblastic.lymphoma:15:4820:0:50:0.99:1.00:0.23:0.38), (Pneumonia.streptococcal:10:4 825:0:50:0.99:1.00:0.17:0.29), (Brucellosis:9:4826:0:50:0.99:1.00:0.15:0.26), (Exposure.via.body. fluid:9:4826:0:50:0.99:1.00:0.15:0.26), (Hypocalciuria:9:4826:0:50:0.99:1.00:0.15:0.26), (Pharyn geal.cancer:9:4826:0:50:0.99:1.00:0.15:0.26), (Pulmonary.granuloma:9:4826:0:50:0.99:1.00:0.15: 0.26), (Adrenogenital.syndrome:8:4827:0:50:0.99:1.00:0.14:0.24), (Bronchial.neoplasm:8:4827:0:50:0.99:1.00:0.14:0.24), (Eczema.infected:8:4827:0:50:0.99:1.00:0.14:0.24), (Unrelated.donor.bo ne.marrow.transplantation.therapy:8:4827:0:50:0.99:1.00:0.14:0.24), (Ear.swelling:6:4829:0:50:0 0.99:1.00:0.11:0.19), (Genital.infection.fungal:6:4829:0:50:0.99:1.00:0.11:0.19), (Large.cell.lung.c ancer.metastatic:6:4829:0:50:0.99:1.00:0.11:0.19), (Gastric.ulcer.helicobacter:5:4830:0:50:0.99:1 0.00:0.09:0.17), (Gastric.varices:5:4830:0:50:0.99:1.00:0.09:0.17), (Groin.infection:5:4830:0:50:0. 99:1.00:0.09:0.17), (Intervertebral.disc.injury:5:4830:0:50:0.99:1.00:0.09:0.17), (Oesophageal.inf ection:5:4830:0:50:0.99:1.00:0.09:0.17), (Rash.follicular:5:4830:0:50:0.99:1.00:0.09:0.17), (Uteri ne.contractions.abnormal:5:4830:0:50:0.99:1.00:0.09:0.17), (Benign.neoplasm:4:4831:0:50:0.99: 1.00:0.07:0.14), (Dehydroepiandrosterone.decreased:4:4831:0:50:0.99:1.00:0.07:0.14), (Dementia.of.the.Alzheimer's.type,.uncomplicated:4:4831:0:50:0.99:1.00:0.07:0.14), (Hypergammaglobulin aemia:4:4831:0:50:0.99:1.00:0.07:0.14), (Large.intestine.perforation:4:4831:0:50:0.99:1.00:0.07: 0.14), (Lung.adenocarcinoma.stage.II:4:4831:0:50:0.99:1.00:0.07:0.14), (Urinary.tract.inflammati on:4:4831:0:50:0.99:1.00:0.07:0.14), (Catheter.site.erythema:3:4832:0:50:0.99:1.00:0.06:0.11), (I ntestinal.sepsis:3:4832:0:50:0.99:1.00:0.06:0.11), (Multiple.organ.dysfunction.syndrome:3:4832: 0:50:0.99:1.00:0.06:0.11), (Progestin.replacement.therapy:3:4832:0:50:0.99:1.00:0.06:0.11), (Skin.necrosis:3:4832:0:50:0.99:1.00:0.06:0.11), (Bladder.outlet.obstruction:2:4833:0:50:0.99:1.00:0.0 4:0.07), (Blood.prolactin.abnormal:2:4833:0:50:0.99:1.00:0.04:0.07), (Illness.anxiety.disorder:2:4 833:0:50:0.99:1.00:0.04:0.07), (Penile.ulceration:2:4833:0:50:0.99:1.00:0.04:0.07), (Platelet.aggre gation.abnormal:2:4833:0:50:0.99:1.00:0.04:0.07), (Spina.bifida:2:4833:0:50:0.99:1.00:0.04:0.07), (Status.migrainosus:2:4833:0:50:0.99:1.00:0.04:0.07), (Thirst:2:4833:0:50:0.99:1.00:0.04:0.07), (Vulvovaginal.swelling:2:4833:0:50:0.99:1.00:0.04:0.07), (Amino.acid.level:1:4834:0:50:0.99:1. 00:0.02:0.04), (Dental.local.anaesthesia:1:4834:0:50:0.99:1.00:0.02:0.04), (Hair.injury:1:4834:0:5 0:0.99:1.00:0.02:0.04), (Vascular.encephalopathy:1:4834:0:50:0.99:1.00:0.02:0.04), (Bacillus.infe ction:0:4834:0:51:0.99:#NUM!:0.00:#NUM!), (Enterobiasis:0:4834:0:51:0.99:#NUM!:0.00:#NU M!), (Pudendal.canal.syndrome:0:4834:0:51:0.99:#NUM!:0.00:#NUM!), (Cytomegalovirus.enter ocolitis:12:4822:0:51:0.99:1.00:0.19:0.32), (Lupus.enteritis:12:4822:0:51:0.99:1.00:0.19:0.32), (V asculitis.cerebral:11:4823:0:51:0.99:1.00:0.18:0.30), (Peritoneal.tuberculosis:10:4824:0:51:0.99:1 0.00:0.16:0.28), (Scleroderma.renal.crisis:10:4824:0:51:0.99:1.00:0.16:0.28), (Autoimmune.neurop athy:9:4825:0:51:0.99:1.00:0.15:0.26), (Blood.growth.hormone.increased:9:4825:0:51:0.99:1.00: 0.15:0.26), (Collagen-vascular.disease:9:4825:0:51:0.99:1.00:0.15:0.26), (Adenoid.cystic.carcinoma:7:4827:0:51:0.99:1 0.00:0.12:0.22), (Cerebral.oedema.management:6:4828:0:51:0.99:1.00:0.11:0.19), (DiGeorge's.syn drome:6:4828:0:51:0.99:1.00:0.11:0.19), (Acute.promyelocytic.leukaemia.differentiation.syndro me:5:4829:0:51:0.99:1.00:0.09:0.16), (Clostridial.infection:5:4829:0:51:0.99:1.00:0.09:0.16), (Ski n.haemorrhage:5:4829:0:51:0.99:1.00:0.09:0.16), (Transitional.cell.carcinoma.metastatic:5:4829: 0:51:0.99:1.00:0.09:0.16), (Breast.conserving.surgery:4:4830:0:51:0.99:1.00:0.07:0.14), (Coagula tion.time.prolonged:4:4830:0:51:0.99:1.00:0.07:0.14), (Factor.VII.deficiency:4:4830:0:51:0.99:1. 00:0.07:0.14), (Glycosuria:4:4830:0:51:0.99:1.00:0.07:0.14), (Haemofiltration:4:4830:0:51:0.99:1 0.00:0.07:0.14), (Mycotic.endophthalmitis:4:4830:0:51:0.99:1.00:0.07:0.14), (Periorbital.oedema:4: 4830:0:51:0.99:1.00:0.07:0.14), (Biopsy.lymph.gland:3:4831:0:51:0.99:1.00:0.06:0.11), (Blood.b rain.barrier.defect:3:4831:0:51:0.99:1.00:0.06:0.11), (Dental.implantation:3:4831:0:51:0.99:1.00: 0.06:0.11), (Lipase.increased:3:4831:0:51:0.99:1.00:0.06:0.11), (Bacterial.colitis:1:4833:0:51:0.99: 1.00:0.02:0.04), (Bacterial.prostatitis:1:4833:0:51:0.99:1.00:0.02:0.04), (Blood.chloride.abnormal: 1:4833:0:51:0.99:1.00:0.02:0.04), (Negative.thoughts:1:4833:0:51:0.99:1.00:0.02:0.04), (Penis.di sorder:1:4833:0:51:0.99:1.00:0.02:0.04), (Pharyngotonsillitis:1:4833:0:51:0.99:1.00:0.02:0.04), (P rotein.total.abnormal:1:4833:0:51:0.99:1.00:0.02:0.04), (Tobacco.withdrawal.symptoms:1:4833:0: 51:0.99:1.00:0.02:0.04), (Lipoprotein.(a).increased:0:4833:0:52:0.99:#NUM!:0.00:#NUM!), (Pel vic.infection:0:4833:0:52:0.99:#NUM!:0.00:#NUM!), (Type.2.lepra.reaction:13:4820:0:52:0.99:1 0.00:0.20:0.33), (Fibrosarcoma:11:4822:0:52:0.99:1.00:0.17:0.30), (Subacute.cutaneous.lupus.eryt hematosus:11:4822:0:52:0.99:1.00:0.17:0.30), (Autoimmune.retinopathy:9:4824:0:52:0.99:1.00:0 0.15:0.26), (Peritoneal.carcinoma.metastatic:8:4825:0:52:0.99:1.00:0.13:0.24), (Transplant.failure: 8:4825:0:52:0.99:1.00:0.13:0.24), (Budd-Chiari.syndrome:7:4826:0:52:0.99:1.00:0.12:0.21), (Endocrine.ophthalmopathy:7:4826:0:52:0.99: 1.00:0.12:0.21), (Pneumonia.respiratory.syncytial.viral:7:4826:0:52:0.99:1.00:0.12:0.21), (Tenos ynovitis.stenosans:7:4826:0:52:0.99:1.00:0.12:0.21), (Autonomic.dysreflexia:6:4827:0:52:0.99:1. 00:0.10:0.19), (Coagulation.time:5:4828:0:52:0.99:1.00:0.09:0.16), (Pelvic.abscess:5:4828:0:52:0. 99:1.00:0.09:0.16), (Spinocerebellar.disorder:5:4828:0:52:0.99:1.00:0.09:0.16), (Malignant.nervo us.system.neoplasm:4:4829:0:52:0.99:1.00:0.07:0.13), (Multiple-drug.resistance:4:4829:0:52:0.99:1.00:0.07:0.13), (Vascular.stent.thrombosis:4:4829:0:52:0.99:1. 00:0.07:0.13), (Amylase.increased:3:4830:0:52:0.99:1.00:0.05:0.10), (Eye.laser.surgery:3:4830:0: 52:0.99:1.00:0.05:0.10), (Fatty.acid.deficiency:3:4830:0:52:0.99:1.00:0.05:0.10), (Food.poisoning: 3:4830:0:52:0.99:1.00:0.05:0.10), (Hypothermia:3:4830:0:52:0.99:1.00:0.05:0.10), (Nuclear.mag netic.resonance.imaging.spinal:3:4830:0:52:0.99:1.00:0.05:0.10), (Stupor:3:4830:0:52:0.99:1.00: 0.05:0.10), (Chronic.lymphocytic.leukaemia.stage.3:2:4831:0:52:0.99:1.00:0.04:0.07), (Hyperrefl exia:2:4831:0:52:0.99:1.00:0.04:0.07), (Postinfarction.angina:2:4831:0:52:0.99:1.00:0.04:0.07), (Single.photon.emission.computerised.tomogram:2:4831:0:52:0.99:1.00:0.04:0.07), (Tracheomala cia:2:4831:0:52:0.99:1.00:0.04:0.07), (Acquired.oesophageal.web:1:4832:0:52:0.99:1.00:0.02:0.0 4), (Arthroscopic.surgery:1:4832:0:52:0.99:1.00:0.02:0.04), (Biopsy.skin:1:4832:0:52:0.99:1.00:0. 02:0.04), (Heart.alternation:1:4832:0:52:0.99:1.00:0.02:0.04), (Ligament.disorder:1:4832:0:52:0.9 9:1.00:0.02:0.04), (Pseudohypoparathyroidism:1:4832:0:52:0.99:1.00:0.02:0.04), (Tracheostomy: 1:4832:0:52:0.99:1.00:0.02:0.04), (Gastrointestinal.angiodysplasia.haemorrhagic:0:4832:0:53:0.9 9:#NUM!:0.00:#NUM!), (Hypermobility.syndrome:0:4832:0:53:0.99:#NUM!:0.00:#NUM!), (Pri mary.hypercholesterolaemia:0:4832:0:53:0.99:#NUM!:0.00:#NUM!), (Hepatitis.B.virus.test.posit ive:16:4816:0:53:0.99:1.00:0.23:0.38), (Meningitis.staphylococcal:12:4820:0:53:0.99:1.00:0.18:0 0.31), (Oligoastrocytoma:12:4820:0:53:0.99:1.00:0.18:0.31), (Brain.stem.glioma:11:4821:0:53:0.9 9:1.00:0.17:0.29), (Cellulitis.orbital:11:4821:0:53:0.99:1.00:0.17:0.29), (Scrotal.ulcer:11:4821:0:5 3:0.99:1.00:0.17:0.29), (Cerebral.atrophy:9:4823:0:53:0.99:1.00:0.15:0.25), (Cerebral.aspergillosi s:8:4824:0:53:0.99:1.00:0.13:0.23), (Injection.site.swelling:8:4824:0:53:0.99:1.00:0.13:0.23), (Lis teriosis:8:4824:0:53:0.99:1.00:0.13:0.23), (Anal.ulcer:7:4825:0:53:0.99:1.00:0.12:0.21), (Enteroco litis.viral:7:4825:0:53:0.99:1.00:0.12:0.21), (Muscle.fatigue:7:4825:0:53:0.99:1.00:0.12:0.21), (Bi opsy.kidney:6:4826:0:53:0.99:1.00:0.10:0.18), (Maculopathy:6:4826:0:53:0.99:1.00:0.10:0.18), (P roteus.infection:6:4826:0:53:0.99:1.00:0.10:0.18), (Acid.peptic.disease:5:4827:0:53:0.99:1.00:0.0 9:0.16), (Anaplastic.oligodendroglioma:5:4827:0:53:0.99:1.00:0.09:0.16), (Arterial.spasm:5:4827: 0:53:0.99:1.00:0.09:0.16), (Blood.antidiuretic.hormone:5:4827:0:53:0.99:1.00:0.09:0.16), (Endom etritis:5:4827:0:53:0.99:1.00:0.09:0.16), (Enterobacter.sepsis:5:4827:0:53:0.99:1.00:0.09:0.16), (Gastric.dilatation:5:4827:0:53:0.99:1.00:0.09:0.16), (Gynaecomastia:5:4827:0:53:0.99:1.00:0.09: 0.16), (Haemorrhage.urinary.tract:5:4827:0:53:0.99:1.00:0.09:0.16), (Large.intestinal.obstruction: 5:4827:0:53:0.99:1.00:0.09:0.16), (Macule:5:4827:0:53:0.99:1.00:0.09:0.16), (Pharyngeal.inflam mation:5:4827:0:53:0.99:1.00:0.09:0.16), (Varicose.ulceration:4:4828:0:53:0.99:1.00:0.07:0.13), (Behavioural.therapy:3:4829:0:53:0.99:1.00:0.05:0.10), (Benign.neoplasm.of.prostate:3:4829:0:53: 0.99:1.00:0.05:0.10), (Chondrocalcinosis:3:4829:0:53:0.99:1.00:0.05:0.10), (Electroconvulsive.th erapy:3:4829:0:53:0.99:1.00:0.05:0.10), (Endometrial.cancer.metastatic:3:4829:0:53:0.99:1.00:0. 05:0.10), (Gastrointestinal.fungal.infection:3:4829:0:53:0.99:1.00:0.05:0.10), (Plaque.shift:3:4829: 0:53:0.99:1.00:0.05:0.10), (Q.fever:3:4829:0:53:0.99:1.00:0.05:0.10), (Respiratory.tract.oedema: 3:4829:0:53:0.99:1.00:0.05:0.10), (Blood.immunoglobulin.G:2:4830:0:53:0.99:1.00:0.04:0.07), (Bronchioloalveolar.carcinoma:2:4830:0:53:0.99:1.00:0.04:0.07), (Conversion.disorder:2:4830:0: 53:0.99:1.00:0.04:0.07), (Effusion:2:4830:0:53:0.99:1.00:0.04:0.07), (Nerve.root.compression:2:4 830:0:53:0.99:1.00:0.04:0.07), (Reactive.airways.dysfunction.syndrome:2:4830:0:53:0.99:1.00:0. 04:0.07), (Skin.atrophy:2:4830:0:53:0.99:1.00:0.04:0.07), (Borrelia.infection:1:4831:0:53:0.99:1.0 0:0.02:0.04), (Eczema.herpeticum:1:4831:0:53:0.99:1.00:0.02:0.04), (Hair.texture.abnormal:1:483 1:0:53:0.99:1.00:0.02:0.04), (Renal.cancer.recurrent:1:4831:0:53:0.99:1.00:0.02:0.04), (Spasmodi c.dysphonia:1:4831:0:53:0.99:1.00:0.02:0.04), (Oxygen.consumption.decreased:0:4831:0:54:0.99: #NUM!:0.00:#NUM!), (Vulvovaginitis:0:4831:0:54:0.99:#NUM!:0.00:#NUM!), (Epstein-Barr.virus.associated.lymphoproliferative.disorder:26:4805:0:54:0.99:1.00:0.33:0.49), (Hodgkin's.disease.stage.IV:25:4806:0:54:0.99:1.00:0.32:0.48), (Chloroma:15:4816:0:54:0.99:1.00:0.22:0.3 6), (Intestinal.transplant:15:4816:0:54:0.99:1.00:0.22:0.36), (Rectal.cancer.stage.IV:14:4817:0:54: 0.99:1.00:0.21:0.34), (Bladder.cancer.recurrent:11:4820:0:54:0.99:1.00:0.17:0.29), (Postpartum.h aemorrhage:10:4821:0:54:0.99:1.00:0.16:0.27), (Bile.duct.adenocarcinoma:9:4822:0:54:0.99:1.00: 0.14:0.25), (Laryngeal.stenosis:9:4822:0:54:0.99:1.00:0.14:0.25), (Lung.operation:9:4822:0:54:0. 99:1.00:0.14:0.25), (Neoplasm.of.thymus:9:4822:0:54:0.99:1.00:0.14:0.25), (Extradural.abscess:8: 4823:0:54:0.99:1.00:0.13:0.23), (Eye.oedema:8:4823:0:54:0.99:1.00:0.13:0.23), (Obstruction.gas tric:8:4823:0:54:0.99:1.00:0.13:0.23), (Proteus.test.positive:8:4823:0:54:0.99:1.00:0.13:0.23), (Tr ansplantation.complication:7:4824:0:54:0.99:1.00:0.11:0.21), (Polyneuropathy.chronic:6:4825:0: 54:0.99:1.00:0.10:0.18), (Renal.dysplasia:6:4825:0:54:0.99:1.00:0.10:0.18), (Aortic.calcification: 5:4826:0:54:0.99:1.00:0.08:0.16), (Endocarditis.prophylaxis:5:4826:0:54:0.99:1.00:0.08:0.16), (E nterocolitis.bacterial:5:4826:0:54:0.99:1.00:0.08:0.16), (Mesenteric.vein.thrombosis:5:4826:0:54: 0.99:1.00:0.08:0.16), (Periorbital.cellulitis:5:4826:0:54:0.99:1.00:0.08:0.16), (Peripheral.artery.by pass:5:4826:0:54:0.99:1.00:0.08:0.16), (Procedural.haemorrhage:5:4826:0:54:0.99:1.00:0.08:0.16), (Tri-iodothyronine.decreased:5:4826:0:54:0.99:1.00:0.08:0.16), (Cerebral.congestion:4:4827:0:54:0.9 9:1.00:0.07:0.13), (Impaired.fasting.glucose:4:4827:0:54:0.99:1.00:0.07:0.13), (Lichen.myxoede matosus:4:4827:0:54:0.99:1.00:0.07:0.13), (Parasitic.infection.prophylaxis:4:4827:0:54:0.99:1.00: 0.07:0.13), (Retinal.degeneration:4:4827:0:54:0.99:1.00:0.07:0.13), (Spinal.myelogram:4:4827:0: 54:0.99:1.00:0.07:0.13), (Biliary.dyskinesia:3:4828:0:54:0.99:1.00:0.05:0.10), (Factor.V.Leiden.c arrier:3:4828:0:54:0.99:1.00:0.05:0.10), (Renal.artery.stenosis:3:4828:0:54:0.99:1.00:0.05:0.10), (Antasthmatic.drug.level:2:4829:0:54:0.99:1.00:0.04:0.07), (Fibrin:2:4829:0:54:0.99:1.00:0.04:0.0 7), (Injury.comeal:2:4829:0:54:0.99:1.00:0.04:0.07), (Pancreatic.pseudocyst:2:4829:0:54:0.99:1.0 0:0.04:0.07), (Vocal.cord.thickening:2:4829:0:54:0.99:1.00:0.04:0.07), (Anhedonia:1:4830:0:54:0 0.99:1.00:0.02:0.04), (Biopsy.prostate:1:4830:0:54:0.99:1.00:0.02:0.04), (Cardiopulmonary.bypass: 1:4830:0:54:0.99:1.00:0.02:0.04), (Palmoplantar.keratoderma:1:4830:0:54:0.99:1.00:0.02:0.04), (Post.viral.fatigue.syndrome:1:4830:0:54:0.99:1.00:0.02:0.04), (Vitamin.E.decreased:1:4830:0:54: 0.99:1.00:0.02:0.04), (Extracorporeal.circulation:0:4830:0:55:0.99:#NUM!:0.00:#NUM!), (Hepati c.lesion:18:4812:0:55:0.99:1.00:0.25:0.40), (Desmoplastic.small.round.cell.tumour:15:4815:0:55: 0.99:1.00:0.21:0.35), (Immunochemotherapy:14:4816:0:55:0.99:1.00:0.20:0.34), (Medical.device. complication:11:4819:0:55:0.99:1.00:0.17:0.29), (Anaemia.postoperative:9:4821:0:55:0.99:1.00: 0.14:0.25), (Granuloma.skin:9:4821:0:55:0.99:1.00:0.14:0.25), (Systemic.lupus.erythematosus.ras h:9:4821:0:55:0.99:1.00:0.14:0.25), (Aura:8:4822:0:55:0.99:1.00:0.13:0.23), (Ecchymosis:8:4822: 0:55:0.99:1.00:0.13:0.23), (Genital.herpes.simplex:8:4822:0:55:0.99:1.00:0.13:0.23), (Lung.conso lidation:8:4822:0:55:0.99:1.00:0.13:0.23), (Epiglottitis:7:4823:0:55:0.99:1.00:0.11:0.20), (Hepatit is.acute:7:4823:0:55:0.99:1.00:0.11:0.20), (Inappropriate.affect:6:4824:0:55:0.99:1.00:0.10:0.18), (Lung.squamous.cell.carcinoma.recurrent:6:4824:0:55:0.99:1.00:0.10:0.18), (Abscess.jaw:5:4825: 0:55:0.99:1.00:0.08:0.15), (Cervical.vertebral.fracture:5:4825:0:55:0.99:1.00:0.08:0.15), (Eye.inf ection.toxoplasmal:5:4825:0:55:0.99:1.00:0.08:0.15), (Papillary.renal.cell.carcinoma:5:4825:0:55:0.99:1.00:0.08:0.15), (Respiratory.acidosis:5:4825:0:55:0.99:1.00:0.08:0.15), (Bladder.transitional. cell.carcinoma.stage.IV:4:4826:0:55:0.99:1.00:0.07:0.13), (Spinal.deformity:4:4826:0:55:0.99:1 0.00:0.07:0.13), (Blood.viscosity.decreased:3:4827:0:55:0.99:1.00:0.05:0.10), (Cat.scratch.disease: 3:4827:0:55:0.99:1.00:0.05:0.10), (Conjunctivitis.viral:3:4827:0:55:0.99:1.00:0.05:0.10), (Culture.positive:3:4827:0:55:0.99:1.00:0.05:0.10), (Epidural.injection:3:4827:0:55:0.99:1.00:0.05:0.10), (Muscle.rupture:3:4827:0:55:0.99:1.00:0.05:0.10), (Transcatheter.arterial.chemoembolisation:3:48 27:0:55:0.99:1.00:0.05:0.10), (Valvuloplasty.cardiac:3:4827:0:55:0.99:1.00:0.05:0.10), (Bronchiti s.viral:2:4828:0:55:0.99:1.00:0.04:0.07), (Croup.infectious:2:4828:0:55:0.99:1.00:0.04:0.07), (Piri formis.syndrome:2:4828:0:55:0.99:1.00:0.04:0.07), (Abnormal.sensation.in.eye:1:4829:0:55:0.99: 1.00:0.02:0.04), (Diffuse.panbronchiolitis:1:4829:0:55:0.99:1.00:0.02:0.04), (Endocarditis.candid a:1:4829:0:55:0.99:1.00:0.02:0.04), (Flashback:1:4829:0:55:0.99:1.00:0.02:0.04), (Graft.infection: 1:4829:0:55:0.99:1.00:0.02:0.04), (Drug.rehabilitation:0:4829:0:56:0.99:#NUM!:0.00:#NUM!), (Peritoneal.abscess:0:4829:0:56:0.99:#NUM!:0.00:#NUM!), (Senile.psychosis:0:4829:0:56:0.99:#NUM!:0.00:#NUM!), (Vaginal.prolapse:0:4829:0:56:0.99:#NUM!:0.00:#NUM!), (Ectopic.pregna ncy:14:4815:0:56:0.99:1.00:0.20:0.33), (Osteosarcoma.recurrent:13:4816:0:56:0.99:1.00:0.19:0.3 2), (Rectosigmoid.cancer.stage.IV:12:4817:0:56:0.99:1.00:0.18:0.30), (Gingival.swelling:9:4820: 0:56:0.99:1.00:0.14:0.24), (Gastroenteritis.eosinophilic:8:4821:0:56:0.99:1.00:0.13:0.22), (Protein.urine:7:4822:0:56:0.99:1.00:0.11:0.20), (Testicular.cancer.metastatic:7:4822:0:56:0.99:1.00:0.11: 0.20), (Pickwickian.syndrome:6:4823:0:56:0.99:1.00:0.10:0.18), (Underweight:6:4823:0:56:0.99: 1.00:0.10:0.18), (Arthroscopy:5:4824:0:56:0.99:1.00:0.08:0.15), (Nuclear.magnetic.resonance.ima ging.breast:5:4824:0:56:0.99:1.00:0.08:0.15), (Malignant.nipple.neoplasm:4:4825:0:56:0.99:1.00: 0.07:0.13), (Syringomyelia:4:4825:0:56:0.99:1.00:0.07:0.13), (Amputation:3:4826:0:56:0.99:1.00: 0.05:0.10), (Hepatobiliary.scan:3:4826:0:56:0.99:1.00:0.05:0.10), (Shift.work.disorder:3:4826:0:5 6:0.99:1.00:0.05:0.10), (Atrophy.of.globe:2:4827:0:56:0.99:1.00:0.03:0.07), (Myotonia:2:4827:0: 56:0.99:1.00:0.03:0.07), (Supraventricular.tachyarrhythmia:2:4827:0:56:0.99:1.00:0.03:0.07), (An tidepressant.drug.level:1:4828:0:56:0.99:1.00:0.02:0.03), (Faeces.discoloured:1:4828:0:56:0.99:1 0.00:0.02:0.03), (Gastroenteritis.staphylococcal:1:4828:0:56:0.99:1.00:0.02:0.03), (Homocystinae mia:1:4828:0:56:0.99:1.00:0.02:0.03), (Infected.bite:1:4828:0:56:0.99:1.00:0.02:0.03), (Pneumoc oniosis:1:4828:0:56:0.99:1.00:0.02:0.03), (Shunt.occlusion:1:4828:0:56:0.99:1.00:0.02:0.03), (Va scular.purpura:1:4828:0:56:0.99:1.00:0.02:0.03), (Influenza.A.virus.test.positive:0:4828:0:57:0.9 9:#NUM!:0.00:#NUM!), (Adenosquamous.cell.lung.cancer:15:4813:0:57:0.99:1.00:0.21:0.34), (Malignant.histiocytosis:14:4814:0:57:0.99:1.00:0.20:0.33), (Chylothorax:12:4816:1:56:0.99:0.92: 0.18:0.30), (Calciphylaxis:11:4817:0:57:0.99:1.00:0.16:0.28), (Fear.of.injection:9:4819:0:57:0.99: 1.00:0.14:0.24), (Soft.tissue.neoplasm:9:4819:0:57:0.99:1.00:0.14:0.24), (Inclusion.body.myositis: 8:4820:0:57:0.99:1.00:0.12:0.22), (Parathyroid.tumour.malignant:8:4820:0:57:0.99:1.00:0.12:0.2 2), (Menstruation.delayed:7:4821:0:57:0.99:1.00:0.11:0.20), (Poliomyelitis:6:4822:0:57:0.99:1.00: 0.10:0.17), (Prostatic.operation:6:4822:0:57:0.99:1.00:0.10:0.17), (Bile.duct.obstruction:5:4823:0: 57:0.99:1.00:0.08:0.15), (Dacryostenosis.acquired:5:4823:0:57:0.99:1.00:0.08:0.15), (Eyelid.oed ema:5:4823:0:57:0.99:1.00:0.08:0.15), (Gastrointestinal.candidiasis:5:4823:0:57:0.99:1.00:0.08:0 0.15), (Mesothelioma.malignant:5:4823:0:57:0.99:1.00:0.08:0.15), (Morganella.infection:5:4823:0: 57:0.99:1.00:0.08:0.15), (Small.for.dates.baby:5:4823:0:57:0.99:1.00:0.08:0.15), (Tinea.manuum: 5:4823:0:57:0.99:1.00:0.08:0.15), (Blood.albumin:4:4824:0:57:0.99:1.00:0.07:0.12), (Hepatic.infe ction:4:4824:0:57:0.99:1.00:0.07:0.12), (Carotid.artery.stent.insertion:3:4825:0:57:0.99:1.00:0.05: 0.10), (Empty.sella.syndrome:3:4825:0:57:0.99:1.00:0.05:0.10), (Personality.change:3:4825:0:57: 0.99:1.00:0.05:0.10), (Porphyria:3:4825:0:57:0.99:1.00:0.05:0.10), (Prothrombin.level.increased: 3:4825:0:57:0.99:1.00:0.05:0.10), (Vitamin.C.decreased:3:4825:0:57:0.99:1.00:0.05:0.10), (Crani al.nerve.disorder:2:4826:0:57:0.99:1.00:0.03:0.07), (Dissociative.identity.disorder:2:4826:0:57:0. 99:1.00:0.03:0.07), (Eosinophilic.pustulosis:2:4826:0:57:0.99:1.00:0.03:0.07), (Marasmus:2:4826: 0:57:0.99:1.00:0.03:0.07), (Unevaluable.therapy:2:4826:0:57:0.99:1.00:0.03:0.07), (Akinesia:1:48 27:0:57:0.99:1.00:0.02:0.03), (Dyssomnia:1:4827:0:57:0.99:1.00:0.02:0.03), (Otorrhoea:1:4827:0: 57:0.99:1.00:0.02:0.03), (Ewing's.sarcoma.metastatic:15:4812:0:58:0.99:1.00:0.21:0.34), (Diffuse.large.B-cell.lymphoma.stage.II:14:4813:0:58:0.99:1.00:0.19:0.33), (Glomerulonephritis.rapidly.progressi ve:14:4813:0:58:0.99:1.00:0.19:0.33), (Cerebral.venous.thrombosis:11:4816:0:58:0.99:1.00:0.16: 0.28), (Ocular.pemphigoid:11:4816:0:58:0.99:1.00:0.16:0.28), (Retinal.neovascularisation:11:481 6:0:58:0.99:1.00:0.16:0.28), (Urethritis:9:4818:0:58:0.99:1.00:0.13:0.24), (Adrenal.gland.cancer. metastatic:7:4820:0:58:0.99:1.00:0.11:0.19), (Autoimmune.pancreatitis:7:4820:0:58:0.99:1.00:0. 11:0.19), (Coma:6:4821:0:58:0.99:1.00:0.09:0.17), (Gastrointestinal.injury:6:4821:0:58:0.99:1.00: 0.09:0.17), (Hypervigilance:6:4821:0:58:0.99:1.00:0.09:0.17), (Nerve.degeneration:6:4821:0:58:0 0.99:1.00:0.09:0.17), (Compulsions:5:4822:0:58:0.99:1.00:0.08:0.15), (Plasma.protein.metabolism. disorder:5:4822:0:58:0.99:1.00:0.08:0.15), (Allergy.to.chemicals:4:4823:0:58:0.99:1.00:0.06:0.12), (Bums.second.degree:4:4823:0:58:0.99:1.00:0.06:0.12), (Endarterectomy:4:4823:0:58:0.99:1.00: 0.06:0.12), (Haemophilus.infection:4:4823:0:58:0.99:1.00:0.06:0.12), (Intestinal.ulcer:4:4823:0:5 8:0.99:1.00:0.06:0.12), (Mitochondrial.cytopathy:4:4823:0:58:0.99:1.00:0.06:0.12), (Peripheral.e mbolism:4:4823:0:58:0.99:1.00:0.06:0.12), (Sigmoidoscopy:4:4823:0:58:0.99:1.00:0.06:0.12), (A bulia:3:4824:0:58:0.99:1.00:0.05:0.09), (Deep.vein.thrombosis.postoperative:3:4824:0:58:0.99:1. 00:0.05:0.09), (Hand.fracture:3:4824:0:58:0.99:1.00:0.05:0.09), (Methylenetetrahydrofolate.reduc tase.gene.mutation:3:4824:0:58:0.99:1.00:0.05:0.09), (Paraesthesia.oral:3:4824:0:58:0.99:1.00:0. 05:0.09), (Post.procedural.constipation:3:4824:0:58:0.99:1.00:0.05:0.09), (Total.lung.capacity.dec reased:3:4824:0:58:0.99:1.00:0.05:0.09), (Vestibular.migraine:3:4824:0:58:0.99:1.00:0.05:0.09), (Computerised.tomogram.pelvis:2:4825:0:58:0.99:1.00:0.03:0.06), (Abscess.drainage:1:4826:0:58: 0.99:1.00:0.02:0.03), (Oesophageal.irritation:1:4826:0:58:0.99:1.00:0.02:0.03), (Colon.injury:0:4 826:0:59:0.99:#NUM!:0.00:#NUM!), (Cystitis.escherichia:0:4826:0:59:0.99:#NUM!:0.00:#NUM !), (Neutrophil.count.abnormal:22:4804:0:59:0.99:1.00:0.27:0.43), (Myasthenic.syndrome:10:481 6:0:59:0.99:1.00:0.14:0.25), (Infectious.disease.carrier:8:4818:0:59:0.99:1.00:0.12:0.21), (Chronic.hepatic.failure:7:4819:0:59:0.99:1.00:0.11:0.19), (Mouth.haemorrhage:7:4819:0:59:0.99:1.00:0.1 1:0.19), (Nasal.irrigation:7:4819:0:59:0.99:1.00:0.11:0.19), (Renal.vasculitis:7:4819:0:59:0.99:1.0 0:0.11:0.19), (Conjunctival.haemorrhage:6:4820:0:59:0.99:1.00:0.09:0.17), (Intervertebral.disc.op eration:6:4820:0:59:0.99:1.00:0.09:0.17), (Neurogenic.bowel:6:4820:0:59:0.99:1.00:0.09:0.17), (On.and.off.phenomenon:6:4820:0:59:0.99:1.00:0.09:0.17), (Pyuria:6:4820:0:59:0.99:1.00:0.09:0. 17), (Abasia:5:4821:0:59:0.99:1.00:0.08:0.14), (Arterial.insufficiency:5:4821:0:59:0.99:1.00:0.08: 0.14), (Renal.failure.acute:5:4821:0:59:0.99:1.00:0.08:0.14), (Bile.output.abnormal:4:4822:0:59:0 0.99:1.00:0.06:0.12), (Choking:4:4822:0:59:0.99:1.00:0.06:0.12), (Injection.site.infection:4:4822:0: 59:0.99:1.00:0.06:0.12), (Death.of.relative:3:4823:0:59:0.99:1.00:0.05:0.09), (Ligament.injury:3:4 823:0:59:0.99:1.00:0.05:0.09), (Pilonidal.cyst:3:4823:0:59:0.99:1.00:0.05:0.09), (Tricuspid.valve. disease:3:4823:0:59:0.99:1.00:0.05:0.09), (Low.birth.weight.baby:2:4824:0:59:0.99:1.00:0.03:0.0 6), (Myeloid.metaplasia:2:4824:0:59:0.99:1.00:0.03:0.06), (Radiculitis:2:4824:0:59:0.99:1.00:0.0 3:0.06), (Blood.corticotrophin.abnormal:1:4825:0:59:0.99:1.00:0.02:0.03), (Sexual.activity.increa sed:1:4825:0:59:0.99:1.00:0.02:0.03), (Wound.infection.bacterial:1:4825:0:59:0.99:1.00:0.02:0.0 3), (Hypoprothrombinaemia:0:4825:0:60:0.99:#NUM!:0.00:#NUM!), (Choriocarcinoma:26:4799: 0:60:0.99:1.00:0.30:0.46), (Anaplastic.lymphoma.kinase.gene.mutation:15:4810:0:60:0.99:1.00:0 0.20:0.33), (Mucosal.ulceration:14:4811:0:60:0.99:1.00:0.19:0.32), (Febrile.convulsion:11:4814:1: 59:0.99:0.92:0.16:0.27), (Parapsoriasis:10:4815:0:60:0.99:1.00:0.14:0.25), (Pulmonary.arterial.pre ssure:10:4815:0:60:0.99:1.00:0.14:0.25), (Nasal.cavity.cancer:9:4816:0:60:0.99:1.00:0.13:0.23), (Parasomnia:9:4816:0:60:0.99:1.00:0.13:0.23), (Cardiolipin.antibody.positive:7:4818:0:60:0.99:1. 00:0.10:0.19), (Death:7:4818:0:60:0.99:1.00:0.10:0.19), (Dysplasia:6:4819:0:60:0.99:1.00:0.09:0. 17), (Idiopathic.intracranial.hypertension:6:4819:0:60:0.99:1.00:0.09:0.17), (Portal.hypertensive.g astropathy:6:4819:0:60:0.99:1.00:0.09:0.17), (Procedural.hypertension:5:4820:0:60:0.99:1.00:0.0 8:0.14), (Blood.cortisol.increased:4:4821:0:60:0.99:1.00:0.06:0.12), (Neuropathic.arthropathy:4:4 821:0:60:0.99:1.00:0.06:0.12), (Ovarian.clear.cell.carcinoma:4:4821:0:60:0.99:1.00:0.06:0.12), (S leep.attacks:4:4821:0:60:0.99:1.00:0.06:0.12), (Mucosal.haemorrhage:3:4822:0:60:0.99:1.00:0.05: 0.09), (Respiratory.fume.inhalation.disorder:3:4822:0:60:0.99:1.00:0.05:0.09), (Tibia.fracture:3:4 822:0:60:0.99:1.00:0.05:0.09), (Transurethral.prostatectomy:3:4822:0:60:0.99:1.00:0.05:0.09), (Bl ood.luteinising.hormone.decreased:1:4824:0:60:0.99:1.00:0.02:0.03), (Embolic.cerebral.infarctio n:1:4824:0:60:0.99:1.00:0.02:0.03), (Shock.haemorrhagic:1:4824:0:60:0.99:1.00:0.02:0.03), (Alka losis:0:4824:0:61:0.99:#NUM!:0.00:#NUM!), (Central.nervous.system.neoplasm:23:4801:0:61:0. 99:1.00:0.27:0.43), (Acute.biphenotypic.leukaemia:18:4806:0:61:0.99:1.00:0.23:0.37), (Infiltratio n.anaesthesia:17:4807:3:58:0.99:0.85:0.23:0.36), (Chronic.graft.versus.host.disease.in.skin:16:48 08:0:61:0.99:1.00:0.21:0.34), (Paraganglion.neoplasm:14:4810:0:61:0.99:1.00:0.19:0.31), (Aborti on.spontaneous:13:4811:0:61:0.99:1.00:0.18:0.30), (B-cell.lymphoma.refractory:13:4811:0:61:0.99:1.00:0.18:0.30), (Hepatitis.B.core.antibody.positive: 13:4811:0:61:0.99:1.00:0.18:0.30), (Keloid.scar:9:4815:0:61:0.99:1.00:0.13:0.23), (Clonus:7:4817: 0:61:0.99:1.00:0.10:0.19), (Nystagmus:7:4817:0:61:0.99:1.00:0.10:0.19), (Seizure.like.phenomen a:7:4817:0:61:0.99:1.00:0.10:0.19), (Temperature.intolerance:7:4817:0:61:0.99:1.00:0.10:0.19), (Convulsion.neonatal:6:4818:0:61:0.99:1.00:0.09:0.16), (Malignant.pituitary.tumour:6:4818:0:61: 0.99:1.00:0.09:0.16), (Perineal.pain:6:4818:0:61:0.99:1.00:0.09:0.16), (Pleurodesis:6:4818:0:61:0. 99:1.00:0.09:0.16), (Postmenopausal.haemorrhage:6:4818:0:61:0.99:1.00:0.09:0.16), (Spinal.cord. infection:6:4818:0:61:0.99:1.00:0.09:0.16), (Hepatitis.C.antibody.positive:5:4819:0:61:0.99:1.00: 0.08:0.14), (Malignant.neoplasm.of.spinal.cord:5:4819:0:61:0.99:1.00:0.08:0.14), (Noninfective.g ingivitis:5:4819:0:61:0.99:1.00:0.08:0.14), (Pneumonia.adenoviral:5:4819:0:61:0.99:1.00:0.08:0. 14), (Neuroma:4:4820:0:61:0.99:1.00:0.06:0.12), (Social.problem:4:4820:0:61:0.99:1.00:0.06:0.1 2), (Tearfulness:4:4820:0:61:0.99:1.00:0.06:0.12), (Mean.arterial.pressure:3:4821:0:61:0.99:1.00: 0.05:0.09), (Stent.patency.maintenance:3:4821:0:61:0.99:1.00:0.05:0.09), (Anorexia.nervosa:2:48 22:0:61:0.99:1.00:0.03:0.06), (Bladder.instillation.procedure:2:4822:0:61:0.99:1.00:0.03:0.06), (C artilage.injury:2:4822:0:61:0.99:1.00:0.03:0.06), (Thymus.disorder:2:4822:0:61:0.99:1.00:0.03:0. 06), (Ventricular.hypokinesia:2:4822:0:61:0.99:1.00:0.03:0.06), (Premature.rupture.of.membranes: 1:4823:0:61:0.99:1.00:0.02:0.03), (Rectocele:1:4823:0:61:0.99:1.00:0.02:0.03), (Small.intestinal. bacterial.overgrowth:1:4823:0:61:0.99:1.00:0.02:0.03), (Spinal.cord.infarction:1:4823:0:61:0.99: 1.00:0.02:0.03), (Hypoosmolar.state:0:4823:0:62:0.99:#NUM!:0.00:#NUM!), (Onycholysis:0:482 3:0:62:0.99:#NUM!:0.00:#NUM!), (Soft.tissue.injury:0:4823:0:62:0.99:#NUM!:0.00:#NUM!), (T richophytosis:0:4823:0:62:0.99:#NUM!:0.00:#NUM!), (Obliterative.bronchiolitis:21:4802:0:62:0 0.99:1.00:0.25:0.40), (Escherichia.pyelonephritis:17:4806:0:62:0.99:1.00:0.22:0.35), (Thymic.canc er.metastatic:17:4806:0:62:0.99:1.00:0.22:0.35), (Rheumatic.heart.disease:13:4810:0:62:0.99:1.0 0:0.17:0.30), (Encephalomyelitis:12:4811:0:62:0.99:1.00:0.16:0.28), (Pancreas.islet.cell.transplant: 11:4812:0:62:0.99:1.00:0.15:0.26), (Anticonvulsant.drug.level:7:4816:0:62:0.99:1.00:0.10:0.18), (Meningioma.malignant:7:4816:0:62:0.99:1.00:0.10:0.18), (Chillblains:6:4817:0:62:0.99:1.00:0.0 9:0.16), (Full.blood.count.abnormal:6:4817:0:62:0.99:1.00:0.09:0.16), (Protein.total.decreased:6:4 817:0:62:0.99:1.00:0.09:0.16), (Cervix.disorder:5:4818:0:62:0.99:1.00:0.07:0.14), (Craniotomy:5: 4818:0:62:0.99:1.00:0.07:0.14), (Cyclothymic.disorder:5:4818:0:62:0.99:1.00:0.07:0.14), (Gingiv al.cancer:5:4818:0:62:0.99:1.00:0.07:0.14), (Blood.growth.hormone.decreased:4:4819:0:62:0.99: 1.00:0.06:0.11), (Diffuse.alopecia:4:4819:0:62:0.99:1.00:0.06:0.11), (Hepatitis.A:4:4819:0:62:0.9 9:1.00:0.06:0.11), (Microalbuminuria:4:4819:0:62:0.99:1.00:0.06:0.11), (Transient.acantholytic.d ermatosis:4:4819:0:62:0.99:1.00:0.06:0.11), (Bedridden:3:4820:0:62:0.99:1.00:0.05:0.09), (Ecze ma.nummular:3:4820:0:62:0.99:1.00:0.05:0.09), (Gastrooesophageal.variceal.haemorrhage.proph ylaxis:3:4820:0:62:0.99:1.00:0.05:0.09), (Schizophrenia,.residual.type:3:4820:0:62:0.99:1.00:0.0 5:0.09), (Peripheral.nerve.lesion:2:4821:0:62:0.99:1.00:0.03:0.06), (Prostate.examination:2:4821: 0:62:0.99:1.00:0.03:0.06), (Schizophreniform.disorder:2:4821:0:62:0.99:1.00:0.03:0.06), (Skin.w ound:2:4821:0:62:0.99:1.00:0.03:0.06), (Dedifferentiated.liposarcoma:1:4822:0:62:0.99:1.00:0.0 2:0.03), (Multi-organ.failure:1:4822:0:62:0.99:1.00:0.02:0.03), (Serratia.infection:1:4822:0:62:0.99:1.00:0.02:0.0 3), (Blood.stem.cell.harvest:19:4803:0:63:0.99:1.00:0.23:0.38), (Acute.disseminated.encephalomy elitis:15:4807:0:63:0.99:1.00:0.19:0.32), (Posterior.reversible.encephalopathy.syndrome:12:4810: 0:63:0.99:1.00:0.16:0.28), (Rehabilitation.therapy:12:4810:0:63:0.99:1.00:0.16:0.28), (Neuropsyc hiatric.lupus:10:4812:0:63:0.99:1.00:0.14:0.24), (Sensitivity.of.teeth:10:4812:0:63:0.99:1.00:0.14: 0.24), (Blood.culture.positive:8:4814:0:63:0.99:1.00:0.11:0.20), (Intracranial.venous.sinus.throm bosis:8:4814:0:63:0.99:1.00:0.11:0.20), (Renal.cyst:8:4814:1:62:0.99:0.89:0.11:0.20), (Splenic.m arginal.zone.lymphoma:8:4814:0:63:0.99:1.00:0.11:0.20), (Breast.abscess:7:4815:1:62:0.99:0.88: 0.10:0.18), (Primary.ciliary.dyskinesia:7:4815:0:63:0.99:1.00:0.10:0.18), (Red.blood.cell.count:7: 4815:0:63:0.99:1.00:0.10:0.18), (Adenocarcinoma.of.the.cervix:6:4816:0:63:0.99:1.00:0.09:0.16), (Cardiac.hypertrophy:6:4816:0:63:0.99:1.00:0.09:0.16), (Contrast.media.reaction:5:4817:0:63:0. 99:1.00:0.07:0.14), (Injection:5:4817:0:63:0.99:1.00:0.07:0.14), (Intraocular.pressure.decreased:5: 4817:0:63:0.99:1.00:0.07:0.14), (Catheterisation.venous:4:4818:0:63:0.99:1.00:0.06:0.11), (Cereb ral.artery.stenosis:4:4818:0:63:0.99:1.00:0.06:0.11), (Coordination.abnormal:4:4818:0:63:0.99:1. 00:0.06:0.11), (Kyphosis:4:4818:0:63:0.99:1.00:0.06:0.1 1), (Respiratory.tract.infection.fungal:4:4 818:0:63:0.99:1.00:0.06:0.11), (Coronary.artery.dilatation:3:4819:0:63:0.99:1.00:0.05:0.09), (Ear. pruritus:3:4819:0:63:0.99:1.00:0.05:0.09), (Haematocrit.abnormal:3:4819:0:63:0.99:1.00:0.05:0.0 9), (Hallucinations,.mixed:3:4819:0:63:0.99:1.00:0.05:0.09), (Meningitis.pneumococcal:3:4819:0: 63:0.99:1.00:0.05:0.09), (Schizophrenia,.catatonic.type:3:4819:0:63:0.99:1.00:0.05:0.09), (Sensati on.of.foreign.body:3:4819:0:63:0.99:1.00:0.05:0.09), (Chest.injury:2:4820:0:63:0.99:1.00:0.03:0. 06), (Nasal.septal.operation:2:4820:0:63:0.99:1.00:0.03:0.06), (Haemorrhagic.stroke:1:4821:0:63: 0.99:1.00:0.02:0.03), (Neck.mass:1:4821:0:63:0.99:1.00:0.02:0.03), (Primitive.neuroectodermal.tu mour:23:4798:0:64:0.99:1.00:0.26:0.42), (Non-Hodgkin's.lymphoma.refractory:20:4801:0:64:0.99:1.00:0.24:0.38), (Haemorrhagic.anaemia:10:4 811:0:64:0.99:1.00:0.14:0.24), (Gastritis.haemorrhagic:9:4812:0:64:0.99:1.00:0.12:0.22), (Respira tory.tract.infection.bacterial:8:4813:0:64:0.99:1.00:0.11:0.20), (Chalazion:7:4814:0:64:0.99:1.00: 0.10:0.18), (Face.injury:7:4814:0:64:0.99:1.00:0.10:0.18), (Fibroma:7:4814:0:64:0.99:1.00:0.10:0. 18), (Growth.failure:6:4815:0:64:0.99:1.00:0.09:0.16), (Malignant.fibrous.histiocytoma:6:4815:0: 64:0.99:1.00:0.09:0.16), (Oropharyngitis.fungal:6:4815:0:64:0.99:1.00:0.09:0.16), (Radiation.inju ry:6:4815:0:64:0.99:1.00:0.09:0.16), (Sleep.disorder.due.to.a.general.medical.condition:6:4815:0: 64:0.99:1.00:0.09:0.16), (Chronic.infantile.neurological.cutaneous.and.articular.syndrome:5:4816:0:64:0.99:1.00:0.07:0.14), (Blood.corticotrophin.decreased:3:4818:0:64:0.99:1.00:0.04:0.09), (Inf antile.apnoea:3:4818:0:64:0.99:1.00:0.04:0.09), (Nonspecific.reaction:3:4818:0:64:0.99:1.00:0.04: 0.09), (Peripheral.artery.stenosis:3:4818:0:64:0.99:1.00:0.04:0.09), (Vitrectomy:3:4818:0:64:0.99: 1.00:0.04:0.09), (Grief.reaction:2:4819:0:64:0.99:1.00:0.03:0.06), (Mineral.deficiency:2:4819:0:6 4:0.99:1.00:0.03:0.06), (Sleep.paralysis:1:4820:0:64:0.99:1.00:0.02:0.03), (Hypergammaglobulina emia.benign.monoclonal:14:4806:0:65:0.99:1.00:0.18:0.30), (Amoebiasis:13:4807:0:65:0.99:1.00: 0.17:0.29), (Ependymoma:11:4809:0:65:0.99:1.00:0.14:0.25), (Nephrectomy:10:4810:0:65:0.99: 1.00:0.13:0.24), (Necrosis:9:4811:0:65:0.99:1.00:0.12:0.22), (Angiotensin.converting.enzyme:8:4 812:0:65:0.99:1.00:0.11:0.20), (Cold.type.haemolytic.anaemia:8:4812:0:65:0.99:1.00:0.11:0.20), ( Delayed.engraftment:8:4812:0:65:0.99:1.00:0.11:0.20), (Pupil.dilation.procedure:8:4812:0:65:0.9 9:1.00:0.11:0.20), (Hyperphagia:7:4813:0:65:0.99:1.00:0.10:0.18), (Infective.spondylitis:7:4813:0: 65:0.99:1.00:0.10:0.18), (Throat.cancer:7:4813:0:65:0.99:1.00:0.10:0.18), (Enterococcus.test.posi tive:6:4814:1:64:0.99:0.86:0.09:0.16), (Obsessive.thoughts:6:4814:0:65:0.99:1.00:0.08:0.16), (Ur eterolithiasis:6:4814:0:65:0.99:1.00:0.08:0.16), (Atopy:5:4815:0:65:0.99:1.00:0.07:0.13), (Labour.pain:5:4815:0:65:0.99:1.00:0.07:0.13), (Malignant.neoplasm.of.thorax:5:4815:0:65:0.99:1.00:0.0 7:0.13), (Oral.surgery:5:4815:0:65:0.99:1.00:0.07:0.13), (Streptococcus.test.positive:5:4815:0:65: 0.99:1.00:0.07:0.13), (Gastrointestinal.surgery:4:4816:0:65:0.99:1.00:0.06:0.11), (Mental.disorder.due.to.a.general.medical.condition:4:4816:0:65:0.99:1.00:0.06:0.11), (Arterial.repair:3:4817:0:6 5:0.99:1.00:0.04:0.08), (Pancreaticoduodenectomy:3:4817:0:65:0.99:1.00:0.04:0.08), (Peripheral.r evascularisation:3:4817:0:65:0.99:1.00:0.04:0.08), (Abdominal.adhesions:2:4818:0:65:0.99:1.00: 0.03:0.06), (Eye.infection.bacterial:2:4818:0:65:0.99:1.00:0.03:0.06), (Oesophageal.haemorrhage: 2:4818:0:65:0.99:1.00:0.03:0.06), (Basal.ganglion.degeneration:1:4819:0:65:0.99:1.00:0.02:0.03), (Blood.follicle.stimulating.hormone.decreased:1:4819:0:65:0.99:1.00:0.02:0.03), (Post-traumatic.headache:0:4819:0:66:0.99:#NUM!:0.00:#NUM!), (Osteosarcoma.metastatic:16:4803: 0:66:0.99:1.00:0.20:0.33), (Angiosarcoma.metastatic:14:4805:0:66:0.99:1.00:0.18:0.30), (Pancrea tic.neoplasm:13:4806:0:66:0.99:1.00:0.16:0.28), (Spindle.cell.sarcoma:10:4809:0:66:0.99:1.00:0. 13:0.23), (Bone.densitometry:9:4810:0:66:0.99:1.00:0.12:0.21), (Squamous.cell.carcinoma.of.the. hypopharynx:9:4810:0:66:0.99:1.00:0.12:0.21), (Urethral.stenosis:9:4810:0:66:0.99:1.00:0.12:0.2 1), (Tendemess:8:4811:0:66:0.99:1.00:0.11:0.20), (Intermittent.explosive.disorder:7:4812:0:66:0. 99:1.00:0.10:0.18), (Bum.oesophageal:6:4813:0:66:0.99:1.00:0.08:0.15), (Dialysis.related.compli cation:6:4813:0:66:0.99:1.00:0.08:0.15), (Vertebrobasilar.insufficiency:6:4813:0:66:0.99:1.00:0.0 8:0.15), (Aortic.arteriosclerosis:5:4814:0:66:0.99:1.00:0.07:0.13), (Cold.urticaria:4:4815:0:66:0.9 9:1.00:0.06:0.11), (Pulmonary.function.test:4:4815:0:66:0.99:1.00:0.06:0.11), (Spinal.laminectom y:4:4815:0:66:0.99:1.00:0.06:0.11), (Computerised.tomogram.head:3:4816:0:66:0.99:1.00:0.04:0. 08), (Conduction.disorder:3:4816:0:66:0.99:1.00:0.04:0.08), (Dental.cosmetic.procedure:3:4816:0: 66:0.99:1.00:0.04:0.08), (Enterobacter.pneumonia:3:4816:0:66:0.99:1.00:0.04:0.08), (Hyperbiliru binaemia:3:4816:0:66:0.99:1.00:0.04:0.08), (Osteosynthesis:3:4816:0:66:0.99:1.00:0.04:0.08), (A ntidepressant.drug.clearance:2:4817:0:66:0.99:1.00:0.03:0.06), (Helicobacter.test.positive:2:4817: 0:66:0.99:1.00:0.03:0.06), (Positive.cardiac.inotropic.effect:2:4817:0:66:0.99:1.00:0.03:0.06), (U biquinone.decreased:2:4817:0:66:0.99:1.00:0.03:0.06), (Lysosomal.acid.lipase.deficiency:1:4818: 0:66:0.99:1.00:0.01:0.03), (Volume.blood.increased:1:4818:0:66:0.99:1.00:0.01:0.03), (Juvenile.p soriatic.arthritis:13:4805:0:67:0.99:1.00:0.16:0.28), (Acute.lymphocytic.leukaemia.(in.remission): 12:4806:0:67:0.99:1.00:0.15:0.26), (Hyperaemia:12:4806:0:67:0.99:1.00:0.15:0.26), (Endometria 1.hyperplasia:9:4809:0:67:0.99:1.00:0.12:0.21), (BRCA1.gene.mutation:8:4810:0:67:0.99:1.00:0. 11:0.19), (Fungal.oesophagitis:8:4810:0:67:0.99:1.00:0.11:0.19), (Endocrine.neoplasm.malignant: 7:4811:0:67:0.99:1.00:0.09:0.17), (Non-small.cell.lung.cancer.stage.III:7:4811:0:67:0.99:1.00:0.09:0.17), (Pancreatic.carcinoma.recurrent: 7:4811:0:67:0.99:1.00:0.09:0.17), (Accident:6:4812:0:67:0.99:1.00:0.08:0.15), (Internal.haemorr hage:6:4812:0:67:0.99:1.00:0.08:0.15), (Primary.amyloidosis:6:4812:0:67:0.99:1.00:0.08:0.15), (Occupational.exposure.to.product:5:4813:0:67:0.99:1.00:0.07:0.13), (Sputum.discoloured:5:4813: 0:67:0.99:1.00:0.07:0.13), (Transitional.cell.cancer.of.the.renal.pelvis.and.ureter:5:4813:0:67:0.9 9:1.00:0.07:0.13), (Carotid.endarterectomy:4:4814:0:67:0.99:1.00:0.06:0.1 1), (Mechanical.urticari a:4:4814:0:67:0.99:1.00:0.06:0.11), (Milk.allergy:4:4814:0:67:0.99:1.00:0.06:0.11), (Procedural.a nxiety:4:4814:0:67:0.99:1.00:0.06:0.11), (Tongue.coated:4:4814:0:67:0.99:1.00:0.06:0.11), (Arter ial.stenosis:3:4815:0:67:0.99:1.00:0.04:0.08), (Frontotemporal.dementia:3:4815:0:67:0.99:1.00:0. 04:0.08), (Quality.of.life.decreased:3:4815:0:67:0.99:1.00:0.04:0.08), (Blood.albumin.abnormal:2: 4816:0:67:0.99:1.00:0.03:0.06), (Brain.natriuretic.peptide.increased:2:4816:0:67:0.99:1.00:0.03: 0.06), (Drug.dispensing.error:2:4816:0:67:0.99:1.00:0.03:0.06), (Osteomyelitis.fungal:2:4816:0:6 7:0.99:1.00:0.03:0.06), (Transient.hypogammaglobulinaemia.of.infancy:2:4816:0:67:0.99:1.00:0. 03:0.06), (Testicular.swelling:1:4817:0:67:0.99:1.00:0.01:0.03), (Cord.blood.transplant.therapy:34: 4783:0:68:0.99:1.00:0.33:0.50), (Metastases.to.ovary:27:4790:0:68:0.99:1.00:0.28:0.44), (Cerebe llar.infarction:14:4803:0:68:0.99:1.00:0.17:0.29), (Alveolar.rhabdomyosarcoma:13:4804:0:68:0.9 9:1.00:0.16:0.28), (Meningitis.viral:11:4806:1:67:0.99:0.92:0.14:0.24), (Penile.cancer:11:4806:0: 68:0.99:1.00:0.14:0.24), (Protein-losing.gastroenteropathy:11:4806:0:68:0.99:1.00:0.14:0.24), (Genital.haemorrhage:10:4807:0:68: 0.99:1.00:0.13:0.23), (Lumbar.puncture:10:4807:0:68:0.99:1.00:0.13:0.23), (Photophobia:10:4807: 0:68:0.99:1.00:0.13:0.23), (Renal.tubular.acidosis:10:4807:0:68:0.99:1.00:0.13:0.23), (Pouchitis: 9:4808:0:68:0.99:1.00:0.12:0.21), (Sinusitis.fungal:9:4808:0:68:0.99:1.00:0.12:0.21), (Blood.iron. increased:8:4809:0:68:0.99:1.00:0.11:0.19), (Oligodendroglioma:8:4809:0:68:0.99:1.00:0.11:0.19), (Allergic.transfusion.reaction:7:4810:0:68:0.99:1.00:0.09:0.17), (Bundle.branch.block:7:4810:0: 68:0.99:1.00:0.09:0.17), (Asperger's.disorder:6:4811:0:68:0.99:1.00:0.08:0.15), (Candida.sepsis:6: 4811:0:68:0.99:1.00:0.08:0.15), (Blood.zinc.decreased:5:4812:0:68:0.99:1.00:0.07:0.13), (Cerebel lar.ataxia:5:4812:0:68:0.99:1.00:0.07:0.13), (Psychogenic.seizure:5:4812:0:68:0.99:1.00:0.07:0.1 3), (Sexually.transmitted.disease:5:4812:0:68:0.99:1.00:0.07:0.13), (Acute.tonsillitis:4:4813:0:68: 0.99:1.00:0.06:0.11), (Xerosis:4:4813:0:68:0.99:1.00:0.06:0.11), (Corneal.dystrophy:3:4814:0:68: 0.99:1.00:0.04:0.08), (Dissociative.disorder:3:4814:0:68:0.99:1.00:0.04:0.08), (Eyeld.disorder:3: 4814:0:68:0.99:1.00:0.04:0.08), (Glucose.urine.present:3:4814:0:68:0.99:1.00:0.04:0.08), (Mucos al.dryness:3:4814:0:68:0.99:1.00:0.04:0.08), (Tissue.sealing:3:4814:0:68:0.99:1.00:0.04:0.08), (B reath.odour:2:4815:0:68:0.99:1.00:0.03:0.06), (Infected.cyst:2:4815:0:68:0.99:1.00:0.03:0.06), (A rterial.catheterisation:1:4816:0:68:0.99:1.00:0.01:0.03), (Partial.lipodystrophy:1:4816:0:68:0.99:1 0.00:0.01:0.03), (Stoma.care:1:4816:0:68:0.99:1.00:0.01:0.03), (Blood.cholesterol.normal:0:4816:0: 69:0.99:#NUM!:0.00:#NUM!), (Increased.viscosity.of.upper.respiratory.secretion:0:4816:0:69:0. 99:#NUM!:0.00:#NUM!), (Thyroid.adenoma:0:4816:0:69:0.99:#NUM!:0.00:#NUM!), (Nephritic. syndrome:18:4798:0:69:0.99:1.00:0.21:0.34), (Trisomy.21:18:4798:0:69:0.99:1.00:0.21:0.34), (Su bstance-induced.psychotic.disorder:15:4801:0:69:0.99:1.00:0.18:0.30), (Visceral.leishmaniasis:15:4801:0: 69:0.99:1.00:0.18:0.30), (Hepatic.fibrosis:14:4802:0:69:0.99:1.00:0.17:0.29), (Antiphospholipid. antibodies.positive:13:4803:0:69:0.99:1.00:0.16:0.27), (Hypotonic.urinary.bladder:13:4803:0:69: 0.99:1.00:0.16:0.27), (Tonic.convulsion:12:4804:1:68:0.99:0.92:0.15:0.26), (Autoimmune.arthriti s:11:4805:0:69:0.99:1.00:0.14:0.24), (Opsoclonus.myoclonus:11:4805:0:69:0.99:1.00:0.14:0.24), (Motor.dysfunction:10:4806:0:69:0.99:1.00:0.13:0.22), (Aromatase.inhibition.therapy:9:4807:0:6 9:0.99:1.00:0.12:0.21), (Breast.cancer.in.situ:8:4808:0:69:0.99:1.00:0.10:0.19), (Diabetic.gastropa resis:7:4809:0:69:0.99:1.00:0.09:0.17), (Hirsutism:6:4810:0:69:0.99:1.00:0.08:0.15), (Immunolog y.test.abnormal:6:4810:0:69:0.99:1.00:0.08:0.15), (Bronchopulmonary.disease:5:4811:0:69:0.99: 1.00:0.07:0.13), (Haemoglobin.increased:5:4811:0:69:0.99:1.00:0.07:0.13), (Infective.exacerbatio n.of.bronchiectasis:5:4811:0:69:0.99:1.00:0.07:0.13), (Blood.parathyroid.hormone.abnormal:4:48 12:0:69:0.99:1.00:0.05:0.10), (Precancerous.cells.present:4:4812:0:69:0.99:1.00:0.05:0.10), (Strep tococcal.sepsis:4:4812:0:69:0.99:1.00:0.05:0.10), (Thrombotic.cerebral.infarction:4:4812:0:69:0. 99:1.00:0.05:0.10), (Anal.fungal.infection:3:4813:0:69:0.99:1.00:0.04:0.08), (Pruritus.allergic:3:4 813:0:69:0.99:1.00:0.04:0.08), (Escherichia.test.positive:2:4814:0:69:0.99:1.00:0.03:0.05), (Genit al.pain:2:4814:0:69:0.99:1.00:0.03:0.05), (Primary.hypogonadism:2:4814:0:69:0.99:1.00:0.03:0.0 5), (Eustachian.tube.disorder:1:4815:0:69:0.99:1.00:0.01:0.03), (Acute.febrile.neutrophilic.dermat osis:26:4789:1:69:0.99:0.96:0.27:0.43), (Colon.neoplasm:18:4797:0:70:0.99:1.00:0.20:0.34), (Hy pereosinophilic.syndrome:16:4799:0:70:0.99:1.00:0.19:0.31), (Persistent.foetal.circulation:12:48 03:0:70:0.99:1.00:0.15:0.26), (Systolic.dysfunction:12:4803:0:70:0.99:1.00:0.15:0.26), (Oestroge n.receptor.assay.positive:11:4804:0:70:0.99:1.00:0.14:0.24), (Pneumonia.cryptococcal:11:4804:0: 70:0.99:1.00:0.14:0.24), (Episcleritis:9:4806:0:70:0.99:1.00:0.11:0.20), (Refractory.cytopenia.wit h.multilineage.dysplasia:9:4806:0:70:0.99:1.00:0.11:0.20), (Trigeminal.nerve.disorder:9:4806:0:7 0:0.99:1.00:0.11:0.20), (Drug.effect.decreased:8:4807:0:70:0.99:1.00:0.10:0.19), (Vascular.calcifi cation:8:4807:0:70:0.99:1.00:0.10:0.19), (Galactorrhoea:7:4808:0:70:0.99:1.00:0.09:0.17), (Cutan eous.symptom:6:4809:0:70:0.99:1.00:0.08:0.15), (Elderly:6:4809:0:70:0.99:1.00:0.08:0.15), (Mus cle.building.therapy:6:4809:0:70:0.99:1.00:0.08:0.15), (Respiratory.rate.decreased:6:4809:0:70:0. 99:1.00:0.08:0.15), (Blood.erythropoietin.abnormal:5:4810:0:70:0.99:1.00:0.07:0.13), (Hyperadre nocorticism:5:4810:0:70:0.99:1.00:0.07:0.13), (Bulimia.nervosa:4:4811:0:70:0.99:1.00:0.05:0.10), (Cholecystitis.chronic:4:4811:0:70:0.99:1.00:0.05:0.10), (Disturbance.in.social.behaviour:4:481 1:0:70:0.99:1.00:0.05:0.10), (Positron.emission.tomogram:4:4811:0:70:0.99:1.00:0.05:0.10), (Col onic.lavage:3:4812:0:70:0.99:1.00:0.04:0.08), (Erythromelalgia:3:4812:0:70:0.99:1.00:0.04:0.08), (Biopsy.liver:2:4813:0:70:0.99:1.00:0.03:0.05), (Elevated.mood:2:4813:0:70:0.99:1.00:0.03:0.05), (Saliva.altered:2:4813:0:70:0.99:1.00:0.03:0.05), (Stress.at.work:1:4814:0:70:0.99:1.00:0.01:0.0 3), (Troponin.increased:1:4814:0:70:0.99:1.00:0.01:0.03), (Cardiac.pharmacologic.stress.test:0:48 14:0:71:0.99:#NUM!:0.00:#NUM!), (Low.density.lipoprotein.decreased:0:4814:0:71:0.99:#NUM !:0.00:#NUM!), (Miliaria:0:4814:0:71:0.99:#NUM!:0.00:#NUM!), (Immobile:14:4800:0:71:0.99: 1.00:0.16:0.28), (Desmoid.tumour:11:4803:0:71:0.99:1.00:0.13:0.24), (Colectomy:10:4804:0:71:0 0.99:1.00:0.12:0.22), (Amino.acid.level.decreased:8:4806:0:71:0.99:1.00:0.10:0.18), (Insulinoma:7: 4807:0:71:0.99:1.00:0.09:0.16), (Suprapubic.pain:7:4807:0:71:0.99:1.00:0.09:0.16), (Keratosis.fo llicular:6:4808:0:71:0.99:1.00:0.08:0.14), (Aortic.thrombosis:5:4809:0:71:0.99:1.00:0.07:0.12), (Connective.tissue.neoplasm:5:4809:0:71:0.99:1.00:0.07:0.12), (Epithelioid.mesothelioma:5:4809: 0:71:0.99:1.00:0.07:0.12), (Peripheral.artery.occlusion:5:4809:0:71:0.99:1.00:0.07:0.12), (Red.blo od.cell.count.abnormal:5:4809:0:71:0.99:1.00:0.07:0.12), (Rubber.sensitivity:5:4809:0:71:0.99:1. 00:0.07:0.12), (Adjustment.disorder.with.anxiety:4:4810:0:71:0.99:1.00:0.05:0.10), (Pseudomonal.bacteraemia:4:4810:0:71:0.99:1.00:0.05:0.10), (Cauda.equina.syndrome:3:4811:0:71:0.99:1.00:0 0.04:0.08), (Gluten.sensitivity:3:4811:0:71:0.99:1.00:0.04:0.08), (Respiratory.moniliasis:3:4811:0: 71:0.99:1.00:0.04:0.08), (Vulvitis:3:4811:0:71:0.99:1.00:0.04:0.08), (Hepatomegaly:2:4812:0:71: 0.99:1.00:0.03:0.05), (Pancreatic.cyst:2:4812:0:71:0.99:1.00:0.03:0.05), (Graft.versus.host.disease.in.lung:15:4798:0:72:0.99:1.00:0.17:0.29), (Anal.squamous.cell.carcinoma:13:4800:0:72:0.99:1. 00:0.15:0.27), (Vogt-Koyanagi-Harada.syndrome:11:4802:0:72:0.99:1.00:0.13:0.23), (Dyshidrotic.eczema:10:4803:1:71:0.99:0.9 1:0.12:0.22), (Glomerulonephritis.minimal.lesion:10:4803:0:72:0.99:1.00:0.12:0.22), (Insulin.resi stant.diabetes:9:4804:0:72:0.99:1.00:0.11:0.20), (Breast.feeding:8:4805:0:72:0.99:1.00:0.10:0.18), (Petechiae:8:4805:0:72:0.99:1.00:0.10:0.18), (Anogenital.warts:5:4808:0:72:0.99:1.00:0.06:0.12), (Conjunctival.hyperaemia:5:4808:0:72:0.99:1.00:0.06:0.12), (Cystinuria:5:4808:0:72:0.99:1.00:0 0.06:0.12), (Hemiplegic.migraine:5:4808:0:72:0.99:1.00:0.06:0.12), (Dopaminergic.drug.therapy:4: 4809:0:72:0.99:1.00:0.05:0.10), (Excessive.cerumen.production:3:4810:0:72:0.99:1.00:0.04:0.08), (Drug.resistance:1:4812:0:72:0.99:1.00:0.01:0.03), (Obsessive-compulsive.personality.disorder:0:4812:0:73:0.99:#NUM!:0.00:#NUM!), (T-cell.prolymphocytic.leukaemia:17:4795:0:73:0.99:1.00:0.19:0.32), (Normochromic.normocytic.a naemia:11:4801:0:73:0.99:1.00:0.13:0.23), (Small.intestine.carcinoma.metastatic:11:4801:0:73:0. 99:1.00:0.13:0.23), (Chemotherapy.cytokine.prophylaxis:10:4802:0:73:0.99:1.00:0.12:0.22), (Ly mphopenia:9:4803:0:73:0.99:1.00:0.11:0.20), (Intraocular.melanoma:8:4804:0:73:0.99:1.00:0.10: 0.18), (Retinal.artery.occlusion:8:4804:0:73:0.99:1.00:0.10:0.18), (Large.cell.lung.cancer:7:4805: 0:73:0.99:1.00:0.09:0.16), (Menometrorrhagia:7:4805:0:73:0.99:1.00:0.09:0.16), (Panniculitis:7:4 805:0:73:0.99:1.00:0.09:0.16), (Arnold-Chiari.malformation:6:4806:0:73:0.99:1.00:0.08:0.14), (Carcinoid.tumour.of.the.stomach:6:4806: 0:73:0.99:1.00:0.08:0.14), (Catheter.site.related.reaction:6:4806:0:73:0.99:1.00:0.08:0.14), (Hyper emesis.gravidarum:6:4806:0:73:0.99:1.00:0.08:0.14), (Lupus.endocarditis:6:4806:0:73:0.99:1.00: 0.08:0.14), (Carcinoid.tumour.of.the.gastrointestinal.tract:5:4807:0:73:0.99:1.00:0.06:0.12), (Hae mangiopericytoma:5:4807:0:73:0.99:1.00:0.06:0.12), (Postoperative.thrombosis:5:4807:0:73:0.99: 1.00:0.06:0.12), (Cardiac.aneurysm:4:4808:0:73:0.99:1.00:0.05:0.10), (Gastric.operation:3:4809: 0:73:0.99:1.00:0.04:0.08), (Spinal.cord.neoplasm:3:4809:0:73:0.99:1.00:0.04:0.08), (Tubular.brea st.carcinoma:3:4809:0:73:0.99:1.00:0.04:0.08), (Nephrostomy:2:4810:0:73:0.99:1.00:0.03:0.05), (Neuropsychiatric.symptoms:2:4810:0:73:0.99:1.00:0.03:0.05), (Sinus.arrhythmia:2:4810:0:73:0.9 9:1.00:0.03:0.05), (X-ray:2:4810:0:73:0.99:1.00:0.03:0.05), (Oesophageal.squamous.cell.carcinoma:22:4789:0:74:0.98: 1.00:0.23:0.37), (Henoch-Schonlein.purpura:19:4792:0:74:0.98:1.00:0.20:0.34), (Colorectal.cancer.recurrent:13:4798:0:74: 0.98:1.00:0.15:0.26), (Pulmonary.alveolar.haemorrhage:13:4798:0:74:0.98:1.00:0.15:0.26), (Extu bation:11:4800:1:73:0.98:0.92:0.13:0.23), (Blood.immunoglobulin.G.increased:7:4804:0:74:0.98: 1.00:0.09:0.16), (Device.priming:6:4805:0:74:0.98:1.00:0.08:0.14), (Drug.provocation.test:6:4805: 0:74:0.98:1.00:0.08:0.14), (Incision.site.infection:6:4805:0:74:0.98:1.00:0.08:0.14), (Malignant. mediastinal.neoplasm:6:4805:0:74:0.98:1.00:0.08:0.14), (Hereditary.haemochromatosis:5:4806:0: 74:0.98:1.00:0.06:0.12), (Idiopathic.angioedema:5:4806:0:74:0.98:1.00:0.06:0.12), (Tinea.versico lour:5:4806:0:74:0.98:1.00:0.06:0.12), (Citrobacter.infection:4:4807:0:74:0.98:1.00:0.05:0.10), (E xposure.to.chemical.pollution:4:4807:0:74:0.98:1.00:0.05:0.10), (Gastrointestinal.tube.insertion:4: 4807:0:74:0.98:1.00:0.05:0.10), (Wisdom.teeth.removal:4:4807:0:74:0.98:1.00:0.05:0.10), (Genit al.rash:3:4808:0:74:0.98:1.00:0.04:0.08), (Parainfluenzae.virus.infection:3:4808:0:74:0.98:1.00:0. 04:0.08), (Sleep.disorder.due.to.general.medical.condition,.insomnia.type:3:4808:0:74:0.98:1.00: 0.04:0.08), (Excoriation:2:4809:0:74:0.98:1.00:0.03:0.05), (High.frequency.ablation:2:4809:0:74: 0.98:1.00:0.03:0.05), (Testicular.pain:2:4809:0:74:0.98:1.00:0.03:0.05), (Hodgkin's.disease.nodul ar.sclerosis:29:4781:0:75:0.98:1.00:0.28:0.44), (Congenital.anomaly:16:4794:0:75:0.98:1.00:0.18: 0.30), (Scedosporium.infection:16:4794:0:75:0.98:1.00:0.18:0.30), (Cutaneous.sarcoidosis:15:47 95:1:74:0.98:0.94:0.17:0.29), (Autoimmune.hypothyroidism:12:4798:0:75:0.98:1.00:0.14:0.24), (Streptococcal.bacteraemia:10:4800:0:75:0.98:1.00:0.12:0.21), (Targeted.cancer.therapy:10:4800: 0:75:0.98:1.00:0.12:0.21), (Bowen's.disease:8:4802:0:75:0.98:1.00:0.10:0.18), (Porphyria.acute:7: 4803:0:75:0.98:1.00:0.09:0.16), (Respiratory.tract.infection.viral:7:4803:0:75:0.98:1.00:0.09:0.16), (Salmonellosis:6:4804:0:75:0.98:1.00:0.07:0.14), (Self.injurious.behaviour:6:4804:0:75:0.98:1. 00:0.07:0.14), (Dermatophytosis:5:4805:0:75:0.98:1.00:0.06:0.12), (Haemophilia.A.with.anti.fact or.VIII:5:4805:0:75:0.98:1.00:0.06:0.12), (Rhinalgia:5:4805:0:75:0.98:1.00:0.06:0.12), (Skin.toxi city:5:4805:0:75:0.98:1.00:0.06:0.12), (Toe.amputation:5:4805:0:75:0.98:1.00:0.06:0.12), (Tooth. fracture:5:4805:0:75:0.98:1.00:0.06:0.12), (Hyperhomocysteinaemia:4:4806:0:75:0.98:1.00:0.05: 0.10), (Klinefelter's.syndrome:4:4806:0:75:0.98:1.00:0.05:0.10), (Tendon.injury:4:4806:0:75:0.98: 1.00:0.05:0.10), (Ventricular.assist.device.insertion:4:4806:0:75:0.98:1.00:0.05:0.10), (Eustachia n.tube.dysfunction:3:4807:0:75:0.98:1.00:0.04:0.07), (Renal.abscess:3:4807:0:75:0.98:1.00:0.04: 0.07), (Parosmia:2:4808:0:75:0.98:1.00:0.03:0.05), (Cystathionine.beta-synthase.deficiency:1:4809:0:75:0.98:1.00:0.01:0.03), (Insomnia.related.to.another.mental.conditi on:0:4809:0:76:0.98:#NUM!:0.00:#NUM!), (Nephroblastoma:23:4786:0:76:0.98:1.00:0.23:0.38), (Renal.and.pancreas.transplant:22:4787:0:76:0.98:1.00:0.22:0.37), (Metastases.to.pelvis:21:4788: 0:76:0.98:1.00:0.22:0.36), (Palindromic.rheumatism:19:4790:0:76:0.98:1.00:0.20:0.33), (Develop mental.delay:18:4791:0:76:0.98:1.00:0.19:0.32), (Autoinflammatory.disease:16:4793:0:76:0.98:1 0.00:0.17:0.30), (Right-to-left.cardiac.shunt:16:4793:0:76:0.98:1.00:0.17:0.30), (Hepatorenal.syndrome:11:4798:0:76:0.98: 1.00:0.13:0.22), (Herpes.simplex.encephalitis:11:4798:0:76:0.98:1.00:0.13:0.22), (Vitiligo:10:479 9:0:76:0.98:1.00:0.12:0.21), (Hypoventilation:9:4800:0:76:0.98:1.00:0.11:0.19), (Contraceptive.i mplant:8:4801:0:76:0.98:1.00:0.10:0.17), (Haemorrhage.intracranial:8:4801:0:76:0.98:1.00:0.10: 0.17), (Ischaemic.cerebral.infarction:7:4802:0:76:0.98:1.00:0.08:0.16), (Skin.odour.abnormal:7:48 02:0:76:0.98:1.00:0.08:0.16), (Androgen.therapy:6:4803:0:76:0.98:1.00:0.07:0.14), (Circulating.a nticoagulant:6:4803:0:76:0.98:1.00:0.07:0.14), (Gastrointestinal.stoma.complication:6:4803:0:76: 0.98:1.00:0.07:0.14), (Urethral.disorder:6:4803:0:76:0.98:1.00:0.07:0.14), (Screaming:4:4805:0:7 6:0.98:1.00:0.05:0.10), (Blood.immunoglobulin.E.increased:3:4806:0:76:0.98:1.00:0.04:0.07), (Le g.amputation:3:4806:0:76:0.98:1.00:0.04:0.07), (Amputation.stump.pain:2:4807:0:76:0.98:1.00:0 0.03:0.05), (Cholangitis.acute:2:4807:0:76:0.98:1.00:0.03:0.05), (Substance.dependence:1:4808:0:7 6:0.98:1.00:0.01:0.03), (Allogenic.bone.marrow.transplantation.therapy:25:4783:0:77:0.98:1.00:0 0.25:0.39), (Graft.versus.host.disease.in.liver:25:4783:0:77:0.98:1.00:0.25:0.39), (Metastatic.lymph oma:23:4785:0:77:0.98:1.00:0.23:0.37), (Mantle.cell.lymphoma.refractory:18:4790:0:77:0.98:1.0 0:0.19:0.32), (Laryngeal.squamous.cell.carcinoma:12:4796:0:77:0.98:1.00:0.13:0.24), (Neoplasm. skin:11:4797:0:77:0.98:1.00:0.13:0.22), (Metastatic.uterine.cancer:10:4798:0:77:0.98:1.00:0.11:0 0.21), (Blood.oestrogen.increased:9:4799:0:77:0.98:1.00:0.10:0.19), (Diarrhoea.haemorrhagic:9:47 99:0:77:0.98:1.00:0.10:0.19), (Secondary.hyperthyroidism:8:4800:0:77:0.98:1.00:0.09:0.17), (Coc cydynia:7:4801:0:77:0.98:1.00:0.08:0.15), (Rectal.abscess:7:4801:0:77:0.98:1.00:0.08:0.15), (Mul tiple.drug.therapy:6:4802:0:77:0.98:1.00:0.07:0.13), (Hormone-dependent.prostate.cancer:5:4803:0:77:0.98:1.00:0.06:0.1 1), (Disease.susceptibility:4:4804:0:77: 0.98:1.00:0.05:0.09), (Endometrial.cancer.recurrent:4:4804:0:77:0.98:1.00:0.05:0.09), (Exfoliatio n.glaucoma:4:4804:0:77:0.98:1.00:0.05:0.09), (Gravitational.oedema:4:4804:0:77:0.98:1.00:0.05: 0.09), (High.density.lipoprotein.abnormal:3:4805:0:77:0.98:1.00:0.04:0.07), (Enterocolitis.infecti ous:2:4806:0:77:0.98:1.00:0.03:0.05), (Phosphorus.metabolism.disorder:2:4806:0:77:0.98:1.00:0. 03:0.05), (Hand.dermatitis:1:4807:0:77:0.98:1.00:0.01:0.03), (Angiocentric.lymphoma:30:4777:0: 78:0.98:1.00:0.28:0.43), (Meningitis.tuberculous:24:4783:0:78:0.98:1.00:0.24:0.38), (Small.cell.c arcinoma:20:4787:0:78:0.98:1.00:0.20:0.34), (Ovarian.neoplasm:19:4788:0:78:0.98:1.00:0.20:0.3 3), (Oral.lichen.planus:14:4793:0:78:0.98:1.00:0.15:0.26), (Progressive.relapsing.multiple.sclerosi s:12:4795:0:78:0.98:1.00:0.13:0.24), (Psychotic.symptom:12:4795:0:78:0.98:1.00:0.13:0.24), (Clo stridium.colitis:11:4796:0:78:0.98:1.00:0.12:0.22), (Tuberculous.pleurisy:10:4797:0:78:0.98:1.00: 0.11:0.20), (Adrenalectomy:7:4800:0:78:0.98:1.00:0.08:0.15), (Nail.psoriasis:7:4800:0:78:0.98:1. 00:0.08:0.15), (Sputum.increased:7:4800:0:78:0.98:1.00:0.08:0.15), (Ileostomy:6:4801:0:78:0.98: 1.00:0.07:0.13), (Tricuspid.valve.incompetence:6:4801:0:78:0.98:1.00:0.07:0.13), (Urinary.tract.d iscomfort:5:4802:0:78:0.98:1.00:0.06:0.11), (Prehypertension:4:4803:0:78:0.98:1.00:0.05:0.09), (Sluggishness:3:4804:0:78:0.98:1.00:0.04:0.07), (Low.density.lipoprotein.abnormal:2:4805:0:78:0 0.98:1.00:0.03:0.05), (Sensation.of.blood.flow:2:4805:0:78:0.98:1.00:0.03:0.05), (Tendon.rupture:1: 4806:0:78:0.98:1.00:0.01:0.03), (Metastases.to.adrenals:21:4785:0:79:0.98:1.00:0.21:0.35), (Bloo d.lactate.dehydrogenase.increased:20:4786:0:79:0.98:1.00:0.20:0.34), (Peripheral.nerve.sheath.tu mour.malignant:14:4792:0:79:0.98:1.00:0.15:0.26), (Toxoplasmosis.prophylaxis:13:4793:0:79:0. 98:1.00:0.14:0.25), (Vaginitis.bacterial:10:4796:0:79:0.98:1.00:0.11:0.20), (Abscess.neck:8:4798: 0:79:0.98:1.00:0.09:0.17), (Osteomyelitis.bacterial:8:4798:0:79:0.98:1.00:0.09:0.17), (Perinatal.de pression:8:4798:0:79:0.98:1.00:0.09:0.17), (Disease.risk.factor:7:4799:0:79:0.98:1.00:0.08:0.15), (Vitreous.floaters:7:4799:0:79:0.98:1.00:0.08:0.15), (Hyperkinesia:5:4801:0:79:0.98:1.00:0.06:0. 11), (Volume.blood.decreased:5:4801:0:79:0.98:1.00:0.06:0.11), (Blood.pressure.systolic.abnorm al:4:4802:0:79:0.98:1.00:0.05:0.09), (Herpes.zoster.oticus:4:4802:0:79:0.98:1.00:0.05:0.09), (Blo od.corticotrophin:3:4803:0:79:0.98:1.00:0.04:0.07), (Cystic.fibrosis.pancreatic:3:4803:0:79:0.98: 1.00:0.04:0.07), (Keratosis.pilaris:3:4803:0:79:0.98:1.00:0.04:0.07), (Sickle.cell.anaemia.with.cris is:3:4803:0:79:0.98:1.00:0.04:0.07), (Necrotising.fasciitis:2:4804:0:79:0.98:1.00:0.02:0.05), (Oral.mucosal.blistering:2:4804:0:79:0.98:1.00:0.02:0.05), (Reynold's.syndrome:2:4804:0:79:0.98:1.0 0:0.02:0.05), (Staphylococcal.skin.infection:2:4804:0:79:0.98:1.00:0.02:0.05), (Merycism:1:4805: 0:79:0.98:1.00:0.01:0.02), (Engraftment.syndrome:17:4788:0:80:0.98:1.00:0.18:0.30), (Hydrocep halus:13:4792:1:79:0.98:0.93:0.14:0.25), (Gastroduodenitis:11:4794:0:80:0.98:1.00:0.12:0.22), (O xygen.saturation.abnormal:11:4794:0:80:0.98:1.00:0.12:0.22), (Cholinergic.syndrome:10:4795:0: 80:0.98:1.00:0.11:0.20), (Haematopoietic.neoplasm:10:4795:0:80:0.98:1.00:0.11:0.20), (Ligament.rupture:8:4797:0:80:0.98:1.00:0.09:0.17), (Thoracic.vertebral.fracture:8:4797:0:80:0.98:1.00:0.0 9:0.17), (Cancer.surgery:7:4798:0:80:0.98:1.00:0.08:0.15), (Colostomy:7:4798:0:80:0.98:1.00:0.0 8:0.15), (Exposure.to.toxic.agent:7:4798:0:80:0.98:1.00:0.08:0.15), (Laryngeal.oedema:7:4798:1: 79:0.98:0.88:0.08:0.15), (Papilloma.viral.infection:7:4798:0:80:0.98:1.00:0.08:0.15), (Fractured.s acrum:6:4799:0:80:0.98:1.00:0.07:0.13), (Angiogram.retina:5:4800:0:80:0.98:1.00:0.06:0.11), (Pr ogesterone.decreased:5:4800:0:80:0.98:1.00:0.06:0.1 1), (Pseudomonal.sepsis:5:4800:0:80:0.98:1. 00:0.06:0.11), (Bursitis.infective:4:4801:0:80:0.98:1.00:0.05:0.09), (Ectopic.ACTH.syndrome:4:4 801:0:80:0.98:1.00:0.05:0.09), (Eyelid.ptosis:4:4801:0:80:0.98:1.00:0.05:0.09), (Non-small.cell.lung.cancer.stage.IIIA:4:4801:0:80:0.98:1.00:0.05:0.09), (Dressler's.syndrome:3:4802: 0:80:0.98:1.00:0.04:0.07), (Depression.suicidal:2:4803:0:80:0.98:1.00:0.02:0.05), (Familial.period ic.paralysis:2:4803:0:80:0.98:1.00:0.02:0.05), (Solar.lentigo:2:4803:0:80:0.98:1.00:0.02:0.05), (C holecystitis.infective:1:4804:0:80:0.98:1.00:0.01:0.02), (Endocarditis.enterococcal:0:4804:0:81:0. 98:#NUM!:0.00:#NUM!), (Acute.graft.versus.host.disease.in.skin:26:4778:0:81:0.98:1.00:0.24:0. 39), (Granuloma:23:4781:0:81:0.98:1.00:0.22:0.36), (Chorioretinitis:20:4784:1:80:0.98:0.95:0.20: 0.33), (Haemangioma:16:4788:0:81:0.98:1.00:0.16:0.28), (Eosinophilic.granulomatosis.with.poly angiitis:13:4791:0:81:0.98:1.00:0.14:0.24), (Metastatic.carcinoma.of.the.bladder:11:4793:0:81:0. 98:1.00:0.12:0.21), (Prolactinoma:11:4793:0:81:0.98:1.00:0.12:0.21), (Spleen.disorder:11:4793:0: 81:0.98:1.00:0.12:0.21), (Zygomycosis:11:4793:0:81:0.98:1.00:0.12:0.21), (Non-small.cell.lung.cancer.stage.IIIB:10:4794:0:81:0.98:1.00:0.11:0.20), (Skin.plaque:10:4794:0:81:0 0.98:1.00:0.11:0.20), (Pulmonary.pain:8:4796:0:81:0.98:1.00:0.09:0.16), (Anal.pruritus:7:4797:0:8 1:0.98:1.00:0.08:0.15), (Diplopia:6:4798:0:81:0.98:1.00:0.07:0.13), (Hypometabolism:6:4798:0:8 1:0.98:1.00:0.07:0.13), (Continuous.haemodiafiltration:5:4799:0:81:0.98:1.00:0.06:0.11), (Oesoph ageal.motility.disorder:5:4799:0:81:0.98:1.00:0.06:0.11), (Opportunistic.infection:5:4799:0:81:0. 98:1.00:0.06:0.11), (Tetanus:5:4799:0:81:0.98:1.00:0.06:0.11), (Orthopaedic.procedure:4:4800:0: 81:0.98:1.00:0.05:0.09), (Acute.myeloid.leukaemia.(in.remission):1:4803:0:81:0.98:1.00:0.01:0.0 2), (Assisted.fertilisation:25:4778:0:82:0.98:1.00:0.23:0.38), (Morphoea:15:4788:0:82:0.98:1.00:0 0.15:0.27), (Colon.cancer.stage.III:13:4790:0:82:0.98:1.00:0.14:0.24), (Chorioretinopathy:11:4792: 0:82:0.98:1.00:0.12:0.21), (Pulmonary.vascular.disorder:9:4794:0:82:0.98:1.00:0.10:0.18), (Vascu lar.insufficiency:9:4794:0:82:0.98:1.00:0.10:0.18), (Cystic.fibrosis.hepatic.disease:8:4795:0:82:0. 98:1.00:0.09:0.16), (Vulval.cancer:8:4795:0:82:0.98:1.00:0.09:0.16), (Lumbosacral.radiculopathy: 7:4796:0:82:0.98:1.00:0.08:0.15), (Pseudomonas.test.positive:7:4796:0:82:0.98:1.00:0.08:0.15), (Mite.allergy:6:4797:0:82:0.98:1.00:0.07:0.13), (Lung.infection.pseudomonal:4:4799:0:82:0.98:1. 00:0.05:0.09), (Post-traumatic.neck.syndrome:3:4800:0:82:0.98:1.00:0.04:0.07), (Genitourinary.tract.infection:2:4801: 0:82:0.98:1.00:0.02:0.05), (Ilium.fracture:1:4802:0:82:0.98:1.00:0.01:0.02), (Sinus.pain:1:4802:0:82:0.98:1.00:0.01:0.02), (Heart.transplant.rejection:19:4783:0:83:0.98:1.00:0.19:0.31), (Lymphoc ytosis:13:4789:0:83:0.98:1.00:0.14:0.24), (Protein.S.deficiency:13:4789:0:83:0.98:1.00:0.14:0.24), (Acute.hepatitis.C:11:4791:0:83:0.98:1.00:0.12:0.21), (Protein.urine.present:10:4792:0:83:0.98: 1.00:0.11:0.19), (Sideroblastic.anaemia:9:4793:0:83:0.98:1.00:0.10:0.18), (Pyloric.stenosis:8:479 4:0:83:0.98:1.00:0.09:0.16), (Schizoaffective.disorder.depressive.type:8:4794:0:83:0.98:1.00:0.09: 0.16), (Enzyme.induction:7:4795:0:83:0.98:1.00:0.08:0.14), (Adenoma.benign:6:4796:0:83:0.98: 1.00:0.07:0.13), (Bone.graft:6:4796:0:83:0.98:1.00:0.07:0.13), (Febrile.infection:5:4797:0:83:0.98: 1.00:0.06:0.11), (Macular.hole:5:4797:0:83:0.98:1.00:0.06:0.11), (Vasomotor.rhinitis:5:4797:0:8 3:0.98:1.00:0.06:0.11), (Pleural.infection:3:4799:0:83:0.98:1.00:0.03:0.07), (Reversal.of.opiate.ac tivity:3:4799:0:83:0.98:1.00:0.03:0.07), (Vocal.cord.disorder:3:4799:0:83:0.98:1.00:0.03:0.07), (Bronchial.secretion.retention:2:4800:0:83:0.98:1.00:0.02:0.05), (Metastases.to.kidney:22:4779:0: 84:0.98:1.00:0.21:0.34), (Bone.development.abnormal:13:4788:0:84:0.98:1.00:0.13:0.24), (Sarco ma.uterus:13:4788:0:84:0.98:1.00:0.13:0.24), (Neutrophil.count:12:4789:0:84:0.98:1.00:0.13:0.2 2), (Chondrosarcoma:11:4790:0:84:0.98:1.00:0.12:0.21), (Pelvic.venous.thrombosis:9:4792:0:84: 0.98:1.00:0.10:0.18), (Diaphragmatic.hernia:7:4794:0:84:0.98:1.00:0.08:0.14), (Malignant.neopla sm.of.pleura:7:4794:0:84:0.98:1.00:0.08:0.14), (Nuclear.magnetic.resonance.imaging.abdominal: 7:4794:1:83:0.98:0.88:0.08:0.14), (Aneurysm.repair:6:4795:0:84:0.98:1.00:0.07:0.13), (Limb.oper ation:6:4795:0:84:0.98:1.00:0.07:0.13), (Neutropenic.colitis:6:4795:0:84:0.98:1.00:0.07:0.13), (O ophorectomy:6:4795:0:84:0.98:1.00:0.07:0.13), (Rhinovirus.infection:6:4795:0:84:0.98:1.00:0.07: 0.13), (Platelet.transfusion:5:4796:0:84:0.98:1.00:0.06:0.11), (Axonal.neuropathy:4:4797:0:84:0. 98:1.00:0.05:0.09), (Pancreatic.enzymes.decreased:4:4797:0:84:0.98:1.00:0.05:0.09), (Medical.de vice.site.joint.infection:2:4799:0:84:0.98:1.00:0.02:0.05), (Perichondritis:2:4799:0:84:0.98:1.00:0 0.02:0.05), (Sudden.hearing.loss:2:4799:0:84:0.98:1.00:0.02:0.05), (Bladder.irrigation:1:4800:0:84: 0.98:1.00:0.01:0.02), (Trichorrhexis:1:4800:0:84:0.98:1.00:0.01:0.02), (Varicella.zoster.virus.infe ction:22:4778:0:85:0.98:1.00:0.21:0.34), (Neurosarcoidosis:15:4785:0:85:0.98:1.00:0.15:0.26), (R ectal.cancer.recurrent:13:4787:0:85:0.98:1.00:0.13:0.23), (Rickets:12:4788:0:85:0.98:1.00:0.12:0. 22), (Wolff-Parkinson-White.syndrome:12:4788:0:85:0.98:1.00:0.12:0.22), (Abdominal.symptom:11:4789:0:85:0.98:1.0 0:0.11:0.21), (Vitamin.C:10:4790:0:85:0.98:1.00:0.11:0.19), (Biliary.tract.disorder:9:4791:0:85:0. 98:1.00:0.10:0.17), (Lower.respiratory.tract.infection.fungal:9:4791:0:85:0.98:1.00:0.10:0.17), (A phthous.stomatitis:8:4792:0:85:0.98:1.00:0.09:0.16), (Teething:7:4793:0:85:0.98:1.00:0.08:0.14), (Escherichia.sepsis:6:4794:0:85:0.98:1.00:0.07:0.12), (Muscle.tone.disorder:6:4794:0:85:0.98:1.0 0:0.07:0.12), (Suicidal.behaviour:6:4794:0:85:0.98:1.00:0.07:0.12), (Pneumothorax:5:4795:0:85:0 0.98:1.00:0.06:0.11), (Serum.serotonin.decreased:5:4795:0:85:0.98:1.00:0.06:0.11), (Lung.adenoca rcinoma.recurrent:4:4796:0:85:0.98:1.00:0.04:0.09), (Meralgia.paraesthetica:4:4796:0:85:0.98:1. 00:0.04:0.09), (Appendicectomy:3:4797:0:85:0.98:1.00:0.03:0.07), (Non-Hodgkin's.lymphoma.stage.IV:26:4773:0:86:0.98:1.00:0.23:0.38), (Hypersensitivity.vasculitis:18: 4781:0:86:0.98:1.00:0.17:0.30), (Complications.of.transplanted.lung:12:4787:0:86:0.98:1.00:0.1 2:0.22), (Metastases.to.the.mediastinum:10:4789:0:86:0.98:1.00:0.10:0.19), (Uterine.disorder:10:4 789:0:86:0.98:1.00:0.10:0.19), (Glycogen.storage.disorder:9:4790:0:86:0.98:1.00:0.09:0.17), (Squ amous.cell.carcinoma.of.the.oral.cavity:8:4791:0:86:0.98:1.00:0.09:0.16), (Gastrointestinal.strom al.cancer:7:4792:0:86:0.98:1.00:0.08:0.14), (Prostate.cancer.recurrent:7:4792:0:86:0.98:1.00:0.08: 0.14), (Sodium.retention:7:4792:0:86:0.98:1.00:0.08:0.14), (Scratch:6:4793:0:86:0.98:1.00:0.07:0 0.12), (Brain.stem.infarction:5:4794:0:86:0.98:1.00:0.05:0.10), (Candiduria:5:4794:0:86:0.98:1.00: 0.05:0.10), (Eustachian.tube.obstruction:5:4794:0:86:0.98:1.00:0.05:0.10), (Nasal.septum.deviatio n:4:4795:0:86:0.98:1.00:0.04:0.09), (Dust.allergy:3:4796:0:86:0.98:1.00:0.03:0.07), (Transgender. hormonal.therapy:3:4796:0:86:0.98:1.00:0.03:0.07), (Extraskeletal.myxoid.chondrosarcoma:2:47 97:0:86:0.98:1.00:0.02:0.04), (Lacrimation.disorder:1:4798:0:86:0.98:1.00:0.01:0.02), (Immune.r econstitution.inflammatory.syndrome:46:4752:0:87:0.98:1.00:0.35:0.51), (Felty's.syndrome:19:4 779:0:87:0.98:1.00:0.18:0.30), (SAPHO.syndrome:16:4782:0:87:0.98:1.00:0.16:0.27), (Light.chai n.disease:15:4783:0:87:0.98:1.00:0.15:0.26), (Jaundice:14:4784:0:87:0.98:1.00:0.14:0.24), (Type. 3.diabetes.mellitus:14:4784:1:86:0.98:0.93:0.14:0.24), (Bone.tuberculosis:13:4785:0:87:0.98:1.0 0:0.13:0.23), (Laboratory.test.abnormal:12:4786:0:87:0.98:1.00:0.12:0.22), (Vestibular.disorder:1 2:4786:0:87:0.98:1.00:0.12:0.22), (Nephropathy.toxic:9:4789:0:87:0.98:1.00:0.09:0.17), (Neurofi bromatosis:9:4789:0:87:0.98:1.00:0.09:0.17), (Post.procedural.inflammation:9:4789:0:87:0.98:1. 00:0.09:0.17), (Blood.beta-D-glucan.increased:8:4790:0:87:0.98:1.00:0.08:0.16), (Pelvic.inflammatory.disease:6:4792:0:87:0.9 8:1.00:0.06:0.12), (Dysphoria:5:4793:0:87:0.98:1.00:0.05:0.10), (Disability:3:4795:0:87:0.98:1.00: 0.03:0.06), (Ingrowing.nail:3:4795:0:87:0.98:1.00:0.03:0.06), (Mononeuropathy:3:4795:0:87:0.9 8:1.00:0.03:0.06), (Blood.urea.increased:2:4796:0:87:0.98:1.00:0.02:0.04), (Endoscopic.retrograd e.cholangiopancreatography:2:4796:0:87:0.98:1.00:0.02:0.04), (Septic.arthritis.staphylococcal:2: 4796:0:87:0.98:1.00:0.02:0.04), (Toxic.epidermal.necrolysis:17:4780:0:88:0.98:1.00:0.16:0.28), (I nfectious.colitis:15:4782:0:88:0.98:1.00:0.15:0.25), (Pulmonary.veno-occlusive.disease:15:4782:0:88:0.98:1.00:0.15:0.25), (Laryngospasm:13:4784:0:88:0.98:1.00:0.1 3:0.23), (Drug.level:12:4785:0:88:0.98:1.00:0.12:0.21), (Malignant.neoplasm.of.ampulla.of.Vater: 12:4785:0:88:0.98:1.00:0.12:0.21), (Pneumonia.pneumococcal:11:4786:0:88:0.98:1.00:0.11:0.20), (Decreased.immune.responsiveness:8:4789:0:88:0.98:1.00:0.08:0.15), (Metastases.to.prostate:8: 4789:0:88:0.98:1.00:0.08:0.15), (Sterilisation:8:4789:0:88:0.98:1.00:0.08:0.15), (Axillary.pain:7:4 790:0:88:0.98:1.00:0.07:0.14), (Infected.dermal.cyst:7:4790:0:88:0.98:1.00:0.07:0.14), (Accelerat ed.hypertension:5:4792:0:88:0.98:1.00:0.05:0.10), (Lacrimation.decreased:5:4792:0:88:0.98:1.00: 0.05:0.10), (Mycoplasma.infection:5:4792:0:88:0.98:1.00:0.05:0.10), (Testicular.disorder:5:4792: 0:88:0.98:1.00:0.05:0.10), (Blood.bicarbonate.decreased:4:4793:0:88:0.98:1.00:0.04:0.08), (Weig ht.abnormal:4:4793:0:88:0.98:1.00:0.04:0.08), (Bacterial.test.positive:2:4795:0:88:0.98:1.00:0.02: 0.04), (Product.used.for.unknown.indication:4755:41:84:5:0.98:0.98:1.00:0.99), (Granulocytopen ia:24:4772:0:89:0.98:1.00:0.21:0.35), (B-cell.lymphoma.stage.III:19:4777:0:89:0.98:1.00:0.18:0.30), (Lung.transplant.rejection:19:4777:0: 89:0.98:1.00:0.18:0.30), (Renal.vein.thrombosis:17:4779:0:89:0.98:1.00:0.16:0.28), (Colon.cancer.recurrent:15:4781:0:89:0.98:1.00:0.14:0.25), (Anaemia.folate.deficiency:14:4782:0:89:0.98:1.00: 0.14:0.24), (Pityriasis.rubra.pilaris:12:4784:0:89:0.98:1.00:0.12:0.21), (Allergic.respiratory.symp tom:11:4785:0:89:0.98:1.00:0.11:0.20), (Lip.pain:11:4785:0:89:0.98:1.00:0.11:0.20), (Pancreatect omy:10:4786:0:89:0.98:1.00:0.10:0.18), (Abdominal.neoplasm:9:4787:0:89:0.98:1.00:0.09:0.17), (Comeal.oedema:8:4788:0:89:0.98:1.00:0.08:0.15), (Synovial.sarcoma.metastatic:8:4788:0:89:0. 98:1.00:0.08:0.15), (Gastric.hypomotility:7:4789:0:89:0.98:1.00:0.07:0.14), (Borderline.glaucoma: 6:4790:0:89:0.98:1.00:0.06:0.12), (Hypermagnesaemia:5:4791:0:89:0.98:1.00:0.05:0.10), (Dental.discomfort:4:4792:0:89:0.98:1.00:0.04:0.08), (Lobar.pneumonia:4:4792:0:89:0.98:1.00:0.04:0.0 8), (Loss.of.libido:4:4792:0:89:0.98:1.00:0.04:0.08), (Abdominal.tendemess:3:4793:0:89:0.98:1.0 0:0.03:0.06), (Burnout.syndrome:3:4793:0:89:0.98:1.00:0.03:0.06), (Prothrombin.time.prolonged: 3:4793:0:89:0.98:1.00:0.03:0.06), (Intercostal.neuralgia:2:4794:0:89:0.98:1.00:0.02:0.04), (Treat ment.failure:2:4794:0:89:0.98:1.00:0.02:0.04), (Oppositional.defiant.disorder:20:4775:0:90:0.98: 1.00:0.18:0.31), (Human.herpesvirus.6.infection:18:4777:0:90:0.98:1.00:0.17:0.29), (Undifferenti ated.sarcoma:15:4780:0:90:0.98:1.00:0.14:0.25), (Clear.cell.sarcoma.of.soft.tissue:14:4781:0:90: 0.98:1.00:0.13:0.24), (Sensory.loss:13:4782:0:90:0.98:1.00:0.13:0.22), (Mental.disability:10:4785: 0:90:0.98:1.00:0.10:0.18), (Eye.injury:8:4787:0:90:0.98:1.00:0.08:0.15), (Diabetic.vascular.disor der:7:4788:0:90:0.98:1.00:0.07:0.13), (Fibrocystic.breast.disease:7:4788:0:90:0.98:1.00:0.07:0.13), (Angular.cheilitis:5:4790:0:90:0.98:1.00:0.05:0.10), (Multiple.fractures:5:4790:0:90:0.98:1.00:0 0.05:0.10), (Carbuncle:4:4791:0:90:0.98:1.00:0.04:0.08), (Gastrointestinal.angiodysplasia:2:4793:0: 90:0.98:1.00:0.02:0.04), (Renal.aplasia:2:4793:0:90:0.98:1.00:0.02:0.04), (Thymoma:25:4769:0: 91:0.98:1.00:0.22:0.35), (Acute.monocytic.leukaemia:22:4772:1:90:0.98:0.96:0.20:0.33), (Kaposi's.sarcoma.AIDS.related:20:4774:0:91:0.98:1.00:0.18:0.31), (Blood.growth.hormone.abnormal:19: 4775:0:91:0.98:1.00:0.17:0.29), (Prenatal.care:15:4779:0:91:0.98:1.00:0.14:0.25), (Clonic.convul sion:14:4780:0:91:0.98:1.00:0.13:0.24), (Large.intestine.polyp:14:4780:0:91:0.98:1.00:0.13:0.24), (Hypercorticoidism:13:4781:0:91:0.98:1.00:0.13:0.22), (Pneumonia.viral:13:4781:0:91:0.98:1.00: 0.13:0.22), (Pulmonary.artery.thrombosis:10:4784:0:91:0.98:1.00:0.10:0.18), (Retinal.haemorrha ge:10:4784:0:91:0.98:1.00:0.10:0.18), (Vascular.operation:10:4784:0:91:0.98:1.00:0.10:0.18), (A ptyalism:9:4785:0:91:0.98:1.00:0.09:0.17), (Throat.tightness:7:4787:0:91:0.98:1.00:0.07:0.13), (C onjunctivitis.bacterial:6:4788:0:91:0.98:1.00:0.06:0.12), (Electrolyte.depletion:5:4789:0:91:0.98: 1.00:0.05:0.10), (Blood.pressure.systolic.decreased:4:4790:0:91:0.98:1.00:0.04:0.08), (Chronic.ly mphocytic.leukaemia.(in.remission):4:4790:0:91:0.98:1.00:0.04:0.08), (Scab:4:4790:0:91:0.98:1. 00:0.04:0.08), (Bundle.branch.block.right:2:4792:0:91:0.98:1.00:0.02:0.04), (Oesophageal.rupture: 0:4793:0:92:0.98:#NUM!:0.00:#NUM!), (Hepatoblastoma:26:4767:0:92:0.98:1.00:0.22:0.36), (In fertility.female:21:4772:2:90:0.98:0.91:0.19:0.31), (Multiple.sclerosis.relapse.prophylaxis:15:477 8:0:92:0.98:1.00:0.14:0.25), (Atrioventricular.block.complete:14:4779:0:92:0.98:1.00:0.13:0.23), (Visual.field.defect:12:4781:0:92:0.98:1.00:0.12:0.21), (Mitral.valve.stenosis:11:4782:0:92:0.98: 1.00:0.11:0.19), (Lymphangiosis.carcinomatosa:9:4784:0:92:0.98:1.00:0.09:0.16), (Anaemia.macr ocytic:8:4785:0:92:0.98:1.00:0.08:0.15), (Acral.overgrowth:7:4786:0:92:0.98:1.00:0.07:0.13), (Ne urotoxicity:7:4786:0:92:0.98:1.00:0.07:0.13), (Coronary.artery.thrombosis:6:4787:0:92:0.98:1.00: 0.06:0.12), (Dermatitis.herpetiformis:6:4787:0:92:0.98:1.00:0.06:0.12), (Intestinal.ischaemia:6:4 787:0:92:0.98:1.00:0.06:0.12), (Glycosylated.haemoglobin.decreased:5:4788:0:92:0.98:1.00:0.05: 0.10), (Chloasma:4:4789:0:92:0.98:1.00:0.04:0.08), (Muscle.injury:2:4791:0:92:0.98:1.00:0.02:0. 04), (Oesophageal.achalasia:2:4791:0:92:0.98:1.00:0.02:0.04), (Ovarian.disorder:18:4774:0:93:0. 98:1.00:0.16:0.28), (Seizure.cluster:17:4775:0:93:0.98:1.00:0.15:0.27), (B-cell.lymphoma.recurrent:16:4776:1:92:0.98:0.94:0.15:0.26), (Prophylaxis.of.abortion:16:4776:0: 93:0.98:1.00:0.15:0.26), (Reiter's.syndrome:15:4777:0:93:0.98:1.00:0.14:0.24), (Computerised.to mogram.thorax:10:4782:0:93:0.98:1.00:0.10:0.18), (Granuloma.annulare:10:4782:0:93:0.98:1.00: 0.10:0.18), (Pulmonary.function.test.decreased:9:4783:0:93:0.98:1.00:0.09:0.16), (Tracheobronchi tis:9:4783:0:93:0.98:1.00:0.09:0.16), (Chromosomal.deletion:8:4784:0:93:0.98:1.00:0.08:0.15), (Heparin.neutralisation.therapy:8:4784:0:93:0.98:1.00:0.08:0.15), (Skeletal.injury:8:4784:0:93:0.9 8:1.00:0.08:0.15), (Erdheim-Chester.disease:4:4788:0:93:0.98:1.00:0.04:0.08), (Intemational.normalised.ratio.decreased:4:478 8:0:93:0.98:1.00:0.04:0.08), (Babesiosis:3:4789:0:93:0.98:1.00:0.03:0.06), (Post.concussion.syndr ome:2:4790:0:93:0.98:1.00:0.02:0.04), (Adjustment.disorder.with.mixed.anxiety.and.depressed.m ood:1:4791:0:93:0.98:1.00:0.01:0.02), (Eye.irrigation:21:4770:3:91:0.98:0.88:0.19:0.31), (Latent. autoimmune.diabetes.in.adults:21:4770:0:94:0.98:1.00:0.18:0.31), (Lung.squamous.cell.carcinom a.stage.IV:17:4774:0:94:0.98:1.00:0.15:0.27), (Myelitis:16:4775:0:94:0.98:1.00:0.15:0.25), (Oste opetrosis:16:4775:0:94:0.98:1.00:0.15:0.25), (Ventricular.septal.defect:14:4777:0:94:0.98:1.00:0. 13:0.23), (Toxic.skin.eruption:12:4779:0:94:0.98:1.00:0.11:0.20), (Tumer's.syndrome:8:4783:0:9 4:0.98:1.00:0.08:0.15), (Fungal.test.positive:7:4784:0:94:0.98:1.00:0.07:0.13), (Respiratory.depre ssion:6:4785:0:94:0.98:1.00:0.06:0.11), (Aspiration.bone.marrow:2:4789:0:94:0.98:1.00:0.02:0.0 4), (Gamma-glutamyltransferase.increased:2:4789:0:94:0.98:1.00:0.02:0.04), (Polyneuropathy.idiopathic.prog ressive:0:4790:0:95:0.98:#NUM!:0.00:#NUM!), (Evans.syndrome:24:4766:0:95:0.98:1.00:0.20:0 0.34), (Antisynthetase.syndrome:21:4769:0:95:0.98:1.00:0.18:0.31), (Chemical.contraception:21:4 769:0:95:0.98:1.00:0.18:0.31), (Electroencephalogram.abnormal:20:4770:0:95:0.98:1.00:0.17:0.3 0), (Idiopathic.generalised.epilepsy:17:4773:0:95:0.98:1.00:0.15:0.26), (Atypical.haemolytic.urae mic.syndrome:15:4775:0:95:0.98:1.00:0.14:0.24), (Pyoderma:15:4775:0:95:0.98:1.00:0.14:0.24), (Delayed.graft.function:11:4779:0:95:0.98:1.00:0.10:0.19), (Hypertensive.nephropathy:10:4780:0: 95:0.98:1.00:0.10:0.17), (Leiomyoma:10:4780:0:95:0.98:1.00:0.10:0.17), (Xerophthalmia:10:478 0:0:95:0.98:1.00:0.10:0.17), (Contrast.media.allergy:9:4781:0:95:0.98:1.00:0.09:0.16), (Haemorrh agic.disorder:9:4781:0:95:0.98:1.00:0.09:0.16), (Mononeuritis:9:4781:0:95:0.98:1.00:0.09:0.16), (Hypophysitis:8:4782:0:95:0.98:1.00:0.08:0.14), (Idiopathic.interstitial.pneumonia:8:4782:0:95:0. 98:1.00:0.08:0.14), (Swollen.tongue:8:4782:0:95:0.98:1.00:0.08:0.14), (Macrocytosis:6:4784:0:95: 0.98:1.00:0.06:0.11), (Renal.surgery:5:4785:0:95:0.98:1.00:0.05:0.10), (Systemic.sclerosis.pulmo nary:5:4785:0:95:0.98:1.00:0.05:0.10), (Cervical.myelopathy:4:4786:0:95:0.98:1.00:0.04:0.08), (F ollicle.centre.lymphoma,.follicular.grade.I,.II,.III:20:4769:0:96:0.98:1.00:0.17:0.29), (Aphasia:15: 4774:0:96:0.98:1.00:0.14:0.24), (Hepatitis.B.virus.test:13:4776:0:96:0.98:1.00:0.12:0.21), (Centr al.nervous.system.infection:12:4777:0:96:0.98:1.00:0.11:0.20), (Cardio-respiratory.arrest:11:4778:1:95:0.98:0.92:0.10:0.19), (Pulmonary.haemorrhage:11:4778:0:96:0.98: 1.00:0.10:0.19), (Nasal.obstruction:10:4779:0:96:0.98:1.00:0.09:0.17), (Otitis.media.acute:10:47 79:0:96:0.98:1.00:0.09:0.17), (Clavicle.fracture:7:4782:0:96:0.98:1.00:0.07:0.13), (Peripheral.circ ulatory.failure:7:4782:0:96:0.98:1.00:0.07:0.13), (Malignant.neoplasm.of.islets.of.Langerhans:6:4 783:0:96:0.98:1.00:0.06:0.11), (Mastectomy:6:4783:0:96:0.98:1.00:0.06:0.11), (Rhinitis.prophyla xis:5:4784:0:96:0.98:1.00:0.05:0.09), (Tachypnoea:5:4784:0:96:0.98:1.00:0.05:0.09), (Urinary.tra ct.pain:5:4784:0:96:0.98:1.00:0.05:0.09), (Progressive.supranuclear.palsy:2:4787:0:96:0.98:1.00: 0.02:0.04), (Neoadjuvant.therapy:20:4768:0:97:0.98:1.00:0.17:0.29), (Blast.crisis.in.myelogenous. leukaemia:19:4769:0:97:0.98:1.00:0.16:0.28), (Tuberculin.test.positive:18:4770:0:97:0.98:1.00:0. 16:0.27), (Tracheitis:14:4774:0:97:0.98:1.00:0.13:0.22), (Exposure.to.communicable.disease:12:4 776:0:97:0.98:1.00:0.11:0.20), (Mucositis.management:10:4778:0:97:0.98:1.00:0.09:0.17), (Skin. burning.sensation:9:4779:0:97:0.98:1.00:0.08:0.16), (Stasis.dermatitis:9:4779:0:97:0.98:1.00:0.0 8:0.16), (Chronic.respiratory.disease:8:4780:0:97:0.98:1.00:0.08:0.14), (Haemophilic.arthropathy: 8:4780:0:97:0.98:1.00:0.08:0.14), (Malignant.melanoma.stage.III:8:4780:0:97:0.98:1.00:0.08:0.1 4), (Radiation.oesophagitis:6:4782:0:97:0.98:1.00:0.06:0.11), (Diverticulum.intestinal:5:4783:0:9 7:0.98:1.00:0.05:0.09), (Gastrointestinal.bacterial.infection:5:4783:0:97:0.98:1.00:0.05:0.09), (Ga strointestinal.hypermotility:5:4783:0:97:0.98:1.00:0.05:0.09), (Lacrimal.disorder:4:4784:0:97:0.9 8:1.00:0.04:0.08), (Rapid.eye.movement.sleep.behaviour.disorder:3:4785:0:97:0.98:1.00:0.03:0.0 6), (Urinary.hesitation:3:4785:0:97:0.98:1.00:0.03:0.06), (Foetal.exposure.timing.unspecified:26:4 761:0:98:0.98:1.00:0.21:0.35), (Chlamydial.infection:23:4764:0:98:0.98:1.00:0.19:0.32), (Acute.l ymphocytic.leukaemia.refractory:22:4765:0:98:0.98:1.00:0.18:0.31), (Mantle.cell.lymphoma.stag e.IV:22:4765:0:98:0.98:1.00:0.18:0.31), (Artificial.menopause:16:4771:0:98:0.98:1.00:0.14:0.25), (Vitamin.A.deficiency:12:4775:0:98:0.98:1.00:0.11:0.20), (Vascular.occlusion:9:4778:0:98:0.98: 1.00:0.08:0.16), (Blood.testosterone.increased:8:4779:0:98:0.98:1.00:0.08:0.14), (Brain.operation: 7:4780:0:98:0.98:1.00:0.07:0.13), (Cardiac.ventricular.thrombosis:7:4780:0:98:0.98:1.00:0.07:0.1 3), (Dysarthria:6:4781:0:98:0.98:1.00:0.06:0.11), (Oropharyngeal.discomfort:5:4782:0:98:0.98:1. 00:0.05:0.09), (Vascular.pain:5:4782:0:98:0.98:1.00:0.05:0.09), (Chronic.kidney.disease-mineral.and.bone.disorder:4:4783:0:98:0.98:1.00:0.04:0.08), (Body.fat.disorder:2:4785:0:98:0.98:1.00:0.02:0.04), (Prophylaxis.against.alcoholic.withdrawal.syndrome:2:4785:0:98:0.98:1.00:0.02:0.04), (Hepatitis.viral:32:4754:0:99:0.98:1.00:0.24:0.39), (Bile.duct.stone:17:4769:0:99:0.98:1.00: 0.15:0.26), (Vascular.malformation:17:4769:0:99:0.98:1.00:0.15:0.26), (Lymphoplasmacytoid.ly mphoma/immunocytoma:14:4772:0:99:0.98:1.00:0.12:0.22), (Hypochromic.anaemia:12:4774:0:9 9:0.98:1.00:0.11:0.20), (Soft.tissue.disorder:12:4774:0:99:0.98:1.00:0.11:0.20), (Glossodynia:10:4 776:0:99:0.98:1.00:0.09:0.17), (Pneumonia.legionella:9:4777:0:99:0.98:1.00:0.08:0.15), (Chronic. myeloid.leukaemia.(in.remission):8:4778:0:99:0.98:1.00:0.07:0.14), (Sinus.rhythm:8:4778:0:99:0 0.98:1.00:0.07:0.14), (Urinary.tract.infection.fungal:8:4778:0:99:0.98:1.00:0.07:0.14), (Persecutory.delusion:7:4779:0:99:0.98:1.00:0.07:0.12), (Allergic.cough:6:4780:0:99:0.98:1.00:0.06:0.11), (El ectrocardiogram.ST.segment.elevation:5:4781:0:99:0.98:1.00:0.05:0.09), (Hemia.pain:5:4781:0:9 9:0.98:1.00:0.05:0.09), (High.density.lipoprotein:5:4781:0:99:0.98:1.00:0.05:0.09), (Pertussis:5:4 781:0:99:0.98:1.00:0.05:0.09), (Delirium.tremens:4:4782:0:99:0.98:1.00:0.04:0.07), (Renal.replac ement.therapy:3:4783:0:99:0.98:1.00:0.03:0.06), (Richter's.syndrome:21:4764:0:100:0.98:1.00:0. 17:0.30), (Lower.gastrointestinal.haemorrhage:20:4765:0:100:0.98:1.00:0.17:0.29), (Mastoiditis:1 5:4770:0:100:0.98:1.00:0.13:0.23), (Lymphocyte.count.decreased:13:4772:0:100:0.98:1.00:0.12: 0.21), (Conduct.disorder:11:4774:0:100:0.98:1.00:0.10:0.18), (Ureteral.disorder:11:4774:0:100:0. 98:1.00:0.10:0.18), (Dysstasia:10:4775:0:100:0.98:1.00:0.09:0.17), (Sinusitis.bacterial:9:4776:0:1 00:0.98:1.00:0.08:0.15), (Stress.cardiomyopathy:8:4777:0:100:0.98:1.00:0.07:0.14), (Drug.detoxif ication:7:4778:0:100:0.98:1.00:0.07:0.12), (Lung.squamous.cell.carcinoma.stage.III:7:4778:0:10 0:0.98:1.00:0.07:0.12), (Diabetic.ketoacidosis:6:4779:0:100:0.98:1.00:0.06:0.11), (Oxygen.therap y:6:4779:0:100:0.98:1.00:0.06:0.11), (Dry.throat:5:4780:0:100:0.98:1.00:0.05:0.09), (Heria.repai r:5:4780:0:100:0.98:1.00:0.05:0.09), (B-cell.lymphoma.stage.IV:26:4758:0:101:0.98:1.00:0.20:0.34), (Chemotherapy.toxicity.attenuation: 24:4760:0:101:0.98:1.00:0.19:0.32), (Haemarthrosis:17:4767:0:101:0.98:1.00:0.14:0.25), (Renal.o steodystrophy:17:4767:0:101:0.98:1.00:0.14:0.25), (Blood.alkaline.phosphatase.increased:13:477 1:0:101:0.98:1.00:0.11:0.20), (Dermatitis.exfoliative:13:4771:0:101:0.98:1.00:0.11:0.20), (Cerebr al.artery.occlusion:11:4773:0:101:0.98:1.00:0.10:0.18), (Urinary.tract.infection.pseudomonal:10: 4774:0:101:0.98:1.00:0.09:0.17), (Dermatosis:9:4775:0:101:0.98:1.00:0.08:0.15), (Vasoplegia.syn drome:9:4775:0:101:0.98:1.00:0.08:0.15), (Eosinophilic.pneumonia:8:4776:0:101:0.98:1.00:0.07: 0.14), (Neurodegenerative.disorder:8:4776:0:101:0.98:1.00:0.07:0.14), (Urine.output.increased:8: 4776:0:101:0.98:1.00:0.07:0.14), (Blood.parathyroid.hormone.decreased:6:4778:0:101:0.98:1.00: 0.06:0.11), (Hyperpyrexia:6:4778:0:101:0.98:1.00:0.06:0.11), (Neurotransmitter.level.altered:6:47 78:0:101:0.98:1.00:0.06:0.11), (Urogenital.disorder:6:4778:0:101:0.98:1.00:0.06:0.11), (Drug.abu ser:5:4779:0:101:0.98:1.00:0.05:0.09), (Head.titubation:5:4779:0:101:0.98:1.00:0.05:0.09), (Osteo myelitis.acute:3:4781:0:101:0.98:1.00:0.03:0.06), (Tympanic.membrane.perforation:1:4783:0:10 1:0.98:1.00:0.01:0.02), (Undifferentiated.connective.tissue.disease:24:4759:0:102:0.98:1.00:0.19: 0.32), (Metastases.to.skin:20:4763:0:102:0.98:1.00:0.16:0.28), (Prolactin-producing.pituitary.tumour:17:4766:0:102:0.98:1.00:0.14:0.25), (Protein.C.deficiency:16:4767:0: 102:0.98:1.00:0.14:0.24), (Toxic.neuropathy:10:4773:0:102:0.98:1.00:0.09:0.16), (Vasodilation.pr ocedure:10:4773:0:102:0.98:1.00:0.09:0.16), (Metastatic.bronchial.carcinoma:9:4774:0:102:0.98: 1.00:0.08:0.15), (Thoracic.outlet.syndrome:8:4775:0:102:0.98:1.00:0.07:0.14), (Embolism.arterial: 7:4776:0:102:0.98:1.00:0.06:0.12), (Post.polio.syndrome:7:4776:0:102:0.98:1.00:0.06:0.12), (Ac cidental.exposure.to.product.by.child:6:4777:0:102:0.98:1.00:0.06:0.11), (X-ray.with.contrast:6:4777:0:102:0.98:1.00:0.06:0.11), (Cerebral.arteriosclerosis:5:4778:0:102:0.98: 1.00:0.05:0.09), (Airway.secretion.clearance.therapy:2:4781:0:102:0.98:1.00:0.02:0.04), (Surgical. preconditioning:35:4747:0:103:0.98:1.00:0.25:0.40), (Neoplasm.recurrence:27:4755:0:103:0.98: 1.00:0.21:0.34), (Ovulation.induction:25:4757:0:103:0.98:1.00:0.20:0.33), (Acute.graft.versus.hos t.disease.in.intestine:22:4760:0:103:0.98:1.00:0.18:0.30), (Bladder.neoplasm:18:4764:1:102:0.98: 0.95:0.15:0.26), (Prurigo:13:4769:0:103:0.98:1.00:0.11:0.20), (Malignant.ascites:11:4771:0:103:0 0.98:1.00:0.10:0.18), (Rheumatic.fever:11:4771:0:103:0.98:1.00:0.10:0.18), (African.trypanosomia sis:10:4772:0:103:0.98:1.00:0.09:0.16), (Pneumonia.mycoplasmal:8:4774:0:103:0.98:1.00:0.07:0 0.13), (Respiratory.tract.inflammation:8:4774:0:103:0.98:1.00:0.07:0.13), (Post.procedural.haemorr hage:6:4776:0:103:0.98:1.00:0.06:0.10), (Prostate.examination.abnormal:6:4776:0:103:0.98:1.00: 0.06:0.10), (Adenomatous.polyposis.coli:5:4777:0:103:0.98:1.00:0.05:0.09), (Neonatal.respiratory.distress.syndrome:5:4777:0:103:0.98:1.00:0.05:0.09), (Refractory.anaemia.with.ringed.siderobla sts:5:4777:0:103:0.98:1.00:0.05:0.09), (Burning.feet.syndrome:4:4778:0:103:0.98:1.00:0.04:0.07), (Sinus.bradycardia:4:4778:0:103:0.98:1.00:0.04:0.07), (Matemal.exposure.during.pregnancy:37: 4744:0:104:0.98:1.00:0.26:0.42), (Groin.abscess:19:4762:1:103:0.98:0.95:0.16:0.27), (Guttate.pso riasis:13:4768:0:104:0.98:1.00:0.11:0.20), (Leprosy:11:4770:0:104:0.98:1.00:0.10:0.17), (Oropha ryngeal.squamous.cell.carcinoma:9:4772:0:104:0.98:1.00:0.08:0.15), (Reproductive.tract.disorder: 8:4773:0:104:0.98:1.00:0.07:0.13), (Femoral.neck.fracture:7:4774:0:104:0.98:1.00:0.06:0.12), (B ladder.prolapse:4:4777:0:104:0.98:1.00:0.04:0.07), (Electrocardiogram.abnormal:3:4778:0:104:0. 98:1.00:0.03:0.05), (Cytogenetic.abnormality:19:4761:0:105:0.98:1.00:0.15:0.27), (Vasopressive.t herapy:19:4761:3:102:0.98:0.86:0.16:0.27), (Mantle.cell.lymphoma.recurrent:16:4764:0:105:0.98: 1.00:0.13:0.23), (Adrenocortical.insufficiency.acute:13:4767:1:104:0.98:0.93:0.11:0.20), (Oesop hageal.varices.haemorrhage:13:4767:0:105:0.98:1.00:0.11:0.20), (Gastrectomy:11:4769:0:105:0. 98:1.00:0.09:0.17), (Ventricular.dysfunction:11:4769:0:105:0.98:1.00:0.09:0.17), (Retching:10:47 70:0:105:0.98:1.00:0.09:0.16), (Traumatic.lung.injury:10:4770:0:105:0.98:1.00:0.09:0.16), (Perip heral.artery.thrombosis:8:4772:0:105:0.98:1.00:0.07:0.13), (Drug.administration.error:7:4773:0:1 05:0.98:1.00:0.06:0.12), (Blood.urine.present:6:4774:0:105:0.98:1.00:0.05:0.10), (Schizophrenia,. undifferentiated.type:5:4775:0:105:0.98:1.00:0.05:0.09), (Diabetic.autonomic.neuropathy:4:4776: 0:105:0.98:1.00:0.04:0.07), (Jaw.disorder:4:4776:0:105:0.98:1.00:0.04:0.07), (Blood.triglycerides. decreased:2:4778:0:105:0.98:1.00:0.02:0.04), (Intervertebral.disc.displacement:2:4778:0:105:0.9 8:1.00:0.02:0.04), (Small.cell.lung.cancer.metastatic:16:4763:0:106:0.98:1.00:0.13:0.23), (Blood. prolactin.increased:12:4767:0:106:0.98:1.00:0.10:0.18), (Hydronephrosis:12:4767:0:106:0.98:1.0 0:0.10:0.18), (Papilloedema:11:4768:0:106:0.98:1.00:0.09:0.17), (Radioactive.iodine.therapy:11:4 768:0:106:0.98:1.00:0.09:0.17), (Blood.electrolytes:9:4770:0:106:0.98:1.00:0.08:0.15), (Comeal.a brasion:9:4770:0:106:0.98:1.00:0.08:0.15), (Radius.fracture:8:4771:0:106:0.98:1.00:0.07:0.13), (T endon.disorder:7:4772:0:106:0.98:1.00:0.06:0.12), (Angiodysplasia:5:4774:0:106:0.98:1.00:0.05: 0.09), (Diabetic.complication:4:4775:0:106:0.98:1.00:0.04:0.07), (Osteomyelitis.chronic:4:4775:0: 106:0.98:1.00:0.04:0.07), (Non-Hodgkin's.lymphoma.recurrent:31:4747:0:107:0.98:1.00:0.22:0.37), (Glycosylated.haemoglobin: 16:4762:0:107:0.98:1.00:0.13:0.23), (Nasal.sinus.cancer:15:4763:0:107:0.98:1.00:0.12:0.22), (Ca pillary.leak.syndrome:14:4764:0:107:0.98:1.00:0.12:0.21), (Arachnoiditis:13:4765:0:107:0.98:1.0 0:0.11:0.20), (Hyperplasia:11:4767:0:107:0.98:1.00:0.09:0.17), (Abnormal.clotting.factor:10:4768: 0:107:0.98:1.00:0.09:0.16), (Neuroleptic.malignant.syndrome:10:4768:0:107:0.98:1.00:0.09:0.16), (Product.substitution:8:4770:0:107:0.98:1.00:0.07:0.13), (Multifocal.motor.neuropathy:7:4771: 0:107:0.98:1.00:0.06:0.12), (Gallbladder.operation:6:4772:0:107:0.98:1.00:0.05:0.10), (Oesophag eal.stenosis:6:4772:0:107:0.98:1.00:0.05:0.10), (Dermal.cyst:5:4773:0:107:0.98:1.00:0.04:0.09), (Cerumen.impaction:4:4774:0:107:0.98:1.00:0.04:0.07), (Cystoscopy:4:4774:0:107:0.98:1.00:0.04: 0.07), (Ovarian.failure:19:4758:0:108:0.98:1.00:0.15:0.26), (Ureteric.cancer:10:4767:0:108:0.98: 1.00:0.08:0.16), (Obstruction:6:4771:0:108:0.98:1.00:0.05:0.10), (Organic.erectile.dysfunction:6: 4771:0:108:0.98:1.00:0.05:0.10), (Mechanical.ventilation:5:4772:0:108:0.98:1.00:0.04:0.08), (All ergy.to.plants:4:4773:0:108:0.98:1.00:0.04:0.07), (Mycobacterium.tuberculosis.complex.test.posi tive:17:4759:0:109:0.98:1.00:0.13:0.24), (Leiomyosarcoma.metastatic:14:4762:0:109:0.98:1.00:0 0.11:0.20), (Parathyroidectomy:14:4762:0:109:0.98:1.00:0.11:0.20), (Asthmatic.crisis:13:4763:0:1 09:0.98:1.00:0.11:0.19), (Secondary.hypogonadism:13:4763:0:109:0.98:1.00:0.11:0.19), (Dysfunc tional.uterine.bleeding:10:4766:0:109:0.98:1.00:0.08:0.16), (Normal.tension.glaucoma:6:4770:0: 109:0.98:1.00:0.05:0.10), (Lumbar.vertebral.fracture:5:4771:0:109:0.98:1.00:0.04:0.08), (Bundle. branch.block.left:3:4773:0:109:0.98:1.00:0.03:0.05), (Anaesthesia.reversal:19:4756:0:110:0.98:1. 00:0.15:0.26), (Ovarian.cancer.stage.IV:17:4758:0:110:0.98:1.00:0.13:0.24), (Mastitis:16:4759:0: 110:0.98:1.00:0.13:0.23), (Adrenocorticotropic.hormone.deficiency:13:4762:1:109:0.98:0.93:0.1 1:0.19), (Cervix.cancer.metastatic:13:4762:0:110:0.98:1.00:0.11:0.19), (Paraparesis:11:4764:0:11 0:0.98:1.00:0.09:0.17), (Methylenetetrahydrofolate.reductase.deficiency:9:4766:0:110:0.98:1.00: 0.08:0.14), (Bone.scan:8:4767:0:110:0.98:1.00:0.07:0.13), (Pelvic.fracture:7:4768:0:110:0.98:1.0 0:0.06:0.11), (Trigger.finger:7:4768:0:110:0.98:1.00:0.06:0.11), (Faecal.incontinence:6:4769:0:11 0:0.98:1.00:0.05:0.10), (Psychotic.behaviour:6:4769:0:110:0.98:1.00:0.05:0.10), (Tachycardia.par oxysmal:6:4769:0:110:0.98:1.00:0.05:0.10), (Therapy.cessation:5:4770:0:110:0.98:1.00:0.04:0.0 8), (Rales:4:4771:0:110:0.98:1.00:0.04:0.07), (Behavioural.and.psychiatric.symptoms.of.dementia: 2:4773:0:110:0.98:1.00:0.02:0.04), (Dysaesthesia:22:4752:0:111:0.98:1.00:0.17:0.28), (Metastas es.to.nervous.system:15:4759:0:111:0.98:1.00:0.12:0.21), (BRAF.gene.mutation:13:4761:0:111:0 0.98:1.00:0.10:0.19), (Blood.osmolarity.decreased:12:4762:0:111:0.98:1.00:0.10:0.18), (Meningoe ncephalitis.herpetic:12:4762:0:111:0.98:1.00:0.10:0.18), (Synovial.cyst:12:4762:0:111:0.98:1.00: 0.10:0.18), (Herpes.zoster.disseminated:11:4763:0:111:0.98:1.00:0.09:0.17), (Procedural.vomiting: 11:4763:0:111:0.98:1.00:0.09:0.17), (Endophthalmitis:9:4765:0:111:0.98:1.00:0.08:0.14), (Gener al.physical.condition.abnormal:8:4766:0:111:0.98:1.00:0.07:0.13), (Allergic.bronchitis:7:4767:0: 111:0.98:1.00:0.06:0.11), (Lip.dry:6:4768:0:111:0.98:1.00:0.05:0.10), (Chapped.lips:5:4769:0:11 1:0.98:1.00:0.04:0.08), (Psoas.abscess:5:4769:0:111:0.98:1.00:0.04:0.08), (Gastrostomy:4:4770:0: 111:0.98:1.00:0.03:0.07), (White.blood.cell.count.abnormal:20:4753:0:112:0.98:1.00:0.15:0.26), (Plasmapheresis:17:4756:0:112:0.98:1.00:0.13:0.23), (Blood.parathyroid.hormone.increased:14:4 759:0:112:0.98:1.00:0.11:0.20), (Gastrointestinal.toxicity:13:4760:0:112:0.98:1.00:0.10:0.19), (H epatobiliary.cancer:13:4760:0:112:0.98:1.00:0.10:0.19), (Cerebral.thrombosis:11:4762:0:112:0.9 8:1.00:0.09:0.16), (Social.phobia:11:4762:0:112:0.98:1.00:0.09:0.16), (Drug.level.increased:10:47 63:0:112:0.98:1.00:0.08:0.15), (Drug.tolerance:10:4763:0:112:0.98:1.00:0.08:0.15), (Mood.disord er.due.to.a.general.medical.condition:9:4764:0:112:0.98:1.00:0.07:0.14), (Allergic.respiratory.dis ease:7:4766:0:112:0.98:1.00:0.06:0.11), (Libido.disorder:6:4767:0:112:0.98:1.00:0.05:0.10), (Inte rvertebral.disc.compression:3:4770:0:112:0.98:1.00:0.03:0.05), (Exposure.during.breast.feeding: 21:4751:0:113:0.98:1.00:0.16:0.27), (SLE.arthritis:19:4753:0:113:0.98:1.00:0.14:0.25), (Catatonia: 16:4756:0:113:0.98:1.00:0.12:0.22), (Blood.bilirubin.increased:15:4757:0:113:0.98:1.00:0.12:0. 21), (Pancreatic.carcinoma.stage.IV:14:4758:0:113:0.98:1.00:0.11:0.20), (Abdominal.hernia:11:4 761:0:113:0.98:1.00:0.09:0.16), (Chronic.left.ventricular.failure:9:4763:0:113:0.98:1.00:0.07:0.1 4), (Gastrointestinal.tract.irritation:9:4763:0:113:0.98:1.00:0.07:0.14), (Increased.viscosity.of.bron chial.secretion:9:4763:0:113:0.98:1.00:0.07:0.14), (Clostridium.test.positive:8:4764:0:113:0.98:1. 00:0.07:0.12), (Gastric.cancer.recurrent:7:4765:0:113:0.98:1.00:0.06:0.11), (Activated.partial.thro mboplastin.time.prolonged:6:4766:0:113:0.98:1.00:0.05:0.10), (Heart.valve.operation:6:4766:0:1 13:0.98:1.00:0.05:0.10), (Rhinitis.perennial:5:4767:0:113:0.98:1.00:0.04:0.08), (Congenital.neuro pathy:4:4768:0:113:0.98:1.00:0.03:0.07), (Speech.disorder:19:4752:0:114:0.98:1.00:0.14:0.25), (Allergic.granulomatous.angiitis:18:4753:0:114:0.98:1.00:0.14:0.24), (Hyperprolactinaemia:16:47 55:0:114:0.98:1.00:0.12:0.22), (Bone.marrow.transplant.rejection:13:4758:0:114:0.98:1.00:0.10:0 0.19), (Tuberculin.test:13:4758:0:114:0.98:1.00:0.10:0.19), (Beta.haemolytic.streptococcal.infectio n:11:4760:0:114:0.98:1.00:0.09:0.16), (Blood.thyroid.stimulating.hormone:9:4762:0:114:0.98:1.0 0:0.07:0.14), (Medication.error:9:4762:0:114:0.98:1.00:0.07:0.14), (Coagulation.time.shortened:8: 4763:0:114:0.98:1.00:0.07:0.12), (Prader-Willi.syndrome:7:4764:0:114:0.98:1.00:0.06:0.11), (Blood.viscosity.increased:5:4766:0:114:0.98: 1.00:0.04:0.08), (Familial.tremor:4:4767:0:114:0.98:1.00:0.03:0.07), (Follicular.thyroid.cancer:4: 4767:0:114:0.98:1.00:0.03:0.07), (Continuous.positive.airway.pressure:3:4768:0:114:0.98:1.00:0. 03:0.05), (Renal.injury:21:4749:0:115:0.98:1.00:0.15:0.27), (Altered.state.of.consciousness:18:47 52:0:115:0.98:1.00:0.14:0.24), (Central.nervous.system.lupus:17:4753:0:115:0.98:1.00:0.13:0.23), (Calcinosis:16:4754:0:115:0.98:1.00:0.12:0.22), (Left.ventricular.hypertrophy:12:4758:0:115:0.9 8:1.00:0.09:0.17), (Adenomyosis:11:4759:0:115:0.98:1.00:0.09:0.16), (Poisoning:9:4761:0:115:0. 98:1.00:0.07:0.14), (Self-medication:8:4762:0:115:0.98:1.00:0.07:0.12), (Mineral.metabolism.disorder:6:4764:0:115:0.98: 1.00:0.05:0.09), (Sciatic.nerve.neuropathy:6:4764:0:115:0.98:1.00:0.05:0.09), (Orthostatic.intoler ance:5:4765:0:115:0.98:1.00:0.04:0.08), (Joint.dislocation:4:4766:0:115:0.98:1.00:0.03:0.07), (Po st-traumatic.pain:4:4766:0:115:0.98:1.00:0.03:0.07), (In.vitro.fertilisation:29:4740:0:116:0.98:1.00: 0.20:0.33), (Viral.hepatitis.carrier:27:4742:0:116:0.98:1.00:0.19:0.32), (Acute.myelomonocytic.le ukaemia:23:4746:0:116:0.98:1.00:0.17:0.28), (Thymoma.malignant:22:4747:0:116:0.98:1.00:0.1 6:0.28), (Anticoagulation.drug.level:19:4750:0:116:0.98:1.00:0.14:0.25), (Progressive.multiple.scl erosis:16:4753:0:116:0.98:1.00:0.12:0.22), (Visual.acuity.reduced:15:4754:0:116:0.98:1.00:0.11: 0.21), (Gastric.polyps:14:4755:0:116:0.98:1.00:0.11:0.19), (Intestinal.haemorrhage:12:4757:0:116: 0.98:1.00:0.09:0.17), (Blood.electrolytes.abnormal:10:4759:0:116:0.98:1.00:0.08:0.15), (Erythro poiesis.abnormal:9:4760:0:116:0.98:1.00:0.07:0.13), (H1N1.influenza:9:4760:0:116:0.98:1.00:0. 07:0.13), (Hyperventilation:9:4760:0:116:0.98:1.00:0.07:0.13), (Lung.carcinoma.cell.type.unspeci fied.stage.I:9:4760:0:116:0.98:1.00:0.07:0.13), (Gouty.tophus:7:4762:0:116:0.98:1.00:0.06:0.11), (Seborrhoeic.keratosis:7:4762:0:116:0.98:1.00:0.06:0.11), (Wrist.fracture:6:4763:0:116:0.98:1.00: 0.05:0.09), (Platelet.count:4:4765:0:116:0.98:1.00:0.03:0.06), (Pneumococcal.immunisation:4:47 65:0:116:0.98:1.00:0.03:0.06), (Mixed.dementia:2:4767:0:116:0.98:1.00:0.02:0.03), (Poisoning.de liberate:0:4768:0:117:0.98:#NUM!:0.00:#NUM!), (Dermatitis.bullous:24:4744:1:116:0.98:0.96:0 0.17:0.29), (Disorientation:13:4755:0:117:0.98:1.00:0.10:0.18), (Kawasaki's.disease:13:4755:0:117:0.98:1.00:0.10:0.18), (Haemorrhoidal.haemorrhage:12:4756:0:117:0.98:1.00:0.09:0.17), (Cyclic. vomiting.syndrome:11:4757:0:117:0.98:1.00:0.09:0.16), (Fusarium.infection:11:4757:0:117:0.98: 1.00:0.09:0.16), (Gingival.bleeding:8:4760:0:117:0.98:1.00:0.06:0.12), (Burning.mouth.syndrome: 6:4762:0:117:0.98:1.00:0.05:0.09), (Agitated.depression:4:4764:0:117:0.98:1.00:0.03:0.06), (Ang le.closure.glaucoma:1:4767:0:117:0.98:1.00:0.01:0.02), (Bone.decalcification:17:4750:0:118:0.9 8:1.00:0.13:0.22), (Osteomalacia:17:4750:0:118:0.98:1.00:0.13:0.22), (Aortic.valve.disease:16:47 51:0:118:0.98:1.00:0.12:0.21), (Implantable.defibrillator.insertion:13:4754:0:118:0.98:1.00:0.10: 0.18), (Blood.count:12:4755:0:118:0.98:1.00:0.09:0.17), (Comeal.deposits:11:4756:0:118:0.98:1. 00:0.09:0.16), (Accidental.overdose:10:4757:0:118:0.98:1.00:0.08:0.14), (Arthrodesis:10:4757:0: 118:0.98:1.00:0.08:0.14), (Hepatic.pain:10:4757:0:118:0.98:1.00:0.08:0.14), (Sunbum:10:4757:0: 118:0.98:1.00:0.08:0.14), (Biliary.colic:9:4758:0:118:0.98:1.00:0.07:0.13), (Catheter.site.infection: 9:4758:0:118:0.98:1.00:0.07:0.13), (Muckle-Wells.syndrome:8:4759:0:118:0.98:1.00:0.06:0.12), (Neck.surgery:8:4759:0:118:0.98:1.00:0.06: 0.12), (Hypoacusis:7:4760:0:118:0.98:1.00:0.06:0.11), (Anaphylaxis.treatment:6:4761:0:118:0.98: 1.00:0.05:0.09), (Faecaloma:6:4761:0:118:0.98:1.00:0.05:0.09), (Pathogen.resistance:5:4762:0:11 8:0.98:1.00:0.04:0.08), (Skin.bacterial.infection:5:4762:0:118:0.98:1.00:0.04:0.08), (Body.temper ature.abnormal:3:4764:0:118:0.98:1.00:0.02:0.05), (Vasoconstriction:21:4745:0:119:0.98:1.00:0. 15:0.26), (Metastases.to.breast:15:4751:0:119:0.98:1.00:0.11:0.20), (Spinal.column.injury:14:475 2:0:119:0.98:1.00:0.11:0.19), (Antithrombin.III.decreased:10:4756:0:119:0.98:1.00:0.08:0.14), (C ystic.fibrosis.related.diabetes:10:4756:0:119:0.98:1.00:0.08:0.14), (Gangrene:8:4758:0:119:0.98: 1.00:0.06:0.12), (Nail.growth.abnormal:8:4758:0:119:0.98:1.00:0.06:0.12), (Serotonin.syndrome: 8:4758:0:119:0.98:1.00:0.06:0.12), (Concussion:7:4759:0:119:0.98:1.00:0.06:0.11), (Anal.inconti nence:6:4760:0:119:0.98:1.00:0.05:0.09), (Vaginal.discharge:6:4760:0:119:0.98:1.00:0.05:0.09), (Knee.deformity:5:4761:0:119:0.98:1.00:0.04:0.08), (Spinal.muscular.atrophy:5:4761:0:119:0.98: 1.00:0.04:0.08), (Cystitis.haemorrhagic:26:4739:0:120:0.98:1.00:0.18:0.30), (Encephalitis.autoim mune:26:4739:0:120:0.98:1.00:0.18:0.30), (Pneumonia.cytomegaloviral:26:4739:0:120:0.98:1.00: 0.18:0.30), (Rhabdomyosarcoma:21:4744:0:120:0.98:1.00:0.15:0.26), (Drug.reaction.with.eosino philia.and.systemic.symptoms:20:4745:1:119:0.98:0.95:0.14:0.25), (Uterine.contractions.during.p regnancy:18:4747:0:120:0.98:1.00:0.13:0.23), (Alveolitis.allergic:16:4749:0:120:0.98:1.00:0.12:0 0.21), (Orthostatic.hypertension:10:4755:0:120:0.98:1.00:0.08:0.14), (Injection.site.pruritus:8:4757: 0:120:0.98:1.00:0.06:0.12), (Arterial.stent.insertion:6:4759:0:120:0.98:1.00:0.05:0.09), (Ocular.r osacea:5:4760:0:120:0.98:1.00:0.04:0.08), (Dermo-hypodermitis:2:4763:0:120:0.98:1.00:0.02:0.03), (Central.nervous.system.lesion:21:4743:0:121:0 0.98:1.00:0.15:0.26), (Serum.ferritin.increased:19:4745:0:121:0.98:1.00:0.14:0.24), (Aspergilloma: 18:4746:0:121:0.98:1.00:0.13:0.23), (White.blood.cell.count:17:4747:0:121:0.98:1.00:0.12:0.22), (Malignant.neoplasm.of.thymus:14:4750:0:121:0.98:1.00:0.10:0.19), (Chronic.leukaemia:11:475 3:0:121:0.98:1.00:0.08:0.15), (Iodine.deficiency:10:4754:0:121:0.98:1.00:0.08:0.14), (Pharyngeal. disorder:10:4754:0:121:0.98:1.00:0.08:0.14), (Liver.function.test.increased:9:4755:0:121:0.98:1. 00:0.07:0.13), (Primary.hyperaldosteronism:9:4755:0:121:0.98:1.00:0.07:0.13), (Substance.use.di sorder:9:4755:0:121:0.98:1.00:0.07:0.13), (Thyroiditis.chronic:9:4755:0:121:0.98:1.00:0.07:0.13), (Cerebral.artery.embolism:7:4757:0:121:0.98:1.00:0.05:0.10), (Phobia:6:4758:0:121:0.98:1.00:0. 05:0.09), (Phaeochromocytoma:4:4760:0:121:0.98:1.00:0.03:0.06), (Sleep.phase.rhythm.disturban ce:3:4761:0:121:0.98:1.00:0.02:0.05), (Haematotoxicity:20:4743:0:122:0.98:1.00:0.14:0.25), (Inte rnational.normalised.ratio.abnormal:17:4746:0:122:0.98:1.00:0.12:0.22), (Benign.intracranial.hyp ertension:14:4749:0:122:0.98:1.00:0.10:0.19), (Breast.cancer.stage.I:14:4749:0:122:0.98:1.00:0.1 0:0.19), (Rash.papular:13:4750:0:122:0.98:1.00:0.10:0.18), (Epidural.analgesia:9:4754:0:122:0.98: 1.00:0.07:0.13), (Polypoidal.choroidal.vasculopathy:9:4754:0:122:0.98:1.00:0.07:0.13), (Atrophy: 7:4756:0:122:0.98:1.00:0.05:0.10), (Brain.cancer.metastatic:7:4756:0:122:0.98:1.00:0.05:0.10), (Carcinoid.tumour.pulmonary:7:4756:0:122:0.98:1.00:0.05:0.10), (Thyroxine.decreased:7:4756:0: 122:0.98:1.00:0.05:0.10), (Hearing.impaired:4:4759:0:122:0.98:1.00:0.03:0.06), (Blood.test.abnor mal:18:4744:0:123:0.97:1.00:0.13:0.23), (Pneumococcal.infection:15:4747:0:123:0.97:1.00:0.11: 0.20), (Staphylococcus.test.positive:15:4747:0:123:0.97:1.00:0.11:0.20), (Intestinal.resection:14:4 748:0:123:0.97:1.00:0.10:0.19), (Lung.carcinoma.cell.type.unspecified.recurrent:14:4748:0:123:0 0.97:1.00:0.10:0.19), (Stiff.person.syndrome:14:4748:0:123:0.97:1.00:0.10:0.19), (Cardiogenic.sho ek:13:4749:0:123:0.97:1.00:0.10:0.17), (Retinal.detachment:13:4749:0:123:0.97:1.00:0.10:0.17), (Corneal.disorder:12:4750:0:123:0.97:1.00:0.09:0.16), (Lung.squamous.cell.carcinoma.metastatic: 9:4753:0:123:0.97:1.00:0.07:0.13), (Chronic.disease:8:4754:0:123:0.97:1.00:0.06:0.12), (Dermatit is.psoriasiform:8:4754:0:123:0.97:1.00:0.06:0.12), (Hemiplegia:8:4754:0:123:0.97:1.00:0.06:0.1 2), (Xeroderma:8:4754:0:123:0.97:1.00:0.06:0.12), (Alcoholic.liver.disease:7:4755:0:123:0.97:1.0 0:0.05:0.10), (Prostatectomy:7:4755:0:123:0.97:1.00:0.05:0.10), (Respiratory.symptom:7:4755:0: 123:0.97:1.00:0.05:0.10), (Blood.viscosity.abnormal:6:4756:0:123:0.97:1.00:0.05:0.09), (Snake.bi te:6:4756:0:123:0.97:1.00:0.05:0.09), (Nasal.pruritus:5:4757:0:123:0.97:1.00:0.04:0.08), (Simple. partial.seizures:23:4738:0:124:0.97:1.00:0.16:0.27), (Neuromyelitis.optica:21:4740:0:124:0.97:1. 00:0.14:0.25), (Asbestosis:9:4752:0:124:0.97:1.00:0.07:0.13), (Foot.fracture:9:4752:0:124:0.97:1. 00:0.07:0.13), (Muscle.atrophy:7:4754:0:124:0.97:1.00:0.05:0.10), (Pharyngeal.oedema:7:4754:0: 124:0.97:1.00:0.05:0.10), (Vitamin.B1.decreased:7:4754:0:124:0.97:1.00:0.05:0.10), (Bronchitis.b acterial:5:4756:0:124:0.97:1.00:0.04:0.07), (Anal.abscess:21:4739:0:125:0.97:1.00:0.14:0.25), (H ereditary.haemorrhagic.telangiectasia:13:4747:0:125:0.97:1.00:0.09:0.17), (Mediastinitis:13:4747: 0:125:0.97:1.00:0.09:0.17), (Bronchopulmonary.dysplasia:10:4750:0:125:0.97:1.00:0.07:0.14), (Middle.ear.effusion:10:4750:0:125:0.97:1.00:0.07:0.14), (Bone.metabolism.disorder:7:4753:0:12 5:0.97:1.00:0.05:0.10), (Metabolic.surgery:7:4753:0:125:0.97:1.00:0.05:0.10), (Weight:4:4756:0: 125:0.97:1.00:0.03:0.06), (Recurrent.cancer:31:4728:0:126:0.97:1.00:0.20:0.33), (Precursor.B-lymphoblastic.lymphoma:30:4729:0:126:0.97:1.00:0.19:0.32), (Familial.mediterranean.fever:22: 4737:0:126:0.97:1.00:0.15:0.26), (Polyarteritis.nodosa:20:4739:0:126:0.97:1.00:0.14:0.24), (BK.v irus.infection:19:4740:0:126:0.97:1.00:0.13:0.23), (Feeling.jittery:14:4745:0:126:0.97:1.00:0.10: 0.18), (Cyanosis:12:4747:0:126:0.97:1.00:0.09:0.16), (House.dust.allergy:10:4749:0:126:0.97:1.0 0:0.07:0.14), (Botulism:7:4752:0:126:0.97:1.00:0.05:0.10), (Pulmonary.sepsis:6:4753:0:126:0.97: 1.00:0.05:0.09), (Ornithine.transcarbamoylase.deficiency:4:4755:0:126:0.97:1.00:0.03:0.06), (Dis seminated.tuberculosis:50:4708:1:126:0.97:0.98:0.28:0.44), (Immunoglobulin.therapy:24:4734:0: 127:0.97:1.00:0.16:0.27), (Lymph.node.tuberculosis:22:4736:0:127:0.97:1.00:0.15:0.26), (Lung.a denocarcinoma.stage.III:18:4740:0:127:0.97:1.00:0.12:0.22), (Pre-existing.disease:15:4743:0:127:0.97:1.00:0.11:0.19), (Vitamin.B12.abnormal:11:4747:0:127:0.97: 1.00:0.08:0.15), (Skin.hyperpigmentation:10:4748:0:127:0.97:1.00:0.07:0.14), (Aspiration.pleural. cavity:4:4754:0:127:0.97:1.00:0.03:0.06), (Adrenocortical.steroid.therapy:26:4731:0:128:0.97:1 0.00:0.17:0.29), (Langerhans′.cell.histiocytosis:26:4731:0:128:0.97:1.00:0.17:0.29), (Amino.acid.m etabolism.disorder:16:4741:0:128:0.97:1.00:0.11:0.20), (Colorectal.cancer.stage.IV:16:4741:0:12 8:0.97:1.00:0.11:0.20), (Calcium.metabolism.disorder:15:4742:0:128:0.97:1.00:0.10:0.19), (Haem oglobin:15:4742:0:128:0.97:1.00:0.10:0.19), (Humerus.fracture:14:4743:0:128:0.97:1.00:0.10:0.1 8), (Muscular.dystrophy:14:4743:0:128:0.97:1.00:0.10:0.18), (Dental.cleaning:11:4746:0:128:0.9 7:1.00:0.08:0.15), (Aortic.valve.stenosis:10:4747:0:128:0.97:1.00:0.07:0.14), (Central.nervous.system. stimulation:10:4747:0:128:0.97:1.00:0.07:0.14), (Prophylaxis.against.bronchospasm:10:4747: 0:128:0.97:1.00:0.07:0.14), (Capillary.disorder:8:4749:0:128:0.97:1.00:0.06:0.11), (Radiation.pn eumonitis:8:4749:0:128:0.97:1.00:0.06:0.11), (Feeling.hot:7:4750:0:128:0.97:1.00:0.05:0.10), (Ga strinoma:5:4752:0:128:0.97:1.00:0.04:0.07), (Enterocolitis.haemorrhagic:22:4734:0:129:0.97:1.0 0:0.15:0.25), (Varicella:17:4739:0:129:0.97:1.00:0.12:0.21), (Enterococcal.bacteraemia:14:4742:0: 129:0.97:1.00:0.10:0.18), (International.normalised.ratio:13:4743:0:129:0.97:1.00:0.09:0.17), (Multi-vitamin.deficiency:13:4743:0:129:0.97:1.00:0.09:0.17), (Infusion.site.extravasation:9:4747:0:129: 0.97:1.00:0.07:0.12), (Selective.IgM.immunodeficiency:5:4751:0:129:0.97:1.00:0.04:0.07), (Lip. swelling:4:4752:0:129:0.97:1.00:0.03:0.06), (Lymphoproliferative.disorder:31:4724:0:130:0.97:1 0.00:0.19:0.32), (Myocarditis:30:4725:1:129:0.97:0.97:0.19:0.32), (Uterine.leiomyosarcoma:18:47 37:0:130:0.97:1.00:0.12:0.22), (Protein.deficiency:16:4739:0:130:0.97:1.00:0.11:0.20), (Calculus. urinary:13:4742:0:130:0.97:1.00:0.09:0.17), (Impulse-control.disorder:13:4742:0:130:0.97:1.00:0.09:0.17), (Gastric.infection:12:4743:0:130:0.97:1.00: 0.08:0.16), (Bile.acid.malabsorption:11:4744:0:130:0.97:1.00:0.08:0.14), (Catarrh:9:4746:0:130:0 0.97:1.00:0.06:0.12), (Cirrhosis.alcoholic:9:4746:0:130:0.97:1.00:0.06:0.12), (Peripheral.nerve.inju ry:9:4746:0:130:0.97:1.00:0.06:0.12), (Bladder.catheterisation:3:4752:0:130:0.97:1.00:0.02:0.04), (Tenosynovitis:17:4737:0:131:0.97:1.00:0.11:0.21), (Myxoedema:16:4738:0:131:0.97:1.00:0.11: 0.20), (Lipohypertrophy:15:4739:0:131:0.97:1.00:0.10:0.19), (Platelet.count.abnormal:14:4740:0: 131:0.97:1.00:0.10:0.18), (Pulmonary.infarction:13:4741:0:131:0.97:1.00:0.09:0.17), (Fat.tissue.i ncreased:12:4742:0:131:0.97:1.00:0.08:0.15), (VIIth.nerve.paralysis:12:4742:0:131:0.97:1.00:0.0 8:0.15), (Melaena:11:4743:0:131:0.97:1.00:0.08:0.14), (Blood.homocysteine.increased:10:4744:0: 131:0.97:1.00:0.07:0.13), (Hepato-lenticular.degeneration:9:4745:0:131:0.97:1.00:0.06:0.12), (Mucosal.infection:9:4745:0:131:0.97: 1.00:0.06:0.12), (Psychotic.disorder.due.to.a.general.medical.condition:8:4746:0:131:0.97:1.00:0 0.06:0.11), (Therapy.change:8:4746:0:131:0.97:1.00:0.06:0.11), (Hospitalisation:6:4748:0:131:0.97: 1.00:0.04:0.08), (Secondary.hypertension:24:4729:0:132:0.97:1.00:0.15:0.27), (Abortion.induced: 19:4734:0:132:0.97:1.00:0.13:0.22), (Psychosomatic.disease:18:4735:0:132:0.97:1.00:0.12:0.21), (Hepatotoxicity:14:4739:0:132:0.97:1.00:0.10:0.18), (Malignant.pleural.effusion:14:4739:0:132: 0.97:1.00:0.10:0.18), (Medical.procedure:14:4739:0:132:0.97:1.00:0.10:0.18), (Middle.insomnia: 9:4744:0:132:0.97:1.00:0.06:0.12), (Parotitis:8:4745:0:132:0.97:1.00:0.06:0.11), (Device.issue:7:4 746:0:132:0.97:1.00:0.05:0.10), (Joint.arthroplasty:6:4747:0:132:0.97:1.00:0.04:0.08), (Device.rel ated.sepsis:4:4749:0:132:0.97:1.00:0.03:0.06), (Histoplasmosis:28:4724:1:132:0.97:0.97:0.18:0.3 0), (Post-traumatic.epilepsy:16:4736:0:133:0.97:1.00:0.11:0.19), (Lower.limb.fracture:11:4741:0:133:0.97: 1.00:0.08:0.14), (Superinfection:11:4741:0:133:0.97:1.00:0.08:0.14), (High.density.lipoprotein.in creased:10:4742:0:133:0.97:1.00:0.07:0.13), (Gaucher's.disease.type.I:9:4743:0:133:0.97:1.00:0. 06:0.12), (Inner.ear.disorder:9:4743:0:133:0.97:1.00:0.06:0.12), (Lice.infestation:9:4743:0:133:0. 97:1.00:0.06:0.12), (Antioestrogen.therapy:20:4731:0:134:0.97:1.00:0.13:0.23), (Erythrodermic.p soriasis:16:4735:0:134:0.97:1.00:0.11:0.19), (Photosensitivity.reaction:16:4735:0:134:0.97:1.00: 0.11:0.19), (Upper.limb.fracture:9:4742:0:134:0.97:1.00:0.06:0.12), (Facial.spasm:7:4744:0:134:0 0.97:1.00:0.05:0.09), (Astrocytoma:28:4722:0:135:0.97:1.00:0.17:0.29), (Ulcerative.keratitis:23:47 27:0:135:0.97:1.00:0.15:0.25), (Substance.abuse:22:4728:0:135:0.97:1.00:0.14:0.25), (Metastatic. squamous.cell.carcinoma:19:4731:0:135:0.97:1.00:0.12:0.22), (Myelitis.transverse:19:4731:0:13 5:0.97:1.00:0.12:0.22), (Perioperative.analgesia:17:4733:1:134:0.97:0.94:0.11:0.20), (Hypercalcae mia.of.malignancy:13:4737:0:135:0.97:1.00:0.09:0.16), (Tocolysis:13:4737:0:135:0.97:1.00:0.09: 0.16), (Blood.pressure.diastolic.increased:12:4738:0:135:0.97:1.00:0.08:0.15), (Diarrhoea.infecti ous:12:4738:0:135:0.97:1.00:0.08:0.15), (Endocarditis.staphylococcal:2:4748:0:135:0.97:1.00:0.0 1:0.03), (Salvage.therapy:48:4701:1:135:0.97:0.98:0.26:0.41), (Medulloblastoma:34:4715:0:136:0 0.97:1.00:0.20:0.33), (Neuroendocrine.carcinoma.metastatic:26:4723:0:136:0.97:1.00:0.16:0.28), (Primary.hypothyroidism:23:4726:0:136:0.97:1.00:0.14:0.25), (Secondary.hypothyroidism:18:473 1:0:136:0.97:1.00:0.12:0.21), (Paraplegia:15:4734:0:136:0.97:1.00:0.10:0.18), (Renal.hypertensio n:14:4735:0:136:0.97:1.00:0.09:0.17), (Tumour.necrosis.factor.receptor-associated.periodic.syndrome:14:4735:0:136:0.97:1.00:0.09:0.17), (Blood.uric.acid.decreased:13: 4736:0:136:0.97:1.00:0.09:0.16), (Pancreatic.enzyme.abnormality:10:4739:0:136:0.97:1.00:0.07: 0.13), (Ear.congestion:7:4742:0:136:0.97:1.00:0.05:0.09), (Radicular.pain:3:4746:0:136:0.97:1.00:0.02:0.04), (Scan:25:4723:0:137:0.97:1.00:0.15:0.27), (Encephalitis.viral:24:4724:1:136:0.97:0.9 6:0.15:0.26), (Intellectual.disability:23:4725:2:135:0.97:0.92:0.15:0.25), (Iridocyclitis:18:4730:0: 137:0.97:1.00:0.12:0.21), (Cancer.hormonal.therapy:17:4731:0:137:0.97:1.00:0.11:0.20), (Parapro teinaemia:12:4736:0:137:0.97:1.00:0.08:0.15), (Disease.complication:10:4738:0:137:0.97:1.00:0. 07:0.13), (Shoulder.operation:10:4738:0:137:0.97:1.00:0.07:0.13), (Cardiac.amyloidosis:9:4739:0: 137:0.97:1.00:0.06:0.12), (Hepatic.encephalopathy.prophylaxis:9:4739:0:137:0.97:1.00:0.06:0.1 2), (Mucopolysaccharidosis.IV:9:4739:0:137:0.97:1.00:0.06:0.12), (Feeling.cold:7:4741:0:137:0.9 7:1.00:0.05:0.09), (Vitamin.B12:6:4742:0:137:0.97:1.00:0.04:0.08), (Neuromuscular.blockade:28: 4719:2:136:0.97:0.93:0.17:0.29), (Gestational.hypertension:21:4726:0:138:0.97:1.00:0.13:0.23), (Malignant.neoplasm.of.uterine.adnexa:17:4730:0:138:0.97:1.00:0.11:0.20), (Vomiting.in.pregnan cy:17:4730:0:138:0.97:1.00:0.11:0.20), (Bronchial.obstruction:13:4734:0:138:0.97:1.00:0.09:0.1 6), (Atelectasis:9:4738:0:138:0.97:1.00:0.06:0.12), (Duchenne.muscular.dystrophy:8:4739:0:138: 0.97:1.00:0.05:0.10), (Oesophagogastroduodenoscopy:4:4743:0:138:0.97:1.00:0.03:0.05), (Leuka emia.recurrent:41:4705:0:139:0.97:1.00:0.23:0.37), (Synovitis:32:4714:1:138:0.97:0.97:0.19:0.32), (Precursor.T-lymphoblastic.lymphoma/leukaemia:27:4719:0:139:0.97:1.00:0.16:0.28), (Marginal.zone.lympho ma:21:4725:0:139:0.97:1.00:0.13:0.23), (Purpura:16:4730:0:139:0.97:1.00:0.10:0.19), (Small.fibr e.neuropathy:15:4731:0:139:0.97:1.00:0.10:0.18), (Retinopathy:14:4732:0:139:0.97:1.00:0.09:0.1 7), (Vertigo.positional:12:4734:0:139:0.97:1.00:0.08:0.15), (Epididymitis:11:4735:0:139:0.97:1.0 0:0.07:0.14), (Foot.operation:11:4735:0:139:0.97:1.00:0.07:0.14), (Lipids.decreased:10:4736:0:13 9:0.97:1.00:0.07:0.13), (Promotion.of.wound.healing:10:4736:0:139:0.97:1.00:0.07:0.13), (Allerg y.test:6:4740:0:139:0.97:1.00:0.04:0.08), (Urea.cycle.enzyme.deficiency:6:4740:0:139:0.97:1.00: 0.04:0.08), (Ammonia.abnormal:5:4741:0:139:0.97:1.00:0.03:0.07), (Endoscopy.large.bowel:5:47 41:0:139:0.97:1.00:0.03:0.07), (Alcohol.detoxification:4:4742:0:139:0.97:1.00:0.03:0.05), (Herpe s.ophthalmic:14:4731:0:140:0.97:1.00:0.09:0.17), (Iron.metabolism.disorder:13:4732:0:140:0.97: 1.00:0.08:0.16), (Foot.deformity:11:4734:0:140:0.97:1.00:0.07:0.14), (Skin.erosion:11:4734:0:14 0:0.97:1.00:0.07:0.14), (Diabetic.foot.infection:7:4738:0:140:0.97:1.00:0.05:0.09), (Schizophrenia,.disorganised.type:7:4738:0:140:0.97:1.00:0.05:0.09), (Weight.loss.diet:6:4739:0:140:0.97:1.00: 0.04:0.08), (Tachyphrenia:26:4718:0:141:0.97:1.00:0.16:0.27), (Complications.of.transplanted.kid ney:18:4726:0:141:0.97:1.00:0.11:0.20), (Injection.site.reaction:18:4726:0:141:0.97:1.00:0.11:0.2 0), (Patent.ductus.arteriosus:11:4733:0:141:0.97:1.00:0.07:0.13), (Oesophageal.pain:3:4741:0:141:0.97:1.00:0.02:0.04), (Cerebral.toxoplasmosis:40:4703:3:139:0.97:0.93:0.22:0.36), (Inflammator y.carcinoma.of.the.breast:19:4724:0:142:0.97:1.00:0.12:0.21), (Hypomania:15:4728:0:142:0.97:1 0.00:0.10:0.17), (Rash.pustular:9:4734:0:142:0.97:1.00:0.06:0.1 1), (Vitreous.haemorrhage:8:4735: 0:142:0.97:1.00:0.05:0.10), (Infrequent.bowel.movements:5:4738:0:142:0.97:1.00:0.03:0.07), (Ce llulitis.staphylococcal:4:4739:0:142:0.97:1.00:0.03:0.05), (Skin.cosmetic.procedure:4:4739:0:142: 0.97:1.00:0.03:0.05), (Therapeutic.gargle:20:4722:0:143:0.97:1.00:0.12:0.22), (Von.Willebrand's. disease:17:4725:0:143:0.97:1.00:0.11:0.19), (Genital.neoplasm.malignant.female:14:4728:0:143: 0.97:1.00:0.09:0.16), (Paranasal.sinus.hypersecretion:14:4728:0:143:0.97:1.00:0.09:0.16), (Road.t raffic.accident:14:4728:0:143:0.97:1.00:0.09:0.16), (Thromboangiitis.obliterans:14:4728:0:143:0. 97:1.00:0.09:0.16), (Binge.eating:9:4733:0:143:0.97:1.00:0.06:0.11), (Aspiration:6:4736:0:143:0. 97:1.00:0.04:0.08), (Syphilis:35:4706:1:143:0.97:0.97:0.20:0.33), (Mycobacterium.abscessus.infe ction:18:4723:0:144:0.97:1.00:0.11:0.20), (Thrombotic.thrombocytopenic.purpura:17:4724:0:144: 0.97:1.00:0.11:0.19), (Enteral.nutrition:16:4725:0:144:0.97:1.00:0.10:0.18), (Gastrointestinal.neo plasm:16:4725:0:144:0.97:1.00:0.10:0.18), (Caesarean.section:14:4727:0:144:0.97:1.00:0.09:0.16), (Drug.interaction:13:4728:0:144:0.97:1.00:0.08:0.15), (Muscle.contractions.involuntary:9:4732: 0:144:0.97:1.00:0.06:0.11), (Ankle.fracture:20:4720:0:145:0.97:1.00:0.12:0.22), (Abdominal.absc ess:19:4721:0:145:0.97:1.00:0.12:0.21), (Dermatitis.diaper:16:4724:0:145:0.97:1.00:0.10:0.18), (Metrorrhagia:16:4724:0:145:0.97:1.00:0.10:0.18), (Blood.cortisol.decreased:15:4725:0:145:0.97: 1.00:0.09:0.17), (Blood.magnesium:14:4726:0:145:0.97:1.00:0.09:0.16), (Vitamin.B6.deficiency: 14:4726:1:144:0.97:0.93:0.09:0.16), (Nosocomial.infection:8:4732:0:145:0.97:1.00:0.05:0.10), (N euromyopathy:2:4738:0:145:0.97:1.00:0.01:0.03), (Demyelination:29:4710:0:146:0.97:1.00:0.17: 0.28), (Rheumatoid.vasculitis:24:4715:0:146:0.97:1.00:0.14:0.25), (Fear:21:4718:0:146:0.97:1.00: 0.13:0.22), (Malignant.hypertension:17:4722:0:146:0.97:1.00:0.10:0.19), (Claustrophobia:14:472 5:0:146:0.97:1.00:0.09:0.16), (Orchitis:13:4726:1:145:0.97:0.93:0.08:0.15), (Abdominal.sepsis:5: 4734:0:146:0.97:1.00:0.03:0.06), (Chelation.therapy:25:4713:0:147:0.97:1.00:0.15:0.25), (Arthriti s.enteropathic:21:4717:0:147:0.97:1.00:0.13:0.22), (Cardiac.discomfort:11:4727:0:147:0.97:1.00: 0.07:0.13), (Cystitis.noninfective:11:4727:0:147:0.97:1.00:0.07:0.13), (Injection.site.pain:11:4727:0:147:0.97:1.00:0.07:0.13), (Haemorrhagic.diathesis:7:4731:0:147:0.97:1.00:0.05:0.09), (Synovia 1.sarcoma:20:4717:0:148:0.97:1.00:0.12:0.21), (Gingival.pain:18:4719:0:148:0.97:1.00:0.11:0.20), (Transfusion.reaction:18:4719:0:148:0.97:1.00:0.11:0.20), (Eye.haemorrhage:17:4720:0:148:0.9 7:1.00:0.10:0.19), (Hemiparesis:16:4721:0:148:0.97:1.00:0.10:0.18), (Panic.reaction:13:4724:0:14 8:0.97:1.00:0.08:0.15), (Subdural.haematoma:9:4728:0:148:0.97:1.00:0.06:0.11), (Bruxism:7:473 0:0:148:0.97:1.00:0.05:0.09), (Vulvovaginal.burning.sensation:7:4730:0:148:0.97:1.00:0.05:0.09), (Aplasia:26:4710:0:149:0.97:1.00:0.15:0.26), (Uterine.haemorrhage:24:4712:0:149:0.97:1.00:0.1 4:0.24), (Oesophageal.cancer.metastatic:22:4714:0:149:0.97:1.00:0.13:0.23), (Erythema.multifor me:19:4717:0:149:0.97:1.00:0.11:0.20), (Acute.psychosis:16:4720:0:149:0.97:1.00:0.10:0.18), (V ascular.device.user:15:4721:0:149:0.97:1.00:0.09:0.17), (Acinetobacter.infection:14:4722:0:149: 0.97:1.00:0.09:0.16), (Pathological.fracture:13:4723:0:149:0.97:1.00:0.08:0.15), (Peripheral.coldn ess:13:4723:0:149:0.97:1.00:0.08:0.15), (Blood.oestrogen.abnormal:12:4724:0:149:0.97:1.00:0.0 7:0.14), (Hypertensive.cardiomyopathy:11:4725:0:149:0.97:1.00:0.07:0.13), (Wrong.drug.adminis tered:6:4730:0:149:0.97:1.00:0.04:0.07), (Hyperaesthesia:5:4731:0:149:0.97:1.00:0.03:0.06), (Tak ayasu's.arteritis:35:4700:0:150:0.97:1.00:0.19:0.32), (Progressive.multifocal.leukoencephalopath y:30:4705:0:150:0.97:1.00:0.17:0.29), (Cholangitis.sclerosing:26:4709:0:150:0.97:1.00:0.15:0.26), (Strongyloidiasis:21:4714:0:150:0.97:1.00:0.12:0.22), (Bronchopulmonary.aspergillosis.allergic: 19:4716:1:149:0.97:0.95:0.11:0.20), (Diffuse.large.B-cell.lymphoma.refractory:19:4716:0:150:0.97:1.00:0.11:0.20), (Haemoglobin.abnormal:18:4717: 0:150:0.97:1.00:0.11:0.19), (Spinal.cord.disorder:17:4718:0:150:0.97:1.00:0.10:0.18), (Mass:15:4 720:0:150:0.97:1.00:0.09:0.17), (Cardioversion:12:4723:0:150:0.97:1.00:0.07:0.14), (Castleman's. disease:34:4700:0:151:0.97:1.00:0.18:0.31), (Angiosarcoma:24:4710:0:151:0.97:1.00:0.14:0.24), (Pre-eclampsia:21:4713:0:151:0.97:1.00:0.12:0.22), (Antiandrogen.therapy:17:4717:0:151:0.97:1.00:0 0.10:0.18), (Serum.ferritin.decreased:16:4718:0:151:0.97:1.00:0.10:0.17), (Complication.associated.with.device:14:4720:0:151:0.97:1.00:0.08:0.16), (Infected.skin.ulcer:13:4721:0:151:0.97:1.00:0. 08:0.15), (Proctitis.ulcerative:13:4721:0:151:0.97:1.00:0.08:0.15), (Computerised.tomogram.abdo men:12:4722:0:151:0.97:1.00:0.07:0.14), (Infectious.pleural.effusion:9:4725:0:151:0.97:1.00:0.0 6:0.11), (Neoplasm.progression:26:4707:0:152:0.97:1.00:0.15:0.25), (Coccidioidomycosis:24:470 9:0:152:0.97:1.00:0.14:0.24), (Neuromyelitis.optica.spectrum.disorder:24:4709:0:152:0.97:1.00:0 0.14:0.24), (Sialoadenitis:24:4709:0:152:0.97:1.00:0.14:0.24), (Tubulointerstitial.nephritis:22:4711: 0:152:0.97:1.00:0.13:0.22), (Antiphospholipid.antibodies:19:4714:0:152:0.97:1.00:0.11:0.20), (A drenocortical.carcinoma:18:4715:0:152:0.97:1.00:0.11:0.19), (Eosinophilia:18:4715:0:152:0.97:1 0.00:0.11:0.19), (Failure.to.thrive:15:4718:0:152:0.97:1.00:0.09:0.16), (Haemodilution:15:4718:0:1 52:0.97:1.00:0.09:0.16), (Hyperaldosteronism:14:4719:0:152:0.97:1.00:0.08:0.16), (Endocarditis. bacterial:4:4729:0:152:0.97:1.00:0.03:0.05), (Hepatic.cancer.metastatic:37:4695:0:153:0.97:1.00: 0.19:0.33), (Scleritis:29:4703:0:153:0.97:1.00:0.16:0.27), (Lethargy:20:4712:0:153:0.97:1.00:0.12: 0.21), (Metastases.to.pleura:18:4714:0:153:0.97:1.00:0.11:0.19), (Joint.effusion:17:4715:0:153:0. 97:1.00:0.10:0.18), (Cryopyrin.associated.periodic.syndrome:16:4716:0:153:0.97:1.00:0.09:0.17), (Head.and.neck.cancer.metastatic:9:4723:0:153:0.97:1.00:0.06:0.1 1), (Arteriogram.coronary:6:4 726:0:153:0.97:1.00:0.04:0.07), (Infective.exacerbation.of.chronic.obstructive.airways.disease:6: 4726:0:153:0.97:1.00:0.04:0.07), (Testis.cancer:29:4702:0:154:0.97:1.00:0.16:0.27), (Venoocclusi ve.liver.disease:29:4702:0:154:0.97:1.00:0.16:0.27), (Thrombocytopenic.purpura:19:4712:0:154: 0.97:1.00:0.11:0.20), (Blood.magnesium.abnormal:17:4714:0:154:0.97:1.00:0.10:0.18), (Prematur e.baby:17:4714:0:154:0.97:1.00:0.10:0.18), (Nodule:13:4718:0:154:0.97:1.00:0.08:0.14), (Localis ed.oedema:11:4720:0:154:0.97:1.00:0.07:0.13), (Rhabdomyolysis:7:4724:0:154:0.97:1.00:0.04:0. 08), (Blood.growth.hormone:22:4708:0:155:0.97:1.00:0.12:0.22), (Cytomegalovirus.colitis:22:47 08:0:155:0.97:1.00:0.12:0.22), (Psychotherapy:15:4715:0:155:0.97:1.00:0.09:0.16), (Drug.therapy.enhancement:14:4716:0:155:0.97:1.00:0.08:0.15), (Subcutaneous.abscess:14:4716:0:155:0.97:1. 00:0.08:0.15), (Alcohol.use:13:4717:0:155:0.97:1.00:0.08:0.14), (Lung.abscess:12:4718:0:155:0.9 7:1.00:0.07:0.13), (Gingival.disorder:11:4719:0:155:0.97:1.00:0.07:0.12), (Renal.pain:9:4721:0:1 55:0.97:1.00:0.05:0.10), (Somnambulism:9:4721:0:155:0.97:1.00:0.05:0.10), (Periodontal.disease: 8:4722:0:155:0.97:1.00:0.05:0.09), (Glomerulonephritis.membranoproliferative:29:4700:0:156:0. 97:1.00:0.16:0.27), (Tuberous.sclerosis:29:4700:1:155:0.97:0.97:0.16:0.27), (Jugular.vein.thromb osis:26:4703:0:156:0.97:1.00:0.14:0.25), (Lennox-Gastaut.syndrome:20:4709:0:156:0.97:1.00:0.11:0.20), (Threatened.labour:20:4709:0:156:0.97:1. 00:0.11:0.20), (Agoraphobia:16:4713:0:156:0.97:1.00:0.09:0.17), (Skin.discolouration:15:4714:0: 156:0.97:1.00:0.09:0.16), (Duodenogastric.reflux:11:4718:0:156:0.97:1.00:0.07:0.12), (Anaemia. of.malignant.disease:9:4720:0:156:0.97:1.00:0.05:0.10), (Social.anxiety.disorder:9:4720:0:156:0. 97:1.00:0.05:0.10), (Myoclonic.epilepsy:23:4705:0:157:0.97:1.00:0.13:0.23), (Impaired.healing:2 1:4707:0:157:0.97:1.00:0.12:0.21), (Cryoglobulinaemia:19:4709:0:157:0.97:1.00:0.11:0.19), (Pre cancerous.skin.lesion:15:4713:0:157:0.97:1.00:0.09:0.16), (Dyschezia:14:4714:0:157:0.97:1.00:0 0.08:0.15), (Polyp:14:4714:0:157:0.97:1.00:0.08:0.15), (Dry.age-related.macular.degeneration:13:4715:0:157:0.97:1.00:0.08:0.14), (Breast.disorder:12:4716:0:157:0.97:1.00:0.07:0.13), (Urinary.tract.obstruction:12:4716:0:157:0.97:1.00:0.07:0.13), (Epicondylit is:11:4717:0:157:0.97:1.00:0.07:0.12), (Metastatic.pain:11:4717:1:156:0.97:0.92:0.07:0.12), (Ath erosclerosis.prophylaxis:9:4719:0:157:0.97:1.00:0.05:0.10), (Senile.dementia:9:4719:0:157:0.97: 1.00:0.05:0.10), (Abscess.limb:7:4721:0:157:0.97:1.00:0.04:0.08), (Aortic.dissection:6:4722:0:15 7:0.97:1.00:0.04:0.07), (Radiation.skin.injury:4:4724:0:157:0.97:1.00:0.02:0.05), (Pulse.abnormal: 2:4726:0:157:0.97:1.00:0.01:0.02), (Post.transplant.lymphoproliferative.disorder:47:4680:0:158: 0.97:1.00:0.23:0.37), (Invasive.breast.carcinoma:21:4706:0:158:0.97:1.00:0.12:0.21), (Factor.V.d eficiency:19:4708:0:158:0.97:1.00:0.11:0.19), (Aortic.valve.incompetence:13:4714:0:158:0.97:1. 00:0.08:0.14), (Atrial.thrombosis:11:4716:0:158:0.97:1.00:0.07:0.12), (Facial.neuralgia:10:4717:0: 158:0.97:1.00:0.06:0.11), (Testicular.failure:9:4718:0:158:0.97:1.00:0.05:0.10), (Low.density.lip oprotein:7:4720:0:158:0.97:1.00:0.04:0.08), (Cytomegalovirus.test.positive:43:4683:1:158:0.97:0 0.98:0.21:0.35), (Blood.insulin:29:4697:0:159:0.97:1.00:0.15:0.27), (Precocious.puberty:21:4705:0: 159:0.97:1.00:0.12:0.21), (Mycotic.allergy:17:4709:0:159:0.97:1.00:0.10:0.18), (Alopecia.areata: 16:4710:0:159:0.97:1.00:0.09:0.17), (Secondary.adrenocortical.insufficiency:15:4711:0:159:0.97: 1.00:0.09:0.16), (Antithrombin.III.deficiency:13:4713:0:159:0.97:1.00:0.08:0.14), (Cryptococcosi s:37:4688:0:160:0.97:1.00:0.19:0.32), (Diffuse.large.B-cell.lymphoma.stage.IV:25:4700:0:160:0.97:1.00:0.14:0.24), (Drug.ineffective:24:4701:0:160:0.9 7:1.00:0.13:0.23), (Ganglioglioma:22:4703:1:159:0.97:0.96:0.12:0.22), (Biliary.cirrhosis:21:4704: 0:160:0.97:1.00:0.12:0.21), (Impulsive.behaviour:17:4708:0:160:0.97:1.00:0.10:0.18), (Postpartu m.depression:17:4708:0:160:0.97:1.00:0.10:0.18), (Heart.valve.incompetence:15:4710:0:160:0.9 7:1.00:0.09:0.16), (Endometrial.adenocarcinoma:14:4711:0:160:0.97:1.00:0.08:0.15), (Menopaus al.disorder:8:4717:0:160:0.97:1.00:0.05:0.09), (Anuria:7:4718:0:160:0.97:1.00:0.04:0.08), (Gastri c.lavage:7:4718:0:160:0.97:1.00:0.04:0.08), (Systemic.mycosis:30:4694:0:161:0.97:1.00:0.16:0.2 7), (Congenital.generalised.lipodystrophy:15:4709:0:161:0.97:1.00:0.09:0.16), (Malignant.glioma: 14:4710:0:161:0.97:1.00:0.08:0.15), (Quadriplegia:14:4710:0:161:0.97:1.00:0.08:0.15), (Liposarc oma:12:4712:0:161:0.97:1.00:0.07:0.13), (Overgrowth.bacterial:9:4715:0:161:0.97:1.00:0.05:0.1 0), (Renal.colic:8:4716:0:161:0.97:1.00:0.05:0.09), (Hormone.suppression.therapy:19:4704:0:162: 0.97:1.00:0.10:0.19), (Tonsil.cancer:13:4710:0:162:0.97:1.00:0.07:0.14), (Emergency.care:12:471 1:0:162:0.97:1.00:0.07:0.13), (Squamous.cell.carcinoma.of.the.tongue:12:4711:0:162:0.97:1.00:0 0.07:0.13), (Peripheral.ischaemia:10:4713:0:162:0.97:1.00:0.06:0.11), (Systemic.mastocytosis:10:4 713:0:162:0.97:1.00:0.06:0.1 1), (Eosinophilic.oesophagitis:9:4714:0:162:0.97:1.00:0.05:0.10), (C ystoid.macular.oedema:38:4684:0:163:0.97:1.00:0.19:0.32), (Sensory.disturbance:21:4701:0:163: 0.97:1.00:0.11:0.20), (Therapeutic.embolisation:17:4705:0:163:0.97:1.00:0.09:0.17), (Mucopolys accharidosis.I:14:4708:0:163:0.97:1.00:0.08:0.15), (Upper.respiratory.tract.congestion:14:4708:0: 163:0.97:1.00:0.08:0.15), (Ehlers-Danlos.syndrome:11:4711:0:163:0.97:1.00:0.06:0.12), (Enema.administration:8:4714:0:163:0.97: 1.00:0.05:0.09), (Vulvovaginal.discomfort:6:4716:0:163:0.97:1.00:0.04:0.07), (Oropharyngeal.ca ndidiasis:4:4718:0:163:0.97:1.00:0.02:0.05), (Temporal.lobe.epilepsy:26:4695:0:164:0.97:1.00:0. 14:0.24), (Peroneal.nerve.palsy:19:4702:0:164:0.97:1.00:0.10:0.19), (Dupuytren's.contracture:14: 4707:0:164:0.97:1.00:0.08:0.15), (Mental.status.changes:14:4707:0:164:0.97:1.00:0.08:0.15), (De myelinating.polyneuropathy:13:4708:0:164:0.97:1.00:0.07:0.14), (Biopsy:10:4711:0:164:0.97:1.0 0:0.06:0.11), (Regurgitation:10:4711:0:164:0.97:1.00:0.06:0.11), (Vitreous.adhesions:10:4711:0:1 64:0.97:1.00:0.06:0.11), (Graft.versus.host.disease.in.skin:48:4672:0:165:0.97:1.00:0.23:0.37), (In traocular.lens.implant:28:4692:1:164:0.97:0.97:0.15:0.25), (Retroviral.infection:24:4696:0:165:0. 97:1.00:0.13:0.23), (Viraemia:18:4702:0:165:0.97:1.00:0.10:0.18), (Injection.site.erythema:15:47 05:0:165:0.97:1.00:0.08:0.15), (Mitral.valve.repair:15:4705:0:165:0.97:1.00:0.08:0.15), (Pneumo nia.pseudomonal:13:4707:0:165:0.97:1.00:0.07:0.14), (Cardiac.ablation:12:4708:0:165:0.97:1.00: 0.07:0.13), (Nocardiosis:38:4681:0:166:0.97:1.00:0.19:0.31), (Chronic.granulomatous.disease:29: 4690:0:166:0.97:1.00:0.15:0.26), (Retroperitoneal.cancer:23:4696:0:166:0.97:1.00:0.12:0.22), (Sp inal.cord.compression:20:4699:0:166:0.97:1.00:0.11:0.19), (Drug-induced.liver.injury:17:4702:0:166:0.97:1.00:0.09:0.17), (Nasal.inflammation:13:4706:0:166:0.9 7:1.00:0.07:0.14), (Thrombosis.in.device:13:4706:0:166:0.97:1.00:0.07:0.14), (Seborrhoea:5:4714: 0:166:0.97:1.00:0.03:0.06), (Homocystinuria:4:4715:0:166:0.97:1.00:0.02:0.05), (Ewing's.sarco ma:34:4684:0:167:0.97:1.00:0.17:0.29), (Vehicle.solution.use:23:4695:0:167:0.97:1.00:0.12:0.22), (Anal.fistula:19:4699:0:167:0.97:1.00:0.10:0.19), (Blood.insulin.abnormal:16:4702:0:167:0.97: 1.00:0.09:0.16), (Escherichia.bacteraemia:16:4702:0:167:0.97:1.00:0.09:0.16), (Laryngeal.cancer: 12:4706:0:167:0.97:1.00:0.07:0.13), (Empyema:7:4711:0:167:0.97:1.00:0.04:0.08), (Resuscitation: 6:4712:0:167:0.97:1.00:0.03:0.07), (Metastases.to.spine:37:4680:0:168:0.97:1.00:0.18:0.31), (Ret inal.oedema:33:4684:1:167:0.97:0.97:0.17:0.28), (Collagen.disorder:32:4685:0:168:0.97:1.00:0.1 6:0.28), (Diffuse.large.B-cell.lymphoma.recurrent:29:4688:0:168:0.97:1.00:0.15:0.26), (Rash.macular:16:4701:0:168:0.97: 1.00:0.09:0.16), (Gastric.cancer.stage.IV:15:4702:0:168:0.97:1.00:0.08:0.15), (Hallucination,.visu al:13:4704:0:168:0.97:1.00:0.07:0.13), (Meniscus.injury:12:4705:0:168:0.97:1.00:0.07:0.13), (Su barachnoid.haemorrhage:8:4709:0:168:0.97:1.00:0.05:0.09), (Amenorrhoea:26:4690:0:169:0.97:1 0.00:0.13:0.24), (Presyncope:21:4695:0:169:0.97:1.00:0.11:0.20), (Pneumonia.klebsiella:20:4696:0: 169:0.97:1.00:0.11:0.19), (Gigantism:17:4699:0:169:0.97:1.00:0.09:0.17), (Restrictive.pulmonar y.disease:17:4699:0:169:0.97:1.00:0.09:0.17), (Adenovirus.infection:15:4701:0:169:0.97:1.00:0.0 8:0.15), (Mental.impairment:15:4701:0:169:0.97:1.00:0.08:0.15), (Central.pain.syndrome:14:4702:0:169:0.97:1.00:0.08:0.14), (Mucopolysaccharidosis:11:4705:0:169:0.97:1.00:0.06:0.12), (Trache al.cancer:11:4705:0:169:0.97:1.00:0.06:0.12), (Mast.cell.activation.syndrome:10:4706:0:169:0.97:1.00:0.06:0.11), (Endodontic.procedure:8:4708:0:169:0.97:1.00:0.05:0.09), (Bacterial.disease.car rier:22:4693:0:170:0.97:1.00:0.11:0.21), (Blood.phosphorus.abnormal:18:4697:0:170:0.97:1.00:0 0.10:0.17), (Pancreatic.enzymes:18:4697:0:170:0.97:1.00:0.10:0.17), (Sciatic.nerve.injury:11:4704: 0:170:0.97:1.00:0.06:0.11), (Procedural.hypotension:9:4706:0:170:0.97:1.00:0.05:0.10), (Aplasia. pure.red.cell:30:4684:0:171:0.96:1.00:0.15:0.26), (Selective.IgA.immunodeficiency:18:4696:0:17 1:0.96:1.00:0.10:0.17), (Stress.echocardiogram:17:4697:0:171:0.96:1.00:0.09:0.17), (Facial.paraly sis:15:4699:0:171:0.96:1.00:0.08:0.15), (Muscle.contracture:14:4700:0:171:0.96:1.00:0.08:0.14), (Myelopathy:14:4700:0:171:0.96:1.00:0.08:0.14), (Arteriovenous.malformation:12:4702:0:171:0. 96:1.00:0.07:0.12), (Complement.deficiency.disease:11:4703:0:171:0.96:1.00:0.06:0.11), (Multipl e.system.atrophy:7:4707:0:171:0.96:1.00:0.04:0.08), (Malignant.neoplasm.of.unknown.primary.s ite:27:4686:0:172:0.96:1.00:0.14:0.24), (Hypolipidaemia:21:4692:0:172:0.96:1.00:0.11:0.20), (Pa rtial.seizures.with.secondary.generalisation:20:4693:0:172:0.96:1.00:0.10:0.19), (Premature.men opause:20:4693:0:172:0.96:1.00:0.10:0.19), (Cytokine.release.syndrome:18:4695:0:172:0.96:1.00:0.09:0.17), (Oestrogen.deficiency:17:4696:0:172:0.96:1.00:0.09:0.17), (Stenotrophomonas.infecti on:16:4697:0:172:0.96:1.00:0.09:0.16), (Anorectal.disorder:13:4700:0:172:0.96:1.00:0.07:0.13), (Blood.phosphorus:12:4701:0:172:0.96:1.00:0.07:0.12), (Dumping.syndrome:11:4702:0:172:0.96: 1.00:0.06:0.11), (Ear.discomfort:11:4702:0:172:0.96:1.00:0.06:0.1 1), (Cholecystitis.acute:9:4704: 0:172:0.96:1.00:0.05:0.09), (Lower.respiratory.tract.congestion:8:4705:0:172:0.96:1.00:0.04:0.09), (Liver.injury:22:4690:0:173:0.96:1.00:0.11:0.20), (Nephritis:22:4690:0:173:0.96:1.00:0.11:0.20), (Gallbladder.disorder:17:4695:1:172:0.96:0.94:0.09:0.16), (Ophthalmological.examination:16:4 696:0:173:0.96:1.00:0.08:0.16), (Colitis.ischaemic:13:4699:0:173:0.96:1.00:0.07:0.13), (Paranasal.sinus.discomfort:13:4699:0:173:0.96:1.00:0.07:0.13), (Oral.discomfort:10:4702:0:173:0.96:1.00: 0.05:0.10), (Retinal.disorder:7:4705:0:173:0.96:1.00:0.04:0.07), (Thalassaemia.beta:31:4680:0:17 4:0.96:1.00:0.15:0.26), (Breast.cancer.stage.III:24:4687:0:174:0.96:1.00:0.12:0.22), (Gastrointesti nal.mucosal.disorder:20:4691:0:174:0.96:1.00:0.10:0.19), (Intracardiac.thrombus:18:4693:0:174: 0.96:1.00:0.09:0.17), (Impetigo:15:4696:0:174:0.96:1.00:0.08:0.15), (Ophthalmic.herpes.zoster:1 4:4697:0:174:0.96:1.00:0.07:0.14), (Pleural.mesothelioma:12:4699:0:174:0.96:1.00:0.06:0.12), (F ace.oedema:10:4701:0:174:0.96:1.00:0.05:0.10), (Malignant.connective.tissue.neoplasm:10:4701: 0:174:0.96:1.00:0.05:0.10), (Infertility:32:4678:1:174:0.96:0.97:0.16:0.27), (Adverse.reaction:25: 4685:0:175:0.96:1.00:0.13:0.22), (Corneal.transplant:21:4689:0:175:0.96:1.00:0.11:0.19), (Fibrosi s:19:4691:0:175:0.96:1.00:0.10:0.18), (Infection.parasitic:19:4691:0:175:0.96:1.00:0.10:0.18), (Fa milial.risk.factor:17:4693:0:175:0.96:1.00:0.09:0.16), (Animal.bite:15:4695:0:175:0.96:1.00:0.08: 0.15), (Arteriospasm.coronary:14:4696:0:175:0.96:1.00:0.07:0.14), (Emotional.distress:14:4696: 0:175:0.96:1.00:0.07:0.14), (Blepharospasm:11:4699:0:175:0.96:1.00:0.06:0.11), (Onychoclasis:1 1:4699:0:175:0.96:1.00:0.06:0.11), (Nasopharyngeal.cancer:30:4679:0:176:0.96:1.00:0.15:0.25), (Intraoperative.care:27:4682:2:174:0.96:0.93:0.13:0.23), (Acquired.haemophilia:25:4684:1:175:0. 96:0.96:0.13:0.22), (Arthritis.reactive:25:4684:0:176:0.96:1.00:0.12:0.22), (Haemolysis:22:4687: 0:176:0.96:1.00:0.11:0.20), (Duodenitis:21:4688:0:176:0.96:1.00:0.11:0.19), (Cheilitis:17:4692:0: 176:0.96:1.00:0.09:0.16), (Euphoric.mood:17:4692:0:176:0.96:1.00:0.09:0.16), (Intracranial.aneur ysm:15:4694:0:176:0.96:1.00:0.08:0.15), (Feeding.disorder:14:4695:1:175:0.96:0.93:0.07:0.14), (Premature.delivery:14:4695:0:176:0.96:1.00:0.07:0.14), (Bronchoscopy:12:4697:1:175:0.96:0.92: 0.06:0.12), (Somatic.symptom.disorder:8:4701:0:176:0.96:1.00:0.04:0.08), (Fistula:17:4691:0:17 7:0.96:1.00:0.09:0.16), (Asteatosis:16:4692:0:177:0.96:1.00:0.08:0.15), (Systemic.inflammatory.r esponse.syndrome:16:4692:0:177:0.96:1.00:0.08:0.15), (Bronchopneumopathy:12:4696:0:177:0. 96:1.00:0.06:0.12), (Thinking.abnormal:12:4696:0:177:0.96:1.00:0.06:0.12), (Chondrocalcinosis. pyrophosphate:28:4679:0:178:0.96:1.00:0.14:0.24), (Anaemia.of.chronic.disease:24:4683:0:178: 0.96:1.00:0.12:0.21), (Pain.of.skin:20:4687:0:178:0.96:1.00:0.10:0.18), (Blood.thyroid.stimulatin g.hormone.increased:16:4691:0:178:0.96:1.00:0.08:0.15), (Nephrosclerosis:9:4698:0:178:0.96:1. 00:0.05:0.09), (Skin.candida:4:4703:0:178:0.96:1.00:0.02:0.04), (Hypervolaemia:1:4706:0:178:0. 96:1.00:0.01:0.01), (Optic.neuritis:31:4675:0:179:0.96:1.00:0.15:0.26), (Costochondritis:25:4681: 0:179:0.96:1.00:0.12:0.22), (Transaminases.increased:25:4681:0:179:0.96:1.00:0.12:0.22), (Odyn ophagia:20:4686:0:179:0.96:1.00:0.10:0.18), (Haemosiderosis:17:4689:0:179:0.96:1.00:0.09:0.16), (Hairy.cell.leukaemia:17:4689:0:179:0.96:1.00:0.09:0.16), (Intentional.product.misuse:10:4696: 0:179:0.96:1.00:0.05:0.10), (Glossitis:8:4698:0:179:0.96:1.00:0.04:0.08), (Mitral.valve.disease:46:4659:0:180:0.96:1.00:0.20:0.34), (Stenosis:19:4686:0:180:0.96:1.00:0.10:0.17), (Neuroendocrine.carcinoma.of.the.skin:18:4687:0:180:0.96:1.00:0.09:0.17), (Complement.factor.abnormal:16:468 9:0:180:0.96:1.00:0.08:0.15), (Non-cardiac.chest.pain:10:4695:0:180:0.96:1.00:0.05:0.10), (T-cell.type.acute.leukaemia:59:4645:1:180:0.96:0.98:0.25:0.39), (Intraductal.proliferative.breast.les ion:34:4670:0:181:0.96:1.00:0.16:0.27), (Diabetic.eye.disease:25:4679:1:180:0.96:0.96:0.12:0.22), (Dysthymic.disorder:21:4683:0:181:0.96:1.00:0.10:0.19), (Blood.calcium.increased:15:4689:0:1 81:0.96:1.00:0.08:0.14), (Bladder.discomfort:12:4692:0:181:0.96:1.00:0.06:0.12), (Adjustment.di sorder:23:4680:0:182:0.96:1.00:0.11:0.20), (Lung.neoplasm:22:4681:0:182:0.96:1.00:0.11:0.19), (Stevens-Johnson.syndrome:20:4683:0:182:0.96:1.00:0.10:0.18), (Drooling:10:4693:0:182:0.96:1.00:0.05: 0.10), (Apathy:8:4695:0:182:0.96:1.00:0.04:0.08), (Haemodynamic.instability:38:4664:4:179:0.96: 0.90:0.18:0.29), (Autism:32:4670:0:183:0.96:1.00:0.15:0.26), (Extranodal.marginal.zone.B-cell.lymphoma.(MALT.type):32:4670:0:183:0.96:1.00:0.15:0.26), (Brain.abscess:21:4681:0:183: 0.96:1.00:0.10:0.19), (Squamous.cell.carcinoma.of.skin:18:4684:0:183:0.96:1.00:0.09:0.16), (Refl ux.laryngitis:15:4687:0:183:0.96:1.00:0.08:0.14), (Haematocrit.decreased:13:4689:0:183:0.96:1.0 0:0.07:0.12), (Oedema.due.to.renal.disease:12:4690:0:183:0.96:1.00:0.06:0.12), (IgA.nephropathy: 37:4664:0:184:0.96:1.00:0.17:0.29), (Invasive.lobular.breast.carcinoma:24:4677:0:184:0.96:1.00:0.12:0.21), (Radiotherapy:24:4677:0:184:0.96:1.00:0.12:0.21), (Salivary.gland.cancer:24:4677:0: 184:0.96:1.00:0.12:0.21), (Breast.cancer.male:21:4680:0:184:0.96:1.00:0.10:0.19), (Pigmentation. disorder:15:4686:0:184:0.96:1.00:0.08:0.14), (Sleep.terror:15:4686:0:184:0.96:1.00:0.08:0.14), (C holecystectomy:14:4687:0:184:0.96:1.00:0.07:0.13), (Head.discomfort:13:4688:0:184:0.96:1.00: 0.07:0.12), (Azotaemia:12:4689:0:184:0.96:1.00:0.06:0.12), (Kaposi's.sarcoma:67:4633:0:185:0.9 6:1.00:0.27:0.42), (Hypoproteinaemia:25:4675:0:185:0.96:1.00:0.12:0.21), (Skin.reaction:25:4675: 0:185:0.96:1.00:0.12:0.21), (Coronary.artery.insufficiency:24:4676:1:184:0.96:0.96:0.12:0.21), (Myeloproliferative.disorder:22:4678:0:185:0.96:1.00:0.11:0.19), (Small.cell.lung.cancer.extensiv e.stage:15:4685:0:185:0.96:1.00:0.08:0.14), (Status.asthmaticus:15:4685:0:185:0.96:1.00:0.08:0. 14), (POEMS.syndrome:14:4686:0:185:0.96:1.00:0.07:0.13), (Spinal.fusion.surgery:13:4687:0:18 5:0.96:1.00:0.07:0.12), (Bone.giant.cell.tumour:4:4696:0:185:0.96:1.00:0.02:0.04), (Acute.promy elocytic.leukaemia:35:4664:1:185:0.96:0.97:0.16:0.27), (Metastatic.carcinoid.tumour:29:4670:0: 186:0.96:1.00:0.13:0.24), (Venous.occlusion:17:4682:0:186:0.96:1.00:0.08:0.15), (Nasal.deconge stion.therapy:16:4683:0:186:0.96:1.00:0.08:0.15), (Muscle.rigidity:13:4686:0:186:0.96:1.00:0.07: 0.12), (Mobility.decreased:35:4663:0:187:0.96:1.00:0.16:0.27), (Anaplastic.astrocytoma:26:4672: 1:186:0.96:0.96:0.12:0.22), (Venous.thrombosis.limb:21:4677:0:187:0.96:1.00:0.10:0.18), (Ovari an.cancer.stage.III:20:4678:0:187:0.96:1.00:0.10:0.18), (Lip.and/or.oral.cavity.cancer:16:4682:0: 187:0.96:1.00:0.08:0.15), (Abnormal.dreams:14:4684:0:187:0.96:1.00:0.07:0.13), (Lymphangiolei omyomatosis:13:4685:0:187:0.96:1.00:0.07:0.12), (High.density.lipoprotein.decreased:75:4622:6: 182:0.96:0.93:0.29:0.44), (Organ.transplant:32:4665:0:188:0.96:1.00:0.15:0.25), (Polychondritis: 29:4668:0:188:0.96:1.00:0.13:0.24), (Joint.stiffness:27:4670:0:188:0.96:1.00:0.13:0.22), (Red.blo od.cell.abnormality:24:4673:0:188:0.96:1.00:0.11:0.20), (Hypocholesterolaemia:17:4680:0:188:0 0.96:1.00:0.08:0.15), (Sacroiliitis:16:4681:0:188:0.96:1.00:0.08:0.15), (Prostatic.specific.antigen:1 5:4682:0:188:0.96:1.00:0.07:0.14), (Eye.discharge:13:4684:0:188:0.96:1.00:0.06:0.12), (Laryngiti s:13:4684:0:188:0.96:1.00:0.06:0.12), (Enzyme.abnormality:12:4685:0:188:0.96:1.00:0.06:0.11), (Carotid.arteriosclerosis:9:4688:0:188:0.96:1.00:0.05:0.09), (Vitamin.E.deficiency:9:4688:0:188: 0.96:1.00:0.05:0.09), (Wound.complication:6:4691:0:188:0.96:1.00:0.03:0.06), (Gene.mutation:5 0:4646:1:188:0.96:0.98:0.21:0.35), (Induction.and.maintenance.of.anaesthesia:39:4657:1:188:0.9 6:0.98:0.17:0.29), (Growth.retardation:31:4665:0:189:0.96:1.00:0.14:0.25), (Lymphadenitis:31:46 65:0:189:0.96:1.00:0.14:0.25), (Neurodermatitis:27:4669:0:189:0.96:1.00:0.13:0.22), (Oropharyn geal.cancer:17:4679:0:189:0.96:1.00:0.08:0.15), (Promotion.of.peripheral.circulation:14:4682:0:1 89:0.96:1.00:0.07:0.13), (Acid.base.balance.abnormal:13:4683:0:189:0.96:1.00:0.06:0.12), (Bladd er.transitional.cell.carcinoma.metastatic:9:4687:0:189:0.96:1.00:0.05:0.09), (Toxoplasmosis:64:4 631:1:189:0.96:0.98:0.25:0.40), (Metastases.to.meninges:38:4657:0:190:0.96:1.00:0.17:0.29), (Op hthalmic.herpes.simplex:27:4668:1:189:0.96:0.96:0.13:0.22), (Pleuritic.pain:27:4668:1:189:0.96: 0.96:0.13:0.22), (Refractory.cytopenia.with.unilineage.dysplasia:24:4671:0:190:0.96:1.00:0.11:0. 20), (Osteogenesis.imperfecta:23:4672:0:190:0.96:1.00:0.11:0.19), (Petit.mal.epilepsy:22:4673:0: 190:0.96:1.00:0.10:0.19), (Body.tinea:18:4677:0:190:0.96:1.00:0.09:0.16), (Dental.caries:13:4682: 0:190:0.96:1.00:0.06:0.12), (Drug.level.decreased:11:4684:0:190:0.96:1.00:0.05:0.10), (CREST.s yndrome:56:4638:2:189:0.96:0.97:0.23:0.37), (Cor.pulmonale:37:4657:0:191:0.96:1.00:0.16:0.28), (Meningioma:27:4667:1:190:0.96:0.96:0.12:0.22), (Deafness:17:4677:0:191:0.96:1.00:0.08:0.1 5), (Delusional.disorder,.unspecified.type:12:4682:0:191:0.96:1.00:0.06:0.11), (Irrigation.therapy: 10:4684:1:190:0.96:0.91:0.05:0.09), (Thyroid.cancer.metastatic:7:4687:0:191:0.96:1.00:0.04:0.07), (Cardiac.flutter:49:4644:0:192:0.96:1.00:0.20:0.34), (Rectal.adenocarcinoma:28:4665:0:192:0.9 6:1.00:0.13:0.23), (Sinus.node.dysfunction:28:4665:0:192:0.96:1.00:0.13:0.23), (Adjustment.disor der.with.depressed.mood:23:4670:0:192:0.96:1.00:0.11:0.19), (Oxygen.supplementation:17:4676:0:192:0.96:1.00:0.08:0.15), (Blood.albumin.decreased:13:4680:0:192:0.96:1.00:0.06:0.12), (Tour ette's.disorder:66:4626:2:191:0.96:0.97:0.26:0.41), (Meningitis.cryptococcal:52:4640:3:190:0.96: 0.95:0.21:0.35), (Cytopenia:34:4658:1:192:0.96:0.97:0.15:0.26), (Microscopic.polyangiitis:31:46 61:0:193:0.96:1.00:0.14:0.24), (Diastolic.dysfunction:22:4670:1:192:0.96:0.96:0.10:0.19), (Anore ctal.discomfort:20:4672:0:193:0.96:1.00:0.09:0.17), (Vena.cava.thrombosis:20:4672:0:193:0.96:1 0.00:0.09:0.17), (Hip.surgery:17:4675:0:193:0.96:1.00:0.08:0.15), (Increased.bronchial.secretion:1 6:4676:0:193:0.96:1.00:0.08:0.14), (Asthma-chronic.obstructive.pulmonary.disease.overlap.syndrome:13:4679:0:193:0.96:1.00:0.06:0.12), (G lioma:50:4641:1:193:0.96:0.98:0.21:0.34), (Craniocerebral.injury:36:4655:1:193:0.96:0.97:0.16:0 0.27), (Gastrointestinal.inflammation:31:4660:0:194:0.96:1.00:0.14:0.24), (Epidural.anaesthesia:30: 4661:4:190:0.96:0.88:0.14:0.24), (Hypnotherapy:19:4672:0:194:0.96:1.00:0.09:0.16), (Colon.can cer.stage.IV:17:4674:0:194:0.96:1.00:0.08:0.15), (Blood.oestrogen:16:4675:0:194:0.96:1.00:0.08: 0.14), (Non-alcoholic.steatohepatitis:13:4678:0:194:0.96:1.00:0.06:0.12), (Electrocardiogram.QT.prolonged:7: 4684:0:194:0.96:1.00:0.03:0.07), (Full.blood.count.decreased:23:4667:0:195:0.96:1.00:0.11:0.19), (Automatic.bladder:16:4674:0:195:0.96:1.00:0.08:0.14), (Adult.T-cell.lymphoma/leukaemia:38:4651:1:195:0.96:0.97:0.16:0.28), (Blindness:30:4659:0:196:0.96:1. 00:0.13:0.23), (Febrile.bone.marrow.aplasia:28:4661:0:196:0.96:1.00:0.13:0.22), (Glomeruloneph ritis.membranous:27:4662:0:196:0.96:1.00:0.12:0.22), (Appendix.cancer:18:4671:0:196:0.96:1.0 0:0.08:0.16), (Blood.pressure.inadequately.controlled:18:4671:0:196:0.96:1.00:0.08:0.16), (Migra ine.with.aura:18:4671:0:196:0.96:1.00:0.08:0.16), (Hypovolaemia:9:4680:0:196:0.96:1.00:0.04:0. 08), (Axial.spondyloarthritis:32:4656:0:197:0.96:1.00:0.14:0.25), (Anaemia.vitamin.B12.deficien cy:29:4659:0:197:0.96:1.00:0.13:0.23), (Respiration.abnormal:27:4661:0:197:0.96:1.00:0.12:0.22), (Periodic.limb.movement.disorder:25:4663:0:197:0.96:1.00:0.11:0.20), (Haematoma:21:4667:0: 197:0.96:1.00:0.10:0.18), (Lichen.sclerosus:15:4673:0:197:0.96:1.00:0.07:0.13), (Mucopolysacch aridosis.VI:15:4673:0:197:0.96:1.00:0.07:0.13), (Urine.alkalinisation.therapy:15:4673:0:197:0.96:1.00:0.07:0.13), (Mucopolysaccharidosis.II:14:4674:0:197:0.96:1.00:0.07:0.12), (B-cell.type.acute.leukaemia:49:4638:3:195:0.96:0.94:0.20:0.33), (Anal.cancer:30:4657:0:198:0.96: 1.00:0.13:0.23), (Premature.labour:25:4662:0:198:0.96:1.00:0.11:0.20), (Bladder.irritation:12:467 5:0:198:0.96:1.00:0.06:0.11), (Graft.versus.host.disease.in.gastrointestinal.tract:47:4639:0:199:0. 96:1.00:0.19:0.32), (Atrial.septal.defect:44:4642:0:199:0.96:1.00:0.18:0.31), (Glomerulonephritis: 31:4655:0:199:0.96:1.00:0.13:0.24), (Breast.cancer.stage.II:28:4658:0:199:0.96:1.00:0.12:0.22), (Asthma.exercise.induced:26:4660:0:199:0.96:1.00:0.12:0.21), (Chronic.lymphocytic.leukaemia.r efractory:23:4663:0:199:0.96:1.00:0.10:0.19), (Immunoglobulins.decreased:23:4663:0:199:0.96:1 0.00:0.10:0.19), (Cardiac.assistance.device.user:20:4666:0:199:0.96:1.00:0.09:0.17), (Myopathy:51: 4634:0:200:0.96:1.00:0.20:0.34), (Liver.transplant.rejection:31:4654:0:200:0.96:1.00:0.13:0.24), (Exostosis:30:4655:1:199:0.96:0.97:0.13:0.23), (Nephroprotective.therapy:30:4655:0:200:0.96:1. 00:0.13:0.23), (Hepatitis.C.virus.test:28:4657:0:200:0.96:1.00:0.12:0.22), (Dyspnoea.exertional:2 3:4662:0:200:0.96:1.00:0.10:0.19), (Cerebral.ischaemia:15:4670:0:200:0.96:1.00:0.07:0.13), (Cor onary.angioplasty:11:4674:0:200:0.96:1.00:0.05:0.10), (Blood.oestrogen.decreased:26:4658:0:20 1:0.96:1.00:0.11:0.21), (Gastric.pH.increased:26:4658:0:201:0.96:1.00:0.11:0.21), (Erythema.nod osum:22:4662:0:201:0.96:1.00:0.10:0.18), (Substance.use:21:4663:0:201:0.96:1.00:0.09:0.17), (A naplastic.thyroid.cancer:15:4669:0:201:0.96:1.00:0.07:0.13), (Gastroenteritis.viral:13:4671:0:201: 0.96:1.00:0.06:0.11), (Thrombophlebitis.superficial:12:4672:0:201:0.96:1.00:0.06:0.1 1), (Depend ence:7:4677:0:201:0.96:1.00:0.03:0.07), (Ulcer.haemorrhage:68:4615:4:198:0.96:0.94:0.26:0.40), (Venoocclusive.disease:40:4643:0:202:0.96:1.00:0.17:0.28), (Arterial.thrombosis:31:4652:0:202: 0.96:1.00:0.13:0.23), (Anaplastic.large.cell.lymphoma.T-.and.null-cell.types:25:4658:0:202:0.96:1.00:0.11:0.20), (Enteritis:25:4658:0:202:0.96:1.00:0.11:0.20), (Re nal.neoplasm:25:4658:0:202:0.96:1.00:0.11:0.20), (Lung.infiltration:21:4662:0:202:0.96:1.00:0.0 9:0.17), (Glomerulonephritis.chronic:20:4663:0:202:0.96:1.00:0.09:0.17), (Blood.iron.abnormal:1 2:4671:0:202:0.96:1.00:0.06:0.11), (Small.intestinal.obstruction:18:4664:0:203:0.96:1.00:0.08:0. 15), (Carcinoid.tumour.of.the.small.bowel:17:4665:0:203:0.96:1.00:0.08:0.14), (Remission.not.ac hieved:16:4666:0:203:0.96:1.00:0.07:0.14), (Myofascial.pain.syndrome:14:4668:0:203:0.96:1.00: 0.06:0.12), (Endoscopy:13:4669:0:203:0.96:1.00:0.06:0.11), (Angioplasty:92:4589:2:202:0.96:0.9 8:0.31:0.47), (Immunomodulatory.therapy:57:4624:0:204:0.96:1.00:0.22:0.36), (Schizoaffective.d isorder.bipolar.type:49:4632:2:202:0.96:0.96:0.20:0.32), (Epstein-Barr.virus.infection:46:4635:0:204:0.96:1.00:0.18:0.31), (Malignant.lymphoid.neoplasm:43:4638:0:204:0.96:1.00:0.17:0.30), (Cardiac.valve.prosthesis.user:32:4649:0:204:0.96:1.00:0.14:0.24), (F ocal.segmental.glomerulosclerosis:30:4651:0:204:0.96:1.00:0.13:0.23), (EGFR.gene.mutation:13: 4668:0:204:0.96:1.00:0.06:0.11), (Eye.operation:13:4668:0:204:0.96:1.00:0.06:0.11), (Blood.coun t.abnormal:47:4633:0:205:0.96:1.00:0.19:0.31), (Fungaemia:27:4653:0:205:0.96:1.00:0.12:0.21), (Migraine.without.aura:24:4656:0:205:0.96:1.00:0.10:0.19), (Mastocytosis:15:4665:0:205:0.96:1. 00:0.07:0.13), (Bone.marrow.disorder:28:4651:0:206:0.96:1.00:0.12:0.21), (Acid.base.balance:26: 4653:0:206:0.96:1.00:0.11:0.20), (Microcytic.anaemia:22:4657:0:206:0.96:1.00:0.10:0.18), (Vulv ovaginal.pain:14:4665:0:206:0.96:1.00:0.06:0.12), (Incision.site.pain:11:4668:0:206:0.96:1.00:0. 05:0.10), (Urine.output:9:4670:0:206:0.96:1.00:0.04:0.08), (Meningitis.bacterial:38:4640:0:207:0. 96:1.00:0.16:0.27), (Haematochezia:26:4652:0:207:0.96:1.00:0.11:0.20), (Osteonecrosis.of.jaw:25: 4653:0:207:0.96:1.00:0.11:0.19), (Occipital.neuralgia:12:4666:0:207:0.96:1.00:0.05:0.10), (Appe ndicitis:11:4667:0:207:0.96:1.00:0.05:0.10), (Endoscopy.upper.gastrointestinal.tract:7:4671:0:20 7:0.96:1.00:0.03:0.06), (Focal.dyscognitive.seizures:28:4649:0:208:0.96:1.00:0.12:0.21), (Pseudo membranous.colitis:26:4651:0:208:0.96:1.00:0.11:0.20), (Hypogonadism.male:18:4659:0:208:0.9 6:1.00:0.08:0.15), (Niemann-Pick.disease:15:4662:0:208:0.96:1.00:0.07:0.13), (Ejection.fraction.decreased:12:4665:0:208:0.9 6:1.00:0.05:0.10), (Carotid.artery.occlusion:18:4658:0:209:0.96:1.00:0.08:0.15), (Gastrointestinal. hypomotility:16:4660:0:209:0.96:1.00:0.07:0.13), (Diabetic.ulcer:145:4530:13:197:0.96:0.92:0.4 2:0.58), (Heart.rate.decreased:126:4549:7:203:0.96:0.95:0.38:0.55), (Neck.injury:28:4647:0:210:0 0.96:1.00:0.12:0.21), (Brain.neoplasm.benign:27:4648:1:209:0.96:0.96:0.11:0.20), (Gallbladder.ca ncer:21:4654:1:209:0.96:0.95:0.09:0.17), (Skin.papilloma:20:4655:0:210:0.96:1.00:0.09:0.16), (A naphylactic.shock:16:4659:0:210:0.96:1.00:0.07:0.13), (Catheter.placement:12:4663:0:210:0.96: 1.00:0.05:0.10), (Phantom.pain:92:4582:3:208:0.96:0.97:0.31:0.47), (Thyroid.hormones.decrease d:67:4607:2:209:0.96:0.97:0.24:0.39), (Bacterial.vaginosis:30:4644:0:211:0.96:1.00:0.12:0.22), (Tobacco.abuse:17:4657:0:211:0.96:1.00:0.07:0.14), (Ileus.paralytic:16:4658:0:211:0.96:1.00:0.0 7:0.13), (Lacunar.infarction:15:4659:0:211:0.96:1.00:0.07:0.12), (Hordeolum:13:4661:0:211:0.96: 1.00:0.06:0.11), (Exposure.during.pregnancy:73:4600:0:212:0.96:1.00:0.26:0.41), (Local.swelling: 51:4622:1:211:0.96:0.98:0.19:0.32), (Lupus-like.syndrome:38:4635:1:211:0.96:0.97:0.15:0.26), (Urine.flow.decreased:33:4640:0:212:0.96:1. 00:0.13:0.24), (Tongue.neoplasm.malignant.stage.unspecified:25:4648:0:212:0.96:1.00:0.11:0.19), (Swelling.face:24:4649:0:212:0.96:1.00:0.10:0.18), (Adrenal.gland.cancer:20:4653:0:212:0.96: 1.00:0.09:0.16), (Bronchiolitis:20:4653:0:212:0.96:1.00:0.09:0.16), (Gastrointestinal.bacterial.ove rgrowth:18:4655:0:212:0.96:1.00:0.08:0.15), (Femur.fracture:17:4656:0:212:0.96:1.00:0.07:0.14), (Pyelonephritis.acute:16:4657:0:212:0.96:1.00:0.07:0.13), (Malignant.neoplasm.of.renal.pelvis:1 4:4659:0:212:0.96:1.00:0.06:0.12), (Vitamin.K.deficiency:13:4660:0:212:0.96:1.00:0.06:0.11), (Wound.infection.staphylococcal:12:4661:0:212:0.96:1.00:0.05:0.10), (Autism.spectrum.disorder: 45:4627:1:212:0.96:0.98:0.18:0.30), (Aneurysm:39:4633:0:213:0.96:1.00:0.15:0.27), (Body.temp erature:21:4651:0:213:0.96:1.00:0.09:0.16), (Neoplasm.prostate:17:4655:0:213:0.96:1.00:0.07:0. 14), (Hypertensive.crisis:16:4656:0:213:0.96:1.00:0.07:0.13), (Hypopharyngeal.cancer:15:4657:0: 213:0.96:1.00:0.07:0.12), (Meningitis.fungal:6:4666:0:213:0.96:1.00:0.03:0.05), (Mucormycosis: 54:4617:0:214:0.96:1.00:0.20:0.34), (Metastases.to.bone.marrow:50:4621:1:213:0.96:0.98:0.19:0 0.32), (Bone.neoplasm:44:4627:1:213:0.96:0.98:0.17:0.29), (Drug.intolerance:31:4640:0:214:0.96: 1.00:0.13:0.22), (Carotid.artery.disease:25:4646:0:214:0.96:1.00:0.10:0.19), (Liver.abscess:25:46 46:0:214:0.96:1.00:0.10:0.19), (Tic:24:4647:0:214:0.96:1.00:0.10:0.18), (Respiratory.syncytial.vir us.infection:22:4649:0:214:0.96:1.00:0.09:0.17), (Burkitt's.lymphoma:41:4629:0:215:0.96:1.00:0. 16:0.28), (Organising.pneumonia:34:4636:1:214:0.96:0.97:0.14:0.24), (Splenomegaly:29:4641:0: 215:0.96:1.00:0.12:0.21), (Thyroid.neoplasm:29:4641:0:215:0.96:1.00:0.12:0.21), (Chronic.lymp hocytic.leukaemia.recurrent:27:4643:0:215:0.96:1.00:0.11:0.20), (White.blood.cell.disorder:25:4 645:0:215:0.96:1.00:0.10:0.19), (Infective.pulmonary.exacerbation.of.cystic.fibrosis:17:4653:1:2 14:0.96:0.94:0.07:0.14), (Intraocular.pressure.test:16:4654:0:215:0.96:1.00:0.07:0.13), (Rapid.eye.movements.sleep.abnormal:12:4658:0:215:0.96:1.00:0.05:0.10), (Pneumocystis.jirovecii.infectio n:53:4616:1:215:0.96:0.98:0.20:0.33), (Chondropathy:32:4637:0:216:0.96:1.00:0.13:0.23), (Cryin g:31:4638:0:216:0.96:1.00:0.13:0.22), (Phenylketonuria:30:4639:0:216:0.96:1.00:0.12:0.22), (An gioimmunoblastic.T-cell.lymphoma:29:4640:0:216:0.96:1.00:0.12:0.21), (Bacterial.sepsis:27:4642:0:216:0.96:1.00:0. 11:0.20), (Osteitis.deformans:27:4642:0:216:0.96:1.00:0.11:0.20), (Blood.immunoglobulin.G.decr eased:22:4647:0:216:0.96:1.00:0.09:0.17), (Intraocular.pressure.test.abnormal:21:4648:0:216:0.9 6:1.00:0.09:0.16), (Eye.swelling:19:4650:0:216:0.96:1.00:0.08:0.15), (Vascular.graft:113:4555:6: 211:0.96:0.95:0.35:0.51), (Cardiac.murmur:87:4581:3:214:0.96:0.97:0.29:0.45), (Seasonal.affecti ve.disorder:44:4624:0:217:0.96:1.00:0.17:0.29), (Cervical.spinal.stenosis:21:4647:0:217:0.96:1.0 0:0.09:0.16), (Vitamin.B1.deficiency:20:4648:0:217:0.96:1.00:0.08:0.16), (Mitral.valve.replacem ent:54:4613:2:216:0.96:0.96:0.20:0.33), (Arteritis:29:4638:0:218:0.96:1.00:0.12:0.21), (Gastric.m ucosal.lesion:17:4650:0:218:0.96:1.00:0.07:0.13), (Gastric.haemorrhage:29:4637:0:219:0.96:1.00: 0.12:0.21), (Atypical.pneumonia:25:4641:0:219:0.96:1.00:0.10:0.19), (Nerve.block:24:4642:5:21 4:0.96:0.83:0.10:0.18), (Ileus:22:4644:0:219:0.96:1.00:0.09:0.17), (Embolic.stroke:19:4647:0:219: 0.96:1.00:0.08:0.15), (Hypoglobulinaemia:48:4617:0:220:0.95:1.00:0.18:0.30), (Atrioventricular. block:40:4625:0:220:0.95:1.00:0.15:0.27), (Factor.IX.deficiency:27:4638:1:219:0.95:0.96:0.11:0. 20), (Chorea:26:4639:0:220:0.95:1.00:0.11:0.19), (Hypophosphatasia:21:4644:0:220:0.95:1.00:0. 09:0.16), (Non-24-hour.sleep-wake.disorder:21:4644:0:220:0.95:1.00:0.09:0.16), (Acute.pulmonary.oedema:17:4648:0:220:0.9 5:1.00:0.07:0.13), (Nail.infection:17:4648:0:220:0.95:1.00:0.07:0.13), (Vascular.dementia:14:465 1:0:220:0.95:1.00:0.06:0.11), (Hypernatraemia:9:4656:0:220:0.95:1.00:0.04:0.08), (Dyspareunia: 125:4539:8:213:0.95:0.94:0.37:0.53), (Metabolic.syndrome:51:4613:0:221:0.95:1.00:0.19:0.32), (Hepatic.neoplasm:39:4625:0:221:0.95:1.00:0.15:0.26), (Spondylolisthesis:24:4640:0:221:0.95:1. 00:0.10:0.18), (Increased.upper.airway.secretion:22:4642:0:221:0.95:1.00:0.09:0.17), (Autonomic.failure.syndrome:20:4644:0:221:0.95:1.00:0.08:0.15), (Lipidosis:182:4481:15:207:0.95:0.92:0.4 7:0.62), (Musculoskeletal.disorder:61:4602:3:219:0.95:0.95:0.22:0.35), (Systemic.scleroderma:54: 4609:1:221:0.95:0.98:0.20:0.33), (Haemophilia:43:4620:0:222:0.95:1.00:0.16:0.28), (Colony.sti mulating.factor.therapy:34:4629:1:221:0.95:0.97:0.13:0.23), (Seronegative.arthritis:32:4631:0:22 2:0.95:1.00:0.13:0.22), (Choroidal.neovascularisation:29:4634:2:220:0.95:0.94:0.12:0.21), (Kerati tis:29:4634:0:222:0.95:1.00:0.12:0.21), (Mydriasis:22:4641:1:221:0.95:0.96:0.09:0.17), (Bone.can cer.metastatic:21:4642:0:222:0.95:1.00:0.09:0.16), (Scar:21:4642:0:222:0.95:1.00:0.09:0.16), (Hi p.fracture:19:4644:0:222:0.95:1.00:0.08:0.15), (Lower.urinary.tract.symptoms:15:4648:0:222:0.9 5:1.00:0.06:0.12), (Catheter.site.pain:10:4653:0:222:0.95:1.00:0.04:0.08), (Senile.osteoporosis:77: 4585:2:221:0.95:0.97:0.26:0.41), (Acute.leukaemia:60:4602:4:219:0.95:0.94:0.22:0.35), (Scan.my ocardial.perfusion:53:4609:0:223:0.95:1.00:0.19:0.32), (Spinal.anaesthesia:34:4628:3:220:0.95:0. 92:0.13:0.23), (Drug.withdrawal.maintenance.therapy:31:4631:0:223:0.95:1.00:0.12:0.22), (Ataxi a:26:4636:0:223:0.95:1.00:0.10:0.19), (Cardiac.pacemaker.insertion:99:4562:9:215:0.95:0.92:0.3 2:0.47), (Haematopoietic.stem.cell.mobilisation:44:4617:0:224:0.95:1.00:0.16:0.28), (Labour.indu ction:35:4626:0:224:0.95:1.00:0.14:0.24), (Blood.pressure.systolic:30:4631:0:224:0.95:1.00:0.12: 0.21), (Ovarian.epithelial.cancer:29:4632:0:224:0.95:1.00:0.11:0.21), (Splenectomy:26:4635:0:22 4:0.95:1.00:0.10:0.19), (Thyroid.mass:25:4636:0:224:0.95:1.00:0.10:0.18), (Metastases.to.periton eum:45:4615:1:224:0.95:0.98:0.17:0.29), (Pulmonary.sarcoidosis:38:4622:1:224:0.95:0.97:0.15:0 0.25), (Agranulocytosis:26:4634:0:225:0.95:1.00:0.10:0.19), (Cardiomegaly:26:4633:0:226:0.95:1. 00:0.10:0.19), (Allergy.to.arthropod.sting:24:4635:0:226:0.95:1.00:0.10:0.18), (Device.occlusion: 24:4635:0:226:0.95:1.00:0.10:0.18), (Dandruff:23:4636:0:226:0.95:1.00:0.09:0.17), (Ductal.adeno carcinoma.of.pancreas:17:4642:0:226:0.95:1.00:0.07:0.13), (Left.ventricular.failure:61:4597:2:22 5:0.95:0.97:0.21:0.35), (Coeliac.disease:36:4622:1:226:0.95:0.97:0.14:0.24), (Still's.disease.adult. onset:36:4622:0:227:0.95:1.00:0.14:0.24), (Platelet.aggregation:31:4627:0:227:0.95:1.00:0.12:0.2 1), (Eructation:24:4634:0:227:0.95:1.00:0.10:0.17), (Ammonia.increased:18:4640:0:227:0.95:1.00: 0.07:0.14), (Aortic.stenosis:49:4608:0:228:0.95:1.00:0.18:0.30), (Therapy.naive:47:4610:3:225:0. 95:0.94:0.17:0.29), (Still's.disease:43:4614:0:228:0.95:1.00:0.16:0.27), (Vitamin.C.deficiency:28: 4629:0:228:0.95:1.00:0.11:0.20), (Thyroid.operation:23:4634:0:228:0.95:1.00:0.09:0.17), (Allergi c.sinusitis:109:4547:7:222:0.95:0.94:0.33:0.49), (Intermittent.claudication:90:4566:3:226:0.95:0. 97:0.28:0.44), (Plantar.fasciitis:45:4611:1:228:0.95:0.98:0.16:0.28), (Pancreas.transplant:29:4627: 0:229:0.95:1.00:0.11:0.20), (Neutropenic.sepsis:27:4629:0:229:0.95:1.00:0.11:0.19), (Mesothelio ma:25:4631:0:229:0.95:1.00:0.10:0.18), (Urinary.tract.infection.bacterial:19:4637:0:229:0.95:1.0 0:0.08:0.14), (Abdominal.rigidity:17:4639:0:229:0.95:1.00:0.07:0.13), (Systemic.sclerosis:74:458 1:3:227:0.95:0.96:0.25:0.39), (Coronary.artery.occlusion:71:4584:5:225:0.95:0.93:0.24:0.38), (M aternal.therapy.to.enhance.foetal.lung.maturity:57:4598:0:230:0.95:1.00:0.20:0.33), (Vein.disord er:31:4624:0:230:0.95:1.00:0.12:0.21), (Post.procedural.complication:28:4627:0:230:0.95:1.00:0. 11:0.20), (Depressed.level.of.consciousness:23:4632:0:230:0.95:1.00:0.09:0.17), (Cerebral.haemo rrhage:21:4634:0:230:0.95:1.00:0.08:0.15), (Intracranial.pressure.increased:39:4615:0:231:0.95:1 0.00:0.14:0.25), (Nuclear.magnetic.resonance.imaging.brain:28:4626:0:231:0.95:1.00:0.11:0.20), (Pleural.mesothelioma.malignant:13:4641:0:231:0.95:1.00:0.05:0.10), (Osteolysis:31:4622:0:232: 0.95:1.00:0.12:0.21), (Bladder.dysfunction:24:4629:0:232:0.95:1.00:0.09:0.17), (General.sympto m:24:4629:0:232:0.95:1.00:0.09:0.17), (Tumour.associated.fever:16:4637:0:232:0.95:1.00:0.06:0 0.12), (Libido.decreased:65:4587:2:231:0.95:0.97:0.22:0.36), (Vitamin.D.abnormal:48:4604:0:233: 0.95:1.00:0.17:0.29), (Frequent.bowel.movements:33:4619:0:233:0.95:1.00:0.12:0.22), (Enteritis.i nfectious:23:4629:0:233:0.95:1.00:0.09:0.16), (Prophylaxis.against.solar.radiation:22:4630:0:233: 0.95:1.00:0.09:0.16), (Gastric.ulcer.haemorrhage:18:4634:0:233:0.95:1.00:0.07:0.13), (Interverte bral.discitis:16:4636:0:233:0.95:1.00:0.06:0.12), (Pulmonary.thrombosis:176:4475:19:215:0.95:0 0.90:0.45:060), (Pulmonary.mass:46:4605:0:234:0.95:1.00:0.16:0.28), (Enuresis:41:4610:0:234:0.9 5:1.00:0.15:0.26), (Neuritis:38:4613:0:234:0.95:1.00:0.14:0.25), (Withdrawal.syndrome:33:4618: 0:234:0.95:1.00:0.12:0.22), (Blood.testosterone:25:4626:0:234:0.95:1.00:0.10:0.18), (Eczema.aste atotic:20:4631:0:234:0.95:1.00:0.08:0.15), (Post.laminectomy.syndrome:122:4528:10:225:0.95:0. 92:0.35:0.51), (Neurological.symptom:33:4617:0:235:0.95:1.00:0.12:0.22), (Sinus.operation:32:4 618:1:234:0.95:0.97:0.12:0.21), (Enterobacter.infection:22:4628:0:235:0.95:1.00:0.09:0.16), (Gro wth.of.eyelashes:66:4583:1:235:0.95:0.99:0.22:0.36), (Knee.operation:43:4606:1:235:0.95:0.98:0 0.15:0.27), (Cervical.radiculopathy:35:4614:0:236:0.95:1.00:0.13:0.23), (Alcohol.abuse:24:4625:0: 236:0.95:1.00:0.09:0.17), (Arthritis.infective:23:4626:0:236:0.95:1.00:0.09:0.16), (Vulvovaginal. pruritus:12:4637:0:236:0.95:1.00:0.05:0.09), (Nerve.compression:196:4452:25:212:0.95:0.89:0.4 8:0.62), (Cardiac.stress.test:87:4561:4:233:0.95:0.96:0.27:0.42), (Eye.allergy:61:4587:3:234:0.95: 0.95:0.21:0.34), (Coronary.artery.stenosis:24:4624:0:237:0.95:1.00:0.09:0.17), (Cardiac.operation: 160:4487:12:226:0.95:0.93:0.41:0.57), (Device.therapy:33:4614:0:238:0.95:1.00:0.12:0.22), (Cys tinosis:24:4623:0:238:0.95:1.00:0.09:0.17), (Oedema.due.to.hepatic.disease:9:4638:0:238:0.95:1. 00:0.04:0.07), (Factor.V.Leiden.mutation:140:4506:16:223:0.95:0.90:0.39:0.54), (Tobacco.user:1 37:4509:16:223:0.95:0.90:0.38:0.53), (Peyronie's.disease:87:4559:7:232:0.95:0.93:0.27:0.42), (Na sal.polyps:34:4612:0:239:0.95:1.00:0.12:0.22), (Malaria.prophylaxis:29:4617:0:239:0.95:1.00:0.1 1:0.20), (Cardiac.fibrillation:26:4620:0:239:0.95:1.00:0.10:0.18), (Spinal.operation:80:4565:2:238: 0.95:0.98:0.25:0.40), (Acne.cystic:64:4581:2:238:0.95:0.97:0.21:0.35), (Acute.respiratory.distres s.syndrome:46:4599:0:240:0.95:1.00:0.16:0.28), (Breast.neoplasm:35:4610:0:240:0.95:1.00:0.13: 0.23), (Hyper.IgM.syndrome:32:4613:1:239:0.95:0.97:0.12:0.21), (Pancreatic.neuroendocrine.tum our.metastatic:28:4617:0:240:0.95:1.00:0.10:0.19), (Thrombolysis:21:4624:0:240:0.95:1.00:0.08: 0.15), (Heparin-induced.thrombocytopenia:12:4633:0:240:0.95:1.00:0.05:0.09), (Tinea.cruris:11:4634:0:240:0.95: 1.00:0.04:0.08), (Vasospasm:11:4634:0:240:0.95:1.00:0.04:0.08), (Lipids:147:4497:15:226:0.95: 0.91:0.39:0.55), (Plasmacytoma:84:4560:1:240:0.95:0.99:0.26:0.41), (Aortic.aneurysm:66:4578:2: 239:0.95:0.97:0.22:0.35), (Oestrogen.therapy:61:4583:3:238:0.95:0.95:0.20:0.34), (Plasma.cell.le ukaemia:47:4597:0:241:0.95:1.00:0.16:0.28), (Plasma.cell.myeloma.in.remission:46:4598:2:239: 0.95:0.96:0.16:0.28), (Anaplastic.large-cell.lymphoma:34:4610:0:241:0.95:1.00:0.12:0.22), (Diabetes.prophylaxis:29:4615:0:241:0.95:1. 00:0.11:0.19), (Investigation:27:4617:1:240:0.95:0.96:0.10:0.18), (Periodontitis:23:4621:0:241:0. 95:1.00:0.09:0.16), (Sputum.abnormal:13:4631:0:241:0.95:1.00:0.05:0.10), (Blood.iron:148:4495: 17:225:0.95:0.90:0.40:0.55), (Ovarian.cyst:75:4568:1:241:0.95:0.99:0.24:0.38), (Ovarian.cancer. metastatic:41:4602:0:242:0.95:1.00:0.14:0.25), (Bile.duct.cancer:24:4619:0:242:0.95:1.00:0.09:0. 17), (Fallopian.tube.cancer:20:4623:0:242:0.95:1.00:0.08:0.14), (Escherichia.urinary.tract.infectio n:13:4630:0:242:0.95:1.00:0.05:0.10), (Muscle.strain:57:4585:1:242:0.95:0.98:0.19:0.32), (Proctit is:21:4621:0:243:0.95:1.00:0.08:0.15), (Glycosylated.haemoglobin.increased:120:4521:7:237:0.9 5:0.94:0.34:0.50), (Premenstrual.dysphoric.disorder:111:4530:7:237:0.95:0.94:0.32:0.48), (Malig nant.neoplasm.progression:85:4556:5:239:0.95:0.94:0.26:0.41), (Blood.glucose.fluctuation:64:45 77:2:242:0.95:0.97:0.21:0.34), (Combined.immunodeficiency:55:4586:0:244:0.95:1.00:0.18:0.31), (Parathyroid.disorder:36:4605:0:244:0.95:1.00:0.13:0.23), (Chronic.respiratory.failure:34:4607: 2:242:0.95:0.94:0.12:0.22), (Alcohol.withdrawal.syndrome:14:4627:0:244:0.95:1.00:0.05:0.10), (Perennial.allergy:6:4635:0:244:0.95:1.00:0.02:0.05), (Lumbar.radiculopathy:137:4503:11:234:0. 95:0.93:0.37:0.53), (Post.coital.contraception:92:4548:6:239:0.95:0.94:0.28:0.43), (Monoclonal.g ammopathy:75:4565:4:241:0.95:0.95:0.24:0.38), (Allergy.to.animal:53:4587:1:244:0.95:0.98:0.1 8:0.30), (Thalassaemia:35:4605:0:245:0.95:1.00:0.13:0.22), (Head.injury:30:4610:0:245:0.95:1.00: 0.11:0.20), (Gastrooesophageal.cancer:21:4619:0:245:0.95:1.00:0.08:0.15), (Peritonitis.bacterial: 20:4620:0:245:0.95:1.00:0.08:0.14), (Oliguria:13:4627:0:245:0.95:1.00:0.05:0.10), (Colitis.micros copic:114:4525:3:243:0.95:0.97:0.32:0.48), (Mitral.valve.incompetence:100:4539:2:244:0.95:0.9 8:0.29:0.45), (B.precursor.type.acute.leukaemia:59:4580:3:243:0.95:0.95:0.20:0.32), (Gastrointest inal.carcinoma:45:4594:1:245:0.95:0.98:0.16:0.27), (Portopulmonary.hypertension:44:4595:0:24 6:0.95:1.00:0.15:0.26), (Detoxification:29:4610:0:246:0.95:1.00:0.11:0.19), (Gouty.arthritis:137:4 501:11:236:0.95:0.93:0.37:0.53), (Thyroid.function.test.abnormal:120:4518:9:238:0.95:0.93:0.34: 0.49), (Hypothalamo-pituitary.disorder:89:4549:1:246:0.95:0.99:0.27:0.42), (Malaria:40:4598:0:247:0.95:1.00:0.14:0.2 4), (Blood.testosterone.abnormal:35:4603:0:247:0.95:1.00:0.12:0.22), (Body.height.below.normal: 34:4604:0:247:0.95:1.00:0.12:0.22), (Androgenetic.alopecia:25:4613:0:247:0.95:1.00:0.09:0.17), (Intentional.overdose:25:4613:1:246:0.95:0.96:0.09:0.17), (Right.ventricular.failure:54:4583:1:24 7:0.95:0.98:0.18:0.30), (Renal.lithiasis.prophylaxis:53:4584:0:248:0.95:1.00:0.18:0.30), (Leiomyo sarcoma:33:4604:0:248:0.95:1.00:0.12:0.21), (Anger:122:4514:11:238:0.95:0.92:0.34:0.49), (Fall: 41:4595:0:249:0.95:1.00:0.14:0.25), (Endocrine.disorder:24:4612:0:249:0.95:1.00:0.09:0.16), (Ce rvicobrachial.syndrome:22:4614:0:249:0.95:1.00:0.08:0.15), (Haematemesis:21:4615:0:249:0.95: 1.00:0.08:0.14), (Breast.pain:19:4617:0:249:0.95:1.00:0.07:0.13), (Laceration:18:4618:0:249:0.95: 1.00:0.07:0.13), (Iritis:49:4586:1:249:0.95:0.98:0.16:0.28), (Phytotherapy:42:4593:0:250:0.95:1. 00:0.14:0.25), (Stress.ulcer:17:4618:0:250:0.95:1.00:0.06:0.12), (Thyroiditis:50:4584:2:249:0.95: 0.96:0.17:0.28), (Astrocytoma,.low.grade:38:4596:1:250:0.95:0.97:0.13:0.23), (Vaginal.disorder:3 4:4600:0:251:0.95:1.00:0.12:0.21), (Emotional.disorder:98:4535:5:247:0.95:0.95:0.28:0.44), (Im mune.tolerance.induction:54:4579:0:252:0.95:1.00:0.18:0.30), (Cutaneous.lupus.erythematosus:4 4:4589:1:251:0.95:0.98:0.15:0.26), (Erosive.oesophagitis:63:4569:1:252:0.95:0.98:0.20:0.33), (Cy tomegalovirus.chorioretinitis:60:4572:1:252:0.95:0.98:0.19:0.32), (Blood.thyroid.stimulating.hor mone.decreased:41:4591:0:253:0.95:1.00:0.14:0.24), (Akathisia:29:4603:0:253:0.95:1.00:0.10:0. 19), (Stress.urinary.incontinence:29:4603:0:253:0.95:1.00:0.10:0.19), (Hyperthermia:24:4608:0:2 53:0.95:1.00:0.09:0.16), (Left.ventricular.dysfunction:90:4541:3:251:0.95:0.97:0.26:0.41), (Thro mbotic.microangiopathy:62:4569:0:254:0.95:1.00:0.20:0.33), (Congenital.hypogammaglobulinae mia:57:4574:1:253:0.95:0.98:0.18:0.31), (Renal.cell.carcinoma.stage.IV:51:4580:1:253:0.95:0.98: 0.17:0.29), (Ventricular.arrhythmia:51:4580:0:254:0.95:1.00:0.17:0.29), (Amyotrophic.lateral.scl erosis:17:4614:0:254:0.95:1.00:0.06:0.12), (Imaging.procedure:15:4616:1:253:0.95:0.94:0.06:0.1 1), (Vaginal.haemorrhage:62:4568:1:254:0.95:0.98:0.20:0.33), (Rectal.cancer.metastatic:31:4599: 0:255:0.95:1.00:0.11:0.20), (Rectal.haemorrhage:28:4602:0:255:0.95:1.00:0.10:0.18), (Ventricula r.fibrillation:25:4605:0:255:0.95:1.00:0.09:0.16), (Urine.abnormality:24:4606:0:255:0.95:1.00:0. 09:0.16), (Scoliosis:192:4437:26:230:0.95:0.88:0.45:0.60), (Hypokalaemic.syndrome:52:4577:1:2 55:0.95:0.98:0.17:0.29), (Hypophagia:24:4605:0:256:0.95:1.00:0.09:0.16), (Refractory.anaemia.w ith.an.excess.of.blasts:22:4607:0:256:0.95:1.00:0.08:0.15), (Malignant.melanoma.stage.IV:21:46 08:0:256:0.95:1.00:0.08:0.14), (Spondyloarthropathy:110:4518:8:249:0.95:0.93:0.31:0.46), (Gesta tional.diabetes:58:4570:0:257:0.95:1.00:0.18:0.31), (Anti-neutrophil.cytoplasmic.antibody.positive.vasculitis:41:4587:0:257:0.95:1.00:0.14:0.24), (Infantile.spasms:28:4600:0:257:0.95:1.00:0.10:0.18), (Arrhythmia.supraventricular:27:4601:0:257:0.95:1. 00:0.10:0.17), (Asthenopia:18:4610:0:257:0.95:1.00:0.07:0.12), (Echocardiogram:130:4497:13:24 5:0.95:0.91:0.35:0.50), (Lipodystrophy.acquired:73:4554:1:257:0.95:0.99:0.22:0.36), (Dental.oper ation:32:4595:0:258:0.95:1.00:0.11:0.20), (Oxygen.saturation.decreased:31:4596:0:258:0.95:1.00: 0.11:0.19), (Motion.sickness:118:4508:10:249:0.95:0.92:0.32:0.48), (Angiogram:64:4562:0:259: 0.95:1.00:0.20:0.33), (Labyrinthitis:40:4586:0:259:0.95:1.00:0.13:0.24), (Sarcoma.metastatic:24:4 602:0:259:0.95:1.00:0.08:0.16), (Spinal.fracture:113:4512:8:252:0.95:0.93:0.31:0.47), (Disease.re currence:86:4539:2:258:0.95:0.98:0.25:0.40), (Central.nervous.system.lymphoma:60:4565:0:260: 0.95:1.00:0.19:0.32), (Hepatitis.C.virus.test.positive:59:4566:0:260:0.95:1.00:0.18:0.31), (Energy. increased:45:4580:1:259:0.95:0.98:0.15:0.26), (Bronchopneumonia:21:4604:1:259:0.95:0.95:0.0 8:0.14), (Mucous.membrane.disorder:18:4607:0:260:0.95:1.00:0.06:0.12), (Skin.abrasion:16:4609: 0:260:0.95:1.00:0.06:0.11), (Haemolytic.anaemia:58:4566:0:261:0.95:1.00:0.18:0.31), (Mycobact erial.infection:48:4576:0:261:0.95:1.00:0.16:0.27), (Blood.calcium.abnormal:38:4586:1:260:0.95: 0.97:0.13:0.23), (Skin.fissures:20:4604:0:261:0.95:1.00:0.07:0.13), (Androgen.deficiency:173:44 50:20:242:0.95:0.90:0.42:0.57), (Temporomandibular.joint.syndrome:141:4482:12:250:0.95:0.92:0.36:0.52), (Feeling.abnormal:93:4530:4:258:0.95:0.96:0.26:0.42), (Prostatism:36:4587:0:262:0. 95:1.00:0.12:0.22), (Neuroblastoma:35:4588:0:262:0.95:1.00:0.12:0.21), (Catheterisation.cardiac: 28:4595:0:262:0.95:1.00:0.10:0.18), (Dysphonia:23:4600:0:262:0.95:1.00:0.08:0.15), (Dermatoph ytosis.of.nail:11:4612:0:262:0.95:1.00:0.04:0.08), (Menstruation.irregular:126:4496:7:256:0.95:0 0.95:0.33:0.49), (Cluster.headache:76:4546:5:258:0.95:0.94:0.23:0.37), (Postmenopause:64:4558:1: 262:0.95:0.98:0.20:0.33), (Helicobacter.gastritis:29:4593:0:263:0.95:1.00:0.10:0.18), (Nightmare: 188:4433:22:242:0.95:0.90:0.44:0.59), (Haemochromatosis:176:4445:16:248:0.95:0.92:0.42:0.5 7), (Pancreatic.disorder:168:4453:18:246:0.95:0.90:0.41:0.56), (Prophylaxis.of.neural.tube.defect: 65:4556:1:263:0.95:0.98:0.20:0.33), (Atypical.mycobacterial.infection:61:4560:0:264:0.95:1.00:0 0.19:0.32), (Blood.pressure.systolic.increased:57:4564:2:262:0.95:0.97:0.18:0.30), (Neoplasm.prop hylaxis:47:4574:0:264:0.95:1.00:0.15:0.26), (Cardiac.failure.acute:45:4576:0:264:0.95:1.00:0.15: 0.25), (Lichen.planus:40:4581:0:264:0.95:1.00:0.13:0.23), (Eating.disorder:28:4593:0:264:0.95:1. 00:0.10:0.18), (Prinzmetal.angina:26:4595:0:264:0.95:1.00:0.09:0.16), (Supraventricular.extrasyst oles:26:4595:0:264:0.95:1.00:0.09:0.16), (Acarodermatitis:23:4598:0:264:0.95:1.00:0.08:0.15), (Heart.valve.replacement:159:4461:15:250:0.95:0.91:0.39:0.55), (Hypercoagulation:98:4522:6:25 9:0.95:0.94:0.27:0.43), (Pleurisy:39:4581:0:265:0.95:1.00:0.13:0.23), (Platelet.count.increased:34: 4586:0:265:0.95:1.00:0.11:0.20), (Pneumonia.staphylococcal:20:4600:0:265:0.95:1.00:0.07:0.13), (Thyroid.therapy:173:4446:19:247:0.95:0.90:0.41:0.57), (Blood.phosphorus.increased:72:4547: 3:263:0.95:0.96:0.21:0.35), (Spinal.cord.injury:199:4419:24:243:0.95:0.89:0.45:0.60), (Oesophag eal.disorder:149:4469:13:254:0.95:0.92:0.37:0.53), (Back.injury:108:4510:10:257:0.95:0.92:0.30: 0.45), (Pituitary.tumour:62:4556:0:267:0.95:1.00:0.19:0.32), (Hyperparathyroidism.primary:20:4 598:0:267:0.95:1.00:0.07:0.13), (Primary.immunodeficiency.syndrome:171:4446:24:244:0.95:0.8 8:0.41:0.56), (Sneezing:103:4514:6:262:0.95:0.94:0.28:0.43), (Hallucination,.auditory:78:4539:4: 264:0.95:0.95:0.23:0.37), (Cardiac.arrest:73:4544:4:264:0.95:0.95:0.22:0.35), (Overweight:63:45 54:1:267:0.95:0.98:0.19:0.32), (Acute.myeloid.leukaemia.recurrent:43:4574:1:267:0.95:0.98:0.14: 0.24), (Oestrogen.receptor.positive.breast.cancer:39:4578:1:267:0.95:0.98:0.13:0.23), (Intentional.self-injury:33:4584:2:266:0.95:0.94:0.11:0.20), (Bursitis:216:4400:22:247:0.94:0.91:0.47:0.62), (Histi ocytosis.haematophagic:114:4502:4:265:0.94:0.97:0.30:0.46), (Ocular.discomfort:33:4583:0:269: 0.94:1.00:0.11:0.20), (Gastrointestinal.ulcer:25:4591:0:269:0.94:1.00:0.09:0.16), (Spinal.disorder: 174:4441:11:259:0.94:0.94:0.40:0.56), (Ear.disorder:120:4495:12:258:0.94:0.91:0.32:0.47), (Gast ric.bypass:79:4536:7:263:0.94:0.92:0.23:0.37), (Borderline.personality.disorder:66:4549:3:267:0. 94:0.96:0.20:0.33), (Pyoderma.gangrenosum:62:4553:0:270:0.94:1.00:0.19:0.31), (Anaphylaxis.pr ophylaxis:31:4584:0:270:0.94:1.00:0.10:0.19), (Pancreatitis.acute:23:4592:0:270:0.94:1.00:0.08: 0.15), (Thermal.bum:16:4599:0:270:0.94:1.00:0.06:0.11), (Renal.cancer.stage.IV:196:4418:35:23 6:0.94:0.85:0.45:0.59), (Pemphigus:47:4567:0:271:0.94:1.00:0.15:0.26), (Arthropod.sting:35:457 9:0:271:0.94:1.00:0.11:0.21), (Philadelphia.chromosome.positive:33:4581:0:271:0.94:1.00:0.11:0 0.20), (Streptococcal.infection:29:4585:0:271:0.94:1.00:0.10:0.18), (Zinc.deficiency:16:4598:0:271: 0.94:1.00:0.06:0.11), (Mitral.valve.prolapse:191:4422:18:254:0.94:0.91:0.43:0.58), (Suicidal.idea tion:127:4486:12:260:0.94:0.91:0.33:0.48), (Joint.injury:125:4488:9:263:0.94:0.93:0.32:0.48), (O steoporotic.fracture:125:4488:5:267:0.94:0.96:0.32:0.48), (Pericardial.effusion:74:4539:3:269:0.9 4:0.96:0.22:0.35), (Ischaemia:66:4547:1:271:0.94:0.99:0.20:0.33), (Neuromuscular.blocking.thera py:44:4569:2:270:0.94:0.96:0.14:0.24), (Blood.folate.decreased:25:4588:0:272:0.94:1.00:0.08:0. 16), (Cervix.carcinoma:36:4576:0:273:0.94:1.00:0.12:0.21), (Intertrigo:34:4578:0:273:0.94:1.00:0 0.11:0.20), (Brain.injury:32:4580:0:273:0.94:1.00:0.10:0.19), (Bone.density.abnormal:286:4325:28: 246:0.94:0.91:0.54:0.68), (Myeloid.leukaemia:122:4489:9:265:0.94:0.93:0.32:0.47), (Lung.carci noma.cell.type.unspecified.stage.0:81:4530:2:272:0.94:0.98:0.23:0.37), (Polycythaemia:65:4546: 3:271:0.94:0.96:0.19:0.32), (Postural.orthostatic.tachycardia.syndrome:58:4553:2:272:0.94:0.97: 0.18:0.30), (Oesophageal.ulcer:37:4574:0:274:0.94:1.00:0.12:0.21), (Urine.output.decreased:28:4 583:0:274:0.94:1.00:0.09:0.17), (Lymphoedema:136:4474:9:266:0.94:0.94:0.34:0.50), (Dysgeusia: 26:4584:0:275:0.94:1.00:0.09:0.16), (Rash.erythematous:26:4584:0:275:0.94:1.00:0.09:0.16), (R adiculopathy:152:4457:18:258:0.94:0.89:0.37:0.52), (Mixed.connective.tissue.disease:106:4503: 2:274:0.94:0.98:0.28:0.43), (Tension:91:4518:5:271:0.94:0.95:0.25:0.40), (Limb.injury:49:4560:1: 275:0.94:0.98:0.15:0.26), (Furuncle:30:4579:0:276:0.94:1.00:0.10:0.18), (Secondary.progressive. multiple.sclerosis:81:4527:1:276:0.94:0.99:0.23:0.37), (Adrenal.disorder:59:4549:1:276:0.94:0.9 8:0.18:0.30), (Rotator.cuff.syndrome:40:4568:1:276:0.94:0.98:0.13:0.22), (Type.V.hyperlipidaem ia:249:4358:29:249:0.94:0.90:0.50:0.64), (B-cell.small.lymphocytic.lymphoma:206:4401:29:249:0.94:0.88:0.45:0.60), (Intervertebral.disc.dis order:100:4507:5:273:0.94:0.95:0.27:0.42), (Malignant.respiratory.tract.neoplasm:93:4514:4:274: 0.94:0.96:0.25:0.40), (Insulin.resistance:83:4524:2:276:0.94:0.98:0.23:0.37), (Anaesthetic.premed ication:39:4568:2:276:0.94:0.95:0.12:0.22), (Temporal.arteritis:194:4412:20:259:0.94:0.91:0.43: 0.58), (Portal.hypertension:84:4522:1:278:0.94:0.99:0.23:0.38), (Pulmonary.mycosis:48:4558:1:2 78:0.94:0.98:0.15:0.26), (Lymphocytic.lymphoma:45:4561:1:278:0.94:0.98:0.14:0.24), (Blood.pr essure.fluctuation:41:4565:3:276:0.94:0.93:0.13:0.23), (Aphthous.ulcer:32:4574:0:279:0.94:1.00: 0.10:0.19), (Abdominal.pain.lower:24:4582:0:279:0.94:1.00:0.08:0.15), (Malignant.urinary.tract.n eoplasm:128:4477:14:266:0.94:0.90:0.32:0.48), (Lactose.intolerance:77:4528:1:279:0.94:0.99:0.2 2:0.35), (Prostatic.adenoma:71:4534:2:278:0.94:0.97:0.20:0.34), (Pneumonia.fungal:65:4540:0:28 0:0.94:1.00:0.19:0.32), (Blood.cholesterol.decreased:283:4321:36:245:0.94:0.89:0.54:0.67), (Inha lation.therapy:246:4358:31:250:0.94:0.89:0.50:0.64), (Neuroendocrine.carcinoma:139:4465:17:2 64:0.94:0.89:0.34:0.50), (Angioedema:118:4486:5:276:0.94:0.96:0.30:0.46), (Respiratory.therapy: 38:4566:0:281:0.94:1.00:0.12:0.21), (Balance.disorder:219:4384:25:257:0.94:0.90:0.46:0.61), (En terococcal.infection:87:4516:1:281:0.94:0.99:0.24:0.38), (Nasal.discomfort:56:4547:1:281:0.94:0 0.98:0.17:0.28), (Lung.carcinoma.cell.type.unspecified.stage.IV:46:4557:0:282:0.94:1.00:0.14:0.2 5), (Osteosarcoma:45:4558:0:282:0.94:1.00:0.14:0.24), (Urticaria.chronic:45:4558:0:282:0.94:1.0 0:0.14:0.24), (Rash.generalised:21:4582:0:282:0.94:1.00:0.07:0.13), (Low.density.lipoprotein.incr eased:271:4331:34:249:0.94:0.89:0.52:0.66), (Diabetic.foot:181:4421:17:266:0.94:0.91:0.40:0.56), (Heart.disease.congenital:123:4479:5:278:0.94:0.96:0.31:0.47), (Prostate.infection:60:4542:3:28 0:0.94:0.95:0.18:0.30), (Oral.infection:35:4567:0:283:0.94:1.00:0.11:0.20), (Circulatory.collapse: 26:4576:0:283:0.94:1.00:0.08:0.16), (Urosepsis:26:4576:0:283:0.94:1.00:0.08:0.16), (Urine.analy sis.abnormal:19:4583:0:283:0.94:1.00:0.06:0.12), (Fabry's.disease:276:4325:34:250:0.94:0.89:0.5 2:0.66), (Sexual.dysfunction:147:4454:15:269:0.94:0.91:0.35:0.51), (Kidney.infection:137:4464:1 3:271:0.94:0.91:0.34:0.49), (Hemia:122:4479:10:274:0.94:0.92:0.31:0.46), (Medullary.thyroid.ca ncer:32:4569:0:284:0.94:1.00:0.10:0.18), (Scleroderma.associated.digital.ulcer:29:4572:0:284:0.9 4:1.00:0.09:0.17), (Drug.withdrawal.syndrome:26:4575:0:284:0.94:1.00:0.08:0.15), (Oestrogen.re placement.therapy:205:4395:32:253:0.94:0.86:0.45:0.59), (Insulin-requiring.type.2.diabetes.mellitus:179:4421:27:258:0.94:0.87:0.41:0.56), (Muscle.tightness:171:4 429:22:263:0.94:0.89:0.39:0.55), (Macular.oedema:152:4448:16:269:0.94:0.90:0.36:0.52), (Idiopa thic.urticaria:121:4479:13:272:0.94:0.90:0.31:0.46), (Extrasystoles:82:4518:5:280:0.94:0.94:0.23: 0.37), (Humoral.immune.defect:57:4543:0:285:0.94:1.00:0.17:0.29), (Ovarian.cancer.recurrent:2 8:4572:0:285:0.94:1.00:0.09:0.16), (Fracture.pain:25:4575:0:285:0.94:1.00:0.08:0.15), (Torticollis: 116:4483:11:275:0.94:0.91:0.30:0.45), (T-cell.lymphoma:71:4528:2:284:0.94:0.97:0.20:0.33), (Rectosigmoid.cancer:59:4540:1:285:0.94:0. 98:0.17:0.29), (Pericarditis:55:4544:0:286:0.94:1.00:0.16:0.28), (Blood.phosphorus.decreased:35: 4564:0:286:0.94:1.00:0.11:0.20), (Autonomic.nervous.system.imbalance:144:4454:12:275:0.94:0 0.92:0.34:0.50), (Throat.irritation:41:4557:0:287:0.94:1.00:0.13:0.22), (Prostatic.specific.antigen.in creased:26:4572:0:287:0.94:1.00:0.08:0.15), (Ocular.hyperaemia:19:4579:0:287:0.94:1.00:0.06:0 0.12), (Cystitis.interstitial:323:4274:49:239:0.94:0.87:0.57:0.69), (Angiomyolipoma:97:4500:6:282: 0.94:0.94:0.26:0.40), (Cystic.fibrosis.lung:52:4545:2:286:0.94:0.96:0.15:0.27), (Ligament.sprain: 37:4560:0:288:0.94:1.00:0.11:0.20), (Colorectal.adenocarcinoma:25:4572:0:288:0.94:1.00:0.08:0 0.15), (Faeces.soft:238:4358:32:257:0.94:0.88:0.48:0.62), (Movement.disorder:134:4462:15:274:0. 94:0.90:0.33:0.48), (Disease.progression:128:4468:6:283:0.94:0.96:0.31:0.47), (Cerebral.palsy:64: 4532:0:289:0.94:1.00:0.18:0.31), (Nicotine.dependence:64:4532:3:286:0.94:0.96:0.18:0.31), (Kid ney.transplant.rejection:59:4537:0:289:0.94:1.00:0.17:0.29), (Cyst:45:4551:1:288:0.94:0.98:0.14: 0.24), (Enzyme.supplementation:32:4564:0:289:0.94:1.00:0.10:0.18), (Psychiatric.symptom:32:45 64:2:287:0.94:0.94:0.10:0.18), (Persistent.depressive.disorder:26:4570:0:289:0.94:1.00:0.08:0.15), (Carpal.tunnel.syndrome:158:4437:19:271:0.94:0.89:0.37:0.52), (Chronic.myelomonocytic.leuk aemia:107:4488:5:285:0.94:0.96:0.27:0.42), (Bladder.pain:101:4494:11:279:0.94:0.90:0.27:0.41), (Pharyngitis.streptococcal:54:4541:0:290:0.94:1.00:0.16:0.27), (Pituitary.tumour.benign:43:4552: 0:290:0.94:1.00:0.13:0.23), (Soft.tissue.infection:33:4562:0:290:0.94:1.00:0.10:0.19), (Anal.fissur e:23:4572:0:290:0.94:1.00:0.07:0.14), (Atrophic.vulvovaginitis:188:4406:21:270:0.94:0.90:0.41: 0.56), (Skin.cancer:181:4413:13:278:0.94:0.93:0.39:0.55), (Arterial.disorder:175:4419:14:277:0.9 4:0.93:0.39:0.55), (Matemal.exposure.timing.unspecified:129:4465:3:288:0.94:0.98:0.31:0.47), (P latelet.aggregation.inhibition:129:4465:8:283:0.94:0.94:0.31:0.47), (Paranoia:95:4499:6:285:0.94: 0.94:0.25:0.40), (Hypertrophic.cardiomyopathy:93:4501:6:285:0.94:0.94:0.25:0.39), (Limb.disco mfort:297:4296:38:254:0.94:0.89:0.54:0.67), (Renal.failure.chronic:125:4468:11:281:0.94:0.92:0 0.31:0.46), (Postoperative.wound.infection:37:4556:0:292:0.94:1.00:0.11:0.20), (Oesophageal.spas m:144:4448:19:274:0.94:0.88:0.34:0.50), (Lymphocytic.leukaemia:119:4473:8:285:0.94:0.94:0.2 9:0.45), (Atrial.tachycardia:102:4490:9:284:0.94:0.92:0.26:0.41), (Clear.cell.renal.cell.carcinoma: 94:4498:8:285:0.94:0.92:0.25:0.39), (Infarction:92:4500:6:287:0.94:0.94:0.24:0.39), (Antioxidant. therapy:56:4536:0:293:0.94:1.00:0.16:0.28), (Blister:53:4539:0:293:0.94:1.00:0.15:0.27), (Person ality.disorder:50:4542:2:291:0.94:0.96:0.15:0.25), (Procedural.nausea:34:4558:0:293:0.94:1.00:0. 10:0.19), (Blood.uric.acid.abnormal:162:4429:21:273:0.94:0.89:0.37:0.52), (Diverticulum:115:44 76:7:287:0.94:0.94:0.29:0.44), (Dwarfism:71:4520:2:292:0.94:0.97:0.20:0.33), (Small.intestine.ca rcinoma:43:4548:0:294:0.94:1.00:0.13:0.23), (Upper.gastrointestinal.haemorrhage:29:4562:0:294: 0.94:1.00:0.09:0.16), (Disturbance.in.attention:182:4408:19:276:0.94:0.91:0.40:0.55), (Bone.mar row.transplant:105:4485:1:294:0.94:0.99:0.26:0.42), (Pain.in.jaw:61:4529:1:294:0.94:0.98:0.17:0 0.29), (Lymphadenopathy:53:4537:0:295:0.94:1.00:0.15:0.26), (Acute.respiratory.failure:49:4541:1: 294:0.94:0.98:0.14:0.25), (Lung.transplant:220:4369:21:275:0.94:0.91:0.44:0.60), (Bronchial.hyp erreactivity:211:4378:22:274:0.94:0.91:0.44:0.59), (Menstrual.cycle.management:169:4420:19:2 77:0.94:0.90:0.38:0.53), (Muscle.twitching:92:4497:3:293:0.94:0.97:0.24:0.38), (Inflammatory.pa in:76:4513:4:292:0.94:0.95:0.21:0.34), (Leukocytosis:70:4519:0:296:0.94:1.00:0.19:0.32), (Gastr ointestinal.infection:28:4561:0:296:0.94:1.00:0.09:0.16), (Vitamin.B12.decreased:228:4360:31:2 66:0.94:0.88:0.46:0.61), (Hip.arthroplasty:211:4377:27:270:0.94:0.89:0.44:0.59), (Hepatic.enzym e.increased:35:4553:0:297:0.94:1.00:0.11:0.19), (Rib.fracture:19:4569:0:297:0.94:1.00:0.06:0.11), (Biopsy.bone.marrow:16:4572:0:297:0.94:1.00:0.05:0.10), (Tooth.disorder:184:4403:18:280:0.9 4:0.91:0.40:0.55), (Fracture:160:4427:14:284:0.94:0.92:0.36:0.52), (Urge.incontinence:135:4452: 12:286:0.94:0.92:0.32:0.48), (Facial.pain:94:4493:6:292:0.94:0.94:0.24:0.39), (Food.allergy:80:4 507:4:294:0.94:0.95:0.21:0.35), (Cachexia:32:4555:0:298:0.94:1.00:0.10:0.18), (Inappropriate.ant idiuretic.hormone.secretion:29:4558:0:298:0.94:1.00:0.09:0.16), (Visual.impairment:163:4423:13: 286:0.94:0.93:0.36:0.52), (Abdominal.infection:23:4563:0:299:0.94:1.00:0.07:0.13), (General.ph ysical.condition:176:4409:21:279:0.94:0.89:0.39:0.54), (Poor.quality.sleep:149:4436:11:289:0.94: 0.93:0.34:0.50), (Guillain-Barre.syndrome:55:4530:0:300:0.94:1.00:0.15:0.27), (Loss.of.consciousness:33:4552:0:300:0.94: 1.00:0.10:0.18), (Oral.disorder:30:4555:0:300:0.94:1.00:0.09:0.17), (Nephropathy:268:4316:28:2 73:0.94:0.91:0.50:0.64), (Bone.loss:253:4331:37:264:0.94:0.87:0.49:0.63), (Relaxation.therapy:17 5:4409:25:276:0.94:0.88:0.39:0.54), (Skin.irritation:151:4433:13:288:0.94:0.92:0.34:0.50), (Bipol ar.II.disorder:101:4483:5:296:0.94:0.95:0.25:0.40), (Secondary.immunodeficiency:52:4532:0:301: 0.94:1.00:0.15:0.26), (Systemic.candida:41:4543:0:301:0.94:1.00:0.12:0.21), (Osteitis:40:4544:0: 301:0.94:1.00:0.12:0.21), (5q.minus.syndrome:222:4361:40:262:0.94:0.85:0.46:0.60), (Ischaemic. cardiomyopathy:218:4365:20:282:0.94:0.92:0.44:0.59), (Cor.pulmonale.chronic:307:4275:41:262: 0.94:0.88:0.54:0.67), (Thyroid.hormone.replacement.therapy:283:4299:42:261:0.94:0.87:0.52:0. 65), (Cardiac.valve.disease:213:4369:30:273:0.94:0.88:0.44:0.58), (Apnoea:114:4468:16:287:0.94: 0.88:0.28:0.43), (Skin.test:60:4522:4:299:0.94:0.94:0.17:0.28), (Amnesia:272:4309:39:265:0.94: 0.87:0.51:0.64), (Autonomic.neuropathy:262:4319:35:269:0.94:0.88:0.49:0.63), (Blood.potassium: 262:4319:35:269:0.94:0.88:0.49:0.63), (Musculoskeletal.discomfort:251:4330:35:269:0.94:0.88: 0.48:0.62), (Nasal.disorder:135:4446:18:286:0.94:0.88:0.32:0.47), (Red.blood.cell.count.decrease d:85:4496:2:302:0.94:0.98:0.22:0.36), (Complex.partial.seizures:81:4500:2:302:0.94:0.98:0.21:0. 35), (Portal.vein.thrombosis:57:4524:0:304:0.94:1.00:0.16:0.27), (International.normalised.ratio.i ncreased:29:4552:0:304:0.94:1.00:0.09:0.16), (Connective.tissue.disorder:246:4334:43:262:0.94: 0.85:0.48:0.62), (Soft.tissue.sarcoma:237:4343:26:279:0.94:0.90:0.46:0.61), (Magnesium.deficien cy:177:4403:20:285:0.94:0.90:0.38:0.54), (Autoimmune.haemolytic.anaemia:139:4441:8:297:0.9 4:0.95:0.32:0.48), (Pituitary-dependent.Cushing's.syndrome:135:4445:10:295:0.94:0.93:0.31:0.47), (Oesophageal.adenocarcin oma:32:4548:0:305:0.94:1.00:0.09:0.17), (Burning.sensation:307:4272:35:271:0.94:0.90:0.53:0.6 7), (Proteinuria:249:4330:29:277:0.94:0.90:0.47:0.62), (Pemicious.anaemia:194:4385:20:286:0.94: 0.91:0.40:0.56), (Breast.cancer.stage.IV:164:4415:18:288:0.94:0.90:0.36:0.52), (Encephalopathy: 167:4411:12:295:0.94:0.93:0.36:0.52), (Arthritis.bacterial:54:4524:0:307:0.94:1.00:0.15:0.26), (N euromuscular.blockade.reversal:49:4529:1:306:0.94:0.98:0.14:0.24), (Hidradenitis:289:4288:43:2 65:0.94:0.87:0.52:0.65), (Obstructive.airways.disorder:221:4356:29:279:0.94:0.88:0.44:0.59), (Hy perammonaemia:26:4551:0:308:0.94:1.00:0.08:0.14), (Pulmonary.fibrosis:382:4194:54:255:0.94: 0.88:0.60:0.71), (Eye.inflammation:119:4457:4:305:0.94:0.97:0.28:0.44), (Mycobacterium.avium. complex.infection:83:4493:0:309:0.94:1.00:0.21:0.35), (Poor.peripheral.circulation:77:4499:4:30 5:0.94:0.95:0.20:0.33), (Chronic.inflammatory.demyelinating.polyradiculoneuropathy:285:4290: 38:272:0.94:0.88:0.51:0.65), (Tardive.dyskinesia:245:4330:37:273:0.94:0.87:0.47:0.61), (Sickle.c ell.anaemia:189:4386:17:293:0.94:0.92:0.39:0.55), (Eye.irritation:104:4471:8:302:0.94:0.93:0.26: 0.40), (Congestive.cardiomyopathy:93:4482:2:308:0.94:0.98:0.23:0.38), (Thrombophlebitis:81:4 494:1:309:0.94:0.99:0.21:0.34), (Prostate.cancer.stage.IV:57:4518:0:310:0.94:1.00:0.16:0.27), (Li ver.function.test.abnormal:47:4528:1:309:0.94:0.98:0.13:0.23), (Compression.fracture:39:4536:0: 310:0.94:1.00:0.11:0.20), (Syncope:278:4296:36:275:0.94:0.89:0.50:0.64), (Hysterectomy:266:43 08:36:275:0.94:0.88:0.49:0.63), (Latent.tuberculosis:99:4475:1:310:0.94:0.99:0.24:0.39), (Hormo ne.level.abnormal:323:4250:46:266:0.94:0.88:0.55:0.67), (Hair.disorder:254:4319:33:279:0.94:0. 89:0.48:0.62), (Hypertensive.heart.disease:177:4396:19:293:0.94:0.90:0.38:0.53), (Lipid.metaboli sm.disorder:136:4437:12:300:0.94:0.92:0.31:0.47), (Endometrial.cancer:49:4524:0:312:0.94:1.00: 0.14:0.24), (Arteriosclerosis.coronary.artery:205:4367:18:295:0.94:0.92:0.41:0.57), (Addison's.di sease:176:4396:28:285:0.94:0.86:0.38:0.53), (Psychomotor.hyperactivity:140:4432:15:298:0.94:0 0.90:0.32:0.47), (Haemodialysis:44:4528:0:313:0.94:1.00:0.12:0.22), (Sputum.retention:22:4550:0: 313:0.94:1.00:0.07:0.12), (Nail.disorder:240:4331:35:279:0.94:0.87:0.46:0.60), (Dementia.with.L ewy.bodies:134:4437:10:304:0.94:0.93:0.31:0.46), (Menstrual.disorder:98:4473:3:311:0.94:0.97: 0.24:0.38), (Thrombocytosis:93:4478:5:309:0.94:0.95:0.23:0.37), (Paralysis:52:4519:2:312:0.94:0 0.96:0.14:0.25), (Oedema.due.to.cardiac.disease:23:4548:1:313:0.94:0.96:0.07:0.13), (Appetite.dis order:343:4227:53:262:0.94:0.87:0.57:0.69), (Diabetic.retinopathy:215:4355:30:285:0.94:0.88:0. 43:0.58), (Blood.calcium:213:4357:26:289:0.94:0.89:0.42:0.57), (Granulomatosis.with.polyangiiti s:186:4384:16:299:0.94:0.92:0.38:0.54), (Metastases.to.lymph.nodes:186:4384:15:300:0.94:0.93: 0.38:0.54), (Dystonia:184:4386:24:291:0.94:0.88:0.39:0.54), (Hair.growth.abnormal:177:4393:23: 292:0.94:0.89:0.38:0.53), (Otitis.media:132:4438:6:309:0.94:0.96:0.30:0.46), (Groin.pain:46:4524:0:315:0.94:1.00:0.13:0.23), (Selective.IgG.subclass.deficiency:307:4262:50:266:0.94:0.86:0.54: 0.66), (Primary.progressive.multiple.sclerosis:28:4541:0:316:0.94:1.00:0.08:0.15), (Circadian.rhyt hm.sleep.disorder:322:4246:55:262:0.94:0.85:0.55:0.67), (Antiretroviral.therapy:236:4332:22:29 5:0.94:0.91:0.44:0.60), (Adenocarcinoma:213:4355:25:292:0.94:0.89:0.42:0.57), (Affect.lability:1 92:4376:26:291:0.94:0.88:0.40:0.55), (Injury:121:4447:17:300:0.94:0.88:0.29:0.43), (Malabsorpti on:116:4452:5:312:0.94:0.96:0.27:0.42), (Uterine.cancer:102:4466:5:312:0.94:0.95:0.25:0.39), (In itial.insomnia:94:4474:5:312:0.94:0.95:0.23:0.37), (Oral.fungal.infection:53:4515:1:316:0.94:0.9 8:0.14:0.25), (Gastroduodenal.ulcer:41:4527:0:317:0.94:1.00:0.11:0.21), (Hereditary.angioedema: 391:4176:52:266:0.93:0.88:0.60:0.71), (Abnormal.faeces:314:4253:54:264:0.93:0.85:0.54:0.66), (Gaucher's.disease:160:4407:21:297:0.93:0.88:0.35:0.50), (Night.sweats:149:4418:16:302:0.93:0. 90:0.33:0.48), (Premenstrual.syndrome:221:4345:22:297:0.93:0.91:0.43:0.58), (Sarcoma:156:441 0:11:308:0.93:0.93:0.34:0.49), (Abscess:151:4415:14:305:0.93:0.92:0.33:0.49), (Prophylaxis.agai nst.HIV.infection:87:4479:2:317:0.93:0.98:0.22:0.35), (Completed.suicide:50:4516:4:315:0.93:0. 93:0.14:0.24), (Blood.sodium.decreased:33:4533:0:319:0.93:1.00:0.09:0.17), (Lung.adenocarcino ma.metastatic:32:4534:0:319:0.93:1.00:0.09:0.17), (Retinal.vein.occlusion:198:4367:22:298:0.93: 0.90:0.40:0.55), (Haematological.malignancy:171:4394:13:307:0.93:0.93:0.36:0.52), (Acute.lym phocytic.leukaemia.recurrent:97:4468:3:317:0.93:0.97:0.23:0.38), (Metastatic.gastric.cancer:40:4 525:0:320:0.93:1.00:0.11:0.20), (Goitre:200:4364:21:300:0.93:0.90:0.40:0.55), (Percutaneous.cor onary.intervention:136:4428:20:301:0.93:0.87:0.31:0.46), (Vasodilatation:114:4450:4:317:0.93:0 0.97:0.26:0.42), (HIV.test.positive:95:4469:3:318:0.93:0.97:0.23:0.37), (Klebsiella.infection:71:44 93:2:319:0.93:0.97:0.18:0.31), (Complex.regional.pain.syndrome:291:4272:38:284:0.93:0.88:0.5 1:0.64), (Gastrointestinal.pain:237:4326:26:296:0.93:0.90:0.44:0.60), (Hyperparathyroidism:195: 4368:16:306:0.93:0.92:0.39:0.55), (Staphylococcal.bacteraemia:77:4486:2:320:0.93:0.97:0.19:0. 32), (Central.venous.catheterisation:40:4523:0:322:0.93:1.00:0.11:0.20), (Shock:38:4525:0:322:0. 93:1.00:0.11:0.19), (Actinic.keratosis:256:4306:35:288:0.93:0.88:0.47:0.61), (Vitamin.D:210:435 2:27:296:0.93:0.89:0.42:0.57), (Antiphospholipid.syndrome:204:4358:18:305:0.93:0.92:0.40:0.5 6), (Blood.uric.acid:161:4401:17:306:0.93:0.90:0.34:0.50), (Ear.pain:152:4410:14:309:0.93:0.92: 0.33:0.48), (Pancreatitis.chronic:149:4413:16:307:0.93:0.90:0.33:0.48), (Tachyarrhythmia:38:452 4:0:323:0.93:1.00:0.11:0.19), (Autoimmune.disorder:340:4221:44:280:0.93:0.89:0.55:0.68), (Met astasis:309:4252:44:280:0.93:0.88:0.52:0.66), (Fluid.imbalance:242:4319:24:300:0.93:0.91:0.45: 0.60), (Medical.diet:171:4390:21:303:0.93:0.89:0.36:0.51), (Cerebral.disorder:134:4427:10:314:0. 93:0.93:0.30:0.45), (Myoclonus:63:4498:2:322:0.93:0.97:0.16:0.28), (Flank.pain:46:4515:1:323:0 0.93:0.98:0.12:0.22), (Sarcoidosis:406:4154:65:260:0.93:0.86:0.61:0.71), (Tooth.infection:155:440 5:11:314:0.93:0.93:0.33:0.49), (Blood.triglycerides:381:4178:61:265:0.93:0.86:0.59:0.70), (Dyski nesia:277:4282:34:292:0.93:0.89:0.49:0.63), (Coronary.artery.bypass:217:4342:24:302:0.93:0.90: 0.42:0.57), (Hepatic.steatosis:130:4429:11:315:0.93:0.92:0.29:0.44), (Autoimmune.hepatitis:83:4 476:0:326:0.93:1.00:0.20:0.34), (Gastritis.erosive:74:4485:2:324:0.93:0.97:0.19:0.31), (Analgesic.intervention.supportive.therapy:53:4506:1:325:0.93:0.98:0.14:0.25), (Encephalitis:49:4510:0:32 6:0.93:1.00:0.13:0.23), (Asthma.prophylaxis:38:4521:0:326:0.93:1.00:0.10:0.19), (Vision.blurred: 37:4522:0:326:0.93:1.00:0.10:0.19), (Micturition.disorder:263:4295:31:296:0.93:0.89:0.47:0.62), (Upper-airway.cough.syndrome:217:4341:30:297:0.93:0.88:0.42:0.57), (Lung.cancer.metastatic:183:437 5:21:306:0.93:0.90:0.37:0.53), (Platelet.disorder:162:4396:18:309:0.93:0.90:0.34:0.50), (Genital.h erpes:182:4375:16:312:0.93:0.92:0.37:0.53), (Ocular.hypertension:133:4424:8:320:0.93:0.94:0.2 9:0.45), (Chronic.spontaneous.urticaria:128:4429:13:315:0.93:0.91:0.29:0.44), (Primary.myelofib rosis:107:4450:8:320:0.93:0.93:0.25:0.39), (Vulvovaginal.mycotic.infection:104:4453:8:320:0.93: 0.93:0.25:0.39), (Wound.treatment:44:4513:1:327:0.93:0.98:0.12:0.21), (Uterine.leiomyoma:236: 4320:26:303:0.93:0.90:0.44:0.59), (Diabetes.mellitus.inadequate.control:224:4332:22:307:0.93:0. 91:0.42:0.58), (Bronchial.carcinoma:215:4341:31:298:0.93:0.87:0.42:0.57), (Cerebrovascular.diso rder:98:4458:4:325:0.93:0.96:0.23:0.37), (Glycogen.storage.disease.type.II:48:4508:0:329:0.93:1. 00:0.13:0.23), (Pruritus.generalised:24:4532:0:329:0.93:1.00:0.07:0.13), (Blood.potassium.abnor mal:309:4246:49:281:0.93:0.86:0.52:0.65), (Bone.density.decreased:222:4333:24:306:0.93:0.90: 0.42:0.57), (Tendonitis:164:4391:12:318:0.93:0.93:0.34:0.50), (Tooth.abscess:100:4455:6:324:0.9 3:0.94:0.24:0.38), (Carotid.artery.stenosis:107:4447:7:324:0.93:0.94:0.25:0.39), (Carcinoid.syndr ome:316:4237:50:282:0.93:0.86:0.53:0.66), (Chronic.fatigue.syndrome:173:4380:21:311:0.93:0.8 9:0.36:0.51), (Cataract.operation:172:4381:14:318:0.93:0.92:0.35:0.51), (Sinus.headache:102:445 1:12:320:0.93:0.89:0.24:0.38), (Bronchial.disorder:96:4457:10:322:0.93:0.91:0.23:0.37), (Varicos e.vein:86:4467:3:329:0.93:0.97:0.21:0.34), (Polycystic.ovaries:336:4215:50:284:0.93:0.87:0.54:0 0.67), (Knee.arthroplasty:254:4297:40:294:0.93:0.86:0.46:0.60), (Metastatic.neoplasm:242:4309:2 7:307:0.93:0.90:0.44:0.59), (Drug.use.disorder:155:4396:9:325:0.93:0.95:0.32:0.48), (Arthropod. bite:51:4500:2:332:0.93:0.96:0.13:0.23), (Cholangitis:28:4523:0:334:0.93:1.00:0.08:0.14), (Perip heral.T-cell.lymphoma.unspecified:78:4472:3:332:0.93:0.96:0.19:0.32), (Overdose:62:4488:4:331:0.93:0. 94:0.16:0.27), (Neurosis:28:4522:0:335:0.93:1.00:0.08:0.14), (Muscle.disorder:302:4247:54:282: 0.93:0.85:0.52:0.64), (Lipids.abnormal:245:4304:25:311:0.93:0.91:0.44:0.59), (Cardiovascular.ins ufficiency:51:4498:0:336:0.93:1.00:0.13:0.23), (Carcinoid.tumour:298:4250:41:296:0.93:0.88:0.5 0:0.64), (Amyloidosis:262:4286:42:295:0.93:0.86:0.47:0.61), (Myositis:212:4336:33:304:0.93:0.8 7:0.41:0.56), (Skin.wrinkling:199:4349:35:302:0.93:0.85:0.40:0.54), (Condition.aggravated:189:4 359:24:313:0.93:0.89:0.38:0.53), (Respiratory.distress:189:4359:25:312:0.93:0.88:0.38:0.53), (Pa ncreatic.failure:44:4504:0:337:0.93:1.00:0.12:0.21), (Blood.triglycerides.abnormal:327:4220:60: 278:0.93:0.84:0.54:0.66), (Pemphigoid:102:4445:4:334:0.93:0.96:0.23:0.38), (Accidental.exposur e.to.product:64:4483:1:337:0.93:0.98:0.16:0.27), (Vulvovaginal.candidiasis:43:4504:0:338:0.93:1 0.00:0.11:0.20), (Skin.exfoliation:42:4505:0:338:0.93:1.00:0.11:0.20), (Alanine.aminotransferase.i ncreased:29:4518:0:338:0.93:1.00:0.08:0.15), (Cushing's.syndrome:381:4165:57:282:0.93:0.87:0. 57:0.69), (Uveitis:282:4264:31:308:0.93:0.90:0.48:0.62), (Nocturia:239:4307:31:308:0.93:0.89:0. 44:0.59), (Pelvic.pain:230:4316:32:307:0.93:0.88:0.43:0.58), (Tension.headache:166:4380:16:323: 0.93:0.91:0.34:0.49), (Chronic.hepatitis:42:4504:0:339:0.93:1.00:0.11:0.20), (Periarthritis:24:452 2:0:339:0.93:1.00:0.07:0.12), (Myeloproliferative.neoplasm:261:4284:43:297:0.93:0.86:0.47:0.61), (Heart.transplant:240:4305:21:319:0.93:0.92:0.43:0.59), (Increased.appetite:178:4367:22:318:0. 93:0.89:0.36:0.51), (Escherichia.infection:133:4412:10:330:0.93:0.93:0.29:0.44), (Post.procedural.infection:77:4468:3:337:0.93:0.96:0.19:0.31), (Brain.neoplasm:226:4318:24:317:0.93:0.90:0.42: 0.57), (Eye.pruritus:213:4331:24:317:0.93:0.90:0.40:0.56), (Heart.rate.abnormal:365:4178:61:281: 0.93:0.86:0.57:0.68), (Weight.increased:283:4259:38:305:0.93:0.88:0.48:0.62), (Hyperhidrosis:1 75:4367:14:329:0.93:0.93:0.35:0.51), (Post.procedural.hypothyroidism:46:4496:0:343:0.93:1.00: 0.12:0.21), (Eye.lubrication.therapy:44:4498:1:342:0.93:0.98:0.11:0.20), (Bone.cancer:382:4159: 65:279:0.93:0.85:0.58:0.69), (Dental.care:209:4332:26:318:0.93:0.89:0.40:0.55), (Hypoparathyroi dism:120:4421:6:338:0.93:0.95:0.26:0.41), (Tooth.extraction:90:4451:2:342:0.93:0.98:0.21:0.34), (Procoagulant.therapy:35:4506:0:344:0.93:1.00:0.09:0.17), (Coronary.arterial.stent.insertion:301: 4239:38:307:0.93:0.89:0.50:0.64), (Body.temperature.increased:157:4383:15:330:0.93:0.91:0.32: 0.48), (Adenocarcinoma.of.colon:93:4447:6:339:0.93:0.94:0.22:0.35), (Triple.negative.breast.can cer:66:4474:5:340:0.93:0.93:0.16:0.28), (Androgen.replacement.therapy:425:4114:66:280:0.93:0. 87:0.60:0.71), (Epigastric.discomfort:178:4361:21:325:0.93:0.89:0.35:0.51), (Erysipelas:169:4370: 18:328:0.93:0.90:0.34:0.49), (Metabolic.acidosis:96:4443:3:343:0.93:0.97:0.22:0.36), (Blood.cre atinine.increased:52:4487:0:346:0.93:1.00:0.13:0.23), (Hypogonadism:408:4130:67:280:0.93:0.8 6:0.59:0.70), (Flushing:290:4248:37:310:0.93:0.89:0.48:0.63), (Secretion.discharge:285:4253:41: 306:0.93:0.87:0.48:0.62), (Transplant:278:4260:34:313:0.93:0.89:0.47:0.62), (Feeling.of.relaxatio n:235:4303:44:303:0.93:0.84:0.44:0.58), (Cholangiocarcinoma:145:4393:18:329:0.93:0.89:0.31:0 0.46), (Peritonitis:63:4475:0:347:0.93:1.00:0.15:0.27), (Antidiarrhoeal.supportive.care:29:4509:0:3 47:0.93:1.00:0.08:0.14), (Transplant.rejection:295:4242:37:311:0.93:0.89:0.49:0.63), (Blood.gluc ose.decreased:245:4292:33:315:0.93:0.88:0.44:0.58), (Huntington's.disease:194:4343:33:315:0.9 3:0.85:0.38:0.53), (Bone.marrow.failure:106:4431:6:342:0.93:0.95:0.24:0.38), (Congenital.cystic. kidney.disease:87:4450:1:347:0.93:0.99:0.20:0.33), (Infusion:31:4506:0:348:0.93:1.00:0.08:0.15), (Acute.sinusitis:142:4394:9:340:0.93:0.94:0.29:0.45), (Pneumonitis:247:4288:40:310:0.93:0.86: 0.44:0.59), (Aggression:220:4315:25:325:0.93:0.90:0.40:0.56), (Contusion:110:4425:7:343:0.93:0 0.94:0.24:0.39), (Opportunistic.infection.prophylaxis:107:4428:4:346:0.93:0.96:0.24:0.38), (Influe nza.immunisation:53:4481:0:351:0.93:1.00:0.13:0.23), (Immune.enhancement.therapy:43:4491:0:351:0.93:1.00:0.11:0.20), (C-reactive.protein.increased:38:4496:0:351:0.93:1.00:0.10:0.18), (End.stage.renal.disease:299:4234:43:309:0.93:0.87:0.49:0.63), (Muscular.weakness:245:4288:38:314:0.93:0.87:0.44:0.58), (Aortic. valve.replacement:221:4312:20:332:0.93:0.92:0.40:0.56), (Acquired.immunodeficiency.syndrom e:175:4358:5:347:0.93:0.97:0.34:0.50), (Endocarditis:146:4387:9:343:0.93:0.94:0.30:0.45), (Diab etes.insipidus:87:4446:4:348:0.93:0.96:0.20:0.33), (Blood.potassium.increased:266:4266:38:315: 0.93:0.88:0.46:0.60), (Evidence.based.treatment:185:4347:9:344:0.93:0.95:0.35:0.51), (Endotrach eal.intubation:128:4404:12:341:0.93:0.91:0.27:0.42), (Hepatic.failure:77:4455:3:350:0.93:0.96:0. 18:0.30), (Breast.cancer.recurrent:65:4467:1:352:0.93:0.98:0.16:0.27), (Intervertebral.disc.degene ration:403:4128:59:295:0.93:0.87:0.58:0.69), (Pregnancy:232:4299:18:336:0.93:0.93:0.41:0.57), (Abnormal.behaviour:221:4310:27:327:0.93:0.89:0.40:0.56), (Product.use.in.unapproved.indicatio n:128:4403:7:347:0.93:0.95:0.27:0.42), (Salivary.hypersecretion:98:4433:5:349:0.93:0.95:0.22:0. 36), (Varices.oesophageal:77:4454:1:353:0.93:0.99:0.18:0.30), (Confusional.state:190:4340:26:32 9:0.93:0.88:0.37:0.52), (Hypertonia:117:4413:6:349:0.93:0.95:0.25:0.40), (Papillary.thyroid.cance r:86:4444:4:351:0.93:0.96:0.20:0.33), (Osteonecrosis:51:4479:1:354:0.93:0.98:0.13:0.22), (Parkin son's.disease.psychosis:383:4146:63:293:0.93:0.86:0.57:0.68), (Oesophageal.carcinoma:246:428 3:38:318:0.93:0.87:0.44:0.58), (Head.and.neck.cancer:86:4443:7:349:0.93:0.92:0.20:0.33), (Dysb acteriosis:38:4491:0:356:0.93:1.00:0.10:0.18), (Migraine.prophylaxis:346:4182:46:311:0.93:0.88: 0.53:0.66), (Diabetic.nephropathy:256:4272:31:326:0.93:0.89:0.44:0.59), (Vitamin.B.complex.de ficiency:241:4287:34:323:0.93:0.88:0.43:0.57), (Polymyositis:199:4329:22:335:0.93:0.90:0.37:0. 53), (Malignant.peritoneal.neoplasm:143:4385:15:342:0.93:0.91:0.29:0.44), (Lung.adenocarcinom a.stage.IV:119:4409:11:346:0.93:0.92:0.26:0.40), (Leukaemia:392:4135:46:312:0.93:0.89:0.56:0. 69), (Biliary.cirrhosis.primary:249:4278:32:326:0.93:0.89:0.43:0.58), (Squamous.cell.carcinoma: 256:4270:36:323:0.93:0.88:0.44:0.59), (Palliative.care:84:4442:4:355:0.93:0.95:0.19:0.32), (Acut e.graft.versus.host.disease:53:4473:0:359:0.93:1.00:0.13:0.23), (Hyperkeratosis:30:4496:0:359:0. 93:1.00:0.08:0.14), (Drug.hypersensitivity:262:4263:35:325:0.93:0.88:0.45:0.59), (Folliculitis:144: 4381:14:346:0.93:0.91:0.29:0.44), (Meningitis:66:4459:1:359:0.93:0.99:0.16:0.27), (Staphylococ cal.sepsis:33:4492:0:360:0.93:1.00:0.08:0.15), (Tinea.infection:33:4492:1:359:0.93:0.97:0.08:0.1 5), (Brain.neoplasm.malignant:365:4159:54:307:0.93:0.87:0.54:0.67), (Ex-tobacco.user:348:4176:50:311:0.93:0.87:0.53:0.66), (Generalised.anxiety.disorder:270:4254:38:3 23:0.93:0.88:0.46:0.60), (Ventricular.extrasystoles:245:4279:27:334:0.93:0.90:0.42:0.58), (Periph eral.venous.disease:201:4323:16:345:0.93:0.93:0.37:0.53), (Transitional.cell.carcinoma:145:4379:13:348:0.93:0.92:0.29:0.45), (Lyme.disease:73:4451:3:358:0.93:0.96:0.17:0.29), (Intraocular.pre ssure.increased:304:4219:48:314:0.93:0.86:0.49:0.63), (Prophylactic.chemotherapy:300:4223:28: 334:0.93:0.91:0.47:0.62), (Angiopathy:277:4246:42:320:0.93:0.87:0.46:0.60), (Hepatitis:183:434 0:14:348:0.93:0.93:0.34:0.50), (Bone.lesion:90:4433:1:361:0.93:0.99:0.20:0.33), (Intestinal.obstru ction:72:4451:0:362:0.93:1.00:0.17:0.28), (Probiotic.therapy:366:4156:59:304:0.93:0.86:0.55:0.6 7), (Essential.tremor:316:4206:48:315:0.93:0.87:0.50:0.64), (Vaginal.infection:148:4374:9:354:0. 93:0.94:0.29:0.45), (Blood.iron.decreased:417:4104:66:298:0.93:0.86:0.58:0.70), (Hypotonia:366: 4155:43:321:0.93:0.89:0.53:0.67), (General.physical.health.deterioration:171:4350:23:341:0.93:0 0.88:0.33:0.48), (Dermatitis.contact:155:4366:13:351:0.93:0.92:0.31:0.46), (Barrett's.oesophagus:4 48:4072:70:295:0.93:0.86:0.60:0.71), (Eye.pain:134:4386:10:355:0.93:0.93:0.27:0.42), (Angina.u nstable:173:4346:16:350:0.93:0.92:0.33:0.49), (Blepharitis:83:4436:2:364:0.93:0.98:0.19:0.31), (Pulmonary.congestion:285:4233:44:323:0.92:0.87:0.47:0.61), (Nephrotic.syndrome:263:4255:20: 347:0.92:0.93:0.43:0.59), (Short-bowel.syndrome:202:4316:26:341:0.92:0.89:0.37:0.52), (Dental.disorder.prophylaxis:192:4326:2 0:347:0.92:0.91:0.36:0.51), (Pancytopenia:187:4331:16:351:0.92:0.92:0.35:0.50), (Open.angle.gla ucoma:76:4442:4:363:0.92:0.95:0.17:0.29), (Respiratory.failure:268:4249:51:317:0.92:0.84:0.46: 0.59), (Sinus.tachycardia:194:4323:20:348:0.92:0.91:0.36:0.51), (Pancreatic.neuroendocrine.tumo ur:165:4352:22:346:0.92:0.88:0.32:0.47), (Dialysis:157:4360:18:350:0.92:0.90:0.31:0.46), (Phlebi tis:131:4386:11:357:0.92:0.92:0.27:0.42), (Back.disorder:408:4108:72:297:0.92:0.85:0.58:0.69), (Cytomegalovirus.viraemia:101:4415:3:366:0.92:0.97:0.22:0.35), (Discomfort:261:4254:35:335:0. 92:0.88:0.44:0.59), (Squamous.cell.carcinoma.of.lung:173:4341:21:350:0.92:0.89:0.33:0.48), (Wh ite.blood.cell.count.increased:71:4443:0:371:0.92:1.00:0.16:0.28), (Joint.swelling:429:4084:57:3 15:0.92:0.88:0.58:0.70), (Diabetic.retinal.oedema:343:4170:54:318:0.92:0.86:0.52:0.65), (Blood.c alcium.decreased:337:4176:43:329:0.92:0.89:0.51:0.64), (Pulmonary.oedema:281:4232:40:332:0. 92:0.88:0.46:0.60), (Hypoxia:227:4286:30:342:0.92:0.88:0.40:0.55), (Colon.cancer.metastatic:210: 4303:29:343:0.92:0.88:0.38:0.53), (Otitis.extema:30:4483:0:372:0.92:1.00:0.07:0.14), (Heart.rate: 329:4183:57:316:0.92:0.85:0.51:0.64), (Lipids.increased:321:4191:45:328:0.92:0.88:0.49:0.63), (Adenocarcinoma.pancreas:205:4307:25:348:0.92:0.89:0.37:0.52), (Pustular.psoriasis:162:4350:1 7:356:0.92:0.91:0.31:0.46), (Bradycardia:116:4396:9:364:0.92:0.93:0.24:0.38), (Waldenstrom's.m acroglobulinaemia:429:4082:54:320:0.92:0.89:0.57:0.70), (Meniere's.disease:216:4295:28:346:0. 92:0.89:0.38:0.54), (Lacrimation.increased:172:4339:18:356:0.92:0.91:0.33:0.48), (Pneumonia.ba cterial:58:4453:0:374:0.92:1.00:0.13:0.24), (Disseminated.intravascular.coagulation:45:4466:0:3 74:0.92:1.00:0.11:0.19), (Nuclear.magnetic.resonance.imaging:175:4335:20:355:0.92:0.90:0.33:0 0.48), (Decubitus.ulcer:138:4372:12:363:0.92:0.92:0.28:0.42), (Wound.infection:103:4407:3:372:0 0.92:0.97:0.22:0.35), (Maintenance.of.anaesthesia:132:4377:6:370:0.92:0.96:0.26:0.41), (Rash.prur itic:131:4378:14:362:0.92:0.90:0.27:0.41), (Localised.infection:286:4222:37:340:0.92:0.89:0.46: 0.60), (Cognitive.disorder:256:4252:33:344:0.92:0.89:0.43:0.58), (Chronic.graft.versus.host.disea se:107:4401:5:372:0.92:0.96:0.22:0.36), (Obsessive-compulsive.disorder:449:4058:66:312:0.92:0.87:0.59:0.70), (Vasculitis:313:4194:45:333:0.92:0.8 7:0.48:0.62), (Metabolic.disorder:125:4382:13:365:0.92:0.91:0.26:0.40), (Multiple.sclerosis.relaps e:329:4177:33:346:0.92:0.91:0.49:0.63), (Haemoptysis:36:4470:0:379:0.92:1.00:0.09:0.16), (HER 2.positive.gastric.cancer:18:4488:0:379:0.92:1.00:0.05:0.09), (Dermatomyositis:222:4283:24:356: 0.92:0.90:0.38:0.54), (Oesophageal.candidiasis:119:4386:4:376:0.92:0.97:0.24:0.39), (Extrapyra midal.disorder:113:4392:8:372:0.92:0.93:0.23:0.37), (Osteomyelitis:380:4124:52:329:0.92:0.88:0 0.54:0.67), (Convulsion:324:4180:42:339:0.92:0.89:0.49:0.63), (Nasal.dryness:228:4276:32:349:0. 92:0.88:0.40:0.54), (Factor.VIII.deficiency:114:4390:3:378:0.92:0.97:0.23:0.37), (Depressive.sym ptom:103:4401:9:372:0.92:0.92:0.22:0.35), (Postoperative.analgesia:93:4411:7:374:0.92:0.93:0.2 0:0.33), (Gingivitis:37:4467:0:381:0.92:1.00:0.09:0.16), (Mood.swings:408:4095:60:322:0.92:0.8 7:0.56:0.68), (Product.use.issue:295:4208:49:333:0.92:0.86:0.47:0.61), (Catheter.management:28 4:4219:36:346:0.92:0.89:0.45:0.60), (Dermatitis.allergic:48:4455:0:382:0.92:1.00:0.11:0.20), (Car nitine.deficiency:36:4467:0:382:0.92:1.00:0.09:0.16), (Non-small.cell.lung.cancer.recurrent:35:4468:0:382:0.92:1.00:0.08:0.15), (Vulvovaginal.dryness:396: 4106:66:317:0.92:0.86:0.56:0.67), (Chills:202:4300:26:357:0.92:0.89:0.36:0.51), (Eye.infection:1 58:4344:15:368:0.92:0.91:0.30:0.45), (Aspartate.aminotransferase.increased:27:4475:0:383:0.92: 1.00:0.07:0.12), (Polymyalgia.rheumatica:367:4134:54:330:0.92:0.87:0.53:0.66), (Weight.control: 367:4134:67:317:0.92:0.85:0.54:0.66), (Blood.glucose:314:4187:52:332:0.92:0.86:0.49:0.62), (Hi ccups:221:4280:33:351:0.92:0.87:0.39:0.54), (Haematuria:45:4456:0:384:0.92:1.00:0.10:0.19), (R aynaud's.phenomenon:372:4128:53:332:0.92:0.88:0.53:0.66), (Cholecystitis:35:4465:0:385:0.92: 1.00:0.08:0.15), (Reflux.gastritis:430:4069:59:327:0.92:0.88:0.57:0.69), (Sinus.congestion:329:41 70:55:331:0.92:0.86:0.50:0.63), (Bowel.movement.irregularity:313:4186:48:338:0.92:0.87:0.48:0 0.62), (Pancreatitis:249:4250:34:352:0.92:0.88:0.41:0.56), (Oral.contraception:245:4254:21:365:0. 92:0.92:0.40:0.56), (Pulmonary.tuberculosis:86:4413:1:385:0.92:0.99:0.18:0.31), (Cholestasis:42: 4457:0:386:0.92:1.00:0.10:0.18), (Wound:404:4093:47:341:0.92:0.90:0.54:0.68), (Colonoscopy:3 45:4152:45:343:0.92:0.88:0.50:0.64), (Respiratory.tract.congestion:306:4191:67:321:0.92:0.82:0. 49:0.61), (Invasive.ductal.breast.carcinoma:212:4285:26:362:0.92:0.89:0.37:0.52), (Drug.eruption: 54:4443:0:388:0.92:1.00:0.12:0.22), (Mouth.ulceration:308:4187:29:361:0.92:0.91:0.46:0.61), (N on-Hodgkin's.lymphoma.unspecified.histology.indolent:193:4302:23:367:0.92:0.89:0.34:0.50), (Fae ces.hard:451:4043:72:319:0.92:0.86:0.59:0.70), (Chest.discomfort:315:4179:52:339:0.92:0.86:0. 48:0.62), (Adverse.drug.reaction:272:4222:37:354:0.92:0.88:0.43:0.58), (Diabetes.mellitus.manag ement:226:4268:23:368:0.92:0.91:0.38:0.54), (Bladder.spasm:353:4140:56:336:0.92:0.86:0.51:0. 64), (Arterial.occlusive.disease:257:4236:40:352:0.92:0.87:0.42:0.57), (Prophylaxis.urinary.tract.i nfection:385:4107:57:336:0.92:0.87:0.53:0.66), (Hepatic.cancer:345:4147:48:345:0.92:0.88:0.50: 0.64), (Gastrointestinal.stromal.tumour:307:4185:36:357:0.92:0.90:0.46:0.61), (Colony.stimulatin g.factor.prophylaxis:58:4434:2:391:0.92:0.97:0.13:0.23), (Basedow's.disease:321:4170:55:339:0. 92:0.85:0.49:0.62), (Metastases.to.central.nervous.system:289:4202:47:347:0.92:0.86:0.45:0.59), (Infusion.related.reaction:237:4253:25:370:0.92:0.90:0.39:0.55), (Fluid.overload:376:4113:54:342: 0.92:0.87:0.52:0.66), (Clostridium.difficile.colitis:227:4262:29:367:0.92:0.89:0.38:0.53), (Proctal gia:117:4372:9:387:0.92:0.93:0.23:0.37), (Adenocarcinoma.gastric:62:4426:4:393:0.92:0.94:0.14: 0.24), (Paronychia:53:4434:2:396:0.92:0.96:0.12:0.21), (Generalised.oedema:40:4447:0:398:0.92: 1.00:0.09:0.17), (Blood.pressure.decreased:301:4185:46:353:0.92:0.87:0.46:0.60), (Alcoholism:2 45:4241:30:369:0.92:0.89:0.40:0.55), (Drug.abuse:209:4277:20:379:0.92:0.91:0.36:0.51), (Nephr olithiasis:410:4075:65:335:0.92:0.86:0.55:0.67), (Metastases.to.lung:441:4043:62:339:0.92:0.88: 0.57:0.69), (Chronic.sinusitis:311:4173:46:355:0.92:0.87:0.47:0.61), (Leukopenia:102:4382:2:399: 0.92:0.98:0.20:0.34), (Clostridium.difficile.infection:440:4043:64:338:0.92:0.87:0.57:0.69), (Obe sity:377:4106:53:349:0.92:0.88:0.52:0.65), (Scleroderma:318:4165:42:360:0.92:0.88:0.47:0.61), (Squamous.cell.carcinoma.of.head.and.neck:201:4282:26:376:0.92:0.89:0.35:0.50), (Status.epilept icus:174:4309:18:384:0.92:0.91:0.31:0.46), (Scan.with.contrast:97:4386:2:400:0.92:0.98:0.20:0.3 3), (Schizoaffective.disorder:460:4022:74:329:0.92:0.86:0.58:0.70), (Fungal.skin.infection:190:42 92:21:382:0.92:0.90:0.33:0.49), (Polyarthritis:313:4168:40:364:0.92:0.89:0.46:0.61), (Venous.thr ombosis:265:4216:40:364:0.92:0.87:0.42:0.57), (Heart.rate.irregular:584:3896:84:321:0.92:0.87: 0.65:0.74), (Pseudomonas.infection:375:4105:57:348:0.92:0.87:0.52:0.65), (Acute.kidney.injury:2 92:4188:38:367:0.92:0.88:0.44:0.59), (Skin.lesion:276:4204:36:369:0.92:0.88:0.43:0.58), (Heart.r ate.increased:537:3942:72:334:0.92:0.88:0.62:0.73), (Functional.gastrointestinal.disorder:274:42 05:38:368:0.92:0.88:0.43:0.57), (Hepatic.encephalopathy:261:4218:32:374:0.92:0.89:0.41:0.56), (Glioblastoma.multiforme:158:4321:16:390:0.92:0.91:0.29:0.44), (Prostatitis:274:4204:44:363:0.9 2:0.86:0.43:0.57), (Paraesthesia:482:3995:59:349:0.92:0.89:0.58:0.70), (Menopausal.symptoms:3 72:4105:50:358:0.92:0.88:0.51:0.65), (Antipyresis:64:4413:1:407:0.92:0.98:0.14:0.24), (Bronchos pasm:419:4057:80:329:0.92:0.84:0.56:0.67), (Impaired.gastric.emptying:412:4064:62:347:0.92:0. 87:0.54:0.67), (Oral.pain:329:4147:47:362:0.92:0.88:0.48:0.62), (Myasthenia.gravis:375:4100:55: 355:0.92:0.87:0.51:0.65), (Dysphagia:316:4159:49:361:0.92:0.87:0.47:0.61), (Ischaemic.stroke:28 9:4186:46:364:0.92:0.86:0.44:0.59), (Arrhythmia.prophylaxis:237:4238:33:377:0.92:0.88:0.39:0. 54), (Seborrhoeic.dermatitis:91:4384:6:404:0.92:0.94:0.18:0.31), (Behcet's.syndrome:244:4230:2 5:386:0.92:0.91:0.39:0.54), (Bowel.preparation:167:4307:16:395:0.92:0.91:0.30:0.45), (Toxicity.t o.various.agents:164:4310:11:400:0.92:0.94:0.29:0.44), (Polyneuropathy:413:4060:66:346:0.92:0 0.86:0.54:0.67), (Micturition.urgency:389:4084:60:352:0.92:0.87:0.52:0.65), (Bacteraemia:239:423 4:20:392:0.92:0.92:0.38:0.54), (Incontinence:500:3972:77:336:0.92:0.87:0.60:0.71), (Anxiolytic.t herapy:329:4143:48:365:0.92:0.87:0.47:0.61), (Schizophrenia,.paranoid.type:152:4320:14:399:0. 92:0.92:0.28:0.42), (Renal.cancer.metastatic:307:4164:52:362:0.92:0.86:0.46:0.60), (Neurogenic. bladder:302:4169:47:367:0.92:0.87:0.45:0.59), (Therapeutic.procedure:200:4271:26:388:0.92:0.8 8:0.34:0.49), (Upper.respiratory.tract.inflammation:140:4331:26:388:0.92:0.84:0.27:0.40), (Urinar y.tract.disorder:469:4001:70:345:0.92:0.87:0.58:0.69), (Growth.hormone.deficiency:415:4055:73: 342:0.92:0.85:0.55:0.67), (Therapeutic.skin.care.topical:85:4385:2:413:0.92:0.98:0.17:0.29), (An aesthesia.procedure:80:4390:2:413:0.92:0.98:0.16:0.28), (Tonsillitis:53:4417:1:414:0.92:0.98:0.1 1:0.20), (Neovascular.age-related.macular.degeneration:454:4015:77:339:0.91:0.85:0.57:0.69), (Age-related.macular.degeneration:337:4132:49:367:0.91:0.87:0.48:0.62), (Spondylitis:289:4180:43:37 3:0.91:0.87:0.44:0.58), (Tinnitus:208:4261:32:384:0.91:0.87:0.35:0.50), (Epistaxis:179:4290:23:3 93:0.91:0.89:0.31:0.46), (Irritability:175:4294:21:395:0.91:0.89:0.31:0.46), (Acromegaly:457:401 1:65:352:0.91:0.88:0.56:0.69), (Juvenile.idiopathic.arthritis:306:4162:39:378:0.91:0.89:0.45:0.59), (Platelet.count.decreased:254:4214:24:393:0.91:0.91:0.39:0.55), (Cholelithiasis:225:4243:32:38 5:0.91:0.88:0.37:0.52), (Alpha-1.anti-trypsin.deficiency:515:3952:83:335:0.91:0.86:0.61:0.71), (Drug.toxicity.prophylaxis:106:4361:4: 414:0.91:0.96:0.20:0.34), (Bladder.transitional.cell.carcinoma:52:4415:1:417:0.91:0.98:0.11:0.20), (Macular.degeneration:550:3916:89:330:0.91:0.86:0.63:0.72), (Blood.magnesium.decreased:51 3:3953:75:344:0.91:0.87:0.60:0.71), (Lupus.nephritis:510:3956:99:320:0.91:0.84:0.61:0.71), (Mo od.altered:413:4053:66:353:0.91:0.86:0.54:0.66), (Peritoneal.dialysis:347:4119:62:357:0.91:0.85: 0.49:0.62), (Glioblastoma:287:4179:37:382:0.91:0.89:0.43:0.58), (Stem.cell.transplant:286:4180: 43:376:0.91:0.87:0.43:0.58), (Foetal.exposure.during.pregnancy:363:4102:36:384:0.91:0.91:0.49: 0.63), (Inflammatory.bowel.disease:311:4153:55:366:0.91:0.85:0.46:0.60), (Ventricular.tachycard ia:252:4212:41:380:0.91:0.86:0.40:0.54), (Bronchiectasis:385:4078:66:356:0.91:0.85:0.52:0.65), (Partial.seizures:322:4141:49:373:0.91:0.87:0.46:0.60), (Drug.dependence:377:4085:48:375:0.91: 0.89:0.50:0.64), (Acidosis:229:4233:29:394:0.91:0.89:0.37:0.52), (Spinal.compression.fracture:16 2:4300:17:406:0.91:0.91:0.29:0.43), (Blood.triglycerides.increased:588:3873:78:346:0.91:0.88:0. 63:0.74), (Diverticulitis:392:4069:65:359:0.91:0.86:0.52:0.65), (Menorrhagia:337:4124:49:375:0. 91:0.87:0.47:0.61), (Musculoskeletal.chest.pain:300:4161:53:371:0.91:0.85:0.45:0.59), (Dermatiti s.acneiform:35:4426:0:424:0.91:1.00:0.08:0.14), (Calcium.deficiency:357:4103:61:364:0.91:0.85: 0.50:0.63), (Gastrointestinal.haemorrhage:325:4135:42:383:0.91:0.89:0.46:0.60), (Generalised.to nic-clonic.seizure:327:4132:44:382:0.91:0.88:0.46:0.61), (Non-small.cell.lung.cancer.metastatic:239:4220:34:392:0.91:0.88:0.38:0.53), (Influenza.like.illness:22 7:4232:34:392:0.91:0.87:0.37:0.52), (Memory.impairment:500:3958:84:343:0.91:0.86:0.59:0.70), (Basal.cell.carcinoma:423:4035:73:354:0.91:0.85:0.54:0.66), (Nerve.injury:551:3906:88:340:0.9 1:0.86:0.62:0.72), (Gastric.pH.decreased:440:4017:61:367:0.91:0.88:0.55:0.67), (Neuroendocrine. tumour:420:4037:73:355:0.91:0.85:0.54:0.66), (Haemoglobin.decreased:395:4062:58:370:0.91:0. 87:0.52:0.65), (Conjunctivitis.allergic:80:4377:3:425:0.91:0.96:0.16:0.27), (Delusion:436:4020:74: 355:0.91:0.85:0.55:0.67), (Spinal.column.stenosis:394:4062:63:366:0.91:0.86:0.52:0.65), (Transf usion:247:4209:33:396:0.91:0.88:0.38:0.54), (Preoperative.care:227:4229:22:407:0.91:0.91:0.36: 0.51), (Liver.transplant:477:3978:65:365:0.91:0.88:0.57:0.69), (Septic.shock:185:4270:14:416:0.9 1:0.93:0.31:0.46), (Hypopituitarism:432:4021:78:354:0.91:0.85:0.55:0.67), (Metastases.to.liver:40 7:4046:56:376:0.91:0.88:0.52:0.65), (Musculoskeletal.stiffness:365:4088:56:376:0.91:0.87:0.49:0 0.63), (Mania:260:4193:33:399:0.91:0.89:0.39:0.55), (Essential.thrombocythaemia:242:4211:37:39 5:0.91:0.87:0.38:0.53), (Rash.maculo-papular:37:4415:0:433:0.91:1.00:0.08:0.15), (Bronchopulmonary.aspergillosis:411:4040:73:361: 0.91:0.85:0.53:0.65), (Toothache:282:4169:37:397:0.91:0.88:0.42:0.57), (Sleep.apnoea.syndrome: 514:3936:75:360:0.91:0.87:0.59:0.70), (Cardiomyopathy:444:4006:55:380:0.91:0.89:0.54:0.67), (Post.herpetic.neuralgia:399:4051:64:371:0.91:0.86:0.52:0.65), (White.blood.cell.count.decreased: 274:4176:44:391:0.91:0.86:0.41:0.56), (Dysmenorrhoea:256:4194:34:401:0.91:0.88:0.39:0.54), (P eripheral.sensory.neuropathy:223:4227:28:407:0.91:0.89:0.35:0.51), (Brain.oedema:192:4257:22: 414:0.91:0.90:0.32:0.47), (Parenteral.nutrition:36:4413:0:436:0.91:1.00:0.08:0.14), (Electrolyte.i mbalance:304:4144:40:397:0.91:0.88:0.43:0.58), (Hot.flush:566:3881:78:360:0.91:0.88:0.61:0.72), (Supportive.care:353:4094:47:391:0.91:0.88:0.47:0.62), (Anti-infective.therapy:240:4207:35:403:0.91:0.87:0.37:0.52), (Post-traumatic.stress.disorder:555:3891:79:360:0.91:0.88:0.61:0.72), (Endometriosis:493:3953:66:373: 0.91:0.88:0.57:0.69), (Bipolar.I.disorder:439:4007:68:371:0.91:0.87:0.54:0.67), (Glucose.toleran ce.impaired:501:3944:88:352:0.91:0.85:0.59:0.69), (Trigeminal.neuralgia:455:3990:62:378:0.91: 0.88:0.55:0.67), (Hypersomnia:419:4026:74:366:0.91:0.85:0.53:0.66), (Induction.of.anaesthesia:2 90:4155:37:403:0.91:0.89:0.42:0.57), (Aplastic.anaemia:195:4250:19:421:0.91:0.91:0.32:0.47), (S inus.disorder:600:3844:85:356:0.91:0.88:0.63:0.73), (Enterocolitis:146:4298:4:437:0.91:0.97:0.2 5:0.40), (Peripheral.swelling:679:3764:79:363:0.91:0.90:0.65:0.75), (Iron.overload:333:4110:57:3 85:0.91:0.85:0.46:0.60), (Antitussive.therapy:46:4397:1:441:0.91:0.98:0.09:0.17), (Steroid.therap y:468:3974:77:366:0.91:0.86:0.56:0.68), (Hypertriglyceridaemia:395:4047:53:390:0.91:0.88:0.50: 0.64), (Helicobacter.infection:239:4203:23:420:0.91:0.91:0.36:0.52), (Prophylaxis.against.dehydr ation:151:4291:12:431:0.91:0.93:0.26:0.41), (Palmar-plantar.erythrodysaesthesia.syndrome:76:4366:6:437:0.91:0.93:0.15:0.26), (Device.related.infecti on:323:4118:52:392:0.91:0.86:0.45:0.59), (Peripheral.vascular.disorder:438:4002:59:386:0.91:0. 88:0.53:0.66), (Immune.system.disorder:468:3971:77:369:0.91:0.86:0.56:0.68), (General.anaesthe sia:273:4166:31:415:0.91:0.90:0.40:0.55), (Aspergillus.infection:383:4055:72:375:0.91:0.84:0.51: 0.63), (Dry.skin.prophylaxis:41:4397:1:446:0.91:0.98:0.08:0.16), (Pancreatic.insufficiency:321:4 116:62:386:0.91:0.84:0.45:0.59), (Panic.disorder:414:4022:64:385:0.91:0.87:0.52:0.65), (Bone.m arrow.conditioning.regimen:326:4110:52:397:0.91:0.86:0.45:0.59), (Arthropathy:613:3822:80:37 0:0.91:0.88:0.62:0.73), (Onychomycosis:385:4049:66:385:0.91:0.85:0.50:0.63), (Computerised.to mogram:212:4222:31:420:0.91:0.87:0.34:0.48), (Supraventricular.tachycardia:424:4007:67:387:0 0.91:0.86:0.52:0.65), (Ear.infection:368:4063:53:401:0.91:0.87:0.48:0.62), (Duodenal.ulcer:315:41 16:48:406:0.91:0.87:0.44:0.58), (Tinea.pedis:208:4223:28:426:0.91:0.88:0.33:0.48), (Herpes.simp lex:517:3913:86:369:0.91:0.86:0.58:0.69), (Bladder.cancer:391:4039:63:392:0.91:0.86:0.50:0.63), (Tumour.pain:212:4218:36:419:0.91:0.85:0.34:0.48), (Renal.impairment:336:4093:43:413:0.91: 0.89:0.45:0.60), (Lumbar.spinal.stenosis:285:4144:49:407:0.91:0.85:0.41:0.56), (Menopause:557: 3871:82:375:0.91:0.87:0.60:0.71), (Antiviral.treatment:505:3923:80:377:0.91:0.86:0.57:0.69), (A djuvant.therapy:473:3955:74:383:0.91:0.86:0.55:0.67), (Colorectal.cancer:380:4048:60:397:0.91: 0.86:0.49:0.62), (Chronic.hepatitis.B:292:4136:34:423:0.91:0.90:0.41:0.56), (Type.IIa.hyperlipida emia:560:3867:85:373:0.91:0.87:0.60:0.71), (Atrial.flutter:458:3969:72:386:0.91:0.86:0.54:0.67), (Nervousness:526:3900:80:379:0.91:0.87:0.58:0.70), (Spinal.osteoarthritis:411:4014:69:391:0.91: 0.86:0.51:0.64), (Plasma.cell.myeloma.recurrent:333:4092:47:413:0.91:0.88:0.45:0.59), (Embolis m:512:3912:78:383:0.91:0.87:0.57:0.69), (Depressed.mood:389:4034:61:401:0.91:0.86:0.49:0.63), (Polycythaemia.vera:593:3829:95:368:0.91:0.86:0.62:0.72), (Hypophosphataemia:314:4107:39: 425:0.91:0.89:0.42:0.58), (Pyelonephritis:233:4188:29:435:0.91:0.89:0.35:0.50), (Immunodeficie ncy:496:3922:92:375:0.90:0.84:0.57:0.68), (Vitamin.D.decreased:604:3813:90:378:0.90:0.87:0.6 2:0.72), (Stress:599:3818:77:391:0.90:0.89:0.61:0.72), (Blood.uric.acid.increased:420:3996:57:41 2:0.90:0.88:0.50:0.64), (Hypercalcaemia:379:4036:56:414:0.90:0.87:0.48:0.62), (Paroxysmal.noct umal.haemoglobinuria:489:3924:71:401:0.90:0.87:0.55:0.67), (Pneumonia.aspiration:322:4091:4 7:425:0.90:0.87:0.43:0.58), (Prophylaxis.against.graft.versus.host.disease:414:3998:57:416:0.90: 0.88:0.50:0.64), (Viral.infection:459:3952:72:402:0.90:0.86:0.53:0.66), (Small.cell.lung.cancer:36 9:4041:69:406:0.90:0.84:0.48:0.61), (Antifungal.treatment:357:4053:61:414:0.90:0.85:0.46:0.60), (Haemostasis:76:4334:3:472:0.90:0.96:0.14:0.24), (Rectal.cancer:449:3960:80:396:0.90:0.85:0.5 3:0.65), (Hyperparathyroidism.secondary:306:4102:46:431:0.90:0.87:0.42:0.56), (Palpitations:504: 3902:70:409:0.90:0.88:0.55:0.68), (Spinal.pain:456:3950:71:408:0.90:0.87:0.53:0.66), (Laxative. supportive.care:452:3954:69:410:0.90:0.87:0.52:0.65), (Transient.ischaemic.attack:586:3819:84: 396:0.90:0.87:0.60:0.71), (Mycosis.fungoides:424:3981:76:404:0.90:0.85:0.51:0.64), (Gastroenter itis:364:4041:63:417:0.90:0.85:0.47:0.60), (Blood.disorder:588:3816:84:397:0.90:0.88:0.60:0.71), (Gastrointestinal.motility.disorder:321:4083:57:424:0.90:0.85:0.43:0.57), (Blood.testosterone.de creased:643:3760:96:386:0.90:0.87:0.62:0.73), (Orthostatic.hypotension:509:3894:86:396:0.90:0. 86:0.56:0.68), (Unevaluable.event:429:3974:72:410:0.90:0.86:0.51:0.64), (Rosacea:544:3858:92: 391:0.90:0.86:0.58:0.69), (Suicide.attempt:301:4101:35:448:0.90:0.90:0.40:0.55), (Intervertebral. disc.protrusion:466:3935:61:423:0.90:0.88:0.52:0.66), (Parkinsonism:293:4108:50:434:0.90:0.85: 0.40:0.55), (Tumour.lysis.syndrome:358:4042:43:442:0.90:0.89:0.45:0.60), (Swelling:725:3674: 91:395:0.90:0.89:0.65:0.75), (Prophylaxis.against.diarrhoea:321:4078:47:439:0.90:0.87:0.42:0.57), (Hodgkin's.disease:424:3974:62:425:0.90:0.87:0.50:0.64), (Prostatic.disorder:668:3729:96:392: 0.90:0.87:0.63:0.73), (Malnutrition:228:4169:25:463:0.90:0.90:0.33:0.48), (Tuberculosis:482:391 4:60:429:0.90:0.89:0.53:0.66), (Anaemia.prophylaxis:310:4086:38:451:0.90:0.89:0.41:0.56), (Hy poaesthesia:560:3834:83:408:0.90:0.87:0.58:0.70), (Dry.mouth:504:3890:65:426:0.90:0.89:0.54: 0.67), (Arteriosclerosis:469:3925:68:423:0.90:0.87:0.53:0.66), (Hepatic.function.abnormal:378:40 16:59:432:0.90:0.86:0.47:0.61), (Hormone.therapy:550:3843:86:406:0.90:0.86:0.58:0.69), (Sjogre n's.syndrome:494:3899:80:412:0.90:0.86:0.55:0.67), (Ascites:409:3984:45:447:0.90:0.90:0.48:0. 62), (Prostatomegaly:678:3714:93:400:0.90:0.88:0.63:0.73), (Rheumatic.disorder:336:4056:52:44 1:0.90:0.87:0.43:0.58), (Herpes.virus.infection:561:3829:82:413:0.90:0.87:0.58:0.69), (Autoimmu ne.thyroiditis:557:3833:68:427:0.90:0.89:0.57:0.69), (Interstitial.lung.disease:539:3851:92:403:0. 90:0.85:0.57:0.69), (Postoperative.care:442:3947:77:419:0.90:0.85:0.51:0.64), (Dermatitis.atopic: 187:4202:24:472:0.90:0.89:0.28:0.43), (Gastrooesophageal.reflux.prophylaxis:448:3939:57:441:0 0.90:0.89:0.50:0.64), (Neutrophil.count.decreased:234:4150:37:464:0.90:0.86:0.34:0.48), (Anaphyl actic.reaction:695:3688:105:397:0.90:0.87:0.64:0.73), (Lower.respiratory.tract.infection:463:392 0:66:436:0.90:0.88:0.52:0.65), (Graft.versus.host.disease:478:3904:86:417:0.90:0.85:0.53:0.66), (Gastrointestinal.disorder.therapy:358:4024:50:453:0.90:0.88:0.44:0.59), (Hypoalbuminaemia:341: 4041:43:460:0.90:0.89:0.43:0.58), (Acute.coronary.syndrome:502:3879:87:417:0.90:0.85:0.55:0. 67), (Local.anaesthesia:339:4042:47:457:0.90:0.88:0.43:0.57), (Hyperphosphataemia:466:3914:77: 428:0.90:0.86:0.52:0.65), (Acute.myocardial.infarction:430:3950:64:441:0.90:0.87:0.49:0.63), (C ystic.fibrosis:563:3815:100:407:0.90:0.85:0.58:0.69), (Panic.attack:659:3718:94:414:0.90:0.88:0. 61:0.72), (Adrenal.insufficiency:485:3892:71:437:0.90:0.87:0.53:0.66), (Bladder.disorder:693:36 83:94:415:0.90:0.88:0.63:0.73), (Urinary.retention:525:3849:77:434:0.90:0.87:0.55:0.67), (Pancre atic.carcinoma.metastatic:370:4004:57:454:0.90:0.87:0.45:0.59), (Respiratory.disorder:613:3759: 89:424:0.89:0.87:0.59:0.71), (Restlessness:499:3873:79:434:0.89:0.86:0.53:0.66), (Cytomegalovir us.infection:473:3899:64:449:0.89:0.88:0.51:0.65), (Colitis:626:3745:93:421:0.89:0.87:0.60:0.71), (Osteoporosis.postmenopausal:621:3750:91:423:0.89:0.87:0.59:0.71), (Medication.dilution:326: 4045:50:464:0.89:0.87:0.41:0.56), (Alopecia:664:3706:81:434:0.89:0.89:0.60:0.72), (Mucosal.infl ammation:511:3858:86:430:0.89:0.86:0.54:0.66), (Respiratory.tract.infection:531:3836:73:445:0. 89:0.88:0.54:0.67), (Hallucination:564:3802:95:424:0.89:0.86:0.57:0.68), (Eye.disorder:669:3696:99:421:0.89:0.87:0.61:0.72), (Emphysema:685:3678:72:450:0.89:0.90:0.60:0.72), (Oral.herpes:5 41:3822:81:441:0.89:0.87:0.55:0.67), (Pleural.effusion:434:3929:71:451:0.89:0.86:0.49:0.62), (M ental.disorder:625:3737:97:426:0.89:0.87:0.59:0.71), (Thyroid.cancer:517:3845:90:433:0.89:0.85: 0.54:0.66), (Skin.infection:482:3880:79:444:0.89:0.86:0.52:0.65), (Haemorrhage.prophylaxis:396: 3966:66:457:0.89:0.86:0.46:0.60), (Skin.disorder:582:3779:87:437:0.89:0.87:0.57:0.69), (Thyroi dectomy:511:3849:90:435:0.89:0.85:0.54:0.66), (Peptic.ulcer:493:3867:72:453:0.89:0.87:0.52:0. 65), (Haemorrhage:460:3900:72:453:0.89:0.86:0.50:0.64), (Rhinorrhoea:473:3886:78:448:0.89:0. 86:0.51:0.64), (Delirium:397:3962:62:464:0.89:0.86:0.46:0.60), (Hiatus.hernia:670:3688:99:428:0 0.89:0.87:0.61:0.72), (Hepatitis.B:558:3800:78:449:0.89:0.88:0.55:0.68), (Hypogammaglobulinae mia:696:3660:115:414:0.89:0.86:0.63:0.72), (Cataract:440:3915:85:445:0.89:0.84:0.50:0.62), (Ne oplasm:544:3810:93:438:0.89:0.85:0.55:0.67), (Cerebral.infarction:566:3787:110:422:0.89:0.84: 0.57:0.68), (Blood.glucose.abnormal:587:3764:90:444:0.89:0.87:0.57:0.69), (Antiallergic.therapy: 486:3865:79:455:0.89:0.86:0.52:0.65), (Malaise:536:3814:88:447:0.89:0.86:0.55:0.67), (Gastric.c ancer:454:3895:69:467:0.89:0.87:0.49:0.63), (Oesophagitis:496:3852:82:455:0.89:0.86:0.52:0.65), (Peripheral.arterial.occlusive.disease:599:3748:104:434:0.89:0.85:0.58:0.69), (Pneumocystis.jir ovecii.pneumonia:493:3854:87:451:0.89:0.85:0.52:0.65), (Folate.deficiency:429:3918:67:471:0.8 9:0.86:0.48:0.61), (Ill-defined.disorder:716:3629:96:444:0.89:0.88:0.62:0.73), (Haemolytic.uraemic.syndrome:538:380 7:91:449:0.89:0.86:0.55:0.67), (Thrombocytopenia:612:3732:106:435:0.89:0.85:0.58:0.69), (Mant le.cell.lymphoma:602:3742:101:440:0.89:0.86:0.58:0.69), (Somnolence:778:3565:104:438:0.89:0 0.88:0.64:0.74), (Ischaemic.heart.disease.prophylaxis:536:3807:92:450:0.89:0.85:0.54:0.66), (Non-small.cell.lung.cancer.stage.IV:384:3958:74:469:0.89:0.84:0.45:0.59), (Sleep.disorder.therapy:67 3:3668:95:449:0.89:0.88:0.60:0.71), (Bronchitis.chronic:621:3720:93:451:0.89:0.87:0.58:0.70), (Hyperchlorhydria:556:3785:84:460:0.89:0.87:0.55:0.67), (Renal.disorder:703:3637:98:447:0.89: 0.88:0.61:0.72), (Erythema:357:3980:50:498:0.89:0.88:0.42:0.57), (Diagnostic.procedure:463:387 1:74:477:0.89:0.86:0.49:0.63), (Vitamin.B12.deficiency:715:3618:109:443:0.89:0.87:0.62:0.72), (Nervous.system.disorder:598:3735:88:464:0.89:0.87:0.56:0.68), (Immunisation:569:3761:76:479: 0.89:0.88:0.54:0.67), (Skin.ulcer:439:3891:75:480:0.89:0.85:0.48:0.61), (Conjunctivitis:326:4004: 47:508:0.89:0.87:0.39:0.54), (Vertigo:644:3685:93:463:0.89:0.87:0.58:0.70), (Oral.candidiasis:6 19:3710:96:460:0.89:0.87:0.57:0.69), (Colorectal.cancer.metastatic:449:3880:73:483:0.89:0.86:0. 48:0.62), (Tremor:757:3571:108:449:0.89:0.88:0.63:0.73), (Cardiac.failure.chronic:671:3656:125: 433:0.89:0.84:0.61:0.71), (Blood.pressure.management:647:3679:106:453:0.89:0.86:0.59:0.70), (Major.depression:763:3562:95:465:0.89:0.89:0.62:0.73), (Urinary.incontinence:739:3586:108:45 2:0.89:0.87:0.62:0.73), (Antiplatelet.therapy:683:3642:120:440:0.89:0.85:0.61:0.71), (Embolism. venous:652:3672:94:467:0.89:0.87:0.58:0.70), (Viral.upper.respiratory.tract.infection:342:3982:5 9:502:0.89:0.85:0.41:0.55), (Renal.failure:639:3684:111:451:0.88:0.85:0.59:0.69), (Flatulence:62 1:3702:100:462:0.88:0.86:0.57:0.69), (Allergy.prophylaxis:407:3916:59:503:0.88:0.87:0.45:0.59), (Blood.glucose.increased:668:3654:89:474:0.88:0.88:0.58:0.70), (Stent.placement:661:3661:107: 456:0.88:0.86:0.59:0.70), (Breakthrough.pain:630:3692:100:463:0.88:0.86:0.58:0.69), (Affective. disorder:814:3507:110:454:0.88:0.88:0.64:0.74), (Weight.decreased:696:3625:99:465:0.88:0.88: 0.60:0.71), (Osteopenia:833:3487:119:446:0.88:0.88:0.65:0.75), (Lung.adenocarcinoma:578:3742: 105:460:0.88:0.85:0.56:0.67), (Hyponatraemia:475:3845:81:484:0.88:0.85:0.50:0.63), (Hepatic.ci rrhosis:545:3774:88:478:0.88:0.86:0.53:0.66), (Convulsion.prophylaxis:507:3812:89:477:0.88:0. 85:0.52:0.64), (Pain.prophylaxis:670:3648:122:445:0.88:0.85:0.60:0.70), (Asthenia:609:3705:91: 480:0.88:0.87:0.56:0.68), (Sciatica:562:3752:83:488:0.88:0.87:0.54:0.66), (Pharyngitis:439:3875: 89:482:0.88:0.83:0.48:0.61), (Erectile.dysfunction:845:3468:107:465:0.88:0.89:0.65:0.75), (Hypo magnesaemia:707:3605:124:449:0.88:0.85:0.61:0.71), (Chronic.gastritis:778:3533:176:398:0.88: 0.82:0.66:0.73), (Surgery:608:3703:103:471:0.88:0.86:0.56:0.68), (Hormone-refractory.prostate.cancer:532:3779:100:474:0.88:0.84:0.53:0.65), (Hypoglycaemia:469:3840:94: 482:0.88:0.83:0.49:0.62), (Immunodeficiency.common.variable:887:3419:129:450:0.88:0.87:0.6 6:0.75), (Nephrogenic.anaemia:675:3630:143:437:0.88:0.83:0.61:0.70), (Idiopathic.pulmonary.fib rosis:817:3487:106:475:0.88:0.89:0.63:0.74), (Iron.deficiency:730:3573:108:474:0.88:0.87:0.61: 0.71), (Staphylococcal.infection:658:3645:103:479:0.88:0.86:0.58:0.69), (Renal.cancer:735:3567: 126:457:0.88:0.85:0.62:0.72), (Prophylaxis.against.transplant.rejection:765:3536:128:456:0.88:0. 86:0.63:0.72), (Smoking.cessation.therapy:746:3555:103:481:0.88:0.88:0.61:0.72), (Sedative.ther apy:638:3663:94:490:0.88:0.87:0.57:0.69), (Antacid.therapy:615:3685:97:488:0.88:0.86:0.56:0.6 8), (Antiemetic.supportive.care:542:3758:101:484:0.88:0.84:0.53:0.65), (Pollakiuria:642:3657:10 2:484:0.88:0.86:0.57:0.69), (Lymphoma:765:3531:113:476:0.88:0.87:0.62:0.72), (Lung.infection: 560:3735:92:498:0.88:0.86:0.53:0.65), (Blood.pressure:943:3351:147:444:0.88:0.87:0.68:0.76), (Breast.cancer.female:705:3588:114:478:0.88:0.86:0.60:0.70), (Bacterial.infection:636:3656:99:4 94:0.88:0.87:0.56:0.68), (Immunosuppressant.drug.therapy:803:3487:129:466:0.88:0.86:0.63:0.7 3), (Coagulopathy:697:3592:123:473:0.88:0.85:0.60:0.70), (Anxiety.disorder:669:3620:100:496:0 0.88:0.87:0.57:0.69), (Hyperthyroidism:789:3499:110:487:0.88:0.88:0.62:0.73), (Bone.pain:681:36 06:110:488:0.88:0.86:0.58:0.69), (Bone.disorder:981:3305:140:459:0.88:0.88:0.68:0.77), (Cancer. pain:900:3385:169:431:0.88:0.84:0.68:0.75), (Prostate.cancer.metastatic:616:3669:118:482:0.88: 0.84:0.56:0.67), (Lung.disorder:883:3401:124:477:0.88:0.88:0.65:0.75), (Drug.therapy:699:3585: 111:490:0.88:0.86:0.59:0.70), (Hepatocellular.carcinoma:553:3731:99:502:0.88:0.85:0.52:0.65), (Blood.potassium.decreased:784:3499:119:483:0.88:0.87:0.62:0.72), (Immune.thrombocytopenic. purpura:689:3593:134:469:0.88:0.84:0.59:0.70), (Dermatitis:467:3815:81:522:0.88:0.85:0.47:0.6 1), (Multiple.allergies:886:3395:106:498:0.88:0.89:0.64:0.75), (Essential.hypertension:737:3542: 107:499:0.88:0.87:0.60:0.71), (Antidepressant.therapy:827:3450:117:491:0.88:0.88:0.63:0.73), (E lectrolyte.substitution.therapy:684:3592:111:498:0.88:0.86:0.58:0.69), (Hormone.replacement.the rapy:933:3342:126:484:0.88:0.88:0.66:0.75), (Diabetic.neuropathy:859:3412:126:488:0.87:0.87: 0.64:0.74), (Dementia:733:3537:118:497:0.87:0.86:0.60:0.70), (Iron.deficiency.anaemia:919:3350: 178:438:0.87:0.84:0.68:0.75), (Muscle.spasticity:724:3544:112:505:0.87:0.87:0.59:0.70), (Dysuri a:643:3623:117:502:0.87:0.85:0.56:0.68), (Wheezing:1067:3198:123:497:0.87:0.90:0.68:0.77), (Nasal.congestion:771:3494:109:511:0.87:0.88:0.60:0.71), (Myocardial.ischaemia:718:3547:119:5 01:0.87:0.86:0.59:0.70), (Hypovitaminosis:712:3553:112:508:0.87:0.86:0.58:0.70), (Acute.lymph ocytic.leukaemia:665:3600:113:507:0.87:0.85:0.57:0.68), (Rhinitis:624:3641:105:515:0.87:0.86: 0.55:0.67), (Agitation:798:3466:138:483:0.87:0.85:0.62:0.72), (Liver.disorder:677:3587:113:508: 0.87:0.86:0.57:0.69), (Colon.cancer:748:3515:120:502:0.87:0.86:0.60:0.71), (Cardiovascular.diso rder:867:3395:110:513:0.87:0.89:0.63:0.74), (Acne:734:3528:113:510:0.87:0.87:0.59:0.70), (Urti caria:697:3565:99:524:0.87:0.88:0.57:0.69), (Pain.management:821:3436:137:491:0.87:0.86:0.63: 0.72), (Muscle.relaxant.therapy:931:3325:132:497:0.87:0.88:0.65:0.75), (Abdominal.distension:5 34:3721:87:543:0.87:0.86:0.50:0.63), (Polyuria:829:3425:137:494:0.87:0.86:0.63:0.72), (Procedu ral.pain:636:3617:104:528:0.87:0.86:0.55:0.67), (Fatigue:830:3422:129:504:0.87:0.87:0.62:0.72), (Renal.transplant:919:3332:129:505:0.87:0.88:0.65:0.74), (Ulcer:831:3420:129:505:0.87:0.87:0.6 2:0.72), (Metastatic.renal.cell.carcinoma:730:3521:147:487:0.87:0.83:0.60:0.70), (Antiinflammat ory.therapy:701:3549:115:520:0.87:0.86:0.57:0.69), (Dehydration:615:3635:122:513:0.87:0.83:0. 55:0.66), (Candida.infection:701:3548:119:517:0.87:0.85:0.58:0.69), (HER-2.positive.breast.cancer:439:3810:72:564:0.87:0.86:0.44:0.58), (Ankylosing.spondylitis:948:3299: 127:511:0.87:0.88:0.65:0.75), (Relapsing-remitting.multiple.sclerosis:694:3552:110:529:0.87:0.86:0.57:0.68), (Cataplexy:1242:3003:207:4 33:0.87:0.86:0.74:0.80), (Psychotic.disorder:826:3417:143:499:0.87:0.85:0.62:0.72), (Pancreatic.c arcinoma:601:3642:113:529:0.87:0.84:0.53:0.65), (Hyperglycaemia:805:3434:151:495:0.87:0.84: 0.62:0.71), (Anaesthesia:634:3605:100:546:0.87:0.86:0.54:0.66), (Blood.cholesterol.abnormal:11 39:3099:151:496:0.87:0.88:0.70:0.78), (Dementia.Alzheimer's.type:769:3468:147:501:0.87:0.84: 0.61:0.70), (B-cell.lymphoma:750:3487:119:529:0.87:0.86:0.59:0.70), (Cystitis:707:3528:116:534:0.87:0.86:0.5 7:0.69), (Cerebrovascular.accident:951:3282:140:512:0.87:0.87:0.65:0.74), (Systemic.lupus.eryth ematosus:969:3263:142:511:0.87:0.87:0.65:0.75), (Stomatitis:753:3479:143:510:0.87:0.84:0.60:0 0.70), (Gait.disturbance:874:3356:134:521:0.87:0.87:0.63:0.73), (Diffuse.large.B-cell.lymphoma:778:3452:130:525:0.87:0.86:0.60:0.70), (Metastatic.malignant.melanoma:779:34 50:142:514:0.87:0.85:0.60:0.70), (Febrile.neutropenia:683:3546:127:529:0.87:0.84:0.56:0.68), (N eoplasm.malignant:1035:3193:129:528:0.87:0.89:0.66:0.76), (Cellulitis:710:3518:118:539:0.87:0 0.86:0.57:0.68), (Neck.pain:770:3456:114:545:0.87:0.87:0.59:0.70), (Myelofibrosis:903:3322:120: 540:0.86:0.88:0.63:0.73), (Attention.deficit/hyperactivity.disorder:923:3301:148:513:0.86:0.86:0. 64:0.74), (Type.1.diabetes.mellitus:859:3365:138:523:0.86:0.86:0.62:0.72), (Non-Hodgkin's.lymphoma:749:3474:134:528:0.86:0.85:0.59:0.69), (Cardiac.failure.congestive:965:32 57:150:513:0.86:0.87:0.65:0.74), (Blood.pressure.increased:1058:3163:154:510:0.86:0.87:0.67:0. 76), (Chronic.kidney.disease:854:3366:146:519:0.86:0.85:0.62:0.72), (Pulmonary.hypertension:11 20:3098:138:529:0.86:0.89:0.68:0.77), (Adverse.event:692:3523:132:538:0.86:0.84:0.56:0.67), (B lood.cholesterol:1328:2886:152:519:0.86:0.90:0.72:0.80), (Irritable.bowel.syndrome:859:3353:1 19:554:0.86:0.88:0.61:0.72), (Haemorrhoids:679:3533:116:557:0.86:0.85:0.55:0.67), (Musculosk eletal.pain:891:3320:141:533:0.86:0.86:0.63:0.73), (Sepsis:757:3454:149:525:0.86:0.84:0.59:0.6 9), (Restless.legs.syndrome:1097:3112:143:533:0.86:0.88:0.67:0.76), (Renal.cell.carcinoma:741:3 468:136:540:0.86:0.84:0.58:0.69), (Hypocalcaemia:781:3427:161:516:0.86:0.83:0.60:0.70), (Hyp ertonic.bladder:1012:3195:151:527:0.86:0.87:0.66:0.75), (Neutropenia:907:3299:136:543:0.86:0. 87:0.63:0.73), (Osteoporosis.prophylaxis:837:3369:143:536:0.86:0.85:0.61:0.71), (Fluid.replacem ent:692:3513:129:551:0.86:0.84:0.56:0.67), (Immunosuppression:922:3282:161:520:0.86:0.85:0. 64:0.73), (Blood.pressure.measurement:1317:2886:151:531:0.86:0.90:0.71:0.79), (Chemotherapy: 999:3202:127:557:0.86:0.89:0.64:0.74), (Tachycardia:886:3315:144:540:0.86:0.86:0.62:0.72), (Pr oductive.cough:696:3505:137:547:0.86:0.84:0.56:0.67), (Hyperuricaemia:1389:2810:222:464:0.8 6:0.86:0.75:0.80), (Dry.skin:692:3506:119:568:0.86:0.85:0.55:0.67), (Fibromyalgia:1321:2876:17 1:517:0.86:0.89:0.72:0.79), (Routine.health.maintenance:892:3305:157:531:0.86:0.85:0.63:0.72), (Eczema:866:3330:178:511:0.86:0.83:0.63:0.72), (Blood.pressure.abnormal:1354:2841:165:525: 0.86:0.89:0.72:0.80), (Metastases.to.bone:967:3227:171:520:0.86:0.85:0.65:0.74), (Chest.pain:11 51:3041:151:542:0.86:0.88:0.68:0.77), (Rhinitis.allergic:1153:3038:189:505:0.86:0.86:0.70:0.77), (Myocardial.infarction:1021:3170:180:514:0.86:0.85:0.67:0.75), (Ovarian.cancer:647:3543:118: 577:0.86:0.85:0.53:0.65), (Bipolar.disorder:1248:2937:176:524:0.86:0.88:0.70:0.78), (Oropharyn geal.pain:573:3612:100:600:0.86:0.85:0.49:0.62), (Thyroid.disorder:1414:2766:185:520:0.86:0.8 8:0.73:0.80), (Gastric.ulcer:1224:2956:228:477:0.86:0.84:0.72:0.78), (Influenza:765:3415:113:59 2:0.86:0.87:0.56:0.68), (Dyslipidaemia:1483:2695:212:495:0.86:0.87:0.75:0.81), (Antifungal.prop hylaxis:1088:3084:189:524:0.85:0.85:0.67:0.75), (Breast.cancer.metastatic:877:3295:139:574:0.8 5:0.86:0.60:0.71), (Gastritis.prophylaxis:1197:2970:221:497:0.85:0.84:0.71:0.77), (Arrhythmia:12 56:2909:188:532:0.85:0.87:0.70:0.78), (Dizziness:964:3201:162:558:0.85:0.86:0.63:0.73), (Fluid. retention:1177:2987:149:572:0.85:0.89:0.67:0.77), (Herpes.zoster:1113:3047:203:522:0.85:0.85: 0.68:0.75), (Myalgia:989:3171:151:574:0.85:0.87:0.63:0.73), (Sinusitis:1038:3121:128:598:0.85: 0.89:0.63:0.74), (Hypertension:4046:111:717:11:0.85:0.85:1.00:0.92), (Myelodysplastic.syndrom e:1212:2945:215:513:0.85:0.85:0.70:0.77), (Abdominal.discomfort:1110:3047:158:570:0.85:0.88:0.66:0.75), (Thrombosis:1189:2966:161:569:0.85:0.88:0.68:0.77), (Acute.myeloid.leukaemia:883:3271:159:572:0.85:0.85:0.61:0.71), (Oedema.peripheral:987:3165:155:578:0.85:0.86:0.63:0.73), (Antibiotic.therapy:974:3178:154:579:0.85:0.86:0.63:0.73), (Hypotension:926:3226:162:571:0.8 5:0.85:0.62:0.72), (Inflammation:1151:2999:158:577:0.85:0.88:0.67:0.76), (Upper.respiratory.trac t.infection:803:3347:112:623:0.85:0.88:0.56:0.69), (Hyperkalaemia:1023:3124:169:569:0.85:0.8 6:0.64:0.73), (Constipation.prophylaxis:988:3159:170:568:0.85:0.85:0.63:0.73), (Gout:1382:2762: 167:574:0.85:0.89:0.71:0.79), (Glaucoma:1269:2875:181:560:0.85:0.88:0.69:0.77), (Gastritis:15 05:2637:234:509:0.85:0.87:0.75:0.80), (Fungal.infection:1028:3113:162:582:0.85:0.86:0.64:0.73), (Angina.pectoris:1228:2912:207:538:0.85:0.86:0.70:0.77), (Decreased.appetite:1021:3119:196: 549:0.85:0.84:0.65:0.73), (Schizophrenia:1167:2972:199:547:0.85:0.85:0.68:0.76), (Arthritis:151 4:2624:186:561:0.85:0.89:0.73:0.80), (Gastric.disorder:1219:2917:167:582:0.85:0.88:0.68:0.76), (Narcolepsy:1577:2558:295:455:0.85:0.84:0.78:0.81), (Blood.cholesterol.increased:1777:2356:25 6:496:0.85:0.87:0.78:0.83), (Vitamin.D.deficiency:1260:2873:187:565:0.85:0.87:0.69:0.77), (Chr onic.hepatitis.C:1174:2958:217:536:0.85:0.84:0.69:0.76), (Sedation:1034:3097:184:570:0.85:0.8 5:0.64:0.73), (Psoriatic.arthropathy:1421:2706:187:571:0.84:0.88:0.71:0.79), (Bronchitis:1262:28 63:178:582:0.84:0.88:0.68:0.77), (Prophylaxis.against.gastrointestinal.ulcer:1527:2597:275:486:0 0.84:0.85:0.76:0.80), (Malignant.melanoma:963:3161:167:594:0.84:0.85:0.62:0.72), (Coronary.arte ry.disease:1162:2960:166:597:0.84:0.88:0.66:0.75), (Pulmonary.embolism:1278:2842:176:589:0. 84:0.88:0.68:0.77), (Colitis.ulcerative:1093:3027:149:616:0.84:0.88:0.64:0.74), (Seasonal.allergy: 1201:2916:177:591:0.84:0.87:0.67:0.76), (Migraine:1523:2592:198:572:0.84:0.88:0.73:0.80), (Dr y.eye:1210:2904:173:598:0.84:0.87:0.67:0.76), (Lung.neoplasm.malignant:1084:3030:148:623:0. 84:0.88:0.64:0.74), (Cardiovascular.event.prophylaxis:1185:2925:177:598:0.84:0.87:0.66:0.75), (Contraception:1180:2930:189:586:0.84:0.86:0.67:0.75), (Osteoarthritis:1304:2803:178:600:0.84: 0.88:0.68:0.77), (Diuretic.therapy:1258:2846:170:611:0.84:0.88:0.67:0.76), (Abdominal.pain.upp er:1081:3023:185:596:0.84:0.85:0.64:0.73), (HIV.infection:1131:2971:166:617:0.84:0.87:0.65:0. 74), (Cardiac.failure:1427:2673:234:551:0.84:0.86:0.72:0.78), (Cardiac.disorder:1608:2489:214:5 74:0.84:0.88:0.74:0.80), (Oedema:1432:2663:211:579:0.84:0.87:0.71:0.78), (Pulmonary.arterial.h ypertension:1382:2713:192:598:0.84:0.88:0.70:0.78), (Parkinson's.disease:1313:2780:214:578:0. 84:0.86:0.69:0.77), (Chronic.lymphocytic.leukaemia:1241:2849:174:621:0.84:0.88:0.67:0.76), (G astrointestinal.disorder:1153:2936:178:618:0.84:0.87:0.65:0.74), (Antiviral.prophylaxis:1210:287 7:196:602:0.84:0.86:0.67:0.75), (Neuropathy.peripheral:1706:2379:241:559:0.84:0.88:0.75:0.81), (Pain.in.extremity:1196:2889:175:625:0.84:0.87:0.66:0.75), (Deep.vein.thrombosis:1400:2682:19 9:604:0.84:0.88:0.70:0.78), (Neuralgia:1587:2494:213:591:0.84:0.88:0.73:0.80), (Chronic.myeloi d.leukaemia:1183:2895:206:601:0.83:0.85:0.66:0.75), (Benign.prostatic.hyperplasia:1488:2589:2 30:578:0.83:0.87:0.72:0.79), (Mineral.supplementation:1406:2671:189:619:0.83:0.88:0.69:0.78), (Dyspnoea:1448:2628:178:631:0.83:0.89:0.70:0.78), (Nasopharyngitis:1060:3013:195:617:0.83:0. 84:0.63:0.72), (Seizure:1498:2574:210:603:0.83:0.88:0.71:0.79), (Offlabel.use:1290:2778:204:61 3:0.83:0.86:0.68:0.76), (Antibiotic.prophylaxis:1135:2932:198:620:0.83:0.85:0.65:0.74), (Epileps y:1577:2487:259:562:0.83:0.86:0.74:0.79), (Muscle.spasms:1580:2475:211:619:0.83:0.88:0.72:0. 79), (Cerebrovascular.accident.prophylaxis:1576:2477:235:597:0.83:0.87:0.73:0.79), (Hypokalae mia:1452:2599:217:617:0.83:0.87:0.70:0.78), (Depression:3046:1001:504:334:0.83:0.86:0.90:0.8 8), (Pruritus:1603:2444:236:602:0.83:0.87:0.73:0.79), (Hypercholesterolaemia:1783:2261:210:63 1:0.83:0.89:0.74:0.81), (Crohn's.disease:1515:2524:211:635:0.83:0.88:0.70:0.78), (Rash:1355:268 4:194:652:0.83:0.87:0.68:0.76), (Prostate.cancer:1397:2640:204:644:0.83:0.87:0.68:0.77), (Non-small.cell.lung.cancer:1294:2743:226:622:0.83:0.85:0.68:0.75), (Osteoporosis:2184:1850:345:50 6:0.83:0.86:0.81:0.84), (Infection.prophylaxis:1706:2326:259:594:0.83:0.87:0.74:0.80), (Hyperlip idaemia:1889:2142:259:595:0.83:0.88:0.76:0.82), (Arthralgia:1712:2316:243:614:0.82:0.88:0.74: 0.80), (Rheumatoid.arthritis:2615:1410:456:404:0.82:0.85:0.87:0.86), (Type.2.diabetes.mellitus:2 361:1664:332:528:0.82:0.88:0.82:0.85), (Hepatitis.C:1374:2651:201:659:0.82:0.87:0.68:0.76), (P neumonia:1531:2491:243:620:0.82:0.86:0.71:0.78), (Psoriasis:1756:2265:282:582:0.82:0.86:0.75: 0.80), (Prophylaxis.of.nausea.and.vomiting:1284:2736:233:632:0.82:0.85:0.67:0.75), (Anticoagul ant.therapy:1676:2342:216:651:0.82:0.89:0.72:0.79), (Abdominal.pain:1144:2874:191:676:0.82: 0.86:0.63:0.73), (Diabetes.mellitus:3016:1000:526:343:0.82:0.85:0.90:0.87), (Chronic.obstructive.pulmonary.disease:1819:2195:231:640:0.82:0.89:0.74:0.81), (Sleep.disorder:1952:2058:288:587: 0.82:0.87:0.77:0.82), (Atrial.fibrillation:2376:1632:360:517:0.82:0.87:0.82:0.84), (Infection:1587: 2419:221:658:0.82:0.88:0.71:0.78), (Insomnia:2826:1179:446:434:0.82:0.86:0.87:0.87), (Dyspep sia:1665:2340:201:679:0.82:0.89:0.71:0.79), (Constipation:2723:1281:403:478:0.82:0.87:0.85:0. 86), (Gastrooesophageal.reflux.disease:2732:1271:435:447:0.82:0.86:0.86:0.86), (Hypothyroidis m:2376:1622:319:568:0.82:0.88:0.81:0.84), (Hypersensitivity:1960:2038:296:591:0.82:0.87:0.77: 0.82), (Vitamin.supplementation:1724:2272:255:634:0.82:0.87:0.73:0.80), (Urinary.tract.infectio n:1602:2391:215:677:0.82:0.88:0.70:0.78), (Breast.cancer:1390:2600:201:694:0.82:0.87:0.67:0.7 6), (Thrombosis.prophylaxis:2018:1969:293:605:0.82:0.87:0.77:0.82), (Multiple.sclerosis:1913:2 073:344:555:0.82:0.85:0.78:0.81), (Anxiety:2614:1369:397:505:0.82:0.87:0.84:0.85), (Supplemen tation.therapy:1829:2152:285:619:0.81:0.87:0.75:0.80), (Pain:3756:223:870:36:0.81:0.81:0.99:0. 89), (Analgesic.therapy:1442:2537:234:672:0.81:0.86:0.68:0.76), (Anaemia:1836:2138:262:649:0 0.81:0.88:0.74:0.80), (Premedication:1698:2271:249:667:0.81:0.87:0.72:0.79), (Asthma:2335:1633: 372:545:0.81:0.86:0.81:0.84), (Nutritional.supplementation:1449:2513:214:709:0.81:0.87:0.67:0 0.76), (Headache:1855:2105:286:639:0.81:0.87:0.74:0.80), (Back.pain:2117:1840:346:582:0.81:0.8 6:0.78:0.82), (Diarrhoea:1997:1960:312:616:0.81:0.86:0.76:0.81), (Prophylaxis:3749:204:889:43: 0.81:0.81:0.99:0.89), (Pyrexia:1795:2138:290:662:0.81:0.86:0.73:0.79), (Cough:1637:2295:250:7 03:0.80:0.87:0.70:0.77), (Nausea:2555:1357:464:509:0.80:0.85:0.83:0.84), (Vomiting:1612:2289: 230:754:0.80:0.88:0.68:0.77), (Plasma.cell.myeloma:2256:1627:432:570:0.79:0.84:0.80:0.82)

### REFERENCE SIGNS LIST

10:training device
20:prediction device
101:processing part
201:processing part

1. A method for predicting a new indication for a known drug of interest or its equivalent substance, comprising:
a step of predicting a new indication for the known drug of interest or its equivalent substance using an artificial intelligence model trained based on test data which is information about adverse events and/or side effects reported for the known drug of interest or its equivalent substance.

2. The prediction method according to claim 1, wherein the information about adverse events and/or side effects corresponds to the presence or absence of multiple adverse events and/or side effects, or occurrence frequencies thereof.

3. The prediction method according to claim 1, wherein the artificial intelligence model corresponds to one indication.

4. The prediction method according to claim 1, wherein the artificial intelligence model corresponds to multiple indications.

5. A device for predicting a new indication for a known drug of interest or its equivalent substance,
comprising a processing part,
wherein the processing part is configured to predict a new indication for the known drug of interest or its equivalent substance using an artificial intelligence model trained based on test data which is information about adverse events and/or side effects reported for the known drug of interest or its equivalent substance.

6. A computer program for predicting a new indication for a known drug of interest or its equivalent substance,
executable by a computer to cause the computer to execute processing including a step of predicting a new indication for the known drug of interest or its equivalent substance using an artificial intelligence model trained based on test data which is information about adverse events and/or side effects reported for the known drug of interest or its equivalent substance.

7. A method for training an artificial intelligence model,
comprising training an artificial intelligence model by means of a set of training data,
wherein each item of training data is data in which (I) information about adverse events and/or side effects reported for individual known drugs is/are associated with (II) indication data reported for the known drugs, and
wherein the artificial intelligence model predicts a new indication for a known drug of interest or its equivalent substance.

8. The training method according to claim 7,
wherein each item of the training data is generated by linking a label indicating an indication for the known drug and information about adverse events and/or side effects reported for the known drug by means of a label indicating the name of the known drug.

9. The training method according to claim 7,
wherein the information about adverse event and/or side effects corresponds to the presence or absence of multiple adverse events and/or side effects; or occurrence frequencies of adverse events and/or side effects.

10. The training method according to claim 7,
wherein the artificial intelligence model corresponds to one indication.

11. The training method according to claim 7,
wherein the artificial intelligence model corresponds to multiple indications.

12. A device for training an artificial intelligence model,
comprising a processing part,
wherein the processing part is configured to train an artificial intelligence model by means of a set of training data,
wherein each item of training data is data in which (I) information about an adverse event and/or side effect reported for an individual known drug is associated with (II) indication data reported for the known drug, and
wherein the artificial intelligence model predicts a new indication for a known drug of interest or its equivalent substance.

13. A program for training an artificial intelligence model, executable by a computer to cause the computer to execute processing including a step of training an artificial intelligence model by means of a set of training data,
wherein each item of training data is data in which information about an adverse event and/or side effect reported for an individual known drug is associated with indication data reported for the known drug, and
wherein the artificial intelligence model predicts a new indication for a known drug of interest or its equivalent substance.

14. A composition containing a drug selected from a drug list shown in the description in order to use the drug in treatment or prevention of a new indication predicted for the drug by the prediction method according to claim 1.
